(19) United States
(12) Patent Application Publication
(10) Pub. No.: US 2014/0259212 A1
(43) Pub. Date: Sep. 11, 2014

(54) Process for the Production of Fine Chemicals

(71) Applicants: Gunnar Plesch, Potsdam (DE); Astrid Blau, Stahnsdorf (DE); Michael Manfred Herold, Berlin (DE); Beate Kamlage, Berlin (DE); Birgit Wendel, Berlin (DE); Piotr Puzio, Mariakerke (Gent) (BE); Stefan Henkes, Potsdam (DE); Volker Haake, Berlin (DE); Wim Van Camp, Sint-Denijs-Westrem (BE); Holger Fahnenstich, Berlin (DE); Bryan McKersie, Raleigh, NC (US); Wesley Bruce, Raleigh, NC (US); Nicole Christiansen, Berlin (DE)

(72) Inventors: Gunnar Plesch, Potsdam (DE); Astrid Blau, Stahnsdorf (DE); Michael Manfred Herold, Berlin (DE); Beate Kamlage, Berlin (DE); Birgit Wendel, Berlin (DE); Piotr Puzio, Mariakerke (Gent) (BE); Stefan Henkes, Potsdam (DE); Volker Haake, Berlin (DE); Wim Van Camp, Sint-Denijs-Westrem (BE); Holger Fahnenstich, Berlin (DE); Bryan McKersie, Raleigh, NC (US); Wesley Bruce, Raleigh, NC (US); Nicole Christiansen, Berlin (DE)

(73) Assignee: BASF Plant Science Company GmbH, Ludwigshafen (DE)

(21) Appl. No.: 13/510,783

(22) PCT Filed: Nov. 18, 2010
(86) PCT No.: PCT/EP10/06988

§ 371 Date: May 18, 2012

(30) Foreign Application Priority Data
Nov. 18, 2009 (EP) 09014472.6
Nov. 18, 2009 (EP) 09176396.1
Dec. 22, 2009 (EP) 09181052.3
Oct. 8, 2010 (EP) 10186930.3
Oct. 26, 2010 (EP) 10188863.4
Oct. 28, 2010 (EP) 10189169.5
Oct. 29, 2010 (EP) 10189358.4
Nov. 4, 2010 (EP) 10189943.3
Nov. 5, 2010 (EP) 10190115.5
Nov. 8, 2010 (EP) 10190348.2
Nov. 9, 2010 (EP) 10190441.5
Nov. 10, 2010 (EP) 10190649.3
Nov. 11, 2010 (EP) 10190780.6
Nov. 11, 2010 (EP) 10190795.4
Nov. 12, 2010 (EP) 10190934.9
Nov. 12, 2010 (EP) 10190974.5
Nov. 12, 2010 (EP) 10191021.4
Nov. 15, 2010 (EP) 10191126.1
Nov. 15, 2010 (EP) 10191238.4
Nov. 16, 2010 (EP) 10191282.2

(51) Int. Cl. C12P 1/00; C12N 15/82

(52) U.S. Cl. CPC C12P 1/00; C12N 15/82
USPC 800/278; 435/41; 536/23.1; 435/320.1; 435/419; 435/243; 435/69.1; 530/300; 530/387.1; 800/298; 435/7.1; 435/6.1

(57) ABSTRACT

The present invention relates to a process for the production of a fine chemical in a non-human organism, like a microorganism, a plant cell, a plant, a plant tissue or in one or more parts thereof. The invention furthermore relates to nucleic acid molecules, polypeptides, nucleic acid constructs, expression cassettes, vectors, antibodies, host cells, plant tissue, propagation material, harvested material, plants, microorganisms as well as agricultural compositions and to their use.

The instant application is based on and claims the benefit of prior filed European Patent Application No. 09014472.6, filed 18 Nov. 2009, prior filed European Patent Application 09176396.1, filed 18 Nov. 2009, prior filed European Patent Application 09181052.3, filed 22 Dec. 2009, prior filed European Patent Application 10189169.5, filed 28 Oct .2010, prior filed European Patent Application 10189358.4, filed 29 Oct. 2010, prior filed European Patent Application 10189943.3, filed 4 Nov. 2010, prior filed European Patent Application 10186930.3, filed 8 Oct. 2010, prior filed European Patent Application 10190115.5, filed 5 Nov. 2010, prior filed European Patent Application 10190348.2, filed 8 Nov. 2010, prior filed European Patent Application 10190441.5, filed 9 Nov. 2010, prior filed European Patent Application 10190649.3, filed 10 Nov. 2010, prior filed European Patent Application 10191021.4, filed 12 Nov. 2010, prior filed European Patent Application 10191282.2, filed 16 Nov. 2010, prior filed European Patent Application 10188863.4, filed 26 Oct. 2010, prior filed European Patent Application 10191126.1, filed 15 Nov. 2010, prior filed European Patent Application 10191238.4, filed 15 Nov. 2010, prior filed European Patent Application 10190974.5 filed 12 Nov. 2010, prior filed European Patent Application 10190780.6, filed 11 Nov. 2010, prior filed European Patent Application 10190934.9, filed 12 Nov. 2010 and prior filed European Patent Application 10190795.4, filed 11 Nov. 2010. The entire content of the above-referenced patent applications are incorporated herein by this reference.

### INCORPORATION OF SEQUENCE LISTING

The contents of the following submission on DVDs are incorporated herein by reference in its entirety: two copies of the Sequence Listing (COPY 1 and COPY 2), all on DVDs, each containing: file name: PF62758.txt, date recorded: 17 Nov. 2010.

The present invention relates to a process for the production of fine chemical in a microorganism, a plant cell, a plant or a part thereof. The invention furthermore relates to nucleic acid molecules, polypeptides, nucleic acid constructs, vectors, antibodies, host cells, plant tissue, propagation material, harvested material, plants, microorganisms as well as agricultural compositions and to their use.

Amino acids are used in many branches of industry, including the food, animal feed, cosmetics, pharmaceutical and chemical industries. Amino acids such as D,L-methionine, L-lysine or L-threonine are used in the animal feed industry. The essential amino acids valine, leucine, isoleucine, lysine, threonine, methionine, tyrosine, phenylalanine and tryptophan are particularly important for the nutrition of humans and a number of livestock species. Glycine, L-methionine and tryptophan are all used in the pharmaceutical industry. Glutamine, valine, leucine, isoleucine, histidine, arginine, proline, serine and alanine are used in the pharmaceutical and cosmetics industries. Threonine, tryptophan and D,L-methionine are widely used feed additives (Leuchtenberger, W. Amino acids—technical production and use, pp. 466-502, in Rehm et al., (Ed.) Biotechnology Vol. 6, chapter 14a, VCH Weinheim, 1996). Moreover, amino acids are suitable for the chemical industry as precursors for the synthesis of synthetic amino acids and proteins, such as N-acetylcysteine, S-carboxymethyl-L-cysteine, (S)-5-hydroxytryptophan and other subtances, as for example described in Ullmann's Encyclopedia of Industrial Chemistry, Vol. A2, pp. 57-97, VCH Weinheim, 1985.

Over one million tonnes of amino acids are currently produced annually; their market value amounts to over 2.5 billion US dollars. They are currently produced by four competing processes: Extraction from protein hydrolysates, for example L-cystine, L-leucine or L-tyrosine, chemical synthesis, for example of D,L-methionine, conversion of chemical precursors in an enzyme or cell reactor, for example L-phenylalanine, and fermentative production by growing, on an industrial scale, bacteria which have been developed to produce and secrete large amounts of the desired molecule in question.

The biosynthesis of the natural amino acids in organisms capable of producing them, for example bacteria, has been characterized thoroughly; for a review of the bacterial amino acid biosynthesis and its regulation, see H. E. Umbarger,Ann. Rev. Biochem. 47, 533 (1978).

It is known that amino acids are produced by fermentation of strains of coryneform bacteria, in particular Corynebacterium glutamicum. Due to their great importance, the production processes are constantly being improved. Process improvements can relate to measures regarding technical aspects of the fermentation, such as, for example, stirring and oxygen supply, or the nutrient media composition, such as, for example, the sugar concentration during fermentation, or to the work-up to give the product, for example by ion exchange chromatography, or to the intrinsic performance properties of the microorganism itself. Bacteria from other genera such as Escherichia or Bacillus are also used for the production of amino acids. A number of mutant strains, which produce an assortment of desirable compounds from the group of the sulfur-containing fine chemicals, have been developed via strain selection. The performance properties of said microorganisms are improved with respect to the production of a particular molecule by applying methods of mutagenesis, selection and mutant selection. Methods for the production of methionine have also been developed. In this manner, strains are obtained which are, for example, resistant to antimetabolites, such as, for example, the methionine analogues α-methyL-methionine, ethionine, norleucine, N-acetylnorleucine, S-trifluoromethyl-homocysteine, 2-amino-5-heprenoitic acid, selenomethionine, methionine sulfoximine, methoxine, 1-aminocyclopentanecarboxylic acid or which are auxotrophic for metabolites with regulatory importance and which produce sulfur-containing fine chemicals such as, for example, L-methionine. However, such processes developed for the production of methionine have the disadvantage that their yields are too low for being economically exploitable and that they are therefore not yet competitive with regard to chemical synthesis.

Zeh (Plant Physiol. 127, 792 (2001)) describes increasing the methionine content in potato plants by inhibiting threonine synthase by what is known as antisense technology. This leads to a reduced threonine synthase activity without the threonine content in the plant being reduced. This technology is highly complex; the enzymatic activity must be inhibited in a very differentiated manner since otherwise auxotrophism for the amino acid occurs and the plant will no longer grow.

Methods of recombinant DNA technology have also been used for some years to improve Corynebacterium strains producing L-amino acids by amplifying individual amino acid biosynthesis genes and investigating the effect on amino acid production.

U.S. Pat. No. 5,589,616 teaches the production of higher amounts of amino acids in plants by overexpressing a monocot storage protein in dicots. WO 96/38574, WO 97/07665, WO 97/28247, U.S. Pat. No. 4,886,878, U.S. Pat. No. 5,082,993 and U.S. Pat. No. 5,670,635 are following this approach. That means in all the aforementioned intellectual property rights different proteins or polypeptides are expressed in plants. Said proteins or polypeptides should function as amino acid sinks. Other methods for increasing amino acids such as lysine are disclosed in WO 95/15392, WO 96/38574, WO 89/11789 or WO 93/19190. In these cases special enzymes in the amino acid biosynthetic pathway such as the diphydrodipicolinic acid synthase are deregulated. This leads to an increase in the production of lysine in the different plants. Another approach to increase the level of amino acids in plants is disclosed in EP-A-0 271 408. EP-A-0 271 408 teaches the mutagenesis of plant and selection afterwards with inhibitors of certain enzymes of amino acid biosynthetic pathway. WO 2006/069610, WO 2006/092449, WO 2007/087815 and US 2007/0118916 teach inter alia the production of higher amounts of amino acids, like methionine and threonine, in microorganism or plants by expression or overexpression of selected genes.

As described above, the essential amino acids are necessary for humans and many mammals, for example for livestock. L-methionine is important as methyl group donor for the biosynthesis of, for example, choline, creatine, adrenaline, bases—especially of those of RNA and DNA, histidine, and for the transmethylation following the formation of S-adenosyL-methionine or as a sulfhydryl group donor for the formation of cysteine. Moreover, L-methionine appears to have a positive effect as pharmaceutical agent against depression.

Improving the quality of foodstuffs and animal feeds is an important task of the food-and-feed industry. This is necessary since, for example, certain amino acids, which occur in plants are limited with regard to the supply of mammals. Especially advantageous for the quality of foodstuffs and animal feeds is an as balanced as possible amino acid profile since a great excess of an amino acid above a specific concentration in the food has no further positive effect on the utilization of the food since other amino acids suddenly become limiting. A further increase in quality is only possible via addition of further amino acids, which are limiting under these conditions. The targeted addition of the limiting amino acid in the form of synthetic products must be carried out with extreme caution in order to avoid amino acid imbalance. For example, the addition of an essential amino acid stimulates protein digestion, which may cause deficiency situations for the second or third limiting amino acid, in particular. In feeding experiments, for example casein feeding experiments, the additional provision of methionine, which is limiting in casein, has revealed the fatty degeneration of liver, which could only be alleviated after the additional provision of tryptophan.

To ensure a high quality of foods and animal feeds, it is therefore necessary to add a plurality of amino acids in a balanced manner to suit the respective organism. Accordingly, there is still a great demand for new and more suitable genes, which encode enzymes or regulators, which participate in the biosynthesis of amino acids and make it possible to produce certain amino acids specifically on an industrial scale without unwanted byproducts being formed. In the selection of genes for biosynthesis or regulation two characteristics above all are particularly important. On the one hand, there is as ever a need for improved processes for obtaining the highest possible contents of amino acids and on the other hand as less as possible byproducts should be produced in the production process.

The present invention relates in paragraphs [0012.1.1.1] to [0514.1.1.1] to a process for the production of the fine chemical methionine as defined below and corrresponding embodiments as described herein as follows.

It is an object of the present invention to develop an inexpensive process for the synthesis of methionine. L-Methionine is with lysin or threonine (depending on the organism) one of the amino acids, which are most frequently limiting.

It was now found that this object is achieved by providing the process according to the invention described herein and the embodiments characterized herein as well as in the claims.

Accordingly, in a first embodiment, the invention relates to a process for the production of methionine, or, in other words, of the “fine chemical” or “fine chemical of the invention”.

The terms “fine chemical of the invention”, “fine chemical” or “the fine chemical” are used herein equally and relate in context of the paragraphs or sections [0014.1.1.1] to [0514.1.1.1] essentially to the metabolite or the metabolites indicated in column 7, application no. 1 of Tables I, II and IV in the respective line.

Further, the term “in context of any of the paragraphs [0014.1.1.1] to [0514.1.1.1]” as used herein means that for any of said paragraphs [0014.1.1.1] to [0514.1.1.1] the term “the fine chemical” is understood to follow the definition of paragraphs or sections [0014.1.1.1] and [0015.1.1.1], independently whether it refers to any other paragraph or not and whether the reference recites the term “fine chemical” in an other context.

Thus, in cases where one or more paragraphs or sections are incorporated by reference into any of the present paragraphs [0014.1.1.1] to [0514.1.1.1], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to the definition of paragraph [0014.1.1.1] and [0015.1.1.1].

Accordingly, in one embodiment, the term “fine chemical of the invention”, “fine chemical” or “the fine chemical” as used herein (alone or in combination, like in FCRP) means “methionine in context of the nucleic acid or polypeptide sequences listed in the respective same line of any one of Tables I to IV of application no. 1 and indicating in column 7 the metabolite “methionine”. In one embodiment, the term methionine or the term “fine chemical” mean in context of the paragraphs or sections [0014.1.1.1] to [0514.1.1.1] at least one chemical compound with an activity of the above mentioned methionine, respectively.

Accordingly, in one embodiment, the terms “fine chemical of the invention”, “fine chemical” or “the fine chemical” mean in context of any of the paragraphs [0014.1.1.1] to [0514.1.1.1] methionine, preferably the L-enantiomer of methionine, its salts, ester or amides in free form or bound to proteins. In a preferred embodiment , the term “the fine chemical” means methionine or its salts, in free form or bound to proteins. In a preferred embodiment, the term “the fine chemical” means the L-enantiomer of methionine. On the other hand in case “methionine” is stated it means methionine itself, its salts, ester or amides in free form or bound to proteins, preferably the L-enantiomer of methionine, its salts, ester or amides in free form or bound to proteins. In a preferred embodiment “methionine” means the L-enantiomer of methionine in free form. In another preferred embodiment “methionine” means the L-enantiomer of methionine bound to proteins.

Further, the term “in context of any of the paragraphs [0014.1.1.1] to [0514.1.1.1]” as used herein means that for any of said paragraphs [0014.1.1.1] to [0514.1.1.1] the term “the fine chemical” is understood to follow the definition of section [0014.1.1.1] or section [0015.1.1.1], independently whether it refers to any other paragraph or not and whether the reference recites the term “fine chemical” in an other context.

Thus, in cases where one or more paragraphs or sections are incorporated by reference into any of the present paragraphs [0014.1.1.1] to [0514.1.1.1], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to this definition of this paragraph [0015.1.1.1].

Further, the term “fine chemicals” as used herein relates to compositions comprising said fine chemical(s), i.e. comprising methionine, respectively.

Accordingly, the present invention relates to a process for the production of methionine, which comprises
(a) increasing or generating one or more activities selected from the group consisting of 49747384_SOYBEAN-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of At1 g09680-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of DNA-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of bi-functional aspartokinase/homoserine dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of At2g45420-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of adenosine kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of auxin response factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of monthiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of At4g32480-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of zinc finger protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of malic enzyme in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of beta-hydroxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of 2-oxoglutarate dehydrogenase E1 subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism. Accordingly, the present invention relates to a process for the production of methionine, which comprises
(a) increasing or generating one or more activities selected from the group consisting of homoserine dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of peptidyl-prolyl cis-trans isomerase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of b0012-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of acetolactate synthase small subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of serine protease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of 2,3-dihydroxyphenylpropionate 1,2-dioxygenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of ATP-binding component of a transport system in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of membrane transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of threonine aldolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of major facilitator superfamily transporter protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of b1003-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of b1522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of L-serine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of serine/threonine-protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of b2032-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of uridine/cytidine kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of b2345-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of b2513-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of b2673-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of arginine exporter protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of b3246-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of acetyl CoA carboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of b3346-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of valine-pyruvate transaminase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of dihydroxyacid dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of b3817-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of threonine efflux protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of b4029-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of 5′-nucleotidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of glutathione S-transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of coproporphyrinogen Ill oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of Photosystem I reaction center subunit XI in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of phosphoadenosine phosphosulfate reductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of threonine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of Sec-independent protein translocase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of homocitrate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of cystathionine -lyase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of cyclin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of F-box protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of yh1013c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of (DL)-glycerol-3-phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of glycogenin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of cystathionine gamma-synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of homoserine dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of yml084w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of yol160w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating one or more activities selected from the group consisting of yor392w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

An embodiment of the present invention relates to a process for the production of methionine, which comprises
(a1) increasing or generating the activity of a polypeptide comprising a polypeptide as depicted in a respective line in column 5 or 8 of Table II, application no. 1, or a homolog or a fragment thereof, a consensus sequence or at least one polypeptide motif as depicted in the respective line in column 8 of Table IV, application no. 1; or
(a2) increasing or generating the activity of an expression product of one or more nucleic acid molecule(s) comprising a polynucleotide as depicted in the respective line in column 5 or 8 of Table I, application no. 1, preferably the coding region thereof, or a homolog or a fragment thereof; or
(a3) increasing or generating the activity of a functional equivalent of (a1) or (a2); in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of methionine or a composition comprising methionine in said non-human organism or in the culture medium surrounding said non-human organism.

Accordingly, the increase or generation of one or more said activities is for example conferred by one or more expression products of said nucleic acid molecule, e.g. proteins. Accordingly, in the present invention described above, the increase or generation of one or more said activities is for example conferred by one or more protein(s) each comprising a polypeptide selected from the group as depicted in Table II, application no. 1, column 5 and 8, or a homolog or a fragment thereof.

The process of the invention comprises in one embodiment the following steps:
(i) increasing or generating of the expression of; and/or
(ii) increasing or generating the expression of an expression product of; and/or
(iii) increasing or generating one or more activities of an expression product encoded by;

at least one nucleic acid molecule (in the following “Fine Chemical Related Protein (FCRP)”encoding gene or “FCRP”-gene) comprising a nucleic acid molecule selected from the group consisting of:
(a) a nucleic acid molecule encoding the polypeptide shown in column 5 or 8 of Table II, preferably Table II B, application no. 1, or a homolog or a fragment thereof;
(b) a nucleic acid molecule shown in column 5 or 8 of Table I, preferably Table I B, application no. 1, or a homolog or a fragment thereof (preferably the coding region thereof);
(c) a nucleic acid molecule, which, as a result of the degeneracy of the genetic code, can be derived from a polypeptide sequence depicted in column 5 or 8 of Table II, preferably Table II B, application no. 1;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 1, or the coding region thereof;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d),
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 1;
(i) a nucleic acid molecule encoding a polypeptide having the activity represented by a respective protein as depicted in column 5 of Table II, application no. 1;
(j) a nucleic acid molecule which comprises a polynucleotide, which is obtained by amplifying a cDNA library or a genomic library using the primers as depicted in column 8 of Table III, application no. 1; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j); or a nucleic acid molecule comprising a sequence which is complementary thereto.

(All the references to Tables I to IV are references to the corresponding lines of Tables I to IV.)

The term “increasing or generating of the expression of” may also be interchanged by the term “overexpressing”.

In a preferred embodiment thereof said nucleic acid molecule encodes a polypeptide which has the activity of the polypeptide represented by a protein comprising a polypeptide as depicted in the corresponding hit in column 5 of Table II, application no. 1.

In another preferred embodiment thereof said nucleic acid molecule confers a generation or an increase of the respective fine chemical in a non-human organism or a part thereof as compared to a corresponding wild-type non-human organism or part thereof.

In another preferred embodiment thereof said nucleic acid molecule encodes a polypeptide which has the activity of the polypeptide represented by a protein as depicted in the corresponding hit in column 5 of Table II, application no. 1, and confers a generation or an increase of the respective fine chemical in a non-human organism or a part thereof as compared to a corresponding wild-type non-human organism or part thereof.

Accordingly, the genes of the present invention or used in accordance with the present invention, which respectively encode a protein having an activity of (DL)-glycerol-3-phosphatase, 2,3-dihydroxyphenylpropionate 1,2-dioxygenase, 2-oxoglutarate dehydrogenase E1 subunit, 49747384_SOYBEAN-protein, 5′-nucleotidase, acetolactate synthase small subunit, acetyl CoA carboxylase, adenosine kinase, arginine exporter protein, At1g09680-protein, At2g45420-protein, At4g32480-protein, ATP-binding component of a transport system, auxin response factor, b0012-protein, b1003-protein, b1522-protein, b2032-protein, b2345-protein, b2513-protein, b2673-protein, b3246-protein, b3346-protein, b3817-protein, b4029-protein, beta-hydroxylase, bifunctional aspartokinase/homoserine dehydrogenase, calcium-dependent protein kinase, coproporphyrinogen III oxidase, cyclin, cystathionine gamma-synthase, cystathionine-lyase, dihydroxyacid dehydratase, DNA-binding protein, F-box protein, glutaredoxin, glutathione S-transferase, glycogenin, homocitrate synthase, homoserine dehydrogenase, hydrolase, L-serine dehydratase, major facilitator superfamily transporter protein, malic enzyme, membrane transport protein, monothiol glutaredoxin, monthiol glutaredoxin, oxidoreductase, peptidyl-prolyl cis-trans isomerase, phosphoadenosine phosphosulfate reductase , Photosystem I reaction center subunit XI, protein kinase, protein phosphatase, pyruvate kinase, Sec-independent protein translocase subunit, serine protease, serine/threonine-protein phosphatase, threonine aldolase, threonine dehydratase, threonine efflux protein, transcription factor, transport protein, uridine/cytidine kinase, valine-pyruvate transaminase, yhl013c-protein, yml084w-protein, yol160w-protein, yor392w-protein, or zinc finger protein, which respectively encode a protein comprising a polypeptide encoded by a nucleic acid sequence as shown in Table I, application no. 1, column 5 or 8, (preferably the coding region thereof), or a homolog or a fragment thereof, which respectively encode a protein comprising a polypeptide as depicted in Table II, application no. 1, column 5 or 8, or a homolg or a fragment thereof, and/or which respectively can be amplified with the primer set shown in Table III, application no. 1, column 8, are also referred to as “FCRP genes”.

Proteins or polypeptides encoded by “FCRP-genes” are referred to as “Fine Chemical Related Proteins” or “FCRP”. For the purposes of the description of the present invention, the respective protein having an activity of (DL)-glycerol-3-phosphatase, 2,3-dihydroxyphenylpropionate 1,2-dioxygenase, 2-oxoglutarate dehydrogenase E1subunit, 49747384_SOYBEAN-protein, 5′-nucleotidase, acetolactate synthase small subunit, acetyl CoA carboxylase, adenosine kinase, arginine exporter protein, At1g09680-protein, At2g45420-protein, At4g32480-protein, ATP-binding component of a transport system, auxin response factor, b0012-protein, b1003-protein, b1522-protein, b2032-protein, b2345-protein, b2513-protein, b2673-protein, b3246-protein, b3346-protein, b3817-protein, b4029-protein, beta-hydroxylase, bifunctional aspartokinase/homoserine dehydrogenase, calcium-dependent protein kinase, coproporphyrinogen III oxidase, cyclin, cystathionine gamma-synthase, cystathionine-lyase, dihydroxyacid dehydratase, DNA-binding protein, F-box protein, glutaredoxin, glutathione S-transferase, glycogenin, homocitrate synthase, homoserine dehydrogenase, hydrolase, L-serine dehydratase, major facilitator superfamily transporter protein, malic enzyme, membrane transport protein, monothiol glutaredoxin, monthiol glutaredoxin, oxidoreductase, peptidyl-prolyl cistrans isomerase, phosphoadenosine phosphosulfate reductase , Photosystem I reaction center subunit XI, protein kinase, protein phosphatase, pyruvate kinase, Sec-independent protein translocase subunit, serine protease, serine/threonine-protein phosphatase, threonine aldolase, threonine dehydratase, threonine efflux protein, transcription factor, transport protein, uridine/cytidine kinase, valine-pyruvate transaminase, yh1013c-protein, yml084w-protein, yol160w-protein, yor392w-protein, or zinc finger protein, the respective protein comprising a polypeptide encoded by one or more respective nucleic acid sequences as shown in Table I, application no. 1, column 5 or 8, (preferably the coding region thereof), or a homolog or fragment thereof, the respective protein comprising a respective polypeptide as depicted in Table II, application no. 1, column 5 or 8, or a homolog or fragment thereof, the respective protein comprising a sequence corresponding to the consensus sequence as shown in Table IV, application no. 1, column 8, and/or the respective protein comprising at least one polypeptide motif as shown in Table IV, application no. 1, column 8 is also referred to as Fine Chemical Related Protein” or “FCRP”.

Thus, in one embodiment, the present invention provides a process of the production of methionine, by increasing or generating one or more activities, especially selected from the group consisting of (DL)-glycerol-3-phosphatase, 2,3-dihydroxyphenylpropionate 1,2-dioxygenase, 2-oxoglutarate dehydrogenase E1 subunit, 49747384_SOYBEAN-protein, 5′-nucleotidase, acetolactate synthase small subunit, acetyl CoA carboxylase, adenosine kinase, arginine exporter protein, At1g09680-protein, At2g45420-protein, At4g32480-protein, ATP-binding component of a transport system, auxin response factor, b0012-protein, b1003-protein, b1522-protein, b2032-protein, b2345-protein, b2513-protein, b2673-protein, b3246-protein, b3346-protein, b3817-protein, b4029-protein, beta-hydroxylase, bifunctional aspartokinase homoserine dehydrogenase, calcium-dependent protein kinase, coproporphyrinogen III oxidase, cyclin, cystathionine gamma-synthase, cystathionine-lyase, dihydroxyacid dehydratase, DNA-binding protein, F-box protein, glutaredoxin, glutathione S-transferase, glycogenin, homocitrate synthase, homoserine dehydrogenase, hydrolase, L-serine dehydratase, major facilitator superfamily transporter protein, malic enzyme, membrane transport protein, monothiol glutaredoxin, monthiol glutaredoxin, oxidoreductase, peptidyl-prolyl cis-trans isomerase, phosphoadenosine phosphosulfate reductase , Photosystem I reaction center subunit XI, protein kinase, protein phosphatase, pyruvate kinase, Sec-independent protein translocase subunit, serine protease, serine/threonine-protein phosphatase, threonine aldolase, threonine dehydratase, threonine efflux protein, transcription factor, transport protein, uridine/cytidine kinase, valine-pyruvate transaminase, yhl013c-protein, yml084w-protein, yol160w-protein, yor392w-protein, and zinc finger protein, which is conferred by one or more FCRPs or the gene product of one or more FCRP-genes, for example by the gene product of a nucleic acid sequences comprising a polynucleotide selected from the group as shown in Table I, application no. 1, column 5 or 8, (preferably by the coding region thereof), or a homolog or a fragment thereof, e.g. or by one or more proteins each comprising a polypeptide encoded by one or more nucleic acid sequences selected from the group as shown in Table I, application no. 1, column 5 or 8, (preferably by the coding region thereof), or a homolog or a fragment thereof, or by one or more protein(s) each comprising a polypeptide selected from the group as depicted in Table II, application no. 1, column 5 and 8, or a homolog thereof, or a protein comprising a sequence corresponding to the consensus sequence or comprising at least one polypeptide motif as shown in Table IV, application no. 1, column 8.

As mentioned, the process for the production of the fine chemical according to the present invention, in particular showing a generation or an increase of the respective fine chemical in a non-human organism or a part thereof as compared to a corresponding wild-type non-human organism or part thereof, can be mediated by one or more FCRP-genes or FCPRs.

In an embodiment, the process comprises increasing or generating the activity of one or more polypeptides having said activity, e.g. by generating or increasing the amount and/or specific activity in the cell or a compartment of a cell of one of more FCRP, especially selected from the group consisting of (DL)-glycerol-3-phosphatase, 2,3-dihydroxyphenylpropionate 1,2-dioxygenase, 2-oxoglutarate dehydrogenase E1 subunit, 49747384_SOYBEAN-protein, 5′-nucleotidase, acetolactate synthase small subunit, acetyl CoA carboxylase, adenosine kinase, arginine exporter protein, At1g09680-protein, At2g45420-protein, At4g32480-protein, ATP-binding component of a transport system, auxin response factor, b0012-protein, b1003-protein, b1522-protein, b2032-protein, b2345-protein, b2513-protein, b2673-protein, b3246-protein, b3346-protein, b3817-protein, b4029-protein, beta-hydroxylase, bifunctional aspartokinase/homoserine dehydrogenase, calcium-dependent protein kinase, coproporphyrinogen III oxidase, cyclin, cystathionine gamma-synthase, cystathionine-lyase, dihydroxyacid dehydratase, DNA-binding protein, F-box protein, glutaredoxin, glutathione S-transferase, glycogenin, homocitrate synthase, homoserine dehydrogenase, hydrolase, L-serine dehydratase, major facilitator superfamily transporter protein, malic enzyme, membrane transport protein, monothiol glutaredoxin, monthiol glutaredoxin, oxidoreductase, peptidyl-prolyl cistrans isomerase, phosphoadenosine phosphosulfate reductase , Photosystem I reaction center subunit XI, protein kinase, protein phosphatase, pyruvate kinase, Sec-independent protein translocase subunit, serine protease, serine/threonine-protein phosphatase, threonine aldolase, threonine dehydratase, threonine efflux protein, transcription factor, transport protein, uridine/cytidine kinase, valine-pyruvate transaminase, yhl013c-protein, yml084w-protein, yol160w-protein, yor392w-protein, and zinc finger protein, for example of the respective polypeptide as depicted in Table II, application no. 1, column 5 and 8, or a homolog or a fragment thereof, or the respective polypeptide comprising a sequence corresponding to the consensus sequences as shown in Table IV, application no. 1, column 8, or the respective polypeptide comprising at least one polypeptide motif as depicted in Table IV, application no. 1, column 8.

A further embodiment of the present invention relates to a process for the production of methionine, which comprises
(a) increasing or generating the activiy of a 49747384_SOYBEAN-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a At1g09680-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a DNA-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a bifunctional aspartokinase/homoserine dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a At2g45420-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a adenosine kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a auxin response factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a monthiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a At4g32480-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a zinc finger protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a malic enzyme non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a beta-hydroxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a 2-oxoglutarate dehydrogenase E1 subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a homoserine dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a peptidyl-prolyl cis-trans isomerase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a b0012-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a acetolactate synthase small subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a serine protease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a 2,3-dihydroxyphenylpropionate 1,2-dioxygenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a ATP-binding component of a transport system non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a membrane transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a threonine aldolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a major facilitator superfamily transporter protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a b1003-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a b1522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a L-serine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a serine/threonine-protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a b2032-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a uridine/cytidine kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a b2345-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a b2513-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a b2673-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a arginine exporter protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a b3246-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a acetyl CoA carboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a b3346-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a valine-pyruvate transaminase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a dihydroxyacid dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a b3817-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a threonine efflux protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a b4029-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a 5′-nucleotidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a glutathione S-transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a coproporphyrinogen Ill oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a Photosystem I reaction center subunit XI non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a phosphoadenosine phosphosulfate reductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a threonine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a Sec-independent protein translocase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a homocitrate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a cystathionine-lyase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a cyclin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a F-box protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a yhl013c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a (DL)-glycerol-3-phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a glycogenin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a cystathionine gamma-synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a homoserine dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a yml084w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a yol160w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activiy of a yor392w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating the activity of a polypeptide comprising a polypeptide as depicted in the respective line in column 5 or 8 of Table II, application no. 1, or a homolog or a fragment thereof, a consensus sequence or at least one polypeptide motif as depicted in the respective line in column 8 of Table IV, application no. 1; or
   increasing or generating the activity of an expression product of one or more nucleic acid molecule(s) comprising a polynucleotide as depicted in the respective line in column 5 or 8 of Table I, application no. 1, preferably the coding region thereof, or a homolog or a fragment thereof;
   non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of methionine, or a composition comprising methionine in said non-human organism or in the culture medium surrounding said non-human organism.

A further embodiment of the present invention relates to a process for the production of methionine, which comprises
(a) increasing or generating one or more activities selected from the group consisting of 49747384_SOYBEAN-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of At1g09680-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of DNA-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of bi-functional aspartokinase/homoserine dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of At2g45420-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of adenosine kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of auxin response factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of monthiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of At4g32480-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of zinc finger protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of malic enzyme in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of betahydroxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of 2-oxoglutarate dehydrogenase E1 subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of homoserine dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of peptidyl-prolyl cis-trans isomerase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of b0012-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of acetolactate synthase small subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of serine protease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of 2,3-dihydroxyphenylpropionate 1,2-dioxygenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of ATP-binding component of a transport system in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of membrane transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of threonine aldolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of major facilitator superfamily transporter protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of b1003-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of b1522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of L-serine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of serine/threonine-protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of b2032-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of uridine/cytidine kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of b2345-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of b2513-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of b2673-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of arginine exporter protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of b3246-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of acetyl CoA carboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of b3346-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of valine-pyruvate transaminase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of dihydroxyacid dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of b3817-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of threonine efflux protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of b4029-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of 5′-nucleotidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of glutathione S-transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of coproporphyrinogen III oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of Photosystem I reaction center subunit XI in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of phosphoadenosine phosphosulfate reductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of threonine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of Sec-independent protein translocase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of homocitrate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of cystathionine-lyase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of cyclin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of F-box protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of yhl013c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of (DL)-glycerol-3-phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of glycogenin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of cystathionine gamma-synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of homoserine dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of yml084w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of yol160w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a) increasing or generating one or more activities selected from the group consisting of yor392w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

A further embodiment of the present invention relates to a process for the production of methionine, which comprises
(a1) increasing or generating the activity of a polypeptide comprising a polypeptide as depicted in the respective line in column 5 or 8 of Table II, application no. 1, or a homolog or fragment thereof, a consensus sequence or at least one polypeptide motif as depicted in column 8 of Table IV, application no. 1; or
   increasing or generating the activity of an expression product of one or more nucleic acid molecule(s) comprising a polynucleotide as depicted in the respective line in column 5 or 8 of Table I, application no. 1, preferably the coding region thereof, or a homolog or a fragment thereof;
   in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 1, or a homolog or a fragment thereof, a consensus sequence or at least one polypeptide motif as depicted in the respective line in column 8 of Table IV, application no. 1, which is joined to a transit peptide; or
   increasing or generating the activity of an expression product of one or more nucleic acid molecule(s) comprising a polynucleotide as depicted in the respective line in column 5 or 8 of Table I, application no. 1, preferably the coding region thereof, or a homolog or a fragment thereof, which is joined to a nucleic acid sequence encoding an organelle localization sequence, preferably a plastid or a mitochondrion locatization sequence, especially a plastid localization sequence;
   in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 1, or a homolog or a fragment thereof, a consensus sequence or at least one polypeptide motif as depicted in the respective line in column 8 of Table IV, application no. 1; or
   increasing or generating the activity of an expression product of one or more nucleic acid molecule(s) comprising a polynucleotide as depicted in the respective line in column 5 or 8 of Table I, application no. 1, preferably the coding region thereof, or a homolog or a fragment thereof;
   in an organelle, preferably in plastids or imitochondra, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of methionine, or a composition comprising methionine in said non-human organism or in the culture medium surrounding said non-human organism.

Accordingly, the present invention relates to a process for the production of methionine, which comprises
(a) increasing or generating the activity of a 49747384_SOYBEAN-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a At1g09680-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a DNA-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a bifunctional aspartokinase/homoserine dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a At2g45420-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a adenosine kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a auxin response factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a monthiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a At4g32480-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a zinc finger protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a malic enzyme in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a beta-hydroxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a 2-oxoglutarate dehydrogenase E1 subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a homoserine dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a peptidyl-prolyl cis-trans isomerase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a b0012-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a acetolactate synthase small subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a serine protease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a 2,3-dihydroxyphenylpropionate 1,2-dioxygenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a ATP-binding component of a transport system in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a membrane transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a threonine aldolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a major facilitator superfamily transporter protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a b1003-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a b1522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a L-serine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a serine/threonine-protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a b2032-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a uridine/cytidine kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a b2345-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a b2513-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a b2673-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a arginine exporter protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.
   Accordingly, the present invention relates to a process for the production of methionine, which comprises
(a) increasing or generating the activity of a b3246-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a acetyl CoA carboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a b3346-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a valine-pyruvate transaminase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a dihydroxyacid dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a b3817-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a threonine efflux protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a b4029-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a 5′-nucleotidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a glutathione S-transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a coproporphyrinogen III oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a Photosystem I reaction center subunit XI in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a phosphoadenosine phosphosulfate reductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a threonine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a Sec-independent protein translocase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a homocitrate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a cystathionine-lyase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a cyclin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a F-box protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a yhl013c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a (DL)-glycerol-3-phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a glycogenin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a cystathionine gamma-synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a homoserine dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a yml084w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a yol160w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a yor392w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Accordingly, the present invention relates to a process for the production of methionine, which comprises
(a) increasing or generating the activity of a polypeptide comprising a polypeptide as depicted in the respective line in column 5 or 8 of Table II, application no. 1, or a homolog or a fragment thereof, a consensus sequence or at least one polypeptide motif as depicted in the respective line in column 8 of Table IV, application no. 1; or
   increasing or generating the activity of an expression product of one or more nucleic acid molecule(s) comprising a polynucleotide as depicted in the respective line in column 5 or 8 of Table I, application no. 1, preferably the coding region thereof, or a homolog or a fragment thereof;
   in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of methionine, or a composition comprising methionine in said non-human organism or in the culture medium surrounding said non-human organism.

In a further embodiment the activity of the polypeptide comprising a polypeptide as depicted in the respective line in column 5 or 8 of Table II, application no. 1, or a homolog or a fragment thereof, a consensus sequence or at least one polypeptide motif as depicted in the respective line in column 8 of Table IV, application no. 1, is increased or generated non-targeted in the above-mentioned process in a microorganism or plant or a part thereof.

In a further embodiment said polypeptide has the activity of the respective polypeptide represented by a protein comprising a polypeptide as depitcted in the respective line in column 5 of Table II, application no. 1.

In a further embodiment the activity of the expression product of one or more nucleic acid molecule(s) comprising a polynucleotide as depicted in the respective line in column 5 or 8 of Table I, application no. 1, preferably the coding region thereof, or a homolog or a fragment thereof, is increased or generated non-targeted in the above-mentioned process in a microorganism or plant or a part thereof.

In a further embodiment the activity of the polypeptide comprising a polypeptide as depicted in the respective line in column 5 or 8 of Table II, application no. 1, or a homolog or a fragment thereof, a consensus sequence or at least one polypeptide motif as depicted in the respective line in column 8 of Table IV, application no. 1, is increased or generated in the above-mentioned process in an organelle preferably in plastids or mitochondria, especially in plastids, of a microorganism or plant.

In a further embodiment said polypeptide has the activity of the respective polypeptide represented by a protein comprising a polypeptide as depitcted in the respective line in column 5 of Table II, application no. 1.

In a further embodiment the activity of the expression product of one or more nucleic acid molecule(s) comprising a polynucleotide as depicted in the respective line in column 5 or 8 of Table I, application no. 1, preferably the coding region thereof, or a homolog or a fragment thereof, is increased or generated in the above-mentioned process in an organelle, preferably in plasids or mitochondria, especially in plastids, of a microorganism or plant.

In a further embodiment the activity of the polypeptide comprising a polypeptide as depicted in the respective line in column 5 or 8 of Table II, application no. 1, or a homolog or a fragment thereof, a consensus sequence or at least one polypeptide motif as depicted in the respective line in column 8 of Table IV, application no. 1, is increased or generated in the above-mentioned process in the cyctosol of a cell, of a microorganism or plant.

In a further embodiment said polypeptide has the activity of the respective polypeptide represented by a protein comprising a polypeptide as depitcted in the respective line in column 5 of Table II, application no. 1.

In a further embodiment the activity of the expression product of one or more nucleic acid molecule(s) comprising a polynucleotide as depicted in the respective line in column 5 or 8 of Table I, application no. 1, preferably the coding region thereof, or a homolog or a fragment thereof, is increased or generated in the above-mentioned process in the cytosol of a cell, of a microorganism or plant.

In a further embodiment of the present invention the process further comprises the step of recovering the fine chemical, which is synthesized by the organism from the organism and/or from the culture medium used for the growth or maintenance of the organism.

For the purposes of the present invention, as a rule the plural is intended to encompass the singular and vice versa, unless otherwise specified.

Comprises/comprising and grammatical variations thereof when used in this specification are to be taken to specify the presence of stated features, integers, steps or components or groups thereof, but not to preclude the presence or addition of one or more other features, integers, steps, components or groups thereof.

The term “Table I” used in this specification is to be taken to specify the content of Table I A and Table I B. The term “Table II” used in this specification is to be taken to specify the content of Table II A and Table II B. The term “Table I A” used in this specification is to be taken to specify the content of Table I A. The term “Table I B” used in this specification is to be taken to specify the content of Table I B. The term “Table II A” used in this specification is to be taken to specify the content of Table II A. The term “Table II B” used in this specification is to be taken to specify the content of Table II B. In one preferred embodiment, the term “Table I” means Table I B. In one preferred embodiment, the term “Table II” means Table II B. In a line of Table I related nucleic acid molecules are listed. In column 3 the locus name, often also referred to as gene name, is given, in column 5 the lead sequence ID No. thereto and in column 8 the sequence ID No. of homologues thereof. In the corresponding line of Table II the respective polypeptides are listed. In column 3 the protein name is given (which is according to the common understanding of the skilled person in the art usually used for the gene as well as the polypeptide and therefore identical with the gene name/locus name), in column 5 the (corresonding) lead sequence ID No. thereto and in column 8 the (corresponding) sequence ID No. of homologues thereof.

In Tables I and II in column 4 information is given from which organism the lead sequence according to column 5 has been identified, in column 7 information is given which fine chemical is generated or increased, and in an especial embodiment in column 6 information is given about non-targeted expression or expression in plastids or mitochon dria.

Tables III and IV are arranged accordingly whereby in column 8 of Table III primers are listed which can be used to amplify the sequence of the corresponding lead sequence indicated in column 5 of the same line and whereby in column 8 of Table IV consensus and pattern sequences are listed which are shared by the lead sequence as indicated in column 5 of the same line and their homologs listed in the same line in Table II column 8. How the consensus and pattern sequences are determined is described lateron in the application in more detail.

The terms “increase”, “raise”, “extend”, “enhance”, “improve” and “amplify” as well as the grammatical versions thereof relate to a corresponding change of a property in a non-human organism, a part of an organism such as a tissue, seed, root, leave, flower, pollen etc. or in a cell and are interchangeable. Preferably, the overall activity in the volume is increased or enhanced in cases if the increase or enhancement is related to the increase or enhancement of an activity of a gene product, independent whether the amount of gene product or the specific activity of the gene product or both is increased or enhanced or whether the amount, stability or translation efficacy of the nucleic acid sequence or gene encoding for the gene product is increased or enhanced.

Under “change of a property” it is understood that the activity, expression level or amount of a gene product or the metabolite content is changed in a specific volume relative to a corresponding volume of a control, reference or wild type, including the de novo creation of the activity or expression.

The term “increase” may be directed to a change of said property in the subject of the present invention or only in a part thereof, for example, the change can be found in a compartment of a cell, like an organelle, or in a part of an non-human organism, like plant tissue, plant seed, plant root, pollen, leave, flower etc. but is not detectable in the overall subject, i.e. complete cell or plant, if tested.

Accordingly, the term “increase” means that the specific activity of a polypeptide or the amount of a compound or of a metabolite, e.g. of a polypeptide, a nucleic acid molecule or an encoding mRNA or DNA or the fine chemical, can be increased in a volume.

The term “increase” includes that a compound or an activity is introduced into a cell or a subcellular compartment or organelle de novo or that the compound or the activity has not been detectable before, in other words it is “generated”.

Accordingly, in the following, the term “increasing” also comprises the term “generating” or “stimulating”. The increased activity manifests itself in an increase of the fine chemical.

The terms “wild type”, “control” or “reference” are exchangeable and can be a cell or a part of a non-human organism such as an organelle like a chloroplast or a tissue, or a non-human organism, in particular a microorganism or a plant, which was not modified or treated according to the herein described process according to the invention. Accordingly, the cell or a part of a non-human organism such as an organelle like a chloroplast or a tissue, or an organism, in particular a microorganism or a plant used as wild type, control or reference corresponds to the cell, non-human organism, microorganism, plant, or a part thereof, as much as possible and is in any other property but in the result of the process of the invention as identical to the subject matter of the invention as possible. Thus, the wild type, control or reference is treated identically or as identical as possible, saying that only conditions or properties might be different which do not influence the quality of the tested property.

Preferably, any comparison is carried out under analogous conditions. The term “analogous conditions” means that all conditions such as, for example, culture or growing conditions (such as soil, nutrient, water content of the soil, temperature, humidity or surrounding air), or assay conditions (such as buffer composition, temperature, substrates, pathogen strain, concentrations and the like) are kept identical between the experiments to be compared.

The “reference”, “control” or “wild type” is preferably a subject, e.g. an organelle, a cell, a tissue, an organism, in particular a microorganism or a plant, which was not modified or treated according to the herein described process of the invention and is in any other property as similar to the subject matter of the invention as possible. The reference, control or wild type is in its genome, transcriptome, proteome or metabolome as similar as possible to the subject of the present invention. Preferably, the term “reference-”, “control-” or “wild type-”-organelle, -cell, -tissue or -organism, in particular a microorganism or a plant, relates to an organelle, cell, tissue or organism, in particular a microorganism or a plant, which is nearly genetically identical to the organelle, cell, tissue or organism, in particular a microorganism or plant, or a part thereof, of the present invention, preferably 95%, more preferred are 98%, even more preferred are 99.00%, in particular 99.10%, 99.30%, 99.50%, 99.70%, 99.90%, 99.99%, 99.999% or more. Most preferable the “reference”, “control”, or “wild type” is a subject, e.g. an organelle, a cell, a tissue, an organism, in particular a microorganism or a plant, which is genetically identical to the organism, in particular plant, cell, a tissue or organelle used according to the process of the invention except that the responsible or activity conferring nucleic acid molecules or the gene product encoded by them are amended, manipulated, exchanged or introduced according to the inventive process.

In case, a control, reference or wild type differing from the subject of the present invention only by not being subject of the process of the invention can not be provided, a control, reference or wild type can be a non-human organism in which the cause for the modulation of an activity conferring the generation/increase of the fine chemical or expression of the nucleic acid molecule of the invention as described herein has been switched back or off, e.g. by knocking out the expression of responsible gene product, e.g. by antisense inhibition, by inactivation of an activator or agonist, by activation of an inhibitor or antagonist, by inhibition through adding inhibitory antibodies, by adding active compounds as e.g. hormones, by introducing negative dominant mutants, etc. A gene production can for example be knocked out by introducing inactivating point mutations, which lead to an enzymatic activity inhibition or a destabilization or an inhibition of the ability to bind to cofactors etc.

Accordingly, preferred reference subject is the starting subject of the present process of the invention. Preferably, the reference and the subject matter of the invention are compared after standardization and normalization, e.g. to the amount of total RNA, DNA, or protein or activity or expression of reference genes, like housekeeping genes, such as ubiquitin, actin or ribosomal proteins.

In accordance with the invention, the term “non-human organism” as understood herein relates always to an organism with the exception of a human being, in particular to an animal or a plant or a microorganism. Further, the term “animal” as understood herein relates always to a non-human animal. Preferably the term “non-human organism” shall mean a microorganism, in particular those containing plastids, such as algae, or a plant or the like.

As used herein, “plant” is meant to include not only a whole plant but also a part thereof i.e., one or more cells, tissues, including for example, leaves, stems, shoots, roots, flowers, fruits, seeds and pollen.

The term “organelle” according to the invention shall mean for example “mitochondrion” or “plastid”. The term “plastid” according to the invention is intended to include various forms of plastids including proplastids, chloroplasts, chromoplasts, gerontoplasts, leucoplasts, amyloplasts, elaioplasts and etioplasts, preferably chloroplasts. They all have as a common ancestor the aforementioned proplasts.

Unless otherwise specified, the terms “polynucleotides”, “nucleic acid” and “nucleic acid molecule” are interchangeable in the present context. Unless otherwise specified, the terms “peptide”, “polypeptide” and “protein” are interchangeably in the present context. The term “sequence” may relate to polynucleotides, nucleic acids, nucleic acid molecules, peptides, polypeptides and proteins, depending on the context in which the term “sequence” is used. The terms “gene(s)”, “polynucleotide”, “nucleic acid sequence”, “nucleotide sequence”, or “nucleic acid molecule(s)” as used herein refer to a polymeric form of nucleotides of any length, either ribonucleotides or deoxyribonucleotides. The terms refer only to the primary structure of the molecule.

Thus, the terms “gene(s)”, “polynucleotide”, “nucleic acid sequence”, “nucleotide sequence”, or “nucleic acid molecule(s)” as used herein include double- and single-stranded DNA and/or RNA. They also include known types of modifications, for example, methylation, “caps”, substitutions of one or more of the naturally occurring nucleotides with an analog. Preferably, the DNA or RNA sequence comprises a coding sequence encoding the herein defined polypeptide.

A “coding sequence” is a nucleotide sequence, which is transcribed into a RNA, e.g. a regulatory RNA, such as a miRNA, a ta-siRNA, cosuppression molecule, an RNAi, a ribozyme, etc. or preferably into a mRNA which is translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleotide sequences or genomic DNA, while introns may be present as well under certain circumstances. The terms “coding sequence” and “coding region” are interchangeable in the present context.

As used in the present context a nucleic acid molecule may also encompass the untranslated sequence located at the 3′ and/or at the 5′ end of the coding gene region, for example at least 500, preferably 200, especially preferably 100, nucleotides of the sequence upstream of the 5′ end of the coding region and/or at least 100, preferably 50, especially preferably 20, nucleotides of the sequence downstream of the 3′ end of the coding gene region. In the event for example the antisense, RNAi, snRNA, dsRNA, siRNA, miRNA, ta-siRNA, cosuppression molecule, ribozyme etc. technology is used coding regions as well as the 5′- and/or 3′-regions can advantageously be used.

However, it is often advantageous only to choose the coding region of a nucleic acid sequence for cloning and expression purposes. Except as not otherwise specified in the sequence listing according to WI PO Standard 25 for a respective CDS usually the coding region of a nucleic acid molecule is depicted at numeric identifier <222>, whereby the coding region starts at the position given by the first number (given in brackets) and ends at the position given by the second number (given in brackets) and thereafter under numeric identifier <400>the respective sequence is disclosed.

Unless otherwise specified, the term “polypeptide” refers to a polymer of amino acids (amino acid sequence) and does not refer to a specific length of the molecule. Thus, peptides and oligopeptides are included within the definition of polypeptide. This term does also refer to or include post-translational modifications of the polypeptide, for example, glycosylations, acetylations, phosphorylations and the like. Included within the definition are, for example, polypeptides containing one or more analogs of an amino acid (including, for example, unnatural amino acids, etc.), polypeptides with substituted linkages, as well as other modifications known in the art, both naturally occurring and non-naturally occurring. The terms “protein” and “polypeptide” used in this application are interchangeable.

The term “recovering” means the isolation of the fine chemical in different purities, that means on the one hand harvesting of the biological material, which contains the fine chemical without further purification and on the other hand purities of the fine chemical between 5% and 100% purity, preferred purities are in the range of 10% and 99%. In one embodiment, the purities are at least 20%, 30%, 40%, 50%, 60%, 70%, 80%, 90%, 95% or 99%.

A “transit peptide” is an amino acid sequence, whose encoding nucleic acid sequence is translated together with the corresponding structural gene. That means the transit peptide is an integral part of the translated protein and forms an amino terminal extension of the protein. Both are translated as so called “preprotein”. In general the transit peptide is cleaved off from the preprotein during or just after import of the protein into the correct cell organelle such as a plastid or mitochondrion to yield the mature protein. The transit peptide ensures correct localization of the mature protein by facilitating the transport of proteins through intracellular membranes. In principle the nucleic acid sequence encoding a transit peptide can be isolated from every organism such as microorganisms such as algae or plants containing plastids, preferably chloroplasts, or mitochondria. Preferred nucleic acid sequences encoding a transit peptide are derived from a nucleic acid sequence encoding a protein finally resided in the plastid or mitochondria, respectively, and stemming from an organism selected from the group consisting of the genera:

Acetabularia, Arabidopsis, Brassica, Capsicum, Chlamydomonas, Cucurbita, Dunaliella, Euglena, Flaveria, Glycine, Helianthus, Hordeum, Lemna, Lolium, Lycopersion, Malus, Medicago, Mesembryanthemum, Nicotiana, Oenotherea, Oryza, Petunia, Phaseolus, Physcomitrella, Pinus, Pisum, Raphanus, Silene, Sinapis, Solanum, Spinacea, Stevia, Synechococcus, Triticum and Zea.

Advantageously plastidial transit peptides, which are beneficially used in an embodiment of the process of the present invention, are derived from the nucleic acid sequence encoding a protein selected from the group consisting of

ribulose bisphosphate carboxylase/oxygenase, 5-enolpyruvyl-shikimate-3-phosphate synthase, acetolactate synthase, chloroplast ribosomal protein CS17, Cs protein, ferredoxin, plastocyanin, ribulose bisphosphate carboxylase activase, tryptophan synthase, acyl carrier protein, plastid chaperonin-60, cytochrome c552, 22-kDA heat shock protein, 33-kDa Oxygen-evolving enhancer protein 1, ATP synthase y subunit, ATP synthase 6 subunit, chlorophyll-a/b-binding proteinII-1, Oxygen-evolving enhancer protein 2, Oxygen-evolving enhancer protein 3, photosystem I: P21, photosystem I: P28, photosystem I: P30, photosystem I: P35, photosystem I: P37, glycerol-3-phosphate acyltransferases, chlorophyll a/b binding protein, CAB2 protein, hydroxymethyl-bilane synthase, pyruvate-orthophosphate dikinase, CAB3 protein, plastid ferritin, ferritin, early light-inducible protein, glutamate-1-semialdehyde aminotransferase, protochlorophyllide reductase, starch-granule-bound amylase synthase, light-harvesting chlorophyll a/b-binding protein of photosystem II, major pollen allergen Lol p 5a, plastid CIpB ATP-dependent protease, superoxide dismutase, ferredoxin NADP oxidoreductase, 28-kDa ribonucleoprotein, 31-kDa ribonucleoprotein, 33-kDa ribonucleoprotein, acetolactate synthase, ATP synthase CF0 subunit 1, ATP synthase CFO subunit 2, ATP synthase CF0 subunit 3, ATP synthase CF0 subunit 4, cytochrome f, ADP-glucose pyrophosphorylase, glutamine synthase, glutamine synthase 2, carbonic anhydrase, GapA protein, heat-shock-protein hsp21, phosphate translocator, plastid CIpA ATP-dependent protease, plastid ribosomal protein CL24, plastid ribosomal protein CL9, plastid ribosomal protein PsCL18, plastid ribosomal protein PsCL25, DAHP synthase, starch phosphorylase, root acyl carrier protein II, betaine-aldehyde dehydrogenase, GapB protein, glutamine synthetase 2, phosphoribulokinase, nitrite reductase, ribosomal protein L12, ribosomal protein L13, ribosomal protein L21, ribosomal protein L35, ribosomal protein L40, triose phosphate-3-phosphoglyerate-phosphate translocator, ferredoxin-dependent glutamate synthase, glyceraldehyde-3-phosphate dehydrogenase, NADP-dependent malic enzyme and NADP-malate dehydrogenase.

More preferred the nucleic acid sequence encoding a plastidal transit peptide is derived from a nucleic acid sequence encoding a protein finally resided in the plastid and stemming from an organism selected from the group consisting of the species: Acetabularia mediterranea, Arabidopsis thaliana, Brassica campestris, Brassica napus, Capsicum annuum, Chlamydomonas reinhardtii, Cucurbita moschata, Dunaliella salina, Dunaliella tertiolecta, Euglena gracilis, Flaveria trinervia, Glycine max, Helianthus annuus, Hordeum vulgare, Lemna gibba, Lolium perenne, Lycopersion esculentum, Malus domestica, Medicago falcata, Medicago sativa, Mesembryanthemum crystallinum, Nicotiana plumbaginifolia, Nicotiana sylvestris, Nicotiana tabacum, Oenotherea hookeri, Oryza sativa, Petunia hybrida, Phaseolus vulgaris, Physcomitrella patens, Pinus tunbergii, Pisum sativum, Raphanus sativus, Silene pratensis, Sinapis alba, Solanum tuberosum, Spinacea oleracea, Stevia rebaudiana, Synechococcus, Synechocystis, Triticum aestivum and Zea mays.

Even more preferred nucleic acid sequences are encoding plastidal transit peptides as disclosed by von Heijne et al. (Plant Molecular Biology Reporter, 9 (2), 104 (1991)), which are hereby incorporated by reference. Table a shows some examples of the transit peptide sequences disclosed by von Heijne et al. According to the disclosure of the invention especially in the examples the skilled worker is able to link other nucleic acid sequences disclosed by von Heijne et al. to the respective nucleic acid sequences shown in Table I, columns 5 or 8, preferably the respective coding region thereof, or homologs or fragments thereof. Most preferred nucleic acid sequences encoding transit peptides are derived from the genus Spinacia such as chloroplast 30S ribosomal protein PSrp-1, root acyl carrier protein II, acyl carrier protein, ATP synthase: γ subunit, ATP synthase: σ subunit, cytochrom f, ferredoxin I, ferredoxin NADP oxidoreductase (=FNR), nitrite reductase, phosphoribulokinase, plastocyanin or carbonic anhydrase. The skilled worker will recognize that various other nucleic acid sequences encoding transit peptides can easily isolated from plastid-localized proteins, which are expressed from nuclear genes as precursors and are then targeted to plastids. Such transit peptides encoding sequences can be used for the construction of other expression constructs. The transit peptides advantageously used in the inventive process and which are part of the inventive nucleic acid sequences and proteins are typically 20 to 120 amino acids, preferably 25 to 110, 30 to 100 or 35 to 90 amino acids, more preferably 40 to 85 amino acids and most preferably 45 to 80 amino acids in length and function post-translationally to direct the protein to the plastid, preferably to the chloroplast. The nucleic acid sequences encoding such transit peptides are localized upstream of nucleic acid sequence encoding the mature protein. For the correct molecular joining of the transit peptide encoding nucleic acid and the nucleic acid encoding the protein to be targeted it is sometimes necessary to introduce additional base pairs at the joining position, which form restriction enzyme recognition sequences useful for the molecular joining of the different nucleic acid molecules. This procedure might lead to very few additional amino acids at the N-terminal of the mature imported protein, which usually and preferably do not interfere with the protein function. In any case, the additional base pairs at the joining position which form restriction enzyme recognition sequences have to be chosen with care, in order to avoid the formation of stop codons or codons which encode amino acids with a strong influence on protein folding, like e.g. proline. It is preferred that such additional codons encode small n.d. structural flexible amino acids such as glycine or alanine.

As mentioned above the nucleic acid sequences coding for the proteins as shown in Table II, application no. 1, column 5 or 8, or homologs or fragments thereof, are joined to a nucleic acid sequence encoding a plastidic transit peptide, e.g, if for the nucleic acid molecule in Table I, column 6 the term “plastidic”is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in Table II, application no. 1, column 5 or 8, or homologs or fragments thereof, e.g, if for the nucleic acid molecule in Table I, column 6 the term “plastidic” is indicated.

Other plastid transit peptides are disclosed by Schmidt et al. (J. Biol. Chem. 268 (36), 27447 (1993)), Della-Cioppa et al. (Plant. Physiol. 84, 965 (1987)), de Castro Silva Filho et al. (Plant Mol. Biol. 30, 769 (1996)), Zhao et al. (J. Biol. Chem. 270 (11), 6081 (1995)), Römer et al. (Biochem. Biophys. Res. Commun., 196 (3), 1414 (1993)), Keegstra et al. (Annu. Rev. Plant Physiol. Plant Mol. Biol. 40, 471 (1989)), Lubben et al. (Photosynthesis Res. 17, 173 (1988)) and Lawrence et al. (J. Biol. Chem. 272, (33), 20357 (1997)), Cho et al. (Planta 224, 598 (2006)), Lucia et al. (Transgenic R. 17 (4), 529 (2008)), Murayama et al. (Planta 225 (5), 1193 (2007)). A general review about targeting is disclosed by Allison R. Kermode in Critical Reviews in Plant Science 15 (4), 285 (1996).

Favored plastidal transit peptide sequences, which are used in an embodiment of the inventive process and which form part of the inventive nucleic acid sequences are generally enriched in hydroxylated amino acid residues (serine and threonine), with these two residues generally constituting 20-35% of the total. They often have an amino-terminal region empty of Gly, Pro, and charged residues. Furthermore they have a number of small hydrophobic amino acids such as valine and alanine and generally acidic amino acids are lacking. In addition they generally have a middle region rich in Ser, Thr, Lys and Arg. Overall they have very often a net positive charge.

Advantageously mitochondrial transit peptides, which are beneficially used in an embodiment of the process of the present invention, are derived from the nucleic acid sequence encoding a protein selected from the group consisting of 22 kDA heat shock protein; 70 kDA heat shock protein; 83 kDA heat shock protein; 40S ribosomal protein S19; 50S ribosomal protein L15; ribosomal protein L29; 22 kDA PSST protein of complex I; 2-oxoacid dehydrogenase family protein; 2-oxoglutarate/malate translocator; 3-methyl-2-oxobutanoate hydroxymethyltransferase; 3-Methylcrotonyl-coenzyme A carboxylase (MCCase); 7,8 - Dihydropteroate synthase (DHPS)/6-hydroxymethyl-7,8-dihydropterine pyrophosphokinase (HPPK); aconitate hydratase; acyl carrier protein (ACP); ADP/ATP translocase; alanyl-tRNA synthetase; alcohol dehydrogenase (ADH); alternative oxidase (AOX); aminoacylt-RNA ligase; asparate aminotransferase; ATP synthase alpha subunit; ATP synthase beta subunit; ATP synthase delta subunit; ATP synthase epsilon subunit; ATP synthase gamma subunit; ATP-dependent Clp protease-proteolytic subunit; Chaperonin 60- CPN60; Chaperonin 60 (2)-CPN60-2; Chaperonin 60(1)-CPN60-1; citrate synthase; cytochrome b-c1complex subunit Rieske FeS Protein; cytochrome c reductase-processing peptidase subunit II; dihydrolipoamide S-acetyltransferase; farnesyl-diphosphate synthase 1; formate dehydrogenase; fumarate hydratase; gamma carbonic anhydrase protein (gammaCA); gamma carbonic anhydrase-like protein 1 (gammaCAL1); gamma carbonic anhydrase-like protein 2 (gammaCAL2); gamma-aminobutyric acid transaminase (GABA-T); glutathione reductase (GR); glycine decarboxylase subunit H; glycine decarboxylase subunit L; glycine decarboxylase subunit P; glycine decarboxylase subunit T; isovaleryl-CoA dehydrogenase (IVD); lipoamide dehydrogenase; malate oxidoreductase; manganese superoxide dismutase (Mn)SOD; methylmalonate-semialdehyde dehydrogenase; mitochondrial-processing peptidase beta subunit (MPP); mitochondrial-processing peptidase subunit alpha (MPP); monodehydroascorbate reductase (MDHAR; NAD dependent isocitrate dehydrogenase; NAD dependent malate dehydrogenase; NAD-dependent malic enzyme; NAD-dependent malic enzyme 59 kDa isoform; NAD-dependent malic enzyme 62 kDa isoform; NAD-dependent malic enzyme 65 kDa isoform; NADH ubiquinone oxidoreductase 29 kDa subunit; NADH-ubiquinone oxidoreductase 18 kDa subunit; NADH-ubiquinone oxidoreductase 20 kDa subunit; NADH-ubiquinone oxidoreductase 23 kDa subunit; NAD-Hubiquinone oxidoreductase 75 kDa subunit; NADP dependent isocitrate dehydrogenase; NADP dependent malate dehydrogenase; nucleoside diphosphate kinase; nucleoside diphosphate kinase III; o-acetylserine (thiol) lyase (OAS-TL) ; propionyl-CoA carboxylase; protoporphyrinogen IX oxidase; pyruvate dehydrogenase E1 component subunit alpha; serine acetyltransferase (SAT); serine hydroxymethyltransferase; succinate dehydrogenase (SDH); succinic semialdehyde dehydrogenase (SSADH); succinyl-CoA ligase (GDP-forming) alpha-chain; succinyl-CoA ligase [GDP-forming] subunit beta; thiosulfate sulfurtransferase; threonyl-tRNA synthetase; trans-2-enoyl-CoA reductase; translocase inner membrane (TIM); translocase outer membrane (TOM); tRNA synthetase class I and ubiquinol cytochrome C oxidoreductase complex.

More preferred the nucleic acid sequence encoding a mitochondrial transit peptide is derived from a nucleic acid sequence encoding a protein finally resided in the mitochondrion and stemming from an organism selected from the group consisting of the species: Acetabularia mediterranea, Arabidopsis thaliana, Brassica campestris, Brassica napus, Capsicum annuum, Chlamydomonas reinhardtii, CururbitaCucurbita moschata, Daucus carota, Dunaliella salina, Dunaliella tertiolecta, Euglena gracilis, Flaveria trinervia, Glycine max, Helianthus annuus, Hordeum vulgare, Lactuca sativa, Lemna gibba, Lolium perenne, Lycopersion esculentum, Malus domestica, Medicago falcata, Medicago sativa, Mesembryanthemum crystallinum, Nicotiana plumbaginifolia, Nicotiana sylvestris, Nicotiana tabacum, Oenotherea hookeri, Oryza sativa, Petunia hybrida, Phaseolus vulgaris, Physcomitrella patens, Pinus tunbergii, Pisum sativum, Pyrus pyrifolia, Raphanus sativus, Saccharum officinarum, Silene pratensis, Sinapis alba, Solanum tuberosum, Spinacea oleracea, Stevia rebaudiana, Synechococcus, Synechocystis, Triticum aestivum and Zea mays.

Even more preferred nucleic acid sequences are encoding mitochondrial transit peptides as disclosed by White and Scandalios (Proc. Natl. Acad. Sci., 86 (10), 3534 (1989)), Cho et al (Plant Physiol. 149, 745 (2009)), Chatre et al (Journal of Experimental Botany, 60 (3), 741 (2009)), Murayama et al (Planta 225 (5), 1193 (2007) and Manzano et al (2006) , which are hereby incorporated by reference. Table b shows some examples of the transit peptide sequences disclosed by White and Scandalios (1989), Cho et al (2009), Chatre et al (2009), Murayama et al (2007) and Manzano et al (2006), Glaser et al (Plant Mol Biol. 38 (1-2), 311 (1998) and Huang et al (Plant Phys. 150, 1272 (2009)). According to the disclosure of the invention especially in the examples the skilled worker is able to link other nucleic acid sequences disclosed by Glaser et al (Plant Mol Biol. 38 (1-2), 311 (1998)) and Huang et al (Plant Phys. 150, 1272 (2009)) to the respective nucleic acid sequences shown in Table I, columns 5 or 8, preferably the coding region thereof, or a homolog or a fragment thereof. Most preferred nucleic acid sequences encoding transit peptides are derived from the genus Arabidopsis thaliana such as mitochondrial malate oxidoreductase, manganese superoxide dismutase (Mn)SOD, translocase inner membrane (TIM), translocase outer membrane (TOM), ATP synthase alpha subunit, ATP synthase beta subunit, ATP synthase gamma subunit, ATP synthase delta subunit, ATP synthase epsilon subunit, ADP/ATP translocase, aconitate hydratase and isovaleryl-CoA dehydrogenase (IVD). The skilled worker will recognize that various other nucleic acid sequences encoding transit peptides can easily isolated from mitochondrion-localized proteins, which are expressed from nuclear genes as precursors and are then targeted to mitochondria. Such transit peptides encoding sequences can be used for the construction of other expression constructs. The transit peptides advantageously used in the inventive process and which are part of the inventive nucleic acid sequences and proteins are typically 20 to 120 amino acids, preferably 25 to 110, 30 to 100 or 35 to 90 amino acids, more preferably 40 to 85 amino acids and most preferably 45 to 80 amino acids in length and function post-translationally to direct the protein to the mitochondrion. The nucleic acid sequences encoding such transit peptides are localized upstream of nucleic acid sequence encoding the mature protein. For the correct molecular joining of the transit peptide encoding nucleic acid and the nucleic acid encoding the protein to be targeted it is sometimes necessary to introduce additional base pairs at the joining position, which forms restriction enzyme recognition sequences useful for the molecular joining of the different nucleic acid molecules. This procedure might lead to very few additional amino acids at the N-terminal of the mature imported protein, which usually and preferably do not interfere with the protein function. In any case, the additional base pairs at the joining position which forms restriction enzyme recognition sequences have to be chosen with care, in order to avoid the formation of stop codons or codons which encode amino acids with a strong influence on protein folding, like e.g. proline. It is preferred that such additional codons encode small n.d. structural flexible amino acids such as glycine or alanine.

As mentioned above the nucleic acid sequences coding for the proteins as shown in the respective line in Table II, application no. 1, column 5 or 8, or homologs or fragments thereof, are joined to a nucleic acid sequence encoding a mitochondric transit peptide, e.g., if for the respective nucleic acid molecule in Table I, column 6 the term “mitochondrial” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in the respective line in Table II, application no. 1, column 5 or 8, or homologs or fragments thereof, e.g, if for the nucleic acid molecule in the respective line in Table I, column 6 the term “mitochondrial” is indicated. (For the avoidance of doubt, the term “mitochondric” and “mitochondrial” are interchangeable.)

Other mitochondric transit peptides are disclosed by Cho et al. (Plant Physiol. 149, 745 (2009)), Gnanasambandam et al. (Functional Plant Biology 35(2), 166, (2008)), Subbaiah et al. (J. Biol.Chem. 281 (23), 15625 (2006)), Murcha et al. (Plant Physiol. 138, 2134 (2005)), Chatre et al. (Journal of Experimental Botany, 60 (3), 741 (2009)), Millar and Heazlewood (Plant Physiol. 131, 443 (2003)), White and Scandalios (Proc. Natl. Acad. Sci., 86 (10), 3534 (1989)), Laloi et al. (Proc. Natl. Acad. Sci., 98 (24), 14144 (2001)), Barranco-Medina et al. (Plant Physiol. and Biochem. 45, 729 (2007)), Olejnik et al. (FEBS J. 274 (18), 4877 (2007)), Fabre et al. (Plant Cell and Env. 30 (5), 617 (2007)), Murayama et al. (Planta 225 (5), 1193 (2007)), Bathgate et al. (Eur. J. Biochem. 183, 303 (1989)), Winning et al. (Plant J. 2, 763 (1992)), Murcha et al. (Plant Physiol. 143, 199 (2007)), Lehnerer et al. (Botanica Acta, 107, 306 (1994)), Kimura et al. (J. Biol.Chem. 265 (11), 6079 (1990)), Yang et al. (Plant Mol. Biol., 62, 951 (2006)).

Favored mitochondric transit peptide sequences, which are used in an embodiment of the inventive process and which form part of the inventive nucleic acid sequences are generally enriched in hydroxylated amino acid residues (serine and threonine), with these two residues generally constituting 20-35% of the total. They often have an amino-terminal region empty of Gly, Pro, and charged residues. Furthermore they have a number of small hydrophobic amino acids such as valine and alanine and generally acidic amino acids are lacking. In addition they generally have a middle region rich in Ser, Thr, Lys and Arg. Overall they have very often a net positive charge.

Nucleic acid sequences coding for the transit peptides may be chemically synthesized either in part or wholly according to structure of transit peptide sequences disclosed in the prior art. Said natural or chemically synthesized sequences can be directly linked to the sequences encoding the mature protein or via a linker nucleic acid sequence, which may be typically less than 500 base pairs, preferably less than 450, 400, 350, 300, 250 or 200 base pairs, more preferably less than 150, 100, 90, 80, 70, 60, 50, 40 or 30 base pairs and most preferably less than 25, 20, 15, 12, 9, 6 or 3 base pairs in length and are in frame to the coding sequence. Furthermore favorable nucleic acid sequences encoding transit peptides may comprise sequences derived from more than one biological and/or chemical source and may include a nucleic acid sequence derived from the amino-terminal region of the mature protein, which in its native state is linked to the transit peptide. In a preferred embodiment of the invention said amino-terminal region of the mature protein is typically less than 150 amino acids, preferably less than 140, 130, 120, 110, 100 or 90 amino acids, more preferably less than 80, 70, 60, 50, 40, 35, 30, 25 or 20 amino acids and most preferably less than 19, 18, 17, 16, 15, 14, 13, 12, 11 or 10 amino acids in length. But even shorter or longer stretches are also possible. In addition target sequences, which facilitate the transport of proteins to other cell compartments such as the vacuole, endoplasmic reticulum, golgi complex, glyoxysomes, peroxisomes, mitochondria (in case of a plastidal transit peptide) or plastids, especially chloroplasts (in case of a mitochondric transit peptide) may be also part of the inventive nucleic acid sequence. The proteins translated from said inventive nucleic acid sequences are a kind of fusion proteins that means the nucleic acid sequences encoding the plastidal transit peptide for example the ones shown in Table a, preferably the last one of the Table a are joint to the respective nucleic acid sequences shown in Table I, application no. 1, columns 5 or 8, especially the coding region thereof, or homologs or fragments thereof, in case of nucleic acid sequences being expressed in plastids, and that means the nucleic acid sequences encoding the mitochondric transit peptide for example the ones shown in Table b, preferably the last one of the Table b are joint to the respective nucleic acid sequences shown in Table I, application no. 1, columns 5 or 8, especially the coding region thereof, or homologs or fragments thereof, in case of nucleic acid sequences being expressed in mitochondria. The person skilled in the art is able to join said sequences in a functional manner. Advantageously the transit peptide part is cleaved off from the protein part shown in Table II, application no. 1, columns 5 or 8, or a homolog or fragment thereof, during or after the transport into the organelles, preferably into the plastids or mitochondria, respectively.

All products of the cleavage of the preferred transit peptide shown in the last line of Table a have preferably the N-terminal amino acid sequences QIA CSS or QIA EFQLTT in front of the start methionine of the respective protein metioned in Table II, application no. 1, column 5 or 8. or homologs or fragments thereof. Other short amino acid sequences of an range of 1 to 20 amino acids preferable 2 to 15 amino acids, more preferable 3 to 10 amino acids most preferably 4 to 8 amino acids are also possible in front of the start methionine of the protein metioned in Table II, application no. 1, columns 5 or 8, or homologs or fragments thereof. In case of the amino acid sequence QIA CSS the three amino acids in front of the start methionine are stemming from the LIC (=ligatation independent cloning) cassette. Said short amino acid sequence is preferred in the case of the expression of E. coli genes. In case of the amino acid sequence QIA EFQLTT the six amino acids in front of the start methionine are stemming from the LIC cassette. Said short amino acid sequence is preferred in the case of the expression of S. cerevisiae genes. The skilled worker knowns that other short sequences are also useful in the expression of the respective nucleic acid molecule comprising a polynucleotide as depicted in Table I, application no. 1, column 5 or 8, preferably the coding region thereof, or a homolog or a fragment thereof. Furthermore the skilled worker is aware of the fact that there is not a need for such short sequences in the expression of the genes.

TABLE a
Examples of plastidal transit peptides disclosed by von Heijne et al.

| Trans | | | SEQ | |
|---|---|---|---|---|
| Pep | Organism | Transit Peptide | ID NO. | Reference |
| 1 | Acetabularia | MASIMMNKSVVLSKECAKPLATPKVTLN | 1 | Mol. Gen. |
| | mediterranea | KRGFATTIATKNREMMVWQPFNNKMFET | | Genet. 218, |
| | | FSFLPP | | 445 (1989) |
| 2 | Arabidopsis | MAASLQSTATFLQSAKIATAPSRGSSHL | 2 | EMBO J. 8, |
| | thaliana | RSTQAVGKSFGLETSSARLTCSFQSDFK | | 3187 (1989) |
| | | DFTGKCSDAVKIAGFALATSALVVSGAS | | |
| | | AEGAPK | | |
| 3 | Arabidopsis | MAQVSRICNGVQNPSLICNLSKSSQRKS | 3 | Mol. Gen. |
| | thaliana | PLSVSLKTQQHPRAYPISSSWGLKKSGM | | Genet. 210, |
| | | TLIGSELRPLKVMSSVSTAEKASEIVLQ | | 437 (1987) |
| | | PIREISGLIKLP | | |
| 4 | Arabidopsis | MAAATTTTTTSSSISFSTKPSPSSSKSP | 4 | Plant |
| | thaliana | LPISRFSLPFSLNPNKSSSSSRRRGIKS | | Physiol. 85, |
| | | SSPSSISAVLNTTTNVTTTPSPTKPTKP | | 1110 (1987) |
| | | ETFISRFAPDQPRKGA | | |
| 5 | Arabidopsis | MITSSLTCSLQALKLSSPFAHGSTPLSS | 5 | J. Biol. |
| | thaliana | LSKPNSFPNHRMPALVPV | | Chem. 265, |
| | | | | 2763 (1990) |
| 6 | Arabidopsis | MASLLGTSSSAIWASPSLSSPSSKPSSS | 6 | EMBO J. |
| | thaliana | PICFRPGKLFGSKLNAGIQIRPKKNRSR | | 9, 1337 |
| | | YHVSVMNVATEINSTEQVVGKFDSKKSA | | (1990) |
| | | RPVYPFAAI | | |
| 7 | Arabidopsis | MASTALSSAIVGTSFIRRSPAPISLRSLP | 7 | Plant |
| | thaliana | SANTQSLFGLKSGTARGGRVVAM | | Physiol. 93, |
| | | | | 572 (1990) |
| 8 | Arabidopsis | MAASTMALSSPAFAGKAVNLSPAASEVL | 8 | Nucl. Acids |
| | thaliana | GSGRVTNRKTV | | Res. 14, |
| | | | | 4051 (1986) |
| 9 | Arabidopsis | MAAITSATVTIPSFTGLKLAVSSKPKTL | 9 | Gene 65, |
| | thaliana | STISRSSSATRAPPKLALKSSLKDFGVI | | 59 (1988) |
| | | AVATAASIVLAGNAMAMEVLLGSDDGSL | | |
| | | AFVPSEFT | | |
| 10 | Arabidopsis | MAAAVSTVGAINRAPLSLNGSGSGAVSA | 10 | Nucl. Acids |
| | thaliana | PASTFLGKKWTVSRFAQSNKKSNGSFKV | | Res. 17, |
| | | LAVKEDKQTDGDRWRGLAYDTSDDQIDI | | 2871 (1989) |
| 11 | Arabidopsis | MkSSMLSSTAWTSPAQATMVAPFTGLKS | 11 | Plant Mol. |
| | thaliana | SASFPVTRKANNDITSITSNGGRVSC | | Biol. 11, |
| | | | | 745 (1988) |
| 12 | Arabidopsis | MAASGTSATFRASVSSAPSSSSQLTHLK | 12 | Proc. Natl. |
| | thaliana | SPFKAVKY TPLPS SRSKSSSFSVSCT | | Acad. Sci. |
| | | IAKDPPVLMAAGSDPALWQRPDSFGRFG | | USA 86, |
| | | KFGGKYVPE | | 4604 (1989) |
| 13 | Brassica | MSTTFCSSVCMQATSLAATTRISFQKPA | 13 | Nucl. Acids |
| | campestris | LVSTTNLSFNLRRSIPTRFSISCAAKPE | | Res. 15, |
| | | TVEKVSKIVKKQLSLKDDQKVVAE | | 7197 (1987) |
| 14 | Brassica | MATTFSASVSMQATSLATTTRISFQKPV | 14 | Eur. J. Bio- |
| | napus | LVSNHGRTNLSFNLSRTRLSISC | | chem. 174, |
| | | | | 287 (1988) |
| 15 | Chlamydomonas | MQALSSRVNIAAKPQRAQRLWRAEEVKA | 15 | Plant Mol. |
| | reinhardtii | APKKEVGPKRGSLVK | | Biol. 12, |
| | | | | 463 (1989) |
| 16 | Cucurbita | MAELIQDKESAQSAATAAAASSGYERRN | 16 | FEBS Lett. |
| | moschata | EPAHSRKFLEVRSEEELLSCIKK | | 238, 424 |
| | | | | (1988) |
| 17 | Spinacea | MSTINGCLTSISPSRTQLKNTSTLRPTF | 17 | J. Biol. |
| | oleracea | IANSRVNPSSSVPPSLIRNQPVFAAPAP | | Chem. 265, |
| | | IITPTL | | 5414 (1990) |
| 18 | Spinacea | MTTAVTAAVSFPSTKTTSLSARCSSVIS | 18 | Curr. |
| | oleracea | PDKISYKKVPLYYRNVSATGKMGPIRAQ | | Genet. 13, |
| | | IASDVEAPPPAPAKVEKMS | | 517 (1988) |
| 19 | Spinacea | MTTAVTAAVSFPSTKTTSLSARSSSVIS | 19 | |
| | oleracea | PDKISYKKVPLYYRNVSATGKMGPIRA | | |

TABLE b
Examples of mitochondric transit peptides disclosed by
White and Scandalios (1989); Cho et al (2009); Chatre et al
(2008); Murayama et al (2007); Manzano et al (2006):

| Trans | | | SEQ | |
|---|---|---|---|---|
| Pep | Organism | Transit Peptide | ID NO. | Reference |
| 20 | Zea mays | MALRTLASKKVLSFPFGGAGRPLAAA | 63 | Proc. Nat. |
| | | ASARGV | | Acad. Sci. |
| | | | | 86, 3534 |
| | | | | (1989) |
| 21 | Oryza sativa | MGKAAAVGTAVVVAAAVGVAVVLA | 64 | Plant |
| | | | | Physiol |
| | | | | 149, 745 |
| | | | | (2009) |
| 22 | Oryza sativa | MGKGTVVGTAVVVCAAAAAAVGVAVV | 65 | Plant |
| | | VS | | Physiol |
| | | | | 149, 745 |
| | | | | (2009) |
| 23 | Arabidosis | MIITTHKRDINLLVLQLGAALAVSFA | 66 | J. Exp. Bot. |
| | thaliana | GFLFARFRKNTKR | | 60 (3), 741 |
| | | | | (2009) |
| 24 | Oryza sativa | MAAAAISHLRRGAPRHARALYLSTR- | 67 | Planta 225 |
| | | RFSSSSAAGVAPLAAVAASARR- | | (5), 1193 |
| | | LLSTSVDSGASSGESYKPPLFDPF- | | (2007) |
| | | RAASLASSAPPLESPPIEELPDDA- | | |
| | | TPPPEEEPGLPAPEKDPVATACQHE- | | |
| | | LEGLKAVETVRSRKESTEEKEAWSL- | | |
| | | LGRSVVSYCGTA | | |
| 25 | Arabidosis | MSVSCCCRNLGKTIKKAIPSHHLHL- | 68 | Plant Mol. |
| | thaliana | RSLGGSLYRRRIQSSSMETDLKS | | Bio. 61, |
| | | | | 195 (2006) |

Alternatively to the targeting of the respective sequences shown in Table II, application no. 1, columns 5 or 8, or homologs or fragments thereof, preferably of sequences in general encoded in the nucleus with the aid of the targeting sequences mentioned for example in Table a alone, Table b alone, or in combination with other targeting sequences preferably into plastids or mitochondria, respectively, the nucleic acids of the invention can directly be introduced into the plastidal or mitochondrial genome. Therefore in a preferred embodiment the respective nucleic acid sequences shown in the respective line in Table I, application no. 1, columns 5 or 8, preferably the coding region thereof, or homologs or fragments thereof, are directly introduced and expressed in plastids e.g, if for the nucleic acid molecule in the respective line in Table I, column 6, the term “plastidic” is indicated, or are directly introduced and expressed in mitochondria e.g., if for the nucleic acid molecule in the respective line in Table I, column 6 the term “mitochondrial” is indicated.

The term “introduced” in the context of this specification shall mean the insertion of a nucleic acid sequence into the organism by means of a “transfection”, “transduction” or preferably by “transformation”.

A plastid, such as a chloroplast, or a mitochondrion, has been “transformed” by an exogenous (preferably foreign) nucleic acid sequence if a nucleic acid sequence has been introduced into the plastid or mitochondrion that means that this sequence has crossed the membrane or the membranes of the plastid or of the mitochondrion. The exogenous, preferably foreign, DNA may be integrated (covalently linked) into plastid or mitochondrial DNA making up the genome of the plastid or mitochondrion, or it may remain unintegrated (e.g., by including a chloroplast origin of replication). “Stably” integrated DNA sequences are those, which are inherited through plastid or mitochondrion replication, thereby transferring new plastids or mitochondria, with the features of the integrated DNA sequence to the progeny.

For expression a person skilled in the art is familiar with different methods to introduce the nucleic acid sequences into different organelles, such as the preferred plastids or mitochondria. Such methods are for example disclosed by Pal Maiga (Annu. Rev. Plant Biol. 55, 289, (2004)), Evans T. (WO 2004/040973), McBride K. E. et al. (U.S. Pat. No. 5,455,818), Daniell H. et al. (U.S. Pat. No. 5,932,479 and U.S. Pat. No. 5,693,507) and Straub J. M. et al. (U.S. Pat. No. 6,781,033). A preferred method is the transformation of microspore-derived hypocotyl or cotyledonary tissue (which are green and thus contain numerous plastids) leaf tissue and afterwards the regeneration of shoots from said transformed plant material on selective medium. As methods for the transformation bombarding of the plant material or the use of independently replicating shuttle vectors are well known by the skilled worker. But also a PEG-mediated transformation of plastids or Agrobacterium transformation with binary vectors are possible. Useful markers for the transformation of plastids are positive selection markers for example the chloramphenicol-, streptomycin-, kanamycin-, neomycin-, amikamycin-, spectinomycin-, triazine- and/or lincomycin-resistance genes. As additional markers named in the literature often as secondary markers, genes coding for the resistance against herbicides such as phosphinothricin (=glufosinate, BASTA™, Liberty™, encoded by the bar gene), glyphosate (=N-(phosphonomethyl)glycine, Roundup Ready™, encoded by the 5-enolpyruvylshikimaete-3-phosphate synthase gene=epsps), sulfonylurea (e.g. =Staple™, encoded by the acetolactate synthase gene), imidazolinone (=IMI, e.g. imazethapyr, imazamox, Clearfield™, encoded by the acetohydroxyacid synthase (AHAS) gene, also known as acetolactate synthase (ALS) gene) or bromoxynil (=Buctril™, encoded by the oxy gene) or genes coding for antibiotics such as hygromycin or G418 are useful for further selection. Such secondary markers are useful in the case when most genome copies are transformed. In addition negative selection markers such as the bacterial cytosine deaminase (encoded by the codA gene) are also useful for the transformation of organelles, especially plastids.

Thus, in one embodiment, an activity disclosed herein as being conferred by a polypeptide shown in the respective line in Table II, or a homolog or a fragment thereof, is increased or generated by linking the polypeptide disclosed in respective line in Table II, or a homolog or a fragment thereof, or a polypeptide conferring the same said activity with a targeting signal as herein described, if in the respective line in column 6 of Table II the term “plastidic” is listed for said polypeptide. For example, the polypeptide described can be linked to the targeting signal shown in Table a.

Accordingly, in the method of the invention for producing a transgenic plant with increased fine chemical production as compared to a corresponding, e.g. non-transformed, wild type plant, comprising transforming a non-human organism, preferably a plant cell, a plant or a part thereof, with the mentioned nucleic acid molecule, said nucleic acid molecule selected from said mentioned group encodes for a polypeptide conferring said activity being linked to a targeting signal as mentioned herein, e.g. as mentioned in Table a, e.g. if in the respective line in column 6 of Table II the term “plastidic” is listed for the encoded polypeptide.

To increase the possibility of identification of transformants it is possible to use reporter genes other then the aforementioned resistance genes or in addition to said genes. Reporter genes are for example β-galactosidase-, β-glucuronidase-(GUS), alkaline phosphatase- and/or green-fluorescent protein-genes (GFP).

For the process of the present invention it may be of great advantage that by transforming the plastids the intraspecies specific transgene flow is blocked, because a lot of species such as corn, cotton and rice have a strict maternal inheritance of plastids. By placing the respective genes specified in the respective line in Table I, application no. 1, columns 5 or 8, preferably the coding region, or homologs or fragments thereof, e.g. if for the nucleic acid molecule in the respective line in column 6 of Table I the term “plastidic” is indicated, in the plastids of plants, these genes will not be present in the pollen of said plants.

A further preferred embodiment of the invention relates to the use of so called “chloroplast localization sequences”, in which a first RNA sequence or molecule is capable of transporting or “chaperoning” a second RNA sequence, such as a RNA sequence transcribed from the sequences depicted in the respective line in Table I, application no. 1, columns 5 or 8, preferably the coding region theeof, or a homolog or a fragment thereof, or a sequence encoding a protein, as depicted in the respective line in Table II, application no. 1, columns 5 and 8, or a homolog or a fragment thereof, from an external environment inside a cell or outside a plastid into a chloroplast. In one embodiment the chloroplast localization signal is substantially similar or complementary to a complete or intact viroid sequence. The chloroplast localization signal may be encoded by a DNA sequence, which is transcribed into the chloroplast localization RNA. The term “viroid” refers to a naturally occurring single stranded RNA molecule (Flores, C. R. Acad. Sci. III. 324 (10), 943 (2001)). Viroids usually contain about 200-500 nucleotides and generally exist as circular molecules. Examples of viroids that contain chloroplast localization signals include but are not limeted to ASBVd, PLMVd, CChMVd and ELVd. The viroid sequence or a functional part of it can be fused to the sequences depicted in the respective line in Table I, application no. 1, columns 5 or 8, preferably the coding region thereof, or a homolog or a fragment thereof, or a sequence encoding a protein, as depicted in the respective line in Table II, application no. 1, columns 5 or 8, or a homolog or a fragment thereof, in such a manner that the viroid sequence transports a sequence transcribed from a sequence as depicted in the respective line in Table I, application no. 1 columns 5 or 8, preferably the coding region thereof, or a homolog or a fragment thereof, or a sequence encoding a protein as depicted in th erespective line in Table II, application no. 1 columns 5 or 8, or a homolog or a fragment thereof, into the chloroplasts, e.g. if for the nucleic acid molecule in column 6 of Table I the term “plastidic” is indicated. A preferred embodiment uses a modified ASBVd (Navarro et al., Virology. 268 (1), 218 (2000)).

In a further specific embodiment the protein to be expressed in the plastids such as the proteins depicted in the respective line in Table II, application no.1, columns 5 or 8, or a homolog or a fragment thereof, e.g. if for the nucleic acid molecule in the respective line in column 6 of Table I the term “plastidic” is indicated, are encoded by different nucleic acids. Such a method is disclosed in WO 2004/040973, which shall be incorporated by reference. WO 2004/040973 teaches a method, which relates to the translocation of an RNA corresponding to a gene or gene fragment into the chloroplast by means of a chloroplast localization sequence. The genes, which should be expressed in the plant or plant cells, are split into nucleic acid fragments, which are introduced into different compartments in the plant e.g. the nucleus, the plastids and/or mitochondria. Additionally plant cells are described in which the chloroplast contains a ribozyme fused at one end to an RNA encoding a fragment of a protein used in the inventive process such that the ribozyme can trans-splice the translocated fusion RNA to the RNA encoding the gene fragment to form and as the case may be reunite the nucleic acid fragments to an intact mRNA encoding a functional protein for example as disclosed in the respective line in Table II, application no. 1, columns 5 or 8, or a homolog or a fragment thereof.

In a preferred embodiment of the invention the nucleic acid sequences as shown in the respective line in Table I, application no. 1, columns 5 or 8, preferably the coding region thereof, or homologs or fragments thereof, e.g. if for the nucleic acid molecule in the respective line in column 6 of Table I the term “plastidic” is indicated, used in the inventive process are transformed into plastids, which are metabolical active. Those plastids should preferably maintain at a high copy number in the plant or plant tissue of interest, most preferably the chloroplasts found in green plant tissues, such as leaves or cotyledons or in seeds.

For a good expression in the plastids the nucleic acid sequences as shown in The respective line in Table I, application no. 1, columns 5 or 8, preferably the coding region thereof, or homologs or fargments thereof, e.g. if for the nucleic acid molecule in the respective line in column 6 of Table I the term “plastidic” is indicated, are introduced into an expression cassette using preferably a promoter and terminater, which are active in plastids preferably a chloroplast promoter. Examples of such promoters include the psbA promoter from the gene from spinach or pea, the rbcL promoter, and the atpB promoter from corn.

In another embodiment, an activity disclosed herein as being conferred by a polypeptide shown in the respective line in Table II is increased or generated by linking the polypeptide disclosed in the respective line in Table II or a polypeptide conferring the same said activity with an targeting signal as herein described, if in the respective line in column 6 of Table II the term “mitochondrial ” is listed for said polypeptide. For example, the polypeptide described can be linked to the targeting signal shown in Table b.

In these cases the respective organelle is a mitochondrion. However, the remarks made regarding the plastids are applicable in analogy.

In another embodiment, an activity disclosed herein as being conferred by a polypeptide shown in Table II is increased or generated by the polypeptide disclosed in the respective line in Table II or a polypeptide conferring the same said activity without linking said polypeptide shown in the respective line in Table II to a targeting signal, if in the respective line in column 6 of Table II the term “non-targeted” is listed for said polypeptide.

For the purposes of the description of the present invention, the terms “non-targeted” shall indicate, that the nucleic acid of the invention is expressed without the addition of a “non-natural transit peptide encoding sequence”. A “non-natural transit peptide encoding sequence” is a sequence which is not a natural part of a nucleic acid of the invention, e.g. of the nucleic acids depicted in the respective line in Table I column 5 or 8, preferably the coding region thereof, or homologs or fragments thereof, but which is rather added by molecular manipulation steps as for example described in the example under “plastid targeted expression”. However, the terms “non-targeted” shall not exclude a targeted localisation to any cell compartment for the products of the inventive nucleic acid sequences by their naturally occuring sequence properties within the background of the transgenic organism. The subcellular location of the mature polypetide derived from the enclosed sequences can be predicted by a skilled person for the organism (plant) by using software tools like TargetP (Emanuelsson et al., J. Mol. Biol. 300, 1005 (2000)), ChloroP (Emanuelsson et al., Protein Science 8, 978 (1999)) or other predictive software tools (Emanuelsson et al., Nature Protocols 2, 953 (2007)).

Preferably, the process according to the invention further comprises the step of recovering the fine chemical, which is synthesized by the organism from the organism and/or from the culture medium used for the growth or maintenance of the organism. The term “recovering” means the isolation of the fine chemical in different purities, that means on the one hand harvesting of the biological material, which contains the fine chemical without further purification and on the other hand purities of the fine chemical between 5% and 100% purity, preferred purities are in the range of 10% and 99%. In one embodiment, the purities are at least 20%, 30%, 40%, 50%, 60%, 70%, 80%, 90%, 95% or 99%.

Advantageously the process for the production of the fine chemical methionine leads to an enhanced production of the respective fine chemical. The terms “enhanced” or “increase” mean at least a 10%, 20%, 30%, 40% or 50%, preferably at least 60%, 70%, 80%, 90% or 100%, more preferably 150%, 200%, 300%, 400% or 500% higher production of the respective fine chemical methionine in comparison to the wild-type as defined above, e.g. that means in comparison to a non-human organism without the aforementioned modification of the activity of a protein as shown in the respective line in Table II, application no. 1, column 5 or 8, or a fragment or a homolog thereof. The modification of the activity of a protein as shown in the respective line in Table II, application no. 1, column 5 or 8, or a homolog or a fragment thereof, or their combination can be achieved by joining the protein to a respective transit peptide, e.g. if for the respective encoding nucleic acid molecule in column 6 of Table I the term “plastidic” or “mitochondrial” is indicated.

Surprisingly it was found, that the transgenic expression of a protein as shown in the respective line in Table II, application no. 1, column 5, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, conferred a production of or an increase in methionine,respectively, to the transgenic non-human organism as compared to a corresponding non-transformed wild type.

In one embodiment the transgenic expression of a protein as shown in the respective line in Table II, application no. 1, column 5 or 8, or a homolog or a fragment thereof, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, confers a production of or an increase in methionine, respectively, to the transgenic non-human organism as compared to a corresponding non-transformed wild type.

Accordingly, in one embodiment, an increase of methionine in a non-human organism, as compared to a corresponding non-transformed wild type non-human organism, is conferred in the process of the invention, if the activity of a polypeptide showing the activity of a 49747384_SOYBEAN-protein, or if the activity of the polypeptide 49747384_SOYBEAN, preferably represented by SEQ ID NO. 70, or a homolog or fragment thereof, or if the activity of a polypeptide encoded by a nucleic acid molecule comprising the nucleic acid SEQ ID NO. 69, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 69 or polypeptide SEQ ID NO. 70, respectively, or a homolog or a fragment thereof, is increased or generated, or if the activity 49747384_SOYBEAN-protein is increased or generated in a non-human organism, like a microorganism or a plant cell, plant or part thereof, especially with non-targeted localization, For example, an increase of the methionine of at least 1 percent, particularly in a range of 41 to 74-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a At1g09680protein, or if the activity of the polypeptide At1 g09680, preferably represented by SEQ ID NO. 353, or a homolog or fragment thereof, or if the activity of a polypeptide encoded by a nucleic acid molecule comprising the nucleic acid SEQ ID NO. 352, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 352 or polypeptide SEQ ID NO. 353, respectively, or a homolog or a fragment thereof, is increased or generated, or if the activity At1 g09680-protein is increased or generated in a non-human organism, like a microorganism or a plant cell, plant or part thereof, especially with non-targeted localization, For example, an increase of the methionine of at least 1 percent, particularly in a range of 41 to 60-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a DNA-binding protein, or if the activity of the polypeptide At1g14490, preferably represented by SEQ ID NO. 386, or a homolog or fragment thereof, or if the activity of a polypeptide encoded by a nucleic acid molecule comprising the nucleic acid SEQ ID NO. 385, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 385 or polypeptide SEQ ID NO. 386, respectively, or a homolog or a fragment thereof, is increased or generated, or if the activity DNA-binding protein is increased or generated in a non-human organism, like a microorganism or a plant cell, plant or part thereof, especially with non-targeted localization, For example, an increase of the methionine of at least 1 percent, particularly in a range of 38 to 52-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a bifunctional aspartokinase/homoserine dehydrogenase, or if the activity of the polypeptide At1g31230, preferably represented by SEQ ID NO. 628, or a homolog or fragment thereof, or if the activity of a polypeptide encoded by a nucleic acid molecule comprising the nucleic acid SEQ ID NO. 627, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 627 or polypeptide SEQ ID NO. 628, respectively, or a homolog or a fragment thereof, is increased or generated, or if the activity bifunctional aspartokinase/homoserine dehydrogenase is increased or generated in a non-human organism, like a microorganism or a plant cell, plant or part thereof, especially with non-targeted localization, For example, an increase of the methionine of at least 1 percent, particularly in a range of 60 to 306-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a protein phosphatase, or if the activity of the polypeptide At1 g48040, preferably represented by SEQ ID NO. 813, or a homolog or fragment thereof, or if the activity of a polypeptide encoded by a nucleic acid molecule comprising the nucleic acid SEQ ID NO. 812, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 812 or polypeptide SEQ ID NO. 813, respectively, or a homolog or a fragment thereof, is increased or generated, or if the activity protein phosphatase is increased or generated in a non-human organism, like a microorganism or a plant cell, plant or part thereof, especially with non-targeted localization, For example, an increase of the methionine of at least 1 percent, particularly in a range of 47 to 49-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a transcription factor, or if the activity of the polypeptide At1g68320, preferably represented by SEQ ID NO. 1062, or a homolog or fragment thereof, or if the activity of a polypeptide encoded by a nucleic acid molecule comprising the nucleic acid SEQ ID NO. 1061, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 1061 or polypeptide SEQ ID NO. 1062, respectively, or a homolog or a fragment thereof, is increased or generated, or if the activity transcription factor is increased or generated in a non-human organism, like a microorganism or a plant cell, plant or part thereof, especially with non-targeted localization, For example, an increase of the methionine of at least 1 percent, particularly in a range of 38 to 47-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a protein phosphatase, or if the activity of the polypeptide At2g25070, preferably represented by SEQ ID NO. 1299, or a homolog or fragment thereof, or if the activity of a polypeptide encoded by a nucleic acid molecule comprising the nucleic acid SEQ ID NO. 1298, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 1298 or polypeptide SEQ ID NO. 1299, respectively, or a homolog or a fragment thereof, is increased or generated, or if the activity protein phosphatase is increased or generated in a non-human organism, like a microorganism or a plant cell, plant or part thereof, especially with non-targeted localization, For example, an increase of the methionine of at least 1 percent, particularly in a range of 42 to 89-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a At2g45420-protein, or if the activity of the polypeptide At2g45420, preferably represented by SEQ ID NO. 1624, or a homolog or fragment thereof, or if the activity of a polypeptide encoded by a nucleic acid molecule comprising the nucleic acid SEQ ID NO. 1623, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 1623 or polypeptide SEQ

ID NO. 1624, respectively, or a homolog or a fragment thereof, is increased or generated, or if the activity At2g45420-protein is increased or generated in a non-human organism, like a microorganism or a plant cell, plant or part thereof, especially with non-targeted localization, For example, an increase of the methionine of at least 1 percent, particularly in a range of 32 to 37-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a adenosine kinase, or if the activity of the polypeptide At3g09820, preferably represented by SEQ ID NO. 1697, or a homolog or fragment thereof, or if the activity of a polypeptide encoded by a nucleic acid molecule comprising the nucleic acid SEQ ID NO. 1696, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 1696 or polypeptide SEQ ID NO. 1697, respectively, or a homolog or a fragment thereof, is increased or generated, or if the activity adenosine kinase is increased or generated in a non-human organism, like a microorganism or a plant cell, plant or part thereof, especially with non-targeted localization, For example, an increase of the methionine of at least 1 percent, particularly in a range of 53 to 53-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a protein kinase, or if the activity of the polypeptide At3g23000, preferably represented by SEQ ID NO. 1816, or a homolog or fragment thereof, or if the activity of a polypeptide encoded by a nucleic acid molecule comprising the nucleic acid SEQ ID NO. 1815, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 1815 or polypeptide SEQ ID NO. 1816, respectively, or a homolog or a fragment thereof, is increased or generated, or if the activity protein kinase is increased or generated in a non-human organism, like a microorganism or a plant cell, plant or part thereof, especially with non-targeted localization, For example, an increase of the methionine of at least 1 percent, particularly in a range of 43 to 67-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a auxin response factor, or if the activity of the polypeptide At3g61830, preferably represented by SEQ ID NO. 2368, or a homolog or fragment thereof, or if the activity of a polypeptide encoded by a nucleic acid molecule comprising the nucleic acid SEQ ID NO. 2367, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 2367 or polypeptide SEQ ID NO. 2368, respectively, or a homolog or a fragment thereof, is increased or generated, or if the activity auxin response factor is increased or generated in a non-human organism, like a microorganism or a plant cell, plant or part thereof, especially with non-targeted localization, For example, an increase of the methionine of at least 1 percent, particularly in a range of 36 to 147-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a monthiol glutaredoxin, or if the activity of the polypeptide At3g62930, preferably represented by SEQ ID NO. 2574, or a homolog or fragment thereof, or if the activity of a polypeptide encoded by a nucleic acid molecule comprising the nucleic acid SEQ ID NO. 2573, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 2573 or polypeptide SEQ ID NO. 2574, respectively, or a homolog or a fragment thereof, is increased or generated, or if the activity monthiol glutaredoxin is increased or generated in a non-human organism, like a microorganism or a plant cell, plant or part thereof, especially with non-targeted localization, For example, an increase of the methionine of at least 1 percent, particularly in a range of 37 to 63-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, For example, an increase of the methionine of at least 1 percent, particularly in a range of 37 to 77-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a monothiol glutaredoxin, or if the activity of the polypeptide At4g15670, preferably represented by SEQ ID NO. 3280, or a homolog or fragment thereof, or if the activity of a polypeptide encoded by a nucleic acid molecule comprising the nucleic acid SEQ ID NO. 3279, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 3279 or polypeptide SEQ ID NO. 3280, respectively, or a homolog or a fragment thereof, is increased or generated, or if the activity monothiol glutaredoxin is increased or generated in a non-human organism, like a microorganism or a plant cell, plant or part thereof, especially with non-targeted localization, For example, an increase of the methionine of at least 1 percent, particularly in a range of 37 to 52-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a monothiol glutaredoxin, or if the activity of the polypeptide At4g15700, preferably represented by SEQ ID NO. 3655, or a homolog or fragment thereof, or if the activity of a polypeptide encoded by a nucleic acid molecule comprising the nucleic acid SEQ ID NO. 3654, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 3654 or polypeptide SEQ ID NO. 3655, respectively, or a homolog or a fragment thereof, is increased or generated, or if the activity monothiol glutaredoxin is increased or generated in a non-human organism, like a microorganism or a plant cell, plant or part thereof, especially with non-targeted localization, For example, an increase of the methionine of at least 1 percent, particularly in a range of 43 to 84-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a At4g32480-protein, or if the activity of the polypeptide At4g32480, preferably represented by SEQ ID NO. 4041, or a homolog or fragment thereof, or if the activity of a polypeptide encoded by a nucleic acid molecule comprising the nucleic acid SEQ ID NO. 4040, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 4040 or polypeptide SEQ ID NO. 4041, respectively, or a homolog or a fragment thereof, is increased or generated, or if the activity At4g32480-protein is increased or generated in a non-human organism, like a microorganism or a plant cell, plant or part thereof, especially with non-targeted localization, For example, an increase of the methionine of at least 1 percent, particularly in a range of 44 to 184-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a glutaredoxin, or if the activity of the polypeptide At4g33040, preferably represented by SEQ ID NO. 4103, or a homolog or fragment thereof, or if the activity of a polypeptide encoded by a nucleic acid molecule comprising the nucleic acid SEQ ID NO. 4102, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 4102 or polypeptide SEQ ID NO. 4103, respectively, or a homolog or a fragment thereof, is increased or generated, or if the activity glutaredoxin is increased or generated in a non-human organism, like a microorganism or a plant cell, plant or part thereof, especially with non-targeted localization, For example, an increase of the methionine of at least 1 percent, particularly in a range of 34 to 54-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization, For example, an increase of the methionine of at least 1 percent, particularly in a range of 62 to 83-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a monothiol glutaredoxin, or if the activity of the polypeptide At5g18600, preferably represented by SEQ ID NO. 4905, or a homolog or fragment thereof, or if the activity of a polypeptide encoded by a nucleic acid molecule comprising the nucleic acid SEQ ID NO. 4904, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 4904 or polypeptide SEQ ID NO. 4905, respectively, or a homolog or a fragment thereof, is increased or generated, or if the activity monothiol glutaredoxin is increased or generated in a non-human organism, like a microorganism or a plant cell, plant or part thereof, especially with non-targeted localization, For example, an increase of the methionine of at least 1 percent, particularly in a range of 40 to 75-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a protein phosphatase, or if the activity of the polypeptide At5g57050, preferably represented by SEQ ID NO. 5319, or a homolog or fragment thereof, or if the activity of a polypeptide encoded by a nucleic acid molecule comprising the nucleic acid SEQ ID NO. 5318, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 5318 or polypeptide SEQ ID NO. 5319, respectively, or a homolog or a fragment thereof, is increased or generated, or if the activity protein phosphatase is increased or generated in a non-human organism, like a microorganism or a plant cell, plant or part thereof, especially with non-targeted localization, For example, an increase of the methionine of at least 1 percent, particularly in a range of 36 to 45-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a zinc finger protein, or if the activity of the polypeptide At5g64920, preferably represented by SEQ ID NO. 5494, or a homolog or fragment thereof, or if the activity of a polypeptide encoded by a nucleic acid molecule comprising the nucleic acid SEQ ID NO. 5493, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 5493 or polypeptide SEQ ID NO. 5494, respectively, or a homolog or a fragment thereof, is increased or generated, or if the activity zinc finger protein is increased or generated in a non-human organism, like a microorganism or a plant cell, plant or part thereof, especially with non-targeted localization, For example, an increase of the methionine of at least 1 percent, particularly in a range of 34 to 37-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a malic enzyme, or if the activity of the polypeptide AvinDRAFT_{—}1495, preferably represented by SEQ ID NO. 5558, or a homolog or fragment thereof, or if the activity of a polypeptide encoded by a nucleic acid molecule comprising the nucleic acid SEQ ID NO. 5557, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 5557 or polypeptide SEQ ID NO. 5558, respectively, or a homolog or a fragment thereof, is increased or generated, or if the activity malic enzyme is increased or generated in a non-human organism, like a microorganism or a plant cell, plant or part thereof, especially with non-targeted localization, For example, an increase of the methionine of at least 1 percent, particularly in a range of 38 to 42-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a betahydroxylase, or if the activity of the polypeptide AvinDRAFT_{—}2091, preferably represented by SEQ ID NO. 6041, or a homolog or fragment thereof, or if the activity of a polypeptide encoded by a nucleic acid molecule comprising the nucleic acid SEQ ID NO. 6040, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 6040 or polypeptide SEQ ID NO. 6041, respectively, or a homolog or a fragment thereof, is increased or generated, or if the activity beta-hydroxylase is increased or generated in a non-human organism, like a microorganism or a plant cell, plant or part thereof, especially with non-targeted localization, For example, an increase of the methionine of at least 1 percent, particularly in a range of 37 to 39-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a 2-oxoglutarate dehydrogenase E1 subunit, or if the activity of the polypeptide AvinDRAFT_{—}3028, preferably represented by SEQ ID NO. 6076, or a homolog or fragment thereof, or if the activity of a polypeptide encoded by a nucleic acid molecule comprising the nucleic acid SEQ ID NO. 6075, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 6075 or polypeptide SEQ ID NO. 6076, respectively, or a homolog or a fragment thereof, is increased or generated, or if the activity 2-oxoglutarate dehydrogenase E1 subunit is increased or generated in a non-human organism, like a microorganism or a plant cell, plant or part thereof, especially with non-targeted localization, For example, an increase of the methionine of at least 1 percent, particularly in a range of 39 to 55-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a homoserine dehydrogenase, or if the activity of the polypeptide AvinDRAFT_{—}3546, preferably represented by SEQ ID NO. 6269, or a homolog or fragment thereof, or if the activity of a polypeptide encoded by a nucleic acid molecule comprising the nucleic acid SEQ ID NO. 6268, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 6268 or polypeptide SEQ ID NO. 6269, respectively, or a homolog or a fragment thereof, is increased or generated, or if the activity homoserine dehydrogenase is increased or generated in a non-human organism, like a microorganism or a plant cell, plant or part thereof, especially with non-targeted localization, For example, an increase of the methionine of at least 1 percent, particularly in a range of 58 to 172-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a hydrolase, or if the activity of the polypeptide AvinDRAFT_{—}5103, preferably represented by SEQ ID NO. 6511, or a homolog or fragment thereof, or if the activity of a polypeptide encoded by a nucleic acid molecule comprising the nucleic acid SEQ ID NO. 6510, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 6510 or polypeptide SEQ ID NO. 6511, respectively, or a homolog or a fragment thereof, is increased or generated, or if the activity hydrolase is increased or generated in a non-human organism, like a microorganism or a plant cell, plant or part thereof, especially with non-targeted localization, For example, an increase of the methionine of at least 1 percent, particularly in a range of 280 to 288-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a peptidyl-prolyl cis-trans isomerase, or if the activity of the polypeptide AvinDRAFT_{—}6075, preferably represented by SEQ ID NO. 6675, or a homolog or fragment thereof, or if the activity of a polypeptide encoded by a nucleic acid molecule comprising the nucleic acid SEQ ID NO. 6674, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 6674 or polypeptide SEQ ID NO. 6675, respectively, or a homolog or a fragment thereof, is increased or generated, or if the activity peptidyl-prolyl cis-trans isomerase is increased or generated in a non-human organism, like a microorganism or a plant cell, plant or part thereof, especially with non-targeted localization, For example, an increase of the methionine of at least 1 percent, partitularly in a range of 37 to 46-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a b0012-protein, or if the activity of the polypeptide B0012, preferably represented by SEQ ID NO. 6811, or a homolog or fragment thereof, or if the activity of a polypeptide encoded by a nucleic acid molecule comprising the nucleic acid SEQ ID NO. 6810, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 6810 or polypeptide SEQ ID NO. 6811, respectively, or a homolog or a fragment thereof, is increased or generated, or if the activity b0012-protein is increased or generated in a non-human organism, like a microorganism or a plant cell, plant or part thereof, especially with non-targeted localization, For example, an increase of the methionine of at least 1 percent, particularly in a range of 40 to 66-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a acetolactate synthase small subunit, or if the activity of the polypeptide B0078, preferably represented by SEQ ID NO. 6819, or a homolog or fragment thereof, or if the activity of a polypeptide encoded by a nucleic acid molecule comprising the nucleic acid SEQ ID NO. 6818, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 6818 or polypeptide SEQ ID NO. 6819, respectively, or a homolog or a fragment thereof, is increased or generated, or if the activity acetolactate synthase small subunit is increased or generated in a non-human organism, like a microorganism or a plant cell, plant or part thereof, especially with non-targeted localization, For example, an increase of the methionine of at least 1 percent, particularly in a range of 21 to 24-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a serine protease, or if the activity of the polypeptide B0161, preferably represented by SEQ ID NO. 7082, or a homolog or fragment thereof, or if the activity of a polypeptide encoded by a nucleic acid molecule comprising the nucleic acid SEQ ID NO. 7081, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 7081 or polypeptide SEQ ID NO. 7082, respectively, or a homolog or a fragment thereof, is increased or generated, or if the activity serine protease is increased or generated in a non-human organism, like a microorganism or a plant cell, plant or part thereof, especially with non-targeted localization, For example, an increase of the methionine of at least 1 percent, particularly in a range of 71 to 182-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a 2,3-dihydroxyphenylpropionate 1,2-dioxygenase, or if the activity of the polypeptide B0348, preferably represented by SEQ ID NO. 7270, or a homolog or fragment thereof, or if the activity of a polypeptide encoded by a nucleic acid molecule comprising the nucleic acid SEQ ID NO. 7269, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 7269 or polypeptide SEQ ID NO. 7270, respectively, or a homolog or a fragment thereof, is increased or generated, or if the activity 2,3-dihydroxyphenylpropionate 1,2-dioxygenase is increased or generated in a non-human organism, like a microorganism or a plant cell, plant or part thereof, especially with plastidic localization, For example, an increase of the methionine of at least 1 percent, particularly in a range of 21 to 57-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a ATP-binding component of a transport system, or if the activity of the polypeptide B0449, preferably represented by SEQ ID NO. 7334, or a homolog or fragment thereof, or if the activity of a polypeptide encoded by a nucleic acid molecule comprising the nucleic acid SEQ ID NO. 7333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 7333 or polypeptide SEQ ID NO. 7334, respectively, or a homolog or a fragment thereof, is increased or generated, or if the activity ATP-binding component of a transport system is increased or generated in a non-human organism, like a microorganism or a plant cell, plant or part thereof, especially with non-targeted localization, For example, an increase of the methionine of at least 1 percent, partitularly in a range of 48 to 194-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a membrane transport protein, or if the activity of the polypeptide B0486, preferably represented by SEQ ID NO. 7687, or a homolog or fragment thereof, or if the activity of a polypeptide encoded by a nucleic acid molecule comprising the nucleic acid SEQ ID NO. 7686, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 7686 or polypeptide SEQ ID NO. 7687, respectively, or a homolog or a fragment thereof, is increased or generated, or if the activity membrane transport protein is increased or generated in a non-human organism, like a microorganism or a plant cell, plant or part thereof, especially with non-targeted localization, For example, an increase of the methionine of at least 1 percent, particularly in a range of 32 to 59-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a threonine aldolase, or if the activity of the polypeptide B0870, preferably represented by SEQ ID NO. 7731, or a homolog or fragment thereof, or if the activity of a polypeptide encoded by a nucleic acid molecule comprising the nucleic acid SEQ ID NO. 7730, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 7730 or polypeptide SEQ ID NO. 7731, respectively, or a homolog or a fragment thereof, is increased or generated, or if the activity threonine aldolase is increased or generated in a non-human organism, like a microorganism or a plant cell, plant or part thereof, especially with plastidic localization, For example, an increase of the methionine of at least 1 percent, particularly in a range of 17 to 38-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a major facilitator superfamily transporter protein, or if the activity of the polypeptide B0898, preferably represented by SEQ ID NO. 7918, or a homolog or fragment thereof, or if the activity of a polypeptide encoded by a nucleic acid molecule comprising the nucleic acid SEQ ID NO. 7917, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 7917 or polypeptide SEQ ID NO. 7918, respectively, or a homolog or a fragment thereof, is increased or generated, or if the activity major facilitator superfamily transporter protein is increased or generated in a non-human organism, like a microorganism or a plant cell, plant or part thereof, especially with non-targeted localization, For example, an increase of the methionine of at least 1 percent, particularly in a range of 36 to 1310-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a b1003-protein, or if the activity of the polypeptide B1003, preferably represented by SEQ ID NO. 7942, or a homolog or fragment thereof, or if the activity of a polypeptide encoded by a nucleic acid molecule comprising the nucleic acid SEQ ID NO. 7941, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 7941 or polypeptide SEQ ID NO. 7942, respectively, or a homolog or a fragment thereof, is increased or generated, or if the activity b1003-protein is increased or generated in a non-human organism, like a microorganism or a plant cell, plant or part thereof, especially with non-targeted localization, For example, an increase of the methionine of at least 1 percent, particularly in a range of 49 to 144-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a b1522-protein, or if the activity of the polypeptide B1522, preferably represented by SEQ ID NO. 7948, or a homolog or fragment thereof, or if the activity of a polypeptide encoded by a nucleic acid molecule comprising the nucleic acid SEQ ID NO. 7947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 7947 or polypeptide SEQ ID NO. 7948, respectively, or a homolog or a fragment thereof, is increased or generated, or if the activity b1522-protein is increased or generated in a non-human organism, like a microorganism or a plant cell, plant or part thereof, especially with non-targeted localization, For example, an increase of the methionine of at least 1 percent, particularly in a range of 42 to 144-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a transport protein, or if the activity of the polypeptide B1601, preferably represented by SEQ ID NO. 7993, or a homolog or fragment thereof, or if the activity of a polypeptide encoded by a nucleic acid molecule comprising the nucleic acid SEQ ID NO. 7992, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 7992 or polypeptide SEQ ID NO. 7993, respectively, or a homolog or a fragment thereof, is increased or generated, or if the activity transport protein is increased or generated in a non-human organism, like a microorganism or a plant cell, plant or part thereof, especially with non-targeted localization, For example, an increase of the methionine of at least 1 percent, particularly in a range of 41 to 64-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a L-serine dehydratase, or if the activity of the polypeptide B1814, preferably represented by SEQ ID NO. 8034, or a homolog or fragment thereof, or if the activity of a polypeptide encoded by a nucleic acid molecule comprising the nucleic acid SEQ ID NO. 8033, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 8033 or polypeptide SEQ ID NO. 8034, respectively, or a homolog or a fragment thereof, is increased or generated, or if the activity L-serine dehydratase is increased or generated in a non-human organism, like a microorganism or a plant cell, plant or part thereof, especially with plastidic localization, For example, an increase of the methionine of at least 1 percent, particularly in a range of 19 to 59-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a serine/threonine-protein phosphatase, or if the activity of the polypeptide B1838, preferably represented by SEQ ID NO. 8317, or a homolog or fragment thereof, or if the activity of a polypeptide encoded by a nucleic acid molecule comprising the nucleic acid SEQ ID NO. 8316, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 8316 or polypeptide SEQ ID NO. 8317, respectively, or a homolog or a fragment thereof, is increased or generated, or if the activity serine/threonine-protein phosphatase is increased or generated in a non-human organism, like a microorganism or a plant cell, plant or part thereof, especially with non-targeted localization, For example, an increase of the methionine of at least 1 percent, particularly in a range of 50 to 59-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a pyruvate kinase, or if the activity of the polypeptide B1854, preferably represented by SEQ ID NO. 8364, or a homolog or fragment thereof, or if the activity of a polypeptide encoded by a nucleic acid molecule comprising the nucleic acid SEQ ID NO. 8363, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 8363 or polypeptide SEQ ID NO. 8364, respectively, or a homolog or a fragment thereof, is increased or generated, or if the activity pyruvate kinase is increased or generated in a non-human organism, like a microorganism or a plant cell, plant or part thereof, especially with plastidic localization, For example, an increase of the methionine of at least 1 percent, particularly in a range of 19 to 45-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a b2032-protein, or if the activity of the polypeptide B2032, preferably represented by SEQ ID NO. 8921, or a homolog or fragment thereof, or if the activity of a polypeptide encoded by a nucleic acid molecule comprising the nucleic acid SEQ ID NO. 8920, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 8920 or polypeptide SEQ ID NO. 8921, respectively, or a homolog or a fragment thereof, is increased or generated, or if the activity b2032-protein is increased or generated in a non-human organism, like a microorganism or a plant cell, plant or part thereof, especially with non-targeted localization, For example, an increase of the methionine of at least 1 percent, particularly in a range of 37 to 75-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a uridine/cytidine kinase, or if the activity of the polypeptide B2066, preferably represented by SEQ ID NO. 8938, or a homolog or fragment thereof, or if the activity of a polypeptide encoded by a nucleic acid molecule comprising the nucleic acid SEQ ID NO. 8937, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 8937 or polypeptide SEQ ID NO. 8938, respectively, or a homolog or a fragment thereof, is increased or generated, or if the activity uridine/cytidine kinase is increased or generated in a non-human organism, like a microorganism or a plant cell, plant or part thereof, especially with mitochondrial localization, For example, an increase of the methionine of at least 1 percent, particularly in a range of 35 to 64-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a b2345-protein, or if the activity of the polypeptide B2345, preferably represented by SEQ ID NO. 9157, or a homolog or fragment thereof, or if the activity of a polypeptide encoded by a nucleic acid molecule comprising the nucleic acid SEQ ID NO. 9156, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 9156 or polypeptide SEQ ID NO. 9157, respectively, or a homolog or a fragment thereof, is increased or generated, or if the activity b2345-protein is increased or generated in a non-human organism, like a microorganism or a plant cell, plant or part thereof, especially with non-targeted localization, For example, an increase of the methionine of at least 1 percent, particularly in a range of 41 to 231-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a b2513-protein, or if the activity of the polypeptide B2513, preferably represented by SEQ ID NO. 9168, or a homolog or fragment thereof, or if the activity of a polypeptide encoded by a nucleic acid molecule comprising the nucleic acid SEQ ID NO. 9167, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 9167 or polypeptide SEQ ID NO. 9168, respectively, or a homolog or a fragment thereof, is increased or generated, or if the activity b2513-protein is increased or generated in a non-human organism, like a microorganism or a plant cell, plant or part thereof, especially with non-targeted localization, For example, an increase of the methionine of at least 1 percent, particularly in a range of 39 to 55-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a b2673-protein, or if the activity of the polypeptide B2673, preferably represented by SEQ ID NO. 9245, or a homolog or fragment thereof, or if the activity of a polypeptide encoded by a nucleic acid molecule comprising the nucleic acid SEQ ID NO. 9244, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 9244 or polypeptide SEQ ID NO. 9245, respectively, or a homolog or a fragment thereof, is increased or generated, or if the activity b2673-protein is increased or generated in a non-human organism, like a microorganism or a plant cell, plant or part thereof, especially with non-targeted localization, For example, an increase of the methionine of at least 1 percent, particularly in a range of 32 to 57-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a arginine exporter protein, or if the activity of the polypeptide B2923, preferably represented by SEQ ID NO. 9334, or a homolog or fragment thereof, or if the activity of a polypeptide encoded by a nucleic acid molecule comprising the nucleic acid SEQ ID NO. 9333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 9333 or polypeptide SEQ ID NO. 9334, respectively, or a homolog or a fragment thereof, is increased or generated, or if the activity arginine exporter protein is increased or generated in a non-human organism, like a microorganism or a plant cell, plant or part thereof, especially with non-targeted localization, For example, an increase of the methionine of at least 1 percent, particularly in a range of 32 to 54-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a b3246-protein, or if the activity of the polypeptide B3246, preferably represented by SEQ ID NO. 9471, or a homolog or fragment thereof, or if the activity of a polypeptide encoded by a nucleic acid molecule comprising the nucleic acid SEQ ID NO. 9470, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 9470 or polypeptide SEQ ID NO. 9471, respectively, or a homolog or a fragment thereof, is increased or generated, or if the activity b3246-protein is increased or generated in a non-human organism, like a microorganism or a plant cell, plant or part thereof, especially with non-targeted localization, For example, an increase of the methionine of at least 1 percent, particularly in a range of 36 to 46-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a acetyl CoA carboxylase, or if the activity of the polypeptide B3256, preferably represented by SEQ ID NO. 9493, or a homolog or fragment thereof, or if the activity of a polypeptide encoded by a nucleic acid molecule comprising the nucleic acid SEQ ID NO. 9492, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 9492 or polypeptide SEQ ID NO. 9493, respectively, or a homolog or a fragment thereof, is increased or generated, or if the activity acetyl CoA carboxylase is increased or generated in a non-human organism, like a microorganism or a plant cell, plant or part thereof, especially with non-targeted localization, For example, an increase of the methionine of at least 1 percent, particularly in a range of 77 to 121-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a b3346-protein, or if the activity of the polypeptide B3346, preferably represented by SEQ ID NO. 10105, or a homolog or fragment thereof, or if the activity of a polypeptide encoded by a nucleic acid molecule comprising the nucleic acid SEQ ID NO. 10104, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 10104 or polypeptide SEQ ID NO. 10105, respectively, or a homolog or a fragment thereof, is increased or generated, or if the activity b3346-protein is increased or generated in a non-human organism, like a microorganism or a plant cell, plant or part thereof, especially with non-targeted localization, For example, an increase of the methionine of at least 1 percent, particularly in a range of 37 to 40-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a valine-pyruvate transaminase, or if the activity of the polypeptide B3572, preferably represented by SEQ ID NO. 10173, or a homolog or fragment thereof, or if the activity of a polypeptide encoded by a nucleic acid molecule comprising the nucleic acid SEQ ID NO. 10172, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 10172 or polypeptide SEQ ID NO. 10173, respectively, or a homolog or a fragment thereof, is increased or generated, or if the activity valine-pyruvate transaminase is increased or generated in a non-human organism, like a microorganism or a plant cell, plant or part thereof, especially with plastidic localization, For example, an increase of the methionine of at least 1 percent, particularly in a range of 20 to 652-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a dihydroxyacid dehydratase, or if the activity of the polypeptide B3771, preferably represented by SEQ ID NO. 10253, or a homolog or fragment thereof, or if the activity of a polypeptide encoded by a nucleic acid molecule comprising the nucleic acid SEQ ID NO. 10252, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 10252 or polypeptide SEQ ID NO. 10253, respectively, or a homolog or a fragment thereof, is increased or generated, or if the activity dihydroxyacid dehydratase is increased or generated in a non-human organism, like a microorganism or a plant cell, plant or part thereof, especially with non-targeted localization, For example, an increase of the methionine of at least 1 percent, particularly in a range of 22 to 93-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a b3817-protein, or if the activity of the polypeptide B3817, preferably represented by SEQ ID NO. 10709, or a homolog or fragment thereof, or if the activity of a polypeptide encoded by a nucleic acid molecule comprising the nucleic acid SEQ ID NO. 10708, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 10708 or polypeptide SEQ ID NO. 10709, respectively, or a homolog or a fragment thereof, is increased or generated, or if the activity b3817-protein is increased or generated in a non-human organism, like a microorganism or a plant cell, plant or part thereof, especially with non-targeted localization, For example, an increase of the methionine of at least 1 percent, particularly in a range of 37 to 61-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a threonine efflux protein, or if the activity of the polypeptide B3823, preferably represented by SEQ ID NO. 10727, or a homolog or fragment thereof, or if the activity of a polypeptide encoded by a nucleic acid molecule comprising the nucleic acid SEQ ID NO. 10726, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 10726 or polypeptide SEQ ID NO. 10727, respectively, or a homolog or a fragment thereof, is increased or generated, or if the activity threonine efflux protein is increased or generated in a non-human organism, like a microorganism or a plant cell, plant or part thereof, especially with non-targeted localization, For example, an increase of the methionine of at least 1 percent, particularly in a range of 38 to 48-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a b4029-protein, or if the activity of the polypeptide B4029, preferably represented by SEQ ID NO. 10741, or a homolog or fragment thereof, or if the activity of a polypeptide encoded by a nucleic acid molecule comprising the nucleic acid SEQ ID NO. 10740, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 10740 or polypeptide SEQ ID NO. 10741, respectively, or a homolog or a fragment thereof, is increased or generated, or if the activity b4029-protein is increased or generated in a non-human organism, like a microorganism or a plant cell, plant or part thereof, especially with non-targeted localization, For example, an increase of the methionine of at least 1 percent, particularly in a range of 84 to 691-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a glutaredoxin, or if the activity of the polypeptide GM02LC12622, preferably represented by SEQ ID NO. 10812, or a homolog or fragment thereof, or if the activity of a polypeptide encoded by a nucleic acid molecule comprising the nucleic acid SEQ ID NO. 10811, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 10811 or polypeptide SEQ ID NO. 10812, respectively, or a homolog or a fragment thereof, is increased or generated, or if the activity glutaredoxin is increased or generated in a non-human organism, like a microorganism or a plant cell, plant or part thereof, especially with non-targeted localization, For example, an increase of the methionine of at least 1 percent, particularly in a range of 37 to 55-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a 5′-nucleotidase, or if the activity of the polypeptide Sll1108, preferably represented by SEQ ID NO. 11212, or a homolog or fragment thereof, or if the activity of a polypeptide encoded by a nucleic acid molecule comprising the nucleic acid SEQ ID NO. 11211, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 11211 or polypeptide SEQ ID NO. 11212, respectively, or a homolog or a fragment thereof, is increased or generated, or if the activity 5′-nucleotidase is increased or generated in a non-human organism, like a microorganism or a plant cell, plant or part thereof, especially with mitochondrial localization, For example, an increase of the methionine of at least 1 percent, particularly in a range of 39 to 67-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a glutathione S-transferase, or if the activity of the polypeptide Sll1545, preferably represented by SEQ ID NO. 11424, or a homolog or fragment thereof, or if the activity of a polypeptide encoded by a nucleic acid molecule comprising the nucleic acid SEQ ID NO. 11423, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 11423 or polypeptide SEQ ID NO. 11424, respectively, or a homolog or a fragment thereof, is increased or generated, or if the activity glutathione S-transferase is increased or generated in a non-human organism, like a microorganism or a plant cell, plant or part thereof, especially with mitochondrial localization, For example, an increase of the methionine of at least 1 percent, particularly in a range of 38 to 58-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a coproporphyrinogen III oxidase, or if the activity of the polypeptide Sll1917, preferably represented by SEQ ID NO. 11472, or a homolog or fragment thereof, or if the activity of a polypeptide encoded by a nucleic acid molecule comprising the nucleic acid SEQ ID NO. 11471, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 11471 or polypeptide SEQ ID NO. 11472, respectively, or a homolog or a fragment thereof, is increased or generated, or if the activity coproporphyrinogen III oxidase is increased or generated in a non-human organism, like a microorganism or a plant cell, plant or part thereof, especially with mitochondrial localization, For example, an increase of the methionine of at least 1 percent, particularly in a range of 30 to 34-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a oxidoreductase, or if the activity of the polypeptide Slr0338, preferably represented by SEQ ID NO. 11991, or a homolog or fragment thereof, or if the activity of a polypeptide encoded by a nucleic acid molecule comprising the nucleic acid SEQ ID NO. 11990, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 11990 or polypeptide SEQ ID NO. 11991, respectively, or a homolog or a fragment thereof, is increased or generated, or if the activity oxidoreductase is increased or generated in a non-human organism, like a microorganism or a plant cell, plant or part thereof, especially with non-targeted localization, For example, an increase of the methionine of at least 1 percent, particularly in a range of 29 to 40-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a oxidoreductase, or if the activity of the polypeptide Slr0338, preferably represented by SEQ ID NO. 11991, or a homolog or fragment thereof, or if the activity of a polypeptide encoded by a nucleic acid molecule comprising the nucleic acid SEQ ID NO. 11990, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 11990 or polypeptide SEQ ID NO. 11991, respectively, or a homolog or a fragment thereof, is increased or generated, or if the activity oxidoreductase is increased or generated in a non-human organism, like a microorganism or a plant cell, plant or part thereof, especially with plastidic localization, For example, an increase of the methionine of at least 1 percent, particularly in a range of 27 to 37-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a Photosystem I reaction center subunit XI, or if the activity of the polypeptide Slr1655, preferably represented by SEQ ID NO. 12071, or a homolog or fragment thereof, or if the activity of a polypeptide encoded by a nucleic acid molecule comprising the nucleic acid SEQ ID NO. 12070, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 12070 or polypeptide SEQ ID NO. 12071, respectively, or a homolog or a fragment thereof, is increased or generated, or if the activity Photosystem I reaction center subunit XI is increased or generated in a non-human organism, like a microorganism or a plant cell, plant or part thereof, especially with mitochondrial localization, For example, an increase of the methionine of at least 1 percent, particularly in a range of 30 to 58-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a phosphoadenosine phosphosulfate reductase , or if the activity of the polypeptide Slr1791, preferably represented by SEQ ID NO. 12141, or a homolog or fragment thereof, or if the activity of a polypeptide encoded by a nucleic acid molecule comprising the nucleic acid SEQ ID NO. 12140, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 12140 or polypeptide SEQ ID NO. 12141, respectively, or a homolog or a fragment thereof, is increased or generated, or if the activity phosphoadenosine phosphosulfate reductase is increased or generated in a non-human organism, like a microorganism or a plant cell, plant or part thereof, especially with plastidic localization, For example, an increase of the methionine of at least 1 percent, particularly in a range of 38 to 66-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a threonine dehydratase, or if the activity of the polypeptide Slr2072, preferably represented by SEQ ID NO. 12342, or a homolog or fragment thereof, or if the activity of a polypeptide encoded by a nucleic acid molecule comprising the nucleic acid SEQ ID NO. 12341, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 12341 or polypeptide SEQ ID NO. 12342, respectively, or a homolog or a fragment thereof, is increased or generated, or if the activity threonine dehydratase is increased or generated in a non-human organism, like a microorganism or a plant cell, plant or part thereof, especially with plastidic localization, For example, an increase of the methionine of at least 1 percent, particularly in a range of 36 to 74-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a Sec-independent protein translocase subunit, or if the activity of the polypeptide TTC0019, preferably represented by SEQ ID NO. 12699, or a homolog or fragment thereof, or if the activity of a polypeptide encoded by a nucleic acid molecule comprising the nucleic acid SEQ ID NO. 12698, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 12698 or polypeptide SEQ ID NO. 12699, respectively, or a homolog or a fragment thereof, is increased or generated, or if the activity Sec-independent protein translocase subunit is increased or generated in a non-human organism, like a microorganism or a plant cell, plant or part thereof, especially with non-targeted localization, For example, an increase of the methionine of at least 1 percent, particularly in a range of 35 to 71-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a homocitrate synthase, or if the activity of the polypeptide TTC1550, preferably represented by SEQ ID NO. 12975, or a homolog or fragment thereof, or if the activity of a polypeptide encoded by a nucleic acid molecule comprising the nucleic acid SEQ ID NO. 12974, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 12974 or polypeptide SEQ ID NO. 12975, respectively, or a homolog or a fragment thereof, is increased or generated, or if the activity homocitrate synthase is increased or generated in a non-human organism, like a microorganism or a plant cell, plant or part thereof, especially with non-targeted localization, For example, an increase of the methionine of at least 1 percent, particularly in a range of 36 to 44-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a cystathioninelyase, or if the activity of the polypeptide Yal012w, preferably represented by SEQ ID NO. 13377, or a homolog or fragment thereof, or if the activity of a polypeptide encoded by a nucleic acid molecule comprising the nucleic acid SEQ ID NO. 13376, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 13376 or polypeptide SEQ ID NO. 13377, respectively, or a homolog or a fragment thereof, is increased or generated, or if the activity cystathionine-lyase is increased or generated in a non-human organism, like a microorganism or a plant cell, plant or part thereof, especially with non-targeted localization, For example, an increase of the methionine of at least 1 percent, particularly in a range of 22 to 31-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a cyclin, or if the activity of the polypeptide Ydl155w, preferably represented by SEQ ID NO. 14172, or a homolog or fragment thereof, or if the activity of a polypeptide encoded by a nucleic acid molecule comprising the nucleic acid SEQ ID NO. 14171, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 14171 or polypeptide SEQ ID NO. 14172, respectively, or a homolog or a fragment thereof, is increased or generated, or if the activity cyclin is increased or generated in a non-human organism, like a microorganism or a plant cell, plant or part thereof, especially with non-targeted localization, For example, an increase of the methionine of at least 1 percent, particularly in a range of 34 to 107-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a protein kinase, or if the activity of the polypeptide Ydl159w, preferably represented by SEQ ID NO. 14276, or a homolog or fragment thereof, or if the activity of a polypeptide encoded by a nucleic acid molecule comprising the nucleic acid SEQ ID NO. 14275, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 14275 or polypeptide SEQ ID NO. 14276, respectively, or a homolog or a fragment thereof, is increased or generated, or if the activity protein kinase is increased or generated in a non-human organism, like a microorganism or a plant cell, plant or part thereof, especially with plastidic localization, For example, an increase of the methionine of at least 1 percent, particularly in a range of 44 to 54-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a protein kinase, or if the activity of the polypeptide Ydl168w, preferably represented by SEQ ID NO. 14303, or a homolog or fragment thereof, or if the activity of a polypeptide encoded by a nucleic acid molecule comprising the nucleic acid SEQ ID NO. 14302, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 14302 or polypeptide SEQ ID NO. 14303, respectively, or a homolog or a fragment thereof, is increased or generated, or if the activity protein kinase is increased or generated in a non-human organism, like a microorganism or a plant cell, plant or part thereof, especially with plastidic localization, For example, an increase of the methionine of at least 1 percent, particularly in a range of 40 to 46-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a F-box protein, or if the activity of the polypeptide Ydr131c, preferably represented by SEQ ID NO. 14707, or a homolog or fragment thereof, or if the activity of a polypeptide encoded by a nucleic acid molecule comprising the nucleic acid SEQ ID NO. 14706, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 14706 or polypeptide SEQ ID NO. 14707, respectively, or a homolog or a fragment thereof, is increased or generated, or if the activity F-box protein is increased or generated in a non-human organism, like a microorganism or a plant cell, plant or part thereof, especially with plastidic localization, For example, an increase of the methionine of at least 1 percent, particularly in a range of 30 to 31-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a yhl013c-protein, or if the activity of the polypeptide Yhl013c, preferably represented by SEQ ID NO. 14716, or a homolog or fragment thereof, or if the activity of a polypeptide encoded by a nucleic acid molecule comprising the nucleic acid SEQ ID NO. 14715, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 14715 or polypeptide SEQ ID NO. 14716, respectively, or a homolog or a fragment thereof, is increased or generated, or if the activity yhl013c-protein is increased or generated in a non-human organism, like a microorganism or a plant cell, plant or part thereof, especially with plastidic localization, For example, an increase of the methionine of at least 1 percent, particularly in a range of 32 to 73-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a (DL)-glycerol-3-phosphatase, or if the activity of the polypeptide Yi1053w, preferably represented by SEQ ID NO. 14770, or a homolog or fragment thereof, or if the activity of a polypeptide encoded by a nucleic acid molecule comprising the nucleic acid SEQ ID NO. 14769, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 14769 or polypeptide SEQ ID NO. 14770, respectively, or a homolog or a fragment thereof, is increased or generated, or if the activity (DL)-glycerol-3-phosphatase is increased or generated in a non-human organism, like a microorganism or a plant cell, plant or part thereof, especially with plastidic localization, For example, an increase of the methionine of at least 1 percent, particularly in a range of 26 to 31-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a glycogenin, or if the activity of the polypeptide Yjl137c, preferably represented by SEQ ID NO. 14822, or a homolog or fragment thereof, or if the activity of a polypeptide encoded by a nucleic acid molecule comprising the nucleic acid SEQ ID NO. 14821, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 14821 or polypeptide SEQ ID NO. 14822, respectively, or a homolog or a fragment thereof, is increased or generated, or if the activity glycogenin is increased or generated in a non-human organism, like a microorganism or a plant cell, plant or part thereof, especially with plastidic localization, For example, an increase of the methionine of at least 1 percent, particularly in a range of 26 to 37 -percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a cystathionine gamma-synthase, or if the activity of the polypeptide Yjr130c, preferably represented by SEQ ID NO. 14844, or a homolog or fragment thereof, or if the activity of a polypeptide encoded by a nucleic acid molecule comprising the nucleic acid SEQ ID NO. 14843, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 14843 or polypeptide SEQ ID NO. 14844, respectively, or a homolog or a fragment thereof, is increased or generated, or if the activity cystathionine gamma-synthase is increased or generated in a non-human organism, like a microorganism or a plant cell, plant or part thereof, especially with non-targeted localization, For example, an increase of the methionine of at least 1 percent, particularly in a range of 24 to 30-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a homoserine dehydrogenase, or if the activity of the polypeptide Yjr139c, preferably represented by SEQ ID NO. 14886, or a homolog or fragment thereof, or if the activity of a polypeptide encoded by a nucleic acid molecule comprising the nucleic acid SEQ ID NO. 14885, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 14885 or polypeptide SEQ ID NO. 14886, respectively, or a homolog or a fragment thereof, is increased or generated, or if the activity homoserine dehydrogenase is increased or generated in a non-human organism, like a microorganism or a plant cell, plant or part thereof, especially with plastidic localization, For example, an increase of the methionine of at least 1 percent, particularly in a range of 38 to 59-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a yml084w-protein, or if the activity of the polypeptide Yml084w, preferably represented by SEQ ID NO. 15176, or a homolog or fragment thereof, or if the activity of a polypeptide encoded by a nucleic acid molecule comprising the nucleic acid SEQ ID NO. 15175, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 15175 or polypeptide SEQ ID NO. 15176, respectively, or a homolog or a fragment thereof, is increased or generated, or if the activity yml084w-protein is increased or generated in a non-human organism, like a microorganism or a plant cell, plant or part thereof, especially with plastidic localization, For example, an increase of the methionine of at least 1 percent, particularly in a range of 31 to 52-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a yol160w-protein, or if the activity of the polypeptide Yol160w, preferably represented by SEQ ID NO. 15180, or a homolog or fragment thereof, or if the activity of a polypeptide encoded by a nucleic acid molecule comprising the nucleic acid SEQ ID NO. 15179, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 15179 or polypeptide SEQ ID NO. 15180, respectively, or a homolog or a fragment thereof, is increased or generated, or if the activity yol160w-protein is increased or generated in a non-human organism, like a microorganism or a plant cell, plant or part thereof, especially with plastidic localization, For example, an increase of the methionine of at least 1 percent, particularly in a range of 31 to 47-percent is conferred as compared to a corresponding non-transformed wild type non-human organism.

Accordingly, in one embodiment, an increase of methionine in a non-human organism, as compared to a corresponding non-transformed wild type non-human organism, is conferred in the process of the invention, if the activity of a polypeptide showing the activity of a yor392w-protein, or if the activity of the polypeptide Yor392w, preferably represented by SEQ ID NO. 15184, or a homolog or fragment thereof, or if the activity of a polypeptide encoded by a nucleic acid molecule comprising the nucleic acid SEQ ID NO. 15183, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 1, column 5 or 8 in the respective same line as the nucleic acid molecule SEQ ID NO. 15183 or polypeptide SEQ ID NO. 15184, respectively, or a homolog or a fragment thereof, is increased or generated, or if the activity yor392w-protein is increased or generated in a non-human organism, like a microorganism or a plant cell, plant or part thereof, especially with plastidic localization, For example, an increase of the methionine of at least 1 percent, particularly in a range of 14 to 20-percent is conferred as compared to a corresponding non-transformed wild type non-human organism.

In an embodiment of the invention, a protein or polypeptide has the “activity of a protein as shown in the respective line in Table II, column 3” if its de novo activity, or its increased expression directly or indirectly leads to an increase in the fine chemical level in the organism or a part thereof, preferably in a cell of said organism, and the protein has the above-mentioned activities of a protein as shown in the respective line in Table II, column 3. Throughout the specification the activity or preferably the biological activity of such a protein or polypeptide or an nucleic acid molecule or sequence encoding such protein or polypeptide is identical or similar if it still has the biological or enzymatic activity of a protein as shown in the respective line in Table II, column 3, or which has at least 10% of the original biological or enzymatic activity, preferably at least 20%, 30%, 40%, 50%, 60%, 70%, 80%, 90%, 95% or 99% in comparison to a protein as shown in Table II, column 3.

A series of mechanisms exists via which a modification of a protein, e.g. the polypeptide of the invention can directly or indirectly affect the yield, production and/or production efficiency of the fine chemical.

For example, the molecule number or the specific activity of the polypeptide or the nucleic acid molecule may be increased. Larger amounts of the fine chemical can be produced if the polypeptide or the nucleic acid of the invention is expressed de novo in an organism lacking the activity of said protein, preferably the nucleic acid molecules as mentioned in the respective line in Table I, columns 5 or 8, preferably the coding region thereof, or a homolog or a fragment thereof, alone or, if desired in combination with a transit peptide for example as mentioned in Table a or b, respectively, or in another embodiment by introducing said nucleic acid molecules into an organelle, such as a plastid, in the transgenic organism. However, it is also possible to modify the expression of the gene which is naturally present in the organisms, for example by integrating a nucleic acid sequence, encoding an organelle targeting sequence in front (5 prime) of the coding sequence, leading to a functional preprotein, which is directed for example to the organelle.

This also applies analogously to the combined increased expression of the nucleic acid molecule of the present invention or its gene product with that of further enzymes or regulators of the fine chemical biosynthesis pathways, e.g. which are useful for the synthesis of the fine chemicals.

The increase or modulation according to this invention can be constitutive, e.g. due to a stable permanent transgenic expression or to a stable mutation in the corresponding endogenous gene encoding the nucleic acid molecule of the invention or to a modulation of the expression or of the behaviour of a gene conferring the expression of the polypeptide of the invention, or transient, e.g. due to a transient transformation or temporary addition of a modulator such as an agonist or antagonist or inducible, e.g. after transformation with a inducible construct carrying the nucleic acid molecule of the invention under control of an inducible promoter and adding the inducer, e.g. tetracycline or as described herein below.

The increase in activity of the polypeptide amounts in a cell, an organelle, an organ or a non-human organism or a part thereof preferably to at least 1%, 5%, 10%, 20%, 50%, 70%, 80%, 90%, 100%, 150% or 200%, 250% or more in comparison to the control, reference or wild type.

In an embodiment the increase in activity of the polypeptide amounts in an organelle such as a plastid or a mitochondrion. In another embodiment the increase in activity of the polypeptide amounts in the cytoplasm. In another embodiment the increase in activity of the polypeptide amounts in the cytosol.

In the context of the application the term “cytoplasm” defines the compartment of the cell to which the protein is directed by the host cell without the addition of artifical targeting sequences, which are naturally not part of the protein.

In the context of the application the term “cytosol” defines the fluid of the cell between the plasma membrane and the organelles; that means that the organelles as well as the nucleus are not part of the cytosol.

The specific activity of a polypeptide encoded by a nucleic acid molecule of the present invention or of the polypeptide of the present invention can be tested as described in the examples. In particular, the expression of a protein in question in a cell, e.g. a plant cell or a microorganism and the detection of an increase the fine chemical level in comparison to a control is an easy test and can be performed as described in the state of the art.

The nucleic acid sequence of 49747384_SOYBEAN from Glycine max, e.g. as shown in the respective line in column 5 of Table I, application no. 1 is unpublished. And the activity of the gene product thereof is the activity of 49747384_SOYBEAN-protein.

Accordingly, in one embodiment, the process of the present invention for producing methionine in a non-human organism, like a microorganism or a plant or a part thereof, comprises increasing or generating the activity of a gene product with the activity of a gene product conferring the activity of “49747384_SOYBEAN-protein”, especially from Glycine max or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said 49747384_SOYBEAN, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said 49747384_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said 49747384_SOYBEAN, or a functional equivalent or a homolog thereof as depicted in column 8 of Table II, application no. 1, preferably a homolog or functional equivalent as depicted in column 8 of Table II B, application no. 1, and being depicted in the same respective line as said 49747384_SOYBEAN, and preferably the activity is increased non-targeted.

Accordingly, in one embodiment, the molecule which activity is to be increased in the process of the invention is the gene product with an activity as a “49747384_SOYBEAN-protein”, preferably it is the molecule of section (a) or (b) of this paragraph.

In particular, it was observed that in plants, especially in Arabidopsis thaliana, increasing or generating the activity of a gene product non-targeted with the activity of a “49747384_SOYBEAN-protein”, preferably being encoded by a gene comprising the nucleic acid sequence SEQ ID NO. 69, preferably the coding region thereof, conferred the production of or the increase in methionine compared with the wild type control.

The nucleic acid sequence of At1g09680 from Arabidopsis thaliana, e.g. as shown in the respective line in column 5 of Table I, application no. 1 is unpublished. And the activity of the gene product thereof is the activity of At1 g09680-protein.

Accordingly, in one embodiment, the process of the present invention for producing methionine in a non-human organism, like a microorganism or a plant or a part thereof, comprises increasing or generating the activity of a gene product with the activity of a gene product conferring the activity of “At1g09680-protein”, especially from Arabidopsis thaliana or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said At1g09680, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said At1g09680, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said At1g09680, or a functional equivalent or a homolog thereof as depicted in column 8 of Table II, application no. 1, preferably a homolog or functional equivalent as depicted in column 8 of Table II B, application no. 1, and being depicted in the same respective line as said At1g09680, and preferably the activity is increased non-targeted.

Accordingly, in one embodiment, the molecule which activity is to be increased in the process of the invention is the gene product with an activity as a “At1g09680-protein”, preferably it is the molecule of section (a) or (b) of this paragraph.

In particular, it was observed that in plants, especially in Arabidopsis thaliana, increasing or generating the activity of a gene product non-targeted with the activity of a “At1g09680-protein”, preferably being encoded by a gene comprising the nucleic acid sequence SEQ ID NO. 352, preferably the coding region thereof, conferred the production of or the increase in methionine compared with the wild type control.

The nucleic acid sequence of At1g14490 from Arabidopsis thaliana, e.g. as shown in the respective line in column 5 of Table I, application no. 1 is unpublished. And the activity of the gene product thereof is the activity of DNA-binding protein.

Accordingly, in one embodiment, the process of the present invention for producing methionine in a non-human organism, like a microorganism or a plant or a part thereof, comprises increasing or generating the activity of a gene product with the activity of a gene product conferring the activity of “DNA-binding protein”, especially from Arabidopsis thaliana or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said At1g14490, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said At1g14490, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said At1g14490, or a functional equivalent or a homolog thereof as depicted in column 8 of Table II, application no. 1, preferably a homolog or functional equivalent as depicted in column 8 of Table II B, application no. 1, and being depicted in the same respective line as said At1g14490, and preferably the activity is increased non-targeted.

Accordingly, in one embodiment, the molecule which activity is to be increased in the process of the invention is the gene product with an activity as a “DNA-binding protein”, preferably it is the molecule of section (a) or (b) of this paragraph.

In particular, it was observed that in plants, especially in Arabidopsis thaliana, increasing or generating the activity of a gene product non-targeted with the activity of a “DNA-binding protein”, preferably being encoded by a gene comprising the nucleic acid sequence SEQ ID NO. 385, preferably the coding region thereof, conferred the production of or the increase in methionine compared with the wild type control.

The nucleic acid sequence of At1g31230 from Arabidopsis thaliana, e.g. as shown in the respective line in column 5 of Table I, application no. 1 is unpublished. And the activity of the gene product thereof is the activity of bifunctional aspartokinase/homoserine dehydrogenase.

Accordingly, in one embodiment, the process of the present invention for producing methionine in a non-human organism, like a microorganism or a plant or a part thereof, comprises increasing or generating the activity of a gene product with the activity of a gene product conferring the activity of “bifunctional aspartokinase/homoserine dehydrogenase”, especially from Arabidopsis thaliana or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said At1g31230, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said At1g31230, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said At1g31230, or a functional equivalent or a homolog thereof as depicted in column 8 of Table II, application no. 1, preferably a homolog or functional equivalent as depicted in column 8 of Table II B, application no. 1, and being depicted in the same respective line as said At1g31230, and preferably the activity is increased non-targeted.

Accordingly, in one embodiment, the molecule which activity is to be increased in the process of the invention is the gene product with an activity as a “bifunctional aspartokinase/homoserine dehydrogenase”, preferably it is the molecule of section (a) or (b) of this paragraph.

In particular, it was observed that in plants, especially in Arabidopsis thaliana, increasing or generating the activity of a gene product non-targeted with the activity of a “bifunctional aspartokinase/homoserine dehydrogenase”, preferably being encoded by a gene comprising the nucleic acid sequence SEQ ID NO. 627, preferably the coding region thereof, conferred the production of or the increase in methionine compared with the wild type control.

The nucleic acid sequence of At1g48040 from Arabidopsis thaliana, e.g. as shown in the respective line in column 5 of Table I, application no. 1 is unpublished. And the activity of the gene product thereof is the activity of protein phosphatase.

Accordingly, in one embodiment, the process of the present invention for producing methionine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said At1g48040, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said At1g48040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said At1g48040, or a functional equivalent or a homolog thereof as depicted in column 8 of Table II, application no. 1, preferably a homolog or functional equivalent as depicted in column 8 of Table II B, application no. 1, and being depicted in the same respective line as said At1g48040, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 812, preferably the coding region thereof, conferred the production of or the increase in methionine compared with the wild type control.

The nucleic acid sequence of At1g68320 from Arabidopsis thaliana, e.g. as shown in the respective line in column 5 of Table I, application no. 1 is unpublished. And the activity of the gene product thereof is the activity of transcription factor.

Accordingly, in one embodiment, the process of the present invention for producing methionine in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said At1g68320, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said At1g68320, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said At1g68320, or a functional equivalent or a homolog thereof as depicted in column 8 of Table II, application no. 1, preferably a homolog or functional equivalent as depicted in column 8 of Table II B, application no. 1, and being depicted in the same respective line as said At1g68320, and preferably the activity is increased non-targeted.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 1061, preferably the coding region thereof, conferred the production of or the increase in methionine compared with the wild type control.

The nucleic acid sequence of At2g25070 from Arabidopsis thaliana, e.g. as shown in the respective line in column 5 of Table I, application no. 1 is unpublished. And the activity of the gene product thereof is the activity of protein phosphatase.

Accordingly, in one embodiment, the process of the present invention for producing methionine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said

At2g25070, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said At2g25070, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said At2g25070, or a functional equivalent or a homolog thereof as depicted in column 8 of Table II, application no. 1, preferably a homolog or functional equivalent as depicted in column 8 of Table II B, application no. 1, and being depicted in the same respective line as said At2g25070, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 1298, preferably the coding region thereof, conferred the production of or the increase in methionine compared with the wild type control.

The nucleic acid sequence of At2g45420 from Arabidopsis thaliana, e.g. as shown in the respective line in column 5 of Table I, application no. 1 is unpublished. And the activity of the gene product thereof is the activity of At2g45420-protein.

Accordingly, in one embodiment, the process of the present invention for producing methionine in a non-human organism, like a microorganism or a plant or a part thereof, comprises increasing or generating the activity of a gene product with the activity of a gene product conferring the activity of “At2g45420-protein”, especially from Arabidopsis thaliana or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said At2g45420, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said At2g45420, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said At2g45420, or a functional equivalent or a homolog thereof as depicted in column 8 of Table II, application no. 1, preferably a homolog or functional equivalent as depicted in column 8 of Table II B, application no. 1, and being depicted in the same respective line as said At2g45420, and preferably the activity is increased non-targeted.

Accordingly, in one embodiment, the molecule which activity is to be increased in the process of the invention is the gene product with an activity as a “At2g45420-protein”, preferably it is the molecule of section (a) or (b) of this paragraph.

In particular, it was observed that in plants, especially in Arabidopsis thaliana, increasing or generating the activity of a gene product non-targeted with the activity of a “At2g45420-protein”, preferably being encoded by a gene comprising the nucleic acid sequence SEQ ID NO. 1623, preferably the coding region thereof, conferred the production of or the increase in methionine compared with the wild type control.

The nucleic acid sequence of At3g09820 from Arabidopsis thaliana, e.g. as shown in the respective line in column 5 of Table I, application no. 1 is unpublished. And the activity of the gene product thereof is the activity of adenosine kinase.

Accordingly, in one embodiment, the process of the present invention for producing methionine in a non-human organism, like a microorganism or a plant or a part thereof, comprises increasing or generating the activity of a gene product with the activity of a gene product conferring the activity of “adenosine kinase”, especially from Arabidopsis thaliana or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said At3g09820, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said At3g09820, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said At3g09820, or a functional equivalent or a homolog thereof as depicted in column 8 of Table II, application no. 1, preferably a homolog or functional equivalent as depicted in column 8 of Table II B, application no. 1, and being depicted in the same respective line as said At3g09820, and preferably the activity is increased non-targeted.

Accordingly, in one embodiment, the molecule which activity is to be increased in the process of the invention is the gene product with an activity as a “adenosine kinase”, preferably it is the molecule of section (a) or (b) of this paragraph.

In particular, it was observed that in plants, especially in Arabidopsis thaliana, increasing or generating the activity of a gene product non-targeted with the activity of a “adenosine kinase”, preferably being encoded by a gene comprising the nucleic acid sequence SEQ ID NO. 1696, preferably the coding region thereof, conferred the production of or the increase in methionine compared with the wild type control.

The nucleic acid sequence of At3g23000 from Arabidopsis thaliana, e.g. as shown in the respective line in column 5 of Table I, application no. 1 is unpublished. And the activity of the gene product thereof is the activity of protein kinase.

Accordingly, in one embodiment, the process of the present invention for producing methionine in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said At3g23000, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said At3g23000, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said At3g23000, or a functional equivalent or a homolog thereof as depicted in column 8 of Table II, application no. 1, preferably a homolog or functional equivalent as depicted in column 8 of Table II B, application no. 1, and being depicted in the same respective line as said At3g23000, and preferably the activity is increased non-targeted.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 1815, preferably the coding region thereof, conferred the production of or the increase in methionine compared with the wild type control.

The nucleic acid sequence of At3g61830 from Arabidopsis thaliana, e.g. as shown in the respective line in column 5 of Table I, application no. 1, is unpublished. And the activity of the gene product thereof is the activity of auxin response factor.

Accordingly, in one embodiment, the process of the present invention for producing methionine in a non-human organism, like a microorganism or a plant or a part thereof, comprises increasing or generating the activity of a gene product with the activity of a gene product conferring the activity of “auxin response factor”, especially from Arabidopsis thaliana or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said At3g61830, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said At3g61830, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said At3g61830, or a functional equivalent or a homolog thereof as depicted in column 8 of Table II, application no. 1, preferably a homolog or functional equivalent as depicted in column 8 of Table II B, application no. 1, and being depicted in the same respective line as said At3g61830, and preferably the activity is increased non-targeted.

Accordingly, in one embodiment, the molecule which activity is to be increased in the process of the invention is the gene product with an activity as a “auxin response factor”, preferably it is the molecule of section (a) or (b) of this paragraph.

In particular, it was observed that in plants, especially in Arabidopsis thaliana, increasing or generating the activity of a gene product non-targeted with the activity of a “auxin response factor”, preferably being encoded by a gene comprising the nucleic acid sequence SEQ ID NO. 2367, preferably the coding region thereof, conferred the production of or the increase in methionine compared with the wild type control.

The nucleic acid sequence of At3g62930 from Arabidopsis thaliana, e.g. as shown in the respective line in column 5 of Table I, application no. 1 is unpublished. And the activity of the gene product thereof is the activity of monthiol glutaredoxin.

Accordingly, in one embodiment, the process of the present invention for producing methionine in a non-human organism, like a microorganism or a plant or a part thereof, comprises increasing or generating the activity of a gene product with the activity of a gene product conferring the activity of “monthiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said At3g62930, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said At3g62930, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said At3g62930, or a functional equivalent or a homolog thereof as depicted in column 8 of Table II, application no. 1, preferably a homolog or functional equivalent as depicted in column 8 of Table II B, application no. 1, and being depicted in the same respective line as said At3g62930, and preferably the activity is increased non-targeted.

Accordingly, in one embodiment, the molecule which activity is to be increased in the process of the invention is the gene product with an activity as a “monthiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monthiol glutaredoxin”, preferably being encoded by a gene comprising the nucleic acid sequence SEQ ID NO. 2573, preferably the coding region thereof, conferred the production of or the increase in methionine compared with the wild type control.

The nucleic acid sequence of At3g62950 from Arabidopsis thaliana, e.g. as shown in the respective line in column 5 of Table I, application no. 1 is unpublished. And the activity of the gene product thereof is the activity of glutaredoxin.

Accordingly, in one embodiment, the process of the present invention for producing methionine in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said At3g62950, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said At3g62950, or a functional equivalent or a homolog thereof as depicted in column 8 of Table II, application no. 1, preferably a homolog or functional equivalent as depicted in column 8 of Table II B, application no. 1, and being depicted in the same respective line as said At3g62950, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in methionine compared with the wild type control.

The nucleic acid sequence of At4g15670 from Arabidopsis thaliana, e.g. as shown in the respective line in column 5 of Table I, application no. 1 is unpublished. And the activity of the gene product thereof is the activity of monothiol glutaredoxin.

Accordingly, in one embodiment, the process of the present invention for producing methionine in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said At4g15670, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said At4g15670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said At4g15670, or a functional equivalent or a homolog thereof as depicted in column 8 of Table II, application no. 1, preferably a homolog or functional equivalent as depicted in column 8 of Table II B, application no. 1, and being depicted in the same respective line as said At4g15670, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3279, preferably the coding region thereof, conferred the production of or the increase in methionine compared with the wild type control.

The nucleic acid sequence of At4g15700 from Arabidopsis thaliana, e.g. as shown in the respective line in column 5 of Table I, application no. 1 is unpublished. And the activity of the gene product thereof is the activity of monothiol glutaredoxin.

Accordingly, in one embodiment, the process of the present invention for producing methionine in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said At4g15700, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said At4g15700, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said At4g15700, or a functional equivalent or a homolog thereof as depicted in column 8 of Table II, application no. 1, preferably a homolog or functional equivalent as depicted in column 8 of Table II B, application no. 1, and being depicted in the same respective line as said At4g15700, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3654, preferably the coding region thereof, conferred the production of or the increase in methionine compared with the wild type control.

The nucleic acid sequence of At4g32480 from Arabidopsis thaliana, e.g. as shown in the respective line in column 5 of Table I, application no. 1 is unpublished. And the activity of the gene product thereof is the activity of At4g32480-protein.

Accordingly, in one embodiment, the process of the present invention for producing methionine in a non-human organism, like a microorganism or a plant or a part thereof, comprises increasing or generating the activity of a gene product with the activity of a gene product conferring the activity of “At4g32480-protein”, especially from Arabidopsis thaliana or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said At4g32480, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said At4g32480, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said At4g32480, or a functional equivalent or a homolog thereof as depicted in column 8 of Table II, application no. 1, preferably a homolog or functional equivalent as depicted in column 8 of Table II B, application no. 1, and being depicted in the same respective line as said At4g32480, and preferably the activity is increased non-targeted.

Accordingly, in one embodiment, the molecule which activity is to be increased in the process of the invention is the gene product with an activity as a “At4g32480-protein”, preferably it is the molecule of section (a) or (b) of this paragraph.

In particular, it was observed that in plants, especially in Arabidopsis thaliana, increasing or generating the activity of a gene product non-targeted with the activity of a “At4g32480-protein”, preferably being encoded by a gene comprising the nucleic acid sequence SEQ ID NO. 4040, preferably the coding region thereof, conferred the production of or the increase in methionine compared with the wild type control.

The nucleic acid sequence of At4g33040 from Arabidopsis thaliana, e.g. as shown in the respective line in column 5 of Table I, application no. 1 is unpublished. And the activity of the gene product thereof is the activity of glutaredoxin.

Accordingly, in one embodiment, the process of the present invention for producing methionine in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said

At4g33040, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said At4g33040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said At4g33040, or a functional equivalent or a homolog thereof as depicted in column 8 of Table II, application no. 1, preferably a homolog or functional equivalent as depicted in column 8 of Table II B, application no. 1, and being depicted in the same respective line as said At4g33040, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 4102, preferably the coding region thereof, conferred the production of or the increase in methionine compared with the wild type control.

The nucleic acid sequence of At4g35310 from Arabidopsis thaliana, e.g. as shown in the respective line in column 5 of Table I, application no. 1 is unpublished. And the activity of the gene product thereof is the activity of calcium-dependent protein kinase.

Accordingly, in one embodiment, the process of the present invention for producing methionine in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said At4g35310, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said At4g35310, or a functional equivalent or a homolog thereof as depicted in column 8 of Table II, application no. 1, preferably a homolog or functional equivalent as depicted in column 8 of Table II B, application no. 1, and being depicted in the same respective line as said At4g35310, and preferably the activity is increased non-targeted.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in methionine compared with the wild type control.

The nucleic acid sequence of At5g18600 from Arabidopsis thaliana, e.g. as shown in the respective line in column 5 of Table I, application no. 1 is unpublished. And the activity of the gene product thereof is the activity of monothiol glutaredoxin.

Accordingly, in one embodiment, the process of the present invention for producing methionine in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said At5g18600, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said At5g18600, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said At5g18600, or a functional equivalent or a homolog thereof as depicted in column 8 of Table II, application no. 1, preferably a homolog or functional equivalent as depicted in column 8 of Table II B, application no. 1, and being depicted in the same respective line as said At5g18600, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 4904, preferably the coding region thereof, conferred the production of or the increase in methionine compared with the wild type control.

The nucleic acid sequence of At5g57050 from Arabidopsis thaliana, e.g. as shown in the respective line in column 5 of Table I, application no. 1 is unpublished. And the activity of the gene product thereof is the activity of protein phosphatase.

Accordingly, in one embodiment, the process of the present invention for producing methionine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said At5g57050, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said At5g57050, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said At5g57050, or a functional equivalent or a homolog thereof as depicted in column 8 of Table II, application no. 1, preferably a homolog or functional equivalent as depicted in column 8 of Table II B, application no. 1, and being depicted in the same respective line as said At5g57050, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 5318, preferably the coding region thereof, conferred the production of or the increase in methionine compared with the wild type control.

The nucleic acid sequence of At5g64920 from Arabidopsis thaliana, e.g. as shown in the respective line in column 5 of Table I, application no. 1 is unpublished. And the activity of the gene product thereof is the activity of zinc finger protein.

Accordingly, in one embodiment, the process of the present invention for producing methionine in a non-human organism, like a microorganism or a plant or a part thereof, comprises increasing or generating the activity of a gene product with the activity of a gene product conferring the activity of “zinc finger protein”, especially from Arabidopsis thaliana or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said At5g64920, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said At5g64920, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said At5g64920, or a functional equivalent or a homolog thereof as depicted in column 8 of Table II, application no. 1, preferably a homolog or functional equivalent as depicted in column 8 of Table II B, application no. 1, and being depicted in the same respective line as said At5g64920, and preferably the activity is increased non-targeted.

Accordingly, in one embodiment, the molecule which activity is to be increased in the process of the invention is the gene product with an activity as a “zinc finger protein”, preferably it is the molecule of section (a) or (b) of this paragraph. In particular, it was observed that in plants, especially in Arabidopsis thaliana, increasing or generating the activity of a gene product non-targeted with the activity of a “zinc finger protein”, preferably being encoded by a gene comprising the nucleic acid sequence SEQ ID NO. 5493, preferably the coding region thereof, conferred the production of or the increase in methionine compared with the wild type control.

The nucleic acid sequence of AvinDRAFT_{—}1495 from Azotobacter vinelandii, e.g. as shown in the respective line in column 5 of Table I, application no. 1, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of malic enzyme.

Accordingly, in one embodiment, the process of the present invention for producing methionine in a non-human organism, like a microorganism or a plant or a part thereof, comprises increasing or generating the activity of a gene product with the activity of a gene product conferring the activity of “malic enzyme”, especially from Azotobacter vinelandii or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said AvinDRAFT_{—}1495, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said AvinDRAFT_{—}1495, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said AvinDRAFT _{—}1495, or a functional equivalent or a homolog thereof as depicted in column 8 of Table II, application no. 1, preferably a homolog or functional equivalent as depicted in column 8 of Table II B, application no. 1, and being depicted in the same respective line as said AvinDRAFT_{—}1495, and preferably the activity is increased non-targeted.

Accordingly, in one embodiment, the molecule which activity is to be increased in the process of the invention is the gene product with an activity as a “malic enzyme”, preferably it is the molecule of section (a) or (b) of this paragraph.

In particular, it was observed that in plants, especially in Arabidopsis thaliana, increasing or generating the activity of a gene product non-targeted with the activity of a “malic enzyme”, preferably being encoded by a gene comprising the nucleic acid sequence SEQ ID NO. 5557, preferably the coding region thereof, conferred the production of or the increase in methionine compared with the wild type control.

The nucleic acid sequence of AvinDRAFT_{—}2091 from Azotobacter vinelandii, e.g. as shown in the respective line in column 5 of Table I, application no. 1, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of beta-hydroxylase.

Accordingly, in one embodiment, the process of the present invention for producing methionine in a non-human organism, like a microorganism or a plant or a part thereof, comprises increasing or generating the activity of a gene product with the activity of a gene product conferring the activity of “beta-hydroxylase”, especially from Azotobacter vinelandii or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said AvinDRAFT_{—}2091, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said AvinDRAFT_{—}2091, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said AvinDRAFT_{—}2091, or a functional equivalent or a homolog thereof as depicted in column 8 of Table II, application no. 1, preferably a homolog or functional equivalent as depicted in column 8 of Table II B, application no. 1, and being depicted in the same respective line as said AvinDRAFT_{—}2091, and preferably the activity is increased non-targeted.

Accordingly, in one embodiment, the molecule which activity is to be increased in the process of the invention is the gene product with an activity as a “beta-hydroxylase”, preferably it is the molecule of section (a) or (b) of this paragraph.

In particular, it was observed that in plants, especially in Arabidopsis thaliana, increasing or generating the activity of a gene product non-targeted with the activity of a “beta-hydroxylase”, preferably being encoded by a gene comprising the nucleic acid sequence SEQ ID NO. 6040, preferably the coding region thereof, conferred the production of or the increase in methionine compared with the wild type control.

The nucleic acid sequence of AvinDRAFT_{—}3028 from Azotobacter vinelandii, e.g. as shown in the respective line in column 5 of Table I, application no. 1, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of 2-oxoglutarate dehydrogenase E1 subunit.

Accordingly, in one embodiment, the process of the present invention for producing methionine in a non-human organism, like a microorganism or a plant or a part thereof, comprises increasing or generating the activity of a gene product with the activity of a gene product conferring the activity of “2-oxoglutarate dehydrogenase E1 subunit”, especially from Azotobacter vinelandii or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said AvinDRAFT_{—}3028, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said AvinDRAFT_{—}3028, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said AvinDRAFT_{—}3028, or a functional equivalent or a homolog thereof as depicted in column 8 of Table II, application no. 1, preferably a homolog or functional equivalent as depicted in column 8 of Table II B, application no. 1, and being depicted in the same respective line as said AvinDRAFT_{—}3028, and preferably the activity is increased non-targeted.

Accordingly, in one embodiment, the molecule which activity is to be increased in the process of the invention is the gene product with an activity as a “2-oxoglutarate dehydrogenase E1 subunit”, preferably it is the molecule of section (a) or (b) of this paragraph.

In particular, it was observed that in plants, especially in Arabidopsis thaliana, increasing or generating the activity of a gene product non-targeted with the activity of a “2-oxoglutarate dehydrogenase E1 subunit”, preferably being encoded by a gene comprising the nucleic acid sequence SEQ ID NO. 6075, preferably the coding region thereof, conferred the production of or the increase in methionine compared with the wild type control.

The nucleic acid sequence of AvinDRAFT_{—}3546 from Azotobacter vinelandii, e.g. as shown in the respective line in column 5 of Table I, application no. 1, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of homoserine dehydrogenase.

Accordingly, in one embodiment, the process of the present invention for producing methionine in a non-human organism, like a microorganism or a plant or a part thereof, comprises increasing or generating the activity of a gene product with the activity of a gene product conferring the activity of “homoserine dehydrogenase”, especially from Azotobacter vinelandii or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said AvinDRAFT_{—}3546, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said AvinDRAFT_{—}3546, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said AvinDRAFT_{—}3546, or a functional equivalent or a homolog thereof as depicted in column 8 of Table II, application no. 1, preferably a homolog or functional equivalent as depicted in column 8 of Table II B, application no. 1, and being depicted in the same respective line as said AvinDRAFT_{—}3546, and preferably the activity is increased non-targeted.

Accordingly, in one embodiment, the molecule which activity is to be increased in the process of the invention is the gene product with an activity as a “homoserine dehydrogenase”, preferably it is the molecule of section (a) or (b) of this paragraph.

In particular, it was observed that in plants, especially in Arabidopsis thaliana, increasing or generating the activity of a gene product non-targeted with the activity of a “homoserine dehydrogenase”, preferably being encoded by a gene comprising the nucleic acid sequence SEQ ID NO. 6268, preferably the coding region thereof, conferred the production of or the increase in methionine compared with the wild type control.

The nucleic acid sequence of AvinDRAFT_{—}5103 from Azotobacter vinelandii, e.g. as shown in the respective line in column 5 of Table I, application no. 1, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of hydrolase.

Accordingly, in one embodiment, the process of the present invention for producing methionine in a non-human organism, like a microorganism or a plant or a part thereof, comprises increasing or generating the activity of a gene product with the activity of a gene product conferring the activity of “hydrolase”, especially from Azotobacter vinelandii or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said AvinDRAFT_{—}5103, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said AvinDRAFT_{—}5103, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said AvinDRAFT_{—}5103, or a functional equivalent or a homolog thereof as depicted in column 8 of Table II, application no. 1, preferably a homolog or functional equivalent as depicted in column 8 of Table II B, application no. 1, and being depicted in the same respective line as said AvinDRAFT_{—}5103, and preferably the activity is increased non-targeted.

Accordingly, in one embodiment, the molecule which activity is to be increased in the process of the invention is the gene product with an activity as a “hydrolase”, preferably it is the molecule of section (a) or (b) of this paragraph.

In particular, it was observed that in plants, especially in Arabidopsis thaliana, increasing or generating the activity of a gene product non-targeted with the activity of a “hydrolase”, preferably being encoded by a gene comprising the nucleic acid sequence SEQ ID NO. 6510, preferably the coding region thereof, conferred the production of or the increase in methionine compared with the wild type control.

The nucleic acid sequence of AvinDRAFT_{—}6075 from Azotobacter vinelandii, e.g. as shown in the respective line in column 5 of Table I, application no. 1, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of peptidyl-prolyl cis-trans isomerase.

Accordingly, in one embodiment, the process of the present invention for producing methionine in a non-human organism, like a microorganism or a plant or a part thereof, comprises increasing or generating the activity of a gene product with the activity of a gene product conferring the activity of “peptidyl-prolyl cis-trans isomerase”, especially from Azotobacter vinelandii or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said AvinDRAFT_{—}6075, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said AvinDRAFT_{—}6075, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said AvinDRAFT_{—}6075, or a functional equivalent or a homolog thereof as depicted in column 8 of Table II, application no. 1, preferably a homolog or functional equivalent as depicted in column 8 of Table II B, application no. 1, and being depicted in the same respective line as said AvinDRAFT_{—}6075, and preferably the activity is increased non-targeted.

Accordingly, in one embodiment, the molecule which activity is to be increased in the process of the invention is the gene product with an activity as a “peptidyl-prolyl cis-trans isomerase”, preferably it is the molecule of section (a) or (b) of this paragraph.

In particular, it was observed that in plants, especially in Arabidopsis thaliana, increasing or generating the activity of a gene product non-targeted with the activity of a “peptidyl-prolyl cis-trans isomerase”, preferably being encoded by a gene comprising the nucleic acid sequence SEQ ID NO. 6674, preferably the coding region thereof, conferred the production of or the increase in methionine compared with the wild type control.

The nucleic acid sequence of B0012 from Escherichia coli, e.g. as shown in the respective line in column 5 of Table I, application no. 1, has been published in Blattner et al., Science 277 (5331), 1453 (1997). And the activity of the gene product thereof is the activity of b0012-protein.

Accordingly, in one embodiment, the process of the present invention for producing methionine in a non-human organism, like a microorganism or a plant or a part thereof, comprises increasing or generating the activity of a gene product with the activity of a gene product conferring the activity of “b0012-protein”, especially from Escherichia coli or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said B0012, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said B0012, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said B0012, or a functional equivalent or a homolog thereof as depicted in column 8 of Table II, application no. 1, preferably a homolog or functional equivalent as depicted in column 8 of Table II B, application no. 1, and being depicted in the same respective line as said B0012, and preferably the activity is increased non-targeted.

Accordingly, in one embodiment, the molecule which activity is to be increased in the process of the invention is the gene product with an activity as a “b0012-protein”, preferably it is the molecule of section (a) or (b) of this paragraph.

In particular, it was observed that in plants, especially in Arabidopsis thaliana, increasing or generating the activity of a gene product non-targeted with the activity of a “b0012-protein”, preferably being encoded by a gene comprising the nucleic acid sequence SEQ ID NO. 6810, preferably the coding region thereof, conferred the production of or the increase in methionine compared with the wild type control.

The nucleic acid sequence of B0078 from Escherichia coli, e.g. as shown in the respective line in column 5 of Table I, application no. 1, has been published in Blattner et al., Science 277 (5331), 1453 (1997). And the activity of the gene product thereof is the activity of acetolactate synthase small subunit.

Accordingly, in one embodiment, the process of the present invention for producing methionine in a non-human organism, like a microorganism or a plant or a part thereof, comprises increasing or generating the activity of a gene product with the activity of a gene product conferring the activity of “acetolactate synthase small subunit”, especially from Escherichia coli or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said B0078, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said B0078, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said B0078, or a functional equivalent or a homolog thereof as depicted in column 8 of Table II, application no. 1, preferably a homolog or functional equivalent as depicted in column 8 of Table II B, application no. 1, and being depicted in the same respective line as said B0078, and preferably the activity is increased non-targeted.

Accordingly, in one embodiment, the molecule which activity is to be increased in the process of the invention is the gene product with an activity as a “acetolactate synthase small subunit”, preferably it is the molecule of section (a) or (b) of this paragraph.

In particular, it was observed that in plants, especially in Arabidopsis thaliana, increasing or generating the activity of a gene product non-targeted with the activity of a “acetolactate synthase small subunit”, preferably being encoded by a gene comprising the nucleic acid sequence SEQ ID NO. 6818, preferably the coding region thereof, conferred the production of or the increase in methionine compared with the wild type control.

The nucleic acid sequence of B0161 from Escherichia coli, e.g. as shown in the respective line in column 5 of Table I, application no. 1, has been published in Blattner et al., Science 277 (5331), 1453 (1997). And the activity of the gene product thereof is the activity of serine protease.

Accordingly, in one embodiment, the process of the present invention for producing methionine in a non-human organism, like a microorganism or a plant or a part thereof, comprises increasing or generating the activity of a gene product with the activity of a gene product conferring the activity of “serine protease”, especially from Escherichia coli or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said B0161, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said B0161, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said B0161, or a functional equivalent or a homolog thereof as depicted in column 8 of Table II, application no. 1, preferably a homolog or functional equivalent as depicted in column 8 of Table II B, application no. 1, and being depicted in the same respective line as said B0161, and preferably the activity is increased non-targeted.

Accordingly, in one embodiment, the molecule which activity is to be increased in the process of the invention is the gene product with an activity as a “serine protease”, preferably it is the molecule of section (a) or (b) of this paragraph.

In particular, it was observed that in plants, especially in Arabidopsis thaliana, increasing or generating the activity of a gene product non-targeted with the activity of a “serine protease”, preferably being encoded by a gene comprising the nucleic acid sequence SEQ ID NO. 7081, preferably the coding region thereof, conferred the production of or the increase in methionine compared with the wild type control.

The nucleic acid sequence of B0348 from Escherichia coli, e.g. as shown in the respective line in column 5 of Table I, application no. 1, has been published in Blattner et al., Science 277 (5331), 1453 (1997). And the activity of the gene product thereof is the activity of 2,3-dihydroxyphenylpropionate 1,2-dioxygenase.

Accordingly, in one embodiment, the process of the present invention for producing methionine in a non-human organism, like a microorganism or a plant or a part thereof, comprises increasing or generating the activity of a gene product with the activity of a gene product conferring the activity of “2,3-dihydroxyphenylpropionate 1,2-dioxygenase”, especially from Escherichia coli or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said B0348, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said B0348, and preferably the activity is increased plastidic, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said

B0348, or a functional equivalent or a homolog thereof as depicted in column 8 of Table II, application no. 1, preferably a homolog or functional equivalent as depicted in column 8 of Table II B, application no. 1, and being depicted in the same respective line as said B0348, and preferably the activity is increased plastidic.

Accordingly, in one embodiment, the molecule which activity is to be increased in the process of the invention is the gene product with an activity as a “2,3-dihydroxyphenylpropionate 1,2-dioxygenase”, preferably it is the molecule of section (a) or (b) of this paragraph.

In particular, it was observed that in plants, especially in Arabidopsis thaliana, increasing or generating the activity of a gene product plastidic with the activity of a “2,3-dihydroxyphenylpropionate 1,2-dioxygenase”, preferably being encoded by a gene comprising the nucleic acid sequence SEQ ID NO. 7269, preferably the coding region thereof, conferred the production of or the increase in methionine compared with the wild type control.

The nucleic acid sequence of B0449 from Escherichia coli, e.g. as shown in the respective line in column 5 of Table I, application no. 1, has been published in Blattner et al., Science 277 (5331), 1453 (1997). And the activity of the gene product thereof is the activity of ATP-binding component of a transport system.

Accordingly, in one embodiment, the process of the present invention for producing methionine in a non-human organism, like a microorganism or a plant or a part thereof, comprises increasing or generating the activity of a gene product with the activity of a gene product conferring the activity of “ATP-binding component of a transport system”, especially from Escherichia coli or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said B0449, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said B0449, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said B0449, or a functional equivalent or a homolog thereof as depicted in column 8 of Table II, application no. 1, preferably a homolog or functional equivalent as depicted in column 8 of Table II B, application no. 1, and being depicted in the same respective line as said B0449, and preferably the activity is increased non-targeted.

Accordingly, in one embodiment, the molecule which activity is to be increased in the process of the invention is the gene product with an activity as a “ATP-binding component of a transport system”, preferably it is the molecule of section (a) or (b) of this paragraph.

In particular, it was observed that in plants, especially in Arabidopsis thaliana, increasing or generating the activity of a gene product non-targeted with the activity of a “ATP-binding component of a transport system”, preferably being encoded by a gene comprising the nucleic acid sequence SEQ ID NO. 7333, preferably the coding region thereof, conferred the production of or the increase in methionine compared with the wild type control.

The nucleic acid sequence of B0486 from Escherichia coli, e.g. as shown in the respective line in column 5 of Table I, application no. 1, has been published in Blattner et al., Science 277 (5331), 1453 (1997). And the activity of the gene product thereof is the activity of membrane transport protein.

Accordingly, in one embodiment, the process of the present invention for producing methionine in a non-human organism, like a microorganism or a plant or a part thereof, comprises increasing or generating the activity of a gene product with the activity of a gene product conferring the activity of “membrane transport protein”, especially from Escherichia coli or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said B0486, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said B0486, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said B0486, or a functional equivalent or a homolog thereof as depicted in column 8 of Table II, application no. 1, preferably a homolog or functional equivalent as depicted in column 8 of Table II B, application no. 1, and being depicted in the same respective line as said B0486, and preferably the activity is increased non-targeted.

Accordingly, in one embodiment, the molecule which activity is to be increased in the process of the invention is the gene product with an activity as a “membrane transport protein”, preferably it is the molecule of section (a) or (b) of this paragraph.

In particular, it was observed that in plants, especially in Arabidopsis thaliana, increasing or generating the activity of a gene product non-targeted with the activity of a “membrane transport protein”, preferably being encoded by a gene comprising the nucleic acid sequence SEQ ID NO. 7686, preferably the coding region thereof, conferred the production of or the increase in methionine compared with the wild type control.

The nucleic acid sequence of B0870 from Escherichia coli, e.g. as shown in the respective line in column 5 of Table I, application no. 1, has been published in Blattner et al., Science 277 (5331), 1453 (1997). And the activity of the gene product thereof is the activity of threonine aldolase.

Accordingly, in one embodiment, the process of the present invention for producing methionine in a non-human organism, like a microorganism or a plant or a part thereof, comprises increasing or generating the activity of a gene product with the activity of a gene product conferring the activity of “threonine aldolase”, especially from Escherichia coli or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said B0870, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said B0870, and preferably the activity is increased plastidic, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said B0870, or a functional equivalent or a homolog thereof as depicted in column 8 of Table II, application no. 1, preferably a homolog or functional equivalent as depicted in column 8 of Table II B, application no. 1, and being depicted in the same respective line as said B0870, and preferably the activity is increased plastidic.

Accordingly, in one embodiment, the molecule which activity is to be increased in the process of the invention is the gene product with an activity as a “threonine aldolase”, preferably it is the molecule of section (a) or (b) of this paragraph.

In particular, it was observed that in plants, especially in Arabidopsis thaliana, increasing or generating the activity of a gene product plastidic with the activity of a “threonine aldolase”, preferably being encoded by a gene comprising the nucleic acid sequence SEQ ID NO. 7730, preferably the coding region thereof, conferred the production of or the increase in methionine compared with the wild type control.

The nucleic acid sequence of B0898 from Escherichia coli, e.g. as shown in the respective line in column 5 of Table I, application no. 1, has been published in Blattner et al., Science 277 (5331), 1453 (1997). And the activity of the gene product thereof is the activity of major facilitator superfamily transporter protein.

Accordingly, in one embodiment, the process of the present invention for producing methionine in a non-human organism, like a microorganism or a plant or a part thereof, comprises increasing or generating the activity of a gene product with the activity of a gene product conferring the activity of “major facilitator superfamily transporter protein”, especially from Escherichia coli or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said B0898, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said B0898, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said B0898, or a functional equivalent or a homolog thereof as depicted in column 8 of Table II, application no. 1, preferably a homolog or functional equivalent as depicted in column 8 of Table II B, application no. 1, and being depicted in the same respective line as said B0898, and preferably the activity is increased non-targeted.

Accordingly, in one embodiment, the molecule which activity is to be increased in the process of the invention is the gene product with an activity as a “major facilitator superfamily transporter protein”, preferably it is the molecule of section (a) or (b) of this paragraph.

In particular, it was observed that in plants, especially in Arabidopsis thaliana, increasing or generating the activity of a gene product non-targeted with the activity of a “major facilitator superfamily transporter protein”, preferably being encoded by a gene comprising the nucleic acid sequence SEQ ID NO. 7917, preferably the coding region thereof, conferred the production of or the increase in methionine compared with the wild type control.

The nucleic acid sequence of B1003 from Escherichia coli, e.g. as shown in the respective line in column 5 of Table I, application no. 1, has been published in Blattner et al., Science 277 (5331), 1453 (1997). And the activity of the gene product thereof is the activity of b1003-protein.

Accordingly, in one embodiment, the process of the present invention for producing methionine in a non-human organism, like a microorganism or a plant or a part thereof, comprises increasing or generating the activity of a gene product with the activity of a gene product conferring the activity of “b1003-protein”, especially from Escherichia coli or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said B1003, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said B1003, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said B1003, or a functional equivalent or a homolog thereof as depicted in column 8 of Table II, application no. 1, preferably a homolog or functional equivalent as depicted in column 8 of Table II B, application no. 1, and being depicted in the same respective line as said B1003, and preferably the activity is increased non-targeted.

Accordingly, in one embodiment, the molecule which activity is to be increased in the process of the invention is the gene product with an activity as a “b1003-protein”, preferably it is the molecule of section (a) or (b) of this paragraph.

In particular, it was observed that in plants, especially in Arabidopsis thaliana, increasing or generating the activity of a gene product non-targeted with the activity of a “b1003-protein”, preferably being encoded by a gene comprising the nucleic acid sequence SEQ ID NO. 7941, preferably the coding region thereof, conferred the production of or the increase in methionine compared with the wild type control.

The nucleic acid sequence of B1522 from Escherichia coli, e.g. as shown in the respective line in column 5 of Table I, application no. 1, has been published in Blattner et al., Science 277 (5331), 1453 (1997). And the activity of the gene product thereof is the activity of b1522-protein.

Accordingly, in one embodiment, the process of the present invention for producing methionine in a non-human organism, like a microorganism or a plant or a part thereof, comprises increasing or generating the activity of a gene product with the activity of a gene product conferring the activity of “b1522-protein”, especially from Escherichia coli or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said B1522, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said B1522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said B1522, or a functional equivalent or a homolog thereof as depicted in column 8 of Table II, application no. 1, preferably a homolog or functional equivalent as depicted in column 8 of Table II B, application no. 1, and being depicted in the same respective line as said B1522, and preferably the activity is increased non-targeted.

Accordingly, in one embodiment, the molecule which activity is to be increased in the process of the invention is the gene product with an activity as a “b1522-protein”, preferably it is the molecule of section (a) or (b) of this paragraph. In particular, it was observed that in plants, especially in Arabidopsis thaliana, increasing or generating the activity of a gene product non-targeted with the activity of a “b1522-protein”, preferably being encoded by a gene comprising the nucleic acid sequence SEQ ID NO. 7947, preferably the coding region thereof, conferred the production of or the increase in methionine compared with the wild type control.

The nucleic acid sequence of B1601 from Escherichia coli, e.g. as shown in the respective line in column 5 of Table I, application no. 1, has been published in Blattner et al., Science 277 (5331), 1453 (1997). And the activity of the gene product thereof is the activity of transport protein.

Accordingly, in one embodiment, the process of the present invention for producing methionine in a non-human organism, like a microorganism or a plant or a part thereof, comprises increasing or generating the activity of a gene product with the activity of a gene product conferring the activity of “transport protein”, especially from Escherichia coli or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said B1601, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said B1601, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said B1601, or a functional equivalent or a homolog thereof as depicted in column 8 of Table II, application no. 1, preferably a homolog or functional equivalent as depicted in column 8 of Table II B, application no. 1, and being depicted in the same respective line as said B1601, and preferably the activity is increased non-targeted.

Accordingly, in one embodiment, the molecule which activity is to be increased in the process of the invention is the gene product with an activity as a “transport protein”, preferably it is the molecule of section (a) or (b) of this paragraph.

In particular, it was observed that in plants, especially in Arabidopsis thaliana, increasing or generating the activity of a gene product non-targeted with the activity of a “transport protein”, preferably being encoded by a gene comprising the nucleic acid sequence SEQ ID NO. 7992, preferably the coding region thereof, conferred the production of or the increase in methionine compared with the wild type control.

The nucleic acid sequence of B1814 from Escherichia coli, e.g. as shown in the respective line in column 5 of Table I, application no. 1, has been published in Blattner et al., Science 277 (5331), 1453 (1997). And the activity of the gene product thereof is the activity of L-serine dehydratase.

Accordingly, in one embodiment, the process of the present invention for producing methionine in a non-human organism, like a microorganism or a plant or a part thereof, comprises increasing or generating the activity of a gene product with the activity of a gene product conferring the activity of “L-serine dehydratase”, especially from Escherichia coli or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said B1814, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said B1814, and preferably the activity is increased plastidic, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said B1814, or a functional equivalent or a homolog thereof as depicted in column 8 of Table II, application no. 1, preferably a homolog or functional equivalent as depicted in column 8 of Table II B, application no. 1, and being depicted in the same respective line as said B1814, and preferably the activity is increased plastidic.

Accordingly, in one embodiment, the molecule which activity is to be increased in the process of the invention is the gene product with an activity as a “L-serine dehydratase”, preferably it is the molecule of section (a) or (b) of this paragraph.

In particular, it was observed that in plants, especially in Arabidopsis thaliana, increasing or generating the activity of a gene product plastidic with the activity of a “L-serine dehydratase”, preferably being encoded by a gene comprising the nucleic acid sequence SEQ ID NO. 8033, preferably the coding region thereof, conferred the production of or the increase in methionine compared with the wild type control.

The nucleic acid sequence of B1838 from Escherichia coli, e.g. as shown in the respective line in column 5 of Table I, application no. 1, has been published in Blattner et al., Science 277 (5331), 1453 (1997). And the activity of the gene product thereof is the activity of serine/threonine-protein phosphatase.

Accordingly, in one embodiment, the process of the present invention for producing methionine in a non-human organism, like a microorganism or a plant or a part thereof, comprises increasing or generating the activity of a gene product with the activity of a gene product conferring the activity of “serine/threonine-protein phosphatase”, especially from Escherichia coli or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said B1838, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said B1838, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said B1838, or a functional equivalent or a homolog thereof as depicted in column 8 of Table II, application no. 1, preferably a homolog or functional equivalent as depicted in column 8 of Table II B, application no. 1, and being depicted in the same respective line as said B1838, and preferably the activity is increased non-targeted.

Accordingly, in one embodiment, the molecule which activity is to be increased in the process of the invention is the gene product with an activity as a “serine/threonine-protein phosphatase”, preferably it is the molecule of section (a) or (b) of this paragraph.

In particular, it was observed that in plants, especially in Arabidopsis thaliana, increasing or generating the activity of a gene product non-targeted with the activity of a “serine/threonine-protein phosphatase”, preferably being encoded by a gene comprising the nucleic acid sequence SEQ ID NO. 8316, preferably the coding region thereof, conferred the production of or the increase in methionine compared with the wild type control.

The nucleic acid sequence of B1854 from Escherichia coli, e.g. as shown in the respective line in column 5 of Table I, application no. 1, has been published in Blattner et al., Science 277 (5331), 1453 (1997). And the activity of the gene product thereof is the activity of pyruvate kinase.

Accordingly, in one embodiment, the process of the present invention for producing methionine in a non-human organism, like a microorganism or a plant or a part thereof, comprises increasing or generating the activity of a gene product with the activity of a gene product conferring the activity of “pyruvate kinase”, especially from Escherichia coli or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said B1854, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said B1854, and preferably the activity is increased plastidic, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said B1854, or a functional equivalent or a homolog thereof as depicted in column 8 of Table II, application no. 1, preferably a homolog or functional equivalent as depicted in column 8 of Table II B, application no. 1, and being depicted in the same respective line as said B1854, and preferably the activity is increased plastidic.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 8363, preferably the coding region thereof, conferred the production of or the increase in methionine compared with the wild type control.

The nucleic acid sequence of B2032 from Escherichia coli, e.g. as shown in the respective line in column 5 of Table I, application no. 1, has been published in Blattner et al., Science 277 (5331), 1453 (1997). And the activity of the gene product thereof is the activity of b2032-protein.

Accordingly, in one embodiment, the process of the present invention for producing methionine in a non-human organism, like a microorganism or a plant or a part thereof, comprises increasing or generating the activity of a gene product with the activity of a gene product conferring the activity of “b2032-protein”, especially from Escherichia coli or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said B2032, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said B2032, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said B2032, or a functional equivalent or a homolog thereof as depicted in column 8 of Table II, application no. 1, preferably a homolog or functional equivalent as depicted in column 8 of Table II B, application no. 1, and being depicted in the same respective line as said B2032, and preferably the activity is increased non-targeted.

Accordingly, in one embodiment, the molecule which activity is to be increased in the process of the invention is the gene product with an activity as a “b2032-protein”, preferably it is the molecule of section (a) or (b) of this paragraph.

In particular, it was observed that in plants, especially in Arabidopsis thaliana, increasing or generating the activity of a gene product non-targeted with the activity of a “b2032-protein”, preferably being encoded by a gene comprising the nucleic acid sequence SEQ ID NO. 8920, preferably the coding region thereof, conferred the production of or the increase in methionine compared with the wild type control.

The nucleic acid sequence of B2066 from Escherichia coli, e.g. as shown in the respective line in column 5 of Table I, application no. 1, has been published in Blattner et al., Science 277 (5331), 1453 (1997). And the activity of the gene product thereof is the activity of uridine/cytidine kinase.

Accordingly, in one embodiment, the process of the present invention for producing methionine in a non-human organism, like a microorganism or a plant or a part thereof, comprises increasing or generating the activity of a gene product with the activity of a gene product conferring the activity of “uridine/cytidine kinase”, especially from Escherichia coli or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said B2066, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said B2066, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said B2066, or a functional equivalent or a homolog thereof as depicted in column 8 of Table II, application no. 1, preferably a homolog or functional equivalent as depicted in column 8 of Table II B, application no. 1, and being depicted in the same respective line as said B2066, and preferably the activity is increased mitochondrial.

Accordingly, in one embodiment, the molecule which activity is to be increased in the process of the invention is the gene product with an activity as a “uridine/cytidine kinase”, preferably it is the molecule of section (a) or (b) of this paragraph.

In particular, it was observed that in plants, especially in Arabidopsis thaliana, increasing or generating the activity of a gene product mitochondrial with the activity of a “uridine/cytidine kinase”, preferably being encoded by a gene comprising the nucleic acid sequence SEQ ID NO. 8937, preferably the coding region thereof, conferred the production of or the increase in methionine compared with the wild type control.

The nucleic acid sequence of B2345 from Escherichia coli, e.g. as shown in the respective line in column 5 of Table I, application no. 1, has been published in Blattner et al., Science 277 (5331), 1453 (1997). And the activity of the gene product thereof is the activity of b2345-protein.

Accordingly, in one embodiment, the process of the present invention for producing methionine in a non-human organism, like a microorganism or a plant or a part thereof, comprises increasing or generating the activity of a gene product with the activity of a gene product conferring the activity of “b2345-protein”, especially from Escherichia coli or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said B2345, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said B2345, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said B2345, or a functional equivalent or a homolog thereof as depicted in column 8 of Table II, application no. 1, preferably a homolog or functional equivalent as depicted in column 8 of Table II B, application no. 1, and being depicted in the same respective line as said B2345, and preferably the activity is increased non-targeted.

Accordingly, in one embodiment, the molecule which activity is to be increased in the process of the invention is the gene product with an activity as a “b2345-protein”, preferably it is the molecule of section (a) or (b) of this paragraph.

In particular, it was observed that in plants, especially in Arabidopsis thaliana, increasing or generating the activity of a gene product non-targeted with the activity of a “b2345-protein”, preferably being encoded by a gene comprising the nucleic acid sequence SEQ ID NO. 9156, preferably the coding region thereof, conferred the production of or the increase in methionine compared with the wild type control.

The nucleic acid sequence of B2513 from Escherichia coli, e.g. as shown in the respective line in column 5 of Table I, application no. 1, has been published in Blattner et al., Science 277 (5331), 1453 (1997). And the activity of the gene product thereof is the activity of b2513-protein.

Accordingly, in one embodiment, the process of the present invention for producing methionine in a non-human organism, like a microorganism or a plant or a part thereof, comprises increasing or generating the activity of a gene product with the activity of a gene product conferring the activity of “b2513-protein”, especially from Escherichia coli or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said B2513, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said B2513, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said B2513, or a functional equivalent or a homolog thereof as depicted in column 8 of Table II, application no. 1, preferably a homolog or functional equivalent as depicted in column 8 of Table II B, application no. 1, and being depicted in the same respective line as said B2513, and preferably the activity is increased non-targeted.

Accordingly, in one embodiment, the molecule which activity is to be increased in the process of the invention is the gene product with an activity as a “b2513-protein”, preferably it is the molecule of section (a) or (b) of this paragraph.

In particular, it was observed that in plants, especially in Arabidopsis thaliana, increasing or generating the activity of a gene product non-targeted with the activity of a “b2513-protein”, preferably being encoded by a gene comprising the nucleic acid sequence SEQ ID NO. 9167, preferably the coding region thereof, conferred the production of or the increase in methionine compared with the wild type control.

The nucleic acid sequence of B2673 from Escherichia coli, e.g. as shown in the respective line in column 5 of Table I, application no. 1, has been published in Blattner et al., Science 277 (5331), 1453 (1997). And the activity of the gene product thereof is the activity of b2673-protein.

Accordingly, in one embodiment, the process of the present invention for producing methionine in a non-human organism, like a microorganism or a plant or a part thereof, comprises increasing or generating the activity of a gene product with the activity of a gene product conferring the activity of “b2673-protein”, especially from Escherichia coli or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said B2673, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said B2673, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said B2673, or a functional equivalent or a homolog thereof as depicted in column 8 of Table II, application no. 1, preferably a homolog or functional equivalent as depicted in column 8 of Table II B, application no. 1, and being depicted in the same respective line as said B2673, and preferably the activity is increased non-targeted.

Accordingly, in one embodiment, the molecule which activity is to be increased in the process of the invention is the gene product with an activity as a “b2673-protein”, preferably it is the molecule of section (a) or (b) of this paragraph.

In particular, it was observed that in plants, especially in Arabidopsis thaliana, increasing or generating the activity of a gene product non-targeted with the activity of a “b2673-protein”, preferably being encoded by a gene comprising the nucleic acid sequence SEQ ID NO. 9244, preferably the coding region thereof, conferred the production of or the increase in methionine compared with the wild type control.

The nucleic acid sequence of B2923 from Escherichia coli, e.g. as shown in the respective line in column 5 of Table I, application no. 1, has been published in Blattner et al., Science 277 (5331), 1453 (1997). And the activity of the gene product thereof is the activity of arginine exporter protein.

Accordingly, in one embodiment, the process of the present invention for producing methionine in a non-human organism, like a microorganism or a plant or a part thereof, comprises increasing or generating the activity of a gene product with the activity of a gene product conferring the activity of “arginine exporter protein”, especially from Escherichia coli or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said B2923, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said B2923, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said B2923, or a functional equivalent or a homolog thereof as depicted in column 8 of Table II, application no. 1, preferably a homolog or functional equivalent as depicted in column 8 of Table II B, application no. 1, and being depicted in the same respective line as said B2923, and preferably the activity is increased non-targeted.

Accordingly, in one embodiment, the molecule which activity is to be increased in the process of the invention is the gene product with an activity as a “arginine exporter protein”, preferably it is the molecule of section (a) or (b) of this paragraph.
In particular, it was observed that in plants, especially in Arabidopsis thaliana, increasing or generating the activity of a gene product non-targeted with the activity of a “arginine exporter protein”, preferably being encoded by a gene comprising the nucleic acid sequence SEQ ID NO. 9333, preferably the coding region thereof, conferred the production of or the increase in methionine compared with the wild type control.

The nucleic acid sequence of B3246 from Escherichia coli, e.g. as shown in the respective line in column 5 of Table I, application no. 1, has been published in Blattner et al., Science 277 (5331), 1453 (1997). And the activity of the gene product thereof is the activity of b3246-protein.

Accordingly, in one embodiment, the process of the present invention for producing methionine in a non-human organism, like a microorganism or a plant or a part thereof, comprises increasing or generating the activity of a gene product with the activity of a gene product conferring the activity of “b3246-protein”, especially from Escherichia coli or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said B3246, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said B3246, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said B3246, or a functional equivalent or a homolog thereof as depicted in column 8 of Table II, application no. 1, preferably a homolog or functional equivalent as depicted in column 8 of Table II B, application no. 1, and being depicted in the same respective line as said B3246, and preferably the activity is increased non-targeted.

Accordingly, in one embodiment, the molecule which activity is to be increased in the process of the invention is the gene product with an activity as a “b3246-protein”, preferably it is the molecule of section (a) or (b) of this paragraph.

In particular, it was observed that in plants, especially in Arabidopsis thaliana, increasing or generating the activity of a gene product non-targeted with the activity of a “b3246-protein”, preferably being encoded by a gene comprising the nucleic acid sequence SEQ ID NO. 9470, preferably the coding region thereof, conferred the production of or the increase in methionine compared with the wild type control.

The nucleic acid sequence of B3256 from Escherichia coli, e.g. as shown in the respective line in column 5 of Table I, application no. 1, has been published in Blattner et al., Science 277 (5331), 1453 (1997). And the activity of the gene product thereof is the activity of acetyl CoA carboxylase.

Accordingly, in one embodiment, the process of the present invention for producing methionine in a non-human organism, like a microorganism or a plant or a part thereof, comprises increasing or generating the activity of a gene product with the activity of a gene product conferring the activity of “acetyl CoA carboxylase”, especially from Escherichia coli or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said B3256, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said B3256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said B3256, or a functional equivalent or a homolog thereof as depicted in column 8 of Table II, application no. 1, preferably a homolog or functional equivalent as depicted in column 8 of Table II B, application no. 1, and being depicted in the same respective line as said B3256, and preferably the activity is increased non-targeted.

Accordingly, in one embodiment, the molecule which activity is to be increased in the process of the invention is the gene product with an activity as a “acetyl CoA carboxylase”, preferably it is the molecule of section (a) or (b) of this paragraph.

In particular, it was observed that in plants, especially in Arabidopsis thaliana, increasing or generating the activity of a gene product non-targeted with the activity of a “acetyl CoA carboxylase”, preferably being encoded by a gene comprising the nucleic acid sequence SEQ ID NO. 9492, preferably the coding region thereof, conferred the production of or the increase in methionine compared with the wild type control.

The nucleic acid sequence of B3346 from Escherichia coli, e.g. as shown in the respective line in column 5 of Table I, application no. 1, has been published in Blattner et al., Science 277 (5331), 1453 (1997). And the activity of the gene product thereof is the activity of b3346-protein.

Accordingly, in one embodiment, the process of the present invention for producing methionine in a non-human organism, like a microorganism or a plant or a part thereof, comprises increasing or generating the activity of a gene product with the activity of a gene product conferring the activity of “b3346-protein”, especially from Escherichia coli or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said B3346, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said B3346, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said B3346, or a functional equivalent or a homolog thereof as depicted in column 8 of Table II, application no. 1, preferably a homolog or functional equivalent as depicted in column 8 of Table II B, application no. 1, and being depicted in the same respective line as said B3346, and preferably the activity is increased non-targeted.

Accordingly, in one embodiment, the molecule which activity is to be increased in the process of the invention is the gene product with an activity as a “b3346-protein”, preferably it is the molecule of section (a) or (b) of this paragraph.

In particular, it was observed that in plants, especially in Arabidopsis thaliana, increasing or generating the activity of a gene product non-targeted with the activity of a “b3346-protein”, preferably being encoded by a gene comprising the nucleic acid sequence SEQ ID NO. 10104, preferably the coding region thereof, conferred the production of or the increase in methionine compared with the wild type control.

The nucleic acid sequence of B3572 from Escherichia coli, e.g. as shown in the respective line in column 5 of Table I, application no. 1, has been published in Blattner et al., Science 277 (5331), 1453 (1997). And the activity of the gene product thereof is the activity of valine-pyruvate transaminase.

Accordingly, in one embodiment, the process of the present invention for producing methionine in a non-human organism, like a microorganism or a plant or a part thereof, comprises increasing or generating the activity of a gene product with the activity of a gene product conferring the activity of “valine-pyruvate transaminase”, especially from Escherichia coli or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said B3572, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said B3572, and preferably the activity is increased plastidic, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said B3572, or a functional equivalent or a homolog thereof as depicted in column 8 of Table II, application no. 1, preferably a homolog or functional equivalent as depicted in column 8 of Table II B, application no. 1, and being depicted in the same respective line as said B3572, and preferably the activity is increased plastidic.

Accordingly, in one embodiment, the molecule which activity is to be increased in the process of the invention is the gene product with an activity as a “valine-pyruvate transaminase”, preferably it is the molecule of section (a) or (b) of this paragraph.

In particular, it was observed that in plants, especially in Arabidopsis thaliana, increasing or generating the activity of a gene product plastidic with the activity of a “valine-pyruvate transaminase”, preferably being encoded by a gene comprising the nucleic acid sequence SEQ ID NO. 10172, preferably the coding region thereof, conferred the production of or the increase in methionine compared with the wild type control.

The nucleic acid sequence of B3771 from Escherichia coli, e.g. as shown in the respective line in column 5 of Table I, application no. 1, has been published in Blattner et al., Science 277 (5331), 1453 (1997). And the activity of the gene product thereof is the activity of dihydroxyacid dehydratase.

Accordingly, in one embodiment, the process of the present invention for producing methionine in a non-human organism, like a microorganism or a plant or a part thereof, comprises increasing or generating the activity of a gene product with the activity of a gene product conferring the activity of “dihydroxyacid dehydratase”, especially from Escherichia coli or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said B3771, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said B3771, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said B3771, or a functional equivalent or a homolog thereof as depicted in column 8 of Table II, application no. 1, preferably a homolog or functional equivalent as depicted in column 8 of Table II B, application no. 1, and being depicted in the same respective line as said B3771, and preferably the activity is increased non-targeted.

Accordingly, in one embodiment, the molecule which activity is to be increased in the process of the invention is the gene product with an activity as a “dihydroxyacid dehydratase”, preferably it is the molecule of section (a) or (b) of this paragraph.

In particular, it was observed that in plants, especially in Arabidopsis thaliana, increasing or generating the activity of a gene product non-targeted with the activity of a “dihydroxyacid dehydratase”, preferably being encoded by a gene comprising the nucleic acid sequence SEQ ID NO. 10252, preferably the coding region thereof, conferred the production of or the increase in methionine compared with the wild type control.

The nucleic acid sequence of B3817 from Escherichia coli, e.g. as shown in the respective line in column 5 of Table I, application no. 1, has been published in Blattner et al., Science 277 (5331), 1453 (1997). And the activity of the gene product thereof is the activity of b3817-protein.

Accordingly, in one embodiment, the process of the present invention for producing methionine in a non-human organism, like a microorganism or a plant or a part thereof, comprises increasing or generating the activity of a gene product with the activity of a gene product conferring the activity of “b3817-protein”, especially from Escherichia coli or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said B3817, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said B3817, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said B3817, or a functional equivalent or a homolog thereof as depicted in column 8 of Table II, application no. 1, preferably a homolog or functional equivalent as depicted in column 8 of Table II B, application no. 1, and being depicted in the same respective line as said B3817, and preferably the activity is increased non-targeted.

Accordingly, in one embodiment, the molecule which activity is to be increased in the process of the invention is the gene product with an activity as a “b3817-protein”, preferably it is the molecule of section (a) or (b) of this paragraph.

In particular, it was observed that in plants, especially in Arabidopsis thaliana, increasing or generating the activity of a gene product non-targeted with the activity of a “b3817-protein”, preferably being encoded by a gene comprising the nucleic acid sequence SEQ ID NO. 10708, preferably the coding region thereof, conferred the production of or the increase in methionine compared with the wild type control.

The nucleic acid sequence of B3823 from Escherichia coli, e.g. as shown in the respective line in column 5 of Table I, application no. 1, has been published in Blattner et al., Science 277 (5331), 1453 (1997). And the activity of the gene product thereof is the activity of threonine efflux protein.

Accordingly, in one embodiment, the process of the present invention for producing methionine in a non-human organism, like a microorganism or a plant or a part thereof, comprises increasing or generating the activity of a gene product with the activity of a gene product conferring the activity of “threonine efflux protein”, especially from Escherichia coli or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said B3823, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said B3823, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said B3823, or a functional equivalent or a homolog thereof as depicted in column 8 of Table II, application no. 1, preferably a homolog or functional equivalent as depicted in column 8 of Table II B, application no. 1, and being depicted in the same respective line as said B3823, and preferably the activity is increased non-targeted.

Accordingly, in one embodiment, the molecule which activity is to be increased in the process of the invention is the gene product with an activity as a “threonine efflux protein”, preferably it is the molecule of section (a) or (b) of this paragraph.

In particular, it was observed that in plants, especially in Arabidopsis thaliana, increasing or generating the activity of a gene product non-targeted with the activity of a “threonine efflux protein”, preferably being encoded by a gene comprising the nucleic acid sequence SEQ ID NO. 10726, preferably the coding region thereof, conferred the production of or the increase in methionine compared with the wild type control.

The nucleic acid sequence of B4029 from Escherichia coli, e.g. as shown in the respective line in column 5 of Table I, application no. 1, has been published in Blattner et al., Science 277 (5331), 1453 (1997). And the activity of the gene product thereof is the activity of b4029-protein.

Accordingly, in one embodiment, the process of the present invention for producing methionine in a non-human organism, like a microorganism or a plant or a part thereof, comprises increasing or generating the activity of a gene product with the activity of a gene product conferring the activity of “b4029-protein”, especially from Escherichia coli or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said B4029, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said B4029, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said B4029, or a functional equivalent or a homolog thereof as depicted in column 8 of Table II, application no. 1, preferably a homolog or functional equivalent as depicted in column 8 of Table II B, application no. 1, and being depicted in the same respective line as said B4029, and preferably the activity is increased non-targeted.

Accordingly, in one embodiment, the molecule which activity is to be increased in the process of the invention is the gene product with an activity as a “b4029-protein”, preferably it is the molecule of section (a) or (b) of this paragraph.

In particular, it was observed that in plants, especially in Arabidopsis thaliana, increasing or generating the activity of a gene product non-targeted with the activity of a “b4029-protein”, preferably being encoded by a gene comprising the nucleic acid sequence SEQ ID NO. 10740, preferably the coding region thereof, conferred the production of or the increase in methionine compared with the wild type control.

The nucleic acid sequence of GM02LC12622 from Glycine max, e.g. as shown in the respective line in column 5 of Table I, application no. 1 is unpublished. And the activity of the gene product thereof is the activity of glutaredoxin.

Accordingly, in one embodiment, the process of the present invention for producing methionine in a non-human organism, like a microorganism or a plant or a part thereof, comprises increasing or generating the activity of a gene product with the activity of a gene product conferring the activity of “glutaredoxin”, especially from Glycine max or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said GM02LC12622, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said GM02LC12622, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said GM02LC12622, or a functional equivalent or a homolog thereof as depicted in column 8 of Table II, application no. 1, preferably a homolog or functional equivalent as depicted in column 8 of Table II B, application no. 1, and being depicted in the same respective line as said GM02LC12622, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 10811, preferably the coding region thereof, conferred the production of or the increase in methionine compared with the wild type control.

The nucleic acid sequence of Sll1108 from Synechocystis sp., e.g. as shown in the respective line in column 5 of Table I, application no. 1, has been published in Nakamura et al., Nucleic Acids Res. 28(1), 72 (2000). And the activity of the gene product thereof is the activity of 5′-nucleotidase.

Accordingly, in one embodiment, the process of the present invention for producing methionine in a non-human organism, like a microorganism or a plant or a part thereof, comprises increasing or generating the activity of a gene product with the activity of a gene product conferring the activity of “5′-nucleotidase”, especially from Synechocystis sp. or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said Sll1108, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said Sll1108, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said Sll1108, or a functional equivalent or a homolog thereof as depicted in column 8 of Table II, application no. 1, preferably a homolog or functional equivalent as depicted in column 8 of Table II B, application no. 1, and being depicted in the same respective line as said Sll1108, and preferably the activity is increased mitochondrial.

Accordingly, in one embodiment, the molecule which activity is to be increased in the process of the invention is the gene product with an activity as a “5′-nucleotidase”, preferably it is the molecule of section (a) or (b) of this paragraph.

In particular, it was observed that in plants, especially in Arabidopsis thaliana, increasing or generating the activity of a gene product mitochondrial with the activity of a “5′-nucleotidase”, preferably being encoded by a gene comprising the nucleic acid sequence SEQ ID NO. 11211, preferably the coding region thereof, conferred the production of or the increase in methionine compared with the wild type control.

The nucleic acid sequence of Sll1545 from Synechocystis sp., e.g. as shown in the respective line in column 5 of Table I, application no. 1, has been published in Nakamura et al., Nucleic Acids Res. 28(1), 72 (2000). And the activity of the gene product thereof is the activity of glutathione S-transferase.

Accordingly, in one embodiment, the process of the present invention for producing methionine in a non-human organism, like a microorganism or a plant or a part thereof, comprises increasing or generating the activity of a gene product with the activity of a gene product conferring the activity of “glutathione S-transferase”, especially from Synechocystis sp. or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said Sll1545, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said Sll1545, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said Sll1545, or a functional equivalent or a homolog thereof as depicted in column 8 of Table II, application no. 1, preferably a homolog or functional equivalent as depicted in column 8 of Table II B, application no. 1, and being depicted in the same respective line as said Sll1545, and preferably the activity is increased mitochondrial.

Accordingly, in one embodiment, the molecule which activity is to be increased in the process of the invention is the gene product with an activity as a “glutathione S-transferase”, preferably it is the molecule of section (a) or (b) of this paragraph.

In particular, it was observed that in plants, especially in Arabidopsis thaliana, increasing or generating the activity of a gene product mitochondrial with the activity of a “glutathione S-transferase”, preferably being encoded by a gene comprising the nucleic acid sequence SEQ ID NO. 11423, preferably the coding region thereof, conferred the production of or the increase in methionine compared with the wild type control.

The nucleic acid sequence of Sll1917 from Synechocystis sp., e.g. as shown in the respective line in column 5 of Table I, application no. 1, has been published in Nakamura et al., Nucleic Acids Res. 28(1), 72 (2000). And the activity of the gene product thereof is the activity of coproporphyrinogen III oxidase.

Accordingly, in one embodiment, the process of the present invention for producing methionine in a non-human organism, like a microorganism or a plant or a part thereof, comprises increasing or generating the activity of a gene product with the activity of a gene product conferring the activity of “coproporphyrinogen III oxidase”, especially from Synechocystis sp. or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said Sll1917, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said Sll1917, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said Sll1917, or a functional equivalent or a homolog thereof as depicted in column 8 of Table II, application no. 1, preferably a homolog or functional equivalent as depicted in column 8 of Table II B, application no. 1, and being depicted in the same respective line as said Sll1917, and preferably the activity is increased mitochondrial.

Accordingly, in one embodiment, the molecule which activity is to be increased in the process of the invention is the gene product with an activity as a “coproporphyrinogen III oxidase”, preferably it is the molecule of section (a) or (b) of this paragraph.

In particular, it was observed that in plants, especially in Arabidopsis thaliana, increasing or generating the activity of a gene product mitochondrial with the activity of a “coproporphyrinogen III oxidase”, preferably being encoded by a gene comprising the nucleic acid sequence SEQ ID NO. 11471, preferably the coding region thereof, conferred the production of or the increase in methionine compared with the wild type control.

The nucleic acid sequence of Slr0338 from Synechocystis sp., e.g. as shown in the respective line in column 5 of Table I, application no. 1, has been published in Nakamura et al., Nucleic Acids Res. 28(1), 72 (2000). And the activity of the gene product thereof is the activity of oxidoreductase.

Accordingly, in one embodiment, the process of the present invention for producing methionine in a non-human organism, like a microorganism or a plant or a part thereof, comprises increasing or generating the activity of a gene product with the activity of a gene product conferring the activity of “oxidoreductase”, especially from Synechocystis sp. or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said Slr0338, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said Slr0338, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said Slr0338, or a functional equivalent or a homolog thereof as depicted in column 8 of Table II, application no. 1, preferably a homolog or functional equivalent as depicted in column 8 of Table II B, application no. 1, and being depicted in the same respective line as said Slr0338, and preferably the activity is increased non-targeted.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 11990, preferably the coding region thereof, conferred the production of or the increase in methionine compared with the wild type control.

The nucleic acid sequence of Slr0338 from Synechocystis sp., e.g. as shown in the respective line in column 5 of Table I, application no. 1, has been published in Nakamura et al., Nucleic Acids Res. 28(1), 72 (2000). And the activity of the gene product thereof is the activity of oxidoreductase.

Accordingly, in one embodiment, the process of the present invention for producing methionine in a non-human organism, like a microorganism or a plant or a part thereof, comprises increasing or generating the activity of a gene product with the activity of a gene product conferring the activity of “oxidoreductase”, especially from Synechocystis sp. or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said Slr0338, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said Slr0338, and preferably the activity is increased plastidic, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said Slr0338, or a functional equivalent or a homolog thereof as depicted in column 8 of Table II, application no. 1, preferably a homolog or functional equivalent as depicted in column 8 of Table II B, application no. 1, and being depicted in the same respective line as said Slr0338, and preferably the activity is increased plastidic.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product plastidic with the activity of a “oxidoreductase”, preferably being encoded by a gene comprising the nucleic acid sequence SEQ ID NO. 11990, preferably the coding region thereof, conferred the production of or the increase in methionine compared with the wild type control.

The nucleic acid sequence of Slr1655 from Synechocystis sp., e.g. as shown in the respective line in column 5 of Table I, application no. 1, has been published in Nakamura et al., Nucleic Acids Res. 28(1), 72 (2000). And the activity of the gene product thereof is the activity of Photosystem I reaction center subunit XI.

Accordingly, in one embodiment, the process of the present invention for producing methionine in a non-human organism, like a microorganism or a plant or a part thereof, comprises increasing or generating the activity of a gene product with the activity of a gene product conferring the activity of “Photosystem I reaction center subunit XI”, especially from Synechocystis sp. or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said Slr1655, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said Slr1655, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said Slr1655, or a functional equivalent or a homolog thereof as depicted in column 8 of Table II, application no. 1, preferably a homolog or functional equivalent as depicted in column 8 of Table II B, application no. 1, and being depicted in the same respective line as said Slr1655, and preferably the activity is increased mitochondrial.

Accordingly, in one embodiment, the molecule which activity is to be increased in the process of the invention is the gene product with an activity as a “Photosystem I reaction center subunit XI”, preferably it is the molecule of section (a) or (b) of this paragraph.

In particular, it was observed that in plants, especially in Arabidopsis thaliana, increasing or generating the activity of a gene product mitochondrial with the activity of a “Photosystem I reaction center subunit XI”, preferably being encoded by a gene comprising the nucleic acid sequence SEQ ID NO. 12070, preferably the coding region thereof, conferred the production of or the increase in methionine compared with the wild type control.

The nucleic acid sequence of Slr1791 from Synechocystis sp., e.g. as shown in the respective line in column 5 of Table I, application no. 1, has been published in Synechocystis Nakamura et al., Nucleic Acids Res. 28(1), 72 (2000). And the activity of the gene product thereof is the activity of phosphoadenosine phosphosulfate reductase.

Accordingly, in one embodiment, the process of the present invention for producing methionine in a non-human organism, like a microorganism or a plant or a part thereof, comprises increasing or generating the activity of a gene product with the activity of a gene product conferring the activity of “phosphoadenosine phosphosulfate reductase”, especially from Synechocystis sp. or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said Slr1791, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said Slr1791, and preferably the activity is increased plastidic, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said Slr1791, or a functional equivalent or a homolog thereof as depicted in column 8 of Table II, application no. 1, preferably a homolog or functional equivalent as depicted in column 8 of Table II B, application no. 1, and being depicted in the same respective line as said Slr1791, and preferably the activity is increased plastidic.

Accordingly, in one embodiment, the molecule which activity is to be increased in the process of the invention is the gene product with an activity as a “phosphoadenosine phosphosulfate reductase”, preferably it is the molecule of section (a) or (b) of this paragraph.

In particular, it was observed that in plants, especially in Arabidopsis thaliana, increasing or generating the activity of a gene product plastidic with the activity of a “phosphoadenosine phosphosulfate reductase”, preferably being encoded by a gene comprising the nucleic acid sequence SEQ ID NO. 12140, preferably the coding region thereof, conferred the production of or the increase in methionine compared with the wild type control.

The nucleic acid sequence of Slr2072 from Synechocystis sp., e.g. as shown in the respective line in column 5 of Table I, application no. 1, has been published in Nakamura et al., Nucleic Acids Res. 28(1), 72 (2000). And the activity of the gene product thereof is the activity of threonine dehydratase.

Accordingly, in one embodiment, the process of the present invention for producing methionine in a non-human organism, like a microorganism or a plant or a part thereof, comprises increasing or generating the activity of a gene product with the activity of a gene product conferring the activity of “threonine dehydratase”, especially from Synechocystis sp. or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said Slr2072, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said Slr2072, and preferably the activity is increased plastidic, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said Slr2072, or a functional equivalent or a homolog thereof as depicted in column 8 of Table II, application no. 1, preferably a homolog or functional equivalent as depicted in column 8 of Table II B, application no. 1, and being depicted in the same respective line as said Slr2072, and preferably the activity is increased plastidic.

Accordingly, in one embodiment, the molecule which activity is to be increased in the process of the invention is the gene product with an activity as a “threonine dehydratase”, preferably it is the molecule of section (a) or (b) of this paragraph.

In particular, it was observed that in plants, especially in Arabidopsis thaliana, increasing or generating the activity of a gene product plastidic with the activity of a “threonine dehydratase”, preferably being encoded by a gene comprising the nucleic acid sequence SEQ ID NO. 12341, preferably the coding region thereof, conferred the production of or the increase in methionine compared with the wild type control.

The nucleic acid sequence of TTC0019 from Thermus thermophilus, e.g. as shown in the respective line in column 5 of Table I, application no. 1, has been published in Henne et al., Nat. Biotechnol. 22 (5), 547 (2004). And the activity of the gene product thereof is the activity of Sec-independent protein translocase subunit.

Accordingly, in one embodiment, the process of the present invention for producing methionine in a non-human organism, like a microorganism or a plant or a part thereof, comprises increasing or generating the activity of a gene product with the activity of a gene product conferring the activity of “Sec-independent protein translocase subunit”, especially from Thermus thermophilus or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said TTC0019, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said TTC0019, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said TTC0019, or a functional equivalent or a homolog thereof as depicted in column 8 of Table II, application no. 1, preferably a homolog or functional equivalent as depicted in column 8 of Table II B, application no. 1, and being depicted in the same respective line as said TTC0019, and preferably the activity is increased non-targeted.

Accordingly, in one embodiment, the molecule which activity is to be increased in the process of the invention is the gene product with an activity as a “Sec-independent protein translocase subunit”, preferably it is the molecule of section (a) or (b) of this paragraph.

In particular, it was observed that in plants, especially in Arabidopsis thaliana, increasing or generating the activity of a gene product non-targeted with the activity of a “Sec-independent protein translocase subunit”, preferably being encoded by a gene comprising the nucleic acid sequence SEQ ID NO. 12698, preferably the coding region thereof, conferred the production of or the increase in methionine compared with the wild type control.

The nucleic acid sequence of TTC1550 from Thermus thermophilus, e.g. as shown in the respective line in column 5 of Table I, application no. 1, has been published in Henne et al., Nat. Biotechnol. 22 (5), 547 (2004). And the activity of the gene product thereof is the activity of homocitrate synthase.

Accordingly, in one embodiment, the process of the present invention for producing methionine in a non-human organism, like a microorganism or a plant or a part thereof, comprises increasing or generating the activity of a gene product with the activity of a gene product conferring the activity of “homocitrate synthase”, especially from Thermus thermophilus or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said TTC1550, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said TTC1550, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said TTC1550, or a functional equivalent or a homolog thereof as depicted in column 8 of Table II, application no. 1, preferably a homolog or functional equivalent as depicted in column 8 of Table II B, application no. 1, and being depicted in the same respective line as said TTC1550, and preferably the activity is increased non-targeted.

Accordingly, in one embodiment, the molecule which activity is to be increased in the process of the invention is the gene product with an activity as a “homocitrate synthase”, preferably it is the molecule of section (a) or (b) of this paragraph.

In particular, it was observed that in plants, especially in Arabidopsis thaliana, increasing or generating the activity of a gene product non-targeted with the activity of a “homocitrate synthase”, preferably being encoded by a gene comprising the nucleic acid sequence SEQ ID NO. 12974, preferably the coding region thereof, conferred the production of or the increase in methionine compared with the wild type control.

The nucleic acid sequence of Yal012w from Saccharomyces cerevisiae, e.g. as shown in the respective line in column 5 of Table I, application no. 1, has been published in Goffeau et al., Science 274 (5287), 546 (1996). And the activity of the gene product thereof is the activity of cystathionine-lyase.

Accordingly, in one embodiment, the process of the present invention for producing methionine in a non-human organism, like a microorganism or a plant or a part thereof, comprises increasing or generating the activity of a gene product with the activity of a gene product conferring the activity of “cystathionine-lyase”, especially from Saccharomyces cerevisiae or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said Ya1012w, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said Yal012w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said Yal012w, or a functional equivalent or a homolog thereof as depicted in column 8 of Table II, application no. 1, preferably a homolog or functional equivalent as depicted in column 8 of Table II B, application no. 1, and being depicted in the same respective line as said Yal012w, and preferably the activity is increased non-targeted.

Accordingly, in one embodiment, the molecule which activity is to be increased in the process of the invention is the gene product with an activity as a “cystathionine-lyase”, preferably it is the molecule of section (a) or (b) of this paragraph.

In particular, it was observed that in plants, especially in Arabidopsis thaliana, increasing or generating the activity of a gene product non-targeted with the activity of a “cystathionine-lyase”, preferably being encoded by a gene comprising the nucleic acid sequence SEQ ID NO. 13376, preferably the coding region thereof, conferred the production of or the increase in methionine compared with the wild type control.

The nucleic acid sequence of Ydl155w from Saccharomyces cerevisiae, e.g. as shown in the respective line in column 5 of Table I, application no. 1, has been published in Goffeau et al., Science 274 (5287), 546 (1996). And the activity of the gene product thereof is the activity of cyclin.

Accordingly, in one embodiment, the process of the present invention for producing methionine in a non-human organism, like a microorganism or a plant or a part thereof, comprises increasing or generating the activity of a gene product with the activity of a gene product conferring the activity of “cyclin”, especially from Saccharomyces cerevisiae or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said Ydl155w, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said Ydl155w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said Ydl155w, or a functional equivalent or a homolog thereof as depicted in column 8 of Table II, application no. 1, preferably a homolog or functional equivalent as depicted in column 8 of Table II B, application no. 1, and being depicted in the same respective line as said Ydl155w, and preferably the activity is increased non-targeted.

Accordingly, in one embodiment, the molecule which activity is to be increased in the process of the invention is the gene product with an activity as a “cyclin”, preferably it is the molecule of section (a) or (b) of this paragraph.

In particular, it was observed that in plants, especially in Arabidopsis thaliana, increasing or generating the activity of a gene product non-targeted with the activity of a “cyclin”, preferably being encoded by a gene comprising the nucleic acid sequence SEQ ID NO. 14171, preferably the coding region thereof, conferred the production of or the increase in methionine compared with the wild type control.

The nucleic acid sequence of Ydl159w from Saccharomyces cerevisiae, e.g. as shown in the respective line in column 5 of Table I, application no. 1, has been published in Goffeau et al., Science 274 (5287), 546 (1996). And the activity of the gene product thereof is the activity of protein kinase.

Accordingly, in one embodiment, the process of the present invention for producing methionine in a non-human organism, like a microorganism or a plant or a part thereof, comprises increasing or generating the activity of a gene product with the activity of a gene product conferring the activity of “protein kinase”, especially from Saccharomyces cerevisiae or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said Ydl159w, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said Ydl159w, and preferably the activity is increased plastidic, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said Ydl159w, or a functional equivalent or a homolog thereof as depicted in column 8 of Table II, application no. 1, preferably a homolog or functional equivalent as depicted in column 8 of Table II B, application no. 1, and being depicted in the same respective line as said Ydl159w, and preferably the activity is increased plastidic.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product plastidic with the activity of a “protein kinase”, preferably being encoded by a gene comprising the nucleic acid sequence SEQ ID NO. 14275, preferably the coding region thereof, conferred the production of or the increase in methionine compared with the wild type control.

The nucleic acid sequence of Ydl168w from Saccharomyces cerevisiae, e.g. as shown in the respective line in column 5 of Table I, application no. 1, has been published in Goffeau et al., Science 274 (5287), 546 (1996). And the activity of the gene product thereof is the activity of protein kinase.

Accordingly, in one embodiment, the process of the present invention for producing methionine in a non-human organism, like a microorganism or a plant or a part thereof, comprises increasing or generating the activity of a gene product with the activity of a gene product conferring the activity of “protein kinase”, especially from Saccharomyces cerevisiae or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said Ydl168w, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said Ydl168w, and preferably the activity is increased plastidic, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said Ydl168w, or a functional equivalent or a homolog thereof as depicted in column 8 of Table II, application no. 1, preferably a homolog or functional equivalent as depicted in column 8 of Table II B, application no. 1, and being depicted in the same respective line as said Ydl168w, and preferably the activity is increased plastidic.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product plastidic with the activity of a “protein kinase”, preferably being encoded by a gene comprising the nucleic acid sequence SEQ ID NO. 14302, preferably the coding region thereof, conferred the production of or the increase in methionine compared with the wild type control.

The nucleic acid sequence of Ydr131c from Saccharomyces cerevisiae, e.g. as shown in the respective line in column 5 of Table I, application no. 1, has been published in Goffeau et al., Science 274 (5287), 546 (1996). And the activity of the gene product thereof is the activity of F-box protein.

Accordingly, in one embodiment, the process of the present invention for producing methionine in a non-human organism, like a microorganism or a plant or a part thereof, comprises increasing or generating the activity of a gene product with the activity of a gene product conferring the activity of “F-box protein”, especially from Saccharomyces cerevisiae or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said Ydr131c, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said Ydr131c, and preferably the activity is increased plastidic, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said Ydr131c, or a functional equivalent or a homolog thereof as depicted in column 8 of Table II, application no. 1, preferably a homolog or functional equivalent as depicted in column 8 of Table II B, application no. 1, and being depicted in the same respective line as said Ydr131c, and preferably the activity is increased plastidic.

Accordingly, in one embodiment, the molecule which activity is to be increased in the process of the invention is the gene product with an activity as a “F-box protein”, preferably it is the molecule of section (a) or (b) of this paragraph.

In particular, it was observed that in plants, especially in Arabidopsis thaliana, increasing or generating the activity of a gene product plastidic with the activity of a “F-box protein”, preferably being encoded by a gene comprising the nucleic acid sequence SEQ ID NO. 14706, preferably the coding region thereof, conferred the production of or the increase in methionine compared with the wild type control.

The nucleic acid sequence of Yhl013c from Saccharomyces cerevisiae, e.g. as shown in the respective line in column 5 of Table I, application no. 1, has been published in Goffeau et al.,

Science 274 (5287), 546 (1996). And the activity of the gene product thereof is the activity of yhl013c-protein.

Accordingly, in one embodiment, the process of the present invention for producing methionine in a non-human organism, like a microorganism or a plant or a part thereof, comprises increasing or generating the activity of a gene product with the activity of a gene product conferring the activity of “yh1013c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said Yhl013c, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said Yhl013c, and preferably the activity is increased plastidic, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said Yhl013c, or a functional equivalent or a homolog thereof as depicted in column 8 of Table II, application no. 1, preferably a homolog or functional equivalent as depicted in column 8 of Table II B, application no. 1, and being depicted in the same respective line as said Yhl013c, and preferably the activity is increased plastidic.

Accordingly, in one embodiment, the molecule which activity is to be increased in the process of the invention is the gene product with an activity as a “yhl013c-protein”, preferably it is the molecule of section (a) or (b) of this paragraph.

In particular, it was observed that in plants, especially in Arabidopsis thaliana, increasing or generating the activity of a gene product plastidic with the activity of a “yhl013c-protein”, preferably being encoded by a gene comprising the nucleic acid sequence SEQ ID NO. 14715, preferably the coding region thereof, conferred the production of or the increase in methionine compared with the wild type control.

The nucleic acid sequence of YiI053w from Saccharomyces cerevisiae, e.g. as shown in the respective line in column 5 of Table I, application no. 1, has been published in Goffeau et al., Science 274 (5287), 546 (1996). And the activity of the gene product thereof is the activity of (DL)-glycerol-3-phosphatase.

Accordingly, in one embodiment, the process of the present invention for producing methionine in a non-human organism, like a microorganism or a plant or a part thereof, comprises increasing or generating the activity of a gene product with the activity of a gene product conferring the activity of “(DL)-glycerol-3-phosphatase”, especially from Saccharomyces cerevisiae or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said YiI053w, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said Yi1053w, and preferably the activity is increased plastidic, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said Yi1053w, or a functional equivalent or a homolog thereof as depicted in column 8 of Table II, application no. 1, preferably a homolog or functional equivalent as depicted in column 8 of Table II B, application no. 1, and being depicted in the same respective line as said YiI053w, and preferably the activity is increased plastidic.

Accordingly, in one embodiment, the molecule which activity is to be increased in the process of the invention is the gene product with an activity as a “(DL)-glycerol-3-phosphatase”, preferably it is the molecule of section (a) or (b) of this paragraph.

In particular, it was observed that in plants, especially in Arabidopsis thaliana, increasing or generating the activity of a gene product plastidic with the activity of a “(DL)-glycerol-3-phosphatase”, preferably being encoded by a gene comprising the nucleic acid sequence SEQ ID NO. 14769, preferably the coding region thereof, conferred the production of or the increase in methionine compared with the wild type control.

The nucleic acid sequence of YjI137c from Saccharomyces cerevisiae, e.g. as shown in the respective line in column 5 of Table I, application no. 1, has been published in Goffeau et al., Science 274 (5287), 546 (1996). And the activity of the gene product thereof is the activity of glycogenin.

Accordingly, in one embodiment, the process of the present invention for producing methionine in a non-human organism, like a microorganism or a plant or a part thereof, comprises increasing or generating the activity of a gene product with the activity of a gene product conferring the activity of “glycogenin”, especially from Saccharomyces cerevisiae or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said YjI137c, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said Yj1137c, and preferably the activity is increased plastidic, or (b) a polypeptide comprising a polypeptide, a consensus sequence or at least a polypeptide motif as shown in the respective line in column 5 of Table II or in column 8 of Table IV, application no. 1, respectively, and being depicted in the same respective line as said YjI137c, or a functional equivalent or a homolog thereof as depicted in column 8 of Table II, application no. 1, preferably a homolog or functional equivalent as depicted in column 8 of Table II B, application no. 1, and being depicted in the same respective line as said Yj1137c, and preferably the activity is increased plastidic.

Accordingly, in one embodiment, the molecule which activity is to be increased in the process of the invention is the gene product with an activity as a “glycogenin”, preferably it is the molecule of section (a) or (b) of this paragraph.

In particular, it was observed that in plants, especially in Arabidopsis thaliana, increasing or generating the activity of a gene product plastidic with the activity of a “glycogenin”, preferably being encoded by a gene comprising the nucleic acid sequence SEQ ID NO. 14821, preferably the coding region thereof, conferred the production of or the increase in methionine compared with the wild type control.

The nucleic acid sequence of Yjr130c from Saccharomyces cerevisiae, e.g. as shown in the respective line in column 5 of Table I, application no. 1, has been published in Goffeau et al., Science 274 (5287), 546 (1996). And the activity of the gene product thereof is the activity of cystathionine gamma-synthase.

Accordingly, in one embodiment, the process of the present invention for producing methionine in a non-human organism, like a microorganism or a plant or a part thereof, comprises increasing or generating the activity of a gene product with the activity of a gene product conferring the activity of “cystathionine gamma-synthase”, especially from Saccharomyces cerevisiae or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said Yjr130c, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said Yjr130c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said Yjr130c, or a functional equivalent or a homolog thereof as depicted in column 8 of Table II, application no. 1, preferably a homolog or functional equivalent as depicted in column 8 of Table II B, application no. 1, and being depicted in the same respective line as said Yjr130c, and preferably the activity is increased non-targeted.

Accordingly, in one embodiment, the molecule which activity is to be increased in the process of the invention is the gene product with an activity as a “cystathionine gamma-synthase”, preferably it is the molecule of section (a) or (b) of this paragraph.

In particular, it was observed that in plants, especially in Arabidopsis thaliana, increasing or generating the activity of a gene product non-targeted with the activity of a “cystathionine gamma-synthase”, preferably being encoded by a gene comprising the nucleic acid sequence SEQ ID NO. 14843, preferably the coding region thereof, conferred the production of or the increase in methionine compared with the wild type control.

The nucleic acid sequence of Yjr139c from Saccharomyces cerevisiae, e.g. as shown in the respective line in column 5 of Table I, application no. 1, has been published in Goffeau et al., Science 274 (5287), 546 (1996). And the activity of the gene product thereof is the activity of homoserine dehydrogenase.

Accordingly, in one embodiment, the process of the present invention for producing methionine in a non-human organism, like a microorganism or a plant or a part thereof, comprises increasing or generating the activity of a gene product with the activity of a gene product conferring the activity of “homoserine dehydrogenase”, especially from Saccharomyces cerevisiae or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said Yjr139c, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said Yjr139c, and preferably the activity is increased plastidic, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said Yjr139c, or a functional equivalent or a homolog thereof as depicted in column 8 of Table II, application no. 1, preferably a homolog or functional equivalent as depicted in column 8 of Table II B, application no. 1, and being depicted in the same respective line as said Yjr139c, and preferably the activity is increased plastidic.

Accordingly, in one embodiment, the molecule which activity is to be increased in the process of the invention is the gene product with an activity as a “homoserine dehydrogenase”, preferably it is the molecule of section (a) or (b) of this paragraph.

In particular, it was observed that in plants, especially in Arabidopsis thaliana, increasing or generating the activity of a gene product plastidic with the activity of a “homoserine dehydrogenase”, preferably being encoded by a gene comprising the nucleic acid sequence SEQ ID NO. 14885, preferably the coding region thereof, conferred the production of or the increase in methionine compared with the wild type control.

The nucleic acid sequence of Ym1084w from Saccharomyces cerevisiae, e.g. as shown in the respective line in column 5 of Table I, application no. 1, has been published in Goffeau et al., Science 274 (5287), 546 (1996). And the activity of the gene product thereof is the activity of yml084w-protein.

Accordingly, in one embodiment, the process of the present invention for producing methionine in a non-human organism, like a microorganism or a plant or a part thereof, comprises increasing or generating the activity of a gene product with the activity of a gene product conferring the activity of “yml084w-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said Yml084w, or a functional equivalent or a homolog thereof as shown in column 8 of Table 1, application no. 1, preferably the coding region thereof, particularly a homolog or functional equivalent as shown in column 8 of Table I B, application no. 1, and being depicted in the same respective line as said Yml084w, and preferably the activity is increased plastidic, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said Yml084w, or a functional equivalent or a homolog thereof as depicted in column 8 of Table II, application no. 1, preferably a homolog or functional equivalent as depicted in column 8 of Table II B, application no. 1, and being depicted in the same respective line as said Yml084w, and preferably the activity is increased plastidic.

Accordingly, in one embodiment, the molecule which activity is to be increased in the process of the invention is the gene product with an activity as a “yml084w-protein”, preferably it is the molecule of section (a) or (b) of this paragraph.

In particular, it was observed that in plants, especially in Arabidopsis thaliana, increasing or generating the activity of a gene product plastidic with the activity of a “ym1084w-protein”, preferably being encoded by a gene comprising the nucleic acid sequence SEQ ID NO. 15175, preferably the coding region thereof, conferred the production of or the increase in methionine compared with the wild type control.

The nucleic acid sequence of Yol160w from Saccharomyces cerevisiae, e.g. as shown in the respective line in column 5 of Table I, application no. 1, has been published in Goffeau et al., Science 274 (5287), 546 (1996). And the activity of the gene product thereof is the activity of yol160w-protein.

Accordingly, in one embodiment, the process of the present invention for producing methionine in a non-human organism, like a microorganism or a plant or a part thereof, comprises increasing or generating the activity of a gene product with the activity of a gene product conferring the activity of “yol160w-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said Yol160w, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said Yol160w, and preferably the activity is increased plastidic, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said Yol160w, or a functional equivalent or a homolog thereof as depicted in column 8 of Table II, application no. 1, preferably a homolog or functional equivalent as depicted in column 8 of Table II B, application no. 1, and being depicted in the same respective line as said Yol160w, and preferably the activity is increased plastidic.

Accordingly, in one embodiment, the molecule which activity is to be increased in the process of the invention is the gene product with an activity as a “yol160w-protein”, preferably it is the molecule of section (a) or (b) of this paragraph.

In particular, it was observed that in plants, especially in Arabidopsis thaliana, increasing or generating the activity of a gene product plastidic with the activity of a “yol160w-protein”, preferably being encoded by a gene comprising the nucleic acid sequence SEQ ID NO. 15179, preferably the coding region thereof, conferred the production of or the increase in methionine compared with the wild type control.

The nucleic acid sequence of Yor392w from Saccharomyces cerevisiae, e.g. as shown in the respective line in column 5 of Table I, application no. 1, has been published in Goffeau et al., Science 274 (5287), 546 (1996). And the activity of the gene product thereof is the activity of yor392w-protein.

Accordingly, in one embodiment, the process of the present invention for producing methionine in a non-human organism, like a microorganism or a plant or a part thereof, comprises increasing or generating the activity of a gene product with the activity of a gene product conferring the activity of “yor392w-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 1, preferably the coding region thereof, or a homolog or a fragment thereof, and being depicted in the same respective line as said Yor392w, or a functional equivalent or a homolog thereof as shown in column 8 of Table I, application no. 1, preferably the coding region thereof, particularly a homolog or functional equivalent as shown in column 8 of Table I B, application no. 1, and being depicted in the same respective line as said Yor392w, and preferably the activity is increased plastidic, or
(b) a polypeptide comprising a polypeptide, a consensus sequence or at least a polypeptide motif as shown in the respective line in column 5 of Table II or in column 8 of Table IV, application no. 1, respectively, and being depicted in the same respective line as said Yor392w, or a functional equivalent or a homolog thereof as depicted in column 8 of Table II, application no. 1, preferably a homolog or functional equivalent as depicted in column 8 of Table II B, application no. 1, and being depicted in the same respective line as said Yor392w, and preferably the activity is increased plastidic.

Accordingly, in one embodiment, the molecule which activity is to be increased in the process of the invention is the gene product with an activity as a “yor392w-protein”, preferably it is the molecule of section (a) or (b) of this paragraph.

In particular, it was observed that in plants, especially in Arabidopsis thaliana, increasing or generating the activity of a gene product plastidic with the activity of a “yor392w-protein”, preferably being encoded by a gene comprising the nucleic acid sequence SEQ ID NO. 15183, preferably the coding region thereof, conferred the production of or the increase in methionine compared with the wild type control.

It was further observed that increasing or generating the activity of a FCRP gene shown in Table d, e.g. a nucleic acid molecule derived from the coding region of the nucleic acid molecule shown in Table d in A. thaliana conferred the production of or the increase in fine chemical, compared with the wild type control. Thus, in one embodiment, a nucleic acid molecule, preferably the coding region thereof, indicated in Table d or its homolog as indicated in the respective line in Table I or the expression product is used in the method of the present invention to generate or to increase the production of fine chemical in a non-human organism, like a microorganism or a plant, compared with the wild type control.

In this context, the fine chemical amount in a cell, in a non-human organism, like a plant or a microorganim or a part thereof, is increased by 1%, 3%, 5%, 10%, 20%, 30%, 40%, 50%, 60%, 70%, 80%, 90%, 100%, 120, 150% or 200% or more.

The fine chemical can be contained in the organism either in its free form and/or bound to proteins or polypeptids or mixtures thereof. Accordingly, in one embodiment, the amount of the free form in a cell, in a non-human organism, like a plant or a microorganim or part thereof, is increased byl%, 3%, 5%, 10%, 20%, 30%, 40%, 50%, 60%, 70%, 80%, 90%, 100%, 120, 150% or 200% or more. Accordingly, in another embodiment, the amount of the bound fine chemical in a cell, in a non-human organism, like a plant or a microorganim or part thereof, is increased byl%, 3%, 5%, 10%, 20%, 30%, 40%, 50%, 60%, 70%, 80%, 90%, 100%, 120, 150% or 200% or more.

A protein having an activity conferring an increase in the amount or level of the fine chemical methionine, upon targeting to the plastids or mitochondria or upon non-targeting, preferably has the structure of the respective polypeptide described herein, in particular of the polypeptides comprising the consensus sequence or at least one polypeptide motifs as shown in the respective line in Table IV, application no. 1, column 8 or of the polypeptide comprising an amino acid sequence as disclosed in the respective line in Table II, application no. 1, columns 5 or 8, or homolos or fragments thereof as described herein, or is encoded by the nucleic acid molecule characterized herein or the nucleic acid molecule according to the invention, for example by the nucleic acid molecule as shown in the respective line in Table I, application no. 1, columns 5 or 8, preferably the coding region thereof, or homologs or fragments thereof and has the herein mentioned activity.

Owing to the biological activity of the proteins which are used in the process according to the invention and which are encoded by nucleic acid molecules according to the invention, it is possible to produce compositions comprising the fine chemical, i.e. an increased amount of the fine chemical free or bound, e.g amino acid compositions. Depending on the choice of the non-human organism used for the process according to the present invention, for example a microorganism or a plant, compositions or mixtures comprising the fine chemical can be produced.

The term “expression” refers to the transcription and/or translation of a codogenic gene segment or gene. As a rule, the resulting product is an mRNA or a protein.

However, expression products can also include functional RNAs such as, for example, antisense, nucleic acids, tRNAs, snRNAs, rRNAs, RNAi, siRNA, ribozymes etc. Expression may be systemic, local or temporal, for example limited to certain cell types, tissues organs or organelles or time periods.

In one embodiment, the process of the present invention comprises one or more of the following steps
(a) stabilizing a protein conferring the generation or increased expression of a protein encoded by the nucleic acid molecule of the invention or of the polypeptide of the invention, e.g. of a polypeptide having the activity selected from the group consisting of (DL)-glycerol-3-phosphatase, 2,3-dihydroxyphenylpropionate 1,2-dioxygenase, 2-oxoglutarate dehydrogenase E1 subunit, 49747384_SOYBEAN-protein, 5′-nucleotidase, acetolactate synthase small subunit, acetyl CoA carboxylase, adenosine kinase, arginine exporter protein, At1g09680-protein, At2g45420-protein, At4g32480-protein, ATP-binding component of a transport system, auxin response factor, b0012-protein, b1003-protein, b1522protein, b2032-protein, b2345-protein, b2513-protein, b2673-protein, b3246-protein, b3346-protein, b3817-protein, b4029-protein, beta-hydroxylase, bifunctional aspartokinase/homoserine dehydrogenase, calcium-dependent protein kinase, coproporphyrinogen III oxidase, cyclin, cystathionine gamma-synthase, cystathionine-lyase, dihydroxyacid dehydratase, DNA-binding protein, F-box protein, glutaredoxin, glutathione S-transferase, glycogenin, homocitrate synthase, homoserine dehydrogenase, hydrolase, L-serine dehydratase, major facilitator superfamily transporter protein, malic enzyme, membrane transport protein, monothiol glutaredoxin, monthiol glutaredoxin, oxidoreductase, peptidylprolyl cis-trans isomerase, phosphoadenosine phosphosulfate reductase , Photosystem I reaction center subunit XI, protein kinase, protein phosphatase, pyruvate kinase, Sec-independent protein translocase subunit, serine protease, serine/threonine-protein phosphatase, threonine aldolase, threonine dehydratase, threonine efflux protein, transcription factor, transport protein, uridine/cytidine kinase, valine-pyruvate transaminase, yhl013c-protein, yml084w-protein, yol160w-protein, yor392w-protein, and zinc finger protein, or of a polypeptide as indicated in the respective line in Table II, application no. 1, columns 5 or 8,or its homologs or fragments, and conferring the production of or an increase in methionine, respectively; and/or
(b) stabilizing a mRNA conferring the generation or increased expression of a FCRP, e.g.

protein encoded by the nucleic acid molecule of the invention or its homologs or fragments, or of a mRNA encoding the polypeptide of the present invention having the hereinmentioned activity selected from the group consisting of said activities mentioned in (a) and conferring the production of or an increase in methionine, respectively; and/or
(c) increasing the specific activity of a protein conferring the increased expression of a FCRP, e.g. a protein encoded by the nucleic acid molecule of the invention or of the polypeptide of the present invention having herein-mentioned methionine generating or increasing activity, respectively, e.g. of a polypeptide having the activity of a protein as indicated in the respective line in Table II, application no. 1, columns 5 or 8, or its homologs or fragments, or decreasing the inhibitiory regulation of the polypeptide of the invention; and/or
(d) generating or increasing the expression of an endogenous or artificial transcription factor mediating the expression of a protein conferring the generation or increased expression of a FCRP, e.g. a protein encoded by the nucleic acid molecule of the invention or of the polypeptide of the invention having herein-mentioned activity selected from the group consisting of said activities mentioned in (a) and conferring a methionine increasing activity, respectively, e.g. of a polypeptide having the activity of a protein as indicated in the respective line in Table II, application no. 1, columns 5 or 8 or its homologs; and/or
(e) stimulating activity of a protein conferring the increased expression of a FCRP, e.g. a protein encoded by the nucleic acid molecule of the present invention or a polypeptide of the present invention having herein-mentioned activity selected from the group consisting of said activities mentioned in (a) and conferring a methionine increasing activity, respectively, e.g. of a polypeptide having the activity of a protein as indicated in the respective line in Table II, application no. 1, columns 5 or 8, or its homologs or fragments, by adding one or more exogenous inducing factors to the non-human organism or parts thereof; and/or
(f) expressing a transgenic gene encoding a protein conferring the increased expression of a

FCRP, e.g. a polypeptide encoded by the nucleic acid molecule of the present invention or a polypeptide of the present invention, having herein-mentioned activity selected from the group consisting of said activities mentioned in (a) and conferring a methionine increasing activity, respectively, e.g. of a polypeptide having the activity of a protein as indicated in the respective line in Table II, application no. 1, columns 5 or 8, or its homologs or fragments, and/or
(g) increasing the copy number of a gene conferring the increased expression of a nucleic acid molecule encoding a FCRP, e.g. a polypeptide encoded by the nucleic acid molecule of the invention or the polypeptide of the invention having herein-mentioned activity selected from the group consisting of said activities mentioned in (a) and conferring a methionine increasing activity, respectively, e.g. of a polypeptide having the activity of a protein as indicated in the respective line in Table II, application no. 1, columns 5 or 8, or its homologs or fragments; and/or
(h) increasing the expression of the endogenous gene encoding the FCRP, polypeptide of the invention, e.g. a polypeptide encoded by the nucleic acid molecule of the invention or the polypeptide of the invention having herein-mentioned activity selected from the group consisting of said activities mentioned in (a) and conferring a methionine; increasing activity, respectively, e.g. of a polypeptide having the activity of a protein as indicated in the respective line in Table II, application no. 1, columns 5 or 8, or its homologs or fragments, by adding positive expression or removing negative expression elements, e.g. homologous recombination can be used to either introduce positive regulatory elements like for plants the 35S enhancer into the promoter or to remove repressor elements form regulatory regions. Further gene conversion methods can be used to disrupt repressor elements or to enhance to acitivty of positive elements. Positive elements can be randomly introduced in plants by T-DNA or transposon mutagenesis and lines can be identified in which the positive elements have be integrated near to a gene of the invention, the expression of which is thereby enhanced; and/or
(i) modulating growth conditions of the non-human organism in such a manner, that the expression or activity of the gene encoding the FCRP, e.g. a protein of the invention or the protein itself is enhanced, for example microorganisms or plants can be grown for example under a higher temperature regime leading to an enhanced expression of heat shock proteins, which can lead to an enhanced methionine production, respectively; and/or
(j) selecting of non-human organisms with especially high activity of the proteins of the invention from natural or from mutagenized resources and breeding them into the target organisms, e.g. the elite crops; and/or
(k) directing a protein encoded by the nucleic acid molecule of the invention or of the polypeptide of the present invention having herein-mentioned activity selected from the group consisting of said activities mentioned in (a) and conferring a methionine increasing activity, respectively, e.g. of a polypeptide having the activity of a protein as indicated in the respective line in Table II, application no. 1, columns 5 or 8, or its homologs or fragments, if for the corresponding nucleic acid molecule in the respuective line in column 6 of Table I the term “plastidic” is indicated, to the plastids by the addition of a plastidial targeting sequence or if for the corresponding nucleic acid molecule in the respective line in column 6 of Table I the term “mitochondria!” is indicated, to the mitochondria by the addition of a mitochondrial targeting sequence; and/or
(l) generating the expression of a protein encoded by the nucleic acid molecule of the invention or of the polypeptide of the present invention having herein-mentioned activity selected from the group consisting of said activities mentioned in (a) and conferring a methionine increasing activity, respectively, e.g. of a polypeptide having the activity of a protein as indicated in the respective line in Table II, application no. 1, columns 5 or 8, or its homologs or fragments, if for the corresponding nucleic acid molecule in the respective line in column 6 of Table I the term “plastidic” or “mitochondria!” is indicated, in these organelles by the stable or transient transformation, advantageously stable transformation, of organelles, preferably plastids or mitochondria, with an inventive nucleic acid sequence preferably in form of an expression cassette containing said sequence leading to the expression of the nucleic acids or polypeptides of the invention in the respective organelle; and/or
(m) generating the expression of a protein encoded by the nucleic acid molecule of the invention or of the polypeptide of the present invention having herein-mentioned activity selected from the group consisting of said activities mentioned in (a) and conferring a methionine increasing activity, respectively, e.g. of a polypeptide having the activity of a protein as indicated in the respective line in Table II, application no. 1, columns 5 or 8, or its homologs or fragments, if for the corresponding nucleic acid molecule in the respective line in column 6 of Table I the term “plastidic” or “mitochondrial ” is indicated, in these organelles by integration of a nucleic acid of the invention into the genome of the respective organelle under control of preferable a promoter selective for the respective organelle.

Preferably, said mRNA is the coding region of the nucleic acid molecule of the present invention and/or the protein conferring the increased expression of a protein encoded by the coding region of the nucleic acid molecule of the present invention alone or linked to a transit nucleic acid sequence or transit peptide encoding nucleic acid sequence or the polypeptide having the herein mentioned activity, e.g. conferring the generation of or increase of methionine, respectively, after increasing the expression or activity of the encoded polypeptide, non-targeted or in organelles such as plastids and/or mitochondria, preferably plastids, or having the activity of a polypeptide having an activity as the protein as shown in the respective line in Table II, application no. 1, column 3, or its homologs. Preferably the increase of methionine, respectively, takes place non-targeted or in plastids and/or mitochondria, preferably non-targeted or in plastids.

In general, the amount of mRNA or polypeptide in a cell or a compartment of a non-human organism correlates with the amount of encoded protein and thus with the overall activity of the encoded protein in said volume. Said correlation is not always linear, the activity in the volume is dependent on the stability of the molecules, the degradation of the molecules or the presence of activating or inhibiting co-factors. Further, product and educt inhibitions of enzymes are well known and described in Textbooks, e.g. Stryer, Biochemistry or Zinser et al. “Enzyminhibitoren”/Enzyme inhibitors”.

The activity of the above-mentioned proteins and/or poylpeptide encoded by the nucleic acid molecule of the present invention can be increased in various ways. For example, the activity in an organism or in a part thereof, like a cell or in an organelle of the cell, is increased for example via targeting of the nucleic acid sequence or the encoded gene product to an organelle such as plastids or mitochondria, preferentially to plastids, and/or increasing the gene product number, e.g. by increasing the expression rate, like introducing a stronger promoter, or by increasing the stability of the mRNA expressed, thus increasing the translation rate, and/or increasing the stability of the gene product, thus reducing the proteins decayed. Further, the activity or turnover of enzymes can be influenced in such a way that a reduction or increase of the reaction rate or a modification (reduction or increase) of the affinity to the substrate results, is reached. A mutation in the catalytic centre of an polypeptide of the invention, e.g. as enzyme, can modulate the turn over rate of the enzyme, e.g. an exchange of an amino acid in the catalytic center can lead to an increased activity of the enzyme, or the deletion of regulator binding sites can reduce a negative regulation like a feedback inhibition (or a substrate inhibition, if the substrate level is also increased). The specific activity of an enzyme of the present invention can be increased such that the turn over rate is increased or the binding of a co-factor is improved. Improving the stability of the encoding mRNA or the protein can also increase the activity of a gene product. The stimulation of the activity is also under the scope of the term “increased activity”.

Moreover, the regulation of the above-mentioned nucleic acid sequences may be modified so that gene expression is increased. This can be achieved advantageously by means of heterologous regulatory sequences or by modifying, for example mutating, the natural regulatory sequences which are present. The advantageous methods may also be combined with each other.

In general, an activity of a gene product in a non-human organism or part thereof, in particular in a plant cell or organelle of a plant cell, a plant, or a plant or a part thereof or in a microorganism can be increased by increasing the amount of the specific encoding mRNA or the corresponding protein in said organism or part thereof. “Amount of protein or mRNA” is understood as meaning the molecule number of polypeptides or mRNA molecules in an organism, a tissue, a cell or a cell compartment. “Increase” in the amount of a protein means the quantitative increase of the molecule number of said protein in a non-human organism, a cell or a cell compartment, such as an organelle like a plastid or mitochondrion, or part thereof—for example by one of the methods described herein below—in comparison to a wild type, control or reference.

The increase in molecule number amounts preferably to at least 1%, 5%, 10%, 20%, 30%, 50%, 70%, 100%, 150%, 200% or more. However, a de novo expression is also regarded as subject of the present invention.

A modification, i.e. an increase, can be caused by endogenous or exogenous factors. For example, an increase in activity in a non-human organism or a part thereof can be caused by adding a gene product or a precursor or an activator or an agonist to the media or nutrition or can be caused by introducing said subjects into an organism, transient or stable. Such an increase can be reached by the introduction of the inventive nucleic acid sequence or the encoded protein into an organism, transient or stable. Furthermore such an increase can be reached by the introduction of the inventive nucleic acid sequence, preferably the coding region thereof, or the encoded protein in the correct cell compartement, for example into organelles, such as plastids or mitochondria, either by transformation and/or targeting.

In one embodiment the increase in the amount of the fine chemical in the non-human organism or a part thereof, e.g. in a cell, an organ, an organelle etc., is achived by increasing the endogenous level of the polypeptide of the invention. Accordingly, in an embodiment of the present invention, the present invention relates to a process wherein the gene copy number of a gene encoding the polynucleotide or nucleic acid molecule of the invention is increased. Further, the endogenous level of the polypeptide of the invention can for example be increased by modifiying the transcriptional or translational regulation of the polypeptide.

In one embodiment the amount of the fine chemical in the non-human organism or part thereof can be increase by targeted or random mutagenesis of the endogenous genes of the invention. For example homologous recombination can be used to either introduce positive regulatory elements like for plants the 35S enhancer into the promoter or to remove repressor elements form regulatory regions. In addition gene conversion like methods described by Kochevenko and Willmitzer (Plant Physiol. 132 (1), 174 (2003)) and citations therein can be used to disrupt repressor elements or to enhance to acitivty of positive regulatory elements. Furthermore positive elements can be randomly introduced in (plant) genomes by T-DNA or transposon mutagenesis and lines can be screened for, in which the positive elements has be integrated near to a gene of the invention, the expression of which is thereby enhanced. The activation of plant genes by random integrations of enhancer elements has been described by Hayashi et al. (Science 258,1350 (1992)) or Weigel et al. (Plant Physiol. 122, 1003 (2000)) and others citated therein.

Reverse genetic strategies to identify insertions (which eventually carrying the activation elements) near in genes of interest have been described for various cases e.g. Krysan et al. (Plant Cell 11, 2283 (1999)); Sessions et al. (Plant Cell 14, 2985 (2002)); Young et al. (Plant Physiol. 125, 513 (2001)); Koprek et al. (Plant J. 24, 253 (2000)); Jeon et al.(Plant J. 22, 561 (2000));

Tissier et al., (Plant Cell 11, 1841 (1999)); Speulmann et al. (Plant Cell 11, 1853 (1999)). Briefly, material from all plants of a large T-DNA or transposon mutagenized plant population is harvested and genomic DNA prepared. Then the genomic DNA is pooled following specific architectures as described for example in Krysan et al. (Plant Cell 11, 2283 (1999)). Pools of genomics DNAs are then screened by specific multiplex PCR reactions detecting the combination of the insertional mutagen (e.g. T-DNA or Transposon) and the gene of interest. Therefore PCR reactions are run on the DNA pools with specific combinations of T-DNA or transposon border primers and gene specific primers. General rules for primer design can again be taken from Krysan et al. (Plant Cell 11, 2283 (1999)). Rescreening of lower levels DNA pools lead to the identifcation of individual plants in which the gene of interest is disrupted by the insertional mutagen.

The enhancement of positive regulatory elements or the disruption or weaking of negative regulatory elements can also be achieved through common mutagenesis techniques: The production of chemically or radiation mutated populations is a common technique and known to the skilled worker. Methods for plants are described by Koorneef et al. 1982 and the citations therein and by Lightner and Caspar in “Methods in Molecular Biology” Vol 82. These techniques usually induce pointmutations that can be identified in any known gene using methods such as TILLING (Colbert et al., Plant Physiol.126 (2), 480 (2001)). One can also envisage to introduce nucleic acids sequences, encoding organelle- ,such as plastidal- or mitochondrial-targeting signals, like for example present in Table a or Table b, respectively, by homologous recombination or other methods of site specific integration, into the genome in that way, that an endogenous gene is functionally fused to the targeting sequence and the protein is redirected to the plastids or mitochondria, respectively. Eventually the integration can also occur randomly and the desired fusion event is selected for.

Accordingly, the expression level can be increased if the endogenous genes encoding a polypeptide conferring an increased expression of the polypeptide of the present invention, in particular genes comprising the nucleic acid molecule of the present invention, are modified via homologous recombination, Tilling approaches or gene conversion. It is also possible to add as mentioned herein targeting sequences to the inventive nucleic acid sequences.

Regulatory sequences, preferably in addition to a targeting sequence or part thereof (if present), can be operatively linked to the coding region of an endogenous nucleic acid and control its transcription and translation or the stability or decay of the encoding mRNA or the expressed protein. In order to modify and control the expression, promoter, UTRs, splicing sites, processing signals, polyadenylation sites, terminators, enhancers, repressors, post transcriptional or posttranslational modification sites can be changed, added or amended. For example, the activation of plant genes by random integrations of enhancer elements has been described by Hayashi et al. (Science 258, 1350 (1992)) or Weigel et al. (Plant Physiol. 122, 1003 (2000)) and others citated therein. For example, the expression level of the endogenous protein can be modulated by replacing the endogenous promoter with a stronger transgenic promoter or by replacing the endogenous 3′UTR with a 3′UTR, which provides more stablitiy without amending the coding region. Further, the transcriptional regulation can be modulated by introduction of an artifical transcription factor as described in the examples. Alternative promoters, terminators and UTR are described below.

The activation of an endogenous polypeptide having above-mentioned activity, e.g. having the activity of a protein as shown in the respective line in Table II, application no. 1, column 3 or of the polypeptide of the invention, e.g. conferring the increase of the fine chemical methionine, respectively, by increase of expression or activity in the cytoplasm, and/or in the cytosol, and/or in an organelle, such as plastids or mitochondria, can also be increased by introducing a synthetic transcription factor, which binds close to the coding region of the gene encoding the protein as shown in the respective line in Table II, application no. 1, column 5 or 8, or homologs or fragments thereof, and activates its transcription. A chimeric zinc finger protein can be constructed, which comprises a specific DNA-binding domain and an activation domain as e.g. the VP16 domain of Herpes Simplex virus. The specific binding domain can bind to the regulatory region of the gene encoding the protein as shown in the respective line in Table II, application no. 1, column 5 or 8, or homologs or fragments thereof. The expression of the chimeric transcription factor in a non-human organism, in particular in a plant, leads to a specific expression of the protein as shown in the respective line in Table II, application no. 1, column 5 or 8, or homologs or fragments thereof, see e.g. in W001/52620, Oriz, Proc. Natl. Acad. Sci. USA 99, 13290 (2002), or Guan, Proc. Natl. Acad. Sci. USA 99, 13296 (2002).

In one further embodiment of the process according to the invention, organisms are used in which one of the above-mentioned genes, or one of the above-mentioned nucleic acids, is mutated in a way that the activity of the encoded gene products is less influenced by cellular factors, or not at all, in comparison with the unmutated proteins. For example, well known regulation mechanisms of enzymic activity are substrate inhibition or feed back regulation mechanisms. Ways and techniques for the introduction of substitution, deletions and additions of one or more bases, nucleotides or amino acids of a corresponding sequence are described herein below in the corresponding paragraphs and the references listed there, e.g. in Sambrook et al., Molecular Cloning, Cold Spring Habour, N.Y., 1989. The person skilled in the art will be able to identify regulation domains and binding sites of regulators by comparing the sequence of the nucleic acid molecule of the present invention or the expression product thereof with the state of the art by computer software means which comprise algorithms for the identifying of binding sites and regulation domains or by introducing into a nucleic acid molecule or in a protein systematically mutations and assaying for those mutations which will lead to an increased specifiy activity or an increased activity per volume, in particular per cell.

It can therefore be advantageous to express in a non-human organism a nucleic acid molecule of the invention or a polypeptide of the invention derived from a evolutionary distantly related organism, as e.g. using a prokaryotic gene in a eukaryotic host, as in these cases the regulation mechanism of the host cell may not weaken the activity (cellular or specific) of the gene or its expression product.

In another embodiment it can be adavantageous to express in a non-human organism a nucleic acid molecule of the invention or a polypeptide of the invention in a compartment by the use of a respective targeting sequence, in which it is naturally not expressed, as in these cases the regulation mechanism of the host cell may not weaken the activity (cellular or specific) of the gene or its expression product.

The mutation is introduced in such a way that the production of the fine chemical is not adversely affected by e.g. undesired mutations.

Less influence on the regulation of a gene or its gene product is understood as meaning a reduced regulation of the enzymatic or biological activity leading to an increased specific or cellular activity of the gene or its product. An increase of the enzymatic activity is understood as meaning an enzymatic or biological activity, which is increased by at least 10%, 20%, 30% 40%, 50%, 60% or 70% in comparison with the starting organism. This leads to an increased productivity of the desired fine chemical(s).

Owing to the introduction of a gene or a plurality of genes conferring the expression of the nucleic acid molecule of the invention or the polypeptide of the invention, for example the nucleic acid construct mentioned below, or encoding the protein as shown in the respective line in Table II, application no. 1, column 5 or 8, or homologs or fragments thereof, into a non-human organism alone or in combination with other genes, it is possible not only to increase the biosynthetic flux towards the end product, but also to increase, modify or create de novo an advantageous, preferably novel metabolites composition in the non-human organism, e.g. an advantageous composition comprising a higher content of (from a viewpoint of nutrional physiology limited) methionine and if desired other amino acids, and/or other metabolites, in free or bound form.

In another embodiment the composition comprises further higher amounts of metabolites positively affecting or lower amounts of metabolites negatively affecting the nutrition or health of animals or humans provided with said compositions or non-human organisms of the invention or parts thereof. Likewise, the number or activity of further genes which are required for the import or export of nutrients or metabolites, including amino acids, fatty acids, vitamins etc. or its precursors, required for the cell's biosynthesis of the fine chemical may be increased so that the concentration of necessary or relevant precursors, cofactors or intermediates within the cell(s) or within the corresponding storage compartments is increased. Owing to the increased or novel generated activity of the polypeptide of the invention or owing to the increased number of nucleic acid sequences of the invention and/or to the modulation of further genes which are involved in the biosynthesis of the fine chemical, e.g. by increasing the acitivty of enzymes synthizing precursors or by destroying the activity of one or more genes which are involved in the breakdown of the fine chemical, it is possible to increase the yield, production and/or production efficiency of the fine chemical in the host organism, such as plants or microorganims.

Accordingly, in one embodiment, the process according to the invention relates to a process, which comprises:
(a) providing a non-human organism, preferably a microorganism, a plant cell, a plant tissue, a plant or a part thereof, more preferably a microorganism, a plant tissue, a plant or a part thereof;
(b) increasing the activity of a protein as shown in the respective line in Table II, application no. 1, column 3 or of a polypeptide being encoded by the respective nucleic acid molecule of the present invention and described below, e.g. conferring an increase of the fine chemical methionine, respectively, in the non-human organism, preferably in the microorganism, the plant cell, the plant tissue, the plant or a part thereof, more preferably a microorganism, a plant tissue, a plant or a part thereof, especially cytoplasmic or in an organelle, like plastids or mitochondria,
(c) growing the non-human organism, preferably the microorganism, the plant cell, the plant tissue, the plant or a part thereof, under conditions which permit the production of said fine chemical in the non-human organism, preferably the microorganism, the plant cell, the plant tissue, the plant or a part thereof; and
(d) if desired, recovering, optionally isolating, said fine chemical, in free and/or bound form, and, optionally further free and/or bound amino acids, and/or oher metabolites synthetized by the non-human organism, the microorganism, the plant cell, the plant tissue, the plant or a part thereof.

After the above-described increasing (which as defined above also encompasses the generating of an activity in a non-human organism, i.e. a de novo activity), for example after the introduction and the expression of the nucleic acid molecules of the invention or described in the methods or processes according to the invention, the organism according to the invention, advantageously, a microorganism, a non-human animal, a plant, plant or animal tissue or plant or animal cell, is grown and subsequently harvested.

Suitable non-human organisms or host organisms (transgenic organism) for the nucleic acid molecule used according to the invention and for the inventive process, the nucleic acid construct or the vector (both as described below) are, in principle, all organisms which are capable of synthesizing the fine chemical, and which are suitable for the activation, introduction or stimulation of recombinant genes. Examples which may be mentioned are transgenic plants, transgenic microorganisms such as fungi, bacteria, yeasts, alga or diatom preferably alga. Preferred organisms are those which are naturally capable of synthesizing the fine chemical in substantial amounts, like fungi, yeasts, bacteria or plants preferably alga and plants.

In the event that the transgenic non-human organism is a microorganism, such as a eukaryotic organism, for example a fungus, an alga, diatom or a yeast, in particular a fungus, alga, diatom or yeast selected from the families Tuberculariaceae, Adelotheciaceae, Dinophyceae, Ditrichaceae or Prasinophyceae. Preferred non-human organisms are microorganisms such as green algae or plants. After the growing phase, the organisms can be harvested.

The non-human organism, in particular the microorganism, plant or part thereof, is advantageously grown in such a way that it is not only possible to recover, if desired to isolate the free or bound fine chemical or the free and bound fine chemical but as option it is also possible to produce, recover and, if desired to isolate, other free or/and bound amino acids, in particular threonine and/or lysine, and/or other metabolites. For example Galili et al., Transgenic Res. 9 (2) 137 (2000) describes that the heterologous expression of a bacterial gene for the amino acid biosynthesis confers the increase of free as well as of protein-bound amino acids.

The organisms are harvested in a manner known per se and appropriate for the particular organism. Microorganisms such as bacteria, mosses, yeasts and fungi or plant cells which are cultured in liquid media by fermentation may be removed, for example, by centrifugation, decanting or filtration. Plants are grown on solid media in a manner known per se and harvested accordingly.

Preferred microorganisms are selected from the group consisting of Charophyceae such as the genera Chara, Nitella e.g. the species Chara globularis, Chara vulgaris, Nitella flexilis, Chlorophyceae such as the genera Acrosiphonia, Spongomorpha, Urospora, Bryopsis, Pseudobryopsis, Trichosolen, Dichotomosiphon, Caulerpa, Rhipilia, Blastophysa, Avrainvillea, Chlorodesmis, Codium, Espera, Halicystis, Halimeda, Penicillus, Pseudocodium, Rhipiliopsis, Rhipocephalus, Tydemania, Udotea, Derbesia, Acrochaete, Aphanochaete, Bolbocoleon, Chaetobolus, Chaetonema, Chaetophora, Chlorotylium, Desmococcus, Draparnaldia, Draparnaldiopsis, Ectochaete, Endophyton, Entocladia, Epicladia, Internoretia, Microthamnion, Ochlochaete, Phaeophila, Pilinella, Pringsheimiella, Protoderma, Pseudendoclonium, Pseudodictyon, Pseudopringsheimia, Pseudulvella, Schizomeris, Stigeoclonium, Thamniochaete, Ulvella, Pilinia, Tellamia, Helicodictyon, Actidesmium, Ankyra, Characium, Codiolum, Sykidion, Keratococcus, Prototheca, Bracteacoccus Chlorococcum, Excentrosphaera, Hormidium, Oophila, Schroederia, Tetraedron, Trebouxia Chlorosarcinopsis, Gomphonitzschia, Coccomyxa, Dactylothece, Di\acute{0}genes, Disporá, Gloeocystis, Mycanthococcus, Ourococcus, Coelastrum, Dicranochaete, Botryococcus, Dictyosphaerium, Dimorphococcus, Chlorochytrium, Kentrosphaera, Phyllobium, Gomontia, Hormotila, Euastropsis, Hydrodictyon, Pectodictyon, Pediastrum, Sorastrum, Tetrapedia, Acanthosphaera, Echinosphaerella, Echinosphaeridium, Errerella, Gloeoactinium, Golenkeniopsis Golenkinia, Micractinium, Ankistrodesmus, Chlorella, Chodatella, Closteriopsis, Cryocystis, Dactylococcus, Dematractum, Eremosphaera, Eutetramorus, Franceia, Glaucocystis, Gloeotaenium, Kirchneriella, Lagerheimiella, Monoraphidium, Nannochloris, Nephrochlamys, Nephrocytium, Oocystis, Oonephris, Pachycladon, Palmellococcus, Planktosphaeria, Polyedriopsis, Pseudoraciborskia, Quadrigula, Radiococcus, Rochiscia, Scotiella, Selanastrum, Thorakochloris, Treubaria, Trochiscia, Westella, Zoochlorella, Ostreobium, Phyllosiphon, Protosiphon, Rhodochytrium, Actinastrum, Coronastrum, Crucigenia, Dictymocystis, Enallax, Scenedesmus, Selenastrum, Tetradesmus, Tetrallantos, Tetrastrum, Chlorosarcina, Anadyomene, Valoniopsis, Ventricaria, Basicladia, Chaetomorpha, Cladophora, Lola Pithophora Rhizoclonium Chaetosphaeridium, Conochaete, Coleochaete, Oligochaetophora, Polychaetophora, Cylindrocapsa, Gongrosira, Protococcus, Acetabularia, Batophora, Bornetella, Dasycladus, Halicoryne, Neomeris, Elakatothrix, Raphidonema, Microspora, Bulbochaete, Oedocladium, Oedogonium, Prasiola, Rosenvingiella, Schizogonium, Apjohnia, Chamaedoris, Cladophoropsis, Siphonocladus, Spongocladia, Boergesenia, Boodlea, Cystodictyon, Dictyosphaeria, Ernodesmis, Microdictyon, Struvea, Valonia, Sphaeroplea, Malleochloris, Stylosphaeridium, Gloeococcus, Palmella, Palmodictyon, Palmophyllum, Pseudospherocystis, Sphaerocystis, Urococcus, Apiocystis, Chaetopeltis, Gemellicystis, Paulschulzia, Phacomyxa, Pseudotetraspora, Schizochlamys, Tetraspora, Cephaleuros, Ctenocladus, Epibolium, Leptosira, Trentepohlia, Diplochaete, Monostroma, Binuclearia, Geminella, Klebsormidium, Planetonema, Radiofilum, Stichococcus, Ulothrix, Uronema, Blidingia, Capsosiphon, Chloropelta, Enteromorpha, Percursaria, Ulva, Ulvaria, Brachiomonas, Carteria, Chlainomonas, Chlamydomonas, Chlamydonephris, Chlorangium, Chlorogonium, Cyanidium, Fortiella, Glenomonas, Gloeomonas, Hyalogonium, Lobomonas Polytoma, Pyramichlamys, Scourfieldia, Smithsonimonas, Sphaerellopsis, Sphenochloris, Spirogonium, Collodictyon, Dunaliella, Haematococcus, Stephanosphaera, Coccomonas, Dysmorphococcus, Phacotus, Pteromonas, Thoracomonas, Wislouchiella, Mascherina, Pyrobotrys, Spondylomorum, Eudorina, Gonium, Oltmannsiella, Pandorina, Platydorina, Pleodorina, Stephanoon, Volvox, Volvulina, Actinotaenium, Arthrodesmus, Bambusina Closterium, Cosmarium, Desmidium, Euastrum, Groenbladia, Hyalotheca, Micrasterias, Penium, Phymatodocis, Pleurotaenium, Sphaerozosma, Spinoclosterium, Spinocosmarium, Spondylosium, Staurastrum, Tetmemorus, Triploceras, Xanthidium, Cylindrocystis, Genicularia, Gonatozygon, Mesotaenium, Netrium, Roya, Spirotaenia, Cosmocladium, Debarya, Docidium, Euastridium, Hallasia, Mougeotia, Mougeotiopsis, Sirogonium, Spirogyra, Staurodesmus, Teilingia, Zygnema, Zygogonium, e.g. the species Caulerpa taxifolia, Prototheca wickerhamii, Ankistrodesmus falcatus, Chlorella ellipsoidea, Chlorella pyrenoidosa, CloreIla sorokiniana, Chlorella vulgaris, Scenedesmus obliquus, Scenedesmus quadricauda, Selenastrum capricornutum, Selenastrum undecimnotata, Cladophora glomerata, Chlamydomonas eugametos, Chlamydomonas reinhardtii, Cyanidium caldarium, Dunaliella salina, Dunaliella tertiolecta, Euglena gracilis, Haematococcus pluvialis, Coniugatophyceae, Prasinophyceae Trebouxiophyceae, Ulvophyceae, Chlorodendraceae, Pedinomonadales, Halosphaeraceae, Pterospermataceae, Monomastigaceae, Pyramimonadaceae, Chlorodendraceae such as the genera Prasinocladus e.g. the species Prasinocladus ascus, Halosphaeraceae, Pedinomonadales, Pedinomonadaceae such as the genera Pedinomonas, Pterospermataceae such as the genera Pachysphaera, Pterosperma, Halosphaera, Pyramimonas, Bacillariophyceae, Chrysophyceae, Craspedophyceae, Euglenophyceae, Prymnesiophyceae, Phaeophyceae, Dinophyceae, Rhodophyceae, Xanthophyceae, Prasinophyceae such as the genera Nephroselmis, Prasinococcus, Scherffelia, Tetraselmis, Mantoniella, Ostreococcus e.g. the species Nephroselmis olivacea, Prasinococcus capsulatus, Scherffelia dubia, Tetraselmis chui, Tetraselmis suecica, Mantoniella squamata or Ostreococcus tauri.

Preferred plants are monocotyledonous or dicotyledonous plants, preferably selected from the group consisting of Anacardiaceae such as the genera Pistacia, Mangifera, Anacardium e.g. the species Pistacia vera [pistachios, Pistazie], Mangifer indica [Mango] or Anacardium occidentale [Cashew]; Asteraceae such as the genera Calendula, Carthamus, Centaurea, Cichorium, Cynara, Helianthus, Lactuca, Locusta, Tagetes, Valeriana e.g. the species Calendula officinalis [Marigold], Carthamus tinctorius [safflower], Centaurea cyanus [cornflower], Cichorium intybus [blue daisy], Cynara scolymus [Artichoke], Helianthus annus [sunflower], Lactuca sativa, Lactuca crispa, Lactuca esculenta, Lactuca scariola L. ssp. sativa, Lactuca scariola L. var. integrate, Lactuca scariola L. var. integrifolia, Lactuca sativa subsp. romana, Locusta communis, Valeriana locusta [lettuce], Tagetes lucida, Tagetes erecta or Tagetes tenuifolia [Marigold]; Apiaceae such as the genera Daucus e.g. the species Daucus carota [carrot]; Betulaceae such as the genera Corylus e.g. the species Corylus avellana or Corylus colurna [hazelnut]; Boraginaceae such as the genera Borago e.g. the species Borago officinalis [borage]; Brassicaceae such as the genera Brassica, Melanosinapis, Sinapis, Arabadopsis e.g. the species Brassica napus, Brassica rapa ssp. [canola, oilseed rape, turnip rape], Sinapis arvensis Brassica juncea, Brassica juncea var. juncea, Brassica juncea var. crispifolia, Brassica juncea var. foliosa, Brassica nigra, Brassica sinapioides, Melanosinapis communis [mustard], Brassica oleracea [fodder beet] or Arabidopsis thaliana; Bromeliaceae such as the genera Anana, Bromelia e.g. the species Anana comosus, Ananas ananas or Bromelia comosa [pineapple]; Caricaceae such as the genera Carica e.g. the species Carica papaya [papaya]; Cannabaceae such as the genera Cannabis e.g. the species Cannabis sative [hemp], Convolvulaceae such as the genera Ipomea, Convolvulus e.g. the species Ipomoea batatus, Ipomoea pandurata, Convolvulus batatas, Convolvulus tiliaceus, Ipomoea fastigiata, Ipomoea tiliacea, Ipomoea triloba or Convolvulus panduratus [sweet potato, Man of the Earth, wild potato], Chenopodiaceae such as the genera Beta, i.e. the species Beta vulgaris, Beta vulgaris var. altissima, Beta vulgaris var. Vulgaris, Beta maritima, Beta vulgaris var. perennis, Beta vulgaris var. conditiva or Beta vulgaris var. esculenta [sugar beet]; Cucurbitaceae such as the genera Cucubita e.g. the species Cucurbita maxima, Cucurbita mixta, Cucurbita pepo or Cucurbita moschata [pumpkin, squash]; Elaeagnaceae such as the genera Elaeagnus e.g. the species Olea europaea [olive]; Ericaceae such as the genera Kalmia e.g. the species Kalmia latifolia, Kalmia angustifolia, Kalmia microphylla, Kalmia polifolia, Kalmia occidentalis, Cistus chamaerhodendros or Kalmia lucida [American laurel, broad-leafed laurel, calico bush, spoon wood, sheep laurel, alpine laurel, bog laurel, western bog-laurel, swamp-laurel]; Euphorbiaceae such as the genera Manihot, Janipha, Jatropha, Ricinus e.g. the species Manihot utilissima, Janipha manihot, Jatropha manihot, Manihot aipil, Manihot dulcis, Manihot manihot, Manihot melanobasis, Manihot esculenta [manihot, arrowroot, tapioca, cassava] or Ricinus communis [castor bean, Castor Oil Bush, Castor Oil Plant, Palma Christi, Wonder Tree]; Fabaceae such as the genera Pisum, Albizia, Cathormion, Feuillea, Inga, Pithecolobium, Acacia, Mimosa, Medicajo, Glycine, Dolichos, Phaseolus, Soja e.g. the species Pisum sativum, Pisum arvense, Pisum humile [pea], Albizia berteriana, Albizia julibrissin, Albizia lebbeck, Acacia berteriana, Acacia littoralis, Albizia berteriana, Albizzia berteriana, Cathormion berteriana, Feuillea berteriana, Inga fragrans, Pithecellobium berterianum, Pithecellobium fragrans, Pithecolobium berterianum, Pseudalbizzia berteriana, Acacia julibrissin, Acacia nemu, Albizia nemu, Feuilleea julibrissin, Mimosa julibrissin, Mimosa speciosa, Sericanrda julibrissin, Acacia lebbeck, Acacia macrophylla, Albizia lebbek, Feuilleea lebbeck, Mimosa lebbeck, Mimosa speciosa [bastard logwood, silk tree, East Indian Walnut], Medicago sativa, Medicago falcata, Medicago varia [alfalfa], Glycine max [soybean], Dolichos soja, Glycine gracilis, Glycine hispida, Phaseolus max or Soja hispida ; Geraniaceae such as the genera Pelargonium, Cocos, Oleum e.g. the species Cocos nucifera, Pelargonium grossularioides or Oleum cocois [coconut]; Gramineae such as the genera Saccharum e.g. the species Saccharum officinarum; Juglandaceae such as the genera Juglans, Wallia e.g. the species Juglans regia, Juglans ailanthifolia, Juglans sieboldiana, Juglans cinerea, Wallia cinerea, Juglans bixbyi, Juglans californica, Juglans hindsii, Juglans intermedia, Juglans jamaicensis, Juglans major, Juglans microcarpa, Juglans nigra or Wallia nigra [walnut, black walnut, common walnut, persian walnut, white walnut, butternut, black walnut]; Lauraceae such as the genera Persea, Laurus e.g. the species laurel Laurus nobilis [bay, laurel, bay laurel, sweet bay], Persea americana Persea americana, Persea gratissima or Persea persea [avocado]; Leguminosae such as the genera Arachis e.g. the species Arachis hypogaea [peanut]; Linaceae such as the genera Linum, Adenolinum e.g. the species Linum usitatissimum, Linum humile, Linum austriacum, Linum bienne, Linum angustifolium, Linum catharticum, Linum flavum, Linum grandiflorum, Adenolinum grandiflorum, Linum lewisii, Linum narbonense, Linum perenne, Linum perenne var. lewisii, Linum pratense or Linum trigynum [flax, linseed]; Lythrarieae such as the genera Punica e.g. the species Punica granatum [pomegranate]; Malvaceae such as the genera Gossypium e.g. the species Gossypium hirsutum, Gossypium arboreum, Gossypium barbadense, Gossypium herbaceum or Gossypium thurberi [cotton]; Musaceae such as the genera Musa e.g. the species Musa nana, Musa acuminata, Musa paradisiaca, Musa spp. [banana]; Onagraceae such as the genera Camissonia, Oenothera e.g. the species Oenothera biennis or Camissonia brevipes [primrose, evening primrose]; Palmae such as the genera Elacis e.g. the species Elaeis guineensis [oil plam]; Papaveraceae such as the genera Papaver e.g. the species Papaver orientale, Papaver rhoeas, Papaver dubium [poppy, oriental poppy, corn poppy, field poppy, shirley poppies, field poppy, long-headed poppy, long-pod poppy]; Pedaliaceae such as the genera Sesamum e.g. the species Sesamum indicum [sesame]; Piperaceae such as the genera Piper, Artanthe, Peperomia, Steffensia e.g. the species Piper aduncum, Piper amalago, Piper angustifolium, Piper auritum, Piper betel, Piper cubeba, Piper longum, Piper nigrum, Piper retrofractum, Artanthe adunca, Artanthe elongata, Peperomia elongata, Piper elongatum, Steffensia elongata. [Cayenne pepper, wild pepper]; Poaceae such as the genera Hordeum, Secale, Avena, Sorghum, Andropogon, Holcus, Panicum, Oryza, Zea, Triticum e.g. the species Hordeum vulgare, Hordeum jubatum, Hordeum murinum, Hordeum secalinum, Hordeum distichon Hordeum aegiceras, Hordeum hexastichon., Hordeum hexastichum, Hordeum irregulare, Hordeum sativum, Hordeum secalinum [barley, pearl barley, foxtail barley, wall barley, meadow barley], Secale cereale [rye], Avena sativa, Avena fatua, Avena byzantina, Avena fatua var. sativa, Avena hybrida [oat], Sorghum bicolor, Sorghum halepense, Sorghum saccharatum, Sorghum vulgare, Andropogon drummondii, Holcus bicolor, Holcus sorghum, Sorghum aethiopicum, Sorghum arundinaceum, Sorghum caffrorum, Sorghum cernuum, Sorghum dochna, Sorghum drummondii, Sorghum durra, Sorghum guineense, Sorghum lanceolatum, Sorghum nervosum, Sorghum saccharatum, Sorghum subglabrescens, Sorghum verticilliflorum, Sorghum vulgare, Holcus halepensis, Sorghum miliaceum millet, Panicum militaceum [Sorghum, millet], Oryza sativa, Oryza latifolia [rice], Zea mays [corn, maize] Triticum aestivum, Triticum durum, Triticum turgidum, Triticum hybernum, Triticum macha, Triticum sativum or Triticum vulgare [wheat, bread wheat, common wheat], Proteaceae such as the genera Macadamia e.g. the species Macadamia intergrifolia [macadamia]; Rubiaceae such as the genera Coffea e.g. the species Cofea spp., Coffea arabica, Coffea canephora or Coffea liberica [coffee]; Scrophulariaceae such as the genera Verbascum e.g. the species Verbascum blattaria, Verbascum chaixii, Verbascum densiflorum, Verbascum lagurus, Verbascum longifolium, Verbascum lychnitis, Verbascum nigrum, Verbascum olympicum, Verbascum phlomoides, Verbascum phoenicum, Verbascum pulverulentum or Verbascum thapsus [mullein, white moth mullein, nettle-leaved mullein, dense-flowered mullein, silver mullein, long-leaved mullein, white mullein, dark mullein, greek mullein, orange mullein, purple mullein, hoary mullein, great mullein]; Solanaceae such as the genera Capsicum, Nicotiana, Solanum, Lycopersicon e.g. the species Capsicum annuum, Capsicum annuum var. glabriusculum, Capsicum frutescens [pepper], Capsicum annuum [paprika], Nicotiana tabacum, Nicotiana alata, Nicotiana attenuata, Nicotiana glauca, Nicotiana langsdorffii, Nicotiana obtusifolia, Nicotiana quadrivalvis, Nicotiana repanda, Nicotiana rustica, Nicotiana sylvestris [tobacco], Solanum tuberosum [potato],Solanum melongena [egg-plant] (Lycopersicon esculentum, Lycopersicon lycopersicum, Lycopersicon pyriforme, Solanum integrifolium or Solanum lycopersicum [tomato]; Sterculiaceae such as the genera Theobroma e.g. the species Theobroma cacao [cacao]; Theaceae such as the genera Camellia e.g. the species Camellia sinensis) [tea].

Particular preferred plants are monocotyledonous or dicotyledonous plants selected from the group consisting of Asteraceae such as the genera Helianthus, Tagetes e.g. the species Helianthus annus [sunflower], Tagetes lucida, Tagetes erecta or Tagetes tenuifolia [Marigold]; Brassicaceae such as the genera Brassica, Arabadopsis e.g. the species Brassica napus, Brassica rapa ssp. [canola, oilseed rape, turnip rape] or Arabidopsis thaliana; Fabaceae such as the genera Glycine e.g. the species Glycine max [soybean] or Soja hispida; Linaceae such as the genera Linum e.g. the species Linum usitatissimum, [flax, linseed]; Poaceae such as the genera Hordeum, Secale, Avena, Sorghum, Oryza, Zea, Triticum e.g. the species Hordeum vulgare [barley], Secale cereale [rye], Avena sativa, Avena fatua, Avena byzantina, Avena fatua var. sativa, Avena hybrida [oat], Sorghum bicolor [Sorghum, millet], Oryza sativa, Oryza latifolia [rice], Zea mays [corn, maize], Triticum aestivum, Triticum durum, Triticum turgidum, Triticum hybernum, Triticum macha, Triticum sativum or Triticum vulgare [wheat, bread wheat, common wheat]; Solanaceae such as the genera Solanum, Lycopersicon e.g. the species Solanum tuberosum [potato], Lycopersicon esculentum, Lycopersicon lycopersicum, Lycopersicon pyriforme, Solanum integrifolium or Solanum lycopersicum [tomato]; Gramineae such as the genera Saccharum e.g. the species Saccharum officinarum or Helianthus annus [sunflower] or Chenopodiaceae such as the genera Beta, i.e. Beta vulgaris var. esculenta [sugar beet].

All above-mentioned organisms can be used as host organisms and/or can be used as donor organism.

The invention is not limited to specific nucleic acids, specific polypeptides, specific cell types, specific host cells, specific conditions or specific methods etc. as such, but may vary and numerous modifications and variations therein will be apparent to those skilled in the art. It is also to be understood that the terminology used herein is for the purpose of describing specific embodiments only and is not intended to be limiting.

In another embodiment, the invention relates to a (isolated) nucleic acid molecule, which comprises a nucleic acid molecule selected from the group consisting of:
(a) a nucleic acid molecule encoding a polypeptide shown in Table II, application no. 1, column 5 or 8, preferably shown in Table II B, application no. 1, column 8, or a homolog or a fragment thereof;
(b) a nucleic acid molecule shown in Table I, application no. 1, column 5 or 8, preferably shown in Table I B, application no. 1, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 1, preferably in column 8 of Table II B, application no. 1;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 1, preferably shown in column 8 of Table I B, application no. 1, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 1;
(i) a nucleic acid molecule encoding a polypeptide having the activity represented by a respective protein as depicted in column 5 of Table II, application no. 1;
(j) a nucleic acid molecule comprising a polynucleotide which is obtained by amplifying a cDNA library or a genomic library using the primers in Table III, application no. 1, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 1.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II application no. 1 and confers a generation or an increase of the respective fine chemical in a non-human organism or a part thereof as compared to a corresponding wild-type non-human organism or part thereof.

In another embodiment, the invention relates to a (isolated) nucleic acid molecule, which comprises a nucleic acid molecule selected from the group consisting of:
(a) a nucleic acid molecule encoding a polypeptide shown in Table II, column 5 or 8, application no. 1, preferably shown in Table II A, application no. 1, in column 5 or in Table II A, application no. 1, column 8 or in Table II B, application no. 1, column 8, or a homolog or a fragment thereof;
(b) a nucleic acid molecule shown in Table I, application no. 1, column 5 or 8, preferably shown in Table I A, application no. 1, in column 5 or in Table I A, application no. 1, column 8 or in Table I B, application no. 1, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 1, preferably shown in Table II A, application no. 1, in column 5 or in Table II A, application no. 1, column 8 or in Table II B, application no. 1, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 1, preferably shown in Table I A, application no. 1, in column 5 or in Table I A, application no. 1, column 8 or in Table I B column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);

(h) a nucleic acid molecule encoding a polypeptide comprising the consensus sequence or one or more polypeptide motifs as shown in column 8 of Table IV, application no. 1;
(i) a nucleic acid molecule encoding a polypeptide having the activity represented by a respective protein as depicted in column 5 of Table II, application no. 1;
(j) a nucleic acid molecule comprising a polynucleotide which is obtained by amplifying a cDNA library or a genomic library using the primers in Table III, application no. 1, column 8; and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt,500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
   or a nucleic acid molecule comprising a sequence which is complementary thereto;
   whereby, preferably, the nucleic acid molecule according to (a), (b), (c), (d), (e), (f), (g), (h), (i), (j) or (k) distinguishes over the sequence depicted in Table I A and/or I B, application no. 1, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 1, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 1, column 5 or 8, or the coding region thereof, respectively. In a further embodiment the nucleic acid molecule does not encode the polypeptide sequence shown in Table II A and/or II B, application no. 1, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 1, column 5 or 8 and does not encode a protein of the sequence shown in Table II A and/or II B, application no. 1, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 1, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 1, column 5 or 8 but less than 100%, preferably less than 99.999%, 99.99%, 99.9%, 99%, 98%, 97%, 96% or 95% identical to the sequence shown in Table II A and/or II B, application no. 1, column 5 or 8.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 1.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II, application no. 1, and confers a generation or an increase of the respective fine chemical in a non-human organism or a part thereof as compared to a corresponding wild-type non-human organism or part thereof.

In one embodiment the invention relates to the aforementioned nucleic acid molecules, which can be isolated advantageously from yeast, fungi, viruses, algae, bacteria, for example from Charophyceae such as the genera Chara, Nitella e.g. the species Chara globularis, Chara vulgaris, Nitella flexilis, Chlorophyceae such as the genera Acrosiphonia, Spongomorpha, Urospora, Bryopsis, Pseudobryopsis, Trichosolen, Dichotomosiphon, Caulerpa, Rhipilia, Blastophysa, Avrainvillea, Chlorodesmis, Codium, Espera, Halicystis, Halimeda, Penicillus, Pseudocodium, Rhipiliopsis, Rhipocephalus, Tydemania, Udotea, Derbesia, Acrochaete, Aphanochaete, Bolbocoleon, Chaetobolus, Chaetonema, Chaetophora, Chlorotylium, Desmococcus, Draparnaldia, Draparnaldiopsis, Ectochaete, Endophyton, Entocladia, Epicladia, Internoretia, Microthamnion, Ochlochaete, Phaeophila, Pilinella, Pringsheimiella, Protoderma, Pseudendoclonium, Pseudodictyon, Pseudopringsheimia, Pseudulvella, Schizomeris, Stigeoclonium, Thamniochaete, Ulvella, Pilinia, Tellamia, Helicodictyon, Actidesmium, Ankyra, Characium, Codiolum, Sykidion, Keratococcus, Prototheca, Bracteacoccus Chlorococcum, Excentrosphaera, Hormidium, Oophila, Schroederia, Tetraedron, Trebouxia Chlorosarcinopsis, Gomphonitzschia, Coccomyxa, Dactylothece, Diógenes, Disporá, Gloeocystis, Mycanthococcus, Ourococcus, Coelastrum, Dicranochaete, Botryococcus, Dictyosphaerium, Dimorphococcus, Chlorochytrium, Kentrosphaera, Phyllobium, Gomontia, Hormotila, Euastropsis, Hydrodictyon, Pectodictyon, Pediastrum, Sorastrum, Tetrapedia, Acanthosphaera, Echinosphaerella, Echinosphaeridium, Errerella, Gloeoactinium, Golenkeniopsis Golenkinia, Micractinium, Ankistrodesmus, Chlorella, Chodatella, Closteriopsis, Cryocystis, Dactylococcus, Dematractum, Eremosphaera, Eutetramorus, Franceia, Glaucocystis, Gloeotaenium, Kirchneriella, Lagerheimiella, Monoraphidium, Nannochloris, Nephrochlamys, Nephrocytium, Oocystis, Oonephris, Pachycladon, Palmellococcus, Planktosphaeria, Polyedriopsis, Pseudoraciborskia, Quadrigula, Radiococcus, Rochiscia, Scotiella, Selanastrum, Thorakochloris, Treubaria, Trochiscia, Westella, Zoochlorella, Ostreobium, Phyllosiphon, Protosiphon, Rhodochytrium, Actinastrum, Coronastrum, Crucigenia, Dictymocystis, Enallax, Scenedesmus, Selenastrum, Tetradesmus, Tetrallantos, Tetrastrum, Chlorosarcina, Anadyomene, Valoniopsis, Ventricaria, Basicladia, Chaetomorpha, Cladophora, LolaPithophoraRhizoclonium Chaetosphaeridium, Conochaete, Coleochaete, Oligochaetophora, Polychaetophora, Cylindrocapsa, Gongrosira, Protococcus, Acetabularia, Batophora, Bornetella, Dasycladus, Halicoryne, Neomeris, Elakatothrix, Raphidonema, Microspora, Bulbochaete, Oedocladium, Oedogonium, Prasiola, Rosenvingiella, Schizogonium, Apjohnia, Chamaedoris, Cladophoropsis, Siphonocladus, Spongocladia, Boergesenia, Boodlea, Cystodictyon, Dictyosphaeria, Ernodesmis, Microdictyon, Struvea, Valonia, Sphaeroplea, Malleochloris, Stylosphaeridium, Gloeococcus, Palmella, Palmodictyon, Palmophyllum, Pseudospherocystis, Sphaerocystis, Urococcus, Apiocystis, Chaetopeltis, Gemellicystis, Paulschulzia, Phacomyxa, Pseudotetraspora, Schizochlamys, Tetraspora, Cephaleuros, Ctenocladus, Epibolium, Leptosira, Trentepohlia, Diplochaete, Monostroma, Binuclearia, Geminella, Klebsormidium, Planetonema, Radiofilum, Stichococcus, Ulothrix, Uronema, Blidingia, Capsosiphon, Chloropelta, Enteromorpha, Percursaria, Ulva, Ulvaria, Brachiomonas, Carteria, Chlainomonas, Chlamydomonas, Chlamydonephris, Chlorangium, Chlorogonium, Cyanidium, Fortiella, Glenomonas, Gloeomonas, Hyalogonium, Lobomonas Polytoma, Pyramichlamys, Scourfieldia, Smithsonimonas, Sphaerellopsis, Sphenochloris, Spirogonium, Collodictyon, Dunaliella, Haematococcus, Stephanosphaera, Coccomonas, Dysmorphococcus, Phacotus, Pteromonas, Thoracomonas, Wislouchiella, Mascherina, Pyrobotrys, Spondylomorum, Eudorina, Gonium, Oltmannsiella, Pandorina, Platydorina, Pleodorina, Stephanoon, Volvox, Volvulina, Actinotaenium, Arthrodesmus, Bambusina Closterium, Cosmarium, Desmidium, Euastrum, Groenbladia, Hyalotheca, Micrasterias, Penium, Phymatodocis, Pleurotaenium, Sphaerozosma, Spinoclosterium, Spinocosmarium, Spondylosium, Staurastrum, Tetmemorus, Triploceras, Xanthidium, Cylindrocystis, Genicularia, Gonatozygon, Mesotaenium, Netrium, Roya, Spirotaenia, Cosmocladium, Debarya, Docidium, Euastridium, Hallasia, Mougeotia, Mougeotiopsis, Sirogonium, Spirogyra, Staurodesmus, Teilingia, Zygnema, Zygogonium, e.g. the species Caulerpa taxifolia, Prototheca wickerhamii, Ankistrodesmus falcatus, Chlorella ellipsoidea, Chlorella pyrenoidosa, Clorella sorokiniana, Chlorella vulgaris, Scenedesmus obliquus, Scenedesmus quadricauda, Selenastrum capricornutum, Selenastrum undecimnotata, Cladophora glomerata, Chlamydomonas eugametos, Chlamydomonas reinhardtii, Cyanidium caldarium, Dunaliella salina, Dunaliella tertiolecta, Euglena gracilis, Haematococcus pluvialis, Coniugatophyceae, Prasinophyceae Trebouxiophyceae, Ulvophyceae, Chlorodendraceae, Pedinomonadales, Halosphaeraceae, Pterospermataceae, Monomastigaceae, Pyramimonadaceae, Chlorodendraceae such as the genera Prasinocladus e.g. the species Prasinocladus ascus, Halosphaeraceae, Pedinomonadales, Pedinomonadaceae such as the genera Pedinomonas, Pterospermataceae such as the genera Pachysphaera, Pterosperma, Halosphaera, Pyramimonas, Bacillariophyceae, Chrysophyceae, Craspedophyceae, Euglenophyceae, Prymnesiophyceae, Phaeophyceae, Dinophyceae, Rhodophyceae, Xanthophyceae, Prasinophyceae such as the genera Nephroselmis, Prasinococcus, Scherffelia, Tetraselmis, Mantoniella, Ostreococcus e.g. the species Nephroselmis olivacea, Prasinococcus capsulatus, Scherffelia dubia, Tetraselmis chui, Tetraselmis suecica, Mantoniella squamata, Ostreococcus tauri, especially such as Acetobacter (subgen. Acetobacter) aceti; Acidithiobacillus ferrooxidans; Acinetobacter sp.; Actinobacillus sp; Aeromonas salmonicida; Agrobacterium tumefaciens; Aquifex aeolicus; Arcanobacterium pyogenes; Aster yellows phytoplasma; Bacillus sp.; Bifidobacterium sp.; Borrelia burgdorferi; Brevibacterium linens; Brucella melitensis; Buchnera sp.; Butyrivibrio fibrisolvens; Campylobacter jejuni; Caulobacter crescentus; Chlamydia sp.; Chlamydophila sp.; Chlorobium limicola; Citrobacter rodentium; Clostridium sp.; Comamonas testosteroni; Corynebacterium sp.; Coxiella burnetii; Deinococcus radiodurans; Dichelobacter nodosus; Edwardsiella ictaluri; Enterobacter sp.; Erysipelothrix rhusiopathiae; E. coli; Flavobacterium sp.; Francisella tularensis; Frankia sp. Cpl1; Fusobacterium nucleatum; Geobacillus stearothermophilus; Gluconobacter oxydans; Haemophilus sp.; Helicobacter pylori; Klebsiella pneumoniae; Lactobacillus sp.; Lactococcus lactis; Listeria sp.; Mannheimia haemolytica; Mesorhizobium loti; Methylophaga thalassica; Microcystis aeruginosa; Microscilla sp. PRE1; Moraxella sp. TA144; Mycobacterium sp.; Mycoplasma sp.; Neisseria sp.; Nitrosomonas sp.; Nostoc sp. PCC 7120; Novosphingobium aromaticivorans; Oenococcus oeni; Pantoea citrea; Pasteurella multocida; Pediococcus pentosaceus; Phormidium foveolarum; Phytoplasma sp.; Plectonema boryanum; Prevotella ruminicola; Propionibacterium sp.; Proteus vulgaris; Pseudomonas sp.; Ralstonia sp.; Rhizobium sp.; Rhodococcus equi; Rhodothermus marinus; Rickettsia sp.; Riemerella anatipestifer; Ruminococcus flavefaciens; Salmonella sp.; Selenomonas ruminantium; Serratia entomophila; Shigella sp.; Sinorhizobium meliloti; Staphylococcus sp.; Streptococcus sp.; Streptomyces sp.; Synechococcus sp.; Synechocystis sp. PCC 6803; Thermotoga maritima; Treponema sp.; Ureaplasma urealyticum; Vibrio cholerae; Vibrio parahaemolyticus; Xylella fastidiosa; Yersinia sp.; Zymomonas mobilis, preferably Salmonella sp. or E. coli, preferably from yeasts such as from the genera Saccharomyces, Pichia, Candida, Hansenula, Torulopsis or Schizosaccharomyces, or from plants, such as monocotyledonous or dicotyledonous plants, preferably selected from the group consisting of Anacardiaceae such as the genera Pistacia, Mangifera, Anacardium e.g. the species Pistacia vera [pistachios, Pistazie], Mangifer indica [Mango] or Anacardium occidentale [Cashew]; Asteraceae such as the genera Calendula, Carthamus, Centaurea, Cichorium, Cynara, Helianthus, Lactuca, Locusta, Tagetes, Valeriana e.g. the species Calendula officinalis [Marigold], Carthamus tinctorius [safflower], Centaurea cyanus [cornflower], Cichorium intybus [blue daisy], Cynara scolymus [Artichoke], Helianthus annus [sunflower], Lactuca sativa, Lactuca crispa, Lactuca esculenta, Lactuca scariola L. ssp. sativa, Lactuca scariola L. var. integrata, Lactuca scariola L. var. integrifolia, Lactuca sativa subsp. romana, Locusta communis, Valeriana locusta [lettuce], Tagetes lucida, Tagetes erecta or Tagetes tenuifolia [Marigold]; Apiaceae such as the genera Daucus e.g. the species Daucus carota [carrot]; Betulaceae such as the genera Corylus e.g. the species Corylus avellana or Corylus colurna [hazelnut]; Boraginaceae such as the genera Borago e.g. the species Borago officinalis [borage]; Brassicaceae such as the genera Brassica, Melanosinapis, Sinapis, Arabadopsis e.g. the species Brassica napus, Brassica rapa ssp. [canola, oilseed rape, turnip rape], Sinapis arvensis Brassica juncea, Brassica juncea var. juncea, Brassica juncea var. crispifolia, Brassica juncea var. foliosa, Brassica nigra, Brassica sinapioides, Melanosinapis communis [mustard], Brassica oleracea [fodder beet] or Arabidopsis thaliana; Bromeliaceae such as the genera Anana, Bromelia e.g. the species Anana comosus, Ananas ananas or Bromelia comosa [pineapple]; Caricaceae such as the genera Carica e.g. the species Carica papaya [papaya]; Cannabaceae such as the genera Cannabis e.g. the species Cannabis sative [hemp], Convolvulaceae such as the genera Ipomea, Convolvulus e.g. the species Ipomoea batatus, Ipomoea pandurata, Convolvulus batatas, Convolvulus tiliaceus, Ipomoea fastigiata, Ipomoea tiliacea, Ipomoea triloba or Convolvulus panduratus [sweet potato, Man of the Earth, wild potato], Chenopodiaceae such as the genera Beta, i.e. the species Beta vulgaris, Beta vulgaris var. altissima, Beta vulgaris var. Vulgaris, Beta maritima, Beta vulgaris var. perennis, Beta vulgaris var. conditiva or Beta vulgaris var. esculenta [sugar beet]; Cucurbitaceae such as the genera Cucubita e.g. the species Cucurbita maxima, Cucurbita mixta, Cucurbita pepo or Cucurbita moschata [pumpkin, squash]; Elaeagnaceae such as the genera Elaeagnus e.g. the species Olea europaea [olive]; Ericaceae such as the genera Kalmia e.g. the species Kalmia latifolia, Kalmia angustifolia, Kalmia microphylla, Kalmia polifolia, Kalmia occidentalis, Cistus chamaerhodendros or Kalmia lucida [American laurel, broad-leafed laurel, calico bush, spoon wood, sheep laurel, alpine laurel, bog laurel, western bog-laurel, swamp-laurel]; Euphorbiaceae such as the genera Manihot, Janipha, Jatropha, Ricinus e.g. the species Manihot utilissima, Janipha manihot, Jatropha manihot, Manihot aipil, Manihot dulcis, Manihot manihot, Manihot melanobasis, Manihot esculenta [manihot, arrowroot, tapioca, cassava] or Ricinus communis [castor bean, Castor Oil Bush, Castor Oil Plant, Palma Christi, Wonder Tree]; Fabaceae such as the genera Pisum, Albizia, Cathormion, Feuillea, Inga, Pithecolobium, Acacia, Mimosa, Medicajo, Glycine, Dolichos, Phaseolus, Soja e.g. the species Pisum sativum, Pisum arvense, Pisum humile [pea], Albizia berteriana, Albizia julibrissin, Albizia lebbeck, Acacia berteriana, Acacia littoralis, Albizia berteriana, Albizzia berteriana, Cathormion berteriana, Feuillea berteriana, Inga fragrans, Pithecellobium berterianum, Pithecellobium fragrans, Pithecolobium berterianum, Pseudalbizzia berteriana, Acacia julibrissin, Acacia nemu, Albizia nemu, Feuilleea julibrissin, Mimosa julibrissin, Mimosa speciosa, Sericanrda julibrissin, Acacia lebbeck, Acacia macrophylla, Albizia lebbek, Feuilleea lebbeck, Mimosa lebbeck, Mimosa speciosa [bastard logwood, silk tree, East Indian Walnut], Medicago sativa, Medicago falcata, Medicago varia [alfalfa], Glycine max [soybean], Dolichos soja, Glycine gracilis, Glycine hispida, Phaseolus max or Soja hispida; Geraniaceae such as the genera Pelargonium, Cocos, Oleum e.g. the species Cocos nucifera, Pelargonium grossularioides or Oleum cocois [coconut]; Gramineae such as the genera Saccharum e.g. the species Saccharum officinarum; Juglandaceae such as the genera Juglans, Wallia e.g. the species Juglans regia, Juglans ailanthifolia, Juglans sieboldiana, Juglans cinerea, Wallia cinerea, Juglans bixbyi, Juglans californica, Juglans hindsii, Juglans intermedia, Juglans jamaicensis, Juglans major, Juglans microcarpa, Juglans nigra or Wallia nigra [walnut, black walnut, common walnut, persian walnut, white walnut, butternut, black walnut]; Lauraceae such as the genera Persea, Laurus e.g. the species laurel Laurus nobilis [bay, laurel, bay laurel, sweet bay], Persea americana Persea americana, Persea gratissima or Persea persea [avocado]; Leguminosae such as the genera Arachis e.g. the species Arachis hypogaea [peanut]; Linaceae such as the genera Linum, Adenolinum e.g. the species Linum usitatissimum, Linum humile, Linum austriacum, Linum bienne, Linum angustifolium, Linum catharticum, Linum flavum, Linum grandiflorum, Adenolinum grandiflorum, Linum lewisii, Linum narbonense, Linum perenne, Linum perenne var. lewisii, Linum pratense or Linum trigynum [flax, linseed]; Lythrarieae such as the genera Punica e.g. the species Punica granatum [pomegranate]; Malvaceae such as the genera Gossypium e.g. the species Gossypium hirsutum, Gossypium arboreum, Gossypium barbadense, Gossypium herbaceum or Gossypium thurberi [cotton]; Musaceae such as the genera Musa e.g. the species Musa nana, Musa acuminate, Musa paradisiaca, Musa spp. [banana]; Onagraceae such as the genera Camissonia, Oenothera e.g. the species Oenothera biennis or Camissonia brevipes [primrose, evening primrose]; Palmae such as the genera Elacis e.g. the species Elaeis guineensis [oil plam]; Papaveraceae such as the genera Papaver e.g. the species Papaver orientale, Papaver rhoeas, Papaver dubium [poppy, oriental poppy, corn poppy, field poppy, shirley poppies, field poppy, longheaded poppy, long-pod poppy]; Pedaliaceae such as the genera Sesamum e.g. the species Sesamum indicum [sesame]; Piperaceae such as the genera Piper, Artanthe, Peperomia, Steffensia e.g. the species Piper aduncum, Piper amalago, Piper angustifolium, Piper auritum, Piper betel, Piper cubeba, Piper longum, Piper nigrum, Piper retrofractum, Artanthe adunca, Artanthe elongata, Peperomia elongata, Piper elongatum, Steffensia elongata. [Cayenne pepper, wild pepper]; Poaceae such as the genera Hordeum, Secale, Avena, Sorghum, Andropogon, Holcus, Panicum, Oryza, Zea, Triticum e.g. the species Hordeum vulgare, Hordeum jubaturn, Hordeum murinum, Hordeum secalinum, Hordeum distichon Hordeum aegiceras, Hordeum hexastichon, Hordeum hexastichum, Hordeum irregulare, Hordeum sativum, Hordeum secalinum [barley, pearl barley, foxtail barley, wall barley, meadow barley], Secale cereale [rye], Avena sativa, Avena fatua, Avena byzantina, Avena fatua var. sativa, Avena hybrida [oat], Sorghum bicolor, Sorghum halepense, Sorghum saccharatum, Sorghum vulgare, Andropogon drummondii, Holcus bicolor, Holcus sorghum, Sorghum aethiopicum, Sorghum arundinaceum, Sorghum caffrorum, Sorghum cernuum, Sorghum dochna, Sorghum drummondii, Sorghum durra, Sorghum guineense, Sorghum lanceolatum, Sorghum nervosum, Sorghum saccharatum, Sorghum subglabrescens, Sorghum verticilliflorum, Sorghum vulgare, Holcus halepensis, Sorghum miliaceum millet, Panicum militaceum [Sorghum, millet], Oryza sativa, Oryza latifolia [rice], Zea mays [corn, maize] Triticum aestivum, Triticum durum, Triticum turgidum, Triticum hybernum, Triticum macha, Triticum sativum or Triticum vulgare [wheat, bread wheat, common wheat], Proteaceae such as the genera Macadamia e.g. the species Macadamia intergrifolia [macadamia]; Rubiaceae such as the genera Coffea e.g. the species Cofea spp., Coffea arabica, Coffea canephora or Coffea liberica [coffee]; Scrophulariaceae such as the genera Verbascum e.g. the species Verbascum blattaria, Verbascum chaixii, Verbascum densiflorum, Verbascum lagurus, Verbascum longifolium, Verbascum lychnitis, Verbascum nigrum, Verbascum olympicum, Verbascum phlomoides, Verbascum phoenicum, Verbascum pulverulentum or Verbascum thapsus [mullein, white moth mullein, nettle-leaved mullein, dense-flowered mullein, silver mullein, long-leaved mullein, white mullein, dark mullein, greek mullein, orange mullein, purple mullein, hoary mullein, great mullein]; Solanaceae such as the genera Capsicum, Nicotiana, Solanum, Lycopersicon e.g. the species Capsicum annuum, Capsicum annuum var. glabriusculum, Capsicum frutescens [pepper], Capsicum annuum [paprika], Nicotiana tabacum, Nicotiana alata, Nicotiana attenuata, Nicotiana glauca, Nicotiana langsdorffii, Nicotiana obtusifolia, Nicotiana quadrivalvis, Nicotiana repanda, Nicotiana rustica, Nicotiana sylvestris [tobacco], Solanum tuberosum [potato], Solanum melongena [egg-plant] (Lycopersicon esculentum, Lycopersicon lycopersicum, Lycopersicon pyriforme, Solanum integrifolium or Solanum lycopersicum [tomato]; Sterculiaceae such as the genera Theobroma e.g. the species Theobroma cacao [cacao]; Theaceae such as the genera Camellia e.g. the species Camellia sinensis) [tea]; especially A. thaliana, maize, wheat, rye, oat, triticale, rice, barley, soybean, peanut, cotton, borage, sunflower, sugar cane, sugar beet, linseed, primrose, rapeseed, canola and turnip rape, manihot, pepper, sunflower, tagetes, solanaceous plant such as potato, tobacco, eggplant and tomato, Vicia species, pea, alfalfa, bushy plants such as coffee, cacao, tea, Salix species, trees such as oil palm, coconut, perennial grass, such as ryegrass and fescue, and forage crops, such as alfalfa and clover and from spruce, pine or fir for example.

More preferably said nucleic acid molecules can be isolated from S. cerevisiae, E. coli, Azotobacter vinelandii, Thermus thermophilus or Synechocystis sp. or plants, preferably Arabidopsis thaliana, Brassica napus, Glycine max, Zea mays, Gossypium or Oryza sativa.

The FCRPs of the present invention are preferably produced by recombinant DNA techniques. For example, a nucleic acid molecule encoding the protein is cloned into an expression vector, for example into a binary vector, the expression vector is introduced into a host cell, for example into cells of the Arabidopsis thaliana wild type NASC N906 or any other plant cell as described above and/or below and/or in the examples, and the FCRP is expressed in said host cell.

In an embodiment of the present invention, the respective FCRP is localized after expression as indicated in column 6 of Table II, e.g. non-targeted, mitochondrial or plastidic.

In an embodiment of the present invention the FCRP is preferably produced in an compartment of the cell, e.g. in an organelle, for example in plastids or mitochondria. Ways of introducing nucleic acids into organelles, for example into plastids or mitochondria, and producing proteins in this compartment are known to the person skilled in the art.

The respective nucleic acid sequence according to the invention mentioned above is advantageously functionally joined to a nucleic acid sequence encoding a transit peptide, in such a manner that a preprotein is translated, which is able to direct the polypeptide to the organelle such as to plastids or mitochondria. In another preferred embodiment the nucleic acids according to the invention mentioned above is advantageously functionally joined to a promotor region functional in plastids or mitochondria like for example the RNA operon promoter fused to the 5′UTR of the rbcL gene and in another preferred embodiment joined to a plastome sequences homologous to the integration sites. Example for useful integration sites are the trnV-rps12/7 (Skidar et al., Plant Cell Rep. 18, 20 (1998), and other reports), thr rbvL-aacD site (Svab et al., Proc. Natl. Acad. Sci. USA 90, 913 (1993)), the trnI-trnA site (De Cosa et al., Nat. Biotech. 19, 71 (2001)), the rps7-ndhB site (Hou et al., Transgenic Res. 12, 111 (2003)) and the ndhF-trnL site Zhang et al., Plant Physiol. 127, 131 (2001c)).

The nucleic acid sequence coding for the transit peptide is advantageously derived from a nucleic acid sequence encoding a protein finally resided in the plastids or mitochondria and is stemming from an organism selected from the group consisting of the genera Acetabularia, Arabidopsis, Brassica, Capsicum, Chlamydomonas, Cucurbita, Daucus, Dunaliella, Euglena, Flaveria, Glycine, Helianthus, Hordeum, Lactua, Lemna, Lolium, Lycopersion, Malus, Meticago, Mesembryanthemum, Nicotiana, Oenotherea, Oryza, Petunia, Phaseolus, Physcomitrella, Pinus, Pisum, Pyrus, Raphanus, Saccharum, Silene, Sinapis, Solanum, Spinacea, Stevia, Synechococcus,Triticum and Zea.

Preferably the plastidal transit peptide is derived from the nucleic acid sequence encoding a protein selected from the group consisting of ribulose bisphosphate carboxylase/oxygenase, 5′-enolpyruvyl-shikimate-3-phosphate synthase, acetolactate synthase, chloroplast ribosomal protein CS17, Cs protein, ferredoxin, plastocyanin, ribulose bisphosphate carboxylase activase, tryptophan synthase, acyl carrier protein, plastid chaperonin-60, cytochrome c552, 22-kDA heat shock protein, 33-kDa Oxygen-evolving enhancer protein 1, ATP synthase y subunit, ATP synthase σ subunit, chlorophyll-a/b-binding proteinII-1, Oxygen-evolving enhancer protein 2, Oxygen-evolving enhancer protein 3, photosystem I: P21, photosystem I: P28, photosystem I: P30, photosystem I: P35, photosystem I: P37, glycerol-3-phosphate acyltransferases, chlorophyll a/b binding protein, CAB2 protein, hydroxymethyl-bilane synthase, pyruvate-orthophosphate dikinase, CAB3 protein, plastid ferritin, ferritin, early light-inducible protein, glutamate-1-semialdehyde aminotransferase, protochlorophyllide reductase, starch-granule-bound amylase synthase, light-harvesting chlorophyll a/b-binding protein of photosystem II, major pollen allergen Lol p 5a, plastid CIpB ATP-dependent protease, superoxide dismutase, ferredoxin NADP oxidoreductase, 28-kDa ribonucleoprotein, 31-kDa ribonucleoprotein, 33-kDa ribonucleoprotein, acetolactate synthase, ATP synthase CFO subunit 1, ATP synthase CFO subunit 2, ATP synthase CFO subunit 3, ATP synthase CFO subunit 4, cytochrome f, ADP-glucose pyrophosphorylase, glutamine synthase, glutamine synthase 2, carbonic anhydrase, GapA protein, heat-shock-protein hsp21, phosphate translocator, plastid CIpA ATP-dependent protease, plastid ribosomal protein CL24, plastid ribosomal protein CL9, plastid ribosomal protein PsCL18, plastid ribosomal protein PsCL25, DAHP synthase, starch phosphorylase, root acyl carrier protein II, betaine-aldehyde dehydrogenase, GapB protein, glutamine synthetase 2, phosphoribulokinase, nitrite reductase, ribosomal protein L12, ribosomal protein L13, ribosomal protein L21, ribosomal protein L35, ribosomal protein L40, triose phosphate-3-phosphoglyerate-phosphate translocator, ferredoxin-dependent glutamate synthase, glyceraldehyde-3-phosphate dehydrogenase, NADP-dependent malic enzyme and NADP-malate dehydrogenase.

In an embodiment the plastome sequences are preferential derived from the plastome of the target organisms themselves and are advantegously derived from one of the following intergration sites: trnV-rps12/7 (Skidar et al., Plant Cell Rep. 18, 20 (1998) and other reports), rbvL-aacD (Svab et al., Proc. Natl. Acad. Sci. USA 90, 913 (1993)), trnl-trnA (De Cosa et al., Nat. Biotech. 19, 71 (2001)), rps7-ndhB (Hou et al., Transgenic Res. 12, 111 (2003)) or ndhF-trnL site Zhang et al., Plant Physiol. 127, 131 (2001c)).

Advantageously the mitochondrial transit peptide is derived from the nucleic acid sequence encoding a protein selected from the group consisting of

22 kDA heat shock protein; 70 kDA heat shock protein; 83 kDA heat shock protein; 40S ribosomal protein S19; 50S ribosomal protein L15; ribosomal protein L29; 22 kDA PSST protein of complex I; 2-oxoacid dehydrogenase family protein; 2-oxoglutarate/malate translocator; 3-methyl-2-oxobutanoate hydroxymethyltransferase; 3-Methylcrotonyl-coenzyme A carboxylase (MCCase); 7,8-Dihydropteroate synthase (DHPS)/6-hydroxymethyl-7,8-dihydropterine pyrophosphokinase (HPPK); aconitate hydratase; acyl carrier protein (ACP); ADP/ATP translocase; alanyl-tRNA synthetase; alcohol dehydrogenase (ADH); alternative oxidase (AOX); aminoacyl-tRNA ligase; asparate aminotransferase; ATP synthase alpha subunit; ATP synthase beta subunit; ATP synthase delta subunit; ATP synthase epsilon subunit; ATP synthase gamma subunit; ATP-dependent Clp protease-proteolytic subunit; Chaperonin 60-CPN60; Chaperonin 60 (2)-CPN60-2; Chaperonin 60(1)-CPN60-1; citrate synthase; cytochrome b-c1 complex subunit Rieske FeS Protein; cytochrome c reductase-processing peptidase subunit II; dihydrolipoamide S-acetyltransferase; farnesyl-diphosphate synthase 1; formate dehydrogenase; fumarate hydratase; gamma carbonic anhydrase protein (gammaCA); gamma carbonic anhydrase-like protein 1 (gammaCAL1); gamma carbonic anhydrase-like protein 2 (gammaCAL2); gamma-aminobutyric acid transaminase (GABA-T); glutathione reductase (GR); glycine decarboxylase subunit H; glycine decarboxylase subunit L; glycine decarboxylase subunit P; glycine decarboxylase subunit T; isovaleryl-CoA dehydrogenase (IVD); lipoamide dehydrogenase; malate oxidoreductase; manganese superoxide dismutase (Mn)SOD; methylmalonate-semialdehyde dehydrogenase; mitochondrial-processing peptidase beta subunit (MPP); mitochondrial-processing peptidase subunit alpha (MPP); monodehydroascorbate reductase (MDHAR); NAD dependent isocitrate dehydrogenase; NAD dependent malate dehydrogenase; NAD-dependent malic enzyme; NAD-dependent malic enzyme 59 kDa isoform; NAD-dependent malic enzyme 62 kDa isoform; NAD-dependent malic enzyme 65 kDa isoform; NADH ubiquinone oxidoreductase 29 kDa subunit; NADH-ubiquinone oxidoreductase 18 kDa subunit; NADH-ubiquinone oxidoreductase 20 kDa subunit; NADH-ubiquinone oxidoreductase 23 kDa subunit; NADHubiquinone oxidoreductase 75 kDa subunit; NADP dependent isocitrate dehydrogenase; NADP dependent malate dehydrogenase; nucleoside diphosphate kinase; nucleoside diphosphate kinase III; o-acetylserine (thiol) lyase (OAS-TL); propionyl-CoA carboxylase; protoporphyrinogen IX oxidase; pyruvate dehydrogenase E1 component subunit alpha; serine acetyltransferase (SAT); serine hydroxymethyltransferase; succinate dehydrogenase (SDH); succinic semialdehyde dehydrogenase (SSADH); succinyl-CoA ligase (GDP-forming) alpha-chain; succinyl-CoA ligase [GDP-forming] subunit beta; thiosulfate sulfurtransferase; threonyl-tRNA synthetase; trans-2-enoyl-CoA reductase; translocase inner membrane (TIM); translocase outer membrane (TOM); tRNA synthetase class I and ubiquinol cytochrome C oxidoreductase complex.

Further transit peptids and details thereto have been described above.

In another embodiment of the present invention the FCRP is produced without further targeting signal (e.g. as mentioned herein), e.g. in the cytoplasm of the cell. Ways of producing proteins without artificial targeting are known to the person skilled in the art.

The nucleic acid sequences used in the process are advantageously introduced in a nucleic acid construct, preferably an expression cassette, which makes possible the expression of the nucleic acid molecules in a non-human organism, advantageously a plant or a microorganism such as an algae, advantegously non-targeted or in an organelle, like plastids or mitochondria , respectively, of those non-human organisms.

Accordingly, the invention also relates to a nucleic acid construct, preferably to an expression construct, comprising the nucleic acid molecule of the present invention functionally linked to one or more regulatory elements or signals.

As described herein, the nucleic acid construct can also comprise further genes, which are to be introduced into the non-human organisms or cells. It is possible and advantageous to introduce into, and express in, the host organisms regulatory genes such as genes for inductors, repressors or enzymes, which, owing to their biological or enzymatic activity, engage in the regulation of one or more genes of the respective biosynthetic pathway. These genes can be of heterologous or homologous origin. Moreover, further biosynthesis genes may advantageously be present, or else these genes may be located on one or more further nucleic acid constructs. Genes, which are advantageously employed as further biosynthesis genes are genes of the fatty acid metabolism, amino acid metabolism, of glycolysis, of the tricarboxylic acid metabolism or their combinations. As described herein, regulator sequences or factors can have a positive effect on preferably the gene expression of the genes introduced, thus increasing it. Thus, an enhancement of the regulator elements may advantageously take place at the transcriptional level by using strong transcription signals such as promoters and/or enhancers. In addition, however, an enhancement of translation is also possible, for example by increasing mRNA stability or by inserting a translation enhancer sequence.

In principle, the nucleic acid construct can comprise the herein described regulator sequences and further sequences relevant for the expression of the comprised genes. Thus, the nucleic acid construct of the invention can be used as expression cassette and thus can be used directly for introduction into the plant, or else they may be introduced into a vector. Accordingly in one embodiment the nucleic acid construct is an expression cassette comprising a microorganism promoter or a microorganism terminator or both, beneath the nucleic acid molecule according to the invention. In another embodiment the expression cassette encompasses a plant promoter or a plant terminator or both, beneath the nucleic acid molecule according to the invention. In another embodiment the expression cassette encompasses sequences for transcription by organelle RNA polymerases, beneath the nucleic acid molecule according to the invention. In another embodiment an expression cassette encompasses a nucleic acid molecule encoding for a transit peptide, beneath the gene according to the invention.

In a preferred embodiment a nucleic acid construct, for example an expression cassette, comprises upstream, i.e. at the 5′ end of the encoding sequence, a promoter and downstream, i.e. at the 3′ end, a polyadenylation signal and optionally other regulatory elements which are operably linked to the intervening encoding sequence with one of the nucleic acids as depicted in the respective line in Table I, application no. 1, column 5 or 8, preferably the coding region thereof, or a homolog or a fragment thereof. By an operable linkage is meant the sequential arrangement of promoter, encoding sequence, terminator, optionally other regulatory elements and optionally targeting sequences in such a way that each of the regulatory elements/targeting sequence can fulfill its function in the expression of the encoding sequence in due manner. In one embodiment the sequences preferred for operable linkage are targeting sequences for ensuring subcellular localization in organelles, like plastids or mitochondria. However, targeting sequences for ensuring subcellular localization in the endoplasmic reticulum (=ER), in the nucleus, in oil corpuscles or other compartments may also be employed as well as translation promoters such as the 5′ lead sequence in tobacco mosaic virus (Gallie et al., Nucl. Acids Res. 15, 8693 (1987)).

As an example, a nucleic acid construct, like an expression cassette may, for example, contain a constitutive promoter or a tissue-specific promoter (preferably the USP or napin promoter), the gene to be expressed and a ER retention signal. For the ER retention signal the KDEL amino acid sequence (lysine, aspartic acid, glutamic acid, leucine) or the KKX amino acid sequence (lysine-lysine-X-stop, wherein X means every other known amino acid) is preferably employed.

In an embodiment, the invention relates to an expression cassette, comprising
1) a promoter, prefereably a promoter selected from the group consisting of Big35S, PCUbi, Super and USP;
2) a nucleic acid molecule selected from the group consisting of
   (a) a nucleic acid molecule encoding a polypeptide shown in Table II, application no. 1, column 5 or 8, preferably shown in Table II A, application no. 1, column 5, or in Table II A, application no. 1, column 8, or in Table II B, application no. 1, column 8, or a homolog or a fragment thereof;
   (b) a nucleic acid molecule shown in Table I, application no. 1, column 5 or 8, preferably shown in Table I A, application no. 1, column 5, or in Table I A, application no. 1, column 8, or in Table I B, application no. 1, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 1, preferably shown in Table II A, application no. 1, column 5, or in Table II A, application no. 1, column 8, or in Table II B, application no. 1, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 1, preferably shown in Table I A, application no. 1, column 5, or in Table I A, application no. 1, column 8,or in Table I B, application no. 1, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridisation conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 1;
   (i) a nucleic acid molecule encoding a polypeptide having the activity represented by a respective protein as depicted in column 5 of Table II, application no. 1;
   (j) a nucleic acid molecule comprising a polynucleotide which is obtained by amplifying a cDNA library or a genomic library using the primers in Table III, application no. 1, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto.
      and, optionally
3) nucleic acid molecule encoding a transit peptide, preferably encoding a plastidal transit peptide or a mitochondrial transit peptide;
   which are operable linked.
   (All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 1.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 1 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 1, in the same line as of the respective nucleic acid molecule according to 2) “plastidic” is depicted, or encoding a mitochondrial transit peptide, especially in case in column 6 of Table I, application no. 1, in the same line as of the respective nucleic acid molecule according to 2) “mitochondrial” is depicted.

In another embodiment thereof the expression cassette does not comprise 3) a nucleic acid molecule encoding a transit peptide, especially in case in column 6 of Table I, application no. 1, in the same line as of the respective nucleic acid molecule according to 2) “non-targeted” is depicted.

In a further embodiment, the invention relates to an expression cassette, comprising
1) a promoter, prefereably a promoter selected from the group consisting of Big35S, PCUbi, Super and USP;
2) a nucleic acid molecule selected from the group consisting of
   (a) a nucleic acid molecule encoding a polypeptide shown in Table II, application no. 1, column 5 or 8, preferably shown in Table II A, application no. 1, column 5, or in Table II A, application no. 1, column 8, or in Table II B, application no. 1, column 8, or a homolog or a fragment thereof;
   (b) a nucleic acid molecule shown in Table I, application no. 1, column 5 or 8, preferably shown in Table I A, application no. 1, column 5, or in Table I A, application no. 1, column 8, or in Table I B, application no. 1, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 1, preferably shown in Table II A, application no. 1, column 5, or in Table II A, application no. 1, column 8, or in Table II B, application no. 1, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 1, preferably shown in Table I A, application no. 1, column 5, or in Table I A, application no. 1, column 8, or in Table I B, application no. 1, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridisation conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 1;
   (i) a nucleic acid molecule encoding a polypeptide having the activity represented by a respective protein as depicted in column 5 of Table II, application no. 1;
   (j) a nucleic acid molecule comprising a polynucleotide which is obtained by amplifying a cDNA library or a genomic library using the primers in Table III, application no. 1, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, optionally
3) nucleic acid molecule encoding a transit peptide, preferably encoding a plastidal transit peptide or a mitochondrial transit peptide;
   which are operable linked and
   whereby, preferably, the nucleic acid molecule according to 2 (a), (b), (c), (d), (e), (f), (g), (h), (i), (j) or (k) distinguishes over the sequence depicted in Table I A and/or I B, application no. 1, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention according to 2) does not consist of the sequence shown in Table I A and/or I B, application no. 1, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 1, column 5 or 8, or the coding region thereof, respectively. In a further embodiment the nucleic acid molecule does not encode the polypeptide sequence shown in Table II A and/or II B, application no. 1, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 1, column 5 or 8 and does not encode a protein of the sequence shown in Table II A and/or II B, application no. 1, column 5 or 8. Accordingly, in one embodiment, the protein encoded by a sequence of a nucleic acid according to 2 (a), (b), (c), (d), (e), (f), (g), (h), (i), (j) or (k) does not consist of the sequence shown in Table I A and/or I B, application no. 1, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 1, column 5 or 8 but less than 100%, 99.999%, 99.99%, 99.9%, 99%, 98%, 97%, 96% or 95% identical to the sequence shown in Table II A and/or II B, application no. 1, column 5 or 8.
   (All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 1.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 1 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 1, in the same line as of the respective nucleic acid molecule according to 2) “plastidic” is depicted, or encoding a mitochondrial transit peptide, especially in case in column 6 of Table I, application no. 1, in the same line as of the respective nucleic acid molecule according to 2) “mitochondrial” is depicted.

In another embodiment thereof the expression cassette does not comprise 3) a nucleic acid molecule encoding a transit peptide, especially in case in column 6 of Table I, application no. 1, in the same line as of the respective nucleic acid molecule according to 2) “non-targeted” is depicted.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 15175, or a homolog or fragment thereof, or a homolog thereof being depicted in Table I, application no. 1, column 8, in the same line as SEQ ID NO. 15175, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 15175 is depicted, plastidic is mentioned, or a mitochondrial transit petide in case in Table I, application no. 1, in column 6 in the same line as SEQ ID NO. 15175 is depicted, mitochondrial is mentioned; which is operable linke, too.

In another embodiment thereof the expression cassette does not comprise an additional transit peptide in case in Table I, application no. 1, in column 6 in the same line as SEQ ID NO. 15175 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 9167, or a homolog or fragment thereof, or a homolog thereof being depicted in Table I, application no. 1, column 8, in the same line as SEQ ID NO. 9167, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 9167 is depicted, plastidic is mentioned, or a mitochondrial transit petide in case in Table I, application no. 1, in column 6 in the same line as SEQ ID NO. 9167 is depicted, mitochondrial is mentioned; which is operable linke, too.

In another embodiment thereof the expression cassette does not comprise an additional transit peptide in case in Table I, application no. 1, in column 6 in the same line as SEQ ID NO. 9167 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 9244, or a homolog or fragment thereof, or a homolog thereof being depicted in Table I, application no. 1, column 8, in the same line as SEQ ID NO. 9244, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 9244 is depicted, plastidic is mentioned, or a mitochondrial transit petide in case in Table I, application no. 1, in column 6 in the same line as SEQ ID NO. 9244 is depicted, mitochondrial is mentioned; which is operable linke, too.

In another embodiment thereof the expression cassette does not comprise an additional transit peptide in case in Table I, application no. 1, in column 6 in the same line as SEQ ID NO. 9244 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 14302, or a homolog or fragment thereof, or a homolog thereof being depicted in Table I, application no. 1, column 8, in the same line as SEQ ID NO. 14302, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 14302 is depicted, plastidic is mentioned, or a mitochondrial transit petide in case in Table I, application no. 1, in column 6 in the same line as SEQ ID NO. 14302 is depicted, mitochondrial is mentioned; which is operable linke, too.

In another embodiment thereof the expression cassette does not comprise an additional transit peptide in case in Table I, application no. 1, in column 6 in the same line as SEQ ID NO. 14302 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 14706, or a homolog or fragment thereof, or a homolog thereof being depicted in Table I, application no. 1, column 8, in the same line as SEQ ID NO. 14706, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 14706 is depicted, plastidic is mentioned, or a mitochondrial transit petide in case in Table I, application no. 1, in column 6 in the same line as SEQ ID NO. 14706 is depicted, mitochondrial is mentioned; which is operable linke, too.

In another embodiment thereof the expression cassette does not comprise an additional transit peptide in case in Table I, application no. 1, in column 6 in the same line as SEQ ID NO. 14706 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 14715, or a homolog or fragment thereof, or a homolog thereof being depicted in Table I, application no. 1, column 8, in the same line as SEQ ID NO. 14715, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 14715 is depicted, plastidic is mentioned, or a mitochondrial transit petide in case in Table I, application no. 1, in column 6 in the same line as SEQ ID NO. 14715 is depicted, mitochondrial is mentioned; which is operable linke, too.

In another embodiment thereof the expression cassette does not comprise an additional transit peptide in case in Table I, application no. 1, in column 6 in the same line as SEQ ID NO. 14715 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 14769, or a homolog or fragment thereof, or a homolog thereof being depicted in Table I, application no. 1, column 8, in the same line as SEQ ID NO. 14769, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 14769 is depicted, plastidic is mentioned, or a mitochondrial transit petide in case in Table I, application no. 1, in column 6 in the same line as SEQ ID NO. 14769 is depicted, mitochondrial is mentioned; which is operable linke, too.

In another embodiment thereof the expression cassette does not comprise an additional transit peptide in case in Table I, application no. 1, in column 6 in the same line as SEQ ID NO. 14769 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 14821, or a homolog or fragment thereof, or a homolog thereof being depicted in Table I, application no. 1, column 8, in the same line as SEQ ID NO. 14821, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 14821 is depicted, plastidic is mentioned, or a mitochondrial transit petide in case in Table I, application no. 1, in column 6 in the same line as SEQ ID NO. 14821 is depicted, mitochondrial is mentioned; which is operable linke, too.

In another embodiment thereof the expression cassette does not comprise an additional transit peptide in case in Table I, application no. 1, in column 6 in the same line as SEQ ID NO. 14821 is depicted, non-targeted is mentioned.

In an embodiment the present invention relates to an expresssion cassette comprising a) a promoter, prefereably a promoter selected from the group consisting of Big35S, PCUbi, Super and USP, in particular USP; and b) the nucleic acid SEQ ID NO. 10252, or a homolog or fragment thereof, or a homolog thereof being depicted in Table I, application no. 1, column 8, in the same line as SEQ ID NO. 10252, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 10252 is depicted, plastidic is mentioned, or a mitochondrial transit petide in case in Table I, application no. 1, in column 6 in the same line as SEQ ID NO. 10252 is depicted, mitochondrial is mentioned; which is operable linke, too.

In another embodiment thereof the expression cassette does not comprise an additional transit peptide in case in Table I, application no. 1, in column 6 in the same line as SEQ ID NO. 10252 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 14885, or a homolog or fragment thereof, or a homolog thereof being depicted in Table I, application no. 1, column 8, in the same line as SEQ ID NO. 14885, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 14885 is depicted, plastidic is mentioned, or a mitochondrial transit petide in case in Table I, application no. 1, in column 6 in the same line as SEQ ID NO. 14885 is depicted, mitochondrial is mentioned; which is operable linke, too.

In another embodiment thereof the expression cassette does not comprise an additional transit peptide in case in Table I, application no. 1, in column 6 in the same line as SEQ ID NO. 14885 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 8920, or a homolog or fragment thereof, or a homolog thereof being depicted in Table I, application no. 1, column 8, in the same line as SEQ ID NO. 8920, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 8920 is depicted, plastidic is mentioned, or a mitochondrial transit petide in case in Table I, application no. 1, in column 6 in the same line as SEQ ID NO. 8920 is depicted, mitochondrial is mentioned; which is operable linke, too.

In another embodiment thereof the expression cassette does not comprise an additional transit peptide in case in Table I, application no. 1, in column 6 in the same line as SEQ ID NO. 8920 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 15179, or a homolog or fragment thereof, or a homolog thereof being depicted in Table I, application no. 1, column 8, in the same line as SEQ ID NO. 15179, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 15179 is depicted, plastidic is mentioned, or a mitochondrial transit petide in case in Table I, application no. 1, in column 6 in the same line as SEQ ID NO. 15179 is depicted, mitochondrial is mentioned; which is operable linke, too.

In another embodiment thereof the expression cassette does not comprise an additional transit peptide in case in Table I, application no. 1, in column 6 in the same line as SEQ ID NO. 15179 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 15183, or a homolog or fragment thereof, or a homolog thereof being depicted in Table I, application no. 1, column 8, in the same line as SEQ ID NO. 15183, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 15183 is depicted, plastidic is mentioned, or a mitochondrial transit petide in case in Table I, application no. 1, in column 6 in the same line as SEQ ID NO. 15183 is depicted, mitochondrial is mentioned; which is operable linke, too.

In another embodiment thereof the expression cassette does not comprise an additional transit peptide in case in Table I, application no. 1, in column 6 in the same line as SEQ ID NO. 15183 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 9333, or a homolog or fragment thereof, or a homolog thereof being depicted in Table I, application no. 1, column 8, in the same line as SEQ ID NO. 9333, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 9333 is depicted, plastidic is mentioned, or a mitochondrial transit petide in case in Table I, application no. 1, in column 6 in the same line as SEQ ID NO. 9333 is depicted, mitochondrial is mentioned; which is operable linke, too.

In another embodiment thereof the expression cassette does not comprise an additional transit peptide in case in Table I, application no. 1, in column 6 in the same line as SEQ ID NO. 9333 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 9470, or a homolog or fragment thereof, or a homolog thereof being depicted in Table I, application no. 1, column 8, in the same line as SEQ ID NO. 9470, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 9470 is depicted, plastidic is mentioned, or a mitochondrial transit petide in case in Table I, application no. 1, in column 6 in the same line as SEQ ID NO. 9470 is depicted, mitochondrial is mentioned; which is operable linke, too.

In another embodiment thereof the expression cassette does not comprise an additional transit peptide in case in Table I, application no. 1, in column 6 in the same line as SEQ ID NO. 9470 is depicted, non-targeted is mentioned.

In an embodiment the present invention relates to an expresssion cassette comprising a) a promoter, prefereably a promoter selected from the group consisting of Big35S, PCUbi, Super and USP, in particular USP; and b) the nucleic acid SEQ ID NO. 9492, or a homolog or fragment thereof, or a homolog thereof being depicted in Table I, application no. 1, column 8, in the same line as SEQ ID NO. 9492, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 9492 is depicted, plastidic is mentioned, or a mitochondrial transit petide in case in Table I, application no. 1, in column 6 in the same line as SEQ ID NO. 9492 is depicted, mitochondrial is mentioned; which is operable linke, too.

In another embodiment thereof the expression cassette does not comprise an additional transit peptide in case in Table I, application no. 1, in column 6 in the same line as SEQ ID NO. 9492 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 10104, or a homolog or fragment thereof, or a homolog thereof being depicted in Table I, application no. 1, column 8, in the same line as SEQ ID NO. 10104, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 10104 is depicted, plastidic is mentioned, or a mitochondrial transit petide in case in Table I, application no. 1, in column 6 in the same line as SEQ ID NO. 10104 is depicted, mitochondrial is mentioned; which is operable linke, too.

In another embodiment thereof the expression cassette does not comprise an additional transit peptide in case in Table I, application no. 1, in column 6 in the same line as SEQ ID NO. 10104 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 6075, or a homolog or fragment thereof, or a homolog thereof being depicted in Table I, application no. 1, column 8, in the same line as SEQ ID NO. 6075, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 6075 is depicted, plastidic is mentioned, or a mitochondrial transit petide in case in Table I, application no. 1, in column 6 in the same line as SEQ ID NO. 6075 is depicted, mitochondrial is mentioned; which is operable linke, too.

In another embodiment thereof the expression cassette does not comprise an additional transit peptide in case in Table I, application no. 1, in column 6 in the same line as SEQ ID NO. 6075 is depicted, non-targeted is mentioned.

In an embodiment the present invention relates to an expresssion cassette comprising a) a promoter, prefereably a promoter selected from the group consisting of Big35S, PCUbi, Super and USP, in particular Big35S; and b) the nucleic acid SEQ ID NO. 14843, or a homolog or fragment thereof, or a homolog thereof being depicted in Table I, application no. 1, column 8, in the same line as SEQ ID NO. 14843, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 14843 is depicted, plastidic is mentioned, or a mitochondrial transit petide in case in Table I, application no. 1, in column 6 in the same line as SEQ ID NO. 14843 is depicted, mitochondrial is mentioned; which is operable linke, too.

In another embodiment thereof the expression cassette does not comprise an additional transit peptide in case in Table I, application no. 1, in column 6 in the same line as SEQ ID NO. 14843 is depicted, non-targeted is mentioned.

In an embodiment the present invention relates to an expresssion cassette comprising a) a promoter, prefereably a promoter selected from the group consisting of Big35S, PCUbi, Super and USP, in particular USP; and b) the nucleic acid SEQ ID NO. 7730, or a homolog or fragment thereof, or a homolog thereof being depicted in Table I, application no. 1, column 8, in the same line as SEQ ID NO. 7730, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 7730 is depicted, plastidic is mentioned, or a mitochondrial transit petide in case in Table I, application no. 1, in column 6 in the same line as

SEQ ID NO. 7730 is depicted, mitochondrial is mentioned; which is operable linke, too.

In another embodiment thereof the expression cassette does not comprise an additional transit peptide in case in Table I, application no. 1, in column 6 in the same line as SEQ ID NO. 7730 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 6268, or a homolog or fragment thereof, or a homolog thereof being depicted in Table I, application no. 1, column 8, in the same line as SEQ ID NO. 6268, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 6268 is depicted, plastidic is mentioned, or a mitochondrial transit petide in case in Table I, application no. 1, in column 6 in the same line as SEQ ID NO. 6268 is depicted, mitochondrial is mentioned; which is operable linke, too.

In another embodiment thereof the expression cassette does not comprise an additional transit peptide in case in Table I, application no. 1, in column 6 in the same line as SEQ ID NO. 6268 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 6510, or a homolog or fragment thereof, or a homolog thereof being depicted in Table I, application no. 1, column 8, in the same line as SEQ ID NO. 6510, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 6510 is depicted, plastidic is mentioned, or a mitochondrial transit petide in case in Table I, application no. 1, in column 6 in the same line as SEQ ID NO. 6510 is depicted, mitochondrial is mentioned; which is operable linke, too.

In another embodiment thereof the expression cassette does not comprise an additional transit peptide in case in Table I, application no. 1, in column 6 in the same line as SEQ ID NO. 6510 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 6674, or a homolog or fragment thereof, or a homolog thereof being depicted in Table I, application no. 1, column 8, in the same line as SEQ ID NO. 6674, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 6674 is depicted, plastidic is mentioned, or a mitochondrial transit petide in case in Table I, application no. 1, in column 6 in the same line as SEQ ID NO. 6674 is depicted, mitochondrial is mentioned; which is operable linke, too.

In another embodiment thereof the expression cassette does not comprise an additional transit peptide in case in Table I, application no. 1, in column 6 in the same line as SEQ ID NO. 6674 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 6810, or a homolog or fragment thereof, or a homolog thereof being depicted in Table I, application no. 1, column 8, in the same line as SEQ ID NO. 6810, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 6810 is depicted, plastidic is mentioned, or a mitochondrial transit petide in case in Table I, application no. 1, in column 6 in the same line as SEQ ID NO. 6810 is depicted, mitochondrial is mentioned; which is operable linke, too.

In another embodiment thereof the expression cassette does not comprise an additional transit peptide in case in Table I, application no. 1, in column 6 in the same line as SEQ ID NO. 6810 is depicted, non-targeted is mentioned.

In an embodiment the present invention relates to an expresssion cassette comprising a) a promoter, prefereably a promoter selected from the group consisting of Big35S, PCUbi, Super and USP, in particular USP; and b) the nucleic acid SEQ ID NO. 6818, or a homolog or fragment thereof, or a homolog thereof being depicted in Table I, application no. 1, column 8, in the same line as SEQ ID NO. 6818, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 6818 is depicted, plastidic is mentioned, or a mitochondrial transit petide in case in Table I, application no. 1, in column 6 in the same line as SEQ ID NO. 6818 is depicted, mitochondrial is mentioned; which is operable linke, too.

In another embodiment thereof the expression cassette does not comprise an additional transit peptide in case in Table I, application no. 1, in column 6 in the same line as SEQ ID NO. 6818 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 7081, or a homolog or fragment thereof, or a homolog thereof being depicted in Table I, application no. 1, column 8, in the same line as SEQ ID NO. 7081, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 7081 is depicted, plastidic is mentioned, or a mitochondrial transit petide in case in Table I, application no. 1, in column 6 in the same line as SEQ ID NO. 7081 is depicted, mitochondrial is mentioned; which is operable linke, too.

In another embodiment thereof the expression cassette does not comprise an additional transit peptide in case in Table I, application no. 1, in column 6 in the same line as SEQ ID NO. 7081 is depicted, non-targeted is mentioned.

In an embodiment the present invention relates to an expresssion cassette comprising a) a promoter, prefereably a promoter selected from the group consisting of Big35S, PCUbi, Super and USP, in particular USP; and b) the nucleic acid SEQ ID NO. 7269, or a homolog or fragment thereof, or a homolog thereof being depicted in Table I, application no. 1, column 8, in the same line as SEQ ID NO. 7269, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 7269 is depicted, plastidic is mentioned, or a mitochondrial transit petide in case in Table I, application no. 1, in column 6 in the same line as SEQ ID NO. 7269 is depicted, mitochondrial is mentioned; which is operable linke, too.

In another embodiment thereof the expression cassette does not comprise an additional transit peptide in case in Table I, application no. 1, in column 6 in the same line as SEQ ID NO. 7269 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 9156, or a homolog or fragment thereof, or a homolog thereof being depicted in Table I, application no. 1, column 8, in the same line as SEQ ID NO. 9156, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 9156 is depicted, plastidic is mentioned, or a mitochondrial transit petide in case in Table I, application no. 1, in column 6 in the same line as SEQ ID NO. 9156 is depicted, mitochondrial is mentioned; which is operable linke, too.

In another embodiment thereof the expression cassette does not comprise an additional transit peptide in case in Table I, application no. 1, in column 6 in the same line as SEQ ID NO. 9156 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 7686, or a homolog or fragment thereof, or a homolog thereof being depicted in Table I, application no. 1, column 8, in the same line as SEQ ID NO. 7686, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 7686 is depicted, plastidic is mentioned, or a mitochondrial transit petide in case in Table I, application no. 1, in column 6 in the same line as SEQ ID NO. 7686 is depicted, mitochondrial is mentioned; which is operable linke, too.

In another embodiment thereof the expression cassette does not comprise an additional transit peptide in case in Table I, application no. 1, in column 6 in the same line as SEQ ID NO. 7686 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 8937, or a homolog or fragment thereof, or a homolog thereof being depicted in Table I, application no. 1, column 8, in the same line as SEQ ID NO. 8937, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 8937 is depicted, plastidic is mentioned, or a mitochondrial transit petide in case in Table I, application no. 1, in column 6 in the same line as SEQ ID NO. 8937 is depicted, mitochondrial is mentioned; which is operable linke, too.

In another embodiment thereof the expression cassette does not comprise an additional transit peptide in case in Table I, application no. 1, in column 6 in the same line as SEQ ID NO. 8937 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 7917, or a homolog or fragment thereof, or a homolog thereof being depicted in Table I, application no. 1, column 8, in the same line as SEQ ID NO. 7917, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 7917 is depicted, plastidic is mentioned, or a mitochondrial transit petide in case in Table I, application no. 1, in column 6 in the same line as SEQ ID NO. 7917 is depicted, mitochondrial is mentioned; which is operable linke, too.

In another embodiment thereof the expression cassette does not comprise an additional transit peptide in case in Table I, application no. 1, in column 6 in the same line as SEQ ID NO. 7917 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 7941, or a homolog or fragment thereof, or a homolog thereof being depicted in Table I, application no. 1, column 8, in the same line as SEQ ID NO. 7941, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 7941 is depicted, plastidic is mentioned, or a mitochondrial transit petide in case in Table I, application no. 1, in column 6 in the same line as SEQ ID NO. 7941 is depicted, mitochondrial is mentioned; which is operable linke, too.

In another embodiment thereof the expression cassette does not comprise an additional transit peptide in case in Table I, application no. 1, in column 6 in the same line as SEQ ID NO. 7941 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 7947, or a homolog or fragment thereof, or a homolog thereof being depicted in Table I, application no. 1, column 8, in the same line as SEQ ID NO. 7947, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 7947 is depicted, plastidic is mentioned, or a mitochondrial transit petide in case in Table I, application no. 1, in column 6 in the same line as SEQ ID NO. 7947 is depicted, mitochondrial is mentioned; which is operable linke, too.

In another embodiment thereof the expression cassette does not comprise an additional transit peptide in case in Table I, application no. 1, in column 6 in the same line as SEQ ID NO. 7947 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 7992, or a homolog or fragment thereof, or a homolog thereof being depicted in Table I, application no. 1, column 8, in the same line as SEQ ID NO. 7992, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 7992 is depicted, plastidic is mentioned, or a mitochondrial transit petide in case in Table I, application no. 1, in column 6 in the same line as SEQ ID NO. 7992 is depicted, mitochondrial is mentioned; which is operable linke, too.

In another embodiment thereof the expression cassette does not comprise an additional transit peptide in case in Table I, application no. 1, in column 6 in the same line as SEQ ID NO. 7992 is depicted, non-targeted is mentioned.

In an embodiment the present invention relates to an expresssion cassette comprising a) a promoter, prefereably a promoter selected from the group consisting of Big35S, PCUbi, Super and USP, in particular USP; and b) the nucleic acid SEQ ID NO. 8033, or a homolog or fragment thereof, or a homolog thereof being depicted in Table I, application no. 1, column 8, in the same line as SEQ ID NO. 8033, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 8033 is depicted, plastidic is mentioned, or a mitochondrial transit petide in case in Table I, application no. 1, in column 6 in the same line as SEQ ID NO. 8033 is depicted, mitochondrial is mentioned; which is operable linke, too.

In another embodiment thereof the expression cassette does not comprise an additional transit peptide in case in Table I, application no. 1, in column 6 in the same line as SEQ ID NO. 8033 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 8316, or a homolog or fragment thereof, or a homolog thereof being depicted in Table I, application no. 1, column 8, in the same line as SEQ ID NO. 8316, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 8316 is depicted, plastidic is mentioned, or a mitochondrial transit petide in case in Table I, application no. 1, in column 6 in the same line as SEQ ID NO. 8316 is depicted, mitochondrial is mentioned; which is operable linke, too.

In another embodiment thereof the expression cassette does not comprise an additional transit peptide in case in Table I, application no. 1, in column 6 in the same line as SEQ ID NO. 8316 is depicted, non-targeted is mentioned.

In an embodiment the present invention relates to an expresssion cassette comprising a) a promoter, prefereably a promoter selected from the group consisting of Big35S, PCUbi, Super and USP, in particular USP; and b) the nucleic acid SEQ ID NO. 8363, or a homolog or fragment thereof, or a homolog thereof being depicted in Table I, application no. 1, column 8, in the same line as SEQ ID NO. 8363, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 8363 is depicted, plastidic is mentioned, or a mitochondrial transit petide in case in Table I, application no. 1, in column 6 in the same line as SEQ ID NO. 8363 is depicted, mitochondrial is mentioned; which is operable linke, too.

In another embodiment thereof the expression cassette does not comprise an additional transit peptide in case in Table I, application no. 1, in column 6 in the same line as SEQ ID NO. 8363 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 10708, or a homolog or fragment thereof, or a homolog thereof being depicted in Table I, application no. 1, column 8, in the same line as SEQ ID NO. 10708, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 10708 is depicted, plastidic is mentioned, or a mitochondrial transit petide in case in Table I, application no. 1, in column 6 in the same line as SEQ ID NO. 10708 is depicted, mitochondrial is mentioned; which is operable linke, too.

In another embodiment thereof the expression cassette does not comprise an additional transit peptide in case in Table I, application no. 1, in column 6 in the same line as SEQ ID NO. 10708 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 7333, or a homolog or fragment thereof, or a homolog thereof being depicted in Table I, application no. 1, column 8, in the same line as SEQ ID NO. 7333, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 7333 is depicted, plastidic is mentioned, or a mitochondrial transit petide in case in Table I, application no. 1, in column 6 in the same line as SEQ ID NO. 7333 is depicted, mitochondrial is mentioned; which is operable linke, too.

In another embodiment thereof the expression cassette does not comprise an additional transit peptide in case in Table I, application no. 1, in column 6 in the same line as SEQ ID NO. 7333 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 3654, or a homolog or fragment thereof, or a homolog thereof being depicted in Table I, application no. 1, column 8, in the same line as SEQ ID NO. 3654, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 3654 is depicted, plastidic is mentioned, or a mitochondrial transit petide in case in Table I, application no. 1, in column 6 in the same line as SEQ ID NO. 3654 is depicted, mitochondrial is mentioned; which is operable linke, too.

In another embodiment thereof the expression cassette does not comprise an additional transit peptide in case in Table I, application no. 1, in column 6 in the same line as SEQ ID NO. 3654 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 1061, or a homolog or fragment thereof, or a homolog thereof being depicted in Table I, application no. 1, column 8, in the same line as SEQ ID NO. 1061, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 1061 is depicted, plastidic is mentioned, or a mitochondrial transit petide in case in Table I, application no. 1, in column 6 in the same line as SEQ ID NO. 1061 is depicted, mitochondrial is mentioned; which is operable linke, too.

In another embodiment thereof the expression cassette does not comprise an additional transit peptide in case in Table I, application no. 1, in column 6 in the same line as SEQ ID NO. 1061 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 1298, or a homolog or fragment thereof, or a homolog thereof being depicted in Table I, application no. 1, column 8, in the same line as SEQ ID NO. 1298, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 1298 is depicted, plastidic is mentioned, or a mitochondrial transit petide in case in Table I, application no. 1, in column 6 in the same line as SEQ ID NO. 1298 is depicted, mitochondrial is mentioned; which is operable linke, too.

In another embodiment thereof the expression cassette does not comprise an additional transit peptide in case in Table I, application no. 1, in column 6 in the same line as SEQ ID NO. 1298 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 1623, or a homolog or fragment thereof, or a homolog thereof being depicted in Table I, application no. 1, column 8, in the same line as SEQ ID NO. 1623, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 1623 is depicted, plastidic is mentioned, or a mitochondrial transit petide in case in Table I, application no. 1, in column 6 in the same line as SEQ ID NO. 1623 is depicted, mitochondrial is mentioned; which is operable linke, too.

In another embodiment thereof the expression cassette does not comprise an additional transit peptide in case in Table I, application no. 1, in column 6 in the same line as SEQ ID NO. 1623 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 1696, or a homolog or fragment thereof, or a homolog thereof being depicted in Table I, application no. 1, column 8, in the same line as SEQ ID NO. 1696, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 1696 is depicted, plastidic is mentioned, or a mitochondrial transit petide in case in Table I, application no. 1, in column 6 in the same line as SEQ ID NO. 1696 is depicted, mitochondrial is mentioned; which is operable linke, too.

In another embodiment thereof the expression cassette does not comprise an additional transit peptide in case in Table I, application no. 1, in column 6 in the same line as SEQ ID NO. 1696 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 1815, or a homolog or fragment thereof, or a homolog thereof being depicted in Table I, application no. 1, column 8, in the same line as SEQ ID NO. 1815, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 1815 is depicted, plastidic is mentioned, or a mitochondrial transit petide in case in Table I, application no. 1, in column 6 in the same line as SEQ ID NO. 1815 is depicted, mitochondrial is mentioned; which is operable linke, too.

In another embodiment thereof the expression cassette does not comprise an additional transit peptide in case in Table I, application no. 1, in column 6 in the same line as SEQ ID NO. 1815 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 2367, or a homolog or fragment thereof, or a homolog thereof being depicted in Table I, application no. 1, column 8, in the same line as SEQ ID NO. 2367, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 2367 is depicted, plastidic is mentioned, or a mitochondrial transit petide in case in Table I, application no. 1, in column 6 in the same line as SEQ ID NO. 2367 is depicted, mitochondrial is mentioned; which is operable linke, too.

In another embodiment thereof the expression cassette does not comprise an additional transit peptide in case in Table I, application no. 1, in column 6 in the same line as SEQ ID NO. 2367 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 2573, or a homolog or fragment thereof, or a homolog thereof being depicted in Table I, application no. 1, column 8, in the same line as SEQ ID NO. 2573, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 2573 is depicted, plastidic is mentioned, or a mitochondrial transit petide in case in Table I, application no. 1, in column 6 in the same line as SEQ ID NO. 2573 is depicted, mitochondrial is mentioned; which is operable linke, too.

In another embodiment thereof the expression cassette does not comprise an additional transit peptide in case in Table I, application no. 1, in column 6 in the same line as SEQ ID NO. 2573 is depicted, non-targeted is mentioned.

In an embodiment the present invention relates to an expresssion cassette comprising a) a promoter, prefereably a promoter selected from the group consisting of Big35S, PCUbi, Super and USP, in particular USP; and b) the nucleic acid SEQ ID NO. 10172, or a homolog or fragment thereof, or a homolog thereof being depicted in Table I, application no. 1, column 8, in the same line as SEQ ID NO. 10172, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 10172 is depicted, plastidic is mentioned, or a mitochondrial transit petide in case in Table I, application no. 1, in column 6 in the same line as SEQ ID NO. 10172 is depicted, mitochondrial is mentioned; which is operable linke, too.

In another embodiment thereof the expression cassette does not comprise an additional transit peptide in case in Table I, application no. 1, in column 6 in the same line as SEQ ID NO. 10172 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 3279, or a homolog or fragment thereof, or a homolog thereof being depicted in Table I, application no. 1, column 8, in the same line as SEQ ID NO. 3279, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 3279 is depicted, plastidic is mentioned, or a mitochondrial transit petide in case in Table I, application no. 1, in column 6 in the same line as SEQ ID NO. 3279 is depicted, mitochondrial is mentioned; which is operable linke, too.

In another embodiment thereof the expression cassette does not comprise an additional transit peptide in case in Table I, application no. 1, in column 6 in the same line as SEQ ID NO. 3279 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 385, or a homolog or fragment thereof, or a homolog thereof being depicted in Table I, application no. 1, column 8, in the same line as SEQ ID NO. 385, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 385 is depicted, plastidic is mentioned, or a mitochondrial transit petide in case in Table I, application no. 1, in column 6 in the same line as SEQ ID NO. 385 is depicted, mitochondrial is mentioned; which is operable linke, too.

In another embodiment thereof the expression cassette does not comprise an additional transit peptide in case in Table I, application no. 1, in column 6 in the same line as SEQ ID NO. 385 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 4040, or a homolog or fragment thereof, or a homolog thereof being depicted in Table I, application no. 1, column 8, in the same line as SEQ ID NO. 4040, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 4040 is depicted, plastidic is mentioned, or a mitochondrial transit petide in case in Table I, application no. 1, in column 6 in the same line as SEQ ID NO. 4040 is depicted, mitochondrial is mentioned; which is operable linke, too.

In another embodiment thereof the expression cassette does not comprise an additional transit peptide in case in Table I, application no. 1, in column 6 in the same line as SEQ ID NO. 4040 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 4102, or a homolog or fragment thereof, or a homolog thereof being depicted in Table I, application no. 1, column 8, in the same line as SEQ ID NO. 4102, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 4102 is depicted, plastidic is mentioned, or a mitochondrial transit petide in case in Table I, application no. 1, in column 6 in the same line as SEQ ID NO. 4102 is depicted, mitochondrial is mentioned; which is operable linke, too.

In another embodiment thereof the expression cassette does not comprise an additional transit peptide in case in Table I, application no. 1, in column 6 in the same line as SEQ ID NO. 4102 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 4348, or a homolog or fragment thereof, or a homolog thereof being depicted in Table I, application no. 1, column 8, in the same line as SEQ ID NO. 4348, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 4348 is depicted, plastidic is mentioned, or a mitochondrial transit petide in case in Table I, application no. 1, in column 6 in the same line as SEQ ID NO. 4348 is depicted, mitochondrial is mentioned; which is operable linke, too.

In another embodiment thereof the expression cassette does not comprise an additional transit peptide in case in Table I, application no. 1, in column 6 in the same line as SEQ ID NO. 4348 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 4904, or a homolog or fragment thereof, or a homolog thereof being depicted in Table I, application no. 1, column 8, in the same line as SEQ ID NO. 4904, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 4904 is depicted, plastidic is mentioned, or a mitochondrial transit petide in case in Table I, application no. 1, in column 6 in the same line as SEQ ID NO. 4904 is depicted, mitochondrial is mentioned; which is operable linke, too.

In another embodiment thereof the expression cassette does not comprise an additional transit peptide in case in Table I, application no. 1, in column 6 in the same line as SEQ ID NO. 4904 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 5318, or a homolog or fragment thereof, or a homolog thereof being depicted in Table I, application no. 1, column 8, in the same line as SEQ ID NO. 5318, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 5318 is depicted, plastidic is mentioned, or a mitochondrial transit petide in case in Table I, application no. 1, in column 6 in the same line as SEQ ID NO. 5318 is depicted, mitochondrial is mentioned; which is operable linke, too.

In another embodiment thereof the expression cassette does not comprise an additional transit peptide in case in Table I, application no. 1, in column 6 in the same line as SEQ ID NO. 5318 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 5493, or a homolog or fragment thereof, or a homolog thereof being depicted in Table I, application no. 1, column 8, in the same line as SEQ ID NO. 5493, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 5493 is depicted, plastidic is mentioned, or a mitochondrial transit petide in case in Table I, application no. 1, in column 6 in the same line as SEQ ID NO. 5493 is depicted, mitochondrial is mentioned; which is operable linke, too.

In another embodiment thereof the expression cassette does not comprise an additional transit peptide in case in Table I, application no. 1, in column 6 in the same line as SEQ ID NO. 5493 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 5557, or a homolog or fragment thereof, or a homolog thereof being depicted in Table I, application no. 1, column 8, in the same line as SEQ ID NO. 5557, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 5557 is depicted, plastidic is mentioned, or a mitochondrial transit petide in case in Table I, application no. 1, in column 6 in the same line as SEQ ID NO. 5557 is depicted, mitochondrial is mentioned; which is operable linke, too.

In another embodiment thereof the expression cassette does not comprise an additional transit peptide in case in Table I, application no. 1, in column 6 in the same line as SEQ ID NO. 5557 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 2935, or a homolog or fragment thereof, or a homolog thereof being depicted in Table I, application no. 1, column 8, in the same line as SEQ ID NO. 2935, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 2935 is depicted, plastidic is mentioned, or a mitochondrial transit petide in case in Table I, application no. 1, in column 6 in the same line as SEQ ID NO. 2935 is depicted, mitochondrial is mentioned; which is operable linke, too.

In another embodiment thereof the expression cassette does not comprise an additional transit peptide in case in Table I, application no. 1, in column 6 in the same line as SEQ ID NO. 2935 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 12341, or a homolog or fragment thereof, or a homolog thereof being depicted in Table I, application no. 1, column 8, in the same line as SEQ ID NO. 12341, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 12341 is depicted, plastidic is mentioned, or a mitochondrial transit petide in case in Table I, application no. 1, in column 6 in the same line as SEQ ID NO. 12341 is depicted, mitochondrial is mentioned; which is operable linke, too.

In another embodiment thereof the expression cassette does not comprise an additional transit peptide in case in Table I, application no. 1, in column 6 in the same line as SEQ ID NO. 12341 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 10726, or a homolog or fragment thereof, or a homolog thereof being depicted in Table I, application no. 1, column 8, in the same line as SEQ ID NO. 10726, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 10726 is depicted, plastidic is mentioned, or a mitochondrial transit petide in case in Table I, application no. 1, in column 6 in the same line as SEQ ID NO. 10726 is depicted, mitochondrial is mentioned; which is operable linke, too.

In another embodiment thereof the expression cassette does not comprise an additional transit peptide in case in Table I, application no. 1, in column 6 in the same line as SEQ ID NO. 10726 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 10740, or a homolog or fragment thereof, or a homolog thereof being depicted in Table I, application no. 1, column 8, in the same line as SEQ ID NO. 10740, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 10740 is depicted, plastidic is mentioned, or a mitochondrial transit petide in case in Table I, application no. 1, in column 6 in the same line as SEQ ID NO. 10740 is depicted, mitochondrial is mentioned; which is operable linke, too.

In another embodiment thereof the expression cassette does not comprise an additional transit peptide in case in Table I, application no. 1, in column 6 in the same line as SEQ ID NO. 10740 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 10811, or a homolog or fragment thereof, or a homolog thereof being depicted in Table I, application no. 1, column 8, in the same line as SEQ ID NO. 10811, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 10811 is depicted, plastidic is mentioned, or a mitochondrial transit petide in case in Table I, application no. 1, in column 6 in the same line as SEQ ID NO. 10811 is depicted, mitochondrial is mentioned; which is operable linke, too.

In another embodiment thereof the expression cassette does not comprise an additional transit peptide in case in Table I, application no. 1, in column 6 in the same line as SEQ ID NO. 10811 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 11211, or a homolog or fragment thereof, or a homolog thereof being depicted in Table I, application no. 1, column 8, in the same line as SEQ ID NO. 11211, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 11211 is depicted, plastidic is mentioned, or a mitochondrial transit petide in case in Table I, application no. 1, in column 6 in the same line as SEQ ID NO. 11211 is depicted, mitochondrial is mentioned; which is operable linke, too.

In another embodiment thereof the expression cassette does not comprise an additional transit peptide in case in Table I, application no. 1, in column 6 in the same line as SEQ ID NO. 11211 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 11423, or a homolog or fragment thereof, or a homolog thereof being depicted in Table I, application no. 1, column 8, in the same line as SEQ ID NO. 11423, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 11423 is depicted, plastidic is mentioned, or a mitochondrial transit petide in case in Table I, application no. 1, in column 6 in the same line as SEQ ID NO. 11423 is depicted, mitochondrial is mentioned; which is operable linke, too.

In another embodiment thereof the expression cassette does not comprise an additional transit peptide in case in Table I, application no. 1, in column 6 in the same line as SEQ ID NO. 11423 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 11471, or a homolog or fragment thereof, or a homolog thereof being depicted in Table I, application no. 1, column 8, in the same line as SEQ ID NO. 11471, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 11471 is depicted, plastidic is mentioned, or a mitochondrial transit petide in case in Table I, application no. 1, in column 6 in the same line as SEQ ID NO. 11471 is depicted, mitochondrial is mentioned; which is operable linke, too.

In another embodiment thereof the expression cassette does not comprise an additional transit peptide in case in Table I, application no. 1, in column 6 in the same line as SEQ ID NO. 11471 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 11990, or a homolog or fragment thereof, or a homolog thereof being depicted in Table I, application no. 1, column 8, in the same line as SEQ ID NO. 11990, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 11990 is depicted, plastidic is mentioned, or a mitochondrial transit petide in case in Table I, application no. 1, in column 6 in the same line as SEQ ID NO. 11990 is depicted, mitochondrial is mentioned; which is operable linke, too.

In another embodiment thereof the expression cassette does not comprise an additional transit peptide in case in Table I, application no. 1, in column 6 in the same line as SEQ ID NO. 11990 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 812, or a homolog or fragment thereof, or a homolog thereof being depicted in Table I, application no. 1, column 8, in the same line as SEQ ID NO. 812, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 812 is depicted, plastidic is mentioned, or a mitochondrial transit petide in case in Table I, application no. 1, in column 6 in the same line as SEQ ID NO. 812 is depicted, mitochondrial is mentioned; which is operable linke, too.

In another embodiment thereof the expression cassette does not comprise an additional transit peptide in case in Table I, application no. 1, in column 6 in the same line as SEQ ID NO. 812 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 12140, or a homolog or fragment thereof, or a homolog thereof being depicted in Table I, application no. 1, column 8, in the same line as SEQ ID NO. 12140, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 12140 is depicted, plastidic is mentioned, or a mitochondrial transit petide in case in Table I, application no. 1, in column 6 in the same line as SEQ ID NO. 12140 is depicted, mitochondrial is mentioned; which is operable linke, too.

In another embodiment thereof the expression cassette does not comprise an additional transit peptide in case in Table I, application no. 1, in column 6 in the same line as SEQ ID NO. 12140 is depicted, non-targeted is mentioned.

In an embodiment the present invention relates to an expresssion cassette comprising a) a promoter, prefereably a promoter selected from the group consisting of Big35S, PCUbi, Super and USP, in particular Big35S; and b) the nucleic acid SEQ ID NO. 627, or a homolog or fragment thereof, or a homolog thereof being depicted in Table I, application no. 1, column 8, in the same line as SEQ ID NO. 627, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 627 is depicted, plastidic is mentioned, or a mitochondrial transit petide in case in Table I, application no. 1, in column 6 in the same line as SEQ ID NO. 627 is depicted, mitochondrial is mentioned; which is operable linke, too.

In another embodiment thereof the expression cassette does not comprise an additional transit peptide in case in Table I, application no. 1, in column 6 in the same line as SEQ ID NO. 627 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 12698, or a homolog or fragment thereof, or a homolog thereof being depicted in Table I, application no. 1, column 8, in the same line as SEQ ID NO. 12698, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 12698 is depicted, plastidic is mentioned, or a mitochondrial transit petide in case in Table I, application no. 1, in column 6 in the same line as SEQ ID NO. 12698 is depicted, mitochondrial is mentioned; which is operable linke, too.

In another embodiment thereof the expression cassette does not comprise an additional transit peptide in case in Table I, application no. 1, in column 6 in the same line as SEQ ID NO. 12698 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 12974, or a homolog or fragment thereof, or a homolog thereof being depicted in Table I, application no. 1, column 8, in the same line as SEQ ID NO. 12974, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 12974 is depicted, plastidic is mentioned, or a mitochondrial transit petide in case in Table I, application no. 1, in column 6 in the same line as SEQ ID NO. 12974 is depicted, mitochondrial is mentioned; which is operable linke, too.

In another embodiment thereof the expression cassette does not comprise an additional transit peptide in case in Table I, application no. 1, in column 6 in the same line as SEQ ID NO. 12974 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 13376, or a homolog or fragment thereof, or a homolog thereof being depicted in Table I, application no. 1, column 8, in the same line as SEQ ID NO. 13376, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 13376 is depicted, plastidic is mentioned, or a mitochondrial transit petide in case in Table I, application no. 1, in column 6 in the same line as SEQ ID NO. 13376 is depicted, mitochondrial is mentioned; which is operable linke, too.

In another embodiment thereof the expression cassette does not comprise an additional transit peptide in case in Table I, application no. 1, in column 6 in the same line as SEQ ID NO. 13376 is depicted, non-targeted is mentioned.

In an embodiment the present invention relates to an expresssion cassette comprising a) a promoter, prefereably a promoter selected from the group consisting of Big35S, PCUbi, Super and USP, in particular Big35S; and b) the nucleic acid SEQ ID NO. 14171, or a homolog or fragment thereof, or a homolog thereof being depicted in Table I, application no. 1, column 8, in the same line as SEQ ID NO. 14171, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 14171 is depicted, plastidic is mentioned, or a mitochondrial transit petide in case in Table I, application no. 1, in column 6 in the same line as SEQ ID NO. 14171 is depicted, mitochondrial is mentioned; which is operable linke, too.

In another embodiment thereof the expression cassette does not comprise an additional transit peptide in case in Table I, application no. 1, in column 6 in the same line as SEQ ID NO. 14171 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 14275, or a homolog or fragment thereof, or a homolog thereof being depicted in Table I, application no. 1, column 8, in the same line as SEQ ID NO. 14275, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 14275 is depicted, plastidic is mentioned, or a mitochondrial transit petide in case in Table I, application no. 1, in column 6 in the same line as SEQ ID NO. 14275 is depicted, mitochondrial is mentioned; which is operable linke, too.

In another embodiment thereof the expression cassette does not comprise an additional transit peptide in case in Table I, application no. 1, in column 6 in the same line as SEQ ID NO. 14275 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 69, or a homolog or fragment thereof, or a homolog thereof being depicted in Table I, application no. 1, column 8, in the same line as SEQ ID NO. 69, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 69 is depicted, plastidic is mentioned, or a mitochondrial transit petide in case in Table I, application no. 1, in column 6 in the same line as SEQ ID NO. 69 is depicted, mitochondrial is mentioned; which is operable linke, too.

In another embodiment thereof the expression cassette does not comprise an additional transit peptide in case in Table I, application no. 1, in column 6 in the same line as SEQ ID NO. 69 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 352, or a homolog or fragment thereof, or a homolog thereof being depicted in Table I, application no. 1, column 8, in the same line as SEQ ID NO. 352, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 352 is depicted, plastidic is mentioned, or a mitochondrial transit petide in case in Table I, application no. 1, in column 6 in the same line as SEQ ID NO. 352 is depicted, mitochondrial is mentioned; which is operable linke, too.

In another embodiment thereof the expression cassette does not comprise an additional transit peptide in case in Table I, application no. 1, in column 6 in the same line as SEQ ID NO. 352 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 6040, or a homolog or fragment thereof, or a homolog thereof being depicted in Table I, application no. 1, column 8, in the same line as SEQ ID NO. 6040, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 6040 is depicted, plastidic is mentioned, or a mitochondrial transit petide in case in Table I, application no. 1, in column 6 in the same line as SEQ ID NO. 6040 is depicted, mitochondrial is mentioned; which is operable linke, too.

In another embodiment thereof the expression cassette does not comprise an additional transit peptide in case in Table I, application no. 1, in column 6 in the same line as SEQ ID NO. 6040 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 12070, or a homolog or fragment thereof, or a homolog thereof being depicted in Table I, application no. 1, column 8, in the same line as SEQ ID NO. 12070, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 1, in column 6 in the same line as SEQ ID NO. 12070 is depicted, plastidic is mentioned, or a mitochondrial transit petide in case in Table I, application no. 1, in column 6 in the same line as SEQ ID NO. 12070 is depicted, mitochondrial is mentioned; which is operable linke, too.

In another embodiment thereof the expression cassette does not comprise an additional transit peptide in case in Table I, application no. 1, in column 6 in the same line as SEQ ID NO. 12070 is depicted, non-targeted is mentioned.

Accordingly, in one embodiment, the process according to the invention comprises the following steps:
(a) introducing of a nucleic acid construct comprising the nucleic acid molecule of the invention (and if desired, in combination with the nucleic acid encoding a respective transit peptide) or used in the process of the invention or encoding the polypeptide of the present invention or used in the process of the invention; or
(b) introducing of a nucleic acid molecule, including regulatory sequences and/or factors(and if desired, in combination with the nucleic acid encoding a respective transit peptide), which expression increases the expression of the nucleic acid molecule of the invention or used in the process of the invention or encoding the polypeptide of the present invention or used in the process of the invention;
in a cell, or a non-human organism or a part thereof, preferably in a plant, plant cell or a microorganism, preferably in the organelles such as the plastids or mitochondria thereof, or preferably non-targeted, or
(c) introducing an expression cassette as mentioned above, and
(d) expressing of the gene product encoded by the nucleic acid construct or the nucleic acid molecule (and if desired, in combination with the nucleic acid encoding a respective transit peptide) or expresion cassette mentioned under (a), (b) or (c) in the cell or the non-human organism or part thereof.

For expression in a host organism, for example a plant, the expression cassette is advantageously inserted into a vector such as by way of example a plasmid, a phage or other DNA which allows optimal expression of the genes in the host organism. Examples of suitable plasmids are: in E. coli pLG338, pACYC184, pBR series such as e.g. pBR322, pUC series such as pUC18 or pUC19, M113mp series, pKC30, pRep4, pHS1, pHS2, pPLc236, pMBL24, pLG200, pUR290, pIN-III113-B1, λgt11 or pBdCI; in Streptomyces pIJ101, pIJ364, pIJ702 or pIJ361; in Bacillus pUB110, pC194 or pBD214; in Corynebacterium pSA77 or pAJ667; in fungi pALS1, pIL2 or pBB116; other advantageous fungal vectors are described by Romanos M. A. et al., Yeast 8, 423 (1992) and by van den Hondel C. A. M. J. J. et al. [(1991) “Heterologous gene expression in filamentous fungi”] as well as in “More Gene Manipulations” in “Fungi” in Bennet J. W. & Lasure L. L., eds., pp. 396-428, Academic Press, San Diego, and in “Gene transfer systems and vector development for filamentous fungi” [van den Hondel C. A. M. J. J. & Punt P. J. (1991) in: Applied Molecular Genetics of Fungi, Peberdy J. F. et al., eds., pp. 1-28, Cambridge University Press: Cambridge]. Examples of advantageous yeast vectors are 2pM, pAG-1, YEp6, YEp13 or pEMBLYe23. Examples of algal or plant plasmids are pLGV23, pGHIac+, pBIN19, pAK2004, pVKH or pDH51 (see Schmidt R., Willmitzer L., Plant Cell Rep. 7, 583 (1988)). The vectors identified above or derivatives of the vectors identified above are a small selection of the possible plasmids. Further plasmids are well known to those skilled in the art and may be found, for example, in “Cloning Vectors” (Eds. P. H. Pouwels et al., Elsevier, Amsterdam-New York-Oxford, 1985, ISBN 0 444 904018). Suitable plant vectors are described inter alia in “Methods in Plant Molecular Biology and Biotechnology” (CRC Press, Ch. 6/7, pp. 71-119). Advantageous vectors are known as shuttle vectors or binary vectors which replicate in E. coli and Agrobacterium. Examples for binary vectors are pBIN19, pBI101, pBinAR, pGPTV, pCAMBIA, pBIB-HYG, pBecks, pGreen or pPZP (Hajukiewicz P. et al., Plant Mol. Biol. 25, 989 (1994), and Hellens et al, Trends in Plant Science 5, 446 (2000)).

In a further embodiment of the vector the expression cassette according to the invention may also advantageously be introduced into the organisms in the form of a linear DNA and be integrated into the genome of the host organism by way of heterologous or homologous recombination. This linear DNA may be composed of a linearized plasmid or only of the expression cassette as vector or the respective nucleic acid sequences (if desired, in combination with the nucleic acid encoding a respective transit peptide) according to the invention.

In a further advantageous embodiment the nucleic acid sequence (if desired, in combination with the nucleic acid encoding a respective transit peptide) according to the invention can also be introduced into an organism on its own.

If in addition to the nucleic acid sequence according to the invention (and if desired, in combination with the nucleic acid encoding a respective transit peptide) further genes are to be introduced into the organism, all genes altogether with a marker in a single vector or each single gene with a marker in a vector can be introduced into the organism, whereby the different vectors can be introduced simultaneously or successively.

The vector advantageously contains at least one copy of the nucleic acid sequences according to the invention (and if desired, in combination with the nucleic acid encoding a respective transit peptide) or of the expression cassette (=gene construct) according to the invention.

The invention further provides a vector, comprising a nucleic acid molecule comprising 1) a nucleic acid molecule selected from the group consisting of:
(a) a nucleic acid molecule encoding a polypeptide shown in Table II, column 5 or 8, application no. 1, preferably shown in Table IIA, application no. 1, in column 5, or in Table II A, application no. 1, column 8, or in Table II B, application no. 1, column 8, or a homolog or a fragment thereof;
(b) a nucleic acid molecule shown in Table I, application no. 1, column 5 or 8, preferably shown in Table I A, application no. 1, in column 5, or in Table I A, application no. 1, column 8, or in Table I B, application no. 1, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 1, preferably shown in Table II A, application no. 1, in column 5, or in Table II A, application no. 1, column 8, or in Table II B, application no. ,1, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 1, preferably shown in Table I A, application no. 1, in column 5, or in Table I A, application no. 1, column 8, or in Table I B, application no. 1,column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 1;
(i) a nucleic acid molecule encoding a polypeptide having the activity represented by a protein as depicted in column 5 of Table II, application no. 1;
(j) a nucleic acid molecule comprising a polynucleotide which is obtained by amplifying a cDNA library or a genomic library using the primers in Table III, application no. 1, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
   or a nucleic acid molecule comprising a sequence which is complementary thereto.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 1.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 1, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 1.

In another preferred embodiment thereof said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 1, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 1, in the same line as of the respective nucleic acid molecule according to 1) “plastidic” is depicted, or encoding a mitochondrial transit peptide, especially in case in column 6 of Table I, application no. 1, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 1, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 1, in the same line as of the respective nucleic acid molecule according to 1) “plastidic” is depicted, or encoding a mitochondrial transit peptide, especially in case in column 6 of Table I, application no. 1, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP, which are operable linked; a nucleic acid molecule encoding a transit peptide, especially in case in column 6 of Table I, application no. 1, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 1, in the same line as of the respective nucleic acid molecule according to 1) “plastidic” is depicted, or encoding a mitochondrial transit peptide, especially in case in column 6 of Table I, application no. 1, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 1 (in the same line).

In another embodiment thereof said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP, which are operatively linked; a nucleic acid molecule encoding a transit peptide, especially in case in column 6 of Table I, application no. 1, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 1.

In another embodiment thereof said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 1, in the same line as of the respective nucleic acid molecule according to 1) “plastidic” is depicted, or encoding a mitochondrial transit peptide, especially in case in column 6 of Table I, application no. 1, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP, which are operatively linked; a nucleic acid molecule encoding a transit peptide, especially in case in column 6 of Table I, application no. 1, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 1, in the same line as of the respective nucleic acid molecule according to 1) “plastidic” is depicted, or encoding a mitochondrial transit peptide, especially in case in column 6 of Table I, application no. 1, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 1 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP, which are operatively linked; a nucleic acid molecule encoding a transit peptide, especially in case in column 6 of Table I, application no. 1, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 1 (in the same line) and confers a generation or an increase of the respective fine chemical in a non-human organism or a part thereof as compared to a corresponding wild-type non-human organism or part thereof.

In a prefered embodiment of said vector said nucleic acid molecule according to 1 (a), (b), (c), (d), (e), (f), (g), (h), (i), (j) or (k), said promoter and optionally said nucleic acid molecule encoding a transit peptide are encompassed in an expression cassette. Accordingly in an embodiment the invention provides a vector comprising an expression casssette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

The invention further provides a vector comprising a nucleic acid molecule comprising 1) a nucleic acid molecule selected from the group consisting of:
(a) a nucleic acid molecule encoding a polypeptide shown in Table II, column 5 or 8, application no. 1, preferably shown in Table II A, application no. 1, in column 5, or in Table II A, application no. 1, column 8, or in Table II B, application no. 1, column 8, or a homolog or a fragment thereof;
(b) a nucleic acid molecule shown in Table I, application no. 1, column 5 or 8, preferably shown in Table I A, application no. 1, in column 5, or in Table I A, application no. 1, column 8, or in Table I B, application no. 1, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 1, preferably shown in Table II A, application no. 1, in column 5, or in Table II A, application no. 1, column 8, or in Table II B, application no. 1, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 1, preferably shown in Table I A, application no. 1, in column 5, or in Table I A, application no. 1, column 8, or in Table I B, application no. 1, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);

(h) a nucleic acid molecule encoding a polypeptide comprising the consensus sequence or one or more polypeptide motifs as shown in column 8 of Table IV, application no. 1;
(i) a nucleic acid molecule encoding a polypeptide having the activity represented by a protein as depicted in column 5 of Table II, application no. 1;
(j) a nucleic acid molecule comprising a polynucleotide which is obtained by amplifying a cDNA library or a genomic library using the primers in Table III, application no. 1, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
   or a nucleic acid molecule comprising a sequence which is complementary thereto;
   whereby, preferably, the nucleic acid molecule according to 1 (a), (b), (c), (d), (e), (f), (g), (h), (i), (j) or (k) distinguishes over the sequence depicted in Table I A and/or I B, application no. 1, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 1, column 5 or 8, or the coding region thereof, respectively. In another embodiment, the nucleic acid molecule of the present invention, preferably the coding region thereof, is 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 1, column 5 or 8, or the coding region thereof, respectively. In a further embodiment the nucleic acid molecule does not encode the polypeptide sequence shown in Table II A and/or II B, application no. 1, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 1, column 5 or 8 and does not encode a protein of the sequence shown in Table II A and/or II B, application no. 1, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 1, column 5 and 8, or the coding region thereof. In a further embodiment, the protein of the present invention is at least 30%,40%, 50%, 60%, 70%, 80%, 90%, 95% identical to protein sequence depicted in

Table II A and/or II B, application no. 1, column 5 or 8 but less than 100%, preferably less than 99.999%, 99.99% or 99.9%, more preferably less than 99%, 985, 97%, 96% or 95% identical to the sequence shown in Table II A and/or II B, application no. 1, column 5 or 8.

(All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 1.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 1, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 1.

In another preferred embodiment thereof said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 1, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 1, in the same line as of the respective nucleic acid molecule according to 1) “plastidic” is depicted, or encoding a mitochondrial transit peptide, especially in case in column 6 of Table I, application no. 1, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 1, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 1, in the same line as of the respective nucleic acid molecule according to 1) “plastidic” is depicted, or encoding a mitochondrial transit peptide, especially in case in column 6 of Table I, application no. 1, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP, which are operatively linked; a nucleic acid molecule encoding a transit peptide, especially in case in column 6 of Table I, application no. 1, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 1, in the same line as of the respective nucleic acid molecule according to 1) “plastidic” is depicted, or encoding a mitochondrial transit peptide, especially in case in column 6 of Table I, application no. 1, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 1.

In another embodiment thereof said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP, which are operatively linked; a nucleic acid molecule encoding a transit peptide, especially in case in column 6 of Table I, application no. 1, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 1.

In another embodiment thereof said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 1, in the same line as of the respective nucleic acid molecule according to 1) “plastidic” is depicted, or encoding a mitochondrial transit peptide, especially in case in column 6 of Table I, application no. 1, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP, which are operatively linked; a nucleic acid molecule encoding a transit peptide, especially in case in column 6 of Table I, application no. 1, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 1, in the same line as of the respective nucleic acid molecule according to 1) “plastidic” is depicted, or encoding a mitochondrial transit peptide, especially in case in column 6 of Table I, application no. 1, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 1 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP, which are operatively linked; a nucleic acid molecule encoding a transit peptide, especially in case in column 6 of Table I, application no. 1, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 1 and confers a generation or an increase of the respective fine chemical in a non-human organism or a part thereof as compared to a corresponding wild-type non-human organism or part thereof.

In a prefered embodiment of said vector said nucleic acid molecule according to 1 (a), (b), (c), (d), (e), (f), (g), (h), (i), (j) or (k), said promoter and optionally said nucleic acid molecule encoding a transit peptide are encompassed in an expression cassette. Accordingly in an embodiment the invention provides a vector comprising an expression casssette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

The invention further provides a vector, especially an expression vector, comprising a FCRP encoding nucleic acid as described above, wherein expression of the vector or FCRP encoding nucleic acid, respectively in a host cell results in the production of a the respective fine chemical, preferably in an increased production of the respective fine chemical, as compared to a wild type of the host cell.

As used herein, the term “vector” refers to a nucleic acid molecule capable of transporting another nucleic acid which has been linked thereto. One type of vector is a “plasmid”, which refers to a circular double stranded DNA loop into which additional DNA segments can be ligated. Another type of vector is a viral vector, wherein additional DNA segments can be ligated into the viral genome. Further types of vectors can be linearized nucleic acid sequences, such as transposons, which are pieces of DNA which can copy and insert themselves. There have been two types of transposons found: simple transposons, known as Insertion Sequences and composite transposons, which can have several genes as well as the genes that are required for transposition. Certain vectors are capable of autonomous replication in a host cell into which they are introduced (e.g. bacterial vectors having a bacterial origin of replication and episomal mammalian vectors). Other vectors (e.g. non-episomal mammalian vectors) are integrated into the genome of a host cell or an organelle upon introduction into the host cell, and thereby are replicated along with the host or organelle genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include other forms of expression vectors, such as viral vectors (e.g., replication defective retroviruses, adenoviruses, and adeno-associated viruses), which exert similar functions.

The term vector furthermore encompasses other vectors known to those skilled in the art such as by way of example phages, viruses such as SV40, CMV, baculovirus, adenovirus, transposons, IS elements, phasmids, phagemids, cosmids, linear or circular DNA.

These vectors can be replicated autonomously in the host organism or be chromosomally replicated, chromosomal replication being preferred.

The recombinant expression vectors of the invention comprise a nucleic acid molecule of the invention in a form suitable for expression of the nucleic acid molecule in a host cell, which means that the recombinant expression vectors include one or more regulatory sequences, selected on the basis of the host cells to be used for expression, and, if desired a targeting sequence, which are operatively linked to the nucleic acid sequence to be expressed. As used herein with respect to a recombinant expression vector, “operatively linked” is intended to mean that the nucleotide sequence of interest is linked to the regulatory sequence(s)/targeting sequence in a manner which allows for expression of the nucleotide sequence in such way that the predicted function assigned to the nucleic sequence is fulfilled (e.g. in an in vitro transcription/translation system or in a host cell when the vector is introduced into the host cell). The term “regulatory sequence” is intended to include promoters, enhancers, and other expression control elements (e.g. polyadenylation signals). Such regulatory sequences are described, for example, in Goeddel “Gene Expression Technology: Methods in Enzymology 185”, Academic Press, San Diego, Calif. (1990), and Gruber and Crosby, in: Methods in Plant Molecular Biology and Biotechnology, eds. Glick and Thompson, Chapter 7, 89-108, CRC Press; Boca Raton, Fla., including the references therein. Regulatory sequences include those that direct constitutive expression of a nucleotide sequence in many types of host cells and those that direct expression of the nucleotide sequence only in certain host cells or under certain conditions. It will be appreciated by those skilled in the art that the design of the expression vector can depend on such factors as the choice of the host cell to be transformed, the level of expression of polypeptide desired, etc. The term “regulatory sequence” is to be considered as being encompassed by the the term “regulatory signal”. The expression vectors of the invention can be introduced into host cells to thereby produce polypeptides or peptides, including fusion polypeptides or peptides, encoded by nucleic acid molecules as described herein (e.g., fusion polypeptides, FCRPs etc.).

In another embodiment the recombinant expression vectors of the invention can be designed specifically for expression of the polypeptides of the invention in plant cells. For example, FCRP genes can be expressed in plant cells (see R. Schmidt, L. Willmitzer, Plant Cell Rep. 7, 583 (1988); Plant Molecular Biology and Biotechnology, C Press, Boca Raton, Fla., Chapter 6/7, p. 71-119 (1993); F. F. White, B. Jenes et al., Techniques for Gene Transfer, in: Transgenic Plants, Vol. 1, Engineering and Utilization, eds. Kung and Wu R., 128-43, Academic Press: 1993; Potrykus, Annu. Rev. Plant Physiol. Plant Molec. Biol. 42, 205 (1991) and references cited therein). Suitable host cells are discussed further in Goeddel, “Gene Expression Technology: Methods in Enzymology 185”, Academic Press: San Diego, Calif. (1990). By way of example the plant expression cassette can be installed in the pRT transformation vector ((a) Toepfer et al., Methods Enzymol. 217, 66 (1993), (b) Toepfer et al., Nucl. Acids. Res. 15, 5890 (1987)). Alternatively, the recombinant expression vector can be transcribed and translated in vitro, for example using T7 promoter regulatory sequences and T7 polymerase.

Expression vectors employed in prokaryotes frequently make use of inducible systems with and without fusion proteins or fusion oligopeptides, wherein these fusions can ensue in both N-terminal and C-terminal manner or in other useful domains of a protein. Such fusion vectors usually have the following purposes: 1) to increase the RNA expression rate; 2) to increase the achievable protein synthesis rate; 3) to increase the solubility of the protein; 4) or to simplify purification by means of a binding sequence usable for affinity chromatography. Proteolytic cleavage points are also frequently introduced via fusion proteins, which allow cleavage of a portion of the fusion protein and purification. Such recognition sequences for proteases are recognized, e.g. factor Xa, thrombin and enterokinase.

Typical advantageous fusion and expression vectors are pGEX (Pharmacia Biotech Inc; D. B. Smith and K.S. Johnson, Gene 67, 31 (1988)), pMAL (New England Biolabs, Beverly, Mass.) and pRIT5 (Pharmacia, Piscataway, N.J.) which contains glutathione S-transferase (GST), maltose binding protein or protein A.

In an embodiment of the present invention, the coding sequence for the polypeptide of the invention is cloned into a pGEX expression vector to create a vector encoding a fusion polypeptide comprising, from the N-terminus to the C-terminus, GST-thrombin cleavage site-X polypeptide. The fusion polypeptide can be purified by affinity chromatography using glutathione-agarose resin. Recombinant FCRP unfused to GST can be recovered by cleavage of the fusion polypeptide with thrombin. Other examples of E. coli expression vectors are pTrc (Amann et al., Gene 69, 301 (1988)) and pET vectors (Studier et al., “Gene Expression Technology: Methods in Enzymology 185”, Academic Press, San Diego, Calif. (1990) 60-89; Stratagene, Amsterdam, The Netherlands).

Target gene expression from the pTrc vector relies on host RNA polymerase transcription from a hybrid trp-lac fusion promoter. Target gene expression from the pET 11d vector relies on transcription from a T7 gn10-lac fusion promoter mediated by a co-expressed viral RNA polymerase (T7 gn1). This viral polymerase is supplied by host strains BL21(DE3) or HMS174(DE3) from a resident I prophage harboring a T7 gn1 gene under the transcriptional control of the lacUV 5 promoter.

In a further embodiment of the present invention, the FCRPs are expressed in plants and plant cells such as unicellular plant cells (e.g. algae) (see Falciatore et al., Marine Biotechnology 1 (3), 239 (1999) and references therein) and plant cells from higher plants (e.g., the spermatophytes, such as crop plants), for example to regenerate plants from the plant cells. A nucleic acid molecule coding for FCRP as depicted in Table II, column 5 or 8, or a homolog or a fragment thereof, (and, if desired, in combination with the nucleic acid encoding a respective transit peptide) may be “introduced” into a plant cell by any means, including transfection, transformation or transduction, electroporation, particle bombardment, agroinfection, and the like. One transformation method known to those of skill in the art is the dipping of a flowering plant into an Agrobacteria solution, wherein the Agrobacteria contains the nucleic acid of the invention, followed by breeding of the transformed gametes.

Other suitable methods for transforming or transfecting host cells including plant cells can be found in Sambrook et al., “Molecular Cloning: A Laboratory Manual”. 2nd ed., Cold Spring Harbor Laboratory, Cold Spring Harbor Laboratory Press, Cold Spring Harbor, N.Y., 1989, and other laboratory manuals such as “Methods in Molecular Biology”, 1995, Vol. 44, Agrobacterium protocols, ed: Gartland and Davey, Humana Press, Totowa, N. J. As generated or increased production of the fine chemical is generally wished to be inherited into a wide variety of monocotyledonous or dicotyledonous plants, preferably plants like maize, wheat, rye, oat, triticale, rice, barley, sugar cane, soybean, peanut, cotton, rapeseed and canola, manihot, pepper, sunflower and tagetes, sugar beet, solanaceous plants like potato, tobacco, eggplant, and tomato, Vicia species, pea, alfalfa, bushy plants (coffee, cacao, tea), Salix species, trees (oil palm, coconut), perennial grasses, and forage crops, these crop plants are also preferred target plants for a genetic engineering as one further embodiment of the present invention. Forage crops include, but are not limited to Wheatgrass, Canarygrass, Bromegrass, Wildrye Grass, Bluegrass, Orchardgrass, Alfalfa, Salfoin, Birdsfoot Trefoil, Alsike Clover, Red Clover and Sweet Clover.

In one embodiment of the present invention, transfection of a nucleic acid molecule coding for FCRP e.g. as depicted in Table II column 5 or 8, or a homolog or a fragment thereof,(and, if desired, in combination with the nucleic acid encoding a respective transit peptide) into a plant is achieved by Agrobacterium mediated gene transfer. Agrobacterium mediated plant transformation can be performed using for example the GV3101(pMP90) (Koncz and Schell, Mol. Gen. Genet. 204, 383 (1986)) or LBA4404 (Clontech) Agrobacterium tumefaciens strain. Transformation can be performed by standard transformation and regeneration techniques (Deblaere et al., Nucl. Acids Res. 13, 4777 (1994), Gelvin, B. Stanton and Robert A.

Schilperoort, Plant Molecular Biology Manual, 2nd ed.—Dordrecht: Kluwer Academic Publ., 1995.—in Sect., Ringbuc Zentrale Signatur: BT11-P ISBN 0-7923-2731-4; Bernard R. Glick., John E. Thompson, “Methods in Plant Molecular Biology and Biotechnology”, Boca Raton: CRC Press, 1993 p. 360, ISBN 0-8493-5164-2). For example, rapeseed can be transformed via cotyledon or hypocotyl transformation (Moloney et al., Plant Cell Report 8, 238 (1989); De Block et al., Plant Physiol. 91, 694 (1989)). Use of antibiotics for Agrobacterium and plant selection depends on the binary vector and the Agrobacterium strain used for transformation. Rapeseed selection is normally performed using kanamycin as selectable plant marker. Agrobacterium mediated gene transfer to flax can be performed using, for example, a technique described by Mlynarova et al., Plant Cell Report 13, 282 (1994). Additionally, transformation of soybean can be performed using for example a technique described in European Patent No. 424 047, U.S. Pat. No. 5,322,783, European Patent No. 397 687, U.S. Pat. No. 5,376,543 or U.S. Pat. No. 5,169,770. Transformation of maize can be achieved by particle bombardment, polyethylene glycol mediated DNA uptake or via the silicon carbide fiber technique. (See, for example, Freeling and Walbot “The maize handbook” Springer Verlag: New York (1993) ISBN 3-540-97826-7). A specific example of maize transformation is found in U.S. Pat. No. 5,990,387, and a specific example of wheat transformation can be found in PCT Application No. WO 93/07256. Examples for rice transformation can be found e.g. in EP 1 728 418 or EP 897 013.

According to the present invention, the introduced nucleic acid molecule coding for FCRP e.g. as depicted in Table II, column 5 or 8, or a homolog or a fragment thereof, may be maintained in the plant cell stably if it is incorporated into a non-chromosomal autonomous replicon or integrated into the plant chromosomes or organelle genome. Alternatively, the introduced FCRP may be present on an extra-chromosomal non-replicating vector and be transiently expressed or transiently active.

Whether present in an extra-chromosomal non-replicating vector or a vector that is integrated into a chromosome, the nucleic acid molecule coding for FCRP as depicted in Table II, column 5 or 8, a homolog or a fragment thereof, preferably resides in a non-human organism expression cassette, preferably a microorganism expression cassette or a plant expression cassette. A plant expression cassette preferably contains regulatory sequences capable of driving gene expression in plant cells that are operatively linked so that each sequence can fulfill its function, for example, termination of transcription by polyadenylation signals. Preferred polyadenylation signals are those originating from Agrobacterium tumefaciens t-DNA such as the gene 3 known as octopine synthase of the Ti-plasmid pTiACH5 (Gielen et al., EMBO J. 3, 835 (1984)) or functional equivalents thereof but also all other terminators functionally active in plants are suitable. As plant gene expression is very often not limited on transcriptional levels, a plant expression cassette preferably contains other operatively linked sequences like translational enhancers such as the overdrive-sequence containing the 5″-untranslated leader sequence from tobacco mosaic virus enhancing the polypeptide per RNA ratio (Gallie et al., Nucl. Acids Research 15, 8693 (1987)). Examples of plant expression vectors include those detailed in: D. Becker et al., Plant Mol. Biol. 20, 1195 (1992); and M. W. Bevan, Nucl. Acid. Res. 12, 8711 (1984); and “Vectors for Gene Transfer in Higher Plants” in: Transgenic Plants, Vol. 1, Engineering and Utilization, eds. Kung and Wu R., Academic Press, 1993, S. 15-38.

“Transformation” is defined herein as a process for introducing DNA into a non-human organism, preferably a microrganism or a plant or a part thereof, like a plant cell or plant tissue. In an embodiment the transformation is performed with heterologous DNA. In another embodiment the transformation is performed with “additional” homologous DNA. Transformation may occur under natural or artificial conditions using various methods well known in the art. Transformation may rely on any known method for the insertion of (foreign, additional) nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome. They also include cells which transiently express the inserted DNA or RNA for limited periods of time. Transformed plant cells, plant tissue, or plants are understood to encompass not only the end product of a transformation process, but also transgenic progeny thereof.

The terms “transformed,” “transgenic,” and “recombinant” refer to a host organism such as a microorganism, e.g. a bacterium, or a plant into which a nucleic acid molecule has been introduced. In an embodiment the introduced nucleic acid molecule is heterogenous. In another embodiment the introduced DNA is homologous. The nucleic acid molecule can be stably integrated into the genome of the host or the nucleic acid molecule can also be present as an extra-chromosomal molecule. Such an extra-chromosomal molecule can be autoreplicating. A “non-transformed”, “non-transgenic” or “non-recombinant” host refers to a wild-type organism, e.g. a bacterium or plant, which does not contain the heterologous nucleic acid molecule or the additional homologous nucleic acid molecule.

In an embodiment a “transgenic plant”, as used herein, refers to a plant which contains a nucleotide sequence inserted into either its nuclear genome or organelle genome. In an embodiment the introduced nucleic acid molecule is heterogenous. In another embodiment the introduced DNA is homologous. In each case it encompasses further the offspring generations i.e. the Ti-, T2- and consecutively generations or BC1-, BC2- and consecutively generation as well as crossbreeds thereof with non-transgenic or other transgenic plants, as long as it contains said nucleic acid sequence.

The host organism (=transgenic organism) advantageously contains at least one copy of the nucleic acid in addition according to the invention and/or of the nucleic acid construct in addition according to the invention.

In principle all non-human organism can be used as host organism. In an embodiment the transgenic non-human organism or cell is an prokaryotic organism. In an embodiment the transgenic non-human organism or cell is an eukaryotic organism, like an algae, a non-human animal or a plant, in particular an algae or a plant. Preferred transgenic plants are monocotyledonous or dicotyledonous plants, preferably, for example, selected from the families Aceraceae, Anacardiaceae, Apiaceae, Asteraceae, Brassicaceae, Cactaceae, Cucurbitaceae, Euphorbiaceae, Fabaceae, Malvaceae, Nymphaeaceae, Papaveraceae, Rosaceae, Salicaceae, Solanaceae, Arecaceae, Bromeliaceae, Cyperaceae, lridaceae, Liliaceae, Orchidaceae, Gentianaceae, Labiaceae, Magnoliaceae, Ranunculaceae, Carifolaceae, Rubiaceae, Scrophulariaceae, Caryophyllaceae, Ericaceae, Polygonaceae, Violaceae, Juncaceae or Poaceae and preferably from a plant selected from the group of the families Apiaceae, Asteraceae, Brassicaceae, Cucurbitaceae, Fabaceae, Papaveraceae, Rosaceae, Solanaceae, Liliaceae or Poaceae. Preferred are crop plants such as plants advantageously selected from the group of the genus peanut, oilseed rape, canola, sunflower, safflower, olive, sesame, hazelnut, almond, avocado, bay, pumpkin/squash, linseed, soya, pistachio, borage, maize, wheat, rye, oats, sorghum and millet, triticale, rice, barley, sugarcane, cotton, cassava, potato, sugarbeet, egg plant, alfalfa, and perennial grasses and forage plants, oil palm, vegetables (brassicas, root vegetables, tuber vegetables, pod vegetables, fruiting vegetables, onion vegetables, leafy vegetables and stem vegetables), buckwheat, Jerusalem artichoke, broad bean, vetches, lentil, dwarf bean, lupin, clover and lucerne for mentioning only some of them.

In one embodiment of the invention transgenic plants are selected from the group comprising monocotyledonous or dicotyledonous plants, preferably cereals, soybean, rapeseed (including oil seed rape, especially canola and winter oil seed rape), cotton, sugarcane and potato, especially corn, soy, rapeseed (including oil seed rape, especially canola and winter oil seed rape), cotton, wheat and rice.

In one embodiment, the transgenic plant is a crop plant, in particular a transgenic plant belonging to e.g. Anacardium occidentale, Calendula officinalis, Carthamus tinctorius, Cichorium intybus, Cynara scolymus, Helianthus annus, Tagetes lucida, Tagetes erecta, Tagetes tenuifolia; Daucus carota; Corylus avellana, Corylus colurna, Borago officinalis; Brassica napus, Brassica rapa ssp., Sinapis arvensis Brassica juncea, Brassica juncea var. juncea, Brassica juncea var. crispifolia, Brassica juncea var. foliosa, Brassica nigra, Brassica sinapioides, Melanosinapis communis, Brassica oleracea, Arabidopsis thaliana, Anana comosus, Ananas ananas, Bromelia comosa, Carica papaya, Cannabis sative, Ipomoea batatus, Ipomoea pandurata, Convolvulus batatas, Convolvulus tiliaceus, Ipomoea fastigiata, Ipomoea tiliacea, Ipomoea triloba, Convolvulus panduratus, Beta vulgaris, Beta vulgaris var. altissima, Beta vulgaris var. vulgaris, Beta maritima, Beta vulgaris var. perennis, Beta vulgaris var. conditiva, Beta vulgaris var. esculenta, Cucurbita maxima, Cucurbita mixta, Cucurbita pepo, Cucurbita moschata, Olea europaea, Manihot utilissima, Janipha manihot, Jatropha manihot., Manihot aipil, Manihot dulcis, Manihot manihot, Manihot melanobasis, Manihot esculenta, Ricinus communis, Pisum sativum, Pisum arvense, Pisum humile, Medicago sativa, Medicago falcata, Medicago varia, Glycine max Dolichos soja, Glycine gracilis, Glycine hispida, Phaseolus max, Soja hispida, Glycine max, Cocos nucifera, Pelargonium grossularioides, Oleum cocoas, Laurus nobilis, Persea americana, Arachis hypogaea, Linum usitatissimum, Linum humile, Linum austriacum, Linum bienne, Linum angustifolium, Linum catharticum, Linum flavum, Linum grandiflorum, Adenolinum grandiflorum, Linum lewisii, Linum narbonense, Linum perenne, Linum perenne var. lewisii, Linum pratense, Linum trigynum, Punica granatum, Gossypium hirsutum, Gossypium arboreum, Gossypium barbadense, Gossypium herbaceum, Gossypium thurberi, Musa nana, Musa acuminata, Musa paradisiaca, Musa spp., Elaeis guineensis, Papaver orientale, Papaver rhoeas, Papaver dubium, Sesamum indicum, Piper aduncum, Piper amalago, Piper angustifolium, Piper auritum, Piper betel, Piper cubeba, Piper longum, Piper nigrum, Piper retrofractum, Artanthe adunca, Artanthe elongata, Peperomia elongata, Piper elongatum, Steffensia elongata, Hordeum vulgare, Hordeum jubatum, Hordeum murinum, Hordeum secalinum, Hordeum distichon Hordeum aegiceras, Hordeum hexastichon., Hordeum hexastichum, Hordeum irregulare, Hordeum sativum, Hordeum secalinum, Avena sativa, Avena fatua, Avena byzantina, Avena fatua var. sativa, Avena hybrida, Sorghum bicolor, Sorghum halepense, Sorghum saccharatum, Sorghum vulgare, Andropogon drummondii, Holcus bicolor, Holcus sorghum, Sorghum aethiopicum, Sorghum arundinaceum, Sorghum caffrorum, Sorghum cernuum, Sorghum dochna, Sorghum drummondii, Sorghum durra, Sorghum guineense, Sorghum lanceolatum, Sorghum nervosum, Sorghum saccharatum, Sorghum subglabrescens, Sorghum verticilliflorum, Sorghum vulgare, Holcus halepensis, Sorghum miliaceum millet, Panicum militaceum, Zea mays, Triticum aestivum, Triticum durum, Triticum turgidum, Triticum hybernum, Triticum macha, Triticum sativum or Triticum vulgare, Cofea spp., Coffea arabica, Coffea canephora, Coffea liberica, Capsicum annuum, Capsicum annuum var. glabriusculum, Capsicum frutescens, Capsicum annuum, Nicotiana tabacum, Solanum tuberosum, Solanum melongena, Lycopersicon esculentum, Lycopersicon lycopersicum., Lycopersicon pyriforme, Solanum integrifolium, Solanum lycopersicum Theobroma cacao, Panicum virgatum or Camellia sinensis.

In another embodiment, the transgenic plant belongs to monocotyledonous or dicotyledonous plants, preferably to Anacardiaceae such as the genera Pistacia, Mangifera, Anacardium e.g. the species Pistacia vera [pistachios, Pistazie], Mangifer indica [Mango] or Anacardium occidentale [Cashew]; Asteraceae such as the genera Calendula, Carthamus, Centaurea, Cichorium, Cynara, Helianthus, Lactuca, Locusta, Tagetes, Valeriana e.g. the species Calendula officinalis [Marigold], Carthamus tinctorius [safflower], Centaurea cyanus [cornflower], Cichorium intybus [blue daisy], Cynara scolymus [Artichoke], Helianthus annus [sunflower], Lactuca sativa, Lactuca crispa, Lactuca esculenta, Lactuca scariola L. ssp. sativa, Lactuca scariola L. var. integrate, Lactuca scariola L. var. integrifolia, Lactuca sativa subsp. romana, Locusta communis, Valeriana locusta [lettuce], Tagetes lucida, Tagetes erecta or Tagetes tenuifolia [Marigold]; Apiaceae such as the genera Daucus e.g. the species Daucus carota [carrot]; Betulaceae such as the genera Corylus e.g. the species Corylus avellana or Corylus colurna [hazelnut]; Boraginaceae such as the genera Borago e.g. the species Borago officinalis [borage]; Brassicaceae such as the genera Brassica, Melanosinapis, Sinapis, Arabadopsis e.g. the species Brassica napus, Brassica rapa ssp. [canola, oilseed rape, turnip rape], Sinapis arvensis Brassica juncea, Brassica juncea var. juncea, Brassica juncea var. crispifolia, Brassica juncea var. foliosa, Brassica nigra, Brassica sinapioides, Melanosinapis communis [mustard], Brassica oleracea [fodder beet] or Arabidopsis thaliana; Bromeliaceae such as the genera Anana, Bromelia e.g. the species Anana comosus, Ananas ananas or Bromelia comosa [pineapple]; Caricaceae such as the genera Carica e.g. the species Carica papaya [papaya]; Cannabaceae such as the genera Cannabis e.g. the species Cannabis sative [hemp], Convolvulaceae such as the genera Ipomea, Convolvulus e.g. the species Ipomoea batatus, Ipomoea pandurata, Convolvulus batatas, Convolvulus tiliaceus, Ipomoea fastigiata, Ipomoea tiliacea, Ipomoea triloba or Convolvulus panduratus [sweet potato, Man of the Earth, wild potato], Chenopodiaceae such as the genera Beta, i.e. the species Beta vulgaris, Beta vulgaris var. altissima, Beta vulgaris var. Vulgaris, Beta maritima, Beta vulgaris var. perennis, Beta vulgaris var. conditiva or Beta vulgaris var. esculenta [sugar beet]; Cucurbitaceae such as the genera Cucubita e.g. the species Cucurbita maxima, Cucurbita mixta, Cucurbita pepo or Cucurbita moschata [pumpkin, squash]; Elaeagnaceae such as the genera Elaeagnus e.g. the species Olea europaea [olive]; Ericaceae such as the genera Kalmia e.g. the species Kalmia latifolia, Kalmia angustifolia, Kalmia microphylla, Kalmia polifolia, Kalmia occidentalis, Cistus chamaerhodendros or Kalmia lucida [American laurel, broad-leafed laurel, calico bush, spoon wood, sheep laurel, alpine laurel, bog laurel, western bog-laurel, swamp-laurel]; Euphorbiaceae such as the genera Manihot, Janipha, Jatropha, Ricinus e.g. the species Manihot utilissima, Janipha manihot, Jatropha manihot, Manihot aipil, Manihot dulcis, Manihot manihot, Manihot melanobasis, Manihot esculenta [manihot, arrowroot, tapioca, cassava] or Ricinus communis [castor bean, Castor Oil Bush, Castor Oil Plant, Palma Christi, Wonder Tree]; Fabaceae such as the genera Pisum, Albizia, Cathormion, Feuillea, Inga, Pithecolobium, Acacia, Mimosa, Medicajo, Glycine, Dolichos, Phaseolus, Soja e.g. the species Pisum sativum, Pisum arvense, Pisum humile [pea], Albizia berteriana, Albizia julibrissin, Albizia lebbeck, Acacia berteriana, Acacia littoralis, Albizia berteriana, Albizzia berteriana, Cathormion berteriana, Feuillea berteriana, Inga fragrans, Pithecellobium berterianum, Pithecellobium fragrans, Pithecolobium berterianum, Pseudalbizzia berteriana, Acacia julibrissin, Acacia nemu, Albizia nemu, Feuilleea julibrissin, Mimosa julibrissin, Mimosa speciosa, Sericanrda julibrissin, Acacia lebbeck, Acacia macrophylla, Albizia lebbek, Feuilleea lebbeck, Mimosa lebbeck, Mimosa speciosa [bastard logwood, silk tree, East Indian Walnut], Medicago sativa, Medicago falcata, Medicago varia [alfalfa], Glycine max [soybean], Dolichos soja, Glycine gracilis, Glycine hispida, Phaseolus max or Soja hispida; Geraniaceae such as the genera Pelargonium, Cocos, Oleum e.g. the species Cocos nucifera, Pelargonium grossularioides or Oleum cocois [coconut]; Gramineae such as the genera Saccharum e.g. the species Saccharum officinarum; Juglandaceae such as the genera Juglans, Wallia e.g. the species Juglans regia, Juglans ailanthifolia, Juglans sieboldiana, Juglans cinerea, Wallia cinerea, Juglans bixbyi, Juglans californica, Juglans hindsii, Juglans intermedia, Juglans jamaicensis, Juglans major, Juglans microcarpa, Juglans nigra or Wallia nigra [walnut, black walnut, common walnut, persian walnut, white walnut, butternut, black walnut]; Lauraceae such as the genera Persea, Laurus e.g. the species laurel Laurus nobilis [bay, laurel, bay laurel, sweet bay], Persea americana Persea americana, Persea gratissima or Persea persea [avocado]; Leguminosae such as the genera Arachis e.g. the species Arachis hypogaea [peanut]; Linaceae such as the genera Linum, Adenolinum e.g. the species Linum usitatissimum, Linum humile, Linum austriacum, Linum bienne, Linum angustifolium, Linum catharticum, Linum flavum, Linum grandiflorum, Adenolinum grandiflorum, Linum lewisii, Linum narbonense, Linum perenne, Linum perenne var. lewisii, Linum pratense or Linum trigynum [flax, linseed]; Lythrarieae such as the genera Punica e.g. the species Punica granatum [pomegranate]; Malvaceae such as the genera Gossypium e.g. the species Gossypium hirsutum, Gossypium arboreum, Gossypium barbadense, Gossypium herbaceum or Gossypium thurberi [cotton]; Musaceae such as the genera Musa e.g. the species Musa nana, Musa acuminata, Musa paradisiaca, Musa spp. [banana]; Onagraceae such as the genera Camissonia, Oenothera e.g. the species Oenothera biennis or Camissonia brevipes [primrose, evening primrose]; Palmae such as the genera Elacis e.g. the species Elaeis guineensis [oil plam]; Papaveraceae such as the genera Papaver e.g. the species Papaver orientale, Papaver rhoeas, Papaver dubium [poppy, oriental poppy, corn poppy, field poppy, shirley poppies, field poppy, long-headed poppy, long-pod poppy]; Pedaliaceae such as the genera Sesamum e.g. the species Sesamum indicum [sesame]; Piperaceae such as the genera Piper, Artanthe, Peperomia, Steffensia e.g. the speciesPiper aduncum, Piper amalago, Piper angustifolium, Piper auritum, Piper betel, Piper cubeba, Piper longum, Piper nigrum, Piper retrofractum, Artanthe adunca, Artanthe elongata, Peperomia elongata, Piper elongatum, Steffensia elongata. [Cayenne pepper, wild pepper]; Poaceae such as the genera Hordeum, Secale, Avena, Sorghum, Andropogon, Holcus, Panicum, Oryza, Zea, Triticum e.g. the species Hordeum vulgare, Hordeum jubatum, Hordeum murinum, Hordeum secalinum, Hordeum distichon Hordeum aegiceras, Hordeum hexastichon, Hordeum hexastichum, Hordeum irregulare, Hordeum sativum, Hordeum secalinum [barley, pearl barley, foxtail barley, wall barley, meadow barley], Secale cereale [rye], Avena sativa, Avena fatua, Avena byzantina, Avena fatua var. sativa, Avena hybrida [oat], Sorghum bicolor, Sorghum halepense, Sorghum saccharatum, Sorghum vulgare, Andropogon drummondii, Holcus bicolor, Holcus sorghum, Sorghum aethiopicum, Sorghum arundinaceum, Sorghum caffrorum, Sorghum cernuum, Sorghum dochna, Sorghum drummondii, Sorghum durra, Sorghum guineense, Sorghum lanceolatum, Sorghum nervosum, Sorghum saccharatum, Sorghum subglabrescens, Sorghum verticilliflorum, Sorghum vulgare, Holcus halepensis, Sorghum miliaceum millet, Panicum militaceum [Sorghum, millet], Oryza sativa, Oryza latifolia [rice], Zea mays [corn, maize] Triticum aestivum, Triticum durum, Triticum turgidum, Triticum hybernum, Triticum macha, Triticum sativum or Triticum vulgare [wheat, bread wheat, common wheat], Proteaceae such as the genera Macadamia e.g. the species Macadamia intergrifolia [macadamia]; Rubiaceae such as the genera Coffea e.g. the species Cofea spp., Coffea arabica, Coffea canephora or Coffea liberica [coffee]; Scrophulariaceae such as the genera Verbascum e.g. the species Verbascum blattaria, Verbascum chaixii, Verbascum densiflorum, Verbascum lagurus, Verbascum longifolium, Verbascum lychnitis, Verbascum nigrum, Verbascum olympicum, Verbascum phlomoides, Verbascum phoenicum, Verbascum pulverulentum or Verbascum thapsus [mullein, white moth mullein, nettle-leaved mullein, dense-flowered mullein, silver mullein, long-leaved mullein, white mullein, dark mullein, greek mullein, orange mullein, purple mullein, hoary mullein, great mullein]; Solanaceae such as the genera Capsicum, Nicotiana, Solanum, Lycopersicon e.g. the species Capsicum annuum, Capsicum annuum var. glabriusculum, Capsicum frutescens [pepper], Capsicum annuum [paprika], Nicotiana tabacum, Nicotiana alata, Nicotiana attenuata, Nicotiana glauca, Nicotiana langsdorffii, Nicotiana obtusifolia, Nicotiana quadrivalvis, Nicotiana repanda, Nicotiana rustica, Nicotiana sylvestris [tobacco], Solanum tuberosum [potato], Solanum melongena [egg-plant] (Lycopersicon esculentum, Lycopersicon lycopersicum, Lycopersicon pyriforme, Solanum integrifolium or Solanum lycopersicum [tomato]; Sterculiaceae such as the genera Theobroma e.g. the species Theobroma cacao [cacao]; Theaceae such as the genera Camellia e.g. the species Camellia sinensis) [tea].

In another embodiment of the invention the transgenic non-human organism is a transgenic microorganism like selected from the group consisting of Charophyceae such as the genera Chara, Nitella e.g. the species Chara globularis, Chara vulgaris, Nitella flexilis, Chlorophyceae such as the genera Acrosiphonia, Spongomorpha, Urospora, Bryopsis, Pseudobryopsis, Trichosolen, Dichotomosiphon, Caulerpa, Rhipilia, Blastophysa, Avrainvillea, Chlorodesmis, Codium, Espera, Halicystis, Halimeda, Penicillus, Pseudocodium, Rhipiliopsis, Rhipocephalus, Tydemania, Udotea, Derbesia, Acrochaete, Aphanochaete, Bolbocoleon, Chaetobolus, Chaetonema, Chaetophora, Chlorotylium, Desmococcus, Draparnaldia, Draparnaldiopsis, Ectochaete, Endophyton, Entocladia, Epicladia, Internoretia, Microthamnion, Ochlochaete, Phaeophila, Pilinella, Pringsheimiella, Protoderma, Pseudendoclonium, Pseudodictyon, Pseudopringsheimia, Pseudulvella, Schizomeris, Stigeoclonium, Thamniochaete, Ulvella, Pilinia, Tellamia, Helicodictyon, Actidesmium, Ankyra, Characium, Codiolum, Sykidion, Keratococcus, Prototheca, Bracteacoccus Chlorococcum, Excentrosphaera, Hormidium, Oophila,

Schroederia, Tetraedron, Trebouxia Chlorosarcinopsis, Gomphonitzschia, Coccomyxa, Dactylothece, Diógenes, Disporá, Gloeocystis, Mycanthococcus, Ourococcus, Coelastrum, Dicranochaete, Botryococcus, Dictyosphaerium, Dimorphococcus, Chlorochytrium, Kentrosphaera, Phyllobium, Gomontia, Hormotila, Euastropsis, Hydrodictyon, Pectodictyon, Pediastrum, Sorastrum, Tetrapedia, Acanthosphaera, Echinosphaerella, Echinosphaeridium, Errerella, Gloeoactinium, Golenkeniopsis Golenkinia, Micractinium, Ankistrodesmus, Chlorella, Chodatella, Closteriopsis, Cryocystis, Dactylococcus, Dematractum, Eremosphaera, Eutetramorus, Franceia, Glaucocystis, Gloeotaenium, Kirchneriella, Lagerheimiella, Monoraphidium, Nannochloris, Nephrochlamys, Nephrocytium, Oocystis, Oonephris, Pachycladon, Palmellococcus, Planktosphaeria, Polyedriopsis, Pseudoraciborskia, Quadrigula, Radiococcus, Rochiscia, Scotiella, Selanastrum, Thorakochloris, Treubaria, Trochiscia, Westella, Zoochlorella, Ostreobium, Phyllosiphon, Protosiphon, Rhodochytrium, Actinastrum, Coronastrum, Crucigenia, Dictymocystis, Enallax, Scenedesmus, Selenastrum, Tetradesmus, Tetrallantos, Tetrastrum, Chlorosarcina, Anadyomene, Valoniopsis, Ventricaria, Basicladia, Chaetomorpha, Cladophora, LolaPithophoraRhizoclonium Chaetosphaeridium, Conochaete, Coleochaete, Oligochaetophora, Polychaetophora, Cylindrocapsa, Gongrosira, Protococcus, Acetabularia, Batophora, Bornetella, Dasycladus, Halicoryne, Neomeris, Elakatothrix, Raphidonema, Microspora, Bulbochaete, Oedocladium, Oedogonium, Prasiola, Rosenvingiella, Schizogonium, Apjohnia, Chamaedoris, Cladophoropsis, Siphonocladus, Spongocladia, Boergesenia, Boodlea, Cystodictyon, Dictyosphaeria, Ernodesmis, Microdictyon, Struvea, Valonia, Sphaeroplea, Malleochloris, Stylosphaeridium, Gloeococcus, Palmella, Palmodictyon, Palmophyllum, Pseudospherocystis, Sphaerocystis, Urococcus, Apiocystis, Chaetopeltis, Gemellicystis, Paulschulzia, Phacomyxa, Pseudotetraspora, Schizochlamys, Tetraspora, Cephaleuros, Ctenocladus, Epibolium, Leptosira, Trentepohlia, Diplochaete, Monostroma, Binuclearia, Geminella, Klebsormidium, Planetonema, Radiofilum, Stichococcus, Ulothrix, Uronema, Blidingia, Capsosiphon, Chloropelta, Enteromorpha, Percursaria, Ulva, Ulvaria, Brachiomonas, Carteria, Chlainomonas, Chlamydomonas, Chlamydonephris, Chlorangium, Chlorogonium, Cyanidium, Fortiella, Glenomonas, Gloeomonas, Hyalogonium, Lobomonas Polytoma, Pyramichlamys, Scourfieldia, Smithsonimonas, Sphaerellopsis, Sphenochloris, Spirogonium, Collodictyon, Dunaliella, Haematococcus, Stephanosphaera, Coccomonas, Dysmorphococcus, Phacotus, Pteromonas, Thoracomonas, Wislouchiella, Mascherina, Pyrobotrys, Spondylomorum, Eudorina, Gonium, Oltmannsiella, Pandorina, Platydorina, Pleodorina, Stephanoon, Volvox, Volvulina, Actinotaenium, Arthrodesmus, Bambusina Closterium, Cosmarium, Desmidium, Euastrum, Groenbladia, Hyalotheca, Micrasterias, Penium, Phymatodocis, Pleurotaenium, Sphaerozosma, Spinoclosterium, Spinocosmarium, Spondylosium, Staurastrum, Tetmemorus, Triploceras, Xanthidium, Cylindrocystis, Genicularia, Gonatozygon, Mesotaenium, Netrium, Roya, Spirotaenia, Cosmocladium, Debarya, Docidium, Euastridium, Hallasia, Mougeotia, Mougeotiopsis, Sirogonium, Spirogyra, Staurodesmus, Teilingia, Zygnema, Zygogonium, e.g. the species Caulerpa taxifolia, Prototheca wickerhamii, Ankistrodesmus falcatus, Chlorella ellipsoidea, Chlorella pyrenoidosa, CloreIla sorokiniana, Chlorella vulgaris, Scenedesmus obliquus, Scenedesmus quadricauda, Selenastrum capricornutum, Selenastrum undecimnotata, Cladophora glomerata, Chlamydomonas eugametos, Chlamydomonas reinhardtii, Cyanidium caldarium, Dunaliella salina, Dunaliella tertiolecta, Euglena gracilis, Haematococcus pluvialis, Coniugatophyceae, Prasinophyceae Trebouxiophyceae, Ulvophyceae, Chlorodendraceae, Pedinomonadales, Halosphaeraceae, Pterospermataceae, Monomastigaceae, Pyramimonadaceae, Chlorodendraceae such as the genera Prasinocladus e.g. the species Prasinocladus ascus, Halosphaeraceae, Pedinomonadales, Pedinomonadaceae such as the genera Pedinomonas, Pterospermataceae such as the genera Pachysphaera, Pterosperma, Halosphaera, Pyramimonas, Bacillariophyceae, Chrysophyceae, Craspedophyceae, Euglenophyceae, Prymnesiophyceae, Phaeophyceae, Dinophyceae, Rhodophyceae, Xanthophyceae, Prasinophyceae such as the genera Nephroselmis, Prasinococcus, Scherffelia, Tetraselmis, Mantoniella, Ostreococcus e.g. the species Nephroselmis olivacea, Prasinococcus capsulatus, Scherffelia dubia, Tetraselmis chui, Tetraselmis suecica, Mantoniella squamata or Ostreococcus tauri.

Other useful organism are prokaryotic host organism, which may be useful for the cloning of the desired nucleic acid constructs or vectors such as the genera Escherichia, for example the species Escherichia coli, in particular E. coli K12 and its described strains or Agrobacterium, for example Agrrobacterium tumefaciens.

The introduction of the nucleic acids according to the invention, the expression cassette or the vector into the non-human organism, like a microorganism or a plant, can in principle be done by all of the methods known to those skilled in the art. The introduction of the nucleic acid sequences gives rise to recombinant or transgenic organisms.

After the introduction of the nucleic acid, nucleic acid construct, expression cassette or vector, and the expression thereof the transgenic organism or cell is advantageously cultured and subsequently harvested.

Unless otherwise specified, the terms “polynucleotides”, “nucleic acid” and “nucleic acid molecule” as used herein are interchangeably. Unless otherwise specified, the terms “peptide”, “polypeptide” and “protein” are interchangeably in the present context. The term “sequence” may relate to polynucleotides, nucleic acids, nucleic acid molecules, peptides, polypeptides and proteins, depending on the context in which the term “sequence” is used. The terms “gene(s)”, “polynucleotide”, “nucleic acid sequence”, “nucleotide sequence”, or “nucleic acid molecule(s)” as used herein refers to a polymeric form of nucleotides of any length, either ribonucleotides or deoxyribonucleotides. The terms refer only to the primary structure of the molecule.

Thus, the terms “gene(s)”, “polynucleotide”, “nucleic acid sequence”, “nucleotide sequence”, or “nucleic acid molecule(s)” as used herein include double- and singles-stranded DNA and RNA. They also include known types of modifications, for example, methylation, “caps”, substitutions of one or more of the naturally occurring nucleotides with an analog. Preferably, the DNA or RNA sequence of the invention comprises a coding sequence encoding the herein defined polypeptide.

The genes of the invention, coding for an activity selected from the group consisting of (DL)-glycerol-3-phosphatase, 2,3-dihydroxyphenylpropionate 1,2-dioxygenase, 2-oxoglutarate dehydrogenase E1 subunit, 49747384_SOYBEAN-protein, 5′-nucleotidase, acetolactate synthase small subunit, acetyl CoA carboxylase, adenosine kinase, arginine exporter protein, At1g09680-protein, At2g45420-protein, At4g32480-protein, ATP-binding component of a transport system, auxin response factor, b0012-protein, b1003-protein, b1522-protein, b2032-protein, b2345-protein, b2513-protein, b2673-protein, b3246-protein, b3346-protein, b3817-protein, b4029-protein, beta-hydroxylase, bifunctional aspartokinase/homoserine dehydrogenase, calcium-dependent protein kinase, coproporphyrinogen III oxidase, cyclin, cystathionine gamma-synthase, cystathionine-lyase, dihydroxyacid dehydratase, DNA-binding protein, F-box protein, glutaredoxin, glutathione S-transferase, glycogenin, homocitrate synthase, homoserine dehydrogenase, hydrolase, L-serine dehydratase, major facilitator superfamily transporter protein, malic enzyme, membrane transport protein, monothiol glutaredoxin, monthiol glutaredoxin, oxidoreductase, peptidyl-prolyl cis-trans isomerase, phosphoadenosine phosphosulfate reductase , Photosystem I reaction center subunit XI, protein kinase, protein phosphatase, pyruvate kinase, Sec-independent protein translocase subunit, serine protease, serine/threonineprotein phosphatase, threonine aldolase, threonine dehydratase, threonine efflux protein, transcription factor, transport protein, uridine/cytidine kinase, valine-pyruvate transaminase, yhl013c-protein, yml084w-protein, yol160w-protein, yor392w-protein, and zinc finger protein are also called “FCRP genes”.

A “coding sequence” is a nucleotide sequence, which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. The triplets taa, tga and tag represent the (usual) stop codons which are interchangeable. A coding sequence can include, but is not limited to mRNA, cDNA, recombinant nucleotide sequences or genomic DNA, while introns may be present as well under certain circumstances.

To introduce a nucleic acid molecule into a nucleic acid construct, e.g. as part of an expression cassette, the codogenic gene segment is advantageously subjected to an amplification and ligation reaction in the manner known by a skilled person. It is preferred to follow a procedure similar to the protocol for the Pfu DNA polymerase or a Pfu/Taq DNA polymerase mixture. The primers are selected according to the sequence to be amplified. The primers should expediently be chosen in such a way that the amplificate comprise the codogenic sequence from the start to the stop codon. After the amplification, the amplificate is expediently analyzed. For example, the analysis may consider quality and quantity and be carried out following separation by gel electrophoresis. Thereafter, the amplificate can be purified following a standard protocol (for example Qiagen). An aliquot of the purified amplificate is then available for the subsequent cloning step. The skilled worker generally knows suitable cloning vectors.

They include, in particular, vectors which are capable of replication in easy to handle cloning systems like as bacterial, yeast or insect cell based (e.g. baculovirus expression) systems, that is to say especially vectors which ensure efficient cloning in E. coli, and which make possible the stable transformation of plants. Vectors, which must be mentioned, in particular are various binary and cointegrated vector systems, which are suitable for the T-DNA-mediated transformation. Such vector systems are generally characterized in that they contain at least the vir genes, which are required for the Agrobacterium-mediated transformation, and the T-DNA border sequences.

In general, vector systems preferably also comprise further cisregulatory regions such as promoters and terminators and/or selection markers by means of which suitably transformed organisms can be identified. While vir genes and T-DNA sequences are located on the same vector in the case of cointegrated vector systems, binary systems are based on at least two vectors, one of which bears vir genes, but no T-DNA, while a second one bears T-DNA, but no vir gene. Owing to this fact, the last-mentioned vectors are relatively small, easy to manipulate and capable of replication in E. coli and in Agrobacterium. These binary vectors include vectors from the series pBIB-HYG, pPZP, pBecks, pGreen. Those which are preferably used in accordance with the invention are Bin19, pBI101, pBinAR, pGPTV and pCAMBIA. An overview of binary vectors and their use is given by Hellens et al, Trends in Plant Science 5, 446-451 (2000). In case of a targeted expression the vectors are preferably modified in such a manner, that they already contain the nucleic acid coding for the transit peptide and that the nuleic acids of the invention, preferentially the nucleic acid sequences encoding the polypeptides shown in the respective line in Table II, column 5 or 8, or a homolog or a fragment thereof, can be cloned 3″prime to the transit peptide encoding sequence, leading to a functional preprotein, which is directed to the intended compartment, like plastids or mitochondria, and which means that the mature protein fulfills its biological activity.

For a vector preparation, vectors may first be linearized using restriction endonuclease(s) and then be modified enzymatically in a suitable manner. Thereafter, the vector is purified, and an aliquot is employed in the cloning step. In the cloning step, the enzyme-cleaved and, if required, purified amplificate is cloned together with similarly prepared vector fragments, using ligase. In this context, a specific nucleic acid construct, or vector or plasmid construct, may have one or else more codogenic gene segments. The codogenic gene segments in these constructs are preferably linked operably to regulatory sequences. The regulatory sequences include, in particular, plant sequences like the above-described promoters and terminators. The constructs can advantageously be propagated stably in microorganisms, in particular Escherichia coli and/or Agrobacterium tumefaciens, under selective conditions and enable the transfer of homologous or heterologous DNA into non-human organisms, like plants or other microorganisms. In accordance with a particular embodiment, the constructs are based on binary vectors (overview of a binary vector: Hellens et al., 2000). As a rule, they contain prokaryotic regulatory sequences, such as replication origin and selection markers, for the multiplication in microorganisms such as Escherichia coli and Agrobacterium tumefaciens. Vectors can further contain agrobacterial T-DNA sequences for the transfer of DNA into plant genomes or other eukaryotic regulatory sequences for transfer into other eukaryotic cells, e.g. Saccharomyces sp. or other prokaryotic regulatory sequences for the transfer into other prokaryotic cells, e.g. Corynebacterium sp. or Bacillus sp. For the transformation of plants, the right border sequence, which comprises approximately 25 base pairs, of the total agrobacterial T-DNA sequence is advantageously included. Usually, the plant transformation vector constructs according to the invention contain T-DNA sequences both from the right and from the left border region, which contain expedient recognition sites for site-specific acting enzymes, which, in turn, are encoded by some of the vir genes.

Alternatively the nuleic acids of the invention are cloned into vectors, which are designed for the direct transformation of organelles such as for example plastids. Generally such vectors additionally carry a specific resistance gene (as mentioned above), like the spectomycin resistance gene (aad) under control of a plastid regulatory sequence and two adjacent plastome sequences of the target organism, which mediated the directed insertion of the sequences of interest, e.g. the resistance gene and the expression cassette, into the plastidal genome through homologous recombination. As transformation can be achieved by particle bombardment or other physical or chemical methods e.g. PEG treatment or microinjection, the vectors do not need to contain the elements necessary for agrobacterial T-DNA transfer (see below).

In order to introduce, into a plant, the nucleic acid molecule of the invention or used in the process according to the invention, it has proved advantageous first to transfer them into an intermediate host, for example a bacterium or a eukaryotic unicellular cell. The transformation into E. coli, which can be carried out in a manner known per se, for example by means of heat shock or electroporation, has proved itself expedient in this context. Thus, the transformed E. coli colonies can be analysed for their cloning efficiency. This can be carried out with the aid of a PCR. Here, not only the identity, but also the integrity, of the plasmid construct can be verified with the aid of a defined colony number by subjecting an aliquot of the colonies to said PCR. As a rule, universal primers which are derived from vector sequences are used for this purpose, it being possible, for example, for a forward primer to be arranged upstream of the start ATG and a reverse primer to be arranged downstream of the stop codon of the codogenic gene segment. The amplificates are separated by electrophoresis and assessed with regard to quantity and quality.

The nucleic acid constructs, which are optionally verified, are subsequently used for the transformation of the plants or other hosts, e.g. other eukaryotic cells or other prokaryotic cells. To this end, it may first be necessary to obtain the constructs from the intermediate host. For example, the constructs may be obtained as plasmids from bacterial hosts by a method similar to conventional plasmid isolation.

The nucleic acid molecule of the invention or used in the process according to the invention can also be introduced into modified viral vectors like baculovirus vectors for expression in insect cells or plant viral vectors like tobacco mosaic virus or potato virus X-based vectors. Approaches leading to the expression of proteins from the modified viral genome including the nucleic acid molecule of the invention or used in the process according to the invention involve for example the inoculation of tobacco plants with infectious RNA transcribed in vitro from a cDNA copy of the recombinant viral genome. Another approach utilizes the transfection of whole plants from wounds inoculated with Agrobacterium tumefaciens containing cDNA copies of recombinant plus-sense RNA viruses. Different vectors and virus are known to the skilled worker for expression in different target e.g. production plants.

A large number of methods for the transformation of plants are known. Since, in accordance with the invention, a stable integration of heterologous or additional homologous DNA into the genome of plants is advantageous, the T-DNA-mediated transformation has proved expedient in particular. For this purpose, it is first necessary to transform suitable vehicles, in particular agrobacteria, with a codogenic gene segment or the corresponding plasmid construct comprising the nucleic acid molecule of the invention or an expression cassette according to the invention. This can be carried out in a manner known per se. For example, said nucleic acid construct of the invention, like said expression cassette or said plasmid construct, which has been generated in accordance with what has been detailed above, can be transformed into competent agrobacteria by means of electroporation or heat shock. In principle, one must differentiate between the formation of cointegrated vectors on the one hand and the transformation with binary vectors on the other hand. In the case of the first alternative, the constructs, which comprise the codogenic gene segment or the nucleic acid molecule of the invention have no T-DNA sequences, but the formation of the cointegrated vectors or constructs takes place in the agrobacteria by homologous recombination of the construct with T-DNA. The T-DNA is present in the agrobacteria in the form of Ti or Ri plasmids in which exogenous DNA has expediently replaced the oncogenes. If binary vectors are used, they can be transferred to agrobacteria either by bacterial conjugation or by direct transfer. These agrobacteria expediently already comprise the vector bearing the vir genes (currently referred to as helper Ti (Ri) plasmid). As mentioned before the stable integration of the heterologous (or additional homologous) nucleic acids into the plastidial genome may also be advantegously.

One or more markers may expediently also be used together with the nucleic acid construct, like the expression cassette, or the vector of the invention and, if plants or plant cells shall be transformed together with the T-DNA, with the aid of which the isolation or selection of transformed organisms, such as agrobacteria or transformed plant cells, is possible. These marker genes enable the identification of a successful transfer of the nucleic acid molecules according to the invention via a series of different principles, for example via visual identification with the aid of fluorescence, luminescence or in the wavelength range of light which is discernible for the human eye, by a resistance to herbicides or antibiotics, via what are known as nutritive markers (auxotrophism markers) or antinutritive markers, via enzyme assays or via phytohormones. Examples of such markers which may be mentioned are GFP (=green fluorescent protein); the luciferin/luceferase system, the β3-galactosidase with its colored substrates, for example X-Gal, the herbicide resistances to, for example, imidazolinone, glyphosate, phosphinothricin or sulfonylurea, the antibiotic resistances to, for example, bleomycin, hygromycin, streptomycin, kanamycin, tetracyclin, chloramphenicol, ampicillin, gentamycin, geneticin (G418), spectinomycin or blasticidin, to mention only a few, nutritive markers such as the utilization of mannose or xylose, or antinutritive markers such as the resistance to 2-deoxyglucose. This list is a small number of possible markers. The skilled worker is very familiar with such markers. Different markers are preferred, depending on the organism and the selection method. In case of plastidal transformation methods other marker genes known to a person skilled in the art may be used, but also the ones mentioned above, preferably e.g. the spectomycin resistance gene (aadA).

As a rule, it is desired that the plant nucleic acid constructs, plant expression cassettes, are flanked by T-DNA at one or both sides of the codogenic gene segment. This is particularly useful when bacteria of the species Agrobacterium tumefaciens or Agrobacterium rhizogenes are used for the transformation. A method, which is preferred in accordance with the invention, is the transformation with the aid of Agrobacterium tumefaciens. However, biolistic methods may also be used advantageously for introducing the sequences in the process according to the invention, and the introduction by means of PEG is also possible. The transformed agrobacteria can be grown in the manner known per se and are thus available for the expedient transformation of the plants. The plants or plant parts to be transformed are grown or provided in the customary manner. The transformed agrobacteria are subsequently allowed to act on the plants or plant parts until a sufficient transformation rate is reached. Allowing the agrobacteria to act on the plants or plant parts can take different forms. For example, a culture of morphogenic plant cells or tissue may be used. After the T-DNA transfer, the bacteria are, as a rule, eliminated by antibiotics, and the regeneration of plant tissue is induced. This is done in particular using suitable plant hormones in order to initially induce callus formation and then to promote shoot development.

Plant cells, plant tissues etc. may be transformed transient or stable. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least the flower primordia. The plant is subsequently grown on until the seeds of the treated plant are obtained (Clough and Bent, Plant J. 16, 735 (1998)). To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Further advantageous transformation methods, in particular for plants, are known to the skilled worker and are described herein.

Further advantageous and suitable methods are protoplast transformation by poly(ethylene glycol)-induced DNA uptake, the “biolistic” method using the gene cannon—referred to as the particle bombardment method, electroporation, the incubation of dry embryos in DNA solution, microinjection and gene transfer mediated by Agrobacterium. Said methods are described by way of example in Jenes B. et al., Techniques for Gene Transfer, in: Transgenic Plants, Vol. 1, Engineering and Utilization, eds. Kung S.D. and Wu R., Academic Press (1993) 128-143 and in Potrykus, Annu. Rev. Plant Physiol. Plant Molec. Biol. 42, 205 (1991)). The nucleic acids or the construct, e.g. the expression cassette, to be expressed is preferably cloned into a vector, which is suitable for transforming Agrobacterium tumefaciens, for example pBin19 (Bevan et al., Nucl. Acids Res. 12, 8711 (1984)). Agrobacteria transformed by an expression vector according to the invention may likewise be used in known manner for the transformation of plants such as test plants like Arabidopsis or crop plants such as cereal crops, corn, oats, rye, barley, wheat, soybean, rice, cotton, sugar beet, canola, sunflower, flax, hemp, potatoes, tobacco, tomatoes, carrots, paprika, oilseed rape, tapioca, cassava, arrowroot, tagetes, alfalfa, lettuce and the various tree, nut and vine species, in particular oil-containing crop plants such as soybean, peanut, castor oil plant, sunflower, corn, cotton, flax, oilseed rape, coconut, oil palm, safflower (Carthamus tinctorius) or cocoa bean, or in particular corn, wheat, soybean, rice, cotton and canola, e.g. by bathing bruised leaves or chopped leaves in an agrobacterial solution and then culturing them in suitable media. The transformation of plants by means of Agrobacterium tumefaciens is described, for example, by Hofgen and Willmitzer in Nucl. Acid Res. 16, 9877 (1988) or is known inter alia from White F. F., “Vectors for Gene Transfer in Higher Plants”; in Transgenic Plants, Vol. 1, Engineering and Utilization, eds. Kung S. D. and Wu R., Academic Press, 1993, pp. 15-38. For the transformation of plastids physical methods like PEG-treatment (O'Neil et al., Plant Journal. 3, 729 (1993), Golds et al., BioTechnology 11, 95 (1993)), microinjection (Knoblauch et al., Nat. Biotech. 17, 906 (1999)) or biolistics (Svab et al., Proc. Natl. Acad. Sci. USA 90, 8526 (1990)) are preferred. Such transformation methods are especially useful for the direct transformation of plastids and are well known to the skilled worker.

The expression of the nucleic acid molecules used in the process according the present invention may be desired alone or in combination with other genes or nucleic acid molecules. Multiple nucleic acid molecules conferring the expression of advantageous genes can be introduced via the simultaneous transformation of several individual suitable nucleic acid constructs, i.e. expression constructs, or, preferably, by combining several expression cassettes on one construct. It is also possible to transform the recipient non-human organisms stepwise with several vectors which in each case comprises a single expression cassette or several expression cassettes.

In addition to the sequence mentioned in Table I, application no. 1, column 5 or 8, preferably the coding region thereof, or its homologs or fragments, it may be advantageous additionally to express and/or mutate further genes in the non-human organisms. Especially advantageously, additionally at least one further gene of the biosynthetic pathway of the fine chemical is expressed in the non-human organisms such as plants or microorganisms. It is also possible that the regulation of the natural genes has been modified advantageously so that the gene and/or its gene product is no longer subject to the regulatory mechanisms which exist in the non-human organisms. This leads to an increased synthesis of the respective amino acid since, for example, feedback regulations no longer exist to the same extent or not at all. In addition it might be advantageously to combine the nucleic acids sequences of the invention containing the sequences shown in the respective line in Table I, application no. 1, column 5 or 8, preferably the coding region thereof, or homologs or fragments thereof, with genes which generally support or enhance the growth or yield of the target non-human organisms, for example genes which lead to faster growth rate of non human organism like microorganisms or plants or genes which produce stress-, pathogen-, or herbicide-resistant plants.

In a further embodiment of the process of the invention, non-human organisms are grown, in which there is in addition to the expression of the nucleic acid molecule according to the invention simultaneous expression of at least one nucleic acid molecule or one of the genes which code for proteins involved in the amino acid metabolism, in particular in amino acid synthesis, especially genes coding for proteins selected from the group coding for aspartate kinase (lysC), aspartate-semialdehyde dehydrogenase (asd), glyceraldehyde-3-phosphate dehydrogenase (gap), 3-phosphoglycerate kinase (pgk), pyruvate carboxylase (pyc), triosephosphate isomerase (tpi), homoserine O-acetyltransferase (metA), cystathionine γ-synthase (metB), cystathionine gamma-lyase (metC), cystathionine β-lyase, methionine synthase (metH), serine hydroxymethyltransferase (glyA), O-acetyl homoserine sulfhydrylase (metY), methylenetetrahydrofolate reductase (metF), phosphoserine aminotransferase (serC), phosphoserine phosphatase (serB), serine acetyltransferase (cysE), cysteine synthase (cysK), homoserine dehydrogenase (hom) and S-adenosyL-methionine synthase (metX) in the cytosol or in an organelle, like plastids or mitochondria. A further advantageous nucleic acid sequence which can be expressed in combination with the nucleic acid sequences according to the invention and if desired, the above-mentioned biosynthesis genes, is the sequence of the ATP/ADP translocator as described in WO 01/20009. This ATP/ADP translocator leads to an increased synthesis of the essential amino acids lysine and/or methionine.

In a further advantageous embodiment of the process of the invention, the non-human organisms used in the process are those in which simultaneously in addition a fine chemical degrading protein is attenuated, in particular by reducing the rate of expression of the corresponding gene.

In another embodiment of the process of the invention, the non-human organisms used in the process are those in which simultaneously in addition at least one of the aforementioned nucleic acids or of the aforementioned genes is mutated in such a way that the enzymatic or biological activity of the corresponding fine chemical degrading protein is partially reduced or completely blocked. A reduction in the enzymatic or biological activity means an enzymatic or biological activity, which is reduced by at least 10%, advantageously at least 20%, 30%, 40%, 50%, 60%, 70% or more, compared with the starting organism.

If it is intended to transform the host cell, in particular the plant cell, with several constructs, expression cassettes or vectors, the marker of a preceding transformation must be removed or a further marker employed in a following transformation. The markers can be removed from the host cell, in particular the plant cell, as described herein below via methods with which the skilled worker is familiar. These methods may be not used for multiple transformed host cell only but also for single transformed host cells. In particular plants without a marker, in particular without resistance to antibiotics, are an especially preferred embodiment of the present invention.

In the process according to the invention, the nucleic acid molecules used in the process according to the invention are advantageously linked operably to one or more regulatory signals in order to increase gene expression. These regulatory sequences are intended to enable the specific expression of the genes and the expression of protein. Depending on the host organism for example plant or microorganism, this may mean, for example, that the gene is expressed and/or overexpressed after induction only, or that it is expressed and/or overexpressed constitutively. These regulatory sequences are, for example, sequences to which the inductors or repressors bind and which thus regulate the expression of the nucleic acid. In addition to these novel regulatory sequences, or instead of these sequences, the natural regulation of these sequences may still be present before the actual structural genes and, if appropriate, may have been genetically modified so that the natural regulation has been switched off and gene expression has been increased. However, the nucleic acid construct of the invention suitable as expression cassette (=expression construct =gene construct) can also be simpler in construction, that is to say no additional regulatory signals have been inserted before the nucleic acid molecule or the homolog or fragment thereof, and the natural promoter together with its regulation has not been removed. Instead, the natural regulatory sequence has been mutated in such a way that regulation no longer takes place and/or gene expression is increased. These modified promoters can also be introduced on their own before the natural gene in the form of part sequences (=promoter with parts of the nucleic acid sequences according to the invention) in order to increase the activity. Moreover, the gene construct can advantageously also comprise one or more of what are known as enhancer sequences in operable linkage with the promoter, and these enable an increased expression of the nucleic acid sequence. Also, it is possible to insert additional advantageous sequences at the 3′ end of the DNA sequences, such as, for example, further regulatory elements or terminators. In another preferred embodiment, the natural or created expression cassette is further modified in such a manner, that a nucleic acid sequence encoding a transit peptide is functionally introduced between the regulatory and the coding region such, that a functionally preprotein is expressed, which is targeted to the organelles such as plastids or mitochondria, preferably the plastids.

The nucleic acid molecules, which encode proteins according to the invention and nucleic acid molecules, which encode other polypeptides may be present in one nucleic acid construct, expression cassette or vector or in respective several ones. Advantageously, only one copy of the nucleic acid molecule of the invention or its encoding genes is present in the nucleic acid construct, expression cassette or vector. Several vectors, nucleic acid construct, or expression cassettes can be expressed together in the host organism. The nucleic acid molecule, the nucleic acid construct or the expression cassette according to the invention can be inserted in a vector and be present in the cell in a free form. If a stable transformation is preferred, a vector is used, which is stably duplicated over several generations or which is else inserted into the genome. In the case of plants, integration into the genome of e.g. plastids or mitochondria or, in particular, into the nuclear genome may have taken place. For the insertion of more than one gene in the host genome the genes to be expressed are present together in one gene construct, for example in above-described vectors bearing a plurality of genes.

As a rule, regulatory sequences for the expression rate of a gene are located upstream (5′), within, and/or downstream (3′) relative to to the coding sequence of the nucleic acid molecule of the invention or another codogenic gene segment. They control in particular transcription and/or translation and/or the transcript stability. The expression level is dependent on the conjunction of further cellular regulatory systems, such as the protein biosynthesis and degradation systems of the cell.

Regulatory sequences include transcription and translation regulating sequences or signals, e.g. sequences located upstream (5′), which concern in particular the regulation of transcription or translation initiation, such as promoters or start codons, and sequences located downstream (3′), which concern in particular the regulation of transcription or translation termination and transcript stability, such as polyadenylation signals or stop codons. Regulatory sequences can also be present in transcribed coding regions as well in transcribed non-coding regions, e.g. in introns, as for example splicing sites, promoters for the regulation of expression of the nucleic acid molecule according to the invention in a cell and which can be employed are, in principle, all those which are capable of stimulating the transcription of genes in the organisms in question, such as microorganisms or plants. Suitable promoters, which are functional in these non-human organisms are generally known. They may take the form of constitutive or inducible promoters. Suitable promoters can enable the development- and/or tissue-specific expression in multi-cell eukaryotes; thus, leaf-, root-, flower-, seed-, stomata-, tuber-, fruit- or pollen-specific promoters may advantageously be used in plants. Furthermore in case of direct transformation of organelles such as plastids, promoters recognized by the plastid RNA-polymerases such as the plastid encoded Escherichia coli-like RNA polymerase or the nuclear encoded plastid RNA polymerase may advantageously be used.

The regulatory sequences or factors can, as described above, have a positive effect on, the expression of the genes introduced, thus increasing their expression. Thus, an enhancement of the expression can advantageously take place at the transcriptional level by using strong transcription signals such as strong promoters and/or strong enhancers. In addition, enhancement of expression on the translational level is also possible, for example by introducing translation enhancer sequences, e.g., the omega-enhancer e.g. improving the ribosomal binding to the transcript, or by increasing the stability of the mRNA, e.g. by replacing the 3′UTR coding region by a region encoding a 3′UTR known as conferring an high stability of the transcript or by stabilization of the transcript through the elimination of transcript instability, so that the mRNA molecule is translated more often than the wild type. For example in plants AU-rich elements (AREs) and DST (downstream) elements destabilized transcripts. Mutagenesis studies have demonstrated that residues within two of the conserved domains, the ATAGAT and the GTA regions, are necessary for instability function. Therefore removal or mutation of such elements would obviously lead to more stable transcripts, higher transcript rates and higher protein acitivity. Translation enhancers are also the “overdrive sequence”, which comprises the tobacco mosaic virus 5′-untranslated leader sequence and which increases the protein/RNA ratio (Gallie et al., Nucl. Acids Research 15, 8693 (1987)).

Enhancers are generally defined as cis active elements, which can stimulate gene transcription independent of position and orientation. Different enhancers have been identified in plants, which can either stimulate transcription constitutively or tissue or stimuli specific. Well known examples for constitutive enhancers are the enhancer from the 35S promoter (Odell et al., Nature 313, 810 (1985)) or the ocs enhancer (Fromm et al., Plant Cell 1, 977 (1989)). Other examples are the G-Box motif tetramer which confers high-level constitutive expression in dicotyledonous and monocotyledonous plants (Ishige et al., Plant Journal 18, 443 (1999)) or the petE, a NT-rich sequence which act as quantitative enhancers of gene expression in transgenic tobacco and potato plants (Sandhu et al., Plant Mol Biol. 37 (5), 885 (1998)). Beside that, a large variety of cis-active elements have been described which contribute to specific expression pattern, like organ specific expression or induced expression in response to biotic or abiotic stress. Examples are elements, which provide pathogen or wound-induced expression (Rushton, Plant Cell 14, 749 (2002)) or guard cell-specific expression (Plesch, Plant Journal 28, 455 (2001)).

Advantageous regulatory sequences for the expression of the nucleic acid molecule according to the invention in microorganisms are present for example in promoters such as the cos, tac, rha, trp, tet, trp-tet, Ipp, lac, !pp-lac, laclq-, T7, T5, T3, gal, trc, ara, SP6, λ-PR or λ-PL promoter, which are advantageously used in Gram-negative bacteria. Further advantageous regulatory sequences are present for example in the Gram-positive promoters amy, dnaK, xylS and SPO2, in the yeast or fungal promoters ADC1, MFα, AC, P-60, UASH, MCB, PHO, CYC1, GAPDH, TEF, rp28, ADH. Promoters, which are particularly advantageous, are constitutive, tissue or compartment specific or inducible promoters. In general, “promoter” is understood as meaning, in the present context, a regulatory sequence in a nucleic acid molecule, which mediates the expression of a coding sequence segment of a nucleic acid molecule. In general, the promoter is located upstream to the coding sequence segment. Some elements, for example expression-enhancing elements such as enhancer may, however, also be located downstream or even in the transcribed region.

In principle it is possible to use all natural promoters with their regulation sequences like those named above for the expression cassette according to the invention and the method according to the invention. Also, synthetic promoters may advantageously be used, either additionally or alone, for example synthetic promotors mediating seed-specific expression such as described in, for example, WO 99/16890. In the preparation of an expression cassette various DNA fragments can be manipulated in order to obtain a nucleotide sequence, which usefully reads in the correct direction and is equipped with a correct reading frame. To connect the DNA fragments (=nucleic acids according to the invention) to one another adaptors or linkers may be attached to the fragments. The promoter and the terminator regions can usefully be provided in the transcription direction with a linker or polylinker containing one or more restriction points for the insertion of this sequence. Generally, the linker has 1 to 10, mostly 1 to 8, preferably 2 to 6, restriction points. In general the size of the linker inside the regulatory region is less than 100 bp, frequently less than 60 bp, but at least 5 bp. The promoter may be both native or homologous as well as foreign or heterologous to the host non-human organism, for example to the host plant. In the 5′-3′ transcription direction the expression cassette contains the promoter, a nucleic acid molecule which is shown in the respective line in Table I, column 5 or 8, preferably the coding region, a homolog or a fragment thereof, and a region for transcription termination. Different termination regions can be exchanged for one another in any desired fashion.

As described above, the transcription of the genes introduced should advantageously be terminated by suitable terminators at the 3′ end of the genes introduced (behind the stop codon). A terminator, which may be used for this purpose is, for example, the OCS1 terminator, the nos3 terminator or the 35S terminator. As is the case with the promoters, different terminator sequences should be used for each gene in case several genes are introduced. Terminators, which are useful in microorganisms are for example the fimA terminator, txn terminator or trp terminator. Such terminators can be rho-dependent or rho-independent.

In an embodiment of the present invention the nucleic acid construct of the present invention comprises a nucleic acid molecule as depicted in the respective line in Table I, application no. 1, column 5 or 8, preferably the coding region thereof, a fragment or a homolog thereof, and a plant promoter, such as the Big 35S promoter, the DC3 promoter, the PC Ubi promoter, the LegB4-promoter, the Super promoter, the USP promoter or the like. In another embodiment the nucleic acid construct of the present invention comprises a nucleic acid molecule as depicted in th erespective line in Table I, application no. 1, column 5 or 8, preferably the coding region thereof, a fragment or a homolog thereof, and a plant promoter such as, for example, the USP, the LegB4-, the DC3 promoter or the ubiquitin promoter from parsley or other herein mentioned promoters. In a further embodiment the nucleic acid construct of the present invention comprises a nucleic acid molecule as depicted in th erespective line in Table I, application no. 1, column 5 or 8, preferably the coding region thereof, a fragment or a homolog thereof, and a plant promoter, such as the Big 35S promoter, the PC Ubi promoter, the Super promoter, the USP promoter or the like. In addition different terminators may advantageously be used in these nucleic acid constructs; examples for transcriptional termination are polyadenylation signals.

In order to ensure the stable integration, into the transgenic plant, of nucleic acid molecules used in the process according to the invention in combination with further genes, especially biosynthesis genes, over a plurality of generations, it may be advantageous to express each of the respective coding regions used in the process under the control of its own, preferably unique, promoter.

The nucleic acid construct is advantageously constructed in such a way that a promoter is followed by a suitable cleavage site for insertion of the nucleic acid to be expressed, advantageously in a polylinker, followed, if appropriate, by a terminator located behind the polylinker. If appropriate, this order is repeated several times so that several genes are combined in one construct and thus can be introduced into the transgenic plant in order to be expressed. The sequence is advantageously repeated up to three times. For the expression, the nucleic acid sequences are inserted via the above-mentioned suitable cleavage site, for example in the polylinker behind the promoter. It is advantageous for each nucleic acid sequence to have its own promoter and, if appropriate, its own terminator, as mentioned above. However, it is also possible to insert several nucleic acid sequences behind a promoter and, if appropriate, before a terminator if a polycistronic transcription is possible in the host or target cells. In this context, the insertion site, or the sequence of the nucleic acid molecules inserted, in the nucleic acid construct is not decisive, that is to say a nucleic acid molecule can be inserted in the first or last position in the cassette without this having a substantial effect on the expression. However, it is also possible to use only one promoter type in the construct.

Accordingly, in a preferred embodiment, the nucleic acid construct according to the invention confers expression of the nucleic acid molecule of the invention, and, optionally further genes, in a plant and comprises one or more plant regulatory elements. Said nucleic acid construct according to the invention advantageously encompasses beneath the nucleic acid molecule of the invention a plant promoter or a plant terminator or a plant promoter and a plant terminator. In another embodiment said nucleic acid construct according to the invention advantageously encompasses beneath the nucleic acid molecule of the invention a microorganism promoter or a microorganism terminator or a microorganism promoter and a microorganism terminator.

A “plant” promoter comprises regulatory elements, which mediate the expression of a coding sequence segment in plant cells. The plant promoter can originate from a plant cell, e.g. from the plant, which is transformed with the nucleic acid construct, expression cassette, or vector as described herein. However, a plant promoter does not need to be of plant origin, but may originate from viruses or microorganisms, in particular for example from viruses which attack plant cells. This also applies to other plant regulatory signals, for example in plant terminators. The term plant promoter shall also encompass organelle, especially plastidal promoters.

A nucleic acid construct suitable for plant expression preferably comprises regulatory elements which are capable of controlling the expression of genes in plant cells and which are operably linked so that each sequence can fulfill its function. Accordingly, the nucleic acid construct can also comprise transcription terminators. Examples for transcriptional termination are polyadenylation signals. Preferred polyadenylation signals are those which originate from Agrobacterium tumefaciens T-DNA, such as the gene 3 of the Ti plasmid pTiACH5, which is known as octopine synthase (Gielen et al., EMBO J. 3, 835 (1984)) or functional equivalents thereof, but all the other terminators which are functionally active in plants are also suitable.

Other preferred sequences for use in operable linkage in gene expression constructs are targeting sequences, which are required for targeting the gene product into specific cell compartments (for a review, see Kermode, Crit. Rev. Plant Sci. 15 (4), 285 (1996) and references cited therein), for example into the vacuole, the nucleus, all types of plastids, such as amyloplasts, chloroplasts, chromoplasts, the extracellular space, the mitochondria, the endoplasmic reticulum, elaioplasts, peroxisomes, glycosomes, and other compartments of cells or extracellular; preferred are sequences, which are involved in targeting to plastids as mentioned above. Sequences, which must be mentioned in this context are, in particular, the signal-peptide- or transit-peptide-encoding sequences which are known per se. For example, plastid-transit-peptide-encoding sequences enable the targeting of the expression product into the plastids of a plant cell. Targeting sequences are also known for eukaryotic and to a lower extent for prokaryotic organisms and can advantageously be operable linked with the nucleic acid molecule of the present invention as shown in Table I, application no. 1, columns 5 and 8, opreferably the coding region thereof, or homologs or fragments thereof, and described herein to achieve an expression in one of said compartments or extracellular.

For expression in plants, the nucleic acid molecule must, as described above, be linked operably to or comprise a suitable promoter which expresses the gene at the right point in time and in a cell- or tissue-specific manner. Usable promoters are constitutive promoters (Benfey et al., EMBO J. 8, 2195 (1989)), such as those which originate from plant viruses, such as 35S CAMV (Franck et al., Cell 21, 285 (1980)), 19S CaMV (see also U.S. Pat. No. 5,352,605 and WO 84/02913), 34S FMV (Sanger et al., Plant. Mol. Biol., 14, 433 (1990)), the parsley ubiquitin promoter, or plant promoters such as the Rubisco small subunit promoter described in U.S. Pat. No. 4,962,028 or the plant promoters PRP1 (Ward et al., Plant. Mol. Biol. 22 (1993)), SSU, PGEL1, OCS (Leisner, Proc. Natl. Acad. Sci. USA 85 (5), 2553 (1988)), lib4, usp, mas (Comai, Plant Mol. Biol. 15 (3), 373 (1990)), STLS1, ScBV (Schenk, Plant Mol. Biol. 39 (6),1221 (1999)), B33, SAD1 or SAD2 (flax promoters, Jain et al., Crop Science, 39 (6), 1696 (1999)) or nos (Shaw et al., Nucleic Acids Res. 12 (20), 7831 (1984)). Stable, constitutive expression of the proteins according to the invention into a plant can be advantageous. However, inducible expression of the polypeptide of the invention may be advantageous, if, for example, a late expression before the harvest is of advantage.

The expression of plant genes can also be facilitated as described above via a chemical inducible promoter (for a review, see Gatz, Annu. Rev. Plant Physiol. Plant Mol. Biol. 48, 89 (1997)). Chemically inducible promoters are particularly suitable when it is desired to express the gene in a time-specific manner. Examples of such promoters are a salicylic acid inducible promoter (WO 95/19443), a benzenesulfonamide inducible promoter (EP 388 186), and abscisic acid-inducible promoter (EP 335 528), a tetracyclin-inducible promoter (Gatz et al., Plant J. 2, 397 (1992)), a cyclohexanol- or ethanol-inducible promoter (WO 93/21334) or others as described herein.

Other suitable promoters are those which react to biotic or abiotic stress conditions, for example the pathogen-induced PRP1 gene promoter (Ward et al., Plant. Mol. Biol. 22, 361 (1993)), the auxin-induced GH3-promotor (Liu et al., Plant Cell 6, 645 (1994)), the cold/dehydration/ABA-induced Cor15A-promoter (Baker et al., Plant Mol. Biol. 24, 701 (1994)), the cold/drought/salt/ABA/wound-induced Cor78 promoter (Ishitani et al., Plant Cell 9, 1935 (1997), Yamaguchi-Shinozaki and Shinozaki, Plant Cell 6, 251 (1994)), the cold/dehydration-induced Rci2A-promoter (Capel et al., Plant Physiol 115, 569 (1997)), the drought/salt-induced Rd22-promoter (Yamaguchi-Shinozaki and Shinozaki, Mol. Gen. Genet. 238, 17 (1993)), the salt-induced RD29A-promoter (Yamaguchi-Shinozalei et al., Mol. Gen. Genet. 236, 331 (1993)), the salt-induced ARSK1-root promoter (Hwang and Goodman, Plant J. 8, 37 (1995), the salt-induced PtxA-root promoter (GenBank accession X67427), the tomato heat-inducible hsp80 promoter (U.S. Pat. No. 5,187,267), the potato chill-inducible alpha-amylase promoter (WO 96/12814) or the wound-inducible pinII promoter (EP-A-0 375 091) or others as described herein.

Preferred promoters are in particular those which bring about gene expression in tissues and organs, such as leaves, roots, seeds, pollen or xylem. Examples of tissue preferred or organ preferred promoters include, but are not limited to fruit-preferred, ovule-preferred, male tissue-preferred, seed-preferred, pollen-prefered integument-preferred, tuber-preferred, stalk-preferred, pericarp-preferred, leaf-preferred, stigma-preferred, pollen-preferred, anther-preferred, petal-preferred, sepal-preferred, pedicel-preferred, silique-preferred, stem-preferred, root-preferred promoters, and the like. Especially those promotors are preferred which bring about gene expression in tissues and organs in which the biosynthesis of amino acids takes place, like in seed cells, such as endosperm cells and cells of the developing embryo. Seed promoters are preferentially expressed during seed development and/or germination. For example, seed preferred promoters can be embryo-preferred, endosperm preferred, and seed coat-preferred (see Thompson et al., BioEssays 10, 108 (1989)). Examples of seed preferred promoters include, but are not limited to, cellulose synthase (celA), Cim1, gammazein, globulin-1, maize 19 kD zein (cZ19B1), and the like. Other suitable promoters are the oilseed rape napin gene promoter (U.S. Pat. No. 5,608,152), the Vicia faba USP promoter (Baeumlein et al., Mol Gen Genet, 225 (3), 459 (1991)), the Arabidopsis oleosin promoter (WO 98/45461), the Phaseolus vulgaris phaseolin promoter (U.S. Pat. No. 5,504,200), the Brassica Bce4 promoter (WO 91/13980), the bean arcs promoter, the carrot DcG3 promoter, or the Legumin B4 promoter (LeB4) (Baeumlein et al., Plant Journal, 2 (2), 233 (1992)), and promoters which bring about the seed-specific expression in monocotyledonous plants such as maize, barley, wheat, rye, rice and the like. Advantageous seed-specific promoters are the sucrose binding protein promoter (WO 00/26388), the phaseolin promoter and the napin promoter. Suitable promoters which must be considered are the barley Ipt2 or Ipt1 gene promoter (WO 95/15389 and WO 95/23230), and the promoters described in WO 99/16890 (promoters from the barley hordein gene, the rice glutelin gene, the rice oryzin gene, the rice prolamin gene, the wheat gliadin gene, the wheat glutelin gene, the maize zein gene, the oat glutelin gene, the sorghum kasirin gene and the rye secalin gene). Further suitable promoters are Amy32b, Amy 6-6 and Aleurain (U.S. Pat. No. 5,677,474), Bce4 (oilseed rape) (U.S. Pat. No. 5,530,149), glycinin (soya) (EP 571 741], phosphoenolpyruvate carboxylase (soya) (JP 06/62870), ADR12-2 (soya) (WO 98/08962), isocitrate lyase (oilseed rape) (U.S. Pat. No. 5,689,040])or α-amylase (barley) (EP 781 8499. Other promoters which are available for the expression of genes in plants are leaf-specific promoters such as those described in DE-A 19 644 478 or light-regulated promoters such as, for example, the pea petE promoter.

Further suitable plant promoters are the cytosolic FBPase promoter or the potato ST-LSI promoter (Stockhaus et al., EMBO J. 8, 2445 (1989)), the Glycine max phosphoribosylpyrophosphate amidotransferase promoter (GenBank Accession No. U87999) or the noduline-specific promoter described in EP 249 676.

Promoters, which are particularly suitable, are those which bring about plastid-specific expression. Suitable promoters such as the viral RNA polymerase promoter are described in WO 95/16783 and WO 97/06250, and the Arabidopsis clpP promoter, which is described in WO 99/46394.

Promoters, which are used for the strong expression of heterologous sequences as well as additional homologous sequences in as many tissues as possible, in particular also in leaves, are, in addition to several of the above-mentioned viral and bacterial promoters, preferably, plant promoters of actin or ubiquitin genes such as, for example, the rice actin1 promoter. Further examples of constitutive plant promoters are the sugarbeet V-ATPase promoters (WO 01/14572). Examples of synthetic constitutive promoters are the Super promoter (WO 95/14098) and promoters derived from G-boxes (WO 94/12015). If appropriate, chemical inducible promoters may furthermore also be used, as described in EP 388 186, EP 335 528, WO 97/06268.

Promoters, which are particularly suitable, are e.g. the super-promoter (Ni et al., Plant Journal 7, 661 (1995)), the ubiquitin promoter (Callis et al., J. Biol. Chem., 265, 12486 (1990); U.S. Pat. No. 5,510,474; U.S. Pat. No. 6,020,190; Kawalleck et al., Plant. Molecular Biology, 21, 673 (1993)) or the 34S promoter (GenBank Accession numbers M59930 and X16673). Promoters useful in the expression cassettes of the invention include, but are not limited to, the major chlorophyll a/b binding protein promoter, histone promoters, the Ap3 promoter, the β-conglycin promoter, the napin promoter, the soybean lectin promoter, the maize 15kD zein promoter, the 22kD zein promoter, the 27kD zein promoter, the g-zein promoter, the waxy, shrunken 1, shrunken 2 and bronze promoters, the Zm13 promoter (U.S. Pat. No. 5,086,169), the maize polygalacturonase promoters (PG) (U.S. Pat. Nos. 5,412,085 and 5,545,546), and the SGB6 promoter (U.S. Pat. No. 5,470,359), the rice cyclophilin promoter (Buchholz et al., Plant Mol. Biol.25 (5), 837 (1994)), the glutelin (rice) promoter (Takaiwa et al., Mol. Gen. Genet. 208, 15 (1986), Takaiwa et al., FEBS letts. 221, 43 (1987), the rice prolamin NRP33 promoter and the rice aglobulin Glb-1 promoter (Wu et al., Plant cell Physiology 39 (8), 885 (1998),as well as synthetic or other natural promoters.

As already mentioned herein, further regulatory sequences, which may be expedient, if appropriate, also include sequences, which target the transport and/or the localization of the expression products. Sequences, which must be mentioned in this context are, in particular, the signal-peptide- or transit-peptide-encoding sequences which are known per se. For example, plastid-transit-peptide-encoding sequences enable the targeting of the expression product into the plastids of a plant cell.

One embodiment of the present invention also relates to a method for generating a vector, which comprises the insertion of the nucleic acid molecule according to the invention or the expression cassette according to the invention into a nucleic acid molecule to give the vector. The vector can, for example, be introduced in to a cell, e.g. a microorganism or a plant cell, a plant or a part therof, as described herein for the nucleic acid construct, or below under transformation or transfection or shown in the examples. A transient or stable transformation of the host or target cell is possible, however, a stable transformation is preferred. The vector according to the invention is preferably a vector, which is suitable for expressing the polypeptide according to the invention in a microorganism or a plant cell, a plant or a part thereof.

The method can thus also encompass one or more steps for integrating regulatory signals into the vector, in particular signals, which mediate the expression in microorganisms or plant cells, plants or respective parts thereof.

Accordingly, the present invention also relates to a vector comprising the nucleic acid molecule characterized herein as part of a nucleic acid construct suitable for expression in plants and/or microrganisms or the nucleic acid molecule according to the invention.

The advantageous vectors of the invention comprise the nucleic acid molecules which encode proteins according to the invention, nucleic acid molecules which are used in the process, or nucleic acid construct suitable for plant expression or for microorganism expression comprising the nucleic acid molecules used, or expression cassettes according to the invention, either alone or in combination with further genes such as the biosynthesis or regulatory genes of the fine chemical metabolism e.g. with the genes mentioned herein above.

The recombinant expression vectors which are advantageously used in the process comprise the nucleic acid molecules according to the invention or the nucleic acid construct according to the invention, or ethe xpression cassettes according to the invention, in a form which is suitable for expressing, in a host cell. the nucleic acid molecules according to the invention or described herein. Accordingly, the recombinant expression vectors in addition may comprise one or more regulatory signals selected on the basis of the host cells to be used for the expression, in operable linkage with the nucleic acid sequence to be expressed. Furthermore, if desired, in addition the vector can comprise plastome sequences of the recipient organism to facilitate integration into the plastidal genome by homologous recombination as mentioned above.

The recombinant expression vectors used can be designed specifically for the expression, in prokaryotic and/or eukaryotic cells, of nucleic acid molecules used in the process. This is advantageous since intermediate steps of the vector construction are frequently carried out in microorganisms for the sake of simplicity. For example, the genes according to the invention and other genes can be expressed in bacterial cells, insect cells (using baculovirus expression vectors), yeast cells and other fungal cells (Romanos, Yeast 8, 423 (1992); van den Hondel, in “More Gene Manipulations in Fungi”, ed. Bennet J. W., Lasure L. L., pp. 396-428, Academic Press, San Diego (1991); van den Hondel C. A. M. J. J., in “Applied Molecular Genetics of Fungi”, ed. Peberdy J. F. et al., pp. 1-28, Cambridge University Press, Cambridge(1991)), algae (Falciatore et al., Marine Biotechnology.1 (3), 239 (1999)) using vectors and following a transformation method as described in WO 98/01572, and preferably in cells of multi-celled plants (see Schmidt R. and Willmitzer L., Plant Cell Rep. 7, 583 (1988); “Plant Molecular Biology and Biotechnology”, C Press, Boca Raton, Fla., chapter 6/7, pp.71-119 (1993); White F. F., in “Transgenic Plants”, Bd. 1, Engineering and Utilization, ed.Kung and Wu R., Academic Press, 128-43 (1993); Potrykus, Annu. Rev. Plant Physiol. Plant Molec. Biol. 42, 205 (1991) (and references cited therein)). Suitable host cells are furthermore discussed in Goeddel, “Gene Expression Technology: Methods in Enzymology 185”, Academic Press, San Diego, Cailf. (1990). As an alternative, the sequence of the recombinant expression vector can be transcribed and translated in vitro, for example using T7 promotor-regulatory sequences and T7 polymerase.

In the event it is necessary proteins can be expressed in prokaryotes using vectors comprising constitutive or inducible promoters, which control the expression of fusion proteins or nonfusion proteins as mentioned above.

Other vectors which are suitable in prokaryotic organisms are known to the skilled worker; these vectors are for example in E. coli pLG338, pACYC184, the pBR series, such as pBR322, the pUC series such as pUC18 or pUC19, the M113mp series, pKC30, pRep4, pHS1, pHS2, pPLc236, pMBL24, pLG200, pUR290, pIN-111113-B1, lambda-gt11 or pBdCl, in Streptomyces pIJ101, pIJ364, pIJ702 or pIJ361, in Bacillus pUB110, pC194 or pBD214, in Corynebacterium pSA77 or pAJ667.

In a further embodiment, the expression vector is a yeast expression vector. Examples of vectors for expression in the yeasts S. cerevisiae encompass pYeDesaturasec1 (Baldari et al., Embo J. 6, 229 (1987)), pMFa (Kurjan and Herskowitz, Cell 30, 933 (1982)), pJRY88 (Schultz et al., Gene 54, 113(1987)) and pYES2 (Invitrogen Corporation, San Diego, Calif.). Vectors and methods for the construction of vectors which are suitable for use in other fungi, such as the filamentous fungi, encompass those which are described in detail in: van den Hondel C. A. M. J. J. in “Applied Molecular Genetics of Fungi”, Peberdy J. F., ed., pp. 1-28, Cambridge University Press, Cambridge (1991); or van den Hondel C. A. M. J. J. in “More Gene Manipulations in Fungi”, Bennet J. W.& Lasure L. L., ed., pp. 396-428, Academic Press, San Diego (1991). Examples of other suitable yeast vectors are 2alphaM, pAG-1, YEp6, YEp13 or pEMBLYe23.

Further vectors, which may be mentioned by way of example, are pALS1, μl L2 or pBB116 in fungi or pLGV23, pGHlac+, pBIN19, pAK2004 or pDH51 in plants.

As an alternative, the nucleic acid sequences can be expressed in insect cells using baculovirus expression vectors. Baculovirus vectors, which are available for expressing proteins in cultured insect cells (for example Sf9 cells) encompass the pAc series (Smith et al., Mol. Cell Biol. 3, 2156 (1983)) and the pVL series (Lucklow and Summers, Virology 170, 31 (1989)).

The above-mentioned vectors are only a small overview of potentially suitable vectors. Further plasmids are known to the skilled worker and are described, for example, in “Cloning Vectors” (ed. Pouwels P.H., et al., Elsevier, Amsterdam-New York-Oxford, 1985, ISBN 0 444 904018). Further suitable expression systems for prokaryotic and eukaryotic cells, see the chapters 16 and 17 by Sambrook J., Fritsch E. F. and Maniatis T., “Molecular Cloning: A Laboratory Manual”, 2^{nd} edition, Cold Spring Harbor Laboratory, Cold Spring Harbor Laboratory Press, Cold Spring Harbor, N. Y., 1989.

An embodiment of the invention relates to a vector where the nucleic acid molecule according to the invention is linked operably to regulatory sequences which permit the expression in a prokaryotic or eukaryotic host. A further embodiment of the invention relates to a vector in which a nucleic acid sequence encoding one of the polypeptides shown in therespective line in Table II, application no. 1, columns 5 or 8, or homologs or fragments thereof, is functionally linked to a targeting sequence such as a plastidial or mitochondrial targeting sequence. A further embodiment of the invention relates to a vector in which a nucleic acid sequence encoding one of the polypeptides shown in the respective line in Table II, application no. 1, columns 5 or 8, or homologs or fragments thereof, is functionally linked to (a) regulatory sequence which permit the expression in an organelle,such as plastids or mitochondria, preferably plastids.

A further embodiment of the invention relates to a vector which comprises a nucleic acid sequence encoding one of the polypeptides shown in the respective line in Table II, application no. 1, columns 5 or 8, or homologs or fragments thereof, but no additional targeting sequence.

Accordingly, one embodiment of the invention relates to a host cell, which has been transformed stably or transiently with the vector according to the invention or the nucleic acid molecule according to the invention or the nucleic acid construct according to the invention or the expression construct according to the invention.

The genetically modified cells may be regenerated to the respective non-human organism, for example a plant cell to a plant, by all of the methods known to those skilled in the art. Appropriate methods can be found in the publications referred to above by Kung S. D. and Wu R., Potrykus or Hofgen and Willmitzer.

Accordingly, a further aspect of the invention relates to transgenic non-human organisms transformed by at least one nucleic acid molecule, expression cassette or vector according to the invention as well as cells, cell cultures, tissue, parts—such as, for example, leaves, roots, pollen, etc. in the case of plant organisms—or reproductive material derived from such non-human organisms. The terms “host organism”, “host cell”, “recombinant (host) non-human organism” and “transgenic (host) cell” are used here interchangeably. Of course these terms relate not only to the particular host non-human organism or the particular target cell but also to the descendants or potential descendants of these non-human organisms or cells. Since, due to mutation or environmental effects certain modifications may arise in successive generations, these descendants need not necessarily be identical with the parental cell but nevertheless are still encompassed by the term as used here.

For the purposes of the invention “ transgenic” or “recombinant” means with regard for example to a nucleic acid molecule, a nucleic acid construct, an expression cassette or a vector containing the nucleic acid molecule according to the invention or an non-human organism transformed with/by the nucleic acid molecule, nucleic acid construct, expression cassette or vector according to the invention all those constructions produced by genetic engineering methods in which either
(a) the nucleic acid molecule depicted in the respective line in Table I, column 5 or 8, preferably the coding region thereof, or homologs or fragments thereof; or
(b) a genetic control sequence functionally linked to the nucleic acid molecule described under (a), for example a 3′- and/or 5′- genetic control sequence such as a promoter or terminator, or
(c) (a) and (b);
   are not found in their natural, genetic environment or have been modified by genetic engineering methods, wherein the modification may by way of example be a substitution, addition, deletion, inversion or insertion of one or more nucleotide residues. Natural genetic environment means the natural genomic or chromosomal locus in the organism of origin or inside the host non-human organism or presence in a genomic library. In the case of a genomic library the natural genetic environment of the nucleic acid sequence is preferably retained at least in part. The environment sequence borders the nucleic acid sequence at least on one side and has a sequence length of at least 50 bp, preferably at least 500 bp, particularly preferably at least 1,000 bp, most particularly preferably at least 5,000 bp. A naturally occurring expression cassette—for example the naturally occurring combination of the natural promoter of the nucleic acid sequence according to the invention with the corresponding gene—turns into a transgenic expression cassette when the latter is modified by unnatural, synthetic (“artificial”) methods such as by way of example a mutagenesis. In case a non-human organism is transformed by a homologous nucleic acid molecule or by a homologous expression cassette the resulting non-human organism represents also a respective transgenic non-human organism since the additional homologous nucleic acid molecule or the additional homologous expression cassette is not located in its original envirnment. Appropriate methods are described by way of example in U.S. Pat. No. 5,565,350 or WO 00/15815. In another embodiment the non-human organism comprises a heterologous nucleic acid molecule.

Suitable organisms or host organisms for the nucleic acid molecule, nucleic acid construct, expression cassette or vector according to the invention are advantageously in principle all non-human organisms, which are suitable for the expression of recombinant genes as described above. Preferred non-human organism are microorganism or plants as described above, in particular those plants, which can be transformed in a suitable manner. These include monocotyledonous and dicotyledonous plants. In an embodiment of the present invention plants like Arabidopsis, Asteraceae such as Calendula or crop plants such as soybean, peanut, castor oil plant, sunflower, flax, corn, cotton, oilseed rape, coconut, oil palm, safflower (Carthamus tinctorius) or cocoa bean are preferred. In another embodiment agriculturally useful plants such as cereals and grasses, for example Triticum spp., Zea mays, Hordeum vulgare, oats, Secale cereale, Oryza sativa, Pennisetum glaucum, Sorghum bicolor, Triticale, Agrostis spp., Cenchrus ciliaris, Dactylis glomerata, Festuca arundinacea, Lolium spp., Medicago spp. and Saccharum spp., legumes and oil crops, for example Brassica juncea, Brassica napus, Glycine max, Arachis hypogaea, Gossypium hirsutum, Cicer arietinum, Helianthus annuus, Lens culinaris, Linum usitatissimum, Sinapis alba, Trifolium repens and Vicia narbonensis, vegetables and fruits, for example bananas, grapes, Lycopersicon esculentum, asparagus, cabbage, watermelons, kiwi fruit, Solanum tuberosum, Beta vulgaris, cassava and chicory, trees, for example Coffea species, Citrus spp., Eucalyptus spp., Picea spp., Pinus spp. and Populus spp., medicinal plants and trees, and flowers are preferred.

In another embodiment of the invention host plants for the nucleic acid molecule, nucleic acid construct, expression cassette or vector according to the invention are selected from the group comprising corn, soy, oil seed rape (including canola and winter oil seed rape), cotton, wheat and rice.

A further object of the invention relates to the use of a nucleic acid construct, e.g. an expression cassette, comprising one or more nucleic acid molecules encoding one or more polypeptides as depicted in the respective line(s) in Table II, application no. 1, columns 5 or 8, or homologs or fragments thereof, or comprising one or more nucleic acid molecules as depicted in the respective line(s) in Table I, application no. 1, columns 5 or 8, preferably the coding region thereof, or homologs or fragments thereof, or comprising a nucleic acid molecule hybridizing therewith for the transformation of a microorganism or a plant cell, plant tissues or a part of a plant

In doing so, depending on the choice of promoter, the nucleic acid molecules shown in the respective line in Table I, application no. 1, column 5 or 8, preferably the coding region thereof, or homologs or fragments thereof, or the nucleic acid molecules encoding a polypeptide as depicted in the respective line in Table II, application no. 1, column 5 or 8, or homologs or fragments thereof, can be expressed in the whole plant or specifically e.g. in the leaves, in the seeds, the nodules, in roots, in the stem or other parts of the plant. Those transgenic plants overproducing sequences, e.g. as depicted in the respective line in Table I, application no. 1, column 5 or 8, preferably the coding region thereof, or homologs or fragments thereof, the plant cell, plant tissues, plants, the reproductive material thereof, or parts thereof are a further object of the present invention.

Within the framework of the present invention, the production of the methionine is due to the generation or over-expression of one or more polypeptides as depicted in the respective line(s) in Table II, application no. 1, column 5 or 8, or homologs or fragments thereof, or encoded by the corresponding nucleic acid molecules as depicted in the respective line(s) in Table I, application no. 1, column 5 or 8, preferably the coding region thereof, or homologs or fragments thereof, in the non-human organism according to the invention, advantageously in the transgenic microorganism or plant according to the invention, at least for the duration of at least one plant generation.

A constitutive expression of the polypeptide as depicted in the respective line in Table II, application no. 1, column 5 or 8, or homologs or fragments thereof, or encoded by the respective nucleic acid molecule as depicted in the respective line in Table I, application no. 1, column 5 or 8, preferably the coding region thereof, or homologs or fragments thereof, may be advantageous. On the other hand, an inducible expression may also appear desirable. Expression of the polypeptide sequences of the invention can be either directed to the cytosol or to the organelles, such as plastids or mitochondria, preferably the plastids of the host cells, preferably the plant cells, or non-targeted.

The efficiency of the expression of the polypeptides depicted in the respective line in Table II, colunm 5 or 8, or homologs or fragments thereof, or encoded by the respective nucleic acid molecule as depicted in the respective line in Table I, column 5 or 8, preferably the coding region thereof, or homologs or fragments thereof, can be determined, for example, on test plants in greenhouse trials.

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 1, column 5 or 8, preferably shown in Table II A, application no. 1, column 5, or in Table IIA, application no. 1, column 8, or in Table II B, application no. 1, column 8, or a homolog or a fragment thereof;
   (b) a nucleic acid molecule shown in Table I, application no. 1, column 5 or 8, preferably shown in Table I A, application no. 1, column 5, or in Table I A, application no. 1, column 8, or in Table I B, application no. 1, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 1, preferably shown in Table II A, application no. 1, column 5, or in Table II A, application no. 1, column 8, or in Table II B, application no. 1, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 1, preferably shown in Table I A, application no. 1, column 5, or in Table I A, application no. 1, column 8,or in Table I B, application no. 1, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridisation conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 1;
   (i) a nucleic acid molecule encoding a polypeptide having the activity represented by a respective protein as depicted in column 5 of Table II, application no. 1;
   (j) a nucleic acid molecule comprising a polynucleotide which is obtained by amplifying a cDNA library or a genomic library using the primers in Table III, application no. 1, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
      or a nucleic acid molecule comprising a sequence which is complementary thereto; and, if desired
2) nucleic acid molecule encoding a transit peptide, preferably encoding a plastidal transit peptide or a mitochondrial transit peptide; which are operable linked;]
   or comprising an expression cassette comprising
[0) a promoter, prefereably a promoter selected from the group consisting of Big35S, PCUbi, Super and USP; and
1) a nucleic acid molecule selected from the group consisting of
   (a) a nucleic acid molecule encoding a polypeptide shown in Table II, application no. 1, column 5 or 8, preferably shown in Table II A, application no. 1, column 5, or in Table II A, application no. 1, column 8, or in Table II B, application no. 1, column 8, or a homolog or a fragment thereof;
   (b) a nucleic acid molecule shown in Table I, application no. 1, column 5 or 8, preferably shown in Table I A, application no. 1, column 5, or in Table I A, application no. 1, column 8, or in Table I B, application no. 1, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 1, preferably shown in Table II A, application no. 1, column 5, or in Table II A, application no. 1, column 8, or in Table II B, application no. 1, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 1, preferably shown in Table I A, application no. 1, column 5, or in Table I A, application no. 1, column 8,or in Table I B, application no. 1, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridisation conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 1;
   (i) a nucleic acid molecule encoding a polypeptide having the activity represented by a respective protein as depicted in column 5 of Table II, application no. 1;
   (j) a nucleic acid molecule comprising a polynucleotide which is obtained by amplifying a cDNA library or a genomic library using the primers in Table III, application no. 1, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide; which are operable linked;]
   or comprising or being transformed by a vector comprising
[1) a nucleic acid molecule selected from the group consisting of
   (a) a nucleic acid molecule encoding a polypeptide shown in Table II, application no. 1, column 5 or 8, preferably shown in Table II A, application no. 1, column 5, or in Table II A, application no. 1, column 8, or in Table II B, application no. 1, column 8, or a homolog or a fragment thereof;
   (b) a nucleic acid molecule shown in Table I, application no. 1, column 5 or 8, preferably shown in Table I A, application no. 1, column 5, or in Table I A, application no. 1, column 8, or in Table I B, application no. 1, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 1, preferably shown in Table II A, application no. 1, column 5, or in Table II A, application no. 1, column 8, or in Table II B, application no. 1, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 1, preferably shown in Table I A, application no. 1, column 5, or in Table I A, application no. 1, column 8,or in Table I B, application no. 1, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridisation conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 1;
   (i) a nucleic acid molecule encoding a polypeptide having the activity represented by a respective protein as depicted in column 5 of Table II, application no. 1;
   (j) a nucleic acid molecule comprising a polynucleotide which is obtained by amplifying a cDNA library or a genomic library using the primers in Table III, application no. 1, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 1.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 1 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 1, in the same line as of the respective nucleic acid molecule according to 1) “plastidic” is depicted, or encoding a mitochondrial transit peptide, especially in case in column 6 of Table I, application no. 1, in the same line as of the respective nucleic acid molecule according to 1) “mitochondrial” is depicted.

In another embodiment of said transgenic non-human organism the expression cassette does not comprise 2) a nucleic acid molecule encoding a transit peptide, especially in case in column 6 of Table I, application no. 1, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 1.

In another preferred embodiment of said transgenic non-human organism said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 1, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 1, in the same line as of the respective nucleic acid molecule according to 1) “plastidic” is depicted, or encoding a mitochondrial transit peptide, especially in case in column 6 of Table I, application no. 1, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 1, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 1, in the same line as of the respective nucleic acid molecule according to 1) “plastidic” is depicted, or encoding a mitochondrial transit peptide, especially in case in column 6 of Table I, application no. 1, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, especially in case in column 6 of Table I, application no. 1, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 1, in the same line as of the respective nucleic acid molecule according to 1) “plastidic” is depicted, or encoding a mitochondrial transit peptide, especially in case in column 6 of Table I, application no. 1, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 1.

In another embodiment of said transgenic non-human organism said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi,

Super and USP; a nucleic acid molecule encoding a transit peptide, especially in case in column 6 of Table I, application no. 1, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 1.

In another embodiment of said transgenic non-human organism said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 1, in the same line as of the respective nucleic acid molecule according to 1) “plastidic” is depicted, or encoding a mitochondrial transit peptide, especially in case in column 6 of Table I, application no. 1, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, especially in case in column 6 of Table I, application no. 1, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 1, in the same line as of the respective nucleic acid molecule according to 1) “plastidic” is depicted, or encoding a mitochondrial transit peptide, especially in case in column 6 of Table I, application no. 1, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 1 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, especially in case in column 6 of Table I, application no. 1, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 1 (in the same line) and confers a generation or an increase of the respective fine chemical in a non-human organism or a part thereof as compared to a corresponding wild-type non-human organism or part thereof.

In a prefered embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression casssette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 1, column 5 or 8, preferably shown in Table II A, application no. 1, column 5, or in Table IIA, application no. 1, column 8, or in Table II B, application no. 1, column 8, or a homolog or a fragment thereof;
   (b) a nucleic acid molecule shown in Table I, application no. 1, column 5 or 8, preferably shown in Table I A, application no. 1, column 5, or in Table I A, application no. 1, column 8, or in Table I B, application no. 1, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 1, preferably shown in Table II A, application no. 1, column 5, or in Table II A, application no. 1, column 8, or in Table II B, application no. 1, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 1, preferably shown in Table I A, application no. 1, column 5, or in Table I A, application no. 1, column 8,or in Table I B, application no. 1, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridisation conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 1;
   (i) a nucleic acid molecule encoding a polypeptide having the activity represented by a respective protein as depicted in column 5 of Table II, application no. 1;
   (j) a nucleic acid molecule comprising a polynucleotide which is obtained by amplifying a cDNA library or a genomic library using the primers in Table III, application no. 1, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising an expression cassette comprising
[0) a promoter, prefereably a promoter selected from the group consisting of Big35S, PCUbi, Super and USP; and
1) a nucleic acid molecule selected from the group consisting of
   (a) a nucleic acid molecule encoding a polypeptide shown in Table II, application no. 1, column 5 or 8, preferably shown in Table II A, application no. 1, column 5, or in Table II A, application no. 1, column 8, or in Table II B, application no. 1, column 8, or a homolog or a fragment thereof;
   (b) a nucleic acid molecule shown in Table I, application no. 1, column 5 or 8, preferably shown in Table I A, application no. 1, column 5, or in Table I A, application no. 1, column 8, or in Table I B, application no. 1, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 1, preferably shown in Table II A, application no. 1, column 5, or in Table II A, application no. 1, column 8, or in Table II B, application no. 1, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 1, preferably shown in Table I A, application no. 1, column 5, or in Table I A, application no. 1, column 8,or in Table I B, application no. 1, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridisation conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 1;
   (i) a nucleic acid molecule encoding a polypeptide having the activity represented by a respective protein as depicted in column 5 of Table II, application no. 1;
   (j) a nucleic acid molecule comprising a polynucleotide which is obtained by amplifying a cDNA library or a genomic library using the primers in Table III, application no. 1, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      and, if desired
2) nucleic acid molecule encoding a transit peptide, preferably encoding a plastidal transit peptide or a mitochondrial transit peptide;
   which are operable linked;]
   or comprising or being transformed by a vector comprising
[1) a nucleic acid molecule selected from the group consisting of
   (a) a nucleic acid molecule encoding a polypeptide shown in Table II, application no. 1, column 5 or 8, preferably shown in Table II A, application no. 1, column 5, or in Table II A, application no. 1, column 8, or in Table II B, application no. 1, column 8, or a homolog or a fragment thereof;
   (b) a nucleic acid molecule shown in Table I, application no. 1, column 5 or 8, preferably shown in Table I A, application no. 1, column 5, or in Table I A, application no. 1, column 8, or in Table I B, application no. 1, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 1, preferably shown in Table II A, application no. 1, column 5, or in Table II A, application no. 1, column 8, or in Table II B, application no. 1, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 1, preferably shown in Table I A, application no. 1, column 5, or in Table I A, application no. 1, column 8,or in Table I B, application no. 1, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridisation conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 1;
   (i) a nucleic acid molecule encoding a polypeptide having the activity represented by a respective protein as depicted in column 5 of Table II, application no. 1;
   (j) a nucleic acid molecule comprising a polynucleotide which is obtained by amplifying a cDNA library or a genomic library using the primers in Table III, application no. 1, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      whereby, preferably, the nucleic acid molecule according to 1 (a), (b), (c), (d), (e), (f), (g), (h), (i), (j) or (k) distinguishes over the sequence depicted in Table I A and/or I B, application no. 1, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 1, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 1, column 5 or 8, or the coding region thereof, respectively. In a further embodiment the nucleic acid molecule does not encode the polypeptide sequence shown in Table II A and/or II B, application no. 1, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 1, column 5 or 8 and does not encode a protein of the sequence shown in Table II A and/or II B, application no. 1, column 5 or 8. Accordingly, in one embodiment, the protein encoded by a sequence of a nucleic acid according to 1 (a), (b), (c), (d), (e), (f), (g), (h), (i), (j) or (k) does not consist of the sequence shown in Table I A and/or I B, application no. 1, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 1, column 5 or 8 but less than 100%, preferably less than 99.999%, 99.99%, 99.9%, 99%, 98%, 97%, 96% or 95% identical to the sequence shown in Table II A and/or II B, application no. 1, column 5 or 8;
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 1.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 1 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 1, in the same line as of the respective nucleic acid molecule according to 1) “plastidic” is depicted, or encoding a mitochondrial transit peptide, especially in case in column 6 of Table I, application no. 1, in the same line as of the respective nucleic acid molecule according to 1) “mitochondrial” is depicted.

In another embodiment thereof the expression cassette does not comprise 2) a nucleic acid molecule encoding a transit peptide, especially in case in column 6 of Table I, application no. 1, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 1.

In another preferred embodiment of said transgenic non-human organism said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the same line in column 5 of Table II, application no. 1, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 1, in the same line as of the respective nucleic acid molecule according to 1) “plastidic” is depicted, or encoding a mitochondrial transit peptide, especially in case in column 6 of Table I, application no. 1, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 1, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 1, in the same line as of the respective nucleic acid molecule according to 1) “plastidic” is depicted, or encoding a mitochondrial transit peptide, especially in case in column 6 of Table I, application no. 1, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, especially in case in column 6 of Table I, application no. 1, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 1, in the same line as of the respective nucleic acid molecule according to 1) “plastidic” is depicted, or encoding a mitochondrial transit peptide, especially in case in column 6 of Table I, application no. 1, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 1.

In another embodiment of said transgenic non-human organism said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, especially in case in column 6 of Table I, application no. 1, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 1.

In another embodiment of said transgenic non-human organism said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 1, in the same line as of the respective nucleic acid molecule according to 1) “plastidic” is depicted, or encoding a mitochondrial transit peptide, especially in case in column 6 of Table I, application no. 1, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, especially in case in column 6 of Table I, application no. 1, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 1, in the same line as of the respective nucleic acid molecule according to 1) “plastidic” is depicted, or encoding a mitochondrial transit peptide, especially in case in column 6 of Table I, application no. 1, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 1 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, especially in case in column 6 of Table I, application no. 1, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 1 and confers a generation or an increase of the respective fine chemical in a non-human organism or a part thereof as compared to a corresponding wild-type non-human organism or part thereof.

In a prefered embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression casssette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

In an embodiment thereof the transgenic non-human organism is a microorganism selected from the group consisting of Charophyceae such as the genera Chara, Nitella e.g. the species Chara globularis, Chara vulgaris, Nitella flexilis, Chlorophyceae such as the genera Acrosiphonia, Spongomorpha, Urospora, Bryopsis, Pseudobryopsis, Trichosolen, Dichotomosiphon, Caulerpa, Rhipilia, Blastophysa, Avrainvillea, Chlorodesmis, Codium, Espera, Halicystis, Halimeda, Penicillus, Pseudocodium, Rhipiliopsis, Rhipocephalus, Tydemania, Udotea, Derbesia, Acrochaete, Aphanochaete, Bolbocoleon, Chaetobolus, Chaetonema, Chaetophora, Chlorotylium, Desmococcus, Draparnaldia, Draparnaldiopsis, Ectochaete, Endophyton, Entocladia, Epicladia, Internoretia, Microthamnion, Ochlochaete, Phaeophila, Pilinella, Pringsheimiella, Protoderma, Pseudendoclonium, Pseudodictyon, Pseudopringsheimia, Pseudulvella, Schizomeris, Stigeoclonium, Thamniochaete, Ulvella, Pilinia, Tellamia, Helicodictyon, Actidesmium, Ankyra, Characium, Codiolum, Sykidion, Keratococcus, Prototheca, Bracteacoccus Chlorococcum, Excentrosphaera, Hormidium, Oophila, Schroederia, Tetraedron, Trebouxia Chlorosarcinopsis, Gomphonitzschia, Coccomyxa, Dactylothece, Diógenes, Disporá, Gloeocystis, Mycanthococcus, Ourococcus, Coelastrum, Dicranochaete, Botryococcus, Dictyosphaerium, Dimorphococcus, Chlorochytrium, Kentrosphaera, Phyllobium, Gomontia, Hormotila, Euastropsis, Hydrodictyon, Pectodictyon, Pediastrum, Sorastrum, Tetrapedia, Acanthosphaera, Echinosphaerella, Echinosphaeridium, Errerella, Gloeoactinium, Golenkeniopsis Golenkinia, Micractinium, Ankistrodesmus, Chlorella, Chodatella, Closteriopsis, Cryocystis, Dactylococcus, Dematractum, Eremosphaera, Eutetramorus, Franceia, Glaucocystis, Gloeotaenium, Kirchneriella, Lagerheimiella, Monoraphidium, Nannochloris, Nephrochlamys, Nephrocytium, Oocystis, Oonephris, Pachycladon, Palmellococcus, Planktosphaeria, Polyedriopsis, Pseudoraciborskia, Quadrigula, Radiococcus, Rochiscia, Scotiella, Selanastrum, Thorakochloris, Treubaria, Trochiscia, Westella, Zoochlorella, Ostreobium, Phyllosiphon, Protosiphon, Rhodochytrium, Actinastrum, Coronastrum, Crucigenia, Dictymocystis, Enallax, Scenedesmus, Selenastrum, Tetradesmus, Tetrallantos, Tetrastrum, Chlorosarcina, Anadyomene, Valoniopsis, Ventricaria, Basicladia, Chaetomorpha, Cladophora, LolaPithophoraRhizoclonium Chaetosphaeridium, Conochaete, Coleochaete, Oligochaetophora, Polychaetophora, Cylindrocapsa, Gongrosira, Protococcus, Acetabularia, Batophora, Bornetella, Dasycladus, Halicoryne, Neomeris, Elakatothrix, Raphidonema, Microspora, Bulbochaete, Oedocladium, Oedogonium, Prasiola, Rosenvingiella, Schizogonium, Apjohnia, Chamaedoris, Cladophoropsis, Siphonocladus, Spongocladia, Boergesenia, Boodlea, Cystodictyon, Dictyosphaeria, Ernodesmis, Microdictyon, Struvea, Valonia, Sphaeroplea, Malleochloris, Stylosphaeridium, Gloeococcus, Palmella, Palmodictyon, Palmophyllum, Pseudospherocystis, Sphaerocystis, Urococcus, Apiocystis, Chaetopeltis, Gemellicystis, Paulschulzia, Phacomyxa, Pseudotetraspora, Schizochlamys, Tetraspora, Cephaleuros, Ctenocladus, Epibolium, Leptosira, Trentepohlia, Diplochaete, Monostroma, Binuclearia, Geminella, Klebsormidium, Planetonema, Radiofilum, Stichococcus, Ulothrix, Uronema, Blidingia, Capsosiphon, Chloropelta, Enteromorpha, Percursaria, Ulva, UIvaria, Brachiomonas, Carteria, Chlainomonas, Chlamydomonas, Chlamydonephris, Chlorangium, Chlorogonium, Cyanidium, Fortiella, Glenomonas, Gloeomonas, Hyalogonium, Lobomonas Polytoma, Pyramichlamys, Scourfieldia, Smithsonimonas, Sphaerellopsis, Sphenochloris, Spirogonium, Collodictyon, Dunaliella, Haematococcus, Stephanosphaera, Coccomonas, Dysmorphococcus, Phacotus, Pteromonas, Thoracomonas, Wislouchiella, Mascherina, Pyrobotrys, Spondylomorum, Eudorina, Gonium, Oltmannsiella, Pandorina, Platydorina, Pleodorina, Stephanoon, Volvox, Volvulina, Actinotaenium, Arthrodesmus, Bambusina Closterium, Cosmarium, Desmidium, Euastrum, Groenbladia, Hyalotheca, Micrasterias, Penium, Phymatodocis, Pleurotaenium, Sphaerozosma, Spinoclosterium, Spinocosmarium, Spondylosium, Staurastrum, Tetmemorus, Triploceras, Xanthidium, Cylindrocystis, Genicularia, Gonatozygon, Mesotaenium, Netrium, Roya, Spirotaenia, Cosmocladium, Debarya, Docidium, Euastridium, Hallasia, Mougeotia, Mougeotiopsis, Sirogonium, Spirogyra, Staurodesmus, Teilingia, Zygnema, Zygogonium, e.g. the species Caulerpa taxifolia, Prototheca wickerhamii, Ankistrodesmus falcatus, Chlorella ellipsoidea, Chlorella pyrenoidosa, Clorella sorokiniana, Chlorella vulgaris, Scenedesmus obliquus, Scenedesmus quadricauda, Selenastrum capricornutum, Selenastrum undecimnotata, Cladophora glomerata, Chlamydomonas eugametos, Chlamydomonas reinhardtii, Cyanidium caldarium, Dunaliella salina, Dunaliella tertiolecta, Euglena gracilis, Haematococcus pluvialis, Coniugatophyceae, Prasinophyceae Trebouxiophyceae, Ulvophyceae, Chlorodendraceae, Pedinomonadales, Halosphaeraceae, Pterospermataceae, Monomastigaceae, Pyramimonadaceae, Chlorodendraceae such as the genera Prasinocladus e.g. the species Prasinocladus ascus, Halosphaeraceae, Pedinomonadales, Pedinomonadaceae such as the genera Pedinomonas, Pterospermataceae such as the genera Pachysphaera, Pterosperma, Halosphaera, Pyramimonas, Bacillariophyceae, Chrysophyceae, Craspedophyceae, Euglenophyceae, Prymnesiophyceae, Phaeophyceae, Dinophyceae, Rhodophyceae, Xanthophyceae, Prasinophyceae such as the generaNephroselmis, Prasinococcus, Scherffelia, Tetraselmis, Mantoniella, Ostreococcus e.g. the species Nephroselmis olivacea, Prasinococcus capsulatus, Scherffelia dubia, Tetraselmis chui, Tetraselmis suecica, Mantoniella squamata or Ostreococcus tauri.

In an embodiment thereof the transgenic non-human organism is a plant (or a part thereof), preferably a monocotyledonous or a dicotyledonous plant. Preferably the plant is selected from the group consisting of Anacardiaceae such as the genera Pistacia, Mangifera, Anacardium e.g. the species Pistacia vera [pistachios, Pistazie], Mangifer indica [Mango] or Anacardium occidentale [Cashew]; Asteraceae such as the genera Calendula, Carthamus, Centaurea, Cichorium, Cynara, Helianthus, Lactuca, Locusta, Tagetes, Valeriana e.g. the species Calendula officinalis [Marigold], Carthamus tinctorius [safflower], Centaurea cyanus [cornflower], Cichorium intybus [blue daisy], Cynara scolymus [Artichoke], Helianthus annus [sunflower], Lactuca sativa, Lactuca crispa, Lactuca esculenta, Lactuca scariola L. ssp. sativa, Lactuca scariola L. var. integrate, Lactuca scariola L. var. integrifolia, Lactuca sativa subsp. romana, Locusta communis, Valeriana locusta [lettuce], Tagetes lucida, Tagetes erecta or Tagetes tenuifolia [Marigold]; Apiaceae such as the genera Daucus e.g. the species Daucus carota [carrot]; Betulaceae such as the genera Corylus e.g. the species Corylus avellana or Corylus colurna [hazelnut]; Boraginaceae such as the genera Borago e.g. the species Borago officinalis [borage]; Brassicaceae such as the genera Brassica, Melanosinapis, Sinapis, Arabadopsis e.g. the species Brassica napus, Brassica rapa ssp. [canola, oilseed rape, turnip rape], Sinapis arvensis Brassica juncea, Brassica juncea var. juncea, Brassica juncea var. crispifolia, Brassica juncea var. foliosa, Brassica nigra, Brassica sinapioides, Melanosinapis communis [mustard], Brassica oleracea [fodder beet] or Arabidopsis thaliana; Bromeliaceae such as the genera Anana, Bromelia e.g. the species Anana comosus, Ananas ananas or Bromelia comosa [pineapple]; Caricaceae such as the genera Carica e.g. the species Carica papaya [papaya]; Cannabaceae such as the genera Cannabis e.g. the species Cannabis sative [hemp], Convolvulaceae such as the genera Ipomea, Convolvulus e.g. the species Ipomoea batatus, Ipomoea pandurata, Convolvulus batatas, Convolvulus tiliaceus, Ipomoea fastigiata, Ipomoea tiliacea, Ipomoea triloba or Convolvulus panduratus [sweet potato, Man of the Earth, wild potato], Chenopodiaceae such as the genera Beta, i.e. the species Beta vulgaris, Beta vulgaris var. altissima, Beta vulgaris var. Vulgaris, Beta maritima, Beta vulgaris var. perennis, Beta vulgaris var. conditiva or Beta vulgaris var. esculenta [sugar beet]; Cucurbitaceae such as the genera Cucubita e.g. the species Cucurbita maxima, Cucurbita mixta, Cucurbita pepo or Cucurbita moschata [pumpkin, squash]; Elaeagnaceae such as the genera Elaeagnus e.g. the species Olea europaea [olive]; Ericaceae such as the genera Kalmia e.g. the species Kalmia latifolia, Kalmia angustifolia, Kalmia microphylla, Kalmia polifolia, Kalmia occidentalis, Cistus chamaerhodendros or Kalmia lucida [American laurel, broad-leafed laurel, calico bush, spoon wood, sheep laurel, alpine laurel, bog laurel, western bog-laurel, swamp-laurel]; Euphorbiaceae such as the genera Manihot, Janipha, Jatropha, Ricinus e.g. the species Manihot utilissima, Janipha manihot, Jatropha manihot, Manihot aipil, Manihot dulcis, Manihot manihot, Manihot melanobasis, Manihot esculenta [manihot, arrowroot, tapioca, cassava] or Ricinus communis [castor bean, Castor Oil Bush, Castor Oil Plant, Palma Christi, Wonder Tree]; Fabaceae such as the genera Pisum, Albizia, Cathormion, Feuillea, Inga, Pithecolobium, Acacia, Mimosa, Medicajo, Glycine, Dolichos, Phaseolus, Soja e.g. the species Pisum sativum, Pisum arvense, Pisum humile [pea], Albizia berteriana, Albizia julibrissin, Albizia lebbeck, Acacia berteriana, Acacia littoralis, Albizia berteriana, Albizzia berteriana, Cathormion berteriana, Feuillea berteriana, Inga fragrans, Pithecellobium berterianum, Pithecellobium fragrans, Pithecolobium berterianum, Pseudalbizzia berteriana, Acacia julibrissin, Acacia nemu, Albizia nemu, Feuilleea julibrissin, Mimosa julibrissin, Mimosa speciosa, Sericanrda julibrissin, Acacia lebbeck, Acacia macrophylla, Albizia lebbek, Feuilleea lebbeck, Mimosa lebbeck, Mimosa speciosa [bastard logwood, silk tree, East Indian Walnut], Medicago sativa, Medicago falcata, Medicago varia [alfalfa], Glycine max [soybean], Dolichos soja, Glycine gracilis, Glycine hispida, Phaseolus max or Soja hispida; Geraniaceae such as the genera Pelargonium, Cocos, Oleum e.g. the species Cocos nucifera, Pelargonium grossularioides or Oleum cocois [coconut]; Gramineae such as the genera Saccharum e.g. the species Saccharum officinarum; Juglandaceae such as the genera Juglans, Wallia e.g. the species Juglans regia, Juglans ailanthifolia, Juglans sieboldiana, Juglans cinerea, Wallia cinerea, Juglans bixbyi, Juglans californica, Juglans hindsii, Juglans intermedia, Juglans jamaicensis, Juglans major, Juglans microcarpa, Juglans nigra or Wallia nigra [walnut, black walnut, common walnut, persian walnut, white walnut, butternut, black walnut]; Lauraceae such as the genera Persea, Laurus e.g. the species laurel Laurus nobilis [bay, laurel, bay laurel, sweet bay], Persea americana Persea americana, Persea gratissima or Persea persea [avocado]; Leguminosae such as the genera Arachis e.g. the species Arachis hypogaea [peanut]; Linaceae such as the genera Linum, Adenolinum e.g. the species Linum usitatissimum, Linum humile, Linum austriacum, Linum bienne, Linum angustifolium, Linum catharticum, Linum flavum, Linum grandiflorum, Adenolinum grandiflorum, Linum lewisii, Linum narbonense, Linum perenne, Linum perenne var. lewisii, Linum pratense or Linum trigynum [flax, linseed]; Lythrarieae such as the genera Punica e.g. the species Punica granatum [pomegranate]; Malvaceae such as the genera Gossypium e.g. the species Gossypium hirsutum, Gossypium arboreum, Gossypium barbadense, Gossypium herbaceum or Gossypium thurberi [cotton]; Musaceae such as the genera Musa e.g. the species Musa nana, Musa acuminata, Musa paradisiaca, Musa spp. [banana]; Onagraceae such as the genera Camissonia, Oenothera e.g. the species Oenothera biennis or Camissonia brevipes [primrose, evening primrose]; Palmae such as the genera Elacis e.g. the species Elaeis guineensis [oil plam]; Papaveraceae such as the genera Papaver e.g. the species Papaver orientale, Papaver rhoeas, Papaver dubium [poppy, oriental poppy, corn poppy, field poppy, shirley poppies, field poppy, long-headed poppy, long-pod poppy]; Pedaliaceae such as the genera Sesamum e.g. the species Sesamum indicum [sesame]; Piperaceae such as the genera Piper, Artanthe, Peperomia, Steffensia e.g. the species Piper aduncum, Piper amalago, Piper angustifolium, Piper auritum, Piper betel, Piper cubeba, Piper longum, Piper nigrum, Piper retrofractum, Artanthe adunca, Artanthe elongata, Peperomia elongata, Piper elongatum, Steffensia elongata. [Cayenne pepper, wild pepper]; Poaceae such as the genera Hordeum, Secale, Avena, Sorghum, Andropogon, Holcus, Panicum, Oryza, Zea, Triticum e.g. the species Hordeum vulgare, Hordeum jubatum, Hordeum murinum, Hordeum secalinum, Hordeum distichon Hordeum aegiceras, Hordeum hexastichon, Hordeum hexastichum, Hordeum irregulare, Hordeum sativum, Hordeum secalinum [barley, pearl barley, foxtail barley, wall barley, meadow barley], Secale cereale [rye], Avena sativa, Avena fatua, Avena byzantina, Avena fatua var. sativa, Avena hybrida [oat], Sorghum bicolor, Sorghum halepense, Sorghum saccharatum, Sorghum vulgare, Andropogon drummondii, Holcus bicolor, Holcus sorghum, Sorghum aethiopicum, Sorghum arundinaceum, Sorghum caffrorum, Sorghum cernuum, Sorghum dochna, Sorghum drummondii, Sorghum durra, Sorghum guineense, Sorghum lanceolatum, Sorghum nervosum, Sorghum saccharatum, Sorghum subglabrescens, Sorghum verticilliflorum, Sorghum vulgare, Holcus halepensis, Sorghum miliaceum millet, Panicum militaceum [Sorghum, millet], Oryza sativa, Oryza latifolia [rice], Zea mays [corn, maize] Triticum aestivum, Triticum durum, Triticum turgidum, Triticum hybernum, Triticum macha, Triticum sativum or Triticum vulgare [wheat, bread wheat, common wheat], Proteaceae such as the genera Macadamia e.g. the species Macadamia intergrifolia [macadamia]; Rubiaceae such as the genera Coffea e.g. the species Cofea spp., Coffea arabica, Coffea canephora or Coffea liberica [coffee]; Scrophulariaceae such as the genera Verbascum e.g. the species Verbascum blattaria, Verbascum chaixii, Verbascum densiflorum, Verbascum lagurus, Verbascum longifolium, Verbascum lychnitis, Verbascum nigrum, Verbascum olympicum, Verbascum phlomoides, Verbascum phoenicum, Verbascum pulverulentum or Verbascum thapsus [mullein, white moth mullein, nettle-leaved mullein, dense-flowered mullein, silver mullein, long-leaved mullein, white mullein, dark mullein, greek mullein, orange mullein, purple mullein, hoary mullein, great mullein]; Solanaceae such as the genera Capsicum, Nicotiana, Solanum, Lycopersicon e.g. the species Capsicum annuum, Capsicum annuum var. glabriusculum, Capsicum frutescens [pepper], Capsicum annuum [paprika], Nicotiana tabacum, Nicotiana alata, Nicotiana attenuata, Nicotiana glauca, Nicotiana langsdorffii, Nicotiana obtusifolia, Nicotiana quadrivalvis, Nicotiana repanda, Nicotiana rustica, Nicotiana sylvestris [tobacco], Solanum tuberosum [potato], Solanum melongena [egg-plant] (Lycopersicon esculentum, Lycopersicon lycopersicum, Lycopersicon pyriforme, Solanum integrifolium or Solanum lycopersicum [tomato]; Sterculiaceae such as the genera Theobroma e.g. the species Theobroma cacao [cacao]; Theaceae such as the genera Camellia e.g. the species Camellia sinensis) [tea]

Particular preference is given to transgenic crop plants such as by way of example barley, wheat, rye, oats, corn, soybean, rice, cotton, sugar beet, oilseed rape and canola, sunflower, flax, hemp, thistle, potatoes, tobacco, tomatoes, tapioca, cassava, arrowroot, alfalfa, lettuce and the various tree, nut and vine species; especial preference is given to monocotyledonous crop plants like corn, wheat or rice; in another embodiment special preference is given to dicotyledonous crop plants like soy bean, oil seed rape (including canola and winter oil seed rape), cotton.

The term “transgenic plants” used in accordance with the invention also refers to the progeny of a transgenic plant, for example the T1, T2, T3 and subsequent plant generations or the BC1, BC2, BC3 and subsequent plant generations. Thus, the transgenic plants according to the invention can be raised and selfed or crossed with other individuals in order to obtain further transgenic plants according to the invention. Transgenic plants may also be obtained by propagating transgenic plant cells vegetatively. The present invention also relates to transgenic plant material, which can be derived from a transgenic plant population according to the invention. Such material includes plant cells and certain tissues, organs and parts of plants in all their manifestations, such as seeds, leaves, anthers, fibers, tubers, roots, root hairs, stems, embryo, calli, cotelydons, petioles, harvested material, plant tissue, reproductive tissue, pollen, and cell cultures, which are derived from the actual transgenic plant and/or can be used for bringing about the transgenic plant. Any transformed plant obtained according to the invention can be used in a conventional breeding scheme or in in vitro plant propagation to produce more transformed plants with the same characteristics and/or can be used to introduce the same characteristic in other varieties of the same or related species. Such plants are also part of the invention. Seeds obtained from the transformed plants genetically also contain the same characteristic and are part of the invention. As mentioned before, the present invention is in principle applicable to any plant and crop that can be transformed with any of the transformation method known to those skilled in the art.

An “isolated” nucleic acid molecule is one that is substantially separated from other nucleic acid molecules, which are present in the natural source of the nucleic acid. That means other nucleic acid molecules are present in an amount less than 5% based on weight of the amount of the desired nucleic acid, preferably less than 2% by weight, more preferably less than 1% by weight, most preferably less than 0.5% by weight. Preferably, an “isolated” nucleic acid is free of some of the sequences that naturally flank the nucleic acid (i.e., sequences located at the 5′ and 3′ ends of the nucleic acid) in the genomic DNA of the non-human organism from which the nucleic acid is derived. For example, in various embodiments, the FCRP encoding nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be free from some of the other cellular material with which it is naturally associated, or culture medium when produced by recombinant techniques, or chemical precursors or other chemicals when chemically synthesized.

A nucleic acid molecule of the present invention, e.g., a nucleic acid molecule encoding a FCRP, especially the coding region thereof, or a portion thereof which confers the production or increased production of the fine chemical, can be isolated using standard molecular biological techniques and the sequence information provided herein. For example, an A. thaliana FCRP encoding cDNA can be isolated from a A. thaliana c-DNA library or a E. coli, Saccharomyces cerevisiae, Synechocystis sp., Brassica napus, Glycine max, Zea mays or Oryza sativa FCRP encoding cDNA can be isolated from a E. coli, Saccharomyces cerevisiae, Synechocystis sp., Brassica napus, Glycine max, Zea mays or Oryza sativa c-DNA library respectively using all or portion of one of the respective sequences. Moreover, a nucleic acid molecule encompassing all or a portion of one of the sequences of Table I can be isolated by the polymerase chain reaction using oligonucleotide primers designed based upon this sequence. For example, mRNA can be isolated from plant cells (e.g., by the guanidinium-thiocyanate extraction procedure of Chirgwin et al., Biochemistry 18, 5294 (1979)) and cDNA can be prepared using reverse transcriptase (e.g., Moloney MLV reverse transcriptase, available from Gibco/BRL, Bethesda, MD; or AMV reverse transcriptase, available from Seikagaku America, Inc., St. Petersburg, Fla.). Synthetic oligonucleotide primers for polymerase chain reaction amplification can be designed based upon one of the nucleotide sequences shown in Table I. A nucleic acid molecule of the invention can be amplified using cDNA or, alternatively, genomic DNA, as a template and appropriate oligonucleotide primers according to standard PCR amplification techniques. The nucleic acid molecule so amplified can be cloned into an appropriate vector and characterized by DNA sequence analysis. Furthermore, oligonucleotides corresponding to a FCRP encoding nucleotide sequence can be prepared by standard synthetic techniques, e.g., using an automated DNA synthesizer.

In an embodiment, an isolated nucleic acid molecule of the invention comprises one of the nucleic acid molecules as shown in Table I, especially the coding region thereof, and if desired, as well as a 5′ untranslated sequence and 3′ untranslated sequence.

Moreover, the nucleic acid molecule of the invention can comprise only a portion of the coding region of one of the nucleic acid molecule of Table I, for example, a fragment which can be used as a probe or primer or a fragment encoding a biologically active portion of a FCRP.

Portions of proteins encoded by the FCRP encoding nucleic acid molecules of the invention are preferably biologically active portions described herein. As used herein, the term “biologically active portion of” a FCRP is intended to include a portion, e.g. a domain/motif, of the protein being responsible for the ability of the protein to enable the production or increased production of the fine chemical. To determine whether a FCRP, or a biologically active portion thereof, results in a production or an increased production of the fine chemical in the non-human organism, like a microorganism or a plant, an analysis of the non-human organism comprising the FCRP may be performed. Such analysis methods are well known to those skilled in the art, as detailed in the Examples. More specifically, nucleic acid fragments encoding biologically active portions of a FCRP can be prepared by isolating a portion of one of the sequences of the nucleic acid of Table I, e.g. expressing the encoded portion of the FCRP or peptide (e.g. by recombinant expression in vitro) and assessing the activity of the encoded portion of the FCRP or peptide.

Biologically active portions of a FCRP are encompassed by the present invention and include peptides comprising amino acid sequences derived from the amino acid sequence of a FCRP encoding gene, or the amino acid sequence of a protein homologous to a FCRP, which include fewer amino acids than a full length FCRP or the full length protein which is homologous to a FCRP, and exhibits at least some enzymatic or biological activity of a FCRP.

Typically, biologically active portions (e.g., peptides which are, for example, 5, 10, 15, 20, 30, 35, 36, 37, 38, 39, 40, 50, 75, 100, 125, 150 or more amino acids in length) comprise a domain or motif with at least one activity of a FCRP. Moreover, other biologically active portions in which other regions of the protein are deleted, can be prepared by recombinant techniques and evaluated for one or more of the activities described herein. Preferably, the biologically active portions of a FCRP include one or more selected domains/motifs or portions thereof having biological activity.

The term “biological active portion” or “biological activity” means a polypeptide as depicted in the respective line in Table II, application no. 1, column 3 or a portion of said polypeptide which still has at least 10% or 20%, preferably 30%, 40%, 50% or 60%, especially preferably 70%, 75%, 80%, 90% or 95% of the enzymatic or biological activity of the natural or starting enzyme or protein.

In the process according to the invention nucleic acid sequences or molecules can be used, which, if appropriate, contain synthetic, non-natural or modified nucleotide bases, which can be incorporated into DNA or RNA. Said synthetic, non-natural or modified bases can for example increase the stability of the nucleic acid molecule outside or inside a cell. The nucleic acid molecules of the invention can contain the same modifications as aforementioned.

As used in the present context the term “nucleic acid molecule” may also encompass the untranslated sequence or molecule located at the 3′ and at the 5′ end of the coding gene region, for example at least 500, preferably 200, especially preferably 100, nucleotides of the sequence upstream of the 5′ end of the coding region and at least 100, preferably 50, especially preferably 20, nucleotides of the sequence downstream of the 3′ end of the coding gene region. It is often advantageous only to choose the coding region for cloning and expression purposes.

Preferably, the nucleic acid molecule used in the process according to the invention or the nucleic acid molecule of the invention is an isolated nucleic acid molecule. In one embodiment, the nucleic acid molecule of the invention is the nucleic acid molecule used in the process of the invention.

The nucleic acid molecules used in the process, for example the polynucleotide of the invention or of a part thereof can be isolated using molecular-biological standard techniques and the sequence information provided herein. Also, for example a homologous sequence or homologous, conserved sequence regions at the DNA or amino acid level can be identified with the aid of comparison algorithms. The former can be used as hybridization probes under standard hybridization techniques (for example those described in Sambrook et al., “Molecular Cloning: A Laboratory Manual” 2nd ed., Cold Spring Harbor Laboratory, Cold Spring Harbor Laboratory Press, Cold Spring Harbor, N.Y., 1989) for isolating further nucleic acid sequences useful in this process.

Synthetic oligonucleotide primers for the amplification, e.g. as shown in the respective line in Table III, application no. 1, column 8, by means of polymerase chain reaction can be generated on the basis of a sequence shown herein, for example the sequence shown in the respective line in Table I, application no. 1, columns 5 or 8, or the sequences derived from Table II, application no. 1, columns 5 or 8, respectively.

Moreover, it is possible to identify a conserved region of a protein from various organism by carrying out protein sequence alignments with the polypeptide encoded by the nucleic acid molecules of the present invention, in particular with the sequences encoded by the nucleic acid molecule shown in the respective line in column 5 or 8 of Table I, application no. 1, from which conserved regions, and in turn, degenerate primers can be derived. Conserved regions are those, which show a very little variation in the amino acid in one particular position of several homologs from different origin. The consensus sequence and polypeptide motifs shown in the respective line in column 8 of Table IV, application no. 1, are derived from such alignments of homologous proteins.

In an embodiment of the present invention, in the method of the present invention the activity of a polypeptide comprising or consisting of a consensus sequence or at least one polypeptide motif shown in the respective line in Table IV, application no.1, column 8, is increased. In another embodiment thereof, the method of the present invention relates to a polypeptide comprising or consisting of a consensus sequence shown in the respective line in Table IV, application no. 1, column 8 or comprising at least one polypeptide motif shown in the respective line in Table IV, application no. 1, column 8, whereby not more than 20, 15, 10, 9, 8, 7, 6, 5 4, 3, 2 or 1, or 0 of the amino acid positions indicated can be replaced by any amino acid. In another embodiment thereof, the method of the present invention relates to a polypeptide comprising or consisting of a consensus sequence shown in the respective line in Table IV, application no. 1, column 8 or comprising at least one polypeptide motif shown in the respective line in Table IV, application no. 1, column 8, whereby not more than 15%, 10%, 5%, 4%, 3%, 2% or 1%, or 0% of the amino acid positions defined as distinct specific amino acids are/is replaced by another amino acid. In another embodiment thereof, the method of the present invention relates to a polypeptide comprising or consisting of a consensus sequence shown in the respective line in Table IV, application no. 1, column 8 or comprising at least one polypeptide motif shown in the respective line in Table IV, application no. 1, column 8, whereby not more than than 20, 15, 10, 9, 8, 7, 6, 5, 4, 3, 2 or 1, or 0 amino acids are inserted into a consensus sequence or polypeptide motif.

In an embodiment of the present invention, in the method of the present invention the activity of a polypeptide comprising or consisting of a consensus sequence or at least one polypeptide motif shown in the respective line in Table IV, application no.1, column 8, is increased, whereby said polypeptide distinguishes over the sequence depicted in Table II, application no. 1, columns 5 or 8 by one or more amino acids.

In one embodiment, said polypeptide of the invention distinguishes over the sequence shown in the respective line in Table II, application no. 1, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II B, application no. 1, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in Table II, application no. 1, columns 5 or 8.

The consensus sequence was derived from a multiple alignment of the sequences as listed in a single line of Table II. The amino acids are given in their three letter code. In case a specific amino acid is indicated this specific amino acid is conserved in at least 80% of the aligned proteins whereas the three letter code XAA stands for amino acids, which are not conserved in at least 80% of the aligned sequences. The consensus sequence starts with the first conserved amino acid in the alignment, and ends with the last conserved amino acid in the alignment of the investigated sequences.

Patterns had to match at least 80% of the investigated proteins. Conserved patterns were identified with the software tool MEME version 3.5.1 or manually by using first standard prosite anotation. The number of given x indicates the distances between conserved amino acid residues (given in the one letter code), e.g. (1) the pattern Y-x(21,23)-F means that conserved tyrosine and phenylalanine residues in the alignment are separated from each other by minimum 21 and maximum 23 amino acid residues in the alignment of all investigated sequences, (2) the pattern Y-x(21,23)-[FW] means that a conserved tyrosine is separated by minimum 21 and maximum 23 amino acid residues from either a phenylalanine or tryptophane. However, these patterns have been “translated” into the WIPO standard 25. MEME was developed by Bailey Timothy L.and Elkan Charles, Dept. of Computer Science and Engeneering, University of California, San Diego, USA and is described by Bailey Timothy L. and Elkan Charles (Proceedings of the Second International Conference on Intelligent Systems for Molecular Biology, pp. 28-36, AAAI Press, Menlo Park, Calif., 1994). The source code for the stand-alone program is public available from the San Diego Supercomputer centre (http://meme.sdsc.edu). For identifying common motifs in all sequences with the software tool MEME, the following settings were used: -maxsize 500000, -nmotifs 15, -evt 0.001, -maxw 60, -distance 1e-3, - minsites number of sequences used for the analysis. Input sequences for MEME were non-aligned sequences in Fasta format. Other parameters were used in the default settings in this software version. Prosite patterns for conserved domains were generated with the software tool Pratt version 2.1 or manually. Pratt was developed by Jonassen Inge, Dept. of Informatics, University of Bergen, Norway and is described by Jonassen et al. (Jonassen I., Collins J. F. and Higgins D. G., Protein Science 4, 1587 (1995); Jonassen I., Efficient discovery of conserved patterns using a pattern graph, Submitted to CABIOS Febr. 1997). The source code (ANSI C) for the stand-alone program is public available, e.g. at establisched Bioinformatic centers like EBI (European Bioinformatics Institute). For generating patterns with the software tool Pratt, following settings were used: PL (max Pattern Length): 100, PN (max Nr of Pattern Symbols): 100, PX (max Nr of consecutive x′s): 30, FN (max Nr of flexible spacers): 5, FL (max Flexibility): 30, FP (max Flex.Product): 10, ON (max number patterns): 50. Input sequences for Pratt were distinct regions of the protein sequences exhibiting high similarity as identified from software tool MEME. The minimum number of sequences, which have to match the generated patterns (CM, min Nr of Seqs to Match) was set to at least 80% of the provided sequences. Parameters not mentioned here were used in their default settings. The Prosite patterns of the conserved domains can be used to search for protein sequences matching this pattern. Various established Bioinformatic centres provide public internet portals for using those patterns in database searches (e.g. PIR (Protein Information Resource, located at Georgetown University Medical Center) or ExPASy (Expert Protein Analysis System)). Alternatively, stand-alone software is available, like the program Fuzzpro, which is part of the EMBOSS software package. For example, the program Fuzzpro not only allows to search for an exact pattern-protein match but also allows to set various ambiguities in the performed search.

The alignment was performed with the software ClustalW (version 1.83) and is described by Thompson et al. (Nucleic Acids Research 22, 4673 (1994)). The source code for the stand-alone program is public available from the European Molecular Biology Laboratory; Heidelberg, Germany. The analysis was performed using the default parameters of ClustalW v1.83 (gap open penalty: 10.0; gap extension penalty: 0.2; protein matrix: Gonnet; protein/DNA endgap: -1; protein/DNA gapdist: 4).

Degenerated primers can then be utilized by PCR for the amplification of fragments of novel proteins having above-mentioned activity, e.g. conferring the production or the increased production of the fine chemical as compared to a corresponding, non-transformed, wild type cell, or non-human organism, like a plant cell or a plant or a part thereof, after generating or increasing the expression or activity or having the activity of a protein as shown in therespective line in Table II, column 3 or further functional homologs of the polypeptide of the invention from other non-human organisms.

These fragments can then be utilized as hybridization probe for isolating the complete gene sequence. As an alternative, the missing 5′ and 3′ sequences can be isolated by means of RACE-PCR. A nucleic acid molecule according to the invention can be amplified using cDNA or, as an alternative, genomic DNA as template and suitable oligonucleotide primers, following standard PCR amplification techniques. The nucleic acid molecule amplified thus can be cloned into a suitable vector and characterized by means of DNA sequence analysis. Oligonucleotides, which correspond to one of the nucleic acid molecules used in the process can be generated by standard synthesis methods, for example using an automatic DNA synthesizer.

Nucleic acid molecules which are advantageously for the process according to the invention can be isolated based on their homology to the nucleic acid molecules disclosed herein using the sequences or part thereof as or for the generation of a hybridization probe and following standard hybridization techniques under stringent hybridization conditions. In this context, it is possible to use, for example, isolated one or more nucleic acid molecules of at least 15, 20, 25, 30, 35, 40, 50, 60 or more nucleotides, preferably of at least 15, 20 or 25 nucleotides in length which hybridize under stringent conditions with the above-described nucleic acid molecules, in particular with those which encompass a nucleotide sequence of the nucleic acid molecule used in the process of the invention or encoding a protein used in the invention or of the nucleic acid molecule of the invention. Nucleic acid molecules with 30, 50, 100, 250 or more nucleotides may also be used.

The term “homology” means that the respective nucleic acid molecules or the encoded proteins are functionally and/or structurally equivalent. The nucleic acid molecules that are homologous to the nucleic acid molecules described above and that are derivatives of said nucleic acid molecules are, for example, variations of said nucleic acid molecules which represent modifications having the same biological function, in particular encoding proteins with the same or substantially the same biological function. They may be naturally occurring variations, such as sequences from other plant varieties or species, or mutations. These mutations may occur naturally or may be obtained by mutagenesis techniques. The allelic variations may be naturally occurring allelic variants as well as synthetically produced or genetically engineered variants. Structurally equivalents can, for example, be identified by testing the binding of said polypeptide to antibodies or computer based predictions. Structurally equivalents have the similar immunological characteristic, e.g. comprise similar epitopes.

By “hybridizing” it is meant that such nucleic acid molecules hybridize under conventional hybridization conditions, preferably under stringent conditions such as described by, e.g., Sambrook (“Molecular Cloning; A Laboratory Manual”, 2^{nd} edition, Cold Spring Harbor Laboratory Press, Cold Spring Harbor, N.Y. (1989)) or in “Current Protocols in Molecular Biology”, John Wiley & Sons, N.Y. (1989), chapter 6.3.1-6.3.6.)

According to the invention, DNA as well as RNA molecules of the corresponding nucleic acid molecules of the invention can be used as probes. Further, as template for the identification of functional homologues Northern blot assays as well as Southern blot assays can be performed. The Northern blot assay advantageously provides further information about the expressed gene product: e.g. expression pattern, occurrence of processing steps, like splicing and capping, etc. The Southern blot assay provides additional information about the chromosomal localization and organization of the gene being related to the nucleic acid molecule of the invention.

A preferred, non-limiting example of stringent hybridization conditions are hybridizations in 6× sodium chloride/sodium citrate (=SSC) at approximately 45° C., followed by one or more wash steps in 0.2×SSC, 0.1% SDS at 50 to 65° C., for example at 50° C., 55° C. or 60° C. The skilled worker knows that these hybridization conditions differ as a function of the type of the nucleic acid and, for example when organic solvents are present, with regard to the temperature and concentration of the buffer. The temperature under “standard hybridization conditions” differs for example as a function of the type of the nucleic acid between 42° C. and 65° C., preferably between 45° C. and 50° C. in an aqueous buffer with a concentration of 0.1×, 0.5×, 1×, 2×, 3×, 4× or 5×SSC (pH 7.2). If organic solvent(s) is/are present in the above-mentioned buffer, for example 50% formamide, the temperature under standard conditions is approximately 40° C., 42° C. or 45° C. The hybridization conditions for DNA:DNA hybrids are preferably for example 0.1×SSC and 20° C., 25° C., 30° C., 35° C., 40° C. or 45° C., preferably between 30° C. and 45° C. The hybridization conditions for DNA:RNA hybrids are preferably for example 0.1×SSC and 30° C., 35° C., 40° C., 45° C., 50° C. or 55° C., preferably between 45° C. and 55° C. The above-mentioned hybridization temperatures are determined for example for a nucleic acid approximately 100 by (=base pairs) in length and a G +C content of 50% in the absence of formamide. The skilled worker knows to determine the hybridization conditions required with the aid of textbooks, for example the ones mentioned above, or from the following textbooks: Sambrook et al., “Molecular Cloning”, Cold Spring Harbor Laboratory, 1989; Hames and Higgins (ed.), “Nucleic Acids Hybridization: A Practical Approach”, IRL Press at Oxford University Press, Oxford, 1985; Brown (ed.), “Essential Molecular Biology: A Practical Approach”, IRL Press at Oxford University Press, Oxford, 1991.

A further example of one such stringent hybridization condition is hybridization at 4×SSC at 65° C., followed by a washing in 0.1×SSC at 65° C. for one hour. Alternatively, an exemplary stringent hybridization condition is in 50% formamide, 4×SSC at 42° C. Further, the conditions during the wash step can be selected from the range of conditions delimited by low-stringency conditions (approximately 2×SSC at 50° C.) and high-stringency conditions (approximately 0.2×SSC at 50° C., preferably at 65° C.) (20×SSC:0.3 M sodium citrate, 3 M NaCl, pH 7.0). In addition, the temperature during the wash step can be raised from low-stringency conditions at room temperature, approximately 22° C., to higher-stringency conditions at approximately 65° C. Both of the parameters salt concentration and temperature can be varied simultaneously, or else one of the two parameters can be kept constant while only the other is varied. Denaturants, for example formamide or SDS, may also be employed during the hybridization. In the presence of 50% formamide, hybridization is preferably effected at 42° C. Relevant factors like 1) length of treatment, 2) salt conditions, 3) detergent conditions, 4) competitor DNAs, 5) temperature and 6) probe selection can be combined case by case so that not all possibilities can be mentioned herein.

Thus, in a preferred embodiment, Northern blots are prehybridized with RothiHybri-Quick buffer (Roth, Karlsruhe) at 68° C. for 2 h. Hybridization with radioactive labelled probe is done overnight at 68° C. Subsequent washing steps are performed at 68° C. with 1×SSC. For Southern blot assays the membrane is prehybridized with Rothi-Hybri-Quick buffer (Roth, Karlsruhe) at 68° C. for 2 h. The hybridzation with radioactive labelled probe is conducted over night at 68° C. Subsequently the hybridization buffer is discarded and the filter shortly washed using 2×SSC; 0.1% SDS. After discarding the washing buffer new 2×SSC; 0,1% SDS buffer is added and incubated at 68° C. for 15 minutes. This washing step is performed twice followed by an additional washing step using 1×SSC; 0.1% SDS at 68° C. for 10 min.

Some examples of conditions for DNA hybridization (Southern blot assays) and wash step are shown herein below:

(1) Hybridization conditions can be selected, for example, from the following conditions:
(a) 4×SSC at 65° C.,
(b) 6×SSC at 45° C.,
(c) 6×SSC, 100 mg/ml denatured fragmented fish sperm DNA at 68° C.,
(d) 6×SSC, 0.5% SDS, 100 mg/ml denatured salmon sperm DNA at 68° C.,
(e) 6×SSC, 0.5% SDS, 100 mg/ml denatured fragmented salmon sperm DNA, 50% formamide at 42° C.,
(f) 50% formamide, 4×SSC at 42° C.,
(g) 50% (v/v) formamide, 0.1% bovine serum albumin, 0.1% Ficoll, 0.1% polyvinylpyrrolidone, 50 mM sodium phosphate buffer pH 6.5, 750 mM NaCl, 75 mM sodium citrate at 42° C.,
(h) 2× or 4×SSC at 50° C. (low-stringency condition), or
(i) 30 to 40% formamide, 2 x or 4×SSC at 42° C. (low-stringency condition).
   (2) Wash steps can be selected, for example, from the following conditions:
(a) 0.015 M NaCl/0.0015 M sodium citrate/0.1% SDS at 50° C.,
(b) 0.1×SSC at 65° C.,
(c) 0.1×SSC, 0.5% SDS at 68° C.,
(d) 0.1×SSC, 0.5% SDS, 50% formamide at 42° C.,
(e) 0.2×SSC, 0.1% SDS at 42° C.,
(f) 2×SSC at 65° C. (low-stringency condition),

Polypeptides having above-mentioned activity, i.e. conferring the production or the increased production of the fine chemical methionine as compared to a corresponding, e.g. non-transformed, wild type non-human organism, like a microorganism or a plant cell, plant or part thereof, can be encoded by other DNA sequences which hybridize to the sequences shown in the respective line in Table I, application no. 1, columns 5 and 8, preferably the coding region therof, at least under relaxed hybridization conditions and which encode the expression of polypeptides conferring the production or the increased production of the respective fine chemical methionine as compared to a corresponding, e.g. non-transformed, wild type non-human organism, like a microorganism or a plant cell, plant or part thereof.

Further, some applications have to be performed at low stringency hybridization conditions, without any consequences for the specificity of the hybridization. For example, a Southern blot analysis of total DNA could be probed with a nucleic acid molecule of the present invention and washed at low stringency (55° C. in 2×SSPE, 0.1% SDS). The hybridization analysis could reveal a simple pattern of only genes encoding polypeptides of the present invention or used in the process of the invention, e.g. having the herein-mentioned activity of conferring the production or the increased production of the fine chemical as compared to a corresponding, e.g. non-transformed, wild type non-human organism, like a microorganism or a plant cell, plant or part thereof. A further example of such low-stringent hybridization conditions is 4×SSC at 50° C. or hybridization with 30 to 40% formamide at 42° C. Such molecules comprise those which are fragments, analogues or derivatives of the polypeptide of the invention or used in the process of the invention and differ, for example, by way of amino acid and/or nucleotide deletion(s), insertion(s), substitution (s), addition(s) and/or recombination (s) or any other modification (s) known in the art either alone or in combination from the above-described amino acid sequences or their underlying nucleotide sequence(s). However, it is preferred to use high stringency hybridization conditions.

Hybridization should advantageously be carried out with fragments of at least 5, 10, 15, 20, 25, 30, 35 or 40 bp, advantageously at least 50, 60, 70 or 80 bp, preferably at least 90, 100 or 110 bp. Most preferably are fragments of at least 15, 20, 25 or 30 bp. Preferably are also hybridizations with at least 100 by or 200 bp, very especially preferably at least 400 by in length. In an especially preferred embodiment, the hybridization should be carried out with the entire nucleic acid sequence with conditions described above.

The terms “fragment”, “fragment of a sequence” or “part of a sequence” mean a truncated sequence of the original sequence referred to. The truncated sequence (nucleic acid or protein sequence) can vary widely in length; the minimum size being a sequence of sufficient size to provide a sequence with at least a comparable function and/or activity of the original sequence or molecule referred to, or hybridizing with the nucleic acid molecule of the invention or used in the process of the invention under stringent conditions, while the maximum size is not critical. In some applications, the maximum size usually is not substantially greater than that required to provide the desired activity and/or function(s) of the original sequence.

Typically, the truncated amino acid sequence or molecule will range from about 5 to about 310 amino acids in length. More typically, however, the sequence will be a maximum of about 250 amino acids in length, preferably a maximum of about 200 or 100 amino acids. It is usually desirable to select sequences of at least about 10, 12 or 15 amino acids, up to a maximum of about 20 or 25 amino acids.

The term “epitope” relates to specific immunoreactive sites within an antigen, also known as antigenic determinates. These epitopes can be a linear array of monomers in a polymeric composition—such as amino acids in a protein—or consist of or comprise a more complex secondary or tertiary structure. Those of skill will recognize that immunogens (i.e. substances capable of eliciting an immune response) are antigens; however, some antigen, such as haptens, are not immunogens but may be made immunogenic by coupling to a carrier molecule. The term “antigen” includes references to a substance to which an antibody can be generated and/or to which the antibody is specifically immunoreactive.

In an embodiment the present invention relates to an epitope of the polypeptide of the present invention or used in the process of the present invention and conferring the production or the increased production of the fine chemical as compared to a corresponding, e.g. non-transformed, wild type non-human organism, like a microorganism or a plant cell, plant or part thereof.

The term “one or several amino acids” relates to at least one amino acid but not more than that number of amino acids, which would result in a homology of below 50% identity. Preferably, the identity is more than 70%, 80%, 85%, 90%, 91%, 92%, 93%, 94%, 95%, 96%, 97%, 98%, 99% or 99.5% identity.

Further, the nucleic acid molecule of the invention comprises a nucleic acid molecule, which is a complement of one of the nucleotide sequences of above-mentioned nucleic acid molecules or a portion thereof. A nucleic acid molecule or its sequence which is complementary to one of the nucleotide molecules or sequences shown in the respective line in Table I, application no. 1, columns 5 or 8, preferably the coding rgion thereof, homologs or fragments thereof, is one which is sufficiently complementary to one of the nucleotide molecules or sequences shown in the respective line inTable I, application no. 1, columns 5 or 8, preferably the coding rgion thereof, homologs or fragments thereof, such that it can hybridize to one of the nucleotide sequences shown in Table I, application no. 1, columns 5 or 8, preferably the coding rgion thereof, homologs or fragments thereof, thereby forming a stable duplex. Preferably, the hybridization is performed under stringent hybrization conditions. However, a complement of one of the herein disclosed sequences is preferably a sequence complement thereto according to the base pairing of nucleic acid molecules well known to the skilled person. For example, the bases A and G undergo base pairing with the bases T and U or C, resp. and visa versa. Modifications of the bases can influence the base-pairing partner.

The nucleic acid molecule of the invention comprises a nucleotide sequence which is at least about 30%, 35%, 40%, 45%, 50%, 55%, 60%, 65%, 70%, 75%, 80%, 90%, 95%, 96%, 97%, 98%, 99%, 99.5% or more homologous to a nucleotide sequence shown in Table I, application no. 1, columns 5 or 8, preferably the coding region thereof, homologs or fragments thereof, and preferably has above-mentioned activity, in particular conferring the production or the increased production of the fine chemical methionine, respectively, after increasing the activity or an activity of a gene as shown in the respective line in Table I or of a gene product, e.g. as shown in the respective line in Table II, application no. 1, column 5 or 8, by for example in one embodiment expression either in the cytsol or in an organelle such as a plastid or mitochondria or both, preferably in a plastid, or in another embodiment non-targeted or targeted.

The nucleic acid molecule of the invention comprises a nucleotide sequence or molecule which hybridizes, preferably hybridizes under stringent conditions as defined herein, to one of the nucleic acid molecule shown in the respective line in Table I, preferably Table IB, application no. 1, columns 5 or 8, preferably the coding region therof, fragements or homolos therof, and encodes a protein having above-mentioned activity, e.g. conferring the production or the increased production of the respective fine chemical methionine as compared to a corresponding, e.g. non-transformed, wild type non-human organism, like a microorganism or a plant cell, plant or part thereof, by for example in one embodiment expression either in the cytsol or in an organelle such as a plastid or mitochondria or both, preferably in a plastid, or in another embodiment non-targeted or targeted, and optionally, the activity thereof is selected from the group consisting of (DL)-glycerol-3-phosphatase, 2,3-dihydroxyphenylpropionate 1,2-dioxygenase, 2-oxoglutarate dehydrogenase E1 subunit, 49747384_SOYBEAN-protein, 5′-nucleotidase, acetolactate synthase small subunit, acetyl CoA carboxylase, adenosine kinase, arginine exporter protein, Atl g09680-protein, At2g45420-protein, At4g32480-protein, ATP-binding component of a transport system, auxin response factor, b0012-protein, b1003-protein, b1522-protein, b2032-protein, b2345-protein, b2513-protein, b2673-protein, b3246-protein, b3346-protein, b3817-protein, b4029-protein, beta-hydroxylase, bifunctional aspartokinase/homoserine dehydrogenase, calcium-dependent protein kinase, coproporphyrinogen III oxidase, cyclin, cystathionine gamma-synthase, cystathionine-lyase, dihydroxyacid dehydratase, DNA-binding protein, F-box protein, glutaredoxin, glutathione S-transferase, glycogenin, homocitrate synthase, homoserine dehydrogenase, hydrolase, L-serine dehydratase, major facilitator superfamily transporter protein, malic enzyme, membrane transport protein, monothiol glutaredoxin, monthiol glutaredoxin, oxidoreductase, peptidyl-prolyl cis-trans isomerase, phosphoadenosine phosphosulfate reductase , Photosystem I reaction center subunit XI, protein kinase, protein phosphatase, pyruvate kinase, Sec-independent protein translocase subunit, serine protease, serine/threonine-protein phosphatase, threonine aldolase, threonine dehydratase, threonine efflux protein, transcription factor, transport protein, uridine/cytidine kinase, valine-pyruvate transaminase, yhl013c-protein, yml084w-protein, yol160w-protein, yor392w-protein, and zinc finger protein, respectively.

Moreover, the nucleic acid molecule of the invention can comprise only a portion of the coding region of one of the nucleic acid molecules depicted in Table I, application no. 1, columns 5 or 8, for example a fragment which can be used as a probe or primer or a fragment encoding a biologically active portion of the polypeptide of the present invention or of a polypeptide used in the process of the present invention, i.e. having above-mentioned activity, e.g. conferring the production or the increased production of the respective fine chemical methionine as compared to a corresponding, e.g. non-transformed, wild type non-human organism, like a microorganism or a plant cell, plant or part thereof, by for example in one embodiment expression either in the cytsol or in an organelle such as a plastid or mitochondria or both, preferably in a plastid, or in another embodiment non-targeted or targeted. The nucleotide sequences determined from the cloning of the present protein-according-to-the-invention-encoding gene allows for the generation of probes and primers designed for use in identifying and/or cloning its homologues from other cell types and organisms. The probe/primer typically comprises a substantially purified oligonucleotide. The oligonucleotide typically comprises a region of nucleotide sequence that hybridizes under stringent conditions to at least about 12, 15 preferably about 20 or 25, more preferably about 40, 50 or 75 consecutive nucleotides of a sense strand of one of the sequences set forth, e.g., in the respective line in Table I, application no. 1, column 5 or 8, preferably the coding region thereof, or a homolog or a fragment thereof, an anti-sense sequence of one of the sequences, e.g., set forth in the respective line in Table I, application no. 1, columns 5 or 8, preferably the coding region thereof, or a homolog or a fragment thereof or naturally occurring mutants thereof. Primers based on a nucleic acid molecule of the invention can be used in PCR reactions to clone homologs of the polypeptide of the invention or of the polypeptide used in the process of the invention, e.g. as the primers described in the examples of the present invention, e.g. as shown in the examples. A PCR with the primers shown in the respective line in Table III, column 8 will result in a fragment of the gene product as shown in Table II, application no. 1, column 5.

Primer sets are interchangeable. The person skilled in the art knows how to desing and combine said primers to result in the desired product, e.g. in a full length clone or a partial sequence. Probes based on the sequences of the nucleic acid molecule of the invention or used in the process of the present invention can be used to detect transcripts or genomic sequences encoding the same or homologous proteins. The probe can further comprise a label group attached thereto, e.g. the label group can be a radioisotope, a fluorescent compound, an enzyme, or an enzyme co-factor. Such probes can be used as a part of a genomic marker test kit for identifying cells which express a polypeptide of the invention or used in the process of the present invention, such as by measuring a level of an encoding nucleic acid molecule in a sample of cells, e.g. detecting mRNA levels or determining, whether a genomic gene comprising the sequence of the polynucleotide of the invention or used in the processes of the present invention has been mutated or deleted.

As the expression of a respective nucleic acids of the invention is related to the synthesis of the respective fine chemicaL-methionine its function as a probe extends to the detection of microorganisms, plant tissues, plants, plant variets, plant ecotypes or plant genera with varying capability or potential for synthesis of the respective fine chemical methionine. Therefore in one embodiment the present invention relates to a method for analyzing the capability or potential of a plant tissue, a plant, a plant variety or plant ecotype to produce the fine chemicaL-methionine by using the nucleic acid of the invention or parts thereof as a probe to detect the amount of the nucleic acid of the invention in the non-human organism or a part thereof in comparision to another non-human organism.

The nucleic acid molecule of the invention encodes a polypeptide or portion thereof which includes an amino acid sequence which is sufficiently homologous to the amino acid sequence shown in respective line in Table II, application no. 1, columns 5 or 8 such that the protein or portion thereof maintains the ability to confer the production or the increased production of the respective fine chemical methionine as compared to a corresponding, e.g. non-transformed, wild type non-human organism, like a microorganism or a plant cell, plant or part thereof, in particular increasing the activity as mentioned above or as described in the examples in microorganisms or plants.

As used herein, the language “sufficiently homologous” refers to proteins or portions thereof which have amino acid sequences which include a minimum number of identical or equivalent amino acids (e.g. an amino acid which has a similar side chain as an amino acid in one of the sequences of the polypeptide of the present invention) to an amino acid sequence shown in the respective line in Table II, application no. 1, columns 5 or 8 such that the protein or portion thereof is able to confer the production or the increased production of the respective fine chemical methionine as compared to a corresponding, e.g. non-transformed, wild type non-human organism, like a microorganism or a plant cell, plant or part thereof. For example having the activity of a protein as shown in the respective line in Table II, application no. 1, column 3 and as described herein.

In an embodiment of the present invention, the nucleic acid molecule of the present invention comprises a nucleic acid molecule that encodes a portion of the protein of the present invention. The protein is at least about 30%, 35%, 40%, 45% 50%, 55%, 60%, 65%, 70%, 75%, 80%, 85%, 90%, 91%, 92%, 93%, 94%, 95%, 96%, 97%, 98%, 99%, 99.5% or more homologous to an entire amino acid sequence shown in the respective line of Table II, application no. 1, column 5 or 8 and has the above-mentioned activity, e.g. conferring the production or the increased production of the respective fine chemical methionine as compared to a corresponding, e.g. non-transformed, wild type non-human organism, like a microorganism or a plant cell, plant or part thereof by, for example in a embodiment expression either in the cytsol or in an organelle such as a plastid or mitochondria or both, preferably in a plastid, or in another embodiment by targeted or non-targeted expression.

Portions of proteins encoded by the nucleic acid molecule of the invention show preferably the above-mentioned activity, e.g. conferring the production or the increased production of the fine chemical as compared to a corresponding, e.g. non-transformed, wild type non-human organism, like a microorganism or a plant cell, plant or part thereof.

As mentioned herein, the term “biologically active portion” is intended to include a portion, e.g., a domain/motif, that confers the production or the increased production of the fine chemical as compared to a corresponding, e.g. non-transformed, wild type non-human organism, like a microorganism or a plant cell, plant or part thereof or has an immunological activity such that it binds to an antibody binding specifically to the polypeptide of the present invention or a polypeptide used in the process of the present invention for conferring the production or the increased production of the fine chemical as compared to a corresponding, e.g. non-transformed, wild type non-human organism, like a microorganism or a plant cell, plant or part thereof.

The invention further relates to nucleic acid molecules or methods using said nucleic acid molecules that differ from one of the nucleotide sequences shown in the respective line in Table I, application no. 1, columns 5 or 8, preferably the coding region thereof, or fragments or homologs thereof, due to degeneracy of the genetic code but encode a polypeptide of the present invention, in particular a polypeptide having the above-mentioned activity, e.g. as those polypeptides depicted in the respective line in Table II, application no.1, columns 5 or 8, fragments or homologs thereof. Advantageously, the nucleic acid molecule of the invention comprises, or in an other embodiment has, a nucleotide sequence encoding a protein comprising, or in an other embodiment having, an amino acid sequence shown in the respective line in Table II, application no. 1, columns 5 or 8, fragments or homologs thereof. In a further embodiment, the nucleic acid molecule of the invention encodes a full length protein which is substantially homologous to an amino acid sequence shown in the respective line in Table II, application no. 1, columns 5 or 8, or homologs thereof. However, in an embodiment, the nucleic acid molecule of the present invention does not consist of the sequence shown in the respective line in Table I, preferably Table IA, application no. 1, columns 5 or 8, preferably the coding region thereof, but the functional homologs thereof.

In addition, it will be appreciated by those skilled in the art that DNA sequence polymorphisms that lead to changes in the amino acid sequences may exist within a population of the non-human organism of the present invention. Also these non-human organisms are encompassed by the respective non-human organism according to the invention.

As used herein, the terms “gene” and “recombinant gene” refer to nucleic acid molecules comprising an open reading frame encoding the polypeptide of the invention or comprising the nucleic acid molecule of the invention or encoding the polypeptide used in the process of the present invention, preferably from a plant, especially a crop plant, or from a microorgansim useful for the method of the invention. Such genetic polymorphism in the gene encoding the polypeptide of the invention or comprising the nucleic acid molecule of the invention may exist among individuals within a population due to natural variation. Such natural variations can typically result in 1 to 5% variance in the nucleotide sequence of the gene. Any and all such nucleotide variations and resulting amino acid polymorphisms in genes encoding a polypeptide of the invention or comprising a the nucleic acid molecule of the invention that are the result of natural variation and that do not alter the functional activity as described are intended to be within the scope of the invention.

Nucleic acid molecules corresponding to natural variants of a nucleic acid molecule of the invention, which can also be a cDNA, can be isolated based on their homology to the nucleic acid molecules disclosed herein using the nucleic acid molecule of the invention, or a portion thereof, as a hybridization probe according to standard hybridization techniques under stringent hybridization conditions.

Accordingly, in another embodiment, a nucleic acid molecule of the invention is at least 15, 20, 25 or 30 nucleotides in length. Preferably, it hybridizes under stringent conditions to a nucleic acid molecule comprising a nucleotide sequence of the nucleic acid molecule of the present invention or used in the process of the present invention, e.g. comprising the sequence shown in the respective line in Table I, application no. 1, columns 5 or 8, preferably the coding region thereof. In another embodiment the nucleic acid molecule is preferably at least 20, 30, 50, 100, 250 or more nucleotides in length.

The term “hybridizes under stringent conditions” is defined above. In an embodiment, the term “hybridizes under stringent conditions” is intended to describe conditions for hybridization and washing under which nucleotide sequences at least 30%, 40%, 50% or 65% identical to each other typically remain hybridized to each other. Preferably, the conditions are such that sequences at least about 70%, 75%, 80%, 85%, 90% or 95% or more identical to each other typically remain hybridized to each other.

Preferably, a nucleic acid molecule of the invention that hybridizes under stringent conditions to a sequence shown in the respective line in Table I, application no. 1, columns 5 or 8, preferably the coding region thereof, corresponds to a naturally-occurring nucleic acid molecule of the invention. As used herein, a “naturally-occurring” nucleic acid molecule refers to a RNA or DNA molecule having a nucleotide sequence that occurs in nature (e.g. encodes a natural protein). Preferably, the nucleic acid molecule encodes a natural protein having above-mentioned activity, e.g. conferring the production or the increased production of the respective fine chemical methionine as compared to a corresponding, e.g. non-transformed, wild type non-human organism, like a microorganism or a plant cell, plant or part thereof after increasing the expression or activity thereof or the activity of a protein of the invention or used in the process of the invention, in an embodiment for example expression either in the cytsol or in an organelle such as a plastid or mitochondria, preferably in plastids, or, in another embodiment by targeted or non-targeted expression.

In addition to naturally-occurring variants of the sequences of the polypeptide or nucleic acid molecule of the invention as well as of the polypeptide or nucleic acid molecule used in the process of the invention that may exist in the population, the skilled artisan will further appreciate that changes can be introduced by mutation into a nucleotide sequence of the nucleic acid molecule of the invention, e.g. encoding the polypeptide of the invention or used in the process of the present invention, thereby leading to changes in the amino acid sequence of the encoded said polypeptide, without altering the functional ability of the polypeptide, preferably not decreasing said activity, preferably increasing said activity.

For example, nucleotide substitutions leading to amino acid substitutions at “non-essential” amino acid residues can be made in a sequence of the nucleic acid molecule of the invention or used in the process of the invention, e.g. shown in the respective line in Table I, application no. 1, columns 5 or 8, preferably the coding region thereof, or fragments or homologs thereof.

With regard to the activity of a polypeptide a “non-essential” amino acid residue is a residue that can be altered without altering the activity of said polypeptide, whereas an “essential” amino acid residue is required for an activity as mentioned above, e.g. conferring the production or the increased production of the fine chemical as compared to a corresponding, e.g. non-transformed, wild type non-human organism, like a microorganism or a plant cell, plant or part thereof. Other amino acid residues, however, (e.g., those that are not conserved or only semi-conserved in the domain having said activity) may not be essential for activity and thus are likely to be amenable to alteration without altering said activity.

Further, a person skilled in the art knows that the codon usage between organisms can differ. In the enclosed sequence protocol according to WIPO ST 25 the respective donor organism codon usage “translation” is used.The person skilled in the art may adapt the codon usage in the nucleic acid molecule of the present invention to the usage of the organism or the cell compartment for example of the plastid or mitochondria in which the polynucleotide or polypeptide is expressed.

Accordingly, the invention relates to nucleic acid molecules encoding a polypeptide having above-mentioned activity, in an non-human organisms or parts thereof by for example expression either in the cytsol or in an organelle such as a plastid or mitochondria or both, preferably in plastids, that contain changes in amino acid residues that are not essential for said activity. Such polypeptides differ in amino acid sequence from a sequence depicted in the respective line in Table II, especially Table IIA, application no. 1, columns 5 or 8, but retain said activity described herein. The nucleic acid molecule can comprise a nucleotide sequence encoding a polypeptide, wherein the polypeptide comprises an amino acid sequence at least about 50% identical to an amino acid sequence shown in the respective line in Table II, application no. 1, columns 5 or 8, and is capable of conferring the production or the increased production of the respective fine chemical methionine as compared to a corresponding, e.g. non-transformed, wild type non-human organism, like a microorganism or a plant cell, plant or part thereof, after increasing its activity for example in an embodiment by expression either in the cytsol or in an organelle such as a plastid or mitochondria or both, preferably in plastids, or, in another embodiment by targeted or non-targeted expression. Preferably, the protein encoded by the nucleic acid molecule is at least about 60%, 70%, 80%, 90%, 95%, 96%, 97%, 98%, 99% or 99.5% identical to the sequence shown in the respective line in Table II, application no. 1, columns 5 or 8.

To determine the percentage identity (=homology, herein used interchangeably) of two amino acid sequences or of two nucleic acid molecules, the sequences are written one underneath the other for an optimal comparison (for example gaps may be inserted into the sequence of a protein or of a nucleic acid in order to generate an optimal alignment with the other protein or the other nucleic acid). The amino acids or nucleobases at the respective corresponding positions are then compared. If a position in one sequence is occupied by the same amino acid or the same nucleobase as the corresponding position in the other sequence, the molecules are homologous at this position. The percentage identity between the two sequences is a function of the number of identical positions shared by the sequences (i.e. % homology=% identity=number of identical positions/total number of positions×100).

For the determination of the percentage identity (=homology) of two or more amino acid sequences or of two or more nucleic acid molecules several computer software programs have been developed. The homology of two or more sequences can be calculated with for example the software fasta, which presently has been used in the version fasta 3 (Pearson W. R. and Lipman D. J., PNAS 85, 2444 (1988); Pearson W.R., Methods in Enzymology 183, 63 (1990)). Another useful program for the calculation of homologies of different sequences is the standard blast program, which is included in the Biomax PEDANTTM software (Biomax, Munich, Federal Republic of Germany). This leads unfortunately sometimes to suboptimal results since blast does not always include complete sequences of the subject and the querry. Nevertheless as this program is very efficient it can be used for the comparison of a huge number of sequences. The following settings are typically used for such a comparison of sequences: -p Program Name [String]; -d Database [String]; default=nr; -i Query File [File In]; default=stdin; -e Expectation value (E) [Real]; default=10.0; -m alignment view options: 0=pairwise; 1=query-anchored showing identities; 2=query-anchored no identities; 3=flat query-anchored, show identities; 4=flat query-anchored, no identities; 5=query-anchored no identities and blunt ends; 6=flat query-anchored, no identities and blunt ends; 7=XML Blast output; 8=tabular; 9 tabular with comment lines [Integer]; default =0; -o BLAST report Output File [File Out] Optional; default=stdout; -F Filter query sequence (DUST with blastn, SEG with others) [String]; default=T; -G Cost to open a gap (zero invokes default behavior) [Integer]; default=0; -E Cost to extend a gap (zero invokes default behavior) [Integer]; default=0; -X X dropoff value for gapped alignment (in bits) (zero invokes default behavior); blastn 30, megablast 20, tblastx 0, all others 15 [Integer]; default=0; -I Show GI's in deflines [T/F]; default =F; -q Penalty for a nucleotide mismatch (blastn only) [Integer]; default=-3; -r Reward for a nucleotide match (blastn only) [Integer]; default=1; -v Number of database sequences to show one-line descriptions for (V) [Integer]; default=500; -b Number of database sequence to show alignments for (B) [Integer]; default=250; -f Threshold for extending hits, default if zero; blastp 11, blastn 0, blastx 12, tblastn 13; tblastx 13, megablast 0 [Integer]; default=0; -g Perfom gapped alignment (not available with tblastx) [T/F]; default =T; -Q Query Genetic code to use [Integer]; default=1; -D DB Genetic code (for tblast[nx] only) [Integer]; default=1; -a Number of processors to use [Integer]; default=1; -O SeqAlign file [File Out] Optional; -J Believe the query defline [T/F]; default=F; -M Matrix [String]; default=BLOSUM62; -W Word size, default if zero (blastn 11, megablast 28, all others 3) [Integer]; default=0; -z Effective length of the database (use zero for the real size) [Real]; default=0; -K Number of best hits from a region to keep (off by default, if used a value of 100 is recommended) [Integer]; default=0; -P 0 for multiple hit, 1 for single hit [Integer]; default=0; -Y Effective length of the search space (use zero for the real size) [Real]; default=0; -S Query strands to search against database (for blast[nx], and tblastx); 3 is both, 1 is top, 2 is bottom [Integer]; default=3; -T Produce HTML output [T/F]; default=F; -I Restrict search of database to list of GI's [String] Optional; -U Use lower case filtering of FASTA sequence [T/F] Optional; default=F; -y X dropoff value for ungapped extensions in bits (0.0 invokes default behavior); blastn 20, megablast 10, all others 7 [Real]; default=0.0; -Z X dropoff value for final gapped alignment in bits (0.0 invokes default behavior); blastn/megablast 50, tblastx 0, all others 25 [Integer]; default =0; -R PSI-TBLASTN checkpoint file [File In] Optional; -n MegaBlast search [T/F]; default=F; -L Location on query sequence [String] Optional; -A Multiple Hits window size, default if zero (blastn/megablast 0, all others 40 [Integer]; default=0; -w Frame shift penalty (00F algorithm for blastx) [Integer]; default =0; -t Length of the largest intron allowed in tblastn for linking HSPs (0 disables linking) [Integer]; default=0.

Results of high quality are reached by using the algorithm of Needleman and Wunsch or Smith and Waterman. Therefore programs based on said algorithms are preferred. Advantageously the comparisons of sequences can be done with the program PileUp (J. Mol.

Evolution., 25, 351 (1987), Higgins et al., CABIOS 5, 151 (1989)) or preferably with the programs “Gap” and “Needle”, which are both based on the algorithms of Needleman and Wunsch (J. Mol. Biol. 48, 443 (1970)), and “BestFit”, which is based on the algorithm of Smith and Waterman (Adv. Appl. Math. 2, 482 (1981)). “Gap” and “BestFit” are part of the GCG software-package (Genetics Computer Group, 575 Science Drive, Madison, Wis., USA 53711 (1991); Altschul et al., (Nucleic Acids Res. 25, 3389 (1997)), “Needle” is part of the The European Molecular Biology Open Software Suite (EMBOSS) (Trends in Genetics 16 (6), 276 (2000)). Therefore preferably the calculations to determine the percentages of sequence homology are done with the programs “Gap” or “Needle” over the whole range of the sequences. The following standard adjustments for the comparison of nucleic acid sequences were used for “Needle”: matrix: EDNAFULL, Gap_{—penalty:} 10.0, Extend_{—penalty:} 0.5. The following standard adjustments for the comparison of nucleic acid sequences were used for “Gap”: gap weight: 50, length weight: 3, average match: 10.000, average mismatch: 0.000.

For example a sequence, which has 80% homology with sequence SEQ ID

NO: 69 at the nucleic acid level is understood as meaning a sequence which, upon comparison with the sequence SEQ ID NO: 69 by the above-mentioned program “Needle” with the above-mentioned parameter set, has a 80% homology.

Homology between two polypeptides is understood as meaning the identity of the amino acid sequence over in each case the entire sequence length which is calculated by comparison with the aid of the above program “Needle” using Matrix: EBLOSUM62, Gap_penalty: 8.0, Extend_penalty: 2.0.

For example a sequence which has a 80% homology with sequence SEQ ID NO: 70 at the protein level is understood as meaning a sequence which, upon comparison with the sequence SEQ ID NO: 70 by the above-mentioned program “Needle” with the above-mentioned parameter set, has a 80% homology.

Functional equivalents of the nucleic acid molecules according to the present invention and/or used in the process according to the present invention represent an embodiment of homologs. Functional equivalents derived from the nucleic acid molecules as shown in the respective line in Table I, application no. 1, columns 5 or 8, preferably the coding region thereof, according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the nucleic acid molecules as shown in the respective line in Table I, application no. 1, columns 5 or 8, preferably the coding region thereof, according to the invention and encode polypeptides having essentially the same properties as the polypeptide as shown in the respective line in Table II, application no. 1, columns 5 or 8.

Functional equivalents derived from one of the polypeptides as shown in the respective line in

Table II, application no. 1, columns 5 or 8 according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the polypeptides as shown in the respective line in Table II, application no. 1, columns 5 or 8 according to the invention and having essentially the same properties as the polypeptide as shown in the respective line in Table II, application no. 1, columns 5 or 8.

“Essentially the same properties” of a functional equivalent is above all understood as meaning that the functional equivalent has the above-mentioned activity, by for example in an embodiment expression either in the cytsol or in an organelle such as a plastid or mitochondria or both, or, in another embodiment by targeted or non-targeted expression, while increasing the amount of protein, activity or function of said functional equivalent in an organism, e.g. a microorgansim, a plant or plant tissue or animal tissue, plant or animal cells or a part of the same.

A nucleic acid molecule encoding a homolog to a polypeptide depicted in the respective line in Table II, application no. 1, columns 5 or 8 can be created by introducing one or more nucleotide substitutions, additions or deletions into the nucleic acid molecule of the present invention, in particular of Table I, application no. 1, columns 5 or 8, in the respective line, such that one or more amino acid substitutions, additions or deletions are introduced into the encoded protein. Mutations can be introduced into the nucleic acid molecules as depicted in the respective line in Table I, application no. 1, columns 5 or 8, preferably the coding region thereof, by standard techniques, such as site-directed mutagenesis,PCR-mediated mutagenesis or other methods known to the person skilled in the art.

Preferably, conservative amino acid substitutions are made at one or more predicted non-essential amino acid. A “conservative amino acid substitution” is one in which the amino acid is replaced with an amino acid having a similar side chain. Families of amino acids having similar side chains have been defined in the art. These families include amino acids with basic side chains (e.g. lysine, arginine, histidine), acidic side chains (e.g. aspartic acid, glutamic acid), uncharged polar side chains (e.g. glycine, asparagine, glutamine, serine, threonine, tyrosine, cysteine), nonpolar side chains (e.g. alanine, valine, leucine, isoleucine, proline, phenylalanine, methionine, tryptophane), beta-branched side chains (e.g. threonine, valine, isoleucine) and aromatic side chains (e.g. tyrosine, phenylalanine, tryptophane, histidine).

Thus, a predicted non-essential amino acid in a polypeptide of the invention or a polypeptide used in the process of the invention is preferably replaced with another amino acid from the same family. Alternatively, in another embodiment, mutations can be introduced randomly along all or part of a coding sequence of a nucleic acid molecule of the invention or used in the process of the invention, such as by saturation mutagenesis, and the resultant mutants can be screened for activity described herein to identify mutants that retain or even have increased activity, e.g. conferring the production or the increased production of the fine chemical as compared to a corresponding, e.g. non-transformed, wild type non-human organism, like a microorganism or a plant cell, plant or part thereof.

Following mutagenesis of one of the sequences as shown herein, the encoded protein can be expressed recombinantly and the activity of the protein can be determined using, for example, assays described herein (see Examples) or by measuring the resulting fine chemical production in an organsimen expressing the mutagenized form of the sequence in comparison to an organsim expressing the original non-mutagenized form of the sequence.

Homologues of the nucleic acid sequences used, with the sequences shown in the respective line in Table I, application no. 1, columns 5 or 8, perferably the coding region thereof, comprise also allelic variants with at least approximately 30%, 35%, 40%, 45%, 50%, 55%, 60%, 65%, 70%, 75%, 80%, 85%, 90%, 91%, 92%, 93%, 94% 95%. 96%, 97%, 98%, 99%, 99.5% or more % homology with one of the nucleotide sequences shown or the above-mentioned derived nucleic acid sequences or their homologues or derivatives or parts of these. Allelic variants encompass in particular functional variants which can be obtained by deletion, insertion or substitution of nucleotides from the sequences shown in the respective line, preferably in Table I, columns 5 or 8, preferably the coding region theeof, or from the derived nucleic acid sequences, the intention being, however, that the enzymatic activity or the biological activity of the resulting proteins synthesized is advantageously retained or increased.

In one embodiment of the present invention, the nucleic acid molecule of the invention or used in the process of the invention comprises the sequences shown in the respective line in any of the Table I, application no. 1, columns 5 or 8, preferably the coding region thereof, fragments or homologs thereof. It is preferred that the nucleic acid molecule comprises as little as possible other nucleotides not shown in the respective line in any one of Table I, application no. 1, columns 5 or 8, preferably the coding region thereof, fragments or homologs thereof. In one embodiment, the nucleic acid molecule comprises less than 500, 400, 300, 200, 100, 90, 80, 70, 60, 50 or 40 further nucleotides. In a further embodiment, the nucleic acid molecule comprises less than 30, 20 or 10 further nucleotides. In one embodiment, the nucleic acid molecule used in the process of the invention is identical to the sequences shown in the respective line in Table I, application no. 1, columns 5 or 8, preferably the coding region thereof.

Also preferred is that the nucleic acid molecule used in the process of the invention encodes a polypeptide comprising the sequence shown in the respective line in Table II, application no. 1, columns 5 or 8, fragments or homologs thereof. In one embodiment, the nucleic acid molecule encodes less than 150, 130, 100, 80, 60, 50, 40 or 30 further amino acids. In a further embodiment, the encoded polypeptide comprises less than 20, 15, 10, 9, 8, 7, 6 or 5 further amino acids. In one embodiment used in the inventive process, the encoded polypeptide is identical to the sequences shown in the respective line in Table II, application no. 1, columns 5 or 8.

In one embodiment, the nucleic acid molecule of the invention or used in the process encodes a polypeptide comprising the sequence shown in the respective line in Table II, application no. 1, columns 5 or 8, or fragments or homologs thereof, and comprises less than 100 further nucleotides. In a further embodiment, said nucleic acid molecule comprises less than 30 further nucleotides. In one embodiment, the nucleic acid molecule used in the process is identical to a coding sequence of the sequences shown in the respective line in Table I, application no. 1, columns 5 or 8.

Polypeptides (=proteins), which still have the essential biological or enzymatic activity of the polypeptide of the present invention conferring the production or the increased production of the respective fine chemical methionine as compared to a corresponding, e.g. non-transformed, wild type non-human organism, like a microorganism or a plant cell, plant or part thereof, i.e. whose activity is essentially not reduced, are polypeptides with at least 10%, 20%, 30%, 40%, 50%, 60%, 70%, 80%, 90%, 95% or more of the wild type biological activity or enzymatic activity, advantageously, the activity is essentially not reduced in comparison with the activity of a polypeptide shown in the respective line in Table II, application no.1, columns 5 or 8 expressed under identical conditions.

Homologues of nucleic acid molecules shown in the respective line in Table I, application no. 1, columns 5 or 8, preferably the coding region thereof, or fragments thereof, or of the derived sequences shown in the respective line in Table II, application no. 1, columns 5 or 8, or fragments thereof, also mean truncated sequences, cDNA, single-stranded DNA or RNA of the coding and noncoding DNA sequence. Homologues of said sequences are also understood as meaning derivatives, which comprise noncoding regions such as, for example, UTRs, introns, terminators, enhancers or promoter variants. The promoters upstream of the nucleotide sequences stated can be modified by one or more nucleotide substitution(s), insertion(s) and/or deletion(s) without, however, interfering with the functionality or activity either of the promoters, the open reading frame (=ORF) or with the 3′-regulatory region such as terminators or other 3′-regulatory regions, which are far away from the ORF. It is furthermore possible that the activity of the promoters is increased by modification of their sequence, or that they are replaced completely by more active promoters, even promoters from heterologous organisms.

Appropriate promoters are known to the person skilled in the art and are mentioned herein below.

Accordingly, one embodiment of the invention relates to a host cell, which has been transformed stably or transiently with the vector according to the invention or the nucleic acid molecule according to the invention or the nucleic acid construct or the expression construct according to the invention.

Depending on the non-human host organism, the non-human organisms used in the process according to the invention are cultured or grown in a manner with which the skilled worker is familiar.

As a rule, microorganisms are grown in a liquid medium comprising a carbon source, usually in the form of sugars, a nitrogen source, usually in the form of organic nitrogen sources such as yeast extract or salts such as ammonium sulfate, trace elements such as iron salts, manganese salts, magnesium salts, and, if appropriate, vitamins, at temperatures between 0° C. and 100° C., preferably between 10° C. and 60° C., while passing in oxygen. In the event the microorganism is anaerobe, no oxygen is blown through the culture medium. The pH value of the liquid nutrient medium may be kept constant, that is to say regulated during the culturing phase, or not. The organisms may be cultured batchwise, semibatchwise or continuously. Nutrients may be provided at the beginning of the fermentation or fed in semicontinuously or continuously. Advantageously microorganisms such as algae are grown under sunlight in open ponds or in fermentors illuminated with a light intensity between 10 to 2000 μmol×m⁻²33 s⁻¹, preferred between 100 to 1000 μmol×m⁻²×s^{-1,} more preferred between 200 to 800 μmol×m⁻²×s⁻¹, most preferred between 300 to 600 μmol×m⁻² ×s⁻¹. The cells are grown between several hours for example 3 to 48 h and several days 1 to 20 days, preferably 2 to 10 days. Algae as autotrophic organisms grow well in the presence of light as energy source, anorganic hydrogen donors and CO₂ as sole carbon source.

As a rule, plants used in the process according to the invention are grown according to general knowledge of a skilled worker.

The fine chemical produced can be isolated from the non-human organism by methods with which the skilled worker is familiar. For example via extraction, salt precipitation or chromatography, like ion-exchange chromatography. To this end, the non-human organisms, especially the microorganism or the plant, may be disrupted beforehand. The process according to the invention can be conducted batchwise, semibatchwise or continuously. A summary of known culture and isolation techniques can be found in the textbook by Chmiel (“Bioprozeβtechnik 1, Einführung in die Bioverfahrenstechnik”, Gustav Fischer Verlag, Stuttgart, 1991)), Demain et al. (“Industrial Microbiology and Biotechnology”, 2nd edition, ASM Press, Washington, D.C., 1999, ISBN 1-55581-128-0) or in the textbook by Storhas (“Bioreaktoren and periphere Einrichtungen”, Vieweg Verlag, Braunschweig/Wiesbaden, 1994)).

In one embodiment, the present invention relates to a polypeptide encoded by the nucleic acid molecule according to the present invention, preferably conferring the production of or an increase in the fine chemical content in an non-human organism or cell after generating or increasing the expression or activity of said nucleic acid molecule, eithet in the cytosol or in an organelles such as a plastid or mitochondrion or both, preferably in a plastid, or in another embodiment by targeted or non-targeted expression.

The present invention also relates to a process for the production of a polypeptide according to the present invention, the polypeptide being expressed in a host cell according to the invention, preferably in a microorganism or a plant cell, plant or a part thereof, especially in a transgenic microorganism or a transgenic plant cell, plant or a part thereof.

In an embodiment, the nucleic acid molecule used in the process for the production of the polypeptide (FCRP) is derived from a microorganism such as a eukaryotic or prokaryotic microorganism, preferably from a eukaryotic microorganism, such as an algae or Saccharomyces cerevisiae; in an embodiment thereof this polypeptide is produced in another microorganism or in another embodiment thereof this polypeptide is produced in a plant cell, plant or a part thereof. In another embodiment, the nucleic acid molecule used in the process for the production of the polypeptide is derived from a plant, in an embodiment thereof the polypeptide is produced in a microorganism or in another embodiment thereof this polypeptide is produced in another plant, a part or cell thereof.

The skilled worker knows that protein and DNA expressed in different non-human organisms differ in many respects and properties,e. g. DNA modulation and imprinting, such as methylation or post-translational modification, as for example glucosylation, phosphorylation, acetylation, myristoylation, ADP-ribosylation, farnesylation, carboxylation, sulfation, ubiquination, etc. though having the same coding sequence. Preferably, the cellular expression control of the corresponding protein differs accordingly in the control mechanisms controlling the activity and expression of an endogenous protein or another prokaryotic or eukaryotic protein. One major difference between proteins expressed in prokaryotic or eukaryotic organisms is the amount and pattern of glycosylation. For example in E. coli there are no glycosylated proteins. Proteins expressed in yeasts have a high mannose content in the glycosylated proteins, whereas in plants the glycosylation pattern is complex.

The polypeptide (FCRP) of the present invention is preferably produced by recombinant DNA techniques. For example, a nucleic acid molecule encoding the protein is cloned into a vector (as described above), the vector is introduced into a host cell (as described above) and said polypeptide is expressed in the host cell. Said polypeptide (FCRP) can then be isolated from the cells by an appropriate purification scheme using standard protein purification techniques. Alternative to recombinant expression, the polypeptide or peptide of the present invention can be synthesized chemically using standard peptide synthesis techniques.

Moreover, a native polypeptide conferring the increase of the respective fine chemical methionine in a non-human organism or a part thereof can be isolated from cells (e.g., endothelial cells), for example using the antibody of the present invention as described below, in particular, an antibody against proteins having (DL)-glycerol-3-phosphatase, 2,3-dihydroxyphenylpropionate 1,2-dioxygenase, 2-oxoglutarate dehydrogenase E1 subunit, 49747384_SOYBEAN-protein, 5′-nucleotidase, acetolactate synthase small subunit, acetyl CoA carboxylase, adenosine kinase, arginine exporter protein, At1g09680-protein, At2g45420-protein, At4g32480-protein, ATP-binding component of a transport system, auxin response factor, b0012-protein, b1003-protein, b1522-protein, b2032-protein, b2345-protein, b2513-protein, b2673-protein, b3246-protein, b3346-protein, b3817-protein, b4029-protein, beta-hydroxylase, bifunctional aspartokinase/homoserine dehydrogenase, calcium-dependent protein kinase, coproporphyrinogen III oxidase, cyclin, cystathionine gamma-synthase, cystathionine-lyase, dihydroxyacid dehydratase, DNA-binding protein, F-box protein, glutaredoxin, glutathione S-transferase, glycogenin, homocitrate synthase, homoserine dehydrogenase, hydrolase, L-serine dehydratase, major facilitator superfamily transporter protein, malic enzyme, membrane transport protein, monothiol glutaredoxin, monthiol glutaredoxin, oxidoreductase, peptidyl-prolyl cistrans isomerase, phosphoadenosine phosphosulfate reductase , Photosystem I reaction center subunit XI, protein kinase, protein phosphatase, pyruvate kinase, Sec-independent protein translocase subunit, serine protease, serine/threonine-protein phosphatase, threonine aldolase, threonine dehydratase, threonine efflux protein, transcription factor, transport protein, uridine/cytidine kinase, valine-pyruvate transaminase, yhl013c-protein, yml084w-protein, yol160w-protein, yor392w-protein, or zinc finger protein activity, respectively, or an antibody against polypeptides as shown in the respective line in Table II, application no. 1, columns 5 or 8, or fragments or homologs thereof which can be produced by standard techniques utilizing the polypeptid of the present invention or fragment thereof, i.e., the polypeptide of this invention (FCRP). Preferred are monoclonal antibodies.

In an embodiment, the present invention relates to a polypeptide encoded by a nucleic acid molecule of the invention or obtainable by a process of the invention. Said polypeptide confers preferably the aforementioned activity, in particular, the polypeptide confers the generation or the increased production of the fine chemical in a cell or an organsim or a part thereof after generating or increasing the cellular activity, e.g. by generating or increasing the expression or the specific activity of the polypeptide.

In one embodiment, the present invention relates to a polypeptide as depicted in the respective line in Table II, especially in Table IIA or especially in Table IIB, application no. 1, columns 5 or 8, or fragment or homolog thereof or as coded by the nucleic acid molecule shown in the respective line in Table I, especially Table IA or especially Table IB, application no. 1, columns 5 or 8, preferably the coding region thereof, or fragment or homolog thereof.

In an embodiment, the present invention relates to a polypeptide comprising or consisting of a consensus sequence shown in the respective line in Table IV, application no. 1, column 8 or at least one polypeptide motif shown in the respective line in Table IV, application no. 1, column 8.

In an embodiment thereof, the present invention relates to a polypeptide comprising or consisting of a consensus sequence shown in the respective line in Table IV, application no. 1, column 8 or comprising at least one polypeptide motif shown in the respective line in Table IV, application no. 1, column 8, whereby not more than 20, 15, 10, 9, 8, 7, 6, 5, 4, 3, 2, or 1, or 0 of the amino acids can be replaced by any amino acid.

In another embodiment thereof, the present invention relates to a polypeptide comprising or consisting of a consensus sequence shown in the respective line in Table IV, application no. 1, column 8 or comprising at least one polypeptide motif shown in the respective line in Table IV, application no. 1, column 8, whereby not more than 15%, 10%, 5%, 4%, 3%, 2% or1% or 0% of the amino acids defined as distinct specific amino acids are/is replaced by another amino acid.

In another embodiment thereof, the present invention relates to a polypeptide comprising or consisting of a consensus sequence shown in the respective line in Table IV, application no. 1, column 8 or comprising at least one polypeptide motif shown in the respective line in Table IV, application no. 1, column 8, whereby not more than 20, 15, 10, 9, 8, 7, 6, 5, 4, 3, 2, or 1, or 0 amino acids are inserted into the consensus sequence or polypeptide motif.

In an embodiment of the present invention, the polypeptide of the present invention comprises or consists of a consensus sequence or at least one polypeptide motif shown in the respective line in Table IV, application no. 1, column 8, whereby said polypeptide distinguishes over the sequence depicted in the respective line in Table II, application no. 1, columns 5 or 8 by one or more amino acids. In one embodiment, said polypeptide of the invention distinguishes over the sequence shown in the respective line in Table II, application no. 1, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 1, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in the respective line in Table II, application no. 1, columns 5 and 8.

In an embodiment, the invention relates to a polypeptide conferring an increase in the respective fine chemical methionine in a non-human organism, especially a microorganism or a plant, or a part thereof, being encoded by the nucleic acid molecule of the invention or used in the process of the invention and having a sequence which distinguishes over the sequence as shown in the respective line in Table II, application no. 1, columns 5 or 8 by one or more amino acids (but not exceeding 5%, preferably 4%, 3%, 2%, 1%, 0.5%, 0.2% or 0.1%). In an embodiment, said polypeptide of the invention does not comprise or consist of the sequence shown in the respective line in Table II, application no. 1, columns 5 or 8. In an embodiment, said polypeptide of the present invention is less than 100%, 99.999%, 99.99%, 99.9% or 99% identical. In one embodiment, said polypeptide which differs at least in one or more amino acids (but not exceeding 5%, preferably 4%, 3%, 2%, 1%, 0.5%, 0.2% or 0.1%) from the polypeptide shown in the respective line in Table II, application no. 1, columns 5 and 8 does not comprise a protein of the sequence shown in the respective line in Table II A and/or II B, application no. 1, columns 5 or 8.

In an embodiment, the present invention relates to a polypeptide having the activity of the protein as shown in the respective line in Table II, application no. 1, column 3, which distinguishes over the sequence depicted in the respective line in Table II, application no. 1, columns 5 or 8 by one or more amino acids, preferably by more than 5, 6, 7, 8 or 9 amino acids, preferably by more than 10, 15, 20, 25 or 30 amino acids, evenmore preferred are more than 40, 50, or 60 amino acids and, preferably, the sequence of the polypeptide of the invention distinguishes from the sequence shown in the respective line in Table II, application no. 1, columns 5 and 8 by not more than 80%, 70%, 60%, 50%, 40%, 30%, 20%, 10%, 5%, 4%, 3%, 2%, 1% or 0.5%.

In another embodiment the polypeptide of the invention takes the form of a preprotein consisting of a plastidial or mitochondrial transit peptide joint to a polypeptide having the activity of the protein as shown in Table II, column 3, from which the transit peptide is preferably cleaved off upon transport of the preprotein into the organelle for example into the plastid or mitochondrion.

In another embodiment the polypeptide of the invention takes not the form of a preprotein.

The terms “protein” and “polypeptide” used in this application are interchangeable. “Polypeptide” refers to a polymer of amino acids (amino acid sequence) and does not refer to a specific length of the molecule. Thus peptides and oligopeptides are included within the definition of polypeptide. This term does also refer to or include post-translational modifications of the polypeptide, for example, glycosylations, acetylations, phosphorylations and the like. Included within the definition are, for example, polypeptides containing one or more analogs of an amino acid (including, for example, unnatural amino acids, etc.), polypeptides with substituted linkages, as well as other modifications known in the art, both naturally occurring and non-naturally occurring.

Another aspect of the present invention pertains to isolated FCRP, or biologically active portions thereof. An “isolated” or “purified” protein or biologically active portion thereof is substantially free of cellular material when produced by recombinant DNA techniques or chemical precursors or other chemicals when chemically synthesized.

The language “substantially free of cellular material” includes preparations of the polypeptide of the invention in which the protein is separated from cellular components of the cells in which it is naturally or recombinantly produced. In an embodiment, the language “substantially free of cellular material” includes preparations having less than about 30% (by dry weight) of “contaminating protein”, more preferably less than about 20% of “contaminating protein”, still more preferably less than about 10% of “contaminating protein”, and most preferably less than about 5% “contaminating protein”. The term “contaminating protein” relates to polypeptides, which differ from the polypeptide in question of the present invention. When the polypeptide of the present invention or biologically active portion thereof is recombinantly produced, it is also preferably substantially free of culture medium, i.e., culture medium represents less than about 20%, more preferably less than about 10%, and most preferably less than about 5% of the volume of the protein preparation. The language “substantially free of chemical precursors or other chemicals” includes preparations in which the polypeptide of the present invention is separated from chemical precursors or other chemicals, which are involved in the synthesis of the protein. The language “substantially free of chemical precursors or other chemicals” includes preparations having less than about 30% (by dry weight) of chemical precursors, other chemicals or other proteins, which differ from the polypeptide in question. Other chemical precursors, other chemicals or other proteins, which are not identical to the proteins as shown in the respective line in Table II, column 5 or 8, or fragments or homologs thereof, are all collectevly named as impurities. The term “chemical precursors” shall mean in the sense of the specification chemical substances, which are intermediates of the biochemical pathway within the organism or within the cell(s) of the non-human organism for example glucose-6-phoshat, citrate, fumarate, homoserine etc. The term “other chemicals” shall mean in the sense of the specification chemical substances, which are endproducts of the biochemical pathway within the organism or within the cell(s) of the organism for example amino acids such as lysine, alanine etc; fatty acids such as linolenic acid, eicosapantaenoic acid etc, sugars such as glucose, mannose, ribose, desoxy ribose etc, vitamins such as vitamin C, vitamin B2 etc. and all other chemical substances of the cell. The term “other proteins” shall mean in the sense of the specification all other proteins, which are not identical to the proteins mentioned in the respective line in Table II, columns 5 or 8, or fragments or homologs thereof. The fine chemical preparations advantageously shall have less than about 25% impurities, preferably less than about 20% impurities, still more preferably less than about 10% impurities, and most preferably less than about 5% impurities. In preferred embodiments, isolated proteins or biologically active portions thereof lack contaminating proteins from the same organism from which the polypeptide of the present invention is derived. Typically, such proteins are produced by recombinant techniques.

A polypeptide of the invention can participate in the process of the present invention. The polypeptide or a portion thereof comprises preferably an amino acid sequence, which is sufficiently homologous to an amino acid sequence shown in the respective line in Table II A and/or II B, application no. 1, columns 5 or 8.

Further, the polypeptide can have an amino acid sequence which is encoded by a nucleotide sequence which hybridizes, preferably hybridizes under stringent conditions as described above, to a nucleotide sequence of the nucleic acid molecule of the present invention. Accordingly, the polypeptide has an amino acid sequence which is encoded by a nucleotide sequence that is at least about 35%, 40%, 45%, 50%, 55%, 60%, 65%, 70%, 75%, 80%, 85%, 90%, 91%, 92%, 93%, 94%, 95%, 96%, 97%, 98%, 99%, 99.5% or more homologous to one of the amino acid sequences as shown in the respective line in Table II A and/or II B, application no. 1, columns 5 or 8, or fragments thereof. The preferred polypeptide of the present invention preferably possesses at least one of the activities according to the invention and described herein. A preferred polypeptide of the present invention includes an amino acid sequence encoded by a nucleotide sequence which hybridizes, preferably hybridizes under stringent conditions, to a nucleotide sequence shown in the respective line in Table I A and/or I B, application no. 1, columns 5 or 8, preferably the coding region thereof, or fragments thereto or which is homologous thereto, as defined above.

The polypeptide of the present invention can vary from the sequences shown in the respective line in Table II A and/or II B, application no. 1, columns 5 or 8 in amino acid sequence due to natural variation or mutagenesis, as described in detail herein. Accordingly, the polypeptide comprise an amino acid sequence which is at least about 35%, 40%, 45%, 50%, 55%, 60%, 65%, 70%, 75%, 80%, 85% or 90, 91%, 92%, 93%, 94%, 95%, 96%, 97%, 98%, 99%, 99.5% or more homologous to an entire amino acid sequence shown in the respective line in Table II A and/or II B, application no. 1, columns 5 or 8.

Biologically active portions of a polypeptide of the present invention include peptides comprising amino acid sequences derived from the amino acid sequence of the polypeptide of the present invention or used in the process of the present invention, e.g., the amino acid sequence shown in the respective line in Table II, application no. 1, columns 5 or 8 or the amino acid sequence of a protein homologous thereto, which include fewer amino acids than a full length polypeptide of the present invention or used in the process of the present invention or the full length protein which is homologous to a polypeptide of the present invention or used in the process of the present invention depicted herein, and exhibit at least one activity of a polypeptide of the present invention or used in the process of the present invention.

Typically, biologically (or immunologically) active portions of i.e. peptides are, for example, 5, 10, 15, 20, 30, 35, 36, 37, 38, 39, 40, 50, 75, 100, 125, 150 or more amino acids in length; especially they comprise a domain or motif with at least one activity or epitope of a polypeptide of the present invention or used in the process of the present invention. Moreover, other biologically active portions, in which other regions of the polypeptide are deleted, can be prepared by recombinant techniques and evaluated for one or more of the activities described herein.

Manipulation of the nucleic acid molecule of the invention may result in the production of a protein having differences from the protein as shown in the respective line in

Table II, application no. 1, column 5 or 8. Differences shall mean at least one amino acid different from the sequences as shown in the respective line in Table II, application no. 1, column 5 or 8, preferably at least 2, 3, 4, 5, 6, 7, 8, 9 or 10 amino acids (especially but not exceeding 0.1%, preferably 0.2%, 0.5%, 1%, 2%, 3% 4% or 5%), more preferably at least 15, 20, 25, 30, 35, 40, 45 or 50 amino acids (especially but not exceeding 0.5%, preferably 1%, 2%, 3% 4%, 5%, 10% or 25%) different from the sequences as shown in the respective line in Table II, application no. 1, column 5 or 8. These proteins may be improved in efficiency or activity, or may be more stable and therefore present in greater numbers in the cell as compared to the wild-type cell.

Any mutagenesis strategies for the polypeptide of the present invention or the polypeptide used in the process of the present invention to result in increasing said activity are not meant to be limiting; variations on these strategies will be readily apparent to one skilled in the art. Using such strategies, and incorporating the mechanisms disclosed herein, the nucleic acid molecule and polypeptide of the invention may be utilized to generate algae, ciliates, fungi, other microorganisms like C. glutamicum or plants or parts thereof, expressing proteins as shown in the respective line in Table II, application no. 1, column 5 or 8, or mutated proteins thereof. The nucleic acid molecules and polypeptide molecules of the invention are expressed such that the yield, production, and/or efficiency of production of a desired compound is improved.

This desired compound may be any natural product of the respective non-human organism, especially a microorganism or a plant, which includes the final products of biosynthesis pathways and intermediates of naturally-occurring metabolic pathways, as well as molecules which do not naturally occur in the metabolism of said cells of said non-human organism, but which are produced by said cells of the invention.

The invention also provides chimeric or fusion proteins.

As used herein, a “chimeric protein” or “fusion protein” comprises a polypeptide of the present invention operatively linked to a polypeptide whereby the latter does not confer on its own above-mentioned activity, in particular, which does not confer an increase of content of the fine chemical in a cell or a non-human organism or a part thereof, if its activity is increased.

In one embodiment, a protein (=polypeptide) as shown in the respective line in Table II, application no. 1, column 5 or 8 refers to a polypeptide having an amino acid sequence orresponding to the polypeptide of the invention, especially to a polypeptide as shown in Table II, application no. 1, colunms 5 or 8 or homologs thereof, or used in the process of the invention, whereas a “non- inventive protein or polypeptide” or “other polypeptide” refers to a polypeptide having an amino acid sequence corresponding to a protein which is not substantially homologous to a polypeptide of the invention, preferably which is not substantially homologous to a polypeptide or protein as shown in the respective line in Table II, application no. 1, column 5 or 8 or homologs thereof, e.g., a protein which does not confer the activity described herein and which is derived from the same or a different organism.

Within the fusion protein, the term “operatively linked” is intended to indicate that the polypeptide of the invention or a polypeptide used in the process of the invention and the “other polypeptide” or a part thereof are fused to each other so that both sequences fulfil the proposed function addicted to the sequence used. The “other polypeptide” can be fused to the N-terminus or C-terminus preferable to the C-terminus of the polypeptide of the invention or used in the process of the invention. For example, in one embodiment the fusion protein is a GST-LMRP fusion protein in which the sequences of the polypeptide of the invention or the polypeptide used in the process of the invention are fused to the C-terminus of the GST sequences. Such fusion proteins can facilitate the purification of recombinant polypeptides of the invention or a poylpeptide usefull in the process of the invention.

In another preferred embodiment, the fusion protein is a polypeptide of the invention or a polypeptide used in the process of the invention containing a heterologous signal sequence, preferably at its N-terminus. In certain host cells (e.g., mammalian host cells), expression and/or secretion of a polypeptide of the invention or a poylpeptide used in the process of the invention can be increased through use of a heterologous signal sequence. As already mentioned above, targeting sequences, are required for targeting the gene product into specific cell compartment (for a review, see Kermode, Crit. Rev. Plant Sci. 15 (4),285 (1996) and references cited therein), for example into the vacuole, the nucleus, all types of plastids, such as amyloplasts, chloroplasts, chromoplasts, the extracellular space, the mitochondria, the endoplasmic reticulum, elaioplasts, peroxisomes, glycosomes, and other compartments of cells or extracellular. Sequences, which must be mentioned in this context are, in particular, the signal-peptide- or transit-peptide-sequences which are known by the person skilled in the art. For example, plastid or mitochondrial-transit-peptide- sequences enable the targeting of the expression product into the plastids or the mitochondria, respectively, of a plant cell or the mitochondria of a microorganism. Targeting sequences are especially known for eukaryotic and to a lower extent for prokaryotic organisms and can advantageously be operable linked with the nucleic acid molecule of the present invention to achieve an expression in one of said compartments or extracellular.

Preferably, a chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. The fusion gene can be synthesized by conventional techniques including automated DNA synthesizers. Alternatively, PCR amplification of gene fragments can be carried out using anchor primers, which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons (1992)). Moreover, many expression vectors are commercially available that already encode a fusion moiety (e.g., a GST polypeptide). The nucleic acid molecule of the invention can be cloned into such an expression vector such that the fusion moiety is linked in-frame to the encoded protein.

Furthermore, folding simulations and computer redesign of structural motifs of the protein of the invention can be performed using appropriate computer programs (Olszewski, Proteins 25, 286 (1996); Hoffman, Comput. Appl. Biosci. 11, 675 (1995)). Computer modeling of protein folding can be used for the conformational and energetic analysis of detailed peptide and protein models (Monge, J. Mol. Biol. 247, 995 (1995); Renouf, Adv. Exp. Med. Biol. 376, 37 (1995)). The appropriate programs can be used for the identification of interactive sites the polypeptide of the invention or polypeptides used in the process of the invention and its substrates or binding factors or other interacting proteins by computer assistant searches for complementary peptide sequences (Fassina, Immunomethods 114 (1994)). This can be used to identify motifs and domains of the polypeptides according to the present invention or used in the process of the present invention. Further appropriate computer systems for the design of protein and peptides are described in the prior art, for example in Berry, Biochem. Soc. Trans. 22, 1033 (1994); Wodak, Ann. N.Y. Acad. Sci. 501, 1 (1987); Pabo, Biochemistry 25, 5987 (1986). The results obtained from the above-described computer analysis can be used for, e.g., the preparation of peptidomimetics of the protein of the invention or fragments thereof. Such pseudopeptide analogues of the natural amino acid sequence of the protein may very efficiently mimic the parent protein (Benkirane, J. Biol. Chem. 271, 33218 (1996)). For example, the replacement of Gly-Gly in a protein of the present invention or a fragment thereof by the easily available achiral omega-amino acid delta-amino valeric acid results in the substitution of —CH₂CONHCH₂— by —CH₂CH₂CH₂CH₂—, thereby providing a replacement of an amido group by an ethylen group, and a convenient strategy for constructing a peptidomimetic (Banerjee, Biopolymers 39, 769 (1996)).

Furthermore, a three-dimensional and/or crystallographic structure of the protein of the invention or used in the process of the present invention and the identification of motifs and/or interactive sites of the polypeptide of the invention or used in the process of the present invention and its substrates or binding factors can be used for the design of mutants with modulated binding or turnover activities. For example, the active center of the polypeptide of the present invention can be modelled and amino acids participating in the catalytic reaction can be modulated to increase or decrease the binding of the substrate to activate or improve the polypeptide. The identification of the active center, respective motifs and the amino acids involved in the catalytic reaction facilitates the screening for mutants having an increased activity.

Another embodiment of the invention relates to an antibody, which binds specifically to the polypeptide according to the invention or a portion thereof, i.e. specific fragments or epitopes of such a polypeptide.

The antibodies of the invention can be used to identify and isolate the polypeptide according to the invention in any organism, preferably microorganism or plants. These antibodies can be monoclonal antibodies, polyclonal antibodies or synthetic antibodies as well as fragments of antibodies, such as Fab, Fv or scFv fragments etc. Antibodies can be made by many well-known methods (see, e.g. Harlow and Lane, “Antibodies; A Laboratory Manual”, Cold Spring Harbor Laboratory, Cold Spring Harbor, N.Y., (1988)). Briefly, purified antigen can be injected into an animal in an amount and in intervals sufficient to elicit an immune response. Antibodies can either be purified directly, or spleen cells can be obtained from the animal. The cells can then be fused with an immortal cell line and screened for antibody secretion. The antibodies can be used to screen nucleic acid clone libraries for cells secreting the antigen. Those positive clones can then be sequenced. See, for example, Kelly et al., Bio/Technology 10, 163 (1992); Bebbington et al., Bio/Technology 10, 169 (1992).

As the nucleic acid molecules of the invention and/or to expression thereof is related to the synthesis of the respective fine chemical methionine its function as a probe extends to the detection of microorganisms, plant tissues, plants, plant variets, plant ecotypes or plant genera with varying, advantageously increased, capability or potential for synthesis of the respective fine chemical methionine. Therefore in one embodiment the present invention relates to a method for analyzing the capability or potential of a plant tissue, a plant, a plant variety or ecotype to produce the respective fine chemical methionine by using the respective antibody of the invention as a probe to detect the amount of the polypeptide encoded by said nucleic acid molecule of the invention in a non-human organism in comparision to another organism.

Therefore in one embodiment the invention relates to a method to probe a non-human organism for its capability for the production of the respective fine chemical comprising the steps of
a) analyzing the sequence and/or the expression of the respective nucleic acid molecule of the invention in least two non-human organisms which differ in the capability to produce the respective fine chemical;
b) associate thedifferent sequence and/or expression level of the nucleic acid molecules of the invention with their different capability to produce the respective fine chemical;
c) analyse the sequence and/or the expression of the nucleic acid molecule of the invention in at least a third non-human organism;
d) predict the capability of the at least third organism to produce the respective fine chemical based by comparing the sequence and/or expression of the nucleic acid of the invention to the sequences and/or expression levels of the nucleic acid molecule of the invention of the at least two organisms analyzed in step a.

The phrases “selectively binds” and “specifically binds” with the polypeptide refer to a binding reaction that is determinative of the presence of the polypeptide in a heterogeneous population of polypeptides and other biologics. Thus, under designated immunoassay conditions, the specified antibodies bound to a particular polypeptide do not bind in a significant amount to other polypeptides present in the sample. Selective binding of an antibody under such conditions may require an antibody that is selected for its specificity for a particular polypeptide. A variety of immunoassay formats may be used to select antibodies that selectively bind with a particular polypeptide. For example, solid-phase ELISA immunoassays are routinely used to select antibodies selectively immunoreactive with a polypeptide. See Harlow and Lane, “Antibodies, A Laboratory Manual,” Cold Spring Harbor Publications, New York, (1988), for a description of immunoassay formats and conditions that could be used to determine selective binding.

In some instances, it is desirable to prepare monoclonal antibodies from various hosts. A description of techniques for preparing such monoclonal antibodies may be found in Stites et al., eds., “Basic and Clinical Immunology,” (Lange Medical Publications, Los Altos, Calif., Fourth Edition) and references cited therein, and in Harlow and Lane, “Antibodies, A Laboratory Manual,” Cold Spring Harbor Publications, New York (1988).

Gene expression in non-human organism, like microorganism or plants, especially plants, is regulated by the interaction of protein transcription factors with specific nucleotide sequences within the regulatory region of a gene. One example of transcription factors are polypeptides that contain zinc finger (ZF) motifs. Each ZF module is approximately 30 amino acids long and folds around a zinc ion. The DNA recognition domain of a ZF protein is an α-helical structure that inserts into the major groove of the DNA double helix. The module contains three amino acids that bind to the DNA with each amino acid contacting a single base pair in the target DNA sequence. ZF motifs are arranged in a modular repeating fashion to form a set of fingers that recognize a contiguous DNA sequence. For example, a three-fingered ZF motif will recognize 9 by of DNA. Hundreds of proteins have been shown to contain ZF motifs with between 2 and 37 ZF modules in each protein (Isalan M. et al., Biochemistry 37 (35), 12026 (1998); Moore M. et al., Proc. Natl. Acad. Sci. USA 98 (4), 1432 (2001) and Moore M. et al., Proc. Natl. Acad. Sci. USA 98 (4), 1437 (2001); U.S Pat. No. 6,007,988 and U.S Pat. No. 6,013,453).

The regulatory region of a gene contains many short DNA sequences (cis-acting elements) that serve as recognition domains for transcription factors, including ZF proteins. Similar recognition domains in different genes allow the coordinate expression of several genes encoding enzymes in a metabolic pathway by common transcription factors. Variation in the recognition domains among members of a gene family facilitates differences in gene expression within the same gene family, for example, among tissues and stages of development and in response to environmental conditions.

Typical ZF proteins contain not only a DNA recognition domain but also a functional domain that enables the ZF protein to activate or repress transcription of a specific gene. Experimentally, an activation domain has been used to activate transcription of the target gene (U.S Pat. No. 5,789,538 and patent application WO 95/19431), but it is also possible to link a transcription repressor domain to the ZF and thereby inhibit transcription (WO 00/47754 and WO 01/002019). It has been reported that an enzymatic function such as nucleic acid cleavage can be linked to the ZF (WO 00/20622).

The invention provides a method that allows one skilled in the art to isolate the regulatory region of one or more FCRP encoding genes from the genome of a non-human organism, preferably a plant cell and to design zinc finger transcription factors linked to a functional domain that will interact with the regulatory region of the gene. The interaction of the zinc finger protein with the gene can be designed in such a manner as to alter expression of the gene and preferably thereby to confer the production or the increased production of the fine chemical as compared to a corresponding, e.g. non-transformed, wild type non-human organism, like a microorganism or a plant cell, plant or part thereof.

A further embodiment of the invention also relates to a method for the generation of a transgenic host or host cell, e.g. a eukaryotic or prokaryotic cell, preferably a transgenic microorganism, a transgenic plant cell or a transgenic plant tissue or a transgenic plant, which comprises introducing, into the plant cell, the plant or a part thereof , the nucleic acid molecule according to the invention, the nucleic acid construct according to the invention or the vector according to the invention or the expression cassette according to the invention.

A further embodiment of the invention also relates to a method for the stable generation of a host or host cell, prokaryotic or eukaryotic cell, preferably a transgenic microorganism such as a transgenic algae, a transgenic plant cell or a transgenic plant or a transgenic part thereof, which comprises introducing, into the plant cell, the plant or a part thereof, the nucleic acid construct according to the invention, the vector according to the invention, the expression cassette according to the invention, or the nucleic acid molecule according to the invention, whereby the introduction of the nucleic acid molecule, nucleic acid construct, expression cassette and/or vector is in such manner that the transformants are stable during the propagation of the host in respect of the introduced nucleic acid molecules, nucleic acid construct, expression cassette and/or vector.

A further embodiment of the invention also relates to a method for the transient generation of a host or host cell, prokaryotic or eukaryotic cell, preferably a transgenic microorganism such as a transgenic algae, a transgenic plant cell or a transgenic plant or a transgenic part thereof, which comprises introducing, into the plant cell, the plant or a part thereof, the nucleic acid construct according to the invention, the vector according to the invention, the expression cassette according to the invention or the nucleic acid molecule according to the invention, whereby the introduced nucleic acid molecule, nucleic acid construct, expression cassette and/or vector is not integrated into the genome of the host or host cell. Therefore the transformants are not stable during the propagation of the host in respect of the introduced nucleic acid molecules, nucleic acid construct, expressions cassette and/or vector.

In the process according to the invention, transgenic non-human organisms are also to be understood as meaning—if they take the form of plants—plant cells, plant tissues, plant organs such as root, shoot, stem, seed, flower, tuber or leaf, or intact plants which are grown for the production of the fine chemical.

Growing is to be understood as meaning for example culturing the transgenic plant cells, plant tissue or plant organs on or in a nutrient medium or the intact plant or part thereof on or in a substrate, for example in hydroponic culture, potting compost or on a field soil.

In a further advantageous embodiment of the process, the nucleic acid molecules can be expressed in single-celled plant cells (such as algae), see Falciatore et al., Marine Biotechnology 1 (3), 239 (1999), and references cited therein, and plant cells from higher plants (for example spermatophytes such as crops). Examples of plant expression vectors encompass those which are described in detail herein or in D. Becker, Plant Mol. Biol. 20,1195 (1992), M. W Bevan, [Nucl. Acids Res. 12, 8711(1984), and in “Vectors for Gene Transfer in Higher Plants” in: Transgenic Plants, Vol. 1, Engineering and Utilization, eds. S. D. Kung and R. Wu, Academic Press, 1993, pp. 15-38]. An overview of binary vectors and their use is also found in R. Hellens, Trends in Plant Science,5 (10), 446 (2000).

Vector DNA can be introduced into prokaryotic or eukaryotic cells via conventional transformation or transfection techniques. The terms “transformation” and “transfection” include conjugation and transduction and, as used in the present context, are intended to encompass a multiplicity of prior-art methods for introducing foreign nucleic acid molecules (for example DNA) into a host cell, including calcium phosphate coprecipitation or calcium chloride coprecipitation, DEAE-dextran-mediated transfection, PEG-mediated transfection, lipofection, natural competence, chemically mediated transfer, electroporation or particle bombardment. Suitable methods for the transformation or transfection of host cells, including plant cells, can be found in Sambrook et al. (Molecular Cloning: A Laboratory Manual., 2^{nd} Ed., Cold Spring Harbor Laboratory, Cold Spring Harbor Laboratory Press, Cold Spring Harbor, N.Y., 1989) and in other laboratory handbooks such as Methods in Molecular Biology, 1995, Vol. 44, Agrobacterium protocols, eds. Gartland and Davey, Humana Press, Totowa, N.J.

The above-described methods for the transformation and, if desired, regeneration of plants from plant cells, plant tissues or parts of a plant are exploited for transient or stable transformation of plants. Suitable methods are the transformation of protoplasts by polyethylene-glycol-induced DNA uptake, the biolistic method with the gene gun—known as the particle bombardment method—, electroporation, the incubation of dry embryos in DNA-containing solution, microinjection and the Agrobacterium-mediated gene transfer. The above-mentioned methods are described for example in B. Jenes, Techniques for Gene Transfer, in: Transgenic Plants, Vol. 1, Engineering and Utilization, edited by Kung S. D. and Wu R., Academic Press, pp. 128-143 (1993) and in Potrykus, Annu. Rev. Plant Physiol. Plant Molec. Biol. 42, 205 (1991). The construct to be expressed is preferably cloned into a vector, which is suitable for transforming Agrobacterium tumefaciens, for example pBin19 (Bevan, Nucl. Acids Res. 12, 8711 (1984)). Agrobacteria transformed with such a vector can then be used in the known manner for the transformation of plants, in particular crop plants, such as, for example, tobacco plants, for example by bathing scarified leaves or leaf segments in an agrobacterial solution and subsequently culturing them in suitable media. The transformation of plants with Agrobacterium tumefaciens is described for example by Hofgen and Willmitzer in Nucl. Acid Res. 16, 9877 (1988) or known from, inter alia, White F. F., “Vectors for Gene Transfer in Higher Plants” in Transgenic Plants, Vol. 1, Engineering and Utilization, edited by Kung S. D. and Wu R., Academic Press, 1993, pp. 15-38. Alternatively the construct to be expressed can be cloned into vectors suitable for plastid transformation, as for example described in WO 2004/029256, WO 20040/04445 or Dufourmantel et al., Plant Mol. Biol. 55, 479 (2004).

To select for the successful transfer of the nucleic acid molecule, vector or nucleic acid construct of the invention according to the invention into a host organism, it is advantageous to use marker genes as have already been described above in detail. It is known of the stable or transient integration of nucleic acids into plant cells that only a minority of the cells takes up the foreign DNA and, if desired, integrates it into its genome, depending on the expression vector used and the transfection technique used. To identify and select these integrants, a gene encoding for a selectable marker (as described above, for example resistance to antibiotics) is usually introduced into the host cells together with the gene of interest. Preferred selectable markers in plants comprise those, which confer resistance to an herbicide such as glyphosate or gluphosinate. Other suitable markers are, for example, markers, which encode genes involved in biosynthetic pathways of, for example, sugars or amino acids, such as β-galactosidase, ura3 or ilv2. Markers, which encode genes such as luciferase, gfp or other fluorescence genes, are likewise suitable. These markers and the aforementioned markers can be used in mutants in which these genes are not functional since, for example, they have been deleted by conventional methods. Furthermore, nucleic acid molecules, which encode a selectable marker, can be introduced into a host cell on the same vector as those, which encode the polypeptides of the invention or used in the process or else in a separate vector. Cells which have been transfected stably with the nucleic acid introduced can be identified for example by selection (for example, cells which have integrated the selectable marker survive whereas the other cells die).

Since the marker genes, as a rule specifically the gene for resistance to antibiotics and herbicides, are no longer required or are undesired in the transgenic host cell once the nucleic acids have been introduced successfully, the process according to the invention for introducing the nucleic acids advantageously employs techniques which enable the removal, or excision, of these marker genes. One such a method is what is known as cotransformation. The cotransformation method employs two vectors simultaneously for the transformation, one vector bearing the nucleic acid according to the invention and a second bearing the marker gene(s). A large proportion of transformants receives or, in the case of plants, comprises (up to 40% of the transformants and above), both vectors. In case of transformation with Agrobacteria, the transformants usually receive only a part of the vector, the sequence flanked by the T-DNA, which usually represents the expression cassette. The marker genes can subsequently be removed from the transformed plant by performing crosses. In another method, marker genes integrated into a transposon are used for the transformation together with desired nucleic acid (known as the Ac/Ds technology). The transformants can be crossed with a transposase resource or the transformants are transformed with a nucleic acid construct conferring expression of a transposase, transiently or stable. In some cases (approx. 10%), the transposon jumps out of the genome of the host cell once transformation has taken place successfully and is lost. In a further number of cases, the transposon jumps to a different location. In these cases, the marker gene must be eliminated by performing crosses. In microbiology, techniques were developed which make possible, or facilitate, the detection of such events. A further advantageous method relies on what are known as recombination systems, whose advantage is that elimination by crossing can be dispensed with. The best-known system of this type is what is known as the Cre/lox system. Cre1 is a recombinase, which removes the sequences located between the loxP sequences. If the marker gene is integrated between the loxP sequences, it is removed, once transformation has taken place successfully, by expression of the recombinase. Further recombination systems are the HIN/HIX, FLP/FRT and REP/STB system (Tribble et al., J. Biol. Chem., 275, 22255 (2000); Velmurugan et al., J. Cell Biol., 149, 553 (2000)). A site-specific integration into the plant genome of the nucleic acid sequences according to the invention is possible. Naturally, these methods can also be applied to microorganisms such as yeast, fungi or bacteria. Also methods for the production of marker-free plastid transformants using a transiently cointegrated selection gene have been described for example by Koop et al., Nature Biotechology, 22 (2), 225 (2004).

Agrobacteria transformed with an expression vector according to the invention may also be used in the manner known per se for the transformation of plants such as experimental plants like Arabidopsis or crop plants, such as, for example, cereals, maize, oats, rye, barley, wheat, soya, rice, cotton, sugarbeet, canola, sunflower, flax, hemp, potato, tobacco, tomato, carrot, bell peppers, oilseed rape, tapioca, cassava, arrow root, tagetes, alfalfa, lettuce and the various tree, nut, and grapevine species, in particular oil-containing crop plants such as soya, peanut, castor-oil plant, sunflower, maize, cotton, flax, oilseed rape, coconut, oil palm, safflower (Carthamus tinctorius) or cocoa beans, or in particular crop plants, like cereals, maize, oats, rye, barley, wheat, soya, rice, cotton, sugarbeet, canola, sunflower, potato or oilseed rape, for example by bathing scarified leaves or leaf segments in an agrobacterial solution and subsequently growing them in suitable media.

In addition to the transformation of somatic cells, which then have to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic (Feldman K. A. and Marks M. D., Mol. Gen. Genet. 208, 274 (1987), Feldmann K . in: Koncz C., Chua N.-H. and Shell J., eds., “Methods in Arabidopsis Research”, Word Scientific, Singapore, (1992) pp. 274-289). Alternative methods are based on the repeated removal of the influorescences and incubation of the excision site in the center of the rosette with transformed agrobacteria, whereby transformed seeds can likewise be obtained at a later point in time (Chang, Plant J., 5, 551 (1994), Katavic, Mol. Gen. Genet. 245, 363 (1994)). However, an especially effective method is the vacuum infiltration method with its modifications such as the “floral dip” method. In the case of vacuum infiltration of Arabidopsis, intact plants under reduced pressure are treated with an agrobacterial suspension (Bechthold N., C. R. Acad. Sci. Paris Life Sci, 316, 1194 (1993)), while in the case of the“floral dip” method the developing floral tissue is incubated briefly with a surfactant-treated agrobacterial suspension (Clough S. J., and Bent A. F., Plant J. 16, 735 (1998)). A certain proportion of transgenic seeds are harvested in both cases, and these seeds can be distinguished from non-transgenic seeds by growing under the above-described selective conditions. In addition the stable transformation of plastids is of advantages because plastids are inherited maternally in most crops reducing or eliminating the risk of transgene flow through pollen. The transformation of the chloroplast genome is generally achieved by a process, which has been schematically displayed in Klaus et al., (Nature Biotechnology 22 (2), 225 (2004)). Briefly the sequences to be transformed are cloned together with a selectable marker gene between flanking sequences homologous to the chloroplast genome. These homologous flanking sequences direct site specific integration into the plastome. Plastidal transformation has been described for many different plant species and an overview can be taken from Bock R., J. Mol. Biol. 312 (3), 425 (2001) or Maliga P., Trends Biotechnol. 21, 20 (2003). Further biotechnological progress has recently been reported in form of marker free plastid transformants, which can be produced by a transient cointegrated maker gene (Klaus et al., Nature Biotechnology 22 (2), 225 (2004)).

The genetically modified plant cells can be regenerated via all methods with which the skilled worker is familiar. Suitable methods can be found in the above-mentioned publications by Kung S. D. and Wu R., Potrykus or Höfgen and Willmitzer.

Accordingly, the present invention thus also relates to a plant cell comprising the nucleic acid construct according to the invention, the expression cassette according to the invention, the nucleic acid molecule according to the invention or the vector according to the invention, as well as a process to generate such a plant cell.

Accordingly the present invention relates to any cell transgenic for any nucleic acid characterized as part of the invention, e.g. conferring the increase of the respective fine chemical methionine in a cell or a non-human organism or a part thereof, e.g. the nucleic acid molecule of the invention, the nucleic acid construct of the invention, the vector of the invention, the expression cassette according to the invention, or a nucleic acid molecule encoding the polypeptide of the invention, e.g. encoding a polypeptide having an activity as the protein as shown in the respective line in Table II, application no. 1, column 3. Due to the above-mentioned activity the respective fine chemical methionine content in a cell or a non-human organism is increased. For example, due to modulation or manupulation, the cellular activity is increased, in a preferred embodiment in organelles such as plastids or mitochondria, e.g. due to an increased expression or specific activity or specific targeting of the subject matters of the invention in a cell or a non-human organism or a part thereof especially in organelles such as plastids or mitochondria, or in another embodiment in the cytosol. Transgenic for a polypeptide having a protein or a protein activity means herein that due to modulation or manipulation of the genome, the activity of protein as shown in the respective line in Table II, application no. 1, column 3 or a protein as shown in the respective line in Table II, application no. 1, column 3-like activity is increased in the cell or non-human organism or part thereof, especially in organelles such as plastids or mitochondria, or especuially in the cytosol. Examples are described above in context with the process of the invention.

“Transgenic”, for example regarding a nucleic acid molecule, a nucleic acid construct, an expression cassette or a vector comprising said nucleic acid molecule or a non-human organism transformed with said nucleic acid molecule, nucleic acid construct, expression casette or vector, refers to all those subjects originating by recombinant methods in which either
a) the nucleic acid sequence, or
b) a genetic control sequence linked operably to the nucleic acid sequence, for example a promoter, or
c) (a) and (b);
   are not located in their natural genetic environment or have been modified by recombinant methods, an example of a modification being a substitution, addition, deletion, inversion or insertion of one or more nucleotide residues. Natural genetic environment refers to the natural chromosomal locus in the organism of origin, or to the presence in a genomic library. In the case of a genomic library, the natural genetic environment of the nucleic acid sequence is preferably retained, at least in part. The environment flanks the nucleic acid sequence at least at one side and has a sequence of at least 50 bp, preferably at least 500 bp, especially preferably at least 1000 bp, very especially preferably at least 5000 bp, in length.

A naturally occurring expression cassette—for example the naturally occurring combination of the promoter of the gene encoding a protein as shown in the respective line in Table II, application no. 1, column 5 or 8, preferably the coding region thereof, or fragments or homologs thereof with the corresponding encoding gene—becomes a transgenic expression cassette when it is modified by non-natural, synthetic “artificial” methods such as, for example, mutagenesis. Such methods have been described (U.S. Pat. No. 5,565,350; WO 00/15815; also see above).

Further, the plant cell, plant or a part thereof can also be transformed such that further enzymes and proteins are (over)expressed which expression supports an increase of the fine chemical.

However, transgenic also means that the nucleic acids according to the invention are located at their natural position in the genome of a non-human organism, but that the sequence has been modified in comparison with the natural sequence and/or that the regulatory sequences of the natural sequences have been modified. Preferably, transgenic/recombinant is to be understood as meaning the transcription of the nucleic acids used in the process according to the invention occurs at a non-natural position in the genome, that is to say the expression of the nucleic acids is homologous or, preferably, heterologous. This expression can be performed with transiently transformed organism or with stably transformed organism.

Transgenic plants comprising the respective fine chemical(s) synthesized in the process according to the invention can be marketed directly without isolation of the fine chemical synthesized. In the process according to the invention, plants are understood as meaning all plant parts, plant organs such as leaf, stalk, root, tubers or seeds or propagation material or harvested material or the intact plant. In this context, the seed encompasses all parts of the seed such as the seed coats, epidermal cells, seed cells, endosperm or embryonic tissue. The fine chemical produced in the process according to the invention may, however, also be isolated from the plant. In case of methionine this can be in free form or bound to proteins. Fine chemical(s) produced by this process can be harvested by harvesting the non-human organisms either from the culture in which they grow or from the field. For example, this can be done via squeezing, grinding and/or extraction, salt precipitation and/or ion-exchange chromatography of the plant parts, preferably the plant seeds, plant fruits, plant tubers and the like.

In a further embodiment, the present invention relates to a process for the generation of a microorganism, comprising the introduction, into the microorganism or parts thereof, of the nucleic acid construct of the invention, or the expression cassette of the invention, or the vector of the invention or the nucleic acid molecule of the invention.

In another embodiment, the present invention relates also to a transgenic microorganism comprising the nucleic acid molecule of the invention, the nucleic acid construct of the invention, or the expression cassette of the invention, or the vector of the invention. Appropriate microorganisms have been described herein before under source organism, preferred are in particular aforementioned strains suitable for the production of fine chemicals.

In principle all microorganisms can be used as host organism especially the ones mentioned under source organism above. It is advantageous to use in the process of the invention transgenic microorganisms such as algae selected from the group of the families Bacillariophyceae, Charophyceae, Chlorophyceae, Chrysophyceae, Craspedophyceae, Euglenophyceae, Prymnesiophyceae, Phaeophyceae, Dinophyceae, Rhodophyceae, Xanthophyceae, Prasinophyceae and its described species and strains. Examples for such algae are the following species Isochrysis galbana, Chaetoceros gracilis, Chaetoceros calcitrans, Tetraselmis suecica, Thalassiosira pseudonana, Pavlova lutheri, lsochrysis sp., Skeletonema costatum, Chroomonas salina, Dunaliella tertiolecta, Chaetoceros simplex, Chaetoceros muelleri, Nannochloropsis sp., Cyclotella sp., Phaeodactylum tricornutum, Tetraselmis chui, Pavlova salina, Dicruteria sp., Tetraselmis levis, Dunaliella perva, Thalassiosira weissfloggii, Chlamydomonas sp., Chlorella vulgaris, Neochloris oleoabundans or Chlorella sp, which are only small overview.

The process of the invention is, when the host organisms are microorganisms, advantageously carried out at a temperature between 0° C. and 95° C., preferably between 10° C. and 85° C., particularly preferably between 15° C. and 75° C., very particularly preferably between 15° C. and 45° C. The pH is advantageously kept at between pH 4 and 12, preferably between pH 6 and 9, particularly preferably between pH 7 and 8, during this. The process of the invention can be operated batchwise, semibatchwise or continuously. A summary of known cultivation methods is to be found in the textbook by Chmiel (Bioprozeβtechnik 1. Einführung in die Bioverfahrenstechnik (Gustav Fischer Verlag, Stuttgart, 1991)) or in the textbook by Storhas (Bioreaktoren and periphere Einrichtungen (Vieweg Verlag, Braunschweig/Wiesbaden, 1994)). The culture medium to be used must meet the requirements of the respective strains in a suitable manner. Descriptions of culture media for various microorganisms are present in the handbook

“Manual of Methods for General Bacteriology” of the American Society for Bacteriology (Washington D. C., USA, 1981) and for algae in McLellan et al. (“Maintenance of algae and protozoa”, in “Maintenance of Microorganisms”, eds. Doyle A. and Kirsop B., London pp. 183-208 (1991)), Provasoli et al. (“Artificial media for freshwater algae: problems and suggestions”, in “The Ecology of Algae”, eds. Hartman R. T., Pymatunig Laboratory of Field Biology Special publication 2,

University of Pittsburgh, pp. 84-96 (1960)) or Starr R. C. (“Algal cultures-sources and methods of cultivation”, in “Photosynthesis Part A, Methods in Enzymology 23”, eds. San Pietro A., N.Y., pp 29-53 (1971)). These media, which can be employed according to the invention include, as described above, usually one or more carbon sources, nitrogen sources, inorganic salts, vitamins and/or trace elements. Preferred carbon sources are sugars such as mono-, di- or polysaccharides. Examples of very good carbon sources are glucose, fructose, mannose, galactose, ribose, sorbose, ribulose, lactose, maltose, sucrose, raffinose, starch or cellulose. Sugars can also be added to the media via complex compounds such as molasses, or other byproducts of sugar refining. It may also be advantageous to add mixtures of various carbon sources. Other possible carbon sources are oils and fats such as, for example, soybean oil, sunflower oil, peanut oil and/or coconut fat, fatty acids such as, for example, palmitic acid, stearic acid and/or linoleic acid, alcohols and/or polyalcohols such as, for example, glycerol, methanol and/or ethanol and/or organic acids such as, for example, acetic acid and/or lactic acid. Nitrogen sources are usually organic or inorganic nitrogen compounds or materials, which contain these compounds. Examples of nitrogen sources include ammonia in liquid or gaseous form or ammonium salts such as ammonium sulfate, ammonium chloride, ammonium phosphate, ammonium carbonate or ammonium nitrate, nitrates, urea, amino acids or complex nitrogen sources such as corn steep liquor, soybean meal, soybean protein, yeast extract, meat extract and others. The nitrogen sources may be used singly or as a mixture. Inorganic salt compounds, which may be present in the media include the chloride, phosphorus or sulfate salts of calcium, magnesium, sodium, cobalt, molybdenum, potassium, manganese, zinc, copper and iron. For the cultivation of algae the so called soilwater media are preferred. Such media are composed of soil extract, trace element solutions, filtered seawater, a nitrogen source and a buffer substance. Such culture media are well known by the skilled person and are available for example from culture collections such as the culture collection of algae (SAG) at the University of Gottingen, the Culture collection of algae in Coimbra, Portugal (ACOI) or the culture collection of algae (UTEX) in Texas, USA.

For preparing sulfur-containing amino acids, in particular methionin or cystein, it is possible to use as sulfur source inorganic sulfur-containing compounds such as, for example, sulfates, sulfites, dithionites, tetrathionates, thiosulfates, sulfides or else organic sulfur compounds such as mercaptans and thiols.

It is possible to use as phosphorus source phosphoric acid, potassium dihydrogenphosphate or dipotassium hydrogenphosphate or the corresponding sodium-containing salts. Chelating agents can be added to the medium in order to keep the metal ions in solution. Particularly suitable chelating agents include dihydroxyphenols such as catechol or protocatechuate, or organic acids such as citric acid. The fermentation media employed according to the invention for cultivating microorganisms normally also contain other growth factors such as vitamins or growth promoters, which include, for example, biotin, riboflavin, thiamine, folic acid, nicotinic acid, pantothenate and pyridoxine. All media components are sterilized either by heat (1.5 bar and 121° C. for 20 min) or by sterilizing filtration. The components can be sterilized either together or, if necessary, separately. All media components can be present at the start of the cultivation or optionally be added continuously or batchwise. The temperature of the culture is normally between 0° C. and 55° C., preferably at 10° C. to 30° C., and can be kept constant or changed during the experiment. The pH of the medium should be in the range from 3.5 to 8.5, preferably in the range between 5 to 7. The pH for the cultivation can be controlled during the cultivation by adding basic compounds such as sodium hydroxide, potassium hydroxide, ammonia or aqueous ammonia or acidic compounds such as phosphoric acid or sulfuric acid. Foaming can be controlled by employing antifoams such as, for example, fatty acid polyglycol esters. In addition the stability of plasmids can be maintained by adding to the medium suitable substances having a selective effect, for example antibiotics, if the plasmids carry the genetic information to overcome the selective effect. Aerobic conditions are maintained by introducing oxygen or oxygen-containing gas mixtures such as, for example, ambient air into the culture. The temperature of the culture is normally from 20° C. to 45° C. and preferably from 25° C. to 40° C. The culture is continued until formation of the desired product is at a maximum. This aim is normally achieved within 10 hours to 160 hours.

The fermentation broths obtained in this way, normally have a dry matter content of 7.5 to 25% by weight. The fermentation broth can be processed further. Depending on requirements, the biomass can be removed entirely or partly by separation methods, such as, for example, centrifugation, filtration, decantation or a combination of these methods, from the fermentation broth or left completely in it. The fermentation broth can then be thickened or concentrated by known methods, such as, for example, with the aid of a rotary evaporator, thin-film evaporator, falling film evaporator, by reverse osmosis or by nanofiltration. This concentrated fermentation broth can then be worked up by freeze-drying, spray drying, spray granulation or by other processes.

In another embodiment, the present invention relates to a method of producing a transgenic non-human oganism, especially a microorganism or a plant, with a FCRP coding nucleic acid, wherein expression of the nucleic acid(s) in the microorganism or a plant confers the production or the increased production of the fine chemical as compared to a wild type non-human organism comprising: (a) transforming a non-human cell with a vector comprising a FCRP encoding nucleic acid, and (b) generating from the non-human cell a transgenic non-human organism with production or enhanced production of the fine chemical.

The present invention relates also to a process according to the present invention whereby the produced fine chemical composition or the produced the fine chemical is isolated.

In this manner, more than 50% by weight, advantageously more than 60% by weight, preferably more than 70% by weight, especially preferably more than 80% by weight, very especially preferably more than 90% by weight, of the fine chemical produced in the process can be isolated. The resulting fine chemical can, if appropriate, subsequently be further purified, if desired mixed with other active ingredients such as vitamins, amino acids, carbohydrates, antibiotics and the like, and, if appropriate, formulated.

However, it is also possible to purify the fine chemical produced further with methods known by a person skilled in the art, like extraction, precipitation, crystallization etc.

For this purpose, the product-containing composition is for example subjected to a chromatography on a suitable resin, in which case the desired product or the impurities are retained wholly or partly on the chromatography resin. These chromatography steps can be repeated if necessary, using the same or different chromatography resins. The skilled worker is familiar with the choice of suitable chromatography resins and their most effective use. The purified product can be concentrated by filtration or ultrafiltration and stored at a temperature at which the stability of the product is a maximum.

The identity and purity of the isolated compound(s) can be determined by prior art techniques. These include gas chromatography (GC) high performance liquid chromatography (HPLC), spectroscopic methods, mass spectrometry (MS), staining methods, thin-layer chromatography, NIRS, enzyme assay or microbiological assays. These analytical methods are summarized for example in: Patek et al., Appl. Environ. Microbiol. 60, 133 (1994), Malakhova et al., Biotekhnologiya 11, 27 (1996); Schmidt et al., Bioprocess Engineer. 19, 67 (1998), Ullmann 's Encyclopedia of Industrial Chemistry (1996) Vol. A27, VCH, Weinheim, pp. 89-90, pp. 521-540, pp. 540-547, pp. 559-566, 575-581 and pp. 581-587, Michal G., “Biochemical Pathways: An Atlas of Biochemistry and Molecular Biology”, John Wiley and Sons (1999); Fallon A. et al. “Applications of HPLC in Biochemistry” in “Laboratory Techniques in Biochemistry and Molecular Biology”, Vol. 17 (1987).

The fine chemical obtained in the process are suitable as starting material for the synthesis of further products of value. For example, in case of amino acids they can be used in combination with each other or alone for the production of pharmaceuticals, foodstuffs, animal feeds or cosmetics. Accordingly, the present invention relates a method for the production of a pharmaceuticals, food stuff, animal feeds, nutrients or cosmetics comprising the steps of the process according to the invention, including the isolation of the fine chemical produced or a composition produced comprising the fine chemical, and, if desired, formulating the product with a pharmaceutical acceptable carrier or formulating the product in a form acceptable for an application in agriculture or formulating the product in a form acceptable for an application as food stuff, animal feed, nutrient or cosmetic. A further embodiment according to the invention is the use of the fine chemical produced in the process or of the transgenic non-human organisms in animal feeds, foodstuffs, medicines, food supplements, cosmetics or pharmaceuticals.

In yet another aspect, the invention also relates to harvestable parts and to propagation material of the transgenic plants according to the invention which either contain transgenic plant cells expressing a nucleic acid molecule according to the invention or which contains cells which show an increased cellular activity of the polypeptide of the invention, e.g. an increased expression level or higher activity of the described protein.

Harvestable parts can be in principle any useful parts of a plant, for example, flowers, pollen, seedlings, tubers, leaves, stems, fruit, seeds, roots etc. Propagation material includes, for example, seeds, fruits, cuttings, seedlings, tubers, rootstocks etc. Preferred are seeds, fruits, seedlings or tubers as harvestable or propagation material.

The invention furthermore relates to the use of the transgenic non-human organisms according to the invention and of the cells, cell cultures, parts—such as, for example, roots, leaves and the like as mentioned above in the case of transgenic plant organisms—derived from them, and to transgenic propagation material such as seeds or fruits and the like as mentioned above, for the production of foodstuffs or feeding stuffs, pharmaceuticals or fine chemicals.

Further in another embodiment, the present invention relates to the use of the nucleic acid molecule of the invention or used in the method of the invention alone or in combination with other genes of the respective fine chemical synthesis for example of the amino acid biosynthesis, the polypeptide of the invention or used in the method of the invention, the nucleic acid construct of the invention, the expression cassette of the invention, the vector of the invention, the plant or plant tissue or the host cell of the invention, for the production of plant resistant to a herbicide inhibiting eventually the production of the fine chemical.

Furthermore preferred is a method for the recombinant production of fine chemicals in non-human host organisms, wherein a non-human host organism is transformed with one of the above-described nucleic acid molecules or nucleic acid constructs, expression cassettes or vectors comprising one or more nucleic acid molecules, wherein said nucleic acid molecule participates in the biosynthesis of the desired fine chemical or encodes a polypeptide that catalyze the biosynthesis of the desired fine chemical, the transformed non-human host organism is cultured, and the desired fine chemical is isolated from the non-human organism and/or culture medium. In another embodiment s the additional production of further amino acids, tocopherols and tocotrienols and/or carotenoids or compositions comprising said compounds and/or of pharmaceuticals is preferred. The transformed non-human host organisms are cultured and the products are recovered from the non-human host organisms and/or the culture medium by methods known to the skilled worker or the non-human organism itself serves as food or feed or food or feed supplement. The production of pharmaceuticals such as, for example, antibodies or vaccines, is described by Hood E. E., ilkaJ. M. J, Curr. Opin. Biotechnol. 10 (4), 382 (1999), Ma J. K., Vine N. D., Curr. Top. Microbiol. Immunol. 236, 275 (1999).

In one embodiment, the present invention relates to a method for the identification of a gene product conferring the production of or an increase in the fine chemical production in a non-human cell, comprising the following steps:
(a) contacting, e.g. hybridising, a, some or all nucleic acid molecules of a sample, e.g. cells, tissues, plants or microorganisms or a nucleic acid library, which can contain a candidate gene encoding a gene product conferring the production of or an increase in the fine chemical after expression, with the nucleic acid molecule of the present invention as shown in the respective line in column 5 or 8 of Table I A or B of application no. 1, preferably the coding region thereof, or a fragment or homolog thereof;
(b) identifying the nucleic acid molecules, which hybridize under relaxed stringent conditions with the nucleic acid molecule of the present invention in particular to the nucleic acid molecule sequence shown in the respective line in Table I, application no. 1, columns 5 or 8, preferably the coding region thereof, of a fragment or a homolog thereof, preferably in Table I B, application no. 1, columns 5 or 8, preferably the coding region thereof, of a fragment or a homolog thereof, and, optionally, isolating the full length cDNA clone or complete genomic clone;
(c) introducing the candidate nucleic acid molecules or a fragment thereof in host cells, preferably in a plant cell or a microorganism, appropriate for producing the fine chemical;
(d) expressing the identified nucleic acid molecules in the host cells for which the production of or the increased production of the fine chemical is desired;
(e) assaying the the fine chemical level in the non-human host cells; and
(f) identifying the nucleic acid molecule and/or its gene product which expression confers the production of or an increase in the the fine chemical level in the non-human host cell after expression compared to the wild type.

Relaxed hybridisation conditions are: After standard hybridisation procedures washing steps can be performed at low to medium stringency conditions usually with washing conditions of 40° -55° C. and salt conditions between 2×SSC and 0.2×SSC with 0.1% SDS in comparison to stringent washing conditions as e.g. 60° -68° C. with 0.1% SDS. Further examples can be found in the references listed above for the stringend hybridization conditions. Usually washing steps are repeated with increasing stringency and length until a useful signal to noise ratio is detected and depend on many factors as the target, e.g. its purity, GC-content, size etc, the probe, e.g.its length, is it a RNA or a DNA probe, salt conditions, washing or hybridisation temperature, washing or hybridisation time etc.

In another embodiment, the present invention relates to a method for the identification of a gene product conferring the production of or an increase in the fine chemical production in a cell, comprising the following steps:
(a) identifiying a nucleic acid molecule of a non-human organism, which is at least 20%, 25%, 30%, 35%, 40%, 50%, 60%, 70%, 80%, 90%, 95%, 96%, 97%, 98%, 99%, 99.5% or more homolog to the nucleic acid molecule encoding a polypeptide comprising the polypeptide molecule as shown in the respective line in column 5 or 8 of Table II, application no. 1, or comprising a consensus sequence or a polypeptide motif as shown in the respective line in column 8 of Table IV, application no. 1, or being encoded by a nucleic acid molecule comprising a polynucleotide as shown in the respective line in column 5 or 8 of Table I, application no. 1, preferably the coding region thereof, or a homologue thereof as described herein, for example via homology search in a data bank;
(b) introducing the candidate nucleic acid molecule in non-human host cells, preferably in a plant cell or a microorganism, appropriate for producing the fine chemical;
(c) expressing the identified nucleic acid molecule in the non-human host cells;
(d) assaying the fine chemcial level in the non-human host cells; and
(e) identifying the nucleic acid molecule and its gene product which expression confers the production of or an increase in the the fine chemical level in the non-human host cell after expression compared to the wild type.

The nucleic acid molecules identified can then be used for the production of the fine chemical in the same way as the nucleic acid molecule of the present invention. Accordingly, in one embodiment, the present invention relates to a process for the production of the fine chemical, comprising (i) identifying a nucleic acid molecule according to aforementioned steps (a) to (f) or (a) to (e) and (ii) recovering the free or bound fine chemical from a non-human organism having an increased cellular activity of a polypeptide encoded by the nucleic acid molecule compared to a wild type and/or the respective culture medium.

Further, the nucleic acid molecules disclosed herein, in particular the nucleic acid molecules shown in the respective line in column 5 or 8 of Table I A or B of application no. 1, may be sufficiently homologous to the sequences of related species such that these nucleic acid molecules may serve as markers for the construction of a genomic map in related organism or for association mapping. Furthermore natural variation in the genomic regions corresponding to nucleic acids disclosed herein, in particular the nucleic acid molecule shown in the respective line in column 5 or 8 of Table I A or B, of application no. 1 or homologous thereof, may lead to variation in the activity of the proteins disclosed herein, in particular the proteins comprising polypeptides as shown in the respective lins in column 5 or 8 of Table II A or B, of application no. 1 or their homologous, or comprising the consensus sequence or the polypeptide motif as shown in the respective line in column 8 of Table IV, and in consequence in a natural variation of an increased fine chemical production.

In consequence natural variation eventually also exists in form of more active allelic variants leading already to a relative increase in the fine chemical. Different variants of the nucleic acid molecules disclosed herein, in particular the nucleic acid molecules comprising the nucleic acid molecules as shown in the respective line in column 5 or 8 of Table I A or B of application no. 1, preferably the coding region thereof, which corresponds to different levels of increase in fine chemical, e.g. different levels of increase in fine chemical, can be indentified and used for marker assisted breeding for an enhanced production of the fine chemical.

Natural variation may also exist in the regulatory regions, e.g. the promotors of the natural genes comprising the nucleic acid molecules as shown in the respective line column 5 or 8 of

Table I A or B of application no. 1, preferably the coding region thereof, which can similarily correspond to different levels of increase in the fine chemical, e.g. different levels of increase in the fine chemical can be identified and used for marker assisted breeding for an enhanced production of the fine chemical.

Accordingly, the present invention relates to a method for breeding plants for the production of the fine chemical, comprising
(a) selecting a first plant variety with an increased production of the fine chemical, e.g. based on an increased expression of a nucleic acid molecule of the invention as disclosed herein, in particular of a nucleic acid molecule comprising a nucleic acid molecule as shown in the respective line in column 5 or 8 of Table I A or B of application no. 1, preferably the coding region thereof, or fragments or homologs thereof, or a polypeptide comprising a polypeptide as shown in the respective line in column 5 or 8 of Table II A or B of application no. 1, or fragments or homologs thereof, or comprising a consensus sequence or a polypeptide motif as shown in the respective line in column 8 of Table IV, or a homolog thereof, as described herein;
(b) associating the level of increased production of the fine chemical, with the expression level or the genomic structure of a gene encoding said polypeptide or said nucleic acid molecule;
(c) crossing the first plant variety with a second plant variety, which significantly differs in its level of fine chemical; and
(d) identifying, which of the offspring varieties has got increased production of the fine chemical by the expression level of said polypeptide or nucleic acid molecule or the genomic structure of the gene(s) encoding said polypeptide or nucleic acid molecule of the invention.

In one embodiment, the expression level of the gene according to step (b) is increased.

In another embodiment, the present invention relates to a method for the identification of a compound stimulating production of the fine chemical in a non-human organism, especially a microorganism or a plant, comprising:
(a) contacting the microorganism or plant cells which express the polypeptide of the present invention or its mRNA with a candidate compound under appropriate conditions or in case of plant cells under cell cultivation conditions;
(b) assaying the production of or an increase in expression of said polypeptide or said mRNA;
(c) comparing the expression level to a standard response made in the absence of said candidate compound; whereby, the production of or an increased expression over the standard indicates that the compound is stimulating production of the fine chemical.

Furthermore, in one embodiment, the present invention relates to a process for the identification of a compound conferring the production of or an increase in the fine chemical production in a non-human organism, especially a plant or microorganism, comprising the steps:
(a) culturing a cell or a part of a plant or a microorganism or maintaining a plant expressing the polypeptide according to the invention or a nucleic acid molecule encoding said polypeptide and a readout system capable of interacting with the polypeptide under suitable conditions which permit the interaction of the polypeptide with said readout system in the presence of a compound or a sample comprising a plurality of compounds and capable of providing a detectable signal in response to the binding of a compound to said polypeptide under conditions which permit the expression of said readout system and the polypeptide of the present invention or used in the process of the invention; and
(b) identifying if the compound is an effective agonist by detecting the presence or absence or increase of a signal produced by said readout system.

The screen for a gene product or an agonist conferring the production of or an increase in the fine chemical can be performed by growth of a non-human organism for example a microorganism in the presence of growth reducing amounts of an inhibitor of the synthesis of the fine chemical. Better growth, e.g. higher dividing rate or high dry mass in comparison to the control, e.g. with a gene product or an agonist conferring the production of or an increase in the fine chemical, under such conditions would identify a gene or gene product or an agonist conferring the production of or an increase in fine chemical.

Said compound may be chemically synthesized or microbiologically produced and/or comprised in, for example, samples, e.g., cell extracts from, e.g., plants, non-human animals or microorganisms, e.g. pathogens. Furthermore, said compound(s) may be known in the art but hitherto not known to be capable of suppressing or activating the polypeptide of the present invention. The reaction mixture may be a cell free extract or may comprise a cell or tissue culture. Suitable set ups for the method of the invention are known to the person skilled in the art and are, for example, generally described in Alberts et al., “Molecular Biology of the Cell”, third edition (1994), in particular Chapter 17. The compounds may be, e.g., added to the reaction mixture, culture medium, injected into the cell or sprayed onto the plant.

If a sample containing a compound is identified in the method of the invention, then it is either possible to isolate the compound from the original sample identified as containing the compound capable of activating or conferring the production of or an increase in the fine chemical in a non-human organism or part thereof, or one can further subdivide the original sample, for example, if it consists of a plurality of different compounds, so as to reduce the number of different compounds per sample and repeat the method with the subdivisions of the original sample. Depending on the complexity of the samples, the steps described above can be performed several times, preferably until the sample identified according to the method of the invention only comprises a limited number of or only one compound(s). Preferably said sample comprises compounds of similar chemical and/or physical properties. Preferably, the compound identified according to the above-described method or a respective “active” derivative thereof is further formulated in a form suitable for the application in plant breeding or plant cell and tissue culture.

The compounds which can be tested and identified according to a method of the invention may be expression libraries, e.g., cDNA expression libraries, peptides, proteins, nucleic acids, antibodies, small organic compounds, hormones, peptidomimetics, PNAs or the like (Milner, Nature Medicine 1, 879 (1995); Hupp, Cell 83, 237 (1995); Gibbs, Cell 79, 193 (1994) and references cited supra). Said compounds can also be functional derivatives or analogues of known inhibitors or activators. Methods for the preparation of chemical derivatives and analogues are well known to those skilled in the art and are described in, for example, Beilstein, Handbook of Organic Chemistry, Springer edition New York Inc., 175 Fifth Avenue, New York, N.Y. 10010 U.S.A. and Organic Synthesis, Wiley, N.Y., USA. Furthermore, said derivatives and analogues can be tested for their effects according to methods known in the art. Furthermore, peptidomimetics and/or computer aided design of appropriate derivatives and analogues can be used, for example, according to the methods described above. The non-human cell or tissue that may be employed in the method of the invention preferably is a non-human host cell, plant cell or plant tissue of the invention described in the embodiments hereinbefore.

Thus, in a further embodiment the invention relates to a compound obtained or identified according to the method for identifiying an agonist of the invention said compound being an agonist of the polypeptide of the present invention or used in the process of the present invention.

Accordingly, in one embodiment, the present invention further relates to a compound identified by the method for identifying a compound of the present invention.

Said compound is, for example, a homolog of the polypeptide of the present invention. Homologs of the polypeptide of the present invention can be generated by mutagenesis, e.g., discrete point mutation or truncation of the polypeptide of the present invention. As used herein, the term “homolog” refers to a variant form of the protein, which acts as an agonist of the activity of the polypeptide of the present invention. An agonist of said protein can retain substantially the same, or a subset, of the biological activities of the polypeptide of the present invention. In particular, said agonist confers the increase of the expression level of the polypeptide of the present invention and/or the expression of said agonist in a non-human organisms or part thereof confers the increase of free and/or bound fine chemical in the non-human organism or part thereof.

In another embodiment, the invention relates to an antibody specifically recognizing the compound or agonist of the present invention.

The invention also relates to a diagnostic composition comprising at least one of the aforementioned nucleic acid molecules, expression cassettes, vectors, proteins, antibodies or compounds of the invention and optionally suitable means for detection.

The diagnostic composition of the present invention is suitable for the isolation of mRNA from a non-human cell and contacting the mRNA so obtained with a probe comprising a nucleic acid probe as described above under hybridizing conditions, detecting the presence of mRNA hybridized to the probe, and thereby detecting the expression of the protein in the non-human cell. Further methods of detecting the presence of a protein according to the present invention comprise immunotechniques well known in the art, for example enzyme linked immunoadsorbent assay. Furthermore, it is possible to use the nucleic acid molecules according to the invention as molecular markers or primers in plant breeding. Suitable means for detection are well known to a person skilled in the art, e.g. buffers and solutions for hybridization assays, e.g. the afore-mentioned solutions and buffers, and further means for Southern-, Western-, Northern- etc. -blots, as e.g. described in Sambrook et al. are known. In one embodiment diagnostic compositions contain PCR primers designed to specifically detect the presence or the expression level of the nucleic acid molecule to be expressed or to be expressed on an enhanced level in the process of the invention, e.g. of the nucleic acid molecule of the invention, or to descriminate between different variants or alleles of the nucleic acid molecule of the invention or nucleic acid molecules the activity of which is to be increased in the process of the invention.

In another embodiment, the present invention relates to a kit comprising the nucleic acid molecule, the vector, the host cell, the polypeptide, or the, viral nucleic acid molecule, antibody, plant cell, plant or part thereof, the harvestable part, the propagation material and/or the compound and/or agonist identified according to the method of the invention.

The compounds of the kit of the present invention may be packaged in containers such as vials, optionally with/in buffers and/or solvents. If appropriate, one or more of the components of said kits might be packaged in one and the same container or in different ones. Additionally or alternatively, one or more of said components might be adsorbed to a solid support, like a nitrocellulose filter, a glas plate, a chip, a nylon membrane or to the well of a micro titerplate. The kit can be used for any of the herein described methods and embodiments, e.g. for the production of the host cells, transgenic plants, pharmaceutical compositions, detection of homologous sequences, identification of agonists, as food or feed or as a supplement thereof or as supplement for the treating of plants, etc. Further, the kit can comprise instructions for the use of the kit for any of said embodiments. In one embodiment said kit comprises further a nucleic acid molecule encoding one or more of the aforementioned protein, and/or an antibody, a vector, a host cell, a plant cell, a plant or a part thereof. In another embodiment said kit comprises PCR primers to detect and increase the nucleic acid molecule to be increased in the process of the invention, e.g. of the nucleic acid molecule of the invention.

In a further embodiment, the present invention relates to a method for the production of a agricultural composition providing the nucleic acid molecule, the vector, or antibody of the present invention, the viral nucleic acid of the invention, or the polypeptide of the invention, the compound or agonist or comprising the steps of the method according to the invention for the identification of said compound or agonist; and formulating the nucleic acid molecule, the vector, or antibody of the present invention, the viral nucleic acid of the invention, the polypeptide of the invention or the agonist, or compound identified according to the methods or processes of the present invention or with use of the subject matters of the present invention in a form applicable as plant agricultural composition.

In another embodiment, the present invention relates to a method for the production of “the fine chemical”-production supporting plant culture composition comprising the steps of the method of the present invention; and formulating the compound identified in a form acceptable as agricultural composition.

Under “acceptable as agricultural composition” is understood, that such a composition optionally comprises auxiliaries which are customarily used for formulating crop protection agents.

In a further embodiment the nucleic acid molecule of the invention, the polypeptide of the invention, the nucleic acid construct of the invention, the expression cassette of the invention, the non-human organisms, the non-human host cell, the microorgansims, the plant, plant cell, or the part thereof, of the invention, the vector of the invention, the agonist identified with the method of the invention, the nucleic acid molecule identified with the method of the present invention, can be used for the production of the fine chemical or of the fine chemical and one or more other amino acids, in particular threonine, alanine, glutamin, glutamic acid, valine, aspargine, phenylalanine, leucine, proline , tryptophan tyrosine, isoleucine and arginine.

Accordingly, the nucleic acid molecule of the invention, or the nucleic acid molecule identified with the method of the present invention or the complement sequences thereof, the polypeptide of the invention, the nucleic acid construct of the invention, the expression cassette of the invention, the non-human organism, the non-human host cell, the microorgansim, the plant, plant cell, or the part thereof of the invention, the vector of the invention, the agonist identified with the method of the invention, the antibody of the present invention, can be used for the production of or the increase in the fine chemical in a non-human organism or part thereof, e.g. in a cell.

These and other embodiments are disclosed and encompassed by the description and examples of the present invention. Further literature concerning any one of the methods, uses and compounds to be employed in accordance with the present invention may be retrieved from public libraries, using for example electronic devices. For example the public database “Medline” may be utilized which is available on the Internet, for example under http://www.ncbi.nlm.nih.gov/PubMed/medline.html. Further databases and addresses, such as http://www.ncbi.nlm.nih.gov/, http://www.infobiogen.fr/, http://www.fmi.ch/biology/researchtools. html, hftp://www.tigr.org/, are known to the person skilled in the art and can also be obtained using, e.g., http://www.lycos.com.

The present invention is illustrated by the examples, which follow. The present examples illustrate the basic invention without being intended as limiting the subject of the invention. The content of all of the references, patent applications, patents and published patent applications cited in the present patent application is herewith incorporated by reference.

In a further embodiment the present invention is characterized by the features as disclosed in the following items:

Item 1. A process for the production of the fine chemical methionine, which comprises
(a) increasing or generating one or more activities selected from the group consisting of (DL)-glycerol-3-phosphatase, 2,3-di hydroxyphenylpropionate 1,2-dioxygenase, 2-oxoglutarate dehydrogenase E1 subunit, 49747384_SOYBEAN-protein, 5′-nucleotidase, acetolactate synthase small subunit, acetyl CoA carboxylase, adenosine kinase, arginine exporter protein, AO g09680-protein, At2g45420-protein, At4g32480-protein, ATP-binding component of a transport system, auxin response factor, b0012-protein, bl 003-protein, bl 522-protein, b2032-protein, b2345-protein, b2513-protein, b2673-protein, b3246-protein, b3346-protein, b3817-protein, b4029protein, beta-hydroxylase, bifunctional aspartokinase/homoserine dehydrogenase, calcium-dependent protein kinase, coproporphyrinogen III oxidase, cyclin, cystathionine gamma-synthase, cystathionine-lyase, dihydroxyacid dehydratase, DNA-binding protein, F-box protein, glutaredoxin, glutathione S-transferase, glycogenin, homocitrate synthase, homoserine dehydrogenase, hydrolase, L-serine dehydratase, major facilitator superfamily transporter protein, malic enzyme, membrane transport protein, monothiol glutaredoxin, monthiol glutaredoxin, oxidoreductase, peptidyl-prolyl cis-trans isomerase, phosphoadenosine phosphosulfate reductase, Photosystem I reaction center subunit XI, protein kinase, protein phosphatase, pyruvate kinase, Sec-independent protein translocase subunit, serine protease, serine/threonine-protein phosphatase, threonine aldolase, threonine dehydratase, threonine efflux protein, transcription factor, transport protein, uridine/cytidine kinase, valine-pyruvate transaminase, yhl013c-protein, yml084w-protein, yol160w-protein, yor392w-protein, and zinc finger protein, in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of methionine or a composition comprising methionine in said non-human organism or in the culture medium surrounding said non-human organism.

Item 2. A process for the production of the fine chemical methionine, which comprises
(A)
   (i) increasing or generating of the expression of; and/or
   (ii) increasing or generating the expression of an expression product of; and/or
   (iii) increasing or generating one or more activities of an expression product encoded by;
   at least one nucleic acid molecule comprising a nucleic acid molecule selected from the group consisting of:
(a) a nucleic acid molecule encoding the polypeptide shown in column 5 or 8 of Table II, preferably Table II B, application no. 1, or a homolog or a fragment thereof;
(b) a nucleic acid molecule shown in column 5 or 8 of Table I, preferably Table I B, application no. 1, or a homolog or a fragment thereof (preferably the coding region thereof);
(c) a nucleic acid molecule, which, as a result of the degeneracy of the genetic code, can be derived from a polypeptide sequence depicted in column 5 or 8 of Table II, preferably Table II B, application no. 1;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 1, or the coding region thereof;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 1;
(i) a nucleic acid molecule encoding a polypeptide having the activity represented by a respective protein as depicted in column 5 of Table II, application no. 1;
(j) a nucleic acid molecule which comprises a polynucleotide, which is obtained by amplifying a cDNA library or a genomic library using the primers as depicted in column 8 of Table III, application no. 1; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto; in a non-human organism or a part thereof, preferably a microorganism, a plant cell, a plant or a part thereof, as compared to a corresponding non-transformed wild type non-human organism or a part thereof; and
(B) growing the non-human organism or a part thereof under conditions which permit the production of methionine or a composition comprising methionine in said non-human organism or in the culture medium surrounding said non-human organism.

Item 3. A process of items 1 or 2, comprising of recovering methionine in its free or bound form.

Item 4. The process of any one of items 1 to 3, comprising the folowing steps:
(a) selecting a non-human organism or a part thereof expressing a polypeptide encoded by the nucleic acid molecule characterized in item 2;
(b) mutagenizing the selected non-human organism or the part thereof;
(c) comparing the activity or the expression level of said polypeptide in the mutagenized non-human organism or the part thereof with the activity or the expression of said polypeptide of the selected non-human organisms or the part thereof;
(d) selecting the mutated non-human organisms or parts thereof, which comprise an increased activity or expression level of said polypeptide compared to the selected non-human organism or the part thereof;
(e) optionally, growing and cultivating the non-human organisms or the parts thereof;
and
(f) recovering, and optionally isolating, the free or bound methionine produced by the selected mutated non-human organisms or parts thereof.

Item 5. The process of any one of items 1 to 4 wherein the activity of said protein or the expression of said nucleic acid molecule is increased or generated transiently or stably.

Item 6. An isolated nucleic acid molecule comprising a nucleic acid molecule selected from the group consisting of:
(a) a nucleic acid molecule encoding a polypeptide shown in Table II, application no. 1, column 5 or 8, preferably shown in Table II B, application no. 1, column 8, or a homolog or a fragment thereof;
(b) a nucleic acid molecule shown in Table I, application no. 1, column 5 or 8, preferably shown in Table I B, application no. 1, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 1, preferably in column 8 of Table II B, application no. 1;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 1, preferably shown in column 8 of Table I B, application no. 1, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 1,
(i) a nucleic acid molecule encoding a polypeptide having the activity represented by a respective protein as depicted in column 5 of Table II, application no. 1;
(j) a nucleic acid molecule comprising a polynucleotide which is obtained by amplifying a cDNA library or a genomic library using the primers in Table III, application no. 1, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (i),
or a nucleic acid molecule comprising a sequence which is complementary thereto.

Item 7. A nucleic acid construct which confers the expression of the nucleic acid molecule as defined in item 6, comprising one or more regulatory elements.

Item 8. An expression cassette comprising
(a) a promoter, preferably selected from the group consisting of Big35S, PCUbi, Super ans USP;
(b) a nucleic acid molecule as defined in item 6.

Item 9. A vector comprising the nucleic acid molecule as defined in item 6 or the nucleic acid construct as claimed in item 7 or the expression cassette as claimed in item 8.

Item 10. A host cell or a plant organelle, which has been transformed with the vector as claimed in item 9 or the nucleic acid molecule as defined in item 6 or the nucleic acid construct as claimed in item 7 or the expression cassette as claimed in item 8.

Item 11. The host cell of item 10, which is a plant cell or a microorganism.

Item 12. A process for producing a polypeptide, wherein the polypeptide is expressed in a host cell as claimed in item 10 or 11.

Item 13. A polypeptide produced by the process as claimed in item 12 or encoded by the nucleic acid molecule as defined in item 6.

Item 14. An antibody, which binds specifically to the polypeptide as claimed in item 13.

Item 15. A plant tissue, propagation material, harvested material or a plant or a part thereof comprising the host cell as claimed in item 10 or 11.

Item 16. A process for the identification of a compound conferring an increase in methionine production in a non-human organism, comprising the steps:
(a) culturing a plant cell or tissue or microorganism or maintaining a plant expressing the polypeptide encoded by the nucleic acid molecule as defined in item 6 conferring an increase in the amount of methionine in a non-human organism or a part thereof and a readout system capable of interacting with the polypeptide under suitable conditions which permit the interaction of the polypeptide with said readout system in the presence of a compound or a sample comprising a plurality of compounds and capable of providing a detectable signal in response to the binding of a compound to said polypeptide under conditions which permit the expression of said readout system and of the polypeptide encoded by the nucleic acid molecule of item 6 conferring an increase in the amount of methionine in a non-human organism or a part thereof;
(b) identifying if the compound is an effective agonist by detecting the presence or absence or increase of a signal produced by said readout system.

Item 17. A method for the production of an agricultural composition comprising the steps of the method of item 16 and formulating the compound identified in item 16 in a form acceptable for an application in agriculture.

Item 18. A composition comprising the nucleic acid molecule as defined in item 6, the polypeptide as claimed in item 13, the nucleic acid construct as claimed in item 7, the expression cassette as claimed in item 8, the vector as claimed in item 9, the compound as claimed in item 16, the antibody as claimed in item 14, and optionally an agricultural acceptable carrier.

Item 19. Use of the nucleic acid molecule as defined in item 6 for the identification of a nucleic acid molecule conferring an increase in methionine after expression.

Item 20. Cosmetic, pharmaceutical, food or feed composition comprising the nucleic acid molecule as defined in item 6, the polypeptide as claimed in item 13, the nucleic acid construct as claimed in item 7, the expression cassette as claimed in item 8, the vector as claimed in item 9, the antibody as claimed in item 14, the plant or a part thereof, or plant tissue as claimed in item 15, the harvested material or propagation material as claimed in item 15 or the host cell as claimed in items 10 or 11.

Item 21. Use of the nuclei acid molecule as defined in item 6, the polypeptide as claimed in item 13, the nucleic acid construct as claimed in item 7, the expression cassette as claimed in item 8, the vector as claimed in item 9, the plant or plant tissue as claimed in item 15, or the host cell as claimed in item 10 to 11 for the production of plant resistant to a herbicide inhibiting the production of methionine .

### EXAMPLES

Example 1

### Cloning of the Sequences as Shown in Table I, Column 5 or 8 in Escherichia Coli

The inventive sequences as shown in the respective line in Table I, column 5 or 8 were cloned into the plasmids pBR322 (Sutcliffe J. G., Proc. Natl. Acad. Sci. USA, 75, 3737 (1979)), pACYC 177 (Change and Cohen, J. Bacteriol. 134, 1141 (1978)), plasmids of the pBS series (pBSSK+, pBSSK- and others; Stratagene, LaJolla, USA) or cosmids such as SuperCos1 (Stratagene, LaJolla, USA) or Lorist6 (Gibson T. J., Rosenthal A. and Waterson R.H., Gene 53, 283 (1987) for expression in E. coli using known, well-established procedures (see, for example, J. Sambrook et al. “Molecular Cloning: A Laboratory Manual”. Cold Spring Harbor Laboratory Press (1989) or F. M. Ausubel et al., “Current Protocols in Molecular Biology”, John Wiley & Sons (1994)).

### Example 2

### DNA Sequencing and Computerized Functional Analysis

The DNA was sequenced by standard procedures, in particular the chain determination method, using AB1377 sequencers (see, for example, Fleischman R. D. et al., Science 269, 496 (1995)).

### Example 3

### In-Vivo and In-Vitro Mutagenesis

An in vivo mutagenesis of organisms such as Saccharomyces, Mortierella, Escherichia and others mentioned above, which are beneficial for the production of a fine chemical can be carried out by passing a plasmid DNA (or another vector DNA) containing the desired nucleic acid sequence or nucleic acid sequences through E. coli and other microorganisms (for example Bacillus spp. or yeasts such as Saccharomyces cerevisiae) which are not capable of maintaining the integrity of its genetic information. Usual mutator strains have mutations in the genes for the DNA repair system (for example mutHLS, mutD, mutT and the like; for comparison, see Rupp W. D., “DNA repair mechanisms in Escherichia coli and Salmonella”, pp. 2277-2294, ASM, Washington (1996)]. The skilled worker knows these strains. The use of these strains is illustrated for example in Greener A., and Callahan M., Strategies 7, 32 (1994).

In-vitro mutation methods such as increasing the spontaneous mutation rates by chemical or physical treatment are well known to the skilled person. Mutagens like 5-bromo-uracil, N-methyl-N-nitro-N-nitrosoguanidine (=NTG), ethyl methanesulfonate (=EMS), hydroxylamine and/or nitrous acid are widly used as chemical agents for random in-vitro mutagensis. The most common physical method for mutagensis is the treatment with UV irradiation. Another random mutagenesis technique is the error-prone PCR for introducing amino acid changes into proteins. Mutations are deliberately introduced during PCR through the use of error-prone DNA polymerases and special reaction conditions known to a person skilled in the art. For this method randomized DNA sequences are cloned into expression vectors and the resulting mutant libraries screened for altered or improved protein activity as described below.

Site-directed mutagensis method such as the introduction of desired mutations with an M13 or phagemid vector and short oligonucleotides primers is a well-known approach for site-directed mutagensis. The clou of this method involves cloning of the nucleic acid sequence of the invention into an M13 or phagemid vector, which permits recovery of single-stranded recombinant nucleic acid sequence. A mutagenic oligonucleotide primer is then designed whose sequence is perfectly complementary to nucleic acid sequence in the region to be mutated, but with a single difference: at the intended mutation site it bears a base that is complementary to the desired mutant nucleotide rather than the original. The mutagenic oligonucleotide is then allowed to prime new DNA synthesis to create a complementary full-length sequence containing the desired mutation. Another site-directed mutagensis method is the PCR mismatch primer mutagensis method also known to the skilled person. Dpnl site-directed mutagensis is a further known method as described for example in the Stratagene QuickchangeTM site-directed mutagenesis kit protocol. A huge number of other methods are also known and used in common practice.

Positive mutation events can be selected by screening the organisms for the production of the desired fine chemical.

### Example 4

### DNA Transfer between Escherichia Coli, Saccharomyces Cerevisiae and Mortierella Alpina

Shuttle vectors such as pYE22m, pPAC-ResQ, pClasper, pAUR224, pAMH10, pAML10, pAMT10, pAMU10, pGMH10, pGML10, pGMT10, pGMU10, pPGAL1, pPADH1, pTADH1, pTAex3, pNGA142, pHT3101 and derivatives thereof which allow the transfer of nucleic acid sequences between Escherichia coli, Saccharomyces cerevisiae and/or Mortierella alpina are available to the skilled worker. An easy method to isolate such shuttle vectors is disclosed by Soni R. and Murray J. A. H., Nucleic Acid Research, 20 (21), 5852 (1992). If necessary such shuttle vectors can be constructed easily using standard vectors for E. coli (Sambrook J. et al. “Molecular Cloning: A Laboratory Manual”, Cold Spring Harbor Laboratory Press (1989) or Ausubel F. M. et al. “Current Protocols in Molecular Biology”, John Wiley & Sons (1994)) and/or the aforementioned vectors, which have a replication origin for, and suitable marker from, Escherichia coli, Saccharomyces cerevisiae or Mortierella alpina added. Such replication origins are preferably taken from endogenous plasmids, which have been isolated from species used in the inventive process. Genes, which are used in particular as transformation markers for these species are genes for kanamycin resistance (such as those which originate from the Tn5 or Tn-903 transposon) or for chloramphenicol resistance (Winnacker E.L., “From Genes to Clones Introduction to Gene Technology”, VCH, Weinheim (1987)) or for other antibiotic resistance genes such as for G418, gentamycin, neomycin, hygromycin or tetracycline resistance.

Using standard methods, it is possible to clone a gene of interest into one of the above-described shuttle vectors and to introduce such hybrid vectors into the microorganism strains used in the inventive process. The transformation of Saccharomyces can be achieved for example by LiCI or sheroplast transformation (Bishop et al., Mol. Cell. Biol., 6, 3401 (1986), Sherman et al., “Methods in Yeasts in Genetics”, Cold Spring Harbor Lab. Cold Spring Harbor, N.Y. (1982), Agatep et al., Technical Tips Online 1998, 1:51: P01525 or Gietz et al., Methods Mol. Cell. Biol. 5,255 (1995)) or electroporation (Delorme E., Appl. Environ. Microbiol., 55 (9), 2242 (1989)).

If the transformed sequence(s) is/are to be integrated advantageously into the genome of the microorganism used in the inventive process for example into the yeast or fungi genome, standard techniques known to the skilled worker also exist for this purpose. Solinger et al. (Proc. Natl. Acad. Sci. U S A., 15, 8447 (2001)) and Freedman et al. (Genetics, 162, 15 (2002)) teaches a homolog recombination system dependent on rad 50, rad51, rad54 and rad59 in yeasts. Vectors using this system for homologous recombination are vectors derived from the

Ylp series. Plasmid vectors derived for example from the 2p-Vector are known by the skilled worker and used for the expression in yeasts. Other preferred vectors are for example pART1, pCHY21 or pEVP11 as they have been described by McLeod et al. (EMBO J., 6, 729 (1987)) and Hoffman et al. (Genes Dev. 5, 561 (1991)) or Russell et al. (J. Biol. Chem. 258, 143 (1983)). Other beneficial yeast vectors are plasmids of the REP, REP-X, pYZ or RIP series.

### Example 5

### Determining the Expression of the Mutant/Transgenic Protein

The observations of the acivity of a mutated, or transgenic, protein in a transformed host cell are based on the fact that the protein is expressed in a similar manner and in a similar quantity as the wild-type protein. A suitable method for determining the transcription quantity of the mutant, or transgenic, gene (a sign for the amount of mRNA which is available for the translation of the gene product) is to carry out a Northern blot (see, for example, Ausubel et al., “Current Protocols in Molecular Biology”, Wiley, New York (1988)), where a primer which is designed in such a way that it binds to the gene of interest is provided with a detectable marker (usually a radioactive or chemiluminescent marker) so that, when the total RNA of a culture of the organism is extracted, separated on a gel, applied to a stable matrix and incubated with this probe, the binding and quantity of the binding of the probe indicates the presence and also the amount of mRNA for this gene. Another method is a quantitative PCR. This information detects the extent to which the gene has been transcribed. Total cell RNA can be isolated for example from yeasts or E. coli by a variety of methods, which are known in the art, for example with the Ambion kit according to the instructions of the manufacturer or as described in Edgington et al., Promega Notes Magazine Number 41, 14 (1993).

Standard techniques, such as Western blot, may be employed to determine the presence or relative amount of protein translated from this mRNA (see, for example, Ausubel et al. “Current Protocols in Molecular Biology”, Wiley, New York (1988)). In this method, total cell proteins are extracted, separated by gel electrophoresis, transferred to a matrix such as nitrocellulose and incubated with a probe, such as an antibody, which binds specifically to the desired protein. This probe is usually provided directly or indirectly with a chemiluminescent or colorimetric marker, which can be detected readily. The presence and the observed amount of marker indicate the presence and the amount of the sought mutant protein in the cell. However, other methods are also known.

### Example 6

### Growth of Genetically Modified Organism: Media and Culture Conditions

Genetically modified Yeast, Mortierella or Escherichia coli are grown in synthetic or natural growth media known by the skilled worker. A number of different growth media for Yeast, Mortierella or Escherichia coli are well known and widely available. A method for clulturing Mortierella is disclosed by Jang et al. (Bot. Bull. Acad. Sin. 41, 41 (2000)). Mortierella can be grown at 20° C. in a culture medium containing: 10 g/l glucose, 5 g/l yeast extract at pH 6.5. Futhermore Jang et al. teaches a submerged basal medium containing 20 g/l soluble starch, 5 g/l Bacto yeast extract, 10 g/l KNO₃, 1 g/l KH₂PO₄, and 0.5 g/l MgSat x7H₂O, pH 6.5.

Said media, which can be used according to the invention usually consist of one or more carbon sources, nitrogen sources, inorganic salts, vitamins and trace elements. Preferred carbon sources are sugars such as mono-, di- or polysaccharides. Examples of very good carbon sources are glucose, fructose, mannose, galactose, ribose, sorbose, ribulose, lactose, maltose, sucrose, raffinose, starch or cellulose. Sugars may also be added to the media via complex compounds such as molasses or other by-products of sugar refining. It may also be advantageous to add mixtures of various carbon sources. Other possible carbon sources are alcohols and/or organic acids such as methanol, ethanol, acetic acid or lactic acid. Nitrogen sources are usually organic or inorganic nitrogen compounds or materials containing said compounds. Examples of nitrogen sources include ammonia gas, aqueous ammonia solutions or ammonium salts such as NH₄Cl, or (NH₄)₂SO₄NH₄OH, nitrates, urea, amino acids or complex nitrogen sources such as cornsteep liquor, soybean flour, soybean protein, yeast extract, meat extract and others. Mixtures of the above nitrogen sources may be used advantageously.

Inorganic salt compounds, which may be included in the media comprise the chloride, phosphorus or sulfate salts of calcium, magnesium, sodium, cobalt, molybdenum, potassium, manganese, zinc, copper and iron. Chelating agents may be added to the medium in order to keep the metal ions in solution. Particularly suitable chelating agents include dihydroxyphenols such as catechol or protocatechulate or organic acids such as citric acid. The media usually also contain other growth factors such as vitamins or growth promoters, which include, for example, biotin, riboflavin, thiamine, folic acid, nicotinic acid, panthothenate and pyridoxine. Growth factors and salts are frequently derived from complex media components such as yeast extract, molasses, cornsteep liquor and the like. The exact composition of the compounds used in the media depends heavily on the particular experiment and is decided upon individually for each specific case. Information on the optimization of media can be found in the textbook “Applied Microbiol. Physiology, A Practical Approach” (eds. Rhodes P.M., Stanbury P.F., IRL Press pp. 53-73 (1997)). Growth media can also be obtained from commercial suppliers, for example Standard 1 (Merck) or BHI (Brain heart infusion, DIFCO) and the like.

All media components are sterilized, either by heat (20 min at 1.5 bar and 121° C.) or by filter sterilization. The components may be sterilized either together or, if required, separately. All media components may be present at the start of the cultivation or added continuously or batchwise, as desired.

The culture conditions are defined separately for each experiment. The temperature is normally between 15° C. and 45° C. and may be kept constant or may be altered during the experiment. The pH of the medium should be in the range from 5 to 8.5, preferably around 7.0, and can be maintained by adding buffers to the media. An example of a buffer for this purpose is a potassium phosphate buffer. Synthetic buffers such as MOPS, HEPES, ACES and the like may be used as an alternative or simultaneously. The culture pH value may also be kept constant during the culture period by addition of, for example, NaOH or NH₄OH. If complex media components such as yeast extract are used, additional buffers are required less since many complex compounds have a high buffer capacity. When using a fermenter for the culture of microorganisms, the pH value can also be regulated using gaseous ammonia.

The incubation period is generally in a range of from several hours to several days. This time period is selected in such a way that the maximum amount of product accumulates in the fermentation broth. The growth experiments, which are disclosed can be carried out in a multiplicity of containers such as microtiter plates, glass tubes, glass flasks or glass or metal fermenters of various sizes. To screen a large number of clones, the microorganisms should be grown in microtiter plates, glass tubes or shake flasks, either using simple flasks or baffle flasks. 100 ml shake flasks filled with 10% (based on the volume) of the growth medium required are preferably used. The flasks should be shaken on an orbital shaker (amplitude 25 mm) at a rate ranging from 100 to 300 rpm. Evaporation losses can be reduced by maintaining a humid atmosphere; as an alternative, a mathematical correction should be carried out for the evaporation losses.

If genetically modified clones are examined, an unmodified control clone, or a control clone, which contains the basic plasmid without insertion, should also be included in the tests. If a transgenic sequence is expressed, a control clone should advantageously again be included in these tests. The medium is advantageously inoculated to an OD600 of 0.5 to 1.5 using cells which have been grown on agar plates, such as CM plates (10 g/l glucose, 2.5 g/l NaCl, 2 g/l urea, 10 g/l polypeptone, 5 g/l yeast extract, 5 g/l meat extract, 22 g/l agar, pH value 6.8 established with 2M NaOH), which have been incubated at 30° C. The media are inoculated for example by addition of a liquid preculture of seed organism such as E. coli or S. cerevisiae.

### Example 7

### Growth of Genetically Modified Algae: Media and Culture Conditions

### Growing Chlamydomonas:

Chlamydomonas reinhardtii is able to grow under various growth conditions. It is a unicellular algae. The cells of Chlamydomonas reinhardtii can be normally cultured autotrophically in the media mentioned below. Cells of Chlamydomonas reinhardtii can be cultivated at 25° C. under cool-white fluorescence light at 10,000 lux (120 μmol m⁻² s⁻¹ photosynthetically active radiation) as described by Ghirardi et al., Appl. Biochem. Biotechnol. 63, 141 (1997) or Semin et al., Plant. Physiol., 131, 1756 (2003).

### Chlamydomonas Growth Medium:

1 l growth medium is prepared by adding the following volumes of the stock solutions as mentioned below:
1 ml solution A, 5 ml solution B, 1 ml solution C, 1 ml solution D, 3 ml solution E, 3 ml solution F,
1 ml solution G, 1 ml solution H.
A) Trace elements solution:
1 g/l H₃BO₃
1 g/l ZnSO₄×7H₂O₂O
0.3 g/l MnSO₄×H₂O₂O
0.2 g/l CoCl₂×6H₂O₂O
0.2 g/l Na₂MoO₄×2H₂O₂O
0.04 g/l CuSO₄
   B) Na Citrate solution: 10% w/v Na citrate×2H₂O₂O
   C) Iron solution: 1% w/v FeCl₃×6H₂O₂O
   D) Calcium solution: 5.3% w/v CaCl₂×H₂O₂O
   E) Magnesium solution: 10% w/v MgSO₄×7H₂O₂O
   F) Ammonium solution: 10% w/v NH₄NO₃

G) Potassium solution: 10% w/v KH₂PO₄

H) Dipotassium solution 10% w/v K₂HPO₄

### Bristol's Soil Extract Medium:

Soil extract medium can generally be used for the growth of axenic and xenic algae cultures. The soil extract is prepared by adding a teaspoon of dry garden soil and a pinch of CaCO₃ to 200 ml distilled water and steaming said solution for approximately 2 h on two consecutive days. Afterwards the supernatant is decanted and added to the desired medium. To 940 ml bristol's solution 40 ml of soil extract medium is added.

### Bristol's Solution:

To 940 ml of distilled water, the following stock solutions are added:

| | | |
|---|---|---|
| 10 ml | NaNO₃ | (25 g/l) |
| 10 ml | CaCl₂ × 2 H₂O | (2.5 g/l) |
| 10 ml | MgSO₄ × 7 H₂O | (7.5 g/l) |
| 10 ml | K₂HPO₄ | (7.5 g/l) |
| 10 ml | KH₂PO₄ | (17.5 g/l) |
| 10 ml | NaCl | (2.5 g/l) |

Amplification and Cloning of DNA from Chlamydomonas Reinhardtii:

The DNA can be amplified by the polymerase chain reaction (PCR) from Chlamydomonas reinhardtii by the method of Howitt Crispin A. (BioTechniques 21, 32 (1996)).

Fine chemical Production in Chlamydomonas Reinhardtii:

The fine chemical production can be analysed as mentioned above. The proteins and nucleic acids can be analysed as mentioned below.

### Example 8

### In-Vitro Analysis of the Function of the Proteins Encoded by the Transformed Sequences

The determination of the activities and kinetic parameters of enzymes is well known in the art. Experiments for determining the activity of a specific modified enzyme must be adapted to the specific activity of the wild-enzyme type, which is well within the capabilities of the skilled worker. Overviews of enzymes in general and specific details regarding the structure, kinetics, principles, methods, applications and examples for the determination of many enzyme activities can be found for example in the following literature: Dixon M. and Webb E.C.: “Enzymes”, Longmans, London (1979); Fersht “Enzyme Structure and Mechanism”, Freeman, New York(1985); Walsh “Enzymatic Reaction Mechanisms” Freeman, San Francisco (1979); Price N. C., Stevens L., “Fundamentals of Enzymology” Oxford Univ. Press, Oxford (1982); Boyer P. D. (ed.), “The Enzymes”, 3rd ed. Academic Press, New York (1983); Bisswanger H., “Enzymkinetik”, 2^{nd}ed. VCH, Weinheim (1994); Bergmeyer H. U., Bergmeyer J., Grafβl M., (eds.) “Methods of Enzymatic Analysis”, 3^{rd}rd. Vol. I-XII, Verlag Chemie: Weinheim (1983-1986); and “Ullmann's Encyclopedia of Industrial Chemistry” Vol. A9, “Enzymes”, VCH, Weinheim, pp. 352-363 (1987).

### Example 9

### Analysis of the Effect of the Nucleic Acid Molecule on the Production of the Fine Chemical

The effect of the genetic modification in plants, fungi, algae, ciliates on the production of a fine chemical can be determined by growing the modified microorganisms for example Chlamydomonas reinhardtii under suitable conditions (such as those described above) and analyzing the medium and/or the cellular components for the increased production of the fine chemical. Such analytical techniques are well known to the skilled worker and encompass spectroscopy, thinlayer chromatography, various types of staining methods, enzymatic and microbiological methods and analytical chromatography such as high-performance liquid chromatography (see, for example, “Ullmann Encyclopedia of Industrial Chemistry”, Vol. A2, pp. 89-90 and pp. 443-613, VCH, Weinheim (1985); Fallon A. et al., “Applications of HPLC in Biochemistry” in “Laboratory Techniques in Biochemistry and Molecular Biology”, Vol. 17 (1987); Rehm et al. “Biotechnology”, Vol. 3, Chapter III: “Product recovery and purification”, pp. 469-714, VCH: Weinheim (1993); Belter P. A. et al. “Bioseparations: downstream processing for Biotechnology”, John Wiley and Sons (1988); Kennedy J. F. and Cabral J. M. S., “Recovery processes for biological Materials”, John Wiley and Sons(1992); Shaeiwitzand J. A., Henry J. D., “Biochemical Separations” in “Ullmann's Encyclopedia of Industrial Chemistry”, Vol. B3; chapter 11, pp. 1-27, VCH, Weinheim (1988); and Dechow, “Separation and purification techniques in biotechnology”, Noyes Publications (1989)).

In addition to the determination of the fermentation end product, other components of the metabolic pathways which are used for the production of the desired compound, such as intermediates and by-products, may also be analyzed in order to determine the total productivity of the organism, the yield and/or production efficiency of the compound. The analytical methods encompass determining the amounts of nutrients in the medium (for example sugars, hydrocarbons, nitrogen sources, phosphate and other ions), determining biomass composition and growth, analyzing the production of ordinary metabolites from biosynthetic pathways and measuring gases generated during the fermentation. Standard methods for these are described in “Applied Microbial Physiology” A Practical Approach, Rhodes P. M. and Stanbury P. F., eds. IRL Press, pp. 103-129; 131-163 and 165-192, and the references cited therein.

### Example 10

### Purification of a Fine Chemical e.g. an Amino Acid

The amino acid can be recovered from cells and/or from the supernatant of the above-described culture by a variety of methods known in the art. For example, the culture supernatant is recovered first. To this end, the cells are harvested from the culture by slow centrifugation. Cells can generally be disrupted or lysed by standard techniques such as mechanical force or sonication. The cell debris is removed by centrifugation and the supernatant fraction, if appropriate together with the culture supernatant, is used for the further purification of the amino acid. However, it is also possible to process the supernatant alone if the amino acid is present in the supernatant in sufficiently high a concentration. In this case, the amino acid, or the amino acid mixture, can be purified further for example via extraction and/or salt precipitation or via ion-exchange chromatography.

If required and desired, further chromatography steps with a suitable resin may follow, the amino acid, but not many contaminants in the sample, being retained on the chromatography resin or the contaminants, but not the sample with the product (amino acid), being retained on the resin. If necessary, these chromatography steps may be repeated, using identical or other chromatography resins. The skilled worker is familiar with the selection of suitable chromatography resin and the most effective use for a particular molecule to be purified. The purified product can be concentrated by filtration or ultrafiltration and stored at a temperature at which maximum product stability is ensured. Many purification methods, which are not limited to the above purification method are known in the art. They are described, for example, in Bailey J. E. and Ollis D. F., “Biochemical Engineering Fundamentals”, McGraw-Hill: New York (1986).

Identity and purity of the amino acid isolated can be determined by standard techniques of the art. They encompass high-performance liquid chromatography (HPLC), spectroscopic methods, mass spectrometry (MS), staining methods, thin-layer chromatography, NIRS, enzyme assay or microbiological assays. These analytical methods are compiled in: Patek et al., Appl. Environ. Microbiol. 60, 133 (1994); Malakhova et al., Biotekhnologiya 11, 27 (1996); and Schmidt et al., Bioprocess Engineer. 19, 67 (1998), “Ullmann's Encyclopedia of Industrial Chemistry” Vol. A27, VCH, Weinheim, pp. 89-90, pp. 521-540, pp. 540-547, pp. 559-566, 575-581 and pp. 581-587 (1996); Michel G., “Biochemical Pathways: An Atlas of Biochemistry and Molecular Biology”, John Wiley and Sons (1999); Fallon A. et al. “Applications of HPLC in Biochemistry in: Laboratory Techniques in Biochemistry and Molecular Biology”, Vol. 17 (1987).

### Example 11

### Cloning of the Sequences as Shown in Table I, Column 5 or 8 for the Expression in Plants

### Example 11a

### PCR Amplification of the Sequences

Unless otherwise specified, standard methods as described in Sambrook et al., Molecular Cloning: A laboratory manual, Cold Spring Harbor 1989, Cold Spring Harbor Laboratory Press are used.

The inventive sequences as shown in the respective line in Table I, column 5, prefareably the coding region thereof, were amplified by PCR as described in the protocol of the Pfu Ultra, Pfu Turbo or Herculase DNA polymerase (Stratagene). The composition for the protocol of the Pfu Ultra, Pfu Turbo or Herculase DNA polymerase was as follows: 1×PCR buffer (Stratagene), 0.2 mM of each dNTP, 100 ng genomic DNA of Saccharomyces cerevisiae (strain S288C; Research Genetics, Inc., now Invitrogen), Escherichia coli (strain MG1655; E. coli Genetic Stock Center), Synechocystis sp. (strain PCC6803), Azotobacter vinelandii (strain N.R. Smith,16), Thermus thermophilus (HB8) or 50 ng cDNA from various tissues and development stages of Arabidopsis thaliana (ecotype Columbia), Physcomitrella patens, Glycine max (variety Resnick), Brassica napus, Oryza sativa or Zea mays (variety B73, Mo17, A188), 50 μmol forward primer, 50 μmol reverse primer, with or without 1 M Betaine, 2.5 u Pfu Ultra, Pfu Turbo or Herculase DNA polymerase.

The amplification cycles were as follows:

1 cycle of 2-3 minutes at 94-95° C., then 25-36 cycles with 30-60 seconds at 94-95° C., 30-45 seconds at 50-60° C. and 210-480 seconds at 72° C., followed by 1 cycle of 5-10 minutes at 72° C., then 4-16° C.—preferably for Saccharomyces cerevisiae, Escherichia coli, Synechocystis sp., Azotobacter vinelandii, Thermus thermophilus.

In case of Arabidopsis thaliana, Brassica napus, Glycine max, Oryza sativa, Physcomitrella patens, Zea mays the amplification cycles were as follows:

1 cycle with 30 seconds at 94° C., 30 seconds at 61 ° C., 15 minutes at 72° C., then 2 cycles with 30 seconds at 94° C., 30 seconds at 60° C., 15 minutes at 72° C., then 3 cycles with 30 seconds at 94° C., 30 seconds at 59° C., 15 minutes at 72° C., then 4 cycles with 30 seconds at 94° C., 30 seconds at 58° C., 15 minutes at 72° C., then 25 cycles with 30 seconds at 94° C., 30 seconds at 57° C., 15 minutes at 72° C., then 1 cycle with 10 minutes at 72° C., then finally 4-16° C.

RNAs were generated with the RNeasy Plant Kit according to the standard protocol (Qiagen) and Superscript II Reverse Transkriptase was used to produce double stranded cDNA according to the standard protocol (Invitrogen).

ORF specific primer pairs for the genes to be expressed are shown in the respective line in Table III, column 8. The following adapter sequences were added to Saccharomyces cerevisiae ORF specific primers (see Table III) for cloning purposes:

SEQ ID NO: 20

| | | |
|---|---|---|
| i) | foward primer: | 5′-GGAATTCCAGCTGACCACC-3′ |

SEQ ID NO: 21

| | | |
|---|---|---|
| ii) | reverse primer: | 5′-GATCCCCGGGAATTGCCATG-3′ |

These adaptor sequences allow cloning of the ORF into the various vectors containing the Resgen adaptors, see table column E of Table c.

The following adapter sequences were added to Saccharomyces cerevisiae, Escherichia coli, Synechocystis sp., Azotobacter vinelandii, Thermus thermophilus, Arabidopsis thaliana, Brassica napus, Glycine max, Oryza sativa , Physcomitrella patens, or Zea mays ORF specific primers for cloning purposes:

| SEQ ID NO: 22 | | |
|---|---|---|
| iii) | forward primer: | 5′-TTGCTCTTCC-3′ |
| SEQ ID NO: 23 | | |
| iiii) | reverse primer: | 5′-TTGCTCTTCG-3′ |

The adaptor sequences allow cloning of the ORF into the various vectors containing the Colic adaptors, see table column E of Table c.

Therefore for amplification and cloning of Saccharomyces cerevisiae SEQ ID NO: 13376, a primer consisting of the adaptor sequence i) and the ORF specific sequence SEQ ID NO: 14166 and a second primer consisting of the adaptor sequence ii) and the ORF specific sequence SEQ ID NO: 14167 were used.

For amplification and cloning of Escherichia coli SEQ ID NO: 6810, a primer consisting of the adaptor sequence iii) and the ORF specific sequence SEQ ID NO: 6816 and a second primer consisting of the adaptor sequence iiii) and the ORF specific sequence SEQ ID NO: 6817 were used.

For amplification and cloning of Synechocystis sp. SEQ ID NO: 11211, a primer consisting of the adaptor sequence iii) and the ORF specific sequence SEQ ID NO: 11419 and a second primer consisting of the adaptor sequence iiii) and the ORF specific sequence SEQ ID NO: 11420 were used.

For amplification and cloning of Arabidopsis thaliana SEQ ID NO: 352, a primer consisting of the adaptor sequence iii) and the ORF specific sequence SEQ ID NO: 380 and a second primer consisting of the adaptor sequence iiii) and the ORF specific sequence SEQ ID NO: 381 were used.

For amplification and cloning of Azotobacter vinelandii SEQ ID NO: 5557, a primer consisting of the adaptor sequence iii) and the ORF specific sequence SEQ ID NO: 6033 and a second primer consisting of the adaptor sequence iiii) and the ORF specific sequence SEQ ID NO: 6034 were used.

For amplification and cloning of Glycine max SEQ ID NO: 69, a primer consisting of the adaptor sequence iii) and the ORF specific sequence SEQ ID NO: 343 and a second primer consisting of the adaptor sequence iiii) and the ORF specific sequence SEQ ID NO: 344 were used.

For amplification and cloning of Thermus thermophilus SEQ ID NO: 12698, a primer consisting of the adaptor sequence iii) and the ORF specific sequence SEQ ID NO: 12970 and a second primer consisting of the adaptor sequence iiii) and the ORF specific sequence SEQ ID NO: 12971 were used.

Following these examples every sequence disclosed in Table I, preferably column 5, especially the coding region thereof can be cloned by fusing the adaptor sequences to the respective specific primers sequences as disclosed in Table III, column 8 using the respective vectors shown in Table c.

Table c showing binary vectors used in Example 11

TABLE c
Overview of the different vectors used for cloning the ORFs; showing their
SEQ ID NOs (column A), their vector names (column B), the promoters they
contain for expression of the ORFs (column C), if present, the additional artificial
targeting sequence (column D), the adapter sequence (column E), the expression
type conferred by the promoter mentioned in column B (column F)
and the figure number (column G).

| | B | C | D | E | | |
|---|---|---|---|---|---|---|
| A | VectorNa- | Promoter | Target | Adapter | F | G |
| SegID | me | Name | Sequence | Sequence | Expression Type | Figure |
| 30 | pMTX0270 | Super | | Colic | non targeted constitu- | 6 |
| | p | | | | tive expression prefer- | |
| | | | | | entially in green tissues | |
| 31 | pMTX155 | Big35S | | Resgen | non targeted constitu- | 7 |
| | | | | | tive expression prefer- | |
| | | | | | entially in green tissues | |
| 32 | VC- | Super | FNR | Resgen | plastidic targeted consti- | 3 |
| | MME354- | | | | tutive expression pref- | |
| | 1QCZ | | | | erentially in green | |
| | | | | | tissues | |
| 34 | VC- | Super | IVD | Resgen | mitochondric targeted | 8 |
| | MME356- | | | | constitutive expression | |
| | 1QCZ | | | | preferentially in green | |
| | | | | | tissues | |
| 36 | VC- | USP | | Resgen | non targeted expression | 9 |
| | MME301- | | | | preferentially in seeds | |
| | 1QCZ | | | | | |
| 37 | pMTX461k | USP | FNR | Resgen | plastidic targeted ex- | 10 |
| | orrp | | | | pression preferentially | |
| | | | | | in seeds | |
| 39 | VC- | USP | IVD | Resgen | mitochondric targeted | 11 |
| | MME462- | | | | expression preferen- | |
| | 1QCZ | | | | tially in seeds | |
| 41 | VC- | Super | Colic | | non targeted constitu- | 1 |
| | MME220- | | | | tive expression prefer- | |
| | 1qcz | | | | entially in green tissues | |
| 42 | VC- | Super | FNR | Colic | plastidic targeted consti- | 4 |
| | MME432- | | | | tutive expression | |
| | 1qcz | | | | preferentially in green | |
| | | | | | tissues | |
| 44 | VC- | Super | IVD | Colic | mitochondric targeted | 12 |
| | MME431- | | | | constitutive expression | |
| | 1qcz | | | | preferentially in green | |
| | | | | | tissues | |
| 46 | VC- | PcUbi | | Colic | non targeted constitu- | 2 |
| | MME221- | | | | tive expression prefer- | |
| | 1qcz | | | | entially in green tissues | |
| 47 | pMTX447k | PcUbi | FNR | Colic | plastidic targeted consti- | 13 |
| | orr | | | | tutive expression | |
| | | | | | preferentially in green | |
| | | | | | tissues | |
| 49 | VC- | PcUbi | IVD | Colic | mitochondric targeted | 14 |
| | MME445- | | | | constitutive expression | |
| | 1qcz | | | | preferentially in green | |
| | | | | | tissues | |
| 51 | VC- | USP | | Colic | non targeted expression | 15 |
| | MME289- | | | | preferentially in seeds | |
| | 1qcz | | | | | |
| 52 | VC- | USP | FNR | Colic | plastidic targeted | 16 |
| | MME464- | | | | expression | |
| | 1qcz | | | | preferentially in seeds | |
| 54 | VC- | USP | IVD | Colic | mitochondric targeted | 17 |
| | MME465- | | | | expression in | |
| | 1qcz | | | | preferentially seeds | |
| 56 | VC- | Super | | Resgen | non targeted constitu- | 5 |
| | MME489- | | | | tive expression prefer- | |
| | 1QCZ | | | | entially in green tissues | |

In this Table c PcUbi refers to the PcUbi promoter (Kawalleck et al., Plant. Molecular Biology, 21, 673 (1993)) also being named p-PcUBI (e.g. in Table d), Super refers to the Super promoter (Ni et al,. Plant Journal 7, 661 (1995)) also being named p-Super (e.g. in Table d), Big35S refers to the enhanced 35S promoter (Comai et al., Plant Mol Biol 15, 373 (1990)) and USP to the USP promoter (Baumlein et al., Mol Gen Genet. 225(3), 459 (1991)) also being named p-USP in Table d.

### Example 11b

### Construction of Binary Vectors for Non-Targeted Expression of Proteins

“Non-targeted” expression in this context means, that no additional targeting sequences were added to the ORF to be expressed.

For non-targeted expression the binary vectors used for cloning were VC-MME220-1 qcz SEQ ID NO 41 (FIG. 1), VC-MME221-1qcz SEQ ID NO 46 (FIG. 2), and VC-MME489-1QCZ SEQ ID NO: 56 (FIG. 5), respectively. The binary vectors used for cloning the targeting sequence were VC-MME489-1QCZ SEQ ID NO: 56 (FIG. 5) and pMTX0270p SEQ ID NO 30 (FIG. 6), respectively. Other useful binary vectors are known to the skilled worker; an overview of binary vectors and their use can be found in Hellens R., Mullineaux P. and Klee H. (Trends in Plant Science, 5 (10), 446 (2000)). Such vectors have to be equally equipped with appropriate promoters and targeting sequences.

### Example 11c

### Construction of Binary Vectors for Plastidic-Targeted Expression of Proteins

For construction of a vector for plastid-targeted expression in preferential green tissues or preferential in seeds, the plastidic targeting sequence of the gene FNR from Spinacia oleracea was amplified. In order to amplify the targeting sequence, genomic DNA was extracted from leaves of 4 weeks old S. oleracea plants (DNeasy Plant Mini Kit, Qiagen, Hilden). The gDNA was used as the template for a PCR.

To enable cloning of the transit sequence into the vector VC-MME489-1QCZ and VC-MME301-1 QCZ an EcoRl restriction enzyme recognition sequence was added to both the forward and reverse primers, whereas for cloning in the vectors pMTX0270p, VC-MME220-1 qcz, VC-MME-221-1qcz and VC-MME289-1qcz a Pmel restriction enzyme recognition sequence was added to the forward primer and a Ncol site was added to the reverse primer.

FNR5EcoResgen
SEQ ID NO: 24
ATA GAA TTC GCA TAA ACT TAT CTT CAT AGT TGC C
FNR3EcoResgen
SEQ ID NO: 25
ATA GAA TTC AGA GGC GAT CTG GGC CCT
FNR5PmeColic
SEQ ID NO: 26
ATA GTT TAA ACG CAT AAA CTT ATC TTC ATA GTT GCC
FNR3NcoColic
SEQ ID NO: 27
ATA CCA TGG AAG AGC AAG AGG CGA TCT GGG CCC T

The resulting sequence SEQ ID NO: 28 amplified from genomic spinach DNA, comprised a 511TR (bp 1-165), and the coding region (bp 166-273 and 351-419). The coding sequence is interrupted by an intronic sequence from by 274 to by 350:

gcataaacttatcttcatagttgccactccaatttgctccttgaatct
cctccacccaatacataatccactcctccatcacccacttcactacta
aatcaaacttaactctgtttttctctctcctcctttcatttcttattc
ttccaatcatcgtactccgccatgaccaccgctgtcaccgccgctgtt
tctttcccctctaccaaaaccacctctctccgcccgaagctcctccgt
catttcccctgacaaaatcagctacaaaaaggtgattcccaatttcac
tgtgttttttattaataatttgttattttgatgatgagatgattaatt
tgggtgctgcaggttcctttgtactacaggaatgtatctgcaactggg
aaaatgggacccatcagggcccagatcgcctct

### (SEQ ID NO: 28)

The PCR fragment derived with the primers FNR5EcoResgen and FNR3EcoResgen was digested with EcoRl and ligated in the vectors VC-MME489-1QCZ and VC-MME301-1QCZ, that had also been digested with EcoRl. The correct orientation of the FNR targeting sequence was tested by sequencing. The vector generated in this ligation step were VC-MME354-1 QCZ and pMTX461 korrp, respectively.

The PCR fragment derived with the primers FNR5PmeColic and FNR3NcoColic was digested with Pmel and Ncol and ligated in the vectors pMTX0270p, VC-MME220-1qcz, VC-MME221-1qcz and VC-MME289-1qcz that had been digested with Smal and Ncol. The vectors generated in this ligation step were VC-MME432-1 qcz, VC-MME464-1 qcz and pMTX447korr, respectively.

For plastidic-targeted constitutive expression in preferentially green tissues an artifical promoter A(ocs)3AmasPmas promoter (Super promotor)) (Ni et al,. Plant Journal 7, 661 (1995), WO 95/14098) was used in context of the vector VC-MME354-1QCZ for ORFs from Saccharomyces cerevisiae as well as Arabidopsis thaliana and in context of the vector VC-MME432-1 qcz for ORFs from Escherichia coli as well as Arabidopsis thaliana, resulting in each case in an “inframe” fusion of the FNR targeting sequence with the ORFs.

For plastidic-targeted expression in preferentially seeds the USP promoter (Baumlein et al., Mol Gen Genet. 225(3):459-67 (1991)) was used in context of either the vector pMTX461 korrp for ORFs from Saccharomyces cerevisiae or in context of the vector VC-MME464-1 qcz for ORFs from Escherichia coli, resulting in each case in an “in-frame” fusion of the FNR targeting sequence with the ORFs.

For plastidic-targeted constitutive expression in preferentially green tissues and seeds the PcUbi promoter was used in context of the vector pMTX447korr for ORFs from Saccharomyces cerevisiae, Escherichia coli, Synechocystis sp., Azotobacter vinelandii, Thermus thermophilus, Arabidopsis thaliana, Brassica napus, Glycine max, Oryza sativa, Physcomitrella patens, or Zea mays, resulting in each case in an “in-frame” fusion of the FNR targeting sequence with the ORFs.

### Example 11d

### Construction of Binary Vectors for Mitochondric-Targeted Expression of Proteins

Amplification of the mitochondrial targeting sequence of the gene IVD from Arabidopsis thaliana and construction of vector for mitochondrial-targeted expression in preferential green tissues or preferential in seeds.

In order to amplify the targeting sequence of the IVD gene from A. thaliana, genomic DNA was extracted from leaves of A.thaliana plants (DNeasy Plant Mini Kit, Qiagen, Hilden). The gDNA was used as the template for a PCR.

To enable cloning of the transit sequence into the vectors VC-MME489-1 QCZ and VC-MME301-1QCZ an EcoRl restriction enzyme recognition sequence was added to both the forward and reverse primers, whereas for cloning in the vectors VC-MME220-1qcz, VC-MME221-1qcz and VC-MME289-1qcz a Pmel restriction enzyme recognition sequence was added to the forward primer and a Ncol site was added to the reverse primer.

IVD5EcoResgen
SEQ ID NO: 57
ATA GAA TTC ATG CAG AGG TTT TTC TCC GC
IVD3EcoResgen
SEQ ID NO: 58
ATA GAA TTC CGA AGA ACG AGA AGA GAA AG
IVD5PmeColic
SEQ ID NO: 59
ATA GTT TAA ACA TGC AGA GGT TTT TCT CCG C
IVD3NcoColic
SEQ ID NO: 60
ATA CCA TGG AAG AGC AAA GGA GAG ACG AAG AAC GAG

The resulting sequence (SEQ ID NO: 61) amplified from genomic A. thaliana DNA with IVD5EcoResgen and IVD3EcoResgen comprised 81 bp:

(SEQ ID NO: 61)
atgcagaggtttttctccgccagatcgattctcggttacgccgtcaa
gacgcggaggaggtctttctcttctcgttcttcg

The resulting sequence (SEQ ID NO: 62) amplified from genomic A. thaliana DNA with IVD5PmeColic and IVD3NcoColic comprised 89 bp:

(SEQ ID NO: 62)
atgcagaggtttttctccgccagatcgattctcggttacgccgtcaag
acgcggaggaggtctttctcttctcgttcttcgtctctcct

The PCR fragment derived with the primers IVD5EcoResgen and IVD3EcoResgen was digested with EcoRl and ligated in the vectors VC-MME489-1QCZ and VC-MME301-1QCZ that had also been digested with EcoRl. The correct orientation of the IVD targeting sequence was tested by sequencing. The vectors generated in this ligation step were VC-MME356-1 QCZ and VC-MME462-1 QCZ, respectively.

The PCR fragment derived with the primers IVD5PmeColic and IVD3NcoColic was digested with Pmel and Ncol and ligated in the vectors VC-MME220-1qcz, VC-MME221-1qcz and VC-MME289-1qcz that had been digested with Smal and Ncol. The vectors generated in this ligation step were VC-MME431-1qcz, VC-MME465-1qcz and VC-MME445-1qcz, respectively.

For mitochondrial-targeted constitutive expression in preferentially green tissues an artifical promoter A(ocs)3AmasPmas promoter (Super promotor) (Ni et al,. Plant Journal 7, 661 (1995), WO 95/14098) was used in context of the vector VC-MME356-1 QCZ for ORFs from Saccharomyces cerevisiae and in context of the vector VC-MME431-1qcz for ORFs from Escherichia coli, resulting in each case in an “in-frame” fusion between the IVD sequence and the respective ORFs.

For mitochondrial-targeted constitutive expression in preferentially seeds the USP promoter (Baumlein et al., Mol Gen Genet. 225 (3), 459 (1991)) was used in context of the vector VC-MME462-1 QCZ for ORFs from Saccharomyces cerevisiae and in context of the vector VCMME 465-1qcz for ORFs from Escherichia coli, resulting in each case in an “in-frame” fusion between the IVD sequence and the respective ORFs.

For mitochondrial-targeted constitutive expression in preferentially green tissues and seeds the PcUbi promoter was used in context of the vector VC-MME445-1 qcz for ORFs from Saccharomyces cerevisiae, Escherichia coli, Synechocystis sp., Azotobacter vinelandii, Thermus thermophilus, Arabidopsis thaliana, Brassica napus, Glycine max, Oryza sativa, Physcomitrella patens, or Zea mays, resulting in each case in an “in-frame” fusion between the IVD sequence and the respective ORFs.

Other useful binary vectors are known to the skilled worker; an overview of binary vectors and their use can be found in Hellens R., Mullineaux P. and Klee H., (Trends in Plant Science, 5 (10), 446 (2000)). Such vectors have to be equally equipped with appropriate promoters and targeting sequences.

### Example 11e

### Cloning of Inventive Sequences as Shown in Table I, Column 5 in the Different Expression Vectors

For cloning of for example SEQ ID NO: 6810 from Escherichia coli or any other ORF from Escherichia coli, Synechocystis sp., Azotobacter vinelandii, Thermus thermophilus, Arabidopsis thaliana or Glycine max the vector DNA was treated with the restriction enzymes Pacl and Ncol following the standard protocol (MBI Fermentas).

For cloning for example the ORFs of SEQ ID NO: 14171 from Saccharomyces cerevisiae or any other ORF from Saccharomyces cerevisiae into vectors containing the Resgen adaptor sequence the respective vector DNA was treated with the restriction enzyme Ncol.

For cloning of for example ORFs SEQ ID NO: 13376 from Saccharomyces cerevisiae or any other ORF from Saccharomyces cerevisiae into vectors containing the Colic adaptor sequence, the respective vector DNA was treated with the restriction enzymes Pacl and Ncol following the standard protocol (MBI Fermentas).

In all cases the reaction was stopped by inactivation at 70° C. for 20 minutes and purified over QlAquick or NucleoSpin Extract II columns following the standard protocol (Qiagen or Macherey-Nagel).

Then the PCR-product representing the amplified ORF with the respective adapter sequences and the vector DNA were treated with T4 DNA polymerase according to the standard protocol (MBI Fermentas) to produce single stranded overhangs with the parameters 1 unit T4 DNA polymerase at 37° C. for 2-10 minutes for the vector and 1-2 u T4 DNA polymerase at 15-17° C. for 10-60 minutes for the PCR product representing SEQ ID NO: 6810.

The reaction was stopped by addition of high-salt buffer and purified over QlAquick or NucleoSpin Extract II columns following the standard protocol (Qiagen or Macherey-Nagel).

According to this example the skilled person is able to clone all sequences disclosed in Table I, preferably column 5 or column 8, especially the coding region thereof.

Approximately 30-60 ng of prepared vector and a defined amount of prepared amplificate were mixed and hybridized at 65° C. for 15 minutes followed by 37° C. 0,1 ° C./1 seconds, followed by 37° C. 10 minutes, followed by 0,1 ° C./1 seconds, then 4-10 ° C.

The ligated constructs were transformed in the same reaction vessel by addition of competent E. coli cells (strain DHSalpha) and incubation for 20 minutes at 1° C. followed by a heat shock for 90 seconds at 42° C. and cooling to 1-4° C. Then, complete medium (SOC) was added and the mixture was incubated for 45 minutes at 37° C. The entire mixture was subsequently plated onto an agar plate with 0.05 mg/ml kanamycin and incubated overnight at 37° C.

The outcome of the cloning step was verified by amplification with the aid of primers which bind upstream and downstream of the integration site, thus allowing the amplification of the insertion. The amplifications were carried out as described in the protocol of Taq DNA polymerase (Gibco-BRL).

The amplification cycles were as follows:

1 cycle of 1-5 minutes at 94° C., followed by 35 cycles of in each case 15-60 seconds at 94° C., 15-60 seconds at 50-66° C. and 5-15 minutes at 72° C., followed by 1 cycle of 10 minutes at 72° C., then 4-16° C.

Several colonies were checked, but only one colony for which a PCR product of the expected size was detected was used in the following steps.

A portion of this positive colony was transferred into a reaction vessel filled with complete medium (LB) supplemented with kanamycin and incubated overnight at 37° C.

The plasmid preparation was carried out as specified in the Qiaprep or NucleoSpin Multi-96 Plus standard protocol (Qiagen or Macherey-Nagel).

### Example 11f

### Generation of Transgenic Plants which Express Sequences as Shown in Table I, Preferably Column 5

1-5 ng of the plasmid DNA isolated was transformed by electroporation or transformation into competent cells of Agrobacterium tumefaciens, of strain GV 3101 pMP90 (Koncz and Schell, Mol. Gen. Gent. 204, 383 (1986)). Thereafter, complete medium (YEP) was added and the mixture was transferred into a fresh reaction vessel for 3 hours at 28° C. Thereafter, all of the reaction mixture was plated onto YEP agar plates supplemented with the respective antibiotics, e.g. rifampicine (0.1 mg/ml), gentamycine (0.025 mg/ml and kanamycin (0.05 mg/ml) and incubated for 48 hours at 28° C.

The agrobacteria that contains the plasmid construct were then used for the transformation of plants.

A colony was picked from the agar plate with the aid of a pipette tip and taken up in 3 ml of liquid TB medium, which also contained suitable antibiotics as described above. The preculture was grown for 48 hours at 28° C. and 120 rpm.

400 ml of LB medium containing the same antibiotics as above were used for the main culture. The preculture was transferred into the main culture. It was grown for 18 hours at 28° C. and 120 rpm. After centrifugation at 4 000 rpm, the pellet was resuspended in infiltration medium (MS medium, 10% sucrose).

In order to grow the plants for the transformation, dishes (Piki Saat 80, green, provided with a screen bottom, 30×20×4.5 cm, from Wiesauplast, Kunststofftechnik, Germany) were half-filled with a GS 90 substrate (standard soil, Werkverband E.V., Germany). The dishes were watered overnight with 0.05% Proplant solution (Chimac-Apriphar, Belgium). A. thaliana C24 seeds (Nottingham Arabidopsis Stock Centre, UK; NASC Stock N906) were scattered over the dish, approximately 1 000 seeds per dish. The dishes were covered with a hood and placed in the stratification facility (8 h, 110 μmol m⁻² s⁻¹, 22° C.; 16 h, dark, 6° C.). After 5 days, the dishes were placed into the short-day controlled environment chamber (8 h, 130 μmol m⁻2 s⁻1, 22° C.; 16 h, dark, 20° C.), where they remained for approximately 10 days until the first true leaves had formed.

The seedlings were transferred into pots containing the same substrate (Teku pots, 7 cm, LC series, manufactured by Pöppelmann GmbH & Co, Germany). Five plants were pricked out into each pot. The pots were then returned into the short-day controlled environment chamber for the plant to continue growing.

After 10 days, the plants were transferred into the greenhouse cabinet (supplementary illumination, 16 h, 340 μmol m⁻² s⁻¹, 22° C.; 8 h, dark, 20° C.), where they were allowed to grow for further 17 days.

For the transformation, 6-week-old Arabidopsis plants, which had just started flowering were immersed for 10 seconds into the above-described agrobacterial suspension which had previously been treated with 10 μl Silwett L77 (Crompton S. A., Osi Specialties, Switzerland). The method in question is described by Clough J. C. and Bent A. F. (Plant J. 16, 735 (1998)).

The plants were subsequently placed for 18 hours into a humid chamber. Thereafter, the pots were returned to the greenhouse for the plants to continue growing. The plants remained in the greenhouse for another 10 weeks until the seeds were ready for harvesting.

Depending on the tolerance marker used for the selection of the transformed plants the harvested seeds were planted in the greenhouse and subjected to a spray selection or else first sterilized and then grown on agar plates supplemented with the respective selection agent. Since the vector contained the bar gene as the tolerance marker, plantlets were sprayed four times at an interval of 2 to 3 days with 0.02% BASTA® and transformed plants were allowed to set seeds.

The seeds of the transgenic A. thaliana plants were stored in the freezer (at −20° C.).

### Example 11g

### Plant Culture (Arabidopsis) for Bioanalytical Analyses

For the bioanalytical analyses of the transgenic plants, the latter were grown uniformly a specific culture facility. To this end the GS-90 substrate as the compost mixture was introduced into the potting machine (Laible System GmbH, Singen, Germany) and filled into the pots. Thereafter, 35 pots were combined in one dish and treated with Previcur. For the treatment, 25 ml of Previcur were taken up in 10 l of tap water. This amount was sufficient for the treatment of approximately 200 pots. The pots were placed into the Previcur solution and additionally irrigated overhead with tap water without Previcur. They were used within four days.

For the sowing, the seeds, which had been stored in the refrigerator (at −20° C.), were removed from the Eppendorf tubes with the aid of a toothpick and transferred into the pots with the compost. In total, approximately 5 to 12 seeds were distributed in the middle of the pot.

After the seeds had been sown, the dishes with the pots were covered with matching plastic hood and placed into the stratification chamber for 4 days in the dark at 4° C. The humidity was approximately 90%. After the stratification, the test plants were grown for 22 to 23 days at a 16 h-light, 8-h-dark rhythm at 20° C., an atmospheric humidity of 60% and a CO2 concentration of approximately 400 ppm. The light sources used were Powerstar HQI-T 250 W/D Daylight lamps from Osram, which generate a light resembling the solar color spectrum with a light intensity of approximately 220 μmol m⁻² s⁻¹.

Selection of transgenic plants was depending on the use resistance marker. In case of the bar gene as the resistance marker plantlets were sprayed three times at days 8-10 after sowing with 0.02% BASTA® (Glufosinate ammonium; Bayer CropScience, Germany).The resistant plants were thinned when they had reached the age of 14 days. The plants, which had grown best in the center of the pot were considered the target plants. All the remaining plants were removed carefully with the aid of metal tweezers and discarded.

During their growth, the plants received overhead irrigation with distilled water (onto the compost) and bottom irrigation into the placement grooves. Once the grown plants had reached the age of 23 days, they were harvested. In case their seeds are desired these had been harvested 10 to 12 weeks after sowing (once they are ripe).

### Example 11 h

### Metabolic Analysis of Transformed Plants

The modifications identified in accordance with the invention, in the content of above-described metabolites, were identified by the following procedure.

### a) Sampling and Storage of the Samples

Sampling was performed directly in the controlled-environment chamber. The plants, or respective parts thereof, like leafs, were cut using small laboratory scissors, rapidly weighed on laboratory scales, transferred into a pre-cooled extraction sleeve and placed into an aluminum rack cooled by liquid nitrogen. If required, the extraction sleeves can be stored in the freezer at −80° C. The time elapsing between cutting the plant/plant parts to freezing it in liquid nitrogen amounted to not more than 10 to 20 seconds.

### b) Lyophilization

During the experiment, care was taken that the plants either remained in the deep-frozen state (temperatures <−40° C.) or were freed from water by lyophilization until the first contact with solvents.

The aluminum rack with the plant samples in the extraction sleeves was placed into the precooled (−40° C.) lyophilization facility. The initial temperature during the main drying phase was −35° C. and the pressure was 0.120 mbar. During the drying phase, the parameters were altered following a pressure and temperature program. The final temperature after 12 hours was +30° C. and the final pressure was 0.001 to 0.004 mbar. After the vacuum pump and the refrigerating machine had been switched off, the system was flushed with air (dried via a drying tube) or argon.

### c) Extraction

### Extraction of Arabidopsis Green Tissue:

Immediately after the lyophilization apparatus had been flushed, the extraction sleeves with the lyophilized plant material were transferred into the 5 ml extraction cartridges of the ASE device (Accelerated Solvent Extractor ASE 200 with Solvent Controller and AutoASE software (DIONEX)).

The 24 sample positions of an ASE device (Accelerated Solvent Extractor ASE 200 with Solvent Controller and AutoASE software (DIONEX)) were filled with plant samples, including some samples for testing quality control.

The polar substances were extracted with approximately 10 ml of methanol/water (80/20, v/v) at T=70° C. and p=140 bar, 5 minutes heating-up phase, 1 minute static extraction. The more lipophilic substances were extracted with approximately 10 ml of methanol/dichloromethane (40/60, v/v) at T=70° C. and p=140 bar, 5 minute heating-up phase, 1 minute static extraction.

The two solvent mixtures were extracted into the same glass tubes (centrifuge tubes, 50 ml, equipped with screw cap and pierceable septum for the ASE (DIONEX)).

The solution was treated with internal standards: ribitol, L-glycine-2,2-d₂, L-alanine-2,3,3,3-d₄, methionine-methyl-d₃, and α-methylglucopyranoside and methyl nonadecanoate, methyl undecanoate, methyl tridecanoate, methyl pentadecanoate, methyl nonacosanoate.

The total extract was treated with 8 ml of water. The solid residue of the plant sample and the extraction sleeve were discarded.

The extract was shaken and then centrifuged for 5 to 10 minutes at at least 1400 g in order to accelerate phase separation. 1 ml of the supernatant methanol/water phase (“polar phase”, colorless) was removed for the further GC analysis, and 1 ml was removed for the LC analysis. The remainder of the methanol/water phase was discarded. 0.5 ml of the organic phase (“lipid phase”, dark green) was removed for the further GC analysis and 0.5 ml was removed for the LC analysis. All the portions removed were evaporated to dryness using the IR Dancer infrared vacuum evaporator (Hettich). The maximum temperature during the evaporation process did not exceed 40° C. Pressure in the apparatus was not less than 10 mbar.

### Extraction of Arabidopsis Seeds:

3 mg of Arabidopsis seeds are transferred into a 1.2-ml-stainless steel grinding jar and ground and extracted with a mixture of 770 μl methanol and 290 μl water. A solution containing commercially available standard substances (ribitol, L-glycine-2,2-d₂, L-alanine-2,3,3,3-d4, methionine-methyl-d₃, tryptophane-d₅, Arginine ¹³C₆¹⁵N₄, Pep3 (Boc-Ala-Gly-Gly-Gly-OH) and α-methylglucopyranoside) is added as internal standard. The extraction is performed using a stainless steel ball and a ball mill (Retsch MM 200, Retsch, Germany) operated at 30 Hz for 3 minutes. After centrifugation at 6000 rpm for 5 minutes 800 μl of the extraction solvent is transferred into a 2-ml-reaction tube (Eppendorf).

A solution of commercially available internal standard substances (Coenzyme Q1, Coenzyme Q2, Coenzyme Q4, and methyl nonadecanoate, undecanoic acid, tridecanoic acid, pentadecanoic acid, methyl nonacosanoate) is added to the residue as internal standard. For the extraction of lipophilic metabolites, 640 μl methylene chloride and 170 μl methanol are added and the sample is extracted in a ball mill operated at 30 Hz for 3 minutes. After centrifugation at 6000 rpm for 5 minutes 800 μl of the extraction solvent is transferred and combined with the extract of the first extraction step. After the addition of 400 μl of water and a centrifugation step to ensure proper separation of the organic and aqueous layer, two aliquots of 500 μl of the aqueous layer (polar phase) are taken for GC and LC analysis, respectively. Also two aliquots of 100 μl of the organic layer (lipid phase) are take for GC and LC analysis, respectively. All the portions removed were evaporated to dryness using the IR Dancer infrared vacuum evaporator (Hettich). The maximum temperature during the evaporation process did not exceed 40° C. Pressure in the apparatus was not less than 10 mbar.

### Extraction of Rice or Corn Seed Material:

20 rice or corn kernels are homogenized with a 50-ml-stainless steel grinding jar and ground with a stainless steel grinding ball using a ball mill (Retsch MM 200, Retsch, Germany) operated at 30 Hz for 3 minutes. The ground samples are lyophilized over night The initial temperature during the main drying phase was −35 ° C. and the pressure was 0.120 mbar. During the drying phase, the parameters were altered following a pressure and temperature program. The final temperature after 12 hours was +30° C. and the final pressure was 0.001 to 0.004 mbar. After the vacuum pump and the refrigerating machine had been switched off, the system was flushed with air (dried via a drying tube) or argon. 50 mg of the lyophilized kernel material are weighed into glass fibre extraction thimbles and extracted and further processed as described for the extraction of Arabidopsis green tissue.

### d) Processing the Lipid Phase for the LC/MS or LC/MS/MS Analysis

The lipid extract, which had been evaporated to dryness was taken up in mobile phase. The HPLC was run with gradient elution.

The polar extract, which had been evaporated to dryness was taken up in mobile phase. The HPLC was run with gradient elution.

### LC-MS Analysis:

The LC part was carried out on a commercially available LC/MS system from Agilent Technologies, USA. For polar extracts 10 μl are injected into the system at a flow rate of 200 μl/min. The separation column (Reversed Phase C18) was maintained at 15 ° C. during chromatography. For lipid extracts 5 μl are injected into the system at a flow rate of 200 μl/min. The separation column (Reversed Phase C18) was maintained at 30 ° C. HPLC was performed with gradient elution. The mass spectrometric analysis was performed on an Applied Biosystems API 4000 triple quadrupole instrument with turbo ion spray source. For polar extracts the instrument measured in negative ion mode in MRM-mode and fullscan mode from 100-1000 amu. For lipid extracts the instrument measured in positive ion mode in MRM-mode fullscan mode from 100-1000 amu. MS analysis is described in more detail in patent publication number WO 03/073464.

### e) Derivatization of the Lipid Phase for the GC/MS Analysis

### Derivatization of the Lipid Phase for the GC/MS Analysis:

For the transmethanolysis, a mixture of 140 μl of chloroform, 37 μl of hydrochloric acid (37% by weight HCl in water), 320 μl of methanol and 20 μl of toluene was added to the evaporated extract of the lipid phase. The vessel was sealed tightly and heated for 2 hours at 100° C., with shaking. The solution was subsequently evaporated to dryness. The residue was dried completely.

The methoximation of the carbonyl groups was carried out by reaction with methoxyamine hydrochloride (5 mg/ml in pyridine, 100 μl for 1.5 hours at 60° C.) in a tightly sealed vessel. 20 μl of a solution of odd-numbered, straight-chain fatty acids (solution of each 0.3 mg/ml of fatty acids from 7 to 25 carbon atoms and each 0.6 mg/ml of fatty acids with 27, 29 and 31 carbon atoms in 3/7 (v/v) pyridine/toluene) were added as time standards. Finally, the derivatization with 100 μl of N-methyl-N-(trimethylsilyl)-2,2,2-trifluoroacetamide (MSTFA) was carried out for 30 minutes at 60° C., again in the tightly sealed vessel. The final volume before injection into the GC was 220 μl.

### f) Derivatization of the Polar Phase for the GC/MS Analysis

The methoximation of the carbonyl groups was carried out by reaction with methoxyamine hydrochloride (5 mg/ml in pyridine, 50 μl for 1.5 hours at 60° C.) in a tightly sealed vessel. 10 μl of a solution of odd-numbered, straight-chain fatty acids (solution of each 0.3 mg/ml of fatty acids from 7 to 25 carbon atoms and each 0.6 mg/mL of fatty acids with 27, 29 and 31 carbon atoms in 3/7 (v/v) pyridine/toluene) were added as time standards. Finally, the derivatization with 50 μl of N-methyl-N-(trimethylsilyl)-2,2,2-trifluoroacetamide (MSTFA) was carried out for 30 minutes at 60° C., again in the tightly sealed vessel. The final volume before injection into the GC was 110 μl.

### g) GC-MS Analysis

The GC-MS systems consisted of an Agilent 6890 GC coupled to an Agilent 5973 MSD. The autosamplers were CompiPal or GCPal from CTC. For the analysis usual commercial capillary separation columns (30 m×0.25 mm×0.25 pm) with different poly-methyl-siloxane stationary phases containing 0% up to 35% of aromatic moieties, depending on the analysed sample materials and fractions from the phase separation step, were used (for example: DB-1 ms, HP-5ms, DB-XLB, DB-35ms, Agilent Technologies). Up to 1 μl of the final volume was injected splitless and the oven temperature program was started at 70 ° C. and ended at 340 ° C. with different heating rates depending on the sample material and fraction from the phase separation step in order to achieve a sufficient chromatographic separation and number of scans within each analyte peak. Usual GC-MS standard conditions, for example constant flow with nominal 1 to 1.7 ml/min. and helium as the mobile phase gas were used. Ionisation was done by electron impact with 70 eV, scanning within a m/z range from 15 to 600 with scan rates from 2.5 to 3 scans/sec and standard tune conditions.

### h) Analysis of the Various Plant Samples

The samples were measured in individual series of 20 to 21 plant or seed samples each (also referred to as sequences), each sequence containing at least 5 wild-type plants or seeds as controls. Seed samples were from individual plants. The peak area of each analyte was divided by the peak area of the respective internal standard. The data were standardized for the fresh weight established for the plant or seed sample, respectively. The values calculated thus were related to the wild-type control group by being divided by the mean of the corresponding data of the wild-type control group of the same sequence. The values obtained were referred to as ratio_by_weight, they are comparable between sequences and indicate how much the analyte concentration in the transgenic plant sample differs in relation to the wild-type control. Appropriate controls were done before to proof that the vector and transformation procedure itself has no significant influence on the metabolic composition of the plants. Therefore the described changes in comparison with wild-types were caused by the introduced genes. At least 3-5 independent lines were analyzed in two independent experiments for each construct.

As an alternative, the amino acids can be detected advantageously via HPLC separation in ethanolic extract as described by Geigenberger et al. (Plant Cell & Environ, 19, 43 (1996)).

The results of the different plant analyses can be seen from the Table d.

### Table d Showing Results of Plant Analyses

TABLE d

| SeqID | Target | Locus | Metabolite | Source | Promotor | Method | Min | Max |
|---|---|---|---|---|---|---|---|---|
| 69 | non- | 49747384_ | methionine | ARA_LEAF | p-PcUBI | GC | 41 | 74 |
| | targeted | SOYBEAN | | | | | | |
| 352 | non- | At1g09680 | methionine | ARA_LEAF | p-PcUBI | LC | 41 | 60 |
| | targeted | | | | | | | |
| 385 | non- | At1g14490 | methionine | ARA_LEAF | p-PcUBI | LC | 38 | 52 |
| | targeted | | | | | | | |
| 627 | non- | At1g31230 | methionine | ARA_LEAF | Big35S | GC | 60 | 306 |
| | targeted | | | | | | | |
| 812 | non- | At1g48040 | methionine | ARA_LEAF | p-PcUBI | GC | 47 | 49 |
| | targeted | | | | | | | |
| 1061 | non- | At1g68320 | methionine | ARA_LEAF | p-PcUBI | GC | 38 | 47 |
| | targeted | | | | | | | |
| 1298 | non- | At2g25070 | methionine | ARA_LEAF | p-PcUBI | LC | 42 | 89 |
| | targeted | | | | | | | |
| 1623 | non- | At2g45420 | methionine | ARA_LEAF | p-PcUBI | LC | 32 | 37 |
| | targeted | | | | | | | |
| 1696 | non- | At3g09820 | methionine | ARA_LEAF | p-PcUBI | LC | 53 | 53 |
| | targeted | | | | | | | |
| 1815 | non- | At3g23000 | methionine | ARA_LEAF | p-PcUBI | GC | 43 | 67 |
| | targeted | | | | | | | |
| 2367 | non- | At3g61830 | methionine | ARA_LEAF | p-PcUBI | GC | 36 | 147 |
| | targeted | | | | | | | |
| 2573 | non- | At3g62930 | methionine | ARA_LEAF | p-PcUBI | GC | 37 | 63 |
| | targeted | | | | | | | |
| 2935 | non- | At3g62950 | methionine | ARA_LEAF | p-PcUBI | GC | 37 | 77 |
| | targeted | | | | | | | |
| 3279 | non- | At4g15670 | methionine | ARA_LEAF | p-PcUBI | LC | 37 | 52 |
| | targeted | | | | | | | |
| 3654 | non- | At4g15700 | methionine | ARA_LEAF | p-PcUBI | GC | 43 | 84 |
| | targeted | | | | | | | |
| 4040 | non- | At4g32480 | methionine | ARA_LEAF | p-PcUBI | LC | 44 | 184 |
| | targeted | | | | | | | |
| 4102 | non- | At4g33040 | methionine | ARA_LEAF | p-PcUBI | GC | 34 | 54 |
| | targeted | | | | | | | |
| 4348 | non- | At4g35310 | methionine | ARA_LEAF | p-PcUBI | GC | 62 | 83 |
| | targeted | | | | | | | |
| 4904 | non- | At5g18600 | methionine | ARA_LEAF | p-PcUBI | GC | 40 | 75 |
| | targeted | | | | | | | |
| 5318 | non- | At5g57050 | methionine | ARA_LEAF | p-PcUBI | GC | 36 | 45 |
| | targeted | | | | | | | |
| 5493 | non- | At5g64920 | methionine | ARA_LEAF | p-PcUBI | GC | 34 | 37 |
| | targeted | | | | | | | |
| 5557 | non- | AvinDRAFT_1495 | methionine | ARA_LEAF | p-PcUBI | GC | 38 | 42 |
| | targeted | | | | | | | |
| 6040 | non- | AvinDRAFT_2091 | methionine | ARA_LEAF | p-PcUBI | GC | 37 | 39 |
| | targeted | | | | | | | |
| 6075 | non- | AvinDRAFT_3028 | methionine | ARA_LEAF | p-PcUBI | GC | 39 | 55 |
| | targeted | | | | | | | |
| 6268 | non- | AvinDRAFT_3546 | methionine | ARA_LEAF | p-PcUBI | LC | 58 | 172 |
| | targeted | | | | | | | |
| 6510 | non- | AvinDRAFT_5103 | methionine | ARA_LEAF | p-PcUBI | GC | 280 | 288 |
| | targeted | | | | | | | |
| 6674 | non- | AvinDRAFT_6075 | methionine | ARA_LEAF | p-PcUBI | LC | 37 | 46 |
| | targeted | | | | | | | |
| 6810 | non- | B0012 | methionine | ARA_LEAF | p-Super | LC | 40 | 66 |
| | targeted | | | | | | | |
| 6818 | non- | B0078 | methionine | ARA_SEED_2 | p-USP | GC | 21 | 24 |
| | targeted | | | | | | | |
| 7081 | non- | B0161 | methionine | ARA_LEAF | p-Super | GC | 71 | 182 |
| | targeted | | | | | | | |
| 7269 | plastidic | B0348 | methionine | ARA_SEED_2 | p-USP | GC | 21 | 57 |
| 7333 | non- | B0449 | methionine | ARA_LEAF | p-Super | GC | 48 | 194 |
| | targeted | | | | | | | |
| 7686 | non- | B0486 | methionine | ARA_LEAF | p-Super | GC | 32 | 59 |
| | targeted | | | | | | | |
| 7730 | plastidic | B0870 | methionine | ARA_SEED_2 | p-USP | GC | 17 | 38 |
| 7917 | non- | B0898 | methionine | ARA_LEAF | p-Super | GC | 36 | 1310 |
| | targeted | | | | | | | |
| 7941 | non- | B1003 | methionine | ARA_LEAF | p-Super | GC | 49 | 144 |
| | targeted | | | | | | | |
| 7947 | non- | B1522 | methionine | ARA_LEAF | p-Super | GC | 42 | 144 |
| | targeted | | | | | | | |
| 7992 | non- | B1601 | methionine | ARA_LEAF | p-Super | GC | 41 | 64 |
| | targeted | | | | | | | |
| 8033 | plastidic | B1814 | methionine | ARA_SEED_2 | p-USP | GC | 19 | 59 |
| 8316 | non- | B1838 | methionine | ARA_LEAF | p-Super | LC | 50 | 59 |
| | targeted | | | | | | | |
| 8363 | plastidic | B1854 | methionine | ARA_SEED_2 | p-USP | GC | 19 | 45 |
| 8920 | non- | B2032 | methionine | ARA_LEAF | p-Super | GC | 37 | 75 |
| | targeted | | | | | | | |
| 8937 | mito- | B2066 | methionine | ARA_LEAF | p-Super | GC | 35 | 64 |
| | chondrial | | | | | | | |
| 9156 | non- | B2345 | methionine | ARA_LEAF | p-Super | LC | 41 | 231 |
| | targeted | | | | | | | |
| 9167 | non- | B2513 | methionine | ARA_LEAF | p-Super | GC | 39 | 55 |
| | targeted | | | | | | | |
| 9244 | non- | B2673 | methionine | ARA_LEAF | p-Super | LC | 32 | 57 |
| | targeted | | | | | | | |
| 9333 | non- | B2923 | methionine | ARA_LEAF | p-Super | GC | 32 | 54 |
| | targeted | | | | | | | |
| 9470 | non- | B3246 | methionine | ARA_LEAF | p-Super | LC | 36 | 46 |
| | targeted | | | | | | | |
| 9492 | non- | B3256 | methionine | ARA_SEED_2 | p-USP | GC | 77 | 121 |
| | targeted | | | | | | | |
| 10104 | non- | B3346 | methionine | ARA_LEAF | p-Super | LC | 37 | 40 |
| | targeted | | | | | | | |
| 10172 | plastidic | B3572 | methionine | ARA_SEED_2 | p-USP | GC | 20 | 652 |
| 10252 | non- | B3771 | methionine | ARA_SEED_2 | p-USP | GC | 22 | 93 |
| | targeted | | | | | | | |
| 10708 | non- | B3817 | methionine | ARA_LEAF | p-Super | LC | 37 | 61 |
| | targeted | | | | | | | |
| 10726 | non- | B3823 | methionine | ARA_LEAF | p-Super | GC | 38 | 48 |
| | targeted | | | | | | | |
| 10740 | non- | B4029 | methionine | ARA_LEAF | p-Super | GC | 84 | 691 |
| | targeted | | | | | | | |
| 10811 | non- | GM02 | methionine | ARA_LEAF | p-PcUBI | LC | 37 | 55 |
| | targeted | LC12622 | | | | | | |
| 11211 | mito- | Sll1108 | methionine | ARA_LEAF | p-PcUBI | LC | 39 | 67 |
| | chondrial | | | | | | | |
| 11423 | mito- | Sll1545 | methionine | ARA_LEAF | p-PcUBI | LC | 38 | 58 |
| | chondrial | | | | | | | |
| 11471 | mito- | Sll1917 | methionine | ARA_LEAF | p-PcUBI | LC | 30 | 34 |
| | chondrial | | | | | | | |
| 11990 | non- | Slr0338 | methionine | ARA_LEAF | p-PcUBI | LC | 29 | 40 |
| | targeted | | | | | | | |
| 11990 | plastidic | Slr0338 | methionine | ARA_LEAF | p-PcUBI | LC | 27 | 37 |
| 12070 | mito- | Slr1655 | methionine | ARA_LEAF | p-PcUBI | LC | 30 | 58 |
| | chondrial | | | | | | | |
| 12140 | plastidic | Slr1791 | methionine | ARA_LEAF | p-PcUBI | LC | 38 | 66 |
| 12341 | plastidic | Slr2072 | methionine | ARA_LEAF | p-PcUBI | GC | 36 | 74 |
| 12698 | non- | TTC0019 | methionine | ARA_LEAF | p-PcUBI | GC | 35 | 71 |
| | targeted | | | | | | | |
| 12974 | non- | TTC1550 | methionine | ARA_LEAF | p-PcUBI | GC | 36 | 44 |
| | targeted | | | | | | | |
| 13376 | non- | Yal012w | methionine | ARA_SEED_2 | p-PcUBI | GC | 22 | 31 |
| | targeted | | | | | | | |
| 14171 | non- | Ydl155w | methionine | ARA_LEAF | Big35S | GC | 34 | 107 |
| | targeted | | | | | | | |
| 14275 | plastidic | Ydl159w | methionine | ARA_LEAF | p-Super | GC | 44 | 54 |
| 14302 | plastidic | Ydl168w | methionine | ARA_LEAF | p-Super | GC | 40 | 46 |
| 14706 | plastidic | Ydr131c | methionine | ARA_LEAF | p-Super | GC | 30 | 31 |
| 14715 | plastidic | Yhl013c | methionine | ARA_LEAF | p-Super | GC | 32 | 73 |
| 14769 | plastidic | Yil053w | methionine | ARA_LEAF | p-Super | GC | 26 | 31 |
| 14821 | plastidic | Yjl137c | methionine | ARA_LEAF | p-Super | GC | 26 | 37 |
| 14843 | non- | Yjr130c | methionine | ARA_LEAF | Big35S | GC | 24 | 30 |
| | targeted | | | | | | | |
| 14885 | plastidic | Yjr139c | methionine | ARA_LEAF | p-Super | LC | 38 | 59 |
| 15175 | plastidic | Yml084w | methionine | ARA_LEAF | p-Super | LC | 31 | 52 |
| 15179 | plastidic | Yol160w | methionine | ARA_LEAF | p-Super | LC | 31 | 47 |
| 15183 | plastidic | Yor392w | methionine | ARA_LEAF | p-Super | LC | 14 | 20 |

Column 1 shows the SEQ ID NO, Column 2 shows the expression type (targeted or non-targeted), Column 3 shows the “gene name” (locus), Column 4 shows the metabolite analyzed, Column 5 indicates the A. thaliana source tissue analyzed, Column 6 indicates the used promoter for expression, Column 7 indicates the analytical method. Columns 8 and 9 show the minumum and the maximum increase of the analyzed metabolite (in percent) in comparison to the wild type (=ratio_by_weight, given as percent increase).

When the analyses were repeated independently, all results proved to be significant.

### Example 12

### Engineering Arabidopsis Plants with an Increased Production of a Fine Chemical by (Over) Expressing a FCRP-Protein Encoded by a Gene from Saccharomyces Cerevisiae or Synechocystis or E. Coli, Azotobacter Vinelandii, Thermus Thermophilus, Physcomitrella Patens, Arabidopsis. Thaliana, Brassica Napus, Glycine Max, Zea Mays or Oryza Sativa using Tissue-Specific and/or Stress Inducible Promoters

Transgenic Arabidopsis plants are created as in example 11 to express the FCRP under the control of a tissue-specific and/or stress inducible promoter.

T2 generation plants are produced and are grown under standard conditions. The fine chemical production is determined after a total time of 29 to 30 days starting with the sowing. The transgenic Arabidopsis plant produces more fine chemical then non-transgenic control plants.

### Example 13

### Engineering Alfalfa Plants with an Increased Production of a Fine Chemical by (Over)Expressing a FCRP-Protein Encoded by a gene from Saccharomyces Cerevisiae or Synechocystis or E. Coli, Azotobacter Vinelandii, Thermus Thermophilus, Physcomitrella Patens, Arabidopsis. Thaliana, Brassica Napus, Glycine Max, Zea Mays or Oryza Sativa

A regenerating clone of alfalfa (Medicago sativa) is transformed using the method of (McKersie et al., Plant Physiol 119, 839 (1999)). Regeneration and transformation of alfalfa is genotype dependent and therefore a regenerating plant is required. Methods to obtain regenerating plants have been described. For example, these can be selected from the cultivar Rangelander (Agriculture Canada) or any other commercial alfalfa variety as described by Brown D. C. W. and Atanassov A. (Plant Cell Tissue Organ Culture 4, 111 (1985)). Alternatively, the RA3 variety (University of Wisconsin) is selected for use in tissue culture (Walker et al., Am. J. Bot. 65, 654 (1978)).

Petiole explants are cocultivated with an overnight culture of Agrobacterium tumefaciens C58C1 pMP90 (McKersie et al., Plant Physiol 119, 839 (1999)) or LBA4404 containing a binary vector. Many different binary vector systems have been described for plant transformation (e.g. G. An, in “Agrobacterium Protocols, Methods in Molecular Biology”, Vol 44, pp. 47-62, Gartland K. M. A.and Davey M. R., eds. Humana Press, Totowa, N.J.). Many are based on the vector pBIN19 described by Bevan (Nucleic Acid Research. 12, 8711 (1984)) that includes a plant gene expression cassette flanked by the left and right border sequences from the Ti plasmid of Agrobacterium tumefaciens. A plant gene expression cassette consists of at least two genes—a selection marker gene and a plant promoter regulating the transcription of the cDNA or genomic DNA of the trait gene. Various selection marker genes can be used including the Arabidopsis gene encoding a mutated acetohydroxy acid synthase (AHAS) enzyme (U.S. Pat. No. 5,7673,666 and U.S. Pat. No. 6,225,105). Similarly, various promoters can be used to regulate the trait gene that provides constitutive, developmental, tissue or environmental regulation of gene transcription. In this example, the 34S promoter (GenBank Accession numbers M59930 and X16673) is used to provide constitutive expression of the trait gene.

The explants are cocultivated for 3 days in the dark on SH induction medium containing 288 mg/ L Pro, 53 mg/ L thioproline, 4.35 g/l K2SO4, and 100 μm acetosyringinone. The explants are washed in half-strength Murashige-Skoog medium (Murashige and Skoog, 1962) and plated on the same SH induction medium without acetosyringinone but with a suitable selection agent and suitable antibiotic to inhibit Agrobacterium growth. After several weeks, somatic embryos are transferred to BOi2Y development medium containing no growth regulators, no antibiotics, and 50 g/l sucrose. Somatic embryos are subsequently germinated on half-strength Murashige-Skoog medium. Rooted seedlings are transplanted into pots and grown in a greenhouse.

T1 or T2 generation plants are produced, grown and processed and evaluated for their fine chemical produced in analogy as described above.

### Example 14

### Engineering Ryegrass Plants with an Increased Production of Fine Chemical by (Over)-Expressing a FCRP Encoded by a Gene from Saccharomyces Cerevisiae or Synechocystis or E. Coli, Azotobacter Vinelandii, Thermus Thermophilus, Physcomitrella Patens, Arabidopsis. Thaliana, Brassica Napus, Glycine Max, Zea Mays or Oryza Sativa

Seeds of several different ryegrass varieties may be used as explant sources for transformation, including the commercial variety Gunne available from Svalof Weibull seed company or the variety Affinity. Seeds are surface-sterilized sequentially with 1% Tween-20 for 1 minute, 100% bleach for 60 minutes, 3 rinses with 5 minutes each with deionized and distilled H₂O, and then germinated for 3-4 days on moist, sterile filter paper in the dark. Seedlings are further sterilized for 1 minute with 1% Tween-20, 5 minutes with 75% bleach, and rinsed 3 times with double distilled H₂O, 5 min each.

Surface-sterilized seeds are placed on the callus induction medium containing Murashige and Skoog basal salts and vitamins, 20 g/l sucrose, 150 mg/l asparagine, 500 mg/l casein hydrolysate, 3 g/l Phytagel, 10 mg/l BAP, and 5 mg/l dicamba. Plates are incubated in the dark at 25° C. for 4 weeks for seed germination and embryogenic callus induction.

After 4 weeks on the callus induction medium, the shoots and roots of the seedlings are trimmed away, the callus is transferred to fresh media, maintained in culture for another 4 weeks, and then transferred to MSO medium in light for 2 weeks. Several pieces of callus (11-17 weeks old) are either strained through a 10 mesh sieve and put onto callus induction medium, or cultured in 100 ml of liquid ryegrass callus induction media (same medium as for callus induction with agar) in a 250 ml flask. The flask is wrapped in foil and shaken at 175 rpm in the dark at 23° C. for 1 week. Sieving the liquid culture with a 40-mesh sieve collected the cells. The fraction collected on the sieve is plated and cultured on solid ryegrass callus induction medium for 1 week in the dark at 25° C. The callus is then transferred to and cultured on MS medium containing 1% sucrose for 2 weeks.

Transformation can be accomplished with either Agrobacterium of with particle bombardment methods. An expression vector is created containing a constitutive plant promoter and the cDNA of the gene in a pUC vector. The plasmid DNA is prepared from E. coli cells using with Qiagen kit according to manufacturer's instruction. Approximately 2 g of embryogenic callus is spread in the center of a sterile filter paper in a Petri dish. An aliquot of liquid MSO with 10 g/L sucrose is added to the filter paper. Gold particles (1.0 pm in size) are coated with plasmid DNA according to method of Sanford et al., 1993 and delivered to the embryogenic callus with the following parameters: 500 pg particles and 2 μg DNA per shot, 1300 psi and a target distance of 8.5 cm from stopping plate to plate of callus and 1 shot per plate of callus.

After the bombardment, calli are transferred back to the fresh callus development medium and maintained in the dark at room temperature for a 1-week period. The callus is then transferred to growth conditions in the light at 25° C. to initiate embryo differentiation with the appropriate selection agent, e.g. 250 nM Arsenal, 5 mg/l PPT or 50 mg/l kanamycin. Shoots resistant to the selection agent are appearing and once rooted are transferred to soil.

Samples of the primary transgenic plants (TO) are analyzed by PCR to confirm the presence of T-DNA. These results are confirmed by Southern hybridization in which DNA is electrophoresed on a 1% agarose gel and transferred to a positively charged nylon membrane (Roche Diagnostics). The PCR DIG Probe Synthesis Kit (Roche Diagnostics) is used to prepare a digoxigenin-labelled probe by PCR, and used as recommended by the manufacturer.

Transgenic T0 ryegrass plants are propagated vegetatively by excising tillers. The transplanted tillers are maintained in the greenhouse for 2 months until well established. The shoots are defoliated and allowed to grow for 2 weeks.

T1 or T2 generation plants are produced, grown and processed and evaluated for their fine chemical produced in analogy as described above to determine the fine chemical content.

### Example 15

### Engineering Soybean Plants with an Increased Production of Fine Chemical by (Over)-Expressing a FCRP Encoded by a Gene from Saccharomyces Cerevisiae or Synechocystis or E. Coli, Azotobacter Vinelandii, Thermus Thermophilus, Physcomitrella Patens, Arabidopsis. Thaliana, Brassica Napus, Glycine Max, Zea Mays or Oryza Sativa

Soybean is transformed according to the following modification of the method described in the Texas A&M patent U.S. Pat. No. 5,164,310. Several commercial soybean varieties are amenable to transformation by this method. The cultivar Jack (available from the Illinois Seed Foundation) is commonly used for transformation. Seeds are sterilized by immersion in 70% (v/v) ethanol for 6 min and in 25% commercial bleach (NaOCI) supplemented with 0.1% (v/v) Tween for 20 min, followed by rinsing 4 times with sterile double distilled water. Seven-day seedlings are propagated by removing the radicle, hypocotyl and one cotyledon from each seedling. Then, the epicotyl with one cotyledon is transferred to fresh germination media in petri dishes and incubated at 25 ° C. under a 16-h photoperiod (approx. 100 μmol m⁻2 s⁻1) for three weeks. Axillary nodes (approx. 4 mm in length) were cut from 3-4 week-old plants. Axillary nodes are excised and incubated in Agrobacterium LBA4404 culture.

Many different binary vector systems have been described for plant transformation (e.g. An G., in Agrobacterium Protocols. Methods in Molecular Biology Vol. 44, pp. 47-62, Gartland K. M. A.

and Davey M. R. eds. Humana Press, Totowa, N.J.). Many are based on the vector pBIN19 described by Bevan (Nucleic Acid Research. 12, 8711 (1984)) that includes a plant gene expression cassette flanked by the left and right border sequences from the Ti plasmid of Agrobacterium tumefaciens. A plant gene expression cassette consists of at least two genes—a selection marker gene and a plant promoter regulating the transcription of the cDNA or genomic DNA of the trait gene. Various selection marker genes can be used including the Arabidopsis gene encoding a mutated acetohydroxy acid synthase (AHAS) enzyme (U.S. Pat. No. 5,7673,666 and U.S. Pat. No. 6,225,105). Similarly, various promoters can be used to regulate the trait gene to provide constitutive, developmental, tissue or environmental regulation of gene transcription. In this example, the 34S promoter (GenBank Accession numbers M59930 and X16673) can be used to provide constitutive expression of the trait gene.

After the co-cultivation treatment, the explants are washed and transferred to selection media supplemented with 500 mg/l timentin. Shoots are excised and placed on a shoot elongation medium. Shoots longer than 1 cm are placed on rooting medium for two to four weeks prior to transplanting to soil.

The primary transgenic plants (T0) are analyzed by PCR to confirm the presence of T-DNA. These results are confirmed by Southern hybridization in which DNA is electrophoresed on a 1% agarose gel and transferred to a positively charged nylon membrane (Roche Diagnostics). The PCR DIG Probe Synthesis Kit (Roche Diagnostics) is used to prepare a digoxigenin-labelled probe by PCR, and used as recommended by the manufacturer.

T1 or T2 generation plants are produced, grown and processed and evaluated for their fine chemical produced in analogy as described above.

### Example 16

### Engineering Rapeseed/Canola Plants with an Increased Production of Fine Chemical by (Over)-Expressing a FCRP Encoded by a Gene from Saccharomyces Cerevisiae or Synechocystis or E. Coli, Azotobacter Vinelandii, Thermus Thermophilus, Physcomitrella Patens, Arabidopsis. Thaliana, Brassica Napus, Glycine Max, Zea Mays or Oryza Sativa

Cotyledonary petioles and hypocotyls of 5-6 day-old young seedlings are used as explants for tissue culture and transformed according to Babic et al. (Plant Cell Rep 17, 183 (1998)). The commercial cultivar Westar (Agriculture Canada) is the standard variety used for transformation, but other varieties can be used.

Agrobacterium tumefaciens LBA4404 containing a binary vector can be used for canola transformation. Many different binary vector systems have been described for plant transformation (e.g. An G., in Agrobacterium Protocols. Methods in Molecular Biology Vol. 44, p. 47-62, Gartland K. M. A. and Davey M. R. eds. Humana Press, Totowa, N.J.). Many are based on the vector pBIN19 described by Bevan (Nucleic Acid Research. 12, 8711(1984)) that includes a plant gene expression cassette flanked by the left and right border sequences from the Ti plasmid of Agrobacterium tumefaciens. A plant gene expression cassette consists of at least two genes—a selection marker gene and a plant promoter regulating the transcription of the cDNA or genomic DNA of the trait gene. Various selection marker genes can be used including the Arabidopsis gene encoding a mutated acetohydroxy acid synthase (AHAS) enzyme (U.S. Pat. No. 5,7673,666 and U.S. Pat. No. 6,225,105). Similarly, various promoters can be used to regulate the trait gene to provide constitutive, developmental, tissue or environmental regulation of gene transcription. In this example, the 34S promoter (GenBank Accession numbers M59930 and X16673) can be used to provide constitutive expression of the trait gene.

Canola seeds are surface-sterilized in 70% ethanol for 2 min., and then in 30% Clorox with a drop of Tween-20 for 10 min, followed by three rinses with sterilized distilled water. Seeds are then germinated in vitro 5 days on half strength MS medium without hormones, 1% sucrose, 0.7% Phytagar at 23° C., 16 h light. The cotyledon petiole explants with the cotyledon attached are excised from the in vitro seedlings, and inoculated with Agrobacterium by dipping the cut end of the petiole explant into the bacterial suspension. The explants are then cultured for 2 days on MSBAP-3 medium containing 3 mg/l BAP, 3% sucrose, 0.7% Phytagar at 23° C., 16 h light. After two days of co-cultivation with Agrobacterium, the petiole explants are transferred to MSBAP-3 medium containing 3 mg/l BAP, cefotaxime, carbenicillin, or timentin (300 mg/l) for 7 days, and then cultured on MSBAP-3 medium with cefotaxime, carbenicillin, or timentin and selection agent until shoot regeneration. When the shoots were 5-10 mm in length, they are cut and transferred to shoot elongation medium (MSBAP-0.5, containing 0.5 mg/l BAP). Shoots of about 2 cm in length are transferred to the rooting medium (MSO) for root induction.

Samples of the primary transgenic plants (T0) are analyzed by PCR to confirm the presence of T-DNA. These results are confirmed by Southern hybridization in which DNA is electrophoresed on a 1% agarose gel and transferred to a positively charged nylon membrane (Roche Diagnostics). The PCR DIG Probe Synthesis Kit (Roche Diagnostics) is used to prepare a digoxigenin-labelled probe by PCR, and used as recommended by the manufacturer.

T1 or T2 generation plants are produced, grown and processed and evaluated for their fine chemical produced in analogy as described above.

### Example 17

### Engineering Corn Plants with an Increased Production of Fine Chemical by (Over)-Expressing a FCRP Encoded by a Gene from Saccharomyces Cerevisiae or Synechocystis or E. Coli, Azotobacter Vinelandii, Thermus Thermophilus, Physcomitrella Patens, Arabidopsis. Thaliana, Brassica Napus, Glycine Max, Zea Mays or Oryza Sativa

Transformation of maize (Zea Mays L.) is performed with a modification of the method described by Ishida et al. (Nature Biotech 14745 (1996)). Transformation is genotype-dependent in corn and only specific genotypes are amenable to transformation and regeneration. The inbred line A188 (University of Minnesota) or hybrids with A188 as a parent are good sources of donor material for transformation (Fromm et al., Biotech 8, 833 (1990)), but other genotypes can be used successfully as well. Ears are harvested from corn plants at approximately 11 days after pollination (DAP) when the length of immature embryos is about 1 to 1.2 mm. Immature embryos are co-cultivated with Agrobacterium tumefaciens that carry “super binary” vectors and transgenic plants are recovered through organogenesis. The super binary vector system of Japan Tobacco is described in WO patents WO 94/00977 and WO 95/06722. Vectors were constructed as described. Various selection marker genes can be used including the maize gene encoding a mutated acetohydroxy acid synthase (AHAS) enzyme (U.S. Pat. No. 6,025,541). Similarly, various promoters can be used to regulate the trait gene to provide constitutive, developmental, tissue or environmental regulation of gene transcription. In this example, the 34S promoter (GenBank Accession numbers M59930 and X16673) was used to provide constitutive expression of the trait gene.

Excised embryos are grown on callus induction medium, then maize regeneration medium, containing imidazolinone as a selection agent. The Petri plates are incubated in the light at 25 ° C. for 2-3 weeks, or until shoots develop. The green shoots are transferred from each embryo to maize rooting medium and incubated at 25° C. for 2-3 weeks, until roots develop. The rooted shoots are transplanted to soil in the greenhouse. T1 seeds are produced from plants that exhibit tolerance to the imidazolinone herbicides and which are PCR positive for the transgenes.

The T1 transgenic plants are then evaluated for their fine chemical producted as described above. The T1 generation of single locus insertions of the T-DNA will segregate for the transgene in a 3:1 ratio. Those progeny containing one or two copies of the transgene are tolerant regarding the imidazolinone herbicide, and exhibit an increased fine chemical production then those progeny lacking the transgenes.

T1 or T2 generation plants are produced, grown, processed and evaluated for their fine chemical produced in analogy as described above. Homozygous T2 plants exhibited similar phenotypes. Hybrid plants (F1 progeny) of homozygous transgenic plants and non-transgenic plants also exhibited enhanced fine chemical production.

### Example 18

### Engineering Wheat Plants with an Increased Production of Fine Chemical by (Over)-Expressing a FCRP Encoded by a Gene from Saccharomyces Cerevisiae or Synechocystis or E. Coli, Azotobacter Vinelandii, Thermus Thermophilus, Physcomitrella Patens, Arabidopsis. Thaliana, Brassica Napus, Glycine Max, Zea Mays or Oryza Sativa

Transformation of wheat is performed with the method described by Ishida et al. (Nature Biotech. 14745 (1996)). The cultivar Bobwhite (available from CYMMIT, Mexico) is commonly used in transformation. Immature embryos are co-cultivated with Agrobacterium tumefaciens that carry “super binary” vectors, and transgenic plants are recovered through organogenesis. The super binary vector system of Japan Tobacco is described in WO patents WO 94/00977 and WO 95/06722. Vectors were constructed as described. Various selection marker genes can be used including the maize gene encoding a mutated acetohydroxy acid synthase (AHAS) enzyme (U.S. Pat. No. 6,025,541). Similarly, various promoters can be used to regulate the trait gene to provide constitutive, developmental, tissue or environmental regulation of gene transcription. In this example, the 34S promoter (GenBank Accession numbers M59930 and X16673) was used to provide constitutive expression of the trait gene.

After incubation with Agrobacterium, the embryos are grown on callus induction medium, then regeneration medium, containing imidazolinone as a selection agent. The Petri plates are incubated in the light at 25 ° C. for 2-3 weeks, or until shoots develop. The green shoots are transferred from each embryo to rooting medium and incubated at 25 ° C. for 2-3 weeks, until roots develop. The rooted shoots are transplanted to soil in the greenhouse. T1 seeds are produced from plants that exhibit tolerance to the imidazolinone herbicides and which are PCR positive for the transgenes.

The T1 transgenic plants are then evaluated for their enhanced production of fine chemical according to the methods described in the previous examples. The T1 generation of single locus insertions of the T-DNA will segregate for the transgene in a 3:1 ratio. Those progeny containing one or two copies of the transgene are tolerant regarding the imidazolinone herbicide, and exhibit an enhanced production of fine chemical then those progeny lacking the transgenes. Homozygous T2 plants exhibited similar phenotypes.

### Example 19

### Engineering Rice Plants with an Increased Production of Fine Chemical by (Over)-Expressing a FCRP Encoded by a Gene from Saccharomyces Cerevisiae or Synechocystis or E. Coli, Azotobacter Vinelandii, Thermus Thermophilus, Physcomitrella Patens, Arabidopsis. Thaliana, Brassica Napus, Glycine Max, Zea Mays or Oryza Sativa

The Agrobacterium containing the expression vector of the invention is used to transform Oryza sativa plants. Mature dry seeds of the rice japonica cultivar Nipponbare are dehusked. Sterilization is carried out by incubating for one minute in 70% ethanol, followed by 30 minutes in 0.2% HgCl₂, followed by a 6 times 15 minutes can beh with sterile distilled water. The sterile seeds are then germinated on a medium containing 2,4-D (callus induction medium). After incubation in the dark for four weeks, embryogenic, scutellum-derived calli are excised and propagated on the same medium. After two weeks, the calli are multiplied or propagated by subculture on the same medium for another 2 weeks. Embryogenic callus pieces are sub-cultured on fresh medium 3 days before co-cultivation (to boost cell division activity).

Agrobacterium strain LBA4404 containing the expression vector of the invention is used for cocultivation. Agrobacterium is inoculated on AB medium with the appropriate antibiotics and cultured for 3 days at 28° C. The bacteria are then collected and suspended in liquid co-cultivation medium to a density (OD600) of about 1. The suspension is then transferred to a Petri dish and the calli immersed in the suspension for 15 minutes. The callus tissues are then blotted dry on a filter paper and transferred to solidified, co-cultivation medium and incubated for 3 days in the dark at 25° C. Co-cultivated calli are grown on 2,4-D-containing medium for 4 weeks in the dark at 28° C. in the presence of a selection agent. During this period, rapidly growing resistant callus islands developed. After transfer of this material to a regeneration medium and incubation in the light, the embryogenic potential is released and shoots developed in the next four to five weeks. Shoots are excised from the calli and incubated for 2 to 3 weeks on an auxin-containing medium from which they are transferred to soil. Hardened shoots are grown under high humidity and short days in a greenhouse.

Approximately 35 independent TO rice transformants are generated for one construct. The primary transformants are transferred from a tissue culture chamber to a greenhouse. After a quantitative PCR analysis to verify copy number of the T-DNA insert, only single copy transgenic plants that exhibit tolerance to the selection agent are kept for harvest of T1 seed. Seeds are then harvested three to five months after transplanting. The method yielded single locus transformants at a rate of over 50% (Aldemita and Hodges1996, Chan et al. 1993, Hiei et al. 1994).

T1 or T2 generation plants are produced, grown, processed and evaluated for their fine chemical produced in analogy as described above

### Example 20

### Identification of Identical and Heterologous Genes

Gene sequences can be used to identify identical or heterologous genes from cDNA or genomic libraries. Identical genes (e. g. full-length cDNA clones) can be isolated via nucleic acid hybridization using for example cDNA libraries. Depending on the abundance of the gene of interest, 100,000 up to 1,000,000 recombinant bacteriophages are plated and transferred to nylon membranes. After denaturation with alkali, DNA is immobilized on the membrane by e. g. UV cross linking. Hybridization is carried out at high stringency conditions. In aqueous solution, hybridization and washing is performed at an ionic strength of 1 M NaCl and a temperature of 68° C. Hybridization probes are generated by e.g. radioactive (32P) nick transcription labeling (High Prime, Roche, Mannheim, Germany). Signals are detected by autoradiography.

Partially identical or heterologous genes that are related but not identical can be identified in a manner analogous to the above-described procedure using low stringency hybridization and washing conditions. For aqueous hybridization, the ionic strength is normally kept at 1 M NaCl while the temperature is progressively lowered from 68 to 42° C.

Isolation of gene sequences with homology (or sequence identity/similarity) only in a distinct domain of (for example 10-20 amino acids) can be carried out by using synthetic radio labeled oligonucleotide probes. Radiolabeled oligonucleotides are prepared by phosphorylation of the 5-prime end of two complementary oligonucleotides with T4 polynucleotide kinase. The complementary oligonucleotides are annealed and ligated to form concatemers. The double stranded concatemers are than radiolabeled by, for example, nick transcription. Hybridization is normally performed at low stringency conditions using high oligonucleotide concentrations.

### Oligonucleotide Hybridization Solution:

6×SSC
0.01 M sodium phosphate
1 mM EDTA (pH 8)
0.5% SDS
100 pg/ml denatured salmon sperm DNA
0.1% nonfat dried milk

During hybridization, temperature is lowered stepwise to 5-10° C. below the estimated oligonucleotide Tm or down to room temperature followed by washing steps and autoradiography. Washing is performed with low stringency such as 3 washing steps using 4×SSC. Further details are described by Sambrook J. et al., 1989, “Molecular Cloning: A Laboratory Manual,” Cold Spring Harbor Laboratory Press or Ausubel F. M. et al., 1994, “Current Protocols in Molecular Biology,” John Wiley & Sons.

### Example 21

### Identification of Identical Genes by Screening Expression Libraries with Antibodies

c-DNA clones can be used to produce recombinant polypeptide for example in E. coli (e.g. Qiagen QIAexpress pQE system). Recombinant polypeptides are then normally affinity purified via Ni-NTA affinity chromatography (Qiagen). Recombinant polypeptides are then used to produce specific antibodies for example by using standard techniques for rabbit immunization. Antibodies are affinity purified using a Ni-NTA column saturated with the recombinant antigen as described by Gu et al., BioTechniques 17, 257 (1994). The antibody can than be used to screen expression cDNA libraries to identify identical or heterologous genes via an immunological screening (Sambrook J., et al., “Molecular Cloning: A Laboratory Manual,” Cold Spring Harbor Laboratory Press, 1989, or Ausubel F. M. et al., “Current Protocols in Molecular Biology”, John Wiley & Sons, 1994,).

### FIGURES

FIG. 1. Vector VC-MME220-1 qcz (SEQ ID NO: 41) used for cloning gene of interest for non-targeted expression.

FIG. 2. Vector VC-MME221-1qcz (SEQ ID NO: 46) used for cloning gene of interest for non-targeted expression.

FIG. 3. Vector VC-MME354-1 QCZ (SEQ ID NO: 32) used for cloning gene of interest for plastidic targeted expression.

FIG. 4. Vector VC-MME432-1 qcz (SEQ ID NO: 42) used for cloning gene of interest for plastidic targeted expression.

FIG. 5. Vector VC-MME489-1 QCZ (SEQ ID NO: 56) used for cloning gene of interest for non-targeted expression and cloning of a targeting sequence.

FIG. 6. Vector pMTX0270p (SEQ ID NO: 30) used for cloning of a targeting sequence.

FIG. 7. Vector pMTX155 (SEQ ID NO: 31) used for used for cloning gene of interest for non-targeted expression.

FIG. 8. Vector VC-MME356-1 QCZ (SEQ ID NO: 34) used for mitochondric targeted expression.

FIG. 9. Vector VC-MME301-1 QCZ (SEQ ID NO: 36) used for non-targeted expression in preferentially seeds.

FIG. 10. Vector pMTX461korrp (SEQ ID NO: 37) used for plastidic targeted expression in preferentially seeds.

FIG. 11. Vector VC-MME462-1QCZ (SEQ ID NO: 39) used for mitochondric targeted expression in preferentially seeds.

FIG. 12. Vector VC-MME431-1qcz (SEQ ID NO: 44) used for mitochondric targeted expression.

FIG. 13. Vector pMTX447korr (SEQ ID NO: 47) used for plastidic targeted expression.

FIG. 14. Vector VC-MME445-1qcz (SEQ ID NO: 49) used for mitochondric targeted expression.

FIG. 15. Vector VC-MME289-1qcz (SEQ ID NO: 51) used for non targeted expression in preferentially seeds.

FIG. 16. Vector VC-MME464-1qcz (SEQ ID NO: 52) used for plastidic targeted expression in preferentially seeds.

FIG. 17. Vector VC-MME465-1 qcz (SEQ ID NO: 54) used for mitochondric targeted expression in preferentially seeds.

In a further embodiment, the present invention relates in paragraphs [0000.1.1.2] to [0514.1.1.2] to a further process for the production of at least a, preferably a, fine chemical selected from the group consisting of arginine, glutamate, glutamine and proline, as defined below and corresponding embodiments as described herein as follows

[0001.1.1.2] to [0011.1.1.2] for the disclosure of these paragraphs see [0001.1.1.1] to [0011.1.1.1] above.

It is an object of the present invention to develop an inexpensive process for the synthesis of arginine, glutamate, glutamine and/or proline.

for the disclosure of this paragraph see [0013.1.1.1] above.

Accordingly, in a first embodiment, the invention relates to a process for the production of at least one, preferably a, fine chemical selected from the group consisting of:

arginine, glutamate, glutamine and proline, or, in other words, of the “fine chemical” or “fine chemical of the invention”.

The terms “fine chemical of the invention”, “fine chemical” or “the fine chemical” are used herein equally and relate in context of the paragraphs or sections [0014.1.2.2] to [0514.1.1.2] essentially to the metabolite or the metabolites indicated in column 7, application no. 2 of Tables I, II and IV in the respective line.

Further, the term “in context of any of the paragraphs [0014.1.2.2] to [0514.1.1.2]” as used herein means that for any of said paragraphs [0014.1.2.2] to [0514.1.1.2] the term “the fine chemical” is understood to follow the definition of paragraphs or sections [0014.1.2.2] and [0015.1.2.2], independently whether it refers to any other paragraph or not and whether the reference recites the term “fine chemical” in an other context.

Thus, in cases where one or more paragraphs or sections are incorporated by reference into any of the present paragraphs [0014.1.2.2] to [0514.1.1.2], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to the definition of paragraph [0014.1.2.2] and [0015.1.2.2].

Accordingly, in one embodiment, the term “fine chemical of the invention”, “fine chemical” or “the fine chemical” as used herein (alone or in combination, like in FCRP) means “arginine in context of the nucleic acid or polypeptide sequences listed in the respective same line of any one of Tables I to IV of application no. 2 and indicating in column 7 the metabolite “arginine”. In one embodiment, the term arginine or the term “fine chemical” mean in context of the paragraphs or sections [0014.1.2.2] to [0514.1.1.2] at least one chemical compound with an activity of the above mentioned arginine, respectively.

Accordingly, in one embodiment, the term “fine chemical of the invention”, “fine chemical” or “the fine chemical” as used herein (alone or in combination, like in FCRP) means “glutamate in context of the nucleic acid or polypeptide sequences listed in the respective same line of any one of Tables I to IV of application no. 2 and indicating in column 7 the metabolite “glutamate”. In one embodiment, the term glutamate or the term “fine chemical” mean in context of the paragraphs or sections [0014.1.2.2] to [0514.1.1.2] at least one chemical compound with an activity of the above mentioned glutamate, respectively.

Accordingly, in one embodiment, the term “fine chemical of the invention”, “fine chemical” or “the fine chemical” as used herein (alone or in combination, like in FCRP) means “glutamine in context of the nucleic acid or polypeptide sequences listed in the respective same line of any one of Tables I to IV of application no. 2 and indicating in column 7 the metabolite “glutamine”. In one embodiment, the term glutamine or the term “fine chemical” mean in context of the paragraphs or sections [0014.1.2.2] to [0514.1.1.2] at least one chemical compound with an activity of the above mentioned glutamine, respectively.

Accordingly, in one embodiment, the term “fine chemical of the invention”, “fine chemical” or “the fine chemical” as used herein (alone or in combination, like in FCRP) means “proline in context of the nucleic acid or polypeptide sequences listed in the respective same line of any one of Tables I to IV of application no. 2 and indicating in column 7 the metabolite “proline”. In one embodiment, the term proline or the term “fine chemical” mean in context of the paragraphs or sections [0014.1.2.2] to [0514.1.1.2] at least one chemical compound with an activity of the above mentioned proline, respectively.

Accordingly, in one embodiment, the terms “fine chemical of the invention”, “fine chemical” or “the fine chemical” mean in context of any of the paragraphs [0014.1.2.2] to [0514.1.1.2] arginine, preferably the L-enantiomer of arginine, its salts, ester or amides in free form or bound to proteins. In a preferred embodiment, the term “the fine chemical” means arginine or its salts, in free form or bound to proteins. In a preferred embodiment, the term “the fine chemical” means the L-enantiomer of arginine. On the other hand in case “arginine” is stated it means arginine itself, its salts, ester or amides in free form or bound to proteins, preferably the L-enantiomer of arginine, its salts, ester or amides in free form or bound to proteins. In a preferred embodiment “arginine” means the L-enantiomer of arginine in free form. In another preferred embodiment “arginine” means the L-enantiomer of arginine bound to proteins.

Accordingly, in one embodiment, the terms “fine chemical of the invention”, “fine chemical” or “the fine chemical” mean in context of any of the paragraphs [0014.1.2.2] to [0514.1.1.2] glutamate, preferably the L-enantiomer of glutamate, its salts, ester or amides in free form or bound to proteins. In a preferred embodiment, the term “the fine chemical” means glutamate or its salts, in free form or bound to proteins. In a preferred embodiment, the term “the fine chemical” means the L-enantiomer of glutamate.

On the other hand in case “glutamate” is stated it means glutamate itself, its salts, ester or amides in free form or bound to proteins, preferably the L-enantiomer of glutamate, its salts, ester or amides in free form or bound to proteins. In a preferred embodiment “glutamate” means the L-enantiomer of glutamate in free form. In another preferred embodiment “glutamate” means the L-enantiomer of glutamate bound to proteins.

Accordingly, in one embodiment, the terms “fine chemical of the invention”, “fine chemical” or “the fine chemical” mean in context of any of the paragraphs [0014.1.2.2] to [0514.1.1.2] glutamine, preferably the L-enantiomer of glutamine, its salts, ester or amides in free form or bound to proteins. In a preferred embodiment, the term “the fine chemical” means glutamine or its salts, in free form or bound to proteins. In a preferred embodiment, the term “the fine chemical” means the L-enantiomer of glutamine.

On the other hand in case “glutamine” is stated it means glutamine itself, its salts, ester or amides in free form or bound to proteins, preferably the L-enantiomer of glutamine, its salts, ester or amides in free form or bound to proteins. In a preferred embodiment “glutamine” means the L-enantiomer of glutamine in free form. In another preferred embodiment “glutamine” means the L-enantiomer of glutamine bound to proteins.

Accordingly, in one embodiment, the terms “fine chemical of the invention”, “fine chemical” or “the fine chemical” mean in context of any of the paragraphs [0014.1.2.2] to [0514.1.1.2] proline, preferably the L-enantiomer of proline, its salts, ester or amides in free form or bound to proteins. In a preferred embodiment, the term “the fine chemical” means proline or its salts, in free form or bound to proteins. In a preferred embodiment, the term “the fine chemical” means the Lenantiomer of proline.

On the other hand in case “proline” is stated it means proline itself, its salts, ester or amides in free form or bound to proteins, preferably the L-enantiomer of proline, its salts, ester or amides in free form or bound to proteins. In a preferred embodiment “proline” means the L-enantiomer of proline in free form. In another preferred embodiment “proline” means the L-enantiomer of proline bound to proteins.

Further, the term “in context of any of the paragraphs [0014.1.2.2] to [0514.1.1.2]” as used herein means that for any of said paragraphs [0014.1.2.2] to [0514.1.1.2] the term “the fine chemical” is understood to follow the definition of section [0014.1.2.2] or section [0015.1.2.2], independently whether it refers to any other paragraph or not and whether the reference recites the term “fine chemical” in an other context.

Thus, in cases where one or more paragraphs or sections are incorporated by reference into any of the present paragraphs [0014.1.2.2] to [0514.1.1.2], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to this definition of this paragraph [0015.1.2.2].

Further, the term “fine chemicals” as used herein relates to compositions comprising said fine chemical(s), i.e. comprising arginine, glutamate, glutamine, and/or proline, respectively.

Accordingly, the present invention relates to a process for the production of glutamate, which comprises
(a) increasing or generating one or more activities selected from the group consisting of 47266012-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of 47266012-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating one or more activities selected from the group consisting of 49747384_SOYBEAN-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamate, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of arginine, which comprises
(a) increasing or generating one or more activities selected from the group consisting of glucose-6-phosphate 1-dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating one or more activities selected from the group consisting of glucose-6-phosphate 1-dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of amino acid acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating one or more activities selected from the group consisting of At1g17440-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating one or more activities selected from the group consisting of At1g19800-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating one or more activities selected from the group consisting of cullin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating one or more activities selected from the group consisting of cullin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of At1 g29350-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of arginine, which comprises
(a) increasing or generating one or more activities selected from the group consisting of eukaryotic translation initiation factor 5 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating one or more activities selected from the group consisting of eukaryotic translation initiation factor 5 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of At1g47380-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of At1g67340-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.
Accordingly, the present invention relates to a process for the production of glutamine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of serine protease inhibitor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of CBL-interacting protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of CBL-interacting protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of CBL-interacting protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of delta-1-pyrroline 5-carboxylase synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of phosphatidylinositol 3- and 4-kinase family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of ankyrin repeat family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of arginine, which comprises
(a) increasing or generating one or more activities selected from the group consisting of thioredoxin family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating one or more activities selected from the group consisting of thioredoxin family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of thioredoxin family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of thioredoxin family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of harpin-induced family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating one or more activities selected from the group consisting of harpin-induced family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of harpin-induced family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of DNA binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of DNA mismatch repair protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of CCAAT-binding transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of glucose-6-phosphate 1-dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of monthiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of monthiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamate, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of At4g32480-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating one or more activities selected from the group consisting of At4g32480-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of At4g32480-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of arginine, which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating one or more activities selected from the group consisting of gibberellin 20-oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of integral membrane transporter family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of integral membrane transporter family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating one or more activities selected from the group consisting of At5g16650-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating one or more activities selected from the group consisting of At5g16650-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of At5g16650-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating one or more activities selected from the group consisting of translation initiation factor subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating one or more activities selected from the group consisting of zinc finger protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating one or more activities selected from the group consisting of zinc finger protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamate, which comprises
(a) increasing or generating one or more activities selected from the group consisting of adenylylsulfate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of adenylylsulfate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating one or more activities selected from the group consisting of malic enzyme in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism. Accordingly, the present invention relates to a process for the production of proline, which comprises
(a) increasing or generating one or more activities selected from the group consisting of malic enzyme in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of glycosyl transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of sec-independent protein translocase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of arginine, which comprises
(a) increasing or generating one or more activities selected from the group consisting of beta-hydroxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of arginine, which comprises
(a) increasing or generating one or more activities selected from the group consisting of elongation factor Tu in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating one or more activities selected from the group consisting of elongation factor Tu in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of elongation factor Tu in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating one or more activities selected from the group consisting of ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of acyl-CoA synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating one or more activities selected from the group consisting of acyl-CoA synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of acyl-CoA synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of acyl-CoA synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of 2-oxoglutarate dehydrogenase E1 subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of arginine, which comprises
(a) increasing or generating one or more activities selected from the group consisting of enoyl-CoA hydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating one or more activities selected from the group consisting of enoyl-CoA hydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of acyl-transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating one or more activities selected from the group consisting of lysyltRNA synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating one or more activities selected from the group consisting of 30S ribosomal protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamate, which comprises
(a) increasing or generating one or more activities selected from the group consisting of CTP synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of CTP synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating one or more activities selected from the group consisting of glucose-1-phosphate cytidylyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of glucose-1-phosphate cytidylyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of glucose-1-phosphate cytidylyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of aminotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating one or more activities selected from the group consisting of hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of glutamate-ammonia-ligase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating one or more activities selected from the group consisting of glutamate-ammonia-ligase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of glutamate-ammonia-ligase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of fumarylacetoacetate hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of HesB/YadR/YfhF family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating one or more activities selected from the group consisting of

HesB/YadR/YfhF family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of HesB/YadR/YfhF family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of L-aspartate oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of Chaperone protein CIpB in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of purine nucleoside phosphorylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating one or more activities selected from the group consisting of purine nucleoside phosphorylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of purine nucleoside phosphorylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of transcriptional regulator protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of AX653549-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of AX653549-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating one or more activities selected from the group consisting of AY087308-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating one or more activities selected from the group consisting of threonine synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating one or more activities selected from the group consisting of threonine synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of threonine synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of L-ribulose-5-phosphate 4-epimerase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of dihydrolipoamide acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating one or more activities selected from the group consisting of glucose dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating one or more activities selected from the group consisting of glucose dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of glucose dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of serine protease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of acyl-CoA dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating one or more activities selected from the group consisting of acyl-CoA dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating one or more activities selected from the group consisting of beta-galactosidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of ATP-binding component of a transport system in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of b0456-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of membrane transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating one or more activities selected from the group consisting of membrane transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of b0518-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating one or more activities selected from the group consisting of isochorismate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating one or more activities selected from the group consisting of isochorismate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of isochorismate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of isochorismate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of zinc transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating one or more activities selected from the group consisting of asparaginase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating one or more activities selected from the group consisting of Leucyl/phenylalanyl-tRNA-protein transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of Leucyl/phenylalanyl-tRNA-protein transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of major facilitator superfamily transporter protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating one or more activities selected from the group consisting of major facilitator superfamily transporter protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of DNA helicase IV in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of methylglyoxal synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of phosphoanhydride phosphorylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of b1003-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of b1003-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of lipoprotein precursor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating one or more activities selected from the group consisting of b1024-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of b1108-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of b1108-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of isocitrate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of b1137-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating one or more activities selected from the group consisting of b1137-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating one or more activities selected from the group consisting of b1137-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of b1163-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of sodium/proton antiporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of membrane protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of membrane protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of b1259-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating one or more activities selected from the group consisting of b1259-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating one or more activities selected from the group consisting of anthranilate synthase component II in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating one or more activities selected from the group consisting of b1280-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating one or more activities selected from the group consisting of glutamine synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating one or more activities selected from the group consisting of aldehyde dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamate, which comprises
(a) increasing or generating one or more activities selected from the group consisting of b1330-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of b1330-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating one or more activities selected from the group consisting of lipoprotein precursor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of lipoprotein precursor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating one or more activities selected from the group consisting of b1445-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of b1445-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating one or more activities selected from the group consisting of b1522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of acid shock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating one or more activities selected from the group consisting of electron transport complex protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating one or more activities selected from the group consisting of protease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamate, which comprises
(a) increasing or generating one or more activities selected from the group consisting of b1898-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating one or more activities selected from the group consisting of transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of uridine/cytidine kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating one or more activities selected from the group consisting of b2107-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of b2121-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating one or more activities selected from the group consisting of ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of NADH dehydrogenase I chain I in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating one or more activities selected from the group consisting of b2360-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating one or more activities selected from the group consisting of b2399-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of arginine, which comprises
(a) increasing or generating one or more activities selected from the group consisting of cysteine synthase A in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating one or more activities selected from the group consisting of cysteine synthase A in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating one or more activities selected from the group consisting of ethanolamine utilization protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of b2474-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of b2513-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of b2513-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating one or more activities selected from the group consisting of 2,3-dihydroxy-2,3-dihydrophenylpropionatedehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating one or more activities selected from the group consisting of 2,3-dihydroxy-2,3-dihydrophenylpropionatedehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of b2548-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating one or more activities selected from the group consisting of b2613-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of b2613-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of b2673-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of b2673-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of b2812-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of b2812-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of b2812-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of b2846-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of b2909-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating one or more activities selected from the group consisting of b2909-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of ribosephosphate isomerase, constitutive in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamate, which comprises
(a) increasing or generating one or more activities selected from the group consisting of arginine exporter protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating one or more activities selected from the group consisting of b2936-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of L-asparaginase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of murein transglycosylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of b2999-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating one or more activities selected from the group consisting of glycoprotease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of b3121-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating one or more activities selected from the group consisting of b3121-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of b3151-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of acetyl CoA carboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating one or more activities selected from the group consisting of acetyl CoA carboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of arginine, which comprises
(a) increasing or generating one or more activities selected from the group consisting of methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating one or more activities selected from the group consisting of methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of b3346-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of b3410-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating one or more activities selected from the group consisting of B3427-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating one or more activities selected from the group consisting of b3509-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating one or more activities selected from the group consisting of valine-pyruvate transaminase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of threonine dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of threonine dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of phosphopantetheine adenylyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of ATP synthase subunit beta in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating one or more activities selected from the group consisting of enterobacterial common antigen polymerase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating one or more activities selected from the group consisting of enterobacterial common antigen polymerase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating one or more activities selected from the group consisting of DNA helicase II in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of b3814-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of b3817-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of glycerol dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating one or more activities selected from the group consisting of b3989-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of arginine, which comprises
(a) increasing or generating one or more activities selected from the group consisting of b4029-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating one or more activities selected from the group consisting of b4029-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of b4029-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of cation/acetate symporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of arginine, which comprises
(a) increasing or generating one or more activities selected from the group consisting of b4121-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating one or more activities selected from the group consisting of b4121-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of lysyl-tRNA synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of aspartase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of gluconate transport system permease 3 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating one or more activities selected from the group consisting of purine-nucleoside phosphorylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating one or more activities selected from the group consisting of purine-nucleoside phosphorylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of thioredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating one or more activities selected from the group consisting of GM02LC11114-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating one or more activities selected from the group consisting of histone H2A in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating one or more activities selected from the group consisting of histone H2A in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of GM02LC17485-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating one or more activities selected from the group consisting of RNA binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of GM02LC46-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of GM02LC5744-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of s_pp015018333r-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating one or more activities selected from the group consisting of s_pp015018333r-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of sll0064-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of N-acetyl-gamma-glutamyl-phosphate reductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamate, which comprises
(a) increasing or generating one or more activities selected from the group consisting of heat shock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of arginine, which comprises
(a) increasing or generating one or more activities selected from the group consisting of flavodoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating one or more activities selected from the group consisting of phosphopantothenoylcysteinesynthetase/decarboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of sll0254-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of sll0254-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of polyphosphate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of sll0354-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of urease subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of NAD(P)H-quinone oxidoreductase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating one or more activities selected from the group consisting of quinolinate synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of permease protein of phosphate ABC transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating one or more activities selected from the group consisting of permease protein of phosphate ABC transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of oxireductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating one or more activities selected from the group consisting of oxireductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of oxireductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating one or more activities selected from the group consisting of malate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating one or more activities selected from the group consisting of malate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of malate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of phosphoribosylaminoimidazole carboxylase catalytic subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of 3-deoxy-7-phosphoheptulonate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of glycogen synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of carbon dioxide concentrating mechanism protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of carbon dioxide concentrating mechanism protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of carbon dioxide concentrating mechanism protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of phosphoribosylformyl glycinamidine synthase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of coproporphyrinogen oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of glycogen (starch) synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of glycogen (starch) synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of fatty acid desaturase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating one or more activities selected from the group consisting of CTP synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamate, which comprises
(a) increasing or generating one or more activities selected from the group consisting of nitrate/nitrite transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamate, which comprises
(a) increasing or generating one or more activities selected from the group consisting of CDP-diacylglycerol-glycerol-3-phosphate 3-phosphatidyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of arginine, which comprises
(a) increasing or generating one or more activities selected from the group consisting of glutathione S-transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating one or more activities selected from the group consisting of glutathione S-transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of glutathione S-transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of exopolyphosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating one or more activities selected from the group consisting of 4-alpha-glucanotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of alanine dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of sll1761-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating one or more activities selected from the group consisting of sll1761-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of coproporphyrinogen III oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of coproporphyrinogen III oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of coproporphyrinogen III oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of cation-transporting ATPase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of cation-transporting ATPase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating one or more activities selected from the group consisting of glycosidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating one or more activities selected from the group consisting of glycosidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of phosphoribosylglycinamide formyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of bifunctional purine biosynthesis protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating one or more activities selected from the group consisting of slr0600-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of pyrroline carboxylate reductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of Glu/Leu/Phe/Val dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of arginine, which comprises
(a) increasing or generating one or more activities selected from the group consisting of geranylgeranyl pyrophosphate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating one or more activities selected from the group consisting of geranylgeranyl pyrophosphate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of geranylgeranyl pyrophosphate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of circadian clock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating one or more activities selected from the group consisting of circadian clock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of amine oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of dihydrolipoamide dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of arginine, which comprises
(a) increasing or generating one or more activities selected from the group consisting of gamma-glutamyltranspeptidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating one or more activities selected from the group consisting of gamma-glutamyltranspeptidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of arginine, which comprises
(a) increasing or generating one or more activities selected from the group consisting of arginine decarboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating one or more activities selected from the group consisting of CDP-diglyceride synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamate, which comprises
(a) increasing or generating one or more activities selected from the group consisting of carbohydrate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises (a) increasing or generating one or more activities selected from the group consisting of iron(III) dicitrate-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of Photosystem I reaction center subunit XI in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of photosystem II protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating one or more activities selected from the group consisting of photosystem II protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of glutamine amidotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of glycerol-3-phosphate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating one or more activities selected from the group consisting of phosphoadenosine phosphosulfate reductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of riboflavin biosynthesis protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamate, which comprises
(a) increasing or generating one or more activities selected from the group consisting of malonyl CoA-acyl carrier protein transacylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises (a) increasing or generating one or more activities selected from the group consisting of malonyl CoA-acyl carrier protein transacylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of short-chain alcohol dehydrogenase family in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of Sec-independent protein translocase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of Sec-independent protein translocase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of cell division protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of branched-chain amino acid ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of amino acid ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of TTC0768-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of metal-dependent hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating one or more activities selected from the group consisting of multiple antibiotic resistance protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamate, which comprises
(a) increasing or generating one or more activities selected from the group consisting of TTC1386-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of homocitrate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of homocitrate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of oxidoreductase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of oxidoreductase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating one or more activities selected from the group consisting of XM_{—}473199-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of

XM_{—}473199-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism. Accordingly, the present invention relates to a process for the production of proline, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of cell division control protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating one or more activities selected from the group consisting of cell division control protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of YCL026C-A-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of 102c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of coproporphyrinogen III oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of branched-chain amino acid permease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of peroxisome assembly protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of arginine, which comprises
(a) increasing or generating one or more activities selected from the group consisting of ydr338c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating one or more activities selected from the group consisting of yer014w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating one or more activities selected from the group consisting of yer106w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of molecular chaperone portein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of YFLO19C-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of yfl054c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating one or more activities selected from the group consisting of yfl054c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating one or more activities selected from the group consisting of pre-mRNA-splicing factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating one or more activities selected from the group consisting of ygI237c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of ygr068c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of squalene monooxygenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of ygr221 c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of ygr221 c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of yhl013c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of mitochondrial processing protease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating one or more activities selected from the group consisting of H/ACA ribonucleoprotein complex subunit 3 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of yhr207c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of arginine, which comprises
(a) increasing or generating one or more activities selected from the group consisting of 3-phosphoglycerate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating one or more activities selected from the group consisting of allantoinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of allantoicase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating one or more activities selected from the group consisting of DnaJ-like chaperone in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating one or more activities selected from the group consisting of ornithine carbamoyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating one or more activities selected from the group consisting of potassium transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of glycogenin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating one or more activities selected from the group consisting of ATP-dependent RNA helicase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of phosphoribosyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating one or more activities selected from the group consisting of polygalacturonase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating one or more activities selected from the group consisting of aspartate aminotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating one or more activities selected from the group consisting of ylr065c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of ylr178c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of aconitate hydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of adenylosuccinate lyase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating one or more activities selected from the group consisting of ynl142w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of sterol O-acyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating one or more activities selected from the group consisting of glutaminyl -tRNA synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating one or more activities selected from the group consisting of glutaminyl-tRNA synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of yor221 c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of isopentenyl diphosphate isomerase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of arginine, which comprises
(a) increasing or generating one or more activities selected from the group consisting of Zm_{—}4842_BE510522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating one or more activities selected from the group consisting of Zm_{—}4842_BE510522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating one or more activities selected from the group consisting of Zm_{—}4842_BE510522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating one or more activities selected from the group consisting of phosphopantothenoylcysteine decarboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating one or more activities selected from the group consisting of ZM06LC11975-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

An embodiment of the present invention relates to a process for the production of arginine, which comprises
(a1) increasing or generating the activity of a polypeptide comprising a polypeptide as depicted in a respective line in column 5 or 8 of Table II, application no. 2, or a homolog or a fragment thereof, a consensus sequence or at least one polypeptide motif as depicted in the respective line in column 8 of Table IV, application no. 2, whereby the respective line discloses in column 7 arginine; or
(a2) increasing or generating the activity of an expression product of one or more nucleic acid molecule(s) comprising a polynucleotide as depicted in the respective line in column 5 or 8 of Table I, application no. 2, preferably the coding region thereof, or a homolog or a fragment thereof, whereby the respective line discloses in column 7 arginine; or
(a3) increasing or generating the activity of a functional equivalent of (a1) or (a2);
   in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of arginine or a composition comprising arginine in said non-human organism or in the culture medium surrounding said non-human organism.

An embodiment of the present invention relates to a process for the production of glutamate, which comprises
(a1) increasing or generating the activity of a polypeptide comprising a polypeptide as depicted in a respective line in column 5 or 8 of Table II, application no. 2, or a homolog or a fragment thereof, a consensus sequence or at least one polypeptide motif as depicted in the respective line in column 8 of Table IV, application no. 2, whereby the respective line discloses in column 7 glutamate; or
(a2) increasing or generating the activity of an expression product of one or more nucleic acid molecule(s) comprising a polynucleotide as depicted in the respective line in column 5 or 8 of Table I, application no. 2, preferably the coding region thereof, or a homolog or a fragment thereof, whereby the respective line discloses in column 7 glutamate; or
(a3) increasing or generating the activity of a functional equivalent of (a1) or (a2);
   in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of glutamate or a composition comprising glutamate in said non-human organism or in the culture medium surrounding said non-human organism.

An embodiment of the present invention relates to a process for the production of glutamine, which comprises
(a1) increasing or generating the activity of a polypeptide comprising a polypeptide as depicted in a respective line in column 5 or 8 of Table II, application no. 2, or a homolog or a fragment thereof, a consensus sequence or at least one polypeptide motif as depicted in the respective line in column 8 of Table IV, application no. 2, whereby the respective line discloses in column 7 glutamine; or
(a2) increasing or generating the activity of an expression product of one or more nucleic acid molecule(s) comprising a polynucleotide as depicted in the respective line in column 5 or 8 of Table I, application no. 2, preferably the coding region thereof, or a homolog or a fragment thereof, whereby the respective line discloses in column 7 glutamine; or
(a3) increasing or generating the activity of a functional equivalent of (a1) or (a2);
   in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of glutamine or a composition comprising glutamine in said non-human organism or in the culture medium surrounding said non-human organism.

An embodiment of the present invention relates to a process for the production of proline, which comprises
(a1) increasing or generating the activity of a polypeptide comprising a polypeptide as depicted in a respective line in column 5 or 8 of Table II, application no. 2, or a homolog or a fragment thereof, a consensus sequence or at least one polypeptide motif as depicted in the respective line in column 8 of Table IV, application no. 2, whereby the respective line discloses in column 7 proline; or
(a2) increasing or generating the activity of an expression product of one or more nucleic acid molecule(s) comprising a polynucleotide as depicted in the respective line in column 5 or 8 of Table I, application no. 2, preferably the coding region thereof, or a homolog or a fragment thereof, whereby the respective line discloses in column 7 proline; or
(a3) increasing or generating the activity of a functional equivalent of (a1) or (a2);
   in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of proline or a composition comprising proline in said non-human organism or in the culture medium surrounding said non-human organism.

Accordingly, the increase or generation of one or more said activities is for example conferred by one or more expression products of said nucleic acid molecule, e.g. proteins. Accordingly, in the present invention described above, the increase or generation of one or more said activities is for example conferred by one or more protein(s) each comprising a polypeptide selected from the group as depicted in Table II, application no. 2, column 5 and 8, or a homolog or a fragment thereof.

The process of the invention comprises in one embodiment the following steps:
(i) increasing or generating of the expression of; and/or
(ii) increasing or generating the expression of an expression product of; and/or
(iii) increasing or generating one or more activities of an expression product encoded by;
   at least one nucleic acid molecule (in the following “Fine Chemical Related Protein (FCRP)”encoding gene or “FCRP”-gene) comprising a nucleic acid molecule selected from the group consisting of:
(a) a nucleic acid molecule encoding the polypeptide shown in column 5 or 8 of Table II, preferably Table II B, application no. 2, or a homolog or a fragment thereof;
(b) a nucleic acid molecule shown in column 5 or 8 of Table I, preferably Table I B, application no. 2, or a homolog or a fragment thereof (preferably the coding region thereof);
(c) a nucleic acid molecule, which, as a result of the degeneracy of the genetic code, can be derived from a polypeptide sequence depicted in column 5 or 8 of Table II, preferably Table II B, application no. 2;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 2, or the coding region thereof;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d),
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 2;
(i) a nucleic acid molecule encoding a polypeptide having the activity represented by a respective protein as depicted in column 5 of Table II, application no. 2;
(j) a nucleic acid molecule which comprises a polynucleotide, which is obtained by amplifying a cDNA library or a genomic library using the primers as depicted in column 8 of Table III, application no. 2; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
   or a nucleic acid molecule comprising a sequence which is complementary thereto.
   (All the references to Tables I to IV are references to the corresponding lines of Tables I to IV.)

In a preferred embodiment thereof said nucleic acid molecule encodes a polypeptide which has the activity of the polypeptide represented by a protein comprising a polypeptide as depicted in the corresponding hit in column 5 of Table II, application no. 2.

In another preferred embodiment thereof said nucleic acid molecule confers a generation or an increase of the respective fine chemical in a non-human organism or a part thereof as compared to a corresponding wild-type non-human organism or part thereof.

In another preferred embodiment thereof said nucleic acid molecule encodes a polypeptide which has the activity of the polypeptide represented by a protein as depicted in the corresponding hit in column 5 of Table II, application no. 2, and confers a generation or an increase of the respective fine chemical in a non-human organism or a part thereof as compared to a corresponding wild-type non-human organism or part thereof.

Accordingly, the genes of the present invention or used in accordance with the present invention, which respectively encode a protein having an activity of 2,3-dihydroxy-2,3-dihydrophenylpropionatedehydrogenase, 2-oxoglutarate dehydrogenase E1 subunit, 30S ribosomal protein, 3-deoxy-7-phosphoheptulonate synthase, 3-phosphoglycerate dehydrogenase, 47266012-protein, 49747384_SOYBEAN-protein, 4-alpha-glucanotransferase, ABC transporter permease protein, acetyl CoA carboxylase, acetyltransferase, acid shock protein, aconitate hydratase, acyl-CoA dehydrogenase, acyl-CoA synthase, acyltransferase, adenylosuccinate lyase, adenylylsulfate kinase, alanine dehydrogenase, aldehyde dehydrogenase, allantoicase, allantoinase, amine oxidase, amino acid ABC transporter permease protein, amino acid acetyltransferase, aminotransferase, ankyrin repeat family protein, anthranilate synthase component II, arginine decarboxylase, arginine exporter protein, asparaginase, aspartase, aspartate aminotransferase, AT1g17440-protein, AO g19800-protein, Atl g29350-protein, AO g47380-protein, At1g67340-protein, At4g32480-protein, At5g16650-protein, ATP synthase subunit beta, ATP-binding component of a transport system, ATP-dependent RNA helicase, AX653549-protein, AY087308-protein, b0456-protein, b0518-protein, b1003-protein, b1024-protein, b1108-protein, b1137-protein, b1163-protein, b1259-protein, b1280-protein, b1330-protein, b1445-protein, b1522-protein, b1898-protein, b2107-protein, b2121-protein, b2360-protein, b2399-protein, b2474-protein, b2513-protein, b2548-protein, b2613-protein, b2673-protein, b2812-protein, b2846-protein, b2909-protein, b2936-protein, b2999-protein, b3121-protein, b3151-protein, b3346-protein, b3410-protein, B3427-protein, b3509-protein, b3814-protein, b3817-protein, b3989-protein, b4029-protein, b4121-protein, beta-galactosidase, beta-hydroxylase, bifunctional purine biosynthesis protein, branched-chain amino acid ABC transporter permease protein, branched-chain amino acid permease, calcium-dependent protein kinase, carbohydrate kinase, carbon dioxide concentrating mechanism protein, cation/acetate symporter, cation-transporting ATPase, CBL-interacting protein kinase, CCAAT-binding transcription factor, CDP-diacylglycerol-glycerol-3-phosphate 3-phosphatidyltransferase, CDP-diglyceride synthetase, cell division control protein, cell division protein, Chaperone protein CIpB, circadian clock protein, coproporphyrinogen III oxidase, coproporphyrinogen oxidase, CTP synthase, CTP synthetase, cullin, cyclin D, cysteine synthase A, delta-1-pyrroline 5-carboxylase synthetase, dihydrolipoamide acetyltransferase, dihydrolipoamide dehydrogenase, DNA binding protein, DNA helicase II, DNA helicase IV, DNA mismatch repair protein, DnaJ-like chaperone, electron transport complex protein, elongation factor Tu, enoyl-CoA hydratase, enterobacterial common antigen polymerase, ethanolamine utilization protein, eukaryotic translation initiation factor 5, exopolyphosphatase, fatty acid desaturase, flavodoxin, fumarylacetoacetate hydrolase, gammaglutamyltranspeptidase, geranylgeranyl pyrophosphate synthase, gibberellin 20-oxidase, Glu/Leu/PheNal dehydrogenase, gluconate transport system permease 3, glucose dehydrogenase, glucose-1-phosphate cytidylyltransferase, glucose-6-phosphate 1-dehydrogenase, glutamate-ammonia-ligase, glutamine amidotransferase, glutamine synthetase, glutaminyl-tRNA synthetase, glutaredoxin, glutathione S-transferase, glycerol dehydrogenase, glycerol-3-phosphate dehydrogenase, glycogen (starch) synthase, glycogen synthase, glycogenin, glycoprotease, glycosidase, glycosyl transferase, GM02LC11114-protein, GM02LC17485-protein, GM02LC46-protein, GM02LC5744-protein, H/ACA ribonucleoprotein complex subunit 3, harpin-induced family protein, heat shock protein, heat shock transcription factor, HesB/YadR/YfhF family protein, histone H2A, homocitrate synthase, hydrolase, integral membrane transporter family protein, iron(III) dicitrate-binding protein, isochorismate synthase, isocitrate dehydrogenase, isopentenyl diphosphate isomerase, L-asparaginase, L-aspartate oxidase, Leucyl/phenylalanyl-tRNA-protein transferase, lipoprotein precursor, L-ribulose-5-phosphate 4-epimerase, lysyl-tRNA synthetase, major facilitator superfamily transporter protein, malate dehydrogenase, malic enzyme, malonyl CoA-acyl carrier protein transacylase, membrane protein, membrane transport protein, metal-dependent hydrolase, methylglyoxal synthase, methyltransferase, mitochondrial processing protease, molecular chaperone portein, monothiol glutaredoxin, monthiol glutaredoxin, multiple antibiotic resistance protein, murein transglycosylase, N-acetyl-gamma-glutamyl-phosphate reductase, NAD(P)H-quinone oxidoreductase subunit, NADH dehydrogenase I chain I, nitrate/nitrite transport protein, ornithine carbamoyltransferase, oxidoreductase, oxidoreductase subunit, oxireductase, permease protein of phosphate ABC transporter, peroxisome assembly protein, phosphatidylinositol 3- and 4-kinase family protein, phosphoadenosine phosphosulfate reductase, phosphoanhydride phosphorylase, phosphopantetheine adenylyltransferase, phosphopantothenoylcysteine decarboxylase, phosphopantothenoylcysteinesynthetase/decarboxylase, phosphoribosylaminoimidazole carboxylase catalytic subunit, phosphoribosylformyl glycinamidine synthase subunit, phosphoribosylglycinamide formyltransferase, phosphoribosyltransferase, Photosystem I reaction center subunit XI, photosystem II protein, polygalacturonase, polyphosphate kinase, potassium transport protein, pre-mRNA-splicing factor, protease, protein kinase, protein phosphatase, purine nucleoside phosphorylase, purine-nucleoside phosphorylase, pyrroline carboxylate reductase, pyruvate kinase, quinolinate synthetase, riboflavin biosynthesis protein, ribosephosphate isomerase, constitutive, RNA binding protein, s_pp015018333r-protein, sec-independent protein translocase, Sec-independent protein translocase subunit, serine protease, serine protease inhibitor, short-chain alcohol dehydrogenase family, sll0064-protein, sll0254-protein, sll0354-protein, sll1761-protein, slr0600-protein, sodium/proton antiporter, squalene monooxygenase, sterol O-acyltransferase, thioredoxin, thioredoxin family protein, threonine dehydrogenase, threonine synthase, transcription factor, transcriptional regulator, transcriptional regulator protein, translation initiation factor subunit, transport protein, TTC0768-protein, TTC1386-protein, urease subunit, uridine/cytidine kinase, valine-pyruvate transaminase, XM_{—}473199-protein, YCL026C-A-protein, ycr102c-protein, ydr338c-protein, yer014w-protein, yer106w-protein, YFL019C-protein, yfl054c-protein, yg1237c-protein, ygr068c-protein, ygr221c-protein, yhl013c-protein, yhr207c-protein, ylr065c-protein, ylr178c-protein, ynl142w-protein, yor221c-protein, zinc finger protein, zinc transporter, Zm_{—}4842_BE510522-protein, or ZMO6LC11975-protein, which respectively encode a protein comprising a polypeptide encoded by a nucleic acid sequence as shown in Table I, application no. 2, column 5 or 8, (preferably the coding region thereof), or a homolog or a fragment thereof, which respectively encode a protein comprising a polypeptide as depicted in Table II, application no. 2, column 5 or 8, or a homolg or a fragment thereof, and/or which respectively can be amplified with the primer set shown in Table III, application no. 2, column 8, are also referred to as “FCRP genes”.

Proteins or polypeptides encoded by “FCRP-genes” are referred to as “Fine Chemical Related Proteins” or “FCRP”. For the purposes of the description of the present invention, the respective protein having an activity of 2,3-dihydroxy-2,3-dihydrophenylpropionatedehydrogenase, 2-oxoglutarate dehydrogenase E1 subunit, 30S ribosomal protein, 3-deoxy-7-phosphoheptulonate synthase, 3-phosphoglycerate dehydrogenase, 47266012-protein, 49747384_SOYBEAN-protein, 4-alpha-glucanotransferase, ABC transporter permease protein, acetyl CoA carboxylase, acetyltransferase, acid shock protein, aconitate hydratase, acyl-CoA dehydrogenase, acyl-CoA synthase, acyltransferase, adenylosuccinate lyase, adenylylsulfate kinase, alanine dehydrogenase, aldehyde dehydrogenase, allantoicase, allantoinase, amine oxidase, amino acid ABC transporter permease protein, amino acid acetyltransferase, aminotransferase, ankyrin repeat family protein, anthranilate synthase component II, arginine decarboxylase, arginine exporter protein, asparaginase, aspartase, aspartate aminotransferase, At1g17440-protein, At1g19800-protein, Atl g29350-protein, At1g47380-protein, At1g67340-protein, At4g32480-protein, At5g16650-protein, ATP synthase subunit beta, ATP-binding component of a transport system, ATP-dependent RNA helicase, AX653549-protein, AY087308-protein, b0456-protein, b0518-protein, b1003-protein, b1024-protein, b1108-protein, b1137-protein, b1163-protein, b1259-protein, b1280-protein, b1330-protein, b1445-protein, b1522-protein, b1898-protein, b2107-protein, b2121-protein, b2360-protein, b2399-protein, b2474-protein, b2513-protein, b2548-protein, b2613-protein, b2673-protein, b2812-protein, b2846-protein, b2909-protein, b2936-protein, b2999-protein, b3121-protein, b3151-protein, b3346-protein, b3410-protein, B3427-protein, b3509-protein, b3814-protein, b3817-protein, b3989-protein, b4029-protein, b4121-protein, beta-galactosidase, beta-hydroxylase, bifunctional purine biosynthesis protein, branched-chain amino acid ABC transporter permease protein, branched-chain amino acid permease, calcium-dependent protein kinase, carbohydrate kinase, carbon dioxide concentrating mechanism protein, cation/acetate symporter, cation-transporting ATPase, CBL-interacting protein kinase, CCAAT-binding transcription factor, CDP-diacylglycerol-glycerol-3-phosphate 3-phosphatidyltransferase, CDP-diglyceride synthetase, cell division control protein, cell division protein, Chaperone protein CIpB, circadian clock protein, coproporphyrinogen III oxidase, coproporphyrinogen oxidase, CTP synthase, CTP synthetase, cullin, cyclin D, cysteine synthase A, delta-1-pyrroline 5-carboxylase synthetase, dihydrolipoamide acetyltransferase, dihydrolipoamide dehydrogenase, DNA binding protein, DNA helicase II, DNA helicase IV, DNA mismatch repair protein, DnaJ-like chaperone, electron transport complex protein, elongation factor Tu, enoyl-CoA hydratase, enterobacterial common antigen polymerase, ethanolamine utilization protein, eukaryotic translation initiation factor 5, exopolyphosphatase, fatty acid desaturase, flavodoxin, fumarylacetoacetate hydrolase, gamma-glutamyltranspeptidase, geranylgeranyl pyrophosphate synthase, gibberellin 20-oxidase, Glu/Leu/PheNal dehydrogenase, gluconate transport system permease 3, glucose dehydrogenase, glucose-1-phosphate cytidylyltransferase, glucose-6-phosphate 1-dehydrogenase, glutamate-ammonia-ligase, glutamine amidotransferase, glutamine synthetase, glutaminyl-tRNA synthetase, glutaredoxin, glutathione S-transferase, glycerol dehydrogenase, glycerol-3-phosphate dehydrogenase, glycogen (starch) synthase, glycogen synthase, glycogenin, glycoprotease, glycosidase, glycosyl transferase, GM02LC11114-protein, GM02LC17485-protein, GM02LC46-protein, GM02LC5744-protein, H/ACA ribonucleoprotein complex subunit 3, harpin-induced family protein, heat shock protein, heat shock transcription factor, HesB/YadR/YfhF family protein, histone H2A, homocitrate synthase, hydrolase, integral membrane transporter family protein, iron(III) dicitrate-binding protein, isochorismate synthase, isocitrate dehydrogenase, isopentenyl diphosphate isomerase, L-asparaginase, L-aspartate oxidase, Leucyl/phenylalanyl-tRNA-protein transferase, lipoprotein precursor, L-ribulose-5-phosphate 4epimerase, lysyl-tRNA synthetase, major facilitator superfamily transporter protein, malate dehydrogenase, malic enzyme, malonyl CoA-acyl carrier protein transacylase, membrane protein, membrane transport protein, metal-dependent hydrolase, methylglyoxal synthase, methyltransferase, mitochondrial processing protease, molecular chaperone portein, monothiol glutaredoxin, monthiol glutaredoxin, multiple antibiotic resistance protein, murein transglycosylase, N-acetyl-gamma-glutamyl-phosphate reductase, NAD(P)H-quinone oxidoreductase subunit, NADH dehydrogenase I chain I, nitrate/nitrite transport protein, ornithine carbamoyltransferase, oxidoreductase, oxidoreductase subunit, oxireductase, permease protein of phosphate ABC transporter, peroxisome assembly protein, phosphatidylinositol 3- and 4-kinase family protein, phosphoadenosine phosphosulfate reductase, phosphoanhydride phosphorylase, phosphopantetheine adenylyltransferase, phosphopantothenoylcysteine decarboxylase, phosphopantothenoylcysteinesynthetase/decarboxylase, phosphoribosylaminoimidazole carboxylase catalytic subunit, phosphoribosylformyl glycinamidine synthase subunit, phosphoribosylglycinamide formyltransferase, phosphoribosyltransferase, Photosystem I reaction center subunit XI, photosystem II protein, polygalacturonase, polyphosphate kinase, potassium transport protein, pre-mRNA-splicing factor, protease, protein kinase, protein phosphatase, purine nucleoside phosphorylase, purine-nucleoside phosphorylase, pyrroline carboxylate reductase, pyruvate kinase, quinolinate synthetase, riboflavin biosynthesis protein, ribosephosphate isomerase, constitutive, RNA binding protein, s_pp015018333r-protein, sec-independent protein translocase, Sec-independent protein translocase subunit, serine protease, serine protease inhibitor, short-chain alcohol dehydrogenase family, sll0064-protein, sll0254-protein, sll0354-protein, sll1761-protein, slr0600-protein, sodium/proton antiporter, squalene monooxygenase, sterol O-acyltransferase, thioredoxin, thioredoxin family protein, threonine dehydrogenase, threonine synthase, transcription factor, transcriptional regulator, transcriptional regulator protein, translation initiation factor subunit, transport protein, TTC0768-protein, TTC1386-protein, urease subunit, uridine/cytidine kinase, valine-pyruvate transaminase, XM_{—}473199-protein, YCL026C-A-protein, ycr102c-protein, ydr338c-protein, yer014w-protein, yer106w-protein, YFL019C-protein, yfl054c-protein, ygI237c-protein, ygr068c-protein, ygr221 c-protein, yhl013c-protein, yhr207c-protein, ylr065c-protein, ylr178c-protein, ynl142w-protein, yor221c-protein, zinc finger protein, zinc transporter, Zm_{—}4842_BE510522-protein, or ZM06LC11975-protein, the respective protein comprising a polypeptide encoded by one or more respective nucleic acid sequences as shown in Table I, application no. 2, column 5 or 8, (preferably the coding region thereof), or a homolog or fragment thereof, the respective protein comprising a respective polypeptide as depicted in Table II, application no. 2, column 5 or 8, or a homolog or fragment thereof, the respective protein comprising a sequence corresponding to the consensus sequence as shown in Table IV, application no. 2, column 8, and/or the respective protein comprising at least one polypeptide motif as shown in Table IV, application no. 2, column 8 is also referred to as Fine Chemical Related Protein” or “FCRP”.

Thus, in one embodiment, the present invention provides a process of the production of arginine, glutamate, glutamine, and/or proline, by increasing or generating one or more activities, especially selected from the group consisting of 2,3-dihydroxy-2,3-dihydrophenylpropionatedehydrogenase, 2-oxoglutarate dehydrogenase E1 subunit, 30S ribosomal protein, 3-deoxy-7-phosphoheptulonate synthase, 3-phosphoglycerate dehydrogenase, 47266012-protein, 49747384_SOYBEAN-protein, 4-alpha-glucanotransferase, ABC transporter permease protein, acetyl CoA carboxylase, acetyltransferase, acid shock protein, aconitate hydratase, acyl-CoA dehydrogenase, acyl-CoA synthase, acyltransferase, adenylosuccinate lyase, adenylylsulfate kinase, alanine dehydrogenase, aldehyde dehydrogenase, allantoicase, allantoinase, amine oxidase, amino acid ABC transporter permease protein, amino acid acetyltransferase, aminotransferase, ankyrin repeat family protein, anthranilate synthase component II, arginine decarboxylase, arginine exporter protein, asparaginase, aspartase, aspartate aminotransferase, At1g17440-protein, At1g19800-protein, At1g29350-protein, At1g47380-protein, At1g67340-protein, At4g32480-protein, At5g16650-protein, ATP synthase subunit beta, ATP-binding component of a transport system, ATP-dependent RNA helicase, AX653549-protein, AY087308-protein, b0456-protein, b0518-protein, b1003-protein, b1024-protein, b1108-protein, b1137-protein, b1163-protein, b1259-protein, b1280-protein, b1330-protein, b1445-protein, b1522-protein, b1898-protein, b2107-protein, b2121-protein, b2360-protein, b2399-protein, b2474-protein, b2513-protein, b2548-protein, b2613-protein, b2673-protein, b2812-protein, b2846-protein, b2909-protein, b2936-protein, b2999-protein, b3121-protein, b3151-protein, b3346-protein, b3410-protein, B3427-protein, b3509-protein, b3814-protein, b3817-protein, b3989-protein, b4029-protein, b4121-protein, beta-galactosidase, beta-hydroxylase, bifunctional purine biosynthesis protein, branched-chain amino acid ABC transporter permease protein, branched-chain amino acid permease, calcium-dependent protein kinase, carbohydrate kinase, carbon dioxide concentrating mechanism protein, cation/acetate symporter, cation-transporting ATPase, CBL-interacting protein kinase, CCAAT-binding transcription factor, CDP-diacylglycerol-glycerol-3-phosphate 3-phosphatidyltransferase, CDP-diglyceride synthetase, cell division control protein, cell division protein, Chaperone protein CIpB, circadian clock protein, coproporphyrinogen III oxidase, coproporphyrinogen oxidase, CTP synthase, CTP synthetase, cullin, cyclin D, cysteine synthase A, delta-1-pyrroline 5-carboxylase synthetase, dihydrolipoamide acetyltransferase, dihydrolipoamide dehydrogenase, DNA binding protein, DNA helicase II, DNA helicase IV, DNA mismatch repair protein, DnaJ-like chaperone, electron transport complex protein, elongation factor Tu, enoyl-CoA hydratase, enterobacterial common antigen polymerase, ethanolamine utilization protein, eukaryotic translation initiation factor 5, exopolyphosphatase, fatty acid desaturase, flavodoxin, fumarylacetoacetate hydrolase, gamma-glutamyltranspeptidase, geranylgeranyl pyrophosphate synthase, gibberellin 20-oxidase, Glu/Leu/PheNal dehydrogenase, gluconate transport system permease 3, glucose dehydrogenase, glucose-1-phosphate cytidylyltransferase, glucose-6-phosphate 1-dehydrogenase, glutamate-ammonia-ligase, glutamine amidotransferase, glutamine synthetase, glutaminyl-tRNA synthetase, glutaredoxin, glutathione S-transferase, glycerol dehydrogenase, glycerol-3-phosphate dehydrogenase, glycogen (starch) synthase, glycogen synthase, glycogenin, glycoprotease, glycosidase, glycosyl transferase, GM02LC11114-protein, GM02LC17485-protein, GM02LC46-protein, GM02LC5744-protein, H/ACA ribonucleoprotein complex subunit 3, harpin-induced family protein, heat shock protein, heat shock transcription factor, HesB/YadR/YfhF family protein, histone H2A, homocitrate synthase, hydrolase, integral membrane transporter family protein, iron(III) dicitrate-binding protein, isochorismate synthase, isocitrate dehydrogenase, isopentenyl diphosphate isomerase, L-asparaginase, L-aspartate oxidase, Leucyl/phenylalanyl-tRNA-protein transferase, lipoprotein precursor, L-ribulose-5-phosphate 4-epimerase, lysyl-tRNA synthetase, major facilitator superfamily transporter protein, malate dehydrogenase, malic enzyme, malonyl CoA-acyl carrier protein transacylase, membrane protein, membrane transport protein, metal-dependent hydrolase, methylglyoxal synthase, methyltransferase, mitochondrial processing protease, molecular chaperone portein, monothiol glutaredoxin, monthiol glutaredoxin, multiple antibiotic resistance protein, murein transglycosylase, N-acetyl-gamma-glutamyl-phosphate reductase, NAD(P)H-quinone oxidoreductase subunit, NADH dehydrogenase I chain I, nitrate/nitrite transport protein, ornithine carbamoyltransferase, oxidoreductase, oxidoreductase subunit, oxireductase, permease protein of phosphate ABC transporter, peroxisome assembly protein, phosphatidylinositol 3- and 4-kinase family protein, phosphoadenosine phosphosulfate reductase, phosphoanhydride phosphorylase, phosphopantetheine adenylyltransferase, phosphopantothenoylcysteine decarboxylase, phosphopantothenoylcysteinesynthetase/decarboxylase, phosphoribosylaminoimidazole carboxylase catalytic subunit, phosphoribosylformyl glycinamidine synthase subunit, phosphoribosylglycinamide formyltransferase, phosphoribosyltransferase, Photosystem I reaction center subunit XI, photosystem II protein, polygalacturonase, polyphosphate kinase, potassium transport protein, pre-mRNA-splicing factor, protease, protein kinase, protein phosphatase, purine nucleoside phosphorylase, purine-nucleoside phosphorylase, pyrroline carboxylate reductase, pyruvate kinase, quinolinate synthetase, riboflavin biosynthesis protein, ribosephosphate isomerase, constitutive, RNA binding protein, s_pp015018333r-protein, sec-independent protein translocase, Sec-independent protein translocase subunit, serine protease, serine protease inhibitor, short-chain alcohol dehydrogenase family, sll0064-protein, sll0254-protein, sll0354-protein, sll1761-protein, slr0600-protein, sodium/proton antiporter, squalene monooxygenase, sterol O-acyltransferase, thioredoxin, thioredoxin family protein, threonine dehydrogenase, threonine synthase, transcription factor, transcriptional regulator, transcriptional regulator protein, translation initiation factor subunit, transport protein, TTC0768-protein, TTC1386-protein, urease subunit, uridine/cytidine kinase, valine-pyruvate transaminase, XM_{—}473199-protein, YCL026C-A-protein, ycr102c-protein, ydr338c-protein, yer014w-protein, yer106w-protein, YFL019C-protein, yfl054c-protein, ygI237c-protein, ygr068c-protein, ygr221 c-protein, yhl013c-protein, yhr207c-protein, ylr065c-protein, ylr178c-protein, ynl142w-protein, yor221c-protein, zinc finger protein, zinc transporter, Zm_{—}4842_BE510522-protein, and ZM06LC11975-protein, which is conferred by one or more FCRPs or the gene product of one or more FCRP-genes, for example by the gene product of a nucleic acid sequences comprising a polynucleotide selected from the group as shown in Table 1, application no. 2, column 5 or 8, (preferably by the coding region thereof), or a homolog or a fragment thereof, e.g. or by one or more proteins each comprising a polypeptide encoded by one or more nucleic acid sequences selected from the group as shown in Table I, application no. 2, column 5 or 8, (preferably by the coding region thereof), or a homolog or a fragment thereof, or by one or more protein(s) each comprising a polypeptide selected from the group as depicted in Table II, application no. 2, column 5 and 8, or a homolog thereof, or a protein comprising a sequence corresponding to the consensus sequence or comprising at least one polypeptide motif as shown in Table IV, application no. 2, column 8.

for the disclosure of this paragraph see [0025.1.1.1] above.

In an embodiment, the process comprises increasing or generating the activity of one or more polypeptides having said activity, e.g.

by generating or increasing the amount and/or specific activity in the cell or a compartment of a cell of one of more FCRP, especially selected from the group consisting of 2,3-dihydroxy-2,3-dihydrophenylpropionatedehydrogenase, 2-oxoglutarate dehydrogenase E1 subunit, 30S ribosomal protein, 3-deoxy-7-phosphoheptulonate synthase, 3-phosphoglycerate dehydrogenase, 47266012-protein, 49747384_SOYBEAN-protein, 4-alpha-glucanotransferase, ABC transporter permease protein, acetyl CoA carboxylase, acetyltransferase, acid shock protein, aconitate hydratase, acyl-CoA dehydrogenase, acyl-CoA synthase, acyltransferase, adenylosuccinate lyase, adenylylsulfate kinase, alanine dehydrogenase, aldehyde dehydrogenase, allantoicase, allantoinase, amine oxidase, amino acid ABC transporter permease protein, amino acid acetyltransferase, aminotransferase, ankyrin repeat family protein, anthranilate synthase component II, arginine decarboxylase, arginine exporter protein, asparaginase, aspartase, aspartate aminotransferase, At1g17440-protein, At1g19800-protein, At1g29350-protein, At1 g47380-protein, At1g67340-protein, At4g32480-protein, At5g16650-protein, ATP synthase subunit beta, ATP-binding component of a transport system, ATP-dependent RNA helicase, AX653549-protein, AY087308-protein, b0456-protein, b0518-protein, b1003-protein, b1024-protein, b1108-protein, b1137-protein, b1163-protein, b1259-protein, b1280-protein, b1330-protein, b1445-protein, b1522-protein, b1898-protein, b2107-protein, b2121-protein, b2360-protein, b2399-protein, b2474-protein, b2513-protein, b2548-protein, b2613-protein, b2673-protein, b2812-protein, b2846-protein, b2909-protein, b2936-protein, b2999-protein, b3121-protein, b3151-protein, b3346-protein, b3410-protein, B3427-protein, b3509-protein, b3814-protein, b3817-protein, b3989-protein, b4029-protein, b4121-protein, beta-galactosidase, beta-hydroxylase, bifunctional purine biosynthesis protein, branched-chain amino acid ABC transporter permease protein, branched-chain amino acid permease, calcium-dependent protein kinase, carbohydrate kinase, carbon dioxide concentrating mechanism protein, cation/acetate symporter, cation-transporting ATPase, CBL-interacting protein kinase, CCAAT-binding transcription factor, CDP-diacylglycerol-glycerol-3-phosphate 3-phosphatidyltransferase, CDP-diglyceride synthetase, cell division control protein, cell division protein, Chaperone protein CIpB, circadian clock protein, coproporphyrinogen III oxidase, coproporphyrinogen oxidase, CTP synthase, CTP synthetase, cullin, cyclin D, cysteine synthase A, delta-1-pyrroline 5-carboxylase synthetase, dihydrolipoamide acetyltransferase, dihydrolipoamide dehydrogenase, DNA binding protein, DNA helicase II, DNA helicase IV, DNA mismatch repair protein, DnaJ-like chaperone, electron transport complex protein, elongation factor Tu, enoyl-CoA hydratase, enterobacterial common antigen polymerase, ethanolamine utilization protein, eukaryotic translation initiation factor 5, exopolyphosphatase, fatty acid desaturase, flavodoxin, fumarylacetoacetate hydrolase, gamm-aglutamyltranspeptidase, geranylgeranyl pyrophosphate synthase, gibberellin 20-oxidase, Glu/Leu/PheNal dehydrogenase, gluconate transport system permease 3, glucose dehydrogenase, glucose-1-phosphate cytidylyltransferase, glucose-6-phosphate 1-dehydrogenase, glutamate-ammonia-ligase, glutamine amidotransferase, glutamine synthetase, glutaminyl-tRNA synthetase, glutaredoxin, glutathione S-transferase, glycerol dehydrogenase, glycerol-3-phosphate dehydrogenase, glycogen (starch) synthase, glycogen synthase, glycogenin, glycoprotease, glycosidase, glycosyl transferase, GM02LC11114-protein, GM02LC17485-protein, GM02LC46-protein, GM02LC5744-protein, H/ACA ribonucleoprotein complex subunit 3, harpin-induced family protein, heat shock protein, heat shock transcription factor, HesB/YadR/YfhF family protein, histone H2A, homocitrate synthase, hydrolase, integral membrane transporter family protein, iron(III) dicitrate-binding protein, isochorismate synthase, isocitrate dehydrogenase, isopentenyl diphosphate isomerase, L-asparaginase, L-aspartate oxidase, Leucyl/phenylalanyl-tRNA-protein transferase, lipoprotein precursor, L-ribulose-5-phosphate 4-epimerase, lysyl-tRNA synthetase, major facilitator superfamily transporter protein, malate dehydrogenase, malic enzyme, malonyl CoA-acyl carrier protein transacylase, membrane protein, membrane transport protein, metal-dependent hydrolase, methylglyoxal synthase, methyltransferase, mitochondrial processing protease, molecular chaperone portein, monothiol glutaredoxin, monthiol glutaredoxin, multiple antibiotic resistance protein, murein transglycosylase, N-acetyl-gamma-glutamyl-phosphate reductase, NAD(P)H-quinone oxidoreductase subunit, NADH dehydrogenase I chain I, nitrate/nitrite transport protein, ornithine carbamoyltransferase, oxidoreductase, oxidoreductase subunit, oxireductase, permease protein of phosphate ABC transporter, peroxisome assembly protein, phosphatidylinositol 3- and 4-kinase family protein, phosphoadenosine phosphosulfate reductase, phosphoanhydride phosphorylase, phosphopantetheine adenylyltransferase, phosphopantothenoylcysteine decarboxylase, phosphopanto-thenoylcysteinesynthetase/decarboxylase, phosphoribosylaminoimidazole carboxylase catalytic subunit, phosphoribosylformyl glycinamidine synthase subunit, phosphoribosylglycinamide formyltransferase, phosphoribosyltransferase, Photosystem I reaction center subunit XI, photosyste m II protein, polygalacturonase, polyphosphate kinase, potassium transport protein, pre-mRNA-splicing factor, protease, protein kinase, protein phosphatase, purine nucleoside phosphorylase, purine-nucleoside phosphorylase, pyrroline carboxylate reductase, pyruvate kinase, quinolinate synthetase, riboflavin biosynthesis protein, ribosephosphate isomerase, constitutive, RNA binding protein, s_pp015018333r-protein, sec-independent protein translocase, Sec-independent protein translocase subunit, serine protease, serine protease inhibitor, short-chain alcohol dehydrogenase family, sll0064-protein, sll0254-protein, sll0354-protein, sll1761-protein, slr0600-protein, sodium/proton antiporter, squalene monooxygenase, sterol O-acyltransferase, thioredoxin, thioredoxin family protein, threonine dehydrogenase, threonine synthase, transcription factor, transcriptional regulator, transcriptional regulator protein, translation initiation factor subunit, transport protein, TTC0768-protein, TTC1386-protein, urease subunit, uridine/cytidine kinase, valine-pyruvate transaminase, XM_{—}473199-protein, YCL026C-A-protein, ycr102c-protein, ydr338c-protein, yer014w-protein, yer106w-protein, YFL019C-protein, yfl054c-protein, ygl237c-protein, ygr068c-protein, ygr221 c-protein, yhl013c-protein, yhr207c-protein, ylr065cprotein, ylr178c-protein, ynl142w-protein, yor221c-protein, zinc finger protein, zinc transporter, Zm_{—}4842_BE510522-protein, and ZMO6LC11975-protein, for example of the respective polypeptide as depicted in Table II, application no. 2, column 5 and 8, or a homolog or a fragment thereof, or the respective polypeptide comprising a sequence corresponding to the consensus sequences as shown in Table IV, application no. 2, column 8, or the respective polypeptide comprising at least one polypeptide motif as depicted in Table IV, application no. 2, column 8.

A further embodiment of the present invention relates to a process for the production of glutamate, which comprises
(a) increasing or generating the activiy of a 47266012-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a 47266012-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating the activiy of a 49747384_SOYBEAN-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamate, which comprises
(a) increasing or generating the activiy of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating the activiy of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating the activiy of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of arginine, which comprises
(a) increasing or generating the activiy of a glucose-6-phosphate 1-dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating the activiy of a glucose-6-phosphate 1-dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a amino acid acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating the activiy of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating the activiy of a At1g17440-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating the activiy of a At1g19800-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating the activiy of a cullin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating the activiy of a cullin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a At1g29350-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of arginine, which comprises
(a) increasing or generating the activiy of a eukaryotic translation initiation factor 5 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating the activiy of a eukaryotic translation initiation factor 5 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating the activiy of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating the activiy of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a At1g47380-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating the activiy of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating the activiy of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating the activiy of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a At1g67340-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating the activiy of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a serine protease inhibitor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a CBL-interacting protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a CBL-interacting protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating the activiy of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a CBL-interacting protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a delta-1-pyrroline 5-carboxylase synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a phosphatidylinositol 3- and 4-kinase family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating the activiy of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating the activiy of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating the activiy of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a ankyrin repeat family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of arginine, which comprises
(a) increasing or generating the activiy of a thioredoxin family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating the activiy of a thioredoxin family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a thioredoxin family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a thioredoxin family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a harpin-induced family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating the activiy of a harpin-induced family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a harpin-induced family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a DNA binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a DNA mismatch repair protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a CCAAT-binding transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a glucose-6-phosphate 1-dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a monthiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a monthiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating the activiy of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating the activiy of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating the activiy of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamate, which comprises
(a) increasing or generating the activiy of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a At4g32480-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating the activiy of a At4g32480-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a At4g32480-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of arginine, which comprises
(a) increasing or generating the activiy of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating the activiy of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating the activiy of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating the activiy of a gibberellin 20-oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a integral membrane transporter family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a integral membrane transporter family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating the activiy of a At5g16650-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating the activiy of a At5g16650-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a At5g16650-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating the activiy of a translation initiation factor subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating the activiy of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating the activiy of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating the activiy of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating the activiy of a zinc finger protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating the activiy of a zinc finger protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating the activiy of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamate, which comprises
(a) increasing or generating the activiy of a adenylylsulfate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a adenylylsulfate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating the activiy of a malic enzyme non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a malic enzyme non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a glycosyl transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a sec-independent protein translocase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.
A further embodiment of the present invention relates to a process for the production of arginine, which comprises
(a) increasing or generating the activiy of a beta-hydroxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of arginine, which comprises
(a) increasing or generating the activiy of a elongation factor Tu non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating the activiy of a elongation factor Tu non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a elongation factor Tu non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating the activiy of a ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a acyl-CoA synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating the activiy of a acyl-CoA synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a acyl-CoA synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a acyl-CoA synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a 2-oxoglutarate dehydrogenase E1 subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of arginine, which comprises
(a) increasing or generating the activiy of a enoyl-CoA hydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating the activiy of a enoyl-CoA hydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating the activiy of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a acyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating the activiy of a lysyl-tRNA synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating the activiy of a 30S ribosomal protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamate, which comprises
(a) increasing or generating the activiy of a CTP synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a CTP synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating the activiy of a glucose-1-phosphate cytidylyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a glucose-1-phosphate cytidylyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a glucose-1-phosphate cytidylyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a aminotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating the activiy of a hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a glutamate-ammonia-ligase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating the activiy of a glutamate-ammonia-ligase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a glutamate-ammonia-ligase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a fumarylacetoacetate hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a HesB/YadR/YfhF family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating the activiy of a HesB/YadR/YfhF family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a HesB/YadR/YfhF family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a L-aspartate oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating the activiy of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a Chaperone protein CIpB non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a purine nucleoside phosphorylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating the activiy of a purine nucleoside phosphorylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a purine nucleoside phosphorylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a transcriptional regulator protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a AX653549-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a AX653549-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating the activiy of a AY087308-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating the activiy of a threonine synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating the activiy of a threonine synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a threonine synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a L-ribulose-5-phosphate 4-epimerase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a dihydrolipoamide acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating the activiy of a glucose dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating the activiy of a glucose dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a glucose dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a serine protease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a acyl-CoA dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating the activiy of a acyl-CoA dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating the activiy of a beta-galactosidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a ATP-binding component of a transport system non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a b0456-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a membrane transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating the activiy of a membrane transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a b0518-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating the activiy of a isochorismate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating the activiy of a isochorismate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a isochorismate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a isochorismate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a zinc transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating the activiy of a asparaginase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating the activiy of a Leucyl/phenylalanyl-tRNA-protein transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a Leucyl/phenylalanyl-tRNA-protein transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a major facilitator superfamily transporter protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating the activiy of a major facilitator superfamily transporter protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a DNA helicase IV non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a methylglyoxal synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a phosphoanhydride phosphorylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a b1003-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a b1003-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a lipoprotein precursor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating the activiy of a b1024-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a b1108-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a b1108-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a isocitrate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a b1137-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating the activiy of a b1137-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating the activiy of a b1137-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a b1163-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a sodium/proton antiporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a membrane protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a membrane protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a b1259-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating the activiy of a b1259-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating the activiy of a anthranilate synthase component II non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating the activiy of a b1280-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating the activiy of a glutamine synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating the activiy of a aldehyde dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamate, which comprises
(a) increasing or generating the activiy of a b1330-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a b1330-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating the activiy of a lipoprotein precursor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a lipoprotein precursor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating the activiy of a b1445-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a b1445-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating the activiy of a b1522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a acid shock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating the activiy of a electron transport complex protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating the activiy of a protease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamate, which comprises
(a) increasing or generating the activiy of a b1898-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating the activiy of a transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a uridine/cytidine kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating the activiy of a b2107-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a b2121-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating the activiy of a ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a NADH dehydrogenase I chain I non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating the activiy of a b2360-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating the activiy of a b2399-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of arginine, which comprises
(a) increasing or generating the activiy of a cysteine synthase A non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating the activiy of a cysteine synthase A non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating the activiy of a ethanolamine utilization protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a b2474-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a b2513-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a b2513-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating the activiy of a 2,3-dihydroxy-2,3-dihydrophenylpropionatedehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating the activiy of a 2,3-dihydroxy-2,3-dihydrophenylpropionatedehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a b2548-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating the activiy of a b2613-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a b2613-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a b2673-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a b2673-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating the activiy of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a b2812-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a b2812-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a b2812-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a b2846-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a b2909-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating the activiy of a b2909-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a ribosephosphate isomerase, constitutive non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamate, which comprises
(a) increasing or generating the activiy of a arginine exporter protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating the activiy of a b2936-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a L-asparaginase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a murein transglycosylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a b2999-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating the activiy of a glycoprotease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a b3121-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating the activiy of a b3121-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a b3151-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a acetyl CoA carboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating the activiy of a acetyl CoA carboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of arginine, which comprises
(a) increasing or generating the activiy of a methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating the activiy of a methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a b3346-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a b3410-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating the activiy of a B3427-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating the activiy of a b3509-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating the activiy of a valine-pyruvate transaminase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a threonine dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a threonine dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a phosphopantetheine adenylyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a ATP synthase subunit beta non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating the activiy of a enterobacterial common antigen polymerase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating the activiy of a enterobacterial common antigen polymerase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating the activiy of a DNA helicase II non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a b3814-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a b3817-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a glycerol dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating the activiy of a b3989-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating the activiy of a acetyltransferase non-targeted in a non-human organism or a part thereof, preferably a microorganism, a plant cell, a plant or a part thereof, as compared to a corresponding non-transformed wild type non-human organism or a part thereof; and (b) growing the non-human organism or a part thereof under conditions which permit the production of glutamate or a composition comprising glutamate in said non-human organism or in the culture medium surrounding said non-human organism.

A further embodiment of the present invention relates to a process for the production of arginine, which comprises
(a) increasing or generating the activiy of a b4029-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating the activiy of a b4029-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a b4029-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a cation/acetate symporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of arginine, which comprises
(a) increasing or generating the activiy of a b4121-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating the activiy of a b4121-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a lysyl-tRNA synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a aspartase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating the activiy of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating the activiy of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a gluconate transport system permease 3 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating the activiy of a purine-nucleoside phosphorylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating the activiy of a purine-nucleoside phosphorylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a thioredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating the activiy of a GM02LC11114-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating the activiy of a histone H2A non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating the activiy of a histone H2A non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a GM02LC17485-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating the activiy of a RNA binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating the activiy of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a GM02LC46-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a GM02LC5744-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating the activiy of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a s_pp015018333r-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating the activiy of a s_pp015018333r-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism. A further embodiment of the present invention relates to a process for the production of proline, which comprises
(a) increasing or generating the activiy of a sll0064-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a N-acetyl-gamma-glutamyl-phosphate reductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamate, which comprises
(a) increasing or generating the activiy of a heat shock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of arginine, which comprises
(a) increasing or generating the activiy of a flavodoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating the activiy of a phosphopantothenoylcysteinesynthetase/decarboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a sll0254-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a sll0254-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a polyphosphate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a sll0354-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a urease subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a NAD(P)H-quinone oxidoreductase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating the activiy of a quinolinate synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a permease protein of phosphate ABC transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating the activiy of a permease protein of phosphate ABC transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a oxireductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating the activiy of a oxireductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a oxireductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating the activiy of a malate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating the activiy of a malate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a malate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a phosphoribosylaminoimidazole carboxylase catalytic subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a 3-deoxy-7-phosphoheptulonate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a glycogen synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a carbon dioxide concentrating mechanism protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a carbon dioxide concentrating mechanism protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a carbon dioxide concentrating mechanism protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a phosphoribosylformyl glycinamidine synthase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a coproporphyrinogen oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a glycogen (starch) synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a glycogen (starch) synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a fatty acid desaturase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating the activiy of a CTP synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamate, which comprises
(a) increasing or generating the activiy of a nitrate/nitrite transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamate, which comprises
(a) increasing or generating the activiy of a CDP-diacylglycerol-glycerol-3-phosphate 3-phosphatidyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of arginine, which comprises
(a) increasing or generating the activiy of a glutathione S-transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating the activiy of a glutathione S-transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a glutathione S-transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a exopolyphosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating the activiy of a 4-alpha-glucanotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a alanine dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a sll1761-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating the activiy of a sll1761-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a coproporphyrinogen III oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a coproporphyrinogen III oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a coproporphyrinogen III oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a cation-transporting ATPase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a cation-transporting ATPase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating the activiy of a glycosidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating the activiy of a glycosidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a phosphoribosylglycinamide formyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a bifunctional purine biosynthesis protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating the activiy of a slr0600-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a pyrroline carboxylate reductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a Glu/Leu/Phe/Val dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of arginine, which comprises
(a) increasing or generating the activiy of a geranylgeranyl pyrophosphate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating the activiy of a geranylgeranyl pyrophosphate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a geranylgeranyl pyrophosphate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a circadian clock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating the activiy of a circadian clock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a amine oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a dihydrolipoamide dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of arginine, which comprises
(a) increasing or generating the activiy of a gamma-glutamyltranspeptidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating the activiy of a gamma-glutamyltranspeptidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of arginine, which comprises
(a) increasing or generating the activiy of a arginine decarboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating the activiy of a CDP-diglyceride synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamate, which comprises
(a) increasing or generating the activiy of a carbohydrate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a iron(III) dicitrate-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a Photosystem I reaction center subunit XI non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a photosystem II protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating the activiy of a photosystem II protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a glutamine amidotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a glycerol-3-phosphate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating the activiy of a phosphoadenosine phosphosulfate reductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a riboflavin biosynthesis protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamate, which comprises
(a) increasing or generating the activiy of a malonyl CoA-acyl carrier protein transacylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a malonyl CoA-acyl carrier protein transacylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a short-chain alcohol dehydrogenase family non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a Sec-independent protein translocase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a Sec-independent protein translocase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a cell division protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a branched-chain amino acid ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a amino acid ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a TTC0768-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a metal-dependent hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating the activiy of a multiple antibiotic resistance protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamate, which comprises
(a) increasing or generating the activiy of a TTC1386-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a homocitrate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a homocitrate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a oxidoreductase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a oxidoreductase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating the activiy of a XM_{—}473199-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a XM_{—}473199-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a cell division control protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating the activiy of a cell division control protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a YCL026C-A-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a ycr102c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating the activiy of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a coproporphyrinogen Ill oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a branched-chain amino acid permease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a peroxisome assembly protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of arginine, which comprises
(a) increasing or generating the activiy of a ydr338c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating the activiy of a yer014w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating the activiy of a yer106w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a molecular chaperone portein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a YFL019C-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a yfl054c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating the activiy of a yfl054c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating the activiy of a pre-mRNA-splicing factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating the activiy of a ygl237c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a ygr068c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a squalene monooxygenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a ygr221c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a ygr221c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a yhl013c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a mitochondrial processing protease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating the activiy of a H/ACA ribonucleoprotein complex subunit 3 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a yhr207c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of arginine, which comprises
(a) increasing or generating the activiy of a 3-phosphoglycerate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating the activiy of a allantoinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a allantoicase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating the activiy of a DnaJ-like chaperone non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating the activiy of a ornithine carbamoyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating the activiy of a potassium transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a glycogenin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating the activiy of a ATP-dependent RNA helicase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a phosphoribosyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating the activiy of a polygalacturonase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating the activiy of a aspartate aminotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating the activiy of a ylr065c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a ylr178c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a aconitate hydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a adenylosuccinate lyase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating the activiy of a ynl142w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a sterol O-acyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating the activiy of a glutaminyl-tRNA synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating the activiy of a glutaminyl-tRNA synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a yor221c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating the activiy of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a isopentenyl diphosphate isomerase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of arginine, which comprises
(a) increasing or generating the activiy of a Zm_{—}4842_BE510522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating the activiy of a Zm_{—}4842_BE510522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating the activiy of a Zm_{—}4842_BE510522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activiy of a phosphopantothenoylcysteine decarboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating the activiy of a ZM06LC11975-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating the activity of a polypeptide comprising a polypeptide as depicted in the respective line in column 5 or 8 of Table II, application no. 2, or a homolog or a fragment thereof, a consensus sequence or at least one polypeptide motif as depicted in the respective line in column 8 of Table IV, application no. 2, whereby the respective line discloses in column 7 arginine; or
increasing or generating the activity of an expression product of one or more nucleic acid molecule(s) comprising a polynucleotide as depicted in the respective line in column 5 or 8 of Table I, application no. 2, preferably the coding region thereof, or a homolog or a fragment thereof, whereby the respective line discloses in column 7 arginine; non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of arginine, or a composition comprising arginine in said non-human organism or in the culture medium surrounding said non-human organism.

A further embodiment of the present invention relates to a process for the production of glutamate, which comprises
(a) increasing or generating the activity of a polypeptide comprising a polypeptide as depicted in the respective line in column 5 or 8 of Table II, application no. 2, or a homolog or a fragment thereof, a consensus sequence or at least one polypeptide motif as depicted in the respective line in column 8 of Table IV, application no. 2, whereby the respective line discloses in column 7 glutamate; or
increasing or generating the activity of an expression product of one or more nucleic acid molecule(s) comprising a polynucleotide as depicted in the respective line in column 5 or 8 of Table I, application no. 2, preferably the coding region thereof, or a homolog or a fragment thereof, whereby the respective line discloses in column 7 glutamate; non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of glutamate, or a composition comprising glutamate in said non-human organism or in the culture medium surrounding said non-human organism.

A further embodiment of the present invention relates to a process for the production of glutamine, which comprises
(a) increasing or generating the activity of a polypeptide comprising a polypeptide as depicted in the respective line in column 5 or 8 of Table II, application no. 2, or a homolog or a fragment thereof, a consensus sequence or at least one polypeptide motif as depicted in the respective line in column 8 of Table IV, application no. 2, whereby the respective line discloses in column 7 glutamine; or
increasing or generating the activity of an expression product of one or more nucleic acid molecule(s) comprising a polynucleotide as depicted in the respective line in column 5 or 8 of Table I, application no. 2, preferably the coding region thereof, or a homolog or a fragment thereof, whereby the respective line discloses in column 7 glutamine; non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of glutamine, or a composition comprising glutamine in said non-human organism or in the culture medium surrounding said non-human organism.

A further embodiment of the present invention relates to a process for the production of proline, which comprises
(a) increasing or generating the activity of a polypeptide comprising a polypeptide as depicted in the respective line in column 5 or 8 of Table II, application no. 2, or a homolog or a fragment thereof, a consensus sequence or at least one polypeptide motif as depicted in the respective line in column 8 of Table IV, application no. 2, whereby the respective line discloses in column 7 proline; or
increasing or generating the activity of an expression product of one or more nucleic acid molecule(s) comprising a polynucleotide as depicted in the respective line in column 5 or 8 of Table I, application no. 2, preferably the coding region thereof, or a homolog or a fragment thereof, whereby the respective line discloses in column 7 proline; non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of proline, or a composition comprising proline in said non-human organism or in the culture medium surrounding said non-human organism.

A further embodiment of the present invention relates to a process for the production of glutamate, which comprises
(a) increasing or generating one or more activities selected from the group consisting of 47266012-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of 47266012-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating one or more activities selected from the group consisting of 49747384_SOYBEAN-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamate, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of arginine, which comprises
(a) increasing or generating one or more activities selected from the group consisting of glucose-6-phosphate 1-dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating one or more activities selected from the group consisting of glucose-6-phosphate 1-dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of amino acid acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating one or more activities selected from the group consisting of At1g17440-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating one or more activities selected from the group consisting of At1g19800-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating one or more activities selected from the group consisting of cullin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating one or more activities selected from the group consisting of cullin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of At1g29350-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of arginine, which comprises
(a) increasing or generating one or more activities selected from the group consisting of eukaryotic translation initiation factor 5 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating one or more activities selected from the group consisting of eukaryotic translation initiation factor 5 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of At1g47380-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of At1g67340-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of serine protease inhibitor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of CBL-interacting protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of CBL-interacting protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of CBL-interacting protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of delta-1-pyrroline 5-carboxylase synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of phosphatidylinositol 3- and 4-kinase family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of ankyrin repeat family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of arginine, which comprises
(a) increasing or generating one or more activities selected from the group consisting of thioredoxin family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating one or more activities selected from the group consisting of thioredoxin family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of thioredoxin family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of thioredoxin family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of harpin-induced family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating one or more activities selected from the group consisting of harpin-induced family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of harpin-induced family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of DNA binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of DNA mismatch repair protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of CCAAT-binding transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of glucose-6-phosphate 1-dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of monthiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of monthiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamate, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of At4g32480-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating one or more activities selected from the group consisting of At4g32480-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of At4g32480-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of arginine, which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating one or more activities selected from the group consisting of gibberellin 20-oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of integral membrane transporter family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of integral membrane transporter family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating one or more activities selected from the group consisting of At5g16650-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating one or more activities selected from the group consisting of At5g16650-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of At5g16650-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating one or more activities selected from the group consisting of translation initiation factor subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating one or more activities selected from the group consisting of zinc finger protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating one or more activities selected from the group consisting of zinc finger protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamate, which comprises
(a) increasing or generating one or more activities selected from the group consisting of adenylylsulfate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of adenylylsulfate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating one or more activities selected from the group consisting of malic enzyme in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of malic enzyme in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of glycosyl transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of sec-independent protein translocase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of arginine, which comprises
(a) increasing or generating one or more activities selected from the group consisting of betahydroxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of arginine, which comprises
(a) increasing or generating one or more activities selected from the group consisting of elongation factor Tu in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating one or more activities selected from the group consisting of elongation factor Tu in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of elongation factor Tu in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating one or more activities selected from the group consisting of ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of acyl-CoA synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating one or more activities selected from the group consisting of acyl-CoA synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of acyl-CoA synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of acyl-CoA synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of 2-oxoglutarate dehydrogenase E1 subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of arginine, which comprises
(a) increasing or generating one or more activities selected from the group consisting of enoyl-CoA hydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating one or more activities selected from the group consisting of enoyl-CoA hydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of acyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating one or more activities selected from the group consisting of lysyl-tRNA synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating one or more activities selected from the group consisting of 30S ribosomal protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamate, which comprises
(a) increasing or generating one or more activities selected from the group consisting of CTP synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of CTP synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating one or more activities selected from the group consisting of glucose-1-phosphate cytidylyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of glucose-1-phosphate cytidylyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of glucose-1-phosphate cytidylyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of aminotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating one or more activities selected from the group consisting of hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of glutamate-ammonia-ligase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating one or more activities selected from the group consisting of glutamate-ammonia-ligase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of glutamate-ammonia-ligase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of fumarylacetoacetate hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of HesB/YadR/YfhF family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating one or more activities selected from the group consisting of HesB/YadR/YfhF family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of HesB/YadR/YfhF family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of L-aspartate oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of Chaperone protein CIpB in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of purine nucleoside phosphorylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating one or more activities selected from the group consisting of purine nucleoside phosphorylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of purine nucleoside phosphorylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of transcriptional regulator protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of AX653549-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of AX653549-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating one or more activities selected from the group consisting of AY087308-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating one or more activities selected from the group consisting of threonine synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating one or more activities selected from the group consisting of threonine synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of threonine synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of L-ribulose-5-phosphate 4-epimerase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of dihydrolipoamide acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating one or more activities selected from the group consisting of glucose dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating one or more activities selected from the group consisting of glucose dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of glucose dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of serine protease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of acyl-CoA dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating one or more activities selected from the group consisting of acyl-CoA dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating one or more activities selected from the group consisting of beta-galactosidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of ATP-binding component of a transport system in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of b0456-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of membrane transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating one or more activities selected from the group consisting of membrane transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of b0518-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating one or more activities selected from the group consisting of isochorismate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating one or more activities selected from the group consisting of isochorismate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of isochorismate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of isochorismate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of zinc transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating one or more activities selected from the group consisting of asparaginase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating one or more activities selected from the group consisting of Leucyl/phenylalanyl-tRNA-protein transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of Leucyl/phenylalanyl-tRNA-protein transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of major facilitator superfamily transporter protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating one or more activities selected from the group consisting of major facilitator superfamily transporter protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of DNA helicase IV in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of methylglyoxal synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of phosphoanhydride phosphorylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of b1003-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of b1003-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of lipoprotein precursor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating one or more activities selected from the group consisting of b1024-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of b1108-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of b1108-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of isocitrate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of b1137-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating one or more activities selected from the group consisting of b1137-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating one or more activities selected from the group consisting of b1137-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of b1163-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of sodium/proton antiporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of membrane protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of membrane protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of b1259-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating one or more activities selected from the group consisting of b1259-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating one or more activities selected from the group consisting of anthranilate synthase component II in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating one or more activities selected from the group consisting of b1280-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating one or more activities selected from the group consisting of glutamine synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating one or more activities selected from the group consisting of aldehyde dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamate, which comprises
(a) increasing or generating one or more activities selected from the group consisting of b1330-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of b1330-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating one or more activities selected from the group consisting of lipoprotein precursor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of lipoprotein precursor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating one or more activities selected from the group consisting of b1445-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of b1445-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating one or more activities selected from the group consisting of b1522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of acid shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating one or more activities selected from the group consisting of electron transport complex protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating one or more activities selected from the group consisting of protease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamate, which comprises
(a) increasing or generating one or more activities selected from the group consisting of b1898-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating one or more activities selected from the group consisting of transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of uridine/cytidine kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating one or more activities selected from the group consisting of b2107-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of b2121-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating one or more activities selected from the group consisting of ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of NADH dehydrogenase I chain I in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating one or more activities selected from the group consisting of b2360-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating one or more activities selected from the group consisting of b2399-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of arginine, which comprises
(a) increasing or generating one or more activities selected from the group consisting of cysteine synthase A in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating one or more activities selected from the group consisting of cysteine synthase A in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating one or more activities selected from the group consisting of ethanolamine utilization protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of b2474-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of b2513-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of b2513-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating one or more activities selected from the group consisting of 2,3-dihydroxy-2,3-dihydrophenylpropionatedehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating one or more activities selected from the group consisting of 2,3-dihydroxy-2,3-dihydrophenylpropionatedehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of b2548-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating one or more activities selected from the group consisting of b2613-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of b2613-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of b2673-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of b2673-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of b2812-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of b2812-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of b2812-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of b2846-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of b2909-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating one or more activities selected from the group consisting of b2909-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of ribosephosphate isomerase, constitutive in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamate, which comprises
(a) increasing or generating one or more activities selected from the group consisting of arginine exporter protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating one or more activities selected from the group consisting of b2936-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of L-asparaginase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of murein transglycosylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of b2999-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating one or more activities selected from the group consisting of glycoprotease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of b3121-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating one or more activities selected from the group consisting of b3121-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of b3151-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of acetyl CoA carboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating one or more activities selected from the group consisting of acetyl CoA carboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of arginine, which comprises
(a) increasing or generating one or more activities selected from the group consisting of methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating one or more activities selected from the group consisting of methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of b3346-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of b3410-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating one or more activities selected from the group consisting of B3427-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating one or more activities selected from the group consisting of b3509-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating one or more activities selected from the group consisting of valine-pyruvate transaminase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of threonine dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of threonine dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of phosphopantetheine adenylyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of ATP synthase subunit beta in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating one or more activities selected from the group consisting of enterobacterial common antigen polymerase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating one or more activities selected from the group consisting of enterobacterial common antigen polymerase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating one or more activities selected from the group consisting of DNA helicase II in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of b3814-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of b3817-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of glycerol dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating one or more activities selected from the group consisting of b3989-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of arginine, which comprises
(a) increasing or generating one or more activities selected from the group consisting of b4029-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating one or more activities selected from the group consisting of b4029-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of b4029-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of cation/acetate symporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of arginine, which comprises
(a) increasing or generating one or more activities selected from the group consisting of b4121-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating one or more activities selected from the group consisting of b4121-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of lysyltRNA synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of aspartase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of gluconate transport system permease 3 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating one or more activities selected from the group consisting of purine-nucleoside phosphorylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating one or more activities selected from the group consisting of purine-nucleoside phosphorylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of thioredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating one or more activities selected from the group consisting of GM02LC11114-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating one or more activities selected from the group consisting of histone H2A in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating one or more activities selected from the group consisting of histone H2A in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of GM02LC17485-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating one or more activities selected from the group consisting of RNA binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of GM02LC46-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of GM02LC5744-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of s_pp015018333r-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating one or more activities selected from the group consisting of s_pp015018333r-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of sll0064-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of N-acetyl-gamma-glutamyl-phosphate reductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamate, which comprises
(a) increasing or generating one or more activities selected from the group consisting of heat shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of arginine, which comprises
(a) increasing or generating one or more activities selected from the group consisting of flavodoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating one or more activities selected from the group consisting of phosphopantothenoylcysteinesynthetase/decarboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of sll0254-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of sll0254-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of polyphosphate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of sll0354-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of urease subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of NAD(P)H-quinone oxidoreductase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating one or more activities selected from the group consisting of quinolinate synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of permease protein of phosphate ABC transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating one or more activities selected from the group consisting of permease protein of phosphate ABC transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of oxireductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating one or more activities selected from the group consisting of oxireductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of oxireductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating one or more activities selected from the group consisting of malate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating one or more activities selected from the group consisting of malate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of malate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of phosphoribosylaminoimidazole carboxylase catalytic subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of 3-deoxy-7-phosphoheptulonate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of glycogen synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of carbon dioxide concentrating mechanism protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of carbon dioxide concentrating mechanism protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of carbon dioxide concentrating mechanism protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of phosphoribosylformyl glycinamidine synthase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of coproporphyrinogen oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of glycogen (starch) synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of glycogen (starch) synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of fatty acid desaturase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating one or more activities selected from the group consisting of CTP synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamate, which comprises
(a) increasing or generating one or more activities selected from the group consisting of nitrate/nitrite transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamate, which comprises
(a) increasing or generating one or more activities selected from the group consisting of CDP-diacylglycerol--glycerol-3-phosphate 3-phosphatidyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of arginine, which comprises
(a) increasing or generating one or more activities selected from the group consisting of glutathione S-transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating one or more activities selected from the group consisting of glutathione S-transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of glutathione S-transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of exopolyphosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating one or more activities selected from the group consisting of 4-alpha-glucanotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of alanine dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of sll1761-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating one or more activities selected from the group consisting of sll1761-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of coproporphyrinogen III oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of coproporphyrinogen III oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of coproporphyrinogen III oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of cation-transporting ATPase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of cation-transporting ATPase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating one or more activities selected from the group consisting of glycosidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating one or more activities selected from the group consisting of glycosidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of phosphoribosylglycinamide formyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of bifunctional purine biosynthesis protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating one or more activities selected from the group consisting of slr0600-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of pyrroline carboxylate reductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of Glu/Leu/Phe/Val dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of arginine, which comprises
(a) increasing or generating one or more activities selected from the group consisting of geranylgeranyl pyrophosphate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating one or more activities selected from the group consisting of geranylgeranyl pyrophosphate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of geranylgeranyl pyrophosphate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of circadian clock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating one or more activities selected from the group consisting of circadian clock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of amine oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of dihydrolipoamide dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of arginine, which comprises
(a) increasing or generating one or more activities selected from the group consisting of gamma-glutamyltranspeptidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating one or more activities selected from the group consisting of gamma-glutamyltranspeptidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of arginine, which comprises
(a) increasing or generating one or more activities selected from the group consisting of arginine decarboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating one or more activities selected from the group consisting of CDP-diglyceride synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamate, which comprises
(a) increasing or generating one or more activities selected from the group consisting of carbohydrate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of iron(III) dicitrate-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of Photosystem I reaction center subunit XI in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of photosystem II protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating one or more activities selected from the group consisting of photosystem II protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of glutamine amidotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of glycerol-3-phosphate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating one or more activities selected from the group consisting of phosphoadenosine phosphosulfate reductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of riboflavin biosynthesis protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamate, which comprises
(a) increasing or generating one or more activities selected from the group consisting of malonyl CoA-acyl carrier protein transacylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of malonyl CoA-acyl carrier protein transacylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of short-chain alcohol dehydrogenase family in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of Sec-independent protein translocase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of Sec-independent protein translocase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of cell division protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of branched-chain amino acid ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of amino acid ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of TTC0768-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of metal-dependent hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating one or more activities selected from the group consisting of multiple antibiotic resistance protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamate, which comprises
(a) increasing or generating one or more activities selected from the group consisting of TTC1386-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of homocitrate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of homocitrate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of oxidoreductase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of oxidoreductase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating one or more activities selected from the group consisting of XM_{—}473199-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of XM_{—}473199-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of cell division control protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating one or more activities selected from the group consisting of cell division control protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of YCL026C-A-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of ycr102c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of coproporphyrinogen Ill oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of branched-chain amino acid permease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of peroxisome assembly protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of arginine, which comprises
(a) increasing or generating one or more activities selected from the group consisting of ydr338c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating one or more activities selected from the group consisting of yer014w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamine, which comprises
(a) increasing or generating one or more activities selected from the group consisting of yer106w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of molecular chaperone portein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of YFL019C-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of yf1054c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating one or more activities selected from the group consisting of yfl054c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating one or more activities selected from the group consisting of pre-mRNA-splicing factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating one or more activities selected from the group consisting of ygl237c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of ygr068c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of squalene monooxygenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of ygr221c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of ygr221c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of yhl013c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of mitochondrial processing protease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating one or more activities selected from the group consisting of H/ACA ribonucleoprotein complex subunit 3 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of yhr207c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of arginine, which comprises
(a) increasing or generating one or more activities selected from the group consisting of 3-phosphoglycerate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating one or more activities selected from the group consisting of allantoinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of allantoicase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating one or more activities selected from the group consisting of DnaJ-like chaperone in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of arginine, which comprises
(a) increasing or generating one or more activities selected from the group consisting of ornithine carbamoyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating one or more activities selected from the group consisting of potassium transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of glycogenin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating one or more activities selected from the group consisting of ATP-dependent RNA helicase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of phosphoribosyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of glutamate, which comprises
(a) increasing or generating one or more activities selected from the group consisting of polygalacturonase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of proline, which comprises
(a) increasing or generating one or more activities selected from the group consisting of aspartate aminotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamate, which comprises
(a) increasing or generating one or more activities selected from the group consisting of ylr065c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of ylr178c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of aconitate hydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of adenylosuccinate lyase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of glutamine, which comprises
(a) increasing or generating one or more activities selected from the group consisting of ynl142w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of sterol O-acyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamate, which comprises
(a) increasing or generating one or more activities selected from the group consisting of glutaminyl-tRNA synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

A further embodiment of the present invention relates to a process for the production of glutamine, which comprises
(a) increasing or generating one or more activities selected from the group consisting of glutaminyl-tRNA synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of yor221c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of proline, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of isopentenyl diphosphate isomerase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of arginine, which comprises
(a) increasing or generating one or more activities selected from the group consisting of Zm_{—}4842_BE510522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of glutamine, which comprises
(a) increasing or generating one or more activities selected from the group consisting of Zm_{—}4842_BE510522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

A further embodiment of the present invention relates to a process for the production of proline, which comprises
(a) increasing or generating one or more activities selected from the group consisting of Zm_{—}4842_BE510522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a) increasing or generating one or more activities selected from the group consisting of phosphopantothenoylcysteine decarboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

A further embodiment of the present invention relates to a process for the production of proline, which comprises
(a) increasing or generating one or more activities selected from the group consisting of ZM06LC11975-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

A further embodiment of the present invention relates to a process for the production of arginine, which comprises
(a1) increasing or generating the activity of a polypeptide comprising a polypeptide as depicted in the respective line in column 5 or 8 of Table II, application no. 2, or a homolog or fragment thereof, a consensus sequence or at least one polypeptide motif as depicted in column 8 of Table IV, application no. 2; or
increasing or generating the activity of an expression product of one or more nucleic acid molecule(s) comprising a polynucleotide as depicted in the respective line in column 5 or 8 of Table I, application no. 2, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 2, or a homolog or a fragment thereof, a consensus sequence or at least one polypeptide motif as depicted in the respective line in column 8 of Table IV, application no. 2, which is joined to a transit peptide; or
increasing or generating the activity of an expression product of one or more nucleic acid molecule(s) comprising a polynucleotide as depicted in the respective line in column 5 or 8 of Table I, application no. 2, preferably the coding region thereof, or a homolog or a fragment thereof, which is joined to a nucleic acid sequence encoding an organelle localization sequence, preferably a plastid or a mitochondrion locatization sequence, especially a plastid localization sequence;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 2, or a homolog or a fragment thereof, a consensus sequence or at least one polypeptide motif as depicted in the respective line in column 8 of Table IV, application no. 2; or
increasing or generating the activity of an expression product of one or more nucleic acid molecule(s) comprising a polynucleotide as depicted in the respective line in column 5 or 8 of Table I, application no. 2, preferably the coding region thereof, or a homolog or a fragment thereof;
in an organelle, preferably in plastids or imitochondra, especially in plastids, in a non-human organism or a part thereof; preferably a microorganism, a plant cell, a plant or a part thereof, through transformation of the organelle, as compared to a corresponding non-transformed wild type non-human organism or a part thereof;
   (wherein in the respective line in the respective Table in column 7 arginine is depicted) and
(b) growing the non-human organism under conditions which permit the production of arginine, or a composition comprising arginine in said non-human organism or in the culture medium surrounding said non-human organism.

A further embodiment of the present invention relates to a process for the production of glutamate, which comprises
(a1) increasing or generating the activity of a polypeptide comprising a polypeptide as depicted in the respective line in column 5 or 8 of Table II, application no. 2, or a homolog or fragment thereof, a consensus sequence or at least one polypeptide motif as depicted in column 8 of Table IV, application no. 2; or
increasing or generating the activity of an expression product of one or more nucleic acid molecule(s) comprising a polynucleotide as depicted in the respective line in column 5 or 8 of Table I, application no. 2, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 2, or a homolog or a fragment thereof, a consensus sequence or at least one polypeptide motif as depicted in the respective line in column 8 of Table IV, application no. 2, which is joined to a transit peptide; or
increasing or generating the activity of an expression product of one or more nucleic acid molecule(s) comprising a polynucleotide as depicted in the respective line in column 5 or 8 of Table I, application no. 2, preferably the coding region thereof, or a homolog or a fragment thereof, which is joined to a nucleic acid sequence encoding an organelle localization sequence, preferably a plastid or a mitochondrion locatization sequence, especially a plastid localization sequence;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 2, or a homolog or a fragment thereof, a consensus sequence or at least one polypeptide motif as depicted in the respective line in column 8 of Table IV, application no. 2; or
increasing or generating the activity of an expression product of one or more nucleic acid molecule(s) comprising a polynucleotide as depicted in the respective line in column 5 or 8 of Table I, application no. 2, preferably the coding region thereof, or a homolog or a fragment thereof;
in an organelle, preferably in plastids or imitochondra, especially in plastids, in a non-human organism or a part thereof; preferably a microorganism, a plant cell, a plant or a part thereof, through transformation of the organelle, as compared to a corresponding non-transformed wild type non-human organism or a part thereof;
   (wherein in the respective line in the respective Table in column 7 glutamate is depicted) and
(b) growing the non-human organism under conditions which permit the production of glutamate, or a composition comprising glutamate in said non-human organism or in the culture medium surrounding said non-human organism.

A further embodiment of the present invention relates to a process for the production of glutamine, which comprises
(a1) increasing or generating the activity of a polypeptide comprising a polypeptide as depicted in the respective line in column 5 or 8 of Table II, application no. 2, or a homolog or fragment thereof, a consensus sequence or at least one polypeptide motif as depicted in column 8 of Table IV, application no. 2; or
increasing or generating the activity of an expression product of one or more nucleic acid molecule(s) comprising a polynucleotide as depicted in the respective line in column 5 or 8 of Table I, application no. 2, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 2, or a homolog or a fragment thereof, a consensus sequence or at least one polypeptide motif as depicted in the respective line in column 8 of Table IV, application no. 2, which is joined to a transit peptide; or
increasing or generating the activity of an expression product of one or more nucleic acid molecule(s) comprising a polynucleotide as depicted in the respective line in column 5 or 8 of Table I, application no. 2, preferably the coding region thereof, or a homolog or a fragment thereof, which is joined to a nucleic acid sequence encoding an organelle localization sequence, preferably a plastid or a mitochondrion locatization sequence, especially a plastid localization sequence;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 2, or a homolog or a fragment thereof, a consensus sequence or at least one polypeptide motif as depicted in the respective line in column 8 of Table IV, application no. 2; or increasing or generating the activity of an expression product of one or more nucleic acid molecule(s) comprising a polynucleotide as depicted in the respective line in column 5 or 8 of Table I, application no. 2, preferably the coding region thereof, or a homolog or a fragment thereof;
in an organelle, preferably in plastids or imitochondra, especially in plastids, in a non-human organism or a part thereof; preferably a microorganism, a plant cell, a plant or a part thereof, through transformation of the organelle, as compared to a corresponding non-transformed wild type non-human organism or a part thereof;
   (wherein in the respective line in the respective Table in column 7 glutamine is depicted) and
(b) growing the non-human organism under conditions which permit the production of glutamine, or a composition comprising glutamine in said non-human organism or in the culture medium surrounding said non-human organism.

A further embodiment of the present invention relates to a process for the production of proline, which comprises
(a1) increasing or generating the activity of a polypeptide comprising a polypeptide as depicted in the respective line in column 5 or 8 of Table II, application no. 2, or a homolog or fragment thereof, a consensus sequence or at least one polypeptide motif as depicted in column 8 of Table IV, application no. 2; or increasing or generating the activity of an expression product of one or more nucleic acid molecule(s) comprising a polynucleotide as depicted in the respective line in column 5 or 8 of Table I, application no. 2, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 2, or a homolog or a fragment thereof, a consensus sequence or at least one polypeptide motif as depicted in the respective line in column 8 of Table IV, application no. 2, which is joined to a transit peptide; or
increasing or generating the activity of an expression product of one or more nucleic acid molecule(s) comprising a polynucleotide as depicted in the respective line in column 5 or 8 of Table I, application no. 2, preferably the coding region thereof, or a homolog or a fragment thereof, which is joined to a nucleic acid sequence encoding an organelle localization sequence, preferably a plastid or a mitochondrion locatization sequence, especially a plastid localization sequence;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 2, or a homolog or a fragment thereof, a consensus sequence or at least one polypeptide motif as depicted in the respective line in column 8 of Table IV, application no. 2; or increasing or generating the activity of an expression product of one or more nucleic acid molecule(s) comprising a polynucleotide as depicted in the respective line in column 5 or 8 of Table I, application no. 2, preferably the coding region thereof, or a homolog or a fragment thereof;
in an organelle, preferably in plastids or imitochondra, especially in plastids, in a non-human organism or a part thereof; preferably a microorganism, a plant cell, a plant or a part thereof, through transformation of the organelle, as compared to a corresponding non-transformed wild type non-human organism or a part thereof;
   (wherein in the respective line in the respective Table in column 7 proline is depicted) and
(b) growing the non-human organism under conditions which permit the production of proline, or a composition comprising proline in said non-human organism or in the culture medium surrounding said non-human organism.

Accordingly, the present invention relates to a process for the production of glutamate, which comprises
(a) increasing or generating the activity of a 47266012-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a 47266012-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating the activity of a 49747384_SOYBEAN-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamate, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of arginine, which comprises
(a) increasing or generating the activity of a glucose-6-phosphate 1-dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating the activity of a glucose-6-phosphate 1-dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a amino acid acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating the activity of a At1g17440-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating the activity of a At1g19800-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating the activity of a cullin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating the activity of a cullin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a At1g29350-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of arginine, which comprises
(a) increasing or generating the activity of a eukaryotic translation initiation factor 5 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating the activity of a eukaryotic translation initiation factor 5 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a At1g47380-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a At1g67340-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism. Accordingly, the present invention relates to a process for the production of proline, which comprises
(a) increasing or generating the activity of a serine protease inhibitor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a CBL-interacting protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a CBL-interacting protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a CBL-interacting protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a delta-1-pyrroline 5-carboxylase synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a phosphatidylinositol 3- and 4-kinase family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a ankyrin repeat family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of arginine, which comprises
(a) increasing or generating the activity of a thioredoxin family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating the activity of a thioredoxin family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a thioredoxin family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a thioredoxin family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a harpin-induced family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating the activity of a harpin-induced family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a harpin-induced family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a DNA binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a DNA mismatch repair protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a CCAAT-binding transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a glucose-6-phosphate 1-dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a monthiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a monthiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamate, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a At4g32480-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating the activity of a At4g32480-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a At4g32480-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of arginine, which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating the activity of a gibberellin 20-oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a integral membrane transporter family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a integral membrane transporter family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating the activity of a At5g16650-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating the activity of a At5g16650-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a At5g16650-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating the activity of a translation initiation factor subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating the activity of a zinc finger protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating the activity of a zinc finger protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamate, which comprises
(a) increasing or generating the activity of a adenylylsulfate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a adenylylsulfate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating the activity of a malic enzyme in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a malic enzyme in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a glycosyl transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a sec-independent protein translocase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of arginine, which comprises
(a) increasing or generating the activity of a beta-hydroxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of arginine, which comprises
(a) increasing or generating the activity of a elongation factor Tu in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating the activity of a elongation factor Tu in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a elongation factor Tu in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating the activity of a ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a acyl-CoA synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating the activity of a acyl-CoA synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a acyl-CoA synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a acyl-CoA synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a 2-oxoglutarate dehydrogenase E1 subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of arginine, which comprises
(a) increasing or generating the activity of a enoyl-CoA hydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating the activity of a enoyl-CoA hydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a acyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating the activity of a lysyl-tRNA synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating the activity of a 30S ribosomal protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamate, which comprises
(a) increasing or generating the activity of a CTP synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a CTP synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating the activity of a glucose-1-phosphate cytidylyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a glucose-1-phosphate cytidylyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a glucose-1-phosphate cytidylyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a aminotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating the activity of a hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a glutamate-ammonia-ligase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating the activity of a glutamate-ammonia-ligase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a glutamate-ammonia-ligase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a fumarylacetoacetate hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a HesB/YadR/YfhF family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating the activity of a HesB/YadR/YfhF family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a HesB/YadR/YfhF family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a L-aspartate oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a Chaperone protein CIpB in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a purine nucleoside phosphorylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating the activity of a purine nucleoside phosphorylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a purine nucleoside phosphorylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a transcriptional regulator protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a AX653549-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a AX653549-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating the activity of a AY087308-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating the activity of a threonine synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating the activity of a threonine synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a threonine synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a L-ribulose-5-phosphate 4-epimerase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a dihydrolipoamide acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating the activity of a glucose dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating the activity of a glucose dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a glucose dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a serine protease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a acyl-CoA dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating the activity of a acyl-CoA dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating the activity of a beta-galactosidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a ATP-binding component of a transport system in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a b0456-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a membrane transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating the activity of a membrane transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a b0518-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating the activity of a isochorismate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating the activity of a isochorismate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a isochorismate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a isochorismate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a zinc transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating the activity of a asparaginase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating the activity of a Leucyl/phenylalanyl-tRNA-protein transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a Leucyl/phenylalanyl-tRNA-protein transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a major facilitator superfamily transporter protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating the activity of a major facilitator superfamily transporter protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a DNA helicase IV in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a methylglyoxal synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a phosphoanhydride phosphorylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a b1003-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a b1003-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a lipoprotein precursor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating the activity of a b1024-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a b1108-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a b1108-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a isocitrate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a b1137-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating the activity of a b1137-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating the activity of a b1137-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a b1163-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a sodium/proton antiporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a membrane protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a membrane protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a b1259-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating the activity of a b1259-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating the activity of a anthranilate synthase component II in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating the activity of a b1280-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating the activity of a glutamine synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating the activity of a aldehyde dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamate, which comprises
(a) increasing or generating the activity of a b1330-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a b1330-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating the activity of a lipoprotein precursor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a lipoprotein precursor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating the activity of a b1445-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a b1445-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating the activity of a b1522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a acid shock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating the activity of a electron transport complex protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating the activity of a protease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamate, which comprises
(a) increasing or generating the activity of a b1898-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating the activity of a transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a uridine/cytidine kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating the activity of a b2107-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a b2121-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating the activity of a ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a NADH dehydrogenase I chain I in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating the activity of a b2360-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating the activity of a b2399-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of arginine, which comprises
(a) increasing or generating the activity of a cysteine synthase A in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating the activity of a cysteine synthase A in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating the activity of a ethanolamine utilization protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a b2474-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a b2513-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a b2513-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating the activity of a 2,3-dihydroxy-2,3-dihydrophenylpropionatedehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating the activity of a 2,3-dihydroxy-2,3-dihydrophenylpropionatedehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a b2548-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating the activity of a b2613-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a b2613-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a b2673-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a b2673-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a b2812-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a b2812-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a b2812-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a b2846-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a b2909-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating the activity of a b2909-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a ribosephosphate isomerase, constitutive in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamate, which comprises
(a) increasing or generating the activity of a arginine exporter protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating the activity of a b2936-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a L-asparaginase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a murein transglycosylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a b2999-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating the activity of a glycoprotease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a b3121-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating the activity of a b3121-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a b3151-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a acetyl CoA carboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating the activity of a acetyl CoA carboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of arginine, which comprises
(a) increasing or generating the activity of a methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating the activity of a methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a b3346-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a b3410-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating the activity of a B3427-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating the activity of a b3509-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating the activity of a valine-pyruvate transaminase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a threonine dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a threonine dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a phosphopantetheine adenylyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a ATP synthase subunit beta in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating the activity of a enterobacterial common antigen polymerase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating the activity of a enterobacterial common antigen polymerase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating the activity of a DNA helicase II in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a b3814-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a b3817-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a glycerol dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating the activity of a b3989-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of arginine, which comprises
(a) increasing or generating the activity of a b4029-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating the activity of a b4029-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a b4029-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a cation/acetate symporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of arginine, which comprises
(a) increasing or generating the activity of a b4121-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating the activity of a b4121-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a lysyl-tRNA synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a aspartase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a gluconate transport system permease 3 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating the activity of a purine-nucleoside phosphorylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating the activity of a purine-nucleoside phosphorylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a thioredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating the activity of a GM02LC11114-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating the activity of a histone H2A in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating the activity of a histone H2A in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a GM02LC17485-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating the activity of a RNA binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a GM02LC46-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a GM02LC5744-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a s_pp015018333r-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating the activity of a s_pp015018333r-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a sll0064-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a N-acetyl-gamma-glutamyl-phosphate reductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamate, which comprises
(a) increasing or generating the activity of a heat shock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of arginine, which comprises
(a) increasing or generating the activity of a flavodoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating the activity of a phosphopantothenoylcysteinesynthetase/decarboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a sll0254-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a sll0254-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a polyphosphate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a sll0354-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a urease subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a NAD(P)H-quinone oxidoreductase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating the activity of a quinolinate synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a permease protein of phosphate ABC transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating the activity of a permease protein of phosphate ABC transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a oxireductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating the activity of a oxireductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a oxireductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating the activity of a malate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating the activity of a malate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a malate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a phosphoribosylaminoimidazole carboxylase catalytic subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a 3-deoxy-7-phosphoheptulonate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a glycogen synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a carbon dioxide concentrating mechanism protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a carbon dioxide concentrating mechanism protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a carbon dioxide concentrating mechanism protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a phosphoribosylformyl glycinamidine synthase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a coproporphyrinogen oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a glycogen (starch) synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a glycogen (starch) synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a fatty acid desaturase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating the activity of a CTP synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamate, which comprises
(a) increasing or generating the activity of a nitrate/nitrite transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamate, which comprises
(a) increasing or generating the activity of a CDP-diacylglycerol-glycerol-3-phosphate 3-phosphatidyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of arginine, which comprises
(a) increasing or generating the activity of a glutathione S-transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating the activity of a glutathione S-transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a glutathione S-transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a exopolyphosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating the activity of a 4-alpha-glucanotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a alanine dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a sll1761-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating the activity of a sll1761-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a coproporphyrinogen III oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a coproporphyrinogen III oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a coproporphyrinogen Ill oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a cation-transporting ATPase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a cation-transporting ATPase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating the activity of a glycosidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating the activity of a glycosidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a phosphoribosylglycinamide formyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a bifunctional purine biosynthesis protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating the activity of a slr0600-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a pyrroline carboxylate reductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a Glu/Leu/PheNal dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of arginine, which comprises
(a) increasing or generating the activity of a geranylgeranyl pyrophosphate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating the activity of a geranylgeranyl pyrophosphate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a geranylgeranyl pyrophosphate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a circadian clock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating the activity of a circadian clock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a amine oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a dihydrolipoamide dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of arginine, which comprises
(a) increasing or generating the activity of a gamma-glutamyltranspeptidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating the activity of a gamma-glutamyltranspeptidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of arginine, which comprises
(a) increasing or generating the activity of a arginine decarboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating the activity of a CDP-diglyceride synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamate, which comprises
(a) increasing or generating the activity of a carbohydrate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a iron(III) dicitrate-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a Photosystem I reaction center subunit XI in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a photosystem II protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating the activity of a photosystem II protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a glutamine amidotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a glycerol-3-phosphate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating the activity of a phosphoadenosine phosphosulfate reductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a riboflavin biosynthesis protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamate, which comprises
(a) increasing or generating the activity of a malonyl CoA-acyl carrier protein transacylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a malonyl CoA-acyl carrier protein transacylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a short-chain alcohol dehydrogenase family in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a Sec-independent protein translocase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a Sec-independent protein translocase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a cell division protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a branched-chain amino acid ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a amino acid ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a TTC0768-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a metal-dependent hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating the activity of a multiple antibiotic resistance protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamate, which comprises
(a) increasing or generating the activity of a TTC1386-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a homocitrate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a homocitrate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a oxidoreductase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a oxidoreductase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating the activity of a XM_{—}473199-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a XM_{—}473199-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a cell division control protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating the activity of a cell division control protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a YCL026C-A-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a ycr102c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a coproporphyrinogen Ill oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a branched-chain amino acid permease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a peroxisome assembly protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of arginine, which comprises
(a) increasing or generating the activity of a ydr338c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating the activity of a yer014w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamine, which comprises
(a) increasing or generating the activity of a yer106w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a molecular chaperone portein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a YFL019C-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a yfl054c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating the activity of a yfl054c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating the activity of a pre-mRNA-splicing factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating the activity of a ygl237c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a ygr068c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a squalene monooxygenase in the cytosol of a cell of a non-human organism or a part thereof, preferably a microorganism, a plant cell, a plant or a part thereof, as compared to a corresponding non-transformed wild type non-human organism or a part thereof; and (b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a ygr221c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a ygr221c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a yhl013c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a mitochondrial processing protease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating the activity of a H/ACA ribonucleoprotein complex subunit 3 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a yhr207c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of arginine, which comprises
(a) increasing or generating the activity of a 3-phosphoglycerate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating the activity of a allantoinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a allantoicase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating the activity of a DnaJ-like chaperone in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of arginine, which comprises
(a) increasing or generating the activity of a ornithine carbamoyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating the activity of a potassium transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a glycogenin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating the activity of a ATP-dependent RNA helicase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a phosphoribosyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of glutamate, which comprises
(a) increasing or generating the activity of a polygalacturonase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of proline, which comprises
(a) increasing or generating the activity of a aspartate aminotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamate, which comprises
(a) increasing or generating the activity of a ylr065c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a ylr178c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a aconitate hydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a adenylosuccinate lyase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of glutamine, which comprises
(a) increasing or generating the activity of a ynl142w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a sterol O-acyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamate, which comprises
(a) increasing or generating the activity of a glutaminyl-tRNA synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamate or a composition comprising glutamate in said non-human organism or in the culture medium surrounding said non-human organism.

Accordingly, the present invention relates to a process for the production of glutamine, which comprises
(a) increasing or generating the activity of a glutaminyl-tRNA synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a yor221c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of proline, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a isopentenyl diphosphate isomerase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of arginine, which comprises
(a) increasing or generating the activity of a Zm_{—}4842_BE510522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of glutamine, which comprises
(a) increasing or generating the activity of a Zm_{—}4842_BE510522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glutamine or a composition comprising glutamine in said non-human organism or in the culture medium surrounding said non-human organism.

Accordingly, the present invention relates to a process for the production of proline, which comprises
(a) increasing or generating the activity of a Zm_{—}4842_BE510522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a phosphopantothenoylcysteine decarboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine or a composition comprising arginine in said non-human organism or in the culture medium surrounding said non-human organism.

Accordingly, the present invention relates to a process for the production of proline, which comprises
(a) increasing or generating the activity of a ZM06LC11975-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of proline or a composition comprising proline in said non-human organism or in the culture medium surrounding said non-human organism.

Accordingly, the present invention relates to a process for the production of arginine, which comprises
(a) increasing or generating the activity of a polypeptide comprising a polypeptide as depicted in the respective line in column 5 or 8 of Table II, application no. 2, or a homolog or a fragment thereof, a consensus sequence or at least one polypeptide motif as depicted in the respective line in column 8 of Table IV, application no. 2; or increasing or generating the activity of an expression product of one or more nucleic acid molecule(s) comprising a polynucleotide as depicted in the respective line in column 5 or 8 of Table I, application no. 2, preferably the coding region thereof, or a homolog or a fragment thereof;
in the cytosol of a cell of a non-human organism or a part thereof; preferably a microorganism, a plant cell, a plant or a part thereof, as compared to a corresponding non-transformed wild type non-human organism or a part thereof;
(wherein in the respective line in the respective Table in column 7 arginine is depicted) and
(b) growing the non-human organism under conditions which permit the production of arginine, or a composition comprising arginine in said non-human organism or in the culture medium surrounding said non-human organism.

Accordingly, the present invention relates to a process for the production of glutamate, which comprises
(a) increasing or generating the activity of a polypeptide comprising a polypeptide as depicted in the respective line in column 5 or 8 of Table II, application no. 2, or a homolog or a fragment thereof, a consensus sequence or at least one polypeptide motif as depicted in the respective line in column 8 of Table IV, application no. 2; or increasing or generating the activity of an expression product of one or more nucleic acid molecule(s) comprising a polynucleotide as depicted in the respective line in column 5 or 8 of Table I, application no. 2, preferably the coding region thereof, or a homolog or a fragment thereof;
in the cytosol of a cell of a non-human organism or a part thereof; preferably a microorganism, a plant cell, a plant or a part thereof, as compared to a corresponding non-transformed wild type non-human organism or a part thereof;
(wherein in the respective line in the respective Table in column 7 glutamate is depicted) and
(b) growing the non-human organism under conditions which permit the production of glutamate, or a composition comprising glutamate in said non-human organism or in the culture medium surrounding said non-human organism.

Accordingly, the present invention relates to a process for the production of glutamine, which comprises
(a) increasing or generating the activity of a polypeptide comprising a polypeptide as depicted in the respective line in column 5 or 8 of Table II, application no. 2, or a homolog or a fragment thereof, a consensus sequence or at least one polypeptide motif as depicted in the respective line in column 8 of Table IV, application no. 2; or increasing or generating the activity of an expression product of one or more nucleic acid molecule(s) comprising a polynucleotide as depicted in the respective line in column 5 or 8 of Table I, application no. 2, preferably the coding region thereof, or a homolog or a fragment thereof;
in the cytosol of a cell of a non-human organism or a part thereof; preferably a microorganism, a plant cell, a plant or a part thereof, as compared to a corresponding non-transformed wild type non-human organism or a part thereof;
(wherein in the respective line in the respective Table in column 7 glutamine is depicted) and
(b) growing the non-human organism under conditions which permit the production of glutamine, or a composition comprising glutamine in said non-human organism or in the culture medium surrounding said non-human organism.

Accordingly, the present invention relates to a process for the production of proline, which comprises
(a) increasing or generating the activity of a polypeptide comprising a polypeptide as depicted in the respective line in column 5 or 8 of Table II, application no. 2, or a homolog or a fragment thereof, a consensus sequence or at least one polypeptide motif as depicted in the respective line in column 8 of Table IV, application no. 2; or
increasing or generating the activity of an expression product of one or more nucleic acid molecule(s) comprising a polynucleotide as depicted in the respective line in column 5 or 8 of Table I, application no. 2, preferably the coding region thereof, or a homolog or a fragment thereof;
in the cytosol of a cell of a non-human organism or a part thereof; preferably a microorganism, a plant cell, a plant or a part thereof, as compared to a corresponding non-transformed wild type non-human organism or a part thereof;
(wherein in the respective line in the respective Table in column 7 proline is depicted) and
(b) growing the non-human organism under conditions which permit the production of proline, or a composition comprising proline in said non-human organism or in the culture medium surrounding said non-human organism.

In a further embodiment the activity of the polypeptide comprising a polypeptide as depicted in the respective line in column 5 or 8 of Table II, application no. 2, or a homolog or a fragment thereof, a consensus sequence or at least one polypeptide motif as depicted in the respective line in column 8 of Table IV, application no. 2, is increased or generated non-targeted in the above-mentioned process in a microorganism or plant or a part thereof.

In a further embodiment said polypeptide has the activity of the respective polypeptide represented by a protein comprising a polypeptide as depitcted in the respective line in column 5 of Table II, application no. 2.

In a further embodiment the activity of the expression product of one or more nucleic acid molecule(s) comprising a polynucleotide as depicted in the respective line in column 5 or 8 of Table I, application no. 2, preferably the coding region thereof, or a homolog or a fragment thereof, is increased or generated non-targeted in the above-mentioned process in a microorganism or plant or a part thereof.

In a further embodiment the activity of the polypeptide comprising a polypeptide as depicted in the respective line in column 5 or 8 of Table II, application no. 2, or a homolog or a fragment thereof, a consensus sequence or at least one polypeptide motif as depicted in the respective line in column 8 of Table IV, application no. 2, is increased or generated in the above-mentioned process in an organelle preferably in plastids or mitochondria, especially in plastids, of a microorganism or plant.

In a further embodiment said polypeptide has the activity of the respective polypeptide represented by a protein comprising a polypeptide as depitcted in the respective line in column 5 of Table II, application no. 2.

In a further embodiment the activity of the expression product of one or more nucleic acid molecule(s) comprising a polynucleotide as depicted in the respective line in column 5 or 8 of Table I, application no. 2, preferably the coding region thereof, or a homolog or a fragment thereof, is increased or generated in the above-mentioned process in an organelle, preferably in plasids or mitochondria, especially in plastids, of a microorganism or plant.

In a further embodiment the activity of the polypeptide comprising a polypeptide as depicted in the respective line in column 5 or 8 of Table II, application no. 2, or a homolog or a fragment thereof, a consensus sequence or at least one polypeptide motif as depicted in the respective line in column 8 of Table IV, application no. 2, is increased or generated in the above-mentioned process in the cyctosol of a cell, of a microorganism or plant.

In a further embodiment said polypeptide has the activity of the respective polypeptide represented by a protein comprising a polypeptide as depitcted in the respective line in column 5 of Table II, application no. 2.

In a further embodiment the activity of the expression product of one or more nucleic acid molecule(s) comprising a polynucleotide as depicted in the respective line in column 5 or 8 of Table I, application no. 2, preferably the coding region thereof, or a homolog or a fragment thereof, is increased or generated in the above-mentioned process in the cytosol of a cell, of a microorganism or plant.

[0039.1.1.2] to [0066.1.1.2] for the disclosure of these paragraphs see [0039.1.1.1] to [0066.1.1.1] above.

As mentioned above the nucleic acid sequences coding for the proteins as shown in Table II, application no. 2, column 5 or 8, or homologs or fragments thereof, are joined to a nucleic acid sequence encoding a plastidic transit peptide, e.g, if for the nucleic acid molecule in Table I, column 6 the term “plastidic”is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in Table II, application no. 2, column 5 or 8, or homologs or fragments thereof, e.g, if for the nucleic acid molecule in Table I, column 6 the term “plastidic” is indicated.

[0068.1.1.2] to [0072.1.1.2] for the disclosure of these paragraphs see [0068.1.1.1] to [0072.1.1.1] above.

As mentioned above the nucleic acid sequences coding for the proteins as shown in the respective line in Table II, application no. 2, column 5 or 8, or homologs or fragments thereof, are joined to a nucleic acid sequence encoding a mitochondrial transit peptide, e.g., if for the respective nucleic acid molecule in Table I, column 6 the term “mitochondrial” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in the respective line in Table II, application no. 2, column 5 or 8, or homologs or fragments thereof, e.g, if for the nucleic acid molecule in the respective line in Table I, column 6 the term “mitochondrial” is indicated. (For the avoidance of doubt, the term “mitochondric” and “mitochondrial” are interchangeable.)

[0074.1.1.2] to [0075.1.1.2] for the disclosure of these paragraphs see [0074.1.1.1] to [0075.1.1.1] above.

Nucleic acid sequences coding for the transit peptides may be chemically synthesized either in part or wholly according to structure of transit peptide sequences disclosed in the prior art. Said natural or chemically synthesized sequences can be directly linked to the sequences encoding the mature protein or via a linker nucleic acid sequence, which may be typically less than 500 base pairs, preferably less than 450, 400, 350, 300, 250 or 200 base pairs, more preferably less than 150, 100, 90, 80, 70, 60, 50, 40 or 30 base pairs and most preferably less than 25, 20, 15, 12, 9, 6 or 3 base pairs in length and are in frame to the coding sequence. Furthermore favorable nucleic acid sequences encoding transit peptides may comprise sequences derived from more than one biological and/or chemical source and may include a nucleic acid sequence derived from the amino-terminal region of the mature protein, which in its native state is linked to the transit peptide. In a preferred embodiment of the invention said amino-terminal region of the mature protein is typically less than 150 amino acids, preferably less than 140, 130, 120, 110, 100 or 90 amino acids, more preferably less than 80, 70, 60, 50, 40, 35, 30, 25 or 20 amino acids and most preferably less than 19, 18, 17, 16, 15, 14, 13, 12, 11 or 10 amino acids in length. But even shorter or longer stretches are also possible. In addition target sequences, which facilitate the transport of proteins to other cell compartments such as the vacuole, endoplasmic reticulum, golgi complex, glyoxysomes, peroxisomes, mitochondria (in case of a plastidal transit peptide) or plastids, especially chloroplasts (in case of a mitochondric transit peptide) may be also part of the inventive nucleic acid sequence. The proteins translated from said inventive nucleic acid sequences are a kind of fusion proteins that means the nucleic acid sequences encoding the plastidal transit peptide for example the ones shown in Table a, preferably the last one of the Table a are joint to the respective nucleic acid sequences shown in Table I, application no. 2, columns 5 or 8, especially the coding region thereof, or homologs or fragments thereof, in case of nucleic acid sequences being expressed in plastids, and that means the nucleic acid sequences encoding the mitochondric transit peptide for example the ones shown in Table b, preferably the last one of the Table b are joint to the respective nucleic acid sequences shown in Table I, application no. 2, columns 5 or 8, especially the coding region thereof, or homologs or fragments thereof, in case of nucleic acid sequences being expressed in mitochondria. The person skilled in the art is able to join said sequences in a functional manner. Advantageously the transit peptide part is cleaved off from the protein part shown in Table II, application no. 2, columns 5 or 8, or a homolog or fragment thereof, during or after the transport into the organelles, preferably into the plastids or mitochondria, respectively.

All products of the cleavage of the preferred transit peptide shown in the last line of Table a have preferably the N-terminal amino acid sequences QIA CSS or QIA EFQLTT in front of the start methionine of the respective protein metioned in Table II, application no. 2, column 5 or 8. or homologs or fragments thereof. Other short amino acid sequences of an range of 1 to 20 amino acids preferable 2 to 15 amino acids, more preferable 3 to 10 amino acids most preferably 4 to 8 amino acids are also possible in front of the start methionine of the protein metioned in Table II, application no. 2, columns 5 or 8, or homologs or fragments thereof. In case of the amino acid sequence QIA CSS the three amino acids in front of the start methionine are stemming from the LIC (=ligatation independent cloning) cassette. Said short amino acid sequence is preferred in the case of the expression of E. coli genes. In case of the amino acid sequence QIA EFQLTT the six amino acids in front of the start methionine are stemming from the LIC cassette. Said short amino acid sequence is preferred in the case of the expression of S. cerevisiae genes. The skilled worker knowns that other short sequences are also useful in the expression of the respective nucleic acid molecule comprising a polynucleotide as depicted in Table I, application no. 2, column 5 or 8, preferably the coding region thereof, or a homolog or a fragment thereof. Furthermore the skilled worker is aware of the fact that there is not a need for such short sequences in the expression of the genes.

[0077.1.1.2] to [0078.1.1.2] for the disclosure of these paragraphs see [0077.1.1.1] to [0078.1.1.1] above.

Alternatively to the targeting of the respective sequences shown in Table II, application no. 2, columns 5 or 8, or homologs or fragments thereof, preferably of sequences in general encoded in the nucleus with the aid of the targeting sequences mentioned for example in Table a alone, Table b alone, or in combination with other targeting sequences preferably into plastids or mitochondria, respectively, the nucleic acids of the invention can directly be introduced into the plastidal or mitochondrial genome. Therefore in a preferred embodiment the respective nucleic acid sequences shown in the respective line in Table I, application no. 2, columns 5 or 8, preferably the coding region thereof, or homologs or fragments thereof, are directly introduced and expressed in plastids e.g, if for the nucleic acid molecule in the respective line in Table I, column 6, the term “plastidic” is indicated, or are directly introduced and expressed in mitochondria e.g., if for the nucleic acid molecule in the respective line in Table I, column 6 the term “mitochondrial” is indicated.

The term “introduced” in the context of this specification shall mean the insertion of a nucleic acid sequence into the organism by means of a “transfection”, “transduction” or preferably by “transformation”.

A plastid, such as a chloroplast, or a mitochondrion, has been “transformed” by an exogenous (preferably foreign) nucleic acid sequence if a nucleic acid sequence has been introduced into the plastid or mitochondrion that means that this sequence has crossed the membrane or the membranes of the plastid or of the mitochondrion. The exogenous, preferably foreign, DNA may be integrated (covalently linked) into plastid or mitochondrial DNA making up the genome of the plastid or mitochondrion, or it may remain unintegrated (e.g., by including a chloroplast origin of replication). “Stably” integrated DNA sequences are those, which are inherited through plastid or mitochondrion replication, thereby transferring new plastids or mitochondria, with the features of the integrated DNA sequence to the progeny.

[0080.1.1.2] to [0083.1.1.2] for the disclosure of these paragraphs see [0080.1.1.1] to [0083.1.1.1] above.

For the process of the present invention it may be of great advantage that by transforming the plastids the intraspecies specific transgene flow is blocked, because a lot of species such as corn, cotton and rice have a strict maternal inheritance of plastids. By placing the respective genes specified in the respective line in Table I, application no. 2, columns 5 or 8, preferably the coding region, or homologs or fragments thereof, e.g. if for the nucleic acid molecule in the respective line in column 6 of Table I the term “plastidic” is indicated, in the plastids of plants, these genes will not be present in the pollen of said plants.

A further preferred embodiment of the invention relates to the use of so called “chloroplast localization sequences”, in which a first RNA sequence or molecule is capable of transporting or “chaperoning” a second RNA sequence, such as a RNA sequence transcribed from the sequences depicted in the respective line in Table I, application no. 2, columns 5 or 8, preferably the coding region theeof, or a homolog or a fragment thereof, or a sequence encoding a protein, as depicted in the respective line in Table II, application no. 2, columns 5 and 8, or a homolog or a fragment thereof, from an external environment inside a cell or outside a plastid into a chloroplast. In one embodiment the chloroplast localization signal is substantially similar or complementary to a complete or intact viroid sequence. The chloroplast localization signal may be encoded by a DNA sequence, which is transcribed into the chloroplast localization RNA. The term “viroid” refers to a naturally occurring single stranded RNA molecule (Flores, C. R. Acad. Sci. III. 324 (10), 943 (2001)). Viroids usually contain about 200-500 nucleotides and generally exist as circular molecules. Examples of viroids that contain chloroplast localization signals include but are not limeted to ASBVd, PLMVd, CChMVd and ELVd. The viroid sequence or a functional part of it can be fused to the sequences depicted in the respective line in Table I, application no. 2, columns 5 or 8, preferably the coding region thereof, or a homolog or a fragment thereof, or a sequence encoding a protein, as depicted in the respective line in Table II, application no. 2, columns 5 or 8, or a homolog or a fragment thereof, in such a manner that the viroid sequence transports a sequence transcribed from a sequence as depicted in th erespective line in Table I, application no. 2 columns 5 or 8, preferably the coding region thereof, or a homolog or a fragment thereof, or a sequence encoding a protein as depicted in th erespective line in Table II, application no. 2 columns 5 or 8, or a homolog or a fragment thereof, into the chloroplasts, e.g. if for the nucleic acid molecule in column 6 of Table I the term “plastidic” is indicated. A preferred embodiment uses a modified ASBVd (Navarro et al., Virology. 268 (1), 218 (2000)).

In a further specific embodiment the protein to be expressed in the plastids such as the proteins depicted in the respective line in Table II, application no. 2, columns 5 or 8, or a homolog or a fragment thereof, e.g. if for the nucleic acid molecule in the respective line in column 6 of Table I the term “plastidic” is indicated, are encoded by different nucleic acids. Such a method is disclosed in WO 2004/040973, which shall be incorporated by reference. WO 2004/040973 teaches a method, which relates to the translocation of an RNA corresponding to a gene or gene fragment into the chloroplast by means of a chloroplast localization sequence. The genes, which should be expressed in the plant or plant cells, are split into nucleic acid fragments, which are introduced into different compartments in the plant e.g. the nucleus, the plastids and/or mitochondria. Additionally plant cells are described in which the chloroplast contains a ribozyme fused at one end to an RNA encoding a fragment of a protein used in the inventive process such that the ribozyme can trans-splice the translocated fusion RNA to the RNA encoding the gene fragment to form and as the case may be reunite the nucleic acid fragments to an intact mRNA encoding a functional protein for example as disclosed in the respective line in Table II, application no. 2, columns 5 or 8, or a homolog or a fragment thereof.

In a preferred embodiment of the invention the nucleic acid sequences as shown in the respective line in Table I, application no. 2, columns 5 or 8, preferably the coding region thereof, or homologs or fragments thereof, e.g. if for the nucleic acid molecule in the respective line in column 6 of Table I the term “plastidic” is indicated, used in the inventive process are transformed into plastids, which are metabolical active. Those plastids should preferably maintain at a high copy number in the plant or plant tissue of interest, most preferably the chloroplasts found in green plant tissues, such as leaves or cotyledons or in seeds.

For a good expression in the plastids the nucleic acid sequences as shown in th erespective line in Table I, application no. 2, columns 5 or 8, preferably the coding region thereof, or homologs or fargments thereof, e.g. if for the nucleic acid molecule in the respective line in column 6 of Table I the term “plastidic” is indicated, are introduced into an expression cassette using preferably a promoter and terminater, which are active in plastids preferably a chloroplast promoter. Examples of such promoters include the psbA promoter from the gene from spinach or pea, the rbcL promoter, and the atpB promoter from corn.

[0089.1.1.2] to [0092.1.1.2] for the disclosure of these paragraphs see [0089.1.1.1] to [0092.1.1.1] above.

Advantageously the process for the production of the fine chemical arginine, glutamate, glutamine, and/or proline leads to an enhanced production of the respective fine chemical. The terms “enhanced” or “increase” mean at least a 10%, 20%, 30%, 40% or 50%, preferably at least 60%, 70%, 80%, 90% or 100%, more preferably 150%, 200%, 300%, 400% or 500% higher production of the respective fine chemical arginine, glutamate, glutamine, and/or proline in comparison to the wild-type as defined above, e.g. that means in comparison to a non-human organism without the aforementioned modification of the activity of a protein as shown in the respective line in Table II, application no. 2, column 5 or 8, or a fragment or a homolog thereof. The modification of the activity of a protein as shown in the respective line in Table II, application no. 2, column 5 or 8, or a homolog or a fragment thereof, or their combination can be achieved by joining the protein to a respective transit peptide, e.g. if for the respective encoding nucleic acid molecule in column 6 of Table I the term “plastidic” or “mitochondrial” is indicated.

Surprisingly it was found, that the transgenic expression of a protein as shown in the respective line in Table II, application no. 2, column 5, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, conferred a production of or an increase in arginine, glutamate, glutamine, and/or proline,respectively, to the transgenic non-human organism as compared to a corresponding non-transformed wild type.

In one embodiment the transgenic expression of a protein as shown in the respective line in Table II, application no. 2, column 5 or 8, or a homolog or a fragment thereof, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, confers a production of or an increase in arginine, glutamate, glutamine, and/or proline, respectively, to the transgenic non-human organism as compared to a corresponding non-transformed wild type.

Accordingly, in one embodiment, an increase of glutamate in a non-human organism, as compared to a corresponding non-transformed wild type non-human organism, is conferred in the process of the invention, if the activity of a polypeptide showing the activity of a 47266012-protein, or if the activity of the polypeptide 47266012_SOYBEAN, preferably represented by SEQ ID NO. 15188, or a homolog or fragment thereof, or if the activity of a polypeptide encoded by a nucleic acid molecule comprising the nucleic acid SEQ ID NO. 15187, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 15187 or polypeptide SEQ ID NO. 15188, respectively, or a homolog or a fragment thereof, is increased or generated, or if the activity 47266012-protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 42 to 96 -percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a 47266012-protein, or if the activity of the polypeptide 47266012_SOYBEAN, preferably represented by SEQ ID NO. 15188, or a homolog or fragment thereof, or if the activity of a polypeptide encoded by a nucleic acid molecule comprising the nucleic acid SEQ ID NO. 15187, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 15187 or polypeptide SEQ ID NO. 15188, respectively, or a homolog or a fragment thereof, is increased or generated, or if the activity 47266012-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 72 to 234-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a 49747384_SOYBEAN-protein, or if the activity of the polypeptide 49747384_SOYBEAN, preferably represented by SEQ ID NO. 70, or a homolog or fragment thereof, or if the activity of a polypeptide encoded by a nucleic acid molecule comprising the nucleic acid SEQ ID NO. 69, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 69 or polypeptide SEQ ID NO. 70, respectively, or a homolog or a fragment thereof, is increased or generated, or if the activity 49747384_SOYBEAN-protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 37 to 122-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a pyruvate kinase, or if the activity of the polypeptide 51340801_CANOLA, preferably represented by SEQ ID NO. 15533, or a homolog or fragment thereof, or if the activity of a polypeptide encoded by a nucleic acid molecule comprising the nucleic acid SEQ ID NO. 15532, preferably the coding region thereof, or a homolog or fragment thereof, e.g. derived from Brassica napus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 15532 or polypeptide SEQ ID NO. 15533, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line discloses in column 7 glutamate. For example, an increase of the glutamate of at least 1 percent, particularly in a range of 35 to 135-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a pyruvate kinase, or if the activity of the polypeptide 51340801_CANOLA, preferably represented by SEQ ID NO. 15533, or a homolog or fragment thereof, or if the activity of a polypeptide encoded by a nucleic acid molecule comprising the nucleic acid SEQ ID NO. 15532, preferably the coding region thereof, or a homolog or fragment thereof, e.g. derived from Brassica napus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 15532 or polypeptide SEQ ID NO. 15533, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line discloses in column 7 proline. For example, an increase of the proline of at least 1 percent, particularly in a range of 38 to 189-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a acetyltransferase, or if the activity of the polypeptide 59547452_SOYBEAN, preferably represented by SEQ ID NO. 16156, or a homolog or fragment thereof, or if the activity of a polypeptide encoded by a nucleic acid molecule comprising the nucleic acid SEQ ID NO. 16155, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 16155 or polypeptide SEQ ID NO. 16156, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 65 to 457-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a pyruvate kinase, or if the activity of the polypeptide 59554615_SOYBEAN, preferably represented by SEQ ID NO. 16264, or a homolog or fragment thereof, or if the activity of a polypeptide encoded by a nucleic acid molecule comprising the nucleic acid SEQ ID NO. 16263, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 16263 or polypeptide SEQ ID NO. 16264, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line discloses in column 7 glutamate. For example, an increase of the glutamate of at least 1 percent, particularly in a range of 44 to 80-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a glucose-6-phosphate 1-dehydrogenase, or if the activity of the polypeptide 59582753_SOYBEAN, preferably represented by SEQ ID NO. 16884, or a homolog or fragment thereof, or if the activity of a polypeptide encoded by a nucleic acid molecule comprising the nucleic acid SEQ ID NO. 16883, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 16883 or polypeptide SEQ ID NO. 16884, respectively, or a homolog or a fragment thereof, is increased or generated, or if the activity glucose-6-phosphate 1-dehydrogenase is increased or generated in a non-human organism, like a microorganism or a plant cell, plant or part thereof, especially with plastidic localization, whereby the respective line discloses in column 7 arginine. For example, an increase of the arginine of at least 1 percent, particularly in a range of 66 to 241-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a glucose-6-phosphate 1-dehydrogenase, or if the activity of the polypeptide 59582753_SOYBEAN, preferably represented by SEQ ID NO. 16884, or a homolog or fragment thereof, or if the activity of a polypeptide encoded by a nucleic acid molecule comprising the nucleic acid SEQ ID NO. 16883, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 16883 or polypeptide SEQ ID NO. 16884, respectively, or a homolog or a fragment thereof, is increased or generated, or if the activity glucose-6-phosphate 1-dehydrogenase is increased or generated in a non-human organism, like a microorganism or a plant cell, plant or part thereof, especially with plastidic localization, whereby the respective line discloses in column 7 proline. For example, an increase of the proline of at least 1 percent, particularly in a range of 34 to 204-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a amino acid acetyltransferase, or if the activity of the polypeptide AAC43185, preferably represented by SEQ ID NO. 17357, or a homolog or fragment thereof, or if the activity of a polypeptide encoded by a nucleic acid molecule comprising the nucleic acid SEQ ID NO. 17356, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 17356 or polypeptide SEQ ID NO. 17357, respectively, or a homolog or a fragment thereof, is increased or generated, or if the activity amino acid acetyltransferase is increased or generated in a non-human organism, like a microorganism or a plant cell, plant or part thereof, especially with plastidic localization, whereby the respective line discloses in column 7 arginine. For example, an increase of the arginine of at least 1 percent, particularly in a range of 586 to 3134-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a protein phosphatase, or if the activity of the polypeptide At1g07430, preferably represented by SEQ ID NO. 17452, or a homolog or fragment thereof, or if the activity of a polypeptide encoded by a nucleic acid molecule comprising the nucleic acid SEQ ID NO. 17451, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 17451 or polypeptide SEQ ID NO. 17452, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 39 to 67-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a protein phosphatase, or if the activity of the polypeptide At1g07430, preferably represented by SEQ ID NO. 17452, or a homolog or fragment thereof, or if the activity of a polypeptide encoded by a nucleic acid molecule comprising the nucleic acid SEQ ID NO. 17451, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 17451 or polypeptide SEQ ID NO. 17452, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 27 to 35-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a At1g17440-protein, or if the activity of the polypeptide At1g17440, preferably represented by SEQ ID NO. 17602, or a homolog or fragment thereof, or if the activity of a polypeptide encoded by a nucleic acid molecule comprising the nucleic acid SEQ ID NO. 17601, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 17601 or polypeptide SEQ ID NO. 17602, respectively, or a homolog or a fragment thereof, is increased or generated, or if the activity At1g17440-protein is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 54 to 181-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a At1g19800-protein, or if the activity of the polypeptide At1g19800, preferably represented by SEQ ID NO. 17638, or a homolog or fragment thereof, or if the activity of a polypeptide encoded by a nucleic acid molecule comprising the nucleic acid SEQ ID NO. 17637, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 17637 or polypeptide SEQ ID NO. 17638, respectively, or a homolog or a fragment thereof, is increased or generated, or if the activity At1g19800-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 53 to 175-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a cullin, or if the activity of the polypeptide At1g26830, preferably represented by SEQ ID NO. 17702, or a homolog or fragment thereof, or if the activity of a polypeptide encoded by a nucleic acid molecule comprising the nucleic acid SEQ ID NO. 17701, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 17701 or polypeptide SEQ ID NO. 17702, respectively, or a homolog or a fragment thereof, is increased or generated, or if the activity cullin is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 64 to 354-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a cullin, or if the activity of the polypeptide At1g26830, preferably represented by SEQ ID NO. 17702, or a homolog or fragment thereof, or if the activity of a polypeptide encoded by a nucleic acid molecule comprising the nucleic acid SEQ ID NO. 17701, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 17701 or polypeptide SEQ ID NO. 17702, respectively, or a homolog or a fragment thereof, is increased or generated, or if the activity cullin is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 65 to 193-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a At1g29350-protein, or if the activity of the polypeptide At1g29350, preferably represented by SEQ ID NO. 17902, or a homolog or fragment thereof, or if the activity of a polypeptide encoded by a nucleic acid molecule comprising the nucleic acid SEQ ID NO. 17901, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 17901 or polypeptide SEQ ID NO. 17902, respectively, or a homolog or a fragment thereof, is increased or generated, or if the activity At1g29350-protein is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 51 to 249-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a eukaryotic translation initiation factor 5, or if the activity of the polypeptide At1g36730, preferably represented by SEQ ID NO. 17969, or a homolog or fragment thereof, or if the activity of a polypeptide encoded by a nucleic acid molecule comprising the nucleic acid SEQ ID NO. 17968, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 17968 or polypeptide SEQ ID NO. 17969, respectively, or a homolog or a fragment thereof, is increased or generated, or if the activity eukaryotic translation initiation factor 5 is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 56 to 193-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a eukaryotic translation initiation factor 5, or if the activity of the polypeptide At1g36730, preferably represented by SEQ ID NO. 17969, or a homolog or fragment thereof, or if the activity of a polypeptide encoded by a nucleic acid molecule comprising the nucleic acid SEQ ID NO. 17968, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 17968 or polypeptide SEQ ID NO. 17969, respectively, or a homolog or a fragment thereof, is increased or generated, or if the activity eukaryotic translation initiation factor 5 is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 48 to 136-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a transcriptional regulator, or if the activity of the polypeptide At1g43850, preferably represented by SEQ ID NO. 18071, or a homolog or fragment thereof, or if the activity of a polypeptide encoded by a nucleic acid molecule comprising the nucleic acid SEQ ID NO. 18070, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 18070 or polypeptide SEQ ID NO. 18071, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 49 to 294-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a transcriptional regulator, or if the activity of the polypeptide At1g43850, preferably represented by SEQ ID NO. 18071, or a homolog or fragment thereof, or if the activity of a polypeptide encoded by a nucleic acid molecule comprising the nucleic acid SEQ ID NO. 18070, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 18070 or polypeptide SEQ ID NO. 18071, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 54 to 158-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a transcriptional regulator, or if the activity of the polypeptide At1g43850, preferably represented by SEQ ID NO. 18071, or a homolog or fragment thereof, or if the activity of a polypeptide encoded by a nucleic acid molecule comprising the nucleic acid SEQ ID NO. 18070, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 18070 or polypeptide SEQ ID NO. 18071, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 42 to 488-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a transcriptional regulator, or if the activity of the polypeptide At1g43850, preferably represented by SEQ ID NO. 18071, or a homolog or fragment thereof, or if the activity of a polypeptide encoded by a nucleic acid molecule comprising the nucleic acid SEQ ID NO. 18070, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 18070 or polypeptide SEQ ID NO. 18071, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 38 to 520-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a At1g47380-protein, or if the activity of the polypeptide At1g47380, preferably represented by SEQ ID NO. 18123, or a homolog or fragment thereof, or if the activity of a polypeptide encoded by a nucleic acid molecule comprising the nucleic acid SEQ ID NO. 18122, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 18122 or polypeptide SEQ ID NO. 18123, respectively, or a homolog or a fragment thereof, is increased or generated, or if the activity At1g47380-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 39 to 237-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a protein kinase, or if the activity of the polypeptide At1g48260, preferably represented by SEQ ID NO. 18236, or a homolog or fragment thereof, or if the activity of a polypeptide encoded by a nucleic acid molecule comprising the nucleic acid SEQ ID NO. 18235, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 18235 or polypeptide SEQ ID NO. 18236, respectively, or a homolog or a fragment thereof, is increased or generated, or if the activity protein kinase is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 70 to 338-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a protein kinase, or if the activity of the polypeptide At1g48260, preferably represented by SEQ ID NO. 18236, or a homolog or fragment thereof, or if the activity of a polypeptide encoded by a nucleic acid molecule comprising the nucleic acid SEQ ID NO. 18235, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 18235 or polypeptide SEQ ID NO. 18236, respectively, or a homolog or a fragment thereof, is increased or generated, or if the activity protein kinase is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 53 to 362-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a protein kinase, or if the activity of the polypeptide At1g48260, preferably represented by SEQ ID NO. 18236, or a homolog or fragment thereof, or if the activity of a polypeptide encoded by a nucleic acid molecule comprising the nucleic acid SEQ ID NO. 18235, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 18235 or polypeptide SEQ ID NO. 18236, respectively, or a homolog or a fragment thereof, is increased or generated, or if the activity protein kinase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 34 to 108-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a calciumdependent protein kinase, or if the activity of the polypeptide At1g61950, preferably represented by SEQ ID NO. 18870, or a homolog or fragment thereof, or if the activity of a polypeptide encoded by a nucleic acid molecule comprising the nucleic acid SEQ ID NO. 18869, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 18869 or polypeptide SEQ ID NO. 18870, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 89 to 385-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a calciumdependent protein kinase, or if the activity of the polypeptide At1g61950, preferably represented by SEQ ID NO. 18870, or a homolog or fragment thereof, or if the activity of a polypeptide encoded by a nucleic acid molecule comprising the nucleic acid SEQ ID NO. 18869, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 18869 or polypeptide SEQ ID NO. 18870, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 54 to 319-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a At1g67340-protein, or if the activity of the polypeptide At1g67340, preferably represented by SEQ ID NO. 19365, or a homolog or fragment thereof, or if the activity of a polypeptide encoded by a nucleic acid molecule comprising the nucleic acid SEQ ID NO. 19364, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 19364 or polypeptide SEQ ID NO. 19365, respectively, or a homolog or a fragment thereof, is increased or generated, or if the activity At1g67340-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 36 to 86-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a transcription factor, or if the activity of the polypeptide At1g68320, preferably represented by SEQ ID NO. 1062, or a homolog or fragment thereof, or if the activity of a polypeptide encoded by a nucleic acid molecule comprising the nucleic acid SEQ ID NO. 1061, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 1061 or polypeptide SEQ ID NO. 1062, respectively, or a homolog or a fragment thereof, is increased or generated, or if the activity transcription factor is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 57 to 183-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a protein phosphatase, or if the activity of the polypeptide At1g72770, preferably represented by SEQ ID NO. 19420, or a homolog or fragment thereof, or if the activity of a polypeptide encoded by a nucleic acid molecule comprising the nucleic acid SEQ ID NO. 19419, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 19419 or polypeptide SEQ ID NO. 19420, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 44 to 290-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a transcription factor, or if the activity of the polypeptide At2g17560, preferably represented by SEQ ID NO. 19503, or a homolog or fragment thereof, or if the activity of a polypeptide encoded by a nucleic acid molecule comprising the nucleic acid SEQ ID NO. 19502, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 19502 or polypeptide SEQ ID NO. 19503, respectively, or a homolog or a fragment thereof, is increased or generated, or if the activity transcription factor is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 39 to 181-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a protein phosphatase, or if the activity of the polypeptide At2g25070, preferably represented by SEQ ID NO. 1299, or a homolog or fragment thereof, or if the activity of a polypeptide encoded by a nucleic acid molecule comprising the nucleic acid SEQ ID NO. 1298, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 1298 or polypeptide SEQ ID NO. 1299, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 33 to 77-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a protein phosphatase, or if the activity of the polypeptide At2g25070, preferably represented by SEQ ID NO. 1299, or a homolog or fragment thereof, or if the activity of a polypeptide encoded by a nucleic acid molecule comprising the nucleic acid SEQ ID NO. 1298, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 1298 or polypeptide SEQ ID NO. 1299, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 60 to 213-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a serine protease inhibitor, or if the activity of the polypeptide At2g26390, preferably represented by SEQ ID NO. 19672, or a homolog or fragment thereof, or if the activity of a polypeptide encoded by a nucleic acid molecule comprising the nucleic acid SEQ ID NO. 19671, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 19671 or polypeptide SEQ ID NO. 19672, respectively, or a homolog or a fragment thereof, is increased or generated, or if the activity serine protease inhibitor is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 38 to 120-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a protein phosphatase, or if the activity of the polypeptide At2g28890, preferably represented by SEQ ID NO. 19875, or a homolog or fragment thereof, or if the activity of a polypeptide encoded by a nucleic acid molecule comprising the nucleic acid SEQ ID NO. 19874, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 19874 or polypeptide SEQ ID NO. 19875, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 36 to 716-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a CBL-interacting protein kinase, or if the activity of the polypeptide At2g30360, preferably represented by SEQ ID NO. 19920, or a homolog or fragment thereof, or if the activity of a polypeptide encoded by a nucleic acid molecule comprising the nucleic acid SEQ ID NO. 19919, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 19919 or polypeptide SEQ ID NO. 19920, respectively, or a homolog or a fragment thereof, is increased or generated, or if the activity CBL-interacting protein kinase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 46 to 170-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a CBL-interacting protein kinase, or if the activity of the polypeptide At2g30360, preferably represented by SEQ ID NO. 19920, or a homolog or fragment thereof, or if the activity of a polypeptide encoded by a nucleic acid molecule comprising the nucleic acid SEQ ID NO. 19919, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 19919 or polypeptide SEQ ID NO. 19920, respectively, or a homolog or a fragment thereof, is increased or generated, or if the activity CBL-interacting protein kinase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 43 to 132-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a glutaredoxin, or if the activity of the polypeptide At2g30540, preferably represented by SEQ ID NO. 20347, or a homolog or fragment thereof, or if the activity of a polypeptide encoded by a nucleic acid molecule comprising the nucleic acid SEQ ID NO. 20346, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 20346 or polypeptide SEQ ID NO. 20347, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 62 to 285-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a glutaredoxin, or if the activity of the polypeptide At2g30540, preferably represented by SEQ ID NO. 20347, or a homolog or fragment thereof, or if the activity of a polypeptide encoded by a nucleic acid molecule comprising the nucleic acid SEQ ID NO. 20346, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 20346 or polypeptide SEQ ID NO. 20347, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 71 to 1094-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a CBL-interacting protein kinase, or if the activity of the polypeptide At2g34180, preferably represented by SEQ ID NO. 20579, or a homolog or fragment thereof, or if the activity of a polypeptide encoded by a nucleic acid molecule comprising the nucleic acid SEQ ID NO. 20578, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 20578 or polypeptide SEQ ID NO. 20579, respectively, or a homolog or a fragment thereof, is increased or generated, or if the activity CBL-interacting protein kinase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 37 to 136-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a delta-1-pyrroline 5-carboxylase synthetase, or if the activity of the polypeptide At2g39800, preferably represented by SEQ ID NO. 21009, or a homolog or fragment thereof, or if the activity of a polypeptide encoded by a nucleic acid molecule comprising the nucleic acid SEQ ID NO. 21008, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 21008 or polypeptide SEQ ID NO. 21009, respectively, or a homolog or a fragment thereof, is increased or generated, or if the activity delta-1-pyrroline 5-carboxylase synthetase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 106 to 4073-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a phosphatidylinositol 3- and 4-kinase family protein, or if the activity of the polypeptide At2g46500, preferably represented by SEQ ID NO. 21107, or a homolog or fragment thereof, or if the activity of a polypeptide encoded by a nucleic acid molecule comprising the nucleic acid SEQ ID NO. 21106, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 21106 or polypeptide SEQ ID NO. 21107, respectively, or a homolog or a fragment thereof, is increased or generated, or if the activity phosphatidylinositol 3- and 4-kinase family protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 44 to 135-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a glutaredoxin, or if the activity of the polypeptide At2g47880, preferably represented by SEQ ID NO. 21160, or a homolog or fragment thereof, or if the activity of a polypeptide encoded by a nucleic acid molecule comprising the nucleic acid SEQ ID NO. 21159, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 21159 or polypeptide SEQ ID NO. 21160, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 253 to 1026-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a glutaredoxin, or if the activity of the polypeptide At2g47880, preferably represented by SEQ ID NO. 21160, or a homolog or fragment thereof, or if the activity of a polypeptide encoded by a nucleic acid molecule comprising the nucleic acid SEQ ID NO. 21159, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 21159 or polypeptide SEQ ID NO. 21160, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 90 to 179-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a glutaredoxin, or if the activity of the polypeptide At2g47880, preferably represented by SEQ ID NO. 21160, or a homolog or fragment thereof, or if the activity of a polypeptide encoded by a nucleic acid molecule comprising the nucleic acid SEQ ID NO. 21159, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 21159 or polypeptide SEQ ID NO. 21160, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 49 to 717-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a pyruvate kinase, or if the activity of the polypeptide At3g04050, preferably represented by SEQ ID NO. 21498, or a homolog or fragment thereof, or if the activity of a polypeptide encoded by a nucleic acid molecule comprising the nucleic acid SEQ ID NO. 21497, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 21497 or polypeptide SEQ ID NO. 21498, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line discloses in column 7 glutamate. For example, an increase of the glutamate of at least 1 percent, particularly in a range of 36 to 311-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a pyruvate kinase, or if the activity of the polypeptide At3g04050, preferably represented by SEQ ID NO. 21498, or a homolog or fragment thereof, or if the activity of a polypeptide encoded by a nucleic acid molecule comprising the nucleic acid SEQ ID NO. 21497, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 21497 or polypeptide SEQ ID NO. 21498, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line discloses in column 7 proline. For example, an increase of the proline of at least 1 percent, particularly in a range of 33 to 272-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a ankyrin repeat family protein, or if the activity of the polypeptide At3g04710, preferably represented by SEQ ID NO. 21903, or a homolog or fragment thereof, or if the activity of a polypeptide encoded by a nucleic acid molecule comprising the nucleic acid SEQ ID NO. 21902, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 21902 or polypeptide SEQ ID NO. 21903, respectively, or a homolog or a fragment thereof, is increased or generated, or if the activity ankyrin repeat family protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 35 to 230-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a protein phosphatase, or if the activity of the polypeptide At3g06270, preferably represented by SEQ ID NO. 22016, or a homolog or fragment thereof, or if the activity of a polypeptide encoded by a nucleic acid molecule comprising the nucleic acid SEQ ID NO. 22015, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 22015 or polypeptide SEQ ID NO. 22016, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 36 to 99-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a thioredoxin family protein, or if the activity of the polypeptide At3g08710, preferably represented by SEQ ID NO. 22250, or a homolog or fragment thereof, or if the activity of a polypeptide encoded by a nucleic acid molecule comprising the nucleic acid SEQ ID NO. 22249, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 22249 or polypeptide SEQ ID NO. 22250, respectively, or a homolog or a fragment thereof, is increased or generated, or if the activity thioredoxin family protein is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 56 to 513-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a thioredoxin family protein, or if the activity of the polypeptide At3g08710, preferably represented by SEQ ID NO. 22250, or a homolog or fragment thereof, or if the activity of a polypeptide encoded by a nucleic acid molecule comprising the nucleic acid SEQ ID NO. 22249, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 22249 or polypeptide SEQ ID NO. 22250, respectively, or a homolog or a fragment thereof, is increased or generated, or if the activity thioredoxin family protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 45 to 167-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a thioredoxin family protein, or if the activity of the polypeptide At3g08710, preferably represented by SEQ ID NO. 22250, or a homolog or fragment thereof, or if the activity of a polypeptide encoded by a nucleic acid molecule comprising the nucleic acid SEQ ID NO. 22249, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 22249 or polypeptide SEQ ID NO. 22250, respectively, or a homolog or a fragment thereof, is increased or generated, or if the activity thioredoxin family protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 76 to 558-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a thioredoxin family protein, or if the activity of the polypeptide At3g08710, preferably represented by SEQ ID NO. 22250, or a homolog or fragment thereof, or if the activity of a polypeptide encoded by a nucleic acid molecule comprising the nucleic acid SEQ ID NO. 22249, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 22249 or polypeptide SEQ ID NO. 22250, respectively, or a homolog or a fragment thereof, is increased or generated, or if the activity thioredoxin family protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 70 to 169-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a harpin-induced family protein, or if the activity of the polypeptide At3g11650, preferably represented by SEQ ID NO. 22612, or a homolog or fragment thereof, or if the activity of a polypeptide encoded by a nucleic acid molecule comprising the nucleic acid SEQ ID NO. 22611, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 22611 or polypeptide SEQ ID NO. 22612, respectively, or a homolog or a fragment thereof, is increased or generated, or if the activity harpin-induced family protein is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 46 to 397-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a harpin-induced family protein, or if the activity of the polypeptide At3g11650, preferably represented by SEQ ID NO. 22612, or a homolog or fragment thereof, or if the activity of a polypeptide encoded by a nucleic acid molecule comprising the nucleic acid SEQ ID NO. 22611, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 22611 or polypeptide SEQ ID NO. 22612, respectively, or a homolog or a fragment thereof, is increased or generated, or if the activity harpin-induced family protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 44 to 124-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a harpin-induced family protein, or if the activity of the polypeptide At3g11650, preferably represented by SEQ ID NO. 22612, or a homolog or fragment thereof, or if the activity of a polypeptide encoded by a nucleic acid molecule comprising the nucleic acid SEQ ID NO. 22611, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 22611 or polypeptide SEQ ID NO. 22612, respectively, or a homolog or a fragment thereof, is increased or generated, or if the activity harpin-induced family protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 50 to 129-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a DNA binding protein, or if the activity of the polypeptide At3g14230, preferably represented by SEQ ID NO. 22700, or a homolog or fragment thereof, or if the activity of a polypeptide encoded by a nucleic acid molecule comprising the nucleic acid SEQ ID NO. 22699, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 22699 or polypeptide SEQ ID NO. 22700, respectively, or a homolog or a fragment thereof, is increased or generated, or if the activity DNA binding protein is increased or generated in a non-human organism, like a microorganism or a plant cell, plant or part thereof, especially with plastidic localization, whereby the respective line discloses in column 7 proline. For example, an increase of the proline of at least 1 percent, particularly in a range of 53 to 142-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a DNA mismatch repair protein, or if the activity of the polypeptide At3g18524, preferably represented by SEQ ID NO. 22833, or a homolog or fragment thereof, or if the activity of a polypeptide encoded by a nucleic acid molecule comprising the nucleic acid SEQ ID NO. 22832, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 22832 or polypeptide SEQ ID NO. 22833, respectively, or a homolog or a fragment thereof, is increased or generated, or if the activity DNA mismatch repair protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 38 to 75-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a CCAAT-binding transcription factor, or if the activity of the polypeptide At3g20910, preferably represented by SEQ ID NO. 22922, or a homolog or fragment thereof, or if the activity of a polypeptide encoded by a nucleic acid molecule comprising the nucleic acid SEQ ID NO. 22921, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 22921 or polypeptide SEQ ID NO. 22922, respectively, or a homolog or a fragment thereof, is increased or generated, or if the activity CCAAT-binding transcription factor is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 61 to 204-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a protein kinase, or if the activity of the polypeptide At3g23000, preferably represented by SEQ ID NO. 1816, or a homolog or fragment thereof, or if the activity of a polypeptide encoded by a nucleic acid molecule comprising the nucleic acid SEQ ID NO. 1815, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 1815 or polypeptide SEQ ID NO. 1816, respectively, or a homolog or a fragment thereof, is increased or generated, or if the activity protein kinase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 79 to 288-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a glucose-6-phosphate 1-dehydrogenase, or if the activity of the polypeptide At3g27300, preferably represented by SEQ ID NO. 23003, or a homolog or fragment thereof, or if the activity of a polypeptide encoded by a nucleic acid molecule comprising the nucleic acid SEQ ID NO. 23002, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 23002 or polypeptide SEQ ID NO. 23003, respectively, or a homolog or a fragment thereof, is increased or generated, or if the activity glucose-6-phosphate 1-dehydrogenase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 38 to 123-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a monthiol glutaredoxin, or if the activity of the polypeptide At3g62930, preferably represented by SEQ ID NO. 2574, or a homolog or fragment thereof, or if the activity of a polypeptide encoded by a nucleic acid molecule comprising the nucleic acid SEQ ID NO. 2573, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 2573 or polypeptide SEQ ID NO. 2574, respectively, or a homolog or a fragment thereof, is increased or generated, or if the activity monthiol glutaredoxin is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 39 to 132-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a monthiol glutaredoxin, or if the activity of the polypeptide At3g62930, preferably represented by SEQ ID NO. 2574, or a homolog or fragment thereof, or if the activity of a polypeptide encoded by a nucleic acid molecule comprising the nucleic acid SEQ ID NO. 2573, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 2573 or polypeptide SEQ ID NO. 2574, respectively, or a homolog or a fragment thereof, is increased or generated, or if the activity monthiol glutaredoxin is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 51 to 277-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 33 to 89-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 127 to 250-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 38 to 164-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 44 to 116-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a monothiol glutaredoxin, or if the activity of the polypeptide At4g15670, preferably represented by SEQ ID NO. 3280, or a homolog or fragment thereof, or if the activity of a polypeptide encoded by a nucleic acid molecule comprising the nucleic acid SEQ ID NO. 3279, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 3279 or polypeptide SEQ ID NO. 3280, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 77 to 165-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a monothiol glutaredoxin, or if the activity of the polypeptide At4g15690, preferably represented by SEQ ID NO. 23845, or a homolog or fragment thereof, or if the activity of a polypeptide encoded by a nucleic acid molecule comprising the nucleic acid SEQ ID NO. 23844, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 23844 or polypeptide SEQ ID NO. 23845, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 47 to 145-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a monothiol glutaredoxin, or if the activity of the polypeptide At4g15690, preferably represented by SEQ ID NO. 23845, or a homolog or fragment thereof, or if the activity of a polypeptide encoded by a nucleic acid molecule comprising the nucleic acid SEQ ID NO. 23844, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 23844 or polypeptide SEQ ID NO. 23845, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 53 to 270-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a monothiol glutaredoxin, or if the activity of the polypeptide At4g15690, preferably represented by SEQ ID NO. 23845, or a homolog or fragment thereof, or if the activity of a polypeptide encoded by a nucleic acid molecule comprising the nucleic acid SEQ ID NO. 23844, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 23844 or polypeptide SEQ ID NO. 23845, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 44 to 156-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a monothiol glutaredoxin, or if the activity of the polypeptide At4g15700, preferably represented by SEQ ID NO. 3655, or a homolog or fragment thereof, or if the activity of a polypeptide encoded by a nucleic acid molecule comprising the nucleic acid SEQ ID NO. 3654, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 3654 or polypeptide SEQ ID NO. 3655, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 55 to 267-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a heat shock transcription factor, or if the activity of the polypeptide At4g18880, preferably represented by SEQ ID NO. 24233, or a homolog or fragment thereof, or if the activity of a polypeptide encoded by a nucleic acid molecule comprising the nucleic acid SEQ ID NO. 24232, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 24232 or polypeptide SEQ ID NO. 24233, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 46 to 216-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a At4g32480-protein, or if the activity of the polypeptide At4g32480, preferably represented by SEQ ID NO. 4041, or a homolog or fragment thereof, or if the activity of a polypeptide encoded by a nucleic acid molecule comprising the nucleic acid SEQ ID NO. 4040, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 4040 or polypeptide SEQ ID NO. 4041, respectively, or a homolog or a fragment thereof, is increased or generated, or if the activity At4g32480-protein is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 52 to 215-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a At4g32480-protein, or if the activity of the polypeptide At4g32480, preferably represented by SEQ ID NO. 4041, or a homolog or fragment thereof, or if the activity of a polypeptide encoded by a nucleic acid molecule comprising the nucleic acid SEQ ID NO. 4040, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 4040 or polypeptide SEQ ID NO. 4041, respectively, or a homolog or a fragment thereof, is increased or generated, or if the activity At4g32480-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 301 to 4258-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a At4g32480-protein, or if the activity of the polypeptide At4g32480, preferably represented by SEQ ID NO. 4041, or a homolog or fragment thereof, or if the activity of a polypeptide encoded by a nucleic acid molecule comprising the nucleic acid SEQ ID NO. 4040, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 4040 or polypeptide SEQ ID NO. 4041, respectively, or a homolog or a fragment thereof, is increased or generated, or if the activity At4g32480-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 45 to 111-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a glutaredoxin, or if the activity of the polypeptide At4g33040, preferably represented by SEQ ID NO. 4103, or a homolog or fragment thereof, or if the activity of a polypeptide encoded by a nucleic acid molecule comprising the nucleic acid SEQ ID NO. 4102, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 4102 or polypeptide SEQ ID NO. 4103, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 46 to 104-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a cyclin D, or if the activity of the polypeptide At4g34160, preferably represented by SEQ ID NO. 24312, or a homolog or fragment thereof, or if the activity of a polypeptide encoded by a nucleic acid molecule comprising the nucleic acid SEQ ID NO. 24311, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 24311 or polypeptide SEQ ID NO. 24312, respectively, or a homolog or a fragment thereof, is increased or generated, or if the activity cyclin D is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 90 to 236-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a cyclin D, or if the activity of the polypeptide At4g34160, preferably represented by SEQ ID NO. 24312, or a homolog or fragment thereof, or if the activity of a polypeptide encoded by a nucleic acid molecule comprising the nucleic acid SEQ ID NO. 24311, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 24311 or polypeptide SEQ ID NO. 24312, respectively, or a homolog or a fragment thereof, is increased or generated, or if the activity cyclin D is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 51 to 313-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a cyclin D, or if the activity of the polypeptide At4g34160, preferably represented by SEQ ID NO. 24312, or a homolog or fragment thereof, or if the activity of a polypeptide encoded by a nucleic acid molecule comprising the nucleic acid SEQ ID NO. 24311, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 24311 or polypeptide SEQ ID NO. 24312, respectively, or a homolog or a fragment thereof, is increased or generated, or if the activity cyclin D is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 49 to 772-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 58 to 499-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 126 to 329-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a heat shock transcription factor, or if the activity of the polypeptide At5g03720, preferably represented by SEQ ID NO. 24439, or a homolog or fragment thereof, or if the activity of a polypeptide encoded by a nucleic acid molecule comprising the nucleic acid SEQ ID NO. 24438, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 24438 or polypeptide SEQ ID NO. 24439, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 67 to 186-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a gibberellin 20-oxidase, or if the activity of the polypeptide At5g07200, preferably represented by SEQ ID NO. 24493, or a homolog or fragment thereof, or if the activity of a polypeptide encoded by a nucleic acid molecule comprising the nucleic acid SEQ ID NO. 24492, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 24492 or polypeptide SEQ ID NO. 24493, respectively, or a homolog or a fragment thereof, is increased or generated, or if the activity gibberellin 20-oxidase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 44 to 99-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a integral membrane transporter family protein, or if the activity of the polypeptide At5g10820, preferably represented by SEQ ID NO. 25223, or a homolog or fragment thereof, or if the activity of a polypeptide encoded by a nucleic acid molecule comprising the nucleic acid SEQ ID NO. 25222, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 25222 or polypeptide SEQ ID NO. 25223, respectively, or a homolog or a fragment thereof, is increased or generated, or if the activity integral membrane transporter family protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 34 to 69-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a integral membrane transporter family protein, or if the activity of the polypeptide At5g10820, preferably represented by SEQ ID NO. 25223, or a homolog or fragment thereof, or if the activity of a polypeptide encoded by a nucleic acid molecule comprising the nucleic acid SEQ ID NO. 25222, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 25222 or polypeptide SEQ ID NO. 25223, respectively, or a homolog or a fragment thereof, is increased or generated, or if the activity integral membrane transporter family protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 45 to 139-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a At5g16650-protein, or if the activity of the polypeptide At5g16650, preferably represented by SEQ ID NO. 25284, or a homolog or fragment thereof, or if the activity of a polypeptide encoded by a nucleic acid molecule comprising the nucleic acid SEQ ID NO. 25283, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 25283 or polypeptide SEQ ID NO. 25284, respectively, or a homolog or a fragment thereof, is increased or generated, or if the activity At5g16650-protein is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 104 to 361-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a At5g16650-protein, or if the activity of the polypeptide At5g16650, preferably represented by SEQ ID NO. 25284, or a homolog or fragment thereof, or if the activity of a polypeptide encoded by a nucleic acid molecule comprising the nucleic acid SEQ ID NO. 25283, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 25283 or polypeptide SEQ ID NO. 25284, respectively, or a homolog or a fragment thereof, is increased or generated, or if the activity At5g16650-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 48 to 238-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a At5g16650-protein, or if the activity of the polypeptide At5g16650, preferably represented by SEQ ID NO. 25284, or a homolog or fragment thereof, or if the activity of a polypeptide encoded by a nucleic acid molecule comprising the nucleic acid SEQ ID NO. 25283, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 25283 or polypeptide SEQ ID NO. 25284, respectively, or a homolog or a fragment thereof, is increased or generated, or if the activity At5g16650-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 41 to 177-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a monothiol glutaredoxin, or if the activity of the polypeptide At5g18600, preferably represented by SEQ ID NO. 4905, or a homolog or fragment thereof, or if the activity of a polypeptide encoded by a nucleic acid molecule comprising the nucleic acid SEQ ID NO. 4904, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 4904 or polypeptide SEQ ID NO. 4905, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 82 to 254-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a translation initiation factor subunit, or if the activity of the polypeptide At5g27640, preferably represented by SEQ ID NO. 25345, or a homolog or fragment thereof, or if the activity of a polypeptide encoded by a nucleic acid molecule comprising the nucleic acid SEQ ID NO. 25344, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 25344 or polypeptide SEQ ID NO. 25345, respectively, or a homolog or a fragment thereof, is increased or generated, or if the activity translation initiation factor subunit is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 67 to 139-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a transcription factor, or if the activity of the polypeptide At5g39760, preferably represented by SEQ ID NO. 25429, or a homolog or fragment thereof, or if the activity of a polypeptide encoded by a nucleic acid molecule comprising the nucleic acid SEQ ID NO. 25428, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 25428 or polypeptide SEQ ID NO. 25429, respectively, or a homolog or a fragment thereof, is increased or generated, or if the activity transcription factor is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 40 to 78-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a transcription factor, or if the activity of the polypeptide At5g39760, preferably represented by SEQ ID NO. 25429, or a homolog or fragment thereof, or if the activity of a polypeptide encoded by a nucleic acid molecule comprising the nucleic acid SEQ ID NO. 25428, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 25428 or polypeptide SEQ ID NO. 25429, respectively, or a homolog or a fragment thereof, is increased or generated, or if the activity transcription factor is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 32 to 275-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a protein phosphatase, or if the activity of the polypeptide At5g57050, preferably represented by SEQ ID NO. 5319, or a homolog or fragment thereof, or if the activity of a polypeptide encoded by a nucleic acid molecule comprising the nucleic acid SEQ ID NO. 5318, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 5318 or polypeptide SEQ ID NO. 5319, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 34 to 63-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a protein phosphatase, or if the activity of the polypeptide At5g59220, preferably represented by SEQ ID NO. 25499, or a homolog or fragment thereof, or if the activity of a polypeptide encoded by a nucleic acid molecule comprising the nucleic acid SEQ ID NO. 25498, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 25498 or polypeptide SEQ ID NO. 25499, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 32 to 52-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a zinc finger protein, or if the activity of the polypeptide At5g64920, preferably represented by SEQ ID NO. 5494, or a homolog or fragment thereof, or if the activity of a polypeptide encoded by a nucleic acid molecule comprising the nucleic acid SEQ ID NO. 5493, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 5493 or polypeptide SEQ ID NO. 5494, respectively, or a homolog or a fragment thereof, is increased or generated, or if the activity zinc finger protein is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 53 to 291-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a zinc finger protein, or if the activity of the polypeptide At5g64920, preferably represented by SEQ ID NO. 5494, or a homolog or fragment thereof, or if the activity of a polypeptide encoded by a nucleic acid molecule comprising the nucleic acid SEQ ID NO. 5493, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 5493 or polypeptide SEQ ID NO. 5494, respectively, or a homolog or a fragment thereof, is increased or generated, or if the activity zinc finger protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 52 to 199-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a transcriptional regulator, or if the activity of the polypeptide AvinDRAFT_{—}1045, preferably represented by SEQ ID NO. 25677, or a homolog or fragment thereof, or if the activity of a polypeptide encoded by a nucleic acid molecule comprising the nucleic acid SEQ ID NO. 25676, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 25676 or polypeptide SEQ ID NO. 25677, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 55 to 90-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a adenylylsulfate kinase, or if the activity of the polypeptide AvinDRAFT_{—}1398, preferably represented by SEQ ID NO. 25781, or a homolog or fragment thereof, or if the activity of a polypeptide encoded by a nucleic acid molecule comprising the nucleic acid SEQ ID NO. 25780, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 25780 or polypeptide SEQ ID NO. 25781, respectively, or a homolog or a fragment thereof, is increased or generated, or if the activity adenylylsulfate kinase is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 42 to 44-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a adenylylsulfate kinase, or if the activity of the polypeptide AvinDRAFT_{—}1398, preferably represented by SEQ ID NO. 25781, or a homolog or fragment thereof, or if the activity of a polypeptide encoded by a nucleic acid molecule comprising the nucleic acid SEQ ID NO. 25780, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 25780 or polypeptide SEQ ID NO. 25781, respectively, or a homolog or a fragment thereof, is increased or generated, or if the activity adenylylsulfate kinase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 56 to 79-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a malic enzyme, or if the activity of the polypeptide AvinDRAFT_{—}1495, preferably represented by SEQ ID NO. 5558, or a homolog or fragment thereof, or if the activity of a polypeptide encoded by a nucleic acid molecule comprising the nucleic acid SEQ ID NO. 5557, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 5557 or polypeptide SEQ ID NO. 5558, respectively, or a homolog or a fragment thereof, is increased or generated, or if the activity malic enzyme is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 54 to 292-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a malic enzyme, or if the activity of the polypeptide AvinDRAFT_{—}1495, preferably represented by SEQ ID NO. 5558, or a homolog or fragment thereof, or if the activity of a polypeptide encoded by a nucleic acid molecule comprising the nucleic acid SEQ ID NO. 5557, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 5557 or polypeptide SEQ ID NO. 5558, respectively, or a homolog or a fragment thereof, is increased or generated, or if the activity malic enzyme is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 111 to 227-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a glycosyl transferase, or if the activity of the polypeptide AvinDRAFT_{—}1534, preferably represented by SEQ ID NO. 26121, or a homolog or fragment thereof, or if the activity of a polypeptide encoded by a nucleic acid molecule comprising the nucleic acid SEQ ID NO. 26120, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 26120 or polypeptide SEQ ID NO. 26121, respectively, or a homolog or a fragment thereof, is increased or generated, or if the activity glycosyl transferase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 44 to 119-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a sec-independent protein translocase, or if the activity of the polypeptide AvinDRAFT_{—}1624, preferably represented by SEQ ID NO. 26197, or a homolog or fragment thereof, or if the activity of a polypeptide encoded by a nucleic acid molecule comprising the nucleic acid SEQ ID NO. 26196, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 26196 or polypeptide SEQ ID NO. 26197, respectively, or a homolog or a fragment thereof, is increased or generated, or if the activity sec-independent protein translocase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 35 to 78-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a pyruvate kinase, or if the activity of the polypeptide AvinDRAFT_{—}1806, preferably represented by SEQ ID NO. 26435, or a homolog or fragment thereof, or if the activity of a polypeptide encoded by a nucleic acid molecule comprising the nucleic acid SEQ ID NO. 26434, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 26434 or polypeptide SEQ ID NO. 26435, respectively, or a homolog or a fragment thereof, is increased or generated, or if the activity pyruvate kinase is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 76 to 95-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a betahydroxylase, or if the activity of the polypeptide AvinDRAFT_{—}2091, preferably represented by SEQ ID NO. 6041, or a homolog or fragment thereof, or if the activity of a polypeptide encoded by a nucleic acid molecule comprising the nucleic acid SEQ ID NO. 6040, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 6040 or polypeptide SEQ ID NO. 6041, respectively, or a homolog or a fragment thereof, is increased or generated, or if the activity beta-hydroxylase is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 64 to 530-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a elongation factor Tu, or if the activity of the polypeptide AvinDRAFT_{—}2344, preferably represented by SEQ ID NO. 27022, or a homolog or fragment thereof, or if the activity of a polypeptide encoded by a nucleic acid molecule comprising the nucleic acid SEQ ID NO. 27021, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 27021 or polypeptide SEQ ID NO. 27022, respectively, or a homolog or a fragment thereof, is increased or generated, or if the activity elongation factor Tu is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 115 to 495-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a elongation factor Tu, or if the activity of the polypeptide AvinDRAFT_{—}2344, preferably represented by SEQ ID NO. 27022, or a homolog or fragment thereof, or if the activity of a polypeptide encoded by a nucleic acid molecule comprising the nucleic acid SEQ ID NO. 27021, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 27021 or polypeptide SEQ ID NO. 27022, respectively, or a homolog or a fragment thereof, is increased or generated, or if the activity elongation factor Tu is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 44 to 145-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a elongation factor Tu, or if the activity of the polypeptide AvinDRAFT_{—}2344, preferably represented by SEQ ID NO. 27022, or a homolog or fragment thereof, or if the activity of a polypeptide encoded by a nucleic acid molecule comprising the nucleic acid SEQ ID NO. 27021, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 27021 or polypeptide SEQ ID NO. 27022, respectively, or a homolog or a fragment thereof, is increased or generated, or if the activity elongation factor Tu is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 76 to 770-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a ABC transporter permease protein, or if the activity of the polypeptide AvinDRAFT_{—}2521, preferably represented by SEQ ID NO. 27883, or a homolog or fragment thereof, or if the activity of a polypeptide encoded by a nucleic acid molecule comprising the nucleic acid SEQ ID NO. 27882, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 27882 or polypeptide SEQ ID NO. 27883, respectively, or a homolog or a fragment thereof, is increased or generated, or if the activity ABC transporter permease protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 58 to 197-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a ABC transporter permease protein, or if the activity of the polypeptide AvinDRAFT_{—}2521, preferably represented by SEQ ID NO. 27883, or a homolog or fragment thereof, or if the activity of a polypeptide encoded by a nucleic acid molecule comprising the nucleic acid SEQ ID NO. 27882, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 27882 or polypeptide SEQ ID NO. 27883, respectively, or a homolog or a fragment thereof, is increased or generated, or if the activity ABC transporter permease protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 39 to 466-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a acyl-CoA synthase, or if the activity of the polypeptide AvinDRAFT_{—}2754, preferably represented by SEQ ID NO. 28041, or a homolog or fragment thereof, or if the activity of a polypeptide encoded by a nucleic acid molecule comprising the nucleic acid SEQ ID NO. 28040, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 28040 or polypeptide SEQ ID NO. 28041, respectively, or a homolog or a fragment thereof, is increased or generated, or if the activity acyl-CoA synthase is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 73 to 227-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a acyl-CoA synthase, or if the activity of the polypeptide AvinDRAFT_{—}2754, preferably represented by SEQ ID NO. 28041, or a homolog or fragment thereof, or if the activity of a polypeptide encoded by a nucleic acid molecule comprising the nucleic acid SEQ ID NO. 28040, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 28040 or polypeptide SEQ ID NO. 28041, respectively, or a homolog or a fragment thereof, is increased or generated, or if the activity acyl-CoA synthase is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 38 to 297-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a acyl-CoA synthase, or if the activity of the polypeptide AvinDRAFT_{—}2754, preferably represented by SEQ ID NO. 28041, or a homolog or fragment thereof, or if the activity of a polypeptide encoded by a nucleic acid molecule comprising the nucleic acid SEQ ID NO. 28040, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 28040 or polypeptide SEQ ID NO. 28041, respectively, or a homolog or a fragment thereof, is increased or generated, or if the activity acyl-CoA synthase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 80 to 466-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a acyl-CoA synthase, or if the activity of the polypeptide AvinDRAFT_{—}2754, preferably represented by SEQ ID NO. 28041, or a homolog or fragment thereof, or if the activity of a polypeptide encoded by a nucleic acid molecule comprising the nucleic acid SEQ ID NO. 28040, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 28040 or polypeptide SEQ ID NO. 28041, respectively, or a homolog or a fragment thereof, is increased or generated, or if the activity acyl-CoA synthase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 36 to 170-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a 2-oxoglutarate dehydrogenase E1 subunit, or if the activity of the polypeptide AvinDRAFT_{—}3028, preferably represented by SEQ ID NO. 6076, or a homolog or fragment thereof, or if the activity of a polypeptide encoded by a nucleic acid molecule comprising the nucleic acid SEQ ID NO. 6075, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 6075 or polypeptide SEQ ID NO. 6076, respectively, or a homolog or a fragment thereof, is increased or generated, or if the activity 2-oxoglutarate dehydrogenase E1 subunit is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 64 to 185-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a enoyl-CoA hydratase, or if the activity of the polypeptide AvinDRAFT_{—}3159, preferably represented by SEQ ID NO. 28739, or a homolog or fragment thereof, or if the activity of a polypeptide encoded by a nucleic acid molecule comprising the nucleic acid SEQ ID NO. 28738, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 28738 or polypeptide SEQ ID NO. 28739, respectively, or a homolog or a fragment thereof, is increased or generated, or if the activity enoyl-CoA hydratase is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 64 to 144-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a enoyl-CoA hydratase, or if the activity of the polypeptide AvinDRAFT_{—}3159, preferably represented by SEQ ID NO. 28739, or a homolog or fragment thereof, or if the activity of a polypeptide encoded by a nucleic acid molecule comprising the nucleic acid SEQ ID NO. 28738, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 28738 or polypeptide SEQ ID NO. 28739, respectively, or a homolog or a fragment thereof, is increased or generated, or if the activity enoyl-CoA hydratase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 63 to 531-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a transcriptional regulator, or if the activity of the polypeptide AvinDRAFT_{—}3186, preferably represented by SEQ ID NO. 29247, or a homolog or fragment thereof, or if the activity of a polypeptide encoded by a nucleic acid molecule comprising the nucleic acid SEQ ID NO. 29246, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 29246 or polypeptide SEQ ID NO. 29247, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 38 to 91-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a transcriptional regulator, or if the activity of the polypeptide AvinDRAFT_{—}3209, preferably represented by SEQ ID NO. 29287, or a homolog or fragment thereof, or if the activity of a polypeptide encoded by a nucleic acid molecule comprising the nucleic acid SEQ ID NO. 29286, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 29286 or polypeptide SEQ ID NO. 29287, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 105 to 202-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a acyltransferase, or if the activity of the polypeptide AvinDRAFT_{—}3250, preferably represented by SEQ ID NO. 29398, or a homolog or fragment thereof, or if the activity of a polypeptide encoded by a nucleic acid molecule comprising the nucleic acid SEQ ID NO. 29397, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 29397 or polypeptide SEQ ID NO. 29398, respectively, or a homolog or a fragment thereof, is increased or generated, or if the activity acyltransferase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 50 to 405-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a oxidoreductase, or if the activity of the polypeptide AvinDRAFT_{—}3253, preferably represented by SEQ ID NO. 29501, or a homolog or fragment thereof, or if the activity of a polypeptide encoded by a nucleic acid molecule comprising the nucleic acid SEQ ID NO. 29500, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 29500 or polypeptide SEQ ID NO. 29501, respectively, or a homolog or a fragment thereof, is increased or generated, or if the activity oxidoreductase is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 34 to 63-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a oxidoreductase, or if the activity of the polypeptide AvinDRAFT_{—}3253, preferably represented by SEQ ID NO. 29501, or a homolog or fragment thereof, or if the activity of a polypeptide encoded by a nucleic acid molecule comprising the nucleic acid SEQ ID NO. 29500, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 29500 or polypeptide SEQ ID NO. 29501, respectively, or a homolog or a fragment thereof, is increased or generated, or if the activity oxidoreductase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 53 to 128-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a lysyl-tRNA synthetase, or if the activity of the polypeptide AvinDRAFT_{—}3556, preferably represented by SEQ ID NO. 30040, or a homolog or fragment thereof, or if the activity of a polypeptide encoded by a nucleic acid molecule comprising the nucleic acid SEQ ID NO. 30039, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 30039 or polypeptide SEQ ID NO. 30040, respectively, or a homolog or a fragment thereof, is increased or generated, or if the activity lysyl-tRNA synthetase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 37 to 65-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a 30S ribosomal protein, or if the activity of the polypeptide AvinDRAFT_{—}3587, preferably represented by SEQ ID NO. 30465, or a homolog or fragment thereof, or if the activity of a polypeptide encoded by a nucleic acid molecule comprising the nucleic acid SEQ ID NO. 30464, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 30464 or polypeptide SEQ ID NO. 30465, respectively, or a homolog or a fragment thereof, is increased or generated, or if the activity 30S ribosomal protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 36 to 71-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a CTP synthase, or if the activity of the polypeptide AvinDRAFT_{—}3605, preferably represented by SEQ ID NO. 31027, or a homolog or fragment thereof, or if the activity of a polypeptide encoded by a nucleic acid molecule comprising the nucleic acid SEQ ID NO. 31026, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 31026 or polypeptide SEQ ID NO. 31027, respectively, or a homolog or a fragment thereof, is increased or generated, or if the activity CTP synthase is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 35 to 57-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a CTP synthase, or if the activity of the polypeptide AvinDRAFT_{—}3605, preferably represented by SEQ ID NO. 31027, or a homolog or fragment thereof, or if the activity of a polypeptide encoded by a nucleic acid molecule comprising the nucleic acid SEQ ID NO. 31026, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 31026 or polypeptide SEQ ID NO. 31027, respectively, or a homolog or a fragment thereof, is increased or generated, or if the activity CTP synthase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 60 to 275-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a glucose-1-phosphate cytidylyltransferase, or if the activity of the polypeptide AvinDRAFT_{—}4384, preferably represented by SEQ ID NO. 31718, or a homolog or fragment thereof, or if the activity of a polypeptide encoded by a nucleic acid molecule comprising the nucleic acid SEQ ID NO. 31717, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 31717 or polypeptide SEQ ID NO. 31718, respectively, or a homolog or a fragment thereof, is increased or generated, or if the activity glucose-1-phosphate cytidylyltransferase is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 35 to 98-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a glucose-1-phosphate cytidylyltransferase, or if the activity of the polypeptide AvinDRAFT_{—}4384, preferably represented by SEQ ID NO. 31718, or a homolog or fragment thereof, or if the activity of a polypeptide encoded by a nucleic acid molecule comprising the nucleic acid SEQ ID NO. 31717, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 31717 or polypeptide SEQ ID NO. 31718, respectively, or a homolog or a fragment thereof, is increased or generated, or if the activity glucose-1-phosphate cytidylyltransferase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 75 to 196-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a glucose-1-phosphate cytidylyltransferase, or if the activity of the polypeptide AvinDRAFT_{—}4384, preferably represented by SEQ ID NO. 31718, or a homolog or fragment thereof, or if the activity of a polypeptide encoded by a nucleic acid molecule comprising the nucleic acid SEQ ID NO. 31717, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 31717 or polypeptide SEQ ID NO. 31718, respectively, or a homolog or a fragment thereof, is increased or generated, or if the activity glucose-1-phosphate cytidylyltransferase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 49 to 109-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a aminotransferase, or if the activity of the polypeptide AvinDRAFT_{—}4562, preferably represented by SEQ ID NO. 31927, or a homolog or fragment thereof, or if the activity of a polypeptide encoded by a nucleic acid molecule comprising the nucleic acid SEQ ID NO. 31926, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 31926 or polypeptide SEQ ID NO. 31927, respectively, or a homolog or a fragment thereof, is increased or generated, or if the activity aminotransferase is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 58 to 219-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a hydrolase, or if the activity of the polypeptide AvinDRAFT_{—}5103, preferably represented by SEQ ID NO. 6511, or a homolog or fragment thereof, or if the activity of a polypeptide encoded by a nucleic acid molecule comprising the nucleic acid SEQ ID NO. 6510, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 6510 or polypeptide SEQ ID NO. 6511, respectively, or a homolog or a fragment thereof, is increased or generated, or if the activity hydrolase is increased or generated in a non-human organism, like a microorganism or a plant cell, plant or part thereof, especially with non-targeted localization, whereby the respective line discloses in column 7 proline. For example, an increase of the proline of at least 1 percent, partitularly in a range of 66 to 289-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a glutamate-ammonia-ligase, or if the activity of the polypeptide AvinDRAFT_{—}5246, preferably represented by SEQ ID NO. 32038, or a homolog or fragment thereof, or if the activity of a polypeptide encoded by a nucleic acid molecule comprising the nucleic acid SEQ ID NO. 32037, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 32037 or polypeptide SEQ ID NO. 32038, respectively, or a homolog or a fragment thereof, is increased or generated, or if the activity glutamate-ammonia-ligase is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 71 to 419-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a glutamate-ammonia-ligase, or if the activity of the polypeptide AvinDRAFT_{—}5246, preferably represented by SEQ ID NO. 32038, or a homolog or fragment thereof, or if the activity of a polypeptide encoded by a nucleic acid molecule comprising the nucleic acid SEQ ID NO. 32037, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 32037 or polypeptide SEQ ID NO. 32038, respectively, or a homolog or a fragment thereof, is increased or generated, or if the activity glutamate-ammonia-ligase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 70 to 363-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a glutamate-ammonia-ligase, or if the activity of the polypeptide AvinDRAFT_{—}5246, preferably represented by SEQ ID NO. 32038, or a homolog or fragment thereof, or if the activity of a polypeptide encoded by a nucleic acid molecule comprising the nucleic acid SEQ ID NO. 32037, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 32037 or polypeptide SEQ ID NO. 32038, respectively, or a homolog or a fragment thereof, is increased or generated, or if the activity glutamate-ammonia-ligase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 69 to 218-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a fumarylacetoacetate hydrolase, or if the activity of the polypeptide AvinDRAFT_{—}5292, preferably represented by SEQ ID NO. 32309, or a homolog or fragment thereof, or if the activity of a polypeptide encoded by a nucleic acid molecule comprising the nucleic acid SEQ ID NO. 32308, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 32308 or polypeptide SEQ ID NO. 32309, respectively, or a homolog or a fragment thereof, is increased or generated, or if the activity fumarylacetoacetate hydrolase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 47 to 172-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a HesB/YadR/YfhF family protein, or if the activity of the polypeptide AvinDRAFT_{—}5467, preferably represented by SEQ ID NO. 32649, or a homolog or fragment thereof, or if the activity of a polypeptide encoded by a nucleic acid molecule comprising the nucleic acid SEQ ID NO. 32648, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 32648 or polypeptide SEQ ID NO. 32649, respectively, or a homolog or a fragment thereof, is increased or generated, or if the activity HesB/YadR/YfhF family protein is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 49 to 171-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a HesB/YadR/YfhF family protein, or if the activity of the polypeptide AvinDRAFT_{—}5467, preferably represented by SEQ ID NO. 32649, or a homolog or fragment thereof, or if the activity of a polypeptide encoded by a nucleic acid molecule comprising the nucleic acid SEQ ID NO. 32648, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 32648 or polypeptide SEQ ID NO. 32649, respectively, or a homolog or a fragment thereof, is increased or generated, or if the activity HesB/YadR/YfhF family protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 45 to 123-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a HesB/YadR/YfhF family protein, or if the activity of the polypeptide AvinDRAFT_{—}5467, preferably represented by SEQ ID NO. 32649, or a homolog or fragment thereof, or if the activity of a polypeptide encoded by a nucleic acid molecule comprising the nucleic acid SEQ ID NO. 32648, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 32648 or polypeptide SEQ ID NO. 32649, respectively, or a homolog or a fragment thereof, is increased or generated, or if the activity HesB/YadR/YfhF family protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 50 to 132-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a L-aspartate oxidase, or if the activity of the polypeptide AvinDRAFT_{—}5644, preferably represented by SEQ ID NO. 33086, or a homolog or fragment thereof, or if the activity of a polypeptide encoded by a nucleic acid molecule comprising the nucleic acid SEQ ID NO. 33085, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 33085 or polypeptide SEQ ID NO. 33086, respectively, or a homolog or a fragment thereof, is increased or generated, or if the activity L-aspartate oxidase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 51 to 152-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a oxidoreductase, or if the activity of the polypeptide AvinDRAFT_{—}5651, preferably represented by SEQ ID NO. 33458, or a homolog or fragment thereof, or if the activity of a polypeptide encoded by a nucleic acid molecule comprising the nucleic acid SEQ ID NO. 33457, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 33457 or polypeptide SEQ ID NO. 33458, respectively, or a homolog or a fragment thereof, is increased or generated, or if the activity oxidoreductase is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 62 to 368-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a oxidoreductase, or if the activity of the polypeptide AvinDRAFT_{—}5651, preferably represented by SEQ ID NO. 33458, or a homolog or fragment thereof, or if the activity of a polypeptide encoded by a nucleic acid molecule comprising the nucleic acid SEQ ID NO. 33457, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 33457 or polypeptide SEQ ID NO. 33458, respectively, or a homolog or a fragment thereof, is increased or generated, or if the activity oxidoreductase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 92 to 226-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a Chaperone protein CIpB, or if the activity of the polypeptide AvinDRAFT_{—}6093, preferably represented by SEQ ID NO. 33597, or a homolog or fragment thereof, or if the activity of a polypeptide encoded by a nucleic acid molecule comprising the nucleic acid SEQ ID NO. 33596, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 33596 or polypeptide SEQ ID NO. 33597, respectively, or a homolog or a fragment thereof, is increased or generated, or if the activity Chaperone protein CIpB is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 42 to 112-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a purine nucleoside phosphorylase, or if the activity of the polypeptide AvinDRAFT_{—}6700, preferably represented by SEQ ID NO. 34045, or a homolog or fragment thereof, or if the activity of a polypeptide encoded by a nucleic acid molecule comprising the nucleic acid SEQ ID NO. 34044, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 34044 or polypeptide SEQ ID NO. 34045, respectively, or a homolog or a fragment thereof, is increased or generated, or if the activity purine nucleoside phosphorylase is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 99 to 186-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a purine nucleoside phosphorylase, or if the activity of the polypeptide AvinDRAFT_{—}6700, preferably represented by SEQ ID NO. 34045, or a homolog or fragment thereof, or if the activity of a polypeptide encoded by a nucleic acid molecule comprising the nucleic acid SEQ ID NO. 34044, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 34044 or polypeptide SEQ ID NO. 34045, respectively, or a homolog or a fragment thereof, is increased or generated, or if the activity purine nucleoside phosphorylase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 45 to 168-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a purine nucleoside phosphorylase, or if the activity of the polypeptide AvinDRAFT_{—}6700, preferably represented by SEQ ID NO. 34045, or a homolog or fragment thereof, or if the activity of a polypeptide encoded by a nucleic acid molecule comprising the nucleic acid SEQ ID NO. 34044, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 34044 or polypeptide SEQ ID NO. 34045, respectively, or a homolog or a fragment thereof, is increased or generated, or if the activity purine nucleoside phosphorylase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 47 to 112-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a transcriptional regulator protein, or if the activity of the polypeptide AvinDRAFT_{—}6864, preferably represented by SEQ ID NO. 34205, or a homolog or fragment thereof, or if the activity of a polypeptide encoded by a nucleic acid molecule comprising the nucleic acid SEQ ID NO. 34204, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 34204 or polypeptide SEQ ID NO. 34205, respectively, or a homolog or a fragment thereof, is increased or generated, or if the activity transcriptional regulator protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 47 to 75-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a AX653549-protein, or if the activity of the polypeptide AX653549, preferably represented by SEQ ID NO. 34302, or a homolog or fragment thereof, or if the activity of a polypeptide encoded by a nucleic acid molecule comprising the nucleic acid SEQ ID NO. 34301, preferably the coding region thereof, or a homolog or fragment thereof, e.g. derived from Oryza sativa, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 34301 or polypeptide SEQ ID NO. 34302, respectively, or a homolog or a fragment thereof, is increased or generated, or if the activity AX653549-protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 47 to 57-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a AX653549-protein, or if the activity of the polypeptide AX653549, preferably represented by SEQ ID NO. 34302, or a homolog or fragment thereof, or if the activity of a polypeptide encoded by a nucleic acid molecule comprising the nucleic acid SEQ ID NO. 34301, preferably the coding region thereof, or a homolog or fragment thereof, e.g. derived from Oryza sativa, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 34301 or polypeptide SEQ ID NO. 34302, respectively, or a homolog or a fragment thereof, is increased or generated, or if the activity AX653549-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 58 to 355-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a AY087308-protein, or if the activity of the polypeptide AY087308, preferably represented by SEQ ID NO. 34603, or a homolog or fragment thereof, or if the activity of a polypeptide encoded by a nucleic acid molecule comprising the nucleic acid SEQ ID NO. 34602, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 34602 or polypeptide SEQ ID NO. 34603, respectively, or a homolog or a fragment thereof, is increased or generated, or if the activity AY087308-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 53 to 114-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a threonine synthase, or if the activity of the polypeptide B0004, preferably represented by SEQ ID NO. 34890, or a homolog or fragment thereof, or if the activity of a polypeptide encoded by a nucleic acid molecule comprising the nucleic acid SEQ ID NO. 34889, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 34889 or polypeptide SEQ ID NO. 34890, respectively, or a homolog or a fragment thereof, is increased or generated, or if the activity threonine synthase is increased or generated in a non-human organism, like a microorganism or a plant cell, plant or part thereof, especially with plastidic localization, whereby the respective line discloses in column 7 arginine. For example, an increase of the arginine of at least 1 percent, particularly in a range of 56 to 2425-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a threonine synthase, or if the activity of the polypeptide B0004, preferably represented by SEQ ID NO. 34890, or a homolog or fragment thereof, or if the activity of a polypeptide encoded by a nucleic acid molecule comprising the nucleic acid SEQ ID NO. 34889, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 34889 or polypeptide SEQ ID NO. 34890, respectively, or a homolog or a fragment thereof, is increased or generated, or if the activity threonine synthase is increased or generated in a non-human organism, like a microorganism or a plant cell, plant or part thereof, especially with plastidic localization, whereby the respective line discloses in column 7 glutamine. For example, an increase of the glutamine of at least 1 percent, particularly in a range of 49 to 4802-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a threonine synthase, or if the activity of the polypeptide B0004, preferably represented by SEQ ID NO. 34890, or a homolog or fragment thereof, or if the activity of a polypeptide encoded by a nucleic acid molecule comprising the nucleic acid SEQ ID NO. 34889, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 34889 or polypeptide SEQ ID NO. 34890, respectively, or a homolog or a fragment thereof, is increased or generated, or if the activity threonine synthase is increased or generated in a non-human organism, like a microorganism or a plant cell, plant or part thereof, especially with plastidic localization, whereby the respective line discloses in column 7 proline. For example, an increase of the proline of at least 1 percent, particularly in a range of 40 to 1754-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a L-ribulose-5-phosphate 4-epimerase, or if the activity of the polypeptide B0061, preferably represented by SEQ ID NO. 35205, or a homolog or fragment thereof, or if the activity of a polypeptide encoded by a nucleic acid molecule comprising the nucleic acid SEQ ID NO. 35204, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 35204 or polypeptide SEQ ID NO. 35205, respectively, or a homolog or a fragment thereof, is increased or generated, or if the activity L-ribulose-5-phosphate 4-epimerase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 137 to 1181-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a dihydrolipoamide acetyltransferase, or if the activity of the polypeptide B0115, preferably represented by SEQ ID NO. 35367, or a homolog or fragment thereof, or if the activity of a polypeptide encoded by a nucleic acid molecule comprising the nucleic acid SEQ ID NO. 35366, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 35366 or polypeptide SEQ ID NO. 35367, respectively, or a homolog or a fragment thereof, is increased or generated, or if the activity dihydrolipoamide acetyltransferase is increased or generated in a non-human organism, like a microorganism or a plant cell, plant or part thereof, especially with plastidic localization, whereby the respective line discloses in column 7 glutamine. For example, an increase of the glutamine of at least 1 percent, particularly in a range of 67 to 86-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a glucose dehydrogenase, or if the activity of the polypeptide B0124, preferably represented by SEQ ID NO. 35483, or a homolog or fragment thereof, or if the activity of a polypeptide encoded by a nucleic acid molecule comprising the nucleic acid SEQ ID NO. 35482, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 35482 or polypeptide SEQ ID NO. 35483, respectively, or a homolog or a fragment thereof, is increased or generated, or if the activity glucose dehydrogenase is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 53 to 395-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a glucose dehydrogenase, or if the activity of the polypeptide B0124, preferably represented by SEQ ID NO. 35483, or a homolog or fragment thereof, or if the activity of a polypeptide encoded by a nucleic acid molecule comprising the nucleic acid SEQ ID NO. 35482, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 35482 or polypeptide SEQ ID NO. 35483, respectively, or a homolog or a fragment thereof, is increased or generated, or if the activity glucose dehydrogenase is increased or generated in a non-human organism, like a microorganism or a plant cell, plant or part thereof, especially with plastidic localization, whereby the respective line discloses in column 7 proline. For example, an increase of the proline of at least 1 percent, particularly in a range of 47 to 389-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a glucose dehydrogenase, or if the activity of the polypeptide B0124, preferably represented by SEQ ID NO. 35483, or a homolog or fragment thereof, or if the activity of a polypeptide encoded by a nucleic acid molecule comprising the nucleic acid SEQ ID NO. 35482, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 35482 or polypeptide SEQ ID NO. 35483, respectively, or a homolog or a fragment thereof, is increased or generated, or if the activity glucose dehydrogenase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 47 to 124-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a serine protease, or if the activity of the polypeptide B0161, preferably represented by SEQ ID NO. 7082, or a homolog or fragment thereof, or if the activity of a polypeptide encoded by a nucleic acid molecule comprising the nucleic acid SEQ ID NO. 7081, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 7081 or polypeptide SEQ ID NO. 7082, respectively, or a homolog or a fragment thereof, is increased or generated, or if the activity serine protease is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 100 to 233-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a acyl-CoA dehydrogenase, or if the activity of the polypeptide B0221, preferably represented by SEQ ID NO. 35591, or a homolog or fragment thereof, or if the activity of a polypeptide encoded by a nucleic acid molecule comprising the nucleic acid SEQ ID NO. 35590, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 35590 or polypeptide SEQ ID NO. 35591, respectively, or a homolog or a fragment thereof, is increased or generated, or if the activity acyl-CoA dehydrogenase is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 61 to 202-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a acyl-CoA dehydrogenase, or if the activity of the polypeptide B0221, preferably represented by SEQ ID NO. 35591, or a homolog or fragment thereof, or if the activity of a polypeptide encoded by a nucleic acid molecule comprising the nucleic acid SEQ ID NO. 35590, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 35590 or polypeptide SEQ ID NO. 35591, respectively, or a homolog or a fragment thereof, is increased or generated, or if the activity acyl-CoA dehydrogenase is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 54 to 78-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a beta-galactosidase, or if the activity of the polypeptide B0344, preferably represented by SEQ ID NO. 35734, or a homolog or fragment thereof, or if the activity of a polypeptide encoded by a nucleic acid molecule comprising the nucleic acid SEQ ID NO. 35733, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 35733 or polypeptide SEQ ID NO. 35734, respectively, or a homolog or a fragment thereof, is increased or generated, or if the activity beta-galactosidase is increased or generated in a non-human organism, like a microorganism or a plant cell, plant or part thereof, especially with plastidic localization, whereby the respective line discloses in column 7 proline. For example, an increase of the proline of at least 1 percent, particularly in a range of 118 to 227-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a ATP-binding component of a transport system, or if the activity of the polypeptide B0449, preferably represented by SEQ ID NO. 7334, or a homolog or fragment thereof, or if the activity of a polypeptide encoded by a nucleic acid molecule comprising the nucleic acid SEQ ID NO. 7333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 7333 or polypeptide SEQ ID NO. 7334, respectively, or a homolog or a fragment thereof, is increased or generated, or if the activity ATP-binding component of a transport system is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 51 to 961-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a b0456-protein, or if the activity of the polypeptide B0456, preferably represented by SEQ ID NO. 35876, or a homolog or fragment thereof, or if the activity of a polypeptide encoded by a nucleic acid molecule comprising the nucleic acid SEQ ID NO. 35875, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 35875 or polypeptide SEQ ID NO. 35876, respectively, or a homolog or a fragment thereof, is increased or generated, or if the activity b0456-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 75 to 121-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a membrane transport protein, or if the activity of the polypeptide B0486, preferably represented by SEQ ID NO. 7687, or a homolog or fragment thereof, or if the activity of a polypeptide encoded by a nucleic acid molecule comprising the nucleic acid SEQ ID NO. 7686, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 7686 or polypeptide SEQ ID NO. 7687, respectively, or a homolog or a fragment thereof, is increased or generated, or if the activity membrane transport protein is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 67 to 213-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a membrane transport protein, or if the activity of the polypeptide B0486, preferably represented by SEQ ID NO. 7687, or a homolog or fragment thereof, or if the activity of a polypeptide encoded by a nucleic acid molecule comprising the nucleic acid SEQ ID NO. 7686, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 7686 or polypeptide SEQ ID NO. 7687, respectively, or a homolog or a fragment thereof, is increased or generated, or if the activity membrane transport protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 33 to 57-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a b0518-protein, or if the activity of the polypeptide B0518, preferably represented by SEQ ID NO. 35937, or a homolog or fragment thereof, or if the activity of a polypeptide encoded by a nucleic acid molecule comprising the nucleic acid SEQ ID NO. 35936, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 35936 or polypeptide SEQ ID NO. 35937, respectively, or a homolog or a fragment thereof, is increased or generated, or if the activity b0518-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 48 to 190-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a isochorismate synthase, or if the activity of the polypeptide B0593, preferably represented by SEQ ID NO. 35968, or a homolog or fragment thereof, or if the activity of a polypeptide encoded by a nucleic acid molecule comprising the nucleic acid SEQ ID NO. 35967, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 35967 or polypeptide SEQ ID NO. 35968, respectively, or a homolog or a fragment thereof, is increased or generated, or if the activity isochorismate synthase is increased or generated in a non-human organism, like a microorganism or a plant cell, plant or part thereof, especially with plastidic localization, whereby the respective line discloses in column 7 arginine. For example, an increase of the arginine of at least 1 percent, particularly in a range of 137 to 775-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a isochorismate synthase, or if the activity of the polypeptide B0593, preferably represented by SEQ ID NO. 35968, or a homolog or fragment thereof, or if the activity of a polypeptide encoded by a nucleic acid molecule comprising the nucleic acid SEQ ID NO. 35967, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 35967 or polypeptide SEQ ID NO. 35968, respectively, or a homolog or a fragment thereof, is increased or generated, or if the activity isochorismate synthase is increased or generated in a non-human organism, like a microorganism or a plant cell, plant or part thereof, especially with plastidic localization, whereby the respective line discloses in column 7 glutamate. For example, an increase of the glutamate of at least 1 percent, particularly in a range of 36 to 80-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a isochorismate synthase, or if the activity of the polypeptide B0593, preferably represented by SEQ ID NO. 35968, or a homolog or fragment thereof, or if the activity of a polypeptide encoded by a nucleic acid molecule comprising the nucleic acid SEQ ID NO. 35967, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 35967 or polypeptide SEQ ID NO. 35968, respectively, or a homolog or a fragment thereof, is increased or generated, or if the activity isochorismate synthase is increased or generated in a non-human organism, like a microorganism or a plant cell, plant or part thereof, especially with plastidic localization, whereby the respective line discloses in column 7 glutamine. For example, an increase of the glutamine of at least 1 percent, particularly in a range of 34 to 132-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a isochorismate synthase, or if the activity of the polypeptide B0593, preferably represented by SEQ ID NO. 35968, or a homolog or fragment thereof, or if the activity of a polypeptide encoded by a nucleic acid molecule comprising the nucleic acid SEQ ID NO. 35967, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 35967 or polypeptide SEQ ID NO. 35968, respectively, or a homolog or a fragment thereof, is increased or generated, or if the activity isochorismate synthase is increased or generated in a non-human organism, like a microorganism or a plant cell, plant or part thereof, especially with plastidic localization, whereby the respective line discloses in column 7 proline. For example, an increase of the proline of at least 1 percent, particularly in a range of 71 to 148-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a zinc transporter, or if the activity of the polypeptide B0752, preferably represented by SEQ ID NO. 36115, or a homolog or fragment thereof, or if the activity of a polypeptide encoded by a nucleic acid molecule comprising the nucleic acid SEQ ID NO. 36114, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 36114 or polypeptide SEQ ID NO. 36115, respectively, or a homolog or a fragment thereof, is increased or generated, or if the activity zinc transporter is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 60 to 126-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a asparaginase, or if the activity of the polypeptide B0828, preferably represented by SEQ ID NO. 36300, or a homolog or fragment thereof, or if the activity of a polypeptide encoded by a nucleic acid molecule comprising the nucleic acid SEQ ID NO. 36299, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 36299 or polypeptide SEQ ID NO. 36300, respectively, or a homolog or a fragment thereof, is increased or generated, or if the activity asparaginase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 35 to 80-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a Leucyl/phenylalanyl-tRNA-protein transferase, or if the activity of the polypeptide B0885, preferably represented by SEQ ID NO. 36490, or a homolog or fragment thereof, or if the activity of a polypeptide encoded by a nucleic acid molecule comprising the nucleic acid SEQ ID NO. 36489, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 36489 or polypeptide SEQ ID NO. 36490, respectively, or a homolog or a fragment thereof, is increased or generated, or if the activity Leucyl/phenylalanyl-tRNA-protein transferase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 29 to 80-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a Leucyl/phenylalanyl-tRNA-protein transferase, or if the activity of the polypeptide B0885, preferably represented by SEQ ID NO. 36490, or a homolog or fragment thereof, or if the activity of a polypeptide encoded by a nucleic acid molecule comprising the nucleic acid SEQ ID NO. 36489, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 36489 or polypeptide SEQ ID NO. 36490, respectively, or a homolog or a fragment thereof, is increased or generated, or if the activity Leucyl/phenylalanyl-tRNA-protein transferase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 50 to 181-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a major facilitator superfamily transporter protein, or if the activity of the polypeptide B0898, preferably represented by SEQ ID NO. 7918, or a homolog or fragment thereof, or if the activity of a polypeptide encoded by a nucleic acid molecule comprising the nucleic acid SEQ ID NO. 7917, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 7917 or polypeptide SEQ ID NO. 7918, respectively, or a homolog or a fragment thereof, is increased or generated, or if the activity major facilitator superfamily transporter protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 39 to 138-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a major facilitator superfamily transporter protein, or if the activity of the polypeptide B0898, preferably represented by SEQ ID NO. 7918, or a homolog or fragment thereof, or if the activity of a polypeptide encoded by a nucleic acid molecule comprising the nucleic acid SEQ ID NO. 7917, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 7917 or polypeptide SEQ ID NO. 7918, respectively, or a homolog or a fragment thereof, is increased or generated, or if the activity major facilitator superfamily transporter protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 68 to 8167-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a DNA helicase IV, or if the activity of the polypeptide B0962, preferably represented by SEQ ID NO. 36624, or a homolog or fragment thereof, or if the activity of a polypeptide encoded by a nucleic acid molecule comprising the nucleic acid SEQ ID NO. 36623, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 36623 or polypeptide SEQ ID NO. 36624, respectively, or a homolog or a fragment thereof, is increased or generated, or if the activity DNA helicase IV is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 114 to 475-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a methylglyoxal synthase, or if the activity of the polypeptide B0963, preferably represented by SEQ ID NO. 36671, or a homolog or fragment thereof, or if the activity of a polypeptide encoded by a nucleic acid molecule comprising the nucleic acid SEQ ID NO. 36670, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 36670 or polypeptide SEQ ID NO. 36671, respectively, or a homolog or a fragment thereof, is increased or generated, or if the activity methylglyoxal synthase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 40 to 332-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a phosphoanhydride phosphorylase, or if the activity of the polypeptide B0980, preferably represented by SEQ ID NO. 36810, or a homolog or fragment thereof, or if the activity of a polypeptide encoded by a nucleic acid molecule comprising the nucleic acid SEQ ID NO. 36809, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 36809 or polypeptide SEQ ID NO. 36810, respectively, or a homolog or a fragment thereof, is increased or generated, or if the activity phosphoanhydride phosphorylase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 233 to 304-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a b1003-protein, or if the activity of the polypeptide B1003, preferably represented by SEQ ID NO. 7942, or a homolog or fragment thereof, or if the activity of a polypeptide encoded by a nucleic acid molecule comprising the nucleic acid SEQ ID NO. 7941, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 7941 or polypeptide SEQ ID NO. 7942, respectively, or a homolog or a fragment thereof, is increased or generated, or if the activity b1003-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 71 to 2938-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a b1003-protein, or if the activity of the polypeptide B1003, preferably represented by SEQ ID NO. 7942, or a homolog or fragment thereof, or if the activity of a polypeptide encoded by a nucleic acid molecule comprising the nucleic acid SEQ ID NO. 7941, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 7941 or polypeptide SEQ ID NO. 7942, respectively, or a homolog or a fragment thereof, is increased or generated, or if the activity b1003-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 56 to 1315-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a lipoprotein precursor, or if the activity of the polypeptide B1023, preferably represented by SEQ ID NO. 36881, or a homolog or fragment thereof, or if the activity of a polypeptide encoded by a nucleic acid molecule comprising the nucleic acid SEQ ID NO. 36880, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 36880 or polypeptide SEQ ID NO. 36881, respectively, or a homolog or a fragment thereof, is increased or generated, or if the activity lipoprotein precursor is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 53 to 554-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a b1024-protein, or if the activity of the polypeptide B1024, preferably represented by SEQ ID NO. 36908, or a homolog or fragment thereof, or if the activity of a polypeptide encoded by a nucleic acid molecule comprising the nucleic acid SEQ ID NO. 36907, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 36907 or polypeptide SEQ ID NO. 36908, respectively, or a homolog or a fragment thereof, is increased or generated, or if the activity b1024-protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 38 to 55-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a b1108-protein, or if the activity of the polypeptide B1108, preferably represented by SEQ ID NO. 36938, or a homolog or fragment thereof, or if the activity of a polypeptide encoded by a nucleic acid molecule comprising the nucleic acid SEQ ID NO. 36937, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 36937 or polypeptide SEQ ID NO. 36938, respectively, or a homolog or a fragment thereof, is increased or generated, or if the activity b1108-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 61 to 491-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a b1108-protein, or if the activity of the polypeptide B1108, preferably represented by SEQ ID NO. 36938, or a homolog or fragment thereof, or if the activity of a polypeptide encoded by a nucleic acid molecule comprising the nucleic acid SEQ ID NO. 36937, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 36937 or polypeptide SEQ ID NO. 36938, respectively, or a homolog or a fragment thereof, is increased or generated, or if the activity b1108-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 34 to 225-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a isocitrate dehydrogenase, or if the activity of the polypeptide B1136, preferably represented by SEQ ID NO. 36972, or a homolog or fragment thereof, or if the activity of a polypeptide encoded by a nucleic acid molecule comprising the nucleic acid SEQ ID NO. 36971, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 36971 or polypeptide SEQ ID NO. 36972, respectively, or a homolog or a fragment thereof, is increased or generated, or if the activity isocitrate dehydrogenase is increased or generated in a non-human organism, like a microorganism or a plant cell, plant or part thereof, especially with plastidic localization, whereby the respective line discloses in column 7 proline. For example, an increase of the proline of at least 1 percent, particularly in a range of 47 to 549-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a b1137-protein, or if the activity of the polypeptide B1137, preferably represented by SEQ ID NO. 37391, or a homolog or fragment thereof, or if the activity of a polypeptide encoded by a nucleic acid molecule comprising the nucleic acid SEQ ID NO. 37390, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 37390 or polypeptide SEQ ID NO. 37391, respectively, or a homolog or a fragment thereof, is increased or generated, or if the activity b1137-protein is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 85 to 748-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a b1137-protein, or if the activity of the polypeptide B1137, preferably represented by SEQ ID NO. 37391,or a homolog or fragment thereof, or if the activity of a polypeptide encoded by a nucleic acid molecule comprising the nucleic acid SEQ ID NO. 37390, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 37390 or polypeptide SEQ ID NO. 37391, respectively, or a homolog or a fragment thereof, is increased or generated, or if the activity b1137-protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 48 to 69-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a b1137-protein, or if the activity of the polypeptide B1137, preferably represented by SEQ ID NO. 37391, or a homolog or fragment thereof, or if the activity of a polypeptide encoded by a nucleic acid molecule comprising the nucleic acid SEQ ID NO. 37390, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 37390 or polypeptide SEQ ID NO. 37391, respectively, or a homolog or a fragment thereof, is increased or generated, or if the activity b1137-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 40 to 169-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a b1163-protein, or if the activity of the polypeptide B1163, preferably represented by SEQ ID NO. 37395, or a homolog or fragment thereof, or if the activity of a polypeptide encoded by a nucleic acid molecule comprising the nucleic acid SEQ ID NO. 37394, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 37394 or polypeptide SEQ ID NO. 37395, respectively, or a homolog or a fragment thereof, is increased or generated, or if the activity b1163-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 30 to 36-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a sodium/proton antiporter, or if the activity of the polypeptide B1186, preferably represented by SEQ ID NO. 37401, or a homolog or fragment thereof, or if the activity of a polypeptide encoded by a nucleic acid molecule comprising the nucleic acid SEQ ID NO. 37400, preferably the coding region thereof, or a homolog or fragment thereof, e.g. derived from Escherichia coli K12, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 37400 or polypeptide SEQ ID NO. 37401, respectively, or a homolog or a fragment thereof, is increased or generated, or if the activity sodium/proton antiporter is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 36 to 164-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a membrane protein, or if the activity of the polypeptide B1255, preferably represented by SEQ ID NO. 37484, or a homolog or fragment thereof, or if the activity of a polypeptide encoded by a nucleic acid molecule comprising the nucleic acid SEQ ID NO. 37483, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 37483 or polypeptide SEQ ID NO. 37484, respectively, or a homolog or a fragment thereof, is increased or generated, or if the activity membrane protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 42 to 80-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a membrane protein, or if the activity of the polypeptide B1255, preferably represented by SEQ ID NO. 37484, or a homolog or fragment thereof, or if the activity of a polypeptide encoded by a nucleic acid molecule comprising the nucleic acid SEQ ID NO. 37483, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 37483 or polypeptide SEQ ID NO. 37484, respectively, or a homolog or a fragment thereof, is increased or generated, or if the activity membrane protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 39 to 520-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a b1259-protein, or if the activity of the polypeptide B1259, preferably represented by SEQ ID NO. 37504, or a homolog or fragment thereof, or if the activity of a polypeptide encoded by a nucleic acid molecule comprising the nucleic acid SEQ ID NO. 37503, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 37503 or polypeptide SEQ ID NO. 37504, respectively, or a homolog or a fragment thereof, is increased or generated, or if the activity b1259-protein is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 66 to 105-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a b1259-protein, or if the activity of the polypeptide B1259, preferably represented by SEQ ID NO. 37504, or a homolog or fragment thereof, or if the activity of a polypeptide encoded by a nucleic acid molecule comprising the nucleic acid SEQ ID NO. 37503, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 37503 or polypeptide SEQ ID NO. 37504, respectively, or a homolog or a fragment thereof, is increased or generated, or if the activity b1259-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 58 to 291-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a anthranilate synthase component II, or if the activity of the polypeptide B1263, preferably represented by SEQ ID NO. 37540, or a homolog or fragment thereof, or if the activity of a polypeptide encoded by a nucleic acid molecule comprising the nucleic acid SEQ ID NO. 37539, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 37539 or polypeptide SEQ ID NO. 37540, respectively, or a homolog or a fragment thereof, is increased or generated, or if the activity anthranilate synthase component II is increased or generated in a non-human organism, like a microorganism or a plant cell, plant or part thereof, especially with plastidic localization, whereby the respective line discloses in column 7 glutamine. For example, an increase of the glutamine of at least 1 percent, particularly in a range of 68 to 176-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a b1280-protein, or if the activity of the polypeptide B1280, preferably represented by SEQ ID NO. 37574, or a homolog or fragment thereof, or if the activity of a polypeptide encoded by a nucleic acid molecule comprising the nucleic acid SEQ ID NO. 37573, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 37573 or polypeptide SEQ ID NO. 37574, respectively, or a homolog or a fragment thereof, is increased or generated, or if the activity b1280-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 94 to 179-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a glutamine synthetase, or if the activity of the polypeptide B1297, preferably represented by SEQ ID NO. 37659, or a homolog or fragment thereof, or if the activity of a polypeptide encoded by a nucleic acid molecule comprising the nucleic acid SEQ ID NO. 37658, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 37658 or polypeptide SEQ ID NO. 37659, respectively, or a homolog or a fragment thereof, is increased or generated, or if the activity glutamine synthetase is increased or generated in a non-human organism, like a microorganism or a plant cell, plant or part thereof, especially with plastidic localization, whereby the respective line discloses in column 7 glutamine. For example, an increase of the glutamine of at least 1 percent, particularly in a range of 70 to 412-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a aldehyde dehydrogenase, or if the activity of the polypeptide B1300, preferably represented by SEQ ID NO. 37808, or a homolog or fragment thereof, or if the activity of a polypeptide encoded by a nucleic acid molecule comprising the nucleic acid SEQ ID NO. 37807, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 37807 or polypeptide SEQ ID NO. 37808, respectively, or a homolog or a fragment thereof, is increased or generated, or if the activity aldehyde dehydrogenase is increased or generated in a non-human organism, like a microorganism or a plant cell, plant or part thereof, especially with non-targeted localization, whereby the respective line discloses in column 7 glutamate of at least 1 percent, particularly in a range of 39 to 89-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a b1330-protein, or if the activity of the polypeptide B1330, preferably represented by SEQ ID NO. 38227, or a homolog or fragment thereof, or if the activity of a polypeptide encoded by a nucleic acid molecule comprising the nucleic acid SEQ ID NO. 38226, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 38226 or polypeptide SEQ ID NO. 38227, respectively, or a homolog or a fragment thereof, is increased or generated, or if the activity b1330-protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 50 to 97-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a b1330-protein, or if the activity of the polypeptide B1330, preferably represented by SEQ ID NO. 38227, or a homolog or fragment thereof, or if the activity of a polypeptide encoded by a nucleic acid molecule comprising the nucleic acid SEQ ID NO. 38226, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 38226 or polypeptide SEQ ID NO. 38227, respectively, or a homolog or a fragment thereof, is increased or generated, or if the activity b1330-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 28 to 102-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a lipoprotein precursor, or if the activity of the polypeptide B1431, preferably represented by SEQ ID NO. 38267, or a homolog or fragment thereof, or if the activity of a polypeptide encoded by a nucleic acid molecule comprising the nucleic acid SEQ ID NO. 38266, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 38266 or polypeptide SEQ ID NO. 38267, respectively, or a homolog or a fragment thereof, is increased or generated, or if the activity lipoprotein precursor is increased or generated in a non-human organism, like a microorganism or a plant cell, plant or part thereof, especially with plastidic localization, whereby the respective line discloses in column 7 glutamate. For example, an increase of the glutamate of at least 1 percent, particularly in a range of 33 to 52-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a lipoprotein precursor, or if the activity of the polypeptide B1431, preferably represented by SEQ ID NO. 38267, or a homolog or fragment thereof, or if the activity of a polypeptide encoded by a nucleic acid molecule comprising the nucleic acid SEQ ID NO. 38266, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 38266 or polypeptide SEQ ID NO. 38267, respectively, or a homolog or a fragment thereof, is increased or generated, or if the activity lipoprotein precursor is increased or generated in a non-human organism, like a microorganism or a plant cell, plant or part thereof, especially with plastidic localization, whereby the respective line discloses in column 7 glutamine. For example, an increase of the glutamine of at least 1 percent, particularly in a range of 63 to 83-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a b1445-protein, or if the activity of the polypeptide B1445, preferably represented by SEQ ID NO. 38290, or a homolog or fragment thereof, or if the activity of a polypeptide encoded by a nucleic acid molecule comprising the nucleic acid SEQ ID NO. 38289, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 38289 or polypeptide SEQ ID NO. 38290, respectively, or a homolog or a fragment thereof, is increased or generated, or if the activity b1445-protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 36 to 165-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a b1445-protein, or if the activity of the polypeptide B1445, preferably represented by SEQ ID NO. 38290, or a homolog or fragment thereof, or if the activity of a polypeptide encoded by a nucleic acid molecule comprising the nucleic acid SEQ ID NO. 38289, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 38289 or polypeptide SEQ ID NO. 38290, respectively, or a homolog or a fragment thereof, is increased or generated, or if the activity b1445-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 43 to 134-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a b1522-protein, or if the activity of the polypeptide B1522, preferably represented by SEQ ID NO. 7948, or a homolog or fragment thereof, or if the activity of a polypeptide encoded by a nucleic acid molecule comprising the nucleic acid SEQ ID NO. 7947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 7947 or polypeptide SEQ ID NO. 7948, respectively, or a homolog or a fragment thereof, is increased or generated, or if the activity b1522-protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 38 to 281-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a acid shock protein, or if the activity of the polypeptide B1597, preferably represented by SEQ ID NO. 38301, or a homolog or fragment thereof, or if the activity of a polypeptide encoded by a nucleic acid molecule comprising the nucleic acid SEQ ID NO. 38300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 38300 or polypeptide SEQ ID NO. 38301, respectively, or a homolog or a fragment thereof, is increased or generated, or if the activity acid shock protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 38 to 69-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a electron transport complex protein, or if the activity of the polypeptide B1627, preferably represented by SEQ ID NO. 38346, or a homolog or fragment thereof, or if the activity of a polypeptide encoded by a nucleic acid molecule comprising the nucleic acid SEQ ID NO. 38345, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 38345 or polypeptide SEQ ID NO. 38346, respectively, or a homolog or a fragment thereof, is increased or generated, or if the activity electron transport complex protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 43 to 239-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a protease, or if the activity of the polypeptide B1845, preferably represented by SEQ ID NO. 38574, or a homolog or fragment thereof, or if the activity of a polypeptide encoded by a nucleic acid molecule comprising the nucleic acid SEQ ID NO. 38573, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 38573 or polypeptide SEQ ID NO. 38574, respectively, or a homolog or a fragment thereof, is increased or generated, or if the activity protease is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 48 to 147-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a b1898-protein, or if the activity of the polypeptide B1898, preferably represented by SEQ ID NO. 38768, or a homolog or fragment thereof, or if the activity of a polypeptide encoded by a nucleic acid molecule comprising the nucleic acid SEQ ID NO. 38767, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 38767 or polypeptide SEQ ID NO. 38768, respectively, or a homolog or a fragment thereof, is increased or generated, or if the activity b1898-protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 40 to 45-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a transport protein, or if the activity of the polypeptide B1981, preferably represented by SEQ ID NO. 38900, or a homolog or fragment thereof, or if the activity of a polypeptide encoded by a nucleic acid molecule comprising the nucleic acid SEQ ID NO. 38899, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 38899 or polypeptide SEQ ID NO. 38900, respectively, or a homolog or a fragment thereof, is increased or generated, or if the activity transport protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 87 to 481-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a transport protein, or if the activity of the polypeptide B2063, preferably represented by SEQ ID NO. 38948, or a homolog or fragment thereof, or if the activity of a polypeptide encoded by a nucleic acid molecule comprising the nucleic acid SEQ ID NO. 38947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 38947 or polypeptide SEQ ID NO. 38948, respectively, or a homolog or a fragment thereof, is increased or generated, or if the activity transport protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 57 to 134-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a transport protein, or if the activity of the polypeptide B2063, preferably represented by SEQ ID NO. 38948, or a homolog or fragment thereof, or if the activity of a polypeptide encoded by a nucleic acid molecule comprising the nucleic acid SEQ ID NO. 38947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 38947 or polypeptide SEQ ID NO. 38948, respectively, or a homolog or a fragment thereof, is increased or generated, or if the activity transport protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 42 to 448-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a transport protein, or if the activity of the polypeptide B2063, preferably represented by SEQ ID NO. 38948, or a homolog or fragment thereof, or if the activity of a polypeptide encoded by a nucleic acid molecule comprising the nucleic acid SEQ ID NO. 38947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 38947 or polypeptide SEQ ID NO. 38948, respectively, or a homolog or a fragment thereof, is increased or generated, or if the activity transport protein is increased or generated in a non-human organism, like a microorganism or a plant cell, plant or part thereof, especially with non-targeted localization, whereby the respective line discloses in column 7 proline. For example, an increase of the prolinelocalization, whereby the respective line discloses in column 7 proline. For example, an increase of the proline of at least 1 percent, particularly in a range of 54 to 722-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a uridine/cytidine kinase, or if the activity of the polypeptide B2066, preferably represented by SEQ ID NO. 8938, or a homolog or fragment thereof, or if the activity of a polypeptide encoded by a nucleic acid molecule comprising the nucleic acid SEQ ID NO. 8937, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 8937 or polypeptide SEQ ID NO. 8938, respectively, or a homolog or a fragment thereof, is increased or generated, or if the activity uridine/cytidine kinase is increased or generated in a non-human organism, like a microorganism or a plant cell, plant or part thereof, especially with mitochondrial localization, whereby the respective line discloses in column 7 glutamine. For example, an increase of the glutamine of at least 1 percent, particularly in a range of 58 to 476-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a b2107-protein, or if the activity of the polypeptide B2107, preferably represented by SEQ ID NO. 39003, or a homolog or fragment thereof, or if the activity of a polypeptide encoded by a nucleic acid molecule comprising the nucleic acid SEQ ID NO. 39002, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 39002 or polypeptide SEQ ID NO. 39003, respectively, or a homolog or a fragment thereof, is increased or generated, or if the activity b2107-protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 35 to 185-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a b2121-protein, or if the activity of the polypeptide B2121, preferably represented by SEQ ID NO. 39014, or a homolog or fragment thereof, or if the activity of a polypeptide encoded by a nucleic acid molecule comprising the nucleic acid SEQ ID NO. 39013, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 39013 or polypeptide SEQ ID NO. 39014, respectively, or a homolog or a fragment thereof, is increased or generated, or if the activity b2121-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 70 to 156-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a ABC transporter permease protein, or if the activity of the polypeptide B2178, preferably represented by SEQ ID NO. 39041, or a homolog or fragment thereof, or if the activity of a polypeptide encoded by a nucleic acid molecule comprising the nucleic acid SEQ ID NO. 39040, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 39040 or polypeptide SEQ ID NO. 39041, respectively, or a homolog or a fragment thereof, is increased or generated, or if the activity ABC transporter permease protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 75 to 139-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a ABC transporter permease protein, or if the activity of the polypeptide B2178, preferably represented by SEQ ID NO. 39041, or a homolog or fragment thereof, or if the activity of a polypeptide encoded by a nucleic acid molecule comprising the nucleic acid SEQ ID NO. 39040, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 39040 or polypeptide SEQ ID NO. 39041, respectively, or a homolog or a fragment thereof, is increased or generated, or if the activity ABC transporter permease protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 43 to 350-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a ABC transporter permease protein, or if the activity of the polypeptide B2178, preferably represented by SEQ ID NO. 39041, or a homolog or fragment thereof, or if the activity of a polypeptide encoded by a nucleic acid molecule comprising the nucleic acid SEQ ID NO. 39040, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 39040 or polypeptide SEQ ID NO. 39041, respectively, or a homolog or a fragment thereof, is increased or generated, or if the activity ABC transporter permease protein is increased or generated in a non-human organism, like a microorganism or a plant cell, plant or part thereof, especially with non-targeted localization, whereby the respective line discloses in column 7 proline. For example, an increase of the prolinelocalization, whereby the respective line discloses in column 7 proline. For example, an increase of the proline of at least 1 percent, particularly in a range of 41 to 288 percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a NADH dehydrogenase I chain I, or if the activity of the polypeptide B2281, preferably represented by SEQ ID NO. 39121, or a homolog or fragment thereof, or if the activity of a polypeptide encoded by a nucleic acid molecule comprising the nucleic acid SEQ ID NO. 39120, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 39120 or polypeptide SEQ ID NO. 39121, respectively, or a homolog or a fragment thereof, is increased or generated, or if the activity NADH dehydrogenase I chain I is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 76 to 275-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a b2360-protein, or if the activity of the polypeptide B2360, preferably represented by SEQ ID NO. 39220, or a homolog or fragment thereof, or if the activity of a polypeptide encoded by a nucleic acid molecule comprising the nucleic acid SEQ ID NO. 39219, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 39219 or polypeptide SEQ ID NO. 39220, respectively, or a homolog or a fragment thereof, is increased or generated, or if the activity b2360-protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 42 to 121-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a b2399-protein, or if the activity of the polypeptide B2399, preferably represented by SEQ ID NO. 39238, or a homolog or fragment thereof, or if the activity of a polypeptide encoded by a nucleic acid molecule comprising the nucleic acid SEQ ID NO. 39237, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 39237 or polypeptide SEQ ID NO. 39238, respectively, or a homolog or a fragment thereof, is increased or generated, or if the activity b2399-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 146 to 3262-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a transcriptional regulator, or if the activity of the polypeptide B2405, preferably represented by SEQ ID NO. 39256, or a homolog or fragment thereof, or if the activity of a polypeptide encoded by a nucleic acid molecule comprising the nucleic acid SEQ ID NO. 39255, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 39255 or polypeptide SEQ ID NO. 39256, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 33 to 66-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a cysteine synthase A, or if the activity of the polypeptide B2414, preferably represented by SEQ ID NO. 39301, or a homolog or fragment thereof, or if the activity of a polypeptide encoded by a nucleic acid molecule comprising the nucleic acid SEQ ID NO. 39300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 39300 or polypeptide SEQ ID NO. 39301, respectively, or a homolog or a fragment thereof, is increased or generated, or if the activity cysteine synthase A is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 49 to 161-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a cysteine synthase A, or if the activity of the polypeptide B2414, preferably represented by SEQ ID NO. 39301, or a homolog or fragment thereof, or if the activity of a polypeptide encoded by a nucleic acid molecule comprising the nucleic acid SEQ ID NO. 39300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 39300 or polypeptide SEQ ID NO. 39301, respectively, or a homolog or a fragment thereof, is increased or generated, or if the activity cysteine synthase A is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 35 to 55-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a ethanolamine utilization protein, or if the activity of the polypeptide B2461, preferably represented by SEQ ID NO. 40300, or a homolog or fragment thereof, or if the activity of a polypeptide encoded by a nucleic acid molecule comprising the nucleic acid SEQ ID NO. 40299, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 40299 or polypeptide SEQ ID NO. 40300, respectively, or a homolog or a fragment thereof, is increased or generated, or if the activity ethanolamine utilization protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 40 to 408-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a b2474-protein, or if the activity of the polypeptide B2474, preferably represented by SEQ ID NO. 40330, or a homolog or fragment thereof, or if the activity of a polypeptide encoded by a nucleic acid molecule comprising the nucleic acid SEQ ID NO. 40329, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 40329 or polypeptide SEQ ID NO. 40330, respectively, or a homolog or a fragment thereof, is increased or generated, or if the activity b2474-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 42 to 89-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a b2513-protein, or if the activity of the polypeptide B2513, preferably represented by SEQ ID NO. 9168, or a homolog or fragment thereof, or if the activity of a polypeptide encoded by a nucleic acid molecule comprising the nucleic acid SEQ ID NO. 9167, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 9167 or polypeptide SEQ ID NO. 9168, respectively, or a homolog or a fragment thereof, is increased or generated, or if the activity b2513-protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 39 to 228-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a b2513-protein, or if the activity of the polypeptide B2513, preferably represented by SEQ ID NO. 9168, or a homolog or fragment thereof, or if the activity of a polypeptide encoded by a nucleic acid molecule comprising the nucleic acid SEQ ID NO. 9167, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 9167 or polypeptide SEQ ID NO. 9168, respectively, or a homolog or a fragment thereof, is increased or generated, or if the activity b2513-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 63 to 239-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a 2,3-dihydroxy-2,3-dihydrophenylpropionatedehydrogenase, or if the activity of the polypeptide B2541, preferably represented by SEQ ID NO. 40384, or a homolog or fragment thereof, or if the activity of a polypeptide encoded by a nucleic acid molecule comprising the nucleic acid SEQ ID NO. 40383, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 40383 or polypeptide SEQ ID NO. 40384, respectively, or a homolog or a fragment thereof, is increased or generated, or if the activity 2,3-dihydroxy-2,3-dihydrophenylpropionatedehydrogenase is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 121 to 441-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a 2,3-dihydroxy-2,3-dihydrophenylpropionatedehydrogenase, or if the activity of the polypeptide B2541, preferably represented by SEQ ID NO. 40384, or a homolog or fragment thereof, or if the activity of a polypeptide encoded by a nucleic acid molecule comprising the nucleic acid SEQ ID NO. 40383, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 40383 or polypeptide SEQ ID NO. 40384, respectively, or a homolog or a fragment thereof, is increased or generated, or if the activity 2,3-dihydroxy-2,3-dihydrophenylpropionatedehydrogenase is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 40 to 107-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a b2548-protein, or if the activity of the polypeptide B2548, preferably represented by SEQ ID NO. 40638, or a homolog or fragment thereof, or if the activity of a polypeptide encoded by a nucleic acid molecule comprising the nucleic acid SEQ ID NO. 40637, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 40637 or polypeptide SEQ ID NO. 40638, respectively, or a homolog or a fragment thereof, is increased or generated, or if the activity b2548-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 36 to 62-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a b2613-protein, or if the activity of the polypeptide B2613, preferably represented by SEQ ID NO. 40666, or a homolog or fragment thereof, or if the activity of a polypeptide encoded by a nucleic acid molecule comprising the nucleic acid SEQ ID NO. 40665, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 40665 or polypeptide SEQ ID NO. 40666, respectively, or a homolog or a fragment thereof, is increased or generated, or if the activity b2613-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 38 to 52-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a b2613-protein, or if the activity of the polypeptide B2613, preferably represented by SEQ ID NO. 40666, or a homolog or fragment thereof, or if the activity of a polypeptide encoded by a nucleic acid molecule comprising the nucleic acid SEQ ID NO. 40665, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 40665 or polypeptide SEQ ID NO. 40666, respectively, or a homolog or a fragment thereof, is increased or generated, or if the activity b2613-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 33 to 191-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a transcriptional regulator, or if the activity of the polypeptide B2634, preferably represented by SEQ ID NO. 40727, or a homolog or fragment thereof, or if the activity of a polypeptide encoded by a nucleic acid molecule comprising the nucleic acid SEQ ID NO. 40726, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 40726 or polypeptide SEQ ID NO. 40727, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 56 to 220-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a transcriptional regulator, or if the activity of the polypeptide B2634, preferably represented by SEQ ID NO. 40727, or a homolog or fragment thereof, or if the activity of a polypeptide encoded by a nucleic acid molecule comprising the nucleic acid SEQ ID NO. 40726, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 40726 or polypeptide SEQ ID NO. 40727, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 92 to 1167-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a transcriptional regulator, or if the activity of the polypeptide B2634, preferably represented by SEQ ID NO. 40727, or a homolog or fragment thereof, or if the activity of a polypeptide encoded by a nucleic acid molecule comprising the nucleic acid SEQ ID NO. 40726, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 40726 or polypeptide SEQ ID NO. 40727, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 54 to 1631-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a b2673-protein, or if the activity of the polypeptide B2673, preferably represented by SEQ ID NO. 9245, or a homolog or fragment thereof, or if the activity of a polypeptide encoded by a nucleic acid molecule comprising the nucleic acid SEQ ID NO. 9244, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 9244 or polypeptide SEQ ID NO. 9245, respectively, or a homolog or a fragment thereof, is increased or generated, or if the activity b2673-protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 102 to 157-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a b2673-protein, or if the activity of the polypeptide B2673, preferably represented by SEQ ID NO. 9245, or a homolog or fragment thereof, or if the activity of a polypeptide encoded by a nucleic acid molecule comprising the nucleic acid SEQ ID NO. 9244, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 9244 or polypeptide SEQ ID NO. 9245, respectively, or a homolog or a fragment thereof, is increased or generated, or if the activity b2673-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 126 to 263-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a transcriptional regulator, or if the activity of the polypeptide B2714, preferably represented by SEQ ID NO. 40742, or a homolog or fragment thereof, or if the activity of a polypeptide encoded by a nucleic acid molecule comprising the nucleic acid SEQ ID NO. 40741, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 40741 or polypeptide SEQ ID NO. 40742, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 37 to 46-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a transcriptional regulator, or if the activity of the polypeptide B2714, preferably represented by SEQ ID NO. 40742, or a homolog or fragment thereof, or if the activity of a polypeptide encoded by a nucleic acid molecule comprising the nucleic acid SEQ ID NO. 40741, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 40741 or polypeptide SEQ ID NO. 40742, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 50 to 67-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a b2812-protein, or if the activity of the polypeptide B2812, preferably represented by SEQ ID NO. 40796, or a homolog or fragment thereof, or if the activity of a polypeptide encoded by a nucleic acid molecule comprising the nucleic acid SEQ ID NO. 40795, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 40795 or polypeptide SEQ ID NO. 40796, respectively, or a homolog or a fragment thereof, is increased or generated, or if the activity b2812-protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 37 to 130-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a b2812-protein, or if the activity of the polypeptide B2812, preferably represented by SEQ ID NO. 40796, or a homolog or fragment thereof, or if the activity of a polypeptide encoded by a nucleic acid molecule comprising the nucleic acid SEQ ID NO. 40795, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 40795 or polypeptide SEQ ID NO. 40796, respectively, or a homolog or a fragment thereof, is increased or generated, or if the activity b2812-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 32 to 50-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a b2812-protein, or if the activity of the polypeptide B2812, preferably represented by SEQ ID NO. 40796, or a homolog or fragment thereof, or if the activity of a polypeptide encoded by a nucleic acid molecule comprising the nucleic acid SEQ ID NO. 40795, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 40795 or polypeptide SEQ ID NO. 40796, respectively, or a homolog or a fragment thereof, is increased or generated, or if the activity b2812-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 43 to 104-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a b2846-protein, or if the activity of the polypeptide B2846, preferably represented by SEQ ID NO. 40985, or a homolog or fragment thereof, or if the activity of a polypeptide encoded by a nucleic acid molecule comprising the nucleic acid SEQ ID NO. 40984, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 40984 or polypeptide SEQ ID NO. 40985, respectively, or a homolog or a fragment thereof, is increased or generated, or if the activity b2846-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 50 to 63-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a b2909-protein, or if the activity of the polypeptide B2909, preferably represented by SEQ ID NO. 41007, or a homolog or fragment thereof, or if the activity of a polypeptide encoded by a nucleic acid molecule comprising the nucleic acid SEQ ID NO. 41006, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 41006 or polypeptide SEQ ID NO. 41007, respectively, or a homolog or a fragment thereof, is increased or generated, or if the activity b2909-protein is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 69 to 445-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a b2909-protein, or if the activity of the polypeptide B2909, preferably represented by SEQ ID NO. 41007, or a homolog or fragment thereof, or if the activity of a polypeptide encoded by a nucleic acid molecule comprising the nucleic acid SEQ ID NO. 41006, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 41006 or polypeptide SEQ ID NO. 41007, respectively, or a homolog or a fragment thereof, is increased or generated, or if the activity b2909-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 39 to 248-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a ribosephosphate isomerase, constitutive, or if the activity of the polypeptide B2914, preferably represented by SEQ ID NO. 41074, or a homolog or fragment thereof, or if the activity of a polypeptide encoded by a nucleic acid molecule comprising the nucleic acid SEQ ID NO. 41073, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 41073 or polypeptide SEQ ID NO. 41074, respectively, or a homolog or a fragment thereof, is increased or generated, or if the activity ribosephosphate isomerase, constitutive is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 44 to 95 percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a arginine exporter protein, or if the activity of the polypeptide B2923, preferably represented by SEQ ID NO. 9334, or a homolog or fragment thereof, or if the activity of a polypeptide encoded by a nucleic acid molecule comprising the nucleic acid SEQ ID NO. 9333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 9333 or polypeptide SEQ ID NO. 9334, respectively, or a homolog or a fragment thereof, is increased or generated, or if the activity arginine exporter protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 41 to 77-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a b2936-protein, or if the activity of the polypeptide B2936, preferably represented by SEQ ID NO. 41443, or a homolog or fragment thereof, or if the activity of a polypeptide encoded by a nucleic acid molecule comprising the nucleic acid SEQ ID NO. 41442, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 41442 or polypeptide SEQ ID NO. 41443, respectively, or a homolog or a fragment thereof, is increased or generated, or if the activity b2936-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 50 to 178-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a L-asparaginase, or if the activity of the polypeptide B2957, preferably represented by SEQ ID NO. 41500, or a homolog or fragment thereof, or if the activity of a polypeptide encoded by a nucleic acid molecule comprising the nucleic acid SEQ ID NO. 41499, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 41499 or polypeptide SEQ ID NO. 41500, respectively, or a homolog or a fragment thereof, is increased or generated, or if the activity L-asparaginase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 34 to 262-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a murein transglycosylase, or if the activity of the polypeptide B2963, preferably represented by SEQ ID NO. 41733, or a homolog or fragment thereof, or if the activity of a polypeptide encoded by a nucleic acid molecule comprising the nucleic acid SEQ ID NO. 41732, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 41732 or polypeptide SEQ ID NO. 41733, respectively, or a homolog or a fragment thereof, is increased or generated, or if the activity murein transglycosylase is increased or generated in a non-human organism, like a microorganism or a plant cell, plant or part thereof, especially with plastidic localization, whereby the respective line discloses in column 7 proline. For example, an increase of the proline of at least 1 percent, particularly in a range of 53 to 57-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a b2999-protein, or if the activity of the polypeptide B2999, preferably represented by SEQ ID NO. 41798, or a homolog or fragment thereof, or if the activity of a polypeptide encoded by a nucleic acid molecule comprising the nucleic acid SEQ ID NO. 41797, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 41797 or polypeptide SEQ ID NO. 41798, respectively, or a homolog or a fragment thereof, is increased or generated, or if the activity b2999-protein is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 52 to 278-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a glycoprotease, or if the activity of the polypeptide B3064, preferably represented by SEQ ID NO. 42047, or a homolog or fragment thereof, or if the activity of a polypeptide encoded by a nucleic acid molecule comprising the nucleic acid SEQ ID NO. 42046, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 42046 or polypeptide SEQ ID NO. 42047, respectively, or a homolog or a fragment thereof, is increased or generated, or if the activity glycoprotease is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 57 to 255-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a b3121-protein, or if the activity of the polypeptide B3121, preferably represented by SEQ ID NO. 42472, or a homolog or fragment thereof, or if the activity of a polypeptide encoded by a nucleic acid molecule comprising the nucleic acid SEQ ID NO. 42471, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 42471 or polypeptide SEQ ID NO. 42472, respectively, or a homolog or a fragment thereof, is increased or generated, or if the activity b3121-protein is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 63 to 201-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a b3121-protein, or if the activity of the polypeptide B3121, preferably represented by SEQ ID NO. 42472, or a homolog or fragment thereof, or if the activity of a polypeptide encoded by a nucleic acid molecule comprising the nucleic acid SEQ ID NO. 42471, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 42471 or polypeptide SEQ ID NO. 42472, respectively, or a homolog or a fragment thereof, is increased or generated, or if the activity b3121-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 33 to 104-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a b3151-protein, or if the activity of the polypeptide B3151, preferably represented by SEQ ID NO. 42478, or a homolog or fragment thereof, or if the activity of a polypeptide encoded by a nucleic acid molecule comprising the nucleic acid SEQ ID NO. 42477, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 42477 or polypeptide SEQ ID NO. 42478, respectively, or a homolog or a fragment thereof, is increased or generated, or if the activity b3151-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 45 to 689-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a acetyl CoA carboxylase, or if the activity of the polypeptide B3256, preferably represented by SEQ ID NO. 9493, or a homolog or fragment thereof, or if the activity of a polypeptide encoded by a nucleic acid molecule comprising the nucleic acid SEQ ID NO. 9492, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 9492 or polypeptide SEQ ID NO. 9493, respectively, or a homolog or a fragment thereof, is increased or generated, or if the activity acetyl CoA carboxylase is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 100 to 2020-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a acetyl CoA carboxylase, or if the activity of the polypeptide B3256, preferably represented by SEQ ID NO. 9493, or a homolog or fragment thereof, or if the activity of a polypeptide encoded by a nucleic acid molecule comprising the nucleic acid SEQ ID NO. 9492, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 9492 or polypeptide SEQ ID NO. 9493, respectively, or a homolog or a fragment thereof, is increased or generated, or if the activity acetyl CoA carboxylase is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 57 to 194-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a methyltransferase, or if the activity of the polypeptide B3262, preferably represented by SEQ ID NO. 42503, or a homolog or fragment thereof, or if the activity of a polypeptide encoded by a nucleic acid molecule comprising the nucleic acid SEQ ID NO. 42502, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 42502 or polypeptide SEQ ID NO. 42503, respectively, or a homolog or a fragment thereof, is increased or generated, or if the activity methyltransferase is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 50 to 273-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a methyltransferase, or if the activity of the polypeptide B3262, preferably represented by SEQ ID NO. 42503, or a homolog or fragment thereof, or if the activity of a polypeptide encoded by a nucleic acid molecule comprising the nucleic acid SEQ ID NO. 42502, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 42502 or polypeptide SEQ ID NO. 42503, respectively, or a homolog or a fragment thereof, is increased or generated, or if the activity methyltransferase is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 79 to 126-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a methyltransferase, or if the activity of the polypeptide B3262, preferably represented by SEQ ID NO. 42503, or a homolog or fragment thereof, or if the activity of a polypeptide encoded by a nucleic acid molecule comprising the nucleic acid SEQ ID NO. 42502, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 42502 or polypeptide SEQ ID NO. 42503, respectively, or a homolog or a fragment thereof, is increased or generated, or if the activity methyltransferase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 27 to 113-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a methyltransferase, or if the activity of the polypeptide B3262, preferably represented by SEQ ID NO. 42503, or a homolog or fragment thereof, or if the activity of a polypeptide encoded by a nucleic acid molecule comprising the nucleic acid SEQ ID NO. 42502, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 42502 or polypeptide SEQ ID NO. 42503, respectively, or a homolog or a fragment thereof, is increased or generated, or if the activity methyltransferase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 78 to 302-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a b3346-protein, or if the activity of the polypeptide B3346, preferably represented by SEQ ID NO. 10105, or a homolog or fragment thereof, or if the activity of a polypeptide encoded by a nucleic acid molecule comprising the nucleic acid SEQ ID NO. 10104, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 10104 or polypeptide SEQ ID NO. 10105, respectively, or a homolog or a fragment thereof, is increased or generated, or if the activity b3346-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 48 to 193-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a b3410-protein, or if the activity of the polypeptide B3410, preferably represented by SEQ ID NO. 42560, or a homolog or fragment thereof, or if the activity of a polypeptide encoded by a nucleic acid molecule comprising the nucleic acid SEQ ID NO. 42559, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 42559 or polypeptide SEQ ID NO. 42560, respectively, or a homolog or a fragment thereof, is increased or generated, or if the activity b3410-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 40 to 86-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a B3427-protein, or if the activity of the polypeptide B3427, preferably represented by SEQ ID NO. 42580, or a homolog or fragment thereof, or if the activity of a polypeptide encoded by a nucleic acid molecule comprising the nucleic acid SEQ ID NO. 42579, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 42579 or polypeptide SEQ ID NO. 42580, respectively, or a homolog or a fragment thereof, is increased or generated, or if the activity B3427-protein is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 66 to 148-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a b3509-protein, or if the activity of the polypeptide B3509, preferably represented by SEQ ID NO. 42593, or a homolog or fragment thereof, or if the activity of a polypeptide encoded by a nucleic acid molecule comprising the nucleic acid SEQ ID NO. 42592, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 42592 or polypeptide SEQ ID NO. 42593, respectively, or a homolog or a fragment thereof, is increased or generated, or if the activity b3509-protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 47 to 65-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a valine-pyruvate transaminase, or if the activity of the polypeptide B3572, preferably represented by SEQ ID NO. 10173, or a homolog or fragment thereof, or if the activity of a polypeptide encoded by a nucleic acid molecule comprising the nucleic acid SEQ ID NO. 10172, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 10172 or polypeptide SEQ ID NO. 10173, respectively, or a homolog or a fragment thereof, is increased or generated, or if the activity valine-pyruvate transaminase is increased or generated in a non-human organism, like a microorganism or a plant cell, plant or part thereof, especially with plastidic localization, whereby the respective line discloses in column 7 proline. For example, an increase of the proline of at least 1 percent, particularly in a range of 55 to 376-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a threonine dehydrogenase, or if the activity of the polypeptide B3616, preferably represented by SEQ ID NO. 42601, or a homolog or fragment thereof, or if the activity of a polypeptide encoded by a nucleic acid molecule comprising the nucleic acid SEQ ID NO. 42600, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 42600 or polypeptide SEQ ID NO. 42601, respectively, or a homolog or a fragment thereof, is increased or generated, or if the activity threonine dehydrogenase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 45 to 225-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a threonine dehydrogenase, or if the activity of the polypeptide B3616, preferably represented by SEQ ID NO. 42601, or a homolog or fragment thereof, or if the activity of a polypeptide encoded by a nucleic acid molecule comprising the nucleic acid SEQ ID NO. 42600, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 42600 or polypeptide SEQ ID NO. 42601, respectively, or a homolog or a fragment thereof, is increased or generated, or if the activity threonine dehydrogenase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 49 to 107-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a phosphopantetheine adenylyltransferase, or if the activity of the polypeptide B3634, preferably represented by SEQ ID NO. 42932, or a homolog or fragment thereof, or if the activity of a polypeptide encoded by a nucleic acid molecule comprising the nucleic acid SEQ ID NO. 42931, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 42931 or polypeptide SEQ ID NO. 42932, respectively, or a homolog or a fragment thereof, is increased or generated, or if the activity phosphopantetheine adenylyltransferase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 34 to 162-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a ATP synthase subunit beta, or if the activity of the polypeptide B3732, preferably represented by SEQ ID NO. 43249, or a homolog or fragment thereof, or if the activity of a polypeptide encoded by a nucleic acid molecule comprising the nucleic acid SEQ ID NO. 43248, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 43248 or polypeptide SEQ ID NO. 43249, respectively, or a homolog or a fragment thereof, is increased or generated, or if the activity ATP synthase subunit beta is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 30 to 36-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a enterobacterial common antigen polymerase, or if the activity of the polypeptide B3793, preferably represented by SEQ ID NO. 43801, or a homolog or fragment thereof, or if the activity of a polypeptide encoded by a nucleic acid molecule comprising the nucleic acid SEQ ID NO. 43800, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 43800 or polypeptide SEQ ID NO. 43801, respectively, or a homolog or a fragment thereof, is increased or generated, or if the activity enterobacterial common antigen polymerase is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 58 to 229-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a enterobacterial common antigen polymerase, or if the activity of the polypeptide B3793, preferably represented by SEQ ID NO. 43801, or a homolog or fragment thereof, or if the activity of a polypeptide encoded by a nucleic acid molecule comprising the nucleic acid SEQ ID NO. 43800, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 43800 or polypeptide SEQ ID NO. 43801, respectively, or a homolog or a fragment thereof, is increased or generated, or if the activity enterobacterial common antigen polymerase is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 78 to 212 percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a DNA helicase II, or if the activity of the polypeptide B3813, preferably represented by SEQ ID NO. 43840, or a homolog or fragment thereof, or if the activity of a polypeptide encoded by a nucleic acid molecule comprising the nucleic acid SEQ ID NO. 43839, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 43839 or polypeptide SEQ ID NO. 43840, respectively, or a homolog or a fragment thereof, is increased or generated, or if the activity DNA helicase II is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 104 to 159-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a b3814-protein, or if the activity of the polypeptide B3814, preferably represented by SEQ ID NO. 44197, or a homolog or fragment thereof, or if the activity of a polypeptide encoded by a nucleic acid molecule comprising the nucleic acid SEQ ID NO. 44196, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 44196 or polypeptide SEQ ID NO. 44197, respectively, or a homolog or a fragment thereof, is increased or generated, or if the activity b3814-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 54 to 94-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a b3817-protein, or if the activity of the polypeptide B3817, preferably represented by SEQ ID NO. 10709, or a homolog or fragment thereof, or if the activity of a polypeptide encoded by a nucleic acid molecule comprising the nucleic acid SEQ ID NO. 10708, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 10708 or polypeptide SEQ ID NO. 10709, respectively, or a homolog or a fragment thereof, is increased or generated, or if the activity b3817-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 607 to 890-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a glycerol dehydrogenase, or if the activity of the polypeptide B3945, preferably represented by SEQ ID NO. 44224, or a homolog or fragment thereof, or if the activity of a polypeptide encoded by a nucleic acid molecule comprising the nucleic acid SEQ ID NO. 44223, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 44223 or polypeptide SEQ ID NO. 44224, respectively, or a homolog or a fragment thereof, is increased or generated, or if the activity glycerol dehydrogenase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 68 to 125-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a b3989-protein, or if the activity of the polypeptide B3989, preferably represented by SEQ ID NO. 44373, or a homolog or fragment thereof, or if the activity of a polypeptide encoded by a nucleic acid molecule comprising the nucleic acid SEQ ID NO. 44372, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 44372 or polypeptide SEQ ID NO. 44373, respectively, or a homolog or a fragment thereof, is increased or generated, or if the activity b3989-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 28 to 90-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a acetyltransferase, or if the activity of the polypeptide B4012, preferably represented by SEQ ID NO. 44379, or a homolog or fragment thereof, or if the activity of a polypeptide encoded by a nucleic acid molecule comprising the nucleic acid SEQ ID NO. 44378, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 44378 or polypeptide SEQ ID NO. 44379, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 39 to 76-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a b4029-protein, or if the activity of the polypeptide B4029, preferably represented by SEQ ID NO. 10741, or a homolog or fragment thereof, or if the activity of a polypeptide encoded by a nucleic acid molecule comprising the nucleic acid SEQ ID NO. 10740, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 10740 or polypeptide SEQ ID NO. 10741, respectively, or a homolog or a fragment thereof, is increased or generated, or if the activity b4029-protein is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 58 to 478-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a b4029-protein, or if the activity of the polypeptide B4029, preferably represented by SEQ ID NO. 10741, or a homolog or fragment thereof, or if the activity of a polypeptide encoded by a nucleic acid molecule comprising the nucleic acid SEQ ID NO. 10740, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 10740 or polypeptide SEQ ID NO. 10741, respectively, or a homolog or a fragment thereof, is increased or generated, or if the activity b4029-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 52 to 2052-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a b4029-protein, or if the activity of the polypeptide B4029, preferably represented by SEQ ID NO. 10741, or a homolog or fragment thereof, or if the activity of a polypeptide encoded by a nucleic acid molecule comprising the nucleic acid SEQ ID NO. 10740, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 10740 or polypeptide SEQ ID NO. 10741, respectively, or a homolog or a fragment thereof, is increased or generated, or if the activity b4029-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 32 to 836-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a cation/acetate symporter, or if the activity of the polypeptide B4067, preferably represented by SEQ ID NO. 44467, or a homolog or fragment thereof, or if the activity of a polypeptide encoded by a nucleic acid molecule comprising the nucleic acid SEQ ID NO. 44466, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 44466 or polypeptide SEQ ID NO. 44467, respectively, or a homolog or a fragment thereof, is increased or generated, or if the activity cation/acetate symporter is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 46 to 154-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a b4121-protein, or if the activity of the polypeptide B4121, preferably represented by SEQ ID NO. 44610, or a homolog or fragment thereof, or if the activity of a polypeptide encoded by a nucleic acid molecule comprising the nucleic acid SEQ ID NO. 44609, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 44609 or polypeptide SEQ ID NO. 44610, respectively, or a homolog or a fragment thereof, is increased or generated, or if the activity b4121-protein is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 46 to 442-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a b4121-protein, or if the activity of the polypeptide B4121, preferably represented by SEQ ID NO. 44610, or a homolog or fragment thereof, or if the activity of a polypeptide encoded by a nucleic acid molecule comprising the nucleic acid SEQ ID NO. 44609, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 44609 or polypeptide SEQ ID NO. 44610, respectively, or a homolog or a fragment thereof, is increased or generated, or if the activity b4121-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 33 to 151-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a lysyl-tRNA synthetase, or if the activity of the polypeptide B4129, preferably represented by SEQ ID NO. 44663, or a homolog or fragment thereof, or if the activity of a polypeptide encoded by a nucleic acid molecule comprising the nucleic acid SEQ ID NO. 44662, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 44662 or polypeptide SEQ ID NO. 44663, respectively, or a homolog or a fragment thereof, is increased or generated, or if the activity lysyl-tRNA synthetase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 126 to 168-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a aspartase, or if the activity of the polypeptide B4139, preferably represented by SEQ ID NO. 45023, or a homolog or fragment thereof, or if the activity of a polypeptide encoded by a nucleic acid molecule comprising the nucleic acid SEQ ID NO. 45022, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 45022 or polypeptide SEQ ID NO. 45023, respectively, or a homolog or a fragment thereof, is increased or generated, or if the activity aspartase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 196 to 629-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a acetyltransferase, or if the activity of the polypeptide B4256, preferably represented by SEQ ID NO. 45322, or a homolog or fragment thereof, or if the activity of a polypeptide encoded by a nucleic acid molecule comprising the nucleic acid SEQ ID NO. 45321, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 45321 or polypeptide SEQ ID NO. 45322, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 93 to 838-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a acetyltransferase, or if the activity of the polypeptide B4256, preferably represented by SEQ ID NO. 45322, or a homolog or fragment thereof, or if the activity of a polypeptide encoded by a nucleic acid molecule comprising the nucleic acid SEQ ID NO. 45321, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 45321 or polypeptide SEQ ID NO. 45322, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 43 to 153-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a acetyltransferase, or if the activity of the polypeptide B4256, preferably represented by SEQ ID NO. 45322, or a homolog or fragment thereof, or if the activity of a polypeptide encoded by a nucleic acid molecule comprising the nucleic acid SEQ ID NO. 45321, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 45321 or polypeptide SEQ ID NO. 45322, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 39 to 843-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a gluconate transport system permease 3, or if the activity of the polypeptide B4321, preferably represented by SEQ ID NO. 45395, or a homolog or fragment thereof, or if the activity of a polypeptide encoded by a nucleic acid molecule comprising the nucleic acid SEQ ID NO. 45394, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 45394 or polypeptide SEQ ID NO. 45395, respectively, or a homolog or a fragment thereof, is increased or generated, or if the activity gluconate transport system permease 3 is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 75 to 181 percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a purinenucleoside phosphorylase, or if the activity of the polypeptide B4384, preferably represented by SEQ ID NO. 45557, or a homolog or fragment thereof, or if the activity of a polypeptide encoded by a nucleic acid molecule comprising the nucleic acid SEQ ID NO. 45556, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 45556 or polypeptide SEQ ID NO. 45557, respectively, or a homolog or a fragment thereof, is increased or generated, or if the activity purine-nucleoside phosphorylase is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 59 to 124-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a purine-nucleoside phosphorylase, or if the activity of the polypeptide B4384, preferably represented by SEQ ID NO. 45557, or a homolog or fragment thereof, or if the activity of a polypeptide encoded by a nucleic acid molecule comprising the nucleic acid SEQ ID NO. 45556, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 45556 or polypeptide SEQ ID NO. 45557, respectively, or a homolog or a fragment thereof, is increased or generated, or if the activity purine-nucleoside phosphorylase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 107 to 506-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a thioredoxin, or if the activity of the polypeptide C_pp004096192r, preferably represented by SEQ ID NO. 45758, or a homolog or fragment thereof, or if the activity of a polypeptide encoded by a nucleic acid molecule comprising the nucleic acid SEQ ID NO. 45757, preferably the coding region thereof, or a homolog or fragment thereof, e.g. derived from Physcomitrella patens, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 45757 or polypeptide SEQ ID NO. 45758, respectively, or a homolog or a fragment thereof, is increased or generated, or if the activity thioredoxin is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 61 to 306-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a GM02LC11114-protein, or if the activity of the polypeptide GM02LC11114, preferably represented by SEQ ID NO. 45796, or a homolog or fragment thereof, or if the activity of a polypeptide encoded by a nucleic acid molecule comprising the nucleic acid SEQ ID NO. 45795, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 45795 or polypeptide SEQ ID NO. 45796, respectively, or a homolog or a fragment thereof, is increased or generated, or if the activity GM02LC11114-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 40 to 127-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a glutaredoxin, or if the activity of the polypeptide GM02LC12622, preferably represented by SEQ ID NO. 10812, or a homolog or fragment thereof, or if the activity of a polypeptide encoded by a nucleic acid molecule comprising the nucleic acid SEQ ID NO. 10811, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 10811 or polypeptide SEQ ID NO. 10812, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 68 to 261-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a histone H2A, or if the activity of the polypeptide GM02LC15313, preferably represented by SEQ ID NO. 45898, or a homolog or fragment thereof, or if the activity of a polypeptide encoded by a nucleic acid molecule comprising the nucleic acid SEQ ID NO. 45897, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 45897 or polypeptide SEQ ID NO. 45898, respectively, or a homolog or a fragment thereof, is increased or generated, or if the activity histone H2A is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 63 to 488-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a histone H2A, or if the activity of the polypeptide GM02LC15313, preferably represented by SEQ ID NO. 45898, or a homolog or fragment thereof, or if the activity of a polypeptide encoded by a nucleic acid molecule comprising the nucleic acid SEQ ID NO. 45897, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 45897 or polypeptide SEQ ID NO. 45898, respectively, or a homolog or a fragment thereof, is increased or generated, or if the activity histone H2A is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 37 to 144-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a GM02LC17485-protein, or if the activity of the polypeptide GM02LC17485, preferably represented by SEQ ID NO. 46406, or a homolog or fragment thereof, or if the activity of a polypeptide encoded by a nucleic acid molecule comprising the nucleic acid SEQ ID NO. 46405, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 46405 or polypeptide SEQ ID NO. 46406, respectively, or a homolog or a fragment thereof, is increased or generated, or if the activity GM02LC17485-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 45 to 157-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a RNA binding protein, or if the activity of the polypeptide GM02LC17556, preferably represented by SEQ ID NO. 46516, or a homolog or fragment thereof, or if the activity of a polypeptide encoded by a nucleic acid molecule comprising the nucleic acid SEQ ID NO. 46515, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 46515 or polypeptide SEQ ID NO. 46516, respectively, or a homolog or a fragment thereof, is increased or generated, or if the activity RNA binding protein is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 98 to 362-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a transcription factor, or if the activity of the polypeptide GM02LC19289, preferably represented by SEQ ID NO. 46752, or a homolog or fragment thereof, or if the activity of a polypeptide encoded by a nucleic acid molecule comprising the nucleic acid SEQ ID NO. 46751, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 46751 or polypeptide SEQ ID NO. 46752, respectively, or a homolog or a fragment thereof, is increased or generated, or if the activity transcription factor is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 36 to 231-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a cyclin D, or if the activity of the polypeptide GM02LC44512, preferably represented by SEQ ID NO. 46851, or a homolog or fragment thereof, or if the activity of a polypeptide encoded by a nucleic acid molecule comprising the nucleic acid SEQ ID NO. 46850, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 46850 or polypeptide SEQ ID NO. 46851, respectively, or a homolog or a fragment thereof, is increased or generated, or if the activity cyclin D is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 72 to 410-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a GM02LC46-protein, or if the activity of the polypeptide GM02LC46, preferably represented by SEQ ID NO. 47027, or a homolog or fragment thereof, or if the activity of a polypeptide encoded by a nucleic acid molecule comprising the nucleic acid SEQ ID NO. 47026, preferably the coding region thereof, or a homolog or fragment thereof, e.g. derived from GLYCINE MAX, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 47026 or polypeptide SEQ ID NO. 47027, respectively, or a homolog or a fragment thereof, is increased or generated, or if the activity GM02LC46-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 59 to 113-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a GM02LC5744-protein, or if the activity of the polypeptide GM02LC5744, preferably represented by SEQ ID NO. 47077, or a homolog or fragment thereof, or if the activity of a polypeptide encoded by a nucleic acid molecule comprising the nucleic acid SEQ ID NO. 47076, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 47076 or polypeptide SEQ ID NO. 47077, respectively, or a homolog or a fragment thereof, is increased or generated, or if the activity GM02LC5744-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 42 to 73-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a transcription factor, or if the activity of the polypeptide GM02LC6021, preferably represented by SEQ ID NO. 47106, or a homolog or fragment thereof, or if the activity of a polypeptide encoded by a nucleic acid molecule comprising the nucleic acid SEQ ID NO. 47105, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 47105 or polypeptide SEQ ID NO. 47106, respectively, or a homolog or a fragment thereof, is increased or generated, or if the activity transcription factor is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 37 to 66-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a glutaredoxin, or if the activity of the polypeptide NZ_AAAU02000016.150, preferably represented by SEQ ID NO. 47160, or a homolog or fragment thereof, or if the activity of a polypeptide encoded by a nucleic acid molecule comprising the nucleic acid SEQ ID NO. 47159, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 47159 or polypeptide SEQ ID NO. 47160, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 37 to 58-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a s_pp015018333r-protein, or if the activity of the polypeptide S_pp015018333r, preferably represented by SEQ ID NO. 59852, or a homolog or fragment thereof, or if the activity of a polypeptide encoded by a nucleic acid molecule comprising the nucleic acid SEQ ID NO. 59851, preferably the coding region thereof, or a homolog or fragment thereof, e.g. derived from Physcomitrella patens, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 59851 or polypeptide SEQ ID NO. 59852, respectively, or a homolog or a fragment thereof, is increased or generated, or if the activity s_pp015018333r-protein is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 106 to 460-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a s_pp015018333r-protein, or if the activity of the polypeptide S_pp015018333r, preferably represented by SEQ ID NO. 59852, or a homolog or fragment thereof, or if the activity of a polypeptide encoded by a nucleic acid molecule comprising the nucleic acid SEQ ID NO. 59851, preferably the coding region thereof, or a homolog or fragment thereof, e.g. derived from Physcomitrella patens, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 59851 or polypeptide SEQ ID NO. 59852, respectively, or a homolog or a fragment thereof, is increased or generated, or if the activity s_pp015018333r-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 44 to 187-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a sll0064-protein, or if the activity of the polypeptide Sll0064, preferably represented by SEQ ID NO. 47527, or a homolog or fragment thereof, or if the activity of a polypeptide encoded by a nucleic acid molecule comprising the nucleic acid SEQ ID NO. 47526, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 47526 or polypeptide SEQ ID NO. 47527, respectively, or a homolog or a fragment thereof, is increased or generated, or if the activity sll0064-protein is increased or generated in a non-human organism, like a microorganism or a plant cell, plant or part thereof, especially with mitochondrial localization, whereby the respective line discloses in column 7 proline. For example, an increase of the proline of at least 1 percent, particularly in a range of 62 to 111-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a N-acetyl-gamma-glutamyl-phosphate reductase, or if the activity of the polypeptide Sll0080, preferably represented by SEQ ID NO. 47567, or a homolog or fragment thereof, or if the activity of a polypeptide encoded by a nucleic acid molecule comprising the nucleic acid SEQ ID NO. 47566, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 47566 or polypeptide SEQ ID NO. 47567, respectively, or a homolog or a fragment thereof, is increased or generated, or if the activity N-acetyl-gamma-glutamyl-phosphate reductase is increased or generated in a non-human organism, like a microorganism or a plant cell, plant or part thereof, especially with plastidic localization, whereby the respective line discloses in column 7 glutamate. For example, an increase of the glutamate of at least 1 percent, particularly in a range of 51 to 61-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a heat shock protein, or if the activity of the polypeptide Sll0170, preferably represented by SEQ ID NO. 48139, or a homolog or fragment thereof, or if the activity of a polypeptide encoded by a nucleic acid molecule comprising the nucleic acid SEQ ID NO. 48138, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 48138 or polypeptide SEQ ID NO. 48139, respectively, or a homolog or a fragment thereof, is increased or generated, or if the activity heat shock protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 39 to 234-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a flavodoxin, or if the activity of the polypeptide Sll0248, preferably represented by SEQ ID NO. 49144, or a homolog or fragment thereof, or if the activity of a polypeptide encoded by a nucleic acid molecule comprising the nucleic acid SEQ ID NO. 49143, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 49143 or polypeptide SEQ ID NO. 49144, respectively, or a homolog or a fragment thereof, is increased or generated, or if the activity flavodoxin is increased or generated in a non-human organism, like a microorganism or a plant cell, plant or part thereof, especially with plastidic localization, whereby the respective line discloses in column 7 arginine. For example, an increase of the arginine of at least 1 percent, particularly in a range of 49 to 438-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a phosphopantothenoylcysteinesynthetase/decarboxylase, or if the activity of the polypeptide Sll0250, preferably represented by SEQ ID NO. 49343, or a homolog or fragment thereof, or if the activity of a polypeptide encoded by a nucleic acid molecule comprising the nucleic acid SEQ ID NO. 49342, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 49342 or polypeptide SEQ ID NO. 49343, respectively, or a homolog or a fragment thereof, is increased or generated, or if the activity phosphopantothenoylcysteinesynthetase/decarboxylase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 139 to 160-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a sll0254-protein, or if the activity of the polypeptide Sll0254, preferably represented by SEQ ID NO. 49801, or a homolog or fragment thereof, or if the activity of a polypeptide encoded by a nucleic acid molecule comprising the nucleic acid SEQ ID NO. 49800, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 49800 or polypeptide SEQ ID NO. 49801, respectively, or a homolog or a fragment thereof, is increased or generated, or if the activity sll0254-protein is increased or generated in a non-human organism, like a microorganism or a plant cell, plant or part thereof, especially with mitochondrial localization, whereby the respective line discloses in column 7 glutamine. For example, an increase of the glutamine of at least 1 percent, particularly in a range of 49 to 391-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a sll0254-protein, or if the activity of the polypeptide Sll0254, preferably represented by SEQ ID NO. 49801, or a homolog or fragment thereof, or if the activity of a polypeptide encoded by a nucleic acid molecule comprising the nucleic acid SEQ ID NO. 49800, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 49800 or polypeptide SEQ ID NO. 49801, respectively, or a homolog or a fragment thereof, is increased or generated, or if the activity sll0254-protein is increased or generated in a non-human organism, like a microorganism or a plant cell, plant or part thereof, especially with mitochondrial localization, whereby the respective line discloses in column 7 proline. For example, an increase of the proline of at least 1 percent, particularly in a range of 40 to 179-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a polyphosphate kinase, or if the activity of the polypeptide Sll0290, preferably represented by SEQ ID NO. 49829, or a homolog or fragment thereof, or if the activity of a polypeptide encoded by a nucleic acid molecule comprising the nucleic acid SEQ ID NO. 49828, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 49828 or polypeptide SEQ ID NO. 49829, respectively, or a homolog or a fragment thereof, is increased or generated, or if the activity polyphosphate kinase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 34 to 190-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a sll0354-protein, or if the activity of the polypeptide Sll0354, preferably represented by SEQ ID NO. 50071, or a homolog or fragment thereof, or if the activity of a polypeptide encoded by a nucleic acid molecule comprising the nucleic acid SEQ ID NO. 50070, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 50070 or polypeptide SEQ ID NO. 50071, respectively, or a homolog or a fragment thereof, is increased or generated, or if the activity sll0354-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 42 to 129-percent is conferred as compared to a corresponding non-transformed wild type non-human organism. Accordingly, in one embodiment, an increase of proline in a non-human organism, as compared to a corresponding non-transformed wild type non-human organism, is conferred in the process of the invention, if the activity of a polypeptide showing the activity of a urease subunit, or if the activity of the polypeptide Sll0420, preferably represented by SEQ ID NO. 50105, or a homolog or fragment thereof, or if the activity of a polypeptide encoded by a nucleic acid molecule comprising the nucleic acid SEQ ID NO. 50104, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 50104 or polypeptide SEQ ID NO. 50105, respectively, or a homolog or a fragment thereof, is increased or generated, or if the activity urease subunit is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 33 to 45-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a NAD(P)Hquinone oxidoreductase subunit, or if the activity of the polypeptide Sll0521, preferably represented by SEQ ID NO. 50340, or a homolog or fragment thereof, or if the activity of a polypeptide encoded by a nucleic acid molecule comprising the nucleic acid SEQ ID NO. 50339, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 50339 or polypeptide SEQ ID NO. 50340, respectively, or a homolog or a fragment thereof, is increased or generated, or if the activity NAD(P)H-quinone oxidoreductase subunit is increased or generated in a non-human organism, like a microorganism or a plant cell, plant or part thereof, especially with plastidic localization, whereby the respective line discloses in column 7 arginine. For example, an increase of the arginine of at least 1 percent, particularly in a range of 51 to 185 percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a quinolinate synthetase, or if the activity of the polypeptide Sll0622, preferably represented by SEQ ID NO. 50714, or a homolog or fragment thereof, or if the activity of a polypeptide encoded by a nucleic acid molecule comprising the nucleic acid SEQ ID NO. 50713, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 50713 or polypeptide SEQ ID NO. 50714, respectively, or a homolog or a fragment thereof, is increased or generated, or if the activity quinolinate synthetase is increased or generated in a non-human organism, like a microorganism or a plant cell, plant or part thereof, especially with mitochondrial localization, whereby the respective line discloses in column 7 proline. For example, an increase of the proline of at least 1 percent, particularly in a range of 46 to 106-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a permease protein of phosphate ABC transporter, or if the activity of the polypeptide Sll0682, preferably represented by SEQ ID NO. 50951, or a homolog or fragment thereof, or if the activity of a polypeptide encoded by a nucleic acid molecule comprising the nucleic acid SEQ ID NO. 50950, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 50950 or polypeptide SEQ ID NO. 50951, respectively, or a homolog or a fragment thereof, is increased or generated, or if the activity permease protein of phosphate ABC transporter is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 45 to 153-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a permease protein of phosphate ABC transporter, or if the activity of the polypeptide Sll0682, preferably represented by SEQ ID NO. 50951, or a homolog or fragment thereof, or if the activity of a polypeptide encoded by a nucleic acid molecule comprising the nucleic acid SEQ ID NO. 50950, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 50950 or polypeptide SEQ ID NO. 50951, respectively, or a homolog or a fragment thereof, is increased or generated, or if the activity permease protein of phosphate ABC transporter is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 32 to 90 percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a oxireductase, or if the activity of the polypeptide Sll0816, preferably represented by SEQ ID NO. 51199, or a homolog or fragment thereof, or if the activity of a polypeptide encoded by a nucleic acid molecule comprising the nucleic acid SEQ ID NO. 51198, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 51198 or polypeptide SEQ ID NO. 51199, respectively, or a homolog or a fragment thereof, is increased or generated, or if the activity oxireductase is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 61 to 392-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a oxireductase, or if the activity of the polypeptide Sll0816, preferably represented by SEQ ID NO. 51199, or a homolog or fragment thereof, or if the activity of a polypeptide encoded by a nucleic acid molecule comprising the nucleic acid SEQ ID NO. 51198, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 51198 or polypeptide SEQ ID NO. 51199, respectively, or a homolog or a fragment thereof, is increased or generated, or if the activity oxireductase is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 39 to 190-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a oxireductase, or if the activity of the polypeptide Sll0816, preferably represented by SEQ ID NO. 51199, or a homolog or fragment thereof, or if the activity of a polypeptide encoded by a nucleic acid molecule comprising the nucleic acid SEQ ID NO. 51198, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 51198 or polypeptide SEQ ID NO. 51199, respectively, or a homolog or a fragment thereof, is increased or generated, or if the activity oxireductase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 81 to 382-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a malate dehydrogenase, or if the activity of the polypeptide Sll0891, preferably represented by SEQ ID NO. 51269, or a homolog or fragment thereof, or if the activity of a polypeptide encoded by a nucleic acid molecule comprising the nucleic acid SEQ ID NO. 51268, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 51268 or polypeptide SEQ ID NO. 51269, respectively, or a homolog or a fragment thereof, is increased or generated, or if the activity malate dehydrogenase is increased or generated in a non-human organism, like a microorganism or a plant cell, plant or part thereof, especially with plastidic localization, whereby the respective line discloses in column 7 arginine. For example, an increase of the arginine of at least 1 percent, particularly in a range of 47 to 289-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a malate dehydrogenase, or if the activity of the polypeptide Sll0891, preferably represented by SEQ ID NO. 51269, or a homolog or fragment thereof, or if the activity of a polypeptide encoded by a nucleic acid molecule comprising the nucleic acid SEQ ID NO. 51268, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 51268 or polypeptide SEQ ID NO. 51269, respectively, or a homolog or a fragment thereof, is increased or generated, or if the activity malate dehydrogenase is increased or generated in a non-human organism, like a microorganism or a plant cell, plant or part thereof, especially with plastidic localization, whereby the respective line discloses in column 7 glutamine. For example, an increase of the glutamine of at least 1 percent, particularly in a range of 28 to 28-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a malate dehydrogenase, or if the activity of the polypeptide Sll0891, preferably represented by SEQ ID NO. 51269, or a homolog or fragment thereof, or if the activity of a polypeptide encoded by a nucleic acid molecule comprising the nucleic acid SEQ ID NO. 51268, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 51268 or polypeptide SEQ ID NO. 51269, respectively, or a homolog or a fragment thereof, is increased or generated, or if the activity malate dehydrogenase is increased or generated in a non-human organism, like a microorganism or a plant cell, plant or part thereof, especially with plastidic localization, whereby the respective line discloses in column 7 proline. For example, an increase of the proline of at least 1 percent, particularly in a range of 52 to 77-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a phosphoribosylaminoimidazole carboxylase catalytic subunit, or if the activity of the polypeptide Sll0901, preferably represented by SEQ ID NO. 51633, or a homolog or fragment thereof, or if the activity of a polypeptide encoded by a nucleic acid molecule comprising the nucleic acid SEQ ID NO. 51632, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 51632 or polypeptide SEQ ID NO. 51633, respectively, or a homolog or a fragment thereof, is increased or generated, or if the activity phosphoribosylaminoimidazole carboxylase catalytic subunit is increased or generated in a non-human organism, like a microorganism or a plant cell, plant or part thereof, especially with mitochondrial localization, whereby the respective line discloses in column 7 proline. For example, an increase of the proline of at least 1 percent, particularly in a range of 38 to 273-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a 3-deoxy-7-phosphoheptulonate synthase, or if the activity of the polypeptide Sll0934, preferably represented by SEQ ID NO. 52247, or a homolog or fragment thereof, or if the activity of a polypeptide encoded by a nucleic acid molecule comprising the nucleic acid SEQ ID NO. 52246, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 52246 or polypeptide SEQ ID NO. 52247, respectively, or a homolog or a fragment thereof, is increased or generated, or if the activity 3-deoxy-7-phosphoheptulonate synthase is increased or generated in a non-human organism, like a microorganism or a plant cell, plant or part thereof, especially with mitochondrial localization, whereby the respective line discloses in column 7 glutamine. For example, an increase of the glutamine of at least 1 percent, particularly in a range of 72 to 148-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a glycogen synthase, or if the activity of the polypeptide Sll0945, preferably represented by SEQ ID NO. 52365, or a homolog or fragment thereof, or if the activity of a polypeptide encoded by a nucleic acid molecule comprising the nucleic acid SEQ ID NO. 52364, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 52364 or polypeptide SEQ ID NO. 52365, respectively, or a homolog or a fragment thereof, is increased or generated, or if the activity glycogen synthase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 34 to 293-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a carbon dioxide concentrating mechanism protein, or if the activity of the polypeptide Sll1031, preferably represented by SEQ ID NO. 52635, or a homolog or fragment thereof, or if the activity of a polypeptide encoded by a nucleic acid molecule comprising the nucleic acid SEQ ID NO. 52634, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 52634 or polypeptide SEQ ID NO. 52635, respectively, or a homolog or a fragment thereof, is increased or generated, or if the activity carbon dioxide concentrating mechanism protein is increased or generated in a non-human organism, like a microorganism or a plant cell, plant or part thereof, especially with mitochondrial localization, whereby the respective line discloses in column 7 glutamate. For example, an increase of the glutamate of at least 1 percent, particularly in a range of 37 to 133-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a carbon dioxide concentrating mechanism protein, or if the activity of the polypeptide Sll1031, preferably represented by SEQ ID NO. 52635, or a homolog or fragment thereof, or if the activity of a polypeptide encoded by a nucleic acid molecule comprising the nucleic acid SEQ ID NO. 52634, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 52634 or polypeptide SEQ ID NO. 52635, respectively, or a homolog or a fragment thereof, is increased or generated, or if the activity carbon dioxide concentrating mechanism protein is increased or generated in a non-human organism, like a microorganism or a plant cell, plant or part thereof, especially with mitochondrial localization, whereby the respective line discloses in column 7 glutamine. For example, an increase of the glutamine of at least 1 percent, particularly in a range of 55 to 461-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a carbon dioxide concentrating mechanism protein, or if the activity of the polypeptide Sll1031, preferably represented by SEQ ID NO. 52635, or a homolog or fragment thereof, or if the activity of a polypeptide encoded by a nucleic acid molecule comprising the nucleic acid SEQ ID NO. 52634, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 52634 or polypeptide SEQ ID NO. 52635, respectively, or a homolog or a fragment thereof, is increased or generated, or if the activity carbon dioxide concentrating mechanism protein is increased or generated in a non-human organism, like a microorganism or a plant cell, plant or part thereof, especially with mitochondrial localization, whereby the respective line discloses in column 7 proline. For example, an increase of the proline of at least 1 percent, particularly in a range of 32 to 110-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a phosphoribosylformyl glycinamidine synthase subunit, or if the activity of the polypeptide Sll1056, preferably represented by SEQ ID NO. 52661, or a homolog or fragment thereof, or if the activity of a polypeptide encoded by a nucleic acid molecule comprising the nucleic acid SEQ ID NO. 52660, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 52660 or polypeptide SEQ ID NO. 52661, respectively, or a homolog or a fragment thereof, is increased or generated, or if the activity phosphoribosylformyl glycinamidine synthase subunit is increased or generated in a non-human organism, like a microorganism or a plant cell, plant or part thereof, especially with plastidic localization, whereby the respective line discloses in column 7 proline. For example, an increase of the proline of at least 1 percent, particularly in a range of 45 to 113-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a coproporphyrinogen oxidase, or if the activity of the polypeptide Sll1185, preferably represented by SEQ ID NO. 53190, or a homolog or fragment thereof, or if the activity of a polypeptide encoded by a nucleic acid molecule comprising the nucleic acid SEQ ID NO. 53189, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 53189 or polypeptide SEQ ID NO. 53190, respectively, or a homolog or a fragment thereof, is increased or generated, or if the activity coproporphyrinogen oxidase is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 33 to 55-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a glycogen (starch) synthase, or if the activity of the polypeptide Sll1393, preferably represented by SEQ ID NO. 53457, or a homolog or fragment thereof, or if the activity of a polypeptide encoded by a nucleic acid molecule comprising the nucleic acid SEQ ID NO. 53456, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 53456 or polypeptide SEQ ID NO. 53457, respectively, or a homolog or a fragment thereof, is increased or generated, or if the activity glycogen (starch) synthase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 49 to 73-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a glycogen (starch) synthase, or if the activity of the polypeptide Sll1393, preferably represented by SEQ ID NO. 53457, or a homolog or fragment thereof, or if the activity of a polypeptide encoded by a nucleic acid molecule comprising the nucleic acid SEQ ID NO. 53456, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 53456 or polypeptide SEQ ID NO. 53457, respectively, or a homolog or a fragment thereof, is increased or generated, or if the activity glycogen (starch) synthase is increased or generated in a non-human organism, like a microorganism or a plant cell, plant or part thereof, especially with non-targeted localization, For example, an increase of the proline of at least 1 percent, particularly in a range of 36 to 105 percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a fatty acid desaturase, or if the activity of the polypeptide Sll1441, preferably represented by SEQ ID NO. 53609, or a homolog or fragment thereof, or if the activity of a polypeptide encoded by a nucleic acid molecule comprising the nucleic acid SEQ ID NO. 53608, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 53608 or polypeptide SEQ ID NO. 53609, respectively, or a homolog or a fragment thereof, is increased or generated, or if the activity fatty acid desaturase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 37 to 37-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a CTP synthetase, or if the activity of the polypeptide Sll1443, preferably represented by SEQ ID NO. 53879, or a homolog or fragment thereof, or if the activity of a polypeptide encoded by a nucleic acid molecule comprising the nucleic acid SEQ ID NO. 53878, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 53878 or polypeptide SEQ ID NO. 53879, respectively, or a homolog or a fragment thereof, is increased or generated, or if the activity CTP synthetase is increased or generated in a non-human organism, like a microorganism or a plant cell, plant or part thereof, especially with mitochondrial localization, whereby the respective line discloses in column 7 glutamate. For example, an increase of the glutamate of at least 1 percent, particularly in a range of 33 to 61-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a nitrate/nitrite transport protein, or if the activity of the polypeptide Sll1450, preferably represented by SEQ ID NO. 54338, or a homolog or fragment thereof, or if the activity of a polypeptide encoded by a nucleic acid molecule comprising the nucleic acid SEQ ID NO. 54337, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 54337 or polypeptide SEQ ID NO. 54338, respectively, or a homolog or a fragment thereof, is increased or generated, or if the activity nitrate/nitrite transport protein is increased or generated in a non-human organism, like a microorganism or a plant cell, plant or part thereof, especially with mitochondrial localization, whereby the respective line discloses in column 7 glutamate. For example, an increase of the glutamate of at least 1 percent, particularly in a range of 34 to 90-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a CDP-diacylglycerol-glycerol-3-phosphate 3-phosphatidyltransferase, or if the activity of the polypeptide Sll1522, preferably represented by SEQ ID NO. 54453, or a homolog or fragment thereof, or if the activity of a polypeptide encoded by a nucleic acid molecule comprising the nucleic acid SEQ ID NO. 54452, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 54452 or polypeptide SEQ ID NO. 54453, respectively, or a homolog or a fragment thereof, is increased or generated, or if the activity CDP-diacylglycerol-glycerol-3-phosphate 3-phosphatidyltransferase is increased or generated in a non-human organism, like a microorganism or a plant cell, plant or part thereof, especially with plastidic localization, whereby the respective line discloses in column 7 glutamate. For example, an increase of the glutamate of at least 1 percent, particularly in a range of 63 to 154-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a glutathione S-transferase, or if the activity of the polypeptide Sll1545, preferably represented by SEQ ID NO. 11424, or a homolog or fragment thereof, or if the activity of a polypeptide encoded by a nucleic acid molecule comprising the nucleic acid SEQ ID NO. 11423, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 11423 or polypeptide SEQ ID NO. 11424, respectively, or a homolog or a fragment thereof, is increased or generated, or if the activity glutathione S-transferase is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 51 to 152-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a glutathione S-transferase, or if the activity of the polypeptide Sll1545, preferably represented by SEQ ID NO. 11424, or a homolog or fragment thereof, or if the activity of a polypeptide encoded by a nucleic acid molecule comprising the nucleic acid SEQ ID NO. 11423, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 11423 or polypeptide SEQ ID NO. 11424, respectively, or a homolog or a fragment thereof, is increased or generated, or if the activity glutathione S-transferase is increased or generated in a non-human organism, like a microorganism or a plant cell, plant or part thereof, especially with mitochondrial localization, whereby the respective line discloses in column 7 glutamine. For example, an increase of the glutamine of at least 1 percent, particularly in a range of 29 to 35-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a glutathione S-transferase, or if the activity of the polypeptide Sll1545, preferably represented by SEQ ID NO. 11424, or a homolog or fragment thereof, or if the activity of a polypeptide encoded by a nucleic acid molecule comprising the nucleic acid SEQ ID NO. 11423, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 11423 or polypeptide SEQ ID NO. 11424, respectively, or a homolog or a fragment thereof, is increased or generated, or if the activity glutathione S-transferase is increased or generated in a non-human organism, like a microorganism or a plant cell, plant or part thereof, especially with mitochondrial localization, whereby the respective line discloses in column 7 proline. For example, an increase of the proline of at least 1 percent, particularly in a range of 75 to 130-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a exopolyphosphatase, or if the activity of the polypeptide Sll1546, preferably represented by SEQ ID NO. 54805, or a homolog or fragment thereof, or if the activity of a polypeptide encoded by a nucleic acid molecule comprising the nucleic acid SEQ ID NO. 54804, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 54804 or polypeptide SEQ ID NO. 54805, respectively, or a homolog or a fragment thereof, is increased or generated, or if the activity exopolyphosphatase is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 64 to 260-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a 4-alpha-glucanotransferase, or if the activity of the polypeptide Sll1676, preferably represented by SEQ ID NO. 54898, or a homolog or fragment thereof, or if the activity of a polypeptide encoded by a nucleic acid molecule comprising the nucleic acid SEQ ID NO. 54897, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 54897 or polypeptide SEQ ID NO. 54898, respectively, or a homolog or a fragment thereof, is increased or generated, or if the activity 4-alpha-glucanotransferase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 44 to 186-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a alanine dehydrogenase, or if the activity of the polypeptide Sll1682, preferably represented by SEQ ID NO. 55064, or a homolog or fragment thereof, or if the activity of a polypeptide encoded by a nucleic acid molecule comprising the nucleic acid SEQ ID NO. 55063, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 55063 or polypeptide SEQ ID NO. 55064, respectively, or a homolog or a fragment thereof, is increased or generated, or if the activity alanine dehydrogenase is increased or generated in a non-human organism, like a microorganism or a plant cell, plant or part thereof, especially with plastidic localization, whereby the respective line discloses in column 7 proline. For example, an increase of the proline of at least 1 percent, particularly in a range of 76 to 272-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a sll1761-protein, or if the activity of the polypeptide Sll1761, preferably represented by SEQ ID NO. 55380, or a homolog or fragment thereof, or if the activity of a polypeptide encoded by a nucleic acid molecule comprising the nucleic acid SEQ ID NO. 55379, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 55379 or polypeptide SEQ ID NO. 55380, respectively, or a homolog or a fragment thereof, is increased or generated, or if the activity sll1761-protein is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 69 to 387-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a sll761-protein, or if the activity of the polypeptide Sll1761, preferably represented by SEQ ID NO. 55380, or a homolog or fragment thereof, or if the activity of a polypeptide encoded by a nucleic acid molecule comprising the nucleic acid SEQ ID NO. 55379, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 55379 or polypeptide SEQ ID NO. 55380, respectively, or a homolog or a fragment thereof, is increased or generated, or if the activity sll1761-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 36 to 269-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a coproporphyrinogen III oxidase, or if the activity of the polypeptide Sll1917, preferably represented by SEQ ID NO. 11472, or a homolog or fragment thereof, or if the activity of a polypeptide encoded by a nucleic acid molecule comprising the nucleic acid SEQ ID NO. 11471, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 11471 or polypeptide SEQ ID NO. 11472, respectively, or a homolog or a fragment thereof, is increased or generated, or if the activity coproporphyrinogen III oxidase is increased or generated in a non-human organism, like a microorganism or a plant cell, plant or part thereof, especially with mitochondrial localization, whereby the respective line discloses in column 7 glutamate. For example, an increase of the glutamate of at least 1 percent, particularly in a range of 42 to 167-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a coproporphyrinogen III oxidase, or if the activity of the polypeptide Sll1917, preferably represented by SEQ ID NO. 11472, or a homolog or fragment thereof, or if the activity of a polypeptide encoded by a nucleic acid molecule comprising the nucleic acid SEQ ID NO. 11471, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 11471 or polypeptide SEQ ID NO. 11472, respectively, or a homolog or a fragment thereof, is increased or generated, or if the activity coproporphyrinogen III oxidase is increased or generated in a non-human organism, like a microorganism or a plant cell, plant or part thereof, especially with mitochondrial localization, whereby the respective line discloses in column 7 glutamine. For example, an increase of the glutamine of at least 1 percent, particularly in a range of 47 to 318-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a coproporphyrinogen III oxidase, or if the activity of the polypeptide Sll1917, preferably represented by SEQ ID NO. 11472, or a homolog or fragment thereof, or if the activity of a polypeptide encoded by a nucleic acid molecule comprising the nucleic acid SEQ ID NO. 11471, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 11471 or polypeptide SEQ ID NO. 11472, respectively, or a homolog or a fragment thereof, is increased or generated, or if the activity coproporphyrinogen III oxidase is increased or generated in a non-human organism, like a microorganism or a plant cell, plant or part thereof, especially with mitochondrial localization, whereby the respective line discloses in column 7 proline. For example, an increase of the proline of at least 1 percent, particularly in a range of 34 to 176-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a cation-transporting ATPase, or if the activity of the polypeptide Sll1920, preferably represented by SEQ ID NO. 55386, or a homolog or fragment thereof, or if the activity of a polypeptide encoded by a nucleic acid molecule comprising the nucleic acid SEQ ID NO. 55385, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 55385 or polypeptide SEQ ID NO. 55386, respectively, or a homolog or a fragment thereof, is increased or generated, or if the activity cation-transporting ATPase is increased or generated in a non-human organism, like a microorganism or a plant cell, plant or part thereof, especially with mitochondrial localization, whereby the respective line discloses in column 7 glutamate. For example, an increase of the glutamate of at least 1 percent, particularly in a range of 46 to 113-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a cationtransporting ATPase, or if the activity of the polypeptide Sll1920, preferably represented by SEQ ID NO. 55386, or a homolog or fragment thereof, or if the activity of a polypeptide encoded by a nucleic acid molecule comprising the nucleic acid SEQ ID NO. 55385, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 55385 or polypeptide SEQ ID NO. 55386, respectively, or a homolog or a fragment thereof, is increased or generated, or if the activity cation-transporting ATPase is increased or generated in a non-human organism, like a microorganism or a plant cell, plant or part thereof, especially with mitochondrial localization, whereby the respective line discloses in column 7 glutamine. For example, an increase of the glutamine of at least 1 percent, particularly in a range of 44 to 372-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a glycosidase, or if the activity of the polypeptide Slr0237, preferably represented by SEQ ID NO. 55772, or a homolog or fragment thereof, or if the activity of a polypeptide encoded by a nucleic acid molecule comprising the nucleic acid SEQ ID NO. 55771, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 55771 or polypeptide SEQ ID NO. 55772, respectively, or a homolog or a fragment thereof, is increased or generated, or if the activity glycosidase is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 84 to 104-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a glycosidase, or if the activity of the polypeptide Slr0237, preferably represented by SEQ ID NO. 55772, or a homolog or fragment thereof, or if the activity of a polypeptide encoded by a nucleic acid molecule comprising the nucleic acid SEQ ID NO. 55771, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 55771 or polypeptide SEQ ID NO. 55772, respectively, or a homolog or a fragment thereof, is increased or generated, or if the activity glycosidase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 30 to 40-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a phosphoribosylglycinamide formyltransferase, or if the activity of the polypeptide Slr0477, preferably represented by SEQ ID NO. 55979, or a homolog or fragment thereof, or if the activity of a polypeptide encoded by a nucleic acid molecule comprising the nucleic acid SEQ ID NO. 55978, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 55978 or polypeptide SEQ ID NO. 55979, respectively, or a homolog or a fragment thereof, is increased or generated, or if the activity phosphoribosylglycinamide formyltransferase is increased or generated in a non-human organism, like a microorganism or a plant cell, plant or part thereof, especially with plastidic localization, whereby the respective line discloses in column 7 proline. For example, an increase of the proline of at least 1 percent, particularly in a range of 42 to 86-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a bifunctional purine biosynthesis protein, or if the activity of the polypeptide Slr0597, preferably represented by SEQ ID NO. 56154, or a homolog or fragment thereof, or if the activity of a polypeptide encoded by a nucleic acid molecule comprising the nucleic acid SEQ ID NO. 56153, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 56153 or polypeptide SEQ ID NO. 56154, respectively, or a homolog or a fragment thereof, is increased or generated, or if the activity bifunctional purine biosynthesis protein is increased or generated in a non-human organism, like a microorganism or a plant cell, plant or part thereof, especially with plastidic localization, whereby the respective line discloses in column 7 glutamate. For example, an increase of the glutamate of at least 1 percent, particularly in a range of 43 to 100-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a slr0600-protein, or if the activity of the polypeptide Slr0600, preferably represented by SEQ ID NO. 56515, or a homolog or fragment thereof, or if the activity of a polypeptide encoded by a nucleic acid molecule comprising the nucleic acid SEQ ID NO. 56514, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 56514 or polypeptide SEQ ID NO. 56515, respectively, or a homolog or a fragment thereof, is increased or generated, or if the activity slr0600-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 43 to 60-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a pyrroline carboxylate reductase, or if the activity of the polypeptide Slr0661, preferably represented by SEQ ID NO. 56577, or a homolog or fragment thereof, or if the activity of a polypeptide encoded by a nucleic acid molecule comprising the nucleic acid SEQ ID NO. 56576, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 56576 or polypeptide SEQ ID NO. 56577, respectively, or a homolog or a fragment thereof, is increased or generated, or if the activity pyrroline carboxylate reductase is increased or generated in a non-human organism, like a microorganism or a plant cell, plant or part thereof, especially with mitochondrial localization, whereby the respective line discloses in column 7 proline. For example, an increase of the proline of at least 1 percent, particularly in a range of 45 to 85-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a Glu/Leu/Phe/Val dehydrogenase, or if the activity of the polypeptide Slr0710, preferably represented by SEQ ID NO. 56895, or a homolog or fragment thereof, or if the activity of a polypeptide encoded by a nucleic acid molecule comprising the nucleic acid SEQ ID NO. 56894, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 56894 or polypeptide SEQ ID NO. 56895, respectively, or a homolog or a fragment thereof, is increased or generated, or if the activity Glu/Leu/Phe/Val dehydrogenase is increased or generated in a non-human organism, like a microorganism or a plant cell, plant or part thereof, especially with mitochondrial localization, whereby the respective line discloses in column 7 arginine. For example, an increase of the arginine of at least 1 percent, particularly in a range of 47 to 175-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a geranylgeranyl pyrophosphate synthase, or if the activity of the polypeptide Slr0739, preferably represented by SEQ ID NO. 57236, or a homolog or fragment thereof, or if the activity of a polypeptide encoded by a nucleic acid molecule comprising the nucleic acid SEQ ID NO. 57235, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 57235 or polypeptide SEQ ID NO. 57236, respectively, or a homolog or a fragment thereof, is increased or generated, or if the activity geranylgeranyl pyrophosphate synthase is increased or generated in a non-human organism, like a microorganism or a plant cell, plant or part thereof, especially with plastidic localization, whereby the respective line discloses in column 7 arginine. For example, an increase of the arginine of at least 1 percent, particularly in a range of 347 to 910-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a geranylgeranyl pyrophosphate synthase, or if the activity of the polypeptide Slr0739, preferably represented by SEQ ID NO. 57236, or a homolog or fragment thereof, or if the activity of a polypeptide encoded by a nucleic acid molecule comprising the nucleic acid SEQ ID NO. 57235, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 57235 or polypeptide SEQ ID NO. 57236, respectively, or a homolog or a fragment thereof, is increased or generated, or if the activity geranylgeranyl pyrophosphate synthase is increased or generated in a non-human organism, like a microorganism or a plant cell, plant or part thereof, especially with plastidic localization, whereby the respective line discloses in column 7 glutamine. For example, an increase of the glutamine of at least 1 percent, particularly in a range of 46 to 519-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a geranylgeranyl pyrophosphate synthase, or if the activity of the polypeptide Slr0739, preferably represented by SEQ ID NO. 57236, or a homolog or fragment thereof, or if the activity of a polypeptide encoded by a nucleic acid molecule comprising the nucleic acid SEQ ID NO. 57235, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 57235 or polypeptide SEQ ID NO. 57236, respectively, or a homolog or a fragment thereof, is increased or generated, or if the activity geranylgeranyl pyrophosphate synthase is increased or generated in a non-human organism, like a microorganism or a plant cell, plant or part thereof, especially with plastidic localization, whereby the respective line discloses in column 7 proline. For example, an increase of the proline of at least 1 percent, particularly in a range of 105 to 801-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a circadian clock protein, or if the activity of the polypeptide Slr0756, preferably represented by SEQ ID NO. 57664, or a homolog or fragment thereof, or if the activity of a polypeptide encoded by a nucleic acid molecule comprising the nucleic acid SEQ ID NO. 57663, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 57663 or polypeptide SEQ ID NO. 57664, respectively, or a homolog or a fragment thereof, is increased or generated, or if the activity circadian clock protein is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 48 to 280-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a circadian clock protein, or if the activity of the polypeptide Slr0756, preferably represented by SEQ ID NO. 57664, or a homolog or fragment thereof, or if the activity of a polypeptide encoded by a nucleic acid molecule comprising the nucleic acid SEQ ID NO. 57663, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 57663 or polypeptide SEQ ID NO. 57664, respectively, or a homolog or a fragment thereof, is increased or generated, or if the activity circadian clock protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 32 to 91-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a amine oxidase, or if the activity of the polypeptide Slr0782, preferably represented by SEQ ID NO. 57680, or a homolog or fragment thereof, or if the activity of a polypeptide encoded by a nucleic acid molecule comprising the nucleic acid SEQ ID NO. 57679, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 57679 or polypeptide SEQ ID NO. 57680, respectively, or a homolog or a fragment thereof, is increased or generated, or if the activity amine oxidase is increased or generated in a non-human organism, like a microorganism or a plant cell, plant or part thereof, especially with mitochondrial localization, whereby the respective line discloses in column 7 proline. For example, an increase of the proline of at least 1 percent, particularly in a range of 43 to 94-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a dihydrolipoamide dehydrogenase, or if the activity of the polypeptide Slr1096, preferably represented by SEQ ID NO. 57735, or a homolog or fragment thereof, or if the activity of a polypeptide encoded by a nucleic acid molecule comprising the nucleic acid SEQ ID NO. 57734, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 57734 or polypeptide SEQ ID NO. 57735, respectively, or a homolog or a fragment thereof, is increased or generated, or if the activity dihydrolipoamide dehydrogenase is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 41 to 79-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a gamma-glutamyltranspeptidase, or if the activity of the polypeptide Slr1269, preferably represented by SEQ ID NO. 58059, or a homolog or fragment thereof, or if the activity of a polypeptide encoded by a nucleic acid molecule comprising the nucleic acid SEQ ID NO. 58058, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 58058 or polypeptide SEQ ID NO. 58059, respectively, or a homolog or a fragment thereof, is increased or generated, or if the activity gamma-glutamyltranspeptidase is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 65 to 209-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a gamma-polypeptideglutamyltranspeptidase, or if the activity of the polypeptide Slr1269, preferably represented by SEQ ID NO. 58059, or a homolog or fragment thereof, or if the activity of a polypeptide encoded by a nucleic acid molecule comprising the nucleic acid SEQ ID NO. 58058, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 58058 or polypeptide SEQ ID NO. 58059, respectively, or a homolog or a fragment thereof, is increased or generated, or if the activity gamma-glutamyltranspeptidase is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 63 to 150-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a arginine decarboxylase, or if the activity of the polypeptide Slr1312, preferably represented by SEQ ID NO. 58325, or a homolog or fragment thereof, or if the activity of a polypeptide encoded by a nucleic acid molecule comprising the nucleic acid SEQ ID NO. 58324, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 58324 or polypeptide SEQ ID NO. 58325, respectively, or a homolog or a fragment thereof, is increased or generated, or if the activity arginine decarboxylase is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 52 to 306-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a CDP-diglyceride synthetase, or if the activity of the polypeptide Slr1369, preferably represented by SEQ ID NO. 58473, or a homolog or fragment thereof, or if the activity of a polypeptide encoded by a nucleic acid molecule comprising the nucleic acid SEQ ID NO. 58472, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 58472 or polypeptide SEQ ID NO. 58473, respectively, or a homolog or a fragment thereof, is increased or generated, or if the activity CDP-diglyceride synthetase is increased or generated in a non-human organism, like a microorganism or a plant cell, plant or part thereof, especially with mitochondrial localization, whereby the respective line discloses in column 7 glutamate. For example, an increase of the glutamate of at least 1 percent, particularly in a range of 39 to 86-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a carbohydrate kinase, or if the activity of the polypeptide Slr1420, preferably represented by SEQ ID NO. 58591, or a homolog or fragment thereof, or if the activity of a polypeptide encoded by a nucleic acid molecule comprising the nucleic acid SEQ ID NO. 58590, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 58590 or polypeptide SEQ ID NO. 58591, respectively, or a homolog or a fragment thereof, is increased or generated, or if the activity carbohydrate kinase is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 35 to 196-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a iron(III) dicitrate-binding protein, or if the activity of the polypeptide Slr1492, preferably represented by SEQ ID NO. 58669, or a homolog or fragment thereof, or if the activity of a polypeptide encoded by a nucleic acid molecule comprising the nucleic acid SEQ ID NO. 58668, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 58668 or polypeptide SEQ ID NO. 58669, respectively, or a homolog or a fragment thereof, is increased or generated, or if the activity iron(III) dicitrate-binding protein is increased or generated in a non-human organism, like a microorganism or a plant cell, plant or part thereof, especially with mitochondrial localization, whereby the respective line discloses in column 7 glutamine. For example, an increase of the glutamine of at least 1 percent, particularly in a range of 30 to 46-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a Photosystem I reaction center subunit XI, or if the activity of the polypeptide Slr1655, preferably represented by SEQ ID NO. 12071, or a homolog or fragment thereof, or if the activity of a polypeptide encoded by a nucleic acid molecule comprising the nucleic acid SEQ ID NO. 12070, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 12070 or polypeptide SEQ ID NO. 12071, respectively, or a homolog or a fragment thereof, is increased or generated, or if the activity Photosystem I reaction center subunit XI is increased or generated in a non-human organism, like a microorganism or a plant cell, plant or part thereof, especially with mitochondrial localization, whereby the respective line discloses in column 7 proline. For example, an increase of the proline of at least 1 percent, particularly in a range of 40 to 164-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a photosystem II protein, or if the activity of the polypeptide Slr1739, preferably represented by SEQ ID NO. 58732, or a homolog or fragment thereof, or if the activity of a polypeptide encoded by a nucleic acid molecule comprising the nucleic acid SEQ ID NO. 58731, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 58731 or polypeptide SEQ ID NO. 58732, respectively, or a homolog or a fragment thereof, is increased or generated, or if the activity photosystem II protein is increased or generated in a non-human organism, like a microorganism or a plant cell, plant or part thereof, especially with plastidic localization, whereby the respective line discloses in column 7 arginine. For example, an increase of the arginine of at least 1 percent, particularly in a range of 99 to 193-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a photosystem II protein, or if the activity of the polypeptide Slr1739, preferably represented by SEQ ID NO. 58732, or a homolog or fragment thereof, or if the activity of a polypeptide encoded by a nucleic acid molecule comprising the nucleic acid SEQ ID NO. 58731, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 58731 or polypeptide SEQ ID NO. 58732, respectively, or a homolog or a fragment thereof, is increased or generated, or if the activity photosystem II protein is increased or generated in a non-human organism, like a microorganism or a plant cell, plant or part thereof, especially with plastidic localization, whereby the respective line discloses in column 7 glutamine. For example, an increase of the glutamine of at least 1 percent, particularly in a range of 42 to 229-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a glutamine amidotransferase, or if the activity of the polypeptide Slr1742, preferably represented by SEQ ID NO. 58752, or a homolog or fragment thereof, or if the activity of a polypeptide encoded by a nucleic acid molecule comprising the nucleic acid SEQ ID NO. 58751, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 58751 or polypeptide SEQ ID NO. 58752, respectively, or a homolog or a fragment thereof, is increased or generated, or if the activity glutamine amidotransferase is increased or generated in a non-human organism, like a microorganism or a plant cell, plant or part thereof, especially with mitochondrial localization, whereby the respective line discloses in column 7 proline. For example, an increase of the proline of at least 1 percent, particularly in a range of 49 to 75-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a glycerol-3-phosphate dehydrogenase, or if the activity of the polypeptide Slr1755, preferably represented by SEQ ID NO. 58824, or a homolog or fragment thereof, or if the activity of a polypeptide encoded by a nucleic acid molecule comprising the nucleic acid SEQ ID NO. 58823, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 58823 or polypeptide SEQ ID NO. 58824, respectively, or a homolog or a fragment thereof, is increased or generated, or if the activity glycerol-3-phosphate dehydrogenase is increased or generated in a non-human organism, like a microorganism or a plant cell, plant or part thereof, especially with mitochondrial localization, whereby the respective line discloses in column 7 glutamate. For example, an increase of the glutamate of at least 1 percent, particularly in a range of 40 to 62 percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a phosphoadenosine phosphosulfate reductase, or if the activity of the polypeptide Slr1791, preferably represented by SEQ ID NO. 12141, or a homolog or fragment thereof, or if the activity of a polypeptide encoded by a nucleic acid molecule comprising the nucleic acid SEQ ID NO. 12140, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 12140 or polypeptide SEQ ID NO. 12141, respectively, or a homolog or a fragment thereof, is increased or generated, or if the activity phosphoadenosine phosphosulfate reductase is increased or generated in a non-human organism, like a microorganism or a plant cell, plant or part thereof, especially with plastidic localization, whereby the respective line discloses in column 7 proline. For example, an increase of the proline of at least 1 percent, particularly in a range of 32 to 53-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a riboflavin biosynthesis protein, or if the activity of the polypeptide Slr1882, preferably represented by SEQ ID NO. 59042, or a homolog or fragment thereof, or if the activity of a polypeptide encoded by a nucleic acid molecule comprising the nucleic acid SEQ ID NO. 59041, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 59041 or polypeptide SEQ ID NO. 59042, respectively, or a homolog or a fragment thereof, is increased or generated, or if the activity riboflavin biosynthesis protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 34 to 114-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a malonyl CoA-acyl carrier protein transacylase, or if the activity of the polypeptide Slr2023, preferably represented by SEQ ID NO. 59166, or a homolog or fragment thereof, or if the activity of a polypeptide encoded by a nucleic acid molecule comprising the nucleic acid SEQ ID NO. 59165, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 59165 or polypeptide SEQ ID NO. 59166, respectively, or a homolog or a fragment thereof, is increased or generated, or if the activity malonyl CoA-acyl carrier protein transacylase is increased or generated in a non-human organism, like a microorganism or a plant cell, plant or part thereof, especially with plastidic localization, whereby the respective line discloses in column 7 glutamate. For example, an increase of the glutamate of at least 1 percent, particularly in a range of 123 to 185-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a malonyl CoA-acyl carrier protein transacylase, or if the activity of the polypeptide Slr2023, preferably represented by SEQ ID NO. 59166, or a homolog or fragment thereof, or if the activity of a polypeptide encoded by a nucleic acid molecule comprising the nucleic acid SEQ ID NO. 59165, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 59165 or polypeptide SEQ ID NO. 59166, respectively, or a homolog or a fragment thereof, is increased or generated, or if the activity malonyl CoA-acyl carrier protein transacylase is increased or generated in a non-human organism, like a microorganism or a plant cell, plant or part thereof, especially with plastidic localization, whereby the respective line discloses in column 7 glutamine. For example, an increase of the glutamine of at least 1 percent, particularly in a range of 70 to 355-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a short-chain alcohol dehydrogenase family, or if the activity of the polypeptide Slr2124, preferably represented by SEQ ID NO. 59371, or a homolog or fragment thereof, or if the activity of a polypeptide encoded by a nucleic acid molecule comprising the nucleic acid SEQ ID NO. 59370, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 59370 or polypeptide SEQ ID NO. 59371, respectively, or a homolog or a fragment thereof, is increased or generated, or if the activity short-chain alcohol dehydrogenase family is increased or generated in a non-human organism, like a microorganism or a plant cell, plant or part thereof, especially with plastidic localization, whereby the respective line discloses in column 7 proline. For example, an increase of the proline of at least 1 percent, particularly in a range of 53 to 228-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a Sec-independent protein translocase subunit, or if the activity of the polypeptide TTC0019, preferably represented by SEQ ID NO. 12699, or a homolog or fragment thereof, or if the activity of a polypeptide encoded by a nucleic acid molecule comprising the nucleic acid SEQ ID NO. 12698, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 12698 or polypeptide SEQ ID NO. 12699, respectively, or a homolog or a fragment thereof, is increased or generated, or if the activity Sec-independent protein translocase subunit is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 72 to 162-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a Sec-independent protein translocase subunit, or if the activity of the polypeptide TTC0019, preferably represented by SEQ ID NO. 12699, or a homolog or fragment thereof, or if the activity of a polypeptide encoded by a nucleic acid molecule comprising the nucleic acid SEQ ID NO. 12698, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 12698 or polypeptide SEQ ID NO. 12699, respectively, or a homolog or a fragment thereof, is increased or generated, or if the activity Sec-independent protein translocase subunit is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 45 to 228-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a cell division protein, or if the activity of the polypeptide TTC0035, preferably represented by SEQ ID NO. 60302, or a homolog or fragment thereof, or if the activity of a polypeptide encoded by a nucleic acid molecule comprising the nucleic acid SEQ ID NO. 60301, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 60301 or polypeptide SEQ ID NO. 60302, respectively, or a homolog or a fragment thereof, is increased or generated, or if the activity cell division protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 44 to 145-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a branched-chain amino acid ABC transporter permease protein, or if the activity of the polypeptide TTC0216, preferably represented by SEQ ID NO. 60860, or a homolog or fragment thereof, or if the activity of a polypeptide encoded by a nucleic acid molecule comprising the nucleic acid SEQ ID NO. 60859, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 60859 or polypeptide SEQ ID NO. 60860, respectively, or a homolog or a fragment thereof, is increased or generated, or if the activity branched-chain amino acid ABC transporter permease protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 37 to 137-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a amino acid ABC transporter permease protein, or if the activity of the polypeptide TTC0337, preferably represented by SEQ ID NO. 61071, or a homolog or fragment thereof, or if the activity of a polypeptide encoded by a nucleic acid molecule comprising the nucleic acid SEQ ID NO. 61070, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 61070 or polypeptide SEQ ID NO. 61071, respectively, or a homolog or a fragment thereof, is increased or generated, or if the activity amino acid ABC transporter permease protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 37 to 74 percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a TTC0768-protein, or if the activity of the polypeptide TTC0768, preferably represented by SEQ ID NO. 61533, or a homolog or fragment thereof, or if the activity of a polypeptide encoded by a nucleic acid molecule comprising the nucleic acid SEQ ID NO. 61532, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 61532 or polypeptide SEQ ID NO. 61533, respectively, or a homolog or a fragment thereof, is increased or generated, or if the activity TTC0768-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 48 to 77-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a metal-dependent hydrolase, or if the activity of the polypeptide TTC0917, preferably represented by SEQ ID NO. 61554, or a homolog or fragment thereof, or if the activity of a polypeptide encoded by a nucleic acid molecule comprising the nucleic acid SEQ ID NO. 61553, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 61553 or polypeptide SEQ ID NO. 61554, respectively, or a homolog or a fragment thereof, is increased or generated, or if the activity metal-dependent hydrolase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 58 to 79-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a multiple antibiotic resistance protein, or if the activity of the polypeptide TTC1193, preferably represented by SEQ ID NO. 61724, or a homolog or fragment thereof, or if the activity of a polypeptide encoded by a nucleic acid molecule comprising the nucleic acid SEQ ID NO. 61723, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 61723 or polypeptide SEQ ID NO. 61724, respectively, or a homolog or a fragment thereof, is increased or generated, or if the activity multiple antibiotic resistance protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 35 to 73-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a TTC1386-protein, or if the activity of the polypeptide TTC1386, preferably represented by SEQ ID NO. 62080, or a homolog or fragment thereof, or if the activity of a polypeptide encoded by a nucleic acid molecule comprising the nucleic acid SEQ ID NO. 62079, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 62079 or polypeptide SEQ ID NO. 62080, respectively, or a homolog or a fragment thereof, is increased or generated, or if the activity TTC1386-protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 66 to 106-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a homocitrate synthase, or if the activity of the polypeptide TTC1550, preferably represented by SEQ ID NO. 12975, or a homolog or fragment thereof, or if the activity of a polypeptide encoded by a nucleic acid molecule comprising the nucleic acid SEQ ID NO. 12974, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 12974 or polypeptide SEQ ID NO. 12975, respectively, or a homolog or a fragment thereof, is increased or generated, or if the activity homocitrate synthase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 56 to 248-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a homocitrate synthase, or if the activity of the polypeptide TTC1550, preferably represented by SEQ ID NO. 12975, or a homolog or fragment thereof, or if the activity of a polypeptide encoded by a nucleic acid molecule comprising the nucleic acid SEQ ID NO. 12974, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 12974 or polypeptide SEQ ID NO. 12975, respectively, or a homolog or a fragment thereof, is increased or generated, or if the activity homocitrate synthase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 54 to 320-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a oxidoreductase subunit, or if the activity of the polypeptide TTC1918, preferably represented by SEQ ID NO. 62161, or a homolog or fragment thereof, or if the activity of a polypeptide encoded by a nucleic acid molecule comprising the nucleic acid SEQ ID NO. 62160, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 62160 or polypeptide SEQ ID NO. 62161, respectively, or a homolog or a fragment thereof, is increased or generated, or if the activity oxidoreductase subunit is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 43 to 128-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a oxidoreductase subunit, or if the activity of the polypeptide TTC1918, preferably represented by SEQ ID NO. 62161, or a homolog or fragment thereof, or if the activity of a polypeptide encoded by a nucleic acid molecule comprising the nucleic acid SEQ ID NO. 62160, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 62160 or polypeptide SEQ ID NO. 62161, respectively, or a homolog or a fragment thereof, is increased or generated, or if the activity oxidoreductase subunit is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 42 to 139-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a XM_{—}473199-protein, or if the activity of the polypeptide XM_{—}473199, preferably represented by SEQ ID NO. 62245, or a homolog or fragment thereof, or if the activity of a polypeptide encoded by a nucleic acid molecule comprising the nucleic acid SEQ ID NO. 62244, preferably the coding region thereof, or a homolog or fragment thereof, e.g. derived from Oryza sativa, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 62244 or polypeptide SEQ ID NO. 62245, respectively, or a homolog or a fragment thereof, is increased or generated, or if the activity XM_{—}473199-protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 45 to 75-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a XM_{—}473199-protein, or if the activity of the polypeptide XM_{—}473199, preferably represented by SEQ ID NO. 62245, or a homolog or fragment thereof, or if the activity of a polypeptide encoded by a nucleic acid molecule comprising the nucleic acid SEQ ID NO. 62244, preferably the coding region thereof, or a homolog or fragment thereof, e.g. derived from Oryza sativa, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 62244 or polypeptide SEQ ID NO. 62245, respectively, or a homolog or a fragment thereof, is increased or generated, or if the activity XM_{—}473199-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 62 to 173-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a transcription factor, or if the activity of the polypeptide Ybl021 c, preferably represented by SEQ ID NO. 62525, or a homolog or fragment thereof, or if the activity of a polypeptide encoded by a nucleic acid molecule comprising the nucleic acid SEQ ID NO. 62524, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 62524 or polypeptide SEQ ID NO. 62525, respectively, or a homolog or a fragment thereof, is increased or generated, or if the activity transcription factor is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 35 to 804-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a cell division controt protein, or if the activity of the polypeptide Ybr160w, preferably represented by SEQ ID NO. 62718, or a homolog or fragment thereof, or if the activity of a polypeptide encoded by a nucleic acid molecule comprising the nucleic acid SEQ ID NO. 62717, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 62717 or polypeptide SEQ ID NO. 62718, respectively, or a homolog or a fragment thereof, is increased or generated, or if the activity cell division control protein is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 37 to 118-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a cell division control protein, or if the activity of the polypeptide Ybr160w, preferably represented by SEQ ID NO. 62718, or a homolog or fragment thereof, or if the activity of a polypeptide encoded by a nucleic acid molecule comprising the nucleic acid SEQ ID NO. 62717, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 62717 or polypeptide SEQ ID NO. 62718, respectively, or a homolog or a fragment thereof, is increased or generated, or if the activity cell division control protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 36 to 323-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a YCL026C-A-protein, or if the activity of the polypeptide Yc1026c-a, preferably represented by SEQ ID NO. 63168, or a homolog or fragment thereof, or if the activity of a polypeptide encoded by a nucleic acid molecule comprising the nucleic acid SEQ ID NO. 63167, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 63167 or polypeptide SEQ ID NO. 63168, respectively, or a homolog or a fragment thereof, is increased or generated, or if the activity YCL026C-A-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 51 to 68-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a ycr102c-protein, or if the activity of the polypeptide Ycr102c, preferably represented by SEQ ID NO. 63265, or a homolog or fragment thereof, or if the activity of a polypeptide encoded by a nucleic acid molecule comprising the nucleic acid SEQ ID NO. 63264, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 63264 or polypeptide SEQ ID NO. 63265, respectively, or a homolog or a fragment thereof, is increased or generated, or if the activity ycr102c-protein is increased or generated in a non-human organism, like a microorganism or a plant cell, plant or part thereof, especially with plastidic localization, whereby the respective line discloses in column 7 arginine. For example, an increase of the arginine of at least 1 percent, particularly in a range of 78 to 107-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a protein kinase, or if the activity of the polypeptide Ydl168w, preferably represented by SEQ ID NO. 14303, or a homolog or fragment thereof, or if the activity of a polypeptide encoded by a nucleic acid molecule comprising the nucleic acid SEQ ID NO. 14302, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 14302 or polypeptide SEQ ID NO. 14303, respectively, or a homolog or a fragment thereof, is increased or generated, or if the activity protein kinase is increased or generated in a non-human organism, like a microorganism or a plant cell, plant or part thereof, especially with plastidic localization, whereby the respective line discloses in column 7 proline. For example, an increase of the proline of at least 1 percent, particularly in a range of 42 to 99-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a coproporphyrinogen III oxidase, or if the activity of the polypeptide Ydr044w, preferably represented by SEQ ID NO. 63335, or a homolog or fragment thereof, or if the activity of a polypeptide encoded by a nucleic acid molecule comprising the nucleic acid SEQ ID NO. 63334, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 63334 or polypeptide SEQ ID NO. 63335, respectively, or a homolog or a fragment thereof, is increased or generated, or if the activity coproporphyrinogen III oxidase is increased or generated in a non-human organism, like a microorganism or a plant cell, plant or part thereof, especially with plastidic localization, whereby the respective line discloses in column 7 proline. For example, an increase of the proline of at least 1 percent, particularly in a range of 47 to 228-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a branched-chain amino acid permease, or if the activity of the polypeptide Ydr046c, preferably represented by SEQ ID NO. 63545, or a homolog or fragment thereof, or if the activity of a polypeptide encoded by a nucleic acid molecule comprising the nucleic acid SEQ ID NO. 63544, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 63544 or polypeptide SEQ ID NO. 63545, respectively, or a homolog or a fragment thereof, is increased or generated, or if the activity branched-chain amino acid permease is increased or generated in a non-human organism, like a microorganism or a plant cell, plant or part thereof, especially with plastidic localization, whereby the respective line discloses in column 7 proline. For example, an increase of the proline of at least 1 percent, particularly in a range of 66 to 75-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a peroxisome assembly protein, or if the activity of the polypeptide Ydr265w, preferably represented by SEQ ID NO. 63666, or a homolog or fragment thereof, or if the activity of a polypeptide encoded by a nucleic acid molecule comprising the nucleic acid SEQ ID NO. 63665, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 63665 or polypeptide SEQ ID NO. 63666, respectively, or a homolog or a fragment thereof, is increased or generated, or if the activity peroxisome assembly protein is increased or generated in a non-human organism, like a microorganism or a plant cell, plant or part thereof, especially with plastidic localization, whereby the respective line discloses in column 7 arginine. For example, an increase of the arginine of at least 1 percent, particularly in a range of 88 to 276-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a ydr338c-protein, or if the activity of the polypeptide Ydr338c, preferably represented by SEQ ID NO. 63714, or a homolog or fragment thereof, or if the activity of a polypeptide encoded by a nucleic acid molecule comprising the nucleic acid SEQ ID NO. 63713, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 63713 or polypeptide SEQ ID NO. 63714, respectively, or a homolog or a fragment thereof, is increased or generated, or if the activity ydr338c-protein is increased or generated in a non-human organism, like a microorganism or a plant cell, plant or part thereof, especially with plastidic localization, whereby the respective line discloses in column 7 arginine. For example, an increase of the arginine of at least 1 percent, particularly in a range of 67 to 165-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a yer014w-protein, or if the activity of the polypeptide Yer014w, preferably represented by SEQ ID NO. 63746, or a homolog or fragment thereof, or if the activity of a polypeptide encoded by a nucleic acid molecule comprising the nucleic acid SEQ ID NO. 63745, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 63745 or polypeptide SEQ ID NO. 63746, respectively, or a homolog or a fragment thereof, is increased or generated, or if the activity yer014w-protein is increased or generated in a non-human organism, like a microorganism or a plant cell, plant or part thereof, especially with plastidic localization, whereby the respective line discloses in column 7 glutamine. For example, an increase of the glutamine of at least 1 percent, particularly in a range of 74 to 186-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a yer106w-protein, or if the activity of the polypeptide Yer106w, preferably represented by SEQ ID NO. 63804, or a homolog or fragment thereof, or if the activity of a polypeptide encoded by a nucleic acid molecule comprising the nucleic acid SEQ ID NO. 63803, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 63803 or polypeptide SEQ ID NO. 63804, respectively, or a homolog or a fragment thereof, is increased or generated, or if the activity yer106w-protein is increased or generated in a non-human organism, like a microorganism or a plant cell, plant or part thereof, especially with plastidic localization, whereby the respective line discloses in column 7 glutamine. For example, an increase of the glutamine of at least 1 percent, particularly in a range of 43 to 118-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a molecular chaperone portein, or if the activity of the polypeptide Yfl016c, preferably represented by SEQ ID NO. 63808, or a homolog or fragment thereof, or if the activity of a polypeptide encoded by a nucleic acid molecule comprising the nucleic acid SEQ ID NO. 63807, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 63807 or polypeptide SEQ ID NO. 63808, respectively, or a homolog or a fragment thereof, is increased or generated, or if the activity molecular chaperone portein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 43 to 83-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a YFL0190-protein, or if the activity of the polypeptide Yfl019c, preferably represented by SEQ ID NO. 64145, or a homolog or fragment thereof, or if the activity of a polypeptide encoded by a nucleic acid molecule comprising the nucleic acid SEQ ID NO. 64144, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 64144 or polypeptide SEQ ID NO. 64145, respectively, or a homolog or a fragment thereof, is increased or generated, or if the activity YFL019C-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 32 to 235-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a yfl054c-protein, or if the activity of the polypeptide Yfl054c, preferably represented by SEQ ID NO. 64149, or a homolog or fragment thereof, or if the activity of a polypeptide encoded by a nucleic acid molecule comprising the nucleic acid SEQ ID NO. 64148, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 64148 or polypeptide SEQ ID NO. 64149, respectively, or a homolog or a fragment thereof, is increased or generated, or if the activity yf1054c-protein is increased or generated in a non-human organism, like a microorganism or a plant cell, plant or part thereof, especially with plastidic localization, whereby the respective line discloses in column 7 arginine. For example, an increase of the arginine of at least 1 percent, particularly in a range of 87 to 268-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a yfl054c-protein, or if the activity of the polypeptide Yfl054c, preferably represented by SEQ ID NO. 64149, or a homolog or fragment thereof, or if the activity of a polypeptide encoded by a nucleic acid molecule comprising the nucleic acid SEQ ID NO. 64148, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 64148 or polypeptide SEQ ID NO. 64149, respectively, or a homolog or a fragment thereof, is increased or generated, or if the activity yfl054c-protein is increased or generated in a non-human organism, like a microorganism or a plant cell, plant or part thereof, especially with plastidic localization, whereby the respective line discloses in column 7 glutamine. For example, an increase of the glutamine of at least 1 percent, particularly in a range of 30 to 75-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a pre-mRNA-splicing factor, or if the activity of the polypeptide Ygl174w, preferably represented by SEQ ID NO. 64158, or a homolog or fragment thereof, or if the activity of a polypeptide encoded by a nucleic acid molecule comprising the nucleic acid SEQ ID NO. 64157, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 64157 or polypeptide SEQ ID NO. 64158, respectively, or a homolog or a fragment thereof, is increased or generated, or if the activity pre-mRNA-splicing factor is increased or generated in a non-human organism, like a microorganism or a plant cell, plant or part thereof, especially with plastidic localization, whereby the respective line discloses in column 7 arginine. For example, an increase of the arginine of at least 1 percent, particularly in a range of 85 to 199-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a yg1237c-protein, or if the activity of the polypeptide Yg1237c, preferably represented by SEQ ID NO. 64178, or a homolog or fragment thereof, or if the activity of a polypeptide encoded by a nucleic acid molecule comprising the nucleic acid SEQ ID NO. 64177, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 64177 or polypeptide SEQ ID NO. 64178, respectively, or a homolog or a fragment thereof, is increased or generated, or if the activity ygl237c-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 42 to 388-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a ygr068c-protein, or if the activity of the polypeptide Ygr068c, preferably represented by SEQ ID NO. 64199, or a homolog or fragment thereof, or if the activity of a polypeptide encoded by a nucleic acid molecule comprising the nucleic acid SEQ ID NO. 64198, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 64198 or polypeptide SEQ ID NO. 64199, respectively, or a homolog or a fragment thereof, is increased or generated, or if the activity ygr068c-protein is increased or generated in a non-human organism, like a microorganism or a plant cell, plant or part thereof, especially with plastidic localization, whereby the respective line discloses in column 7 glutamine. For example, an increase of the glutamine of at least 1 percent, particularly in a range of 60 to 428-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a squalene monooxygenase, or if the activity of the polypeptide Ygr175c, preferably represented by SEQ ID NO. 64219, or a homolog or fragment thereof, or if the activity of a polypeptide encoded by a nucleic acid molecule comprising the nucleic acid SEQ ID NO. 64218, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 64218 or polypeptide SEQ ID NO. 64219, respectively, or a homolog or a fragment thereof, is increased or generated, or if the activity squalene monooxygenase is increased or generated in a non-human organism, like a microorganism or a plant cell, plant or part thereof, especially with plastidic localization, whereby the respective line discloses in column 7 proline. For example, an increase of the proline of at least 1 percent, particularly in a range of 43 to 69-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a ygr221c-protein, or if the activity of the polypeptide Ygr221c, preferably represented by SEQ ID NO. 64316, or a homolog or fragment thereof, or if the activity of a polypeptide encoded by a nucleic acid molecule comprising the nucleic acid SEQ ID NO. 64315, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 64315 or polypeptide SEQ ID NO. 64316, respectively, or a homolog or a fragment thereof, is increased or generated, or if the activity ygr221c-protein is increased or generated in a non-human organism, like a microorganism or a plant cell, plant or part thereof, especially with plastidic localization, whereby the respective line discloses in column 7 glutamate. For example, an increase of the glutamate of at least 1 percent, particularly in a range of 33 to 65-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a ygr221c-protein, or if the activity of the polypeptide Ygr221c, preferably represented by SEQ ID NO. 64316, or a homolog or fragment thereof, or if the activity of a polypeptide encoded by a nucleic acid molecule comprising the nucleic acid SEQ ID NO. 64315, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 64315 or polypeptide SEQ ID NO. 64316, respectively, or a homolog or a fragment thereof, is increased or generated, or if the activity ygr221c-protein is increased or generated in a non-human organism, like a microorganism or a plant cell, plant or part thereof, especially with plastidic localization, whereby the respective line discloses in column 7 glutamine. For example, an increase of the glutamine of at least 1 percent, particularly in a range of 24 to 30-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a yhl013c-protein, or if the activity of the polypeptide Yhl013c, preferably represented by SEQ ID NO. 14716, or a homolog or fragment thereof, or if the activity of a polypeptide encoded by a nucleic acid molecule comprising the nucleic acid SEQ ID NO. 14715, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 14715 or polypeptide SEQ ID NO. 14716, respectively, or a homolog or a fragment thereof, is increased or generated, or if the activity yhl013c-protein is increased or generated in a non-human organism, like a microorganism or a plant cell, plant or part thereof, especially with plastidic localization, whereby the respective line discloses in column 7 proline. For example, an increase of the proline of at least 1 percent, particularly in a range of 33 to 402-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a mitochondrial processing protease, or if the activity of the polypeptide Yhr024c, preferably represented by SEQ ID NO. 64337, or a homolog or fragment thereof, or if the activity of a polypeptide encoded by a nucleic acid molecule comprising the nucleic acid SEQ ID NO. 64336, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 64336 or polypeptide SEQ ID NO. 64337, respectively, or a homolog or a fragment thereof, is increased or generated, or if the activity mitochondrial processing protease is increased or generated in a non-human organism, like a microorganism or a plant cell, plant or part thereof, especially with plastidic localization, whereby the respective line discloses in column 7 arginine. For example, an increase of the arginine of at least 1 percent, particularly in a range of 72 to 166-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a H/ACA ribonucleoprotein complex subunit 3, or if the activity of the polypeptide Yhr072w-a, preferably represented by SEQ ID NO. 64471, or a homolog or fragment thereof, or if the activity of a polypeptide encoded by a nucleic acid molecule comprising the nucleic acid SEQ ID NO. 64470, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 64470 or polypeptide SEQ ID NO. 64471, respectively, or a homolog or a fragment thereof, is increased or generated, or if the activity H/ACA ribonucleoprotein complex subunit 3 is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 53 to 260-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a yhr207c-protein, or if the activity of the polypeptide Yhr207c, preferably represented by SEQ ID NO. 64547, or a homolog or fragment thereof, or if the activity of a polypeptide encoded by a nucleic acid molecule comprising the nucleic acid SEQ ID NO. 64546, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 64546 or polypeptide SEQ ID NO. 64547, respectively, or a homolog or a fragment thereof, is increased or generated, or if the activity yhr207c-protein is increased or generated in a non-human organism, like a microorganism or a plant cell, plant or part thereof, especially with plastidic localization, whereby the respective line discloses in column 7 glutamate. For example, an increase of the glutamate of at least 1 percent, particularly in a range of 33 to 47-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a 3-phosphoglycerate dehydrogenase, or if the activity of the polypeptide Yil074c, preferably represented by SEQ ID NO. 64564, or a homolog or fragment thereof, or if the activity of a polypeptide encoded by a nucleic acid molecule comprising the nucleic acid SEQ ID NO. 64563, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 64563 or polypeptide SEQ ID NO. 64564, respectively, or a homolog or a fragment thereof, is increased or generated, or if the activity 3-phosphoglycerate dehydrogenase is increased or generated in a non-human organism, like a microorganism or a plant cell, plant or part thereof, especially with plastidic localization, whereby the respective line discloses in column 7 arginine. For example, an increase of the arginine of at least 1 percent, particularly in a range of 47 to 129-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a allantoinase, or if the activity of the polypeptide Yir027c, preferably represented by SEQ ID NO. 64774, or a homolog or fragment thereof, or if the activity of a polypeptide encoded by a nucleic acid molecule comprising the nucleic acid SEQ ID NO. 64773, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 64773 or polypeptide SEQ ID NO. 64774, respectively, or a homolog or a fragment thereof, is increased or generated, or if the activity allantoinase is increased or generated in a non-human organism, like a microorganism or a plant cell, plant or part thereof, especially with plastidic localization, whereby the respective line discloses in column 7 proline. For example, an increase of the proline of at least 1 percent, particularly in a range of 39 to 46-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a allantoicase, or if the activity of the polypeptide Yir029w, preferably represented by SEQ ID NO. 64895, or a homolog or fragment thereof, or if the activity of a polypeptide encoded by a nucleic acid molecule comprising the nucleic acid SEQ ID NO. 64894, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 64894 or polypeptide SEQ ID NO. 64895, respectively, or a homolog or a fragment thereof, is increased or generated, or if the activity allantoicase is increased or generated in a non-human organism, like a microorganism or a plant cell, plant or part thereof, especially with plastidic localization, whereby the respective line discloses in column 7 arginine. For example, an increase of the arginine of at least 1 percent, particularly in a range of 52 to 316-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a DnaJ-like chaperone, or if the activity of the polypeptide Yj1073w, preferably represented by SEQ ID NO. 64965, or a homolog or fragment thereof, or if the activity of a polypeptide encoded by a nucleic acid molecule comprising the nucleic acid SEQ ID NO. 64964, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 64964 or polypeptide SEQ ID NO. 64965, respectively, or a homolog or a fragment thereof, is increased or generated, or if the activity DnaJ-like chaperone is increased or generated in a non-human organism, like a microorganism or a plant cell, plant or part thereof, especially with plastidic localization, whereby the respective line discloses in column 7 glutamine. For example, an increase of the glutamine of at least 1 percent, particularly in a range of 28 to 29-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a ornithine carbamoyltransferase, or if the activity of the polypeptide Yjl088w, preferably represented by SEQ ID NO. 64976, or a homolog or fragment thereof, or if the activity of a polypeptide encoded by a nucleic acid molecule comprising the nucleic acid SEQ ID NO. 64975, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 64975 or polypeptide SEQ ID NO. 64976, respectively, or a homolog or a fragment thereof, is increased or generated, or if the activity ornithine carbamoyltransferase is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 52 to 124-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a potassium transport protein, or if the activity of the polypeptide Yjl129c, preferably represented by SEQ ID NO. 65182, or a homolog or fragment thereof, or if the activity of a polypeptide encoded by a nucleic acid molecule comprising the nucleic acid SEQ ID NO. 65181, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 65181 or polypeptide SEQ ID NO. 65182, respectively, or a homolog or a fragment thereof, is increased or generated, or if the activity potassium transport protein is increased or generated in a non-human organism, like a microorganism or a plant cell, plant or part thereof, especially with plastidic localization, whereby the respective line discloses in column 7 proline. For example, an increase of the proline of at least 1 percent, particularly in a range of 37 to 89-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a glycogenin, or if the activity of the polypeptide Yjl137c, preferably represented by SEQ ID NO. 14822, or a homolog or fragment thereof, or if the activity of a polypeptide encoded by a nucleic acid molecule comprising the nucleic acid SEQ ID NO. 14821, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 14821 or polypeptide SEQ ID NO. 14822, respectively, or a homolog or a fragment thereof, is increased or generated, or if the activity glycogenin is increased or generated in a non-human organism, like a microorganism or a plant cell, plant or part thereof, especially with plastidic localization, whereby the respective line discloses in column 7 arginine. For example, an increase of the arginine of at least 1 percent, particularly in a range of 47 to 152-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a ATP-dependent RNA helicase, or if the activity of the polypeptide Yjl138c, preferably represented by SEQ ID NO. 65225, or a homolog or fragment thereof, or if the activity of a polypeptide encoded by a nucleic acid molecule comprising the nucleic acid SEQ ID NO. 65224, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 65224 or polypeptide SEQ ID NO. 65225, respectively, or a homolog or a fragment thereof, is increased or generated, or if the activity ATP-dependent RNA helicase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 112 to 770-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a phosphoribosyltransferase, or if the activity of the polypeptide Yjr133w, preferably represented by SEQ ID NO. 66226, or a homolog or fragment thereof, or if the activity of a polypeptide encoded by a nucleic acid molecule comprising the nucleic acid SEQ ID NO. 66225, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 66225 or polypeptide SEQ ID NO. 66226, respectively, or a homolog or a fragment thereof, is increased or generated, or if the activity phosphoribosyltransferase is increased or generated in a non-human organism, like a microorganism or a plant cell, plant or part thereof, especially with plastidic localization, whereby the respective line discloses in column 7 glutamine. For example, an increase of the glutamine of at least 1 percent, particularly in a range of 29 to 46-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a polygalacturonase, or if the activity of the polypeptide Yjr153w, preferably represented by SEQ ID NO. 66275, or a homolog or fragment thereof, or if the activity of a polypeptide encoded by a nucleic acid molecule comprising the nucleic acid SEQ ID NO. 66274, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 66274 or polypeptide SEQ ID NO. 66275, respectively, or a homolog or a fragment thereof, is increased or generated, or if the activity polygalacturonase is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 36 to 124-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a aspartate aminotransferase, or if the activity of the polypeptide Ykl106w, preferably represented by SEQ ID NO. 66420, or a homolog or fragment thereof, or if the activity of a polypeptide encoded by a nucleic acid molecule comprising the nucleic acid SEQ ID NO. 66419, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 66419 or polypeptide SEQ ID NO. 66420, respectively, or a homolog or a fragment thereof, is increased or generated, or if the activity aspartate aminotransferase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 42 to 54-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a ylr065c-protein, or if the activity of the polypeptide Ylr065c, preferably represented by SEQ ID NO. 66696, or a homolog or fragment thereof, or if the activity of a polypeptide encoded by a nucleic acid molecule comprising the nucleic acid SEQ ID NO. 66695, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 66695 or polypeptide SEQ ID NO. 66696, respectively, or a homolog or a fragment thereof, is increased or generated, or if the activity ylr065c-protein is increased or generated in a non-human organism, like a microorganism or a plant cell, plant or part thereof, especially with plastidic localization, whereby the respective line discloses in column 7 glutamate. For example, an increase of the glutamate of at least 1 percent, particularly in a range of 39 to 62-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a ylr178c-protein, or if the activity of the polypeptide Ylr178c, preferably represented by SEQ ID NO. 66716, or a homolog or fragment thereof, or if the activity of a polypeptide encoded by a nucleic acid molecule comprising the nucleic acid SEQ ID NO. 66715, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 66715 or polypeptide SEQ ID NO. 66716, respectively, or a homolog or a fragment thereof, is increased or generated, or if the activity ylr178c-protein is increased or generated in a non-human organism, like a microorganism or a plant cell, plant or part thereof, especially with plastidic localization, whereby the respective line discloses in column 7 glutamine. For example, an increase of the glutamine of at least 1 percent, particularly in a range of 29 to 59-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a aconitate hydratase, or if the activity of the polypeptide Ylr304c, preferably represented by SEQ ID NO. 66773, or a homolog or fragment thereof, or if the activity of a polypeptide encoded by a nucleic acid molecule comprising the nucleic acid SEQ ID NO. 66772, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 66772 or polypeptide SEQ ID NO. 66773, respectively, or a homolog or a fragment thereof, is increased or generated, or if the activity aconitate hydratase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 42 to 53-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a adenylosuccinate lyase, or if the activity of the polypeptide Ylr359w, preferably represented by SEQ ID NO. 67191, or a homolog or fragment thereof, or if the activity of a polypeptide encoded by a nucleic acid molecule comprising the nucleic acid SEQ ID NO. 67190, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 67190 or polypeptide SEQ ID NO. 67191, respectively, or a homolog or a fragment thereof, is increased or generated, or if the activity adenylosuccinate lyase is increased or generated in a non-human organism, like a microorganism or a plant cell, plant or part thereof, especially with plastidic localization, whereby the respective line discloses in column 7 proline. For example, an increase of the proline of at least 1 percent, particularly in a range of 72 to 309-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a ynl142w-protein, or if the activity of the polypeptide Ynl142w, preferably represented by SEQ ID NO. 67300, or a homolog or fragment thereof, or if the activity of a polypeptide encoded by a nucleic acid molecule comprising the nucleic acid SEQ ID NO. 67299, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 67299 or polypeptide SEQ ID NO. 67300, respectively, or a homolog or a fragment thereof, is increased or generated, or if the activity ynl142w-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 98 to 182-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a sterol O-acyltransferase, or if the activity of the polypeptide Ynr019w, preferably represented by SEQ ID NO. 67647, or a homolog or fragment thereof, or if the activity of a polypeptide encoded by a nucleic acid molecule comprising the nucleic acid SEQ ID NO. 67646, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 67646 or polypeptide SEQ ID NO. 67647, respectively, or a homolog or a fragment thereof, is increased or generated, or if the activity sterol O-acyltransferase is increased or generated in a non-human organism, like a microorganism or a plant cell, plant or part thereof, especially with plastidic localization, whereby the respective line discloses in column 7 proline. For example, an increase of the proline of at least 1 percent, particularly in a range of 59 to 77-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a protein kinase, or if the activity of the polypeptide Yol045w, preferably represented by SEQ ID NO. 67685, or a homolog or fragment thereof, or if the activity of a polypeptide encoded by a nucleic acid molecule comprising the nucleic acid SEQ ID NO. 67684, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 67684 or polypeptide SEQ ID NO. 67685, respectively, or a homolog or a fragment thereof, is increased or generated, or if the activity protein kinase is increased or generated in a non-human organism, like a microorganism or a plant cell, plant or part thereof, especially with plastidic localization, whereby the respective line discloses in column 7 arginine. For example, an increase of the arginine of at least 1 percent, particularly in a range of 49 to 140-percent is conferred as compared to a corresponding non-transformed wild type non-human organism.

Accordingly, in one embodiment, an increase of glutamate in a non-human organism, as compared to a corresponding non-transformed wild type non-human organism, is conferred in the process of the invention, if the activity of a polypeptide showing the activity of a glutaminyl-tRNA synthetase, or if the activity of the polypeptide Yor168w, preferably represented by SEQ ID NO. 67711, or a homolog or fragment thereof, or if the activity of a polypeptide encoded by a nucleic acid molecule comprising the nucleic acid SEQ ID NO. 67710, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 67710 or polypeptide SEQ ID NO. 67711, respectively, or a homolog or a fragment thereof, is increased or generated, or if the activity glutaminyl-tRNA synthetase is increased or generated in a non-human organism, like a microorganism or a plant cell, plant or part thereof, especially with non-targeted localization, whereby the respective line discloses in column 7 glutamate. For example, an increase of the glutamate of at least 1 percent, particularly in a range of 38 to 112-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a glutaminyl-tRNA synthetase, or if the activity of the polypeptide Yor168w, preferably represented by SEQ ID NO. 67711, or a homolog or fragment thereof, or if the activity of a polypeptide encoded by a nucleic acid molecule comprising the nucleic acid SEQ ID NO. 67710, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 67710 or polypeptide SEQ ID NO. 67711, respectively, or a homolog or a fragment thereof, is increased or generated, or if the activity glutaminyl-tRNA synthetase is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 28 to 112-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a yor221c-protein, or if the activity of the polypeptide Yor221c, preferably represented by SEQ ID NO. 67952, or a homolog or fragment thereof, or if the activity of a polypeptide encoded by a nucleic acid molecule comprising the nucleic acid SEQ ID NO. 67951, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 67951 or polypeptide SEQ ID NO. 67952, respectively, or a homolog or a fragment thereof, is increased or generated, or if the activity yor221c-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 55 to 420-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a protein kinase, or if the activity of the polypeptide Yor233w, preferably represented by SEQ ID NO. 67969, or a homolog or fragment thereof, or if the activity of a polypeptide encoded by a nucleic acid molecule comprising the nucleic acid SEQ ID NO. 67968, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 67968 or polypeptide SEQ ID NO. 67969, respectively, or a homolog or a fragment thereof, is increased or generated, or if the activity protein kinase is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 40 to 167-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a isopentenyl diphosphate isomerase, or if the activity of the polypeptide Ypl117c, preferably represented by SEQ ID NO. 67999, or a homolog or fragment thereof, or if the activity of a polypeptide encoded by a nucleic acid molecule comprising the nucleic acid SEQ ID NO. 67998, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 67998 or polypeptide SEQ ID NO. 67999, respectively, or a homolog or a fragment thereof, is increased or generated, or if the activity isopentenyl diphosphate isomerase is increased or generated in a non-human organism, like a microorganism or a plant cell, plant or part thereof, especially with plastidic localization, whereby the respective line discloses in column 7 arginine. For example, an increase of the arginine of at least 1 percent, particularly in a range of 77 to 341-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a Zm_{—}4842_BE510522-protein, or if the activity of the polypeptide Zm_{—}4842_BE510522, preferably represented by SEQ ID NO. 68414, or a homolog or fragment thereof, or if the activity of a polypeptide encoded by a nucleic acid molecule comprising the nucleic acid SEQ ID NO. 68413, preferably the coding region thereof, or a homolog or fragment thereof, e.g. derived from Zea mays, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 68413 or polypeptide SEQ ID NO. 68414, respectively, or a homolog or a fragment thereof, is increased or generated, or if the activity Zm_{—}4842_BE510522-protein is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 52 to 238-percent is conferred as compared to a corresponding non-transformed wild type non-human organism.

Accordingly, in one embodiment, an increase of glutamine in a non-human organism, as compared to a corresponding non-transformed wild type non-human organism, is conferred in the process of the invention, if the activity of a polypeptide showing the activity of a Zm_{—}4842_BE510522-protein, or if the activity of the polypeptide Zm_{—}4842_BE510522, preferably represented by SEQ ID NO. 68414, or a homolog or fragment thereof, or if the activity of a polypeptide encoded by a nucleic acid molecule comprising the nucleic acid SEQ ID NO. 68413, preferably the coding region thereof, or a homolog or fragment thereof, e.g. derived from Zea mays, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 68413 or polypeptide SEQ ID NO. 68414, respectively, or a homolog or a fragment thereof, is increased or generated, or if the activity Zm_{—}4842_BE510522-protein is increased or generated in a non-human organism, like a microorganism or a plant cell, plant or part thereof, especially with non-targeted localization, whereby the respective line discloses in column 7 glutamine. For example, an increase of the glutamine of at least 1 percent, particularly in a range of 49 to 411-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a Zm_{—}4842_BE510522protein, or if the activity of the polypeptide Zm_{—}4842_BE510522, preferably represented by SEQ ID NO. 68414, or a homolog or fragment thereof, or if the activity of a polypeptide encoded by a nucleic acid molecule comprising the nucleic acid SEQ ID NO. 68413, preferably the coding region thereof, or a homolog or fragment thereof, e.g. derived from Zea mays, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 68413 or polypeptide SEQ ID NO. 68414, respectively, or a homolog or a fragment thereof, is increased or generated, or if the activity Zm_{—}4842_BE510522-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 44 to 103-percent is conferred as compared to a corresponding non-transformed wild type non-human organism.

Accordingly, in one embodiment, an increase of arginine in a non-human organism, as compared to a corresponding non-transformed wild type non-human organism, is conferred in the process of the invention, if the activity of a polypeptide showing the activity of a phosphopantothenoylcysteine decarboxylase, or if the activity of the polypeptide ZM06LC1143, preferably represented by SEQ ID NO. 68133, or a homolog or fragment thereof, or if the activity of a polypeptide encoded by a nucleic acid molecule comprising the nucleic acid SEQ ID NO. 68132, preferably the coding region thereof, or a homolog or fragment thereof, e.g. derived from Zea mays, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 68132 or polypeptide SEQ ID NO. 68133, respectively, or a homolog or a fragment thereof, is increased or generated, or if the activity phosphopantothenoylcysteine decarboxylase is increased or generated in a non-human organism, like a microorganism or a plant cell, plant or part thereof, especially with non-targeted localization, whereby the respective line discloses in column 7 arginine. For example, an increase of the arginine of at least 1 percent, particularly in a range of 49 to 117-percent is conferred as compared to a corresponding non-transformed wild type non-human organism.

Accordingly, in one embodiment, an increase of proline in a non-human organism, as compared to a corresponding non-transformed wild type non-human organism, is conferred in the process of the invention, if the activity of a polypeptide showing the activity of a ZM06LC11975-protein, or if the activity of the polypeptide ZM06LC11975, preferably represented by SEQ ID NO. 68364, or a homolog or fragment thereof, or if the activity of a polypeptide encoded by a nucleic acid molecule comprising the nucleic acid SEQ ID NO. 68363, preferably the coding region thereof, or a homolog or fragment thereof, e.g. derived from Zea mays, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 2, column 5 or 8 in the respective same line as the nucleic acid molecule SEQ ID NO. 68363 or polypeptide SEQ ID NO. 68364, respectively, or a homolog or a fragment thereof, is increased or generated, or if the activity ZM06LC11975-protein is increased or generated in a non-human organism, like a microorganism or a plant cell, plant or part thereof, especially with non-targeted localization, whereby the respective line discloses in column 7 proline. For example, an increase of the proline of at least 1 percent, particularly in a range of 33 to 136-percent is conferred as compared to a corresponding non-transformed wild type non-human organism.

[0096.1.1.2] to [0103.1.1.2] for the disclosure of these paragraphs see [0096.1.1.1] to [0103.1.1.1] above.

The nucleic acid sequence of 47266012_SOYBEAN from Glycine max, e.g. as shown in the respective line in column 5 of Table I, application no. 2 is unpublished. And the activity of the gene product thereof is the activity of 47266012-protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “47266012-protein”, especially from Glycine max or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said 47266012_SOYBEAN, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said 47266012_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said 47266012_SOYBEAN, or a functional equivalent or a homolog thereof as depicted in column 8 of Table II, application no. 2, preferably a homolog or functional equivalent as depicted in column 8 of Table II B, application no. 2, and being depicted in the same respective line as said 47266012_SOYBEAN, and preferably the activity is increased non-targeted.

Accordingly, in one embodiment, the molecule which activity is to be increased in the process of the invention is the gene product with an activity as a “47266012-protein”, preferably it is the molecule of section (a) or (b) of this paragraph.

In particular, it was observed that in plants, especially in Arabidopsis thaliana, increasing or generating the activity of a gene product non-targeted with the activity of a “47266012-protein”, preferably being encoded by a gene comprising the nucleic acid sequence SEQ ID NO. 15187, preferably the coding region thereof, conferred the production of or the increase in glutamate compared with the wild type control.

The nucleic acid sequence of 47266012_SOYBEAN from Glycine max, e.g. as shown in the respective line in column 5 of Table I, application no. 2 is unpublished. And the activity of the gene product thereof is the activity of 47266012-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “47266012-protein”, especially from Glycine max or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said 47266012_SOYBEAN, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said 47266012_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said 47266012_SOYBEAN, or a functional equivalent or a homolog thereof as depicted in column 8 of Table II, application no. 2, preferably a homolog or functional equivalent as depicted in column 8 of Table II B, application no. 2, and being depicted in the same respective line as said 47266012_SOYBEAN, and preferably the activity is increased non-targeted.

Accordingly, in one embodiment, the molecule which activity is to be increased in the process of the invention is the gene product with an activity as a “47266012-protein”, preferably it is the molecule of section (a) or (b) of this paragraph.

In particular, it was observed that in plants, especially in Arabidopsis thaliana, increasing or generating the activity of a gene product non-targeted with the activity of a “47266012-protein”, preferably being encoded by a gene comprising the nucleic acid sequence SEQ ID NO. 15187, preferably the coding region thereof, conferred the production of or the increase in glutamine compared with the wild type control.

The nucleic acid sequence of 49747384_SOYBEAN from Glycine max, e.g. as shown in the respective line in column 5 of Table I, application no. 2 is unpublished. And the activity of the gene product thereof is the activity of 49747384_SOYBEAN-protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “49747384_SOYBEAN-protein”, especially from Glycine max or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said 49747384_SOYBEAN, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said 49747384_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said 49747384_SOYBEAN, or a functional equivalent or a homolog thereof as depicted in column 8 of Table II, application no. 2, preferably a homolog or functional equivalent as depicted in column 8 of Table II B, application no. 2, and being depicted in the same respective line as said 49747384_SOYBEAN, and preferably the activity is increased non-targeted.

Accordingly, in one embodiment, the molecule which activity is to be increased in the process of the invention is the gene product with an activity as a “49747384_SOYBEAN-protein”, preferably it is the molecule of section (a) or (b) of this paragraph. In particular, it was observed that in plants, especially in Arabidopsis thaliana, increasing or generating the activity of a gene product non-targeted with the activity of a “49747384_SOYBEAN-protein”, preferably being encoded by a gene comprising the nucleic acid sequence SEQ ID NO. 69, preferably the coding region thereof, conferred the production of or the increase in glutamate compared with the wild type control.

The nucleic acid sequence of 51340801_CANOLA from Brassica napus, e.g. as shown in the respective line in column 5 of Table I, application no. 2 is unpublished. And the activity of the gene product thereof is the activity of pyruvate kinase.

Accordingly, in one embodiment, the process of the present invention for producing glutamate in a non-human organism, like a microorganism or a plant or a part thereof, comprises increasing or generating the activity of a gene product with the activity of a gene product conferring the activity of “pyruvate kinase”, especially from Brassica napus or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said 51340801_CANOLA, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said 51340801_CANOLA, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said 51340801_CANOLA, or a functional equivalent or a homolog thereof as depicted in column 8 of Table II, application no. 2, preferably a homolog or functional equivalent as depicted in column 8 of Table II B, application no. 2, and being depicted in the same respective line as said 51340801_CANOLA, and preferably the activity is increased plastidic.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 15532, preferably the coding region thereof, conferred the production of or the increase in glutamate compared with the wild type control.

The nucleic acid sequence of 51340801_CANOLA from Brassica napus, e.g. as shown in the respective line in column 5 of Table I, application no. 2 is unpublished. And the activity of the gene product thereof is the activity of pyruvate kinase.

Accordingly, in one embodiment, the process of the present invention for producing proline in a non-human organism, like a microorganism or a plant or a part thereof, comprises increasing or generating the activity of a gene product with the activity of a gene product conferring the activity of “pyruvate kinase”, especially from Brassica napus or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said 51340801_CANOLA, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said 51340801_CANOLA, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said 51340801_CANOLA, or a functional equivalent or a homolog thereof as depicted in column 8 of Table II, application no. 2, preferably a homolog or functional equivalent as depicted in column 8 of Table II B, application no. 2, and being depicted in the same respective line as said 51340801_CANOLA, and preferably the activity is increased plastidic.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 15532, preferably the coding region thereof, conferred the production of or the increase in proline compared with the wild type control.

The nucleic acid sequence of 59547452_SOYBEAN from Glycine max, e.g. as shown in the respective line in column 5 of Table I, application no. 2 is unpublished. And the activity of the gene product thereof is the activity of acetyltransferase.

Accordingly, in one embodiment, the process of the present invention for producing arginine in a non-human organism, like a microorganism or a plant or a part thereof, comprises increasing or generating the activity of a gene product with the activity of a gene product conferring the activity of “acetyltransferase”, especially from Glycine max or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said 59547452_SOYBEAN, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said 59547452_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said 59547452_SOYBEAN, or a functional equivalent or a homolog thereof as depicted in column 8 of Table II, application no. 2, preferably a homolog or functional equivalent as depicted in column 8 of Table II B, application no. 2, and being depicted in the same respective line as said 59547452_SOYBEAN, and preferably the activity is increased non-targeted.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 16155, preferably the coding region thereof, conferred the production of or the increase in arginine compared with the wild type control.

The nucleic acid sequence of 59554615_SOYBEAN from Glycine max, e.g. as shown in the respective line in column 5 of Table I, application no. 2 is unpublished. And the activity of the gene product thereof is the activity of pyruvate kinase.

Accordingly, in one embodiment, the process of the present invention for producing glutamate in a non-human organism, like a microorganism or a plant or a part thereof, comprises increasing or generating the activity of a gene product with the activity of a gene product conferring the activity of “pyruvate kinase”, especially from Glycine max or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said 59554615_SOYBEAN, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said 59554615_SOYBEAN, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said 59554615_SOYBEAN, or a functional equivalent or a homolog thereof as depicted in column 8 of Table II, application no. 2, preferably a homolog or functional equivalent as depicted in column 8 of Table II B, application no. 2, and being depicted in the same respective line as said 59554615_SOYBEAN, and preferably the activity is increased plastidic.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 16263, preferably the coding region thereof, conferred the production of or the increase in glutamate compared with the wild type control.

The nucleic acid sequence of 59582753_SOYBEAN from Glycine max, e.g. as shown in the respective line in column 5 of Table I, application no. 2 is unpublished. And the activity of the gene product thereof is the activity of glucose-6-phosphate 1-dehydrogenase.

Accordingly, in one embodiment, the process of the present invention for producing arginine in a non-human organism, like a microorganism or a plant or a part thereof, comprises increasing or generating the activity of a gene product with the activity of a gene product conferring the activity of “glucose-6-phosphate 1-dehydrogenase”, especially from Glycine max or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said 59582753_SOYBEAN, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said 59582753_SOYBEAN, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said 59582753_SOYBEAN, or a functional equivalent or a homolog thereof as depicted in column 8 of Table II, application no. 2, preferably a homolog or functional equivalent as depicted in column 8 of Table II B, application no. 2, and being depicted in the same respective line as said 59582753_SOYBEAN, and preferably the activity is increased plastidic.

Accordingly, in one embodiment, the molecule which activity is to be increased in the process of the invention is the gene product with an activity as a “glucose-6-phosphate 1-dehydrogenase”, preferably it is the molecule of section (a) or (b) of this paragraph.

In particular, it was observed that in plants, especially in Arabidopsis thaliana, increasing or generating the activity of a gene product plastidic with the activity of a “glucose-6-phosphate 1-dehydrogenase”, preferably being encoded by a gene comprising the nucleic acid sequence SEQ ID NO. 16883, preferably the coding region thereof, conferred the production of or the increase in arginine compared with the wild type control.

The nucleic acid sequence of 59582753_SOYBEAN from Glycine max, e.g. as shown in the respective line in column 5 of Table I, application no. 2 is unpublished. And the activity of the gene product thereof is the activity of glucose-6-phosphate 1-dehydrogenase.

Accordingly, in one embodiment, the process of the present invention for producing proline in a non-human organism, like a microorganism or a plant or a part thereof, comprises increasing or generating the activity of a gene product with the activity of a gene product conferring the activity of “glucose-6-phosphate 1-dehydrogenase”, especially from Glycine max or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said 59582753_SOYBEAN, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said 59582753_SOYBEAN, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said 59582753_SOYBEAN, or a functional equivalent or a homolog thereof as depicted in column 8 of Table II, application no. 2, preferably a homolog or functional equivalent as depicted in column 8 of Table II B, application no. 2, and being depicted in the same respective line as said 59582753_SOYBEAN, and preferably the activity is increased plastidic.

Accordingly, in one embodiment, the molecule which activity is to be increased in the process of the invention is the gene product with an activity as a “glucose-6-phosphate 1-dehydrogenase”, preferably it is the molecule of section (a) or (b) of this paragraph.

In particular, it was observed that in plants, especially in Arabidopsis thaliana, increasing or generating the activity of a gene product plastidic with the activity of a “glucose-6-phosphate 1-dehydrogenase”, preferably being encoded by a gene comprising the nucleic acid sequence SEQ ID NO. 16883, preferably the coding region thereof, conferred the production of or the increase in proline compared with the wild type control.

CHECK The nucleic acid sequence of AAC43185 from Arabidopsis thaliana, e.g. as shown in the respective line in column 5 of Table I, application no. 2 is unpublished. And the activity of the gene product thereof is the activity of amino acid acetyltransferase.

Accordingly, in one embodiment, the process of the present invention for producing arginine in a non-human organism, like a microorganism or a plant or a part thereof, comprises increasing or generating the activity of a gene product with the activity of a gene product conferring the activity of “amino acid acetyltransferase”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AAC43185, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AAC43185, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AAC43185, or a functional equivalent or a homolog thereof as depicted in column 8 of

Table II, application no. 2, preferably a homolog or functional equivalent as depicted in column 8 of Table II B, application no. 2, and being depicted in the same respective line as said AAC43185, and preferably the activity is increased plastidic.

Accordingly, in one embodiment, the molecule which activity is to be increased in the process of the invention is the gene product with an activity as a “amino acid acetyltransferase”, preferably it is the molecule of section (a) or (b) of this paragraph.

In particular, it was observed that in plants, especially in Arabidopsis thaliana, increasing or generating the activity of a gene product plastidic with the activity of a “amino acid acetyltransferase”, preferably being encoded by a gene comprising the nucleic acid sequence SEQ ID NO. 17356, preferably the coding region thereof, conferred the production of or the increase in arginine compared with the wild type control.

The nucleic acid sequence of At1g07430 from Arabidopsis thaliana, e.g. as shown in the respective line in column 5 of Table I, application no. 2 is unpublished. And the activity of the gene product thereof is the activity of protein phosphatase.

Accordingly, in one embodiment, the process of the present invention for producing glutamate in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At1g07430, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At1g07430, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At1g07430, or a functional equivalent or a homolog thereof as depicted in column 8 of Table II, application no. 2, preferably a homolog or functional equivalent as depicted in column 8 of Table II B, application no. 2, and being depicted in the same respective line as said At1g07430, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 17451, preferably the coding region thereof, conferred the production of or the increase in glutamate compared with the wild type control.

The nucleic acid sequence of At1g07430 from Arabidopsis thaliana, e.g. as shown in the respective line in column 5 of Table I, application no. 2 is unpublished. And the activity of the gene product thereof is the activity of protein phosphatase.

Accordingly, in one embodiment, the process of the present invention for producing glutamine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At1g07430, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At1g07430, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At1g07430, or a functional equivalent or a homolog thereof as depicted in column 8 of Table II, application no. 2, preferably a homolog or functional equivalent as depicted in column 8 of Table II B, application no. 2, and being depicted in the same respective line as said At1g07430, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 17451, preferably the coding region thereof, conferred the production of or the increase in glutamine compared with the wild type control.

The nucleic acid sequence of At1g17440 from Arabidopsis thaliana, e.g. as shown in the respective line in column 5 of Table I, application no. 2 is unpublished. And the activity of the gene product thereof is the activity of At1g17440-protein.

Accordingly, in one embodiment, the process of the present invention for producing arginine in a non-human organism, like a microorganism or a plant or a part thereof, comprises increasing or generating the activity of a gene product with the activity of a gene product conferring the activity of “At1g17440-protein”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At1g17440, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At1g17440, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At1g17440, or a functional equivalent or a homolog thereof as depicted in column 8 of Table II, application no. 2, preferably a homolog or functional equivalent as depicted in column 8 of Table II B, application no. 2, and being depicted in the same respective line as said At1g17440, and preferably the activity is increased non-targeted.

Accordingly, in one embodiment, the molecule which activity is to be increased in the process of the invention is the gene product with an activity as a “At1g17440-protein”, preferably it is the molecule of section (a) or (b) of this paragraph.

In particular, it was observed that in plants, especially in Arabidopsis thaliana, increasing or generating the activity of a gene product non-targeted with the activity of a “At1g17440-protein”, preferably being encoded by a gene comprising the nucleic acid sequence SEQ ID NO. 17601, preferably the coding region thereof, conferred the production of or the increase in arginine compared with the wild type control.

The nucleic acid sequence of At1g19800 from Arabidopsis thaliana, e.g. as shown in the respective line in column 5 of Table I, application no. 2 is unpublished. And the activity of the gene product thereof is the activity of At1g19800-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “At1g19800-protein”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At1g19800, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At1g19800, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At1g19800, or a functional equivalent or a homolog thereof as depicted in column 8 of Table II, application no. 2, preferably a homolog or functional equivalent as depicted in column 8 of Table II B, application no. 2, and being depicted in the same respective line as said At1g19800, and preferably the activity is increased non-targeted.

Accordingly, in one embodiment, the molecule which activity is to be increased in the process of the invention is the gene product with an activity as a “At1g19800-protein”, preferably it is the molecule of section (a) or (b) of this paragraph.

In particular, it was observed that in plants, especially in Arabidopsis thaliana, increasing or generating the activity of a gene product non-targeted with the activity of a “At1g19800-protein”, preferably being encoded by a gene comprising the nucleic acid sequence SEQ ID NO. 17637, preferably the coding region thereof, conferred the production of or the increase in glutamine compared with the wild type control.

The nucleic acid sequence of At1g26830 from Arabidopsis thaliana, e.g. as shown in the respective line in column 5 of Table I, application no. 2 is unpublished. And the activity of the gene product thereof is the activity of cullin.

Accordingly, in one embodiment, the process of the present invention for producing arginine in a non-human organism, like a microorganism or a plant or a part thereof, comprises increasing or generating the activity of a gene product with the activity of a gene product conferring the activity of “cullin”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At1g26830, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At1g26830, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At1g26830, or a functional equivalent or a homolog thereof as depicted in column 8 of Table II, application no. 2, preferably a homolog or functional equivalent as depicted in column 8 of Table II B, application no. 2, and being depicted in the same respective line as said At1g26830, and preferably the activity is increased non-targeted.

Accordingly, in one embodiment, the molecule which activity is to be increased in the process of the invention is the gene product with an activity as a “cullin”, preferably it is the molecule of section (a) or (b) of this paragraph.

In particular, it was observed that in plants, especially in Arabidopsis thaliana, increasing or generating the activity of a gene product non-targeted with the activity of a “cullin”, preferably being encoded by a gene comprising the nucleic acid sequence SEQ ID NO. 17701, preferably the coding region thereof, conferred the production of or the increase in arginine compared with the wild type control.

The nucleic acid sequence of At1g26830 from Arabidopsis thaliana, e.g. as shown in the respective line in column 5 of Table I, application no. 2 is unpublished. And the activity of the gene product thereof is the activity of cullin.

Accordingly, in one embodiment, the process of the present invention for producing proline in a non-human organism, like a microorganism or a plant or a part thereof, comprises increasing or generating the activity of a gene product with the activity of a gene product conferring the activity of “cullin”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At1g26830, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At1g26830, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At1g26830, or a functional equivalent or a homolog thereof as depicted in column 8 of Table II, application no. 2, preferably a homolog or functional equivalent as depicted in column 8 of Table II B, application no. 2, and being depicted in the same respective line as said At1g26830, and preferably the activity is increased non-targeted.

Accordingly, in one embodiment, the molecule which activity is to be increased in the process of the invention is the gene product with an activity as a “cullin”, preferably it is the molecule of section (a) or (b) of this paragraph.

In particular, it was observed that in plants, especially in Arabidopsis thaliana, increasing or generating the activity of a gene product non-targeted with the activity of a “cullin”, preferably being encoded by a gene comprising the nucleic acid sequence SEQ ID NO. 17701, preferably the coding region thereof, conferred the production of or the increase in proline compared with the wild type control.

The nucleic acid sequence of At1g29350 from Arabidopsis thaliana, e.g. as shown in the respective line in column 5 of Table I, application no. 2 is unpublished. And the activity of the gene product thereof is the activity of At1g29350-protein.

Accordingly, in one embodiment, the process of the present invention for producing arginine in a non-human organism, like a microorganism or a plant or a part thereof, comprises increasing or generating the activity of a gene product with the activity of a gene product conferring the activity of “Atl g29350-protein”, especiallyfrom Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At1g29350, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At1g29350, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said

At1g29350, or a functional equivalent or a homolog thereof as depicted in column 8 of Table II, application no. 2, preferably a homolog or functional equivalent as depicted in column 8 of Table II B, application no. 2, and being depicted in the same respective line as said At1g29350, and preferably the activity is increased non-targeted.

Accordingly, in one embodiment, the molecule which activity is to be increased in the process of the invention is the gene product with an activity as a “At1g29350-protein”, preferably it is the molecule of section (a) or (b) of this paragraph.

In particular, it was observed that in plants, especially in Arabidopsis thaliana, increasing or generating the activity of a gene product non-targeted with the activity of a “At1g29350-protein”, preferably being encoded by a gene comprising the nucleic acid sequence SEQ ID NO. 17901, preferably the coding region thereof, conferred the production of or the increase in arginine compared with the wild type control.

The nucleic acid sequence of At1g36730 from Arabidopsis thaliana, e.g. as shown in the respective line in column 5 of Table I, application no. 2 is unpublished. And the activity of the gene product thereof is the activity of eukaryotic translation initiation factor 5.

Accordingly, in one embodiment, the process of the present invention for producing arginine in a non-human organism, like a microorganism or a plant or a part thereof, comprises increasing or generating the activity of a gene product with the activity of a gene product conferring the activity of “eukaryotic translation initiation factor 5”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At1g36730, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At1g36730, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At1g36730, or a functional equivalent or a homolog thereof as depicted in column 8 of Table II, application no. 2, preferably a homolog or functional equivalent as depicted in column 8 of Table II B, application no. 2, and being depicted in the same respective line as said At1g36730, and preferably the activity is increased non-targeted.

Accordingly, in one embodiment, the molecule which activity is to be increased in the process of the invention is the gene product with an activity as a “eukaryotic translation initiation factor 5”, preferably it is the molecule of section (a) or (b) of this paragraph.

In particular, it was observed that in plants, especially in Arabidopsis thaliana, increasing or generating the activity of a gene product non-targeted with the activity of a “eukaryotic translation initiation factor 5”, preferably being encoded by a gene comprising the nucleic acid sequence SEQ ID NO. 17968, preferably the coding region thereof, conferred the production of or the increase in arginine compared with the wild type control.

The nucleic acid sequence of At1g36730 from Arabidopsis thaliana, e.g. as shown in the respective line in column 5 of Table I, application no. 2 is unpublished. And the activity of the gene product thereof is the activity of eukaryotic translation initiation factor 5.

Accordingly, in one embodiment, the process of the present invention for producing glutamine in a non-human organism, like a microorganism or a plant or a part thereof, comprises increasing or generating the activity of a gene product with the activity of a gene product conferring the activity of “eukaryotic translation initiation factor 5”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At1g36730, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At1g36730, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At1g36730, or a functional equivalent or a homolog thereof as depicted in column 8 of Table II, application no. 2, preferably a homolog or functional equivalent as depicted in column 8 of Table II B, application no. 2, and being depicted in the same respective line as said At1g36730, and preferably the activity is increased non-targeted.

Accordingly, in one embodiment, the molecule which activity is to be increased in the process of the invention is the gene product with an activity as a “eukaryotic translation initiation factor 5”, preferably it is the molecule of section (a) or (b) of this paragraph.

In particular, it was observed that in plants, especially in Arabidopsis thaliana, increasing or generating the activity of a gene product non-targeted with the activity of a “eukaryotic translation initiation factor 5”, preferably being encoded by a gene comprising the nucleic acid sequence SEQ ID NO. 17968, preferably the coding region thereof, conferred the production of or the increase in glutamine compared with the wild type control.

The nucleic acid sequence of At1g43850 from Arabidopsis thaliana, e.g. as shown in the respective line in column 5 of Table I, application no. 2 is unpublished. And the activity of the gene product thereof is the activity of transcriptional regulator.

Accordingly, in one embodiment, the process of the present invention for producing arginine in a non-human organism, like a microorganism or a plant or a part thereof, comprises increasing or generating the activity of a gene product with the activity of a gene product conferring the activity of “transcriptional regulator”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At1g43850, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At1g43850, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At1g43850, or a functional equivalent or a homolog thereof as depicted in column 8 of Table II, application no. 2, preferably a homolog or functional equivalent as depicted in column 8 of Table II B, application no. 2, and being depicted in the same respective line as said At1g43850, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph. In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 18070, preferably the coding region thereof, conferred the production of or the increase in arginine compared with the wild type control.

The nucleic acid sequence of At1g43850 from Arabidopsis thaliana, e.g. as shown in the respective line in column 5 of Table I, application no. 2 is unpublished. And the activity of the gene product thereof is the activity of transcriptional regulator.

Accordingly, in one embodiment, the process of the present invention for producing glutamate in a non-human organism, like a microorganism or a plant or a part thereof, comprises increasing or generating the activity of a gene product with the activity of a gene product conferring the activity of “transcriptional regulator”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At1g43850, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At1g43850, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At1g43850, or a functional equivalent or a homolog thereof as depicted in column 8 of Table II, application no. 2, preferably a homolog or functional equivalent as depicted in column 8 of Table II B, application no. 2, and being depicted in the same respective line as said At1g43850, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 18070, preferably the coding region thereof, conferred the production of or the increase in glutamate compared with the wild type control.

The nucleic acid sequence of At1g43850 from Arabidopsis thaliana, e.g. as shown in the respective line in column 5 of Table I, application no. 2 is unpublished. And the activity of the gene product thereof is the activity of transcriptional regulator.

Accordingly, in one embodiment, the process of the present invention for producing glutamine in a non-human organism, like a microorganism or a plant or a part thereof, comprises increasing or generating the activity of a gene product with the activity of a gene product conferring the activity of “transcriptional regulator”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At1g43850, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At1g43850, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At1g43850, or a functional equivalent or a homolog thereof as depicted in column 8 of Table II, application no. 2, preferably a homolog or functional equivalent as depicted in column 8 of Table II B, application no. 2, and being depicted in the same respective line as said At1g43850, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 18070, preferably the coding region thereof, conferred the production of or the increase in glutamine compared with the wild type control.

The nucleic acid sequence of At1g43850 from Arabidopsis thaliana, e.g. as shown in the respective line in column 5 of Table I, application no. 2 is unpublished. And the activity of the gene product thereof is the activity of transcriptional regulator.

Accordingly, in one embodiment, the process of the present invention for producing proline in a non-human organism, like a microorganism or a plant or a part thereof, comprises increasing or generating the activity of a gene product with the activity of a gene product conferring the activity of “transcriptional regulator”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At1g43850, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At1g43850, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At1g43850, or a functional equivalent or a homolog thereof as depicted in column 8 of Table II, application no. 2, preferably a homolog or functional equivalent as depicted in column 8 of Table II B, application no. 2, and being depicted in the same respective line as said At1g43850, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 18070, preferably the coding region thereof, conferred the production of or the increase in proline compared with the wild type control.

The nucleic acid sequence of At1g47380 from Arabidopsis thaliana, e.g. as shown in the respective line in column 5 of Table I, application no. 2 is unpublished. And the activity of the gene product thereof is the activity of At1g47380-protein.

Accordingly, in one embodiment, the process of the present invention for producing proline in a non-human organism, like a microorganism or a plant or a part thereof, comprises increasing or generating the activity of a gene product with the activity of a gene product conferring the activity of “At1g47380-protein”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At1g47380, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At1g47380, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At1g47380, or a functional equivalent or a homolog thereof as depicted in column 8 of Table II, application no. 2, preferably a homolog or functional equivalent as depicted in column 8 of Table II B, application no. 2, and being depicted in the same respective line as said At1g47380, and preferably the activity is increased non-targeted.

Accordingly, in one embodiment, the molecule which activity is to be increased in the process of the invention is the gene product with an activity as a “At1g47380-protein”, preferably it is the molecule of section (a) or (b) of this paragraph.

In particular, it was observed that in plants, especially in Arabidopsis thaliana, increasing or generating the activity of a gene product non-targeted with the activity of a “At1g47380-protein”, preferably being encoded by a gene comprising the nucleic acid sequence SEQ ID NO. 18122, preferably the coding region thereof, conferred the production of or the increase in proline compared with the wild type control.

The nucleic acid sequence of At1g48260 from Arabidopsis thaliana, e.g. as shown in the respective line in column 5 of Table I, application no. 2 is unpublished. And the activity of the gene product thereof is the activity of protein kinase.

Accordingly, in one embodiment, the process of the present invention for producing arginine in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At1g48260, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At1g48260, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At1g48260, or a functional equivalent or a homolog thereof as depicted in column 8 of Table II, application no. 2, preferably a homolog or functional equivalent as depicted in column 8 of Table II B, application no. 2, and being depicted in the same respective line as said At1g48260, and preferably the activity is increased non-targeted.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 18235, preferably the coding region thereof, conferred the production of or the increase in arginine compared with the wild type control.

The nucleic acid sequence of At1g48260 from Arabidopsis thaliana, e.g. as shown in the respective line in column 5 of Table I, application no. 2 is unpublished. And the activity of the gene product thereof is the activity of protein kinase.

Accordingly, in one embodiment, the process of the present invention for producing glutamate in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At1g48260, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At1g48260, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At1g48260, or a functional equivalent or a homolog thereof as depicted in column 8 of Table II, application no. 2, preferably a homolog or functional equivalent as depicted in column 8 of Table II B, application no. 2, and being depicted in the same respective line as said At1g48260, and preferably the activity is increased non-targeted.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 18235, preferably the coding region thereof, conferred the production of or the increase in glutamate compared with the wild type control.

The nucleic acid sequence of At1g48260 from Arabidopsis thaliana, e.g. as shown in the respective line in column 5 of Table I, application no. 2 is unpublished. And the activity of the gene product thereof is the activity of protein kinase.

Accordingly, in one embodiment, the process of the present invention for producing proline in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At1g48260, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At1g48260, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At1g48260, or a functional equivalent or a homolog thereof as depicted in column 8 of Table II, application no. 2, preferably a homolog or functional equivalent as depicted in column 8 of Table II B, application no. 2, and being depicted in the same respective line as said At1g48260, and preferably the activity is increased non-targeted.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 18235, preferably the coding region thereof, conferred the production of or the increase in proline compared with the wild type control.

The nucleic acid sequence of At1g61950 from Arabidopsis thaliana, e.g. as shown in the respective line in column 5 of Table I, application no. 2 is unpublished. And the activity of the gene product thereof is the activity of calcium-dependent protein kinase.

Accordingly, in one embodiment, the process of the present invention for producing arginine in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At1g61950, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At1g61950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At1g61950, or a functional equivalent or a homolog thereof as depicted in column 8 of Table II, application no. 2, preferably a homolog or functional equivalent as depicted in column 8 of Table II B, application no. 2, and being depicted in the same respective line as said At1g61950, and preferably the activity is increased non-targeted.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 18869, preferably the coding region thereof, conferred the production of or the increase in arginine compared with the wild type control.

The nucleic acid sequence of At1g61950 from Arabidopsis thaliana, e.g. as shown in the respective line in column 5 of Table I, application no. 2 is unpublished. And the activity of the gene product thereof is the activity of calcium-dependent protein kinase.

Accordingly, in one embodiment, the process of the present invention for producing glutamine in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At1g61950, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At1g61950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At1g61950, or a functional equivalent or a homolog thereof as depicted in column 8 of Table II, application no. 2, preferably a homolog or functional equivalent as depicted in column 8 of Table II B, application no. 2, and being depicted in the same respective line as said At1g61950, and preferably the activity is increased non-targeted.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 18869, preferably the coding region thereof, conferred the production of or the increase in glutamine compared with the wild type control.

The nucleic acid sequence of At1g67340 from Arabidopsis thaliana, e.g. as shown in the respective line in column 5 of Table I, application no. 2 is unpublished. And the activity of the gene product thereof is the activity of At1g67340-protein.

Accordingly, in one embodiment, the process of the present invention for producing proline in a non-human organism, like a microorganism or a plant or a part thereof, comprises increasing or generating the activity of a gene product with the activity of a gene product conferring the activity of “At1g67340-protein”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At1g67340, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At1g67340, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At1g67340, or a functional equivalent or a homolog thereof as depicted in column 8 of Table II, application no. 2, preferably a homolog or functional equivalent as depicted in column 8 of Table II B, application no. 2, and being depicted in the same respective line as said At1g67340, and preferably the activity is increased non-targeted.

Accordingly, in one embodiment, the molecule which activity is to be increased in the process of the invention is the gene product with an activity as a “At1g67340-protein”, preferably it is the molecule of section (a) or (b) of this paragraph.

In particular, it was observed that in plants, especially in Arabidopsis thaliana, increasing or generating the activity of a gene product non-targeted with the activity of a “At1g67340-protein”, preferably being encoded by a gene comprising the nucleic acid sequence SEQ ID NO. 19364, preferably the coding region thereof, conferred the production of or the increase in proline compared with the wild type control.

The nucleic acid sequence of At1g68320 from Arabidopsis thaliana, e.g. as shown in the respective line in column 5 of Table I, application no. 2 is unpublished. And the activity of the gene product thereof is the activity of transcription factor.

Accordingly, in one embodiment, the process of the present invention for producing glutamine in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At1g68320, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At1g68320, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At1g68320, or a functional equivalent or a homolog thereof as depicted in column 8 of Table II, application no. 2, preferably a homolog or functional equivalent as depicted in column 8 of Table II B, application no. 2, and being depicted in the same respective line as said At1g68320, and preferably the activity is increased non-targeted,.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 1061, preferably the coding region thereof, conferred the production of or the increase in glutamine compared with the wild type control.

The nucleic acid sequence of At1g72770 from Arabidopsis thaliana, e.g. as shown in the respective line in column 5 of Table I, application no. 2 is unpublished. And the activity of the gene product thereof is the activity of protein phosphatase.

Accordingly, in one embodiment, the process of the present invention for producing glutamine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At1g72770, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At1g72770, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At1g72770, or a functional equivalent or a homolog thereof as depicted in column 8 of Table II, application no. 2, preferably a homolog or functional equivalent as depicted in column 8 of Table II B, application no. 2, and being depicted in the same respective line as said At1g72770, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19419, preferably the coding region thereof, conferred the production of or the increase in glutamine compared with the wild type control.

The nucleic acid sequence of At2g17560 from Arabidopsis thaliana, e.g. as shown in the respective line in column 5 of Table I, application no. 2 is unpublished. And the activity of the gene product thereof is the activity of transcription factor.

Accordingly, in one embodiment, the process of the present invention for producing proline in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At2g17560, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At2g17560, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At2g17560, or a functional equivalent or a homolog thereof as depicted in column 8 of Table II, application no. 2, preferably a homolog or functional equivalent as depicted in column 8 of Table II B, application no. 2, and being depicted in the same respective line as said At2g17560, and preferably the activity is increased non-targeted.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 19502, preferably the coding region thereof, conferred the production of or the increase in proline compared with the wild type control.

The nucleic acid sequence of At2g25070 from Arabidopsis thaliana, e.g. as shown in the respective line in column 5 of Table I, application no. 2 is unpublished. And the activity of the gene product thereof is the activity of protein phosphatase.

Accordingly, in one embodiment, the process of the present invention for producing glutamate in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At2g25070, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At2g25070, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At2g25070, or a functional equivalent or a homolog thereof as depicted in column 8 of Table II, application no. 2, preferably a homolog or functional equivalent as depicted in column 8 of Table II B, application no. 2, and being depicted in the same respective line as said At2g25070, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 1298, preferably the coding region thereof, conferred the production of or the increase in glutamate compared with the wild type control.

The nucleic acid sequence of At2g25070 from Arabidopsis thaliana, e.g. as shown in the respective line in column 5 of Table I, application no. 2 is unpublished. And the activity of the gene product thereof is the activity of protein phosphatase.

Accordingly, in one embodiment, the process of the present invention for producing glutamine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At2g25070, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At2g25070, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At2g25070, or a functional equivalent or a homolog thereof as depicted in column 8 of Table II, application no. 2, preferably a homolog or functional equivalent as depicted in column 8 of Table II B, application no. 2, and being depicted in the same respective line as said At2g25070, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 1298, preferably the coding region thereof, conferred the production of or the increase in glutamine compared with the wild type control.

The nucleic acid sequence of At2g26390 from Arabidopsis thaliana, e.g. as shown in the respective line in column 5 of Table I, application no. 2 is unpublished. And the activity of the gene product thereof is the activity of serine protease inhibitor.

Accordingly, in one embodiment, the process of the present invention for producing proline in a non-human organism, like a microorganism or a plant or a part thereof, comprises increasing or generating the activity of a gene product with the activity of a gene product conferring the activity of “serine protease inhibitor”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At2g26390, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At2g26390, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At2g26390, or a functional equivalent or a homolog thereof as depicted in column 8 of Table II, application no. 2, preferably a homolog or functional equivalent as depicted in column 8 of Table II B, application no. 2, and being depicted in the same respective line as said At2g26390, and preferably the activity is increased non-targeted.

Accordingly, in one embodiment, the molecule which activity is to be increased in the process of the invention is the gene product with an activity as a “serine protease inhibitor”, preferably it is the molecule of section (a) or (b) of this paragraph.

In particular, it was observed that in plants, especially in Arabidopsis thaliana, increasing or generating the activity of a gene product non-targeted with the activity of a “serine protease inhibitor”, preferably being encoded by a gene comprising the nucleic acid sequence SEQ ID NO. 19671, preferably the coding region thereof, conferred the production of or the increase in proline compared with the wild type control.

The nucleic acid sequence of At2g28890 from Arabidopsis thaliana, e.g. as shown in the respective line in column 5 of Table I, application no. 2 is unpublished. And the activity of the gene product thereof is the activity of protein phosphatase.

Accordingly, in one embodiment, the process of the present invention for producing proline in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At2g28890, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At2g28890, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At2g28890, or a functional equivalent or a homolog thereof as depicted in column 8 of Table II, application no. 2, preferably a homolog or functional equivalent as depicted in column 8 of Table II B, application no. 2, and being depicted in the same respective line as said At2g28890, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19874, preferably the coding region thereof, conferred the production of or the increase in proline compared with the wild type control.

The nucleic acid sequence of At2g30360 from Arabidopsis thaliana, e.g. as shown in the respective line in column 5 of Table I, application no. 2 is unpublished. And the activity of the gene product thereof is the activity of CBL-interacting protein kinase.

Accordingly, in one embodiment, the process of the present invention for producing glutamine in a non-human organism, like a microorganism or a plant or a part thereof, comprises increasing or generating the activity of a gene product with the activity of a gene product conferring the activity of “CBL-interacting protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At2g30360, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At2g30360, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At2g30360, or a functional equivalent or a homolog thereof as depicted in column 8 of Table II, application no. 2, preferably a homolog or functional equivalent as depicted in column 8 of Table II B, application no. 2, and being depicted in the same respective line as said At2g30360, and preferably the activity is increased non-targeted.

Accordingly, in one embodiment, the molecule which activity is to be increased in the process of the invention is the gene product with an activity as a “CBL-interacting protein kinase”, preferably it is the molecule of section (a) or (b) of this paragraph.

In particular, it was observed that in plants, especially in Arabidopsis thaliana, increasing or generating the activity of a gene product non-targeted with the activity of a “CBL-interacting protein kinase”, preferably being encoded by a gene comprising the nucleic acid sequence SEQ ID NO. 19919, preferably the coding region thereof, conferred the production of or the increase in glutamine compared with the wild type control.

The nucleic acid sequence of At2g30360 from Arabidopsis thaliana, e.g. as shown in the respective line in column 5 of Table I, application no. 2 is unpublished. And the activity of the gene product thereof is the activity of CBL-interacting protein kinase.

Accordingly, in one embodiment, the process of the present invention for producing proline in a non-human organism, like a microorganism or a plant or a part thereof, comprises increasing or generating the activity of a gene product with the activity of a gene product conferring the activity of “CBL-interacting protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At2g30360, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At2g30360, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At2g30360, or a functional equivalent or a homolog thereof as depicted in column 8 of Table II, application no. 2, preferably a homolog or functional equivalent as depicted in column 8 of Table II B, application no. 2, and being depicted in the same respective line as said At2g30360, and preferably the activity is increased non-targeted.

Accordingly, in one embodiment, the molecule which activity is to be increased in the process of the invention is the gene product with an activity as a “CBL-interacting protein kinase”, preferably it is the molecule of section (a) or (b) of this paragraph.

In particular, it was observed that in plants, especially in Arabidopsis thaliana, increasing or generating the activity of a gene product non-targeted with the activity of a “CBL-interacting protein kinase”, preferably being encoded by a gene comprising the nucleic acid sequence SEQ ID NO. 19919, preferably the coding region thereof, conferred the production of or the increase in proline compared with the wild type control.

The nucleic acid sequence of At2g30540 from Arabidopsis thaliana, e.g. as shown in the respective line in column 5 of Table I, application no. 2 is unpublished. And the activity of the gene product thereof is the activity of glutaredoxin.

Accordingly, in one embodiment, the process of the present invention for producing arginine in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At2g30540, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At2g30540, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At2g30540, or a functional equivalent or a homolog thereof as depicted in column 8 of Table II, application no. 2, preferably a homolog or functional equivalent as depicted in column 8 of Table II B, application no. 2, and being depicted in the same respective line as said At2g30540, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 20346, preferably the coding region thereof, conferred the production of or the increase in arginine compared with the wild type control.

The nucleic acid sequence of At2g30540 from Arabidopsis thaliana, e.g. as shown in the respective line in column 5 of Table I, application no. 2 is unpublished. And the activity of the gene product thereof is the activity of glutaredoxin.

Accordingly, in one embodiment, the process of the present invention for producing proline in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At2g30540, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At2g30540, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At2g30540, or a functional equivalent or a homolog thereof as depicted in column 8 of Table II, application no. 2, preferably a homolog or functional equivalent as depicted in column 8 of Table II B, application no. 2, and being depicted in the same respective line as said At2g30540, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 20346, preferably the coding region thereof, conferred the production of or the increase in proline compared with the wild type control.

The nucleic acid sequence of At2g34180 from Arabidopsis thaliana, e.g. as shown in the respective line in column 5 of Table I, application no. 2 is unpublished. And the activity of the gene product thereof is the activity of CBL-interacting protein kinase.

Accordingly, in one embodiment, the process of the present invention for producing proline in a non-human organism, like a microorganism or a plant or a part thereof, comprises increasing or generating the activity of a gene product with the activity of a gene product conferring the activity of “CBL-interacting protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At2g34180, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At2g34180, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At2g34180, or a functional equivalent or a homolog thereof as depicted in column 8 of Table II, application no. 2, preferably a homolog or functional equivalent as depicted in column 8 of Table II B, application no. 2, and being depicted in the same respective line as said At2g34180, and preferably the activity is increased non-targeted.

Accordingly, in one embodiment, the molecule which activity is to be increased in the process of the invention is the gene product with an activity as a “CBL-interacting protein kinase”, preferably it is the molecule of section (a) or (b) of this paragraph.

In particular, it was observed that in plants, especially in Arabidopsis thaliana, increasing or generating the activity of a gene product non-targeted with the activity of a “CBL-interacting protein kinase”, preferably being encoded by a gene comprising the nucleic acid sequence SEQ ID NO. 20578, preferably the coding region thereof, conferred the production of or the increase in proline compared with the wild type control.

The nucleic acid sequence of At2g39800 from Arabidopsis thaliana, e.g. as shown in the respective line in column 5 of Table I, application no. 2 is unpublished. And the activity of the gene product thereof is the activity of delta-1-pyrroline 5-carboxylase synthetase.

Accordingly, in one embodiment, the process of the present invention for producing proline in a non-human organism, like a microorganism or a plant or a part thereof, comprises increasing or generating the activity of a gene product with the activity of a gene product conferring the activity of “delta-1-pyrroline 5-carboxylase synthetase”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At2g39800, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At2g39800, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At2g39800, or a functional equivalent or a homolog thereof as depicted in column 8 of Table II, application no. 2, preferably a homolog or functional equivalent as depicted in column 8 of Table II B, application no. 2, and being depicted in the same respective line as said At2g39800, and preferably the activity is increased non-targeted.

Accordingly, in one embodiment, the molecule which activity is to be increased in the process of the invention is the gene product with an activity as a “delta-1-pyrroline 5-carboxylase synthetase”, preferably it is the molecule of section (a) or (b) of this paragraph.

In particular, it was observed that in plants, especially in Arabidopsis thaliana, increasing or generating the activity of a gene product non-targeted with the activity of a “delta-1-pyrroline 5-carboxylase synthetase”, preferably being encoded by a gene comprising the nucleic acid sequence SEQ ID NO. 21008, preferably the coding region thereof, conferred the production of or the increase in proline compared with the wild type control.

The nucleic acid sequence of At2g46500 from Arabidopsis thaliana, e.g. as shown in the respective line in column 5 of Table I, application no. 2 is unpublished. And the activity of the gene product thereof is the activity of phosphatidylinositol 3- and 4-kinase family protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “phosphatidylinositol 3- and 4-kinase family protein”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At2g46500, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At2g46500, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At2g46500, or a functional equivalent or a homolog thereof as depicted in column 8 of Table II, application no. 2, preferably a homolog or functional equivalent as depicted in column 8 of Table II B, application no. 2, and being depicted in the same respective line as said At2g46500, and preferably the activity is increased non-targeted.

Accordingly, in one embodiment, the molecule which activity is to be increased in the process of the invention is the gene product with an activity as a “phosphatidylinositol 3- and 4-kinase family protein”, preferably it is the molecule of section (a) or (b) of this paragraph.

In particular, it was observed that in plants, especially in Arabidopsis thaliana, increasing or generating the activity of a gene product non-targeted with the activity of a “phosphatidylinositol 3- and 4-kinase family protein”, preferably being encoded by a gene comprising the nucleic acid sequence SEQ ID NO. 21106, preferably the coding region thereof, conferred the production of or the increase in glutamine compared with the wild type control.

The nucleic acid sequence of At2g47880 from Arabidopsis thaliana, e.g. as shown in the respective line in column 5 of Table I, application no. 2 is unpublished. And the activity of the gene product thereof is the activity of glutaredoxin.

Accordingly, in one embodiment, the process of the present invention for producing arginine in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At2g47880, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At2g47880, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At2g47880, or a functional equivalent or a homolog thereof as depicted in column 8 of Table II, application no. 2, preferably a homolog or functional equivalent as depicted in column 8 of Table II B, application no. 2, and being depicted in the same respective line as said At2g47880, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 21159, preferably the coding region thereof, conferred the production of or the increase in arginine compared with the wild type control.

The nucleic acid sequence of At2g47880 from Arabidopsis thaliana, e.g. as shown in the respective line in column 5 of Table I, application no. 2 is unpublished. And the activity of the gene product thereof is the activity of glutaredoxin.

Accordingly, in one embodiment, the process of the present invention for producing glutamine in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At2g47880, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At2g47880, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At2g47880, or a functional equivalent or a homolog thereof as depicted in column 8 of Table II, application no. 2, preferably a homolog or functional equivalent as depicted in column 8 of Table II B, application no. 2, and being depicted in the same respective line as said At2g47880, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 21159, preferably the coding region thereof, conferred the production of or the increase in glutamine compared with the wild type control.

The nucleic acid sequence of At2g47880 from Arabidopsis thaliana, e.g. as shown in the respective line in column 5 of Table I, application no. 2 is unpublished. And the activity of the gene product thereof is the activity of glutaredoxin.

Accordingly, in one embodiment, the process of the present invention for producing proline in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At2g47880, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At2g47880, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At2g47880, or a functional equivalent or a homolog thereof as depicted in column 8 of Table II, application no. 2, preferably a homolog or functional equivalent as depicted in column 8 of Table II B, application no. 2, and being depicted in the same respective line as said At2g47880, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 21159, preferably the coding region thereof, conferred the production of or the increase in proline compared with the wild type control.

The nucleic acid sequence of At3g04050 from Arabidopsis thaliana, e.g. as shown in the respective line in column 5 of Table I, application no. 2 is unpublished. And the activity of the gene product thereof is the activity of pyruvate kinase.

Accordingly, in one embodiment, the process of the present invention for producing glutamate in a non-human organism, like a microorganism or a plant or a part thereof, comprises increasing or generating the activity of a gene product with the activity of a gene product conferring the activity of “pyruvate kinase”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At3g04050, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At3g04050, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At3g04050, or a functional equivalent or a homolog thereof as depicted in column 8 of Table II, application no. 2, preferably a homolog or functional equivalent as depicted in column 8 of Table II B, application no. 2, and being depicted in the same respective line as said At3g04050, and preferably the activity is increased plastidic.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 21497, preferably the coding region thereof, conferred the production of or the increase in glutamate compared with the wild type control.

The nucleic acid sequence of At3g04050 from Arabidopsis thaliana, e.g. as shown in the respective line in column 5 of Table I, application no. 2 is unpublished. And the activity of the gene product thereof is the activity of pyruvate kinase.

Accordingly, in one embodiment, the process of the present invention for producing proline in a non-human organism, like a microorganism or a plant or a part thereof, comprises increasing or generating the activity of a gene product with the activity of a gene product conferring the activity of “pyruvate kinase”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At3g04050, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At3g04050, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At3g04050, or a functional equivalent or a homolog thereof as depicted in column 8 of Table II, application no. 2, preferably a homolog or functional equivalent as depicted in column 8 of Table II B, application no. 2, and being depicted in the same respective line as said At3g04050, and preferably the activity is increased plastidic.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 21497, preferably the coding region thereof, conferred the production of or the increase in proline compared with the wild type control.

The nucleic acid sequence of At3g04710 from Arabidopsis thaliana, e.g. as shown in the respective line in column 5 of Table I, application no. 2 is unpublished. And the activity of the gene product thereof is the activity of ankyrin repeat family protein.

Accordingly, in one embodiment, the process of the present invention for producing proline in a non-human organism, like a microorganism or a plant or a part thereof, comprises increasing or generating the activity of a gene product with the activity of a gene product conferring the activity of “ankyrin repeat family protein”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At3g04710, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At3g04710, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At3g04710, or a functional equivalent or a homolog thereof as depicted in column 8 of Table II, application no. 2, preferably a homolog or functional equivalent as depicted in column 8 of Table II B, application no. 2, and being depicted in the same respective line as said At3g04710, and preferably the activity is increased non-targeted.

Accordingly, in one embodiment, the molecule which activity is to be increased in the process of the invention is the gene product with an activity as a “ankyrin repeat family protein”, preferably it is the molecule of section (a) or (b) of this paragraph.

In particular, it was observed that in plants, especially in Arabidopsis thaliana, increasing or generating the activity of a gene product non-targeted with the activity of a “ankyrin repeat family protein”, preferably being encoded by a gene comprising the nucleic acid sequence SEQ ID NO. 21902, preferably the coding region thereof, conferred the production of or the increase in proline compared with the wild type control.

The nucleic acid sequence of At3g06270 from Arabidopsis thaliana, e.g. as shown in the respective line in column 5 of Table I, application no. 2 is unpublished. And the activity of the gene product thereof is the activity of protein phosphatase.

Accordingly, in one embodiment, the process of the present invention for producing glutamate in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At3g06270, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At3g06270, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At3g06270, or a functional equivalent or a homolog thereof as depicted in column 8 of Table II, application no. 2, preferably a homolog or functional equivalent as depicted in column 8 of Table II B, application no. 2, and being depicted in the same respective line as said At3g06270, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 22015, preferably the coding region thereof, conferred the production of or the increase in glutamate compared with the wild type control.

The nucleic acid sequence of At3g08710 from Arabidopsis thaliana, e.g. as shown in the respective line in column 5 of Table I, application no. 2 is unpublished. And the activity of the gene product thereof is the activity of thioredoxin family protein.

Accordingly, in one embodiment, the process of the present invention for producing arginine in a non-human organism, like a microorganism or a plant or a part thereof, comprises increasing or generating the activity of a gene product with the activity of a gene product conferring the activity of “thioredoxin family protein”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At3g08710, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At3g08710, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At3g08710, or a functional equivalent or a homolog thereof as depicted in column 8 of Table II, application no. 2, preferably a homolog or functional equivalent as depicted in column 8 of Table II B, application no. 2, and being depicted in the same respective line as said At3g08710, and preferably the activity is increased non-targeted.

Accordingly, in one embodiment, the molecule which activity is to be increased in the process of the invention is the gene product with an activity as a “thioredoxin family protein”, preferably it is the molecule of section (a) or (b) of this paragraph.

In particular, it was observed that in plants, especially in Arabidopsis thaliana, increasing or generating the activity of a gene product non-targeted with the activity of a “thioredoxin family protein”, preferably being encoded by a gene comprising the nucleic acid sequence SEQ ID NO. 22249, preferably the coding region thereof, conferred the production of or the increase in arginine compared with the wild type control.

The nucleic acid sequence of At3g08710 from Arabidopsis thaliana, e.g. as shown in the respective line in column 5 of Table I, application no. 2 is unpublished. And the activity of the gene product thereof is the activity of thioredoxin family protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “thioredoxin family protein”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At3g08710, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At3g08710, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At3g08710, or a functional equivalent or a homolog thereof as depicted in column 8 of Table II, application no. 2, preferably a homolog or functional equivalent as depicted in column 8 of Table II B, application no. 2, and being depicted in the same respective line as said At3g08710, and preferably the activity is increased non-targeted.

Accordingly, in one embodiment, the molecule which activity is to be increased in the process of the invention is the gene product with an activity as a “thioredoxin family protein”, preferably it is the molecule of section (a) or (b) of this paragraph.

In particular, it was observed that in plants, especially in Arabidopsis thaliana, increasing or generating the activity of a gene product non-targeted with the activity of a “thioredoxin family protein”, preferably being encoded by a gene comprising the nucleic acid sequence SEQ ID NO. 22249, preferably the coding region thereof, conferred the production of or the increase in glutamate compared with the wild type control.

The nucleic acid sequence of At3g08710 from Arabidopsis thaliana, e.g. as shown in the respective line in column 5 of Table I, application no. 2 is unpublished. And the activity of the gene product thereof is the activity of thioredoxin family protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “thioredoxin family protein”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At3g08710, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At3g08710, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At3g08710, or a functional equivalent or a homolog thereof as depicted in column 8 of Table II, application no. 2, preferably a homolog or functional equivalent as depicted in column 8 of Table II B, application no. 2, and being depicted in the same respective line as said At3g08710, and preferably the activity is increased non-targeted.

Accordingly, in one embodiment, the molecule which activity is to be increased in the process of the invention is the gene product with an activity as a “thioredoxin family protein”, preferably it is the molecule of section (a) or (b) of this paragraph.

In particular, it was observed that in plants, especially in Arabidopsis thaliana, increasing or generating the activity of a gene product non-targeted with the activity of a “thioredoxin family protein”, preferably being encoded by a gene comprising the nucleic acid sequence SEQ ID NO. 22249, preferably the coding region thereof, conferred the production of or the increase in glutamine compared with the wild type control.

The nucleic acid sequence of At3g08710 from Arabidopsis thaliana, e.g. as shown in the respective line in column 5 of Table I, application no. 2 is unpublished. And the activity of the gene product thereof is the activity of thioredoxin family protein.

Accordingly, in one embodiment, the process of the present invention for producing proline in a non-human organism, like a microorganism or a plant or a part thereof, comprises increasing or generating the activity of a gene product with the activity of a gene product conferring the activity of “thioredoxin family protein”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At3g08710, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At3g08710, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At3g08710, or a functional equivalent or a homolog thereof as depicted in column 8 of Table II, application no. 2, preferably a homolog or functional equivalent as depicted in column 8 of Table II B, application no. 2, and being depicted in the same respective line as said At3g08710, and preferably the activity is increased non-targeted.

Accordingly, in one embodiment, the molecule which activity is to be increased in the process of the invention is the gene product with an activity as a “thioredoxin family protein”, preferably it is the molecule of section (a) or (b) of this paragraph.

In particular, it was observed that in plants, especially in Arabidopsis thaliana, increasing or generating the activity of a gene product non-targeted with the activity of a “thioredoxin family protein”, preferably being encoded by a gene comprising the nucleic acid sequence SEQ ID NO. 22249, preferably the coding region thereof, conferred the production of or the increase in proline compared with the wild type control.

The nucleic acid sequence of At3g11650 from Arabidopsis thaliana, e.g. as shown in the respective line in column 5 of Table I, application no. 2 is unpublished. And the activity of the gene product thereof is the activity of harpin-induced family protein.

Accordingly, in one embodiment, the process of the present invention for producing arginine in a non-human organism, like a microorganism or a plant or a part thereof, comprises increasing or generating the activity of a gene product with the activity of a gene product conferring the activity of “harpin-induced family protein”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At3g11650, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At3g11650, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At3g11650, or a functional equivalent or a homolog thereof as depicted in column 8 of Table II, application no. 2, preferably a homolog or functional equivalent as depicted in column 8 of Table II B, application no. 2, and being depicted in the same respective line as said At3g11650, and preferably the activity is increased non-targeted,.

Accordingly, in one embodiment, the molecule which activity is to be increased in the process of the invention is the gene product with an activity as a “harpin-induced family protein”, preferably it is the molecule of section (a) or (b) of this paragraph.

In particular, it was observed that in plants, especially in Arabidopsis thaliana, increasing or generating the activity of a gene product non-targeted with the activity of a “harpin-induced family protein”, preferably being encoded by a gene comprising the nucleic acid sequence SEQ ID NO. 22611, preferably the coding region thereof, conferred the production of or the increase in arginine compared with the wild type control.

The nucleic acid sequence of At3g11650 from Arabidopsis thaliana, e.g. as shown in the respective line in column 5 of Table I, application no. 2 is unpublished. And the activity of the gene product thereof is the activity of harpin-induced family protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “harpin-induced family protein”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At3g11650, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At3g11650, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At3g11650, or a functional equivalent or a homolog thereof as depicted in column 8 of Table II, application no. 2, preferably a homolog or functional equivalent as depicted in column 8 of Table II B, application no. 2, and being depicted in the same respective line as said At3g11650, and preferably the activity is increased non-targeted.

Accordingly, in one embodiment, the molecule which activity is to be increased in the process of the invention is the gene product with an activity as a “harpin-induced family protein”, preferably it is the molecule of section (a) or (b) of this paragraph.

In particular, it was observed that in plants, especially in Arabidopsis thaliana, increasing or generating the activity of a gene product non-targeted with the activity of a “harpin-induced family protein”, preferably being encoded by a gene comprising the nucleic acid sequence SEQ ID NO. 22611, preferably the coding region thereof, conferred the production of or the increase in glutamine compared with the wild type control.

The nucleic acid sequence of At3g11650 from Arabidopsis thaliana, e.g. as shown in the respective line in column 5 of Table I, application no. 2 is unpublished. And the activity of the gene product thereof is the activity of harpin-induced family protein.

Accordingly, in one embodiment, the process of the present invention for producing proline in a non-human organism, like a microorganism or a plant or a part thereof, comprises increasing or generating the activity of a gene product with the activity of a gene product conferring the activity of “harpin-induced family protein”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At3g11650, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At3g11650, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At3g11650, or a functional equivalent or a homolog thereof as depicted in column 8 of Table II, application no. 2, preferably a homolog or functional equivalent as depicted in column 8 of Table II B, application no. 2, and being depicted in the same respective line as said At3g11650, and preferably the activity is increased non-targeted.

Accordingly, in one embodiment, the molecule which activity is to be increased in the process of the invention is the gene product with an activity as a “harpin-induced family protein”, preferably it is the molecule of section (a) or (b) of this paragraph.

In particular, it was observed that in plants, especially in Arabidopsis thaliana, increasing or generating the activity of a gene product non-targeted with the activity of a “harpin-induced family protein”, preferably being encoded by a gene comprising the nucleic acid sequence SEQ ID NO. 22611, preferably the coding region thereof, conferred the production of or the increase in proline compared with the wild type control.

The nucleic acid sequence of At3g14230 from Arabidopsis thaliana, e.g. as shown in the respective line in column 5 of Table I, application no. 2 is unpublished. And the activity of the gene product thereof is the activity of DNA binding protein.

Accordingly, in one embodiment, the process of the present invention for producing proline in a non-human organism, like a microorganism or a plant or a part thereof, comprises increasing or generating the activity of a gene product with the activity of a gene product conferring the activity of “DNA binding protein”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At3g14230, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At3g14230, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At3g14230, or a functional equivalent or a homolog thereof as depicted in column 8 of Table II, application no. 2, preferably a homolog or functional equivalent as depicted in column 8 of Table II B, application no. 2, and being depicted in the same respective line as said At3g14230, and preferably the activity is increased plastidic.

Accordingly, in one embodiment, the molecule which activity is to be increased in the process of the invention is the gene product with an activity as a “DNA binding protein”, preferably it is the molecule of section (a) or (b) of this paragraph.

In particular, it was observed that in plants, especially in Arabidopsis thaliana, increasing or generating the activity of a gene product plastidic with the activity of a “DNA binding protein”, preferably being encoded by a gene comprising the nucleic acid sequence SEQ ID NO. 22699, preferably the coding region thereof, conferred the production of or the increase in proline compared with the wild type control.

The nucleic acid sequence of At3g18524 from Arabidopsis thaliana, e.g. as shown in the respective line in column 5 of Table I, application no. 2 is unpublished. And the activity of the gene product thereof is the activity of DNA mismatch repair protein.

Accordingly, in one embodiment, the process of the present invention for producing proline in a non-human organism, like a microorganism or a plant or a part thereof, comprises increasing or generating the activity of a gene product with the activity of a gene product conferring the activity of “DNA mismatch repair protein”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At3g18524, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At3g18524, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At3g18524, or a functional equivalent or a homolog thereof as depicted in column 8 of Table II, application no. 2, preferably a homolog or functional equivalent as depicted in column 8 of Table II B, application no. 2, and being depicted in the same respective line as said At3g18524, and preferably the activity is increased non-targeted.

Accordingly, in one embodiment, the molecule which activity is to be increased in the process of the invention is the gene product with an activity as a “DNA mismatch repair protein”, preferably it is the molecule of section (a) or (b) of this paragraph.

In particular, it was observed that in plants, especially in Arabidopsis thaliana, increasing or generating the activity of a gene product non-targeted with the activity of a “DNA mismatch repair protein”, preferably being encoded by a gene comprising the nucleic acid sequence SEQ ID NO. 22832, preferably the coding region thereof, conferred the production of or the increase in proline compared with the wild type control.

The nucleic acid sequence of At3g20910 from Arabidopsis thaliana, e.g. as shown in the respective line in column 5 of Table I, application no. 2 is unpublished. And the activity of the gene product thereof is the activity of CCAAT-binding transcription factor.

Accordingly, in one embodiment, the process of the present invention for producing proline in a non-human organism, like a microorganism or a plant or a part thereof, comprises increasing or generating the activity of a gene product with the activity of a gene product conferring the activity of “CCAAT-binding transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At3g20910, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At3g20910, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At3g20910, or a functional equivalent or a homolog thereof as depicted in column 8 of Table II, application no. 2, preferably a homolog or functional equivalent as depicted in column 8 of Table II B, application no. 2, and being depicted in the same respective line as said At3g20910, and preferably the activity is increased non-targeted.

Accordingly, in one embodiment, the molecule which activity is to be increased in the process of the invention is the gene product with an activity as a “CCAAT-binding transcription factor”, preferably it is the molecule of section (a) or (b) of this paragraph.

In particular, it was observed that in plants, especially in Arabidopsis thaliana, increasing or generating the activity of a gene product non-targeted with the activity of a “CCAAT-binding transcription factor”, preferably being encoded by a gene comprising the nucleic acid sequence SEQ ID NO. 22921, preferably the coding region thereof, conferred the production of or the increase in proline compared with the wild type control.

The nucleic acid sequence of At3g23000 from Arabidopsis thaliana, e.g. as shown in the respective line in column 5 of Table I, application no. 2 is unpublished. And the activity of the gene product thereof is the activity of protein kinase.

Accordingly, in one embodiment, the process of the present invention for producing glutamine in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At3g23000, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At3g23000, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At3g23000, or a functional equivalent or a homolog thereof as depicted in column 8 of Table II, application no. 2, preferably a homolog or functional equivalent as depicted in column 8 of Table II B, application no. 2, and being depicted in the same respective line as said At3g23000, and preferably the activity is increased non-targeted.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 1815, preferably the coding region thereof, conferred the production of or the increase in glutamine compared with the wild type control.

The nucleic acid sequence of At3g27300 from Arabidopsis thaliana, e.g. as shown in the respective line in column 5 of Table I, application no. 2 is unpublished. And the activity of the gene product thereof is the activity of glucose-6-phosphate 1-dehydrogenase.

Accordingly, in one embodiment, the process of the present invention for producing proline in a non-human organism, like a microorganism or a plant or a part thereof, comprises increasing or generating the activity of a gene product with the activity of a gene product conferring the activity of “glucose-6-phosphate 1-dehydrogenase”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At3g27300, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At3g27300, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At3g27300, or a functional equivalent or a homolog thereof as depicted in column 8 of Table II, application no. 2, preferably a homolog or functional equivalent as depicted in column 8 of Table II B, application no. 2, and being depicted in the same respective line as said At3g27300, and preferably the activity is increased non-targeted.

Accordingly, in one embodiment, the molecule which activity is to be increased in the process of the invention is the gene product with an activity as a “glucose-6-phosphate 1-dehydrogenase”, preferably it is the molecule of section (a) or (b) of this paragraph.

In particular, it was observed that in plants, especially in Arabidopsis thaliana, increasing or generating the activity of a gene product non-targeted with the activity of a “glucose-6-phosphate 1-dehydrogenase”, preferably being encoded by a gene comprising the nucleic acid sequence SEQ ID NO. 23002, preferably the coding region thereof, conferred the production of or the increase in proline compared with the wild type control.

The nucleic acid sequence of At3g62930 from Arabidopsis thaliana, e.g. as shown in the respective line in column 5 of Table I, application no. 2 is unpublished. And the activity of the gene product thereof is the activity of monthiol glutaredoxin.

Accordingly, in one embodiment, the process of the present invention for producing glutamate in a non-human organism, like a microorganism or a plant or a part thereof, comprises increasing or generating the activity of a gene product with the activity of a gene product conferring the activity of “monthiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At3g62930, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At3g62930, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At3g62930, or a functional equivalent or a homolog thereof as depicted in column 8 of Table II, application no. 2, preferably a homolog or functional equivalent as depicted in column 8 of Table II B, application no. 2, and being depicted in the same respective line as said At3g62930, and preferably the activity is increased non-targeted.

Accordingly, in one embodiment, the molecule which activity is to be increased in the process of the invention is the gene product with an activity as a “monthiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monthiol glutaredoxin”, preferably being encoded by a gene comprising the nucleic acid sequence SEQ ID NO. 2573, preferably the coding region thereof, conferred the production of or the increase in glutamate compared with the wild type control.

The nucleic acid sequence of At3g62930 from Arabidopsis thaliana, e.g. as shown in the respective line in column 5 of Table I, application no. 2 is unpublished. And the activity of the gene product thereof is the activity of monthiol glutaredoxin.

Accordingly, in one embodiment, the process of the present invention for producing glutamine in a non-human organism, like a microorganism or a plant or a part thereof, comprises increasing or generating the activity of a gene product with the activity of a gene product conferring the activity of “monthiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At3g62930, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At3g62930, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At3g62930, or a functional equivalent or a homolog thereof as depicted in column 8 of Table II, application no. 2, preferably a homolog or functional equivalent as depicted in column 8 of Table II B, application no. 2, and being depicted in the same respective line as said At3g62930, and preferably the activity is increased non-targeted.

Accordingly, in one embodiment, the molecule which activity is to be increased in the process of the invention is the gene product with an activity as a “monthiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monthiol glutaredoxin”, preferably being encoded by a gene comprising the nucleic acid sequence SEQ ID NO. 2573, preferably the coding region thereof, conferred the production of or the increase in glutamine compared with the wild type control.

The nucleic acid sequence of At3g62950 from Arabidopsis thaliana, e.g. as shown in the respective line in column 5 of Table I, application no. 2 is unpublished. And the activity of the gene product thereof is the activity of glutaredoxin.

Accordingly, in one embodiment, the process of the present invention for producing glutamate in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At3g62950, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At3g62950, or a functional equivalent or a homolog thereof as depicted in column 8 of Table II, application no. 2, preferably a homolog or functional equivalent as depicted in column 8 of Table II B, application no. 2, and being depicted in the same respective line as said At3g62950, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in glutamate compared with the wild type control.

The nucleic acid sequence of At3g62950 from Arabidopsis thaliana, e.g. as shown in the respective line in column 5 of Table I, application no. 2 is unpublished. And the activity of the gene product thereof is the activity of glutaredoxin.

Accordingly, in one embodiment, the process of the present invention for producing glutamine in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At3g62950, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At3g62950, or a functional equivalent or a homolog thereof as depicted in column 8 of Table II, application no. 2, preferably a homolog or functional equivalent as depicted in column 8 of Table II B, application no. 2, and being depicted in the same respective line as said At3g62950, and preferably the activity is increased non-targeted,.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in glutamine compared with the wild type control.

The nucleic acid sequence of At4g15660 from Arabidopsis thaliana, e.g. as shown in the respective line in column 5 of Table I, application no. 2 is unpublished. And the activity of the gene product thereof is the activity of monothiol glutaredoxin.

Accordingly, in one embodiment, the process of the present invention for producing glutamate in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At4g15660, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At4g15660, or a functional equivalent or a homolog thereof as depicted in column 8 of Table II, application no. 2, preferably a homolog or functional equivalent as depicted in column 8 of Table II B, application no. 2, and being depicted in the same respective line as said At4g15660, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in glutamate compared with the wild type control.

The nucleic acid sequence of At4g15660 from Arabidopsis thaliana, e.g. as shown in the respective line in column 5 of Table I, application no. 2 is unpublished. And the activity of the gene product thereof is the activity of monothiol glutaredoxin.

Accordingly, in one embodiment, the process of the present invention for producing glutamine in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At4g15660, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At4g15660, or a functional equivalent or a homolog thereof as depicted in column 8 of Table II, application no. 2, preferably a homolog or functional equivalent as depicted in column 8 of Table II B, application no. 2, and being depicted in the same respective line as said At4g15660, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in glutamine compared with the wild type control.

The nucleic acid sequence of At4g15670 from Arabidopsis thaliana, e.g. as shown in the respective line in column 5 of Table I, application no. 2 is unpublished. And the activity of the gene product thereof is the activity of monothiol glutaredoxin.

Accordingly, in one embodiment, the process of the present invention for producing glutamate in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At4g15670, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At4g15670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At4g15670, or a functional equivalent or a homolog thereof as depicted in column 8 of Table II, application no. 2, preferably a homolog or functional equivalent as depicted in column 8 of Table II B, application no. 2, and being depicted in the same respective line as said At4g15670, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3279, preferably the coding region thereof, conferred the production of or the increase in glutamate compared with the wild type control.

The nucleic acid sequence of At4g15690 from Arabidopsis thaliana, e.g. as shown in the respective line in column 5 of Table I, application no. 2 is unpublished. And the activity of the gene product thereof is the activity of monothiol glutaredoxin.

Accordingly, in one embodiment, the process of the present invention for producing glutamate in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At4g15690, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At4g15690, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At4g15690, or a functional equivalent or a homolog thereof as depicted in column 8 of Table II, application no. 2, preferably a homolog or functional equivalent as depicted in column 8 of Table II B, application no. 2, and being depicted in the same respective line as said At4g15690, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23844, preferably the coding region thereof, conferred the production of or the increase in glutamate compared with the wild type control.

The nucleic acid sequence of At4g15690 from Arabidopsis thaliana, e.g. as shown in the respective line in column 5 of Table I, application no. 2 is unpublished. And the activity of the gene product thereof is the activity of monothiol glutaredoxin.

Accordingly, in one embodiment, the process of the present invention for producing glutamine in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At4g15690, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At4g15690, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At4g15690, or a functional equivalent or a homolog thereof as depicted in column 8 of Table II, application no. 2, preferably a homolog or functional equivalent as depicted in column 8 of Table II B, application no. 2, and being depicted in the same respective line as said At4g15690, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23844, preferably the coding region thereof, conferred the production of or the increase in glutamine compared with the wild type control.

The nucleic acid sequence of At4g15690 from Arabidopsis thaliana, e.g. as shown in the respective line in column 5 of Table I, application no. 2 is unpublished. And the activity of the gene product thereof is the activity of monothiol glutaredoxin.

Accordingly, in one embodiment, the process of the present invention for producing proline in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At4g15690, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At4g15690, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At4g15690, or a functional equivalent or a homolog thereof as depicted in column 8 of Table II, application no. 2, preferably a homolog or functional equivalent as depicted in column 8 of Table II B, application no. 2, and being depicted in the same respective line as said At4g15690, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23844, preferably the coding region thereof, conferred the production of or the increase in proline compared with the wild type control.

The nucleic acid sequence of At4g15700 from Arabidopsis thaliana, e.g. as shown in the respective line in column 5 of Table I, application no. 2 is unpublished. And the activity of the gene product thereof is the activity of monothiol glutaredoxin.

Accordingly, in one embodiment, the process of the present invention for producing glutamine in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At4g15700, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At4g15700, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At4g15700, or a functional equivalent or a homolog thereof as depicted in column 8 of Table II, application no. 2, preferably a homolog or functional equivalent as depicted in column 8 of Table II B, application no. 2, and being depicted in the same respective line as said At4g15700, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3654, preferably the coding region thereof, conferred the production of or the increase in glutamine compared with the wild type control.

The nucleic acid sequence of At4g18880 from Arabidopsis thaliana, e.g. as shown in the respective line in column 5 of Table I, application no. 2 is unpublished. And the activity of the gene product thereof is the activity of heat shock transcription factor.

Accordingly, in one embodiment, the process of the present invention for producing proline in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At4g18880, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At4g18880, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At4g18880, or a functional equivalent or a homolog thereof as depicted in column 8 of Table II, application no. 2, preferably a homolog or functional equivalent as depicted in column 8 of Table II B, application no. 2, and being depicted in the same respective line as said At4g18880, and preferably the activity is increased non-targeted.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 24232, preferably the coding region thereof, conferred the production of or the increase in proline compared with the wild type control.

The nucleic acid sequence of At4g32480 from Arabidopsis thaliana, e.g. as shown in the respective line in column 5 of Table I, application no. 2, is unpublished. And the activity of the gene product thereof is the activity of At4g32480-protein.

Accordingly, in one embodiment, the process of the present invention for producing arginine in a non-human organism, like a microorganism or a plant or a part thereof, comprises increasing or generating the activity of a gene product with the activity of a gene product conferring the activity of “At4g32480-protein”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At4g32480, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At4g32480, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At4g32480, or a functional equivalent or a homolog thereof as depicted in column 8 of Table II, application no. 2, preferably a homolog or functional equivalent as depicted in column 8 of Table II B, application no. 2, and being depicted in the same respective line as said At4g32480, and preferably the activity is increased non-targeted,.

Accordingly, in one embodiment, the molecule which activity is to be increased in the process of the invention is the gene product with an activity as a “At4g32480-protein”, preferably it is the molecule of section (a) or (b) of this paragraph.

In particular, it was observed that in plants, especially in Arabidopsis thaliana, increasing or generating the activity of a gene product non-targeted with the activity of a “At4g32480-protein”, preferably being encoded by a gene comprising the nucleic acid sequence SEQ ID NO. 4040, preferably the coding region thereof, conferred the production of or the increase in arginine compared with the wild type control.

The nucleic acid sequence of At4g32480 from Arabidopsis thaliana, e.g. as shown in the respective line in column 5 of Table I, application no. 2 is unpublished. And the activity of the gene product thereof is the activity of At4g32480-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “At4g32480-protein”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At4g32480, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At4g32480, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At4g32480, or a functional equivalent or a homolog thereof as depicted in column 8 of Table II, application no. 2, preferably a homolog or functional equivalent as depicted in column 8 of Table II B, application no. 2, and being depicted in the same respective line as said At4g32480, and preferably the activity is increased non-targeted.

Accordingly, in one embodiment, the molecule which activity is to be increased in the process of the invention is the gene product with an activity as a “At4g32480-protein”, preferably it is the molecule of section (a) or (b) of this paragraph.

In particular, it was observed that in plants, especially in Arabidopsis thaliana, increasing or generating the activity of a gene product non-targeted with the activity of a “At4g32480-protein”, preferably being encoded by a gene comprising the nucleic acid sequence SEQ ID NO. 4040, preferably the coding region thereof, conferred the production of or the increase in glutamine compared with the wild type control.

The nucleic acid sequence of At4g32480 from Arabidopsis thaliana, e.g. as shown in the respective line in column 5 of Table I, application no. 2 is unpublished. And the activity of the gene product thereof is the activity of At4g32480-protein.

Accordingly, in one embodiment, the process of the present invention for producing proline in a non-human organism, like a microorganism or a plant or a part thereof, comprises increasing or generating the activity of a gene product with the activity of a gene product conferring the activity of “At4g32480-protein”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At4g32480, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At4g32480, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At4g32480, or a functional equivalent or a homolog thereof as depicted in column 8 of Table II, application no. 2, preferably a homolog or functional equivalent as depicted in column 8 of Table II B, application no. 2, and being depicted in the same respective line as said At4g32480, and preferably the activity is increased non-targeted.

Accordingly, in one embodiment, the molecule which activity is to be increased in the process of the invention is the gene product with an activity as a “At4g32480-protein”, preferably it is the molecule of section (a) or (b) of this paragraph.

In particular, it was observed that in plants, especially in Arabidopsis thaliana, increasing or generating the activity of a gene product non-targeted with the activity of a “At4g32480-protein”, preferably being encoded by a gene comprising the nucleic acid sequence SEQ ID NO. 4040, preferably the coding region thereof, conferred the production of or the increase in proline compared with the wild type control.

The nucleic acid sequence of At4g33040 from Arabidopsis thaliana, e.g. as shown in the respective line in column 5 of Table I, application no. 2 is unpublished. And the activity of the gene product thereof is the activity of glutaredoxin.

Accordingly, in one embodiment, the process of the present invention for producing glutamate in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At4g33040, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At4g33040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At4g33040, or a functional equivalent or a homolog thereof as depicted in column 8 of Table II, application no. 2, preferably a homolog or functional equivalent as depicted in column 8 of Table II B, application no. 2, and being depicted in the same respective line as said At4g33040, and preferably the activity is increased non-targeted,.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 4102, preferably the coding region thereof, conferred the production of or the increase in glutamate compared with the wild type control.

The nucleic acid sequence of At4g34160 from Arabidopsis thaliana, e.g. as shown in the respective line in column 5 of Table I, application no. 2 is unpublished. And the activity of the gene product thereof is the activity of cyclin D.

Accordingly, in one embodiment, the process of the present invention for producing arginine in a non-human organism, like a microorganism or a plant or a part thereof, comprises increasing or generating the activity of a gene product with the activity of a gene product conferring the activity of “cyclin D”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At4g34160, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At4g34160, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At4g34160, or a functional equivalent or a homolog thereof as depicted in column 8 of Table II, application no. 2, preferably a homolog or functional equivalent as depicted in column 8 of Table II B, application no. 2, and being depicted in the same respective line as said At4g34160, and preferably the activity is increased non-targeted.

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 24311, preferably the coding region thereof, conferred the production of or the increase in arginine compared with the wild type control.

The nucleic acid sequence of At4g34160 from Arabidopsis thaliana, e.g. as shown in the respective line in column 5 of Table I, application no. 2 is unpublished. And the activity of the gene product thereof is the activity of cyclin D.

Accordingly, in one embodiment, the process of the present invention for producing glutamine in a non-human organism, like a microorganism or a plant or a part thereof, comprises increasing or generating the activity of a gene product with the activity of a gene product conferring the activity of “cyclin D”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At4g34160, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At4g34160, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At4g34160, or a functional equivalent or a homolog thereof as depicted in column 8 of Table II, application no. 2, preferably a homolog or functional equivalent as depicted in column 8 of Table II B, application no. 2, and being depicted in the same respective line as said At4g34160, and preferably the activity is increased non-targeted.

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 24311, preferably the coding region thereof, conferred the production of or the increase in glutamine compared with the wild type control.

The nucleic acid sequence of At4g34160 from Arabidopsis thaliana, e.g. as shown in the respective line in column 5 of Table I, application no. 2 is unpublished. And the activity of the gene product thereof is the activity of cyclin D.

Accordingly, in one embodiment, the process of the present invention for producing proline in a non-human organism, like a microorganism or a plant or a part thereof, comprises increasing or generating the activity of a gene product with the activity of a gene product conferring the activity of “cyclin D”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At4g34160, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At4g34160, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At4g34160, or a functional equivalent or a homolog thereof as depicted in column 8 of Table II, application no. 2, preferably a homolog or functional equivalent as depicted in column 8 of Table II B, application no. 2, and being depicted in the same respective line as said At4g34160, and preferably the activity is increased non-targeted.

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 24311, preferably the coding region thereof, conferred the production of or the increase in proline compared with the wild type control.

The nucleic acid sequence of At4g35310 from Arabidopsis thaliana, e.g. as shown in the respective line in column 5 of Table I, application no. 2 is unpublished. And the activity of the gene product thereof is the activity of calcium-dependent protein kinase.

Accordingly, in one embodiment, the process of the present invention for producing arginine in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At4g35310, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At4g35310, or a functional equivalent or a homolog thereof as depicted in column 8 of Table II, application no. 2, preferably a homolog or functional equivalent as depicted in column 8 of Table II B, application no. 2, and being depicted in the same respective line as said At4g35310, and preferably the activity is increased non-targeted.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in arginine compared with the wild type control.

The nucleic acid sequence of At4g35310 from Arabidopsis thaliana, e.g. as shown in the respective line in column 5 of Table I, application no. 2 is unpublished. And the activity of the gene product thereof is the activity of calcium-dependent protein kinase.

Accordingly, in one embodiment, the process of the present invention for producing proline in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At4g35310, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At4g35310, or a functional equivalent or a homolog thereof as depicted in column 8 of Table II, application no. 2, preferably a homolog or functional equivalent as depicted in column 8 of Table II B, application no. 2, and being depicted in the same respective line as said At4g35310, and preferably the activity is increased non-targeted.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in proline compared with the wild type control.

The nucleic acid sequence of At5g03720 from Arabidopsis thaliana, e.g. as shown in the respective line in column 5 of Table I, application no. 2 is unpublished. And the activity of the gene product thereof is the activity of heat shock transcription factor.

Accordingly, in one embodiment, the process of the present invention for producing glutamate in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At5g03720, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At5g03720, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At5g03720, or a functional equivalent or a homolog thereof as depicted in column 8 of Table II, application no. 2, preferably a homolog or functional equivalent as depicted in column 8 of Table II B, application no. 2, and being depicted in the same respective line as said At5g03720, and preferably the activity is increased non-targeted.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 24438, preferably the coding region thereof, conferred the production of or the increase in glutamate compared with the wild type control.

The nucleic acid sequence of At5g07200 from Arabidopsis thaliana, e.g. as shown in the respective line in column 5 of Table I, application no. 2 is unpublished. And the activity of the gene product thereof is the activity of gibberellin 20-oxidase.

Accordingly, in one embodiment, the process of the present invention for producing proline in a non-human organism, like a microorganism or a plant or a part thereof, comprises increasing or generating the activity of a gene product with the activity of a gene product conferring the activity of “gibberellin 20-oxidase”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At5g07200, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At5g07200, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At5g07200, or a functional equivalent or a homolog thereof as depicted in column 8 of Table II, application no. 2, preferably a homolog or functional equivalent as depicted in column 8 of Table II B, application no. 2, and being depicted in the same respective line as said At5g07200, and preferably the activity is increased non-targeted.

Accordingly, in one embodiment, the molecule which activity is to be increased in the process of the invention is the gene product with an activity as a “gibberellin 20-oxidase”, preferably it is the molecule of section (a) or (b) of this paragraph.

In particular, it was observed that in plants, especially in Arabidopsis thaliana, increasing or generating the activity of a gene product non-targeted with the activity of a “gibberellin 20-oxidase”, preferably being encoded by a gene comprising the nucleic acid sequence SEQ ID NO. 24492, preferably the coding region thereof, conferred the production of or the increase in proline compared with the wild type control.

The nucleic acid sequence of At5g10820 from Arabidopsis thaliana, e.g. as shown in the respective line in column 5 of Table I, application no. 2 is unpublished. And the activity of the gene product thereof is the activity of integral membrane transporter family protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “integral membrane transporter family protein”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At5g10820, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At5g10820, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At5g10820, or a functional equivalent or a homolog thereof as depicted in column 8 of Table II, application no. 2, preferably a homolog or functional equivalent as depicted in column 8 of Table II B, application no. 2, and being depicted in the same respective line as said At5g10820, and preferably the activity is increased non-targeted.

Accordingly, in one embodiment, the molecule which activity is to be increased in the process of the invention is the gene product with an activity as a “integral membrane transporter family protein”, preferably it is the molecule of section (a) or (b) of this paragraph.

In particular, it was observed that in plants, especially in Arabidopsis thaliana, increasing or generating the activity of a gene product non-targeted with the activity of a “integral membrane transporter family protein”, preferably being encoded by a gene comprising the nucleic acid sequence SEQ ID NO. 25222, preferably the coding region thereof, conferred the production of or the increase in glutamate compared with the wild type control.

The nucleic acid sequence of At5g10820 from Arabidopsis thaliana, e.g. as shown in the respective line in column 5 of Table I, application no. 2 is unpublished. And the activity of the gene product thereof is the activity of integral membrane transporter family protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “integral membrane transporter family protein”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At5g10820, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At5g10820, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At5g10820, or a functional equivalent or a homolog thereof as depicted in column 8 of Table II, application no. 2, preferably a homolog or functional equivalent as depicted in column 8 of Table II B, application no. 2, and being depicted in the same respective line as said At5g10820, and preferably the activity is increased non-targeted.

Accordingly, in one embodiment, the molecule which activity is to be increased in the process of the invention is the gene product with an activity as a “integral membrane transporter family protein”, preferably it is the molecule of section (a) or (b) of this paragraph.

In particular, it was observed that in plants, especially in Arabidopsis thaliana, increasing or generating the activity of a gene product non-targeted with the activity of a “integral membrane transporter family protein”, preferably being encoded by a gene comprising the nucleic acid sequence SEQ ID NO. 25222, preferably the coding region thereof, conferred the production of or the increase in glutamine compared with the wild type control.

The nucleic acid sequence of At5g16650 from Arabidopsis thaliana, e.g. as shown in the respective line in column 5 of Table I, application no. 2 is unpublished. And the activity of the gene product thereof is the activity of At5g16650-protein.

Accordingly, in one embodiment, the process of the present invention for producing arginine in a non-human organism, like a microorganism or a plant or a part thereof, comprises increasing or generating the activity of a gene product with the activity of a gene product conferring the activity of “At5g16650-protein”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At5g16650, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At5g16650, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At5g16650, or a functional equivalent or a homolog thereof as depicted in column 8 of Table II, application no. 2, preferably a homolog or functional equivalent as depicted in column 8 of Table II B, application no. 2, and being depicted in the same respective line as said At5g16650, and preferably the activity is increased non-targeted,.

Accordingly, in one embodiment, the molecule which activity is to be increased in the process of the invention is the gene product with an activity as a “At5g16650-protein”, preferably it is the molecule of section (a) or (b) of this paragraph.

In particular, it was observed that in plants, especially in Arabidopsis thaliana, increasing or generating the activity of a gene product non-targeted with the activity of a “At5g16650-protein”, preferably being encoded by a gene comprising the nucleic acid sequence SEQ ID NO. 25283, preferably the coding region thereof, conferred the production of or the increase in arginine compared with the wild type control.

The nucleic acid sequence of At5g16650 from Arabidopsis thaliana, e.g. as shown in the respective line in column 5 of Table I, application no. 2 is unpublished. And the activity of the gene product thereof is the activity of At5g16650-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “At5g16650-protein”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At5g16650, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At5g16650, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At5g16650, or a functional equivalent or a homolog thereof as depicted in column 8 of Table II, application no. 2, preferably a homolog or functional equivalent as depicted in column 8 of Table II B, application no. 2, and being depicted in the same respective line as said At5g16650, and preferably the activity is increased non-targeted.

Accordingly, in one embodiment, the molecule which activity is to be increased in the process of the invention is the gene product with an activity as a “At5g16650-protein”, preferably it is the molecule of section (a) or (b) of this paragraph.

In particular, it was observed that in plants, especially in Arabidopsis thaliana, increasing or generating the activity of a gene product non-targeted with the activity of a “At5g16650-protein”, preferably being encoded by a gene comprising the nucleic acid sequence SEQ ID NO. 25283, preferably the coding region thereof, conferred the production of or the increase in glutamine compared with the wild type control.

The nucleic acid sequence of At5g16650 from Arabidopsis thaliana, e.g. as shown in the respective line in column 5 of Table I, application no. 2 is unpublished. And the activity of the gene product thereof is the activity of At5g16650-protein.

Accordingly, in one embodiment, the process of the present invention for producing proline in a non-human organism, like a microorganism or a plant or a part thereof, comprises increasing or generating the activity of a gene product with the activity of a gene product conferring the activity of “At5g16650-protein”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At5g16650, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At5g16650, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At5g16650, or a functional equivalent or a homolog thereof as depicted in column 8 of Table II, application no. 2, preferably a homolog or functional equivalent as depicted in column 8 of Table II B, application no. 2, and being depicted in the same respective line as said At5g16650, and preferably the activity is increased non-targeted.

Accordingly, in one embodiment, the molecule which activity is to be increased in the process of the invention is the gene product with an activity as a “At5g16650-protein”, preferably it is the molecule of section (a) or (b) of this paragraph.

In particular, it was observed that in plants, especially in Arabidopsis thaliana, increasing or generating the activity of a gene product non-targeted with the activity of a “At5g16650-protein”, preferably being encoded by a gene comprising the nucleic acid sequence SEQ ID NO. 25283, preferably the coding region thereof, conferred the production of or the increase in proline compared with the wild type control.

The nucleic acid sequence of At5g18600 from Arabidopsis thaliana, e.g. as shown in the respective line in column 5 of Table I, application no. 2 is unpublished. And the activity of the gene product thereof is the activity of monothiol glutaredoxin.

Accordingly, in one embodiment, the process of the present invention for producing glutamine in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At5g18600, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At5g18600, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At5g18600, or a functional equivalent or a homolog thereof as depicted in column 8 of Table II, application no. 2, preferably a homolog or functional equivalent as depicted in column 8 of Table II B, application no. 2, and being depicted in the same respective line as said At5g18600, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 4904, preferably the coding region thereof, conferred the production of or the increase in glutamine compared with the wild type control.

The nucleic acid sequence of At5g27640 from Arabidopsis thaliana, e.g. as shown in the respective line in column 5 of Table I, application no. 2 is unpublished. And the activity of the gene product thereof is the activity of translation initiation factor subunit.

Accordingly, in one embodiment, the process of the present invention for producing arginine in a non-human organism, like a microorganism or a plant or a part thereof, comprises increasing or generating the activity of a gene product with the activity of a gene product conferring the activity of “translation initiation factor subunit”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At5g27640, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At5g27640, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At5g27640, or a functional equivalent or a homolog thereof as depicted in column 8 of Table II, application no. 2, preferably a homolog or functional equivalent as depicted in column 8 of Table II B, application no. 2, and being depicted in the same respective line as said At5g27640, and preferably the activity is increased non-targeted.

Accordingly, in one embodiment, the molecule which activity is to be increased in the process of the invention is the gene product with an activity as a “translation initiation factor subunit”, preferably it is the molecule of section (a) or (b) of this paragraph.

In particular, it was observed that in plants, especially in Arabidopsis thaliana, increasing or generating the activity of a gene product non-targeted with the activity of a “translation initiation factor subunit”, preferably being encoded by a gene comprising the nucleic acid sequence SEQ ID NO. 25344, preferably the coding region thereof, conferred the production of or the increase in arginine compared with the wild type control.

The nucleic acid sequence of At5g39760 from Arabidopsis thaliana, e.g. as shown in the respective line in column 5 of Table I, application no. 2 is unpublished. And the activity of the gene product thereof is the activity of transcription factor.

Accordingly, in one embodiment, the process of the present invention for producing glutamate in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At5g39760, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At5g39760, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At5g39760, or a functional equivalent or a homolog thereof as depicted in column 8 of Table II, application no. 2, preferably a homolog or functional equivalent as depicted in column 8 of Table II B, application no. 2, and being depicted in the same respective line as said At5g39760, and preferably the activity is increased non-targeted.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 25428, preferably the coding region thereof, conferred the production of or the increase in glutamate compared with the wild type control.

The nucleic acid sequence of At5g39760 from Arabidopsis thaliana, e.g. as shown in the respective line in column 5 of Table I, application no. 2 is unpublished. And the activity of the gene product thereof is the activity of transcription factor.

Accordingly, in one embodiment, the process of the present invention for producing proline in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At5g39760, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At5g39760, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At5g39760, or a functional equivalent or a homolog thereof as depicted in column 8 of Table II, application no. 2, preferably a homolog or functional equivalent as depicted in column 8 of Table II B, application no. 2, and being depicted in the same respective line as said At5g39760, and preferably the activity is increased non-targeted.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 25428, preferably the coding region thereof, conferred the production of or the increase in proline compared with the wild type control.

The nucleic acid sequence of At5g57050 from Arabidopsis thaliana, e.g. as shown in the respective line in column 5 of Table I, application no. 2 is unpublished. And the activity of the gene product thereof is the activity of protein phosphatase.

Accordingly, in one embodiment, the process of the present invention for producing glutamine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At5g57050, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At5g57050, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At5g57050, or a functional equivalent or a homolog thereof as depicted in column 8 of Table II, application no. 2, preferably a homolog or functional equivalent as depicted in column 8 of Table II B, application no. 2, and being depicted in the same respective line as said At5g57050, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 5318, preferably the coding region thereof, conferred the production of or the increase in glutamine compared with the wild type control.

The nucleic acid sequence of At5g59220 from Arabidopsis thaliana, e.g. as shown in the respective line in column 5 of Table I, application no. 2 is unpublished. And the activity of the gene product thereof is the activity of protein phosphatase.

Accordingly, in one embodiment, the process of the present invention for producing glutamine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At5g59220, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At5g59220, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At5g59220, or a functional equivalent or a homolog thereof as depicted in column 8 of Table II, application no. 2, preferably a homolog or functional equivalent as depicted in column 8 of Table II B, application no. 2, and being depicted in the same respective line as said At5g59220, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 25498, preferably the coding region thereof, conferred the production of or the increase in glutamine compared with the wild type control.

The nucleic acid sequence of At5g64920 from Arabidopsis thaliana, e.g. as shown in the respective line in column 5 of Table I, application no. 2 is unpublished. And the activity of the gene product thereof is the activity of zinc finger protein.

Accordingly, in one embodiment, the process of the present invention for producing arginine in a non-human organism, like a microorganism or a plant or a part thereof, comprises increasing or generating the activity of a gene product with the activity of a gene product conferring the activity of “zinc finger protein”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At5g64920, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At5g64920, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At5g64920, or a functional equivalent or a homolog thereof as depicted in column 8 of Table II, application no. 2, preferably a homolog or functional equivalent as depicted in column 8 of Table II B, application no. 2, and being depicted in the same respective line as said At5g64920, and preferably the activity is increased non-targeted.

Accordingly, in one embodiment, the molecule which activity is to be increased in the process of the invention is the gene product with an activity as a “zinc finger protein”, preferably it is the molecule of section (a) or (b) of this paragraph.

In particular, it was observed that in plants, especially in Arabidopsis thaliana, increasing or generating the activity of a gene product non-targeted with the activity of a “zinc finger protein”, preferably being encoded by a gene comprising the nucleic acid sequence SEQ ID NO. 5493, preferably the coding region thereof, conferred the production of or the increase in arginine compared with the wild type control.

The nucleic acid sequence of At5g64920 from Arabidopsis thaliana, e.g. as shown in the respective line in column 5 of Table I, application no. 2 is unpublished. And the activity of the gene product thereof is the activity of zinc finger protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “zinc finger protein”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said At5g64920, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said At5g64920, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said At5g64920, or a functional equivalent or a homolog thereof as depicted in column 8 of Table II, application no. 2, preferably a homolog or functional equivalent as depicted in column 8 of Table II B, application no. 2, and being depicted in the same respective line as said At5g64920, and preferably the activity is increased non-targeted.

Accordingly, in one embodiment, the molecule which activity is to be increased in the process of the invention is the gene product with an activity as a “zinc finger protein”, preferably it is the molecule of section (a) or (b) of this paragraph.

In particular, it was observed that in plants, especially in Arabidopsis thaliana, increasing or generating the activity of a gene product non-targeted with the activity of a “zinc finger protein”, preferably being encoded by a gene comprising the nucleic acid sequence SEQ ID NO. 5493, preferably the coding region thereof, conferred the production of or the increase in glutamine compared with the wild type control.

The nucleic acid sequence of AvinDRAFT_{—}1045 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of transcriptional regulator.

Accordingly, in one embodiment, the process of the present invention for producing glutamate in a non-human organism, like a microorganism or a plant or a part thereof, comprises increasing or generating the activity of a gene product with the activity of a gene product conferring the activity of “transcriptional regulator”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}1045, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}1045, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}1045, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}1045, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 25676, preferably the coding region thereof, conferred the production of or the increase in glutamate compared with the wild type control.

The nucleic acid sequence of AvinDRAFT_{—}1398 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of adenylylsulfate kinase.

Accordingly, in one embodiment, the process of the present invention for producing glutamate in a non-human organism, like a microorganism or a plant or a part thereof, comprises increasing or generating the activity of a gene product with the activity of a gene product conferring the activity of “adenylylsulfate kinase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}1398, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}1398, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}1398, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}1398, and preferably the activity is increased non-targeted.

Accordingly, in one embodiment, the molecule which activity is to be increased in the process of the invention is the gene product with an activity as a “adenylylsulfate kinase”, preferably it is the molecule of section (a) or (b) of this paragraph.

In particular, it was observed that in plants, especially in Arabidopsis thaliana, increasing or generating the activity of a gene product non-targeted with the activity of a “adenylylsulfate kinase”, preferably being encoded by a gene comprising the nucleic acid sequence SEQ ID NO. 25780, preferably the coding region thereof, conferred the production of or the increase in glutamate compared with the wild type control.

The nucleic acid sequence of AvinDRAFT_{—}1398 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of adenylylsulfate kinase.

Accordingly, in one embodiment, the process of the present invention for producing glutamine in a non-human organism, like a microorganism or a plant or a part thereof, comprises increasing or generating the activity of a gene product with the activity of a gene product conferring the activity of “adenylylsulfate kinase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}1398, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}1398, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}1398, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}1398, and preferably the activity is increased non-targeted.

Accordingly, in one embodiment, the molecule which activity is to be increased in the process of the invention is the gene product with an activity as a “adenylylsulfate kinase”, preferably it is the molecule of section (a) or (b) of this paragraph.

In particular, it was observed that in plants, especially in Arabidopsis thaliana, increasing or generating the activity of a gene product non-targeted with the activity of a “adenylylsulfate kinase”, preferably being encoded by a gene comprising the nucleic acid sequence SEQ ID NO. 25780, preferably the coding region thereof, conferred the production of or the increase in glutamine compared with the wild type control.

The nucleic acid sequence of AvinDRAFT_{—}1495 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of malic enzyme.

Accordingly, in one embodiment, the process of the present invention for producing glutamine in a non-human organism, like a microorganism or a plant or a part thereof, comprises increasing or generating the activity of a gene product with the activity of a gene product conferring the activity of “malic enzyme”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}1495, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}1495, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}1495, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}1495, and preferably the activity is increased non-targeted.

Accordingly, in one embodiment, the molecule which activity is to be increased in the process of the invention is the gene product with an activity as a “malic enzyme”, preferably it is the molecule of section (a) or (b) of this paragraph.

In particular, it was observed that in plants, especially in Arabidopsis thaliana, increasing or generating the activity of a gene product non-targeted with the activity of a “malic enzyme”, preferably being encoded by a gene comprising the nucleic acid sequence SEQ ID NO. 5557, preferably the coding region thereof, conferred the production of or the increase in glutamine compared with the wild type control.

The nucleic acid sequence of AvinDRAFT_{—}1495 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of malic enzyme.

Accordingly, in one embodiment, the process of the present invention for producing proline in a non-human organism, like a microorganism or a plant or a part thereof, comprises increasing or generating the activity of a gene product with the activity of a gene product conferring the activity of “malic enzyme”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}1495, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}1495, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}1495, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}1495, and preferably the activity is increased non-targeted.

Accordingly, in one embodiment, the molecule which activity is to be increased in the process of the invention is the gene product with an activity as a “malic enzyme”, preferably it is the molecule of section (a) or (b) of this paragraph.

In particular, it was observed that in plants, especially in Arabidopsis thaliana, increasing or generating the activity of a gene product non-targeted with the activity of a “malic enzyme”, preferably being encoded by a gene comprising the nucleic acid sequence SEQ ID NO. 5557, preferably the coding region thereof, conferred the production of or the increase in proline compared with the wild type control.

The nucleic acid sequence of AvinDRAFT_{—}1534 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of glycosyl transferase.

Accordingly, in one embodiment, the process of the present invention for producing proline in a non-human organism, like a microorganism or a plant or a part thereof, comprises increasing or generating the activity of a gene product with the activity of a gene product conferring the activity of “glycosyl transferase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}1534, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}1534, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}1534, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}1534, and preferably the activity is increased non-targeted.

Accordingly, in one embodiment, the molecule which activity is to be increased in the process of the invention is the gene product with an activity as a “glycosyl transferase”, preferably it is the molecule of section (a) or (b) of this paragraph.

In particular, it was observed that in plants, especially in Arabidopsis thaliana, increasing or generating the activity of a gene product non-targeted with the activity of a “glycosyl transferase”, preferably being encoded by a gene comprising the nucleic acid sequence SEQ ID NO. 26120, preferably the coding region thereof, conferred the production of or the increase in proline compared with the wild type control.

The nucleic acid sequence of AvinDRAFT_{—}1624 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of sec-independent protein translocase.

Accordingly, in one embodiment, the process of the present invention for producing proline in a non-human organism, like a microorganism or a plant or a part thereof, comprises increasing or generating the activity of a gene product with the activity of a gene product conferring the activity of “sec-independent protein translocase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}1624, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}1624, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}1624, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}1624, and preferably the activity is increased non-targeted.

Accordingly, in one embodiment, the molecule which activity is to be increased in the process of the invention is the gene product with an activity as a “sec-independent protein translocase”, preferably it is the molecule of section (a) or (b) of this paragraph.

In particular, it was observed that in plants, especially in Arabidopsis thaliana, increasing or generating the activity of a gene product non-targeted with the activity of a “sec-independent protein translocase”, preferably being encoded by a gene comprising the nucleic acid sequence SEQ ID NO. 26196, preferably the coding region thereof, conferred the production of or the increase in proline compared with the wild type control.

The nucleic acid sequence of AvinDRAFT_{—}1806 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of pyruvate kinase.

Accordingly, in one embodiment, the process of the present invention for producing glutamate in a non-human organism, like a microorganism or a plant or a part thereof, comprises increasing or generating the activity of a gene product with the activity of a gene product conferring the activity of “pyruvate kinase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}1806, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}1806, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}1806, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}1806, and preferably the activity is increased non-targeted.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product non-targeted with the activity of a “pyruvate kinase”, preferably being encoded by a gene comprising the nucleic acid sequence SEQ ID NO. 26434, preferably the coding region thereof, conferred the production of or the increase in glutamate compared with the wild type control.

The nucleic acid sequence of AvinDRAFT_{—}2091 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of beta-hydroxylase.

Accordingly, in one embodiment, the process of the present invention for producing arginine in a non-human organism, like a microorganism or a plant or a part thereof, comprises increasing or generating the activity of a gene product with the activity of a gene product conferring the activity of “beta-hydroxylase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}2091, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}2091, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}2091, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}2091, and preferably the activity is increased non-targeted.

Accordingly, in one embodiment, the molecule which activity is to be increased in the process of the invention is the gene product with an activity as a “beta-hydroxylase”, preferably it is the molecule of section (a) or (b) of this paragraph.

In particular, it was observed that in plants, especially in Arabidopsis thaliana, increasing or generating the activity of a gene product non-targeted with the activity of a “beta-hydroxylase”, preferably being encoded by a gene comprising the nucleic acid sequence SEQ ID NO. 6040, preferably the coding region thereof, conferred the production of or the increase in arginine compared with the wild type control.

The nucleic acid sequence of AvinDRAFT_{—}2344 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of elongation factor Tu.

Accordingly, in one embodiment, the process of the present invention for producing arginine in a non-human organism, like a microorganism or a plant or a part thereof, comprises increasing or generating the activity of a gene product with the activity of a gene product conferring the activity of “elongation factor Tu”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}2344, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}2344, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}2344, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}2344, and preferably the activity is increased non-targeted.

Accordingly, in one embodiment, the molecule which activity is to be increased in the process of the invention is the gene product with an activity as a “elongation factor Tu”, preferably it is the molecule of section (a) or (b) of this paragraph.

In particular, it was observed that in plants, especially in Arabidopsis thaliana, increasing or generating the activity of a gene product non-targeted with the activity of a “elongation factor Tu”, preferably being encoded by a gene comprising the nucleic acid sequence SEQ ID NO. 27021, preferably the coding region thereof, conferred the production of or the increase in arginine compared with the wild type control.

The nucleic acid sequence of AvinDRAFT_{—}2344 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of elongation factor Tu.

Accordingly, in one embodiment, the process of the present invention for producing glutamate in a non-human organism, like a microorganism or a plant or a part thereof, comprises increasing or generating the activity of a gene product with the activity of a gene product conferring the activity of “elongation factor Tu”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}2344, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}2344, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}2344, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}2344, and preferably the activity is increased non-targeted.

Accordingly, in one embodiment, the molecule which activity is to be increased in the process of the invention is the gene product with an activity as a “elongation factor Tu”, preferably it is the molecule of section (a) or (b) of this paragraph.

In particular, it was observed that in plants, especially in Arabidopsis thaliana, increasing or generating the activity of a gene product non-targeted with the activity of a “elongation factor Tu”, preferably being encoded by a gene comprising the nucleic acid sequence SEQ ID NO. 27021, preferably the coding region thereof, conferred the production of or the increase in glutamate compared with the wild type control.

The nucleic acid sequence of AvinDRAFT_{—}2344 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of elongation factor Tu.

Accordingly, in one embodiment, the process of the present invention for producing glutamine in a non-human organism, like a microorganism or a plant or a part thereof, comprises increasing or generating the activity of a gene product with the activity of a gene product conferring the activity of “elongation factor Tu”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}2344, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}2344, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}2344, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}2344, and preferably the activity is increased non-targeted.

Accordingly, in one embodiment, the molecule which activity is to be increased in the process of the invention is the gene product with an activity as a “elongation factor Tu”, preferably it is the molecule of section (a) or (b) of this paragraph.

In particular, it was observed that in plants, especially in Arabidopsis thaliana, increasing or generating the activity of a gene product non-targeted with the activity of a “elongation factor Tu”, preferably being encoded by a gene comprising the nucleic acid sequence SEQ ID NO. 27021, preferably the coding region thereof, conferred the production of or the increase in glutamine compared with the wild type control.

The nucleic acid sequence of AvinDRAFT_{—}2521 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of ABC transporter permease protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “ABC transporter permease protein”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}2521, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}2521, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}2521, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}2521, and preferably the activity is increased non-targeted.

Accordingly, in one embodiment, the molecule which activity is to be increased in the process of the invention is the gene product with an activity as a “ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “ABC transporter permease protein”, preferably being encoded by a gene comprising the nucleic acid sequence SEQ ID NO. 27882, preferably the coding region thereof, conferred the production of or the increase in glutamine compared with the wild type control.

The nucleic acid sequence of AvinDRAFT_{—}2521 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of ABC transporter permease protein.

Accordingly, in one embodiment, the process of the present invention for producing proline in a non-human organism, like a microorganism or a plant or a part thereof, comprises increasing or generating the activity of a gene product with the activity of a gene product conferring the activity of “ABC transporter permease protein”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}2521, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}2521, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}2521, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}2521, and preferably the activity is increased non-targeted.

Accordingly, in one embodiment, the molecule which activity is to be increased in the process of the invention is the gene product with an activity as a “ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “ABC transporter permease protein”, preferably being encoded by a gene comprising the nucleic acid sequence SEQ ID NO. 27882, preferably the coding region thereof, conferred the production of or the increase in proline compared with the wild type control.

The nucleic acid sequence of AvinDRAFT_{—}2754 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of acyl-CoA synthase.

Accordingly, in one embodiment, the process of the present invention for producing arginine in a non-human organism, like a microorganism or a plant or a part thereof, comprises increasing or generating the activity of a gene product with the activity of a gene product conferring the activity of “acyl-CoA synthase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}2754, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}2754, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}2754, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}2754, and preferably the activity is increased non-targeted.

Accordingly, in one embodiment, the molecule which activity is to be increased in the process of the invention is the gene product with an activity as a “acyl-CoA synthase”, preferably it is the molecule of section (a) or (b) of this paragraph.

In particular, it was observed that in plants, especially in Arabidopsis thaliana, increasing or generating the activity of a gene product non-targeted with the activity of a “acyl-CoA synthase”, preferably being encoded by a gene comprising the nucleic acid sequence SEQ ID NO. 28040, preferably the coding region thereof, conferred the production of or the increase in arginine compared with the wild type control.

The nucleic acid sequence of AvinDRAFT_{—}2754 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of acyl-CoA synthase.

Accordingly, in one embodiment, the process of the present invention for producing glutamate in a non-human organism, like a microorganism or a plant or a part thereof, comprises increasing or generating the activity of a gene product with the activity of a gene product conferring the activity of “acyl-CoA synthase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}2754, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}2754, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}2754, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}2754, and preferably the activity is increased non-targeted.

Accordingly, in one embodiment, the molecule which activity is to be increased in the process of the invention is the gene product with an activity as a “acyl-CoA synthase”, preferably it is the molecule of section (a) or (b) of this paragraph.

In particular, it was observed that in plants, especially in Arabidopsis thaliana, increasing or generating the activity of a gene product non-targeted with the activity of a “acyl-CoA synthase”, preferably being encoded by a gene comprising the nucleic acid sequence SEQ ID NO. 28040, preferably the coding region thereof, conferred the production of or the increase in glutamate compared with the wild type control.

The nucleic acid sequence of AvinDRAFT_{—}2754 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of acyl-CoA synthase.

Accordingly, in one embodiment, the process of the present invention for producing glutamine in a non-human organism, like a microorganism or a plant or a part thereof, comprises increasing or generating the activity of a gene product with the activity of a gene product conferring the activity of “acyl-CoA synthase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}2754, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}2754, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}2754, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}2754, and preferably the activity is increased non-targeted.

Accordingly, in one embodiment, the molecule which activity is to be increased in the process of the invention is the gene product with an activity as a “acyl-CoA synthase”, preferably it is the molecule of section (a) or (b) of this paragraph.

In particular, it was observed that in plants, especially in Arabidopsis thaliana, increasing or generating the activity of a gene product non-targeted with the activity of a “acyl-CoA synthase”, preferably being encoded by a gene comprising the nucleic acid sequence SEQ ID NO. 28040, preferably the coding region thereof, conferred the production of or the increase in glutamine compared with the wild type control.

The nucleic acid sequence of AvinDRAFT_{—}2754 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of acyl-CoA synthase.

Accordingly, in one embodiment, the process of the present invention for producing proline in a non-human organism, like a microorganism or a plant or a part thereof, comprises increasing or generating the activity of a gene product with the activity of a gene product conferring the activity of “acyl-CoA synthase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}2754, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}2754, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}2754, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}2754, and preferably the activity is increased non-targeted.

Accordingly, in one embodiment, the molecule which activity is to be increased in the process of the invention is the gene product with an activity as a “acyl-CoA synthase”, preferably it is the molecule of section (a) or (b) of this paragraph.

In particular, it was observed that in plants, especially in Arabidopsis thaliana, increasing or generating the activity of a gene product non-targeted with the activity of a “acyl-CoA synthase”, preferably being encoded by a gene comprising the nucleic acid sequence SEQ ID NO. 28040, preferably the coding region thereof, conferred the production of or the increase in proline compared with the wild type control.

The nucleic acid sequence of AvinDRAFT_{—}3028 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al And the activity of the gene product thereof is the activity of 2-oxoglutarate dehydrogenase E1 subunit.

Accordingly, in one embodiment, the process of the present invention for producing glutamate in a non-human organism, like a microorganism or a plant or a part thereof, comprises increasing or generating the activity of a gene product with the activity of a gene product conferring the activity of “2-oxoglutarate dehydrogenase E1 subunit”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}3028, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}3028, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}3028, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}3028, and preferably the activity is increased non-targeted.

Accordingly, in one embodiment, the molecule which activity is to be increased in the process of the invention is the gene product with an activity as a “2-oxoglutarate dehydrogenase E1 subunit”, preferably it is the molecule of section (a) or (b) of this paragraph.

In particular, it was observed that in plants, especially in Arabidopsis thaliana, increasing or generating the activity of a gene product non-targeted with the activity of a “2-oxoglutarate dehydrogenase E1 subunit”, preferably being encoded by a gene comprising the nucleic acid sequence SEQ ID NO. 6075, preferably the coding region thereof, conferred the production of or the increase in glutamate compared with the wild type control.

The nucleic acid sequence of AvinDRAFT_{—}3159 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of enoyl-CoA hydratase.

Accordingly, in one embodiment, the process of the present invention for producing arginine in a non-human organism, like a microorganism or a plant or a part thereof, comprises increasing or generating the activity of a gene product with the activity of a gene product conferring the activity of “enoyl-CoA hydratase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}3159, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}3159, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}3159, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}3159, and preferably the activity is increased non-targeted.

Accordingly, in one embodiment, the molecule which activity is to be increased in the process of the invention is the gene product with an activity as a “enoyl-CoA hydratase”, preferably it is the molecule of section (a) or (b) of this paragraph.

In particular, it was observed that in plants, especially in Arabidopsis thaliana, increasing or generating the activity of a gene product non-targeted with the activity of a “enoyl-CoA hydratase”, preferably being encoded by a gene comprising the nucleic acid sequence SEQ ID NO. 28738, preferably the coding region thereof, conferred the production of or the increase in arginine compared with the wild type control.

The nucleic acid sequence of AvinDRAFT_{—}3159 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et a. And the activity of the gene product thereof is the activity of enoyl-CoA hydratase.

Accordingly, in one embodiment, the process of the present invention for producing proline in a non-human organism, like a microorganism or a plant or a part thereof, comprises increasing or generating the activity of a gene product with the activity of a gene product conferring the activity of “enoyl-CoA hydratase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}3159, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}3159, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}3159, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}3159, and preferably the activity is increased non-targeted.

Accordingly, in one embodiment, the molecule which activity is to be increased in the process of the invention is the gene product with an activity as a “enoyl-CoA hydratase”, preferably it is the molecule of section (a) or (b) of this paragraph.

In particular, it was observed that in plants, especially in Arabidopsis thaliana, increasing or generating the activity of a gene product non-targeted with the activity of a “enoyl-CoA hydratase”, preferably being encoded by a gene comprising the nucleic acid sequence SEQ ID NO. 28738, preferably the coding region thereof, conferred the production of or the increase in proline compared with the wild type control.

The nucleic acid sequence of AvinDRAFT_{—}3186 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of transcriptional regulator.

Accordingly, in one embodiment, the process of the present invention for producing glutamate in a non-human organism, like a microorganism or a plant or a part thereof, comprises increasing or generating the activity of a gene product with the activity of a gene product conferring the activity of “transcriptional regulator”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}3186, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}3186, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}3186, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}3186, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 29246, preferably the coding region thereof, conferred the production of or the increase in glutamate compared with the wild type control.

The nucleic acid sequence of AvinDRAFT_{—}3209 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of transcriptional regulator.

Accordingly, in one embodiment, the process of the present invention for producing proline in a non-human organism, like a microorganism or a plant or a part thereof, comprises increasing or generating the activity of a gene product with the activity of a gene product conferring the activity of “transcriptional regulator”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}3209, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}3209, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}3209, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}3209, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 29286, preferably the coding region thereof, conferred the production of or the increase in proline compared with the wild type control.

The nucleic acid sequence of AvinDRAFT_{—}3250 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of acyltransferase.

Accordingly, in one embodiment, the process of the present invention for producing proline in a non-human organism, like a microorganism or a plant or a part thereof, comprises increasing or generating the activity of a gene product with the activity of a gene product conferring the activity of “acyltransferase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}3250, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}3250, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}3250, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}3250, and preferably the activity is increased non-targeted.

Accordingly, in one embodiment, the molecule which activity is to be increased in the process of the invention is the gene product with an activity as a “acyltransferase”, preferably it is the molecule of section (a) or (b) of this paragraph.

In particular, it was observed that in plants, especially in Arabidopsis thaliana, increasing or generating the activity of a gene product non-targeted with the activity of a “acyltransferase”, preferably being encoded by a gene comprising the nucleic acid sequence SEQ ID NO. 29397, preferably the coding region thereof, conferred the production of or the increase in proline compared with the wild type control.

The nucleic acid sequence of AvinDRAFT_{—}3253 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of oxidoreductase.

Accordingly, in one embodiment, the process of the present invention for producing glutamate in a non-human organism, like a microorganism or a plant or a part thereof, comprises increasing or generating the activity of a gene product with the activity of a gene product conferring the activity of “oxidoreductase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}3253, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}3253, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}3253, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}3253, and preferably the activity is increased non-targeted.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 29500, preferably the coding region thereof, conferred the production of or the increase in glutamate compared with the wild type control.

The nucleic acid sequence of AvinDRAFT_{—}3253 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of oxidoreductase.

Accordingly, in one embodiment, the process of the present invention for producing glutamine in a non-human organism, like a microorganism or a plant or a part thereof, comprises increasing or generating the activity of a gene product with the activity of a gene product conferring the activity of “oxidoreductase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}3253, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}3253, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}3253, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}3253, and preferably the activity is increased non-targeted.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 29500, preferably the coding region thereof, conferred the production of or the increase in glutamine compared with the wild type control.

The nucleic acid sequence of AvinDRAFT_{—}3556 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of lysyl-tRNA synthetase.

Accordingly, in one embodiment, the process of the present invention for producing glutamine in a non-human organism, like a microorganism or a plant or a part thereof, comprises increasing or generating the activity of a gene product with the activity of a gene product conferring the activity of “lysyl-tRNA synthetase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}3556, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}3556, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}3556, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}3556, and preferably the activity is increased non-targeted.

Accordingly, in one embodiment, the molecule which activity is to be increased in the process of the invention is the gene product with an activity as a “lysyl-tRNA synthetase”, preferably it is the molecule of section (a) or (b) of this paragraph.

In particular, it was observed that in plants, especially in Arabidopsis thaliana, increasing or generating the activity of a gene product non-targeted with the activity of a “lysyl-tRNA synthetase”, preferably being encoded by a gene comprising the nucleic acid sequence SEQ ID NO. 30039, preferably the coding region thereof, conferred the production of or the increase in glutamine compared with the wild type control.

The nucleic acid sequence of AvinDRAFT_{—}3587 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of 30S ribosomal protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “305 ribosomal protein”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}3587, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}3587, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}3587, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}3587, and preferably the activity is increased non-targeted.

Accordingly, in one embodiment, the molecule which activity is to be increased in the process of the invention is the gene product with an activity as a “305 ribosomal protein”, preferably it is the molecule of section (a) or (b) of this paragraph.

In particular, it was observed that in plants, especially in Arabidopsis thaliana, increasing or generating the activity of a gene product non-targeted with the activity of a “305 ribosomal protein”, preferably being encoded by a gene comprising the nucleic acid sequence SEQ ID NO. 30464, preferably the coding region thereof, conferred the production of or the increase in glutamate compared with the wild type control.

The nucleic acid sequence of AvinDRAFT_{—}3605 from Azotobacter vinelandii, e.g. as shown in the respective line in column 5 of Table I, application no. 2, has been published deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of CTP synthase.

Accordingly, in one embodiment, the process of the present invention for producing glutamate in a non-human organism, like a microorganism or a plant or a part thereof, comprises increasing or generating the activity of a gene product with the activity of a gene product conferring the activity of “CTP synthase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}3605, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}3605, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}3605, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}3605, and preferably the activity is increased non-targeted.

Accordingly, in one embodiment, the molecule which activity is to be increased in the process of the invention is the gene product with an activity as a “CTP synthase”, preferably it is the molecule of section (a) or (b) of this paragraph.

In particular, it was observed that in plants, especially in Arabidopsis thaliana, increasing or generating the activity of a gene product non-targeted with the activity of a “CTP synthase”, preferably being encoded by a gene comprising the nucleic acid sequence SEQ ID NO. 31026, preferably the coding region thereof, conferred the production of or the increase in glutamate compared with the wild type control.

The nucleic acid sequence of AvinDRAFT_{—}3605 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of CTP synthase.

Accordingly, in one embodiment, the process of the present invention for producing glutamine in a non-human organism, like a microorganism or a plant or a part thereof, comprises increasing or generating the activity of a gene product with the activity of a gene product conferring the activity of “CTP synthase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}3605, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}3605, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}3605, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}3605, and preferably the activity is increased non-targeted.

Accordingly, in one embodiment, the molecule which activity is to be increased in the process of the invention is the gene product with an activity as a “CTP synthase”, preferably it is the molecule of section (a) or (b) of this paragraph.

In particular, it was observed that in plants, especially in Arabidopsis thaliana, increasing or generating the activity of a gene product non-targeted with the activity of a “CTP synthase”, preferably being encoded by a gene comprising the nucleic acid sequence SEQ ID NO. 31026, preferably the coding region thereof, conferred the production of or the increase in glutamine compared with the wild type control.

The nucleic acid sequence of AvinDRAFT_{—}4384 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of glucose-1-phosphate cytidylyltransferase.

Accordingly, in one embodiment, the process of the present invention for producing glutamate in a non-human organism, like a microorganism or a plant or a part thereof, comprises increasing or generating the activity of a gene product with the activity of a gene product conferring the activity of “glucose-1-phosphate cytidylyltransferase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}4384, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}4384, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}4384, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}4384, and preferably the activity is increased non-targeted.

Accordingly, in one embodiment, the molecule which activity is to be increased in the process of the invention is the gene product with an activity as a “glucose-1-phosphate cytidylyltransferase”, preferably it is the molecule of section (a) or (b) of this paragraph.

In particular, it was observed that in plants, especially in Arabidopsis thaliana, increasing or generating the activity of a gene product non-targeted with the activity of a “glucose-1-phosphate cytidylyltransferase”, preferably being encoded by a gene comprising the nucleic acid sequence SEQ ID NO. 31717, preferably the coding region thereof, conferred the production of or the increase in glutamate compared with the wild type control.

The nucleic acid sequence of AvinDRAFT_{—}4384 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of glucose-1-phosphate cytidylyltransferase.

Accordingly, in one embodiment, the process of the present invention for producing glutamine in a non-human organism, like a microorganism or a plant or a part thereof, comprises increasing or generating the activity of a gene product with the activity of a gene product conferring the activity of “glucose-1-phosphate cytidylyltransferase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}4384, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}4384, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}4384, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}4384, and preferably the activity is increased non-targeted.

Accordingly, in one embodiment, the molecule which activity is to be increased in the process of the invention is the gene product with an activity as a “glucose-1-phosphate cytidylyltransferase”, preferably it is the molecule of section (a) or (b) of this paragraph.

In particular, it was observed that in plants, especially in Arabidopsis thaliana, increasing or generating the activity of a gene product non-targeted with the activity of a “glucose-1-phosphate cytidylyltransferase”, preferably being encoded by a gene comprising the nucleic acid sequence SEQ ID NO. 31717, preferably the coding region thereof, conferred the production of or the increase in glutamine compared with the wild type control.

The nucleic acid sequence of AvinDRAFT_{—}4384 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of glucose-1-phosphate cytidylyltransferase.

Accordingly, in one embodiment, the process of the present invention for producing proline in a non-human organism, like a microorganism or a plant or a part thereof, comprises increasing or generating the activity of a gene product with the activity of a gene product conferring the activity of “glucose-1-phosphate cytidylyltransferase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}4384, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}4384, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}4384, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}4384, and preferably the activity is increased non-targeted.

Accordingly, in one embodiment, the molecule which activity is to be increased in the process of the invention is the gene product with an activity as a “glucose-1-phosphate cytidylyltransferase”, preferably it is the molecule of section (a) or (b) of this paragraph.

In particular, it was observed that in plants, especially in Arabidopsis thaliana, increasing or generating the activity of a gene product non-targeted with the activity of a “glucose-1-phosphate cytidylyltransferase”, preferably being encoded by a gene comprising the nucleic acid sequence SEQ ID NO. 31717, preferably the coding region thereof, conferred the production of or the increase in proline compared with the wild type control.

The nucleic acid sequence of AvinDRAFT_{—}4562 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of aminotransferase.

Accordingly, in one embodiment, the process of the present invention for producing arginine in a non-human organism, like a microorganism or a plant or a part thereof, comprises increasing or generating the activity of a gene product with the activity of a gene product conferring the activity of “aminotransferase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}4562, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}4562, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}4562, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}4562, and preferably the activity is increased non-targeted.

Accordingly, in one embodiment, the molecule which activity is to be increased in the process of the invention is the gene product with an activity as a “aminotransferase”, preferably it is the molecule of section (a) or (b) of this paragraph.

In particular, it was observed that in plants, especially in Arabidopsis thaliana, increasing or generating the activity of a gene product non-targeted with the activity of a “aminotransferase”, preferably being encoded by a gene comprising the nucleic acid sequence SEQ ID NO. 31926, preferably the coding region thereof, conferred the production of or the increase in arginine compared with the wild type control.

The nucleic acid sequence of AvinDRAFT_{—}5103 from Azotobacter vinelandii, e.g. as shown in the respective line in column 5 of Table I, application no. 2, has been published deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of hydrolase.

Accordingly, in one embodiment, the process of the present invention for producing proline in a non-human organism, like a microorganism or a plant or a part thereof, comprises increasing or generating the activity of a gene product with the activity of a gene product conferring the activity of “hydrolase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}5103, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}5103, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}5103, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}5103, and preferably the activity is increased non-targeted.

Accordingly, in one embodiment, the molecule which activity is to be increased in the process of the invention is the gene product with an activity as a “hydrolase”, preferably it is the molecule of section (a) or (b) of this paragraph.

In particular, it was observed that in plants, especially in Arabidopsis thaliana, increasing or generating the activity of a gene product non-targeted with the activity of a “hydrolase”, preferably being encoded by a gene comprising the nucleic acid sequence SEQ ID NO. 6510, preferably the coding region thereof, conferred the production of or the increase in proline compared with the wild type control.

The nucleic acid sequence of AvinDRAFT_{—}5246 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of glutamate-ammonia-ligase.

Accordingly, in one embodiment, the process of the present invention for producing arginine in a non-human organism, like a microorganism or a plant or a part thereof, comprises increasing or generating the activity of a gene product with the activity of a gene product conferring the activity of “glutamate-ammonia-ligase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}5246, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}5246, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}5246, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}5246, and preferably the activity is increased non-targeted.

Accordingly, in one embodiment, the molecule which activity is to be increased in the process of the invention is the gene product with an activity as a “glutamate-ammonia-ligase”, preferably it is the molecule of section (a) or (b) of this paragraph.

In particular, it was observed that in plants, especially in Arabidopsis thaliana, increasing or generating the activity of a gene product non-targeted with the activity of a “glutamate-ammonia-ligase”, preferably being encoded by a gene comprising the nucleic acid sequence SEQ ID NO. 32037, preferably the coding region thereof, conferred the production of or the increase in arginine compared with the wild type control.

The nucleic acid sequence of AvinDRAFT_{—}5246 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of glutamate-ammonia-ligase.

Accordingly, in one embodiment, the process of the present invention for producing glutamine in a non-human organism, like a microorganism or a plant or a part thereof, comprises increasing or generating the activity of a gene product with the activity of a gene product conferring the activity of “glutamate-ammonia-ligase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}5246, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}5246, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}5246, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}5246, and preferably the activity is increased non-targeted.

Accordingly, in one embodiment, the molecule which activity is to be increased in the process of the invention is the gene product with an activity as a “glutamate-ammonia-ligase”, preferably it is the molecule of section (a) or (b) of this paragraph.

In particular, it was observed that in plants, especially in Arabidopsis thaliana, increasing or generating the activity of a gene product non-targeted with the activity of a “glutamate-ammonia-ligase”, preferably being encoded by a gene comprising the nucleic acid sequence SEQ ID NO. 32037, preferably the coding region thereof, conferred the production of or the increase in glutamine compared with the wild type control.

The nucleic acid sequence of AvinDRAFT_{—}5246 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of glutamate-ammonia-ligase.

Accordingly, in one embodiment, the process of the present invention for producing proline in a non-human organism, like a microorganism or a plant or a part thereof, comprises increasing or generating the activity of a gene product with the activity of a gene product conferring the activity of “glutamate-ammonia-ligase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}5246, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}5246, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}5246, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}5246, and preferably the activity is increased non-targeted.

Accordingly, in one embodiment, the molecule which activity is to be increased in the process of the invention is the gene product with an activity as a “glutamate-ammonia-ligase”, preferably it is the molecule of section (a) or (b) of this paragraph.

In particular, it was observed that in plants, especially in Arabidopsis thaliana, increasing or generating the activity of a gene product non-targeted with the activity of a “glutamate-ammonia-ligase”, preferably being encoded by a gene comprising the nucleic acid sequence SEQ ID NO. 32037, preferably the coding region thereof, conferred the production of or the increase in proline compared with the wild type control.

The nucleic acid sequence of AvinDRAFT_{—}5292 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of fumarylacetoacetate hydrolase.

Accordingly, in one embodiment, the process of the present invention for producing proline in a non-human organism, like a microorganism or a plant or a part thereof, comprises increasing or generating the activity of a gene product with the activity of a gene product conferring the activity of “fumarylacetoacetate hydrolase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}5292, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}5292, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}5292, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}5292, and preferably the activity is increased non-targeted.

Accordingly, in one embodiment, the molecule which activity is to be increased in the process of the invention is the gene product with an activity as a “fumarylacetoacetate hydrolase”, preferably it is the molecule of section (a) or (b) of this paragraph.

In particular, it was observed that in plants, especially in Arabidopsis thaliana, increasing or generating the activity of a gene product non-targeted with the activity of a “fumarylacetoacetate hydrolase”, preferably being encoded by a gene comprising the nucleic acid sequence SEQ ID NO. 32308, preferably the coding region thereof, conferred the production of or the increase in proline compared with the wild type control.

The nucleic acid sequence of AvinDRAFT_{—}5467 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of HesB/YadR/YfhF family protein.

Accordingly, in one embodiment, the process of the present invention for producing arginine in a non-human organism, like a microorganism or a plant or a part thereof, comprises increasing or generating the activity of a gene product with the activity of a gene product conferring the activity of “HesB/YadR/YfhF family protein”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}5467, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}5467, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}5467, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}5467, and preferably the activity is increased non-targeted.

Accordingly, in one embodiment, the molecule which activity is to be increased in the process of the invention is the gene product with an activity as a “HesB/YadR/YfhF family protein”, preferably it is the molecule of section (a) or (b) of this paragraph.

In particular, it was observed that in plants, especially in Arabidopsis thaliana, increasing or generating the activity of a gene product non-targeted with the activity of a “HesB/YadR/YfhF family protein”, preferably being encoded by a gene comprising the nucleic acid sequence SEQ ID NO. 32648, preferably the coding region thereof, conferred the production of or the increase in arginine compared with the wild type control.

The nucleic acid sequence of AvinDRAFT_{—}5467 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of HesB/YadR/YfhF family protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “HesB/YadR/YfhF family protein”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}5467, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}5467, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}5467, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}5467, and preferably the activity is increased non-targeted.

Accordingly, in one embodiment, the molecule which activity is to be increased in the process of the invention is the gene product with an activity as a “HesB/YadR/YfhF family protein”, preferably it is the molecule of section (a) or (b) of this paragraph.

In particular, it was observed that in plants, especially in Arabidopsis thaliana, increasing or generating the activity of a gene product non-targeted with the activity of a “HesB/YadR/YfhF family protein”, preferably being encoded by a gene comprising the nucleic acid sequence SEQ ID NO. 32648, preferably the coding region thereof, conferred the production of or the increase in glutamine compared with the wild type control.

The nucleic acid sequence of AvinDRAFT_{—}5467 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al And the activity of the gene product thereof is the activity of HesB/YadR/YfhF family protein.

Accordingly, in one embodiment, the process of the present invention for producing proline in a non-human organism, like a microorganism or a plant or a part thereof, comprises increasing or generating the activity of a gene product with the activity of a gene product conferring the activity of “HesB/YadR/YfhF family protein”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}5467, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}5467, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}5467, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}5467, and preferably the activity is increased non-targeted.

Accordingly, in one embodiment, the molecule which activity is to be increased in the process of the invention is the gene product with an activity as a “HesB/YadR/YfhF family protein”, preferably it is the molecule of section (a) or (b) of this paragraph.

In particular, it was observed that in plants, especially in Arabidopsis thaliana, increasing or generating the activity of a gene product non-targeted with the activity of a “HesB/YadR/YfhF family protein”, preferably being encoded by a gene comprising the nucleic acid sequence SEQ ID NO. 32648, preferably the coding region thereof, conferred the production of or the increase in proline compared with the wild type control.

The nucleic acid sequence of AvinDRAFT_{—}5644 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al And the activity of the gene product thereof is the activity of L-aspartate oxidase.

Accordingly, in one embodiment, the process of the present invention for producing proline in a non-human organism, like a microorganism or a plant or a part thereof, comprises increasing or generating the activity of a gene product with the activity of a gene product conferring the activity of “L-aspartate oxidase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}5644, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}5644, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}5644, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}5644, and preferably the activity is increased non-targeted.

Accordingly, in one embodiment, the molecule which activity is to be increased in the process of the invention is the gene product with an activity as a “L-aspartate oxidase”, preferably it is the molecule of section (a) or (b) of this paragraph.

In particular, it was observed that in plants, especially in Arabidopsis thaliana, increasing or generating the activity of a gene product non-targeted with the activity of a “L-aspartate oxidase”, preferably being encoded by a gene comprising the nucleic acid sequence SEQ ID NO. 33085, preferably the coding region thereof, conferred the production of or the increase in proline compared with the wild type control.

The nucleic acid sequence of AvinDRAFT_{—}5651 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al And the activity of the gene product thereof is the activity of oxidoreductase.

Accordingly, in one embodiment, the process of the present invention for producing arginine in a non-human organism, like a microorganism or a plant or a part thereof, comprises increasing or generating the activity of a gene product with the activity of a gene product conferring the activity of “oxidoreductase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}5651, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}5651, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}5651, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}5651, and preferably the activity is increased non-targeted.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 33457, preferably the coding region thereof, conferred the production of or the increase in arginine compared with the wild type control.

The nucleic acid sequence of AvinDRAFT_{—}5651 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of oxidoreductase.

Accordingly, in one embodiment, the process of the present invention for producing glutamine in a non-human organism, like a microorganism or a plant or a part thereof, comprises increasing or generating the activity of a gene product with the activity of a gene product conferring the activity of “oxidoreductase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}5651, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}5651, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}5651, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}5651, and preferably the activity is increased non-targeted.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 33457, preferably the coding region thereof, conferred the production of or the increase in glutamine compared with the wild type control.

The nucleic acid sequence of AvinDRAFT_{—}6093 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of Chaperone protein CIpB.

Accordingly, in one embodiment, the process of the present invention for producing proline in a non-human organism, like a microorganism or a plant or a part thereof, comprises increasing or generating the activity of a gene product with the activity of a gene product conferring the activity of “Chaperone protein CIpB”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}6093, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}6093, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}6093, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}6093, and preferably the activity is increased non-targeted.

Accordingly, in one embodiment, the molecule which activity is to be increased in the process of the invention is the gene product with an activity as a “Chaperone protein CIpB”, preferably it is the molecule of section (a) or (b) of this paragraph.

In particular, it was observed that in plants, especially in Arabidopsis thaliana, increasing or generating the activity of a gene product non-targeted with the activity of a “Chaperone protein CIpB”, preferably being encoded by a gene comprising the nucleic acid sequence SEQ ID NO. 33596, preferably the coding region thereof, conferred the production of or the increase in proline compared with the wild type control.

The nucleic acid sequence of AvinDRAFT_{—}6700 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of purine nucleoside phosphorylase.

Accordingly, in one embodiment, the process of the present invention for producing arginine in a non-human organism, like a microorganism or a plant or a part thereof, comprises increasing or generating the activity of a gene product with the activity of a gene product conferring the activity of “purine nucleoside phosphorylase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}6700, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}6700, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}6700, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}6700, and preferably the activity is increased non-targeted.

Accordingly, in one embodiment, the molecule which activity is to be increased in the process of the invention is the gene product with an activity as a “purine nucleoside phosphorylase”, preferably it is the molecule of section (a) or (b) of this paragraph.

In particular, it was observed that in plants, especially in Arabidopsis thaliana, increasing or generating the activity of a gene product non-targeted with the activity of a “purine nucleoside phosphorylase”, preferably being encoded by a gene comprising the nucleic acid sequence SEQ ID NO. 34044, preferably the coding region thereof, conferred the production of or the increase in arginine compared with the wild type control.

The nucleic acid sequence of AvinDRAFT_{—}6700 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of purine nucleoside phosphorylase.

Accordingly, in one embodiment, the process of the present invention for producing glutamine in a non-human organism, like a microorganism or a plant or a part thereof, comprises increasing or generating the activity of a gene product with the activity of a gene product conferring the activity of “purine nucleoside phosphorylase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}6700, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}6700, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}6700, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}6700, and preferably the activity is increased non-targeted.

Accordingly, in one embodiment, the molecule which activity is to be increased in the process of the invention is the gene product with an activity as a “purine nucleoside phosphorylase”, preferably it is the molecule of section (a) or (b) of this paragraph.

In particular, it was observed that in plants, especially in Arabidopsis thaliana, increasing or generating the activity of a gene product non-targeted with the activity of a “purine nucleoside phosphorylase”, preferably being encoded by a gene comprising the nucleic acid sequence SEQ ID NO. 34044, preferably the coding region thereof, conferred the production of or the increase in glutamine compared with the wild type control.

The nucleic acid sequence of AvinDRAFT_{—}6700 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of purine nucleoside phosphorylase.

Accordingly, in one embodiment, the process of the present invention for producing proline in a non-human organism, like a microorganism or a plant or a part thereof, comprises increasing or generating the activity of a gene product with the activity of a gene product conferring the activity of “purine nucleoside phosphorylase”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}6700, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}6700, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}6700, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}6700, and preferably the activity is increased non-targeted.

Accordingly, in one embodiment, the molecule which activity is to be increased in the process of the invention is the gene product with an activity as a “purine nucleoside phosphorylase”, preferably it is the molecule of section (a) or (b) of this paragraph.

In particular, it was observed that in plants, especially in Arabidopsis thaliana, increasing or generating the activity of a gene product non-targeted with the activity of a “purine nucleoside phosphorylase”, preferably being encoded by a gene comprising the nucleic acid sequence SEQ ID NO. 34044, preferably the coding region thereof, conferred the production of or the increase in proline compared with the wild type control.

The nucleic acid sequence of AvinDRAFT_{—}6864 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of transcriptional regulator protein.

Accordingly, in one embodiment, the process of the present invention for producing proline in a non-human organism, like a microorganism or a plant or a part thereof, comprises increasing or generating the activity of a gene product with the activity of a gene product conferring the activity of “transcriptional regulator protein”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AvinDRAFT_{—}6864, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AvinDRAFT_{—}6864, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AvinDRAFT_{—}6864, or a functional equivalent or a homolog thereof as depicted in column 8 of Table II, application no. 2, preferably a homolog or functional equivalent as depicted in column 8 of Table II B, application no. 2, and being depicted in the same respective line as said AvinDRAFT_{—}6864, and preferably the activity is increased non-targeted.

Accordingly, in one embodiment, the molecule which activity is to be increased in the process of the invention is the gene product with an activity as a “transcriptional regulator protein”, preferably it is the molecule of section (a) or (b) of this paragraph. In particular, it was observed that in plants, especially in Arabidopsis thaliana, increasing or generating the activity of a gene product non-targeted with the activity of a “transcriptional regulator protein”, preferably being encoded by a gene comprising the nucleic acid sequence SEQ ID NO. 34204, preferably the coding region thereof, conferred the production of or the increase in proline compared with the wild type control.

The nucleic acid sequence of AX653549 from Oryza sativa, e.g. as shown in the respective line in column 5 of Table I, application no. 2 is unpublished. And the activity of the gene product thereof is the activity of AX653549-protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “AX653549-protein”, especially from Oryza sativa or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AX653549, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AX653549, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AX653549, or a functional equivalent or a homolog thereof as depicted in column 8 of Table II, application no. 2, preferably a homolog or functional equivalent as depicted in column 8 of Table II B, application no. 2, and being depicted in the same respective line as said AX653549, and preferably the activity is increased non-targeted.

Accordingly, in one embodiment, the molecule which activity is to be increased in the process of the invention is the gene product with an activity as a “AX653549-protein”, preferably it is the molecule of section (a) or (b) of this paragraph.

In particular, it was observed that in plants, especially in Arabidopsis thaliana, increasing or generating the activity of a gene product non-targeted with the activity of a “AX653549-protein”, preferably being encoded by a gene comprising the nucleic acid sequence SEQ ID NO. 34301, preferably the coding region thereof, conferred the production of or the increase in glutamate compared with the wild type control.

The nucleic acid sequence of AX653549 from Oryza sativa, e.g. as shown in the respective line in column 5 of Table I, application no. 2 is unpublished. And the activity of the gene product thereof is the activity of AX653549-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “AX653549-protein”, especially from Oryza sativa or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AX653549, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AX653549, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AX653549, or a functional equivalent or a homolog thereof as depicted in column 8 of Table II, application no. 2, preferably a homolog or functional equivalent as depicted in column 8 of Table II B, application no. 2, and being depicted in the same respective line as said AX653549, and preferably the activity is increased non-targeted.

Accordingly, in one embodiment, the molecule which activity is to be increased in the process of the invention is the gene product with an activity as a “AX653549-protein”, preferably it is the molecule of section (a) or (b) of this paragraph.

In particular, it was observed that in plants, especially in Arabidopsis thaliana, increasing or generating the activity of a gene product non-targeted with the activity of a “AX653549-protein”, preferably being encoded by a gene comprising the nucleic acid sequence SEQ ID NO. 34301, preferably the coding region thereof, conferred the production of or the increase in glutamine compared with the wild type control.

The nucleic acid sequence of AY087308 from Arabidopsis thaliana, e.g. as shown in the respective line in column 5 of Table I, application no. 2 is unpublished. And the activity of the gene product thereof is the activity of AY087308-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “AY087308-protein”, especially from Arabidopsis thaliana or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said AY087308, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said AY087308, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said AY087308, or a functional equivalent or a homolog thereof as depicted in column 8 of Table II, application no. 2, preferably a homolog or functional equivalent as depicted in column 8 of Table II B, application no. 2, and being depicted in the same respective line as said AY087308, and preferably the activity is increased non-targeted.

Accordingly, in one embodiment, the molecule which activity is to be increased in the process of the invention is the gene product with an activity as a “AY087308-protein”, preferably it is the molecule of section (a) or (b) of this paragraph. In particular, it was observed that in plants, especially in Arabidopsis thaliana, increasing or generating the activity of a gene product non-targeted with the activity of a “AY087308-protein”, preferably being encoded by a gene comprising the nucleic acid sequence SEQ ID NO. 34602, preferably the coding region thereof, conferred the production of or the increase in glutamine compared with the wild type control.

The nucleic acid sequence of B0004 from Escherichia coli, e.g. as shown in the respective line in column 5 of Table I, application no. 2, has been published in Blattner et al., Science 277 (5331). And the activity of the gene product thereof is the activity of threonine synthase.

Accordingly, in one embodiment, the process of the present invention for producing arginine in a non-human organism, like a microorganism or a plant or a part thereof, comprises increasing or generating the activity of a gene product with the activity of a gene product conferring the activity of “threonine synthase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0004, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0004, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0004, or a functional equivalent or a homolog thereof as depicted in column 8 of Table II, application no. 2, preferably a homolog or functional equivalent as depicted in column 8 of Table II B, application no. 2, and being depicted in the same respective line as said B0004, and preferably the activity is increased plastidic.

Accordingly, in one embodiment, the molecule which activity is to be increased in the process of the invention is the gene product with an activity as a “threonine synthase”, preferably it is the molecule of section (a) or (b) of this paragraph.

In particular, it was observed that in plants, especially in Arabidopsis thaliana, increasing or generating the activity of a gene product plastidic with the activity of a “threonine synthase”, preferably being encoded by a gene comprising the nucleic acid sequence SEQ ID NO. 34889, preferably the coding region thereof, conferred the production of or the increase in arginine compared with the wild type control.

The nucleic acid sequence of B0004 from Escherichia coli, e.g. as shown in the respective line in column 5 of Table I, application no. 2, has been published in Blattner et al., Science 277 (5331), 1453 (1997)). And the activity of the gene product thereof is the activity of threonine synthase.

Accordingly, in one embodiment, the process of the present invention for producing glutamine in a non-human organism, like a microorganism or a plant or a part thereof, comprises increasing or generating the activity of a gene product with the activity of a gene product conferring the activity of “threonine synthase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0004, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0004, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0004, or a functional equivalent or a homolog thereof as depicted in column 8 of Table II, application no. 2, preferably a homolog or functional equivalent as depicted in column 8 of Table II B, application no. 2, and being depicted in the same respective line as said B0004, and preferably the activity is increased plastidic.

Accordingly, in one embodiment, the molecule which activity is to be increased in the process of the invention is the gene product with an activity as a “threonine synthase”, preferably it is the molecule of section (a) or (b) of this paragraph.

In particular, it was observed that in plants, especially in Arabidopsis thaliana, increasing or generating the activity of a gene product plastidic with the activity of a “threonine synthase”, preferably being encoded by a gene comprising the nucleic acid sequence SEQ ID NO. 34889, preferably the coding region thereof, conferred the production of or the increase in glutamine compared with the wild type control.

The nucleic acid sequence of B0004 from Escherichia coli, e.g. as shown in the respective line in column 5 of Table I, application no. 2, has been published in Blattner et al., Science 277 (5331), 1453 (1997)). And the activity of the gene product thereof is the activity of threonine synthase.

Accordingly, in one embodiment, the process of the present invention for producing proline in a non-human organism, like a microorganism or a plant or a part thereof, comprises increasing or generating the activity of a gene product with the activity of a gene product conferring the activity of “threonine synthase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0004, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0004, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0004, or a functional equivalent or a homolog thereof as depicted in column 8 of Table II, application no. 2, preferably a homolog or functional equivalent as depicted in column 8 of Table II B, application no. 2, and being depicted in the same respective line as said B0004, and preferably the activity is increased plastidic.

Accordingly, in one embodiment, the molecule which activity is to be increased in the process of the invention is the gene product with an activity as a “threonine synthase”, preferably it is the molecule of section (a) or (b) of this paragraph.

In particular, it was observed that in plants, especially in Arabidopsis thaliana, increasing or generating the activity of a gene product plastidic with the activity of a “threonine synthase”, preferably being encoded by a gene comprising the nucleic acid sequence SEQ ID NO. 34889, preferably the coding region thereof, conferred the production of or the increase in proline compared with the wild type control.

The nucleic acid sequence of B0061 from Escherichia coli, e.g. as shown in the respective line in column 5 of Table I, application no. 2, has been published in Blattner et al., Science 277 (5331), 1453 (1997). And the activity of the gene product thereof is the activity of L-ribulose-5-phosphate 4-epimerase.

Accordingly, in one embodiment, the process of the present invention for producing proline in a non-human organism, like a microorganism or a plant or a part thereof, comprises increasing or generating the activity of a gene product with the activity of a gene product conferring the activity of “L-ribulose-5-phosphate 4-epimerase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0061, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0061, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0061, or a functional equivalent or a homolog thereof as depicted in column 8 of Table II, application no. 2, preferably a homolog or functional equivalent as depicted in column 8 of Table II B, application no. 2, and being depicted in the same respective line as said B0061, and preferably the activity is increased non-targeted.

Accordingly, in one embodiment, the molecule which activity is to be increased in the process of the invention is the gene product with an activity as a “L-ribulose-5-phosphate 4-epimerase”, preferably it is the molecule of section (a) or (b) of this paragraph.

In particular, it was observed that in plants, especially in Arabidopsis thaliana, increasing or generating the activity of a gene product non-targeted with the activity of a “L-ribulose-5-phosphate 4-epimerase”, preferably being encoded by a gene comprising the nucleic acid sequence SEQ ID NO. 35204, preferably the coding region thereof, conferred the production of or the increase in proline compared with the wild type control.

The nucleic acid sequence of B0115 from Escherichia coli, e.g. as shown in the respective line in column 5 of Table I, application no. 2, has been published in Blattner et al., Science 277 (5331), 1453 (1997)). And the activity of the gene product thereof is the activity of dihydrolipoamide acetyltransferase.

Accordingly, in one embodiment, the process of the present invention for producing glutamine in a non-human organism, like a microorganism or a plant or a part thereof, comprises increasing or generating the activity of a gene product with the activity of a gene product conferring the activity of “dihydrolipoamide acetyltransferase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0115, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0115, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0115, or a functional equivalent or a homolog thereof as depicted in column 8 of Table II, application no. 2, preferably a homolog or functional equivalent as depicted in column 8 of Table II B, application no. 2, and being depicted in the same respective line as said B0115, and preferably the activity is increased plastidic.

Accordingly, in one embodiment, the molecule which activity is to be increased in the process of the invention is the gene product with an activity as a “dihydrolipoamide acetyltransferase”, preferably it is the molecule of section (a) or (b) of this paragraph.

In particular, it was observed that in plants, especially in Arabidopsis thaliana, increasing or generating the activity of a gene product plastidic with the activity of a “dihydrolipoamide acetyltransferase”, preferably being encoded by a gene comprising the nucleic acid sequence SEQ ID NO. 35366, preferably the coding region thereof, conferred the production of or the increase in glutamine compared with the wild type control.

The nucleic acid sequence of B0124 from Escherichia coli, e.g. as shown in the respective line in column 5 of Table I, application no. 2, has been published in Blattner et al., Science 277 (5331), 1453 (1997)). And the activity of the gene product thereof is the activity of glucose dehydrogenase.

Accordingly, in one embodiment, the process of the present invention for producing arginine in a non-human organism, like a microorganism or a plant or a part thereof, comprises increasing or generating the activity of a gene product with the activity of a gene product conferring the activity of “glucose dehydrogenase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0124, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0124, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0124, or a functional equivalent or a homolog thereof as depicted in column 8 of Table II, application no. 2, preferably a homolog or functional equivalent as depicted in column 8 of Table II B, application no. 2, and being depicted in the same respective line as said B0124, and preferably the activity is increased non-targeted.

Accordingly, in one embodiment, the molecule which activity is to be increased in the process of the invention is the gene product with an activity as a “glucose dehydrogenase”, preferably it is the molecule of section (a) or (b) of this paragraph.

In particular, it was observed that in plants, especially in Arabidopsis thaliana, increasing or generating the activity of a gene product non-targeted with the activity of a “glucose dehydrogenase”, preferably being encoded by a gene comprising the nucleic acid sequence SEQ ID NO. 35482, preferably the coding region thereof, conferred the production of or the increase in arginine compared with the wild type control.

The nucleic acid sequence of B0124 from Escherichia coli, e.g. as shown in the respective line in column 5 of Table I, application no. 2, has been published in Blattner et al., Science 277 (5331), 1453 (1997)). And the activity of the gene product thereof is the activity of glucose dehydrogenase.

Accordingly, in one embodiment, the process of the present invention for producing proline in a non-human organism, like a microorganism or a plant or a part thereof, comprises increasing or generating the activity of a gene product with the activity of a gene product conferring the activity of “glucose dehydrogenase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0124, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0124, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0124, or a functional equivalent or a homolog thereof as depicted in column 8 of Table II, application no. 2, preferably a homolog or functional equivalent as depicted in column 8 of Table II B, application no. 2, and being depicted in the same respective line as said B0124, and preferably the activity is increased plastidic.

Accordingly, in one embodiment, the molecule which activity is to be increased in the process of the invention is the gene product with an activity as a “glucose dehydrogenase”, preferably it is the molecule of section (a) or (b) of this paragraph.

In particular, it was observed that in plants, especially in Arabidopsis thaliana, increasing or generating the activity of a gene product plastidic with the activity of a “glucose dehydrogenase”, preferably being encoded by a gene comprising the nucleic acid sequence SEQ ID NO. 35482, preferably the coding region thereof, conferred the production of or the increase in proline compared with the wild type control.

The nucleic acid sequence of B0124 from Escherichia coli, e.g. as shown in the respective line in column 5 of Table I, application no. 2, has been published in Blattner et al., Science 277 (5331), 1453 (1997)). And the activity of the gene product thereof is the activity of glucose dehydrogenase.

Accordingly, in one embodiment, the process of the present invention for producing proline in a non-human organism, like a microorganism or a plant or a part thereof, comprises increasing or generating the activity of a gene product with the activity of a gene product conferring the activity of “glucose dehydrogenase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0124, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0124, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0124, or a functional equivalent or a homolog thereof as depicted in column 8 of Table II, application no. 2, preferably a homolog or functional equivalent as depicted in column 8 of Table II B, application no. 2, and being depicted in the same respective line as said B0124, and preferably the activity is increased non-targeted.

Accordingly, in one embodiment, the molecule which activity is to be increased in the process of the invention is the gene product with an activity as a “glucose dehydrogenase”, preferably it is the molecule of section (a) or (b) of this paragraph.

In particular, it was observed that in plants, especially in Arabidopsis thaliana, increasing or generating the activity of a gene product non-targeted with the activity of a “glucose dehydrogenase”, preferably being encoded by a gene comprising the nucleic acid sequence SEQ ID NO. 35482, preferably the coding region thereof, conferred the production of or the increase in proline compared with the wild type control.

The nucleic acid sequence of B0161 from Escherichia coli, e.g. as shown in the respective line in column 5 of Table I, application no. 2, has been published in Blattner et al., Science 277 (5331), 1453 (1997)). And the activity of the gene product thereof is the activity of serine protease.

Accordingly, in one embodiment, the process of the present invention for producing proline in a non-human organism, like a microorganism or a plant or a part thereof, comprises increasing or generating the activity of a gene product with the activity of a gene product conferring the activity of “serine protease”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0161, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0161, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0161, or a functional equivalent or a homolog thereof as depicted in column 8 of Table II, application no. 2, preferably a homolog or functional equivalent as depicted in column 8 of Table II B, application no. 2, and being depicted in the same respective line as said B0161, and preferably the activity is increased non-targeted.

Accordingly, in one embodiment, the molecule which activity is to be increased in the process of the invention is the gene product with an activity as a “serine protease”, preferably it is the molecule of section (a) or (b) of this paragraph.

In particular, it was observed that in plants, especially in Arabidopsis thaliana, increasing or generating the activity of a gene product non-targeted with the activity of a “serine protease”, preferably being encoded by a gene comprising the nucleic acid sequence SEQ ID NO. 7081, preferably the coding region thereof, conferred the production of or the increase in proline compared with the wild type control.

The nucleic acid sequence of B0221 from Escherichia coli, e.g. as shown in the respective line in column 5 of Table I, application no. 2, has been published in Saccharomyces cerevisiae Goffeau et al., Science 274 (5287), 546 (1996), Escherichia coli Blattner et al., Science 277 (5331), 1453 (1997)). And the activity of the gene product thereof is the activity of acyl-CoA dehydrogenase.

Accordingly, in one embodiment, the process of the present invention for producing arginine in a non-human organism, like a microorganism or a plant or a part thereof, comprises increasing or generating the activity of a gene product with the activity of a gene product conferring the activity of “acyl-CoA dehydrogenase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0221, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0221, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0221, or a functional equivalent or a homolog thereof as depicted in column 8 of Table II, application no. 2, preferably a homolog or functional equivalent as depicted in column 8 of Table II B, application no. 2, and being depicted in the same respective line as said B0221, and preferably the activity is increased non-targeted.

Accordingly, in one embodiment, the molecule which activity is to be increased in the process of the invention is the gene product with an activity as a “acyl-CoA dehydrogenase”, preferably it is the molecule of section (a) or (b) of this paragraph.

In particular, it was observed that in plants, especially in Arabidopsis thaliana, increasing or generating the activity of a gene product non-targeted with the activity of a “acyl-CoA dehydrogenase”, preferably being encoded by a gene comprising the nucleic acid sequence SEQ ID NO. 35590, preferably the coding region thereof, conferred the production of or the increase in arginine compared with the wild type control.

The nucleic acid sequence of B0221 from Escherichia coli, e.g. as shown in the respective line in column 5 of Table I, application no. 2, has been published in Blattner et al., Science 277 (5331), 1453 (1997)). And the activity of the gene product thereof is the activity of acyl-CoA dehydrogenase.

Accordingly, in one embodiment, the process of the present invention for producing glutamate in a non-human organism, like a microorganism or a plant or a part thereof, comprises increasing or generating the activity of a gene product with the activity of a gene product conferring the activity of “acyl-CoA dehydrogenase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0221, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0221, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0221, or a functional equivalent or a homolog thereof as depicted in column 8 of Table II, application no. 2, preferably a homolog or functional equivalent as depicted in column 8 of Table II B, application no. 2, and being depicted in the same respective line as said B0221, and preferably the activity is increased non-targeted.

Accordingly, in one embodiment, the molecule which activity is to be increased in the process of the invention is the gene product with an activity as a “acyl-CoA dehydrogenase”, preferably it is the molecule of section (a) or (b) of this paragraph.

In particular, it was observed that in plants, especially in Arabidopsis thaliana, increasing or generating the activity of a gene product non-targeted with the activity of a “acyl-CoA dehydrogenase”, preferably being encoded by a gene comprising the nucleic acid sequence SEQ ID NO. 35590, preferably the coding region thereof, conferred the production of or the increase in glutamate compared with the wild type control.

The nucleic acid sequence of B0344 from Escherichia coli, e.g. as shown in the respective line in column 5 of Table I, application no. 2, has been published in Blattner et al., Science 277 (5331), 1453 (1997)). And the activity of the gene product thereof is the activity of beta-galactosidase.

Accordingly, in one embodiment, the process of the present invention for producing proline in a non-human organism, like a microorganism or a plant or a part thereof, comprises increasing or generating the activity of a gene product with the activity of a gene product conferring the activity of “beta-galactosidase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0344, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0344, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0344, or a functional equivalent or a homolog thereof as depicted in column 8 of Table II, application no. 2, preferably a homolog or functional equivalent as depicted in column 8 of Table II B, application no. 2, and being depicted in the same respective line as said B0344, and preferably the activity is increased plastidic.

Accordingly, in one embodiment, the molecule which activity is to be increased in the process of the invention is the gene product with an activity as a “beta-galactosidase”, preferably it is the molecule of section (a) or (b) of this paragraph.

In particular, it was observed that in plants, especially in Arabidopsis thaliana, increasing or generating the activity of a gene product plastidic with the activity of a “beta-galactosidase”, preferably being encoded by a gene comprising the nucleic acid sequence SEQ ID NO. 35733, preferably the coding region thereof, conferred the production of or the increase in proline compared with the wild type control.

The nucleic acid sequence of B0449 from Escherichia coli, e.g. as shown in the respective line in column 5 of Table I, application no. 2, has been published in Blattner et al., Science 277 (5331), 1453 (1997)). And the activity of the gene product thereof is the activity of ATP-binding component of a transport system.

Accordingly, in one embodiment, the process of the present invention for producing glutamine in a non-human organism, like a microorganism or a plant or a part thereof, comprises increasing or generating the activity of a gene product with the activity of a gene product conferring the activity of “ATP-binding component of a transport system”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0449, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0449, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0449, or a functional equivalent or a homolog thereof as depicted in column 8 of Table II, application no. 2, preferably a homolog or functional equivalent as depicted in column 8 of Table II B, application no. 2, and being depicted in the same respective line as said B0449, and preferably the activity is increased non-targeted.

Accordingly, in one embodiment, the molecule which activity is to be increased in the process of the invention is the gene product with an activity as a “ATP-binding component of a transport system”, preferably it is the molecule of section (a) or (b) of this paragraph.

In particular, it was observed that in plants, especially in Arabidopsis thaliana, increasing or generating the activity of a gene product non-targeted with the activity of a “ATP-binding component of a transport system”, preferably being encoded by a gene comprising the nucleic acid sequence SEQ ID NO. 7333, preferably the coding region thereof, conferred the production of or the increase in glutamine compared with the wild type control.

The nucleic acid sequence of B0456 from Escherichia coli, e.g. as shown in the respective line in column 5 of Table I, application no. 2, has been published in Blattner et al., Science 277 (5331), 1453 (1997)). And the activity of the gene product thereof is the activity of b0456-protein.

Accordingly, in one embodiment, the process of the present invention for producing proline in a non-human organism, like a microorganism or a plant or a part thereof, comprises increasing or generating the activity of a gene product with the activity of a gene product conferring the activity of “b0456-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0456, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0456, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0456, or a functional equivalent or a homolog thereof as depicted in column 8 of Table II, application no. 2, preferably a homolog or functional equivalent as depicted in column 8 of Table II B, application no. 2, and being depicted in the same respective line as said B0456, and preferably the activity is increased non-targeted.
Accordingly, in one embodiment, the molecule which activity is to be increased in the process of the invention is the gene product with an activity as a “b0456-protein”, preferably it is the molecule of section (a) or (b) of this paragraph.

In particular, it was observed that in plants, especially in Arabidopsis thaliana, increasing or generating the activity of a gene product non-targeted with the activity of a “b0456-protein”, preferably being encoded by a gene comprising the nucleic acid sequence SEQ ID NO. 35875, preferably the coding region thereof, conferred the production of or the increase in proline compared with the wild type control.

The nucleic acid sequence of B0486 from Escherichia coli, e.g. as shown in the respective line in column 5 of Table I, application no. 2, has been published in Blattner et al., Science 277 (5331), 1453 (1997)). And the activity of the gene product thereof is the activity of membrane transport protein.

Accordingly, in one embodiment, the process of the present invention for producing arginine in a non-human organism, like a microorganism or a plant or a part thereof, comprises increasing or generating the activity of a gene product with the activity of a gene product conferring the activity of “membrane transport protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0486, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0486, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0486, or a functional equivalent or a homolog thereof as depicted in column 8 of Table II, application no. 2, preferably a homolog or functional equivalent as depicted in column 8 of Table II B, application no. 2, and being depicted in the same respective line as said B0486, and preferably the activity is increased non-targeted.

Accordingly, in one embodiment, the molecule which activity is to be increased in the process of the invention is the gene product with an activity as a “membrane transport protein”, preferably it is the molecule of section (a) or (b) of this paragraph.

In particular, it was observed that in plants, especially in Arabidopsis thaliana, increasing or generating the activity of a gene product non-targeted with the activity of a “membrane transport protein”, preferably being encoded by a gene comprising the nucleic acid sequence SEQ ID NO. 7686, preferably the coding region thereof, conferred the production of or the increase in arginine compared with the wild type control.

The nucleic acid sequence of B0486 from Escherichia coli, e.g. as shown in the respective line in column 5 of Table I, application no. 2, has been published in Blattner et al., Science 277 (5331), 1453 (1997)). And the activity of the gene product thereof is the activity of membrane transport protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “membrane transport protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0486, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0486, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0486, or a functional equivalent or a homolog thereof as depicted in column 8 of Table II, application no. 2, preferably a homolog or functional equivalent as depicted in column 8 of Table II B, application no. 2, and being depicted in the same respective line as said B0486, and preferably the activity is increased non-targeted.

Accordingly, in one embodiment, the molecule which activity is to be increased in the process of the invention is the gene product with an activity as a “membrane transport protein”, preferably it is the molecule of section (a) or (b) of this paragraph.

In particular, it was observed that in plants, especially in Arabidopsis thaliana, increasing or generating the activity of a gene product non-targeted with the activity of a “membrane transport protein”, preferably being encoded by a gene comprising the nucleic acid sequence SEQ ID NO. 7686, preferably the coding region thereof, conferred the production of or the increase in glutamate compared with the wild type control.

The nucleic acid sequence of B0518 from Escherichia coli, e.g. as shown in the respective line in column 5 of Table I, application no. 2, has been published in Blattner et al., Science 277 (5331), 1453 (1997)). And the activity of the gene product thereof is the activity of b0518-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “b0518-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0518, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0518, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0518, or a functional equivalent or a homolog thereof as depicted in column 8 of Table II, application no. 2, preferably a homolog or functional equivalent as depicted in column 8 of Table II B, application no. 2, and being depicted in the same respective line as said B0518, and preferably the activity is increased non-targeted.

Accordingly, in one embodiment, the molecule which activity is to be increased in the process of the invention is the gene product with an activity as a “b0518-protein”, preferably it is the molecule of section (a) or (b) of this paragraph.

In particular, it was observed that in plants, especially in Arabidopsis thaliana, increasing or generating the activity of a gene product non-targeted with the activity of a “b0518-protein”, preferably being encoded by a gene comprising the nucleic acid sequence SEQ ID NO. 35936, preferably the coding region thereof, conferred the production of or the increase in glutamine compared with the wild type control.

The nucleic acid sequence of B0593 from Escherichia coli, e.g. as shown in the respective line in column 5 of Table I, application no. 2, has been published in Blattner et al., Science 277 (5331), 1453 (1997)). And the activity of the gene product thereof is the activity of isochorismate synthase.

Accordingly, in one embodiment, the process of the present invention for producing arginine in a non-human organism, like a microorganism or a plant or a part thereof, comprises increasing or generating the activity of a gene product with the activity of a gene product conferring the activity of “isochorismate synthase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0593, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0593, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0593, or a functional equivalent or a homolog thereof as depicted in column 8 of Table II, application no. 2, preferably a homolog or functional equivalent as depicted in column 8 of Table II B, application no. 2, and being depicted in the same respective line as said B0593, and preferably the activity is increased plastidic.

Accordingly, in one embodiment, the molecule which activity is to be increased in the process of the invention is the gene product with an activity as a “isochorismate synthase”, preferably it is the molecule of section (a) or (b) of this paragraph.

In particular, it was observed that in plants, especially in Arabidopsis thaliana, increasing or generating the activity of a gene product plastidic with the activity of a “isochorismate synthase”, preferably being encoded by a gene comprising the nucleic acid sequence SEQ ID NO. 35967, preferably the coding region thereof, conferred the production of or the increase in arginine compared with the wild type control.

The nucleic acid sequence of B0593 from Escherichia coli, e.g. as shown in the respective line in column 5 of Table I, application no. 2, has been published in Blattner et al., Science 277 (5331), 1453 (1997)). And the activity of the gene product thereof is the activity of isochorismate synthase.

Accordingly, in one embodiment, the process of the present invention for producing glutamate in a non-human organism, like a microorganism or a plant or a part thereof, comprises increasing or generating the activity of a gene product with the activity of a gene product conferring the activity of “isochorismate synthase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0593, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0593, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0593, or a functional equivalent or a homolog thereof as depicted in column 8 of Table II, application no. 2, preferably a homolog or functional equivalent as depicted in column 8 of Table II B, application no. 2, and being depicted in the same respective line as said B0593, and preferably the activity is increased plastidic.

Accordingly, in one embodiment, the molecule which activity is to be increased in the process of the invention is the gene product with an activity as a “isochorismate synthase”, preferably it is the molecule of section (a) or (b) of this paragraph.

In particular, it was observed that in plants, especially in Arabidopsis thaliana, increasing or generating the activity of a gene product plastidic with the activity of a “isochorismate synthase”, preferably being encoded by a gene comprising the nucleic acid sequence SEQ ID NO. 35967, preferably the coding region thereof, conferred the production of or the increase in glutamate compared with the wild type control.

The nucleic acid sequence of B0593 from Escherichia coli, e.g. as shown in the respective line in column 5 of Table I, application no. 2, has been published in Blattner et al., Science 277 (5331), 1453 (1997)). And the activity of the gene product thereof is the activity of isochorismate synthase.

Accordingly, in one embodiment, the process of the present invention for producing glutamine in a non-human organism, like a microorganism or a plant or a part thereof, comprises increasing or generating the activity of a gene product with the activity of a gene product conferring the activity of “isochorismate synthase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0593, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0593, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0593, or a functional equivalent or a homolog thereof as depicted in column 8 of Table II, application no. 2, preferably a homolog or functional equivalent as depicted in column 8 of Table II B, application no. 2, and being depicted in the same respective line as said B0593, and preferably the activity is increased plastidic.

Accordingly, in one embodiment, the molecule which activity is to be increased in the process of the invention is the gene product with an activity as a “isochorismate synthase”, preferably it is the molecule of section (a) or (b) of this paragraph.

In particular, it was observed that in plants, especially in Arabidopsis thaliana, increasing or generating the activity of a gene product plastidic with the activity of a “isochorismate synthase”, preferably being encoded by a gene comprising the nucleic acid sequence SEQ ID NO. 35967, preferably the coding region thereof, conferred the production of or the increase in glutamine compared with the wild type control.

The nucleic acid sequence of B0593 from Escherichia coli, e.g. as shown in the respective line in column 5 of Table I, application no. 2, has been published in Blattner et al., Science 277 (5331), 1453 (1997)). And the activity of the gene product thereof is the activity of isochorismate synthase.

Accordingly, in one embodiment, the process of the present invention for producing proline in a non-human organism, like a microorganism or a plant or a part thereof, comprises increasing or generating the activity of a gene product with the activity of a gene product conferring the activity of “isochorismate synthase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0593, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0593, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0593, or a functional equivalent or a homolog thereof as depicted in column 8 of Table II, application no. 2, preferably a homolog or functional equivalent as depicted in column 8 of Table II B, application no. 2, and being depicted in the same respective line as said B0593, and preferably the activity is increased plastidic.

Accordingly, in one embodiment, the molecule which activity is to be increased in the process of the invention is the gene product with an activity as a “isochorismate synthase”, preferably it is the molecule of section (a) or (b) of this paragraph.

In particular, it was observed that in plants, especially in Arabidopsis thaliana, increasing or generating the activity of a gene product plastidic with the activity of a “isochorismate synthase”, preferably being encoded by a gene comprising the nucleic acid sequence SEQ ID NO. 35967, preferably the coding region thereof, conferred the production of or the increase in proline compared with the wild type control.

The nucleic acid sequence of B0752 from Escherichia coli, e.g. as shown in the respective line in column 5 of Table I, application no. 2, has been published in Blattner et al., Science 277 (5331), 1453 (1997)). And the activity of the gene product thereof is the activity of zinc transporter.

Accordingly, in one embodiment, the process of the present invention for producing glutamine in a non-human organism, like a microorganism or a plant or a part thereof, comprises increasing or generating the activity of a gene product with the activity of a gene product conferring the activity of “zinc transporter”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0752, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0752, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0752, or a functional equivalent or a homolog thereof as depicted in column 8 of Table II, application no. 2, preferably a homolog or functional equivalent as depicted in column 8 of Table II B, application no. 2, and being depicted in the same respective line as said B0752, and preferably the activity is increased non-targeted.

Accordingly, in one embodiment, the molecule which activity is to be increased in the process of the invention is the gene product with an activity as a “zinc transporter”, preferably it is the molecule of section (a) or (b) of this paragraph.

In particular, it was observed that in plants, especially in Arabidopsis thaliana, increasing or generating the activity of a gene product non-targeted with the activity of a “zinc transporter”, preferably being encoded by a gene comprising the nucleic acid sequence SEQ ID NO. 36114, preferably the coding region thereof, conferred the production of or the increase in glutamine compared with the wild type control.

The nucleic acid sequence of B0828 from Escherichia coli, e.g. as shown in the respective line in column 5 of Table I, application no. 2, has been published in Blattner et al., Science 277 (5331), 1453 (1997)). And the activity of the gene product thereof is the activity of asparaginase.

Accordingly, in one embodiment, the process of the present invention for producing glutamine in a non-human organism, like a microorganism or a plant or a part thereof, comprises increasing or generating the activity of a gene product with the activity of a gene product conferring the activity of “asparaginase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0828, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0828, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0828, or a functional equivalent or a homolog thereof as depicted in column 8 of Table II, application no. 2, preferably a homolog or functional equivalent as depicted in column 8 of Table II B, application no. 2, and being depicted in the same respective line as said B0828, and preferably the activity is increased non-targeted.

Accordingly, in one embodiment, the molecule which activity is to be increased in the process of the invention is the gene product with an activity as a “asparaginase”, preferably it is the molecule of section (a) or (b) of this paragraph.

In particular, it was observed that in plants, especially in Arabidopsis thaliana, increasing or generating the activity of a gene product non-targeted with the activity of a “asparaginase”, preferably being encoded by a gene comprising the nucleic acid sequence SEQ ID NO. 36299, preferably the coding region thereof, conferred the production of or the increase in glutamine compared with the wild type control.

The nucleic acid sequence of B0885 from Escherichia coli, e.g. as shown in the respective line in column 5 of Table I, application no. 2, has been published in Blattner et al., Science 277 (5331), 1453 (1997)). And the activity of the gene product thereof is the activity of Leucyl/phenylalanyl-tRNA-protein transferase.

Accordingly, in one embodiment, the process of the present invention for producing glutamine in a non-human organism, like a microorganism or a plant or a part thereof, comprises increasing or generating the activity of a gene product with the activity of a gene product conferring the activity of “Leucyl/phenylalanyl-tRNA-protein transferase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0885, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0885, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0885, or a functional equivalent or a homolog thereof as depicted in column 8 of Table II, application no. 2, preferably a homolog or functional equivalent as depicted in column 8 of Table II B, application no. 2, and being depicted in the same respective line as said B0885, and preferably the activity is increased non-targeted.

Accordingly, in one embodiment, the molecule which activity is to be increased in the process of the invention is the gene product with an activity as a “Leucyl/phenylalanyl-tRNA-protein transferase”, preferably it is the molecule of section (a) or (b) of this paragraph.

In particular, it was observed that in plants, especially in Arabidopsis thaliana, increasing or generating the activity of a gene product non-targeted with the activity of a “Leucyl/phenylalanyl-tRNA-protein transferase”, preferably being encoded by a gene comprising the nucleic acid sequence SEQ ID NO. 36489, preferably the coding region thereof, conferred the production of or the increase in glutamine compared with the wild type control.

The nucleic acid sequence of B0885 from Escherichia coli, e.g. as shown in the respective line in column 5 of Table I, application no. 2, has been published in Blattner et al., Science 277 (5331), 1453 (1997)). And the activity of the gene product thereof is the activity of Leucyl/phenylalanyl-tRNA-protein transferase.

Accordingly, in one embodiment, the process of the present invention for producing proline in a non-human organism, like a microorganism or a plant or a part thereof, comprises increasing or generating the activity of a gene product with the activity of a gene product conferring the activity of “Leucyl/phenylalanyl-tRNA-protein transferase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0885, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0885, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0885, or a functional equivalent or a homolog thereof as depicted in column 8 of Table II, application no. 2, preferably a homolog or functional equivalent as depicted in column 8 of Table II B, application no. 2, and being depicted in the same respective line as said B0885, and preferably the activity is increased non-targeted.

Accordingly, in one embodiment, the molecule which activity is to be increased in the process of the invention is the gene product with an activity as a “Leucyl/phenylalanyl-tRNA-protein transferase”, preferably it is the molecule of section (a) or (b) of this paragraph.

In particular, it was observed that in plants, especially in Arabidopsis thaliana, increasing or generating the activity of a gene product non-targeted with the activity of a “Leucyl/phenylalanyl-tRNA-protein transferase”, preferably being encoded by a gene comprising the nucleic acid sequence SEQ ID NO. 36489, preferably the coding region thereof, conferred the production of or the increase in proline compared with the wild type control.

The nucleic acid sequence of B0898 from Escherichia coli, e.g. as shown in the respective line in column 5 of Table I, application no. 2, has been published in Blattner et al., Science 277 (5331), 1453 (1997)). And the activity of the gene product thereof is the activity of major facilitator superfamily transporter protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “major facilitator superfamily transporter protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0898, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0898, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0898, or a functional equivalent or a homolog thereof as depicted in column 8 of Table II, application no. 2, preferably a homolog or functional equivalent as depicted in column 8 of Table II B, application no. 2, and being depicted in the same respective line as said B0898, and preferably the activity is increased non-targeted.

Accordingly, in one embodiment, the molecule which activity is to be increased in the process of the invention is the gene product with an activity as a “major facilitator superfamily transporter protein”, preferably it is the molecule of section (a) or (b) of this paragraph.

In particular, it was observed that in plants, especially in Arabidopsis thaliana, increasing or generating the activity of a gene product non-targeted with the activity of a “major facilitator superfamily transporter protein”, preferably being encoded by a gene comprising the nucleic acid sequence SEQ ID NO. 7917, preferably the coding region thereof, conferred the production of or the increase in glutamate compared with the wild type control.

The nucleic acid sequence of B0898 from Escherichia coli, e.g. as shown in the respective line in column 5 of Table I, application no. 2, has been published in Blattner et al., Science 277 (5331), 1453 (1997)). And the activity of the gene product thereof is the activity of major facilitator superfamily transporter protein.

Accordingly, in one embodiment, the process of the present invention for producing proline in a non-human organism, like a microorganism or a plant or a part thereof, comprises increasing or generating the activity of a gene product with the activity of a gene product conferring the activity of “major facilitator superfamily transporter protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0898, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0898, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0898, or a functional equivalent or a homolog thereof as depicted in column 8 of Table II, application no. 2, preferably a homolog or functional equivalent as depicted in column 8 of Table II B, application no. 2, and being depicted in the same respective line as said B0898, and preferably the activity is increased non-targeted.

Accordingly, in one embodiment, the molecule which activity is to be increased in the process of the invention is the gene product with an activity as a “major facilitator superfamily transporter protein”, preferably it is the molecule of section (a) or (b) of this paragraph.

In particular, it was observed that in plants, especially in Arabidopsis thaliana, increasing or generating the activity of a gene product non-targeted with the activity of a “major facilitator superfamily transporter protein”, preferably being encoded by a gene comprising the nucleic acid sequence SEQ ID NO. 7917, preferably the coding region thereof, conferred the production of or the increase in proline compared with the wild type control.

The nucleic acid sequence of B0962 from Escherichia coli, e.g. as shown in the respective line in column 5 of Table I, application no. 2, has been published in Blattner et al., Science 277 (5331), 1453 (1997)). And the activity of the gene product thereof is the activity of DNA helicase IV.

Accordingly, in one embodiment, the process of the present invention for producing proline in a non-human organism, like a microorganism or a plant or a part thereof, comprises increasing or generating the activity of a gene product with the activity of a gene product conferring the activity of “DNA helicase IV”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0962, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0962, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0962, or a functional equivalent or a homolog thereof as depicted in column 8 of Table II, application no. 2, preferably a homolog or functional equivalent as depicted in column 8 of Table II B, application no. 2, and being depicted in the same respective line as said B0962, and preferably the activity is increased non-targeted.

Accordingly, in one embodiment, the molecule which activity is to be increased in the process of the invention is the gene product with an activity as a “DNA helicase IV”, preferably it is the molecule of section (a) or (b) of this paragraph.

In particular, it was observed that in plants, especially in Arabidopsis thaliana, increasing or generating the activity of a gene product non-targeted with the activity of a “DNA helicase IV”, preferably being encoded by a gene comprising the nucleic acid sequence SEQ ID NO. 36623, preferably the coding region thereof, conferred the production of or the increase in proline compared with the wild type control.

The nucleic acid sequence of B0963 from Escherichia coli, e.g. as shown in the respective line in column 5 of Table I, application no. 2, has been published in Blattner et al., Science 277 (5331), 1453 (1997)). And the activity of the gene product thereof is the activity of methylglyoxal synthase.

Accordingly, in one embodiment, the process of the present invention for producing proline in a non-human organism, like a microorganism or a plant or a part thereof, comprises increasing or generating the activity of a gene product with the activity of a gene product conferring the activity of “methylglyoxal synthase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0963, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0963, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0963, or a functional equivalent or a homolog thereof as depicted in column 8 of Table II, application no. 2, preferably a homolog or functional equivalent as depicted in column 8 of Table II B, application no. 2, and being depicted in the same respective line as said B0963, and preferably the activity is increased non-targeted.

Accordingly, in one embodiment, the molecule which activity is to be increased in the process of the invention is the gene product with an activity as a “methylglyoxal synthase”, preferably it is the molecule of section (a) or (b) of this paragraph.

In particular, it was observed that in plants, especially in Arabidopsis thaliana, increasing or generating the activity of a gene product non-targeted with the activity of a “methylglyoxal synthase”, preferably being encoded by a gene comprising the nucleic acid sequence SEQ ID NO. 36670, preferably the coding region thereof, conferred the production of or the increase in proline compared with the wild type control.

The nucleic acid sequence of B0980 from Escherichia coli, e.g. as shown in the respective line in column 5 of Table I, application no. 2, has been published in Blattner et al., Science 277 (5331), 1453 (1997)). And the activity of the gene product thereof is the activity of phosphoanhydride phosphorylase.

Accordingly, in one embodiment, the process of the present invention for producing proline in a non-human organism, like a microorganism or a plant or a part thereof, comprises increasing or generating the activity of a gene product with the activity of a gene product conferring the activity of “phosphoanhydride phosphorylase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B0980, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B0980, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B0980, or a functional equivalent or a homolog thereof as depicted in column 8 of Table II, application no. 2, preferably a homolog or functional equivalent as depicted in column 8 of Table II B, application no. 2, and being depicted in the same respective line as said B0980, and preferably the activity is increased non-targeted.

Accordingly, in one embodiment, the molecule which activity is to be increased in the process of the invention is the gene product with an activity as a “phosphoanhydride phosphorylase”, preferably it is the molecule of section (a) or (b) of this paragraph.

In particular, it was observed that in plants, especially in Arabidopsis thaliana, increasing or generating the activity of a gene product non-targeted with the activity of a “phosphoanhydride phosphorylase”, preferably being encoded by a gene comprising the nucleic acid sequence SEQ ID NO. 36809, preferably the coding region thereof, conferred the production of or the increase in proline compared with the wild type control.

The nucleic acid sequence of B1003 from Escherichia coli, e.g. as shown in the respective line in column 5 of Table I, application no. 2, has been published in Blattner et al., Science 277 (5331), 1453 (1997)). And the activity of the gene product thereof is the activity of b1003-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “b1003-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1003, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1003, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1003, or a functional equivalent or a homolog thereof as depicted in column 8 of Table II, application no. 2, preferably a homolog or functional equivalent as depicted in column 8 of Table II B, application no. 2, and being depicted in the same respective line as said B1003, and preferably the activity is increased non-targeted.

Accordingly, in one embodiment, the molecule which activity is to be increased in the process of the invention is the gene product with an activity as a “b1003-protein”, preferably it is the molecule of section (a) or (b) of this paragraph.

In particular, it was observed that in plants, especially in Arabidopsis thaliana, increasing or generating the activity of a gene product non-targeted with the activity of a “b1003-protein”, preferably being encoded by a gene comprising the nucleic acid sequence SEQ ID NO. 7941, preferably the coding region thereof, conferred the production of or the increase in glutamine compared with the wild type control.

The nucleic acid sequence of B1003 from Escherichia coli, e.g. as shown in the respective line in column 5 of Table I, application no. 2, has been published in Blattner et al., Science 277 (5331), 1453 (1997)). And the activity of the gene product thereof is the activity of b1003-protein.

Accordingly, in one embodiment, the process of the present invention for producing proline in a non-human organism, like a microorganism or a plant or a part thereof, comprises increasing or generating the activity of a gene product with the activity of a gene product conferring the activity of “b1003-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1003, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1003, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1003, or a functional equivalent or a homolog thereof as depicted in column 8 of Table II, application no. 2, preferably a homolog or functional equivalent as depicted in column 8 of Table II B, application no. 2, and being depicted in the same respective line as said B1003, and preferably the activity is increased non-targeted.

Accordingly, in one embodiment, the molecule which activity is to be increased in the process of the invention is the gene product with an activity as a “b1003-protein”, preferably it is the molecule of section (a) or (b) of this paragraph.

In particular, it was observed that in plants, especially in Arabidopsis thaliana, increasing or generating the activity of a gene product non-targeted with the activity of a “b1003-protein”, preferably being encoded by a gene comprising the nucleic acid sequence SEQ ID NO. 7941, preferably the coding region thereof, conferred the production of or the increase in proline compared with the wild type control.

The nucleic acid sequence of B1023 from Escherichia coli, e.g. as shown in the respective line in column 5 of Table I, application no. 2, has been published in Blattner et al., Science 277 (5331), 1453 (1997)). And the activity of the gene product thereof is the activity of lipoprotein precursor.

Accordingly, in one embodiment, the process of the present invention for producing arginine in a non-human organism, like a microorganism or a plant or a part thereof, comprises increasing or generating the activity of a gene product with the activity of a gene product conferring the activity of “lipoprotein precursor”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1023, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1023, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1023, or a functional equivalent or a homolog thereof as depicted in column 8 of Table II, application no. 2, preferably a homolog or functional equivalent as depicted in column 8 of Table II B, application no. 2, and being depicted in the same respective line as said B1023, and preferably the activity is increased non-targeted,.

Accordingly, in one embodiment, the molecule which activity is to be increased in the process of the invention is the gene product with an activity as a “lipoprotein precursor”, preferably it is the molecule of section (a) or (b) of this paragraph.

In particular, it was observed that in plants, especially in Arabidopsis thaliana, increasing or generating the activity of a gene product non-targeted with the activity of a “lipoprotein precursor”, preferably being encoded by a gene comprising the nucleic acid sequence SEQ ID NO. 36880, preferably the coding region thereof, conferred the production of or the increase in arginine compared with the wild type control.

The nucleic acid sequence of B1024 from Escherichia coli, e.g. as shown in the respective line in column 5 of Table I, application no. 2, has been published in Blattner et al., Science 277 (5331), 1453 (1997)). And the activity of the gene product thereof is the activity of b1024-protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “b1024-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1024, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1024, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1024, or a functional equivalent or a homolog thereof as depicted in column 8 of Table II, application no. 2, preferably a homolog or functional equivalent as depicted in column 8 of Table II B, application no. 2, and being depicted in the same respective line as said B1024, and preferably the activity is increased non-targeted.

Accordingly, in one embodiment, the molecule which activity is to be increased in the process of the invention is the gene product with an activity as a “b1024-protein”, preferably it is the molecule of section (a) or (b) of this paragraph.

In particular, it was observed that in plants, especially in Arabidopsis thaliana, increasing or generating the activity of a gene product non-targeted with the activity of a “b1024-protein”, preferably being encoded by a gene comprising the nucleic acid sequence SEQ ID NO. 36907, preferably the coding region thereof, conferred the production of or the increase in glutamate compared with the wild type control.

The nucleic acid sequence of B1108 from Escherichia coli, e.g. as shown in the respective line in column 5 of Table I, application no. 2, has been published in Blattner et al., Science 277 (5331), 1453 (1997)). And the activity of the gene product thereof is the activity of b1108-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “b1108-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1108, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1108, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1108, or a functional equivalent or a homolog thereof as depicted in column 8 of Table II, application no. 2, preferably a homolog or functional equivalent as depicted in column 8 of Table II B, application no. 2, and being depicted in the same respective line as said B1108, and preferably the activity is increased non-targeted.

Accordingly, in one embodiment, the molecule which activity is to be increased in the process of the invention is the gene product with an activity as a “b1108-protein”, preferably it is the molecule of section (a) or (b) of this paragraph.

In particular, it was observed that in plants, especially in Arabidopsis thaliana, increasing or generating the activity of a gene product non-targeted with the activity of a “b1108-protein”, preferably being encoded by a gene comprising the nucleic acid sequence SEQ ID NO. 36937, preferably the coding region thereof, conferred the production of or the increase in glutamine compared with the wild type control.

The nucleic acid sequence of B1108 from Escherichia coli, e.g. as shown in the respective line in column 5 of Table I, application no. 2, has been published in Blattner et al., Science 277 (5331), 1453 (1997)). And the activity of the gene product thereof is the activity of b1108-protein.

Accordingly, in one embodiment, the process of the present invention for producing proline in a non-human organism, like a microorganism or a plant or a part thereof, comprises increasing or generating the activity of a gene product with the activity of a gene product conferring the activity of “b1108-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1108, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1108, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1108, or a functional equivalent or a homolog thereof as depicted in column 8 of Table II, application no. 2, preferably a homolog or functional equivalent as depicted in column 8 of Table II B, application no. 2, and being depicted in the same respective line as said B1108, and preferably the activity is increased non-targeted.

Accordingly, in one embodiment, the molecule which activity is to be increased in the process of the invention is the gene product with an activity as a “b1108-protein”, preferably it is the molecule of section (a) or (b) of this paragraph.

In particular, it was observed that in plants, especially in Arabidopsis thaliana, increasing or generating the activity of a gene product non-targeted with the activity of a “b1108-protein”, preferably being encoded by a gene comprising the nucleic acid sequence SEQ ID NO. 36937, preferably the coding region thereof, conferred the production of or the increase in proline compared with the wild type control.

The nucleic acid sequence of B1136 from Escherichia coli, e.g. as shown in the respective line in column 5 of Table I, application no. 2, has been published in Blattner et al., Science 277 (5331), 1453 (1997)). And the activity of the gene product thereof is the activity of isocitrate dehydrogenase.

Accordingly, in one embodiment, the process of the present invention for producing proline in a non-human organism, like a microorganism or a plant or a part thereof, comprises increasing or generating the activity of a gene product with the activity of a gene product conferring the activity of “isocitrate dehydrogenase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1136, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1136, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1136, or a functional equivalent or a homolog thereof as depicted in column 8 of Table II, application no. 2, preferably a homolog or functional equivalent as depicted in column 8 of Table II B, application no. 2, and being depicted in the same respective line as said B1136, and preferably the activity is increased plastidic.

Accordingly, in one embodiment, the molecule which activity is to be increased in the process of the invention is the gene product with an activity as a “isocitrate dehydrogenase”, preferably it is the molecule of section (a) or (b) of this paragraph.

In particular, it was observed that in plants, especially in Arabidopsis thaliana, increasing or generating the activity of a gene product plastidic with the activity of a “isocitrate dehydrogenase”, preferably being encoded by a gene comprising the nucleic acid sequence SEQ ID NO. 36971, preferably the coding region thereof, conferred the production of or the increase in proline compared with the wild type control.

The nucleic acid sequence of B1137 from Escherichia coli, e.g. as shown in the respective line in column 5 of Table I, application no. 2, has been published in Blattner et al., Science 277 (5331), 1453 (1997)). And the activity of the gene product thereof is the activity of b1137-protein.

Accordingly, in one embodiment, the process of the present invention for producing arginine in a non-human organism, like a microorganism or a plant or a part thereof, comprises increasing or generating the activity of a gene product with the activity of a gene product conferring the activity of “b1137-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1137, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1137, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1137, or a functional equivalent or a homolog thereof as depicted in column 8 of Table II, application no. 2, preferably a homolog or functional equivalent as depicted in column 8 of Table II B, application no. 2, and being depicted in the same respective line as said B1137, and preferably the activity is increased non-targeted,.

Accordingly, in one embodiment, the molecule which activity is to be increased in the process of the invention is the gene product with an activity as a “b1137-protein”, preferably it is the molecule of section (a) or (b) of this paragraph.

In particular, it was observed that in plants, especially in Arabidopsis thaliana, increasing or generating the activity of a gene product non-targeted with the activity of a “b1137-protein”, preferably being encoded by a gene comprising the nucleic acid sequence SEQ ID NO. 37390, preferably the coding region thereof, conferred the production of or the increase in arginine compared with the wild type control.

The nucleic acid sequence of B1137 from Escherichia coli, e.g. as shown in the respective line in column 5 of Table I, application no. 2, has been published in Blattner et al., Science 277 (5331), 1453 (1997)). And the activity of the gene product thereof is the activity of b1137-protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “b1137-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1137, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1137, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1137, or a functional equivalent or a homolog thereof as depicted in column 8 of Table II, application no. 2, preferably a homolog or functional equivalent as depicted in column 8 of Table II B, application no. 2, and being depicted in the same respective line as said B1137, and preferably the activity is increased non-targeted.

Accordingly, in one embodiment, the molecule which activity is to be increased in the process of the invention is the gene product with an activity as a “b1137-protein”, preferably it is the molecule of section (a) or (b) of this paragraph.

In particular, it was observed that in plants, especially in Arabidopsis thaliana, increasing or generating the activity of a gene product non-targeted with the activity of a “b1137-protein”, preferably being encoded by a gene comprising the nucleic acid sequence SEQ ID NO. 37390, preferably the coding region thereof, conferred the production of or the increase in glutamate compared with the wild type control.

The nucleic acid sequence of B1137 from Escherichia coli, e.g. as shown in the respective line in column 5 of Table I, application no. 2, has been published in Blattner et al., Science 277 (5331), 1453 (1997)). And the activity of the gene product thereof is the activity of b1137-protein.

Accordingly, in one embodiment, the process of the present invention for producing proline in a non-human organism, like a microorganism or a plant or a part thereof, comprises increasing or generating the activity of a gene product with the activity of a gene product conferring the activity of “b1137-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1137, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1137, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1137, or a functional equivalent or a homolog thereof as depicted in column 8 of Table II, application no. 2, preferably a homolog or functional equivalent as depicted in column 8 of Table II B, application no. 2, and being depicted in the same respective line as said B1137, and preferably the activity is increased non-targeted.

Accordingly, in one embodiment, the molecule which activity is to be increased in the process of the invention is the gene product with an activity as a “b1137-protein”, preferably it is the molecule of section (a) or (b) of this paragraph.

In particular, it was observed that in plants, especially in Arabidopsis thaliana, increasing or generating the activity of a gene product non-targeted with the activity of a “b1137-protein”, preferably being encoded by a gene comprising the nucleic acid sequence SEQ ID NO. 37390, preferably the coding region thereof, conferred the production of or the increase in proline compared with the wild type control.

The nucleic acid sequence of B1163 from Escherichia coli, e.g. as shown in the respective line in column 5 of Table I, application no. 2, has been published in Blattner et al., Science 277 (5331), 1453 (1997)). And the activity of the gene product thereof is the activity of b1163-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “b1163-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1163, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1163, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1163, or a functional equivalent or a homolog thereof as depicted in column 8 of Table II, application no. 2, preferably a homolog or functional equivalent as depicted in column 8 of Table II B, application no. 2, and being depicted in the same respective line as said B1163, and preferably the activity is increased non-targeted.

Accordingly, in one embodiment, the molecule which activity is to be increased in the process of the invention is the gene product with an activity as a “b1163-protein”, preferably it is the molecule of section (a) or (b) of this paragraph.

In particular, it was observed that in plants, especially in Arabidopsis thaliana, increasing or generating the activity of a gene product non-targeted with the activity of a “b1163-protein”, preferably being encoded by a gene comprising the nucleic acid sequence SEQ ID NO. 37394, preferably the coding region thereof, conferred the production of or the increase in glutamine compared with the wild type control.

The nucleic acid sequence of B1186 from Escherichia coli K12, e.g. as shown in the respective line in column 5 of Table I, application no. 2, has been published in Blattner et al., Science 277 (5331), 1453 (1997)). And the activity of the gene product thereof is the activity of sodium/proton antiporter.

Accordingly, in one embodiment, the process of the present invention for producing proline in a non-human organism, like a microorganism or a plant or a part thereof, comprises increasing or generating the activity of a gene product with the activity of a gene product conferring the activity of “sodium/proton antiporter”, especially from Escherichia coli K12 or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1186, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1186, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1186, or a functional equivalent or a homolog thereof as depicted in column 8 of Table II, application no. 2, preferably a homolog or functional equivalent as depicted in column 8 of Table II B, application no. 2, and being depicted in the same respective line as said B1186, and preferably the activity is increased non-targeted.

Accordingly, in one embodiment, the molecule which activity is to be increased in the process of the invention is the gene product with an activity as a “sodium/proton antiporter”, preferably it is the molecule of section (a) or (b) of this paragraph.

In particular, it was observed that in plants, especially in Arabidopsis thaliana, increasing or generating the activity of a gene product non-targeted with the activity of a “sodium/proton antiporter”, preferably being encoded by a gene comprising the nucleic acid sequence SEQ ID NO. 37400, preferably the coding region thereof, conferred the production of or the increase in proline compared with the wild type control.

The nucleic acid sequence of B1255 from Escherichia coli, e.g. as shown in the respective line in column 5 of Table I, application no. 2, has been published in Blattner et al., Science 277 (5331), 1453 (1997)). And the activity of the gene product thereof is the activity of membrane protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “membrane protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1255, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1255, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1255, or a functional equivalent or a homolog thereof as depicted in column 8 of Table II, application no. 2, preferably a homolog or functional equivalent as depicted in column 8 of Table II B, application no. 2, and being depicted in the same respective line as said B1255, and preferably the activity is increased non-targeted,.

Accordingly, in one embodiment, the molecule which activity is to be increased in the process of the invention is the gene product with an activity as a “membrane protein”, preferably it is the molecule of section (a) or (b) of this paragraph.

In particular, it was observed that in plants, especially in Arabidopsis thaliana, increasing or generating the activity of a gene product non-targeted with the activity of a “membrane protein”, preferably being encoded by a gene comprising the nucleic acid sequence SEQ ID NO. 37483, preferably the coding region thereof, conferred the production of or the increase in glutamine compared with the wild type control.

The nucleic acid sequence of B1255 from Escherichia coli, e.g. as shown in the respective line in column 5 of Table I, application no. 2, has been published Blattner et al., Science 277 (5331), 1453 (1997)). And the activity of the gene product thereof is the activity of membrane protein.

Accordingly, in one embodiment, the process of the present invention for producing proline in a non-human organism, like a microorganism or a plant or a part thereof, comprises increasing or generating the activity of a gene product with the activity of a gene product conferring the activity of “membrane protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1255, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1255, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1255, or a functional equivalent or a homolog thereof as depicted in column 8 of Table II, application no. 2, preferably a homolog or functional equivalent as depicted in column 8 of Table II B, application no. 2, and being depicted in the same respective line as said B1255, and preferably the activity is increased non-targeted.

Accordingly, in one embodiment, the molecule which activity is to be increased in the process of the invention is the gene product with an activity as a “membrane protein”, preferably it is the molecule of section (a) or (b) of this paragraph.

In particular, it was observed that in plants, especially in Arabidopsis thaliana, increasing or generating the activity of a gene product non-targeted with the activity of a “membrane protein”, preferably being encoded by a gene comprising the nucleic acid sequence SEQ ID NO. 37483, preferably the coding region thereof, conferred the production of or the increase in proline compared with the wild type control.

The nucleic acid sequence of B1259 from Escherichia coli, e.g. as shown in the respective line in column 5 of Table I, application no. 2, has been published in Blattner et al., Science 277 (5331), 1453 (1997)). And the activity of the gene product thereof is the activity of b1259-protein.

Accordingly, in one embodiment, the process of the present invention for producing arginine in a non-human organism, like a microorganism or a plant or a part thereof, comprises increasing or generating the activity of a gene product with the activity of a gene product conferring the activity of “b1259-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1259, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1259, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1259, or a functional equivalent or a homolog thereof as depicted in column 8 of Table II, application no. 2, preferably a homolog or functional equivalent as depicted in column 8 of Table II B, application no. 2, and being depicted in the same respective line as said B1259, and preferably the activity is increased non-targeted.

Accordingly, in one embodiment, the molecule which activity is to be increased in the process of the invention is the gene product with an activity as a “b1259-protein”, preferably it is the molecule of section (a) or (b) of this paragraph.

In particular, it was observed that in plants, especially in Arabidopsis thaliana, increasing or generating the activity of a gene product non-targeted with the activity of a “b1259-protein”, preferably being encoded by a gene comprising the nucleic acid sequence SEQ ID NO. 37503, preferably the coding region thereof, conferred the production of or the increase in arginine compared with the wild type control.

The nucleic acid sequence of B1259 from Escherichia coli, e.g. as shown in the respective line in column 5 of Table I, application no. 2, has been published in Blattner et al., Science 277 (5331), 1453 (1997)). And the activity of the gene product thereof is the activity of b1259-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “b1259-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1259, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1259, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1259, or a functional equivalent or a homolog thereof as depicted in column 8 of Table II, application no. 2, preferably a homolog or functional equivalent as depicted in column 8 of Table II B, application no. 2, and being depicted in the same respective line as said B1259, and preferably the activity is increased non-targeted.

Accordingly, in one embodiment, the molecule which activity is to be increased in the process of the invention is the gene product with an activity as a “b1259-protein”, preferably it is the molecule of section (a) or (b) of this paragraph.

In particular, it was observed that in plants, especially in Arabidopsis thaliana, increasing or generating the activity of a gene product non-targeted with the activity of a “b1259-protein”, preferably being encoded by a gene comprising the nucleic acid sequence SEQ ID NO. 37503, preferably the coding region thereof, conferred the production of or the increase in glutamine compared with the wild type control.

The nucleic acid sequence of B1263 from Escherichia coli, e.g. as shown in the respective line in column 5 of Table I, application no. 2, has been published in Blattner et al., Science 277 (5331), 1453 (1997)). And the activity of the gene product thereof is the activity of anthranilate synthase component II.

Accordingly, in one embodiment, the process of the present invention for producing glutamine in a non-human organism, like a microorganism or a plant or a part thereof, comprises increasing or generating the activity of a gene product with the activity of a gene product conferring the activity of “anthranilate synthase component II”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1263, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1263, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1263, or a functional equivalent or a homolog thereof as depicted in column 8 of Table II, application no. 2, preferably a homolog or functional equivalent as depicted in column 8 of Table II B, application no. 2, and being depicted in the same respective line as said B1263, and preferably the activity is increased plastidic.

Accordingly, in one embodiment, the molecule which activity is to be increased in the process of the invention is the gene product with an activity as a “anthranilate synthase component II”, preferably it is the molecule of section (a) or (b) of this paragraph.

In particular, it was observed that in plants, especially in Arabidopsis thaliana, increasing or generating the activity of a gene product plastidic with the activity of a “anthranilate synthase component II”, preferably being encoded by a gene comprising the nucleic acid sequence SEQ ID NO. 37539, preferably the coding region thereof, conferred the production of or the increase in glutamine compared with the wild type control.

The nucleic acid sequence of B1280 from Escherichia coli, e.g. as shown in the respective line in column 5 of Table I, application no. 2, has been published in Blattner et al., Science 277 (5331), 1453 (1997)). And the activity of the gene product thereof is the activity of b1280-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “b1280-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1280, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1280, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1280, or a functional equivalent or a homolog thereof as depicted in column 8 of Table II, application no. 2, preferably a homolog or functional equivalent as depicted in column 8 of Table II B, application no. 2, and being depicted in the same respective line as said B1280, and preferably the activity is increased non-targeted.

Accordingly, in one embodiment, the molecule which activity is to be increased in the process of the invention is the gene product with an activity as a “b1280-protein”, preferably it is the molecule of section (a) or (b) of this paragraph.

In particular, it was observed that in plants, especially in Arabidopsis thaliana, increasing or generating the activity of a gene product non-targeted with the activity of a “b1280-protein”, preferably being encoded by a gene comprising the nucleic acid sequence SEQ ID NO. 37573, preferably the coding region thereof, conferred the production of or the increase in glutamine compared with the wild type control.

The nucleic acid sequence of B1297 from Escherichia coli, e.g. as shown in the respective line in column 5 of Table I, application no. 2, has been published in Blattner et al., Science 277 (5331), 1453 (1997)). And the activity of the gene product thereof is the activity of glutamine synthetase.

Accordingly, in one embodiment, the process of the present invention for producing glutamine in a non-human organism, like a microorganism or a plant or a part thereof, comprises increasing or generating the activity of a gene product with the activity of a gene product conferring the activity of “glutamine synthetase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1297, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1297, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1297, or a functional equivalent or a homolog thereof as depicted in column 8 of Table II, application no. 2, preferably a homolog or functional equivalent as depicted in column 8 of Table II B, application no. 2, and being depicted in the same respective line as said B1297, and preferably the activity is increased plastidic.

Accordingly, in one embodiment, the molecule which activity is to be increased in the process of the invention is the gene product with an activity as a “glutamine synthetase”, preferably it is the molecule of section (a) or (b) of this paragraph.

In particular, it was observed that in plants, especially in Arabidopsis thaliana, increasing or generating the activity of a gene product plastidic with the activity of a “glutamine synthetase”, preferably being encoded by a gene comprising the nucleic acid sequence SEQ ID NO. 37658, preferably the coding region thereof, conferred the production of or the increase in glutamine compared with the wild type control.

The nucleic acid sequence of B1300 from Escherichia coli, e.g. as shown in the respective line in column 5 of Table I, application no. 2, has been published in Blattner et al., Science 277 (5331), 1453 (1997)). And the activity of the gene product thereof is the activity of aldehyde dehydrogenase.

Accordingly, in one embodiment, the process of the present invention for producing glutamate in a non-human organism, like a microorganism or a plant or a part thereof, comprises increasing or generating the activity of a gene product with the activity of a gene product conferring the activity of “aldehyde dehydrogenase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1300, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1300, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1300, or a functional equivalent or a homolog thereof as depicted in column 8 of Table II, application no. 2, preferably a homolog or functional equivalent as depicted in column 8 of Table II B, application no. 2, and being depicted in the same respective line as said B1300, and preferably the activity is increased non-targeted.

Accordingly, in one embodiment, the molecule which activity is to be increased in the process of the invention is the gene product with an activity as a “aldehyde dehydrogenase”, preferably it is the molecule of section (a) or (b) of this paragraph.

In particular, it was observed that in plants, especially in Arabidopsis thaliana, increasing or generating the activity of a gene product non-targeted with the activity of a “aldehyde dehydrogenase”, preferably being encoded by a gene comprising the nucleic acid sequence SEQ ID NO. 37807, preferably the coding region thereof, conferred the production of or the increase in glutamate compared with the wild type control.

The nucleic acid sequence of B1330 from Escherichia coli, e.g. as shown in the respective line in column 5 of Table I, application no. 2, has been published in Blattner et al., Science 277 (5331), 1453 (1997)). And the activity of the gene product thereof is the activity of b1330-protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “b1330-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1330, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1330, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1330, or a functional equivalent or a homolog thereof as depicted in column 8 of Table II, application no. 2, preferably a homolog or functional equivalent as depicted in column 8 of Table II B, application no. 2, and being depicted in the same respective line as said B1330, and preferably the activity is increased non-targeted.

Accordingly, in one embodiment, the molecule which activity is to be increased in the process of the invention is the gene product with an activity as a “b1330-protein”, preferably it is the molecule of section (a) or (b) of this paragraph.

In particular, it was observed that in plants, especially in Arabidopsis thaliana, increasing or generating the activity of a gene product non-targeted with the activity of a “b1330-protein”, preferably being encoded by a gene comprising the nucleic acid sequence SEQ ID NO. 38226, preferably the coding region thereof, conferred the production of or the increase in glutamate compared with the wild type control.

The nucleic acid sequence of B1330 from Escherichia coli, e.g. as shown in the respective line in column 5 of Table I, application no. 2, has been published in Blattner et al., Science 277 (5331), 1453 (1997)). And the activity of the gene product thereof is the activity of b1330-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “b1330-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1330, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1330, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1330, or a functional equivalent or a homolog thereof as depicted in column 8 of Table II, application no. 2, preferably a homolog or functional equivalent as depicted in column 8 of Table II B, application no. 2, and being depicted in the same respective line as said B1330, and preferably the activity is increased non-targeted.

Accordingly, in one embodiment, the molecule which activity is to be increased in the process of the invention is the gene product with an activity as a “b1330-protein”, preferably it is the molecule of section (a) or (b) of this paragraph.

In particular, it was observed that in plants, especially in Arabidopsis thaliana, increasing or generating the activity of a gene product non-targeted with the activity of a “b1330-protein”, preferably being encoded by a gene comprising the nucleic acid sequence SEQ ID NO. 38226, preferably the coding region thereof, conferred the production of or the increase in glutamine compared with the wild type control.

The nucleic acid sequence of B1431 from Escherichia coli, e.g. as shown in the respective line in column 5 of Table I, application no. 2, has been published in Blattner et al., Science 277 (5331), 1453 (1997)). And the activity of the gene product thereof is the activity of lipoprotein precursor.

Accordingly, in one embodiment, the process of the present invention for producing glutamate in a non-human organism, like a microorganism or a plant or a part thereof, comprises increasing or generating the activity of a gene product with the activity of a gene product conferring the activity of “lipoprotein precursor”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1431, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1431, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1431, or a functional equivalent or a homolog thereof as depicted in column 8 of Table II, application no. 2, preferably a homolog or functional equivalent as depicted in column 8 of Table II B, application no. 2, and being depicted in the same respective line as said B1431, and preferably the activity is increased plastidic.

Accordingly, in one embodiment, the molecule which activity is to be increased in the process of the invention is the gene product with an activity as a “lipoprotein precursor”, preferably it is the molecule of section (a) or (b) of this paragraph.

In particular, it was observed that in plants, especially in Arabidopsis thaliana, increasing or generating the activity of a gene product plastidic with the activity of a “lipoprotein precursor”, preferably being encoded by a gene comprising the nucleic acid sequence SEQ ID NO. 38266, preferably the coding region thereof, conferred the production of or the increase in glutamate compared with the wild type control.

The nucleic acid sequence of B1431 from Escherichia coli, e.g. as shown in the respective line in column 5 of Table I, application no. 2, has been published in Blattner et al., Science 277 (5331), 1453 (1997)). And the activity of the gene product thereof is the activity of lipoprotein precursor.

Accordingly, in one embodiment, the process of the present invention for producing glutamine in a non-human organism, like a microorganism or a plant or a part thereof, comprises increasing or generating the activity of a gene product with the activity of a gene product conferring the activity of “lipoprotein precursor”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1431, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1431, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1431, or a functional equivalent or a homolog thereof as depicted in column 8 of Table II, application no. 2, preferably a homolog or functional equivalent as depicted in column 8 of Table II B, application no. 2, and being depicted in the same respective line as said B1431, and preferably the activity is increased plastidic.

Accordingly, in one embodiment, the molecule which activity is to be increased in the process of the invention is the gene product with an activity as a “lipoprotein precursor”, preferably it is the molecule of section (a) or (b) of this paragraph.

In particular, it was observed that in plants, especially in Arabidopsis thaliana, increasing or generating the activity of a gene product plastidic with the activity of a “lipoprotein precursor”, preferably being encoded by a gene comprising the nucleic acid sequence SEQ ID NO. 38266, preferably the coding region thereof, conferred the production of or the increase in glutamine compared with the wild type control.

The nucleic acid sequence of B1445 from Escherichia coli, e.g. as shown in the respective line in column 5 of Table I, application no. 2, has been published in Blattner et al., Science 277 (5331), 1453 (1997)) And the activity of the gene product thereof is the activity of b1445-protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “b1445-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1445, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1445, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1445, or a functional equivalent or a homolog thereof as depicted in column 8 of Table II, application no. 2, preferably a homolog or functional equivalent as depicted in column 8 of Table II B, application no. 2, and being depicted in the same respective line as said B1445, and preferably the activity is increased non-targeted.

Accordingly, in one embodiment, the molecule which activity is to be increased in the process of the invention is the gene product with an activity as a “b1445-protein”, preferably it is the molecule of section (a) or (b) of this paragraph.

In particular, it was observed that in plants, especially in Arabidopsis thaliana, increasing or generating the activity of a gene product non-targeted with the activity of a “b1445-protein”, preferably being encoded by a gene comprising the nucleic acid sequence SEQ ID NO. 38289, preferably the coding region thereof, conferred the production of or the increase in glutamate compared with the wild type control.

The nucleic acid sequence of B1445 from Escherichia coli, e.g. as shown in the respective line in column 5 of Table I, application no. 2, has been published in Blattner et al., Science 277 (5331), 1453 (1997)). And the activity of the gene product thereof is the activity of b1445-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “b1445-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1445, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1445, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1445, or a functional equivalent or a homolog thereof as depicted in column 8 of Table II, application no. 2, preferably a homolog or functional equivalent as depicted in column 8 of Table II B, application no. 2, and being depicted in the same respective line as said B1445, and preferably the activity is increased non-targeted.

Accordingly, in one embodiment, the molecule which activity is to be increased in the process of the invention is the gene product with an activity as a “b1445-protein”, preferably it is the molecule of section (a) or (b) of this paragraph. In particular, it was observed that in plants, especially in Arabidopsis thaliana, increasing or generating the activity of a gene product non-targeted with the activity of a “b1445-protein”, preferably being encoded by a gene comprising the nucleic acid sequence SEQ ID NO. 38289, preferably the coding region thereof, conferred the production of or the increase in glutamine compared with the wild type control.

The nucleic acid sequence of B1522 from Escherichia coli, e.g. as shown in the respective line in column 5 of Table I, application no. 2, has been published in Blattner et al., Science 277 (5331), 1453 (1997)). And the activity of the gene product thereof is the activity of b1522-protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “b1522-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1522, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1522, or a functional equivalent or a homolog thereof as depicted in column 8 of Table II, application no. 2, preferably a homolog or functional equivalent as depicted in column 8 of Table II B, application no. 2, and being depicted in the same respective line as said B1522, and preferably the activity is increased non-targeted.

Accordingly, in one embodiment, the molecule which activity is to be increased in the process of the invention is the gene product with an activity as a “b1522-protein”, preferably it is the molecule of section (a) or (b) of this paragraph.

In particular, it was observed that in plants, especially in Arabidopsis thaliana, increasing or generating the activity of a gene product non-targeted with the activity of a “b1522-protein”, preferably being encoded by a gene comprising the nucleic acid sequence SEQ ID NO. 7947, preferably the coding region thereof, conferred the production of or the increase in glutamate compared with the wild type control.

The nucleic acid sequence of B1597 from Escherichia coli, e.g. as shown in the respective line in column 5 of Table I, application no. 2, has been published in Blattner et al., Science 277 (5331), 1453 (1997)). And the activity of the gene product thereof is the activity of acid shock protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “acid shock protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1597, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1597, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1597, or a functional equivalent or a homolog thereof as depicted in column 8 of Table II, application no. 2, preferably a homolog or functional equivalent as depicted in column 8 of Table II B, application no. 2, and being depicted in the same respective line as said B1597, and preferably the activity is increased non-targeted.

Accordingly, in one embodiment, the molecule which activity is to be increased in the process of the invention is the gene product with an activity as a “acid shock protein”, preferably it is the molecule of section (a) or (b) of this paragraph.

In particular, it was observed that in plants, especially in Arabidopsis thaliana, increasing or generating the activity of a gene product non-targeted with the activity of a “acid shock protein”, preferably being encoded by a gene comprising the nucleic acid sequence SEQ ID NO. 38300, preferably the coding region thereof, conferred the production of or the increase in glutamine compared with the wild type control.

The nucleic acid sequence of B1627 from Escherichia coli, e.g. as shown in the respective line in column 5 of Table I, application no. 2, has been published in Blattner et al., Science 277 (5331), 1453 (1997)). And the activity of the gene product thereof is the activity of electron transport complex protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “electron transport complex protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1627, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1627, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1627, or a functional equivalent or a homolog thereof as depicted in column 8 of Table II, application no. 2, preferably a homolog or functional equivalent as depicted in column 8 of Table II B, application no. 2, and being depicted in the same respective line as said B1627, and preferably the activity is increased non-targeted.

Accordingly, in one embodiment, the molecule which activity is to be increased in the process of the invention is the gene product with an activity as a “electron transport complex protein”, preferably it is the molecule of section (a) or (b) of this paragraph.

In particular, it was observed that in plants, especially in Arabidopsis thaliana, increasing or generating the activity of a gene product non-targeted with the activity of a “electron transport complex protein”, preferably being encoded by a gene comprising the nucleic acid sequence SEQ ID NO. 38345, preferably the coding region thereof, conferred the production of or the increase in glutamine compared with the wild type control.

The nucleic acid sequence of B1845 from Escherichia coli, e.g. as shown in the respective line in column 5 of Table I, application no. 2, has been published in Blattner et al., Science 277 (5331), 1453 (1997)). And the activity of the gene product thereof is the activity of protease.

Accordingly, in one embodiment, the process of the present invention for producing glutamate in a non-human organism, like a microorganism or a plant or a part thereof, comprises increasing or generating the activity of a gene product with the activity of a gene product conferring the activity of “protease”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1845, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1845, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1845, or a functional equivalent or a homolog thereof as depicted in column 8 of Table II, application no. 2, preferably a homolog or functional equivalent as depicted in column 8 of Table II B, application no. 2, and being depicted in the same respective line as said B1845, and preferably the activity is increased non-targeted.

Accordingly, in one embodiment, the molecule which activity is to be increased in the process of the invention is the gene product with an activity as a “protease”, preferably it is the molecule of section (a) or (b) of this paragraph.

In particular, it was observed that in plants, especially in Arabidopsis thaliana, increasing or generating the activity of a gene product non-targeted with the activity of a “protease”, preferably being encoded by a gene comprising the nucleic acid sequence SEQ ID NO. 38573, preferably the coding region thereof, conferred the production of or the increase in glutamate compared with the wild type control.

The nucleic acid sequence of B1898 from Escherichia coli, e.g. as shown in the respective line in column 5 of Table I, application no. 2, has been published in Blattner et al., Science 277 (5331), 1453 (1997)). And the activity of the gene product thereof is the activity of b1898-protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “b1898-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1898, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1898, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1898, or a functional equivalent or a homolog thereof as depicted in column 8 of Table II, application no. 2, preferably a homolog or functional equivalent as depicted in column 8 of Table II B, application no. 2, and being depicted in the same respective line as said B1898, and preferably the activity is increased non-targeted.

Accordingly, in one embodiment, the molecule which activity is to be increased in the process of the invention is the gene product with an activity as a “b1898-protein”, preferably it is the molecule of section (a) or (b) of this paragraph.

In particular, it was observed that in plants, especially in Arabidopsis thaliana, increasing or generating the activity of a gene product non-targeted with the activity of a “b1898-protein”, preferably being encoded by a gene comprising the nucleic acid sequence SEQ ID NO. 38767, preferably the coding region thereof, conferred the production of or the increase in glutamate compared with the wild type control.

The nucleic acid sequence of B1981 from Escherichia coli, e.g. as shown in the respective line in column 5 of Table I, application no. 2, has been published in Blattner et al., Science 277 (5331), 1453 (1997)). And the activity of the gene product thereof is the activity of transport protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “transport protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B1981, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B1981, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B1981, or a functional equivalent or a homolog thereof as depicted in column 8 of Table II, application no. 2, preferably a homolog or functional equivalent as depicted in column 8 of Table II B, application no. 2, and being depicted in the same respective line as said B1981, and preferably the activity is increased non-targeted.

Accordingly, in one embodiment, the molecule which activity is to be increased in the process of the invention is the gene product with an activity as a “transport protein”, preferably it is the molecule of section (a) or (b) of this paragraph.

In particular, it was observed that in plants, especially in Arabidopsis thaliana, increasing or generating the activity of a gene product non-targeted with the activity of a “transport protein”, preferably being encoded by a gene comprising the nucleic acid sequence SEQ ID NO. 38899, preferably the coding region thereof, conferred the production of or the increase in glutamine compared with the wild type control.

The nucleic acid sequence of B2063 from Escherichia coli, e.g. as shown in the respective line in column 5 of Table I, application no. 2, has been published in Blattner et al., Science 277 (5331), 1453 (1997)). And the activity of the gene product thereof is the activity of transport protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “transport protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2063, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2063, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2063, or a functional equivalent or a homolog thereof as depicted in column 8 of Table II, application no. 2, preferably a homolog or functional equivalent as depicted in column 8 of Table II B, application no. 2, and being depicted in the same respective line as said B2063, and preferably the activity is increased non-targeted.

Accordingly, in one embodiment, the molecule which activity is to be increased in the process of the invention is the gene product with an activity as a “transport protein”, preferably it is the molecule of section (a) or (b) of this paragraph.

In particular, it was observed that in plants, especially in Arabidopsis thaliana, increasing or generating the activity of a gene product non-targeted with the activity of a “transport protein”, preferably being encoded by a gene comprising the nucleic acid sequence SEQ ID NO. 38947, preferably the coding region thereof, conferred the production of or the increase in glutamate compared with the wild type control.

The nucleic acid sequence of B2063 from Escherichia coli, e.g. as shown in the respective line in column 5 of Table I, application no. 2, has been published in Blattner et al., Science 277 (5331), 1453 (1997)). And the activity of the gene product thereof is the activity of transport protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “transport protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2063, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2063, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2063, or a functional equivalent or a homolog thereof as depicted in column 8 of Table II, application no. 2, preferably a homolog or functional equivalent as depicted in column 8 of Table II B, application no. 2, and being depicted in the same respective line as said B2063, and preferably the activity is increased non-targeted.

Accordingly, in one embodiment, the molecule which activity is to be increased in the process of the invention is the gene product with an activity as a “transport protein”, preferably it is the molecule of section (a) or (b) of this paragraph.

In particular, it was observed that in plants, especially in Arabidopsis thaliana, increasing or generating the activity of a gene product non-targeted with the activity of a “transport protein”, preferably being encoded by a gene comprising the nucleic acid sequence SEQ ID NO. 38947, preferably the coding region thereof, conferred the production of or the increase in glutamine compared with the wild type control.

The nucleic acid sequence of B2063 from Escherichia coli, e.g. as shown in the respective line in column 5 of Table I, application no. 2, has been published in Blattner et al., Science 277 (5331), 1453 (1997)). And the activity of the gene product thereof is the activity of transport protein.

Accordingly, in one embodiment, the process of the present invention for producing proline in a non-human organism, like a microorganism or a plant or a part thereof, comprises increasing or generating the activity of a gene product with the activity of a gene product conferring the activity of “transport protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2063, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2063, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2063, or a functional equivalent or a homolog thereof as depicted in column 8 of Table II, application no. 2, preferably a homolog or functional equivalent as depicted in column 8 of Table II B, application no. 2, and being depicted in the same respective line as said B2063, and preferably the activity is increased non-targeted.

Accordingly, in one embodiment, the molecule which activity is to be increased in the process of the invention is the gene product with an activity as a “transport protein”, preferably it is the molecule of section (a) or (b) of this paragraph.

In particular, it was observed that in plants, especially in Arabidopsis thaliana, increasing or generating the activity of a gene product non-targeted with the activity of a “transport protein”, preferably being encoded by a gene comprising the nucleic acid sequence SEQ ID NO. 38947, preferably the coding region thereof, conferred the production of or the increase in proline compared with the wild type control.

The nucleic acid sequence of B2066 from Escherichia coli, e.g. as shown in the respective line in column 5 of Table I, application no. 2, has been published in S Blattner et al., Science 277 (5331), 1453 (1997)). And the activity of the gene product thereof is the activity of uridine/cytidine kinase.

Accordingly, in one embodiment, the process of the present invention for producing glutamine in a non-human organism, like a microorganism or a plant or a part thereof, comprises increasing or generating the activity of a gene product with the activity of a gene product conferring the activity of “uridine/cytidine kinase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2066, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2066, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2066, or a functional equivalent or a homolog thereof as depicted in column 8 of Table II, application no. 2, preferably a homolog or functional equivalent as depicted in column 8 of Table II B, application no. 2, and being depicted in the same respective line as said B2066, and preferably the activity is increased mitochondrial.

Accordingly, in one embodiment, the molecule which activity is to be increased in the process of the invention is the gene product with an activity as a “uridine/cytidine kinase”, preferably it is the molecule of section (a) or (b) of this paragraph.
In particular, it was observed that in plants, especially in Arabidopsis thaliana, increasing or generating the activity of a gene product mitochondrial with the activity of a “uridine/cytidine kinase”, preferably being encoded by a gene comprising the nucleic acid sequence SEQ ID NO. 8937, preferably the coding region thereof, conferred the production of or the increase in glutamine compared with the wild type control.

The nucleic acid sequence of B2107 from Escherichia coli, e.g. as shown in the respective line in column 5 of Table I, application no. 2, has been published in Blattner et al., Science 277 (5331), 1453 (1997)). And the activity of the gene product thereof is the activity of b2107-protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “b2107-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2107, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2107, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2107, or a functional equivalent or a homolog thereof as depicted in column 8 of Table II, application no. 2, preferably a homolog or functional equivalent as depicted in column 8 of Table II B, application no. 2, and being depicted in the same respective line as said B2107, and preferably the activity is increased non-targeted.

Accordingly, in one embodiment, the molecule which activity is to be increased in the process of the invention is the gene product with an activity as a “b2107-protein”, preferably it is the molecule of section (a) or (b) of this paragraph.

In particular, it was observed that in plants, especially in Arabidopsis thaliana, increasing or generating the activity of a gene product non-targeted with the activity of a “b2107-protein”, preferably being encoded by a gene comprising the nucleic acid sequence SEQ ID NO. 39002, preferably the coding region thereof, conferred the production of or the increase in glutamate compared with the wild type control.

The nucleic acid sequence of B2121 from Escherichia coli, e.g. as shown in the respective line in column 5 of Table I, application no. 2, has been published in Blattner et al., Science 277 (5331), 1453 (1997)). And the activity of the gene product thereof is the activity of b2121-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “b2121-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2121, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2121, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2121, or a functional equivalent or a homolog thereof as depicted in column 8 of Table II, application no. 2, preferably a homolog or functional equivalent as depicted in column 8 of Table II B, application no. 2, and being depicted in the same respective line as said B2121, and preferably the activity is increased non-targeted.

Accordingly, in one embodiment, the molecule which activity is to be increased in the process of the invention is the gene product with an activity as a “b2121-protein”, preferably it is the molecule of section (a) or (b) of this paragraph.

In particular, it was observed that in plants, especially in Arabidopsis thaliana, increasing or generating the activity of a gene product non-targeted with the activity of a “b2121-protein”, preferably being encoded by a gene comprising the nucleic acid sequence SEQ ID NO. 39013, preferably the coding region thereof, conferred the production of or the increase in glutamine compared with the wild type control.

The nucleic acid sequence of B2178 from Escherichia coli, e.g. as shown in the respective line in column 5 of Table I, application no. 2, has been published in Blattner et al., Science 277 (5331), 1453 (1997)). And the activity of the gene product thereof is the activity of ABC transporter permease protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “ABC transporter permease protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2178, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2178, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2178, or a functional equivalent or a homolog thereof as depicted in column 8 of Table II, application no. 2, preferably a homolog or functional equivalent as depicted in column 8 of Table II B, application no. 2, and being depicted in the same respective line as said B2178, and preferably the activity is increased non-targeted.

Accordingly, in one embodiment, the molecule which activity is to be increased in the process of the invention is the gene product with an activity as a “ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “ABC transporter permease protein”, preferably being encoded by a gene comprising the nucleic acid sequence SEQ ID NO. 39040, preferably the coding region thereof, conferred the production of or the increase in glutamate compared with the wild type control.

The nucleic acid sequence of B2178 from Escherichia coli, e.g. as shown in the respective line in column 5 of Table I, application no. 2, has been published in Blattner et al., Science 277 (5331), 1453 (1997)). And the activity of the gene product thereof is the activity of ABC transporter permease protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “ABC transporter permease protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2178, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2178, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2178, or a functional equivalent or a homolog thereof as depicted in column 8 of Table II, application no. 2, preferably a homolog or functional equivalent as depicted in column 8 of Table II B, application no. 2, and being depicted in the same respective line as said B2178, and preferably the activity is increased non-targeted.

Accordingly, in one embodiment, the molecule which activity is to be increased in the process of the invention is the gene product with an activity as a “ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “ABC transporter permease protein”, preferably being encoded by a gene comprising the nucleic acid sequence SEQ ID NO. 39040, preferably the coding region thereof, conferred the production of or the increase in glutamine compared with the wild type control.

The nucleic acid sequence of B2178 from Escherichia coli, e.g. as shown in the respective line in column 5 of Table I, application no. 2, has been published in Blattner et al., Science 277 (5331), 1453 (1997)). And the activity of the gene product thereof is the activity of ABC transporter permease protein.

Accordingly, in one embodiment, the process of the present invention for producing proline in a non-human organism, like a microorganism or a plant or a part thereof, comprises increasing or generating the activity of a gene product with the activity of a gene product conferring the activity of “ABC transporter permease protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2178, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2178, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2178, or a functional equivalent or a homolog thereof as depicted in column 8 of Table II, application no. 2, preferably a homolog or functional equivalent as depicted in column 8 of Table II B, application no. 2, and being depicted in the same respective line as said B2178, and preferably the activity is increased non-targeted.

Accordingly, in one embodiment, the molecule which activity is to be increased in the process of the invention is the gene product with an activity as a “ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “ABC transporter permease protein”, preferably being encoded by a gene comprising the nucleic acid sequence SEQ ID NO. 39040, preferably the coding region thereof, conferred the production of or the increase in proline compared with the wild type control.

The nucleic acid sequence of B2281 from Escherichia coli, e.g. as shown in the respective line in column 5 of Table I, application no. 2, has been published in Blattner et al., Science 277 (5331), 1453 (1997)). And the activity of the gene product thereof is the activity of NADH dehydrogenase I chain I.

Accordingly, in one embodiment, the process of the present invention for producing glutamine in a non-human organism, like a microorganism or a plant or a part thereof, comprises increasing or generating the activity of a gene product with the activity of a gene product conferring the activity of “NADH dehydrogenase I chain I”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2281, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2281, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2281, or a functional equivalent or a homolog thereof as depicted in column 8 of Table II, application no. 2, preferably a homolog or functional equivalent as depicted in column 8 of Table II B, application no. 2, and being depicted in the same respective line as said B2281, and preferably the activity is increased non-targeted.

Accordingly, in one embodiment, the molecule which activity is to be increased in the process of the invention is the gene product with an activity as a “NADH dehydrogenase I chain I”, preferably it is the molecule of section (a) or (b) of this paragraph.

In particular, it was observed that in plants, especially in Arabidopsis thaliana, increasing or generating the activity of a gene product non-targeted with the activity of a “NADH dehydrogenase I chain I”, preferably being encoded by a gene comprising the nucleic acid sequence SEQ ID NO. 39120, preferably the coding region thereof, conferred the production of or the increase in glutamine compared with the wild type control.

The nucleic acid sequence of B2360 from Escherichia coli, e.g. as shown in the respective line in column 5 of Table I, application no. 2, has been published in Blattner et al., Science 277 (5331), 1453 (1997)). And the activity of the gene product thereof is the activity of b2360-protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “b2360-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2360, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2360, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2360, or a functional equivalent or a homolog thereof as depicted in column 8 of Table II, application no. 2, preferably a homolog or functional equivalent as depicted in column 8 of Table II B, application no. 2, and being depicted in the same respective line as said B2360, and preferably the activity is increased non-targeted.

Accordingly, in one embodiment, the molecule which activity is to be increased in the process of the invention is the gene product with an activity as a “b2360-protein”, preferably it is the molecule of section (a) or (b) of this paragraph.

In particular, it was observed that in plants, especially in Arabidopsis thaliana, increasing or generating the activity of a gene product non-targeted with the activity of a “b2360-protein”, preferably being encoded by a gene comprising the nucleic acid sequence SEQ ID NO. 39219, preferably the coding region thereof, conferred the production of or the increase in glutamate compared with the wild type control.

The nucleic acid sequence of B2399 from Escherichia coli, e.g. as shown in the respective line in column 5 of Table I, application no. 2, has been published in Blattner et al., Science 277 (5331), 1453 (1997)). And the activity of the gene product thereof is the activity of b2399-protein.

Accordingly, in one embodiment, the process of the present invention for producing proline in a non-human organism, like a microorganism or a plant or a part thereof, comprises increasing or generating the activity of a gene product with the activity of a gene product conferring the activity of “b2399-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2399, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2399, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2399, or a functional equivalent or a homolog thereof as depicted in column 8 of Table II, application no. 2, preferably a homolog or functional equivalent as depicted in column 8 of Table II B, application no. 2, and being depicted in the same respective line as said B2399, and preferably the activity is increased non-targeted.

Accordingly, in one embodiment, the molecule which activity is to be increased in the process of the invention is the gene product with an activity as a “b2399-protein”, preferably it is the molecule of section (a) or (b) of this paragraph.

In particular, it was observed that in plants, especially in Arabidopsis thaliana, increasing or generating the activity of a gene product non-targeted with the activity of a “b2399-protein”, preferably being encoded by a gene comprising the nucleic acid sequence SEQ ID NO. 39237, preferably the coding region thereof, conferred the production of or the increase in proline compared with the wild type control.

The nucleic acid sequence of B2405 from Escherichia coli, e.g. as shown in the respective line in column 5 of Table I, application no. 2, has been published in Blattner et al., Science 277 (5331), 1453 (1997)). And the activity of the gene product thereof is the activity of transcriptional regulator.

Accordingly, in one embodiment, the process of the present invention for producing glutamate in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2405, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2405, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2405, or a functional equivalent or a homolog thereof as depicted in column 8 of Table II, application no. 2, preferably a homolog or functional equivalent as depicted in column 8 of Table II B, application no. 2, and being depicted in the same respective line as said B2405, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 39255, preferably the coding region thereof, conferred the production of or the increase in glutamate compared with the wild type control.

The nucleic acid sequence of B2414 from Escherichia coli, e.g. as shown in the respective line in column 5 of Table I, application no. 2, has been published in Blattner et al., Science 277 (5331), 1453 (1997)). And the activity of the gene product thereof is the activity of cysteine synthase A.

Accordingly, in one embodiment, the process of the present invention for producing arginine in a non-human organism, like a microorganism or a plant or a part thereof, comprises increasing or generating the activity of a gene product with the activity of a gene product conferring the activity of “cysteine synthase A”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2414, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2414, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2414, or a functional equivalent or a homolog thereof as depicted in column 8 of Table II, application no. 2, preferably a homolog or functional equivalent as depicted in column 8 of Table II B, application no. 2, and being depicted in the same respective line as said B2414, and preferably the activity is increased non-targeted.

Accordingly, in one embodiment, the molecule which activity is to be increased in the process of the invention is the gene product with an activity as a “cysteine synthase A”, preferably it is the molecule of section (a) or (b) of this paragraph.

In particular, it was observed that in plants, especially in Arabidopsis thaliana, increasing or generating the activity of a gene product non-targeted with the activity of a “cysteine synthase A”, preferably being encoded by a gene comprising the nucleic acid sequence SEQ ID NO. 39300, preferably the coding region thereof, conferred the production of or the increase in arginine compared with the wild type control.

The nucleic acid sequence of B2414 from Escherichia coli, e.g. as shown in the respective line in column 5 of Table I, application no. 2, has been published in Blattner et al., Science 277 (5331), 1453 (1997)). And the activity of the gene product thereof is the activity of cysteine synthase A.

Accordingly, in one embodiment, the process of the present invention for producing glutamate in a non-human organism, like a microorganism or a plant or a part thereof, comprises increasing or generating the activity of a gene product with the activity of a gene product conferring the activity of “cysteine synthase A”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2414, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2414, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2414, or a functional equivalent or a homolog thereof as depicted in column 8 of Table II, application no. 2, preferably a homolog or functional equivalent as depicted in column 8 of Table II B, application no. 2, and being depicted in the same respective line as said B2414, and preferably the activity is increased non-targeted.

Accordingly, in one embodiment, the molecule which activity is to be increased in the process of the invention is the gene product with an activity as a “cysteine synthase A”, preferably it is the molecule of section (a) or (b) of this paragraph.

In particular, it was observed that in plants, especially in Arabidopsis thaliana, increasing or generating the activity of a gene product non-targeted with the activity of a “cysteine synthase A”, preferably being encoded by a gene comprising the nucleic acid sequence SEQ ID NO. 39300, preferably the coding region thereof, conferred the production of or the increase in glutamate compared with the wild type control.

The nucleic acid sequence of B2461 from Escherichia coli, e.g. as shown in the respective line in column 5 of Table I, application no. 2, has been published in Blattner et al., Science 277 (5331), 1453 (1997)). And the activity of the gene product thereof is the activity of ethanolamine utilization protein.

Accordingly, in one embodiment, the process of the present invention for producing proline in a non-human organism, like a microorganism or a plant or a part thereof, comprises increasing or generating the activity of a gene product with the activity of a gene product conferring the activity of “ethanolamine utilization protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2461, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2461, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2461, or a functional equivalent or a homolog thereof as depicted in column 8 of Table II, application no. 2, preferably a homolog or functional equivalent as depicted in column 8 of Table II B, application no. 2, and being depicted in the same respective line as said B2461, and preferably the activity is increased non-targeted.

Accordingly, in one embodiment, the molecule which activity is to be increased in the process of the invention is the gene product with an activity as a “ethanolamine utilization protein”, preferably it is the molecule of section (a) or (b) of this paragraph.

In particular, it was observed that in plants, especially in Arabidopsis thaliana, increasing or generating the activity of a gene product non-targeted with the activity of a “ethanolamine utilization protein”, preferably being encoded by a gene comprising the nucleic acid sequence SEQ ID NO. 40299, preferably the coding region thereof, conferred the production of or the increase in proline compared with the wild type control.

The nucleic acid sequence of B2474 from Escherichia coli, e.g. as shown in the respective line in column 5 of Table I, application no. 2, has been published in Blattner et al., Science 277 (5331), 1453 (1997)). And the activity of the gene product thereof is the activity of b2474-protein.

Accordingly, in one embodiment, the process of the present invention for producing proline in a non-human organism, like a microorganism or a plant or a part thereof, comprises increasing or generating the activity of a gene product with the activity of a gene product conferring the activity of “b2474-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2474, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2474, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2474, or a functional equivalent or a homolog thereof as depicted in column 8 of Table II, application no. 2, preferably a homolog or functional equivalent as depicted in column 8 of Table II B, application no. 2, and being depicted in the same respective line as said B2474, and preferably the activity is increased non-targeted,.

Accordingly, in one embodiment, the molecule which activity is to be increased in the process of the invention is the gene product with an activity as a “b2474-protein”, preferably it is the molecule of section (a) or (b) of this paragraph.

In particular, it was observed that in plants, especially in Arabidopsis thaliana, increasing or generating the activity of a gene product non-targeted with the activity of a “b2474-protein”, preferably being encoded by a gene comprising the nucleic acid sequence SEQ ID NO. 40329, preferably the coding region thereof, conferred the production of or the increase in proline compared with the wild type control.

The nucleic acid sequence of B2513 from Escherichia coli, e.g. as shown in the respective line in column 5 of Table I, application no. 2, has been published in Blattner et al., Science 277 (5331), 1453 (1997)). And the activity of the gene product thereof is the activity of b2513-protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “b2513-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2513, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2513, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2513, or a functional equivalent or a homolog thereof as depicted in column 8 of Table II, application no. 2, preferably a homolog or functional equivalent as depicted in column 8 of Table II B, application no. 2, and being depicted in the same respective line as said B2513, and preferably the activity is increased non-targeted.

Accordingly, in one embodiment, the molecule which activity is to be increased in the process of the invention is the gene product with an activity as a “b2513-protein”, preferably it is the molecule of section (a) or (b) of this paragraph.

In particular, it was observed that in plants, especially in Arabidopsis thaliana, increasing or generating the activity of a gene product non-targeted with the activity of a “b2513-protein”, preferably being encoded by a gene comprising the nucleic acid sequence SEQ ID NO. 9167, preferably the coding region thereof, conferred the production of or the increase in glutamate compared with the wild type control.

The nucleic acid sequence of B2513 from Escherichia coli, e.g. as shown in the respective line in column 5 of Table I, application no. 2, has been published in Blattner et al., Science 277 (5331), 1453 (1997)). And the activity of the gene product thereof is the activity of b2513-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “b2513-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2513, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2513, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2513, or a functional equivalent or a homolog thereof as depicted in column 8 of Table II, application no. 2, preferably a homolog or functional equivalent as depicted in column 8 of Table II B, application no. 2, and being depicted in the same respective line as said B2513, and preferably the activity is increased non-targeted.

Accordingly, in one embodiment, the molecule which activity is to be increased in the process of the invention is the gene product with an activity as a “b2513-protein”, preferably it is the molecule of section (a) or (b) of this paragraph.

In particular, it was observed that in plants, especially in Arabidopsis thaliana, increasing or generating the activity of a gene product non-targeted with the activity of a “b2513-protein”, preferably being encoded by a gene comprising the nucleic acid sequence SEQ ID NO. 9167, preferably the coding region thereof, conferred the production of or the increase in glutamine compared with the wild type control.

The nucleic acid sequence of B2541 from Escherichia coli, e.g. as shown in the respective line in column 5 of Table I, application no. 2, has been published in Blattner et al., Science 277 (5331), 1453 (1997)). And the activity of the gene product thereof is the activity of 2,3-dihydroxy-2,3-dihydrophenylpropionatedehydrogenase.

Accordingly, in one embodiment, the process of the present invention for producing arginine in a non-human organism, like a microorganism or a plant or a part thereof, comprises increasing or generating the activity of a gene product with the activity of a gene product conferring the activity of “2,3-dihydroxy-2,3-dihydrophenylpropionatedehydrogenase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2541, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2541, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2541, or a functional equivalent or a homolog thereof as depicted in column 8 of Table II, application no. 2, preferably a homolog or functional equivalent as depicted in column 8 of Table II B, application no. 2, and being depicted in the same respective line as said B2541, and preferably the activity is increased non-targeted.

Accordingly, in one embodiment, the molecule which activity is to be increased in the process of the invention is the gene product with an activity as a “2,3-dihydroxy-2,3-dihydrophenylpropionatedehydrogenase”, preferably it is the molecule of section (a) or (b) of this paragraph.

In particular, it was observed that in plants, especially in Arabidopsis thaliana, increasing or generating the activity of a gene product non-targeted with the activity of a “2,3-dihydroxy-2,3-dihydrophenylpropionatedehydrogenase”, preferably being encoded by a gene comprising the nucleic acid sequence SEQ ID NO. 40383, preferably the coding region thereof, conferred the production of or the increase in arginine compared with the wild type control.

CHECK The nucleic acid sequence of B2541 from Escherichia coli, e.g. as shown in the respective line in column 5 of Table I, application no. 2, has been published in Blattner et al., Science 277 (5331), 1453 (1997)). And the activity of the gene product thereof is the activity of 2,3-dihydroxy-2,3-dihydrophenylpropionatedehydrogenase.

Accordingly, in one embodiment, the process of the present invention for producing glutamate in a non-human organism, like a microorganism or a plant or a part thereof, comprises increasing or generating the activity of a gene product with the activity of a gene product conferring the activity of “2,3-dihydroxy-2,3-dihydrophenylpropionatedehydrogenase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2541, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2541, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2541, or a functional equivalent or a homolog thereof as depicted in column 8 of Table II, application no. 2, preferably a homolog or functional equivalent as depicted in column 8 of Table II B, application no. 2, and being depicted in the same respective line as said B2541, and preferably the activity is increased non-targeted.

Accordingly, in one embodiment, the molecule which activity is to be increased in the process of the invention is the gene product with an activity as a “2,3-dihydroxy-2,3-dihydrophenylpropionatedehydrogenase”, preferably it is the molecule of section (a) or (b) of this paragraph.

In particular, it was observed that in plants, especially in Arabidopsis thaliana, increasing or generating the activity of a gene product non-targeted with the activity of a “2,3-dihydroxy-2,3-dihydrophenylpropionatedehydrogenase”, preferably being encoded by a gene comprising the nucleic acid sequence SEQ ID NO. 40383, preferably the coding region thereof, conferred the production of or the increase in glutamate compared with the wild type control.

The nucleic acid sequence of B2548 from Escherichia coli, e.g. as shown in the respective line in column 5 of Table I, application no. 2, has been published in Blattner et al., Science 277 (5331), 1453 (1997)). And the activity of the gene product thereof is the activity of b2548-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “b2548-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2548, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2548, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2548, or a functional equivalent or a homolog thereof as depicted in column 8 of Table II, application no. 2, preferably a homolog or functional equivalent as depicted in column 8 of Table II B, application no. 2, and being depicted in the same respective line as said B2548, and preferably the activity is increased non-targeted.

Accordingly, in one embodiment, the molecule which activity is to be increased in the process of the invention is the gene product with an activity as a “b2548-protein”, preferably it is the molecule of section (a) or (b) of this paragraph.

In particular, it was observed that in plants, especially in Arabidopsis thaliana, increasing or generating the activity of a gene product non-targeted with the activity of a “b2548-protein”, preferably being encoded by a gene comprising the nucleic acid sequence SEQ ID NO. 40637, preferably the coding region thereof, conferred the production of or the increase in glutamine compared with the wild type control.

The nucleic acid sequence of B2613 from Escherichia coli, e.g. as shown in the respective line in column 5 of Table I, application no. 2, has been published in Blattner et al., Science 277 (5331), 1453 (1997)). And the activity of the gene product thereof is the activity of b2613-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “b2613-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2613, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2613, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2613, or a functional equivalent or a homolog thereof as depicted in column 8 of Table II, application no. 2, preferably a homolog or functional equivalent as depicted in column 8 of Table II B, application no. 2, and being depicted in the same respective line as said B2613, and preferably the activity is increased non-targeted.

Accordingly, in one embodiment, the molecule which activity is to be increased in the process of the invention is the gene product with an activity as a “b2613-protein”, preferably it is the molecule of section (a) or (b) of this paragraph.

In particular, it was observed that in plants, especially in Arabidopsis thaliana, increasing or generating the activity of a gene product non-targeted with the activity of a “b2613-protein”, preferably being encoded by a gene comprising the nucleic acid sequence SEQ ID NO. 40665, preferably the coding region thereof, conferred the production of or the increase in glutamine compared with the wild type control.

The nucleic acid sequence of B2613 from Escherichia coli, e.g. as shown in the respective line in column 5 of Table I, application no. 2, has been published in Blattner et al., Science 277 (5331), 1453 (1997)). And the activity of the gene product thereof is the activity of b2613-protein.

Accordingly, in one embodiment, the process of the present invention for producing proline in a non-human organism, like a microorganism or a plant or a part thereof, comprises increasing or generating the activity of a gene product with the activity of a gene product conferring the activity of “b2613-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2613, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2613, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2613, or a functional equivalent or a homolog thereof as depicted in column 8 of Table II, application no. 2, preferably a homolog or functional equivalent as depicted in column 8 of Table II B, application no. 2, and being depicted in the same respective line as said B2613, and preferably the activity is increased non-targeted.

Accordingly, in one embodiment, the molecule which activity is to be increased in the process of the invention is the gene product with an activity as a “b2613-protein”, preferably it is the molecule of section (a) or (b) of this paragraph.

In particular, it was observed that in plants, especially in Arabidopsis thaliana, increasing or generating the activity of a gene product non-targeted with the activity of a “b2613-protein”, preferably being encoded by a gene comprising the nucleic acid sequence SEQ ID NO. 40665, preferably the coding region thereof, conferred the production of or the increase in proline compared with the wild type control.

The nucleic acid sequence of B2634 from Escherichia coli, e.g. as shown in the respective line in column 5 of Table I, application no. 2, has been published in Blattner et al., Science 277 (5331), 1453 (1997)). And the activity of the gene product thereof is the activity of transcriptional regulator.

Accordingly, in one embodiment, the process of the present invention for producing glutamate in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2634, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2634, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2634, or a functional equivalent or a homolog thereof as depicted in column 8 of Table II, application no. 2, preferably a homolog or functional equivalent as depicted in column 8 of Table II B, application no. 2, and being depicted in the same respective line as said B2634, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 40726, preferably the coding region thereof, conferred the production of or the increase in glutamate compared with the wild type control.

The nucleic acid sequence of B2634 from Escherichia coli, e.g. as shown in the respective line in column 5 of Table I, application no. 2, has been published in Blattner et al., Science 277 (5331), 1453 (1997)). And the activity of the gene product thereof is the activity of transcriptional regulator.

Accordingly, in one embodiment, the process of the present invention for producing glutamine in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2634, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2634, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2634, or a functional equivalent or a homolog thereof as depicted in column 8 of Table II, application no. 2, preferably a homolog or functional equivalent as depicted in column 8 of Table II B, application no. 2, and being depicted in the same respective line as said B2634, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 40726, preferably the coding region thereof, conferred the production of or the increase in glutamine compared with the wild type control.

The nucleic acid sequence of B2634 from Escherichia coli, e.g. as shown in the respective line in column 5 of Table I, application no. 2, has been published in S Blattner et al., Science 277 (5331), 1453 (1997)). And the activity of the gene product thereof is the activity of transcriptional regulator.

Accordingly, in one embodiment, the process of the present invention for producing proline in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2634, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2634, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2634, or a functional equivalent or a homolog thereof as depicted in column 8 of Table II, application no. 2, preferably a homolog or functional equivalent as depicted in column 8 of Table II B, application no. 2, and being depicted in the same respective line as said B2634, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 40726, preferably the coding region thereof, conferred the production of or the increase in proline compared with the wild type control.

The nucleic acid sequence of B2673 from Escherichia coli, e.g. as shown in the respective line in column 5 of Table I, application no. 2, has been published in Blattner et al., Science 277 (5331), 1453 (1997)). And the activity of the gene product thereof is the activity of b2673-protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “b2673-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2673, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2673, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2673, or a functional equivalent or a homolog thereof as depicted in column 8 of Table II, application no. 2, preferably a homolog or functional equivalent as depicted in column 8 of Table II B, application no. 2, and being depicted in the same respective line as said B2673, and preferably the activity is increased non-targeted.

Accordingly, in one embodiment, the molecule which activity is to be increased in the process of the invention is the gene product with an activity as a “b2673-protein”, preferably it is the molecule of section (a) or (b) of this paragraph.

In particular, it was observed that in plants, especially in Arabidopsis thaliana, increasing or generating the activity of a gene product non-targeted with the activity of a “b2673-protein”, preferably being encoded by a gene comprising the nucleic acid sequence SEQ ID NO. 9244, preferably the coding region thereof, conferred the production of or the increase in glutamate compared with the wild type control.

The nucleic acid sequence of B2673 from Escherichia coli, e.g. as shown in the respective line in column 5 of Table I, application no. 2, has been published in Blattner et al., Science 277 (5331), 1453 (1997)). And the activity of the gene product thereof is the activity of b2673-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “b2673-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2673, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2673, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2673, or a functional equivalent or a homolog thereof as depicted in column 8 of Table II, application no. 2, preferably a homolog or functional equivalent as depicted in column 8 of Table II B, application no. 2, and being depicted in the same respective line as said B2673, and preferably the activity is increased non-targeted.

Accordingly, in one embodiment, the molecule which activity is to be increased in the process of the invention is the gene product with an activity as a “b2673-protein”, preferably it is the molecule of section (a) or (b) of this paragraph.

In particular, it was observed that in plants, especially in Arabidopsis thaliana, increasing or generating the activity of a gene product non-targeted with the activity of a “b2673-protein”, preferably being encoded by a gene comprising the nucleic acid sequence SEQ ID NO. 9244, preferably the coding region thereof, conferred the production of or the increase in glutamine compared with the wild type control.

The nucleic acid sequence of B2714 from Escherichia coli, e.g. as shown in the respective line in column 5 of Table I, application no. 2, has been published in Blattner et al., Science 277 (5331), 1453 (1997)). And the activity of the gene product thereof is the activity of transcriptional regulator.

Accordingly, in one embodiment, the process of the present invention for producing glutamine in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2714, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2714, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2714, or a functional equivalent or a homolog thereof as depicted in column 8 of Table II, application no. 2, preferably a homolog or functional equivalent as depicted in column 8 of Table II B, application no. 2, and being depicted in the same respective line as said B2714, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 40741, preferably the coding region thereof, conferred the production of or the increase in glutamine compared with the wild type control.

The nucleic acid sequence of B2714 from Escherichia coli, e.g. as shown in the respective line in column 5 of Table I, application no. 2, has been published in Blattner et al., Science 277 (5331), 1453 (1997)). And the activity of the gene product thereof is the activity of transcriptional regulator.

Accordingly, in one embodiment, the process of the present invention for producing proline in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2714, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2714, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2714, or a functional equivalent or a homolog thereof as depicted in column 8 of Table II, application no. 2, preferably a homolog or functional equivalent as depicted in column 8 of Table II B, application no. 2, and being depicted in the same respective line as said B2714, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 40741, preferably the coding region thereof, conferred the production of or the increase in proline compared with the wild type control.

The nucleic acid sequence of B2812 from Escherichia coli, e.g. as shown in the respective line in column 5 of Table I, application no. 2, has been published in Blattner et al., Science 277 (5331), 1453 (1997)). And the activity of the gene product thereof is the activity of b2812-protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “b2812-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2812, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2812, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2812, or a functional equivalent or a homolog thereof as depicted in column 8 of Table II, application no. 2, preferably a homolog or functional equivalent as depicted in column 8 of Table II B, application no. 2, and being depicted in the same respective line as said B2812, and preferably the activity is increased non-targeted.

Accordingly, in one embodiment, the molecule which activity is to be increased in the process of the invention is the gene product with an activity as a “b2812-protein”, preferably it is the molecule of section (a) or (b) of this paragraph.

In particular, it was observed that in plants, especially in Arabidopsis thaliana, increasing or generating the activity of a gene product non-targeted with the activity of a “b2812-protein”, preferably being encoded by a gene comprising the nucleic acid sequence SEQ ID NO. 40795, preferably the coding region thereof, conferred the production of or the increase in glutamate compared with the wild type control.

The nucleic acid sequence of B2812 from Escherichia coli, e.g. as shown in the respective line in column 5 of Table I, application no. 2, has been published in Blattner et al., Science 277 (5331), 1453 (1997)). And the activity of the gene product thereof is the activity of b2812-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “b2812-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2812, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2812, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2812, or a functional equivalent or a homolog thereof as depicted in column 8 of Table II, application no. 2, preferably a homolog or functional equivalent as depicted in column 8 of Table II B, application no. 2, and being depicted in the same respective line as said B2812, and preferably the activity is increased non-targeted.

Accordingly, in one embodiment, the molecule which activity is to be increased in the process of the invention is the gene product with an activity as a “b2812-protein”, preferably it is the molecule of section (a) or (b) of this paragraph.

In particular, it was observed that in plants, especially in Arabidopsis thaliana, increasing or generating the activity of a gene product non-targeted with the activity of a “b2812-protein”, preferably being encoded by a gene comprising the nucleic acid sequence SEQ ID NO. 40795, preferably the coding region thereof, conferred the production of or the increase in glutamine compared with the wild type control.

The nucleic acid sequence of B2812 from Escherichia coli, e.g. as shown in the respective line in column 5 of Table I, application no. 2, has been published in Blattner et al., Science 277 (5331), 1453 (1997)) . And the activity of the gene product thereof is the activity of b2812-protein.

Accordingly, in one embodiment, the process of the present invention for producing proline in a non-human organism, like a microorganism or a plant or a part thereof, comprises increasing or generating the activity of a gene product with the activity of a gene product conferring the activity of “b2812-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2812, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2812, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2812, or a functional equivalent or a homolog thereof as depicted in column 8 of Table II, application no. 2, preferably a homolog or functional equivalent as depicted in column 8 of Table II B, application no. 2, and being depicted in the same respective line as said B2812, and preferably the activity is increased non-targeted.

Accordingly, in one embodiment, the molecule which activity is to be increased in the process of the invention is the gene product with an activity as a “b2812-protein”, preferably it is the molecule of section (a) or (b) of this paragraph.

In particular, it was observed that in plants, especially in Arabidopsis thaliana, increasing or generating the activity of a gene product non-targeted with the activity of a “b2812-protein”, preferably being encoded by a gene comprising the nucleic acid sequence SEQ ID NO. 40795, preferably the coding region thereof, conferred the production of or the increase in proline compared with the wild type control.

The nucleic acid sequence of B2846 from Escherichia coli, e.g. as shown in the respective line in column 5 of Table I, application no. 2, has been published in Blattner et al., Science 277 (5331), 1453 (1997)). And the activity of the gene product thereof is the activity of b2846-protein.

Accordingly, in one embodiment, the process of the present invention for producing proline in a non-human organism, like a microorganism or a plant or a part thereof, comprises increasing or generating the activity of a gene product with the activity of a gene product conferring the activity of “b2846-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2846, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2846, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2846, or a functional equivalent or a homolog thereof as depicted in column 8 of Table II, application no. 2, preferably a homolog or functional equivalent as depicted in column 8 of Table II B, application no. 2, and being depicted in the same respective line as said B2846, and preferably the activity is increased non-targeted.

Accordingly, in one embodiment, the molecule which activity is to be increased in the process of the invention is the gene product with an activity as a “b2846-protein”, preferably it is the molecule of section (a) or (b) of this paragraph.

In particular, it was observed that in plants, especially in Arabidopsis thaliana, increasing or generating the activity of a gene product non-targeted with the activity of a “b2846-protein”, preferably being encoded by a gene comprising the nucleic acid sequence SEQ ID NO. 40984, preferably the coding region thereof, conferred the production of or the increase in proline compared with the wild type control.

CHECK The nucleic acid sequence of B2909 from Escherichia coli, e.g. as shown in the respective line in column 5 of Table I, application no. 2, has been published in Blattner et al., Science 277 (5331), 1453 (1997)). And the activity of the gene product thereof is the activity of b2909-protein.

Accordingly, in one embodiment, the process of the present invention for producing arginine in a non-human organism, like a microorganism or a plant or a part thereof, comprises increasing or generating the activity of a gene product with the activity of a gene product conferring the activity of “b2909-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2909, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2909, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2909, or a functional equivalent or a homolog thereof as depicted in column 8 of Table II, application no. 2, preferably a homolog or functional equivalent as depicted in column 8 of Table II B, application no. 2, and being depicted in the same respective line as said B2909, and preferably the activity is increased non-targeted.

Accordingly, in one embodiment, the molecule which activity is to be increased in the process of the invention is the gene product with an activity as a “b2909-protein”, preferably it is the molecule of section (a) or (b) of this paragraph.

In particular, it was observed that in plants, especially in Arabidopsis thaliana, increasing or generating the activity of a gene product non-targeted with the activity of a “b2909-protein”, preferably being encoded by a gene comprising the nucleic acid sequence SEQ ID NO. 41006, preferably the coding region thereof, conferred the production of or the increase in arginine compared with the wild type control.

The nucleic acid sequence of B2909 from Escherichia coli, e.g. as shown in the respective line in column 5 of Table I, application no. 2, has been published in Blattner et al., Science 277 (5331), 1453 (1997)). And the activity of the gene product thereof is the activity of b2909-protein.

Accordingly, in one embodiment, the process of the present invention for producing proline in a non-human organism, like a microorganism or a plant or a part thereof, comprises increasing or generating the activity of a gene product with the activity of a gene product conferring the activity of “b2909-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2909, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2909, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2909, or a functional equivalent or a homolog thereof as depicted in column 8 of Table II, application no. 2, preferably a homolog or functional equivalent as depicted in column 8 of Table II B, application no. 2, and being depicted in the same respective line as said B2909, and preferably the activity is increased non-targeted.

Accordingly, in one embodiment, the molecule which activity is to be increased in the process of the invention is the gene product with an activity as a “b2909-protein”, preferably it is the molecule of section (a) or (b) of this paragraph.

In particular, it was observed that in plants, especially in Arabidopsis thaliana, increasing or generating the activity of a gene product non-targeted with the activity of a “b2909-protein”, preferably being encoded by a gene comprising the nucleic acid sequence SEQ ID NO. 41006, preferably the coding region thereof, conferred the production of or the increase in proline compared with the wild type control.

The nucleic acid sequence of B2914 from Escherichia coli, e.g. as shown in the respective line in column 5 of Table I, application no. 2, has been published in Blattner et al., Science 277 (5331), 1453 (1997)). And the activity of the gene product thereof is the activity of ribosephosphate isomerase, constitutive.

Accordingly, in one embodiment, the process of the present invention for producing glutamate in a non-human organism, like a microorganism or a plant or a part thereof, comprises increasing or generating the activity of a gene product with the activity of a gene product conferring the activity of “ribosephosphate isomerase, constitutive”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2914, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2914, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2914, or a functional equivalent or a homolog thereof as depicted in column 8 of Table II, application no. 2, preferably a homolog or functional equivalent as depicted in column 8 of Table II B, application no. 2, and being depicted in the same respective line as said B2914, and preferably the activity is increased non-targeted.

Accordingly, in one embodiment, the molecule which activity is to be increased in the process of the invention is the gene product with an activity as a “ribosephosphate isomerase, constitutive”, preferably it is the molecule of section (a) or (b) of this paragraph.

In particular, it was observed that in plants, especially in Arabidopsis thaliana, increasing or generating the activity of a gene product non-targeted with the activity of a “ribosephosphate isomerase, constitutive”, preferably being encoded by a gene comprising the nucleic acid sequence SEQ ID NO. 41073, preferably the coding region thereof, conferred the production of or the increase in glutamate compared with the wild type control.

The nucleic acid sequence of B2923 from Escherichia coli, e.g. as shown in the respective line in column 5 of Table I, application no. 2, has been published in Blattner et al., Science 277 (5331), 1453 (1997)). And the activity of the gene product thereof is the activity of arginine exporter protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “arginine exporter protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2923, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2923, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2923, or a functional equivalent or a homolog thereof as depicted in column 8 of Table II, application no. 2, preferably a homolog or functional equivalent as depicted in column 8 of Table II B, application no. 2, and being depicted in the same respective line as said B2923, and preferably the activity is increased non-targeted.

Accordingly, in one embodiment, the molecule which activity is to be increased in the process of the invention is the gene product with an activity as a “arginine exporter protein”, preferably it is the molecule of section (a) or (b) of this paragraph.

In particular, it was observed that in plants, especially in Arabidopsis thaliana, increasing or generating the activity of a gene product non-targeted with the activity of a “arginine exporter protein”, preferably being encoded by a gene comprising the nucleic acid sequence SEQ ID NO. 9333, preferably the coding region thereof, conferred the production of or the increase in glutamate compared with the wild type control.

The nucleic acid sequence of B2936 from Escherichia coli, e.g. as shown in the respective line in column 5 of Table I, application no. 2, has been published in Blattner et al., Science 277 (5331), 1453 (1997)). And the activity of the gene product thereof is the activity of b2936-protein.

Accordingly, in one embodiment, the process of the present invention for producing proline in a non-human organism, like a microorganism or a plant or a part thereof, comprises increasing or generating the activity of a gene product with the activity of a gene product conferring the activity of “b2936-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2936, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2936, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2936, or a functional equivalent or a homolog thereof as depicted in column 8 of Table II, application no. 2, preferably a homolog or functional equivalent as depicted in column 8 of Table II B, application no. 2, and being depicted in the same respective line as said B2936, and preferably the activity is increased non-targeted.

Accordingly, in one embodiment, the molecule which activity is to be increased in the process of the invention is the gene product with an activity as a “b2936-protein”, preferably it is the molecule of section (a) or (b) of this paragraph.

In particular, it was observed that in plants, especially in Arabidopsis thaliana, increasing or generating the activity of a gene product non-targeted with the activity of a “b2936-protein”, preferably being encoded by a gene comprising the nucleic acid sequence SEQ ID NO. 41442, preferably the coding region thereof, conferred the production of or the increase in proline compared with the wild type control.

The nucleic acid sequence of B2957 from Escherichia coli, e.g. as shown in the respective line in column 5 of Table I, application no. 2, has been published in Blattner et al., Science 277 (5331), 1453 (1997)). And the activity of the gene product thereof is the activity of L-asparaginase.

Accordingly, in one embodiment, the process of the present invention for producing proline in a non-human organism, like a microorganism or a plant or a part thereof, comprises increasing or generating the activity of a gene product with the activity of a gene product conferring the activity of “L-asparaginase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2957, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2957, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2957, or a functional equivalent or a homolog thereof as depicted in column 8 of Table II, application no. 2, preferably a homolog or functional equivalent as depicted in column 8 of Table II B, application no. 2, and being depicted in the same respective line as said B2957, and preferably the activity is increased non-targeted.

Accordingly, in one embodiment, the molecule which activity is to be increased in the process of the invention is the gene product with an activity as a “L-asparaginase”, preferably it is the molecule of section (a) or (b) of this paragraph.

In particular, it was observed that in plants, especially in Arabidopsis thaliana, increasing or generating the activity of a gene product non-targeted with the activity of a “L-asparaginase”, preferably being encoded by a gene comprising the nucleic acid sequence SEQ ID NO. 41499, preferably the coding region thereof, conferred the production of or the increase in proline compared with the wild type control.

The nucleic acid sequence of B2963 from Escherichia coli, e.g. as shown in the respective line in column 5 of Table I, application no. 2, has been published in Blattner et al., Science 277 (5331), 1453 (1997)). And the activity of the gene product thereof is the activity of murein transglycosylase.

Accordingly, in one embodiment, the process of the present invention for producing proline in a non-human organism, like a microorganism or a plant or a part thereof, comprises increasing or generating the activity of a gene product with the activity of a gene product conferring the activity of “murein transglycosylase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2963, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2963, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2963, or a functional equivalent or a homolog thereof as depicted in column 8 of Table II, application no. 2, preferably a homolog or functional equivalent as depicted in column 8 of Table II B, application no. 2, and being depicted in the same respective line as said B2963, and preferably the activity is increased plastidic.

Accordingly, in one embodiment, the molecule which activity is to be increased in the process of the invention is the gene product with an activity as a “murein transglycosylase”, preferably it is the molecule of section (a) or (b) of this paragraph.

In particular, it was observed that in plants, especially in Arabidopsis thaliana, increasing or generating the activity of a gene product plastidic with the activity of a “murein transglycosylase”, preferably being encoded by a gene comprising the nucleic acid sequence SEQ ID NO. 41732, preferably the coding region thereof, conferred the production of or the increase in proline compared with the wild type control.

The nucleic acid sequence of B2999 from Escherichia coli, e.g. as shown in the respective line in column 5 of Table I, application no. 2, has been published in Blattner et al., Science 277 (5331), 1453 (1997)). And the activity of the gene product thereof is the activity of b2999-protein.

Accordingly, in one embodiment, the process of the present invention for producing arginine in a non-human organism, like a microorganism or a plant or a part thereof, comprises increasing or generating the activity of a gene product with the activity of a gene product conferring the activity of “b2999-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B2999, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B2999, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B2999, or a functional equivalent or a homolog thereof as depicted in column 8 of Table II, application no. 2, preferably a homolog or functional equivalent as depicted in column 8 of Table II B, application no. 2, and being depicted in the same respective line as said B2999, and preferably the activity is increased non-targeted.

Accordingly, in one embodiment, the molecule which activity is to be increased in the process of the invention is the gene product with an activity as a “b2999-protein”, preferably it is the molecule of section (a) or (b) of this paragraph.

In particular, it was observed that in plants, especially in Arabidopsis thaliana, increasing or generating the activity of a gene product non-targeted with the activity of a “b2999-protein”, preferably being encoded by a gene comprising the nucleic acid sequence SEQ ID NO. 41797, preferably the coding region thereof, conferred the production of or the increase in arginine compared with the wild type control.

The nucleic acid sequence of B3064 from Escherichia coli, e.g. as shown in the respective line in column 5 of Table I, application no. 2, has been published in Blattner et al., Science 277 (5331), 1453 (1997)). And the activity of the gene product thereof is the activity of glycoprotease.

Accordingly, in one embodiment, the process of the present invention for producing proline in a non-human organism, like a microorganism or a plant or a part thereof, comprises increasing or generating the activity of a gene product with the activity of a gene product conferring the activity of “glycoprotease”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B3064, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B3064, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B3064, or a functional equivalent or a homolog thereof as depicted in column 8 of Table II, application no. 2, preferably a homolog or functional equivalent as depicted in column 8 of Table II B, application no. 2, and being depicted in the same respective line as said B3064, and preferably the activity is increased non-targeted.

Accordingly, in one embodiment, the molecule which activity is to be increased in the process of the invention is the gene product with an activity as a “glycoprotease”, preferably it is the molecule of section (a) or (b) of this paragraph.

In particular, it was observed that in plants, especially in Arabidopsis thaliana, increasing or generating the activity of a gene product non-targeted with the activity of a “glycoprotease”, preferably being encoded by a gene comprising the nucleic acid sequence SEQ ID NO. 42046, preferably the coding region thereof, conferred the production of or the increase in proline compared with the wild type control.

The nucleic acid sequence of B3121 from Escherichia coli, e.g. as shown in the respective line in column 5 of Table I, application no. 2, has been published in Blattner et al., Science 277 (5331), 1453 (1997)). And the activity of the gene product thereof is the activity of b3121-protein.

Accordingly, in one embodiment, the process of the present invention for producing arginine in a non-human organism, like a microorganism or a plant or a part thereof, comprises increasing or generating the activity of a gene product with the activity of a gene product conferring the activity of “b3121-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B3121, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B3121, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B3121, or a functional equivalent or a homolog thereof as depicted in column 8 of Table II, application no. 2, preferably a homolog or functional equivalent as depicted in column 8 of Table II B, application no. 2, and being depicted in the same respective line as said B3121, and preferably the activity is increased non-targeted.

Accordingly, in one embodiment, the molecule which activity is to be increased in the process of the invention is the gene product with an activity as a “b3121-protein”, preferably it is the molecule of section (a) or (b) of this paragraph.

In particular, it was observed that in plants, especially in Arabidopsis thaliana, increasing or generating the activity of a gene product non-targeted with the activity of a “b3121-protein”, preferably being encoded by a gene comprising the nucleic acid sequence SEQ ID NO. 42471, preferably the coding region thereof, conferred the production of or the increase in arginine compared with the wild type control.

The nucleic acid sequence of B3121 from Escherichia coli, e.g. as shown in the respective line in column 5 of Table I, application no. 2, has been published in Blattner et al., Science 277 (5331), 1453 (1997)). And the activity of the gene product thereof is the activity of b3121-protein.

Accordingly, in one embodiment, the process of the present invention for producing proline in a non-human organism, like a microorganism or a plant or a part thereof, comprises increasing or generating the activity of a gene product with the activity of a gene product conferring the activity of “b3121-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B3121, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B3121, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B3121, or a functional equivalent or a homolog thereof as depicted in column 8 of Table II, application no. 2, preferably a homolog or functional equivalent as depicted in column 8 of Table II B, application no. 2, and being depicted in the same respective line as said B3121, and preferably the activity is increased non-targeted.

Accordingly, in one embodiment, the molecule which activity is to be increased in the process of the invention is the gene product with an activity as a “b3121-protein”, preferably it is the molecule of section (a) or (b) of this paragraph.

In particular, it was observed that in plants, especially in Arabidopsis thaliana, increasing or generating the activity of a gene product non-targeted with the activity of a “b3121-protein”, preferably being encoded by a gene comprising the nucleic acid sequence SEQ ID NO. 42471, preferably the coding region thereof, conferred the production of or the increase in proline compared with the wild type control.

The nucleic acid sequence of B3151 from Escherichia coli, e.g. as shown in the respective line in column 5 of Table I, application no. 2, has been published in Blattner et al., Science 277 (5331), 1453 (1997)). And the activity of the gene product thereof is the activity of b3151-protein.

Accordingly, in one embodiment, the process of the present invention for producing proline in a non-human organism, like a microorganism or a plant or a part thereof, comprises increasing or generating the activity of a gene product with the activity of a gene product conferring the activity of “b3151-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B3151, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B3151, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B3151, or a functional equivalent or a homolog thereof as depicted in column 8 of Table II, application no. 2, preferably a homolog or functional equivalent as depicted in column 8 of Table II B, application no. 2, and being depicted in the same respective line as said B3151, and preferably the activity is increased non-targeted.

Accordingly, in one embodiment, the molecule which activity is to be increased in the process of the invention is the gene product with an activity as a “b3151-protein”, preferably it is the molecule of section (a) or (b) of this paragraph.

In particular, it was observed that in plants, especially in Arabidopsis thaliana, increasing or generating the activity of a gene product non-targeted with the activity of a “b3151-protein”, preferably being encoded by a gene comprising the nucleic acid sequence SEQ ID NO. 42477, preferably the coding region thereof, conferred the production of or the increase in proline compared with the wild type control.

The nucleic acid sequence of B3256 from Escherichia coli, e.g. as shown in the respective line in column 5 of Table I, application no. 2, has been published in Blattner et al., Science 277 (5331), 1453 (1997)). And the activity of the gene product thereof is the activity of acetyl CoA carboxylase.

Accordingly, in one embodiment, the process of the present invention for producing arginine in a non-human organism, like a microorganism or a plant or a part thereof, comprises increasing or generating the activity of a gene product with the activity of a gene product conferring the activity of “acetyl CoA carboxylase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B3256, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B3256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B3256, or a functional equivalent or a homolog thereof as depicted in column 8 of Table II, application no. 2, preferably a homolog or functional equivalent as depicted in column 8 of Table II B, application no. 2, and being depicted in the same respective line as said B3256, and preferably the activity is increased non-targeted.

Accordingly, in one embodiment, the molecule which activity is to be increased in the process of the invention is the gene product with an activity as a “acetyl CoA carboxylase”, preferably it is the molecule of section (a) or (b) of this paragraph.

In particular, it was observed that in plants, especially in Arabidopsis thaliana, increasing or generating the activity of a gene product non-targeted with the activity of a “acetyl CoA carboxylase”, preferably being encoded by a gene comprising the nucleic acid sequence SEQ ID NO. 9492, preferably the coding region thereof, conferred the production of or the increase in arginine compared with the wild type control.

The nucleic acid sequence of B3256 from Escherichia coli, e.g. as shown in the respective line in column 5 of Table I, application no. 2, has been published in Blattner et al., Science 277 (5331), 1453 (1997)). And the activity of the gene product thereof is the activity of acetyl CoA carboxylase.

Accordingly, in one embodiment, the process of the present invention for producing glutamate in a non-human organism, like a microorganism or a plant or a part thereof, comprises increasing or generating the activity of a gene product with the activity of a gene product conferring the activity of “acetyl CoA carboxylase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B3256, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B3256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B3256, or a functional equivalent or a homolog thereof as depicted in column 8 of Table II, application no. 2, preferably a homolog or functional equivalent as depicted in column 8 of Table II B, application no. 2, and being depicted in the same respective line as said B3256, and preferably the activity is increased non-targeted.

Accordingly, in one embodiment, the molecule which activity is to be increased in the process of the invention is the gene product with an activity as a “acetyl CoA carboxylase”, preferably it is the molecule of section (a) or (b) of this paragraph.

In particular, it was observed that in plants, especially in Arabidopsis thaliana, increasing or generating the activity of a gene product non-targeted with the activity of a “acetyl CoA carboxylase”, preferably being encoded by a gene comprising the nucleic acid sequence SEQ ID NO. 9492, preferably the coding region thereof, conferred the production of or the increase in glutamate compared with the wild type control.

The nucleic acid sequence of B3262 from Escherichia coli, e.g. as shown in the respective line in column 5 of Table I, application no. 2, has been published in Blattner et al., Science 277 (5331), 1453 (1997)). And the activity of the gene product thereof is the activity of methyltransferase.

Accordingly, in one embodiment, the process of the present invention for producing arginine in a non-human organism, like a microorganism or a plant or a part thereof, comprises increasing or generating the activity of a gene product with the activity of a gene product conferring the activity of “methyltransferase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B3262, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B3262, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B3262, or a functional equivalent or a homolog thereof as depicted in column 8 of Table II, application no. 2, preferably a homolog or functional equivalent as depicted in column 8 of Table II B, application no. 2, and being depicted in the same respective line as said B3262, and preferably the activity is increased non-targeted.

Accordingly, in one embodiment, the molecule which activity is to be increased in the process of the invention is the gene product with an activity as a “methyltransferase”, preferably it is the molecule of section (a) or (b) of this paragraph.

In particular, it was observed that in plants, especially in Arabidopsis thaliana, increasing or generating the activity of a gene product non-targeted with the activity of a “methyltransferase”, preferably being encoded by a gene comprising the nucleic acid sequence SEQ ID NO. 42502, preferably the coding region thereof, conferred the production of or the increase in arginine compared with the wild type control.

The nucleic acid sequence of B3262 from Escherichia coli, e.g. as shown in the respective line in column 5 of Table I, application no. 2, has been published in Blattner et al., Science 277 (5331), 1453 (1997)). And the activity of the gene product thereof is the activity of methyltransferase.

Accordingly, in one embodiment, the process of the present invention for producing glutamate in a non-human organism, like a microorganism or a plant or a part thereof, comprises increasing or generating the activity of a gene product with the activity of a gene product conferring the activity of “methyltransferase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B3262, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B3262, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B3262, or a functional equivalent or a homolog thereof as depicted in column 8 of Table II, application no. 2, preferably a homolog or functional equivalent as depicted in column 8 of Table II B, application no. 2, and being depicted in the same respective line as said B3262, and preferably the activity is increased non-targeted.

Accordingly, in one embodiment, the molecule which activity is to be increased in the process of the invention is the gene product with an activity as a “methyltransferase”, preferably it is the molecule of section (a) or (b) of this paragraph.

In particular, it was observed that in plants, especially in Arabidopsis thaliana, increasing or generating the activity of a gene product non-targeted with the activity of a “methyltransferase”, preferably being encoded by a gene comprising the nucleic acid sequence SEQ ID NO. 42502, preferably the coding region thereof, conferred the production of or the increase in glutamate compared with the wild type control.

The nucleic acid sequence of B3262 from Escherichia coli, e.g. as shown in the respective line in column 5 of Table I, application no. 2, has been published in Blattner et al., Science 277 (5331), 1453 (1997)). And the activity of the gene product thereof is the activity of methyltransferase.

Accordingly, in one embodiment, the process of the present invention for producing glutamine in a non-human organism, like a microorganism or a plant or a part thereof, comprises increasing or generating the activity of a gene product with the activity of a gene product conferring the activity of “methyltransferase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B3262, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B3262, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B3262, or a functional equivalent or a homolog thereof as depicted in column 8 of Table II, application no. 2, preferably a homolog or functional equivalent as depicted in column 8 of Table II B, application no. 2, and being depicted in the same respective line as said B3262, and preferably the activity is increased non-targeted.

Accordingly, in one embodiment, the molecule which activity is to be increased in the process of the invention is the gene product with an activity as a “methyltransferase”, preferably it is the molecule of section (a) or (b) of this paragraph.

In particular, it was observed that in plants, especially in Arabidopsis thaliana, increasing or generating the activity of a gene product non-targeted with the activity of a “methyltransferase”, preferably being encoded by a gene comprising the nucleic acid sequence SEQ ID NO. 42502, preferably the coding region thereof, conferred the production of or the increase in glutamine compared with the wild type control.

The nucleic acid sequence of B3262 from Escherichia coli, e.g. as shown in the respective line in column 5 of Table I, application no. 2, has been published in Blattner et al., Science 277 (5331), 1453 (1997)) d. And the activity of the gene product thereof is the activity of methyltransferase.

Accordingly, in one embodiment, the process of the present invention for producing proline in a non-human organism, like a microorganism or a plant or a part thereof, comprises increasing or generating the activity of a gene product with the activity of a gene product conferring the activity of “methyltransferase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B3262, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B3262, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B3262, or a functional equivalent or a homolog thereof as depicted in column 8 of Table II, application no. 2, preferably a homolog or functional equivalent as depicted in column 8 of Table II B, application no. 2, and being depicted in the same respective line as said B3262, and preferably the activity is increased non-targeted.

Accordingly, in one embodiment, the molecule which activity is to be increased in the process of the invention is the gene product with an activity as a “methyltransferase”, preferably it is the molecule of section (a) or (b) of this paragraph.

In particular, it was observed that in plants, especially in Arabidopsis thaliana, increasing or generating the activity of a gene product non-targeted with the activity of a “methyltransferase”, preferably being encoded by a gene comprising the nucleic acid sequence SEQ ID NO. 42502, preferably the coding region thereof, conferred the production of or the increase in proline compared with the wild type control.

The nucleic acid sequence of B3346 from Escherichia coli, e.g. as shown in the respective line in column 5 of Table I, application no. 2, has been published in Blattner et al., Science 277 (5331), 1453 (1997)). And the activity of the gene product thereof is the activity of b3346-protein.

Accordingly, in one embodiment, the process of the present invention for producing proline in a non-human organism, like a microorganism or a plant or a part thereof, comprises increasing or generating the activity of a gene product with the activity of a gene product conferring the activity of “b3346-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B3346, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B3346, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B3346, or a functional equivalent or a homolog thereof as depicted in column 8 of Table II, application no. 2, preferably a homolog or functional equivalent as depicted in column 8 of Table II B, application no. 2, and being depicted in the same respective line as said B3346, and preferably the activity is increased non-targeted.

Accordingly, in one embodiment, the molecule which activity is to be increased in the process of the invention is the gene product with an activity as a “b3346-protein”, preferably it is the molecule of section (a) or (b) of this paragraph.

In particular, it was observed that in plants, especially in Arabidopsis thaliana, increasing or generating the activity of a gene product non-targeted with the activity of a “b3346-protein”, preferably being encoded by a gene comprising the nucleic acid sequence SEQ ID NO. 10104, preferably the coding region thereof, conferred the production of or the increase in proline compared with the wild type control.

The nucleic acid sequence of B3410 from Escherichia coli, e.g. as shown in the respective line in column 5 of Table I, application no. 2, has been published in Blattner et al., Science 277 (5331), 1453 (1997)). And the activity of the gene product thereof is the activity of b3410-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “b3410-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B3410, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B3410, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B3410, or a functional equivalent or a homolog thereof as depicted in column 8 of Table II, application no. 2, preferably a homolog or functional equivalent as depicted in column 8 of Table II B, application no. 2, and being depicted in the same respective line as said B3410, and preferably the activity is increased non-targeted.

Accordingly, in one embodiment, the molecule which activity is to be increased in the process of the invention is the gene product with an activity as a “b3410-protein”, preferably it is the molecule of section (a) or (b) of this paragraph.

In particular, it was observed that in plants, especially in Arabidopsis thaliana, increasing or generating the activity of a gene product non-targeted with the activity of a “b3410-protein”, preferably being encoded by a gene comprising the nucleic acid sequence SEQ ID NO. 42559, preferably the coding region thereof, conferred the production of or the increase in glutamine compared with the wild type control.

The nucleic acid sequence of B3427 from Escherichia coli, e.g. as shown in the respective line in column 5 of Table I, application no. 2, has been published in Blattner et al., Science 277 (5331), 1453 (1997)). And the activity of the gene product thereof is the activity of B3427-protein.

Accordingly, in one embodiment, the process of the present invention for producing arginine in a non-human organism, like a microorganism or a plant or a part thereof, comprises increasing or generating the activity of a gene product with the activity of a gene product conferring the activity of “B3427-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B3427, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B3427, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B3427, or a functional equivalent or a homolog thereof as depicted in column 8 of Table II, application no. 2, preferably a homolog or functional equivalent as depicted in column 8 of Table II B, application no. 2, and being depicted in the same respective line as said B3427, and preferably the activity is increased non-targeted.

Accordingly, in one embodiment, the molecule which activity is to be increased in the process of the invention is the gene product with an activity as a “B3427-protein”, preferably it is the molecule of section (a) or (b) of this paragraph.

In particular, it was observed that in plants, especially in Arabidopsis thaliana, increasing or generating the activity of a gene product non-targeted with the activity of a “B3427-protein”, preferably being encoded by a gene comprising the nucleic acid sequence SEQ ID NO. 42579, preferably the coding region thereof, conferred the production of or the increase in arginine compared with the wild type control.

The nucleic acid sequence of B3509 from Escherichia coli, e.g. as shown in the respective line in column 5 of Table I, application no. 2, has been published in Blattner et al., Science 277 (5331), 1453 (1997)). And the activity of the gene product thereof is the activity of b3509-protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “b3509-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B3509, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B3509, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B3509, or a functional equivalent or a homolog thereof as depicted in column 8 of Table II, application no. 2, preferably a homolog or functional equivalent as depicted in column 8 of Table II B, application no. 2, and being depicted in the same respective line as said B3509, and preferably the activity is increased non-targeted.

Accordingly, in one embodiment, the molecule which activity is to be increased in the process of the invention is the gene product with an activity as a “b3509-protein”, preferably it is the molecule of section (a) or (b) of this paragraph.

In particular, it was observed that in plants, especially in Arabidopsis thaliana, increasing or generating the activity of a gene product non-targeted with the activity of a “b3509-protein”, preferably being encoded by a gene comprising the nucleic acid sequence SEQ ID NO. 42592, preferably the coding region thereof, conferred the production of or the increase in glutamate compared with the wild type control.

The nucleic acid sequence of B3572 from Escherichia coli, e.g. as shown in the respective line in column 5 of Table I, application no. 2, has been published in Blattner et al., Science 277 (5331), 1453 (1997)). And the activity of the gene product thereof is the activity of valine-pyruvate transaminase.

Accordingly, in one embodiment, the process of the present invention for producing proline in a non-human organism, like a microorganism or a plant or a part thereof, comprises increasing or generating the activity of a gene product with the activity of a gene product conferring the activity of “valine-pyruvate transaminase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B3572, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B3572, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B3572, or a functional equivalent or a homolog thereof as depicted in column 8 of Table II, application no. 2, preferably a homolog or functional equivalent as depicted in column 8 of Table II B, application no. 2, and being depicted in the same respective line as said B3572, and preferably the activity is increased plastidic.

Accordingly, in one embodiment, the molecule which activity is to be increased in the process of the invention is the gene product with an activity as a “valine-pyruvate transaminase”, preferably it is the molecule of section (a) or (b) of this paragraph.

In particular, it was observed that in plants, especially in Arabidopsis thaliana, increasing or generating the activity of a gene product plastidic with the activity of a “valine-pyruvate transaminase”, preferably being encoded by a gene comprising the nucleic acid sequence SEQ ID NO. 10172, preferably the coding region thereof, conferred the production of or the increase in proline compared with the wild type control.

The nucleic acid sequence of B3616 from Escherichia coli, e.g. as shown in the respective line in column 5 of Table I, application no. 2, has been published in Blattner et al., Science 277 (5331), 1453 (1997)). And the activity of the gene product thereof is the activity of threonine dehydrogenase.

Accordingly, in one embodiment, the process of the present invention for producing glutamine in a non-human organism, like a microorganism or a plant or a part thereof, comprises increasing or generating the activity of a gene product with the activity of a gene product conferring the activity of “threonine dehydrogenase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B3616, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B3616, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B3616, or a functional equivalent or a homolog thereof as depicted in column 8 of Table II, application no. 2, preferably a homolog or functional equivalent as depicted in column 8 of Table II B, application no. 2, and being depicted in the same respective line as said B3616, and preferably the activity is increased non-targeted.

Accordingly, in one embodiment, the molecule which activity is to be increased in the process of the invention is the gene product with an activity as a “threonine dehydrogenase”, preferably it is the molecule of section (a) or (b) of this paragraph.

In particular, it was observed that in plants, especially in Arabidopsis thaliana, increasing or generating the activity of a gene product non-targeted with the activity of a “threonine dehydrogenase”, preferably being encoded by a gene comprising the nucleic acid sequence SEQ ID NO. 42600, preferably the coding region thereof, conferred the production of or the increase in glutamine compared with the wild type control.

The nucleic acid sequence of B3616 from Escherichia coli, e.g. as shown in the respective line in column 5 of Table I, application no. 2, has been published in Blattner et al., Science 277 (5331), 1453 (1997)). And the activity of the gene product thereof is the activity of threonine dehydrogenase.

Accordingly, in one embodiment, the process of the present invention for producing proline in a non-human organism, like a microorganism or a plant or a part thereof, comprises increasing or generating the activity of a gene product with the activity of a gene product conferring the activity of “threonine dehydrogenase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B3616, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B3616, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B3616, or a functional equivalent or a homolog thereof as depicted in column 8 of Table II, application no. 2, preferably a homolog or functional equivalent as depicted in column 8 of Table II B, application no. 2, and being depicted in the same respective line as said B3616, and preferably the activity is increased non-targeted.

Accordingly, in one embodiment, the molecule which activity is to be increased in the process of the invention is the gene product with an activity as a “threonine dehydrogenase”, preferably it is the molecule of section (a) or (b) of this paragraph.

In particular, it was observed that in plants, especially in Arabidopsis thaliana, increasing or generating the activity of a gene product non-targeted with the activity of a “threonine dehydrogenase”, preferably being encoded by a gene comprising the nucleic acid sequence SEQ ID NO. 42600, preferably the coding region thereof, conferred the production of or the increase in proline compared with the wild type control.

The nucleic acid sequence of B3634 from Escherichia coli, e.g. as shown in the respective line in column 5 of Table I, application no. 2, has been published in Blattner et al., Science 277 (5331), 1453 (1997)). And the activity of the gene product thereof is the activity of phosphopantetheine adenylyltransferase.

Accordingly, in one embodiment, the process of the present invention for producing proline in a non-human organism, like a microorganism or a plant or a part thereof, comprises increasing or generating the activity of a gene product with the activity of a gene product conferring the activity of “phosphopantetheine adenylyltransferase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B3634, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B3634, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B3634, or a functional equivalent or a homolog thereof as depicted in column 8 of Table II, application no. 2, preferably a homolog or functional equivalent as depicted in column 8 of Table II B, application no. 2, and being depicted in the same respective line as said B3634, and preferably the activity is increased non-targeted.

Accordingly, in one embodiment, the molecule which activity is to be increased in the process of the invention is the gene product with an activity as a “phosphopantetheine adenylyltransferase”, preferably it is the molecule of section (a) or (b) of this paragraph.

In particular, it was observed that in plants, especially in Arabidopsis thaliana, increasing or generating the activity of a gene product non-targeted with the activity of a “phosphopantetheine adenylyltransferase”, preferably being encoded by a gene comprising the nucleic acid sequence SEQ ID NO. 42931, preferably the coding region thereof, conferred the production of or the increase in proline compared with the wild type control.

The nucleic acid sequence of B3732 from Escherichia coli, e.g. as shown in the respective line in column 5 of Table I, application no. 2, has been published in Blattner et al., Science 277 (5331), 1453 (1997)). And the activity of the gene product thereof is the activity of ATP synthase subunit beta.

Accordingly, in one embodiment, the process of the present invention for producing glutamine in a non-human organism, like a microorganism or a plant or a part thereof, comprises increasing or generating the activity of a gene product with the activity of a gene product conferring the activity of “ATP synthase subunit beta”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B3732, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B3732, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B3732, or a functional equivalent or a homolog thereof as depicted in column 8 of Table II, application no. 2, preferably a homolog or functional equivalent as depicted in column 8 of Table II B, application no. 2, and being depicted in the same respective line as said B3732, and preferably the activity is increased non-targeted.

Accordingly, in one embodiment, the molecule which activity is to be increased in the process of the invention is the gene product with an activity as a “ATP synthase subunit beta”, preferably it is the molecule of section (a) or (b) of this paragraph.

In particular, it was observed that in plants, especially in Arabidopsis thaliana, increasing or generating the activity of a gene product non-targeted with the activity of a “ATP synthase subunit beta”, preferably being encoded by a gene comprising the nucleic acid sequence SEQ ID NO. 43248, preferably the coding region thereof, conferred the production of or the increase in glutamine compared with the wild type control.

The nucleic acid sequence of B3793 from Escherichia coli, e.g. as shown in the respective line in column 5 of Table I, application no. 2, has been published in Blattner et al., Science 277 (5331), 1453 (1997)). And the activity of the gene product thereof is the activity of enterobacterial common antigen polymerase.

Accordingly, in one embodiment, the process of the present invention for producing arginine in a non-human organism, like a microorganism or a plant or a part thereof, comprises increasing or generating the activity of a gene product with the activity of a gene product conferring the activity of “enterobacterial common antigen polymerase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B3793, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B3793, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B3793, or a functional equivalent or a homolog thereof as depicted in column 8 of Table II, application no. 2, preferably a homolog or functional equivalent as depicted in column 8 of Table II B, application no. 2, and being depicted in the same respective line as said B3793, and preferably the activity is increased non-targeted.

Accordingly, in one embodiment, the molecule which activity is to be increased in the process of the invention is the gene product with an activity as a “enterobacterial common antigen polymerase”, preferably it is the molecule of section (a) or (b) of this paragraph.

In particular, it was observed that in plants, especially in Arabidopsis thaliana, increasing or generating the activity of a gene product non-targeted with the activity of a “enterobacterial common antigen polymerase”, preferably being encoded by a gene comprising the nucleic acid sequence SEQ ID NO. 43800, preferably the coding region thereof, conferred the production of or the increase in arginine compared with the wild type control.

The nucleic acid sequence of B3793 from Escherichia coli, e.g. as shown in the respective line in column 5 of Table I, application no. 2, has been published in Blattner et al., Science 277 (5331), 1453 (1997)). And the activity of the gene product thereof is the activity of enterobacterial common antigen polymerase.

Accordingly, in one embodiment, the process of the present invention for producing glutamate in a non-human organism, like a microorganism or a plant or a part thereof, comprises increasing or generating the activity of a gene product with the activity of a gene product conferring the activity of “enterobacterial common antigen polymerase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B3793, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B3793, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B3793, or a functional equivalent or a homolog thereof as depicted in column 8 of Table II, application no. 2, preferably a homolog or functional equivalent as depicted in column 8 of Table II B, application no. 2, and being depicted in the same respective line as said B3793, and preferably the activity is increased non-targeted.

Accordingly, in one embodiment, the molecule which activity is to be increased in the process of the invention is the gene product with an activity as a “enterobacterial common antigen polymerase”, preferably it is the molecule of section (a) or (b) of this paragraph.

In particular, it was observed that in plants, especially in Arabidopsis thaliana, increasing or generating the activity of a gene product non-targeted with the activity of a “enterobacterial common antigen polymerase”, preferably being encoded by a gene comprising the nucleic acid sequence SEQ ID NO. 43800, preferably the coding region thereof, conferred the production of or the increase in glutamate compared with the wild type control.

The nucleic acid sequence of B3813 from Escherichia coli, e.g. as shown in the respective line in column 5 of Table I, application no. 2, has been published in Blattner et al., Science 277 (5331), 1453 (1997)). And the activity of the gene product thereof is the activity of DNA helicase

Accordingly, in one embodiment, the process of the present invention for producing proline in a non-human organism, like a microorganism or a plant or a part thereof, comprises increasing or generating the activity of a gene product with the activity of a gene product conferring the activity of “DNA helicase II”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B3813, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B3813, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B3813, or a functional equivalent or a homolog thereof as depicted in column 8 of Table II, application no. 2, preferably a homolog or functional equivalent as depicted in column 8 of Table II B, application no. 2, and being depicted in the same respective line as said B3813, and preferably the activity is increased non-targeted.

Accordingly, in one embodiment, the molecule which activity is to be increased in the process of the invention is the gene product with an activity as a “DNA helicase II”, preferably it is the molecule of section (a) or (b) of this paragraph.

In particular, it was observed that in plants, especially in Arabidopsis thaliana, increasing or generating the activity of a gene product non-targeted with the activity of a “DNA helicase II”, preferably being encoded by a gene comprising the nucleic acid sequence SEQ ID NO. 43839, preferably the coding region thereof, conferred the production of or the increase in proline compared with the wild type control.

The nucleic acid sequence of B3814 from Escherichia coli, e.g. as shown in the respective line in column 5 of Table I, application no. 2, has been published in Blattner et al., Science 277 (5331), 1453 (1997)). And the activity of the gene product thereof is the activity of b3814*protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “b3814-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B3814, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B3814, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B3814, or a functional equivalent or a homolog thereof as depicted in column 8 of Table II, application no. 2, preferably a homolog or functional equivalent as depicted in column 8 of Table II B, application no. 2, and being depicted in the same respective line as said B3814, and preferably the activity is increased non-targeted.

Accordingly, in one embodiment, the molecule which activity is to be increased in the process of the invention is the gene product with an activity as a “b3814-protein”, preferably it is the molecule of section (a) or (b) of this paragraph.

In particular, it was observed that in plants, especially in Arabidopsis thaliana, increasing or generating the activity of a gene product non-targeted with the activity of a “b3814-protein”, preferably being encoded by a gene comprising the nucleic acid sequence SEQ ID NO. 44196, preferably the coding region thereof, conferred the production of or the increase in glutamine compared with the wild type control.

The nucleic acid sequence of B3817 from Escherichia coli, e.g. as shown in the respective line in column 5 of Table I, application no. 2, has been published in Blattner et al., Science 277 (5331), 1453 (1997)). And the activity of the gene product thereof is the activity of b3817-protein.

Accordingly, in one embodiment, the process of the present invention for producing proline in a non-human organism, like a microorganism or a plant or a part thereof, comprises increasing or generating the activity of a gene product with the activity of a gene product conferring the activity of “b3817-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B3817, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B3817, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B3817, or a functional equivalent or a homolog thereof as depicted in column 8 of Table II, application no. 2, preferably a homolog or functional equivalent as depicted in column 8 of Table II B, application no. 2, and being depicted in the same respective line as said B3817, and preferably the activity is increased non-targeted.

Accordingly, in one embodiment, the molecule which activity is to be increased in the process of the invention is the gene product with an activity as a “b3817-protein”, preferably it is the molecule of section (a) or (b) of this paragraph.

In particular, it was observed that in plants, especially in Arabidopsis thaliana, increasing or generating the activity of a gene product non-targeted with the activity of a “b3817-protein”, preferably being encoded by a gene comprising the nucleic acid sequence SEQ ID NO. 10708, preferably the coding region thereof, conferred the production of or the increase in proline compared with the wild type control.

The nucleic acid sequence of B3945 from Escherichia coli, e.g. as shown in the respective line in column 5 of Table I, application no. 2, has been published in Blattner et al., Science 277 (5331), 1453 (1997)). And the activity of the gene product thereof is the activity of glycerol dehydrogenase.

Accordingly, in one embodiment, the process of the present invention for producing glutamine in a non-human organism, like a microorganism or a plant or a part thereof, comprises increasing or generating the activity of a gene product with the activity of a gene product conferring the activity of “glycerol dehydrogenase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B3945, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B3945, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said

B3945, or a functional equivalent or a homolog thereof as depicted in column 8 of Table II, application no. 2, preferably a homolog or functional equivalent as depicted in column 8 of Table II B, application no. 2, and being depicted in the same respective line as said B3945, and preferably the activity is increased non-targeted.

Accordingly, in one embodiment, the molecule which activity is to be increased in the process of the invention is the gene product with an activity as a “glycerol dehydrogenase”, preferably it is the molecule of section (a) or (b) of this paragraph.

In particular, it was observed that in plants, especially in Arabidopsis thaliana, increasing or generating the activity of a gene product non-targeted with the activity of a “glycerol dehydrogenase”, preferably being encoded by a gene comprising the nucleic acid sequence SEQ ID NO. 44223, preferably the coding region thereof, conferred the production of or the increase in glutamine compared with the wild type control.

The nucleic acid sequence of B3989 from Escherichia coli, e.g. as shown in the respective line in column 5 of Table I, application no. 2, has been published in Blattner et al., Science 277 (5331), 1453 (1997)). And the activity of the gene product thereof is the activity of b3989-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “b3989-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B3989, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B3989, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B3989, or a functional equivalent or a homolog thereof as depicted in column 8 of Table II, application no. 2, preferably a homolog or functional equivalent as depicted in column 8 of Table II B, application no. 2, and being depicted in the same respective line as said B3989, and preferably the activity is increased non-targeted.

Accordingly, in one embodiment, the molecule which activity is to be increased in the process of the invention is the gene product with an activity as a “b3989-protein”, preferably it is the molecule of section (a) or (b) of this paragraph.

In particular, it was observed that in plants, especially in Arabidopsis thaliana, increasing or generating the activity of a gene product non-targeted with the activity of a “b3989-protein”, preferably being encoded by a gene comprising the nucleic acid sequence SEQ ID NO. 44372, preferably the coding region thereof, conferred the production of or the increase in glutamine compared with the wild type control.

The nucleic acid sequence of B4012 from Escherichia coli, e.g. as shown in the respective line in column 5 of Table I, application no. 2, has been published in Blattner et al., Science 277 (5331), 1453 (1997)). And the activity of the gene product thereof is the activity of acetyltransferase.

Accordingly, in one embodiment, the process of the present invention for producing glutamate in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B4012, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B4012, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B4012, or a functional equivalent or a homolog thereof as depicted in column 8 of Table II, application no. 2, preferably a homolog or functional equivalent as depicted in column 8 of Table II B, application no. 2, and being depicted in the same respective line as said B4012, and preferably the activity is increased non-targeted.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 44378, preferably the coding region thereof, conferred the production of or the increase in glutamate compared with the wild type control.

The nucleic acid sequence of B4029 from Escherichia coli, e.g. as shown in the respective line in column 5 of Table I, application no. 2, has been published in Blattner et al., Science 277 (5331), 1453 (1997)). And the activity of the gene product thereof is the activity of b4029-protein.

Accordingly, in one embodiment, the process of the present invention for producing arginine in a non-human organism, like a microorganism or a plant or a part thereof, comprises increasing or generating the activity of a gene product with the activity of a gene product conferring the activity of “b4029-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B4029, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B4029, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B4029, or a functional equivalent or a homolog thereof as depicted in column 8 of Table II, application no. 2, preferably a homolog or functional equivalent as depicted in column 8 of Table II B, application no. 2, and being depicted in the same respective line as said B4029, and preferably the activity is increased non-targeted.

Accordingly, in one embodiment, the molecule which activity is to be increased in the process of the invention is the gene product with an activity as a “b4029-protein”, preferably it is the molecule of section (a) or (b) of this paragraph.

In particular, it was observed that in plants, especially in Arabidopsis thaliana, increasing or generating the activity of a gene product non-targeted with the activity of a “b4029-protein”, preferably being encoded by a gene comprising the nucleic acid sequence SEQ ID NO. 10740, preferably the coding region thereof, conferred the production of or the increase in arginine compared with the wild type control.

The nucleic acid sequence of B4029 from Escherichia coli, e.g. as shown in the respective line in column 5 of Table I, application no. 2, has been published in Blattner et al., Science 277 (5331), 1453 (1997)). And the activity of the gene product thereof is the activity of b4029-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “b4029-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B4029, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B4029, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B4029, or a functional equivalent or a homolog thereof as depicted in column 8 of Table II, application no. 2, preferably a homolog or functional equivalent as depicted in column 8 of Table II B, application no. 2, and being depicted in the same respective line as said B4029, and preferably the activity is increased non-targeted.

Accordingly, in one embodiment, the molecule which activity is to be increased in the process of the invention is the gene product with an activity as a “b4029-protein”, preferably it is the molecule of section (a) or (b) of this paragraph.

In particular, it was observed that in plants, especially in Arabidopsis thaliana, increasing or generating the activity of a gene product non-targeted with the activity of a “b4029-protein”, preferably being encoded by a gene comprising the nucleic acid sequence SEQ ID NO. 10740, preferably the coding region thereof, conferred the production of or the increase in glutamine compared with the wild type control.

The nucleic acid sequence of B4029 from Escherichia coli, e.g. as shown in the respective line in column 5 of Table I, application no. 2, has been published in Blattner et al., Science 277 (5331), 1453 (1997)). And the activity of the gene product thereof is the activity of b4029-protein.

Accordingly, in one embodiment, the process of the present invention for producing proline in a non-human organism, like a microorganism or a plant or a part thereof, comprises increasing or generating the activity of a gene product with the activity of a gene product conferring the activity of “b4029-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B4029, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B4029, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B4029, or a functional equivalent or a homolog thereof as depicted in column 8 of Table II, application no. 2, preferably a homolog or functional equivalent as depicted in column 8 of Table II B, application no. 2, and being depicted in the same respective line as said B4029, and preferably the activity is increased non-targeted.

Accordingly, in one embodiment, the molecule which activity is to be increased in the process of the invention is the gene product with an activity as a “b4029-protein”, preferably it is the molecule of section (a) or (b) of this paragraph.

In particular, it was observed that in plants, especially in Arabidopsis thaliana, increasing or generating the activity of a gene product non-targeted with the activity of a “b4029-protein”, preferably being encoded by a gene comprising the nucleic acid sequence SEQ ID NO. 10740, preferably the coding region thereof, conferred the production of or the increase in proline compared with the wild type control.

The nucleic acid sequence of B4067 from Escherichia coli, e.g. as shown in the respective line in column 5 of Table I, application no. 2, has been published in Blattner et al., Science 277 (5331), 1453 (1997)). And the activity of the gene product thereof is the activity of cation/acetate symporter.

Accordingly, in one embodiment, the process of the present invention for producing arginine in a non-human organism, like a microorganism or a plant or a part thereof, comprises increasing or generating the activity of a gene product with the activity of a gene product conferring the activity of “cation/acetate symporter”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B4067, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B4067, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B4067, or a functional equivalent or a homolog thereof as depicted in column 8 of Table II, application no. 2, preferably a homolog or functional equivalent as depicted in column 8 of Table II B, application no. 2, and being depicted in the same respective line as said B4067, and preferably the activity is increased non-targeted.

Accordingly, in one embodiment, the molecule which activity is to be increased in the process of the invention is the gene product with an activity as a “cation/acetate symporter”, preferably it is the molecule of section (a) or (b) of this paragraph.

In particular, it was observed that in plants, especially in Arabidopsis thaliana, increasing or generating the activity of a gene product non-targeted with the activity of a “cation/acetate symporter”, preferably being encoded by a gene comprising the nucleic acid sequence SEQ ID NO. 44466, preferably the coding region thereof, conferred the production of or the increase in arginine compared with the wild type control.

The nucleic acid sequence of B4121 from Escherichia coli, e.g. as shown in the respective line in column 5 of Table I, application no. 2, has been published in Blattner et al., Science 277 (5331), 1453 (1997)). And the activity of the gene product thereof is the activity of b4121-protein.

Accordingly, in one embodiment, the process of the present invention for producing arginine in a non-human organism, like a microorganism or a plant or a part thereof, comprises increasing or generating the activity of a gene product with the activity of a gene product conferring the activity of “b4121-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B4121, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B4121, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B4121, or a functional equivalent or a homolog thereof as depicted in column 8 of Table II, application no. 2, preferably a homolog or functional equivalent as depicted in column 8 of Table II B, application no. 2, and being depicted in the same respective line as said B4121, and preferably the activity is increased non-targeted.

Accordingly, in one embodiment, the molecule which activity is to be increased in the process of the invention is the gene product with an activity as a “b4121-protein”, preferably it is the molecule of section (a) or (b) of this paragraph.

In particular, it was observed that in plants, especially in Arabidopsis thaliana, increasing or generating the activity of a gene product non-targeted with the activity of a “b4121-protein”, preferably being encoded by a gene comprising the nucleic acid sequence SEQ ID NO. 44609, preferably the coding region thereof, conferred the production of or the increase in arginine compared with the wild type control.

The nucleic acid sequence of B4121 from Escherichia coli, e.g. as shown in the respective line in column 5 of Table I, application no. 2, has been published in Blattner et al., Science 277 (5331), 1453 (1997)). And the activity of the gene product thereof is the activity of b4121-protein.

Accordingly, in one embodiment, the process of the present invention for producing proline in a non-human organism, like a microorganism or a plant or a part thereof, comprises increasing or generating the activity of a gene product with the activity of a gene product conferring the activity of “b4121-protein”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B4121, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B4121, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B4121, or a functional equivalent or a homolog thereof as depicted in column 8 of Table II, application no. 2, preferably a homolog or functional equivalent as depicted in column 8 of Table II B, application no. 2, and being depicted in the same respective line as said B4121, and preferably the activity is increased non-targeted.

Accordingly, in one embodiment, the molecule which activity is to be increased in the process of the invention is the gene product with an activity as a “b4121-protein”, preferably it is the molecule of section (a) or (b) of this paragraph.

In particular, it was observed that in plants, especially in Arabidopsis thaliana, increasing or generating the activity of a gene product non-targeted with the activity of a “b4121-protein”, preferably being encoded by a gene comprising the nucleic acid sequence SEQ ID NO. 44609, preferably the coding region thereof, conferred the production of or the increase in proline compared with the wild type control.

The nucleic acid sequence of B4129 from Escherichia coli, e.g. as shown in the respective line in column 5 of Table I, application no. 2, has been published in Blattner et al., Science 277 (5331), 1453 (1997)). And the activity of the gene product thereof is the activity of lysyl-tRNA synthetase.

Accordingly, in one embodiment, the process of the present invention for producing proline in a non-human organism, like a microorganism or a plant or a part thereof, comprises increasing or generating the activity of a gene product with the activity of a gene product conferring the activity of “lysyl-tRNA synthetase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B4129, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B4129, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B4129, or a functional equivalent or a homolog thereof as depicted in column 8 of Table II, application no. 2, preferably a homolog or functional equivalent as depicted in column 8 of Table II B, application no. 2, and being depicted in the same respective line as said B4129, and preferably the activity is increased non-targeted.

Accordingly, in one embodiment, the molecule which activity is to be increased in the process of the invention is the gene product with an activity as a “lysyl-tRNA synthetase”, preferably it is the molecule of section (a) or (b) of this paragraph.

In particular, it was observed that in plants, especially in Arabidopsis thaliana, increasing or generating the activity of a gene product non-targeted with the activity of a “lysyl-tRNA synthetase”, preferably being encoded by a gene comprising the nucleic acid sequence SEQ ID NO. 44662, preferably the coding region thereof, conferred the production of or the increase in proline compared with the wild type control.

The nucleic acid sequence of B4139 from Escherichia coli, e.g. as shown in the respective line in column 5 of Table I, application no. 2, has been published in Blattner et al., Science 277 (5331), 1453 (1997)). And the activity of the gene product thereof is the activity of aspartase.

Accordingly, in one embodiment, the process of the present invention for producing glutamine in a non-human organism, like a microorganism or a plant or a part thereof, comprises increasing or generating the activity of a gene product with the activity of a gene product conferring the activity of “aspartase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B4139, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B4139, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B4139, or a functional equivalent or a homolog thereof as depicted in column 8 of Table II, application no. 2, preferably a homolog or functional equivalent as depicted in column 8 of Table II B, application no. 2, and being depicted in the same respective line as said B4139, and preferably the activity is increased non-targeted.

Accordingly, in one embodiment, the molecule which activity is to be increased in the process of the invention is the gene product with an activity as a “aspartase”, preferably it is the molecule of section (a) or (b) of this paragraph.

In particular, it was observed that in plants, especially in Arabidopsis thaliana, increasing or generating the activity of a gene product non-targeted with the activity of a “aspartase”, preferably being encoded by a gene comprising the nucleic acid sequence SEQ ID NO. 45022, preferably the coding region thereof, conferred the production of or the increase in glutamine compared with the wild type control.

The nucleic acid sequence of B4256 from Escherichia coli, e.g. as shown in the respective line in column 5 of Table I, application no. 2, has been published in Blattner et al., Science 277 (5331), 1453 (1997)). And the activity of the gene product thereof is the activity of acetyltransferase.

Accordingly, in one embodiment, the process of the present invention for producing arginine in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B4256, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B4256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B4256, or a functional equivalent or a homolog thereof as depicted in column 8 of Table II, application no. 2, preferably a homolog or functional equivalent as depicted in column 8 of Table II B, application no. 2, and being depicted in the same respective line as said B4256, and preferably the activity is increased non-targeted.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 45321, preferably the coding region thereof, conferred the production of or the increase in arginine compared with the wild type control.

The nucleic acid sequence of B4256 from Escherichia coli, e.g. as shown in the respective line in column 5 of Table I, application no. 2, has been published in Blattner et al., Science 277 (5331), 1453 (1997)). And the activity of the gene product thereof is the activity of acetyltransferase.

Accordingly, in one embodiment, the process of the present invention for producing glutamine in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B4256, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B4256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B4256, or a functional equivalent or a homolog thereof as depicted in column 8 of Table II, application no. 2, preferably a homolog or functional equivalent as depicted in column 8 of Table II B, application no. 2, and being depicted in the same respective line as said B4256, and preferably the activity is increased non-targeted.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 45321, preferably the coding region thereof, conferred the production of or the increase in glutamine compared with the wild type control.

The nucleic acid sequence of B4256 from Escherichia coli, e.g. as shown in the respective line in column 5 of Table I, application no. 2, has been published in Blattner et al., Science 277 (5331), 1453 (1997)). And the activity of the gene product thereof is the activity of acetyltransferase.

Accordingly, in one embodiment, the process of the present invention for producing proline in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B4256, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B4256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B4256, or a functional equivalent or a homolog thereof as depicted in column 8 of Table II, application no. 2, preferably a homolog or functional equivalent as depicted in column 8 of Table II B, application no. 2, and being depicted in the same respective line as said B4256, and preferably the activity is increased non-targeted.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 45321, preferably the coding region thereof, conferred the production of or the increase in proline compared with the wild type control.

The nucleic acid sequence of B4321 from Escherichia coli, e.g. as shown in the respective line in column 5 of Table I, application no. 2, has been published in Blattner et al., Science 277 (5331), 1453 (1997)). And the activity of the gene product thereof is the activity of gluconate transport system permease 3.

Accordingly, in one embodiment, the process of the present invention for producing arginine in a non-human organism, like a microorganism or a plant or a part thereof, comprises increasing or generating the activity of a gene product with the activity of a gene product conferring the activity of “gluconate transport system permease 3”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B4321, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B4321, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B4321, or a functional equivalent or a homolog thereof as depicted in column 8 of Table II, application no. 2, preferably a homolog or functional equivalent as depicted in column 8 of Table II B, application no. 2, and being depicted in the same respective line as said B4321, and preferably the activity is increased non-targeted.

Accordingly, in one embodiment, the molecule which activity is to be increased in the process of the invention is the gene product with an activity as a “gluconate transport system permease 3”, preferably it is the molecule of section (a) or (b) of this paragraph.

In particular, it was observed that in plants, especially in Arabidopsis thaliana, increasing or generating the activity of a gene product non-targeted with the activity of a “gluconate transport system permease 3”, preferably being encoded by a gene comprising the nucleic acid sequence SEQ ID NO. 45394, preferably the coding region thereof, conferred the production of or the increase in arginine compared with the wild type control.

The nucleic acid sequence of B4384 from Escherichia coli, e.g. as shown in the respective line in column 5 of Table I, application no. 2, has been published in Blattner et al., Science 277 (5331), 1453 (1997)). And the activity of the gene product thereof is the activity of purinenucleoside phosphorylase.

Accordingly, in one embodiment, the process of the present invention for producing glutamate in a non-human organism, like a microorganism or a plant or a part thereof, comprises increasing or generating the activity of a gene product with the activity of a gene product conferring the activity of “purine-nucleoside phosphorylase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B4384, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B4384, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B4384, or a functional equivalent or a homolog thereof as depicted in column 8 of Table II, application no. 2, preferably a homolog or functional equivalent as depicted in column 8 of Table II B, application no. 2, and being depicted in the same respective line as said B4384, and preferably the activity is increased non-targeted.

Accordingly, in one embodiment, the molecule which activity is to be increased in the process of the invention is the gene product with an activity as a “purine-nucleoside phosphorylase”, preferably it is the molecule of section (a) or (b) of this paragraph.

In particular, it was observed that in plants, especially in Arabidopsis thaliana, increasing or generating the activity of a gene product non-targeted with the activity of a “purine-nucleoside phosphorylase”, preferably being encoded by a gene comprising the nucleic acid sequence SEQ ID NO. 45556, preferably the coding region thereof, conferred the production of or the increase in glutamate compared with the wild type control.

The nucleic acid sequence of B4384 from Escherichia coli, e.g. as shown in the respective line in column 5 of Table I, application no. 2, has been published in Blattner et al., Science 277 (5331), 1453 (1997)). And the activity of the gene product thereof is the activity of purinenucleoside phosphorylase.

Accordingly, in one embodiment, the process of the present invention for producing proline in a non-human organism, like a microorganism or a plant or a part thereof, comprises increasing or generating the activity of a gene product with the activity of a gene product conferring the activity of “purine-nucleoside phosphorylase”, especially from Escherichia coli or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said B4384, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said B4384, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said B4384, or a functional equivalent or a homolog thereof as depicted in column 8 of Table II, application no. 2, preferably a homolog or functional equivalent as depicted in column 8 of Table II B, application no. 2, and being depicted in the same respective line as said B4384, and preferably the activity is increased non-targeted.

Accordingly, in one embodiment, the molecule which activity is to be increased in the process of the invention is the gene product with an activity as a “purine-nucleoside phosphorylase”, preferably it is the molecule of section (a) or (b) of this paragraph.

In particular, it was observed that in plants, especially in Arabidopsis thaliana, increasing or generating the activity of a gene product non-targeted with the activity of a “purine-nucleoside phosphorylase”, preferably being encoded by a gene comprising the nucleic acid sequence SEQ ID NO. 45556, preferably the coding region thereof, conferred the production of or the increase in proline compared with the wild type control.

The nucleic acid sequence of C_pp004096192r from Physcomitrella patens, e.g. as shown in the respective line in column 5 of Table I, application no. 2 is unpublished. And the activity of the gene product thereof is the activity of thioredoxin.

Accordingly, in one embodiment, the process of the present invention for producing arginine in a non-human organism, like a microorganism or a plant or a part thereof, comprises increasing or generating the activity of a gene product with the activity of a gene product conferring the activity of “thioredoxin”, especially from Physcomitrella patens or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said C_pp004096192r, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said C_pp004096192r, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said C_pp004096192r, or a functional equivalent or a homolog thereof as depicted in column 8 of Table II, application no. 2, preferably a homolog or functional equivalent as depicted in column 8 of Table II B, application no. 2, and being depicted in the same respective line as said C_pp004096192r, and preferably the activity is increased non-targeted.

Accordingly, in one embodiment, the molecule which activity is to be increased in the process of the invention is the gene product with an activity as a “thioredoxin”, preferably it is the molecule of section (a) or (b) of this paragraph.

In particular, it was observed that in plants, especially in Arabidopsis thaliana, increasing or generating the activity of a gene product non-targeted with the activity of a “thioredoxin”, preferably being encoded by a gene comprising the nucleic acid sequence SEQ ID NO. 45757, preferably the coding region thereof, conferred the production of or the increase in arginine compared with the wild type control.

The nucleic acid sequence of GM02LC11114 from Glycine max, e.g. as shown in the respective line in column 5 of Table I, application no. 2 is unpublished. And the activity of the gene product thereof is the activity of GM02LC11114-protein.

Accordingly, in one embodiment, the process of the present invention for producing proline in a non-human organism, like a microorganism or a plant or a part thereof, comprises increasing or generating the activity of a gene product with the activity of a gene product conferring the activity of “GM02LC11114-protein”, especially from Glycine max or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said GM02LC11114, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said GM02LC11114, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said GM02LC11114, or a functional equivalent or a homolog thereof as depicted in column 8 of Table II, application no. 2, preferably a homolog or functional equivalent as depicted in column 8 of Table II B, application no. 2, and being depicted in the same respective line as said GM02LC11114, and preferably the activity is increased non-targeted.

Accordingly, in one embodiment, the molecule which activity is to be increased in the process of the invention is the gene product with an activity as a “GM02LC11114-protein”, preferably it is the molecule of section (a) or (b) of this paragraph.

In particular, it was observed that in plants, especially in Arabidopsis thaliana, increasing or generating the activity of a gene product non-targeted with the activity of a “GM02LC11114-protein”, preferably being encoded by a gene comprising the nucleic acid sequence SEQ ID NO. 45795, preferably the coding region thereof, conferred the production of or the increase in proline compared with the wild type control.

The nucleic acid sequence of GM02LC12622 from Glycine max, e.g. as shown in the respective line in column 5 of Table I, application no. 2 is unpublished. And the activity of the gene product thereof is the activity of glutaredoxin.

Accordingly, in one embodiment, the process of the present invention for producing glutamine in a non-human organism, like a microorganism or a plant or a part thereof, comprises increasing or generating the activity of a gene product with the activity of a gene product conferring the activity of “glutaredoxin”, especially from Glycine max or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said GM02LC12622, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said GM02LC12622, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said GM02LC12622, or a functional equivalent or a homolog thereof as depicted in column 8 of Table II, application no. 2, preferably a homolog or functional equivalent as depicted in column 8 of Table II B, application no. 2, and being depicted in the same respective line as said GM02LC12622, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 10811, preferably the coding region thereof, conferred the production of or the increase in glutamine compared with the wild type control.

The nucleic acid sequence of GM02LC15313 from Glycine max, e.g. as shown in the respective line in column 5 of Table I, application no. 2 is unpublished. And the activity of the gene product thereof is the activity of histone H2A.

Accordingly, in one embodiment, the process of the present invention for producing arginine in a non-human organism, like a microorganism or a plant or a part thereof, comprises increasing or generating the activity of a gene product with the activity of a gene product conferring the activity of “histone H2A”, especially from Glycine max or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said GM02LC15313, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said GM02LC15313, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said GM02LC15313, or a functional equivalent or a homolog thereof as depicted in column 8 of Table II, application no. 2, preferably a homolog or functional equivalent as depicted in column 8 of Table II B, application no. 2, and being depicted in the same respective line as said GM02LC15313, and preferably the activity is increased non-targeted.

Accordingly, in one embodiment, the molecule which activity is to be increased in the process of the invention is the gene product with an activity as a “histone H2A”, preferably it is the molecule of section (a) or (b) of this paragraph.

In particular, it was observed that in plants, especially in Arabidopsis thaliana, increasing or generating the activity of a gene product non-targeted with the activity of a “histone H2A”, preferably being encoded by a gene comprising the nucleic acid sequence SEQ ID NO. 45897, preferably the coding region thereof, conferred the production of or the increase in arginine compared with the wild type control.

The nucleic acid sequence of GM02LC15313 from Glycine max, e.g. as shown in the respective line in column 5 of Table I, application no. 2 is unpublished. And the activity of the gene product thereof is the activity of histone H2A.

Accordingly, in one embodiment, the process of the present invention for producing proline in a non-human organism, like a microorganism or a plant or a part thereof, comprises increasing or generating the activity of a gene product with the activity of a gene product conferring the activity of “histone H2A”, especially from Glycine max or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said GM02LC15313, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said GM02LC15313, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said GM02LC15313, or a functional equivalent or a homolog thereof as depicted in column 8 of Table II, application no. 2, preferably a homolog or functional equivalent as depicted in column 8 of Table II B, application no. 2, and being depicted in the same respective line as said GM02LC15313, and preferably the activity is increased non-targeted.

Accordingly, in one embodiment, the molecule which activity is to be increased in the process of the invention is the gene product with an activity as a “histone H2A”, preferably it is the molecule of section (a) or (b) of this paragraph.

In particular, it was observed that in plants, especially in Arabidopsis thaliana, increasing or generating the activity of a gene product non-targeted with the activity of a “histone H2A”, preferably being encoded by a gene comprising the nucleic acid sequence SEQ ID NO. 45897, preferably the coding region thereof, conferred the production of or the increase in proline compared with the wild type control.

The nucleic acid sequence of GM02LC17485 from Glycine max, e.g. as shown in the respective line in column 5 of Table I, application no. 2, is unpublished. And the activity of the gene product thereof is the activity of GM02LC17485-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “GM02LC17485-protein”, especially from Glycine max or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said GM02LC17485, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said GM02LC17485, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said GM02LC17485, or a functional equivalent or a homolog thereof as depicted in column 8 of Table II, application no. 2, preferably a homolog or functional equivalent as depicted in column 8 of Table II B, application no. 2, and being depicted in the same respective line as said GM02LC17485, and preferably the activity is increased non-targeted.

Accordingly, in one embodiment, the molecule which activity is to be increased in the process of the invention is the gene product with an activity as a “GM02LC17485-protein”, preferably it is the molecule of section (a) or (b) of this paragraph.

In particular, it was observed that in plants, especially in Arabidopsis thaliana, increasing or generating the activity of a gene product non-targeted with the activity of a “GM02LC17485-protein”, preferably being encoded by a gene comprising the nucleic acid sequence SEQ ID NO. 46405, preferably the coding region thereof, conferred the production of or the increase in glutamine compared with the wild type control.

The nucleic acid sequence of GM02LC17556 from Glycine max, e.g. as shown in the respective line in column 5 of Table I, application no. 2 is unpublished. And the activity of the gene product thereof is the activity of RNA binding protein.

Accordingly, in one embodiment, the process of the present invention for producing arginine in a non-human organism, like a microorganism or a plant or a part thereof, comprises increasing or generating the activity of a gene product with the activity of a gene product conferring the activity of “RNA binding protein”, especially from Glycine max or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said GM02LC17556, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said GM02LC17556, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said GM02LC17556, or a functional equivalent or a homolog thereof as depicted in column 8 of Table II, application no. 2, preferably a homolog or functional equivalent as depicted in column 8 of Table II B, application no. 2, and being depicted in the same respective line as said GM02LC17556, and preferably the activity is increased non-targeted,.

Accordingly, in one embodiment, the molecule which activity is to be increased in the process of the invention is the gene product with an activity as a “RNA binding protein”, preferably it is the molecule of section (a) or (b) of this paragraph.

In particular, it was observed that in plants, especially in Arabidopsis thaliana, increasing or generating the activity of a gene product non-targeted with the activity of a “RNA binding protein”, preferably being encoded by a gene comprising the nucleic acid sequence SEQ ID NO. 46515, preferably the coding region thereof, conferred the production of or the increase in arginine compared with the wild type control.

The nucleic acid sequence of GM02LC19289 from Glycine max, e.g. as shown in the respective line in column 5 of Table I, application no. 2 is unpublished. And the activity of the gene product thereof is the activity of transcription factor.

Accordingly, in one embodiment, the process of the present invention for producing proline in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said GM02LC19289, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said GM02LC19289, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said GM02LC19289, or a functional equivalent or a homolog thereof as depicted in column 8 of Table II, application no. 2, preferably a homolog or functional equivalent as depicted in column 8 of Table II B, application no. 2, and being depicted in the same respective line as said GM02LC19289, and preferably the activity is increased non-targeted.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 46751, preferably the coding region thereof, conferred the production of or the increase in proline compared with the wild type control.

The nucleic acid sequence of GM02LC44512 from Glycine max, e.g. as shown in the respective line in column 5 of Table I, application no. 2 is unpublished. And the activity of the gene product thereof is the activity of cyclin D.

Accordingly, in one embodiment, the process of the present invention for producing proline in a non-human organism, like a microorganism or a plant or a part thereof, comprises increasing or generating the activity of a gene product with the activity of a gene product conferring the activity of “cyclin D”, especially from Glycine max or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said GM02LC44512, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said GM02LC44512, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said GM02LC44512, or a functional equivalent or a homolog thereof as depicted in column 8 of Table II, application no. 2, preferably a homolog or functional equivalent as depicted in column 8 of Table II B, application no. 2, and being depicted in the same respective line as said GM02LC44512, and preferably the activity is increased non-targeted.

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 46850, preferably the coding region thereof, conferred the production of or the increase in proline compared with the wild type control.

The nucleic acid sequence of GM02LC46 from GLYCINE MAX, e.g. as shown in the respective line in column 5 of Table I, application no. 2 is unpublished. And the activity of the gene product thereof is the activity of GM02LC46-protein.

Accordingly, in one embodiment, the process of the present invention for producing proline in a non-human organism, like a microorganism or a plant or a part thereof, comprises increasing or generating the activity of a gene product with the activity of a gene product conferring the activity of “GM02LC46-protein”, especially from GLYCINE MAX or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said GM02LC46, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said GM02LC46, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said GM02LC46, or a functional equivalent or a homolog thereof as depicted in column 8 of Table II, application no. 2, preferably a homolog or functional equivalent as depicted in column 8 of Table II B, application no. 2, and being depicted in the same respective line as said GM02LC46, and preferably the activity is increased non-targeted.

Accordingly, in one embodiment, the molecule which activity is to be increased in the process of the invention is the gene product with an activity as a “GM02LC46-protein”, preferably it is the molecule of section (a) or (b) of this paragraph.

In particular, it was observed that in plants, especially in Arabidopsis thaliana, increasing or generating the activity of a gene product non-targeted with the activity of a “GM02LC46-protein”, preferably being encoded by a gene comprising the nucleic acid sequence SEQ ID NO. 47026, preferably the coding region thereof, conferred the production of or the increase in proline compared with the wild type control.

The nucleic acid sequence of GM02LC5744 from Glycine max, e.g. as shown in the respective line in column 5 of Table I, application no. 2 is unpublished. And the activity of the gene product thereof is the activity of GM02LC5744-protein.

Accordingly, in one embodiment, the process of the present invention for producing proline in a non-human organism, like a microorganism or a plant or a part thereof, comprises increasing or generating the activity of a gene product with the activity of a gene product conferring the activity of “GM02LC5744-protein”, especially from Glycine max or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said GM02LC5744, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said GM02LC5744, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said GM02LC5744, or a functional equivalent or a homolog thereof as depicted in column 8 of Table II, application no. 2, preferably a homolog or functional equivalent as depicted in column 8 of Table II B, application no. 2, and being depicted in the same respective line as said GM02LC5744, and preferably the activity is increased non-targeted.

Accordingly, in one embodiment, the molecule which activity is to be increased in the process of the invention is the gene product with an activity as a “GM02LC5744-protein”, preferably it is the molecule of section (a) or (b) of this paragraph.

In particular, it was observed that in plants, especially in Arabidopsis thaliana, increasing or generating the activity of a gene product non-targeted with the activity of a “GM02LC5744protein”, preferably being encoded by a gene comprising the nucleic acid sequence SEQ ID NO. 47076, preferably the coding region thereof, conferred the production of or the increase in proline compared with the wild type control.

The nucleic acid sequence of GM02LC6021 from Glycine max, e.g. as shown in the respective line in column 5 of Table I, application no. 2, is unpublished. And the activity of the gene product thereof is the activity of transcription factor.

Accordingly, in one embodiment, the process of the present invention for producing glutamate in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said GM02LC6021, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said GM02LC6021, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said GM02LC6021, or a functional equivalent or a homolog thereof as depicted in column 8 of Table II, application no. 2, preferably a homolog or functional equivalent as depicted in column 8 of Table II B, application no. 2, and being depicted in the same respective line as said GM02LC6021, and preferably the activity is increased non-targeted.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 47105, preferably the coding region thereof, conferred the production of or the increase in glutamate compared with the wild type control.

The nucleic acid sequence of NZ_AAAU02000016.150 from Azotobacter vinelandii, e.g. as shown in the respective line in column 5 of Table I, application no. 2, has been published by deposition of the sequences to the EMBL/GenBank/DDBJ databases under “Sequencing of the draft genome assembly of Azotobacter vinelandii AvOP.” in June 2005 by Copeland et al. And the activity of the gene product thereof is the activity of glutaredoxin.

Accordingly, in one embodiment, the process of the present invention for producing proline in a non-human organism, like a microorganism or a plant or a part thereof, comprises increasing or generating the activity of a gene product with the activity of a gene product conferring the activity of “glutaredoxin”, especially from Azotobacter vinelandii or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said NZ_AAAU02000016.150, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said NZ_AAAU02000016.150, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said NZ_AAAU02000016.150, or a functional equivalent or a homolog thereof as depicted in column 8 of Table II, application no. 2, preferably a homolog or functional equivalent as depicted in column 8 of Table II B, application no. 2, and being depicted in the same respective line as said NZ_AAAU02000016.150, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 47159, preferably the coding region thereof, conferred the production of or the increase in proline compared with the wild type control.

The nucleic acid sequence of S_pp015018333r from Physcomitrella patens, e.g. as shown in the respective line in column 5 of Table I, application no. 2 is unpublished. And the activity of the gene product thereof is the activity of s_pp015018333r-protein.

Accordingly, in one embodiment, the process of the present invention for producing arginine in a non-human organism, like a microorganism or a plant or a part thereof, comprises increasing or generating the activity of a gene product with the activity of a gene product conferring the activity of “s_pp015018333r-protein”, especially from Physcomitrella patens or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said S_pp015018333r, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said S_pp015018333r, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said S_pp015018333r, or a functional equivalent or a homolog thereof as depicted in column 8 of Table II, application no. 2, preferably a homolog or functional equivalent as depicted in column 8 of Table II B, application no. 2, and being depicted in the same respective line as said S_pp015018333r, and preferably the activity is increased non-targeted.

Accordingly, in one embodiment, the molecule which activity is to be increased in the process of the invention is the gene product with an activity as a “s_pp015018333r-protein”, preferably it is the molecule of section (a) or (b) of this paragraph.

In particular, it was observed that in plants, especially in Arabidopsis thaliana, increasing or generating the activity of a gene product non-targeted with the activity of a “s_pp015018333rprotein”, preferably being encoded by a gene comprising the nucleic acid sequence SEQ ID NO. 59851, preferably the coding region thereof, conferred the production of or the increase in arginine compared with the wild type control.

The nucleic acid sequence of S_pp015018333r from Physcomitrella patens, e.g. as shown in the respective line in column 5 of Table I, application no. 2 is unpublished. And the activity of the gene product thereof is the activity of s_pp015018333r-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “s_pp015018333r-protein”, especially from Physcomitrella patens or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said S_pp015018333r, or a functional equivalent or a homolog thereof as shown in column 8 of Table I, application no. 2, preferably the coding region thereof, particularly a homolog or functional equivalent as shown in column 8 of Table I B, application no. %%applicationnumber%%, and being depicted in the same respective line as said S_pp015018333r, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said S_pp015018333r, or a functional equivalent or a homolog thereof as depicted in column 8 of Table II, application no. 2, preferably a homolog or functional equivalent as depicted in column 8 of Table II B, application no. 2, and being depicted in the same respective line as said S_pp015018333r, and preferably the activity is increased non-targeted.

Accordingly, in one embodiment, the molecule which activity is to be increased in the process of the invention is the gene product with an activity as a “s_pp015018333r-protein”, preferably it is the molecule of section (a) or (b) of this paragraph.

In particular, it was observed that in plants, especially in Arabidopsis thaliana, increasing or generating the activity of a gene product non-targeted with the activity of a “s_pp015018333r-protein”, preferably being encoded by a gene comprising the nucleic acid sequence SEQ ID NO. 59851, preferably the coding region thereof, conferred the production of or the increase in glutamine compared with the wild type control.

The nucleic acid sequence of Sll0064 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of sll0064-protein.

Accordingly, in one embodiment, the process of the present invention for producing proline in a non-human organism, like a microorganism or a plant or a part thereof, comprises increasing or generating the activity of a gene product with the activity of a gene product conferring the activity of “sll0064-protein”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll0064, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll0064, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll0064, or a functional equivalent or a homolog thereof as depicted in column 8 of Table II, application no. 2, preferably a homolog or functional equivalent as depicted in column 8 of Table II B, application no. 2, and being depicted in the same respective line as said Sll0064, and preferably the activity is increased mitochondrial,.

Accordingly, in one embodiment, the molecule which activity is to be increased in the process of the invention is the gene product with an activity as a “sll0064-protein”, preferably it is the molecule of section (a) or (b) of this paragraph.
In particular, it was observed that in plants, especially in Arabidopsis thaliana, increasing or generating the activity of a gene product mitochondrial with the activity of a “sll0064-protein”, preferably being encoded by a gene comprising the nucleic acid sequence SEQ ID NO. 47526, preferably the coding region thereof, conferred the production of or the increase in proline compared with the wild type control.

The nucleic acid sequence of Sll0080 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of N-acetyl-gamma-glutamyl-phosphate reductase.

Accordingly, in one embodiment, the process of the present invention for producing glutamate in a non-human organism, like a microorganism or a plant or a part thereof, comprises increasing or generating the activity of a gene product with the activity of a gene product conferring the activity of “N-acetyl-gamma-glutamyl-phosphate reductase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll0080, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll0080, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll0080, or a functional equivalent or a homolog thereof as depicted in column 8 of Table II, application no. 2, preferably a homolog or functional equivalent as depicted in column 8 of Table II B, application no. 2, and being depicted in the same respective line as said Sll0080, and preferably the activity is increased plastidic.

Accordingly, in one embodiment, the molecule which activity is to be increased in the process of the invention is the gene product with an activity as a “N-acetyl-gamma-glutamyl-phosphate reductase”, preferably it is the molecule of section (a) or (b) of this paragraph.

In particular, it was observed that in plants, especially in Arabidopsis thaliana, increasing or generating the activity of a gene product plastidic with the activity of a “N-acetyl-gamma-glutamyl-phosphate reductase”, preferably being encoded by a gene comprising the nucleic acid sequence SEQ ID NO. 47566, preferably the coding region thereof, conferred the production of or the increase in glutamate compared with the wild type control.

The nucleic acid sequence of Sll0170 from Synechocystis sp., e.g. as shown in the respective line in column 5 of Table I, application no. 2, has been published in Synechocystis Nakamura et al., Nucleic Acids Res. 28(1), 72 (2000). And the activity of the gene product thereof is the activity of heat shock protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “heat shock protein”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll0170, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll0170, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll0170, or a functional equivalent or a homolog thereof as depicted in column 8 of Table II, application no. 2, preferably a homolog or functional equivalent as depicted in column 8 of Table II B, application no. 2, and being depicted in the same respective line as said Sll0170, and preferably the activity is increased non-targeted.

Accordingly, in one embodiment, the molecule which activity is to be increased in the process of the invention is the gene product with an activity as a “heat shock protein”, preferably it is the molecule of section (a) or (b) of this paragraph.

In particular, it was observed that in plants, especially in Arabidopsis thaliana, increasing or generating the activity of a gene product non-targeted with the activity of a “heat shock protein”, preferably being encoded by a gene comprising the nucleic acid sequence SEQ ID NO. 48138, preferably the coding region thereof, conferred the production of or the increase in glutamate compared with the wild type control.

The nucleic acid sequence of Sll0248 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of flavodoxin.

Accordingly, in one embodiment, the process of the present invention for producing arginine in a non-human organism, like a microorganism or a plant or a part thereof, comprises increasing or generating the activity of a gene product with the activity of a gene product conferring the activity of “flavodoxin”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll0248, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll0248, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll0248, or a functional equivalent or a homolog thereof as depicted in column 8 of Table II, application no. 2, preferably a homolog or functional equivalent as depicted in column 8 of Table II B, application no. 2, and being depicted in the same respective line as said Sll0248, and preferably the activity is increased plastidic.

Accordingly, in one embodiment, the molecule which activity is to be increased in the process of the invention is the gene product with an activity as a “flavodoxin”, preferably it is the molecule of section (a) or (b) of this paragraph.

In particular, it was observed that in plants, especially in Arabidopsis thaliana, increasing or generating the activity of a gene product plastidic with the activity of a “flavodoxin”, preferably being encoded by a gene comprising the nucleic acid sequence SEQ ID NO. 49143, preferably the coding region thereof, conferred the production of or the increase in arginine compared with the wild type control.

The nucleic acid sequence of Sll0250 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of phosphopantothenoylcysteinesynthetase/decarboxylase.

Accordingly, in one embodiment, the process of the present invention for producing proline in a non-human organism, like a microorganism or a plant or a part thereof, comprises increasing or generating the activity of a gene product with the activity of a gene product conferring the activity of “phosphopantothenoylcysteinesynthetase/decarboxylase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll0250, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll0250, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll0250, or a functional equivalent or a homolog thereof as depicted in column 8 of Table II, application no. 2, preferably a homolog or functional equivalent as depicted in column 8 of Table II B, application no. 2, and being depicted in the same respective line as said Sll0250, and preferably the activity is increased non-targeted.

Accordingly, in one embodiment, the molecule which activity is to be increased in the process of the invention is the gene product with an activity as a “phosphopantothenoylcysteinesynthetase/decarboxylase”, preferably it is the molecule of section (a) or (b) of this paragraph.

In particular, it was observed that in plants, especially in Arabidopsis thaliana, increasing or generating the activity of a gene product non-targeted with the activity of a “phosphopantothenoylcysteinesynthetase/decarboxylase”, preferably being encoded by a gene comprising the nucleic acid sequence SEQ ID NO. 49342, preferably the coding region thereof, conferred the production of or the increase in proline compared with the wild type control.

The nucleic acid sequence of Sll0254 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of sll0254-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “sll0254-protein”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll0254, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll0254, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll0254, or a functional equivalent or a homolog thereof as depicted in column 8 of Table II, application no. 2, preferably a homolog or functional equivalent as depicted in column 8 of Table II B, application no. 2, and being depicted in the same respective line as said Sll0254, and preferably the activity is increased mitochondrial.

Accordingly, in one embodiment, the molecule which activity is to be increased in the process of the invention is the gene product with an activity as a “sll0254-protein”, preferably it is the molecule of section (a) or (b) of this paragraph.

In particular, it was observed that in plants, especially in Arabidopsis thaliana, increasing or generating the activity of a gene product mitochondrial with the activity of a “sll0254-protein”, preferably being encoded by a gene comprising the nucleic acid sequence SEQ ID NO. 49800, preferably the coding region thereof, conferred the production of or the increase in glutamine compared with the wild type control.

The nucleic acid sequence of Sll0254 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of sll0254-protein.

Accordingly, in one embodiment, the process of the present invention for producing proline in a non-human organism, like a microorganism or a plant or a part thereof, comprises increasing or generating the activity of a gene product with the activity of a gene product conferring the activity of “sll0254-protein”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll0254, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll0254, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll0254, or a functional equivalent or a homolog thereof as depicted in column 8 of Table II, application no. 2, preferably a homolog or functional equivalent as depicted in column 8 of Table II B, application no. 2, and being depicted in the same respective line as said Sll0254, and preferably the activity is increased mitochondrial.

Accordingly, in one embodiment, the molecule which activity is to be increased in the process of the invention is the gene product with an activity as a “sll0254-protein”, preferably it is the molecule of section (a) or (b) of this paragraph.

In particular, it was observed that in plants, especially in Arabidopsis thaliana, increasing or generating the activity of a gene product mitochondrial with the activity of a “sll0254-protein”, preferably being encoded by a gene comprising the nucleic acid sequence SEQ ID NO. 49800, preferably the coding region thereof, conferred the production of or the increase in proline compared with the wild type control.

The nucleic acid sequence of Sll0290 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of polyphosphate kinase.

Accordingly, in one embodiment, the process of the present invention for producing proline in a non-human organism, like a microorganism or a plant or a part thereof, comprises increasing or generating the activity of a gene product with the activity of a gene product conferring the activity of “polyphosphate kinase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll0290, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll0290, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll0290, or a functional equivalent or a homolog thereof as depicted in column 8 of Table II, application no. 2, preferably a homolog or functional equivalent as depicted in column 8 of Table II B, application no. 2, and being depicted in the same respective line as said Sll0290, and preferably the activity is increased non-targeted.

Accordingly, in one embodiment, the molecule which activity is to be increased in the process of the invention is the gene product with an activity as a “polyphosphate kinase”, preferably it is the molecule of section (a) or (b) of this paragraph.

In particular, it was observed that in plants, especially in Arabidopsis thaliana, increasing or generating the activity of a gene product non-targeted with the activity of a “polyphosphate kinase”, preferably being encoded by a gene comprising the nucleic acid sequence SEQ ID NO. 49828, preferably the coding region thereof, conferred the production of or the increase in proline compared with the wild type control.

The nucleic acid sequence of Sll0354 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of sll0354-protein.

Accordingly, in one embodiment, the process of the present invention for producing proline in a non-human organism, like a microorganism or a plant or a part thereof, comprises increasing or generating the activity of a gene product with the activity of a gene product conferring the activity of “sll0354-protein”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll0354, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll0354, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll0354, or a functional equivalent or a homolog thereof as depicted in column 8 of Table II, application no. 2, preferably a homolog or functional equivalent as depicted in column 8 of Table II B, application no. 2, and being depicted in the same respective line as said Sll0354, and preferably the activity is increased non-targeted.

Accordingly, in one embodiment, the molecule which activity is to be increased in the process of the invention is the gene product with an activity as a “sll0354-protein”, preferably it is the molecule of section (a) or (b) of this paragraph.

In particular, it was observed that in plants, especially in Arabidopsis thaliana, increasing or generating the activity of a gene product non-targeted with the activity of a “sll0354-protein”, preferably being encoded by a gene comprising the nucleic acid sequence SEQ ID NO. 50070, preferably the coding region thereof, conferred the production of or the increase in proline compared with the wild type control.

The nucleic acid sequence of Sll0420 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of urease subunit.

Accordingly, in one embodiment, the process of the present invention for producing proline in a non-human organism, like a microorganism or a plant or a part thereof, comprises increasing or generating the activity of a gene product with the activity of a gene product conferring the activity of “urease subunit”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll0420, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll0420, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll0420, or a functional equivalent or a homolog thereof as depicted in column 8 of Table II, application no. 2, preferably a homolog or functional equivalent as depicted in column 8 of Table II B, application no. 2, and being depicted in the same respective line as said Sll0420, and preferably the activity is increased non-targeted.

Accordingly, in one embodiment, the molecule which activity is to be increased in the process of the invention is the gene product with an activity as a “urease subunit”, preferably it is the molecule of section (a) or (b) of this paragraph.

In particular, it was observed that in plants, especially in Arabidopsis thaliana, increasing or generating the activity of a gene product non-targeted with the activity of a “urease subunit”, preferably being encoded by a gene comprising the nucleic acid sequence SEQ ID NO. 50104, preferably the coding region thereof, conferred the production of or the increase in proline compared with the wild type control.

The nucleic acid sequence of Sll0521 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of NAD(P)H-quinone oxidoreductase subunit.

Accordingly, in one embodiment, the process of the present invention for producing arginine in a non-human organism, like a microorganism or a plant or a part thereof, comprises increasing or generating the activity of a gene product with the activity of a gene product conferring the activity of “NAD(P)H-quinone oxidoreductase subunit”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll0521, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll0521, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll0521, or a functional equivalent or a homolog thereof as depicted in column 8 of Table II, application no. 2, preferably a homolog or functional equivalent as depicted in column 8 of Table II B, application no. 2, and being depicted in the same respective line as said Sll0521, and preferably the activity is increased plastidic.

Accordingly, in one embodiment, the molecule which activity is to be increased in the process of the invention is the gene product with an activity as a “NAD(P)H-quinone oxidoreductase subunit”, preferably it is the molecule of section (a) or (b) of this paragraph.

In particular, it was observed that in plants, especially in Arabidopsis thaliana, increasing or generating the activity of a gene product plastidic with the activity of a “NAD(P)H-quinone oxidoreductase subunit”, preferably being encoded by a gene comprising the nucleic acid sequence SEQ ID NO. 50339, preferably the coding region thereof, conferred the production of or the increase in arginine compared with the wild type control.

The nucleic acid sequence of Sll0622 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of quinolinate synthetase.

Accordingly, in one embodiment, the process of the present invention for producing proline in a non-human organism, like a microorganism or a plant or a part thereof, comprises increasing or generating the activity of a gene product with the activity of a gene product conferring the activity of “quinolinate synthetase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll0622, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll0622, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll0622, or a functional equivalent or a homolog thereof as depicted in column 8 of Table II, application no. 2, preferably a homolog or functional equivalent as depicted in column 8 of Table II B, application no. 2, and being depicted in the same respective line as said Sll0622, and preferably the activity is increased mitochondrial.

Accordingly, in one embodiment, the molecule which activity is to be increased in the process of the invention is the gene product with an activity as a “quinolinate synthetase”, preferably it is the molecule of section (a) or (b) of this paragraph.

In particular, it was observed that in plants, especially in Arabidopsis thaliana, increasing or generating the activity of a gene product mitochondrial with the activity of a “quinolinate synthetase”, preferably being encoded by a gene comprising the nucleic acid sequence SEQ ID NO. 50713, preferably the coding region thereof, conferred the production of or the increase in proline compared with the wild type control.

he nucleic acid sequence of Sll0682 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of permease protein of phosphate ABC transporter.

Accordingly, in one embodiment, the process of the present invention for producing glutamate in a non-human organism, like a microorganism or a plant or a part thereof, comprises increasing or generating the activity of a gene product with the activity of a gene product conferring the activity of “permease protein of phosphate ABC transporter”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll0682, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll0682, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll0682, or a functional equivalent or a homolog thereof as depicted in column 8 of Table II, application no. 2, preferably a homolog or functional equivalent as depicted in column 8 of Table II B, application no. 2, and being depicted in the same respective line as said Sll0682, and preferably the activity is increased non-targeted.

Accordingly, in one embodiment, the molecule which activity is to be increased in the process of the invention is the gene product with an activity as a “permease protein of phosphate ABC transporter”, preferably it is the molecule of section (a) or (b) of this paragraph.

In particular, it was observed that in plants, especially in Arabidopsis thaliana, increasing or generating the activity of a gene product non-targeted with the activity of a “permease protein of phosphate ABC transporter”, preferably being encoded by a gene comprising the nucleic acid sequence SEQ ID NO. 50950, preferably the coding region thereof, conferred the production of or the increase in glutamate compared with the wild type control.

The nucleic acid sequence of Sll0682 from Synechocystis sp., e.g. as shown in the respective line in column 5 of Table I, application no. 2, has been published in Synechocystis Nakamura et al., Nucleic Acids Res. 28(1), 72 (2000). And the activity of the gene product thereof is the activity of permease protein of phosphate ABC transporter.

Accordingly, in one embodiment, the process of the present invention for producing proline in a non-human organism, like a microorganism or a plant or a part thereof, comprises increasing or generating the activity of a gene product with the activity of a gene product conferring the activity of “permease protein of phosphate ABC transporter”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll0682, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll0682, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll0682, or a functional equivalent or a homolog thereof as depicted in column 8 of Table II, application no. 2, preferably a homolog or functional equivalent as depicted in column 8 of Table II B, application no. 2, and being depicted in the same respective line as said Sll0682, and preferably the activity is increased non-targeted.

Accordingly, in one embodiment, the molecule which activity is to be increased in the process of the invention is the gene product with an activity as a “permease protein of phosphate ABC transporter”, preferably it is the molecule of section (a) or (b) of this paragraph.

In particular, it was observed that in plants, especially in Arabidopsis thaliana, increasing or generating the activity of a gene product non-targeted with the activity of a “permease protein of phosphate ABC transporter”, preferably being encoded by a gene comprising the nucleic acid sequence SEQ ID NO. 50950, preferably the coding region thereof, conferred the production of or the increase in proline compared with the wild type control.

The nucleic acid sequence of Sll0816 from Synechocystis sp., e.g. as shown in the respective line in column 5 of Table I, application no. 2, has been published in Synechocystis Nakamura et al., Nucleic Acids Res. 28(1), 72 (2000). And the activity of the gene product thereof is the activity of oxireductase.

Accordingly, in one embodiment, the process of the present invention for producing arginine in a non-human organism, like a microorganism or a plant or a part thereof, comprises increasing or generating the activity of a gene product with the activity of a gene product conferring the activity of “oxireductase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll0816, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll0816, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll0816, or a functional equivalent or a homolog thereof as depicted in column 8 of Table II, application no. 2, preferably a homolog or functional equivalent as depicted in column 8 of Table II B, application no. 2, and being depicted in the same respective line as said Sll0816, and preferably the activity is increased non-targeted.

Accordingly, in one embodiment, the molecule which activity is to be increased in the process of the invention is the gene product with an activity as a “oxireductase”, preferably it is the molecule of section (a) or (b) of this paragraph.

In particular, it was observed that in plants, especially in Arabidopsis thaliana, increasing or generating the activity of a gene product non-targeted with the activity of a “oxireductase”, preferably being encoded by a gene comprising the nucleic acid sequence SEQ ID NO. 51198, preferably the coding region thereof, conferred the production of or the increase in arginine compared with the wild type control.

The nucleic acid sequence of Sll0816 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of oxireductase.

Accordingly, in one embodiment, the process of the present invention for producing glutamate in a non-human organism, like a microorganism or a plant or a part thereof, comprises increasing or generating the activity of a gene product with the activity of a gene product conferring the activity of “oxireductase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll0816, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll0816, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll0816, or a functional equivalent or a homolog thereof as depicted in column 8 of Table II, application no. 2, preferably a homolog or functional equivalent as depicted in column 8 of Table II B, application no. 2, and being depicted in the same respective line as said Sll0816, and preferably the activity is increased non-targeted.

Accordingly, in one embodiment, the molecule which activity is to be increased in the process of the invention is the gene product with an activity as a “oxireductase”, preferably it is the molecule of section (a) or (b) of this paragraph.

In particular, it was observed that in plants, especially in Arabidopsis thaliana, increasing or generating the activity of a gene product non-targeted with the activity of a “oxireductase”, preferably being encoded by a gene comprising the nucleic acid sequence SEQ ID NO. 51198, preferably the coding region thereof, conferred the production of or the increase in glutamate compared with the wild type control.

The nucleic acid sequence of Sll0816 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of oxireductase.

Accordingly, in one embodiment, the process of the present invention for producing glutamine in a non-human organism, like a microorganism or a plant or a part thereof, comprises increasing or generating the activity of a gene product with the activity of a gene product conferring the activity of “oxireductase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll0816, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll0816, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll0816, or a functional equivalent or a homolog thereof as depicted in column 8 of Table II, application no. 2, preferably a homolog or functional equivalent as depicted in column 8 of Table II B, application no. 2, and being depicted in the same respective line as said Sll0816, and preferably the activity is increased non-targeted.

Accordingly, in one embodiment, the molecule which activity is to be increased in the process of the invention is the gene product with an activity as a “oxireductase”, preferably it is the molecule of section (a) or (b) of this paragraph.

In particular, it was observed that in plants, especially in Arabidopsis thaliana, increasing or generating the activity of a gene product non-targeted with the activity of a “oxireductase”, preferably being encoded by a gene comprising the nucleic acid sequence SEQ ID NO. 51198, preferably the coding region thereof, conferred the production of or the increase in glutamine compared with the wild type control.

The nucleic acid sequence of Sll0891 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of malate dehydrogenase.

Accordingly, in one embodiment, the process of the present invention for producing arginine in a non-human organism, like a microorganism or a plant or a part thereof, comprises increasing or generating the activity of a gene product with the activity of a gene product conferring the activity of “malate dehydrogenase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll0891, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll0891, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll0891, or a functional equivalent or a homolog thereof as depicted in column 8 of Table II, application no. 2, preferably a homolog or functional equivalent as depicted in column 8 of Table II B, application no. 2, and being depicted in the same respective line as said Sll0891, and preferably the activity is increased plastidic.

Accordingly, in one embodiment, the molecule which activity is to be increased in the process of the invention is the gene product with an activity as a “malate dehydrogenase”, preferably it is the molecule of section (a) or (b) of this paragraph.

In particular, it was observed that in plants, especially in Arabidopsis thaliana, increasing or generating the activity of a gene product plastidic with the activity of a “malate dehydrogenase”, preferably being encoded by a gene comprising the nucleic acid sequence SEQ ID NO. 51268, preferably the coding region thereof, conferred the production of or the increase in arginine compared with the wild type control.

The nucleic acid sequence of Sll0891 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of malate dehydrogenase.

Accordingly, in one embodiment, the process of the present invention for producing glutamine in a non-human organism, like a microorganism or a plant or a part thereof, comprises increasing or generating the activity of a gene product with the activity of a gene product conferring the activity of “malate dehydrogenase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll0891, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll0891, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll0891, or a functional equivalent or a homolog thereof as depicted in column 8 of Table II, application no. 2, preferably a homolog or functional equivalent as depicted in column 8 of Table II B, application no. 2, and being depicted in the same respective line as said Sll0891, and preferably the activity is increased plastidic.

Accordingly, in one embodiment, the molecule which activity is to be increased in the process of the invention is the gene product with an activity as a “malate dehydrogenase”, preferably it is the molecule of section (a) or (b) of this paragraph.

In particular, it was observed that in plants, especially in Arabidopsis thaliana, increasing or generating the activity of a gene product plastidic with the activity of a “malate dehydrogenase”, preferably being encoded by a gene comprising the nucleic acid sequence SEQ ID NO. 51268, preferably the coding region thereof, conferred the production of or the increase in glutamine compared with the wild type control.

The nucleic acid sequence of Sll0891 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of malate dehydrogenase.

Accordingly, in one embodiment, the process of the present invention for producing proline in a non-human organism, like a microorganism or a plant or a part thereof, comprises increasing or generating the activity of a gene product with the activity of a gene product conferring the activity of “malate dehydrogenase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll0891, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll0891, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll0891, or a functional equivalent or a homolog thereof as depicted in column 8 of Table II, application no. 2, preferably a homolog or functional equivalent as depicted in column 8 of Table II B, application no. 2, and being depicted in the same respective line as said Sll0891, and preferably the activity is increased plastidic.

Accordingly, in one embodiment, the molecule which activity is to be increased in the process of the invention is the gene product with an activity as a “malate dehydrogenase”, preferably it is the molecule of section (a) or (b) of this paragraph.

In particular, it was observed that in plants, especially in Arabidopsis thaliana, increasing or generating the activity of a gene product plastidic with the activity of a “malate dehydrogenase”, preferably being encoded by a gene comprising the nucleic acid sequence SEQ ID NO. 51268, preferably the coding region thereof, conferred the production of or the increase in proline compared with the wild type control.

The nucleic acid sequence of Sll0901 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of phosphoribosylaminoimidazole carboxylase catalytic subunit.

Accordingly, in one embodiment, the process of the present invention for producing proline in a non-human organism, like a microorganism or a plant or a part thereof, comprises increasing or generating the activity of a gene product with the activity of a gene product conferring the activity of “phosphoribosylaminoimidazole carboxylase catalytic subunit”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll0901, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll0901, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll0901, or a functional equivalent or a homolog thereof as depicted in column 8 of Table II, application no. 2, preferably a homolog or functional equivalent as depicted in column 8 of Table II B, application no. 2, and being depicted in the same respective line as said Sll0901, and preferably the activity is increased mitochondrial.

Accordingly, in one embodiment, the molecule which activity is to be increased in the process of the invention is the gene product with an activity as a “phosphoribosylaminoimidazole carboxylase catalytic subunit”, preferably it is the molecule of section (a) or (b) of this paragraph.

In particular, it was observed that in plants, especially in Arabidopsis thaliana, increasing or generating the activity of a gene product mitochondrial with the activity of a “phosphoribosylaminoimidazole carboxylase catalytic subunit”, preferably being encoded by a gene comprising the nucleic acid sequence SEQ ID NO. 51632, preferably the coding region thereof, conferred the production of or the increase in proline compared with the wild type control.

The nucleic acid sequence of Sll0934 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of 3-deoxy-7-phosphoheptulonate synthase.

Accordingly, in one embodiment, the process of the present invention for producing glutamine in a non-human organism, like a microorganism or a plant or a part thereof, comprises increasing or generating the activity of a gene product with the activity of a gene product conferring the activity of “3-deoxy-7-phosphoheptulonate synthase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll0934, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll0934, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll0934, or a functional equivalent or a homolog thereof as depicted in column 8 of Table II, application no. 2, preferably a homolog or functional equivalent as depicted in column 8 of Table II B, application no. 2, and being depicted in the same respective line as said Sll0934, and preferably the activity is increased mitochondrial.

Accordingly, in one embodiment, the molecule which activity is to be increased in the process of the invention is the gene product with an activity as a “3-deoxy-7-phosphoheptulonate synthase”, preferably it is the molecule of section (a) or (b) of this paragraph.

In particular, it was observed that in plants, especially in Arabidopsis thaliana, increasing or generating the activity of a gene product mitochondrial with the activity of a “3-deoxy-7-phosphoheptulonate synthase”, preferably being encoded by a gene comprising the nucleic acid sequence SEQ ID NO. 52246, preferably the coding region thereof, conferred the production of or the increase in glutamine compared with the wild type control.

The nucleic acid sequence of Sll0945 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of glycogen synthase.

Accordingly, in one embodiment, the process of the present invention for producing proline in a non-human organism, like a microorganism or a plant or a part thereof, comprises increasing or generating the activity of a gene product with the activity of a gene product conferring the activity of “glycogen synthase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll0945, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll0945, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll0945, or a functional equivalent or a homolog thereof as depicted in column 8 of Table II, application no. 2, preferably a homolog or functional equivalent as depicted in column 8 of Table II B, application no. 2, and being depicted in the same respective line as said Sll0945, and preferably the activity is increased non-targeted.

Accordingly, in one embodiment, the molecule which activity is to be increased in the process of the invention is the gene product with an activity as a “glycogen synthase”, preferably it is the molecule of section (a) or (b) of this paragraph.

In particular, it was observed that in plants, especially in Arabidopsis thaliana, increasing or generating the activity of a gene product non-targeted with the activity of a “glycogen synthase”, preferably being encoded by a gene comprising the nucleic acid sequence SEQ ID NO. 52364, preferably the coding region thereof, conferred the production of or the increase in proline compared with the wild type control.

The nucleic acid sequence of Sll1031 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of carbon dioxide concentrating mechanism protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “carbon dioxide concentrating mechanism protein”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll1031, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll1031, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll1031, or a functional equivalent or a homolog thereof as depicted in column 8 of Table II, application no. 2, preferably a homolog or functional equivalent as depicted in column 8 of Table II B, application no. 2, and being depicted in the same respective line as said Sll1031, and preferably the activity is increased mitochondrial.

Accordingly, in one embodiment, the molecule which activity is to be increased in the process of the invention is the gene product with an activity as a “carbon dioxide concentrating mechanism protein”, preferably it is the molecule of section (a) or (b) of this paragraph.

In particular, it was observed that in plants, especially in Arabidopsis thaliana, increasing or generating the activity of a gene product mitochondrial with the activity of a “carbon dioxide concentrating mechanism protein”, preferably being encoded by a gene comprising the nucleic acid sequence SEQ ID NO. 52634, preferably the coding region thereof, conferred the production of or the increase in glutamate compared with the wild type control.

The nucleic acid sequence of Sll1031 from Synechocystis sp., e.g. as shown in the respective line in column 5 of Table I, application no. 2, has been published in Synechocystis Nakamura et al., Nucleic Acids Res. 28(1), 72 (2000). And the activity of the gene product thereof is the activity of carbon dioxide concentrating mechanism protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “carbon dioxide concentrating mechanism protein”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll1031, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll1031, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll1031, or a functional equivalent or a homolog thereof as depicted in column 8 of Table II, application no. 2, preferably a homolog or functional equivalent as depicted in column 8 of Table II B, application no. 2, and being depicted in the same respective line as said Sll1031, and preferably the activity is increased mitochondrial.

Accordingly, in one embodiment, the molecule which activity is to be increased in the process of the invention is the gene product with an activity as a “carbon dioxide concentrating mechanism protein”, preferably it is the molecule of section (a) or (b) of this paragraph.

In particular, it was observed that in plants, especially in Arabidopsis thaliana, increasing or generating the activity of a gene product mitochondrial with the activity of a “carbon dioxide concentrating mechanism protein”, preferably being encoded by a gene comprising the nucleic acid sequence SEQ ID NO. 52634, preferably the coding region thereof, conferred the production of or the increase in glutamine compared with the wild type control.

The nucleic acid sequence of Sll1031 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of carbon dioxide concentrating mechanism protein.

Accordingly, in one embodiment, the process of the present invention for producing proline in a non-human organism, like a microorganism or a plant or a part thereof, comprises increasing or generating the activity of a gene product with the activity of a gene product conferring the activity of “carbon dioxide concentrating mechanism protein”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll1031, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll1031, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll1031, or a functional equivalent or a homolog thereof as depicted in column 8 of Table II, application no. 2, preferably a homolog or functional equivalent as depicted in column 8 of Table II B, application no. 2, and being depicted in the same respective line as said Sll1031, and preferably the activity is increased mitochondrial.

Accordingly, in one embodiment, the molecule which activity is to be increased in the process of the invention is the gene product with an activity as a “carbon dioxide concentrating mechanism protein”, preferably it is the molecule of section (a) or (b) of this paragraph.

In particular, it was observed that in plants, especially in Arabidopsis thaliana, increasing or generating the activity of a gene product mitochondrial with the activity of a “carbon dioxide concentrating mechanism protein”, preferably being encoded by a gene comprising the nucleic acid sequence SEQ ID NO. 52634, preferably the coding region thereof, conferred the production of or the increase in proline compared with the wild type control.

The nucleic acid sequence of Sll1056 from Synechocystis sp., e.g. as shown in the respective line in column 5 of Table I, application no. 2, has been published in Synechocystis Nakamura et al., Nucleic Acids Res. 28(1), 72 (2000). And the activity of the gene product thereof is the activity of phosphoribosylformyl glycinamidine synthase subunit.

Accordingly, in one embodiment, the process of the present invention for producing proline in a non-human organism, like a microorganism or a plant or a part thereof, comprises increasing or generating the activity of a gene product with the activity of a gene product conferring the activity of “phosphoribosylformyl glycinamidine synthase subunit”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll1056, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll1056, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll1056, or a functional equivalent or a homolog thereof as depicted in column 8 of Table II, application no. 2, preferably a homolog or functional equivalent as depicted in column 8 of Table II B, application no. 2, and being depicted in the same respective line as said Sll1056, and preferably the activity is increased plastidic.

Accordingly, in one embodiment, the molecule which activity is to be increased in the process of the invention is the gene product with an activity as a “phosphoribosylformyl glycinamidine synthase subunit”, preferably it is the molecule of section (a) or (b) of this paragraph.

In particular, it was observed that in plants, especially in Arabidopsis thaliana, increasing or generating the activity of a gene product plastidic with the activity of a “phosphoribosylformyl glycinamidine synthase subunit”, preferably being encoded by a gene comprising the nucleic acid sequence SEQ ID NO. 52660, preferably the coding region thereof, conferred the production of or the increase in proline compared with the wild type control.

The nucleic acid sequence of Sll1185 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of coproporphyrinogen oxidase.

Accordingly, in one embodiment, the process of the present invention for producing glutamate in a non-human organism, like a microorganism or a plant or a part thereof, comprises increasing or generating the activity of a gene product with the activity of a gene product conferring the activity of “coproporphyrinogen oxidase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll1185, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll1185, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll1185, or a functional equivalent or a homolog thereof as depicted in column 8 of Table II, application no. 2, preferably a homolog or functional equivalent as depicted in column 8 of Table II B, application no. 2, and being depicted in the same respective line as said Sll1185, and preferably the activity is increased non-targeted.

Accordingly, in one embodiment, the molecule which activity is to be increased in the process of the invention is the gene product with an activity as a “coproporphyrinogen oxidase”, preferably it is the molecule of section (a) or (b) of this paragraph.

In particular, it was observed that in plants, especially in Arabidopsis thaliana, increasing or generating the activity of a gene product non-targeted with the activity of a “coproporphyrinogen oxidase”, preferably being encoded by a gene comprising the nucleic acid sequence SEQ ID NO. 53189, preferably the coding region thereof, conferred the production of or the increase in glutamate compared with the wild type control.

The nucleic acid sequence of Sll1393 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of glycogen (starch) synthase.

Accordingly, in one embodiment, the process of the present invention for producing glutamine in a non-human organism, like a microorganism or a plant or a part thereof, comprises increasing or generating the activity of a gene product with the activity of a gene product conferring the activity of “glycogen (starch) synthase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll1393, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll1393, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll1393, or a functional equivalent or a homolog thereof as depicted in column 8 of Table II, application no. 2, preferably a homolog or functional equivalent as depicted in column 8 of Table II B, application no. 2, and being depicted in the same respective line as said Sll1393, and preferably the activity is increased non-targeted.

Accordingly, in one embodiment, the molecule which activity is to be increased in the process of the invention is the gene product with an activity as a “glycogen (starch) synthase”, preferably it is the molecule of section (a) or (b) of this paragraph.

In particular, it was observed that in plants, especially in Arabidopsis thaliana, increasing or generating the activity of a gene product non-targeted with the activity of a “glycogen (starch) synthase”, preferably being encoded by a gene comprising the nucleic acid sequence SEQ ID NO. 53456, preferably the coding region thereof, conferred the production of or the increase in glutamine compared with the wild type control.

The nucleic acid sequence of Sll1393 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of glycogen (starch) synthase.

Accordingly, in one embodiment, the process of the present invention for producing proline in a non-human organism, like a microorganism or a plant or a part thereof, comprises increasing or generating the activity of a gene product with the activity of a gene product conferring the activity of “glycogen (starch) synthase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll1393, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll1393, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll1393, or a functional equivalent or a homolog thereof as depicted in column 8 of Table II, application no. 2, preferably a homolog or functional equivalent as depicted in column 8 of Table II B, application no. 2, and being depicted in the same respective line as said Sll1393, and preferably the activity is increased non-targeted.

Accordingly, in one embodiment, the molecule which activity is to be increased in the process of the invention is the gene product with an activity as a “glycogen (starch) synthase”, preferably it is the molecule of section (a) or (b) of this paragraph.

In particular, it was observed that in plants, especially in Arabidopsis thaliana, increasing or generating the activity of a gene product non-targeted with the activity of a “glycogen (starch) synthase”, preferably being encoded by a gene comprising the nucleic acid sequence SEQ ID NO. 53456, preferably the coding region thereof, conferred the production of or the increase in proline compared with the wild type control.

The nucleic acid sequence of Sll1441 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of fatty acid desaturase.

Accordingly, in one embodiment, the process of the present invention for producing glutamine in a non-human organism, like a microorganism or a plant or a part thereof, comprises increasing or generating the activity of a gene product with the activity of a gene product conferring the activity of “fatty acid desaturase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll1441, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll1441, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll1441, or a functional equivalent or a homolog thereof as depicted in column 8 of Table II, application no. 2, preferably a homolog or functional equivalent as depicted in column 8 of Table II B, application no. 2, and being depicted in the same respective line as said Sll1441, and preferably the activity is increased non-targeted.

Accordingly, in one embodiment, the molecule which activity is to be increased in the process of the invention is the gene product with an activity as a “fatty acid desaturase”, preferably it is the molecule of section (a) or (b) of this paragraph.

In particular, it was observed that in plants, especially in Arabidopsis thaliana, increasing or generating the activity of a gene product non-targeted with the activity of a “fatty acid desaturase”, preferably being encoded by a gene comprising the nucleic acid sequence SEQ ID NO. 53608, preferably the coding region thereof, conferred the production of or the increase in glutamine compared with the wild type control.

The nucleic acid sequence of Sll1443 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of CTP synthetase.

Accordingly, in one embodiment, the process of the present invention for producing glutamate in a non-human organism, like a microorganism or a plant or a part thereof, comprises increasing or generating the activity of a gene product with the activity of a gene product conferring the activity of “CTP synthetase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll1443, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll1443, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll1443, or a functional equivalent or a homolog thereof as depicted in column 8 of Table II, application no. 2, preferably a homolog or functional equivalent as depicted in column 8 of Table II B, application no. 2, and being depicted in the same respective line as said Sll1443, and preferably the activity is increased mitochondrial.

Accordingly, in one embodiment, the molecule which activity is to be increased in the process of the invention is the gene product with an activity as a “CTP synthetase”, preferably it is the molecule of section (a) or (b) of this paragraph.

In particular, it was observed that in plants, especially in Arabidopsis thaliana, increasing or generating the activity of a gene product mitochondrial with the activity of a “CTP synthetase”, preferably being encoded by a gene comprising the nucleic acid sequence SEQ ID NO. 53878, preferably the coding region thereof, conferred the production of or the increase in glutamate compared with the wild type control.

The nucleic acid sequence of Sll1450 from Synechocystis sp., e.g. as shown in the respective line in column 5 of Table I, application no. 2, has been published in Synechocystis Nakamura et al., Nucleic Acids Res. 28(1), 72 (2000). And the activity of the gene product thereof is the activity of nitrate/nitrite transport protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “nitrate/nitrite transport protein”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll1450, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll1450, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll1450, or a functional equivalent or a homolog thereof as depicted in column 8 of Table II, application no. 2, preferably a homolog or functional equivalent as depicted in column 8 of Table II B, application no. 2, and being depicted in the same respective line as said Sll1450, and preferably the activity is increased mitochondrial.

Accordingly, in one embodiment, the molecule which activity is to be increased in the process of the invention is the gene product with an activity as a “nitrate/nitrite transport protein”, preferably it is the molecule of section (a) or (b) of this paragraph.

In particular, it was observed that in plants, especially in Arabidopsis thaliana, increasing or generating the activity of a gene product mitochondrial with the activity of a “nitrate/nitrite transport protein”, preferably being encoded by a gene comprising the nucleic acid sequence SEQ ID NO. 54337, preferably the coding region thereof, conferred the production of or the increase in glutamate compared with the wild type control.

The nucleic acid sequence of Sll1522 from Synechocystis sp., e.g. as shown in the respective line in column 5 of Table I, application no. 2, has been published in Synechocystis Nakamura et al., Nucleic Acids Res. 28(1), 72 (2000). And the activity of the gene product thereof is the activity of CDP-diacylglycerol-glycerol-3-phosphate 3-phosphatidyltransferase.

Accordingly, in one embodiment, the process of the present invention for producing glutamate in a non-human organism, like a microorganism or a plant or a part thereof, comprises increasing or generating the activity of a gene product with the activity of a gene product conferring the activity of “CDP-diacylglycerol-glycerol-3-phosphate 3-phosphatidyltransferase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll1522, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll1522, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll1522, or a functional equivalent or a homolog thereof as depicted in column 8 of Table II, application no. 2, preferably a homolog or functional equivalent as depicted in column 8 of Table II B, application no. 2, and being depicted in the same respective line as said Sll1522, and preferably the activity is increased plastidic.

Accordingly, in one embodiment, the molecule which activity is to be increased in the process of the invention is the gene product with an activity as a “CDP-diacylglycerol-glycerol-3-phosphate 3-phosphatidyltransferase”, preferably it is the molecule of section (a) or (b) of this paragraph.

In particular, it was observed that in plants, especially in Arabidopsis thaliana, increasing or generating the activity of a gene product plastidic with the activity of a “CDP-diacylglycerol-glycerol-3-phosphate 3-phosphatidyltransferase”, preferably being encoded by a gene comprising the nucleic acid sequence SEQ ID NO. 54452, preferably the coding region thereof, conferred the production of or the increase in glutamate compared with the wild type control.

The nucleic acid sequence of Sll1545 from Synechocystis sp., e.g. as shown in the respective line in column 5 of Table I, application no. 2, has been published in Synechocystis Nakamura et al., Nucleic Acids Res. 28(1), 72 (2000). And the activity of the gene product thereof is the activity of glutathione S-transferase.

Accordingly, in one embodiment, the process of the present invention for producing arginine in a non-human organism, like a microorganism or a plant or a part thereof, comprises increasing or generating the activity of a gene product with the activity of a gene product conferring the activity of “glutathione S-transferase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll1545, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll1545, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll1545, or a functional equivalent or a homolog thereof as depicted in column 8 of Table II, application no. 2, preferably a homolog or functional equivalent as depicted in column 8 of Table II B, application no. 2, and being depicted in the same respective line as said Sll1545, and preferably the activity is increased non-targeted.

Accordingly, in one embodiment, the molecule which activity is to be increased in the process of the invention is the gene product with an activity as a “glutathione S-transferase”, preferably it is the molecule of section (a) or (b) of this paragraph.

In particular, it was observed that in plants, especially in Arabidopsis thaliana, increasing or generating the activity of a gene product non-targeted with the activity of a “glutathione S-transferase”, preferably being encoded by a gene comprising the nucleic acid sequence SEQ ID NO. 11423, preferably the coding region thereof, conferred the production of or the increase in arginine compared with the wild type control.

The nucleic acid sequence of Sll1545 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of glutathione S-transferase.

Accordingly, in one embodiment, the process of the present invention for producing glutamine in a non-human organism, like a microorganism or a plant or a part thereof, comprises increasing or generating the activity of a gene product with the activity of a gene product conferring the activity of “glutathione S-transferase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll1545, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll1545, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll1545, or a functional equivalent or a homolog thereof as depicted in column 8 of Table II, application no. 2, preferably a homolog or functional equivalent as depicted in column 8 of Table II B, application no. 2, and being depicted in the same respective line as said Sll1545, and preferably the activity is increased mitochondrial.

Accordingly, in one embodiment, the molecule which activity is to be increased in the process of the invention is the gene product with an activity as a “glutathione S-transferase”, preferably it is the molecule of section (a) or (b) of this paragraph.

In particular, it was observed that in plants, especially in Arabidopsis thaliana, increasing or generating the activity of a gene product mitochondrial with the activity of a “glutathione S-transferase”, preferably being encoded by a gene comprising the nucleic acid sequence SEQ ID NO. 11423, preferably the coding region thereof, conferred the production of or the increase in glutamine compared with the wild type control.

The nucleic acid sequence of Sll1545 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of glutathione S-transferase.

Accordingly, in one embodiment, the process of the present invention for producing proline in a non-human organism, like a microorganism or a plant or a part thereof, comprises increasing or generating the activity of a gene product with the activity of a gene product conferring the activity of “glutathione S-transferase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll1545, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll1545, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll1545, or a functional equivalent or a homolog thereof as depicted in column 8 of Table II, application no. 2, preferably a homolog or functional equivalent as depicted in column 8 of Table II B, application no. 2, and being depicted in the same respective line as said Sll1545, and preferably the activity is increased mitochondrial.

Accordingly, in one embodiment, the molecule which activity is to be increased in the process of the invention is the gene product with an activity as a “glutathione S-transferase”, preferably it is the molecule of section (a) or (b) of this paragraph.

In particular, it was observed that in plants, especially in Arabidopsis thaliana, increasing or generating the activity of a gene product mitochondrial with the activity of a “glutathione S-transferase”, preferably being encoded by a gene comprising the nucleic acid sequence SEQ ID NO. 11423, preferably the coding region thereof, conferred the production of or the increase in proline compared with the wild type control.

The nucleic acid sequence of Sll1546 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of exopolyphosphatase.

Accordingly, in one embodiment, the process of the present invention for producing arginine in a non-human organism, like a microorganism or a plant or a part thereof, comprises increasing or generating the activity of a gene product with the activity of a gene product conferring the activity of “exopolyphosphatase”, especially from Synechocystis sp. or its functional equivalent or its homolog, e.g. the increase of (a) a gene product of a gene comprising the nucleic acid molecule as shown in the respective line in column 5 of Table I, application no. 2, preferably the coding region thereof, or a homolog or a fragment thereof, and being depicted in the same respective line as said Sll1546, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll1546, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll1546, or a functional equivalent or a homolog thereof as depicted in column 8 of Table II, application no. 2, preferably a homolog or functional equivalent as depicted in column 8 of Table II B, application no. 2, and being depicted in the same respective line as said

Sll1546, and preferably the activity is increased non-targeted.

Accordingly, in one embodiment, the molecule which activity is to be increased in the process of the invention is the gene product with an activity as a “exopolyphosphatase”, preferably it is the molecule of section (a) or (b) of this paragraph.

In particular, it was observed that in plants, especially in Arabidopsis thaliana, increasing or generating the activity of a gene product non-targeted with the activity of a “exopolyphosphatase”, preferably being encoded by a gene comprising the nucleic acid sequence SEQ ID NO. 54804, preferably the coding region thereof, conferred the production of or the increase in arginine compared with the wild type control.

The nucleic acid sequence of Sll1676 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of 4-alpha-glucanotransferase.

Accordingly, in one embodiment, the process of the present invention for producing proline in a non-human organism, like a microorganism or a plant or a part thereof, comprises increasing or generating the activity of a gene product with the activity of a gene product conferring the activity of “4-alpha-glucanotransferase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll1676, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll1676, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll1676, or a functional equivalent or a homolog thereof as depicted in column 8 of Table II, application no. 2, preferably a homolog or functional equivalent as depicted in column 8 of Table II B, application no. 2, and being depicted in the same respective line as said Sll1676, and preferably the activity is increased non-targeted.

Accordingly, in one embodiment, the molecule which activity is to be increased in the process of the invention is the gene product with an activity as a “4-alpha-glucanotransferase”, preferably it is the molecule of section (a) or (b) of this paragraph.

In particular, it was observed that in plants, especially in Arabidopsis thaliana, increasing or generating the activity of a gene product non-targeted with the activity of a “4-alphaglucanotransferase”, preferably being encoded by a gene comprising the nucleic acid sequence SEQ ID NO. 54897, preferably the coding region thereof, conferred the production of or the increase in proline compared with the wild type control.

The nucleic acid sequence of Sll1682 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of alanine dehydrogenase.

Accordingly, in one embodiment, the process of the present invention for producing proline in a non-human organism, like a microorganism or a plant or a part thereof, comprises increasing or generating the activity of a gene product with the activity of a gene product conferring the activity of “alanine dehydrogenase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll1682, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll1682, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll1682, or a functional equivalent or a homolog thereof as depicted in column 8 of Table II, application no. 2, preferably a homolog or functional equivalent as depicted in column 8 of Table II B, application no. 2, and being depicted in the same respective line as said Sll1682, and preferably the activity is increased plastidic.

Accordingly, in one embodiment, the molecule which activity is to be increased in the process of the invention is the gene product with an activity as a “alanine dehydrogenase”, preferably it is the molecule of section (a) or (b) of this paragraph.

In particular, it was observed that in plants, especially in Arabidopsis thaliana, increasing or generating the activity of a gene product plastidic with the activity of a “alanine dehydrogenase”, preferably being encoded by a gene comprising the nucleic acid sequence SEQ ID NO. 55063, preferably the coding region thereof, conferred the production of or the increase in proline compared with the wild type control.

The nucleic acid sequence of Sll1761 from Synechocystis sp., e.g. as shown in the respective line in column 5 of Table I, application no. 2, has been published in S Nakamura et al., Nucleic Acids Res. 28(1), 72 (2000). And the activity of the gene product thereof is the activity of Sll1761-protein.

Accordingly, in one embodiment, the process of the present invention for producing arginine in a non-human organism, like a microorganism or a plant or a part thereof, comprises increasing or generating the activity of a gene product with the activity of a gene product conferring the activity of “sll1761-protein”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll1761, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll1761, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll1761, or a functional equivalent or a homolog thereof as depicted in column 8 of Table II, application no. 2, preferably a homolog or functional equivalent as depicted in column 8 of Table II B, application no. 2, and being depicted in the same respective line as said Sll1761, and preferably the activity is increased non-targeted.

Accordingly, in one embodiment, the molecule which activity is to be increased in the process of the invention is the gene product with an activity as a “sll1761-protein”, preferably it is the molecule of section (a) or (b) of this paragraph.

In particular, it was observed that in plants, especially in Arabidopsis thaliana, increasing or generating the activity of a gene product non-targeted with the activity of a “sll1761-protein”, preferably being encoded by a gene comprising the nucleic acid sequence SEQ ID NO. 55379, preferably the coding region thereof, conferred the production of or the increase in arginine compared with the wild type control.

The nucleic acid sequence of Sll1761 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of sll1761-protein.

Accordingly, in one embodiment, the process of the present invention for producing proline in a non-human organism, like a microorganism or a plant or a part thereof, comprises increasing or generating the activity of a gene product with the activity of a gene product conferring the activity of “sll1761-protein”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll1761, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll1761, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll1761, or a functional equivalent or a homolog thereof as depicted in column 8 of Table II, application no. 2, preferably a homolog or functional equivalent as depicted in column 8 of Table II B, application no. 2, and being depicted in the same respective line as said Sll1761, and preferably the activity is increased non-targeted.

Accordingly, in one embodiment, the molecule which activity is to be increased in the process of the invention is the gene product with an activity as a “sll1761-protein”, preferably it is the molecule of section (a) or (b) of this paragraph.

In particular, it was observed that in plants, especially in Arabidopsis thaliana, increasing or generating the activity of a gene product non-targeted with the activity of a “sll1761-protein”, preferably being encoded by a gene comprising the nucleic acid sequence SEQ ID NO. 55379, preferably the coding region thereof, conferred the production of or the increase in proline compared with the wild type control.

The nucleic acid sequence of Sll1917 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of coproporphyrinogen III oxidase.

Accordingly, in one embodiment, the process of the present invention for producing glutamate in a non-human organism, like a microorganism or a plant or a part thereof, comprises increasing or generating the activity of a gene product with the activity of a gene product conferring the activity of “coproporphyrinogen III oxidase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll1917, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll1917, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll1917, or a functional equivalent or a homolog thereof as depicted in column 8 of Table II, application no. 2, preferably a homolog or functional equivalent as depicted in column 8 of Table II B, application no. 2, and being depicted in the same respective line as said Sll1917, and preferably the activity is increased mitochondrial.

Accordingly, in one embodiment, the molecule which activity is to be increased in the process of the invention is the gene product with an activity as a “coproporphyrinogen III oxidase”, preferably it is the molecule of section (a) or (b) of this paragraph.

In particular, it was observed that in plants, especially in Arabidopsis thaliana, increasing or generating the activity of a gene product mitochondrial with the activity of a “coproporphyrinogen III oxidase”, preferably being encoded by a gene comprising the nucleic acid sequence SEQ ID NO. 11471, preferably the coding region thereof, conferred the production of or the increase in glutamate compared with the wild type control.

The nucleic acid sequence of Sll1917 from Synechocystis sp., e.g. as shown in the respective line in column 5 of Table I, application no. 2, has been published in S Nakamura et al., Nucleic Acids Res. 28(1), 72 (2000). And the activity of the gene product thereof is the activity of coproporphyrinogen III oxidase.

Accordingly, in one embodiment, the process of the present invention for producing glutamine in a non-human organism, like a microorganism or a plant or a part thereof, comprises increasing or generating the activity of a gene product with the activity of a gene product conferring the activity of “coproporphyrinogen III oxidase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll1917, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll1917, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll1917, or a functional equivalent or a homolog thereof as depicted in column 8 of Table II, application no. 2, preferably a homolog or functional equivalent as depicted in column 8 of Table II B, application no. 2, and being depicted in the same respective line as said Sll1917, and preferably the activity is increased mitochondrial.

Accordingly, in one embodiment, the molecule which activity is to be increased in the process of the invention is the gene product with an activity as a “coproporphyrinogen III oxidase”, preferably it is the molecule of section (a) or (b) of this paragraph.

In particular, it was observed that in plants, especially in Arabidopsis thaliana, increasing or generating the activity of a gene product mitochondrial with the activity of a “coproporphyrinogen III oxidase”, preferably being encoded by a gene comprising the nucleic acid sequence SEQ ID NO. 11471, preferably the coding region thereof, conferred the production of or the increase in glutamine compared with the wild type control.

The nucleic acid sequence of Sll1917 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of coproporphyrinogen III oxidase.

Accordingly, in one embodiment, the process of the present invention for producing proline in a non-human organism, like a microorganism or a plant or a part thereof, comprises increasing or generating the activity of a gene product with the activity of a gene product conferring the activity of “coproporphyrinogen III oxidase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll1917, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll1917, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll1917, or a functional equivalent or a homolog thereof as depicted in column 8 of Table II, application no. 2, preferably a homolog or functional equivalent as depicted in column 8 of Table II B, application no. 2, and being depicted in the same respective line as said Sll1917, and preferably the activity is increased mitochondrial.

Accordingly, in one embodiment, the molecule which activity is to be increased in the process of the invention is the gene product with an activity as a “coproporphyrinogen III oxidase”, preferably it is the molecule of section (a) or (b) of this paragraph.

In particular, it was observed that in plants, especially in Arabidopsis thaliana, increasing or generating the activity of a gene product mitochondrial with the activity of a “coproporphyrinogen III oxidase”, preferably being encoded by a gene comprising the nucleic acid sequence SEQ ID NO. 11471, preferably the coding region thereof, conferred the production of or the increase in proline compared with the wild type control.

The nucleic acid sequence of Sll1920 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of cationtransporting ATPase.

Accordingly, in one embodiment, the process of the present invention for producing glutamate in a non-human organism, like a microorganism or a plant or a part thereof, comprises increasing or generating the activity of a gene product with the activity of a gene product conferring the activity of “cation-transporting ATPase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll1920, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll1920, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll1920, or a functional equivalent or a homolog thereof as depicted in column 8 of Table II, application no. 2, preferably a homolog or functional equivalent as depicted in column 8 of Table II B, application no. 2, and being depicted in the same respective line as said Sll1920, and preferably the activity is increased mitochondrial.

Accordingly, in one embodiment, the molecule which activity is to be increased in the process of the invention is the gene product with an activity as a “cation-transporting ATPase”, preferably it is the molecule of section (a) or (b) of this paragraph.

In particular, it was observed that in plants, especially in Arabidopsis thaliana, increasing or generating the activity of a gene product mitochondrial with the activity of a “cation-transporting ATPase”, preferably being encoded by a gene comprising the nucleic acid sequence SEQ ID NO. 55385, preferably the coding region thereof, conferred the production of or the increase in glutamate compared with the wild type control.

The nucleic acid sequence of Sll1920 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of cationtransporting ATPase.

Accordingly, in one embodiment, the process of the present invention for producing glutamine in a non-human organism, like a microorganism or a plant or a part thereof, comprises increasing or generating the activity of a gene product with the activity of a gene product conferring the activity of “cation-transporting ATPase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Sll1920, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Sll1920, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Sll1920, or a functional equivalent or a homolog thereof as depicted in column 8 of Table II, application no. 2, preferably a homolog or functional equivalent as depicted in column 8 of Table II B, application no. 2, and being depicted in the same respective line as said Sll1920, and preferably the activity is increased mitochondrial.

Accordingly, in one embodiment, the molecule which activity is to be increased in the process of the invention is the gene product with an activity as a “cation-transporting ATPase”, preferably it is the molecule of section (a) or (b) of this paragraph.

In particular, it was observed that in plants, especially in Arabidopsis thaliana, increasing or generating the activity of a gene product mitochondrial with the activity of a “cation-transporting ATPase”, preferably being encoded by a gene comprising the nucleic acid sequence SEQ ID NO. 55385, preferably the coding region thereof, conferred the production of or the increase in glutamine compared with the wild type control.

The nucleic acid sequence of Slr0237 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of glycosidase.

Accordingly, in one embodiment, the process of the present invention for producing arginine in a non-human organism, like a microorganism or a plant or a part thereof, comprises increasing or generating the activity of a gene product with the activity of a gene product conferring the activity of “glycosidase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr0237, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr0237, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr0237, or a functional equivalent or a homolog thereof as depicted in column 8 of Table II, application no. 2, preferably a homolog or functional equivalent as depicted in column 8 of Table II B, application no. 2, and being depicted in the same respective line as said Slr0237, and preferably the activity is increased non-targeted.

Accordingly, in one embodiment, the molecule which activity is to be increased in the process of the invention is the gene product with an activity as a “glycosidase”, preferably it is the molecule of section (a) or (b) of this paragraph.

In particular, it was observed that in plants, especially in Arabidopsis thaliana, increasing or generating the activity of a gene product non-targeted with the activity of a “glycosidase”, preferably being encoded by a gene comprising the nucleic acid sequence SEQ ID NO. 55771, preferably the coding region thereof, conferred the production of or the increase in arginine compared with the wild type control.

The nucleic acid sequence of Slr0237 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of glycosidase.

Accordingly, in one embodiment, the process of the present invention for producing glutamine in a non-human organism, like a microorganism or a plant or a part thereof, comprises increasing or generating the activity of a gene product with the activity of a gene product conferring the activity of “glycosidase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr0237, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr0237, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr0237, or a functional equivalent or a homolog thereof as depicted in column 8 of Table II, application no. 2, preferably a homolog or functional equivalent as depicted in column 8 of Table II B, application no. 2, and being depicted in the same respective line as said Slr0237, and preferably the activity is increased non-targeted.

Accordingly, in one embodiment, the molecule which activity is to be increased in the process of the invention is the gene product with an activity as a “glycosidase”, preferably it is the molecule of section (a) or (b) of this paragraph.

In particular, it was observed that in plants, especially in Arabidopsis thaliana, increasing or generating the activity of a gene product non-targeted with the activity of a “glycosidase”, preferably being encoded by a gene comprising the nucleic acid sequence SEQ ID NO. 55771, preferably the coding region thereof, conferred the production of or the increase in glutamine compared with the wild type control.

The nucleic acid sequence of Slr0477 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of phosphoribosylglycinamide formyltransferase.

Accordingly, in one embodiment, the process of the present invention for producing proline in a non-human organism, like a microorganism or a plant or a part thereof, comprises increasing or generating the activity of a gene product with the activity of a gene product conferring the activity of “phosphoribosylglycinamide formyltransferase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr0477, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr0477, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr0477, or a functional equivalent or a homolog thereof as depicted in column 8 of Table II, application no. 2, preferably a homolog or functional equivalent as depicted in column 8 of Table II B, application no. 2, and being depicted in the same respective line as said Slr0477, and preferably the activity is increased plastidic.

Accordingly, in one embodiment, the molecule which activity is to be increased in the process of the invention is the gene product with an activity as a “phosphoribosylglycinamide formyltransferase”, preferably it is the molecule of section (a) or (b) of this paragraph.

In particular, it was observed that in plants, especially in Arabidopsis thaliana, increasing or generating the activity of a gene product plastidic with the activity of a “phosphoribosylglycinamide formyltransferase”, preferably being encoded by a gene comprising the nucleic acid sequence SEQ ID NO. 55978, preferably the coding region thereof, conferred the production of or the increase in proline compared with the wild type control.

The nucleic acid sequence of Slr0597 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of bifunctional purine biosynthesis protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “bifunctional purine biosynthesis protein”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr0597, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr0597, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr0597, or a functional equivalent or a homolog thereof as depicted in column 8 of Table II, application no. 2, preferably a homolog or functional equivalent as depicted in column 8 of Table II B, application no. 2, and being depicted in the same respective line as said Slr0597, and preferably the activity is increased plastidic.

Accordingly, in one embodiment, the molecule which activity is to be increased in the process of the invention is the gene product with an activity as a “bifunctional purine biosynthesis protein”, preferably it is the molecule of section (a) or (b) of this paragraph.

In particular, it was observed that in plants, especially in Arabidopsis thaliana, increasing or generating the activity of a gene product plastidic with the activity of a “bifunctional purine biosynthesis protein”, preferably being encoded by a gene comprising the nucleic acid sequence SEQ ID NO. 56153, preferably the coding region thereof, conferred the production of or the increase in glutamate compared with the wild type control.

The nucleic acid sequence of Slr0600 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of slr0600-protein.

Accordingly, in one embodiment, the process of the present invention for producing proline in a non-human organism, like a microorganism or a plant or a part thereof, comprises increasing or generating the activity of a gene product with the activity of a gene product conferring the activity of “s1r0600-protein”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr0600, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr0600, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr0600, or a functional equivalent or a homolog thereof as depicted in column 8 of Table II, application no. 2, preferably a homolog or functional equivalent as depicted in column 8 of Table II B, application no. 2, and being depicted in the same respective line as said Slr0600, and preferably the activity is increased non-targeted.

Accordingly, in one embodiment, the molecule which activity is to be increased in the process of the invention is the gene product with an activity as a “slr0600-protein”, preferably it is the molecule of section (a) or (b) of this paragraph.

In particular, it was observed that in plants, especially in Arabidopsis thaliana, increasing or generating the activity of a gene product non-targeted with the activity of a “slr0600-protein”, preferably being encoded by a gene comprising the nucleic acid sequence SEQ ID NO. 56514, preferably the coding region thereof, conferred the production of or the increase in proline compared with the wild type control.

The nucleic acid sequence of Slr0661 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of pyrroline carboxylate reductase.

Accordingly, in one embodiment, the process of the present invention for producing proline in a non-human organism, like a microorganism or a plant or a part thereof, comprises increasing or generating the activity of a gene product with the activity of a gene product conferring the activity of “pyrroline carboxylate reductase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr0661, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr0661, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr0661, or a functional equivalent or a homolog thereof as depicted in column 8 of Table II, application no. 2, preferably a homolog or functional equivalent as depicted in column 8 of Table II B, application no. 2, and being depicted in the same respective line as said Slr0661, and preferably the activity is increased mitochondrial.

Accordingly, in one embodiment, the molecule which activity is to be increased in the process of the invention is the gene product with an activity as a “pyrroline carboxylate reductase”, preferably it is the molecule of section (a) or (b) of this paragraph.

In particular, it was observed that in plants, especially in Arabidopsis thaliana, increasing or generating the activity of a gene product mitochondrial with the activity of a “pyrroline carboxylate reductase”, preferably being encoded by a gene comprising the nucleic acid sequence SEQ ID NO. 56576, preferably the coding region thereof, conferred the production of or the increase in proline compared with the wild type control.

The nucleic acid sequence of Slr0710 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of Glu/Leu/Phe/Val dehydrogenase.

Accordingly, in one embodiment, the process of the present invention for producing arginine in a non-human organism, like a microorganism or a plant or a part thereof, comprises increasing or generating the activity of a gene product with the activity of a gene product conferring the activity of “Glu/Leu/PheNal dehydrogenase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr0710, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr0710, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr0710, or a functional equivalent or a homolog thereof as depicted in column 8 of Table II, application no. 2, preferably a homolog or functional equivalent as depicted in column 8 of Table II B, application no. 2, and being depicted in the same respective line as said Slr0710, and preferably the activity is increased mitochondrial.

Accordingly, in one embodiment, the molecule which activity is to be increased in the process of the invention is the gene product with an activity as a “Glu/Leu/PheNal dehydrogenase”, preferably it is the molecule of section (a) or (b) of this paragraph.

In particular, it was observed that in plants, especially in Arabidopsis thaliana, increasing or generating the activity of a gene product mitochondrial with the activity of a “Glu/Leu/Phe/Val dehydrogenase”, preferably being encoded by a gene comprising the nucleic acid sequence SEQ ID NO. 56894, preferably the coding region thereof, conferred the production of or the increase in arginine compared with the wild type control.

The nucleic acid sequence of Slr0739 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of geranylgeranyl pyrophosphate synthase.

Accordingly, in one embodiment, the process of the present invention for producing arginine in a non-human organism, like a microorganism or a plant or a part thereof, comprises increasing or generating the activity of a gene product with the activity of a gene product conferring the activity of “geranylgeranyl pyrophosphate synthase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr0739, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr0739, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr0739, or a functional equivalent or a homolog thereof as depicted in column 8 of Table II, application no. 2, preferably a homolog or functional equivalent as depicted in column 8 of Table II B, application no. 2, and being depicted in the same respective line as said Slr0739, and preferably the activity is increased plastidic.

Accordingly, in one embodiment, the molecule which activity is to be increased in the process of the invention is the gene product with an activity as a “geranylgeranyl pyrophosphate synthase”, preferably it is the molecule of section (a) or (b) of this paragraph.

In particular, it was observed that in plants, especially in Arabidopsis thaliana, increasing or generating the activity of a gene product plastidic with the activity of a “geranylgeranyl pyrophosphate synthase”, preferably being encoded by a gene comprising the nucleic acid sequence SEQ ID NO. 57235, preferably the coding region thereof, conferred the production of or the increase in arginine compared with the wild type control.

The nucleic acid sequence of Slr0739 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of geranylgeranyl pyrophosphate synthase.

Accordingly, in one embodiment, the process of the present invention for producing glutamine in a non-human organism, like a microorganism or a plant or a part thereof, comprises increasing or generating the activity of a gene product with the activity of a gene product conferring the activity of “geranylgeranyl pyrophosphate synthase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr0739, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr0739, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr0739, or a functional equivalent or a homolog thereof as depicted in column 8 of Table II, application no. 2, preferably a homolog or functional equivalent as depicted in column 8 of Table II B, application no. 2, and being depicted in the same respective line as said Slr0739, and preferably the activity is increased plastidic.

Accordingly, in one embodiment, the molecule which activity is to be increased in the process of the invention is the gene product with an activity as a “geranylgeranyl pyrophosphate synthase”, preferably it is the molecule of section (a) or (b) of this paragraph.

In particular, it was observed that in plants, especially in Arabidopsis thaliana, increasing or generating the activity of a gene product plastidic with the activity of a “geranylgeranyl pyrophosphate synthase”, preferably being encoded by a gene comprising the nucleic acid sequence SEQ ID NO. 57235, preferably the coding region thereof, conferred the production of or the increase in glutamine compared with the wild type control.

The nucleic acid sequence of Slr0739 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of geranylgeranyl pyrophosphate synthase.

Accordingly, in one embodiment, the process of the present invention for producing proline in a non-human organism, like a microorganism or a plant or a part thereof, comprises increasing or generating the activity of a gene product with the activity of a gene product conferring the activity of “geranylgeranyl pyrophosphate synthase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr0739, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr0739, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr0739, or a functional equivalent or a homolog thereof as depicted in column 8 of Table II, application no. 2, preferably a homolog or functional equivalent as depicted in column 8 of Table II B, application no. 2, and being depicted in the same respective line as said Slr0739, and preferably the activity is increased plastidic.

Accordingly, in one embodiment, the molecule which activity is to be increased in the process of the invention is the gene product with an activity as a “geranylgeranyl pyrophosphate synthase”, preferably it is the molecule of section (a) or (b) of this paragraph.

In particular, it was observed that in plants, especially in Arabidopsis thaliana, increasing or generating the activity of a gene product plastidic with the activity of a “geranylgeranyl pyrophosphate synthase”, preferably being encoded by a gene comprising the nucleic acid sequence SEQ ID NO. 57235, preferably the coding region thereof, conferred the production of or the increase in proline compared with the wild type control.

The nucleic acid sequence of Slr0756 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of circadian clock protein.

Accordingly, in one embodiment, the process of the present invention for producing arginine in a non-human organism, like a microorganism or a plant or a part thereof, comprises increasing or generating the activity of a gene product with the activity of a gene product conferring the activity of “circadian clock protein”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr0756, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr0756, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr0756, or a functional equivalent or a homolog thereof as depicted in column 8 of Table II, application no. 2, preferably a homolog or functional equivalent as depicted in column 8 of Table II B, application no. 2, and being depicted in the same respective line as said Slr0756, and preferably the activity is increased non-targeted.

Accordingly, in one embodiment, the molecule which activity is to be increased in the process of the invention is the gene product with an activity as a “circadian clock protein”, preferably it is the molecule of section (a) or (b) of this paragraph.

In particular, it was observed that in plants, especially in Arabidopsis thaliana, increasing or generating the activity of a gene product non-targeted with the activity of a “circadian clock protein”, preferably being encoded by a gene comprising the nucleic acid sequence SEQ ID NO. 57663, preferably the coding region thereof, conferred the production of or the increase in arginine compared with the wild type control.

The nucleic acid sequence of Slr0756 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of circadian clock protein.

Accordingly, in one embodiment, the process of the present invention for producing proline in a non-human organism, like a microorganism or a plant or a part thereof, comprises increasing or generating the activity of a gene product with the activity of a gene product conferring the activity of “circadian clock protein”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr0756, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr0756, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr0756, or a functional equivalent or a homolog thereof as depicted in column 8 of Table II, application no. 2, preferably a homolog or functional equivalent as depicted in column 8 of Table II B, application no. 2, and being depicted in the same respective line as said Slr0756, and preferably the activity is increased non-targeted.

Accordingly, in one embodiment, the molecule which activity is to be increased in the process of the invention is the gene product with an activity as a “circadian clock protein”, preferably it is the molecule of section (a) or (b) of this paragraph.

In particular, it was observed that in plants, especially in Arabidopsis thaliana, increasing or generating the activity of a gene product non-targeted with the activity of a “circadian clock protein”, preferably being encoded by a gene comprising the nucleic acid sequence SEQ ID NO. 57663, preferably the coding region thereof, conferred the production of or the increase in proline compared with the wild type control.

The nucleic acid sequence of Slr0782 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of amine oxidase.

Accordingly, in one embodiment, the process of the present invention for producing proline in a non-human organism, like a microorganism or a plant or a part thereof, comprises increasing or generating the activity of a gene product with the activity of a gene product conferring the activity of “amine oxidase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr0782, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr0782, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr0782, or a functional equivalent or a homolog thereof as depicted in column 8 of Table II, application no. 2, preferably a homolog or functional equivalent as depicted in column 8 of Table II B, application no. 2, and being depicted in the same respective line as said Slr0782, and preferably the activity is increased mitochondrial.

Accordingly, in one embodiment, the molecule which activity is to be increased in the process of the invention is the gene product with an activity as a “amine oxidase”, preferably it is the molecule of section (a) or (b) of this paragraph.

In particular, it was observed that in plants, especially in Arabidopsis thaliana, increasing or generating the activity of a gene product mitochondrial with the activity of a “amine oxidase”, preferably being encoded by a gene comprising the nucleic acid sequence SEQ ID NO. 57679, preferably the coding region thereof, conferred the production of or the increase in proline compared with the wild type control.

The nucleic acid sequence of Slr1096 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of dihydrolipoamide dehydrogenase.

Accordingly, in one embodiment, the process of the present invention for producing glutamate in a non-human organism, like a microorganism or a plant or a part thereof, comprises increasing or generating the activity of a gene product with the activity of a gene product conferring the activity of “dihydrolipoamide dehydrogenase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr1096, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr1096, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr1096, or a functional equivalent or a homolog thereof as depicted in column 8 of Table II, application no. 2, preferably a homolog or functional equivalent as depicted in column 8 of Table II B, application no. 2, and being depicted in the same respective line as said Slr1096, and preferably the activity is increased non-targeted.

Accordingly, in one embodiment, the molecule which activity is to be increased in the process of the invention is the gene product with an activity as a “dihydrolipoamide dehydrogenase”, preferably it is the molecule of section (a) or (b) of this paragraph.

In particular, it was observed that in plants, especially in Arabidopsis thaliana, increasing or generating the activity of a gene product non-targeted with the activity of a “dihydrolipoamide dehydrogenase”, preferably being encoded by a gene comprising the nucleic acid sequence SEQ ID NO. 57734, preferably the coding region thereof, conferred the production of or the increase in glutamate compared with the wild type control.

The nucleic acid sequence of Slr1269 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of gamma-glutamyltranspeptidase.

Accordingly, in one embodiment, the process of the present invention for producing arginine in a non-human organism, like a microorganism or a plant or a part thereof, comprises increasing or generating the activity of a gene product with the activity of a gene product conferring the activity of “gamma-glutamyltranspeptidase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr1269, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr1269, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr1269, or a functional equivalent or a homolog thereof as depicted in column 8 of Table II, application no. 2, preferably a homolog or functional equivalent as depicted in column 8 of Table II B, application no. 2, and being depicted in the same respective line as said Slr1269, and preferably the activity is increased non-targeted.

Accordingly, in one embodiment, the molecule which activity is to be increased in the process of the invention is the gene product with an activity as a “gamma-glutamyltranspeptidase”, preferably it is the molecule of section (a) or (b) of this paragraph.

In particular, it was observed that in plants, especially in Arabidopsis thaliana, increasing or generating the activity of a gene product non-targeted with the activity of a “gammaglutamyltranspeptidase”, preferably being encoded by a gene comprising the nucleic acid sequence SEQ ID NO. 58058, preferably the coding region thereof, conferred the production of or the increase in arginine compared with the wild type control.

The nucleic acid sequence of Slr1269 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of gamma-glutamyltranspeptidase.

Accordingly, in one embodiment, the process of the present invention for producing glutamate in a non-human organism, like a microorganism or a plant or a part thereof, comprises increasing or generating the activity of a gene product with the activity of a gene product conferring the activity of “gamma-glutamyltranspeptidase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr1269, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr1269, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr1269, or a functional equivalent or a homolog thereof as depicted in column 8 of Table II, application no. 2, preferably a homolog or functional equivalent as depicted in column 8 of Table II B, application no. 2, and being depicted in the same respective line as said Slr1269, and preferably the activity is increased non-targeted.

Accordingly, in one embodiment, the molecule which activity is to be increased in the process of the invention is the gene product with an activity as a “gamma-glutamyltranspeptidase”, preferably it is the molecule of section (a) or (b) of this paragraph.

In particular, it was observed that in plants, especially in Arabidopsis thaliana, increasing or generating the activity of a gene product non-targeted with the activity of a “gammaglutamyltranspeptidase”, preferably being encoded by a gene comprising the nucleic acid sequence SEQ ID NO. 58058, preferably the coding region thereof, conferred the production of or the increase in glutamate compared with the wild type control.

The nucleic acid sequence of Slr1312 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of arginine decarboxylase.

Accordingly, in one embodiment, the process of the present invention for producing arginine in a non-human organism, like a microorganism or a plant or a part thereof, comprises increasing or generating the activity of a gene product with the activity of a gene product conferring the activity of “arginine decarboxylase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr1312, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr1312, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr1312, or a functional equivalent or a homolog thereof as depicted in column 8 of Table II, application no. 2, preferably a homolog or functional equivalent as depicted in column 8 of Table II B, application no. 2, and being depicted in the same respective line as said Slr1312, and preferably the activity is increased non-targeted.

Accordingly, in one embodiment, the molecule which activity is to be increased in the process of the invention is the gene product with an activity as a “arginine decarboxylase”, preferably it is the molecule of section (a) or (b) of this paragraph.

In particular, it was observed that in plants, especially in Arabidopsis thaliana, increasing or generating the activity of a gene product non-targeted with the activity of a “arginine decarboxylase”, preferably being encoded by a gene comprising the nucleic acid sequence SEQ ID NO. 58324, preferably the coding region thereof, conferred the production of or the increase in arginine compared with the wild type control.

The nucleic acid sequence of Slr1369 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of CDP-diglyceride synthetase.

Accordingly, in one embodiment, the process of the present invention for producing glutamate in a non-human organism, like a microorganism or a plant or a part thereof, comprises increasing or generating the activity of a gene product with the activity of a gene product conferring the activity of “CDP-diglyceride synthetase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr1369, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr1369, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr1369, or a functional equivalent or a homolog thereof as depicted in column 8 of Table II, application no. 2, preferably a homolog or functional equivalent as depicted in column 8 of Table II B, application no. 2, and being depicted in the same respective line as said Slr1369, and preferably the activity is increased mitochondrial.

Accordingly, in one embodiment, the molecule which activity is to be increased in the process of the invention is the gene product with an activity as a “CDP-diglyceride synthetase”, preferably it is the molecule of section (a) or (b) of this paragraph.

In particular, it was observed that in plants, especially in Arabidopsis thaliana, increasing or generating the activity of a gene product mitochondrial with the activity of a “CDP-diglyceride synthetase”, preferably being encoded by a gene comprising the nucleic acid sequence SEQ ID NO. 58472, preferably the coding region thereof, conferred the production of or the increase in glutamate compared with the wild type control.

The nucleic acid sequence of Slr1420 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of carbohydrate kinase.

Accordingly, in one embodiment, the process of the present invention for producing glutamate in a non-human organism, like a microorganism or a plant or a part thereof, comprises increasing or generating the activity of a gene product with the activity of a gene product conferring the activity of “carbohydrate kinase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr1420, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr1420, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr1420, or a functional equivalent or a homolog thereof as depicted in column 8 of Table II, application no. 2, preferably a homolog or functional equivalent as depicted in column 8 of Table II B, application no. 2, and being depicted in the same respective line as said Slr1420, and preferably the activity is increased non-targeted.

Accordingly, in one embodiment, the molecule which activity is to be increased in the process of the invention is the gene product with an activity as a “carbohydrate kinase”, preferably it is the molecule of section (a) or (b) of this paragraph.

In particular, it was observed that in plants, especially in Arabidopsis thaliana, increasing or generating the activity of a gene product non-targeted with the activity of a “carbohydrate kinase”, preferably being encoded by a gene comprising the nucleic acid sequence SEQ ID NO. 58590, preferably the coding region thereof, conferred the production of or the increase in glutamate compared with the wild type control.

The nucleic acid sequence of Slr1492 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of iron(III) dicitrate-binding protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “iron(III) dicitrate-binding protein”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr1492, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr1492, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr1492, or a functional equivalent or a homolog thereof as depicted in column 8 of Table II, application no. 2, preferably a homolog or functional equivalent as depicted in column 8 of Table II B, application no. 2, and being depicted in the same respective line as said Slr1492, and preferably the activity is increased mitochondrial.

Accordingly, in one embodiment, the molecule which activity is to be increased in the process of the invention is the gene product with an activity as a “iron(III) dicitrate-binding protein”, preferably it is the molecule of section (a) or (b) of this paragraph.

In particular, it was observed that in plants, especially in Arabidopsis thaliana, increasing or generating the activity of a gene product mitochondrial with the activity of a “iron(III) dicitratebinding protein”, preferably being encoded by a gene comprising the nucleic acid sequence SEQ ID NO. 58668, preferably the coding region thereof, conferred the production of or the increase in glutamine compared with the wild type control.

The nucleic acid sequence of Slr1655 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of Photosystem I reaction center subunit XI.

Accordingly, in one embodiment, the process of the present invention for producing proline in a non-human organism, like a microorganism or a plant or a part thereof, comprises increasing or generating the activity of a gene product with the activity of a gene product conferring the activity of “Photosystem I reaction center subunit XI”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr1655, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr1655, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr1655, or a functional equivalent or a homolog thereof as depicted in column 8 of Table II, application no. 2, preferably a homolog or functional equivalent as depicted in column 8 of Table II B, application no. 2, and being depicted in the same respective line as said Slr1655, and preferably the activity is increased mitochondrial.

Accordingly, in one embodiment, the molecule which activity is to be increased in the process of the invention is the gene product with an activity as a “Photosystem I reaction center subunit XI”, preferably it is the molecule of section (a) or (b) of this paragraph.

In particular, it was observed that in plants, especially in Arabidopsis thaliana, increasing or generating the activity of a gene product mitochondrial with the activity of a “Photosystem I reaction center subunit XI”, preferably being encoded by a gene comprising the nucleic acid sequence SEQ ID NO. 12070, preferably the coding region thereof, conferred the production of or the increase in proline compared with the wild type control.

The nucleic acid sequence of Slr1739 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of photosystem II protein.

Accordingly, in one embodiment, the process of the present invention for producing arginine in a non-human organism, like a microorganism or a plant or a part thereof, comprises increasing or generating the activity of a gene product with the activity of a gene product conferring the activity of “photosystem II protein”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr1739, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr1739, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr1739, or a functional equivalent or a homolog thereof as depicted in column 8 of Table II, application no. 2, preferably a homolog or functional equivalent as depicted in column 8 of Table II B, application no. 2, and being depicted in the same respective line as said Slr1739, and preferably the activity is increased plastidic.

Accordingly, in one embodiment, the molecule which activity is to be increased in the process of the invention is the gene product with an activity as a “photosystem II protein”, preferably it is the molecule of section (a) or (b) of this paragraph.

In particular, it was observed that in plants, especially in Arabidopsis thaliana, increasing or generating the activity of a gene product plastidic with the activity of a “photosystem II protein”, preferably being encoded by a gene comprising the nucleic acid sequence SEQ ID NO. 58731, preferably the coding region thereof, conferred the production of or the increase in arginine compared with the wild type control.

The nucleic acid sequence of Slr1739 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of photosystem II protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “photosystem II protein”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr1739, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr1739, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr1739, or a functional equivalent or a homolog thereof as depicted in column 8 of Table II, application no. 2, preferably a homolog or functional equivalent as depicted in column 8 of Table II B, application no. 2, and being depicted in the same respective line as said Slr1739, and preferably the activity is increased plastidic.

Accordingly, in one embodiment, the molecule which activity is to be increased in the process of the invention is the gene product with an activity as a “photosystem II protein”, preferably it is the molecule of section (a) or (b) of this paragraph.

In particular, it was observed that in plants, especially in Arabidopsis thaliana, increasing or generating the activity of a gene product plastidic with the activity of a “photosystem II protein”, preferably being encoded by a gene comprising the nucleic acid sequence SEQ ID NO. 58731, preferably the coding region thereof, conferred the production of or the increase in glutamine compared with the wild type control.

The nucleic acid sequence of Slr1742 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of glutamine amidotransferase.

Accordingly, in one embodiment, the process of the present invention for producing proline in a non-human organism, like a microorganism or a plant or a part thereof, comprises increasing or generating the activity of a gene product with the activity of a gene product conferring the activity of “glutamine amidotransferase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr1742, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr1742, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr1742, or a functional equivalent or a homolog thereof as depicted in column 8 of Table II, application no. 2, preferably a homolog or functional equivalent as depicted in column 8 of Table II B, application no. 2, and being depicted in the same respective line as said Slr1742, and preferably the activity is increased mitochondrial.

Accordingly, in one embodiment, the molecule which activity is to be increased in the process of the invention is the gene product with an activity as a “glutamine amidotransferase”, preferably it is the molecule of section (a) or (b) of this paragraph.

In particular, it was observed that in plants, especially in Arabidopsis thaliana, increasing or generating the activity of a gene product mitochondrial with the activity of a “glutamine amidotransferase”, preferably being encoded by a gene comprising the nucleic acid sequence SEQ ID NO. 58751, preferably the coding region thereof, conferred the production of or the increase in proline compared with the wild type control.

The nucleic acid sequence of Slr1755 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of glycerol-3-phosphate dehydrogenase.

Accordingly, in one embodiment, the process of the present invention for producing glutamate in a non-human organism, like a microorganism or a plant or a part thereof, comprises increasing or generating the activity of a gene product with the activity of a gene product conferring the activity of “glycerol-3-phosphate dehydrogenase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr1755, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr1755, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr1755, or a functional equivalent or a homolog thereof as depicted in column 8 of Table II, application no. 2, preferably a homolog or functional equivalent as depicted in column 8 of Table II B, application no. 2, and being depicted in the same respective line as said Slr1755, and preferably the activity is increased mitochondrial.

Accordingly, in one embodiment, the molecule which activity is to be increased in the process of the invention is the gene product with an activity as a “glycerol-3-phosphate dehydrogenase”, preferably it is the molecule of section (a) or (b) of this paragraph.

In particular, it was observed that in plants, especially in Arabidopsis thaliana, increasing or generating the activity of a gene product mitochondrial with the activity of a “glycerol-3-phosphate dehydrogenase”, preferably being encoded by a gene comprising the nucleic acid sequence SEQ ID NO. 58823, preferably the coding region thereof, conferred the production of or the increase in glutamate compared with the wild type control.

The nucleic acid sequence of Slr1791 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of phosphoadenosine phosphosulfate reductase.

Accordingly, in one embodiment, the process of the present invention for producing proline in a non-human organism, like a microorganism or a plant or a part thereof, comprises increasing or generating the activity of a gene product with the activity of a gene product conferring the activity of “phosphoadenosine phosphosulfate reductase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr1791, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr1791, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr1791, or a functional equivalent or a homolog thereof as depicted in column 8 of Table II, application no. 2, preferably a homolog or functional equivalent as depicted in column 8 of Table II B, application no. 2, and being depicted in the same respective line as said Slr1791, and preferably the activity is increased plastidic.

Accordingly, in one embodiment, the molecule which activity is to be increased in the process of the invention is the gene product with an activity as a “phosphoadenosine phosphosulfate reductase”, preferably it is the molecule of section (a) or (b) of this paragraph.

In particular, it was observed that in plants, especially in Arabidopsis thaliana, increasing or generating the activity of a gene product plastidic with the activity of a “phosphoadenosine phosphosulfate reductase”, preferably being encoded by a gene comprising the nucleic acid sequence SEQ ID NO. 12140, preferably the coding region thereof, conferred the production of or the increase in proline compared with the wild type control.

The nucleic acid sequence of Slr1882 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of riboflavin biosynthesis protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “riboflavin biosynthesis protein”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr1882, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr1882, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr1882, or a functional equivalent or a homolog thereof as depicted in column 8 of Table II, application no. 2, preferably a homolog or functional equivalent as depicted in column 8 of Table II B, application no. 2, and being depicted in the same respective line as said Slr1882, and preferably the activity is increased non-targeted.

Accordingly, in one embodiment, the molecule which activity is to be increased in the process of the invention is the gene product with an activity as a “riboflavin biosynthesis protein”, preferably it is the molecule of section (a) or (b) of this paragraph.

In particular, it was observed that in plants, especially in Arabidopsis thaliana, increasing or generating the activity of a gene product non-targeted with the activity of a “riboflavin biosynthesis protein”, preferably being encoded by a gene comprising the nucleic acid sequence SEQ ID NO. 59041, preferably the coding region thereof, conferred the production of or the increase in glutamate compared with the wild type control.

The nucleic acid sequence of Slr2023 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of malonyl CoA-acyl carrier protein transacylase.

Accordingly, in one embodiment, the process of the present invention for producing glutamate in a non-human organism, like a microorganism or a plant or a part thereof, comprises increasing or generating the activity of a gene product with the activity of a gene product conferring the activity of “malonyl CoA-acyl carrier protein transacylase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr2023, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr2023, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr2023, or a functional equivalent or a homolog thereof as depicted in column 8 of Table II, application no. 2, preferably a homolog or functional equivalent as depicted in column 8 of Table II B, application no. 2, and being depicted in the same respective line as said Slr2023, and preferably the activity is increased plastidic.

Accordingly, in one embodiment, the molecule which activity is to be increased in the process of the invention is the gene product with an activity as a “malonyl CoA-acyl carrier protein transacylase”, preferably it is the molecule of section (a) or (b) of this paragraph.

In particular, it was observed that in plants, especially in Arabidopsis thaliana, increasing or generating the activity of a gene product plastidic with the activity of a “malonyl CoA-acyl carrier protein transacylase”, preferably being encoded by a gene comprising the nucleic acid sequence SEQ ID NO. 59165, preferably the coding region thereof, conferred the production of or the increase in glutamate compared with the wild type control.

The nucleic acid sequence of Slr2023 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of malonyl CoA-acyl carrier protein transacylase.

Accordingly, in one embodiment, the process of the present invention for producing glutamine in a non-human organism, like a microorganism or a plant or a part thereof, comprises increasing or generating the activity of a gene product with the activity of a gene product conferring the activity of “malonyl CoA-acyl carrier protein transacylase”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr2023, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr2023, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr2023, or a functional equivalent or a homolog thereof as depicted in column 8 of Table II, application no. 2, preferably a homolog or functional equivalent as depicted in column 8 of Table II B, application no. 2, and being depicted in the same respective line as said Slr2023, and preferably the activity is increased plastidic.

Accordingly, in one embodiment, the molecule which activity is to be increased in the process of the invention is the gene product with an activity as a “malonyl CoA-acyl carrier protein transacylase”, preferably it is the molecule of section (a) or (b) of this paragraph.

In particular, it was observed that in plants, especially in Arabidopsis thaliana, increasing or generating the activity of a gene product plastidic with the activity of a “malonyl CoA-acyl carrier protein transacylase”, preferably being encoded by a gene comprising the nucleic acid sequence SEQ ID NO. 59165, preferably the coding region thereof, conferred the production of or the increase in glutamine compared with the wild type control.

The nucleic acid sequence of Slr2124 from Synechocystis sp., e.g. as shown in the respective line in column 5 of Table I, application no. 2, has been published in Nakamura et al., Nucleic Acids Res. 28(1), 72 (2000). And the activity of the gene product thereof is the activity of shortchain alcohol dehydrogenase family.

Accordingly, in one embodiment, the process of the present invention for producing proline in a non-human organism, like a microorganism or a plant or a part thereof, comprises increasing or generating the activity of a gene product with the activity of a gene product conferring the activity of “short-chain alcohol dehydrogenase family”, especially from Synechocystis sp. or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Slr2124, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Slr2124, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Slr2124, or a functional equivalent or a homolog thereof as depicted in column 8 of Table II, application no. 2, preferably a homolog or functional equivalent as depicted in column 8 of Table II B, application no. 2, and being depicted in the same respective line as said Slr2124, and preferably the activity is increased plastidic.

Accordingly, in one embodiment, the molecule which activity is to be increased in the process of the invention is the gene product with an activity as a “short-chain alcohol dehydrogenase family”, preferably it is the molecule of section (a) or (b) of this paragraph.

In particular, it was observed that in plants, especially in Arabidopsis thaliana, increasing or generating the activity of a gene product plastidic with the activity of a “short-chain alcohol dehydrogenase family”, preferably being encoded by a gene comprising the nucleic acid sequence SEQ ID NO. 59370, preferably the coding region thereof, conferred the production of or the increase in proline compared with the wild type control.

The nucleic acid sequence of TTC0019 from Thermus thermophilus, e.g. as shown in the respective line in column 5 of Table I, application no. 2, has been published in Henne et al., Nat. Biotechnol. 22 (5), 547 (2004). And the activity of the gene product thereof is the activity of Sec-independent protein translocase subunit.

Accordingly, in one embodiment, the process of the present invention for producing glutamine in a non-human organism, like a microorganism or a plant or a part thereof, comprises increasing or generating the activity of a gene product with the activity of a gene product conferring the activity of “Sec-independent protein translocase subunit”, especially from Thermus thermophilus or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said TTC0019, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said TTC0019, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said TTC0019, or a functional equivalent or a homolog thereof as depicted in column 8 of Table II, application no. 2, preferably a homolog or functional equivalent as depicted in column 8 of Table II B, application no. 2, and being depicted in the same respective line as said TTC0019, and preferably the activity is increased non-targeted.

Accordingly, in one embodiment, the molecule which activity is to be increased in the process of the invention is the gene product with an activity as a “Sec-independent protein translocase subunit”, preferably it is the molecule of section (a) or (b) of this paragraph. In particular, it was observed that in plants, especially in Arabidopsis thaliana, increasing or generating the activity of a gene product non-targeted with the activity of a “Sec-independent protein translocase subunit”, preferably being encoded by a gene comprising the nucleic acid sequence SEQ ID NO. 12698, preferably the coding region thereof, conferred the production of or the increase in glutamine compared with the wild type control.

The nucleic acid sequence of TTC0019 from Thermus thermophilus, e.g. as shown in the respective line in column 5 of Table I, application no. 2, has been published in S Henne et al., Nat. Biotechnol. 22 (5), 547 (2004). And the activity of the gene product thereof is the activity of Sec-independent protein translocase subunit.

Accordingly, in one embodiment, the process of the present invention for producing proline in a non-human organism, like a microorganism or a plant or a part thereof, comprises increasing or generating the activity of a gene product with the activity of a gene product conferring the activity of “Sec-independent protein translocase subunit”, especially from Thermus thermophilus or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said TTC0019, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said TTC0019, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said TTC0019, or a functional equivalent or a homolog thereof as depicted in column 8 of Table II, application no. 2, preferably a homolog or functional equivalent as depicted in column 8 of Table II B, application no. 2, and being depicted in the same respective line as said TTC0019, and preferably the activity is increased non-targeted.

Accordingly, in one embodiment, the molecule which activity is to be increased in the process of the invention is the gene product with an activity as a “Sec-independent protein translocase subunit”, preferably it is the molecule of section (a) or (b) of this paragraph.

In particular, it was observed that in plants, especially in Arabidopsis thaliana, increasing or generating the activity of a gene product non-targeted with the activity of a “Sec-independent protein translocase subunit”, preferably being encoded by a gene comprising the nucleic acid sequence SEQ ID NO. 12698, preferably the coding region thereof, conferred the production of or the increase in proline compared with the wild type control.

The nucleic acid sequence of TTC0035 from Thermus thermophilus, e.g. as shown in the respective line in column 5 of Table I, application no. 2, has been published in Henne et al., Nat. Biotechnol. 22 (5), 547 (2004). And the activity of the gene product thereof is the activity of cell division protein.

Accordingly, in one embodiment, the process of the present invention for producing proline in a non-human organism, like a microorganism or a plant or a part thereof, comprises increasing or generating the activity of a gene product with the activity of a gene product conferring the activity of “cell division protein”, especially from Thermus thermophilus or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said TTC0035, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said TTC0035, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said TTC0035, or a functional equivalent or a homolog thereof as depicted in column 8 of Table II, application no. 2, preferably a homolog or functional equivalent as depicted in column 8 of Table II B, application no. 2, and being depicted in the same respective line as said TTC0035, and preferably the activity is increased non-targeted.

Accordingly, in one embodiment, the molecule which activity is to be increased in the process of the invention is the gene product with an activity as a “cell division protein”, preferably it is the molecule of section (a) or (b) of this paragraph.

In particular, it was observed that in plants, especially in Arabidopsis thaliana, increasing or generating the activity of a gene product non-targeted with the activity of a “cell division protein”, preferably being encoded by a gene comprising the nucleic acid sequence SEQ ID NO. 60301, preferably the coding region thereof, conferred the production of or the increase in proline compared with the wild type control.

The nucleic acid sequence of TTC0216 from Thermus thermophilus, e.g. as shown in the respective line in column 5 of Table I, application no. 2, has been published in Henne et al., Nat. Biotechnol. 22 (5), 547 (2004). And the activity of the gene product thereof is the activity of branched-chain amino acid ABC transporter permease protein.

Accordingly, in one embodiment, the process of the present invention for producing proline in a non-human organism, like a microorganism or a plant or a part thereof, comprises increasing or generating the activity of a gene product with the activity of a gene product conferring the activity of “branched-chain amino acid ABC transporter permease protein”, especially from Thermus thermophilus or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said TTC0216, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said TTC0216, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said TTC0216, or a functional equivalent or a homolog thereof as depicted in column 8 of Table II, application no. 2, preferably a homolog or functional equivalent as depicted in column 8 of Table II B, application no. 2, and being depicted in the same respective line as said TTC0216, and preferably the activity is increased non-targeted.

Accordingly, in one embodiment, the molecule which activity is to be increased in the process of the invention is the gene product with an activity as a “branched-chain amino acid ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “branched-chain amino acid ABC transporter permease protein”, preferably being encoded by a gene comprising the nucleic acid sequence SEQ ID NO. 60859, preferably the coding region thereof, conferred the production of or the increase in proline compared with the wild type control.

The nucleic acid sequence of TTC0337 from Thermus thermophilus, e.g. as shown in the respective line in column 5 of Table I, application no. 2, has been published in Henne et al., Nat. Biotechnol. 22 (5), 547 (2004). And the activity of the gene product thereof is the activity of amino acid ABC transporter permease protein.

Accordingly, in one embodiment, the process of the present invention for producing proline in a non-human organism, like a microorganism or a plant or a part thereof, comprises increasing or generating the activity of a gene product with the activity of a gene product conferring the activity of “amino acid ABC transporter permease protein”, especially from Thermus thermophilus or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said TTC0337, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said TTC0337, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said TTC0337, or a functional equivalent or a homolog thereof as depicted in column 8 of Table II, application no. 2, preferably a homolog or functional equivalent as depicted in column 8 of Table II B, application no. 2, and being depicted in the same respective line as said TTC0337, and preferably the activity is increased non-targeted.

Accordingly, in one embodiment, the molecule which activity is to be increased in the process of the invention is the gene product with an activity as a “amino acid ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “amino acid ABC transporter permease protein”, preferably being encoded by a gene comprising the nucleic acid sequence SEQ ID NO. 61070, preferably the coding region thereof, conferred the production of or the increase in proline compared with the wild type control.

The nucleic acid sequence of TTC0768 from Thermus thermophilus, e.g. as shown in the respective line in column 5 of Table I, application no. 2, has been published in Henne et al., Nat. Biotechnol. 22 (5), 547 (2004). And the activity of the gene product thereof is the activity of TTC0768-protein.

Accordingly, in one embodiment, the process of the present invention for producing proline in a non-human organism, like a microorganism or a plant or a part thereof, comprises increasing or generating the activity of a gene product with the activity of a gene product conferring the activity of “TTC0768-protein”, especially from Thermus thermophilus or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said TTC0768, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said TTC0768, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said TTC0768, or a functional equivalent or a homolog thereof as depicted in column 8 of Table II, application no. 2, preferably a homolog or functional equivalent as depicted in column 8 of Table II B, application no. 2, and being depicted in the same respective line as said TTC0768, and preferably the activity is increased non-targeted.

Accordingly, in one embodiment, the molecule which activity is to be increased in the process of the invention is the gene product with an activity as a “TTC0768-protein”, preferably it is the molecule of section (a) or (b) of this paragraph.

In particular, it was observed that in plants, especially in Arabidopsis thaliana, increasing or generating the activity of a gene product non-targeted with the activity of a “TTC0768-protein”, preferably being encoded by a gene comprising the nucleic acid sequence SEQ ID NO. 61532, preferably the coding region thereof, conferred the production of or the increase in proline compared with the wild type control.

The nucleic acid sequence of TTC0917 from Thermus thermophilus, e.g. as shown in the respective line in column 5 of Table I, application no. 2, has been published in Henne et al., Nat. Biotechnol. 22 (5), 547 (2004). And the activity of the gene product thereof is the activity of metal-dependent hydrolase.

Accordingly, in one embodiment, the process of the present invention for producing glutamine in a non-human organism, like a microorganism or a plant or a part thereof, comprises increasing or generating the activity of a gene product with the activity of a gene product conferring the activity of “metal-dependent hydrolase”, especially from Thermus thermophilus or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said TTC0917, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said TTC0917, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said TTC0917, or a functional equivalent or a homolog thereof as depicted in column 8 of Table II, application no. 2, preferably a homolog or functional equivalent as depicted in column 8 of Table II B, application no. 2, and being depicted in the same respective line as said TTC0917, and preferably the activity is increased non-targeted.

Accordingly, in one embodiment, the molecule which activity is to be increased in the process of the invention is the gene product with an activity as a “metal-dependent hydrolase”, preferably it is the molecule of section (a) or (b) of this paragraph.

In particular, it was observed that in plants, especially in Arabidopsis thaliana, increasing or generating the activity of a gene product non-targeted with the activity of a “metal-dependent hydrolase”, preferably being encoded by a gene comprising the nucleic acid sequence SEQ ID NO. 61553, preferably the coding region thereof, conferred the production of or the increase in glutamine compared with the wild type control.

The nucleic acid sequence of TTC1193 from Thermus thermophilus, e.g. as shown in the respective line in column 5 of Table I, application no. 2, has been published in Henne et al., Nat. Biotechnol. 22 (5), 547 (2004). And the activity of the gene product thereof is the activity of multiple antibiotic resistance protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “multiple antibiotic resistance protein”, especially from Thermus thermophilus or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said TTC1193, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said TTC1193, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said TTC1193, or a functional equivalent or a homolog thereof as depicted in column 8 of Table II, application no. 2, preferably a homolog or functional equivalent as depicted in column 8 of Table II B, application no. 2, and being depicted in the same respective line as said TTC1193, and preferably the activity is increased non-targeted.

Accordingly, in one embodiment, the molecule which activity is to be increased in the process of the invention is the gene product with an activity as a “multiple antibiotic resistance protein”, preferably it is the molecule of section (a) or (b) of this paragraph.

In particular, it was observed that in plants, especially in Arabidopsis thaliana, increasing or generating the activity of a gene product non-targeted with the activity of a “multiple antibiotic resistance protein”, preferably being encoded by a gene comprising the nucleic acid sequence SEQ ID NO. 61723, preferably the coding region thereof, conferred the production of or the increase in glutamate compared with the wild type control.

The nucleic acid sequence of TTC1386 from Thermus thermophilus, e.g. as shown in the respective line in column 5 of Table I, application no. 2, has been published in Henne et al., Nat. Biotechnol. 22 (5), 547 (2004). And the activity of the gene product thereof is the activity of TTC1386-protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “TTC1386-protein”, especially from Thermus thermophilus or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said TTC1386, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said TTC1386, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said TTC1386, or a functional equivalent or a homolog thereof as depicted in column 8 of Table II, application no. 2, preferably a homolog or functional equivalent as depicted in column 8 of Table II B, application no. 2, and being depicted in the same respective line as said TTC1386, and preferably the activity is increased non-targeted.

Accordingly, in one embodiment, the molecule which activity is to be increased in the process of the invention is the gene product with an activity as a “TTC1386-protein”, preferably it is the molecule of section (a) or (b) of this paragraph.

In particular, it was observed that in plants, especially in Arabidopsis thaliana, increasing or generating the activity of a gene product non-targeted with the activity of a “TTC1386-protein”, preferably being encoded by a gene comprising the nucleic acid sequence SEQ ID NO. 62079, preferably the coding region thereof, conferred the production of or the increase in glutamate compared with the wild type control.

The nucleic acid sequence of TTC1550 from Thermus thermophilus, e.g. as shown in the respective line in column 5 of Table I, application no. 2, has been published in Henne et al., Nat. Biotechnol. 22 (5), 547 (2004). And the activity of the gene product thereof is the activity of homocitrate synthase.

Accordingly, in one embodiment, the process of the present invention for producing glutamine in a non-human organism, like a microorganism or a plant or a part thereof, comprises increasing or generating the activity of a gene product with the activity of a gene product conferring the activity of “homocitrate synthase”, especially from Thermus thermophilus or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said TTC1550, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said TTC1550, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said TTC1550, or a functional equivalent or a homolog thereof as depicted in column 8 of Table II, application no. 2, preferably a homolog or functional equivalent as depicted in column 8 of Table II B, application no. 2, and being depicted in the same respective line as said TTC1550, and preferably the activity is increased non-targeted.

Accordingly, in one embodiment, the molecule which activity is to be increased in the process of the invention is the gene product with an activity as a “homocitrate synthase”, preferably it is the molecule of section (a) or (b) of this paragraph.

In particular, it was observed that in plants, especially in Arabidopsis thaliana, increasing or generating the activity of a gene product non-targeted with the activity of a “homocitrate synthase”, preferably being encoded by a gene comprising the nucleic acid sequence SEQ ID NO. 12974, preferably the coding region thereof, conferred the production of or the increase in glutamine compared with the wild type control.

The nucleic acid sequence of TTC1550 from Thermus thermophilus, e.g. as shown in the respective line in column 5 of Table I, application no. 2, has been published in Henne et al., Nat. Biotechnol. 22 (5), 547 (2004). And the activity of the gene product thereof is the activity of homocitrate synthase.

Accordingly, in one embodiment, the process of the present invention for producing proline in a non-human organism, like a microorganism or a plant or a part thereof, comprises increasing or generating the activity of a gene product with the activity of a gene product conferring the activity of “homocitrate synthase”, especially from Thermus thermophilus or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said TTC1550, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said TTC1550, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said TTC1550, or a functional equivalent or a homolog thereof as depicted in column 8 of Table II, application no. 2, preferably a homolog or functional equivalent as depicted in column 8 of Table II B, application no. 2, and being depicted in the same respective line as said TTC1550, and preferably the activity is increased non-targeted.

Accordingly, in one embodiment, the molecule which activity is to be increased in the process of the invention is the gene product with an activity as a “homocitrate synthase”, preferably it is the molecule of section (a) or (b) of this paragraph.

In particular, it was observed that in plants, especially in Arabidopsis thaliana, increasing or generating the activity of a gene product non-targeted with the activity of a “homocitrate synthase”, preferably being encoded by a gene comprising the nucleic acid sequence SEQ ID NO. 12974, preferably the coding region thereof, conferred the production of or the increase in proline compared with the wild type control.

The nucleic acid sequence of TTC1918 from Thermus thermophilus, e.g. as shown in the respective line in column 5 of Table I, application no. 2, has been published in Henne et al., Nat. Biotechnol. 22 (5), 547 (2004). And the activity of the gene product thereof is the activity of oxidoreductase subunit.

Accordingly, in one embodiment, the process of the present invention for producing glutamate in a non-human organism, like a microorganism or a plant or a part thereof, comprises increasing or generating the activity of a gene product with the activity of a gene product conferring the activity of “oxidoreductase subunit”, especially from Thermus thermophilus or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said TTC1918, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said TTC1918, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said TTC1918, or a functional equivalent or a homolog thereof as depicted in column 8 of Table II, application no. 2, preferably a homolog or functional equivalent as depicted in column 8 of Table II B, application no. 2, and being depicted in the same respective line as said TTC1918, and preferably the activity is increased non-targeted.

Accordingly, in one embodiment, the molecule which activity is to be increased in the process of the invention is the gene product with an activity as a “oxidoreductase subunit”, preferably it is the molecule of section (a) or (b) of this paragraph.

In particular, it was observed that in plants, especially in Arabidopsis thaliana, increasing or generating the activity of a gene product non-targeted with the activity of a “oxidoreductase subunit”, preferably being encoded by a gene comprising the nucleic acid sequence SEQ ID NO. 62160, preferably the coding region thereof, conferred the production of or the increase in glutamate compared with the wild type control.

The nucleic acid sequence of TTC1918 from Thermus thermophilus, e.g. as shown in the respective line in column 5 of Table I, application no. 2, has been published in Henne et al., Nat. Biotechnol. 22 (5), 547 (2004). And the activity of the gene product thereof is the activity of oxidoreductase subunit.

Accordingly, in one embodiment, the process of the present invention for producing glutamine in a non-human organism, like a microorganism or a plant or a part thereof, comprises increasing or generating the activity of a gene product with the activity of a gene product conferring the activity of “oxidoreductase subunit”, especially from Thermus thermophilus or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said TTC1918, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said TTC1918, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said TTC1918, or a functional equivalent or a homolog thereof as depicted in column 8 of Table II, application no. 2, preferably a homolog or functional equivalent as depicted in column 8 of Table II B, application no. 2, and being depicted in the same respective line as said TTC1918, and preferably the activity is increased non-targeted.

Accordingly, in one embodiment, the molecule which activity is to be increased in the process of the invention is the gene product with an activity as a “oxidoreductase subunit”, preferably it is the molecule of section (a) or (b) of this paragraph.

In particular, it was observed that in plants, especially in Arabidopsis thaliana, increasing or generating the activity of a gene product non-targeted with the activity of a “oxidoreductase subunit”, preferably being encoded by a gene comprising the nucleic acid sequence SEQ ID NO. 62160, preferably the coding region thereof, conferred the production of or the increase in glutamine compared with the wild type control.

The nucleic acid sequence of XM_{—}473199 from Oryza sativa, e.g. as shown in the respective line in column 5 of Table I, application no. 2 is unpublished. And the activity of the gene product thereof is the activity of XM_{—}473199-protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “XM_{—}473199-protein”, especially from Oryza sativa or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said XM_{—}473199, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said XM_{—}473199, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said XM_{—}473199, or a functional equivalent or a homolog thereof as depicted in column 8 of Table II, application no. 2, preferably a homolog or functional equivalent as depicted in column 8 of Table II B, application no. 2, and being depicted in the same respective line as said XM_{—}473199, and preferably the activity is increased non-targeted.

Accordingly, in one embodiment, the molecule which activity is to be increased in the process of the invention is the gene product with an activity as a “XM_{—}473199-protein”, preferably it is the molecule of section (a) or (b) of this paragraph.

In particular, it was observed that in plants, especially in Arabidopsis thaliana, increasing or generating the activity of a gene product non-targeted with the activity of a “XM_{—}473199-protein”, preferably being encoded by a gene comprising the nucleic acid sequence SEQ ID NO. 62244, preferably the coding region thereof, conferred the production of or the increase in glutamate compared with the wild type control.

The nucleic acid sequence of XM_{—}473199 from Oryza sativa, e.g. as shown in the respective line in column 5 of Table I, application no. 2 is unpublished. And the activity of the gene product thereof is the activity of XM_{—}473199-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “XM_{—}473199-protein”, especially from Oryza sativa or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said XM_{—}473199, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said XM_{—}473199, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said XM_{—}473199, or a functional equivalent or a homolog thereof as depicted in column 8 of Table II, application no. 2, preferably a homolog or functional equivalent as depicted in column 8 of Table II B, application no. 2, and being depicted in the same respective line as said XM_{—}473199, and preferably the activity is increased non-targeted.

Accordingly, in one embodiment, the molecule which activity is to be increased in the process of the invention is the gene product with an activity as a “XM_{—}473199-protein”, preferably it is the molecule of section (a) or (b) of this paragraph.

In particular, it was observed that in plants, especially in Arabidopsis thaliana, increasing or generating the activity of a gene product non-targeted with the activity of a “XM_{—}473199-protein”, preferably being encoded by a gene comprising the nucleic acid sequence SEQ ID NO. 62244, preferably the coding region thereof, conferred the production of or the increase in glutamine compared with the wild type control.

The nucleic acid sequence of Ybl021c from Saccharomyces cerevisiae, e.g. as shown in the respective line in column 5 of Table I, application no. 2, has been published in Goffeau et al., Science 274 (5287), 546 (1996). And the activity of the gene product thereof is the activity of transcription factor.

Accordingly, in one embodiment, the process of the present invention for producing proline in a non-human organism, like a microorganism or a plant or a part thereof, comprises increasing or generating the activity of a gene product with the activity of a gene product conferring the activity of “transcription factor”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Ybl021c, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Ybl021c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Ybl021c, or a functional equivalent or a homolog thereof as depicted in column 8 of Table II, application no. 2, preferably a homolog or functional equivalent as depicted in column 8 of Table II B, application no. 2, and being depicted in the same respective line as said Ybl021c, and preferably the activity is increased non-targeted.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 62524, preferably the coding region thereof, conferred the production of or the increase in proline compared with the wild type control.

The nucleic acid sequence of Ybr160w from Saccharomyces cerevisiae, e.g. as shown in the respective line in column 5 of Table I, application no. 2, has been published in Goffeau et al., Science 274 (5287), 546 (1996). And the activity of the gene product thereof is the activity of cell division control protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “cell division control protein”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Ybr160w, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Ybr160w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Ybr160w, or a functional equivalent or a homolog thereof as depicted in column 8 of Table II, application no. 2, preferably a homolog or functional equivalent as depicted in column 8 of Table II B, application no. 2, and being depicted in the same respective line as said Ybr160w, and preferably the activity is increased non-targeted.

Accordingly, in one embodiment, the molecule which activity is to be increased in the process of the invention is the gene product with an activity as a “cell division control protein”, preferably it is the molecule of section (a) or (b) of this paragraph.

In particular, it was observed that in plants, especially in Arabidopsis thaliana, increasing or generating the activity of a gene product non-targeted with the activity of a “cell division control protein”, preferably being encoded by a gene comprising the nucleic acid sequence SEQ ID NO. 62717, preferably the coding region thereof, conferred the production of or the increase in glutamate compared with the wild type control.

The nucleic acid sequence of Ybr160w from Saccharomyces cerevisiae, e.g. as shown in the respective line in column 5 of Table I, application no. 2, has been published in Goffeau et al., Science 274 (5287), 546 (1996). And the activity of the gene product thereof is the activity of cell division control protein.

Accordingly, in one embodiment, the process of the present invention for producing proline in a non-human organism, like a microorganism or a plant or a part thereof, comprises increasing or generating the activity of a gene product with the activity of a gene product conferring the activity of “cell division control protein”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Ybr160w, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Ybr160w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Ybr160w, or a functional equivalent or a homolog thereof as depicted in column 8 of Table II, application no. 2, preferably a homolog or functional equivalent as depicted in column 8 of Table II B, application no. 2, and being depicted in the same respective line as said Ybr160w, and preferably the activity is increased non-targeted.

Accordingly, in one embodiment, the molecule which activity is to be increased in the process of the invention is the gene product with an activity as a “cell division control protein”, preferably it is the molecule of section (a) or (b) of this paragraph.

In particular, it was observed that in plants, especially in Arabidopsis thaliana, increasing or generating the activity of a gene product non-targeted with the activity of a “cell division control protein”, preferably being encoded by a gene comprising the nucleic acid sequence SEQ ID NO. 62717, preferably the coding region thereof, conferred the production of or the increase in proline compared with the wild type control.

The nucleic acid sequence of Yc1026c-a from Saccharomyces cerevisiae, e.g. as shown in the respective line in column 5 of Table I, application no. 2, has been published in Goffeau et al., Science 274 (5287), 546 (1996). And the activity of the gene product thereof is the activity of YCL026C-A-protein.

Accordingly, in one embodiment, the process of the present invention for producing proline in a non-human organism, like a microorganism or a plant or a part thereof, comprises increasing or generating the activity of a gene product with the activity of a gene product conferring the activity of “YCL026C-A-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Yc1026c-a, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Yc1026c-a, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Yc1026c-a, or a functional equivalent or a homolog thereof as depicted in column 8 of Table II, application no. 2, preferably a homolog or functional equivalent as depicted in column 8 of Table II B, application no. 2, and being depicted in the same respective line as said Yc1026c-a, and preferably the activity is increased non-targeted.

Accordingly, in one embodiment, the molecule which activity is to be increased in the process of the invention is the gene product with an activity as a “YCL026C-A-protein”, preferably it is the molecule of section (a) or (b) of this paragraph.

In particular, it was observed that in plants, especially in Arabidopsis thaliana, increasing or generating the activity of a gene product non-targeted with the activity of a “YCL026C-A-protein”, preferably being encoded by a gene comprising the nucleic acid sequence SEQ ID NO. 63167, preferably the coding region thereof, conferred the production of or the increase in proline compared with the wild type control.

The nucleic acid sequence of Ycr102c from Saccharomyces cerevisiae, e.g. as shown in the respective line in column 5 of Table I, application no. 2, has been published in Goffeau et al., Science 274 (5287), 546 (1996). And the activity of the gene product thereof is the activity of ycr102c-protein.

Accordingly, in one embodiment, the process of the present invention for producing arginine in a non-human organism, like a microorganism or a plant or a part thereof, comprises increasing or generating the activity of a gene product with the activity of a gene product conferring the activity of “ycr102c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Ycr102c, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Ycr102c, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Ycr102c, or a functional equivalent or a homolog thereof as depicted in column 8 of Table II, application no. 2, preferably a homolog or functional equivalent as depicted in column 8 of Table II B, application no. 2, and being depicted in the same respective line as said Ycr102c, and preferably the activity is increased plastidic.

Accordingly, in one embodiment, the molecule which activity is to be increased in the process of the invention is the gene product with an activity as a “ycr102c-protein”, preferably it is the molecule of section (a) or (b) of this paragraph.

In particular, it was observed that in plants, especially in Arabidopsis thaliana, increasing or generating the activity of a gene product plastidic with the activity of a “ycr102c-protein”, preferably being encoded by a gene comprising the nucleic acid sequence SEQ ID NO. 63264, preferably the coding region thereof, conferred the production of or the increase in arginine compared with the wild type control.

The nucleic acid sequence of Ydl168w from Saccharomyces cerevisiae, e.g. as shown in the respective line in column 5 of Table I, application no. 2, has been published in Goffeau et al., Science 274 (5287), 546 (1996). And the activity of the gene product thereof is the activity of protein kinase.

Accordingly, in one embodiment, the process of the present invention for producing proline in a non-human organism, like a microorganism or a plant or a part thereof, comprises increasing or generating the activity of a gene product with the activity of a gene product conferring the activity of “protein kinase”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Ydl168w, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Ydl168w, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Ydl168w, or a functional equivalent or a homolog thereof as depicted in column 8 of Table II, application no. 2, preferably a homolog or functional equivalent as depicted in column 8 of Table II B, application no. 2, and being depicted in the same respective line as said Ydl168w, and preferably the activity is increased plastidic.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product plastidic with the activity of a “protein kinase”, preferably being encoded by a gene comprising the nucleic acid sequence SEQ ID NO. 14302, preferably the coding region thereof, conferred the production of or the increase in proline compared with the wild type control.

The nucleic acid sequence of Ydr044w from Saccharomyces cerevisiae, e.g. as shown in the respective line in column 5 of Table I, application no. 2, has been published in Goffeau et al., Science 274 (5287), 546 (1996). And the activity of the gene product thereof is the activity of coproporphyrinogen III oxidase.

Accordingly, in one embodiment, the process of the present invention for producing proline in a non-human organism, like a microorganism or a plant or a part thereof, comprises increasing or generating the activity of a gene product with the activity of a gene product conferring the activity of “coproporphyrinogen III oxidase”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Ydr044w, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Ydr044w, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Ydr044w, or a functional equivalent or a homolog thereof as depicted in column 8 of Table II, application no. 2, preferably a homolog or functional equivalent as depicted in column 8 of Table II B, application no. 2, and being depicted in the same respective line as said Ydr044w, and preferably the activity is increased plastidic.

Accordingly, in one embodiment, the molecule which activity is to be increased in the process of the invention is the gene product with an activity as a “coproporphyrinogen III oxidase”, preferably it is the molecule of section (a) or (b) of this paragraph.

In particular, it was observed that in plants, especially in Arabidopsis thaliana, increasing or generating the activity of a gene product plastidic with the activity of a “coproporphyrinogen III oxidase”, preferably being encoded by a gene comprising the nucleic acid sequence SEQ ID NO. 63334, preferably the coding region thereof, conferred the production of or the increase in proline compared with the wild type control.

The nucleic acid sequence of Ydr046c from Saccharomyces cerevisiae, e.g. as shown in the respective line in column 5 of Table I, application no. 2, has been published in Goffeau et al., Science 274 (5287), 546 (1996). And the activity of the gene product thereof is the activity of branched-chain amino acid permease.

Accordingly, in one embodiment, the process of the present invention for producing proline in a non-human organism, like a microorganism or a plant or a part thereof, comprises increasing or generating the activity of a gene product with the activity of a gene product conferring the activity of “branched-chain amino acid permease”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Ydr046c, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Ydr046c, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Ydr046c, or a functional equivalent or a homolog thereof as depicted in column 8 of Table II, application no. 2, preferably a homolog or functional equivalent as depicted in column 8 of Table II B, application no. 2, and being depicted in the same respective line as said Ydr046c, and preferably the activity is increased plastidic.

Accordingly, in one embodiment, the molecule which activity is to be increased in the process of the invention is the gene product with an activity as a “branched-chain amino acid permease”, preferably it is the molecule of section (a) or (b) of this paragraph.

In particular, it was observed that in plants, especially in Arabidopsis thaliana, increasing or generating the activity of a gene product plastidic with the activity of a “branched-chain amino acid permease”, preferably being encoded by a gene comprising the nucleic acid sequence SEQ ID NO. 63544, preferably the coding region thereof, conferred the production of or the increase in proline compared with the wild type control.

The nucleic acid sequence of Ydr265w from Saccharomyces cerevisiae, e.g. as shown in the respective line in column 5 of Table I, application no. 2, has been published in Goffeau et al., Science 274 (5287), 546 (1996). And the activity of the gene product thereof is the activity of peroxisome assembly protein.

Accordingly, in one embodiment, the process of the present invention for producing arginine in a non-human organism, like a microorganism or a plant or a part thereof, comprises increasing or generating the activity of a gene product with the activity of a gene product conferring the activity of “peroxisome assembly protein”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Ydr265w, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Ydr265w, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Ydr265w, or a functional equivalent or a homolog thereof as depicted in column 8 of Table II, application no. 2, preferably a homolog or functional equivalent as depicted in column 8 of Table II B, application no. 2, and being depicted in the same respective line as said Ydr265w, and preferably the activity is increased plastidic.

Accordingly, in one embodiment, the molecule which activity is to be increased in the process of the invention is the gene product with an activity as a “peroxisome assembly protein”, preferably it is the molecule of section (a) or (b) of this paragraph.

In particular, it was observed that in plants, especially in Arabidopsis thaliana, increasing or generating the activity of a gene product plastidic with the activity of a “peroxisome assembly protein”, preferably being encoded by a gene comprising the nucleic acid sequence SEQ ID NO. 63665, preferably the coding region thereof, conferred the production of or the increase in arginine compared with the wild type control.

The nucleic acid sequence of Ydr338c from Saccharomyces cerevisiae, e.g. as shown in the respective line in column 5 of Table I, application no. 2, has been published in Goffeau et al., Science 274 (5287), 546 (1996). And the activity of the gene product thereof is the activity of ydr338c-protein.

Accordingly, in one embodiment, the process of the present invention for producing arginine in a non-human organism, like a microorganism or a plant or a part thereof, comprises increasing or generating the activity of a gene product with the activity of a gene product conferring the activity of “ydr338c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Ydr338c, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Ydr338c, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Ydr338c, or a functional equivalent or a homolog thereof as depicted in column 8 of Table II, application no. 2, preferably a homolog or functional equivalent as depicted in column 8 of Table II B, application no. 2, and being depicted in the same respective line as said Ydr338c, and preferably the activity is increased plastidic.

Accordingly, in one embodiment, the molecule which activity is to be increased in the process of the invention is the gene product with an activity as a “ydr338c-protein”, preferably it is the molecule of section (a) or (b) of this paragraph.

In particular, it was observed that in plants, especially in Arabidopsis thaliana, increasing or generating the activity of a gene product plastidic with the activity of a “ydr338c-protein”, preferably being encoded by a gene comprising the nucleic acid sequence SEQ ID NO. 63713, preferably the coding region thereof, conferred the production of or the increase in arginine compared with the wild type control.

The nucleic acid sequence of Yer014w from Saccharomyces cerevisiae, e.g. as shown in the respective line in column 5 of Table I, application no. 2, has been published in Goffeau et al., Science 274 (5287), 546 (1996). And the activity of the gene product thereof is the activity of yer014w-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “yer014w-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Yer014w, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Yer014w, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Yer014w, or a functional equivalent or a homolog thereof as depicted in column 8 of Table II, application no. 2, preferably a homolog or functional equivalent as depicted in column 8 of Table II B, application no. 2, and being depicted in the same respective line as said Yer014w, and preferably the activity is increased plastidic.

Accordingly, in one embodiment, the molecule which activity is to be increased in the process of the invention is the gene product with an activity as a “yer014w-protein”, preferably it is the molecule of section (a) or (b) of this paragraph.

In particular, it was observed that in plants, especially in Arabidopsis thaliana, increasing or generating the activity of a gene product plastidic with the activity of a “yer014w-protein”, preferably being encoded by a gene comprising the nucleic acid sequence SEQ ID NO. 63745, preferably the coding region thereof, conferred the production of or the increase in glutamine compared with the wild type control.

The nucleic acid sequence of Yer106w from Saccharomyces cerevisiae, e.g. as shown in the respective line in column 5 of Table I, application no. 2, has been published in Goffeau et al., Science 274 (5287), 546 (1996). And the activity of the gene product thereof is the activity of yer106w-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “yer106w-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Yer106w, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Yer106w, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Yer106w, or a functional equivalent or a homolog thereof as depicted in column 8 of Table II, application no. 2, preferably a homolog or functional equivalent as depicted in column 8 of Table II B, application no. 2, and being depicted in the same respective line as said Yer106w, and preferably the activity is increased plastidic.

Accordingly, in one embodiment, the molecule which activity is to be increased in the process of the invention is the gene product with an activity as a “yer106w-protein”, preferably it is the molecule of section (a) or (b) of this paragraph.

In particular, it was observed that in plants, especially in Arabidopsis thaliana, increasing or generating the activity of a gene product plastidic with the activity of a “yer106w-protein”, preferably being encoded by a gene comprising the nucleic acid sequence SEQ ID NO. 63803, preferably the coding region thereof, conferred the production of or the increase in glutamine compared with the wild type control.

The nucleic acid sequence of Yfl016c from Saccharomyces cerevisiae, e.g. as shown in the respective line in column 5 of Table I, application no. 2, has been published in Goffeau et al., Science 274 (5287), 546 (1996). And the activity of the gene product thereof is the activity of molecular chaperone portein.

Accordingly, in one embodiment, the process of the present invention for producing proline in a non-human organism, like a microorganism or a plant or a part thereof, comprises increasing or generating the activity of a gene product with the activity of a gene product conferring the activity of “molecular chaperone portein”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Yfl016c, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Yfl016c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Yfl016c, or a functional equivalent or a homolog thereof as depicted in column 8 of Table II, application no. 2, preferably a homolog or functional equivalent as depicted in column 8 of Table II B, application no. 2, and being depicted in the same respective line as said Yfl016c, and preferably the activity is increased non-targeted.

Accordingly, in one embodiment, the molecule which activity is to be increased in the process of the invention is the gene product with an activity as a “molecular chaperone portein”, preferably it is the molecule of section (a) or (b) of this paragraph. In particular, it was observed that in plants, especially in Arabidopsis thaliana, increasing or generating the activity of a gene product non-targeted with the activity of a “molecular chaperone portein”, preferably being encoded by a gene comprising the nucleic acid sequence SEQ ID NO. 63807, preferably the coding region thereof, conferred the production of or the increase in proline compared with the wild type control.

The nucleic acid sequence of Yfl019c from Saccharomyces cerevisiae, e.g. as shown in the respective line in column 5 of Table I, application no. 2, has been published in Goffeau et al., Science 274 (5287), 546 (1996). And the activity of the gene product thereof is the activity of YFL019C-protein.

Accordingly, in one embodiment, the process of the present invention for producing proline in a non-human organism, like a microorganism or a plant or a part thereof, comprises increasing or generating the activity of a gene product with the activity of a gene product conferring the activity of “YFL0190-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Yfl019c, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Yfl019c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Yfl019c, or a functional equivalent or a homolog thereof as depicted in column 8 of Table II, application no. 2, preferably a homolog or functional equivalent as depicted in column 8 of Table II B, application no. 2, and being depicted in the same respective line as said Yfl019c, and preferably the activity is increased non-targeted.

Accordingly, in one embodiment, the molecule which activity is to be increased in the process of the invention is the gene product with an activity as a “YFL0190-protein”, preferably it is the molecule of section (a) or (b) of this paragraph.

In particular, it was observed that in plants, especially in Arabidopsis thaliana, increasing or generating the activity of a gene product non-targeted with the activity of a “YFL019C-protein”, preferably being encoded by a gene comprising the nucleic acid sequence SEQ ID NO. 64144, preferably the coding region thereof, conferred the production of or the increase in proline compared with the wild type control.

The nucleic acid sequence of Yfl054c from Saccharomyces cerevisiae, e.g. as shown in the respective line in column 5 of Table I, application no. 2, has been published in Goffeau et al., Science 274 (5287), 546 (1996). And the activity of the gene product thereof is the activity of yf1054c-protein.

Accordingly, in one embodiment, the process of the present invention for producing arginine in a non-human organism, like a microorganism or a plant or a part thereof, comprises increasing or generating the activity of a gene product with the activity of a gene product conferring the activity of “yf1054c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Yfl054c, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Yfl054c, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Yfl054c, or a functional equivalent or a homolog thereof as depicted in column 8 of Table II, application no. 2, preferably a homolog or functional equivalent as depicted in column 8 of Table II B, application no. 2, and being depicted in the same respective line as said

Yfl054c, and preferably the activity is increased plastidic.

Accordingly, in one embodiment, the molecule which activity is to be increased in the process of the invention is the gene product with an activity as a “yf1054c-protein”, preferably it is the molecule of section (a) or (b) of this paragraph.

In particular, it was observed that in plants, especially in Arabidopsis thaliana, increasing or generating the activity of a gene product plastidic with the activity of a “yf1054c-protein”, preferably being encoded by a gene comprising the nucleic acid sequence SEQ ID NO. 64148, preferably the coding region thereof, conferred the production of or the increase in arginine compared with the wild type control.

The nucleic acid sequence of Yfl054c from Saccharomyces cerevisiae, e.g. as shown in the respective line in column 5 of Table I, application no. 2, has been published in Goffeau et al., Science 274 (5287), 546 (1996). And the activity of the gene product thereof is the activity of yf1054c-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “yf1054c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Yfl054c, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Yfl054c, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Yfl054c, or a functional equivalent or a homolog thereof as depicted in column 8 of Table II, application no. 2, preferably a homolog or functional equivalent as depicted in column 8 of Table II B, application no. 2, and being depicted in the same respective line as said Yfl054c, and preferably the activity is increased plastidic.

Accordingly, in one embodiment, the molecule which activity is to be increased in the process of the invention is the gene product with an activity as a “yf1054c-protein”, preferably it is the molecule of section (a) or (b) of this paragraph.

In particular, it was observed that in plants, especially in Arabidopsis thaliana, increasing or generating the activity of a gene product plastidic with the activity of a “yf1054c-protein”, preferably being encoded by a gene comprising the nucleic acid sequence SEQ ID NO. 64148, preferably the coding region thereof, conferred the production of or the increase in glutamine compared with the wild type control.

The nucleic acid sequence of Yg1174w from Saccharomyces cerevisiae, e.g. as shown in the respective line in column 5 of Table I, application no. 2, has been published in Goffeau et al., Science 274 (5287), 546 (1996). And the activity of the gene product thereof is the activity of pre-mRNA-splicing factor.

Accordingly, in one embodiment, the process of the present invention for producing arginine in a non-human organism, like a microorganism or a plant or a part thereof, comprises increasing or generating the activity of a gene product with the activity of a gene product conferring the activity of “pre-mRNA-splicing factor”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Yg1174w, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Yg1174w, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Yg1174w, or a functional equivalent or a homolog thereof as depicted in column 8 of Table II, application no. 2, preferably a homolog or functional equivalent as depicted in column 8 of Table II B, application no. 2, and being depicted in the same respective line as said Yg1174w, and preferably the activity is increased plastidic.

Accordingly, in one embodiment, the molecule which activity is to be increased in the process of the invention is the gene product with an activity as a “pre-mRNA-splicing factor”, preferably it is the molecule of section (a) or (b) of this paragraph.

In particular, it was observed that in plants, especially in Arabidopsis thaliana, increasing or generating the activity of a gene product plastidic with the activity of a “pre-mRNA-splicing factor”, preferably being encoded by a gene comprising the nucleic acid sequence SEQ ID NO. 64157, preferably the coding region thereof, conferred the production of or the increase in arginine compared with the wild type control.

The nucleic acid sequence of Yg1237c from Saccharomyces cerevisiae, e.g. as shown in the respective line in column 5 of Table I, application no. 2, has been published in Goffeau et al., Science 274 (5287), 546 (1996). And the activity of the gene product thereof is the activity of yg1237c-protein.

Accordingly, in one embodiment, the process of the present invention for producing proline in a non-human organism, like a microorganism or a plant or a part thereof, comprises increasing or generating the activity of a gene product with the activity of a gene product conferring the activity of “yg1237c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Yg1237c, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Yg1237c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Yg1237c, or a functional equivalent or a homolog thereof as depicted in column 8 of Table II, application no. 2, preferably a homolog or functional equivalent as depicted in column 8 of Table II B, application no. 2, and being depicted in the same respective line as said Yg1237c, and preferably the activity is increased non-targeted,.

Accordingly, in one embodiment, the molecule which activity is to be increased in the process of the invention is the gene product with an activity as a “yg1237c-protein”, preferably it is the molecule of section (a) or (b) of this paragraph.

In particular, it was observed that in plants, especially in Arabidopsis thaliana, increasing or generating the activity of a gene product non-targeted with the activity of a “yg1237c-protein”, preferably being encoded by a gene comprising the nucleic acid sequence SEQ ID NO. 64177, preferably the coding region thereof, conferred the production of or the increase in proline compared with the wild type control.

The nucleic acid sequence of Ygr068c from Saccharomyces cerevisiae, e.g. as shown in the respective line in column 5 of Table I, application no. 2, has been published in Goffeau et al., Science 274 (5287), 546 (1996). And the activity of the gene product thereof is the activity of ygr068c-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “ygr068c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Ygr068c, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Ygr068c, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Ygr068c, or a functional equivalent or a homolog thereof as depicted in column 8 of Table II, application no. 2, preferably a homolog or functional equivalent as depicted in column 8 of Table II B, application no. 2, and being depicted in the same respective line as said Ygr068c, and preferably the activity is increased plastidic.

Accordingly, in one embodiment, the molecule which activity is to be increased in the process of the invention is the gene product with an activity as a “ygr068c-protein”, preferably it is the molecule of section (a) or (b) of this paragraph.

In particular, it was observed that in plants, especially in Arabidopsis thaliana, increasing or generating the activity of a gene product plastidic with the activity of a “ygr068c-protein”, preferably being encoded by a gene comprising the nucleic acid sequence SEQ ID NO. 64198, preferably the coding region thereof, conferred the production of or the increase in glutamine compared with the wild type control.

The nucleic acid sequence of Ygr175c from Saccharomyces cerevisiae, e.g. as shown in the respective line in column 5 of Table I, application no. 2, has been published in Goffeau et al., Science 274 (5287), 546 (1996). And the activity of the gene product thereof is the activity of squalene monooxygenase.

Accordingly, in one embodiment, the process of the present invention for producing proline in a non-human organism, like a microorganism or a plant or a part thereof, comprises increasing or generating the activity of a gene product with the activity of a gene product conferring the activity of “squalene monooxygenase”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Ygr175c, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Ygr175c, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Ygr175c, or a functional equivalent or a homolog thereof as depicted in column 8 of Table II, application no. 2, preferably a homolog or functional equivalent as depicted in column 8 of Table II B, application no. 2, and being depicted in the same respective line as said Ygr175c, and preferably the activity is increased plastidic.

Accordingly, in one embodiment, the molecule which activity is to be increased in the process of the invention is the gene product with an activity as a “squalene monooxygenase”, preferably it is the molecule of section (a) or (b) of this paragraph.

In particular, it was observed that in plants, especially in Arabidopsis thaliana, increasing or generating the activity of a gene product plastidic with the activity of a “squalene monooxygenase”, preferably being encoded by a gene comprising the nucleic acid sequence SEQ ID NO. 64218, preferably the coding region thereof, conferred the production of or the increase in proline compared with the wild type control.

The nucleic acid sequence of Ygr221c from Saccharomyces cerevisiae, e.g. as shown in the respective line in column 5 of Table I, application no. 2, has been published in Goffeau et al., Science 274 (5287), 546 (1996). And the activity of the gene product thereof is the activity of ygr221c-protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “ygr221c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Ygr221c, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Ygr221c, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Ygr221c, or a functional equivalent or a homolog thereof as depicted in column 8 of Table II, application no. 2, preferably a homolog or functional equivalent as depicted in column 8 of Table II B, application no. 2, and being depicted in the same respective line as said Ygr221c, and preferably the activity is increased plastidic.

Accordingly, in one embodiment, the molecule which activity is to be increased in the process of the invention is the gene product with an activity as a “ygr221c-protein”, preferably it is the molecule of section (a) or (b) of this paragraph.

In particular, it was observed that in plants, especially in Arabidopsis thaliana, increasing or generating the activity of a gene product plastidic with the activity of a “ygr221c-protein”, preferably being encoded by a gene comprising the nucleic acid sequence SEQ ID NO. 64315, preferably the coding region thereof, conferred the production of or the increase in glutamate compared with the wild type control.

The nucleic acid sequence of Ygr221c from Saccharomyces cerevisiae, e.g. as shown in the respective line in column 5 of Table I, application no. 2, has been published in Goffeau et al., Science 274 (5287), 546 (1996). And the activity of the gene product thereof is the activity of ygr221c-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “ygr221c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Ygr221c, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Ygr221c, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Ygr221c, or a functional equivalent or a homolog thereof as depicted in column 8 of Table II, application no. 2, preferably a homolog or functional equivalent as depicted in column 8 of Table II B, application no. 2, and being depicted in the same respective line as said Ygr221c, and preferably the activity is increased plastidic.

Accordingly, in one embodiment, the molecule which activity is to be increased in the process of the invention is the gene product with an activity as a “ygr221c-protein”, preferably it is the molecule of section (a) or (b) of this paragraph.

In particular, it was observed that in plants, especially in Arabidopsis thaliana, increasing or generating the activity of a gene product plastidic with the activity of a “ygr221c-protein”, preferably being encoded by a gene comprising the nucleic acid sequence SEQ ID NO. 64315, preferably the coding region thereof, conferred the production of or the increase in glutamine compared with the wild type control.

The nucleic acid sequence of Yhl013c from Saccharomyces cerevisiae, e.g. as shown in the respective line in column 5 of Table I, application no. 2, has been published in Goffeau et al., Science 274 (5287), 546 (1996). And the activity of the gene product thereof is the activity of yhl013c-protein.

Accordingly, in one embodiment, the process of the present invention for producing proline in a non-human organism, like a microorganism or a plant or a part thereof, comprises increasing or generating the activity of a gene product with the activity of a gene product conferring the activity of “yhl013c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Yhl013c, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Yhl013c, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Yhl013c, or a functional equivalent or a homolog thereof as depicted in column 8 of Table II, application no. 2, preferably a homolog or functional equivalent as depicted in column 8 of Table II B, application no. 2, and being depicted in the same respective line as said Yhl013c, and preferably the activity is increased plastidic.

Accordingly, in one embodiment, the molecule which activity is to be increased in the process of the invention is the gene product with an activity as a “yhl013c-protein”, preferably it is the molecule of section (a) or (b) of this paragraph.

In particular, it was observed that in plants, especially in Arabidopsis thaliana, increasing or generating the activity of a gene product plastidic with the activity of a “yhl013c-protein”, preferably being encoded by a gene comprising the nucleic acid sequence SEQ ID NO. 14715, preferably the coding region thereof, conferred the production of or the increase in proline compared with the wild type control.

The nucleic acid sequence of Yhr024c from Saccharomyces cerevisiae, e.g. as shown in the respective line in column 5 of Table I, application no. 2, has been published in Goffeau et al., Science 274 (5287), 546 (1996). And the activity of the gene product thereof is the activity of mitochondrial processing protease.

Accordingly, in one embodiment, the process of the present invention for producing arginine in a non-human organism, like a microorganism or a plant or a part thereof, comprises increasing or generating the activity of a gene product with the activity of a gene product conferring the activity of “mitochondrial processing protease”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Yhr024c, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Yhr024c, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Yhr024c, or a functional equivalent or a homolog thereof as depicted in column 8 of Table II, application no. 2, preferably a homolog or functional equivalent as depicted in column 8 of Table II B, application no. 2, and being depicted in the same respective line as said Yhr024c, and preferably the activity is increased plastidic.

Accordingly, in one embodiment, the molecule which activity is to be increased in the process of the invention is the gene product with an activity as a “mitochondrial processing protease”, preferably it is the molecule of section (a) or (b) of this paragraph.

In particular, it was observed that in plants, especially in Arabidopsis thaliana, increasing or generating the activity of a gene product plastidic with the activity of a “mitochondrial processing protease”, preferably being encoded by a gene comprising the nucleic acid sequence SEQ ID NO. 64336, preferably the coding region thereof, conferred the production of or the increase in arginine compared with the wild type control.

The nucleic acid sequence of Yhr072w-a from Saccharomyces cerevisiae, e.g. as shown in the respective line in column 5 of Table I, application no. 2, has been published in Goffeau et al., Science 274 (5287), 546 (1996). And the activity of the gene product thereof is the activity of H/ACA ribonucleoprotein complex subunit 3.

Accordingly, in one embodiment, the process of the present invention for producing proline in a non-human organism, like a microorganism or a plant or a part thereof, comprises increasing or generating the activity of a gene product with the activity of a gene product conferring the activity of “H/ACA ribonucleoprotein complex subunit 3”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Yhr072w-a, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Yhr072w-a, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Yhr072w-a, or a functional equivalent or a homolog thereof as depicted in column 8 of Table II, application no. 2, preferably a homolog or functional equivalent as depicted in column 8 of Table II B, application no. 2, and being depicted in the same respective line as said Yhr072w-a, and preferably the activity is increased non-targeted.

Accordingly, in one embodiment, the molecule which activity is to be increased in the process of the invention is the gene product with an activity as a “H/ACA ribonucleoprotein complex subunit 3”, preferably it is the molecule of section (a) or (b) of this paragraph.

In particular, it was observed that in plants, especially in Arabidopsis thaliana, increasing or generating the activity of a gene product non-targeted with the activity of a “H/ACA ribonucleoprotein complex subunit 3”, preferably being encoded by a gene comprising the nucleic acid sequence SEQ ID NO. 64470, preferably the coding region thereof, conferred the production of or the increase in proline compared with the wild type control.

The nucleic acid sequence of Yhr207c from Saccharomyces cerevisiae, e.g. as shown in the respective line in column 5 of Table I, application no. 2, has been published in Goffeau et al., Science 274 (5287), 546 (1996). And the activity of the gene product thereof is the activity of yhr207c-protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “yhr207c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Yhr207c, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Yhr207c, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Yhr207c, or a functional equivalent or a homolog thereof as depicted in column 8 of Table II, application no. 2, preferably a homolog or functional equivalent as depicted in column 8 of Table II B, application no. 2, and being depicted in the same respective line as said Yhr207c, and preferably the activity is increased plastidic.

Accordingly, in one embodiment, the molecule which activity is to be increased in the process of the invention is the gene product with an activity as a “yhr207c-protein”, preferably it is the molecule of section (a) or (b) of this paragraph.

In particular, it was observed that in plants, especially in Arabidopsis thaliana, increasing or generating the activity of a gene product plastidic with the activity of a “yhr207c-protein”, preferably being encoded by a gene comprising the nucleic acid sequence SEQ ID NO. 64546, preferably the coding region thereof, conferred the production of or the increase in glutamate compared with the wild type control.

The nucleic acid sequence of Yil074c from Saccharomyces cerevisiae, e.g. as shown in the respective line in column 5 of Table I, application no. 2, has been published in Goffeau et al., Science 274 (5287), 546 (1996). And the activity of the gene product thereof is the activity of 3-phosphoglycerate dehydrogenase.

Accordingly, in one embodiment, the process of the present invention for producing arginine in a non-human organism, like a microorganism or a plant or a part thereof, comprises increasing or generating the activity of a gene product with the activity of a gene product conferring the activity of “3-phosphoglycerate dehydrogenase”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Yil074c, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Yil074c, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Yil074c, or a functional equivalent or a homolog thereof as depicted in column 8 of Table II, application no. 2, preferably a homolog or functional equivalent as depicted in column 8 of Table II B, application no. 2, and being depicted in the same respective line as said Yil074c, and preferably the activity is increased plastidic.

Accordingly, in one embodiment, the molecule which activity is to be increased in the process of the invention is the gene product with an activity as a “3-phosphoglycerate dehydrogenase”, preferably it is the molecule of section (a) or (b) of this paragraph.

In particular, it was observed that in plants, especially in Arabidopsis thaliana, increasing or generating the activity of a gene product plastidic with the activity of a “3-phosphoglycerate dehydrogenase”, preferably being encoded by a gene comprising the nucleic acid sequence SEQ ID NO. 64563, preferably the coding region thereof, conferred the production of or the increase in arginine compared with the wild type control.

The nucleic acid sequence of Yir027c from Saccharomyces cerevisiae, e.g. as shown in the respective line in column 5 of Table I, application no. 2, has been published in Goffeau et al., Science 274 (5287), 546 (1996). And the activity of the gene product thereof is the activity of allantoinase.

Accordingly, in one embodiment, the process of the present invention for producing proline in a non-human organism, like a microorganism or a plant or a part thereof, comprises increasing or generating the activity of a gene product with the activity of a gene product conferring the activity of “allantoinase”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Yir027c, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Yir027c, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Yir027c, or a functional equivalent or a homolog thereof as depicted in column 8 of Table II, application no. 2, preferably a homolog or functional equivalent as depicted in column 8 of Table II B, application no. 2, and being depicted in the same respective line as said Yir027c, and preferably the activity is increased plastidic.

Accordingly, in one embodiment, the molecule which activity is to be increased in the process of the invention is the gene product with an activity as a “allantoinase”, preferably it is the molecule of section (a) or (b) of this paragraph.

In particular, it was observed that in plants, especially in Arabidopsis thaliana, increasing or generating the activity of a gene product plastidic with the activity of a “allantoinase”, preferably being encoded by a gene comprising the nucleic acid sequence SEQ ID NO. 64773, preferably the coding region thereof, conferred the production of or the increase in proline compared with the wild type control.

The nucleic acid sequence of Yir029w from Saccharomyces cerevisiae, e.g. as shown in the respective line in column 5 of Table I, application no. 2, has been published in Goffeau et al., Science 274 (5287), 546 (1996). And the activity of the gene product thereof is the activity of allantoicase.

Accordingly, in one embodiment, the process of the present invention for producing arginine in a non-human organism, like a microorganism or a plant or a part thereof, comprises increasing or generating the activity of a gene product with the activity of a gene product conferring the activity of “allantoicase”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Yir029w, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Yir029w, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Yir029w, or a functional equivalent or a homolog thereof as depicted in column 8 of Table II, application no. 2, preferably a homolog or functional equivalent as depicted in column 8 of Table II B, application no. 2, and being depicted in the same respective line as said Yir029w, and preferably the activity is increased plastidic.

Accordingly, in one embodiment, the molecule which activity is to be increased in the process of the invention is the gene product with an activity as a “allantoicase”, preferably it is the molecule of section (a) or (b) of this paragraph.

In particular, it was observed that in plants, especially in Arabidopsis thaliana, increasing or generating the activity of a gene product plastidic with the activity of a “allantoicase”, preferably being encoded by a gene comprising the nucleic acid sequence SEQ ID NO. 64894, preferably the coding region thereof, conferred the production of or the increase in arginine compared with the wild type control.

The nucleic acid sequence of Yjl073w from Saccharomyces cerevisiae, e.g. as shown in the respective line in column 5 of Table I, application no. 2, has been published in Goffeau et al., Science 274 (5287), 546 (1996). And the activity of the gene product thereof is the activity of DnaJ-like chaperone.

Accordingly, in one embodiment, the process of the present invention for producing glutamine in a non-human organism, like a microorganism or a plant or a part thereof, comprises increasing or generating the activity of a gene product with the activity of a gene product conferring the activity of “DnaJ-like chaperone”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Yjl073w, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Yjl073w, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Yjl073w, or a functional equivalent or a homolog thereof as depicted in column 8 of Table II, application no. 2, preferably a homolog or functional equivalent as depicted in column 8 of Table II B, application no. 2, and being depicted in the same respective line as said Yjl073w, and preferably the activity is increased plastidic.

Accordingly, in one embodiment, the molecule which activity is to be increased in the process of the invention is the gene product with an activity as a “DnaJ-like chaperone”, preferably it is the molecule of section (a) or (b) of this paragraph.

In particular, it was observed that in plants, especially in Arabidopsis thaliana, increasing or generating the activity of a gene product plastidic with the activity of a “DnaJ-like chaperone”, preferably being encoded by a gene comprising the nucleic acid sequence SEQ ID NO. 64964, preferably the coding region thereof, conferred the production of or the increase in glutamine compared with the wild type control.

The nucleic acid sequence of Yjl088w from Saccharomyces cerevisiae, e.g. as shown in the respective line in column 5 of Table I, application no. 2, has been published in Goffeau et al., Science 274 (5287), 546 (1996). And the activity of the gene product thereof is the activity of ornithine carbamoyltransferase.

Accordingly, in one embodiment, the process of the present invention for producing arginine in a non-human organism, like a microorganism or a plant or a part thereof, comprises increasing or generating the activity of a gene product with the activity of a gene product conferring the activity of “ornithine carbamoyltransferase”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Yjl088w, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Yjl088w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Yjl088w, or a functional equivalent or a homolog thereof as depicted in column 8 of Table II, application no. 2, preferably a homolog or functional equivalent as depicted in column 8 of Table II B, application no. 2, and being depicted in the same respective line as said Yjl088w, and preferably the activity is increased non-targeted.

Accordingly, in one embodiment, the molecule which activity is to be increased in the process of the invention is the gene product with an activity as a “ornithine carbamoyltransferase”, preferably it is the molecule of section (a) or (b) of this paragraph.

In particular, it was observed that in plants, especially in Arabidopsis thaliana, increasing or generating the activity of a gene product non-targeted with the activity of a “ornithine carbamoyltransferase”, preferably being encoded by a gene comprising the nucleic acid sequence SEQ ID NO. 64975, preferably the coding region thereof, conferred the production of or the increase in arginine compared with the wild type control.

The nucleic acid sequence of Yjl129c from Saccharomyces cerevisiae, e.g. as shown in the respective line in column 5 of Table I, application no. 2, has been published in Goffeau et al., Science 274 (5287), 546 (1996). And the activity of the gene product thereof is the activity of potassium transport protein.

Accordingly, in one embodiment, the process of the present invention for producing proline in a non-human organism, like a microorganism or a plant or a part thereof, comprises increasing or generating the activity of a gene product with the activity of a gene product conferring the activity of “potassium transport protein”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Yjl129c, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Yjl129c, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Yjl129c, or a functional equivalent or a homolog thereof as depicted in column 8 of Table II, application no. 2, preferably a homolog or functional equivalent as depicted in column 8 of Table II B, application no. 2, and being depicted in the same respective line as said Yjl129c, and preferably the activity is increased plastidic.

Accordingly, in one embodiment, the molecule which activity is to be increased in the process of the invention is the gene product with an activity as a “potassium transport protein”, preferably it is the molecule of section (a) or (b) of this paragraph.

In particular, it was observed that in plants, especially in Arabidopsis thaliana, increasing or generating the activity of a gene product plastidic with the activity of a “potassium transport protein”, preferably being encoded by a gene comprising the nucleic acid sequence SEQ ID NO. 65181, preferably the coding region thereof, conferred the production of or the increase in proline compared with the wild type control.

The nucleic acid sequence of Yjl137c from Saccharomyces cerevisiae, e.g. as shown in the respective line in column 5 of Table I, application no. 2, has been published in Goffeau et al., Science 274 (5287), 546 (1996). And the activity of the gene product thereof is the activity of glycogenin.

Accordingly, in one embodiment, the process of the present invention for producing arginine in a non-human organism, like a microorganism or a plant or a part thereof, comprises increasing or generating the activity of a gene product with the activity of a gene product conferring the activity of “glycogenin”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said YjI137c, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Yjl137c, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Yjl137c, or a functional equivalent or a homolog thereof as depicted in column 8 of Table II, application no. 2, preferably a homolog or functional equivalent as depicted in column 8 of Table II B, application no. 2, and being depicted in the same respective line as said Yjl137c, and preferably the activity is increased plastidic.

Accordingly, in one embodiment, the molecule which activity is to be increased in the process of the invention is the gene product with an activity as a “glycogenin”, preferably it is the molecule of section (a) or (b) of this paragraph.

In particular, it was observed that in plants, especially in Arabidopsis thaliana, increasing or generating the activity of a gene product plastidic with the activity of a “glycogenin”, preferably being encoded by a gene comprising the nucleic acid sequence SEQ ID NO. 14821, preferably the coding region thereof, conferred the production of or the increase in arginine compared with the wild type control.

The nucleic acid sequence of Yjl138c from Saccharomyces cerevisiae, e.g. as shown in the respective line in column 5 of Table I, application no. 2, has been published in Goffeau et al., Science 274 (5287), 546 (1996). And the activity of the gene product thereof is the activity of ATP-dependent RNA helicase.

Accordingly, in one embodiment, the process of the present invention for producing proline in a non-human organism, like a microorganism or a plant or a part thereof, comprises increasing or generating the activity of a gene product with the activity of a gene product conferring the activity of “ATP-dependent RNA helicase”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said YjI138c, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said YjI138c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said YjI138c, or a functional equivalent or a homolog thereof as depicted in column 8 of Table II, application no. 2, preferably a homolog or functional equivalent as depicted in column 8 of Table II B, application no. 2, and being depicted in the same respective line as said YjI138c, and preferably the activity is increased non-targeted.

Accordingly, in one embodiment, the molecule which activity is to be increased in the process of the invention is the gene product with an activity as a “ATP-dependent RNA helicase”, preferably it is the molecule of section (a) or (b) of this paragraph.

In particular, it was observed that in plants, especially in Arabidopsis thaliana, increasing or generating the activity of a gene product non-targeted with the activity of a “ATP-dependent RNA helicase”, preferably being encoded by a gene comprising the nucleic acid sequence SEQ ID NO. 65224, preferably the coding region thereof, conferred the production of or the increase in proline compared with the wild type control.

The nucleic acid sequence of Yjr133w from Saccharomyces cerevisiae, e.g. as shown in the respective line in column 5 of Table I, application no. 2, has been published in Goffeau et al., Science 274 (5287), 546 (1996). And the activity of the gene product thereof is the activity of phosphoribosyltransferase.

Accordingly, in one embodiment, the process of the present invention for producing glutamine in a non-human organism, like a microorganism or a plant or a part thereof, comprises increasing or generating the activity of a gene product with the activity of a gene product conferring the activity of “phosphoribosyltransferase”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Yjr133w, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Yjr133w, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Yjr133w, or a functional equivalent or a homolog thereof as depicted in column 8 of Table II, application no. 2, preferably a homolog or functional equivalent as depicted in column 8 of Table II B, application no. 2, and being depicted in the same respective line as said Yjr133w, and preferably the activity is increased plastidic.

Accordingly, in one embodiment, the molecule which activity is to be increased in the process of the invention is the gene product with an activity as a “phosphoribosyltransferase”, preferably it is the molecule of section (a) or (b) of this paragraph.

In particular, it was observed that in plants, especially in Arabidopsis thaliana, increasing or generating the activity of a gene product plastidic with the activity of a “phosphoribosyltransferase”, preferably being encoded by a gene comprising the nucleic acid sequence SEQ ID NO. 66225, preferably the coding region thereof, conferred the production of or the increase in glutamine compared with the wild type control.

The nucleic acid sequence of Yjr153w from Saccharomyces cerevisiae, e.g. as shown in the respective line in column 5 of Table I, application no. 2, has been published in Goffeau et al., Science 274 (5287), 546 (1996). And the activity of the gene product thereof is the activity of polygalacturonase.

Accordingly, in one embodiment, the process of the present invention for producing glutamate in a non-human organism, like a microorganism or a plant or a part thereof, comprises increasing or generating the activity of a gene product with the activity of a gene product conferring the activity of “polygalacturonase”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Yjr153w, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Yjr153w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Yjr153w, or a functional equivalent or a homolog thereof as depicted in column 8 of Table II, application no. 2, preferably a homolog or functional equivalent as depicted in column 8 of Table II B, application no. 2, and being depicted in the same respective line as said Yjr153w, and preferably the activity is increased non-targeted.

Accordingly, in one embodiment, the molecule which activity is to be increased in the process of the invention is the gene product with an activity as a “polygalacturonase”, preferably it is the molecule of section (a) or (b) of this paragraph.
In particular, it was observed that in plants, especially in Arabidopsis thaliana, increasing or generating the activity of a gene product non-targeted with the activity of a “polygalacturonase”, preferably being encoded by a gene comprising the nucleic acid sequence SEQ ID NO. 66274, preferably the coding region thereof, conferred the production of or the increase in glutamate compared with the wild type control.

The nucleic acid sequence of Ykl106w from Saccharomyces cerevisiae, e.g. as shown in the respective line in column 5 of Table I, application no. 2, has been published in Goffeau et al., Science 274 (5287), 546 (1996). And the activity of the gene product thereof is the activity of aspartate aminotransferase.

Accordingly, in one embodiment, the process of the present invention for producing proline in a non-human organism, like a microorganism or a plant or a part thereof, comprises increasing or generating the activity of a gene product with the activity of a gene product conferring the activity of “aspartate aminotransferase”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Ykl106w, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Ykl106w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Ykl106w, or a functional equivalent or a homolog thereof as depicted in column 8 of Table II, application no. 2, preferably a homolog or functional equivalent as depicted in column 8 of Table II B, application no. 2, and being depicted in the same respective line as said Ykl106w, and preferably the activity is increased non-targeted.

Accordingly, in one embodiment, the molecule which activity is to be increased in the process of the invention is the gene product with an activity as a “aspartate aminotransferase”, preferably it is the molecule of section (a) or (b) of this paragraph.

In particular, it was observed that in plants, especially in Arabidopsis thaliana, increasing or generating the activity of a gene product non-targeted with the activity of a “aspartate aminotransferase”, preferably being encoded by a gene comprising the nucleic acid sequence SEQ ID NO. 66419, preferably the coding region thereof, conferred the production of or the increase in proline compared with the wild type control.

The nucleic acid sequence of Ylr065c from Saccharomyces cerevisiae, e.g. as shown in the respective line in column 5 of Table I, application no. 2, has been published in Goffeau et al., Science 274 (5287), 546 (1996). And the activity of the gene product thereof is the activity of ylr065c-protein.

Accordingly, in one embodiment, the process of the present invention for producing glutamate in a non-human organism, like a microorganism or a plant or a part thereof, comprises increasing or generating the activity of a gene product with the activity of a gene product conferring the activity of “ylr065c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Ylr065c, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Ylr065c, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Ylr065c, or a functional equivalent or a homolog thereof as depicted in column 8 of Table II, application no. 2, preferably a homolog or functional equivalent as depicted in column 8 of Table II B, application no. 2, and being depicted in the same respective line as said Ylr065c, and preferably the activity is increased plastidic.

Accordingly, in one embodiment, the molecule which activity is to be increased in the process of the invention is the gene product with an activity as a “ylr065c-protein”, preferably it is the molecule of section (a) or (b) of this paragraph.

In particular, it was observed that in plants, especially in Arabidopsis thaliana, increasing or generating the activity of a gene product plastidic with the activity of a “ylr065c-protein”, preferably being encoded by a gene comprising the nucleic acid sequence SEQ ID NO. 66695, preferably the coding region thereof, conferred the production of or the increase in glutamate compared with the wild type control.

The nucleic acid sequence of Ylr178c from Saccharomyces cerevisiae, e.g. as shown in the respective line in column 5 of Table I, application no. 2, has been published in Goffeau et al., Science 274 (5287), 546 (1996). And the activity of the gene product thereof is the activity of ylr178c-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “yid 78c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Ylr178c, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Ylr178c, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Ylr178c, or a functional equivalent or a homolog thereof as depicted in column 8 of Table II, application no. 2, preferably a homolog or functional equivalent as depicted in column 8 of Table II B, application no. 2, and being depicted in the same respective line as said Ylr178c, and preferably the activity is increased plastidic.

Accordingly, in one embodiment, the molecule which activity is to be increased in the process of the invention is the gene product with an activity as a “ylr178c-protein”, preferably it is the molecule of section (a) or (b) of this paragraph.

In particular, it was observed that in plants, especially in Arabidopsis thaliana, increasing or generating the activity of a gene product plastidic with the activity of a “ylr178c-protein”, preferably being encoded by a gene comprising the nucleic acid sequence SEQ ID NO. 66715, preferably the coding region thereof, conferred the production of or the increase in glutamine compared with the wild type control.

The nucleic acid sequence of Ylr304c from Saccharomyces cerevisiae, e.g. as shown in the respective line in column 5 of Table I, application no. 2, has been published in Goffeau et al., Science 274 (5287), 546 (1996). And the activity of the gene product thereof is the activity of aconitate hydratase.

Accordingly, in one embodiment, the process of the present invention for producing proline in a non-human organism, like a microorganism or a plant or a part thereof, comprises increasing or generating the activity of a gene product with the activity of a gene product conferring the activity of “aconitate hydratase”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Ylr304c, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Ylr304c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Ylr304c, or a functional equivalent or a homolog thereof as depicted in column 8 of Table II, application no. 2, preferably a homolog or functional equivalent as depicted in column 8 of Table II B, application no. 2, and being depicted in the same respective line as said Ylr304c, and preferably the activity is increased non-targeted.

Accordingly, in one embodiment, the molecule which activity is to be increased in the process of the invention is the gene product with an activity as a “aconitate hydratase”, preferably it is the molecule of section (a) or (b) of this paragraph.

In particular, it was observed that in plants, especially in Arabidopsis thaliana, increasing or generating the activity of a gene product non-targeted with the activity of a “aconitate hydratase”, preferably being encoded by a gene comprising the nucleic acid sequence SEQ ID NO. 66772, preferably the coding region thereof, conferred the production of or the increase in proline compared with the wild type control.

The nucleic acid sequence of Ylr359w from Saccharomyces cerevisiae, e.g. as shown in the respective line in column 5 of Table I, application no. 2, has been published in Goffeau et al., Science 274 (5287), 546 (1996). And the activity of the gene product thereof is the activity of adenylosuccinate lyase.

Accordingly, in one embodiment, the process of the present invention for producing proline in a non-human organism, like a microorganism or a plant or a part thereof, comprises increasing or generating the activity of a gene product with the activity of a gene product conferring the activity of “adenylosuccinate lyase”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Ylr359w, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Ylr359w, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Ylr359w, or a functional equivalent or a homolog thereof as depicted in column 8 of Table II, application no. 2, preferably a homolog or functional equivalent as depicted in column 8 of Table II B, application no. 2, and being depicted in the same respective line as said Ylr359w, and preferably the activity is increased plastidic,.

Accordingly, in one embodiment, the molecule which activity is to be increased in the process of the invention is the gene product with an activity as a “adenylosuccinate lyase”, preferably it is the molecule of section (a) or (b) of this paragraph.

In particular, it was observed that in plants, especially in Arabidopsis thaliana, increasing or generating the activity of a gene product plastidic with the activity of a “adenylosuccinate lyase”, preferably being encoded by a gene comprising the nucleic acid sequence SEQ ID NO. 67190, preferably the coding region thereof, conferred the production of or the increase in proline compared with the wild type control.

The nucleic acid sequence of Yn1142w from Saccharomyces cerevisiae, e.g. as shown in the respective line in column 5 of Table I, application no. 2, has been published in Goffeau et al., Science 274 (5287), 546 (1996). And the activity of the gene product thereof is the activity of ynl142w-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “ynl142w-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Yn1142w, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Ynl142w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Ynl142w, or a functional equivalent or a homolog thereof as depicted in column 8 of Table II, application no. 2, preferably a homolog or functional equivalent as depicted in column 8 of Table II B, application no. 2, and being depicted in the same respective line as said Ynl142w, and preferably the activity is increased non-targeted.

Accordingly, in one embodiment, the molecule which activity is to be increased in the process of the invention is the gene product with an activity as a “ynl142w-protein”, preferably it is the molecule of section (a) or (b) of this paragraph.

In particular, it was observed that in plants, especially in Arabidopsis thaliana, increasing or generating the activity of a gene product non-targeted with the activity of a “ynl142w-protein”, preferably being encoded by a gene comprising the nucleic acid sequence SEQ ID NO. 67299, preferably the coding region thereof, conferred the production of or the increase in glutamine compared with the wild type control.

The nucleic acid sequence of Ynr019w from Saccharomyces cerevisiae, e.g. as shown in the respective line in column 5 of Table I, application no. 2, has been published in Goffeau et al., Science 274 (5287), 546 (1996). And the activity of the gene product thereof is the activity of sterol O-acyltransferase.

Accordingly, in one embodiment, the process of the present invention for producing proline in a non-human organism, like a microorganism or a plant or a part thereof, comprises increasing or generating the activity of a gene product with the activity of a gene product conferring the activity of “sterol O-acyltransferase”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Ynr019w, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Ynr019w, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Ynr019w, or a functional equivalent or a homolog thereof as depicted in column 8 of Table II, application no. 2, preferably a homolog or functional equivalent as depicted in column 8 of Table II B, application no. 2, and being depicted in the same respective line as said Ynr019w, and preferably the activity is increased plastidic.

Accordingly, in one embodiment, the molecule which activity is to be increased in the process of the invention is the gene product with an activity as a “sterol O-acyltransferase”, preferably it is the molecule of section (a) or (b) of this paragraph.

In particular, it was observed that in plants, especially in Arabidopsis thaliana, increasing or generating the activity of a gene product plastidic with the activity of a “sterol O-acyltransferase”, preferably being encoded by a gene comprising the nucleic acid sequence SEQ ID NO. 67646, preferably the coding region thereof, conferred the production of or the increase in proline compared with the wild type control.

The nucleic acid sequence of Yol045w from Saccharomyces cerevisiae, e.g. as shown in the respective line in column 5 of Table I, application no. 2, has been published in Goffeau et al., Science 274 (5287), 546 (1996). And the activity of the gene product thereof is the activity of protein kinase.

Accordingly, in one embodiment, the process of the present invention for producing arginine in a non-human organism, like a microorganism or a plant or a part thereof, comprises increasing or generating the activity of a gene product with the activity of a gene product conferring the activity of “protein kinase”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Yol045w, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Yol045w, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Yol045w, or a functional equivalent or a homolog thereof as depicted in column 8 of Table II, application no. 2, preferably a homolog or functional equivalent as depicted in column 8 of Table II B, application no. 2, and being depicted in the same respective line as said Yol045w, and preferably the activity is increased plastidic.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product plastidic with the activity of a “protein kinase”, preferably being encoded by a gene comprising the nucleic acid sequence SEQ ID NO. 67684, preferably the coding region thereof, conferred the production of or the increase in arginine compared with the wild type control.

The nucleic acid sequence of Yor168w from Saccharomyces cerevisiae, e.g. as shown in the respective line in column 5 of Table I, application no. 2, has been published in Goffeau et al., Science 274 (5287), 546 (1996). And the activity of the gene product thereof is the activity of glutaminyl-tRNA synthetase.

Accordingly, in one embodiment, the process of the present invention for producing glutamate in a non-human organism, like a microorganism or a plant or a part thereof, comprises increasing or generating the activity of a gene product with the activity of a gene product conferring the activity of “glutaminyl-tRNA synthetase”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Yor168w, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Yor168w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Yor168w, or a functional equivalent or a homolog thereof as depicted in column 8 of Table II, application no. 2, preferably a homolog or functional equivalent as depicted in column 8 of Table II B, application no. 2, and being depicted in the same respective line as said Yor168w, and preferably the activity is increased non-targeted.

Accordingly, in one embodiment, the molecule which activity is to be increased in the process of the invention is the gene product with an activity as a “glutaminyl-tRNA synthetase”, preferably it is the molecule of section (a) or (b) of this paragraph.

In particular, it was observed that in plants, especially in Arabidopsis thaliana, increasing or generating the activity of a gene product non-targeted with the activity of a “glutaminyl-tRNA synthetase”, preferably being encoded by a gene comprising the nucleic acid sequence SEQ ID NO. 67710, preferably the coding region thereof, conferred the production of or the increase in glutamate compared with the wild type control.

The nucleic acid sequence of Yor168w from Saccharomyces cerevisiae, e.g. as shown in the respective line in column 5 of Table I, application no. 2, has been published in Goffeau et al., Science 274 (5287), 546 (1996). And the activity of the gene product thereof is the activity of glutaminyl-tRNA synthetase.

Accordingly, in one embodiment, the process of the present invention for producing glutamine in a non-human organism, like a microorganism or a plant or a part thereof, comprises increasing or generating the activity of a gene product with the activity of a gene product conferring the activity of “glutaminyl-tRNA synthetase”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Yor168w, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Yor168w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Yor168w, or a functional equivalent or a homolog thereof as depicted in column 8 of Table II, application no. 2, preferably a homolog or functional equivalent as depicted in column 8 of Table II B, application no. 2, and being depicted in the same respective line as said Yor168w, and preferably the activity is increased non-targeted.

Accordingly, in one embodiment, the molecule which activity is to be increased in the process of the invention is the gene product with an activity as a “glutaminyl-tRNA synthetase”, preferably it is the molecule of section (a) or (b) of this paragraph.

In particular, it was observed that in plants, especially in Arabidopsis thaliana, increasing or generating the activity of a gene product non-targeted with the activity of a “glutaminyl-tRNA synthetase”, preferably being encoded by a gene comprising the nucleic acid sequence SEQ ID NO. 67710, preferably the coding region thereof, conferred the production of or the increase in glutamine compared with the wild type control.

The nucleic acid sequence of Yor221c from Saccharomyces cerevisiae, e.g. as shown in the respective line in column 5 of Table I, application no. 2, has been published in Goffeau et al., Science 274 (5287), 546 (1996). And the activity of the gene product thereof is the activity of yor221c-protein.

Accordingly, in one embodiment, the process of the present invention for producing proline in a non-human organism, like a microorganism or a plant or a part thereof, comprises increasing or generating the activity of a gene product with the activity of a gene product conferring the activity of “yor221c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Yor221c, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Yor221c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Yor221c, or a functional equivalent or a homolog thereof as depicted in column 8 of Table II, application no. 2, preferably a homolog or functional equivalent as depicted in column 8 of Table II B, application no. 2, and being depicted in the same respective line as said Yor221c, and preferably the activity is increased non-targeted.

Accordingly, in one embodiment, the molecule which activity is to be increased in the process of the invention is the gene product with an activity as a “yor221c-protein”, preferably it is the molecule of section (a) or (b) of this paragraph.

In particular, it was observed that in plants, especially in Arabidopsis thaliana, increasing or generating the activity of a gene product non-targeted with the activity of a “yor221c-protein”, preferably being encoded by a gene comprising the nucleic acid sequence SEQ ID NO. 67951, preferably the coding region thereof, conferred the production of or the increase in proline compared with the wild type control.

The nucleic acid sequence of Yor233w from Saccharomyces cerevisiae, e.g. as shown in the respective line in column 5 of Table I, application no. 2, has been published in Goffeau et al., Science 274 (5287), 546 (1996). And the activity of the gene product thereof is the activity of protein kinase.

Accordingly, in one embodiment, the process of the present invention for producing proline in a non-human organism, like a microorganism or a plant or a part thereof, comprises increasing or generating the activity of a gene product with the activity of a gene product conferring the activity of “protein kinase”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Yor233w, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Yor233w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Yor233w, or a functional equivalent or a homolog thereof as depicted in column 8 of Table II, application no. 2, preferably a homolog or functional equivalent as depicted in column 8 of Table II B, application no. 2, and being depicted in the same respective line as said Yor233w, and preferably the activity is increased non-targeted.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 67968, preferably the coding region thereof, conferred the production of or the increase in proline compared with the wild type control.

The nucleic acid sequence of Ypl117c from Saccharomyces cerevisiae, e.g. as shown in the respective line in column 5 of Table I, application no. 2, has been published in Goffeau et al., Science 274 (5287), 546 (1996). And the activity of the gene product thereof is the activity of isopentenyl diphosphate isomerase.

Accordingly, in one embodiment, the process of the present invention for producing arginine in a non-human organism, like a microorganism or a plant or a part thereof, comprises increasing or generating the activity of a gene product with the activity of a gene product conferring the activity of “isopentenyl diphosphate isomerase”, especially from Saccharomyces cerevisiae or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Ypl117c, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Ypl117c, and preferably the activity is increased plastidic, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Ypl117c, or a functional equivalent or a homolog thereof as depicted in column 8 of Table II, application no. 2, preferably a homolog or functional equivalent as depicted in column 8 of Table II B, application no. 2, and being depicted in the same respective line as said Ypl117c, and preferably the activity is increased plastidic.

Accordingly, in one embodiment, the molecule which activity is to be increased in the process of the invention is the gene product with an activity as a “isopentenyl diphosphate isomerase”, preferably it is the molecule of section (a) or (b) of this paragraph.

In particular, it was observed that in plants, especially in Arabidopsis thaliana, increasing or generating the activity of a gene product plastidic with the activity of a “isopentenyl diphosphate isomerase”, preferably being encoded by a gene comprising the nucleic acid sequence SEQ ID NO. 67998, preferably the coding region thereof, conferred the production of or the increase in arginine compared with the wild type control.

The nucleic acid sequence of Zm_{—}4842_BE510522 from Zea mays, e.g. as shown in the respective line in column 5 of Table I, application no. 2, is unpublished. And the activity of the gene product thereof is the activity of Zm_{—}4842_BE510522-protein.

Accordingly, in one embodiment, the process of the present invention for producing arginine in a non-human organism, like a microorganism or a plant or a part thereof, comprises increasing or generating the activity of a gene product with the activity of a gene product conferring the activity of “Zm_{—}4842_BE510522-protein”, especially from Zea mays or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Zm_{—}4842_BE510522, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Zm_{—}4842_BE510522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Zm_{—}4842_BE510522, or a functional equivalent or a homolog thereof as depicted in column 8 of Table II, application no. 2, preferably a homolog or functional equivalent as depicted in column 8 of Table II B, application no. 2, and being depicted in the same respective line as said Zm_{—}4842_BE510522, and preferably the activity is increased non-targeted.

Accordingly, in one embodiment, the molecule which activity is to be increased in the process of the invention is the gene product with an activity as a “Zm_{—}4842_BE510522-protein”, preferably it is the molecule of section (a) or (b) of this paragraph.

In particular, it was observed that in plants, especially in Arabidopsis thaliana, increasing or generating the activity of a gene product non-targeted with the activity of a “Zm_{—}4842_BE510522-protein”, preferably being encoded by a gene comprising the nucleic acid sequence SEQ ID NO. 68413, preferably the coding region thereof, conferred the production of or the increase in arginine compared with the wild type control.

The nucleic acid sequence of Zm_{—}4842_BE510522 from Zea mays, e.g. as shown in the respective line in column 5 of Table I, application no. 2, is unpublished. And the activity of the gene product thereof is the activity of Zm_{—}4842_BE510522-protein.

Accordingly, in one embodiment, the process of the present invention for producing glutamine in a non-human organism, like a microorganism or a plant or a part thereof, comprises increasing or generating the activity of a gene product with the activity of a gene product conferring the activity of “Zm_{—}4842_BE510522-protein”, especially from Zea mays or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Zm_{—}4842_BE510522, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Zm_{—}4842_BE510522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Zm_{—}4842_BE510522, or a functional equivalent or a homolog thereof as depicted in column 8 of Table II, application no. 2, preferably a homolog or functional equivalent as depicted in column 8 of Table II B, application no. 2, and being depicted in the same respective line as said Zm_{—}4842_BE510522, and preferably the activity is increased non-targeted.

Accordingly, in one embodiment, the molecule which activity is to be increased in the process of the invention is the gene product with an activity as a “Zm_{—}4842_BE510522-protein”, preferably it is the molecule of section (a) or (b) of this paragraph.

In particular, it was observed that in plants, especially in Arabidopsis thaliana, increasing or generating the activity of a gene product non-targeted with the activity of a “Zm_{—}4842_BE510522-protein”, preferably being encoded by a gene comprising the nucleic acid sequence SEQ ID NO. 68413, preferably the coding region thereof, conferred the production of or the increase in glutamine compared with the wild type control.

The nucleic acid sequence of Zm_{—}4842_BE510522 from Zea mays, e.g. as shown in the respective line in column 5 of Table I, application no. 2, is unpublished. And the activity of the gene product thereof is the activity of Zm_{—}4842_BE510522-protein.

Accordingly, in one embodiment, the process of the present invention for producing proline in a non-human organism, like a microorganism or a plant or a part thereof, comprises increasing or generating the activity of a gene product with the activity of a gene product conferring the activity of “Zm_{—}4842_BE510522-protein”, especially from Zea mays or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said Zm_{—}4842_BE510522, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said Zm_{—}4842_BE510522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said Zm_{—}4842_BE510522, or a functional equivalent or a homolog thereof as depicted in column 8 of Table II, application no. 2, preferably a homolog or functional equivalent as depicted in column 8 of Table II B, application no. 2, and being depicted in the same respective line as said Zm_{—}4842_BE510522, and preferably the activity is increased non-targeted.

Accordingly, in one embodiment, the molecule which activity is to be increased in the process of the invention is the gene product with an activity as a “Zm_{—}4842_BE510522-protein”, preferably it is the molecule of section (a) or (b) of this paragraph.

In particular, it was observed that in plants, especially in Arabidopsis thaliana, increasing or generating the activity of a gene product non-targeted with the activity of a “Zm_{—}4842_BE510522-protein”, preferably being encoded by a gene comprising the nucleic acid sequence SEQ ID NO. 68413, preferably the coding region thereof, conferred the production of or the increase in proline compared with the wild type control.

The nucleic acid sequence of ZMO6LC1143 from Zea mays, e.g. as shown in the respective line in column 5 of Table I, application no. 2, is unpublished. And the activity of the gene product thereof is the activity of phosphopantothenoylcysteine decarboxylase.

Accordingly, in one embodiment, the process of the present invention for producing arginine in a non-human organism, like a microorganism or a plant or a part thereof, comprises increasing or generating the activity of a gene product with the activity of a gene product conferring the activity of “phosphopantothenoylcysteine decarboxylase”, especially from Zea mays or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said ZM06LC1143, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said ZMO6LC1143, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said ZM06LC1143, or a functional equivalent or a homolog thereof as depicted in column 8 of Table II, application no. 2, preferably a homolog or functional equivalent as depicted in column 8 of Table II B, application no. 2, and being depicted in the same respective line as said ZM06LC1143, and preferably the activity is increased non-targeted.

Accordingly, in one embodiment, the molecule which activity is to be increased in the process of the invention is the gene product with an activity as a “phosphopantothenoylcysteine decarboxylase”, preferably it is the molecule of section (a) or (b) of this paragraph.

In particular, it was observed that in plants, especially in Arabidopsis thaliana, increasing or generating the activity of a gene product non-targeted with the activity of a “phosphopantothenoylcysteine decarboxylase”, preferably being encoded by a gene comprising the nucleic acid sequence SEQ ID NO. 68132, preferably the coding region thereof, conferred the production of or the increase in arginine compared with the wild type control.

The nucleic acid sequence of ZM06LC11975 from Zea mays, e.g. as shown in the respective line in column 5 of Table I, application no. 2 is unpublished. And the activity of the gene product thereof is the activity of ZM06LC11975-protein.

Accordingly, in one embodiment, the process of the present invention for producing proline in a non-human organism, like a microorganism or a plant or a part thereof, comprises increasing or generating the activity of a gene product with the activity of a gene product conferring the activity of “ZM06LC11975-protein”, especially from Zea mays or its functional equivalent or its homolog, e.g. the increase of
(a) a gene product of a gene comprising the nucleic acid molecule as shown in the respective line in column 5 of Table I, application no. 2, preferably the coding region thereof, or a homolog or a fragment thereof, and being depicted in the same respective line as said ZM06LC11975, or a functional equivalent or a homolog thereof as shown in column 8 of Table I, application no. 2, preferably the coding region thereof, particularly a homolog or functional equivalent as shown in column 8 of Table I B, application no. 2, and being depicted in the same respective line as said ZM06LC11975, and preferably the activity is increased non-targeted, or
(b) a polypeptide comprising a polypeptide, a consensus sequence or at least a polypeptide motif as shown in the respective line in column 5 of Table II or in column 8 of Table IV, application no. 2, respectively, and being depicted in the same respective line as said ZM06LC11975, or a functional equivalent or a homolog thereof as depicted in column 8 of Table II, application no. 2, preferably a homolog or functional equivalent as depicted in column 8 of Table II B, application no. 2, and being depicted in the same respective line as said ZM06LC11975, and preferably the activity is increased non-targeted.

Accordingly, in one embodiment, the molecule which activity is to be increased in the process of the invention is the gene product with an activity as a “ZM06LC11975-protein”, preferably it is the molecule of section (a) or (b) of this paragraph.

In particular, it was observed that in plants, especially in Arabidopsis thaliana, increasing or generating the activity of a gene product non-targeted with the activity of a “ZM06LC11975protein”, preferably being encoded by a gene comprising the nucleic acid sequence SEQ ID NO. 68363, preferably the coding region thereof, conferred the production of or the increase in proline compared with the wild type control.

[0105.1.1.2] to [0107.1.1.2] for the disclosure of these paragraphs see [0105.1.1.1] to [0107.1.1.1] above.

A protein having an activity conferring an increase in the amount or level of the fine chemical arginine, glutamate, glutamine, or proline, upon targeting to the plastids or mitochondria or upon non-targeting, preferably has the structure of the respective polypeptide described herein, in particular of the polypeptides comprising the consensus sequence or at least one polypeptide motifs as shown in the respective line in Table IV, application no. 2, column 8 or of the polypeptide comprising an amino acid sequence as disclosed in the respective line in Table II, application no. 2, columns 5 or 8, or homolos or fragments thereof as described herein, or is encoded by the nucleic acid molecule characterized herein or the nucleic acid molecule according to the invention, for example by the nucleic acid molecule as shown in the respective line in Table I, application no. 2, columns 5 or 8, preferably the coding region thereof, or homologs or fragments thereof and has the herein mentioned activity.

[0109.1.1.2] to [0110.1.1.2] for the disclosure of these paragraphs see [0109.1.1.1] to [0110.1.1.1] above.

In one embodiment, the process of the present invention comprises one or more of the following steps
(a) stabilizing a protein conferring the generation or increased expression of a protein encoded by the nucleic acid molecule of the invention or of the polypeptide of the invention, e.g. of a polypeptide having the activity selected from the group consisting of 2,3-dihydroxy-2,3-dihydrophenylpropionatedehydrogenase, 2-oxoglutarate dehydrogenase E1 subunit, 30S ribosomal protein, 3-deoxy-7-phosphoheptulonate synthase, 3-phosphoglycerate dehydrogenase, 47266012-protein, 49747384_SOYBEAN-protein, 4-alpha-glucanotransferase, ABC transporter permease protein, acetyl CoA carboxylase, acetyltransferase, acid shock protein, aconitate hydratase, acyl-CoA dehydrogenase, acyl-CoA synthase, acyltransferase, adenylosuccinate lyase, adenylylsulfate kinase, alanine dehydrogenase, aldehyde dehydrogenase, allantoicase, allantoinase, amine oxidase, amino acid ABC transporter permease protein, amino acid acetyltransferase, aminotransferase, ankyrin repeat family protein, anthranilate synthase component II, arginine decarboxylase, arginine exporter protein, asparaginase, aspartase, aspartate aminotransferase, At1g17440-protein, At1g19800-protein, At1g29350-protein, At1g47380-protein, Atl g67340-protein, At4g32480-protein, At5g16650-protein, ATP synthase subunit beta, ATP-binding component of a transport system, ATP-dependent RNA helicase, AX653549-protein, AY087308-protein, b0456-protein, b0518-protein, b1003-protein, b1024-protein, b1108-protein, b1137-protein, b1163-protein, b1259-protein, b1280-protein, b1330-protein, b1445-protein, b1522-protein, b1898-protein, b2107-protein, b2121-protein, b2360-protein, b2399-protein, b2474-protein, b2513-protein, b2548-protein, b2613-protein, b2673-protein, b2812-protein, b2846-protein, b2909-protein, b2936-protein, b2999-protein, b3121-protein, b3151-protein, b3346-protein, b3410-protein, B3427-protein, b3509-protein, b3814-protein, b3817-protein, b3989-protein, b4029-protein, b4121-protein, beta-galactosidase, beta-hydroxylase, bifunctional purine biosynthesis protein, branched-chain amino acid ABC transporter permease protein, branched-chain amino acid permease, calcium-dependent protein kinase, carbohydrate kinase, carbon dioxide concentrating mechanism protein, cation/acetate symporter, cation-transporting ATPase, CBL-interacting protein kinase, CCAAT-binding transcription factor, CDP-diacylglycerol-glycerol-3-phosphate 3- phosphatidyltransferase, CDP-diglyceride synthetase, cell division control protein, cell division protein, Chaperone protein CIpB, circadian clock protein, coproporphyrinogen III oxidase, coproporphyrinogen oxidase, CTP synthase, CTP synthetase, cullin, cyclin D, cysteine synthase A, delta-1-pyrroline 5-carboxylase synthetase, dihydrolipoamide acetyltransferase, dihydrolipoamide dehydrogenase, DNA binding protein, DNA helicase II, DNA helicase IV, DNA mismatch repair protein, DnaJ-like chaperone, electron transport complex protein, elongation factor Tu, enoyl-CoA hydratase, enterobacterial common antigen polymerase, ethanolamine utilization protein, eukaryotic translation initiation factor 5, exopolyphosphatase, fatty acid desaturase, flavodoxin, fumarylacetoacetate hydrolase, gamma-glutamyltranspeptidase, geranylgeranyl pyrophosphate synthase, gibberellin 20-oxidase, Glu/Leu/Phe/Val dehydrogenase, gluconate transport system permease 3, glucose dehydrogenase, glucose-1-phosphate cytidylyltransferase, glucose-6-phosphate 1-dehydrogenase, glutamateammonia-ligase, glutamine amidotransferase, glutamine synthetase, glutaminyl-tRNA synthetase, glutaredoxin, glutathione S-transferase, glycerol dehydrogenase, glycerol-3-phosphate dehydrogenase, glycogen (starch) synthase, glycogen synthase, glycogenin, glycoprotease, glycosidase, glycosyl transferase, GM02LC11114-protein, GM02LC17485-protein, GM02LC46-protein, GM02LC5744-protein, H/ACA ribonucleoprotein complex subunit 3, harpin-induced family protein, heat shock protein, heat shock transcription factor, HesB/YadR/YfhF family protein, histone H2A, homocitrate synthase, hydrolase, integral membrane transporter family protein, iron(III) dicitrate-binding protein, isochorismate synthase, isocitrate dehydrogenase, isopentenyl diphosphate isomerase, L-asparaginase, L-aspartate oxidase, Leucyl/phenylalanyl-tRNA-protein transferase, lipoprotein precursor, L-ribulose-5-phosphate 4-epimerase, lysyl-tRNA synthetase, major facilitator superfamily transporter protein, malate dehydrogenase, malic enzyme, malonyl CoA-acyl carrier protein transacylase, membrane protein, membrane transport protein, metal-dependent hydrolase, methylglyoxal synthase, methyltransferase, mitochondrial processing protease, molecular chaperone portein, monothiol glutaredoxin, monthiol glutaredoxin, multiple antibiotic resistance protein, murein transglycosylase, N-acetyl-gamma-glutamyl-phosphate reductase, NAD(P)H-quinone oxidoreductase subunit, NADH dehydrogenase I chain I, nitrate/nitrite transport protein, ornithine carbamoyltransferase, oxidoreductase, oxidoreductase subunit, oxireductase, permease protein of phosphate ABC transporter, peroxisome assembly protein, phosphatidylinositol 3- and 4-kinase family protein, phosphoadenosine phosphosulfate reductase, phosphoanhydride phosphorylase, phosphopantetheine adenylyltransferase, phosphopantothenoylcysteine decarboxylase, phosphopantothenoylcysteinesynthetase/decarboxylase, phosphoribosylaminoimidazole carboxylase catalytic subunit, phosphoribosylformyl glycinamidine synthase subunit, phosphoribosylglycinamide formyltransferase, phosphoribosyltransferase, Photosystem I reaction center subunit XI, photosystem II protein, polygalacturonase, polyphosphate kinase, potassium transport protein, pre-mRNA-splicing factor, protease, protein kinase, protein phosphatase, purine nucleoside phosphorylase, purine-nucleoside phosphorylase, pyrroline carboxylate reductase, pyruvate kinase, quinolinate synthetase, riboflavin biosynthesis protein, ribosephosphate isomerase, constitutive, RNA binding protein, s_pp015018333r-protein, sec-independent protein translocase, Sec-independent protein translocase subunit, serine protease, serine protease inhibitor, short-chain alcohol dehydrogenase family, sll0064-protein, sll0254-protein, sll0354-protein, sll1761-protein, slr0600-protein, sodium/proton antiporter, squalene monooxygenase, sterol O-acyltransferase, thioredoxin, thioredoxin family protein, threonine dehydrogenase, threonine synthase, transcription factor, transcriptional regulator, transcriptional regulator protein, translation initiation factor subunit, transport protein, TTC0768-protein, TTC1386-protein, urease subunit, uridine/cytidine kinase, valine-pyruvate transaminase, XM_{—}473199-protein, YCL026C-A-protein, ycr102c-protein, ydr338c-protein, yer014w-protein, yer106w-protein, YFL019C-protein, yf1054cprotein, yg1237c-protein, ygr068c-protein, ygr221c-protein, yhl013c-protein, yhr207cprotein, ylr065c-protein, ylr178c-protein, ynl142w-protein, yor221c-protein, zinc finger protein, zinc transporter, Zm_{—}4842_BE510522-protein, and ZM06LC11975-protein, or of a polypeptide as indicated in the respective line in Table II, application no. 2, columns 5 or 8, or its homologs or fragments, and conferring the production of or an increase in arginine, glutamate, glutamine, or proline, respectively; and/or
(b) stabilizing a mRNA conferring the generation or increased expression of a FCRP, e.g. protein encoded by the nucleic acid molecule of the invention or its homologs or fragments, or of a mRNA encoding the polypeptide of the present invention having the hereinmentioned activity selected from the group consisting of said activities mentioned in (a) and conferring the production of or an increase in arginine, glutamate, glutamine, or proline, respectively; and/or
(c) increasing the specific activity of a protein conferring the increased expression of a FCRP, e.g. a protein encoded by the nucleic acid molecule of the invention or of the polypeptide of the present invention having herein-mentioned arginine, glutamate, glutamine, or proline generating or increasing activity, respectively, e.g. of a polypeptide having the activity of a protein as indicated in the respective line in Table II, application no. 2, columns 5 or 8, or its homologs or fragments, or decreasing the inhibitiory regulation of the polypeptide of the invention; and/or
(d) generating or increasing the expression of an endogenous or artificial transcription factor mediating the expression of a protein conferring the generation or increased expression of a FCRP, e.g. a protein encoded by the nucleic acid molecule of the invention or of the polypeptide of the invention having herein-mentioned activity selected from the group consisting of said activities mentioned in (a) and conferring a arginine, glutamate, glutamine, or proline increasing activity, respectively, e.g. of a polypeptide having the activity of a protein as indicated in the respective line in Table II, application no. 2, columns 5 or 8 or its homologs; and/or
(e) stimulating activity of a protein conferring the increased expression of a FCRP, e.g. a protein encoded by the nucleic acid molecule of the present invention or a polypeptide of the present invention having herein-mentioned activity selected from the group consisting of said activities mentioned in (a) and conferring a arginine, glutamate, glutamine, or proline increasing activity, respectively, e.g. of a polypeptide having the activity of a protein as indicated in the respective line in Table II, application no. 2, columns 5 or 8, or its homologs or fragments, by adding one or more exogenous inducing factors to the non-human organism or parts thereof; and/or
(f) expressing a transgenic gene encoding a protein conferring the increased expression of a FCRP, e.g. a polypeptide encoded by the nucleic acid molecule of the present invention or a polypeptide of the present invention, having herein-mentioned activity selected from the group consisting of said activities mentioned in (a) and conferring a arginine, glutamate, glutamine, or proline increasing activity, respectively, e.g. of a polypeptide having the activity of a protein as indicated in the respective line in Table II, application no. 2, columns 5 or 8, or its homologs or fragments, and/or
(g) increasing the copy number of a gene conferring the increased expression of a nucleic acid molecule encoding a FCRP, e.g. a polypeptide encoded by the nucleic acid molecule of the invention or the polypeptide of the invention having herein-mentioned activity selected from the group consisting of said activities mentioned in (a) and conferring a arginine, glutamate, glutamine, or proline increasing activity, respectively, e.g. of a polypeptide having the activity of a protein as indicated in the respective line in Table II, application no. 2, columns 5 or 8, or its homologs or fragments; and/or
(h) increasing the expression of the endogenous gene encoding the FCRP, polypeptide of the invention, e.g. a polypeptide encoded by the nucleic acid molecule of the invention or the polypeptide of the invention having herein-mentioned activity selected from the group consisting of said activities mentioned in (a) and conferring a arginine, glutamate, glutamine, or proline; increasing activity, respectively, e.g. of a polypeptide having the activity of a protein as indicated in the respective line in Table II, application no. 2, columns 5 or 8, or its homologs or fragments, by adding positive expression or removing negative expression elements, e.g. homologous recombination can be used to either introduce positive regulatory elements like for plants the 35S enhancer into the promoter or to remove repressor elements form regulatory regions. Further gene conversion methods can be used to disrupt repressor elements or to enhance to acitivty of positive elements. Positive elements can be randomly introduced in plants by T-DNA or transposon mutagenesis and lines can be identified in which the positive elements have be integrated near to a gene of the invention, the expression of which is thereby enhanced; and/or
(i) modulating growth conditions of the non-human organism in such a manner, that the expression or activity of the gene encoding the FCRP, e.g. a protein of the invention or the protein itself is enhanced, for example microorganisms or plants can be grown for example under a higher temperature regime leading to an enhanced expression of heat shock proteins, which can lead to an enhanced arginine, glutamate, glutamine, or proline production, respectively; and/or
(j) selecting of non-human organisms with especially high activity of the proteins of the invention from natural or from mutagenized resources and breeding them into the target organisms, e.g. the elite crops; and/or
(k) directing a protein encoded by the nucleic acid molecule of the invention or of the polypeptide of the present invention having herein-mentioned activity selected from the group consisting of said activities mentioned in (a) and conferring a arginine, glutamate, glutamine, or proline increasing activity, respectively, e.g. of a polypeptide having the activity of a protein as indicated in the respective line in Table II, application no. 2, columns 5 or 8, or its homologs or fragments, if for the corresponding nucleic acid molecule in the respüective line in column 6 of Table I the term “plastidic” is indicated, to the plastids by the addition of a plastidial targeting sequence or if for the corresponding nucleic acid molecule in the respective line in column 6 of Table I the term “mitochondrial” is indicated, to the mitochondria by the addition of a mitochondrial targeting sequence; and/or
(l) generating the expression of a protein encoded by the nucleic acid molecule of the invention or of the polypeptide of the present invention having herein-mentioned activity selected from the group consisting of said activities mentioned in (a) and conferring a arginine, glutamate, glutamine, or proline increasing activity, respectively, e.g. of a polypeptide having the activity of a protein as indicated in the respective line in Table II, application no. 2, columns 5 or 8, or its homologs or fragments, if for the corresponding nucleic acid molecule in the respectzive line in column 6 of Table I the term “plastidic” or “mitochondrial” is indicated, in these organelles by the stable or transient transformation, advantageously stable transformation, of organelles, preferably plastids or mitochondria, with an inventive nucleic acid sequence preferably in form of an expression cassette containing said sequence leading to the expression of the nucleic acids or polypeptides of the invention in the respective organelle; and/or
(m) generating the expression of a protein encoded by the nucleic acid molecule of the invention or of the polypeptide of the present invention having herein-mentioned activity selected from the group consisting of said activities mentioned in (a) and conferring a arginine, glutamate, glutamine, and/or proline increasing activity, respectively, e.g. of a polypeptide having the activity of a protein as indicated in the respective line in Table II, application no. 2, columns 5 or 8, or its homologs or fragments, if for the corresponding nucleic acid molecule in the respective line in column 6 of Table I the term “plastidic” or “mitochondrial” is indicated, in these organelles by integration of a nucleic acid of the invention into the genome of the respective organelle under control of preferable a promoter selective for the respective organelle.

Preferably, said mRNA is the coding region of the nucleic acid molecule of the present invention and/or the protein conferring the increased expression of a protein encoded by the coding region of the nucleic acid molecule of the present invention alone or linked to a transit nucleic acid sequence or transit peptide encoding nucleic acid sequence or the polypeptide having the herein mentioned activity, e.g. conferring the generation of or increase of arginine, glutamate, glutamine, or proline, respectively, after increasing the expression or activity of the encoded polypeptide, non-targeted or in organelles such as plastids and/or mitochondria, preferably plastids, or having the activity of a polypeptide having an activity as the protein as shown in the respective line in Table II, application no. 2, column 3, or its homologs. Preferably the increase of arginine, glutamate, glutamine, or proline, respectively, takes place non-targeted or in plastids and/or mitochondria, preferably non-targeted or in plastids.

[0113.1.1.2] to [0122.1.1.2] for the disclosure of these paragraphs see [0113.1.1.1] to [0122.1.1.1] above.

The activation of an endogenous polypeptide having above-mentioned activity, e.g. having the activity of a protein as shown in the respective line in Table II, application no. 2, column 3 or of the polypeptide of the invention, e.g. conferring the increase of the fine chemical arginine, glutamate, glutamine, or proline, respectively, by increase of expression or activity in the cytoplasm, and/or in the cytosol, and/or in an organelle, such as plastids or mitochondria, can also be increased by introducing a synthetic transcription factor, which binds close to the coding region of the gene encoding the protein as shown in the respective line inTable II, application no. 2, column 5 or 8, or homologs or fragments thereof, and activates its transcription. A chimeric zinc finger protein can be constructed, which comprises a specific DNA-binding domain and an activation domain as e.g. the VP16 domain of Herpes Simplex virus. The specific binding domain can bind to the regulatory region of the gene encoding the protein as shown in the respective line in Table II, application no. 2, column 5 or 8, or homologs or fragments thereof. The expression of the chimeric transcription factor in a non-human organism, in particular in a plant, leads to a specific expression of the protein as shown in the respective line in Table II, application no. 2, column 5 or 8, or homologs or fragments thereof, see e.g. in WO01/52620, Oriz, Proc. Natl. Acad. Sci. USA 99, 13290 (2002), or Guan, Proc. Natl. Acad. Sci. USA 99, 13296 (2002).

[0124.1.1.2] to [0127.1.1.2] for the disclosure of these paragraphs see [0124.1.1.1] to [0127.1.1.1] above.

Owing to the introduction of a gene or a plurality of genes conferring the expression of the nucleic acid molecule of the invention or the polypeptide of the invention, for example the nucleic acid construct mentioned below, or encoding the protein as shown in the respective line in Table II, application no. 2, column 5 or 8, or homologs or fragments thereof, into a non-human organism alone or in combination with other genes, it is possible not only to increase the biosynthetic flux towards the end product, but also to increase, modify or create de novo an advantageous, preferably novel metabolites composition in the non-human organism, e.g. an advantageous composition comprising a higher content of (from a viewpoint of nutrional physiology limited) arginine, glutamate, glutamine, or proline and if desired other amino acids, and/or other metabolites, in free or bound form.

[0129.1.1.2] for the disclosure of this paragraph see [0129.1.1.1] above.

Accordingly, in one embodiment, the process according to the invention relates to a process, which comprises:
(a) providing a non-human organism, preferably a microorganism, a plant cell, a plant tissue, a plant or a part thereof, more preferably a microorganism, a plant tissue, a plant or a part thereof;
(b) increasing the activity of a protein as shown in the respective line in Table II, application no. 2, column 3 or of a polypeptide being encoded by the respective nucleic acid molecule of the present invention and described below, e.g. conferring an increase of the fine chemical arginine, glutamate, glutamine, or proline, respectively, in the non-human organism, preferably in the microorganism, the plant cell, the plant tissue, the plant or a part thereof, more preferably a microorganism, a plant tissue, a plant or a part thereof, especially cytoplasmic or in an organelle, like plastids or mitochondria,
(c) growing the non-human organism, preferably the microorganism, the plant cell, the plant tissue, the plant or a part thereof, under conditions which permit the production of said chemical in the non-human organism, preferably the microorganism, the plant cell, the plant tissue, the plant or a part thereof; and
(d) if desired, recovering, optionally isolating, said fine chemical, in free and/or bound form, and, optionally further free and/or bound amino acids, and/or oher metabolites synthetized by the non-human organism, the microorganism, the plant cell, the plant tissue, the plant or a part thereof.

[0131.1.1.2] to [0139.1.1.2] for the disclosure of these paragraphs see [0131.1.1.1] to [0139.1.1.1] above.

In another embodiment, the invention relates to a (isolated) nucleic acid molecule, which comprises a nucleic acid molecule selected from the group consisting of:
(a) a nucleic acid molecule encoding a polypeptide shown in Table II, application no. 2, column 5 or 8, preferably shown in Table II B, application no. 2, column 8, or a homolog or a fragment thereof;
(b) a nucleic acid molecule shown in Table I, application no. 2, column 5 or 8, preferably shown in Table I B, application no. 2, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 2, preferably in column 8 of Table II B, application no. 2;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 2, preferably shown in column 8 of Table I B, application no. 2, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or
(e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 2;
(i) a nucleic acid molecule encoding a polypeptide having the activity represented by a respective protein as depicted in column 5 of Table II, application no. 2;
(j) a nucleic acid molecule comprising a polynucleotide which is obtained by amplifying a cDNA library or a genomic library using the primers in Table III, application no. 2, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 2.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II application no. 2 and confers a generation or an increase of the respective fine chemical in a non-human organism or a part thereof as compared to a corresponding wild-type non-human organism or part thereof.

In another embodiment, the invention relates to a (isolated) nucleic acid molecule, which comprises a nucleic acid molecule selected from the group consisting of:
(a) a nucleic acid molecule encoding a polypeptide shown in Table II, column 5 or 8, application no. 2, preferably shown in Table II A, application no. 2, in column 5 or in Table II A, application no. 2, column 8 or in Table II B, application no. 2, column 8, or a homolog or a fragment thereof;
(b) a nucleic acid molecule shown in Table I, application no. 2, column 5 or 8, preferably shown in Table I A, application no. 2, in column 5 or in Table I A, application no. 2, column 8 or in Table I B, application no. 2, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 2, preferably shown in Table II A, application no. 2, in column 5 or in Table II A, application no. 2, column 8 or in Table II B, application no. 2, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 2, preferably shown in Table I A, application no. 2, in column 5 or in Table I A, application no. 2, column 8 or in Table I B column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 2;
(i) a nucleic acid molecule encoding a polypeptide having the activity represented by a respective protein as depicted in column 5 of Table II, application no. 2;
(j) a nucleic acid molecule comprising a polynucleotide which is obtained by amplifying a cDNA library or a genomic library using the primers in Table III, application no. 2, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt,500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
   or a nucleic acid molecule comprising a sequence which is complementary thereto;
   whereby, preferably, the nucleic acid molecule according to (a), (b), (c), (d), (e), (f), (g), (h), (i), (j) or (k) distinguishes over the sequence depicted in Table I A and/or I B, application no. 2, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 2, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 2, column 5 or 8, or the coding region thereof, respectively. In a further embodiment the nucleic acid molecule does not encode the polypeptide sequence shown in Table II A and/or II B, application no. 2, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 2, column 5 or 8 and does not encode a protein of the sequence shown in Table II A and/or II B, application no. 2, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 2, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 2, column 5 or 8 but less than 100%, preferably less than 99.999%, 99.99%, 99.9%, 99%, 98%, 97%, 96% or 95% identical to the sequence shown in Table II A and/or II B, application no. 2, column 5 or 8.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 2.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II, application no. 2, and confers a generation or an increase of the respective fine chemical in a non-human organism or a part thereof as compared to a corresponding wild-type non-human organism or part thereof.

[144.1.1.2] to [0155.1.1.2] for the disclosure of these paragraphs see [0144.1.1.1] to [0155.1.1.1] above.

In a preferred embodiment a nucleic acid construct, for example an expression cassette, comprises upstream, i.e. at the 5′ end of the encoding sequence, a promoter and downstream, i.e. at the 3′ end, a polyadenylation signal and optionally other regulatory elements which are operably linked to the intervening encoding sequence with one of the nucleic acids as depicted in the respective line in Table I, application no. 2, column 5 or 8, preferably the coding region thereof, or a homolog or a fragment thereof. By an operable linkage is meant the sequential arrangement of promoter, encoding sequence, terminator, optionally other regulatory elements and optionally targeting sequences in such a way that each of the regulatory elements/targeting sequence can fulfill its function in the expression of the encoding sequence in due manner. In one embodiment the sequences preferred for operable linkage are targeting sequences for ensuring subcellular localization in organelles, like plastids or mitochondria. However, targeting sequences for ensuring subcellular localization in the endoplasmic reticulum (=ER), in the nucleus, in oil corpuscles or other compartments may also be employed as well as translation promoters such as the 5′ lead sequence in tobacco mosaic virus (Gallie et al., Nucl. Acids Res. 15, 8693 (1987)).

for the disclosure of this paragraph see [0157.1.1.1] above.

In an embodiment, the invention relates to an expression cassette, comprising

1) a promoter, prefereably a promoter selected from the group consisting of Big35S, PCUbi, Super and USP;
2) a nucleic acid molecule selected from the group consisting of
(a) a nucleic acid molecule encoding a polypeptide shown in Table II, application no. 2, column 5 or 8, preferably shown in Table II A, application no. 2, column 5, or in Table II A, application no. 2, column 8, or in Table II B, application no. 2, column 8, or a homolog or a fragment thereof;
(b) a nucleic acid molecule shown in Table I, application no. 2, column 5 or 8, preferably shown in Table I A, application no. 2, column 5, or in Table I A, application no. 2, column 8, or in Table I B, application no. 2, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 2, preferably shown in Table II A, application no. 2, column 5, or in Table II A, application no. 2, column 8, or in Table II B, application no. 2, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 2, preferably shown in Table I A, application no. 2, column 5, or in Table I A, application no. 2, column 8,or in Table I B, application no. 2, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 2;
(i) a nucleic acid molecule encoding a polypeptide having the activity represented by a respective protein as depicted in column 5 of Table II, application no. 2;
(j) a nucleic acid molecule comprising a polynucleotide which is obtained by amplifying a cDNA library or a genomic library using the primers in Table III, application no. 2, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
or a nucleic acid molecule comprising a sequence which is complementary thereto.
   and, optionally
   3) nucleic acid molecule encoding a transit peptide, preferably encoding a plastidal transit peptide or a mitochondrial transit peptide;
   which are operable linked.
   (All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 2.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 2 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 2, in the same line as of the respective nucleic acid molecule according to 2) “plastidic” is depicted, or encoding a mitochondrial transit peptide, especially in case in column 6 of Table I, application no. 2, in the same line as of the respective nucleic acid molecule according to 2) “mitochondrial” is depicted.

In another embodiment thereof the expression cassette does not comprise 3) a nucleic acid molecule encoding a transit peptide, especially in case in column 6 of Table I, application no. 2, in the same line as of the respective nucleic acid molecule according to 2) “non-targeted” is depicted.

In a further embodiment, the invention relates to an expression cassette, comprising

1) a promoter, prefereably a promoter selected from the group consisting of Big35S, PCUbi, Super and USP;
2) a nucleic acid molecule selected from the group consisting of
(a) a nucleic acid molecule encoding a polypeptide shown in Table II, application no. 2, column 5 or 8, preferably shown in Table II A, application no. 2, column 5, or in Table II A, application no. 2, column 8, or in Table II B, application no. 2, column 8, or a homolog or a fragment thereof;
(b) a nucleic acid molecule shown in Table I, application no. 2, column 5 or 8, preferably shown in Table I A, application no. 2, column 5, or in Table I A, application no. 2, column 8, or in Table I B, application no. 2, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 2, preferably shown in Table II A, application no. 2, column 5, or in Table II A, application no. 2, column 8, or in Table II B, application no. 2, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 2, preferably shown in Table I A, application no. 2, column 5, or in Table I A, application no. 2, column 8,or in Table I B, application no. 2, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 2;
(i) a nucleic acid molecule encoding a polypeptide having the activity represented by a respective protein as depicted in column 5 of Table II, application no. 2;
(j) a nucleic acid molecule comprising a polynucleotide which is obtained by amplifying a cDNA library or a genomic library using the primers in Table III, application no. 2, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
   and, optionally
   3) nucleic acid molecule encoding a transit peptide, preferably encoding a plastidal transit peptide or a mitochondrial transit peptide;
   which are operable linked and
   whereby, preferably, the nucleic acid molecule according to 2 (a), (b), (c), (d), (e), (f), (g), (h), (i), (j) or (k) distinguishes over the sequence depicted in Table I A and/or I B, application no. 2, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention according to 2) does not consist of the sequence shown in Table I A and/or I B, application no. 2, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 2, column 5 or 8, or the coding region thereof, respectively. In a further embodiment the nucleic acid molecule does not encode the polypeptide sequence shown in Table II A and/or II B, application no. 2, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 2, column 5 or 8 and does not encode a protein of the sequence shown in Table II A and/or II B, application no. 2, column 5 or 8. Accordingly, in one embodiment, the protein encoded by a sequence of a nucleic acid according to 2(a), (b), (c), (d), (e), (f), (g), (h), (i), (j) or (k) does not consist of the sequence shown in Table I A and/or I B, application no. 2, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 2, column 5 or 8 but less than 100%, 99.999%, 99.99%, 99.9%, 99%, 98%, 97%, 96% or 95% identical to the sequence shown in Table II A and/or II B, application no. 2, column 5 or 8.
   (All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 2.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 2 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 2, in the same line as of the respective nucleic acid molecule according to 2) “plastidic” is depicted, or encoding a mitochondrial transit peptide, especially in case in column 6 of Table I, application no. 2, in the same line as of the respective nucleic acid molecule according to 2) “mitochondrial” is depicted.

In another embodiment thereof the expression cassette does not comprise 3) a nucleic acid molecule encoding a transit peptide, especially in case in column 6 of Table I, application no. 2, in the same line as of the respective nucleic acid molecule according to 2) “non-targeted” is depicted.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 36670, or a homolog or fragment thereof, or a homolog thereof being depicted in Table I, application no. 2, column 8, in the same line as SEQ ID NO. 36670, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 36670 is depicted, plastidic is mentioned, or a mitochondrial transit petide in case in Table I, application no. 2, in column 6 in the same line as SEQ ID NO. 36670 is depicted, mitochondrial is mentioned; which is operable linke, too.

In another embodiment thereof the expression cassette does not comprise an additional transit peptide in case in Table I, application no. 2, in column 6 in the same line as SEQ ID NO. 36670 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 35875, or a homolog or fragment thereof, or a homolog thereof being depicted in Table I, application no. 2, column 8, in the same line as SEQ ID NO. 35875, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 35875 is depicted, plastidic is mentioned, or a mitochondrial transit petide in case in Table I, application no. 2, in column 6 in the same line as SEQ ID NO. 35875 is depicted, mitochondrial is mentioned; which is operable linke, too.

In another embodiment thereof the expression cassette does not comprise an additional transit peptide in case in Table I, application no. 2, in column 6 in the same line as SEQ ID NO. 35875 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 7686, or a homolog or fragment thereof, or a homolog thereof being depicted in Table I, application no. 2, column 8, in the same line as SEQ ID NO. 7686, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 7686 is depicted, plastidic is mentioned, or a mitochondrial transit petide in case in Table I, application no. 2, in column 6 in the same line as SEQ ID NO. 7686 is depicted, mitochondrial is mentioned; which is operable linke, too.

In another embodiment thereof the expression cassette does not comprise an additional transit peptide in case in Table I, application no. 2, in column 6 in the same line as SEQ ID NO. 7686 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 35936, or a homolog or fragment thereof, or a homolog thereof being depicted in Table I, application no. 2, column 8, in the same line as SEQ ID NO. 35936, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 35936 is depicted, plastidic is mentioned, or a mitochondrial transit petide in case in Table I, application no. 2, in column 6 in the same line as SEQ ID NO. 35936 is depicted, mitochondrial is mentioned; which is operable linke, too.

In another embodiment thereof the expression cassette does not comprise an additional transit peptide in case in Table I, application no. 2, in column 6 in the same line as SEQ ID NO. 35936 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 35967, or a homolog or fragment thereof, or a homolog thereof being depicted in Table I, application no. 2, column 8, in the same line as SEQ ID NO. 35967, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 35967 is depicted, plastidic is mentioned, or a mitochondrial transit petide in case in Table I, application no. 2, in column 6 in the same line as SEQ ID NO. 35967 is depicted, mitochondrial is mentioned; which is operable linke, too.

In another embodiment thereof the expression cassette does not comprise an additional transit peptide in case in Table I, application no. 2, in column 6 in the same line as SEQ ID NO. 35967 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 36114, or a homolog or fragment thereof, or a homolog thereof being depicted in Table I, application no. 2, column 8, in the same line as SEQ ID NO. 36114, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 36114 is depicted, plastidic is mentioned, or a mitochondrial transit petide in case in Table I, application no. 2, in column 6 in the same line as SEQ ID NO. 36114 is depicted, mitochondrial is mentioned; which is operable linke, too.

In another embodiment thereof the expression cassette does not comprise an additional transit peptide in case in Table I, application no. 2, in column 6 in the same line as SEQ ID NO. 36114 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 36299, or a homolog or fragment thereof, or a homolog thereof being depicted in Table I, application no. 2, column 8, in the same line as SEQ ID NO. 36299, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 36299 is depicted, plastidic is mentioned, or a mitochondrial transit petide in case in Table I, application no. 2, in column 6 in the same line as SEQ ID NO. 36299 is depicted, mitochondrial is mentioned; which is operable linke, too.

In another embodiment thereof the expression cassette does not comprise an additional transit peptide in case in Table I, application no. 2, in column 6 in the same line as SEQ ID NO. 36299 is depicted, non-targeted is mentioned.

In an embodiment the present invention relates to an expresssion cassette comprising a) a promoter, prefereably a promoter selected from the group consisting of Big35S, PCUbi, Super and USP, in particular Superand b) the nucleic acid SEQ ID NO. 36489, or a homolog or fragment thereof, or a homolog thereof being depicted in Table I, application no. 2, column 8, in the same line as SEQ ID NO. 36489, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 36489 is depicted, plastidic is mentioned, or a mitochondrial transit petide in case in Table I, application no. 2, in column 6 in the same line as SEQ ID NO. 36489 is depicted, mitochondrial is mentioned; which is operable linke, too.

In another embodiment thereof the expression cassette does not comprise an additional transit peptide in case in Table I, application no. 2, in column 6 in the same line as SEQ ID NO. 36489 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 9333, or a homolog or fragment thereof, or a homolog thereof being depicted in Table I, application no. 2, column 8, in the same line as SEQ ID NO. 9333, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 9333 is depicted, plastidic is mentioned, or a mitochondrial transit petide in case in Table I, application no. 2, in column 6 in the same line as SEQ ID NO. 9333 is depicted, mitochondrial is mentioned; which is operable linke, too.

In another embodiment thereof the expression cassette does not comprise an additional transit peptide in case in Table I, application no. 2, in column 6 in the same line as SEQ ID NO. 9333 is depicted, non-targeted is mentioned.

In an embodiment the present invention relates to an expresssion cassette comprising a) a promoter, prefereably a promoter selected from the group consisting of Big35S, PCUbi, Super and USP, in particular USP; and b) the nucleic acid SEQ ID NO. 36623, or a homolog or fragment thereof, or a homolog thereof being depicted in Table I, application no. 2, column 8, in the same line as SEQ ID NO. 36623, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 36623 is depicted, plastidic is mentioned, or a mitochondrial transit petide in case in Table I, application no. 2, in column 6 in the same line as SEQ ID NO. 36623 is depicted, mitochondrial is mentioned; which is operable linke, too.

In another embodiment thereof the expression cassette does not comprise an additional transit peptide in case in Table I, application no. 2, in column 6 in the same line as SEQ ID NO. 36623 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 35590, or a homolog or fragment thereof, or a homolog thereof being depicted in Table I, application no. 2, column 8, in the same line as SEQ ID NO. 35590, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 35590 is depicted, plastidic is mentioned, or a mitochondrial transit petide in case in Table I, application no. 2, in column 6 in the same line as SEQ ID NO. 35590 is depicted, mitochondrial is mentioned; which is operable linke, too.

In another embodiment thereof the expression cassette does not comprise an additional transit peptide in case in Table I, application no. 2, in column 6 in the same line as SEQ ID NO. 35590 is depicted, non-targeted is mentioned.

In an embodiment the present invention relates to an expresssion cassette comprising a) a promoter, prefereably a promoter selected from the group consisting of Big35S, PCUbi, Super and USP, in particular USP; and b) the nucleic acid SEQ ID NO. 36809, or a homolog or fragment thereof, or a homolog thereof being depicted in Table I, application no. 2, column 8, in the same line as SEQ ID NO. 36809, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 36809 is depicted, plastidic is mentioned, or a mitochondrial transit petide in case in Table I, application no. 2, in column 6 in the same line as SEQ ID NO. 36809 is depicted, mitochondrial is mentioned; which is operable linke, too.

In another embodiment thereof the expression cassette does not comprise an additional transit peptide in case in Table I, application no. 2, in column 6 in the same line as SEQ ID NO. 36809 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 7941, or a homolog or fragment thereof, or a homolog thereof being depicted in Table I, application no. 2, column 8, in the same line as SEQ ID NO. 7941, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 7941 is depicted, plastidic is mentioned, or a mitochondrial transit petide in case in Table I, application no. 2, in column 6 in the same line as SEQ ID NO. 7941 is depicted, mitochondrial is mentioned; which is operable linke, too.

In another embodiment thereof the expression cassette does not comprise an additional transit peptide in case in Table I, application no. 2, in column 6 in the same line as SEQ ID NO. 7941 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 36880, or a homolog or fragment thereof, or a homolog thereof being depicted in Table I, application no. 2, column 8, in the same line as SEQ ID NO. 36880, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 36880 is depicted, plastidic is mentioned, or a mitochondrial transit petide in case in Table I, application no. 2, in column 6 in the same line as SEQ ID NO. 36880 is depicted, mitochondrial is mentioned; which is operable linke, too.

In another embodiment thereof the expression cassette does not comprise an additional transit peptide in case in Table I, application no. 2, in column 6 in the same line as SEQ ID NO. 36880 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 36907, or a homolog or fragment thereof, or a homolog thereof being depicted in Table I, application no. 2, column 8, in the same line as SEQ ID NO. 36907, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 36907 is depicted, plastidic is mentioned, or a mitochondrial transit petide in case in Table I, application no. 2, in column 6 in the same line as SEQ ID NO. 36907 is depicted, mitochondrial is mentioned; which is operable linke, too.

In another embodiment thereof the expression cassette does not comprise an additional transit peptide in case in Table I, application no. 2, in column 6 in the same line as SEQ ID NO. 36907 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 36937, or a homolog or fragment thereof, or a homolog thereof being depicted in Table I, application no. 2, column 8, in the same line as SEQ ID NO. 36937, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 36937 is depicted, plastidic is mentioned, or a mitochondrial transit petide in case in Table I, application no. 2, in column 6 in the same line as SEQ ID NO. 36937 is depicted, mitochondrial is mentioned; which is operable linke, too.

In another embodiment thereof the expression cassette does not comprise an additional transit peptide in case in Table I, application no. 2, in column 6 in the same line as SEQ ID NO. 36937 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 36971, or a homolog or fragment thereof, or a homolog thereof being depicted in Table I, application no. 2, column 8, in the same line as SEQ ID NO. 36971, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 36971 is depicted, plastidic is mentioned, or a mitochondrial transit petide in case in Table I, application no. 2, in column 6 in the same line as SEQ ID NO. 36971 is depicted, mitochondrial is mentioned; which is operable linke, too.

In another embodiment thereof the expression cassette does not comprise an additional transit peptide in case in Table I, application no. 2, in column 6 in the same line as SEQ ID NO. 36971 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 37390, or a homolog or fragment thereof, or a homolog thereof being depicted in Table I, application no. 2, column 8, in the same line as SEQ ID NO. 37390, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 37390 is depicted, plastidic is mentioned, or a mitochondrial transit petide in case in Table I, application no. 2, in column 6 in the same line as SEQ ID NO. 37390 is depicted, mitochondrial is mentioned; which is operable linke, too.

In another embodiment thereof the expression cassette does not comprise an additional transit peptide in case in Table I, application no. 2, in column 6 in the same line as SEQ ID NO. 37390 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 7917, or a homolog or fragment thereof, or a homolog thereof being depicted in Table I, application no. 2, column 8, in the same line as SEQ ID NO. 7917, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 7917 is depicted, plastidic is mentioned, or a mitochondrial transit petide in case in Table I, application no. 2, in column 6 in the same line as SEQ ID NO. 7917 is depicted, mitochondrial is mentioned; which is operable linke, too.

In another embodiment thereof the expression cassette does not comprise an additional transit peptide in case in Table I, application no. 2, in column 6 in the same line as SEQ ID NO. 7917 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 34204, or a homolog or fragment thereof, or a homolog thereof being depicted in Table I, application no. 2, column 8, in the same line as SEQ ID NO. 34204, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 34204 is depicted, plastidic is mentioned, or a mitochondrial transit petide in case in Table I, application no. 2, in column 6 in the same line as SEQ ID NO. 34204 is depicted, mitochondrial is mentioned; which is operable linke, too.

In another embodiment thereof the expression cassette does not comprise an additional transit peptide in case in Table I, application no. 2, in column 6 in the same line as SEQ ID NO. 34204 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 31926, or a homolog or fragment thereof, or a homolog thereof being depicted in Table I, application no. 2, column 8, in the same line as SEQ ID NO. 31926, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 31926 is depicted, plastidic is mentioned, or a mitochondrial transit petide in case in Table I, application no. 2, in column 6 in the same line as SEQ ID NO. 31926 is depicted, mitochondrial is mentioned; which is operable linke, too.

In another embodiment thereof the expression cassette does not comprise an additional transit peptide in case in Table I, application no. 2, in column 6 in the same line as SEQ ID NO. 31926 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 6510, or a homolog or fragment thereof, or a homolog thereof being depicted in Table I, application no. 2, column 8, in the same line as SEQ ID NO. 6510, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 6510 is depicted, plastidic is mentioned, or a mitochondrial transit petide in case in Table I, application no. 2, in column 6 in the same line as SEQ ID NO. 6510 is depicted, mitochondrial is mentioned; which is operable linke, too.

In another embodiment thereof the expression cassette does not comprise an additional transit peptide in case in Table I, application no. 2, in column 6 in the same line as SEQ ID NO. 6510 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 32037, or a homolog or fragment thereof, or a homolog thereof being depicted in Table I, application no. 2, column 8, in the same line as SEQ ID NO. 32037, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 32037 is depicted, plastidic is mentioned, or a mitochondrial transit petide in case in Table I, application no. 2, in column 6 in the same line as SEQ ID NO. 32037 is depicted, mitochondrial is mentioned; which is operable linke, too.

In another embodiment thereof the expression cassette does not comprise an additional transit peptide in case in Table I, application no. 2, in column 6 in the same line as SEQ ID NO. 32037 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 32308, or a homolog or fragment thereof, or a homolog thereof being depicted in Table I, application no. 2, column 8, in the same line as SEQ ID NO. 32308, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 32308 is depicted, plastidic is mentioned, or a mitochondrial transit petide in case in Table I, application no. 2, in column 6 in the same line as SEQ ID NO. 32308 is depicted, mitochondrial is mentioned; which is operable linke, too.

In another embodiment thereof the expression cassette does not comprise an additional transit peptide in case in Table I, application no. 2, in column 6 in the same line as SEQ ID NO. 32308 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 32648, or a homolog or fragment thereof, or a homolog thereof being depicted in Table I, application no. 2, column 8, in the same line as SEQ ID NO. 32648, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 32648 is depicted, plastidic is mentioned, or a mitochondrial transit petide in case in Table I, application no. 2, in column 6 in the same line as SEQ ID NO. 32648 is depicted, mitochondrial is mentioned; which is operable linke, too.

In another embodiment thereof the expression cassette does not comprise an additional transit peptide in case in Table I, application no. 2, in column 6 in the same line as SEQ ID NO. 32648 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 33085, or a homolog or fragment thereof, or a homolog thereof being depicted in Table I, application no. 2, column 8, in the same line as SEQ ID NO. 33085, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 33085 is depicted, plastidic is mentioned, or a mitochondrial transit petide in case in Table I, application no. 2, in column 6 in the same line as SEQ ID NO. 33085 is depicted, mitochondrial is mentioned; which is operable linke, too.

In another embodiment thereof the expression cassette does not comprise an additional transit peptide in case in Table I, application no. 2, in column 6 in the same line as SEQ ID NO. 33085 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 33457, or a homolog or fragment thereof, or a homolog thereof being depicted in Table I, application no. 2, column 8, in the same line as SEQ ID NO. 33457, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 33457 is depicted, plastidic is mentioned, or a mitochondrial transit petide in case in Table I, application no. 2, in column 6 in the same line as SEQ ID NO. 33457 is depicted, mitochondrial is mentioned; which is operable linke, too.

In another embodiment thereof the expression cassette does not comprise an additional transit peptide in case in Table I, application no. 2, in column 6 in the same line as SEQ ID NO. 33457 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 7333, or a homolog or fragment thereof, or a homolog thereof being depicted in Table I, application no. 2, column 8, in the same line as SEQ ID NO. 7333, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 7333 is depicted, plastidic is mentioned, or a mitochondrial transit petide in case in Table I, application no. 2, in column 6 in the same line as SEQ ID NO. 7333 is depicted, mitochondrial is mentioned; which is operable linke, too.

In another embodiment thereof the expression cassette does not comprise an additional transit peptide in case in Table I, application no. 2, in column 6 in the same line as SEQ ID NO. 7333 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 34044, or a homolog or fragment thereof, or a homolog thereof being depicted in Table I, application no. 2, column 8, in the same line as SEQ ID NO. 34044, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 34044 is depicted, plastidic is mentioned, or a mitochondrial transit petide in case in Table I, application no. 2, in column 6 in the same line as SEQ ID NO. 34044 is depicted, mitochondrial is mentioned; which is operable linke, too.

In another embodiment thereof the expression cassette does not comprise an additional transit peptide in case in Table I, application no. 2, in column 6 in the same line as SEQ ID NO. 34044 is depicted, non-targeted is mentioned.

In an embodiment the present invention relates to an expresssion cassette comprising a) a promoter, prefereably a promoter selected from the group consisting of Big35S, PCUbi, Super and USP, in particular USP; and b) the nucleic acid SEQ ID NO. 35733, or a homolog or fragment thereof, or a homolog thereof being depicted in Table I, application no. 2, column 8, in the same line as SEQ ID NO. 35733, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 35733 is depicted, plastidic is mentioned, or a mitochondrial transit petide in case in Table I, application no. 2, in column 6 in the same line as SEQ ID NO. 35733 is depicted, mitochondrial is mentioned; which is operable linke, too.

In another embodiment thereof the expression cassette does not comprise an additional transit peptide in case in Table I, application no. 2, in column 6 in the same line as SEQ ID NO. 35733 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 34301, or a homolog or fragment thereof, or a homolog thereof being depicted in Table I, application no. 2, column 8, in the same line as SEQ ID NO. 34301, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 34301 is depicted, plastidic is mentioned, or a mitochondrial transit petide in case in Table I, application no. 2, in column 6 in the same line as SEQ ID NO. 34301 is depicted, mitochondrial is mentioned; which is operable linke, too.

In another embodiment thereof the expression cassette does not comprise an additional transit peptide in case in Table I, application no. 2, in column 6 in the same line as SEQ ID NO. 34301 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 34602, or a homolog or fragment thereof, or a homolog thereof being depicted in Table I, application no. 2, column 8, in the same line as SEQ ID NO. 34602, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 34602 is depicted, plastidic is mentioned, or a mitochondrial transit petide in case in Table I, application no. 2, in column 6 in the same line as SEQ ID NO. 34602 is depicted, mitochondrial is mentioned; which is operable linke, too.

In another embodiment thereof the expression cassette does not comprise an additional transit peptide in case in Table I, application no. 2, in column 6 in the same line as SEQ ID NO. 34602 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 34889, or a homolog or fragment thereof, or a homolog thereof being depicted in Table I, application no. 2, column 8, in the same line as SEQ ID NO. 34889, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 34889 is depicted, plastidic is mentioned, or a mitochondrial transit petide in case in Table I, application no. 2, in column 6 in the same line as SEQ ID NO. 34889 is depicted, mitochondrial is mentioned; which is operable linke, too.

In another embodiment thereof the expression cassette does not comprise an additional transit peptide in case in Table I, application no. 2, in column 6 in the same line as SEQ ID NO. 34889 is depicted, non-targeted is mentioned.

In an embodiment the present invention relates to an expresssion cassette comprising a) a promoter, prefereably a promoter selected from the group consisting of Big35S, PCUbi, Super and USP, in particular USP; and b) the nucleic acid SEQ ID NO. 35204, or a homolog or fragment thereof, or a homolog thereof being depicted in Table I, application no. 2, column 8, in the same line as SEQ ID NO. 35204, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 35204 is depicted, plastidic is mentioned, or a mitochondrial transit petide in case in Table I, application no. 2, in column 6 in the same line as SEQ ID NO. 35204 is depicted, mitochondrial is mentioned; which is operable linke, too.

In another embodiment thereof the expression cassette does not comprise an additional transit peptide in case in Table I, application no. 2, in column 6 in the same line as SEQ ID NO. 35204 is depicted, non-targeted is mentioned.

In an embodiment the present invention relates to an expresssion cassette comprising a) a promoter, prefereably a promoter selected from the group consisting of Big35S, PCUbi, Super and USP, in particular USP; and b) the nucleic acid SEQ ID NO. 35366, or a homolog or fragment thereof, or a homolog thereof being depicted in Table I, application no. 2, column 8, in the same line as SEQ ID NO. 35366, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 35366 is depicted, plastidic is mentioned, or a mitochondrial transit petide in case in Table I, application no. 2, in column 6 in the same line as SEQ ID NO. 35366 is depicted, mitochondrial is mentioned; which is operable linke, too.

In another embodiment thereof the expression cassette does not comprise an additional transit peptide in case in Table I, application no. 2, in column 6 in the same line as SEQ ID NO. 35366 is depicted, non-targeted is mentioned.

In an embodiment the present invention relates to an expresssion cassette comprising a) a promoter, prefereably a promoter selected from the group consisting of Big35S, PCUbi, Super and USP, in particular Super and USP; and b) the nucleic acid SEQ ID NO. 35482, or a homolog or fragment thereof, or a homolog thereof being depicted in Table I, application no. 2, column 8, in the same line as SEQ ID NO. 35482, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 35482 is depicted, plastidic is mentioned, or a mitochondrial transit petide in case in Table I, application no. 2, in column 6 in the same line as SEQ ID NO. 35482 is depicted, mitochondrial is mentioned; which is operable linke, too.

In another embodiment thereof the expression cassette does not comprise an additional transit peptide in case in Table I, application no. 2, in column 6 in the same line as SEQ ID NO. 35482 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 7081, or a homolog or fragment thereof, or a homolog thereof being depicted in Table I, application no. 2, column 8, in the same line as SEQ ID NO. 7081, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 7081 is depicted, plastidic is mentioned, or a mitochondrial transit petide in case in Table I, application no. 2, in column 6 in the same line as SEQ ID NO. 7081 is depicted, mitochondrial is mentioned; which is operable linke, too.

In another embodiment thereof the expression cassette does not comprise an additional transit peptide in case in Table I, application no. 2, in column 6 in the same line as SEQ ID NO. 7081 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 37483, or a homolog or fragment thereof, or a homolog thereof being depicted in Table I, application no. 2, column 8, in the same line as SEQ ID NO. 37483, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 37483 is depicted, plastidic is mentioned, or a mitochondrial transit petide in case in Table I, application no. 2, in column 6 in the same line as SEQ ID NO. 37483 is depicted, mitochondrial is mentioned; which is operable linke, too.

In another embodiment thereof the expression cassette does not comprise an additional transit peptide in case in Table I, application no. 2, in column 6 in the same line as SEQ ID NO. 37483 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 33596, or a homolog or fragment thereof, or a homolog thereof being depicted in Table I, application no. 2, column 8, in the same line as SEQ ID NO. 33596, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 33596 is depicted, plastidic is mentioned, or a mitochondrial transit petide in case in Table I, application no. 2, in column 6 in the same line as SEQ ID NO. 33596 is depicted, mitochondrial is mentioned; which is operable linke, too.

In another embodiment thereof the expression cassette does not comprise an additional transit peptide in case in Table I, application no. 2, in column 6 in the same line as SEQ ID NO. 33596 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 40637, or a homolog or fragment thereof, or a homolog thereof being depicted in Table I, application no. 2, column 8, in the same line as SEQ ID NO. 40637, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 40637 is depicted, plastidic is mentioned, or a mitochondrial transit petide in case in Table I, application no. 2, in column 6 in the same line as SEQ ID NO. 40637 is depicted, mitochondrial is mentioned; which is operable linke, too.

In another embodiment thereof the expression cassette does not comprise an additional transit peptide in case in Table I, application no. 2, in column 6 in the same line as SEQ ID NO. 40637 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 37394, or a homolog or fragment thereof, or a homolog thereof being depicted in Table I, application no. 2, column 8, in the same line as SEQ ID NO. 37394, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 37394 is depicted, plastidic is mentioned, or a mitochondrial transit petide in case in Table I, application no. 2, in column 6 in the same line as SEQ ID NO. 37394 is depicted, mitochondrial is mentioned; which is operable linke, too.

In another embodiment thereof the expression cassette does not comprise an additional transit peptide in case in Table I, application no. 2, in column 6 in the same line as SEQ ID NO. 37394 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 39219, or a homolog or fragment thereof, or a homolog thereof being depicted in Table I, application no. 2, column 8, in the same line as SEQ ID NO. 39219, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 39219 is depicted, plastidic is mentioned, or a mitochondrial transit petide in case in Table I, application no. 2, in column 6 in the same line as SEQ ID NO. 39219 is depicted, mitochondrial is mentioned; which is operable linke, too.

In another embodiment thereof the expression cassette does not comprise an additional transit peptide in case in Table I, application no. 2, in column 6 in the same line as SEQ ID NO. 39219 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 39237, or a homolog or fragment thereof, or a homolog thereof being depicted in Table I, application no. 2, column 8, in the same line as SEQ ID NO. 39237, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 39237 is depicted, plastidic is mentioned, or a mitochondrial transit petide in case in Table I, application no. 2, in column 6 in the same line as SEQ ID NO. 39237 is depicted, mitochondrial is mentioned; which is operable linke, too.

In another embodiment thereof the expression cassette does not comprise an additional transit peptide in case in Table I, application no. 2, in column 6 in the same line as SEQ ID NO. 39237 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 39255, or a homolog or fragment thereof, or a homolog thereof being depicted in Table I, application no. 2, column 8, in the same line as SEQ ID NO. 39255, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 39255 is depicted, plastidic is mentioned, or a mitochondrial transit petide in case in Table I, application no. 2, in column 6 in the same line as SEQ ID NO. 39255 is depicted, mitochondrial is mentioned; which is operable linke, too.

In another embodiment thereof the expression cassette does not comprise an additional transit peptide in case in Table I, application no. 2, in column 6 in the same line as SEQ ID NO. 39255 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 39300, or a homolog or fragment thereof, or a homolog thereof being depicted in Table I, application no. 2, column 8, in the same line as SEQ ID NO. 39300, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 39300 is depicted, plastidic is mentioned, or a mitochondrial transit petide in case in Table I, application no. 2, in column 6 in the same line as SEQ ID NO. 39300 is depicted, mitochondrial is mentioned; which is operable linke, too.

In another embodiment thereof the expression cassette does not comprise an additional transit peptide in case in Table I, application no. 2, in column 6 in the same line as SEQ ID NO. 39300 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 40299, or a homolog or fragment thereof, or a homolog thereof being depicted in Table I, application no. 2, column 8, in the same line as SEQ ID NO. 40299, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 40299 is depicted, plastidic is mentioned, or a mitochondrial transit petide in case in Table I, application no. 2, in column 6 in the same line as SEQ ID NO. 40299 is depicted, mitochondrial is mentioned; which is operable linke, too.

In another embodiment thereof the expression cassette does not comprise an additional transit peptide in case in Table I, application no. 2, in column 6 in the same line as SEQ ID NO. 40299 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 40329, or a homolog or fragment thereof, or a homolog thereof being depicted in Table I, application no. 2, column 8, in the same line as SEQ ID NO. 40329, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 40329 is depicted, plastidic is mentioned, or a mitochondrial transit petide in case in Table I, application no. 2, in column 6 in the same line as SEQ ID NO. 40329 is depicted, mitochondrial is mentioned; which is operable linke, too.

In another embodiment thereof the expression cassette does not comprise an additional transit peptide in case in Table I, application no. 2, in column 6 in the same line as SEQ ID NO. 40329 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 39040, or a homolog or fragment thereof, or a homolog thereof being depicted in Table I, application no. 2, column 8, in the same line as SEQ ID NO. 39040, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 39040 is depicted, plastidic is mentioned, or a mitochondrial transit petide in case in Table I, application no. 2, in column 6 in the same line as SEQ ID NO. 39040 is depicted, mitochondrial is mentioned; which is operable linke, too.

In another embodiment thereof the expression cassette does not comprise an additional transit peptide in case in Table I, application no. 2, in column 6 in the same line as SEQ ID NO. 39040 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 40383, or a homolog or fragment thereof, or a homolog thereof being depicted in Table I, application no. 2, column 8, in the same line as SEQ ID NO. 40383, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 40383 is depicted, plastidic is mentioned, or a mitochondrial transit petide in case in Table I, application no. 2, in column 6 in the same line as SEQ ID NO. 40383 is depicted, mitochondrial is mentioned; which is operable linke, too.

In another embodiment thereof the expression cassette does not comprise an additional transit peptide in case in Table I, application no. 2, in column 6 in the same line as SEQ ID NO. 40383 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 39013, or a homolog or fragment thereof, or a homolog thereof being depicted in Table I, application no. 2, column 8, in the same line as SEQ ID NO. 39013, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 39013 is depicted, plastidic is mentioned, or a mitochondrial transit petide in case in Table I, application no. 2, in column 6 in the same line as SEQ ID NO. 39013 is depicted, mitochondrial is mentioned; which is operable linke, too.

In another embodiment thereof the expression cassette does not comprise an additional transit peptide in case in Table I, application no. 2, in column 6 in the same line as SEQ ID NO. 39013 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 40665, or a homolog or fragment thereof, or a homolog thereof being depicted in Table I, application no. 2, column 8, in the same line as SEQ ID NO. 40665, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 40665 is depicted, plastidic is mentioned, or a mitochondrial transit petide in case in Table I, application no. 2, in column 6 in the same line as SEQ ID NO. 40665 is depicted, mitochondrial is mentioned; which is operable linke, too.

In another embodiment thereof the expression cassette does not comprise an additional transit peptide in case in Table I, application no. 2, in column 6 in the same line as SEQ ID NO. 40665 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 40726, or a homolog or fragment thereof, or a homolog thereof being depicted in Table I, application no. 2, column 8, in the same line as SEQ ID NO. 40726, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 40726 is depicted, plastidic is mentioned, or a mitochondrial transit petide in case in Table I, application no. 2, in column 6 in the same line as SEQ ID NO. 40726 is depicted, mitochondrial is mentioned; which is operable linke, too.

In another embodiment thereof the expression cassette does not comprise an additional transit peptide in case in Table I, application no. 2, in column 6 in the same line as SEQ ID NO. 40726 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 9244, or a homolog or fragment thereof, or a homolog thereof being depicted in Table I, application no. 2, column 8, in the same line as SEQ ID NO. 9244, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 9244 is depicted, plastidic is mentioned, or a mitochondrial transit petide in case in Table I, application no. 2, in column 6 in the same line as SEQ ID NO. 9244 is depicted, mitochondrial is mentioned; which is operable linke, too.

In another embodiment thereof the expression cassette does not comprise an additional transit peptide in case in Table I, application no. 2, in column 6 in the same line as SEQ ID NO. 9244 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 40741, or a homolog or fragment thereof, or a homolog thereof being depicted in Table I, application no. 2, column 8, in the same line as SEQ ID NO. 40741, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 40741 is depicted, plastidic is mentioned, or a mitochondrial transit petide in case in Table I, application no. 2, in column 6 in the same line as SEQ ID NO. 40741 is depicted, mitochondrial is mentioned; which is operable linke, too.

In another embodiment thereof the expression cassette does not comprise an additional transit peptide in case in Table I, application no. 2, in column 6 in the same line as SEQ ID NO. 40741 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 40795, or a homolog or fragment thereof, or a homolog thereof being depicted in Table I, application no. 2, column 8, in the same line as SEQ ID NO. 40795, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 40795 is depicted, plastidic is mentioned, or a mitochondrial transit petide in case in Table I, application no. 2, in column 6 in the same line as SEQ ID NO. 40795 is depicted, mitochondrial is mentioned; which is operable linke, too.

In another embodiment thereof the expression cassette does not comprise an additional transit peptide in case in Table I, application no. 2, in column 6 in the same line as SEQ ID NO. 40795 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 40984, or a homolog or fragment thereof, or a homolog thereof being depicted in Table I, application no. 2, column 8, in the same line as SEQ ID NO. 40984, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 40984 is depicted, plastidic is mentioned, or a mitochondrial transit petide in case in Table I, application no. 2, in column 6 in the same line as SEQ ID NO. 40984 is depicted, mitochondrial is mentioned; which is operable linke, too.

In another embodiment thereof the expression cassette does not comprise an additional transit peptide in case in Table I, application no. 2, in column 6 in the same line as SEQ ID NO. 40984 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 41006, or a homolog or fragment thereof, or a homolog thereof being depicted in Table I, application no. 2, column 8, in the same line as SEQ ID NO. 41006, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 41006 is depicted, plastidic is mentioned, or a mitochondrial transit petide in case in Table I, application no. 2, in column 6 in the same line as SEQ ID NO. 41006 is depicted, mitochondrial is mentioned; which is operable linke, too.

In another embodiment thereof the expression cassette does not comprise an additional transit peptide in case in Table I, application no. 2, in column 6 in the same line as SEQ ID NO. 41006 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 15187, or a homolog or fragment thereof, or a homolog thereof being depicted in Table I, application no. 2, column 8, in the same line as SEQ ID NO. 15187, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 15187 is depicted, plastidic is mentioned, or a mitochondrial transit petide in case in Table I, application no. 2, in column 6 in the same line as SEQ ID NO. 15187 is depicted, mitochondrial is mentioned; which is operable linke, too.

In another embodiment thereof the expression cassette does not comprise an additional transit peptide in case in Table I, application no. 2, in column 6 in the same line as SEQ ID NO. 15187 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 9167, or a homolog or fragment thereof, or a homolog thereof being depicted in Table I, application no. 2, column 8, in the same line as SEQ ID NO. 9167, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 9167 is depicted, plastidic is mentioned, or a mitochondrial transit petide in case in Table I, application no. 2, in column 6 in the same line as SEQ ID NO. 9167 is depicted, mitochondrial is mentioned; which is operable linke, too.

In another embodiment thereof the expression cassette does not comprise an additional transit peptide in case in Table I, application no. 2, in column 6 in the same line as SEQ ID NO. 9167 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 7947, or a homolog or fragment thereof, or a homolog thereof being depicted in Table I, application no. 2, column 8, in the same line as SEQ ID NO. 7947, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 7947 is depicted, plastidic is mentioned, or a mitochondrial transit petide in case in Table I, application no. 2, in column 6 in the same line as SEQ ID NO. 7947 is depicted, mitochondrial is mentioned; which is operable linke, too.

In another embodiment thereof the expression cassette does not comprise an additional transit peptide in case in Table I, application no. 2, in column 6 in the same line as SEQ ID NO. 7947 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 30464, or a homolog or fragment thereof, or a homolog thereof being depicted in Table I, application no. 2, column 8, in the same line as SEQ ID NO. 30464, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 30464 is depicted, plastidic is mentioned, or a mitochondrial transit petide in case in Table I, application no. 2, in column 6 in the same line as SEQ ID NO. 30464 is depicted, mitochondrial is mentioned; which is operable linke, too.

In another embodiment thereof the expression cassette does not comprise an additional transit peptide in case in Table I, application no. 2, in column 6 in the same line as SEQ ID NO. 30464 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 37503, or a homolog or fragment thereof, or a homolog thereof being depicted in Table I, application no. 2, column 8, in the same line as SEQ ID NO. 37503, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 37503 is depicted, plastidic is mentioned, or a mitochondrial transit petide in case in Table I, application no. 2, in column 6 in the same line as SEQ ID NO. 37503 is depicted, mitochondrial is mentioned; which is operable linke, too.

In another embodiment thereof the expression cassette does not comprise an additional transit peptide in case in Table I, application no. 2, in column 6 in the same line as SEQ ID NO. 37503 is depicted, non-targeted is mentioned.

In an embodiment the present invention relates to an expresssion cassette comprising a) a promoter, prefereably a promoter selected from the group consisting of Big35S, PCUbi, Super and USP, in particular USP; and b) the nucleic acid SEQ ID NO. 37539, or a homolog or fragment thereof, or a homolog thereof being depicted in Table I, application no. 2, column 8, in the same line as SEQ ID NO. 37539, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 37539 is depicted, plastidic is mentioned, or a mitochondrial transit petide in case in Table I, application no. 2, in column 6 in the same line as SEQ ID NO. 37539 is depicted, mitochondrial is mentioned; which is operable linke, too.

In another embodiment thereof the expression cassette does not comprise an additional transit peptide in case in Table I, application no. 2, in column 6 in the same line as SEQ ID NO. 37539 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 37573, or a homolog or fragment thereof, or a homolog thereof being depicted in Table I, application no. 2, column 8, in the same line as SEQ ID NO. 37573, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 37573 is depicted, plastidic is mentioned, or a mitochondrial transit petide in case in Table I, application no. 2, in column 6 in the same line as SEQ ID NO. 37573 is depicted, mitochondrial is mentioned; which is operable linke, too.

In another embodiment thereof the expression cassette does not comprise an additional transit peptide in case in Table I, application no. 2, in column 6 in the same line as SEQ ID NO. 37573 is depicted, non-targeted is mentioned.

In an embodiment the present invention relates to an expresssion cassette comprising a) a promoter, prefereably a promoter selected from the group consisting of Big35S, PCUbi, Super and USP, in particular USP; and b) the nucleic acid SEQ ID NO. 37658, or a homolog or fragment thereof, or a homolog thereof being depicted in Table I, application no. 2, column 8, in the same line as SEQ ID NO. 37658, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 37658 is depicted, plastidic is mentioned, or a mitochondrial transit petide in case in Table I, application no. 2, in column 6 in the same line as SEQ ID NO. 37658 is depicted, mitochondrial is mentioned; which is operable linke, too.

In another embodiment thereof the expression cassette does not comprise an additional transit peptide in case in Table I, application no. 2, in column 6 in the same line as SEQ ID NO. 37658 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 37807, or a homolog or fragment thereof, or a homolog thereof being depicted in Table I, application no. 2, column 8, in the same line as SEQ ID NO. 37807, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 37807 is depicted, plastidic is mentioned, or a mitochondrial transit petide in case in Table I, application no. 2, in column 6 in the same line as SEQ ID NO. 37807 is depicted, mitochondrial is mentioned; which is operable linke, too.

In another embodiment thereof the expression cassette does not comprise an additional transit peptide in case in Table I, application no. 2, in column 6 in the same line as SEQ ID NO. 37807 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 38226, or a homolog or fragment thereof, or a homolog thereof being depicted in Table I, application no. 2, column 8, in the same line as SEQ ID NO. 38226, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 38226 is depicted, plastidic is mentioned, or a mitochondrial transit petide in case in Table I, application no. 2, in column 6 in the same line as SEQ ID NO. 38226 is depicted, mitochondrial is mentioned; which is operable linke, too.

In another embodiment thereof the expression cassette does not comprise an additional transit peptide in case in Table I, application no. 2, in column 6 in the same line as SEQ ID NO. 38226 is depicted, non-targeted is mentioned.

In an embodiment the present invention relates to an expresssion cassette comprising a) a promoter, prefereably a promoter selected from the group consisting of Big35S, PCUbi, Super and USP, in particular USP; and b) the nucleic acid SEQ ID NO. 39120, or a homolog or fragment thereof, or a homolog thereof being depicted in Table I, application no. 2, column 8, in the same line as SEQ ID NO. 39120, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 39120 is depicted, plastidic is mentioned, or a mitochondrial transit petide in case in Table I, application no. 2, in column 6 in the same line as SEQ ID NO. 39120 is depicted, mitochondrial is mentioned; which is operable linke, too.

In another embodiment thereof the expression cassette does not comprise an additional transit peptide in case in Table I, application no. 2, in column 6 in the same line as SEQ ID NO. 39120 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 38289, or a homolog or fragment thereof, or a homolog thereof being depicted in Table I, application no. 2, column 8, in the same line as SEQ ID NO. 38289, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 38289 is depicted, plastidic is mentioned, or a mitochondrial transit petide in case in Table I, application no. 2, in column 6 in the same line as SEQ ID NO. 38289 is depicted, mitochondrial is mentioned; which is operable linke, too.

In another embodiment thereof the expression cassette does not comprise an additional transit peptide in case in Table I, application no. 2, in column 6 in the same line as SEQ ID NO. 38289 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 37400, or a homolog or fragment thereof, or a homolog thereof being depicted in Table I, application no. 2, column 8, in the same line as SEQ ID NO. 37400, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 37400 is depicted, plastidic is mentioned, or a mitochondrial transit petide in case in Table I, application no. 2, in column 6 in the same line as SEQ ID NO. 37400 is depicted, mitochondrial is mentioned; which is operable linke, too.

In another embodiment thereof the expression cassette does not comprise an additional transit peptide in case in Table I, application no. 2, in column 6 in the same line as SEQ ID NO. 37400 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 38300, or a homolog or fragment thereof, or a homolog thereof being depicted in Table I, application no. 2, column 8, in the same line as SEQ ID NO. 38300, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 38300 is depicted, plastidic is mentioned, or a mitochondrial transit petide in case in Table I, application no. 2, in column 6 in the same line as SEQ ID NO. 38300 is depicted, mitochondrial is mentioned; which is operable linke, too.

In another embodiment thereof the expression cassette does not comprise an additional transit peptide in case in Table I, application no. 2, in column 6 in the same line as SEQ ID NO. 38300 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 38345, or a homolog or fragment thereof, or a homolog thereof being depicted in Table I, application no. 2, column 8, in the same line as SEQ ID NO. 38345, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 38345 is depicted, plastidic is mentioned, or a mitochondrial transit petide in case in Table I, application no. 2, in column 6 in the same line as SEQ ID NO. 38345 is depicted, mitochondrial is mentioned; which is operable linke, too.

In another embodiment thereof the expression cassette does not comprise an additional transit peptide in case in Table I, application no. 2, in column 6 in the same line as SEQ ID NO. 38345 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 38573, or a homolog or fragment thereof, or a homolog thereof being depicted in Table I, application no. 2, column 8, in the same line as SEQ ID NO. 38573, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 38573 is depicted, plastidic is mentioned, or a mitochondrial transit petide in case in Table I, application no. 2, in column 6 in the same line as SEQ ID NO. 38573 is depicted, mitochondrial is mentioned; which is operable linke, too.

In another embodiment thereof the expression cassette does not comprise an additional transit peptide in case in Table I, application no. 2, in column 6 in the same line as SEQ ID NO. 38573 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 38767, or a homolog or fragment thereof, or a homolog thereof being depicted in Table I, application no. 2, column 8, in the same line as SEQ ID NO. 38767, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 38767 is depicted, plastidic is mentioned, or a mitochondrial transit petide in case in Table I, application no. 2, in column 6 in the same line as SEQ ID NO. 38767 is depicted, mitochondrial is mentioned; which is operable linke, too.

In another embodiment thereof the expression cassette does not comprise an additional transit peptide in case in Table I, application no. 2, in column 6 in the same line as SEQ ID NO. 38767 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 38899, or a homolog or fragment thereof, or a homolog thereof being depicted in Table I, application no. 2, column 8, in the same line as SEQ ID NO. 38899, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 38899 is depicted, plastidic is mentioned, or a mitochondrial transit petide in case in Table I, application no. 2, in column 6 in the same line as SEQ ID NO. 38899 is depicted, mitochondrial is mentioned; which is operable linke, too.

In another embodiment thereof the expression cassette does not comprise an additional transit peptide in case in Table I, application no. 2, in column 6 in the same line as SEQ ID NO. 38899 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 38947, or a homolog or fragment thereof, or a homolog thereof being depicted in Table I, application no. 2, column 8, in the same line as SEQ ID NO. 38947, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 38947 is depicted, plastidic is mentioned, or a mitochondrial transit petide in case in Table I, application no. 2, in column 6 in the same line as SEQ ID NO. 38947 is depicted, mitochondrial is mentioned; which is operable linke, too.

In another embodiment thereof the expression cassette does not comprise an additional transit peptide in case in Table I, application no. 2, in column 6 in the same line as SEQ ID NO. 38947 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 8937, or a homolog or fragment thereof, or a homolog thereof being depicted in Table I, application no. 2, column 8, in the same line as SEQ ID NO. 8937, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 8937 is depicted, plastidic is mentioned, or a mitochondrial transit petide in case in Table I, application no. 2, in column 6 in the same line as SEQ ID NO. 8937 is depicted, mitochondrial is mentioned; which is operable linke, too.

In another embodiment thereof the expression cassette does not comprise an additional transit peptide in case in Table I, application no. 2, in column 6 in the same line as SEQ ID NO. 8937 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 39002, or a homolog or fragment thereof, or a homolog thereof being depicted in Table I, application no. 2, column 8, in the same line as SEQ ID NO. 39002, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 39002 is depicted, plastidic is mentioned, or a mitochondrial transit petide in case in Table I, application no. 2, in column 6 in the same line as SEQ ID NO. 39002 is depicted, mitochondrial is mentioned; which is operable linke, too.

In another embodiment thereof the expression cassette does not comprise an additional transit peptide in case in Table I, application no. 2, in column 6 in the same line as SEQ ID NO. 39002 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 38266, or a homolog or fragment thereof, or a homolog thereof being depicted in Table I, application no. 2, column 8, in the same line as SEQ ID NO. 38266, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 38266 is depicted, plastidic is mentioned, or a mitochondrial transit petide in case in Table I, application no. 2, in column 6 in the same line as SEQ ID NO. 38266 is depicted, mitochondrial is mentioned; which is operable linke, too.

In another embodiment thereof the expression cassette does not comprise an additional transit peptide in case in Table I, application no. 2, in column 6 in the same line as SEQ ID NO. 38266 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 21497, or a homolog or fragment thereof, or a homolog thereof being depicted in Table I, application no. 2, column 8, in the same line as SEQ ID NO. 21497, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 21497 is depicted, plastidic is mentioned, or a mitochondrial transit petide in case in Table I, application no. 2, in column 6 in the same line as SEQ ID NO. 21497 is depicted, mitochondrial is mentioned; which is operable linke, too.

In another embodiment thereof the expression cassette does not comprise an additional transit peptide in case in Table I, application no. 2, in column 6 in the same line as SEQ ID NO. 21497 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 23002, or a homolog or fragment thereof, or a homolog thereof being depicted in Table I, application no. 2, column 8, in the same line as SEQ ID NO. 23002, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 23002 is depicted, plastidic is mentioned, or a mitochondrial transit petide in case in Table I, application no. 2, in column 6 in the same line as SEQ ID NO. 23002 is depicted, mitochondrial is mentioned; which is operable linke, too.

In another embodiment thereof the expression cassette does not comprise an additional transit peptide in case in Table I, application no. 2, in column 6 in the same line as SEQ ID NO. 23002 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 19671, or a homolog or fragment thereof, or a homolog thereof being depicted in Table I, application no. 2, column 8, in the same line as SEQ ID NO. 19671, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 19671 is depicted, plastidic is mentioned, or a mitochondrial transit petide in case in Table I, application no. 2, in column 6 in the same line as SEQ ID NO. 19671 is depicted, mitochondrial is mentioned; which is operable linke, too.

In another embodiment thereof the expression cassette does not comprise an additional transit peptide in case in Table I, application no. 2, in column 6 in the same line as SEQ ID NO. 19671 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 19874, or a homolog or fragment thereof, or a homolog thereof being depicted in Table I, application no. 2, column 8, in the same line as SEQ ID NO. 19874, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 19874 is depicted, plastidic is mentioned, or a mitochondrial transit petide in case in Table I, application no. 2, in column 6 in the same line as SEQ ID NO. 19874 is depicted, mitochondrial is mentioned; which is operable linke, too.

In another embodiment thereof the expression cassette does not comprise an additional transit peptide in case in Table I, application no. 2, in column 6 in the same line as SEQ ID NO. 19874 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 19919, or a homolog or fragment thereof, or a homolog thereof being depicted in Table I, application no. 2, column 8, in the same line as SEQ ID NO. 19919, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 19919 is depicted, plastidic is mentioned, or a mitochondrial transit petide in case in Table I, application no. 2, in column 6 in the same line as SEQ ID NO. 19919 is depicted, mitochondrial is mentioned; which is operable linke, too.

In another embodiment thereof the expression cassette does not comprise an additional transit peptide in case in Table I, application no. 2, in column 6 in the same line as SEQ ID NO. 19919 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 20346, or a homolog or fragment thereof, or a homolog thereof being depicted in Table I, application no. 2, column 8, in the same line as SEQ ID NO. 20346, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 20346 is depicted, plastidic is mentioned, or a mitochondrial transit petide in case in Table I, application no. 2, in column 6 in the same line as SEQ ID NO. 20346 is depicted, mitochondrial is mentioned; which is operable linke, too.

In another embodiment thereof the expression cassette does not comprise an additional transit peptide in case in Table I, application no. 2, in column 6 in the same line as SEQ ID NO. 20346 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 20578, or a homolog or fragment thereof, or a homolog thereof being depicted in Table I, application no. 2, column 8, in the same line as SEQ ID NO. 20578, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 20578 is depicted, plastidic is mentioned, or a mitochondrial transit petide in case in Table I, application no. 2, in column 6 in the same line as SEQ ID NO. 20578 is depicted, mitochondrial is mentioned; which is operable linke, too.

In another embodiment thereof the expression cassette does not comprise an additional transit peptide in case in Table I, application no. 2, in column 6 in the same line as SEQ ID NO. 20578 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 21008, or a homolog or fragment thereof, or a homolog thereof being depicted in Table I, application no. 2, column 8, in the same line as SEQ ID NO. 21008, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 21008 is depicted, plastidic is mentioned, or a mitochondrial transit petide in case in Table I, application no. 2, in column 6 in the same line as SEQ ID NO. 21008 is depicted, mitochondrial is mentioned; which is operable linke, too.

In another embodiment thereof the expression cassette does not comprise an additional transit peptide in case in Table I, application no. 2, in column 6 in the same line as SEQ ID NO. 21008 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 19502, or a homolog or fragment thereof, or a homolog thereof being depicted in Table I, application no. 2, column 8, in the same line as SEQ ID NO. 19502, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 19502 is depicted, plastidic is mentioned, or a mitochondrial transit petide in case in Table I, application no. 2, in column 6 in the same line as SEQ ID NO. 19502 is depicted, mitochondrial is mentioned; which is operable linke, too.

In another embodiment thereof the expression cassette does not comprise an additional transit peptide in case in Table I, application no. 2, in column 6 in the same line as SEQ ID NO. 19502 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 21159, or a homolog or fragment thereof, or a homolog thereof being depicted in Table I, application no. 2, column 8, in the same line as SEQ ID NO. 21159, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 21159 is depicted, plastidic is mentioned, or a mitochondrial transit petide in case in Table I, application no. 2, in column 6 in the same line as SEQ ID NO. 21159 is depicted, mitochondrial is mentioned; which is operable linke, too.

In another embodiment thereof the expression cassette does not comprise an additional transit peptide in case in Table I, application no. 2, in column 6 in the same line as SEQ ID NO. 21159 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 19419, or a homolog or fragment thereof, or a homolog thereof being depicted in Table I, application no. 2, column 8, in the same line as SEQ ID NO. 19419, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 19419 is depicted, plastidic is mentioned, or a mitochondrial transit petide in case in Table I, application no. 2, in column 6 in the same line as SEQ ID NO. 19419 is depicted, mitochondrial is mentioned; which is operable linke, too.

In another embodiment thereof the expression cassette does not comprise an additional transit peptide in case in Table I, application no. 2, in column 6 in the same line as SEQ ID NO. 19419 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 21902, or a homolog or fragment thereof, or a homolog thereof being depicted in Table I, application no. 2, column 8, in the same line as SEQ ID NO. 21902, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 21902 is depicted, plastidic is mentioned, or a mitochondrial transit petide in case in Table I, application no. 2, in column 6 in the same line as SEQ ID NO. 21902 is depicted, mitochondrial is mentioned; which is operable linke, too.

In another embodiment thereof the expression cassette does not comprise an additional transit peptide in case in Table I, application no. 2, in column 6 in the same line as SEQ ID NO. 21902 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 22015, or a homolog or fragment thereof, or a homolog thereof being depicted in Table I, application no. 2, column 8, in the same line as SEQ ID NO. 22015, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 22015 is depicted, plastidic is mentioned, or a mitochondrial transit petide in case in Table I, application no. 2, in column 6 in the same line as SEQ ID NO. 22015 is depicted, mitochondrial is mentioned; which is operable linke, too.

In another embodiment thereof the expression cassette does not comprise an additional transit peptide in case in Table I, application no. 2, in column 6 in the same line as SEQ ID NO. 22015 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 22249, or a homolog or fragment thereof, or a homolog thereof being depicted in Table I, application no. 2, column 8, in the same line as SEQ ID NO. 22249, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 22249 is depicted, plastidic is mentioned, or a mitochondrial transit petide in case in Table I, application no. 2, in column 6 in the same line as SEQ ID NO. 22249 is depicted, mitochondrial is mentioned; which is operable linke, too.

In another embodiment thereof the expression cassette does not comprise an additional transit peptide in case in Table I, application no. 2, in column 6 in the same line as SEQ ID NO. 22249 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 22611, or a homolog or fragment thereof, or a homolog thereof being depicted in Table I, application no. 2, column 8, in the same line as SEQ ID NO. 22611, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 22611 is depicted, plastidic is mentioned, or a mitochondrial transit petide in case in Table I, application no. 2, in column 6 in the same line as SEQ ID NO. 22611 is depicted, mitochondrial is mentioned; which is operable linke, too.

In another embodiment thereof the expression cassette does not comprise an additional transit peptide in case in Table I, application no. 2, in column 6 in the same line as SEQ ID NO. 22611 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 22699, or a homolog or fragment thereof, or a homolog thereof being depicted in Table I, application no. 2, column 8, in the same line as SEQ ID NO. 22699, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 22699 is depicted, plastidic is mentioned, or a mitochondrial transit petide in case in Table I, application no. 2, in column 6 in the same line as SEQ ID NO. 22699 is depicted, mitochondrial is mentioned; which is operable linke, too.

In another embodiment thereof the expression cassette does not comprise an additional transit peptide in case in Table I, application no. 2, in column 6 in the same line as SEQ ID NO. 22699 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 22832, or a homolog or fragment thereof, or a homolog thereof being depicted in Table I, application no. 2, column 8, in the same line as SEQ ID NO. 22832, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 22832 is depicted, plastidic is mentioned, or a mitochondrial transit petide in case in Table I, application no. 2, in column 6 in the same line as SEQ ID NO. 22832 is depicted, mitochondrial is mentioned; which is operable linke, too.

In another embodiment thereof the expression cassette does not comprise an additional transit peptide in case in Table I, application no. 2, in column 6 in the same line as SEQ ID NO. 22832 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 22921, or a homolog or fragment thereof, or a homolog thereof being depicted in Table I, application no. 2, column 8, in the same line as SEQ ID NO. 22921, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 22921 is depicted, plastidic is mentioned, or a mitochondrial transit petide in case in Table I, application no. 2, in column 6 in the same line as SEQ ID NO. 22921 is depicted, mitochondrial is mentioned; which is operable linke, too.

In another embodiment thereof the expression cassette does not comprise an additional transit peptide in case in Table I, application no. 2, in column 6 in the same line as SEQ ID NO. 22921 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 31717, or a homolog or fragment thereof, or a homolog thereof being depicted in Table I, application no. 2, column 8, in the same line as SEQ ID NO. 31717, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 31717 is depicted, plastidic is mentioned, or a mitochondrial transit petide in case in Table I, application no. 2, in column 6 in the same line as SEQ ID NO. 31717 is depicted, mitochondrial is mentioned; which is operable linke, too.

In another embodiment thereof the expression cassette does not comprise an additional transit peptide in case in Table I, application no. 2, in column 6 in the same line as SEQ ID NO. 31717 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 21106, or a homolog or fragment thereof, or a homolog thereof being depicted in Table I, application no. 2, column 8, in the same line as SEQ ID NO. 21106, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 21106 is depicted, plastidic is mentioned, or a mitochondrial transit petide in case in Table I, application no. 2, in column 6 in the same line as SEQ ID NO. 21106 is depicted, mitochondrial is mentioned; which is operable linke, too.

In another embodiment thereof the expression cassette does not comprise an additional transit peptide in case in Table I, application no. 2, in column 6 in the same line as SEQ ID NO. 21106 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 17701, or a homolog or fragment thereof, or a homolog thereof being depicted in Table I, application no. 2, column 8, in the same line as SEQ ID NO. 17701, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 17701 is depicted, plastidic is mentioned, or a mitochondrial transit petide in case in Table I, application no. 2, in column 6 in the same line as SEQ ID NO. 17701 is depicted, mitochondrial is mentioned; which is operable linke, too.

In another embodiment thereof the expression cassette does not comprise an additional transit peptide in case in Table I, application no. 2, in column 6 in the same line as SEQ ID NO. 17701 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 69, or a homolog or fragment thereof, or a homolog thereof being depicted in Table I, application no. 2, column 8, in the same line as SEQ ID NO. 69, preferably the coding region thereof, a homolog or a fragment thereof;

which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 69 is depicted, plastidic is mentioned, or a mitochondrial transit petide in case in Table I, application no. 2, in column 6 in the same line as SEQ ID NO. 69 is depicted, mitochondrial is mentioned; which is operable linke, too.

In another embodiment thereof the expression cassette does not comprise an additional transit peptide in case in Table I, application no. 2, in column 6 in the same line as SEQ ID NO. 69 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi,; and b) the nucleic acid SEQ ID NO. 15532, or a homolog or fragment thereof, or a homolog thereof being depicted in Table I, application no. 2, column 8, in the same line as SEQ ID NO. 15532, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 15532 is depicted, plastidic is mentioned, or a mitochondrial transit petide in case in Table I, application no. 2, in column 6 in the same line as SEQ ID NO. 15532 is depicted, mitochondrial is mentioned; which is operable linke, too.

In another embodiment thereof the expression cassette does not comprise an additional transit peptide in case in Table I, application no. 2, in column 6 in the same line as SEQ ID NO. 15532 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 16155, or a homolog or fragment thereof, or a homolog thereof being depicted in Table I, application no. 2, column 8, in the same line as SEQ ID NO. 16155, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 16155 is depicted, plastidic is mentioned, or a mitochondrial transit petide in case in Table I, application no. 2, in column 6 in the same line as SEQ ID NO. 16155 is depicted, mitochondrial is mentioned; which is operable linke, too.

In another embodiment thereof the expression cassette does not comprise an additional transit peptide in case in Table I, application no. 2, in column 6 in the same line as SEQ ID NO. 16155 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 16263, or a homolog or fragment thereof, or a homolog thereof being depicted in Table I, application no. 2, column 8, in the same line as SEQ ID NO. 16263, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 16263 is depicted, plastidic is mentioned, or a mitochondrial transit petide in case in Table I, application no. 2, in column 6 in the same line as SEQ ID NO. 16263 is depicted, mitochondrial is mentioned; which is operable linke, too.

In another embodiment thereof the expression cassette does not comprise an additional transit peptide in case in Table I, application no. 2, in column 6 in the same line as SEQ ID NO. 16263 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 16883, or a homolog or fragment thereof, or a homolog thereof being depicted in Table I, application no. 2, column 8, in the same line as SEQ ID NO. 16883, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 16883 is depicted, plastidic is mentioned, or a mitochondrial transit petide in case in Table I, application no. 2, in column 6 in the same line as SEQ ID NO. 16883 is depicted, mitochondrial is mentioned; which is operable linke, too.

In another embodiment thereof the expression cassette does not comprise an additional transit peptide in case in Table I, application no. 2, in column 6 in the same line as SEQ ID NO. 16883 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 17356, or a homolog or fragment thereof, or a homolog thereof being depicted in Table I, application no. 2, column 8, in the same line as SEQ ID NO. 17356, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 17356 is depicted, plastidic is mentioned, or a mitochondrial transit petide in case in Table I, application no. 2, in column 6 in the same line as SEQ ID NO. 17356 is depicted, mitochondrial is mentioned; which is operable linke, too.

In another embodiment thereof the expression cassette does not comprise an additional transit peptide in case in Table I, application no. 2, in column 6 in the same line as SEQ ID NO. 17356 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 17451, or a homolog or fragment thereof, or a homolog thereof being depicted in Table I, application no. 2, column 8, in the same line as SEQ ID NO. 17451, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 17451 is depicted, plastidic is mentioned, or a mitochondrial transit petide in case in Table I, application no. 2, in column 6 in the same line as SEQ ID NO. 17451 is depicted, mitochondrial is mentioned; which is operable linke, too.

In another embodiment thereof the expression cassette does not comprise an additional transit peptide in case in Table I, application no. 2, in column 6 in the same line as SEQ ID NO. 17451 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 1298, or a homolog or fragment thereof, or a homolog thereof being depicted in Table I, application no. 2, column 8, in the same line as SEQ ID NO. 1298, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 1298 is depicted, plastidic is mentioned, or a mitochondrial transit petide in case in Table I, application no. 2, in column 6 in the same line as SEQ ID NO. 1298 is depicted, mitochondrial is mentioned; which is operable linke, too.

In another embodiment thereof the expression cassette does not comprise an additional transit peptide in case in Table I, application no. 2, in column 6 in the same line as SEQ ID NO. 1298 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 17637, or a homolog or fragment thereof, or a homolog thereof being depicted in Table I, application no. 2, column 8, in the same line as SEQ ID NO. 17637, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 17637 is depicted, plastidic is mentioned, or a mitochondrial transit petide in case in Table I, application no. 2, in column 6 in the same line as SEQ ID NO. 17637 is depicted, mitochondrial is mentioned; which is operable linke, too.

In another embodiment thereof the expression cassette does not comprise an additional transit peptide in case in Table I, application no. 2, in column 6 in the same line as SEQ ID NO. 17637 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 2573, or a homolog or fragment thereof, or a homolog thereof being depicted in Table I, application no. 2, column 8, in the same line as SEQ ID NO. 2573, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 2573 is depicted, plastidic is mentioned, or a mitochondrial transit petide in case in Table I, application no. 2, in column 6 in the same line as SEQ ID NO. 2573 is depicted, mitochondrial is mentioned; which is operable linke, too.

In another embodiment thereof the expression cassette does not comprise an additional transit peptide in case in Table I, application no. 2, in column 6 in the same line as SEQ ID NO. 2573 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 17901, or a homolog or fragment thereof, or a homolog thereof being depicted in Table I, application no. 2, column 8, in the same line as SEQ ID NO. 17901, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 17901 is depicted, plastidic is mentioned, or a mitochondrial transit petide in case in Table I, application no. 2, in column 6 in the same line as SEQ ID NO. 17901 is depicted, mitochondrial is mentioned; which is operable linke, too.

In another embodiment thereof the expression cassette does not comprise an additional transit peptide in case in Table I, application no. 2, in column 6 in the same line as SEQ ID NO. 17901 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 17968, or a homolog or fragment thereof, or a homolog thereof being depicted in Table I, application no. 2, column 8, in the same line as SEQ ID NO. 17968, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 17968 is depicted, plastidic is mentioned, or a mitochondrial transit petide in case in Table I, application no. 2, in column 6 in the same line as SEQ ID NO. 17968 is depicted, mitochondrial is mentioned; which is operable linke, too.

In another embodiment thereof the expression cassette does not comprise an additional transit peptide in case in Table I, application no. 2, in column 6 in the same line as SEQ ID NO. 17968 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 18070, or a homolog or fragment thereof, or a homolog thereof being depicted in Table I, application no. 2, column 8, in the same line as SEQ ID NO. 18070, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 18070 is depicted, plastidic is mentioned, or a mitochondrial transit petide in case in Table I, application no. 2, in column 6 in the same line as SEQ ID NO. 18070 is depicted, mitochondrial is mentioned; which is operable linke, too.

In another embodiment thereof the expression cassette does not comprise an additional transit peptide in case in Table I, application no. 2, in column 6 in the same line as SEQ ID NO. 18070 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 18122, or a homolog or fragment thereof, or a homolog thereof being depicted in Table I, application no. 2, column 8, in the same line as SEQ ID NO. 18122, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 18122 is depicted, plastidic is mentioned, or a mitochondrial transit petide in case in Table I, application no. 2, in column 6 in the same line as SEQ ID NO. 18122 is depicted, mitochondrial is mentioned; which is operable linke, too.

In another embodiment thereof the expression cassette does not comprise an additional transit peptide in case in Table I, application no. 2, in column 6 in the same line as SEQ ID NO. 18122 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 18235, or a homolog or fragment thereof, or a homolog thereof being depicted in Table I, application no. 2, column 8, in the same line as SEQ ID NO. 18235, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 18235 is depicted, plastidic is mentioned, or a mitochondrial transit petide in case in Table I, application no. 2, in column 6 in the same line as SEQ ID NO. 18235 is depicted, mitochondrial is mentioned; which is operable linke, too.

In another embodiment thereof the expression cassette does not comprise an additional transit peptide in case in Table I, application no. 2, in column 6 in the same line as SEQ ID NO. 18235 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 18869, or a homolog or fragment thereof, or a homolog thereof being depicted in Table I, application no. 2, column 8, in the same line as SEQ ID NO. 18869, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 18869 is depicted, plastidic is mentioned, or a mitochondrial transit petide in case in Table I, application no. 2, in column 6 in the same line as SEQ ID NO. 18869 is depicted, mitochondrial is mentioned; which is operable linke, too.

In another embodiment thereof the expression cassette does not comprise an additional transit peptide in case in Table I, application no. 2, in column 6 in the same line as SEQ ID NO. 18869 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 19364, or a homolog or fragment thereof, or a homolog thereof being depicted in Table I, application no. 2, column 8, in the same line as SEQ ID NO. 19364, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 19364 is depicted, plastidic is mentioned, or a mitochondrial transit petide in case in Table I, application no. 2, in column 6 in the same line as SEQ ID NO. 19364 is depicted, mitochondrial is mentioned; which is operable linke, too.

In another embodiment thereof the expression cassette does not comprise an additional transit peptide in case in Table I, application no. 2, in column 6 in the same line as SEQ ID NO. 19364 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 1061, or a homolog or fragment thereof, or a homolog thereof being depicted in Table I, application no. 2, column 8, in the same line as SEQ ID NO. 1061, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 1061 is depicted, plastidic is mentioned, or a mitochondrial transit petide in case in Table I, application no. 2, in column 6 in the same line as SEQ ID NO. 1061 is depicted, mitochondrial is mentioned; which is operable linke, too.

In another embodiment thereof the expression cassette does not comprise an additional transit peptide in case in Table I, application no. 2, in column 6 in the same line as SEQ ID NO. 1061 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 17601, or a homolog or fragment thereof, or a homolog thereof being depicted in Table I, application no. 2, column 8, in the same line as SEQ ID NO. 17601, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 17601 is depicted, plastidic is mentioned, or a mitochondrial transit petide in case in Table I, application no. 2, in column 6 in the same line as SEQ ID NO. 17601 is depicted, mitochondrial is mentioned; which is operable linke, too.

In another embodiment thereof the expression cassette does not comprise an additional transit peptide in case in Table I, application no. 2, in column 6 in the same line as SEQ ID NO. 17601 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 28040, or a homolog or fragment thereof, or a homolog thereof being depicted in Table I, application no. 2, column 8, in the same line as SEQ ID NO. 28040, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 28040 is depicted, plastidic is mentioned, or a mitochondrial transit petide in case in Table I, application no. 2, in column 6 in the same line as SEQ ID NO. 28040 is depicted, mitochondrial is mentioned; which is operable linke, too.

In another embodiment thereof the expression cassette does not comprise an additional transit peptide in case in Table I, application no. 2, in column 6 in the same line as SEQ ID NO. 28040 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 1815, or a homolog or fragment thereof, or a homolog thereof being depicted in Table I, application no. 2, column 8, in the same line as SEQ ID NO. 1815, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 1815 is depicted, plastidic is mentioned, or a mitochondrial transit petide in case in Table I, application no. 2, in column 6 in the same line as SEQ ID NO. 1815 is depicted, mitochondrial is mentioned; which is operable linke, too.

In another embodiment thereof the expression cassette does not comprise an additional transit peptide in case in Table I, application no. 2, in column 6 in the same line as SEQ ID NO. 1815 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 25780, or a homolog or fragment thereof, or a homolog thereof being depicted in Table I, application no. 2, column 8, in the same line as SEQ ID NO. 25780, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 25780 is depicted, plastidic is mentioned, or a mitochondrial transit petide in case in Table I, application no. 2, in column 6 in the same line as SEQ ID NO. 25780 is depicted, mitochondrial is mentioned; which is operable linke, too.

In another embodiment thereof the expression cassette does not comprise an additional transit peptide in case in Table I, application no. 2, in column 6 in the same line as SEQ ID NO. 25780 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 5557, or a homolog or fragment thereof, or a homolog thereof being depicted in Table I, application no. 2, column 8, in the same line as SEQ ID NO. 5557, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 5557 is depicted, plastidic is mentioned, or a mitochondrial transit petide in case in Table I, application no. 2, in column 6 in the same line as SEQ ID NO. 5557 is depicted, mitochondrial is mentioned; which is operable linke, too.

In another embodiment thereof the expression cassette does not comprise an additional transit peptide in case in Table I, application no. 2, in column 6 in the same line as SEQ ID NO. 5557 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 26120, or a homolog or fragment thereof, or a homolog thereof being depicted in Table I, application no. 2, column 8, in the same line as SEQ ID NO. 26120, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 26120 is depicted, plastidic is mentioned, or a mitochondrial transit petide in case in Table I, application no. 2, in column 6 in the same line as SEQ ID NO. 26120 is depicted, mitochondrial is mentioned; which is operable linke, too.

In another embodiment thereof the expression cassette does not comprise an additional transit peptide in case in Table I, application no. 2, in column 6 in the same line as SEQ ID NO. 26120 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 26196, or a homolog or fragment thereof, or a homolog thereof being depicted in Table I, application no. 2, column 8, in the same line as SEQ ID NO. 26196, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 26196 is depicted, plastidic is mentioned, or a mitochondrial transit petide in case in Table I, application no. 2, in column 6 in the same line as SEQ ID NO. 26196 is depicted, mitochondrial is mentioned; which is operable linke, too.

In another embodiment thereof the expression cassette does not comprise an additional transit peptide in case in Table I, application no. 2, in column 6 in the same line as SEQ ID NO. 26196 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 26434, or a homolog or fragment thereof, or a homolog thereof being depicted in Table I, application no. 2, column 8, in the same line as SEQ ID NO. 26434, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 26434 is depicted, plastidic is mentioned, or a mitochondrial transit petide in case in Table I, application no. 2, in column 6 in the same line as SEQ ID NO. 26434 is depicted, mitochondrial is mentioned; which is operable linke, too.

In another embodiment thereof the expression cassette does not comprise an additional transit peptide in case in Table I, application no. 2, in column 6 in the same line as SEQ ID NO. 26434 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbiand b) the nucleic acid SEQ ID NO. 6040, or a homolog or fragment thereof, or a homolog thereof being depicted in Table I, application no. 2, column 8, in the same line as SEQ ID NO. 6040, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 6040 is depicted, plastidic is mentioned, or a mitochondrial transit petide in case in Table I, application no. 2, in column 6 in the same line as SEQ ID NO. 6040 is depicted, mitochondrial is mentioned; which is operable linke, too.

In another embodiment thereof the expression cassette does not comprise an additional transit peptide in case in Table I, application no. 2, in column 6 in the same line as SEQ ID NO. 6040 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 5493, or a homolog or fragment thereof, or a homolog thereof being depicted in Table I, application no. 2, column 8, in the same line as SEQ ID NO. 5493, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 5493 is depicted, plastidic is mentioned, or a mitochondrial transit petide in case in Table I, application no. 2, in column 6 in the same line as SEQ ID NO. 5493 is depicted, mitochondrial is mentioned; which is operable linke, too.

In another embodiment thereof the expression cassette does not comprise an additional transit peptide in case in Table I, application no. 2, in column 6 in the same line as SEQ ID NO. 5493 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 27882, or a homolog or fragment thereof, or a homolog thereof being depicted in Table I, application no. 2, column 8, in the same line as SEQ ID NO. 27882, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 27882 is depicted, plastidic is mentioned, or a mitochondrial transit petide in case in Table I, application no. 2, in column 6 in the same line as SEQ ID NO. 27882 is depicted, mitochondrial is mentioned; which is operable linke, too.

In another embodiment thereof the expression cassette does not comprise an additional transit peptide in case in Table I, application no. 2, in column 6 in the same line as SEQ ID NO. 27882 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 25498, or a homolog or fragment thereof, or a homolog thereof being depicted in Table I, application no. 2, column 8, in the same line as SEQ ID NO. 25498, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 25498 is depicted, plastidic is mentioned, or a mitochondrial transit petide in case in Table I, application no. 2, in column 6 in the same line as SEQ ID NO. 25498 is depicted, mitochondrial is mentioned; which is operable linke, too.

In another embodiment thereof the expression cassette does not comprise an additional transit peptide in case in Table I, application no. 2, in column 6 in the same line as SEQ ID NO. 25498 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 6075, or a homolog or fragment thereof, or a homolog thereof being depicted in Table I, application no. 2, column 8, in the same line as SEQ ID NO. 6075, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 6075 is depicted, plastidic is mentioned, or a mitochondrial transit petide in case in Table I, application no. 2, in column 6 in the same line as SEQ ID NO. 6075 is depicted, mitochondrial is mentioned; which is operable linke, too.

In another embodiment thereof the expression cassette does not comprise an additional transit peptide in case in Table I, application no. 2, in column 6 in the same line as SEQ ID NO. 6075 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 28738, or a homolog or fragment thereof, or a homolog thereof being depicted in Table I, application no. 2, column 8, in the same line as SEQ ID NO. 28738, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 28738 is depicted, plastidic is mentioned, or a mitochondrial transit petide in case in Table I, application no. 2, in column 6 in the same line as SEQ ID NO. 28738 is depicted, mitochondrial is mentioned; which is operable linke, too.

In another embodiment thereof the expression cassette does not comprise an additional transit peptide in case in Table I, application no. 2, in column 6 in the same line as SEQ ID NO. 28738 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 29246, or a homolog or fragment thereof, or a homolog thereof being depicted in Table I, application no. 2, column 8, in the same line as SEQ ID NO. 29246, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 29246 is depicted, plastidic is mentioned, or a mitochondrial transit petide in case in Table I, application no. 2, in column 6 in the same line as SEQ ID NO. 29246 is depicted, mitochondrial is mentioned; which is operable linke, too.

In another embodiment thereof the expression cassette does not comprise an additional transit peptide in case in Table I, application no. 2, in column 6 in the same line as SEQ ID NO. 29246 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 29286, or a homolog or fragment thereof, or a homolog thereof being depicted in Table I, application no. 2, column 8, in the same line as SEQ ID NO. 29286, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 29286 is depicted, plastidic is mentioned, or a mitochondrial transit petide in case in Table I, application no. 2, in column 6 in the same line as SEQ ID NO. 29286 is depicted, mitochondrial is mentioned; which is operable linke, too.

In another embodiment thereof the expression cassette does not comprise an additional transit peptide in case in Table I, application no. 2, in column 6 in the same line as SEQ ID NO. 29286 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 29397, or a homolog or fragment thereof, or a homolog thereof being depicted in Table I, application no. 2, column 8, in the same line as SEQ ID NO. 29397, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 29397 is depicted, plastidic is mentioned, or a mitochondrial transit petide in case in Table I, application no. 2, in column 6 in the same line as SEQ ID NO. 29397 is depicted, mitochondrial is mentioned; which is operable linke, too.

In another embodiment thereof the expression cassette does not comprise an additional transit peptide in case in Table I, application no. 2, in column 6 in the same line as SEQ ID NO. 29397 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 29500, or a homolog or fragment thereof, or a homolog thereof being depicted in Table I, application no. 2, column 8, in the same line as SEQ ID NO. 29500, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 29500 is depicted, plastidic is mentioned, or a mitochondrial transit petide in case in Table I, application no. 2, in column 6 in the same line as SEQ ID NO. 29500 is depicted, mitochondrial is mentioned; which is operable linke, too.

In another embodiment thereof the expression cassette does not comprise an additional transit peptide in case in Table I, application no. 2, in column 6 in the same line as SEQ ID NO. 29500 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 30039, or a homolog or fragment thereof, or a homolog thereof being depicted in Table I, application no. 2, column 8, in the same line as SEQ ID NO. 30039, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 30039 is depicted, plastidic is mentioned, or a mitochondrial transit petide in case in Table I, application no. 2, in column 6 in the same line as SEQ ID NO. 30039 is depicted, mitochondrial is mentioned; which is operable linke, too.

In another embodiment thereof the expression cassette does not comprise an additional transit peptide in case in Table I, application no. 2, in column 6 in the same line as SEQ ID NO. 30039 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 41442, or a homolog or fragment thereof, or a homolog thereof being depicted in Table I, application no. 2, column 8, in the same line as SEQ ID NO. 41442, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 41442 is depicted, plastidic is mentioned, or a mitochondrial transit petide in case in Table I, application no. 2, in column 6 in the same line as SEQ ID NO. 41442 is depicted, mitochondrial is mentioned; which is operable linke, too.

In another embodiment thereof the expression cassette does not comprise an additional transit peptide in case in Table I, application no. 2, in column 6 in the same line as SEQ ID NO. 41442 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 27021, or a homolog or fragment thereof, or a homolog thereof being depicted in Table I, application no. 2, column 8, in the same line as SEQ ID NO. 27021, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 27021 is depicted, plastidic is mentioned, or a mitochondrial transit petide in case in Table I, application no. 2, in column 6 in the same line as SEQ ID NO. 27021 is depicted, mitochondrial is mentioned; which is operable linke, too.

In another embodiment thereof the expression cassette does not comprise an additional transit peptide in case in Table I, application no. 2, in column 6 in the same line as SEQ ID NO. 27021 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 4348, or a homolog or fragment thereof, or a homolog thereof being depicted in Table I, application no. 2, column 8, in the same line as SEQ ID NO. 4348, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 4348 is depicted, plastidic is mentioned, or a mitochondrial transit petide in case in Table I, application no. 2, in column 6 in the same line as SEQ ID NO. 4348 is depicted, mitochondrial is mentioned; which is operable linke, too.

In another embodiment thereof the expression cassette does not comprise an additional transit peptide in case in Table I, application no. 2, in column 6 in the same line as SEQ ID NO. 4348 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 2935, or a homolog or fragment thereof, or a homolog thereof being depicted in Table I, application no. 2, column 8, in the same line as SEQ ID NO. 2935, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 2935 is depicted, plastidic is mentioned, or a mitochondrial transit petide in case in Table I, application no. 2, in column 6 in the same line as SEQ ID NO. 2935 is depicted, mitochondrial is mentioned; which is operable linke, too.

In another embodiment thereof the expression cassette does not comprise an additional transit peptide in case in Table I, application no. 2, in column 6 in the same line as SEQ ID NO. 2935 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 23482, or a homolog or fragment thereof, or a homolog thereof being depicted in Table I, application no. 2, column 8, in the same line as SEQ ID NO. 23482, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 23482 is depicted, plastidic is mentioned, or a mitochondrial transit petide in case in Table I, application no. 2, in column 6 in the same line as SEQ ID NO. 23482 is depicted, mitochondrial is mentioned; which is operable linke, too.

In another embodiment thereof the expression cassette does not comprise an additional transit peptide in case in Table I, application no. 2, in column 6 in the same line as SEQ ID NO. 23482 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 3279, or a homolog or fragment thereof, or a homolog thereof being depicted in Table I, application no. 2, column 8, in the same line as SEQ ID NO. 3279, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 3279 is depicted, plastidic is mentioned, or a mitochondrial transit petide in case in Table I, application no. 2, in column 6 in the same line as SEQ ID NO. 3279 is depicted, mitochondrial is mentioned; which is operable linke, too.

In another embodiment thereof the expression cassette does not comprise an additional transit peptide in case in Table I, application no. 2, in column 6 in the same line as SEQ ID NO. 3279 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 23844, or a homolog or fragment thereof, or a homolog thereof being depicted in Table I, application no. 2, column 8, in the same line as SEQ ID NO. 23844, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 23844 is depicted, plastidic is mentioned, or a mitochondrial transit petide in case in Table I, application no. 2, in column 6 in the same line as SEQ ID NO. 23844 is depicted, mitochondrial is mentioned; which is operable linke, too.

In another embodiment thereof the expression cassette does not comprise an additional transit peptide in case in Table I, application no. 2, in column 6 in the same line as SEQ ID NO. 23844 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 3654, or a homolog or fragment thereof, or a homolog thereof being depicted in Table I, application no. 2, column 8, in the same line as SEQ ID NO. 3654, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 3654 is depicted, plastidic is mentioned, or a mitochondrial transit petide in case in Table I, application no. 2, in column 6 in the same line as SEQ ID NO. 3654 is depicted, mitochondrial is mentioned; which is operable linke, too.

In another embodiment thereof the expression cassette does not comprise an additional transit peptide in case in Table I, application no. 2, in column 6 in the same line as SEQ ID NO. 3654 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 24232, or a homolog or fragment thereof, or a homolog thereof being depicted in Table I, application no. 2, column 8, in the same line as SEQ ID NO. 24232, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 24232 is depicted, plastidic is mentioned, or a mitochondrial transit petide in case in Table I, application no. 2, in column 6 in the same line as SEQ ID NO. 24232 is depicted, mitochondrial is mentioned; which is operable linke, too.

In another embodiment thereof the expression cassette does not comprise an additional transit peptide in case in Table I, application no. 2, in column 6 in the same line as SEQ ID NO. 24232 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 4040, or a homolog or fragment thereof, or a homolog thereof being depicted in Table I, application no. 2, column 8, in the same line as SEQ ID NO. 4040, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 4040 is depicted, plastidic is mentioned, or a mitochondrial transit petide in case in Table I, application no. 2, in column 6 in the same line as SEQ ID NO. 4040 is depicted, mitochondrial is mentioned; which is operable linke, too.

In another embodiment thereof the expression cassette does not comprise an additional transit peptide in case in Table I, application no. 2, in column 6 in the same line as SEQ ID NO. 4040 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 25676, or a homolog or fragment thereof, or a homolog thereof being depicted in Table I, application no. 2, column 8, in the same line as SEQ ID NO. 25676, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 25676 is depicted, plastidic is mentioned, or a mitochondrial transit petide in case in Table I, application no. 2, in column 6 in the same line as SEQ ID NO. 25676 is depicted, mitochondrial is mentioned; which is operable linke, too.

In another embodiment thereof the expression cassette does not comprise an additional transit peptide in case in Table I, application no. 2, in column 6 in the same line as SEQ ID NO. 25676 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 24311, or a homolog or fragment thereof, or a homolog thereof being depicted in Table I, application no. 2, column 8, in the same line as SEQ ID NO. 24311, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 24311 is depicted, plastidic is mentioned, or a mitochondrial transit petide in case in Table I, application no. 2, in column 6 in the same line as SEQ ID NO. 24311 is depicted, mitochondrial is mentioned; which is operable linke, too.

In another embodiment thereof the expression cassette does not comprise an additional transit peptide in case in Table I, application no. 2, in column 6 in the same line as SEQ ID NO. 24311 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 31026, or a homolog or fragment thereof, or a homolog thereof being depicted in Table I, application no. 2, column 8, in the same line as SEQ ID NO. 31026, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 31026 is depicted, plastidic is mentioned, or a mitochondrial transit petide in case in Table I, application no. 2, in column 6 in the same line as SEQ ID NO. 31026 is depicted, mitochondrial is mentioned; which is operable linke, too.

In another embodiment thereof the expression cassette does not comprise an additional transit peptide in case in Table I, application no. 2, in column 6 in the same line as SEQ ID NO. 31026 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 24438, or a homolog or fragment thereof, or a homolog thereof being depicted in Table I, application no. 2, column 8, in the same line as SEQ ID NO. 24438, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 24438 is depicted, plastidic is mentioned, or a mitochondrial transit petide in case in Table I, application no. 2, in column 6 in the same line as SEQ ID NO. 24438 is depicted, mitochondrial is mentioned; which is operable linke, too.

In another embodiment thereof the expression cassette does not comprise an additional transit peptide in case in Table I, application no. 2, in column 6 in the same line as SEQ ID NO. 24438 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 24492, or a homolog or fragment thereof, or a homolog thereof being depicted in Table I, application no. 2, column 8, in the same line as SEQ ID NO. 24492, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 24492 is depicted, plastidic is mentioned, or a mitochondrial transit petide in case in Table I, application no. 2, in column 6 in the same line as SEQ ID NO. 24492 is depicted, mitochondrial is mentioned; which is operable linke, too.

In another embodiment thereof the expression cassette does not comprise an additional transit peptide in case in Table I, application no. 2, in column 6 in the same line as SEQ ID NO. 24492 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 25222, or a homolog or fragment thereof, or a homolog thereof being depicted in Table I, application no. 2, column 8, in the same line as SEQ ID NO. 25222, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 25222 is depicted, plastidic is mentioned, or a mitochondrial transit petide in case in Table I, application no. 2, in column 6 in the same line as SEQ ID NO. 25222 is depicted, mitochondrial is mentioned; which is operable linke, too.

In another embodiment thereof the expression cassette does not comprise an additional transit peptide in case in Table I, application no. 2, in column 6 in the same line as SEQ ID NO. 25222 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 25283, or a homolog or fragment thereof, or a homolog thereof being depicted in Table I, application no. 2, column 8, in the same line as SEQ ID NO. 25283, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 25283 is depicted, plastidic is mentioned, or a mitochondrial transit petide in case in Table I, application no. 2, in column 6 in the same line as SEQ ID NO. 25283 is depicted, mitochondrial is mentioned; which is operable linke, too.

In another embodiment thereof the expression cassette does not comprise an additional transit peptide in case in Table I, application no. 2, in column 6 in the same line as SEQ ID NO. 25283 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 4904, or a homolog or fragment thereof, or a homolog thereof being depicted in Table I, application no. 2, column 8, in the same line as SEQ ID NO. 4904, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 4904 is depicted, plastidic is mentioned, or a mitochondrial transit petide in case in Table I, application no. 2, in column 6 in the same line as

SEQ ID NO. 4904 is depicted, mitochondrial is mentioned; which is operable linke, too.

In another embodiment thereof the expression cassette does not comprise an additional transit peptide in case in Table I, application no. 2, in column 6 in the same line as SEQ ID NO. 4904 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 25344, or a homolog or fragment thereof, or a homolog thereof being depicted in Table I, application no. 2, column 8, in the same line as SEQ ID NO. 25344, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 25344 is depicted, plastidic is mentioned, or a mitochondrial transit petide in case in Table I, application no. 2, in column 6 in the same line as SEQ ID NO. 25344 is depicted, mitochondrial is mentioned; which is operable linke, too.

In another embodiment thereof the expression cassette does not comprise an additional transit peptide in case in Table I, application no. 2, in column 6 in the same line as SEQ ID NO. 25344 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 25428, or a homolog or fragment thereof, or a homolog thereof being depicted in Table I, application no. 2, column 8, in the same line as SEQ ID NO. 25428, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 25428 is depicted, plastidic is mentioned, or a mitochondrial transit petide in case in Table I, application no. 2, in column 6 in the same line as SEQ ID NO. 25428 is depicted, mitochondrial is mentioned; which is operable linke, too.

In another embodiment thereof the expression cassette does not comprise an additional transit peptide in case in Table I, application no. 2, in column 6 in the same line as SEQ ID NO. 25428 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 5318, or a homolog or fragment thereof, or a homolog thereof being depicted in Table I, application no. 2, column 8, in the same line as SEQ ID NO. 5318, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 5318 is depicted, plastidic is mentioned, or a mitochondrial transit petide in case in Table I, application no. 2, in column 6 in the same line as SEQ ID NO. 5318 is depicted, mitochondrial is mentioned; which is operable linke, too.

In another embodiment thereof the expression cassette does not comprise an additional transit peptide in case in Table I, application no. 2, in column 6 in the same line as SEQ ID NO. 5318 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 4102, or a homolog or fragment thereof, or a homolog thereof being depicted in Table I, application no. 2, column 8, in the same line as SEQ ID NO. 4102, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 4102 is depicted, plastidic is mentioned, or a mitochondrial transit petide in case in Table I, application no. 2, in column 6 in the same line as SEQ ID NO. 4102 is depicted, mitochondrial is mentioned; which is operable linke, too.

In another embodiment thereof the expression cassette does not comprise an additional transit peptide in case in Table I, application no. 2, in column 6 in the same line as SEQ ID NO. 4102 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 62244, or a homolog or fragment thereof, or a homolog thereof being depicted in Table I, application no. 2, column 8, in the same line as SEQ ID NO. 62244, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 62244 is depicted, plastidic is mentioned, or a mitochondrial transit petide in case in Table I, application no. 2, in column 6 in the same line as SEQ ID NO. 62244 is depicted, mitochondrial is mentioned; which is operable linke, too.

In another embodiment thereof the expression cassette does not comprise an additional transit peptide in case in Table I, application no. 2, in column 6 in the same line as SEQ ID NO. 62244 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 60301, or a homolog or fragment thereof, or a homolog thereof being depicted in Table I, application no. 2, column 8, in the same line as SEQ ID NO. 60301, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 60301 is depicted, plastidic is mentioned, or a mitochondrial transit petide in case in Table I, application no. 2, in column 6 in the same line as SEQ ID NO. 60301 is depicted, mitochondrial is mentioned; which is operable linke, too.

In another embodiment thereof the expression cassette does not comprise an additional transit peptide in case in Table I, application no. 2, in column 6 in the same line as SEQ ID NO. 60301 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 60859, or a homolog or fragment thereof, or a homolog thereof being depicted in Table I, application no. 2, column 8, in the same line as SEQ ID NO. 60859, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 60859 is depicted, plastidic is mentioned, or a mitochondrial transit petide in case in Table I, application no. 2, in column 6 in the same line as SEQ ID NO. 60859 is depicted, mitochondrial is mentioned; which is operable linke, too.

In another embodiment thereof the expression cassette does not comprise an additional transit peptide in case in Table I, application no. 2, in column 6 in the same line as SEQ ID NO. 60859 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 61070, or a homolog or fragment thereof, or a homolog thereof being depicted in Table I, application no. 2, column 8, in the same line as SEQ ID NO. 61070, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 61070 is depicted, plastidic is mentioned, or a mitochondrial transit petide in case in Table I, application no. 2, in column 6 in the same line as SEQ ID NO. 61070 is depicted, mitochondrial is mentioned; which is operable linke, too.

In another embodiment thereof the expression cassette does not comprise an additional transit peptide in case in Table I, application no. 2, in column 6 in the same line as SEQ ID NO. 61070 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 61532, or a homolog or fragment thereof, or a homolog thereof being depicted in Table I, application no. 2, column 8, in the same line as SEQ ID NO. 61532, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 61532 is depicted, plastidic is mentioned, or a mitochondrial transit petide in case in Table I, application no. 2, in column 6 in the same line as SEQ ID NO. 61532 is depicted, mitochondrial is mentioned; which is operable linke, too.

In another embodiment thereof the expression cassette does not comprise an additional transit peptide in case in Table I, application no. 2, in column 6 in the same line as SEQ ID NO. 61532 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 61553, or a homolog or fragment thereof, or a homolog thereof being depicted in Table I, application no. 2, column 8, in the same line as SEQ ID NO. 61553, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 61553 is depicted, plastidic is mentioned, or a mitochondrial transit petide in case in Table I, application no. 2, in column 6 in the same line as SEQ ID NO. 61553 is depicted, mitochondrial is mentioned; which is operable linke, too.

In another embodiment thereof the expression cassette does not comprise an additional transit peptide in case in Table I, application no. 2, in column 6 in the same line as SEQ ID NO. 61553 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 61723, or a homolog or fragment thereof, or a homolog thereof being depicted in Table I, application no. 2, column 8, in the same line as SEQ ID NO. 61723, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 61723 is depicted, plastidic is mentioned, or a mitochondrial transit petide in case in Table I, application no. 2, in column 6 in the same line as SEQ ID NO. 61723 is depicted, mitochondrial is mentioned; which is operable linke, too.

In another embodiment thereof the expression cassette does not comprise an additional transit peptide in case in Table I, application no. 2, in column 6 in the same line as SEQ ID NO. 61723 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 62079, or a homolog or fragment thereof, or a homolog thereof being depicted in Table I, application no. 2, column 8, in the same line as SEQ ID NO. 62079, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 62079 is depicted, plastidic is mentioned, or a mitochondrial transit petide in case in Table I, application no. 2, in column 6 in the same line as SEQ ID NO. 62079 is depicted, mitochondrial is mentioned; which is operable linke, too.

In another embodiment thereof the expression cassette does not comprise an additional transit peptide in case in Table I, application no. 2, in column 6 in the same line as SEQ ID NO. 62079 is depicted, non-targeted is mentioned.

In an embodiment the present invention relates to an expresssion cassette comprising a) a promoter, prefereably a promoter selected from the group consisting of Big35S, PCUbi, Super and USP, in particular USP; and b) the nucleic acid SEQ ID NO. 41073, or a homolog or fragment thereof, or a homolog thereof being depicted in Table I, application no. 2, column 8, in the same line as SEQ ID NO. 41073, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 41073 is depicted, plastidic is mentioned, or a mitochondrial transit petide in case in Table I, application no. 2, in column 6 in the same line as SEQ ID NO. 41073 is depicted, mitochondrial is mentioned; which is operable linke, too.

In another embodiment thereof the expression cassette does not comprise an additional transit peptide in case in Table I, application no. 2, in column 6 in the same line as SEQ ID NO. 41073 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 62160, or a homolog or fragment thereof, or a homolog thereof being depicted in Table I, application no. 2, column 8, in the same line as SEQ ID NO. 62160, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 62160 is depicted, plastidic is mentioned, or a mitochondrial transit petide in case in Table I, application no. 2, in column 6 in the same line as SEQ ID NO. 62160 is depicted, mitochondrial is mentioned; which is operable linke, too.

In another embodiment thereof the expression cassette does not comprise an additional transit peptide in case in Table I, application no. 2, in column 6 in the same line as SEQ ID NO. 62160 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 59370, or a homolog or fragment thereof, or a homolog thereof being depicted in Table I, application no. 2, column 8, in the same line as SEQ ID NO. 59370, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 59370 is depicted, plastidic is mentioned, or a mitochondrial transit petide in case in Table I, application no. 2, in column 6 in the same line as SEQ ID NO. 59370 is depicted, mitochondrial is mentioned; which is operable linke, too.

In another embodiment thereof the expression cassette does not comprise an additional transit peptide in case in Table I, application no. 2, in column 6 in the same line as SEQ ID NO. 59370 is depicted, non-targeted is mentioned.

In an embodiment the present invention relates to an expresssion cassette comprising a) a promoter, prefereably a promoter selected from the group consisting of Big35S, PCUbi, Super and USP, in particular Big35S; and b) the nucleic acid SEQ ID NO. 62524, or a homolog or fragment thereof, or a homolog thereof being depicted in Table I, application no. 2, column 8, in the same line as SEQ ID NO. 62524, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 62524 is depicted, plastidic is mentioned, or a mitochondrial transit petide in case in Table I, application no. 2, in column 6 in the same line as SEQ ID NO. 62524 is depicted, mitochondrial is mentioned; which is operable linke, too.

In another embodiment thereof the expression cassette does not comprise an additional transit peptide in case in Table I, application no. 2, in column 6 in the same line as SEQ ID NO. 62524 is depicted, non-targeted is mentioned.

In an embodiment the present invention relates to an expresssion cassette comprising a) a promoter, prefereably a promoter selected from the group consisting of Big35S, PCUbi, Super and USP, in particular Big35S; and b) the nucleic acid SEQ ID NO. 62717, or a homolog or fragment thereof, or a homolog thereof being depicted in Table I, application no. 2, column 8, in the same line as SEQ ID NO. 62717, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 62717 is depicted, plastidic is mentioned, or a mitochondrial transit petide in case in Table I, application no. 2, in column 6 in the same line as SEQ ID NO. 62717 is depicted, mitochondrial is mentioned; which is operable linke, too.

In another embodiment thereof the expression cassette does not comprise an additional transit peptide in case in Table I, application no. 2, in column 6 in the same line as SEQ ID NO. 62717 is depicted, non-targeted is mentioned.

In an embodiment the present invention relates to an expresssion cassette comprising a) a promoter, prefereably a promoter selected from the group consisting of Big35S, PCUbi, Super and USP, in particular Big35S; and b) the nucleic acid SEQ ID NO. 63167, or a homolog or fragment thereof, or a homolog thereof being depicted in Table I, application no. 2, column 8, in the same line as SEQ ID NO. 63167, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 63167 is depicted, plastidic is mentioned, or a mitochondrial transit petide in case in Table I, application no. 2, in column 6 in the same line as SEQ ID NO. 63167 is depicted, mitochondrial is mentioned; which is operable linke, too.

In another embodiment thereof the expression cassette does not comprise an additional transit peptide in case in Table I, application no. 2, in column 6 in the same line as SEQ ID NO. 63167 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 63264, or a homolog or fragment thereof, or a homolog thereof being depicted in Table I, application no. 2, column 8, in the same line as SEQ ID NO. 63264, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 63264 is depicted, plastidic is mentioned, or a mitochondrial transit petide in case in Table I, application no. 2, in column 6 in the same line as SEQ ID NO. 63264 is depicted, mitochondrial is mentioned; which is operable linke, too.

In another embodiment thereof the expression cassette does not comprise an additional transit peptide in case in Table I, application no. 2, in column 6 in the same line as SEQ ID NO. 63264 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 14302, or a homolog or fragment thereof, or a homolog thereof being depicted in Table I, application no. 2, column 8, in the same line as SEQ ID NO. 14302, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 14302 is depicted, plastidic is mentioned, or a mitochondrial transit petide in case in Table I, application no. 2, in column 6 in the same line as SEQ ID NO. 14302 is depicted, mitochondrial is mentioned; which is operable linke, too.

In another embodiment thereof the expression cassette does not comprise an additional transit peptide in case in Table I, application no. 2, in column 6 in the same line as SEQ ID NO. 14302 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 63334, or a homolog or fragment thereof, or a homolog thereof being depicted in Table I, application no. 2, column 8, in the same line as SEQ ID NO. 63334, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 63334 is depicted, plastidic is mentioned, or a mitochondrial transit petide in case in Table I, application no. 2, in column 6 in the same line as SEQ ID NO. 63334 is depicted, mitochondrial is mentioned; which is operable linke, too.

In another embodiment thereof the expression cassette does not comprise an additional transit peptide in case in Table I, application no. 2, in column 6 in the same line as SEQ ID NO. 63334 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 63544, or a homolog or fragment thereof, or a homolog thereof being depicted in Table I, application no. 2, column 8, in the same line as SEQ ID NO. 63544, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 63544 is depicted, plastidic is mentioned, or a mitochondrial transit petide in case in Table I, application no. 2, in column 6 in the same line as SEQ ID NO. 63544 is depicted, mitochondrial is mentioned; which is operable linke, too.

In another embodiment thereof the expression cassette does not comprise an additional transit peptide in case in Table I, application no. 2, in column 6 in the same line as SEQ ID NO. 63544 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 12974, or a homolog or fragment thereof, or a homolog thereof being depicted in Table I, application no. 2, column 8, in the same line as SEQ ID NO. 12974, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 12974 is depicted, plastidic is mentioned, or a mitochondrial transit petide in case in Table I, application no. 2, in column 6 in the same line as SEQ ID NO. 12974 is depicted, mitochondrial is mentioned; which is operable linke, too.

In another embodiment thereof the expression cassette does not comprise an additional transit peptide in case in Table I, application no. 2, in column 6 in the same line as SEQ ID NO. 12974 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 58668, or a homolog or fragment thereof, or a homolog thereof being depicted in Table I, application no. 2, column 8, in the same line as SEQ ID NO. 58668, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 58668 is depicted, plastidic is mentioned, or a mitochondrial transit petide in case in Table I, application no. 2, in column 6 in the same line as SEQ ID NO. 58668 is depicted, mitochondrial is mentioned; which is operable linke, too.

In another embodiment thereof the expression cassette does not comprise an additional transit peptide in case in Table I, application no. 2, in column 6 in the same line as SEQ ID NO. 58668 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 56894, or a homolog or fragment thereof, or a homolog thereof being depicted in Table I, application no. 2, column 8, in the same line as SEQ ID NO. 56894, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 56894 is depicted, plastidic is mentioned, or a mitochondrial transit petide in case in Table I, application no. 2, in column 6 in the same line as SEQ ID NO. 56894 is depicted, mitochondrial is mentioned; which is operable linke, too.

In another embodiment thereof the expression cassette does not comprise an additional transit peptide in case in Table I, application no. 2, in column 6 in the same line as SEQ ID NO. 56894 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 57235, or a homolog or fragment thereof, or a homolog thereof being depicted in Table I, application no. 2, column 8, in the same line as SEQ ID NO. 57235, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 57235 is depicted, plastidic is mentioned, or a mitochondrial transit petide in case in Table I, application no. 2, in column 6 in the same line as SEQ ID NO. 57235 is depicted, mitochondrial is mentioned; which is operable linke, too.

In another embodiment thereof the expression cassette does not comprise an additional transit peptide in case in Table I, application no. 2, in column 6 in the same line as SEQ ID NO. 57235 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 57663, or a homolog or fragment thereof, or a homolog thereof being depicted in Table I, application no. 2, column 8, in the same line as SEQ ID NO. 57663, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 57663 is depicted, plastidic is mentioned, or a mitochondrial transit petide in case in Table I, application no. 2, in column 6 in the same line as SEQ ID NO. 57663 is depicted, mitochondrial is mentioned; which is operable linke, too.

In another embodiment thereof the expression cassette does not comprise an additional transit peptide in case in Table I, application no. 2, in column 6 in the same line as SEQ ID NO. 57663 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 57679, or a homolog or fragment thereof, or a homolog thereof being depicted in Table I, application no. 2, column 8, in the same line as SEQ ID NO. 57679, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 57679 is depicted, plastidic is mentioned, or a mitochondrial transit petide in case in Table I, application no. 2, in column 6 in the same line as SEQ ID NO. 57679 is depicted, mitochondrial is mentioned; which is operable linke, too.

In another embodiment thereof the expression cassette does not comprise an additional transit peptide in case in Table I, application no. 2, in column 6 in the same line as SEQ ID NO. 57679 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 57734, or a homolog or fragment thereof, or a homolog thereof being depicted in Table I, application no. 2, column 8, in the same line as SEQ ID NO. 57734, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 57734 is depicted, plastidic is mentioned, or a mitochondrial transit petide in case in Table I, application no. 2, in column 6 in the same line as SEQ ID NO. 57734 is depicted, mitochondrial is mentioned; which is operable linke, too.

In another embodiment thereof the expression cassette does not comprise an additional transit peptide in case in Table I, application no. 2, in column 6 in the same line as SEQ ID NO. 57734 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 58058, or a homolog or fragment thereof, or a homolog thereof being depicted in Table I, application no. 2, column 8, in the same line as SEQ ID NO. 58058, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 58058 is depicted, plastidic is mentioned, or a mitochondrial transit petide in case in Table I, application no. 2, in column 6 in the same line as SEQ ID NO. 58058 is depicted, mitochondrial is mentioned; which is operable linke, too.

In another embodiment thereof the expression cassette does not comprise an additional transit peptide in case in Table I, application no. 2, in column 6 in the same line as SEQ ID NO. 58058 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 58324, or a homolog or fragment thereof, or a homolog thereof being depicted in Table I, application no. 2, column 8, in the same line as SEQ ID NO. 58324, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 58324 is depicted, plastidic is mentioned, or a mitochondrial transit petide in case in Table I, application no. 2, in column 6 in the same line as SEQ ID NO. 58324 is depicted, mitochondrial is mentioned; which is operable linke, too.

In another embodiment thereof the expression cassette does not comprise an additional transit peptide in case in Table I, application no. 2, in column 6 in the same line as SEQ ID NO. 58324 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 12698, or a homolog or fragment thereof, or a homolog thereof being depicted in Table I, application no. 2, column 8, in the same line as SEQ ID NO. 12698, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 12698 is depicted, plastidic is mentioned, or a mitochondrial transit petide in case in Table I, application no. 2, in column 6 in the same line as SEQ ID NO. 12698 is depicted, mitochondrial is mentioned; which is operable linke, too.

In another embodiment thereof the expression cassette does not comprise an additional transit peptide in case in Table I, application no. 2, in column 6 in the same line as SEQ ID NO. 12698 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 58590, or a homolog or fragment thereof, or a homolog thereof being depicted in Table I, application no. 2, column 8, in the same line as SEQ ID NO. 58590, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 58590 is depicted, plastidic is mentioned, or a mitochondrial transit petide in case in Table I, application no. 2, in column 6 in the same line as SEQ ID NO. 58590 is depicted, mitochondrial is mentioned; which is operable linke, too.

In another embodiment thereof the expression cassette does not comprise an additional transit peptide in case in Table I, application no. 2, in column 6 in the same line as SEQ ID NO. 58590 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 59851, or a homolog or fragment thereof, or a homolog thereof being depicted in Table I, application no. 2, column 8, in the same line as SEQ ID NO. 59851, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 59851 is depicted, plastidic is mentioned, or a mitochondrial transit petide in case in Table I, application no. 2, in column 6 in the same line as SEQ ID NO. 59851 is depicted, mitochondrial is mentioned; which is operable linke, too.

In another embodiment thereof the expression cassette does not comprise an additional transit peptide in case in Table I, application no. 2, in column 6 in the same line as SEQ ID NO. 59851 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 12070, or a homolog or fragment thereof, or a homolog thereof being depicted in Table I, application no. 2, column 8, in the same line as SEQ ID NO. 12070, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 12070 is depicted, plastidic is mentioned, or a mitochondrial transit petide in case in Table I, application no. 2, in column 6 in the same line as SEQ ID NO. 12070 is depicted, mitochondrial is mentioned; which is operable linke, too.

In another embodiment thereof the expression cassette does not comprise an additional transit peptide in case in Table I, application no. 2, in column 6 in the same line as SEQ ID NO. 12070 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 58731, or a homolog or fragment thereof, or a homolog thereof being depicted in Table I, application no. 2, column 8, in the same line as SEQ ID NO. 58731, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 58731 is depicted, plastidic is mentioned, or a mitochondrial transit petide in case in Table I, application no. 2, in column 6 in the same line as SEQ ID NO. 58731 is depicted, mitochondrial is mentioned; which is operable linke, too.

In another embodiment thereof the expression cassette does not comprise an additional transit peptide in case in Table I, application no. 2, in column 6 in the same line as SEQ ID NO. 58731 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 58751, or a homolog or fragment thereof, or a homolog thereof being depicted in Table I, application no. 2, column 8, in the same line as SEQ ID NO. 58751, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 58751 is depicted, plastidic is mentioned, or a mitochondrial transit petide in case in Table I, application no. 2, in column 6 in the same line as SEQ ID NO. 58751 is depicted, mitochondrial is mentioned; which is operable linke, too.

In another embodiment thereof the expression cassette does not comprise an additional transit peptide in case in Table I, application no. 2, in column 6 in the same line as SEQ ID NO. 58751 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 58823, or a homolog or fragment thereof, or a homolog thereof being depicted in Table I, application no. 2, column 8, in the same line as SEQ ID NO. 58823, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 58823 is depicted, plastidic is mentioned, or a mitochondrial transit petide in case in Table I, application no. 2, in column 6 in the same line as SEQ ID NO. 58823 is depicted, mitochondrial is mentioned; which is operable linke, too.

In another embodiment thereof the expression cassette does not comprise an additional transit peptide in case in Table I, application no. 2, in column 6 in the same line as SEQ ID NO. 58823 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 12140, or a homolog or fragment thereof, or a homolog thereof being depicted in Table I, application no. 2, column 8, in the same line as SEQ ID NO. 12140, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 12140 is depicted, plastidic is mentioned, or a mitochondrial transit petide in case in Table I, application no. 2, in column 6 in the same line as SEQ ID NO. 12140 is depicted, mitochondrial is mentioned; which is operable linke, too.

In another embodiment thereof the expression cassette does not comprise an additional transit peptide in case in Table I, application no. 2, in column 6 in the same line as SEQ ID NO. 12140 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 59041, or a homolog or fragment thereof, or a homolog thereof being depicted in Table I, application no. 2, column 8, in the same line as SEQ ID NO. 59041, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 59041 is depicted, plastidic is mentioned, or a mitochondrial transit petide in case in Table I, application no. 2, in column 6 in the same line as SEQ ID NO. 59041 is depicted, mitochondrial is mentioned; which is operable linke, too.

In another embodiment thereof the expression cassette does not comprise an additional transit peptide in case in Table I, application no. 2, in column 6 in the same line as SEQ ID NO. 59041 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 59165, or a homolog or fragment thereof, or a homolog thereof being depicted in Table I, application no. 2, column 8, in the same line as SEQ ID NO. 59165, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 59165 is depicted, plastidic is mentioned, or a mitochondrial transit petide in case in Table I, application no. 2, in column 6 in the same line as SEQ ID NO. 59165 is depicted, mitochondrial is mentioned; which is operable linke, too.

In another embodiment thereof the expression cassette does not comprise an additional transit peptide in case in Table I, application no. 2, in column 6 in the same line as SEQ ID NO. 59165 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 63745, or a homolog or fragment thereof, or a homolog thereof being depicted in Table I, application no. 2, column 8, in the same line as SEQ ID NO. 63745, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 63745 is depicted, plastidic is mentioned, or a mitochondrial transit petide in case in Table I, application no. 2, in column 6 in the same line as SEQ ID NO. 63745 is depicted, mitochondrial is mentioned; which is operable linke, too.

In another embodiment thereof the expression cassette does not comprise an additional transit peptide in case in Table I, application no. 2, in column 6 in the same line as SEQ ID NO. 63745 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 58472, or a homolog or fragment thereof, or a homolog thereof being depicted in Table I, application no. 2, column 8, in the same line as SEQ ID NO. 58472, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 58472 is depicted, plastidic is mentioned, or a mitochondrial transit petide in case in Table I, application no. 2, in column 6 in the same line as SEQ ID NO. 58472 is depicted, mitochondrial is mentioned; which is operable linke, too.

In another embodiment thereof the expression cassette does not comprise an additional transit peptide in case in Table I, application no. 2, in column 6 in the same line as SEQ ID NO. 58472 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 67299, or a homolog or fragment thereof, or a homolog thereof being depicted in Table I, application no. 2, column 8, in the same line as SEQ ID NO. 67299, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 67299 is depicted, plastidic is mentioned, or a mitochondrial transit petide in case in Table I, application no. 2, in column 6 in the same line as SEQ ID NO. 67299 is depicted, mitochondrial is mentioned; which is operable linke, too.

In another embodiment thereof the expression cassette does not comprise an additional transit peptide in case in Table I, application no. 2, in column 6 in the same line as SEQ ID NO. 67299 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 63665, or a homolog or fragment thereof, or a homolog thereof being depicted in Table I, application no. 2, column 8, in the same line as SEQ ID NO. 63665, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 63665 is depicted, plastidic is mentioned, or a mitochondrial transit petide in case in Table I, application no. 2, in column 6 in the same line as SEQ ID NO. 63665 is depicted, mitochondrial is mentioned; which is operable linke, too.

In another embodiment thereof the expression cassette does not comprise an additional transit peptide in case in Table I, application no. 2, in column 6 in the same line as SEQ ID NO. 63665 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 65224, or a homolog or fragment thereof, or a homolog thereof being depicted in Table I, application no. 2, column 8, in the same line as SEQ ID NO. 65224, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 65224 is depicted, plastidic is mentioned, or a mitochondrial transit petide in case in Table I, application no. 2, in column 6 in the same line as SEQ ID NO. 65224 is depicted, mitochondrial is mentioned; which is operable linke, too.

In another embodiment thereof the expression cassette does not comprise an additional transit peptide in case in Table I, application no. 2, in column 6 in the same line as SEQ ID NO. 65224 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 66225, or a homolog or fragment thereof, or a homolog thereof being depicted in Table I, application no. 2, column 8, in the same line as SEQ ID NO. 66225, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 66225 is depicted, plastidic is mentioned, or a mitochondrial transit petide in case in Table I, application no. 2, in column 6 in the same line as SEQ ID NO. 66225 is depicted, mitochondrial is mentioned; which is operable linke, too.

In another embodiment thereof the expression cassette does not comprise an additional transit peptide in case in Table I, application no. 2, in column 6 in the same line as SEQ ID NO. 66225 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 66274, or a homolog or fragment thereof, or a homolog thereof being depicted in Table I, application no. 2, column 8, in the same line as SEQ ID NO. 66274, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 66274 is depicted, plastidic is mentioned, or a mitochondrial transit petide in case in Table I, application no. 2, in column 6 in the same line as SEQ ID NO. 66274 is depicted, mitochondrial is mentioned; which is operable linke, too.

In another embodiment thereof the expression cassette does not comprise an additional transit peptide in case in Table I, application no. 2, in column 6 in the same line as SEQ ID NO. 66274 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 66419, or a homolog or fragment thereof, or a homolog thereof being depicted in Table I, application no. 2, column 8, in the same line as SEQ ID NO. 66419, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 66419 is depicted, plastidic is mentioned, or a mitochondrial transit petide in case in Table I, application no. 2, in column 6 in the same line as SEQ ID NO. 66419 is depicted, mitochondrial is mentioned; which is operable linke, too.

In another embodiment thereof the expression cassette does not comprise an additional transit peptide in case in Table I, application no. 2, in column 6 in the same line as SEQ ID NO. 66419 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 66695, or a homolog or fragment thereof, or a homolog thereof being depicted in Table I, application no. 2, column 8, in the same line as SEQ ID NO. 66695, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 66695 is depicted, plastidic is mentioned, or a mitochondrial transit petide in case in Table I, application no. 2, in column 6 in the same line as SEQ ID NO. 66695 is depicted, mitochondrial is mentioned; which is operable linke, too.

In another embodiment thereof the expression cassette does not comprise an additional transit peptide in case in Table I, application no. 2, in column 6 in the same line as SEQ ID NO. 66695 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 66715, or a homolog or fragment thereof, or a homolog thereof being depicted in Table I, application no. 2, column 8, in the same line as SEQ ID NO. 66715, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 66715 is depicted, plastidic is mentioned, or a mitochondrial transit petide in case in Table I, application no. 2, in column 6 in the same line as SEQ ID NO. 66715 is depicted, mitochondrial is mentioned; which is operable linke, too.

In another embodiment thereof the expression cassette does not comprise an additional transit peptide in case in Table I, application no. 2, in column 6 in the same line as SEQ ID NO. 66715 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 65181, or a homolog or fragment thereof, or a homolog thereof being depicted in Table I, application no. 2, column 8, in the same line as SEQ ID NO. 65181, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 65181 is depicted, plastidic is mentioned, or a mitochondrial transit petide in case in Table I, application no. 2, in column 6 in the same line as SEQ ID NO. 65181 is depicted, mitochondrial is mentioned; which is operable linke, too.

In another embodiment thereof the expression cassette does not comprise an additional transit peptide in case in Table I, application no. 2, in column 6 in the same line as SEQ ID NO. 65181 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 67190, or a homolog or fragment thereof, or a homolog thereof being depicted in Table I, application no. 2, column 8, in the same line as SEQ ID NO. 67190, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 67190 is depicted, plastidic is mentioned, or a mitochondrial transit petide in case in Table I, application no. 2, in column 6 in the same line as SEQ ID NO. 67190 is depicted, mitochondrial is mentioned; which is operable linke, too.

In another embodiment thereof the expression cassette does not comprise an additional transit peptide in case in Table I, application no. 2, in column 6 in the same line as SEQ ID NO. 67190 is depicted, non-targeted is mentioned.

In an embodiment the present invention relates to an expresssion cassette comprising a) a promoter, prefereably a promoter selected from the group consisting of Big35S, PCUbi, Super and USP, in particular Big35S; and b) the nucleic acid SEQ ID NO. 64975, or a homolog or fragment thereof, or a homolog thereof being depicted in Table I, application no. 2, column 8, in the same line as SEQ ID NO. 64975, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 64975 is depicted, plastidic is mentioned, or a mitochondrial transit petide in case in Table I, application no. 2, in column 6 in the same line as SEQ ID NO. 64975 is depicted, mitochondrial is mentioned; which is operable linke, too.

In another embodiment thereof the expression cassette does not comprise an additional transit peptide in case in Table I, application no. 2, in column 6 in the same line as SEQ ID NO. 64975 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 67646, or a homolog or fragment thereof, or a homolog thereof being depicted in Table I, application no. 2, column 8, in the same line as SEQ ID NO. 67646, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 67646 is depicted, plastidic is mentioned, or a mitochondrial transit petide in case in Table I, application no. 2, in column 6 in the same line as SEQ ID NO. 67646 is depicted, mitochondrial is mentioned; which is operable linke, too.

In another embodiment thereof the expression cassette does not comprise an additional transit peptide in case in Table I, application no. 2, in column 6 in the same line as SEQ ID NO. 67646 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 67684, or a homolog or fragment thereof, or a homolog thereof being depicted in Table I, application no. 2, column 8, in the same line as SEQ ID NO. 67684, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 67684 is depicted, plastidic is mentioned, or a mitochondrial transit petide in case in Table I, application no. 2, in column 6 in the same line as SEQ ID NO. 67684 is depicted, mitochondrial is mentioned; which is operable linke, too.

In another embodiment thereof the expression cassette does not comprise an additional transit peptide in case in Table I, application no. 2, in column 6 in the same line as SEQ ID NO. 67684 is depicted, non-targeted is mentioned.

In an embodiment the present invention relates to an expresssion cassette comprising a) a promoter, prefereably a promoter selected from the group consisting of Big35S, PCUbi, Super and USP, in particular Big35S; and b) the nucleic acid SEQ ID NO. 67710, or a homolog or fragment thereof, or a homolog thereof being depicted in Table I, application no. 2, column 8, in the same line as SEQ ID NO. 67710, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 67710 is depicted, plastidic is mentioned, or a mitochondrial transit petide in case in Table I, application no. 2, in column 6 in the same line as SEQ ID NO. 67710 is depicted, mitochondrial is mentioned; which is operable linke, too.

In another embodiment thereof the expression cassette does not comprise an additional transit peptide in case in Table I, application no. 2, in column 6 in the same line as SEQ ID NO. 67710 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 67951, or a homolog or fragment thereof, or a homolog thereof being depicted in Table I, application no. 2, column 8, in the same line as SEQ ID NO. 67951, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 67951 is depicted, plastidic is mentioned, or a mitochondrial transit petide in case in Table I, application no. 2, in column 6 in the same line as SEQ ID NO. 67951 is depicted, mitochondrial is mentioned; which is operable linke, too.

In another embodiment thereof the expression cassette does not comprise an additional transit peptide in case in Table I, application no. 2, in column 6 in the same line as SEQ ID NO. 67951 is depicted, non-targeted is mentioned.

In an embodiment the present invention relates to an expresssion cassette comprising a) a promoter, prefereably a promoter selected from the group consisting of Big35S, PCUbi, Super and USP, in particular Big35S; and b) the nucleic acid SEQ ID NO. 67968, or a homolog or fragment thereof, or a homolog thereof being depicted in Table I, application no. 2, column 8, in the same line as SEQ ID NO. 67968, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 67968 is depicted, plastidic is mentioned, or a mitochondrial transit petide in case in Table I, application no. 2, in column 6 in the same line as SEQ ID NO. 67968 is depicted, mitochondrial is mentioned; which is operable linke, too.

In another embodiment thereof the expression cassette does not comprise an additional transit peptide in case in Table I, application no. 2, in column 6 in the same line as SEQ ID NO. 67968 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 67998, or a homolog or fragment thereof, or a homolog thereof being depicted in Table I, application no. 2, column 8, in the same line as SEQ ID NO. 67998, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 67998 is depicted, plastidic is mentioned, or a mitochondrial transit petide in case in Table I, application no. 2, in column 6 in the same line as SEQ ID NO. 67998 is depicted, mitochondrial is mentioned; which is operable linke, too.

In another embodiment thereof the expression cassette does not comprise an additional transit peptide in case in Table I, application no. 2, in column 6 in the same line as SEQ ID NO. 67998 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 68132, or a homolog or fragment thereof, or a homolog thereof being depicted in Table I, application no. 2, column 8, in the same line as SEQ ID NO. 68132, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 68132 is depicted, plastidic is mentioned, or a mitochondrial transit petide in case in Table I, application no. 2, in column 6 in the same line as SEQ ID NO. 68132 is depicted, mitochondrial is mentioned; which is operable linke, too.

In another embodiment thereof the expression cassette does not comprise an additional transit peptide in case in Table I, application no. 2, in column 6 in the same line as SEQ ID NO. 68132 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 68363, or a homolog or fragment thereof, or a homolog thereof being depicted in Table I, application no. 2, column 8, in the same line as SEQ ID NO. 68363, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 68363 is depicted, plastidic is mentioned, or a mitochondrial transit petide in case in Table I, application no. 2, in column 6 in the same line as SEQ ID NO. 68363 is depicted, mitochondrial is mentioned; which is operable linke, too.

In another embodiment thereof the expression cassette does not comprise an additional transit peptide in case in Table I, application no. 2, in column 6 in the same line as SEQ ID NO. 68363 is depicted, non-targeted is mentioned.

In an embodiment the present invention relates to an expresssion cassette comprising a) a promoter, prefereably a promoter selected from the group consisting of Big35S, PCUbi, Super and USP, in particular Big35S; and b) the nucleic acid SEQ ID NO. 66772, or a homolog or fragment thereof, or a homolog thereof being depicted in Table I, application no. 2, column 8, in the same line as SEQ ID NO. 66772, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 66772 is depicted, plastidic is mentioned, or a mitochondrial transit petide in case in Table I, application no. 2, in column 6 in the same line as SEQ ID NO. 66772 is depicted, mitochondrial is mentioned; which is operable linke, too.

In another embodiment thereof the expression cassette does not comprise an additional transit peptide in case in Table I, application no. 2, in column 6 in the same line as SEQ ID NO. 66772 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 64315, or a homolog or fragment thereof, or a homolog thereof being depicted in Table I, application no. 2, column 8, in the same line as SEQ ID NO. 64315, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 64315 is depicted, plastidic is mentioned, or a mitochondrial transit petide in case in Table I, application no. 2, in column 6 in the same line as SEQ ID NO. 64315 is depicted, mitochondrial is mentioned; which is operable linke, too.

In another embodiment thereof the expression cassette does not comprise an additional transit peptide in case in Table I, application no. 2, in column 6 in the same line as SEQ ID NO. 64315 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 56153, or a homolog or fragment thereof, or a homolog thereof being depicted in Table I, application no. 2, column 8, in the same line as SEQ ID NO. 56153, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 56153 is depicted, plastidic is mentioned, or a mitochondrial transit petide in case in Table I, application no. 2, in column 6 in the same line as SEQ ID NO. 56153 is depicted, mitochondrial is mentioned; which is operable linke, too.

In another embodiment thereof the expression cassette does not comprise an additional transit peptide in case in Table I, application no. 2, in column 6 in the same line as SEQ ID NO. 56153 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 63803, or a homolog or fragment thereof, or a homolog thereof being depicted in Table I, application no. 2, column 8, in the same line as SEQ ID NO. 63803, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 63803 is depicted, plastidic is mentioned, or a mitochondrial transit petide in case in Table I, application no. 2, in column 6 in the same line as SEQ ID NO. 63803 is depicted, mitochondrial is mentioned; which is operable linke, too.

In another embodiment thereof the expression cassette does not comprise an additional transit peptide in case in Table I, application no. 2, in column 6 in the same line as SEQ ID NO. 63803 is depicted, non-targeted is mentioned.

In an embodiment the present invention relates to an expresssion cassette comprising a) a promoter, prefereably a promoter selected from the group consisting of Big35S, PCUbi, Super and USP, in particular Big35S; and b) the nucleic acid SEQ ID NO. 63807, or a homolog or fragment thereof, or a homolog thereof being depicted in Table I, application no. 2, column 8, in the same line as SEQ ID NO. 63807, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 63807 is depicted, plastidic is mentioned, or a mitochondrial transit petide in case in Table I, application no. 2, in column 6 in the same line as SEQ ID NO. 63807 is depicted, mitochondrial is mentioned; which is operable linke, too.

In another embodiment thereof the expression cassette does not comprise an additional transit peptide in case in Table I, application no. 2, in column 6 in the same line as SEQ ID NO. 63807 is depicted, non-targeted is mentioned.

In an embodiment the present invention relates to an expresssion cassette comprising a) a promoter, prefereably a promoter selected from the group consisting of Big35S, PCUbi, Super and USP, in particular Big35S; and b) the nucleic acid SEQ ID NO. 64144, or a homolog or fragment thereof, or a homolog thereof being depicted in Table I, application no. 2, column 8, in the same line as SEQ ID NO. 64144, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 64144 is depicted, plastidic is mentioned, or a mitochondrial transit petide in case in Table I, application no. 2, in column 6 in the same line as SEQ ID NO. 64144 is depicted, mitochondrial is mentioned; which is operable linke, too.

In another embodiment thereof the expression cassette does not comprise an additional transit peptide in case in Table I, application no. 2, in column 6 in the same line as SEQ ID NO. 64144 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 64148, or a homolog or fragment thereof, or a homolog thereof being depicted in Table I, application no. 2, column 8, in the same line as SEQ ID NO. 64148, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 64148 is depicted, plastidic is mentioned, or a mitochondrial transit petide in case in Table I, application no. 2, in column 6 in the same line as SEQ ID NO. 64148 is depicted, mitochondrial is mentioned; which is operable linke, too.

In another embodiment thereof the expression cassette does not comprise an additional transit peptide in case in Table I, application no. 2, in column 6 in the same line as SEQ ID NO. 64148 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 64157, or a homolog or fragment thereof, or a homolog thereof being depicted in Table I, application no. 2, column 8, in the same line as SEQ ID NO. 64157, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 64157 is depicted, plastidic is mentioned, or a mitochondrial transit petide in case in Table I, application no. 2, in column 6 in the same line as SEQ ID NO. 64157 is depicted, mitochondrial is mentioned; which is operable linke, too.

In another embodiment thereof the expression cassette does not comprise an additional transit peptide in case in Table I, application no. 2, in column 6 in the same line as SEQ ID NO. 64157 is depicted, non-targeted is mentioned.

In an embodiment the present invention relates to an expresssion cassette comprising a) a promoter, prefereably a promoter selected from the group consisting of Big35S, PCUbi, Super and USP, in particular Big35S; and b) the nucleic acid SEQ ID NO. 64177, or a homolog or fragment thereof, or a homolog thereof being depicted in Table I, application no. 2, column 8, in the same line as SEQ ID NO. 64177, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 64177 is depicted, plastidic is mentioned, or a mitochondrial transit petide in case in Table I, application no. 2, in column 6 in the same line as SEQ ID NO. 64177 is depicted, mitochondrial is mentioned; which is operable linke, too.

In another embodiment thereof the expression cassette does not comprise an additional transit peptide in case in Table I, application no. 2, in column 6 in the same line as SEQ ID NO. 64177 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 14821, or a homolog or fragment thereof, or a homolog thereof being depicted in Table I, application no. 2, column 8, in the same line as SEQ ID NO. 14821, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 14821 is depicted, plastidic is mentioned, or a mitochondrial transit petide in case in Table I, application no. 2, in column 6 in the same line as SEQ ID NO. 14821 is depicted, mitochondrial is mentioned; which is operable linke, too.

In another embodiment thereof the expression cassette does not comprise an additional transit peptide in case in Table I, application no. 2, in column 6 in the same line as SEQ ID NO. 14821 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 64218, or a homolog or fragment thereof, or a homolog thereof being depicted in Table I, application no. 2, column 8, in the same line as SEQ ID NO. 64218, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 64218 is depicted, plastidic is mentioned, or a mitochondrial transit petide in case in Table I, application no. 2, in column 6 in the same line as SEQ ID NO. 64218 is depicted, mitochondrial is mentioned; which is operable linke, too.

In another embodiment thereof the expression cassette does not comprise an additional transit peptide in case in Table I, application no. 2, in column 6 in the same line as SEQ ID NO. 64218 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 63713, or a homolog or fragment thereof, or a homolog thereof being depicted in Table I, application no. 2, column 8, in the same line as SEQ ID NO. 63713, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 63713 is depicted, plastidic is mentioned, or a mitochondrial transit petide in case in Table I, application no. 2, in column 6 in the same line as SEQ ID NO. 63713 is depicted, mitochondrial is mentioned; which is operable linke, too.

In another embodiment thereof the expression cassette does not comprise an additional transit peptide in case in Table I, application no. 2, in column 6 in the same line as SEQ ID NO. 63713 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 14715, or a homolog or fragment thereof, or a homolog thereof being depicted in Table I, application no. 2, column 8, in the same line as SEQ ID NO. 14715, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 14715 is depicted, plastidic is mentioned, or a mitochondrial transit petide in case in Table I, application no. 2, in column 6 in the same line as SEQ ID NO. 14715 is depicted, mitochondrial is mentioned; which is operable linke, too.

In another embodiment thereof the expression cassette does not comprise an additional transit peptide in case in Table I, application no. 2, in column 6 in the same line as SEQ ID NO. 14715 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 64336, or a homolog or fragment thereof, or a homolog thereof being depicted in Table I, application no. 2, column 8, in the same line as SEQ ID NO. 64336, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 64336 is depicted, plastidic is mentioned, or a mitochondrial transit petide in case in Table I, application no. 2, in column 6 in the same line as SEQ ID NO. 64336 is depicted, mitochondrial is mentioned; which is operable linke, too.

In another embodiment thereof the expression cassette does not comprise an additional transit peptide in case in Table I, application no. 2, in column 6 in the same line as SEQ ID NO. 64336 is depicted, non-targeted is mentioned.

In an embodiment the present invention relates to an expresssion cassette comprising a) a promoter, prefereably a promoter selected from the group consisting of Big35S, PCUbi, Super and USP, in particular USP; and b) the nucleic acid SEQ ID NO. 64470, or a homolog or fragment thereof, or a homolog thereof being depicted in Table I, application no. 2, column 8, in the same line as SEQ ID NO. 64470, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 64470 is depicted, plastidic is mentioned, or a mitochondrial transit petide in case in Table I, application no. 2, in column 6 in the same line as SEQ ID NO. 64470 is depicted, mitochondrial is mentioned; which is operable linke, too.

In another embodiment thereof the expression cassette does not comprise an additional transit peptide in case in Table I, application no. 2, in column 6 in the same line as SEQ ID NO. 64470 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 64546, or a homolog or fragment thereof, or a homolog thereof being depicted in Table I, application no. 2, column 8, in the same line as SEQ ID NO. 64546, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 64546 is depicted, plastidic is mentioned, or a mitochondrial transit petide in case in Table I, application no. 2, in column 6 in the same line as SEQ ID NO. 64546 is depicted, mitochondrial is mentioned; which is operable linke, too.

In another embodiment thereof the expression cassette does not comprise an additional transit peptide in case in Table I, application no. 2, in column 6 in the same line as SEQ ID NO. 64546 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 64563, or a homolog or fragment thereof, or a homolog thereof being depicted in Table I, application no. 2, column 8, in the same line as SEQ ID NO. 64563, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 64563 is depicted, plastidic is mentioned, or a mitochondrial transit petide in case in Table I, application no. 2, in column 6 in the same line as SEQ ID NO. 64563 is depicted, mitochondrial is mentioned; which is operable linke, too.

In another embodiment thereof the expression cassette does not comprise an additional transit peptide in case in Table I, application no. 2, in column 6 in the same line as SEQ ID NO. 64563 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 64773, or a homolog or fragment thereof, or a homolog thereof being depicted in Table I, application no. 2, column 8, in the same line as SEQ ID NO. 64773, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 64773 is depicted, plastidic is mentioned, or a mitochondrial transit petide in case in Table I, application no. 2, in column 6 in the same line as SEQ ID NO. 64773 is depicted, mitochondrial is mentioned; which is operable linke, too.

In another embodiment thereof the expression cassette does not comprise an additional transit peptide in case in Table I, application no. 2, in column 6 in the same line as SEQ ID NO. 64773 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 64894, or a homolog or fragment thereof, or a homolog thereof being depicted in Table I, application no. 2, column 8, in the same line as SEQ ID NO. 64894, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 64894 is depicted, plastidic is mentioned, or a mitochondrial transit petide in case in Table I, application no. 2, in column 6 in the same line as SEQ ID NO. 64894 is depicted, mitochondrial is mentioned; which is operable linke, too.

In another embodiment thereof the expression cassette does not comprise an additional transit peptide in case in Table I, application no. 2, in column 6 in the same line as SEQ ID NO. 64894 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 64964, or a homolog or fragment thereof, or a homolog thereof being depicted in Table I, application no. 2, column 8, in the same line as SEQ ID NO. 64964, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 64964 is depicted, plastidic is mentioned, or a mitochondrial transit petide in case in Table I, application no. 2, in column 6 in the same line as SEQ ID NO. 64964 is depicted, mitochondrial is mentioned; which is operable linke, too.

In another embodiment thereof the expression cassette does not comprise an additional transit peptide in case in Table I, application no. 2, in column 6 in the same line as SEQ ID NO. 64964 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 64198, or a homolog or fragment thereof, or a homolog thereof being depicted in Table I, application no. 2, column 8, in the same line as SEQ ID NO. 64198, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 64198 is depicted, plastidic is mentioned, or a mitochondrial transit petide in case in Table I, application no. 2, in column 6 in the same line as SEQ ID NO. 64198 is depicted, mitochondrial is mentioned; which is operable linke, too.

In another embodiment thereof the expression cassette does not comprise an additional transit peptide in case in Table I, application no. 2, in column 6 in the same line as SEQ ID NO. 64198 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 45394, or a homolog or fragment thereof, or a homolog thereof being depicted in Table I, application no. 2, column 8, in the same line as SEQ ID NO. 45394, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 45394 is depicted, plastidic is mentioned, or a mitochondrial transit petide in case in Table I, application no. 2, in column 6 in the same line as SEQ ID NO. 45394 is depicted, mitochondrial is mentioned; which is operable linke, too.

In another embodiment thereof the expression cassette does not comprise an additional transit peptide in case in Table I, application no. 2, in column 6 in the same line as SEQ ID NO. 45394 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 46850, or a homolog or fragment thereof, or a homolog thereof being depicted in Table I, application no. 2, column 8, in the same line as SEQ ID NO. 46850, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 46850 is depicted, plastidic is mentioned, or a mitochondrial transit petide in case in Table I, application no. 2, in column 6 in the same line as SEQ ID NO. 46850 is depicted, mitochondrial is mentioned; which is operable linke, too.

In another embodiment thereof the expression cassette does not comprise an additional transit peptide in case in Table I, application no. 2, in column 6 in the same line as SEQ ID NO. 46850 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 44372, or a homolog or fragment thereof, or a homolog thereof being depicted in Table I, application no. 2, column 8, in the same line as SEQ ID NO. 44372, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 44372 is depicted, plastidic is mentioned, or a mitochondrial transit petide in case in Table I, application no. 2, in column 6 in the same line as SEQ ID NO. 44372 is depicted, mitochondrial is mentioned; which is operable linke, too.

In another embodiment thereof the expression cassette does not comprise an additional transit peptide in case in Table I, application no. 2, in column 6 in the same line as SEQ ID NO. 44372 is depicted, non-targeted is mentioned.

In an embodiment the present invention relates to an expresssion cassette comprising a) a promoter, prefereably a promoter selected from the group consisting of Big35S, PCUbi, Super and USP, in particular USP; and b) the nucleic acid SEQ ID NO. 44378, or a homolog or fragment thereof, or a homolog thereof being depicted in Table I, application no. 2, column 8, in the same line as SEQ ID NO. 44378, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 44378 is depicted, plastidic is mentioned, or a mitochondrial transit petide in case in Table I, application no. 2, in column 6 in the same line as SEQ ID NO. 44378 is depicted, mitochondrial is mentioned; which is operable linke, too.

In another embodiment thereof the expression cassette does not comprise an additional transit peptide in case in Table I, application no. 2, in column 6 in the same line as SEQ ID NO. 44378 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 10740, or a homolog or fragment thereof, or a homolog thereof being depicted in Table I, application no. 2, column 8, in the same line as SEQ ID NO. 10740, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 10740 is depicted, plastidic is mentioned, or a mitochondrial transit petide in case in Table I, application no. 2, in column 6 in the same line as SEQ ID NO. 10740 is depicted, mitochondrial is mentioned; which is operable linke, too.

In another embodiment thereof the expression cassette does not comprise an additional transit peptide in case in Table I, application no. 2, in column 6 in the same line as SEQ ID NO. 10740 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 44466, or a homolog or fragment thereof, or a homolog thereof being depicted in Table I, application no. 2, column 8, in the same line as SEQ ID NO. 44466, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 44466 is depicted, plastidic is mentioned, or a mitochondrial transit petide in case in Table I, application no. 2, in column 6 in the same line as SEQ ID NO. 44466 is depicted, mitochondrial is mentioned; which is operable linke, too.

In another embodiment thereof the expression cassette does not comprise an additional transit peptide in case in Table I, application no. 2, in column 6 in the same line as SEQ ID NO. 44466 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 44609, or a homolog or fragment thereof, or a homolog thereof being depicted in Table I, application no. 2, column 8, in the same line as SEQ ID NO. 44609, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 44609 is depicted, plastidic is mentioned, or a mitochondrial transit petide in case in Table I, application no. 2, in column 6 in the same line as SEQ ID NO. 44609 is depicted, mitochondrial is mentioned; which is operable linke, too.

In another embodiment thereof the expression cassette does not comprise an additional transit peptide in case in Table I, application no. 2, in column 6 in the same line as SEQ ID NO. 44609 is depicted, non-targeted is mentioned.

In an embodiment the present invention relates to an expresssion cassette comprising a) a promoter, prefereably a promoter selected from the group consisting of Big35S, PCUbi, Super and USP, in particular USP; and b) the nucleic acid SEQ ID NO. 44662, or a homolog or fragment thereof, or a homolog thereof being depicted in Table I, application no. 2, column 8, in the same line as SEQ ID NO. 44662, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 44662 is depicted, plastidic is mentioned, or a mitochondrial transit petide in case in Table I, application no. 2, in column 6 in the same line as SEQ ID NO. 44662 is depicted, mitochondrial is mentioned; which is operable linke, too.

In another embodiment thereof the expression cassette does not comprise an additional transit peptide in case in Table I, application no. 2, in column 6 in the same line as SEQ ID NO. 44662 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 10708, or a homolog or fragment thereof, or a homolog thereof being depicted in Table I, application no. 2, column 8, in the same line as SEQ ID NO. 10708, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 10708 is depicted, plastidic is mentioned, or a mitochondrial transit petide in case in Table I, application no. 2, in column 6 in the same line as SEQ ID NO. 10708 is depicted, mitochondrial is mentioned; which is operable linke, too.

In another embodiment thereof the expression cassette does not comprise an additional transit peptide in case in Table I, application no. 2, in column 6 in the same line as SEQ ID NO. 10708 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 45321, or a homolog or fragment thereof, or a homolog thereof being depicted in Table I, application no. 2, column 8, in the same line as SEQ ID NO. 45321, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 45321 is depicted, plastidic is mentioned, or a mitochondrial transit petide in case in Table I, application no. 2, in column 6 in the same line as SEQ ID NO. 45321 is depicted, mitochondrial is mentioned; which is operable linke, too.

In another embodiment thereof the expression cassette does not comprise an additional transit peptide in case in Table I, application no. 2, in column 6 in the same line as SEQ ID NO. 45321 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 44196, or a homolog or fragment thereof, or a homolog thereof being depicted in Table I, application no. 2, column 8, in the same line as SEQ ID NO. 44196, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 44196 is depicted, plastidic is mentioned, or a mitochondrial transit petide in case in Table I, application no. 2, in column 6 in the same line as SEQ ID NO. 44196 is depicted, mitochondrial is mentioned; which is operable linke, too.

In another embodiment thereof the expression cassette does not comprise an additional transit peptide in case in Table I, application no. 2, in column 6 in the same line as SEQ ID NO. 44196 is depicted, non-targeted is mentioned.

In an embodiment the present invention relates to an expresssion cassette comprising a) a promoter, prefereably a promoter selected from the group consisting of Big35S, PCUbi, Super and USP, in particular USP; and b) the nucleic acid SEQ ID NO. 45556, or a homolog or fragment thereof, or a homolog thereof being depicted in Table I, application no. 2, column 8, in the same line as SEQ ID NO. 45556, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 45556 is depicted, plastidic is mentioned, or a mitochondrial transit petide in case in Table I, application no. 2, in column 6 in the same line as SEQ ID NO. 45556 is depicted, mitochondrial is mentioned; which is operable linke, too.

In another embodiment thereof the expression cassette does not comprise an additional transit peptide in case in Table I, application no. 2, in column 6 in the same line as SEQ ID NO. 45556 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 45757, or a homolog or fragment thereof, or a homolog thereof being depicted in Table I, application no. 2, column 8, in the same line as SEQ ID NO. 45757, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 45757 is depicted, plastidic is mentioned, or a mitochondrial transit petide in case in Table I, application no. 2, in column 6 in the same line as SEQ ID NO. 45757 is depicted, mitochondrial is mentioned; which is operable linke, too.

In another embodiment thereof the expression cassette does not comprise an additional transit peptide in case in Table I, application no. 2, in column 6 in the same line as SEQ ID NO. 45757 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 45795, or a homolog or fragment thereof, or a homolog thereof being depicted in Table I, application no. 2, column 8, in the same line as SEQ ID NO. 45795, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 45795 is depicted, plastidic is mentioned, or a mitochondrial transit petide in case in Table I, application no. 2, in column 6 in the same line as SEQ ID NO. 45795 is depicted, mitochondrial is mentioned; which is operable linke, too.

In another embodiment thereof the expression cassette does not comprise an additional transit peptide in case in Table I, application no. 2, in column 6 in the same line as SEQ ID NO. 45795 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 10811, or a homolog or fragment thereof, or a homolog thereof being depicted in Table I, application no. 2, column 8, in the same line as SEQ ID NO. 10811, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 10811 is depicted, plastidic is mentioned, or a mitochondrial transit petide in case in Table I, application no. 2, in column 6 in the same line as SEQ ID NO. 10811 is depicted, mitochondrial is mentioned; which is operable linke, too.

In another embodiment thereof the expression cassette does not comprise an additional transit peptide in case in Table I, application no. 2, in column 6 in the same line as SEQ ID NO. 10811 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 45897, or a homolog or fragment thereof, or a homolog thereof being depicted in Table I, application no. 2, column 8, in the same line as SEQ ID NO. 45897, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 45897 is depicted, plastidic is mentioned, or a mitochondrial transit petide in case in Table I, application no. 2, in column 6 in the same line as SEQ ID NO. 45897 is depicted, mitochondrial is mentioned; which is operable linke, too.

In another embodiment thereof the expression cassette does not comprise an additional transit peptide in case in Table I, application no. 2, in column 6 in the same line as SEQ ID NO. 45897 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 46405, or a homolog or fragment thereof, or a homolog thereof being depicted in Table I, application no. 2, column 8, in the same line as SEQ ID NO. 46405, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 46405 is depicted, plastidic is mentioned, or a mitochondrial transit petide in case in Table I, application no. 2, in column 6 in the same line as SEQ ID NO. 46405 is depicted, mitochondrial is mentioned; which is operable linke, too.

In another embodiment thereof the expression cassette does not comprise an additional transit peptide in case in Table I, application no. 2, in column 6 in the same line as SEQ ID NO. 46405 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 46515, or a homolog or fragment thereof, or a homolog thereof being depicted in Table I, application no. 2, column 8, in the same line as SEQ ID NO. 46515, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 46515 is depicted, plastidic is mentioned, or a mitochondrial transit petide in case in Table I, application no. 2, in column 6 in the same line as SEQ ID NO. 46515 is depicted, mitochondrial is mentioned; which is operable linke, too.

In another embodiment thereof the expression cassette does not comprise an additional transit peptide in case in Table I, application no. 2, in column 6 in the same line as SEQ ID NO. 46515 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 56576, or a homolog or fragment thereof, or a homolog thereof being depicted in Table I, application no. 2, column 8, in the same line as SEQ ID NO. 56576, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 56576 is depicted, plastidic is mentioned, or a mitochondrial transit petide in case in Table I, application no. 2, in column 6 in the same line as SEQ ID NO. 56576 is depicted, mitochondrial is mentioned; which is operable linke, too.

In another embodiment thereof the expression cassette does not comprise an additional transit peptide in case in Table I, application no. 2, in column 6 in the same line as SEQ ID NO. 56576 is depicted, non-targeted is mentioned.

In an embodiment the present invention relates to an expresssion cassette comprising a) a promoter, prefereably a promoter selected from the group consisting of Big35S, PCUbi, Super and USP, in particular USP; and b) the nucleic acid SEQ ID NO. 45022, or a homolog or fragment thereof, or a homolog thereof being depicted in Table I, application no. 2, column 8, in the same line as SEQ ID NO. 45022, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 45022 is depicted, plastidic is mentioned, or a mitochondrial transit petide in case in Table I, application no. 2, in column 6 in the same line as SEQ ID NO. 45022 is depicted, mitochondrial is mentioned; which is operable linke, too.

In another embodiment thereof the expression cassette does not comprise an additional transit peptide in case in Table I, application no. 2, in column 6 in the same line as SEQ ID NO. 45022 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 42559, or a homolog or fragment thereof, or a homolog thereof being depicted in Table I, application no. 2, column 8, in the same line as SEQ ID NO. 42559, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 42559 is depicted, plastidic is mentioned, or a mitochondrial transit petide in case in Table I, application no. 2, in column 6 in the same line as SEQ ID NO. 42559 is depicted, mitochondrial is mentioned; which is operable linke, too.

In another embodiment thereof the expression cassette does not comprise an additional transit peptide in case in Table I, application no. 2, in column 6 in the same line as SEQ ID NO. 42559 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 41499, or a homolog or fragment thereof, or a homolog thereof being depicted in Table I, application no. 2, column 8, in the same line as SEQ ID NO. 41499, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 41499 is depicted, plastidic is mentioned, or a mitochondrial transit petide in case in Table I, application no. 2, in column 6 in the same line as SEQ ID NO. 41499 is depicted, mitochondrial is mentioned; which is operable linke, too.

In another embodiment thereof the expression cassette does not comprise an additional transit peptide in case in Table I, application no. 2, in column 6 in the same line as SEQ ID NO. 41499 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 41732, or a homolog or fragment thereof, or a homolog thereof being depicted in Table I, application no. 2, column 8, in the same line as SEQ ID NO. 41732, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 41732 is depicted, plastidic is mentioned, or a mitochondrial transit petide in case in Table I, application no. 2, in column 6 in the same line as SEQ ID NO. 41732 is depicted, mitochondrial is mentioned; which is operable linke, too.

In another embodiment thereof the expression cassette does not comprise an additional transit peptide in case in Table I, application no. 2, in column 6 in the same line as SEQ ID NO. 41732 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 41797, or a homolog or fragment thereof, or a homolog thereof being depicted in Table I, application no. 2, column 8, in the same line as SEQ ID NO. 41797, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 41797 is depicted, plastidic is mentioned, or a mitochondrial transit petide in case in Table I, application no. 2, in column 6 in the same line as SEQ ID NO. 41797 is depicted, mitochondrial is mentioned; which is operable linke, too.

In another embodiment thereof the expression cassette does not comprise an additional transit peptide in case in Table I, application no. 2, in column 6 in the same line as SEQ ID NO. 41797 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 42046, or a homolog or fragment thereof, or a homolog thereof being depicted in Table I, application no. 2, column 8, in the same line as SEQ ID NO. 42046, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 42046 is depicted, plastidic is mentioned, or a mitochondrial transit petide in case in Table I, application no. 2, in column 6 in the same line as SEQ ID NO. 42046 is depicted, mitochondrial is mentioned; which is operable linke, too.

In another embodiment thereof the expression cassette does not comprise an additional transit peptide in case in Table I, application no. 2, in column 6 in the same line as SEQ ID NO. 42046 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 42471, or a homolog or fragment thereof, or a homolog thereof being depicted in Table I, application no. 2, column 8, in the same line as SEQ ID NO. 42471, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 42471 is depicted, plastidic is mentioned, or a mitochondrial transit petide in case in Table I, application no. 2, in column 6 in the same line as SEQ ID NO. 42471 is depicted, mitochondrial is mentioned; which is operable linke, too.

In another embodiment thereof the expression cassette does not comprise an additional transit peptide in case in Table I, application no. 2, in column 6 in the same line as SEQ ID NO. 42471 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 42477, or a homolog or fragment thereof, or a homolog thereof being depicted in Table I, application no. 2, column 8, in the same line as SEQ ID NO. 42477, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 42477 is depicted, plastidic is mentioned, or a mitochondrial transit petide in case in Table I, application no. 2, in column 6 in the same line as SEQ ID NO. 42477 is depicted, mitochondrial is mentioned; which is operable linke, too.

In another embodiment thereof the expression cassette does not comprise an additional transit peptide in case in Table I, application no. 2, in column 6 in the same line as SEQ ID NO. 42477 is depicted, non-targeted is mentioned.

In an embodiment the present invention relates to an expresssion cassette comprising a) a promoter, prefereably a promoter selected from the group consisting of Big35S, PCUbi, Super and USP, in particular USP; and b) the nucleic acid SEQ ID NO. 9492, or a homolog or fragment thereof, or a homolog thereof being depicted in Table I, application no. 2, column 8, in the same line as SEQ ID NO. 9492, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 9492 is depicted, plastidic is mentioned, or a mitochondrial transit petide in case in Table I, application no. 2, in column 6 in the same line as SEQ ID NO. 9492 is depicted, mitochondrial is mentioned; which is operable linke, too.

In another embodiment thereof the expression cassette does not comprise an additional transit peptide in case in Table I, application no. 2, in column 6 in the same line as SEQ ID NO. 9492 is depicted, non-targeted is mentioned.

In an embodiment the present invention relates to an expresssion cassette comprising a) a promoter, prefereably a promoter selected from the group consisting of Big35S, PCUbi, Super and USP, in particular USP; and b) the nucleic acid SEQ ID NO. 44223, or a homolog or fragment thereof, or a homolog thereof being depicted in Table I, application no. 2, column 8, in the same line as SEQ ID NO. 44223, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 44223 is depicted, plastidic is mentioned, or a mitochondrial transit petide in case in Table I, application no. 2, in column 6 in the same line as SEQ ID NO. 44223 is depicted, mitochondrial is mentioned; which is operable linke, too.

In another embodiment thereof the expression cassette does not comprise an additional transit peptide in case in Table I, application no. 2, in column 6 in the same line as SEQ ID NO. 44223 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 10104, or a homolog or fragment thereof, or a homolog thereof being depicted in Table I, application no. 2, column 8, in the same line as SEQ ID NO. 10104, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 10104 is depicted, plastidic is mentioned, or a mitochondrial transit petide in case in Table I, application no. 2, in column 6 in the same line as SEQ ID NO. 10104 is depicted, mitochondrial is mentioned; which is operable linke, too.

In another embodiment thereof the expression cassette does not comprise an additional transit peptide in case in Table I, application no. 2, in column 6 in the same line as SEQ ID NO. 10104 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 47026, or a homolog or fragment thereof, or a homolog thereof being depicted in Table I, application no. 2, column 8, in the same line as SEQ ID NO. 47026, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 47026 is depicted, plastidic is mentioned, or a mitochondrial transit petide in case in Table I, application no. 2, in column 6 in the same line as SEQ ID NO. 47026 is depicted, mitochondrial is mentioned; which is operable linke, too.

In another embodiment thereof the expression cassette does not comprise an additional transit peptide in case in Table I, application no. 2, in column 6 in the same line as SEQ ID NO. 47026 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 42579, or a homolog or fragment thereof, or a homolog thereof being depicted in Table I, application no. 2, column 8, in the same line as SEQ ID NO. 42579, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 42579 is depicted, plastidic is mentioned, or a mitochondrial transit petide in case in Table I, application no. 2, in column 6 in the same line as SEQ ID NO. 42579 is depicted, mitochondrial is mentioned; which is operable linke, too.

In another embodiment thereof the expression cassette does not comprise an additional transit peptide in case in Table I, application no. 2, in column 6 in the same line as SEQ ID NO. 42579 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 42592, or a homolog or fragment thereof, or a homolog thereof being depicted in Table I, application no. 2, column 8, in the same line as SEQ ID NO. 42592, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 42592 is depicted, plastidic is mentioned, or a mitochondrial transit petide in case in Table I, application no. 2, in column 6 in the same line as SEQ ID NO. 42592 is depicted, mitochondrial is mentioned; which is operable linke, too.

In another embodiment thereof the expression cassette does not comprise an additional transit peptide in case in Table I, application no. 2, in column 6 in the same line as SEQ ID NO. 42592 is depicted, non-targeted is mentioned.

In an embodiment the present invention relates to an expresssion cassette comprising a) a promoter, prefereably a promoter selected from the group consisting of Big35S, PCUbi, Super and USP, in particular USP; and b) the nucleic acid SEQ ID NO. 10172, or a homolog or fragment thereof, or a homolog thereof being depicted in Table I, application no. 2, column 8, in the same line as SEQ ID NO. 10172, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 10172 is depicted, plastidic is mentioned, or a mitochondrial transit petide in case in Table I, application no. 2, in column 6 in the same line as SEQ ID NO. 10172 is depicted, mitochondrial is mentioned; which is operable linke, too.

In another embodiment thereof the expression cassette does not comprise an additional transit peptide in case in Table I, application no. 2, in column 6 in the same line as SEQ ID NO. 10172 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 42600, or a homolog or fragment thereof, or a homolog thereof being depicted in Table I, application no. 2, column 8, in the same line as SEQ ID NO. 42600, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 42600 is depicted, plastidic is mentioned, or a mitochondrial transit petide in case in Table I, application no. 2, in column 6 in the same line as SEQ ID NO. 42600 is depicted, mitochondrial is mentioned; which is operable linke, too.

In another embodiment thereof the expression cassette does not comprise an additional transit peptide in case in Table I, application no. 2, in column 6 in the same line as SEQ ID NO. 42600 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 42931, or a homolog or fragment thereof, or a homolog thereof being depicted in Table I, application no. 2, column 8, in the same line as SEQ ID NO. 42931, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 42931 is depicted, plastidic is mentioned, or a mitochondrial transit petide in case in Table I, application no. 2, in column 6 in the same line as SEQ ID NO. 42931 is depicted, mitochondrial is mentioned; which is operable linke, too.

In another embodiment thereof the expression cassette does not comprise an additional transit peptide in case in Table I, application no. 2, in column 6 in the same line as SEQ ID NO. 42931 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 43248, or a homolog or fragment thereof, or a homolog thereof being depicted in Table I, application no. 2, column 8, in the same line as SEQ ID NO. 43248, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 43248 is depicted, plastidic is mentioned, or a mitochondrial transit petide in case in Table I, application no. 2, in column 6 in the same line as SEQ ID NO. 43248 is depicted, mitochondrial is mentioned; which is operable linke, too.

In another embodiment thereof the expression cassette does not comprise an additional transit peptide in case in Table I, application no. 2, in column 6 in the same line as SEQ ID NO. 43248 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 43800, or a homolog or fragment thereof, or a homolog thereof being depicted in Table I, application no. 2, column 8, in the same line as SEQ ID NO. 43800, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 43800 is depicted, plastidic is mentioned, or a mitochondrial transit petide in case in Table I, application no. 2, in column 6 in the same line as SEQ ID NO. 43800 is depicted, mitochondrial is mentioned; which is operable linke, too.

In another embodiment thereof the expression cassette does not comprise an additional transit peptide in case in Table I, application no. 2, in column 6 in the same line as SEQ ID NO. 43800 is depicted, non-targeted is mentioned.

In an embodiment the present invention relates to an expresssion cassette comprising a) a promoter, prefereably a promoter selected from the group consisting of Big35S, PCUbi, Super and USP, in particular USP; and b) the nucleic acid SEQ ID NO. 43839, or a homolog or fragment thereof, or a homolog thereof being depicted in Table I, application no. 2, column 8, in the same line as SEQ ID NO. 43839, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 43839 is depicted, plastidic is mentioned, or a mitochondrial transit petide in case in Table I, application no. 2, in column 6 in the same line as SEQ ID NO. 43839 is depicted, mitochondrial is mentioned; which is operable linke, too.

In another embodiment thereof the expression cassette does not comprise an additional transit peptide in case in Table I, application no. 2, in column 6 in the same line as SEQ ID NO. 43839 is depicted, non-targeted is mentioned.

In an embodiment the present invention relates to an expresssion cassette comprising a) a promoter, prefereably a promoter selected from the group consisting of Big35S, PCUbi, Super and USP, in particular Super; and b) the nucleic acid SEQ ID NO. 42502, or a homolog or fragment thereof, or a homolog thereof being depicted in Table I, application no. 2, column 8, in the same line as SEQ ID NO. 42502, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 42502 is depicted, plastidic is mentioned, or a mitochondrial transit petide in case in Table I, application no. 2, in column 6 in the same line as SEQ ID NO. 42502 is depicted, mitochondrial is mentioned; which is operable linke, too.

In another embodiment thereof the expression cassette does not comprise an additional transit peptide in case in Table I, application no. 2, in column 6 in the same line as SEQ ID NO. 42502 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 54804, or a homolog or fragment thereof, or a homolog thereof being depicted in Table I, application no. 2, column 8, in the same line as SEQ ID NO. 54804, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 54804 is depicted, plastidic is mentioned, or a mitochondrial transit petide in case in Table I, application no. 2, in column 6 in the same line as SEQ ID NO. 54804 is depicted, mitochondrial is mentioned; which is operable linke, too.

In another embodiment thereof the expression cassette does not comprise an additional transit peptide in case in Table I, application no. 2, in column 6 in the same line as SEQ ID NO. 54804 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 46751, or a homolog or fragment thereof, or a homolog thereof being depicted in Table I, application no. 2, column 8, in the same line as SEQ ID NO. 46751, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 46751 is depicted, plastidic is mentioned, or a mitochondrial transit petide in case in Table I, application no. 2, in column 6 in the same line as SEQ ID NO. 46751 is depicted, mitochondrial is mentioned; which is operable linke, too.

In another embodiment thereof the expression cassette does not comprise an additional transit peptide in case in Table I, application no. 2, in column 6 in the same line as SEQ ID NO. 46751 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 52660, or a homolog or fragment thereof, or a homolog thereof being depicted in Table I, application no. 2, column 8, in the same line as SEQ ID NO. 52660, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 52660 is depicted, plastidic is mentioned, or a mitochondrial transit petide in case in Table I, application no. 2, in column 6 in the same line as SEQ ID NO. 52660 is depicted, mitochondrial is mentioned; which is operable linke, too.

In another embodiment thereof the expression cassette does not comprise an additional transit peptide in case in Table I, application no. 2, in column 6 in the same line as SEQ ID NO. 52660 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 53189, or a homolog or fragment thereof, or a homolog thereof being depicted in Table I, application no. 2, column 8, in the same line as SEQ ID NO. 53189, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 53189 is depicted, plastidic is mentioned, or a mitochondrial transit petide in case in Table I, application no. 2, in column 6 in the same line as SEQ ID NO. 53189 is depicted, mitochondrial is mentioned; which is operable linke, too.

In another embodiment thereof the expression cassette does not comprise an additional transit peptide in case in Table I, application no. 2, in column 6 in the same line as SEQ ID NO. 53189 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 53456, or a homolog or fragment thereof, or a homolog thereof being depicted in Table I, application no. 2, column 8, in the same line as SEQ ID NO. 53456, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 53456 is depicted, plastidic is mentioned, or a mitochondrial transit petide in case in Table I, application no. 2, in column 6 in the same line as SEQ ID NO. 53456 is depicted, mitochondrial is mentioned; which is operable linke, too.

In another embodiment thereof the expression cassette does not comprise an additional transit peptide in case in Table I, application no. 2, in column 6 in the same line as SEQ ID NO. 53456 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 53608, or a homolog or fragment thereof, or a homolog thereof being depicted in Table I, application no. 2, column 8, in the same line as SEQ ID NO. 53608, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 53608 is depicted, plastidic is mentioned, or a mitochondrial transit petide in case in Table I, application no. 2, in column 6 in the same line as SEQ ID NO. 53608 is depicted, mitochondrial is mentioned; which is operable linke, too.

In another embodiment thereof the expression cassette does not comprise an additional transit peptide in case in Table I, application no. 2, in column 6 in the same line as SEQ ID NO. 53608 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 53878, or a homolog or fragment thereof, or a homolog thereof being depicted in Table I, application no. 2, column 8, in the same line as SEQ ID NO. 53878, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 53878 is depicted, plastidic is mentioned, or a mitochondrial transit petide in case in Table I, application no. 2, in column 6 in the same line as SEQ ID NO. 53878 is depicted, mitochondrial is mentioned; which is operable linke, too.

In another embodiment thereof the expression cassette does not comprise an additional transit peptide in case in Table I, application no. 2, in column 6 in the same line as SEQ ID NO. 53878 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 54337, or a homolog or fragment thereof, or a homolog thereof being depicted in Table I, application no. 2, column 8, in the same line as SEQ ID NO. 54337, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 54337 is depicted, plastidic is mentioned, or a mitochondrial transit petide in case in Table I, application no. 2, in column 6 in the same line as SEQ ID NO. 54337 is depicted, mitochondrial is mentioned; which is operable linke, too.

In another embodiment thereof the expression cassette does not comprise an additional transit peptide in case in Table I, application no. 2, in column 6 in the same line as SEQ ID NO. 54337 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 52364, or a homolog or fragment thereof, or a homolog thereof being depicted in Table I, application no. 2, column 8, in the same line as SEQ ID NO. 52364, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 52364 is depicted, plastidic is mentioned, or a mitochondrial transit petide in case in Table I, application no. 2, in column 6 in the same line as SEQ ID NO. 52364 is depicted, mitochondrial is mentioned; which is operable linke, too.

In another embodiment thereof the expression cassette does not comprise an additional transit peptide in case in Table I, application no. 2, in column 6 in the same line as SEQ ID NO. 52364 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 11423, or a homolog or fragment thereof, or a homolog thereof being depicted in Table I, application no. 2, column 8, in the same line as SEQ ID NO. 11423, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 11423 is depicted, plastidic is mentioned, or a mitochondrial transit petide in case in Table I, application no. 2, in column 6 in the same line as SEQ ID NO. 11423 is depicted, mitochondrial is mentioned; which is operable linke, too.

In another embodiment thereof the expression cassette does not comprise an additional transit peptide in case in Table I, application no. 2, in column 6 in the same line as SEQ ID NO. 11423 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 52246, or a homolog or fragment thereof, or a homolog thereof being depicted in Table I, application no. 2, column 8, in the same line as SEQ ID NO. 52246, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 52246 is depicted, plastidic is mentioned, or a mitochondrial transit petide in case in Table I, application no. 2, in column 6 in the same line as SEQ ID NO. 52246 is depicted, mitochondrial is mentioned; which is operable linke, too.

In another embodiment thereof the expression cassette does not comprise an additional transit peptide in case in Table I, application no. 2, in column 6 in the same line as SEQ ID NO. 52246 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 54897, or a homolog or fragment thereof, or a homolog thereof being depicted in Table I, application no. 2, column 8, in the same line as SEQ ID NO. 54897, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 54897 is depicted, plastidic is mentioned, or a mitochondrial transit petide in case in Table I, application no. 2, in column 6 in the same line as SEQ ID NO. 54897 is depicted, mitochondrial is mentioned; which is operable linke, too.

In another embodiment thereof the expression cassette does not comprise an additional transit peptide in case in Table I, application no. 2, in column 6 in the same line as SEQ ID NO. 54897 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 55063, or a homolog or fragment thereof, or a homolog thereof being depicted in Table I, application no. 2, column 8, in the same line as SEQ ID NO. 55063, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 55063 is depicted, plastidic is mentioned, or a mitochondrial transit petide in case in Table I, application no. 2, in column 6 in the same line as SEQ ID NO. 55063 is depicted, mitochondrial is mentioned; which is operable linke, too.

In another embodiment thereof the expression cassette does not comprise an additional transit peptide in case in Table I, application no. 2, in column 6 in the same line as SEQ ID NO. 55063 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 55379, or a homolog or fragment thereof, or a homolog thereof being depicted in Table I, application no. 2, column 8, in the same line as SEQ ID NO. 55379, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 55379 is depicted, plastidic is mentioned, or a mitochondrial transit petide in case in Table I, application no. 2, in column 6 in the same line as SEQ ID NO. 55379 is depicted, mitochondrial is mentioned; which is operable linke, too.

In another embodiment thereof the expression cassette does not comprise an additional transit peptide in case in Table I, application no. 2, in column 6 in the same line as SEQ ID NO. 55379 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 11471, or a homolog or fragment thereof, or a homolog thereof being depicted in Table I, application no. 2, column 8, in the same line as SEQ ID NO. 11471, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 11471 is depicted, plastidic is mentioned, or a mitochondrial transit petide in case in Table I, application no. 2, in column 6 in the same line as SEQ ID NO. 11471 is depicted, mitochondrial is mentioned; which is operable linke, too.

In another embodiment thereof the expression cassette does not comprise an additional transit peptide in case in Table I, application no. 2, in column 6 in the same line as SEQ ID NO. 11471 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 55385, or a homolog or fragment thereof, or a homolog thereof being depicted in Table I, application no. 2, column 8, in the same line as SEQ ID NO. 55385, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 55385 is depicted, plastidic is mentioned, or a mitochondrial transit petide in case in Table I, application no. 2, in column 6 in the same line as SEQ ID NO. 55385 is depicted, mitochondrial is mentioned; which is operable linke, too.

In another embodiment thereof the expression cassette does not comprise an additional transit peptide in case in Table I, application no. 2, in column 6 in the same line as SEQ ID NO. 55385 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 55771, or a homolog or fragment thereof, or a homolog thereof being depicted in Table I, application no. 2, column 8, in the same line as SEQ ID NO. 55771, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 55771 is depicted, plastidic is mentioned, or a mitochondrial transit petide in case in Table I, application no. 2, in column 6 in the same line as SEQ ID NO. 55771 is depicted, mitochondrial is mentioned; which is operable linke, too.

In another embodiment thereof the expression cassette does not comprise an additional transit peptide in case in Table I, application no. 2, in column 6 in the same line as SEQ ID NO. 55771 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 55978, or a homolog or fragment thereof, or a homolog thereof being depicted in Table I, application no. 2, column 8, in the same line as SEQ ID NO. 55978, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 55978 is depicted, plastidic is mentioned, or a mitochondrial transit petide in case in Table I, application no. 2, in column 6 in the same line as SEQ ID NO. 55978 is depicted, mitochondrial is mentioned; which is operable linke, too.

In another embodiment thereof the expression cassette does not comprise an additional transit peptide in case in Table I, application no. 2, in column 6 in the same line as SEQ ID NO. 55978 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 68413, or a homolog or fragment thereof, or a homolog thereof being depicted in Table I, application no. 2, column 8, in the same line as SEQ ID NO. 68413, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 68413 is depicted, plastidic is mentioned, or a mitochondrial transit petide in case in Table I, application no. 2, in column 6 in the same line as SEQ ID NO. 68413 is depicted, mitochondrial is mentioned; which is operable linke, too.

In another embodiment thereof the expression cassette does not comprise an additional transit peptide in case in Table I, application no. 2, in column 6 in the same line as SEQ ID NO. 68413 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 54452, or a homolog or fragment thereof, or a homolog thereof being depicted in Table I, application no. 2, column 8, in the same line as SEQ ID NO. 54452, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 54452 is depicted, plastidic is mentioned, or a mitochondrial transit petide in case in Table I, application no. 2, in column 6 in the same line as SEQ ID NO. 54452 is depicted, mitochondrial is mentioned; which is operable linke, too.

In another embodiment thereof the expression cassette does not comprise an additional transit peptide in case in Table I, application no. 2, in column 6 in the same line as SEQ ID NO. 54452 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 49828, or a homolog or fragment thereof, or a homolog thereof being depicted in Table I, application no. 2, column 8, in the same line as SEQ ID NO. 49828, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 49828 is depicted, plastidic is mentioned, or a mitochondrial transit petide in case in Table I, application no. 2, in column 6 in the same line as SEQ ID NO. 49828 is depicted, mitochondrial is mentioned; which is operable linke, too.

In another embodiment thereof the expression cassette does not comprise an additional transit peptide in case in Table I, application no. 2, in column 6 in the same line as SEQ ID NO. 49828 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 47076, or a homolog or fragment thereof, or a homolog thereof being depicted in Table I, application no. 2, column 8, in the same line as SEQ ID NO. 47076, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 47076 is depicted, plastidic is mentioned, or a mitochondrial transit petide in case in Table I, application no. 2, in column 6 in the same line as SEQ ID NO. 47076 is depicted, mitochondrial is mentioned; which is operable linke, too.

In another embodiment thereof the expression cassette does not comprise an additional transit peptide in case in Table I, application no. 2, in column 6 in the same line as SEQ ID NO. 47076 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 47105, or a homolog or fragment thereof, or a homolog thereof being depicted in Table I, application no. 2, column 8, in the same line as SEQ ID NO. 47105, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 47105 is depicted, plastidic is mentioned, or a mitochondrial transit petide in case in Table I, application no. 2, in column 6 in the same line as SEQ ID NO. 47105 is depicted, mitochondrial is mentioned; which is operable linke, too.

In another embodiment thereof the expression cassette does not comprise an additional transit peptide in case in Table I, application no. 2, in column 6 in the same line as SEQ ID NO. 47105 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 47159, or a homolog or fragment thereof, or a homolog thereof being depicted in Table I, application no. 2, column 8, in the same line as SEQ ID NO. 47159, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 47159 is depicted, plastidic is mentioned, or a mitochondrial transit petide in case in Table I, application no. 2, in column 6 in the same line as SEQ ID NO. 47159 is depicted, mitochondrial is mentioned; which is operable linke, too.

In another embodiment thereof the expression cassette does not comprise an additional transit peptide in case in Table I, application no. 2, in column 6 in the same line as SEQ ID NO. 47159 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 47526, or a homolog or fragment thereof, or a homolog thereof being depicted in Table I, application no. 2, column 8, in the same line as SEQ ID NO. 47526, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 47526 is depicted, plastidic is mentioned, or a mitochondrial transit petide in case in Table I, application no. 2, in column 6 in the same line as SEQ ID NO. 47526 is depicted, mitochondrial is mentioned; which is operable linke, too.

In another embodiment thereof the expression cassette does not comprise an additional transit peptide in case in Table I, application no. 2, in column 6 in the same line as SEQ ID NO. 47526 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 47566, or a homolog or fragment thereof, or a homolog thereof being depicted in Table I, application no. 2, column 8, in the same line as SEQ ID NO. 47566, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 47566 is depicted, plastidic is mentioned, or a mitochondrial transit petide in case in Table I, application no. 2, in column 6 in the same line as SEQ ID NO. 47566 is depicted, mitochondrial is mentioned; which is operable linke, too.

In another embodiment thereof the expression cassette does not comprise an additional transit peptide in case in Table I, application no. 2, in column 6 in the same line as SEQ ID NO. 47566 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 48138, or a homolog or fragment thereof, or a homolog thereof being depicted in Table I, application no. 2, column 8, in the same line as SEQ ID NO. 48138, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 48138 is depicted, plastidic is mentioned, or a mitochondrial transit petide in case in Table I, application no. 2, in column 6 in the same line as SEQ ID NO. 48138 is depicted, mitochondrial is mentioned; which is operable linke, too.

In another embodiment thereof the expression cassette does not comprise an additional transit peptide in case in Table I, application no. 2, in column 6 in the same line as SEQ ID NO. 48138 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 49143, or a homolog or fragment thereof, or a homolog thereof being depicted in Table I, application no. 2, column 8, in the same line as SEQ ID NO. 49143, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 49143 is depicted, plastidic is mentioned, or a mitochondrial transit petide in case in Table I, application no. 2, in column 6 in the same line as SEQ ID NO. 49143 is depicted, mitochondrial is mentioned; which is operable linke, too.

In another embodiment thereof the expression cassette does not comprise an additional transit peptide in case in Table I, application no. 2, in column 6 in the same line as SEQ ID NO. 49143 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi and b) the nucleic acid SEQ ID NO. 52634, or a homolog or fragment thereof, or a homolog thereof being depicted in Table I, application no. 2, column 8, in the same line as SEQ ID NO. 52634, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 52634 is depicted, plastidic is mentioned, or a mitochondrial transit petide in case in Table I, application no. 2, in column 6 in the same line as SEQ ID NO. 52634 is depicted, mitochondrial is mentioned; which is operable linke, too.

In another embodiment thereof the expression cassette does not comprise an additional transit peptide in case in Table I, application no. 2, in column 6 in the same line as SEQ ID NO. 52634 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 49800, or a homolog or fragment thereof, or a homolog thereof being depicted in Table I, application no. 2, column 8, in the same line as SEQ ID NO. 49800, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 49800 is depicted, plastidic is mentioned, or a mitochondrial transit petide in case in Table I, application no. 2, in column 6 in the same line as SEQ ID NO. 49800 is depicted, mitochondrial is mentioned; which is operable linke, too.

In another embodiment thereof the expression cassette does not comprise an additional transit peptide in case in Table I, application no. 2, in column 6 in the same line as SEQ ID NO. 49800 is depicted, non-targeted is mentioned.

In an embodiment the present invention relates to an expresssion cassette comprising a) a promoter, prefereably a promoter selected from the group consisting of Big35S, PCUbi, Super and USP, in particularPCUbi; and b) the nucleic acid SEQ ID NO. 56514, or a homolog or fragment thereof, or a homolog thereof being depicted in Table I, application no. 2, column 8, in the same line as SEQ ID NO. 56514, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 56514 is depicted, plastidic is mentioned, or a mitochondrial transit petide in case in Table I, application no. 2, in column 6 in the same line as SEQ ID NO. 56514 is depicted, mitochondrial is mentioned; which is operable linke, too.

In another embodiment thereof the expression cassette does not comprise an additional transit peptide in case in Table I, application no. 2, in column 6 in the same line as SEQ ID NO. 56514 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 50070, or a homolog or fragment thereof, or a homolog thereof being depicted in Table I, application no. 2, column 8, in the same line as SEQ ID NO. 50070, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 50070 is depicted, plastidic is mentioned, or a mitochondrial transit petide in case in Table I, application no. 2, in column 6 in the same line as SEQ ID NO. 50070 is depicted, mitochondrial is mentioned; which is operable linke, too.

In another embodiment thereof the expression cassette does not comprise an additional transit peptide in case in Table I, application no. 2, in column 6 in the same line as SEQ ID NO. 50070 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 50104, or a homolog or fragment thereof, or a homolog thereof being depicted in Table I, application no. 2, column 8, in the same line as SEQ ID NO. 50104, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 50104 is depicted, plastidic is mentioned, or a mitochondrial transit petide in case in Table I, application no. 2, in column 6 in the same line as SEQ ID NO. 50104 is depicted, mitochondrial is mentioned; which is operable linke, too.

In another embodiment thereof the expression cassette does not comprise an additional transit peptide in case in Table I, application no. 2, in column 6 in the same line as SEQ ID NO. 50104 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 50339, or a homolog or fragment thereof, or a homolog thereof being depicted in Table I, application no. 2, column 8, in the same line as SEQ ID NO. 50339, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 50339 is depicted, plastidic is mentioned, or a mitochondrial transit petide in case in Table I, application no. 2, in column 6 in the same line as SEQ ID NO. 50339 is depicted, mitochondrial is mentioned; which is operable linke, too.

In another embodiment thereof the expression cassette does not comprise an additional transit peptide in case in Table I, application no. 2, in column 6 in the same line as SEQ ID NO. 50339 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 50713, or a homolog or fragment thereof, or a homolog thereof being depicted in Table I, application no. 2, column 8, in the same line as SEQ ID NO. 50713, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 50713 is depicted, plastidic is mentioned, or a mitochondrial transit petide in case in Table I, application no. 2, in column 6 in the same line as SEQ ID NO. 50713 is depicted, mitochondrial is mentioned; which is operable linke, too.

In another embodiment thereof the expression cassette does not comprise an additional transit peptide in case in Table I, application no. 2, in column 6 in the same line as SEQ ID NO. 50713 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 50950, or a homolog or fragment thereof, or a homolog thereof being depicted in Table I, application no. 2, column 8, in the same line as SEQ ID NO. 50950, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 50950 is depicted, plastidic is mentioned, or a mitochondrial transit petide in case in Table I, application no. 2, in column 6 in the same line as SEQ ID NO. 50950 is depicted, mitochondrial is mentioned; which is operable linke, too.

In another embodiment thereof the expression cassette does not comprise an additional transit peptide in case in Table I, application no. 2, in column 6 in the same line as SEQ ID NO. 50950 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 51198, or a homolog or fragment thereof, or a homolog thereof being depicted in Table I, application no. 2, column 8, in the same line as SEQ ID NO. 51198, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 51198 is depicted, plastidic is mentioned, or a mitochondrial transit petide in case in Table I, application no. 2, in column 6 in the same line as SEQ ID NO. 51198 is depicted, mitochondrial is mentioned; which is operable linke, too.

In another embodiment thereof the expression cassette does not comprise an additional transit peptide in case in Table I, application no. 2, in column 6 in the same line as SEQ ID NO. 51198 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 51268, or a homolog or fragment thereof, or a homolog thereof being depicted in Table I, application no. 2, column 8, in the same line as SEQ ID NO. 51268, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 51268 is depicted, plastidic is mentioned, or a mitochondrial transit petide in case in Table I, application no. 2, in column 6 in the same line as SEQ ID NO. 51268 is depicted, mitochondrial is mentioned; which is operable linke, too.

In another embodiment thereof the expression cassette does not comprise an additional transit peptide in case in Table I, application no. 2, in column 6 in the same line as SEQ ID NO. 51268 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 51632, or a homolog or fragment thereof, or a homolog thereof being depicted in Table I, application no. 2, column 8, in the same line as SEQ ID NO. 51632, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 51632 is depicted, plastidic is mentioned, or a mitochondrial transit petide in case in Table I, application no. 2, in column 6 in the same line as SEQ ID NO. 51632 is depicted, mitochondrial is mentioned; which is operable linke, too.

In another embodiment thereof the expression cassette does not comprise an additional transit peptide in case in Table I, application no. 2, in column 6 in the same line as SEQ ID NO. 51632 is depicted, non-targeted is mentioned.

In an embodiment the present invention relates to an expresssion cassette comprising a) a promoter, prefereably a promoter selected from the group consisting of Big35S, PCUbi, Super and USP, in particular PCUbi; and b) the nucleic acid SEQ ID NO. 49342, or a homolog or fragment thereof, or a homolog thereof being depicted in Table I, application no. 2, column 8, in the same line as SEQ ID NO. 49342, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 2, in column 6 in the same line as SEQ ID NO. 49342 is depicted, plastidic is mentioned, or a mitochondrial transit petide in case in Table I, application no. 2, in column 6 in the same line as SEQ ID NO. 49342 is depicted, mitochondrial is mentioned; which is operable linke, too.

In another embodiment thereof the expression cassette does not comprise an additional transit peptide in case in Table I, application no. 2, in column 6 in the same line as SEQ ID NO. 49342 is depicted, non-targeted is mentioned.

[0165.1.1.2] to [0170.1.1.2] for the disclosure of these paragraphs see [0165.1.1.1] to [0170.1.1.1] above.

The invention further provides a vector, comprising a nucleic acid molecule comprising 1) a nucleic acid molecule selected from the group consisting of:
(a) a nucleic acid molecule encoding a polypeptide shown in Table II, column 5 or 8, application no. 2, preferably shown in Table II A, application no. 2, in column 5, or in Table II A, application no. 2, column 8, or in Table II B, application no. 2, column 8, or a homolog or a fragment thereof;

(b) a nucleic acid molecule shown in Table I, application no. 2, column 5 or 8, preferably shown in Table I A, application no. 2, in column 5, or in Table I A, application no. 2, column 8, or in Table I B, application no. 2, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 2, preferably shown in Table II A, application no. 2, in column 5, or in Table II A, application no. 2, column 8, or in Table II B, application no.,2, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 2, preferably shown in Table I A, application no. 2, in column 5, or in Table I A, application no. 2, column 8, or in Table I B, application no. 2,column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 2;
(i) a nucleic acid molecule encoding a polypeptide having the activity represented by a protein as depicted in column 5 of Table II, application no. 2;
(j) a nucleic acid molecule comprising a polynucleotide which is obtained by amplifying a cDNA library or a genomic library using the primers in Table III, application no. 2, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
   or a nucleic acid molecule comprising a sequence which is complementary thereto.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 2.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 2, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 2.

In another preferred embodiment thereof said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 2, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 2, in the same line as of the respective nucleic acid molecule according to 1) “plastidic” is depicted, or encoding a mitochondrial transit peptide, especially in case in column 6 of Table I, application no. 2, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 2, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 2, in the same line as of the respective nucleic acid molecule according to 1) “plastidic” is depicted, or encoding a mitochondrial transit peptide, especially in case in column 6 of Table I, application no. 2, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP, which are operable linked; a nucleic acid molecule encoding a transit peptide, especially in case in column 6 of Table I, application no. 2, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 2, in the same line as of the respective nucleic acid molecule according to 1) “plastidic” is depicted, or encoding a mitochondrial transit peptide, especially in case in column 6 of Table I, application no. 2, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 2 (in the same line).

In another embodiment thereof said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP, which are operatively linked; a nucleic acid molecule encoding a transit peptide, especially in case in column 6 of Table I, application no. 2, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 2.

In another embodiment thereof said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 2, in the same line as of the respective nucleic acid molecule according to 1) “plastidic” is depicted, or encoding a mitochondrial transit peptide, especially in case in column 6 of Table I, application no. 2, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP, which are operatively linked; a nucleic acid molecule encoding a transit peptide, especially in case in column 6 of Table I, application no. 2, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 2, in the same line as of the respective nucleic acid molecule according to 1) “plastidic” is depicted, or encoding a mitochondrial transit peptide, especially in case in column 6 of Table I, application no. 2, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 2 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP, which are operatively linked; a nucleic acid molecule encoding a transit peptide, especially in case in column 6 of Table I, application no. 2, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 2 (in the same line) and confers a generation or an increase of the respective fine chemical in a non-human organism or a part thereof as compared to a corresponding wild-type non-human organism or part thereof.

In a prefered embodiment of said vector said nucleic acid molecule according to 1 (a), (b), (c), (d), (e), (f), (g), (h), (i), (j) or (k), said promoter and optionally said nucleic acid molecule encoding a transit peptide are encompassed in an expression cassette. Accordingly in an embodiment the invention provides a vector comprising an expression casssette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

The invention further provides a vector comprising a nucleic acid molecule comprising 1) a nucleic acid molecule selected from the group consisting of:
(a) a nucleic acid molecule encoding a polypeptide shown in Table II, column 5 or 8, application no. 2, preferably shown in Table II A, application no. 2, in column 5, or in Table II A, application no. 2, column 8, or in Table II B, application no. 2, column 8, or a homolog or a fragment thereof;
(b) a nucleic acid molecule shown in Table I, application no. 2, column 5 or 8, preferably shown in Table I A, application no. 2, in column 5, or in Table I A, application no. 2, column 8, or in Table I B, application no. 2, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 2, preferably shown in Table II A, application no. 2, in column 5, or in Table II A, application no. 2, column 8, or in Table II B, application no. 2, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 2, preferably shown in Table I A, application no. 2, in column 5, or in Table I A, application no. 2, column 8, or in Table I B, application no. 2, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 2;
(i) a nucleic acid molecule encoding a polypeptide having the activity represented by a protein as depicted in column 5 of Table II, application no. 2;
(j) a nucleic acid molecule comprising a polynucleotide which is obtained by amplifying a cDNA library or a genomic library using the primers in Table III, application no. 2, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
   or a nucleic acid molecule comprising a sequence which is complementary thereto;
   whereby, preferably, the nucleic acid molecule according to 1 (a), (b), (c), (d), (e), (f), (g), (h), (i), (j) or (k) distinguishes over the sequence depicted in Table I A and/or I B, application no. 2, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 2, column 5 or 8, or the coding region thereof, respectively. In another embodiment, the nucleic acid molecule of the present invention, preferably the coding region thereof, is 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 2, column 5 or 8, or the coding region thereof, respectively. In a further embodiment the nucleic acid molecule does not encode the polypeptide sequence shown in Table II A and/or II B, application no. 2, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 2, column 5 or 8 and does not encode a protein of the sequence shown in Table II A and/or II B, application no. 2, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 2, column 5 and 8, or the coding region thereof. In a further embodiment, the protein of the present invention is at least 30%,40%, 50%, 60%, 70%, 80%, 90%, 95% identical to protein sequence depicted in Table II A and/or II B, application no. 2, column 5 or 8 but less than 100%, preferably less than 99.999%, 99.99% or 99.9%, more preferably less than 99%, 985, 97%, 96% or 95% identical to the sequence shown in Table II A and/or II B, application no. 2, column 5 or 8.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 2.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of

Table II, application no. 2, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 2.

In another preferred embodiment thereof said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 2, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 2, in the same line as of the respective nucleic acid molecule according to 1) “plastidic” is depicted, or encoding a mitochondrial transit peptide, especially in case in column 6 of Table I, application no. 2, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 2, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 2, in the same line as of the respective nucleic acid molecule according to 1) “plastidic” is depicted, or encoding a mitochondrial transit peptide, especially in case in column 6 of Table I, application no. 2, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP, which are operatively linked; a nucleic acid molecule encoding a transit peptide, especially in case in column 6 of Table I, application no. 2, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 2, in the same line as of the respective nucleic acid molecule according to 1) “plastidic” is depicted, or encoding a mitochondrial transit peptide, especially in case in column 6 of Table I, application no. 2, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 2.

In another embodiment thereof said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP, which are operatively linked; a nucleic acid molecule encoding a transit peptide, especially in case in column 6 of Table I, application no. 2, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 2.

In another embodiment thereof said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 2, in the same line as of the respective nucleic acid molecule according to 1) “plastidic” is depicted, or encoding a mitochondrial transit peptide, especially in case in column 6 of Table I, application no. 2, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP, which are operatively linked; a nucleic acid molecule encoding a transit peptide, especially in case in column 6 of Table I, application no. 2, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 2, in the same line as of the respective nucleic acid molecule according to 1) “plastidic” is depicted, or encoding a mitochondrial transit peptide, especially in case in column 6 of Table I, application no. 2, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 2 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP, which are operatively linked; a nucleic acid molecule encoding a transit peptide, especially in case in column 6 of Table I, application no. 2, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 2 and confers a generation or an increase of the respective fine chemical in a non-human organism or a part thereof as compared to a corresponding wild-type non-human organism or part thereof.

[0181.1.1.2] to [0209.1.1.2] for the disclosure of these paragraphs see [0181.1.1.1] to [0209.1.1.1] above.

The genes of the invention, coding for an activity selected from the group consisting of 2,3-dihydroxy-2,3-d ihyd rophenylpropionatedehyd rogenase, 2-oxoglutarate dehyd rogenase E1 subunit, 30S ribosomal protein, 3-deoxy-7-phosphoheptulonate synthase, 3-phosphoglycerate dehydrogenase, 47266012-protein, 49747384_SOYBEAN-protein, 4-alphaglucanotransferase, ABC transporter permease protein, acetyl CoA carboxylase, acetyltransferase, acid shock protein, aconitate hydratase, acyl-CoA dehydrogenase, acyl-CoA synthase, acyltransferase, adenylosuccinate lyase, adenylylsulfate kinase, alanine dehydrogenase, aldehyde dehydrogenase, allantoicase, allantoinase, amine oxidase, amino acid ABC transporter permease protein, amino acid acetyltransferase, aminotransferase, ankyrin repeat family protein, anthranilate synthase component II, arginine decarboxylase, arginine exporter protein, asparaginase, aspartase, aspartate aminotransferase, At1g17440-protein, At1g19800-protein, At1 g29350-protein, At1 g47380-protein, At1g67340-protein, At4g32480-protein, At5g16650-protein, ATP synthase subunit beta, ATP-binding component of a transport system, ATP-dependent RNA helicase, AX653549-protein, AY087308-protein, b0456-protein, b0518-protein, b1003-protein, b1024-protein, b1108-protein, b1137-protein, b1163-protein, b1259-protein, b1280-protein, b1330-protein, b1445-protein, b1522-protein, b1898-protein, b2107-protein, b2121-protein, b2360-protein, b2399-protein, b2474-protein, b2513-protein, b2548-protein, b2613-protein, b2673-protein, b2812-protein, b2846-protein, b2909-protein, b2936-protein, b2999-protein, b3121-protein, b3151-protein, b3346-protein, b3410-protein, B3427-protein, b3509-protein, b3814-protein, b3817-protein, b3989-protein, b4029-protein, b4121-protein, beta-galactosidase, beta-hydroxylase, bifunctional purine biosynthesis protein, branched-chain amino acid ABC transporter permease protein, branched-chain amino acid permease, calcium-dependent protein kinase, carbohydrate kinase, carbon dioxide concentrating mechanism protein, cation/acetate symporter, cation-transporting ATPase, CBL-interacting protein kinase, CCAAT-binding transcription factor, CDP-diacylglycerol-glycerol-3-phosphate 3-phosphatidyltransferase, CDP-diglyceride synthetase, cell division control protein, cell division protein,

Chaperone protein CIpB, circadian clock protein, coproporphyrinogen III oxidase, coproporphyrinogen oxidase, CTP synthase, CTP synthetase, cullin, cyclin D, cysteine synthase A, delta-1-pyrroline 5-carboxylase synthetase, dihydrolipoamide acetyltransferase, dihydrolipoamide dehydrogenase, DNA binding protein, DNA helicase II, DNA helicase IV, DNA mismatch repair protein, DnaJ-like chaperone, electron transport complex protein, elongation factor Tu, enoyl-CoA hydratase, enterobacterial common antigen polymerase, ethanolamine utilization protein, eukaryotic translation initiation factor 5, exopolyphosphatase, fatty acid desaturase, flavodoxin, fumarylacetoacetate hydrolase, gamma-glutamyltranspeptidase, geranylgeranyl pyrophosphate synthase, gibberellin 20-oxidase, Glu/Leu/Phe/Val dehydrogenase, gluconate transport system permease 3, glucose dehydrogenase, glucose-1-phosphate cytidylyltransferase, glucose-6-phosphate 1-dehydrogenase, glutamate-ammonia-ligase, glutamine amidotransferase, glutamine synthetase, glutaminyl-tRNA synthetase, glutaredoxin, glutathione S-transferase, glycerol dehydrogenase, glycerol-3-phosphate dehydrogenase, glycogen (starch) synthase, glycogen synthase, glycogenin, glycoprotease, glycosidase, glycosyl transferase, GM02LC11114-protein, GM02LC17485-protein, GM02LC46-protein, GM02LC5744-protein, H/ACA ribonucleoprotein complex subunit 3, harpin-induced family protein, heat shock protein, heat shock transcription factor, HesB/YadR/YfhF family protein, histone H2A, homocitrate synthase, hydrolase, integral membrane transporter family protein, iron(III) dicitrate-binding protein, isochorismate synthase, isocitrate dehydrogenase, isopentenyl diphosphate isomerase, L-asparaginase, L-aspartate oxidase, Leucyl/phenylalanyl-tRNA-protein transferase, lipoprotein precursor, L-ribulose-5-phosphate 4-epimerase, lysyl-tRNA synthetase, major facilitator superfamily transporter protein, malate dehydrogenase, malic enzyme, malonyl CoA-acyl carrier protein transacylase, membrane protein, membrane transport protein, metal-dependent hydrolase, methylglyoxal synthase, methyltransferase, mitochondrial processing protease, molecular chaperone portein, monothiol glutaredoxin, monthiol glutaredoxin, multiple antibiotic resistance protein, murein transglycosylase, N-acetyl-gamma-glutamyl-phosphate reductase, NAD(P)H-quinone oxidoreductase subunit, NADH dehydrogenase I chain I, nitrate/nitrite transport protein, ornithine carbamoyltransferase, oxidoreductase, oxidoreductase subunit, oxireductase, permease protein of phosphate ABC transporter, peroxisome assembly protein, phosphatidylinositol 3- and 4-kinase family protein, phosphoadenosine phosphosulfate reductase, phosphoanhydride phosphorylase, phosphopantetheine adenylyltransferase, phosphopantothenoylcysteine decarboxylase, phosphopantothenoylcysteinesynthetase/decarboxylase, phosphoribosylaminoimidazole carboxylase catalytic subunit, phosphoribosylformyl glycinamidine synthase subunit, phosphoribosylglycinamide formyltransferase, phosphoribosyltransferase, Photosystem I reaction center subunit XI, photosystem II protein, polygalacturonase, polyphosphate kinase, potassium transport protein, pre-mRNA-splicing factor, protease, protein kinase, protein phosphatase, purine nucleoside phosphorylase, purine-nucleoside phosphorylase, pyrroline carboxylate reductase, pyruvate kinase, quinolinate synthetase, riboflavin biosynthesis protein, ribosephosphate isomerase, constitutive, RNA binding protein, s_pp015018333r-protein, sec-independent protein translocase, Sec-independent protein translocase subunit, serine protease, serine protease inhibitor, short-chain alcohol dehydrogenase family, sll0064-protein, sll0254-protein, sll0354-protein, sll1761-protein, slr0600-protein, sodium/proton antiporter, squalene monooxygenase, sterol 0-acyltransferase, thioredoxin, thioredoxin family protein, threonine dehydrogenase, threonine synthase, transcription factor, transcriptional regulator, transcriptional regulator protein, translation initiation factor subunit, transport protein, TTC0768-protein, TTC1386-protein, urease subunit, uridine/cytidine kinase, valine-pyruvate transaminase, XM_{—}473199-protein, YCL026C-A-protein, ycr102c-protein, ydr338c-protein, yer014w-protein, yer106w-protein, YFL019C-protein, yfl054c-protein, ygl237c-protein, ygr068c-protein, ygr221 c-protein, yhl013c-protein, yhr207c-protein, ylr065c-protein, ylr178c-protein, ynl142w-protein, yor221c-protein, zinc finger protein, zinc transporter, Zm_{—}4842_BE510522-protein, and ZM06LC11975-protein are also called “FCRP genes”.

[0211.1.1.2] to [0225.1.1.2] for the disclosure of these paragraphs see [0211.1.1.1] to [0225.1.1.1] above.

In addition to the sequence mentioned in Table I, application no. 2, column 5 or 8, preferably the coding region thereof, or its homologs or fragments, it may be advantageous additionally to express and/or mutate further genes in the non-human organisms. Especially advantageously, additionally at least one further gene of the biosynthetic pathway of the fine chemical is expressed in the non-human organisms such as plants or microorganisms. It is also possible that the regulation of the natural genes has been modified advantageously so that the gene and/or its gene product is no longer subject to the regulatory mechanisms which exist in the non-human organisms. This leads to an increased synthesis of the respective amino acid since, for example, feedback regulations no longer exist to the same extent or not at all. In addition it might be advantageously to combine the nucleic acids sequences of the invention containing the sequences shown in the respective line in Table I, application no. 2, column 5 or 8, preferably the coding region thereof, or homologs or fragments thereof, with genes which generally support or enhance the growth or yield of the target non-human organisms, for example genes which lead to faster growth rate of non human organism like microorganisms or plants or genes which produce stress-, pathogen-, or herbicide-resistant plants.

[0227.1.1.2] to [0239.1.1.2] for the disclosure of these paragraphs see [0227.1.1.1] to [0239.1.1.1] above.

In an embodiment of the present invention the nucleic acid construct of the present invention comprises a nucleic acid molecule as depicted in the respective line in Table I, application no. 2, column 5 or 8, preferably the coding region thereof, a fragment or a homolog thereof, and a plant promoter, such as the Big 35S promoter, the DC3 promoter, the PC Ubi promoter, the LegB4-promoter, the Super promoter, the USP promoter or the like. In another embodiment the nucleic acid construct of the present invention comprises a nucleic acid molecule as depicted in th erespective line in Table I, application no. 2, column 5 or 8, preferably the coding region thereof, a fragment or a homolog thereof, and a plant promoter such as, for example, the USP, the LegB4-, the DC3 promoter or the ubiquitin promoter from parsley or other herein mentioned promoters. In a further embodiment the nucleic acid construct of the present invention comprises a nucleic acid molecule as depicted in th erespective line in Table I, application no. 2, column 5 or 8, preferably the coding region thereof, a fragment or a homolog thereof, and a plant promoter, such as the Big 35S promoter, the PC Ubi promoter, the Super promoter, the USP promoter or the like. In addition different terminators may advantageously be used in these nucleic acid constructs; examples for transcriptional termination are polyadenylation signals.

[0241.1.1.2] to [0245.1.1.2] for the disclosure of these paragraphs see [0241.1.1.1] to [0245.1.1.1] above.

Other preferred sequences for use in operable linkage in gene expression constructs are targeting sequences, which are required for targeting the gene product into specific cell compartments (for a review, see Kermode, Crit. Rev. Plant Sci. 15 (4), 285 (1996) and references cited therein), for example into the vacuole, the nucleus, all types of plastids, such as amyloplasts, chloroplasts, chromoplasts, the extracellular space, the mitochondria, the endoplasmic reticulum, elaioplasts, peroxisomes, glycosomes, and other compartments of cells or extracellular; preferred are sequences, which are involved in targeting to plastids as mentioned above. Sequences, which must be mentioned in this context are, in particular, the signal-peptide- or transit-peptide-encoding sequences which are known per se. For example, plastid-transit-peptide-encoding sequences enable the targeting of the expression product into the plastids of a plant cell. Targeting sequences are also known for eukaryotic and to a lower extent for prokaryotic organisms and can advantageously be operable linked with the nucleic acid molecule of the present invention as shown in Table I, application no. 2, columns 5 and 8, opreferably the coding region thereof, or homologs or fragments thereof, and described herein to achieve an expression in one of said compartments or extracellular.

[0247.1.1.2] to [0266.1.1.2] for the disclosure of these paragraphs see [0247.1.1.1] to [0266.1.1.1] above.

An embodiment of the invention relates to a vector where the nucleic acid molecule according to the invention is linked operably to regulatory sequences which permit the expression in a prokaryotic or eukaryotic host. A further embodiment of the invention relates to a vector in which a nucleic acid sequence encoding one of the polypeptides shown in therespective line in Table II, application no. 2, columns 5 or 8, or homologs or fragments thereof, is functionally linked to a targeting sequence such as a plastidial or mitochondrial targeting sequence. A further embodiment of the invention relates to a vector in which a nucleic acid sequence encoding one of the polypeptides shown in the respective line in Table II, application no. 2, columns 5 or 8, or homologs or fragments thereof, is functionally linked to (a) regulatory sequence which permit the expression in an organelle,such as plastids or mitochondria, preferably plastids.

A further embodiment of the invention relates to a vector which comprises a nucleic acid sequence encoding one of the polypeptides shown in the respective line in Table II, application no. 2, columns 5 or 8, or homologs or fragments thereof, but no additional targeting sequence.

[0268.1.1.2] to [0273.1.1.2] for the disclosure of these paragraphs see [0268.1.1.1] to [0273.1.1.1] above.

A further object of the invention relates to the use of a nucleic acid construct, e.g. an expression cassette, comprising one or more nucleic acid molecules encoding one or more polypeptides as depicted in the respective line(s) in Table II, application no. 2, columns 5 or 8, or homologs or fragments thereof, or comprising one or more nucleic acid molecules as depicted in the respective line(s) in Table I, application no. 2, columns 5 or 8, preferably the coding region thereof, or homologs or fragments thereof, or comprising a nucleic acid molecule hybridizing therewith for the transformation of a microorganism or a plant cell, plant tissues or a part of a plant

In doing so, depending on the choice of promoter, the nucleic acid molecules shown in the respective line in Table I, application no. 2, column 5 or 8, preferably the coding region thereof, or homologs or fragments thereof, or the nucleic acid molecules encoding a polypeptide as depicted in the respective line in Table II, application no. 2, column 5 or 8, or homologs or fragments thereof, can be expressed in the whole plant or specifically e.g. in the leaves, in the seeds, the nodules, in roots, in the stem or other parts of the plant. Those transgenic plants overproducing sequences, e.g. as depicted in the respective line in Table I, application no. 2, column 5 or 8, preferably the coding region thereof, or homologs or fragments thereof, the plant cell, plant tissuesplants, the reproductive material thereof, or parrts thereof are a further object of the present invention.

Within the framework of the present invention, the production of the arginine, glutamate, glutamine, or proline is due to the generation or over-expression of one or more polypeptides as depicted in the respective line(s) in Table II, application no. 2, column 5 or 8, or homologs or fragments thereof, or encoded by the corresponding nucleic acid molecules as depicted in the respective line(s) in Table I, application no. 2, column 5 or 8, preferably the coding region thereof, or homologs or fragments thereof, in the non-human organism according to the invention, advantageously in the transgenic microorganism or plant according to the invention, at least for the duration of at least one plant generation.

A constitutive expression of the polypeptide as depicted in the respective line in Table II, application no. 2, column 5 or 8, or homologs or fragments thereof, or encoded by the respective nucleic acid molecule as depicted in the respective line in Table I, application no. 2, column 5 or 8, preferably the coding region thereof, or homologs or fragments thereof, may be advantageous. On the other hand, an inducible expression may also appear desirable. Expression of the polypeptide sequences of the invention can be either directed to the cytosol or to the organelles, such as plastids or mitochondria, preferably the plastids of the host cells, preferably the plant cells, or non-targeted.

for the disclosure of this paragraph see [0278.1.1.1] above.

In a further embodiment the invention relates to a transgenic non-human organisms such as transgenic microorganism or transgenic plant comprising a nucleic acid molecule comprising

[1) a nucleic acid molecule selected from the group consisting of
(a) a nucleic acid molecule encoding a polypeptide shown in Table II, application no. 2, column 5 or 8, preferably shown in Table II A, application no. 2, column 5, or in Table II A, application no. 2, column 8, or in Table II B, application no. 2, column 8, or a homolog or a fragment thereof;
(b) a nucleic acid molecule shown in Table I, application no. 2, column 5 or 8, preferably shown in Table I A, application no. 2, column 5, or in Table I A, application no. 2, column 8, or in Table I B, application no. 2, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 2, preferably shown in Table II A, application no. 2, column 5, or in Table II A, application no. 2, column 8, or in Table II B, application no. 2, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 2, preferably shown in Table I A, application no. 2, column 5, or in Table I A, application no. 2, column 8,or in Table I B, application no. 2, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 2;
(i) a nucleic acid molecule encoding a polypeptide having the activity represented by a respective protein as depicted in column 5 of Table II, application no. 2;
(j) a nucleic acid molecule comprising a polynucleotide which is obtained by amplifying a cDNA library or a genomic library using the primers in Table III, application no. 2, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto; and, if desired
   2) nucleic acid molecule encoding a transit peptide, preferably encoding a plastidal transit peptide or a mitochondrial transit peptide; which are operable linked;]
   or comprising an expression cassette comprising
   [0) a promoter, prefereably a promoter selected from the group consisting of Big35S, PCUbi, Super and USP; and
   1) a nucleic acid molecule selected from the group consisting of
(a) a nucleic acid molecule encoding a polypeptide shown in Table II, application no. 2, column 5 or 8, preferably shown in Table II A, application no. 2, column 5, or in Table II A, application no. 2, column 8, or in Table II B, application no. 2, column 8, or a homolog or a fragment thereof;
(b) a nucleic acid molecule shown in Table I, application no. 2, column 5 or 8, preferably shown in Table I A, application no. 2, column 5, or in Table I A, application no. 2, column 8, or in Table I B, application no. 2, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 2, preferably shown in Table II A, application no. 2, column 5, or in Table II A, application no. 2, column 8, or in Table II B, application no. 2, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 2, preferably shown in Table I A, application no. 2, column 5, or in Table I A, application no. 2, column 8,or in Table I B, application no. 2, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 2;
(i) a nucleic acid molecule encoding a polypeptide having the activity represented by a respective protein as depicted in column 5 of Table II, application no. 2;
(j) a nucleic acid molecule comprising a polynucleotide which is obtained by amplifying a cDNA library or a genomic library using the primers in Table III, application no. 2, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto; and, if desired 2) nucleic acid molecule encoding a transit peptide, preferably encoding a plastidal transit peptide or a mitochondrial transit peptide;
   which are operable linked;]
   or comprising or being transformed by a vector comprising
   [1) a nucleic acid molecule selected from the group consisting of
(a) a nucleic acid molecule encoding a polypeptide shown in Table II, application no. 2, column 5 or 8, preferably shown in Table II A, application no. 2, column 5, or in Table II A, application no. 2, column 8, or in Table II B, application no. 2, column 8, or a homolog or a fragment thereof;
(b) a nucleic acid molecule shown in Table I, application no. 2, column 5 or 8, preferably shown in Table I A, application no. 2, column 5, or in Table I A, application no. 2, column 8, or in Table I B, application no. 2, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 2, preferably shown in Table II A, application no. 2, column 5, or in Table II A, application no. 2, column 8, or in Table II B, application no. 2, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 2, preferably shown in Table I A, application no. 2, column 5, or in Table I A, application no. 2, column 8, or in Table I B, application no. 2, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 2;
(i) a nucleic acid molecule encoding a polypeptide having the activity represented by a respective protein as depicted in column 5 of Table II, application no. 2;
(j) a nucleic acid molecule comprising a polynucleotide which is obtained by amplifying a cDNA library or a genomic library using the primers in Table III, application no. 2, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
   as well as respective transgenic cells, tissue, parts of such non-human organism, e.g. plant cells, plant tissue, part of plants, like leaves, roots, stems, blossoms, seeds, fruits, pollen and the like.
   (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 2.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 2 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 2, in the same line as of the respective nucleic acid molecule according to 1) “plastidic” is depicted, or encoding a mitochondrial transit peptide, especially in case in column 6 of Table I, application no. 2, in the same line as of the respective nucleic acid molecule according to 1) “mitochondrial” is depicted.

In another embodiment of said transgenic non-human organism the expression cassette does not comprise 2) a nucleic acid molecule encoding a transit peptide, especially in case in column 6 of Table I, application no. 2, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 2.

In another preferred embodiment of said transgenic non-human organism said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 2, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 2, in the same line as of the respective nucleic acid molecule according to 1) “plastidic” is depicted, or encoding a mitochondrial transit peptide, especially in case in column 6 of Table I, application no. 2, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 2, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 2, in the same line as of the respective nucleic acid molecule according to 1) “plastidic” is depicted, or encoding a mitochondrial transit peptide, especially in case in column 6 of Table I, application no. 2, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, especially in case in column 6 of Table I, application no. 2, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 2, in the same line as of the respective nucleic acid molecule according to 1) “plastidic” is depicted, or encoding a mitochondrial transit peptide, especially in case in column 6 of Table I, application no. 2, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 2.

In another embodiment of said transgenic non-human organism said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, especially in case in column 6 of Table I, application no. 2, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 2.

In another embodiment of said transgenic non-human organism said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 2, in the same line as of the respective nucleic acid molecule according to 1) “plastidic” is depicted, or encoding a mitochondrial transit peptide, especially in case in column 6 of Table I, application no. 2, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, especially in case in column 6 of Table I, application no. 2, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 2, in the same line as of the respective nucleic acid molecule according to 1) “plastidic” is depicted, or encoding a mitochondrial transit peptide, especially in case in column 6 of Table I, application no. 2, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 2 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, especially in case in column 6 of Table I, application no. 2, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 2 (in the same line) and confers a generation or an increase of the respective fine chemical in a non-human organism or a part thereof as compared to a corresponding wild-type non-human organism or part thereof.

In a prefered embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression casssette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

In a further embodiment the invention relates to a transgenic non-human organisms such as transgenic microorganism or transgenic plant comprising a nucleic acid molecule comprising

[1) a nucleic acid molecule selected from the group consisting of
(a) a nucleic acid molecule encoding a polypeptide shown in Table II, application no. 2, column 5 or 8, preferably shown in Table II A, application no. 2, column 5, or in Table II A, application no. 2, column 8, or in Table II B, application no. 2, column 8, or a homolog or a fragment thereof;
(b) a nucleic acid molecule shown in Table I, application no. 2, column 5 or 8, preferably shown in Table I A, application no. 2, column 5, or in Table I A, application no. 2, column 8, or in Table I B, application no. 2, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 2, preferably shown in Table II A, application no. 2, column 5, or in Table II A, application no. 2, column 8, or in Table II B, application no. 2, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 2, preferably shown in Table I A, application no. 2, column 5, or in Table I A, application no. 2, column 8,or in Table I B, application no. 2, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 2;
(i) a nucleic acid molecule encoding a polypeptide having the activity represented by a respective protein as depicted in column 5 of Table II, application no. 2;
(j) a nucleic acid molecule comprising a polynucleotide which is obtained by amplifying a cDNA library or a genomic library using the primers in Table III, application no. 2, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto; and, if desired
   2) nucleic acid molecule encoding a transit peptide, preferably encoding a plastidal transit peptide or a mitochondrial transit peptide; which are operable linked;]
   or comprising an expression cassette comprising
   [0) a promoter, prefereably a promoter selected from the group consisting of Big35S, PCUbi, Super and USP; and
   1) a nucleic acid molecule selected from the group consisting of
(a) a nucleic acid molecule encoding a polypeptide shown in Table II, application no. 2, column 5 or 8, preferably shown in Table II A, application no. 2, column 5, or in Table II A, application no. 2, column 8, or in Table II B, application no. 2, column 8, or a homolog or a fragment thereof;
(b) a nucleic acid molecule shown in Table I, application no. 2, column 5 or 8, preferably shown in Table I A, application no. 2, column 5, or in Table I A, application no. 2, column 8, or in Table I B, application no. 2, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 2, preferably shown in Table II A, application no. 2, column 5, or in Table II A, application no. 2, column 8, or in Table II B, application no. 2, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 2, preferably shown in Table I A, application no. 2, column 5, or in Table I A, application no. 2, column 8,or in Table I B, application no. 2, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 2;
(i) a nucleic acid molecule encoding a polypeptide having the activity represented by a respective protein as depicted in column 5 of Table II, application no. 2;
(j) a nucleic acid molecule comprising a polynucleotide which is obtained by amplifying a cDNA library or a genomic library using the primers in Table III, application no. 2, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]and, if desired 2) nucleic acid molecule encoding a transit peptide, preferably encoding a plastidal transit peptide or a mitochondrial transit peptide; which are operable linked;]
   or comprising or being transformed by a vector comprising
   [1) a nucleic acid molecule selected from the group consisting of
(a) a nucleic acid molecule encoding a polypeptide shown in Table II, application no. 2, column 5 or 8, preferably shown in Table II A, application no. 2, column 5, or in Table II A, application no. 2, column 8, or in Table II B, application no. 2, column 8, or a homolog or a fragment thereof;
(b) a nucleic acid molecule shown in Table I, application no. 2, column 5 or 8, preferably shown in Table I A, application no. 2, column 5, or in Table I A, application no. 2, column 8, or in Table I B, application no. 2, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 2, preferably shown in Table II A, application no. 2, column 5, or in Table II A, application no. 2, column 8, or in Table II B, application no. 2, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 2, preferably shown in Table I A, application no. 2, column 5, or in Table I A, application no. 2, column 8,or in Table I B, application no. 2, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 2;
(i) a nucleic acid molecule encoding a polypeptide having the activity represented by a respective protein as depicted in column 5 of Table II, application no. 2;
(j) a nucleic acid molecule comprising a polynucleotide which is obtained by amplifying a cDNA library or a genomic library using the primers in Table III, application no. 2, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
   whereby, preferably, the nucleic acid molecule according to 1 (a), (b), (c), (d), (e), (f), (g), (h), (i), (j) or (k) distinguishes over the sequence depicted in Table I A and/or I B, application no. 2, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 2, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 2, column 5 or 8, or the coding region thereof, respectively. In a further embodiment the nucleic acid molecule does not encode the polypeptide sequence shown in Table II A and/or II B, application no. 2, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 2, column 5 or 8 and does not encode a protein of the sequence shown in Table II A and/or II B, application no. 2, column 5 or 8. Accordingly, in one embodiment, the protein encoded by a sequence of a nucleic acid according to 1 (a), (b), (c), (d), (e), (f), (g), (h), (i), (j) or (k) does not consist of the sequence shown in Table I A and/or I B, application no. 2, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 2, column 5 or 8 but less than 100%, preferably less than 99.999%, 99.99%, 99.9%, 99%, 98%, 97%, 96% or 95% identical to the sequence shown in Table II A and/or II B, application no. 2, column 5 or 8;
   as well as respective transgenic cells, tissue, parts of such non-human organism, e.g. plant cells, plant tissue, part of plants, like leaves, roots, stems, blossoms, seeds, fruits, pollen and the like.
   (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 2.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 2 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 2, in the same line as of the respective nucleic acid molecule according to 1) “plastidic” is depicted, or encoding a mitochondrial transit peptide, especially in case in column 6 of Table I, application no. 2, in the same line as of the respective nucleic acid molecule according to 1) “mitochondrial” is depicted.

In another embodiment thereof the expression cassette does not comprise 2) a nucleic acid molecule encoding a transit peptide, especially in case in column 6 of Table I, application no. 2, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 2.

In another preferred embodiment of said transgenic non-human organism said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, prefereably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the same line in column 5 of Table II, application no. 2, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 2, in the same line as of the respective nucleic acid molecule according to 1) “plastidic” is depicted, or encoding a mitochondrial transit peptide, especially in case in column 6 of Table I, application no. 2, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no.

2, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 2, in the same line as of the respective nucleic acid molecule according to 1) “plastidic” is depicted, or encoding a mitochondrial transit peptide, especially in case in column 6 of Table I, application no. 2, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, especially in case in column 6 of Table I, application no. 2, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 2, in the same line as of the respective nucleic acid molecule according to 1) “plastidic” is depicted, or encoding a mitochondrial transit peptide, especially in case in column 6 of Table I, application no. 2, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 2.

In another embodiment of said transgenic non-human organism said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, especially in case in column 6 of Table I, application no. 2, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 2.

In another embodiment of said transgenic non-human organism said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 2, in the same line as of the respective nucleic acid molecule according to 1) “plastidic” is depicted, or encoding a mitochondrial transit peptide, especially in case in column 6 of Table I, application no. 2, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, especially in case in column 6 of Table I, application no. 2, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, preferably encoding a plastidal transit peptide, especially in case in column 6 of Table I, application no. 2, in the same line as of the respective nucleic acid molecule according to 1) “plastidic” is depicted, or encoding a mitochondrial transit peptide, especially in case in column 6 of Table I, application no. 2, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 2 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, prefereably a promoter selected from the group consisting of Big35S, PCUbi, Super and USP; a nucleic acid molecule encoding a transit peptide, especially in case in column 6 of Table I, application no. 2, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 2 and confers a generation or an increase of the respective fine chemical in a non-human organism or a part thereof as compared to a corresponding wild-type non-human organism or part thereof.

In a prefered embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression casssette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

[0291.1.1.2] to [0299.1.1.2] for the disclosure of these paragraphs see [0291.1.1.1] to [0299.1.1.1] above.

The term “biological active portion” or “biological activity” means a polypeptide as depicted in the respective line in Table II, application no. 2, column 3 or a portion of said polypeptide which still has at least 10% or 20%, preferably 30%, 40%, 50% or 60%, especially preferably 70%, 75%, 80%, 90% or 95% of the enzymatic or biological activity of the natural or starting enzyme or protein.

[301.1.1.2] to [0304.1.1.2] for the disclosure of these paragraphs see [0301.1.1.1] to [0304.1.1.1] above.

Synthetic oligonucleotide primers for the amplification, e.g. as shown in the respective line in Table III, application no. 2, column 8, by means of polymerase chain reaction can be generated on the basis of a sequence shown herein, for example the sequence shown in the respective line in Table I, application no. 2, columns 5 or 8, or the sequences derived from Table II, application no. 2, columns 5 or 8, respectively.

Moreover, it is possible to identify a conserved region of a protein from various organism by carrying out protein sequence alignments with the polypeptide encoded by the nucleic acid molecules of the present invention, in particular with the sequences encoded by the nucleic acid molecule shown in the respective line in column 5 or 8 of Table I, application no. 2, from which conserved regions, and in turn, degenerate primers can be derived. Conserved regions are those, which show a very little variation in the amino acid in one particular position of several homologs from different origin. The consensus sequence and polypeptide motifs shown in the respective line in column 8 of Table IV, application no. 2, are derived from such alignments of homologous proteins.

In an embodiment of the present invention, in the method of the present invention the activity of a polypeptide comprising or consisting of a consensus sequence or at least one polypeptide motif shown in the respective line in Table IV, application no.2, column 8, is increased. In another embodiment thereof, the method of the present invention relates to a polypeptide comprising or consisting of a consensus sequence shown in the respective line in

Table IV, application no. 2, column 8 or comprising at least one polypeptide motif shown in the respective line in Table IV, application no. 2, column 8, whereby not more than 20, 15, 10, 9, 8, 7, 6, 5 4, 3, 2 or 1, or 0 of the amino acid positions indicated can be replaced by any amino acid. In another embodiment thereof, the method of the present invention relates to a polypeptide comprising or consisting of a consensus sequence shown in the respective line in Table IV, application no. 2, column 8 or comprising at least one polypeptide motif shown in the respective line in Table IV, application no. 2, column 8, whereby not more than 15%, 10%, 5%, 4%, 3%, 2% or 1%, or 0% of the amino acid positions defined as distinct specific amino acids are/is replaced by another amino acid. In another embodiment thereof, the method of the present invention relates to a polypeptide comprising or consisting of a consensus sequence shown in the respective line in Table IV, application no. 2, column 8 or comprising at least one polypeptide motif shown in the respective line in Table IV, application no. 2, column 8, whereby not more than than 20, 15, 10, 9, 8, 7, 6, 5, 4, 3, 2 or 1, or 0 amino acids are inserted into a consensus sequence or polypeptide motif.

In an embodiment of the present invention, in the method of the present invention the activity of a polypeptide comprising or consisting of a consensus sequence or at least one polypeptide motif shown in the respective line in Table IV, application no.2, column 8, is increased, whereby said polypeptide distinguishes over the sequence depicted in Table II, application no. 2, columns 5 or 8 by one or more amino acids.

In one embodiment, said polypeptide of the invention distinguishes over the sequence shown in the respective line in Table II, application no. 2, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II B, application no. 2, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in Table II, application no. 2, columns 5 or 8.

[0309.1.1.2] to [0321.1.1.2] for the disclosure of these paragraphs see [0309.1.1.1] to [0321.1.1.1] above.

Polypeptides having above-mentioned activity, i.e. conferring the production or the increased production of the fine chemical arginine, glutamate, glutamine, or proline as compared to a corresponding, e.g. non-transformed, wild type non-human organism, like a microorganism or a plant cell, plant or part thereof, can be encoded by other DNA sequences which hybridize to the sequences shown in the respective line in Table I, application no. 2, columns 5 and 8, preferably the coding region therof, at least under relaxed hybridization conditions and which encode the expression of polypeptides conferring the production or the increased production of the respective fine chemical arginine, glutamate, glutamine, or proline as compared to a corresponding, e.g. non-transformed, wild type non-human organism, like a microorganism or a plant cell, plant or part thereof.

[0323.1.1.2] to [0329.1.1.2] for the disclosure of these paragraphs see [0323.1.1.1] to [0329.1.1.1] above.

Further, the nucleic acid molecule of the invention comprises a nucleic acid molecule, which is a complement of one of the nucleotide sequences of above-mentioned nucleic acid molecules or a portion thereof. A nucleic acid molecule or its sequence which is complementary to one of the nucleotide molecules or sequences shown in the respective line in Table I, application no. 2, columns 5 or 8, preferably the coding rgion thereof, homologs or fragments thereof, is one which is sufficiently complementary to one of the nucleotide molecules or sequences shown in the respective line inTable I, application no. 2, columns 5 or 8, preferably the coding rgion thereof, homologs or fragments thereof, such that it can hybridize to one of the nucleotide sequences shown in Table I, application no. 2, columns 5 or 8, preferably the coding rgion thereof, homologs or fragments thereof, thereby forming a stable duplex. Preferably, the hybridization is performed under stringent hybrization conditions. However, a complement of one of the herein disclosed sequences is preferably a sequence complement thereto according to the base pairing of nucleic acid molecules well known to the skilled person. For example, the bases A and G undergo base pairing with the bases T and U or C, resp. and visa versa. Modifications of the bases can influence the base-pairing partner.

The nucleic acid molecule of the invention comprises a nucleotide sequence which is at least about 30%, 35%, 40%, 45%, 50%, 55%, 60%, 65%, 70%, 75%, 80%, 90%, 95%, 96%, 97%, 98%, 99%, 99.5% or more homologous to a nucleotide sequence shown in Table I, application no. 2, columns 5 or 8, preferably the coding region thereof, homologs or fragments thereof, and preferably has above-mentioned activity, in particular conferring the production or the increased production of the fine chemical arginine, glutamate, glutamine, or proline, respectively, after increasing the activity or an activity of a gene as shown in the respective line in Table I or of a gene product, e.g. as shown in the respective line in Table II, application no. 2, column 5 or 8, by for example in one embodiment expression either in the cytsol or in an organelle such as a plastid or mitochondria or both, preferably in a plastid, or in another embodiment non-targeted or targeted.

The nucleic acid molecule of the invention comprises a nucleotide sequence or molecule which hybridizes, preferably hybridizes under stringent conditions as defined herein, to one of the nucleic acid molecule shown in the respective line in Table I, preferably Table IB, application no. 2, columns 5 or 8, preferably the coding region therof, fragements or homolos therof, and encodes a protein having above-mentioned activity, e.g. conferring the production or the increased production of the respective fine chemical arginine, glutamate, glutamine, or proline as compared to a corresponding, e.g. non-transformed, wild type non-human organism, like a microorganism or a plant cell, plant or part thereof, by for example in one embodiment expression either in the cytsol or in an organelle such as a plastid or mitochondria or both, preferably in a plastid, or in another embodiment non-targeted or targeted, and optionally, the activity thereof is selected from the group consisting of 2,3-dihydroxy-2,3-dihydrophenylpropionatedehydrogenase, 2-oxoglutarate dehydrogenase E1 subunit, 30S ribosomal protein, 3-deoxy-7-phosphoheptulonate synthase, 3-phosphoglycerate dehydrogenase, 47266012-protein, 49747384_SOYBEAN-protein, 4-alpha-glucanotransferase, ABC transporter permease protein, acetyl CoA carboxylase, acetyltransferase, acid shock protein, aconitate hydratase, acyl-CoA dehydrogenase, acyl-CoA synthase, acyltransferase, adenylosuccinate lyase, adenylylsulfate kinase, alanine dehydrogenase, aldehyde dehydrogenase, allantoicase, allantoinase, amine oxidase, amino acid ABC transporter permease protein, amino acid acetyltransferase, aminotransferase, ankyrin repeat family protein, anthranilate synthase component II, arginine decarboxylase, arginine exporter protein, asparaginase, aspartase, aspartate aminotransferase, At1g17440-protein, At1g19800-protein, At1g29350-protein, At1g47380-protein, At1g67340-protein, At4g32480-protein, At5g16650-protein, ATP synthase subunit beta, ATP-binding component of a transport system, ATP-dependent RNA helicase, AX653549-protein, AY087308-protein, b0456-protein, b0518-protein, b1003-protein, b1024-protein, b1108-protein, b1137-protein, b1163-protein, b1259-protein, b1280-protein, b1330-protein, b1445-protein, b1522-protein, b1898-protein, b2107-protein, b2121-protein, b2360-protein, b2399-protein, b2474-protein, b2513-protein, b2548-protein, b2613-protein, b2673-protein, b2812-protein, b2846-protein, b2909-protein, b2936-protein, b2999-protein, b3121-protein, b3151-protein, b3346-protein, b3410-protein, B3427-protein, b3509-protein, b3814-protein, b3817-protein, b3989-protein, b4029-protein, b4121-protein, beta-galactosidase, beta-hydroxylase, bifunctional purine biosynthesis protein, branched-chain amino acid ABC transporter permease protein, branched-chain amino acid permease, calcium-dependent protein kinase, carbohydrate kinase, carbon dioxide concentrating mechanism protein, cation/acetate symporter, cation-transporting ATPase, CBL-interacting protein kinase, CCAAT-binding transcription factor, CDP-diacylglycerol-glycerol-3-phosphate 3-phosphatidyltransferase, CDP-diglyceride synthetase, cell division control protein, cell division protein, Chaperone protein CIpB, circadian clock protein, coproporphyrinogen III oxidase, coproporphyrinogen oxidase, CTP synthase, CTP synthetase, cullin, cyclin D, cysteine synthase A, delta-1-pyrroline 5-carboxylase synthetase, dihydrolipoamide acetyltransferase, dihydrolipoamide dehydrogenase, DNA binding protein, DNA helicase II, DNA helicase IV, DNA mismatch repair protein, DnaJ-like chaperone, electron transport complex protein, elongation factor Tu, enoyl-CoA hydratase, enterobacterial common antigen polymerase, ethanolamine utilization protein, eukaryotic translation initiation factor 5, exopolyphosphatase, fatty acid desaturase, flavodoxin, fumarylacetoacetate hydrolase, gamma-glutamyltranspeptidase, geranylgeranyl pyrophosphate synthase, gibberellin 20-oxidase, Glu/Leu/Phe/Val dehydrogenase, gluconate transport system permease 3, glucose dehydrogenase, glucose-1-phosphate cytidylyltransferase, glucose-6-phosphate 1-dehydrogenase, glutamate-ammonia-ligase, glutamine amidotransferase, glutamine synthetase, glutaminyl-tRNA synthetase, glutaredoxin, glutathione S-transferase, glycerol dehydrogenase, glycerol-3-phosphate dehydrogenase, glycogen (starch) synthase, glycogen synthase, glycogenin, glycoprotease, glycosidase, glycosyl transferase, GM02LC11114-protein, GM02LC17485-protein, GM02LC46-protein, GM02LC5744-protein, H/ACA ribonucleoprotein complex subunit 3, harpin-induced family protein, heat shock protein, heat shock transcription factor, HesB/YadR/YfhF family protein, histone H2A, homocitrate synthase, hydrolase, integral membrane transporter family protein, iron(III) dicitrate-binding protein, isochorismate synthase, isocitrate dehydrogenase, isopentenyl diphosphate isomerase, L-asparaginase, L-aspartate oxidase, Leucyl/phenylalanyl-tRNA-protein transferase, lipoprotein precursor, L-ribulose-5-phosphate 4-epimerase, lysyl-tRNA synthetase, major facilitator superfamily transporter protein, malate dehydrogenase, malic enzyme, malonyl CoA-acyl carrier protein transacylase, membrane protein, membrane transport protein, metal-dependent hydrolase, methylglyoxal synthase, methyltransferase, mitochondrial processing protease, molecular chaperone portein, monothiol glutaredoxin, monthiol glutaredoxin, multiple antibiotic resistance protein, murein transglycosylase, N-acetyl-gamma-glutamyl-phosphate reductase, NAD(P)H-quinone oxidoreductase subunit, NADH dehydrogenase I chain I, nitrate/nitrite transport protein, ornithine carbamoyltransferase, oxidoreductase, oxidoreductase subunit, oxireductase, permease protein of phosphate ABC transporter, peroxisome assembly protein, phosphatidylinositol 3- and 4-kinase family protein, phosphoadenosine phosphosulfate reductase, phosphoanhydride phosphorylase, phosphopantetheine adenylyltransferase, phosphopantothenoylcysteine decarboxylase, phosphopantothenoylcysteinesynthetase/decarboxylase, phosphoribosylaminoimidazole carboxylase catalytic subunit, phosphoribosylformyl glycinamidine synthase subunit, phosphoribosylglycinamide formyltransferase, phosphoribosyltransferase, Photosystem I reaction center subunit XI, photosystem II protein, polygalacturonase, polyphosphate kinase, potassium transport protein, prem-RNA-splicing factor, protease, protein kinase, protein phosphatase, purine nucleoside phosphorylase, purine-nucleoside phosphorylase, pyrroline carboxylate reductase, pyruvate kinase, quinolinate synthetase, riboflavin biosynthesis protein, ribosephosphate isomerase, constitutive, RNA binding protein, s_pp015018333r-protein, sec-independent protein translocase, Sec-independent protein translocase subunit, serine protease, serine protease inhibitor, short-chain alcohol dehydrogenase family, sll0064-protein, sll0254-protein, sll0354-protein, sll1761-protein, slr0600-protein, sodium/proton antiporter, squalene monooxygenase, sterol O-acyltransferase, thioredoxin, thioredoxin family protein, threonine dehydrogenase, threonine synthase, transcription factor, transcriptional regulator, transcriptional regulator protein, translation initiation factor subunit, transport protein, TTC0768-protein, TTC1386-protein, urease subunit, uridine/cytidine kinase, valine-pyruvate transaminase, XM_{—}473199-protein, YCL026C-A-protein, ycr102c-protein, ydr338c-protein, yer014w-protein, yer106w-protein, YFLO19C-protein, yf1054c-protein, ygl237c-protein, ygr068c-protein, ygr221 c-protein, yhl013c-protein, yhr207c-protein, ylr065c-protein, ylr178c-protein, ynl142w-protein, yor221c-protein, zinc finger protein, zinc transporter, Zm_{—}4842_BE510522-protein, and ZM06LC11975-protein, respectively.

Moreover, the nucleic acid molecule of the invention can comprise only a portion of the coding region of one of the nucleic acid molecules depicted in Table I, application no. 2, columns 5 or 8, for example a fragment which can be used as a probe or primer or a fragment encoding a biologically active portion of the polypeptide of the present invention or of a polypeptide used in the process of the present invention, i.e. having above-mentioned activity, e.g. conferring the production or the increased production of the respective fine chemical arginine, glutamate, glutamine, or proline as compared to a corresponding, e.g. non-transformed, wild type non-human organism, like a microorganism or a plant cell, plant or part thereof, by for example in one embodiment expression either in the cytsol or in an organelle such as a plastid or mitochondria or both, preferably in a plastid, or in another embodiment non-targeted or targeted. The nucleotide sequences determined from the cloning of the present protein-accordingt-to-the-invention-encoding gene allows for the generation of probes and primers designed for use in identifying and/or cloning its homologues from other cell types and organisms. The probe/primer typically comprises a substantially purified oligonucleotide. The oligonucleotide typically comprises a region of nucleotide sequence that hybridizes under stringent conditions to at least about 12, 15 preferably about 20 or 25, more preferably about 40, 50 or 75 consecutive nucleotides of a sense strand of one of the sequences set forth, e.g., in the respective line in Table I, application no. 2, column 5 or 8, preferably the coding region thereof, or a homolog or a fragment thereof, an anti-sense sequence of one of the sequences, e.g., set forth in the respective line in Table I, application no. 2, columns 5 or 8, preferably the coding region thereof, or a homolog or a fragment thereof or naturally occurring mutants thereof. Primers based on a nucleic acid molecule of the invention can be used in PCR reactions to clone homologs of the polypeptide of the invention or of the polypeptide used in the process of the invention, e.g. as the primers described in the examples of the present invention, e.g. as shown in the examples. A PCR with the primers shown in the respective line in Table Ill, column 8 will result in a fragment of the gene product as shown in Table II, application no. 2, column 5.

Primer sets are interchangeable. The person skilled in the art knows how to desing and combine said primers to result in the desired product, e.g. in a full length clone or a partial sequence. Probes based on the sequences of the nucleic acid molecule of the invention or used in the process of the present invention can be used to detect transcripts or genomic sequences encoding the same or homologous proteins. The probe can further comprise a label group attached thereto, e.g. the label group can be a radioisotope, a fluorescent compound, an enzyme, or an enzyme co-factor. Such probes can be used as a part of a genomic marker test kit for identifying cells which express a polypeptide of the invention or used in the process of the present invention, such as by measuring a level of an encoding nucleic acid molecule in a sample of cells, e.g. detecting mRNA levels or determining, whether a genomic gene comprising the sequence of the polynucleotide of the invention or used in the processes of the present invention has been mutated or deleted.

As the expression of a respective nucleic acids of the invention is related to the synthesis of the respective fine chemical arginine, glutamate, glutamine, or proline its function as a probe extends to the detection of microorganisms, plant tissues, plants, plant variets, plant ecotypes or plant genera with varying capability or potential for synthesis of the respective fine chemical arginine, glutamate, glutamine, or proline. Therefore in one embodiment the present invention relates to a method for analyzing the capability or potential of a plant tissue, a plant, a plant variety or plant ecotype to produce the fine chemical arginine, glutamate, glutamine, or proline by using the nucleic acid of the invention or parts thereof as a probe to detect the amount of the nucleic acid of the invention in the non-human organism or a part thereof in comparision to another non-human organism.

The nucleic acid molecule of the invention encodes a polypeptide or portion thereof which includes an amino acid sequence which is sufficiently homologous to the amino acid sequence shown in respective line in Table II, application no. 2, columns 5 or 8 such that the protein or portion thereof maintains the ability to confer the production or the increased production of the respective fine chemical arginine, glutamate, glutamine, or proline as compared to a corresponding, e.g. non-transformed, wild type non-human organism, like a microorganism or a plant cell, plant or part thereof, in particular increasing the activity as mentioned above or as described in the examples in microorganisms or plants.

As used herein, the language “sufficiently homologous” refers to proteins or portions thereof which have amino acid sequences which include a minimum number of identical or equivalent amino acids (e.g. an amino acid which has a similar side chain as an amino acid in one of the sequences of the polypeptide of the present invention) to an amino acid sequence shown in the respective line in Table II, application no. 2, columns 5 or 8 such that the protein or portion thereof is able to confer the production or the increased production of the respective fine chemical arginine, glutamate, glutamine, or proline as compared to a corresponding, e.g. non-transformed, wild type non-human organism, like a microorganism or a plant cell, plant or part thereof. For example having the activity of a protein as shown in the respective line in Table II, application no. 2, column 3 and as described herein.

In an embodiment of the present invention, the nucleic acid molecule of the present invention comprises a nucleic acid molecule that encodes a portion of the protein of the present invention. The protein is at least about 30%, 35%, 40%, 45% 50%, 55%, 60%, 65%, 70%, 75%, 80%, 85%, 90%, 91%, 92%, 93%, 94%, 95%, 96%, 97%, 98%, 99%, 99.5% or more homologous to an entire amino acid sequence shown in the respective line of Table II, application no. 2, column 5 or 8 and has the above-mentioned activity, e.g. conferring the production or the increased production of the respective fine chemical arginine, glutamate, glutamine, or proline as compared to a corresponding, e.g. non-transformed, wild type non-human organism, like a microorganism or a plant cell, plant or part thereof by, for example in a embodiment expression either in the cytsol or in an organelle such as a plastid or mitochondria or both, preferably in a plastid, or in another embodiment by targeted or non-targeted expression.

[0338.1.1.2] to [0339.1.1.2] for the disclosure of these paragraphs see [0338.1.1.1] to [0339.1.1.1] above.

The invention further relates to nucleic acid molecules or methods using said nucleic acid molecules that differ from one of the nucleotide sequences shown in the respective line in Table I, application no. 2, columns 5 or 8, preferably the coding region thereof, or fragments or homologs thereof, due to degeneracy of the genetic code but encode a polypeptide of the present invention, in particular a polypeptide having the above-mentioned activity, e.g. as those polypeptides depicted in the respective line in Table II, application no.2, columns 5 or 8, fragments or homologs thereof. Advantageously, the nucleic acid molecule of the invention comprises, or in an other embodiment has, a nucleotide sequence encoding a protein comprising, or in an other embodiment having, an amino acid sequence shown in the respective line in Table II, application no. 2, columns 5 or 8, fragments or homologs thereof. In a further embodiment, the nucleic acid molecule of the invention encodes a full length protein which is substantially homologous to an amino acid sequence shown in the respective line in Table II, application no. 2, columns 5 or 8, or homologs thereof. However, in an embodiment, the nucleic acid molecule of the present invention does not consist of the sequence shown in the respective line in Table I, preferably Table IA, application no. 2, columns 5 or 8, preferably the coding region thereof, but the functional homologs thereof.

[0341.1.1.2] to [0343.1.1.2] for the disclosure of these paragraphs see [0341.1.1.1] to [0343.1.1.1] above.

Accordingly, in another embodiment, a nucleic acid molecule of the invention is at least 15, 20, 25 or 30 nucleotides in length. Preferably, it hybridizes under stringent conditions to a nucleic acid molecule comprising a nucleotide sequence of the nucleic acid molecule of the present invention or used in the process of the present invention, e.g. comprising the sequence shown in the respective line in Table I, application no. 2, columns 5 or 8, preferably the coding region thereof. In another embodiment the nucleic acid molecule is preferably at least 20, 30, 50, 100, 250 or more nucleotides in length.

for the disclosure of this paragraph see [0345.1.1.1] above.

Preferably, a nucleic acid molecule of the invention that hybridizes under stringent conditions to a sequence shown in the respective line in Table I, application no. 2, columns 5 or 8, preferably the coding region thereof, corresponds to a naturally-occurring nucleic acid molecule of the invention. As used herein, a “naturally-occurring” nucleic acid molecule refers to a RNA or DNA molecule having a nucleotide sequence that occurs in nature (e.g. encodes a natural protein). Preferably, the nucleic acid molecule encodes a natural protein having above-mentioned activity, e.g. conferring the production or the increased production of the respective fine chemical arginine, glutamate, glutamine, or proline as compared to a corresponding, e.g. non-transformed, wild type non-human organism, like a microorganism or a plant cell, plant or part thereof after increasing the expression or activity thereof or the activity of a protein of the invention or used in the process of the invention, in an embodiment for example expression either in the cytsol or in an organelle such as a plastid or mitochondria, preferably in plastids, or, in another embodiment by targeted or non-targeted expression.

for the disclosure of this paragraph see [0347.1.1.1] above.

For example, nucleotide substitutions leading to amino acid substitutions at “non-essential” amino acid residues can be made in a sequence of the nucleic acid molecule of the invention or used in the process of the invention, e.g. shown in the respective line in Table I, application no. 2, columns 5 or 8, preferably the coding region thereof, or fragments or homologs thereof.

[0349.1.1.2] to [0350.1.1.2] for the disclosure of these paragraphs see [0349.1.1.1] to [0350.1.1.1] above.

Accordingly, the invention relates to nucleic acid molecules encoding a polypeptide having above-mentioned activity, in an non-human organisms or parts thereof by for example expression either in the cytsol or in an organelle such as a plastid or mitochondria or both, preferably in plastids, that contain changes in amino acid residues that are not essential for said activity. Such polypeptides differ in amino acid sequence from a sequence depicted in the respective line in Table II, especially Table IIA, application no. 2, columns 5 or 8, but retain said activity described herein. The nucleic acid molecule can comprise a nucleotide sequence encoding a polypeptide, wherein the polypeptide comprises an amino acid sequence at least about 50% identical to an amino acid sequence shown in the respective line in Table II, application no. 2, columns 5 or 8, and is capable of conferring the production or the increased production of the respective fine chemical arginine, glutamate, glutamine or proline as compared to a corresponding, e.g. non-transformed, wild type non-human organism, like a microorganism or a plant cell, plant or part thereof, after increasing its activity for example in an embodiment by expression either in the cytsol or in an organelle such as a plastid or mitochondria or both, preferably in plastids, or, in another embodiment by targeted or non-targeted expression. Preferably, the protein encoded by the nucleic acid molecule is at least about 60%, 70%, 80%, 90%, 95%, 96%, 97%, 98%, 99% or 99.5% identical to the sequence shown in the respective line in Table II, application no. 2, columns 5 or 8.

[0352.1.1.2] to [0357.1.1.2] for the disclosure of these paragraphs see [0352.1.1.1] to [0357.1.1.1] above.

Functional equivalents of the nucleic acid molecules according to the present invention and/or used in the process according to the present invention represent an embodiment of homologs. Functional equivalents derived from the nucleic acid molecules as shown in the respective line in Table I, application no. 2, columns 5 or 8, preferably the coding region thereof, according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the nucleic acid molecules as shown in the respective line in Table I, application no. 2, columns 5 or 8, preferably the coding region thereof, according to the invention and encode polypeptides having essentially the same properties as the polypeptide as shown in the respective line in Table II, application no. 2, columns 5 or 8.

Functional equivalents derived from one of the polypeptides as shown in the respective line in Table II, application no. 2, columns 5 or 8 according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the polypeptides as shown in the respective line in Table II, application no. 2, columns 5 or 8 according to the invention and having essentially the same properties as the polypeptide as shown in the respective line in Table II, application no. 2, columns 5 or 8.

for the disclosure of this paragraph see [0359.1.1.1] above.

A nucleic acid molecule encoding a homolog to a polypeptide depicted in the respective line in Table II, application no. 2, columns 5 or 8 can be created by introducing one or more nucleotide substitutions, additions or deletions into the nucleic acid molecule of the present invention, in particular of Table I, application no. 2, columns 5 or 8, in the respective line, such that one or more amino acid substitutions, additions or deletions are introduced into the encoded protein. Mutations can be introduced into the nucleic acid molecules as depicted in the respective line in Table I, application no. 2, columns 5 or 8, preferably the coding region thereof, by standard techniques, such as site-directed mutagenesis,PCR-mediated mutagenesis or other methods known to the person skilled in the art.

[0361.1.1.2] to [0363.1.1.2] for the disclosure of these paragraphs see [0361.1.1.1] to [0363.1.1.1] above.

Homologues of the nucleic acid sequences used, with the sequences shown in the respective line in Table I, application no. 2, columns 5 or 8, perferably the coding region thereof, comprise also allelic variants with at least approximately 30%, 35%, 40%, 45%, 50%, 55%, 60%, 65%, 70%, 75%, 80%, 85%, 90%, 91%, 92%, 93%, 94% 95%. 96%, 97%, 98%, 99%, 99.5% or more % homology with one of the nucleotide sequences shown or the above-mentioned derived nucleic acid sequences or their homologues or derivatives or parts of these. Allelic variants encompass in particular functional variants which can be obtained by deletion, insertion or substitution of nucleotides from the sequences shown in the respective line, preferably in Table I, columns 5 or 8, preferably the coding region theeof, or from the derived nucleic acid sequences, the intention being, however, that the enzymatic activity or the biological activity of the resulting proteins synthesized is advantageously retained or increased.

In one embodiment of the present invention, the nucleic acid molecule of the invention or used in the process of the invention comprises the sequences shown in the respective line in any of the Table I, application no. 2, columns 5 or 8, preferably the coding region thereof, fragments or homologs thereof. It is preferred that the nucleic acid molecule comprises as little as possible other nucleotides not shown in the respective line in any one of Table I, application no. 2, columns 5 or 8, preferably the coding region thereof, fragments or homologs thereof. In one embodiment, the nucleic acid molecule comprises less than 500, 400, 300, 200, 100, 90, 80, 70, 60, 50 or 40 further nucleotides. In a further embodiment, the nucleic acid molecule comprises less than 30, 20 or 10 further nucleotides. In one embodiment, the nucleic acid molecule used in the process of the invention is identical to the sequences shown in the respective line in Table I, application no. 2, columns 5 or 8, preferably the coding region thereof.

Also preferred is that the nucleic acid molecule used in the process of the invention encodes a polypeptide comprising the sequence shown in the respective line in Table II, application no. 2, columns 5 or 8, fragments or homologs thereof. In one embodiment, the nucleic acid molecule encodes less than 150, 130, 100, 80, 60, 50, 40 or 30 further amino acids. In a further embodiment, the encoded polypeptide comprises less than 20, 15, 10, 9, 8, 7, 6 or 5 further amino acids. In one embodiment used in the inventive process, the encoded polypeptide is identical to the sequences shown in the respective line in Table II, application no. 2, columns 5 or 8.

In one embodiment, the nucleic acid molecule of the invention or used in the process encodes a polypeptide comprising the sequence shown in the respective line in Table II, application no. 2, columns 5 or 8, or fragments or homologs thereof, and comprises less than 100 further nucleotides. In a further embodiment, said nucleic acid molecule comprises less than 30 further nucleotides. In one embodiment, the nucleic acid molecule used in the process is identical to a coding sequence of the sequences shown in the respective line in Table I, application no. 2, columns 5 or 8.

Polypeptides (=proteins), which still have the essential biological or enzymatic activity of the polypeptide of the present invention conferring the production or the increased production of the respective fine chemical arginine, glutamate, glutamine, or proline as compared to a corresponding, e.g. non-transformed, wild type non-human organism, like a microorganism or a plant cell, plant or part thereof, i.e. whose activity is essentially not reduced, are polypeptides with at least 10%, 20%, 30%, 40%, 50%, 60%, 70%, 80%, 90%, 95% or more of the wild type biological activity or enzymatic activity, advantageously, the activity is essentially not reduced in comparison with the activity of a polypeptide shown in the respective line in Table II, application no.2, columns 5 or 8 expressed under identical conditions.

Homologues of nucleic acid molecules shown in the respective line in Table I, application no. 2, columns 5 or 8, preferably the coding region thereof, or fragments thereof, or of the derived sequences shown in the respective line in Table II, application no. 2, columns 5 or 8, or fragments thereof, also mean truncated sequences, cDNA, single-stranded DNA or RNA of the coding and noncoding DNA sequence. Homologues of said sequences are also understood as meaning derivatives, which comprise noncoding regions such as, for example, UTRs, introns, terminators, enhancers or promoter variants. The promoters upstream of the nucleotide sequences stated can be modified by one or more nucleotide substitution(s), insertion(s) and/or deletion(s) without, however, interfering with the functionality or activity either of the promoters, the open reading frame (=ORF) or with the 3′-regulatory region such as terminators or other 3′-regulatory regions, which are far away from the ORF. It is furthermore possible that the activity of the promoters is increased by modification of their sequence, or that they are replaced completely by more active promoters, even promoters from heterologous organisms. Appropriate promoters are known to the person skilled in the art and are mentioned herein below.

[0370.1.1.2] to [0379.1.1.2] for the disclosure of these paragraphs see [0370.1.1.1] to [0379.1.1.1] above.

Moreover, a native polypeptide conferring the increase of the respective fine chemical arginine, glutamate, glutamine, or proline in a non-human organism or a part thereof can be isolated from cells (e.g., endothelial cells), for example using the antibody of the present invention as described below, in particular, an antibody against proteins having 2,3-dihydroxy-2,3-dihydrophenylpropionatedehydrogenase, 2-oxoglutarate dehydrogenase E1 subunit, 30S ribosomal protein, 3-deoxy-7-phosphoheptulonate synthase, 3-phosphoglycerate dehydrogenase, 47266012-protein, 49747384_SOYBEAN-protein, 4-alpha-glucanotransferase, ABC transporter permease protein, acetyl CoA carboxylase, acetyltransferase, acid shock protein, aconitate hydratase, acyl-CoA dehydrogenase, acyl-CoA synthase, acyltransferase, adenylosuccinate lyase, adenylylsulfate kinase, alanine dehydrogenase, aldehyde dehydrogenase, allantoicase, allantoinase, amine oxidase, amino acid ABC transporter permease protein, amino acid acetyltransferase, aminotransferase, ankyrin repeat family protein, anthranilate synthase component II, arginine decarboxylase, arginine exporter protein, asparaginase, aspartase, aspartate aminotransferase, At1g17440-protein, At1g19800-protein, At1g29350-protein, At1g47380-protein, At1g67340-protein, At4g32480-protein, At5g16650-protein, ATP synthase subunit beta, ATP-binding component of a transport system, ATP-dependent RNA helicase, AX653549-protein, AY087308-protein, b0456-protein, b0518-protein, b1003-protein, b1024-protein, b1108-protein, b1137-protein, b1163-protein, b1259-protein, b1280-protein, b1330-protein, b1445-protein, b1522-protein, b1898-protein, b2107-protein, b2121-protein, b2360-protein, b2399-protein, b2474-protein, b2513-protein, b2548-protein, b2613-protein, b2673-protein, b2812-protein, b2846-protein, b2909-protein, b2936-protein, b2999-protein, b3121-protein, b3151-protein, b3346-protein, b3410-protein, B3427-protein, b3509-protein, b3814-protein, b3817-protein, b3989-protein, b4029-protein, b4121-protein, beta-galactosidase, betahydroxylase, bifunctional purine biosynthesis protein, branched-chain amino acid ABC transporter permease protein, branched-chain amino acid permease, calcium-dependent protein kinase, carbohydrate kinase, carbon dioxide concentrating mechanism protein, cation/acetate symporter, cation-transporting ATPase, CBL-interacting protein kinase, CCAAT-binding transcription factor, CDP-diacylglycerol-glycerol-3-phosphate 3-phosphatidyltransferase, CDP-diglyceride synthetase, cell division control protein, cell division protein, Chaperone protein CIpB, circadian clock protein, coproporphyrinogen III oxidase, coproporphyrinogen oxidase, CTP synthase, CTP synthetase, cullin, cyclin D , cysteine synthase A, delta-1-pyrroline 5-carboxylase synthetase, dihydrolipoamide acetyltransferase, dihydrolipoamide dehydrogenase, DNA binding protein, DNA helicase II, DNA helicase IV, DNA mismatch repair protein, DnaJ-like chaperone, electron transport complex protein, elongation factor Tu, enoyl-CoA hydratase, enterobacterial common antigen polymerase, ethanolamine utilization protein, eukaryotic translation initiation factor 5, exopolyphosphatase, fatty acid desaturase, flavodoxin, fumarylacetoacetate hydrolase, gamma-glutamyltranspeptidase, geranylgeranyl pyrophosphate synthase, gibberellin 20-oxidase, Glu/Leu/Phe/Val dehydrogenase , gluconate transport system permease 3, glucose dehydrogenase, glucose-1-phosphate cytidylyltransferase, glucose-6-phosphate 1-dehydrogenase, glutamate-ammonia-ligase, glutamine amidotransferase, glutamine synthetase, glutaminyl-tRNA synthetase, glutaredoxin, glutathione S-transferase, glycerol dehydrogenase, glycerol-3-phosphate dehydrogenase, glycogen (starch) synthase, glycogen synthase, glycogenin, glycoprotease, glycosidase, glycosyl transferase, GM02LC11114-protein, GM02LC17485-protein, GM02LC46-protein, GM02LC5744-protein, H/ACA ribonucleoprotein complex subunit 3, harpin-induced family protein, heat shock protein, heat shock transcription factor, HesB/YadR/YfhF family protein, histone H2A, homocitrate synthase, hydrolase, integral membrane transporter family protein, iron(III) dicitrate-binding protein, isochorismate synthase, isocitrate dehydrogenase, isopentenyl diphosphate isomerase, L-asparaginase, L-aspartate oxidase, Leucyl/phenylalanyl-tRNA-protein transferase, lipoprotein precursor, L-ribulose-5-phosphate 4-epimerase, lysyl-tRNA synthetase, major facilitator superfamily transporter protein, malate dehydrogenase, malic enzyme, malonyl CoA-acyl carrier protein transacylase, membrane protein, membrane transport protein, metal-dependent hydrolase, methylglyoxal synthase, methyltransferase, mitochondrial processing protease, molecular chaperone portein, monothiol glutaredoxin, monthiol glutaredoxin, multiple antibiotic resistance protein, murein transglycosylase, N-acetyl-gamma-glutamyl-phosphate reductase, NAD(P)H-quinone oxidoreductase subunit, NADH dehydrogenase I chain I, nitrate/nitrite transport protein, ornithine carbamoyltransferase, oxidoreductase, oxidoreductase subunit, oxireductase, permease protein of phosphate ABC transporter, peroxisome assembly protein, phosphatidylinositol 3- and 4-kinase family protein, phosphoadenosine phosphosulfate reductase , phosphoanhydride phosphorylase, phosphopantetheine adenylyltransferase, phosphopantothenoylcysteine decarboxylase, phosphopantothenoylcysteinesynthetase/decarboxylase, phosphoribosylaminoimidazole carboxylase catalytic subunit, phosphoribosylformyl glycinamidine synthase subunit, phosphoribosylglycinamide formyltransferase, phosphoribosyltransferase, Photosystem I reaction center subunit XI, photosystem II protein, polygalacturonase, polyphosphate kinase, potassium transport protein, pre-mRNA-splicing factor, protease, protein kinase, protein phosphatase, purine nucleoside phosphorylase, purine-nucleoside phosphorylase, pyrroline carboxylate reductase, pyruvate kinase, quinolinate synthetase, riboflavin biosynthesis protein, ribosephosphate isomerase, constitutive, RNA binding protein, s_pp015018333r-protein, sec-independent protein translocase, Sec-independent protein translocase subunit, serine protease, serine protease inhibitor, short-chain alcohol dehydrogenase family, sll0064-protein, sll0254-protein, sll0354-protein, sll1761-protein, slr0600-protein, sodium/proton antiporter, squalene monooxygenase, sterol O-acyltransferase, thioredoxin, thioredoxin family protein, threonine dehydrogenase, threonine synthase, transcription factor, transcriptional regulator, transcriptional regulator protein, translation initiation factor subunit, transport protein, TTC0768-protein, TTC1386-protein, urease subunit, uridine/cytidine kinase, valine-pyruvate transaminase, XM_{—}473199-protein, YCL026C-A-protein, ycr102c-protein, ydr338c-protein, yer014w-protein, yer106w-protein, YFL019C-protein, yfl054c-protein, ygl237c-protein, ygr068c-protein, ygr221c-protein, yhl013c-protein, yhr207c-protein, ylr065c-protein, ylr178c-protein, ynl142w-protein, yor221c-protein, zinc finger protein, zinc transporter, Zm_{—}4842_BE510522-protein, or ZM06LC11975-protein activity, respectively, or an antibody against polypeptides as shown in the respective line in Table II, application no. 2, columns 5 or 8, or fragments or homologs thereof which can be produced by standard techniques utilizing the polypeptid of the present invention or fragment thereof, i.e., the polypeptide of this invention (FCRP). Preferred are monoclonal antibodies.

for the disclosure of this paragraph see [0381.1.1.1] above.

In one embodiment, the present invention relates to a polypeptide as depicted in the respective line in Table II, especially in Table IIA or especially in Table IIB, application no. 2, columns 5 or 8, or fragment or homolog thereof or as coded by the nucleic acid molecule shown in the respective line in Table I, especially Table IA or especially Table IB, application no. 2, columns 5 or 8, preferably the coding region thereof, or fragment or homolog thereof.

In an embodiment, the present invention relates to a polypeptide comprising or consisting of a consensus sequence shown in the respective line in Table IV, application no. 2, column 8 or at least one polypeptide motif shown in the respective line in Table IV, application no. 2, column 8.

In an embodiment thereof, the present invention relates to a polypeptide comprising or consisting of a consensus sequence shown in the respective line in Table IV, application no. 2, column 8 or comprising at least one polypeptide motif shown in the respective line in Table IV, application no. 2, column 8, whereby not more than 20, 15, 10, 9, 8, 7, 6, 5, 4, 3, 2, or 1, or 0 of the amino acids can be replaced by any amino acid.

In another embodiment thereof, the present invention relates to a polypeptide comprising or consisting of a consensus sequence shown in the respective line in Table IV, application no. 2, column 8 or comprising at least one polypeptide motif shown in the respective line in Table IV, application no. 2, column 8, whereby not more than 15%, 10%, 5%, 4%, 3%, 2% or 1% or 0% of the amino acids defined as distinct specific amino acidsare/is replaced by another amino acid.

In another embodiment thereof, the present invention relates to a polypeptide comprising or consisting of a consensus sequence shown in the respective line in Table IV, application no. 2, column 8 or comprising at least one polypeptide motif shown in the respective line in Table IV, application no. 2, column 8, whereby not more than 20, 15, 10, 9, 8, 7, 6, 5, 4, 3, 2, or 1, or 0 amino acids are inserted into the consensus sequence or polypeptide motif.

In an embodiment of the present invention, the polypeptide of the present invention comprises or consists of a consensus sequence or at least one polypeptide motif shown in the respective line in Table IV, application no. 2, column 8, whereby said polypeptide distinguishes over the sequence depicted in the respective line in Table II, application no. 2, columns 5 or 8 by one or more amino acids. In one embodiment, said polypeptide of the invention distinguishes over the sequence shown in the respective line in Table II, application no. 2, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 2, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in the respective line in Table II, application no. 2, columns 5 and 8.

In an embodiment, the invention relates to a polypeptide conferring an increase in the respective fine chemical arginine, glutamate, glutamine, or proline in a non-human organism, especially a microorganism or a plant, or a part thereof, being encoded by the nucleic acid molecule of the invention or used in the process of the invention and having a sequence which distinguishes over the sequence as shown in the respective line in Table II, application no. 2, columns 5 or 8 by one or more amino acids (but not exceeding 5%, preferably 4%, 3%, 2%, 1%, 0.5%, 0.2% or 0.1%). In an embodiment, said polypeptide of the invention does not comprise or consist of the sequence shown in the respective line in Table II, application no. 2, columns 5 or 8. In an embodiment, said polypeptide of the present invention is less than 100%, 99.999%, 99.99%, 99.9% or 99% identical. In one embodiment, said polypeptide which differs at least in one or more amino acids (but not exceeding 5%, preferably 4%, 3%, 2%, 1%, 0.5%, 0.2% or 0.1%) from the polypeptide shown in the respective line in Table II, application no. 2, columns 5 and 8 does not comprise a protein of the sequence shown in the respective line in Table II A and/or II B, application no. 2, columns 5 or 8.

In an embodiment, the present invention relates to a polypeptide having the activity of the protein as shown in the respective line in Table II, application no. 2, column 3, which distinguishes over the sequence depicted in the respective line in Table II, application no. 2, columns 5 or 8 by one or more amino acids, preferably by more than 5, 6, 7, 8 or 9 amino acids, preferably by more than 10, 15, 20, 25 or 30 amino acids, evenmore preferred are more than 40, 50, or 60 amino acids and, preferably, the sequence of the polypeptide of the invention distinguishes from the sequence shown in the respective line in Table II, application no. 2, columns 5 and 8 by not more than 80%, 70%, 60%, 50%, 40%, 30%, 20%, 10%, 5%, 4%, 3%, 2%, 1% or 0.5%.

In another embodiment the polypeptide of the invention takes the form of a preprotein consisting of a plastidial or mitochondrial transit peptide joint to a polypeptide having the activity of the protein as shown in Table II, column 3, from which the transit peptide is preferably cleaved off upon transport of the preprotein into the organelle for example into the plastid or mitochondrion.

In another embodiment the polypeptide of the invention takes not the form of a preprotein.

[0390.1.1.2] to [0391.1.1.2] for the disclosure of these paragraphs see [0390.1.1.1] to [0391.1.1.1] above.

A polypeptide of the invention can participate in the process of the present invention. The polypeptide or a portion thereof comprises preferably an amino acid sequence, which is sufficiently homologous to an amino acid sequence shown in the respective line in Table II A and/or II B, application no. 2, columns 5 or 8.

Further, the polypeptide can have an amino acid sequence which is encoded by a nucleotide sequence which hybridizes, preferably hybridizes under stringent conditions as described above, to a nucleotide sequence of the nucleic acid molecule of the present invention. Accordingly, the polypeptide has an amino acid sequence which is encoded by a nucleotide sequence that is at least about 35%, 40%, 45%, 50%, 55%, 60%, 65%, 70%, 75%, 80%, 85%, 90%, 91%, 92%, 93%, 94%, 95%, 96%, 97%, 98%, 99%, 99.5% or more homologous to one of the amino acid sequences as shown in the respective line in Table II A and/or II B, application no. 2, columns 5 or 8, or fragments thereof. The preferred polypeptide of the present invention preferably possesses at least one of the activities according to the invention and described herein. A preferred polypeptide of the present invention includes an amino acid sequence encoded by a nucleotide sequence which hybridizes, preferably hybridizes under stringent conditions, to a nucleotide sequence shown in the respective line in Table I A and/or I B, application no. 2, columns 5 or 8, preferably the coding region thereof, or fragments thereto or which is homologous thereto, as defined above.

The polypeptide of the present invention can vary from the sequences shown in the respective line in Table II A and/or II B, application no. 2, columns 5 or 8 in amino acid sequence due to natural variation or mutagenesis, as described in detail herein. Accordingly, the polypeptide comprise an amino acid sequence which is at least about 35%, 40%, 45%, 50%, 55%, 60%, 65%, 70%, 75%, 80%, 85% or 90, 91%, 92%, 93%, 94%, 95%, 96%, 97%, 98%, 99%, 99.5% or more homologous to an entire amino acid sequence shown in the respective line in Table II A and/or II B, application no. 2, columns 5 or 8.

Biologically active portions of a polypeptide of the present invention include peptides comprising amino acid sequences derived from the amino acid sequence of the polypeptide of the present invention or used in the process of the present invention, e.g., the amino acid sequence shown in the respective line in Table II, application no. 2, columns 5 or 8 or the amino acid sequence of a protein homologous thereto, which include fewer amino acids than a full length polypeptide of the present invention or used in the process of the present invention or the full length protein which is homologous to a polypeptide of the present invention or used in the process of the present invention depicted herein, and exhibit at least one activity of a polypeptide of the present invention or used in the process of the present invention.

for the disclosure of this paragraph see [0396.1.1.1] above.

Manipulation of the nucleic acid molecule of the invention may result in the production of a protein having differences from the protein as shown in the respective line in Table II, application no. 2, column 5 or 8. Differences shall mean at least one amino acid different from the sequences as shown in the respective line in Table II, application no. 2, column 5 or 8, preferably at least 2, 3, 4, 5, 6, 7, 8, 9 or 10 amino acids (especially but not exceeding 0.1%, preferably 0.2%, 0.5%, 1%, 2%, 3% 4% or 5%), more preferably at least 15, 20, 25, 30, 35, 40, 45 or 50 amino acids (especially but not exceeding 0.5%, preferably 1%, 2%, 3% 4%, 5%, 10% or 25%) different from the sequences as shown in the respective line in Table II, application no. 2, column 5 or 8. These proteins may be improved in efficiency or activity, or may be more stable and therefore present in greater numbers in the cell as compared to the wild-type cell.

Any mutagenesis strategies for the polypeptide of the present invention or the polypeptide used in the process of the present invention to result in increasing said activity are not meant to be limiting; variations on these strategies will be readily apparent to one skilled in the art. Using such strategies, and incorporating the mechanisms disclosed herein, the nucleic acid molecule and polypeptide of the invention may be utilized to generate algae, ciliates, fungi, other microorganisms like C. glutamicum or plants or parts thereof, expressing proteins as shown in the respective line in Table II, application no. 2, column 5 or 8, or mutated proteins thereof. The nucleic acid molecules and polypeptide molecules of the invention are expressed such that the yield, production, and/or efficiency of production of a desired compound is improved.

[0399.1.1.2] to [0401.1.1.2] for the disclosure of these paragraphs see [0399.1.1.1] to [0401.1.1.1] above.

In one embodiment, a protein (=polypeptide) as shown in the respective line in Table II, application no. 2, column 5 or 8 refers to a polypeptide having an amino acid sequence corresponding to the polypeptide of the invention, especially to a polypeptide as shown in Table II, application no. 2, colunms 5 or 8 or homologs thereof, or used in the process of the invention, whereas a “non- inventive protein or polypeptide” or “other polypeptide” refers to a polypeptide having an amino acid sequence corresponding to a protein which is not substantially homologous to a polypeptide of the invention, preferably which is not substantially homologous to a polypeptide or protein as shown in the respective line in Table II, application no. 2, column 5 or 8 or homologs thereof, e.g., a protein which does not confer the activity described herein and which is derived from the same or a different organism.

[0403.1.1.2] to [0409.1.1.2] for the disclosure of these paragraphs see [0403.1.1.1] to [0409.1.1.1] above.

As the nucleic acid molecules of the invention and/or to expression thereof is related to the synthesis of the respective fine chemical arginine, glutamate, glutamine, or proline its function as a probe extends to the detection of microorganisms, plant tissues, plants, plant variets, plant ecotypes or plant genera with varying, advantageously increased, capability or potential for synthesis of the respective fine chemical arginine, glutamate, glutamine, or proline.

Therefore in one embodiment the present invention relates to a method for analyzing the capability or potential of a plant tissue, a plant, a plant variety or ecotype to produce the respective fine chemical arginine, glutamate, glutamine, or proline by using the respective antibody of the invention as a probe to detect the amount of the polypeptide encoded by said nucleic acid molecule of the invention in a non-human organism in comparision to another organism.

Therefore in one embodiment the invention relates to a method to probe a non-human organism for its capability for the production of the respective fine chemical comprising the steps of
a) analyzing the sequence and/or the expression of the respective nucleic acid molecule of the invention in least two non-human organisms which differ in the capability to produce the respective fine chemical;
b) associate thedifferent sequence and/or expression level of the nucleic acid molecules of the invention with their different capability to produce the respective fine chemical;
c) analyse the sequence and/or the expression of the nucleic acid molecule of the invention in at least a third non-human organism;
d) predict the capability of the at least third organism to produce the respective fine chemical based by comparing the sequence and/or expression of the nucleic acid of the invention to the sequences and/or expression levels of the nucleic acid molecule of the invention of the at least two organisms analyzed in step a.

[0411.1.1.2] to [0430.1.1.2] for the disclosure of these paragraphs see [0411.1.1.1] to 0430.1.1.1] above.

Accordingly the present invention relates to any cell transgenic for any nucleic acid characterized as part of the invention, e.g. conferring the increase of the respective fine chemical arginine, glutamate, glutamine, or proline in a cell or a non-human organism or a part thereof, e.g. the nucleic acid molecule of the invention, the nucleic acid construct of the invention, the vector of the invention, the expression cassette according to the invention, or a nucleic acid molecule encoding the polypeptide of the invention, e.g. encoding a polypeptide having an activity as the protein as shown in the respective line in Table II, application no. 2, column 3.

Due to the above-mentioned activity the respective fine chemical arginine, glutamate, glutamine, or proline content in a cell or a non-human organism is increased. For example, due to modulation or manupulation, the cellular activity is increased, in a preferred embodiment in organelles such as plastids or mitochondria, e.g. due to an increased expression or specific activity or specific targeting of the subject matters of the invention in a cell or a non-human organism or a part thereof especially in organelles such as plastids or mitochondria, or in another embodiment in the cytosol. Transgenic for a polypeptide having a protein or a protein activity means herein that due to modulation or manipulation of the genome, the activity of protein as shown in the respective line in Table II, application no. 2, column 3 or a protein as shown in the respective line in Table II, application no. 2, column 3-like activity is increased in the cell or non-human organism or part thereof, especially in organelles such as plastids or mitochondria, or especuially in the cytosol. Examples are described above in context with the process of the invention.

for the disclosure of this paragraph see [0432.1.1.1] above.

A naturally occurring expression cassette—for example the naturally occurring combination of the promoter of the gene encoding a protein as shown in the respective line in Table II, application no. 2, column 5 or 8, preferably the coding region thereof, or fragments or homologs thereof with the corresponding encoding gene—becomes a transgenic expression cassette when it is modified by non-natural, synthetic “artificial” methods such as, for example, mutagenesis. Such methods have been described (U.S. Pat. No. 5,565,350; WO 00/15815; also see above).

[0434.1.1.2] to [0435.1.1.2] for the disclosure of these paragraphs see [0434.1.1.1] to [0435.1.1.1] above.

Transgenic plants comprising the respective fine chemical(s) synthesized in the process according to the invention can be marketed directly without isolation of the fine chemical synthesized. In the process according to the invention, plants are understood as meaning all plant parts, plant organs such as leaf, stalk, root, tubers or seeds or propagation material or harvested material or the intact plant. In this context, the seed encompasses all parts of the seed such as the seed coats, epidermal cells, seed cells, endosperm or embryonic tissue. The fine chemical produced in the process according to the invention may, however, also be isolated from the plant. In case of arginine, glutamate, glutamine, or proline this can be in free form or bound to proteins. Fine chemical(s) produced by this process can be harvested by harvesting the non-human organisms either from the culture in which they grow or from the field. For example, this can be done via squeezing, grinding and/or extraction, salt precipitation and/or ion-exchange chromatography of the plant parts, preferably the plant seeds, plant fruits, plant tubers and the like.

[0437.1.1.2] to [0440.1.1.2] for the disclosure of these paragraphs see [0437.1.1.1] to [0440.1.1.1] above.

[0442.1.1.2] to [0454.1.1.2] for the disclosure of these paragraphs see [0442.1.1.1] to to [0454.1.1.1] above.

In one embodiment, the present invention relates to a method for the identification of a gene product conferring the production of or an increase in the fine chemical production in a non-human cell, comprising the following steps:
(a) contacting, e.g. hybridising, a, some or all nucleic acid molecules of a sample, e.g. cells, tissues, plants or microorganisms or a nucleic acid library, which can contain a candidate gene encoding a gene product conferring the production of or an increase in the fine chemical after expression, with the nucleic acid molecule of the present invention as shown in the respective line in column 5 or 8 of Table I A or B of application no. 2, preferably the coding region thereof, or a fragment or homolog thereof;
(b) identifying the nucleic acid molecules, which hybridize under relaxed stringent conditions with the nucleic acid molecule of the present invention in particular to the nucleic acid molecule sequence shown in the respective line in Table I, application no. 2, columns 5 or 8, preferably the coding region thereof, of a fragment or a homolog thereof, preferably in Table I B, application no. 2, columns 5 or 8, preferably the coding region thereof, of a fragment or a homolog thereof, and, optionally, isolating the full length cDNA clone or complete genomic clone;
(c) introducing the candidate nucleic acid molecules or a fragment thereof in host cells, preferably in a plant cell or a microorganism, appropriate for producing the fine chemical;
(d) expressing the identified nucleic acid molecules in the host cells for which the production of or the increased production of the fine chemical is desired;
(e) assaying the the fine chemical level in the non-human host cells; and
(f) identifying the nucleic acid molecule and/or its gene product which expression confers the production of or an increase in the the fine chemical level in the non-human host cell after expression compared to the wild type.

for the disclosure of this paragraph see [0456.1.1.1] above.

In another embodiment, the present invention relates to a method for the identification of a gene product conferring the production of or an increase in the fine chemical production in a cell, comprising the following steps:
(a) identifiying a nucleic acid molecule of a non-human organism, which is at least 20%, 25%, 30%, 35%, 40%, 50%, 60%, 70%, 80%, 90%, 95%, 96%, 97%, 98%, 99%, 99.5% or more homolog to the nucleic acid molecule encoding a polypeptide comprising the polypeptide molecule as shown in the respective line in column 5 or 8 of Table II, application no. 2, or comprising a consensus sequence or a polypeptide motif as shown in the respective line in column 8 of Table IV, application no. 2, or being encoded by a nucleic acid molecule comprising a polynucleotide as shown in the respective line in column 5 or 8 of Table I, application no. 2, preferably the coding region thereof, or a homologue thereof as described herein, for example via homology search in a data bank;
(b) introducing the candidate nucleic acid molecule in non-human host cells, preferably in a plant cell or a microorganism, appropriate for producing the fine chemical;
(c) expressing the identified nucleic acid molecule in the non-human host cells;
(d) assaying the fine chemcial level in the non-human host cells; and
(e) identifying the nucleic acid molecule and its gene product which expression confers the production of or an increase in the the fine chemical level in the non-human host cell after expression compared to the wild type.

for the disclosure of this paragraph see [0458.1.1.1] above.

Further, the nucleic acid molecules disclosed herein, in particular the nucleic acid molecules shown in the respective line in column 5 or 8 of Table I A or B of application no. 2, may be sufficiently homologous to the sequences of related species such that these nucleic acid molecules may serve as markers for the construction of a genomic map in related organism or for association mapping. Furthermore natural variation in the genomic regions corresponding to nucleic acids disclosed herein, in particular the nucleic acid molecule shown in the respective line in column 5 or 8 of Table I A or B, of application no. 2 or homologous thereof, may lead to variation in the activity of the proteins disclosed herein, in particular the proteins comprising polypeptides as shown in the respective lins in column 5 or 8 of Table II A or B, of application no. 2 or their homologous, or comprising the consensus sequence or the polypeptide motif as shown in the respective line in column 8 of Table IV, and in consequence in a natural variation of an increased fine chemical production.

In consequence natural variation eventually also exists in form of more active allelic variants leading already to a relative increase in the fine chemical. Different variants of the nucleic acid molecules disclosed herein, in particular the nucleic acid molecules comprising the nucleic acid molecules as shown in the respective line in column 5 or 8 of Table I A or B of application no. 2, preferably the coding region thereof, which corresponds to different levels of increase in fine chemical, e.g. different levels of increase in fine chemical, can be indentified and used for marker assisted breeding for an enhanced production of the fine chemical.

Natural variation may also exist in the regulatory regions, e.g. the promotors of the natural genes comprising the nucleic acid molecules as shown in the respective line column 5 or 8 of Table I A or B of application no. 2, preferably the coding region thereof, which can similarily correspond to different levels of increase in the fine chemical, e.g. different levels of increase in the fine chemical can be identified and used for marker assisted breeding for an enhanced production of the fine chemical.

Accordingly, the present invention relates to a method for breeding plants for the production of the fine chemical, comprising
(a) selecting a first plant variety with an increased production of the fine chemical, e.g. based on an increased expression of a nucleic acid molecule of the invention as disclosed herein, in particular of a nucleic acid molecule comprising a nucleic acid molecule as shown in the respective line in column 5 or 8 of Table I A or B of application no. 2, preferably the coding region thereof, or fragments or homologs thereof, or a polypeptide comprising a polypeptide as shown in the respective line in column 5 or 8 of Table II A or B of application no. 2, or fragments or homologs thereof, or comprising a consensus sequence or a polypeptide motif as shown in the respective line in column 8 of Table IV, or a homolog thereof, as described herein;
(b) associating the level of increased production of the fine chemical, with the expression level or the genomic structure of a gene encoding said polypeptide or said nucleic acid molecule;
(c) crossing the first plant variety with a second plant variety, which significantly differs in its level of fine chemical; and
(d) identifying, which of the offspring varieties has got increased production of the fine chemical by the expression level of said polypeptide or nucleic acid molecule or the genomic structure of the gene(s) encoding said polypeptide or nucleic acid molecule of the invention.

[0462.1.1.2] to [0482.1.1.2] for the disclosure of these paragraphs see [0462.1.1.1] to [0482.1.1.1] above.

A further embodiment of this invention is related to genes which increase or generate the production of the fine chemical arginine, glutamine or glutamate, respectively, or proline, respectively, in plant cells, plants or part thereof. Phenotypes thereto are associated with yield of plants (=yield related phenotypes). In accordance with the invention, therefore, the respective genes identified in Table I, wherein in column 7 arginine, glutamine or glutamate, respectively, or proline, respectively, are mentioned, especially the coding region thereof, or homologs or fragments thereof, may be employed to enhance any yield-related phenotype. Increased yield may be determined in field trials of transgenic plants and suitable control plants.

Alternatively, a transgene's ability to increase yield may be determined in a model plant. An increased yield phenotype may be determined in the field test or in a model plant by measuring any one or any combination of the following phenotypes, in comparison to a control plant: yield of dry harvestable parts of the plant, yield of dry aerial harvestable parts of the plant, yield of underground dry harvestable parts of the plant, yield of fresh weight harvestable parts of the plant, yield of aerial fresh weight harvestable parts of the plant yield of underground fresh weight harvestable parts of the plant, yield of the plant's fruit (both fresh and dried), grain dry weight, yield of seeds (both fresh and dry), and the like.

The most basic yield-related phenotype is increased yield associated with the presence of the gene or a homolog or a fragment thereof as a transgene in the plant, i.e., the intrinsic yield of the plant. Intrinsic yield capacity of a plant can be, for example, manifested in a field test or in a model system by demonstrating an improvement of seed yield (e.g. in terms of increased seed/grain size, increased ear number, increased seed number per ear, improvement of seed filling, improvement of seed composition, embryo and/or endosperm improvements, and the like);

modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under non-stressed conditions), improvement in plant architecture.

Increased yield-related phenotypes may also be measured to determine tolerance to abiotic environmental stress. Abiotic stresses include drought, low temperature, nutrient deficiency, salinity, osmotic stress, shade, high plant density, mechanical stresses, and oxidative stress, and yield-related phenotypes are encompassed by tolerance to such abiotic stresses. Additional phenotypes that can be monitored to determine enhanced tolerance to abiotic environmental stress include, without limitation, wilting; leaf browning; loss of turgor, which results in drooping of leaves or needles stems, and flowers; drooping and/or shedding of leaves or needles; the leaves are green but leaf angled slightly toward the ground compared with controls; leaf blades begun to fold (curl) inward; premature senescence of leaves or needles; loss of chlorophyll in leaves or needles and/or yellowing. Any of the yield-related phenotypes described above may be monitored in field tests or in model plants to demonstrate that a transgenic plant has increased tolerance to abiotic environmental stress. In accordance with the invention, the respective genes identified in Table 1, especially the coding region thereof, or homologs or fragments thereof, wherein in the respective same line arginine, glutamine or glutamate,respectively or proline, respectively, are mentioned, may be employed to enhance tolerance to abiotic environmental stress in a plant when confronted with abiotic environmental stress.

A polypeptide conferring an yield-increasing activity can be encoded by a respective nucleic acid sequence as shown in Table I, column 5 or 8, and/or comprises or consists of a respective polypeptide as depicted in Table II, column 5 and 8, and/or can be amplified with the respective primer set shown in Table III, column 8, in case in column 7 the metabolite “glutamate”, “glutamine” or “arginine”, respectively or “proline”, respectively, is indicated.

“Improved adaptation” to environmental stress like e.g. freezing and/or chilling temperatures refers to an improved plant performance under environmental stress conditions.

A modification, i.e. an increase, can be caused by endogenous or exogenous factors. For example, an increase in activity in an organism or a part thereof can be caused by adding a gene product or a precursor or an activator or an agonist to the media or nutrition or can be caused by introducing said subjects into a organism, transient or stable. Furthermore such an increase can be reached by the introduction of the respective inventive nucleic acid sequence or the encoded protein in the correct cell compartment for example into the nucleus or cytoplasmic respectively or into plastids either by transformation and/or targeting.

The term “yield” as used herein generally refers to a measurable produce from a plant, particularly a crop. Yield and yield increase (in comparison to a non-transformed starting or wild-type plant) can be measured in a number of ways, and it is understood that a skilled person will be able to apply the correct meaning in view of the particular embodiments, the particular crop concerned and the specific purpose or application concerned. The terms “improved yield” or “increased yield” can be used interchangeable.

As used herein, the term “improved yield” or the term “increased yield” means any improvement in the yield of any measured plant product, such as grain, fruit or fiber. In accordance with the invention, changes in different phenotypic traits may improve yield. For example, and without limitation, parameters such as floral organ development, root initiation, root biomass, seed number, seed weight, harvest index, tolerance to abiotic environmental stress, leaf formation, phototropism, apical dominance, and fruit development, are suitable measurements of improved yield. Any increase in yield is an improved yield in accordance with the invention. For example, the improvement in yield can comprise a 0.1%, 0.5%, 1%, 3%, 5%, 10%, 15%, 20%, 30%, 40%, 50%, 60%, 70%, 80%, 90% or greater increase in any measured parameter. For example, an increase in the bushel/acre yield of soybeans or corn derived from a crop comprising plants which are transgenic for the nucleotides and polypeptides of Table I, in case in column 7 the metabolite “glutamate”, “glutamine” or “arginine”, respectively or “proline”, respectively, is indicated, as compared with the bushel/acre yield from untreated soybeans or corn cultivated under the same conditions, is an improved yield in accordance with the invention. The increased or improved yield can be achieved in the absence or presence of stress conditions.

For example, enhanced or increased “yield” refers to one or more yield parameters selected from the group consisting of biomass yield, dry biomass yield, aerial dry biomass yield, underground dry biomass yield, fresh-weight biomass yield, aerial fresh-weight biomass yield, underground fresh-weight biomass yield; enhanced yield of harvestable parts, either dry or freshweight or both, either aerial or underground or both; enhanced yield of crop fruit, either dry or fresh-weight or both, either aerial or underground or both; and preferably enhanced yield of seeds, either dry or fresh-weight or both, either aerial or underground or both.

Crop yield is defined herein as the number of bushels of relevant agricultural product (such as grain, forage, or seed) harvested per acre. Crop yield is impacted by abiotic stresses, such as drought, heat, salinity, and cold stress, and by the size (biomass) of the plant. Traditional plant breeding strategies are relatively slow and have in general not been successful in conferring increased tolerance to abiotic stresses. Grain yield improvements by conventional breeding have nearly reached a plateau in maize.

Accordingly, the yield of a plant can depend on the specific plant/crop of interest as well as its intended application (such as food production, feed production, processed food production, biofuel, biogas or alcohol production, or the like) of interest in each particular case. Thus, in one embodiment, yield is calculated as harvest index (expressed as a ratio of the weight of the respective harvestable parts divided by the total biomass), harvestable parts weight per area (acre, square meter, or the like); and the like. The harvest index, i.e., the ratio of yield biomass to the total cumulative biomass at harvest, in maize has remained essentially unchanged during selective breeding for grain yield over the last hundred years. Accordingly, recent yield improvements that have occurred in maize are the result of the increased total biomass production per unit land area. This increased total biomass has been achieved by increasing planting density, which has led to adaptive phenotypic alterations, such as a reduction in leaf angle, which may reduce shading of lower leaves, and tassel size, which may increase harvest index. Harvest index is relatively stable under many environmental conditions, and so a robust correlation between plant size and grain yield is possible. Plant size and grain yield are intrinsically linked, because the majority of grain biomass is dependent on current or stored photosynthetic productivity by the leaves and stem of the plant. As with abiotic stress tolerance, measurements of plant size in early development, under standardized conditions in a growth chamber or greenhouse, are standard practices to measure potential yield advantages conferred by the presence of a transgene.

Acccordingly, the yield of a plant can be increased by improving one or more of the yield-related phenotypes.

Such yield-related phenotypes or traits of a plant the improvement of which results in increased yield comprise, without limitation, the increase of the intrinsic yield capacity of a plant and/or increased stress tolerance, e.g. improved nutrient use efficiency, improved drought tolerance, improved cold tolerance.

For example, yield refers to biomass yield, e.g. to dry weight biomass yield and/or fresh-weight biomass yield. Biomass yield refers to the aerial or underground parts of a plant, depending on the specific circumstances (test conditions, specific crop of interest, application of interest, and the like). In one embodiment, biomass yield refers to the aerial and underground parts. Biomass yield may be calculated as fresh-weight, dry weight or a moisture adjusted basis. Biomass yield may be calculated on a per plant basis or in relation to a specific area (e.g. biomass yield per acre/square meter/or the like).

In other embodiment, “yield” refers to seed yield which can be measured by one or more of the following parameters: number of seeds or number of filled seeds (per plant or per area (acre/square meter/or the like)); seed filling rate (ratio between number of filled seeds and total number of seeds); number of flowers per plant; seed biomass or total seeds weight (per plant or per area (acre/square meter/or the like); thousand kernel weight (TKW; extrapolated from the number of filled seeds counted and their total weight; an increase in TKW may be caused by an increased seed size, an increased seed weight, an increased embryo size, and/or an increased endosperm). Other parameters allowing to measure seed yield are also known in the art. Seed yield may be determined on a dry weight or on a fresh weight basis, or typically on a moisture adjusted basis, e.g. at 15.5 percent moisture.

For example, the term “increased yield” means that the a plant, exhibits an increased growth rate, under conditions of abiotic environmental stress, compared to the corresponding wild-type plant.

An increased growth rate may be reflected inter alia by or confers an increased biomass production of the whole plant, or an increased biomass production of the aerial parts of a plant, or by an increased biomass production of the underground parts of a plant, or by an increased biomass production of parts of a plant, like stems, leaves, blossoms, fruits, and/or seeds.

Increased yield includes higher fruit yields, higher seed yields, higher fresh matter production, and/or higher dry matter production.

The term “increased yield” means that the plant, exhibits an prolonged growth under conditions of abiotic environmental stress, as compared to the corresponding, e.g. non-transformed, wild type organism. A prolonged growth comprises survival and/or continued growth of the plant, at the moment when the non-transformed wild type organism shows visual symptoms of deficiency and/or death.

When the plant of the invention is a corn plant, increased yield for corn plants means, for example, increased seed yield, in particular for corn varieties used for feed or food. Increased seed yield of corn refers to an increased kernel size or weight, an increased kernel per ear, or increased ears per plant. Alternatively or in addition the cob yield may be increased, or the length or size of the cob is increased, or the kernel per cob ratio is improved.

When the plant of the invention is a soy plant, increased yield for soy plants means increased seed yield, in particular for soy varieties used for feed or food. Increased seed yield of soy refers for example to an increased kernel size or weight, an increased kernel per pod, or increased pods per plant.

When the plant of the invention is an oil seed rape (OSR) plant, increased yield for OSR plants means increased seed yield, in particular for OSR varieties used for feed or food. Increased seed yield of OSR refers to an increased seed size or weight, an increased seed number per silique, or increased siliques per plant.

When the plant of the invention is a cotton plant. Increased yield for cotton plants means increased lint yield. Increased lint yield of cotton refers in one embodiment to an increased length of lint.

Said increased yield can typically be achieved by enhancing or improving, one or more yieldrelated traits of the plant. Such yield-related traits of a plant comprise, without limitation, the increase of the intrinsic yield capacity of a plant and/or increased stress tolerance, in particular increased abiotic stress tolerance, like for example improved nutrient use efficiency, e.g. nitrogen use efficiency, and/or improved water use efficiency, and or improved cold tolerance.

Intrinsic yield capacity of a plant can be, for example, manifested by improving the specific (intrinsic) seed yield (e.g. in terms of increased seed/ grain size, increased ear number, increased seed number per ear, improvement of seed filling, improvement of seed composition, embryo and/or endosperm improvements, or the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under stressed or non-stressed conditions), improvement in plant architecture, cell cycle modifications, photosynthesis modifications, various signaling pathway modifications, modification of transcriptional regulation, modification of translational regulation, modification of enzyme activities, and the like); and/or the like.

The improvement or increase of stress tolerance of a plant can for example be manifested by improving or increasing a plant's tolerance against stress, particularly abiotic stress. In the present application, abiotic stress refers generally to abiotic environmental conditions a plant is typically confronted with, including, but not limited to, drought (tolerance to drought may be achieved as a result of improved water use efficiency), heat, low temperatures and cold conditions (such as freezing and chilling conditions), nutrient depletion, salinity, osmotic stress, shade, high plant density, mechanical stress, oxidative stress, and the like.

The increased plant yield can for example be mediated by increasing the “nutrient use efficiency of a plant”, e.g. by improving the use efficiency of nutrients including, but not limited to, phosphorus, potassium, and nitrogen. Further, higher yields may be obtained with current or standard levels of nitrogen use.

Generally, the term “increased tolerance to stress” can be defined as survival of plants, and/or higher yield production, under stress conditions as compared to a non-transformed wild type or starting plant: For example, the plant of the invention or produced according to the method of the invention is better adapted to the stress conditions. “Improved adaptation” to environmental stress like e.g. drought, heat, nutrient depletion, freezing and/or chilling temperatures refers herein to an improved plant performance resulting in an increased yield, particularly with regard to one or more of the yield related traits as defined in more detail above.

During its life-cycle, a plant is generally confronted with a diversity of environmental conditions. Any such conditions, which may, under certain circumstances, have an impact on plant yield, are herein referred to as “stress” condition. Environmental stresses may generally be divided into biotic and abiotic (environmental) stresses. Unfavorable nutrient conditions are also referred to as “environmental stress”. The present invention does also contemplate solutions for this kind of environmental stress, e.g. referring to increased nutrient use efficiency.

For the purposes of the description of the present invention, the terms “enhanced tolerance to abiotic stress”, “enhanced resistance to abiotic environmental stress”, “enhanced tolerance to environmental stress”, “improved adaptation to environmental stress” and other variations and expressions similar in its meaning are used interchangeably and refer, without limitation, to an improvement in tolerance to one or more abiotic environmental stress(es) as described herein and as compared to a corresponding origin or wild type plant or a part thereof.

The term abiotic stress tolerance(s) refers for example low temperature tolerance, drought tolerance or improved water use efficiency (WUE), heat tolerance, salt stress tolerance, improved nutrient efficiency, especially improved nitrogen use efficiency (NUE) and others. Studies of a plant's response to desiccation, osmotic shock, and temperature extremes are also employed to determine the plant's tolerance or resistance to abiotic stresses. Water use efficiency (WUE) is a parameter often correlated with drought tolerance. In selecting traits for improving crops, a decrease in water use, without a change in growth would have particular merit in an irrigated agricultural system where the water input costs were high. An increase in growth without a corresponding jump in water use would have applicability to all agricultural systems. In many agricultural systems where water supply is not limiting, an increase in growth, even if it came at the expense of an increase in water use also increases yield.

Drought stress means any environmental stress which leads to a lack of water in plants or reduction of water supply to plants, including a secondary stress by low temperature and/or salt, and/or a primary stress during drought or heat, e.g. desiccation etc.

### Specific Embodiments

Accordingly, this invention provides respective measures and methods to produce plants with increased yield, e.g. genes conferring an increased yield-related trait, for example enhanced tolerance to abiotic environmental stress, for example an increased drought tolerance and/or low temperature tolerance and/or an increased nutrient use efficiency, and/or intrinsic yield and/or another increased yield-related trait, upon expression or over-expression. Accordingly, the present invention provides genes derived from plants in case in column 7 of the respective Table “arginine”, glutamate” or “glutamine”, respectively, or proline”, respectively, is indicated; in particular, respective genes are described in column 5 as well as in column 8 of Tables I or II.

Accordingly, the present invention provides respective transgenic plants showing one or more improved yield-related traits as compared to the corresponding origin or the wild type plant and methods for producing such transgenic plants with increased yield in case in column 7 of the respective Table “arginine”, “glutamate”, or “glutamine”, respectively, or proline”, respectively, is indicated.

In one embodiment, one or more of said yield-increasing activities are increased by increasing the amount and/or the specific activity of one or more proteins listed in Table I, column 5 or 8 in a compartment of a cell indicated in Table I, column 6 in case in column 7 of the respective Table “arginine”, “glutamate”, or “glutamine”, respectively, or proline”, respectively is indicated.

Accordingly to present invention, the yield of the plant of the invention is increased by improving one or more of the yield-related traits as defined herein. Said increased yield in accordance with the present invention can typically be achieved by enhancing or improving, in comparison to an origin or wild-type plant, one or more yield-related traits of said plant. Such yield-related traits of a plant the improvement of which results in increased yield comprise, without limitation, the increase of the intrinsic yield capacity of a plant, and/or increased stress tolerance, especially inctreased abiotic stress tolerance.

In a particular embodyment the present the invention relates to plants with a increased nutrient efficiency, especially a nitrogen use efficiency in case a plant is transformed with a gene being depited in Table I, and wherein in the same line of said gene in column 7 “arginine”, “glutamate”, or “glutamine” is indicated.

In another embodiment the present the invention relates to plants with an incresed water use efficiency/drought tolerance and/or low temperature tolerance in case a plant is transformed with a gene being depited in Table I, and wherein in the same line of said gene in column 7 “proline” is indicated.

In a further embodiment the present invention is characterized by the features as disclosed in the following items:

Item 1. A process for the production of the respective fine chemical arginine, glutamate, glutamine or proline, which comprises
(a) increasing or generating one or more respective activities selected from the group consisting of 2,3-dihydroxy-2,3-dihydrophenylpropionatedehydrogenase, 2-oxoglutarate dehydrogenase E1 subunit, 30S ribosomal protein, 3-deoxy-7-phosphoheptulonate synthase, 3-phosphoglycerate dehydrogenase, 47266012protein, 49747384_SOYBEAN-protein, 4-alpha-glucanotransferase, ABC transporter permease protein, acetyl CoA carboxylase, acetyltransferase, acid shock protein, aconitate hydratase, acyl-CoA dehydrogenase, acyl-CoA synthase, acyltransferase, adenylosuccinate lyase, adenylylsulfate kinase, alanine dehydrogenase, aldehyde dehydrogenase, allantoicase, allantoinase, amine oxidase, amino acid ABC transporter permease protein, amino acid acetyltransferase, aminotransferase, ankyrin repeat family protein, anthranilate synthase component II, arginine decarboxylase, arginine exporter protein, asparaginase, aspartase, aspartate aminotransferase, At1g17440-protein, At1g19800-protein, At1g29350-protein, At1g47380-protein, At1g67340-protein, At4g32480-protein, At5g16650-protein, ATP synthase subunit beta, ATP-binding component of a transport system, ATP-dependent RNA helicase,

AX653549-protein, AY087308-protein, b0456-protein, b0518-protein, b1003-protein, b1024-protein, b1108-protein, b1137-protein, b1163-protein, b1259-protein, b1280-protein, b1330-protein, b1445-protein, b1522-protein, b1898-protein, b2107-protein, b2121-protein, b2360-protein, b2399-protein, b2474-protein, b2513-protein, b2548-protein, b2613-protein, b2673-protein, b2812-protein, b2846-protein, b2909-protein, b2936-protein, b2999-protein, b3121-protein, b3151-protein, b3346-protein, b3410protein, B3427-protein, b3509-protein, b3814-protein, b3817-protein, b3989-protein, b4029-protein, b4121-protein, beta-galactosidase, beta-hydroxylase, bifunctional purine biosynthesis protein, branched-chain amino acid ABC transporter permease protein, branched-chain amino acid permease, calcium-dependent protein kinase, carbohydrate kinase, carbon dioxide concentrating mechanism protein, cation/acetate symporter, cation-transporting ATPase, CBL-interacting protein kinase, CCAAT-binding transcription factor, CDP-diacylglycerol-glycerol-3-phosphate 3-phosphatidyltransferase, CDP-diglyceride synthetase, cell division control protein, cell division protein, Chaperone protein CIpB, circadian clock protein, coproporphyrinogen III oxidase, coproporphyrinogen oxidase, CTP synthase, CTP synthetase, cullin, cyclin D, cysteine synthase A, delta-1-pyrroline 5-carboxylase synthetase, dihydrolipoamide acetyltransferase, dihydrolipoamide dehydrogenase, DNA binding protein, DNA helicase II, DNA helicase IV, DNA mismatch repair protein, DnaJ-like chaperone, electron transport complex protein, elongation factor Tu, enoyl-CoA hydratase, enterobacterial common antigen polymerase, ethanolamine utilization protein, eukaryotic translation initiation factor 5, exopolyphosphatase, fatty acid desaturase, flavodoxin, fumarylacetoacetate hydrolase, gammaglutamyltranspeptidase, geranylgeranyl pyrophosphate synthase, gibberellin 20-oxidase, Glu/Leu/Phe/Val dehydrogenase, gluconate transport system permease 3, glucose dehydrogenase, glucose-1-phosphate cytidylyltransferase, glucose-6-phosphate 1-dehydrogenase, glutamate-ammonia-ligase, glutamine amidotransferase, glutamine synthetase, glutaminyl-tRNA synthetase, glutaredoxin, glutathione S-transferase, glycerol dehydrogenase, glycerol-3-phosphate dehydrogenase, glycogen (starch) synthase, glycogen synthase, glycogenin, glycoprotease, glycosidase, glycosyl transferase, GM02LC11114-protein, GM02LC17485-protein, GM02LC46-protein, GM02LC5744-protein, H/ACA ribonucleoprotein complex subunit 3, harpin-induced family protein, heat shock protein, heat shock transcription factor, HesB/YadR/YfhF family protein, histone H2A, homocitrate synthase, hydrolase, integral membrane transporter family protein, iron(III) dicitrate-binding protein, isochorismate synthase, isocitrate dehydrogenase, isopentenyl diphosphate isomerase, L-asparaginase, L-aspartate oxidase, Leucyl/phenylalanyl-tRNA-protein transferase, lipoprotein precursor, L-ribulose-5-phosphate 4-epimerase, lysyl-tRNA synthetase, major facilitator superfamily transporter protein, malate dehydrogenase, malic enzyme, malonyl CoA-acyl carrier protein transacylase, membrane protein, membrane transport protein, metal-dependent hydrolase, methylglyoxal synthase, methyltransferase, mitochondrial processing protease, molecular chaperone portein, monothiol glutaredoxin, monthiol glutaredoxin, multiple antibiotic resistance protein, murein transglycosylase, N-acetyl-gamma-glutamyl-phosphate reductase, NAD(P)H-quinone oxidoreductase subunit, NADH dehydrogenase I chain I, nitrate/nitrite transport protein, ornithine carbamoyltransferase, oxidoreductase, oxidoreductase subunit, oxireductase, permease protein of phosphate ABC transporter, peroxisome assembly protein, phosphatidylinositol 3- and 4-kinase family protein, phosphoadenosine phosphosulfate reductase, phosphoanhydride phosphorylase, phosphopantetheine adenylyltransferase, phosphopantothenoylcysteine decarboxylase, phosphopantothenoylcysteinesynthetase/decarboxylase, phosphoribosylaminoimidazole carboxylase catalytic subunit, phosphoribosylformyl glycinamidine synthase subunit, phosphoribosylglycinamide formyltransferase, phosphoribosyltransferase, Photosystem I reaction center subunit XI, photosystem II protein, polygalacturonase, polyphosphate kinase, potassium transport protein, pre-mRNA-splicing factor, protease, protein kinase, protein phosphatase, purine nucleoside phosphorylase, purinenucleoside phosphorylase, pyrroline carboxylate reductase, pyruvate kinase, quinolinate synthetase, riboflavin biosynthesis protein, ribosephosphate isomerase, constitutive, RNA binding protein, s_pp015018333r-protein, sec-independent protein translocase, Sec-independent protein translocase subunit, serine protease, serine protease inhibitor, short-chain alcohol dehydrogenase family, sll0064-protein, sll0254-protein, sll0354-protein, sll1761-protein, slr0600-protein, sodium/proton antiporter, squalene monooxygenase, sterol O-acyltransferase, thioredoxin, thioredoxin family protein, threonine dehydrogenase, threonine synthase, transcription factor, transcriptional regulator, transcriptional regulator protein, translation initiation factor subunit, transport protein, TTC0768-protein, TTC1386-protein, urease subunit, uridine/cytidine kinase, valine-pyruvate transaminase, XM_{—}473199-protein, YCL026C-A-protein, ycr102c-protein, ydr338c-protein, yer014w-protein, yer106w-protein, YFL019C-protein, yfl054c-protein, yg1237c-protein, ygr068c-protein, ygr221 c-protein, yhl013c-protein, yhr207c-protein, ylr065c-protein, ylr178c-protein, ynl142w-protein, yor221c-protein, zinc finger protein, zinc transporter, Zm_{—}4842_BE510522-protein, and ZM06LC11975-protein, in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of arginine, glutamate, glutamine or proline, respectively, or a composition comprising arginine, glutamate, glutamine or proline, respectively, in said non-human organism or in the culture medium surrounding said non-human organism.

Item 2. A process for the production of the respective fine chemical arginine, glutamate, glutamine or proline, respectively, which comprises

### (A)

(i) increasing or generating of the expression of; and/or
(ii) increasing or generating the expression of an expression product of; and/or
(iii) increasing or generating one or more activities of an expression product encoded by;
at least one respective nucleic acid molecule comprising a nucleic acid molecule selected from the group consisting of:
(a) a nucleic acid molecule encoding the polypeptide shown in column 5 or 8 of

Table II, preferably Table II B, application no. 1, or a homolog or a fragment thereof, whereby the respective line discloses in column 7 arginine, glutamate, glutamine or proline, respectively;
(b) a nucleic acid molecule shown in column 5 or 8 of Table I, preferably Table I B, application no. 1, or a homolog or a fragment thereof (preferably the coding region thereof), whereby the respective line discloses in column 7 arginine, glutamate, glutamine or proline, respectively;
(c) a nucleic acid molecule, which, as a result of the degeneracy of the genetic code, can be derived from a polypeptide sequence depicted in column 5 or 8 of Table II, preferably Table II B, application no. 1, whereby the respective line discloses in column 7 arginine, glutamate, glutamine or proline, respectively;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 1, or the coding region thereof, whereby the respective line discloses in column 7 arginine, glutamate, glutamine or proline, respectively;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 1, whereby the respective line discloses in column 7 arginine, glutamate, glutamine or proline, respectively;
(i) a nucleic acid molecule encoding a polypeptide having the activity represented by a respective protein as depicted in column 5 of Table II, application no. 1, whereby the respective line discloses in column 7 arginine, glutamate, glutamine or proline, respectively;
(j) a nucleic acid molecule which comprises a polynucleotide, which is obtained by amplifying a cDNA library or a genomic library using the primers as depicted in column 8 of Table III, application no. 1, whereby the respective line discloses in column 7 arginine, glutamate, glutamine or proline, respectively; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
   or a nucleic acid molecule comprising a sequence which is complementary thereto; in a non-human organism or a part thereof, preferably a microorganism, a plant cell, a plant or a part thereof, as compared to a corresponding non-transformed wild type non-human organism or a part thereof; and
   (B) growing the non-human organism or a part thereof under conditions which permit the production of arginine, glutamate, glutamine or proline, respectively, or a composition comprising arginine, glutamate, glutamine or proline, respectively, in said non-human organism or in the culture medium surrounding said non-human organism.

Item 3. A process of items 1 or 2, comprising of recovering the respective fine chemical in its free or bound form.

Item 4. The process of any one of items 1 to 3, comprising the folowing steps:
(a) selecting a non-human organism or a part thereof expressing a polypeptide encoded by the nucleic acid molecule characterized in item 2;
(b) mutagenizing the selected non-human organism or the part thereof;
(c) comparing the activity or the expression level of said polypeptide in the mutagenized non-human organism or the part thereof with the activity or the expression of said polypeptide of the selected non-human organisms or the part thereof;
(d) selecting the mutated non-human organisms or parts thereof, which comprise an increased activity or expression level of said polypeptide compared to the selected non-human organism or the part thereof;
(e) optionally, growing and cultivating the non-human organisms or the parts thereof; and
(f) recovering, and optionally isolating, the free or bound arginine, glutamate, glutamine or proline, respectively, produced by the selected mutated non-human organisms or parts thereof.

Item 5. The process of any one of items 1 to 4 wherein the activity of said protein or the expression of said nucleic acid molecule is increased or generated transiently or stably.

Item 6. An isolated nucleic acid molecule comprising a nucleic acid molecule selected from the group consisting of:
(a) a nucleic acid molecule encoding a polypeptide shown in Table II, application no. 1, column 5 or 8, preferably shown in Table II B, application no. 1, column 8, or a homolog or a fragment thereof;
(b) a nucleic acid molecule shown in Table I, application no. 1, column 5 or 8, preferably shown in Table I B, application no. 1, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 1, preferably in column 8 of Table II B, application no. 1;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 1, preferably shown in column 8 of Table I B, application no. 1, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 1,
(i) a nucleic acid molecule encoding a polypeptide having the activity represented by a respective protein as depicted in column 5 of Table II, application no. 1;
(j) a nucleic acid molecule comprising a polynucleotide which is obtained by amplifying a cDNA library or a genomic library using the primers in Table III, application no. 1, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j) ;
   or a nucleic acid molecule comprising a sequence which is complementary thereto.

Item 7. A nucleic acid construct which confers the expression of the nucleic acid molecule as defined in item 6, comprising one or more regulatory elements.

Item 8. An expression cassette comprising
(a) a promoter, preferably selected from the group consisting of Big35S, PCUbi, Super ans USP;
(b) a nucleic acid molecule as defined in item 6.

Item 9. A vector comprising the nucleic acid molecule as defined in item 6 or the nucleic acid construct as claimed in item 7 or the expression cassette as claimed in item 8.

Item 10. A host cell or a plant organelle, which has been transformed with the vector as claimed in item 9 or the nucleic acid molecule as defined in item 6 or the nucleic acid construct as claimed in item 7 or the expression cassette as claimed in item 8.

Item 11. The host cell of item 10, which is a plant cell or a microorganism.

Item 12. A process for producing a polypeptide, wherein the polypeptide is expressed in a host cell as claimed in item 10 or 11.

Item 13. A polypeptide produced by the process as claimed in item 12 or encoded by the nucleic acid molecule as defined in item 6.

Item 14. An antibody, which binds specifically to the polypeptide as claimed in item 13.

Item 15. A plant tissue, propagation material, harvested material or a plant or s part thereof comprising the host cell as claimed in item 10 or 11.

Item 16. A process for the identification of a compound conferring an increase in arginine, glutamate, glutamine or proline production, respectively, in a non-human organism, comprising the steps:
(a) culturing a plant cell or tissue or microorganism or maintaining a plant expressing the polypeptide encoded by the nucleic acid molecule as defined in item 6 conferring an increase in the amount of arginine, glutamate, glutamine or proline, respectively, in a non-human organism or a part thereof and a readout system capable of interacting with the polypeptide under suitable conditions which permit the interaction of the polypeptide with said readout system in the presence of a compound or a sample comprising a plurality of compounds and capable of providing a detectable signal in response to the binding of a compound to said polypeptide under conditions which permit the expression of said readout system and of the polypeptide encoded by the nucleic acid molecule of item 6 conferring an increase in the amount of arginine, glutamate, glutamine or proline, respectively, in a non-human organism or a part thereof;
(b) identifying if the compound is an effective agonist by detecting the presence or absence or increase of a signal produced by said readout system.

Item 17. A method for the production of an agricultural composition comprising the steps of the method of item 16 and formulating the compound identified in item 16 in a form acceptable for an application in agriculture.

Item 18. A composition comprising the nucleic acid molecule as defined in item 6, the polypeptide as claimed in item 13, the nucleic acid construct as claimed in item 7, the expression cassette as claimed in item 8, the vector as claimed in item 9, the compound as claimed in item 16, the antibody as claimed in item 14, and optionally an agricultural acceptable carrier.

Item 19. Use of the nucleic acid molecule as defined in item 6 for the identification of a nucleic acid molecule conferring an increase in arginine, glutamate, glutamine or proline, respectively, after expression.

Item 20. Cosmetic, pharmaceutical, food or feed composition comprising the nucleic acid moleculeas defined in item 6, the polypeptide as claimed in item 13, the nucleic acid construct as claimed in item 7, the expression cassette as claimed in item 8, the vector as claimed in item 9, the antibody as claimed in item 14 , the plant or a part thereof, or plant tissue as claimed in item 15, the harvested material or propagation material as claimed in item 15 or the host cell as claimed in items 10 or 11.

Item 21. Use of the nucleic acid molecule as defined in item 6, the polypeptide as claimed in item 13, the nucleic acid construct as claimed in item 7, the expression cassette as claimed in item 8, the vector as claimed in item 9, the plant or plant tissue as claimed in item 15, or the host cell as claimed initem 10 to 11 for the production of plant resistant to a herbicide inhibiting the production of arginine, glutamate, glutamine or proline.

Item 22. A method for producing a plant with increased yield as compared to a corresponding non-transformed wild type plant, whereby the method comprises
(i) increasing or generating of the expression of; and/or
(ii) increasing or generating the expression of an expression product of; and/or
(iii) increasing or generating one or more activities of an expression product encoded by; at least one respective nucleic acid molecule comprising a nucleic acid molecule selected from the group consisting of:
(a) a nucleic acid molecule encoding the polypeptide shown in column 5 or 8 of Table II, preferably Table II B, application no. 7, or a homolog or a fragment thereof, in case in column 7 arginine, glutamate, glutamine or proline, respectively is indicated;
(b) a nucleic acid molecule shown in column 5 or 8 of Table I, preferably Table I B, application no. 7, or a homolog or a fragment thereof (preferably the coding region thereof) , in case in column 7 arginine, glutamate, glutamine or proline, respectively, is indicated;
(c) a nucleic acid molecule, which, as a result of the degeneracy of the genetic code, can be derived from a polypeptide sequence depicted in column 5 or 8 of Table II, preferably Table II B, application no. 7, in case in column 7 arginine, glutamate, glutamine or proline, respectively, is indicated;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 7, or the coding region thereof, in case in column 7 arginine, glutamate, glutamine or proline , respectively, is indicated;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 7, in case in column 7 arginine, glutamate, glutamine or proline, respectively, is indicated;
(i) a nucleic acid molecule encoding a polypeptide having the activity represented by a respective protein as depicted in column 5 of Table II, application no. 7, in case in column 7 arginine, glutamate, glutamine or proline, respectively, is indicated;
(j) a nucleic acid molecule which comprises a polynucleotide, which is obtained by amplifying a cDNA library or a genomic library using the primers as depicted in column 8 of Table III, application no. 7, in case in column 7 arginine, glutamate, glutamine or proline , respectively, is indicated; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
   or a nucleic acid molecule comprising a sequence which is complementary thereto; in a plant cell, plant or a part thereof.

Item 23. A method for producing a plant with increased yield according to item 22, which comprises in addition the regeneration of a plant from such transformed plant cell, plant cell compartment, plant cell nucleus, plant cell or plant tissue with increased yield.

[0483.1.1.2] to [0494.1.1.2] for the disclosure of these paragraphs see [0483.1.1.1] to [0494.1.1.1] above.

### Example 11a

### PCR Amplification of the Sequences

Unless otherwise specified, standard methods as described in Sambrook et al., Molecular Cloning: A laboratory manual, Cold Spring Harbor 1989, Cold Spring Harbor Laboratory Press are used.

The inventive sequences as shown in the respective line in Table I, column 5, prefareably the coding region thereof, were amplified by PCR as described in the protocol of the Pfu Ultra, Pfu Turbo or Herculase DNA polymerase (Stratagene). The composition for the protocol of the Pfu

Ultra, Pfu Turbo or Herculase DNA polymerase was as follows: 1×PCR buffer (Stratagene), 0.2 mM of each dNTP, 100 ng genomic DNA of Saccharomyces cerevisiae (strain S288C; Research Genetics, Inc., now Invitrogen), Escherichia coli (strain MG1655; E. coli Genetic Stock Center), Synechocystis sp. (strain PCC6803), Azotobacter vinelandii (strain N.R. Smith,16), Thermus thermophilus (HB8) or 50 ng cDNA from various tissues and development stages of Arabidopsis thaliana (ecotype Columbia), Physcomitrella patens, Glycine max (variety Resnick), Brassica napus, Oryza sativa or Zea mays (variety B73, Mo17, A188), 50 pmol forward primer, 50 pmol reverse primer, with or without 1 M Betaine, 2.5 u Pfu Ultra, Pfu Turbo or Herculase DNA polymerase.

The amplification cycles were as follows:

1 cycle of 2-3 minutes at 94-95° C., then 25-36 cycles with 30-60 seconds at 94-95° C., 30-45 seconds at 50-60° C. and 210-480 seconds at 72° C., followed by 1 cycle of 5-10 minutes at 72° C., then 4-16° C.—preferably for Saccharomyces cerevisiae, Escherichia coli, Synechocystis sp., Azotobacter vinelandii, Thermus thermophilus.

In case of Arabidopsis thaliana, Brassica napus, Glycine max, Oryza sativa, Physcomitrella patens, Zea mays the amplification cycles were as follows:

1 cycle with 30 seconds at 94° C., 30 seconds at 61° C., 15 minutes at 72° C., then 2 cycles with 30 seconds at 94° C., 30 seconds at 60° C., 15 minutes at 72° C., then 3 cycles with 30 seconds at 94° C., 30 seconds at 59° C., 15 minutes at 72° C., then 4 cycles with 30 seconds at 94° C., 30 seconds at 58° C., 15 minutes at 72° C., then 25 cycles with 30 seconds at 94° C., 30 seconds at 57° C., 15 minutes at 72° C., then 1 cycle with 10 minutes at 72° C., then finally 4-16° C.

RNAs were generated with the RNeasy Plant Kit according to the standard protocol (Qiagen) and Superscript II Reverse Transkriptase was used to produce double stranded cDNA according to the standard protocol (Invitrogen).

ORF specific primer pairs for the genes to be expressed are shown in the respective line in Table III, column 8. The following adapter sequences were added to Saccharomyces cerevisiae ORF specific primers (see Table III) for cloning purposes:

SEQ ID NO: 20

| | | |
|---|---|---|
| i) | foward primer: | 5′-GGAATTCCAGCTGACCACC-3′ |

SEQ ID NO: 21

| | | |
|---|---|---|
| ii) | reverse primer: | 5′-GATCCCCGGGAATTGCCATG-3′ |

These adaptor sequences allow cloning of the ORF into the various vectors containing the Resgen adaptors, see table column E of Table c.

The following adapter sequences were added to Saccharomyces cerevisiae, Escherichia coli, Synechocystis sp., Azotobacter vinelandii, Thermus thermophilus, Arabidopsis thaliana, Brassica napus, Glycine max, Oryza sativa, Physcomitrella patens, or Zea mays ORF specific primers for cloning purposes:

| SEQ ID NO: 22 | | |
|---|---|---|
| iii) | forward primer: | 5′-TTGCTCTTCC-3′ |
| SEQ ID NO: 23 | | |
| iiii) | reverse primer: | 5′-TTGCTCTTCG-3′ |

The adaptor sequences allow cloning of the ORF into the various vectors containing the Colic adaptors, see table column E of Table c.

Therefore for amplification and cloning of Saccharomyces cerevisiae SEQ ID NO: 14302, a primer consisting of the adaptor sequence i) and the ORF specific sequence SEQ ID NO: 14696 and a second primer consisting of the adaptor sequence ii) and the ORF specific sequence SEQ ID NO: 14697 were used.

For amplification and cloning of Escherichia coli SEQ ID NO: 7081, a primer consisting of the adaptor sequence iii) and the ORF specific sequence SEQ ID NO: 7261 and a second primer consisting of the adaptor sequence iiii) and the ORF specific sequence SEQ ID NO: 7262 were used.

For amplification and cloning of Synechocystis sp. SEQ ID NO: 11423, a primer consisting of the adaptor sequence iii) and the ORF specific sequence SEQ ID NO: 11465 and a second primer consisting of the adaptor sequence iiii) and the ORF specific sequence SEQ ID NO: 11466 were used.

For amplification and cloning of Arabidopsis thaliana SEQ ID NO: 1061, a primer consisting of the adaptor sequence iii) and the ORF specific sequence SEQ ID NO: 1293 and a second primer consisting of the adaptor sequence iiii) and the ORF specific sequence SEQ ID NO:

1294 were used.

For amplification and cloning of Azotobacter vinelandii SEQ ID NO: 5557, a primer consisting of the adaptor sequence iii) and the ORF specific sequence SEQ ID NO: 6033 and a second primer consisting of the adaptor sequence iiii) and the ORF specific sequence SEQ ID NO: 6034 were used.

For amplification and cloning of Brassica napus SEQ ID NO: 15532, a primer consisting of the adaptor sequence iii) and the ORF specific sequence SEQ ID NO: 16150 and a second primer consisting of the adaptor sequence iiii) and the ORF specific sequence SEQ ID NO: 16151 were used.

For amplification and cloning of Glycine max SEQ ID NO: 69, a primer consisting of the adaptor sequence iii) and the ORF specific sequence SEQ ID NO: 343 and a second primer consisting of the adaptor sequence iiii) and the ORF specific sequence SEQ ID NO: 344 were used.

For amplification and cloning of Oryza sativa SEQ ID NO: 34301, a primer consisting of the adaptor sequence iii) and the ORF specific sequence SEQ ID NO: 34595 and a second primer consisting of the adaptor sequence iiii) and the ORF specific sequence SEQ ID NO: 34596 were used.

For amplification and cloning of Physcomytrella patens SEQ ID NO: 45757, a primer consisting of the adaptor sequence iii) and the ORF specific sequence SEQ ID NO: 45791 and a second primer consisting of the adaptor sequence iiii) and the ORF specific sequence SEQ ID NO: 45792 were used.

For amplification and cloning of Thermus thermophilus SEQ ID NO: 12698, a primer consisting of the adaptor sequence iii) and the ORF specific sequence SEQ ID NO: 12970 and a second primer consisting of the adaptor sequence iiii) and the ORF specific sequence SEQ ID NO: 12971 were used.

For amplification and cloning of Zea mays SEQ ID NO: 68132, a primer consisting of the adaptor sequence iii) and the ORF specific sequence SEQ ID NO: 68358 and a second primer consisting of the adaptor sequence iiii) and the ORF specific sequence SEQ ID NO: 68359 were used.

Following these examples every sequence disclosed in Table I, preferably column 5, especially the coding region thereof can be cloned by fusing the adaptor sequences to the respective specific primers sequences as disclosed in Table III, column 8 using the respective vectors shown in Table c.

[0496.1.1.2] to [0499.1.1.2] for the disclosure of these paragraphs see [0496.1.1.1] to [0499.1.1.1] above.

### Example 11e

### Cloning of Inventive Sequences as Shown in Table I, Column 5 in the Different Expression Vectors

For cloning of for example SEQ ID NO: 7081 from Escherichia coli or any other ORF from Escherichia coli, Synechocystis sp., Azotobacter vinelandii, Thermus thermophilus, Arabidopsis thaliana, Brassica napus, Glycine max, Oryza sativa, Physcomitrella patens, or Zea mays the vector DNA was treated with the restriction enzymes Pacl and Ncol following the standard protocol (MBI Fermentas).

For cloning for example the ORFs of SEQ ID NO: 66772 from Saccharomyces cerevisiae or any other ORF from Saccharomyces cerevisiae into vectors containing the Resgen adaptor sequence the respective vector DNA was treated with the restriction enzyme Ncol.

For cloning of for example ORFs SEQ ID NO: 66419 from Saccharomyces cerevisiae or any other ORF from Saccharomyces cerevisiae into vectors containing the Colic adaptor sequence, the respective vector DNA was treated with the restriction enzymes Pacl and Ncol following the standard protocol (MBI Fermentas).

In all cases the reaction was stopped by inactivation at 70° C. for 20 minutes and purified over QlAquick or NucleoSpin Extract II columns following the standard protocol (Qiagen or Macherey-Nagel).

Then the PCR-product representing the amplified ORF with the respective adapter sequences and the vector DNA were treated with T4 DNA polymerase according to the standard protocol (MBI Fermentas) to produce single stranded overhangs with the parameters 1 unit T4 DNA polymerase at 37° C. for 2-10 minutes for the vector and 1-2 u T4 DNA polymerase at 15-17° C. for 10-60 minutes for the PCR product representing SEQ ID NO: 7081.

The reaction was stopped by addition of high-salt buffer and purified over QlAquick or NucleoSpin Extract II columns following the standard protocol (Qiagen or Macherey-Nagel).

According to this example the skilled person is able to clone all sequences disclosed in Table I, preferably column 5 or column 8, especially the coding region thereof.

Approximately 30-60 ng of prepared vector and a defined amount of prepared amplificate were mixed and hybridized at 65° C. for 15 minutes followed by 37° C. 0,1 ° C./1 seconds, followed by 37° C. 10 minutes, followed by 0,1 ° C./1 seconds, then 4-10 ° C.

The ligated constructs were transformed in the same reaction vessel by addition of competent E. coli cells (strain DHSalpha) and incubation for 20 minutes at 1° C. followed by a heat shock for 90 seconds at 42° C. and cooling to 1-4° C. Then, complete medium (SOC) was added and the mixture was incubated for 45 minutes at 37° C. The entire mixture was subsequently plated onto an agar plate with 0.05 mg/ml kanamycin and incubated overnight at 37° C.

The outcome of the cloning step was verified by amplification with the aid of primers which bind upstream and downstream of the integration site, thus allowing the amplification of the insertion.

The amplifications were carried out as described in the protocol of Taq DNA polymerase (Gibco-BRL).

The amplification cycles were as follows:

1 cycle of 1-5 minutes at 94° C., followed by 35 cycles of in each case 15-60 seconds at 94° C., 15-60 seconds at 50-66° C. and 5-15 minutes at 72° C., followed by 1 cycle of 10 minutes at 72° C., then 4-16° C.

Several colonies were checked, but only one colony for which a PCR product of the expected size was detected was used in the following steps.

A portion of this positive colony was transferred into a reaction vessel filled with complete medium (LB) supplemented with kanamycin and incubated overnight at 37° C.

The plasmid preparation was carried out as specified in the Qiaprep or NucleoSpin Multi-96 Plus standard protocol (Qiagen or Macherey-Nagel).

[0501.1.1.2] to [0503.1.1.2] for the disclosure of these paragraphs see [0501.1.1.1] to [0503.1.1.1] above.

### Table d Showing Results of Plant Analyses

TABLE d

| SeqID | Target | Locus | Metabolite | Source | Promotor | Method | Min | Max |
|---|---|---|---|---|---|---|---|---|
| 15187 | non- | 47266012_SOYBEAN | glutamate | ARA_LEAF | p-PcUBI | GC | 42 | 96 |
| | targeted | | | | | | | |
| 15187 | non- | 47266012_SOYBEAN | glutamine | ARA_LEAF | p-PcUBI | GC | 72 | 234 |
| | targeted | | | | | | | |
| 69 | non- | 49747384_SOYBEAN | glutamate | ARA_LEAF | p-PcUBI | GC | 37 | 122 |
| | targeted | | | | | | | |
| 15532 | plastidic | 51340801_CANOLA | glutamate | ARA_LEAF | p-PcUBI | LC | 35 | 135 |
| 15532 | plastidic | 51340801_CANOLA | proline | ARA_LEAF | p-PcUBI | GC | 38 | 189 |
| 16155 | non- | 59547452_SOYBEAN | arginine | ARA_LEAF | p-PcUBI | LC | 65 | 457 |
| | targeted | | | | | | | |
| 16263 | plastidic | 59554615_SOYBEAN | glutamate | ARA_LEAF | p-PcUBI | LC | 44 | 80 |
| 16883 | plastidic | 59582753_SOYBEAN | arginine | ARA_LEAF | p-PcUBI | LC | 66 | 241 |
| 16883 | plastidic | 59582753_SOYBEAN | proline | ARA_LEAF | p-PcUBI | GC | 34 | 204 |
| 17356 | plastidic | AAC43185 | arginine | ARA_LEAF | p-PcUBI | LC | 586 | 3134 |
| 17451 | non- | At1g07430 | glutamate | ARA_LEAF | p-PcUBI | LC | 39 | 67 |
| | targeted | | | | | | | |
| 17451 | non- | At1g07430 | glutamine | ARA_LEAF | p-PcUBI | LC | 27 | 35 |
| | targeted | | | | | | | |
| 17601 | non- | At1g17440 | arginine | ARA_LEAF | p-PcUBI | LC | 54 | 181 |
| | targeted | | | | | | | |
| 17637 | non- | At1g19800 | glutamine | ARA_LEAF | p-PcUBI | GC | 53 | 175 |
| | targeted | | | | | | | |
| 17701 | non- | At1g26830 | arginine | ARA_LEAF | p-PcUBI | LC | 64 | 354 |
| | targeted | | | | | | | |
| 17701 | non- | At1g26830 | proline | ARA_LEAF | p-PcUBI | GC | 65 | 193 |
| | targeted | | | | | | | |
| 17901 | non- | At1g29350 | arginine | ARA_LEAF | p-PcUBI | LC | 51 | 249 |
| | targeted | | | | | | | |
| 17968 | non- | At1g36730 | arginine | ARA_LEAF | p-PcUBI | LC | 56 | 193 |
| | targeted | | | | | | | |
| 17968 | non- | At1g36730 | glutamine | ARA_LEAF | p-PcUBI | GC | 48 | 136 |
| | targeted | | | | | | | |
| 18070 | non- | At1g43850 | arginine | ARA_LEAF | p-PcUBI | LC | 49 | 294 |
| | targeted | | | | | | | |
| 18070 | non- | At1g43850 | glutamate | ARA_LEAF | p-PcUBI | GC | 54 | 158 |
| | targeted | | | | | | | |
| 18070 | non- | At1g43850 | glutamine | ARA_LEAF | p-PcUBI | GC | 42 | 488 |
| | targeted | | | | | | | |
| 18070 | non- | At1g43850 | proline | ARA_LEAF | p-PcUBI | GC | 38 | 520 |
| | targeted | | | | | | | |
| 18122 | non- | At1g47380 | proline | ARA_LEAF | p-PcUBI | GC | 39 | 237 |
| | targeted | | | | | | | |
| 18235 | non- | At1g48260 | arginine | ARA_LEAF | p-PcUBI | LC | 70 | 338 |
| | targeted | | | | | | | |
| 18235 | non- | At1g48260 | glutamate | ARA_LEAF | p-PcUBI | GC | 53 | 362 |
| | targeted | | | | | | | |
| 18235 | non- | At1g48260 | proline | ARA_LEAF | p-PcUBI | GC | 34 | 108 |
| | targeted | | | | | | | |
| 18869 | non- | At1g61950 | arginine | ARA_LEAF | p-PcUBI | LC | 89 | 385 |
| | targeted | | | | | | | |
| 18869 | non- | At1g61950 | glutamine | ARA_LEAF | p-PcUBI | GC | 54 | 319 |
| | targeted | | | | | | | |
| 19364 | non- | At1g67340 | proline | ARA_LEAF | p-PcUBI | GC | 36 | 86 |
| | targeted | | | | | | | |
| 1061 | non- | At1g68320 | glutamine | ARA_LEAF | p-PcUBI | GC | 57 | 183 |
| | targeted | | | | | | | |
| 19419 | non- | At1g72770 | glutamine | ARA_LEAF | p-PcUBI | GC | 44 | 290 |
| | targeted | | | | | | | |
| 19502 | non- | At2g17560 | proline | ARA_LEAF | p-PcUBI | GC | 39 | 181 |
| | targeted | | | | | | | |
| 1298 | non- | At2g25070 | glutamate | ARA_LEAF | p-PcUBI | LC | 33 | 77 |
| | targeted | | | | | | | |
| 1298 | non- | At2g25070 | glutamine | ARA_LEAF | p-PcUBI | GC | 60 | 213 |
| | targeted | | | | | | | |
| 19671 | non- | At2g26390 | proline | ARA_LEAF | p-PcUBI | GC | 38 | 120 |
| | targeted | | | | | | | |
| 19874 | non- | At2g28890 | proline | ARA_LEAF | p-PcUBI | GC | 36 | 716 |
| | targeted | | | | | | | |
| 19919 | non- | At2g30360 | glutamine | ARA_LEAF | p-PcUBI | GC | 46 | 170 |
| | targeted | | | | | | | |
| 19919 | non- | At2g30360 | proline | ARA_LEAF | p-PcUBI | LC | 43 | 132 |
| | targeted | | | | | | | |
| 20346 | non- | At2g30540 | arginine | ARA_LEAF | p-PcUBI | LC | 62 | 285 |
| | targeted | | | | | | | |
| 20346 | non- | At2g30540 | proline | ARA_LEAF | p-PcUBI | GC | 71 | 1094 |
| | targeted | | | | | | | |
| 20578 | non- | At2g34180 | proline | ARA_LEAF | p-PcUBI | GC | 37 | 136 |
| | targeted | | | | | | | |
| 21008 | non- | At2g39800 | proline | ARA_LEAF | p-PcUBI | GC | 106 | 4073 |
| | targeted | | | | | | | |
| 21106 | non- | At2g46500 | glutamine | ARA_LEAF | p-PcUBI | GC | 44 | 135 |
| | targeted | | | | | | | |
| 21159 | non- | At2g47880 | arginine | ARA_LEAF | p-PcUBI | LC | 253 | 1026 |
| | targeted | | | | | | | |
| 21159 | non- | At2g47880 | glutamine | ARA_LEAF | p-PcUBI | LC | 90 | 179 |
| | targeted | | | | | | | |
| 21159 | non- | At2g47880 | proline | ARA_LEAF | p-PcUBI | LC | 49 | 717 |
| | targeted | | | | | | | |
| 21497 | plastidic | At3g04050 | glutamate | ARA_LEAF | p-PcUBI | GC | 36 | 311 |
| 21497 | plastidic | At3g04050 | proline | ARA_LEAF | p-PcUBI | GC | 33 | 272 |
| 21902 | non- | At3g04710 | proline | ARA_LEAF | p-PcUBI | GC | 35 | 230 |
| | targeted | | | | | | | |
| 22015 | non- | At3g06270 | glutamate | ARA_LEAF | p-PcUBI | GC | 36 | 99 |
| | targeted | | | | | | | |
| 22249 | non- | At3g08710 | arginine | ARA_LEAF | p-PcUBI | LC | 56 | 513 |
| | targeted | | | | | | | |
| 22249 | non- | At3g08710 | glutamate | ARA_LEAF | p-PcUBI | GC | 45 | 167 |
| | targeted | | | | | | | |
| 22249 | non- | At3g08710 | glutamine | ARA_LEAF | p-PcUBI | GC | 76 | 558 |
| | targeted | | | | | | | |
| 22249 | non- | At3g08710 | proline | ARA_LEAF | p-PcUBI | GC | 70 | 169 |
| | targeted | | | | | | | |
| 22611 | non- | At3g11650 | arginine | ARA_LEAF | p-PcUBI | LC | 46 | 397 |
| | targeted | | | | | | | |
| 22611 | non- | At3g11650 | glutamine | ARA_LEAF | p-PcUBI | GC | 44 | 124 |
| | targeted | | | | | | | |
| 22611 | non- | At3g11650 | proline | ARA_LEAF | p-PcUBI | LC | 50 | 129 |
| | targeted | | | | | | | |
| 22699 | plastidic | At3g14230 | proline | ARA_LEAF | p-PcUBI | GC | 53 | 142 |
| 22832 | non- | At3g18524 | proline | ARA_LEAF | p-PcUBI | GC | 38 | 75 |
| | targeted | | | | | | | |
| 22921 | non- | At3g20910 | proline | ARA_LEAF | p-PcUBI | GC | 61 | 204 |
| | targeted | | | | | | | |
| 1815 | non- | At3g23000 | glutamine | ARA_LEAF | p-PcUBI | GC | 79 | 288 |
| | targeted | | | | | | | |
| 23002 | non- | At3g27300 | proline | ARA_LEAF | p-PcUBI | GC | 38 | 123 |
| | targeted | | | | | | | |
| 2573 | non- | At3g62930 | glutamate | ARA_LEAF | p-PcUBI | GC | 39 | 132 |
| | targeted | | | | | | | |
| 2573 | non- | At3g62930 | glutamine | ARA_LEAF | p-PcUBI | GC | 51 | 277 |
| | targeted | | | | | | | |
| 2935 | non- | At3g62950 | glutamate | ARA_LEAF | p-PcUBI | LC | 33 | 89 |
| | targeted | | | | | | | |
| 2935 | non- | At3g62950 | glutamine | ARA_LEAF | p-PcUBI | GC | 127 | 250 |
| | targeted | | | | | | | |
| 23482 | non- | At4g15660 | glutamate | ARA_LEAF | p-PcUBI | GC | 38 | 164 |
| | targeted | | | | | | | |
| 23482 | non- | At4g15660 | glutamine | ARA_LEAF | p-PcUBI | GC | 44 | 116 |
| | targeted | | | | | | | |
| 3279 | non- | At4g15670 | glutamate | ARA_LEAF | p-PcUBI | GC | 77 | 165 |
| | targeted | | | | | | | |
| 23844 | non- | At4g15690 | glutamate | ARA_LEAF | p-PcUBI | GC | 47 | 145 |
| | targeted | | | | | | | |
| 23844 | non- | At4g15690 | glutamine | ARA_LEAF | p-PcUBI | GC | 53 | 270 |
| | targeted | | | | | | | |
| 23844 | non- | At4g15690 | proline | ARA_LEAF | p-PcUBI | LC | 44 | 156 |
| | targeted | | | | | | | |
| 3654 | non- | At4g15700 | glutamine | ARA_LEAF | p-PcUBI | GC | 55 | 267 |
| | targeted | | | | | | | |
| 24232 | non- | At4g18880 | proline | ARA_LEAF | p-PcUBI | GC | 46 | 216 |
| | targeted | | | | | | | |
| 4040 | non- | At4g32480 | arginine | ARA_LEAF | p-PcUBI | LC | 52 | 215 |
| | targeted | | | | | | | |
| 4040 | non- | At4g32480 | glutamine | ARA_LEAF | p-PcUBI | GC | 301 | 4258 |
| | targeted | | | | | | | |
| 4040 | non- | At4g32480 | proline | ARA_LEAF | p-PcUBI | GC | 45 | 111 |
| | targeted | | | | | | | |
| 4102 | non- | At4g33040 | glutamate | ARA_LEAF | p-PcUBI | GC | 46 | 104 |
| | targeted | | | | | | | |
| 24311 | non- | At4g34160 | arginine | ARA_LEAF | p-PcUBI | LC | 90 | 236 |
| | targeted | | | | | | | |
| 24311 | non- | At4g34160 | glutamine | ARA_LEAF | p-PcUBI | GC | 51 | 313 |
| | targeted | | | | | | | |
| 24311 | non- | At4g34160 | proline | ARA_LEAF | p-PcUBI | GC | 49 | 772 |
| | targeted | | | | | | | |
| 4348 | non- | At4g35310 | arginine | ARA_LEAF | p-PcUBI | LC | 58 | 499 |
| | targeted | | | | | | | |
| 4348 | non- | At4g35310 | proline | ARA_LEAF | p-PcUBI | GC | 126 | 329 |
| | targeted | | | | | | | |
| 24438 | non- | At5g03720 | glutamate | ARA_LEAF | p-PcUBI | GC | 67 | 186 |
| | targeted | | | | | | | |
| 24492 | non- | At5g07200 | proline | ARA_LEAF | p-PcUBI | LC | 44 | 99 |
| | targeted | | | | | | | |
| 25222 | non- | At5g10820 | glutamate | ARA_LEAF | p-PcUBI | LC | 34 | 69 |
| | targeted | | | | | | | |
| 25222 | non- | At5g10820 | glutamine | ARA_LEAF | p-PcUBI | GC | 45 | 139 |
| | targeted | | | | | | | |
| 25283 | non- | At5g16650 | arginine | ARA_LEAF | p-PcUBI | LC | 104 | 361 |
| | targeted | | | | | | | |
| 25283 | non- | At5g16650 | glutamine | ARA_LEAF | p-PcUBI | GC | 48 | 238 |
| | targeted | | | | | | | |
| 25283 | non- | At5g16650 | proline | ARA_LEAF | p-PcUBI | GC | 41 | 177 |
| | targeted | | | | | | | |
| 4904 | non- | At5g18600 | glutamine | ARA_LEAF | p-PcUBI | GC | 82 | 254 |
| | targeted | | | | | | | |
| 25344 | non- | At5g27640 | arginine | ARA_LEAF | p-PcUBI | LC | 67 | 139 |
| | targeted | | | | | | | |
| 25428 | non- | At5g39760 | glutamate | ARA_LEAF | p-PcUBI | LC | 40 | 78 |
| | targeted | | | | | | | |
| 25428 | non- | At5g39760 | proline | ARA_LEAF | p-PcUBI | GC | 32 | 275 |
| | targeted | | | | | | | |
| 5318 | non- | At5g57050 | glutamine | ARA_LEAF | p-PcUBI | LC | 34 | 63 |
| | targeted | | | | | | | |
| 25498 | non- | At5g59220 | glutamine | ARA_LEAF | p-PcUBI | LC | 32 | 52 |
| | targeted | | | | | | | |
| 5493 | non- | At5g64920 | arginine | ARA_LEAF | p-PcUBI | LC | 53 | 291 |
| | targeted | | | | | | | |
| 5493 | non- | At5g64920 | glutamine | ARA_LEAF | p-PcUBI | GC | 52 | 199 |
| | targeted | | | | | | | |
| 25676 | non- | Avin- | glutamate | ARA_LEAF | p-PcUBI | GC | 55 | 90 |
| | targeted | DRAFT_1045 | | | | | | |
| 25780 | non- | Avin- | glutamate | ARA_LEAF | p-PcUBI | LC | 42 | 44 |
| | targeted | DRAFT_1398 | | | | | | |
| 25780 | non- | Avin- | glutamine | ARA_LEAF | p-PcUBI | GC | 56 | 79 |
| | targeted | DRAFT_1398 | | | | | | |
| 5557 | non- | Avin- | glutamine | ARA_LEAF | p-PcUBI | GC | 54 | 292 |
| | targeted | DRAFT_1495 | | | | | | |
| 5557 | non- | Avin- | proline | ARA_LEAF | p-PcUBI | GC | 111 | 227 |
| | targeted | DRAFT_1495 | | | | | | |
| 26120 | non- | Avin- | proline | ARA_LEAF | p-PcUBI | LC | 44 | 119 |
| | targeted | DRAFT_1534 | | | | | | |
| 26196 | non- | Avin- | proline | ARA_LEAF | p-PcUBI | GC | 35 | 78 |
| | targeted | DRAFT_1624 | | | | | | |
| 26434 | non- | Avin- | glutamate | ARA_LEAF | p-PcUBI | LC | 76 | 95 |
| | targeted | DRAFT_1806 | | | | | | |
| 6040 | non- | Avin- | arginine | ARA_LEAF | p-PcUBI | LC | 64 | 530 |
| | targeted | DRAFT_2091 | | | | | | |
| 27021 | non- | Avin- | arginine | ARA_LEAF | p-PcUBI | LC | 115 | 495 |
| | targeted | DRAFT_2344 | | | | | | |
| 27021 | non- | Avin- | glutamate | ARA_LEAF | p-PcUBI | GC | 44 | 145 |
| | targeted | DRAFT_2344 | | | | | | |
| 27021 | non- | Avin- | glutamine | ARA_LEAF | p-PcUBI | GC | 76 | 770 |
| | targeted | DRAFT_2344 | | | | | | |
| 27882 | non- | Avin- | glutamine | ARA_LEAF | p-PcUBI | GC | 58 | 197 |
| | targeted | DRAFT_2521 | | | | | | |
| 27882 | non- | Avin- | proline | ARA_LEAF | p-PcUBI | GC | 39 | 466 |
| | targeted | DRAFT_2521 | | | | | | |
| 28040 | non- | Avin- | arginine | ARA_LEAF | p-PcUBI | LC | 73 | 227 |
| | targeted | DRAFT_2754 | | | | | | |
| 28040 | non- | Avin- | glutamate | ARA_LEAF | p-PcUBI | GC | 38 | 297 |
| | targeted | DRAFT_2754 | | | | | | |
| 28040 | non- | Avin- | glutamine | ARA_LEAF | p-PcUBI | GC | 80 | 466 |
| | targeted | DRAFT_2754 | | | | | | |
| 28040 | non- | Avin- | proline | ARA_LEAF | p-PcUBI | GC | 36 | 170 |
| | targeted | DRAFT_2754 | | | | | | |
| 6075 | non- | Avin- | glutamate | ARA_LEAF | p-PcUBI | GC | 64 | 185 |
| | targeted | DRAFT_3028 | | | | | | |
| 28738 | non- | Avin- | arginine | ARA_LEAF | p-PcUBI | LC | 64 | 144 |
| | targeted | DRAFT_3159 | | | | | | |
| 28738 | non- | Avin- | proline | ARA_LEAF | p-PcUBI | GC | 63 | 531 |
| | targeted | DRAFT_3159 | | | | | | |
| 29246 | non- | Avin- | glutamate | ARA_LEAF | p-PcUBI | GC | 38 | 91 |
| | targeted | DRAFT_3186 | | | | | | |
| 29286 | non- | Avin- | proline | ARA_LEAF | p-PcUBI | LC | 105 | 202 |
| | targeted | DRAFT_3209 | | | | | | |
| 29397 | non- | Avin- | proline | ARA_LEAF | p-PcUBI | GC | 50 | 405 |
| | targeted | DRAFT_3250 | | | | | | |
| 29500 | non- | Avin- | glutamate | ARA_LEAF | p-PcUBI | LC | 34 | 63 |
| | targeted | DRAFT_3253 | | | | | | |
| 29500 | non- | Avin- | glutamine | ARA_LEAF | p-PcUBI | GC | 53 | 128 |
| | targeted | DRAFT_3253 | | | | | | |
| 30039 | non- | Avin- | glutamine | ARA_LEAF | p-PcUBI | LC | 37 | 65 |
| | targeted | DRAFT_3556 | | | | | | |
| 30464 | non- | Avin- | glutamate | ARA_LEAF | p-PcUBI | GC | 36 | 71 |
| | targeted | DRAFT_3587 | | | | | | |
| 31026 | non- | Avin- | glutamate | ARA_LEAF | p-PcUBI | LC | 35 | 57 |
| | targeted | DRAFT_3605 | | | | | | |
| 31026 | non- | Avin- | glutamine | ARA_LEAF | p-PcUBI | GC | 60 | 275 |
| | targeted | DRAFT_3605 | | | | | | |
| 31717 | non- | Avin- | glutamate | ARA_LEAF | p-PcUBI | GC | 35 | 98 |
| | targeted | DRAFT_4384 | | | | | | |
| 31717 | non- | Avin- | glutamine | ARA_LEAF | p-PcUBI | GC | 75 | 196 |
| | targeted | DRAFT_4384 | | | | | | |
| 31717 | non- | Avin- | proline | ARA_LEAF | p-PcUBI | GC | 49 | 109 |
| | targeted | DRAFT_4384 | | | | | | |
| 31926 | non- | Avin- | arginine | ARA_LEAF | p-PcUBI | LC | 58 | 219 |
| | targeted | DRAFT_4562 | | | | | | |
| 6510 | non- | Avin- | proline | ARA_LEAF | p-PcUBI | LC | 66 | 289 |
| | targeted | DRAFT_5103 | | | | | | |
| 32037 | non- | Avin- | arginine | ARA_LEAF | p-PcUBI | LC | 71 | 419 |
| | targeted | DRAFT_5246 | | | | | | |
| 32037 | non- | Avin- | glutamine | ARA_LEAF | p-PcUBI | GC | 70 | 363 |
| | targeted | DRAFT_5246 | | | | | | |
| 32037 | non- | Avin- | proline | ARA_LEAF | p-PcUBI | GC | 69 | 218 |
| | targeted | DRAFT_5246 | | | | | | |
| 32308 | non- | Avin- | proline | ARA_LEAF | p-PcUBI | LC | 47 | 172 |
| | targeted | DRAFT_5292 | | | | | | |
| 32648 | non- | Avin- | arginine | ARA_LEAF | p-PcUBI | LC | 49 | 171 |
| | targeted | DRAFT_5467 | | | | | | |
| 32648 | non- | Avin- | glutamine | ARA_LEAF | p-PcUBI | GC | 45 | 123 |
| | targeted | DRAFT_5467 | | | | | | |
| 32648 | non- | Avin- | proline | ARA_LEAF | p-PcUBI | GC | 50 | 132 |
| | targeted | DRAFT_5467 | | | | | | |
| 33085 | non- | Avin- | proline | ARA_LEAF | p-PcUBI | GC | 51 | 152 |
| | targeted | DRAFT_5644 | | | | | | |
| 33457 | non- | Avin- | arginine | ARA_LEAF | p-PcUBI | LC | 62 | 368 |
| | targeted | DRAFT_5651 | | | | | | |
| 33457 | non- | Avin- | glutamine | ARA_LEAF | p-PcUBI | GC | 92 | 226 |
| | targeted | DRAFT_5651 | | | | | | |
| 33596 | non- | Avin- | proline | ARA_LEAF | p-PcUBI | LC | 42 | 112 |
| | targeted | DRAFT_6093 | | | | | | |
| 34044 | non- | Avin- | arginine | ARA_LEAF | p-PcUBI | LC | 99 | 186 |
| | targeted | DRAFT_6700 | | | | | | |
| 34044 | non- | Avin- | glutamine | ARA_LEAF | p-PcUBI | GC | 45 | 168 |
| | targeted | DRAFT_6700 | | | | | | |
| 34044 | non- | Avin- | proline | ARA_LEAF | p-PcUBI | LC | 47 | 112 |
| | targeted | DRAFT_6700 | | | | | | |
| 34204 | non- | Avin- | proline | ARA_LEAF | p-PcUBI | LC | 47 | 75 |
| | targeted | DRAFT_6864 | | | | | | |
| 34301 | non- | AX653549 | glutamate | ARA_LEAF | p-PcUBI | GC | 47 | 57 |
| | targeted | | | | | | | |
| 34301 | non- | AX653549 | glutamine | ARA_LEAF | p-PcUBI | GC | 58 | 355 |
| | targeted | | | | | | | |
| 34602 | non- | AY087308 | glutamine | ARA_LEAF | p-PcUBI | GC | 53 | 114 |
| | targeted | | | | | | | |
| 34889 | plastidic | B0004 | arginine | ARA_LEAF | p-Super | LC | 56 | 2425 |
| 34889 | plastidic | B0004 | glutamine | ARA_LEAF | p-Super | GC | 49 | 4802 |
| 34889 | plastidic | B0004 | proline | ARA_LEAF | p-Super | GC | 40 | 1754 |
| 35204 | non- | B0061 | proline | ARA_SEED_2 | p-USP | LC | 137 | 1181 |
| | targeted | | | | | | | |
| 35366 | plastidic | B0115 | glutamine | ARA_SEED_2 | p-USP | LC | 67 | 86 |
| 35482 | non- | B0124 | arginine | ARA_LEAF | p-Super | LC | 53 | 395 |
| | targeted | | | | | | | |
| 35482 | plastidic | B0124 | proline | ARA_SEED_2 | p-USP | GC | 47 | 389 |
| 35482 | non- | B0124 | proline | ARA_LEAF | p-Super | LC | 47 | 124 |
| | targeted | | | | | | | |
| 7081 | non- | B0161 | proline | ARA_LEAF | p-Super | GC | 100 | 233 |
| | targeted | | | | | | | |
| 35590 | non- | B0221 | arginine | ARA_LEAF | p-Super | LC | 61 | 202 |
| | targeted | | | | | | | |
| 35590 | non- | B0221 | glutamate | ARA_LEAF | p-Super | GC | 54 | 78 |
| | targeted | | | | | | | |
| 35733 | plastidic | B0344 | proline | ARA_SEED_2 | p-USP | LC | 118 | 227 |
| 7333 | non- | B0449 | glutamine | ARA_LEAF | p-Super | GC | 51 | 961 |
| | targeted | | | | | | | |
| 35875 | non- | B0456 | proline | ARA_LEAF | p-Super | GC | 75 | 121 |
| | targeted | | | | | | | |
| 7686 | non- | B0486 | arginine | ARA_LEAF | p-Super | LC | 67 | 213 |
| | targeted | | | | | | | |
| 7686 | non- | B0486 | glutamate | ARA_LEAF | p-Super | LC | 33 | 57 |
| | targeted | | | | | | | |
| 35936 | non- | B0518 | glutamine | ARA_LEAF | p-Super | GC | 48 | 190 |
| | targeted | | | | | | | |
| 35967 | plastidic | B0593 | arginine | ARA_LEAF | p-Super | LC | 137 | 775 |
| 35967 | plastidic | B0593 | glutamate | ARA_LEAF | p-Super | LC | 36 | 80 |
| 35967 | plastidic | B0593 | glutamine | ARA_LEAF | p-Super | LC | 34 | 132 |
| 35967 | plastidic | B0593 | proline | ARA_LEAF | p-Super | LC | 71 | 148 |
| 36114 | non- | B0752 | glutamine | ARA_LEAF | p-Super | GC | 60 | 126 |
| | targeted | | | | | | | |
| 36299 | non- | B0828 | glutamine | ARA_LEAF | p-Super | LC | 35 | 80 |
| | targeted | | | | | | | |
| 36489 | non- | B0885 | glutamine | ARA_LEAF | p-Super | LC | 29 | 80 |
| | targeted | | | | | | | |
| 36489 | non- | B0885 | proline | ARA_LEAF | p-Super | GC | 50 | 181 |
| | targeted | | | | | | | |
| 7917 | non- | B0898 | glutamate | ARA_LEAF | p-Super | GC | 39 | 138 |
| | targeted | | | | | | | |
| 7917 | non- | B0898 | proline | ARA_LEAF | p-Super | GC | 68 | 8167 |
| | targeted | | | | | | | |
| 36623 | non- | B0962 | proline | ARA_SEED_2 | p-USP | LC | 114 | 475 |
| | targeted | | | | | | | |
| 36670 | non- | B0963 | proline | ARA_LEAF | p-Super | GC | 40 | 332 |
| | targeted | | | | | | | |
| 36809 | non- | B0980 | proline | ARA_SEED_2 | p-USP | LC | 233 | 304 |
| | targeted | | | | | | | |
| 7941 | non- | B1003 | glutamine | ARA_LEAF | p-Super | GC | 71 | 2938 |
| | targeted | | | | | | | |
| 7941 | non- | B1003 | proline | ARA_LEAF | p-Super | GC | 56 | 1315 |
| | targeted | | | | | | | |
| 36880 | non- | B1023 | arginine | ARA_LEAF | p-Super | LC | 53 | 554 |
| | targeted | | | | | | | |
| 36907 | non- | B1024 | glutamate | ARA_LEAF | p-Super | GC | 38 | 55 |
| | targeted | | | | | | | |
| 36937 | non- | B1108 | glutamine | ARA_LEAF | p-Super | GC | 61 | 491 |
| | targeted | | | | | | | |
| 36937 | non- | B1108 | proline | ARA_LEAF | p-Super | GC | 34 | 225 |
| | targeted | | | | | | | |
| 36971 | plastidic | B1136 | proline | ARA_LEAF | p-Super | GC | 47 | 549 |
| 37390 | non- | B1137 | arginine | ARA_LEAF | p-Super | LC | 85 | 748 |
| | targeted | | | | | | | |
| 37390 | non- | B1137 | glutamate | ARA_LEAF | p-Super | GC | 48 | 69 |
| | targeted | | | | | | | |
| 37390 | non- | B1137 | proline | ARA_LEAF | p-Super | GC | 40 | 169 |
| | targeted | | | | | | | |
| 37394 | non- | B1163 | glutamine | ARA_LEAF | p-Super | LC | 30 | 36 |
| | targeted | | | | | | | |
| 37400 | non- | B1186 | proline | ARA_LEAF | p-Super | GC | 36 | 164 |
| | targeted | | | | | | | |
| 37483 | non- | B1255 | glutamine | ARA_LEAF | p-Super | GC | 42 | 80 |
| | targeted | | | | | | | |
| 37483 | non- | B1255 | proline | ARA_LEAF | p-Super | GC | 39 | 520 |
| | targeted | | | | | | | |
| 37503 | non- | B1259 | arginine | ARA_LEAF | p-Super | LC | 66 | 105 |
| | targeted | | | | | | | |
| 37503 | non- | B1259 | glutamine | ARA_LEAF | p-Super | GC | 58 | 291 |
| | targeted | | | | | | | |
| 37539 | plastidic | B1263 | glutamine | ARA_SEED_2 | p-USP | LC | 68 | 176 |
| 37573 | non- | B1280 | glutamine | ARA_LEAF | p-Super | GC | 94 | 179 |
| | targeted | | | | | | | |
| 37658 | plastidic | B1297 | glutamine | ARA_SEED_2 | p-USP | LC | 70 | 412 |
| 37807 | non- | B1300 | glutamate | ARA_LEAF | p-Super | LC | 39 | 89 |
| | targeted | | | | | | | |
| 38226 | non- | B1330 | glutamate | ARA_LEAF | p-Super | LC | 50 | 97 |
| | targeted | | | | | | | |
| 38226 | non- | B1330 | glutamine | ARA_LEAF | p-Super | LC | 28 | 102 |
| | targeted | | | | | | | |
| 38266 | plastidic | B1431 | glutamate | ARA_LEAF | p-Super | LC | 33 | 52 |
| 38266 | plastidic | B1431 | glutamine | ARA_LEAF | p-Super | GC | 63 | 83 |
| 38289 | non- | B1445 | glutamate | ARA_LEAF | p-Super | LC | 36 | 165 |
| | targeted | | | | | | | |
| 38289 | non- | B1445 | glutamine | ARA_LEAF | p-Super | GC | 43 | 134 |
| | targeted | | | | | | | |
| 7947 | non- | B1522 | glutamate | ARA_LEAF | p-Super | GC | 38 | 281 |
| | targeted | | | | | | | |
| 38300 | non- | B1597 | glutamine | ARA_LEAF | p-Super | LC | 38 | 69 |
| | targeted | | | | | | | |
| 38345 | non- | B1627 | glutamine | ARA_LEAF | p-Super | GC | 43 | 239 |
| | targeted | | | | | | | |
| 38573 | non- | B1845 | glutamate | ARA_LEAF | p-Super | GC | 48 | 147 |
| | targeted | | | | | | | |
| 38767 | non- | B1898 | glutamate | ARA_LEAF | p-Super | LC | 40 | 45 |
| | targeted | | | | | | | |
| 38899 | non- | B1981 | glutamine | ARA_LEAF | p-Super | GC | 87 | 481 |
| | targeted | | | | | | | |
| 38947 | non- | B2063 | glutamate | ARA_LEAF | p-Super | GC | 57 | 134 |
| | targeted | | | | | | | |
| 38947 | non- | B2063 | glutamine | ARA_LEAF | p-Super | GC | 42 | 448 |
| | targeted | | | | | | | |
| 38947 | non- | B2063 | proline | ARA_LEAF | p-Super | GC | 54 | 722 |
| | targeted | | | | | | | |
| 8937 | mitochondrial | B2066 | glutamine | ARA_LEAF | p-Super | GC | 58 | 476 |
| 39002 | non- | B2107 | glutamate | ARA_LEAF | p-Super | GC | 35 | 185 |
| | targeted | | | | | | | |
| 39013 | non- | B2121 | glutamine | ARA_LEAF | p-Super | GC | 70 | 156 |
| | targeted | | | | | | | |
| 39040 | non- | B2178 | glutamate | ARA_LEAF | p-Super | GC | 75 | 139 |
| | targeted | | | | | | | |
| 39040 | non- | B2178 | glutamine | ARA_LEAF | p-Super | GC | 43 | 350 |
| | targeted | | | | | | | |
| 39040 | non- | B2178 | proline | ARA_LEAF | p-Super | GC | 41 | 288 |
| | targeted | | | | | | | |
| 39120 | non- | B2281 | glutamine | ARA_SEED_2 | p-USP | LC | 76 | 275 |
| | targeted | | | | | | | |
| 39219 | non- | B2360 | glutamate | ARA_LEAF | p-Super | GC | 42 | 121 |
| | targeted | | | | | | | |
| 39237 | non- | B2399 | proline | ARA_LEAF | p-Super | GC | 146 | 3262 |
| | targeted | | | | | | | |
| 39255 | non- | B2405 | glutamate | ARA_LEAF | p-Super | LC | 33 | 66 |
| | targeted | | | | | | | |
| 39300 | non- | B2414 | arginine | ARA_LEAF | p-Super | LC | 49 | 161 |
| | targeted | | | | | | | |
| 39300 | non- | B2414 | glutamate | ARA_LEAF | p-Super | LC | 35 | 55 |
| | targeted | | | | | | | |
| 40299 | non- | B2461 | proline | ARA_LEAF | p-Super | GC | 40 | 408 |
| | targeted | | | | | | | |
| 40329 | non- | B2474 | proline | ARA_LEAF | p-Super | GC | 42 | 89 |
| | targeted | | | | | | | |
| 9167 | non- | B2513 | glutamate | ARA_LEAF | p-Super | GC | 39 | 228 |
| | targeted | | | | | | | |
| 9167 | non- | B2513 | glutamine | ARA_LEAF | p-Super | GC | 63 | 239 |
| | targeted | | | | | | | |
| 40383 | non- | B2541 | arginine | ARA_LEAF | p-Super | LC | 121 | 441 |
| | targeted | | | | | | | |
| 40383 | non- | B2541 | glutamate | ARA_LEAF | p-Super | LC | 40 | 107 |
| | targeted | | | | | | | |
| 40637 | non- | B2548 | glutamine | ARA_LEAF | p-Super | LC | 36 | 62 |
| | targeted | | | | | | | |
| 40665 | non- | B2613 | glutamine | ARA_LEAF | p-Super | LC | 38 | 52 |
| | targeted | | | | | | | |
| 40665 | non- | B2613 | proline | ARA_LEAF | p-Super | GC | 33 | 191 |
| | targeted | | | | | | | |
| 40726 | non- | B2634 | glutamate | ARA_LEAF | p-Super | GC | 56 | 220 |
| | targeted | | | | | | | |
| 40726 | non- | B2634 | glutamine | ARA_LEAF | p-Super | GC | 92 | 1167 |
| | targeted | | | | | | | |
| 40726 | non- | B2634 | proline | ARA_LEAF | p-Super | GC | 54 | 1631 |
| | targeted | | | | | | | |
| 9244 | non- | B2673 | glutamate | ARA_LEAF | p-Super | GC | 102 | 157 |
| | targeted | | | | | | | |
| 9244 | non- | B2673 | glutamine | ARA_LEAF | p-Super | GC | 126 | 263 |
| | targeted | | | | | | | |
| 40741 | non- | B2714 | glutamine | ARA_LEAF | p-Super | LC | 37 | 46 |
| | targeted | | | | | | | |
| 40741 | non- | B2714 | proline | ARA_LEAF | p-Super | LC | 50 | 67 |
| | targeted | | | | | | | |
| 40795 | non- | B2812 | glutamate | ARA_LEAF | p-Super | GC | 37 | 130 |
| | targeted | | | | | | | |
| 40795 | non- | B2812 | glutamine | ARA_LEAF | p-Super | LC | 32 | 50 |
| | targeted | | | | | | | |
| 40795 | non- | B2812 | proline | ARA_LEAF | p-Super | LC | 43 | 104 |
| | targeted | | | | | | | |
| 40984 | non- | B2846 | proline | ARA_LEAF | p-Super | LC | 50 | 63 |
| | targeted | | | | | | | |
| 41006 | non- | B2909 | arginine | ARA_LEAF | p-Super | LC | 69 | 445 |
| | targeted | | | | | | | |
| 41006 | non- | B2909 | proline | ARA_LEAF | p-Super | GC | 39 | 248 |
| | targeted | | | | | | | |
| 41073 | non- | B2914 | glutamate | ARA_SEED_2 | p-USP | GC | 44 | 95 |
| | targeted | | | | | | | |
| 9333 | non- | B2923 | glutamate | ARA_LEAF | p-Super | LC | 41 | 77 |
| | targeted | | | | | | | |
| 41442 | non- | B2936 | proline | ARA_LEAF | p-Super | GC | 50 | 178 |
| | targeted | | | | | | | |
| 41499 | non- | B2957 | proline | ARA_LEAF | p-Super | GC | 34 | 262 |
| | targeted | | | | | | | |
| 41732 | plastidic | B2963 | proline | ARA_LEAF | p-Super | LC | 53 | 57 |
| 41797 | non- | B2999 | arginine | ARA_LEAF | p-Super | LC | 52 | 278 |
| | targeted | | | | | | | |
| 42046 | non- | B3064 | proline | ARA_SEED_2 | p-Super | GC | 57 | 255 |
| | targeted | | | | | | | |
| 42471 | non- | B3121 | arginine | ARA_LEAF | p-Super | LC | 63 | 201 |
| | targeted | | | | | | | |
| 42471 | non- | B3121 | proline | ARA_LEAF | p-Super | GC | 33 | 104 |
| | targeted | | | | | | | |
| 42477 | non- | B3151 | proline | ARA_LEAF | p-Super | GC | 45 | 689 |
| | targeted | | | | | | | |
| 9492 | non- | B3256 | arginine | ARA_SEED_2 | p-USP | LC | 100 | 2020 |
| | targeted | | | | | | | |
| 9492 | non- | B3256 | glutamate | ARA_SEED_2 | p-USP | GC | 57 | 194 |
| | targeted | | | | | | | |
| 42502 | non- | B3262 | arginine | ARA_LEAF | p-Super | LC | 50 | 273 |
| | targeted | | | | | | | |
| 42502 | non- | B3262 | glutamate | ARA_LEAF | p-Super | GC | 79 | 126 |
| | targeted | | | | | | | |
| 42502 | non- | B3262 | glutamine | ARA_LEAF | p-Super | LC | 27 | 113 |
| | targeted | | | | | | | |
| 42502 | non- | B3262 | proline | ARA_LEAF | p-Super | LC | 78 | 302 |
| | targeted | | | | | | | |
| 10104 | non- | B3346 | proline | ARA_LEAF | p-Super | LC | 48 | 193 |
| | targeted | | | | | | | |
| 42559 | non- | B3410 | glutamine | ARA_LEAF | p-Super | LC | 40 | 86 |
| | targeted | | | | | | | |
| 42579 | non- | B3427 | arginine | ARA_LEAF | p-Super | LC | 66 | 148 |
| | targeted | | | | | | | |
| 42592 | non- | B3509 | glutamate | ARA_LEAF | p-Super | LC | 47 | 65 |
| | targeted | | | | | | | |
| 10172 | plastidic | B3572 | proline | ARA_SEED_2 | p-USP | GC | 55 | 376 |
| 42600 | non- | B3616 | glutamine | ARA_LEAF | p-Super | GC | 45 | 225 |
| | targeted | | | | | | | |
| 42600 | non- | B3616 | proline | ARA_LEAF | p-Super | LC | 49 | 107 |
| | targeted | | | | | | | |
| 42931 | non- | B3634 | proline | ARA_LEAF | p-Super | GC | 34 | 162 |
| | targeted | | | | | | | |
| 43248 | non- | B3732 | glutamine | ARA_LEAF | p-Super | LC | 30 | 36 |
| | targeted | | | | | | | |
| 43800 | non- | B3793 | arginine | ARA_LEAF | p-Super | LC | 58 | 229 |
| | targeted | | | | | | | |
| 43800 | non- | B3793 | glutamate | ARA_LEAF | p-Super | GC | 78 | 212 |
| | targeted | | | | | | | |
| 43839 | non- | B3813 | proline | ARA_SEED_2 | p-USP | LC | 104 | 159 |
| | targeted | | | | | | | |
| 44196 | non- | B3814 | glutamine | ARA_LEAF | p-Super | GC | 54 | 94 |
| | targeted | | | | | | | |
| 10708 | non- | B3817 | proline | ARA_LEAF | p-Super | GC | 607 | 890 |
| | targeted | | | | | | | |
| 44223 | non- | B3945 | glutamine | ARA_SEED_2 | p-USP | LC | 68 | 125 |
| | targeted | | | | | | | |
| 44372 | non- | B3989 | glutamine | ARA_LEAF | p-Super | LC | 28 | 90 |
| | targeted | | | | | | | |
| 44378 | non- | B4012 | glutamate | ARA_SEED_2 | p-USP | GC | 39 | 76 |
| | targeted | | | | | | | |
| 10740 | non- | B4029 | arginine | ARA_LEAF | p-Super | LC | 58 | 478 |
| | targeted | | | | | | | |
| 10740 | non- | B4029 | glutamine | ARA_LEAF | p-Super | GC | 52 | 2052 |
| | targeted | | | | | | | |
| 10740 | non- | B4029 | proline | ARA_LEAF | p-Super | GC | 32 | 836 |
| | targeted | | | | | | | |
| 44466 | non- | B4067 | arginine | ARA_LEAF | p-Super | LC | 46 | 154 |
| | targeted | | | | | | | |
| 44609 | non- | B4121 | arginine | ARA_LEAF | p-Super | LC | 46 | 442 |
| | targeted | | | | | | | |
| 44609 | non- | B4121 | proline | ARA_LEAF | p-Super | GC | 33 | 151 |
| | targeted | | | | | | | |
| 44662 | non- | B4129 | proline | ARA_SEED_2 | p-USP | LC | 126 | 168 |
| | targeted | | | | | | | |
| 45022 | non- | B4139 | glutamine | ARA_SEED_2 | p-USP | LC | 196 | 629 |
| | targeted | | | | | | | |
| 45321 | non- | B4256 | arginine | ARA_LEAF | p-Super | LC | 93 | 838 |
| | targeted | | | | | | | |
| 45321 | non- | B4256 | glutamine | ARA_LEAF | p-Super | LC | 43 | 153 |
| | targeted | | | | | | | |
| 45321 | non- | B4256 | proline | ARA_LEAF | p-Super | GC | 39 | 843 |
| | targeted | | | | | | | |
| 45394 | non- | B4321 | arginine | ARA_LEAF | p-Super | LC | 75 | 181 |
| | targeted | | | | | | | |
| 45556 | non- | B4384 | glutamate | ARA_SEED_2 | p-USP | GC | 59 | 124 |
| | targeted | | | | | | | |
| 45556 | non- | B4384 | proline | ARA_SEED_2 | p-USP | LC | 107 | 506 |
| | targeted | | | | | | | |
| 45757 | non- | C_pp004096192r | arginine | ARA_LEAF | p-Super | LC | 61 | 306 |
| | targeted | | | | | | | |
| 45795 | non- | GM02LC11114 | proline | ARA_LEAF | p-PcUBI | GC | 40 | 127 |
| | targeted | | | | | | | |
| 10811 | non- | GM02LC12622 | glutamine | ARA_LEAF | p-PcUBI | GC | 68 | 261 |
| | targeted | | | | | | | |
| 45897 | non- | GM02LC15313 | arginine | ARA_LEAF | p-PcUBI | LC | 63 | 488 |
| | targeted | | | | | | | |
| 45897 | non- | GM02LC15313 | proline | ARA_LEAF | p-PcUBI | GC | 37 | 144 |
| | targeted | | | | | | | |
| 46405 | non- | GM02LC17485 | glutamine | ARA_LEAF | p-PcUBI | GC | 45 | 157 |
| | targeted | | | | | | | |
| 46515 | non- | GM02LC17556 | arginine | ARA_LEAF | p-PcUBI | LC | 98 | 362 |
| | targeted | | | | | | | |
| 46751 | non- | GM02LC19289 | proline | ARA_LEAF | p-PcUBI | GC | 36 | 231 |
| | targeted | | | | | | | |
| 46850 | non- | GM02LC44512 | proline | ARA_LEAF | p-PcUBI | GC | 72 | 410 |
| | targeted | | | | | | | |
| 47026 | non- | GM02LC46 | proline | ARA_LEAF | p-PcUBI | LC | 59 | 113 |
| | targeted | | | | | | | |
| 47076 | non- | GM02LC5744 | proline | ARA_LEAF | p-PcUBI | LC | 42 | 73 |
| | targeted | | | | | | | |
| 47105 | non- | GM02LC6021 | glutamate | ARA_LEAF | p-PcUBI | GC | 37 | 66 |
| | targeted | | | | | | | |
| 47159 | non- | NZ_AAAU02000016.150 | proline | ARA_LEAF | p-PcUBI | GC | 37 | 58 |
| | targeted | | | | | | | |
| 59851 | non- | S_pp015018333r | arginine | ARA_LEAF | p-Super | LC | 106 | 460 |
| | targeted | | | | | | | |
| 59851 | non- | S_pp015018333r | glutamine | ARA_LEAF | p-Super | GC | 44 | 187 |
| | targeted | | | | | | | |
| 47526 | mitochondrial | Sll0064 | proline | ARA_LEAF | p-PcUBI | GC | 62 | 111 |
| 47566 | plastidic | Sll0080 | glutamate | ARA_LEAF | p-PcUBI | LC | 51 | 61 |
| 48138 | non- | Sll0170 | glutamate | ARA_LEAF | p-PcUBI | GC | 39 | 234 |
| | targeted | | | | | | | |
| 49143 | plastidic | Sll0248 | arginine | ARA_LEAF | p-PcUBI | LC | 49 | 438 |
| 49342 | non- | Sll0250 | proline | ARA_LEAF | p-PcUBI | GC | 139 | 160 |
| | targeted | | | | | | | |
| 49800 | mitochondrial | Sll0254 | glutamine | ARA_LEAF | p-PcUBI | GC | 49 | 391 |
| 49800 | mitochondrial | Sll0254 | proline | ARA_LEAF | p-PcUBI | GC | 40 | 179 |
| 49828 | non- | Sll0290 | proline | ARA_LEAF | p-PcUBI | GC | 34 | 190 |
| | targeted | | | | | | | |
| 50070 | non- | Sll0354 | proline | ARA_LEAF | p-PcUBI | GC | 42 | 129 |
| | targeted | | | | | | | |
| 50104 | non- | Sll0420 | proline | ARA_LEAF | p-PcUBI | GC | 33 | 45 |
| | targeted | | | | | | | |
| 50339 | plastidic | Sll0521 | arginine | ARA_LEAF | p-PcUBI | LC | 51 | 185 |
| 50713 | mitochondrial | Sll0622 | proline | ARA_LEAF | p-PcUBI | LC | 46 | 106 |
| 50950 | non- | Sll0682 | glutamate | ARA_LEAF | p-PcUBI | GC | 45 | 153 |
| | targeted | | | | | | | |
| 50950 | non- | Sll0682 | proline | ARA_LEAF | p-PcUBI | GC | 32 | 90 |
| | targeted | | | | | | | |
| 51198 | non- | Sll0816 | arginine | ARA_LEAF | p-PcUBI | LC | 61 | 392 |
| | targeted | | | | | | | |
| 51198 | non- | Sll0816 | glutamate | ARA_LEAF | p-PcUBI | LC | 39 | 190 |
| | targeted | | | | | | | |
| 51198 | non- | Sll0816 | glutamine | ARA_LEAF | p-PcUBI | GC | 81 | 382 |
| | targeted | | | | | | | |
| 51268 | plastidic | Sll0891 | arginine | ARA_LEAF | p-PcUBI | LC | 47 | 289 |
| 51268 | plastidic | Sll0891 | glutamine | ARA_LEAF | p-PcUBI | LC | 28 | 28 |
| 51268 | plastidic | Sll0891 | proline | ARA_LEAF | p-PcUBI | LC | 52 | 77 |
| 51632 | mitochondrial | Sll0901 | proline | ARA_LEAF | p-PcUBI | GC | 38 | 273 |
| 52246 | mitochondrial | Sll0934 | glutamine | ARA_LEAF | p-PcUBI | GC | 72 | 148 |
| 52364 | non- | Sll0945 | proline | ARA_LEAF | p-PcUBI | GC | 34 | 293 |
| | targeted | | | | | | | |
| 52634 | mitochondrial | Sll1031 | glutamate | ARA_LEAF | p-PcUBI | LC | 37 | 133 |
| 52634 | mitochondrial | Sll1031 | glutamine | ARA_LEAF | p-PcUBI | GC | 55 | 461 |
| 52634 | mitochondrial | Sll1031 | proline | ARA_LEAF | p-PcUBI | GC | 32 | 110 |
| 52660 | plastidic | Sll1056 | proline | ARA_LEAF | p-PcUBI | LC | 45 | 113 |
| 53189 | non- | Sll1185 | glutamate | ARA_LEAF | p-PcUBI | LC | 33 | 55 |
| | targeted | | | | | | | |
| 53456 | non- | Sll1393 | glutamine | ARA_LEAF | p-PcUBI | GC | 49 | 73 |
| | targeted | | | | | | | |
| 53456 | non- | Sll1393 | proline | ARA_LEAF | p-PcUBI | GC | 36 | 105 |
| | targeted | | | | | | | |
| 53608 | non- | Sll1441 | glutamine | ARA_LEAF | p-PcUBI | LC | 37 | 37 |
| | targeted | | | | | | | |
| 53878 | mitochondrial | Sll1443 | glutamate | ARA_LEAF | p-PcUBI | LC | 33 | 61 |
| 54337 | mitochondrial | Sll1450 | glutamate | ARA_LEAF | p-PcUBI | LC | 34 | 90 |
| 54452 | plastidic | Sll1522 | glutamate | ARA_LEAF | p-PcUBI | GC | 63 | 154 |
| 11423 | non- | Sll1545 | arginine | ARA_LEAF | p-PcUBI | LC | 51 | 152 |
| | targeted | | | | | | | |
| 11423 | mitochondrial | Sll1545 | glutamine | ARA_LEAF | p-PcUBI | LC | 29 | 35 |
| 11423 | mitochondrial | Sll1545 | proline | ARA_LEAF | p-PcUBI | GC | 75 | 130 |
| 54804 | non- | Sll1546 | arginine | ARA_LEAF | p-PcUBI | LC | 64 | 260 |
| | targeted | | | | | | | |
| 54897 | non- | Sll1676 | proline | ARA_LEAF | p-PcUBI | LC | 44 | 186 |
| | targeted | | | | | | | |
| 55063 | plastidic | Sll1682 | proline | ARA_LEAF | p-PcUBI | GC | 76 | 272 |
| 55379 | non- | Sll1761 | arginine | ARA_LEAF | p-PcUBI | LC | 69 | 387 |
| | targeted | | | | | | | |
| 55379 | non- | Sll1761 | proline | ARA_LEAF | p-PcUBI | GC | 36 | 269 |
| | targeted | | | | | | | |
| 11471 | mitochondrial | Sll1917 | glutamate | ARA_LEAF | p-PcUBI | GC | 42 | 167 |
| 11471 | mitochondrial | Sll1917 | glutamine | ARA_LEAF | p-PcUBI | GC | 47 | 318 |
| 11471 | mitochondrial | Sll1917 | proline | ARA_LEAF | p-PcUBI | GC | 34 | 176 |
| 55385 | mitochondrial | Sll1920 | glutamate | ARA_LEAF | p-PcUBI | GC | 46 | 113 |
| 55385 | mitochondrial | Sll1920 | glutamine | ARA_LEAF | p-PcUBI | GC | 44 | 372 |
| 55771 | non- | Slr0237 | arginine | ARA_LEAF | p-PcUBI | LC | 84 | 104 |
| | targeted | | | | | | | |
| 55771 | non- | Slr0237 | glutamine | ARA_LEAF | p-PcUBI | LC | 30 | 40 |
| | targeted | | | | | | | |
| 55978 | plastidic | Slr0477 | proline | ARA_LEAF | p-PcUBI | LC | 42 | 86 |
| 56153 | plastidic | Slr0597 | glutamate | ARA_LEAF | p-PcUBI | GC | 43 | 100 |
| 56514 | non- | Slr0600 | proline | ARA_LEAF | p-PcUBI | LC | 43 | 60 |
| | targeted | | | | | | | |
| 56576 | mitochondrial | Slr0661 | proline | ARA_LEAF | p-PcUBI | LC | 45 | 85 |
| 56894 | mitochondrial | Slr0710 | arginine | ARA_LEAF | p-PcUBI | LC | 47 | 175 |
| 57235 | plastidic | Slr0739 | arginine | ARA_LEAF | p-PcUBI | LC | 347 | 910 |
| 57235 | plastidic | Slr0739 | glutamine | ARA_LEAF | p-PcUBI | GC | 46 | 519 |
| 57235 | plastidic | Slr0739 | proline | ARA_LEAF | p-PcUBI | GC | 105 | 801 |
| 57663 | non- | Slr0756 | arginine | ARA_LEAF | p-PcUBI | LC | 48 | 280 |
| | targeted | | | | | | | |
| 57663 | non- | Slr0756 | proline | ARA_LEAF | p-PcUBI | GC | 32 | 91 |
| | targeted | | | | | | | |
| 57679 | mitochondrial | Slr0782 | proline | ARA_LEAF | p-PcUBI | LC | 43 | 94 |
| 57734 | non- | Slr1096 | glutamate | ARA_LEAF | p-PcUBI | LC | 41 | 79 |
| | targeted | | | | | | | |
| 58058 | non- | Slr1269 | arginine | ARA_LEAF | p-PcUBI | LC | 65 | 209 |
| | targeted | | | | | | | |
| 58058 | non- | Slr1269 | glutamate | ARA_LEAF | p-PcUBI | GC | 63 | 150 |
| | targeted | | | | | | | |
| 58324 | non- | Slr1312 | arginine | ARA_LEAF | p-PcUBI | LC | 52 | 306 |
| | targeted | | | | | | | |
| 58472 | mitochondrial | Slr1369 | glutamate | ARA_LEAF | p-PcUBI | GC | 39 | 86 |
| 58590 | non- | Slr1420 | glutamate | ARA_LEAF | p-PcUBI | LC | 35 | 196 |
| | targeted | | | | | | | |
| 58668 | mitochondrial | Slr1492 | glutamine | ARA_LEAF | p-PcUBI | LC | 30 | 46 |
| 12070 | mitochondrial | Slr1655 | proline | ARA_LEAF | p-PcUBI | GC | 40 | 164 |
| 58731 | plastidic | Slr1739 | arginine | ARA_LEAF | p-PcUBI | LC | 99 | 193 |
| 58731 | plastidic | Slr1739 | glutamine | ARA_LEAF | p-PcUBI | GC | 42 | 229 |
| 58751 | mitochondrial | Slr1742 | proline | ARA_LEAF | p-PcUBI | LC | 49 | 75 |
| 58823 | mitochondrial | Slr1755 | glutamate | ARA_LEAF | p-PcUBI | GC | 40 | 62 |
| 12140 | plastidic | Slr1791 | proline | ARA_LEAF | p-PcUBI | GC | 32 | 53 |
| 59041 | non- | Slr1882 | glutamate | ARA_LEAF | p-PcUBI | LC | 34 | 114 |
| | targeted | | | | | | | |
| 59165 | plastidic | Slr2023 | glutamate | ARA_LEAF | p-PcUBI | GC | 123 | 185 |
| 59165 | plastidic | Slr2023 | glutamine | ARA_LEAF | p-PcUBI | GC | 70 | 355 |
| 59370 | plastidic | Slr2124 | proline | ARA_LEAF | p-PcUBI | LC | 53 | 228 |
| 12698 | non- | TTC0019 | glutamine | ARA_LEAF | p-PcUBI | LC | 72 | 162 |
| | targeted | | | | | | | |
| 12698 | non- | TTC0019 | proline | ARA_LEAF | p-PcUBI | GC | 45 | 228 |
| | targeted | | | | | | | |
| 60301 | non- | TTC0035 | proline | ARA_LEAF | p-PcUBI | LC | 44 | 145 |
| | targeted | | | | | | | |
| 60859 | non- | TTC0216 | proline | ARA_LEAF | p-PcUBI | GC | 37 | 137 |
| | targeted | | | | | | | |
| 61070 | non- | TTC0337 | proline | ARA_LEAF | p-PcUBI | GC | 37 | 74 |
| | targeted | | | | | | | |
| 61532 | non- | TTC0768 | proline | ARA_LEAF | p-PcUBI | LC | 48 | 77 |
| | targeted | | | | | | | |
| 61553 | non- | TTC0917 | glutamine | ARA_LEAF | p-PcUBI | GC | 58 | 79 |
| | targeted | | | | | | | |
| 61723 | non- | TTC1193 | glutamate | ARA_LEAF | p-PcUBI | LC | 35 | 73 |
| | targeted | | | | | | | |
| 62079 | non- | TTC1386 | glutamate | ARA_LEAF | p-PcUBI | GC | 66 | 106 |
| | targeted | | | | | | | |
| 12974 | non- | TTC1550 | glutamine | ARA_LEAF | p-PcUBI | GC | 56 | 248 |
| | targeted | | | | | | | |
| 12974 | non- | TTC1550 | proline | ARA_LEAF | p-PcUBI | LC | 54 | 320 |
| | targeted | | | | | | | |
| 62160 | non- | TTC1918 | glutamate | ARA_LEAF | p-PcUBI | GC | 43 | 128 |
| | targeted | | | | | | | |
| 62160 | non- | TTC1918 | glutamine | ARA_LEAF | p-PcUBI | GC | 42 | 139 |
| | targeted | | | | | | | |
| 62244 | non- | XM_473199 | glutamate | ARA_LEAF | p-PcUBI | GC | 45 | 75 |
| | targeted | | | | | | | |
| 62244 | non- | XM_473199 | glutamine | ARA_LEAF | p-PcUBI | GC | 62 | 173 |
| | targeted | | | | | | | |
| 62524 | non- | Ybl021c | proline | ARA_LEAF | Big35S | GC | 35 | 804 |
| | targeted | | | | | | | |
| 62717 | non- | Ybr160w | glutamate | ARA_LEAF | Big35S | GC | 37 | 118 |
| | targeted | | | | | | | |
| 62717 | non- | Ybr160w | proline | ARA_LEAF | Big35S | GC | 36 | 323 |
| | targeted | | | | | | | |
| 63167 | non- | Ycl026c-a | proline | ARA_LEAF | Big35S | LC | 51 | 68 |
| | targeted | | | | | | | |
| 63264 | plastidic | Ycr102c | arginine | ARA_LEAF | p-Super | LC | 78 | 107 |
| 14302 | plastidic | Ydl168w | proline | ARA_LEAF | p-Super | LC | 42 | 99 |
| 63334 | plastidic | Ydr044w | proline | ARA_LEAF | p-PcUBI | LC | 47 | 228 |
| 63544 | plastidic | Ydr046c | proline | ARA_LEAF | p-Super | LC | 66 | 75 |
| 63665 | plastidic | Ydr265w | arginine | ARA_LEAF | p-PcUBI | LC | 88 | 276 |
| 63713 | plastidic | Ydr338c | arginine | ARA_LEAF | p-Super | LC | 67 | 165 |
| 63745 | plastidic | Yer014w | glutamine | ARA_LEAF | p-PcUBI | GC | 74 | 186 |
| 63803 | plastidic | Yer106w | glutamine | ARA_LEAF | p-Super | GC | 43 | 118 |
| 63807 | non- | Yfl016c | proline | ARA_LEAF | Big35S | GC | 43 | 83 |
| | targeted | | | | | | | |
| 64144 | non- | Yfl019c | proline | ARA_LEAF | Big35S | GC | 32 | 235 |
| | targeted | | | | | | | |
| 64148 | plastidic | Yfl054c | arginine | ARA_LEAF | p-Super | LC | 87 | 268 |
| 64148 | plastidic | Yfl054c | glutamine | ARA_LEAF | p-Super | LC | 30 | 75 |
| 64157 | plastidic | Ygl174w | arginine | ARA_LEAF | p-Super | LC | 85 | 199 |
| 64177 | non- | Ygl237c | proline | ARA_LEAF | Big35S | LC | 42 | 388 |
| | targeted | | | | | | | |
| 64198 | plastidic | Ygr068c | glutamine | ARA_LEAF | p-Super | GC | 60 | 428 |
| 64218 | plastidic | Ygr175c | proline | ARA_LEAF | p-PcUBI | GC | 43 | 69 |
| 64315 | plastidic | Ygr221c | glutamate | ARA_LEAF | p-Super | LC | 33 | 65 |
| 64315 | plastidic | Ygr221c | glutamine | ARA_LEAF | p-Super | LC | 24 | 30 |
| 14715 | plastidic | Yhl013c | proline | ARA_LEAF | p-Super | GC | 33 | 402 |
| 64336 | plastidic | Yhr024c | arginine | ARA_LEAF | p-Super | LC | 72 | 166 |
| 64470 | non- | Yhr072w-a | proline | ARA_SEED_2 | p-USP | GC | 53 | 260 |
| | targeted | | | | | | | |
| 64546 | plastidic | Yhr207c | glutamate | ARA_LEAF | p-Super | LC | 33 | 47 |
| 64563 | plastidic | Yil074c | arginine | ARA_LEAF | p-Super | LC | 47 | 129 |
| 64773 | plastidic | Yir027c | proline | ARA_LEAF | p-Super | LC | 39 | 46 |
| 64894 | plastidic | Yir029w | arginine | ARA_LEAF | p-Super | LC | 52 | 316 |
| 64964 | plastidic | Yjl073w | glutamine | ARA_LEAF | p-Super | LC | 28 | 29 |
| 64975 | non- | Yjl088w | arginine | ARA_LEAF | Big35S | LC | 52 | 124 |
| | targeted | | | | | | | |
| 65181 | plastidic | Yjl129c | proline | ARA_LEAF | p-Super | GC | 37 | 89 |
| 14821 | plastidic | Yjl137c | arginine | ARA_LEAF | p-Super | LC | 47 | 152 |
| 65224 | non- | Yjl138c | proline | ARA_SEED_2 | p-PcUBI | LC | 112 | 770 |
| | targeted | | | | | | | |
| 66225 | plastidic | Yjr133w | glutamine | ARA_LEAF | p-Super | LC | 29 | 46 |
| 66274 | non- | Yjr153w | glutamate | ARA_LEAF | p-PcUBI | LC | 36 | 124 |
| | targeted | | | | | | | |
| 66419 | non- | Ykl106w | proline | ARA_LEAF | p-PcUBI | GC | 42 | 54 |
| | targeted | | | | | | | |
| 66695 | plastidic | Ylr065c | glutamate | ARA_LEAF | p-Super | LC | 39 | 62 |
| 66715 | plastidic | Ylr178c | glutamine | ARA_LEAF | p-Super | LC | 29 | 59 |
| 66772 | non- | Ylr304c | proline | ARA_LEAF | Big35S | LC | 42 | 53 |
| | targeted | | | | | | | |
| 67190 | plastidic | Ylr359w | proline | ARA_LEAF | p-Super | GC | 72 | 309 |
| 67299 | non- | Ynl142w | glutamine | ARA_SEED_2 | p-PcUBI | LC | 98 | 182 |
| | targeted | | | | | | | |
| 67646 | plastidic | Ynr019w | proline | ARA_LEAF | p-PcUBI | LC | 59 | 77 |
| 67684 | plastidic | Yol045w | arginine | ARA_LEAF | p-Super | LC | 49 | 140 |
| 67710 | non- | Yor168w | glutamate | ARA_LEAF | Big35S | LC | 38 | 112 |
| | targeted | | | | | | | |
| 67710 | non- | Yor168w | glutamine | ARA_LEAF | Big35S | LC | 28 | 112 |
| | targeted | | | | | | | |
| 67951 | non- | Yor221c | proline | ARA_SEED_2 | p-PcUBI | GC | 55 | 420 |
| | targeted | | | | | | | |
| 67968 | non- | Yor233w | proline | ARA_LEAF | Big35S | GC | 40 | 167 |
| | targeted | | | | | | | |
| 67998 | plastidic | Ypl117c | arginine | ARA_LEAF | p-PcUBI | LC | 77 | 341 |
| 68413 | non- | Zm_4842_BE510522 | arginine | ARA_LEAF | p-PcUBI | LC | 52 | 238 |
| | targeted | | | | | | | |
| 68413 | non- | Zm_4842_BE510522 | glutamine | ARA_LEAF | p-PcUBI | GC | 49 | 411 |
| | targeted | | | | | | | |
| 68413 | non- | Zm_4842_BE510522 | proline | ARA_LEAF | p-PcUBI | GC | 44 | 103 |
| | targeted | | | | | | | |
| 68132 | non- | ZM06LC1143 | arginine | ARA_LEAF | p-PcUBI | LC | 49 | 117 |
| | targeted | | | | | | | |
| 68363 | non- | ZM06LC11975 | proline | ARA_LEAF | p-PcUBI | GC | 33 | 136 |
| | targeted | | | | | | | |

Column 1 shows the SEQ ID NO, Column 2 shows the expression type (targeted or non-targeted), Column 3 shows the “gene name” (locus), Column 4 shows the metabolite analyzed, Column 5 indicates the A. thaliana source tissue analyzed, Column 6 indicates the used promoter for expression, Column 7 indicates the analytical method. Columns 8 and 9 show the minumum and the maximum increase of the analyzed metabolite (in percent) in comparison to the wild type (=ratio_by_weight, given as percent increase).

When the analyses were repeated independently, all results proved to be significant.

[0505.1.1.2] to [0515.1.1.2] for the disclosure of these paragraphs see [0505.1.1.1] to [0515.1.1.1] above.

In a further embodiment, the present invention relates in paragraphs

[0000.1.1.3] to [0514.1.1.3] to a further process for the production of at least a, preferably a, fine chemical selected from the group consisting of 3,4-dihydroxyphenylalanine (dopa), 5-oxoproline, alanine, asparagine, aspartate, citrulline, glycine, homoserine, ornithine, phenylalanine, serine, and/or tyrosine defined below and corresponding embodiments as described herein as follows [0001.1.1.3] to [0012.1.3.3] for the disclosure of these paragraphs see [0001.1.1.1] to [0011.1.1.1] above.

It is an object of the present invention to develop an inexpensive process for the synthesis of 3,4-dihydroxyphenylalanine (dopa), 5-oxoproline, alanine, asparagine, aspartate, citrulline, glycine, homoserine, ornithine, phenylalanine, serine, and/or tyrosine.

for the disclosure of this paragraph see [0013.1.1.1] above.

Accordingly, in a first embodiment, the invention relates to a process for the production of at least one, preferably a, fine chemical selected from the group consisting of: 3,4-dihydroxyphenylalanine (dopa), 5-oxoproline, alanine, asparagine, aspartate, citrulline, glycine, homoserine, ornithine, phenylalanine, serine and tyrosine, or, in other words, of the “fine chemical” or “fine chemical of the invention”.

The terms “fine chemical of the invention”, “fine chemical” or “the fine chemical” are used herein equally and relate in context of the paragraphs or sections [0014.1.3.3] to [0514.1.1.3] essentially to the metabolite or the metabolites indicated in column 7, application no. 3 of Tables I, II and IV in the respective line.

Further, the term “in context of any of the paragraphs [0014.1.3.3] to [0514.1.1.3]” as used herein means that for any of said paragraphs [0014.1.3.3] to [0514.1.1.3] the term “the fine chemical” is understood to follow the definition of paragraphs or sections [0014.1.3.3] and [0015.1.3.3], independently whether it refers to any other paragraph or not and whether the reference recites the term “fine chemical” in an other context.

Thus, in cases where one or more paragraphs or sections are incorporated by reference into any of the present paragraphs [0014.1.3.3] to [0514.1.1.3], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to the definition of paragraph [0014.1.3.3] and [0015.1.3.3].

Accordingly, in one embodiment, the term “fine chemical of the invention”, “fine chemical” or “the fine chemical” as used herein (alone or in combination, like in FCRP) means “3,4-dihydroxyphenylalanine (dopa) in context of the nucleic acid or polypeptide sequences listed in the respective same line of any one of Tables I to IV of application no. 3 and indicating in column 7 the metabolite “3,4-dihydroxyphenylalanine (dopa)”.

In one embodiment, the term 3,4-dihydroxyphenylalanine (dopa) or the term “fine chemical” mean in context of the paragraphs or sections [0014.1.3.3] to [0514.1.1.3] at least one chemical compound with an activity of the above mentioned 3,4-dihydroxyphenylalanine (dopa), respectively.

Accordingly, in one embodiment, the term “fine chemical of the invention”, “fine chemical” or “the fine chemical” as used herein (alone or in combination, like in FCRP) means “5-oxoproline in context of the nucleic acid or polypeptide sequences listed in the respective same line of any one of Tables Ito IV of application no. 3 and indicating in column 7 the metabolite “5-oxoproline”.

In one embodiment, the term 5-oxoproline or the term “fine chemical” mean in context of the paragraphs or sections [0014.1.3.3] to [0514.1.1.3] at least one chemical compound with an activity of the above mentioned 5-oxoproline, respectively.

Accordingly, in one embodiment, the term “fine chemical of the invention”, “fine chemical” or “the fine chemical” as used herein (alone or in combination, like in FCRP) means “alanine in context of the nucleic acid or polypeptide sequences listed in the respective same line of any one of Tables Ito IV of application no. 3 and indicating in column 7 the metabolite “alanine”.

In one embodiment, the term alanine or the term “fine chemical” mean in context of the paragraphs or sections [0014.1.3.3] to [0514.1.1.3] at least one chemical compound with an activity of the above mentioned alanine, respectively.

Accordingly, in one embodiment, the term “fine chemical of the invention”, “fine chemical” or “the fine chemical” as used herein (alone or in combination, like in FCRP) means “asparagine in context of the nucleic acid or polypeptide sequences listed in the respective same line of any one of Tables I to IV of application no. 3 and indicating in column 7 the metabolite “asparagine”.

In one embodiment, the term asparagine or the term “fine chemical” mean in context of the paragraphs or sections [0014.1.3.3] to [0514.1.1.3] at least one chemical compound with an activity of the above mentioned asparagine, respectively.

Accordingly, in one embodiment, the term “fine chemical of the invention”, “fine chemical” or “the fine chemical” as used herein (alone or in combination, like in FCRP) means “aspartate in context of the nucleic acid or polypeptide sequences listed in the respective same line of any one of Tables I to IV of application no. 3 and indicating in column 7 the metabolite “aspartate”.

In one embodiment, the term aspartate or the term “fine chemical” mean in context of the paragraphs or sections [0014.1.3.3] to [0514.1.1.3] at least one chemical compound with an activity of the above mentioned aspartate, respectively.

Accordingly, in one embodiment, the term “fine chemical of the invention”, “fine chemical” or “the fine chemical” as used herein (alone or in combination, like in FCRP) means “citrulline in context of the nucleic acid or polypeptide sequences listed in the respective same line of any one of Tables I to IV of application no. 3 and indicating in column 7 the metabolite “citrulline”.

In one embodiment, the term citrulline or the term “fine chemical” mean in context of the paragraphs or sections [0014.1.3.3] to [0514.1.1.3] at least one chemical compound with an activity of the above mentioned citrulline, respectively.

Accordingly, in one embodiment, the term “fine chemical of the invention”, “fine chemical” or “the fine chemical” as used herein (alone or in combination, like in FCRP) means “glycine in context of the nucleic acid or polypeptide sequences listed in the respective same line of any one of Tables I to IV of application no. 3 and indicating in column 7 the metabolite “glycine”.

In one embodiment, the term glycine or the term “fine chemical” mean in context of the paragraphs or sections [0014.1.3.3] to [0514.1.1.3] at least one chemical compound with an activity of the above mentioned glycine, respectively.

Accordingly, in one embodiment, the term “fine chemical of the invention”, “fine chemical” or “the fine chemical” as used herein (alone or in combination, like in FCRP) means “homoserine in context of the nucleic acid or polypeptide sequences listed in the respective same line of any one of Tables I to IV of application no. 3 and indicating in column 7 the metabolite “homoserine”.

In one embodiment, the term homoserine or the term “fine chemical” mean in context of the paragraphs or sections [0014.1.3.3] to [0514.1.1.3] at least one chemical compound with an activity of the above mentioned homoserine, respectively.

Accordingly, in one embodiment, the term “fine chemical of the invention”, “fine chemical” or “the fine chemical” as used herein (alone or in combination, like in FCRP) means “ornithine in context of the nucleic acid or polypeptide sequences listed in the respective same line of any one of Tables I to IV of application no. 3 and indicating in column 7 the metabolite “ornithine”.

In one embodiment, the term ornithine or the term “fine chemical” mean in context of the paragraphs or sections [0014.1.3.3] to [0514.1.1.3] at least one chemical compound with an activity of the above mentioned ornithine, respectively.

Accordingly, in one embodiment, the term “fine chemical of the invention”, “fine chemical” or “the fine chemical” as used herein (alone or in combination, like in FCRP) means “phenylalanine in context of the nucleic acid or polypeptide sequences listed in the respective same line of any one of Tables I to IV of application no. 3 and indicating in column 7 the metabolite “phenylalanine”.

In one embodiment, the term phenylalanine or the term “fine chemical” mean in context of the paragraphs or sections [0014.1.3.3] to [0514.1.1.3] at least one chemical compound with an activity of the above mentioned phenylalanine, respectively.

Accordingly, in one embodiment, the term “fine chemical of the invention”, “fine chemical” or “the fine chemical” as used herein (alone or in combination, like in FCRP) means “serine in context of the nucleic acid or polypeptide sequences listed in the respective same line of any one of

Tables I to IV of application no. 3 and indicating in column 7 the metabolite “serine”.

In one embodiment, the term serine or the term “fine chemical” mean in context of the paragraphs or sections [0014.1.3.3] to [0514.1.1.3] at least one chemical compound with an activity of the above mentioned serine, respectively.

Accordingly, in one embodiment, the term “fine chemical of the invention”, “fine chemical” or “the fine chemical” as used herein (alone or in combination, like in FCRP) means “tyrosine in context of the nucleic acid or polypeptide sequences listed in the respective same line of any one of Tables I to IV of application no. 3 and indicating in column 7 the metabolite “tyrosine”.

In one embodiment, the term tyrosine or the term “fine chemical” mean in context of the paragraphs or sections [0014.1.3.3] to [0514.1.1.3] at least one chemical compound with an activity of the above mentioned tyrosine, respectively.

Accordingly, in one embodiment, the terms “fine chemical of the invention”, “fine chemical” or “the fine chemical” mean in context of any of the paragraphs [0014.1.3.3] to [0514.1.1.3] 3,4-dihydroxyphenylalanine (dopa), preferably the L-enantiomer of 3,4-dihydroxyphenylalanine (dopa), its salts, ester or amides in free form or bound to proteins. In a preferred embodiment, the term “the fine chemical” means 3,4-dihydroxyphenylalanine (dopa) or its salts, in free form or bound to proteins. In a preferred embodiment, the term “the fine chemical” means the L-enantiomer of 3,4-dihydroxyphenylalanine (dopa).

On the other hand in case “3,4-dihydroxyphenylalanine (dopa)” is stated it means 3,4-dihydroxyphenylalanine (dopa) itself, its salts, ester or amides in free form or bound to proteins, preferably the L-enantiomer of 3,4-dihydroxyphenylalanine (dopa), its salts, ester or amides in free form or bound to proteins. In a preferred embodiment “3,4-dihydroxyphenylalanine (dopa)” means the L-enantiomer of 3,4-dihydroxyphenylalanine (dopa) in free form. In another preferred embodiment “3,4-dihydroxyphenylalanine (dopa)” means the L-enantiomer of 3,4-dihydroxyphenylalanine (dopa) bound to proteins.

Accordingly, in one embodiment, the terms “fine chemical of the invention”, “fine chemical” or “the fine chemical” mean in context of any of the paragraphs [0014.1.3.3] to [0514.1.1.3] 5oxoproline, preferably the L-enantiomer of 5-oxoproline, its salts, ester or amides in free form or bound to proteins. In a preferred embodiment, the term “the fine chemical” means 5-oxoproline or its salts, in free form or bound to proteins. In a preferred embodiment, the term “the fine chemical” means the L-enantiomer of 5-oxoproline.

On the other hand in case “5-oxoproline” is stated it means 5-oxoproline itself, its salts, ester or amides in free form or bound to proteins, preferably the L-enantiomer of 5-oxoproline, its salts, ester or amides in free form or bound to proteins. In a preferred embodiment “5-oxoproline” means the L-enantiomer of 5-oxoproline in free form. In another preferred embodiment “5-oxoproline” means the L-enantiomer of 5-oxoproline bound to proteins.

Accordingly, in one embodiment, the terms “fine chemical of the invention”, “fine chemical” or “the fine chemical” mean in context of any of the paragraphs [0014.1.3.3] to [0514.1.1.3] alanine, preferably the L-enantiomer of alanine, its salts, ester or amides in free form or bound to proteins. In a preferred embodiment, the term “the fine chemical” means alanine or its salts, in free form or bound to proteins. In a preferred embodiment, the term “the fine chemical” means the L-enantiomer of alanine.

On the other hand in case “alanine” is stated it means alanine itself, its salts, ester or amides in free form or bound to proteins, preferably the L-enantiomer of alanine, its salts, ester or amides in free form or bound to proteins. In a preferred embodiment “alanine” means the L-enantiomer of alanine in free form. In another preferred embodiment “alanine” means the L-enantiomer of alanine bound to proteins.

Accordingly, in one embodiment, the terms “fine chemical of the invention”, “fine chemical” or “the fine chemical” mean in context of any of the paragraphs [0014.1.3.3] to [0514.1.1.3] asparagine, preferably the L-enantiomer of asparagine, its salts, ester or amides in free form or bound to proteins. In a preferred embodiment, the term “the fine chemical” means asparagine or its salts, in free form or bound to proteins. In a preferred embodiment, the term “the fine chemical” means the L-enantiomer of asparagine.

On the other hand in case “asparagine” is stated it means asparagine itself, its salts, ester or amides in free form or bound to proteins, preferably the L-enantiomer of asparagine, its salts, ester or amides in free form or bound to proteins. In a preferred embodiment “asparagine” means the L-enantiomer of asparagine in free form. In another preferred embodiment “asparagine” means the L-enantiomer of asparagine bound to proteins.

Accordingly, in one embodiment, the terms “fine chemical of the invention”, “fine chemical” or “the fine chemical” mean in context of any of the paragraphs [0014.1.3.3] to [0514.1.1.3] aspartate, preferably the L-enantiomer of aspartate, its salts, ester or amides in free form or bound to proteins. In a preferred embodiment, the term “the fine chemical” means aspartate or its salts, in free form or bound to proteins. In a preferred embodiment, the term “the fine chemical” means the L-enantiomer of aspartate.

On the other hand in case “aspartate” is stated it means aspartate itself, its salts, ester or amides in free form or bound to proteins, preferably the L-enantiomer of aspartate, its salts, ester or amides in free form or bound to proteins. In a preferred embodiment “aspartate” means the L-enantiomer of aspartate in free form. In another preferred embodiment “aspartate” means the L-enantiomer of aspartate bound to proteins.

Accordingly, in one embodiment, the terms “fine chemical of the invention”, “fine chemical” or “the fine chemical” mean in context of any of the paragraphs [0014.1.3.3] to [0514 .1.1.3] citrulline, preferably the L-enantiomer of citrulline, its salts, ester or amides in free form or bound to proteins. In a preferred embodiment, the term “the fine chemical” means citrulline or its salts, in free form or bound to proteins. In a preferred embodiment, the term “the fine chemical” means the L-enantiomer of citrulline.

On the other hand in case “citrulline” is stated it means citrulline itself, its salts, ester or amides in free form or bound to proteins, preferably the L-enantiomer of citrulline, its salts, ester or amides in free form or bound to proteins. In a preferred embodiment “citrulline” means the L-enantiomer of citrulline in free form. In another preferred embodiment “citrulline” means the L-enantiomer of citrulline bound to proteins.

Accordingly, in one embodiment, the terms “fine chemical of the invention”, “fine chemical” or “the fine chemical” mean in context of any of the paragraphs [0014.1.3.3] to [0514.1.1.3] glycine, its salts, ester or amides in free form or bound to proteins. In a preferred embodiment, the term “the fine chemical” means glycine or its salts, in free form or bound to proteins.

On the other hand in case “glycine” is stated it means glycine itself, its salts, ester or amides in free form or bound to proteins

Accordingly, in one embodiment, the terms “fine chemical of the invention”, “fine chemical” or “the fine chemical” mean in context of any of the paragraphs [0014.1.3.3] to [0514.1.1.3] homoserine, preferably the L-enantiomer of homoserine, its salts, ester or amides in free form or bound to proteins. In a preferred embodiment, the term “the fine chemical” means homoserine or its salts, in free form or bound to proteins. In a preferred embodiment, the term “the fine chemical” means the L-enantiomer of homoserine.

On the other hand in case “homoserine” is stated it means homoserine itself, its salts, ester or amides in free form or bound to proteins, preferably the L-enantiomer of homoserine, its salts, ester or amides in free form or bound to proteins. In a preferred embodiment “homoserine” means the L-enantiomer of homoserine in free form. In another preferred embodiment “homoserine” means the L-enantiomer of homoserine bound to proteins.

Accordingly, in one embodiment, the terms “fine chemical of the invention”, “fine chemical” or “the fine chemical” mean in context of any of the paragraphs [0014.1.3.3] to [0514.1.1.3] ornithine, preferably the L-enantiomer of ornithine, its salts, ester or amides in free form or bound to proteins. In a preferred embodiment, the term “the fine chemical” means ornithine or its salts, in free form or bound to proteins. In a preferred embodiment, the term “the fine chemical” means the L-enantiomer of ornithine.

On the other hand in case “ornithine” is stated it means ornithine itself, its salts, ester or amides in free form or bound to proteins, preferably the L-enantiomer of ornithine, its salts, ester or amides in free form or bound to proteins. In a preferred embodiment “ornithine” means the L-enantiomer of ornithine in free form. In another preferred embodiment “ornithine” means the L-enantiomer of ornithine bound to proteins.

Accordingly, in one embodiment, the terms “fine chemical of the invention”, “fine chemical” or “the fine chemical” mean in context of any of the paragraphs [0014.1.3.3] to [0514.1.1.3] phenylalanine, preferably the L-enantiomer of phenylalanine, its salts, ester or amides in free form or bound to proteins. In a preferred embodiment, the term “the fine chemical” means phenylalanine or its salts, in free form or bound to proteins. In a preferred embodiment, the term “the fine chemical” means the L-enantiomer of phenylalanine. On the other hand in case “phenylalanine” is stated it means phenylalanine itself, its salts, ester or amides in free form or bound to proteins, preferably the L-enantiomer of phenylalanine, its salts, ester or amides in free form or bound to proteins. In a preferred embodiment “phenylalanine” means the L-enantiomer of phenylalanine in free form. In another preferred embodiment “phenylalanine” means the L-enantiomer of phenylalanine bound to proteins.

Accordingly, in one embodiment, the terms “fine chemical of the invention”, “fine chemical” or “the fine chemical” mean in context of any of the paragraphs [0014.1.3.3] to [0514.1.1.3] serine, preferably the L-enantiomer of serine, its salts, ester or amides in free form or bound to proteins. In a preferred embodiment, the term “the fine chemical” means serine or its salts, in free form or bound to proteins. In a preferred embodiment, the term “the fine chemical” means the L-enantiomer of serine.

On the other hand in case “serine” is stated it means serine itself, its salts, ester or amides in free form or bound to proteins, preferably the L-enantiomer of serine, its salts, ester or amides in free form or bound to proteins. In a preferred embodiment “serine” means the L-enantiomer of serine in free form. In another preferred embodiment “serine” means the L-enantiomer of serine bound to proteins.

Accordingly, in one embodiment, the terms “fine chemical of the invention”, “fine chemical” or “the fine chemical” mean in context of any of the paragraphs [0014.1.3.3] to [051541.1.3] tyrosine, preferably the L-enantiomer of tyrosine, its salts, ester or amides in free form or bound to proteins. In a preferred embodiment, the term “the fine chemical” means tyrosine or its salts, in free form or bound to proteins. In a preferred embodiment, the term “the fine chemical” means the L-enantiomer of tyrosine.

On the other hand in case “tyrosine” is stated it means tyrosine itself, its salts, ester or amides in free form or bound to proteins, preferably the L-enantiomer of tyrosine, its salts, ester or amides in free form or bound to proteins. In a preferred embodiment “tyrosine” means the L-enantiomer of tyrosine in free form. In another preferred embodiment “tyrosine” means the L-enantiomer of tyrosine bound to proteins.

Further, the term “in context of any of the paragraphs [0014.1.3.3] to [0514.1.1.3]” as used herein means that for any of said paragraphs [0014.1.3.3] to [0514.1.1.3] the term “the fine chemical” is understood to follow the definition of section [0014.1.3.3] or section [0015.1.3.3], independently whether it refers to any other paragraph or not and whether the reference recites the term “fine chemical” in an other context.

Thus, in cases where one or more paragraphs or sections are incorporated by reference into any of the present paragraphs [0014.1.3.3] to [0514.1.1.3], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to this definition of this paragraph [0015.1.3.3].

Further, the term “fine chemicals” as used herein relates to compositions comprising said fine chemical(s), i.e. comprising 3,4-dihydroxyphenylalanine (dopa), 5-oxoproline, alanine, asparagine, aspartate, citrulline, glycine, homoserine, ornithine, phenylalanine, serine, and/or tyrosine, respectively.

Accordingly, the present invention relates to a process for the production of aspartate, which comprises
(a) increasing or generating one or more activities selected from the group consisting of 47266012-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of tyrosine, which comprises
(a) increasing or generating one or more activities selected from the group consisting of 47266012-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of 47266012-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of aspartate, which comprises
(a) increasing or generating one or more activities selected from the group consisting of 49747384_SOYBEAN-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of tyrosine, which comprises
(a) increasing or generating one or more activities selected from the group consisting of 49747384_SOYBEAN-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of 49747384_SOYBEAN-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of alanine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of citrulline, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of asparagine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of serine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of alanine, which comprises
(a) increasing or generating one or more activities selected from the group consisting of glucose-6-phosphate 1-dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating one or more activities selected from the group consisting of glucose-6-phosphate 1-dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of glucose-6-phosphate 1-dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating one or more activities selected from the group consisting of amino acid acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of citrulline, which comprises
(a) increasing or generating one or more activities selected from the group consisting of amino acid acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of phenylalanine, which comprises
(a) increasing or generating one or more activities selected from the group consisting of amino acid acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of citrulline, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of glycine, which comprises
(a) increasing or generating one or more activities selected from the group consisting of DNA-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of serine, which comprises
(a) increasing or generating one or more activities selected from the group consisting of

DNA-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of phenylalanine, which comprises
(a) increasing or generating one or more activities selected from the group consisting of At1g17440-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of At1g17440-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating one or more activities selected from the group consisting of At1g19800-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of citrulline, which comprises
(a) increasing or generating one or more activities selected from the group consisting of At1g19800-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of tyrosine, which comprises
(a) increasing or generating one or more activities selected from the group consisting of At1g19800-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating one or more activities selected from the group consisting of cullin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of cullin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of homoserine, which comprises
(a) increasing or generating one or more activities selected from the group consisting of bifunctional aspartokinase/homoserine dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

Accordingly, the present invention relates to a process for the production of aspartate, which comprises
(a) increasing or generating one or more activities selected from the group consisting of eukaryotic translation initiation factor 5 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of eukaryotic translation initiation factor 5 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of eukaryotic translation initiation factor 5 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of eukaryotic translation initiation factor 5 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of eukaryotic translation initiation factor 5 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of phenylalanine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of glycine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of alanine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of glycine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of tyrosine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of alanine, which comprises
(a) increasing or generating one or more activities selected from the group consisting of aminotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of 5-oxoproline, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of asparagine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of phenylalanine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating one or more activities selected from the group consisting of CBL-interacting protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of CBL-interacting protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of CBL-interacting protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of serine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of glycine, which comprises
(a) increasing or generating one or more activities selected from the group consisting of CBL-interacting protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of alanine, which comprises
(a) increasing or generating one or more activities selected from the group consisting of delta-1-pyrroline 5-carboxylase synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of glycine, which comprises
(a) increasing or generating one or more activities selected from the group consisting of delta-1-pyrroline 5-carboxylase synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of tyrosine, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating one or more activities selected from the group consisting of ankyrin repeat family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating one or more activities selected from the group consisting of thioredoxin family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of thioredoxin family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of thioredoxin family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating one or more activities selected from the group consisting of harpin-induced family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of harpin-induced family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of harpin-induced family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating one or more activities selected from the group consisting of DNA mismatch repair protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of phenylalanine, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of serine, which comprises
(a) increasing or generating one or more activities selected from the group consisting of eukaryotic translation initiation factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of glucose-6-phosphate 1-dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating one or more activities selected from the group consisting of glycosy) transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of glycosyl transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating one or more activities selected from the group consisting of auxin response factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of auxin response factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating one or more activities selected from the group consisting of monthiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of monthiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of phenylalanine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of phenylalanine, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of tyrosine, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of tyrosine, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of serine, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of alanine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of aspartate, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of citrulline, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of alanine, which comprises
(a) increasing or generating one or more activities selected from the group consisting of At4g32480-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of asparagine, which comprises
(a) increasing or generating one or more activities selected from the group consisting of At4g32480-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of glycine, which comprises
(a) increasing or generating one or more activities selected from the group consisting of At4g32480-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating one or more activities selected from the group consisting of At4g32480-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of At4g32480-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of At4g32480-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of citrulline, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of phenylalanine, which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of asparagine, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of phenylalanine, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of tyrosine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of 3,4-dihydroxyphenylalanine (dopa), which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4-dihydroxyphenylalanine (dopa) or a composition comprising 3,4-dihydroxyphenylalanine (dopa) in said non-human organism or in the culture medium surrounding said non-human organism.

Accordingly, the present invention relates to a process for the production of phenylalanine, which comprises
(a) increasing or generating one or more activities selected from the group consisting of gibberellin 20-oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of gibberellin 20-oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of serine, which comprises
(a) increasing or generating one or more activities selected from the group consisting of integral membrane transporter family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of phenylalanine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of asparagine, which comprises
(a) increasing or generating one or more activities selected from the group consisting of At5g16650-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of aspartate, which comprises
(a) increasing or generating one or more activities selected from the group consisting of At5g16650-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of serine, which comprises
(a) increasing or generating one or more activities selected from the group consisting of At5g16650-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of 5-oxoproline, which comprises
(a) increasing or generating one or more activities selected from the group consisting of At5g16650-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of phenylalanine, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating one or more activities selected from the group consisting of at5g21910-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of serine, which comprises
(a) increasing or generating one or more activities selected from the group consisting of thioredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of alanine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of citrulline, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of citrulline, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of phenylalanine, which comprises
(a) increasing or generating one or more activities selected from the group consisting of RNA-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of RNA-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of alanine, which comprises
(a) increasing or generating one or more activities selected from the group consisting of zinc finger protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating one or more activities selected from the group consisting of zinc finger protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of zinc finger protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of 5-oxoproline, which comprises
(a) increasing or generating one or more activities selected from the group consisting of zinc finger protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of aspartate, which comprises
(a) increasing or generating one or more activities selected from the group consisting of

CDP-diacylglycerol-serine 0-phosphatidyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of 5-oxoproline, which comprises
(a) increasing or generating one or more activities selected from the group consisting of

CDP-diacylglycerol-serine 0-phosphatidyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of serine, which comprises
(a) increasing or generating one or more activities selected from the group consisting of Glycine cleavage system T aminomethyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of serine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating one or more activities selected from the group consisting of adenylylsulfate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of alanine, which comprises
(a) increasing or generating one or more activities selected from the group consisting of malic enzyme in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating one or more activities selected from the group consisting of malic enzyme in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of malic enzyme in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of alanine, which comprises
(a) increasing or generating one or more activities selected from the group consisting of sec-independent protein translocase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of alanine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of alanine, which comprises
(a) increasing or generating one or more activities selected from the group consisting of beta-hydroxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of aspartate, which comprises
(a) increasing or generating one or more activities selected from the group consisting of beta-hydroxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of serine, which comprises
(a) increasing or generating one or more activities selected from the group consisting of beta-hydroxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating one or more activities selected from the group consisting of elongation factor Tu in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of elongation factor Tu in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of elongation factor Tu in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of elongation factor Tu in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of elongation factor Tu in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of tyrosine, which comprises
(a) increasing or generating one or more activities selected from the group consisting of 30S ribosomal protein S3 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of asparagine, which comprises
(a) increasing or generating one or more activities selected from the group consisting of 50S ribosomal protein L14 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of tyrosine, which comprises
(a) increasing or generating one or more activities selected from the group consisting of ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating one or more activities selected from the group consisting of acyl-CoA synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of tyrosine, which comprises
(a) increasing or generating one or more activities selected from the group consisting of acyl-CoA synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of acyl-CoA synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of aspartate, which comprises
(a) increasing or generating one or more activities selected from the group consisting of 2-oxoglutarate dehydrogenase E1 subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of 5-oxoproline, which comprises
(a) increasing or generating one or more activities selected from the group consisting of 2-oxoglutarate dehydrogenase E1 subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of serine, which comprises
(a) increasing or generating one or more activities selected from the group consisting of

GDP-mannose dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of serine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of phenylalanine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of aspartate, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of tyrosine, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of citrulline, which comprises
(a) increasing or generating one or more activities selected from the group consisting of lysyl-tRNA synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of asparagine, which comprises
(a) increasing or generating one or more activities selected from the group consisting of ABC transporter component in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of aspartate, which comprises
(a) increasing or generating one or more activities selected from the group consisting of glucose-1-phosphate cytidylyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of glycine, which comprises
(a) increasing or generating one or more activities selected from the group consisting of CDP-glucose-4,6-dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating one or more activities selected from the group consisting of enoyl-CoA hydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of alanine, which comprises
(a) increasing or generating one or more activities selected from the group consisting of glutathione peroxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of asparagine, which comprises
(a) increasing or generating one or more activities selected from the group consisting of aminotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of phenylalanine, which comprises
(a) increasing or generating one or more activities selected from the group consisting of aminotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of NADH-quinone oxidoreductase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating one or more activities selected from the group consisting of glutamate-ammonia-ligase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of glutamate-ammonia-ligase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of RNA polymerase sigma factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of phenylalanine, which comprises
(a) increasing or generating one or more activities selected from the group consisting of hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of aspartate, which comprises
(a) increasing or generating one or more activities selected from the group consisting of glutamate-ammonia-ligase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of serine, which comprises
(a) increasing or generating one or more activities selected from the group consisting of glutamate-ammonia-ligase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of glutamate-ammonia-ligase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating one or more activities selected from the group consisting of fumarylacetoacetate hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of fumarylacetoacetate hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of serine, which comprises
(a) increasing or generating one or more activities selected from the group consisting of HesB/YadR/YfhF family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating one or more activities selected from the group consisting of peptidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of aspartate, which comprises
(a) increasing or generating one or more activities selected from the group consisting of L-aspartate oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of

Chaperone protein CIpB in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of phenylalanine, which comprises
(a) increasing or generating one or more activities selected from the group consisting of electron transfer flavoprotein subunit beta in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of electron transfer flavoprotein subunit beta in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of citrulline, which comprises
(a) increasing or generating one or more activities selected from the group consisting of AX653549-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of 5-oxoproline, which comprises
(a) increasing or generating one or more activities selected from the group consisting of AX653549-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of aspartate, which comprises
(a) increasing or generating one or more activities selected from the group consisting of AY087308-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of 5-oxoproline, which comprises
(a) increasing or generating one or more activities selected from the group consisting of AY087308-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of alanine, which comprises
(a) increasing or generating one or more activities selected from the group consisting of threonine synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating one or more activities selected from the group consisting of threonine synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of threonine synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of threonine synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of threonine synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of phenylalanine, which comprises
(a) increasing or generating one or more activities selected from the group consisting of acetolactate synthase small subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of glycine, which comprises
(a) increasing or generating one or more activities selected from the group consisting of glucose dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating one or more activities selected from the group consisting of glucose dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of glucose dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of glucose dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of asparagine, which comprises
(a) increasing or generating one or more activities selected from the group consisting of aspartate 1-decarboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of serine, which comprises
(a) increasing or generating one or more activities selected from the group consisting of aspartate 1-decarboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of alanine, which comprises
(a) increasing or generating one or more activities selected from the group consisting of serine protease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of serine, which comprises
(a) increasing or generating one or more activities selected from the group consisting of serine protease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of serine protease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of serine, which comprises
(a) increasing or generating one or more activities selected from the group consisting of acyl-CoA dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of acyl-CoA dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of tyrosine, which comprises
(a) increasing or generating one or more activities selected from the group consisting of beta-galactosidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of beta-galactosidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of glycine, which comprises
(a) increasing or generating one or more activities selected from the group consisting of 2,3-dihydroxyphenylpropionate 1,2-dioxygenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating one or more activities selected from the group consisting of 2,3-dihydroxyphenylpropionate 1,2-dioxygenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of 2,3-dihydroxyphenylpropionate 1,2-dioxygenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of 2,3-dihydroxyphenylpropionate 1,2-dioxygenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating one or more activities selected from the group consisting of b0391-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of asparagine, which comprises
(a) increasing or generating one or more activities selected from the group consisting of ATP-binding component of a transport system in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of aspartate, which comprises
(a) increasing or generating one or more activities selected from the group consisting of ATP-binding component of a transport system in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of ATP-binding component of a transport system in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of ATP-binding component of a transport system in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of ATP-binding component of a transport system in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating one or more activities selected from the group consisting of b0456-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of aspartate, which comprises
(a) increasing or generating one or more activities selected from the group consisting of membrane transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of membrane transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of membrane transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of homoserine, which comprises
(a) increasing or generating one or more activities selected from the group consisting of b0518-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

Accordingly, the present invention relates to a process for the production of aspartate, which comprises
(a) increasing or generating one or more activities selected from the group consisting of isochorismate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of isochorismate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of isochorismate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of isochorismate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 3,4-dihydroxyphenylalanine (dopa), which comprises
(a) increasing or generating one or more activities selected from the group consisting of isochorismate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4-dihydroxyphenylalanine (dopa) or a composition comprising 3,4-dihydroxyphenylalanine (dopa) in said non-human organism or in the culture medium surrounding said non-human organism.

Accordingly, the present invention relates to a process for the production of 5-oxoproline, which comprises
(a) increasing or generating one or more activities selected from the group consisting of isochorismate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of tyrosine, which comprises
(a) increasing or generating one or more activities selected from the group consisting of zinc transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of tyrosine, which comprises
(a) increasing or generating one or more activities selected from the group consisting of 3-deoxy-D-arabinoheptulosonate-7-phosphatesynthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of tyrosine, which comprises
(a) increasing or generating one or more activities selected from the group consisting of b0801-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating one or more activities selected from the group consisting of asparaginase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of glycine, which comprises
(a) increasing or generating one or more activities selected from the group consisting of b0841-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of homoserine, which comprises
(a) increasing or generating one or more activities selected from the group consisting of b0841-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

Accordingly, the present invention relates to a process for the production of phenylalanine, which comprises
(a) increasing or generating one or more activities selected from the group consisting of major facilitator superfamily transporter protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of major facilitator superfamily transporter protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of major facilitator superfamily transporter protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of major facilitator superfamily transporter protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of citrulline, which comprises
(a) increasing or generating one or more activities selected from the group consisting of b0917-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of glycine, which comprises
(a) increasing or generating one or more activities selected from the group consisting of b0917-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating one or more activities selected from the group consisting of b0917-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of 5-oxoproline, which comprises
(a) increasing or generating one or more activities selected from the group consisting of b0917-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of alanine, which comprises
(a) increasing or generating one or more activities selected from the group consisting of methylglyoxal synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating one or more activities selected from the group consisting of methylglyoxal synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of methylglyoxal synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating one or more activities selected from the group consisting of phosphoanhydride phosphorylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of alanine, which comprises
(a) increasing or generating one or more activities selected from the group consisting of b1003-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of aspartate, which comprises
(a) increasing or generating one or more activities selected from the group consisting of b1003-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of b1003-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of b1003-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of b1003-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 3,4-dihydroxyphenylalanine (dopa), which comprises
(a) increasing or generating one or more activities selected from the group consisting of b1003-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4-dihydroxyphenylalanine (dopa) or a composition comprising 3,4-dihydroxyphenylalanine (dopa) in said non-human organism or in the culture medium surrounding said non-human organism.

Accordingly, the present invention relates to a process for the production of 5-oxoproline, which comprises
(a) increasing or generating one or more activities selected from the group consisting of b1003-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of citrulline, which comprises
(a) increasing or generating one or more activities selected from the group consisting of lipoprotein precursor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of 5-oxoproline, which comprises
(a) increasing or generating one or more activities selected from the group consisting of lipoprotein precursor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of aspartate, which comprises
(a) increasing or generating one or more activities selected from the group consisting of b1024-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of aspartate, which comprises
(a) increasing or generating one or more activities selected from the group consisting of multidrug resistance protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of tyrosine, which comprises
(a) increasing or generating one or more activities selected from the group consisting of multidrug resistance protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of serine, which comprises
(a) increasing or generating one or more activities selected from the group consisting of b1137-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of phenylalanine, which comprises
(a) increasing or generating one or more activities selected from the group consisting of b1155-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of phenylalanine, which comprises
(a) increasing or generating one or more activities selected from the group consisting of b1163-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of b1163-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of citrulline, which comprises
(a) increasing or generating one or more activities selected from the group consisting of b1179-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of alanine, which comprises
(a) increasing or generating one or more activities selected from the group consisting of sodium/proton antiporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of serine, which comprises
(a) increasing or generating one or more activities selected from the group consisting of sodium/proton antiporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of citrulline, which comprises
(a) increasing or generating one or more activities selected from the group consisting of b1205-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of phenylalanine, which comprises
(a) increasing or generating one or more activities selected from the group consisting of b1229-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of membrane protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of citrulline, which comprises
(a) increasing or generating one or more activities selected from the group consisting of b1259-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of 5-oxoproline, which comprises
(a) increasing or generating one or more activities selected from the group consisting of b1259-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of tyrosine, which comprises
(a) increasing or generating one or more activities selected from the group consisting of tryptophan biosynthesis protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating one or more activities selected from the group consisting of anthranilate synthase component I in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of glycine, which comprises
(a) increasing or generating one or more activities selected from the group consisting of b1280-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of serine, which comprises
(a) increasing or generating one or more activities selected from the group consisting of b1280-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of 5-oxoproline, which comprises
(a) increasing or generating one or more activities selected from the group consisting of b1280-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of alanine, which comprises
(a) increasing or generating one or more activities selected from the group consisting of glutamine synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of glycine, which comprises
(a) increasing or generating one or more activities selected from the group consisting of glutamine synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating one or more activities selected from the group consisting of aldehyde dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of aldehyde dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of citrulline, which comprises
(a) increasing or generating one or more activities selected from the group consisting of ABC transporter substrate-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of citrulline, which comprises
(a) increasing or generating one or more activities selected from the group consisting of b1330-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of phenylalanine, which comprises
(a) increasing or generating one or more activities selected from the group consisting of ATPdependent RNA helicase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of alanine, which comprises
(a) increasing or generating one or more activities selected from the group consisting of restriction alleviation protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of glycine, which comprises
(a) increasing or generating one or more activities selected from the group consisting of restriction alleviation protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of 5-oxoproline, which comprises
(a) increasing or generating one or more activities selected from the group consisting of lipoprotein precursor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of alanine, which comprises
(a) increasing or generating one or more activities selected from the group consisting of b1445-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of citrulline, which comprises
(a) increasing or generating one or more activities selected from the group consisting of b1445-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of glycine, which comprises
(a) increasing or generating one or more activities selected from the group consisting of b1445-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating one or more activities selected from the group consisting of b1445-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of b1445-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of alanine, which comprises
(a) increasing or generating one or more activities selected from the group consisting of NADP-dependent malic enzyme in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of glycine, which comprises
(a) increasing or generating one or more activities selected from the group consisting of NADP-dependent malic enzyme in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating one or more activities selected from the group consisting of NADP-dependent malic enzyme in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of NADP-dependent malic enzyme in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of NADP-dependent malic enzyme in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of asparagine, which comprises
(a) increasing or generating one or more activities selected from the group consisting of b1522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of aspartate, which comprises
(a) increasing or generating one or more activities selected from the group consisting of b1522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of b1522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of b1522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of b1522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of b1522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of tyrosine, which comprises
(a) increasing or generating one or more activities selected from the group consisting of b1583-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating one or more activities selected from the group consisting of Fe—S subunit of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of acid shock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of acid shock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating one or more activities selected from the group consisting of methylglyoxalase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of aspartate, which comprises
(a) increasing or generating one or more activities selected from the group consisting of b1670-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of aspartate, which comprises
(a) increasing or generating one or more activities selected from the group consisting of b1672-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of alanine, which comprises
(a) increasing or generating one or more activities selected from the group consisting of arginine N-succinyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of aspartate, which comprises
(a) increasing or generating one or more activities selected from the group consisting of ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of tyrosine, which comprises
(a) increasing or generating one or more activities selected from the group consisting of ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating one or more activities selected from the group consisting of transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of aspartate, which comprises
(a) increasing or generating one or more activities selected from the group consisting of b1837-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of aspartate, which comprises
(a) increasing or generating one or more activities selected from the group consisting of serine/threonine-protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of aspartate, which comprises
(a) increasing or generating one or more activities selected from the group consisting of protease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of serine, which comprises
(a) increasing or generating one or more activities selected from the group consisting of protease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of asparagine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of tyrosine, which comprises
(a) increasing or generating one or more activities selected from the group consisting of cytochrome c-type protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating one or more activities selected from the group consisting of trehalose-phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of trehalose-phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of serine, which comprises
(a) increasing or generating one or more activities selected from the group consisting of b1898-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of serine, which comprises
(a) increasing or generating one or more activities selected from the group consisting of b1930-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating one or more activities selected from the group consisting of transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of tyrosine, which comprises
(a) increasing or generating one or more activities selected from the group consisting of O-antigen chain length determinant in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating one or more activities selected from the group consisting of b2032-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of aspartate, which comprises
(a) increasing or generating one or more activities selected from the group consisting of transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of uridine/cytidine kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of aspartate, which comprises
(a) increasing or generating one or more activities selected from the group consisting of b2107-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of aspartate, which comprises
(a) increasing or generating one or more activities selected from the group consisting of ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of citrulline, which comprises
(a) increasing or generating one or more activities selected from the group consisting of ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of phenylalanine, which comprises
(a) increasing or generating one or more activities selected from the group consisting of ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of glycine, which comprises
(a) increasing or generating one or more activities selected from the group consisting of sn-glycerol-3-phosphate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of asparagine, which comprises
(a) increasing or generating one or more activities selected from the group consisting of NADH dehydrogenase I chain I in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of aspartate, which comprises
(a) increasing or generating one or more activities selected from the group consisting of b2360-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of serine, which comprises
(a) increasing or generating one or more activities selected from the group consisting of D-serine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of b2399-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 3,4-dihydroxyphenylalanine (dopa), which comprises
(a) increasing or generating one or more activities selected from the group consisting of b2399-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4-dihydroxyphenylalanine (dopa) or a composition comprising 3,4-dihydroxyphenylalanine (dopa) in said non-human organism or in the culture medium surrounding said non-human organism.

Accordingly, the present invention relates to a process for the production of 5-oxoproline, which comprises
(a) increasing or generating one or more activities selected from the group consisting of b2399-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of alanine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of aspartate, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of PTS system N-acetylmuramic acid-specific EIIBC component in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of citrulline, which comprises
(a) increasing or generating one or more activities selected from the group consisting of b2474-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of glycine, which comprises
(a) increasing or generating one or more activities selected from the group consisting of b2474-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of serine, which comprises
(a) increasing or generating one or more activities selected from the group consisting of NADH dehydrogenase subunit N in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of 5-oxoproline, which comprises
(a) increasing or generating one or more activities selected from the group consisting of NADH dehydrogenase subunit N in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of phenylalanine, which comprises
(a) increasing or generating one or more activities selected from the group consisting of b2513-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of b2513-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of serine, which comprises
(a) increasing or generating one or more activities selected from the group consisting of serine hydroxymethyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of phenylalanine, which comprises
(a) increasing or generating one or more activities selected from the group consisting of chorismate mutase-T and prephenate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of alanine, which comprises
(a) increasing or generating one or more activities selected from the group consisting of b2613-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating one or more activities selected from the group consisting of b2673-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of b2673-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating one or more activities selected from the group consisting of transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of alanine, which comprises
(a) increasing or generating one or more activities selected from the group consisting of murein transglycosylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating one or more activities selected from the group consisting of murein transglycosylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of murein transglycosylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of alanine, which comprises
(a) increasing or generating one or more activities selected from the group consisting of b2739-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating one or more activities selected from the group consisting of b2739-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of b2739-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of tyrosine, which comprises
(a) increasing or generating one or more activities selected from the group consisting of 2-C-methyl-D-erythritol 4-phosphate cytidylyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of 2-C-methyl-D-erythritol 4-phosphate cytidylyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of phenylalanine, which comprises
(a) increasing or generating one or more activities selected from the group consisting of phosphoadenosine phosphosulfate reductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of phenylalanine, which comprises
(a) increasing or generating one or more activities selected from the group consisting of b2812-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of b2812-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of b2812-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating one or more activities selected from the group consisting of amino-acid acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of amino-acid acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of amino-acid acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of aspartate, which comprises
(a) increasing or generating one or more activities selected from the group consisting of prolipoprotein diacylglyceryl transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of tyrosine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating one or more activities selected from the group consisting of b2849-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of glycine, which comprises
(a) increasing or generating one or more activities selected from the group consisting of D-3-phosphoglycerate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of serine, which comprises
(a) increasing or generating one or more activities selected from the group consisting of D-3-phosphoglycerate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating one or more activities selected from the group consisting of arginine exporter protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of asparagine, which comprises
(a) increasing or generating one or more activities selected from the group consisting of glycoprotease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of phenylalanine, which comprises
(a) increasing or generating one or more activities selected from the group consisting of b3083-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of b3083-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of b3083-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating one or more activities selected from the group consisting of b3098-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of glycine, which comprises
(a) increasing or generating one or more activities selected from the group consisting of b3098-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of tyrosine, which comprises
(a) increasing or generating one or more activities selected from the group consisting of b3121-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of tyrosine, which comprises
(a) increasing or generating one or more activities selected from the group consisting of b3221-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of serine, which comprises
(a) increasing or generating one or more activities selected from the group consisting of b3246-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of 5-oxoproline, which comprises
(a) increasing or generating one or more activities selected from the group consisting of b3246-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of asparagine, which comprises
(a) increasing or generating one or more activities selected from the group consisting of acetyl CoA carboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of glycine, which comprises
(a) increasing or generating one or more activities selected from the group consisting of acetyl CoA carboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of serine, which comprises
(a) increasing or generating one or more activities selected from the group consisting of acetyl CoA carboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of 5-oxoproline, which comprises
(a) increasing or generating one or more activities selected from the group consisting of acetyl CoA carboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of alanine, which comprises
(a) increasing or generating one or more activities selected from the group consisting of methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating one or more activities selected from the group consisting of methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of alanine, which comprises
(a) increasing or generating one or more activities selected from the group consisting of b3346-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of alanine, which comprises
(a) increasing or generating one or more activities selected from the group consisting of ABC transporter ATP-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of glycine, which comprises
(a) increasing or generating one or more activities selected from the group consisting of ABC transporter ATP-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of aspartate, which comprises
(a) increasing or generating one or more activities selected from the group consisting of b3392-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of phosphoenolpyruvate carboxykinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of phosphoenolpyruvate carboxykinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of alanine, which comprises
(a) increasing or generating one or more activities selected from the group consisting of b3410-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of serine, which comprises
(a) increasing or generating one or more activities selected from the group consisting of b3509-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating one or more activities selected from the group consisting of cellulose synthase catalytic subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of serine acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of serine acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating one or more activities selected from the group consisting of phosphopantetheine adenylyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of asparagine, which comprises
(a) increasing or generating one or more activities selected from the group consisting of phosphopantetheine adenylyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of phenylalanine, which comprises
(a) increasing or generating one or more activities selected from the group consisting of phosphopantetheine adenylyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of glycine, which comprises
(a) increasing or generating one or more activities selected from the group consisting of b3646-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of tyrosine, which comprises
(a) increasing or generating one or more activities selected from the group consisting of acetolactate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of asparagine, which comprises
(a) increasing or generating one or more activities selected from the group consisting of aspartate-ammonia ligase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of glycine, which comprises
(a) increasing or generating one or more activities selected from the group consisting of dihydroxyacid dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating one or more activities selected from the group consisting of dihydroxyacid dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of aspartate, which comprises
(a) increasing or generating one or more activities selected from the group consisting of b3791-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of 5-oxoproline, which comprises
(a) increasing or generating one or more activities selected from the group consisting of b3791-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of phenylalanine, which comprises
(a) increasing or generating one or more activities selected from the group consisting of enterobacterial common antigen polymerase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of phenylalanine, which comprises
(a) increasing or generating one or more activities selected from the group consisting of DNA helicase II in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of citrulline, which comprises
(a) increasing or generating one or more activities selected from the group consisting of b3814-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of alanine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of aspartate, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of serine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating one or more activities selected from the group consisting of b3989-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of b3989-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of 5-oxoproline, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of glycine, which comprises
(a) increasing or generating one or more activities selected from the group consisting of aspartate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of serine, which comprises
(a) increasing or generating one or more activities selected from the group consisting of aspartate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of alanine, which comprises
(a) increasing or generating one or more activities selected from the group consisting of b4029-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of aspartate, which comprises
(a) increasing or generating one or more activities selected from the group consisting of b4029-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of glycine, which comprises
(a) increasing or generating one or more activities selected from the group consisting of b4029-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating one or more activities selected from the group consisting of b4029-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of b4029-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of b4029-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of b4029-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of serine, which comprises
(a) increasing or generating one or more activities selected from the group consisting of b4121-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of serine, which comprises
(a) increasing or generating one or more activities selected from the group consisting of thioredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of alanine, which comprises
(a) increasing or generating one or more activities selected from the group consisting of lysine decarboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of asparagine, which comprises
(a) increasing or generating one or more activities selected from the group consisting of GM02LC11114-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of phenylalanine, which comprises
(a) increasing or generating one or more activities selected from the group consisting of GM02LC11114-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of alanine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating one or more activities selected from the group consisting of histone H2A in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of histone H2A in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating one or more activities selected from the group consisting of GM02LC17485-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of RNA binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of RNA binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of serine, which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of alanine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of glycine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of serine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of asparagine, which comprises
(a) increasing or generating one or more activities selected from the group consisting of s_pp015018333r-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of phenylalanine, which comprises
(a) increasing or generating one or more activities selected from the group consisting of s_pp015018333r-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of s_pp015018333r-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of s_pp015018333r-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of citrulline, which comprises
(a) increasing or generating one or more activities selected from the group consisting of N-acetyl-gamma-glutamyl-phosphate reductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of asparagine, which comprises
(a) increasing or generating one or more activities selected from the group consisting of agmatinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of tyrosine, which comprises
(a) increasing or generating one or more activities selected from the group consisting of agmatinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating one or more activities selected from the group consisting of ABC transporter ATP binding component in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating one or more activities selected from the group consisting of flavodoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of flavodoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating one or more activities selected from the group consisting of sll0281-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of serine, which comprises
(a) increasing or generating one or more activities selected from the group consisting of uroporphyrin-III C-methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of phenylalanine, which comprises
(a) increasing or generating one or more activities selected from the group consisting of urease subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of urease subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of citrulline, which comprises
(a) increasing or generating one or more activities selected from the group consisting of glucokinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of serine, which comprises
(a) increasing or generating one or more activities selected from the group consisting of permease protein of phosphate ABC transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of 5-oxoproline, which comprises
(a) increasing or generating one or more activities selected from the group consisting of permease protein of phosphate ABC transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of aspartate, which comprises
(a) increasing or generating one or more activities selected from the group consisting of oxireductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of glycine, which comprises
(a) increasing or generating one or more activities selected from the group consisting of oxireductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of serine, which comprises
(a) increasing or generating one or more activities selected from the group consisting of oxireductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of oxireductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating one or more activities selected from the group consisting of malate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of malate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of malate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating one or more activities selected from the group consisting of 3-deoxy-7-phosphoheptulonate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of serine, which comprises
(a) increasing or generating one or more activities selected from the group consisting of glycogen synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating one or more activities selected from the group consisting of carbon dioxide concentrating mechanism protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of carbon dioxide concentrating mechanism protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of carbon dioxide concentrating mechanism protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating one or more activities selected from the group consisting of sll1032-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of serine, which comprises
(a) increasing or generating one or more activities selected from the group consisting of phosphoribosylformyl glycinamidine synthase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating one or more activities selected from the group consisting of 5′-nucleotidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of 5-oxoproline, which comprises
(a) increasing or generating one or more activities selected from the group consisting of 5′-nucleotidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of asparagine, which comprises
(a) increasing or generating one or more activities selected from the group consisting of glycogen (starch) synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of tyrosine, which comprises
(a) increasing or generating one or more activities selected from the group consisting of glycogen (starch) synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating one or more activities selected from the group consisting of CTP synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of glycine, which comprises
(a) increasing or generating one or more activities selected from the group consisting of nitrate/nitrite transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating one or more activities selected from the group consisting of nitrate/nitrite transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of nitrate/nitrite transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of nitrate/nitrite transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating one or more activities selected from the group consisting of CDP-diacylglycerol-glycerol-3-phosphate 3-phosphatidyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of 5-oxoproline, which comprises
(a) increasing or generating one or more activities selected from the group consisting of

CDP-diacylglycerol-glycerol-3-phosphate 3-phosphatidyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of serine, which comprises
(a) increasing or generating one or more activities selected from the group consisting of glutathione S-transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating one or more activities selected from the group consisting of exopolyphosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of exopolyphosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of exopolyphosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating one or more activities selected from the group consisting of ABC metal ion transporter substrate-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of ABC metal ion transporter substrate-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of sll1761-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of sll1761-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating one or more activities selected from the group consisting of adenylate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of citrulline, which comprises
(a) increasing or generating one or more activities selected from the group consisting of adenylate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of glycine, which comprises
(a) increasing or generating one or more activities selected from the group consisting of adenylate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of serine, which comprises
(a) increasing or generating one or more activities selected from the group consisting of adenylate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of adenylate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of glycine, which comprises
(a) increasing or generating one or more activities selected from the group consisting of acyl transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating one or more activities selected from the group consisting of acyl transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of acyl transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating one or more activities selected from the group consisting of coproporphyrinogen III oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of citrulline, which comprises
(a) increasing or generating one or more activities selected from the group consisting of coproporphyrinogen III oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of phenylalanine, which comprises
(a) increasing or generating one or more activities selected from the group consisting of coproporphyrinogen III oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of coproporphyrinogen III oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of coproporphyrinogen Ill oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating one or more activities selected from the group consisting of cation-transporting ATPase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of aspartate, which comprises
(a) increasing or generating one or more activities selected from the group consisting of cation-transporting ATPase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of glycine, which comprises
(a) increasing or generating one or more activities selected from the group consisting of glycosyl transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating one or more activities selected from the group consisting of precorrin methylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of precorrin methylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of citrulline, which comprises
(a) increasing or generating one or more activities selected from the group consisting of aldehyde dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of alanine, which comprises
(a) increasing or generating one or more activities selected from the group consisting of bifunctional purine biosynthesis protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of serine, which comprises
(a) increasing or generating one or more activities selected from the group consisting of bifunctional purine biosynthesis protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of 5-oxoproline, which comprises
(a) increasing or generating one or more activities selected from the group consisting of bifunctional purine biosynthesis protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of asparagine, which comprises
(a) increasing or generating one or more activities selected from the group consisting of Glu/Leu/Phe/Val dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of phenylalanine, which comprises
(a) increasing or generating one or more activities selected from the group consisting of Glu/Leu/Phe/Val dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of Glu/Leu/Phe/Val dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating one or more activities selected from the group consisting of malate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of alanine, which comprises
(a) increasing or generating one or more activities selected from the group consisting of geranylgeranyl pyrophosphate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of asparagine, which comprises
(a) increasing or generating one or more activities selected from the group consisting of geranylgeranyl pyrophosphate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of phenylalanine, which comprises
(a) increasing or generating one or more activities selected from the group consisting of geranylgeranyl pyrophosphate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of geranylgeranyl pyrophosphate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of geranylgeranyl pyrophosphate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of geranylgeranyl pyrophosphate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of phenylalanine, which comprises
(a) increasing or generating one or more activities selected from the group consisting of amine oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of tryptophan synthase alpha chain in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of ornithine, which comprises
(a) increasing or generating one or more activities selected from the group consisting of argininosuccinate lyase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of ornithine or a composition comprising ornithine in said non-human organism or in the culture medium surrounding said non-human organism.

Accordingly, the present invention relates to a process for the production of serine, which comprises
(a) increasing or generating one or more activities selected from the group consisting of argininosuccinate lyase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of asparagine, which comprises
(a) increasing or generating one or more activities selected from the group consisting of gamma-glutamyltranspeptidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of aspartate, which comprises
(a) increasing or generating one or more activities selected from the group consisting of iron(III) dicitrate-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of iron(III) dicitrate-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of phenylalanine, which comprises
(a) increasing or generating one or more activities selected from the group consisting of Na+/K+ transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of Na+/K+ transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating one or more activities selected from the group consisting of 3-oxoacyl-[acyl-carrier-protein] synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of 3-oxoacyl-[acyl-carrier-protein] synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of phenylalanine, which comprises
(a) increasing or generating one or more activities selected from the group consisting of Photosystem I reaction center subunit XI in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of Photosystem I reaction center subunit XI in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of Photosystem I reaction center subunit XI in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating one or more activities selected from the group consisting of photosystem II protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of photosystem II protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of photosystem II protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of photosystem II protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of asparagine, which comprises
(a) increasing or generating one or more activities selected from the group consisting of glutamine amidotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of citrulline, which comprises
(a) increasing or generating one or more activities selected from the group consisting of phosphoadenosine phosphosulfate reductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of asparagine, which comprises
(a) increasing or generating one or more activities selected from the group consisting of riboflavin biosynthesis protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of homoserine, which comprises
(a) increasing or generating one or more activities selected from the group consisting of threonine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

Accordingly, the present invention relates to a process for the production of tyrosine, which comprises
(a) increasing or generating one or more activities selected from the group consisting of threonine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating one or more activities selected from the group consisting of short-chain alcohol dehydrogenase family in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating one or more activities selected from the group consisting of short-chain alcohol dehydrogenase family in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of short-chain alcohol dehydrogenase family in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating one or more activities selected from the group consisting of Sec-independent protein translocase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of Sec-independent protein translocase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of Sec-independent protein translocase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of tyrosine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of asparagine, which comprises
(a) increasing or generating one or more activities selected from the group consisting of amino acid ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of tyrosine, which comprises
(a) increasing or generating one or more activities selected from the group consisting of TTC0768-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating one or more activities selected from the group consisting of TTC0881-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of serine, which comprises
(a) increasing or generating one or more activities selected from the group consisting of TTC0881-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of alanine, which comprises
(a) increasing or generating one or more activities selected from the group consisting of metal-dependent hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of serine, which comprises
(a) increasing or generating one or more activities selected from the group consisting of metal-dependent hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of phenylalanine, which comprises
(a) increasing or generating one or more activities selected from the group consisting of multiple antibiotic resistance protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of multiple antibiotic resistance protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating one or more activities selected from the group consisting of TTC1386-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of glycine, which comprises
(a) increasing or generating one or more activities selected from the group consisting of TTC1386-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating one or more activities selected from the group consisting of TTC1386-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of TTC1386-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of TCC1386-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating one or more activities selected from the group consisting of homocitrate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating one or more activities selected from the group consisting of homocitrate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of homocitrate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of homocitrate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of homocitrate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of aspartate, which comprises
(a) increasing or generating one or more activities selected from the group consisting of oxidoreductase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of serine, which comprises
(a) increasing or generating one or more activities selected from the group consisting of oxidoreductase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating one or more activities selected from the group consisting of XM_{—}473199-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of XM_{—}473199-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of XM_{—}473199-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of XM_{—}473199-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of glycine, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of aspartate, which comprises
(a) increasing or generating one or more activities selected from the group consisting of cell division control protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of tyrosine, which comprises
(a) increasing or generating one or more activities selected from the group consisting of cell division control protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating one or more activities selected from the group consisting of 3-deoxy-7-phosphoheptulonate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of 3-deoxy-7-phosphoheptulonate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of phosphate permease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of alanine, which comprises
(a) increasing or generating one or more activities selected from the group consisting of serine/threonine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of glycine, which comprises
(a) increasing or generating one or more activities selected from the group consisting of serine/threonine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of aspartate, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of homoserine, which comprises
(a) increasing or generating one or more activities selected from the group consisting of Serine/threonine-protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

Accordingly, the present invention relates to a process for the production of phenylalanine, which comprises
(a) increasing or generating one or more activities selected from the group consisting of Serine/threonine-protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of 5-oxoproline, which comprises
(a) increasing or generating one or more activities selected from the group consisting of Pre-mRNA-splicing factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of phenylalanine, which comprises
(a) increasing or generating one or more activities selected from the group consisting of N-(5′-phosphoribosyl)anthranilate isomerase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of branched-chain amino acid permease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of serine, which comprises
(a) increasing or generating one or more activities selected from the group consisting of ydr183w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating one or more activities selected from the group consisting of ydr273w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of citrulline, which comprises
(a) increasing or generating one or more activities selected from the group consisting of ydr273w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of aspartate, which comprises
(a) increasing or generating one or more activities selected from the group consisting of ydr507c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of asparagine, which comprises
(a) increasing or generating one or more activities selected from the group consisting of yer014w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of aspartate, which comprises
(a) increasing or generating one or more activities selected from the group consisting of yer014w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of serine, which comprises
(a) increasing or generating one or more activities selected from the group consisting of yer014w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of citrulline, which comprises
(a) increasing or generating one or more activities selected from the group consisting of yer106w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of serine, which comprises
(a) increasing or generating one or more activities selected from the group consisting of molecular chaperone portein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of molecular chaperone portein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating one or more activities selected from the group consisting of YFL019C-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of YFL019C-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of alanine, which comprises
(a) increasing or generating one or more activities selected from the group consisting of aminotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of glycine, which comprises
(a) increasing or generating one or more activities selected from the group consisting of aminotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating one or more activities selected from the group consisting of yfl054c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of yfl054c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of 5-oxoproline, which comprises
(a) increasing or generating one or more activities selected from the group consisting of ygl096w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of tyrosine, which comprises
(a) increasing or generating one or more activities selected from the group consisting of ygl237c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating one or more activities selected from the group consisting of ygr221 c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of 5-oxoproline, which comprises
(a) increasing or generating one or more activities selected from the group consisting of ygr221 c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of phenylalanine, which comprises
(a) increasing or generating one or more activities selected from the group consisting of yhl013c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of phenylalanine, which comprises
(a) increasing or generating one or more activities selected from the group consisting of yhr006w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of yhr006w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating one or more activities selected from the group consisting of yhr207c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of citrulline, which comprises
(a) increasing or generating one or more activities selected from the group consisting of 3-phosphoglycerate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of glycine, which comprises
(a) increasing or generating one or more activities selected from the group consisting of 3-phosphoglycerate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of aspartate, which comprises
(a) increasing or generating one or more activities selected from the group consisting of glutathione S-transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of citrulline, which comprises
(a) increasing or generating one or more activities selected from the group consisting of glutathione S-transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of aspartate, which comprises
(a) increasing or generating one or more activities selected from the group consisting of DnaJ-like chaperone in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of cystathionine gamma-synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of citrulline, which comprises
(a) increasing or generating one or more activities selected from the group consisting of branched chain aminotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of aspartate, which comprises
(a) increasing or generating one or more activities selected from the group consisting of polygalacturonase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of polygalacturonase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of alanine, which comprises
(a) increasing or generating one or more activities selected from the group consisting of YKL038W-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of aspartate, which comprises
(a) increasing or generating one or more activities selected from the group consisting of YKL038W-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of serine, which comprises
(a) increasing or generating one or more activities selected from the group consisting of YKL038W-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of glycine, which comprises
(a) increasing or generating one or more activities selected from the group consisting of aspartate aminotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of serine, which comprises
(a) increasing or generating one or more activities selected from the group consisting of thioredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of thioredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating one or more activities selected from the group consisting of sterol 24-C-methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of glycine, which comprises
(a) increasing or generating one or more activities selected from the group consisting of sterol 24-C-methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of homoserine, which comprises
(a) increasing or generating one or more activities selected from the group consisting of sterol 24-C-methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

Accordingly, the present invention relates to a process for the production of 5-oxoproline, which comprises
(a) increasing or generating one or more activities selected from the group consisting of sterol 24-C-methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of alanine, which comprises
(a) increasing or generating one or more activities selected from the group consisting of yml083c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating one or more activities selected from the group consisting of phosphate transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of phosphate transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of tyrosine, which comprises
(a) increasing or generating one or more activities selected from the group consisting of ymr013c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of glycine, which comprises
(a) increasing or generating one or more activities selected from the group consisting of purine biosynthesis protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating one or more activities selected from the group consisting of mitochondrial inner membrane protease subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of homoserine, which comprises
(a) increasing or generating one or more activities selected from the group consisting of YNL086W-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

Accordingly, the present invention relates to a process for the production of tyrosine, which comprises
(a) increasing or generating one or more activities selected from the group consisting of amino acid transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of homoserine, which comprises
(a) increasing or generating one or more activities selected from the group consisting of homoserine O-acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

Accordingly, the present invention relates to a process for the production of alanine, which comprises
(a) increasing or generating one or more activities selected from the group consisting of metal ion transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of aspartate, which comprises
(a) increasing or generating one or more activities selected from the group consisting of yol160w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of citrulline, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of phenylalanine, which comprises
(a) increasing or generating one or more activities selected from the group consisting of histone acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating one or more activities selected from the group consisting of histone acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of homoserine, which comprises
(a) increasing or generating one or more activities selected from the group consisting of diacylglycerol O-acyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

Accordingly, the present invention relates to a process for the production of serine, which comprises
(a) increasing or generating one or more activities selected from the group consisting of diacylglycerol O-acyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of 5-oxoproline, which comprises
(a) increasing or generating one or more activities selected from the group consisting of diacylglycerol O-acyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of glycine, which comprises
(a) increasing or generating one or more activities selected from the group consisting of acetyl-CoA acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of serine, which comprises
(a) increasing or generating one or more activities selected from the group consisting of citrate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating one or more activities selected from the group consisting of ammonium transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating one or more activities selected from the group consisting of Zm_{—}4842_BE510522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating one or more activities selected from the group consisting of Zm_{—}4842_BE510522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating one or more activities selected from the group consisting of Zm_{—}4842_BE510522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating one or more activities selected from the group consisting of Zm_{—}4842_BE510522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

An embodiment of the present invention relates to a process for the production of 3,4-dihydroxyphenylalanine (dopa), which comprises
(a1) increasing or generating the activity of a polypeptide comprising a polypeptide as depicted in a respective line in column 5 or 8 of Table II, application no. 3, or a homolog or a fragment thereof, a consensus sequence or at least one polypeptide motif as depicted in the respective line in column 8 of Table IV, application no. 3, whereby the respective line discloses in column 7 3,4-dihydroxyphenylalanine (dopa); or
(a2) increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, whereby the respective line discloses in column 7 3,4-dihydroxyphenylalanine (dopa); or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of 3,4-dihydroxyphenylalanine (dopa) or a composition comprising 3,4-dihydroxyphenylalanine (dopa) in said non-human organism or in the culture medium surrounding said non-human organism.

An embodiment of the present invention relates to a process for the production of 5-oxoproline, which comprises
(a1) increasing or generating the activity of a polypeptide comprising a polypeptide as depicted in a respective line in column 5 or 8 of Table II, application no. 3, or a homolog or a fragment thereof, a consensus sequence or at least one polypeptide motif as depicted in the respective line in column 8 of Table IV, application no. 3, whereby the respective line discloses in column 7 5-oxoproline; or
(a2) increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, whereby the respective line discloses in column 7 5-oxoproline; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of 5-oxoproline or a composition comprising 5-oxoproline in said non-human organism or in the culture medium surrounding said non-human organism.

An embodiment of the present invention relates to a process for the production of alanine, which comprises
(a1) increasing or generating the activity of a polypeptide comprising a polypeptide as depicted in a respective line in column 5 or 8 of Table II, application no. 3, or a homolog or a fragment thereof, a consensus sequence or at least one polypeptide motif as depicted in the respective line in column 8 of Table IV, application no. 3, whereby the respective line discloses in column 7 alanine; or
(a2) increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, whereby the respective line discloses in column 7 alanine; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of alanine or a composition comprising alanine in said non-human organism or in the culture medium surrounding said non-human organism.

An embodiment of the present invention relates to a process for the production of asparagine, which comprises
(a1) increasing or generating the activity of a polypeptide comprising a polypeptide as depicted in a respective line in column 5 or 8 of Table II, application no. 3, or a homolog or a fragment thereof, a consensus sequence or at least one polypeptide motif as depicted in the respective line in column 8 of Table IV, application no. 3, whereby the respective line discloses in column 7 asparagine; or
(a2) increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, whereby the respective line discloses in column 7 asparagine; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of asparagine or a composition comprising asparagine in said non-human organism or in the culture medium surrounding said non-human organism.

An embodiment of the present invention relates to a process for the production of aspartate, which comprises
(a1) increasing or generating the activity of a polypeptide comprising a polypeptide as depicted in a respective line in column 5 or 8 of Table II, application no. 3, or a homolog or a fragment thereof, a consensus sequence or at least one polypeptide motif as depicted in the respective line in column 8 of Table IV, application no. 3, whereby the respective line discloses in column 7 aspartate; or
(a2) increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, whereby the respective line discloses in column 7 aspartate; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of aspartate or a composition comprising aspartate in said non-human organism or in the culture medium surrounding said non-human organism.

An embodiment of the present invention relates to a process for the production of citrulline, which comprises
(a1) increasing or generating the activity of a polypeptide comprising a polypeptide as depicted in a respective line in column 5 or 8 of Table II, application no. 3, or a homolog or a fragment thereof, a consensus sequence or at least one polypeptide motif as depicted in the respective line in column 8 of Table IV, application no. 3, whereby the respective line discloses in column 7 citrulline; or
(a2) increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, whereby the respective line discloses in column 7 citrulline; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of citrulline or a composition comprising citrulline in said non-human organism or in the culture medium surrounding said non-human organism.

An embodiment of the present invention relates to a process for the production of glycine, which comprises
(a1) increasing or generating the activity of a polypeptide comprising a polypeptide as depicted in a respective line in column 5 or 8 of Table II, application no. 3, or a homolog or a fragment thereof, a consensus sequence or at least one polypeptide motif as depicted in the respective line in column 8 of Table IV, application no. 3, whereby the respective line discloses in column 7 glycine; or
(a2) increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, whereby the respective line discloses in column 7 glycine; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of glycine or a composition comprising glycine in said non-human organism or in the culture medium surrounding said non-human organism.

An embodiment of the present invention relates to a process for the production of homoserine, which comprises
(a1) increasing or generating the activity of a polypeptide comprising a polypeptide as depicted in a respective line in column 5 or 8 of Table II, application no. 3, or a homolog or a fragment thereof, a consensus sequence or at least one polypeptide motif as depicted in the respective line in column 8 of Table IV, application no. 3, whereby the respective line discloses in column 7 homoserine; or
(a2) increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, whereby the respective line discloses in column 7 homoserine; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of homoserine or a composition comprising homoserine in said non-human organism or in the culture medium surrounding said non-human organism.

An embodiment of the present invention relates to a process for the production of ornithine, which comprises
(a1) increasing or generating the activity of a polypeptide comprising a polypeptide as depicted in a respective line in column 5 or 8 of Table II, application no. 3, or a homolog or a fragment thereof, a consensus sequence or at least one polypeptide motif as depicted in the respective line in column 8 of Table IV, application no. 3, whereby the respective line discloses in column 7 ornithine; or
(a2) increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, whereby the respective line discloses in column 7 ornithine; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of ornithine or a composition comprising ornithine in said non-human organism or in the culture medium surrounding said non-human organism.

An embodiment of the present invention relates to a process for the production of phenylalanine, which comprises
(a1) increasing or generating the activity of a polypeptide comprising a polypeptide as depicted in a respective line in column 5 or 8 of Table II, application no. 3, or a homolog or a fragment thereof, a consensus sequence or at least one polypeptide motif as depicted in the respective line in column 8 of Table IV, application no. 3, whereby the respective line discloses in column 7 phenylalanine; or
(a2) increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, whereby the respective line discloses in column 7 phenylalanine; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of phenylalanine or a composition comprising phenylalanine in said non-human organism or in the culture medium surrounding said non-human organism.

An embodiment of the present invention relates to a process for the production of serine, which comprises
(a1) increasing or generating the activity of a polypeptide comprising a polypeptide as depicted in a respective line in column 5 or 8 of Table II, application no. 3, or a homolog or a fragment thereof, a consensus sequence or at least one polypeptide motif as depicted in the respective line in column 8 of Table IV, application no. 3, whereby the respective line discloses in column 7 serine; or
(a2) increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, whereby the respective line discloses in column 7 serine; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of serine or a composition comprising serine in said non-human organism or in the culture medium surrounding said non-human organism.

An embodiment of the present invention relates to a process for the production of tyrosine, which comprises
(a1) increasing or generating the activity of a polypeptide comprising a polypeptide as depicted in a respective line in column 5 or 8 of Table II, application no. 3, or a homolog or a fragment thereof, a consensus sequence or at least one polypeptide motif as depicted in the respective line in column 8 of Table IV, application no. 3, whereby the respective line discloses in column 7 tyrosine; or
(a2) increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, whereby the respective line discloses in column 7 tyrosine; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of tyrosine or a composition comprising tyrosine in said non-human organism or in the culture medium surrounding said non-human organism.

Accordingly, the increase or generation of one or more said activities is for example conferred by one or more expression products of said nucleic acid molecule, e.g. proteins. Accordingly, in the present invention described above, the increase or generation of one or more said activities is for example conferred by one or more protein(s) each comprising a polypeptide selected from the group as depicted in Table II, application no. 3, column 5 and 8, or a homolog or a fragment thereof.

The process of the invention comprises in one embodiment the following steps:
(i) increasing or generating of the expression of; and/or
(ii) increasing or generating the expression of an expression product of; and/or
(iii) increasing or generating one or more activities of an expression product encoded by; at least one nucleic acid molecule (in the following “Fine Chemical Related Protein (FCRP)”-encoding gene or “FCRP”-gene) comprising a nucleic acid molecule selected from the group consisting of:
(a) a nucleic acid molecule encoding the polypeptide shown in column 5 or 8 of Table II, preferably Table II B, application no. 3, or a homolog or a fragment thereof;
(b) a nucleic acid molecule shown in column 5 or 8 of Table I, preferably Table I B, application no. 3, or a homolog or a fragment thereof (preferably the coding region thereof);
(c) a nucleic acid molecule, which, as a result of the degeneracy of the genetic code, can be derived from a polypeptide sequence depicted in column 5 or 8 of Table II, preferably Table II B, application no. 3;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 3, or the coding region thereof;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d),
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 3;
(i) a nucleic acid molecule encoding a polypeptide having the activity represented by a respective protein as depicted in column 5 of Table II, application no. 3;
(j) a nucleic acid molecule which comprises a polynucleotide, which is obtained by amplifying a cDNA library or a genomic library using the primers as depicted in column 8 of Table III, application no. 3; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
   or a nucleic acid molecule comprising a sequence which is complementary thereto.
   (All the references to Tables I to IV are references to the corresponding lines of Tables I to IV.)

In a preferred embodiment thereof said nucleic acid molecule encodes a polypeptide which has the activity of the polypeptide represented by a protein comprising a polypeptide as depicted in the corresponding hit in column 5 of Table II, application no. 3.

In another preferred embodiment thereof said nucleic acid molecule confers a generation or an increase of the respective fine chemical in a non-human organism or a part thereof as compared to a corresponding wild-type non-human organism or part thereof.

In another preferred embodiment thereof said nucleic acid molecule encodes a polypeptide which has the activity of the polypeptide represented by a protein as depicted in the corresponding hit in column 5 of Table II, application no. 3, and confers a generation or an increase of the respective fine chemical in a non-human organism or a part thereof as compared to a corresponding wild-type non-human organism or part thereof.

Accordingly, the genes of the present invention or used in accordance with the present invention, which respectively encode a protein having an activity of 2,3-dihydroxyphenylpropionate 1,2-dioxygenase, 2-C-methyl-D-erythritol 4-phosphate cytidylyltransferase, 2-oxoglutarate dehydrogenase E1 subunit, 30S ribosomal protein S3, 3-deoxy-7-phosphoheptulonate synthase, 3-deoxy-D-arabinoheptulosonate-7-phosphatesynthase, 3oxoacyl-[acyl-carrier-protein] synthase, 3-phosphoglycerate dehydrogenase, 47266012-protein, 49747384_SOYBEAN-protein, 50S ribosomal protein L14, 5′-nucleotidase, ABC metal ion transporter substrate-binding protein, ABC transporter ATP binding component, ABC transporter ATP-binding protein, ABC transporter component, ABC transporter permease protein, ABC transporter substrate-binding protein, acetolactate synthase, acetolactate synthase small subunit, acetyl CoA carboxylase, acetyl-CoA acetyltransferase, acetyltransferase, acid shock protein, acyl transferase, acyl-CoA dehydrogenase, acyl-CoA synthase, adenylate kinase, adenylylsulfate kinase, agmatinase, aldehyde dehydrogenase, amine oxidase, amino acid ABC transporter permease protein, amino acid acetyltransferase, amino acid transporter, amino-acid acetyltransferase, aminotransferase, ammonium transporter, ankyrin repeat family protein, anthranilate synthase component I, arginine exporter protein, arginine N-succinyltransferase, argininosuccinate lyase, asparaginase, aspartate 1-decarboxylase, aspartate aminotransferase, aspartate kinase, aspartate-ammonia ligase, At1g17440-protein, At1g19800-protein, At4g32480-protein, At5g16650-protein, at5g21910-protein, ATP-binding component of a transport system, ATP-dependent RNA helicase, auxin response factor, AX653549-protein, AY087308-protein, b0391-protein, b0456-protein, b0518-protein, b0801-protein, b0841-protein, b0917-protein, b1003-protein, b1024-protein, b1137-protein, b1155-protein, b1163-protein, b1179-protein, b1205-protein, b1229-protein, b1259-protein, b1280-protein, b1330-protein, b1445-protein, b1522-protein, b1583-protein, b1670-protein, b1672-protein, b1837-protein, b1898-protein, b1930-protein, b2032-protein, b2107-protein, b2360-protein, b2399-protein, b2474-protein, b2513-protein, b2613-protein, b2673-protein, b2739-protein, b2812-protein, b2849-protein, b3083-protein, b3098-protein, b3121-protein, b3221-protein, b3246-protein, b3346-protein, b3392-protein, b3410-protein, b3509-protein, b3646-protein, b3791-protein, b3814-protein, b3989-protein, b4029-protein, b4121-protein, beta-galactosidase, betahydroxylase, bifunctional aspartokinase/homoserine dehydrogenase, bifunctional purine biosynthesis protein, branched chain aminotransferase, branched-chain amino acid permease, calcium-dependent protein kinase, carbon dioxide concentrating mechanism protein, cation-transporting ATPase, CBL-interacting protein kinase, CDP-diacylglycerol-glycerol-3-phosphate 3-phosphatidyltransferase, CDP-diacylglycerol-serine 0-phosphatidyltransferase, CDP-glucose-4,6-dehydratase, cell division control protein, cellulose synthase catalytic subunit, Chaperone protein CIpB, chorismate mutase-T and prephenate dehydrogenase, citrate synthase, coproporphyrinogen Ill oxidase, CTP synthetase, cullin, cyclin D, cystathionine gammasynthase, cytochrome c-type protein, D-3-phosphoglycerate dehydrogenase, delta-1-pyrroline 5-carboxylase synthetase, diacylglycerol O-acyltransferase, dihydroxyacid dehydratase, DNA helicase II, DNA mismatch repair protein, DNA-binding protein, DnaJ-like chaperone, D-serine dehydratase, electron transfer flavoprotein subunit beta, elongation factor Tu, enoyl-CoA hydratase, enterobacterial common antigen polymerase, eukaryotic translation initiation factor, eukaryotic translation initiation factor 5, exopolyphosphatase, Fe—S subunit of oxidoreductase, flavodoxin, fumarylacetoacetate hydrolase, gamma-glutamyltranspeptidase, GDP-mannose dehydrogenase, geranylgeranyl pyrophosphate synthase, gibberellin 20-oxidase, Glu/Leu/Phe/Val dehydrogenase , glucokinase, glucose dehydrogenase, glucose-1-phosphate cytidylyltransferase, glucose-6-phosphate 1-dehydrogenase, glutamate-ammonia-ligase, glutamine amidotransferase, glutamine synthetase, glutaredoxin, glutathione peroxidase, glutathione S-transferase, Glycine cleavage system T aminomethyltransferase, glycogen (starch) synthase, glycogen synthase, glycoprotease, glycosyl transferase, GM02LC11114-protein, GM02LC17485-protein, harpin-induced family protein, heat shock transcription factor, HesB/YadR/YfhF family protein, histone acetyltransferase, histone H2A, homocitrate synthase, homoserine O-acetyltransferase, hydrolase, integral membrane transporter family protein, iron(III) dicitrate-binding protein, isochorismate synthase, kinase, L-aspartate oxidase, lipoprotein precursor, lysine decarboxylase, lysyl-tRNA synthetase, major facilitator superfamily transporter protein, malate dehydrogenase, malic enzyme, membrane protein, membrane transport protein, metal ion transporter, metal-dependent hydrolase, methylglyoxal synthase, methylglyoxalase, methyltransferase, mitochondrial inner membrane protease subunit, molecular chaperone portein, monothiol glutaredoxin, monthiol glutaredoxin, multidrug resistance protein, multiple antibiotic resistance protein, murein transglycosylase, N-(5′-phosphoribosyl)anthranilate isomerase, Na+/K+ transporter, N-acetyl-gamma-glutamyl-phosphate reductase, NADH dehydrogenase I chain I, NADH dehydrogenase subunit N, NADH-quinone oxidoreductase subunit, NADP-dependent malic enzyme, nitrate/nitrite transport protein, O-antigen chain length determinant, oxidoreductase, oxidoreductase subunit, oxireductase, peptidase, permease protein of phosphate ABC transporter, phosphate permease, phosphate transporter, phosphoadenosine phosphosulfate reductase , phosphoanhydride phosphorylase, phosphoenolpyruvate carboxykinase, phosphopantetheine adenylyltransferase, phosphoribosylformyl glycinamidine synthase subunit, Photosystem 1 reaction center subunit XI, photosystem II protein, polygalacturonase, precorrin methylase, Pre-mRNA-splicing factor, prolipoprotein diacylglyceryl transferase, protease, protein kinase, protein phosphatase, PTS system N-acetylmuramic acid-specific EllBC component, purine biosynthesis protein, pyruvate kinase, restriction alleviation protein, riboflavin biosynthesis protein, RNA binding protein, RNA polymerase sigma factor, RNA-binding protein, s_pp015018333r-protein, sec-independent protein translocase, Sec-independent protein translocase subunit, serine acetyltransferase, serine hydroxymethyltransferase, serine protease, serine/threonine dehydratase, Serine/threonine-protein phosphatase, short-chain alcohol dehydrogenase family, sll0281-protein, sill 032-protein, sill 761-protein, sn-glycerol-3-phosphate dehydrogenase, sodium/proton antiporter, sterol 24-C-methyltransferase, thioredoxin, thioredoxin family protein, threonine dehydratase, threonine synthase, transcription factor, transcriptional regulator, transport protein, trehalose-phosphatase, tryptophan biosynthesis protein, tryptophan synthase alpha chain, TTC0768-protein, TTC0881-protein, TTC1386-protein, urease subunit, uridine/cytidine kinase, uroporphyrin-III C-methyltransferase, XM_{—}473199-protein, ydr183w-protein, ydr273w-protein, ydr507c-protein, yer014w-protein, yer106w-protein, YFLO19C-protein, yfl054c-protein, ygl096w-protein, ygl237c-protein, ygr221c-protein, yhl013c-protein, yhr006w-protein, yhr207c-protein, YKL038W-protein, yml083c-protein, ymr013c-protein, YNL086W-protein, yol160w-protein, zinc finger protein, zinc transporter, or Zm_{—}4842_BE510522-protein, which respectively encode a protein comprising a polypeptide encoded by a nucleic acid sequence as shown in Table I, application no. 3, column 5 or 8, (preferably the coding region thereof), or a homolog or a fragment thereof, which respectively encode a protein comprising a polypeptide as depicted in Table II, application no. 3, column 5 or 8, or a homolg or a fragment thereof, and/or which respectively can be amplified with the primer set shown in Table III, application no. 3, column 8, are also referred to as “FCRP genes”.

Proteins or polypeptides encoded by “FCRP-genes” are referred to as “Fine Chemical Related Proteins” or “FCRP”. For the purposes of the description of the present invention, the respective protein having an activity of 2,3-dihydroxyphenylpropionate 1,2-dioxygenase, 2-C-methyl-D-erythritol 4-phosphate cytidylyltransferase, 2-oxoglutarate dehydrogenase E1 subunit, 30S ribosomal protein S3, 3-deoxy-7-phosphoheptulonate synthase, 3-deoxy-D-arabinoheptulosonate-7-phosphatesynthase, 3-oxoacyl-[acyl-carrier-protein] synthase, 3-phosphoglycerate dehydrogenase, 47266012-protein, 49747384_SOYBEAN-protein, 50S ribosomal protein L14, 5′-nucleotidase, ABC metal ion transporter substrate-binding protein, ABC transporter ATP binding component, ABC transporter ATP-binding protein, ABC transporter component, ABC transporter permease protein, ABC transporter substrate-binding protein, acetolactate synthase, acetolactate synthase small subunit, acetyl CoA carboxylase, acetyl-CoA acetyltransferase, acetyltransferase, acid shock protein, acyl transferase, acyl-CoA dehydrogenase, acyl-CoA synthase, adenylate kinase, adenylylsulfate kinase, agmatinase, aldehyde dehydrogenase, amine oxidase, amino acid ABC transporter permease protein, amino acid acetyltransferase, amino acid transporter, amino-acid acetyltransferase, aminotransferase, ammonium transporter, ankyrin repeat family protein, anthranilate synthase component I, arginine exporter protein, arginine N-succinyltransferase, argininosuccinate lyase, asparaginase, aspartate 1-decarboxylase, aspartate aminotransferase, aspartate kinase, aspartate-ammonia ligase, At1g17440-protein, At1g19800-protein, At4g32480-protein, At5g16650-protein, at5g21910-protein, ATP-binding component of a transport system, ATP-dependent RNA helicase, auxin response factor, AX653549-protein, AY087308-protein, b0391-protein, b0456protein, b0518-protein, b0801-protein, b0841-protein, b0917-protein, b1003-protein, b1024-protein, b1137-protein, b1155-protein, b1163-protein, b1179-protein, b1205-protein, b1229-protein, b1259-protein, b1280-protein, b1330-protein, b1445-protein, b1522-protein, b1583-protein, b1670-protein, b1672-protein, b1837-protein, b1898-protein, b1930-protein, b2032-protein, b2107-protein, b2360-protein, b2399-protein, b2474-protein, b2513-protein, b2613-protein, b2673-protein, b2739-protein, b2812-protein, b2849-protein, b3083-protein, b3098-protein, b3121-protein, b3221-protein, b3246-protein, b3346-protein, b3392-protein, b3410-protein, b3509-protein, b3646-protein, b3791-protein, b3814-protein, b3989-protein, b4029-protein, b4121-protein, beta-galactosidase, beta-hydroxylase, bifunctional aspartokinase/homoserine dehydrogenase, bifunctional purine biosynthesis protein, branched chain aminotransferase, branched-chain amino acid permease, calcium-dependent protein kinase, carbon dioxide concentrating mechanism protein, cation-transporting ATPase, CBL-interacting protein kinase, CDP-diacylglycerol-glycerol-3-phosphate 3-phosphatidyltransferase, CDP-diacylglycerol-serine O-phosphatidyltransferase, CDP-glucose-4,6-dehydratase, cell division control protein, cellulose synthase catalytic subunit, Chaperone protein CIpB, chorismate mutase-T and prephenate dehydrogenase, citrate synthase, coproporphyrinogen III oxidase, CTP synthetase, cullin, cyclin D, cystathionine gamma-synthase, cytochrome c-type protein, D-3-phosphoglycerate dehydrogenase, delta-1-pyrroline 5-carboxylase synthetase, diacylglycerol O-acyltransferase, dihydroxyacid dehydratase, DNA helicase II, DNA mismatch repair protein, DNA-binding protein, DnaJ-like chaperone, D-serine dehydratase, electron transfer flavoprotein subunit beta, elongation factor Tu, enoyl-CoA hydratase, enterobacterial common antigen polymerase, eukaryotic translation initiation factor, eukaryotic translation initiation factor 5, exopolyphosphatase, Fe—S subunit of oxidoreductase, flavodoxin, fumarylacetoacetate hydrolase, gamma-glutamyltranspeptidase, GDP-mannose dehydrogenase, geranylgeranyl pyrophosphate synthase, gibberellin 20-oxidase, Glu/Leu/Phe/Val dehydrogenase , glucokinase, glucose dehydrogenase, glucose-1-phosphate cytidylyltransferase, glucose-6-phosphate 1-dehydrogenase, glutamate-ammonia-ligase, glutamine amidotransferase, glutamine synthetase, glutaredoxin, glutathione peroxidase, glutathione S-transferase, Glycine cleavage system T aminomethyltransferase, glycogen (starch) synthase, glycogen synthase, glycoprotease, glycosyl transferase, GM02LC11114-protein, GM02LC17485-protein, harpin-induced family protein, heat shock transcription factor, HesB/YadR/YfhF family protein, histone acetyltransferase, histone H2A, homocitrate synthase, homoserine O-acetyltransferase, hydrolase, integral membrane transporter family protein, iron(III) dicitrate-binding protein, isochorismate synthase, kinase, L-aspartate oxidase, lipoprotein precursor, lysine decarboxylase, lysyl-tRNA synthetase, major facilitator superfamily transporter protein, malate dehydrogenase, malic enzyme, membrane protein, membrane transport protein, metal ion transporter, metal-dependent hydrolase, methylglyoxal synthase, methylglyoxalase, methyltransferase, mitochondrial inner membrane protease subunit, molecular chaperone portein, monothiol glutaredoxin, monthiol glutaredoxin, multidrug resistance protein, multiple antibiotic resistance protein, murein transglycosylase, N-(5′-phosphoribosyl)anthranilate isomerase, Na+/K+ transporter, N-acetyl-gamma-glutamylphosphate reductase, NADH dehydrogenase I chain I, NADH dehydrogenase subunit N, NADH-quinone oxidoreductase subunit, NADP-dependent malic enzyme, nitrate/nitrite transport protein, O-antigen chain length determinant, oxidoreductase, oxidoreductase subunit, oxireductase, peptidase, permease protein of phosphate ABC transporter, phosphate permease, phosphate transporter, phosphoadenosine phosphosulfate reductase , phosphoanhydride phosphorylase, phosphoenolpyruvate carboxykinase, phosphopantetheine adenylyltransferase, phosphoribosylformyl glycinamidine synthase subunit, Photosystem I reaction center subunit XI, photosystem II protein, polygalacturonase, precorrin methylase, Pre-mRNA-splicing factor, prolipoprotein diacylglyceryl transferase, protease, protein kinase, protein phosphatase, PTS system N-acetylmuramic acid-specific EIIBC component, purine biosynthesis protein, pyruvate kinase, restriction alleviation protein, riboflavin biosynthesis protein, RNA binding protein, RNA polymerase sigma factor, RNA-binding protein, s_pp015018333r-protein, sec-independent protein translocase, Sec-independent protein translocase subunit, serine acetyltransferase, serine hydroxymethyltransferase, serine protease, serine/threonine dehydratase, Serine/threonineprotein phosphatase, short-chain alcohol dehydrogenase family, sll0281-protein, sll1032-protein, sll1761-protein, sn-glycerol-3-phosphate dehydrogenase, sodium/proton antiporter, sterol 24-C-methyltransferase, thioredoxin, thioredoxin family protein, threonine dehydratase, threonine synthase, transcription factor, transcriptional regulator, transport protein, trehalosephosphatase, tryptophan biosynthesis protein, tryptophan synthase alpha chain, TTC0768protein, TTC0881-protein, TTC1386-protein, urease subunit, uridine/cytidine kinase, uroporphyrin-III C-methyltransferase, XM_{—}473199-protein, ydr183w-protein, ydr273w-protein, ydr507c-protein, yer014w-protein, yer106w-protein, YFL019C-protein, yfl054c-protein, ygl096w-protein, ygl237c-protein, ygr221 c-protein, yhl013c-protein, yhr006w-protein, yhr207c-protein, YKL038W-protein, yml083c-protein, ymr013c-protein, YNL086W-protein, yol160w-protein, zinc finger protein, zinc transporter, or Zm_{—}4842_BE510522-protein, the respective protein comprising a polypeptide encoded by one or more respective nucleic acid sequences as shown in Table I, application no. 3, column 5 or 8, (preferably the coding region thereof), or a homolog or fragment thereof, the respective protein comprising a respective polypeptide as depicted in Table II, application no. 3, column 5 or 8, or a homolog or fragment thereof, the respective protein comprising a sequence corresponding to the consensus sequence as shown in Table IV, application no. 3, column 8, and/or the respective protein comprising at least one polypeptide motif as shown in Table IV, application no. 3, column 8 is also referred to as Fine Chemical Related Protein” or “FCRP”.

Thus, in one embodiment, the present invention provides a process of the production of 3,4-dihydroxyphenylalanine (dopa), 5-oxoproline, alanine, asparagine, aspartate, citrulline, glycine, homoserine, ornithine, phenylalanine, serine, and/or tyrosine, by increasing or generating one or more activities, especially selected from the group consisting of 2,3-dihydroxyphenylpropionate 1,2-dioxygenase, 2-C-methyl-D-erythritol 4-phosphate cytidylyltransferase, 2-oxoglutarate dehydrogenase E1 subunit, 30S ribosomal protein S3, 3-deoxy-7-phosphoheptulonate synthase, 3-deoxy-D-arabinoheptulosonate-7-phosphatesynthase, 3-oxoacyl-[acyl-carrier-protein] synthase, 3-phosphoglycerate dehydrogenase, 47266012-protein, 49747384_SOYBEAN-protein, 50S ribosomal protein L14, 5′-nucleotidase, ABC metal ion transporter substrate-binding protein, ABC transporter ATP binding component, ABC transporter ATP-binding protein, ABC transporter component, ABC transporter permease protein, ABC transporter substrate-binding protein, acetolactate synthase, acetolactate synthase small subunit, acetyl CoA carboxylase, acetyl-CoA acetyltransferase, acetyltransferase, acid shock protein, acyl transferase, acyl-CoA dehydrogenase, acyl-CoA synthase, adenylate kinase, adenylylsulfate kinase, agmatinase, aldehyde dehydrogenase, amine oxidase, amino acid ABC transporter permease protein, amino acid acetyltransferase, amino acid transporter, amino-acid acetyltransferase, aminotransferase, ammonium transporter, ankyrin repeat family protein, anthranilate synthase component I, arginine exporter protein, arginine N-succinyltransferase, argininosuccinate lyase, asparaginase, aspartate 1-decarboxylase, aspartate aminotransferase, aspartate kinase, aspartate-ammonia ligase, At1g17440-protein, At1g19800-protein, At4g32480-protein, At5g16650-protein, at5g21910-protein, ATP-binding component of a transport system, ATP-dependent RNA helicase, auxin response factor, AX653549-protein, AY087308-protein, b0391-protein, b0456-protein, b0518-protein, b0801-protein, b0841-protein, b0917-protein, b1003-protein, b1024-protein, b1137-protein, b1155-protein, b1163-protein, b1179-protein, b1205-protein, b1229-protein, b1259-protein, b1280-protein, b1330-protein, b1445-protein, b1522-protein, b1583-protein, b1670-protein, b1672-protein, b1837-protein, b1898-protein, b1930-protein, b2032-protein, b2107-protein, b2360-protein, b2399-protein, b2474-protein, b2513-protein, b2613-protein, b2673-protein, b2739-protein, b2812-protein, b2849-protein, b3083-protein, b3098-protein, b3121-protein, b3221-protein, b3246-protein, b3346-protein, b3392-protein, b3410-protein, b3509-protein, b3646-protein, b3791-protein, b3814-protein, b3989-protein, b4029-protein, b4121-protein, beta-galactosidase, betahydroxylase, bifunctional aspartokinase/homoserine dehydrogenase, bifunctional purine biosynthesis protein, branched chain aminotransferase, branched-chain amino acid permease, calcium-dependent protein kinase, carbon dioxide concentrating mechanism protein, cation-transporting ATPase, CBL-interacting protein kinase, CDP-diacylglycerol-glycerol-3-phosphate 3-phosphatidyltransferase, CDP-diacylglycerol-serine O-phosphatidyltransferase, CDP-glucose-4,6-dehydratase, cell division control protein, cellulose synthase catalytic subunit, Chaperone protein CIpB, chorismate mutase-T and prephenate dehydrogenase, citrate synthase, coproporphyrinogen III oxidase, CTP synthetase, cullin, cyclin D, cystathionine gammasynthase, cytochrome c-type protein, D-3-phosphoglycerate dehydrogenase, delta-1-pyrroline 5-carboxylase synthetase, diacylglycerol O-acyltransferase, dihydroxyacid dehydratase, DNA helicase II, DNA mismatch repair protein, DNA-binding protein, DnaJ-like chaperone, D-serine dehydratase, electron transfer flavoprotein subunit beta, elongation factor Tu, enoyl-CoA hydratase, enterobacterial common antigen polymerase, eukaryotic translation initiation factor, eukaryotic translation initiation factor 5, exopolyphosphatase, Fe—S subunit of oxidoreductase, flavodoxin, fumarylacetoacetate hydrolase, gamma-glutamyltranspeptidase, GDP-mannose dehydrogenase, geranylgeranyl pyrophosphate synthase, gibberellin 20-oxidase, Glu/Leu/Phe/Val dehydrogenase , glucokinase, glucose dehydrogenase, glucose-1-phosphate cytidylyltransferase, glucose-6-phosphate 1-dehydrogenase, glutamate-ammonia-ligase, glutamine amidotransferase, glutamine synthetase, glutaredoxin, glutathione peroxidase, glutathione S-transferase, Glycine cleavage system T aminomethyltransferase, glycogen (starch) synthase, glycogen synthase, glycoprotease, glycosyl transferase, GM02LC11114-protein, GM02LC17485-protein, harpin-induced family protein, heat shock transcription factor, HesB/YadR/YfhF family protein, histone acetyltransferase, histone H2A, homocitrate synthase, homoserine O-acetyltransferase, hydrolase, integral membrane transporter family protein, iron(III) dicitrate-binding protein, isochorismate synthase, kinase, L-aspartate oxidase, lipoprotein precursor, lysine decarboxylase, lysyl-tRNA synthetase, major facilitator superfamily transporter protein, malate dehydrogenase, malic enzyme, membrane protein, membrane transport protein, metal ion transporter, metal-dependent hydrolase, methylglyoxal synthase, methylglyoxalase, methyltransferase, mitochondrial inner membrane protease subunit, molecular chaperone portein, monothiol glutaredoxin, monthiol glutaredoxin, multidrug resistance protein, multiple antibiotic resistance protein, murein transglycosylase, N-(5′-phosphoribosyl)anthranilate isomerase, Na+/K+ transporter, N-acetyl-gamma-glutamyl-phosphate reductase, NADH dehydrogenase I chain I, NADH dehydrogenase subunit N, NADH-quinone oxidoreductase subunit, NADP-dependent malic enzyme, nitrate/nitrite transport protein, O-antigen chain length determinant, oxidoreductase, oxidoreductase subunit, oxireductase, peptidase, permease protein of phosphate ABC transporter, phosphate permease, phosphate transporter, phosphoadenosine phosphosulfate reductase , phosphoanhydride phosphorylase, phosphoenolpyruvate carboxykinase, phosphopantetheine adenylyltransferase, phosphoribosylformyl glycinamidine synthase subunit, Photosystem I reaction center subunit XI, photosystem II protein, polygalacturonase, precorrin methylase, Pre-mRNA-splicing factor, prolipoprotein diacylglyceryl transferase, protease, protein kinase, protein phosphatase, PTS system N-acetylmuramic acid-specific Ell BC component, purine biosynthesis protein, pyruvate kinase, restriction alleviation protein, riboflavin biosynthesis protein, RNA binding protein, RNA polymerase sigma factor, RNA-binding protein, s_pp015018333r-protein, sec-independent protein translocase, Sec-independent protein translocase subunit, serine acetyltransferase, serine hydroxymethyltransferase, serine protease, serine/threonine dehydratase, Serine/threonine-protein phosphatase, short-chain alcohol dehydrogenase family, sll0281-protein, sill 032-protein, sill 761-protein, sn-glycerol-3-phosphate dehydrogenase, sodium/proton antiporter, sterol 24-C-methyltransferase, thioredoxin, thioredoxin family protein, threonine dehydratase, threonine synthase, transcription factor, transcriptional regulator, transport protein, trehalose-phosphatase, tryptophan biosynthesis protein, tryptophan synthase alpha chain, TTC0768-protein, TTC0881-protein, TTC1386-protein, urease subunit, uridine/cytidine kinase, uroporphyrin-III C-methyltransferase, XM_{—}473199-protein, ydr183w-protein, ydr273w-protein, ydr507c-protein, yer014w-protein, yer106w-protein, YFLO19C-protein, yfl054c-protein, ygl096w-protein, ygl237c-protein, ygr221c-protein, yhl013c-protein, yhr006w-protein, yhr207c-protein, YKL038W-protein, yml083c-protein, ymr013c-protein, YNL086W-protein, yol160w-protein, zinc finger protein, zinc transporter, and Zm_{—}4842_BE510522-protein, which is conferred by one or more FCRPs or the gene product of one or more FCRP-genes, for example by the gene product of a nucleic acid sequences comprising a polynucleotide selected from the group as shown in Table I, application no. 3, column 5 or 8, (preferably by the coding region thereof), or a homolog or a fragment thereof, e.g. or by one or more proteins each comprising a polypeptide encoded by one or more nucleic acid sequences selected from the group as shown in Table I, application no. 3, column 5 or 8, (preferably by the coding region thereof), or a homolog or a fragment thereof, or by one or more protein(s) each comprising a polypeptide selected from the group as depicted in Table II, application no. 3, column 5 and 8, or a homolog thereof, or a protein comprising a sequence corresponding to the consensus sequence or comprising at least one polypeptide motif as shown in Table IV, application no. 3, column 8.

for the disclosure of this paragraph see [0025.1.1.1] above.

In an embodiment, the process comprises increasing or generating the activity of one or more polypeptides having said activity, e.g. by generating or increasing the amount and/or specific activity in the cell or a compartment of a cell of one of more FCRP, especially selected from the group consisting of 2,3-dihydroxyphenylpropionate 1,2-dioxygenase, 2-C-methyl-D-erythritol 4-phosphate cytidylyltransferase, 2-oxoglutarate dehydrogenase E1 subunit, 30S ribosomal protein S3, 3-deoxy-7-phosphoheptulonate synthase, 3-deoxy-D-arabinoheptulosonate-7-phosphatesynthase, 3-oxoacyl-[acyl-carrier-protein] synthase, 3-phosphoglycerate dehydrogenase, 47266012-protein, 49747384_SOYBEAN-protein, 50S ribosomal protein L14, 5′-nucleotidase, ABC metal ion transporter substrate-binding protein, ABC transporter ATP binding component, ABC transporter ATP-binding protein, ABC transporter component, ABC transporter permease protein, ABC transporter substrate-binding protein, acetolactate synthase, acetolactate synthase small subunit, acetyl CoA carboxylase, acetyl-CoA acetyltransferase, acetyltransferase, acid shock protein, acyl transferase, acyl-CoA dehydrogenase, acyl-CoA synthase, adenylate kinase, adenylylsulfate kinase, agmatinase, aldehyde dehydrogenase, amine oxidase, amino acid ABC transporter permease protein, amino acid acetyltransferase, amino acid transporter, amino-acid acetyltransferase, aminotransferase, ammonium transporter, ankyrin repeat family protein, anthranilate synthase component I, arginine exporter protein, arginine N-succinyltransferase, argininosuccinate lyase, asparaginase, aspartate 1-decarboxylase, aspartate aminotransferase, aspartate kinase, aspartate-ammonia ligase, At1g17440-protein, At1g19800-protein, At4g32480-protein, At5g16650-protein, at5g21910-protein, ATP-binding component of a transport system, ATP-dependent RNA helicase, auxin response factor, AX653549-protein, AY087308-protein, b0391-protein, b0456-protein, b0518-protein, b0801-protein, b0841-protein, b0917-protein, b1003-protein, b1024-protein, b1137-protein, b1155-protein, b1163-protein, b1179-protein, b1205-protein, b1229-protein, b1259-protein, b1280-protein, b1330-protein, b1445-protein, b1522-protein, b1583-protein, b1670-protein, b1672-protein, b1837-protein, b1898-protein, b1930-protein, b2032-protein, b2107-protein, b2360-protein, b2399-protein, b2474-protein, b2513-protein, b2613-protein, b2673-protein, b2739-protein, b2812-protein, b2849-protein, b3083-protein, b3098-protein, b3121-protein, b3221-protein, b3246-protein, b3346-protein, b3392-protein, b3410-protein, b3509-protein, b3646-protein, b3791-protein, b3814-protein, b3989-protein, b4029-protein, b4121-protein, beta-galactosidase, beta-hydroxylase, bifunctional aspartokinase/homoserine dehydrogenase, bifunctional purine biosynthesis protein, branched chain aminotransferase, branched-chain amino acid permease, calcium-dependent protein kinase, carbon dioxide concentrating mechanism protein, cation-transporting ATPase, CBL-interacting protein kinase, CDP-diacylglycerol-glycerol-3-phosphate 3-phosphatidyltransferase, CDP-diacylglycerol-serine O-phosphatidyltransferase, CDP-glucose-4,6-dehydratase, cell division control protein, cellulose synthase catalytic subunit, Chaperone protein CIpB, chorismate mutase-T and prephenate dehydrogenase, citrate synthase, coproporphyrinogen III oxidase, CTP synthetase, cullin, cyclin D, cystathionine gammasynthase, cytochrome c-type protein, D-3-phosphoglycerate dehydrogenase, delta-1-pyrroline 5-carboxylase synthetase, diacylglycerol O-acyltransferase, dihydroxyacid dehydratase, DNA helicase II, DNA mismatch repair protein, DNA-binding protein, DnaJ-like chaperone, D-serine dehydratase, electron transfer flavoprotein subunit beta, elongation factor Tu, enoyl-CoA hydratase, enterobacterial common antigen polymerase, eukaryotic translation initiation factor, eukaryotic translation initiation factor 5, exopolyphosphatase, Fe—S subunit of oxidoreductase, flavodoxin, fumarylacetoacetate hydrolase, gamma-glutamyltranspeptidase, GDP-mannose dehydrogenase, geranylgeranyl pyrophosphate synthase, gibberellin 20-oxidase, Glu/Leu/Phe/Val dehydrogenase , glucokinase, glucose dehydrogenase, glucose-1-phosphate cytidylyltransferase, glucose-6-phosphate 1-dehydrogenase, glutamate-ammonia-ligase, glutamine amidotransferase, glutamine synthetase, glutaredoxin, glutathione peroxidase, glutathione S-transferase, Glycine cleavage system T aminomethyltransferase, glycogen (starch) synthase, glycogen synthase, glycoprotease, glycosyl transferase, GM02LC11114-protein, GM02LC17485-protein, harpin-induced family protein, heat shock transcription factor, HesB/YadR/YfhF family protein, histone acetyltransferase, histone H2A, homocitrate synthase, homoserine O-acetyltransferase, hydrolase, integral membrane transporter family protein, iron(III) dicitrate-binding protein, isochorismate synthase, kinase, L-aspartate oxidase, lipoprotein precursor, lysine decarboxylase, lysyl-tRNA synthetase, major facilitator superfamily transporter protein, malate dehydrogenase, malic enzyme, membrane protein, membrane transport protein, metal ion transporter, metal-dependent hydrolase, methylglyoxal synthase, methylglyoxalase, methyltransferase, mitochondrial inner membrane protease subunit, molecular chaperone portein, monothiol glutaredoxin, monthiol glutaredoxin, multidrug resistance protein, multiple antibiotic resistance protein, murein transglycosylase, N-(5′-phosphoribosyl)anthranilate isomerase, Na+/K+ transporter, N-acetyl-gamma-glutamyl-phosphate reductase, NADH dehydrogenase I chain I, NADH dehydrogenase subunit N, NADH-quinone oxidoreductase subunit, NADP-dependent malic enzyme, nitrate/nitrite transport protein, O-antigen chain length determinant, oxidoreductase, oxidoreductase subunit, oxireductase, peptidase, permease protein of phosphate ABC transporter, phosphate permease, phosphate transporter, phosphoadenosine phosphosulfate reductase , phosphoanhydride phosphorylase, phosphoenolpyruvate carboxykinase, phosphopantetheine adenylyltransferase, phosphoribosylformyl glycinamidine synthase subunit, Photosystem I reaction center subunit XI, photosystem II protein, polygalacturonase, precorrin methylase, Pre-mRNA-splicing factor, prolipoprotein diacylglyceryl transferase, protease, protein kinase, protein phosphatase, PTS system N-acetylmuramic acid-specific Ell BC component, purine biosynthesis protein, pyruvate kinase, restriction alleviation protein, riboflavin biosynthesis protein, RNA binding protein, RNA polymerase sigma factor, RNA-binding protein, s_pp015018333r-protein, sec-independent protein translocase, Sec-independent protein translocase subunit, serine acetyltransferase, serine hydroxymethyltransferase, serine protease, serine/threonine dehydratase, Serine/threonine-protein phosphatase, short-chain alcohol dehydrogenase family, sll0281-protein, sll1032-protein, sll1761-protein, sn-glycerol-3-phosphate dehydrogenase, sodium/proton antiporter, sterol 24-C-methyltransferase, thioredoxin, thioredoxin family protein, threonine dehydratase, threonine synthase, transcription factor, transcriptional regulator, transport protein, trehalose-phosphatase, tryptophan biosynthesis protein, tryptophan synthase alpha chain, TTC0768-protein, TTC0881-protein, TTC1386protein, urease subunit, uridine/cytidine kinase, uroporphyrin-111C-methyltransferase, XM_{—}473199-protein, ydr183w-protein, ydr273w-protein, ydr507c-protein, yer014w-protein, yer106w-protein, YFLO19C-protein, yfl054c-protein, ygl096w-protein, ygl237c-protein, ygr221 cprotein, yhl013c-protein, yhr006w-protein, yhr207c-protein, YKL038W-protein, yml083c-protein, ymr013c-protein, YNL086W-protein, yol160w-protein, zinc finger protein, zinc transporter, and Zm_{—}4842_BE510522-protein, for example of the respective polypeptide as depicted in Table II, application no. 3, column 5 and 8, or a homolog or a fragment thereof, or the respective polypeptide comprising a sequence corresponding to the consensus sequences as shown in Table IV, application no. 3, column 8, or the respective polypeptide comprising at least one polypeptide motif as depicted in Table IV, application no. 3, column 8.

A further embodiment of the present invention relates to a process for the production of aspartate, which comprises
(a) increasing or generating the activity of a 47266012-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of tyrosine, which comprises
(a) increasing or generating the activity of a 47266012-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a 47266012-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of aspartate, which comprises
(a) increasing or generating the activity of a 49747384_SOYBEAN-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of tyrosine, which comprises
(a) increasing or generating the activity of a 49747384_SOYBEAN-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a 49747384_SOYBEAN-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of alanine, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of citrulline, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of asparagine, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of serine, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of alanine, which comprises
(a) increasing or generating the activity of a glucose-6-phosphate 1-dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating the activity of a glucose-6-phosphate 1-dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a glucose-6-phosphate 1-dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating the activity of a amino acid acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of citrulline, which comprises
(a) increasing or generating the activity of a amino acid acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of phenylalanine, which comprises
(a) increasing or generating the activity of a amino acid acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of citrulline, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of glycine, which comprises
(a) increasing or generating the activity of a DNA-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of serine, which comprises
(a) increasing or generating the activity of a DNA-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of phenylalanine, which comprises
(a) increasing or generating the activity of a At1g17440-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a At1g17440-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating the activity of a At1g19800-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of citrulline, which comprises
(a) increasing or generating the activity of a At1g19800-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of tyrosine, which comprises
(a) increasing or generating the activity of a At1g19800-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating the activity of a cullin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a cullin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of homoserine, which comprises
(a) increasing or generating the activity of a bifunctional aspartokinase/homoserine dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

A further embodiment of the present invention relates to a process for the production of aspartate, which comprises
(a) increasing or generating the activity of a eukaryotic translation initiation factor 5 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a eukaryotic translation initiation factor 5 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a eukaryotic translation initiation factor 5 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a eukaryotic translation initiation factor 5 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a eukaryotic translation initiation factor 5 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of phenylalanine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of glycine, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of alanine, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of glycine, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of tyrosine, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of alanine, which comprises
(a) increasing or generating the activity of a aminotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of 5-oxoproline, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of asparagine, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of phenylalanine, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating the activity of a CBL-interacting protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a CBL-interacting protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a CBL-interacting protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of serine, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of glycine, which comprises
(a) increasing or generating the activity of a CBL-interacting protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of alanine, which comprises
(a) increasing or generating the activity of a delta-1-pyrroline 5-carboxylase synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of glycine, which comprises
(a) increasing or generating the activity of a delta-1-pyrroline 5-carboxylase synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of tyrosine, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating the activity of a ankyrin repeat family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating the activity of a thioredoxin family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a thioredoxin family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a thioredoxin family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating the activity of a harpin-induced family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a harpin-induced family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a harpin-induced family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating the activity of a DNA mismatch repair protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of phenylalanine, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of serine, which comprises
(a) increasing or generating the activity of a eukaryotic translation initiation factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a glucose-6-phosphate 1-dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating the activity of a glycosyl transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a glycosyl transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating the activity of a auxin response factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a auxin response factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating the activity of a monthiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a monthiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of phenylalanine, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of phenylalanine, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of tyrosine, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of tyrosine, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of serine, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of alanine, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of aspartate, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of citrulline, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of alanine, which comprises
(a) increasing or generating the activity of a At4g32480-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of asparagine, which comprises
(a) increasing or generating the activity of a At4g32480-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of glycine, which comprises
(a) increasing or generating the activity of a At4g32480-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating the activity of a At4g32480-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a At4g32480-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a At4g32480-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of citrulline, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of phenylalanine, which comprises
(a) increasing or generating the activity of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of asparagine, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of phenylalanine, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of tyrosine, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of 3,4-dihydroxyphenylalanine (dopa), which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4-dihydroxyphenylalanine (dopa) or a composition comprising 3,4-dihydroxyphenylalanine (dopa) in said non-human organism or in the culture medium surrounding said non-human organism.

A further embodiment of the present invention relates to a process for the production of phenylalanine, which comprises
(a) increasing or generating the activity of a gibberellin 20-oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a gibberellin 20-oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of serine, which comprises
(a) increasing or generating the activity of a integral membrane transporter family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of phenylalanine, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of asparagine, which comprises
(a) increasing or generating the activity of a At5g16650-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of aspartate, which comprises
(a) increasing or generating the activity of a At5g16650-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of serine, which comprises
(a) increasing or generating the activity of a At5g16650-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of 5-oxoproline, which comprises
(a) increasing or generating the activity of a At5g16650-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of phenylalanine, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating the activity of a at5g21910-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of serine, which comprises
(a) increasing or generating the activity of a thioredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of alanine, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of citrulline, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of citrulline, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of phenylalanine, which comprises
(a) increasing or generating the activity of a RNA-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a RNA-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of alanine, which comprises
(a) increasing or generating the activity of a zinc finger protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating the activity of a zinc finger protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a zinc finger protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of 5-oxoproline, which comprises
(a) increasing or generating the activity of a zinc finger protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of aspartate, which comprises
(a) increasing or generating the activity of a CDP-diacylglycerol-serine O-phosphatidyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of 5-oxoproline, which comprises
(a) increasing or generating the activity of a CDP-diacylglycerol-serine O-phosphatidyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of serine, which comprises
(a) increasing or generating the activity of a Glycine cleavage system T aminomethyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of serine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating the activity of a adenylylsulfate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of alanine, which comprises
(a) increasing or generating the activity of a malic enzyme non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating the activity of a malic enzyme non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a malic enzyme non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of alanine, which comprises
(a) increasing or generating the activity of a sec-independent protein translocase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of alanine, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of alanine, which comprises
(a) increasing or generating the activity of a beta-hydroxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of aspartate, which comprises
(a) increasing or generating the activity of a beta-hydroxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of serine, which comprises
(a) increasing or generating the activity of a beta-hydroxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating the activity of a elongation factor Tu non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a elongation factor Tu non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a elongation factor Tu non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a elongation factor Tu non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a elongation factor Tu non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of tyrosine, which comprises
(a) increasing or generating the activity of a 30S ribosomal protein S3 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of asparagine, which comprises
(a) increasing or generating the activity of a 50S ribosomal protein L14 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of tyrosine, which comprises
(a) increasing or generating the activity of a ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating the activity of a acyl-CoA synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of tyrosine, which comprises
(a) increasing or generating the activity of a acyl-CoA synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a acyl-CoA synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of aspartate, which comprises
(a) increasing or generating the activity of a 2-oxoglutarate dehydrogenase E1 subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of 5-oxoproline, which comprises
(a) increasing or generating the activity of a 2-oxoglutarate dehydrogenase E1 subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of serine, which comprises
(a) increasing or generating the activity of a GDP-mannose dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of serine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of phenylalanine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of aspartate, which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of tyrosine, which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of citrulline, which comprises
(a) increasing or generating the activity of a lysyl-tRNA synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of asparagine, which comprises
(a) increasing or generating the activity of a ABC transporter component non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of aspartate, which comprises
(a) increasing or generating the activity of a glucose-1-phosphate cytidylyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of glycine, which comprises
(a) increasing or generating the activity of a CDP-glucose-4,6-dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating the activity of a enoyl-CoA hydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of alanine, which comprises
(a) increasing or generating the activity of a glutathione peroxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of asparagine, which comprises
(a) increasing or generating the activity of a aminotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of phenylalanine, which comprises
(a) increasing or generating the activity of a aminotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a NADH-quinone oxidoreductase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating the activity of a glutamate-ammonia-ligase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a glutamate-ammonia-ligase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a RNA polymerase sigma factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of phenylalanine, which comprises
(a) increasing or generating the activity of a hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of aspartate, which comprises
(a) increasing or generating the activity of a glutamate-ammonia-ligase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of serine, which comprises
(a) increasing or generating the activity of a glutamate-ammonia-ligase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a glutamate-ammonia-ligase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating the activity of a fumarylacetoacetate hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a fumarylacetoacetate hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of serine, which comprises
(a) increasing or generating the activity of a HesB/YadR/YfhF family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating the activity of a peptidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of aspartate, which comprises
(a) increasing or generating the activity of a L-aspartate oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a Chaperone protein CIpB non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of phenylalanine, which comprises
(a) increasing or generating the activity of a electron transfer flavoprotein subunit beta non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a electron transfer flavoprotein subunit beta non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of citrulline, which comprises
(a) increasing or generating the activity of a AX653549-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of 5-oxoproline, which comprises
(a) increasing or generating the activity of a AX653549-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of aspartate, which comprises
(a) increasing or generating the activity of a AY087308-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of 5-oxoproline, which comprises
(a) increasing or generating the activity of a AY087308-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of alanine, which comprises
(a) increasing or generating the activity of a threonine synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating the activity of a threonine synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a threonine synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a threonine synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a threonine synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of phenylalanine, which comprises
(a) increasing or generating the activity of a acetolactate synthase small subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of glycine, which comprises
(a) increasing or generating the activity of a glucose dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating the activity of a glucose dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a glucose dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a glucose dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of asparagine, which comprises
(a) increasing or generating the activity of a aspartate 1-decarboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of serine, which comprises
(a) increasing or generating the activity of a aspartate 1-decarboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of alanine, which comprises
(a) increasing or generating the activity of a serine protease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of serine, which comprises
(a) increasing or generating the activity of a serine protease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a serine protease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of serine, which comprises
(a) increasing or generating the activity of a acyl-CoA dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a acyl-CoA dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of tyrosine, which comprises
(a) increasing or generating the activity of a beta-galactosidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a beta-galactosidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of glycine, which comprises
(a) increasing or generating the activity of a 2,3-dihydroxyphenylpropionate 1,2-dioxygenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating the activity of a 2,3-dihydroxyphenylpropionate 1,2-dioxygenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a 2,3-dihydroxyphenylpropionate 1,2-dioxygenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a 2,3-dihydroxyphenylpropionate 1,2-dioxygenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating the activity of a b0391-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of asparagine, which comprises
(a) increasing or generating the activity of a ATP-binding component of a transport system non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of aspartate, which comprises
(a) increasing or generating the activity of a ATP-binding component of a transport system non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a ATP-binding component of a transport system non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a ATP-binding component of a transport system non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a ATP-binding component of a transport system non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating the activity of a b0456-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of aspartate, which comprises
(a) increasing or generating the activity of a membrane transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a membrane transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a membrane transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of homoserine, which comprises
(a) increasing or generating the activity of a b0518-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

A further embodiment of the present invention relates to a process for the production of aspartate, which comprises
(a) increasing or generating the activity of a isochorismate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a isochorismate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a isochorismate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a isochorismate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 3,4-dihydroxyphenylalanine (dopa), which comprises
(a) increasing or generating the activity of a isochorismate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4-dihydroxyphenylalanine (dopa) or a composition comprising 3,4-dihydroxyphenylalanine (dopa) in said non-human organism or in the culture medium surrounding said non-human organism.

A further embodiment of the present invention relates to a process for the production of 5-oxoproline, which comprises
(a) increasing or generating the activity of a isochorismate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of tyrosine, which comprises
(a) increasing or generating the activity of a zinc transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of tyrosine, which comprises
(a) increasing or generating the activity of a 3-deoxy-D-arabinoheptulosonate-7-phosphatesynthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of tyrosine, which comprises
(a) increasing or generating the activity of a b0801-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating the activity of a asparaginase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of glycine, which comprises
(a) increasing or generating the activity of a b0841-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of homoserine, which comprises
(a) increasing or generating the activity of a b0841-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

A further embodiment of the present invention relates to a process for the production of phenylalanine, which comprises
(a) increasing or generating the activity of a major facilitator superfamily transporter protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a major facilitator superfamily transporter protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a major facilitator superfamily transporter protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a major facilitator superfamily transporter protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of citrulline, which comprises
(a) increasing or generating the activity of a b0917-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of glycine, which comprises
(a) increasing or generating the activity of a b0917-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating the activity of a b0917-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of 5-oxoproline, which comprises
(a) increasing or generating the activity of a b0917-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of alanine, which comprises
(a) increasing or generating the activity of a methylglyoxal synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating the activity of a methylglyoxal synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a methylglyoxal synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating the activity of a phosphoanhydride phosphorylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of alanine, which comprises
(a) increasing or generating the activity of a b1003-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of aspartate, which comprises
(a) increasing or generating the activity of a b1003-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a b1003-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a b1003-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a b1003-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 3,4-dihydroxyphenylalanine (dopa), which comprises
(a) increasing or generating the activity of a b1003-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4-dihydroxyphenylalanine (dopa) or a composition comprising 3,4-dihydroxyphenylalanine (dopa) in said non-human organism or in the culture medium surrounding said non-human organism.

A further embodiment of the present invention relates to a process for the production of 5-oxoproline, which comprises
(a) increasing or generating the activity of a b1003-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of citrulline, which comprises
(a) increasing or generating the activity of a lipoprotein precursor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of 5-oxoproline, which comprises
(a) increasing or generating the activity of a lipoprotein precursor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of aspartate, which comprises
(a) increasing or generating the activity of a b1024-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of aspartate, which comprises
(a) increasing or generating the activity of a multidrug resistance protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of tyrosine, which comprises
(a) increasing or generating the activity of a multidrug resistance protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of serine, which comprises
(a) increasing or generating the activity of a b1137-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of phenylalanine, which comprises
(a) increasing or generating the activity of a b1155-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of phenylalanine, which comprises
(a) increasing or generating the activity of a b1163-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a b1163-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of citrulline, which comprises
(a) increasing or generating the activity of a b1179-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of alanine, which comprises
(a) increasing or generating the activity of a sodium/proton antiporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of serine, which comprises
(a) increasing or generating the activity of a sodium/proton antiporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of citrulline, which comprises
(a) increasing or generating the activity of a b1205-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of phenylalanine, which comprises
(a) increasing or generating the activity of a b1229-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a membrane protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of citrulline, which comprises
(a) increasing or generating the activity of a b1259-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of 5-oxoproline, which comprises
(a) increasing or generating the activity of a b1259-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of tyrosine, which comprises
(a) increasing or generating the activity of a tryptophan biosynthesis protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating the activity of a anthranilate synthase component I non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of glycine, which comprises
(a) increasing or generating the activity of a b1280-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of serine, which comprises
(a) increasing or generating the activity of a b1280-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of 5-oxoproline, which comprises
(a) increasing or generating the activity of a b1280-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of alanine, which comprises
(a) increasing or generating the activity of a glutamine synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of glycine, which comprises
(a) increasing or generating the activity of a glutamine synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating the activity of a aldehyde dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a aldehyde dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of citrulline, which comprises
(a) increasing or generating the activity of a ABC transporter substrate-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of citrulline, which comprises
(a) increasing or generating the activity of a b1330-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of phenylalanine, which comprises
(a) increasing or generating the activity of a ATP-dependent RNA helicase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of alanine, which comprises
(a) increasing or generating the activity of a restriction alleviation protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of glycine, which comprises
(a) increasing or generating the activity of a restriction alleviation protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of 5-oxoproline, which comprises
(a) increasing or generating the activity of a lipoprotein precursor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of alanine, which comprises
(a) increasing or generating the activity of a b1445-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of citrulline, which comprises
(a) increasing or generating the activity of a b1445-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of glycine, which comprises
(a) increasing or generating the activity of a b1445-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating the activity of a b1445-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a b1445-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of alanine, which comprises
(a) increasing or generating the activity of a NADP-dependent malic enzyme non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of glycine, which comprises
(a) increasing or generating the activity of a NADP-dependent malic enzyme non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating the activity of a NADP-dependent malic enzyme non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a NADP-dependent malic enzyme non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a NADP-dependent malic enzyme non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of asparagine, which comprises
(a) increasing or generating the activity of a b1522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of aspartate, which comprises
(a) increasing or generating the activity of a b1522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a b1522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a b1522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a b1522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a b1522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of tyrosine, which comprises
(a) increasing or generating the activity of a b1583-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating the activity of a Fe—S subunit of oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a acid shock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a acid shock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating the activity of a methylglyoxalase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of aspartate, which comprises
(a) increasing or generating the activity of a b1670-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of aspartate, which comprises
(a) increasing or generating the activity of a b1672-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of alanine, which comprises
(a) increasing or generating the activity of a arginine N-succinyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of aspartate, which comprises
(a) increasing or generating the activity of a ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of tyrosine, which comprises
(a) increasing or generating the activity of a ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating the activity of a transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of aspartate, which comprises
(a) increasing or generating the activity of a b1837-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of aspartate, which comprises
(a) increasing or generating the activity of a serine/threonine-protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of aspartate, which comprises
(a) increasing or generating the activity of a protease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of serine, which comprises
(a) increasing or generating the activity of a protease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of asparagine, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of tyrosine, which comprises
(a) increasing or generating the activity of a cytochrome c-type protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating the activity of a trehalose-phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a trehalose-phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of serine, which comprises
(a) increasing or generating the activity of a b1898-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of serine, which comprises
(a) increasing or generating the activity of a b1930-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating the activity of a transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of tyrosine, which comprises
(a) increasing or generating the activity of a O-antigen chain length determinant non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating the activity of a b2032-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of aspartate, which comprises
(a) increasing or generating the activity of a transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a uridine/cytidine kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of aspartate, which comprises
(a) increasing or generating the activity of a b2107-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of aspartate, which comprises
(a) increasing or generating the activity of a ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of citrulline, which comprises
(a) increasing or generating the activity of a ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of phenylalanine, which comprises
(a) increasing or generating the activity of a ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of glycine, which comprises
(a) increasing or generating the activity of a sn-glycerol-3-phosphate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of asparagine, which comprises
(a) increasing or generating the activity of a NADH dehydrogenase I chain I non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of aspartate, which comprises
(a) increasing or generating the activity of a b2360-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of serine, which comprises
(a) increasing or generating the activity of a D-serine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a b2399-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 3,4-dihydroxyphenylalanine (dopa), which comprises
(a) increasing or generating the activity of a b2399-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4-dihydroxyphenylalanine (dopa) or a composition comprising 3,4-dihydroxyphenylalanine (dopa) in said non-human organism or in the culture medium surrounding said non-human organism.

A further embodiment of the present invention relates to a process for the production of 5-oxoproline, which comprises
(a) increasing or generating the activity of a b2399-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of alanine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of aspartate, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a PTS system N-acetylmuramic acid-specific EIIBC component non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of citrulline, which comprises
(a) increasing or generating the activity of a b2474-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of glycine, which comprises
(a) increasing or generating the activity of a b2474-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of serine, which comprises
(a) increasing or generating the activity of a NADH dehydrogenase subunit N non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of 5-oxoproline, which comprises
(a) increasing or generating the activity of a NADH dehydrogenase subunit N non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of phenylalanine, which comprises
(a) increasing or generating the activity of a b2513-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a b2513-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of serine, which comprises
(a) increasing or generating the activity of a serine hydroxymethyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of phenylalanine, which comprises
(a) increasing or generating the activity of a chorismate mutase-T and prephenate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of alanine, which comprises
(a) increasing or generating the activity of a b2613-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating the activity of a b2673-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a b2673-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating the activity of a transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of alanine, which comprises
(a) increasing or generating the activity of a murein transglycosylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating the activity of a murein transglycosylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a murein transglycosylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of alanine, which comprises
(a) increasing or generating the activity of a b2739-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating the activity of a b2739-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a b2739-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of tyrosine, which comprises
(a) increasing or generating the activity of a 2-C-methyl-D-erythritol 4-phosphate cytidylyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a 2-C-methyl-D-erythritol 4-phosphate cytidylyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of phenylalanine, which comprises
(a) increasing or generating the activity of a phosphoadenosine phosphosulfate reductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of phenylalanine, which comprises
(a) increasing or generating the activity of a b2812-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a b2812-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a b2812-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating the activity of a amino-acid acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a amino-acid acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a amino-acid acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of aspartate, which comprises
(a) increasing or generating the activity of a prolipoprotein diacylglyceryl transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of tyrosine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating the activity of a b2849-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of glycine, which comprises
(a) increasing or generating the activity of a D-3-phosphoglycerate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of serine, which comprises
(a) increasing or generating the activity of a D-3-phosphoglycerate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating the activity of a arginine exporter protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of asparagine, which comprises
(a) increasing or generating the activity of a glycoprotease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of phenylalanine, which comprises
(a) increasing or generating the activity of a b3083-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a b3083-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a b3083-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating the activity of a b3098-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of glycine, which comprises
(a) increasing or generating the activity of a b3098-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of tyrosine, which comprises
(a) increasing or generating the activity of a b3121-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of tyrosine, which comprises
(a) increasing or generating the activity of a b3221-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of serine, which comprises
(a) increasing or generating the activity of a b3246-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of 5-oxoproline, which comprises
(a) increasing or generating the activity of a b3246-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of asparagine, which comprises
(a) increasing or generating the activity of a acetyl CoA carboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of glycine, which comprises
(a) increasing or generating the activity of a acetyl CoA carboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of serine, which comprises
(a) increasing or generating the activity of a acetyl CoA carboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of 5-oxoproline, which comprises
(a) increasing or generating the activity of a acetyl CoA carboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of alanine, which comprises
(a) increasing or generating the activity of a methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating the activity of a methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of alanine, which comprises
(a) increasing or generating the activity of a b3346-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of alanine, which comprises
(a) increasing or generating the activity of a ABC transporter ATP-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of glycine, which comprises
(a) increasing or generating the activity of a ABC transporter ATP-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of aspartate, which comprises
(a) increasing or generating the activity of a b3392-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a phosphoenolpyruvate carboxykinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a phosphoenolpyruvate carboxykinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of alanine, which comprises
(a) increasing or generating the activity of a b3410-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of serine, which comprises
(a) increasing or generating the activity of a b3509-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating the activity of a cellulose synthase catalytic subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a serine acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a serine acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating the activity of a phosphopantetheine adenylyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of asparagine, which comprises
(a) increasing or generating the activity of a phosphopantetheine adenylyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of phenylalanine, which comprises
(a) increasing or generating the activity of a phosphopantetheine adenylyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of glycine, which comprises
(a) increasing or generating the activity of a b3646-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of tyrosine, which comprises
(a) increasing or generating the activity of a acetolactate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of asparagine, which comprises
(a) increasing or generating the activity of a aspartate-ammonia ligase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of glycine, which comprises
(a) increasing or generating the activity of a dihydroxyacid dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating the activity of a dihydroxyacid dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of aspartate, which comprises
(a) increasing or generating the activity of a b3791-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of 5-oxoproline, which comprises
(a) increasing or generating the activity of a b3791-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of phenylalanine, which comprises
(a) increasing or generating the activity of a enterobacterial common antigen polymerase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of phenylalanine, which comprises
(a) increasing or generating the activity of a DNA helicase II non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of citrulline, which comprises
(a) increasing or generating the activity of a b3814-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of alanine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of aspartate, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of serine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating the activity of a b3989-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a b3989-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of 5-oxoproline, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of glycine, which comprises
(a) increasing or generating the activity of a aspartate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of serine, which comprises
(a) increasing or generating the activity of a aspartate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of alanine, which comprises
(a) increasing or generating the activity of a b4029-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of aspartate, which comprises
(a) increasing or generating the activity of a b4029-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of glycine, which comprises
(a) increasing or generating the activity of a b4029-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating the activity of a b4029-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a b4029-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a b4029-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a b4029-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of serine, which comprises
(a) increasing or generating the activity of a b4121-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of serine, which comprises
(a) increasing or generating the activity of a thioredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of alanine, which comprises
(a) increasing or generating the activity of a lysine decarboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of asparagine, which comprises
(a) increasing or generating the activity of a GM02LC11114-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of phenylalanine, which comprises
(a) increasing or generating the activity of a GM02LC11114-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of alanine, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating the activity of a histone H2A non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a histone H2A non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating the activity of a GM02LC17485-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a RNA binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a RNA binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating the activity of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of serine, which comprises
(a) increasing or generating the activity of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of alanine, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of glycine, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of serine, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of asparagine, which comprises
(a) increasing or generating the activity of a s_pp015018333r-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of phenylalanine, which comprises
(a) increasing or generating the activity of a s_pp015018333r-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a s_pp015018333r-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a s_pp015018333r-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of citrulline, which comprises
(a) increasing or generating the activity of a N-acetyl-gamma-glutamyl-phosphate reductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of asparagine, which comprises
(a) increasing or generating the activity of a agmatinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of tyrosine, which comprises
(a) increasing or generating the activity of a agmatinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating the activity of a ABC transporter ATP binding component non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating the activity of a flavodoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a flavodoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating the activity of a sll0281-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of serine, which comprises
(a) increasing or generating the activity of a uroporphyrin-III C-methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of phenylalanine, which comprises
(a) increasing or generating the activity of a urease subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a urease subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of citrulline, which comprises
(a) increasing or generating the activity of a glucokinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of serine, which comprises
(a) increasing or generating the activity of a permease protein of phosphate ABC transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of 5-oxoproline, which comprises
(a) increasing or generating the activity of a permease protein of phosphate ABC transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of aspartate, which comprises
(a) increasing or generating the activity of a oxireductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of glycine, which comprises
(a) increasing or generating the activity of a oxireductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of serine, which comprises
(a) increasing or generating the activity of a oxireductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a oxireductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating the activity of a malate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a malate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a malate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating the activity of a 3-deoxy-7-phosphoheptulonate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of serine, which comprises
(a) increasing or generating the activity of a glycogen synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating the activity of a carbon dioxide concentrating mechanism protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a carbon dioxide concentrating mechanism protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a carbon dioxide concentrating mechanism protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating the activity of a sll1032-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of serine, which comprises
(a) increasing or generating the activity of a phosphoribosylformyl glycinamidine synthase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating the activity of a 5′-nucleotidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of 5-oxoproline, which comprises
(a) increasing or generating the activity of a 5′-nucleotidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of asparagine, which comprises
(a) increasing or generating the activity of a glycogen (starch) synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of tyrosine, which comprises
(a) increasing or generating the activity of a glycogen (starch) synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating the activity of a CTP synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of glycine, which comprises
(a) increasing or generating the activity of a nitrate/nitrite transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating the activity of a nitrate/nitrite transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a nitrate/nitrite transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a nitrate/nitrite transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating the activity of a CDP-diacylglycerol-glycerol-3-phosphate 3-phosphatidyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of 5-oxoproline, which comprises
(a) increasing or generating the activity of a CDP-diacylglycerol-glycerol-3-phosphate 3-phosphatidyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of serine, which comprises
(a) increasing or generating the activity of a glutathione S-transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating the activity of a exopolyphosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a exopolyphosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a exopolyphosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating the activity of a ABC metal ion transporter substrate-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a ABC metal ion transporter substrate-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a sll1761-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a sll1761-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating the activity of a adenylate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of citrulline, which comprises
(a) increasing or generating the activity of a adenylate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of glycine, which comprises
(a) increasing or generating the activity of a adenylate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of serine, which comprises
(a) increasing or generating the activity of a adenylate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a adenylate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of glycine, which comprises
(a) increasing or generating the activity of a acyl transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating the activity of a acyl transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a acyl transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating the activity of a coproporphyrinogen III oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of citrulline, which comprises
(a) increasing or generating the activity of a coproporphyrinogen III oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of phenylalanine, which comprises
(a) increasing or generating the activity of a coproporphyrinogen III oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a coproporphyrinogen III oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a coproporphyrinogen III oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating the activity of a cation-transporting ATPase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of aspartate, which comprises
(a) increasing or generating the activity of a cation-transporting ATPase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of glycine, which comprises
(a) increasing or generating the activity of a glycosyl transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating the activity of a precorrin methylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a precorrin methylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of citrulline, which comprises
(a) increasing or generating the activity of a aldehyde dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of alanine, which comprises
(a) increasing or generating the activity of a bifunctional purine biosynthesis protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of serine, which comprises
(a) increasing or generating the activity of a bifunctional purine biosynthesis protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of 5-oxoproline, which comprises
(a) increasing or generating the activity of a bifunctional purine biosynthesis protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of asparagine, which comprises
(a) increasing or generating the activity of a Glu/Leu/Phe/Val dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of phenylalanine, which comprises
(a) increasing or generating the activity of a Glu/Leu/Phe/Val dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a Glu/Leu/Phe/Val dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating the activity of a malate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of alanine, which comprises
(a) increasing or generating the activity of a geranylgeranyl pyrophosphate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of asparagine, which comprises
(a) increasing or generating the activity of a geranylgeranyl pyrophosphate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of phenylalanine, which comprises
(a) increasing or generating the activity of a geranylgeranyl pyrophosphate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a geranylgeranyl pyrophosphate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a geranylgeranyl pyrophosphate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a geranylgeranyl pyrophosphate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of phenylalanine, which comprises
(a) increasing or generating the activity of a amine oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a tryptophan synthase alpha chain non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of ornithine, which comprises
(a) increasing or generating the activity of a argininosuccinate lyase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of ornithine or a composition comprising ornithine in said non-human organism or in the culture medium surrounding said non-human organism.

A further embodiment of the present invention relates to a process for the production of serine, which comprises
(a) increasing or generating the activity of a argininosuccinate lyase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of asparagine, which comprises
(a) increasing or generating the activity of a gamma-glutamyltranspeptidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of aspartate, which comprises
(a) increasing or generating the activity of a iron(III) dicitrate-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a iron(III) dicitrate-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of phenylalanine, which comprises
(a) increasing or generating the activity of a Na+/K+ transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a Na+/K+ transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating the activity of a 3-oxoacyl-[acyl-carrier-protein] synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a 3-oxoacyl-[acyl-carrier-protein] synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of phenylalanine, which comprises
(a) increasing or generating the activity of a Photosystem I reaction center subunit XI non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a Photosystem I reaction center subunit XI non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a Photosystem I reaction center subunit XI non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating the activity of a photosystem II protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a photosystem II protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a photosystem II protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a photosystem II protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of asparagine, which comprises
(a) increasing or generating the activity of a glutamine amidotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of citrulline, which comprises
(a) increasing or generating the activity of a phosphoadenosine phosphosulfate reductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of asparagine, which comprises
(a) increasing or generating the activity of a riboflavin biosynthesis protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of homoserine, which comprises
(a) increasing or generating the activity of a threonine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

A further embodiment of the present invention relates to a process for the production of tyrosine, which comprises
(a) increasing or generating the activity of a threonine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating the activity of a short-chain alcohol dehydrogenase family non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating the activity of a short-chain alcohol dehydrogenase family non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a short-chain alcohol dehydrogenase family non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating the activity of a Sec-independent protein translocase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a Sec-independent protein translocase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a Sec-independent protein translocase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of tyrosine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of asparagine, which comprises
(a) increasing or generating the activity of a amino acid ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of tyrosine, which comprises
(a) increasing or generating the activity of a TTC0768-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating the activity of a TTC0881-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of serine, which comprises
(a) increasing or generating the activity of a TTC0881-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of alanine, which comprises
(a) increasing or generating the activity of a metal-dependent hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of serine, which comprises
(a) increasing or generating the activity of a metal-dependent hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of phenylalanine, which comprises
(a) increasing or generating the activity of a multiple antibiotic resistance protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a multiple antibiotic resistance protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating the activity of a TTC1386-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of glycine, which comprises
(a) increasing or generating the activity of a TTC1386-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating the activity of a TTC1386-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a TTC1386-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a TTC1386-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating the activity of a homocitrate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating the activity of a homocitrate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a homocitrate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a homocitrate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a homocitrate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of aspartate, which comprises
(a) increasing or generating the activity of a oxidoreductase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of serine, which comprises
(a) increasing or generating the activity of a oxidoreductase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating the activity of a XM_{—}473199-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a XM_{—}473199-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a XM_{—}473199-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a XM_{—}473199-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of glycine, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of aspartate, which comprises
(a) increasing or generating the activity of a cell division control protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of tyrosine, which comprises
(a) increasing or generating the activity of a cell division control protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating the activity of a 3-deoxy-7-phosphoheptulonate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a 3-deoxy-7-phosphoheptulonate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a phosphate permease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of alanine, which comprises
(a) increasing or generating the activity of a serine/threonine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of glycine, which comprises
(a) increasing or generating the activity of a serine/threonine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of aspartate, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of homoserine, which comprises
(a) increasing or generating the activity of a Serine/threonine-protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

A further embodiment of the present invention relates to a process for the production of phenylalanine, which comprises
(a) increasing or generating the activity of a Serine/threonine-protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of 5-oxoproline, which comprises
(a) increasing or generating the activity of a Pre-mRNA-splicing factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of phenylalanine, which comprises
(a) increasing or generating the activity of a N-(5′-phosphoribosyl)anthranilate isomerase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a branched-chain amino acid permease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of serine, which comprises
(a) increasing or generating the activity of a ydr183w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating the activity of a ydr273w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of citrulline, which comprises
(a) increasing or generating the activity of a ydr273w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of aspartate, which comprises
(a) increasing or generating the activity of a ydr507c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of asparagine, which comprises
(a) increasing or generating the activity of a yer014w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of aspartate, which comprises
(a) increasing or generating the activity of a yer014w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of serine, which comprises
(a) increasing or generating the activity of a yer014w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of citrulline, which comprises
(a) increasing or generating the activity of a yer106w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of serine, which comprises
(a) increasing or generating the activity of a molecular chaperone portein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a molecular chaperone portein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating the activity of a YFL019C-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a YFL019C-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of alanine, which comprises
(a) increasing or generating the activity of a aminotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of glycine, which comprises
(a) increasing or generating the activity of a aminotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating the activity of a yfl054c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a yfl054c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of 5-oxoproline, which comprises
(a) increasing or generating the activity of a ygl096w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of tyrosine, which comprises
(a) increasing or generating the activity of a ygl237c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating the activity of a ygr221 c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of 5-oxoproline, which comprises
(a) increasing or generating the activity of a ygr221 c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of phenylalanine, which comprises
(a) increasing or generating the activity of a yhl013c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of phenylalanine, which comprises
(a) increasing or generating the activity of a yhr006w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a yhr006w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating the activity of a yhr207c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of citrulline, which comprises
(a) increasing or generating the activity of a 3-phosphoglycerate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of glycine, which comprises
(a) increasing or generating the activity of a 3-phosphoglycerate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of aspartate, which comprises
(a) increasing or generating the activity of a glutathione S-transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of citrulline, which comprises
(a) increasing or generating the activity of a glutathione S-transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of aspartate, which comprises
(a) increasing or generating the activity of a DnaJ-like chaperone non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a cystathionine gamma-synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of citrulline, which comprises
(a) increasing or generating the activity of a branched chain aminotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of aspartate, which comprises
(a) increasing or generating the activity of a polygalacturonase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a polygalacturonase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of alanine, which comprises
(a) increasing or generating the activity of a YKL038W-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of aspartate, which comprises
(a) increasing or generating the activity of a YKL038W-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of serine, which comprises
(a) increasing or generating the activity of a YKL038W-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of glycine, which comprises
(a) increasing or generating the activity of a aspartate aminotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of serine, which comprises
(a) increasing or generating the activity of a thioredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a thioredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating the activity of a sterol 24-C-methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of glycine, which comprises
(a) increasing or generating the activity of a sterol 24-C-methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of homoserine, which comprises
(a) increasing or generating the activity of a sterol 24-C-methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

A further embodiment of the present invention relates to a process for the production of 5-oxoproline, which comprises
(a) increasing or generating the activity of a sterol 24-C-methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of alanine, which comprises
(a) increasing or generating the activity of a yml083c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating the activity of a phosphate transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a phosphate transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of tyrosine, which comprises
(a) increasing or generating the activity of a ymr013c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of glycine, which comprises
(a) increasing or generating the activity of a purine biosynthesis protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating the activity of a mitochondrial inner membrane protease subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of homoserine, which comprises
(a) increasing or generating the activity of a YNL086W-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

A further embodiment of the present invention relates to a process for the production of tyrosine, which comprises
(a) increasing or generating the activity of a amino acid transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of homoserine, which comprises
(a) increasing or generating the activity of a homoserine O-acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

A further embodiment of the present invention relates to a process for the production of alanine, which comprises
(a) increasing or generating the activity of a metal ion transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of aspartate, which comprises
(a) increasing or generating the activity of a yol160w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of citrulline, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of phenylalanine, which comprises
(a) increasing or generating the activity of a histone acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating the activity of a histone acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of homoserine, which comprises
(a) increasing or generating the activity of a diacylglycerol O-acyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

A further embodiment of the present invention relates to a process for the production of serine, which comprises
(a) increasing or generating the activity of a diacylglycerol O-acyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of 5-oxoproline, which comprises
(a) increasing or generating the activity of a diacylglycerol O-acyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of glycine, which comprises
(a) increasing or generating the activity of a acetyl-CoA acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of serine, which comprises
(a) increasing or generating the activity of a citrate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating the activity of a ammonium transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating the activity of a Zm_{—}4842_BE510522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating the activity of a Zm_{—}4842_BE510522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating the activity of a Zm_{—}4842_BE510522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating the activity of a Zm_{—}4842_BE510522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of 3,4-dihydroxyphenylalanine (dopa), which comprises
(a) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3, whereby the respective line discloses in column 7 3,4-dihydroxyphenylalanine (dopa); or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, whereby the respective line discloses in column 7 3,4-dihydroxyphenylalanine (dopa);
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of 3,4-dihydroxyphenylalanine (dopa), or a composition comprising 3,4-dihydroxyphenylalanine (dopa) in said non-human organism or in the culture medium surrounding said non-human organism.

A further embodiment of the present invention relates to a process for the production of 5-oxoproline, which comprises
(a) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3, whereby the respective line discloses in column 7 5-oxoproline; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, whereby the respective line discloses in column 7 5-oxoproline;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of 5-oxoproline, or a composition comprising 5-oxoproline in said non-human organism or in the culture medium surrounding said non-human organism.

A further embodiment of the present invention relates to a process for the production of alanine, which comprises
(a) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3, whereby the respective line discloses in column 7 alanine; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, whereby the respective line discloses in column 7 alanine;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of alanine, or a composition comprising alanine in said non-human organism or in the culture medium surrounding said non-human organism.

A further embodiment of the present invention relates to a process for the production of asparagine, which comprises
(a) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3, whereby the respective line discloses in column 7 asparagine; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, whereby the respective line discloses in column 7 asparagine;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of asparagine, or a composition comprising asparagine in said non-human organism or in the culture medium surrounding said non-human organism.

A further embodiment of the present invention relates to a process for the production of aspartate, which comprises
(a) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3, whereby the respective line discloses in column 7 aspartate; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, whereby the respective line discloses in column 7 aspartate;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of aspartate, or a composition comprising aspartate in said non-human organism or in the culture medium surrounding said non-human organism.

A further embodiment of the present invention relates to a process for the production of citrulline, which comprises
(a) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3, whereby the respective line discloses in column 7 citrulline; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, whereby the respective line discloses in column 7 citrulline;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of citrulline, or a composition comprising citrulline in said non-human organism or in the culture medium surrounding said non-human organism.

A further embodiment of the present invention relates to a process for the production of glycine, which comprises
(a) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3, whereby the respective line discloses in column 7 glycine; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, whereby the respective line discloses in column 7 glycine;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of glycine, or a composition comprising glycine in said non-human organism or in the culture medium surrounding said non-human organism.

A further embodiment of the present invention relates to a process for the production of homoserine, which comprises
(a) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3, whereby the respective line discloses in column 7 homoserine; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, whereby the respective line discloses in column 7 homoserine;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of homoserine, or a composition comprising homoserine in said non-human organism or in the culture medium surrounding said non-human organism.

A further embodiment of the present invention relates to a process for the production of ornithine, which comprises
(a) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3, whereby the respective line discloses in column 7 ornithine; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, whereby the respective line discloses in column 7 ornithine;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of ornithine, or a composition comprising ornithine in said non-human organism or in the culture medium surrounding said non-human organism.

A further embodiment of the present invention relates to a process for the production of phenylalanine, which comprises
(a) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3, whereby the respective line discloses in column 7 phenylalanine; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, whereby the respective line discloses in column 7 phenylalanine;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of phenylalanine, or a composition comprising phenylalanine in said non-human organism or in the culture medium surrounding said non-human organism.

A further embodiment of the present invention relates to a process for the production of serine, which comprises
(a) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3, whereby the respective line discloses in column 7 serine; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, whereby the respective line discloses in column 7 serine;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of serine, or a composition comprising serine in said non-human organism or in the culture medium surrounding said non-human organism.

A further embodiment of the present invention relates to a process for the production of tyrosine, which comprises
(a) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3 , whereby the respective line discloses in column 7 tyrosine; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, whereby the respective line discloses in column 7 tyrosine;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of tyrosine, or a composition comprising tyrosine in said non-human organism or in the culture medium surrounding said non-human organism.

A further embodiment of the present invention relates to a process for the production of aspartate, which comprises
(a) increasing or generating one or more activities selected from the group consisting of 47266012-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of tyrosine, which comprises
(a) increasing or generating one or more activities selected from the group consisting of 47266012-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of 47266012-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of aspartate, which comprises
(a) increasing or generating one or more activities selected from the group consisting of 49747384_SOYBEAN-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of tyrosine, which comprises
(a) increasing or generating one or more activities selected from the group consisting of 49747384_SOYBEAN-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of 49747384_SOYBEAN-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of alanine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of citrulline, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of asparagine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of serine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of alanine, which comprises
(a) increasing or generating one or more activities selected from the group consisting of glucose-6-phosphate 1-dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating one or more activities selected from the group consisting of glucose-6-phosphate 1-dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of glucose-6-phosphate 1-dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating one or more activities selected from the group consisting of amino acid acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of citrulline, which comprises
(a) increasing or generating one or more activities selected from the group consisting of amino acid acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of phenylalanine, which comprises
(a) increasing or generating one or more activities selected from the group consisting of amino acid acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of citrulline, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of glycine, which comprises
(a) increasing or generating one or more activities selected from the group consisting of DNA-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of serine, which comprises
(a) increasing or generating one or more activities selected from the group consisting of DNA-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of phenylalanine, which comprises
(a) increasing or generating one or more activities selected from the group consisting of At1g17440-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of At1g17440-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating one or more activities selected from the group consisting of At1g19800-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of citrulline, which comprises
(a) increasing or generating one or more activities selected from the group consisting of At1g19800-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of tyrosine, which comprises
(a) increasing or generating one or more activities selected from the group consisting of At1g19800-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating one or more activities selected from the group consisting of cullin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of cullin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of homoserine, which comprises
(a) increasing or generating one or more activities selected from the group consisting of bifunctional aspartokinase/homoserine dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

A further embodiment of the present invention relates to a process for the production of aspartate, which comprises
(a) increasing or generating one or more activities selected from the group consisting of eukaryotic translation initiation factor 5 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of eukaryotic translation initiation factor 5 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of eukaryotic translation initiation factor 5 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of eukaryotic translation initiation factor 5 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of eukaryotic translation initiation factor 5 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of phenylalanine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of glycine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of alanine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of glycine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of tyrosine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of alanine, which comprises
(a) increasing or generating one or more activities selected from the group consisting of aminotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of 5-oxoproline, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of asparagine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of phenylalanine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating one or more activities selected from the group consisting of CBL-interacting protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of CBL-interacting protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of CBL-interacting protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of serine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of glycine, which comprises
(a) increasing or generating one or more activities selected from the group consisting of CBL-interacting protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of alanine, which comprises
(a) increasing or generating one or more activities selected from the group consisting of delta-1-pyrroline 5-carboxylase synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of glycine, which comprises
(a) increasing or generating one or more activities selected from the group consisting of delta-1-pyrroline 5-carboxylase synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of tyrosine, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating one or more activities selected from the group consisting of ankyrin repeat family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating one or more activities selected from the group consisting of thioredoxin family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of thioredoxin family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of thioredoxin family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating one or more activities selected from the group consisting of harpin-induced family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of harpin-induced family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of harpin-induced family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating one or more activities selected from the group consisting of DNA mismatch repair protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of phenylalanine, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of serine, which comprises
(a) increasing or generating one or more activities selected from the group consisting of eukaryotic translation initiation factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of glucose-6-phosphate 1-dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating one or more activities selected from the group consisting of glycosyl transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of glycosyl transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating one or more activities selected from the group consisting of auxin response factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of auxin response factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating one or more activities selected from the group consisting of monthiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of monthiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of phenylalanine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of phenylalanine, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of tyrosine, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of tyrosine, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of serine, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of alanine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of aspartate, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of citrulline, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of alanine, which comprises
(a) increasing or generating one or more activities selected from the group consisting of At4g32480-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of asparagine, which comprises
(a) increasing or generating one or more activities selected from the group consisting of At4g32480-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of glycine, which comprises
(a) increasing or generating one or more activities selected from the group consisting of At4g32480-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating one or more activities selected from the group consisting of At4g32480-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of At4g32480-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of At4g32480-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of citrulline, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of phenylalanine, which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of asparagine, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of phenylalanine, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of tyrosine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of 3,4-dihydroxyphenylalanine (dopa), which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4-dihydroxyphenylalanine (dopa) or a composition comprising 3,4-dihydroxyphenylalanine (dopa) in said non-human organism or in the culture medium surrounding said non-human organism.

A further embodiment of the present invention relates to a process for the production of phenylalanine, which comprises
(a) increasing or generating one or more activities selected from the group consisting of gibberellin 20-oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of gibberellin 20-oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of serine, which comprises
(a) increasing or generating one or more activities selected from the group consisting of integral membrane transporter family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of phenylalanine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of asparagine, which comprises
(a) increasing or generating one or more activities selected from the group consisting of At5g16650-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of aspartate, which comprises
(a) increasing or generating one or more activities selected from the group consisting of At5g16650-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of serine, which comprises
(a) increasing or generating one or more activities selected from the group consisting of At5g16650-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of 5-oxoproline, which comprises
(a) increasing or generating one or more activities selected from the group consisting of At5g16650-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of phenylalanine, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating one or more activities selected from the group consisting of at5g21910-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of serine, which comprises
(a) increasing or generating one or more activities selected from the group consisting of thioredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of alanine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of citrulline, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of citrulline, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of phenylalanine, which comprises
(a) increasing or generating one or more activities selected from the group consisting of RNA-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of RNA-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of alanine, which comprises
(a) increasing or generating one or more activities selected from the group consisting of zinc finger protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating one or more activities selected from the group consisting of zinc finger protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of zinc finger protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of 5-oxoproline, which comprises
(a) increasing or generating one or more activities selected from the group consisting of zinc finger protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of aspartate, which comprises
(a) increasing or generating one or more activities selected from the group consisting of CDP-diacylglycerol-serine O-phosphatidyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of 5-oxoproline, which comprises
(a) increasing or generating one or more activities selected from the group consisting of CDP-diacylglycerol-serine O-phosphatidyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of serine, which comprises
(a) increasing or generating one or more activities selected from the group consisting of Glycine cleavage system T aminomethyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of serine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating one or more activities selected from the group consisting of adenylylsulfate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of alanine, which comprises
(a) increasing or generating one or more activities selected from the group consisting of malic enzyme in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating one or more activities selected from the group consisting of malic enzyme in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of malic enzyme in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of alanine, which comprises
(a) increasing or generating one or more activities selected from the group consisting of sec-independent protein translocase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of alanine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of alanine, which comprises
(a) increasing or generating one or more activities selected from the group consisting of beta-hydroxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of aspartate, which comprises
(a) increasing or generating one or more activities selected from the group consisting of beta-hydroxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of serine, which comprises
(a) increasing or generating one or more activities selected from the group consisting of beta-hydroxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating one or more activities selected from the group consisting of elongation factor Tu in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of elongation factor Tu in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of elongation factor Tu in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of elongation factor Tu in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of elongation factor Tu in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of tyrosine, which comprises
(a) increasing or generating one or more activities selected from the group consisting of 30S ribosomal protein S3 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of asparagine, which comprises
(a) increasing or generating one or more activities selected from the group consisting of 50S ribosomal protein L14 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of tyrosine, which comprises
(a) increasing or generating one or more activities selected from the group consisting of ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating one or more activities selected from the group consisting of acyl-CoA synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of tyrosine, which comprises
(a) increasing or generating one or more activities selected from the group consisting of acyl-CoA synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of acyl-CoA synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of aspartate, which comprises
(a) increasing or generating one or more activities selected from the group consisting of 2-oxoglutarate dehydrogenase E1 subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of 5-oxoproline, which comprises
(a) increasing or generating one or more activities selected from the group consisting of 2-oxoglutarate dehydrogenase E1 subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of serine, which comprises
(a) increasing or generating one or more activities selected from the group consisting of GDP-mannose dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of serine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of phenylalanine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of aspartate, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of tyrosine, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of citrulline, which comprises
(a) increasing or generating one or more activities selected from the group consisting of lysyl-tRNA synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of asparagine, which comprises
(a) increasing or generating one or more activities selected from the group consisting of ABC transporter component in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of aspartate, which comprises
(a) increasing or generating one or more activities selected from the group consisting of glucose-1-phosphate cytidylyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of glycine, which comprises
(a) increasing or generating one or more activities selected from the group consisting of CDP-glucose-4,6-dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating one or more activities selected from the group consisting of enoyl-CoA hydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of alanine, which comprises
(a) increasing or generating one or more activities selected from the group consisting of glutathione peroxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of asparagine, which comprises
(a) increasing or generating one or more activities selected from the group consisting of aminotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of phenylalanine, which comprises
(a) increasing or generating one or more activities selected from the group consisting of aminotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of NADH-quinone oxidoreductase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating one or more activities selected from the group consisting of glutamate-ammonia-ligase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of glutamate-ammonia-ligase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of RNA polymerase sigma factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of phenylalanine, which comprises
(a) increasing or generating one or more activities selected from the group consisting of hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of aspartate, which comprises
(a) increasing or generating one or more activities selected from the group consisting of glutamate-ammonia-ligase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of serine, which comprises
(a) increasing or generating one or more activities selected from the group consisting of glutamate-ammonia-ligase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of glutamate-ammonia-ligase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating one or more activities selected from the group consisting of fumarylacetoacetate hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of fumarylacetoacetate hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of serine, which comprises
(a) increasing or generating one or more activities selected from the group consisting of HesB/YadR/YfhF family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating one or more activities selected from the group consisting of peptidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of aspartate, which comprises
(a) increasing or generating one or more activities selected from the group consisting of L-aspartate oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of Chaperone protein CIpB in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of phenylalanine, which comprises
(a) increasing or generating one or more activities selected from the group consisting of electron transfer flavoprotein subunit beta in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of electron transfer flavoprotein subunit beta in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of citrulline, which comprises
(a) increasing or generating one or more activities selected from the group consisting of AX653549-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of 5-oxoproline, which comprises
(a) increasing or generating one or more activities selected from the group consisting of AX653549-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of aspartate, which comprises
(a) increasing or generating one or more activities selected from the group consisting of AY087308-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of 5-oxoproline, which comprises
(a) increasing or generating one or more activities selected from the group consisting of AY087308-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of alanine, which comprises
(a) increasing or generating one or more activities selected from the group consisting of threonine synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating one or more activities selected from the group consisting of threonine synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of threonine synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of threonine synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of threonine synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of phenylalanine, which comprises
(a) increasing or generating one or more activities selected from the group consisting of acetolactate synthase small subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of glycine, which comprises
(a) increasing or generating one or more activities selected from the group consisting of glucose dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating one or more activities selected from the group consisting of glucose dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of glucose dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of glucose dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of asparagine, which comprises
(a) increasing or generating one or more activities selected from the group consisting of aspartate 1-decarboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of serine, which comprises
(a) increasing or generating one or more activities selected from the group consisting of aspartate 1-decarboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of alanine, which comprises
(a) increasing or generating one or more activities selected from the group consisting of serine protease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of serine, which comprises
(a) increasing or generating one or more activities selected from the group consisting of serine protease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of serine protease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of serine, which comprises
(a) increasing or generating one or more activities selected from the group consisting of acyl-CoA dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of acyl-CoA dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of tyrosine, which comprises
(a) increasing or generating one or more activities selected from the group consisting of beta-galactosidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of beta-galactosidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of glycine, which comprises
(a) increasing or generating one or more activities selected from the group consisting of 2,3-dihydroxyphenylpropionate 1,2-dioxygenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating one or more activities selected from the group consisting of 2,3-dihydroxyphenylpropionate 1,2-dioxygenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of 2,3-dihydroxyphenylpropionate 1,2-dioxygenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of 2,3-dihydroxyphenylpropionate 1,2-dioxygenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating one or more activities selected from the group consisting of b0391-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of asparagine, which comprises
(a) increasing or generating one or more activities selected from the group consisting of ATP-binding component of a transport system in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of aspartate, which comprises
(a) increasing or generating one or more activities selected from the group consisting of ATP-binding component of a transport system in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of ATP-binding component of a transport system in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of ATP-binding component of a transport system in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of ATP-binding component of a transport system in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating one or more activities selected from the group consisting of b0456-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of aspartate, which comprises
(a) increasing or generating one or more activities selected from the group consisting of membrane transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of membrane transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of membrane transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of homoserine, which comprises
(a) increasing or generating one or more activities selected from the group consisting of b0518-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

A further embodiment of the present invention relates to a process for the production of aspartate, which comprises
(a) increasing or generating one or more activities selected from the group consisting of isochorismate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of isochorismate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of isochorismate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of isochorismate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 3,4-dihydroxyphenylalanine (dopa), which comprises
(a) increasing or generating one or more activities selected from the group consisting of isochorismate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4-dihydroxyphenylalanine (dopa) or a composition comprising 3,4-dihydroxyphenylalanine (dopa) in said non-human organism or in the culture medium surrounding said non-human organism.

A further embodiment of the present invention relates to a process for the production of 5-oxoproline, which comprises
(a) increasing or generating one or more activities selected from the group consisting of isochorismate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of tyrosine, which comprises
(a) increasing or generating one or more activities selected from the group consisting of zinc transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of tyrosine, which comprises
(a) increasing or generating one or more activities selected from the group consisting of 3-deoxy-D-arabinoheptulosonate-7-phosphatesynthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of tyrosine, which comprises
(a) increasing or generating one or more activities selected from the group consisting of b0801-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating one or more activities selected from the group consisting of asparaginase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of glycine, which comprises
(a) increasing or generating one or more activities selected from the group consisting of b0841-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of homoserine, which comprises
(a) increasing or generating one or more activities selected from the group consisting of b0841-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

A further embodiment of the present invention relates to a process for the production of phenylalanine, which comprises
(a) increasing or generating one or more activities selected from the group consisting of major facilitator superfamily transporter protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of major facilitator superfamily transporter protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of major facilitator superfamily transporter protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of major facilitator superfamily transporter protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of citrulline, which comprises
(a) increasing or generating one or more activities selected from the group consisting of b0917-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of glycine, which comprises
(a) increasing or generating one or more activities selected from the group consisting of b0917-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating one or more activities selected from the group consisting of b0917-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of 5-oxoproline, which comprises
(a) increasing or generating one or more activities selected from the group consisting of b0917-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of alanine, which comprises
(a) increasing or generating one or more activities selected from the group consisting of methylglyoxal synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating one or more activities selected from the group consisting of methylglyoxal synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of methylglyoxal synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating one or more activities selected from the group consisting of phosphoanhydride phosphorylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of alanine, which comprises
(a) increasing or generating one or more activities selected from the group consisting of b1003-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of aspartate, which comprises
(a) increasing or generating one or more activities selected from the group consisting of b1003-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of b1003-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of b1003-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of b1003-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 3,4-dihydroxyphenylalanine (dopa), which comprises
(a) increasing or generating one or more activities selected from the group consisting of b1003-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4-dihydroxyphenylalanine (dopa) or a composition comprising 3,4-dihydroxyphenylalanine (dopa) in said non-human organism or in the culture medium surrounding said non-human organism.

A further embodiment of the present invention relates to a process for the production of 5-oxoproline, which comprises
(a) increasing or generating one or more activities selected from the group consisting of b1003-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of citrulline, which comprises
(a) increasing or generating one or more activities selected from the group consisting of lipoprotein precursor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of 5-oxoproline, which comprises
(a) increasing or generating one or more activities selected from the group consisting of lipoprotein precursor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of aspartate, which comprises
(a) increasing or generating one or more activities selected from the group consisting of b1024-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of aspartate, which comprises
(a) increasing or generating one or more activities selected from the group consisting of multidrug resistance protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of tyrosine, which comprises
(a) increasing or generating one or more activities selected from the group consisting of multidrug resistance protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of serine, which comprises
(a) increasing or generating one or more activities selected from the group consisting of b1137-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of phenylalanine, which comprises
(a) increasing or generating one or more activities selected from the group consisting of b1155-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of phenylalanine, which comprises
(a) increasing or generating one or more activities selected from the group consisting of b1163-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of b1163-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of citrulline, which comprises
(a) increasing or generating one or more activities selected from the group consisting of b1179-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of alanine, which comprises
(a) increasing or generating one or more activities selected from the group consisting of sodium/proton antiporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of serine, which comprises
(a) increasing or generating one or more activities selected from the group consisting of sodium/proton antiporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of citrulline, which comprises
(a) increasing or generating one or more activities selected from the group consisting of b1205-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of phenylalanine, which comprises
(a) increasing or generating one or more activities selected from the group consisting of b1229-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of membrane protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of citrulline, which comprises
(a) increasing or generating one or more activities selected from the group consisting of b1259-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of 5-oxoproline, which comprises
(a) increasing or generating one or more activities selected from the group consisting of b1259-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of tyrosine, which comprises
(a) increasing or generating one or more activities selected from the group consisting of tryptophan biosynthesis protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating one or more activities selected from the group consisting of anthranilate synthase component I in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of glycine, which comprises
(a) increasing or generating one or more activities selected from the group consisting of b1280-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of serine, which comprises
(a) increasing or generating one or more activities selected from the group consisting of b1280-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of 5-oxoproline, which comprises
(a) increasing or generating one or more activities selected from the group consisting of b1280-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of alanine, which comprises
(a) increasing or generating one or more activities selected from the group consisting of glutamine synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of glycine, which comprises
(a) increasing or generating one or more activities selected from the group consisting of glutamine synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating one or more activities selected from the group consisting of aldehyde dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of aldehyde dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of citrulline, which comprises
(a) increasing or generating one or more activities selected from the group consisting of ABC transporter substrate-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of citrulline, which comprises
(a) increasing or generating one or more activities selected from the group consisting of b1330-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of phenylalanine, which comprises
(a) increasing or generating one or more activities selected from the group consisting of ATPdependent RNA helicase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of alanine, which comprises
(a) increasing or generating one or more activities selected from the group consisting of restriction alleviation protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of glycine, which comprises
(a) increasing or generating one or more activities selected from the group consisting of restriction alleviation protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of 5-oxoproline, which comprises
(a) increasing or generating one or more activities selected from the group consisting of lipoprotein precursor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of alanine, which comprises
(a) increasing or generating one or more activities selected from the group consisting of b1445-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of citrulline, which comprises
(a) increasing or generating one or more activities selected from the group consisting of b1445-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of glycine, which comprises
(a) increasing or generating one or more activities selected from the group consisting of b1445-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating one or more activities selected from the group consisting of b1445-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of b1445-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of alanine, which comprises
(a) increasing or generating one or more activities selected from the group consisting of NADP-dependent malic enzyme in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of glycine, which comprises
(a) increasing or generating one or more activities selected from the group consisting of NADP-dependent malic enzyme in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating one or more activities selected from the group consisting of NADP-dependent malic enzyme in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of NADP-dependent malic enzyme in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of NADP-dependent malic enzyme in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of asparagine, which comprises
(a) increasing or generating one or more activities selected from the group consisting of b1522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of aspartate, which comprises
(a) increasing or generating one or more activities selected from the group consisting of b1522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of b1522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of b1522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of b1522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of b1522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of tyrosine, which comprises
(a) increasing or generating one or more activities selected from the group consisting of b1583-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating one or more activities selected from the group consisting of Fe—S subunit of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of acid shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of acid shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating one or more activities selected from the group consisting of methylglyoxalase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of aspartate, which comprises
(a) increasing or generating one or more activities selected from the group consisting of b1670-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of aspartate, which comprises
(a) increasing or generating one or more activities selected from the group consisting of b1672-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of alanine, which comprises
(a) increasing or generating one or more activities selected from the group consisting of arginine N-succinyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of aspartate, which comprises
(a) increasing or generating one or more activities selected from the group consisting of ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of tyrosine, which comprises
(a) increasing or generating one or more activities selected from the group consisting of ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating one or more activities selected from the group consisting of transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of aspartate, which comprises
(a) increasing or generating one or more activities selected from the group consisting of b1837-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of aspartate, which comprises
(a) increasing or generating one or more activities selected from the group consisting of serine/threonine-protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of aspartate, which comprises
(a) increasing or generating one or more activities selected from the group consisting of protease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of serine, which comprises
(a) increasing or generating one or more activities selected from the group consisting of protease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of asparagine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of tyrosine, which comprises
(a) increasing or generating one or more activities selected from the group consisting of cytochrome c-type protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating one or more activities selected from the group consisting of trehalose-phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of trehalose-phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of serine, which comprises
(a) increasing or generating one or more activities selected from the group consisting of b1898-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of serine, which comprises
(a) increasing or generating one or more activities selected from the group consisting of b1930-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating one or more activities selected from the group consisting of transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of tyrosine, which comprises
(a) increasing or generating one or more activities selected from the group consisting of O-antigen chain length determinant in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating one or more activities selected from the group consisting of b2032-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of aspartate, which comprises
(a) increasing or generating one or more activities selected from the group consisting of transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of uridine/cytidine kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of aspartate, which comprises
(a) increasing or generating one or more activities selected from the group consisting of b2107-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of aspartate, which comprises
(a) increasing or generating one or more activities selected from the group consisting of ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of citrulline, which comprises
(a) increasing or generating one or more activities selected from the group consisting of ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of phenylalanine, which comprises
(a) increasing or generating one or more activities selected from the group consisting of ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of glycine, which comprises
(a) increasing or generating one or more activities selected from the group consisting of sn-glycerol-3-phosphate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of asparagine, which comprises
(a) increasing or generating one or more activities selected from the group consisting of NADH dehydrogenase I chain I in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of aspartate, which comprises
(a) increasing or generating one or more activities selected from the group consisting of b2360-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of serine, which comprises
(a) increasing or generating one or more activities selected from the group consisting of D-serine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of b2399-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 3,4-dihydroxyphenylalanine (dopa), which comprises
(a) increasing or generating one or more activities selected from the group consisting of b2399-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4-dihydroxyphenylalanine (dopa) or a composition comprising 3,4-dihydroxyphenylalanine (dopa) in said non-human organism or in the culture medium surrounding said non-human organism.

A further embodiment of the present invention relates to a process for the production of 5-oxoproline, which comprises
(a) increasing or generating one or more activities selected from the group consisting of b2399-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of alanine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of aspartate, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of PTS system N-acetylmuramic acid-specific EIIBC component in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of citrulline, which comprises
(a) increasing or generating one or more activities selected from the group consisting of b2474-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of glycine, which comprises
(a) increasing or generating one or more activities selected from the group consisting of b2474-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of serine, which comprises
(a) increasing or generating one or more activities selected from the group consisting of NADH dehydrogenase subunit N in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of 5-oxoproline, which comprises
(a) increasing or generating one or more activities selected from the group consisting of NADH dehydrogenase subunit N in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of phenylalanine, which comprises
(a) increasing or generating one or more activities selected from the group consisting of b2513-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of b2513-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of serine, which comprises
(a) increasing or generating one or more activities selected from the group consisting of serine hydroxymethyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of phenylalanine, which comprises
(a) increasing or generating one or more activities selected from the group consisting of chorismate mutase-T and prephenate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of alanine, which comprises
(a) increasing or generating one or more activities selected from the group consisting of b2613-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating one or more activities selected from the group consisting of b2673-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of b2673-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating one or more activities selected from the group consisting of transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of alanine, which comprises
(a) increasing or generating one or more activities selected from the group consisting of murein transglycosylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating one or more activities selected from the group consisting of murein transglycosylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of murein transglycosylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of alanine, which comprises
(a) increasing or generating one or more activities selected from the group consisting of b2739-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating one or more activities selected from the group consisting of b2739-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of b2739-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of tyrosine, which comprises
(a) increasing or generating one or more activities selected from the group consisting of 2-C-methyl-D-erythritol 4-phosphate cytidylyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of 2-C-methyl-D-erythritol 4-phosphate cytidylyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of phenylalanine, which comprises
(a) increasing or generating one or more activities selected from the group consisting of phosphoadenosine phosphosulfate reductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of phenylalanine, which comprises
(a) increasing or generating one or more activities selected from the group consisting of b2812-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of b2812-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of b2812-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating one or more activities selected from the group consisting of amino-acid acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of amino-acid acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of amino-acid acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of aspartate, which comprises
(a) increasing or generating one or more activities selected from the group consisting of prolipoprotein diacylglyceryl transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of tyrosine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating one or more activities selected from the group consisting of b2849-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of glycine, which comprises
(a) increasing or generating one or more activities selected from the group consisting of D-3-phosphoglycerate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of serine, which comprises
(a) increasing or generating one or more activities selected from the group consisting of D-3-phosphoglycerate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating one or more activities selected from the group consisting of arginine exporter protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of asparagine, which comprises
(a) increasing or generating one or more activities selected from the group consisting of glycoprotease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of phenylalanine, which comprises
(a) increasing or generating one or more activities selected from the group consisting of b3083-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of b3083-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of b3083-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating one or more activities selected from the group consisting of b3098-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of glycine, which comprises
(a) increasing or generating one or more activities selected from the group consisting of b3098-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of tyrosine, which comprises
(a) increasing or generating one or more activities selected from the group consisting of b3121-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of tyrosine, which comprises
(a) increasing or generating one or more activities selected from the group consisting of b3221-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of serine, which comprises
(a) increasing or generating one or more activities selected from the group consisting of b3246-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of 5-oxoproline, which comprises
(a) increasing or generating one or more activities selected from the group consisting of b3246-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of asparagine, which comprises
(a) increasing or generating one or more activities selected from the group consisting of acetyl CoA carboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of glycine, which comprises
(a) increasing or generating one or more activities selected from the group consisting of acetyl CoA carboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of serine, which comprises
(a) increasing or generating one or more activities selected from the group consisting of acetyl CoA carboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of 5-oxoproline, which comprises
(a) increasing or generating one or more activities selected from the group consisting of acetyl CoA carboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of alanine, which comprises
(a) increasing or generating one or more activities selected from the group consisting of methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating one or more activities selected from the group consisting of methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of alanine, which comprises
(a) increasing or generating one or more activities selected from the group consisting of b3346-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of alanine, which comprises
(a) increasing or generating one or more activities selected from the group consisting of ABC transporter ATP-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of glycine, which comprises
(a) increasing or generating one or more activities selected from the group consisting of ABC transporter ATP-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of aspartate, which comprises
(a) increasing or generating one or more activities selected from the group consisting of b3392-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of phosphoenolpyruvate carboxykinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of phosphoenolpyruvate carboxykinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of alanine, which comprises
(a) increasing or generating one or more activities selected from the group consisting of b3410-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of serine, which comprises
(a) increasing or generating one or more activities selected from the group consisting of b3509-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating one or more activities selected from the group consisting of cellulose synthase catalytic subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of serine acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of serine acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating one or more activities selected from the group consisting of phosphopantetheine adenylyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of asparagine, which comprises
(a) increasing or generating one or more activities selected from the group consisting of phosphopantetheine adenylyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of phenylalanine, which comprises
(a) increasing or generating one or more activities selected from the group consisting of phosphopantetheine adenylyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of glycine, which comprises
(a) increasing or generating one or more activities selected from the group consisting of b3646-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of tyrosine, which comprises
(a) increasing or generating one or more activities selected from the group consisting of acetolactate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of asparagine, which comprises
(a) increasing or generating one or more activities selected from the group consisting of aspartate-ammonia ligase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of glycine, which comprises
(a) increasing or generating one or more activities selected from the group consisting of dihydroxyacid dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating one or more activities selected from the group consisting of dihydroxyacid dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of aspartate, which comprises
(a) increasing or generating one or more activities selected from the group consisting of b3791-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of 5-oxoproline, which comprises
(a) increasing or generating one or more activities selected from the group consisting of b3791-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of phenylalanine, which comprises
(a) increasing or generating one or more activities selected from the group consisting of enterobacterial common antigen polymerase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of phenylalanine, which comprises
(a) increasing or generating one or more activities selected from the group consisting of DNA helicase II in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of citrulline, which comprises
(a) increasing or generating one or more activities selected from the group consisting of b3814-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of alanine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of aspartate, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of serine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating one or more activities selected from the group consisting of b3989-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of b3989-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of 5-oxoproline, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of glycine, which comprises
(a) increasing or generating one or more activities selected from the group consisting of aspartate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of serine, which comprises
(a) increasing or generating one or more activities selected from the group consisting of aspartate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of alanine, which comprises
(a) increasing or generating one or more activities selected from the group consisting of b4029-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of aspartate, which comprises
(a) increasing or generating one or more activities selected from the group consisting of b4029-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of glycine, which comprises
(a) increasing or generating one or more activities selected from the group consisting of b4029-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating one or more activities selected from the group consisting of b4029-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of b4029-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of b4029-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of b4029-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of serine, which comprises
(a) increasing or generating one or more activities selected from the group consisting of b4121-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of serine, which comprises
(a) increasing or generating one or more activities selected from the group consisting of thioredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of alanine, which comprises
(a) increasing or generating one or more activities selected from the group consisting of lysine decarboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of asparagine, which comprises
(a) increasing or generating one or more activities selected from the group consisting of GM02LC11114-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of phenylalanine, which comprises
(a) increasing or generating one or more activities selected from the group consisting of GM02LC11114-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of alanine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating one or more activities selected from the group consisting of histone H2A in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of histone H2A in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating one or more activities selected from the group consisting of GM02LC17485-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of RNA binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of RNA binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of serine, which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of alanine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of glycine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of serine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of asparagine, which comprises
(a) increasing or generating one or more activities selected from the group consisting of s_pp015018333r-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of phenylalanine, which comprises
(a) increasing or generating one or more activities selected from the group consisting of s_pp015018333r-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of s_pp015018333r-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of s_pp015018333r-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of citrulline, which comprises
(a) increasing or generating one or more activities selected from the group consisting of N-acetyl-gamma-glutamyl-phosphate reductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of asparagine, which comprises
(a) increasing or generating one or more activities selected from the group consisting of agmatinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of tyrosine, which comprises
(a) increasing or generating one or more activities selected from the group consisting of agmatinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating one or more activities selected from the group consisting of ABC transporter ATP binding component in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating one or more activities selected from the group consisting of flavodoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of flavodoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating one or more activities selected from the group consisting of sll0281-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of serine, which comprises
(a) increasing or generating one or more activities selected from the group consisting of uroporphyrin-III C-methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of phenylalanine, which comprises
(a) increasing or generating one or more activities selected from the group consisting of urease subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of urease subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of citrulline, which comprises
(a) increasing or generating one or more activities selected from the group consisting of glucokinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of serine, which comprises
(a) increasing or generating one or more activities selected from the group consisting of permease protein of phosphate ABC transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of 5-oxoproline, which comprises
(a) increasing or generating one or more activities selected from the group consisting of permease protein of phosphate ABC transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of aspartate, which comprises
(a) increasing or generating one or more activities selected from the group consisting of oxireductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of glycine, which comprises
(a) increasing or generating one or more activities selected from the group consisting of oxireductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of serine, which comprises
(a) increasing or generating one or more activities selected from the group consisting of oxireductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of oxireductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating one or more activities selected from the group consisting of malate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of malate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of malate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating one or more activities selected from the group consisting of 3-deoxy-7-phosphoheptulonate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of serine, which comprises
(a) increasing or generating one or more activities selected from the group consisting of glycogen synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating one or more activities selected from the group consisting of carbon dioxide concentrating mechanism protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of carbon dioxide concentrating mechanism protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of carbon dioxide concentrating mechanism protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating one or more activities selected from the group consisting of sll1032-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of serine, which comprises
(a) increasing or generating one or more activities selected from the group consisting of phosphoribosylformyl glycinamidine synthase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating one or more activities selected from the group consisting of 5′-nucleotidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of 5-oxoproline, which comprises
(a) increasing or generating one or more activities selected from the group consisting of 5′-nucleotidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of asparagine, which comprises
(a) increasing or generating one or more activities selected from the group consisting of glycogen (starch) synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of tyrosine, which comprises
(a) increasing or generating one or more activities selected from the group consisting of glycogen (starch) synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating one or more activities selected from the group consisting of CTP synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of glycine, which comprises
(a) increasing or generating one or more activities selected from the group consisting of nitrate /nitrite transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating one or more activities selected from the group consisting of nitrate /nitrite transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of nitrate /nitrite transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of nitrate/nitrite transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating one or more activities selected from the group consisting of CDP-diacylglycerol-glycerol-3-phosphate 3-phosphatidyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of 5-oxoproline, which comprises
(a) increasing or generating one or more activities selected from the group consisting of CDP-diacylglycerol-glycerol-3-phosphate 3-phosphatidyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of serine, which comprises
(a) increasing or generating one or more activities selected from the group consisting of glutathione S-transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating one or more activities selected from the group consisting of exopolyphosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of exopolyphosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of exopolyphosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating one or more activities selected from the group consisting of ABC metal ion transporter substrate-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of ABC metal ion transporter substrate-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of sll1761-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of sll1761-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating one or more activities selected from the group consisting of adenylate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of citrulline, which comprises
(a) increasing or generating one or more activities selected from the group consisting of adenylate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of glycine, which comprises
(a) increasing or generating one or more activities selected from the group consisting of adenylate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of serine, which comprises
(a) increasing or generating one or more activities selected from the group consisting of adenylate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of adenylate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of glycine, which comprises
(a) increasing or generating one or more activities selected from the group consisting of acyl transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating one or more activities selected from the group consisting of acyl transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of acyl transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating one or more activities selected from the group consisting of coproporphyrinogen Ill oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of citrulline, which comprises
(a) increasing or generating one or more activities selected from the group consisting of coproporphyrinogen Ill oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of phenylalanine, which comprises
(a) increasing or generating one or more activities selected from the group consisting of coproporphyrinogen III oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of coproporphyrinogen III oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of coproporphyrinogen III oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating one or more activities selected from the group consisting of cation-transporting ATPase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of aspartate, which comprises
(a) increasing or generating one or more activities selected from the group consisting of cation-transporting ATPase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of glycine, which comprises
(a) increasing or generating one or more activities selected from the group consisting of glycosyl transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating one or more activities selected from the group consisting of precorrin methylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of precorrin methylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of citrulline, which comprises
(a) increasing or generating one or more activities selected from the group consisting of aldehyde dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of alanine, which comprises
(a) increasing or generating one or more activities selected from the group consisting of bifunctional purine biosynthesis protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of serine, which comprises
(a) increasing or generating one or more activities selected from the group consisting of bifunctional purine biosynthesis protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of 5-oxoproline, which comprises
(a) increasing or generating one or more activities selected from the group consisting of bifunctional purine biosynthesis protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of asparagine, which comprises
(a) increasing or generating one or more activities selected from the group consisting of Glu/Leu/Phe/Val dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of phenylalanine, which comprises
(a) increasing or generating one or more activities selected from the group consisting of Glu/Leu/Phe/Val dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of Glu/Leu/Phe/Val dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating one or more activities selected from the group consisting of malate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of alanine, which comprises
(a) increasing or generating one or more activities selected from the group consisting of geranylgeranyl pyrophosphate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of asparagine, which comprises
(a) increasing or generating one or more activities selected from the group consisting of geranylgeranyl pyrophosphate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of phenylalanine, which comprises
(a) increasing or generating one or more activities selected from the group consisting of geranylgeranyl pyrophosphate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of geranylgeranyl pyrophosphate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of geranylgeranyl pyrophosphate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of geranylgeranyl pyrophosphate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of phenylalanine, which comprises
(a) increasing or generating one or more activities selected from the group consisting of amine oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of tryptophan synthase alpha chain in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of ornithine, which comprises
(a) increasing or generating one or more activities selected from the group consisting of argininosuccinate lyase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of ornithine or a composition comprising ornithine in said non-human organism or in the culture medium surrounding said non-human organism.

A further embodiment of the present invention relates to a process for the production of serine, which comprises
(a) increasing or generating one or more activities selected from the group consisting of argininosuccinate lyase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of asparagine, which comprises
(a) increasing or generating one or more activities selected from the group consisting of gamma-glutamyltranspeptidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of aspartate, which comprises
(a) increasing or generating one or more activities selected from the group consisting of iron(III) dicitrate-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of iron(III) dicitrate-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of phenylalanine, which comprises
(a) increasing or generating one or more activities selected from the group consisting of Na+/K+ transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of Na+/K+ transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating one or more activities selected from the group consisting of 3-oxoacyl-[acyl-carrier-protein] synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of 3-oxoacyl-[acyl-carrier-protein] synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of phenylalanine, which comprises
(a) increasing or generating one or more activities selected from the group consisting of Photosystem I reaction center subunit XI in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of Photosystem I reaction center subunit XI in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of Photosystem I reaction center subunit XI in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating one or more activities selected from the group consisting of photosystem II protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of photosystem II protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of photosystem II protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of photosystem II protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of asparagine, which comprises
(a) increasing or generating one or more activities selected from the group consisting of glutamine amidotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of citrulline, which comprises
(a) increasing or generating one or more activities selected from the group consisting of phosphoadenosine phosphosulfate reductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of asparagine, which comprises
(a) increasing or generating one or more activities selected from the group consisting of riboflavin biosynthesis protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of homoserine, which comprises
(a) increasing or generating one or more activities selected from the group consisting of threonine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

A further embodiment of the present invention relates to a process for the production of tyrosine, which comprises
(a) increasing or generating one or more activities selected from the group consisting of threonine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating one or more activities selected from the group consisting of short-chain alcohol dehydrogenase family in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating one or more activities selected from the group consisting of short-chain alcohol dehydrogenase family in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of short-chain alcohol dehydrogenase family in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating one or more activities selected from the group consisting of Sec-independent protein translocase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of Sec-independent protein translocase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of Sec-independent protein translocase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of tyrosine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of asparagine, which comprises
(a) increasing or generating one or more activities selected from the group consisting of amino acid ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of tyrosine, which comprises
(a) increasing or generating one or more activities selected from the group consisting of TCC0768-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating one or more activities selected from the group consisting of TCC0881-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of serine, which comprises
(a) increasing or generating one or more activities selected from the group consisting of TCC0881-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of alanine, which comprises
(a) increasing or generating one or more activities selected from the group consisting of metal-dependent hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of serine, which comprises
(a) increasing or generating one or more activities selected from the group consisting of metal-dependent hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of phenylalanine, which comprises
(a) increasing or generating one or more activities selected from the group consisting of multiple antibiotic resistance protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of multiple antibiotic resistance protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating one or more activities selected from the group consisting of TTC1386-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of glycine, which comprises
(a) increasing or generating one or more activities selected from the group consisting of TTC1386-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating one or more activities selected from the group consisting of TTC1386-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of TTC1386-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of TCC1386-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating one or more activities selected from the group consisting of homocitrate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating one or more activities selected from the group consisting of homocitrate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of homocitrate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of homocitrate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of homocitrate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of aspartate, which comprises
(a) increasing or generating one or more activities selected from the group consisting of oxidoreductase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of serine, which comprises
(a) increasing or generating one or more activities selected from the group consisting of oxidoreductase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating one or more activities selected from the group consisting of XM_{—}473199-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of XM_{—}473199-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of XM_{—}473199-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of XM_{—}473199-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of glycine, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of aspartate, which comprises
(a) increasing or generating one or more activities selected from the group consisting of cell division control protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of tyrosine, which comprises
(a) increasing or generating one or more activities selected from the group consisting of cell division control protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of phenylalanine, which comprises
(a) increasing or generating one or more activities selected from the group consisting of 3-deoxy-7-phosphoheptulonate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of 3-deoxy-7-phosphoheptulonate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of phosphate permease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of alanine, which comprises
(a) increasing or generating one or more activities selected from the group consisting of serine/threonine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of glycine, which comprises
(a) increasing or generating one or more activities selected from the group consisting of serine/threonine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of aspartate, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of homoserine, which comprises
(a) increasing or generating one or more activities selected from the group consisting of Serine/threonine-protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

A further embodiment of the present invention relates to a process for the production of phenylalanine, which comprises
(a) increasing or generating one or more activities selected from the group consisting of Serine/threonine-protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of 5-oxoproline, which comprises
(a) increasing or generating one or more activities selected from the group consisting of Pre-mRNA-splicing factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of phenylalanine, which comprises
(a) increasing or generating one or more activities selected from the group consisting of N-(5′-phosphoribosyl)anthranilate isomerase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of branched-chain amino acid permease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of serine, which comprises
(a) increasing or generating one or more activities selected from the group consisting of ydr183w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating one or more activities selected from the group consisting of ydr273w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of citrulline, which comprises
(a) increasing or generating one or more activities selected from the group consisting of ydr273w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of aspartate, which comprises
(a) increasing or generating one or more activities selected from the group consisting of ydr507c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of asparagine, which comprises
(a) increasing or generating one or more activities selected from the group consisting of yer014w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

A further embodiment of the present invention relates to a process for the production of aspartate, which comprises
(a) increasing or generating one or more activities selected from the group consisting of yer014w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of serine, which comprises
(a) increasing or generating one or more activities selected from the group consisting of yer014w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of citrulline, which comprises
(a) increasing or generating one or more activities selected from the group consisting of yer106w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of serine, which comprises
(a) increasing or generating one or more activities selected from the group consisting of molecular chaperone portein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of molecular chaperone portein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating one or more activities selected from the group consisting of YFL019C-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of YFL019C-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of alanine, which comprises
(a) increasing or generating one or more activities selected from the group consisting of aminotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of glycine, which comprises
(a) increasing or generating one or more activities selected from the group consisting of aminotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating one or more activities selected from the group consisting of yfl054c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of yfl054c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of 5-oxoproline, which comprises
(a) increasing or generating one or more activities selected from the group consisting of ygl096w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of tyrosine, which comprises
(a) increasing or generating one or more activities selected from the group consisting of ygl237c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating one or more activities selected from the group consisting of ygr221c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of 5-oxoproline, which comprises
(a) increasing or generating one or more activities selected from the group consisting of ygr221c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of phenylalanine, which comprises
(a) increasing or generating one or more activities selected from the group consisting of yhl013c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of phenylalanine, which comprises
(a) increasing or generating one or more activities selected from the group consisting of yhr006w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of yhr006w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of aspartate, which comprises
(a) increasing or generating one or more activities selected from the group consisting of yhr207c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of citrulline, which comprises
(a) increasing or generating one or more activities selected from the group consisting of 3-phosphoglycerate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of glycine, which comprises
(a) increasing or generating one or more activities selected from the group consisting of 3-phosphoglycerate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of aspartate, which comprises
(a) increasing or generating one or more activities selected from the group consisting of glutathione S-transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of citrulline, which comprises
(a) increasing or generating one or more activities selected from the group consisting of glutathione S-transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of aspartate, which comprises
(a) increasing or generating one or more activities selected from the group consisting of DnaJ-like chaperone in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of cystathionine gamma-synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of citrulline, which comprises
(a) increasing or generating one or more activities selected from the group consisting of branched chain aminotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of aspartate, which comprises
(a) increasing or generating one or more activities selected from the group consisting of polygalacturonase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of polygalacturonase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of alanine, which comprises
(a) increasing or generating one or more activities selected from the group consisting of YKL038W-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of aspartate, which comprises
(a) increasing or generating one or more activities selected from the group consisting of YKL038W-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of serine, which comprises
(a) increasing or generating one or more activities selected from the group consisting of YKL038W-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of glycine, which comprises
(a) increasing or generating one or more activities selected from the group consisting of aspartate aminotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of serine, which comprises
(a) increasing or generating one or more activities selected from the group consisting of thioredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of thioredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of alanine, which comprises
(a) increasing or generating one or more activities selected from the group consisting of sterol 24-C-methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of glycine, which comprises
(a) increasing or generating one or more activities selected from the group consisting of sterol 24-C-methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of homoserine, which comprises
(a) increasing or generating one or more activities selected from the group consisting of sterol 24-C-methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

A further embodiment of the present invention relates to a process for the production of 5-oxoproline, which comprises
(a) increasing or generating one or more activities selected from the group consisting of sterol 24-C-methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of alanine, which comprises
(a) increasing or generating one or more activities selected from the group consisting of yml083c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of phenylalanine, which comprises
(a) increasing or generating one or more activities selected from the group consisting of phosphate transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of phosphate transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of tyrosine, which comprises
(a) increasing or generating one or more activities selected from the group consisting of ymr013c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of glycine, which comprises
(a) increasing or generating one or more activities selected from the group consisting of purine biosynthesis protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of phenylalanine, which comprises
(a) increasing or generating one or more activities selected from the group consisting of mitochondrial inner membrane protease subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of homoserine, which comprises
(a) increasing or generating one or more activities selected from the group consisting of YNL086W-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

A further embodiment of the present invention relates to a process for the production of tyrosine, which comprises
(a) increasing or generating one or more activities selected from the group consisting of amino acid transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of homoserine, which comprises
(a) increasing or generating one or more activities selected from the group consisting of homoserine O-acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

A further embodiment of the present invention relates to a process for the production of alanine, which comprises
(a) increasing or generating one or more activities selected from the group consisting of metal ion transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

A further embodiment of the present invention relates to a process for the production of aspartate, which comprises
(a) increasing or generating one or more activities selected from the group consisting of yol160w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of citrulline, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

A further embodiment of the present invention relates to a process for the production of phenylalanine, which comprises
(a) increasing or generating one or more activities selected from the group consisting of histone acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of tyrosine, which comprises
(a) increasing or generating one or more activities selected from the group consisting of histone acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of homoserine, which comprises
(a) increasing or generating one or more activities selected from the group consisting of diacylglycerol O-acyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

A further embodiment of the present invention relates to a process for the production of serine, which comprises
(a) increasing or generating one or more activities selected from the group consisting of diacylglycerol O-acyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of 5-oxoproline, which comprises
(a) increasing or generating one or more activities selected from the group consisting of diacylglycerol O-acyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of glycine, which comprises
(a) increasing or generating one or more activities selected from the group consisting of acetyl-CoA acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

A further embodiment of the present invention relates to a process for the production of serine, which comprises
(a) increasing or generating one or more activities selected from the group consisting of citrate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of aspartate, which comprises
(a) increasing or generating one or more activities selected from the group consisting of ammonium transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

A further embodiment of the present invention relates to a process for the production of phenylalanine, which comprises
(a) increasing or generating one or more activities selected from the group consisting of Zm_{—}4842_BE510522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

A further embodiment of the present invention relates to a process for the production of serine, which comprises
(a) increasing or generating one or more activities selected from the group consisting of Zm_{—}4842_BE510522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

A further embodiment of the present invention relates to a process for the production of tyrosine, which comprises
(a) increasing or generating one or more activities selected from the group consisting of Zm_{—}4842_BE510522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

A further embodiment of the present invention relates to a process for the production of 5-oxoproline, which comprises
(a) increasing or generating one or more activities selected from the group consisting of Zm_{—}4842_BE510522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

A further embodiment of the present invention relates to a process for the production of 3,4-dihydroxyphenylalanine (dopa), which comprises
(a1) increasing or generating the activity of a polypeptide comprising a polypeptide as depicted in the respective line in column 5 or 8 of Table II, application no. 3, or a homolog or fragment thereof, a consensus sequence or at least one polypeptide motif as depicted in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3, which is joined to a transit peptide; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, which is joined to a nucleic acid sequence encoding an organelle localization sequence, preferably a plastid or a mitochondrion localization sequence, especially a plastid localization sequence;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
   (wherein in the respective line in the respective Table in column 7 3,4-dihydroxyphenylalanine (dopa) is depicted)
   and
(b) growing the non-human organism under conditions which permit the production of 3,4-dihydroxyphenylalanine (dopa), or a composition comprising 3,4-dihydroxyphenylalanine (dopa) in said non-human organism or in the culture medium surrounding said non-human organism.

A further embodiment of the present invention relates to a process for the production of 5-oxoproline, which comprises
(a1) increasing or generating the activity of a polypeptide comprising a polypeptide as depicted in the respective line in column 5 or 8 of Table II, application no. 3, or a homolog or fragment thereof, a consensus sequence or at least one polypeptide motif as depicted in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3, which is joined to a transit peptide; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, which is joined to a nucleic acid sequence encoding an organelle localization sequence, preferably a plastid or a mitochondrion localization sequence, especially a plastid localization sequence;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
   (wherein in the respective line in the respective Table in column 7 5-oxoproline is depicted) and
(b) growing the non-human organism under conditions which permit the production of 5-oxoproline, or a composition comprising 5-oxoproline in said non-human organism or in the culture medium surrounding said non-human organism.

A further embodiment of the present invention relates to a process for the production of alanine, which comprises
(a1) increasing or generating the activity of a polypeptide comprising a polypeptide as depicted in the respective line in column 5 or 8 of Table II, application no. 3, or a homolog or fragment thereof, a consensus sequence or at least one polypeptide motif as depicted in column 8 of Table IV, application no. 3; or increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3, which is joined to a transit peptide; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, which is joined to a nucleic acid sequence encoding an organelle localization sequence, preferably a plastid or a mitochondrion localization sequence, especially a plastid localization sequence;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
   (wherein in the respective line in the respective Table in column 7 alanine is depicted)
   and
(b) growing the non-human organism under conditions which permit the production of alanine, or a composition comprising alanine in said non-human organism or in the culture medium surrounding said non-human organism.

A further embodiment of the present invention relates to a process for the production of asparagine, which comprises
(a1) increasing or generating the activity of a polypeptide comprising a polypeptide as depicted in the respective line in column 5 or 8 of Table II, application no. 3, or a homolog or fragment thereof, a consensus sequence or at least one polypeptide motif as depicted in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3, which is joined to a transit peptide; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, which is joined to a nucleic acid sequence encoding an organelle localization sequence, preferably a plastid or a mitochondrion localization sequence, especially a plastid localization sequence;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3; or increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
   (wherein in the respective line in the respective Table in column 7 asparagine is depicted)
and
(b) growing the non-human organism under conditions which permit the production of asparagine, or a composition comprising asparagine in said non-human organism or in the culture medium surrounding said non-human organism.

A further embodiment of the present invention relates to a process for the production of aspartate, which comprises
(a1) increasing or generating the activity of a polypeptide comprising a polypeptide as depicted in the respective line in column 5 or 8 of Table II, application no. 3, or a homolog or fragment thereof, a consensus sequence or at least one polypeptide motif as depicted in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3, which is joined to a transit peptide; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, which is joined to a nucleic acid sequence encoding an organelle localization sequence, preferably a plastid or a mitochondrion localization sequence, especially a plastid localization sequence;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
   (wherein in the respective line in the respective Table in column 7 aspartate is depicted)
   and
(b) growing the non-human organism under conditions which permit the production of aspartate, or a composition comprising aspartate in said non-human organism or in the culture medium surrounding said non-human organism.

A further embodiment of the present invention relates to a process for the production of citrulline, which comprises
(a1) increasing or generating the activity of a polypeptide comprising a polypeptide as depicted in the respective line in column 5 or 8 of Table II, application no. 3, or a homolog or fragment thereof, a consensus sequence or at least one polypeptide motif as depicted in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3, which is joined to a transit peptide; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, which is joined to a nucleic acid sequence encoding an organelle localization sequence, preferably a plastid or a mitochondrion localization sequence, especially a plastid localization sequence;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
   (wherein in the respective line in the respective Table in column 7 citrulline is depicted)
   and
(b) growing the non-human organism under conditions which permit the production of citrulline, or a composition comprising citrulline in said non-human organism or in the culture medium surrounding said non-human organism.

A further embodiment of the present invention relates to a process for the production of glycine, which comprises
(a1) increasing or generating the activity of a polypeptide comprising a polypeptide as depicted in the respective line in column 5 or 8 of Table II, application no. 3, or a homolog or fragment thereof, a consensus sequence or at least one polypeptide motif as depicted in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3, which is joined to a transit peptide; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, which is joined to a nucleic acid sequence encoding an organelle localization sequence, preferably a plastid or a mitochondrion localization sequence, especially a plastid localization sequence;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
   (wherein in the respective line in the respective Table in column 7 glycine is depicted)
   and
(b) growing the non-human organism under conditions which permit the production of glycine, or a composition comprising glycine in said non-human organism or in the culture medium surrounding said non-human organism.

A further embodiment of the present invention relates to a process for the production of homoserine, which comprises
(a1) increasing or generating the activity of a polypeptide comprising a polypeptide as depicted in the respective line in column 5 or 8 of Table II, application no. 3, or a homolog or fragment thereof, a consensus sequence or at least one polypeptide motif as depicted in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3, which is joined to a transit peptide; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, which is joined to a nucleic acid sequence encoding an organelle localization sequence, preferably a plastid or a mitochondrion localization sequence, especially a plastid localization sequence;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
   (wherein in the respective line in the respective Table in column 7 homoserine is depicted)
   and
(b) growing the non-human organism under conditions which permit the production of homoserine, or a composition comprising homoserine in said non-human organism or in the culture medium surrounding said non-human organism.

A further embodiment of the present invention relates to a process for the production of ornithine, which comprises
(a1) increasing or generating the activity of a polypeptide comprising a polypeptide as depicted in the respective line in column 5 or 8 of Table II, application no. 3, or a homolog or fragment thereof, a consensus sequence or at least one polypeptide motif as depicted in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3, which is joined to a transit peptide; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, which is joined to a nucleic acid sequence encoding an organelle localization sequence, preferably a plastid or a mitochondrion localization sequence, especially a plastid localization sequence;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
   (wherein in the respective line in the respective Table in column 7 orthinine is depicted)
   and
(b) growing the non-human organism under conditions which permit the production of ornithine, or a composition comprising ornithine in said non-human organism or in the culture medium surrounding said non-human organism.

A further embodiment of the present invention relates to a process for the production of phenylalanine, which comprises
(a1) increasing or generating the activity of a polypeptide comprising a polypeptide as depicted in the respective line in column 5 or 8 of Table II, application no. 3, or a homolog or fragment thereof, a consensus sequence or at least one polypeptide motif as depicted in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3, which is joined to a transit peptide; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, which is joined to a nucleic acid sequence encoding an organelle localization sequence, preferably a plastid or a mitochondrion localization sequence, especially a plastid localization sequence;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
   (wherein in the respective line in the respective Table in column 7 phenylalanine is depicted)
   and
(b) growing the non-human organism under conditions which permit the production of phenylalanine, or a composition comprising phenylalanine in said non-human organism or in the culture medium surrounding said non-human organism.

A further embodiment of the present invention relates to a process for the production of serine, which comprises
(a1) increasing or generating the activity of a polypeptide comprising a polypeptide as depicted in the respective line in column 5 or 8 of Table II, application no. 3, or a homolog or fragment thereof, a consensus sequence or at least one polypeptide motif as depicted in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3, which is joined to a transit peptide; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, which is joined to a nucleic acid sequence encoding an organelle localization sequence, preferably a plastid or a mitochondrion localization sequence, especially a plastid localization sequence;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
   (wherein in the respective line in the respective Table in column 7 serine is depicted)
   and
(b) growing the non-human organism under conditions which permit the production of serine, or a composition comprising serine in said non-human organism or in the culture medium surrounding said non-human organism.

A further embodiment of the present invention relates to a process for the production of tyrosine, which comprises
(a1) increasing or generating the activity of a polypeptide comprising a polypeptide as depicted in the respective line in column 5 or 8 of Table II, application no. 3, or a homolog or fragment thereof, a consensus sequence or at least one polypeptide motif as depicted in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3, which is joined to a transit peptide; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof, which is joined to a nucleic acid sequence encoding an organelle localization sequence, preferably a plastid or a mitochondrion localization sequence, especially a plastid localization sequence;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
   (wherein in the respective line in the respective Table in column 7 tyrosine is depicted)
   and
(b) growing the non-human organism under conditions which permit the production of tyrosine, or a composition comprising tyrosine in said non-human organism or in the culture medium surrounding said non-human organism.

Accordingly, the present invention relates to a process for the production of aspartate, which comprises
(a) increasing or generating the activity of a 47266012-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of tyrosine, which comprises
(a) increasing or generating the activity of a 47266012-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a 47266012-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of aspartate, which comprises
(a) increasing or generating the activity of a 49747384_SOYBEAN-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of tyrosine, which comprises
(a) increasing or generating the activity of a 49747384_SOYBEAN-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a 49747384_SOYBEAN-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of alanine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of citrulline, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of asparagine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of serine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of alanine, which comprises
(a) increasing or generating the activity of a glucose-6-phosphate 1-dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating the activity of a glucose-6-phosphate 1-dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a glucose-6-phosphate 1-dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating the activity of a amino acid acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of citrulline, which comprises
(a) increasing or generating the activity of a amino acid acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of phenylalanine, which comprises
(a) increasing or generating the activity of a amino acid acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of citrulline, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of glycine, which comprises
(a) increasing or generating the activity of a DNA-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of serine, which comprises
(a) increasing or generating the activity of a DNA-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of phenylalanine, which comprises
(a) increasing or generating the activity of a At1 g17440-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a At1g17440-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating the activity of a At1g19800-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of citrulline, which comprises
(a) increasing or generating the activity of a At1g19800-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of tyrosine, which comprises
(a) increasing or generating the activity of a At1g19800-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating the activity of a cullin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a cullin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of homoserine, which comprises
(a) increasing or generating the activity of a bifunctional aspartokinase/homoserine dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

Accordingly, the present invention relates to a process for the production of aspartate, which comprises
(a) increasing or generating the activity of a eukaryotic translation initiation factor 5 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a eukaryotic translation initiation factor 5 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a eukaryotic translation initiation factor 5 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a eukaryotic translation initiation factor 5 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a eukaryotic translation initiation factor 5 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of phenylalanine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of glycine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of alanine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of glycine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of tyrosine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of alanine, which comprises
(a) increasing or generating the activity of a aminotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of 5-oxoproline, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of asparagine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of phenylalanine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating the activity of a CBL-interacting protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a CBL-interacting protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a CBL-interacting protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of serine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of glycine, which comprises
(a) increasing or generating the activity of a CBL-interacting protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of alanine, which comprises
(a) increasing or generating the activity of a delta-1-pyrroline 5-carboxylase synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of glycine, which comprises
(a) increasing or generating the activity of a delta-1-pyrroline 5-carboxylase synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of tyrosine, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating the activity of a ankyrin repeat family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating the activity of a thioredoxin family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a thioredoxin family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a thioredoxin family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating the activity of a harpin-induced family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a harpin-induced family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a harpin-induced family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating the activity of a DNA mismatch repair protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of phenylalanine, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of serine, which comprises
(a) increasing or generating the activity of a eukaryotic translation initiation factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a glucose-6-phosphate 1-dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating the activity of a glycosyl transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a glycosyl transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating the activity of a auxin response factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a auxin response factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating the activity of a monthiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a monthiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of phenylalanine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of phenylalanine, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of tyrosine, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of tyrosine, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of serine, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of alanine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of aspartate, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of citrulline, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of alanine, which comprises
(a) increasing or generating the activity of a At4g32480-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of asparagine, which comprises
(a) increasing or generating the activity of a At4g32480-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of glycine, which comprises
(a) increasing or generating the activity of a At4g32480-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating the activity of a At4g32480-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a At4g32480-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a At4g32480-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of citrulline, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of phenylalanine, which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of asparagine, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of phenylalanine, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of tyrosine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of 3,4-dihydroxyphenylalanine (dopa), which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4-dihydroxyphenylalanine (dopa) or a composition comprising 3,4-dihydroxyphenylalanine (dopa) in said non-human organism or in the culture medium surrounding said non-human organism.

Accordingly, the present invention relates to a process for the production of phenylalanine, which comprises
(a) increasing or generating the activity of a gibberellin 20-oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a gibberellin 20-oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of serine, which comprises
(a) increasing or generating the activity of a integral membrane transporter family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of phenylalanine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of asparagine, which comprises
(a) increasing or generating the activity of a At5g16650-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of aspartate, which comprises
(a) increasing or generating the activity of a At5g16650-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of serine, which comprises
(a) increasing or generating the activity of a At5g16650-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of 5-oxoproline, which comprises
(a) increasing or generating the activity of a At5g16650-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of phenylalanine, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating the activity of a at5g21910-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of serine, which comprises
(a) increasing or generating the activity of a thioredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of alanine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of citrulline, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of citrulline, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of phenylalanine, which comprises
(a) increasing or generating the activity of a RNA-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a RNA-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of alanine, which comprises
(a) increasing or generating the activity of a zinc finger protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating the activity of a zinc finger protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a zinc finger protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of 5-oxoproline, which comprises
(a) increasing or generating the activity of a zinc finger protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of aspartate, which comprises
(a) increasing or generating the activity of a CDP-diacylglycerol-serine O-phosphatidyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of 5-oxoproline, which comprises
(a) increasing or generating the activity of a CDP-diacylglycerol-serine O-phosphatidyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of serine, which comprises
(a) increasing or generating the activity of a Glycine cleavage system T aminomethyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of serine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating the activity of a adenylylsulfate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of alanine, which comprises
(a) increasing or generating the activity of a malic enzyme in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating the activity of a malic enzyme in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a malic enzyme in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of alanine, which comprises
(a) increasing or generating the activity of a sec-independent protein translocase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of alanine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of alanine, which comprises
(a) increasing or generating the activity of a beta-hydroxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of aspartate, which comprises
(a) increasing or generating the activity of a beta-hydroxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of serine, which comprises
(a) increasing or generating the activity of a beta-hydroxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating the activity of a elongation factor Tu in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a elongation factor Tu in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a elongation factor Tu in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a elongation factor Tu in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a elongation factor Tu in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of tyrosine, which comprises
(a) increasing or generating the activity of a 30S ribosomal protein S3 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of asparagine, which comprises
(a) increasing or generating the activity of a 50S ribosomal protein L14 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of tyrosine, which comprises
(a) increasing or generating the activity of a ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating the activity of a acyl-CoA synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of tyrosine, which comprises
(a) increasing or generating the activity of a acyl-CoA synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a acyl-CoA synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of aspartate, which comprises
(a) increasing or generating the activity of a 2-oxoglutarate dehydrogenase E1 subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of 5-oxoproline, which comprises
(a) increasing or generating the activity of a 2-oxoglutarate dehydrogenase E1 subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of serine, which comprises
(a) increasing or generating the activity of a GDP-mannose dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of serine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of phenylalanine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of aspartate, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of tyrosine, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of citrulline, which comprises
(a) increasing or generating the activity of a lysyl-tRNA synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of asparagine, which comprises
(a) increasing or generating the activity of a ABC transporter component in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of aspartate, which comprises
(a) increasing or generating the activity of a glucose-1-phosphate cytidylyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of glycine, which comprises
(a) increasing or generating the activity of a CDP-glucose-4,6-dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating the activity of a enoyl-CoA hydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of alanine, which comprises
(a) increasing or generating the activity of a glutathione peroxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of asparagine, which comprises
(a) increasing or generating the activity of a aminotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of phenylalanine, which comprises
(a) increasing or generating the activity of a aminotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a NADH-quinone oxidoreductase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating the activity of a glutamate-ammonia-ligase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a glutamate-ammonia-ligase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a RNA polymerase sigma factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of phenylalanine, which comprises
(a) increasing or generating the activity of a hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of aspartate, which comprises
(a) increasing or generating the activity of a glutamate-ammonia-ligase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of serine, which comprises
(a) increasing or generating the activity of a glutamate-ammonia-ligase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a glutamate-ammonia-ligase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating the activity of a fumarylacetoacetate hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a fumarylacetoacetate hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of serine, which comprises
(a) increasing or generating the activity of a HesB/YadR/YfhF family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating the activity of a peptidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of aspartate, which comprises
(a) increasing or generating the activity of a L-aspartate oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a Chaperone protein CIpB in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of phenylalanine, which comprises
(a) increasing or generating the activity of a electron transfer flavoprotein subunit beta in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a electron transfer flavoprotein subunit beta in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of citrulline, which comprises
(a) increasing or generating the activity of a AX653549-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of 5-oxoproline, which comprises
(a) increasing or generating the activity of a AX653549-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of aspartate, which comprises
(a) increasing or generating the activity of a AY087308-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of 5-oxoproline, which comprises
(a) increasing or generating the activity of a AY087308-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of alanine, which comprises
(a) increasing or generating the activity of a threonine synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating the activity of a threonine synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a threonine synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a threonine synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a threonine synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of phenylalanine, which comprises
(a) increasing or generating the activity of a acetolactate synthase small subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of glycine, which comprises
(a) increasing or generating the activity of a glucose dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating the activity of a glucose dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a glucose dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a glucose dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of asparagine, which comprises
(a) increasing or generating the activity of a aspartate 1-decarboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of serine, which comprises
(a) increasing or generating the activity of a aspartate 1-decarboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of alanine, which comprises
(a) increasing or generating the activity of a serine protease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of serine, which comprises
(a) increasing or generating the activity of a serine protease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a serine protease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of serine, which comprises
(a) increasing or generating the activity of a acyl-CoA dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a acyl-CoA dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of tyrosine, which comprises
(a) increasing or generating the activity of a beta-galactosidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a beta-galactosidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of glycine, which comprises
(a) increasing or generating the activity of a 2,3-dihydroxyphenylpropionate 1,2-dioxygenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating the activity of a 2,3-dihydroxyphenylpropionate 1,2-dioxygenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a 2,3-dihydroxyphenylpropionate 1,2-dioxygenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a 2,3-dihydroxyphenylpropionate 1,2-dioxygenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating the activity of a b0391-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of asparagine, which comprises
(a) increasing or generating the activity of a ATP-binding component of a transport system in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of aspartate, which comprises
(a) increasing or generating the activity of a ATP-binding component of a transport system in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a ATP-binding component of a transport system in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a ATP-binding component of a transport system in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a ATP-binding component of a transport system in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating the activity of a b0456-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of aspartate, which comprises
(a) increasing or generating the activity of a membrane transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a membrane transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a membrane transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of homoserine, which comprises
(a) increasing or generating the activity of a b0518-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

Accordingly, the present invention relates to a process for the production of aspartate, which comprises
(a) increasing or generating the activity of a isochorismate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a isochorismate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a isochorismate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a isochorismate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 3,4-dihydroxyphenylalanine (dopa), which comprises
(a) increasing or generating the activity of a isochorismate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4-dihydroxyphenylalanine (dopa) or a composition comprising 3,4-dihydroxyphenylalanine (dopa) in said non-human organism or in the culture medium surrounding said non-human organism.

Accordingly, the present invention relates to a process for the production of 5-oxoproline, which comprises
(a) increasing or generating the activity of a isochorismate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of tyrosine, which comprises
(a) increasing or generating the activity of a zinc transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of tyrosine, which comprises
(a) increasing or generating the activity of a 3-deoxy-D-arabinoheptulosonate-7-phosphatesynthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of tyrosine, which comprises
(a) increasing or generating the activity of a b0801-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating the activity of a asparaginase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of glycine, which comprises
(a) increasing or generating the activity of a b0841-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of homoserine, which comprises
(a) increasing or generating the activity of a b0841-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

Accordingly, the present invention relates to a process for the production of phenylalanine, which comprises
(a) increasing or generating the activity of a major facilitator superfamily transporter protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a major facilitator superfamily transporter protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a major facilitator superfamily transporter protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a major facilitator superfamily transporter protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of citrulline, which comprises
(a) increasing or generating the activity of a b0917-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of glycine, which comprises
(a) increasing or generating the activity of a b0917-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating the activity of a b0917-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of 5-oxoproline, which comprises
(a) increasing or generating the activity of a b0917-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of alanine, which comprises
(a) increasing or generating the activity of a methylglyoxal synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating the activity of a methylglyoxal synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a methylglyoxal synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating the activity of a phosphoanhydride phosphorylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of alanine, which comprises
(a) increasing or generating the activity of a b1003-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of aspartate, which comprises
(a) increasing or generating the activity of a b1003-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a b1003-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a b1003-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a b1003-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 3,4-dihydroxyphenylalanine (dopa), which comprises
(a) increasing or generating the activity of a b1003-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4-dihydroxyphenylalanine (dopa) or a composition comprising 3,4-dihydroxyphenylalanine (dopa) in said non-human organism or in the culture medium surrounding said non-human organism.

Accordingly, the present invention relates to a process for the production of 5-oxoproline, which comprises
(a) increasing or generating the activity of a b1003-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of citrulline, which comprises
(a) increasing or generating the activity of a lipoprotein precursor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of 5-oxoproline, which comprises
(a) increasing or generating the activity of a lipoprotein precursor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of aspartate, which comprises
(a) increasing or generating the activity of a b1024-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of aspartate, which comprises
(a) increasing or generating the activity of a multidrug resistance protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of tyrosine, which comprises
(a) increasing or generating the activity of a multidrug resistance protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of serine, which comprises
(a) increasing or generating the activity of a b1137-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of phenylalanine, which comprises
(a) increasing or generating the activity of a b1155-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of phenylalanine, which comprises
(a) increasing or generating the activity of a b1163-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a b1163-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of citrulline, which comprises
(a) increasing or generating the activity of a b1179-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of alanine, which comprises
(a) increasing or generating the activity of a sodium/proton antiporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of serine, which comprises
(a) increasing or generating the activity of a sodium/proton antiporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of citrulline, which comprises
(a) increasing or generating the activity of a b1205-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of phenylalanine, which comprises
(a) increasing or generating the activity of a b1229-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a membrane protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of citrulline, which comprises
(a) increasing or generating the activity of a b1259-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of 5-oxoproline, which comprises
(a) increasing or generating the activity of a b1259-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of tyrosine, which comprises
(a) increasing or generating the activity of a tryptophan biosynthesis protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating the activity of a anthranilate synthase component I in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of glycine, which comprises
(a) increasing or generating the activity of a b1280-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of serine, which comprises
(a) increasing or generating the activity of a b1280-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of 5-oxoproline, which comprises
(a) increasing or generating the activity of a b1280-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of alanine, which comprises
(a) increasing or generating the activity of a glutamine synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of glycine, which comprises
(a) increasing or generating the activity of a glutamine synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating the activity of a aldehyde dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a aldehyde dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of citrulline, which comprises
(a) increasing or generating the activity of a ABC transporter substrate-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of citrulline, which comprises
(a) increasing or generating the activity of a b1330-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of phenylalanine, which comprises
(a) increasing or generating the activity of a ATP-dependent RNA helicase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of alanine, which comprises
(a) increasing or generating the activity of a restriction alleviation protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of glycine, which comprises
(a) increasing or generating the activity of a restriction alleviation protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of 5-oxoproline, which comprises
(a) increasing or generating the activity of a lipoprotein precursor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of alanine, which comprises
(a) increasing or generating the activity of a b1445-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of citrulline, which comprises
(a) increasing or generating the activity of a b1445-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of glycine, which comprises
(a) increasing or generating the activity of a b1445-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating the activity of a b1445-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a b1445-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of alanine, which comprises
(a) increasing or generating the activity of a NADP-dependent malic enzyme in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of glycine, which comprises
(a) increasing or generating the activity of a NADP-dependent malic enzyme in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating the activity of a NADP-dependent malic enzyme in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a NADP-dependent malic enzyme in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a NADP-dependent malic enzyme in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of asparagine, which comprises
(a) increasing or generating the activity of a b1522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of aspartate, which comprises
(a) increasing or generating the activity of a b1522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a b1522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a b1522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a b1522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a b1522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of tyrosine, which comprises
(a) increasing or generating the activity of a b1583-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating the activity of a Fe—S subunit of oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a acid shock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a acid shock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating the activity of a methylglyoxalase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of aspartate, which comprises
(a) increasing or generating the activity of a b1670-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of aspartate, which comprises
(a) increasing or generating the activity of a b1672-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of alanine, which comprises
(a) increasing or generating the activity of a arginine N-succinyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of aspartate, which comprises
(a) increasing or generating the activity of a ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of tyrosine, which comprises
(a) increasing or generating the activity of a ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating the activity of a transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of aspartate, which comprises
(a) increasing or generating the activity of a b1837-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of aspartate, which comprises
(a) increasing or generating the activity of a serine/threonine-protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of aspartate, which comprises
(a) increasing or generating the activity of a protease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of serine, which comprises
(a) increasing or generating the activity of a protease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of asparagine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of tyrosine, which comprises
(a) increasing or generating the activity of a cytochrome c-type protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating the activity of a trehalose-phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a trehalose-phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of serine, which comprises
(a) increasing or generating the activity of a b1898-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of serine, which comprises
(a) increasing or generating the activity of a b1930-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating the activity of a transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of tyrosine, which comprises
(a) increasing or generating the activity of a O-antigen chain length determinant in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating the activity of a b2032-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of aspartate, which comprises
(a) increasing or generating the activity of a transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a uridine/cytidine kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of aspartate, which comprises
(a) increasing or generating the activity of a b2107-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of aspartate, which comprises
(a) increasing or generating the activity of a ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of citrulline, which comprises
(a) increasing or generating the activity of a ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of phenylalanine, which comprises
(a) increasing or generating the activity of a ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of glycine, which comprises
(a) increasing or generating the activity of a sn-glycerol-3-phosphate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of asparagine, which comprises
(a) increasing or generating the activity of a NADH dehydrogenase I chain I in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of aspartate, which comprises
(a) increasing or generating the activity of a b2360-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of serine, which comprises
(a) increasing or generating the activity of a D-serine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a b2399-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 3,4-dihydroxyphenylalanine (dopa), which comprises
(a) increasing or generating the activity of a b2399-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4-dihydroxyphenylalanine (dopa) or a composition comprising 3,4-dihydroxyphenylalanine (dopa) in said non-human organism or in the culture medium surrounding said non-human organism.

Accordingly, the present invention relates to a process for the production of 5-oxoproline, which comprises
(a) increasing or generating the activity of a b2399-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of alanine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of aspartate, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a PTS system N-acetylmuramic acid-specific EIIBC component in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of citrulline, which comprises
(a) increasing or generating the activity of a b2474-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of glycine, which comprises
(a) increasing or generating the activity of a b2474-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of serine, which comprises
(a) increasing or generating the activity of a NADH dehydrogenase subunit N in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of 5-oxoproline, which comprises
(a) increasing or generating the activity of a NADH dehydrogenase subunit N in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of phenylalanine, which comprises
(a) increasing or generating the activity of a b2513-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a b2513-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of serine, which comprises
(a) increasing or generating the activity of a serine hydroxymethyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of phenylalanine, which comprises
(a) increasing or generating the activity of a chorismate mutase-T and prephenate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of alanine, which comprises
(a) increasing or generating the activity of a b2613-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating the activity of a b2673-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a b2673-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating the activity of a transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of alanine, which comprises
(a) increasing or generating the activity of a murein transglycosylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating the activity of a murein transglycosylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a murein transglycosylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of alanine, which comprises
(a) increasing or generating the activity of a b2739-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating the activity of a b2739-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a b2739-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of tyrosine, which comprises
(a) increasing or generating the activity of a 2-C-methyl-D-erythritol 4-phosphate cytidylyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a 2-C-methyl-D-erythritol 4-phosphate cytidylyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of phenylalanine, which comprises
(a) increasing or generating the activity of a phosphoadenosine phosphosulfate reductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of phenylalanine, which comprises
(a) increasing or generating the activity of a b2812-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a b2812-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a b2812-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating the activity of a amino-acid acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a amino-acid acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a amino-acid acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of aspartate, which comprises
(a) increasing or generating the activity of a prolipoprotein diacylglyceryl transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of tyrosine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating the activity of a b2849-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of glycine, which comprises
(a) increasing or generating the activity of a D-3-phosphoglycerate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of serine, which comprises
(a) increasing or generating the activity of a D-3-phosphoglycerate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating the activity of a arginine exporter protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of asparagine, which comprises
(a) increasing or generating the activity of a glycoprotease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of phenylalanine, which comprises
(a) increasing or generating the activity of a b3083-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a b3083-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a b3083-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating the activity of a b3098-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of glycine, which comprises
(a) increasing or generating the activity of a b3098-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of tyrosine, which comprises
(a) increasing or generating the activity of a b3121-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of tyrosine, which comprises
(a) increasing or generating the activity of a b3221-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of serine, which comprises
(a) increasing or generating the activity of a b3246-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of 5-oxoproline, which comprises
(a) increasing or generating the activity of a b3246-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of asparagine, which comprises
(a) increasing or generating the activity of a acetyl CoA carboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of glycine, which comprises
(a) increasing or generating the activity of a acetyl CoA carboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of serine, which comprises
(a) increasing or generating the activity of a acetyl CoA carboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of 5-oxoproline, which comprises
(a) increasing or generating the activity of a acetyl CoA carboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of alanine, which comprises
(a) increasing or generating the activity of a methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating the activity of a methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of alanine, which comprises
(a) increasing or generating the activity of a b3346-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of alanine, which comprises
(a) increasing or generating the activity of a ABC transporter ATP-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of glycine, which comprises
(a) increasing or generating the activity of a ABC transporter ATP-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of aspartate, which comprises
(a) increasing or generating the activity of a b3392-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a phosphoenolpyruvate carboxykinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a phosphoenolpyruvate carboxykinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of alanine, which comprises
(a) increasing or generating the activity of a b3410-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of serine, which comprises
(a) increasing or generating the activity of a b3509-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating the activity of a cellulose synthase catalytic subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a serine acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a serine acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating the activity of a phosphopantetheine adenylyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of asparagine, which comprises
(a) increasing or generating the activity of a phosphopantetheine adenylyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of phenylalanine, which comprises
(a) increasing or generating the activity of a phosphopantetheine adenylyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of glycine, which comprises
(a) increasing or generating the activity of a b3646-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of tyrosine, which comprises
(a) increasing or generating the activity of a acetolactate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of asparagine, which comprises
(a) increasing or generating the activity of a aspartate-ammonia ligase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of glycine, which comprises
(a) increasing or generating the activity of a dihydroxyacid dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating the activity of a dihydroxyacid dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of aspartate, which comprises
(a) increasing or generating the activity of a b3791-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of 5-oxoproline, which comprises
(a) increasing or generating the activity of a b3791-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of phenylalanine, which comprises
(a) increasing or generating the activity of a enterobacterial common antigen polymerase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of phenylalanine, which comprises
(a) increasing or generating the activity of a DNA helicase II in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of citrulline, which comprises
(a) increasing or generating the activity of a b3814-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of alanine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of aspartate, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of serine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating the activity of a b3989-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a b3989-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of 5-oxoproline, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of glycine, which comprises
(a) increasing or generating the activity of a aspartate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of serine, which comprises
(a) increasing or generating the activity of a aspartate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of alanine, which comprises
(a) increasing or generating the activity of a b4029-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of aspartate, which comprises
(a) increasing or generating the activity of a b4029-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of glycine, which comprises
(a) increasing or generating the activity of a b4029-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating the activity of a b4029-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a b4029-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a b4029-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a b4029-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of serine, which comprises
(a) increasing or generating the activity of a b4121-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of serine, which comprises
(a) increasing or generating the activity of a thioredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of alanine, which comprises
(a) increasing or generating the activity of a lysine decarboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of asparagine, which comprises
(a) increasing or generating the activity of a GM02LC11114-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of phenylalanine, which comprises
(a) increasing or generating the activity of a GM02LC11114-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of alanine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating the activity of a histone H2A in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a histone H2A in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating the activity of a GM02LC17485-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a RNA binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a RNA binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of serine, which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of alanine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of glycine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of serine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of asparagine, which comprises
(a) increasing or generating the activity of a s_pp015018333r-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of phenylalanine, which comprises
(a) increasing or generating the activity of a s_pp015018333r-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a s_pp015018333r-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a s_pp015018333r-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of citrulline, which comprises
(a) increasing or generating the activity of a N-acetyl-gamma-glutamyl-phosphate reductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of asparagine, which comprises
(a) increasing or generating the activity of a agmatinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of tyrosine, which comprises
(a) increasing or generating the activity of a agmatinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating the activity of a ABC transporter ATP binding component in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating the activity of a flavodoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a flavodoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating the activity of a sll0281-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of serine, which comprises
(a) increasing or generating the activity of a uroporphyrin-III C-methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of phenylalanine, which comprises
(a) increasing or generating the activity of a urease subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a urease subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of citrulline, which comprises
(a) increasing or generating the activity of a glucokinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of serine, which comprises
(a) increasing or generating the activity of a permease protein of phosphate ABC transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of 5-oxoproline, which comprises
(a) increasing or generating the activity of a permease protein of phosphate ABC transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of aspartate, which comprises
(a) increasing or generating the activity of a oxireductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of glycine, which comprises
(a) increasing or generating the activity of a oxireductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of serine, which comprises
(a) increasing or generating the activity of a oxireductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a oxireductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating the activity of a malate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a malate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a malate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating the activity of a 3-deoxy-7-phosphoheptulonate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of serine, which comprises
(a) increasing or generating the activity of a glycogen synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating the activity of a carbon dioxide concentrating mechanism protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a carbon dioxide concentrating mechanism protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a carbon dioxide concentrating mechanism protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating the activity of a sll1032-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of serine, which comprises
(a) increasing or generating the activity of a phosphoribosylformyl glycinamidine synthase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating the activity of a 5′-nucleotidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of 5-oxoproline, which comprises
(a) increasing or generating the activity of a 5′-nucleotidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of asparagine, which comprises
(a) increasing or generating the activity of a glycogen (starch) synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of tyrosine, which comprises
(a) increasing or generating the activity of a glycogen (starch) synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating the activity of a CTP synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of glycine, which comprises
(a) increasing or generating the activity of a nitrate/nitrite transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating the activity of a nitrate/nitrite transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a nitrate/nitrite transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a nitrate/nitrite transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating the activity of a CDP-diacylglycerol-glycerol-3-phosphate 3-phosphatidyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of 5-oxoproline, which comprises
(a) increasing or generating the activity of a CDP-diacylglycerol-glycerol-3-phosphate 3-phosphatidyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of serine, which comprises
(a) increasing or generating the activity of a glutathione S-transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating the activity of a exopolyphosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a exopolyphosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a exopolyphosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating the activity of a ABC metal ion transporter substrate-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a ABC metal ion transporter substrate-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a sll1761-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a sll1761-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating the activity of a adenylate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of citrulline, which comprises
(a) increasing or generating the activity of a adenylate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of glycine, which comprises
(a) increasing or generating the activity of a adenylate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of serine, which comprises
(a) increasing or generating the activity of a adenylate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a adenylate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of glycine, which comprises
(a) increasing or generating the activity of a acyl transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating the activity of a acyl transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a acyl transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating the activity of a coproporphyrinogen III oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of citrulline, which comprises
(a) increasing or generating the activity of a coproporphyrinogen III oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of phenylalanine, which comprises
(a) increasing or generating the activity of a coproporphyrinogen III oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a coproporphyrinogen III oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a coproporphyrinogen Ill oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating the activity of a cation-transporting ATPase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of aspartate, which comprises
(a) increasing or generating the activity of a cation-transporting ATPase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of glycine, which comprises
(a) increasing or generating the activity of a glycosyl transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating the activity of a precorrin methylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a precorrin methylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of citrulline, which comprises
(a) increasing or generating the activity of a aldehyde dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of alanine, which comprises
(a) increasing or generating the activity of a bifunctional purine biosynthesis protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of serine, which comprises
(a) increasing or generating the activity of a bifunctional purine biosynthesis protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of 5-oxoproline, which comprises
(a) increasing or generating the activity of a bifunctional purine biosynthesis protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of asparagine, which comprises
(a) increasing or generating the activity of a Glu/Leu/Phe/Val dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of phenylalanine, which comprises
(a) increasing or generating the activity of a Glu/Leu/Phe/Val dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a Glu/Leu/Phe/Val dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating the activity of a malate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of alanine, which comprises
(a) increasing or generating the activity of a geranylgeranyl pyrophosphate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of asparagine, which comprises
(a) increasing or generating the activity of a geranylgeranyl pyrophosphate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of phenylalanine, which comprises
(a) increasing or generating the activity of a geranylgeranyl pyrophosphate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a geranylgeranyl pyrophosphate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a geranylgeranyl pyrophosphate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a geranylgeranyl pyrophosphate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of phenylalanine, which comprises
(a) increasing or generating the activity of a amine oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a tryptophan synthase alpha chain in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of ornithine, which comprises
(a) increasing or generating the activity of a argininosuccinate lyase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of ornithine or a composition comprising ornithine in said non-human organism or in the culture medium surrounding said non-human organism.

Accordingly, the present invention relates to a process for the production of serine, which comprises
(a) increasing or generating the activity of a argininosuccinate lyase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of asparagine, which comprises
(a) increasing or generating the activity of a gamma-glutamyltranspeptidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of aspartate, which comprises
(a) increasing or generating the activity of a iron(III) dicitrate-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a iron(III) dicitrate-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of phenylalanine, which comprises
(a) increasing or generating the activity of a Na+/K+ transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a Na+/K+ transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating the activity of a 3-oxoacyl-[acyl-carrier-protein] synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a 3-oxoacyl-[acyl-carrier-protein] synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of phenylalanine, which comprises
(a) increasing or generating the activity of a Photosystem I reaction center subunit XI in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a Photosystem I reaction center subunit XI in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a Photosystem I reaction center subunit XI in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating the activity of a photosystem II protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a photosystem II protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a photosystem II protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a photosystem II protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of asparagine, which comprises
(a) increasing or generating the activity of a glutamine amidotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of citrulline, which comprises
(a) increasing or generating the activity of a phosphoadenosine phosphosulfate reductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of asparagine, which comprises
(a) increasing or generating the activity of a riboflavin biosynthesis protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of homoserine, which comprises
(a) increasing or generating the activity of a threonine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

Accordingly, the present invention relates to a process for the production of tyrosine, which comprises
(a) increasing or generating the activity of a threonine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating the activity of a short-chain alcohol dehydrogenase family in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating the activity of a short-chain alcohol dehydrogenase family in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a short-chain alcohol dehydrogenase family in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating the activity of a Sec-independent protein translocase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a Sec-independent protein translocase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a Sec-independent protein translocase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of tyrosine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of asparagine, which comprises
(a) increasing or generating the activity of a amino acid ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of tyrosine, which comprises
(a) increasing or generating the activity of a TTC0768-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating the activity of a TTC0881-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of serine, which comprises
(a) increasing or generating the activity of a TTC0881-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of alanine, which comprises
(a) increasing or generating the activity of a metal-dependent hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of serine, which comprises
(a) increasing or generating the activity of a metal-dependent hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of phenylalanine, which comprises
(a) increasing or generating the activity of a multiple antibiotic resistance protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a multiple antibiotic resistance protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating the activity of a TTC1386-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of glycine, which comprises
(a) increasing or generating the activity of a TTC1386-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating the activity of a TTC1386-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a TTC1386-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a TTC1386-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating the activity of a homocitrate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating the activity of a homocitrate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a homocitrate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a homocitrate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a homocitrate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of aspartate, which comprises
(a) increasing or generating the activity of a oxidoreductase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of serine, which comprises
(a) increasing or generating the activity of a oxidoreductase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating the activity of a XM_{—}473199-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a XM_{—}473199-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a XM_{—}473199-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a XM_{—}473199-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of glycine, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of aspartate, which comprises
(a) increasing or generating the activity of a cell division control protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of tyrosine, which comprises
(a) increasing or generating the activity of a cell division control protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of phenylalanine, which comprises
(a) increasing or generating the activity of a 3-deoxy-7-phosphoheptulonate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a 3-deoxy-7-phosphoheptulonate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a phosphate permease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of alanine, which comprises
(a) increasing or generating the activity of a serine/threonine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of glycine, which comprises
(a) increasing or generating the activity of a serine/threonine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of aspartate, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of homoserine, which comprises
(a) increasing or generating the activity of a Serine/threonine-protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

Accordingly, the present invention relates to a process for the production of phenylalanine, which comprises
(a) increasing or generating the activity of a Serine/threonine-protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of 5-oxoproline, which comprises
(a) increasing or generating the activity of a Pre-mRNA-splicing factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of phenylalanine, which comprises
(a) increasing or generating the activity of a N-(5′-phosphoribosyl)anthranilate isomerase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a branched-chain amino acid permease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of serine, which comprises
(a) increasing or generating the activity of a ydr183w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating the activity of a ydr273w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of citrulline, which comprises
(a) increasing or generating the activity of a ydr273w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of aspartate, which comprises
(a) increasing or generating the activity of a ydr507c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of asparagine, which comprises
(a) increasing or generating the activity of a yer014w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of asparagine or a composition comprising asparagine in said non-human organism or in the culture medium surrounding said non-human organism.

Accordingly, the present invention relates to a process for the production of aspartate, which comprises
(a) increasing or generating the activity of a yer014w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of serine, which comprises
(a) increasing or generating the activity of a yer014w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of citrulline, which comprises
(a) increasing or generating the activity of a yer106w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of serine, which comprises
(a) increasing or generating the activity of a molecular chaperone portein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a molecular chaperone portein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating the activity of a YFL019C-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a YFL019C-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of alanine, which comprises
(a) increasing or generating the activity of a aminotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of glycine, which comprises
(a) increasing or generating the activity of a aminotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating the activity of a yfl054c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a yfl054c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of 5-oxoproline, which comprises
(a) increasing or generating the activity of a ygl096w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of tyrosine, which comprises
(a) increasing or generating the activity of a ygl237c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating the activity of a ygr221c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of 5-oxoproline, which comprises
(a) increasing or generating the activity of a ygr221c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of phenylalanine, which comprises
(a) increasing or generating the activity of a yhl013c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of phenylalanine, which comprises
(a) increasing or generating the activity of a yhr006w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a yhr006w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of aspartate, which comprises
(a) increasing or generating the activity of a yhr207c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of citrulline, which comprises
(a) increasing or generating the activity of a 3-phosphoglycerate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of glycine, which comprises
(a) increasing or generating the activity of a 3-phosphoglycerate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of aspartate, which comprises
(a) increasing or generating the activity of a glutathione S-transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of citrulline, which comprises
(a) increasing or generating the activity of a glutathione S-transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of aspartate, which comprises
(a) increasing or generating the activity of a DnaJ-like chaperone in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a cystathionine gamma-synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of citrulline, which comprises
(a) increasing or generating the activity of a branched chain aminotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of aspartate, which comprises
(a) increasing or generating the activity of a polygalacturonase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a polygalacturonase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of alanine, which comprises
(a) increasing or generating the activity of a YKL038W-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of aspartate, which comprises
(a) increasing or generating the activity of a YKL038W-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of serine, which comprises
(a) increasing or generating the activity of a YKL038W-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of glycine, which comprises
(a) increasing or generating the activity of a aspartate aminotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of serine, which comprises
(a) increasing or generating the activity of a thioredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a thioredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of alanine, which comprises
(a) increasing or generating the activity of a sterol 24-C-methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of glycine, which comprises
(a) increasing or generating the activity of a sterol 24-C-methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of homoserine, which comprises
(a) increasing or generating the activity of a sterol 24-C-methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

Accordingly, the present invention relates to a process for the production of 5-oxoproline, which comprises
(a) increasing or generating the activity of a sterol 24-C-methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of alanine, which comprises
(a) increasing or generating the activity of a yml083c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of phenylalanine, which comprises
(a) increasing or generating the activity of a phosphate transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a phosphate transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of tyrosine, which comprises
(a) increasing or generating the activity of a ymr013c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of glycine, which comprises
(a) increasing or generating the activity of a purine biosynthesis protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of phenylalanine, which comprises
(a) increasing or generating the activity of a mitochondrial inner membrane protease subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of homoserine, which comprises
(a) increasing or generating the activity of a YNL086W-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

Accordingly, the present invention relates to a process for the production of tyrosine, which comprises
(a) increasing or generating the activity of a amino acid transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of homoserine, which comprises
(a) increasing or generating the activity of a homoserine O-acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

Accordingly, the present invention relates to a process for the production of alanine, which comprises
(a) increasing or generating the activity of a metal ion transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alanine or a composition comprising alanine in said non-human organism or in the culture medium surrounding said non-human organism.

Accordingly, the present invention relates to a process for the production of aspartate, which comprises
(a) increasing or generating the activity of a yol160w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of citrulline, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrulline or a composition comprising citrulline in said non-human organism or in the culture medium surrounding said non-human organism.

Accordingly, the present invention relates to a process for the production of phenylalanine, which comprises
(a) increasing or generating the activity of a histone acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of tyrosine, which comprises
(a) increasing or generating the activity of a histone acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of homoserine, which comprises
(a) increasing or generating the activity of a diacylglycerol O-acyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of homoserine or a composition comprising homoserine in said non-human organism or in the culture medium surrounding said non-human organism.

Accordingly, the present invention relates to a process for the production of serine, which comprises
(a) increasing or generating the activity of a diacylglycerol O-acyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of 5-oxoproline, which comprises
(a) increasing or generating the activity of a diacylglycerol O-acyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of glycine, which comprises
(a) increasing or generating the activity of a acetyl-CoA acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycine or a composition comprising glycine in said non-human organism or in the culture medium surrounding said non-human organism.

Accordingly, the present invention relates to a process for the production of serine, which comprises
(a) increasing or generating the activity of a citrate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of aspartate, which comprises
(a) increasing or generating the activity of a ammonium transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of aspartate or a composition comprising aspartate in said non-human organism or in the culture medium surrounding said non-human organism.

Accordingly, the present invention relates to a process for the production of phenylalanine, which comprises
(a) increasing or generating the activity of a Zm_{—}4842_BE510522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of phenylalanine or a composition comprising phenylalanine in said non-human organism or in the culture medium surrounding said non-human organism.

Accordingly, the present invention relates to a process for the production of serine, which comprises
(a) increasing or generating the activity of a Zm_{—}4842_BE510522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of serine or a composition comprising serine in said non-human organism or in the culture medium surrounding said non-human organism.

Accordingly, the present invention relates to a process for the production of tyrosine, which comprises
(a) increasing or generating the activity of a Zm_{—}4842_BE510522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tyrosine or a composition comprising tyrosine in said non-human organism or in the culture medium surrounding said non-human organism.

Accordingly, the present invention relates to a process for the production of 5-oxoproline, which comprises
(a) increasing or generating the activity of a Zm_{—}4842_BE510522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 5-oxoproline or a composition comprising 5-oxoproline in said non-human organism or in the culture medium surrounding said non-human organism.

Accordingly, the present invention relates to a process for the production of 3,4-dihydroxyphenylalanine (dopa), which comprises
(a) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in the cytosol of a cell of a non-human organism or a part thereof; preferably a microorganism, a plant cell, a plant or a part thereof, as compared to a corresponding non-transformed wild type non-human organism or a part thereof;
(wherein in the respective line in the respective Table in column 7 3,4-dihydroxyphenylalanine (dopa) is depicted) and
(b) growing the non-human organism under conditions which permit the production of 3,4-dihydroxyphenylalanine (dopa), or a composition comprising 3,4-dihydroxyphenylalanine (dopa) in said non-human organism or in the culture medium surrounding said non-human organism.

Accordingly, the present invention relates to a process for the production of 5-oxoproline, which comprises
(a) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in the cytosol of a cell of a non-human organism or a part thereof; preferably a microorganism, a plant cell, a plant or a part thereof, as compared to a corresponding non-transformed wild type non-human organism or a part thereof;
(wherein in the respective line in the respective Table in column 7 5-oxoproline is depicted) and
(b) growing the non-human organism under conditions which permit the production of 5-oxoproline, or a composition comprising 5-oxoproline in said non-human organism or in the culture medium surrounding said non-human organism.

Accordingly, the present invention relates to a process for the production of alanine, which comprises
(a) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in the cytosol of a cell of a non-human organism or a part thereof; preferably a microorganism, a plant cell, a plant or a part thereof, as compared to a corresponding non-transformed wild type non-human organism or a part thereof;
(wherein in the respective line in the respective Table in column 7 alanine is depicted) and
(b) growing the non-human organism under conditions which permit the production of alanine, or a composition comprising alanine in said non-human organism or in the culture medium surrounding said non-human organism.

Accordingly, the present invention relates to a process for the production of asparagine, which comprises
(a) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in the cytosol of a cell of a non-human organism or a part thereof; preferably a microorganism, a plant cell, a plant or a part thereof, as compared to a corresponding non-transformed wild type non-human organism or a part thereof;
(wherein in the respective line in the respective Table in column 7 asparagine is depicted) and
(b) growing the non-human organism under conditions which permit the production of asparagine, or a composition comprising asparagine in said non-human organism or in the culture medium surrounding said non-human organism.

Accordingly, the present invention relates to a process for the production of aspartate, which comprises
(a) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in the cytosol of a cell of a non-human organism or a part thereof; preferably a microorganism, a plant cell, a plant or a part thereof, as compared to a corresponding non-transformed wild type non-human organism or a part thereof;
(wherein in the respective line in the respective Table in column 7 aspartate is depicted) and
(b) growing the non-human organism under conditions which permit the production of aspartate, or a composition comprising aspartate in said non-human organism or in the culture medium surrounding said non-human organism.

Accordingly, the present invention relates to a process for the production of citrulline, which comprises
(a) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in the cytosol of a cell of a non-human organism or a part thereof; preferably a microorganism, a plant cell, a plant or a part thereof, as compared to a corresponding non-transformed wild type non-human organism or a part thereof;
(wherein in the respective line in the respective Table in column 7 citrulline is depicted) and
(b) growing the non-human organism under conditions which permit the production of citrulline, or a composition comprising citrulline in said non-human organism or in the culture medium surrounding said non-human organism.

Accordingly, the present invention relates to a process for the production of glycine, which comprises
(a) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in the cytosol of a cell of a non-human organism or a part thereof; preferably a microorganism, a plant cell, a plant or a part thereof, as compared to a corresponding non-transformed wild type non-human organism or a part thereof;
(wherein in the respective line in the respective Table in column 7 glycine is depicted) and
(b) growing the non-human organism under conditions which permit the production of glycine, or a composition comprising glycine in said non-human organism or in the culture medium surrounding said non-human organism.

Accordingly, the present invention relates to a process for the production of homoserine, which comprises
(a) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in the cytosol of a cell of a non-human organism or a part thereof; preferably a microorganism, a plant cell, a plant or a part thereof, as compared to a corresponding non-transformed wild type non-human organism or a part thereof;
(wherein in the respective line in the respective Table in column 7 homoserine is depicted) and
(b) growing the non-human organism under conditions which permit the production of homoserine, or a composition comprising homoserine in said non-human organism or in the culture medium surrounding said non-human organism.

Accordingly, the present invention relates to a process for the production of ornithine, which comprises
(a) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in the cytosol of a cell of a non-human organism or a part thereof; preferably a microorganism, a plant cell, a plant or a part thereof, as compared to a corresponding non-transformed wild type non-human organism or a part thereof;
(wherein in the respective line in the respective Table in column 7 ornithine is depicted) and
(b) growing the non-human organism under conditions which permit the production of ornithine, or a composition comprising ornithine in said non-human organism or in the culture medium surrounding said non-human organism.

Accordingly, the present invention relates to a process for the production of phenylalanine, which comprises
(a) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in the cytosol of a cell of a non-human organism or a part thereof; preferably a microorganism, a plant cell, a plant or a part thereof, as compared to a corresponding non-transformed wild type non-human organism or a part thereof;
(wherein in the respective line in the respective Table in column 7 phenylalanine is depicted) and
(b) growing the non-human organism under conditions which permit the production of phenylalanine, or a composition comprising phenylalanine in said non-human organism or in the culture medium surrounding said non-human organism.

Accordingly, the present invention relates to a process for the production of serine, which comprises
(a) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in the cytosol of a cell of a non-human organism or a part thereof; preferably a microorganism, a plant cell, a plant or a part thereof, as compared to a corresponding non-transformed wild type non-human organism or a part thereof;
(wherein in the respective line in the respective Table in column 7 serine is depicted) and
(b) growing the non-human organism under conditions which permit the production of serine, or a composition comprising serine in said non-human organism or in the culture medium surrounding said non-human organism.

Accordingly, the present invention relates to a process for the production of tyrosine, which comprises
(a) increasing or generating the activity of a polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3; or
increasing or generating the activity of an expression product of one or more nucleic acid molecule(s) comprising a polynucleotide as depicted in the respective line in column 5 or 8 of Table I, application no. 3, preferably the coding region thereof, or a homolog or a fragment thereof;
in the cytosol of a cell of a non-human organism or a part thereof; preferably a microorganism, a plant cell, a plant or a part thereof, as compared to a corresponding non-transformed wild type non-human organism or a part thereof; (wherein in the respective line in the respective Table in column 7 tyrosine is depicted) and
(b) growing the non-human organism under conditions which permit the production of tyrosine, or a composition comprising tyrosine in said non-human organism or in the culture medium surrounding said non-human organism.

In a further embodiment the activity of the polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3, is increased or generated non-targeted in the above-mentioned process in a microorganism or plant or a part thereof.

In a further embodiment said polypeptide has the activity of the respective polypeptide represented by a protein comprising a polypeptide as depicted in the respective line in column 5 of Table II, application no. 3.

In a further embodiment the activity of the expression product of one or more nucleic acid molecule(s) comprising a polynucleotide as depicted in the respective line in column 5 or 8 of Table I, application no. 3, preferably the coding region thereof, or a homolog or a fragment thereof, is increased or generated non-targeted in the above-mentioned process in a microorganism or plant or a part thereof.

In a further embodiment the activity of the polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3, is increased or generated in the above-mentioned process in an organelle preferably in plastids or mitochondria, especially in plastids, of a microorganism or plant.

In a further embodiment said polypeptide has the activity of the respective polypeptide represented by a protein comprising a polypeptide as depicted in the respective line in column 5 of Table II, application no. 3.

In a further embodiment the activity of the expression product of one or more nucleic acid molecule(s) comprising a polynucleotide as depicted in the respective line in column 5 or 8 of Table I, application no. 3, preferably the coding region thereof, or a homolog or a fragment thereof, is increased or generated in the above-mentioned process in an organelle, preferably in plasids or mitochondria, especially in plastids, of a microorganism or plant.

In a further embodiment the activity of the polypeptide comprising a polypeptide as depicted in the respective line in column 5 or 8 of Table II, application no. 3, or a homolog or a fragment thereof, a consensus sequence or at least one polypeptide motif as depicted in the respective line in column 8 of Table IV, application no. 3, is increased or generated in the above-mentioned process in the cyctosol of a cell, of a microorganism or plant.

In a further embodiment said polypeptide has the activity of the respective polypeptide represented by a protein comprising a polypeptide as depicted in the respective line in column 5 of Table II, application no. 3.

In a further embodiment the activity of the expression product of one or more nucleic acid molecule(s) comprising a polynucleotide as depicted in the respective line in column 5 or 8 of Table I, application no. 3, preferably the coding region thereof, or a homolog or a fragment thereof, is increased or generated in the above-mentioned process in the cytosol of a cell, of a microorganism or plant.

[0039.1.1.3] to [0066.1.1.3] for the disclosure of these paragraphs see [0039.1.1.1] to [0066.1.1.1] above.

As mentioned above the nucleic acid sequences coding for the proteins as shown in Table II, application no. 3, column 5 or 8, or homologs or fragments thereof, are joined to a nucleic acid sequence encoding a plastidic transit peptide, e.g, if for the nucleic acid molecule in Table I, column 6 the term “plastidic” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in Table II, application no. 3, column 5 or 8, or homologs or fragments thereof, e.g., if for the nucleic acid molecule in Table I, column 6 the term “plastidic” is indicated.

0068.1.1.3] to [0072.1.1.3] for the disclosure of these paragraphs see [0068.1.1.1] to [0072.1.1.1] above.

As mentioned above the nucleic acid sequences coding for the proteins as shown in the respective line in Table II, application no. 3, column 5 or 8, or homologs or fragments thereof, are joined to a nucleic acid sequence encoding a mitochondrial transit peptide, e.g., if for the respective nucleic acid molecule in Table I, column 6 the term “mitochondrial” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in the respective line in Table II, application no. 3, column 5 or 8, or homologs or fragments thereof, e.g., if for the nucleic acid molecule in the respective line in Table I, column 6 the term “mitochondrial” is indicated. (For the avoidance of doubt, the term “mitochondric” and “mitochondrial” are interchangeable.)

[0074.1.1.3] to [0075.1.1.3] for the disclosure of these paragraphs see [0074.1.1.1] to [0075.1.1.1] [0075.1.1.1] above.

Nucleic acid sequences coding for the transit peptides may be chemically synthesized either in part or wholly according to structure of transit peptide sequences disclosed in the prior art. Said natural or chemically synthesized sequences can be directly linked to the sequences encoding the mature protein or via a linker nucleic acid sequence, which may be typically less than 500 base pairs, preferably less than 450, 400, 350, 300, 250 or 200 base pairs, more preferably less than 150, 100, 90, 80, 70, 60, 50, 40 or 30 base pairs and most preferably less than 25, 20, 15, 12, 9, 6 or 3 base pairs in length and are in frame to the coding sequence. Furthermore favorable nucleic acid sequences encoding transit peptides may comprise sequences derived from more than one biological and/or chemical source and may include a nucleic acid sequence derived from the amino-terminal region of the mature protein, which in its native state is linked to the transit peptide. In a preferred embodiment of the invention said amino-terminal region of the mature protein is typically less than 150 amino acids, preferably less than 140, 130, 120, 110, 100 or 90 amino acids, more preferably less than 80, 70, 60, 50, 40, 35, 30, 25 or 20 amino acids and most preferably less than 19, 18, 17, 16, 15, 14, 13, 12, 11 or 10 amino acids in length. But even shorter or longer stretches are also possible. In addition target sequences, which facilitate the transport of proteins to other cell compartments such as the vacuole, endoplasmic reticulum, golgi complex, glyoxysomes, peroxisomes, mitochondria (in case of a plastidal transit peptide) or plastids, especially chloroplasts (in case of a mitochondric transit peptide) may be also part of the inventive nucleic acid sequence. The proteins translated from said inventive nucleic acid sequences are a kind of fusion proteins that means the nucleic acid sequences encoding the plastidal transit peptide for example the ones shown in Table a, preferably the last one of the Table a are joint to the respective nucleic acid sequences shown in Table I, application no. 3, columns 5 or 8, especially the coding region thereof, or homologs or fragments thereof, in case of nucleic acid sequences being expressed in plastids, and that means the nucleic acid sequences encoding the mitochondric transit peptide for example the ones shown in Table b, preferably the last one of the Table b are joint to the respective nucleic acid sequences shown in Table I, application no. 3, columns 5 or 8, especially the coding region thereof, or homologs or fragments thereof, in case of nucleic acid sequences being expressed in mitochondria. The person skilled in the art is able to join said sequences in a functional manner. Advantageously the transit peptide part is cleaved off from the protein part shown in Table II, application no. 3, columns 5 or 8, or a homolog or fragment thereof, during or after the transport into the organelles, preferably into the plastids or mitochondria, respectively.

All products of the cleavage of the preferred transit peptide shown in the last line of Table a have preferably the N-terminal amino acid sequences QIA CSS or QIA EFQLTT in front of the start methionine of the respective protein mentioned in Table II, application no. 3, column 5 or 8. or homologs or fragments thereof. Other short amino acid sequences of an range of 1 to 20 amino acids preferable 2 to 15 amino acids, more preferable 3 to 10 amino acids most preferably 4 to 8 amino acids are also possible in front of the start methionine of the protein mentioned in Table II, application no. 3, columns 5 or 8, or homologs or fragments thereof. In case of the amino acid sequence QIA CSS the three amino acids in front of the start methionine are stemming from the LIC (=ligatation independent cloning) cassette. Said short amino acid sequence is preferred in the case of the expression of E. coli genes. In case of the amino acid sequence QIA EFQLTT the six amino acids in front of the start methionine are stemming from the LIC cassette. Said short amino acid sequence is preferred in the case of the expression of S. cerevisiae genes. The skilled worker knowns that other short sequences are also useful in the expression of the respective nucleic acid molecule comprising a polynucleotide as depicted in Table I, application no. 3, column 5 or 8, preferably the coding region thereof, or a homolog or a fragment thereof. Furthermore the skilled worker is aware of the fact that there is not a need for such short sequences in the expression of the genes.

[0077.1.1.3] to [0078.1.1.3] for the disclosure of these paragraphs see [0077.1.1.1] to [0078.1.1.1] above.

Alternatively to the targeting of the respective sequences shown in Table II, application no. 3, columns 5 or 8, or homologs or fragments thereof, preferably of sequences in general encoded in the nucleus with the aid of the targeting sequences mentioned for example in Table a alone, Table b alone, or in combination with other targeting sequences preferably into plastids or mitochondria, respectively, the nucleic acids of the invention can directly be introduced into the plastidal or mitochondrial genome. Therefore in a preferred embodiment the respective nucleic acid sequences shown in the respective line in Table I, application no. 3, columns 5 or 8, preferably the coding region thereof, or homologs or fragments thereof, are directly introduced and expressed in plastids e.g., if for the nucleic acid molecule in the respective line in Table I, column 6, the term “plastidic” is indicated, or are directly introduced and expressed in mitochondria e.g., if for the nucleic acid molecule in the respective line in Table I, column 6 the term “mitochondrial” is indicated.

The term “introduced” in the context of this specification shall mean the insertion of a nucleic acid sequence into the organism by means of a “transfection”, “transduction” or preferably by “transformation”.

A plastid, such as a chloroplast, or a mitochondrion, has been “transformed” by an exogenous (preferably foreign) nucleic acid sequence if a nucleic acid sequence has been introduced into the plastid or mitochondrion that means that this sequence has crossed the membrane or the membranes of the plastid or of the mitochondrion. The exogenous, preferably foreign, DNA may be integrated (covalently linked) into plastid or mitochondrial DNA making up the genome of the plastid or mitochondrion, or it may remain unintegrated (e.g., by including a chloroplast origin of replication). “Stably” integrated DNA sequences are those, which are inherited through plastid or mitochondrion replication, thereby transferring new plastids or mitochondria, with the features of the integrated DNA sequence to the progeny.

[0080.1.1.3] to [0083.1.1.3] for the disclosure of these paragraphs see [0080.1.1.1] to [0083.1.1.1] above.

For the process of the present invention it may be of great advantage that by transforming the plastids the intraspecies specific transgene flow is blocked, because a lot of species such as corn, cotton and rice have a strict maternal inheritance of plastids. By placing the respective genes specified in the respective line in Table I, application no. 3, columns 5 or 8, preferably the coding region, or homologs or fragments thereof, e.g. if for the nucleic acid molecule in the respective line in column 6 of Table I the term “plastidic” is indicated, in the plastids of plants, these genes will not be present in the pollen of said plants.

A further preferred embodiment of the invention relates to the use of so called “chloroplast localization sequences”, in which a first RNA sequence or molecule is capable of transporting or “chaperoning” a second RNA sequence, such as a RNA sequence transcribed from the sequences depicted in the respective line in Table I, application no. 3, columns 5 or 8, preferably the coding region thereof, or a homolog or a fragment thereof, or a sequence encoding a protein, as depicted in the respective line in Table II, application no. 3, columns 5 and 8, or a homolog or a fragment thereof, from an external environment inside a cell or outside a plastid into a chloroplast. In one embodiment the chloroplast localization signal is substantially similar or complementary to a complete or intact viroid sequence. The chloroplast localization signal may be encoded by a DNA sequence, which is transcribed into the chloroplast localization RNA. The term “viroid” refers to a naturally occurring single stranded RNA molecule (Flores, C. R. Acad. Sci. III. 324 (10), 943 (2001)). Viroids usually contain about 200-500 nucleotides and generally exist as circular molecules. Examples of viroids that contain chloroplast localization signals include but are not limited to ASBVd, PLMVd, CChMVd and ELVd. The viroid sequence or a functional part of it can be fused to the sequences depicted in the respective line in Table I, application no. 3, columns 5 or 8, preferably the coding region thereof, or a homolog or a fragment thereof, or a sequence encoding a protein, as depicted in the respective line in Table II, application no. 3, columns 5 or 8, or a homolog or a fragment thereof, in such a manner that the viroid sequence transports a sequence transcribed from a sequence as depicted in the respective line in Table I, application no. 3 columns 5 or 8, preferably the coding region thereof, or a homolog or a fragment thereof, or a sequence encoding a protein as depicted in the respective line in Table II, application no. 3 columns 5 or 8, or a homolog or a fragment thereof, into the chloroplasts, e.g. if for the nucleic acid molecule in column 6 of Table I the term “plastidic” is indicated. A preferred embodiment uses a modified ASBVd (Navarro et al., Virology. 268 (1), 218 (2000)).

In a further specific embodiment the protein to be expressed in the plastids such as the proteins depicted in the respective line in Table II, application no. 3, columns 5 or 8, or a homolog or a fragment thereof, e.g. if for the nucleic acid molecule in the respective line in column 6 of Table I the term “plastidic” is indicated, are encoded by different nucleic acids. Such a method is disclosed in WO 2004/040973, which shall be incorporated by reference. WO 2004/040973 teaches a method, which relates to the translocation of an RNA corresponding to a gene or gene fragment into the chloroplast by means of a chloroplast localization sequence. The genes, which should be expressed in the plant or plant cells, are split into nucleic acid fragments, which are introduced into different compartments in the plant e.g. the nucleus, the plastids and/or mitochondria. Additionally plant cells are described in which the chloroplast contains a ribozyme fused at one end to an RNA encoding a fragment of a protein used in the inventive process such that the ribozyme can trans-splice the translocated fusion RNA to the RNA encoding the gene fragment to form and as the case may be reunite the nucleic acid fragments to an intact mRNA encoding a functional protein for example as disclosed in the respective line in Table II, application no. 3, columns 5 or 8, or a homolog or a fragment thereof.

In a preferred embodiment of the invention the nucleic acid sequences as shown in the respective line in Table I, application no. 3, columns 5 or 8, preferably the coding region thereof, or homologs or fragments thereof, e.g. if for the nucleic acid molecule in the respective line in column 6 of Table I the term “plastidic” is indicated, used in the inventive process are transformed into plastids, which are metabolically active. Those plastids should preferably maintain at a high copy number in the plant or plant tissue of interest, most preferably the chloroplasts found in green plant tissues, such as leaves or cotyledons or in seeds.

For a good expression in the plastids the nucleic acid sequences as shown in the respective line in Table I, application no. 3, columns 5 or 8, preferably the coding region thereof, or homologs or fragments thereof, e.g. if for the nucleic acid molecule in the respective line in column 6 of Table I the term “plastidic” is indicated, are introduced into an expression cassette using preferably a promoter and terminater, which are active in plastids preferably a chloroplast promoter. Examples of such promoters include the psbA promoter from the gene from spinach or pea, the rbcL promoter, and the atpB promoter from corn.

[0089.1.1.3] to [0092.1.1.3] for the disclosure of these paragraphs see [0089.1.1.1] to [0092.1.1.1] above.

Advantageously the process for the production of the fine chemical 3,4-dihydroxyphenylalanine (dopa), 5-oxoproline, alanine, asparagine, aspartate, citrulline, glycine, homoserine, ornithine, phenylalanine, serine, and/or tyrosine leads to an enhanced production of the respective fine chemical. The terms “enhanced” or “increase” mean at least a 10%, 20%, 30%, 40% or 50%, preferably at least 60%, 70%, 80%, 90% or 100%, more preferably 150%, 200%, 300%, 400% or 500% higher production of the respective fine chemical 3,4-dihydroxyphenylalanine (dopa), 5-oxoproline, alanine, asparagine, aspartate, citrulline, glycine, homoserine, ornithine, phenylalanine, serine, and/or tyrosine in comparison to the wild-type as defined above, e.g. that means in comparison to a non-human organism without the aforementioned modification of the activity of a protein as shown in the respective line in Table II, application no. 3, column 5 or 8, or a fragment or a homolog thereof. The modification of the activity of a protein as shown in the respective line in Table II, application no. 3, column 5 or 8, or a homolog or a fragment thereof, or their combination can be achieved by joining the protein to a respective transit peptide, e.g. if for the respective encoding nucleic acid molecule in column 6 of Table I the term “plastidic” or “mitochondrial” is indicated.

Surprisingly it was found, that the transgenic expression of a protein as shown in the respective line in Table II, application no. 3, column 5, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, conferred a production of or an increase in 3,4-dihydroxyphenylalanine (dopa), 5-oxoproline, alanine, asparagine, aspartate, citrulline, glycine, homoserine, ornithine, phenylalanine, serine, and/or tyrosine,respectively, to the transgenic non-human organism as compared to a corresponding non-transformed wild type.

In one embodiment the transgenic expression of a protein as shown in the respective line in Table II, application no. 2, column 5 or 8, or a homolog or a fragment thereof, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, confers a production of or an increase in 3,4-dihydroxyphenylalanine (dopa), 5-oxoproline, alanine, asparagine, aspartate, citrulline, glycine, homoserine, ornithine, phenylalanine, serine, and/or tyrosine, respectively,to the transgenic non-human organism as compared to a corresponding non-transformed wild type.

Accordingly, in one embodiment, an increase of aspartate in a non-human organism, as compared to a corresponding non-transformed wild type non-human organism, is conferred in the process of the invention, if the activity of a polypeptide showing the activity of a 47266012-protein, or if the activity of the polypeptide 47266012_SOYBEAN, preferably represented by SEQ ID NO. 15188, or a homolog or fragment thereof, or if the activity of a polypeptide encoded by a nucleic acid molecule comprising the nucleic acid SEQ ID NO. 15187, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 15187 or polypeptide SEQ ID NO. 15188, respectively, or a homolog or a fragment thereof, is increased or generated, or if the activity 47266012-protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 48 to 125-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a 47266012protein, or if the activity of the polypeptide 47266012_SOYBEAN, preferably represented by SEQ ID NO. 15188, or a homolog or fragment thereof, or if the activity of a polypeptide encoded by a nucleic acid molecule comprising the nucleic acid SEQ ID NO. 15187, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 15187 or polypeptide SEQ ID NO. 15188, respectively, or a homolog or a fragment thereof, is increased or generated, or if the activity 47266012-protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 44 to 122-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a 47266012protein, or if the activity of the polypeptide 47266012_SOYBEAN, preferably represented by SEQ ID NO. 15188, or a homolog or fragment thereof, or if the activity of a polypeptide encoded by a nucleic acid molecule comprising the nucleic acid SEQ ID NO. 15187, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 15187 or polypeptide SEQ ID NO. 15188, respectively, or a homolog or a fragment thereof, is increased or generated, or if the activity 47266012-protein is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 52 to 161-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a 49747384_SOYBEAN-protein, or if the activity of the polypeptide 49747384_SOYBEAN, preferably represented by SEQ ID NO. 70, or a homolog or fragment thereof, or if the activity of a polypeptide encoded by a nucleic acid molecule comprising the nucleic acid SEQ ID NO. 69, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 69 or polypeptide SEQ ID NO. 70, respectively, or a homolog or a fragment thereof, is increased or generated, or if the activity 49747384_SOYBEAN-protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 55 to 122-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a 49747384_SOYBEAN-protein, or if the activity of the polypeptide 49747384_SOYBEAN, preferably represented by SEQ ID NO. 70, or a homolog or fragment thereof, or if the activity of a polypeptide encoded by a nucleic acid molecule comprising the nucleic acid SEQ ID NO. 69, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 69 or polypeptide SEQ ID NO. 70, respectively, or a homolog or a fragment thereof, is increased or generated, or if the activity 49747384_SOYBEAN-protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 35 to 80-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a 49747384_SOYBEAN-protein, or if the activity of the polypeptide 49747384_SOYBEAN, preferably represented by SEQ ID NO. 70, or a homolog or fragment thereof, or if the activity of a polypeptide encoded by a nucleic acid molecule comprising the nucleic acid SEQ ID NO. 69, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 69 or polypeptide SEQ ID NO. 70, respectively, or a homolog or a fragment thereof, is increased or generated, or if the activity 49747384_SOYBEAN-protein is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 52 to 101 percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a pyruvate kinase, or if the activity of the polypeptide 51340801_CANOLA, preferably represented by SEQ ID NO. 15533, or a homolog or fragment thereof, or if the activity of a polypeptide encoded by a nucleic acid molecule comprising the nucleic acid SEQ ID NO. 15532, preferably the coding region thereof, or a homolog or fragment thereof, e.g. derived from Brassica napus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 15532 or polypeptide SEQ ID NO. 15533, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line discloses in column 7 alanine. For example, an increase of the alanine of at least 1 percent, particularly in a range of 38 to 153-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a pyruvate kinase, or if the activity of the polypeptide 51340801_CANOLA, preferably represented by SEQ ID NO. 15533, or a homolog or fragment thereof, or if the activity of a polypeptide encoded by a nucleic acid molecule comprising the nucleic acid SEQ ID NO. 15532, preferably the coding region thereof, or a homolog or fragment thereof, e.g. derived from Brassica napus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 15532 or polypeptide SEQ ID NO. 15533, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line discloses in column 7 phenylalanine. For example, an increase of the phenylalanine of at least 1 percent, particularly in a range of 30 to 263-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a pyruvate kinase, or if the activity of the polypeptide 51340801_CANOLA, preferably represented by SEQ ID NO. 15533, or a homolog or fragment thereof, or if the activity of a polypeptide encoded by a nucleic acid molecule comprising the nucleic acid SEQ ID NO. 15532, preferably the coding region thereof, or a homolog or fragment thereof, e.g. derived from Brassica napus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 15532 or polypeptide SEQ ID NO. 15533, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line discloses in column 7 tyrosine. For example, an increase of the tyrosine of at least 1 percent, particularly in a range of 42 to 123-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a acetyltransferase, or if the activity of the polypeptide 59547452_SOYBEAN, preferably represented by SEQ ID NO. 16156, or a homolog or fragment thereof, or if the activity of a polypeptide encoded by a nucleic acid molecule comprising the nucleic acid SEQ ID NO. 16155, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 16155 or polypeptide SEQ ID NO. 16156, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line discloses in column 7 citrulline. For example, an increase of the citrulline of at least 1 percent, particularly in a range of 40 to 104-percent is conferred as compared to a corresponding non-transformed wild type non-human organism.

Accordingly, in one embodiment, an increase of asparagine in a non-human organism, as compared to a corresponding non-transformed wild type non-human organism, is conferred in the process of the invention, if the activity of a polypeptide showing the activity of a pyruvate kinase, or if the activity of the polypeptide 59554615_SOYBEAN, preferably represented by SEQ ID NO. 16264, or a homolog or fragment thereof, or if the activity of a polypeptide encoded by a nucleic acid molecule comprising the nucleic acid SEQ ID NO. 16263, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 16263 or polypeptide SEQ ID NO. 16264, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line discloses in column 7 asparagine. For example, an increase of the asparagine of at least 1 percent, particularly in a range of 54 to 113-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a pyruvate kinase, or if the activity of the polypeptide 59554615_SOYBEAN, preferably represented by SEQ ID NO. 16264, or a homolog or fragment thereof, or if the activity of a polypeptide encoded by a nucleic acid molecule comprising the nucleic acid SEQ ID NO. 16263, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 16263 or polypeptide SEQ ID NO. 16264, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line discloses in column 7 serine. For example, an increase of the serine of at least 1 percent, particularly in a range of 27 to 31-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a glucose-6-phosphate 1-dehydrogenase, or if the activity of the polypeptide 59582753_SOYBEAN, preferably represented by SEQ ID NO. 16884, or a homolog or fragment thereof, or if the activity of a polypeptide encoded by a nucleic acid molecule comprising the nucleic acid SEQ ID NO. 16883, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No.

3, column 5 or 8 in the respective same line as the nucleic acid molecule SEQ ID NO. 16883 or polypeptide SEQ ID NO. 16884, respectively, or a homolog or a fragment thereof, is increased or generated, or if the activity glucose-6-phosphate 1-dehydrogenase is increased or generated in a non-human organism, like a microorganism or a plant cell, plant or part thereof, especially with plastidic localization, whereby the respective line discloses in column 7 alanine. For example, an increase of the alanine of at least 1 percent, particularly in a range of 23 to 116-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a glucose-6-phosphate 1-dehydrogenase, or if the activity of the polypeptide 59582753_SOYBEAN, preferably represented by SEQ ID NO. 16884, or a homolog or fragment thereof, or if the activity of a polypeptide encoded by a nucleic acid molecule comprising the nucleic acid SEQ ID NO. 16883, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 16883 or polypeptide SEQ ID NO. 16884, respectively, or a homolog or a fragment thereof, is increased or generated, or if the activity glucose-6-phosphate 1-dehydrogenase is increased or generated in a non-human organism, like a microorganism or a plant cell, plant or part thereof, especially with plastidic localization, whereby the respective line discloses in column 7 phenylalanine. For example, an increase of the phenylalanine of at least 1 percent, particularly in a range of 27 to 61-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a glucose-6-phosphate 1-dehydrogenase, or if the activity of the polypeptide 59582753_SOYBEAN, preferably represented by SEQ ID NO. 16884, or a homolog or fragment thereof, or if the activity of a polypeptide encoded by a nucleic acid molecule comprising the nucleic acid SEQ ID NO. 16883, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No.

3, column 5 or 8 in the respective same line as the nucleic acid molecule SEQ ID NO. 16883 or polypeptide SEQ ID NO. 16884, respectively, or a homolog or a fragment thereof, is increased or generated, or if the activity glucose-6-phosphate 1-dehydrogenase is increased or generated in a non-human organism, like a microorganism or a plant cell, plant or part thereof, especially with plastidic localization, whereby the respective line discloses in column 7 tyrosine. For example, an increase of the tyrosine of at least 1 percent, particularly in a range of 38 to 93-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a amino acid acetyltransferase, or if the activity of the polypeptide AAC43185, preferably represented by SEQ ID NO. 17357, or a homolog or fragment thereof, or if the activity of a polypeptide encoded by a nucleic acid molecule comprising the nucleic acid SEQ ID NO. 17356, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 17356 or polypeptide SEQ ID NO. 17357, respectively, or a homolog or a fragment thereof, is increased or generated, or if the activity amino acid acetyltransferase is increased or generated in a non-human organism, like a microorganism or a plant cell, plant or part thereof, especially with plastidic localization, whereby the respective line discloses in column 7 alanine. For example, an increase of the alanine of at least 1 percent, particularly in a range of 22 to 31-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a amino acid acetyltransferase, or if the activity of the polypeptide AAC43185, preferably represented by SEQ ID NO. 17357, or a homolog or fragment thereof, or if the activity of a polypeptide encoded by a nucleic acid molecule comprising the nucleic acid SEQ ID NO. 17356, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 17356 or polypeptide SEQ ID NO. 17357, respectively, or a homolog or a fragment thereof, is increased or generated, or if the activity amino acid acetyltransferase is increased or generated in a non-human organism, like a microorganism or a plant cell, plant or part thereof, especially with plastidic localization, whereby the respective line discloses in column 7 citrulline. For example, an increase of the citrulline of at least 1 percent, particularly in a range of 76 to 618-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a amino acid acetyltransferase, or if the activity of the polypeptide AAC43185, preferably represented by SEQ ID NO. 17357, or a homolog or fragment thereof, or if the activity of a polypeptide encoded by a nucleic acid molecule comprising the nucleic acid SEQ ID NO. 17356, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 17356 or polypeptide SEQ ID NO. 17357, respectively, or a homolog or a fragment thereof, is increased or generated, or if the activity amino acid acetyltransferase is increased or generated in a non-human organism, like a microorganism or a plant cell, plant or part thereof, especially with plastidic localization, whereby the respective line discloses in column 7 phenylalanine. For example, an increase of the phenylalanine of at least 1 percent, particularly in a range of 26 to 55-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a protein phosphatase, or if the activity of the polypeptide At1g07430, preferably represented by SEQ ID NO. 17452, or a homolog or fragment thereof, or if the activity of a polypeptide encoded by a nucleic acid molecule comprising the nucleic acid SEQ ID NO. 17451, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 17451 or polypeptide SEQ ID NO. 17452, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 citrulline. For example, an increase of the citrulline of at least 1 percent, particularly in a range of 34 to 55-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a DNA-binding protein, or if the activity of the polypeptide At1g14490, preferably represented by SEQ ID NO. 386, or a homolog or fragment thereof, or if the activity of a polypeptide encoded by a nucleic acid molecule comprising the nucleic acid SEQ ID NO. 385, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 385 or polypeptide SEQ ID NO. 386, respectively, or a homolog or a fragment thereof, is increased or generated, or if the activity DNA-binding protein is increased or generated in a non-human organism, like a microorganism or a plant cell, plant or part thereof, especially with non-targeted localization, whereby the respective line discloses in column 7 glycine. For example, an increase of the glycine of at least 1 percent, particularly in a range of 97 to 147-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a DNA-binding protein, or if the activity of the polypeptide At1g14490, preferably represented by SEQ ID NO. 386, or a homolog or fragment thereof, or if the activity of a polypeptide encoded by a nucleic acid molecule comprising the nucleic acid SEQ ID NO. 385, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 385 or polypeptide SEQ ID NO. 386, respectively, or a homolog or a fragment thereof, is increased or generated, or if the activity DNA-binding protein is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 27 to 51-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a At1g17440protein, or if the activity of the polypeptide At1g17440, preferably represented by SEQ ID NO. 17602, or a homolog or fragment thereof, or if the activity of a polypeptide encoded by a nucleic acid molecule comprising the nucleic acid SEQ ID NO. 17601, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 17601 or polypeptide SEQ ID NO. 17602, respectively, or a homolog or a fragment thereof, is increased or generated, or if the activity At1g17440-protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 33 to 54-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a At1g17440protein, or if the activity of the polypeptide At1g17440, preferably represented by SEQ ID NO. 17602, or a homolog or fragment thereof, or if the activity of a polypeptide encoded by a nucleic acid molecule comprising the nucleic acid SEQ ID NO. 17601, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 17601 or polypeptide SEQ ID NO. 17602, respectively, or a homolog or a fragment thereof, is increased or generated, or if the activity At1g17440-protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 48 to 106-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a At1g19800protein, or if the activity of the polypeptide At1g19800, preferably represented by SEQ ID NO. 17638, or a homolog or fragment thereof, or if the activity of a polypeptide encoded by a nucleic acid molecule comprising the nucleic acid SEQ ID NO. 17637, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 17637 or polypeptide SEQ ID NO. 17638, respectively, or a homolog or a fragment thereof, is increased or generated, or if the activity At1g19800-protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 49 to 86-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a At1g19800protein, or if the activity of the polypeptide At1g19800, preferably represented by SEQ ID NO. 17638, or a homolog or fragment thereof, or if the activity of a polypeptide encoded by a nucleic acid molecule comprising the nucleic acid SEQ ID NO. 17637, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 17637 or polypeptide SEQ ID NO. 17638, respectively, or a homolog or a fragment thereof, is increased or generated, or if the activity At1g19800-protein is increased or generated in a non-human organism, like a microorganism or a plant cell, plant or part thereof, especially with non-targeted localization, whereby the respective line discloses in column 7 citrulline. For example, an increase of the citrulline of at least 1 percent, particularly in a range of 35 to 66-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a At1g19800 protein, or if the activity of the polypeptide At1g19800, preferably represented by SEQ ID NO. 17638, or a homolog or fragment thereof, or if the activity of a polypeptide encoded by a nucleic acid molecule comprising the nucleic acid SEQ ID NO. 17637, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 17637 or polypeptide SEQ ID NO. 17638, respectively, or a homolog or a fragment thereof, is increased or generated, or if the activity At1g19800-protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 41 to 398-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a cullin, or if the activity of the polypeptide At1g26830, preferably represented by SEQ ID NO. 17702, or a homolog or fragment thereof, or if the activity of a polypeptide encoded by a nucleic acid molecule comprising the nucleic acid SEQ ID NO. 17701, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 17701 or polypeptide SEQ ID NO. 17702, respectively, or a homolog or a fragment thereof, is increased or generated, or if the activity cullin is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 35 to 134-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a cullin, or if the activity of the polypeptide At1g26830, preferably represented by SEQ ID NO. 17702, or a homolog or fragment thereof, or if the activity of a polypeptide encoded by a nucleic acid molecule comprising the nucleic acid SEQ ID NO. 17701, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 17701 or polypeptide SEQ ID NO. 17702, respectively, or a homolog or a fragment thereof, is increased or generated, or if the activity cullin is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 39 to 89-percent is conferred as compared to a corresponding non-transformed wild type non-human organism.

Accordingly, in one embodiment, an increase of homoserine in a non-human organism, as compared to a corresponding non-transformed wild type non-human organism, is conferred in the process of the invention, if the activity of a polypeptide showing the activity of a bifunctional aspartokinase/homoserine dehydrogenase, or if the activity of the polypeptide At1 g31230, preferably represented by SEQ ID NO. 628, or a homolog or fragment thereof, or if the activity of a polypeptide encoded by a nucleic acid molecule comprising the nucleic acid SEQ ID NO. 627, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 627 or polypeptide SEQ ID NO. 628, respectively, or a homolog or a fragment thereof, is increased or generated, or if the activity bifunctional aspartokinase/homoserine dehydrogenase is increased or generated in a non-human organism, like a microorganism or a plant cell, plant or part thereof, especially with non-targeted localization, whereby the respective line discloses in column 7 homoserine. For example, an increase of the homoserine of at least 1 percent, particularly in a range of 84 to 1108-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a eukaryotic translation initiation factor 5, or if the activity of the polypeptide At1 g36730, preferably represented by SEQ ID NO. 17969, or a homolog or fragment thereof, or if the activity of a polypeptide encoded by a nucleic acid molecule comprising the nucleic acid SEQ ID NO. 17968, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 17968 or polypeptide SEQ ID NO. 17969, respectively, or a homolog or a fragment thereof, is increased or generated, or if the activity eukaryotic translation initiation factor 5 is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 51 to 87-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a eukaryotic translation initiation factor 5, or if the activity of the polypeptide At1g36730, preferably represented by SEQ ID NO. 17969, or a homolog or fragment thereof, or if the activity of a polypeptide encoded by a nucleic acid molecule comprising the nucleic acid SEQ ID NO. 17968, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 17968 or polypeptide SEQ ID NO. 17969, respectively, or a homolog or a fragment thereof, is increased or generated, or if the activity eukaryotic translation initiation factor 5 is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 32 to 97-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a eukaryotic translation initiation factor 5, or if the activity of the polypeptide At1 g36730, preferably represented by SEQ ID NO. 17969, or a homolog or fragment thereof, or if the activity of a polypeptide encoded by a nucleic acid molecule comprising the nucleic acid SEQ ID NO. 17968, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 17968 or polypeptide SEQ ID NO. 17969, respectively, or a homolog or a fragment thereof, is increased or generated, or if the activity eukaryotic translation initiation factor 5 is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 25 to 72-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a eukaryotic translation initiation factor 5, or if the activity of the polypeptide At1 g36730, preferably represented by SEQ ID NO. 17969, or a homolog or fragment thereof, or if the activity of a polypeptide encoded by a nucleic acid molecule comprising the nucleic acid SEQ ID NO. 17968, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 17968 or polypeptide SEQ ID NO. 17969, respectively, or a homolog or a fragment thereof, is increased or generated, or if the activity eukaryotic translation initiation factor 5 is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 36 to 86-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a eukaryotic translation initiation factor 5, or if the activity of the polypeptide At1 g36730, preferably represented by SEQ ID NO. 17969, or a homolog or fragment thereof, or if the activity of a polypeptide encoded by a nucleic acid molecule comprising the nucleic acid SEQ ID NO. 17968, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 17968 or polypeptide SEQ ID NO. 17969, respectively, or a homolog or a fragment thereof, is increased or generated, or if the activity eukaryotic translation initiation factor 5 is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 39 to 74-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a transcriptional regulator, or if the activity of the polypeptide At1g43850, preferably represented by SEQ ID NO. 18071, or a homolog or fragment thereof, or if the activity of a polypeptide encoded by a nucleic acid molecule comprising the nucleic acid SEQ ID NO. 18070, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 18070 or polypeptide SEQ ID NO. 18071, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 77 to 358-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a transcriptional regulator, or if the activity of the polypeptide At1g43850, preferably represented by SEQ ID NO. 18071, or a homolog or fragment thereof, or if the activity of a polypeptide encoded by a nucleic acid molecule comprising the nucleic acid SEQ ID NO. 18070, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 18070 or polypeptide SEQ ID NO. 18071, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 70 to 205-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a transcriptional regulator, or if the activity of the polypeptide At1 g43850, preferably represented by SEQ ID NO. 18071, or a homolog or fragment thereof, or if the activity of a polypeptide encoded by a nucleic acid molecule comprising the nucleic acid SEQ ID NO. 18070, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 18070 or polypeptide SEQ ID NO. 18071, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 76 to 313-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a transcriptional regulator, or if the activity of the polypeptide At1 g43850, preferably represented by SEQ ID NO. 18071, or a homolog or fragment thereof, or if the activity of a polypeptide encoded by a nucleic acid molecule comprising the nucleic acid SEQ ID NO. 18070, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 18070 or polypeptide SEQ ID NO. 18071, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 41 to 210-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a protein phosphatase, or if the activity of the polypeptide At1g48040, preferably represented by SEQ ID NO. 813, or a homolog or fragment thereof, or if the activity of a polypeptide encoded by a nucleic acid molecule comprising the nucleic acid SEQ ID NO. 812, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 812 or polypeptide SEQ ID NO. 813, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 glycine. For example, an increase of the glycine of at least 1 percent, particularly in a range of 36 to 52-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a protein kinase, or if the activity of the polypeptide At1g48260, preferably represented by SEQ ID NO. 18236, or a homolog or fragment thereof, or if the activity of a polypeptide encoded by a nucleic acid molecule comprising the nucleic acid SEQ ID NO. 18235, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 18235 or polypeptide SEQ ID NO. 18236, respectively, or a homolog or a fragment thereof, is increased or generated, or if the activity protein kinase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 58 to 432-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a protein kinase, or if the activity of the polypeptide At1g48260, preferably represented by SEQ ID NO. 18236, or a homolog or fragment thereof, or if the activity of a polypeptide encoded by a nucleic acid molecule comprising the nucleic acid SEQ ID NO. 18235, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 18235 or polypeptide SEQ ID NO. 18236, respectively, or a homolog or a fragment thereof, is increased or generated, or if the activity protein kinase is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 29 to 154-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a protein kinase, or if the activity of the polypeptide At1g48260, preferably represented by SEQ ID NO. 18236, or a homolog or fragment thereof, or if the activity of a polypeptide encoded by a nucleic acid molecule comprising the nucleic acid SEQ ID NO. 18235, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 18235 or polypeptide SEQ ID NO. 18236, respectively, or a homolog or a fragment thereof, is increased or generated, or if the activity protein kinase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 48 to 182-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a calcium-dependent protein kinase, or if the activity of the polypeptide At1g61950, preferably represented by SEQ ID NO. 18870, or a homolog or fragment thereof, or if the activity of a polypeptide encoded by a nucleic acid molecule comprising the nucleic acid SEQ ID NO. 18869, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 18869 or polypeptide SEQ ID NO. 18870, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 60 to 312-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a calcium-dependent protein kinase, or if the activity of the polypeptide At1g61950, preferably represented by SEQ ID NO. 18870, or a homolog or fragment thereof, or if the activity of a polypeptide encoded by a nucleic acid molecule comprising the nucleic acid SEQ ID NO. 18869, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 18869 or polypeptide SEQ ID NO. 18870, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 65 to 149-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a protein phosphatase, or if the activity of the polypeptide At1g72770, preferably represented by SEQ ID NO. 19420, or a homolog or fragment thereof, or if the activity of a polypeptide encoded by a nucleic acid molecule comprising the nucleic acid SEQ ID NO. 19419, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 19419 or polypeptide SEQ ID NO. 19420, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 26 to 121-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a protein phosphatase, or if the activity of the polypeptide At1g72770, preferably represented by SEQ ID NO. 19420, or a homolog or fragment thereof, or if the activity of a polypeptide encoded by a nucleic acid molecule comprising the nucleic acid SEQ ID NO. 19419, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 19419 or polypeptide SEQ ID NO. 19420, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 glycine. For example, an increase of the glycine of at least 1 percent, particularly in a range of 48 to 108-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a protein phosphatase, or if the activity of the polypeptide At1g72770, preferably represented by SEQ ID NO. 19420, or a homolog or fragment thereof, or if the activity of a polypeptide encoded by a nucleic acid molecule comprising the nucleic acid SEQ ID NO. 19419, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 19419 or polypeptide SEQ ID NO. 19420, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 35 to 150-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a protein phosphatase, or if the activity of the polypeptide At1g72770, preferably represented by SEQ ID NO. 19420, or a homolog or fragment thereof, or if the activity of a polypeptide encoded by a nucleic acid molecule comprising the nucleic acid SEQ ID NO. 19419, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 19419 or polypeptide SEQ ID NO. 19420, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 45 to 171-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a aminotransferase, or if the activity of the polypeptide At2g13360, preferably represented by SEQ ID NO. 68537, or a homolog or fragment thereof, or if the activity of a polypeptide encoded by a nucleic acid molecule comprising the nucleic acid SEQ ID NO. 68536, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 68536 or polypeptide SEQ ID NO. 68537, respectively, or a homolog or a fragment thereof, is increased or generated, or if the activity aminotransferase is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 23 to 44-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a transcription factor, or if the activity of the polypeptide At2g17560, preferably represented by SEQ ID NO. 19503, or a homolog or fragment thereof, or if the activity of a polypeptide encoded by a nucleic acid molecule comprising the nucleic acid SEQ ID NO. 19502, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 19502 or polypeptide SEQ ID NO. 19503, respectively, or a homolog or a fragment thereof, is increased or generated, or if the activity transcription factor is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 32 to 315-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a transcription factor, or if the activity of the polypeptide At2g17560, preferably represented by SEQ ID NO. 19503, or a homolog or fragment thereof, or if the activity of a polypeptide encoded by a nucleic acid molecule comprising the nucleic acid SEQ ID NO. 19502, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 19502 or polypeptide SEQ ID NO. 19503, respectively, or a homolog or a fragment thereof, is increased or generated, or if the activity transcription factor is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 37 to 182-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a protein phosphatase, or if the activity of the polypeptide At2g25070, preferably represented by SEQ ID NO. 1299, or a homolog or fragment thereof, or if the activity of a polypeptide encoded by a nucleic acid molecule comprising the nucleic acid SEQ ID NO. 1298, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 1298 or polypeptide SEQ ID NO. 1299, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 74 to 147-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a protein phosphatase, or if the activity of the polypeptide At2g25070, preferably represented by SEQ ID NO. 1299, or a homolog or fragment thereof, or if the activity of a polypeptide encoded by a nucleic acid molecule comprising the nucleic acid SEQ ID NO. 1298, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 1298 or polypeptide SEQ ID NO. 1299, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 38 to 78-percent is conferred as compared to a corresponding non-transformed wild type non-human organism.

Accordingly, in one embodiment, an increase of asparagine in a non-human organism, as compared to a corresponding non-transformed wild type non-human organism, is conferred in the process of the invention, if the activity of a polypeptide showing the activity of a protein phosphatase, or if the activity of the polypeptide At2g28890, preferably represented by SEQ ID NO. 19875, or a homolog or fragment thereof, or if the activity of a polypeptide encoded by a nucleic acid molecule comprising the nucleic acid SEQ ID NO. 19874, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 19874 or polypeptide SEQ ID NO. 19875, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 asparagine. For example, an increase of the asparagine of at least 1 percent, particularly in a range of 57 to 929-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a protein phosphatase, or if the activity of the polypeptide At2g28890, preferably represented by SEQ ID NO. 19875, or a homolog or fragment thereof, or if the activity of a polypeptide encoded by a nucleic acid molecule comprising the nucleic acid SEQ ID NO. 19874, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 19874 or polypeptide SEQ ID NO. 19875, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 36 to 520-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a protein phosphatase, or if the activity of the polypeptide At2g28890, preferably represented by SEQ ID NO. 19875, or a homolog or fragment thereof, or if the activity of a polypeptide encoded by a nucleic acid molecule comprising the nucleic acid SEQ ID NO. 19874, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 19874 or polypeptide SEQ ID NO. 19875, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 29 to 164-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a protein phosphatase, or if the activity of the polypeptide At2g28890, preferably represented by SEQ ID NO. 19875, or a homolog or fragment thereof, or if the activity of a polypeptide encoded by a nucleic acid molecule comprising the nucleic acid SEQ ID NO. 19874, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 19874 or polypeptide SEQ ID NO. 19875, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 62 to 555-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a CBL-interacting protein kinase, or if the activity of the polypeptide At2g30360, preferably represented by SEQ ID NO. 19920, or a homolog or fragment thereof, or if the activity of a polypeptide encoded by a nucleic acid molecule comprising the nucleic acid SEQ ID NO. 19919, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 19919 or polypeptide SEQ ID NO. 19920, respectively, or a homolog or a fragment thereof, is increased or generated, or if the activity CBL-interacting protein kinase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 29 to 548-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a CBL-interacting protein kinase, or if the activity of the polypeptide At2g30360, preferably represented by SEQ ID NO. 19920, or a homolog or fragment thereof, or if the activity of a polypeptide encoded by a nucleic acid molecule comprising the nucleic acid SEQ ID NO. 19919, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 19919 or polypeptide SEQ ID NO. 19920, respectively, or a homolog or a fragment thereof, is increased or generated, or if the activity CBL-interacting protein kinase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 38 to 323-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a CBL-interacting protein kinase, or if the activity of the polypeptide At2g30360, preferably represented by SEQ ID NO. 19920, or a homolog or fragment thereof, or if the activity of a polypeptide encoded by a nucleic acid molecule comprising the nucleic acid SEQ ID NO. 19919, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 19919 or polypeptide SEQ ID NO. 19920, respectively, or a homolog or a fragment thereof, is increased or generated, or if the activity CBL-interacting protein kinase is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 33 to 92-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a glutaredoxin, or if the activity of the polypeptide At2g30540, preferably represented by SEQ ID NO. 20347, or a homolog or fragment thereof, or if the activity of a polypeptide encoded by a nucleic acid molecule comprising the nucleic acid SEQ ID NO. 20346, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 20346 or polypeptide SEQ ID NO. 20347, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 24 to 171-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a glutaredoxin, or if the activity of the polypeptide At2g30540, preferably represented by SEQ ID NO. 20347, or a homolog or fragment thereof, or if the activity of a polypeptide encoded by a nucleic acid molecule comprising the nucleic acid SEQ ID NO. 20346, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 20346 or polypeptide SEQ ID NO. 20347, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 43 to 106-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a CBL-interacting protein kinase, or if the activity of the polypeptide At2g34180, preferably represented by SEQ ID NO. 20579, or a homolog or fragment thereof, or if the activity of a polypeptide encoded by a nucleic acid molecule comprising the nucleic acid SEQ ID NO. 20578, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 20578 or polypeptide SEQ ID NO. 20579, respectively, or a homolog or a fragment thereof, is increased or generated, or if the activity CBL-interacting protein kinase is increased or generated in a non-human organism, like a microorganism or a plant cell, plant or part thereof, especially with non-targeted localization, whereby the respective line discloses in column 7 glycine. For example, an increase of the glycine of at least 1 percent, particularly in a range of 56 to 191-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a delta-1-pyrroline 5-carboxylase synthetase, or if the activity of the polypeptide At2g39800, preferably represented by SEQ ID NO. 21009, or a homolog or fragment thereof, or if the activity of a polypeptide encoded by a nucleic acid molecule comprising the nucleic acid SEQ ID NO. 21008, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 21008 or polypeptide SEQ ID NO. 21009, respectively, or a homolog or a fragment thereof, is increased or generated, or if the activity delta-1-pyrroline 5-carboxylase synthetase is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 20 to 34 percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a delta-1-pyrroline 5-carboxylase synthetase, or if the activity of the polypeptide At2g39800, preferably represented by SEQ ID NO. 21009, or a homolog or fragment thereof, or if the activity of a polypeptide encoded by a nucleic acid molecule comprising the nucleic acid SEQ ID NO. 21008, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 21008 or polypeptide SEQ ID NO. 21009, respectively, or a homolog or a fragment thereof, is increased or generated, or if the activity delta-1-pyrroline 5-carboxylase synthetase is increased or generated in a non-human organism, like a microorganism or a plant cell, plant or part thereof, especially with non-targeted localization, whereby the respective line discloses in column 7 glycine. For example, an increase of the glycine of at least 1 percent, particularly in a range of 34 to 83 percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a glutaredoxin, or if the activity of the polypeptide At2g47880, preferably represented by SEQ ID NO. 21160, or a homolog or fragment thereof, or if the activity of a polypeptide encoded by a nucleic acid molecule comprising the nucleic acid SEQ ID NO. 21159, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 21159 or polypeptide SEQ ID NO. 21160, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 64 to 241-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a glutaredoxin, or if the activity of the polypeptide At2g47880, preferably represented by SEQ ID NO. 21160, or a homolog or fragment thereof, or if the activity of a polypeptide encoded by a nucleic acid molecule comprising the nucleic acid SEQ ID NO. 21159, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 21159 or polypeptide SEQ ID NO. 21160, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 62 to 219-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a heat shock transcription factor, or if the activity of the polypeptide At3g02990, preferably represented by SEQ ID NO. 68658, or a homolog or fragment thereof, or if the activity of a polypeptide encoded by a nucleic acid molecule comprising the nucleic acid SEQ ID NO. 68657, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 68657 or polypeptide SEQ ID NO. 68658, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 46 to 84-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a pyruvate kinase, or if the activity of the polypeptide At3g04050, preferably represented by SEQ ID NO. 21498, or a homolog or fragment thereof, or if the activity of a polypeptide encoded by a nucleic acid molecule comprising the nucleic acid SEQ ID NO. 21497, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 21497 or polypeptide SEQ ID NO. 21498, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line discloses in column 7 alanine. For example, an increase of the alanine of at least 1 percent, particularly in a range of 30 to 950-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a pyruvate kinase, or if the activity of the polypeptide At3g04050, preferably represented by SEQ ID NO. 21498, or a homolog or fragment thereof, or if the activity of a polypeptide encoded by a nucleic acid molecule comprising the nucleic acid SEQ ID NO. 21497, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 21497 or polypeptide SEQ ID NO. 21498, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line discloses in column 7 phenylalanine. For example, an increase of the phenylalanine of at least 1 percent, particularly in a range of 104 to 300-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a pyruvate kinase, or if the activity of the polypeptide At3g04050, preferably represented by SEQ ID NO. 21498, or a homolog or fragment thereof, or if the activity of a polypeptide encoded by a nucleic acid molecule comprising the nucleic acid SEQ ID NO. 21497, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 21497 or polypeptide SEQ ID NO. 21498, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line discloses in column 7 tyrosine. For example, an increase of the tyrosine of at least 1 percent, particularly in a range of 41 to 346-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a ankyrin repeat family protein, or if the activity of the polypeptide At3g04710, preferably represented by SEQ ID NO. 21903, or a homolog or fragment thereof, or if the activity of a polypeptide encoded by a nucleic acid molecule comprising the nucleic acid SEQ ID NO. 21902, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 21902 or polypeptide SEQ ID NO. 21903, respectively, or a homolog or a fragment thereof, is increased or generated, or if the activity ankyrin repeat family protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 50 to 58-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a protein phosphatase, or if the activity of the polypeptide At3g06270, preferably represented by SEQ ID NO. 22016, or a homolog or fragment thereof, or if the activity of a polypeptide encoded by a nucleic acid molecule comprising the nucleic acid SEQ ID NO. 22015, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 22015 or polypeptide SEQ ID NO. 22016, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 44 to 46-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a thioredoxin family protein, or if the activity of the polypeptide At3g08710, preferably represented by SEQ ID NO. 22250, or a homolog or fragment thereof, or if the activity of a polypeptide encoded by a nucleic acid molecule comprising the nucleic acid SEQ ID NO. 22249, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 22249 or polypeptide SEQ ID NO. 22250, respectively, or a homolog or a fragment thereof, is increased or generated, or if the activity thioredoxin family protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 77 to 149-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a thioredoxin family protein, or if the activity of the polypeptide At3g08710, preferably represented by SEQ ID NO. 22250, or a homolog or fragment thereof, or if the activity of a polypeptide encoded by a nucleic acid molecule comprising the nucleic acid SEQ ID NO. 22249, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 22249 or polypeptide SEQ ID NO. 22250, respectively, or a homolog or a fragment thereof, is increased or generated, or if the activity thioredoxin family protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 57 to 205-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a thioredoxin family protein, or if the activity of the polypeptide At3g08710, preferably represented by SEQ ID NO. 22250, or a homolog or fragment thereof, or if the activity of a polypeptide encoded by a nucleic acid molecule comprising the nucleic acid SEQ ID NO. 22249, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 22249 or polypeptide SEQ ID NO. 22250, respectively, or a homolog or a fragment thereof, is increased or generated, or if the activity thioredoxin family protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 50 to 132-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a harpin-induced family protein, or if the activity of the polypeptide At3g11650, preferably represented by SEQ ID NO. 22612, or a homolog or fragment thereof, or if the activity of a polypeptide encoded by a nucleic acid molecule comprising the nucleic acid SEQ ID NO. 22611, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 22611 or polypeptide SEQ ID NO. 22612, respectively, or a homolog or a fragment thereof, is increased or generated, or if the activity harpin-induced family protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 34 to 149-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a harpin-induced family protein, or if the activity of the polypeptide At3g11650, preferably represented by SEQ ID NO. 22612, or a homolog or fragment thereof, or if the activity of a polypeptide encoded by a nucleic acid molecule comprising the nucleic acid SEQ ID NO. 22611, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 22611 or polypeptide SEQ ID NO. 22612, respectively, or a homolog or a fragment thereof, is increased or generated, or if the activity harpin-induced family protein is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 24 to 89-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a harpin-induced family protein, or if the activity of the polypeptide At3g11650, preferably represented by SEQ ID NO. 22612, or a homolog or fragment thereof, or if the activity of a polypeptide encoded by a nucleic acid molecule comprising the nucleic acid SEQ ID NO. 22611, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 22611 or polypeptide SEQ ID NO. 22612, respectively, or a homolog or a fragment thereof, is increased or generated, or if the activity harpin-induced family protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 35 to 125-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a DNA mismatch repair protein, or if the activity of the polypeptide At3g18524, preferably represented by SEQ ID NO. 22833, or a homolog or fragment thereof, or if the activity of a polypeptide encoded by a nucleic acid molecule comprising the nucleic acid SEQ ID NO. 22832, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 22832 or polypeptide SEQ ID NO. 22833, respectively, or a homolog or a fragment thereof, is increased or generated, or if the activity DNA mismatch repair protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 30 to 48-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a protein kinase, or if the activity of the polypeptide At3g23000, preferably represented by SEQ ID NO. 1816, or a homolog or fragment thereof, or if the activity of a polypeptide encoded by a nucleic acid molecule comprising the nucleic acid SEQ ID NO. 1815, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 1815 or polypeptide SEQ ID NO. 1816, respectively, or a homolog or a fragment thereof, is increased or generated, or if the activity protein kinase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 75 to 455-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a protein kinase, or if the activity of the polypeptide At3g23000, preferably represented by SEQ ID NO. 1816, or a homolog or fragment thereof, or if the activity of a polypeptide encoded by a nucleic acid molecule comprising the nucleic acid SEQ ID NO. 1815, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 1815 or polypeptide SEQ ID NO. 1816, respectively, or a homolog or a fragment thereof, is increased or generated, or if the activity protein kinase is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 23 to 78-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a protein kinase, or if the activity of the polypeptide At3g23000, preferably represented by SEQ ID NO. 1816, or a homolog or fragment thereof, or if the activity of a polypeptide encoded by a nucleic acid molecule comprising the nucleic acid SEQ ID NO. 1815, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 1815 or polypeptide SEQ ID NO. 1816, respectively, or a homolog or a fragment thereof, is increased or generated, or if the activity protein kinase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 67 to 183-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a protein kinase, or if the activity of the polypeptide At3g23000, preferably represented by SEQ ID NO. 1816, or a homolog or fragment thereof, or if the activity of a polypeptide encoded by a nucleic acid molecule comprising the nucleic acid SEQ ID NO. 1815, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 1815 or polypeptide SEQ ID NO. 1816, respectively, or a homolog or a fragment thereof, is increased or generated, or if the activity protein kinase is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 42 to 110-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a eukaryotic translation initiation factor, or if the activity of the polypeptide At3g26400, preferably represented by SEQ ID NO. 68728, or a homolog or fragment thereof, or if the activity of a polypeptide encoded by a nucleic acid molecule comprising the nucleic acid SEQ ID NO. 68727, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 68727 or polypeptide SEQ ID NO. 68728, respectively, or a homolog or a fragment thereof, is increased or generated, or if the activity eukaryotic translation initiation factor is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 23 to 62-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a glucose-6-phosphate 1-dehydrogenase, or if the activity of the polypeptide At3g27300, preferably represented by SEQ ID NO. 23003, or a homolog or fragment thereof, or if the activity of a polypeptide encoded by a nucleic acid molecule comprising the nucleic acid SEQ ID NO. 23002, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 23002 or polypeptide SEQ ID NO. 23003, respectively, or a homolog or a fragment thereof, is increased or generated, or if the activity glucose-6-phosphate 1-dehydrogenase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 37 to 87-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a glycosyl transferase, or if the activity of the polypeptide At3g27540, preferably represented by SEQ ID NO. 68778, or a homolog or fragment thereof, or if the activity of a polypeptide encoded by a nucleic acid molecule comprising the nucleic acid SEQ ID NO. 68777, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 68777 or polypeptide SEQ ID NO. 68778, respectively, or a homolog or a fragment thereof, is increased or generated, or if the activity glycosyl transferase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 36 to 179-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a glycosyl transferase, or if the activity of the polypeptide At3g27540, preferably represented by SEQ ID NO. 68778, or a homolog or fragment thereof, or if the activity of a polypeptide encoded by a nucleic acid molecule comprising the nucleic acid SEQ ID NO. 68777, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 68777 or polypeptide SEQ ID NO. 68778, respectively, or a homolog or a fragment thereof, is increased or generated, or if the activity glycosyl transferase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 42 to 324-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a auxin response factor, or if the activity of the polypeptide At3g61830, preferably represented by SEQ ID NO. 2368, or a homolog or fragment thereof, or if the activity of a polypeptide encoded by a nucleic acid molecule comprising the nucleic acid SEQ ID NO. 2367, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 2367 or polypeptide SEQ ID NO. 2368, respectively, or a homolog or a fragment thereof, is increased or generated, or if the activity auxin response factor is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 58 to 540-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a auxin response factor, or if the activity of the polypeptide At3g61830, preferably represented by SEQ ID NO. 2368, or a homolog or fragment thereof, or if the activity of a polypeptide encoded by a nucleic acid molecule comprising the nucleic acid SEQ ID NO. 2367, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 2367 or polypeptide SEQ ID NO. 2368, respectively, or a homolog or a fragment thereof, is increased or generated, or if the activity auxin response factor is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 36 to 784-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a monthiol glutaredoxin, or if the activity of the polypeptide At3g62930, preferably represented by SEQ ID NO. 2574, or a homolog or fragment thereof, or if the activity of a polypeptide encoded by a nucleic acid molecule comprising the nucleic acid SEQ ID NO. 2573, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 2573 or polypeptide SEQ ID NO. 2574, respectively, or a homolog or a fragment thereof, is increased or generated, or if the activity monthiol glutaredoxin is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 48 to 117-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a monthiol glutaredoxin, or if the activity of the polypeptide At3g62930, preferably represented by SEQ ID NO. 2574, or a homolog or fragment thereof, or if the activity of a polypeptide encoded by a nucleic acid molecule comprising the nucleic acid SEQ ID NO. 2573, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 2573 or polypeptide SEQ ID NO. 2574, respectively, or a homolog or a fragment thereof, is increased or generated, or if the activity monthiol glutaredoxin is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 26 to 274-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 105 to 324-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 55 to 249-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a glutare doxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 37 to 235-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 33 to 139-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 36 to 211-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a monothiol glutaredoxin, or if the activity of the polypeptide At4g15670, preferably represented by SEQ ID NO. 3280, or a homolog or fragment thereof, or if the activity of a polypeptide encoded by a nucleic acid molecule comprising the nucleic acid SEQ ID NO. 3279, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 3279 or polypeptide SEQ ID NO. 3280, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 58 to 140-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a monothiol glutaredoxin, or if the activity of the polypeptide At4g15690, preferably represented by SEQ ID NO. 23845, or a homolog or fragment thereof, or if the activity of a polypeptide encoded by a nucleic acid molecule comprising the nucleic acid SEQ ID NO. 23844, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 23844 or polypeptide SEQ ID NO. 23845, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 75 to 169-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a monothiol glutaredoxin, or if the activity of the polypeptide At4g15690, preferably represented by SEQ ID NO. 23845, or a homolog or fragment thereof, or if the activity of a polypeptide encoded by a nucleic acid molecule comprising the nucleic acid SEQ ID NO. 23844, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 23844 or polypeptide SEQ ID NO. 23845, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 29 to 90-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a monothiol glutaredoxin, or if the activity of the polypeptide At4g15690, preferably represented by SEQ ID NO. 23845, or a homolog or fragment thereof, or if the activity of a polypeptide encoded by a nucleic acid molecule comprising the nucleic acid SEQ ID NO. 23844, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 23844 or polypeptide SEQ ID NO. 23845, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 41 to 150-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a monothiol glutaredoxin, or if the activity of the polypeptide At4g15690, preferably represented by SEQ ID NO. 23845, or a homolog or fragment thereof, or if the activity of a polypeptide encoded by a nucleic acid molecule comprising the nucleic acid SEQ ID NO. 23844, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 23844 or polypeptide SEQ ID NO. 23845, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 36 to 106-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a monothiol glutaredoxin, or if the activity of the polypeptide At4g15700, preferably represented by SEQ ID NO. 3655, or a homolog or fragment thereof, or if the activity of a polypeptide encoded by a nucleic acid molecule comprising the nucleic acid SEQ ID NO. 3654, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 3654 or polypeptide SEQ ID NO. 3655, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 52 to 121-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a heat shock transcription factor, or if the activity of the polypeptide At4g18880, preferably represented by SEQ ID NO. 24233, or a homolog or fragment thereof, or if the activity of a polypeptide encoded by a nucleic acid molecule comprising the nucleic acid SEQ ID NO. 24232, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 24232 or polypeptide SEQ ID NO. 24233, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 23 to 140-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a protein phosphatase, or if the activity of the polypeptide At4g26080, preferably represented by SEQ ID NO. 68850, or a homolog or fragment thereof, or if the activity of a polypeptide encoded by a nucleic acid molecule comprising the nucleic acid SEQ ID NO. 68849, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 68849 or polypeptide SEQ ID NO. 68850, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 24 to 81-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a protein phosphatase, or if the activity of the polypeptide At4g26080, preferably represented by SEQ ID NO. 68850, or a homolog or fragment thereof, or if the activity of a polypeptide encoded by a nucleic acid molecule comprising the nucleic acid SEQ ID NO. 68849, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 68849 or polypeptide SEQ ID NO. 68850, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 54 to 123-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a protein phosphatase, or if the activity of the polypeptide At4g26080, preferably represented by SEQ ID NO. 68850, or a homolog or fragment thereof, or if the activity of a polypeptide encoded by a nucleic acid molecule comprising the nucleic acid SEQ ID NO. 68849, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 68849 or polypeptide SEQ ID NO. 68850, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 citrulline. For example, an increase of the citrulline of at least 1 percent, particularly in a range of 42 to 59-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a At4g32480-protein, or if the activity of the polypeptide At4g32480, preferably represented by SEQ ID NO. 4041, or a homolog or fragment thereof, or if the activity of a polypeptide encoded by a nucleic acid molecule comprising the nucleic acid SEQ ID NO. 4040, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 4040 or polypeptide SEQ ID NO. 4041, respectively, or a homolog or a fragment thereof, is increased or generated, or if the activity At4g32480-protein is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 48 to 318-percent is conferred as compared to a corresponding non-transformed wild type non-human organism.

Accordingly, in one embodiment, an increase of asparagine in a non-human organism, as compared to a corresponding non-transformed wild type non-human organism, is conferred in the process of the invention, if the activity of a polypeptide showing the activity of a At4g32480protein, or if the activity of the polypeptide At4g32480, preferably represented by SEQ ID NO. 4041, or a homolog or fragment thereof, or if the activity of a polypeptide encoded by a nucleic acid molecule comprising the nucleic acid SEQ ID NO. 4040, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 4040 or polypeptide SEQ ID NO. 4041, respectively, or a homolog or a fragment thereof, is increased or generated, or if the activity At4g32480-protein is increased or generated in a non-human organism, like a microorganism or a plant cell, plant or part thereof, especially with non-targeted localization, whereby the respective line discloses in column 7 asparagine. For example, an increase of the asparagine of at least 1 percent, particularly in a range of 86 to 1206-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a At4g32480-protein, or if the activity of the polypeptide At4g32480, preferably represented by SEQ ID NO. 4041, or a homolog or fragment thereof, or if the activity of a polypeptide encoded by a nucleic acid molecule comprising the nucleic acid SEQ ID NO. 4040, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 4040 or polypeptide SEQ ID NO. 4041, respectively, or a homolog or a fragment thereof, is increased or generated, or if the activity At4g32480-protein is increased or generated in a non-human organism, like a microorganism or a plant cell, plant or part thereof, especially with non-targeted localization, whereby the respective line discloses in column 7 glycine. For example, an increase of the glycine of at least 1 percent, particularly in a range of 38 to 686-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a At4g32480protein, or if the activity of the polypeptide At4g32480, preferably represented by SEQ ID NO. 4041, or a homolog or fragment thereof, or if the activity of a polypeptide encoded by a nucleic acid molecule comprising the nucleic acid SEQ ID NO. 4040, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 4040 or polypeptide SEQ ID NO. 4041, respectively, or a homolog or a fragment thereof, is increased or generated, or if the activity At4g32480-protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 31 to 410-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a At4g32480-protein, or if the activity of the polypeptide At4g32480, preferably represented by SEQ ID NO. 4041, or a homolog or fragment thereof, or if the activity of a polypeptide encoded by a nucleic acid molecule comprising the nucleic acid SEQ ID NO. 4040, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 4040 or polypeptide SEQ ID NO. 4041, respectively, or a homolog or a fragment thereof, is increased or generated, or if the activity At4g32480-protein is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 40 to 98-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a At4g32480-protein, or if the activity of the polypeptide At4g32480, preferably represented by SEQ ID NO. 4041, or a homolog or fragment thereof, or if the activity of a polypeptide encoded by a nucleic acid molecule comprising the nucleic acid SEQ ID NO. 4040, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 4040 or polypeptide SEQ ID NO. 4041, respectively, or a homolog or a fragment thereof, is increased or generated, or if the activity At4g32480-protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 54 to 437-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a glutaredoxin, or if the activity of the polypeptide At4g33040, preferably represented by SEQ ID NO. 4103, or a homolog or fragment thereof, or if the activity of a polypeptide encoded by a nucleic acid molecule comprising the nucleic acid SEQ ID NO. 4102, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 4102 or polypeptide SEQ ID NO. 4103, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 48 to 194-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a glutaredoxin, or if the activity of the polypeptide At4g33040, preferably represented by SEQ ID NO. 4103, or a homolog or fragment thereof, or if the activity of a polypeptide encoded by a nucleic acid molecule comprising the nucleic acid SEQ ID NO. 4102, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 4102 or polypeptide SEQ ID NO. 4103, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 citrulline. For example, an increase of the citrulline of at least 1 percent, particularly in a range of 30 to 52-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a cyclin D , or if the activity of the polypeptide At4g34160, preferably represented by SEQ ID NO. 24312, or a homolog or fragment thereof, or if the activity of a polypeptide encoded by a nucleic acid molecule comprising the nucleic acid SEQ ID NO. 24311, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 24311 or polypeptide SEQ ID NO. 24312, respectively, or a homolog or a fragment thereof, is increased or generated, or if the activity cyclin D is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 29 to 152-percent is conferred as compared to a corresponding non-transformed wild type non-human organism.

Accordingly, in one embodiment, an increase of asparagine in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line discloses in column 7 asparagine. For example, an increase of the asparagine of at least 1 percent, particularly in a range of 52 to 443-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 191 to 534-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 67 to 140-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 149 to 427-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 99 to 175-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a protein phosphatase, or if the activity of the polypeptide At5g02760, preferably represented by SEQ ID NO. 68988, or a homolog or fragment thereof, or if the activity of a polypeptide encoded by a nucleic acid molecule comprising the nucleic acid SEQ ID NO. 68987, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 68987 or polypeptide SEQ ID NO. 68988, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 54 to 95-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a heat shock transcription factor, or if the activity of the polypeptide At5g03720, preferably represented by SEQ ID NO. 24439, or a homolog or fragment thereof, or if the activity of a polypeptide encoded by a nucleic acid molecule comprising the nucleic acid SEQ ID NO. 24438, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 24438 or polypeptide SEQ ID NO. 24439, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 56 to 212-percent is conferred as compared to a corresponding non-transformed wild type non-human organism.

Accordingly, in one embodiment, an increase of 3,4-dihydroxyphenylalanine (dopa) in a non-human organism, as compared to a corresponding non-transformed wild type non-human organism, is conferred in the process of the invention, if the activity of a polypeptide showing the activity of a heat shock transcription factor, or if the activity of the polypeptide At5g03720, preferably represented by SEQ ID NO. 24439, or a homolog or fragment thereof, or if the activity of a polypeptide encoded by a nucleic acid molecule comprising the nucleic acid SEQ ID NO. 24438, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 24438 or polypeptide SEQ ID NO. 24439, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line discloses in column 7 3,4-dihydroxyphenylalanine (dopa). For example, an increase of the 3,4-dihydroxyphenylalanine (dopa) of at least 1 percent, particularly in a range of 48 to 124-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a gibberellin 20-oxidase, or if the activity of the polypeptide At5g07200, preferably represented by SEQ ID NO. 24493, or a homolog or fragment thereof, or if the activity of a polypeptide encoded by a nucleic acid molecule comprising the nucleic acid SEQ ID NO. 24492, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 24492 or polypeptide SEQ ID NO. 24493, respectively, or a homolog or a fragment thereof, is increased or generated, or if the activity gibberellin 20-oxidase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 30 to 100-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a gibberellin 20-oxidase, or if the activity of the polypeptide At5g07200, preferably represented by SEQ ID NO. 24493, or a homolog or fragment thereof, or if the activity of a polypeptide encoded by a nucleic acid molecule comprising the nucleic acid SEQ ID NO. 24492, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 24492 or polypeptide SEQ ID NO. 24493, respectively, or a homolog or a fragment thereof, is increased or generated, or if the activity gibberellin 20-oxidase is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 24 to 98-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a integral membrane transporter family protein, or if the activity of the polypeptide At5g10820, preferably represented by SEQ ID NO. 25223, or a homolog or fragment thereof, or if the activity of a polypeptide encoded by a nucleic acid molecule comprising the nucleic acid SEQ ID NO. 25222, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 25222 or polypeptide SEQ ID NO. 25223, respectively, or a homolog or a fragment thereof, is increased or generated, or if the activity integral membrane transporter family protein is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 26 to 72-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a glutaredoxin, or if the activity of the polypeptide At5g14070, preferably represented by SEQ ID NO. 69276, or a homolog or fragment thereof, or if the activity of a polypeptide encoded by a nucleic acid molecule comprising the nucleic acid SEQ ID NO. 69275, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 69275 or polypeptide SEQ ID NO. 69276, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 27 to 127-percent is conferred as compared to a corresponding non-transformed wild type non-human organism.

Accordingly, in one embodiment, an increase of asparagine in a non-human organism, as compared to a corresponding non-transformed wild type non-human organism, is conferred in the process of the invention, if the activity of a polypeptide showing the activity of a At5g16650-protein, or if the activity of the polypeptide At5g16650, preferably represented by SEQ ID NO. 25284, or a homolog or fragment thereof, or if the activity of a polypeptide encoded by a nucleic acid molecule comprising the nucleic acid SEQ ID NO. 25283, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 25283 or polypeptide SEQ ID NO. 25284, respectively, or a homolog or a fragment thereof, is increased or generated, or if the activity At5g16650-protein is increased or generated in a non-human organism, like a microorganism or a plant cell, plant or part thereof, especially with non-targeted localization, whereby the respective line discloses in column 7 asparagine. For example, an increase of the asparagine of at least 1 percent, particularly in a range of 48 to 386-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a At5g16650-protein, or if the activity of the polypeptide At5g16650, preferably represented by SEQ ID NO. 25284, or a homolog or fragment thereof, or if the activity of a polypeptide encoded by a nucleic acid molecule comprising the nucleic acid SEQ ID NO. 25283, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 25283 or polypeptide SEQ ID NO. 25284, respectively, or a homolog or a fragment thereof, is increased or generated, or if the activity At5g16650-protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 48 to 224-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a At5g16650-protein, or if the activity of the polypeptide At5g16650, preferably represented by SEQ ID NO. 25284, or a homolog or fragment thereof, or if the activity of a polypeptide encoded by a nucleic acid molecule comprising the nucleic acid SEQ ID NO. 25283, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 25283 or polypeptide SEQ ID NO. 25284, respectively, or a homolog or a fragment thereof, is increased or generated, or if the activity At5g16650-protein is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 35 to 139-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a At5g16650-protein, or if the activity of the polypeptide At5g16650, preferably represented by SEQ ID NO. 25284, or a homolog or fragment thereof, or if the activity of a polypeptide encoded by a nucleic acid molecule comprising the nucleic acid SEQ ID NO. 25283, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 25283 or polypeptide SEQ ID NO. 25284, respectively, or a homolog or a fragment thereof, is increased or generated, or if the activity At5g16650-protein is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 45 to 158-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a monothiol glutaredoxin, or if the activity of the polypeptide At5g18600, preferably represented by SEQ ID NO. 4905, or a homolog or fragment thereof, or if the activity of a polypeptide encoded by a nucleic acid molecule comprising the nucleic acid SEQ ID NO. 4904, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 4904 or polypeptide SEQ ID NO. 4905, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 43 to 184-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a monothiol glutaredoxin, or if the activity of the polypeptide At5g18600, preferably represented by SEQ ID NO. 4905, or a homolog or fragment thereof, or if the activity of a polypeptide encoded by a nucleic acid molecule comprising the nucleic acid SEQ ID NO. 4904, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 4904 or polypeptide SEQ ID NO. 4905, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 66 to 212-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a at5g21910protein, or if the activity of the polypeptide At5g21910, preferably represented by SEQ ID NO. 69554, or a homolog or fragment thereof, or if the activity of a polypeptide encoded by a nucleic acid molecule comprising the nucleic acid SEQ ID NO. 69553, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 69553 or polypeptide SEQ ID NO. 69554, respectively, or a homolog or a fragment thereof, is increased or generated, or if the activity at5g21910-protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 29 to 67-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a transcription factor, or if the activity of the polypeptide At5g39760, preferably represented by SEQ ID NO. 25429, or a homolog or fragment thereof, or if the activity of a polypeptide encoded by a nucleic acid molecule comprising the nucleic acid SEQ ID NO. 25428, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 25428 or polypeptide SEQ ID NO. 25429, respectively, or a homolog or a fragment thereof, is increased or generated, or if the activity transcription factor is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 53 to 87-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a thioredoxin, or if the activity of the polypeptide At5g39950, preferably represented by SEQ ID NO. 69575, or a homolog or fragment thereof, or if the activity of a polypeptide encoded by a nucleic acid molecule comprising the nucleic acid SEQ ID NO. 69574, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 69574 or polypeptide SEQ ID NO. 69575, respectively, or a homolog or a fragment thereof, is increased or generated, or if the activity thioredoxin is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 25 to 57-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a protein phosphatase, or if the activity of the polypeptide At5g57050, preferably represented by SEQ ID NO. 5319, or a homolog or fragment thereof, or if the activity of a polypeptide encoded by a nucleic acid molecule comprising the nucleic acid SEQ ID NO. 5318, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 5318 or polypeptide SEQ ID NO. 5319, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 31 to 116-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a protein phosphatase, or if the activity of the polypeptide At5g57050, preferably represented by SEQ ID NO. 5319, or a homolog or fragment thereof, or if the activity of a polypeptide encoded by a nucleic acid molecule comprising the nucleic acid SEQ ID NO. 5318, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 5318 or polypeptide SEQ ID NO. 5319, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 citrulline. For example, an increase of the citrulline of at least 1 percent, particularly in a range of 42 to 54-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a protein phosphatase, or if the activity of the polypeptide At5g59220, preferably represented by SEQ ID NO. 25499, or a homolog or fragment thereof, or if the activity of a polypeptide encoded by a nucleic acid molecule comprising the nucleic acid SEQ ID NO. 25498, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 25498 or polypeptide SEQ ID NO. 25499, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 citrulline. For example, an increase of the citrulline of at least 1 percent, particularly in a range of 41 to 122-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a RNA-binding protein, or if the activity of the polypeptide At5g60110, preferably represented by SEQ ID NO. 70007, or a homolog or fragment thereof, or if the activity of a polypeptide encoded by a nucleic acid molecule comprising the nucleic acid SEQ ID NO. 70006, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 70006 or polypeptide SEQ ID NO. 70007, respectively, or a homolog or a fragment thereof, is increased or generated, or if the activity RNA-binding protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 34 to 70-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a RNA-binding protein, or if the activity of the polypeptide At5g60110, preferably represented by SEQ ID NO. 70007, or a homolog or fragment thereof, or if the activity of a polypeptide encoded by a nucleic acid molecule comprising the nucleic acid SEQ ID NO. 70006, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 70006 or polypeptide SEQ ID NO. 70007, respectively, or a homolog or a fragment thereof, is increased or generated, or if the activity RNA-binding protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 47 to 170-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a pyruvate kinase, or if the activity of the polypeptide At5g63680, preferably represented by SEQ ID NO. 70039, or a homolog or fragment thereof, or if the activity of a polypeptide encoded by a nucleic acid molecule comprising the nucleic acid SEQ ID NO. 70038, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 70038 or polypeptide SEQ ID NO. 70039, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line discloses in column 7 alanine. For example, an increase of the alanine of at least 1 percent, particularly in a range of 42 to 264-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a zinc finger protein, or if the activity of the polypeptide At5g64920, preferably represented by SEQ ID NO. 5494, or a homolog or fragment thereof, or if the activity of a polypeptide encoded by a nucleic acid molecule comprising the nucleic acid SEQ ID NO. 5493, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 5493 or polypeptide SEQ ID NO. 5494, respectively, or a homolog or a fragment thereof, is increased or generated, or if the activity zinc finger protein is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 22 to 41-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a zinc finger protein, or if the activity of the polypeptide At5g64920, preferably represented by SEQ ID NO. 5494, or a homolog or fragment thereof, or if the activity of a polypeptide encoded by a nucleic acid molecule comprising the nucleic acid SEQ ID NO. 5493, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 5493 or polypeptide SEQ ID NO. 5494, respectively, or a homolog or a fragment thereof, is increased or generated, or if the activity zinc finger protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 27 to 106-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a zinc finger protein, or if the activity of the polypeptide At5g64920, preferably represented by SEQ ID NO. 5494, or a homolog or fragment thereof, or if the activity of a polypeptide encoded by a nucleic acid molecule comprising the nucleic acid SEQ ID NO. 5493, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 5493 or polypeptide SEQ ID NO. 5494, respectively, or a homolog or a fragment thereof, is increased or generated, or if the activity zinc finger protein is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 29 to 82-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a zinc finger protein, or if the activity of the polypeptide At5g64920, preferably represented by SEQ ID NO. 5494, or a homolog or fragment thereof, or if the activity of a polypeptide encoded by a nucleic acid molecule comprising the nucleic acid SEQ ID NO. 5493, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 5493 or polypeptide SEQ ID NO. 5494, respectively, or a homolog or a fragment thereof, is increased or generated, or if the activity zinc finger protein is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 44 to 151-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a CDP-diacylglycerol-serine O-phosphatidyltransferase, or if the activity of the polypeptide AvinDRAFT_{—}0035, preferably represented by SEQ ID NO. 70584, or a homolog or fragment thereof, or if the activity of a polypeptide encoded by a nucleic acid molecule comprising the nucleic acid SEQ ID NO. 70583, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 70583 or polypeptide SEQ ID NO. 70584, respectively, or a homolog or a fragment thereof, is increased or generated, or if the activity CDP-diacylglycerol-serine O-phosphatidyltransferase is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 53 to 163-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a CDP-diacylglycerol-serine O-phosphatidyltransferase, or if the activity of the polypeptide AvinDRAFT_{—}0035, preferably represented by SEQ ID NO. 70584, or a homolog or fragment thereof, or if the activity of a polypeptide encoded by a nucleic acid molecule comprising the nucleic acid SEQ ID NO. 70583, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 70583 or polypeptide SEQ ID NO. 70584, respectively, or a homolog or a fragment thereof, is increased or generated, or if the activity CDP-diacylglycerol-serine O-phosphatidyltransferase is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 48 to 123-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a Glycine cleavage system T aminomethyltransferase, or if the activity of the polypeptide AVINDRAFT_{—}0539, preferably represented by SEQ ID NO. 70716, or a homolog or fragment thereof, or if the activity of a polypeptide encoded by a nucleic acid molecule comprising the nucleic acid SEQ ID NO. 70715, preferably the coding region thereof, or a homolog or fragment thereof, e.g. derived from AZOTOBACTER VINELANDII, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 70715 or polypeptide SEQ ID NO. 70716, respectively, or a homolog or a fragment thereof, is increased or generated, or if the activity Glycine cleavage system T aminomethyltransferase is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 30 to 52-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a transcriptional regulator, or if the activity of the polypeptide AvinDRAFT_{—}1045, preferably represented by SEQ ID NO. 25677, or a homolog or fragment thereof, or if the activity of a polypeptide encoded by a nucleic acid molecule comprising the nucleic acid SEQ ID NO. 25676, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 25676 or polypeptide SEQ ID NO. 25677, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 26 to 49-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a adenylylsulfate kinase, or if the activity of the polypeptide AvinDRAFT_{—}1398, preferably represented by SEQ ID NO. 25781, or a homolog or fragment thereof, or if the activity of a polypeptide encoded by a nucleic acid molecule comprising the nucleic acid SEQ ID NO. 25780, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 25780 or polypeptide SEQ ID NO. 25781, respectively, or a homolog or a fragment thereof, is increased or generated, or if the activity adenylylsulfate kinase is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 49 to 101-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a malic enzyme, or if the activity of the polypeptide AvinDRAFT_{—}1495, preferably represented by SEQ ID NO. 5558, or a homolog or fragment thereof, or if the activity of a polypeptide encoded by a nucleic acid molecule comprising the nucleic acid SEQ ID NO. 5557, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 5557 or polypeptide SEQ ID NO. 5558, respectively, or a homolog or a fragment thereof, is increased or generated, or if the activity malic enzyme is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 24 to 45-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a malic enzyme, or if the activity of the polypeptide AvinDRAFT_{—}1495, preferably represented by SEQ ID NO. 5558, or a homolog or fragment thereof, or if the activity of a polypeptide encoded by a nucleic acid molecule comprising the nucleic acid SEQ ID NO. 5557, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 5557 or polypeptide SEQ ID NO. 5558, respectively, or a homolog or a fragment thereof, is increased or generated, or if the activity malic enzyme is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 25 to 197-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a malic enzyme, or if the activity of the polypeptide AvinDRAFT_{—}1495, preferably represented by SEQ ID NO. 5558, or a homolog or fragment thereof, or if the activity of a polypeptide encoded by a nucleic acid molecule comprising the nucleic acid SEQ ID NO. 5557, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 5557 or polypeptide SEQ ID NO. 5558, respectively, or a homolog or a fragment thereof, is increased or generated, or if the activity malic enzyme is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 35 to 357-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a sec-independent protein translocase, or if the activity of the polypeptide AvinDRAFT_{—}1624, preferably represented by SEQ ID NO. 26197, or a homolog or fragment thereof, or if the activity of a polypeptide encoded by a nucleic acid molecule comprising the nucleic acid SEQ ID NO. 26196, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 26196 or polypeptide SEQ ID NO. 26197, respectively, or a homolog or a fragment thereof, is increased or generated, or if the activity sec-independent protein translocase is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 20 to 30-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a pyruvate kinase, or if the activity of the polypeptide AvinDRAFT_{—}1806, preferably represented by SEQ ID NO. 26435, or a homolog or fragment thereof, or if the activity of a polypeptide encoded by a nucleic acid molecule comprising the nucleic acid SEQ ID NO. 26434, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 26434 or polypeptide SEQ ID NO. 26435, respectively, or a homolog or a fragment thereof, is increased or generated, or if the activity pyruvate kinase is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 50 to 127-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a betahydroxylase, or if the activity of the polypeptide AvinDRAFT_{—}2091, preferably represented by SEQ ID NO. 6041, or a homolog or fragment thereof, or if the activity of a polypeptide encoded by a nucleic acid molecule comprising the nucleic acid SEQ ID NO. 6040, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 6040 or polypeptide SEQ ID NO. 6041, respectively, or a homolog or a fragment thereof, is increased or generated, or if the activity beta-hydroxylase is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 24 to 36-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a betahydroxylase, or if the activity of the polypeptide AvinDRAFT_{—}2091, preferably represented by SEQ ID NO. 6041, or a homolog or fragment thereof, or if the activity of a polypeptide encoded by a nucleic acid molecule comprising the nucleic acid SEQ ID NO. 6040, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 6040 or polypeptide SEQ ID NO. 6041, respectively, or a homolog or a fragment thereof, is increased or generated, or if the activity beta-hydroxylase is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 89 to 237-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a beta-hydroxylase, or if the activity of the polypeptide AvinDRAFT_{—}2091, preferably represented by SEQ ID NO. 6041, or a homolog or fragment thereof, or if the activity of a polypeptide encoded by a nucleic acid molecule comprising the nucleic acid SEQ ID NO. 6040, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 6040 or polypeptide SEQ ID NO. 6041, respectively, or a homolog or a fragment thereof, is increased or generated, or if the activity beta-hydroxylase is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 47 to 75-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a elongation factor Tu, or if the activity of the polypeptide AvinDRAFT_{—}2344, preferably represented by SEQ ID NO. 27022, or a homolog or fragment thereof, or if the activity of a polypeptide encoded by a nucleic acid molecule comprising the nucleic acid SEQ ID NO. 27021, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 27021 or polypeptide SEQ ID NO. 27022, respectively, or a homolog or a fragment thereof, is increased or generated, or if the activity elongation factor Tu is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 83 to 94-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a elongation factor Tu, or if the activity of the polypeptide AvinDRAFT_{—}2344, preferably represented by SEQ ID NO. 27022, or a homolog or fragment thereof, or if the activity of a polypeptide encoded by a nucleic acid molecule comprising the nucleic acid SEQ ID NO. 27021, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 27021 or polypeptide SEQ ID NO. 27022, respectively, or a homolog or a fragment thereof, is increased or generated, or if the activity elongation factor Tu is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 57 to 254-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a elongation factor Tu, or if the activity of the polypeptide AvinDRAFT_{—}2344, preferably represented by SEQ ID NO. 27022, or a homolog or fragment thereof, or if the activity of a polypeptide encoded by a nucleic acid molecule comprising the nucleic acid SEQ ID NO. 27021, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 27021 or polypeptide SEQ ID NO. 27022, respectively, or a homolog or a fragment thereof, is increased or generated, or if the activity elongation factor Tu is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 32 to 198-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a elongation facfor Tu, or if the activity of the polypeptide AvinDRAFT_{—}2344, preferably represented by SEQ ID NO. 27022, or a homolog or fragment thereof, or if the activity of a polypeptide encoded by a nucleic acid molecule comprising the nucleic acid SEQ ID NO. 27021, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 27021 or polypeptide SEQ ID NO. 27022, respectively, or a homolog or a fragment thereof, is increased or generated, or if the activity elongation factor Tu is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 42 to 298-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a elongation factor Tu, or if the activity of the polypeptide AvinDRAFT_{—}2344, preferably represented by SEQ ID NO. 27022, or a homolog or fragment thereof, or if the activity of a polypeptide encoded by a nucleic acid molecule comprising the nucleic acid SEQ ID NO. 27021, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 27021 or polypeptide SEQ ID NO. 27022, respectively, or a homolog or a fragment thereof, is increased or generated, or if the activity elongation factor Tu is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 50 to 194-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a 30S ribosomal protein S3, or if the activity of the polypeptide AvinDRAFT_{—}2365, preferably represented by SEQ ID NO. 71326, or a homolog or fragment thereof, or if the activity of a polypeptide encoded by a nucleic acid molecule comprising the nucleic acid SEQ ID NO. 71325, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 71325 or polypeptide SEQ ID NO. 71326, respectively, or a homolog or a fragment thereof, is increased or generated, or if the activity 30S ribosomal protein S3 is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 36 to 65-percent is conferred as compared to a corresponding non-transformed wild type non-human organism.

Accordingly, in one embodiment, an increase of asparagine in a non-human organism, as compared to a corresponding non-transformed wild type non-human organism, is conferred in the process of the invention, if the activity of a polypeptide showing the activity of a 50S ribosomal protein L14, or if the activity of the polypeptide AvinDRAFT_{—}2369, preferably represented by SEQ ID NO. 71764, or a homolog or fragment thereof, or if the activity of a polypeptide encoded by a nucleic acid molecule comprising the nucleic acid SEQ ID NO. 71763, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 71763 or polypeptide SEQ ID NO. 71764, respectively, or a homolog or a fragment thereof, is increased or generated, or if the activity 50S ribosomal protein L14 is increased or generated in a non-human organism, like a microorganism or a plant cell, plant or part thereof, especially with non-targeted localization, whereby the respective line discloses in column 7 asparagine. For example, an increase of the asparagine of at least 1 percent, particularly in a range of 62 to 91-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a ABC transporter permease protein, or if the activity of the polypeptide AvinDRAFT_{—}2521, preferably represented by SEQ ID NO. 27883, or a homolog or fragment thereof, or if the activity of a polypeptide encoded by a nucleic acid molecule comprising the nucleic acid SEQ ID NO. 27882, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 27882 or polypeptide SEQ ID NO. 27883, respectively, or a homolog or a fragment thereof, is increased or generated, or if the activity ABC transporter permease protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 59 to 193-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a acyl-CoA synthase, or if the activity of the polypeptide AvinDRAFT_{—}2754, preferably represented by SEQ ID NO. 28041, or a homolog or fragment thereof, or if the activity of a polypeptide encoded by a nucleic acid molecule comprising the nucleic acid SEQ ID NO. 28040, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 28040 or polypeptide SEQ ID NO. 28041, respectively, or a homolog or a fragment thereof, is increased or generated, or if the activity acyl-CoA synthase is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 96 to 314-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a acyl-CoA synthase, or if the activity of the polypeptide AvinDRAFT_{—}2754, preferably represented by SEQ ID NO. 28041, or a homolog or fragment thereof, or if the activity of a polypeptide encoded by a nucleic acid molecule comprising the nucleic acid SEQ ID NO. 28040, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 28040 or polypeptide SEQ ID NO. 28041, respectively, or a homolog or a fragment thereof, is increased or generated, or if the activity acyl-CoA synthase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 48 to 95-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a acyl-CoA synthase, or if the activity of the polypeptide AvinDRAFT_{—}2754, preferably represented by SEQ ID NO. 28041, or a homolog or fragment thereof, or if the activity of a polypeptide encoded by a nucleic acid molecule comprising the nucleic acid SEQ ID NO. 28040, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 28040 or polypeptide SEQ ID NO. 28041, respectively, or a homolog or a fragment thereof, is increased or generated, or if the activity acyl-CoA synthase is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 34 to 112-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a 2-oxoglutarate dehydrogenase E1 subunit, or if the activity of the polypeptide AvinDRAFT_{—}3028, preferably represented by SEQ ID NO. 6076, or a homolog or fragment thereof, or if the activity of a polypeptide encoded by a nucleic acid molecule comprising the nucleic acid SEQ ID NO. 6075, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 6075 or polypeptide SEQ ID NO. 6076, respectively, or a homolog or a fragment thereof, is increased or generated, or if the activity 2-oxoglutarate dehydrogenase E1 subunit is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 52 to 251-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a 2-oxoglutarate dehydrogenase E1 subunit, or if the activity of the polypeptide AvinDRAFT_{—}3028, preferably represented by SEQ ID NO. 6076, or a homolog or fragment thereof, or if the activity of a polypeptide encoded by a nucleic acid molecule comprising the nucleic acid SEQ ID NO. 6075, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 6075 or polypeptide SEQ ID NO. 6076, respectively, or a homolog or a fragment thereof, is increased or generated, or if the activity 2-oxoglutarate dehydrogenase E1 subunit is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 41 to 110-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a GDP-mannose dehydrogenase, or if the activity of the polypeptide AvinDRAFT_{—}3135, preferably represented by SEQ ID NO. 72347, or a homolog or fragment thereof, or if the activity of a polypeptide encoded by a nucleic acid molecule comprising the nucleic acid SEQ ID NO. 72346, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 72346 or polypeptide SEQ ID NO. 72347, respectively, or a homolog or a fragment thereof, is increased or generated, or if the activity GDP-mannose dehydrogenase is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 28 to 60-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a transcriptional regulator, or if the activity of the polypeptide AvinDRAFT_{—}3186, preferably represented by SEQ ID NO. 29247, or a homolog or fragment thereof, or if the activity of a polypeptide encoded by a nucleic acid molecule comprising the nucleic acid SEQ ID NO. 29246, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 29246 or polypeptide SEQ ID NO. 29247, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 34 to 44-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a transcriptional regulator, or if the activity of the polypeptide AvinDRAFT_{—}3209, preferably represented by SEQ ID NO. 29287, or a homolog or fragment thereof, or if the activity of a polypeptide encoded by a nucleic acid molecule comprising the nucleic acid SEQ ID NO. 29286, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 29286 or polypeptide SEQ ID NO. 29287, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 45 to 115-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a oxidoreductase, or if the activity of the polypeptide AvinDRAFT_{—}3253, preferably represented by SEQ ID NO. 29501, or a homolog or fragment thereof, or if the activity of a polypeptide encoded by a nucleic acid molecule comprising the nucleic acid SEQ ID NO. 29500, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 29500 or polypeptide SEQ ID NO. 29501, respectively, or a homolog or a fragment thereof, is increased or generated, or if the activity oxidoreductase is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 60 to 110-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a oxidoreductase, or if the activity of the polypeptide AvinDRAFT_{—}3253, preferably represented by SEQ ID NO. 29501, or a homolog or fragment thereof, or if the activity of a polypeptide encoded by a nucleic acid molecule comprising the nucleic acid SEQ ID NO. 29500, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 29500 or polypeptide SEQ ID NO. 29501, respectively, or a homolog or a fragment thereof, is increased or generated, or if the activity oxidoreductase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 39 to 99-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a lysyl-tRNA synthetase, or if the activity of the polypeptide AvinDRAFT_{—}3556, preferably represented by SEQ ID NO. 30040, or a homolog or fragment thereof, or if the activity of a polypeptide encoded by a nucleic acid molecule comprising the nucleic acid SEQ ID NO. 30039, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 30039 or polypeptide SEQ ID NO. 30040, respectively, or a homolog or a fragment thereof, is increased or generated, or if the activity lysyl-tRNA synthetase is increased or generated in a non-human organism, like a microorganism or a plant cell, plant or part thereof, especially with non-targeted localization, whereby the respective line discloses in column 7 citrulline. For example, an increase of the citrulline of at least 1 percent, particularly in a range of 30 to 54-percent is conferred as compared to a corresponding non-transformed wild type non-human organism.

Accordingly, in one embodiment, an increase of asparagine in a non-human organism, as compared to a corresponding non-transformed wild type non-human organism, is conferred in the process of the invention, if the activity of a polypeptide showing the activity of a ABC transporter component, or if the activity of the polypeptide AVINDRAFT_{—}4128, preferably represented by SEQ ID NO. 72521, or a homolog or fragment thereof, or if the activity of a polypeptide encoded by a nucleic acid molecule comprising the nucleic acid SEQ ID NO. 72520, preferably the coding region thereof, or a homolog or fragment thereof, e.g. derived from AZOTOBACTER VINELANDII, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 72520 or polypeptide SEQ ID NO. 72521, respectively, or a homolog or a fragment thereof, is increased or generated, or if the activity ABC transporter component is increased or generated in a non-human organism, like a microorganism or a plant cell, plant or part thereof, especially with non-targeted localization, whereby the respective line discloses in column 7 asparagine. For example, an increase of the asparagine of at least 1 percent, particularly in a range of 49 to 246-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a glucose-1-phosphate cytidylyltransferase, or if the activity of the polypeptide AvinDRAFT_{—}4384, preferably represented by SEQ ID NO. 31718, or a homolog or fragment thereof, or if the activity of a polypeptide encoded by a nucleic acid molecule comprising the nucleic acid SEQ ID NO. 31717, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 31717 or polypeptide SEQ ID NO. 31718, respectively, or a homolog or a fragment thereof, is increased or generated, or if the activity glucose-1-phosphate cytidylyltransferase is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 75 to 152-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a CDP-glucose-4,6-dehydratase, or if the activity of the polypeptide AvinDRAFT_{—}4385, preferably represented by SEQ ID NO. 72936, or a homolog or fragment thereof, or if the activity of a polypeptide encoded by a nucleic acid molecule comprising the nucleic acid SEQ ID NO. 72935, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 72935 or polypeptide SEQ ID NO. 72936, respectively, or a homolog or a fragment thereof, is increased or generated, or if the activity CDP-glucose-4,6-dehydratase is increased or generated in a non-human organism, like a microorganism or a plant cell, plant or part thereof, especially with non-targeted localization, whereby the respective line discloses in column 7 glycine. For example, an increase of the glycine of at least 1 percent, particularly in a range of 68 to 241-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a enoyl-CoA hydratase, or if the activity of the polypeptide AvinDRAFT_{—}4420, preferably represented by SEQ ID NO. 73039, or a homolog or fragment thereof, or if the activity of a polypeptide encoded by a nucleic acid molecule comprising the nucleic acid SEQ ID NO. 73038, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 73038 or polypeptide SEQ ID NO. 73039, respectively, or a homolog or a fragment thereof, is increased or generated, or if the activity enoyl-CoA hydratase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 27 to 111-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a glutathione peroxidase, or if the activity of the polypeptide AvinDRAFT_{—}4446, preferably represented by SEQ ID NO. 73285, or a homolog or fragment thereof, or if the activity of a polypeptide encoded by a nucleic acid molecule comprising the nucleic acid SEQ ID NO. 73284, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 73284 or polypeptide SEQ ID NO. 73285, respectively, or a homolog or a fragment thereof, is increased or generated, or if the activity glutathione peroxidase is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 19 to 35-percent is conferred as compared to a corresponding non-transformed wild type non-human organism.

Accordingly, in one embodiment, an increase of asparagine in a non-human organism, as compared to a corresponding non-transformed wild type non-human organism, is conferred in the process of the invention, if the activity of a polypeptide showing the activity of a aminotransferase, or if the activity of the polypeptide AvinDRAFT_{—}4562, preferably represented by SEQ ID NO. 31927, or a homolog or fragment thereof, or if the activity of a polypeptide encoded by a nucleic acid molecule comprising the nucleic acid SEQ ID NO. 31926, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 31926 or polypeptide SEQ ID NO. 31927, respectively, or a homolog or a fragment thereof, is increased or generated, or if the activity aminotransferase is increased or generated in a non-human organism, like a microorganism or a plant cell, plant or part thereof, especially with non-targeted localization, whereby the respective line discloses in column 7 asparagine. For example, an increase of the asparagine of at least 1 percent, particularly in a range of 41 to 243-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a aminotransferase, or if the activity of the polypeptide AvinDRAFT_{—}4562, preferably represented by SEQ ID NO. 31927, or a homolog or fragment thereof, or if the activity of a polypeptide encoded by a nucleic acid molecule comprising the nucleic acid SEQ ID NO. 31926, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 31926 or polypeptide SEQ ID NO. 31927, respectively, or a homolog or a fragment thereof, is increased or generated, or if the activity aminotransferase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 34 to 69-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a NADH-quinone oxidoreductase subunit, or if the activity of the polypeptide AvinDRAFT_{—}4606, preferably represented by SEQ ID NO. 73720, or a homolog or fragment thereof, or if the activity of a polypeptide encoded by a nucleic acid molecule comprising the nucleic acid SEQ ID NO. 73719, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 73719 or polypeptide SEQ ID NO. 73720, respectively, or a homolog or a fragment thereof, is increased or generated, or if the activity NADH-quinone oxidoreductase subunit is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 36 to 126 percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a glutamate-ammonia-ligase, or if the activity of the polypeptide AvinDRAFT_{—}4836, preferably represented by SEQ ID NO. 74164, or a homolog or fragment thereof, or if the activity of a polypeptide encoded by a nucleic acid molecule comprising the nucleic acid SEQ ID NO. 74163, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 74163 or polypeptide SEQ ID NO. 74164, respectively, or a homolog or a fragment thereof, is increased or generated, or if the activity glutamate-ammonia-ligase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 78 to 218-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a glutamate-ammonia-ligase, or if the activity of the polypeptide AvinDRAFT_{—}4836, preferably represented by SEQ ID NO. 74164, or a homolog or fragment thereof, or if the activity of a polypeptide encoded by a nucleic acid molecule comprising the nucleic acid SEQ ID NO. 74163, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 74163 or polypeptide SEQ ID NO. 74164, respectively, or a homolog or a fragment thereof, is increased or generated, or if the activity glutamate-ammonia-ligase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 42 to 162-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a RNA polymerase sigma factor, or if the activity of the polypeptide AvinDRAFT_{—}4989, preferably represented by SEQ ID NO. 74453, or a homolog or fragment thereof, or if the activity of a polypeptide encoded by a nucleic acid molecule comprising the nucleic acid SEQ ID NO. 74452, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 74452 or polypeptide SEQ ID NO. 74453, respectively, or a homolog or a fragment thereof, is increased or generated, or if the activity RNA polymerase sigma factor is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 32 to 62-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a hydrolase, or if the activity of the polypeptide AvinDRAFT_{—}5103, preferably represented by SEQ ID NO. 6511, or a homolog or fragment thereof, or if the activity of a polypeptide encoded by a nucleic acid molecule comprising the nucleic acid SEQ ID NO. 6510, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 6510 or polypeptide SEQ ID NO. 6511, respectively, or a homolog or a fragment thereof, is increased or generated, or if the activity hydrolase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 26 to 3660-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a hydrolase, or if the activity of the polypeptide AvinDRAFT_{—}5103, preferably represented by SEQ ID NO. 6511, or a homolog or fragment thereof, or if the activity of a polypeptide encoded by a nucleic acid molecule comprising the nucleic acid SEQ ID NO. 6510, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 6510 or polypeptide SEQ ID NO. 6511, respectively, or a homolog or a fragment thereof, is increased or generated, or if the activity hydrolase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 51 to 3096-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a hydrolase, or if the activity of the polypeptide AvinDRAFT_{—}5103, preferably represented by SEQ ID NO. 6511, or a homolog or fragment thereof, or if the activity of a polypeptide encoded by a nucleic acid molecule comprising the nucleic acid SEQ ID NO. 6510, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 6510 or polypeptide SEQ ID NO. 6511, respectively, or a homolog or a fragment thereof, is increased or generated, or if the activity hydrolase is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 34 to 205-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a glutamate-ammonia-ligase, or if the activity of the polypeptide AvinDRAFT_{—}5246, preferably represented by SEQ ID NO. 32038, or a homolog or fragment thereof, or if the activity of a polypeptide encoded by a nucleic acid molecule comprising the nucleic acid SEQ ID NO. 32037, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 32037 or polypeptide SEQ ID NO. 32038, respectively, or a homolog or a fragment thereof, is increased or generated, or if the activity glutamate-ammonia-ligase is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 106 to 126-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a glutamate-ammonia-ligase, or if the activity of the polypeptide AvinDRAFT_{—}5246, preferably represented by SEQ ID NO. 32038, or a homolog or fragment thereof, or if the activity of a polypeptide encoded by a nucleic acid molecule comprising the nucleic acid SEQ ID NO. 32037, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 32037 or polypeptide SEQ ID NO. 32038, respectively, or a homolog or a fragment thereof, is increased or generated, or if the activity glutamate-ammonia-ligase is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 35 to 147-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a glutamate-ammonia-ligase, or if the activity of the polypeptide AvinDRAFT_{—}5246, preferably represented by SEQ ID NO. 32038, or a homolog or fragment thereof, or if the activity of a polypeptide encoded by a nucleic acid molecule comprising the nucleic acid SEQ ID NO. 32037, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 32037 or polypeptide SEQ ID NO. 32038, respectively, or a homolog or a fragment thereof, is increased or generated, or if the activity glutamate-ammonia-ligase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 41 to 131-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a fumarylacetoacetate hydrolase, or if the activity of the polypeptide AvinDRAFT_{—}5292, preferably represented by SEQ ID NO. 32309, or a homolog or fragment thereof, or if the activity of a polypeptide encoded by a nucleic acid molecule comprising the nucleic acid SEQ ID NO. 32308, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 32308 or polypeptide SEQ ID NO. 32309, respectively, or a homolog or a fragment thereof, is increased or generated, or if the activity fumarylacetoacetate hydrolase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 36 to 246-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a fumarylacetoacetate hydrolase, or if the activity of the polypeptide AvinDRAFT_{—}5292, preferably represented by SEQ ID NO. 32309, or a homolog or fragment thereof, or if the activity of a polypeptide encoded by a nucleic acid molecule comprising the nucleic acid SEQ ID NO. 32308, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 32308 or polypeptide SEQ ID NO. 32309, respectively, or a homolog or a fragment thereof, is increased or generated, or if the activity fumarylacetoacetate hydrolase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 35 to 324-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a HesB/YadR/YfhF family protein, or if the activity of the polypeptide AvinDRAFT_{—}5467, preferably represented by SEQ ID NO. 32649, or a homolog or fragment thereof, or if the activity of a polypeptide encoded by a nucleic acid molecule comprising the nucleic acid SEQ ID NO. 32648, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 32648 or polypeptide SEQ ID NO. 32649, respectively, or a homolog or a fragment thereof, is increased or generated, or if the activity HesB/YadR/YfhF family protein is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 47 to 78-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a peptidase, or if the activity of the polypeptide AvinDRAFT_{—}5579, preferably represented by SEQ ID NO. 74663, or a homolog or fragment thereof, or if the activity of a polypeptide encoded by a nucleic acid molecule comprising the nucleic acid SEQ ID NO. 74662, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 74662 or polypeptide SEQ ID NO. 74663, respectively, or a homolog or a fragment thereof, is increased or generated, or if the activity peptidase is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 56 to 110-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a L-aspartate oxidase, or if the activity of the polypeptide AvinDRAFT_{—}5644, preferably represented by SEQ ID NO. 33086, or a homolog or fragment thereof, or if the activity of a polypeptide encoded by a nucleic acid molecule comprising the nucleic acid SEQ ID NO. 33085, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 33085 or polypeptide SEQ ID NO. 33086, respectively, or a homolog or a fragment thereof, is increased or generated, or if the activity L-aspartate oxidase is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 52 to 122-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a Chaperone protein CIpB, or if the activity of the polypeptide AvinDRAFT_{—}6093, preferably represented by SEQ ID NO. 33597, or a homolog or fragment thereof, or if the activity of a polypeptide encoded by a nucleic acid molecule comprising the nucleic acid SEQ ID NO. 33596, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 33596 or polypeptide SEQ ID NO. 33597, respectively, or a homolog or a fragment thereof, is increased or generated, or if the activity Chaperone protein CIpB is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 39 to 90-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a electron transfer flavoprotein subunit beta, or if the activity of the polypeptide AvinDRAFT_{—}6679, preferably represented by SEQ ID NO. 74730, or a homolog or fragment thereof, or if the activity of a polypeptide encoded by a nucleic acid molecule comprising the nucleic acid SEQ ID NO. 74729, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 74729 or polypeptide SEQ ID NO. 74730, respectively, or a homolog or a fragment thereof, is increased or generated, or if the activity electron transfer flavoprotein subunit beta is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 46 to 143-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a electron transfer flavoprotein subunit beta, or if the activity of the polypeptide AvinDRAFT_{—}6679, preferably represented by SEQ ID NO. 74730, or a homolog or fragment thereof, or if the activity of a polypeptide encoded by a nucleic acid molecule comprising the nucleic acid SEQ ID NO. 74729, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 74729 or polypeptide SEQ ID NO. 74730, respectively, or a homolog or a fragment thereof, is increased or generated, or if the activity electron transfer flavoprotein subunit beta is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 50 to 143-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a AX653549-protein, or if the activity of the polypeptide AX653549, preferably represented by SEQ ID NO. 34302, or a homolog or fragment thereof, or if the activity of a polypeptide encoded by a nucleic acid molecule comprising the nucleic acid SEQ ID NO. 34301, preferably the coding region thereof, or a homolog or fragment thereof, e.g. derived from Oryza sativa, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 34301 or polypeptide SEQ ID NO. 34302, respectively, or a homolog or a fragment thereof, is increased or generated, or if the activity AX653549-protein is increased or generated in a non-human organism, like a microorganism or a plant cell, plant or part thereof, especially with non-targeted localization, whereby the respective line discloses in column 7 citrulline. For example, an increase of the citrulline of at least 1 percent, particularly in a range of 39 to 48-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a AX653549-protein, or if the activity of the polypeptide AX653549, preferably represented by SEQ ID NO. 34302, or a homolog or fragment thereof, or if the activity of a polypeptide encoded by a nucleic acid molecule comprising the nucleic acid SEQ ID NO. 34301, preferably the coding region thereof, or a homolog or fragment thereof, e.g. derived from Oryza sativa, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 34301 or polypeptide SEQ ID NO. 34302, respectively, or a homolog or a fragment thereof, is increased or generated, or if the activity AX653549-protein is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 43 to 93-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a AY087308-protein, or if the activity of the polypeptide AY087308, preferably represented by SEQ ID NO. 34603, or a homolog or fragment thereof, or if the activity of a polypeptide encoded by a nucleic acid molecule comprising the nucleic acid SEQ ID NO. 34602, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 34602 or polypeptide SEQ ID NO. 34603, respectively, or a homolog or a fragment thereof, is increased or generated, or if the activity AY087308-protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 57 to 119-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a AY087308-protein, or if the activity of the polypeptide AY087308, preferably represented by SEQ ID NO. 34603, or a homolog or fragment thereof, or if the activity of a polypeptide encoded by a nucleic acid molecule comprising the nucleic acid SEQ ID NO. 34602, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 34602 or polypeptide SEQ ID NO. 34603, respectively, or a homolog or a fragment thereof, is increased or generated, or if the activity AY087308-protein is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 36 to 71-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a threonine synthase, or if the activity of the polypeptide B0004, preferably represented by SEQ ID NO. 34890, or a homolog or fragment thereof, or if the activity of a polypeptide encoded by a nucleic acid molecule comprising the nucleic acid SEQ ID NO. 34889, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 34889 or polypeptide SEQ ID NO. 34890, respectively, or a homolog or a fragment thereof, is increased or generated, or if the activity threonine synthase is increased or generated in a non-human organism, like a microorganism or a plant cell, plant or part thereof, especially with plastidic localization, whereby the respective line discloses in column 7 alanine. For example, an increase of the alanine of at least 1 percent, particularly in a range of 29 to 147-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a threonine synthase, or if the activity of the polypeptide B0004, preferably represented by SEQ ID NO. 34890, or a homolog or fragment thereof, or if the activity of a polypeptide encoded by a nucleic acid molecule comprising the nucleic acid SEQ ID NO. 34889, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 34889 or polypeptide SEQ ID NO. 34890, respectively, or a homolog or a fragment thereof, is increased or generated, or if the activity threonine synthase is increased or generated in a non-human organism, like a microorganism or a plant cell, plant or part thereof, especially with plastidic localization, whereby the respective line discloses in column 7 phenylalanine. For example, an increase of the phenylalanine of at least 1 percent, particularly in a range of 30 to 200-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a threonine synthase, or if the activity of the polypeptide B0004, preferably represented by SEQ ID NO. 34890, or a homolog or fragment thereof, or if the activity of a polypeptide encoded by a nucleic acid molecule comprising the nucleic acid SEQ ID NO. 34889, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 34889 or polypeptide SEQ ID NO. 34890, respectively, or a homolog or a fragment thereof, is increased or generated, or if the activity threonine synthase is increased or generated in a non-human organism, like a microorganism or a plant cell, plant or part thereof, especially with plastidic localization, whereby the respective line discloses in column 7 serine. For example, an increase of the serine of at least 1 percent, particularly in a range of 23 to 195-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a threonine synthase, or if the activity of the polypeptide B0004, preferably represented by SEQ ID NO. 34890, or a homolog or fragment thereof, or if the activity of a polypeptide encoded by a nucleic acid molecule comprising the nucleic acid SEQ ID NO. 34889, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 34889 or polypeptide SEQ ID NO. 34890, respectively, or a homolog or a fragment thereof, is increased or generated, or if the activity threonine synthase is increased or generated in a non-human organism, like a microorganism or a plant cell, plant or part thereof, especially with plastidic localization, whereby the respective line discloses in column 7 tyrosine. For example, an increase of the tyrosine of at least 1 percent, particularly in a range of 37 to 458-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a threonine synthase, or if the activity of the polypeptide B0004, preferably represented by SEQ ID NO. 34890, or a homolog or fragment thereof, or if the activity of a polypeptide encoded by a nucleic acid molecule comprising the nucleic acid SEQ ID NO. 34889, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 34889 or polypeptide SEQ ID NO. 34890, respectively, or a homolog or a fragment thereof, is increased or generated, or if the activity threonine synthase is increased or generated in a non-human organism, like a microorganism or a plant cell, plant or part thereof, especially with plastidic localization, whereby the respective line discloses in column 7 5-oxoproline. For example, an increase of the 5-oxoproline of at least 1 percent, particularly in a range of 44 to 1268-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a acetolactate synthase small subunit, or if the activity of the polypeptide B0078, preferably represented by SEQ ID NO. 6819, or a homolog or fragment thereof, or if the activity of a polypeptide encoded by a nucleic acid molecule comprising the nucleic acid SEQ ID NO. 6818, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 6818 or polypeptide SEQ ID NO. 6819, respectively, or a homolog or a fragment thereof, is increased or generated, or if the activity acetolactate synthase small subunit is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 32 to 59-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a glucose dehydrogenase, or if the activity of the polypeptide B0124, preferably represented by SEQ ID NO. 35483, or a homolog or fragment thereof, or if the activity of a polypeptide encoded by a nucleic acid molecule comprising the nucleic acid SEQ ID NO. 35482, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 35482 or polypeptide SEQ ID NO. 35483, respectively, or a homolog or a fragment thereof, is increased or generated, or if the activity glucose dehydrogenase is increased or generated in a non-human organism, like a microorganism or a plant cell, plant or part thereof, especially with non-targeted localization, whereby the respective line discloses in column 7 glycine. For example, an increase of the glycine of at least 1 percent, particularly in a range of 31 to 179-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a glucose dehydrogenase, or if the activity of the polypeptide B0124, preferably represented by SEQ ID NO. 35483, or a homolog or fragment thereof, or if the activity of a polypeptide encoded by a nucleic acid molecule comprising the nucleic acid SEQ ID NO. 35482, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 35482 or polypeptide SEQ ID NO. 35483, respectively, or a homolog or a fragment thereof, is increased or generated, or if the activity glucose dehydrogenase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 36 to 557-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a glucose dehydrogenase, or if the activity of the polypeptide B0124, preferably represented by SEQ ID NO. 35483, or a homolog or fragment thereof, or if the activity of a polypeptide encoded by a nucleic acid molecule comprising the nucleic acid SEQ ID NO. 35482, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 35482 or polypeptide SEQ ID NO. 35483, respectively, or a homolog or a fragment thereof, is increased or generated, or if the activity glucose dehydrogenase is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 55 to 171-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a glucose dehydrogenase, or if the activity of the polypeptide B0124, preferably represented by SEQ ID NO. 35483, or a homolog or fragment thereof, or if the activity of a polypeptide encoded by a nucleic acid molecule comprising the nucleic acid SEQ ID NO. 35482, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 35482 or polypeptide SEQ ID NO. 35483, respectively, or a homolog or a fragment thereof, is increased or generated, or if the activity glucose dehydrogenase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 36 to 265-percent is conferred as compared to a corresponding non-transformed wild type non-human organism.

Accordingly, in one embodiment, an increase of asparagine in a non-human organism, as compared to a corresponding non-transformed wild type non-human organism, is conferred in the process of the invention, if the activity of a polypeptide showing the activity of a aspartate 1-decarboxylase, or if the activity of the polypeptide B0131, preferably represented by SEQ ID NO. 74987, or a homolog or fragment thereof, or if the activity of a polypeptide encoded by a nucleic acid molecule comprising the nucleic acid SEQ ID NO. 74986, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 74986 or polypeptide SEQ ID NO. 74987, respectively, or a homolog or a fragment thereof, is increased or generated, or if the activity aspartate 1-decarboxylase is increased or generated in a non-human organism, like a microorganism or a plant cell, plant or part thereof, especially with non-targeted localization, whereby the respective line discloses in column 7 asparagine. For example, an increase of the asparagine of at least 1 percent, particularly in a range of 49 to 85-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a aspartate 1-decarboxylase, or if the activity of the polypeptide B0131, preferably represented by SEQ ID NO. 74987, or a homolog or fragment thereof, or if the activity of a polypeptide encoded by a nucleic acid molecule comprising the nucleic acid SEQ ID NO. 74986, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 74986 or polypeptide SEQ ID NO. 74987, respectively, or a homolog or a fragment thereof, is increased or generated, or if the activity aspartate 1-decarboxylase is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 23 to 55-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a serine protease, or if the activity of the polypeptide B0161, preferably represented by SEQ ID NO. 7082, or a homolog or fragment thereof, or if the activity of a polypeptide encoded by a nucleic acid molecule comprising the nucleic acid SEQ ID NO. 7081, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7081 or polypeptide SEQ ID NO. 7082, respectively, or a homolog or a fragment thereof, is increased or generated, or if the activity serine protease is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 19 to 58-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a serine protease, or if the activity of the polypeptide B0161, preferably represented by SEQ ID NO. 7082, or a homolog or fragment thereof, or if the activity of a polypeptide encoded by a nucleic acid molecule comprising the nucleic acid SEQ ID NO. 7081, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7081 or polypeptide SEQ ID NO. 7082, respectively, or a homolog or a fragment thereof, is increased or generated, or if the activity serine protease is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 42 to 103-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a serine protease, or if the activity of the polypeptide B0161, preferably represented by SEQ ID NO. 7082, or a homolog or fragment thereof, or if the activity of a polypeptide encoded by a nucleic acid molecule comprising the nucleic acid SEQ ID NO. 7081, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7081 or polypeptide SEQ ID NO. 7082, respectively, or a homolog or a fragment thereof, is increased or generated, or if the activity serine protease is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 277 to 1628-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a acyl-CoA dehydrogenase, or if the activity of the polypeptide B0221, preferably represented by SEQ ID NO. 35591, or a homolog or fragment thereof, or if the activity of a polypeptide encoded by a nucleic acid molecule comprising the nucleic acid SEQ ID NO. 35590, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 35590 or polypeptide SEQ ID NO. 35591, respectively, or a homolog or a fragment thereof, is increased or generated, or if the activity acyl-CoA dehydrogenase is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 25 to 63-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a acyl-CoA dehydrogenase, or if the activity of the polypeptide B0221, preferably represented by SEQ ID NO. 35591, or a homolog or fragment thereof, or if the activity of a polypeptide encoded by a nucleic acid molecule comprising the nucleic acid SEQ ID NO. 35590, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 35590 or polypeptide SEQ ID NO. 35591, respectively, or a homolog or a fragment thereof, is increased or generated, or if the activity acyl-CoA dehydrogenase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 39 to 107-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a beta-galactosidase, or if the activity of the polypeptide B0344, preferably represented by SEQ ID NO. 35734, or a homolog or fragment thereof, or if the activity of a polypeptide encoded by a nucleic acid molecule comprising the nucleic acid SEQ ID NO. 35733, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 35733 or polypeptide SEQ ID NO. 35734, respectively, or a homolog or a fragment thereof, is increased or generated, or if the activity beta-galactosidase is increased or generated in a non-human organism, like a microorganism or a plant cell, plant or part thereof, especially with plastidic localization, whereby the respective line discloses in column 7 tyrosine. For example, an increase of the tyrosine of at least 1 percent, particularly in a range of 34 to 103-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a beta-galactosidase, or if the activity of the polypeptide B0344, preferably represented by SEQ ID NO. 35734, or a homolog or fragment thereof, or if the activity of a polypeptide encoded by a nucleic acid molecule comprising the nucleic acid SEQ ID NO. 35733, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 35733 or polypeptide SEQ ID NO. 35734, respectively, or a homolog or a fragment thereof, is increased or generated, or if the activity beta-galactosidase is increased or generated in a non-human organism, like a microorganism or a plant cell, plant or part thereof, especially with plastidic localization, whereby the respective line discloses in column 7 5-oxoproline. For example, an increase of the 5-oxoproline of at least 1 percent, particularly in a range of 31 to 40-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a 2,3-dihydroxyphenylpropionate 1,2-dioxygenase, or if the activity of the polypeptide B0348, preferably represented by SEQ ID NO. 7270, or a homolog or fragment thereof, or if the activity of a polypeptide encoded by a nucleic acid molecule comprising the nucleic acid SEQ ID NO. 7269, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7269 or polypeptide SEQ ID NO. 7270, respectively, or a homolog or a fragment thereof, is increased or generated, or if the activity 2,3-dihydroxyphenylpropionate 1,2-dioxygenase is increased or generated in a non-human organism, like a microorganism or a plant cell, plant or part thereof, especially with plastidic localization, whereby the respective line discloses in column 7 glycine. For example, an increase of the glycine of at least 1 percent, particularly in a range of 96 to 301-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a 2,3-dihydroxyphenylpropionate 1,2-dioxygenase, or if the activity of the polypeptide B0348, preferably represented by SEQ ID NO. 7270, or a homolog or fragment thereof, or if the activity of a polypeptide encoded by a nucleic acid molecule comprising the nucleic acid SEQ ID NO. 7269, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7269 or polypeptide SEQ ID NO. 7270, respectively, or a homolog or a fragment thereof, is increased or generated, or if the activity 2,3-dihydroxyphenylpropionate 1,2-dioxygenase is increased or generated in a non-human organism, like a microorganism or a plant cell, plant or part thereof, especially with plastidic localization, whereby the respective line discloses in column 7 phenylalanine. For example, an increase of the phenylalanine of at least 1 percent, particularly in a range of 72 to 197-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a 2,3-dihydroxyphenylpropionate 1,2-dioxygenase, or if the activity of the polypeptide B0348, preferably represented by SEQ ID NO. 7270, or a homolog or fragment thereof, or if the activity of a polypeptide encoded by a nucleic acid molecule comprising the nucleic acid SEQ ID NO. 7269, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7269 or polypeptide SEQ ID NO. 7270, respectively, or a homolog or a fragment thereof, is increased or generated, or if the activity 2,3-dihydroxyphenylpropionate 1,2-dioxygenase is increased or generated in a non-human organism, like a microorganism or a plant cell, plant or part thereof, especially with plastidic localization, whereby the respective line discloses in column 7 serine. For example, an increase of the serine of at least 1 percent, particularly in a range of 38 to 320-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a 2,3-dihydroxyphenylpropionate 1,2-dioxygenase, or if the activity of the polypeptide B0348, preferably represented by SEQ ID NO. 7270, or a homolog or fragment thereof, or if the activity of a polypeptide encoded by a nucleic acid molecule comprising the nucleic acid SEQ ID NO. 7269, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7269 or polypeptide SEQ ID NO. 7270, respectively, or a homolog or a fragment thereof, is increased or generated, or if the activity 2,3-dihydroxyphenylpropionate 1,2-dioxygenase is increased or generated in a non-human organism, like a microorganism or a plant cell, plant or part thereof, especially with plastidic localization, whereby the respective line discloses in column 7 tyrosine. For example, an increase of the tyrosine of at least 1 percent, particularly in a range of 67 to 83-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a b0391-protein, or if the activity of the polypeptide B0391, preferably represented by SEQ ID NO. 75196, or a homolog or fragment thereof, or if the activity of a polypeptide encoded by a nucleic acid molecule comprising the nucleic acid SEQ ID NO. 75195, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 75195 or polypeptide SEQ ID NO. 75196, respectively, or a homolog or a fragment thereof, is increased or generated, or if the activity b0391-protein is increased or generated in a non-human organism, like a microorganism or a plant cell, plant or part thereof, especially with plastidic localization, whereby the respective line discloses in column 7 aspartate. For example, an increase of the aspartate of at least 1 percent, particularly in a range of 56 to 93-percent is conferred as compared to a corresponding non-transformed wild type non-human organism.

Accordingly, in one embodiment, an increase of asparagine in a non-human organism, as compared to a corresponding non-transformed wild type non-human organism, is conferred in the process of the invention, if the activity of a polypeptide showing the activity of a ATP-binding component of a transport system, or if the activity of the polypeptide B0449, preferably represented by SEQ ID NO. 7334, or a homolog or fragment thereof, or if the activity of a polypeptide encoded by a nucleic acid molecule comprising the nucleic acid SEQ ID NO. 7333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7333 or polypeptide SEQ ID NO. 7334, respectively, or a homolog or a fragment thereof, is increased or generated, or if the activity ATP-binding component of a transport system is increased or generated in a non-human organism, like a microorganism or a plant cell, plant or part thereof, especially with non-targeted localization, whereby the respective line discloses in column 7 asparagine. For example, an increase of the asparagine of at least 1 percent, particularly in a range of 51 to 684-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a ATP-binding component of a transport system, or if the activity of the polypeptide B0449, preferably represented by SEQ ID NO. 7334, or a homolog or fragment thereof, or if the activity of a polypeptide encoded by a nucleic acid molecule comprising the nucleic acid SEQ ID NO. 7333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7333 or polypeptide SEQ ID NO. 7334, respectively, or a homolog or a fragment thereof, is increased or generated, or if the activity ATP-binding component of a transport system is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 88 to 201-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a ATP-binding component of a transport system, or if the activity of the polypeptide B0449, preferably represented by SEQ ID NO. 7334, or a homolog or fragment thereof, or if the activity of a polypeptide encoded by a nucleic acid molecule comprising the nucleic acid SEQ ID NO. 7333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7333 or polypeptide SEQ ID NO. 7334, respectively, or a homolog or a fragment thereof, is increased or generated, or if the activity ATP-binding component of a transport system is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 40 to 1462-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a ATP-binding component of a transport system, or if the activity of the polypeptide B0449, preferably represented by SEQ ID NO. 7334, or a homolog or fragment thereof, or if the activity of a polypeptide encoded by a nucleic acid molecule comprising the nucleic acid SEQ ID NO. 7333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7333 or polypeptide SEQ ID NO. 7334, respectively, or a homolog or a fragment thereof, is increased or generated, or if the activity ATP-binding component of a transport system is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 29 to 113-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a ATP-binding component of a transport system, or if the activity of the polypeptide B0449, preferably represented by SEQ ID NO. 7334, or a homolog or fragment thereof, or if the activity of a polypeptide encoded by a nucleic acid molecule comprising the nucleic acid SEQ ID NO. 7333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7333 or polypeptide SEQ ID NO. 7334, respectively, or a homolog or a fragment thereof, is increased or generated, or if the activity ATP-binding component of a transport system is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 46 to 1202-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a b0456-protein, or if the activity of the polypeptide B0456, preferably represented by SEQ ID NO. 35876, or a homolog or fragment thereof, or if the activity of a polypeptide encoded by a nucleic acid molecule comprising the nucleic acid SEQ ID NO. 35875, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 35875 or polypeptide SEQ ID NO. 35876, respectively, or a homolog or a fragment thereof, is increased or generated, or if the activity b0456-protein is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 20 to 53-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a membrane transport protein, or if the activity of the polypeptide B0486, preferably represented by SEQ ID NO. 7687, or a homolog or fragment thereof, or if the activity of a polypeptide encoded by a nucleic acid molecule comprising the nucleic acid SEQ ID NO. 7686, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7686 or polypeptide SEQ ID NO. 7687, respectively, or a homolog or a fragment thereof, is increased or generated, or if the activity membrane transport protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 75 to 129-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a membrane transport protein, or if the activity of the polypeptide B0486, preferably represented by SEQ ID NO. 7687, or a homolog or fragment thereof, or if the activity of a polypeptide encoded by a nucleic acid molecule comprising the nucleic acid SEQ ID NO. 7686, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7686 or polypeptide SEQ ID NO. 7687, respectively, or a homolog or a fragment thereof, is increased or generated, or if the activity membrane transport protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 35 to 156-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a membrane transport protein, or if the activity of the polypeptide B0486, preferably represented by SEQ ID NO. 7687, or a homolog or fragment thereof, or if the activity of a polypeptide encoded by a nucleic acid molecule comprising the nucleic acid SEQ ID NO. 7686, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7686 or polypeptide SEQ ID NO. 7687, respectively, or a homolog or a fragment thereof, is increased or generated, or if the activity membrane transport protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 57 to 227-percent is conferred as compared to a corresponding non-transformed wild type non-human organism.

Accordingly, in one embodiment, an increase of homoserine in a non-human organism, as compared to a corresponding non-transformed wild type non-human organism, is conferred in the process of the invention, if the activity of a polypeptide showing the activity of a b0518-protein, or if the activity of the polypeptide B0518, preferably represented by SEQ ID NO. 35937, or a homolog or fragment thereof, or if the activity of a polypeptide encoded by a nucleic acid molecule comprising the nucleic acid SEQ ID NO. 35936, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 35936 or polypeptide SEQ ID NO. 35937, respectively, or a homolog or a fragment thereof, is increased or generated, or if the activity b0518-protein is increased or generated in a non-human organism, like a microorganism or a plant cell, plant or part thereof, especially with non-targeted localization, whereby the respective line discloses in column 7 homoserine. For example, an increase of the homoserine of at least 1 percent, particularly in a range of 24 to 72-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a isochorismate synthase, or if the activity of the polypeptide B0593, preferably represented by SEQ ID NO. 35968, or a homolog or fragment thereof, or if the activity of a polypeptide encoded by a nucleic acid molecule comprising the nucleic acid SEQ ID NO. 35967, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 35967 or polypeptide SEQ ID NO. 35968, respectively, or a homolog or a fragment thereof, is increased or generated, or if the activity isochorismate synthase is increased or generated in a non-human organism, like a microorganism or a plant cell, plant or part thereof, especially with plastidic localization, whereby the respective line discloses in column 7 aspartate. For example, an increase of the aspartate of at least 1 percent, particularly in a range of 84 to 319-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a isochorismate synthase, or if the activity of the polypeptide B0593, preferably represented by SEQ ID NO. 35968, or a homolog or fragment thereof, or if the activity of a polypeptide encoded by a nucleic acid molecule comprising the nucleic acid SEQ ID NO. 35967, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 35967 or polypeptide SEQ ID NO. 35968, respectively, or a homolog or a fragment thereof, is increased or generated, or if the activity isochorismate synthase is increased or generated in a non-human organism, like a microorganism or a plant cell, plant or part thereof, especially with plastidic localization, whereby the respective line discloses in column 7 phenylalanine. For example, an increase of the phenylalanine of at least 1 percent, particularly in a range of 37 to 256-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a isochorismate synthase, or if the activity of the polypeptide B0593, preferably represented by SEQ ID NO. 35968, or a homolog or fragment thereof, or if the activity of a polypeptide encoded by a nucleic acid molecule comprising the nucleic acid SEQ ID NO. 35967, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 35967 or polypeptide SEQ ID NO. 35968, respectively, or a homolog or a fragment thereof, is increased or generated, or if the activity isochorismate synthase is increased or generated in a non-human organism, like a microorganism or a plant cell, plant or part thereof, especially with plastidic localization, whereby the respective line discloses in column 7 serine. For example, an increase of the serine of at least 1 percent, particularly in a range of 153 to 359-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a isochorismate synthase, or if the activity of the polypeptide B0593, preferably represented by SEQ ID NO. 35968, or a homolog or fragment thereof, or if the activity of a polypeptide encoded by a nucleic acid molecule comprising the nucleic acid SEQ ID NO. 35967, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 35967 or polypeptide SEQ ID NO. 35968, respectively, or a homolog or a fragment thereof, is increased or generated, or if the activity isochorismate synthase is increased or generated in a non-human organism, like a microorganism or a plant cell, plant or part thereof, especially with plastidic localization, whereby the respective line discloses in column 7 tyrosine. For example, an increase of the tyrosine of at least 1 percent, particularly in a range of 49 to 202-percent is conferred as compared to a corresponding non-transformed wild type non-human organism.

Accordingly, in one embodiment, an increase of 3,4-dihydroxyphenylalanine (dopa) in a non-human organism, as compared to a corresponding non-transformed wild type non-human organism, is conferred in the process of the invention, if the activity of a polypeptide showing the activity of a isochorismate synthase, or if the activity of the polypeptide B0593, preferably represented by SEQ ID NO. 35968, or a homolog or fragment thereof, or if the activity of a polypeptide encoded by a nucleic acid molecule comprising the nucleic acid SEQ ID NO. 35967, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 35967 or polypeptide SEQ ID NO. 35968, respectively, or a homolog or a fragment thereof, is increased or generated, or if the activity isochorismate synthase is increased or generated in a non-human organism, like a microorganism or a plant cell, plant or part thereof, especially with plastidic localization, whereby the respective line discloses in column 7 3,4-dihydroxyphenylalanine (dopa). For example, an increase of the 3,4-dihydroxyphenylalanine (dopa) of at least 1 percent, particularly in a range of 625 to 4899-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a isochorismate synthase, or if the activity of the polypeptide B0593, preferably represented by SEQ ID NO. 35968, or a homolog or fragment thereof, or if the activity of a polypeptide encoded by a nucleic acid molecule comprising the nucleic acid SEQ ID NO. 35967, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 35967 or polypeptide SEQ ID NO. 35968, respectively, or a homolog or a fragment thereof, is increased or generated, or if the activity isochorismate synthase is increased or generated in a non-human organism, like a microorganism or a plant cell, plant or part thereof, especially with plastidic localization, whereby the respective line discloses in column 7 5-oxoproline. For example, an increase of the 5-oxoproline of at least 1 percent, particularly in a range of 100 to 636-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a zinc transporter, or if the activity of the polypeptide B0752, preferably represented by SEQ ID NO. 36115, or a homolog or fragment thereof, or if the activity of a polypeptide encoded by a nucleic acid molecule comprising the nucleic acid SEQ ID NO. 36114, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 36114 or polypeptide SEQ ID NO. 36115, respectively, or a homolog or a fragment thereof, is increased or generated, or if the activity zinc transporter is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 38 to 112-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a 3-deoxy-D-arabinoheptulosonate-7-phosphatesynthase, or if the activity of the polypeptide B0754, preferably represented by SEQ ID NO. 75287, or a homolog or fragment thereof, or if the activity of a polypeptide encoded by a nucleic acid molecule comprising the nucleic acid SEQ ID NO. 75286, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 75286 or polypeptide SEQ ID NO. 75287, respectively, or a homolog or a fragment thereof, is increased or generated, or if the activity 3-deoxy-D-arabinoheptulosonate-7-phosphatesynthase is increased or generated in a non-human organism, like a microorganism or a plant cell, plant or part thereof, especially with plastidic localization, whereby the respective line discloses in column 7 tyrosine. For example, an increase of the tyrosine of at least 1 percent, particularly in a range of 55 to 197-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a b0801-protein, or if the activity of the polypeptide B0801, preferably represented by SEQ ID NO. 75808, or a homolog or fragment thereof, or if the activity of a polypeptide encoded by a nucleic acid molecule comprising the nucleic acid SEQ ID NO. 75807, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 75807 or polypeptide SEQ ID NO. 75808, respectively, or a homolog or a fragment thereof, is increased or generated, or if the activity b0801-protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 38 to 67-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a asparaginase, or if the activity of the polypeptide B0828, preferably represented by SEQ ID NO. 36300, or a homolog or fragment thereof, or if the activity of a polypeptide encoded by a nucleic acid molecule comprising the nucleic acid SEQ ID NO. 36299, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 36299 or polypeptide SEQ ID NO. 36300, respectively, or a homolog or a fragment thereof, is increased or generated, or if the activity asparaginase is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 24 to 37-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a b0841-protein, or if the activity of the polypeptide B0841, preferably represented by SEQ ID NO. 75874, or a homolog or fragment thereof, or if the activity of a polypeptide encoded by a nucleic acid molecule comprising the nucleic acid SEQ ID NO. 75873, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 75873 or polypeptide SEQ ID NO. 75874, respectively, or a homolog or a fragment thereof, is increased or generated, or if the activity b0841-protein is increased or generated in a non-human organism, like a microorganism or a plant cell, plant or part thereof, especially with non-targeted localization, whereby the respective line discloses in column 7 glycine. For example, an increase of the glycine of at least 1 percent, particularly in a range of 40 to 72-percent is conferred as compared to a corresponding non-transformed wild type non-human organism.

Accordingly, in one embodiment, an increase of homoserine in a non-human organism, as compared to a corresponding non-transformed wild type non-human organism, is conferred in the process of the invention, if the activity of a polypeptide showing the activity of a b0841-protein, or if the activity of the polypeptide B0841, preferably represented by SEQ ID NO. 75874, or a homolog or fragment thereof, or if the activity of a polypeptide encoded by a nucleic acid molecule comprising the nucleic acid SEQ ID NO. 75873, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 75873 or polypeptide SEQ ID NO. 75874, respectively, or a homolog or a fragment thereof, is increased or generated, or if the activity b0841-protein is increased or generated in a non-human organism, like a microorganism or a plant cell, plant or part thereof, especially with non-targeted localization, whereby the respective line discloses in column 7 homoserine. For example, an increase of the homoserine of at least 1 percent, particularly in a range of 25 to 59-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a major facilitator superfamily transporter protein, or if the activity of the polypeptide B0898, preferably represented by SEQ ID NO. 7918, or a homolog or fragment thereof, or if the activity of a polypeptide encoded by a nucleic acid molecule comprising the nucleic acid SEQ ID NO. 7917, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7917 or polypeptide SEQ ID NO. 7918, respectively, or a homolog or a fragment thereof, is increased or generated, or if the activity major facilitator superfamily transporter protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 29 to 2908-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a major facilitator superfamily transporter protein, or if the activity of the polypeptide B0898, preferably represented by SEQ ID NO. 7918, or a homolog or fragment thereof, or if the activity of a polypeptide encoded by a nucleic acid molecule comprising the nucleic acid SEQ ID NO. 7917, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7917 or polypeptide SEQ ID NO. 7918, respectively, or a homolog or a fragment thereof, is increased or generated, or if the activity major facilitator superfamily transporter protein is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 34 to 290-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a major facilitator superfamily transporter protein, or if the activity of the polypeptide B0898, preferably represented by SEQ ID NO. 7918, or a homolog or fragment thereof, or if the activity of a polypeptide encoded by a nucleic acid molecule comprising the nucleic acid SEQ ID NO. 7917, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7917 or polypeptide SEQ ID NO. 7918, respectively, or a homolog or a fragment thereof, is increased or generated, or if the activity major facilitator superfamily transporter protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 41 to 2243-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a major facilitator superfamily transporter protein, or if the activity of the polypeptide B0898, preferably represented by SEQ ID NO. 7918, or a homolog or fragment thereof, or if the activity of a polypeptide encoded by a nucleic acid molecule comprising the nucleic acid SEQ ID NO. 7917, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7917 or polypeptide SEQ ID NO. 7918, respectively, or a homolog or a fragment thereof, is increased or generated, or if the activity major facilitator superfamily transporter protein is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 99 to 411-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a b0917-protein, or if the activity of the polypeptide B0917, preferably represented by SEQ ID NO. 75916, or a homolog or fragment thereof, or if the activity of a polypeptide encoded by a nucleic acid molecule comprising the nucleic acid SEQ ID NO. 75915, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 75915 or polypeptide SEQ ID NO. 75916, respectively, or a homolog or a fragment thereof, is increased or generated, or if the activity b0917-protein is increased or generated in a non-human organism, like a microorganism or a plant cell, plant or part thereof, especially with non-targeted localization, whereby the respective line discloses in column 7 citrulline. For example, an increase of the citrulline of at least 1 percent, particularly in a range of 32 to 58-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a b0917-protein, or if the activity of the polypeptide B0917, preferably represented by SEQ ID NO. 75916, or a homolog or fragment thereof, or if the activity of a polypeptide encoded by a nucleic acid molecule comprising the nucleic acid SEQ ID NO. 75915, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 75915 or polypeptide SEQ ID NO. 75916, respectively, or a homolog or a fragment thereof, is increased or generated, or if the activity b0917-protein is increased or generated in a non-human organism, like a microorganism or a plant cell, plant or part thereof, especially with non-targeted localization, whereby the respective line discloses in column 7 glycine. For example, an increase of the glycine of at least 1 percent, particularly in a range of 60 to 65-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a b0917-protein, or if the activity of the polypeptide B0917, preferably represented by SEQ ID NO. 75916, or a homolog or fragment thereof, or if the activity of a polypeptide encoded by a nucleic acid molecule comprising the nucleic acid SEQ ID NO. 75915, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 75915 or polypeptide SEQ ID NO. 75916, respectively, or a homolog or a fragment thereof, is increased or generated, or if the activity b0917-protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 32 to 68-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a b0917-protein, or if the activity of the polypeptide B0917, preferably represented by SEQ ID NO. 75916, or a homolog or fragment thereof, or if the activity of a polypeptide encoded by a nucleic acid molecule comprising the nucleic acid SEQ ID NO. 75915, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 75915 or polypeptide SEQ ID NO. 75916, respectively, or a homolog or a fragment thereof, is increased or generated, or if the activity b0917-protein is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 41 to 96-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a methylglyoxal synthase, or if the activity of the polypeptide B0963, preferably represented by SEQ ID NO. 36671, or a homolog or fragment thereof, or if the activity of a polypeptide encoded by a nucleic acid molecule comprising the nucleic acid SEQ ID NO. 36670, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 36670 or polypeptide SEQ ID NO. 36671, respectively, or a homolog or a fragment thereof, is increased or generated, or if the activity methylglyoxal synthase is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 20 to 51-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a methylglyoxal synthase, or if the activity of the polypeptide B0963, preferably represented by SEQ ID NO. 36671, or a homolog or fragment thereof, or if the activity of a polypeptide encoded by a nucleic acid molecule comprising the nucleic acid SEQ ID NO. 36670, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 36670 or polypeptide SEQ ID NO. 36671, respectively, or a homolog or a fragment thereof, is increased or generated, or if the activity methylglyoxal synthase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 58 to 241-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a methylglyoxal synthase, or if the activity of the polypeptide B0963, preferably represented by SEQ ID NO. 36671, or a homolog or fragment thereof, or if the activity of a polypeptide encoded by a nucleic acid molecule comprising the nucleic acid SEQ ID NO. 36670, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 36670 or polypeptide SEQ ID NO. 36671, respectively, or a homolog or a fragment thereof, is increased or generated, or if the activity methylglyoxal synthase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 46 to 453-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a phosphoanhydride phosphorylase, or if the activity of the polypeptide B0980, preferably represented by SEQ ID NO. 36810, or a homolog or fragment thereof, or if the activity of a polypeptide encoded by a nucleic acid molecule comprising the nucleic acid SEQ ID NO. 36809, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 36809 or polypeptide SEQ ID NO. 36810, respectively, or a homolog or a fragment thereof, is increased or generated, or if the activity phosphoanhydride phosphorylase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 32 to 89-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a b1003-protein, or if the activity of the polypeptide B1003, preferably represented by SEQ ID NO. 7942, or a homolog or fragment thereof, or if the activity of a polypeptide encoded by a nucleic acid molecule comprising the nucleic acid SEQ ID NO. 7941, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7941 or polypeptide SEQ ID NO. 7942, respectively, or a homolog or a fragment thereof, is increased or generated, or if the activity b1003-protein is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 51 to 123-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a b1003-protein, or if the activity of the polypeptide B1003, preferably represented by SEQ ID NO. 7942, or a homolog or fragment thereof, or if the activity of a polypeptide encoded by a nucleic acid molecule comprising the nucleic acid SEQ ID NO. 7941, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7941 or polypeptide SEQ ID NO. 7942, respectively, or a homolog or a fragment thereof, is increased or generated, or if the activity b1003-protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 60 to 317-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a b1003-protein, or if the activity of the polypeptide B1003, preferably represented by SEQ ID NO. 7942, or a homolog or fragment thereof, or if the activity of a polypeptide encoded by a nucleic acid molecule comprising the nucleic acid SEQ ID NO. 7941, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7941 or polypeptide SEQ ID NO. 7942, respectively, or a homolog or a fragment thereof, is increased or generated, or if the activity b1003-protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 72 to 728-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a b1003-protein, or if the activity of the polypeptide B1003, preferably represented by SEQ ID NO. 7942, or a homolog or fragment thereof, or if the activity of a polypeptide encoded by a nucleic acid molecule comprising the nucleic acid SEQ ID NO. 7941, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7941 or polypeptide SEQ ID NO. 7942, respectively, or a homolog or a fragment thereof, is increased or generated, or if the activity b1003-protein is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 33 to 327-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a b1003-protein, or if the activity of the polypeptide B1003, preferably represented by SEQ ID NO. 7942, or a homolog or fragment thereof, or if the activity of a polypeptide encoded by a nucleic acid molecule comprising the nucleic acid SEQ ID NO. 7941, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7941 or polypeptide SEQ ID NO. 7942, respectively, or a homolog or a fragment thereof, is increased or generated, or if the activity b1003-protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 119 to 664-percent is conferred as compared to a corresponding non-transformed wild type non-human organism.

Accordingly, in one embodiment, an increase of 3,4-dihydroxyphenylalanine (dopa) in a non-human organism, as compared to a corresponding non-transformed wild type non-human organism, is conferred in the process of the invention, if the activity of a polypeptide showing the activity of a b1003-protein, or if the activity of the polypeptide B1003, preferably represented by SEQ ID NO. 7942, or a homolog or fragment thereof, or if the activity of a polypeptide encoded by a nucleic acid molecule comprising the nucleic acid SEQ ID NO. 7941, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7941 or polypeptide SEQ ID NO. 7942, respectively, or a homolog or a fragment thereof, is increased or generated, or if the activity b1003-protein is increased or generated in a non-human organism, like a microorganism or a plant cell, plant or part thereof, especially with non-targeted localization, whereby the respective line discloses in column 7 3,4-dihydroxyphenylalanine (dopa). For example, an increase of the 3,4-dihydroxyphenylalanine (dopa) of at least 1 percent, particularly in a range of 36 to 327-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a b1003-protein, or if the activity of the polypeptide B1003, preferably represented by SEQ ID NO. 7942, or a homolog or fragment thereof, or if the activity of a polypeptide encoded by a nucleic acid molecule comprising the nucleic acid SEQ ID NO. 7941, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7941 or polypeptide SEQ ID NO. 7942, respectively, or a homolog or a fragment thereof, is increased or generated, or if the activity b1003-protein is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 34 to 935-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a lipoprotein precursor, or if the activity of the polypeptide B1023, preferably represented by SEQ ID NO. 36881, or a homolog or fragment thereof, or if the activity of a polypeptide encoded by a nucleic acid molecule comprising the nucleic acid SEQ ID NO. 36880, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 36880 or polypeptide SEQ ID NO. 36881, respectively, or a homolog or a fragment thereof, is increased or generated, or if the activity lipoprotein precursor is increased or generated in a non-human organism, like a microorganism or a plant cell, plant or part thereof, especially with non-targeted localization, whereby the respective line discloses in column 7 citrulline. For example, an increase of the citrulline of at least 1 percent, particularly in a range of 44 to 123-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a lipoprotein precursor, or if the activity of the polypeptide B1023, preferably represented by SEQ ID NO. 36881, or a homolog or fragment thereof, or if the activity of a polypeptide encoded by a nucleic acid molecule comprising the nucleic acid SEQ ID NO. 36880, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 36880 or polypeptide SEQ ID NO. 36881, respectively, or a homolog or a fragment thereof, is increased or generated, or if the activity lipoprotein precursor is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 38 to 138-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a b1024-protein, or if the activity of the polypeptide B1024, preferably represented by SEQ ID NO. 36908, or a homolog or fragment thereof, or if the activity of a polypeptide encoded by a nucleic acid molecule comprising the nucleic acid SEQ ID NO. 36907, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 36907 or polypeptide SEQ ID NO. 36908, respectively, or a homolog or a fragment thereof, is increased or generated, or if the activity b1024-protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 63 to 105-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a multidrug resistance protein, or if the activity of the polypeptide B1065, preferably represented by SEQ ID NO. 76033, or a homolog or fragment thereof, or if the activity of a polypeptide encoded by a nucleic acid molecule comprising the nucleic acid SEQ ID NO. 76032, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 76032 or polypeptide SEQ ID NO. 76033, respectively, or a homolog or a fragment thereof, is increased or generated, or if the activity multidrug resistance protein is increased or generated in a non-human organism, like a microorganism or a plant cell, plant or part thereof, especially with plastidic localization, whereby the respective line discloses in column 7 aspartate. For example, an increase of the aspartate of at least 1 percent, particularly in a range of 53 to 132-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a multidrug resistance protein, or if the activity of the polypeptide B1065, preferably represented by SEQ ID NO. 76033, or a homolog or fragment thereof, or if the activity of a polypeptide encoded by a nucleic acid molecule comprising the nucleic acid SEQ ID NO. 76032, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 76032 or polypeptide SEQ ID NO. 76033, respectively, or a homolog or a fragment thereof, is increased or generated, or if the activity multidrug resistance protein is increased or generated in a non-human organism, like a microorganism or a plant cell, plant or part thereof, especially with plastidic localization, whereby the respective line discloses in column 7 tyrosine. For example, an increase of the tyrosine of at least 1 percent, particularly in a range of 34 to 45-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a b1137-protein, or if the activity of the polypeptide B1137, preferably represented by SEQ ID NO. 37391, or a homolog or fragment thereof, or if the activity of a polypeptide encoded by a nucleic acid molecule comprising the nucleic acid SEQ ID NO. 37390, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 37390 or polypeptide SEQ ID NO. 37391, respectively, or a homolog or a fragment thereof, is increased or generated, or if the activity b1137-protein is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 27 to 91-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a b1155-protein, or if the activity of the polypeptide B1155, preferably represented by SEQ ID NO. 76069, or a homolog or fragment thereof, or if the activity of a polypeptide encoded by a nucleic acid molecule comprising the nucleic acid SEQ ID NO. 76068, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 76068 or polypeptide SEQ ID NO. 76069, respectively, or a homolog or a fragment thereof, is increased or generated, or if the activity b1155-protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 34 to 71-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a b1163-protein, or if the activity of the polypeptide B1163, preferably represented by SEQ ID NO. 37395, or a homolog or fragment thereof, or if the activity of a polypeptide encoded by a nucleic acid molecule comprising the nucleic acid SEQ ID NO. 37394, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 37394 or polypeptide SEQ ID NO. 37395, respectively, or a homolog or a fragment thereof, is increased or generated, or if the activity b1163-protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 30 to 38-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a b1163-protein, or if the activity of the polypeptide B1163, preferably represented by SEQ ID NO. 37395, or a homolog or fragment thereof, or if the activity of a polypeptide encoded by a nucleic acid molecule comprising the nucleic acid SEQ ID NO. 37394, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 37394 or polypeptide SEQ ID NO. 37395, respectively, or a homolog or a fragment thereof, is increased or generated, or if the activity b1163-protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 39 to 45-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a b1179-protein, or if the activity of the polypeptide B1179, preferably represented by SEQ ID NO. 76088, or a homolog or fragment thereof, or if the activity of a polypeptide encoded by a nucleic acid molecule comprising the nucleic acid SEQ ID NO. 76087, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 76087 or polypeptide SEQ ID NO. 76088, respectively, or a homolog or a fragment thereof, is increased or generated, or if the activity b1179-protein is increased or generated in a non-human organism, like a microorganism or a plant cell, plant or part thereof, especially with plastidic localization, whereby the respective line discloses in column 7 citrulline. For example, an increase of the citrulline of at least 1 percent, particularly in a range of 35 to 47-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a sodium/proton antiporter, or if the activity of the polypeptide B1186, preferably represented by SEQ ID NO. 37401, or a homolog or fragment thereof, or if the activity of a polypeptide encoded by a nucleic acid molecule comprising the nucleic acid SEQ ID NO. 37400, preferably the coding region thereof, or a homolog or fragment thereof, e.g. derived from Escherichia coli K12, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 37400 or polypeptide SEQ ID NO. 37401, respectively, or a homolog or a fragment thereof, is increased or generated, or if the activity sodium/proton antiporter is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 28 to 76-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a sodium/proton antiporter, or if the activity of the polypeptide B1186, preferably represented by SEQ ID NO. 37401, or a homolog or fragment thereof, or if the activity of a polypeptide encoded by a nucleic acid molecule comprising the nucleic acid SEQ ID NO. 37400, preferably the coding region thereof, or a homolog or fragment thereof, e.g. derived from Escherichia coli K12, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 37400 or polypeptide SEQ ID NO. 37401, respectively, or a homolog or a fragment thereof, is increased or generated, or if the activity sodium/proton antiporter is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 46 to 113-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a b1205-protein, or if the activity of the polypeptide B1205, preferably represented by SEQ ID NO. 76139, or a homolog or fragment thereof, or if the activity of a polypeptide encoded by a nucleic acid molecule comprising the nucleic acid SEQ ID NO. 76138, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 76138 or polypeptide SEQ ID NO. 76139, respectively, or a homolog or a fragment thereof, is increased or generated, or if the activity b1205-protein is increased or generated in a non-human organism, like a microorganism or a plant cell, plant or part thereof, especially with non-targeted localization, whereby the respective line discloses in column 7 citrulline. For example, an increase of the citrulline of at least 1 percent, particularly in a range of 35 to 77-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a b1229protein, or if the activity of the polypeptide B1229, preferably represented by SEQ ID NO. 76152, or a homolog or fragment thereof, or if the activity of a polypeptide encoded by a nucleic acid molecule comprising the nucleic acid SEQ ID NO. 76151, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 76151 or polypeptide SEQ ID NO. 76152, respectively, or a homolog or a fragment thereof, is increased or generated, or if the activity b1229-protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 35 to 44-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a membrane protein, or if the activity of the polypeptide B1255, preferably represented by SEQ ID NO. 37484, or a homolog or fragment thereof, or if the activity of a polypeptide encoded by a nucleic acid molecule comprising the nucleic acid SEQ ID NO. 37483, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 37483 or polypeptide SEQ ID NO. 37484, respectively, or a homolog or a fragment thereof, is increased or generated, or if the activity membrane protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 52 to 124-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a b1259-protein, or if the activity of the polypeptide B1259, preferably represented by SEQ ID NO. 37504, or a homolog or fragment thereof, or if the activity of a polypeptide encoded by a nucleic acid molecule comprising the nucleic acid SEQ ID NO. 37503, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 37503 or polypeptide SEQ ID NO. 37504, respectively, or a homolog or a fragment thereof, is increased or generated, or if the activity b1259-protein is increased or generated in a non-human organism, like a microorganism or a plant cell, plant or part thereof, especially with non-targeted localization, whereby the respective line discloses in column 7 citrulline. For example, an increase of the citrulline of at least 1 percent, particularly in a range of 32 to 142-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a b1259-protein, or if the activity of the polypeptide B1259, preferably represented by SEQ ID NO. 37504, or a homolog or fragment thereof, or if the activity of a polypeptide encoded by a nucleic acid molecule comprising the nucleic acid SEQ ID NO. 37503, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 37503 or polypeptide SEQ ID NO. 37504, respectively, or a homolog or a fragment thereof, is increased or generated, or if the activity b1259-protein is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 38 to 91-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a tryptophan biosynthesis protein, or if the activity of the polypeptide B1262, preferably represented by SEQ ID NO. 76158, or a homolog or fragment thereof, or if the activity of a polypeptide encoded by a nucleic acid molecule comprising the nucleic acid SEQ ID NO. 76157, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 76157 or polypeptide SEQ ID NO. 76158, respectively, or a homolog or a fragment thereof, is increased or generated, or if the activity tryptophan biosynthesis protein is increased or generated in a non-human organism, like a microorganism or a plant cell, plant or part thereof, especially with plastidic localization, whereby the respective line discloses in column 7 tyrosine. For example, an increase of the tyrosine of at least 1 percent, particularly in a range of 27 to 79-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a anthranilate synthase component I, or if the activity of the polypeptide B1264, preferably represented by SEQ ID NO. 76241, or a homolog or fragment thereof, or if the activity of a polypeptide encoded by a nucleic acid molecule comprising the nucleic acid SEQ ID NO. 76240, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 76240 or polypeptide SEQ ID NO. 76241, respectively, or a homolog or a fragment thereof, is increased or generated, or if the activity anthranilate synthase component I is increased or generated in a non-human organism, like a microorganism or a plant cell, plant or part thereof, especially with plastidic localization, whereby the respective line discloses in column 7 phenylalanine. For example, an increase of the phenylalanine of at least 1 percent, particularly in a range of 33 to 47-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a b1280-protein, or if the activity of the polypeptide B1280, preferably represented by SEQ ID NO. 37574, or a homolog or fragment thereof, or if the activity of a polypeptide encoded by a nucleic acid molecule comprising the nucleic acid SEQ ID NO. 37573, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 37573 or polypeptide SEQ ID NO. 37574, respectively, or a homolog or a fragment thereof, is increased or generated, or if the activity b1280-protein is increased or generated in a non-human organism, like a microorganism or a plant cell, plant or part thereof, especially with non-targeted localization, whereby the respective line discloses in column 7 glycine. For example, an increase of the glycine of at least 1 percent, particularly in a range of 38 to 169-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a b1280-protein, or if the activity of the polypeptide B1280, preferably represented by SEQ ID NO. 37574, or a homolog or fragment thereof, or if the activity of a polypeptide encoded by a nucleic acid molecule comprising the nucleic acid SEQ ID NO. 37573, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 37573 or polypeptide SEQ ID NO. 37574, respectively, or a homolog or a fragment thereof, is increased or generated, or if the activity b1280-protein is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 37 to 48-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a b1280-protein, or if the activity of the polypeptide B1280, preferably represented by SEQ ID NO. 37574, or a homolog or fragment thereof, or if the activity of a polypeptide encoded by a nucleic acid molecule comprising the nucleic acid SEQ ID NO. 37573, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 37573 or polypeptide SEQ ID NO. 37574, respectively, or a homolog or a fragment thereof, is increased or generated, or if the activity b1280-protein is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 38 to 86-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a glutamine synthetase, or if the activity of the polypeptide B1297, preferably represented by SEQ ID NO. 37659, or a homolog or fragment thereof, or if the activity of a polypeptide encoded by a nucleic acid molecule comprising the nucleic acid SEQ ID NO. 37658, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 37658 or polypeptide SEQ ID NO. 37659, respectively, or a homolog or a fragment thereof, is increased or generated, or if the activity glutamine synthetase is increased or generated in a non-human organism, like a microorganism or a plant cell, plant or part thereof, especially with plastidic localization, whereby the respective line discloses in column 7 alanine. For example, an increase of the alanine of at least 1 percent, particularly in a range of 62 to 345-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a glutamine synthetase, or if the activity of the polypeptide B1297, preferably represented by SEQ ID NO. 37659, or a homolog or fragment thereof, or if the activity of a polypeptide encoded by a nucleic acid molecule comprising the nucleic acid SEQ ID NO. 37658, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 37658 or polypeptide SEQ ID NO. 37659, respectively, or a homolog or a fragment thereof, is increased or generated, or if the activity glutamine synthetase is increased or generated in a non-human organism, like a microorganism or a plant cell, plant or part thereof, especially with plastidic localization, whereby the respective line discloses in column 7 glycine. For example, an increase of the glycine of at least 1 percent, particularly in a range of 26 to 507-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a aldehyde dehydrogenase, or if the activity of the polypeptide B1300, preferably represented by SEQ ID NO. 37808, or a homolog or fragment thereof, or if the activity of a polypeptide encoded by a nucleic acid molecule comprising the nucleic acid SEQ ID NO. 37807, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 37807 or polypeptide SEQ ID NO. 37808, respectively, or a homolog or a fragment thereof, is increased or generated, or if the activity aldehyde dehydrogenase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 52 to 109-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a aldehyde dehydrogenase, or if the activity of the polypeptide B1300, preferably represented by SEQ ID NO. 37808, or a homolog or fragment thereof, or if the activity of a polypeptide encoded by a nucleic acid molecule comprising the nucleic acid SEQ ID NO. 37807, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 37807 or polypeptide SEQ ID NO. 37808, respectively, or a homolog or a fragment thereof, is increased or generated, or if the activity aldehyde dehydrogenase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 59 to 115-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a ABC transporter substrate-binding protein, or if the activity of the polypeptide B1310, preferably represented by SEQ ID NO. 76868, or a homolog or fragment thereof, or if the activity of a polypeptide encoded by a nucleic acid molecule comprising the nucleic acid SEQ ID NO. 76867, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 76867 or polypeptide SEQ ID NO. 76868, respectively, or a homolog or a fragment thereof, is increased or generated, or if the activity ABC transporter substrate-binding protein is increased or generated in a non-human organism, like a microorganism or a plant cell, plant or part thereof, especially with non-targeted localization, whereby the respective line discloses in column 7 citrulline. For example, an increase of the citrulline of at least 1 percent, particularly in a range of 33 to 51-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a b1330-protein, or if the activity of the polypeptide B1330, preferably represented by SEQ ID NO. 38227, or a homolog or fragment thereof, or if the activity of a polypeptide encoded by a nucleic acid molecule comprising the nucleic acid SEQ ID NO. 38226, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 38226 or polypeptide SEQ ID NO. 38227, respectively, or a homolog or a fragment thereof, is increased or generated, or if the activity b1330-protein is increased or generated in a non-human organism, like a microorganism or a plant cell, plant or part thereof, especially with non-targeted localization, whereby the respective line discloses in column 7 citrulline. For example, an increase of the citrulline of at least 1 percent, particularly in a range of 33 to 162-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a ATP-dependent RNA helicase, or if the activity of the polypeptide B1343, preferably represented by SEQ ID NO. 76891, or a homolog or fragment thereof, or if the activity of a polypeptide encoded by a nucleic acid molecule comprising the nucleic acid SEQ ID NO. 76890, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 76890 or polypeptide SEQ ID NO. 76891, respectively, or a homolog or a fragment thereof, is increased or generated, or if the activity ATP-dependent RNA helicase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 27 to 100-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a restriction alleviation protein, or if the activity of the polypeptide B1348, preferably represented by SEQ ID NO. 77769, or a homolog or fragment thereof, or if the activity of a polypeptide encoded by a nucleic acid molecule comprising the nucleic acid SEQ ID NO. 77768, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 77768 or polypeptide SEQ ID NO. 77769, respectively, or a homolog or a fragment thereof, is increased or generated, or if the activity restriction alleviation protein is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 23 to 36-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a restriction alleviation protein, or if the activity of the polypeptide B1348, preferably represented by SEQ ID NO. 77769, or a homolog or fragment thereof, or if the activity of a polypeptide encoded by a nucleic acid molecule comprising the nucleic acid SEQ ID NO. 77768, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 77768 or polypeptide SEQ ID NO. 77769, respectively, or a homolog or a fragment thereof, is increased or generated, or if the activity restriction alleviation protein is increased or generated in a non-human organism, like a microorganism or a plant cell, plant or part thereof, especially with non-targeted localization, whereby the respective line discloses in column 7 glycine. For example, an increase of the glycine of at least 1 percent, particularly in a range of 47 to 134-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a lipoprotein precursor, or if the activity of the polypeptide B1431, preferably represented by SEQ ID NO. 38267, or a homolog or fragment thereof, or if the activity of a polypeptide encoded by a nucleic acid molecule comprising the nucleic acid SEQ ID NO. 38266, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 38266 or polypeptide SEQ ID NO. 38267, respectively, or a homolog or a fragment thereof, is increased or generated, or if the activity lipoprotein precursor is increased or generated in a non-human organism, like a microorganism or a plant cell, plant or part thereof, especially with plastidic localization, whereby the respective line discloses in column 7 5-oxoproline. For example, an increase of the 5-oxoproline of at least 1 percent, particularly in a range of 37 to 46-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a b1445-protein, or if the activity of the polypeptide B1445, preferably represented by SEQ ID NO. 38290, or a homolog or fragment thereof, or if the activity of a polypeptide encoded by a nucleic acid molecule comprising the nucleic acid SEQ ID NO. 38289, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 38289 or polypeptide SEQ ID NO. 38290, respectively, or a homolog or a fragment thereof, is increased or generated, or if the activity b1445-protein is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 19 to 46-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a b1445-protein, or if the activity of the polypeptide B1445, preferably represented by SEQ ID NO. 38290, or a homolog or fragment thereof, or if the activity of a polypeptide encoded by a nucleic acid molecule comprising the nucleic acid SEQ ID NO. 38289, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 38289 or polypeptide SEQ ID NO. 38290, respectively, or a homolog or a fragment thereof, is increased or generated, or if the activity b1445-protein is increased or generated in a non-human organism, like a microorganism or a plant cell, plant or part thereof, especially with non-targeted localization, whereby the respective line discloses in column 7 citrulline. For example, an increase of the citrulline of at least 1 percent, particularly in a range of 32 to 96-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a b1445-protein, or if the activity of the polypeptide B1445, preferably represented by SEQ ID NO. 38290, or a homolog or fragment thereof, or if the activity of a polypeptide encoded by a nucleic acid molecule comprising the nucleic acid SEQ ID NO. 38289, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 38289 or polypeptide SEQ ID NO. 38290, respectively, or a homolog or a fragment thereof, is increased or generated, or if the activity b1445-protein is increased or generated in a non-human organism, like a microorganism or a plant cell, plant or part thereof, especially with non-targeted localization, whereby the respective line discloses in column 7 glycine. For example, an increase of the glycine of at least 1 percent, particularly in a range of 37 to 82-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a b1445-protein, or if the activity of the polypeptide B1445, preferably represented by SEQ ID NO. 38290, or a homolog or fragment thereof, or if the activity of a polypeptide encoded by a nucleic acid molecule comprising the nucleic acid SEQ ID NO. 38289, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 38289 or polypeptide SEQ ID NO. 38290, respectively, or a homolog or a fragment thereof, is increased or generated, or if the activity b1445-protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 34 to 58-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a b1445-protein, or if the activity of the polypeptide B1445, preferably represented by SEQ ID NO. 38290, or a homolog or fragment thereof, or if the activity of a polypeptide encoded by a nucleic acid molecule comprising the nucleic acid SEQ ID NO. 38289, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 38289 or polypeptide SEQ ID NO. 38290, respectively, or a homolog or a fragment thereof, is increased or generated, or if the activity b1445-protein is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 37 to 78-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a NADP-dependent malic enzyme, or if the activity of the polypeptide B1479, preferably represented by SEQ ID NO. 77775, or a homolog or fragment thereof, or if the activity of a polypeptide encoded by a nucleic acid molecule comprising the nucleic acid SEQ ID NO. 77774, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 77774 or polypeptide SEQ ID NO. 77775, respectively, or a homolog or a fragment thereof, is increased or generated, or if the activity NADP-dependent malic enzyme is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 27 to 184-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a NADP-dependent malic enzyme, or if the activity of the polypeptide B1479, preferably represented by SEQ ID NO. 77775, or a homolog or fragment thereof, or if the activity of a polypeptide encoded by a nucleic acid molecule comprising the nucleic acid SEQ ID NO. 77774, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 77774 or polypeptide SEQ ID NO. 77775, respectively, or a homolog or a fragment thereof, is increased or generated, or if the activity NADP-dependent malic enzyme is increased or generated in a non-human organism, like a microorganism or a plant cell, plant or part thereof, especially with non-targeted localization, whereby the respective line discloses in column 7 glycine. For example, an increase of the glycine of at least 1 percent, particularly in a range of 45 to 211-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a NADP-dependent malic enzyme, or if the activity of the polypeptide B1479, preferably represented by SEQ ID NO. 77775, or a homolog or fragment thereof, or if the activity of a polypeptide encoded by a nucleic acid molecule comprising the nucleic acid SEQ ID NO. 77774, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 77774 or polypeptide SEQ ID NO. 77775, respectively, or a homolog or a fragment thereof, is increased or generated, or if the activity NADP-dependent malic enzyme is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 35 to 155-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a NADP-dependent malic enzyme, or if the activity of the polypeptide B1479, preferably represented by SEQ ID NO. 77775, or a homolog or fragment thereof, or if the activity of a polypeptide encoded by a nucleic acid molecule comprising the nucleic acid SEQ ID NO. 77774, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 77774 or polypeptide SEQ ID NO. 77775, respectively, or a homolog or a fragment thereof, is increased or generated, or if the activity NADP-dependent malic enzyme is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 29 to 287-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a NADP-dependent malic enzyme, or if the activity of the polypeptide B1479, preferably represented by SEQ ID NO. 77775, or a homolog or fragment thereof, or if the activity of a polypeptide encoded by a nucleic acid molecule comprising the nucleic acid SEQ ID NO. 77774, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 77774 or polypeptide SEQ ID NO. 77775, respectively, or a homolog or a fragment thereof, is increased or generated, or if the activity NADP-dependent malic enzyme is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 32 to 107-percent is conferred as compared to a corresponding non-transformed wild type non-human organism.

Accordingly, in one embodiment, an increase of asparagine in a non-human organism, as compared to a corresponding non-transformed wild type non-human organism, is conferred in the process of the invention, if the activity of a polypeptide showing the activity of a b1522-protein, or if the activity of the polypeptide B1522, preferably represented by SEQ ID NO. 7948, or a homolog or fragment thereof, or if the activity of a polypeptide encoded by a nucleic acid molecule comprising the nucleic acid SEQ ID NO. 7947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7947 or polypeptide SEQ ID NO. 7948, respectively, or a homolog or a fragment thereof, is increased or generated, or if the activity b1522-protein is increased or generated in a non-human organism, like a microorganism or a plant cell, plant or part thereof, especially with non-targeted localization, whereby the respective line discloses in column 7 asparagine. For example, an increase of the asparagine of at least 1 percent, particularly in a range of 42 to 1317-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a b1522-protein, or if the activity of the polypeptide B1522, preferably represented by SEQ ID NO. 7948, or a homolog or fragment thereof, or if the activity of a polypeptide encoded by a nucleic acid molecule comprising the nucleic acid SEQ ID NO. 7947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7947 or polypeptide SEQ ID NO. 7948, respectively, or a homolog or a fragment thereof, is increased or generated, or if the activity b1522-protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 66 to 168-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a b1522-protein, or if the activity of the polypeptide B1522, preferably represented by SEQ ID NO. 7948, or a homolog or fragment thereof, or if the activity of a polypeptide encoded by a nucleic acid molecule comprising the nucleic acid SEQ ID NO. 7947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7947 or polypeptide SEQ ID NO. 7948, respectively, or a homolog or a fragment thereof, is increased or generated, or if the activity b1522-protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 34 to 1168-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a b1522-protein, or if the activity of the polypeptide B1522, preferably represented by SEQ ID NO. 7948, or a homolog or fragment thereof, or if the activity of a polypeptide encoded by a nucleic acid molecule comprising the nucleic acid SEQ ID NO. 7947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7947 or polypeptide SEQ ID NO. 7948, respectively, or a homolog or a fragment thereof, is increased or generated, or if the activity b1522-protein is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 23 to 109-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a b1522-protein, or if the activity of the polypeptide B1522, preferably represented by SEQ ID NO. 7948, or a homolog or fragment thereof, or if the activity of a polypeptide encoded by a nucleic acid molecule comprising the nucleic acid SEQ ID NO. 7947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7947 or polypeptide SEQ ID NO. 7948, respectively, or a homolog or a fragment thereof, is increased or generated, or if the activity b1522-protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 38 to 627-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a b1522-protein, or if the activity of the polypeptide B1522, preferably represented by SEQ ID NO. 7948, or a homolog or fragment thereof, or if the activity of a polypeptide encoded by a nucleic acid molecule comprising the nucleic acid SEQ ID NO. 7947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 7947 or polypeptide SEQ ID NO. 7948, respectively, or a homolog or a fragment thereof, is increased or generated, or if the activity b1522-protein is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 47 to 133-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a b1583-protein, or if the activity of the polypeptide B1583, preferably represented by SEQ ID NO. 78139, or a homolog or fragment thereof, or if the activity of a polypeptide encoded by a nucleic acid molecule comprising the nucleic acid SEQ ID NO. 78138, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 78138 or polypeptide SEQ ID NO. 78139, respectively, or a homolog or a fragment thereof, is increased or generated, or if the activity b1583-protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 51 to 184-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a Fe—S subunit of oxidoreductase, or if the activity of the polypeptide B1589, preferably represented by SEQ ID NO. 78155, or a homolog or fragment thereof, or if the activity of a polypeptide encoded by a nucleic acid molecule comprising the nucleic acid SEQ ID NO. 78154, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 78154 or polypeptide SEQ ID NO. 78155, respectively, or a homolog or a fragment thereof, is increased or generated, or if the activity Fe—S subunit of oxidoreductase is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 52 to 74-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a acid shock protein, or if the activity of the polypeptide B1597, preferably represented by SEQ ID NO. 38301, or a homolog or fragment thereof, or if the activity of a polypeptide encoded by a nucleic acid molecule comprising the nucleic acid SEQ ID NO. 38300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 38300 or polypeptide SEQ ID NO. 38301, respectively, or a homolog or a fragment thereof, is increased or generated, or if the activity acid shock protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 65 to 190-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a acid shock protein, or if the activity of the polypeptide B1597, preferably represented by SEQ ID NO. 38301, or a homolog or fragment thereof, or if the activity of a polypeptide encoded by a nucleic acid molecule comprising the nucleic acid SEQ ID NO. 38300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 38300 or polypeptide SEQ ID NO. 38301, respectively, or a homolog or a fragment thereof, is increased or generated, or if the activity acid shock protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 64 to 143-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a methylglyoxalase, or if the activity of the polypeptide B1651, preferably represented by SEQ ID NO. 78266, or a homolog or fragment thereof, or if the activity of a polypeptide encoded by a nucleic acid molecule comprising the nucleic acid SEQ ID NO. 78265, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 78265 or polypeptide SEQ ID NO. 78266, respectively, or a homolog or a fragment thereof, is increased or generated, or if the activity methylglyoxalase is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 19 to 39-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a b1670-protein, or if the activity of the polypeptide B1670, preferably represented by SEQ ID NO. 78754, or a homolog or fragment thereof, or if the activity of a polypeptide encoded by a nucleic acid molecule comprising the nucleic acid SEQ ID NO. 78753, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 78753 or polypeptide SEQ ID NO. 78754, respectively, or a homolog or a fragment thereof, is increased or generated, or if the activity b1670-protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 58 to 193-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a b1672-protein, or if the activity of the polypeptide B1672, preferably represented by SEQ ID NO. 78772, or a homolog or fragment thereof, or if the activity of a polypeptide encoded by a nucleic acid molecule comprising the nucleic acid SEQ ID NO. 78771, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 78771 or polypeptide SEQ ID NO. 78772, respectively, or a homolog or a fragment thereof, is increased or generated, or if the activity b1672-protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 59 to 111-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a arginine N-succinyltransferase, or if the activity of the polypeptide B1747, preferably represented by SEQ ID NO. 78780, or a homolog or fragment thereof, or if the activity of a polypeptide encoded by a nucleic acid molecule comprising the nucleic acid SEQ ID NO. 78779, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 78779 or polypeptide SEQ ID NO. 78780, respectively, or a homolog or a fragment thereof, is increased or generated, or if the activity arginine N-succinyltransferase is increased or generated in a non-human organism, like a microorganism or a plant cell, plant or part thereof, especially with plastidic localization, whereby the respective line discloses in column 7 alanine. For example, an increase of the alanine of at least 1 percent, particularly in a range of 70 to 193-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a ABC transporter permease protein, or if the activity of the polypeptide B1755, preferably represented by SEQ ID NO. 78853, or a homolog or fragment thereof, or if the activity of a polypeptide encoded by a nucleic acid molecule comprising the nucleic acid SEQ ID NO. 78852, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 78852 or polypeptide SEQ ID NO. 78853, respectively, or a homolog or a fragment thereof, is increased or generated, or if the activity ABC transporter permease protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 52 to 103-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a ABC transporter permease protein, or if the activity of the polypeptide B1755, preferably represented by SEQ ID NO. 78853, or a homolog or fragment thereof, or if the activity of a polypeptide encoded by a nucleic acid molecule comprising the nucleic acid SEQ ID NO. 78852, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 78852 or polypeptide SEQ ID NO. 78853, respectively, or a homolog or a fragment thereof, is increased or generated, or if the activity ABC transporter permease protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 51 to 120-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a transport protein, or if the activity of the polypeptide B1791, preferably represented by SEQ ID NO. 78884, or a homolog or fragment thereof, or if the activity of a polypeptide encoded by a nucleic acid molecule comprising the nucleic acid SEQ ID NO. 78883, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 78883 or polypeptide SEQ ID NO. 78884, respectively, or a homolog or a fragment thereof, is increased or generated, or if the activity transport protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 54 to 114-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a b1837-protein, or if the activity of the polypeptide B1837, preferably represented by SEQ ID NO. 78954, or a homolog or fragment thereof, or if the activity of a polypeptide encoded by a nucleic acid molecule comprising the nucleic acid SEQ ID NO. 78953, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 78953 or polypeptide SEQ ID NO. 78954, respectively, or a homolog or a fragment thereof, is increased or generated, or if the activity b1837-protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 51 to 69-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a serine/threonine-protein phosphatase, or if the activity of the polypeptide B1838, preferably represented by SEQ ID NO. 8317, or a homolog or fragment thereof, or if the activity of a polypeptide encoded by a nucleic acid molecule comprising the nucleic acid SEQ ID NO. 8316, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 8316 or polypeptide SEQ ID NO. 8317, respectively, or a homolog or a fragment thereof, is increased or generated, or if the activity serine/threonine-protein phosphatase is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 70 to 104-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a protease, or if the activity of the polypeptide B1845, preferably represented by SEQ ID NO. 38574, or a homolog or fragment thereof, or if the activity of a polypeptide encoded by a nucleic acid molecule comprising the nucleic acid SEQ ID NO. 38573, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 38573 or polypeptide SEQ ID NO. 38574, respectively, or a homolog or a fragment thereof, is increased or generated, or if the activity protease is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 57 to 199-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a protease, or if the activity of the polypeptide B1845, preferably represented by SEQ ID NO. 38574, or a homolog or fragment thereof, or if the activity of a polypeptide encoded by a nucleic acid molecule comprising the nucleic acid SEQ ID NO. 38573, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 38573 or polypeptide SEQ ID NO. 38574, respectively, or a homolog or a fragment thereof, is increased or generated, or if the activity protease is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 49 to 125-percent is conferred as compared to a corresponding non-transformed wild type non-human organism.

Accordingly, in one embodiment, an increase of asparagine in a non-human organism, as compared to a corresponding non-transformed wild type non-human organism, is conferred in the process of the invention, if the activity of a polypeptide showing the activity of a pyruvate kinase, or if the activity of the polypeptide B1854, preferably represented by SEQ ID NO. 8364, or a homolog or fragment thereof, or if the activity of a polypeptide encoded by a nucleic acid molecule comprising the nucleic acid SEQ ID NO. 8363, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 8363 or polypeptide SEQ ID NO. 8364, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line discloses in column 7 asparagine. For example, an increase of the asparagine of at least 1 percent, particularly in a range of 32 to 157-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a cytochrome ctype protein, or if the activity of the polypeptide B1873, preferably represented by SEQ ID NO. 78971, or a homolog or fragment thereof, or if the activity of a polypeptide encoded by a nucleic acid molecule comprising the nucleic acid SEQ ID NO. 78970, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 78970 or polypeptide SEQ ID NO. 78971, respectively, or a homolog or a fragment thereof, is increased or generated, or if the activity cytochrome c-type protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 58 to 217-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a trehalosephosphatase, or if the activity of the polypeptide B1897, preferably represented by SEQ ID NO. 79057, or a homolog or fragment thereof, or if the activity of a polypeptide encoded by a nucleic acid molecule comprising the nucleic acid SEQ ID NO. 79056, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 79056 or polypeptide SEQ ID NO. 79057, respectively, or a homolog or a fragment thereof, is increased or generated, or if the activity trehalose-phosphatase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 28 to 196-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a trehalosephosphatase, or if the activity of the polypeptide B1897, preferably represented by SEQ ID NO. 79057, or a homolog or fragment thereof, or if the activity of a polypeptide encoded by a nucleic acid molecule comprising the nucleic acid SEQ ID NO. 79056, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 79056 or polypeptide SEQ ID NO. 79057, respectively, or a homolog or a fragment thereof, is increased or generated, or if the activity trehalose-phosphatase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 36 to 211-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a b1898-protein, or if the activity of the polypeptide B1898, preferably represented by SEQ ID NO. 38768, or a homolog or fragment thereof, or if the activity of a polypeptide encoded by a nucleic acid molecule comprising the nucleic acid SEQ ID NO. 38767, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 38767 or polypeptide SEQ ID NO. 38768, respectively, or a homolog or a fragment thereof, is increased or generated, or if the activity b1898-protein is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 28 to 106-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a b1930-protein, or if the activity of the polypeptide B1930, preferably represented by SEQ ID NO. 79182, or a homolog or fragment thereof, or if the activity of a polypeptide encoded by a nucleic acid molecule comprising the nucleic acid SEQ ID NO. 79181, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 79181 or polypeptide SEQ ID NO. 79182, respectively, or a homolog or a fragment thereof, is increased or generated, or if the activity b1930-protein is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 42 to 113-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a transport protein, or if the activity of the polypeptide B1981, preferably represented by SEQ ID NO. 38900, or a homolog or fragment thereof, or if the activity of a polypeptide encoded by a nucleic acid molecule comprising the nucleic acid SEQ ID NO. 38899, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 38899 or polypeptide SEQ ID NO. 38900, respectively, or a homolog or a fragment thereof, is increased or generated, or if the activity transport protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 66 to 90-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a O-antigen chain length determinant, or if the activity of the polypeptide B2027, preferably represented by SEQ ID NO. 79218, or a homolog or fragment thereof, or if the activity of a polypeptide encoded by a nucleic acid molecule comprising the nucleic acid SEQ ID NO. 79217, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 79217 or polypeptide SEQ ID NO. 79218, respectively, or a homolog or a fragment thereof, is increased or generated, or if the activity O-antigen chain length determinant is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 56 to 437-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a b2032-protein, or if the activity of the polypeptide B2032, preferably represented by SEQ ID NO. 8921, or a homolog or fragment thereof, or if the activity of a polypeptide encoded by a nucleic acid molecule comprising the nucleic acid SEQ ID NO. 8920, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 8920 or polypeptide SEQ ID NO. 8921, respectively, or a homolog or a fragment thereof, is increased or generated, or if the activity b2032-protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 64 to 210-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a transport protein, or if the activity of the polypeptide B2063, preferably represented by SEQ ID NO. 38948, or a homolog or fragment thereof, or if the activity of a polypeptide encoded by a nucleic acid molecule comprising the nucleic acid SEQ ID NO. 38947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 38947 or polypeptide SEQ ID NO. 38948, respectively, or a homolog or a fragment thereof, is increased or generated, or if the activity transport protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 56 to 184-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a transport protein, or if the activity of the polypeptide B2063, preferably represented by SEQ ID NO. 38948, or a homolog or fragment thereof, or if the activity of a polypeptide encoded by a nucleic acid molecule comprising the nucleic acid SEQ ID NO. 38947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 38947 or polypeptide SEQ ID NO. 38948, respectively, or a homolog or a fragment thereof, is increased or generated, or if the activity transport protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 32 to 1359-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a transport protein, or if the activity of the polypeptide B2063, preferably represented by SEQ ID NO. 38948, or a homolog or fragment thereof, or if the activity of a polypeptide encoded by a nucleic acid molecule comprising the nucleic acid SEQ ID NO. 38947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 38947 or polypeptide SEQ ID NO. 38948, respectively, or a homolog or a fragment thereof, is increased or generated, or if the activity transport protein is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 27 to 129-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a transport protein, or if the activity of the polypeptide B2063, preferably represented by SEQ ID NO. 38948, or a homolog or fragment thereof, or if the activity of a polypeptide encoded by a nucleic acid molecule comprising the nucleic acid SEQ ID NO. 38947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 38947 or polypeptide SEQ ID NO. 38948, respectively, or a homolog or a fragment thereof, is increased or generated, or if the activity transport protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 36 to 750-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a uridine/cytidine kinase, or if the activity of the polypeptide B2066, preferably represented by SEQ ID NO. 8938, or a homolog or fragment thereof, or if the activity of a polypeptide encoded by a nucleic acid molecule comprising the nucleic acid SEQ ID NO. 8937, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 8937 or polypeptide SEQ ID NO. 8938, respectively, or a homolog or a fragment thereof, is increased or generated, or if the activity uridine/cytidine kinase is increased or generated in a non-human organism, like a microorganism or a plant cell, plant or part thereof, especially with mitochondrial localization, whereby the respective line discloses in column 7 5-oxoproline. For example, an increase of the 5-oxoproline of at least 1 percent, particularly in a range of 34 to 113-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a b2107-protein, or if the activity of the polypeptide B2107, preferably represented by SEQ ID NO. 39003, or a homolog or fragment thereof, or if the activity of a polypeptide encoded by a nucleic acid molecule comprising the nucleic acid SEQ ID NO. 39002, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 39002 or polypeptide SEQ ID NO. 39003, respectively, or a homolog or a fragment thereof, is increased or generated, or if the activity b2107-protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 64 to 278-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a ABC transporter permease protein, or if the activity of the polypeptide B2178, preferably represented by SEQ ID NO. 39041, or a homolog or fragment thereof, or if the activity of a polypeptide encoded by a nucleic acid molecule comprising the nucleic acid SEQ ID NO. 39040, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 39040 or polypeptide SEQ ID NO. 39041, respectively, or a homolog or a fragment thereof, is increased or generated, or if the activity ABC transporter permease protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 58 to 132-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a ABC transporter permease protein, or if the activity of the polypeptide B2178, preferably represented by SEQ ID NO. 39041, or a homolog or fragment thereof, or if the activity of a polypeptide encoded by a nucleic acid molecule comprising the nucleic acid SEQ ID NO. 39040, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 39040 or polypeptide SEQ ID NO. 39041, respectively, or a homolog or a fragment thereof, is increased or generated, or if the activity ABC transporter permease protein is increased or generated in a non-human organism, like a microorganism or a plant cell, plant or part thereof, especially with non-targeted localization, whereby the respective line discloses in column 7 citrulline. For example, an increase of the citrulline of at least 1 percent, particularly in a range of 43 to 94-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a ABC transporter permease protein, or if the activity of the polypeptide B2178, preferably represented by SEQ ID NO. 39041, or a homolog or fragment thereof, or if the activity of a polypeptide encoded by a nucleic acid molecule comprising the nucleic acid SEQ ID NO. 39040, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 39040 or polypeptide SEQ ID NO. 39041, respectively, or a homolog or a fragment thereof, is increased or generated, or if the activity ABC transporter permease protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 25 to 44-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a ABC transporter permease protein, or if the activity of the polypeptide B2178, preferably represented by SEQ ID NO. 39041, or a homolog or fragment thereof, or if the activity of a polypeptide encoded by a nucleic acid molecule comprising the nucleic acid SEQ ID NO. 39040, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 39040 or polypeptide SEQ ID NO. 39041, respectively, or a homolog or a fragment thereof, is increased or generated, or if the activity ABC transporter permease protein is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 26 to 64-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a ABC transporter permease protein, or if the activity of the polypeptide B2178, preferably represented by SEQ ID NO. 39041, or a homolog or fragment thereof, or if the activity of a polypeptide encoded by a nucleic acid molecule comprising the nucleic acid SEQ ID NO. 39040, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 39040 or polypeptide SEQ ID NO. 39041, respectively, or a homolog or a fragment thereof, is increased or generated, or if the activity ABC transporter permease protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 38 to 39-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a sn-glycerol-3-phosphate dehydrogenase, or if the activity of the polypeptide B2242, preferably represented by SEQ ID NO. 79280, or a homolog or fragment thereof, or if the activity of a polypeptide encoded by a nucleic acid molecule comprising the nucleic acid SEQ ID NO. 79279, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 79279 or polypeptide SEQ ID NO. 79280, respectively, or a homolog or a fragment thereof, is increased or generated, or if the activity sn-glycerol-3-phosphate dehydrogenase is increased or generated in a non-human organism, like a microorganism or a plant cell, plant or part thereof, especially with plastidic localization, whereby the respective line discloses in column 7 glycine. For example, an increase of the glycine of at least 1 percent, particularly in a range of 30 to 52-percent is conferred as compared to a corresponding non-transformed wild type non-human organism.

Accordingly, in one embodiment, an increase of asparagine in a non-human organism, as compared to a corresponding non-transformed wild type non-human organism, is conferred in the process of the invention, if the activity of a polypeptide showing the activity of a NADH dehydrogenase I chain I, or if the activity of the polypeptide B2281, preferably represented by SEQ ID NO. 39121, or a homolog or fragment thereof, or if the activity of a polypeptide encoded by a nucleic acid molecule comprising the nucleic acid SEQ ID NO. 39120, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 39120 or polypeptide SEQ ID NO. 39121, respectively, or a homolog or a fragment thereof, is increased or generated, or if the activity NADH dehydrogenase I chain I is increased or generated in a non-human organism, like a microorganism or a plant cell, plant or part thereof, especially with non-targeted localization, whereby the respective line discloses in column 7 asparagine. For example, an increase of the asparagine of at least 1 percent, particularly in a range of 34 to 63-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a b2360-protein, or if the activity of the polypeptide B2360, preferably represented by SEQ ID NO. 39220, or a homolog or fragment thereof, or if the activity of a polypeptide encoded by a nucleic acid molecule comprising the nucleic acid SEQ ID NO. 39219, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 39219 or polypeptide SEQ ID NO. 39220, respectively, or a homolog or a fragment thereof, is increased or generated, or if the activity b2360-protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 68 to 124-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a D-serine dehydratase, or if the activity of the polypeptide B2366, preferably represented by SEQ ID NO. 79343, or a homolog or fragment thereof, or if the activity of a polypeptide encoded by a nucleic acid molecule comprising the nucleic acid SEQ ID NO. 79342, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 79342 or polypeptide SEQ ID NO. 79343, respectively, or a homolog or a fragment thereof, is increased or generated, or if the activity D-serine dehydratase is increased or generated in a non-human organism, like a microorganism or a plant cell, plant or part thereof, especially with plastidic localization, whereby the respective line discloses in column 7 serine. For example, an increase of the serine of at least 1 percent, particularly in a range of 24 to 67-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a b2399-protein, or if the activity of the polypeptide B2399, preferably represented by SEQ ID NO. 39238, or a homolog or fragment thereof, or if the activity of a polypeptide encoded by a nucleic acid molecule comprising the nucleic acid SEQ ID NO. 39237, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 39237 or polypeptide SEQ ID NO. 39238, respectively, or a homolog or a fragment thereof, is increased or generated, or if the activity b2399-protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 37 to 156-percent is conferred as compared to a corresponding non-transformed wild type non-human organism.

Accordingly, in one embodiment, an increase of 3,4-dihydroxyphenylalanine (dopa) in a non-human organism, as compared to a corresponding non-transformed wild type non-human organism, is conferred in the process of the invention, if the activity of a polypeptide showing the activity of a b2399-protein, or if the activity of the polypeptide B2399, preferably represented by SEQ ID NO. 39238, or a homolog or fragment thereof, or if the activity of a polypeptide encoded by a nucleic acid molecule comprising the nucleic acid SEQ ID NO. 39237, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 39237 or polypeptide SEQ ID NO. 39238, respectively, or a homolog or a fragment thereof, is increased or generated, or if the activity b2399-protein is increased or generated in a non-human organism, like a microorganism or a plant cell, plant or part thereof, especially with non-targeted localization, whereby the respective line discloses in column 7 3,4-dihydroxyphenylalanine (dopa). For example, an increase of the 3,4-dihydroxyphenylalanine (dopa) of at least 1 percent, particularly in a range of 44 to 168-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a b2399-protein, or if the activity of the polypeptide B2399, preferably represented by SEQ ID NO. 39238, or a homolog or fragment thereof, or if the activity of a polypeptide encoded by a nucleic acid molecule comprising the nucleic acid SEQ ID NO. 39237, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 39237 or polypeptide SEQ ID NO. 39238, respectively, or a homolog or a fragment thereof, is increased or generated, or if the activity b2399-protein is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 48 to 144-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a transcriptional regulator, or if the activity of the polypeptide B2405, preferably represented by SEQ ID NO. 39256, or a homolog or fragment thereof, or if the activity of a polypeptide encoded by a nucleic acid molecule comprising the nucleic acid SEQ ID NO. 39255, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 39255 or polypeptide SEQ ID NO. 39256, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 28 to 51-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a transcriptional regulator, or if the activity of the polypeptide B2405, preferably represented by SEQ ID NO. 39256, or a homolog or fragment thereof, or if the activity of a polypeptide encoded by a nucleic acid molecule comprising the nucleic acid SEQ ID NO. 39255, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 39255 or polypeptide SEQ ID NO. 39256, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 52 to 87-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a PTS system N-acetylmuramic acid-specific EIIBC component, or if the activity of the polypeptide B2429, preferably represented by SEQ ID NO. 79399, or a homolog or fragment thereof, or if the activity of a polypeptide encoded by a nucleic acid molecule comprising the nucleic acid SEQ ID NO. 79398, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 79398 or polypeptide SEQ ID NO. 79399, respectively, or a homolog or a fragment thereof, is increased or generated, or if the activity PTS system N-acetylmuramic acid-specific EIIBC component is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 37 to 52-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a b2474-protein, or if the activity of the polypeptide B2474, preferably represented by SEQ ID NO. 40330, or a homolog or fragment thereof, or if the activity of a polypeptide encoded by a nucleic acid molecule comprising the nucleic acid SEQ ID NO. 40329, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 40329 or polypeptide SEQ ID NO. 40330, respectively, or a homolog or a fragment thereof, is increased or generated, or if the activity b2474-protein is increased or generated in a non-human organism, like a microorganism or a plant cell, plant or part thereof, especially with non-targeted localization, whereby the respective line discloses in column 7 citrulline. For example, an increase of the citrulline of at least 1 percent, particularly in a range of 31 to 119-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a b2474-protein, or if the activity of the polypeptide B2474, preferably represented by SEQ ID NO. 40330, or a homolog or fragment thereof, or if the activity of a polypeptide encoded by a nucleic acid molecule comprising the nucleic acid SEQ ID NO. 40329, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 40329 or polypeptide SEQ ID NO. 40330, respectively, or a homolog or a fragment thereof, is increased or generated, or if the activity b2474-protein is increased or generated in a non-human organism, like a microorganism or a plant cell, plant or part thereof, especially with non-targeted localization, whereby the respective line discloses in column 7 glycine. For example, an increase of the glycine of at least 1 percent, particularly in a range of 41 to 71-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a NADH dehydrogenase subunit N, or if the activity of the polypeptide B2482, preferably represented by SEQ ID NO. 79510, or a homolog or fragment thereof, or if the activity of a polypeptide encoded by a nucleic acid molecule comprising the nucleic acid SEQ ID NO. 79509, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 79509 or polypeptide SEQ ID NO. 79510, respectively, or a homolog or a fragment thereof, is increased or generated, or if the activity NADH dehydrogenase subunit N is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 24 to 62-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a NADH dehydrogenase subunit N, or if the activity of the polypeptide B2482, preferably represented by SEQ ID NO. 79510, or a homolog or fragment thereof, or if the activity of a polypeptide encoded by a nucleic acid molecule comprising the nucleic acid SEQ ID NO. 79509, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 79509 or polypeptide SEQ ID NO. 79510, respectively, or a homolog or a fragment thereof, is increased or generated, or if the activity NADH dehydrogenase subunit N is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 40 to 97-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a b2513-protein, or if the activity of the polypeptide B2513, preferably represented by SEQ ID NO. 9168, or a homolog or fragment thereof, or if the activity of a polypeptide encoded by a nucleic acid molecule comprising the nucleic acid SEQ ID NO. 9167, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 9167 or polypeptide SEQ ID NO. 9168, respectively, or a homolog or a fragment thereof, is increased or generated, or if the activity b2513-protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 65 to 206-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a b2513-protein, or if the activity of the polypeptide B2513, preferably represented by SEQ ID NO. 9168, or a homolog or fragment thereof, or if the activity of a polypeptide encoded by a nucleic acid molecule comprising the nucleic acid SEQ ID NO. 9167, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 9167 or polypeptide SEQ ID NO. 9168, respectively, or a homolog or a fragment thereof, is increased or generated, or if the activity b2513-protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 38 to 456-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a serine hydroxymethyltransferase, or if the activity of the polypeptide B2551, preferably represented by SEQ ID NO. 79628, or a homolog or fragment thereof, or if the activity of a polypeptide encoded by a nucleic acid molecule comprising the nucleic acid SEQ ID NO. 79627, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 79627 or polypeptide SEQ ID NO. 79628, respectively, or a homolog or a fragment thereof, is increased or generated, or if the activity serine hydroxymethyltransferase is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 28 to 120-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a chorismate mutase-T and prephenate dehydrogenase, or if the activity of the polypeptide B2600, preferably represented by SEQ ID NO. 80522, or a homolog or fragment thereof, or if the activity of a polypeptide encoded by a nucleic acid molecule comprising the nucleic acid SEQ ID NO. 80521, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 80521 or polypeptide SEQ ID NO. 80522, respectively, or a homolog or a fragment thereof, is increased or generated, or if the activity chorismate mutase-T and prephenate dehydrogenase is increased or generated in a non-human organism, like a microorganism or a plant cell, plant or part thereof, especially with plastidic localization, whereby the respective line discloses in column 7 phenylalanine. For example, an increase of the phenylalanine of at least 1 percent, particularly in a range of 40 to 334-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a b2613-protein, or if the activity of the polypeptide B2613, preferably represented by SEQ ID NO. 40666, or a homolog or fragment thereof, or if the activity of a polypeptide encoded by a nucleic acid molecule comprising the nucleic acid SEQ ID NO. 40665, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 40665 or polypeptide SEQ ID NO. 40666, respectively, or a homolog or a fragment thereof, is increased or generated, or if the activity b2613-protein is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 27 to 27-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a transcriptional regulator, or if the activity of the polypeptide B2634, preferably represented by SEQ ID NO. 40727, or a homolog or fragment thereof, or if the activity of a polypeptide encoded by a nucleic acid molecule comprising the nucleic acid SEQ ID NO. 40726, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 40726 or polypeptide SEQ ID NO. 40727, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 28 to 421-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a transcriptional regulator, or if the activity of the polypeptide B2634, preferably represented by SEQ ID NO. 40727, or a homolog or fragment thereof, or if the activity of a polypeptide encoded by a nucleic acid molecule comprising the nucleic acid SEQ ID NO. 40726, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 40726 or polypeptide SEQ ID NO. 40727, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 35 to 1033-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a b2673-protein, or if the activity of the polypeptide B2673, preferably represented by SEQ ID NO. 9245, or a homolog or fragment thereof, or if the activity of a polypeptide encoded by a nucleic acid molecule comprising the nucleic acid SEQ ID NO. 9244, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 9244 or polypeptide SEQ ID NO. 9245, respectively, or a homolog or a fragment thereof, is increased or generated, or if the activity b2673-protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 25 to 121-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a b2673-protein, or if the activity of the polypeptide B2673, preferably represented by SEQ ID NO. 9245, or a homolog or fragment thereof, or if the activity of a polypeptide encoded by a nucleic acid molecule comprising the nucleic acid SEQ ID NO. 9244, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 9244 or polypeptide SEQ ID NO. 9245, respectively, or a homolog or a fragment thereof, is increased or generated, or if the activity b2673-protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 46 to 58-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a transport protein, or if the activity of the polypeptide B2680, preferably represented by SEQ ID NO. 80590, or a homolog or fragment thereof, or if the activity of a polypeptide encoded by a nucleic acid molecule comprising the nucleic acid SEQ ID NO. 80589, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 80589 or polypeptide SEQ ID NO. 80590, respectively, or a homolog or a fragment thereof, is increased or generated, or if the activity transport protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 51 to 124-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a murein transglycosylase, or if the activity of the polypeptide B2701, preferably represented by SEQ ID NO. 80757, or a homolog or fragment thereof, or if the activity of a polypeptide encoded by a nucleic acid molecule comprising the nucleic acid SEQ ID NO. 80756, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 80756 or polypeptide SEQ ID NO. 80757, respectively, or a homolog or a fragment thereof, is increased or generated, or if the activity murein transglycosylase is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 19 to 36-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a murein transglycosylase, or if the activity of the polypeptide B2701, preferably represented by SEQ ID NO. 80757, or a homolog or fragment thereof, or if the activity of a polypeptide encoded by a nucleic acid molecule comprising the nucleic acid SEQ ID NO. 80756, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 80756 or polypeptide SEQ ID NO. 80757, respectively, or a homolog or a fragment thereof, is increased or generated, or if the activity murein transglycosylase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 27 to 317-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a murein transglycosylase, or if the activity of the polypeptide B2701, preferably represented by SEQ ID NO. 80757, or a homolog or fragment thereof, or if the activity of a polypeptide encoded by a nucleic acid molecule comprising the nucleic acid SEQ ID NO. 80756, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 80756 or polypeptide SEQ ID NO. 80757, respectively, or a homolog or a fragment thereof, is increased or generated, or if the activity murein transglycosylase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 143 to 237-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a transcriptional regulator, or if the activity of the polypeptide B2714, preferably represented by SEQ ID NO. 40742, or a homolog or fragment thereof, or if the activity of a polypeptide encoded by a nucleic acid molecule comprising the nucleic acid SEQ ID NO. 40741, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 40741 or polypeptide SEQ ID NO. 40742, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 26 to 48-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a b2739-protein, or if the activity of the polypeptide B2739, preferably represented by SEQ ID NO. 80907, or a homolog or fragment thereof, or if the activity of a polypeptide encoded by a nucleic acid molecule comprising the nucleic acid SEQ ID NO. 80906, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 80906 or polypeptide SEQ ID NO. 80907, respectively, or a homolog or a fragment thereof, is increased or generated, or if the activity b2739-protein is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 21 to 76-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a b2739-protein, or if the activity of the polypeptide B2739, preferably represented by SEQ ID NO. 80907, or a homolog or fragment thereof, or if the activity of a polypeptide encoded by a nucleic acid molecule comprising the nucleic acid SEQ ID NO. 80906, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 80906 or polypeptide SEQ ID NO. 80907, respectively, or a homolog or a fragment thereof, is increased or generated, or if the activity b2739-protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 25 to 234-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a b2739-protein, or if the activity of the polypeptide B2739, preferably represented by SEQ ID NO. 80907, or a homolog or fragment thereof, or if the activity of a polypeptide encoded by a nucleic acid molecule comprising the nucleic acid SEQ ID NO. 80906, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 80906 or polypeptide SEQ ID NO. 80907, respectively, or a homolog or a fragment thereof, is increased or generated, or if the activity b2739-protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 40 to 595-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a 2-C-methyl-D-erythritol 4-phosphate cytidylyltransferase, or if the activity of the polypeptide B2747, preferably represented by SEQ ID NO. 81076, or a homolog or fragment thereof, or if the activity of a polypeptide encoded by a nucleic acid molecule comprising the nucleic acid SEQ ID NO. 81075, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 81075 or polypeptide SEQ ID NO. 81076, respectively, or a homolog or a fragment thereof, is increased or generated, or if the activity 2-C-methyl-D-erythritol 4-phosphate cytidylyltransferase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 68 to 92-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a 2-C-methyl-D-erythritol 4-phosphate cytidylyltransferase, or if the activity of the polypeptide B2747, preferably represented by SEQ ID NO. 81076, or a homolog or fragment thereof, or if the activity of a polypeptide encoded by a nucleic acid molecule comprising the nucleic acid SEQ ID NO. 81075, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 81075 or polypeptide SEQ ID NO. 81076, respectively, or a homolog or a fragment thereof, is increased or generated, or if the activity 2-C-methyl-D-erythritol 4-phosphate cytidylyltransferase is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 43 to 297-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a phosphoadenosine phosphosulfate reductase, or if the activity of the polypeptide B2762, preferably represented by SEQ ID NO. 81452, or a homolog or fragment thereof, or if the activity of a polypeptide encoded by a nucleic acid molecule comprising the nucleic acid SEQ ID NO. 81451, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 81451 or polypeptide SEQ ID NO. 81452, respectively, or a homolog or a fragment thereof, is increased or generated, or if the activity phosphoadenosine phosphosulfate reductase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 25 to 78-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a b2812-protein, or if the activity of the polypeptide B2812, preferably represented by SEQ ID NO. 40796, or a homolog or fragment thereof, or if the activity of a polypeptide encoded by a nucleic acid molecule comprising the nucleic acid SEQ ID NO. 40795, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 40795 or polypeptide SEQ ID NO. 40796, respectively, or a homolog or a fragment thereof, is increased or generated, or if the activity b2812-protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 44 to 99-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a b2812-protein, or if the activity of the polypeptide B2812, preferably represented by SEQ ID NO. 40796, or a homolog or fragment thereof, or if the activity of a polypeptide encoded by a nucleic acid molecule comprising the nucleic acid SEQ ID NO. 40795, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 40795 or polypeptide SEQ ID NO. 40796, respectively, or a homolog or a fragment thereof, is increased or generated, or if the activity b2812-protein is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 26 to 123-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a b2812-protein, or if the activity of the polypeptide B2812, preferably represented by SEQ ID NO. 40796, or a homolog or fragment thereof, or if the activity of a polypeptide encoded by a nucleic acid molecule comprising the nucleic acid SEQ ID NO. 40795, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 40795 or polypeptide SEQ ID NO. 40796, respectively, or a homolog or a fragment thereof, is increased or generated, or if the activity b2812-protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 38 to 122-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a amino-acid acetyltransferase, or if the activity of the polypeptide B2818, preferably represented by SEQ ID NO. 81577, or a homolog or fragment thereof, or if the activity of a polypeptide encoded by a nucleic acid molecule comprising the nucleic acid SEQ ID NO. 81576, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 81576 or polypeptide SEQ ID NO. 81577, respectively, or a homolog or a fragment thereof, is increased or generated, or if the activity amino-acid acetyltransferase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 27 to 826-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a amino-acid acetyltransferase, or if the activity of the polypeptide B2818, preferably represented by SEQ ID NO. 81577, or a homolog or fragment thereof, or if the activity of a polypeptide encoded by a nucleic acid molecule comprising the nucleic acid SEQ ID NO. 81576, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 81576 or polypeptide SEQ ID NO. 81577, respectively, or a homolog or a fragment thereof, is increased or generated, or if the activity amino-acid acetyltransferase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 41 to 427-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a amino-acid acetyltransferase, or if the activity of the polypeptide B2818, preferably represented by SEQ ID NO. 81577, or a homolog or fragment thereof, or if the activity of a polypeptide encoded by a nucleic acid molecule comprising the nucleic acid SEQ ID NO. 81576, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 81576 or polypeptide SEQ ID NO. 81577, respectively, or a homolog or a fragment thereof, is increased or generated, or if the activity amino-acid acetyltransferase is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 34 to 266-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a prolipoprotein diacylglyceryl transferase, or if the activity of the polypeptide B2828, preferably represented by SEQ ID NO. 81693, or a homolog or fragment thereof, or if the activity of a polypeptide encoded by a nucleic acid molecule comprising the nucleic acid SEQ ID NO. 81692, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 81692 or polypeptide SEQ ID NO. 81693, respectively, or a homolog or a fragment thereof, is increased or generated, or if the activity prolipoprotein diacylglyceryl transferase is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 40 to 154-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a transcriptional regulator, or if the activity of the polypeptide B2839, preferably represented by SEQ ID NO. 81936, or a homolog or fragment thereof, or if the activity of a polypeptide encoded by a nucleic acid molecule comprising the nucleic acid SEQ ID NO. 81935, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 81935 or polypeptide SEQ ID NO. 81936, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 42 to 55-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a b2849-protein, or if the activity of the polypeptide B2849, preferably represented by SEQ ID NO. 81981, or a homolog or fragment thereof, or if the activity of a polypeptide encoded by a nucleic acid molecule comprising the nucleic acid SEQ ID NO. 81980, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 81980 or polypeptide SEQ ID NO. 81981, respectively, or a homolog or a fragment thereof, is increased or generated, or if the activity b2849-protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 26 to 78-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a D-3-phosphoglycerate dehydrogenase, or if the activity of the polypeptide B2913, preferably represented by SEQ ID NO. 81989, or a homolog or fragment thereof, or if the activity of a polypeptide encoded by a nucleic acid molecule comprising the nucleic acid SEQ ID NO. 81988, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 81988 or polypeptide SEQ ID NO. 81989, respectively, or a homolog or a fragment thereof, is increased or generated, or if the activity D-3-phosphoglycerate dehydrogenase is increased or generated in a non-human organism, like a microorganism or a plant cell, plant or part thereof, especially with plastidic localization, whereby the respective line discloses in column 7 glycine. For example, an increase of the glycine of at least 1 percent, particularly in a range of 31 to 168-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a D-3-phosphoglycerate dehydrogenase, or if the activity of the polypeptide B2913, preferably represented by SEQ ID NO. 81989, or a homolog or fragment thereof, or if the activity of a polypeptide encoded by a nucleic acid molecule comprising the nucleic acid SEQ ID NO. 81988, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 81988 or polypeptide SEQ ID NO. 81989, respectively, or a homolog or a fragment thereof, is increased or generated, or if the activity D-3-phosphoglycerate dehydrogenase is increased or generated in a non-human organism, like a microorganism or a plant cell, plant or part thereof, especially with plastidic localization, whereby the respective line discloses in column 7 serine. For example, an increase of the serine of at least 1 percent, particularly in a range of 28 to 215-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a arginine exporter protein, or if the activity of the polypeptide B2923, preferably represented by SEQ ID NO. 9334, or a homolog or fragment thereof, or if the activity of a polypeptide encoded by a nucleic acid molecule comprising the nucleic acid SEQ ID NO. 9333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 9333 or polypeptide SEQ ID NO. 9334, respectively, or a homolog or a fragment thereof, is increased or generated, or if the activity arginine exporter protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 64 to 126-percent is conferred as compared to a corresponding non-transformed wild type non-human organism.

Accordingly, in one embodiment, an increase of asparagine in a non-human organism, as compared to a corresponding non-transformed wild type non-human organism, is conferred in the process of the invention, if the activity of a polypeptide showing the activity of a glycoprotease, or if the activity of the polypeptide B3064, preferably represented by SEQ ID NO. 42047, or a homolog or fragment thereof, or if the activity of a polypeptide encoded by a nucleic acid molecule comprising the nucleic acid SEQ ID NO. 42046, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 42046 or polypeptide SEQ ID NO. 42047, respectively, or a homolog or a fragment thereof, is increased or generated, or if the activity glycoprotease is increased or generated in a non-human organism, like a microorganism or a plant cell, plant or part thereof, especially with non-targeted localization, whereby the respective line discloses in column 7 asparagine. For example, an increase of the asparagine of at least 1 percent, particularly in a range of 30 to 189-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a b3083-protein, or if the activity of the polypeptide B3083, preferably represented by SEQ ID NO. 82425, or a homolog or fragment thereof, or if the activity of a polypeptide encoded by a nucleic acid molecule comprising the nucleic acid SEQ ID NO. 82424, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 82424 or polypeptide SEQ ID NO. 82425, respectively, or a homolog or a fragment thereof, is increased or generated, or if the activity b3083-protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 28 to 61-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a b3083-protein, or if the activity of the polypeptide B3083, preferably represented by SEQ ID NO. 82425, or a homolog or fragment thereof, or if the activity of a polypeptide encoded by a nucleic acid molecule comprising the nucleic acid SEQ ID NO. 82424, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 82424 or polypeptide SEQ ID NO. 82425, respectively, or a homolog or a fragment thereof, is increased or generated, or if the activity b3083-protein is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 23 to 108-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a b3083-protein, or if the activity of the polypeptide B3083, preferably represented by SEQ ID NO. 82425, or a homolog or fragment thereof, or if the activity of a polypeptide encoded by a nucleic acid molecule comprising the nucleic acid SEQ ID NO. 82424, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 82424 or polypeptide SEQ ID NO. 82425, respectively, or a homolog or a fragment thereof, is increased or generated, or if the activity b3083-protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 35 to 72-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a b3098-protein, or if the activity of the polypeptide B3098, preferably represented by SEQ ID NO. 82460, or a homolog or fragment thereof, or if the activity of a polypeptide encoded by a nucleic acid molecule comprising the nucleic acid SEQ ID NO. 82459, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 82459 or polypeptide SEQ ID NO. 82460, respectively, or a homolog or a fragment thereof, is increased or generated, or if the activity b3098-protein is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 31 to 58-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a b3098-protein, or if the activity of the polypeptide B3098, preferably represented by SEQ ID NO. 82460, or a homolog or fragment thereof, or if the activity of a polypeptide encoded by a nucleic acid molecule comprising the nucleic acid SEQ ID NO. 82459, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 82459 or polypeptide SEQ ID NO. 82460, respectively, or a homolog or a fragment thereof, is increased or generated, or if the activity b3098-protein is increased or generated in a non-human organism, like a microorganism or a plant cell, plant or part thereof, especially with non-targeted localization, whereby the respective line discloses in column 7 glycine. For example, an increase of the glycine of at least 1 percent, particularly in a range of 38 to 76-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a b3121-protein, or if the activity of the polypeptide B3121, preferably represented by SEQ ID NO. 42472, or a homolog or fragment thereof, or if the activity of a polypeptide encoded by a nucleic acid molecule comprising the nucleic acid SEQ ID NO. 42471, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 42471 or polypeptide SEQ ID NO. 42472, respectively, or a homolog or a fragment thereof, is increased or generated, or if the activity b3121-protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 39 to 73-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a b3221-protein, or if the activity of the polypeptide B3221, preferably represented by SEQ ID NO. 82511, or a homolog or fragment thereof, or if the activity of a polypeptide encoded by a nucleic acid molecule comprising the nucleic acid SEQ ID NO. 82510, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 82510 or polypeptide SEQ ID NO. 82511, respectively, or a homolog or a fragment thereof, is increased or generated, or if the activity b3221-protein is increased or generated in a non-human organism, like a microorganism or a plant cell, plant or part thereof, especially with plastidic localization, whereby the respective line discloses in column 7 tyrosine. For example, an increase of the tyrosine of at least 1 percent, particularly in a range of 37 to 55-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a b3246-protein, or if the activity of the polypeptide B3246, preferably represented by SEQ ID NO. 9471, or a homolog or fragment thereof, or if the activity of a polypeptide encoded by a nucleic acid molecule comprising the nucleic acid SEQ ID NO. 9470, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 9470 or polypeptide SEQ ID NO. 9471, respectively, or a homolog or a fragment thereof, is increased or generated, or if the activity b3246-protein is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 24 to 101-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a b3246-protein, or if the activity of the polypeptide B3246, preferably represented by SEQ ID NO. 9471, or a homolog or fragment thereof, or if the activity of a polypeptide encoded by a nucleic acid molecule comprising the nucleic acid SEQ ID NO. 9470, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 9470 or polypeptide SEQ ID NO. 9471, respectively, or a homolog or a fragment thereof, is increased or generated, or if the activity b3246-protein is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 33 to 89-percent is conferred as compared to a corresponding non-transformed wild type non-human organism.

Accordingly, in one embodiment, an increase of asparagine in a non-human organism, as compared to a corresponding non-transformed wild type non-human organism, is conferred in the process of the invention, if the activity of a polypeptide showing the activity of a acetyl CoA carboxylase, or if the activity of the polypeptide B3256, preferably represented by SEQ ID NO. 9493, or a homolog or fragment thereof, or if the activity of a polypeptide encoded by a nucleic acid molecule comprising the nucleic acid SEQ ID NO. 9492, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 9492 or polypeptide SEQ ID NO. 9493, respectively, or a homolog or a fragment thereof, is increased or generated, or if the activity acetyl CoA carboxylase is increased or generated in a non-human organism, like a microorganism or a plant cell, plant or part thereof, especially with non-targeted localization, whereby the respective line discloses in column 7 asparagine. For example, an increase of the asparagine of at least 1 percent, particularly in a range of 67 to 286-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a acetyl CoA carboxylase, or if the activity of the polypeptide B3256, preferably represented by SEQ ID NO. 9493, or a homolog or fragment thereof, or if the activity of a polypeptide encoded by a nucleic acid molecule comprising the nucleic acid SEQ ID NO. 9492, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 9492 or polypeptide SEQ ID NO. 9493, respectively, or a homolog or a fragment thereof, is increased or generated, or if the activity acetyl CoA carboxylase is increased or generated in a non-human organism, like a microorganism or a plant cell, plant or part thereof, especially with non-targeted localization, whereby the respective line discloses in column 7 glycine. For example, an increase of the glycine of at least 1 percent, particularly in a range of 28 to 344-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a acetyl CoA carboxylase, or if the activity of the polypeptide B3256, preferably represented by SEQ ID NO. 9493, or a homolog or fragment thereof, or if the activity of a polypeptide encoded by a nucleic acid molecule comprising the nucleic acid SEQ ID NO. 9492, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 9492 or polypeptide SEQ ID NO. 9493, respectively, or a homolog or a fragment thereof, is increased or generated, or if the activity acetyl CoA carboxylase is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 31 to 1323-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a acetyl CoA carboxylase, or if the activity of the polypeptide B3256, preferably represented by SEQ ID NO. 9493, or a homolog or fragment thereof, or if the activity of a polypeptide encoded by a nucleic acid molecule comprising the nucleic acid SEQ ID NO. 9492, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 9492 or polypeptide SEQ ID NO. 9493, respectively, or a homolog or a fragment thereof, is increased or generated, or if the activity acetyl CoA carboxylase is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 32 to 175-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a methyltransferase, or if the activity of the polypeptide B3262, preferably represented by SEQ ID NO. 42503, or a homolog or fragment thereof, or if the activity of a polypeptide encoded by a nucleic acid molecule comprising the nucleic acid SEQ ID NO. 42502, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 42502 or polypeptide SEQ ID NO. 42503, respectively, or a homolog or a fragment thereof, is increased or generated, or if the activity methyltransferase is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 25 to 58-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a methyltransferase, or if the activity of the polypeptide B3262, preferably represented by SEQ ID NO. 42503, or a homolog or fragment thereof, or if the activity of a polypeptide encoded by a nucleic acid molecule comprising the nucleic acid SEQ ID NO. 42502, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 42502 or polypeptide SEQ ID NO. 42503, respectively, or a homolog or a fragment thereof, is increased or generated, or if the activity methyltransferase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 36 to 199-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a methyltransferase, or if the activity of the polypeptide B3262, preferably represented by SEQ ID NO. 42503, or a homolog or fragment thereof, or if the activity of a polypeptide encoded by a nucleic acid molecule comprising the nucleic acid SEQ ID NO. 42502, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 42502 or polypeptide SEQ ID NO. 42503, respectively, or a homolog or a fragment thereof, is increased or generated, or if the activity methyltransferase is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 34 to 141-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a methyltransferase, or if the activity of the polypeptide B3262, preferably represented by SEQ ID NO. 42503, or a homolog or fragment thereof, or if the activity of a polypeptide encoded by a nucleic acid molecule comprising the nucleic acid SEQ ID NO. 42502, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 42502 or polypeptide SEQ ID NO. 42503, respectively, or a homolog or a fragment thereof, is increased or generated, or if the activity methyltransferase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 45 to 322-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a methyltransferase, or if the activity of the polypeptide B3262, preferably represented by SEQ ID NO. 42503, or a homolog or fragment thereof, or if the activity of a polypeptide encoded by a nucleic acid molecule comprising the nucleic acid SEQ ID NO. 42502, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 42502 or polypeptide SEQ ID NO. 42503, respectively, or a homolog or a fragment thereof, is increased or generated, or if the activity methyltransferase is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 72 to 179-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a b3346-protein, or if the activity of the polypeptide B3346, preferably represented by SEQ ID NO. 10105, or a homolog or fragment thereof, or if the activity of a polypeptide encoded by a nucleic acid molecule comprising the nucleic acid SEQ ID NO. 10104, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 10104 or polypeptide SEQ ID NO. 10105, respectively, or a homolog or a fragment thereof, is increased or generated, or if the activity b3346-protein is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 20 to 53-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a ABC transporter ATP-binding protein, or if the activity of the polypeptide B3352, preferably represented by SEQ ID NO. 82573, or a homolog or fragment thereof, or if the activity of a polypeptide encoded by a nucleic acid molecule comprising the nucleic acid SEQ ID NO. 82572, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 82572 or polypeptide SEQ ID NO. 82573, respectively, or a homolog or a fragment thereof, is increased or generated, or if the activity ABC transporter ATP-binding protein is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 19 to 45-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a ABC transporter ATP-binding protein, or if the activity of the polypeptide B3352, preferably represented by SEQ ID NO. 82573, or a homolog or fragment thereof, or if the activity of a polypeptide encoded by a nucleic acid molecule comprising the nucleic acid SEQ ID NO. 82572, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 82572 or polypeptide SEQ ID NO. 82573, respectively, or a homolog or a fragment thereof, is increased or generated, or if the activity ABC transporter ATP-binding protein is increased or generated in a non-human organism, like a microorganism or a plant cell, plant or part thereof, especially with non-targeted localization, whereby the respective line discloses in column 7 glycine. For example, an increase of the glycine of at least 1 percent, particularly in a range of 40 to 80-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a b3392-protein, or if the activity of the polypeptide B3392, preferably represented by SEQ ID NO. 82847, or a homolog or fragment thereof, or if the activity of a polypeptide encoded by a nucleic acid molecule comprising the nucleic acid SEQ ID NO. 82846, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 82846 or polypeptide SEQ ID NO. 82847, respectively, or a homolog or a fragment thereof, is increased or generated, or if the activity b3392-protein is increased or generated in a non-human organism, like a microorganism or a plant cell, plant or part thereof, especially with plastidic localization, whereby the respective line discloses in column 7 aspartate. For example, an increase of the aspartate of at least 1 percent, particularly in a range of 41 to 49-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a phosphoenolpyruvate carboxykinase, or if the activity of the polypeptide B3403, preferably represented by SEQ ID NO. 82867, or a homolog or fragment thereof, or if the activity of a polypeptide encoded by a nucleic acid molecule comprising the nucleic acid SEQ ID NO. 82866, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 82866 or polypeptide SEQ ID NO. 82867, respectively, or a homolog or a fragment thereof, is increased or generated, or if the activity phosphoenolpyruvate carboxykinase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 26 to 63-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a phosphoenolpyruvate carboxykinase, or if the activity of the polypeptide B3403, preferably represented by SEQ ID NO. 82867, or a homolog or fragment thereof, or if the activity of a polypeptide encoded by a nucleic acid molecule comprising the nucleic acid SEQ ID NO. 82866, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 82866 or polypeptide SEQ ID NO. 82867, respectively, or a homolog or a fragment thereof, is increased or generated, or if the activity phosphoenolpyruvate carboxykinase is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 31 to 45-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a b3410-protein, or if the activity of the polypeptide B3410, preferably represented by SEQ ID NO. 42560, or a homolog or fragment thereof, or if the activity of a polypeptide encoded by a nucleic acid molecule comprising the nucleic acid SEQ ID NO. 42559, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 42559 or polypeptide SEQ ID NO. 42560, respectively, or a homolog or a fragment thereof, is increased or generated, or if the activity b3410-protein is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 23 to 65-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a b3509-protein, or if the activity of the polypeptide B3509, preferably represented by SEQ ID NO. 42593, or a homolog or fragment thereof, or if the activity of a polypeptide encoded by a nucleic acid molecule comprising the nucleic acid SEQ ID NO. 42592, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 42592 or polypeptide SEQ ID NO. 42593, respectively, or a homolog or a fragment thereof, is increased or generated, or if the activity b3509-protein is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 26 to 63-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a cellulose synthase catalytic subunit, or if the activity of the polypeptide B3533, preferably represented by SEQ ID NO. 83154, or a homolog or fragment thereof, or if the activity of a polypeptide encoded by a nucleic acid molecule comprising the nucleic acid SEQ ID NO. 83153, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 83153 or polypeptide SEQ ID NO. 83154, respectively, or a homolog or a fragment thereof, is increased or generated, or if the activity cellulose synthase catalytic subunit is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 37 to 669-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a serine acetyltransferase, or if the activity of the polypeptide B3607, preferably represented by SEQ ID NO. 83214, or a homolog or fragment thereof, or if the activity of a polypeptide encoded by a nucleic acid molecule comprising the nucleic acid SEQ ID NO. 83213, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 83213 or polypeptide SEQ ID NO. 83214, respectively, or a homolog or a fragment thereof, is increased or generated, or if the activity serine acetyltransferase is increased or generated in a non-human organism, like a microorganism or a plant cell, plant or part thereof, especially with plastidic localization, whereby the respective line discloses in column 7 phenylalanine. For example, an increase of the phenylalanine of at least 1 percent, particularly in a range of 46 to 60-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a serine acetyltransferase, or if the activity of the polypeptide B3607, preferably represented by SEQ ID NO. 83214, or a homolog or fragment thereof, or if the activity of a polypeptide encoded by a nucleic acid molecule comprising the nucleic acid SEQ ID NO. 83213, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 83213 or polypeptide SEQ ID NO. 83214, respectively, or a homolog or a fragment thereof, is increased or generated, or if the activity serine acetyltransferase is increased or generated in a non-human organism, like a microorganism or a plant cell, plant or part thereof, especially with plastidic localization, whereby the respective line discloses in column 7 tyrosine. For example, an increase of the tyrosine of at least 1 percent, particularly in a range of 29 to 82-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a phosphopantetheine adenylyltransferase, or if the activity of the polypeptide B3634, preferably represented by SEQ ID NO. 42932, or a homolog or fragment thereof, or if the activity of a polypeptide encoded by a nucleic acid molecule comprising the nucleic acid SEQ ID NO. 42931, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 42931 or polypeptide SEQ ID NO. 42932, respectively, or a homolog or a fragment thereof, is increased or generated, or if the activity phosphopantetheine adenylyltransferase is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 21 to 39 percent is conferred as compared to a corresponding non-transformed wild type non-human organism.

Accordingly, in one embodiment, an increase of asparagine in a non-human organism, as compared to a corresponding non-transformed wild type non-human organism, is conferred in the process of the invention, if the activity of a polypeptide showing the activity of a phosphopantetheine adenylyltransferase, or if the activity of the polypeptide B3634, preferably represented by SEQ ID NO. 42932, or a homolog or fragment thereof, or if the activity of a polypeptide encoded by a nucleic acid molecule comprising the nucleic acid SEQ ID NO. 42931, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 42931 or polypeptide SEQ ID NO. 42932, respectively, or a homolog or a fragment thereof, is increased or generated, or if the activity phosphopantetheine adenylyltransferase is increased or generated in a non-human organism, like a microorganism or a plant cell, plant or part thereof, especially with non-targeted localization, whereby the respective line discloses in column 7 asparagine. For example, an increase of the asparagine of at least 1 percent, particularly in a range of 44 to 169-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a phosphopantetheine adenylyltransferase, or if the activity of the polypeptide B3634, preferably represented by SEQ ID NO. 42932, or a homolog or fragment thereof, or if the activity of a polypeptide encoded by a nucleic acid molecule comprising the nucleic acid SEQ ID NO. 42931, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 42931 or polypeptide SEQ ID NO. 42932, respectively, or a homolog or a fragment thereof, is increased or generated, or if the activity phosphopantetheine adenylyltransferase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 82 to 92-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a b3646-protein, or if the activity of the polypeptide B3646, preferably represented by SEQ ID NO. 83430, or a homolog or fragment thereof, or if the activity of a polypeptide encoded by a nucleic acid molecule comprising the nucleic acid SEQ ID NO. 83429, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 83429 or polypeptide SEQ ID NO. 83430, respectively, or a homolog or a fragment thereof, is increased or generated, or if the activity b3646-protein is increased or generated in a non-human organism, like a microorganism or a plant cell, plant or part thereof, especially with non-targeted localization, whereby the respective line discloses in column 7 glycine. For example, an increase of the glycine of at least 1 percent, particularly in a range of 35 to 1154-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a acetolactate synthase, or if the activity of the polypeptide B3670, preferably represented by SEQ ID NO. 83630, or a homolog or fragment thereof, or if the activity of a polypeptide encoded by a nucleic acid molecule comprising the nucleic acid SEQ ID NO. 83629, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 83629 or polypeptide SEQ ID NO. 83630, respectively, or a homolog or a fragment thereof, is increased or generated, or if the activity acetolactate synthase is increased or generated in a non-human organism, like a microorganism or a plant cell, plant or part thereof, especially with plastidic localization, whereby the respective line discloses in column 7 tyrosine. For example, an increase of the tyrosine of at least 1 percent, particularly in a range of 60 to 97-percent is conferred as compared to a corresponding non-transformed wild type non-human organism.

Accordingly, in one embodiment, an increase of asparagine in a non-human organism, as compared to a corresponding non-transformed wild type non-human organism, is conferred in the process of the invention, if the activity of a polypeptide showing the activity of a aspartateammonia ligase, or if the activity of the polypeptide B3744, preferably represented by SEQ ID NO. 83658, or a homolog or fragment thereof, or if the activity of a polypeptide encoded by a nucleic acid molecule comprising the nucleic acid SEQ ID NO. 83657, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 83657 or polypeptide SEQ ID NO. 83658, respectively, or a homolog or a fragment thereof, is increased or generated, or if the activity aspartate-ammonia ligase is increased or generated in a non-human organism, like a microorganism or a plant cell, plant or part thereof, especially with plastidic localization, whereby the respective line discloses in column 7 asparagine. For example, an increase of the asparagine of at least 1 percent, particularly in a range of 36 to 2817-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a dihydroxyacid dehydratase, or if the activity of the polypeptide B3771, preferably represented by SEQ ID NO. 10253, or a homolog or fragment thereof, or if the activity of a polypeptide encoded by a nucleic acid molecule comprising the nucleic acid SEQ ID NO. 10252, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 10252 or polypeptide SEQ ID NO. 10253, respectively, or a homolog or a fragment thereof, is increased or generated, or if the activity dihydroxyacid dehydratase is increased or generated in a non-human organism, like a microorganism or a plant cell, plant or part thereof, especially with non-targeted localization, whereby the respective line discloses in column 7 glycine. For example, an increase of the glycine of at least 1 percent, particularly in a range of 39 to 114-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a dihydroxyacid dehydratase, or if the activity of the polypeptide B3771, preferably represented by SEQ ID NO. 10253, or a homolog or fragment thereof, or if the activity of a polypeptide encoded by a nucleic acid molecule comprising the nucleic acid SEQ ID NO. 10252, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 10252 or polypeptide SEQ ID NO. 10253, respectively, or a homolog or a fragment thereof, is increased or generated, or if the activity dihydroxyacid dehydratase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 69 to 81-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a b3791-protein, or if the activity of the polypeptide B3791, preferably represented by SEQ ID NO. 83727, or a homolog or fragment thereof, or if the activity of a polypeptide encoded by a nucleic acid molecule comprising the nucleic acid SEQ ID NO. 83726, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 83726 or polypeptide SEQ ID NO. 83727, respectively, or a homolog or a fragment thereof, is increased or generated, or if the activity b3791-protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 53 to 158-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a b3791-protein, or if the activity of the polypeptide B3791, preferably represented by SEQ ID NO. 83727, or a homolog or fragment thereof, or if the activity of a polypeptide encoded by a nucleic acid molecule comprising the nucleic acid SEQ ID NO. 83726, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 83726 or polypeptide SEQ ID NO. 83727, respectively, or a homolog or a fragment thereof, is increased or generated, or if the activity b3791-protein is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 39 to 117-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a enterobacterial common antigen polymerase, or if the activity of the polypeptide B3793, preferably represented by SEQ ID NO. 43801, or a homolog or fragment thereof, or if the activity of a polypeptide encoded by a nucleic acid molecule comprising the nucleic acid SEQ ID NO. 43800, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 43800 or polypeptide SEQ ID NO. 43801, respectively, or a homolog or a fragment thereof, is increased or generated, or if the activity enterobacterial common antigen polymerase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 26 to 438-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a DNA helicase II, or if the activity of the polypeptide B3813, preferably represented by SEQ ID NO. 43840, or a homolog or fragment thereof, or if the activity of a polypeptide encoded by a nucleic acid molecule comprising the nucleic acid SEQ ID NO. 43839, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 43839 or polypeptide SEQ ID NO. 43840, respectively, or a homolog or a fragment thereof, is increased or generated, or if the activity DNA helicase II is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 34 to 85-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a b3814-protein, or if the activity of the polypeptide B3814, preferably represented by SEQ ID NO. 44197, or a homolog or fragment thereof, or if the activity of a polypeptide encoded by a nucleic acid molecule comprising the nucleic acid SEQ ID NO. 44196, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 44196 or polypeptide SEQ ID NO. 44197, respectively, or a homolog or a fragment thereof, is increased or generated, or if the activity b3814-protein is increased or generated in a non-human organism, like a microorganism or a plant cell, plant or part thereof, especially with non-targeted localization, whereby the respective line discloses in column 7 citrulline. For example, an increase of the citrulline of at least 1 percent, particularly in a range of 30 to 124-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a transcriptional regulator, or if the activity of the polypeptide B3872, preferably represented by SEQ ID NO. 83822, or a homolog or fragment thereof, or if the activity of a polypeptide encoded by a nucleic acid molecule comprising the nucleic acid SEQ ID NO. 83821, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 83821 or polypeptide SEQ ID NO. 83822, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 20 to 44-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a transcriptional regulator, or if the activity of the polypeptide B3872, preferably represented by SEQ ID NO. 83822, or a homolog or fragment thereof, or if the activity of a polypeptide encoded by a nucleic acid molecule comprising the nucleic acid SEQ ID NO. 83821, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 83821 or polypeptide SEQ ID NO. 83822, respectively, or a homolog or a fragment thereof, is increased or generated, or if the activity transcriptional regulator is increased or generated in a non-human organism, like a microorganism or a plant cell, plant or part thereof, especially with plastidic localization, whereby the respective line discloses in column 7 aspartate. For example, an increase of the aspartate of at least 1 percent, particularly in a range of 55 to 96-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a transcriptional regulator, or if the activity of the polypeptide B3872, preferably represented by SEQ ID NO. 83822, or a homolog or fragment thereof, or if the activity of a polypeptide encoded by a nucleic acid molecule comprising the nucleic acid SEQ ID NO. 83821, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 83821 or polypeptide SEQ ID NO. 83822, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 36 to 59-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a transcriptional regulator, or if the activity of the polypeptide B3872, preferably represented by SEQ ID NO. 83822, or a homolog or fragment thereof, or if the activity of a polypeptide encoded by a nucleic acid molecule comprising the nucleic acid SEQ ID NO. 83821, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 83821 or polypeptide SEQ ID NO. 83822, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 35 to 54-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a b3989-protein, or if the activity of the polypeptide B3989, preferably represented by SEQ ID NO. 44373, or a homolog or fragment thereof, or if the activity of a polypeptide encoded by a nucleic acid molecule comprising the nucleic acid SEQ ID NO. 44372, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 44372 or polypeptide SEQ ID NO. 44373, respectively, or a homolog or a fragment thereof, is increased or generated, or if the activity b3989-protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 25 to 1035-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a b3989-protein, or if the activity of the polypeptide B3989, preferably represented by SEQ ID NO. 44373, or a homolog or fragment thereof, or if the activity of a polypeptide encoded by a nucleic acid molecule comprising the nucleic acid SEQ ID NO. 44372, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 44372 or polypeptide SEQ ID NO. 44373, respectively, or a homolog or a fragment thereof, is increased or generated, or if the activity b3989-protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 46 to 762-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a acetyltransferase, or if the activity of the polypeptide B4012, preferably represented by SEQ ID NO. 44379, or a homolog or fragment thereof, or if the activity of a polypeptide encoded by a nucleic acid molecule comprising the nucleic acid SEQ ID NO. 44378, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 44378 or polypeptide SEQ ID NO. 44379, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 51 to 282-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a acetyltransferase, or if the activity of the polypeptide B4012, preferably represented by SEQ ID NO. 44379, or a homolog or fragment thereof, or if the activity of a polypeptide encoded by a nucleic acid molecule comprising the nucleic acid SEQ ID NO. 44378, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 44378 or polypeptide SEQ ID NO. 44379, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 33 to 47-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a aspartate kinase, or if the activity of the polypeptide B4024, preferably represented by SEQ ID NO. 83836, or a homolog or fragment thereof, or if the activity of a polypeptide encoded by a nucleic acid molecule comprising the nucleic acid SEQ ID NO. 83835, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 83835 or polypeptide SEQ ID NO. 83836, respectively, or a homolog or a fragment thereof, is increased or generated, or if the activity aspartate kinase is increased or generated in a non-human organism, like a microorganism or a plant cell, plant or part thereof, especially with plastidic localization, whereby the respective line discloses in column 7 glycine. For example, an increase of the glycine of at least 1 percent, partitularly in a range of 210 to 262-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a aspartate kinase, or if the activity of the polypeptide B4024, preferably represented by SEQ ID NO. 83836, or a homolog or fragment thereof, or if the activity of a polypeptide encoded by a nucleic acid molecule comprising the nucleic acid SEQ ID NO. 83835, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 83835 or polypeptide SEQ ID NO. 83836, respectively, or a homolog or a fragment thereof, is increased or generated, or if the activity aspartate kinase is increased or generated in a non-human organism, like a microorganism or a plant cell, plant or part thereof, especially with plastidic localization, whereby the respective line discloses in column 7 serine. For example, an increase of the serine of at least 1 percent, particularly in a range of 35 to 399-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a b4029-protein, or if the activity of the polypeptide B4029, preferably represented by SEQ ID NO. 10741, or a homolog or fragment thereof, or if the activity of a polypeptide encoded by a nucleic acid molecule comprising the nucleic acid SEQ ID NO. 10740, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 10740 or polypeptide SEQ ID NO. 10741, respectively, or a homolog or a fragment thereof, is increased or generated, or if the activity b4029-protein is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 22 to 775-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a b4029-protein, or if the activity of the polypeptide B4029, preferably represented by SEQ ID NO. 10741, or a homolog or fragment thereof, or if the activity of a polypeptide encoded by a nucleic acid molecule comprising the nucleic acid SEQ ID NO. 10740, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 10740 or polypeptide SEQ ID NO. 10741, respectively, or a homolog or a fragment thereof, is increased or generated, or if the activity b4029-protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 59 to 231-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a b4029-protein, or if the activity of the polypeptide B4029, preferably represented by SEQ ID NO. 10741, or a homolog or fragment thereof, or if the activity of a polypeptide encoded by a nucleic acid molecule comprising the nucleic acid SEQ ID NO. 10740, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 10740 or polypeptide SEQ ID NO. 10741, respectively, or a homolog or a fragment thereof, is increased or generated, or if the activity b4029-protein is increased or generated in a non-human organism, like a microorganism or a plant cell, plant or part thereof, especially with non-targeted localization, whereby the respective line discloses in column 7 glycine. For example, an increase of the glycine of at least 1 percent, particularly in a range of 39 to 590-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a b4029-protein, or if the activity of the polypeptide B4029, preferably represented by SEQ ID NO. 10741, or a homolog or fragment thereof, or if the activity of a polypeptide encoded by a nucleic acid molecule comprising the nucleic acid SEQ ID NO. 10740, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 10740 or polypeptide SEQ ID NO. 10741, respectively, or a homolog or a fragment thereof, is increased or generated, or if the activity b4029-protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 28 to 2010-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a b4029-protein, or if the activity of the polypeptide B4029, preferably represented by SEQ ID NO. 10741, or a homolog or fragment thereof, or if the activity of a polypeptide encoded by a nucleic acid molecule comprising the nucleic acid SEQ ID NO. 10740, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 10740 or polypeptide SEQ ID NO. 10741, respectively, or a homolog or a fragment thereof, is increased or generated, or if the activity b4029-protein is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 28 to 177-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a b4029-protein, or if the activity of the polypeptide B4029, preferably represented by SEQ ID NO. 10741, or a homolog or fragment thereof, or if the activity of a polypeptide encoded by a nucleic acid molecule comprising the nucleic acid SEQ ID NO. 10740, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 10740 or polypeptide SEQ ID NO. 10741, respectively, or a homolog or a fragment thereof, is increased or generated, or if the activity b4029-protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 36 to 3270-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a b4029-protein, or if the activity of the polypeptide B4029, preferably represented by SEQ ID NO. 10741, or a homolog or fragment thereof, or if the activity of a polypeptide encoded by a nucleic acid molecule comprising the nucleic acid SEQ ID NO. 10740, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 10740 or polypeptide SEQ ID NO. 10741, respectively, or a homolog or a fragment thereof, is increased or generated, or if the activity b4029-protein is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 44 to 367-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a b4121-protein, or if the activity of the polypeptide B4121, preferably represented by SEQ ID NO. 44610, or a homolog or fragment thereof, or if the activity of a polypeptide encoded by a nucleic acid molecule comprising the nucleic acid SEQ ID NO. 44609, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 44609 or polypeptide SEQ ID NO. 44610, respectively, or a homolog or a fragment thereof, is increased or generated, or if the activity b4121-protein is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 23 to 92-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a acetyltransferase, or if the activity of the polypeptide B4256, preferably represented by SEQ ID NO. 45322, or a homolog or fragment thereof, or if the activity of a polypeptide encoded by a nucleic acid molecule comprising the nucleic acid SEQ ID NO. 45321, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 45321 or polypeptide SEQ ID NO. 45322, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 56 to 155-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a acetyltransferase, or if the activity of the polypeptide B4256, preferably represented by SEQ ID NO. 45322, or a homolog or fragment thereof, or if the activity of a polypeptide encoded by a nucleic acid molecule comprising the nucleic acid SEQ ID NO. 45321, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 45321 or polypeptide SEQ ID NO. 45322, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 29 to 242-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a acetyltransferase, or if the activity of the polypeptide B4256, preferably represented by SEQ ID NO. 45322, or a homolog or fragment thereof, or if the activity of a polypeptide encoded by a nucleic acid molecule comprising the nucleic acid SEQ ID NO. 45321, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 45321 or polypeptide SEQ ID NO. 45322, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 31 to 227-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a acetyltransferase, or if the activity of the polypeptide B4256, preferably represented by SEQ ID NO. 45322, or a homolog or fragment thereof, or if the activity of a polypeptide encoded by a nucleic acid molecule comprising the nucleic acid SEQ ID NO. 45321, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 45321 or polypeptide SEQ ID NO. 45322, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 151 to 280-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a thioredoxin, or if the activity of the polypeptide C_pp004096192r, preferably represented by SEQ ID NO. 45758, or a homolog or fragment thereof, or if the activity of a polypeptide encoded by a nucleic acid molecule comprising the nucleic acid SEQ ID NO. 45757, preferably the coding region thereof, or a homolog or fragment thereof, e.g. derived from Physcomitrella patens, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 45757 or polypeptide SEQ ID NO. 45758, respectively, or a homolog or a fragment thereof, is increased or generated, or if the activity thioredoxin is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 32 to 75-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a lysine decarboxylase, or if the activity of the polypeptide D90900, preferably represented by SEQ ID NO. 84080, or a homolog or fragment thereof, or if the activity of a polypeptide encoded by a nucleic acid molecule comprising the nucleic acid SEQ ID NO. 84079, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 84079 or polypeptide SEQ ID NO. 84080, respectively, or a homolog or a fragment thereof, is increased or generated, or if the activity lysine decarboxylase is increased or generated in a non-human organism, like a microorganism or a plant cell, plant or part thereof, especially with plastidic localization, whereby the respective line discloses in column 7 alanine. For example, an increase of the alanine of at least 1 percent, particularly in a range of 20 to 34-percent is conferred as compared to a corresponding non-transformed wild type non-human organism.

Accordingly, in one embodiment, an increase of asparagine in a non-human organism, as compared to a corresponding non-transformed wild type non-human organism, is conferred in the process of the invention, if the activity of a polypeptide showing the activity of a GM02LC11114-protein, or if the activity of the polypeptide GM02LC11114, preferably represented by SEQ ID NO. 45796, or a homolog or fragment thereof, or if the activity of a polypeptide encoded by a nucleic acid molecule comprising the nucleic acid SEQ ID NO. 45795, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 45795 or polypeptide SEQ ID NO. 45796, respectively, or a homolog or a fragment thereof, is increased or generated, or if the activity GM02LC11114-protein is increased or generated in a non-human organism, like a microorganism or a plant cell, plant or part thereof, especially with non-targeted localization, whereby the respective line discloses in column 7 asparagine. For example, an increase of the asparagine of at least 1 percent, particularly in a range of 78 to 249-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a GM02LC11114-protein, or if the activity of the polypeptide GM02LC11114, preferably represented by SEQ ID NO. 45796, or a homolog or fragment thereof, or if the activity of a polypeptide encoded by a nucleic acid molecule comprising the nucleic acid SEQ ID NO. 45795, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 45795 or polypeptide SEQ ID NO. 45796, respectively, or a homolog or a fragment thereof, is increased or generated, or if the activity GM02LC11114-protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 44 to 76-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a glutaredoxin, or if the activity of the polypeptide GM02LC12622, preferably represented by SEQ ID NO. 10812, or a homolog or fragment thereof, or if the activity of a polypeptide encoded by a nucleic acid molecule comprising the nucleic acid SEQ ID NO. 10811, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 10811 or polypeptide SEQ ID NO. 10812, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 21 to 46-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a glutaredoxin, or if the activity of the polypeptide GM02LC12622, preferably represented by SEQ ID NO. 10812, or a homolog or fragment thereof, or if the activity of a polypeptide encoded by a nucleic acid molecule comprising the nucleic acid SEQ ID NO. 10811, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 10811 or polypeptide SEQ ID NO. 10812, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 44 to 218-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a glutaredoxin, or if the activity of the polypeptide GM02LC12622, preferably represented by SEQ ID NO. 10812, or a homolog or fragment thereof, or if the activity of a polypeptide encoded by a nucleic acid molecule comprising the nucleic acid SEQ ID NO. 10811, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 10811 or polypeptide SEQ ID NO. 10812, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 45 to 525-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a histone H2A, or if the activity of the polypeptide GM02LC15313, preferably represented by SEQ ID NO. 45898, or a homolog or fragment thereof, or if the activity of a polypeptide encoded by a nucleic acid molecule comprising the nucleic acid SEQ ID NO. 45897, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 45897 or polypeptide SEQ ID NO. 45898, respectively, or a homolog or a fragment thereof, is increased or generated, or if the activity histone H2A is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 32 to 137-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a histone H2A, or if the activity of the polypeptide GM02LC15313, preferably represented by SEQ ID NO. 45898, or a homolog or fragment thereof, or if the activity of a polypeptide encoded by a nucleic acid molecule comprising the nucleic acid SEQ ID NO. 45897, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 45897 or polypeptide SEQ ID NO. 45898, respectively, or a homolog or a fragment thereof, is increased or generated, or if the activity histone H2A is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 53 to 84-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a GM02LC17485-protein, or if the activity of the polypeptide GM02LC17485, preferably represented by SEQ ID NO. 46406, or a homolog or fragment thereof, or if the activity of a polypeptide encoded by a nucleic acid molecule comprising the nucleic acid SEQ ID NO. 46405, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 46405 or polypeptide SEQ ID NO. 46406, respectively, or a homolog or a fragment thereof, is increased or generated, or if the activity GM02LC17485-protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 57 to 118-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a RNA binding protein, or if the activity of the polypeptide GM02LC17556, preferably represented by SEQ ID NO. 46516, or a homolog or fragment thereof, or if the activity of a polypeptide encoded by a nucleic acid molecule comprising the nucleic acid SEQ ID NO. 46515, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 46515 or polypeptide SEQ ID NO. 46516, respectively, or a homolog or a fragment thereof, is increased or generated, or if the activity RNA binding protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 26 to 179-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a RNA binding protein, or if the activity of the polypeptide GM02LC17556, preferably represented by SEQ ID NO. 46516, or a homolog or fragment thereof, or if the activity of a polypeptide encoded by a nucleic acid molecule comprising the nucleic acid SEQ ID NO. 46515, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 46515 or polypeptide SEQ ID NO. 46516, respectively, or a homolog or a fragment thereof, is increased or generated, or if the activity RNA binding protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 48 to 179-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a transcription factor, or if the activity of the polypeptide GM02LC21368, preferably represented by SEQ ID NO. 84199, or a homolog or fragment thereof, or if the activity of a polypeptide encoded by a nucleic acid molecule comprising the nucleic acid SEQ ID NO. 84198, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 84198 or polypeptide SEQ ID NO. 84199, respectively, or a homolog or a fragment thereof, is increased or generated, or if the activity transcription factor is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 59 to 383-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a transcription factor, or if the activity of the polypeptide GM02LC21368, preferably represented by SEQ ID NO. 84199, or a homolog or fragment thereof, or if the activity of a polypeptide encoded by a nucleic acid molecule comprising the nucleic acid SEQ ID NO. 84198, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 84198 or polypeptide SEQ ID NO. 84199, respectively, or a homolog or a fragment thereof, is increased or generated, or if the activity transcription factor is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 26 to 82-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a transcription factor, or if the activity of the polypeptide GM02LC21368, preferably represented by SEQ ID NO. 84199, or a homolog or fragment thereof, or if the activity of a polypeptide encoded by a nucleic acid molecule comprising the nucleic acid SEQ ID NO. 84198, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 84198 or polypeptide SEQ ID NO. 84199, respectively, or a homolog or a fragment thereof, is increased or generated, or if the activity transcription factor is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 36 to 903-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a cyclin D, or if the activity of the polypeptide GM02LC44512, preferably represented by SEQ ID NO. 46851, or a homolog or fragment thereof, or if the activity of a polypeptide encoded by a nucleic acid molecule comprising the nucleic acid SEQ ID NO. 46850, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 46850 or polypeptide SEQ ID NO. 46851, respectively, or a homolog or a fragment thereof, is increased or generated, or if the activity cyclin D is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 29 to 66-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a cyclin D, or if the activity of the polypeptide GM02LC44512, preferably represented by SEQ ID NO. 46851, or a homolog or fragment thereof, or if the activity of a polypeptide encoded by a nucleic acid molecule comprising the nucleic acid SEQ ID NO. 46850, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 46850 or polypeptide SEQ ID NO. 46851, respectively, or a homolog or a fragment thereof, is increased or generated, or if the activity cyclin D is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 37 to 67-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a glutaredoxin, or if the activity of the polypeptide NZ_AAAU02000016.150, preferably represented by SEQ ID NO. 47160, or a homolog or fragment thereof, or if the activity of a polypeptide encoded by a nucleic acid molecule comprising the nucleic acid SEQ ID NO. 47159, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 47159 or polypeptide SEQ ID NO. 47160, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 21 to 74-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a glutaredoxin, or if the activity of the polypeptide NZ_AAAU02000016.150, preferably represented by SEQ ID NO. 47160, or a homolog or fragment thereof, or if the activity of a polypeptide encoded by a nucleic acid molecule comprising the nucleic acid SEQ ID NO. 47159, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 47159 or polypeptide SEQ ID NO. 47160, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 glycine. For example, an increase of the glycine of at least 1 percent, particularly in a range of 40 to 103-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a glutaredoxin, or if the activity of the polypeptide NZ_AAAU02000016.150, preferably represented by SEQ ID NO. 47160, or a homolog or fragment thereof, or if the activity of a polypeptide encoded by a nucleic acid molecule comprising the nucleic acid SEQ ID NO. 47159, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 47159 or polypeptide SEQ ID NO. 47160, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 24 to 65-percent is conferred as compared to a corresponding non-transformed wild type non-human organism.

Accordingly, in one embodiment, an increase of asparagine in a non-human organism, as compared to a corresponding non-transformed wild type non-human organism, is conferred in the process of the invention, if the activity of a polypeptide showing the activity of a s_pp015018333r-protein, or if the activity of the polypeptide S_pp015018333r, preferably represented by SEQ ID NO. 59852, or a homolog or fragment thereof, or if the activity of a polypeptide encoded by a nucleic acid molecule comprising the nucleic acid SEQ ID NO. 59851, preferably the coding region thereof, or a homolog or fragment thereof, e.g. derived from Physcomitrella patens, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 59851 or polypeptide SEQ ID NO. 59852, respectively, or a homolog or a fragment thereof, is increased or generated, or if the activity s_pp015018333r-protein is increased or generated in a non-human organism, like a microorganism or a plant cell, plant or part thereof, especially with non-targeted localization, whereby the respective line discloses in column 7 asparagine. For example, an increase of the asparagine of at least 1 percent, particularly in a range of 43 to 143-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a s_pp015018333r-protein, or if the activity of the polypeptide S_pp015018333r, preferably represented by SEQ ID NO. 59852, or a homolog or fragment thereof, or if the activity of a polypeptide encoded by a nucleic acid molecule comprising the nucleic acid SEQ ID NO. 59851, preferably the coding region thereof, or a homolog or fragment thereof, e.g. derived from Physcomitrella patens, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 59851 or polypeptide SEQ ID NO. 59852, respectively, or a homolog or a fragment thereof, is increased or generated, or if the activity s_pp015018333r-protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 37 to 174-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a s_pp015018333r-protein, or if the activity of the polypeptide S_pp015018333r, preferably represented by SEQ ID NO. 59852, or a homolog or fragment thereof, or if the activity of a polypeptide encoded by a nucleic acid molecule comprising the nucleic acid SEQ ID NO. 59851, preferably the coding region thereof, or a homolog or fragment thereof, e.g. derived from Physcomitrella patens, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 59851 or polypeptide SEQ ID NO. 59852, respectively, or a homolog or a fragment thereof, is increased or generated, or if the activity s_pp015018333r-protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 41 to 102-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a s_pp015018333r-protein, or if the activity of the polypeptide S_pp015018333r, preferably represented by SEQ ID NO. 59852, or a homolog or fragment thereof, or if the activity of a polypeptide encoded by a nucleic acid molecule comprising the nucleic acid SEQ ID NO. 59851, preferably the coding region thereof, or a homolog or fragment thereof, e.g. derived from Physcomitrella patens, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 59851 or polypeptide SEQ ID NO. 59852, respectively, or a homolog or a fragment thereof, is increased or generated, or if the activity s_pp015018333r-protein is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 45 to 114 percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a N-acetyl-gamma-glutamyl-phosphate reductase, or if the activity of the polypeptide Sll0080, preferably represented by SEQ ID NO. 47567, or a homolog or fragment thereof, or if the activity of a polypeptide encoded by a nucleic acid molecule comprising the nucleic acid SEQ ID NO. 47566, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 47566 or polypeptide SEQ ID NO. 47567, respectively, or a homolog or a fragment thereof, is increased or generated, or if the activity N-acetyl-gamma-glutamyl-phosphate reductase is increased or generated in a non-human organism, like a microorganism or a plant cell, plant or part thereof, especially with plastidic localization, whereby the respective line discloses in column 7 citrulline. For example, an increase of the citrulline of at least 1 percent, particularly in a range of 31 to 60-percent is conferred as compared to a corresponding non-transformed wild type non-human organism.

Accordingly, in one embodiment, an increase of asparagine in a non-human organism, as compared to a corresponding non-transformed wild type non-human organism, is conferred in the process of the invention, if the activity of a polypeptide showing the activity of a agmatinase, or if the activity of the polypeptide Sll0228, preferably represented by SEQ ID NO. 84266, or a homolog or fragment thereof, or if the activity of a polypeptide encoded by a nucleic acid molecule comprising the nucleic acid SEQ ID NO. 84265, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 84265 or polypeptide SEQ ID NO. 84266, respectively, or a homolog or a fragment thereof, is increased or generated, or if the activity agmatinase is increased or generated in a non-human organism, like a microorganism or a plant cell, plant or part thereof, especially with plastidic localization, whereby the respective line discloses in column 7 asparagine. For example, an increase of the asparagine of at least 1 percent, particularly in a range of 76 to 319-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a agmatinase, or if the activity of the polypeptide Sll0228, preferably represented by SEQ ID NO. 84266, or a homolog or fragment thereof, or if the activity of a polypeptide encoded by a nucleic acid molecule comprising the nucleic acid SEQ ID NO. 84265, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 84265 or polypeptide SEQ ID NO. 84266, respectively, or a homolog or a fragment thereof, is increased or generated, or if the activity agmatinase is increased or generated in a non-human organism, like a microorganism or a plant cell, plant or part thereof, especially with plastidic localization, whereby the respective line discloses in column 7 tyrosine. For example, an increase of the tyrosine of at least 1 percent, particularly in a range of 48 to 58-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a ABC transporter ATP binding component, or if the activity of the polypeptide Sll0240, preferably represented by SEQ ID NO. 84442, or a homolog or fragment thereof, or if the activity of a polypeptide encoded by a nucleic acid molecule comprising the nucleic acid SEQ ID NO. 84441, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table 1,11 or IV, application No. 3, column 5 or 8 in the respective same line as the nucleic acid molecule SEQ ID NO. 84441 or polypeptide SEQ ID NO. 84442, respectively, or a homolog or a fragment thereof, is increased or generated, or if the activity ABC transporter ATP binding component is increased or generated in a non-human organism, like a microorganism or a plant cell, plant or part thereof, especially with plastidic localization, whereby the respective line discloses in column 7 alanine. For example, an increase of the alanine of at least 1 percent, particularly in a range of 30 to 40-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a flavodoxin, or if the activity of the polypeptide Sll0248, preferably represented by SEQ ID NO. 49144, or a homolog or fragment thereof, or if the activity of a polypeptide encoded by a nucleic acid molecule comprising the nucleic acid SEQ ID NO. 49143, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 49143 or polypeptide SEQ ID NO. 49144, respectively, or a homolog or a fragment thereof, is increased or generated, or if the activity flavodoxin is increased or generated in a non-human organism, like a microorganism or a plant cell, plant or part thereof, especially with plastidic localization, whereby the respective line discloses in column 7 phenylalanine. For example, an increase of the phenylalanine of at least 1 percent, particularly in a range of 33 to 128-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a flavodoxin, or if the activity of the polypeptide Sll0248, preferably represented by SEQ ID NO. 49144, or a homolog or fragment thereof, or if the activity of a polypeptide encoded by a nucleic acid molecule comprising the nucleic acid SEQ ID NO. 49143, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 49143 or polypeptide SEQ ID NO. 49144, respectively, or a homolog or a fragment thereof, is increased or generated, or if the activity flavodoxin is increased or generated in a non-human organism, like a microorganism or a plant cell, plant or part thereof, especially with plastidic localization, whereby the respective line discloses in column 7 tyrosine. For example, an increase of the tyrosine of at least 1 percent, particularly in a range of 40 to 277-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a sll0281-protein, or if the activity of the polypeptide Sll0281, preferably represented by SEQ ID NO. 84866, or a homolog or fragment thereof, or if the activity of a polypeptide encoded by a nucleic acid molecule comprising the nucleic acid SEQ ID NO. 84865, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 84865 or polypeptide SEQ ID NO. 84866, respectively, or a homolog or a fragment thereof, is increased or generated, or if the activity sll0281-protein is increased or generated in a non-human organism, like a microorganism or a plant cell, plant or part thereof, especially with plastidic localization, whereby the respective line discloses in column 7 alanine. For example, an increase of the alanine of at least 1 percent, particularly in a range of 22 to 34-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a uroporphyrin-III C-methyltransferase, or if the activity of the polypeptide Sll0378, preferably represented by SEQ ID NO. 84870, or a homolog or fragment thereof, or if the activity of a polypeptide encoded by a nucleic acid molecule comprising the nucleic acid SEQ ID NO. 84869, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 84869 or polypeptide SEQ ID NO. 84870, respectively, or a homolog or a fragment thereof, is increased or generated, or if the activity uroporphyrin-III C-methyltransferase is increased or generated in a non-human organism, like a microorganism or a plant cell, plant or part thereof, especially with plastidic localization, whereby the respective line discloses in column 7 serine. For example, an increase of the serine of at least 1 percent, particularly in a range of 24 to 126-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a urease subunit, or if the activity of the polypeptide Sll0420, preferably represented by SEQ ID NO. 50105, or a homolog or fragment thereof, or if the activity of a polypeptide encoded by a nucleic acid molecule comprising the nucleic acid SEQ ID NO. 50104, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 50104 or polypeptide SEQ ID NO. 50105, respectively, or a homolog or a fragment thereof, is increased or generated, or if the activity urease subunit is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 31 to 49-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a urease subunit, or if the activity of the polypeptide Sll0420, preferably represented by SEQ ID NO. 50105, or a homolog or fragment thereof, or if the activity of a polypeptide encoded by a nucleic acid molecule comprising the nucleic acid SEQ ID NO. 50104, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 50104 or polypeptide SEQ ID NO. 50105, respectively, or a homolog or a fragment thereof, is increased or generated, or if the activity urease subunit is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 36 to 94-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a glucokinase, or if the activity of the polypeptide Sll0593, preferably represented by SEQ ID NO. 85511, or a homolog or fragment thereof, or if the activity of a polypeptide encoded by a nucleic acid molecule comprising the nucleic acid SEQ ID NO. 85510, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 85510 or polypeptide SEQ ID NO. 85511, respectively, or a homolog or a fragment thereof, is increased or generated, or if the activity glucokinase is increased or generated in a non-human organism, like a microorganism or a plant cell, plant or part thereof, especially with plastidic localization, whereby the respective line discloses in column 7 citrulline. For example, an increase of the citrulline of at least 1 percent, particularly in a range of 54 to 140-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a permease protein of phosphate ABC transporter, or if the activity of the polypeptide Sll0682, preferably represented by SEQ ID NO. 50951, or a homolog or fragment thereof, or if the activity of a polypeptide encoded by a nucleic acid molecule comprising the nucleic acid SEQ ID NO. 50950, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 50950 or polypeptide SEQ ID NO. 50951, respectively, or a homolog or a fragment thereof, is increased or generated, or if the activity permease protein of phosphate ABC transporter is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 26 to 90-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a permease protein of phosphate ABC transporter, or if the activity of the polypeptide Sll0682, preferably represented by SEQ ID NO. 50951, or a homolog or fragment thereof, or if the activity of a polypeptide encoded by a nucleic acid molecule comprising the nucleic acid SEQ ID NO. 50950, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 50950 or polypeptide SEQ ID NO. 50951, respectively, or a homolog or a fragment thereof, is increased or generated, or if the activity permease protein of phosphate ABC transporter is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 34 to 92-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a oxireductase, or if the activity of the polypeptide Sll0816, preferably represented by SEQ ID NO. 51199, or a homolog or fragment thereof, or if the activity of a polypeptide encoded by a nucleic acid molecule comprising the nucleic acid SEQ ID NO. 51198, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 51198 or polypeptide SEQ ID NO. 51199, respectively, or a homolog or a fragment thereof, is increased or generated, or if the activity oxireductase is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 52 to 318-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a oxireductase, or if the activity of the polypeptide Sll0816, preferably represented by SEQ ID NO. 51199, or a homolog or fragment thereof, or if the activity of a polypeptide encoded by a nucleic acid molecule comprising the nucleic acid SEQ ID NO. 51198, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 51198 or polypeptide SEQ ID NO. 51199, respectively, or a homolog or a fragment thereof, is increased or generated, or if the activity oxireductase is increased or generated in a non-human organism, like a microorganism or a plant cell, plant or part thereof, especially with non-targeted localization, whereby the respective line discloses in column 7 glycine. For example, an increase of the glycine of at least 1 percent, particularly in a range of 37 to 110-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a oxireductase, or if the activity of the polypeptide Sll0816, preferably represented by SEQ ID NO. 51199, or a homolog or fragment thereof, or if the activity of a polypeptide encoded by a nucleic acid molecule comprising the nucleic acid SEQ ID NO. 51198, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 51198 or polypeptide SEQ ID NO. 51199, respectively, or a homolog or a fragment thereof, is increased or generated, or if the activity oxireductase is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 32 to 198-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a oxireductase, or if the activity of the polypeptide Sll0816, preferably represented by SEQ ID NO. 51199, or a homolog or fragment thereof, or if the activity of a polypeptide encoded by a nucleic acid molecule comprising the nucleic acid SEQ ID NO. 51198, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 51198 or polypeptide SEQ ID NO. 51199, respectively, or a homolog or a fragment thereof, is increased or generated, or if the activity oxireductase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 53 to 88-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a malate dehydrogenase, or if the activity of the polypeptide Sll0891, preferably represented by SEQ ID NO. 51269, or a homolog or fragment thereof, or if the activity of a polypeptide encoded by a nucleic acid molecule comprising the nucleic acid SEQ ID NO. 51268, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 51268 or polypeptide SEQ ID NO. 51269, respectively, or a homolog or a fragment thereof, is increased or generated, or if the activity malate dehydrogenase is increased or generated in a non-human organism, like a microorganism or a plant cell, plant or part thereof, especially with plastidic localization, whereby the respective line discloses in column 7 aspartate. For example, an increase of the aspartate of at least 1 percent, particularly in a range of 90 to 176-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a malate dehydrogenase, or if the activity of the polypeptide Sll0891, preferably represented by SEQ ID NO. 51269, or a homolog or fragment thereof, or if the activity of a polypeptide encoded by a nucleic acid molecule comprising the nucleic acid SEQ ID NO. 51268, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 51268 or polypeptide SEQ ID NO. 51269, respectively, or a homolog or a fragment thereof, is increased or generated, or if the activity malate dehydrogenase is increased or generated in a non-human organism, like a microorganism or a plant cell, plant or part thereof, especially with plastidic localization, whereby the respective line discloses in column 7 phenylalanine. For example, an increase of the phenylalanine of at least 1 percent, particularly in a range of 37 to 118-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a malate dehydrogenase, or if the activity of the polypeptide Sll0891, preferably represented by SEQ ID NO. 51269, or a homolog or fragment thereof, or if the activity of a polypeptide encoded by a nucleic acid molecule comprising the nucleic acid SEQ ID NO. 51268, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 51268 or polypeptide SEQ ID NO. 51269, respectively, or a homolog or a fragment thereof, is increased or generated, or if the activity malate dehydrogenase is increased or generated in a non-human organism, like a microorganism or a plant cell, plant or part thereof, especially with plastidic localization, whereby the respective line discloses in column 7 serine. For example, an increase of the serine of at least 1 percent, particularly in a range of 23 to 95-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a 3-deoxy-7-phosphoheptulonate synthase, or if the activity of the polypeptide Sll0934, preferably represented by SEQ ID NO. 52247, or a homolog or fragment thereof, or if the activity of a polypeptide encoded by a nucleic acid molecule comprising the nucleic acid SEQ ID NO. 52246, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 52246 or polypeptide SEQ ID NO. 52247, respectively, or a homolog or a fragment thereof, is increased or generated, or if the activity 3-deoxy-7-phosphoheptulonate synthase is increased or generated in a non-human organism, like a microorganism or a plant cell, plant or part thereof, especially with mitochondrial localization, whereby the respective line discloses in column 7 aspartate. For example, an increase of the aspartate of at least 1 percent, particularly in a range of 61 to 94-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a glycogen synthase, or if the activity of the polypeptide Sll0945, preferably represented by SEQ ID NO. 52365, or a homolog or fragment thereof, or if the activity of a polypeptide encoded by a nucleic acid molecule comprising the nucleic acid SEQ ID NO. 52364, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 52364 or polypeptide SEQ ID NO. 52365, respectively, or a homolog or a fragment thereof, is increased or generated, or if the activity glycogen synthase is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 28 to 86-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a carbon dioxide concentrating mechanism protein, or if the activity of the polypeptide Sll1031, preferably represented by SEQ ID NO. 52635, or a homolog or fragment thereof, or if the activity of a polypeptide encoded by a nucleic acid molecule comprising the nucleic acid SEQ ID NO. 52634, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 52634 or polypeptide SEQ ID NO. 52635, respectively, or a homolog or a fragment thereof, is increased or generated, or if the activity carbon dioxide concentrating mechanism protein is increased or generated in a non-human organism, like a microorganism or a plant cell, plant or part thereof, especially with mitochondrial localization, whereby the respective line discloses in column 7 aspartate. For example, an increase of the aspartate of at least 1 percent, particularly in a range of 61 to 317-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a carbon dioxide concentrating mechanism protein, or if the activity of the polypeptide Sll1031, preferably represented by SEQ ID NO. 52635, or a homolog or fragment thereof, or if the activity of a polypeptide encoded by a nucleic acid molecule comprising the nucleic acid SEQ ID NO. 52634, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 52634 or polypeptide SEQ ID NO. 52635, respectively, or a homolog or a fragment thereof, is increased or generated, or if the activity carbon dioxide concentrating mechanism protein is increased or generated in a non-human organism, like a microorganism or a plant cell, plant or part thereof, especially with mitochondrial localization, whereby the respective line discloses in column 7 phenylalanine. For example, an increase of the phenylalanine of at least 1 percent, particularly in a range of 33 to 128-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a carbon dioxide concentrating mechanism protein, or if the activity of the polypeptide Sll1031, preferably represented by SEQ ID NO. 52635, or a homolog or fragment thereof, or if the activity of a polypeptide encoded by a nucleic acid molecule comprising the nucleic acid SEQ ID NO. 52634, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 52634 or polypeptide SEQ ID NO. 52635, respectively, or a homolog or a fragment thereof, is increased or generated, or if the activity carbon dioxide concentrating mechanism protein is increased or generated in a non-human organism, like a microorganism or a plant cell, plant or part thereof, especially with mitochondrial localization, whereby the respective line discloses in column 7 serine. For example, an increase of the serine of at least 1 percent, particularly in a range of 24 to 67 percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a sll1032-protein, or if the activity of the polypeptide Sll1032, preferably represented by SEQ ID NO. 85724, or a homolog or fragment thereof, or if the activity of a polypeptide encoded by a nucleic acid molecule comprising the nucleic acid SEQ ID NO. 85723, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 85723 or polypeptide SEQ ID NO. 85724, respectively, or a homolog or a fragment thereof, is increased or generated, or if the activity sll1032-protein is increased or generated in a non-human organism, like a microorganism or a plant cell, plant or part thereof, especially with mitochondrial localization, whereby the respective line discloses in column 7 aspartate. For example, an increase of the aspartate of at least 1 percent, particularly in a range of 45 to 106-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a phosphoribosylformyl glycinamidine synthase subunit, or if the activity of the polypeptide Sll1056, preferably represented by SEQ ID NO. 52661, or a homolog or fragment thereof, or if the activity of a polypeptide encoded by a nucleic acid molecule comprising the nucleic acid SEQ ID NO. 52660, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 52660 or polypeptide SEQ ID NO. 52661, respectively, or a homolog or a fragment thereof, is increased or generated, or if the activity phosphoribosylformyl glycinamidine synthase subunit is increased or generated in a non-human organism, like a microorganism or a plant cell, plant or part thereof, especially with plastidic localization, whereby the respective line discloses in column 7 serine. For example, an increase of the serine of at least 1 percent, particularly in a range of 24 to 75-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a 5′-nucleotidase, or if the activity of the polypeptide Sll1108, preferably represented by SEQ ID NO. 11212, or a homolog or fragment thereof, or if the activity of a polypeptide encoded by a nucleic acid molecule comprising the nucleic acid SEQ ID NO. 11211, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 11211 or polypeptide SEQ ID NO. 11212, respectively, or a homolog or a fragment thereof, is increased or generated, or if the activity 5′-nucleotidase is increased or generated in a non-human organism, like a microorganism or a plant cell, plant or part thereof, especially with mitochondrial localization, whereby the respective line discloses in column 7 aspartate. For example, an increase of the aspartate of at least 1 percent, particularly in a range of 55 to 137-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a 5′-nucleotidase, or if the activity of the polypeptide Sll1108, preferably represented by SEQ ID NO. 11212, or a homolog or fragment thereof, or if the activity of a polypeptide encoded by a nucleic acid molecule comprising the nucleic acid SEQ ID NO. 11211, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 11211 or polypeptide SEQ ID NO. 11212, respectively, or a homolog or a fragment thereof, is increased or generated, or if the activity 5′-nucleotidase is increased or generated in a non-human organism, like a microorganism or a plant cell, plant or part thereof, especially with mitochondrial localization, whereby the respective line discloses in column 7 5-oxoproline. For example, an increase of the 5-oxoproline of at least 1 percent, particularly in a range of 39 to 103-percent is conferred as compared to a corresponding non-transformed wild type non-human organism.

Accordingly, in one embodiment, an increase of asparagine in a non-human organism, as compared to a corresponding non-transformed wild type non-human organism, is conferred in the process of the invention, if the activity of a polypeptide showing the activity of a glycogen (starch) synthase, or if the activity of the polypeptide Sll1393, preferably represented by SEQ ID NO. 53457, or a homolog or fragment thereof, or if the activity of a polypeptide encoded by a nucleic acid molecule comprising the nucleic acid SEQ ID NO. 53456, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 53456 or polypeptide SEQ ID NO. 53457, respectively, or a homolog or a fragment thereof, is increased or generated, or if the activity glycogen (starch) synthase is increased or generated in a non-human organism, like a microorganism or a plant cell, plant or part thereof, especially with non-targeted localization, whereby the respective line discloses in column 7 asparagine. For example, an increase of the asparagine of at least 1 percent, particularly in a range of 55 to 264-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a glycogen (starch) synthase, or if the activity of the polypeptide Sll1393, preferably represented by SEQ ID NO. 53457, or a homolog or fragment thereof, or if the activity of a polypeptide encoded by a nucleic acid molecule comprising the nucleic acid SEQ ID NO. 53456, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 53456 or polypeptide SEQ ID NO. 53457, respectively, or a homolog or a fragment thereof, is increased or generated, or if the activity glycogen (starch) synthase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 82 to 129-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a CTP synthetase, or if the activity of the polypeptide Sll1443, preferably represented by SEQ ID NO. 53879, or a homolog or fragment thereof, or if the activity of a polypeptide encoded by a nucleic acid molecule comprising the nucleic acid SEQ ID NO. 53878, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 53878 or polypeptide SEQ ID NO. 53879, respectively, or a homolog or a fragment thereof, is increased or generated, or if the activity CTP synthetase is increased or generated in a non-human organism, like a microorganism or a plant cell, plant or part thereof, especially with mitochondrial localization, whereby the respective line discloses in column 7 aspartate. For example, an increase of the aspartate of at least 1 percent, particularly in a range of 58 to 119-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a nitrate/nitrite transport protein, or if the activity of the polypeptide Sll1450, preferably represented by SEQ ID NO. 54338, or a homolog or fragment thereof, or if the activity of a polypeptide encoded by a nucleic acid molecule comprising the nucleic acid SEQ ID NO. 54337, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 54337 or polypeptide SEQ ID NO. 54338, respectively, or a homolog or a fragment thereof, is increased or generated, or if the activity nitrate/nitrite transport protein is increased or generated in a non-human organism, like a microorganism or a plant cell, plant or part thereof, especially with mitochondrial localization, whereby the respective line discloses in column 7 glycine. For example, an increase of the glycine of at least 1 percent, particularly in a range of 36 to 102-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a nitrate/nitrite transport protein, or if the activity of the polypeptide Sll1450, preferably represented by SEQ ID NO. 54338, or a homolog or fragment thereof, or if the activity of a polypeptide encoded by a nucleic acid molecule comprising the nucleic acid SEQ ID NO. 54337, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 54337 or polypeptide SEQ ID NO. 54338, respectively, or a homolog or a fragment thereof, is increased or generated, or if the activity nitrate/nitrite transport protein is increased or generated in a non-human organism, like a microorganism or a plant cell, plant or part thereof, especially with mitochondrial localization, whereby the respective line discloses in column 7 phenylalanine. For example, an increase of the phenylalanine of at least 1 percent, particularly in a range of 32 to 42-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a nitrate/nitrite transport protein, or if the activity of the polypeptide Sll1450, preferably represented by SEQ ID NO. 54338, or a homolog or fragment thereof, or if the activity of a polypeptide encoded by a nucleic acid molecule comprising the nucleic acid SEQ ID NO. 54337, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 54337 or polypeptide SEQ ID NO. 54338, respectively, or a homolog or a fragment thereof, is increased or generated, or if the activity nitrate/nitrite transport protein is increased or generated in a non-human organism, like a microorganism or a plant cell, plant or part thereof, especially with mitochondrial localization, whereby the respective line discloses in column 7 serine. For example, an increase of the serine of at least 1 percent, particularly in a range of 24 to 83-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a nitrate/nitrite transport protein, or if the activity of the polypeptide Sll1450, preferably represented by SEQ ID NO. 54338, or a homolog or fragment thereof, or if the activity of a polypeptide encoded by a nucleic acid molecule comprising the nucleic acid SEQ ID NO. 54337, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 54337 or polypeptide SEQ ID NO. 54338, respectively, or a homolog or a fragment thereof, is increased or generated, or if the activity nitrate/nitrite transport protein is increased or generated in a non-human organism, like a microorganism or a plant cell, plant or part thereof, especially with mitochondrial localization, whereby the respective line discloses in column 7 tyrosine. For example, an increase of the tyrosine of at least 1 percent, particularly in a range of 37 to 92-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a CDP-diacylglycerol-glycerol-3-phosphate 3-phosphatidyltransferase, or if the activity of the polypeptide Sll1522, preferably represented by SEQ ID NO. 54453, or a homolog or fragment thereof, or if the activity of a polypeptide encoded by a nucleic acid molecule comprising the nucleic acid SEQ ID NO. 54452, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 54452 or polypeptide SEQ ID NO. 54453, respectively, or a homolog or a fragment thereof, is increased or generated, or if the activity CDP-diacylglycerol-glycerol-3-phosphate 3-phosphatidyltransferase is increased or generated in a non-human organism, like a microorganism or a plant cell, plant or part thereof, especially with plastidic localization, whereby the respective line discloses in column 7 aspartate. For example, an increase of the aspartate of at least 1 percent, particularly in a range of 50 to 230-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a CDP-diacylglycerol-glycerol-3-phosphate 3-phosphatidyltransferase, or if the activity of the polypeptide Sll1522, preferably represented by SEQ ID NO. 54453, or a homolog or fragment thereof, or if the activity of a polypeptide encoded by a nucleic acid molecule comprising the nucleic acid SEQ ID NO. 54452, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 54452 or polypeptide SEQ ID NO. 54453, respectively, or a homolog or a fragment thereof, is increased or generated, or if the activity CDP-diacylglycerol-glycerol-3-phosphate 3-phosphatidyltransferase is increased or generated in a non-human organism, like a microorganism or a plant cell, plant or part thereof, especially with plastidic localization, whereby the respective line discloses in column 7 5-oxoproline. For example, an increase of the 5-oxoproline of at least 1 percent, particularly in a range of 33 to 103-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a glutathione S-transferase, or if the activity of the polypeptide Sll1545, preferably represented by SEQ ID NO. 11424, or a homolog or fragment thereof, or if the activity of a polypeptide encoded by a nucleic acid molecule comprising the nucleic acid SEQ ID NO. 11423, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 11423 or polypeptide SEQ ID NO. 11424, respectively, or a homolog or a fragment thereof, is increased or generated, or if the activity glutathione S-transferase is increased or generated in a non-human organism, like a microorganism or a plant cell, plant or part thereof, especially with mitochondrial localization, whereby the respective line discloses in column 7 serine. For example, an increase of the serine of at least 1 percent, particularly in a range of 26 to 50-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a exopolyphosphatase, or if the activity of the polypeptide Sll1546, preferably represented by SEQ ID NO. 54805, or a homolog or fragment thereof, or if the activity of a polypeptide encoded by a nucleic acid molecule comprising the nucleic acid SEQ ID NO. 54804, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 54804 or polypeptide SEQ ID NO. 54805, respectively, or a homolog or a fragment thereof, is increased or generated, or if the activity exopolyphosphatase is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 51 to 102-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a exopolyphosphatase, or if the activity of the polypeptide Sll1546, preferably represented by SEQ ID NO. 54805, or a homolog or fragment thereof, or if the activity of a polypeptide encoded by a nucleic acid molecule comprising the nucleic acid SEQ ID NO. 54804, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 54804 or polypeptide SEQ ID NO. 54805, respectively, or a homolog or a fragment thereof, is increased or generated, or if the activity exopolyphosphatase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 27 to 53-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a exopolyphosphatase, or if the activity of the polypeptide Sll1546, preferably represented by SEQ ID NO. 54805, or a homolog or fragment thereof, or if the activity of a polypeptide encoded by a nucleic acid molecule comprising the nucleic acid SEQ ID NO. 54804, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 54804 or polypeptide SEQ ID NO. 54805, respectively, or a homolog or a fragment thereof, is increased or generated, or if the activity exopolyphosphatase is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 25 to 45-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a ABC metal ion transporter substrate-binding protein, or if the activity of the polypeptide Sll1598, preferably represented by SEQ ID NO. 85744, or a homolog or fragment thereof, or if the activity of a polypeptide encoded by a nucleic acid molecule comprising the nucleic acid SEQ ID NO. 85743, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 85743 or polypeptide SEQ ID NO. 85744, respectively, or a homolog or a fragment thereof, is increased or generated, or if the activity ABC metal ion transporter substrate-binding protein is increased or generated in a non-human organism, like a microorganism or a plant cell, plant or part thereof, especially with mitochondrial localization, whereby the respective line discloses in column 7 aspartate. For example, an increase of the aspartate of at least 1 percent, particularly in a range of 55 to 138-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a ABC metal ion transporter substrate-binding protein, or if the activity of the polypeptide Sll1598, preferably represented by SEQ ID NO. 85744, or a homolog or fragment thereof, or if the activity of a polypeptide encoded by a nucleic acid molecule comprising the nucleic acid SEQ ID NO. 85743, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 85743 or polypeptide SEQ ID NO. 85744, respectively, or a homolog or a fragment thereof, is increased or generated, or if the activity ABC metal ion transporter substrate-binding protein is increased or generated in a non-human organism, like a microorganism or a plant cell, plant or part thereof, especially with mitochondrial localization, whereby the respective line discloses in column 7 phenylalanine. For example, an increase of the phenylalanine of at least 1 percent, particularly in a range of 26 to 113-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a sll1761-protein, or if the activity of the polypeptide Sll1761, preferably represented by SEQ ID NO. 55380, or a homolog or fragment thereof, or if the activity of a polypeptide encoded by a nucleic acid molecule comprising the nucleic acid SEQ ID NO. 55379, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 55379 or polypeptide SEQ ID NO. 55380, respectively, or a homolog or a fragment thereof, is increased or generated, or if the activity sll1761-protein is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 23 to 63-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a sll1761-protein, or if the activity of the polypeptide Sll1761, preferably represented by SEQ ID NO. 55380, or a homolog or fragment thereof, or if the activity of a polypeptide encoded by a nucleic acid molecule comprising the nucleic acid SEQ ID NO. 55379, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 55379 or polypeptide SEQ ID NO. 55380, respectively, or a homolog or a fragment thereof, is increased or generated, or if the activity sll1761-protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 39 to 141-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a adenylate kinase, or if the activity of the polypeptide Sll1815, preferably represented by SEQ ID NO. 85809, or a homolog or fragment thereof, or if the activity of a polypeptide encoded by a nucleic acid molecule comprising the nucleic acid SEQ ID NO. 85808, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 85808 or polypeptide SEQ ID NO. 85809, respectively, or a homolog or a fragment thereof, is increased or generated, or if the activity adenylate kinase is increased or generated in a non-human organism, like a microorganism or a plant cell, plant or part thereof, especially with mitochondrial localization, whereby the respective line discloses in column 7 alanine. For example, an increase of the alanine of at least 1 percent, particularly in a range of 31 to 483-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a adenylate kinase, or if the activity of the polypeptide Sll1815, preferably represented by SEQ ID NO. 85809, or a homolog or fragment thereof, or if the activity of a polypeptide encoded by a nucleic acid molecule comprising the nucleic acid SEQ ID NO. 85808, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 85808 or polypeptide SEQ ID NO. 85809, respectively, or a homolog or a fragment thereof, is increased or generated, or if the activity adenylate kinase is increased or generated in a non-human organism, like a microorganism or a plant cell, plant or part thereof, especially with mitochondrial localization, whereby the respective line discloses in column 7 citrulline. For example, an increase of the citrulline of at least 1 percent, particularly in a range of 39 to 122-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a adenylate kinase, or if the activity of the polypeptide Sll1815, preferably represented by SEQ ID NO. 85809, or a homolog or fragment thereof, or if the activity of a polypeptide encoded by a nucleic acid molecule comprising the nucleic acid SEQ ID NO. 85808, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 85808 or polypeptide SEQ ID NO. 85809, respectively, or a homolog or a fragment thereof, is increased or generated, or if the activity adenylate kinase is increased or generated in a non-human organism, like a microorganism or a plant cell, plant or part thereof, especially with mitochondrial localization, whereby the respective line discloses in column 7 glycine. For example, an increase of the glycine of at least 1 percent, particularly in a range of 118 to 238-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a adenylate kinase, or if the activity of the polypeptide Sll1815, preferably represented by SEQ ID NO. 85809, or a homolog or fragment thereof, or if the activity of a polypeptide encoded by a nucleic acid molecule comprising the nucleic acid SEQ ID NO. 85808, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 85808 or polypeptide SEQ ID NO. 85809, respectively, or a homolog or a fragment thereof, is increased or generated, or if the activity adenylate kinase is increased or generated in a non-human organism, like a microorganism or a plant cell, plant or part thereof, especially with mitochondrial localization, whereby the respective line discloses in column 7 serine. For example, an increase of the serine of at least 1 percent, particularly in a range of 55 to 83-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a adenylate kinase, or if the activity of the polypeptide Sll1815, preferably represented by SEQ ID NO. 85809, or a homolog or fragment thereof, or if the activity of a polypeptide encoded by a nucleic acid molecule comprising the nucleic acid SEQ ID NO. 85808, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 85808 or polypeptide SEQ ID NO. 85809, respectively, or a homolog or a fragment thereof, is increased or generated, or if the activity adenylate kinase is increased or generated in a non-human organism, like a microorganism or a plant cell, plant or part thereof, especially with mitochondrial localization, whereby the respective line discloses in column 7 tyrosine. For example, an increase of the tyrosine of at least 1 percent, particularly in a range of 39 to 88-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a acyl transferase, or if the activity of the polypeptide Sll1848, preferably represented by SEQ ID NO. 86448, or a homolog or fragment thereof, or if the activity of a polypeptide encoded by a nucleic acid molecule comprising the nucleic acid SEQ ID NO. 86447, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 86447 or polypeptide SEQ ID NO. 86448, respectively, or a homolog or a fragment thereof, is increased or generated, or if the activity acyl transferase is increased or generated in a non-human organism, like a microorganism or a plant cell, plant or part thereof, especially with plastidic localization, whereby the respective line discloses in column 7 glycine. For example, an increase of the glycine of at least 1 percent, particularly in a range of 48 to 205-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a acyl transferase, or if the activity of the polypeptide Sll1848, preferably represented by SEQ ID NO. 86448, or a homolog or fragment thereof, or if the activity of a polypeptide encoded by a nucleic acid molecule comprising the nucleic acid SEQ ID NO. 86447, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 86447 or polypeptide SEQ ID NO. 86448, respectively, or a homolog or a fragment thereof, is increased or generated, or if the activity acyl transferase is increased or generated in a non-human organism, like a microorganism or a plant cell, plant or part thereof, especially with plastidic localization, whereby the respective line discloses in column 7 phenylalanine. For example, an increase of the phenylalanine of at least 1 percent, particularly in a range of 26 to 318-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a acyl transferase, or if the activity of the polypeptide Sll1848, preferably represented by SEQ ID NO. 86448, or a homolog or fragment thereof, or if the activity of a polypeptide encoded by a nucleic acid molecule comprising the nucleic acid SEQ ID NO. 86447, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 86447 or polypeptide SEQ ID NO. 86448, respectively, or a homolog or a fragment thereof, is increased or generated, or if the activity acyl transferase is increased or generated in a non-human organism, like a microorganism or a plant cell, plant or part thereof, especially with plastidic localization, whereby the respective line discloses in column 7 serine. For example, an increase of the serine of at least 1 percent, particularly in a range of 28 to 319-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a coproporphyrinogen III oxidase, or if the activity of the polypeptide Sll1917, preferably represented by SEQ ID NO. 11472, or a homolog or fragment thereof, or if the activity of a polypeptide encoded by a nucleic acid molecule comprising the nucleic acid SEQ ID NO. 11471, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 11471 or polypeptide SEQ ID NO. 11472, respectively, or a homolog or a fragment thereof, is increased or generated, or if the activity coproporphyrinogen III oxidase is increased or generated in a non-human organism, like a microorganism or a plant cell, plant or part thereof, especially with mitochondrial localization, whereby the respective line discloses in column 7 aspartate. For example, an increase of the aspartate of at least 1 percent, particularly in a range of 64 to 265-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a coproporphyrinogen III oxidase, or if the activity of the polypeptide Sll1917, preferably represented by SEQ ID NO. 11472, or a homolog or fragment thereof, or if the activity of a polypeptide encoded by a nucleic acid molecule comprising the nucleic acid SEQ ID NO. 11471, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 11471 or polypeptide SEQ ID NO. 11472, respectively, or a homolog or a fragment thereof, is increased or generated, or if the activity coproporphyrinogen III oxidase is increased or generated in a non-human organism, like a microorganism or a plant cell, plant or part thereof, especially with mitochondrial localization, whereby the respective line discloses in column 7 citrulline. For example, an increase of the citrulline of at least 1 percent, particularly in a range of 41 to 71-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a coproporphyrinogen III oxidase, or if the activity of the polypeptide Sll1917, preferably represented by SEQ ID NO. 11472, or a homolog or fragment thereof, or if the activity of a polypeptide encoded by a nucleic acid molecule comprising the nucleic acid SEQ ID NO. 11471, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 11471 or polypeptide SEQ ID NO. 11472, respectively, or a homolog or a fragment thereof, is increased or generated, or if the activity coproporphyrinogen Ill oxidase is increased or generated in a non-human organism, like a microorganism or a plant cell, plant or part thereof, especially with mitochondrial localization, whereby the respective line discloses in column 7 phenylalanine. For example, an increase of the phenylalanine of at least 1 percent, particularly in a range of 32 to 56 percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a coproporphyrinogen III oxidase, or if the activity of the polypeptide Sll1917, preferably represented by SEQ ID NO. 11472, or a homolog or fragment thereof, or if the activity of a polypeptide encoded by a nucleic acid molecule comprising the nucleic acid SEQ ID NO. 11471, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 11471 or polypeptide SEQ ID NO. 11472, respectively, or a homolog or a fragment thereof, is increased or generated, or if the activity coproporphyrinogen III oxidase is increased or generated in a non-human organism, like a microorganism or a plant cell, plant or part thereof, especially with mitochondrial localization, whereby the respective line discloses in column 7 serine. For example, an increase of the serine of at least 1 percent, particularly in a range of 28 to 69-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a coproporphyrinogen III oxidase, or if the activity of the polypeptide Sll1917, preferably represented by SEQ ID NO. 11472, or a homolog or fragment thereof, or if the activity of a polypeptide encoded by a nucleic acid molecule comprising the nucleic acid SEQ ID NO. 11471, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 11471 or polypeptide SEQ ID NO. 11472, respectively, or a homolog or a fragment thereof, is increased or generated, or if the activity coproporphyrinogen III oxidase is increased or generated in a non-human organism, like a microorganism or a plant cell, plant or part thereof, especially with mitochondrial localization, whereby the respective line discloses in column 7 tyrosine. For example, an increase of the tyrosine of at least 1 percent, particularly in a range of 35 to 60-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a cation-transporting ATPase, or if the activity of the polypeptide Sll1920, preferably represented by SEQ ID NO. 55386, or a homolog or fragment thereof, or if the activity of a polypeptide encoded by a nucleic acid molecule comprising the nucleic acid SEQ ID NO. 55385, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 55385 or polypeptide SEQ ID NO. 55386, respectively, or a homolog or a fragment thereof, is increased or generated, or if the activity cation-transporting ATPase is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 52 to 204-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a cation-transporting ATPase, or if the activity of the polypeptide Sll1920, preferably represented by SEQ ID NO. 55386, or a homolog or fragment thereof, or if the activity of a polypeptide encoded by a nucleic acid molecule comprising the nucleic acid SEQ ID NO. 55385, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 55385 or polypeptide SEQ ID NO. 55386, respectively, or a homolog or a fragment thereof, is increased or generated, or if the activity cation-transporting ATPase is increased or generated in a non-human organism, like a microorganism or a plant cell, plant or part thereof, especially with mitochondrial localization, whereby the respective line discloses in column 7 aspartate. For example, an increase of the aspartate of at least 1 percent, particularly in a range of 65 to 142-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a glycosyl transferase, or if the activity of the polypeptide Sll2003, preferably represented by SEQ ID NO. 86509, or a homolog or fragment thereof, or if the activity of a polypeptide encoded by a nucleic acid molecule comprising the nucleic acid SEQ ID NO. 86508, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 86508 or polypeptide SEQ ID NO. 86509, respectively, or a homolog or a fragment thereof, is increased or generated, or if the activity glycosyl transferase is increased or generated in a non-human organism, like a microorganism or a plant cell, plant or part thereof, especially with plastidic localization, whereby the respective line discloses in column 7 glycine. For example, an increase of the glycine of at least 1 percent, particularly in a range of 47 to 67-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a precorrin methylase, or if the activity of the polypeptide Slr0239, preferably represented by SEQ ID NO. 86541, or a homolog or fragment thereof, or if the activity of a polypeptide encoded by a nucleic acid molecule comprising the nucleic acid SEQ ID NO. 86540, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 86540 or polypeptide SEQ ID NO. 86541, respectively, or a homolog or a fragment thereof, is increased or generated, or if the activity precorrin methylase is increased or generated in a non-human organism, like a microorganism or a plant cell, plant or part thereof, especially with mitochondrial localization, whereby the respective line discloses in column 7 phenylalanine. For example, an increase of the phenylalanine of at least 1 percent, particularly in a range of 41 to 279-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a precorrin methylase, or if the activity of the polypeptide Slr0239, preferably represented by SEQ ID NO. 86541, or a homolog or fragment thereof, or if the activity of a polypeptide encoded by a nucleic acid molecule comprising the nucleic acid SEQ ID NO. 86540, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 86540 or polypeptide SEQ ID NO. 86541, respectively, or a homolog or a fragment thereof, is increased or generated, or if the activity precorrin methylase is increased or generated in a non-human organism, like a microorganism or a plant cell, plant or part thereof, especially with mitochondrial localization, whereby the respective line discloses in column 7 tyrosine. For example, an increase of the tyrosine of at least 1 percent, particularly in a range of 54 to 214-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a aldehyde dehydrogenase, or if the activity of the polypeptide Slr0370, preferably represented by SEQ ID NO. 86821, or a homolog or fragment thereof, or if the activity of a polypeptide encoded by a nucleic acid molecule comprising the nucleic acid SEQ ID NO. 86820, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 86820 or polypeptide SEQ ID NO. 86821, respectively, or a homolog or a fragment thereof, is increased or generated, or if the activity aldehyde dehydrogenase is increased or generated in a non-human organism, like a microorganism or a plant cell, plant or part thereof, especially with plastidic localization, whereby the respective line discloses in column 7 citrulline. For example, an increase of the citrulline of at least 1 percent, particularly in a range of 38 to 90-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a bifunctional purine biosynthesis protein, or if the activity of the polypeptide Slr0597, preferably represented by SEQ ID NO. 56154, or a homolog or fragment thereof, or if the activity of a polypeptide encoded by a nucleic acid molecule comprising the nucleic acid SEQ ID NO. 56153, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 56153 or polypeptide SEQ ID NO. 56154, respectively, or a homolog or a fragment thereof, is increased or generated, or if the activity bifunctional purine biosynthesis protein is increased or generated in a non-human organism, like a microorganism or a plant cell, plant or part thereof, especially with plastidic localization, whereby the respective line discloses in column 7 alanine. For example, an increase of the alanine of at least 1 percent, particularly in a range of 28 to 38-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a bifunctional purine biosynthesis protein, or if the activity of the polypeptide Slr0597, preferably represented by SEQ ID NO. 56154, or a homolog or fragment thereof, or if the activity of a polypeptide encoded by a nucleic acid molecule comprising the nucleic acid SEQ ID NO. 56153, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 56153 or polypeptide SEQ ID NO. 56154, respectively, or a homolog or a fragment thereof, is increased or generated, or if the activity bifunctional purine biosynthesis protein is increased or generated in a non-human organism, like a microorganism or a plant cell, plant or part thereof, especially with plastidic localization, whereby the respective line discloses in column 7 serine. For example, an increase of the serine of at least 1 percent, particularly in a range of 27 to 104-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a bifunctional purine biosynthesis protein, or if the activity of the polypeptide Slr0597, preferably represented by SEQ ID NO. 56154, or a homolog or fragment thereof, or if the activity of a polypeptide encoded by a nucleic acid molecule comprising the nucleic acid SEQ ID NO. 56153, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 56153 or polypeptide SEQ ID NO. 56154, respectively, or a homolog or a fragment thereof, is increased or generated, or if the activity bifunctional purine biosynthesis protein is increased or generated in a non-human organism, like a microorganism or a plant cell, plant or part thereof, especially with plastidic localization, whereby the respective line discloses in column 7 5-oxoproline. For example, an increase of the 5-oxoproline of at least 1 percent, particularly in a range of 40 to 96 percent is conferred as compared to a corresponding non-transformed wild type non-human organism.

Accordingly, in one embodiment, an increase of asparagine in a non-human organism, as compared to a corresponding non-transformed wild type non-human organism, is conferred in the process of the invention, if the activity of a polypeptide showing the activity of a Glu/Leu/Phe/Val dehydrogenase , or if the activity of the polypeptide Slr0710, preferably represented by SEQ ID NO. 56895, or a homolog or fragment thereof, or if the activity of a polypeptide encoded by a nucleic acid molecule comprising the nucleic acid SEQ ID NO. 56894, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 56894 or polypeptide SEQ ID NO. 56895, respectively, or a homolog or a fragment thereof, is increased or generated, or if the activity Glu/Leu/Phe/Val dehydrogenase is increased or generated in a non-human organism, like a microorganism or a plant cell, plant or part thereof, especially with mitochondrial localization, whereby the respective line discloses in column 7 asparagine. For example, an increase of the asparagine of at least 1 percent, particularly in a range of 49 to 215-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a Glu/Leu/Phe/Val dehydrogenase , or if the activity of the polypeptide Slr0710, preferably represented by SEQ ID NO. 56895, or a homolog or fragment thereof, or if the activity of a polypeptide encoded by a nucleic acid molecule comprising the nucleic acid SEQ ID NO. 56894, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 56894 or polypeptide SEQ ID NO. 56895, respectively, or a homolog or a fragment thereof, is increased or generated, or if the activity Glu/Leu/Phe/Val dehydrogenase is increased or generated in a non-human organism, like a microorganism or a plant cell, plant or part thereof, especially with mitochondrial localization, whereby the respective line discloses in column 7 phenylalanine. For example, an increase of the phenylalanine of at least 1 percent, particularly in a range of 26 to 99-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a Glu/Leu/Phe/Val dehydrogenase , or if the activity of the polypeptide Slr0710, preferably represented by SEQ ID NO. 56895, or a homolog or fragment thereof, or if the activity of a polypeptide encoded by a nucleic acid molecule comprising the nucleic acid SEQ ID NO. 56894, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 56894 or polypeptide SEQ ID NO. 56895, respectively, or a homolog or a fragment thereof, is increased or generated, or if the activity Glu/Leu/Phe/Val dehydrogenase is increased or generated in a non-human organism, like a microorganism or a plant cell, plant or part thereof, especially with mitochondrial localization, whereby the respective line discloses in column 7 tyrosine. For example, an increase of the tyrosine of at least 1 percent, particularly in a range of 37 to 80-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a malate dehydrogenase, or if the activity of the polypeptide Slr0721, preferably represented by SEQ ID NO. 87398, or a homolog or fragment thereof, or if the activity of a polypeptide encoded by a nucleic acid molecule comprising the nucleic acid SEQ ID NO. 87397, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 87397 or polypeptide SEQ ID NO. 87398, respectively, or a homolog or a fragment thereof, is increased or generated, or if the activity malate dehydrogenase is increased or generated in a non-human organism, like a microorganism or a plant cell, plant or part thereof, especially with plastidic localization, whereby the respective line discloses in column 7 phenylalanine. For example, an increase of the phenylalanine of at least 1 percent, particularly in a range of 30 to 107-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a geranylgeranyl pyrophosphate synthase, or if the activity of the polypeptide Slr0739, preferably represented by SEQ ID NO. 57236, or a homolog or fragment thereof, or if the activity of a polypeptide encoded by a nucleic acid molecule comprising the nucleic acid SEQ ID NO. 57235, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 57235 or polypeptide SEQ ID NO. 57236, respectively, or a homolog or a fragment thereof, is increased or generated, or if the activity geranylgeranyl pyrophosphate synthase is increased or generated in a non-human organism, like a microorganism or a plant cell, plant or part thereof, especially with plastidic localization, whereby the respective line discloses in column 7 alanine. For example, an increase of the alanine of at least 1 percent, particularly in a range of 19 to 56-percent is conferred as compared to a corresponding non-transformed wild type non-human organism.

Accordingly, in one embodiment, an increase of asparagine in a non-human organism, as compared to a corresponding non-transformed wild type non-human organism, is conferred in the process of the invention, if the activity of a polypeptide showing the activity of a geranylgeranyl pyrophosphate synthase, or if the activity of the polypeptide Slr0739, preferably represented by SEQ ID NO. 57236, or a homolog or fragment thereof, or if the activity of a polypeptide encoded by a nucleic acid molecule comprising the nucleic acid SEQ ID NO. 57235, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 57235 or polypeptide SEQ ID NO. 57236, respectively, or a homolog or a fragment thereof, is increased or generated, or if the activity geranylgeranyl pyrophosphate synthase is increased or generated in a non-human organism, like a microorganism or a plant cell, plant or part thereof, especially with plastidic localization, whereby the respective line discloses in column 7 asparagine. For example, an increase of the asparagine of at least 1 percent, particularly in a range of 155 to 1305-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a geranylgeranyl pyrophosphate synthase, or if the activity of the polypeptide Slr0739, preferably represented by SEQ ID NO. 57236, or a homolog or fragment thereof, or if the activity of a polypeptide encoded by a nucleic acid molecule comprising the nucleic acid SEQ ID NO. 57235, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 57235 or polypeptide SEQ ID NO. 57236, respectively, or a homolog or a fragment thereof, is increased or generated, or if the activity geranylgeranyl pyrophosphate synthase is increased or generated in a non-human organism, like a microorganism or a plant cell, plant or part thereof, especially with plastidic localization, whereby the respective line discloses in column 7 phenylalanine. For example, an increase of the phenylalanine of at least 1 percent, particularly in a range of 44 to 71-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a geranylgeranyl pyrophosphate synthase, or if the activity of the polypeptide Slr0739, preferably represented by SEQ ID NO. 57236, or a homolog or fragment thereof, or if the activity of a polypeptide encoded by a nucleic acid molecule comprising the nucleic acid SEQ ID NO. 57235, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 57235 or polypeptide SEQ ID NO. 57236, respectively, or a homolog or a fragment thereof, is increased or generated, or if the activity geranylgeranyl pyrophosphate synthase is increased or generated in a non-human organism, like a microorganism or a plant cell, plant or part thereof, especially with plastidic localization, whereby the respective line discloses in column 7 serine. For example, an increase of the serine of at least 1 percent, particularly in a range of 52 to 166-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a geranylgeranyl pyrophosphate synthase, or if the activity of the polypeptide Slr0739, preferably represented by SEQ ID NO. 57236, or a homolog or fragment thereof, or if the activity of a polypeptide encoded by a nucleic acid molecule comprising the nucleic acid SEQ ID NO. 57235, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 57235 or polypeptide SEQ ID NO. 57236, respectively, or a homolog or a fragment thereof, is increased or generated, or if the activity geranylgeranyl pyrophosphate synthase is increased or generated in a non-human organism, like a microorganism or a plant cell, plant or part thereof, especially with plastidic localization, whereby the respective line discloses in column 7 tyrosine. For example, an increase of the tyrosine of at least 1 percent, particularly in a range of 66 to 82-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a geranylgeranyl pyrophosphate synthase, or if the activity of the polypeptide Slr0739, preferably represented by SEQ ID NO. 57236, or a homolog or fragment thereof, or if the activity of a polypeptide encoded by a nucleic acid molecule comprising the nucleic acid SEQ ID NO. 57235, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 57235 or polypeptide SEQ ID NO. 57236, respectively, or a homolog or a fragment thereof, is increased or generated, or if the activity geranylgeranyl pyrophosphate synthase is increased or generated in a non-human organism, like a microorganism or a plant cell, plant or part thereof, especially with plastidic localization, whereby the respective line discloses in column 7 5-oxoproline. For example, an increase of the 5-oxoproline of at least 1 percent, particularly in a range of 33 to 130-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a amine oxidase, or if the activity of the polypeptide Slr0782, preferably represented by SEQ ID NO. 57680, or a homolog or fragment thereof, or if the activity of a polypeptide encoded by a nucleic acid molecule comprising the nucleic acid SEQ ID NO. 57679, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 57679 or polypeptide SEQ ID NO. 57680, respectively, or a homolog or a fragment thereof, is increased or generated, or if the activity amine oxidase is increased or generated in a non-human organism, like a microorganism or a plant cell, plant or part thereof, especially with mitochondrial localization, whereby the respective line discloses in column 7 phenylalanine. For example, an increase of the phenylalanine of at least 1 percent, particularly in a range of 34 to 38-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a kinase, or if the activity of the polypeptide Slr0862, preferably represented by SEQ ID NO. 87611, or a homolog or fragment thereof, or if the activity of a polypeptide encoded by a nucleic acid molecule comprising the nucleic acid SEQ ID NO. 87610, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 87610 or polypeptide SEQ ID NO. 87611, respectively, or a homolog or a fragment thereof, is increased or generated, or if the activity kinase is increased or generated in a non-human organism, like a microorganism or a plant cell, plant or part thereof, especially with mitochondrial localization, whereby the respective line discloses in column 7 serine. For example, an increase of the serine of at least 1 percent, particularly in a range of 23 to 45-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a tryptophan synthase alpha chain, or if the activity of the polypeptide Slr0966, preferably represented by SEQ ID NO. 87656, or a homolog or fragment thereof, or if the activity of a polypeptide encoded by a nucleic acid molecule comprising the nucleic acid SEQ ID NO. 87655, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 87655 or polypeptide SEQ ID NO. 87656, respectively, or a homolog or a fragment thereof, is increased or generated, or if the activity tryptophan synthase alpha chain is increased or generated in a non-human organism, like a microorganism or a plant cell, plant or part thereof, especially with plastidic localization, whereby the respective line discloses in column 7 tyrosine. For example, an increase of the tyrosine of at least 1 percent, particularly in a range of 43 to 77-percent is conferred as compared to a corresponding non-transformed wild type non-human organism.

Accordingly, in one embodiment, an increase of ornithine in a non-human organism, as compared to a corresponding non-transformed wild type non-human organism, is conferred in the process of the invention, if the activity of a polypeptide showing the activity of a argininosuccinate lyase, or if the activity of the polypeptide Slr1133, preferably represented by SEQ ID NO. 87854, or a homolog or fragment thereof, or if the activity of a polypeptide encoded by a nucleic acid molecule comprising the nucleic acid SEQ ID NO. 87853, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 87853 or polypeptide SEQ ID NO. 87854, respectively, or a homolog or a fragment thereof, is increased or generated, or if the activity argininosuccinate lyase is increased or generated in a non-human organism, like a microorganism or a plant cell, plant or part thereof, especially with mitochondrial localization, whereby the respective line discloses in column 7 ornithine. For example, an increase of the ornithine of at least 1 percent, particularly in a range of 69 to 390-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a argininosuccinate lyase, or if the activity of the polypeptide Slr1133, preferably represented by SEQ ID NO. 87854, or a homolog or fragment thereof, or if the activity of a polypeptide encoded by a nucleic acid molecule comprising the nucleic acid SEQ ID NO. 87853, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 87853 or polypeptide SEQ ID NO. 87854, respectively, or a homolog or a fragment thereof, is increased or generated, or if the activity argininosuccinate lyase is increased or generated in a non-human organism, like a microorganism or a plant cell, plant or part thereof, especially with mitochondrial localization, whereby the respective line discloses in column 7 serine. For example, an increase of the serine of at least 1 percent, particularly in a range of 26 to 69-percent is conferred as compared to a corresponding non-transformed wild type non-human organism.

Accordingly, in one embodiment, an increase of asparagine in a non-human organism, as compared to a corresponding non-transformed wild type non-human organism, is conferred in the process of the invention, if the activity of a polypeptide showing the activity of a gammaglutamyltranspeptidase, or if the activity of the polypeptide Slr1269, preferably represented by SEQ ID NO. 58059, or a homolog or fragment thereof, or if the activity of a polypeptide encoded by a nucleic acid molecule comprising the nucleic acid SEQ ID NO. 58058, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 58058 or polypeptide SEQ ID NO. 58059, respectively, or a homolog or a fragment thereof, is increased or generated, or if the activity gamma-glutamyltranspeptidase is increased or generated in a non-human organism, like a microorganism or a plant cell, plant or part thereof, especially with non-targeted localization, whereby the respective line discloses in column 7 asparagine. For example, an increase of the asparagine of at least 1 percent, particularly in a range of 45 to 93-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a iron(III) dicitrate-binding protein, or if the activity of the polypeptide Slr1492, preferably represented by SEQ ID NO. 58669, or a homolog or fragment thereof, or if the activity of a polypeptide encoded by a nucleic acid molecule comprising the nucleic acid SEQ ID NO. 58668, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 58668 or polypeptide SEQ ID NO. 58669, respectively, or a homolog or a fragment thereof, is increased or generated, or if the activity iron(III) dicitrate-binding protein is increased or generated in a non-human organism, like a microorganism or a plant cell, plant or part thereof, especially with mitochondrial localization, whereby the respective line discloses in column 7 aspartate. For example, an increase of the aspartate of at least 1 percent, particularly in a range of 48 to 139-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a iron(III) dicitrate-binding protein, or if the activity of the polypeptide Slr1492, preferably represented by SEQ ID NO. 58669, or a homolog or fragment thereof, or if the activity of a polypeptide encoded by a nucleic acid molecule comprising the nucleic acid SEQ ID NO. 58668, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 58668 or polypeptide SEQ ID NO. 58669, respectively, or a homolog or a fragment thereof, is increased or generated, or if the activity iron(III) dicitrate-binding protein is increased or generated in a non-human organism, like a microorganism or a plant cell, plant or part thereof, especially with mitochondrial localization, whereby the respective line discloses in column 7 phenylalanine. For example, an increase of the phenylalanine of at least 1 percent, particularly in a range of 31 to 35-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a Na+/K+ transporter, or if the activity of the polypeptide Slr1509, preferably represented by SEQ ID NO. 88159, or a homolog or fragment thereof, or if the activity of a polypeptide encoded by a nucleic acid molecule comprising the nucleic acid SEQ ID NO. 88158, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 88158 or polypeptide SEQ ID NO. 88159, respectively, or a homolog or a fragment thereof, is increased or generated, or if the activity Na+/K+ transporter is increased or generated in a non-human organism, like a microorganism or a plant cell, plant or part thereof, especially with mitochondrial localization, whereby the respective line discloses in column 7 phenylalanine. For example, an increase of the phenylalanine of at least 1 percent, particularly in a range of 43 to 94-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a Na+/K+ transporter, or if the activity of the polypeptide Slr1509, preferably represented by SEQ ID NO. 88159, or a homolog or fragment thereof, or if the activity of a polypeptide encoded by a nucleic acid molecule comprising the nucleic acid SEQ ID NO. 88158, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 88158 or polypeptide SEQ ID NO. 88159, respectively, or a homolog or a fragment thereof, is increased or generated, or if the activity Na+/K+ transporter is increased or generated in a non-human organism, like a microorganism or a plant cell, plant or part thereof, especially with mitochondrial localization, whereby the respective line discloses in column 7 tyrosine. For example, an increase of the tyrosine of at least 1 percent, particularly in a range of 67 to 86-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a 3-oxoacyl-[acyl-carrier-protein] synthase, or if the activity of the polypeptide Slr1511, preferably represented by SEQ ID NO. 88272, or a homolog or fragment thereof, or if the activity of a polypeptide encoded by a nucleic acid molecule comprising the nucleic acid SEQ ID NO. 88271, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 88271 or polypeptide SEQ ID NO. 88272, respectively, or a homolog or a fragment thereof, is increased or generated, or if the activity 3-oxoacyl-[acyl-carrier-protein] synthase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 30 to 31-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a 3-oxoacyl-[acyl-carrier-protein] synthase, or if the activity of the polypeptide Slr1511, preferably represented by SEQ ID NO. 88272, or a homolog or fragment thereof, or if the activity of a polypeptide encoded by a nucleic acid molecule comprising the nucleic acid SEQ ID NO. 88271, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 88271 or polypeptide SEQ ID NO. 88272, respectively, or a homolog or a fragment thereof, is increased or generated, or if the activity 3-oxoacyl-[acyl-carrier-protein] synthase is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 30 to 71-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a Photosystem I reaction center subunit XI, or if the activity of the polypeptide Slr1655, preferably represented by SEQ ID NO. 12071, or a homolog or fragment thereof, or if the activity of a polypeptide encoded by a nucleic acid molecule comprising the nucleic acid SEQ ID NO. 12070, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 12070 or polypeptide SEQ ID NO. 12071, respectively, or a homolog or a fragment thereof, is increased or generated, or if the activity Photosystem I reaction center subunit XI is increased or generated in a non-human organism, like a microorganism or a plant cell, plant or part thereof, especially with mitochondrial localization, whereby the respective line discloses in column 7 phenylalanine. For example, an increase of the phenylalanine of at least 1 percent, particularly in a range of 28 to 60-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a Photosystem I reaction center subunit XI, or if the activity of the polypeptide Slr1655, preferably represented by SEQ ID NO. 12071, or a homolog or fragment thereof, or if the activity of a polypeptide encoded by a nucleic acid molecule comprising the nucleic acid SEQ ID NO. 12070, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 12070 or polypeptide SEQ ID NO. 12071, respectively, or a homolog or a fragment thereof, is increased or generated, or if the activity Photosystem I reaction center subunit XI is increased or generated in a non-human organism, like a microorganism or a plant cell, plant or part thereof, especially with mitochondrial localization, whereby the respective line discloses in column 7 serine. For example, an increase of the serine of at least 1 percent, particularly in a range of 33 to 83-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a Photosystem I reaction center subunit XI, or if the activity of the polypeptide Slr1655, preferably represented by SEQ ID NO. 12071, or a homolog or fragment thereof, or if the activity of a polypeptide encoded by a nucleic acid molecule comprising the nucleic acid SEQ ID NO. 12070, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 12070 or polypeptide SEQ ID NO. 12071, respectively, or a homolog or a fragment thereof, is increased or generated, or if the activity Photosystem I reaction center subunit XI is increased or generated in a non-human organism, like a microorganism or a plant cell, plant or part thereof, especially with mitochondrial localization, whereby the respective line discloses in column 7 tyrosine. For example, an increase of the tyrosine of at least 1 percent, particularly in a range of 35 to 104-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a photosystem II protein, or if the activity of the polypeptide Slr1739, preferably represented by SEQ ID NO. 58732, or a homolog or fragment thereof, or if the activity of a polypeptide encoded by a nucleic acid molecule comprising the nucleic acid SEQ ID NO. 58731, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 58731 or polypeptide SEQ ID NO. 58732, respectively, or a homolog or a fragment thereof, is increased or generated, or if the activity photosystem II protein is increased or generated in a non-human organism, like a microorganism or a plant cell, plant or part thereof, especially with plastidic localization, whereby the respective line discloses in column 7 phenylalanine. For example, an increase of the phenylalanine of at least 1 percent, particularly in a range of 47 to 192-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a photosystem II protein, or if the activity of the polypeptide Slr1739, preferably represented by SEQ ID NO. 58732, or a homolog or fragment thereof, or if the activity of a polypeptide encoded by a nucleic acid molecule comprising the nucleic acid SEQ ID NO. 58731, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 58731 or polypeptide SEQ ID NO. 58732, respectively, or a homolog or a fragment thereof, is increased or generated, or if the activity photosystem II protein is increased or generated in a non-human organism, like a microorganism or a plant cell, plant or part thereof, especially with plastidic localization, whereby the respective line discloses in column 7 serine. For example, an increase of the serine of at least 1 percent, particularly in a range of 59 to 99-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a photosystem II protein, or if the activity of the polypeptide Slr1739, preferably represented by SEQ ID NO. 58732, or a homolog or fragment thereof, or if the activity of a polypeptide encoded by a nucleic acid molecule comprising the nucleic acid SEQ ID NO. 58731, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 58731 or polypeptide SEQ ID NO. 58732, respectively, or a homolog or a fragment thereof, is increased or generated, or if the activity photosystem II protein is increased or generated in a non-human organism, like a microorganism or a plant cell, plant or part thereof, especially with plastidic localization, whereby the respective line discloses in column 7 tyrosine. For example, an increase of the tyrosine of at least 1 percent, particularly in a range of 73 to 163-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a photosystem II protein, or if the activity of the polypeptide Slr1739, preferably represented by SEQ ID NO. 58732, or a homolog or fragment thereof, or if the activity of a polypeptide encoded by a nucleic acid molecule comprising the nucleic acid SEQ ID NO. 58731, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 58731 or polypeptide SEQ ID NO. 58732, respectively, or a homolog or a fragment thereof, is increased or generated, or if the activity photosystem II protein is increased or generated in a non-human organism, like a microorganism or a plant cell, plant or part thereof, especially with plastidic localization, whereby the respective line discloses in column 7 5-oxoproline. For example, an increase of the 5-oxoproline of at least 1 percent, particularly in a range of 46 to 128-percent is conferred as compared to a corresponding non-transformed wild type non-human organism.

Accordingly, in one embodiment, an increase of asparagine in a non-human organism, as compared to a corresponding non-transformed wild type non-human organism, is conferred in the process of the invention, if the activity of a polypeptide showing the activity of a glutamine amidotransferase, or if the activity of the polypeptide Slr1742, preferably represented by SEQ ID NO. 58752, or a homolog or fragment thereof, or if the activity of a polypeptide encoded by a nucleic acid molecule comprising the nucleic acid SEQ ID NO. 58751, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 58751 or polypeptide SEQ ID NO. 58752, respectively, or a homolog or a fragment thereof, is increased or generated, or if the activity glutamine amidotransferase is increased or generated in a non-human organism, like a microorganism or a plant cell, plant or part thereof, especially with mitochondrial localization, whereby the respective line discloses in column 7 asparagine. For example, an increase of the asparagine of at least 1 percent, particularly in a range of 45 to 188-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a phosphoadenosine phosphosulfate reductase , or if the activity of the polypeptide Slr1791, preferably represented by SEQ ID NO. 12141, or a homolog or fragment thereof, or if the activity of a polypeptide encoded by a nucleic acid molecule comprising the nucleic acid SEQ ID NO. 12140, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 12140 or polypeptide SEQ ID NO. 12141, respectively, or a homolog or a fragment thereof, is increased or generated, or if the activity phosphoadenosine phosphosulfate reductase is increased or generated in a non-human organism, like a microorganism or a plant cell, plant or part thereof, especially with mitochondrial localization, whereby the respective line discloses in column 7 citrulline. For example, an increase of the citrulline of at least 1 percent, particularly in a range of 32 to 95-percent is conferred as compared to a corresponding non-transformed wild type non-human organism.

Accordingly, in one embodiment, an increase of asparagine in a non-human organism, as compared to a corresponding non-transformed wild type non-human organism, is conferred in the process of the invention, if the activity of a polypeptide showing the activity of a riboflavin biosynthesis protein, or if the activity of the polypeptide Slr1882, preferably represented by SEQ ID NO. 59042, or a homolog or fragment thereof, or if the activity of a polypeptide encoded by a nucleic acid molecule comprising the nucleic acid SEQ ID NO. 59041, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 59041 or polypeptide SEQ ID NO. 59042, respectively, or a homolog or a fragment thereof, is increased or generated, or if the activity riboflavin biosynthesis protein is increased or generated in a non-human organism, like a microorganism or a plant cell, plant or part thereof, especially with non-targeted localization, whereby the respective line discloses in column 7 asparagine. For example, an increase of the asparagine of at least 1 percent, particularly in a range of 65 to 112-percent is conferred as compared to a corresponding non-transformed wild type non-human organism.

Accordingly, in one embodiment, an increase of homoserine in a non-human organism, as compared to a corresponding non-transformed wild type non-human organism, is conferred in the process of the invention, if the activity of a polypeptide showing the activity of a threonine dehydratase, or if the activity of the polypeptide Slr2072, preferably represented by SEQ ID NO. 12342, or a homolog or fragment thereof, or if the activity of a polypeptide encoded by a nucleic acid molecule comprising the nucleic acid SEQ ID NO. 12341, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 12341 or polypeptide SEQ ID NO. 12342, respectively, or a homolog or a fragment thereof, is increased or generated, or if the activity threonine dehydratase is increased or generated in a non-human organism, like a microorganism or a plant cell, plant or part thereof, especially with plastidic localization, whereby the respective line discloses in column 7 homoserine. For example, an increase of the homoserine of at least 1 percent, particularly in a range of 40 to 86-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a threonine dehydratase, or if the activity of the polypeptide Slr2072, preferably represented by SEQ ID NO. 12342, or a homolog or fragment thereof, or if the activity of a polypeptide encoded by a nucleic acid molecule comprising the nucleic acid SEQ ID NO. 12341, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 12341 or polypeptide SEQ ID NO. 12342, respectively, or a homolog or a fragment thereof, is increased or generated, or if the activity threonine dehydratase is increased or generated in a non-human organism, like a microorganism or a plant cell, plant or part thereof, especially with mitochondrial localization, whereby the respective line discloses in column 7 tyrosine. For example, an increase of the tyrosine of at least 1 percent, particularly in a range of 30 to 65-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a short-chain alcohol dehydrogenase family, or if the activity of the polypeptide Slr2124, preferably represented by SEQ ID NO. 59371, or a homolog or fragment thereof, or if the activity of a polypeptide encoded by a nucleic acid molecule comprising the nucleic acid SEQ ID NO. 59370, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 59370 or polypeptide SEQ ID NO. 59371, respectively, or a homolog or a fragment thereof, is increased or generated, or if the activity short-chain alcohol dehydrogenase family is increased or generated in a non-human organism, like a microorganism or a plant cell, plant or part thereof, especially with plastidic localization, whereby the respective line discloses in column 7 alanine. For example, an increase of the alanine of at least 1 percent, particularly in a range of 19 to 44-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a short-chain alcohol dehydrogenase family, or if the activity of the polypeptide Slr2124, preferably represented by SEQ ID NO. 59371, or a homolog or fragment thereof, or if the activity of a polypeptide encoded by a nucleic acid molecule comprising the nucleic acid SEQ ID NO. 59370, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 59370 or polypeptide SEQ ID NO. 59371, respectively, or a homolog or a fragment thereof, is increased or generated, or if the activity short-chain alcohol dehydrogenase family is increased or generated in a non-human organism, like a microorganism or a plant cell, plant or part thereof, especially with plastidic localization, whereby the respective line discloses in column 7 phenylalanine. For example, an increase of the phenylalanine of at least 1 percent, particularly in a range of 36 to 69-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a short-chain alcohol dehydrogenase family, or if the activity of the polypeptide Slr2124, preferably represented by SEQ ID NO. 59371, or a homolog or fragment thereof, or if the activity of a polypeptide encoded by a nucleic acid molecule comprising the nucleic acid SEQ ID NO. 59370, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 59370 or polypeptide SEQ ID NO. 59371, respectively, or a homolog or a fragment thereof, is increased or generated, or if the activity short-chain alcohol dehydrogenase family is increased or generated in a non-human organism, like a microorganism or a plant cell, plant or part thereof, especially with plastidic localization, whereby the respective line discloses in column 7 tyrosine. For example, an increase of the tyrosine of at least 1 percent, particularly in a range of 36 to 87-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a Sec-independent protein translocase subunit, or if the activity of the polypeptide TTC0019, preferably represented by SEQ ID NO. 12699, or a homolog or fragment thereof, or if the activity of a polypeptide encoded by a nucleic acid molecule comprising the nucleic acid SEQ ID NO. 12698, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 12698 or polypeptide SEQ ID NO. 12699, respectively, or a homolog or a fragment thereof, is increased or generated, or if the activity Sec-independent protein translocase subunit is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 31 to 624-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a Sec-independent protein translocase subunit, or if the activity of the polypeptide TTC0019, preferably represented by SEQ ID NO. 12699, or a homolog or fragment thereof, or if the activity of a polypeptide encoded by a nucleic acid molecule comprising the nucleic acid SEQ ID NO. 12698, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 12698 or polypeptide SEQ ID NO. 12699, respectively, or a homolog or a fragment thereof, is increased or generated, or if the activity Sec-independent protein translocase subunit is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 157 to 378-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a Sec-independent protein translocase subunit, or if the activity of the polypeptide TTC0019, preferably represented by SEQ ID NO. 12699, or a homolog or fragment thereof, or if the activity of a polypeptide encoded by a nucleic acid molecule comprising the nucleic acid SEQ ID NO. 12698, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 12698 or polypeptide SEQ ID NO. 12699, respectively, or a homolog or a fragment thereof, is increased or generated, or if the activity Sec-independent protein translocase subunit is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 36 to 277-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a transcriptional regulator, or if the activity of the polypeptide TTC0239, preferably represented by SEQ ID NO. 88527, or a homolog or fragment thereof, or if the activity of a polypeptide encoded by a nucleic acid molecule comprising the nucleic acid SEQ ID NO. 88526, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 88526 or polypeptide SEQ ID NO. 88527, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 35 to 136-percent is conferred as compared to a corresponding non-transformed wild type non-human organism.

Accordingly, in one embodiment, an increase of asparagine in a non-human organism, as compared to a corresponding non-transformed wild type non-human organism, is conferred in the process of the invention, if the activity of a polypeptide showing the activity of a amino acid ABC transporter permease protein, or if the activity of the polypeptide TTC0337, preferably represented by SEQ ID NO. 61071, or a homolog or fragment thereof, or if the activity of a polypeptide encoded by a nucleic acid molecule comprising the nucleic acid SEQ ID NO. 61070, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 61070 or polypeptide SEQ ID NO. 61071, respectively, or a homolog or a fragment thereof, is increased or generated, or if the activity amino acid ABC transporter permease protein is increased or generated in a non-human organism, like a microorganism or a plant cell, plant or part thereof, especially with non-targeted localization, whereby the respective line discloses in column 7 asparagine. For example, an increase of the asparagine of at least 1 percent, particularly in a range of 44 to 216-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a TTC0768-protein, or if the activity of the polypeptide TTC0768, preferably represented by SEQ ID NO. 61533, or a homolog or fragment thereof, or if the activity of a polypeptide encoded by a nucleic acid molecule comprising the nucleic acid SEQ ID NO. 61532, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 61532 or polypeptide SEQ ID NO. 61533, respectively, or a homolog or a fragment thereof, is increased or generated, or if the activity TTC0768-protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 51 to 97-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a TTC0881-protein, or if the activity of the polypeptide TTC0881, preferably represented by SEQ ID NO. 88876, or a homolog or fragment thereof, or if the activity of a polypeptide encoded by a nucleic acid molecule comprising the nucleic acid SEQ ID NO. 88875, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 88875 or polypeptide SEQ ID NO. 88876, respectively, or a homolog or a fragment thereof, is increased or generated, or if the activity TTC0881-protein is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 25 to 40-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a TTC0881-protein, or if the activity of the polypeptide TTC0881, preferably represented by SEQ ID NO. 88876, or a homolog or fragment thereof, or if the activity of a polypeptide encoded by a nucleic acid molecule comprising the nucleic acid SEQ ID NO. 88875, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 88875 or polypeptide SEQ ID NO. 88876, respectively, or a homolog or a fragment thereof, is increased or generated, or if the activity TTC0881-protein is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 27 to 84-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a metal-dependent hydrolase, or if the activity of the polypeptide TTC0917, preferably represented by SEQ ID NO. 61554, or a homolog or fragment thereof, or if the activity of a polypeptide encoded by a nucleic acid molecule comprising the nucleic acid SEQ ID NO. 61553, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 61553 or polypeptide SEQ ID NO. 61554, respectively, or a homolog or a fragment thereof, is increased or generated, or if the activity metal-dependent hydrolase is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 20 to 37-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a metal-dependent hydrolase, or if the activity of the polypeptide TTC0917, preferably represented by SEQ ID NO. 61554, or a homolog or fragment thereof, or if the activity of a polypeptide encoded by a nucleic acid molecule comprising the nucleic acid SEQ ID NO. 61553, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 61553 or polypeptide SEQ ID NO. 61554, respectively, or a homolog or a fragment thereof, is increased or generated, or if the activity metal-dependent hydrolase is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 23 to 35-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a multiple antibiotic resistance protein, or if the activity of the polypeptide TTC1193, preferably represented by SEQ ID NO. 61724, or a homolog or fragment thereof, or if the activity of a polypeptide encoded by a nucleic acid molecule comprising the nucleic acid SEQ ID NO. 61723, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 61723 or polypeptide SEQ ID NO. 61724, respectively, or a homolog or a fragment thereof, is increased or generated, or if the activity multiple antibiotic resistance protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 33 to 133-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a multiple antibiotic resistance protein, or if the activity of the polypeptide TTC1193, preferably represented by SEQ ID NO. 61724, or a homolog or fragment thereof, or if the activity of a polypeptide encoded by a nucleic acid molecule comprising the nucleic acid SEQ ID NO. 61723, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 61723 or polypeptide SEQ ID NO. 61724, respectively, or a homolog or a fragment thereof, is increased or generated, or if the activity multiple antibiotic resistance protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 53 to 103-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a TTC1386-protein, or if the activity of the polypeptide TTC1386, preferably represented by SEQ ID NO. 62080, or a homolog or fragment thereof, or if the activity of a polypeptide encoded by a nucleic acid molecule comprising the nucleic acid SEQ ID NO. 62079, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 62079 or polypeptide SEQ ID NO. 62080, respectively, or a homolog or a fragment thereof, is increased or generated, or if the activity TTC1386-protein is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 54 to 91-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a TTC1386-protein, or if the activity of the polypeptide TTC1386, preferably represented by SEQ ID NO. 62080, or a homolog or fragment thereof, or if the activity of a polypeptide encoded by a nucleic acid molecule comprising the nucleic acid SEQ ID NO. 62079, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 62079 or polypeptide SEQ ID NO. 62080, respectively, or a homolog or a fragment thereof, is increased or generated, or if the activity TTC1386-protein is increased or generated in a non-human organism, like a microorganism or a plant cell, plant or part thereof, especially with non-targeted localization, whereby the respective line discloses in column 7 glycine. For example, an increase of the glycine of at least 1 percent, particularly in a range of 230 to 978-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a TTC1386-protein, or if the activity of the polypeptide TTC1386, preferably represented by SEQ ID NO. 62080, or a homolog or fragment thereof, or if the activity of a polypeptide encoded by a nucleic acid molecule comprising the nucleic acid SEQ ID NO. 62079, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 62079 or polypeptide SEQ ID NO. 62080, respectively, or a homolog or a fragment thereof, is increased or generated, or if the activity TTC1386-protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 45 to 528-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a TTC1386-protein, or if the activity of the polypeptide TTC1386, preferably represented by SEQ ID NO. 62080, or a homolog or fragment thereof, or if the activity of a polypeptide encoded by a nucleic acid molecule comprising the nucleic acid SEQ ID NO. 62079, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 62079 or polypeptide SEQ ID NO. 62080, respectively, or a homolog or a fragment thereof, is increased or generated, or if the activity TTC1386-protein is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 23 to 117-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a TTC1386-protein, or if the activity of the polypeptide TTC1386, preferably represented by SEQ ID NO. 62080, or a homolog or fragment thereof, or if the activity of a polypeptide encoded by a nucleic acid molecule comprising the nucleic acid SEQ ID NO. 62079, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 62079 or polypeptide SEQ ID NO. 62080, respectively, or a homolog or a fragment thereof, is increased or generated, or if the activity TTC1386-protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 39 to 829-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a homocitrate synthase, or if the activity of the polypeptide TTC1550, preferably represented by SEQ ID NO. 12975, or a homolog or fragment thereof, or if the activity of a polypeptide encoded by a nucleic acid molecule comprising the nucleic acid SEQ ID NO. 12974, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 12974 or polypeptide SEQ ID NO. 12975, respectively, or a homolog or a fragment thereof, is increased or generated, or if the activity homocitrate synthase is increased or generated in a non-human organism, like a microorganism or a plant cell, plant or part thereof, especially with non-targeted localization, whereby the respective line discloses in column 7 alanine. For example, an increase of the alanine of at least 1 percent, particularly in a range of 45 to 86-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a homocitrate synthase, or if the activity of the polypeptide TTC1550, preferably represented by SEQ ID NO. 12975, or a homolog or fragment thereof, or if the activity of a polypeptide encoded by a nucleic acid molecule comprising the nucleic acid SEQ ID NO. 12974, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 12974 or polypeptide SEQ ID NO. 12975, respectively, or a homolog or a fragment thereof, is increased or generated, or if the activity homocitrate synthase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 54 to 266-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a homocitrate synthase, or if the activity of the polypeptide TTC1550, preferably represented by SEQ ID NO. 12975, or a homolog or fragment thereof, or if the activity of a polypeptide encoded by a nucleic acid molecule comprising the nucleic acid SEQ ID NO. 12974, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 12974 or polypeptide SEQ ID NO. 12975, respectively, or a homolog or a fragment thereof, is increased or generated, or if the activity homocitrate synthase is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 23 to 104-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a homocitrate synthase, or if the activity of the polypeptide TTC1550, preferably represented by SEQ ID NO. 12975, or a homolog or fragment thereof, or if the activity of a polypeptide encoded by a nucleic acid molecule comprising the nucleic acid SEQ ID NO. 12974, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 12974 or polypeptide SEQ ID NO. 12975, respectively, or a homolog or a fragment thereof, is increased or generated, or if the activity homocitrate synthase is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 35 to 213-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a homocitrate synthase, or if the activity of the polypeptide TTC1550, preferably represented by SEQ ID NO. 12975, or a homolog or fragment thereof, or if the activity of a polypeptide encoded by a nucleic acid molecule comprising the nucleic acid SEQ ID NO. 12974, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 12974 or polypeptide SEQ ID NO. 12975, respectively, or a homolog or a fragment thereof, is increased or generated, or if the activity homocitrate synthase is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 47 to 171-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a oxidoreductase subunit, or if the activity of the polypeptide TTC1918, preferably represented by SEQ ID NO. 62161, or a homolog or fragment thereof, or if the activity of a polypeptide encoded by a nucleic acid molecule comprising the nucleic acid SEQ ID NO. 62160, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 62160 or polypeptide SEQ ID NO. 62161, respectively, or a homolog or a fragment thereof, is increased or generated, or if the activity oxidoreductase subunit is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 50 to 141-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a oxidoreductase subunit, or if the activity of the polypeptide TTC1918, preferably represented by SEQ ID NO. 62161, or a homolog or fragment thereof, or if the activity of a polypeptide encoded by a nucleic acid molecule comprising the nucleic acid SEQ ID NO. 62160, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 62160 or polypeptide SEQ ID NO. 62161, respectively, or a homolog or a fragment thereof, is increased or generated, or if the activity oxidoreductase subunit is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 24 to 44-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a XM_{—}473199-protein, or if the activity of the polypeptide XM_{—}473199, preferably represented by SEQ ID NO. 62245, or a homolog or fragment thereof, or if the activity of a polypeptide encoded by a nucleic acid molecule comprising the nucleic acid SEQ ID NO. 62244, preferably the coding region thereof, or a homolog or fragment thereof, e.g. derived from Oryza sativa, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 62244 or polypeptide SEQ ID NO. 62245, respectively, or a homolog or a fragment thereof, is increased or generated, or if the activity XM_{—}473199-protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 53 to 91-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a XM_{—}473199-protein, or if the activity of the polypeptide XM_{—}473199, preferably represented by SEQ ID NO. 62245, or a homolog or fragment thereof, or if the activity of a polypeptide encoded by a nucleic acid molecule comprising the nucleic acid SEQ ID NO. 62244, preferably the coding region thereof, or a homolog or fragment thereof, e.g. derived from Oryza sativa, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 62244 or polypeptide SEQ ID NO. 62245, respectively, or a homolog or a fragment thereof, is increased or generated, or if the activity XM_{—}473199-protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 31 to 44-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a XM_{—}473199-protein, or if the activity of the polypeptide XM_{—}473199, preferably represented by SEQ ID NO. 62245, or a homolog or fragment thereof, or if the activity of a polypeptide encoded by a nucleic acid molecule comprising the nucleic acid SEQ ID NO. 62244, preferably the coding region thereof, or a homolog or fragment thereof, e.g. derived from Oryza sativa, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 62244 or polypeptide SEQ ID NO. 62245, respectively, or a homolog or a fragment thereof, is increased or generated, or if the activity XM_{—}473199-protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 35 to 47-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a XM_{—}473199-protein, or if the activity of the polypeptide XM_{—}473199, preferably represented by SEQ ID NO. 62245, or a homolog or fragment thereof, or if the activity of a polypeptide encoded by a nucleic acid molecule comprising the nucleic acid SEQ ID NO. 62244, preferably the coding region thereof, or a homolog or fragment thereof, e.g. derived from Oryza sativa, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 62244 or polypeptide SEQ ID NO. 62245, respectively, or a homolog or a fragment thereof, is increased or generated, or if the activity XM_{—}473199-protein is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 32 to 86-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a transcription factor, or if the activity of the polypeptide Ybl021 c, preferably represented by SEQ ID NO. 62525, or a homolog or fragment thereof, or if the activity of a polypeptide encoded by a nucleic acid molecule comprising the nucleic acid SEQ ID NO. 62524, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 62524 or polypeptide SEQ ID NO. 62525, respectively, or a homolog or a fragment thereof, is increased or generated, or if the activity transcription factor is increased or generated in a non-human organism, like a microorganism or a plant cell, plant or part thereof, especially with non-targeted localization, whereby the respective line discloses in column 7 glycine. For example, an increase of the glycine of at least 1 percent, particularly in a range of 42 to 325-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a transcription factor, or if the activity of the polypeptide Ybl021 c, preferably represented by SEQ ID NO. 62525, or a homolog or fragment thereof, or if the activity of a polypeptide encoded by a nucleic acid molecule comprising the nucleic acid SEQ ID NO. 62524, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 62524 or polypeptide SEQ ID NO. 62525, respectively, or a homolog or a fragment thereof, is increased or generated, or if the activity transcription factor is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 31 to 40-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a cell division control protein, or if the activity of the polypeptide Ybr160w, preferably represented by SEQ ID NO. 62718, or a homolog or fragment thereof, or if the activity of a polypeptide encoded by a nucleic acid molecule comprising the nucleic acid SEQ ID NO. 62717, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 62717 or polypeptide SEQ ID NO. 62718, respectively, or a homolog or a fragment thereof, is increased or generated, or if the activity cell division control protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 53 to 86-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a cell division control protein, or if the activity of the polypeptide Ybr160w, preferably represented by SEQ ID NO. 62718, or a homolog or fragment thereof, or if the activity of a polypeptide encoded by a nucleic acid molecule comprising the nucleic acid SEQ ID NO. 62717, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 62717 or polypeptide SEQ ID NO. 62718, respectively, or a homolog or a fragment thereof, is increased or generated, or if the activity cell division control protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 64 to 100-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a 3-deoxy-7-phosphoheptulonate synthase, or if the activity of the polypeptide Ybr249c, preferably represented by SEQ ID NO. 88880, or a homolog or fragment thereof, or if the activity of a polypeptide encoded by a nucleic acid molecule comprising the nucleic acid SEQ ID NO. 88879, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 88879 or polypeptide SEQ ID NO. 88880, respectively, or a homolog or a fragment thereof, is increased or generated, or if the activity 3-deoxy-7-phosphoheptulonate synthase is increased or generated in a non-human organism, like a microorganism or a plant cell, plant or part thereof, especially with plastidic localization, whereby the respective line discloses in column 7 phenylalanine. For example, an increase of the phenylalanine of at least 1 percent, particularly in a range of 210 to 5005-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a 3-deoxy-7-phosphoheptulonate synthase, or if the activity of the polypeptide Ybr249c, preferably represented by SEQ ID NO. 88880, or a homolog or fragment thereof, or if the activity of a polypeptide encoded by a nucleic acid molecule comprising the nucleic acid SEQ ID NO. 88879, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 88879 or polypeptide SEQ ID NO. 88880, respectively, or a homolog or a fragment thereof, is increased or generated, or if the activity 3-deoxy-7-phosphoheptulonate synthase is increased or generated in a non-human organism, like a microorganism or a plant cell, plant or part thereof, especially with plastidic localization, whereby the respective line discloses in column 7 tyrosine. For example, an increase of the tyrosine of at least 1 percent, particularly in a range of 159 to 2254-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a phosphate permease, or if the activity of the polypeptide Ybr296c, preferably represented by SEQ ID NO. 89318, or a homolog or fragment thereof, or if the activity of a polypeptide encoded by a nucleic acid molecule comprising the nucleic acid SEQ ID NO. 89317, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 89317 or polypeptide SEQ ID NO. 89318, respectively, or a homolog or a fragment thereof, is increased or generated, or if the activity phosphate permease is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 33 to 51-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a serine/threonine dehydratase, or if the activity of the polypeptide Ycl064c, preferably represented by SEQ ID NO. 89396, or a homolog or fragment thereof, or if the activity of a polypeptide encoded by a nucleic acid molecule comprising the nucleic acid SEQ ID NO. 89395, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 89395 or polypeptide SEQ ID NO. 89396, respectively, or a homolog or a fragment thereof, is increased or generated, or if the activity serine/threonine dehydratase is increased or generated in a non-human organism, like a microorganism or a plant cell, plant or part thereof, especially with plastidic localization, whereby the respective line discloses in column 7 alanine. For example, an increase of the alanine of at least 1 percent, particularly in a range of 23 to 51-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a serine/threonine dehydratase, or if the activity of the polypeptide Yc1064c, preferably represented by SEQ ID NO. 89396, or a homolog or fragment thereof, or if the activity of a polypeptide encoded by a nucleic acid molecule comprising the nucleic acid SEQ ID NO. 89395, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 89395 or polypeptide SEQ ID NO. 89396, respectively, or a homolog or a fragment thereof, is increased or generated, or if the activity serine/threonine dehydratase is increased or generated in a non-human organism, like a microorganism or a plant cell, plant or part thereof, especially with plastidic localization, whereby the respective line discloses in column 7 glycine. For example, an increase of the glycine of at least 1 percent, particularly in a range of 36 to 106-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a protein kinase, or if the activity of the polypeptide Ydl159w, preferably represented by SEQ ID NO. 14276, or a homolog or fragment thereof, or if the activity of a polypeptide encoded by a nucleic acid molecule comprising the nucleic acid SEQ ID NO. 14275, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 14275 or polypeptide SEQ ID NO. 14276, respectively, or a homolog or a fragment thereof, is increased or generated, or if the activity protein kinase is increased or generated in a non-human organism, like a microorganism or a plant cell, plant or part thereof, especially with plastidic localization, whereby the respective line discloses in column 7 aspartate. For example, an increase of the aspartate of at least 1 percent, particularly in a range of 70 to 87-percent is conferred as compared to a corresponding non-transformed wild type non-human organism.

Accordingly, in one embodiment, an increase of homoserine in a non-human organism, as compared to a corresponding non-transformed wild type non-human organism, is conferred in the process of the invention, if the activity of a polypeptide showing the activity of a Serine/threonine-protein phosphatase, or if the activity of the polypeptide Ydl188c, preferably represented by SEQ ID NO. 89484, or a homolog or fragment thereof, or if the activity of a polypeptide encoded by a nucleic acid molecule comprising the nucleic acid SEQ ID NO. 89483, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 89483 or polypeptide SEQ ID NO. 89484, respectively, or a homolog or a fragment thereof, is increased or generated, or if the activity Serine/threonine-protein phosphatase is increased or generated in a non-human organism, like a microorganism or a plant cell, plant or part thereof, especially with plastidic localization, whereby the respective line discloses in column 7 homoserine. For example, an increase of the homoserine of at least 1 percent, particularly in a range of 36 to 75-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a Serine/threonine-protein phosphatase, or if the activity of the polypeptide Ydl188c, preferably represented by SEQ ID NO. 89484, or a homolog or fragment thereof, or if the activity of a polypeptide encoded by a nucleic acid molecule comprising the nucleic acid SEQ ID NO. 89483, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 89483 or polypeptide SEQ ID NO. 89484, respectively, or a homolog or a fragment thereof, is increased or generated, or if the activity Serine/threonine-protein phosphatase is increased or generated in a non-human organism, like a microorganism or a plant cell, plant or part thereof, especially with plastidic localization, whereby the respective line discloses in column 7 phenylalanine. For example, an increase of the phenylalanine of at least 1 percent, particularly in a range of 28 to 41-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a Pre-mRNA-splicing factor, or if the activity of the polypeptide Ydl209c, preferably represented by SEQ ID NO. 89937, or a homolog or fragment thereof, or if the activity of a polypeptide encoded by a nucleic acid molecule comprising the nucleic acid SEQ ID NO. 89936, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 89936 or polypeptide SEQ ID NO. 89937, respectively, or a homolog or a fragment thereof, is increased or generated, or if the activity Pre-mRNA-splicing factor is increased or generated in a non-human organism, like a microorganism or a plant cell, plant or part thereof, especially with plastidic localization, whereby the respective line discloses in column 7 5-oxoproline. For example, an increase of the 5-oxoproline of at least 1 percent, particularly in a range of 46 to 97-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a N-(5′-phosphoribosyl)anthranilate isomerase, or if the activity of the polypeptide Ydr007w, preferably represented by SEQ ID NO. 89974, or a homolog or fragment thereof, or if the activity of a polypeptide encoded by a nucleic acid molecule comprising the nucleic acid SEQ ID NO. 89973, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 89973 or polypeptide SEQ ID NO. 89974, respectively, or a homolog or a fragment thereof, is increased or generated, or if the activity N-(5′-phosphoribosyl)anthranilate isomerase is increased or generated in a non-human organism, like a microorganism or a plant cell, plant or part thereof, especially with plastidic localization, whereby the respective line discloses in column 7 phenylalanine. For example, an increase of the phenylalanine of at least 1 percent, particularly in a range of 31 to 50-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a branched-chain amino acid permease, or if the activity of the polypeptide Ydr046c, preferably represented by SEQ ID NO. 63545, or a homolog or fragment thereof, or if the activity of a polypeptide encoded by a nucleic acid molecule comprising the nucleic acid SEQ ID NO. 63544, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 63544 or polypeptide SEQ ID NO. 63545, respectively, or a homolog or a fragment thereof, is increased or generated, or if the activity branched-chain amino acid permease is increased or generated in a non-human organism, like a microorganism or a plant cell, plant or part thereof, especially with plastidic localization, whereby the respective line discloses in column 7 tyrosine. For example, an increase of the tyrosine of at least 1 percent, particularly in a range of 39 to 95-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a ydr183w-protein, or if the activity of the polypeptide Ydr183w, preferably represented by SEQ ID NO. 90104, or a homolog or fragment thereof, or if the activity of a polypeptide encoded by a nucleic acid molecule comprising the nucleic acid SEQ ID NO. 90103, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 90103 or polypeptide SEQ ID NO. 90104, respectively, or a homolog or a fragment thereof, is increased or generated, or if the activity ydr183w-protein is increased or generated in a non-human organism, like a microorganism or a plant cell, plant or part thereof, especially with plastidic localization, whereby the respective line discloses in column 7 serine. For example, an increase of the serine of at least 1 percent, particularly in a range of 25 to 80-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a ydr273w-protein, or if the activity of the polypeptide Ydr273w, preferably represented by SEQ ID NO. 90160, or a homolog or fragment thereof, or if the activity of a polypeptide encoded by a nucleic acid molecule comprising the nucleic acid SEQ ID NO. 90159, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 90159 or polypeptide SEQ ID NO. 90160, respectively, or a homolog or a fragment thereof, is increased or generated, or if the activity ydr273w-protein is increased or generated in a non-human organism, like a microorganism or a plant cell, plant or part thereof, especially with plastidic localization, whereby the respective line discloses in column 7 aspartate. For example, an increase of the aspartate of at least 1 percent, particularly in a range of 23 to 52-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a ydr273w-protein, or if the activity of the polypeptide Ydr273w, preferably represented by SEQ ID NO. 90160, or a homolog or fragment thereof, or if the activity of a polypeptide encoded by a nucleic acid molecule comprising the nucleic acid SEQ ID NO. 90159, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 90159 or polypeptide SEQ ID NO. 90160, respectively, or a homolog or a fragment thereof, is increased or generated, or if the activity ydr273w-protein is increased or generated in a non-human organism, like a microorganism or a plant cell, plant or part thereof, especially with plastidic localization, whereby the respective line discloses in column 7 citrulline. For example, an increase of the citrulline of at least 1 percent, particularly in a range of 34 to 36-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a ydr507c-protein, or if the activity of the polypeptide Ydr507c, preferably represented by SEQ ID NO. 90166, or a homolog or fragment thereof, or if the activity of a polypeptide encoded by a nucleic acid molecule comprising the nucleic acid SEQ ID NO. 90165, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 90165 or polypeptide SEQ ID NO. 90166, respectively, or a homolog or a fragment thereof, is increased or generated, or if the activity ydr507c-protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 64 to 78-percent is conferred as compared to a corresponding non-transformed wild type non-human organism.

Accordingly, in one embodiment, an increase of asparagine in a non-human organism, as compared to a corresponding non-transformed wild type non-human organism, is conferred in the process of the invention, if the activity of a polypeptide showing the activity of a yer014w-protein, or if the activity of the polypeptide Yer014w, preferably represented by SEQ ID NO. 63746, or a homolog or fragment thereof, or if the activity of a polypeptide encoded by a nucleic acid molecule comprising the nucleic acid SEQ ID NO. 63745, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 63745 or polypeptide SEQ ID NO. 63746, respectively, or a homolog or a fragment thereof, is increased or generated, or if the activity yer014w-protein is increased or generated in a non-human organism, like a microorganism or a plant cell, plant or part thereof, especially with plastidic localization, whereby the respective line discloses in column 7 asparagine. For example, an increase of the asparagine of at least 1 percent, particularly in a range of 46 to 159-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a yer014w-protein, or if the activity of the polypeptide Yer014w, preferably represented by SEQ ID NO. 63746, or a homolog or fragment thereof, or if the activity of a polypeptide encoded by a nucleic acid molecule comprising the nucleic acid SEQ ID NO. 63745, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 63745 or polypeptide SEQ ID NO. 63746, respectively, or a homolog or a fragment thereof, is increased or generated, or if the activity yer014w-protein is increased or generated in a non-human organism, like a microorganism or a plant cell, plant or part thereof, especially with plastidic localization, whereby the respective line discloses in column 7 aspartate. For example, an increase of the aspartate of at least 1 percent, particularly in a range of 48 to 84-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a yer014w-protein, or if the activity of the polypeptide Yer014w, preferably represented by SEQ ID NO. 63746, or a homolog or fragment thereof, or if the activity of a polypeptide encoded by a nucleic acid molecule comprising the nucleic acid SEQ ID NO. 63745, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 63745 or polypeptide SEQ ID NO. 63746, respectively, or a homolog or a fragment thereof, is increased or generated, or if the activity yer014w-protein is increased or generated in a non-human organism, like a microorganism or a plant cell, plant or part thereof, especially with plastidic localization, whereby the respective line discloses in column 7 serine. For example, an increase of the serine of at least 1 percent, particularly in a range of 25 to 82-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a yer106w-protein, or if the activity of the polypeptide Yer106w, preferably represented by SEQ ID NO. 63804, or a homolog or fragment thereof, or if the activity of a polypeptide encoded by a nucleic acid molecule comprising the nucleic acid SEQ ID NO. 63803, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 63803 or polypeptide SEQ ID NO. 63804, respectively, or a homolog or a fragment thereof, is increased or generated, or if the activity yer106w-protein is increased or generated in a non-human organism, like a microorganism or a plant cell, plant or part thereof, especially with plastidic localization, whereby the respective line discloses in column 7 citrulline. For example, an increase of the citrulline of at least 1 percent, particularly in a range of 34 to 85-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a molecular chaperone portein, or if the activity of the polypeptide Yfl016c, preferably represented by SEQ ID NO. 63808, or a homolog or fragment thereof, or if the activity of a polypeptide encoded by a nucleic acid molecule comprising the nucleic acid SEQ ID NO. 63807, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 63807 or polypeptide SEQ ID NO. 63808, respectively, or a homolog or a fragment thereof, is increased or generated, or if the activity molecular chaperone portein is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 23 to 57-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a molecular chaperone portein, or if the activity of the polypeptide Yfl016c, preferably represented by SEQ ID NO. 63808, or a homolog or fragment thereof, or if the activity of a polypeptide encoded by a nucleic acid molecule comprising the nucleic acid SEQ ID NO. 63807, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 63807 or polypeptide SEQ ID NO. 63808, respectively, or a homolog or a fragment thereof, is increased or generated, or if the activity molecular chaperone portein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 44 to 182-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a YFL019C-protein, or if the activity of the polypeptide Yfl019c, preferably represented by SEQ ID NO. 64145, or a homolog or fragment thereof, or if the activity of a polypeptide encoded by a nucleic acid molecule comprising the nucleic acid SEQ ID NO. 64144, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 64144 or polypeptide SEQ ID NO. 64145, respectively, or a homolog or a fragment thereof, is increased or generated, or if the activity YFL019C-protein is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 103 to 305-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a YFL019C-protein, or if the activity of the polypeptide Yfl019c, preferably represented by SEQ ID NO. 64145, or a homolog or fragment thereof, or if the activity of a polypeptide encoded by a nucleic acid molecule comprising the nucleic acid SEQ ID NO. 64144, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 64144 or polypeptide SEQ ID NO. 64145, respectively, or a homolog or a fragment thereof, is increased or generated, or if the activity YFL019C-protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 34 to 185-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a aminotransferase, or if the activity of the polypeptide Yfl030w, preferably represented by SEQ ID NO. 90190, or a homolog or fragment thereof, or if the activity of a polypeptide encoded by a nucleic acid molecule comprising the nucleic acid SEQ ID NO. 90189, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 90189 or polypeptide SEQ ID NO. 90190, respectively, or a homolog or a fragment thereof, is increased or generated, or if the activity aminotransferase is increased or generated in a non-human organism, like a microorganism or a plant cell, plant or part thereof, especially with plastidic localization, whereby the respective line discloses in column 7 alanine. For example, an increase of the alanine of at least 1 percent, particularly in a range of 29 to 33-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a aminotransferase, or if the activity of the polypeptide Yfl030w, preferably represented by SEQ ID NO. 90190, or a homolog or fragment thereof, or if the activity of a polypeptide encoded by a nucleic acid molecule comprising the nucleic acid SEQ ID NO. 90189, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 90189 or polypeptide SEQ ID NO. 90190, respectively, or a homolog or a fragment thereof, is increased or generated, or if the activity aminotransferase is increased or generated in a non-human organism, like a microorganism or a plant cell, plant or part thereof, especially with plastidic localization, whereby the respective line discloses in column 7 glycine. For example, an increase of the glycine of at least 1 percent, particularly in a range of 34 to 99-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a yfl054c-protein, or if the activity of the polypeptide Yfl054c, preferably represented by SEQ ID NO. 64149, or a homolog or fragment thereof, or if the activity of a polypeptide encoded by a nucleic acid molecule comprising the nucleic acid SEQ ID NO. 64148, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 64148 or polypeptide SEQ ID NO. 64149, respectively, or a homolog or a fragment thereof, is increased or generated, or if the activity yfl054c-protein is increased or generated in a non-human organism, like a microorganism or a plant cell, plant or part thereof, especially with plastidic localization, whereby the respective line discloses in column 7 phenylalanine. For example, an increase of the phenylalanine of at least 1 percent, particularly in a range of 36 to 39-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a yfl054c-protein, or if the activity of the polypeptide Yfl054c, preferably represented by SEQ ID NO. 64149, or a homolog or fragment thereof, or if the activity of a polypeptide encoded by a nucleic acid molecule comprising the nucleic acid SEQ ID NO. 64148, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 64148 or polypeptide SEQ ID NO. 64149, respectively, or a homolog or a fragment thereof, is increased or generated, or if the activity yfl054c-protein is increased or generated in a non-human organism, like a microorganism or a plant cell, plant or part thereof, especially with plastidic localization, whereby the respective line discloses in column 7 serine. For example, an increase of the serine of at least 1 percent, particularly in a range of 23 to 51-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a ygl096w-protein, or if the activity of the polypeptide Ygl096w, preferably represented by SEQ ID NO. 90279, or a homolog or fragment thereof, or if the activity of a polypeptide encoded by a nucleic acid molecule comprising the nucleic acid SEQ ID NO. 90278, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 90278 or polypeptide SEQ ID NO. 90279, respectively, or a homolog or a fragment thereof, is increased or generated, or if the activity ygl096w-protein is increased or generated in a non-human organism, like a microorganism or a plant cell, plant or part thereof, especially with plastidic localization, whereby the respective line discloses in column 7 5-oxoproline. For example, an increase of the 5-oxoproline of at least 1 percent, particularly in a range of 34 to 133-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a ygl237c-protein, or if the activity of the polypeptide Ygl237c, preferably represented by SEQ ID NO. 64178, or a homolog or fragment thereof, or if the activity of a polypeptide encoded by a nucleic acid molecule comprising the nucleic acid SEQ ID NO. 64177, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 64177 or polypeptide SEQ ID NO. 64178, respectively, or a homolog or a fragment thereof, is increased or generated, or if the activity ygl237c-protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 45 to 166-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a ygr221c-protein, or if the activity of the polypeptide Ygr221c, preferably represented by SEQ ID NO. 64316, or a homolog or fragment thereof, or if the activity of a polypeptide encoded by a nucleic acid molecule comprising the nucleic acid SEQ ID NO. 64315, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 64315 or polypeptide SEQ ID NO. 64316, respectively, or a homolog or a fragment thereof, is increased or generated, or if the activity ygr221c-protein is increased or generated in a non-human organism, like a microorganism or a plant cell, plant or part thereof, especially with plastidic localization, whereby the respective line discloses in column 7 aspartate. For example, an increase of the aspartate of at least 1 percent, particularly in a range of 50 to 98-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a ygr221c-protein, or if the activity of the polypeptide Ygr221c, preferably represented by SEQ ID NO. 64316, or a homolog or fragment thereof, or if the activity of a polypeptide encoded by a nucleic acid molecule comprising the nucleic acid SEQ ID NO. 64315, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 64315 or polypeptide SEQ ID NO. 64316, respectively, or a homolog or a fragment thereof, is increased or generated, or if the activity ygr221c-protein is increased or generated in a non-human organism, like a microorganism or a plant cell, plant or part thereof, especially with plastidic localization, whereby the respective line discloses in column 7 5-oxoproline. For example, an increase of the 5-oxoproline of at least 1 percent, particularly in a range of 40 to 44-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a yhl013c-protein, or if the activity of the polypeptide YhI013c, preferably represented by SEQ ID NO. 14716, or a homolog or fragment thereof, or if the activity of a polypeptide encoded by a nucleic acid molecule comprising the nucleic acid SEQ ID NO. 14715, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 14715 or polypeptide SEQ ID NO. 14716, respectively, or a homolog or a fragment thereof, is increased or generated, or if the activity yhl013c-protein is increased or generated in a non-human organism, like a microorganism or a plant cell, plant or part thereof, especially with plastidic localization, whereby the respective line discloses in column 7 phenylalanine. For example, an increase of the phenylalanine of at least 1 percent, particularly in a range of 35 to 410-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a yhr006w-protein, or if the activity of the polypeptide Yhr006w, preferably represented by SEQ ID NO. 90285, or a homolog or fragment thereof, or if the activity of a polypeptide encoded by a nucleic acid molecule comprising the nucleic acid SEQ ID NO. 90284, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 90284 or polypeptide SEQ ID NO. 90285, respectively, or a homolog or a fragment thereof, is increased or generated, or if the activity yhr006w-protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 32 to 67-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a yhr006w-protein, or if the activity of the polypeptide Yhr006w, preferably represented by SEQ ID NO. 90285, or a homolog or fragment thereof, or if the activity of a polypeptide encoded by a nucleic acid molecule comprising the nucleic acid SEQ ID NO. 90284, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 90284 or polypeptide SEQ ID NO. 90285, respectively, or a homolog or a fragment thereof, is increased or generated, or if the activity yhr006w-protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 39 to 129-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a yhr207c-protein, or if the activity of the polypeptide Yhr207c, preferably represented by SEQ ID NO. 64547, or a homolog or fragment thereof, or if the activity of a polypeptide encoded by a nucleic acid molecule comprising the nucleic acid SEQ ID NO. 64546, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 64546 or polypeptide SEQ ID NO. 64547, respectively, or a homolog or a fragment thereof, is increased or generated, or if the activity yhr207c-protein is increased or generated in a non-human organism, like a microorganism or a plant cell, plant or part thereof, especially with plastidic localization, whereby the respective line discloses in column 7 aspartate. For example, an increase of the aspartate of at least 1 percent, particularly in a range of 52 to 81-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a 3-phosphoglycerate dehydrogenase, or if the activity of the polypeptide Yil074c, preferably represented by SEQ ID NO. 64564, or a homolog or fragment thereof, or if the activity of a polypeptide encoded by a nucleic acid molecule comprising the nucleic acid SEQ ID NO. 64563, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 64563 or polypeptide SEQ ID NO. 64564, respectively, or a homolog or a fragment thereof, is increased or generated, or if the activity 3-phosphoglycerate dehydrogenase is increased or generated in a non-human organism, like a microorganism or a plant cell, plant or part thereof, especially with plastidic localization, whereby the respective line discloses in column 7 citrulline. For example, an increase of the citrulline of at least 1 percent, particularly in a range of 37 to 81-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a 3-phosphoglycerate dehydrogenase, or if the activity of the polypeptide Yil074c, preferably represented by SEQ ID NO. 64564, or a homolog or fragment thereof, or if the activity of a polypeptide encoded by a nucleic acid molecule comprising the nucleic acid SEQ ID NO. 64563, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 64563 or polypeptide SEQ ID NO. 64564, respectively, or a homolog or a fragment thereof, is increased or generated, or if the activity 3-phosphoglycerate dehydrogenase is increased or generated in a non-human organism, like a microorganism or a plant cell, plant or part thereof, especially with plastidic localization, whereby the respective line discloses in column 7 glycine. For example, an increase of the glycine of at least 1 percent, particularly in a range of 57 to 94-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a glutathione S-transferase, or if the activity of the polypeptide Yir038c, preferably represented by SEQ ID NO. 90307, or a homolog or fragment thereof, or if the activity of a polypeptide encoded by a nucleic acid molecule comprising the nucleic acid SEQ ID NO. 90306, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 90306 or polypeptide SEQ ID NO. 90307, respectively, or a homolog or a fragment thereof, is increased or generated, or if the activity glutathione S-transferase is increased or generated in a non-human organism, like a microorganism or a plant cell, plant or part thereof, especially with plastidic localization, whereby the respective line discloses in column 7 aspartate. For example, an increase of the aspartate of at least 1 percent, particularly in a range of 49 to 72-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a glutathione S-transferase, or if the activity of the polypeptide Yir038c, preferably represented by SEQ ID NO. 90307, or a homolog or fragment thereof, or if the activity of a polypeptide encoded by a nucleic acid molecule comprising the nucleic acid SEQ ID NO. 90306, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 90306 or polypeptide SEQ ID NO. 90307, respectively, or a homolog or a fragment thereof, is increased or generated, or if the activity glutathione S-transferase is increased or generated in a non-human organism, like a microorganism or a plant cell, plant or part thereof, especially with plastidic localization, whereby the respective line discloses in column 7 citrulline. For example, an increase of the citrulline of at least 1 percent, particularly in a range of 35 to 43-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a DnaJ-like chaperone, or if the activity of the polypeptide Yjl073w, preferably represented by SEQ ID NO. 64965, or a homolog or fragment thereof, or if the activity of a polypeptide encoded by a nucleic acid molecule comprising the nucleic acid SEQ ID NO. 64964, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 64964 or polypeptide SEQ ID NO. 64965, respectively, or a homolog or a fragment thereof, is increased or generated, or if the activity DnaJ-like chaperone is increased or generated in a non-human organism, like a microorganism or a plant cell, plant or part thereof, especially with plastidic localization, whereby the respective line discloses in column 7 aspartate. For example, an increase of the aspartate of at least 1 percent, particularly in a range of 52 to 62-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a cystathionine gamma-synthase, or if the activity of the polypeptide Yjr130c, preferably represented by SEQ ID NO. 14844, or a homolog or fragment thereof, or if the activity of a polypeptide encoded by a nucleic acid molecule comprising the nucleic acid SEQ ID NO. 14843, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 14843 or polypeptide SEQ ID NO. 14844, respectively, or a homolog or a fragment thereof, is increased or generated, or if the activity cystathionine gamma-synthase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 26 to 48-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a branched chain aminotransferase, or if the activity of the polypeptide Yjr148w, preferably represented by SEQ ID NO. 90410, or a homolog or fragment thereof, or if the activity of a polypeptide encoded by a nucleic acid molecule comprising the nucleic acid SEQ ID NO. 90409, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 90409 or polypeptide SEQ ID NO. 90410, respectively, or a homolog or a fragment thereof, is increased or generated, or if the activity branched chain aminotransferase is increased or generated in a non-human organism, like a microorganism or a plant cell, plant or part thereof, especially with plastidic localization, whereby the respective line discloses in column 7 citrulline. For example, an increase of the citrulline of at least 1 percent, particularly in a range of 31 to 67-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a polygalacturonase, or if the activity of the polypeptide Yjr153w, preferably represented by SEQ ID NO. 66275, or a homolog or fragment thereof, or if the activity of a polypeptide encoded by a nucleic acid molecule comprising the nucleic acid SEQ ID NO. 66274, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 66274 or polypeptide SEQ ID NO. 66275, respectively, or a homolog or a fragment thereof, is increased or generated, or if the activity polygalacturonase is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 59 to 149-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a polygalacturonase, or if the activity of the polypeptide Yjr153w, preferably represented by SEQ ID NO. 66275, or a homolog or fragment thereof, or if the activity of a polypeptide encoded by a nucleic acid molecule comprising the nucleic acid SEQ ID NO. 66274, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 66274 or polypeptide SEQ ID NO. 66275, respectively, or a homolog or a fragment thereof, is increased or generated, or if the activity polygalacturonase is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 34 to 51-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a YKL038W-protein, or if the activity of the polypeptide YKL038W, preferably represented by SEQ ID NO. 90603, or a homolog or fragment thereof, or if the activity of a polypeptide encoded by a nucleic acid molecule comprising the nucleic acid SEQ ID NO. 90602, preferably the coding region thereof, or a homolog or fragment thereof, e.g. derived from SACCHAROMYCES CEREVISIAE, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 90602 or polypeptide SEQ ID NO. 90603, respectively, or a homolog or a fragment thereof, is increased or generated, or if the activity YKL038W-protein is increased or generated in a non-human organism, like a microorganism or a plant cell, plant or part thereof, especially with plastidic localization, whereby the respective line discloses in column 7 alanine. For example, an increase of the alanine of at least 1 percent, particularly in a range of 20 to 45-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a YKL038W-protein, or if the activity of the polypeptide YKL038W, preferably represented by SEQ ID NO. 90603, or a homolog or fragment thereof, or if the activity of a polypeptide encoded by a nucleic acid molecule comprising the nucleic acid SEQ ID NO. 90602, preferably the coding region thereof, or a homolog or fragment thereof, e.g. derived from SACCHAROMYCES CEREVISIAE, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 90602 or polypeptide SEQ ID NO. 90603, respectively, or a homolog or a fragment thereof, is increased or generated, or if the activity YKL038W-protein is increased or generated in a non-human organism, like a microorganism or a plant cell, plant or part thereof, especially with plastidic localization, whereby the respective line discloses in column 7 aspartate. For example, an increase of the aspartate of at least 1 percent, particularly in a range of 59 to 125-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a YKL038W-protein, or if the activity of the polypeptide YKL038W, preferably represented by SEQ ID NO. 90603, or a homolog or fragment thereof, or if the activity of a polypeptide encoded by a nucleic acid molecule comprising the nucleic acid SEQ ID NO. 90602, preferably the coding region thereof, or a homolog or fragment thereof, e.g. derived from SACCHAROMYCES CEREVISIAE, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 90602 or polypeptide SEQ ID NO. 90603, respectively, or a homolog or a fragment thereof, is increased or generated, or if the activity YKL038W-protein is increased or generated in a non-human organism, like a microorganism or a plant cell, plant or part thereof, especially with plastidic localization, whereby the respective line discloses in column 7 serine. For example, an increase of the serine of at least 1 percent, particularly in a range of 27 to 57-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a aspartate aminotransferase, or if the activity of the polypeptide Ylr027c, preferably represented by SEQ ID NO. 90616, or a homolog or fragment thereof, or if the activity of a polypeptide encoded by a nucleic acid molecule comprising the nucleic acid SEQ ID NO. 90615, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 90615 or polypeptide SEQ ID NO. 90616, respectively, or a homolog or a fragment thereof, is increased or generated, or if the activity aspartate aminotransferase is increased or generated in a non-human organism, like a microorganism or a plant cell, plant or part thereof, especially with non-targeted localization, whereby the respective line discloses in column 7 glycine. For example, an increase of the glycine of at least 1 percent, particularly in a range of 41 to 69-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a thioredoxin, or if the activity of the polypeptide Ylr043c, preferably represented by SEQ ID NO. 91046, or a homolog or fragment thereof, or if the activity of a polypeptide encoded by a nucleic acid molecule comprising the nucleic acid SEQ ID NO. 91045, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 91045 or polypeptide SEQ ID NO. 91046, respectively, or a homolog or a fragment thereof, is increased or generated, or if the activity thioredoxin is increased or generated in a non-human organism, like a microorganism or a plant cell, plant or part thereof, especially with plastidic localization, whereby the respective line discloses in column 7 serine. For example, an increase of the serine of at least 1 percent, particularly in a range of 29 to 110-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a thioredoxin, or if the activity of the polypeptide Ylr043c, preferably represented by SEQ ID NO. 91046, or a homolog or fragment thereof, or if the activity of a polypeptide encoded by a nucleic acid molecule comprising the nucleic acid SEQ ID NO. 91045, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 91045 or polypeptide SEQ ID NO. 91046, respectively, or a homolog or a fragment thereof, is increased or generated, or if the activity thioredoxin is increased or generated in a non-human organism, like a microorganism or a plant cell, plant or part thereof, especially with plastidic localization, whereby the respective line discloses in column 7 tyrosine. For example, an increase of the tyrosine of at least 1 percent, particularly in a range of 40 to 94-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a sterol 24-C-methyltransferase, or if the activity of the polypeptide Yml008c, preferably represented by SEQ ID NO. 91660, or a homolog or fragment thereof, or if the activity of a polypeptide encoded by a nucleic acid molecule comprising the nucleic acid SEQ ID NO. 91659, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 91659 or polypeptide SEQ ID NO. 91660, respectively, or a homolog or a fragment thereof, is increased or generated, or if the activity sterol 24-C-methyltransferase is increased or generated in a non-human organism, like a microorganism or a plant cell, plant or part thereof, especially with plastidic localization, whereby the respective line discloses in column 7 alanine. For example, an increase of the alanine of at least 1 percent, particularly in a range of 21 to 52-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a sterol 24-C-methyltransferase, or if the activity of the polypeptide Yml008c, preferably represented by SEQ ID NO. 91660, or a homolog or fragment thereof, or if the activity of a polypeptide encoded by a nucleic acid molecule comprising the nucleic acid SEQ ID NO. 91659, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 91659 or polypeptide SEQ ID NO. 91660, respectively, or a homolog or a fragment thereof, is increased or generated, or if the activity sterol 24-C-methyltransferase is increased or generated in a non-human organism, like a microorganism or a plant cell, plant or part thereof, especially with plastidic localization, whereby the respective line discloses in column 7 glycine. For example, an increase of the glycine of at least 1 percent, particularly in a range of 40 to 96-percent is conferred as compared to a corresponding non-transformed wild type non-human organism.

Accordingly, in one embodiment, an increase of homoserine in a non-human organism, as compared to a corresponding non-transformed wild type non-human organism, is conferred in the process of the invention, if the activity of a polypeptide showing the activity of a sterol 24-C-methyltransferase, or if the activity of the polypeptide Yml008c, preferably represented by SEQ ID NO. 91660, or a homolog or fragment thereof, or if the activity of a polypeptide encoded by a nucleic acid molecule comprising the nucleic acid SEQ ID NO. 91659, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 91659 or polypeptide SEQ ID NO. 91660, respectively, or a homolog or a fragment thereof, is increased or generated, or if the activity sterol 24-C-methyltransferase is increased or generated in a non-human organism, like a microorganism or a plant cell, plant or part thereof, especially with plastidic localization, whereby the respective line discloses in column 7 homoserine. For example, an increase of the homoserine of at least 1 percent, particularly in a range of 30 to 46-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a sterol 24-C-methyltransferase, or if the activity of the polypeptide Yml008c, preferably represented by SEQ ID NO. 91660, or a homolog or fragment thereof, or if the activity of a polypeptide encoded by a nucleic acid molecule comprising the nucleic acid SEQ ID NO. 91659, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 91659 or polypeptide SEQ ID NO. 91660, respectively, or a homolog or a fragment thereof, is increased or generated, or if the activity sterol 24-C-methyltransferase is increased or generated in a non-human organism, like a microorganism or a plant cell, plant or part thereof, especially with plastidic localization, whereby the respective line discloses in column 7 5-oxoproline. For example, an increase of the 5-oxoproline of at least 1 percent, particularly in a range of 32 to 56-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a yml083c-protein, or if the activity of the polypeptide Yml083c, preferably represented by SEQ ID NO. 91782, or a homolog or fragment thereof, or if the activity of a polypeptide encoded by a nucleic acid molecule comprising the nucleic acid SEQ ID NO. 91781, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 91781 or polypeptide SEQ ID NO. 91782, respectively, or a homolog or a fragment thereof, is increased or generated, or if the activity yml083c-protein is increased or generated in a non-human organism, like a microorganism or a plant cell, plant or part thereof, especially with plastidic localization, whereby the respective line discloses in column 7 alanine. For example, an increase of the alanine of at least 1 percent, particularly in a range of 22 to 31-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a phosphate transporter, or if the activity of the polypeptide Yml123c, preferably represented by SEQ ID NO. 91786, or a homolog or fragment thereof, or if the activity of a polypeptide encoded by a nucleic acid molecule comprising the nucleic acid SEQ ID NO. 91785, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 91785 or polypeptide SEQ ID NO. 91786, respectively, or a homolog or a fragment thereof, is increased or generated, or if the activity phosphate transporter is increased or generated in a non-human organism, like a microorganism or a plant cell, plant or part thereof, especially with plastidic localization, whereby the respective line discloses in column 7 phenylalanine. For example, an increase of the phenylalanine of at least 1 percent, particularly in a range of 32 to 57-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a phosphate transporter, or if the activity of the polypeptide Yml123c, preferably represented by SEQ ID NO. 91786, or a homolog or fragment thereof, or if the activity of a polypeptide encoded by a nucleic acid molecule comprising the nucleic acid SEQ ID NO. 91785, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 91785 or polypeptide SEQ ID NO. 91786, respectively, or a homolog or a fragment thereof, is increased or generated, or if the activity phosphate transporter is increased or generated in a non-human organism, like a microorganism or a plant cell, plant or part thereof, especially with plastidic localization, whereby the respective line discloses in column 7 tyrosine. For example, an increase of the tyrosine of at least 1 percent, particularly in a range of 44 to 66-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a ymr013c-protein, or if the activity of the polypeptide Ymr013c, preferably represented by SEQ ID NO. 91959, or a homolog or fragment thereof, or if the activity of a polypeptide encoded by a nucleic acid molecule comprising the nucleic acid SEQ ID NO. 91958, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 91958 or polypeptide SEQ ID NO. 91959, respectively, or a homolog or a fragment thereof, is increased or generated, or if the activity ymr013c-protein is increased or generated in a non-human organism, like a microorganism or a plant cell, plant or part thereof, especially with plastidic localization, whereby the respective line discloses in column 7 tyrosine. For example, an increase of the tyrosine of at least 1 percent, particularly in a range of 41 to 93-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a purine biosynthesis protein, or if the activity of the polypeptide Ymr120c, preferably represented by SEQ ID NO. 91974, or a homolog or fragment thereof, or if the activity of a polypeptide encoded by a nucleic acid molecule comprising the nucleic acid SEQ ID NO. 91973, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 91973 or polypeptide SEQ ID NO. 91974, respectively, or a homolog or a fragment thereof, is increased or generated, or if the activity purine biosynthesis protein is increased or generated in a non-human organism, like a microorganism or a plant cell, plant or part thereof, especially with plastidic localization, whereby the respective line discloses in column 7 glycine. For example, an increase of the glycine of at least 1 percent, particularly in a range of 37 to 39-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a mitochondrial inner membrane protease subunit, or if the activity of the polypeptide Ymr150c, preferably represented by SEQ ID NO. 92275, or a homolog or fragment thereof, or if the activity of a polypeptide encoded by a nucleic acid molecule comprising the nucleic acid SEQ ID NO. 92274, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 92274 or polypeptide SEQ ID NO. 92275, respectively, or a homolog or a fragment thereof, is increased or generated, or if the activity mitochondrial inner membrane protease subunit is increased or generated in a non-human organism, like a microorganism or a plant cell, plant or part thereof, especially with plastidic localization, whereby the respective line discloses in column 7 phenylalanine. For example, an increase of the phenylalanine of at least 1 percent, particularly in a range of 25 to 52-percent is conferred as compared to a corresponding non-transformed wild type non-human organism.

Accordingly, in one embodiment, an increase of homoserine in a non-human organism, as compared to a corresponding non-transformed wild type non-human organism, is conferred in the process of the invention, if the activity of a polypeptide showing the activity of a YNL086W-protein, or if the activity of the polypeptide Yn1086w, preferably represented by SEQ ID NO. 92326, or a homolog or fragment thereof, or if the activity of a polypeptide encoded by a nucleic acid molecule comprising the nucleic acid SEQ ID NO. 92325, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 92325 or polypeptide SEQ ID NO. 92326, respectively, or a homolog or a fragment thereof, is increased or generated, or if the activity YNL086W-protein is increased or generated in a non-human organism, like a microorganism or a plant cell, plant or part thereof, especially with plastidic localization, whereby the respective line discloses in column 7 homoserine. For example, an increase of the homoserine of at least 1 percent, particularly in a range of 24 to 37-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a amino acid transporter, or if the activity of the polypeptide Ynl101w, preferably represented by SEQ ID NO. 92340, or a homolog or fragment thereof, or if the activity of a polypeptide encoded by a nucleic acid molecule comprising the nucleic acid SEQ ID NO. 92339, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 92339 or polypeptide SEQ ID NO. 92340, respectively, or a homolog or a fragment thereof, is increased or generated, or if the activity amino acid transporter is increased or generated in a non-human organism, like a microorganism or a plant cell, plant or part thereof, especially with plastidic localization, whereby the respective line discloses in column 7 tyrosine. For example, an increase of the tyrosine of at least 1 percent, particularly in a range of 33 to 43-percent is conferred as compared to a corresponding non-transformed wild type non-human organism.

Accordingly, in one embodiment, an increase of homoserine in a non-human organism, as compared to a corresponding non-transformed wild type non-human organism, is conferred in the process of the invention, if the activity of a polypeptide showing the activity of a homoserine O-acetyltransferase, or if the activity of the polypeptide Ynl277w, preferably represented by SEQ ID NO. 92392, or a homolog or fragment thereof, or if the activity of a polypeptide encoded by a nucleic acid molecule comprising the nucleic acid SEQ ID NO. 92391, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 92391 or polypeptide SEQ ID NO. 92392, respectively, or a homolog or a fragment thereof, is increased or generated, or if the activity homoserine O-acetyltransferase is increased or generated in a non-human organism, like a microorganism or a plant cell, plant or part thereof, especially with plastidic localization, whereby the respective line discloses in column 7 homoserine. For example, an increase of the homoserine of at least 1 percent, particularly in a range of 214 to 1243-percent is conferred as compared to a corresponding non-transformed wild type non-human organism.

Accordingly, in one embodiment, an increase of alanine in a non-human organism, as compared to a corresponding non-transformed wild type non-human organism, is conferred in the process of the invention, if the activity of a polypeptide showing the activity of a metal ion transporter, or if the activity of the polypeptide Yol130w, preferably represented by SEQ ID NO. 92511, or a homolog or fragment thereof, or if the activity of a polypeptide encoded by a nucleic acid molecule comprising the nucleic acid SEQ ID NO. 92510, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 92510 or polypeptide SEQ ID NO. 92511, respectively, or a homolog or a fragment thereof, is increased or generated, or if the activity metal ion transporter is increased or generated in a non-human organism, like a microorganism or a plant cell, plant or part thereof, especially with plastidic localization, whereby the respective line discloses in column 7 alanine. For example, an increase of the alanine of at least 1 percent, particularly in a range of 21 to 33-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a yol160w-protein, or if the activity of the polypeptide Yol160w, preferably represented by SEQ ID NO. 15180, or a homolog or fragment thereof, or if the activity of a polypeptide encoded by a nucleic acid molecule comprising the nucleic acid SEQ ID NO. 15179, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 15179 or polypeptide SEQ ID NO. 15180, respectively, or a homolog or a fragment thereof, is increased or generated, or if the activity yol160w-protein is increased or generated in a non-human organism, like a microorganism or a plant cell, plant or part thereof, especially with plastidic localization, whereby the respective line discloses in column 7 aspartate. For example, an increase of the aspartate of at least 1 percent, particularly in a range of 57 to 62-percent is conferred as compared to a corresponding non-transformed wild type non-human organism.

Accordingly, in one embodiment, an increase of citrulline in a non-human organism, as compared to a corresponding non-transformed wild type non-human organism, is conferred in the process of the invention, if the activity of a polypeptide showing the activity of a protein kinase, or if the activity of the polypeptide Yor233w, preferably represented by SEQ ID NO. 67969, or a homolog or fragment thereof, or if the activity of a polypeptide encoded by a nucleic acid molecule comprising the nucleic acid SEQ ID NO. 67968, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 67968 or polypeptide SEQ ID NO. 67969, respectively, or a homolog or a fragment thereof, is increased or generated, or if the activity protein kinase is increased or generated in a non-human organism, like a microorganism or a plant cell, plant or part thereof, especially with non-targeted localization, whereby the respective line discloses in column 7 citrulline. For example, an increase of the citrulline of at least 1 percent, particularly in a range of 36 to 92-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a histone acetyltransferase, or if the activity of the polypeptide Yor244w, preferably represented by SEQ ID NO. 92541, or a homolog or fragment thereof, or if the activity of a polypeptide encoded by a nucleic acid molecule comprising the nucleic acid SEQ ID NO. 92540, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 92540 or polypeptide SEQ ID NO. 92541, respectively, or a homolog or a fragment thereof, is increased or generated, or if the activity histone acetyltransferase is increased or generated in a non-human organism, like a microorganism or a plant cell, plant or part thereof, especially with plastidic localization, whereby the respective line discloses in column 7 phenylalanine. For example, an increase of the phenylalanine of at least 1 percent, particularly in a range of 44 to 423-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a histone acetyltransferase, or if the activity of the polypeptide Yor244w, preferably represented by SEQ ID NO. 92541, or a homolog or fragment thereof, or if the activity of a polypeptide encoded by a nucleic acid molecule comprising the nucleic acid SEQ ID NO. 92540, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 92540 or polypeptide SEQ ID NO. 92541, respectively, or a homolog or a fragment thereof, is increased or generated, or if the activity histone acetyltransferase is increased or generated in a non-human organism, like a microorganism or a plant cell, plant or part thereof, especially with plastidic localization, whereby the respective line discloses in column 7 tyrosine. For example, an increase of the tyrosine of at least 1 percent, particularly in a range of 53 to 85-percent is conferred as compared to a corresponding non-transformed wild type non-human organism.

Accordingly, in one embodiment, an increase of homoserine in a non-human organism, as compared to a corresponding non-transformed wild type non-human organism, is conferred in the process of the invention, if the activity of a polypeptide showing the activity of a diacylglycerol O-acyltransferase, or if the activity of the polypeptide Yor245c, preferably represented by SEQ ID NO. 92617, or a homolog or fragment thereof, or if the activity of a polypeptide encoded by a nucleic acid molecule comprising the nucleic acid SEQ ID NO. 92616, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 92616 or polypeptide SEQ ID NO. 92617, respectively, or a homolog or a fragment thereof, is increased or generated, or if the activity diacylglycerol O-acyltransferase is increased or generated in a non-human organism, like a microorganism or a plant cell, plant or part thereof, especially with non-targeted localization, whereby the respective line discloses in column 7 homoserine. For example, an increase of the homoserine of at least 1 percent, particularly in a range of 29 to 61-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a diacylglycerol O-acyltransferase, or if the activity of the polypeptide Yor245c, preferably represented by SEQ ID NO. 92617, or a homolog or fragment thereof, or if the activity of a polypeptide encoded by a nucleic acid molecule comprising the nucleic acid SEQ ID NO. 92616, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 92616 or polypeptide SEQ ID NO. 92617, respectively, or a homolog or a fragment thereof, is increased or generated, or if the activity diacylglycerol O-acyltransferase is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 24 to 82-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a diacylglycerol O-acyltransferase, or if the activity of the polypeptide Yor245c, preferably represented by SEQ ID NO. 92617, or a homolog or fragment thereof, or if the activity of a polypeptide encoded by a nucleic acid molecule comprising the nucleic acid SEQ ID NO. 92616, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 92616 or polypeptide SEQ ID NO. 92617, respectively, or a homolog or a fragment thereof, is increased or generated, or if the activity diacylglycerol O-acyltransferase is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 35 to 145-percent is conferred as compared to a corresponding non-transformed wild type non-human organism.

Accordingly, in one embodiment, an increase of glycine in a non-human organism, as compared to a corresponding non-transformed wild type non-human organism, is conferred in the process of the invention, if the activity of a polypeptide showing the activity of a acetyl-CoA acetyltransferase, or if the activity of the polypeptide Ypl028w, preferably represented by SEQ ID NO. 92669, or a homolog or fragment thereof, or if the activity of a polypeptide encoded by a nucleic acid molecule comprising the nucleic acid SEQ ID NO. 92668, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 92668 or polypeptide SEQ ID NO. 92669, respectively, or a homolog or a fragment thereof, is increased or generated, or if the activity acetyl-CoA acetyltransferase is increased or generated in a non-human organism, like a microorganism or a plant cell, plant or part thereof, especially with non-targeted localization, whereby the respective line discloses in column 7 glycine. For example, an increase of the glycine of at least 1 percent, particularly in a range of 43 to 82-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a citrate synthase, or if the activity of the polypeptide Ypr001w, preferably represented by SEQ ID NO. 93092, or a homolog or fragment thereof, or if the activity of a polypeptide encoded by a nucleic acid molecule comprising the nucleic acid SEQ ID NO. 93091, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 93091 or polypeptide SEQ ID NO. 93092, respectively, or a homolog or a fragment thereof, is increased or generated, or if the activity citrate synthase is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 23 to 31-percent is conferred as compared to a corresponding non-transformed wild type non-human organism.

Accordingly, in one embodiment, an increase of aspartate in a non-human organism, as compared to a corresponding non-transformed wild type non-human organism, is conferred in the process of the invention, if the activity of a polypeptide showing the activity of a ammonium transporter, or if the activity of the polypeptide Ypr138c, preferably represented by SEQ ID NO. 93219, or a homolog or fragment thereof, or if the activity of a polypeptide encoded by a nucleic acid molecule comprising the nucleic acid SEQ ID NO. 93218, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 93218 or polypeptide SEQ ID NO. 93219, respectively, or a homolog or a fragment thereof, is increased or generated, or if the activity ammonium transporter is increased or generated in a non-human organism, like a microorganism or a plant cell, plant or part thereof, especially with non-targeted localization, whereby the respective line discloses in column 7 aspartate. For example, an increase of the aspartate of at least 1 percent, particularly in a range of 42 to 217-percent is conferred as compared to a corresponding non-transformed wild type non-human organism.

Accordingly, in one embodiment, an increase of phenylalanine in a non-human organism, as compared to a corresponding non-transformed wild type non-human organism, is conferred in the process of the invention, if the activity of a polypeptide showing the activity of a Zm_{—}4842_BE510522-protein, or if the activity of the polypeptide Zm_{—}4842_BE510522, preferably represented by SEQ ID NO. 68414, or a homolog or fragment thereof, or if the activity of a polypeptide encoded by a nucleic acid molecule comprising the nucleic acid SEQ ID NO. 68413, preferably the coding region thereof, or a homolog or fragment thereof, e.g. derived from Zea mays, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 68413 or polypeptide SEQ ID NO. 68414, respectively, or a homolog or a fragment thereof, is increased or generated, or if the activity Zm_{—}4842_BE510522-protein is increased or generated in a non-human organism, like a microorganism or a plant cell, plant or part thereof, especially with non-targeted localization, whereby the respective line discloses in column 7 phenylalanine. For example, an increase of the phenylalanine of at least 1 percent, particularly in a range of 29 to 242-percent is conferred as compared to a corresponding non-transformed wild type non-human organism.

Accordingly, in one embodiment, an increase of serine in a non-human organism, as compared to a corresponding non-transformed wild type non-human organism, is conferred in the process of the invention, if the activity of a polypeptide showing the activity of a Zm_{—}4842_BE510522protein, or if the activity of the polypeptide Zm_{—}4842_BE510522, preferably represented by SEQ ID NO. 68414, or a homolog or fragment thereof, or if the activity of a polypeptide encoded by a nucleic acid molecule comprising the nucleic acid SEQ ID NO. 68413, preferably the coding region thereof, or a homolog or fragment thereof, e.g. derived from Zea mays, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 68413 or polypeptide SEQ ID NO. 68414, respectively, or a homolog or a fragment thereof, is increased or generated, or if the activity Zm_{—}4842_BE510522-protein is increased or generated in a non-human organism, like a microorganism or a plant cell, plant or part thereof, especially with non-targeted localization, whereby the respective line discloses in column 7 serine. For example, an increase of the serine of at least 1 percent, particularly in a range of 28 to 95-percent is conferred as compared to a corresponding non-transformed wild type non-human organism.

Accordingly, in one embodiment, an increase of tyrosine in a non-human organism, as compared to a corresponding non-transformed wild type non-human organism, is conferred in the process of the invention, if the activity of a polypeptide showing the activity of a Zm_{—}4842_BE510522-protein, or if the activity of the polypeptide Zm_{—}4842_BE510522, preferably represented by SEQ ID NO. 68414, or a homolog or fragment thereof, or if the activity of a polypeptide encoded by a nucleic acid molecule comprising the nucleic acid SEQ ID NO. 68413, preferably the coding region thereof, or a homolog or fragment thereof, e.g. derived from Zea mays, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 68413 or polypeptide SEQ ID NO. 68414, respectively, or a homolog or a fragment thereof, is increased or generated, or if the activity Zm_{—}4842_BE510522-protein is increased or generated in a non-human organism, like a microorganism or a plant cell, plant or part thereof, especially with non-targeted localization, whereby the respective line discloses in column 7 tyrosine. For example, an increase of the tyrosine of at least 1 percent, particularly in a range of 67 to 154-percent is conferred as compared to a corresponding non-transformed wild type non-human organism.

Accordingly, in one embodiment, an increase of 5-oxoproline in a non-human organism, as compared to a corresponding non-transformed wild type non-human organism, is conferred in the process of the invention, if the activity of a polypeptide showing the activity of a Zm_{—}4842_BE510522-protein, or if the activity of the polypeptide Zm_{—}4842_BE510522, preferably represented by SEQ ID NO. 68414, or a homolog or fragment thereof, or if the activity of a polypeptide encoded by a nucleic acid molecule comprising the nucleic acid SEQ ID NO. 68413, preferably the coding region thereof, or a homolog or fragment thereof, e.g. derived from Zea mays, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 3, column 5 or 8 in the respective same line as the nucleic acid molecule SEQ ID NO. 68413 or polypeptide SEQ ID NO. 68414, respectively, or a homolog or a fragment thereof, is increased or generated, or if the activity Zm_{—}4842_BE510522-protein is increased or generated in a non-human organism, like a microorganism or a plant cell, plant or part thereof, especially with non-targeted localization, whereby the respective line discloses in column 7 5-oxoproline. For example, an increase of the 5-oxoproline of at least 1 percent, particularly in a range of 33 to 173-percent is conferred as compared to a corresponding non-transformed wild type non-human organism.

[0096.1.1.3] to [0103.1.1.3] for the disclosure of these paragraphs see [0096.1.1.1] to [0103.1.1.1] above.

In this paragraph “A” means “Goffeau et al., Science 274 (5287), 546 (1996)”, “B” means “Blattner et al., Science 277 (5331), 1453 (1997)”, “C” means “Nakamura et al., Nucleic Acids Res. 28(1), 72 (2000)”, “D” means “deposition of the sequences to the EMBL/GenBank/DDBJ databases under “Sequencing of the draft genome assembly of Azotobacter vinelandii AvOP”. in June 2005 by Copeland et al.”, “E” means “Henne et al., Nat. Biotechnol. 22 (5), 547 (2004)”, “F” means “Kaul et al., Nature 408 (6814), 796 (2000)”, and “G” means “Matsumoto et al., Nature 436 (7052), 793 (2005)”.

The nucleic acid sequence of 47266012_SOYBEAN from Glycine max, e.g. as shown in the respective line in column 5 of Table I, application no. 3, is unpublished. And the activity of the gene product thereof is the activity of 47266012-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “47266012-protein”, especially from Glycine max or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said 47266012_SOYBEAN, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said 47266012_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said 47266012_SOYBEAN, or a functional equivalent or a homolog thereof as depicted in column 8 of Table II, application no. 3, preferably a homolog or functional equivalent as depicted in column 8 of Table II B, application no. 3, and being depicted in the same respective line as said 47266012_SOYBEAN, and preferably the activity is increased non-targeted

Accordingly, in one embodiment, the molecule which activity is to be increased in the process of the invention is the gene product with an activity as a “47266012-protein”, preferably it is the molecule of section (a) or (b) of this paragraph.

In particular, it was observed that in plants, especially in Arabidopsis thaliana, increasing or generating the activity of a gene product non-targeted with the activity of a “47266012-protein”, preferably being encoded by a gene comprising the nucleic acid sequence SEQ ID NO. 15187, preferably the coding region thereof, conferred the production of or the increase in aspartate compared with the wild type control.

The nucleic acid sequence of 47266012_SOYBEAN from Glycine max, e.g. as shown in the respective line in column 5 of Table I, application no. 3, is unpublished. And the activity of the gene product thereof is the activity of 47266012-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “47266012-protein”, especially from Glycine max or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said 47266012_SOYBEAN, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said 47266012_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said 47266012_SOYBEAN, or a functional equivalent or a homolog thereof as depicted in column 8 of Table II, application no. 3, preferably a homolog or functional equivalent as depicted in column 8 of Table II B, application no. 3, and being depicted in the same respective line as said 47266012_SOYBEAN, and preferably the activity is increased non-targeted.

Accordingly, in one embodiment, the molecule which activity is to be increased in the process of the invention is the gene product with an activity as a “47266012-protein”, preferably it is the molecule of section (a) or (b) of this paragraph.

In particular, it was observed that in plants, especially in Arabidopsis thaliana, increasing or generating the activity of a gene product non-targeted with the activity of a “47266012-protein”, preferably being encoded by a gene comprising the nucleic acid sequence SEQ ID NO. 15187, preferably the coding region thereof, conferred the production of or the increase in tyrosine compared with the wild type control.

The nucleic acid sequence of 47266012_SOYBEAN from Glycine max, e.g. as shown in the respective line in column 5 of Table I, application no. 3, is unpublished. And the activity of the gene product thereof is the activity of 47266012-protein.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “47266012-protein”, especially from Glycine max or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said 47266012_SOYBEAN, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said 47266012_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said 47266012_SOYBEAN, or a functional equivalent or a homolog thereof as depicted in column 8 of Table II, application no. 3, preferably a homolog or functional equivalent as depicted in column 8 of Table II B, application no. 3, and being depicted in the same respective line as said 47266012_SOYBEAN, and preferably the activity is increased non-targeted.

Accordingly, in one embodiment, the molecule which activity is to be increased in the process of the invention is the gene product with an activity as a “47266012-protein”, preferably it is the molecule of section (a) or (b) of this paragraph.

In particular, it was observed that in plants, especially in Arabidopsis thaliana, increasing or generating the activity of a gene product non-targeted with the activity of a “47266012-protein”, preferably being encoded by a gene comprising the nucleic acid sequence SEQ ID NO. 15187, preferably the coding region thereof, conferred the production of or the increase in 5-oxoproline compared with the wild type control.

The nucleic acid sequence of 49747384_SOYBEAN from Glycine max, e.g. as shown in the respective line in column 5 of Table I, application no. 3, is unpublished. And the activity of the gene product thereof is the activity of 49747384_SOYBEAN-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “49747384_SOYBEAN-protein”, especially from Glycine max or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said 49747384_SOYBEAN, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said 49747384_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said 49747384_SOYBEAN, or a functional equivalent or a homolog thereof as depicted in col 1 0 umn 8 of Table II, application no. 3, preferably a homolog or functional equivalent as depicted in column 8 of Table II B, application no. 3, and being depicted in the same respective line as said 49747384_SOYBEAN, and preferably the activity is increased non-targeted.

Accordingly, in one embodiment, the molecule which activity is to be increased in the process of the invention is the gene product with an activity as a “49747384_SOYBEAN-protein”, preferably it is the molecule of section (a) or (b) of this paragraph.

In particular, it was observed that in plants, especially in Arabidopsis thaliana, increasing or generating the activity of a gene product non-targeted with the activity of a “49747384_SOYBEAN-protein”, preferably being encoded by a gene comprising the nucleic acid sequence SEQ ID NO. 69, preferably the coding region thereof, conferred the production of or the increase in aspartate compared with the wild type control.

The nucleic acid sequence of 49747384_SOYBEAN from Glycine max, e.g. as shown in the respective line in column 5 of Table I, application no. 3, is unpublished. And the activity of the gene product thereof is the activity of 49747384_SOYBEAN-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “49747384_SOYBEAN-protein”, especially from Glycine max or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said 49747384_SOYBEAN, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said 49747384_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said 49747384_SOYBEAN, or a functional equivalent or a homolog thereof as depicted in column 8 of Table II, application no. 3, preferably a homolog or functional equivalent as depicted in column 8 of Table II B, application no. 3, and being depicted in the same respective line as said 49747384_SOYBEAN, and preferably the activity is increased non-targeted

Accordingly, in one embodiment, the molecule which activity is to be increased in the process of the invention is the gene product with an activity as a “49747384_SOYBEAN-protein”, preferably it is the molecule of section (a) or (b) of this paragraph.

In particular, it was observed that in plants, especially in Arabidopsis thaliana, increasing or generating the activity of a gene product non-targeted with the activity of a “49747384_SOYBEAN-protein”, preferably being encoded by a gene comprising the nucleic acid sequence SEQ ID NO. 69, preferably the coding region thereof, conferred the production of or the increase in tyrosine compared with the wild type control.

The nucleic acid sequence of 49747384_SOYBEAN from Glycine max, e.g. as shown in the respective line in column 5 of Table I, application no. 3, is unpublished. And the activity of the gene product thereof is the activity of 49747384_SOYBEAN-protein.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “49747384_SOYBEAN-protein”, especially from Glycine max or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said 49747384_SOYBEAN, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said 49747384_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said 49747384_SOYBEAN, or a functional equivalent or a homolog thereof as depicted in column 8 of Table II, application no. 3, preferably a homolog or functional equivalent as depicted in column 8 of Table II B, application no. 3, and being depicted in the same respective line as said 49747384_SOYBEAN, and preferably the activity is increased non-targeted.

Accordingly, in one embodiment, the molecule which activity is to be increased in the process of the invention is the gene product with an activity as a “49747384_SOYBEAN-protein”, preferably it is the molecule of section (a) or (b) of this paragraph.

In particular, it was observed that in plants, especially in Arabidopsis thaliana, increasing or generating the activity of a gene product non-targeted with the activity of a “49747384_SOYBEAN-protein”, preferably being encoded by a gene comprising the nucleic acid sequence SEQ ID NO. 69, preferably the coding region thereof, conferred the production of or the increase in 5-oxoproline compared with the wild type control.

The nucleic acid sequence of 51340801_CANOLA from Brassica napus, e.g. as shown in the respective line in column 5 of Table I, application no. 3, is unpublished. And the activity of the gene product thereof is the activity of pyruvate kinase.

Accordingly, in one embodiment, the process of the present invention for producing alanine in a non-human organism, like a microorganism or a plant or a part thereof, comprises increasing or generating the activity of a gene product with the activity of a gene product conferring the activity of “pyruvate kinase”, especially from Brassica napus or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said 51340801_CANOLA, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said 51340801_CANOLA, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said 51340801_CANOLA, or a functional equivalent or a homolog thereof as depicted in column 8 of Table II, application no. 3, preferably a homolog or functional equivalent as depicted in column 8 of Table II B, application no. 3, and being depicted in the same respective line as said 51340801_CANOLA, and preferably the activity is increased plastidic.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 15532, preferably the coding region thereof, conferred the production of or the increase in alanine compared with the wild type control.

The nucleic acid sequence of 51340801_CANOLA from Brassica napus, e.g. as shown in the respective line in column 5 of Table I, application no. 3, is unpublished. And the activity of the gene product thereof is the activity of pyruvate kinase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “pyruvate kinase”, especially from Brassica napus or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said 51340801_CANOLA, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said 51340801_CANOLA, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said 51340801_CANOLA, or a functional equivalent or a homolog thereof as depicted in column 8 of Table II, application no. 3, preferably a homolog or functional equivalent as depicted in column 8 of Table II B, application no. 3, and being depicted in the same respective line as said 51340801_CANOLA, and preferably the activity is increased plastidic.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 15532, preferably the coding region thereof, conferred the production of or the increase in phenylalanine compared with the wild type control.

The nucleic acid sequence of 51340801_CANOLA from Brassica napus, e.g. as shown in the respective line in column 5 of Table I, application no. 3, is unpublished. And the activity of the gene product thereof is the activity of pyruvate kinase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “pyruvate kinase”, especially from Brassica napus or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said 51340801_CANOLA, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said 51340801_CANOLA, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said 51340801_CANOLA, or a functional equivalent or a homolog thereof as depicted in column 8 of Table II, application no. 3, preferably a homolog or functional equivalent as depicted in column 8 of Table II B, application no. 3, and being depicted in the same respective line as said 51340801_CANOLA, and preferably the activity is increased plastidic.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 15532, preferably the coding region thereof, conferred the production of or the increase in tyrosine compared with the wild type control.

The nucleic acid sequence of 59547452_SOYBEAN from Glycine max, e.g. as shown in the respective line in column 5 of Table I, application no. 3, is unpublished. And the activity of the gene product thereof is the activity of acetyltransferase.

Accordingly, in one embodiment, the process of the present invention for producing citrulline in a non-human organism, like a microorganism or a plant or a part thereof, comprises increasing or generating the activity of a gene product with the activity of a gene product conferring the activity of “acetyltransferase”, especially from Glycine max or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said 59547452_SOYBEAN, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said 59547452_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said 59547452_SOYBEAN, or a functional equivalent or a homolog thereof as depicted in column 8 of Table II, application no. 3, preferably a homolog or functional equivalent as depicted in column 8 of Table II B, application no. 3, and being depicted in the same respective line as said 59547452_SOYBEAN, and preferably the activity is increased non-targeted.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 16155, preferably the coding region thereof, conferred the production of or the increase in citrulline compared with the wild type control.

The nucleic acid sequence of 59554615_SOYBEAN from Glycine max, e.g. as shown in the respective line in column 5 of Table I, application no. 3, is unpublished. And the activity of the gene product thereof is the activity of pyruvate kinase.

Accordingly, in one embodiment, the process of the present invention for producing asparagine in a non-human organism, like a microorganism or a plant or a part thereof, comprises increasing or generating the activity of a gene product with the activity of a gene product conferring the activity of “pyruvate kinase”, especially from Glycine max or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said 59554615_SOYBEAN, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said 59554615_SOYBEAN, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said 59554615_SOYBEAN, or a functional equivalent or a homolog thereof as depicted in column 8 of Table II, application no. 3, preferably a homolog or functional equivalent as depicted in column 8 of Table II B, application no. 3, and being depicted in the same respective line as said 59554615_SOYBEAN, and preferably the activity is increased plastidic.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 16263, preferably the coding region thereof, conferred the production of or the increase in asparagine compared with the wild type control.

The nucleic acid sequence of 59554615_SOYBEAN from Glycine max, e.g. as shown in the respective line in column 5 of Table I, application no. 3, is unpublished. And the activity of the gene product thereof is the activity of pyruvate kinase.

Accordingly, in one embodiment, the process of the present invention for producing serine in a non-human organism, like a microorganism or a plant or a part thereof, comprises increasing or generating the activity of a gene product with the activity of a gene product conferring the activity of “pyruvate kinase”, especially from Glycine max or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said 59554615_SOYBEAN, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said 59554615_SOYBEAN, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said 59554615_SOYBEAN, or a functional equivalent or a homolog thereof as depicted in column 8 of Table II, application no. 3, preferably a homolog or functional equivalent as depicted in column 8 of Table II B, application no. 3, and being depicted in the same respective line as said 59554615_SOYBEAN, and preferably the activity is increased plastidic.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 16263, preferably the coding region thereof, conferred the production of or the increase in serine compared with the wild type control.

The nucleic acid sequence of 59582753_SOYBEAN from Glycine max, e.g. as shown in the respective line in column 5 of Table I, application no. 3, is unpublished. And the activity of the gene product thereof is the activity of glucose-6-phosphate 1-dehydrogenase.

Accordingly, in one embodiment, the process of the present invention for producing alanine in a non-human organism, like a microorganism or a plant or a part thereof, comprises increasing or generating the activity of a gene product with the activity of a gene product conferring the activity of “glucose-6-phosphate 1-dehydrogenase”, especially from Glycine max or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said 59582753_SOYBEAN, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said 59582753_SOYBEAN, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said 59582753_SOYBEAN, or a functional equivalent or a homolog thereof as depicted in column 8 of Table II, application no. 3, preferably a homolog or functional equivalent as depicted in column 8 of Table II B, application no. 3, and being depicted in the same respective line as said 59582753_SOYBEAN, and preferably the activity is increased plastidic.

Accordingly, in one embodiment, the molecule which activity is to be increased in the process of the invention is the gene product with an activity as a “glucose-6-phosphate 1-dehydrogenase”, preferably it is the molecule of section (a) or (b) of this paragraph.

In particular, it was observed that in plants, especially in Arabidopsis thaliana, increasing or generating the activity of a gene product plastidic with the activity of a “glucose-6-phosphate 1-dehydrogenase”, preferably being encoded by a gene comprising the nucleic acid sequence SEQ ID NO. 16883, preferably the coding region thereof, conferred the production of or the increase in alanine compared with the wild type control.

The nucleic acid sequence of 59582753_SOYBEAN from Glycine max, e.g. as shown in the respective line in column 5 of Table I, application no. 3, is unpublished. And the activity of the gene product thereof is the activity of glucose-6-phosphate 1-dehydrogenase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “glucose-6-phosphate 1-dehydrogenase”, especially from Glycine max or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said 59582753_SOYBEAN, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said 59582753_SOYBEAN, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said 59582753_SOYBEAN, or a functional equivalent or a homolog thereof as depicted in column 8 of Table II, application no. 3, preferably a homolog or functional equivalent as depicted in column 8 of Table II B, application no. 3, and being depicted in the same respective line as said 59582753_SOYBEAN, and preferably the activity is increased plastidic.

Accordingly, in one embodiment, the molecule which activity is to be increased in the process of the invention is the gene product with an activity as a “glucose-6-phosphate 1-dehydrogenase”, preferably it is the molecule of section (a) or (b) of this paragraph.

In particular, it was observed that in plants, especially in Arabidopsis thaliana, increasing or generating the activity of a gene product plastidic with the activity of a “glucose-6-phosphate 1-dehydrogenase”, preferably being encoded by a gene comprising the nucleic acid sequence SEQ ID NO. 16883, preferably the coding region thereof, conferred the production of or the increase in phenylalanine compared with the wild type control.

The nucleic acid sequence of 59582753_SOYBEAN from Glycine max, e.g. as shown in the respective line in column 5 of Table I, application no. 3, is unpublished. And the activity of the gene product thereof is the activity of glucose-6-phosphate 1-dehydrogenase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “glucose-6-phosphate 1-dehydrogenase”, especially from Glycine max or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said 59582753_SOYBEAN, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said 59582753_SOYBEAN, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said 59582753_SOYBEAN, or a functional equivalent or a homolog thereof as depicted in column 8 of Table II, application no. 3, preferably a homolog or functional equivalent as depicted in column 8 of Table II B, application no. 3, and being depicted in the same respective line as said 59582753_SOYBEAN, and preferably the activity is increased plastidic.

Accordingly, in one embodiment, the molecule which activity is to be increased in the process of the invention is the gene product with an activity as a “glucose-6-phosphate 1-dehydrogenase”, preferably it is the molecule of section (a) or (b) of this paragraph.

In particular, it was observed that in plants, especially in Arabidopsis thaliana, increasing or generating the activity of a gene product plastidic with the activity of a “glucose-6-phosphate 1-dehydrogenase”, preferably being encoded by a gene comprising the nucleic acid sequence SEQ ID NO. 16883, preferably the coding region thereof, conferred the production of or the increase in tyrosine compared with the wild type control.

The nucleic acid sequence of AAC43185 from Arabidopsis thaliana, e.g. as shown in the respective line in column 5 of Table I, application no. 3, has been published in F. And the activity of the gene product thereof is the activity of amino acid acetyltransferase.

Accordingly, in one embodiment, the process of the present invention for producing alanine in a non-human organism, like a microorganism or a plant or a part thereof, comprises increasing or generating the activity of a gene product with the activity of a gene product conferring the activity of “amino acid acetyltransferase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AAC43185, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AAC43185, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AAC43185, or a functional equivalent or a homolog thereof as depicted in column 8 of Table II, application no. 3, preferably a homolog or functional equivalent as depicted in column 8 of Table II B, application no. 3, and being depicted in the same respective line as said AAC43185, and preferably the activity is increased plastidic.

Accordingly, in one embodiment, the molecule which activity is to be increased in the process of the invention is the gene product with an activity as a “amino acid acetyltransferase”, preferably it is the molecule of section (a) or (b) of this paragraph.

In particular, it was observed that in plants, especially in Arabidopsis thaliana, increasing or generating the activity of a gene product plastidic with the activity of a “amino acid acetyltransferase”, preferably being encoded by a gene comprising the nucleic acid sequence SEQ ID NO. 17356, preferably the coding region thereof, conferred the production of or the increase in alanine compared with the wild type control.

The nucleic acid sequence of AAC43185 from Arabidopsis thaliana, e.g. as shown in the respective line in column 5 of Table I, application no. 3, has been published in F. And the activity of the gene product thereof is the activity of amino acid acetyltransferase.

Accordingly, in one embodiment, the process of the present invention for producing citrulline in a non-human organism, like a microorganism or a plant or a part thereof, comprises increasing or generating the activity of a gene product with the activity of a gene product conferring the activity of “amino acid acetyltransferase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AAC43185, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AAC43185, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AAC43185, or a functional equivalent or a homolog thereof as depicted in column 8 of Table II, application no. 3, preferably a homolog or functional equivalent as depicted in column 8 of Table II B, application no. 3, and being depicted in the same respective line as said AAC43185, and preferably the activity is increased plastidic.

Accordingly, in one embodiment, the molecule which activity is to be increased in the process of the invention is the gene product with an activity as a “amino acid acetyltransferase”, preferably it is the molecule of section (a) or (b) of this paragraph.

In particular, it was observed that in plants, especially in Arabidopsis thaliana, increasing or generating the activity of a gene product plastidic with the activity of a “amino acid acetyltransferase”, preferably being encoded by a gene comprising the nucleic acid sequence SEQ ID NO. 17356, preferably the coding region thereof, conferred the production of or the increase in citrulline compared with the wild type control.

The nucleic acid sequence of AAC43185 from Arabidopsis thaliana, e.g. as shown in the respective line in column 5 of Table I, application no. 3, has been published in F. And the activity of the gene product thereof is the activity of amino acid acetyltransferase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “amino acid acetyltransferase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AAC43185, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AAC43185, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AAC43185, or a functional equivalent or a homolog thereof as depicted in column 8 of Table II, application no. 3, preferably a homolog or functional equivalent as depicted in column 8 of Table II B, application no. 3, and being depicted in the same respective line as said AAC43185, and preferably the activity is increased plastidic.

Accordingly, in one embodiment, the molecule which activity is to be increased in the process of the invention is the gene product with an activity as a “amino acid acetyltransferase”, preferably it is the molecule of section (a) or (b) of this paragraph.

In particular, it was observed that in plants, especially in Arabidopsis thaliana, increasing or generating the activity of a gene product plastidic with the activity of a “amino acid acetyltransferase”, preferably being encoded by a gene comprising the nucleic acid sequence SEQ ID NO. 17356, preferably the coding region thereof, conferred the production of or the increase in phenylalanine compared with the wild type control.

The nucleic acid sequence of At1g07430 from Arabidopsis thaliana, e.g. as shown in the respective line in column 5 of Table I, application no. 3, has been published in F. And the activity of the gene product thereof is the activity of protein phosphatase.

Accordingly, in one embodiment, the process of the present invention for producing citrulline in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g07430, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g07430, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g07430, or a functional equivalent or a homolog thereof as depicted in column 8 of Table II, application no. 3, preferably a homolog or functional equivalent as depicted in column 8 of Table II B, application no. 3, and being depicted in the same respective line as said At1g07430, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 17451, preferably the coding region thereof, conferred the production of or the increase in citrulline compared with the wild type control.

The nucleic acid sequence of At1g14490 from Arabidopsis thaliana, e.g. as shown in the respective line in column 5 of Table I, application no. 3, has been published in F. And the activity of the gene product thereof is the activity of DNA-binding protein.

Accordingly, in one embodiment, the process of the present invention for producing glycine in a non-human organism, like a microorganism or a plant or a part thereof, comprises increasing or generating the activity of a gene product with the activity of a gene product conferring the activity of “DNA-binding protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g14490, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g14490, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g14490, or a functional equivalent or a homolog thereof as depicted in column 8 of Table II, application no. 3, preferably a homolog or functional equivalent as depicted in column 8 of Table II B, application no. 3, and being depicted in the same respective line as said At1g14490, and preferably the activity is increased non-targeted.

Accordingly, in one embodiment, the molecule which activity is to be increased in the process of the invention is the gene product with an activity as a “DNA-binding protein”, preferably it is the molecule of section (a) or (b) of this paragraph.

In particular, it was observed that in plants, especially in Arabidopsis thaliana, increasing or generating the activity of a gene product non-targeted with the activity of a “DNA-binding protein”, preferably being encoded by a gene comprising the nucleic acid sequence SEQ ID NO. 385, preferably the coding region thereof, conferred the production of or the increase in glycine compared with the wild type control.

The nucleic acid sequence of At1g14490 from Arabidopsis thaliana, e.g. as shown in the respective line in column 5 of Table I, application no. 3, has been published in F. And the activity of the gene product thereof is the activity of DNA-binding protein.

Accordingly, in one embodiment, the process of the present invention for producing serine in a non-human organism, like a microorganism or a plant or a part thereof, comprises increasing or generating the activity of a gene product with the activity of a gene product conferring the activity of “DNA-binding protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g14490, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g14490, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g14490, or a functional equivalent or a homolog thereof as depicted in column 8 of Table II, application no. 3, preferably a homolog or functional equivalent as depicted in column 8 of Table II B, application no. 3, and being depicted in the same respective line as said At1g14490, and preferably the activity is increased non-targeted.

Accordingly, in one embodiment, the molecule which activity is to be increased in the process of the invention is the gene product with an activity as a “DNA-binding protein”, preferably it is the molecule of section (a) or (b) of this paragraph.

In particular, it was observed that in plants, especially in Arabidopsis thaliana, increasing or generating the activity of a gene product non-targeted with the activity of a “DNA-binding protein”, preferably being encoded by a gene comprising the nucleic acid sequence SEQ ID NO. 385, preferably the coding region thereof, conferred the production of or the increase in serine compared with the wild type control.

The nucleic acid sequence of At1g17440 from Arabidopsis thaliana, e.g. as shown in the respective line in column 5 of Table I, application no. 3, has been published in F. And the activity of the gene product thereof is the activity of At1g17440-protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “At1g17440-protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g17440, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g17440, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g17440, or a functional equivalent or a homolog thereof as depicted in column 8 of Table II, application no. 3, preferably a homolog or functional equivalent as depicted in column 8 of Table II B, application no. 3, and being depicted in the same respective line as said At1g17440, and preferably the activity is increased non-targeted.

Accordingly, in one embodiment, the molecule which activity is to be increased in the process of the invention is the gene product with an activity as a “At1g17440-protein”, preferably it is the molecule of section (a) or (b) of this paragraph.

In particular, it was observed that in plants, especially in Arabidopsis thaliana, increasing or generating the activity of a gene product non-targeted with the activity of a “At1g17440-protein”, preferably being encoded by a gene comprising the nucleic acid sequence SEQ ID NO. 17601, preferably the coding region thereof, conferred the production of or the increase in phenylalanine compared with the wild type control.

The nucleic acid sequence of At1g17440 from Arabidopsis thaliana, e.g. as shown in the respective line in column 5 of Table I, application no. 3, has been published in F. And the activity of the gene product thereof is the activity of At1g17440-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “At1g17440-protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g17440, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g17440, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g17440, or a functional equivalent or a homolog thereof as depicted in column 8 of Table II, application no. 3, preferably a homolog or functional equivalent as depicted in column 8 of Table II B, application no. 3, and being depicted in the same respective line as said At1g17440, and preferably the activity is increased non-targeted.

Accordingly, in one embodiment, the molecule which activity is to be increased in the process of the invention is the gene product with an activity as a “At1g17440-protein”, preferably it is the molecule of section (a) or (b) of this paragraph.

In particular, it was observed that in plants, especially in Arabidopsis thaliana, increasing or generating the activity of a gene product non-targeted with the activity of a “At1g17440-protein”, preferably being encoded by a gene comprising the nucleic acid sequence SEQ ID NO. 17601, preferably the coding region thereof, conferred the production of or the increase in tyrosine compared with the wild type control.

The nucleic acid sequence of At1g19800 from Arabidopsis thaliana, e.g. as shown in the respective line in column 5 of Table I, application no. 3, has been published in F. And the activity of the gene product thereof is the activity of At1g19800-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “At1g19800-protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g19800, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g19800, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g19800, or a functional equivalent or a homolog thereof as depicted in column 8 of Table II, application no. 3, preferably a homolog or functional equivalent as depicted in column 8 of Table II B, application no. 3, and being depicted in the same respective line as said At1g19800, and preferably the activity is increased non-targeted.

Accordingly, in one embodiment, the molecule which activity is to be increased in the process of the invention is the gene product with an activity as a “At1g19800-protein”, preferably it is the molecule of section (a) or (b) of this paragraph.

In particular, it was observed that in plants, especially in Arabidopsis thaliana, increasing or generating the activity of a gene product non-targeted with the activity of a “At1g19800-protein”, preferably being encoded by a gene comprising the nucleic acid sequence SEQ ID NO. 17637, preferably the coding region thereof, conferred the production of or the increase in aspartate compared with the wild type control.

The nucleic acid sequence of At1g19800 from Arabidopsis thaliana, e.g. as shown in the respective line in column 5 of Table I, application no. 3, has been published in F. And the activity of the gene product thereof is the activity of At1g19800-protein.

Accordingly, in one embodiment, the process of the present invention for producing citrulline in a non-human organism, like a microorganism or a plant or a part thereof, comprises increasing or generating the activity of a gene product with the activity of a gene product conferring the activity of “At1g19800-protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g19800, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g19800, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g19800, or a functional equivalent or a homolog thereof as depicted in column 8 of Table II, application no. 3, preferably a homolog or functional equivalent as depicted in column 8 of Table II B, application no. 3, and being depicted in the same respective line as said At1g19800, and preferably the activity is increased non-targeted.

Accordingly, in one embodiment, the molecule which activity is to be increased in the process of the invention is the gene product with an activity as a “At1g19800-protein”, preferably it is the molecule of section (a) or (b) of this paragraph.

In particular, it was observed that in plants, especially in Arabidopsis thaliana, increasing or generating the activity of a gene product non-targeted with the activity of a “At1g19800-protein”, preferably being encoded by a gene comprising the nucleic acid sequence SEQ ID NO. 17637, preferably the coding region thereof, conferred the production of or the increase in citrulline compared with the wild type control.

The nucleic acid sequence of At1g19800 from Arabidopsis thaliana, e.g. as shown in the respective line in column 5 of Table I, application no. 3, has been published in F. And the activity of the gene product thereof is the activity of At1g19800-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “At1g19800-protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g19800, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g19800, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g19800, or a functional equivalent or a homolog thereof as depicted in column 8 of Table II, application no. 3, preferably a homolog or functional equivalent as depicted in column 8 of Table II B, application no. 3, and being depicted in the same respective line as said At1g19800, and preferably the activity is increased non-targeted.

Accordingly, in one embodiment, the molecule which activity is to be increased in the process of the invention is the gene product with an activity as a “At1g19800-protein”, preferably it is the molecule of section (a) or (b) of this paragraph.

In particular, it was observed that in plants, especially in Arabidopsis thaliana, increasing or generating the activity of a gene product non-targeted with the activity of a “At1g19800-protein”, preferably being encoded by a gene comprising the nucleic acid sequence SEQ ID NO. 17637, preferably the coding region thereof, conferred the production of or the increase in tyrosine compared with the wild type control.

The nucleic acid sequence of At1g26830 from Arabidopsis thaliana, e.g. as shown in the respective line in column 5 of Table I, application no. 3, has been published in F. And the activity of the gene product thereof is the activity of cullin.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “cullin”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g26830, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g26830, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g26830, or a functional equivalent or a homolog thereof as depicted in column 8 of Table II, application no. 3, preferably a homolog or functional equivalent as depicted in column 8 of Table II B, application no. 3, and being depicted in the same respective line as said At1g26830, and preferably the activity is increased non-targeted.

Accordingly, in one embodiment, the molecule which activity is to be increased in the process of the invention is the gene product with an activity as a “cullin”, preferably it is the molecule of section (a) or (b) of this paragraph.

In particular, it was observed that in plants, especially in Arabidopsis thaliana, increasing or generating the activity of a gene product non-targeted with the activity of a “cullin”, preferably being encoded by a gene comprising the nucleic acid sequence SEQ ID NO. 17701, preferably the coding region thereof, conferred the production of or the increase in phenylalanine compared with the wild type control.

The nucleic acid sequence of At1g26830 from Arabidopsis thaliana, e.g. as shown in the respective line in column 5 of Table I, application no. 3, has been published in F. And the activity of the gene product thereof is the activity of cullin.

Accordingly, in one embodiment, the process of the present invention for producing tyrosine in a non-human organism, like a microorganism or a plant or a part thereof, comprises increasing or generating the activity of a gene product with the activity of a gene product conferring the activity of “cullin”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g26830, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g26830, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g26830, or a functional equivalent or a homolog thereof as depicted in column 8 of Table II, application no. 3, preferably a homolog or functional equivalent as depicted in column 8 of Table II B, application no. 3, and being depicted in the same respective line as said At1g26830, and preferably the activity is increased non-targeted.

Accordingly, in one embodiment, the molecule which activity is to be increased in the process of the invention is the gene product with an activity as a “cullin”, preferably it is the molecule of section (a) or (b) of this paragraph.

In particular, it was observed that in plants, especially in Arabidopsis thaliana, increasing or generating the activity of a gene product non-targeted with the activity of a “cullin”, preferably being encoded by a gene comprising the nucleic acid sequence SEQ ID NO. 17701, preferably the coding region thereof, conferred the production of or the increase in tyrosine compared with the wild type control.

The nucleic acid sequence of At1g31230 from Arabidopsis thaliana, e.g. as shown in the respective line in column 5 of Table I, application no. 3, has been published in F. And the activity of the gene product thereof is the activity of bifunctional aspartokinase/homoserine dehydrogenase.

Accordingly, in one embodiment, the process of the present invention for producing homoserine in a non-human organism, like a microorganism or a plant or a part thereof, comprises increasing or generating the activity of a gene product with the activity of a gene product conferring the activity of “bifunctional aspartokinase/homoserine dehydrogenase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g31230, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g31230, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g31230, or a functional equivalent or a homolog thereof as depicted in column 8 of Table II, application no. 3, preferably a homolog or functional equivalent as depicted in column 8 of Table II B, application no. 3, and being depicted in the same respective line as said At1g31230, and preferably the activity is increased non-targeted.

Accordingly, in one embodiment, the molecule which activity is to be increased in the process of the invention is the gene product with an activity as a “bifunctional aspartokinase/homoserine dehydrogenase”, preferably it is the molecule of section (a) or (b) of this paragraph.

In particular, it was observed that in plants, especially in Arabidopsis thaliana, increasing or generating the activity of a gene product non-targeted with the activity of a “bifunctional aspartokinase/homoserine dehydrogenase”, preferably being encoded by a gene comprising the nucleic acid sequence SEQ ID NO. 627, preferably the coding region thereof, conferred the production of or the increase in homoserine compared with the wild type control.

The nucleic acid sequence of At1g36730 from Arabidopsis thaliana, e.g. as shown in the respective line in column 5 of Table I, application no. 3, has been published in F. And the activity of the gene product thereof is the activity of eukaryotic translation initiation factor 5.

Accordingly, in one embodiment, the process of the present invention for producing aspartate in a non-human organism, like a microorganism or a plant or a part thereof, comprises increasing or generating the activity of a gene product with the activity of a gene product conferring the activity of “eukaryotic translation initiation factor 5”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g36730, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g36730, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g36730, or a functional equivalent or a homolog thereof as depicted in column 8 of Table II, application no. 3, preferably a homolog or functional equivalent as depicted in column 8 of Table II B, application no. 3, and being depicted in the same respective line as said At1g36730, and preferably the activity is increased non-targeted.

Accordingly, in one embodiment, the molecule which activity is to be increased in the process of the invention is the gene product with an activity as a “eukaryotic translation initiation factor 5”, preferably it is the molecule of section (a) or (b) of this paragraph.

In particular, it was observed that in plants, especially in Arabidopsis thaliana, increasing or generating the activity of a gene product non-targeted with the activity of a “eukaryotic translation initiation factor 5”, preferably being encoded by a gene comprising the nucleic acid sequence SEQ ID NO. 17968, preferably the coding region thereof, conferred the production of or the increase in aspartate compared with the wild type control.

The nucleic acid sequence of At1g36730 from Arabidopsis thaliana, e.g. as shown in the respective line in column 5 of Table I, application no. 3, has been published in F. And the activity of the gene product thereof is the activity of eukaryotic translation initiation factor 5.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “eukaryotic translation initiation factor 5”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g36730, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g36730, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g36730, or a functional equivalent or a homolog thereof as depicted in column 8 of Table II, application no. 3, preferably a homolog or functional equivalent as depicted in column 8 of Table II B, application no. 3, and being depicted in the same respective line as said At1g36730, and preferably the activity is increased non-targeted.

Accordingly, in one embodiment, the molecule which activity is to be increased in the process of the invention is the gene product with an activity as a “eukaryotic translation initiation factor 5”, preferably it is the molecule of section (a) or (b) of this paragraph.

In particular, it was observed that in plants, especially in Arabidopsis thaliana, increasing or generating the activity of a gene product non-targeted with the activity of a “eukaryotic translation initiation factor 5”, preferably being encoded by a gene comprising the nucleic acid sequence SEQ ID NO. 17968, preferably the coding region thereof, conferred the production of or the increase in phenylalanine compared with the wild type control.

The nucleic acid sequence of At1g36730 from Arabidopsis thaliana, e.g. as shown in the respective line in column 5 of Table I, application no. 3, has been published in F. And the activity of the gene product thereof is the activity of eukaryotic translation initiation factor 5.

Accordingly, in one embodiment, the process of the present invention for producing serine in a non-human organism, like a microorganism or a plant or a part thereof, comprises increasing or generating the activity of a gene product with the activity of a gene product conferring the activity of “eukaryotic translation initiation factor 5”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g36730, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g36730, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g36730, or a functional equivalent or a homolog thereof as depicted in column 8 of Table II, application no. 3, preferably a homolog or functional equivalent as depicted in column 8 of Table II B, application no. 3, and being depicted in the same respective line as said At1g36730, and preferably the activity is increased non-targeted.

Accordingly, in one embodiment, the molecule which activity is to be increased in the process of the invention is the gene product with an activity as a “eukaryotic translation initiation factor 5”, preferably it is the molecule of section (a) or (b) of this paragraph.

In particular, it was observed that in plants, especially in Arabidopsis thaliana, increasing or generating the activity of a gene product non-targeted with the activity of a “eukaryotic translation initiation factor 5”, preferably being encoded by a gene comprising the nucleic acid sequence SEQ ID NO. 17968, preferably the coding region thereof, conferred the production of or the increase in serine compared with the wild type control.

The nucleic acid sequence of At1g36730 from Arabidopsis thaliana, e.g. as shown in the respective line in column 5 of Table I, application no. 3, has been published in F. And the activity of the gene product thereof is the activity of eukaryotic translation initiation factor 5.

Accordingly, in one embodiment, the process of the present invention for producing tyrosine in a non-human organism, like a microorganism or a plant or a part thereof, comprises increasing or generating the activity of a gene product with the activity of a gene product conferring the activity of “eukaryotic translation initiation factor 5”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g36730, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g36730, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g36730, or a functional equivalent or a homolog thereof as depicted in column 8 of Table II, application no. 3, preferably a homolog or functional equivalent as depicted in column 8 of Table II B, application no. 3, and being depicted in the same respective line as said At1g36730, and preferably the activity is increased non-targeted.

Accordingly, in one embodiment, the molecule which activity is to be increased in the process of the invention is the gene product with an activity as a “eukaryotic translation initiation factor 5”, preferably it is the molecule of section (a) or (b) of this paragraph.

In particular, it was observed that in plants, especially in Arabidopsis thaliana, increasing or generating the activity of a gene product non-targeted with the activity of a “eukaryotic translation initiation factor 5”, preferably being encoded by a gene comprising the nucleic acid sequence SEQ ID NO. 17968, preferably the coding region thereof, conferred the production of or the increase in tyrosine compared with the wild type control.

The nucleic acid sequence of At1g36730 from Arabidopsis thaliana, e.g. as shown in the respective line in column 5 of Table I, application no. 3, has been published in F. And the activity of the gene product thereof is the activity of eukaryotic translation initiation factor 5.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “eukaryotic translation initiation factor 5”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g36730, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g36730, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g36730, or a functional equivalent or a homolog thereof as depicted in column 8 of Table II, application no. 3, preferably a homolog or functional equivalent as depicted in column 8 of Table II B, application no. 3, and being depicted in the same respective line as said At1g36730, and preferably the activity is increased non-targeted.

Accordingly, in one embodiment, the molecule which activity is to be increased in the process of the invention is the gene product with an activity as a “eukaryotic translation initiation factor 5”, preferably it is the molecule of section (a) or (b) of this paragraph.

In particular, it was observed that in plants, especially in Arabidopsis thaliana, increasing or generating the activity of a gene product non-targeted with the activity of a “eukaryotic translation initiation factor 5”, preferably being encoded by a gene comprising the nucleic acid sequence SEQ ID NO. 17968, preferably the coding region thereof, conferred the production of or the increase in 5-oxoproline compared with the wild type control.

The nucleic acid sequence of At1g43850 from Arabidopsis thaliana, e.g. as shown in the respective line in column 5 of Table I, application no. 3, has been published in F. And the activity of the gene product thereof is the activity of transcriptional regulator.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “transcriptional regulator”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g43850, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g43850, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g43850, or a functional equivalent or a homolog thereof as depicted in column 8 of Table II, application no. 3, preferably a homolog or functional equivalent as depicted in column 8 of Table II B, application no. 3, and being depicted in the same respective line as said At1g43850, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 18070, preferably the coding region thereof, conferred the production of or the increase in phenylalanine compared with the wild type control.

The nucleic acid sequence of At1g43850 from Arabidopsis thaliana, e.g. as shown in the respective line in column 5 of Table I, application no. 3, has been published in F. And the activity of the gene product thereof is the activity of transcriptional regulator.

Accordingly, in one embodiment, the process of the present invention for producing serine in a non-human organism, like a microorganism or a plant or a part thereof, comprises increasing or generating the activity of a gene product with the activity of a gene product conferring the activity of “transcriptional regulator”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g43850, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g43850, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g43850, or a functional equivalent or a homolog thereof as depicted in column 8 of Table II, application no. 3, preferably a homolog or functional equivalent as depicted in column 8 of Table II B, application no. 3, and being depicted in the same respective line as said At1g43850, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 18070, preferably the coding region thereof, conferred the production of or the increase in serine compared with the wild type control.

The nucleic acid sequence of At1g43850 from Arabidopsis thaliana, e.g. as shown in the respective line in column 5 of Table I, application no. 3, has been published in F. And the activity of the gene product thereof is the activity of transcriptional regulator.

Accordingly, in one embodiment, the process of the present invention for producing tyrosine in a non-human organism, like a microorganism or a plant or a part thereof, comprises increasing or generating the activity of a gene product with the activity of a gene product conferring the activity of “transcriptional regulator”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g43850, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g43850, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g43850, or a functional equivalent or a homolog thereof as depicted in column 8 of Table II, application no. 3, preferably a homolog or functional equivalent as depicted in column 8 of Table II B, application no. 3, and being depicted in the same respective line as said At1g43850, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 18070, preferably the coding region thereof, conferred the production of or the increase in tyrosine compared with the wild type control.

The nucleic acid sequence of At1g43850 from Arabidopsis thaliana, e.g. as shown in the respective line in column 5 of Table I, application no. 3, has been published in F. And the activity of the gene product thereof is the activity of transcriptional regulator.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “transcriptional regulator”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g43850, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g43850, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g43850, or a functional equivalent or a homolog thereof as depicted in column 8 of Table II, application no. 3, preferably a homolog or functional equivalent as depicted in column 8 of Table II B, application no. 3, and being depicted in the same respective line as said At1g43850, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 18070, preferably the coding region thereof, conferred the production of or the increase in 5-oxoproline compared with the wild type control.

The nucleic acid sequence of At1g48040 from Arabidopsis thaliana, e.g. as shown in the respective line in column 5 of Table I, application no. 3, has been published in F. And the activity of the gene product thereof is the activity of protein phosphatase.

Accordingly, in one embodiment, the process of the present invention for producing glycine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g48040, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g48040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g48040, or a functional equivalent or a homolog thereof as depicted in column 8 of Table II, application no. 3, preferably a homolog or functional equivalent as depicted in column 8 of Table II B, application no. 3, and being depicted in the same respective line as said At1g48040, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 812, preferably the coding region thereof, conferred the production of or the increase in glycine compared with the wild type control.

The nucleic acid sequence of At1g48260 from Arabidopsis thaliana, e.g. as shown in the respective line in column 5 of Table I, application no. 3, has been published in F. And the activity of the gene product thereof is the activity of protein kinase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g48260, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g48260, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g48260, or a functional equivalent or a homolog thereof as depicted in column 8 of Table II, application no. 3, preferably a homolog or functional equivalent as depicted in column 8 of Table II B, application no. 3, and being depicted in the same respective line as said At1g48260, and preferably the activity is increased non-targeted.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 18235, preferably the coding region thereof, conferred the production of or the increase in phenylalanine compared with the wild type control.

The nucleic acid sequence of At1g48260 from Arabidopsis thaliana, e.g. as shown in the respective line in column 5 of Table I, application no. 3, has been published in F. And the activity of the gene product thereof is the activity of protein kinase.

Accordingly, in one embodiment, the process of the present invention for producing serine in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g48260, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g48260, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g48260, or a functional equivalent or a homolog thereof as depicted in column 8 of Table II, application no. 3, preferably a homolog or functional equivalent as depicted in column 8 of Table II B, application no. 3, and being depicted in the same respective line as said At1g48260, and preferably the activity is increased non-targeted.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 18235, preferably the coding region thereof, conferred the production of or the increase in serine compared with the wild type control.

The nucleic acid sequence of At1g48260 from Arabidopsis thaliana, e.g. as shown in the respective line in column 5 of Table I, application no. 3, has been published in F. And the activity of the gene product thereof is the activity of protein kinase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g48260, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g48260, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g48260, or a functional equivalent or a homolog thereof as depicted in column 8 of Table II, application no. 3, preferably a homolog or functional equivalent as depicted in column 8 of Table II B, application no. 3, and being depicted in the same respective line as said At1g48260, and preferably the activity is increased non-targeted.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 18235, preferably the coding region thereof, conferred the production of or the increase in tyrosine compared with the wild type control.

The nucleic acid sequence of At1g61950 from Arabidopsis thaliana, e.g. as shown in the respective line in column 5 of Table I, application no. 3, has been published in F. And the activity of the gene product thereof is the activity of calcium-dependent protein kinase.

Accordingly, in one embodiment, the process of the present invention for producing aspartate in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g61950, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g61950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g61950, or a functional equivalent or a homolog thereof as depicted in column 8 of Table II, application no. 3, preferably a homolog or functional equivalent as depicted in column 8 of Table II B, application no. 3, and being depicted in the same respective line as said At1g61950, and preferably the activity is increased non-targeted.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 18869, preferably the coding region thereof, conferred the production of or the increase in aspartate compared with the wild type control.

The nucleic acid sequence of At1g61950 from Arabidopsis thaliana, e.g. as shown in the respective line in column 5 of Table I, application no. 3, has been published in F. And the activity of the gene product thereof is the activity of calcium-dependent protein kinase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g61950, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g61950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g61950, or a functional equivalent or a homolog thereof as depicted in column 8 of Table II, application no. 3, preferably a homolog or functional equivalent as depicted in column 8 of Table II B, application no. 3, and being depicted in the same respective line as said At1g61950, and preferably the activity is increased non-targeted.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 18869, preferably the coding region thereof, conferred the production of or the increase in phenylalanine compared with the wild type control.

The nucleic acid sequence of At1g72770 from Arabidopsis thaliana, e.g. as shown in the respective line in column 5 of Table I, application no. 3, has been published in F. And the activity of the gene product thereof is the activity of protein phosphatase.

Accordingly, in one embodiment, the process of the present invention for producing alanine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g72770, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g72770, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g72770, or a functional equivalent or a homolog thereof as depicted in column 8 of Table II, application no. 3, preferably a homolog or functional equivalent as depicted in column 8 of Table II B, application no. 3, and being depicted in the same respective line as said At1g72770, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19419, preferably the coding region thereof, conferred the production of or the increase in alanine compared with the wild type control.

The nucleic acid sequence of At1g72770 from Arabidopsis thaliana, e.g. as shown in the respective line in column 5 of Table I, application no. 3, has been published in F. And the activity of the gene product thereof is the activity of protein phosphatase.

Accordingly, in one embodiment, the process of the present invention for producing glycine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g72770, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g72770, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g72770, or a functional equivalent or a homolog thereof as depicted in column 8 of Table II, application no. 3, preferably a homolog or functional equivalent as depicted in column 8 of Table II B, application no. 3, and being depicted in the same respective line as said At1g72770, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19419, preferably the coding region thereof, conferred the production of or the increase in glycine compared with the wild type control.

The nucleic acid sequence of At1g72770 from Arabidopsis thaliana, e.g. as shown in the respective line in column 5 of Table I, application no. 3, has been published in F. And the activity of the gene product thereof is the activity of protein phosphatase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g72770, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g72770, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g72770, or a functional equivalent or a homolog thereof as depicted in column 8 of Table II, application no. 3, preferably a homolog or functional equivalent as depicted in column 8 of Table II B, application no. 3, and being depicted in the same respective line as said At1g72770, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19419, preferably the coding region thereof, conferred the production of or the increase in tyrosine compared with the wild type control.

The nucleic acid sequence of At1g72770 from Arabidopsis thaliana, e.g. as shown in the respective line in column 5 of Table I, application no. 3, has been published in F. And the activity of the gene product thereof is the activity of protein phosphatase.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At1g72770, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At1g72770, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At1g72770, or a functional equivalent or a homolog thereof as depicted in column 8 of Table II, application no. 3, preferably a homolog or functional equivalent as depicted in column 8 of Table II B, application no. 3, and being depicted in the same respective line as said At1g72770, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19419, preferably the coding region thereof, conferred the production of or the increase in 5-oxoproline compared with the wild type control.

The nucleic acid sequence of At2g13360 from Arabidopsis thaliana, e.g. as shown in the respective line in column 5 of Table I, application no. 3, has been published in F. And the activity of the gene product thereof is the activity of aminotransferase.

Accordingly, in one embodiment, the process of the present invention for producing alanine in a non-human organism, like a microorganism or a plant or a part thereof, comprises increasing or generating the activity of a gene product with the activity of a gene product conferring the activity of “aminotransferase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At2g13360, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At2g13360, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At2g13360, or a functional equivalent or a homolog thereof as depicted in column 8 of Table II, application no. 3, preferably a homolog or functional equivalent as depicted in column 8 of Table II B, application no. 3, and being depicted in the same respective line as said At2g13360, and preferably the activity is increased non-targeted.

Accordingly, in one embodiment, the molecule which activity is to be increased in the process of the invention is the gene product with an activity as a “aminotransferase”, preferably it is the molecule of section (a) or (b) of this paragraph.

In particular, it was observed that in plants, especially in Arabidopsis thaliana, increasing or generating the activity of a gene product non-targeted with the activity of a “aminotransferase”, preferably being encoded by a gene comprising the nucleic acid sequence SEQ ID NO. 68536, preferably the coding region thereof, conferred the production of or the increase in alanine compared with the wild type control.

The nucleic acid sequence of At2g17560 from Arabidopsis thaliana, e.g. as shown in the respective line in column 5 of Table I, application no. 3, has been published in F. And the activity of the gene product thereof is the activity of transcription factor.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At2g17560, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At2g17560, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At2g17560, or a functional equivalent or a homolog thereof as depicted in column 8 of Table II, application no. 3, preferably a homolog or functional equivalent as depicted in column 8 of Table II B, application no. 3, and being depicted in the same respective line as said At2g17560, and preferably the activity is increased non-targeted.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 19502, preferably the coding region thereof, conferred the production of or the increase in phenylalanine compared with the wild type control.

The nucleic acid sequence of At2g17560 from Arabidopsis thaliana, e.g. as shown in the respective line in column 5 of Table I, application no. 3, has been published in F. And the activity of the gene product thereof is the activity of transcription factor.

Accordingly, in one embodiment, the process of the present invention for producing tyrosine in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At2g17560, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At2g17560, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At2g17560, or a functional equivalent or a homolog thereof as depicted in column 8 of Table II, application no. 3, preferably a homolog or functional equivalent as depicted in column 8 of Table II B, application no. 3, and being depicted in the same respective line as said At2g17560, and preferably the activity is increased non-targeted.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 19502, preferably the coding region thereof, conferred the production of or the increase in tyrosine compared with the wild type control.

The nucleic acid sequence of At2g25070 from Arabidopsis thaliana, e.g. as shown in the respective line in column 5 of Table I, application no. 3, has been published in F. And the activity of the gene product thereof is the activity of protein phosphatase.

Accordingly, in one embodiment, the process of the present invention for producing aspartate in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At2g25070, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At2g25070, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At2g25070, or a functional equivalent or a homolog thereof as depicted in column 8 of Table II, application no. 3, preferably a homolog or functional equivalent as depicted in column 8 of Table II B, application no. 3, and being depicted in the same respective line as said At2g25070, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 1298, preferably the coding region thereof, conferred the production of or the increase in aspartate compared with the wild type control.

The nucleic acid sequence of At2g25070 from Arabidopsis thaliana, e.g. as shown in the respective line in column 5 of Table I, application no. 3, has been published in F. And the activity of the gene product thereof is the activity of protein phosphatase.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At2g25070, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At2g25070, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At2g25070, or a functional equivalent or a homolog thereof as depicted in column 8 of Table II, application no. 3, preferably a homolog or functional equivalent as depicted in column 8 of Table II B, application no. 3, and being depicted in the same respective line as said At2g25070, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 1298, preferably the coding region thereof, conferred the production of or the increase in 5-oxoproline compared with the wild type control.

The nucleic acid sequence of At2g28890 from Arabidopsis thaliana, e.g. as shown in the respective line in column 5 of Table I, application no. 3, has been published in F. And the activity of the gene product thereof is the activity of protein phosphatase.

Accordingly, in one embodiment, the process of the present invention for producing asparagine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At2g28890, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At2g28890, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At2g28890, or a functional equivalent or a homolog thereof as depicted in column 8 of Table II, application no. 3, preferably a homolog or functional equivalent as depicted in column 8 of Table II B, application no. 3, and being depicted in the same respective line as said At2g28890, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19874, preferably the coding region thereof, conferred the production of or the increase in asparagine compared with the wild type control.

The nucleic acid sequence of At2g28890 from Arabidopsis thaliana, e.g. as shown in the respective line in column 5 of Table I, application no. 3, has been published in F. And the activity of the gene product thereof is the activity of protein phosphatase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At2g28890, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At2g28890, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At2g28890, or a functional equivalent or a homolog thereof as depicted in column 8 of Table II, application no. 3, preferably a homolog or functional equivalent as depicted in column 8 of Table II B, application no. 3, and being depicted in the same respective line as said At2g28890, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19874, preferably the coding region thereof, conferred the production of or the increase in phenylalanine compared with the wild type control.

The nucleic acid sequence of At2g28890 from Arabidopsis thaliana, e.g. as shown in the respective line in column 5 of Table I, application no. 3, has been published in F. And the activity of the gene product thereof is the activity of protein phosphatase.

Accordingly, in one embodiment, the process of the present invention for producing serine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At2g28890, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At2g28890, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At2g28890, or a functional equivalent or a homolog thereof as depicted in column 8 of Table II, application no. 3, preferably a homolog or functional equivalent as depicted in column 8 of Table II B, application no. 3, and being depicted in the same respective line as said At2g28890, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19874, preferably the coding region thereof, conferred the production of or the increase in serine compared with the wild type control.

The nucleic acid sequence of At2g28890 from Arabidopsis thaliana, e.g. as shown in the respective line in column 5 of Table I, application no. 3, has been published in F. And the activity of the gene product thereof is the activity of protein phosphatase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At2g28890, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At2g28890, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At2g28890, or a functional equivalent or a homolog thereof as depicted in column 8 of Table II, application no. 3, preferably a homolog or functional equivalent as depicted in column 8 of Table II B, application no. 3, and being depicted in the same respective line as said At2g28890, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19874, preferably the coding region thereof, conferred the production of or the increase in tyrosine compared with the wild type control.

The nucleic acid sequence of At2g30360 from Arabidopsis thaliana, e.g. as shown in the respective line in column 5 of Table I, application no. 3, has been published in F. And the activity of the gene product thereof is the activity of CBL-interacting protein kinase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “CBL-interacting protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At2g30360, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At2g30360, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At2g30360, or a functional equivalent or a homolog thereof as depicted in column 8 of Table II, application no. 3, preferably a homolog or functional equivalent as depicted in column 8 of Table II B, application no. 3, and being depicted in the same respective line as said At2g30360, and preferably the activity is increased non-targeted.

Accordingly, in one embodiment, the molecule which activity is to be increased in the process of the invention is the gene product with an activity as a “CBL-interacting protein kinase”, preferably it is the molecule of section (a) or (b) of this paragraph.

In particular, it was observed that in plants, especially in Arabidopsis thaliana, increasing or generating the activity of a gene product non-targeted with the activity of a “CBL-interacting protein kinase”, preferably being encoded by a gene comprising the nucleic acid sequence SEQ ID NO. 19919, preferably the coding region thereof, conferred the production of or the increase in phenylalanine compared with the wild type control.

The nucleic acid sequence of At2g30360 from Arabidopsis thaliana, e.g. as shown in the respective line in column 5 of Table I, application no. 3, has been published in F. And the activity of the gene product thereof is the activity of CBL-interacting protein kinase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “CBL-interacting protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At2g30360, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At2g30360, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At2g30360, or a functional equivalent or a homolog thereof as depicted in column 8 of Table II, application no. 3, preferably a homolog or functional equivalent as depicted in column 8 of Table II B, application no. 3, and being depicted in the same respective line as said At2g30360, and preferably the activity is increased non-targeted.

Accordingly, in one embodiment, the molecule which activity is to be increased in the process of the invention is the gene product with an activity as a “CBL-interacting protein kinase”, preferably it is the molecule of section (a) or (b) of this paragraph.

In particular, it was observed that in plants, especially in Arabidopsis thaliana, increasing or generating the activity of a gene product non-targeted with the activity of a “CBL-interacting protein kinase”, preferably being encoded by a gene comprising the nucleic acid sequence SEQ ID NO. 19919, preferably the coding region thereof, conferred the production of or the increase in tyrosine compared with the wild type control.

The nucleic acid sequence of At2g30360 from Arabidopsis thaliana, e.g. as shown in the respective line in column 5 of Table I, application no. 3, has been published in F. And the activity of the gene product thereof is the activity of CBL-interacting protein kinase.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “CBL-interacting protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At2g30360, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At2g30360, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At2g30360, or a functional equivalent or a homolog thereof as depicted in column 8 of Table II, application no. 3, preferably a homolog or functional equivalent as depicted in column 8 of Table II B, application no. 3, and being depicted in the same respective line as said At2g30360, and preferably the activity is increased non-targeted.

Accordingly, in one embodiment, the molecule which activity is to be increased in the process of the invention is the gene product with an activity as a “CBL-interacting protein kinase”, preferably it is the molecule of section (a) or (b) of this paragraph.

In particular, it was observed that in plants, especially in Arabidopsis thaliana, increasing or generating the activity of a gene product non-targeted with the activity of a “CBL-interacting protein kinase”, preferably being encoded by a gene comprising the nucleic acid sequence SEQ ID NO. 19919, preferably the coding region thereof, conferred the production of or the increase in 5-oxoproline compared with the wild type control.

The nucleic acid sequence of At2g30540 from Arabidopsis thaliana, e.g. as shown in the respective line in column 5 of Table I, application no. 3, has been published in F. And the activity of the gene product thereof is the activity of glutaredoxin.

Accordingly, in one embodiment, the process of the present invention for producing serine in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At2g30540, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At2g30540, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At2g30540, or a functional equivalent or a homolog thereof as depicted in column 8 of Table II, application no. 3, preferably a homolog or functional equivalent as depicted in column 8 of Table II B, application no. 3, and being depicted in the same respective line as said At2g30540, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 20346, preferably the coding region thereof, conferred the production of or the increase in serine compared with the wild type control.

The nucleic acid sequence of At2g30540 from Arabidopsis thaliana, e.g. as shown in the respective line in column 5 of Table I, application no. 3, has been published in F. And the activity of the gene product thereof is the activity of glutaredoxin.

Accordingly, in one embodiment, the process of the present invention for producing tyrosine in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At2g30540, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At2g30540, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At2g30540, or a functional equivalent or a homolog thereof as depicted in column 8 of Table II, application no. 3, preferably a homolog or functional equivalent as depicted in column 8 of Table II B, application no. 3, and being depicted in the same respective line as said At2g30540, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 20346, preferably the coding region thereof, conferred the production of or the increase in tyrosine compared with the wild type control.

The nucleic acid sequence of At2g34180 from Arabidopsis thaliana, e.g. as shown in the respective line in column 5 of Table I, application no. 3, has been published in F. And the activity of the gene product thereof is the activity of CBL-interacting protein kinase.

Accordingly, in one embodiment, the process of the present invention for producing glycine in a non-human organism, like a microorganism or a plant or a part thereof, comprises increasing or generating the activity of a gene product with the activity of a gene product conferring the activity of “CBL-interacting protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At2g34180, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At2g34180, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At2g34180, or a functional equivalent or a homolog thereof as depicted in column 8 of Table II, application no. 3, preferably a homolog or functional equivalent as depicted in column 8 of Table II B, application no. 3, and being depicted in the same respective line as said At2g34180, and preferably the activity is increased non-targeted.

Accordingly, in one embodiment, the molecule which activity is to be increased in the process of the invention is the gene product with an activity as a “CBL-interacting protein kinase”, preferably it is the molecule of section (a) or (b) of this paragraph.

In particular, it was observed that in plants, especially in Arabidopsis thaliana, increasing or generating the activity of a gene product non-targeted with the activity of a “CBL-interacting protein kinase”, preferably being encoded by a gene comprising the nucleic acid sequence SEQ ID NO. 20578, preferably the coding region thereof, conferred the production of or the increase in glycine compared with the wild type control.

The nucleic acid sequence of At2g39800 from Arabidopsis thaliana, e.g. as shown in the respective line in column 5 of Table I, application no. 3, has been published in F. And the activity of the gene product thereof is the activity of delta-1-pyrroline 5-carboxylase synthetase.

Accordingly, in one embodiment, the process of the present invention for producing alanine in a non-human organism, like a microorganism or a plant or a part thereof, comprises increasing or generating the activity of a gene product with the activity of a gene product conferring the activity of “delta-1-pyrroline 5-carboxylase synthetase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At2g39800, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At2g39800, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At2g39800, or a functional equivalent or a homolog thereof as depicted in column 8 of Table II, application no. 3, preferably a homolog or functional equivalent as depicted in column 8 of Table II B, application no. 3, and being depicted in the same respective line as said At2g39800, and preferably the activity is increased non-targeted.

Accordingly, in one embodiment, the molecule which activity is to be increased in the process of the invention is the gene product with an activity as a “delta-1-pyrroline 5-carboxylase synthetase”, preferably it is the molecule of section (a) or (b) of this paragraph.

In particular, it was observed that in plants, especially in Arabidopsis thaliana, increasing or generating the activity of a gene product non-targeted with the activity of a “delta-1-pyrroline 5-carboxylase synthetase”, preferably being encoded by a gene comprising the nucleic acid sequence SEQ ID NO. 21008, preferably the coding region thereof, conferred the production of or the increase in alanine compared with the wild type control.

The nucleic acid sequence of At2g39800 from Arabidopsis thaliana, e.g. as shown in the respective line in column 5 of Table I, application no. 3, has been published in F. And the activity of the gene product thereof is the activity of delta-1-pyrroline 5-carboxylase synthetase.

Accordingly, in one embodiment, the process of the present invention for producing glycine in a non-human organism, like a microorganism or a plant or a part thereof, comprises increasing or generating the activity of a gene product with the activity of a gene product conferring the activity of “delta-1-pyrroline 5-carboxylase synthetase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At2g39800, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At2g39800, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At2g39800, or a functional equivalent or a homolog thereof as depicted in column 8 of Table II, application no. 3, preferably a homolog or functional equivalent as depicted in column 8 of Table II B, application no. 3, and being depicted in the same respective line as said At2g39800, and preferably the activity is increased non-targeted.

Accordingly, in one embodiment, the molecule which activity is to be increased in the process of the invention is the gene product with an activity as a “delta-1-pyrroline 5-carboxylase synthetase”, preferably it is the molecule of section (a) or (b) of this paragraph.

In particular, it was observed that in plants, especially in Arabidopsis thaliana, increasing or generating the activity of a gene product non-targeted with the activity of a “delta-1-pyrroline 5carboxylase synthetase”, preferably being encoded by a gene comprising the nucleic acid sequence SEQ ID NO. 21008, preferably the coding region thereof, conferred the production of or the increase in glycine compared with the wild type control.

The nucleic acid sequence of At2g47880 from Arabidopsis thaliana, e.g. as shown in the respective line in column 5 of Table I, application no. 3, has been published in F. And the activity of the gene product thereof is the activity of glutaredoxin.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At2g47880, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At2g47880, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At2g47880, or a functional equivalent or a homolog thereof as depicted in column 8 of Table II, application no. 3, preferably a homolog or functional equivalent as depicted in column 8 of Table II B, application no. 3, and being depicted in the same respective line as said At2g47880, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 21159, preferably the coding region thereof, conferred the production of or the increase in phenylalanine compared with the wild type control.

The nucleic acid sequence of At2g47880 from Arabidopsis thaliana, e.g. as shown in the respective line in column 5 of Table I, application no. 3, has been published in F. And the activity of the gene product thereof is the activity of glutaredoxin.

Accordingly, in one embodiment, the process of the present invention for producing tyrosine in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At2g47880, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At2g47880, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At2g47880, or a functional equivalent or a homolog thereof as depicted in column 8 of Table II, application no. 3, preferably a homolog or functional equivalent as depicted in column 8 of Table II B, application no. 3, and being depicted in the same respective line as said At2g47880, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 21159, preferably the coding region thereof, conferred the production of or the increase in tyrosine compared with the wild type control.

The nucleic acid sequence of At3g02990 from Arabidopsis thaliana, e.g. as shown in the respective line in column 5 of Table I, application no. 3, has been published in F. And the activity of the gene product thereof is the activity of heat shock transcription factor.

Accordingly, in one embodiment, the process of the present invention for producing tyrosine in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At3g02990, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At3g02990, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At3g02990, or a functional equivalent or a homolog thereof as depicted in column 8 of Table II, application no. 3, preferably a homolog or functional equivalent as depicted in column 8 of Table II B, application no. 3, and being depicted in the same respective line as said At3g02990, and preferably the activity is increased non-targeted.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 68657, preferably the coding region thereof, conferred the production of or the increase in tyrosine compared with the wild type control.

The nucleic acid sequence of At3g04050 from Arabidopsis thaliana, e.g. as shown in the respective line in column 5 of Table I, application no. 3, has been published in F. And the activity of the gene product thereof is the activity of pyruvate kinase.

Accordingly, in one embodiment, the process of the present invention for producing alanine in a non-human organism, like a microorganism or a plant or a part thereof, comprises increasing or generating the activity of a gene product with the activity of a gene product conferring the activity of “pyruvate kinase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At3g04050, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At3g04050, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At3g04050, or a functional equivalent or a homolog thereof as depicted in column 8 of Table II, application no. 3, preferably a homolog or functional equivalent as depicted in column 8 of Table II B, application no. 3, and being depicted in the same respective line as said At3g04050, and preferably the activity is increased plastidic.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 21497, preferably the coding region thereof, conferred the production of or the increase in alanine compared with the wild type control.

The nucleic acid sequence of At3g04050 from Arabidopsis thaliana, e.g. as shown in the respective line in column 5 of Table I, application no. 3, has been published in F. And the activity of the gene product thereof is the activity of pyruvate kinase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “pyruvate kinase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At3g04050, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At3g04050, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At3g04050, or a functional equivalent or a homolog thereof as depicted in column 8 of Table II, application no. 3, preferably a homolog or functional equivalent as depicted in column 8 of Table II B, application no. 3, and being depicted in the same respective line as said At3g04050, and preferably the activity is increased plastidic.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 21497, preferably the coding region thereof, conferred the production of or the increase in phenylalanine compared with the wild type control.

The nucleic acid sequence of At3g04050 from Arabidopsis thaliana, e.g. as shown in the respective line in column 5 of Table I, application no. 3, has been published in F. And the activity of the gene product thereof is the activity of pyruvate kinase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “pyruvate kinase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At3g04050, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At3g04050, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At3g04050, or a functional equivalent or a homolog thereof as depicted in column 8 of Table II, application no. 3, preferably a homolog or functional equivalent as depicted in column 8 of Table II B, application no. 3, and being depicted in the same respective line as said At3g04050, and preferably the activity is increased plastidic.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 21497, preferably the coding region thereof, conferred the production of or the increase in tyrosine compared with the wild type control.

The nucleic acid sequence of At3g04710 from Arabidopsis thaliana, e.g. as shown in the respective line in column 5 of Table I, application no. 3, has been published in F. And the activity of the gene product thereof is the activity of ankyrin repeat family protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “ankyrin repeat family protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At3g04710, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At3g04710, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At3g04710, or a functional equivalent or a homolog thereof as depicted in column 8 of Table II, application no. 3, preferably a homolog or functional equivalent as depicted in column 8 of Table II B, application no. 3, and being depicted in the same respective line as said At3g04710, and preferably the activity is increased non-targeted.

Accordingly, in one embodiment, the molecule which activity is to be increased in the process of the invention is the gene product with an activity as a “ankyrin repeat family protein”, preferably it is the molecule of section (a) or (b) of this paragraph.

In particular, it was observed that in plants, especially in Arabidopsis thaliana, increasing or generating the activity of a gene product non-targeted with the activity of a “ankyrin repeat family protein”, preferably being encoded by a gene comprising the nucleic acid sequence SEQ ID NO. 21902, preferably the coding region thereof, conferred the production of or the increase in phenylalanine compared with the wild type control.

The nucleic acid sequence of At3g06270 from Arabidopsis thaliana, e.g. as shown in the respective line in column 5 of Table I, application no. 3, has been published in F. And the activity of the gene product thereof is the activity of protein phosphatase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At3g06270, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At3g06270, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At3g06270, or a functional equivalent or a homolog thereof as depicted in column 8 of Table II, application no. 3, preferably a homolog or functional equivalent as depicted in column 8 of Table II B, application no. 3, and being depicted in the same respective line as said At3g06270, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 22015, preferably the coding region thereof, conferred the production of or the increase in tyrosine compared with the wild type control.

The nucleic acid sequence of At3g08710 from Arabidopsis thaliana, e.g. as shown in the respective line in column 5 of Table I, application no. 3, has been published in F. And the activity of the gene product thereof is the activity of thioredoxin family protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “thioredoxin family protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At3g08710, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At3g08710, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At3g08710, or a functional equivalent or a homolog thereof as depicted in column 8 of Table II, application no. 3, preferably a homolog or functional equivalent as depicted in column 8 of Table II B, application no. 3, and being depicted in the same respective line as said At3g08710, and preferably the activity is increased non-targeted.

Accordingly, in one embodiment, the molecule which activity is to be increased in the process of the invention is the gene product with an activity as a “thioredoxin family protein”, preferably it is the molecule of section (a) or (b) of this paragraph.

In particular, it was observed that in plants, especially in Arabidopsis thaliana, increasing or generating the activity of a gene product non-targeted with the activity of a “thioredoxin family protein”, preferably being encoded by a gene comprising the nucleic acid sequence SEQ ID NO. 22249, preferably the coding region thereof, conferred the production of or the increase in aspartate compared with the wild type control.

The nucleic acid sequence of At3g08710 from Arabidopsis thaliana, e.g. as shown in the respective line in column 5 of Table I, application no. 3, has been published in F. And the activity of the gene product thereof is the activity of thioredoxin family protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “thioredoxin family protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At3g08710, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At3g08710, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At3g08710, or a functional equivalent or a homolog thereof as depicted in column 8 of Table II, application no. 3, preferably a homolog or functional equivalent as depicted in column 8 of Table II B, application no. 3, and being depicted in the same respective line as said At3g08710, and preferably the activity is increased non-targeted.

Accordingly, in one embodiment, the molecule which activity is to be increased in the process of the invention is the gene product with an activity as a “thioredoxin family protein”, preferably it is the molecule of section (a) or (b) of this paragraph.

In particular, it was observed that in plants, especially in Arabidopsis thaliana, increasing or generating the activity of a gene product non-targeted with the activity of a “thioredoxin family protein”, preferably being encoded by a gene comprising the nucleic acid sequence SEQ ID NO. 22249, preferably the coding region thereof, conferred the production of or the increase in phenylalanine compared with the wild type control.

The nucleic acid sequence of At3g08710 from Arabidopsis thaliana, e.g. as shown in the respective line in column 5 of Table I, application no. 3, has been published in F. And the activity of the gene product thereof is the activity of thioredoxin family protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “thioredoxin family protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At3g08710, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At3g08710, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At3g08710, or a functional equivalent or a homolog thereof as depicted in column 8 of Table II, application no. 3, preferably a homolog or functional equivalent as depicted in column 8 of Table II B, application no. 3, and being depicted in the same respective line as said At3g08710, and preferably the activity is increased non-targeted.

Accordingly, in one embodiment, the molecule which activity is to be increased in the process of the invention is the gene product with an activity as a “thioredoxin family protein”, preferably it is the molecule of section (a) or (b) of this paragraph.

In particular, it was observed that in plants, especially in Arabidopsis thaliana, increasing or generating the activity of a gene product non-targeted with the activity of a “thioredoxin family protein”, preferably being encoded by a gene comprising the nucleic acid sequence SEQ ID NO. 22249, preferably the coding region thereof, conferred the production of or the increase in tyrosine compared with the wild type control.

The nucleic acid sequence of At3g11650 from Arabidopsis thaliana, e.g. as shown in the respective line in column 5 of Table I, application no. 3, has been published in F. And the activity of the gene product thereof is the activity of harpin-induced family protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “harpin-induced family protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At3g11650, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At3g11650, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At3g11650, or a functional equivalent or a homolog thereof as depicted in column 8 of Table II, application no. 3, preferably a homolog or functional equivalent as depicted in column 8 of Table II B, application no. 3, and being depicted in the same respective line as said At3g11650, and preferably the activity is increased non-targeted.

Accordingly, in one embodiment, the molecule which activity is to be increased in the process of the invention is the gene product with an activity as a “harpin-induced family protein”, preferably it is the molecule of section (a) or (b) of this paragraph.

In particular, it was observed that in plants, especially in Arabidopsis thaliana, increasing or generating the activity of a gene product non-targeted with the activity of a “harpin-induced family protein”, preferably being encoded by a gene comprising the nucleic acid sequence SEQ ID NO. 22611, preferably the coding region thereof, conferred the production of or the increase in phenylalanine compared with the wild type control.

The nucleic acid sequence of At3g11650 from Arabidopsis thaliana, e.g. as shown in the respective line in column 5 of Table I, application no. 3, has been published in F. And the activity of the gene product thereof is the activity of harpin-induced family protein.

Accordingly, in one embodiment, the process of the present invention for producing serine in a non-human organism, like a microorganism or a plant or a part thereof, comprises increasing or generating the activity of a gene product with the activity of a gene product conferring the activity of “harpin-induced family protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At3g11650, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At3g11650, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At3g11650, or a functional equivalent or a homolog thereof as depicted in column 8 of Table II, application no. 3, preferably a homolog or functional equivalent as depicted in column 8 of Table II B, application no. 3, and being depicted in the same respective line as said At3g11650, and preferably the activity is increased non-targeted.

Accordingly, in one embodiment, the molecule which activity is to be increased in the process of the invention is the gene product with an activity as a “harpin-induced family protein”, preferably it is the molecule of section (a) or (b) of this paragraph.

In particular, it was observed that in plants, especially in Arabidopsis thaliana, increasing or generating the activity of a gene product non-targeted with the activity of a “harpin-induced family protein”, preferably being encoded by a gene comprising the nucleic acid sequence SEQ ID NO. 22611, preferably the coding region thereof, conferred the production of or the increase in serine compared with the wild type control.

The nucleic acid sequence of At3g11650 from Arabidopsis thaliana, e.g. as shown in the respective line in column 5 of Table I, application no. 3, has been published in F. And the activity of the gene product thereof is the activity of harpin-induced family protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “harpin-induced family protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At3g11650, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At3g11650, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At3g11650, or a functional equivalent or a homolog thereof as depicted in column 8 of Table II, application no. 3, preferably a homolog or functional equivalent as depicted in column 8 of Table II B, application no. 3, and being depicted in the same respective line as said At3g11650, and preferably the activity is increased non-targeted.

Accordingly, in one embodiment, the molecule which activity is to be increased in the process of the invention is the gene product with an activity as a “harpin-induced family protein”, preferably it is the molecule of section (a) or (b) of this paragraph.

In particular, it was observed that in plants, especially in Arabidopsis thaliana, increasing or generating the activity of a gene product non-targeted with the activity of a “harpin-induced family protein”, preferably being encoded by a gene comprising the nucleic acid sequence SEQ ID NO. 22611, preferably the coding region thereof, conferred the production of or the increase in tyrosine compared with the wild type control.

The nucleic acid sequence of At3g18524 from Arabidopsis thaliana, e.g. as shown in the respective line in column 5 of Table I, application no. 3, has been published in F. And the activity of the gene product thereof is the activity of DNA mismatch repair protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “DNA mismatch repair protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At3g18524, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At3g18524, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At3g18524, or a functional equivalent or a homolog thereof as depicted in column 8 of Table II, application no. 3, preferably a homolog or functional equivalent as depicted in column 8 of Table II B, application no. 3, and being depicted in the same respective line as said At3g18524, and preferably the activity is increased non-targeted.

Accordingly, in one embodiment, the molecule which activity is to be increased in the process of the invention is the gene product with an activity as a “DNA mismatch repair protein”, preferably it is the molecule of section (a) or (b) of this paragraph.

In particular, it was observed that in plants, especially in Arabidopsis thaliana, increasing or generating the activity of a gene product non-targeted with the activity of a “DNA mismatch repair protein”, preferably being encoded by a gene comprising the nucleic acid sequence SEQ ID NO. 22832, preferably the coding region thereof, conferred the production of or the increase in phenylalanine compared with the wild type control.

The nucleic acid sequence of At3g23000 from Arabidopsis thaliana, e.g. as shown in the respective line in column 5 of Table I, application no. 3, has been published in F. And the activity of the gene product thereof is the activity of protein kinase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At3g23000, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At3g23000, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At3g23000, or a functional equivalent or a homolog thereof as depicted in column 8 of Table II, application no. 3, preferably a homolog or functional equivalent as depicted in column 8 of Table II B, application no. 3, and being depicted in the same respective line as said At3g23000, and preferably the activity is increased non-targeted.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 1815, preferably the coding region thereof, conferred the production of or the increase in phenylalanine compared with the wild type control.

The nucleic acid sequence of At3g23000 from Arabidopsis thaliana, e.g. as shown in the respective line in column 5 of Table I, application no. 3, has been published in F. And the activity of the gene product thereof is the activity of protein kinase.

Accordingly, in one embodiment, the process of the present invention for producing serine in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At3g23000, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At3g23000, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At3g23000, or a functional equivalent or a homolog thereof as depicted in column 8 of Table II, application no. 3, preferably a homolog or functional equivalent as depicted in column 8 of Table II B, application no. 3, and being depicted in the same respective line as said At3g23000, and preferably the activity is increased non-targeted.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 1815, preferably the coding region thereof, conferred the production of or the increase in serine compared with the wild type control.

The nucleic acid sequence of At3g23000 from Arabidopsis thaliana, e.g. as shown in the respective line in column 5 of Table I, application no. 3, has been published in F. And the activity of the gene product thereof is the activity of protein kinase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At3g23000, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At3g23000, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At3g23000, or a functional equivalent or a homolog thereof as depicted in column 8 of Table II, application no. 3, preferably a homolog or functional equivalent as depicted in column 8 of Table II B, application no. 3, and being depicted in the same respective line as said At3g23000, and preferably the activity is increased non-targeted.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 1815, preferably the coding region thereof, conferred the production of or the increase in tyrosine compared with the wild type control.

The nucleic acid sequence of At3g23000 from Arabidopsis thaliana, e.g. as shown in the respective line in column 5 of Table I, application no. 3, has been published in F. And the activity of the gene product thereof is the activity of protein kinase.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At3g23000, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At3g23000, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At3g23000, or a functional equivalent or a homolog thereof as depicted in column 8 of Table II, application no. 3, preferably a homolog or functional equivalent as depicted in column 8 of Table II B, application no. 3, and being depicted in the same respective line as said At3g23000, and preferably the activity is increased non-targeted.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 1815, preferably the coding region thereof, conferred the production of or the increase in 5-oxoproline compared with the wild type control.

The nucleic acid sequence of At3g26400 from Arabidopsis thaliana, e.g. as shown in the respective line in column 5 of Table I, application no. 3, has been published in F. And the activity of the gene product thereof is the activity of eukaryotic translation initiation factor.

Accordingly, in one embodiment, the process of the present invention for producing serine in a non-human organism, like a microorganism or a plant or a part thereof, comprises increasing or generating the activity of a gene product with the activity of a gene product conferring the activity of “eukaryotic translation initiation factor”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At3g26400, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At3g26400, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At3g26400, or a functional equivalent or a homolog thereof as depicted in column 8 of Table II, application no. 3, preferably a homolog or functional equivalent as depicted in column 8 of Table II B, application no. 3, and being depicted in the same respective line as said At3g26400, and preferably the activity is increased non-targeted.

Accordingly, in one embodiment, the molecule which activity is to be increased in the process of the invention is the gene product with an activity as a “eukaryotic translation initiation factor”, preferably it is the molecule of section (a) or (b) of this paragraph.

In particular, it was observed that in plants, especially in Arabidopsis thaliana, increasing or generating the activity of a gene product non-targeted with the activity of a “eukaryotic translation initiation factor”, preferably being encoded by a gene comprising the nucleic acid sequence SEQ ID NO. 68727, preferably the coding region thereof, conferred the production of or the increase in serine compared with the wild type control.

The nucleic acid sequence of At3g27300 from Arabidopsis thaliana, e.g. as shown in the respective line in column 5 of Table I, application no. 3, has been published in F. And the activity of the gene product thereof is the activity of glucose-6-phosphate 1-dehydrogenase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “glucose-6-phosphate 1-dehydrogenase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At3g27300, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At3g27300, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At3g27300, or a functional equivalent or a homolog thereof as depicted in column 8 of Table II, application no. 3, preferably a homolog or functional equivalent as depicted in column 8 of Table II B, application no. 3, and being depicted in the same respective line as said At3g27300, and preferably the activity is increased non-targeted.

Accordingly, in one embodiment, the molecule which activity is to be increased in the process of the invention is the gene product with an activity as a “glucose-6-phosphate 1-dehydrogenase”, preferably it is the molecule of section (a) or (b) of this paragraph.

In particular, it was observed that in plants, especially in Arabidopsis thaliana, increasing or generating the activity of a gene product non-targeted with the activity of a “glucose-6-phosphate 1-dehydrogenase”, preferably being encoded by a gene comprising the nucleic acid sequence SEQ ID NO. 23002, preferably the coding region thereof, conferred the production of or the increase in tyrosine compared with the wild type control.

The nucleic acid sequence of At3g27540 from Arabidopsis thaliana, e.g. as shown in the respective line in column 5 of Table I, application no. 3, has been published in F. And the activity of the gene product thereof is the activity of glycosyl transferase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “glycosyl transferase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At3g27540, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At3g27540, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At3g27540, or a functional equivalent or a homolog thereof as depicted in column 8 of Table II, application no. 3, preferably a homolog or functional equivalent as depicted in column 8 of Table II B, application no. 3, and being depicted in the same respective line as said At3g27540, and preferably the activity is increased non-targeted.

Accordingly, in one embodiment, the molecule which activity is to be increased in the process of the invention is the gene product with an activity as a “glycosyl transferase”, preferably it is the molecule of section (a) or (b) of this paragraph.

In particular, it was observed that in plants, especially in Arabidopsis thaliana, increasing or generating the activity of a gene product non-targeted with the activity of a “glycosyl transferase”, preferably being encoded by a gene comprising the nucleic acid sequence SEQ ID NO. 68777, preferably the coding region thereof, conferred the production of or the increase in phenylalanine compared with the wild type control.

The nucleic acid sequence of At3g27540 from Arabidopsis thaliana, e.g. as shown in the respective line in column 5 of Table I, application no. 3, has been published in F. And the activity of the gene product thereof is the activity of glycosyl transferase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “glycosyl transferase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At3g27540, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At3g27540, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At3g27540, or a functional equivalent or a homolog thereof as depicted in column 8 of Table II, application no. 3, preferably a homolog or functional equivalent as depicted in column 8 of Table II B, application no. 3, and being depicted in the same respective line as said At3g27540, and preferably the activity is increased non-targeted.

Accordingly, in one embodiment, the molecule which activity is to be increased in the process of the invention is the gene product with an activity as a “glycosyl transferase”, preferably it is the molecule of section (a) or (b) of this paragraph.

In particular, it was observed that in plants, especially in Arabidopsis thaliana, increasing or generating the activity of a gene product non-targeted with the activity of a “glycosyl transferase”, preferably being encoded by a gene comprising the nucleic acid sequence SEQ ID NO. 68777, preferably the coding region thereof, conferred the production of or the increase in tyrosine compared with the wild type control.

The nucleic acid sequence of At3g61830 from Arabidopsis thaliana, e.g. as shown in the respective line in column 5 of Table I, application no. 3, has been published in F. And the activity of the gene product thereof is the activity of auxin response factor.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “auxin response factor”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At3g61830, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At3g61830, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At3g61830, or a functional equivalent or a homolog thereof as depicted in column 8 of Table II, application no. 3, preferably a homolog or functional equivalent as depicted in column 8 of Table II B, application no. 3, and being depicted in the same respective line as said At3g61830, and preferably the activity is increased non-targeted.

Accordingly, in one embodiment, the molecule which activity is to be increased in the process of the invention is the gene product with an activity as a “auxin response factor”, preferably it is the molecule of section (a) or (b) of this paragraph.

In particular, it was observed that in plants, especially in Arabidopsis thaliana, increasing or generating the activity of a gene product non-targeted with the activity of a “auxin response factor”, preferably being encoded by a gene comprising the nucleic acid sequence SEQ ID NO. 2367, preferably the coding region thereof, conferred the production of or the increase in phenylalanine compared with the wild type control.

The nucleic acid sequence of At3g61830 from Arabidopsis thaliana, e.g. as shown in the respective line in column 5 of Table I, application no. 3, has been published in F. And the activity of the gene product thereof is the activity of auxin response factor.

Accordingly, in one embodiment, the process of the present invention for producing tyrosine in a non-human organism, like a microorganism or a plant or a part thereof, comprises increasing or generating the activity of a gene product with the activity of a gene product conferring the activity of “auxin response factor”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At3g61830, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At3g61830, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At3g61830, or a functional equivalent or a homolog thereof as depicted in column 8 of Table II, application no. 3, preferably a homolog or functional equivalent as depicted in column 8 of Table II B, application no. 3, and being depicted in the same respective line as said At3g61830, and preferably the activity is increased non-targeted.

Accordingly, in one embodiment, the molecule which activity is to be increased in the process of the invention is the gene product with an activity as a “auxin response factor”, preferably it is the molecule of section (a) or (b) of this paragraph.

In particular, it was observed that in plants, especially in Arabidopsis thaliana, increasing or generating the activity of a gene product non-targeted with the activity of a “auxin response factor”, preferably being encoded by a gene comprising the nucleic acid sequence SEQ ID NO. 2367, preferably the coding region thereof, conferred the production of or the increase in tyrosine compared with the wild type control.

The nucleic acid sequence of At3g62930 from Arabidopsis thaliana, e.g. as shown in the respective line in column 5 of Table I, application no. 3, has been published in F. And the activity of the gene product thereof is the activity of monthiol glutaredoxin.

Accordingly, in one embodiment, the process of the present invention for producing aspartate in a non-human organism, like a microorganism or a plant or a part thereof, comprises increasing or generating the activity of a gene product with the activity of a gene product conferring the activity of “monthiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At3g62930, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At3g62930, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At3g62930, or a functional equivalent or a homolog thereof as depicted in column 8 of Table II, application no. 3, preferably a homolog or functional equivalent as depicted in column 8 of Table II B, application no. 3, and being depicted in the same respective line as said At3g62930, and preferably the activity is increased non-targeted.

Accordingly, in one embodiment, the molecule which activity is to be increased in the process of the invention is the gene product with an activity as a “monthiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monthiol glutaredoxin”, preferably being encoded by a gene comprising the nucleic acid sequence SEQ ID NO. 2573, preferably the coding region thereof, conferred the production of or the increase in aspartate compared with the wild type control.

The nucleic acid sequence of At3g62930 from Arabidopsis thaliana, e.g. as shown in the respective line in column 5 of Table I, application no. 3, has been published in F. And the activity of the gene product thereof is the activity of monthiol glutaredoxin.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “monthiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At3g62930, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At3g62930, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At3g62930, or a functional equivalent or a homolog thereof as depicted in column 8 of Table II, application no. 3, preferably a homolog or functional equivalent as depicted in column 8 of Table II B, application no. 3, and being depicted in the same respective line as said At3g62930, and preferably the activity is increased non-targeted.

Accordingly, in one embodiment, the molecule which activity is to be increased in the process of the invention is the gene product with an activity as a “monthiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monthiol glutaredoxin”, preferably being encoded by a gene comprising the nucleic acid sequence SEQ ID NO. 2573, preferably the coding region thereof, conferred the production of or the increase in phenylalanine compared with the wild type control.

The nucleic acid sequence of At3g62950 from Arabidopsis thaliana, e.g. as shown in the respective line in column 5 of Table I, application no. 3, has been published in F. And the activity of the gene product thereof is the activity of glutaredoxin.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At3g62950, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At3g62950, or a functional equivalent or a homolog thereof as depicted in column 8 of Table II, application no. 3, preferably a homolog or functional equivalent as depicted in column 8 of Table II B, application no. 3, and being depicted in the same respective line as said At3g62950, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in phenylalanine compared with the wild type control.

The nucleic acid sequence of At3g62950 from Arabidopsis thaliana, e.g. as shown in the respective line in column 5 of Table I, application no. 3, has been published in F. And the activity of the gene product thereof is the activity of glutaredoxin.

Accordingly, in one embodiment, the process of the present invention for producing tyrosine in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At3g62950, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At3g62950, or a functional equivalent or a homolog thereof as depicted in column 8 of Table II, application no. 3, preferably a homolog or functional equivalent as depicted in column 8 of Table II B, application no. 3, and being depicted in the same respective line as said At3g62950, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in tyrosine compared with the wild type control.

The nucleic acid sequence of At3g62950 from Arabidopsis thaliana, e.g. as shown in the respective line in column 5 of Table I, application no. 3, has been published in F. And the activity of the gene product thereof is the activity of glutaredoxin.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At3g62950, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At3g62950, or a functional equivalent or a homolog thereof as depicted in column 8 of Table II, application no. 3, preferably a homolog or functional equivalent as depicted in column 8 of Table II B, application no. 3, and being depicted in the same respective line as said At3g62950, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in 5-oxoproline compared with the wild type control.

The nucleic acid sequence of At4g15660 from Arabidopsis thaliana, e.g. as shown in the respective line in column 5 of Table I, application no. 3, has been published in F. And the activity of the gene product thereof is the activity of monothiol glutaredoxin.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At4g15660, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At4g15660, or a functional equivalent or a homolog thereof as depicted in column 8 of Table II, application no. 3, preferably a homolog or functional equivalent as depicted in column 8 of Table II B, application no. 3, and being depicted in the same respective line as said At4g15660, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in phenylalanine compared with the wild type control.

The nucleic acid sequence of At4g15660 from Arabidopsis thaliana, e.g. as shown in the respective line in column 5 of Table I, application no. 3, has been published in F. And the activity of the gene product thereof is the activity of monothiol glutaredoxin.

Accordingly, in one embodiment, the process of the present invention for producing tyrosine in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At4g15660, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At4g15660, or a functional equivalent or a homolog thereof as depicted in column 8 of Table II, application no. 3, preferably a homolog or functional equivalent as depicted in column 8 of Table II B, application no. 3, and being depicted in the same respective line as said At4g15660, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in tyrosine compared with the wild type control.

The nucleic acid sequence of At4g15670 from Arabidopsis thaliana, e.g. as shown in the respective line in column 5 of Table I, application no. 3, has been published in F. And the activity of the gene product thereof is the activity of monothiol glutaredoxin.

Accordingly, in one embodiment, the process of the present invention for producing tyrosine in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At4g15670, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At4g15670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At4g15670, or a functional equivalent or a homolog thereof as depicted in column 8 of Table II, application no. 3, preferably a homolog or functional equivalent as depicted in column 8 of Table II B, application no. 3, and being depicted in the same respective line as said At4g15670, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3279, preferably the coding region thereof, conferred the production of or the increase in tyrosine compared with the wild type control.

The nucleic acid sequence of At4g15690 from Arabidopsis thaliana, e.g. as shown in the respective line in column 5 of Table I, application no. 3, has been published in F. And the activity of the gene product thereof is the activity of monothiol glutaredoxin.

Accordingly, in one embodiment, the process of the present invention for producing aspartate in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At4g15690, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At4g15690, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At4g15690, or a functional equivalent or a homolog thereof as depicted in column 8 of Table II, application no. 3, preferably a homolog or functional equivalent as depicted in column 8 of Table II B, application no. 3, and being depicted in the same respective line as said At4g15690, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23844, preferably the coding region thereof, conferred the production of or the increase in aspartate compared with the wild type control.

The nucleic acid sequence of At4g15690 from Arabidopsis thaliana, e.g. as shown in the respective line in column 5 of Table I, application no. 3, has been published in F. And the activity of the gene product thereof is the activity of monothiol glutaredoxin.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At4g15690, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At4g15690, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At4g15690, or a functional equivalent or a homolog thereof as depicted in column 8 of Table II, application no. 3, preferably a homolog or functional equivalent as depicted in column 8 of Table II B, application no. 3, and being depicted in the same respective line as said At4g15690, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23844, preferably the coding region thereof, conferred the production of or the increase in phenylalanine compared with the wild type control.

The nucleic acid sequence of At4g15690 from Arabidopsis thaliana, e.g. as shown in the respective line in column 5 of Table I, application no. 3, has been published in F. And the activity of the gene product thereof is the activity of monothiol glutaredoxin.

Accordingly, in one embodiment, the process of the present invention for producing tyrosine in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At4g15690, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At4g15690, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At4g15690, or a functional equivalent or a homolog thereof as depicted in column 8 of Table II, application no. 3, preferably a homolog or functional equivalent as depicted in column 8 of Table II B, application no. 3, and being depicted in the same respective line as said At4g15690, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23844, preferably the coding region thereof, conferred the production of or the increase in tyrosine compared with the wild type control.

The nucleic acid sequence of At4g15690 from Arabidopsis thaliana, e.g. as shown in the respective line in column 5 of Table I, application no. 3, has been published in F. And the activity of the gene product thereof is the activity of monothiol glutaredoxin.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At4g15690, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At4g15690, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At4g15690, or a functional equivalent or a homolog thereof as depicted in column 8 of Table II, application no. 3, preferably a homolog or functional equivalent as depicted in column 8 of Table II B, application no. 3, and being depicted in the same respective line as said At4g15690, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23844, preferably the coding region thereof, conferred the production of or the increase in 5-oxoproline compared with the wild type control.

The nucleic acid sequence of At4g15700 from Arabidopsis thaliana, e.g. as shown in the respective line in column 5 of Table I, application no. 3, has been published in F. And the activity of the gene product thereof is the activity of monothiol glutaredoxin.

Accordingly, in one embodiment, the process of the present invention for producing tyrosine in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At4g15700, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At4g15700, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At4g15700, or a functional equivalent or a homolog thereof as depicted in column 8 of Table II, application no. 3, preferably a homolog or functional equivalent as depicted in column 8 of Table II B, application no. 3, and being depicted in the same respective line as said At4g15700, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3654, preferably the coding region thereof, conferred the production of or the increase in tyrosine compared with the wild type control.

The nucleic acid sequence of At4g18880 from Arabidopsis thaliana, e.g. as shown in the respective line in column 5 of Table I, application no. 3, has been published in F. And the activity of the gene product thereof is the activity of heat shock transcription factor.

Accordingly, in one embodiment, the process of the present invention for producing serine in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At4g18880, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At4g18880, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At4g18880, or a functional equivalent or a homolog thereof as depicted in column 8 of Table II, application no. 3, preferably a homolog or functional equivalent as depicted in column 8 of Table II B, application no. 3, and being depicted in the same respective line as said At4g18880, and preferably the activity is increased non-targeted.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 24232, preferably the coding region thereof, conferred the production of or the increase in serine compared with the wild type control.

The nucleic acid sequence of At4g26080 from Arabidopsis thaliana, e.g. as shown in the respective line in column 5 of Table I, application no. 3, has been published in F. And the activity of the gene product thereof is the activity of protein phosphatase.

Accordingly, in one embodiment, the process of the present invention for producing alanine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At4g26080, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At4g26080, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At4g26080, or a functional equivalent or a homolog thereof as depicted in column 8 of Table II, application no. 3, preferably a homolog or functional equivalent as depicted in column 8 of Table II B, application no. 3, and being depicted in the same respective line as said At4g26080, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 68849, preferably the coding region thereof, conferred the production of or the increase in alanine compared with the wild type control.

The nucleic acid sequence of At4g26080 from Arabidopsis thaliana, e.g. as shown in the respective line in column 5 of Table I, application no. 3, has been published in F. And the activity of the gene product thereof is the activity of protein phosphatase.

Accordingly, in one embodiment, the process of the present invention for producing aspartate in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At4g26080, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At4g26080, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At4g26080, or a functional equivalent or a homolog thereof as depicted in column 8 of Table II, application no. 3, preferably a homolog or functional equivalent as depicted in column 8 of Table II B, application no. 3, and being depicted in the same respective line as said At4g26080, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 68849, preferably the coding region thereof, conferred the production of or the increase in aspartate compared with the wild type control.

The nucleic acid sequence of At4g26080 from Arabidopsis thaliana, e.g. as shown in the respective line in column 5 of Table I, application no. 3, has been published in F. And the activity of the gene product thereof is the activity of protein phosphatase.

Accordingly, in one embodiment, the process of the present invention for producing citrulline in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At4g26080, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At4g26080, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At4g26080, or a functional equivalent or a homolog thereof as depicted in column 8 of Table II, application no. 3, preferably a homolog or functional equivalent as depicted in column 8 of Table II B, application no. 3, and being depicted in the same respective line as said At4g26080, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 68849, preferably the coding region thereof, conferred the production of or the increase in citrulline compared with the wild type control.

The nucleic acid sequence of At4g32480 from Arabidopsis thaliana, e.g. as shown in the respective line in column 5 of Table I, application no. 3, has been published in F. And the activity of the gene product thereof is the activity of At4g32480-protein.

Accordingly, in one embodiment, the process of the present invention for producing alanine in a non-human organism, like a microorganism or a plant or a part thereof, comprises increasing or generating the activity of a gene product with the activity of a gene product conferring the activity of “At4g32480-protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At4g32480, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At4g32480, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At4g32480, or a functional equivalent or a homolog thereof as depicted in column 8 of Table II, application no. 3, preferably a homolog or functional equivalent as depicted in column 8 of Table II B, application no. 3, and being depicted in the same respective line as said At4g32480, and preferably the activity is increased non-targeted.

Accordingly, in one embodiment, the molecule which activity is to be increased in the process of the invention is the gene product with an activity as a “At4g32480-protein”, preferably it is the molecule of section (a) or (b) of this paragraph.

In particular, it was observed that in plants, especially in Arabidopsis thaliana, increasing or generating the activity of a gene product non-targeted with the activity of a “At4g32480-protein”, preferably being encoded by a gene comprising the nucleic acid sequence SEQ ID NO. 4040, preferably the coding region thereof, conferred the production of or the increase in alanine compared with the wild type control.

The nucleic acid sequence of At4g32480 from Arabidopsis thaliana, e.g. as shown in the respective line in column 5 of Table I, application no. 3, has been published in F. And the activity of the gene product thereof is the activity of At4g32480-protein.

Accordingly, in one embodiment, the process of the present invention for producing asparagine in a non-human organism, like a microorganism or a plant or a part thereof, comprises increasing or generating the activity of a gene product with the activity of a gene product conferring the activity of “At4g32480-protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At4g32480, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At4g32480, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At4g32480, or a functional equivalent or a homolog thereof as depicted in column 8 of Table II, application no. 3, preferably a homolog or functional equivalent as depicted in column 8 of Table II B, application no. 3, and being depicted in the same respective line as said At4g32480, and preferably the activity is increased non-targeted.

Accordingly, in one embodiment, the molecule which activity is to be increased in the process of the invention is the gene product with an activity as a “At4g32480-protein”, preferably it is the molecule of section (a) or (b) of this paragraph.

In particular, it was observed that in plants, especially in Arabidopsis thaliana, increasing or generating the activity of a gene product non-targeted with the activity of a “At4g32480-protein”, preferably being encoded by a gene comprising the nucleic acid sequence SEQ ID NO. 4040, preferably the coding region thereof, conferred the production of or the increase in asparagine compared with the wild type control.

The nucleic acid sequence of At4g32480 from Arabidopsis thaliana, e.g. as shown in the respective line in column 5 of Table I, application no. 3, has been published in F. And the activity of the gene product thereof is the activity of At4g32480-protein.

Accordingly, in one embodiment, the process of the present invention for producing glycine in a non-human organism, like a microorganism or a plant or a part thereof, comprises increasing or generating the activity of a gene product with the activity of a gene product conferring the activity of “At4g32480-protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At4g32480, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At4g32480, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At4g32480, or a functional equivalent or a homolog thereof as depicted in column 8 of Table II, application no. 3, preferably a homolog or functional equivalent as depicted in column 8 of Table II B, application no. 3, and being depicted in the same respective line as said At4g32480, and preferably the activity is increased non-targeted.

Accordingly, in one embodiment, the molecule which activity is to be increased in the process of the invention is the gene product with an activity as a “At4g32480-protein”, preferably it is the molecule of section (a) or (b) of this paragraph.

In particular, it was observed that in plants, especially in Arabidopsis thaliana, increasing or generating the activity of a gene product non-targeted with the activity of a “At4g32480-protein”, preferably being encoded by a gene comprising the nucleic acid sequence SEQ ID NO. 4040, preferably the coding region thereof, conferred the production of or the increase in glycine compared with the wild type control.

The nucleic acid sequence of At4g32480 from Arabidopsis thaliana, e.g. as shown in the respective line in column 5 of Table I, application no. 3, has been published in F. And the activity of the gene product thereof is the activity of At4g32480-protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “At4g32480-protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At4g32480, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At4g32480, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At4g32480, or a functional equivalent or a homolog thereof as depicted in column 8 of Table II, application no. 3, preferably a homolog or functional equivalent as depicted in column 8 of Table II B, application no. 3, and being depicted in the same respective line as said At4g32480, and preferably the activity is increased non-targeted.

Accordingly, in one embodiment, the molecule which activity is to be increased in the process of the invention is the gene product with an activity as a “At4g32480-protein”, preferably it is the molecule of section (a) or (b) of this paragraph.

In particular, it was observed that in plants, especially in Arabidopsis thaliana, increasing or generating the activity of a gene product non-targeted with the activity of a “At4g32480-protein”, preferably being encoded by a gene comprising the nucleic acid sequence SEQ ID NO. 4040, preferably the coding region thereof, conferred the production of or the increase in phenylalanine compared with the wild type control.

The nucleic acid sequence of At4g32480 from Arabidopsis thaliana, e.g. as shown in the respective line in column 5 of Table I, application no. 3, has been published in F. And the activity of the gene product thereof is the activity of At4g32480-protein.

Accordingly, in one embodiment, the process of the present invention for producing serine in a non-human organism, like a microorganism or a plant or a part thereof, comprises increasing or generating the activity of a gene product with the activity of a gene product conferring the activity of “At4g32480-protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At4g32480, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At4g32480, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At4g32480, or a functional equivalent or a homolog thereof as depicted in column 8 of Table II, application no. 3, preferably a homolog or functional equivalent as depicted in column 8 of Table II B, application no. 3, and being depicted in the same respective line as said At4g32480, and preferably the activity is increased non-targeted.

Accordingly, in one embodiment, the molecule which activity is to be increased in the process of the invention is the gene product with an activity as a “At4g32480-protein”, preferably it is the molecule of section (a) or (b) of this paragraph.

In particular, it was observed that in plants, especially in Arabidopsis thaliana, increasing or generating the activity of a gene product non-targeted with the activity of a “At4g32480-protein”, preferably being encoded by a gene comprising the nucleic acid sequence SEQ ID NO. 4040, preferably the coding region thereof, conferred the production of or the increase in serine compared with the wild type control.

The nucleic acid sequence of At4g32480 from Arabidopsis thaliana, e.g. as shown in the respective line in column 5 of Table I, application no. 3, has been published in F. And the activity of the gene product thereof is the activity of At4g32480-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “At4g32480-protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At4g32480, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At4g32480, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At4g32480, or a functional equivalent or a homolog thereof as depicted in column 8 of Table II, application no. 3, preferably a homolog or functional equivalent as depicted in column 8 of Table II B, application no. 3, and being depicted in the same respective line as said At4g32480, and preferably the activity is increased non-targeted.

Accordingly, in one embodiment, the molecule which activity is to be increased in the process of the invention is the gene product with an activity as a “At4g32480-protein”, preferably it is the molecule of section (a) or (b) of this paragraph.

In particular, it was observed that in plants, especially in Arabidopsis thaliana, increasing or generating the activity of a gene product non-targeted with the activity of a “At4g32480-protein”, preferably being encoded by a gene comprising the nucleic acid sequence SEQ ID NO. 4040, preferably the coding region thereof, conferred the production of or the increase in tyrosine compared with the wild type control.

The nucleic acid sequence of At4g33040 from Arabidopsis thaliana, e.g. as shown in the respective line in column 5 of Table I, application no. 3, has been published in F. And the activity of the gene product thereof is the activity of glutaredoxin.

Accordingly, in one embodiment, the process of the present invention for producing aspartate in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At4g33040, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At4g33040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At4g33040, or a functional equivalent or a homolog thereof as depicted in column 8 of Table II, application no. 3, preferably a homolog or functional equivalent as depicted in column 8 of Table II B, application no. 3, and being depicted in the same respective line as said At4g33040, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 4102, preferably the coding region thereof, conferred the production of or the increase in aspartate compared with the wild type control.

The nucleic acid sequence of At4g33040 from Arabidopsis thaliana, e.g. as shown in the respective line in column 5 of Table I, application no. 3, has been published in F. And the activity of the gene product thereof is the activity of glutaredoxin.

Accordingly, in one embodiment, the process of the present invention for producing citrulline in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At4g33040, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At4g33040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At4g33040, or a functional equivalent or a homolog thereof as depicted in column 8 of Table II, application no. 3, preferably a homolog or functional equivalent as depicted in column 8 of Table II B, application no. 3, and being depicted in the same respective line as said At4g33040, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 4102, preferably the coding region thereof, conferred the production of or the increase in citrulline compared with the wild type control.

The nucleic acid sequence of At4g34160 from Arabidopsis thaliana, e.g. as shown in the respective line in column 5 of Table I, application no. 3, has been published in F. And the activity of the gene product thereof is the activity of cyclin D .

Accordingly, in one embodiment, the process of the present invention for producing phenylalanine in a non-human organism, like a microorganism or a plant or a part thereof, comprises increasing or generating the activity of a gene product with the activity of a gene product conferring the activity of “cyclin D”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At4g34160, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At4g34160, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At4g34160, or a functional equivalent or a homolog thereof as depicted in column 8 of Table II, application no. 3, preferably a homolog or functional equivalent as depicted in column 8 of Table II B, application no. 3, and being depicted in the same respective line as said At4g34160, and preferably the activity is increased non-targeted.

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 24311, preferably the coding region thereof, conferred the production of or the increase in phenylalanine compared with the wild type control.

The nucleic acid sequence of At4g35310 from Arabidopsis thaliana, e.g. as shown in the respective line in column 5 of Table I, application no. 3, has been published in F. And the activity of the gene product thereof is the activity of calcium-dependent protein kinase.

Accordingly, in one embodiment, the process of the present invention for producing asparagine in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At4g35310, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At4g35310, or a functional equivalent or a homolog thereof as depicted in column 8 of Table II, application no. 3, preferably a homolog or functional equivalent as depicted in column 8 of Table II B, application no. 3, and being depicted in the same respective line as said At4g35310, and preferably the activity is increased non-targeted.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in asparagine compared with the wild type control.

The nucleic acid sequence of At4g35310 from Arabidopsis thaliana, e.g. as shown in the respective line in column 5 of Table I, application no. 3, has been published in F. And the activity of the gene product thereof is the activity of calcium-dependent protein kinase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At4g35310, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At4g35310, or a functional equivalent or a homolog thereof as depicted in column 8 of Table II, application no. 3, preferably a homolog or functional equivalent as depicted in column 8 of Table II B, application no. 3, and being depicted in the same respective line as said At4g35310, and preferably the activity is increased non-targeted.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in phenylalanine compared with the wild type control.

The nucleic acid sequence of At4g35310 from Arabidopsis thaliana, e.g. as shown in the respective line in column 5 of Table I, application no. 3, has been published in F. And the activity of the gene product thereof is the activity of calcium-dependent protein kinase.

Accordingly, in one embodiment, the process of the present invention for producing serine in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At4g35310, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At4g35310, or a functional equivalent or a homolog thereof as depicted in column 8 of Table II, application no. 3, preferably a homolog or functional equivalent as depicted in column 8 of Table II B, application no. 3, and being depicted in the same respective line as said At4g35310, and preferably the activity is increased non-targeted.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in serine compared with the wild type control.

The nucleic acid sequence of At4g35310 from Arabidopsis thaliana, e.g. as shown in the respective line in column 5 of Table I, application no. 3, has been published in F. And the activity of the gene product thereof is the activity of calcium-dependent protein kinase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At4g35310, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At4g35310, or a functional equivalent or a homolog thereof as depicted in column 8 of Table II, application no. 3, preferably a homolog or functional equivalent as depicted in column 8 of Table II B, application no. 3, and being depicted in the same respective line as said At4g35310, and preferably the activity is increased non-targeted.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in tyrosine compared with the wild type control.

The nucleic acid sequence of At4g35310 from Arabidopsis thaliana, e.g. as shown in the respective line in column 5 of Table I, application no. 3, has been published in F. And the activity of the gene product thereof is the activity of calcium-dependent protein kinase.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At4g35310, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At4g35310, or a functional equivalent or a homolog thereof as depicted in column 8 of Table II, application no. 3, preferably a homolog or functional equivalent as depicted in column 8 of Table II B, application no. 3, and being depicted in the same respective line as said At4g35310, and preferably the activity is increased non-targeted.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in 5-oxoproline compared with the wild type control.

The nucleic acid sequence of At5g02760 from Arabidopsis thaliana, e.g. as shown in the respective line in column 5 of Table I, application no. 3, has been published in F. And the activity of the gene product thereof is the activity of protein phosphatase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At5g02760, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At5g02760, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At5g02760, or a functional equivalent or a homolog thereof as depicted in column 8 of Table II, application no. 3, preferably a homolog or functional equivalent as depicted in column 8 of Table II B, application no. 3, and being depicted in the same respective line as said At5g02760, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 68987, preferably the coding region thereof, conferred the production of or the increase in tyrosine compared with the wild type control.

The nucleic acid sequence of At5g03720 from Arabidopsis thaliana, e.g. as shown in the respective line in column 5 of Table I, application no. 3, has been published in F. And the activity of the gene product thereof is the activity of heat shock transcription factor.

Accordingly, in one embodiment, the process of the present invention for producing aspartate in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At5g03720, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At5g03720, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At5g03720, or a functional equivalent or a homolog thereof as depicted in column 8 of Table II, application no. 3, preferably a homolog or functional equivalent as depicted in column 8 of Table II B, application no. 3, and being depicted in the same respective line as said At5g03720, and preferably the activity is increased non-targeted.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 24438, preferably the coding region thereof, conferred the production of or the increase in aspartate compared with the wild type control.

The nucleic acid sequence of At5g03720 from Arabidopsis thaliana, e.g. as shown in the respective line in column 5 of Table I, application no. 3, has been published in F. And the activity of the gene product thereof is the activity of heat shock transcription factor.

Accordingly, in one embodiment, the process of the present invention for producing 3,4-dihydroxyphenylalanine (dopa) in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At5g03720, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At5g03720, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At5g03720, or a functional equivalent or a homolog thereof as depicted in column 8 of Table II, application no. 3, preferably a homolog or functional equivalent as depicted in column 8 of Table II B, application no. 3, and being depicted in the same respective line as said At5g03720, and preferably the activity is increased non-targeted.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 24438, preferably the coding region thereof, conferred the production of or the increase in 3,4-dihydroxyphenylalanine (dopa) compared with the wild type control.

The nucleic acid sequence of At5g07200 from Arabidopsis thaliana, e.g. as shown in the respective line in column 5 of Table I, application no. 3, has been published in F. And the activity of the gene product thereof is the activity of gibberellin 20-oxidase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “gibberellin 20-oxidase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At5g07200, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At5g07200, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At5g07200, or a functional equivalent or a homolog thereof as depicted in column 8 of Table II, application no. 3, preferably a homolog or functional equivalent as depicted in column 8 of Table II B, application no. 3, and being depicted in the same respective line as said At5g07200, and preferably the activity is increased non-targeted.

Accordingly, in one embodiment, the molecule which activity is to be increased in the process of the invention is the gene product with an activity as a “gibberellin 20-oxidase”, preferably it is the molecule of section (a) or (b) of this paragraph.

In particular, it was observed that in plants, especially in Arabidopsis thaliana, increasing or generating the activity of a gene product non-targeted with the activity of a “gibberellin 20-oxidase”, preferably being encoded by a gene comprising the nucleic acid sequence SEQ ID NO. 24492, preferably the coding region thereof, conferred the production of or the increase in phenylalanine compared with the wild type control.

The nucleic acid sequence of At5g07200 from Arabidopsis thaliana, e.g. as shown in the respective line in column 5 of Table I, application no. 3, has been published in F. And the activity of the gene product thereof is the activity of gibberellin 20-oxidase.

Accordingly, in one embodiment, the process of the present invention for producing serine in a non-human organism, like a microorganism or a plant or a part thereof, comprises increasing or generating the activity of a gene product with the activity of a gene product conferring the activity of “gibberellin 20-oxidase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At5g07200, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At5g07200, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At5g07200, or a functional equivalent or a homolog thereof as depicted in column 8 of Table II, application no. 3, preferably a homolog or functional equivalent as depicted in column 8 of Table II B, application no. 3, and being depicted in the same respective line as said At5g07200, and preferably the activity is increased non-targeted.

Accordingly, in one embodiment, the molecule which activity is to be increased in the process of the invention is the gene product with an activity as a “gibberellin 20-oxidase”, preferably it is the molecule of section (a) or (b) of this paragraph.

In particular, it was observed that in plants, especially in Arabidopsis thaliana, increasing or generating the activity of a gene product non-targeted with the activity of a “gibberellin 20-oxidase”, preferably being encoded by a gene comprising the nucleic acid sequence SEQ ID NO. 24492, preferably the coding region thereof, conferred the production of or the increase in serine compared with the wild type control.

The nucleic acid sequence of At5g10820 from Arabidopsis thaliana, e.g. as shown in the respective line in column 5 of Table I, application no. 3, has been published in F. And the activity of the gene product thereof is the activity of integral membrane transporter family protein.

Accordingly, in one embodiment, the process of the present invention for producing serine in a non-human organism, like a microorganism or a plant or a part thereof, comprises increasing or generating the activity of a gene product with the activity of a gene product conferring the activity of “integral membrane transporter family protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At5g10820, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At5g10820, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At5g10820, or a functional equivalent or a homolog thereof as depicted in column 8 of Table II, application no. 3, preferably a homolog or functional equivalent as depicted in column 8 of Table II B, application no. 3, and being depicted in the same respective line as said At5g10820, and preferably the activity is increased non-targeted.

Accordingly, in one embodiment, the molecule which activity is to be increased in the process of the invention is the gene product with an activity as a “integral membrane transporter family protein”, preferably it is the molecule of section (a) or (b) of this paragraph.

In particular, it was observed that in plants, especially in Arabidopsis thaliana, increasing or generating the activity of a gene product non-targeted with the activity of a “integral membrane transporter family protein”, preferably being encoded by a gene comprising the nucleic acid sequence SEQ ID NO. 25222, preferably the coding region thereof, conferred the production of or the increase in serine compared with the wild type control.

The nucleic acid sequence of At5g14070 from Arabidopsis thaliana, e.g. as shown in the respective line in column 5 of Table I, application no. 3, has been published in F. And the activity of the gene product thereof is the activity of glutaredoxin.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At5g14070, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At5g14070, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At5g14070, or a functional equivalent or a homolog thereof as depicted in column 8 of Table II, application no. 3, preferably a homolog or functional equivalent as depicted in column 8 of Table II B, application no. 3, and being depicted in the same respective line as said At5g14070, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 69275, preferably the coding region thereof, conferred the production of or the increase in phenylalanine compared with the wild type control.

The nucleic acid sequence of At5g16650 from Arabidopsis thaliana, e.g. as shown in the respective line in column 5 of Table I, application no. 3, has been published in F. And the activity of the gene product thereof is the activity of At5g16650-protein.

Accordingly, in one embodiment, the process of the present invention for producing asparagine in a non-human organism, like a microorganism or a plant or a part thereof, comprises increasing or generating the activity of a gene product with the activity of a gene product conferring the activity of “At5g16650-protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At5g16650, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At5g16650, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At5g16650, or a functional equivalent or a homolog thereof as depicted in column 8 of Table II, application no. 3, preferably a homolog or functional equivalent as depicted in column 8 of Table II B, application no. 3, and being depicted in the same respective line as said At5g16650, and preferably the activity is increased non-targeted.

Accordingly, in one embodiment, the molecule which activity is to be increased in the process of the invention is the gene product with an activity as a “At5g16650-protein”, preferably it is the molecule of section (a) or (b) of this paragraph.

In particular, it was observed that in plants, especially in Arabidopsis thaliana, increasing or generating the activity of a gene product non-targeted with the activity of a “At5g16650-protein”, preferably being encoded by a gene comprising the nucleic acid sequence SEQ ID NO. 25283, preferably the coding region thereof, conferred the production of or the increase in asparagine compared with the wild type control.

The nucleic acid sequence of At5g16650 from Arabidopsis thaliana, e.g. as shown in the respective line in column 5 of Table I, application no. 3, has been published in F. And the activity of the gene product thereof is the activity of At5g16650-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “At5g16650-protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At5g16650, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At5g16650, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At5g16650, or a functional equivalent or a homolog thereof as depicted in column 8 of Table II, application no. 3, preferably a homolog or functional equivalent as depicted in column 8 of Table II B, application no. 3, and being depicted in the same respective line as said At5g16650, and preferably the activity is increased non-targeted.

Accordingly, in one embodiment, the molecule which activity is to be increased in the process of the invention is the gene product with an activity as a “At5g16650-protein”, preferably it is the molecule of section (a) or (b) of this paragraph.

In particular, it was observed that in plants, especially in Arabidopsis thaliana, increasing or generating the activity of a gene product non-targeted with the activity of a “At5g16650-protein”, preferably being encoded by a gene comprising the nucleic acid sequence SEQ ID NO. 25283, preferably the coding region thereof, conferred the production of or the increase in aspartate compared with the wild type control.

The nucleic acid sequence of At5g16650 from Arabidopsis thaliana, e.g. as shown in the respective line in column 5 of Table I, application no. 3, has been published in F. And the activity of the gene product thereof is the activity of At5g16650-protein.

Accordingly, in one embodiment, the process of the present invention for producing serine in a non-human organism, like a microorganism or a plant or a part thereof, comprises increasing or generating the activity of a gene product with the activity of a gene product conferring the activity of “At5g16650-protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At5g16650, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At5g16650, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At5g16650, or a functional equivalent or a homolog thereof as depicted in column 8 of Table II, application no. 3, preferably a homolog or functional equivalent as depicted in column 8 of Table II B, application no. 3, and being depicted in the same respective line as said At5g16650, and preferably the activity is increased non-targeted.

Accordingly, in one embodiment, the molecule which activity is to be increased in the process of the invention is the gene product with an activity as a “At5g16650-protein”, preferably it is the molecule of section (a) or (b) of this paragraph.

In particular, it was observed that in plants, especially in Arabidopsis thaliana, increasing or generating the activity of a gene product non-targeted with the activity of a “At5g16650-protein”, preferably being encoded by a gene comprising the nucleic acid sequence SEQ ID NO. 25283, preferably the coding region thereof, conferred the production of or the increase in serine compared with the wild type control.

The nucleic acid sequence of At5g16650 from Arabidopsis thaliana, e.g. as shown in the respective line in column 5 of Table I, application no. 3, has been published in F. And the activity of the gene product thereof is the activity of At5g16650-protein.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “At5g16650-protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At5g16650, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At5g16650, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At5g16650, or a functional equivalent or a homolog thereof as depicted in column 8 of Table II, application no. 3, preferably a homolog or functional equivalent as depicted in column 8 of Table II B, application no. 3, and being depicted in the same respective line as said At5g16650, and preferably the activity is increased non-targeted.

Accordingly, in one embodiment, the molecule which activity is to be increased in the process of the invention is the gene product with an activity as a “At5g16650-protein”, preferably it is the molecule of section (a) or (b) of this paragraph.

In particular, it was observed that in plants, especially in Arabidopsis thaliana, increasing or generating the activity of a gene product non-targeted with the activity of a “At5g16650-protein”, preferably being encoded by a gene comprising the nucleic acid sequence SEQ ID NO. 25283, preferably the coding region thereof, conferred the production of or the increase in 5-oxoproline compared with the wild type control.

The nucleic acid sequence of At5g18600 from Arabidopsis thaliana, e.g. as shown in the respective line in column 5 of Table I, application no. 3, has been published in F. And the activity of the gene product thereof is the activity of monothiol glutaredoxin.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At5g18600, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At5g18600, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At5g18600, or a functional equivalent or a homolog thereof as depicted in column 8 of Table II, application no. 3, preferably a homolog or functional equivalent as depicted in column 8 of Table II B, application no. 3, and being depicted in the same respective line as said At5g18600, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 4904, preferably the coding region thereof, conferred the production of or the increase in phenylalanine compared with the wild type control.

The nucleic acid sequence of At5g18600 from Arabidopsis thaliana, e.g. as shown in the respective line in column 5 of Table I, application no. 3, has been published in F. And the activity of the gene product thereof is the activity of monothiol glutaredoxin.

Accordingly, in one embodiment, the process of the present invention for producing tyrosine in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At5g18600, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At5g18600, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At5g18600, or a functional equivalent or a homolog thereof as depicted in column 8 of Table II, application no. 3, preferably a homolog or functional equivalent as depicted in column 8 of Table II B, application no. 3, and being depicted in the same respective line as said At5g18600, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 4904, preferably the coding region thereof, conferred the production of or the increase in tyrosine compared with the wild type control.

The nucleic acid sequence of At5g21910 from Arabidopsis thaliana, e.g. as shown in the respective line in column 5 of Table I, application no. 3, has been published in F. And the activity of the gene product thereof is the activity of at5g21910-protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “at5g21910-protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At5g21910, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At5g21910, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At5g21910, or a functional equivalent or a homolog thereof as depicted in column 8 of Table II, application no. 3, preferably a homolog or functional equivalent as depicted in column 8 of Table II B, application no. 3, and being depicted in the same respective line as said At5g21910, and preferably the activity is increased non-targeted.

Accordingly, in one embodiment, the molecule which activity is to be increased in the process of the invention is the gene product with an activity as a “at5g21910-protein”, preferably it is the molecule of section (a) or (b) of this paragraph.

In particular, it was observed that in plants, especially in Arabidopsis thaliana, increasing or generating the activity of a gene product non-targeted with the activity of a “at5g21910-protein”, preferably being encoded by a gene comprising the nucleic acid sequence SEQ ID NO. 69553, preferably the coding region thereof, conferred the production of or the increase in phenylalanine compared with the wild type control.

The nucleic acid sequence of At5g39760 from Arabidopsis thaliana, e.g. as shown in the respective line in column 5 of Table I, application no. 3, has been published in F. And the activity of the gene product thereof is the activity of transcription factor.

Accordingly, in one embodiment, the process of the present invention for producing tyrosine in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At5g39760, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At5g39760, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At5g39760, or a functional equivalent or a homolog thereof as depicted in column 8 of Table II, application no. 3, preferably a homolog or functional equivalent as depicted in column 8 of Table II B, application no. 3, and being depicted in the same respective line as said At5g39760, and preferably the activity is increased non-targeted.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 25428, preferably the coding region thereof, conferred the production of or the increase in tyrosine compared with the wild type control.

The nucleic acid sequence of At5g39950 from Arabidopsis thaliana, e.g. as shown in the respective line in column 5 of Table I, application no. 3, has been published in F. And the activity of the gene product thereof is the activity of thioredoxin.

Accordingly, in one embodiment, the process of the present invention for producing serine in a non-human organism, like a microorganism or a plant or a part thereof, comprises increasing or generating the activity of a gene product with the activity of a gene product conferring the activity of “thioredoxin”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At5g39950, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At5g39950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At5g39950, or a functional equivalent or a homolog thereof as depicted in column 8 of Table II, application no. 3, preferably a homolog or functional equivalent as depicted in column 8 of Table II B, application no. 3, and being depicted in the same respective line as said At5g39950, and preferably the activity is increased non-targeted.

Accordingly, in one embodiment, the molecule which activity is to be increased in the process of the invention is the gene product with an activity as a “thioredoxin”, preferably it is the molecule of section (a) or (b) of this paragraph.

In particular, it was observed that in plants, especially in Arabidopsis thaliana, increasing or generating the activity of a gene product non-targeted with the activity of a “thioredoxin”, preferably being encoded by a gene comprising the nucleic acid sequence SEQ ID NO. 69574, preferably the coding region thereof, conferred the production of or the increase in serine compared with the wild type control.

The nucleic acid sequence of At5g57050 from Arabidopsis thaliana, e.g. as shown in the respective line in column 5 of Table I, application no. 3, has been published in F. And the activity of the gene product thereof is the activity of protein phosphatase.

Accordingly, in one embodiment, the process of the present invention for producing alanine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At5g57050, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At5g57050, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At5g57050, or a functional equivalent or a homolog thereof as depicted in column 8 of Table II, application no. 3, preferably a homolog or functional equivalent as depicted in column 8 of Table II B, application no. 3, and being depicted in the same respective line as said At5g57050, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 5318, preferably the coding region thereof, conferred the production of or the increase in alanine compared with the wild type control.

The nucleic acid sequence of At5g57050 from Arabidopsis thaliana, e.g. as shown in the respective line in column 5 of Table I, application no. 3, has been published in F. And the activity of the gene product thereof is the activity of protein phosphatase.

Accordingly, in one embodiment, the process of the present invention for producing citrulline in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At5g57050, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At5g57050, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At5g57050, or a functional equivalent or a homolog thereof as depicted in column 8 of Table II, application no. 3, preferably a homolog or functional equivalent as depicted in column 8 of Table II B, application no. 3, and being depicted in the same respective line as said At5g57050, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 5318, preferably the coding region thereof, conferred the production of or the increase in citrulline compared with the wild type control.

The nucleic acid sequence of At5g59220 from Arabidopsis thaliana, e.g. as shown in the respective line in column 5 of Table I, application no. 3, has been published in F. And the activity of the gene product thereof is the activity of protein phosphatase.

Accordingly, in one embodiment, the process of the present invention for producing citrulline in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At5g59220, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At5g59220, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At5g59220, or a functional equivalent or a homolog thereof as depicted in column 8 of Table II, application no. 3, preferably a homolog or functional equivalent as depicted in column 8 of Table II B, application no. 3, and being depicted in the same respective line as said At5g59220, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 25498, preferably the coding region thereof, conferred the production of or the increase in citrulline compared with the wild type control.

The nucleic acid sequence of At5g60110 from Arabidopsis thaliana, e.g. as shown in the respective line in column 5 of Table I, application no. 3, has been published in F. And the activity of the gene product thereof is the activity of RNA-binding protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “RNA-binding protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At5g60110, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At5g60110, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At5g60110, or a functional equivalent or a homolog thereof as depicted in column 8 of Table II, application no. 3, preferably a homolog or functional equivalent as depicted in column 8 of Table II B, application no. 3, and being depicted in the same respective line as said At5g60110, and preferably the activity is increased non-targeted.

Accordingly, in one embodiment, the molecule which activity is to be increased in the process of the invention is the gene product with an activity as a “RNA-binding protein”, preferably it is the molecule of section (a) or (b) of this paragraph.

In particular, it was observed that in plants, especially in Arabidopsis thaliana, increasing or generating the activity of a gene product non-targeted with the activity of a “RNA-binding protein”, preferably being encoded by a gene comprising the nucleic acid sequence SEQ ID NO. 70006, preferably the coding region thereof, conferred the production of or the increase in phenylalanine compared with the wild type control.

The nucleic acid sequence of At5g60110 from Arabidopsis thaliana, e.g. as shown in the respective line in column 5 of Table I, application no. 3, has been published in F. And the activity of the gene product thereof is the activity of RNA-binding protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “RNA-binding protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At5g60110, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At5g60110, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At5g60110, or a functional equivalent or a homolog thereof as depicted in column 8 of Table II, application no. 3, preferably a homolog or functional equivalent as depicted in column 8 of Table II B, application no. 3, and being depicted in the same respective line as said At5g60110, and preferably the activity is increased non-targeted.

Accordingly, in one embodiment, the molecule which activity is to be increased in the process of the invention is the gene product with an activity as a “RNA-binding protein”, preferably it is the molecule of section (a) or (b) of this paragraph.

In particular, it was observed that in plants, especially in Arabidopsis thaliana, increasing or generating the activity of a gene product non-targeted with the activity of a “RNA-binding protein”, preferably being encoded by a gene comprising the nucleic acid sequence SEQ ID NO. 70006, preferably the coding region thereof, conferred the production of or the increase in tyrosine compared with the wild type control.

The nucleic acid sequence of At5g63680 from Arabidopsis thaliana, e.g. as shown in the respective line in column 5 of Table I, application no. 3, has been published in F. And the activity of the gene product thereof is the activity of pyruvate kinase.

Accordingly, in one embodiment, the process of the present invention for producing alanine in a non-human organism, like a microorganism or a plant or a part thereof, comprises increasing or generating the activity of a gene product with the activity of a gene product conferring the activity of “pyruvate kinase”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At5g63680, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At5g63680, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At5g63680, or a functional equivalent or a homolog thereof as depicted in column 8 of Table II, application no. 3, preferably a homolog or functional equivalent as depicted in column 8 of Table II B, application no. 3, and being depicted in the same respective line as said At5g63680, and preferably the activity is increased plastidic.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 70038, preferably the coding region thereof, conferred the production of or the increase in alanine compared with the wild type control.

The nucleic acid sequence of At5g64920 from Arabidopsis thaliana, e.g. as shown in the respective line in column 5 of Table I, application no. 3, has been published in F. And the activity of the gene product thereof is the activity of zinc finger protein.

Accordingly, in one embodiment, the process of the present invention for producing alanine in a non-human organism, like a microorganism or a plant or a part thereof, comprises increasing or generating the activity of a gene product with the activity of a gene product conferring the activity of “zinc finger protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At5g64920, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At5g64920, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At5g64920, or a functional equivalent or a homolog thereof as depicted in column 8 of Table II, application no. 3, preferably a homolog or functional equivalent as depicted in column 8 of Table II B, application no. 3, and being depicted in the same respective line as said At5g64920, and preferably the activity is increased non-targeted.

Accordingly, in one embodiment, the molecule which activity is to be increased in the process of the invention is the gene product with an activity as a “zinc finger protein”, preferably it is the molecule of section (a) or (b) of this paragraph.

In particular, it was observed that in plants, especially in Arabidopsis thaliana, increasing or generating the activity of a gene product non-targeted with the activity of a “zinc finger protein”, preferably being encoded by a gene comprising the nucleic acid sequence SEQ ID NO. 5493, preferably the coding region thereof, conferred the production of or the increase in alanine compared with the wild type control.

The nucleic acid sequence of At5g64920 from Arabidopsis thaliana, e.g. as shown in the respective line in column 5 of Table I, application no. 3, has been published in F. And the activity of the gene product thereof is the activity of zinc finger protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “zinc finger protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At5g64920, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At5g64920, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At5g64920, or a functional equivalent or a homolog thereof as depicted in column 8 of Table II, application no. 3, preferably a homolog or functional equivalent as depicted in column 8 of Table II B, application no. 3, and being depicted in the same respective line as said At5g64920, and preferably the activity is increased non-targeted.

Accordingly, in one embodiment, the molecule which activity is to be increased in the process of the invention is the gene product with an activity as a “zinc finger protein”, preferably it is the molecule of section (a) or (b) of this paragraph.

In particular, it was observed that in plants, especially in Arabidopsis thaliana, increasing or generating the activity of a gene product non-targeted with the activity of a “zinc finger protein”, preferably being encoded by a gene comprising the nucleic acid sequence SEQ ID NO. 5493, preferably the coding region thereof, conferred the production of or the increase in phenylalanine compared with the wild type control.

The nucleic acid sequence of At5g64920 from Arabidopsis thaliana, e.g. as shown in the respective line in column 5 of Table I, application no. 3, has been published in F. And the activity of the gene product thereof is the activity of zinc finger protein.

Accordingly, in one embodiment, the process of the present invention for producing serine in a non-human organism, like a microorganism or a plant or a part thereof, comprises increasing or generating the activity of a gene product with the activity of a gene product conferring the activity of “zinc finger protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At5g64920, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At5g64920, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At5g64920, or a functional equivalent or a homolog thereof as depicted in column 8 of Table II, application no. 3, preferably a homolog or functional equivalent as depicted in column 8 of Table II B, application no. 3, and being depicted in the same respective line as said At5g64920, and preferably the activity is increased non-targeted.

Accordingly, in one embodiment, the molecule which activity is to be increased in the process of the invention is the gene product with an activity as a “zinc finger protein”, preferably it is the molecule of section (a) or (b) of this paragraph.

In particular, it was observed that in plants, especially in Arabidopsis thaliana, increasing or generating the activity of a gene product non-targeted with the activity of a “zinc finger protein”, preferably being encoded by a gene comprising the nucleic acid sequence SEQ ID NO. 5493, preferably the coding region thereof, conferred the production of or the increase in serine compared with the wild type control.

The nucleic acid sequence of At5g64920 from Arabidopsis thaliana, e.g. as shown in the respective line in column 5 of Table I, application no. 3, has been published in F. And the activity of the gene product thereof is the activity of zinc finger protein.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “zinc finger protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said At5g64920, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said At5g64920, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said At5g64920, or a functional equivalent or a homolog thereof as depicted in column 8 of Table II, application no. 3, preferably a homolog or functional equivalent as depicted in column 8 of Table II B, application no. 3, and being depicted in the same respective line as said At5g64920, and preferably the activity is increased non-targeted.

Accordingly, in one embodiment, the molecule which activity is to be increased in the process of the invention is the gene product with an activity as a “zinc finger protein”, preferably it is the molecule of section (a) or (b) of this paragraph.

In particular, it was observed that in plants, especially in Arabidopsis thaliana, increasing or generating the activity of a gene product non-targeted with the activity of a “zinc finger protein”, preferably being encoded by a gene comprising the nucleic acid sequence SEQ ID NO. 5493, preferably the coding region thereof, conferred the production of or the increase in 5-oxoproline compared with the wild type control.

The nucleic acid sequence of AvinDRAFT_{—}0035 from Azotobacter vinelandii, e.g. as shown in the respective line in column 5 of Table I, application no. 3, has been published in D. And the activity of the gene product thereof is the activity of CDP-diacylglycerol-serine O-phosphatidyltransferase.

Accordingly, in one embodiment, the process of the present invention for producing aspartate in a non-human organism, like a microorganism or a plant or a part thereof, comprises increasing or generating the activity of a gene product with the activity of a gene product conferring the activity of “CDP-diacylglycerol-serine O-phosphatidyltransferase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}0035, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}0035, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}0035, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}0035, and preferably the activity is increased non-targeted.

Accordingly, in one embodiment, the molecule which activity is to be increased in the process of the invention is the gene product with an activity as a “CDP-diacylglycerol-serine O-phosphatidyltransferase”, preferably it is the molecule of section (a) or (b) of this paragraph.

In particular, it was observed that in plants, especially in Arabidopsis thaliana, increasing or generating the activity of a gene product non-targeted with the activity of a “CDP-diacylglycerolserine O-phosphatidyltransferase”, preferably being encoded by a gene comprising the nucleic acid sequence SEQ ID NO. 70583, preferably the coding region thereof, conferred the production of or the increase in aspartate compared with the wild type control.

The nucleic acid sequence of AvinDRAFT_{—}0035 from Azotobacter vinelandii, e.g. as shown in the respective line in column 5 of Table I, application no. 3, has been published in D. And the activity of the gene product thereof is the activity of CDP-diacylglycerol-serine O-phosphatidyltransferase.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “CDP-diacylglycerol-serine O-phosphatidyltransferase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}0035, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}0035, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}0035, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}0035, and preferably the activity is increased non-targeted.

Accordingly, in one embodiment, the molecule which activity is to be increased in the process of the invention is the gene product with an activity as a “CDP-diacylglycerol-serine O-phosphatidyltransferase”, preferably it is the molecule of section (a) or (b) of this paragraph.

In particular, it was observed that in plants, especially in Arabidopsis thaliana, increasing or generating the activity of a gene product non-targeted with the activity of a “CDP-diacylglycerolserine O-phosphatidyltransferase”, preferably being encoded by a gene comprising the nucleic acid sequence SEQ ID NO. 70583, preferably the coding region thereof, conferred the production of or the increase in 5-oxoproline compared with the wild type control.

The nucleic acid sequence of AVINDRAFT_{—}0539 from AZOTOBACTER VINELANDII, e.g. as shown in the respective line in column 5 of Table I, application no. 3, has been published in D. And the activity of the gene product thereof is the activity of Glycine cleavage system T aminomethyltransferase.

Accordingly, in one embodiment, the process of the present invention for producing serine in a non-human organism, like a microorganism or a plant or a part thereof, comprises increasing or generating the activity of a gene product with the activity of a gene product conferring the activity of “Glycine cleavage system T aminomethyltransferase”, especially from AZOTOBACTER VINELANDII or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AVINDRAFT_{—}0539, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AVINDRAFT_{—}0539, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AVINDRAFT_{—}0539, or a functional equivalent or a homolog thereof as depicted in column 8 of Table II, application no. 3, preferably a homolog or functional equivalent as depicted in column 8 of Table II B, application no. 3, and being depicted in the same respective line as said AVINDRAFT_{—}0539, and preferably the activity is increased non-targeted.

Accordingly, in one embodiment, the molecule which activity is to be increased in the process of the invention is the gene product with an activity as a “Glycine cleavage system T aminomethyltransferase”, preferably it is the molecule of section (a) or (b) of this paragraph.

In particular, it was observed that in plants, especially in Arabidopsis thaliana, increasing or generating the activity of a gene product non-targeted with the activity of a “Glycine cleavage system T aminomethyltransferase”, preferably being encoded by a gene comprising the nucleic acid sequence SEQ ID NO. 70715, preferably the coding region thereof, conferred the production of or the increase in serine compared with the wild type control.

The nucleic acid sequence of AvinDRAFT_{—}1045 from Azotobacter vinelandii, e.g. as shown in the respective line in column 5 of Table I, application no. 3, has been published in D. And the activity of the gene product thereof is the activity of transcriptional regulator.

Accordingly, in one embodiment, the process of the present invention for producing serine in a non-human organism, like a microorganism or a plant or a part thereof, comprises increasing or generating the activity of a gene product with the activity of a gene product conferring the activity of “transcriptional regulator”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}1045, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}1045, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}1045, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}1045, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 25676, preferably the coding region thereof, conferred the production of or the increase in serine compared with the wild type control.

The nucleic acid sequence of AvinDRAFT_{—}1398 from Azotobacter vinelandii, e.g. as shown in the respective line in column 5 of Table I, application no. 3, has been published in D. And the activity of the gene product thereof is the activity of adenylylsulfate kinase.

Accordingly, in one embodiment, the process of the present invention for producing aspartate in a non-human organism, like a microorganism or a plant or a part thereof, comprises increasing or generating the activity of a gene product with the activity of a gene product conferring the activity of “adenylylsulfate kinase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}1398, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}1398, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}1398, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}1398, and preferably the activity is increased non-targeted.

Accordingly, in one embodiment, the molecule which activity is to be increased in the process of the invention is the gene product with an activity as a “adenylylsulfate kinase”, preferably it is the molecule of section (a) or (b) of this paragraph.

In particular, it was observed that in plants, especially in Arabidopsis thaliana, increasing or generating the activity of a gene product non-targeted with the activity of a “adenylylsulfate kinase”, preferably being encoded by a gene comprising the nucleic acid sequence SEQ ID NO. 25780, preferably the coding region thereof, conferred the production of or the increase in aspartate compared with the wild type control.

The nucleic acid sequence of AvinDRAFT_{—}1495 from Azotobacter vinelandii, e.g. as shown in the respective line in column 5 of Table I, application no. 3, has been published in D. And the activity of the gene product thereof is the activity of malic enzyme.

Accordingly, in one embodiment, the process of the present invention for producing alanine in a non-human organism, like a microorganism or a plant or a part thereof, comprises increasing or generating the activity of a gene product with the activity of a gene product conferring the activity of “malic enzyme”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}1495, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}1495, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}1495, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}1495, and preferably the activity is increased non-targeted.

Accordingly, in one embodiment, the molecule which activity is to be increased in the process of the invention is the gene product with an activity as a “malic enzyme”, preferably it is the molecule of section (a) or (b) of this paragraph.

In particular, it was observed that in plants, especially in Arabidopsis thaliana, increasing or generating the activity of a gene product non-targeted with the activity of a “malic enzyme”, preferably being encoded by a gene comprising the nucleic acid sequence SEQ ID NO. 5557, preferably the coding region thereof, conferred the production of or the increase in alanine compared with the wild type control.

The nucleic acid sequence of AvinDRAFT_{—}1495 from Azotobacter vinelandii, e.g. as shown in the respective line in column 5 of Table I, application no. 3, has been published in D. And the activity of the gene product thereof is the activity of malic enzyme.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “malic enzyme”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}1495, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}1495, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}1495, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}1495, and preferably the activity is increased non-targeted.

Accordingly, in one embodiment, the molecule which activity is to be increased in the process of the invention is the gene product with an activity as a “malic enzyme”, preferably it is the molecule of section (a) or (b) of this paragraph.

In particular, it was observed that in plants, especially in Arabidopsis thaliana, increasing or generating the activity of a gene product non-targeted with the activity of a “malic enzyme”, preferably being encoded by a gene comprising the nucleic acid sequence SEQ ID NO. 5557, preferably the coding region thereof, conferred the production of or the increase in phenylalanine compared with the wild type control.

The nucleic acid sequence of AvinDRAFT_{—}1495 from Azotobacter vinelandii, e.g. as shown in the respective line in column 5 of Table I, application no. 3, has been published in D. And the activity of the gene product thereof is the activity of malic enzyme.

Accordingly, in one embodiment, the process of the present invention for producing serine in a non-human organism, like a microorganism or a plant or a part thereof, comprises increasing or generating the activity of a gene product with the activity of a gene product conferring the activity of “malic enzyme”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}1495, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}1495, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}1495, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}1495, and preferably the activity is increased non-targeted.

Accordingly, in one embodiment, the molecule which activity is to be increased in the process of the invention is the gene product with an activity as a “malic enzyme”, preferably it is the molecule of section (a) or (b) of this paragraph.

In particular, it was observed that in plants, especially in Arabidopsis thaliana, increasing or generating the activity of a gene product non-targeted with the activity of a “malic enzyme”, preferably being encoded by a gene comprising the nucleic acid sequence SEQ ID NO. 5557, preferably the coding region thereof, conferred the production of or the increase in serine compared with the wild type control.

The nucleic acid sequence of AvinDRAFT_{—}1624 from Azotobacter vinelandii, e.g. as shown in the respective line in column 5 of Table I, application no. 3, has been published in D. And the activity of the gene product thereof is the activity of sec-independent protein translocase.

Accordingly, in one embodiment, the process of the present invention for producing alanine in a non-human organism, like a microorganism or a plant or a part thereof, comprises increasing or generating the activity of a gene product with the activity of a gene product conferring the activity of “sec-independent protein translocase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}1624, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}1624, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}1624, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}1624, and preferably the activity is increased non-targeted.

Accordingly, in one embodiment, the molecule which activity is to be increased in the process of the invention is the gene product with an activity as a “sec-independent protein translocase”, preferably it is the molecule of section (a) or (b) of this paragraph.

In particular, it was observed that in plants, especially in Arabidopsis thaliana, increasing or generating the activity of a gene product non-targeted with the activity of a “sec-independent protein translocase”, preferably being encoded by a gene comprising the nucleic acid sequence SEQ ID NO. 26196, preferably the coding region thereof, conferred the production of or the increase in alanine compared with the wild type control.

The nucleic acid sequence of AvinDRAFT_{—}1806 from Azotobacter vinelandii, e.g. as shown in the respective line in column 5 of Table I, application no. 3, has been published in D. And the activity of the gene product thereof is the activity of pyruvate kinase.

Accordingly, in one embodiment, the process of the present invention for producing alanine in a non-human organism, like a microorganism or a plant or a part thereof, comprises increasing or generating the activity of a gene product with the activity of a gene product conferring the activity of “pyruvate kinase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}1806, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}1806, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}1806, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}1806, and preferably the activity is increased non-targeted.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product non-targeted with the activity of a “pyruvate kinase”, preferably being encoded by a gene comprising the nucleic acid sequence SEQ ID NO. 26434, preferably the coding region thereof, conferred the production of or the increase in alanine compared with the wild type control.

The nucleic acid sequence of AvinDRAFT_{—}2091 from Azotobacter vinelandii, e.g. as shown in the respective line in column 5 of Table I, application no. 3, has been published in D. And the activity of the gene product thereof is the activity of beta-hydroxylase.

Accordingly, in one embodiment, the process of the present invention for producing alanine in a non-human organism, like a microorganism or a plant or a part thereof, comprises increasing or generating the activity of a gene product with the activity of a gene product conferring the activity of “beta-hydroxylase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}2091, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}2091, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}2091, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}2091, and preferably the activity is increased non-targeted.

Accordingly, in one embodiment, the molecule which activity is to be increased in the process of the invention is the gene product with an activity as a “beta-hydroxylase”, preferably it is the molecule of section (a) or (b) of this paragraph.

In particular, it was observed that in plants, especially in Arabidopsis thaliana, increasing or generating the activity of a gene product non-targeted with the activity of a “beta-hydroxylase”, preferably being encoded by a gene comprising the nucleic acid sequence SEQ ID NO. 6040, preferably the coding region thereof, conferred the production of or the increase in alanine compared with the wild type control.

The nucleic acid sequence of AvinDRAFT_{—}2091 from Azotobacter vinelandii, e.g. as shown in the respective line in column 5 of Table I, application no. 3, has been published in D. And the activity of the gene product thereof is the activity of beta-hydroxylase.

Accordingly, in one embodiment, the process of the present invention for producing aspartate in a non-human organism, like a microorganism or a plant or a part thereof, comprises increasing or generating the activity of a gene product with the activity of a gene product conferring the activity of “beta-hydroxylase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}2091, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}2091, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}2091, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}2091, and preferably the activity is increased non-targeted.

Accordingly, in one embodiment, the molecule which activity is to be increased in the process of the invention is the gene product with an activity as a “beta-hydroxylase”, preferably it is the molecule of section (a) or (b) of this paragraph.

In particular, it was observed that in plants, especially in Arabidopsis thaliana, increasing or generating the activity of a gene product non-targeted with the activity of a “beta-hydroxylase”, preferably being encoded by a gene comprising the nucleic acid sequence SEQ ID NO. 6040, preferably the coding region thereof, conferred the production of or the increase in aspartate compared with the wild type control.

The nucleic acid sequence of AvinDRAFT_{—}2091 from Azotobacter vinelandii, e.g. as shown in the respective line in column 5 of Table I, application no. 3, has been published in D. And the activity of the gene product thereof is the activity of beta-hydroxylase.

Accordingly, in one embodiment, the process of the present invention for producing serine in a non-human organism, like a microorganism or a plant or a part thereof, comprises increasing or generating the activity of a gene product with the activity of a gene product conferring the activity of “beta-hydroxylase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}2091, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}2091, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}2091, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}2091, and preferably the activity is increased non-targeted.

Accordingly, in one embodiment, the molecule which activity is to be increased in the process of the invention is the gene product with an activity as a “beta-hydroxylase”, preferably it is the molecule of section (a) or (b) of this paragraph.

In particular, it was observed that in plants, especially in Arabidopsis thaliana, increasing or generating the activity of a gene product non-targeted with the activity of a “beta-hydroxylase”, preferably being encoded by a gene comprising the nucleic acid sequence SEQ ID NO. 6040, preferably the coding region thereof, conferred the production of or the increase in serine compared with the wild type control.

The nucleic acid sequence of AvinDRAFT_{—}2344 from Azotobacter vinelandii, e.g. as shown in the respective line in column 5 of Table I, application no. 3, has been published in D. And the activity of the gene product thereof is the activity of elongation factor Tu.

Accordingly, in one embodiment, the process of the present invention for producing aspartate in a non-human organism, like a microorganism or a plant or a part thereof, comprises increasing or generating the activity of a gene product with the activity of a gene product conferring the activity of “elongation factor Tu”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}2344, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}2344, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}2344, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}2344, and preferably the activity is increased non-targeted.

Accordingly, in one embodiment, the molecule which activity is to be increased in the process of the invention is the gene product with an activity as a “elongation factor Tu”, preferably it is the molecule of section (a) or (b) of this paragraph.

In particular, it was observed that in plants, especially in Arabidopsis thaliana, increasing or generating the activity of a gene product non-targeted with the activity of a “elongation factor Tu”, preferably being encoded by a gene comprising the nucleic acid sequence SEQ ID NO. 27021, preferably the coding region thereof, conferred the production of or the increase in aspartate compared with the wild type control.

The nucleic acid sequence of AvinDRAFT_{—}2344 from Azotobacter vinelandii, e.g. as shown in the respective line in column 5 of Table I, application no. 3, has been published in D. And the activity of the gene product thereof is the activity of elongation factor Tu.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “elongation factor Tu”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}2344, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}2344, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}2344, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}2344, and preferably the activity is increased non-targeted.

Accordingly, in one embodiment, the molecule which activity is to be increased in the process of the invention is the gene product with an activity as a “elongation factor Tu”, preferably it is the molecule of section (a) or (b) of this paragraph.

In particular, it was observed that in plants, especially in Arabidopsis thaliana, increasing or generating the activity of a gene product non-targeted with the activity of a “elongation factor Tu”, preferably being encoded by a gene comprising the nucleic acid sequence SEQ ID NO. 27021, preferably the coding region thereof, conferred the production of or the increase in phenylalanine compared with the wild type control.

The nucleic acid sequence of AvinDRAFT_{—}2344 from Azotobacter vinelandii, e.g. as shown in the respective line in column 5 of Table I, application no. 3, has been published in D. And the activity of the gene product thereof is the activity of elongation factor Tu.

Accordingly, in one embodiment, the process of the present invention for producing serine in a non-human organism, like a microorganism or a plant or a part thereof, comprises increasing or generating the activity of a gene product with the activity of a gene product conferring the activity of “elongation factor Tu”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}2344, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}2344, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}2344, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}2344, and preferably the activity is increased non-targeted.

Accordingly, in one embodiment, the molecule which activity is to be increased in the process of the invention is the gene product with an activity as a “elongation factor Tu”, preferably it is the molecule of section (a) or (b) of this paragraph.

In particular, it was observed that in plants, especially in Arabidopsis thaliana, increasing or generating the activity of a gene product non-targeted with the activity of a “elongation factor Tu”, preferably being encoded by a gene comprising the nucleic acid sequence SEQ ID NO. 27021, preferably the coding region thereof, conferred the production of or the increase in serine compared with the wild type control.

The nucleic acid sequence of AvinDRAFT_{—}2344 from Azotobacter vinelandii, e.g. as shown in the respective line in column 5 of Table I, application no. 3, has been published in D. And the activity of the gene product thereof is the activity of elongation factor Tu.

Accordingly, in one embodiment, the process of the present invention for producing tyrosine in a non-human organism, like a microorganism or a plant or a part thereof, comprises increasing or generating the activity of a gene product with the activity of a gene product conferring the activity of “elongation factor Tu”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}2344, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}2344, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}2344, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}2344, and preferably the activity is increased non-targeted.

Accordingly, in one embodiment, the molecule which activity is to be increased in the process of the invention is the gene product with an activity as a “elongation factor Tu”, preferably it is the molecule of section (a) or (b) of this paragraph.

In particular, it was observed that in plants, especially in Arabidopsis thaliana, increasing or generating the activity of a gene product non-targeted with the activity of a “elongation factor Tu”, preferably being encoded by a gene comprising the nucleic acid sequence SEQ ID NO. 27021, preferably the coding region thereof, conferred the production of or the increase in tyrosine compared with the wild type control.

The nucleic acid sequence of AvinDRAFT_{—}2344 from Azotobacter vinelandii, e.g. as shown in the respective line in column 5 of Table I, application no. 3, has been published in D. And the activity of the gene product thereof is the activity of elongation factor Tu.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “elongation factor Tu”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}2344, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}2344, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}2344, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}2344, and preferably the activity is increased non-targeted.

Accordingly, in one embodiment, the molecule which activity is to be increased in the process of the invention is the gene product with an activity as a “elongation factor Tu”, preferably it is the molecule of section (a) or (b) of this paragraph.

In particular, it was observed that in plants, especially in Arabidopsis thaliana, increasing or generating the activity of a gene product non-targeted with the activity of a “elongation factor Tu”, preferably being encoded by a gene comprising the nucleic acid sequence SEQ ID NO. 27021, preferably the coding region thereof, conferred the production of or the increase in 5-oxoproline compared with the wild type control.

The nucleic acid sequence of AvinDRAFT_{—}2365 from Azotobacter vinelandii, e.g. as shown in the respective line in column 5 of Table I, application no. 3, has been published in D. And the activity of the gene product thereof is the activity of 30S ribosomal protein S3.

Accordingly, in one embodiment, the process of the present invention for producing tyrosine in a non-human organism, like a microorganism or a plant or a part thereof, comprises increasing or generating the activity of a gene product with the activity of a gene product conferring the activity of “30S ribosomal protein S3”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}2365, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}2365, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}2365, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}2365, and preferably the activity is increased non-targeted.

Accordingly, in one embodiment, the molecule which activity is to be increased in the process of the invention is the gene product with an activity as a “305 ribosomal protein S3”, preferably it is the molecule of section (a) or (b) of this paragraph.

In particular, it was observed that in plants, especially in Arabidopsis thaliana, increasing or generating the activity of a gene product non-targeted with the activity of a “305 ribosomal protein S3”, preferably being encoded by a gene comprising the nucleic acid sequence SEQ ID NO. 71325, preferably the coding region thereof, conferred the production of or the increase in tyrosine compared with the wild type control.

The nucleic acid sequence of AvinDRAFT_{—}2369 from Azotobacter vinelandii, e.g. as shown in the respective line in column 5 of Table I, application no. 3, has been published in D. And the activity of the gene product thereof is the activity of 50S ribosomal protein L14.

Accordingly, in one embodiment, the process of the present invention for producing asparagine in a non-human organism, like a microorganism or a plant or a part thereof, comprises increasing or generating the activity of a gene product with the activity of a gene product conferring the activity of “505 ribosomal protein L14”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}2369, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}2369, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}2369, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}2369, and preferably the activity is increased non-targeted.

Accordingly, in one embodiment, the molecule which activity is to be increased in the process of the invention is the gene product with an activity as a “505 ribosomal protein L14”, preferably it is the molecule of section (a) or (b) of this paragraph.

In particular, it was observed that in plants, especially in Arabidopsis thaliana, increasing or generating the activity of a gene product non-targeted with the activity of a “505 ribosomal protein L14”, preferably being encoded by a gene comprising the nucleic acid sequence SEQ ID NO. 71763, preferably the coding region thereof, conferred the production of or the increase in asparagine compared with the wild type control.

The nucleic acid sequence of AvinDRAFT_{—}2521 from Azotobacter vinelandii, e.g. as shown in the respective line in column 5 of Table I, application no. 3, has been published in D. And the activity of the gene product thereof is the activity of ABC transporter permease protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “ABC transporter permease protein”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}2521, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}2521, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}2521, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}2521, and preferably the activity is increased non-targeted.

Accordingly, in one embodiment, the molecule which activity is to be increased in the process of the invention is the gene product with an activity as a “ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “ABC transporter permease protein”, preferably being encoded by a gene comprising the nucleic acid sequence SEQ ID NO. 27882, preferably the coding region thereof, conferred the production of or the increase in tyrosine compared with the wild type control.

The nucleic acid sequence of AvinDRAFT_{—}2754 from Azotobacter vinelandii, e.g. as shown in the respective line in column 5 of Table I, application no. 3, has been published in D. And the activity of the gene product thereof is the activity of acyl-CoA synthase.

Accordingly, in one embodiment, the process of the present invention for producing aspartate in a non-human organism, like a microorganism or a plant or a part thereof, comprises increasing or generating the activity of a gene product with the activity of a gene product conferring the activity of “acyl-CoA synthase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}2754, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}2754, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}2754, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}2754, and preferably the activity is increased non-targeted.

Accordingly, in one embodiment, the molecule which activity is to be increased in the process of the invention is the gene product with an activity as a “acyl-CoA synthase”, preferably it is the molecule of section (a) or (b) of this paragraph.

In particular, it was observed that in plants, especially in Arabidopsis thaliana, increasing or generating the activity of a gene product non-targeted with the activity of a “acyl-CoA synthase”, preferably being encoded by a gene comprising the nucleic acid sequence SEQ ID NO. 28040, preferably the coding region thereof, conferred the production of or the increase in aspartate compared with the wild type control.

The nucleic acid sequence of AvinDRAFT_{—}2754 from Azotobacter vinelandii, e.g. as shown in the respective line in column 5 of Table I, application no. 3, has been published in D. And the activity of the gene product thereof is the activity of acyl-CoA synthase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “acyl-CoA synthase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}2754, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}2754, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}2754, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}2754, and preferably the activity is increased non-targeted.

Accordingly, in one embodiment, the molecule which activity is to be increased in the process of the invention is the gene product with an activity as a “acyl-CoA synthase”, preferably it is the molecule of section (a) or (b) of this paragraph.

In particular, it was observed that in plants, especially in Arabidopsis thaliana, increasing or generating the activity of a gene product non-targeted with the activity of a “acyl-CoA synthase”, preferably being encoded by a gene comprising the nucleic acid sequence SEQ ID NO. 28040, preferably the coding region thereof, conferred the production of or the increase in tyrosine compared with the wild type control.

The nucleic acid sequence of AvinDRAFT_{—}2754 from Azotobacter vinelandii, e.g. as shown in the respective line in column 5 of Table I, application no. 3, has been published in D. And the activity of the gene product thereof is the activity of acyl-CoA synthase.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “acyl-CoA synthase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}2754, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}2754, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}2754, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}2754, and preferably the activity is increased non-targeted.

Accordingly, in one embodiment, the molecule which activity is to be increased in the process of the invention is the gene product with an activity as a “acyl-CoA synthase”, preferably it is the molecule of section (a) or (b) of this paragraph.

In particular, it was observed that in plants, especially in Arabidopsis thaliana, increasing or generating the activity of a gene product non-targeted with the activity of a “acyl-CoA synthase”, preferably being encoded by a gene comprising the nucleic acid sequence SEQ ID NO. 28040, preferably the coding region thereof, conferred the production of or the increase in 5-oxoproline compared with the wild type control.

The nucleic acid sequence of AvinDRAFT_{—}3028 from Azotobacter vinelandii, e.g. as shown in the respective line in column 5 of Table I, application no. 3, has been published in D. And the activity of the gene product thereof is the activity of 2-oxoglutarate dehydrogenase E1 subunit.

Accordingly, in one embodiment, the process of the present invention for producing aspartate in a non-human organism, like a microorganism or a plant or a part thereof, comprises increasing or generating the activity of a gene product with the activity of a gene product conferring the activity of “2-oxoglutarate dehydrogenase E1 subunit”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}3028, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}3028, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}3028, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}3028, and preferably the activity is increased non-targeted.

Accordingly, in one embodiment, the molecule which activity is to be increased in the process of the invention is the gene product with an activity as a “2-oxoglutarate dehydrogenase E1 subunit”, preferably it is the molecule of section (a) or (b) of this paragraph.

In particular, it was observed that in plants, especially in Arabidopsis thaliana, increasing or generating the activity of a gene product non-targeted with the activity of a “2-oxoglutarate dehydrogenase E1 subunit”, preferably being encoded by a gene comprising the nucleic acid sequence SEQ ID NO. 6075, preferably the coding region thereof, conferred the production of or the increase in aspartate compared with the wild type control.

The nucleic acid sequence of AvinDRAFT_{—}3028 from Azotobacter vinelandii, e.g. as shown in the respective line in column 5 of Table I, application no. 3, has been published in D. And the activity of the gene product thereof is the activity of 2-oxoglutarate dehydrogenase E1 subunit.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “2-oxoglutarate dehydrogenase E1 subunit”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}3028, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}3028, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}3028, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}3028, and preferably the activity is increased non-targeted.

Accordingly, in one embodiment, the molecule which activity is to be increased in the process of the invention is the gene product with an activity as a “2-oxoglutarate dehydrogenase E1 subunit”, preferably it is the molecule of section (a) or (b) of this paragraph.

In particular, it was observed that in plants, especially in Arabidopsis thaliana, increasing or generating the activity of a gene product non-targeted with the activity of a “2-oxoglutarate dehydrogenase E1 subunit”, preferably being encoded by a gene comprising the nucleic acid sequence SEQ ID NO. 6075, preferably the coding region thereof, conferred the production of or the increase in 5-oxoproline compared with the wild type control.

The nucleic acid sequence of AvinDRAFT_{—}3135 from Azotobacter vinelandii, e.g. as shown in the respective line in column 5 of Table I, application no. 3, has been published in D. And the activity of the gene product thereof is the activity of GDP-mannose dehydrogenase.

Accordingly, in one embodiment, the process of the present invention for producing serine in a non-human organism, like a microorganism or a plant or a part thereof, comprises increasing or generating the activity of a gene product with the activity of a gene product conferring the activity of “GDP-mannose dehydrogenase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}3135, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}3135, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}3135, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}3135, and preferably the activity is increased non-targeted.

Accordingly, in one embodiment, the molecule which activity is to be increased in the process of the invention is the gene product with an activity as a “GDP-mannose dehydrogenase”, preferably it is the molecule of section (a) or (b) of this paragraph.

In particular, it was observed that in plants, especially in Arabidopsis thaliana, increasing or generating the activity of a gene product non-targeted with the activity of a “GDP-mannose dehydrogenase”, preferably being encoded by a gene comprising the nucleic acid sequence SEQ ID NO. 72346, preferably the coding region thereof, conferred the production of or the increase in serine compared with the wild type control.

The nucleic acid sequence of AvinDRAFT_{—}3186 from Azotobacter vinelandii, e.g. as shown in the respective line in column 5 of Table I, application no. 3, has been published in D. And the activity of the gene product thereof is the activity of transcriptional regulator.

Accordingly, in one embodiment, the process of the present invention for producing serine in a non-human organism, like a microorganism or a plant or a part thereof, comprises increasing or generating the activity of a gene product with the activity of a gene product conferring the activity of “transcriptional regulator”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}3186, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}3186, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}3186, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}3186, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 29246, preferably the coding region thereof, conferred the production of or the increase in serine compared with the wild type control.

The nucleic acid sequence of AvinDRAFT_{—}3209 from Azotobacter vinelandii, e.g. as shown in the respective line in column 5 of Table I, application no. 3, has been published in D. And the activity of the gene product thereof is the activity of transcriptional regulator.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “transcriptional regulator”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}3209, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}3209, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}3209, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}3209, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 29286, preferably the coding region thereof, conferred the production of or the increase in phenylalanine compared with the wild type control.

The nucleic acid sequence of AvinDRAFT_{—}3253 from Azotobacter vinelandii, e.g. as shown in the respective line in column 5 of Table I, application no. 3, has been published in D. And the activity of the gene product thereof is the activity of oxidoreductase.

Accordingly, in one embodiment, the process of the present invention for producing aspartate in a non-human organism, like a microorganism or a plant or a part thereof, comprises increasing or generating the activity of a gene product with the activity of a gene product conferring the activity of “oxidoreductase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}3253, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}3253, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}3253, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}3253, and preferably the activity is increased non-targeted.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 29500, preferably the coding region thereof, conferred the production of or the increase in aspartate compared with the wild type control.

The nucleic acid sequence of AvinDRAFT_{—}3253 from Azotobacter vinelandii, e.g. as shown in the respective line in column 5 of Table I, application no. 3, has been published in D. And the activity of the gene product thereof is the activity of oxidoreductase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “oxidoreductase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}3253, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}3253, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}3253, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}3253, and preferably the activity is increased non-targeted.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 29500, preferably the coding region thereof, conferred the production of or the increase in tyrosine compared with the wild type control.

The nucleic acid sequence of AvinDRAFT_{—}3556 from Azotobacter vinelandii, e.g. as shown in the respective line in column 5 of Table I, application no. 3, has been published in D. And the activity of the gene product thereof is the activity of lysyl-tRNA synthetase.

Accordingly, in one embodiment, the process of the present invention for producing citrulline in a non-human organism, like a microorganism or a plant or a part thereof, comprises increasing or generating the activity of a gene product with the activity of a gene product conferring the activity of “lysyl-tRNA synthetase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}3556, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}3556, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}3556, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}3556, and preferably the activity is increased non-targeted.

Accordingly, in one embodiment, the molecule which activity is to be increased in the process of the invention is the gene product with an activity as a “lysyl-tRNA synthetase”, preferably it is the molecule of section (a) or (b) of this paragraph.

In particular, it was observed that in plants, especially in Arabidopsis thaliana, increasing or generating the activity of a gene product non-targeted with the activity of a “lysyl-tRNA synthetase”, preferably being encoded by a gene comprising the nucleic acid sequence SEQ ID NO. 30039, preferably the coding region thereof, conferred the production of or the increase in citrulline compared with the wild type control.

The nucleic acid sequence of AVINDRAFT_{—}4128 from AZOTOBACTER VINELANDII, e.g. as shown in the respective line in column 5 of Table I, application no. 3, has been published in D. And the activity of the gene product thereof is the activity of ABC transporter component.

Accordingly, in one embodiment, the process of the present invention for producing asparagine in a non-human organism, like a microorganism or a plant or a part thereof, comprises increasing or generating the activity of a gene product with the activity of a gene product conferring the activity of “ABC transporter component”, especially from AZOTOBACTER VINELANDII or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AVINDRAFT_{—}4128, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AVINDRAFT_{—}4128, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AVINDRAFT_{—}4128, or a functional equivalent or a homolog thereof as depicted in column 8 of Table II, application no. 3, preferably a homolog or functional equivalent as depicted in column 8 of Table II B, application no. 3, and being depicted in the same respective line as said AVINDRAFT_{—}4128, and preferably the activity is increased non-targeted.

Accordingly, in one embodiment, the molecule which activity is to be increased in the process of the invention is the gene product with an activity as a “ABC transporter component”, preferably it is the molecule of section (a) or (b) of this paragraph.

In particular, it was observed that in plants, especially in Arabidopsis thaliana, increasing or generating the activity of a gene product non-targeted with the activity of a “ABC transporter component”, preferably being encoded by a gene comprising the nucleic acid sequence SEQ ID NO. 72520, preferably the coding region thereof, conferred the production of or the increase in asparagine compared with the wild type control.

The nucleic acid sequence of AvinDRAFT_{—}4384 from Azotobacter vinelandii, e.g. as shown in the respective line in column 5 of Table I, application no. 3, has been published in D. And the activity of the gene product thereof is the activity of glucose-1-phosphate cytidylyltransferase.

Accordingly, in one embodiment, the process of the present invention for producing aspartate in a non-human organism, like a microorganism or a plant or a part thereof, comprises increasing or generating the activity of a gene product with the activity of a gene product conferring the activity of “glucose-1-phosphate cytidylyltransferase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}4384, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}4384, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}4384, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}4384, and preferably the activity is increased non-targeted.

Accordingly, in one embodiment, the molecule which activity is to be increased in the process of the invention is the gene product with an activity as a “glucose-1-phosphate cytidylyltransferase”, preferably it is the molecule of section (a) or (b) of this paragraph.

In particular, it was observed that in plants, especially in Arabidopsis thaliana, increasing or generating the activity of a gene product non-targeted with the activity of a “glucose-1-phosphate cytidylyltransferase”, preferably being encoded by a gene comprising the nucleic acid sequence SEQ ID NO. 31717, preferably the coding region thereof, conferred the production of or the increase in aspartate compared with the wild type control.

The nucleic acid sequence of AvinDRAFT_{—}4385 from Azotobacter vinelandii, e.g. as shown in the respective line in column 5 of Table I, application no. 3, has been published in D. And the activity of the gene product thereof is the activity of CDP-glucose-4,6-dehydratase.

Accordingly, in one embodiment, the process of the present invention for producing glycine in a non-human organism, like a microorganism or a plant or a part thereof, comprises increasing or generating the activity of a gene product with the activity of a gene product conferring the activity of “CDP-glucose-4,6-dehydratase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}4385, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}4385, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}4385, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}4385, and preferably the activity is increased non-targeted.

Accordingly, in one embodiment, the molecule which activity is to be increased in the process of the invention is the gene product with an activity as a “CDP-glucose-4,6-dehydratase”, preferably it is the molecule of section (a) or (b) of this paragraph.

In particular, it was observed that in plants, especially in Arabidopsis thaliana, increasing or generating the activity of a gene product non-targeted with the activity of a “CDP-glucose-4,6-dehydratase”, preferably being encoded by a gene comprising the nucleic acid sequence SEQ ID NO. 72935, preferably the coding region thereof, conferred the production of or the increase in glycine compared with the wild type control.

The nucleic acid sequence of AvinDRAFT_{—}4420 from Azotobacter vinelandii, e.g. as shown in the respective line in column 5 of Table I, application no. 3, has been published in D. And the activity of the gene product thereof is the activity of enoyl-CoA hydratase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “enoyl-CoA hydratase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}4420, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}4420, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}4420, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}4420, and preferably the activity is increased non-targeted.

Accordingly, in one embodiment, the molecule which activity is to be increased in the process of the invention is the gene product with an activity as a “enoyl-CoA hydratase”, preferably it is the molecule of section (a) or (b) of this paragraph.

In particular, it was observed that in plants, especially in Arabidopsis thaliana, increasing or generating the activity of a gene product non-targeted with the activity of a “enoyl-CoA hydratase”, preferably being encoded by a gene comprising the nucleic acid sequence SEQ ID NO. 73038, preferably the coding region thereof, conferred the production of or the increase in phenylalanine compared with the wild type control.

The nucleic acid sequence of AvinDRAFT_{—}4446 from Azotobacter vinelandii, e.g. as shown in the respective line in column 5 of Table I, application no. 3, has been published in D. And the activity of the gene product thereof is the activity of glutathione peroxidase.

Accordingly, in one embodiment, the process of the present invention for producing alanine in a non-human organism, like a microorganism or a plant or a part thereof, comprises increasing or generating the activity of a gene product with the activity of a gene product conferring the activity of “glutathione peroxidase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}4446, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}4446, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}4446, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}4446, and preferably the activity is increased non-targeted.

Accordingly, in one embodiment, the molecule which activity is to be increased in the process of the invention is the gene product with an activity as a “glutathione peroxidase”, preferably it is the molecule of section (a) or (b) of this paragraph.

In particular, it was observed that in plants, especially in Arabidopsis thaliana, increasing or generating the activity of a gene product non-targeted with the activity of a “glutathione peroxidase”, preferably being encoded by a gene comprising the nucleic acid sequence SEQ ID NO. 73284, preferably the coding region thereof, conferred the production of or the increase in alanine compared with the wild type control.

The nucleic acid sequence of AvinDRAFT_{—}4562 from Azotobacter vinelandii, e.g. as shown in the respective line in column 5 of Table I, application no. 3, has been published in D. And the activity of the gene product thereof is the activity of aminotransferase.

Accordingly, in one embodiment, the process of the present invention for producing asparagine in a non-human organism, like a microorganism or a plant or a part thereof, comprises increasing or generating the activity of a gene product with the activity of a gene product conferring the activity of “aminotransferase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}4562, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}4562, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}4562, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}4562, and preferably the activity is increased non-targeted.

Accordingly, in one embodiment, the molecule which activity is to be increased in the process of the invention is the gene product with an activity as a “aminotransferase”, preferably it is the molecule of section (a) or (b) of this paragraph.

In particular, it was observed that in plants, especially in Arabidopsis thaliana, increasing or generating the activity of a gene product non-targeted with the activity of a “aminotransferase”, preferably being encoded by a gene comprising the nucleic acid sequence SEQ ID NO. 31926, preferably the coding region thereof, conferred the production of or the increase in asparagine compared with the wild type control.

The nucleic acid sequence of AvinDRAFT_{—}4562 from Azotobacter vinelandii, e.g. as shown in the respective line in column 5 of Table I, application no. 3, has been published in D. And the activity of the gene product thereof is the activity of aminotransferase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “aminotransferase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}4562, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}4562, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}4562, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}4562, and preferably the activity is increased non-targeted.

Accordingly, in one embodiment, the molecule which activity is to be increased in the process of the invention is the gene product with an activity as a “aminotransferase”, preferably it is the molecule of section (a) or (b) of this paragraph.

In particular, it was observed that in plants, especially in Arabidopsis thaliana, increasing or generating the activity of a gene product non-targeted with the activity of a “aminotransferase”, preferably being encoded by a gene comprising the nucleic acid sequence SEQ ID NO. 31926, preferably the coding region thereof, conferred the production of or the increase in phenylalanine compared with the wild type control.

The nucleic acid sequence of AvinDRAFT_{—}4606 from Azotobacter vinelandii, e.g. as shown in the respective line in column 5 of Table I, application no. 3, has been published in D. And the activity of the gene product thereof is the activity of NADH-quinone oxidoreductase subunit.

Accordingly, in one embodiment, the process of the present invention for producing tyrosine in a non-human organism, like a microorganism or a plant or a part thereof, comprises increasing or generating the activity of a gene product with the activity of a gene product conferring the activity of “NADH-quinone oxidoreductase subunit”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}4606, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}4606, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}4606, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}4606, and preferably the activity is increased non-targeted.

Accordingly, in one embodiment, the molecule which activity is to be increased in the process of the invention is the gene product with an activity as a “NADH-quinone oxidoreductase subunit”, preferably it is the molecule of section (a) or (b) of this paragraph.

In particular, it was observed that in plants, especially in Arabidopsis thaliana, increasing or generating the activity of a gene product non-targeted with the activity of a “NADH-quinone oxidoreductase subunit”, preferably being encoded by a gene comprising the nucleic acid sequence SEQ ID NO. 73719, preferably the coding region thereof, conferred the production of or the increase in tyrosine compared with the wild type control.

The nucleic acid sequence of AvinDRAFT_{—}4836 from Azotobacter vinelandii, e.g. as shown in the respective line in column 5 of Table I, application no. 3, has been published in D. And the activity of the gene product thereof is the activity of glutamate-ammonia-ligase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “glutamate-ammonia-ligase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}4836, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}4836, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}4836, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}4836, and preferably the activity is increased non-targeted.

Accordingly, in one embodiment, the molecule which activity is to be increased in the process of the invention is the gene product with an activity as a “glutamate-ammonia-ligase”, preferably it is the molecule of section (a) or (b) of this paragraph.

In particular, it was observed that in plants, especially in Arabidopsis thaliana, increasing or generating the activity of a gene product non-targeted with the activity of a “glutamate-ammonia-ligase”, preferably being encoded by a gene comprising the nucleic acid sequence SEQ ID NO. 74163, preferably the coding region thereof, conferred the production of or the increase in phenylalanine compared with the wild type control.

The nucleic acid sequence of AvinDRAFT_{—}4836 from Azotobacter vinelandii, e.g. as shown in the respective line in column 5 of Table I, application no. 3, has been published in D. And the activity of the gene product thereof is the activity of glutamate-ammonia-ligase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “glutamate-ammonia-ligase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}4836, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}4836, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}4836, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}4836, and preferably the activity is increased non-targeted.

Accordingly, in one embodiment, the molecule which activity is to be increased in the process of the invention is the gene product with an activity as a “glutamate-ammonia-ligase”, preferably it is the molecule of section (a) or (b) of this paragraph.

In particular, it was observed that in plants, especially in Arabidopsis thaliana, increasing or generating the activity of a gene product non-targeted with the activity of a “glutamate-ammonia-ligase”, preferably being encoded by a gene comprising the nucleic acid sequence SEQ ID NO. 74163, preferably the coding region thereof, conferred the production of or the increase in tyrosine compared with the wild type control.

The nucleic acid sequence of AvinDRAFT_{—}4989 from Azotobacter vinelandii, e.g. as shown in the respective line in column 5 of Table I, application no. 3, has been published in D. And the activity of the gene product thereof is the activity of RNA polymerase sigma factor.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “RNA polymerase sigma factor”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}4989, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}4989, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}4989, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}4989, and preferably the activity is increased non-targeted.

Accordingly, in one embodiment, the molecule which activity is to be increased in the process of the invention is the gene product with an activity as a “RNA polymerase sigma factor”, preferably it is the molecule of section (a) or (b) of this paragraph.

In particular, it was observed that in plants, especially in Arabidopsis thaliana, increasing or generating the activity of a gene product non-targeted with the activity of a “RNA polymerase sigma factor”, preferably being encoded by a gene comprising the nucleic acid sequence SEQ ID NO. 74452, preferably the coding region thereof, conferred the production of or the increase in 5-oxoproline compared with the wild type control.

The nucleic acid sequence of AvinDRAFT_{—}5103 from Azotobacter vinelandii, e.g. as shown in the respective line in column 5 of Table I, application no. 3, has been published in D. And the activity of the gene product thereof is the activity of hydrolase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “hydrolase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}5103, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}5103, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}5103, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}5103, and preferably the activity is increased non-targeted.

Accordingly, in one embodiment, the molecule which activity is to be increased in the process of the invention is the gene product with an activity as a “hydrolase”, preferably it is the molecule of section (a) or (b) of this paragraph.

In particular, it was observed that in plants, especially in Arabidopsis thaliana, increasing or generating the activity of a gene product non-targeted with the activity of a “hydrolase”, preferably being encoded by a gene comprising the nucleic acid sequence SEQ ID NO. 6510, preferably the coding region thereof, conferred the production of or the increase in phenylalanine compared with the wild type control.

The nucleic acid sequence of AvinDRAFT_{—}5103 from Azotobacter vinelandii, e.g. as shown in the respective line in column 5 of Table I, application no. 3, has been published in D. And the activity of the gene product thereof is the activity of hydrolase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “hydrolase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}5103, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}5103, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}5103, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}5103, and preferably the activity is increased non-targeted.

Accordingly, in one embodiment, the molecule which activity is to be increased in the process of the invention is the gene product with an activity as a “hydrolase”, preferably it is the molecule of section (a) or (b) of this paragraph.

In particular, it was observed that in plants, especially in Arabidopsis thaliana, increasing or generating the activity of a gene product non-targeted with the activity of a “hydrolase”, preferably being encoded by a gene comprising the nucleic acid sequence SEQ ID NO. 6510, preferably the coding region thereof, conferred the production of or the increase in tyrosine compared with the wild type control.

The nucleic acid sequence of AvinDRAFT_{—}5103 from Azotobacter vinelandii, e.g. as shown in the respective line in column 5 of Table I, application no. 3, has been published in D. And the activity of the gene product thereof is the activity of hydrolase.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “hydrolase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}5103, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}5103, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}5103, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}5103, and preferably the activity is increased non-targeted.

Accordingly, in one embodiment, the molecule which activity is to be increased in the process of the invention is the gene product with an activity as a “hydrolase”, preferably it is the molecule of section (a) or (b) of this paragraph.

In particular, it was observed that in plants, especially in Arabidopsis thaliana, increasing or generating the activity of a gene product non-targeted with the activity of a “hydrolase”, preferably being encoded by a gene comprising the nucleic acid sequence SEQ ID NO. 6510, preferably the coding region thereof, conferred the production of or the increase in 5-oxoproline compared with the wild type control.

The nucleic acid sequence of AvinDRAFT_{—}5246 from Azotobacter vinelandii, e.g. as shown in the respective line in column 5 of Table I, application no. 3, has been published in D. And the activity of the gene product thereof is the activity of glutamate-ammonia-ligase.

Accordingly, in one embodiment, the process of the present invention for producing aspartate in a non-human organism, like a microorganism or a plant or a part thereof, comprises increasing or generating the activity of a gene product with the activity of a gene product conferring the activity of “glutamate-ammonia-ligase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}5246, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}5246, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}5246, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}5246, and preferably the activity is increased non-targeted.

Accordingly, in one embodiment, the molecule which activity is to be increased in the process of the invention is the gene product with an activity as a “glutamate-ammonia-ligase”, preferably it is the molecule of section (a) or (b) of this paragraph.

In particular, it was observed that in plants, especially in Arabidopsis thaliana, increasing or generating the activity of a gene product non-targeted with the activity of a “glutamate-ammonia-ligase”, preferably being encoded by a gene comprising the nucleic acid sequence SEQ ID NO. 32037, preferably the coding region thereof, conferred the production of or the increase in aspartate compared with the wild type control.

The nucleic acid sequence of AvinDRAFT_{—}5246 from Azotobacter vinelandii, e.g. as shown in the respective line in column 5 of Table I, application no. 3, has been published in D. And the activity of the gene product thereof is the activity of glutamate-ammonia-ligase.

Accordingly, in one embodiment, the process of the present invention for producing serine in a non-human organism, like a microorganism or a plant or a part thereof, comprises increasing or generating the activity of a gene product with the activity of a gene product conferring the activity of “glutamate-ammonia-ligase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}5246, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}5246, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}5246, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}5246, and preferably the activity is increased non-targeted.

Accordingly, in one embodiment, the molecule which activity is to be increased in the process of the invention is the gene product with an activity as a “glutamate-ammonia-ligase”, preferably it is the molecule of section (a) or (b) of this paragraph.

In particular, it was observed that in plants, especially in Arabidopsis thaliana, increasing or generating the activity of a gene product non-targeted with the activity of a “glutamate-ammonia-ligase”, preferably being encoded by a gene comprising the nucleic acid sequence SEQ ID NO. 32037, preferably the coding region thereof, conferred the production of or the increase in serine compared with the wild type control.

The nucleic acid sequence of AvinDRAFT_{—}5246 from Azotobacter vinelandii, e.g. as shown in the respective line in column 5 of Table I, application no. 3, has been published in D. And the activity of the gene product thereof is the activity of glutamate-ammonia-ligase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “glutamate-ammonia-ligase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}5246, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}5246, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}5246, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}5246, and preferably the activity is increased non-targeted.

Accordingly, in one embodiment, the molecule which activity is to be increased in the process of the invention is the gene product with an activity as a “glutamate-ammonia-ligase”, preferably it is the molecule of section (a) or (b) of this paragraph.

In particular, it was observed that in plants, especially in Arabidopsis thaliana, increasing or generating the activity of a gene product non-targeted with the activity of a “glutamate-ammonia-ligase”, preferably being encoded by a gene comprising the nucleic acid sequence SEQ ID NO. 32037, preferably the coding region thereof, conferred the production of or the increase in tyrosine compared with the wild type control.

The nucleic acid sequence of AvinDRAFT_{—}5292 from Azotobacter vinelandii, e.g. as shown in the respective line in column 5 of Table I, application no. 3, has been published in D. And the activity of the gene product thereof is the activity of fumarylacetoacetate hydrolase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “fumarylacetoacetate hydrolase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}5292, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}5292, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}5292, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}5292, and preferably the activity is increased non-targeted.

Accordingly, in one embodiment, the molecule which activity is to be increased in the process of the invention is the gene product with an activity as a “fumarylacetoacetate hydrolase”, preferably it is the molecule of section (a) or (b) of this paragraph.

In particular, it was observed that in plants, especially in Arabidopsis thaliana, increasing or generating the activity of a gene product non-targeted with the activity of a “fumarylacetoacetate hydrolase”, preferably being encoded by a gene comprising the nucleic acid sequence SEQ ID NO. 32308, preferably the coding region thereof, conferred the production of or the increase in phenylalanine compared with the wild type control.

The nucleic acid sequence of AvinDRAFT_{—}5292 from Azotobacter vinelandii, e.g. as shown in the respective line in column 5 of Table I, application no. 3, has been published in D. And the activity of the gene product thereof is the activity of fumarylacetoacetate hydrolase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “fumarylacetoacetate hydrolase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}5292, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}5292, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}5292, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}5292, and preferably the activity is increased non-targeted.

Accordingly, in one embodiment, the molecule which activity is to be increased in the process of the invention is the gene product with an activity as a “fumarylacetoacetate hydrolase”, preferably it is the molecule of section (a) or (b) of this paragraph.

In particular, it was observed that in plants, especially in Arabidopsis thaliana, increasing or generating the activity of a gene product non-targeted with the activity of a “fumarylacetoacetate hydrolase”, preferably being encoded by a gene comprising the nucleic acid sequence SEQ ID NO. 32308, preferably the coding region thereof, conferred the production of or the increase in tyrosine compared with the wild type control.

The nucleic acid sequence of AvinDRAFT_{—}5467 from Azotobacter vinelandii, e.g. as shown in the respective line in column 5 of Table I, application no. 3, has been published in D. And the activity of the gene product thereof is the activity of HesB/YadR/YfhF family protein.

Accordingly, in one embodiment, the process of the present invention for producing serine in a non-human organism, like a microorganism or a plant or a part thereof, comprises increasing or generating the activity of a gene product with the activity of a gene product conferring the activity of “HesB/YadR/YfhF family protein”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}5467, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}5467, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}5467, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}5467, and preferably the activity is increased non-targeted.

Accordingly, in one embodiment, the molecule which activity is to be increased in the process of the invention is the gene product with an activity as a “HesB/YadR/YfhF family protein”, preferably it is the molecule of section (a) or (b) of this paragraph.

In particular, it was observed that in plants, especially in Arabidopsis thaliana, increasing or generating the activity of a gene product non-targeted with the activity of a “HesB/YadR/YfhF family protein”, preferably being encoded by a gene comprising the nucleic acid sequence SEQ ID NO. 32648, preferably the coding region thereof, conferred the production of or the increase in serine compared with the wild type control.

The nucleic acid sequence of AvinDRAFT_{—}5579 from Azotobacter vinelandii, e.g. as shown in the respective line in column 5 of Table I, application no. 3, has been published in D. And the activity of the gene product thereof is the activity of peptidase.

Accordingly, in one embodiment, the process of the present invention for producing aspartate in a non-human organism, like a microorganism or a plant or a part thereof, comprises increasing or generating the activity of a gene product with the activity of a gene product conferring the activity of “peptidase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}5579, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}5579, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}5579, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}5579, and preferably the activity is increased non-targeted.

Accordingly, in one embodiment, the molecule which activity is to be increased in the process of the invention is the gene product with an activity as a “peptidase”, preferably it is the molecule of section (a) or (b) of this paragraph.

In particular, it was observed that in plants, especially in Arabidopsis thaliana, increasing or generating the activity of a gene product non-targeted with the activity of a “peptidase”, preferably being encoded by a gene comprising the nucleic acid sequence SEQ ID NO. 74662, preferably the coding region thereof, conferred the production of or the increase in aspartate compared with the wild type control.

The nucleic acid sequence of AvinDRAFT_{—}5644 from Azotobacter vinelandii, e.g. as shown in the respective line in column 5 of Table I, application no. 3, has been published in D. And the activity of the gene product thereof is the activity of L-aspartate oxidase.

Accordingly, in one embodiment, the process of the present invention for producing aspartate in a non-human organism, like a microorganism or a plant or a part thereof, comprises increasing or generating the activity of a gene product with the activity of a gene product conferring the activity of “L-aspartate oxidase”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}5644, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}5644, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}5644, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}5644, and preferably the activity is increased non-targeted.

Accordingly, in one embodiment, the molecule which activity is to be increased in the process of the invention is the gene product with an activity as a “L-aspartate oxidase”, preferably it is the molecule of section (a) or (b) of this paragraph.

In particular, it was observed that in plants, especially in Arabidopsis thaliana, increasing or generating the activity of a gene product non-targeted with the activity of a “L-aspartate oxidase”, preferably being encoded by a gene comprising the nucleic acid sequence SEQ ID NO. 33085, preferably the coding region thereof, conferred the production of or the increase in aspartate compared with the wild type control.

The nucleic acid sequence of AvinDRAFT_{—}6093 from Azotobacter vinelandii, e.g. as shown in the respective line in column 5 of Table I, application no. 3, has been published in D. And the activity of the gene product thereof is the activity of Chaperone protein CIpB.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “Chaperone protein CIpB”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}6093, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}6093, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}6093, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}6093, and preferably the activity is increased non-targeted.

Accordingly, in one embodiment, the molecule which activity is to be increased in the process of the invention is the gene product with an activity as a “Chaperone protein CIpB”, preferably it is the molecule of section (a) or (b) of this paragraph.

In particular, it was observed that in plants, especially in Arabidopsis thaliana, increasing or generating the activity of a gene product non-targeted with the activity of a “Chaperone protein CIpB”, preferably being encoded by a gene comprising the nucleic acid sequence SEQ ID NO. 33596, preferably the coding region thereof, conferred the production of or the increase in phenylalanine compared with the wild type control.

The nucleic acid sequence of AvinDRAFT_{—}6679 from Azotobacter vinelandii, e.g. as shown in the respective line in column 5 of Table I, application no. 3, has been published in D. And the activity of the gene product thereof is the activity of electron transfer flavoprotein subunit beta.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “electron transfer flavoprotein subunit beta”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}6679, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}6679, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}6679, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}6679, and preferably the activity is increased non-targeted.

Accordingly, in one embodiment, the molecule which activity is to be increased in the process of the invention is the gene product with an activity as a “electron transfer flavoprotein subunit beta”, preferably it is the molecule of section (a) or (b) of this paragraph.

In particular, it was observed that in plants, especially in Arabidopsis thaliana, increasing or generating the activity of a gene product non-targeted with the activity of a “electron transfer flavoprotein subunit beta”, preferably being encoded by a gene comprising the nucleic acid sequence SEQ ID NO. 74729, preferably the coding region thereof, conferred the production of or the increase in phenylalanine compared with the wild type control.

The nucleic acid sequence of AvinDRAFT_{—}6679 from Azotobacter vinelandii, e.g. as shown in the respective line in column 5 of Table I, application no. 3, has been published in D. And the activity of the gene product thereof is the activity of electron transfer flavoprotein subunit beta.

Accordingly, in one embodiment, the process of the present invention for producing tyrosine in a non-human organism, like a microorganism or a plant or a part thereof, comprises increasing or generating the activity of a gene product with the activity of a gene product conferring the activity of “electron transfer flavoprotein subunit beta”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AvinDRAFT_{—}6679, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AvinDRAFT_{—}6679, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AvinDRAFT_{—}6679, or a functional equivalent or a homolog thereof as depicted in column 8 of Table II, application no. 3, preferably a homolog or functional equivalent as depicted in column 8 of Table II B, application no. 3, and being depicted in the same respective line as said AvinDRAFT_{—}6679, and preferably the activity is increased non-targeted.

Accordingly, in one embodiment, the molecule which activity is to be increased in the process of the invention is the gene product with an activity as a “electron transfer flavoprotein subunit beta”, preferably it is the molecule of section (a) or (b) of this paragraph.

In particular, it was observed that in plants, especially in Arabidopsis thaliana, increasing or generating the activity of a gene product non-targeted with the activity of a “electron transfer flavoprotein subunit beta”, preferably being encoded by a gene comprising the nucleic acid sequence SEQ ID NO. 74729, preferably the coding region thereof, conferred the production of or the increase in tyrosine compared with the wild type control.

The nucleic acid sequence of AX653549 from Oryza sativa, e.g. as shown in the respective line in column 5 of Table I, application no. 3, has been published in G. And the activity of the gene product thereof is the activity of AX653549-protein.

Accordingly, in one embodiment, the process of the present invention for producing citrulline in a non-human organism, like a microorganism or a plant or a part thereof, comprises increasing or generating the activity of a gene product with the activity of a gene product conferring the activity of “AX653549-protein”, especially from Oryza sativa or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AX653549, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AX653549, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AX653549, or a functional equivalent or a homolog thereof as depicted in column 8 of Table II, application no. 3, preferably a homolog or functional equivalent as depicted in column 8 of Table II B, application no. 3, and being depicted in the same respective line as said AX653549, and preferably the activity is increased non-targeted.

Accordingly, in one embodiment, the molecule which activity is to be increased in the process of the invention is the gene product with an activity as a “AX653549-protein”, preferably it is the molecule of section (a) or (b) of this paragraph.

In particular, it was observed that in plants, especially in Arabidopsis thaliana, increasing or generating the activity of a gene product non-targeted with the activity of a “AX653549-protein”, preferably being encoded by a gene comprising the nucleic acid sequence SEQ ID NO. 34301, preferably the coding region thereof, conferred the production of or the increase in citrulline compared with the wild type control.

The nucleic acid sequence of AX653549 from Oryza sativa, e.g. as shown in the respective line in column 5 of Table I, application no. 3, has been published in G. And the activity of the gene product thereof is the activity of AX653549-protein.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “AX653549-protein”, especially from Oryza sativa or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AX653549, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AX653549, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AX653549, or a functional equivalent or a homolog thereof as depicted in column 8 of Table II, application no. 3, preferably a homolog or functional equivalent as depicted in column 8 of Table II B, application no. 3, and being depicted in the same respective line as said AX653549, and preferably the activity is increased non-targeted.

Accordingly, in one embodiment, the molecule which activity is to be increased in the process of the invention is the gene product with an activity as a “AX653549-protein”, preferably it is the molecule of section (a) or (b) of this paragraph.

In particular, it was observed that in plants, especially in Arabidopsis thaliana, increasing or generating the activity of a gene product non-targeted with the activity of a “AX653549-protein”, preferably being encoded by a gene comprising the nucleic acid sequence SEQ ID NO. 34301, preferably the coding region thereof, conferred the production of or the increase in 5-oxoproline compared with the wild type control.

The nucleic acid sequence of AY087308 from Arabidopsis thaliana, e.g. as shown in the respective line in column 5 of Table I, application no. 3, has been published in F. And the activity of the gene product thereof is the activity of AY087308-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “AY087308-protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AY087308, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AY087308, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AY087308, or a functional equivalent or a homolog thereof as depicted in column 8 of Table II, application no. 3, preferably a homolog or functional equivalent as depicted in column 8 of Table II B, application no. 3, and being depicted in the same respective line as said AY087308, and preferably the activity is increased non-targeted.

Accordingly, in one embodiment, the molecule which activity is to be increased in the process of the invention is the gene product with an activity as a “AY087308-protein”, preferably it is the molecule of section (a) or (b) of this paragraph.

In particular, it was observed that in plants, especially in Arabidopsis thaliana, increasing or generating the activity of a gene product non-targeted with the activity of a “AY087308-protein”, preferably being encoded by a gene comprising the nucleic acid sequence SEQ ID NO. 34602, preferably the coding region thereof, conferred the production of or the increase in aspartate compared with the wild type control.

The nucleic acid sequence of AY087308 from Arabidopsis thaliana, e.g. as shown in the respective line in column 5 of Table I, application no. 3, has been published in F. And the activity of the gene product thereof is the activity of AY087308-protein.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “AY087308-protein”, especially from Arabidopsis thaliana or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said AY087308, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said AY087308, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said AY087308, or a functional equivalent or a homolog thereof as depicted in column 8 of Table II, application no. 3, preferably a homolog or functional equivalent as depicted in column 8 of Table II B, application no. 3, and being depicted in the same respective line as said AY087308, and preferably the activity is increased non-targeted.

Accordingly, in one embodiment, the molecule which activity is to be increased in the process of the invention is the gene product with an activity as a “AY087308-protein”, preferably it is the molecule of section (a) or (b) of this paragraph.

In particular, it was observed that in plants, especially in Arabidopsis thaliana, increasing or generating the activity of a gene product non-targeted with the activity of a “AY087308-protein”, preferably being encoded by a gene comprising the nucleic acid sequence SEQ ID NO. 34602, preferably the coding region thereof, conferred the production of or the increase in 5-oxoproline compared with the wild type control.

The nucleic acid sequence of B0004 from Escherichia coli, e.g. as shown in the respective line in column 5 of Table I, application no. 3, has been published in B. And the activity of the gene product thereof is the activity of threonine synthase.

Accordingly, in one embodiment, the process of the present invention for producing alanine in a non-human organism, like a microorganism or a plant or a part thereof, comprises increasing or generating the activity of a gene product with the activity of a gene product conferring the activity of “threonine synthase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0004, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0004, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0004, or a functional equivalent or a homolog thereof as depicted in column 8 of Table II, application no. 3, preferably a homolog or functional equivalent as depicted in column 8 of Table II B, application no. 3, and being depicted in the same respective line as said B0004, and preferably the activity is increased plastidic.

Accordingly, in one embodiment, the molecule which activity is to be increased in the process of the invention is the gene product with an activity as a “threonine synthase”, preferably it is the molecule of section (a) or (b) of this paragraph.

In particular, it was observed that in plants, especially in Arabidopsis thaliana, increasing or generating the activity of a gene product plastidic with the activity of a “threonine synthase”, preferably being encoded by a gene comprising the nucleic acid sequence SEQ ID NO. 34889, preferably the coding region thereof, conferred the production of or the increase in alanine compared with the wild type control.

The nucleic acid sequence of B0004 from Escherichia coli, e.g. as shown in the respective line in column 5 of Table I, application no. 3, has been published in B. And the activity of the gene product thereof is the activity of threonine synthase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “threonine synthase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0004, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0004, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0004, or a functional equivalent or a homolog thereof as depicted in column 8 of Table II, application no. 3, preferably a homolog or functional equivalent as depicted in column 8 of Table II B, application no. 3, and being depicted in the same respective line as said B0004, and preferably the activity is increased plastidic.

Accordingly, in one embodiment, the molecule which activity is to be increased in the process of the invention is the gene product with an activity as a “threonine synthase”, preferably it is the molecule of section (a) or (b) of this paragraph.

In particular, it was observed that in plants, especially in Arabidopsis thaliana, increasing or generating the activity of a gene product plastidic with the activity of a “threonine synthase”, preferably being encoded by a gene comprising the nucleic acid sequence SEQ ID NO. 34889, preferably the coding region thereof, conferred the production of or the increase in phenylalanine compared with the wild type control.

The nucleic acid sequence of B0004 from Escherichia coli, e.g. as shown in the respective line in column 5 of Table I, application no. 3, has been published in B. And the activity of the gene product thereof is the activity of threonine synthase.

Accordingly, in one embodiment, the process of the present invention for producing serine in a non-human organism, like a microorganism or a plant or a part thereof, comprises increasing or generating the activity of a gene product with the activity of a gene product conferring the activity of “threonine synthase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0004, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0004, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0004, or a functional equivalent or a homolog thereof as depicted in column 8 of Table II, application no. 3, preferably a homolog or functional equivalent as depicted in column 8 of Table II B, application no. 3, and being depicted in the same respective line as said B0004, and preferably the activity is increased plastidic.

Accordingly, in one embodiment, the molecule which activity is to be increased in the process of the invention is the gene product with an activity as a “threonine synthase”, preferably it is the molecule of section (a) or (b) of this paragraph.

In particular, it was observed that in plants, especially in Arabidopsis thaliana, increasing or generating the activity of a gene product plastidic with the activity of a “threonine synthase”, preferably being encoded by a gene comprising the nucleic acid sequence SEQ ID NO. 34889, preferably the coding region thereof, conferred the production of or the increase in serine compared with the wild type control.

The nucleic acid sequence of B0004 from Escherichia coli, e.g. as shown in the respective line in column 5 of Table I, application no. 3, has been published in B. And the activity of the gene product thereof is the activity of threonine synthase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “threonine synthase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0004, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0004, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0004, or a functional equivalent or a homolog thereof as depicted in column 8 of Table II, application no. 3, preferably a homolog or functional equivalent as depicted in column 8 of Table II B, application no. 3, and being depicted in the same respective line as said B0004, and preferably the activity is increased plastidic.

Accordingly, in one embodiment, the molecule which activity is to be increased in the process of the invention is the gene product with an activity as a “threonine synthase”, preferably it is the molecule of section (a) or (b) of this paragraph.

In particular, it was observed that in plants, especially in Arabidopsis thaliana, increasing or generating the activity of a gene product plastidic with the activity of a “threonine synthase”, preferably being encoded by a gene comprising the nucleic acid sequence SEQ ID NO. 34889, preferably the coding region thereof, conferred the production of or the increase in tyrosine compared with the wild type control.

The nucleic acid sequence of B0004 from Escherichia coli, e.g. as shown in the respective line in column 5 of Table I, application no. 3, has been published in B. And the activity of the gene product thereof is the activity of threonine synthase.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “threonine synthase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0004, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0004, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0004, or a functional equivalent or a homolog thereof as depicted in column 8 of Table II, application no. 3, preferably a homolog or functional equivalent as depicted in column 8 of Table II B, application no. 3, and being depicted in the same respective line as said B0004, and preferably the activity is increased plastidic.

Accordingly, in one embodiment, the molecule which activity is to be increased in the process of the invention is the gene product with an activity as a “threonine synthase”, preferably it is the molecule of section (a) or (b) of this paragraph.

In particular, it was observed that in plants, especially in Arabidopsis thaliana, increasing or generating the activity of a gene product plastidic with the activity of a “threonine synthase”, preferably being encoded by a gene comprising the nucleic acid sequence SEQ ID NO. 34889, preferably the coding region thereof, conferred the production of or the increase in 5-oxoproline compared with the wild type control.

The nucleic acid sequence of B0078 from Escherichia coli, e.g. as shown in the respective line in column 5 of Table I, application no. 3, has been published in B. And the activity of the gene product thereof is the activity of acetolactate synthase small subunit.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “acetolactate synthase small subunit”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0078, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0078, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0078, or a functional equivalent or a homolog thereof as depicted in column 8 of Table II, application no. 3, preferably a homolog or functional equivalent as depicted in column 8 of Table II B, application no. 3, and being depicted in the same respective line as said B0078, and preferably the activity is increased non-targeted.

Accordingly, in one embodiment, the molecule which activity is to be increased in the process of the invention is the gene product with an activity as a “acetolactate synthase small subunit”, preferably it is the molecule of section (a) or (b) of this paragraph.

In particular, it was observed that in plants, especially in Arabidopsis thaliana, increasing or generating the activity of a gene product non-targeted with the activity of a “acetolactate synthase small subunit”, preferably being encoded by a gene comprising the nucleic acid sequence SEQ ID NO. 6818, preferably the coding region thereof, conferred the production of or the increase in phenylalanine compared with the wild type control.

The nucleic acid sequence of B0124 from Escherichia coli, e.g. as shown in the respective line in column 5 of Table I, application no. 3, has been published in B. And the activity of the gene product thereof is the activity of glucose dehydrogenase.

Accordingly, in one embodiment, the process of the present invention for producing glycine in a non-human organism, like a microorganism or a plant or a part thereof, comprises increasing or generating the activity of a gene product with the activity of a gene product conferring the activity of “glucose dehydrogenase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0124, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0124, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0124, or a functional equivalent or a homolog thereof as depicted in column 8 of Table II, application no. 3, preferably a homolog or functional equivalent as depicted in column 8 of Table II B, application no. 3, and being depicted in the same respective line as said B0124, and preferably the activity is increased non-targeted.

Accordingly, in one embodiment, the molecule which activity is to be increased in the process of the invention is the gene product with an activity as a “glucose dehydrogenase”, preferably it is the molecule of section (a) or (b) of this paragraph.

In particular, it was observed that in plants, especially in Arabidopsis thaliana, increasing or generating the activity of a gene product non-targeted with the activity of a “glucose dehydrogenase”, preferably being encoded by a gene comprising the nucleic acid sequence SEQ ID NO. 35482, preferably the coding region thereof, conferred the production of or the increase in glycine compared with the wild type control.

The nucleic acid sequence of B0124 from Escherichia coli, e.g. as shown in the respective line in column 5 of Table I, application no. 3, has been published in B. And the activity of the gene product thereof is the activity of glucose dehydrogenase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “glucose dehydrogenase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0124, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0124, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0124, or a functional equivalent or a homolog thereof as depicted in column 8 of Table II, application no. 3, preferably a homolog or functional equivalent as depicted in column 8 of Table II B, application no. 3, and being depicted in the same respective line as said B0124, and preferably the activity is increased non-targeted.

Accordingly, in one embodiment, the molecule which activity is to be increased in the process of the invention is the gene product with an activity as a “glucose dehydrogenase”, preferably it is the molecule of section (a) or (b) of this paragraph.

In particular, it was observed that in plants, especially in Arabidopsis thaliana, increasing or generating the activity of a gene product non-targeted with the activity of a “glucose dehydrogenase”, preferably being encoded by a gene comprising the nucleic acid sequence SEQ ID NO. 35482, preferably the coding region thereof, conferred the production of or the increase in phenylalanine compared with the wild type control.

The nucleic acid sequence of B0124 from Escherichia coli, e.g. as shown in the respective line in column 5 of Table I, application no. 3, has been published in B. And the activity of the gene product thereof is the activity of glucose dehydrogenase.

Accordingly, in one embodiment, the process of the present invention for producing serine in a non-human organism, like a microorganism or a plant or a part thereof, comprises increasing or generating the activity of a gene product with the activity of a gene product conferring the activity of “glucose dehydrogenase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0124, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0124, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0124, or a functional equivalent or a homolog thereof as depicted in column 8 of Table II, application no. 3, preferably a homolog or functional equivalent as depicted in column 8 of Table II B, application no. 3, and being depicted in the same respective line as said B0124, and preferably the activity is increased non-targeted.

Accordingly, in one embodiment, the molecule which activity is to be increased in the process of the invention is the gene product with an activity as a “glucose dehydrogenase”, preferably it is the molecule of section (a) or (b) of this paragraph.

In particular, it was observed that in plants, especially in Arabidopsis thaliana, increasing or generating the activity of a gene product non-targeted with the activity of a “glucose dehydrogenase”, preferably being encoded by a gene comprising the nucleic acid sequence SEQ ID NO. 35482, preferably the coding region thereof, conferred the production of or the increase in serine compared with the wild type control.

The nucleic acid sequence of B0124 from Escherichia coli, e.g. as shown in the respective line in column 5 of Table I, application no. 3, has been published in B. And the activity of the gene product thereof is the activity of glucose dehydrogenase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “glucose dehydrogenase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0124, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0124, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0124, or a functional equivalent or a homolog thereof as depicted in column 8 of Table II, application no. 3, preferably a homolog or functional equivalent as depicted in column 8 of Table II B, application no. 3, and being depicted in the same respective line as said B0124, and preferably the activity is increased non-targeted.

Accordingly, in one embodiment, the molecule which activity is to be increased in the process of the invention is the gene product with an activity as a “glucose dehydrogenase”, preferably it is the molecule of section (a) or (b) of this paragraph.

In particular, it was observed that in plants, especially in Arabidopsis thaliana, increasing or generating the activity of a gene product non-targeted with the activity of a “glucose dehydrogenase”, preferably being encoded by a gene comprising the nucleic acid sequence SEQ ID NO. 35482, preferably the coding region thereof, conferred the production of or the increase in tyrosine compared with the wild type control.

The nucleic acid sequence of B0131 from Escherichia coli, e.g. as shown in the respective line in column 5 of Table I, application no. 3, has been published in B. And the activity of the gene product thereof is the activity of aspartate 1-decarboxylase.

Accordingly, in one embodiment, the process of the present invention for producing asparagine in a non-human organism, like a microorganism or a plant or a part thereof, comprises increasing or generating the activity of a gene product with the activity of a gene product conferring the activity of “aspartate 1-decarboxylase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0131, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0131, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0131, or a functional equivalent or a homolog thereof as depicted in column 8 of Table II, application no. 3, preferably a homolog or functional equivalent as depicted in column 8 of Table II B, application no. 3, and being depicted in the same respective line as said B0131, and preferably the activity is increased non-targeted.

Accordingly, in one embodiment, the molecule which activity is to be increased in the process of the invention is the gene product with an activity as a “aspartate 1-decarboxylase”, preferably it is the molecule of section (a) or (b) of this paragraph.

In particular, it was observed that in plants, especially in Arabidopsis thaliana, increasing or generating the activity of a gene product non-targeted with the activity of a “aspartate 1-decarboxylase”, preferably being encoded by a gene comprising the nucleic acid sequence SEQ ID NO. 74986, preferably the coding region thereof, conferred the production of or the increase in asparagine compared with the wild type control.

The nucleic acid sequence of B0131 from Escherichia coli, e.g. as shown in the respective line in column 5 of Table I, application no. 3, has been published in B. And the activity of the gene product thereof is the activity of aspartate 1-decarboxylase.

Accordingly, in one embodiment, the process of the present invention for producing serine in a non-human organism, like a microorganism or a plant or a part thereof, comprises increasing or generating the activity of a gene product with the activity of a gene product conferring the activity of “aspartate 1-decarboxylase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0131, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0131, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0131, or a functional equivalent or a homolog thereof as depicted in column 8 of Table II, application no. 3, preferably a homolog or functional equivalent as depicted in column 8 of Table II B, application no. 3, and being depicted in the same respective line as said B0131, and preferably the activity is increased non-targeted.

Accordingly, in one embodiment, the molecule which activity is to be increased in the process of the invention is the gene product with an activity as a “aspartate 1-decarboxylase”, preferably it is the molecule of section (a) or (b) of this paragraph.

In particular, it was observed that in plants, especially in Arabidopsis thaliana, increasing or generating the activity of a gene product non-targeted with the activity of a “aspartate 1-decarboxylase”, preferably being encoded by a gene comprising the nucleic acid sequence SEQ ID NO. 74986, preferably the coding region thereof, conferred the production of or the increase in serine compared with the wild type control.

The nucleic acid sequence of B0161 from Escherichia coli, e.g. as shown in the respective line in column 5 of Table I, application no. 3, has been published in B. And the activity of the gene product thereof is the activity of serine protease.

Accordingly, in one embodiment, the process of the present invention for producing alanine in a non-human organism, like a microorganism or a plant or a part thereof, comprises increasing or generating the activity of a gene product with the activity of a gene product conferring the activity of “serine protease”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0161, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0161, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0161, or a functional equivalent or a homolog thereof as depicted in column 8 of Table II, application no. 3, preferably a homolog or functional equivalent as depicted in column 8 of Table II B, application no. 3, and being depicted in the same respective line as said B0161, and preferably the activity is increased non-targeted.

Accordingly, in one embodiment, the molecule which activity is to be increased in the process of the invention is the gene product with an activity as a “serine protease”, preferably it is the molecule of section (a) or (b) of this paragraph.

In particular, it was observed that in plants, especially in Arabidopsis thaliana, increasing or generating the activity of a gene product non-targeted with the activity of a “serine protease”, preferably being encoded by a gene comprising the nucleic acid sequence SEQ ID NO. 7081, preferably the coding region thereof, conferred the production of or the increase in alanine compared with the wild type control.

The nucleic acid sequence of B0161 from Escherichia coli, e.g. as shown in the respective line in column 5 of Table I, application no. 3, has been published in B. And the activity of the gene product thereof is the activity of serine protease.

Accordingly, in one embodiment, the process of the present invention for producing serine in a non-human organism, like a microorganism or a plant or a part thereof, comprises increasing or generating the activity of a gene product with the activity of a gene product conferring the activity of “serine protease”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0161, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0161, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0161, or a functional equivalent or a homolog thereof as depicted in column 8 of Table II, application no. 3, preferably a homolog or functional equivalent as depicted in column 8 of Table II B, application no. 3, and being depicted in the same respective line as said B0161, and preferably the activity is increased non-targeted.

Accordingly, in one embodiment, the molecule which activity is to be increased in the process of the invention is the gene product with an activity as a “serine protease”, preferably it is the molecule of section (a) or (b) of this paragraph.

In particular, it was observed that in plants, especially in Arabidopsis thaliana, increasing or generating the activity of a gene product non-targeted with the activity of a “serine protease”, preferably being encoded by a gene comprising the nucleic acid sequence SEQ ID NO. 7081, preferably the coding region thereof, conferred the production of or the increase in serine compared with the wild type control.

The nucleic acid sequence of B0161 from Escherichia coli, e.g. as shown in the respective line in column 5 of Table I, application no. 3, has been published in B. And the activity of the gene product thereof is the activity of serine protease.

Accordingly, in one embodiment, the process of the present invention for producing tyrosine in a non-human organism, like a microorganism or a plant or a part thereof, comprises increasing or generating the activity of a gene product with the activity of a gene product conferring the activity of “serine protease”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0161, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0161, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0161, or a functional equivalent or a homolog thereof as depicted in column 8 of Table II, application no. 3, preferably a homolog or functional equivalent as depicted in column 8 of Table II B, application no. 3, and being depicted in the same respective line as said B0161, and preferably the activity is increased non-targeted.

Accordingly, in one embodiment, the molecule which activity is to be increased in the process of the invention is the gene product with an activity as a “serine protease”, preferably it is the molecule of section (a) or (b) of this paragraph.

In particular, it was observed that in plants, especially in Arabidopsis thaliana, increasing or generating the activity of a gene product non-targeted with the activity of a “serine protease”, preferably being encoded by a gene comprising the nucleic acid sequence SEQ ID NO. 7081, preferably the coding region thereof, conferred the production of or the increase in tyrosine compared with the wild type control.

The nucleic acid sequence of B0221 from Escherichia coli, e.g. as shown in the respective line in column 5 of Table I, application no. 3, has been published in B. And the activity of the gene product thereof is the activity of acyl-CoA dehydrogenase.

Accordingly, in one embodiment, the process of the present invention for producing serine in a non-human organism, like a microorganism or a plant or a part thereof, comprises increasing or generating the activity of a gene product with the activity of a gene product conferring the activity of “acyl-CoA dehydrogenase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0221, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0221, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0221, or a functional equivalent or a homolog thereof as depicted in column 8 of Table II, application no. 3, preferably a homolog or functional equivalent as depicted in column 8 of Table II B, application no. 3, and being depicted in the same respective line as said B0221, and preferably the activity is increased non-targeted.

Accordingly, in one embodiment, the molecule which activity is to be increased in the process of the invention is the gene product with an activity as a “acyl-CoA dehydrogenase”, preferably it is the molecule of section (a) or (b) of this paragraph.

In particular, it was observed that in plants, especially in Arabidopsis thaliana, increasing or generating the activity of a gene product non-targeted with the activity of a “acyl-CoA dehydrogenase”, preferably being encoded by a gene comprising the nucleic acid sequence SEQ ID NO. 35590, preferably the coding region thereof, conferred the production of or the increase in serine compared with the wild type control.

The nucleic acid sequence of B0221 from Escherichia coli, e.g. as shown in the respective line in column 5 of Table I, application no. 3, has been published in B. And the activity of the gene product thereof is the activity of acyl-CoA dehydrogenase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “acyl-CoA dehydrogenase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0221, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0221, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0221, or a functional equivalent or a homolog thereof as depicted in column 8 of Table II, application no. 3, preferably a homolog or functional equivalent as depicted in column 8 of Table II B, application no. 3, and being depicted in the same respective line as said B0221, and preferably the activity is increased non-targeted.

Accordingly, in one embodiment, the molecule which activity is to be increased in the process of the invention is the gene product with an activity as a “acyl-CoA dehydrogenase”, preferably it is the molecule of section (a) or (b) of this paragraph.

In particular, it was observed that in plants, especially in Arabidopsis thaliana, increasing or generating the activity of a gene product non-targeted with the activity of a “acyl-CoA dehydrogenase”, preferably being encoded by a gene comprising the nucleic acid sequence SEQ ID NO. 35590, preferably the coding region thereof, conferred the production of or the increase in tyrosine compared with the wild type control.

The nucleic acid sequence of B0344 from Escherichia coli, e.g. as shown in the respective line in column 5 of Table I, application no. 3, has been published in B. And the activity of the gene product thereof is the activity of beta-galactosidase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “beta-galactosidase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0344, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0344, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0344, or a functional equivalent or a homolog thereof as depicted in column 8 of Table II, application no. 3, preferably a homolog or functional equivalent as depicted in column 8 of Table II B, application no. 3, and being depicted in the same respective line as said B0344, and preferably the activity is increased plastidic.

Accordingly, in one embodiment, the molecule which activity is to be increased in the process of the invention is the gene product with an activity as a “beta-galactosidase”, preferably it is the molecule of section (a) or (b) of this paragraph.

In particular, it was observed that in plants, especially in Arabidopsis thaliana, increasing or generating the activity of a gene product plastidic with the activity of a “beta-galactosidase”, preferably being encoded by a gene comprising the nucleic acid sequence SEQ ID NO. 35733, preferably the coding region thereof, conferred the production of or the increase in tyrosine compared with the wild type control.

The nucleic acid sequence of B0344 from Escherichia coli, e.g. as shown in the respective line in column 5 of Table I, application no. 3, has been published in B. And the activity of the gene product thereof is the activity of beta-galactosidase.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “beta-galactosidase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0344, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0344, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0344, or a functional equivalent or a homolog thereof as depicted in column 8 of Table II, application no. 3, preferably a homolog or functional equivalent as depicted in column 8 of Table II B, application no. 3, and being depicted in the same respective line as said B0344, and preferably the activity is increased plastidic.

Accordingly, in one embodiment, the molecule which activity is to be increased in the process of the invention is the gene product with an activity as a “beta-galactosidase”, preferably it is the molecule of section (a) or (b) of this paragraph.

In particular, it was observed that in plants, especially in Arabidopsis thaliana, increasing or generating the activity of a gene product plastidic with the activity of a “beta-galactosidase”, preferably being encoded by a gene comprising the nucleic acid sequence SEQ ID NO. 35733, preferably the coding region thereof, conferred the production of or the increase in 5-oxoproline compared with the wild type control.

The nucleic acid sequence of B0348 from Escherichia coli, e.g. as shown in the respective line in column 5 of Table I, application no. 3, has been published in B. And the activity of the gene product thereof is the activity of 2,3-dihydroxyphenylpropionate 1,2-dioxygenase.

Accordingly, in one embodiment, the process of the present invention for producing glycine in a non-human organism, like a microorganism or a plant or a part thereof, comprises increasing or generating the activity of a gene product with the activity of a gene product conferring the activity of “2,3-dihydroxyphenylpropionate 1,2-dioxygenase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0348, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0348, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0348, or a functional equivalent or a homolog thereof as depicted in column 8 of Table II, application no. 3, preferably a homolog or functional equivalent as depicted in column 8 of Table II B, application no. 3, and being depicted in the same respective line as said B0348, and preferably the activity is increased plastidic.

Accordingly, in one embodiment, the molecule which activity is to be increased in the process of the invention is the gene product with an activity as a “2,3-dihydroxyphenylpropionate 1,2-dioxygenase”, preferably it is the molecule of section (a) or (b) of this paragraph.

In particular, it was observed that in plants, especially in Arabidopsis thaliana, increasing or generating the activity of a gene product plastidic with the activity of a “2,3-dihydroxyphenylpropionate 1,2-dioxygenase”, preferably being encoded by a gene comprising the nucleic acid sequence SEQ ID NO. 7269, preferably the coding region thereof, conferred the production of or the increase in glycine compared with the wild type control.

The nucleic acid sequence of B0348 from Escherichia coli, e.g. as shown in the respective line in column 5 of Table I, application no. 3, has been published in B. And the activity of the gene product thereof is the activity of 2,3-dihydroxyphenylpropionate 1,2-dioxygenase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “2,3-dihydroxyphenylpropionate 1,2-dioxygenase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0348, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0348, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0348, or a functional equivalent or a homolog thereof as depicted in column 8 of Table II, application no. 3, preferably a homolog or functional equivalent as depicted in column 8 of Table II B, application no. 3, and being depicted in the same respective line as said B0348, and preferably the activity is increased plastidic.

Accordingly, in one embodiment, the molecule which activity is to be increased in the process of the invention is the gene product with an activity as a “2,3-dihydroxyphenylpropionate 1,2-dioxygenase”, preferably it is the molecule of section (a) or (b) of this paragraph.

In particular, it was observed that in plants, especially in Arabidopsis thaliana, increasing or generating the activity of a gene product plastidic with the activity of a “2,3-dihydroxyphenylpropionate 1,2-dioxygenase”, preferably being encoded by a gene comprising the nucleic acid sequence SEQ ID NO. 7269, preferably the coding region thereof, conferred the production of or the increase in phenylalanine compared with the wild type control.

The nucleic acid sequence of B0348 from Escherichia coli, e.g. as shown in the respective line in column 5 of Table I, application no. 3, has been published in B. And the activity of the gene product thereof is the activity of 2,3-dihydroxyphenylpropionate 1,2-dioxygenase.

Accordingly, in one embodiment, the process of the present invention for producing serine in a non-human organism, like a microorganism or a plant or a part thereof, comprises increasing or generating the activity of a gene product with the activity of a gene product conferring the activity of “2,3-dihydroxyphenylpropionate 1,2-dioxygenase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0348, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0348, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0348, or a functional equivalent or a homolog thereof as depicted in column 8 of Table II, application no. 3, preferably a homolog or functional equivalent as depicted in column 8 of Table II B, application no. 3, and being depicted in the same respective line as said B0348, and preferably the activity is increased plastidic.

Accordingly, in one embodiment, the molecule which activity is to be increased in the process of the invention is the gene product with an activity as a “2,3-dihydroxyphenylpropionate 1,2-dioxygenase”, preferably it is the molecule of section (a) or (b) of this paragraph.

In particular, it was observed that in plants, especially in Arabidopsis thaliana, increasing or generating the activity of a gene product plastidic with the activity of a “2,3-dihydroxyphenylpropionate 1,2-dioxygenase”, preferably being encoded by a gene comprising the nucleic acid sequence SEQ ID NO. 7269, preferably the coding region thereof, conferred the production of or the increase in serine compared with the wild type control.

The nucleic acid sequence of B0348 from Escherichia coli, e.g. as shown in the respective line in column 5 of Table I, application no. 3, has been published in B. And the activity of the gene product thereof is the activity of 2,3-dihydroxyphenylpropionate 1,2-dioxygenase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “2,3-dihydroxyphenylpropionate 1,2-dioxygenase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0348, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0348, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0348, or a functional equivalent or a homolog thereof as depicted in column 8 of Table II, application no. 3, preferably a homolog or functional equivalent as depicted in column 8 of Table II B, application no. 3, and being depicted in the same respective line as said B0348, and preferably the activity is increased plastidic.

Accordingly, in one embodiment, the molecule which activity is to be increased in the process of the invention is the gene product with an activity as a “2,3-dihydroxyphenylpropionate 1,2-dioxygenase”, preferably it is the molecule of section (a) or (b) of this paragraph.

In particular, it was observed that in plants, especially in Arabidopsis thaliana, increasing or generating the activity of a gene product plastidic with the activity of a “2,3-dihydroxyphenylpropionate 1,2-dioxygenase”, preferably being encoded by a gene comprising the nucleic acid sequence SEQ ID NO. 7269, preferably the coding region thereof, conferred the production of or the increase in tyrosine compared with the wild type control.

The nucleic acid sequence of B0391 from Escherichia coli, e.g. as shown in the respective line in column 5 of Table I, application no. 3, has been published in B. And the activity of the gene product thereof is the activity of b0391-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “b0391-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0391, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0391, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0391, or a functional equivalent or a homolog thereof as depicted in column 8 of Table II, application no. 3, preferably a homolog or functional equivalent as depicted in column 8 of Table II B, application no. 3, and being depicted in the same respective line as said B0391, and preferably the activity is increased plastidic.

Accordingly, in one embodiment, the molecule which activity is to be increased in the process of the invention is the gene product with an activity as a “b0391-protein”, preferably it is the molecule of section (a) or (b) of this paragraph.

In particular, it was observed that in plants, especially in Arabidopsis thaliana, increasing or generating the activity of a gene product plastidic with the activity of a “b0391-protein”, preferably being encoded by a gene comprising the nucleic acid sequence SEQ ID NO. 75195, preferably the coding region thereof, conferred the production of or the increase in aspartate compared with the wild type control.

The nucleic acid sequence of B0449 from Escherichia coli, e.g. as shown in the respective line in column 5 of Table I, application no. 3, has been published in B. And the activity of the gene product thereof is the activity of ATP-binding component of a transport system.

Accordingly, in one embodiment, the process of the present invention for producing asparagine in a non-human organism, like a microorganism or a plant or a part thereof, comprises increasing or generating the activity of a gene product with the activity of a gene product conferring the activity of “ATP-binding component of a transport system”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0449, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0449, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0449, or a functional equivalent or a homolog thereof as depicted in column 8 of Table II, application no. 3, preferably a homolog or functional equivalent as depicted in column 8 of Table II B, application no. 3, and being depicted in the same respective line as said B0449, and preferably the activity is increased non-targeted.

Accordingly, in one embodiment, the molecule which activity is to be increased in the process of the invention is the gene product with an activity as a “ATP-binding component of a transport system”, preferably it is the molecule of section (a) or (b) of this paragraph.

In particular, it was observed that in plants, especially in Arabidopsis thaliana, increasing or generating the activity of a gene product non-targeted with the activity of a “ATP-binding component of a transport system”, preferably being encoded by a gene comprising the nucleic acid sequence SEQ ID NO. 7333, preferably the coding region thereof, conferred the production of or the increase in asparagine compared with the wild type control.

The nucleic acid sequence of B0449 from Escherichia coli, e.g. as shown in the respective line in column 5 of Table I, application no. 3, has been published in B. And the activity of the gene product thereof is the activity of ATP-binding component of a transport system.

Accordingly, in one embodiment, the process of the present invention for producing aspartate in a non-human organism, like a microorganism or a plant or a part thereof, comprises increasing or generating the activity of a gene product with the activity of a gene product conferring the activity of “ATP-binding component of a transport system”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0449, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0449, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0449, or a functional equivalent or a homolog thereof as depicted in column 8 of Table II, application no. 3, preferably a homolog or functional equivalent as depicted in column 8 of Table II B, application no. 3, and being depicted in the same respective line as said B0449, and preferably the activity is increased non-targeted.

Accordingly, in one embodiment, the molecule which activity is to be increased in the process of the invention is the gene product with an activity as a “ATP-binding component of a transport system”, preferably it is the molecule of section (a) or (b) of this paragraph.

In particular, it was observed that in plants, especially in Arabidopsis thaliana, increasing or generating the activity of a gene product non-targeted with the activity of a “ATP-binding component of a transport system”, preferably being encoded by a gene comprising the nucleic acid sequence SEQ ID NO. 7333, preferably the coding region thereof, conferred the production of or the increase in aspartate compared with the wild type control.

The nucleic acid sequence of B0449 from Escherichia coli, e.g. as shown in the respective line in column 5 of Table I, application no. 3, has been published in B. And the activity of the gene product thereof is the activity of ATP-binding component of a transport system.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “ATP-binding component of a transport system”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0449, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0449, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0449, or a functional equivalent or a homolog thereof as depicted in column 8 of Table II, application no. 3, preferably a homolog or functional equivalent as depicted in column 8 of Table II B, application no. 3, and being depicted in the same respective line as said B0449, and preferably the activity is increased non-targeted.

Accordingly, in one embodiment, the molecule which activity is to be increased in the process of the invention is the gene product with an activity as a “ATP-binding component of a transport system”, preferably it is the molecule of section (a) or (b) of this paragraph.

In particular, it was observed that in plants, especially in Arabidopsis thaliana, increasing or generating the activity of a gene product non-targeted with the activity of a “ATP-binding component of a transport system”, preferably being encoded by a gene comprising the nucleic acid sequence SEQ ID NO. 7333, preferably the coding region thereof, conferred the production of or the increase in phenylalanine compared with the wild type control.

The nucleic acid sequence of B0449 from Escherichia coli, e.g. as shown in the respective line in column 5 of Table I, application no. 3, has been published in B. And the activity of the gene product thereof is the activity of ATP-binding component of a transport system.

Accordingly, in one embodiment, the process of the present invention for producing serine in a non-human organism, like a microorganism or a plant or a part thereof, comprises increasing or generating the activity of a gene product with the activity of a gene product conferring the activity of “ATP-binding component of a transport system”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0449, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0449, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0449, or a functional equivalent or a homolog thereof as depicted in column 8 of Table II, application no. 3, preferably a homolog or functional equivalent as depicted in column 8 of Table II B, application no. 3, and being depicted in the same respective line as said B0449, and preferably the activity is increased non-targeted.

Accordingly, in one embodiment, the molecule which activity is to be increased in the process of the invention is the gene product with an activity as a “ATP-binding component of a transport system”, preferably it is the molecule of section (a) or (b) of this paragraph.

In particular, it was observed that in plants, especially in Arabidopsis thaliana, increasing or generating the activity of a gene product non-targeted with the activity of a “ATP-binding component of a transport system”, preferably being encoded by a gene comprising the nucleic acid sequence SEQ ID NO. 7333, preferably the coding region thereof, conferred the production of or the increase in serine compared with the wild type control.

The nucleic acid sequence of B0449 from Escherichia coli, e.g. as shown in the respective line in column 5 of Table I, application no. 3, has been published in B. And the activity of the gene product thereof is the activity of ATP-binding component of a transport system.

Accordingly, in one embodiment, the process of the present invention for producing tyrosine in a non-human organism, like a microorganism or a plant or a part thereof, comprises increasing or generating the activity of a gene product with the activity of a gene product conferring the activity of “ATP-binding component of a transport system”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0449, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0449, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0449, or a functional equivalent or a homolog thereof as depicted in column 8 of Table II, application no. 3, preferably a homolog or functional equivalent as depicted in column 8 of Table II B, application no. 3, and being depicted in the same respective line as said B0449, and preferably the activity is increased non-targeted.

Accordingly, in one embodiment, the molecule which activity is to be increased in the process of the invention is the gene product with an activity as a “ATP-binding component of a transport system”, preferably it is the molecule of section (a) or (b) of this paragraph.

In particular, it was observed that in plants, especially in Arabidopsis thaliana, increasing or generating the activity of a gene product non-targeted with the activity of a “ATP-binding component of a transport system”, preferably being encoded by a gene comprising the nucleic acid sequence SEQ ID NO. 7333, preferably the coding region thereof, conferred the production of or the increase in tyrosine compared with the wild type control.

The nucleic acid sequence of B0456 from Escherichia coli, e.g. as shown in the respective line in column 5 of Table I, application no. 3, has been published in B. And the activity of the gene product thereof is the activity of b0456-protein.

Accordingly, in one embodiment, the process of the present invention for producing alanine in a non-human organism, like a microorganism or a plant or a part thereof, comprises increasing or generating the activity of a gene product with the activity of a gene product conferring the activity of “b0456-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0456, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0456, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0456, or a functional equivalent or a homolog thereof as depicted in column 8 of Table II, application no. 3, preferably a homolog or functional equivalent as depicted in column 8 of Table II B, application no. 3, and being depicted in the same respective line as said B0456, and preferably the activity is increased non-targeted.

Accordingly, in one embodiment, the molecule which activity is to be increased in the process of the invention is the gene product with an activity as a “b0456-protein”, preferably it is the molecule of section (a) or (b) of this paragraph.

In particular, it was observed that in plants, especially in Arabidopsis thaliana, increasing or generating the activity of a gene product non-targeted with the activity of a “b0456-protein”, preferably being encoded by a gene comprising the nucleic acid sequence SEQ ID NO. 35875, preferably the coding region thereof, conferred the production of or the increase in alanine compared with the wild type control.

The nucleic acid sequence of B0486 from Escherichia coli, e.g. as shown in the respective line in column 5 of Table I, application no. 3, has been published in B. And the activity of the gene product thereof is the activity of membrane transport protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “membrane transport protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0486, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0486, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0486, or a functional equivalent or a homolog thereof as depicted in column 8 of Table II, application no. 3, preferably a homolog or functional equivalent as depicted in column 8 of Table II B, application no. 3, and being depicted in the same respective line as said B0486, and preferably the activity is increased non-targeted.

Accordingly, in one embodiment, the molecule which activity is to be increased in the process of the invention is the gene product with an activity as a “membrane transport protein”, preferably it is the molecule of section (a) or (b) of this paragraph.

In particular, it was observed that in plants, especially in Arabidopsis thaliana, increasing or generating the activity of a gene product non-targeted with the activity of a “membrane transport protein”, preferably being encoded by a gene comprising the nucleic acid sequence SEQ ID NO. 7686, preferably the coding region thereof, conferred the production of or the increase in aspartate compared with the wild type control.

The nucleic acid sequence of B0486 from Escherichia coli, e.g. as shown in the respective line in column 5 of Table I, application no. 3, has been published in B. And the activity of the gene product thereof is the activity of membrane transport protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “membrane transport protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0486, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0486, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0486, or a functional equivalent or a homolog thereof as depicted in column 8 of Table II, application no. 3, preferably a homolog or functional equivalent as depicted in column 8 of Table II B, application no. 3, and being depicted in the same respective line as said B0486, and preferably the activity is increased non-targeted.

Accordingly, in one embodiment, the molecule which activity is to be increased in the process of the invention is the gene product with an activity as a “membrane transport protein”, preferably it is the molecule of section (a) or (b) of this paragraph.

In particular, it was observed that in plants, especially in Arabidopsis thaliana, increasing or generating the activity of a gene product non-targeted with the activity of a “membrane transport protein”, preferably being encoded by a gene comprising the nucleic acid sequence SEQ ID NO. 7686, preferably the coding region thereof, conferred the production of or the increase in phenylalanine compared with the wild type control.

The nucleic acid sequence of B0486 from Escherichia coli, e.g. as shown in the respective line in column 5 of Table I, application no. 3, has been published in B. And the activity of the gene product thereof is the activity of membrane transport protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “membrane transport protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0486, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0486, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0486, or a functional equivalent or a homolog thereof as depicted in column 8 of Table II, application no. 3, preferably a homolog or functional equivalent as depicted in column 8 of Table II B, application no. 3, and being depicted in the same respective line as said B0486, and preferably the activity is increased non-targeted.

Accordingly, in one embodiment, the molecule which activity is to be increased in the process of the invention is the gene product with an activity as a “membrane transport protein”, preferably it is the molecule of section (a) or (b) of this paragraph.

In particular, it was observed that in plants, especially in Arabidopsis thaliana, increasing or generating the activity of a gene product non-targeted with the activity of a “membrane transport protein”, preferably being encoded by a gene comprising the nucleic acid sequence SEQ ID NO. 7686, preferably the coding region thereof, conferred the production of or the increase in tyrosine compared with the wild type control.

The nucleic acid sequence of B0518 from Escherichia coli, e.g. as shown in the respective line in column 5 of Table I, application no. 3, has been published in B. And the activity of the gene product thereof is the activity of b0518-protein.

Accordingly, in one embodiment, the process of the present invention for producing homoserine in a non-human organism, like a microorganism or a plant or a part thereof, comprises increasing or generating the activity of a gene product with the activity of a gene product conferring the activity of “b0518-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0518, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0518, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0518, or a functional equivalent or a homolog thereof as depicted in column 8 of Table II, application no. 3, preferably a homolog or functional equivalent as depicted in column 8 of Table II B, application no. 3, and being depicted in the same respective line as said B0518, and preferably the activity is increased non-targeted.

Accordingly, in one embodiment, the molecule which activity is to be increased in the process of the invention is the gene product with an activity as a “b0518-protein”, preferably it is the molecule of section (a) or (b) of this paragraph.

In particular, it was observed that in plants, especially in Arabidopsis thaliana, increasing or generating the activity of a gene product non-targeted with the activity of a “b0518-protein”, preferably being encoded by a gene comprising the nucleic acid sequence SEQ ID NO. 35936, preferably the coding region thereof, conferred the production of or the increase in homoserine compared with the wild type control.

The nucleic acid sequence of B0593 from Escherichia coli, e.g. as shown in the respective line in column 5 of Table I, application no. 3, has been published in B. And the activity of the gene product thereof is the activity of isochorismate synthase.

Accordingly, in one embodiment, the process of the present invention for producing aspartate in a non-human organism, like a microorganism or a plant or a part thereof, comprises increasing or generating the activity of a gene product with the activity of a gene product conferring the activity of “isochorismate synthase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0593, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0593, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0593, or a functional equivalent or a homolog thereof as depicted in column 8 of Table II, application no. 3, preferably a homolog or functional equivalent as depicted in column 8 of Table II B, application no. 3, and being depicted in the same respective line as said B0593, and preferably the activity is increased plastidic.

Accordingly, in one embodiment, the molecule which activity is to be increased in the process of the invention is the gene product with an activity as a “isochorismate synthase”, preferably it is the molecule of section (a) or (b) of this paragraph.

In particular, it was observed that in plants, especially in Arabidopsis thaliana, increasing or generating the activity of a gene product plastidic with the activity of a “isochorismate synthase”, preferably being encoded by a gene comprising the nucleic acid sequence SEQ ID NO. 35967, preferably the coding region thereof, conferred the production of or the increase in aspartate compared with the wild type control.

The nucleic acid sequence of B0593 from Escherichia coli, e.g. as shown in the respective line in column 5 of Table I, application no. 3, has been published in B. And the activity of the gene product thereof is the activity of isochorismate synthase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “isochorismate synthase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0593, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0593, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0593, or a functional equivalent or a homolog thereof as depicted in column 8 of Table II, application no. 3, preferably a homolog or functional equivalent as depicted in column 8 of Table II B, application no. 3, and being depicted in the same respective line as said B0593, and preferably the activity is increased plastidic.

Accordingly, in one embodiment, the molecule which activity is to be increased in the process of the invention is the gene product with an activity as a “isochorismate synthase”, preferably it is the molecule of section (a) or (b) of this paragraph.

In particular, it was observed that in plants, especially in Arabidopsis thaliana, increasing or generating the activity of a gene product plastidic with the activity of a “isochorismate synthase”, preferably being encoded by a gene comprising the nucleic acid sequence SEQ ID NO. 35967, preferably the coding region thereof, conferred the production of or the increase in phenylalanine compared with the wild type control.

The nucleic acid sequence of B0593 from Escherichia coli, e.g. as shown in the respective line in column 5 of Table I, application no. 3, has been published in B. And the activity of the gene product thereof is the activity of isochorismate synthase.

Accordingly, in one embodiment, the process of the present invention for producing serine in a non-human organism, like a microorganism or a plant or a part thereof, comprises increasing or generating the activity of a gene product with the activity of a gene product conferring the activity of “isochorismate synthase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0593, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0593, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0593, or a functional equivalent or a homolog thereof as depicted in column 8 of Table II, application no. 3, preferably a homolog or functional equivalent as depicted in column 8 of Table II B, application no. 3, and being depicted in the same respective line as said B0593, and preferably the activity is increased plastidic.

Accordingly, in one embodiment, the molecule which activity is to be increased in the process of the invention is the gene product with an activity as a “isochorismate synthase”, preferably it is the molecule of section (a) or (b) of this paragraph.

In particular, it was observed that in plants, especially in Arabidopsis thaliana, increasing or generating the activity of a gene product plastidic with the activity of a “isochorismate synthase”, preferably being encoded by a gene comprising the nucleic acid sequence SEQ ID NO. 35967, preferably the coding region thereof, conferred the production of or the increase in serine compared with the wild type control.

The nucleic acid sequence of B0593 from Escherichia coli, e.g. as shown in the respective line in column 5 of Table I, application no. 3, has been published in B. And the activity of the gene product thereof is the activity of isochorismate synthase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “isochorismate synthase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0593, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0593, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0593, or a functional equivalent or a homolog thereof as depicted in column 8 of Table II, application no. 3, preferably a homolog or functional equivalent as depicted in column 8 of Table II B, application no. 3, and being depicted in the same respective line as said B0593, and preferably the activity is increased plastidic.

Accordingly, in one embodiment, the molecule which activity is to be increased in the process of the invention is the gene product with an activity as a “isochorismate synthase”, preferably it is the molecule of section (a) or (b) of this paragraph.

In particular, it was observed that in plants, especially in Arabidopsis thaliana, increasing or generating the activity of a gene product plastidic with the activity of a “isochorismate synthase”, preferably being encoded by a gene comprising the nucleic acid sequence SEQ ID NO. 35967, preferably the coding region thereof, conferred the production of or the increase in tyrosine compared with the wild type control.

The nucleic acid sequence of B0593 from Escherichia coli, e.g. as shown in the respective line in column 5 of Table I, application no. 3, has been published in B. And the activity of the gene product thereof is the activity of isochorismate synthase.

Accordingly, in one embodiment, the process of the present invention for producing 3,4-dihydroxyphenylalanine (dopa) in a non-human organism, like a microorganism or a plant or a part thereof, comprises increasing or generating the activity of a gene product with the activity of a gene product conferring the activity of “isochorismate synthase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0593, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0593, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0593, or a functional equivalent or a homolog thereof as depicted in column 8 of Table II, application no. 3, preferably a homolog or functional equivalent as depicted in column 8 of Table II B, application no. 3, and being depicted in the same respective line as said B0593, and preferably the activity is increased plastidic.

Accordingly, in one embodiment, the molecule which activity is to be increased in the process of the invention is the gene product with an activity as a “isochorismate synthase”, preferably it is the molecule of section (a) or (b) of this paragraph.

In particular, it was observed that in plants, especially in Arabidopsis thaliana, increasing or generating the activity of a gene product plastidic with the activity of a “isochorismate synthase”, preferably being encoded by a gene comprising the nucleic acid sequence SEQ ID NO. 35967, preferably the coding region thereof, conferred the production of or the increase in 3,4-dihydroxyphenylalanine (dopa) compared with the wild type control.

The nucleic acid sequence of B0593 from Escherichia coli, e.g. as shown in the respective line in column 5 of Table I, application no. 3, has been published in B. And the activity of the gene product thereof is the activity of isochorismate synthase.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “isochorismate synthase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0593, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0593, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0593, or a functional equivalent or a homolog thereof as depicted in column 8 of Table II, application no. 3, preferably a homolog or functional equivalent as depicted in column 8 of Table II B, application no. 3, and being depicted in the same respective line as said B0593, and preferably the activity is increased plastidic.

Accordingly, in one embodiment, the molecule which activity is to be increased in the process of the invention is the gene product with an activity as a “isochorismate synthase”, preferably it is the molecule of section (a) or (b) of this paragraph.

In particular, it was observed that in plants, especially in Arabidopsis thaliana, increasing or generating the activity of a gene product plastidic with the activity of a “isochorismate synthase”, preferably being encoded by a gene comprising the nucleic acid sequence SEQ ID NO. 35967, preferably the coding region thereof, conferred the production of or the increase in 5-oxoproline compared with the wild type control.

The nucleic acid sequence of B0752 from Escherichia coli, e.g. as shown in the respective line in column 5 of Table I, application no. 3, has been published in B. And the activity of the gene product thereof is the activity of zinc transporter.

Accordingly, in one embodiment, the process of the present invention for producing tyrosine in a non-human organism, like a microorganism or a plant or a part thereof, comprises increasing or generating the activity of a gene product with the activity of a gene product conferring the activity of “zinc transporter”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0752, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0752, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0752, or a functional equivalent or a homolog thereof as depicted in column 8 of Table II, application no. 3, preferably a homolog or functional equivalent as depicted in column 8 of Table II B, application no. 3, and being depicted in the same respective line as said B0752, and preferably the activity is increased non-targeted.

Accordingly, in one embodiment, the molecule which activity is to be increased in the process of the invention is the gene product with an activity as a “zinc transporter”, preferably it is the molecule of section (a) or (b) of this paragraph.

In particular, it was observed that in plants, especially in Arabidopsis thaliana, increasing or generating the activity of a gene product non-targeted with the activity of a “zinc transporter”, preferably being encoded by a gene comprising the nucleic acid sequence SEQ ID NO. 36114, preferably the coding region thereof, conferred the production of or the increase in tyrosine compared with the wild type control.

The nucleic acid sequence of B0754 from Escherichia coli, e.g. as shown in the respective line in column 5 of Table I, application no. 3, has been published in B. And the activity of the gene product thereof is the activity of 3-deoxy-D-arabinoheptulosonate-7-phosphatesynthase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “3-deoxy-D-arabinoheptulosonate-7-phosphatesynthase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0754, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0754, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0754, or a functional equivalent or a homolog thereof as depicted in column 8 of Table II, application no. 3, preferably a homolog or functional equivalent as depicted in column 8 of Table II B, application no. 3, and being depicted in the same respective line as said B0754, and preferably the activity is increased plastidic.

Accordingly, in one embodiment, the molecule which activity is to be increased in the process of the invention is the gene product with an activity as a “3-deoxy-D-arabinoheptulosonate-7-phosphatesynthase”, preferably it is the molecule of section (a) or (b) of this paragraph.

In particular, it was observed that in plants, especially in Arabidopsis thaliana, increasing or generating the activity of a gene product plastidic with the activity of a “3-deoxy-D-arabinoheptulosonate-7-phosphatesynthase”, preferably being encoded by a gene comprising the nucleic acid sequence SEQ ID NO. 75286, preferably the coding region thereof, conferred the production of or the increase in tyrosine compared with the wild type control.

The nucleic acid sequence of B0801 from Escherichia coli, e.g. as shown in the respective line in column 5 of Table I, application no. 3, has been published in B. And the activity of the gene product thereof is the activity of b0801-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “b0801-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0801, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0801, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0801, or a functional equivalent or a homolog thereof as depicted in column 8 of Table II, application no. 3, preferably a homolog or functional equivalent as depicted in column 8 of Table II B, application no. 3, and being depicted in the same respective line as said B0801, and preferably the activity is increased non-targeted.

Accordingly, in one embodiment, the molecule which activity is to be increased in the process of the invention is the gene product with an activity as a “b0801-protein”, preferably it is the molecule of section (a) or (b) of this paragraph.

In particular, it was observed that in plants, especially in Arabidopsis thaliana, increasing or generating the activity of a gene product non-targeted with the activity of a “b0801-protein”, preferably being encoded by a gene comprising the nucleic acid sequence SEQ ID NO. 75807, preferably the coding region thereof, conferred the production of or the increase in tyrosine compared with the wild type control.

The nucleic acid sequence of B0828 from Escherichia coli, e.g. as shown in the respective line in column 5 of Table I, application no. 3, has been published in B. And the activity of the gene product thereof is the activity of asparaginase.

Accordingly, in one embodiment, the process of the present invention for producing alanine in a non-human organism, like a microorganism or a plant or a part thereof, comprises increasing or generating the activity of a gene product with the activity of a gene product conferring the activity of “asparaginase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0828, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0828, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0828, or a functional equivalent or a homolog thereof as depicted in column 8 of Table II, application no. 3, preferably a homolog or functional equivalent as depicted in column 8 of Table II B, application no. 3, and being depicted in the same respective line as said B0828, and preferably the activity is increased non-targeted.

Accordingly, in one embodiment, the molecule which activity is to be increased in the process of the invention is the gene product with an activity as a “asparaginase”, preferably it is the molecule of section (a) or (b) of this paragraph.

In particular, it was observed that in plants, especially in Arabidopsis thaliana, increasing or generating the activity of a gene product non-targeted with the activity of a “asparaginase”, preferably being encoded by a gene comprising the nucleic acid sequence SEQ ID NO. 36299, preferably the coding region thereof, conferred the production of or the increase in alanine compared with the wild type control.

The nucleic acid sequence of B0841 from Escherichia coli, e.g. as shown in the respective line in column 5 of Table I, application no. 3, has been published in B. And the activity of the gene product thereof is the activity of b0841-protein.

Accordingly, in one embodiment, the process of the present invention for producing glycine in a non-human organism, like a microorganism or a plant or a part thereof, comprises increasing or generating the activity of a gene product with the activity of a gene product conferring the activity of “b0841-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0841, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0841, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0841, or a functional equivalent or a homolog thereof as depicted in column 8 of Table II, application no. 3, preferably a homolog or functional equivalent as depicted in column 8 of Table II B, application no. 3, and being depicted in the same respective line as said B0841, and preferably the activity is increased non-targeted.

Accordingly, in one embodiment, the molecule which activity is to be increased in the process of the invention is the gene product with an activity as a “b0841-protein”, preferably it is the molecule of section (a) or (b) of this paragraph.

In particular, it was observed that in plants, especially in Arabidopsis thaliana, increasing or generating the activity of a gene product non-targeted with the activity of a “b0841-protein”, preferably being encoded by a gene comprising the nucleic acid sequence SEQ ID NO. 75873, preferably the coding region thereof, conferred the production of or the increase in glycine compared with the wild type control.

The nucleic acid sequence of B0841 from Escherichia coli, e.g. as shown in the respective line in column 5 of Table I, application no. 3, has been published in B. And the activity of the gene product thereof is the activity of b0841-protein.

Accordingly, in one embodiment, the process of the present invention for producing homoserine in a non-human organism, like a microorganism or a plant or a part thereof, comprises increasing or generating the activity of a gene product with the activity of a gene product conferring the activity of “b0841-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0841, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0841, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0841, or a functional equivalent or a homolog thereof as depicted in column 8 of Table II, application no. 3, preferably a homolog or functional equivalent as depicted in column 8 of Table II B, application no. 3, and being depicted in the same respective line as said B0841, and preferably the activity is increased non-targeted.

Accordingly, in one embodiment, the molecule which activity is to be increased in the process of the invention is the gene product with an activity as a “b0841-protein”, preferably it is the molecule of section (a) or (b) of this paragraph.

In particular, it was observed that in plants, especially in Arabidopsis thaliana, increasing or generating the activity of a gene product non-targeted with the activity of a “b0841-protein”, preferably being encoded by a gene comprising the nucleic acid sequence SEQ ID NO. 75873, preferably the coding region thereof, conferred the production of or the increase in homoserine compared with the wild type control.

The nucleic acid sequence of B0898 from Escherichia coli, e.g. as shown in the respective line in column 5 of Table I, application no. 3, has been published in B. And the activity of the gene product thereof is the activity of major facilitator superfamily transporter protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “major facilitator superfamily transporter protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0898, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0898, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0898, or a functional equivalent or a homolog thereof as depicted in column 8 of Table II, application no. 3, preferably a homolog or functional equivalent as depicted in column 8 of Table II B, application no. 3, and being depicted in the same respective line as said B0898, and preferably the activity is increased non-targeted.

Accordingly, in one embodiment, the molecule which activity is to be increased in the process of the invention is the gene product with an activity as a “major facilitator superfamily transporter protein”, preferably it is the molecule of section (a) or (b) of this paragraph.

In particular, it was observed that in plants, especially in Arabidopsis thaliana, increasing or generating the activity of a gene product non-targeted with the activity of a “major facilitator superfamily transporter protein”, preferably being encoded by a gene comprising the nucleic acid sequence SEQ ID NO. 7917, preferably the coding region thereof, conferred the production of or the increase in phenylalanine compared with the wild type control.

The nucleic acid sequence of B0898 from Escherichia coli, e.g. as shown in the respective line in column 5 of Table I, application no. 3, has been published in B. And the activity of the gene product thereof is the activity of major facilitator superfamily transporter protein.

Accordingly, in one embodiment, the process of the present invention for producing serine in a non-human organism, like a microorganism or a plant or a part thereof, comprises increasing or generating the activity of a gene product with the activity of a gene product conferring the activity of “major facilitator superfamily transporter protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0898, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0898, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0898, or a functional equivalent or a homolog thereof as depicted in column 8 of Table II, application no. 3, preferably a homolog or functional equivalent as depicted in column 8 of Table II B, application no. 3, and being depicted in the same respective line as said B0898, and preferably the activity is increased non-targeted.

Accordingly, in one embodiment, the molecule which activity is to be increased in the process of the invention is the gene product with an activity as a “major facilitator superfamily transporter protein”, preferably it is the molecule of section (a) or (b) of this paragraph.

In particular, it was observed that in plants, especially in Arabidopsis thaliana, increasing or generating the activity of a gene product non-targeted with the activity of a “major facilitator superfamily transporter protein”, preferably being encoded by a gene comprising the nucleic acid sequence SEQ ID NO. 7917, preferably the coding region thereof, conferred the production of or the increase in serine compared with the wild type control.

The nucleic acid sequence of B0898 from Escherichia coli, e.g. as shown in the respective line in column 5 of Table I, application no. 3, has been published in B. And the activity of the gene product thereof is the activity of major facilitator superfamily transporter protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “major facilitator superfamily transporter protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0898, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0898, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0898, or a functional equivalent or a homolog thereof as depicted in column 8 of Table II, application no. 3, preferably a homolog or functional equivalent as depicted in column 8 of Table II B, application no. 3, and being depicted in the same respective line as said B0898, and preferably the activity is increased non-targeted.

Accordingly, in one embodiment, the molecule which activity is to be increased in the process of the invention is the gene product with an activity as a “major facilitator superfamily transporter protein”, preferably it is the molecule of section (a) or (b) of this paragraph.

In particular, it was observed that in plants, especially in Arabidopsis thaliana, increasing or generating the activity of a gene product non-targeted with the activity of a “major facilitator superfamily transporter protein”, preferably being encoded by a gene comprising the nucleic acid sequence SEQ ID NO. 7917, preferably the coding region thereof, conferred the production of or the increase in tyrosine compared with the wild type control.

The nucleic acid sequence of B0898 from Escherichia coli, e.g. as shown in the respective line in column 5 of Table I, application no. 3, has been published in B. And the activity of the gene product thereof is the activity of major facilitator superfamily transporter protein.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “major facilitator superfamily transporter protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0898, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0898, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0898, or a functional equivalent or a homolog thereof as depicted in column 8 of Table II, application no. 3, preferably a homolog or functional equivalent as depicted in column 8 of Table II B, application no. 3, and being depicted in the same respective line as said B0898, and preferably the activity is increased non-targeted.

Accordingly, in one embodiment, the molecule which activity is to be increased in the process of the invention is the gene product with an activity as a “major facilitator superfamily transporter protein”, preferably it is the molecule of section (a) or (b) of this paragraph.

In particular, it was observed that in plants, especially in Arabidopsis thaliana, increasing or generating the activity of a gene product non-targeted with the activity of a “major facilitator superfamily transporter protein”, preferably being encoded by a gene comprising the nucleic acid sequence SEQ ID NO. 7917, preferably the coding region thereof, conferred the production of or the increase in 5-oxoproline compared with the wild type control.

The nucleic acid sequence of B0917 from Escherichia coli, e.g. as shown in the respective line in column 5 of Table I, application no. 3, has been published in B. And the activity of the gene product thereof is the activity of b0917-protein.

Accordingly, in one embodiment, the process of the present invention for producing citrulline in a non-human organism, like a microorganism or a plant or a part thereof, comprises increasing or generating the activity of a gene product with the activity of a gene product conferring the activity of “b0917-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0917, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0917, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0917, or a functional equivalent or a homolog thereof as depicted in column 8 of Table II, application no. 3, preferably a homolog or functional equivalent as depicted in column 8 of Table II B, application no. 3, and being depicted in the same respective line as said B0917, and preferably the activity is increased non-targeted.

Accordingly, in one embodiment, the molecule which activity is to be increased in the process of the invention is the gene product with an activity as a “b0917-protein”, preferably it is the molecule of section (a) or (b) of this paragraph.

In particular, it was observed that in plants, especially in Arabidopsis thaliana, increasing or generating the activity of a gene product non-targeted with the activity of a “b0917-protein”, preferably being encoded by a gene comprising the nucleic acid sequence SEQ ID NO. 75915, preferably the coding region thereof, conferred the production of or the increase in citrulline compared with the wild type control.

The nucleic acid sequence of B0917 from Escherichia coli, e.g. as shown in the respective line in column 5 of Table I, application no. 3, has been published in B. And the activity of the gene product thereof is the activity of b0917-protein.

Accordingly, in one embodiment, the process of the present invention for producing glycine in a non-human organism, like a microorganism or a plant or a part thereof, comprises increasing or generating the activity of a gene product with the activity of a gene product conferring the activity of “b0917-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0917, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0917, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0917, or a functional equivalent or a homolog thereof as depicted in column 8 of Table II, application no. 3, preferably a homolog or functional equivalent as depicted in column 8 of Table II B, application no. 3, and being depicted in the same respective line as said B0917, and preferably the activity is increased non-targeted.

Accordingly, in one embodiment, the molecule which activity is to be increased in the process of the invention is the gene product with an activity as a “b0917-protein”, preferably it is the molecule of section (a) or (b) of this paragraph.

In particular, it was observed that in plants, especially in Arabidopsis thaliana, increasing or generating the activity of a gene product non-targeted with the activity of a “b0917-protein”, preferably being encoded by a gene comprising the nucleic acid sequence SEQ ID NO. 75915, preferably the coding region thereof, conferred the production of or the increase in glycine compared with the wild type control.

The nucleic acid sequence of B0917 from Escherichia coli, e.g. as shown in the respective line in column 5 of Table I, application no. 3, has been published in B. And the activity of the gene product thereof is the activity of b0917-protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “b0917-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0917, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0917, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0917, or a functional equivalent or a homolog thereof as depicted in column 8 of Table II, application no. 3, preferably a homolog or functional equivalent as depicted in column 8 of Table II B, application no. 3, and being depicted in the same respective line as said B0917, and preferably the activity is increased non-targeted.

Accordingly, in one embodiment, the molecule which activity is to be increased in the process of the invention is the gene product with an activity as a “b0917-protein”, preferably it is the molecule of section (a) or (b) of this paragraph.

In particular, it was observed that in plants, especially in Arabidopsis thaliana, increasing or generating the activity of a gene product non-targeted with the activity of a “b0917-protein”, preferably being encoded by a gene comprising the nucleic acid sequence SEQ ID NO. 75915, preferably the coding region thereof, conferred the production of or the increase in phenylalanine compared with the wild type control.

The nucleic acid sequence of B0917 from Escherichia coli, e.g. as shown in the respective line in column 5 of Table I, application no. 3, has been published in B. And the activity of the gene product thereof is the activity of b0917-protein.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “b0917-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0917, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0917, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0917, or a functional equivalent or a homolog thereof as depicted in column 8 of Table II, application no. 3, preferably a homolog or functional equivalent as depicted in column 8 of Table II B, application no. 3, and being depicted in the same respective line as said B0917, and preferably the activity is increased non-targeted.

Accordingly, in one embodiment, the molecule which activity is to be increased in the process of the invention is the gene product with an activity as a “b0917-protein”, preferably it is the molecule of section (a) or (b) of this paragraph.

In particular, it was observed that in plants, especially in Arabidopsis thaliana, increasing or generating the activity of a gene product non-targeted with the activity of a “b0917-protein”, preferably being encoded by a gene comprising the nucleic acid sequence SEQ ID NO. 75915, preferably the coding region thereof, conferred the production of or the increase in 5-oxoproline compared with the wild type control.

The nucleic acid sequence of B0963 from Escherichia coli, e.g. as shown in the respective line in column 5 of Table I, application no. 3, has been published in B. And the activity of the gene product thereof is the activity of methylglyoxal synthase.

Accordingly, in one embodiment, the process of the present invention for producing alanine in a non-human organism, like a microorganism or a plant or a part thereof, comprises increasing or generating the activity of a gene product with the activity of a gene product conferring the activity of “methylglyoxal synthase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0963, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0963, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0963, or a functional equivalent or a homolog thereof as depicted in column 8 of Table II, application no. 3, preferably a homolog or functional equivalent as depicted in column 8 of Table II B, application no. 3, and being depicted in the same respective line as said B0963, and preferably the activity is increased non-targeted.

Accordingly, in one embodiment, the molecule which activity is to be increased in the process of the invention is the gene product with an activity as a “methylglyoxal synthase”, preferably it is the molecule of section (a) or (b) of this paragraph.

In particular, it was observed that in plants, especially in Arabidopsis thaliana, increasing or generating the activity of a gene product non-targeted with the activity of a “methylglyoxal synthase”, preferably being encoded by a gene comprising the nucleic acid sequence SEQ ID NO. 36670, preferably the coding region thereof, conferred the production of or the increase in alanine compared with the wild type control.

The nucleic acid sequence of B0963 from Escherichia coli, e.g. as shown in the respective line in column 5 of Table I, application no. 3, has been published in B. And the activity of the gene product thereof is the activity of methylglyoxal synthase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “methylglyoxal synthase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0963, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0963, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0963, or a functional equivalent or a homolog thereof as depicted in column 8 of Table II, application no. 3, preferably a homolog or functional equivalent as depicted in column 8 of Table II B, application no. 3, and being depicted in the same respective line as said B0963, and preferably the activity is increased non-targeted.

Accordingly, in one embodiment, the molecule which activity is to be increased in the process of the invention is the gene product with an activity as a “methylglyoxal synthase”, preferably it is the molecule of section (a) or (b) of this paragraph.

In particular, it was observed that in plants, especially in Arabidopsis thaliana, increasing or generating the activity of a gene product non-targeted with the activity of a “methylglyoxal synthase”, preferably being encoded by a gene comprising the nucleic acid sequence SEQ ID NO. 36670, preferably the coding region thereof, conferred the production of or the increase in phenylalanine compared with the wild type control.

The nucleic acid sequence of B0963 from Escherichia coli, e.g. as shown in the respective line in column 5 of Table I, application no. 3, has been published in B. And the activity of the gene product thereof is the activity of methylglyoxal synthase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “methylglyoxal synthase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0963, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0963, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0963, or a functional equivalent or a homolog thereof as depicted in column 8 of Table II, application no. 3, preferably a homolog or functional equivalent as depicted in column 8 of Table II B, application no. 3, and being depicted in the same respective line as said B0963, and preferably the activity is increased non-targeted.

Accordingly, in one embodiment, the molecule which activity is to be increased in the process of the invention is the gene product with an activity as a “methylglyoxal synthase”, preferably it is the molecule of section (a) or (b) of this paragraph.

In particular, it was observed that in plants, especially in Arabidopsis thaliana, increasing or generating the activity of a gene product non-targeted with the activity of a “methylglyoxal synthase”, preferably being encoded by a gene comprising the nucleic acid sequence SEQ ID NO. 36670, preferably the coding region thereof, conferred the production of or the increase in tyrosine compared with the wild type control.

The nucleic acid sequence of B0980 from Escherichia coli, e.g. as shown in the respective line in column 5 of Table I, application no. 3, has been published in B. And the activity of the gene product thereof is the activity of phosphoanhydride phosphorylase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “phosphoanhydride phosphorylase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B0980, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B0980, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B0980, or a functional equivalent or a homolog thereof as depicted in column 8 of Table II, application no. 3, preferably a homolog or functional equivalent as depicted in column 8 of Table II B, application no. 3, and being depicted in the same respective line as said B0980, and preferably the activity is increased non-targeted.

Accordingly, in one embodiment, the molecule which activity is to be increased in the process of the invention is the gene product with an activity as a “phosphoanhydride phosphorylase”, preferably it is the molecule of section (a) or (b) of this paragraph.

In particular, it was observed that in plants, especially in Arabidopsis thaliana, increasing or generating the activity of a gene product non-targeted with the activity of a “phosphoanhydride phosphorylase”, preferably being encoded by a gene comprising the nucleic acid sequence SEQ ID NO. 36809, preferably the coding region thereof, conferred the production of or the increase in phenylalanine compared with the wild type control.

The nucleic acid sequence of B1003 from Escherichia coli, e.g. as shown in the respective line in column 5 of Table I, application no. 3, has been published in B. And the activity of the gene product thereof is the activity of b1003-protein.

Accordingly, in one embodiment, the process of the present invention for producing alanine in a non-human organism, like a microorganism or a plant or a part thereof, comprises increasing or generating the activity of a gene product with the activity of a gene product conferring the activity of “b1003-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1003, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1003, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1003, or a functional equivalent or a homolog thereof as depicted in column 8 of Table II, application no. 3, preferably a homolog or functional equivalent as depicted in column 8 of Table II B, application no. 3, and being depicted in the same respective line as said B1003, and preferably the activity is increased non-targeted.

Accordingly, in one embodiment, the molecule which activity is to be increased in the process of the invention is the gene product with an activity as a “b1003-protein”, preferably it is the molecule of section (a) or (b) of this paragraph.

In particular, it was observed that in plants, especially in Arabidopsis thaliana, increasing or generating the activity of a gene product non-targeted with the activity of a “b1003-protein”, preferably being encoded by a gene comprising the nucleic acid sequence SEQ ID NO. 7941, preferably the coding region thereof, conferred the production of or the increase in alanine compared with the wild type control.

The nucleic acid sequence of B1003 from Escherichia coli, e.g. as shown in the respective line in column 5 of Table I, application no. 3, has been published in B. And the activity of the gene product thereof is the activity of b1003-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “b1003-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1003, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1003, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1003, or a functional equivalent or a homolog thereof as depicted in column 8 of Table II, application no. 3, preferably a homolog or functional equivalent as depicted in column 8 of Table II B, application no. 3, and being depicted in the same respective line as said B1003, and preferably the activity is increased non-targeted.

Accordingly, in one embodiment, the molecule which activity is to be increased in the process of the invention is the gene product with an activity as a “b1003-protein”, preferably it is the molecule of section (a) or (b) of this paragraph.

In particular, it was observed that in plants, especially in Arabidopsis thaliana, increasing or generating the activity of a gene product non-targeted with the activity of a “b1003-protein”, preferably being encoded by a gene comprising the nucleic acid sequence SEQ ID NO. 7941, preferably the coding region thereof, conferred the production of or the increase in aspartate compared with the wild type control.

The nucleic acid sequence of B1003 from Escherichia coli, e.g. as shown in the respective line in column 5 of Table I, application no. 3, has been published in B. And the activity of the gene product thereof is the activity of b1003-protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “b1003-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1003, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1003, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1003, or a functional equivalent or a homolog thereof as depicted in column 8 of Table II, application no. 3, preferably a homolog or functional equivalent as depicted in column 8 of Table II B, application no. 3, and being depicted in the same respective line as said B1003, and preferably the activity is increased non-targeted.

Accordingly, in one embodiment, the molecule which activity is to be increased in the process of the invention is the gene product with an activity as a “b1003-protein”, preferably it is the molecule of section (a) or (b) of this paragraph.

In particular, it was observed that in plants, especially in Arabidopsis thaliana, increasing or generating the activity of a gene product non-targeted with the activity of a “b1003-protein”, preferably being encoded by a gene comprising the nucleic acid sequence SEQ ID NO. 7941, preferably the coding region thereof, conferred the production of or the increase in phenylalanine compared with the wild type control.

The nucleic acid sequence of B1003 from Escherichia coli, e.g. as shown in the respective line in column 5 of Table I, application no. 3, has been published in B. And the activity of the gene product thereof is the activity of b1003-protein.

Accordingly, in one embodiment, the process of the present invention for producing serine in a non-human organism, like a microorganism or a plant or a part thereof, comprises increasing or generating the activity of a gene product with the activity of a gene product conferring the activity of “b1003-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1003, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1003, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1003, or a functional equivalent or a homolog thereof as depicted in column 8 of Table II, application no. 3, preferably a homolog or functional equivalent as depicted in column 8 of Table II B, application no. 3, and being depicted in the same respective line as said B1003, and preferably the activity is increased non-targeted.

Accordingly, in one embodiment, the molecule which activity is to be increased in the process of the invention is the gene product with an activity as a “b1003-protein”, preferably it is the molecule of section (a) or (b) of this paragraph.

In particular, it was observed that in plants, especially in Arabidopsis thaliana, increasing or generating the activity of a gene product non-targeted with the activity of a “b1003-protein”, preferably being encoded by a gene comprising the nucleic acid sequence SEQ ID NO. 7941, preferably the coding region thereof, conferred the production of or the increase in serine compared with the wild type control.

The nucleic acid sequence of B1003 from Escherichia coli, e.g. as shown in the respective line in column 5 of Table I, application no. 3, has been published in B. And the activity of the gene product thereof is the activity of b1003-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “b1003-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1003, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1003, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1003, or a functional equivalent or a homolog thereof as depicted in column 8 of Table II, application no. 3, preferably a homolog or functional equivalent as depicted in column 8 of Table II B, application no. 3, and being depicted in the same respective line as said B1003, and preferably the activity is increased non-targeted.

Accordingly, in one embodiment, the molecule which activity is to be increased in the process of the invention is the gene product with an activity as a “b1003-protein”, preferably it is the molecule of section (a) or (b) of this paragraph.

In particular, it was observed that in plants, especially in Arabidopsis thaliana, increasing or generating the activity of a gene product non-targeted with the activity of a “b1003-protein”, preferably being encoded by a gene comprising the nucleic acid sequence SEQ ID NO. 7941, preferably the coding region thereof, conferred the production of or the increase in tyrosine compared with the wild type control.

The nucleic acid sequence of B1003 from Escherichia coli, e.g. as shown in the respective line in column 5 of Table I, application no. 3, has been published in B. And the activity of the gene product thereof is the activity of b1003-protein.

Accordingly, in one embodiment, the process of the present invention for producing 3,4-dihydroxyphenylalanine (dopa) in a non-human organism, like a microorganism or a plant or a part thereof, comprises increasing or generating the activity of a gene product with the activity of a gene product conferring the activity of “b1003-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1003, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1003, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1003, or a functional equivalent or a homolog thereof as depicted in column 8 of Table II, application no. 3, preferably a homolog or functional equivalent as depicted in column 8 of Table II B, application no. 3, and being depicted in the same respective line as said B1003, and preferably the activity is increased non-targeted.

Accordingly, in one embodiment, the molecule which activity is to be increased in the process of the invention is the gene product with an activity as a “b1003-protein”, preferably it is the molecule of section (a) or (b) of this paragraph.

In particular, it was observed that in plants, especially in Arabidopsis thaliana, increasing or generating the activity of a gene product non-targeted with the activity of a “b1003-protein”, preferably being encoded by a gene comprising the nucleic acid sequence SEQ ID NO. 7941, preferably the coding region thereof, conferred the production of or the increase in 3,4-dihydroxyphenylalanine (dopa) compared with the wild type control.

The nucleic acid sequence of B1003 from Escherichia coli, e.g. as shown in the respective line in column 5 of Table I, application no. 3, has been published in B. And the activity of the gene product thereof is the activity of b1003-protein.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “b1003-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1003, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1003, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1003, or a functional equivalent or a homolog thereof as depicted in column 8 of Table II, application no. 3, preferably a homolog or functional equivalent as depicted in column 8 of Table II B, application no. 3, and being depicted in the same respective line as said B1003, and preferably the activity is increased non-targeted.

Accordingly, in one embodiment, the molecule which activity is to be increased in the process of the invention is the gene product with an activity as a “b1003-protein”, preferably it is the molecule of section (a) or (b) of this paragraph.

In particular, it was observed that in plants, especially in Arabidopsis thaliana, increasing or generating the activity of a gene product non-targeted with the activity of a “b1003-protein”, preferably being encoded by a gene comprising the nucleic acid sequence SEQ ID NO. 7941, preferably the coding region thereof, conferred the production of or the increase in 5-oxoproline compared with the wild type control.

The nucleic acid sequence of B1023 from Escherichia coli, e.g. as shown in the respective line in column 5 of Table I, application no. 3, has been published in B. And the activity of the gene product thereof is the activity of lipoprotein precursor.

Accordingly, in one embodiment, the process of the present invention for producing citrulline in a non-human organism, like a microorganism or a plant or a part thereof, comprises increasing or generating the activity of a gene product with the activity of a gene product conferring the activity of “lipoprotein precursor”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1023, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1023, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1023, or a functional equivalent or a homolog thereof as depicted in column 8 of Table II, application no. 3, preferably a homolog or functional equivalent as depicted in column 8 of Table II B, application no. 3, and being depicted in the same respective line as said B1023, and preferably the activity is increased non-targeted.

Accordingly, in one embodiment, the molecule which activity is to be increased in the process of the invention is the gene product with an activity as a “lipoprotein precursor”, preferably it is the molecule of section (a) or (b) of this paragraph.

In particular, it was observed that in plants, especially in Arabidopsis thaliana, increasing or generating the activity of a gene product non-targeted with the activity of a “lipoprotein precursor”, preferably being encoded by a gene comprising the nucleic acid sequence SEQ ID NO. 36880, preferably the coding region thereof, conferred the production of or the increase in citrulline compared with the wild type control.

The nucleic acid sequence of B1023 from Escherichia coli, e.g. as shown in the respective line in column 5 of Table I, application no. 3, has been published in B. And the activity of the gene product thereof is the activity of lipoprotein precursor.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “lipoprotein precursor”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1023, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1023, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1023, or a functional equivalent or a homolog thereof as depicted in column 8 of Table II, application no. 3, preferably a homolog or functional equivalent as depicted in column 8 of Table II B, application no. 3, and being depicted in the same respective line as said B1023, and preferably the activity is increased non-targeted.

Accordingly, in one embodiment, the molecule which activity is to be increased in the process of the invention is the gene product with an activity as a “lipoprotein precursor”, preferably it is the molecule of section (a) or (b) of this paragraph.

In particular, it was observed that in plants, especially in Arabidopsis thaliana, increasing or generating the activity of a gene product non-targeted with the activity of a “lipoprotein precursor”, preferably being encoded by a gene comprising the nucleic acid sequence SEQ ID NO. 36880, preferably the coding region thereof, conferred the production of or the increase in 5-oxoproline compared with the wild type control.

The nucleic acid sequence of B1024 from Escherichia coli, e.g. as shown in the respective line in column 5 of Table I, application no. 3, has been published in B. And the activity of the gene product thereof is the activity of b1024-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “b1024-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1024, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1024, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1024, or a functional equivalent or a homolog thereof as depicted in column 8 of Table II, application no. 3, preferably a homolog or functional equivalent as depicted in column 8 of Table II B, application no. 3, and being depicted in the same respective line as said B1024, and preferably the activity is increased non-targeted.

Accordingly, in one embodiment, the molecule which activity is to be increased in the process of the invention is the gene product with an activity as a “b1024-protein”, preferably it is the molecule of section (a) or (b) of this paragraph.

In particular, it was observed that in plants, especially in Arabidopsis thaliana, increasing or generating the activity of a gene product non-targeted with the activity of a “b1024-protein”, preferably being encoded by a gene comprising the nucleic acid sequence SEQ ID NO. 36907, preferably the coding region thereof, conferred the production of or the increase in aspartate compared with the wild type control.

The nucleic acid sequence of B1065 from Escherichia coli, e.g. as shown in the respective line in column 5 of Table I, application no. 3, has been published in B. And the activity of the gene product thereof is the activity of multidrug resistance protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “multidrug resistance protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1065, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1065, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1065, or a functional equivalent or a homolog thereof as depicted in column 8 of Table II, application no. 3, preferably a homolog or functional equivalent as depicted in column 8 of Table II B, application no. 3, and being depicted in the same respective line as said B1065, and preferably the activity is increased plastidic.

Accordingly, in one embodiment, the molecule which activity is to be increased in the process of the invention is the gene product with an activity as a “multidrug resistance protein”, preferably it is the molecule of section (a) or (b) of this paragraph.

In particular, it was observed that in plants, especially in Arabidopsis thaliana, increasing or generating the activity of a gene product plastidic with the activity of a “multidrug resistance protein”, preferably being encoded by a gene comprising the nucleic acid sequence SEQ ID NO. 76032, preferably the coding region thereof, conferred the production of or the increase in aspartate compared with the wild type control.

The nucleic acid sequence of B1065 from Escherichia coli, e.g. as shown in the respective line in column 5 of Table I, application no. 3, has been published in B. And the activity of the gene product thereof is the activity of multidrug resistance protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “multidrug resistance protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1065, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1065, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1065, or a functional equivalent or a homolog thereof as depicted in column 8 of Table II, application no. 3, preferably a homolog or functional equivalent as depicted in column 8 of Table II B, application no. 3, and being depicted in the same respective line as said B1065, and preferably the activity is increased plastidic.

Accordingly, in one embodiment, the molecule which activity is to be increased in the process of the invention is the gene product with an activity as a “multidrug resistance protein”, preferably it is the molecule of section (a) or (b) of this paragraph.

In particular, it was observed that in plants, especially in Arabidopsis thaliana, increasing or generating the activity of a gene product plastidic with the activity of a “multidrug resistance protein”, preferably being encoded by a gene comprising the nucleic acid sequence SEQ ID NO. 76032, preferably the coding region thereof, conferred the production of or the increase in tyrosine compared with the wild type control.

The nucleic acid sequence of B1137 from Escherichia coli, e.g. as shown in the respective line in column 5 of Table I, application no. 3, has been published in B. And the activity of the gene product thereof is the activity of b1137-protein.

Accordingly, in one embodiment, the process of the present invention for producing serine in a non-human organism, like a microorganism or a plant or a part thereof, comprises increasing or generating the activity of a gene product with the activity of a gene product conferring the activity of “b1137-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1137, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1137, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1137, or a functional equivalent or a homolog thereof as depicted in column 8 of Table II, application no. 3, preferably a homolog or functional equivalent as depicted in column 8 of Table II B, application no. 3, and being depicted in the same respective line as said B1137, and preferably the activity is increased non-targeted.

Accordingly, in one embodiment, the molecule which activity is to be increased in the process of the invention is the gene product with an activity as a “b1137-protein”, preferably it is the molecule of section (a) or (b) of this paragraph.

In particular, it was observed that in plants, especially in Arabidopsis thaliana, increasing or generating the activity of a gene product non-targeted with the activity of a “b1137-protein”, preferably being encoded by a gene comprising the nucleic acid sequence SEQ ID NO. 37390, preferably the coding region thereof, conferred the production of or the increase in serine compared with the wild type control.

The nucleic acid sequence of B1155 from Escherichia coli, e.g. as shown in the respective line in column 5 of Table I, application no. 3, has been published in B. And the activity of the gene product thereof is the activity of b1155-protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “b1155-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1155, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1155, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1155, or a functional equivalent or a homolog thereof as depicted in column 8 of Table II, application no. 3, preferably a homolog or functional equivalent as depicted in column 8 of Table II B, application no. 3, and being depicted in the same respective line as said B1155, and preferably the activity is increased non-targeted.

Accordingly, in one embodiment, the molecule which activity is to be increased in the process of the invention is the gene product with an activity as a “b1155-protein”, preferably it is the molecule of section (a) or (b) of this paragraph.

In particular, it was observed that in plants, especially in Arabidopsis thaliana, increasing or generating the activity of a gene product non-targeted with the activity of a “b1155-protein”, preferably being encoded by a gene comprising the nucleic acid sequence SEQ ID NO. 76068, preferably the coding region thereof, conferred the production of or the increase in phenylalanine compared with the wild type control.

The nucleic acid sequence of B1163 from Escherichia coli, e.g. as shown in the respective line in column 5 of Table I, application no. 3, has been published in B. And the activity of the gene product thereof is the activity of b1163-protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “b1163-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1163, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1163, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1163, or a functional equivalent or a homolog thereof as depicted in column 8 of Table II, application no. 3, preferably a homolog or functional equivalent as depicted in column 8 of Table II B, application no. 3, and being depicted in the same respective line as said B1163, and preferably the activity is increased non-targeted.

Accordingly, in one embodiment, the molecule which activity is to be increased in the process of the invention is the gene product with an activity as a “b1163-protein”, preferably it is the molecule of section (a) or (b) of this paragraph.

In particular, it was observed that in plants, especially in Arabidopsis thaliana, increasing or generating the activity of a gene product non-targeted with the activity of a “b1163-protein”, preferably being encoded by a gene comprising the nucleic acid sequence SEQ ID NO. 37394, preferably the coding region thereof, conferred the production of or the increase in phenylalanine compared with the wild type control.

The nucleic acid sequence of B1163 from Escherichia coli, e.g. as shown in the respective line in column 5 of Table I, application no. 3, has been published in B. And the activity of the gene product thereof is the activity of b1163-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “b1163-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1163, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1163, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1163, or a functional equivalent or a homolog thereof as depicted in column 8 of Table II, application no. 3, preferably a homolog or functional equivalent as depicted in column 8 of Table II B, application no. 3, and being depicted in the same respective line as said B1163, and preferably the activity is increased non-targeted.

Accordingly, in one embodiment, the molecule which activity is to be increased in the process of the invention is the gene product with an activity as a “b1163-protein”, preferably it is the molecule of section (a) or (b) of this paragraph.

In particular, it was observed that in plants, especially in Arabidopsis thaliana, increasing or generating the activity of a gene product non-targeted with the activity of a “b1163-protein”, preferably being encoded by a gene comprising the nucleic acid sequence SEQ ID NO. 37394, preferably the coding region thereof, conferred the production of or the increase in tyrosine compared with the wild type control.

The nucleic acid sequence of B1179 from Escherichia coli, e.g. as shown in the respective line in column 5 of Table I, application no. 3, has been published in B. And the activity of the gene product thereof is the activity of b1179-protein.

Accordingly, in one embodiment, the process of the present invention for producing citrulline in a non-human organism, like a microorganism or a plant or a part thereof, comprises increasing or generating the activity of a gene product with the activity of a gene product conferring the activity of “b1179-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1179, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1179, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1179, or a functional equivalent or a homolog thereof as depicted in column 8 of Table II, application no. 3, preferably a homolog or functional equivalent as depicted in column 8 of Table II B, application no. 3, and being depicted in the same respective line as said B1179, and preferably the activity is increased plastidic.

Accordingly, in one embodiment, the molecule which activity is to be increased in the process of the invention is the gene product with an activity as a “b1179-protein”, preferably it is the molecule of section (a) or (b) of this paragraph.

In particular, it was observed that in plants, especially in Arabidopsis thaliana, increasing or generating the activity of a gene product plastidic with the activity of a “b1179-protein”, preferably being encoded by a gene comprising the nucleic acid sequence SEQ ID NO. 76087, preferably the coding region thereof, conferred the production of or the increase in citrulline compared with the wild type control.

The nucleic acid sequence of B1186 from Escherichia coli K12, e.g. as shown in the respective line in column 5 of Table I, application no. 3, has been published in B. And the activity of the gene product thereof is the activity of sodium/proton antiporter.

Accordingly, in one embodiment, the process of the present invention for producing alanine in a non-human organism, like a microorganism or a plant or a part thereof, comprises increasing or generating the activity of a gene product with the activity of a gene product conferring the activity of “sodium/proton antiporter”, especially from Escherichia coli K12 or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1186, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1186, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1186, or a functional equivalent or a homolog thereof as depicted in column 8 of Table II, application no. 3, preferably a homolog or functional equivalent as depicted in column 8 of Table II B, application no. 3, and being depicted in the same respective line as said B1186, and preferably the activity is increased non-targeted.

Accordingly, in one embodiment, the molecule which activity is to be increased in the process of the invention is the gene product with an activity as a “sodium/proton antiporter”, preferably it is the molecule of section (a) or (b) of this paragraph.

In particular, it was observed that in plants, especially in Arabidopsis thaliana, increasing or generating the activity of a gene product non-targeted with the activity of a “sodium/proton antiporter”, preferably being encoded by a gene comprising the nucleic acid sequence SEQ ID NO. 37400, preferably the coding region thereof, conferred the production of or the increase in alanine compared with the wild type control.

The nucleic acid sequence of B1186 from Escherichia coli K12, e.g. as shown in the respective line in column 5 of Table I, application no. 3, has been published in B. And the activity of the gene product thereof is the activity of sodium/proton antiporter.

Accordingly, in one embodiment, the process of the present invention for producing serine in a non-human organism, like a microorganism or a plant or a part thereof, comprises increasing or generating the activity of a gene product with the activity of a gene product conferring the activity of “sodium/proton antiporter”, especially from Escherichia coli K12 or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1186, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1186, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1186, or a functional equivalent or a homolog thereof as depicted in column 8 of Table II, application no. 3, preferably a homolog or functional equivalent as depicted in column 8 of Table II B, application no. 3, and being depicted in the same respective line as said B1186, and preferably the activity is increased non-targeted.

Accordingly, in one embodiment, the molecule which activity is to be increased in the process of the invention is the gene product with an activity as a “sodium/proton antiporter”, preferably it is the molecule of section (a) or (b) of this paragraph.

In particular, it was observed that in plants, especially in Arabidopsis thaliana, increasing or generating the activity of a gene product non-targeted with the activity of a “sodium/proton antiporter”, preferably being encoded by a gene comprising the nucleic acid sequence SEQ ID NO. 37400, preferably the coding region thereof, conferred the production of or the increase in serine compared with the wild type control.

The nucleic acid sequence of B1205 from Escherichia coli, e.g. as shown in the respective line in column 5 of Table I, application no. 3, has been published in B. And the activity of the gene product thereof is the activity of b1205-protein.

Accordingly, in one embodiment, the process of the present invention for producing citrulline in a non-human organism, like a microorganism or a plant or a part thereof, comprises increasing or generating the activity of a gene product with the activity of a gene product conferring the activity of “b1205-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1205, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1205, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1205, or a functional equivalent or a homolog thereof as depicted in column 8 of Table II, application no. 3, preferably a homolog or functional equivalent as depicted in column 8 of Table II B, application no. 3, and being depicted in the same respective line as said B1205, and preferably the activity is increased non-targeted.

Accordingly, in one embodiment, the molecule which activity is to be increased in the process of the invention is the gene product with an activity as a “b1205-protein”, preferably it is the molecule of section (a) or (b) of this paragraph.

In particular, it was observed that in plants, especially in Arabidopsis thaliana, increasing or generating the activity of a gene product non-targeted with the activity of a “b1205-protein”, preferably being encoded by a gene comprising the nucleic acid sequence SEQ ID NO. 76138, preferably the coding region thereof, conferred the production of or the increase in citrulline compared with the wild type control.

The nucleic acid sequence of B1229 from Escherichia coli, e.g. as shown in the respective line in column 5 of Table I, application no. 3, has been published in B. And the activity of the gene product thereof is the activity of b1229-protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “b1229-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1229, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1229, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1229, or a functional equivalent or a homolog thereof as depicted in column 8 of Table II, application no. 3, preferably a homolog or functional equivalent as depicted in column 8 of Table II B, application no. 3, and being depicted in the same respective line as said B1229, and preferably the activity is increased non-targeted.

Accordingly, in one embodiment, the molecule which activity is to be increased in the process of the invention is the gene product with an activity as a “b1229-protein”, preferably it is the molecule of section (a) or (b) of this paragraph.

In particular, it was observed that in plants, especially in Arabidopsis thaliana, increasing or generating the activity of a gene product non-targeted with the activity of a “b1229-protein”, preferably being encoded by a gene comprising the nucleic acid sequence SEQ ID NO. 76151, preferably the coding region thereof, conferred the production of or the increase in phenylalanine compared with the wild type control.

The nucleic acid sequence of B1255 from Escherichia coli, e.g. as shown in the respective line in column 5 of Table I, application no. 3, has been published in B. And the activity of the gene product thereof is the activity of membrane protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “membrane protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1255, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1255, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1255, or a functional equivalent or a homolog thereof as depicted in column 8 of Table II, application no. 3, preferably a homolog or functional equivalent as depicted in column 8 of Table II B, application no. 3, and being depicted in the same respective line as said B1255, and preferably the activity is increased non-targeted.

Accordingly, in one embodiment, the molecule which activity is to be increased in the process of the invention is the gene product with an activity as a “membrane protein”, preferably it is the molecule of section (a) or (b) of this paragraph.

In particular, it was observed that in plants, especially in Arabidopsis thaliana, increasing or generating the activity of a gene product non-targeted with the activity of a “membrane protein”, preferably being encoded by a gene comprising the nucleic acid sequence SEQ ID NO. 37483, preferably the coding region thereof, conferred the production of or the increase in tyrosine compared with the wild type control.

The nucleic acid sequence of B1259 from Escherichia coli, e.g. as shown in the respective line in column 5 of Table I, application no. 3, has been published in B. And the activity of the gene product thereof is the activity of b1259-protein.

Accordingly, in one embodiment, the process of the present invention for producing citrulline in a non-human organism, like a microorganism or a plant or a part thereof, comprises increasing or generating the activity of a gene product with the activity of a gene product conferring the activity of “b1259-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1259, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1259, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1259, or a functional equivalent or a homolog thereof as depicted in column 8 of Table II, application no. 3, preferably a homolog or functional equivalent as depicted in column 8 of Table II B, application no. 3, and being depicted in the same respective line as said B1259, and preferably the activity is increased non-targeted.

Accordingly, in one embodiment, the molecule which activity is to be increased in the process of the invention is the gene product with an activity as a “b1259-protein”, preferably it is the molecule of section (a) or (b) of this paragraph.

In particular, it was observed that in plants, especially in Arabidopsis thaliana, increasing or generating the activity of a gene product non-targeted with the activity of a “b1259-protein”, preferably being encoded by a gene comprising the nucleic acid sequence SEQ ID NO. 37503, preferably the coding region thereof, conferred the production of or the increase in citrulline compared with the wild type control.

The nucleic acid sequence of B1259 from Escherichia coli, e.g. as shown in the respective line in column 5 of Table I, application no. 3, has been published in B. And the activity of the gene product thereof is the activity of b1259-protein.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “b1259-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1259, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1259, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1259, or a functional equivalent or a homolog thereof as depicted in column 8 of Table II, application no. 3, preferably a homolog or functional equivalent as depicted in column 8 of Table II B, application no. 3, and being depicted in the same respective line as said B1259, and preferably the activity is increased non-targeted.

Accordingly, in one embodiment, the molecule which activity is to be increased in the process of the invention is the gene product with an activity as a “b1259-protein”, preferably it is the molecule of section (a) or (b) of this paragraph.

In particular, it was observed that in plants, especially in Arabidopsis thaliana, increasing or generating the activity of a gene product non-targeted with the activity of a “b1259-protein”, preferably being encoded by a gene comprising the nucleic acid sequence SEQ ID NO. 37503, preferably the coding region thereof, conferred the production of or the increase in 5-oxoproline compared with the wild type control.

The nucleic acid sequence of B1262 from Escherichia coli, e.g. as shown in the respective line in column 5 of Table I, application no. 3, has been published in B. And the activity of the gene product thereof is the activity of tryptophan biosynthesis protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “tryptophan biosynthesis protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1262, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1262, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1262, or a functional equivalent or a homolog thereof as depicted in column 8 of Table II, application no. 3, preferably a homolog or functional equivalent as depicted in column 8 of Table II B, application no. 3, and being depicted in the same respective line as said B1262, and preferably the activity is increased plastidic.

Accordingly, in one embodiment, the molecule which activity is to be increased in the process of the invention is the gene product with an activity as a “tryptophan biosynthesis protein”, preferably it is the molecule of section (a) or (b) of this paragraph.

In particular, it was observed that in plants, especially in Arabidopsis thaliana, increasing or generating the activity of a gene product plastidic with the activity of a “tryptophan biosynthesis protein”, preferably being encoded by a gene comprising the nucleic acid sequence SEQ ID NO. 76157, preferably the coding region thereof, conferred the production of or the increase in tyrosine compared with the wild type control.

The nucleic acid sequence of B1264 from Escherichia coli, e.g. as shown in the respective line in column 5 of Table I, application no. 3, has been published in B. And the activity of the gene product thereof is the activity of anthranilate synthase component I.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “anthranilate synthase component I”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1264, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1264, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1264, or a functional equivalent or a homolog thereof as depicted in column 8 of Table II, application no. 3, preferably a homolog or functional equivalent as depicted in column 8 of Table II B, application no. 3, and being depicted in the same respective line as said B1264, and preferably the activity is increased plastidic.

Accordingly, in one embodiment, the molecule which activity is to be increased in the process of the invention is the gene product with an activity as a “anthranilate synthase component I”, preferably it is the molecule of section (a) or (b) of this paragraph.

In particular, it was observed that in plants, especially in Arabidopsis thaliana, increasing or generating the activity of a gene product plastidic with the activity of a “anthranilate synthase component I”, preferably being encoded by a gene comprising the nucleic acid sequence SEQ ID NO. 76240, preferably the coding region thereof, conferred the production of or the increase in phenylalanine compared with the wild type control.

The nucleic acid sequence of B1280 from Escherichia coli, e.g. as shown in the respective line in column 5 of Table I, application no. 3, has been published in B. And the activity of the gene product thereof is the activity of b1280-protein.

Accordingly, in one embodiment, the process of the present invention for producing glycine in a non-human organism, like a microorganism or a plant or a part thereof, comprises increasing or generating the activity of a gene product with the activity of a gene product conferring the activity of “b1280-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1280, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1280, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1280, or a functional equivalent or a homolog thereof as depicted in column 8 of Table II, application no. 3, preferably a homolog or functional equivalent as depicted in column 8 of Table II B, application no. 3, and being depicted in the same respective line as said B1280, and preferably the activity is increased non-targeted.

Accordingly, in one embodiment, the molecule which activity is to be increased in the process of the invention is the gene product with an activity as a “b1280-protein”, preferably it is the molecule of section (a) or (b) of this paragraph.

In particular, it was observed that in plants, especially in Arabidopsis thaliana, increasing or generating the activity of a gene product non-targeted with the activity of a “b1280-protein”, preferably being encoded by a gene comprising the nucleic acid sequence SEQ ID NO. 37573, preferably the coding region thereof, conferred the production of or the increase in glycine compared with the wild type control.

The nucleic acid sequence of B1280 from Escherichia coli, e.g. as shown in the respective line in column 5 of Table I, application no. 3, has been published in B. And the activity of the gene product thereof is the activity of b1280-protein.

Accordingly, in one embodiment, the process of the present invention for producing serine in a non-human organism, like a microorganism or a plant or a part thereof, comprises increasing or generating the activity of a gene product with the activity of a gene product conferring the activity of “b1280-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1280, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1280, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1280, or a functional equivalent or a homolog thereof as depicted in column 8 of Table II, application no. 3, preferably a homolog or functional equivalent as depicted in column 8 of Table II B, application no. 3, and being depicted in the same respective line as said B1280, and preferably the activity is increased non-targeted.

Accordingly, in one embodiment, the molecule which activity is to be increased in the process of the invention is the gene product with an activity as a “b1280-protein”, preferably it is the molecule of section (a) or (b) of this paragraph.

In particular, it was observed that in plants, especially in Arabidopsis thaliana, increasing or generating the activity of a gene product non-targeted with the activity of a “b1280-protein”, preferably being encoded by a gene comprising the nucleic acid sequence SEQ ID NO. 37573, preferably the coding region thereof, conferred the production of or the increase in serine compared with the wild type control.

The nucleic acid sequence of B1280 from Escherichia coli, e.g. as shown in the respective line in column 5 of Table I, application no. 3, has been published in B. And the activity of the gene product thereof is the activity of b1280-protein.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “b1280-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1280, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1280, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1280, or a functional equivalent or a homolog thereof as depicted in column 8 of Table II, application no. 3, preferably a homolog or functional equivalent as depicted in column 8 of Table II B, application no. 3, and being depicted in the same respective line as said B1280, and preferably the activity is increased non-targeted.

Accordingly, in one embodiment, the molecule which activity is to be increased in the process of the invention is the gene product with an activity as a “b1280-protein”, preferably it is the molecule of section (a) or (b) of this paragraph.

In particular, it was observed that in plants, especially in Arabidopsis thaliana, increasing or generating the activity of a gene product non-targeted with the activity of a “b1280-protein”, preferably being encoded by a gene comprising the nucleic acid sequence SEQ ID NO. 37573, preferably the coding region thereof, conferred the production of or the increase in 5-oxoproline compared with the wild type control.

The nucleic acid sequence of B1297 from Escherichia coli, e.g. as shown in the respective line in column 5 of Table I, application no. 3, has been published in B. And the activity of the gene product thereof is the activity of glutamine synthetase.

Accordingly, in one embodiment, the process of the present invention for producing alanine in a non-human organism, like a microorganism or a plant or a part thereof, comprises increasing or generating the activity of a gene product with the activity of a gene product conferring the activity of “glutamine synthetase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1297, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1297, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1297, or a functional equivalent or a homolog thereof as depicted in column 8 of Table II, application no. 3, preferably a homolog or functional equivalent as depicted in column 8 of Table II B, application no. 3, and being depicted in the same respective line as said B1297, and preferably the activity is increased plastidic.

Accordingly, in one embodiment, the molecule which activity is to be increased in the process of the invention is the gene product with an activity as a “glutamine synthetase”, preferably it is the molecule of section (a) or (b) of this paragraph.

In particular, it was observed that in plants, especially in Arabidopsis thaliana, increasing or generating the activity of a gene product plastidic with the activity of a “glutamine synthetase”, preferably being encoded by a gene comprising the nucleic acid sequence SEQ ID NO. 37658, preferably the coding region thereof, conferred the production of or the increase in alanine compared with the wild type control.

The nucleic acid sequence of B1297 from Escherichia coli, e.g. as shown in the respective line in column 5 of Table I, application no. 3, has been published in B. And the activity of the gene product thereof is the activity of glutamine synthetase.

Accordingly, in one embodiment, the process of the present invention for producing glycine in a non-human organism, like a microorganism or a plant or a part thereof, comprises increasing or generating the activity of a gene product with the activity of a gene product conferring the activity of “glutamine synthetase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1297, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1297, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1297, or a functional equivalent or a homolog thereof as depicted in column 8 of Table II, application no. 3, preferably a homolog or functional equivalent as depicted in column 8 of Table II B, application no. 3, and being depicted in the same respective line as said B1297, and preferably the activity is increased plastidic.

Accordingly, in one embodiment, the molecule which activity is to be increased in the process of the invention is the gene product with an activity as a “glutamine synthetase”, preferably it is the molecule of section (a) or (b) of this paragraph.

In particular, it was observed that in plants, especially in Arabidopsis thaliana, increasing or generating the activity of a gene product plastidic with the activity of a “glutamine synthetase”, preferably being encoded by a gene comprising the nucleic acid sequence SEQ ID NO. 37658, preferably the coding region thereof, conferred the production of or the increase in glycine compared with the wild type control.

The nucleic acid sequence of B1300 from Escherichia coli, e.g. as shown in the respective line in column 5 of Table I, application no. 3, has been published in B. And the activity of the gene product thereof is the activity of aldehyde dehydrogenase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “aldehyde dehydrogenase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1300, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1300, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1300, or a functional equivalent or a homolog thereof as depicted in column 8 of Table II, application no. 3, preferably a homolog or functional equivalent as depicted in column 8 of Table II B, application no. 3, and being depicted in the same respective line as said B1300, and preferably the activity is increased non-targeted.

Accordingly, in one embodiment, the molecule which activity is to be increased in the process of the invention is the gene product with an activity as a “aldehyde dehydrogenase”, preferably it is the molecule of section (a) or (b) of this paragraph.

In particular, it was observed that in plants, especially in Arabidopsis thaliana, increasing or generating the activity of a gene product non-targeted with the activity of a “aldehyde dehydrogenase”, preferably being encoded by a gene comprising the nucleic acid sequence SEQ ID NO. 37807, preferably the coding region thereof, conferred the production of or the increase in phenylalanine compared with the wild type control.

The nucleic acid sequence of B1300 from Escherichia coli, e.g. as shown in the respective line in column 5 of Table I, application no. 3, has been published in B. And the activity of the gene product thereof is the activity of aldehyde dehydrogenase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “aldehyde dehydrogenase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1300, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1300, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1300, or a functional equivalent or a homolog thereof as depicted in column 8 of Table II, application no. 3, preferably a homolog or functional equivalent as depicted in column 8 of Table II B, application no. 3, and being depicted in the same respective line as said B1300, and preferably the activity is increased non-targeted.

Accordingly, in one embodiment, the molecule which activity is to be increased in the process of the invention is the gene product with an activity as a “aldehyde dehydrogenase”, preferably it is the molecule of section (a) or (b) of this paragraph.

In particular, it was observed that in plants, especially in Arabidopsis thaliana, increasing or generating the activity of a gene product non-targeted with the activity of a “aldehyde dehydrogenase”, preferably being encoded by a gene comprising the nucleic acid sequence SEQ ID NO. 37807, preferably the coding region thereof, conferred the production of or the increase in tyrosine compared with the wild type control.

The nucleic acid sequence of B1310 from Escherichia coli, e.g. as shown in the respective line in column 5 of Table I, application no. 3, has been published in B. And the activity of the gene product thereof is the activity of ABC transporter substrate-binding protein.

Accordingly, in one embodiment, the process of the present invention for producing citrulline in a non-human organism, like a microorganism or a plant or a part thereof, comprises increasing or generating the activity of a gene product with the activity of a gene product conferring the activity of “ABC transporter substrate-binding protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1310, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1310, or a functional equivalent or a homolog thereof as depicted in column 8 of Table II, application no. 3, preferably a homolog or functional equivalent as depicted in column 8 of Table II B, application no. 3, and being depicted in the same respective line as said B1310, and preferably the activity is increased non-targeted.

Accordingly, in one embodiment, the molecule which activity is to be increased in the process of the invention is the gene product with an activity as a “ABC transporter substrate-binding protein”, preferably it is the molecule of section (a) or (b) of this paragraph.

In particular, it was observed that in plants, especially in Arabidopsis thaliana, increasing or generating the activity of a gene product non-targeted with the activity of a “ABC transporter substrate-binding protein”, preferably being encoded by a gene comprising the nucleic acid sequence SEQ ID NO. 76867, preferably the coding region thereof, conferred the production of or the increase in citrulline compared with the wild type control.

The nucleic acid sequence of B1330 from Escherichia coli, e.g. as shown in the respective line in column 5 of Table I, application no. 3, has been published in B. And the activity of the gene product thereof is the activity of b1330-protein.

Accordingly, in one embodiment, the process of the present invention for producing citrulline in a non-human organism, like a microorganism or a plant or a part thereof, comprises increasing or generating the activity of a gene product with the activity of a gene product conferring the activity of “b1330-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1330, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1330, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1330, or a functional equivalent or a homolog thereof as depicted in column 8 of Table II, application no. 3, preferably a homolog or functional equivalent as depicted in column 8 of Table II B, application no. 3, and being depicted in the same respective line as said B1330, and preferably the activity is increased non-targeted.

Accordingly, in one embodiment, the molecule which activity is to be increased in the process of the invention is the gene product with an activity as a “b1330-protein”, preferably it is the molecule of section (a) or (b) of this paragraph.

In particular, it was observed that in plants, especially in Arabidopsis thaliana, increasing or generating the activity of a gene product non-targeted with the activity of a “b1330-protein”, preferably being encoded by a gene comprising the nucleic acid sequence SEQ ID NO. 38226, preferably the coding region thereof, conferred the production of or the increase in citrulline compared with the wild type control.

The nucleic acid sequence of B1343 from Escherichia coli, e.g. as shown in the respective line in column 5 of Table I, application no. 3, has been published in B. And the activity of the gene product thereof is the activity of ATP-dependent RNA helicase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “ATP-dependent RNA helicase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1343, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1343, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1343, or a functional equivalent or a homolog thereof as depicted in column 8 of Table II, application no. 3, preferably a homolog or functional equivalent as depicted in column 8 of Table II B, application no. 3, and being depicted in the same respective line as said B1343, and preferably the activity is increased non-targeted.

Accordingly, in one embodiment, the molecule which activity is to be increased in the process of the invention is the gene product with an activity as a “ATP-dependent RNA helicase”, preferably it is the molecule of section (a) or (b) of this paragraph.

In particular, it was observed that in plants, especially in Arabidopsis thaliana, increasing or generating the activity of a gene product non-targeted with the activity of a “ATP-dependent RNA helicase”, preferably being encoded by a gene comprising the nucleic acid sequence SEQ ID NO. 76890, preferably the coding region thereof, conferred the production of or the increase in phenylalanine compared with the wild type control.

The nucleic acid sequence of B1348 from Escherichia coli, e.g. as shown in the respective line in column 5 of Table I, application no. 3, has been published in B. And the activity of the gene product thereof is the activity of restriction alleviation protein.

Accordingly, in one embodiment, the process of the present invention for producing alanine in a non-human organism, like a microorganism or a plant or a part thereof, comprises increasing or generating the activity of a gene product with the activity of a gene product conferring the activity of “restriction alleviation protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1348, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1348, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1348, or a functional equivalent or a homolog thereof as depicted in column 8 of Table II, application no. 3, preferably a homolog or functional equivalent as depicted in column 8 of Table II B, application no. 3, and being depicted in the same respective line as said B1348, and preferably the activity is increased non-targeted.

Accordingly, in one embodiment, the molecule which activity is to be increased in the process of the invention is the gene product with an activity as a “restriction alleviation protein”, preferably it is the molecule of section (a) or (b) of this paragraph.

In particular, it was observed that in plants, especially in Arabidopsis thaliana, increasing or generating the activity of a gene product non-targeted with the activity of a “restriction alleviation protein”, preferably being encoded by a gene comprising the nucleic acid sequence SEQ ID NO. 77768, preferably the coding region thereof, conferred the production of or the increase in alanine compared with the wild type control.

The nucleic acid sequence of B1348 from Escherichia coli, e.g. as shown in the respective line in column 5 of Table I, application no. 3, has been published in B. And the activity of the gene product thereof is the activity of restriction alleviation protein.

Accordingly, in one embodiment, the process of the present invention for producing glycine in a non-human organism, like a microorganism or a plant or a part thereof, comprises increasing or generating the activity of a gene product with the activity of a gene product conferring the activity of “restriction alleviation protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1348, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1348, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1348, or a functional equivalent or a homolog thereof as depicted in column 8 of Table II, application no. 3, preferably a homolog or functional equivalent as depicted in column 8 of Table II B, application no. 3, and being depicted in the same respective line as said B1348, and preferably the activity is increased non-targeted.

Accordingly, in one embodiment, the molecule which activity is to be increased in the process of the invention is the gene product with an activity as a “restriction alleviation protein”, preferably it is the molecule of section (a) or (b) of this paragraph.

In particular, it was observed that in plants, especially in Arabidopsis thaliana, increasing or generating the activity of a gene product non-targeted with the activity of a “restriction alleviation protein”, preferably being encoded by a gene comprising the nucleic acid sequence SEQ ID NO. 77768, preferably the coding region thereof, conferred the production of or the increase in glycine compared with the wild type control.

The nucleic acid sequence of B1431 from Escherichia coli, e.g. as shown in the respective line in column 5 of Table I, application no. 3, has been published in B. And the activity of the gene product thereof is the activity of lipoprotein precursor.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “lipoprotein precursor”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1431, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1431, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1431, or a functional equivalent or a homolog thereof as depicted in column 8 of Table II, application no. 3, preferably a homolog or functional equivalent as depicted in column 8 of Table II B, application no. 3, and being depicted in the same respective line as said B1431, and preferably the activity is increased plastidic.

Accordingly, in one embodiment, the molecule which activity is to be increased in the process of the invention is the gene product with an activity as a “lipoprotein precursor”, preferably it is the molecule of section (a) or (b) of this paragraph.

In particular, it was observed that in plants, especially in Arabidopsis thaliana, increasing or generating the activity of a gene product plastidic with the activity of a “lipoprotein precursor”, preferably being encoded by a gene comprising the nucleic acid sequence SEQ ID NO. 38266, preferably the coding region thereof, conferred the production of or the increase in 5-oxoproline compared with the wild type control.

The nucleic acid sequence of B1445 from Escherichia coli, e.g. as shown in the respective line in column 5 of Table I, application no. 3, has been published in B. And the activity of the gene product thereof is the activity of b1445-protein.

Accordingly, in one embodiment, the process of the present invention for producing alanine in a non-human organism, like a microorganism or a plant or a part thereof, comprises increasing or generating the activity of a gene product with the activity of a gene product conferring the activity of “b1445-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1445, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1445, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1445, or a functional equivalent or a homolog thereof as depicted in column 8 of Table II, application no. 3, preferably a homolog or functional equivalent as depicted in column 8 of Table II B, application no. 3, and being depicted in the same respective line as said B1445, and preferably the activity is increased non-targeted.

Accordingly, in one embodiment, the molecule which activity is to be increased in the process of the invention is the gene product with an activity as a “b1445-protein”, preferably it is the molecule of section (a) or (b) of this paragraph.

In particular, it was observed that in plants, especially in Arabidopsis thaliana, increasing or generating the activity of a gene product non-targeted with the activity of a “b1445-protein”, preferably being encoded by a gene comprising the nucleic acid sequence SEQ ID NO. 38289, preferably the coding region thereof, conferred the production of or the increase in alanine compared with the wild type control.

The nucleic acid sequence of B1445 from Escherichia coli, e.g. as shown in the respective line in column 5 of Table I, application no. 3, has been published in B. And the activity of the gene product thereof is the activity of b1445-protein.

Accordingly, in one embodiment, the process of the present invention for producing citrulline in a non-human organism, like a microorganism or a plant or a part thereof, comprises increasing or generating the activity of a gene product with the activity of a gene product conferring the activity of “b1445-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1445, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1445, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1445, or a functional equivalent or a homolog thereof as depicted in column 8 of Table II, application no. 3, preferably a homolog or functional equivalent as depicted in column 8 of Table II B, application no. 3, and being depicted in the same respective line as said B1445, and preferably the activity is increased non-targeted.

Accordingly, in one embodiment, the molecule which activity is to be increased in the process of the invention is the gene product with an activity as a “b1445-protein”, preferably it is the molecule of section (a) or (b) of this paragraph.

In particular, it was observed that in plants, especially in Arabidopsis thaliana, increasing or generating the activity of a gene product non-targeted with the activity of a “b1445-protein”, preferably being encoded by a gene comprising the nucleic acid sequence SEQ ID NO. 38289, preferably the coding region thereof, conferred the production of or the increase in citrulline compared with the wild type control.

The nucleic acid sequence of B1445 from Escherichia coli, e.g. as shown in the respective line in column 5 of Table I, application no. 3, has been published in B. And the activity of the gene product thereof is the activity of b1445-protein.

Accordingly, in one embodiment, the process of the present invention for producing glycine in a non-human organism, like a microorganism or a plant or a part thereof, comprises increasing or generating the activity of a gene product with the activity of a gene product conferring the activity of “b1445-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1445, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1445, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1445, or a functional equivalent or a homolog thereof as depicted in column 8 of Table II, application no. 3, preferably a homolog or functional equivalent as depicted in column 8 of Table II B, application no. 3, and being depicted in the same respective line as said B1445, and preferably the activity is increased non-targeted.

Accordingly, in one embodiment, the molecule which activity is to be increased in the process of the invention is the gene product with an activity as a “b1445-protein”, preferably it is the molecule of section (a) or (b) of this paragraph.

In particular, it was observed that in plants, especially in Arabidopsis thaliana, increasing or generating the activity of a gene product non-targeted with the activity of a “b1445-protein”, preferably being encoded by a gene comprising the nucleic acid sequence SEQ ID NO. 38289, preferably the coding region thereof, conferred the production of or the increase in glycine compared with the wild type control.

The nucleic acid sequence of B1445 from Escherichia coli, e.g. as shown in the respective line in column 5 of Table I, application no. 3, has been published in B. And the activity of the gene product thereof is the activity of b1445-protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “b1445-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1445, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1445, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1445, or a functional equivalent or a homolog thereof as depicted in column 8 of Table II, application no. 3, preferably a homolog or functional equivalent as depicted in column 8 of Table II B, application no. 3, and being depicted in the same respective line as said B1445, and preferably the activity is increased non-targeted.

Accordingly, in one embodiment, the molecule which activity is to be increased in the process of the invention is the gene product with an activity as a “b1445-protein”, preferably it is the molecule of section (a) or (b) of this paragraph.

In particular, it was observed that in plants, especially in Arabidopsis thaliana, increasing or generating the activity of a gene product non-targeted with the activity of a “b1445-protein”, preferably being encoded by a gene comprising the nucleic acid sequence SEQ ID NO. 38289, preferably the coding region thereof, conferred the production of or the increase in phenylalanine compared with the wild type control.

The nucleic acid sequence of B1445 from Escherichia coli, e.g. as shown in the respective line in column 5 of Table I, application no. 3, has been published in B. And the activity of the gene product thereof is the activity of b1445-protein.

Accordingly, in one embodiment, the process of the present invention for producing serine in a non-human organism, like a microorganism or a plant or a part thereof, comprises increasing or generating the activity of a gene product with the activity of a gene product conferring the activity of “b1445-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1445, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1445, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1445, or a functional equivalent or a homolog thereof as depicted in column 8 of Table II, application no. 3, preferably a homolog or functional equivalent as depicted in column 8 of Table II B, application no. 3, and being depicted in the same respective line as said B1445, and preferably the activity is increased non-targeted.

Accordingly, in one embodiment, the molecule which activity is to be increased in the process of the invention is the gene product with an activity as a “b1445-protein”, preferably it is the molecule of section (a) or (b) of this paragraph.

In particular, it was observed that in plants, especially in Arabidopsis thaliana, increasing or generating the activity of a gene product non-targeted with the activity of a “b1445-protein”, preferably being encoded by a gene comprising the nucleic acid sequence SEQ ID NO. 38289, preferably the coding region thereof, conferred the production of or the increase in serine compared with the wild type control.

The nucleic acid sequence of B1479 from Escherichia coli, e.g. as shown in the respective line in column 5 of Table I, application no. 3, has been published in B. And the activity of the gene product thereof is the activity of NADP-dependent malic enzyme.

Accordingly, in one embodiment, the process of the present invention for producing alanine in a non-human organism, like a microorganism or a plant or a part thereof, comprises increasing or generating the activity of a gene product with the activity of a gene product conferring the activity of “NADP-dependent malic enzyme”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1479, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1479, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1479, or a functional equivalent or a homolog thereof as depicted in column 8 of Table II, application no. 3, preferably a homolog or functional equivalent as depicted in column 8 of Table II B, application no. 3, and being depicted in the same respective line as said B1479, and preferably the activity is increased non-targeted.

Accordingly, in one embodiment, the molecule which activity is to be increased in the process of the invention is the gene product with an activity as a “NADP-dependent malic enzyme”, preferably it is the molecule of section (a) or (b) of this paragraph.

In particular, it was observed that in plants, especially in Arabidopsis thaliana, increasing or generating the activity of a gene product non-targeted with the activity of a “NADP-dependent malic enzyme”, preferably being encoded by a gene comprising the nucleic acid sequence SEQ ID NO. 77774, preferably the coding region thereof, conferred the production of or the increase in alanine compared with the wild type control.

The nucleic acid sequence of B1479 from Escherichia coli, e.g. as shown in the respective line in column 5 of Table I, application no. 3, has been published in B. And the activity of the gene product thereof is the activity of NADP-dependent malic enzyme.

Accordingly, in one embodiment, the process of the present invention for producing glycine in a non-human organism, like a microorganism or a plant or a part thereof, comprises increasing or generating the activity of a gene product with the activity of a gene product conferring the activity of “NADP-dependent malic enzyme”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1479, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1479, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1479, or a functional equivalent or a homolog thereof as depicted in column 8 of Table II, application no. 3, preferably a homolog or functional equivalent as depicted in column 8 of Table II B, application no. 3, and being depicted in the same respective line as said B1479, and preferably the activity is increased non-targeted.

Accordingly, in one embodiment, the molecule which activity is to be increased in the process of the invention is the gene product with an activity as a “NADP-dependent malic enzyme”, preferably it is the molecule of section (a) or (b) of this paragraph.

In particular, it was observed that in plants, especially in Arabidopsis thaliana, increasing or generating the activity of a gene product non-targeted with the activity of a “NADP-dependent malic enzyme”, preferably being encoded by a gene comprising the nucleic acid sequence SEQ ID NO. 77774, preferably the coding region thereof, conferred the production of or the increase in glycine compared with the wild type control.

The nucleic acid sequence of B1479 from Escherichia coli, e.g. as shown in the respective line in column 5 of Table I, application no. 3, has been published in B. And the activity of the gene product thereof is the activity of NADP-dependent malic enzyme.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “NADP-dependent malic enzyme”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1479, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1479, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1479, or a functional equivalent or a homolog thereof as depicted in column 8 of Table II, application no. 3, preferably a homolog or functional equivalent as depicted in column 8 of Table II B, application no. 3, and being depicted in the same respective line as said B1479, and preferably the activity is increased non-targeted.

Accordingly, in one embodiment, the molecule which activity is to be increased in the process of the invention is the gene product with an activity as a “NADP-dependent malic enzyme”, preferably it is the molecule of section (a) or (b) of this paragraph.

In particular, it was observed that in plants, especially in Arabidopsis thaliana, increasing or generating the activity of a gene product non-targeted with the activity of a “NADP-dependent malic enzyme”, preferably being encoded by a gene comprising the nucleic acid sequence SEQ ID NO. 77774, preferably the coding region thereof, conferred the production of or the increase in phenylalanine compared with the wild type control.

The nucleic acid sequence of B1479 from Escherichia coli, e.g. as shown in the respective line in column 5 of Table I, application no. 3, has been published in B. And the activity of the gene product thereof is the activity of NADP-dependent malic enzyme.

Accordingly, in one embodiment, the process of the present invention for producing serine in a non-human organism, like a microorganism or a plant or a part thereof, comprises increasing or generating the activity of a gene product with the activity of a gene product conferring the activity of “NADP-dependent malic enzyme”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1479, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1479, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1479, or a functional equivalent or a homolog thereof as depicted in column 8 of Table II, application no. 3, preferably a homolog or functional equivalent as depicted in column 8 of Table II B, application no. 3, and being depicted in the same respective line as said B1479, and preferably the activity is increased non-targeted.

Accordingly, in one embodiment, the molecule which activity is to be increased in the process of the invention is the gene product with an activity as a “NADP-dependent malic enzyme”, preferably it is the molecule of section (a) or (b) of this paragraph.

In particular, it was observed that in plants, especially in Arabidopsis thaliana, increasing or generating the activity of a gene product non-targeted with the activity of a “NADP-dependent malic enzyme”, preferably being encoded by a gene comprising the nucleic acid sequence SEQ ID NO. 77774, preferably the coding region thereof, conferred the production of or the increase in serine compared with the wild type control.

The nucleic acid sequence of B1479 from Escherichia coli, e.g. as shown in the respective line in column 5 of Table I, application no. 3, has been published in B. And the activity of the gene product thereof is the activity of NADP-dependent malic enzyme.

Accordingly, in one embodiment, the process of the present invention for producing tyrosine in a non-human organism, like a microorganism or a plant or a part thereof, comprises increasing or generating the activity of a gene product with the activity of a gene product conferring the activity of “NADP-dependent malic enzyme”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1479, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1479, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1479, or a functional equivalent or a homolog thereof as depicted in column 8 of Table II, application no. 3, preferably a homolog or functional equivalent as depicted in column 8 of Table II B, application no. 3, and being depicted in the same respective line as said B1479, and preferably the activity is increased non-targeted.

Accordingly, in one embodiment, the molecule which activity is to be increased in the process of the invention is the gene product with an activity as a “NADP-dependent malic enzyme”, preferably it is the molecule of section (a) or (b) of this paragraph.

In particular, it was observed that in plants, especially in Arabidopsis thaliana, increasing or generating the activity of a gene product non-targeted with the activity of a “NADP-dependent malic enzyme”, preferably being encoded by a gene comprising the nucleic acid sequence SEQ ID NO. 77774, preferably the coding region thereof, conferred the production of or the increase in tyrosine compared with the wild type control.

The nucleic acid sequence of B1522 from Escherichia coli, e.g. as shown in the respective line in column 5 of Table I, application no. 3, has been published in B. And the activity of the gene product thereof is the activity of b1522-protein.

Accordingly, in one embodiment, the process of the present invention for producing asparagine in a non-human organism, like a microorganism or a plant or a part thereof, comprises increasing or generating the activity of a gene product with the activity of a gene product conferring the activity of “b1522-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1522, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1522, or a functional equivalent or a homolog thereof as depicted in column 8 of Table II, application no. 3, preferably a homolog or functional equivalent as depicted in column 8 of Table II B, application no. 3, and being depicted in the same respective line as said B1522, and preferably the activity is increased non-targeted.

Accordingly, in one embodiment, the molecule which activity is to be increased in the process of the invention is the gene product with an activity as a “b1522-protein”, preferably it is the molecule of section (a) or (b) of this paragraph.

In particular, it was observed that in plants, especially in Arabidopsis thaliana, increasing or generating the activity of a gene product non-targeted with the activity of a “b1522-protein”, preferably being encoded by a gene comprising the nucleic acid sequence SEQ ID NO. 7947, preferably the coding region thereof, conferred the production of or the increase in asparagine compared with the wild type control.

The nucleic acid sequence of B1522 from Escherichia coli, e.g. as shown in the respective line in column 5 of Table I, application no. 3, has been published in B. And the activity of the gene product thereof is the activity of b1522-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “b1522-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1522, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1522, or a functional equivalent or a homolog thereof as depicted in column 8 of Table II, application no. 3, preferably a homolog or functional equivalent as depicted in column 8 of Table II B, application no. 3, and being depicted in the same respective line as said B1522, and preferably the activity is increased non-targeted.

Accordingly, in one embodiment, the molecule which activity is to be increased in the process of the invention is the gene product with an activity as a “b1522-protein”, preferably it is the molecule of section (a) or (b) of this paragraph.

In particular, it was observed that in plants, especially in Arabidopsis thaliana, increasing or generating the activity of a gene product non-targeted with the activity of a “b1522-protein”, preferably being encoded by a gene comprising the nucleic acid sequence SEQ ID NO. 7947, preferably the coding region thereof, conferred the production of or the increase in aspartate compared with the wild type control.

The nucleic acid sequence of B1522 from Escherichia coli, e.g. as shown in the respective line in column 5 of Table I, application no. 3, has been published in B. And the activity of the gene product thereof is the activity of b1522-protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “b1522-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1522, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1522, or a functional equivalent or a homolog thereof as depicted in column 8 of Table II, application no. 3, preferably a homolog or functional equivalent as depicted in column 8 of Table II B, application no. 3, and being depicted in the same respective line as said B1522, and preferably the activity is increased non-targeted.

Accordingly, in one embodiment, the molecule which activity is to be increased in the process of the invention is the gene product with an activity as a “b1522-protein”, preferably it is the molecule of section (a) or (b) of this paragraph.

In particular, it was observed that in plants, especially in Arabidopsis thaliana, increasing or generating the activity of a gene product non-targeted with the activity of a “b1522-protein”, preferably being encoded by a gene comprising the nucleic acid sequence SEQ ID NO. 7947, preferably the coding region thereof, conferred the production of or the increase in phenylalanine compared with the wild type control.

The nucleic acid sequence of B1522 from Escherichia coli, e.g. as shown in the respective line in column 5 of Table I, application no. 3, has been published in B. And the activity of the gene product thereof is the activity of b1522-protein.

Accordingly, in one embodiment, the process of the present invention for producing serine in a non-human organism, like a microorganism or a plant or a part thereof, comprises increasing or generating the activity of a gene product with the activity of a gene product conferring the activity of “b1522-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1522, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1522, or a functional equivalent or a homolog thereof as depicted in column 8 of Table II, application no. 3, preferably a homolog or functional equivalent as depicted in column 8 of Table II B, application no. 3, and being depicted in the same respective line as said B1522, and preferably the activity is increased non-targeted.

Accordingly, in one embodiment, the molecule which activity is to be increased in the process of the invention is the gene product with an activity as a “b1522-protein”, preferably it is the molecule of section (a) or (b) of this paragraph.

In particular, it was observed that in plants, especially in Arabidopsis thaliana, increasing or generating the activity of a gene product non-targeted with the activity of a “b1522-protein”, preferably being encoded by a gene comprising the nucleic acid sequence SEQ ID NO. 7947, preferably the coding region thereof, conferred the production of or the increase in serine compared with the wild type control.

The nucleic acid sequence of B1522 from Escherichia coli, e.g. as shown in the respective line in column 5 of Table I, application no. 3, has been published in B. And the activity of the gene product thereof is the activity of b1522-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “b1522-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1522, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1522, or a functional equivalent or a homolog thereof as depicted in column 8 of Table II, application no. 3, preferably a homolog or functional equivalent as depicted in column 8 of Table II B, application no. 3, and being depicted in the same respective line as said B1522, and preferably the activity is increased non-targeted.

Accordingly, in one embodiment, the molecule which activity is to be increased in the process of the invention is the gene product with an activity as a “b1522-protein”, preferably it is the molecule of section (a) or (b) of this paragraph.

In particular, it was observed that in plants, especially in Arabidopsis thaliana, increasing or generating the activity of a gene product non-targeted with the activity of a “b1522-protein”, preferably being encoded by a gene comprising the nucleic acid sequence SEQ ID NO. 7947, preferably the coding region thereof, conferred the production of or the increase in tyrosine compared with the wild type control.

The nucleic acid sequence of B1522 from Escherichia coli, e.g. as shown in the respective line in column 5 of Table I, application no. 3, has been published in B. And the activity of the gene product thereof is the activity of b1522-protein.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “b1522-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1522, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1522, or a functional equivalent or a homolog thereof as depicted in column 8 of Table II, application no. 3, preferably a homolog or functional equivalent as depicted in column 8 of Table II B, application no. 3, and being depicted in the same respective line as said B1522, and preferably the activity is increased non-targeted.

Accordingly, in one embodiment, the molecule which activity is to be increased in the process of the invention is the gene product with an activity as a “b1522-protein”, preferably it is the molecule of section (a) or (b) of this paragraph.

In particular, it was observed that in plants, especially in Arabidopsis thaliana, increasing or generating the activity of a gene product non-targeted with the activity of a “b1522-protein”, preferably being encoded by a gene comprising the nucleic acid sequence SEQ ID NO. 7947, preferably the coding region thereof, conferred the production of or the increase in 5-oxoproline compared with the wild type control.

The nucleic acid sequence of B1583 from Escherichia coli, e.g. as shown in the respective line in column 5 of Table I, application no. 3, has been published in B. And the activity of the gene product thereof is the activity of b1583-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “b1583-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1583, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1583, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1583, or a functional equivalent or a homolog thereof as depicted in column 8 of Table II, application no. 3, preferably a homolog or functional equivalent as depicted in column 8 of Table II B, application no. 3, and being depicted in the same respective line as said B1583, and preferably the activity is increased non-targeted.

Accordingly, in one embodiment, the molecule which activity is to be increased in the process of the invention is the gene product with an activity as a “b1583-protein”, preferably it is the molecule of section (a) or (b) of this paragraph.

In particular, it was observed that in plants, especially in Arabidopsis thaliana, increasing or generating the activity of a gene product non-targeted with the activity of a “b1583-protein”, preferably being encoded by a gene comprising the nucleic acid sequence SEQ ID NO. 78138, preferably the coding region thereof, conferred the production of or the increase in tyrosine compared with the wild type control.

The nucleic acid sequence of B1589 from Escherichia coli, e.g. as shown in the respective line in column 5 of Table I, application no. 3, has been published in B. And the activity of the gene product thereof is the activity of Fe—S subunit of oxidoreductase.

Accordingly, in one embodiment, the process of the present invention for producing aspartate in a non-human organism, like a microorganism or a plant or a part thereof, comprises increasing or generating the activity of a gene product with the activity of a gene product conferring the activity of “Fe—S subunit of oxidoreductase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1589, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1589, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1589, or a functional equivalent or a homolog thereof as depicted in column 8 of Table II, application no. 3, preferably a homolog or functional equivalent as depicted in column 8 of Table II B, application no. 3, and being depicted in the same respective line as said B1589, and preferably the activity is increased non-targeted.

Accordingly, in one embodiment, the molecule which activity is to be increased in the process of the invention is the gene product with an activity as a “Fe—S subunit of oxidoreductase”, preferably it is the molecule of section (a) or (b) of this paragraph.

In particular, it was observed that in plants, especially in Arabidopsis thaliana, increasing or generating the activity of a gene product non-targeted with the activity of a “Fe—S subunit of oxidoreductase”, preferably being encoded by a gene comprising the nucleic acid sequence SEQ ID NO. 78154, preferably the coding region thereof, conferred the production of or the increase in aspartate compared with the wild type control.

The nucleic acid sequence of B1597 from Escherichia coli, e.g. as shown in the respective line in column 5 of Table I, application no. 3, has been published in B. And the activity of the gene product thereof is the activity of acid shock protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “acid shock protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1597, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1597, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1597, or a functional equivalent or a homolog thereof as depicted in column 8 of Table II, application no. 3, preferably a homolog or functional equivalent as depicted in column 8 of Table II B, application no. 3, and being depicted in the same respective line as said B1597, and preferably the activity is increased non-targeted.

Accordingly, in one embodiment, the molecule which activity is to be increased in the process of the invention is the gene product with an activity as a “acid shock protein”, preferably it is the molecule of section (a) or (b) of this paragraph.

In particular, it was observed that in plants, especially in Arabidopsis thaliana, increasing or generating the activity of a gene product non-targeted with the activity of a “acid shock protein”, preferably being encoded by a gene comprising the nucleic acid sequence SEQ ID NO. 38300, preferably the coding region thereof, conferred the production of or the increase in phenylalanine compared with the wild type control.

The nucleic acid sequence of B1597 from Escherichia coli, e.g. as shown in the respective line in column 5 of Table I, application no. 3, has been published in B. And the activity of the gene product thereof is the activity of acid shock protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “acid shock protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1597, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1597, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1597, or a functional equivalent or a homolog thereof as depicted in column 8 of Table II, application no. 3, preferably a homolog or functional equivalent as depicted in column 8 of Table II B, application no. 3, and being depicted in the same respective line as said B1597, and preferably the activity is increased non-targeted.

Accordingly, in one embodiment, the molecule which activity is to be increased in the process of the invention is the gene product with an activity as a “acid shock protein”, preferably it is the molecule of section (a) or (b) of this paragraph.

In particular, it was observed that in plants, especially in Arabidopsis thaliana, increasing or generating the activity of a gene product non-targeted with the activity of a “acid shock protein”, preferably being encoded by a gene comprising the nucleic acid sequence SEQ ID NO. 38300, preferably the coding region thereof, conferred the production of or the increase in tyrosine compared with the wild type control.

The nucleic acid sequence of B1651 from Escherichia coli, e.g. as shown in the respective line in column 5 of Table I, application no. 3, has been published in B. And the activity of the gene product thereof is the activity of methylglyoxalase.

Accordingly, in one embodiment, the process of the present invention for producing alanine in a non-human organism, like a microorganism or a plant or a part thereof, comprises increasing or generating the activity of a gene product with the activity of a gene product conferring the activity of “methylglyoxalase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1651, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1651, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1651, or a functional equivalent or a homolog thereof as depicted in column 8 of Table II, application no. 3, preferably a homolog or functional equivalent as depicted in column 8 of Table II B, application no. 3, and being depicted in the same respective line as said B1651, and preferably the activity is increased non-targeted.

Accordingly, in one embodiment, the molecule which activity is to be increased in the process of the invention is the gene product with an activity as a “methylglyoxalase”, preferably it is the molecule of section (a) or (b) of this paragraph.

In particular, it was observed that in plants, especially in Arabidopsis thaliana, increasing or generating the activity of a gene product non-targeted with the activity of a “methylglyoxalase”, preferably being encoded by a gene comprising the nucleic acid sequence SEQ ID NO. 78265, preferably the coding region thereof, conferred the production of or the increase in alanine compared with the wild type control.

The nucleic acid sequence of B1670 from Escherichia coli, e.g. as shown in the respective line in column 5 of Table I, application no. 3, has been published in B. And the activity of the gene product thereof is the activity of b1670-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “b1670-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1670, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1670, or a functional equivalent or a homolog thereof as depicted in column 8 of Table II, application no. 3, preferably a homolog or functional equivalent as depicted in column 8 of Table II B, application no. 3, and being depicted in the same respective line as said B1670, and preferably the activity is increased non-targeted.

Accordingly, in one embodiment, the molecule which activity is to be increased in the process of the invention is the gene product with an activity as a “b1670-protein”, preferably it is the molecule of section (a) or (b) of this paragraph.

In particular, it was observed that in plants, especially in Arabidopsis thaliana, increasing or generating the activity of a gene product non-targeted with the activity of a “b1670-protein”, preferably being encoded by a gene comprising the nucleic acid sequence SEQ ID NO. 78753, preferably the coding region thereof, conferred the production of or the increase in aspartate compared with the wild type control.

The nucleic acid sequence of B1672 from Escherichia coli, e.g. as shown in the respective line in column 5 of Table I, application no. 3, has been published in B. And the activity of the gene product thereof is the activity of b1672-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “b1672-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1672, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1672, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1672, or a functional equivalent or a homolog thereof as depicted in column 8 of Table II, application no. 3, preferably a homolog or functional equivalent as depicted in column 8 of Table II B, application no. 3, and being depicted in the same respective line as said B1672, and preferably the activity is increased non-targeted.

Accordingly, in one embodiment, the molecule which activity is to be increased in the process of the invention is the gene product with an activity as a “b1672-protein”, preferably it is the molecule of section (a) or (b) of this paragraph.

In particular, it was observed that in plants, especially in Arabidopsis thaliana, increasing or generating the activity of a gene product non-targeted with the activity of a “b1672-protein”, preferably being encoded by a gene comprising the nucleic acid sequence SEQ ID NO. 78771, preferably the coding region thereof, conferred the production of or the increase in aspartate compared with the wild type control.

The nucleic acid sequence of B1747 from Escherichia coli, e.g. as shown in the respective line in column 5 of Table I, application no. 3, has been published in B. And the activity of the gene product thereof is the activity of arginine N-succinyltransferase.

Accordingly, in one embodiment, the process of the present invention for producing alanine in a non-human organism, like a microorganism or a plant or a part thereof, comprises increasing or generating the activity of a gene product with the activity of a gene product conferring the activity of “arginine N-succinyltransferase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1747, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1747, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1747, or a functional equivalent or a homolog thereof as depicted in column 8 of Table II, application no. 3, preferably a homolog or functional equivalent as depicted in column 8 of Table II B, application no. 3, and being depicted in the same respective line as said B1747, and preferably the activity is increased plastidic.

Accordingly, in one embodiment, the molecule which activity is to be increased in the process of the invention is the gene product with an activity as a “arginine N-succinyltransferase”, preferably it is the molecule of section (a) or (b) of this paragraph.

In particular, it was observed that in plants, especially in Arabidopsis thaliana, increasing or generating the activity of a gene product plastidic with the activity of a “arginine N-succinyltransferase”, preferably being encoded by a gene comprising the nucleic acid sequence SEQ ID NO. 78779, preferably the coding region thereof, conferred the production of or the increase in alanine compared with the wild type control.

The nucleic acid sequence of B1755 from Escherichia coli, e.g. as shown in the respective line in column 5 of Table I, application no. 3, has been published in B. And the activity of the gene product thereof is the activity of ABC transporter permease protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “ABC transporter permease protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1755, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1755, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1755, or a functional equivalent or a homolog thereof as depicted in column 8 of Table II, application no. 3, preferably a homolog or functional equivalent as depicted in column 8 of Table II B, application no. 3, and being depicted in the same respective line as said B1755, and preferably the activity is increased non-targeted.

Accordingly, in one embodiment, the molecule which activity is to be increased in the process of the invention is the gene product with an activity as a “ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “ABC transporter permease protein”, preferably being encoded by a gene comprising the nucleic acid sequence SEQ ID NO. 78852, preferably the coding region thereof, conferred the production of or the increase in aspartate compared with the wild type control.

The nucleic acid sequence of B1755 from Escherichia coli, e.g. as shown in the respective line in column 5 of Table I, application no. 3, has been published in B. And the activity of the gene product thereof is the activity of ABC transporter permease protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “ABC transporter permease protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1755, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1755, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1755, or a functional equivalent or a homolog thereof as depicted in column 8 of Table II, application no. 3, preferably a homolog or functional equivalent as depicted in column 8 of Table II B, application no. 3, and being depicted in the same respective line as said B1755, and preferably the activity is increased non-targeted.

Accordingly, in one embodiment, the molecule which activity is to be increased in the process of the invention is the gene product with an activity as a “ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “ABC transporter permease protein”, preferably being encoded by a gene comprising the nucleic acid sequence SEQ ID NO. 78852, preferably the coding region thereof, conferred the production of or the increase in tyrosine compared with the wild type control.

The nucleic acid sequence of B1791 from Escherichia coli, e.g. as shown in the respective line in column 5 of Table I, application no. 3, has been published in B. And the activity of the gene product thereof is the activity of transport protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “transport protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1791, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1791, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1791, or a functional equivalent or a homolog thereof as depicted in column 8 of Table II, application no. 3, preferably a homolog or functional equivalent as depicted in column 8 of Table II B, application no. 3, and being depicted in the same respective line as said B1791, and preferably the activity is increased non-targeted.

Accordingly, in one embodiment, the molecule which activity is to be increased in the process of the invention is the gene product with an activity as a “transport protein”, preferably it is the molecule of section (a) or (b) of this paragraph.

In particular, it was observed that in plants, especially in Arabidopsis thaliana, increasing or generating the activity of a gene product non-targeted with the activity of a “transport protein”, preferably being encoded by a gene comprising the nucleic acid sequence SEQ ID NO. 78883, preferably the coding region thereof, conferred the production of or the increase in aspartate compared with the wild type control.

The nucleic acid sequence of B1837 from Escherichia coli, e.g. as shown in the respective line in column 5 of Table I, application no. 3, has been published in B. And the activity of the gene product thereof is the activity of b1837-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “b1837-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1837, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1837, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1837, or a functional equivalent or a homolog thereof as depicted in column 8 of Table II, application no. 3, preferably a homolog or functional equivalent as depicted in column 8 of Table II B, application no. 3, and being depicted in the same respective line as said B1837, and preferably the activity is increased non-targeted.

Accordingly, in one embodiment, the molecule which activity is to be increased in the process of the invention is the gene product with an activity as a “b1837-protein”, preferably it is the molecule of section (a) or (b) of this paragraph.

In particular, it was observed that in plants, especially in Arabidopsis thaliana, increasing or generating the activity of a gene product non-targeted with the activity of a “b1837-protein”, preferably being encoded by a gene comprising the nucleic acid sequence SEQ ID NO. 78953, preferably the coding region thereof, conferred the production of or the increase in aspartate compared with the wild type control.

The nucleic acid sequence of B1838 from Escherichia coli, e.g. as shown in the respective line in column 5 of Table I, application no. 3, has been published in B. And the activity of the gene product thereof is the activity of serine/threonine-protein phosphatase.

Accordingly, in one embodiment, the process of the present invention for producing aspartate in a non-human organism, like a microorganism or a plant or a part thereof, comprises increasing or generating the activity of a gene product with the activity of a gene product conferring the activity of “serine/threonine-protein phosphatase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1838, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1838, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1838, or a functional equivalent or a homolog thereof as depicted in column 8 of Table II, application no. 3, preferably a homolog or functional equivalent as depicted in column 8 of Table II B, application no. 3, and being depicted in the same respective line as said B1838, and preferably the activity is increased non-targeted.

Accordingly, in one embodiment, the molecule which activity is to be increased in the process of the invention is the gene product with an activity as a “serine/threonine-protein phosphatase”, preferably it is the molecule of section (a) or (b) of this paragraph.

In particular, it was observed that in plants, especially in Arabidopsis thaliana, increasing or generating the activity of a gene product non-targeted with the activity of a “serine/threonineprotein phosphatase”, preferably being encoded by a gene comprising the nucleic acid sequence SEQ ID NO. 8316, preferably the coding region thereof, conferred the production of or the increase in aspartate compared with the wild type control.

The nucleic acid sequence of B1845 from Escherichia coli, e.g. as shown in the respective line in column 5 of Table I, application no. 3, has been published in B. And the activity of the gene product thereof is the activity of protease.

Accordingly, in one embodiment, the process of the present invention for producing aspartate in a non-human organism, like a microorganism or a plant or a part thereof, comprises increasing or generating the activity of a gene product with the activity of a gene product conferring the activity of “protease”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1845, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1845, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1845, or a functional equivalent or a homolog thereof as depicted in column 8 of Table II, application no. 3, preferably a homolog or functional equivalent as depicted in column 8 of Table II B, application no. 3, and being depicted in the same respective line as said B1845, and preferably the activity is increased non-targeted.

Accordingly, in one embodiment, the molecule which activity is to be increased in the process of the invention is the gene product with an activity as a “protease”, preferably it is the molecule of section (a) or (b) of this paragraph.

In particular, it was observed that in plants, especially in Arabidopsis thaliana, increasing or generating the activity of a gene product non-targeted with the activity of a “protease”, preferably being encoded by a gene comprising the nucleic acid sequence SEQ ID NO. 38573, preferably the coding region thereof, conferred the production of or the increase in aspartate compared with the wild type control.

The nucleic acid sequence of B1845 from Escherichia coli, e.g. as shown in the respective line in column 5 of Table I, application no. 3, has been published in B. And the activity of the gene product thereof is the activity of protease.

Accordingly, in one embodiment, the process of the present invention for producing serine in a non-human organism, like a microorganism or a plant or a part thereof, comprises increasing or generating the activity of a gene product with the activity of a gene product conferring the activity of “protease”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1845, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1845, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1845, or a functional equivalent or a homolog thereof as depicted in column 8 of Table II, application no. 3, preferably a homolog or functional equivalent as depicted in column 8 of Table II B, application no. 3, and being depicted in the same respective line as said B1845, and preferably the activity is increased non-targeted.

Accordingly, in one embodiment, the molecule which activity is to be increased in the process of the invention is the gene product with an activity as a “protease”, preferably it is the molecule of section (a) or (b) of this paragraph.

In particular, it was observed that in plants, especially in Arabidopsis thaliana, increasing or generating the activity of a gene product non-targeted with the activity of a “protease”, preferably being encoded by a gene comprising the nucleic acid sequence SEQ ID NO. 38573, preferably the coding region thereof, conferred the production of or the increase in serine compared with the wild type control.

The nucleic acid sequence of B1854 from Escherichia coli, e.g. as shown in the respective line in column 5 of Table I, application no. 3, has been published in B. And the activity of the gene product thereof is the activity of pyruvate kinase.

Accordingly, in one embodiment, the process of the present invention for producing asparagine in a non-human organism, like a microorganism or a plant or a part thereof, comprises increasing or generating the activity of a gene product with the activity of a gene product conferring the activity of “pyruvate kinase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1854, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1854, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1854, or a functional equivalent or a homolog thereof as depicted in column 8 of Table II, application no. 3, preferably a homolog or functional equivalent as depicted in column 8 of Table II B, application no. 3, and being depicted in the same respective line as said B1854, and preferably the activity is increased plastidic.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 8363, preferably the coding region thereof, conferred the production of or the increase in asparagine compared with the wild type control.

The nucleic acid sequence of B1873 from Escherichia coli, e.g. as shown in the respective line in column 5 of Table I, application no. 3, has been published in B. And the activity of the gene product thereof is the activity of cytochrome c-type protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “cytochrome c-type protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1873, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1873, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1873, or a functional equivalent or a homolog thereof as depicted in column 8 of Table II, application no. 3, preferably a homolog or functional equivalent as depicted in column 8 of Table II B, application no. 3, and being depicted in the same respective line as said B1873, and preferably the activity is increased non-targeted.

Accordingly, in one embodiment, the molecule which activity is to be increased in the process of the invention is the gene product with an activity as a “cytochrome c-type protein”, preferably it is the molecule of section (a) or (b) of this paragraph.

In particular, it was observed that in plants, especially in Arabidopsis thaliana, increasing or generating the activity of a gene product non-targeted with the activity of a “cytochrome c-type protein”, preferably being encoded by a gene comprising the nucleic acid sequence SEQ ID NO. 78970, preferably the coding region thereof, conferred the production of or the increase in tyrosine compared with the wild type control.

The nucleic acid sequence of B1897 from Escherichia coli, e.g. as shown in the respective line in column 5 of Table I, application no. 3, has been published in B. And the activity of the gene product thereof is the activity of trehalose-phosphatase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “trehalose-phosphatase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1897, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1897, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1897, or a functional equivalent or a homolog thereof as depicted in column 8 of Table II, application no. 3, preferably a homolog or functional equivalent as depicted in column 8 of Table II B, application no. 3, and being depicted in the same respective line as said B1897, and preferably the activity is increased non-targeted.

Accordingly, in one embodiment, the molecule which activity is to be increased in the process of the invention is the gene product with an activity as a “trehalose-phosphatase”, preferably it is the molecule of section (a) or (b) of this paragraph.

In particular, it was observed that in plants, especially in Arabidopsis thaliana, increasing or generating the activity of a gene product non-targeted with the activity of a “trehalose-phosphatase”, preferably being encoded by a gene comprising the nucleic acid sequence SEQ ID NO. 79056, preferably the coding region thereof, conferred the production of or the increase in phenylalanine compared with the wild type control.

The nucleic acid sequence of B1897 from Escherichia coli, e.g. as shown in the respective line in column 5 of Table I, application no. 3, has been published in B. And the activity of the gene product thereof is the activity of trehalose-phosphatase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “trehalose-phosphatase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1897, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1897, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1897, or a functional equivalent or a homolog thereof as depicted in column 8 of Table II, application no. 3, preferably a homolog or functional equivalent as depicted in column 8 of Table II B, application no. 3, and being depicted in the same respective line as said B1897, and preferably the activity is increased non-targeted.

Accordingly, in one embodiment, the molecule which activity is to be increased in the process of the invention is the gene product with an activity as a “trehalose-phosphatase”, preferably it is the molecule of section (a) or (b) of this paragraph.

In particular, it was observed that in plants, especially in Arabidopsis thaliana, increasing or generating the activity of a gene product non-targeted with the activity of a “trehalosephosphatase”, preferably being encoded by a gene comprising the nucleic acid sequence SEQ ID NO. 79056, preferably the coding region thereof, conferred the production of or the increase in tyrosine compared with the wild type control.

The nucleic acid sequence of B1898 from Escherichia coli, e.g. as shown in the respective line in column 5 of Table I, application no. 3, has been published in B. And the activity of the gene product thereof is the activity of b1898-protein.

Accordingly, in one embodiment, the process of the present invention for producing serine in a non-human organism, like a microorganism or a plant or a part thereof, comprises increasing or generating the activity of a gene product with the activity of a gene product conferring the activity of “b1898-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1898, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1898, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1898, or a functional equivalent or a homolog thereof as depicted in column 8 of Table II, application no. 3, preferably a homolog or functional equivalent as depicted in column 8 of Table II B, application no. 3, and being depicted in the same respective line as said B1898, and preferably the activity is increased non-targeted.

Accordingly, in one embodiment, the molecule which activity is to be increased in the process of the invention is the gene product with an activity as a “b1898-protein”, preferably it is the molecule of section (a) or (b) of this paragraph.

In particular, it was observed that in plants, especially in Arabidopsis thaliana, increasing or generating the activity of a gene product non-targeted with the activity of a “b1898-protein”, preferably being encoded by a gene comprising the nucleic acid sequence SEQ ID NO. 38767, preferably the coding region thereof, conferred the production of or the increase in serine compared with the wild type control.

The nucleic acid sequence of B1930 from Escherichia coli, e.g. as shown in the respective line in column 5 of Table I, application no. 3, has been published in B. And the activity of the gene product thereof is the activity of b1930-protein.

Accordingly, in one embodiment, the process of the present invention for producing serine in a non-human organism, like a microorganism or a plant or a part thereof, comprises increasing or generating the activity of a gene product with the activity of a gene product conferring the activity of “b1930-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1930, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1930, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1930, or a functional equivalent or a homolog thereof as depicted in column 8 of Table II, application no. 3, preferably a homolog or functional equivalent as depicted in column 8 of Table II B, application no. 3, and being depicted in the same respective line as said B1930, and preferably the activity is increased non-targeted.

Accordingly, in one embodiment, the molecule which activity is to be increased in the process of the invention is the gene product with an activity as a “b1930-protein”, preferably it is the molecule of section (a) or (b) of this paragraph.

In particular, it was observed that in plants, especially in Arabidopsis thaliana, increasing or generating the activity of a gene product non-targeted with the activity of a “b1930-protein”, preferably being encoded by a gene comprising the nucleic acid sequence SEQ ID NO. 79181, preferably the coding region thereof, conferred the production of or the increase in serine compared with the wild type control.

The nucleic acid sequence of B1981 from Escherichia coli, e.g. as shown in the respective line in column 5 of Table I, application no. 3, has been published in B. And the activity of the gene product thereof is the activity of transport protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “transport protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B1981, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B1981, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B1981, or a functional equivalent or a homolog thereof as depicted in column 8 of Table II, application no. 3, preferably a homolog or functional equivalent as depicted in column 8 of Table II B, application no. 3, and being depicted in the same respective line as said B1981, and preferably the activity is increased non-targeted.

Accordingly, in one embodiment, the molecule which activity is to be increased in the process of the invention is the gene product with an activity as a “transport protein”, preferably it is the molecule of section (a) or (b) of this paragraph.

In particular, it was observed that in plants, especially in Arabidopsis thaliana, increasing or generating the activity of a gene product non-targeted with the activity of a “transport protein”, preferably being encoded by a gene comprising the nucleic acid sequence SEQ ID NO. 38899, preferably the coding region thereof, conferred the production of or the increase in aspartate compared with the wild type control.

The nucleic acid sequence of B2027 from Escherichia coli, e.g. as shown in the respective line in column 5 of Table I, application no. 3, has been published in B. And the activity of the gene product thereof is the activity of O-antigen chain length determinant.

Accordingly, in one embodiment, the process of the present invention for producing tyrosine in a non-human organism, like a microorganism or a plant or a part thereof, comprises increasing or generating the activity of a gene product with the activity of a gene product conferring the activity of “O-antigen chain length determinant”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2027, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2027, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2027, or a functional equivalent or a homolog thereof as depicted in column 8 of Table II, application no. 3, preferably a homolog or functional equivalent as depicted in column 8 of Table II B, application no. 3, and being depicted in the same respective line as said B2027, and preferably the activity is increased non-targeted.

Accordingly, in one embodiment, the molecule which activity is to be increased in the process of the invention is the gene product with an activity as a “O-antigen chain length determinant”, preferably it is the molecule of section (a) or (b) of this paragraph.

In particular, it was observed that in plants, especially in Arabidopsis thaliana, increasing or generating the activity of a gene product non-targeted with the activity of a “O-antigen chain length determinant”, preferably being encoded by a gene comprising the nucleic acid sequence SEQ ID NO. 79217, preferably the coding region thereof, conferred the production of or the increase in tyrosine compared with the wild type control.

The nucleic acid sequence of B2032 from Escherichia coli, e.g. as shown in the respective line in column 5 of Table I, application no. 3, has been published in B. And the activity of the gene product thereof is the activity of b2032-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “b2032-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2032, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2032, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2032, or a functional equivalent or a homolog thereof as depicted in column 8 of Table II, application no. 3, preferably a homolog or functional equivalent as depicted in column 8 of Table II B, application no. 3, and being depicted in the same respective line as said B2032, and preferably the activity is increased non-targeted.

Accordingly, in one embodiment, the molecule which activity is to be increased in the process of the invention is the gene product with an activity as a “b2032-protein”, preferably it is the molecule of section (a) or (b) of this paragraph.

In particular, it was observed that in plants, especially in Arabidopsis thaliana, increasing or generating the activity of a gene product non-targeted with the activity of a “b2032-protein”, preferably being encoded by a gene comprising the nucleic acid sequence SEQ ID NO. 8920, preferably the coding region thereof, conferred the production of or the increase in aspartate compared with the wild type control.

The nucleic acid sequence of B2063 from Escherichia coli, e.g. as shown in the respective line in column 5 of Table I, application no. 3, has been published in B. And the activity of the gene product thereof is the activity of transport protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “transport protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2063, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2063, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2063, or a functional equivalent or a homolog thereof as depicted in column 8 of Table II, application no. 3, preferably a homolog or functional equivalent as depicted in column 8 of Table II B, application no. 3, and being depicted in the same respective line as said B2063, and preferably the activity is increased non-targeted.

Accordingly, in one embodiment, the molecule which activity is to be increased in the process of the invention is the gene product with an activity as a “transport protein”, preferably it is the molecule of section (a) or (b) of this paragraph.

In particular, it was observed that in plants, especially in Arabidopsis thaliana, increasing or generating the activity of a gene product non-targeted with the activity of a “transport protein”, preferably being encoded by a gene comprising the nucleic acid sequence SEQ ID NO. 38947, preferably the coding region thereof, conferred the production of or the increase in aspartate compared with the wild type control.

The nucleic acid sequence of B2063 from Escherichia coli, e.g. as shown in the respective line in column 5 of Table I, application no. 3, has been published in B. And the activity of the gene product thereof is the activity of transport protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “transport protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2063, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2063, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2063, or a functional equivalent or a homolog thereof as depicted in column 8 of Table II, application no. 3, preferably a homolog or functional equivalent as depicted in column 8 of Table II B, application no. 3, and being depicted in the same respective line as said B2063, and preferably the activity is increased non-targeted.

Accordingly, in one embodiment, the molecule which activity is to be increased in the process of the invention is the gene product with an activity as a “transport protein”, preferably it is the molecule of section (a) or (b) of this paragraph.

In particular, it was observed that in plants, especially in Arabidopsis thaliana, increasing or generating the activity of a gene product non-targeted with the activity of a “transport protein”, preferably being encoded by a gene comprising the nucleic acid sequence SEQ ID NO. 38947, preferably the coding region thereof, conferred the production of or the increase in phenylalanine compared with the wild type control.

The nucleic acid sequence of B2063 from Escherichia coli, e.g. as shown in the respective line in column 5 of Table I, application no. 3, has been published in B. And the activity of the gene product thereof is the activity of transport protein.

Accordingly, in one embodiment, the process of the present invention for producing serine in a non-human organism, like a microorganism or a plant or a part thereof, comprises increasing or generating the activity of a gene product with the activity of a gene product conferring the activity of “transport protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2063, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2063, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2063, or a functional equivalent or a homolog thereof as depicted in column 8 of Table II, application no. 3, preferably a homolog or functional equivalent as depicted in column 8 of Table II B, application no. 3, and being depicted in the same respective line as said B2063, and preferably the activity is increased non-targeted.

Accordingly, in one embodiment, the molecule which activity is to be increased in the process of the invention is the gene product with an activity as a “transport protein”, preferably it is the molecule of section (a) or (b) of this paragraph.

In particular, it was observed that in plants, especially in Arabidopsis thaliana, increasing or generating the activity of a gene product non-targeted with the activity of a “transport protein”, preferably being encoded by a gene comprising the nucleic acid sequence SEQ ID NO. 38947, preferably the coding region thereof, conferred the production of or the increase in serine compared with the wild type control.

The nucleic acid sequence of B2063 from Escherichia coli, e.g. as shown in the respective line in column 5 of Table I, application no. 3, has been published in B. And the activity of the gene product thereof is the activity of transport protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “transport protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2063, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2063, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2063, or a functional equivalent or a homolog thereof as depicted in column 8 of Table II, application no. 3, preferably a homolog or functional equivalent as depicted in column 8 of Table II B, application no. 3, and being depicted in the same respective line as said B2063, and preferably the activity is increased non-targeted.

Accordingly, in one embodiment, the molecule which activity is to be increased in the process of the invention is the gene product with an activity as a “transport protein”, preferably it is the molecule of section (a) or (b) of this paragraph.

In particular, it was observed that in plants, especially in Arabidopsis thaliana, increasing or generating the activity of a gene product non-targeted with the activity of a “transport protein”, preferably being encoded by a gene comprising the nucleic acid sequence SEQ ID NO. 38947, preferably the coding region thereof, conferred the production of or the increase in tyrosine compared with the wild type control.

The nucleic acid sequence of B2066 from Escherichia coli, e.g. as shown in the respective line in column 5 of Table I, application no. 3, has been published in B. And the activity of the gene product thereof is the activity of uridine/cytidine kinase.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “uridine/cytidine kinase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2066, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2066, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2066, or a functional equivalent or a homolog thereof as depicted in column 8 of Table II, application no. 3, preferably a homolog or functional equivalent as depicted in column 8 of Table II B, application no. 3, and being depicted in the same respective line as said B2066, and preferably the activity is increased mitochondrial.

Accordingly, in one embodiment, the molecule which activity is to be increased in the process of the invention is the gene product with an activity as a “uridine/cytidine kinase”, preferably it is the molecule of section (a) or (b) of this paragraph.

In particular, it was observed that in plants, especially in Arabidopsis thaliana, increasing or generating the activity of a gene product mitochondrial with the activity of a “uridine/cytidine kinase”, preferably being encoded by a gene comprising the nucleic acid sequence SEQ ID NO. 8937, preferably the coding region thereof, conferred the production of or the increase in 5-oxoproline compared with the wild type control.

The nucleic acid sequence of B2107 from Escherichia coli, e.g. as shown in the respective line in column 5 of Table I, application no. 3, has been published in B. And the activity of the gene product thereof is the activity of b2107-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “b2107-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2107, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2107, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2107, or a functional equivalent or a homolog thereof as depicted in column 8 of Table II, application no. 3, preferably a homolog or functional equivalent as depicted in column 8 of Table II B, application no. 3, and being depicted in the same respective line as said B2107, and preferably the activity is increased non-targeted.

Accordingly, in one embodiment, the molecule which activity is to be increased in the process of the invention is the gene product with an activity as a “b2107-protein”, preferably it is the molecule of section (a) or (b) of this paragraph.

In particular, it was observed that in plants, especially in Arabidopsis thaliana, increasing or generating the activity of a gene product non-targeted with the activity of a “b2107-protein”, preferably being encoded by a gene comprising the nucleic acid sequence SEQ ID NO. 39002, preferably the coding region thereof, conferred the production of or the increase in aspartate compared with the wild type control.

The nucleic acid sequence of B2178 from Escherichia coli, e.g. as shown in the respective line in column 5 of Table I, application no. 3, has been published in B. And the activity of the gene product thereof is the activity of ABC transporter permease protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “ABC transporter permease protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2178, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2178, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2178, or a functional equivalent or a homolog thereof as depicted in column 8 of Table II, application no. 3, preferably a homolog or functional equivalent as depicted in column 8 of Table II B, application no. 3, and being depicted in the same respective line as said B2178, and preferably the activity is increased non-targeted.

Accordingly, in one embodiment, the molecule which activity is to be increased in the process of the invention is the gene product with an activity as a “ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “ABC transporter permease protein”, preferably being encoded by a gene comprising the nucleic acid sequence SEQ ID NO. 39040, preferably the coding region thereof, conferred the production of or the increase in aspartate compared with the wild type control.

The nucleic acid sequence of B2178 from Escherichia coli, e.g. as shown in the respective line in column 5 of Table I, application no. 3, has been published in B. And the activity of the gene product thereof is the activity of ABC transporter permease protein.

Accordingly, in one embodiment, the process of the present invention for producing citrulline in a non-human organism, like a microorganism or a plant or a part thereof, comprises increasing or generating the activity of a gene product with the activity of a gene product conferring the activity of “ABC transporter permease protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2178, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2178, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2178, or a functional equivalent or a homolog thereof as depicted in column 8 of Table II, application no. 3, preferably a homolog or functional equivalent as depicted in column 8 of Table II B, application no. 3, and being depicted in the same respective line as said B2178, and preferably the activity is increased non-targeted.

Accordingly, in one embodiment, the molecule which activity is to be increased in the process of the invention is the gene product with an activity as a “ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “ABC transporter permease protein”, preferably being encoded by a gene comprising the nucleic acid sequence SEQ ID NO. 39040, preferably the coding region thereof, conferred the production of or the increase in citrulline compared with the wild type control.

The nucleic acid sequence of B2178 from Escherichia coli, e.g. as shown in the respective line in column 5 of Table I, application no. 3, has been published in B. And the activity of the gene product thereof is the activity of ABC transporter permease protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “ABC transporter permease protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2178, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2178, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2178, or a functional equivalent or a homolog thereof as depicted in column 8 of Table II, application no. 3, preferably a homolog or functional equivalent as depicted in column 8 of Table II B, application no. 3, and being depicted in the same respective line as said B2178, and preferably the activity is increased non-targeted.

Accordingly, in one embodiment, the molecule which activity is to be increased in the process of the invention is the gene product with an activity as a “ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “ABC transporter permease protein”, preferably being encoded by a gene comprising the nucleic acid sequence SEQ ID NO. 39040, preferably the coding region thereof, conferred the production of or the increase in phenylalanine compared with the wild type control.

The nucleic acid sequence of B2178 from Escherichia coli, e.g. as shown in the respective line in column 5 of Table I, application no. 3, has been published in B. And the activity of the gene product thereof is the activity of ABC transporter permease protein.

Accordingly, in one embodiment, the process of the present invention for producing serine in a non-human organism, like a microorganism or a plant or a part thereof, comprises increasing or generating the activity of a gene product with the activity of a gene product conferring the activity of “ABC transporter permease protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2178, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2178, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2178, or a functional equivalent or a homolog thereof as depicted in column 8 of Table II, application no. 3, preferably a homolog or functional equivalent as depicted in column 8 of Table II B, application no. 3, and being depicted in the same respective line as said B2178, and preferably the activity is increased non-targeted.

Accordingly, in one embodiment, the molecule which activity is to be increased in the process of the invention is the gene product with an activity as a “ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “ABC transporter permease protein”, preferably being encoded by a gene comprising the nucleic acid sequence SEQ ID NO. 39040, preferably the coding region thereof, conferred the production of or the increase in serine compared with the wild type control.

The nucleic acid sequence of B2178 from Escherichia coli, e.g. as shown in the respective line in column 5 of Table I, application no. 3, has been published in B. And the activity of the gene product thereof is the activity of ABC transporter permease protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “ABC transporter permease protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2178, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2178, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2178, or a functional equivalent or a homolog thereof as depicted in column 8 of Table II, application no. 3, preferably a homolog or functional equivalent as depicted in column 8 of Table II B, application no. 3, and being depicted in the same respective line as said B2178, and preferably the activity is increased non-targeted.

Accordingly, in one embodiment, the molecule which activity is to be increased in the process of the invention is the gene product with an activity as a “ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “ABC transporter permease protein”, preferably being encoded by a gene comprising the nucleic acid sequence SEQ ID NO. 39040, preferably the coding region thereof, conferred the production of or the increase in tyrosine compared with the wild type control.

The nucleic acid sequence of B2242 from Escherichia coli, e.g. as shown in the respective line in column 5 of Table I, application no. 3, has been published in B. And the activity of the gene product thereof is the activity of sn-glycerol-3-phosphate dehydrogenase.

Accordingly, in one embodiment, the process of the present invention for producing glycine in a non-human organism, like a microorganism or a plant or a part thereof, comprises increasing or generating the activity of a gene product with the activity of a gene product conferring the activity of “sn-glycerol-3-phosphate dehydrogenase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2242, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2242, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2242, or a functional equivalent or a homolog thereof as depicted in column 8 of Table II, application no. 3, preferably a homolog or functional equivalent as depicted in column 8 of Table II B, application no. 3, and being depicted in the same respective line as said B2242, and preferably the activity is increased plastidic.

Accordingly, in one embodiment, the molecule which activity is to be increased in the process of the invention is the gene product with an activity as a “sn-glycerol-3-phosphate dehydrogenase”, preferably it is the molecule of section (a) or (b) of this paragraph.

In particular, it was observed that in plants, especially in Arabidopsis thaliana, increasing or generating the activity of a gene product plastidic with the activity of a “sn-glycerol-3-phosphate dehydrogenase”, preferably being encoded by a gene comprising the nucleic acid sequence SEQ ID NO. 79279, preferably the coding region thereof, conferred the production of or the increase in glycine compared with the wild type control.

The nucleic acid sequence of B2281 from Escherichia coli, e.g. as shown in the respective line in column 5 of Table I, application no. 3, has been published in B. And the activity of the gene product thereof is the activity of NADH dehydrogenase I chain I.

Accordingly, in one embodiment, the process of the present invention for producing asparagine in a non-human organism, like a microorganism or a plant or a part thereof, comprises increasing or generating the activity of a gene product with the activity of a gene product conferring the activity of “NADH dehydrogenase I chain I”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2281, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2281, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2281, or a functional equivalent or a homolog thereof as depicted in column 8 of Table II, application no. 3, preferably a homolog or functional equivalent as depicted in column 8 of Table II B, application no. 3, and being depicted in the same respective line as said B2281, and preferably the activity is increased non-targeted.

Accordingly, in one embodiment, the molecule which activity is to be increased in the process of the invention is the gene product with an activity as a “NADH dehydrogenase I chain I”, preferably it is the molecule of section (a) or (b) of this paragraph.

In particular, it was observed that in plants, especially in Arabidopsis thaliana, increasing or generating the activity of a gene product non-targeted with the activity of a “NADH dehydrogenase I chain I”, preferably being encoded by a gene comprising the nucleic acid sequence SEQ ID NO. 39120, preferably the coding region thereof, conferred the production of or the increase in asparagine compared with the wild type control.

The nucleic acid sequence of B2360 from Escherichia coli, e.g. as shown in the respective line in column 5 of Table I, application no. 3, has been published in B. And the activity of the gene product thereof is the activity of b2360-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “b2360-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2360, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2360, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2360, or a functional equivalent or a homolog thereof as depicted in column 8 of Table II, application no. 3, preferably a homolog or functional equivalent as depicted in column 8 of Table II B, application no. 3, and being depicted in the same respective line as said B2360, and preferably the activity is increased non-targeted.

Accordingly, in one embodiment, the molecule which activity is to be increased in the process of the invention is the gene product with an activity as a “b2360-protein”, preferably it is the molecule of section (a) or (b) of this paragraph.

In particular, it was observed that in plants, especially in Arabidopsis thaliana, increasing or generating the activity of a gene product non-targeted with the activity of a “b2360-protein”, preferably being encoded by a gene comprising the nucleic acid sequence SEQ ID NO. 39219, preferably the coding region thereof, conferred the production of or the increase in aspartate compared with the wild type control.

The nucleic acid sequence of B2366 from Escherichia coli, e.g. as shown in the respective line in column 5 of Table I, application no. 3, has been published in B. And the activity of the gene product thereof is the activity of D-serine dehydratase.

Accordingly, in one embodiment, the process of the present invention for producing serine in a non-human organism, like a microorganism or a plant or a part thereof, comprises increasing or generating the activity of a gene product with the activity of a gene product conferring the activity of “D-serine dehydratase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2366, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2366, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2366, or a functional equivalent or a homolog thereof as depicted in column 8 of Table II, application no. 3, preferably a homolog or functional equivalent as depicted in column 8 of Table II B, application no. 3, and being depicted in the same respective line as said B2366, and preferably the activity is increased plastidic.

Accordingly, in one embodiment, the molecule which activity is to be increased in the process of the invention is the gene product with an activity as a “D-serine dehydratase”, preferably it is the molecule of section (a) or (b) of this paragraph.

In particular, it was observed that in plants, especially in Arabidopsis thaliana, increasing or generating the activity of a gene product plastidic with the activity of a “D-serine dehydratase”, preferably being encoded by a gene comprising the nucleic acid sequence SEQ ID NO. 79342, preferably the coding region thereof, conferred the production of or the increase in serine compared with the wild type control.

The nucleic acid sequence of B2399 from Escherichia coli, e.g. as shown in the respective line in column 5 of Table I, application no. 3, has been published in B. And the activity of the gene product thereof is the activity of b2399-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “b2399-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2399, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2399, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2399, or a functional equivalent or a homolog thereof as depicted in column 8 of Table II, application no. 3, preferably a homolog or functional equivalent as depicted in column 8 of Table II B, application no. 3, and being depicted in the same respective line as said B2399, and preferably the activity is increased non-targeted.

Accordingly, in one embodiment, the molecule which activity is to be increased in the process of the invention is the gene product with an activity as a “b2399-protein”, preferably it is the molecule of section (a) or (b) of this paragraph.

In particular, it was observed that in plants, especially in Arabidopsis thaliana, increasing or generating the activity of a gene product non-targeted with the activity of a “b2399-protein”, preferably being encoded by a gene comprising the nucleic acid sequence SEQ ID NO. 39237, preferably the coding region thereof, conferred the production of or the increase in tyrosine compared with the wild type control.

The nucleic acid sequence of B2399 from Escherichia coli, e.g. as shown in the respective line in column 5 of Table I, application no. 3, has been published in B. And the activity of the gene product thereof is the activity of b2399-protein.

Accordingly, in one embodiment, the process of the present invention for producing 3,4-dihydroxyphenylalanine (dopa) in a non-human organism, like a microorganism or a plant or a part thereof, comprises increasing or generating the activity of a gene product with the activity of a gene product conferring the activity of “b2399-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2399, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2399, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2399, or a functional equivalent or a homolog thereof as depicted in column 8 of Table II, application no. 3, preferably a homolog or functional equivalent as depicted in column 8 of Table II B, application no. 3, and being depicted in the same respective line as said B2399, and preferably the activity is increased non-targeted.

Accordingly, in one embodiment, the molecule which activity is to be increased in the process of the invention is the gene product with an activity as a “b2399-protein”, preferably it is the molecule of section (a) or (b) of this paragraph.

In particular, it was observed that in plants, especially in Arabidopsis thaliana, increasing or generating the activity of a gene product non-targeted with the activity of a “b2399-protein”, preferably being encoded by a gene comprising the nucleic acid sequence SEQ ID NO. 39237, preferably the coding region thereof, conferred the production of or the increase in 3,4-dihydroxyphenylalanine (dopa) compared with the wild type control.

The nucleic acid sequence of B2399 from Escherichia coli, e.g. as shown in the respective line in column 5 of Table I, application no. 3, has been published in B. And the activity of the gene product thereof is the activity of b2399-protein.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “b2399-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2399, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2399, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2399, or a functional equivalent or a homolog thereof as depicted in column 8 of Table II, application no. 3, preferably a homolog or functional equivalent as depicted in column 8 of Table II B, application no. 3, and being depicted in the same respective line as said B2399, and preferably the activity is increased non-targeted.

Accordingly, in one embodiment, the molecule which activity is to be increased in the process of the invention is the gene product with an activity as a “b2399-protein”, preferably it is the molecule of section (a) or (b) of this paragraph.

In particular, it was observed that in plants, especially in Arabidopsis thaliana, increasing or generating the activity of a gene product non-targeted with the activity of a “b2399-protein”, preferably being encoded by a gene comprising the nucleic acid sequence SEQ ID NO. 39237, preferably the coding region thereof, conferred the production of or the increase in 5-oxoproline compared with the wild type control.

The nucleic acid sequence of B2405 from Escherichia coli, e.g. as shown in the respective line in column 5 of Table I, application no. 3, has been published in B. And the activity of the gene product thereof is the activity of transcriptional regulator.

Accordingly, in one embodiment, the process of the present invention for producing alanine in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2405, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2405, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2405, or a functional equivalent or a homolog thereof as depicted in column 8 of Table II, application no. 3, preferably a homolog or functional equivalent as depicted in column 8 of Table II B, application no. 3, and being depicted in the same respective line as said B2405, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 39255, preferably the coding region thereof, conferred the production of or the increase in alanine compared with the wild type control.

The nucleic acid sequence of B2405 from Escherichia coli, e.g. as shown in the respective line in column 5 of Table I, application no. 3, has been published in B. And the activity of the gene product thereof is the activity of transcriptional regulator.

Accordingly, in one embodiment, the process of the present invention for producing aspartate in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2405, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2405, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2405, or a functional equivalent or a homolog thereof as depicted in column 8 of Table II, application no. 3, preferably a homolog or functional equivalent as depicted in column 8 of Table II B, application no. 3, and being depicted in the same respective line as said B2405, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 39255, preferably the coding region thereof, conferred the production of or the increase in aspartate compared with the wild type control.

The nucleic acid sequence of B2429 from Escherichia coli, e.g. as shown in the respective line in column 5 of Table I, application no. 3, has been published in B. And the activity of the gene product thereof is the activity of PTS system N-acetylmuramic acid-specific EIIBC component.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “PTS system N-acetylmuramic acid-specific EIIBC component”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2429, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2429, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2429, or a functional equivalent or a homolog thereof as depicted in column 8 of Table II, application no. 3, preferably a homolog or functional equivalent as depicted in column 8 of Table II B, application no. 3, and being depicted in the same respective line as said B2429, and preferably the activity is increased non-targeted.

Accordingly, in one embodiment, the molecule which activity is to be increased in the process of the invention is the gene product with an activity as a “PTS system N-acetylmuramic acidspecific EIIBC component”, preferably it is the molecule of section (a) or (b) of this paragraph.

In particular, it was observed that in plants, especially in Arabidopsis thaliana, increasing or generating the activity of a gene product non-targeted with the activity of a “PTS system Nacetylmuramic acid-specific EIIBC component”, preferably being encoded by a gene comprising the nucleic acid sequence SEQ ID NO. 79398, preferably the coding region thereof, conferred the production of or the increase in phenylalanine compared with the wild type control.

The nucleic acid sequence of B2474 from Escherichia coli, e.g. as shown in the respective line in column 5 of Table I, application no. 3, has been published in B. And the activity of the gene product thereof is the activity of b2474-protein.

Accordingly, in one embodiment, the process of the present invention for producing citrulline in a non-human organism, like a microorganism or a plant or a part thereof, comprises increasing or generating the activity of a gene product with the activity of a gene product conferring the activity of “b2474-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2474, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2474, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2474, or a functional equivalent or a homolog thereof as depicted in column 8 of Table II, application no. 3, preferably a homolog or functional equivalent as depicted in column 8 of Table II B, application no. 3, and being depicted in the same respective line as said B2474, and preferably the activity is increased non-targeted.

Accordingly, in one embodiment, the molecule which activity is to be increased in the process of the invention is the gene product with an activity as a “b2474-protein”, preferably it is the molecule of section (a) or (b) of this paragraph.

In particular, it was observed that in plants, especially in Arabidopsis thaliana, increasing or generating the activity of a gene product non-targeted with the activity of a “b2474-protein”, preferably being encoded by a gene comprising the nucleic acid sequence SEQ ID NO. 40329, preferably the coding region thereof, conferred the production of or the increase in citrulline compared with the wild type control.

The nucleic acid sequence of B2474 from Escherichia coli, e.g. as shown in the respective line in column 5 of Table I, application no. 3, has been published in B. And the activity of the gene product thereof is the activity of b2474-protein.

Accordingly, in one embodiment, the process of the present invention for producing glycine in a non-human organism, like a microorganism or a plant or a part thereof, comprises increasing or generating the activity of a gene product with the activity of a gene product conferring the activity of “b2474-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2474, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2474, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2474, or a functional equivalent or a homolog thereof as depicted in column 8 of Table II, application no. 3, preferably a homolog or functional equivalent as depicted in column 8 of Table II B, application no. 3, and being depicted in the same respective line as said B2474, and preferably the activity is increased non-targeted.

Accordingly, in one embodiment, the molecule which activity is to be increased in the process of the invention is the gene product with an activity as a “b2474-protein”, preferably it is the molecule of section (a) or (b) of this paragraph.

In particular, it was observed that in plants, especially in Arabidopsis thaliana, increasing or generating the activity of a gene product non-targeted with the activity of a “b2474-protein”, preferably being encoded by a gene comprising the nucleic acid sequence SEQ ID NO. 40329, preferably the coding region thereof, conferred the production of or the increase in glycine compared with the wild type control.

The nucleic acid sequence of B2482 from Escherichia coli, e.g. as shown in the respective line in column 5 of Table I, application no. 3, has been published in B. And the activity of the gene product thereof is the activity of NADH dehydrogenase subunit N.

Accordingly, in one embodiment, the process of the present invention for producing serine in a non-human organism, like a microorganism or a plant or a part thereof, comprises increasing or generating the activity of a gene product with the activity of a gene product conferring the activity of “NADH dehydrogenase subunit N”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2482, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2482, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2482, or a functional equivalent or a homolog thereof as depicted in column 8 of Table II, application no. 3, preferably a homolog or functional equivalent as depicted in column 8 of Table II B, application no. 3, and being depicted in the same respective line as said B2482, and preferably the activity is increased non-targeted.

Accordingly, in one embodiment, the molecule which activity is to be increased in the process of the invention is the gene product with an activity as a “NADH dehydrogenase subunit N”, preferably it is the molecule of section (a) or (b) of this paragraph.

In particular, it was observed that in plants, especially in Arabidopsis thaliana, increasing or generating the activity of a gene product non-targeted with the activity of a “NADH dehydrogenase subunit N”, preferably being encoded by a gene comprising the nucleic acid sequence SEQ ID NO. 79509, preferably the coding region thereof, conferred the production of or the increase in serine compared with the wild type control.

The nucleic acid sequence of B2482 from Escherichia coli, e.g. as shown in the respective line in column 5 of Table I, application no. 3, has been published in B. And the activity of the gene product thereof is the activity of NADH dehydrogenase subunit N.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “NADH dehydrogenase subunit N”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2482, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2482, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2482, or a functional equivalent or a homolog thereof as depicted in column 8 of Table II, application no. 3, preferably a homolog or functional equivalent as depicted in column 8 of Table II B, application no. 3, and being depicted in the same respective line as said B2482, and preferably the activity is increased non-targeted.

Accordingly, in one embodiment, the molecule which activity is to be increased in the process of the invention is the gene product with an activity as a “NADH dehydrogenase subunit N”, preferably it is the molecule of section (a) or (b) of this paragraph.

In particular, it was observed that in plants, especially in Arabidopsis thaliana, increasing or generating the activity of a gene product non-targeted with the activity of a “NADH dehydrogenase subunit N”, preferably being encoded by a gene comprising the nucleic acid sequence SEQ ID NO. 79509, preferably the coding region thereof, conferred the production of or the increase in 5-oxoproline compared with the wild type control.

The nucleic acid sequence of B2513 from Escherichia coli, e.g. as shown in the respective line in column 5 of Table I, application no. 3, has been published in B. And the activity of the gene product thereof is the activity of b2513-protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “b2513-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2513, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2513, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2513, or a functional equivalent or a homolog thereof as depicted in column 8 of Table II, application no. 3, preferably a homolog or functional equivalent as depicted in column 8 of Table II B, application no. 3, and being depicted in the same respective line as said B2513, and preferably the activity is increased non-targeted.

Accordingly, in one embodiment, the molecule which activity is to be increased in the process of the invention is the gene product with an activity as a “b2513-protein”, preferably it is the molecule of section (a) or (b) of this paragraph.

In particular, it was observed that in plants, especially in Arabidopsis thaliana, increasing or generating the activity of a gene product non-targeted with the activity of a “b2513-protein”, preferably being encoded by a gene comprising the nucleic acid sequence SEQ ID NO. 9167, preferably the coding region thereof, conferred the production of or the increase in phenylalanine compared with the wild type control.

The nucleic acid sequence of B2513 from Escherichia coli, e.g. as shown in the respective line in column 5 of Table I, application no. 3, has been published in B. And the activity of the gene product thereof is the activity of b2513-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “b2513-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2513, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2513, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2513, or a functional equivalent or a homolog thereof as depicted in column 8 of Table II, application no. 3, preferably a homolog or functional equivalent as depicted in column 8 of Table II B, application no. 3, and being depicted in the same respective line as said B2513, and preferably the activity is increased non-targeted.

Accordingly, in one embodiment, the molecule which activity is to be increased in the process of the invention is the gene product with an activity as a “b2513-protein”, preferably it is the molecule of section (a) or (b) of this paragraph.

In particular, it was observed that in plants, especially in Arabidopsis thaliana, increasing or generating the activity of a gene product non-targeted with the activity of a “b2513-protein”, preferably being encoded by a gene comprising the nucleic acid sequence SEQ ID NO. 9167, preferably the coding region thereof, conferred the production of or the increase in tyrosine compared with the wild type control.

The nucleic acid sequence of B2551 from Escherichia coli, e.g. as shown in the respective line in column 5 of Table I, application no. 3, has been published in B. And the activity of the gene product thereof is the activity of serine hydroxymethyltransferase.

Accordingly, in one embodiment, the process of the present invention for producing serine in a non-human organism, like a microorganism or a plant or a part thereof, comprises increasing or generating the activity of a gene product with the activity of a gene product conferring the activity of “serine hydroxymethyltransferase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2551, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2551, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2551, or a functional equivalent or a homolog thereof as depicted in column 8 of Table II, application no. 3, preferably a homolog or functional equivalent as depicted in column 8 of Table II B, application no. 3, and being depicted in the same respective line as said B2551, and preferably the activity is increased non-targeted.

Accordingly, in one embodiment, the molecule which activity is to be increased in the process of the invention is the gene product with an activity as a “serine hydroxymethyltransferase”, preferably it is the molecule of section (a) or (b) of this paragraph.

In particular, it was observed that in plants, especially in Arabidopsis thaliana, increasing or generating the activity of a gene product non-targeted with the activity of a “serine hydroxymethyltransferase”, preferably being encoded by a gene comprising the nucleic acid sequence SEQ ID NO. 79627, preferably the coding region thereof, conferred the production of or the increase in serine compared with the wild type control.

The nucleic acid sequence of B2600 from Escherichia coli, e.g. as shown in the respective line in column 5 of Table I, application no. 3, has been published in B. And the activity of the gene product thereof is the activity of chorismate mutase-T and prephenate dehydrogenase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “chorismate mutase-T and prephenate dehydrogenase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2600, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2600, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2600, or a functional equivalent or a homolog thereof as depicted in column 8 of Table II, application no. 3, preferably a homolog or functional equivalent as depicted in column 8 of Table II B, application no. 3, and being depicted in the same respective line as said B2600, and preferably the activity is increased plastidic.

Accordingly, in one embodiment, the molecule which activity is to be increased in the process of the invention is the gene product with an activity as a “chorismate mutase-T and prephenate dehydrogenase”, preferably it is the molecule of section (a) or (b) of this paragraph.

In particular, it was observed that in plants, especially in Arabidopsis thaliana, increasing or generating the activity of a gene product plastidic with the activity of a “chorismate mutase-T and prephenate dehydrogenase”, preferably being encoded by a gene comprising the nucleic acid sequence SEQ ID NO. 80521, preferably the coding region thereof, conferred the production of or the increase in phenylalanine compared with the wild type control.

The nucleic acid sequence of B2613 from Escherichia coli, e.g. as shown in the respective line in column 5 of Table I, application no. 3, has been published in B. And the activity of the gene product thereof is the activity of b2613-protein.

Accordingly, in one embodiment, the process of the present invention for producing alanine in a non-human organism, like a microorganism or a plant or a part thereof, comprises increasing or generating the activity of a gene product with the activity of a gene product conferring the activity of “b2613-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2613, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2613, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2613, or a functional equivalent or a homolog thereof as depicted in column 8 of Table II, application no. 3, preferably a homolog or functional equivalent as depicted in column 8 of Table II B, application no. 3, and being depicted in the same respective line as said B2613, and preferably the activity is increased non-targeted.

Accordingly, in one embodiment, the molecule which activity is to be increased in the process of the invention is the gene product with an activity as a “b2613-protein”, preferably it is the molecule of section (a) or (b) of this paragraph.

In particular, it was observed that in plants, especially in Arabidopsis thaliana, increasing or generating the activity of a gene product non-targeted with the activity of a “b2613-protein”, preferably being encoded by a gene comprising the nucleic acid sequence SEQ ID NO. 40665, preferably the coding region thereof, conferred the production of or the increase in alanine compared with the wild type control.

The nucleic acid sequence of B2634 from Escherichia coli, e.g. as shown in the respective line in column 5 of Table I, application no. 3, has been published in B. And the activity of the gene product thereof is the activity of transcriptional regulator.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2634, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2634, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2634, or a functional equivalent or a homolog thereof as depicted in column 8 of Table II, application no. 3, preferably a homolog or functional equivalent as depicted in column 8 of Table II B, application no. 3, and being depicted in the same respective line as said B2634, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 40726, preferably the coding region thereof, conferred the production of or the increase in phenylalanine compared with the wild type control.

The nucleic acid sequence of B2634 from Escherichia coli, e.g. as shown in the respective line in column 5 of Table I, application no. 3, has been published in B. And the activity of the gene product thereof is the activity of transcriptional regulator.

Accordingly, in one embodiment, the process of the present invention for producing tyrosine in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2634, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2634, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2634, or a functional equivalent or a homolog thereof as depicted in column 8 of Table II, application no. 3, preferably a homolog or functional equivalent as depicted in column 8 of Table II B, application no. 3, and being depicted in the same respective line as said B2634, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 40726, preferably the coding region thereof, conferred the production of or the increase in tyrosine compared with the wild type control.

The nucleic acid sequence of B2673 from Escherichia coli, e.g. as shown in the respective line in column 5 of Table I, application no. 3, has been published in B. And the activity of the gene product thereof is the activity of b2673-protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “b2673-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2673, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2673, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2673, or a functional equivalent or a homolog thereof as depicted in column 8 of Table II, application no. 3, preferably a homolog or functional equivalent as depicted in column 8 of Table II B, application no. 3, and being depicted in the same respective line as said B2673, and preferably the activity is increased non-targeted.

Accordingly, in one embodiment, the molecule which activity is to be increased in the process of the invention is the gene product with an activity as a “b2673-protein”, preferably it is the molecule of section (a) or (b) of this paragraph.

In particular, it was observed that in plants, especially in Arabidopsis thaliana, increasing or generating the activity of a gene product non-targeted with the activity of a “b2673-protein”, preferably being encoded by a gene comprising the nucleic acid sequence SEQ ID NO. 9244, preferably the coding region thereof, conferred the production of or the increase in phenylalanine compared with the wild type control.

The nucleic acid sequence of B2673 from Escherichia coli, e.g. as shown in the respective line in column 5 of Table I, application no. 3, has been published in B. And the activity of the gene product thereof is the activity of b2673-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “b2673-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2673, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2673, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2673, or a functional equivalent or a homolog thereof as depicted in column 8 of Table II, application no. 3, preferably a homolog or functional equivalent as depicted in column 8 of Table II B, application no. 3, and being depicted in the same respective line as said B2673, and preferably the activity is increased non-targeted.

Accordingly, in one embodiment, the molecule which activity is to be increased in the process of the invention is the gene product with an activity as a “b2673-protein”, preferably it is the molecule of section (a) or (b) of this paragraph.

In particular, it was observed that in plants, especially in Arabidopsis thaliana, increasing or generating the activity of a gene product non-targeted with the activity of a “b2673-protein”, preferably being encoded by a gene comprising the nucleic acid sequence SEQ ID NO. 9244, preferably the coding region thereof, conferred the production of or the increase in tyrosine compared with the wild type control.

The nucleic acid sequence of B2680 from Escherichia coli, e.g. as shown in the respective line in column 5 of Table I, application no. 3, has been published in B. And the activity of the gene product thereof is the activity of transport protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “transport protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2680, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2680, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2680, or a functional equivalent or a homolog thereof as depicted in column 8 of Table II, application no. 3, preferably a homolog or functional equivalent as depicted in column 8 of Table II B, application no. 3, and being depicted in the same respective line as said B2680, and preferably the activity is increased non-targeted.

Accordingly, in one embodiment, the molecule which activity is to be increased in the process of the invention is the gene product with an activity as a “transport protein”, preferably it is the molecule of section (a) or (b) of this paragraph.

In particular, it was observed that in plants, especially in Arabidopsis thaliana, increasing or generating the activity of a gene product non-targeted with the activity of a “transport protein”, preferably being encoded by a gene comprising the nucleic acid sequence SEQ ID NO. 80589, preferably the coding region thereof, conferred the production of or the increase in aspartate compared with the wild type control.

The nucleic acid sequence of B2701 from Escherichia coli, e.g. as shown in the respective line in column 5 of Table I, application no. 3, has been published in B. And the activity of the gene product thereof is the activity of murein transglycosylase.

Accordingly, in one embodiment, the process of the present invention for producing alanine in a non-human organism, like a microorganism or a plant or a part thereof, comprises increasing or generating the activity of a gene product with the activity of a gene product conferring the activity of “murein transglycosylase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2701, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2701, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2701, or a functional equivalent or a homolog thereof as depicted in column 8 of Table II, application no. 3, preferably a homolog or functional equivalent as depicted in column 8 of Table II B, application no. 3, and being depicted in the same respective line as said B2701, and preferably the activity is increased non-targeted.

Accordingly, in one embodiment, the molecule which activity is to be increased in the process of the invention is the gene product with an activity as a “murein transglycosylase”, preferably it is the molecule of section (a) or (b) of this paragraph.

In particular, it was observed that in plants, especially in Arabidopsis thaliana, increasing or generating the activity of a gene product non-targeted with the activity of a “murein transglycosylase”, preferably being encoded by a gene comprising the nucleic acid sequence SEQ ID NO. 80756, preferably the coding region thereof, conferred the production of or the increase in alanine compared with the wild type control.

The nucleic acid sequence of B2701 from Escherichia coli, e.g. as shown in the respective line in column 5 of Table I, application no. 3, has been published in B. And the activity of the gene product thereof is the activity of murein transglycosylase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “murein transglycosylase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2701, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2701, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2701, or a functional equivalent or a homolog thereof as depicted in column 8 of Table II, application no. 3, preferably a homolog or functional equivalent as depicted in column 8 of Table II B, application no. 3, and being depicted in the same respective line as said B2701, and preferably the activity is increased non-targeted.

Accordingly, in one embodiment, the molecule which activity is to be increased in the process of the invention is the gene product with an activity as a “murein transglycosylase”, preferably it is the molecule of section (a) or (b) of this paragraph.

In particular, it was observed that in plants, especially in Arabidopsis thaliana, increasing or generating the activity of a gene product non-targeted with the activity of a “murein transglycosylase”, preferably being encoded by a gene comprising the nucleic acid sequence SEQ ID NO. 80756, preferably the coding region thereof, conferred the production of or the increase in phenylalanine compared with the wild type control.

The nucleic acid sequence of B2701 from Escherichia coli, e.g. as shown in the respective line in column 5 of Table I, application no. 3, has been published in B. And the activity of the gene product thereof is the activity of murein transglycosylase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “murein transglycosylase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2701, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2701, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2701, or a functional equivalent or a homolog thereof as depicted in column 8 of Table II, application no. 3, preferably a homolog or functional equivalent as depicted in column 8 of Table II B, application no. 3, and being depicted in the same respective line as said B2701, and preferably the activity is increased non-targeted.

Accordingly, in one embodiment, the molecule which activity is to be increased in the process of the invention is the gene product with an activity as a “murein transglycosylase”, preferably it is the molecule of section (a) or (b) of this paragraph.

In particular, it was observed that in plants, especially in Arabidopsis thaliana, increasing or generating the activity of a gene product non-targeted with the activity of a “murein transglycosylase”, preferably being encoded by a gene comprising the nucleic acid sequence SEQ ID NO. 80756, preferably the coding region thereof, conferred the production of or the increase in tyrosine compared with the wild type control.

The nucleic acid sequence of B2714 from Escherichia coli, e.g. as shown in the respective line in column 5 of Table I, application no. 3, has been published in B. And the activity of the gene product thereof is the activity of transcriptional regulator.

Accordingly, in one embodiment, the process of the present invention for producing alanine in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2714, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2714, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2714, or a functional equivalent or a homolog thereof as depicted in column 8 of Table II, application no. 3, preferably a homolog or functional equivalent as depicted in column 8 of Table II B, application no. 3, and being depicted in the same respective line as said B2714, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 40741, preferably the coding region thereof, conferred the production of or the increase in alanine compared with the wild type control.

The nucleic acid sequence of B2739 from Escherichia coli, e.g. as shown in the respective line in column 5 of Table I, application no. 3, has been published in B. And the activity of the gene product thereof is the activity of b2739-protein.

Accordingly, in one embodiment, the process of the present invention for producing alanine in a non-human organism, like a microorganism or a plant or a part thereof, comprises increasing or generating the activity of a gene product with the activity of a gene product conferring the activity of “b2739-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2739, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2739, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2739, or a functional equivalent or a homolog thereof as depicted in column 8 of Table II, application no. 3, preferably a homolog or functional equivalent as depicted in column 8 of Table II B, application no. 3, and being depicted in the same respective line as said B2739, and preferably the activity is increased non-targeted.

Accordingly, in one embodiment, the molecule which activity is to be increased in the process of the invention is the gene product with an activity as a “b2739-protein”, preferably it is the molecule of section (a) or (b) of this paragraph.

In particular, it was observed that in plants, especially in Arabidopsis thaliana, increasing or generating the activity of a gene product non-targeted with the activity of a “b2739-protein”, preferably being encoded by a gene comprising the nucleic acid sequence SEQ ID NO. 80906, preferably the coding region thereof, conferred the production of or the increase in alanine compared with the wild type control.

The nucleic acid sequence of B2739 from Escherichia coli, e.g. as shown in the respective line in column 5 of Table I, application no. 3, has been published in B. And the activity of the gene product thereof is the activity of b2739-protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “b2739-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2739, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2739, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2739, or a functional equivalent or a homolog thereof as depicted in column 8 of Table II, application no. 3, preferably a homolog or functional equivalent as depicted in column 8 of Table II B, application no. 3, and being depicted in the same respective line as said B2739, and preferably the activity is increased non-targeted.

Accordingly, in one embodiment, the molecule which activity is to be increased in the process of the invention is the gene product with an activity as a “b2739-protein”, preferably it is the molecule of section (a) or (b) of this paragraph.

In particular, it was observed that in plants, especially in Arabidopsis thaliana, increasing or generating the activity of a gene product non-targeted with the activity of a “b2739-protein”, preferably being encoded by a gene comprising the nucleic acid sequence SEQ ID NO. 80906, preferably the coding region thereof, conferred the production of or the increase in phenylalanine compared with the wild type control.

The nucleic acid sequence of B2739 from Escherichia coli, e.g. as shown in the respective line in column 5 of Table I, application no. 3, has been published in B. And the activity of the gene product thereof is the activity of b2739-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “b2739-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2739, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2739, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2739, or a functional equivalent or a homolog thereof as depicted in column 8 of Table II, application no. 3, preferably a homolog or functional equivalent as depicted in column 8 of Table II B, application no. 3, and being depicted in the same respective line as said B2739, and preferably the activity is increased non-targeted.

Accordingly, in one embodiment, the molecule which activity is to be increased in the process of the invention is the gene product with an activity as a “b2739-protein”, preferably it is the molecule of section (a) or (b) of this paragraph.

In particular, it was observed that in plants, especially in Arabidopsis thaliana, increasing or generating the activity of a gene product non-targeted with the activity of a “b2739-protein”, preferably being encoded by a gene comprising the nucleic acid sequence SEQ ID NO. 80906, preferably the coding region thereof, conferred the production of or the increase in tyrosine compared with the wild type control.

The nucleic acid sequence of B2747 from Escherichia coli, e.g. as shown in the respective line in column 5 of Table I, application no. 3, has been published in B. And the activity of the gene product thereof is the activity of 2-C-methyl-D-erythritol 4-phosphate cytidylyltransferase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “2-C-methyl-D-erythritol 4-phosphate cytidylyltransferase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2747, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2747, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2747, or a functional equivalent or a homolog thereof as depicted in column 8 of Table II, application no. 3, preferably a homolog or functional equivalent as depicted in column 8 of Table II B, application no. 3, and being depicted in the same respective line as said B2747, and preferably the activity is increased non-targeted.

Accordingly, in one embodiment, the molecule which activity is to be increased in the process of the invention is the gene product with an activity as a “2-C-methyl-D-erythritol 4-phosphate cytidylyltransferase”, preferably it is the molecule of section (a) or (b) of this paragraph.

In particular, it was observed that in plants, especially in Arabidopsis thaliana, increasing or generating the activity of a gene product non-targeted with the activity of a “2-C-methyl-D-erythritol 4-phosphate cytidylyltransferase”, preferably being encoded by a gene comprising the nucleic acid sequence SEQ ID NO. 81075, preferably the coding region thereof, conferred the production of or the increase in tyrosine compared with the wild type control.

The nucleic acid sequence of B2747 from Escherichia coli, e.g. as shown in the respective line in column 5 of Table I, application no. 3, has been published in B. And the activity of the gene product thereof is the activity of 2-C-methyl-D-erythritol 4-phosphate cytidylyltransferase.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “2-C-methyl-D-erythritol 4-phosphate cytidylyltransferase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2747, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2747, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2747, or a functional equivalent or a homolog thereof as depicted in column 8 of Table II, application no. 3, preferably a homolog or functional equivalent as depicted in column 8 of Table II B, application no. 3, and being depicted in the same respective line as said B2747, and preferably the activity is increased non-targeted.

Accordingly, in one embodiment, the molecule which activity is to be increased in the process of the invention is the gene product with an activity as a “2-C-methyl-D-erythritol 4-phosphate cytidylyltransferase”, preferably it is the molecule of section (a) or (b) of this paragraph.

In particular, it was observed that in plants, especially in Arabidopsis thaliana, increasing or generating the activity of a gene product non-targeted with the activity of a “2-C-methyl-Derythritol 4-phosphate cytidylyltransferase”, preferably being encoded by a gene comprising the nucleic acid sequence SEQ ID NO. 81075, preferably the coding region thereof, conferred the production of or the increase in 5-oxoproline compared with the wild type control.

The nucleic acid sequence of B2762 from Escherichia coli, e.g. as shown in the respective line in column 5 of Table I, application no. 3, has been published in B. And the activity of the gene product thereof is the activity of phosphoadenosine phosphosulfate reductase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “phosphoadenosine phosphosulfate reductase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2762, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2762, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2762, or a functional equivalent or a homolog thereof as depicted in column 8 of Table II, application no. 3, preferably a homolog or functional equivalent as depicted in column 8 of Table II B, application no. 3, and being depicted in the same respective line as said B2762, and preferably the activity is increased non-targeted.

Accordingly, in one embodiment, the molecule which activity is to be increased in the process of the invention is the gene product with an activity as a “phosphoadenosine phosphosulfate reductase”, preferably it is the molecule of section (a) or (b) of this paragraph.

In particular, it was observed that in plants, especially in Arabidopsis thaliana, increasing or generating the activity of a gene product non-targeted with the activity of a “phosphoadenosine phosphosulfate reductase”, preferably being encoded by a gene comprising the nucleic acid sequence SEQ ID NO. 81451, preferably the coding region thereof, conferred the production of or the increase in phenylalanine compared with the wild type control.

The nucleic acid sequence of B2812 from Escherichia coli, e.g. as shown in the respective line in column 5 of Table I, application no. 3, has been published in B. And the activity of the gene product thereof is the activity of b2812-protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “b2812-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2812, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2812, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2812, or a functional equivalent or a homolog thereof as depicted in column 8 of Table II, application no. 3, preferably a homolog or functional equivalent as depicted in column 8 of Table II B, application no. 3, and being depicted in the same respective line as said B2812, and preferably the activity is increased non-targeted.

Accordingly, in one embodiment, the molecule which activity is to be increased in the process of the invention is the gene product with an activity as a “b2812-protein”, preferably it is the molecule of section (a) or (b) of this paragraph.

In particular, it was observed that in plants, especially in Arabidopsis thaliana, increasing or generating the activity of a gene product non-targeted with the activity of a “b2812-protein”, preferably being encoded by a gene comprising the nucleic acid sequence SEQ ID NO. 40795, preferably the coding region thereof, conferred the production of or the increase in phenylalanine compared with the wild type control.

The nucleic acid sequence of B2812 from Escherichia coli, e.g. as shown in the respective line in column 5 of Table I, application no. 3, has been published in B. And the activity of the gene product thereof is the activity of b2812-protein.

Accordingly, in one embodiment, the process of the present invention for producing serine in a non-human organism, like a microorganism or a plant or a part thereof, comprises increasing or generating the activity of a gene product with the activity of a gene product conferring the activity of “b2812-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2812, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2812, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2812, or a functional equivalent or a homolog thereof as depicted in column 8 of Table II, application no. 3, preferably a homolog or functional equivalent as depicted in column 8 of Table II B, application no. 3, and being depicted in the same respective line as said B2812, and preferably the activity is increased non-targeted.

Accordingly, in one embodiment, the molecule which activity is to be increased in the process of the invention is the gene product with an activity as a “b2812-protein”, preferably it is the molecule of section (a) or (b) of this paragraph.

In particular, it was observed that in plants, especially in Arabidopsis thaliana, increasing or generating the activity of a gene product non-targeted with the activity of a “b2812-protein”, preferably being encoded by a gene comprising the nucleic acid sequence SEQ ID NO. 40795, preferably the coding region thereof, conferred the production of or the increase in serine compared with the wild type control.

The nucleic acid sequence of B2812 from Escherichia coli, e.g. as shown in the respective line in column 5 of Table I, application no. 3, has been published in B. And the activity of the gene product thereof is the activity of b2812-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “b2812-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2812, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2812, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2812, or a functional equivalent or a homolog thereof as depicted in column 8 of Table II, application no. 3, preferably a homolog or functional equivalent as depicted in column 8 of Table II B, application no. 3, and being depicted in the same respective line as said B2812, and preferably the activity is increased non-targeted.

Accordingly, in one embodiment, the molecule which activity is to be increased in the process of the invention is the gene product with an activity as a “b2812-protein”, preferably it is the molecule of section (a) or (b) of this paragraph.

In particular, it was observed that in plants, especially in Arabidopsis thaliana, increasing or generating the activity of a gene product non-targeted with the activity of a “b2812-protein”, preferably being encoded by a gene comprising the nucleic acid sequence SEQ ID NO. 40795, preferably the coding region thereof, conferred the production of or the increase in tyrosine compared with the wild type control.

The nucleic acid sequence of B2818 from Escherichia coli, e.g. as shown in the respective line in column 5 of Table I, application no. 3, has been published in B. And the activity of the gene product thereof is the activity of amino-acid acetyltransferase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “amino-acid acetyltransferase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2818, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2818, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2818, or a functional equivalent or a homolog thereof as depicted in column 8 of Table II, application no. 3, preferably a homolog or functional equivalent as depicted in column 8 of Table II B, application no. 3, and being depicted in the same respective line as said B2818, and preferably the activity is increased non-targeted.

Accordingly, in one embodiment, the molecule which activity is to be increased in the process of the invention is the gene product with an activity as a “amino-acid acetyltransferase”, preferably it is the molecule of section (a) or (b) of this paragraph.

In particular, it was observed that in plants, especially in Arabidopsis thaliana, increasing or generating the activity of a gene product non-targeted with the activity of a “amino-acid acetyltransferase”, preferably being encoded by a gene comprising the nucleic acid sequence SEQ ID NO. 81576, preferably the coding region thereof, conferred the production of or the increase in phenylalanine compared with the wild type control.

The nucleic acid sequence of B2818 from Escherichia coli, e.g. as shown in the respective line in column 5 of Table I, application no. 3, has been published in B. And the activity of the gene product thereof is the activity of amino-acid acetyltransferase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “amino-acid acetyltransferase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2818, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2818, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2818, or a functional equivalent or a homolog thereof as depicted in column 8 of Table II, application no. 3, preferably a homolog or functional equivalent as depicted in column 8 of Table II B, application no. 3, and being depicted in the same respective line as said B2818, and preferably the activity is increased non-targeted.

Accordingly, in one embodiment, the molecule which activity is to be increased in the process of the invention is the gene product with an activity as a “amino-acid acetyltransferase”, preferably it is the molecule of section (a) or (b) of this paragraph.

In particular, it was observed that in plants, especially in Arabidopsis thaliana, increasing or generating the activity of a gene product non-targeted with the activity of a “amino-acid acetyltransferase”, preferably being encoded by a gene comprising the nucleic acid sequence SEQ ID NO. 81576, preferably the coding region thereof, conferred the production of or the increase in tyrosine compared with the wild type control.

The nucleic acid sequence of B2818 from Escherichia coli, e.g. as shown in the respective line in column 5 of Table I, application no. 3, has been published in B. And the activity of the gene product thereof is the activity of amino-acid acetyltransferase.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “amino-acid acetyltransferase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2818, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2818, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2818, or a functional equivalent or a homolog thereof as depicted in column 8 of Table II, application no. 3, preferably a homolog or functional equivalent as depicted in column 8 of Table II B, application no. 3, and being depicted in the same respective line as said B2818, and preferably the activity is increased non-targeted.

Accordingly, in one embodiment, the molecule which activity is to be increased in the process of the invention is the gene product with an activity as a “amino-acid acetyltransferase”, preferably it is the molecule of section (a) or (b) of this paragraph.

In particular, it was observed that in plants, especially in Arabidopsis thaliana, increasing or generating the activity of a gene product non-targeted with the activity of a “amino-acid acetyltransferase”, preferably being encoded by a gene comprising the nucleic acid sequence SEQ ID NO. 81576, preferably the coding region thereof, conferred the production of or the increase in 5-oxoproline compared with the wild type control.

The nucleic acid sequence of B2828 from Escherichia coli, e.g. as shown in the respective line in column 5 of Table I, application no. 3, has been published in B. And the activity of the gene product thereof is the activity of prolipoprotein diacylglyceryl transferase.

Accordingly, in one embodiment, the process of the present invention for producing aspartate in a non-human organism, like a microorganism or a plant or a part thereof, comprises increasing or generating the activity of a gene product with the activity of a gene product conferring the activity of “prolipoprotein diacylglyceryl transferase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2828, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2828, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2828, or a functional equivalent or a homolog thereof as depicted in column 8 of Table II, application no. 3, preferably a homolog or functional equivalent as depicted in column 8 of Table II B, application no. 3, and being depicted in the same respective line as said B2828, and preferably the activity is increased non-targeted.

Accordingly, in one embodiment, the molecule which activity is to be increased in the process of the invention is the gene product with an activity as a “prolipoprotein diacylglyceryl transferase”, preferably it is the molecule of section (a) or (b) of this paragraph.

In particular, it was observed that in plants, especially in Arabidopsis thaliana, increasing or generating the activity of a gene product non-targeted with the activity of a “prolipoprotein diacylglyceryl transferase”, preferably being encoded by a gene comprising the nucleic acid sequence SEQ ID NO. 81692, preferably the coding region thereof, conferred the production of or the increase in aspartate compared with the wild type control.

The nucleic acid sequence of B2839 from Escherichia coli, e.g. as shown in the respective line in column 5 of Table I, application no. 3, has been published in B. And the activity of the gene product thereof is the activity of transcriptional regulator.

Accordingly, in one embodiment, the process of the present invention for producing tyrosine in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2839, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2839, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2839, or a functional equivalent or a homolog thereof as depicted in column 8 of Table II, application no. 3, preferably a homolog or functional equivalent as depicted in column 8 of Table II B, application no. 3, and being depicted in the same respective line as said B2839, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 81935, preferably the coding region thereof, conferred the production of or the increase in tyrosine compared with the wild type control.

The nucleic acid sequence of B2849 from Escherichia coli, e.g. as shown in the respective line in column 5 of Table I, application no. 3, has been published in B. And the activity of the gene product thereof is the activity of b2849-protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “b2849-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2849, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2849, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2849, or a functional equivalent or a homolog thereof as depicted in column 8 of Table II, application no. 3, preferably a homolog or functional equivalent as depicted in column 8 of Table II B, application no. 3, and being depicted in the same respective line as said B2849, and preferably the activity is increased non-targeted.

Accordingly, in one embodiment, the molecule which activity is to be increased in the process of the invention is the gene product with an activity as a “b2849-protein”, preferably it is the molecule of section (a) or (b) of this paragraph.

In particular, it was observed that in plants, especially in Arabidopsis thaliana, increasing or generating the activity of a gene product non-targeted with the activity of a “b2849-protein”, preferably being encoded by a gene comprising the nucleic acid sequence SEQ ID NO. 81980, preferably the coding region thereof, conferred the production of or the increase in phenylalanine compared with the wild type control.

The nucleic acid sequence of B2913 from Escherichia coli, e.g. as shown in the respective line in column 5 of Table I, application no. 3, has been published in B. And the activity of the gene product thereof is the activity of D-3-phosphoglycerate dehydrogenase.

Accordingly, in one embodiment, the process of the present invention for producing glycine in a non-human organism, like a microorganism or a plant or a part thereof, comprises increasing or generating the activity of a gene product with the activity of a gene product conferring the activity of “D-3-phosphoglycerate dehydrogenase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2913, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2913, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2913, or a functional equivalent or a homolog thereof as depicted in column 8 of Table II, application no. 3, preferably a homolog or functional equivalent as depicted in column 8 of Table II B, application no. 3, and being depicted in the same respective line as said B2913, and preferably the activity is increased plastidic.

Accordingly, in one embodiment, the molecule which activity is to be increased in the process of the invention is the gene product with an activity as a “D-3-phosphoglycerate dehydrogenase”, preferably it is the molecule of section (a) or (b) of this paragraph.

In particular, it was observed that in plants, especially in Arabidopsis thaliana, increasing or generating the activity of a gene product plastidic with the activity of a “D-3-phosphoglycerate dehydrogenase”, preferably being encoded by a gene comprising the nucleic acid sequence SEQ ID NO. 81988, preferably the coding region thereof, conferred the production of or the increase in glycine compared with the wild type control.

The nucleic acid sequence of B2913 from Escherichia coli, e.g. as shown in the respective line in column 5 of Table I, application no. 3, has been published in B. And the activity of the gene product thereof is the activity of D-3-phosphoglycerate dehydrogenase.

Accordingly, in one embodiment, the process of the present invention for producing serine in a non-human organism, like a microorganism or a plant or a part thereof, comprises increasing or generating the activity of a gene product with the activity of a gene product conferring the activity of “D-3-phosphoglycerate dehydrogenase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2913, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2913, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2913, or a functional equivalent or a homolog thereof as depicted in column 8 of Table II, application no. 3, preferably a homolog or functional equivalent as depicted in column 8 of Table II B, application no. 3, and being depicted in the same respective line as said B2913, and preferably the activity is increased plastidic.

Accordingly, in one embodiment, the molecule which activity is to be increased in the process of the invention is the gene product with an activity as a “D-3-phosphoglycerate dehydrogenase”, preferably it is the molecule of section (a) or (b) of this paragraph.

In particular, it was observed that in plants, especially in Arabidopsis thaliana, increasing or generating the activity of a gene product plastidic with the activity of a “D-3-phosphoglycerate dehydrogenase”, preferably being encoded by a gene comprising the nucleic acid sequence SEQ ID NO. 81988, preferably the coding region thereof, conferred the production of or the increase in serine compared with the wild type control.

The nucleic acid sequence of B2923 from Escherichia coli, e.g. as shown in the respective line in column 5 of Table I, application no. 3, has been published in B. And the activity of the gene product thereof is the activity of arginine exporter protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “arginine exporter protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B2923, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B2923, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B2923, or a functional equivalent or a homolog thereof as depicted in column 8 of Table II, application no. 3, preferably a homolog or functional equivalent as depicted in column 8 of Table II B, application no. 3, and being depicted in the same respective line as said B2923, and preferably the activity is increased non-targeted.

Accordingly, in one embodiment, the molecule which activity is to be increased in the process of the invention is the gene product with an activity as a “arginine exporter protein”, preferably it is the molecule of section (a) or (b) of this paragraph.

In particular, it was observed that in plants, especially in Arabidopsis thaliana, increasing or generating the activity of a gene product non-targeted with the activity of a “arginine exporter protein”, preferably being encoded by a gene comprising the nucleic acid sequence SEQ ID NO. 9333, preferably the coding region thereof, conferred the production of or the increase in aspartate compared with the wild type control.

The nucleic acid sequence of B3064 from Escherichia coli, e.g. as shown in the respective line in column 5 of Table I, application no. 3, has been published in B. And the activity of the gene product thereof is the activity of glycoprotease.

Accordingly, in one embodiment, the process of the present invention for producing asparagine in a non-human organism, like a microorganism or a plant or a part thereof, comprises increasing or generating the activity of a gene product with the activity of a gene product conferring the activity of “glycoprotease”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3064, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3064, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3064, or a functional equivalent or a homolog thereof as depicted in column 8 of Table II, application no. 3, preferably a homolog or functional equivalent as depicted in column 8 of Table II B, application no. 3, and being depicted in the same respective line as said B3064, and preferably the activity is increased non-targeted.

Accordingly, in one embodiment, the molecule which activity is to be increased in the process of the invention is the gene product with an activity as a “glycoprotease”, preferably it is the molecule of section (a) or (b) of this paragraph.

In particular, it was observed that in plants, especially in Arabidopsis thaliana, increasing or generating the activity of a gene product non-targeted with the activity of a “glycoprotease”, preferably being encoded by a gene comprising the nucleic acid sequence SEQ ID NO. 42046, preferably the coding region thereof, conferred the production of or the increase in asparagine compared with the wild type control.

The nucleic acid sequence of B3083 from Escherichia coli, e.g. as shown in the respective line in column 5 of Table I, application no. 3, has been published in B. And the activity of the gene product thereof is the activity of b3083-protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “b3083-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3083, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3083, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3083, or a functional equivalent or a homolog thereof as depicted in column 8 of Table II, application no. 3, preferably a homolog or functional equivalent as depicted in column 8 of Table II B, application no. 3, and being depicted in the same respective line as said B3083, and preferably the activity is increased non-targeted.

Accordingly, in one embodiment, the molecule which activity is to be increased in the process of the invention is the gene product with an activity as a “b3083-protein”, preferably it is the molecule of section (a) or (b) of this paragraph.

In particular, it was observed that in plants, especially in Arabidopsis thaliana, increasing or generating the activity of a gene product non-targeted with the activity of a “b3083-protein”, preferably being encoded by a gene comprising the nucleic acid sequence SEQ ID NO. 82424, preferably the coding region thereof, conferred the production of or the increase in phenylalanine compared with the wild type control.

The nucleic acid sequence of B3083 from Escherichia coli, e.g. as shown in the respective line in column 5 of Table I, application no. 3, has been published in B. And the activity of the gene product thereof is the activity of b3083-protein.

Accordingly, in one embodiment, the process of the present invention for producing serine in a non-human organism, like a microorganism or a plant or a part thereof, comprises increasing or generating the activity of a gene product with the activity of a gene product conferring the activity of “b3083-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3083, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3083, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3083, or a functional equivalent or a homolog thereof as depicted in column 8 of Table II, application no. 3, preferably a homolog or functional equivalent as depicted in column 8 of Table II B, application no. 3, and being depicted in the same respective line as said B3083, and preferably the activity is increased non-targeted.

Accordingly, in one embodiment, the molecule which activity is to be increased in the process of the invention is the gene product with an activity as a “b3083-protein”, preferably it is the molecule of section (a) or (b) of this paragraph.

In particular, it was observed that in plants, especially in Arabidopsis thaliana, increasing or generating the activity of a gene product non-targeted with the activity of a “b3083-protein”, preferably being encoded by a gene comprising the nucleic acid sequence SEQ ID NO. 82424, preferably the coding region thereof, conferred the production of or the increase in serine compared with the wild type control.

The nucleic acid sequence of B3083 from Escherichia coli, e.g. as shown in the respective line in column 5 of Table I, application no. 3, has been published in B. And the activity of the gene product thereof is the activity of b3083-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “b3083-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3083, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3083, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3083, or a functional equivalent or a homolog thereof as depicted in column 8 of Table II, application no. 3, preferably a homolog or functional equivalent as depicted in column 8 of Table II B, application no. 3, and being depicted in the same respective line as said B3083, and preferably the activity is increased non-targeted.

Accordingly, in one embodiment, the molecule which activity is to be increased in the process of the invention is the gene product with an activity as a “b3083-protein”, preferably it is the molecule of section (a) or (b) of this paragraph.

In particular, it was observed that in plants, especially in Arabidopsis thaliana, increasing or generating the activity of a gene product non-targeted with the activity of a “b3083-protein”, preferably being encoded by a gene comprising the nucleic acid sequence SEQ ID NO. 82424, preferably the coding region thereof, conferred the production of or the increase in tyrosine compared with the wild type control.

The nucleic acid sequence of B3098 from Escherichia coli, e.g. as shown in the respective line in column 5 of Table I, application no. 3, has been published in B. And the activity of the gene product thereof is the activity of b3098-protein.

Accordingly, in one embodiment, the process of the present invention for producing alanine in a non-human organism, like a microorganism or a plant or a part thereof, comprises increasing or generating the activity of a gene product with the activity of a gene product conferring the activity of “b3098-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3098, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3098, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3098, or a functional equivalent or a homolog thereof as depicted in column 8 of Table II, application no. 3, preferably a homolog or functional equivalent as depicted in column 8 of Table II B, application no. 3, and being depicted in the same respective line as said B3098, and preferably the activity is increased non-targeted.

Accordingly, in one embodiment, the molecule which activity is to be increased in the process of the invention is the gene product with an activity as a “b3098-protein”, preferably it is the molecule of section (a) or (b) of this paragraph.

In particular, it was observed that in plants, especially in Arabidopsis thaliana, increasing or generating the activity of a gene product non-targeted with the activity of a “b3098-protein”, preferably being encoded by a gene comprising the nucleic acid sequence SEQ ID NO. 82459, preferably the coding region thereof, conferred the production of or the increase in alanine compared with the wild type control.

The nucleic acid sequence of B3098 from Escherichia coli, e.g. as shown in the respective line in column 5 of Table I, application no. 3, has been published in B. And the activity of the gene product thereof is the activity of b3098-protein.

Accordingly, in one embodiment, the process of the present invention for producing glycine in a non-human organism, like a microorganism or a plant or a part thereof, comprises increasing or generating the activity of a gene product with the activity of a gene product conferring the activity of “b3098-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3098, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3098, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3098, or a functional equivalent or a homolog thereof as depicted in column 8 of Table II, application no. 3, preferably a homolog or functional equivalent as depicted in column 8 of Table II B, application no. 3, and being depicted in the same respective line as said B3098, and preferably the activity is increased non-targeted.

Accordingly, in one embodiment, the molecule which activity is to be increased in the process of the invention is the gene product with an activity as a “b3098-protein”, preferably it is the molecule of section (a) or (b) of this paragraph.

In particular, it was observed that in plants, especially in Arabidopsis thaliana, increasing or generating the activity of a gene product non-targeted with the activity of a “b3098-protein”, preferably being encoded by a gene comprising the nucleic acid sequence SEQ ID NO. 82459, preferably the coding region thereof, conferred the production of or the increase in glycine compared with the wild type control.

The nucleic acid sequence of B3121 from Escherichia coli, e.g. as shown in the respective line in column 5 of Table I, application no. 3, has been published in B. And the activity of the gene product thereof is the activity of b3121-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “b3121-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3121, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3121, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3121, or a functional equivalent or a homolog thereof as depicted in column 8 of Table II, application no. 3, preferably a homolog or functional equivalent as depicted in column 8 of Table II B, application no. 3, and being depicted in the same respective line as said B3121, and preferably the activity is increased non-targeted.

Accordingly, in one embodiment, the molecule which activity is to be increased in the process of the invention is the gene product with an activity as a “b3121-protein”, preferably it is the molecule of section (a) or (b) of this paragraph.

In particular, it was observed that in plants, especially in Arabidopsis thaliana, increasing or generating the activity of a gene product non-targeted with the activity of a “b3121-protein”, preferably being encoded by a gene comprising the nucleic acid sequence SEQ ID NO. 42471, preferably the coding region thereof, conferred the production of or the increase in tyrosine compared with the wild type control.

The nucleic acid sequence of B3221 from Escherichia coli, e.g. as shown in the respective line in column 5 of Table I, application no. 3, has been published in B. And the activity of the gene product thereof is the activity of b3221-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “b3221-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3221, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3221, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3221, or a functional equivalent or a homolog thereof as depicted in column 8 of Table II, application no. 3, preferably a homolog or functional equivalent as depicted in column 8 of Table II B, application no. 3, and being depicted in the same respective line as said B3221, and preferably the activity is increased plastidic.

Accordingly, in one embodiment, the molecule which activity is to be increased in the process of the invention is the gene product with an activity as a “b3221-protein”, preferably it is the molecule of section (a) or (b) of this paragraph.

In particular, it was observed that in plants, especially in Arabidopsis thaliana, increasing or generating the activity of a gene product plastidic with the activity of a “b3221-protein”, preferably being encoded by a gene comprising the nucleic acid sequence SEQ ID NO. 82510, preferably the coding region thereof, conferred the production of or the increase in tyrosine compared with the wild type control.

The nucleic acid sequence of B3246 from Escherichia coli, e.g. as shown in the respective line in column 5 of Table I, application no. 3, has been published in B. And the activity of the gene product thereof is the activity of b3246-protein.

Accordingly, in one embodiment, the process of the present invention for producing serine in a non-human organism, like a microorganism or a plant or a part thereof, comprises increasing or generating the activity of a gene product with the activity of a gene product conferring the activity of “b3246-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3246, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3246, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3246, or a functional equivalent or a homolog thereof as depicted in column 8 of Table II, application no. 3, preferably a homolog or functional equivalent as depicted in column 8 of Table II B, application no. 3, and being depicted in the same respective line as said B3246, and preferably the activity is increased non-targeted.

Accordingly, in one embodiment, the molecule which activity is to be increased in the process of the invention is the gene product with an activity as a “b3246-protein”, preferably it is the molecule of section (a) or (b) of this paragraph.

In particular, it was observed that in plants, especially in Arabidopsis thaliana, increasing or generating the activity of a gene product non-targeted with the activity of a “b3246-protein”, preferably being encoded by a gene comprising the nucleic acid sequence SEQ ID NO. 9470, preferably the coding region thereof, conferred the production of or the increase in serine compared with the wild type control.

The nucleic acid sequence of B3246 from Escherichia coli, e.g. as shown in the respective line in column 5 of Table I, application no. 3, has been published in B. And the activity of the gene product thereof is the activity of b3246-protein.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “b3246-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3246, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3246, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3246, or a functional equivalent or a homolog thereof as depicted in column 8 of Table II, application no. 3, preferably a homolog or functional equivalent as depicted in column 8 of Table II B, application no. 3, and being depicted in the same respective line as said B3246, and preferably the activity is increased non-targeted.

Accordingly, in one embodiment, the molecule which activity is to be increased in the process of the invention is the gene product with an activity as a “b3246-protein”, preferably it is the molecule of section (a) or (b) of this paragraph.

In particular, it was observed that in plants, especially in Arabidopsis thaliana, increasing or generating the activity of a gene product non-targeted with the activity of a “b3246-protein”, preferably being encoded by a gene comprising the nucleic acid sequence SEQ ID NO. 9470, preferably the coding region thereof, conferred the production of or the increase in 5-oxoproline compared with the wild type control.

The nucleic acid sequence of B3256 from Escherichia coli, e.g. as shown in the respective line in column 5 of Table I, application no. 3, has been published in B. And the activity of the gene product thereof is the activity of acetyl CoA carboxylase.

Accordingly, in one embodiment, the process of the present invention for producing asparagine in a non-human organism, like a microorganism or a plant or a part thereof, comprises increasing or generating the activity of a gene product with the activity of a gene product conferring the activity of “acetyl CoA carboxylase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3256, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3256, or a functional equivalent or a homolog thereof as depicted in column 8 of Table II, application no. 3, preferably a homolog or functional equivalent as depicted in column 8 of Table II B, application no. 3, and being depicted in the same respective line as said B3256, and preferably the activity is increased non-targeted.

Accordingly, in one embodiment, the molecule which activity is to be increased in the process of the invention is the gene product with an activity as a “acetyl CoA carboxylase”, preferably it is the molecule of section (a) or (b) of this paragraph.

In particular, it was observed that in plants, especially in Arabidopsis thaliana, increasing or generating the activity of a gene product non-targeted with the activity of a “acetyl CoA carboxylase”, preferably being encoded by a gene comprising the nucleic acid sequence SEQ ID NO. 9492, preferably the coding region thereof, conferred the production of or the increase in asparagine compared with the wild type control.

The nucleic acid sequence of B3256 from Escherichia coli, e.g. as shown in the respective line in column 5 of Table I, application no. 3, has been published in B. And the activity of the gene product thereof is the activity of acetyl CoA carboxylase.

Accordingly, in one embodiment, the process of the present invention for producing glycine in a non-human organism, like a microorganism or a plant or a part thereof, comprises increasing or generating the activity of a gene product with the activity of a gene product conferring the activity of “acetyl CoA carboxylase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3256, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3256, or a functional equivalent or a homolog thereof as depicted in column 8 of Table II, application no. 3, preferably a homolog or functional equivalent as depicted in column 8 of Table II B, application no. 3, and being depicted in the same respective line as said B3256, and preferably the activity is increased non-targeted.

Accordingly, in one embodiment, the molecule which activity is to be increased in the process of the invention is the gene product with an activity as a “acetyl CoA carboxylase”, preferably it is the molecule of section (a) or (b) of this paragraph.

In particular, it was observed that in plants, especially in Arabidopsis thaliana, increasing or generating the activity of a gene product non-targeted with the activity of a “acetyl CoA carboxylase”, preferably being encoded by a gene comprising the nucleic acid sequence SEQ ID NO. 9492, preferably the coding region thereof, conferred the production of or the increase in glycine compared with the wild type control.

The nucleic acid sequence of B3256 from Escherichia coli, e.g. as shown in the respective line in column 5 of Table I, application no. 3, has been published in B. And the activity of the gene product thereof is the activity of acetyl CoA carboxylase.

Accordingly, in one embodiment, the process of the present invention for producing serine in a non-human organism, like a microorganism or a plant or a part thereof, comprises increasing or generating the activity of a gene product with the activity of a gene product conferring the activity of “acetyl CoA carboxylase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3256, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3256, or a functional equivalent or a homolog thereof as depicted in column 8 of Table II, application no. 3, preferably a homolog or functional equivalent as depicted in column 8 of Table II B, application no. 3, and being depicted in the same respective line as said B3256, and preferably the activity is increased non-targeted.

Accordingly, in one embodiment, the molecule which activity is to be increased in the process of the invention is the gene product with an activity as a “acetyl CoA carboxylase”, preferably it is the molecule of section (a) or (b) of this paragraph.

In particular, it was observed that in plants, especially in Arabidopsis thaliana, increasing or generating the activity of a gene product non-targeted with the activity of a “acetyl CoA carboxylase”, preferably being encoded by a gene comprising the nucleic acid sequence SEQ ID NO. 9492, preferably the coding region thereof, conferred the production of or the increase in serine compared with the wild type control.

The nucleic acid sequence of B3256 from Escherichia coli, e.g. as shown in the respective line in column 5 of Table I, application no. 3, has been published in B. And the activity of the gene product thereof is the activity of acetyl CoA carboxylase.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “acetyl CoA carboxylase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3256, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3256, or a functional equivalent or a homolog thereof as depicted in column 8 of Table II, application no. 3, preferably a homolog or functional equivalent as depicted in column 8 of Table II B, application no. 3, and being depicted in the same respective line as said B3256, and preferably the activity is increased non-targeted.

Accordingly, in one embodiment, the molecule which activity is to be increased in the process of the invention is the gene product with an activity as a “acetyl CoA carboxylase”, preferably it is the molecule of section (a) or (b) of this paragraph.

In particular, it was observed that in plants, especially in Arabidopsis thaliana, increasing or generating the activity of a gene product non-targeted with the activity of a “acetyl CoA carboxylase”, preferably being encoded by a gene comprising the nucleic acid sequence SEQ ID NO. 9492, preferably the coding region thereof, conferred the production of or the increase in 5-oxoproline compared with the wild type control.

The nucleic acid sequence of B3262 from Escherichia coli, e.g. as shown in the respective line in column 5 of Table I, application no. 3, has been published in B. And the activity of the gene product thereof is the activity of methyltransferase.

Accordingly, in one embodiment, the process of the present invention for producing alanine in a non-human organism, like a microorganism or a plant or a part thereof, comprises increasing or generating the activity of a gene product with the activity of a gene product conferring the activity of “methyltransferase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3262, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3262, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3262, or a functional equivalent or a homolog thereof as depicted in column 8 of Table II, application no. 3, preferably a homolog or functional equivalent as depicted in column 8 of Table II B, application no. 3, and being depicted in the same respective line as said B3262, and preferably the activity is increased non-targeted.

Accordingly, in one embodiment, the molecule which activity is to be increased in the process of the invention is the gene product with an activity as a “methyltransferase”, preferably it is the molecule of section (a) or (b) of this paragraph.

In particular, it was observed that in plants, especially in Arabidopsis thaliana, increasing or generating the activity of a gene product non-targeted with the activity of a “methyltransferase”, preferably being encoded by a gene comprising the nucleic acid sequence SEQ ID NO. 42502, preferably the coding region thereof, conferred the production of or the increase in alanine compared with the wild type control.

The nucleic acid sequence of B3262 from Escherichia coli, e.g. as shown in the respective line in column 5 of Table I, application no. 3, has been published in B. And the activity of the gene product thereof is the activity of methyltransferase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “methyltransferase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3262, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3262, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3262, or a functional equivalent or a homolog thereof as depicted in column 8 of Table II, application no. 3, preferably a homolog or functional equivalent as depicted in column 8 of Table II B, application no. 3, and being depicted in the same respective line as said B3262, and preferably the activity is increased non-targeted.

Accordingly, in one embodiment, the molecule which activity is to be increased in the process of the invention is the gene product with an activity as a “methyltransferase”, preferably it is the molecule of section (a) or (b) of this paragraph.

In particular, it was observed that in plants, especially in Arabidopsis thaliana, increasing or generating the activity of a gene product non-targeted with the activity of a “methyltransferase”, preferably being encoded by a gene comprising the nucleic acid sequence SEQ ID NO. 42502, preferably the coding region thereof, conferred the production of or the increase in phenylalanine compared with the wild type control.

The nucleic acid sequence of B3262 from Escherichia coli, e.g. as shown in the respective line in column 5 of Table I, application no. 3, has been published in B. And the activity of the gene product thereof is the activity of methyltransferase.

Accordingly, in one embodiment, the process of the present invention for producing serine in a non-human organism, like a microorganism or a plant or a part thereof, comprises increasing or generating the activity of a gene product with the activity of a gene product conferring the activity of “methyltransferase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3262, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3262, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3262, or a functional equivalent or a homolog thereof as depicted in column 8 of Table II, application no. 3, preferably a homolog or functional equivalent as depicted in column 8 of Table II B, application no. 3, and being depicted in the same respective line as said B3262, and preferably the activity is increased non-targeted.

Accordingly, in one embodiment, the molecule which activity is to be increased in the process of the invention is the gene product with an activity as a “methyltransferase”, preferably it is the molecule of section (a) or (b) of this paragraph.

In particular, it was observed that in plants, especially in Arabidopsis thaliana, increasing or generating the activity of a gene product non-targeted with the activity of a “methyltransferase”, preferably being encoded by a gene comprising the nucleic acid sequence SEQ ID NO. 42502, preferably the coding region thereof, conferred the production of or the increase in serine compared with the wild type control.

The nucleic acid sequence of B3262 from Escherichia coli, e.g. as shown in the respective line in column 5 of Table I, application no. 3, has been published in B. And the activity of the gene product thereof is the activity of methyltransferase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “methyltransferase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3262, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3262, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3262, or a functional equivalent or a homolog thereof as depicted in column 8 of Table II, application no. 3, preferably a homolog or functional equivalent as depicted in column 8 of Table II B, application no. 3, and being depicted in the same respective line as said B3262, and preferably the activity is increased non-targeted.

Accordingly, in one embodiment, the molecule which activity is to be increased in the process of the invention is the gene product with an activity as a “methyltransferase”, preferably it is the molecule of section (a) or (b) of this paragraph.

In particular, it was observed that in plants, especially in Arabidopsis thaliana, increasing or generating the activity of a gene product non-targeted with the activity of a “methyltransferase”, preferably being encoded by a gene comprising the nucleic acid sequence SEQ ID NO. 42502, preferably the coding region thereof, conferred the production of or the increase in tyrosine compared with the wild type control.

The nucleic acid sequence of B3262 from Escherichia coli, e.g. as shown in the respective line in column 5 of Table I, application no. 3, has been published in B. And the activity of the gene product thereof is the activity of methyltransferase.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “methyltransferase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3262, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3262, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3262, or a functional equivalent or a homolog thereof as depicted in column 8 of Table II, application no. 3, preferably a homolog or functional equivalent as depicted in column 8 of Table II B, application no. 3, and being depicted in the same respective line as said B3262, and preferably the activity is increased non-targeted.

Accordingly, in one embodiment, the molecule which activity is to be increased in the process of the invention is the gene product with an activity as a “methyltransferase”, preferably it is the molecule of section (a) or (b) of this paragraph.

In particular, it was observed that in plants, especially in Arabidopsis thaliana, increasing or generating the activity of a gene product non-targeted with the activity of a “methyltransferase”, preferably being encoded by a gene comprising the nucleic acid sequence SEQ ID NO. 42502, preferably the coding region thereof, conferred the production of or the increase in 5-oxoproline compared with the wild type control.

The nucleic acid sequence of B3346 from Escherichia coli, e.g. as shown in the respective line in column 5 of Table I, application no. 3, has been published in B. And the activity of the gene product thereof is the activity of b3346-protein.

Accordingly, in one embodiment, the process of the present invention for producing alanine in a non-human organism, like a microorganism or a plant or a part thereof, comprises increasing or generating the activity of a gene product with the activity of a gene product conferring the activity of “b3346-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3346, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3346, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3346, or a functional equivalent or a homolog thereof as depicted in column 8 of Table II, application no. 3, preferably a homolog or functional equivalent as depicted in column 8 of Table II B, application no. 3, and being depicted in the same respective line as said B3346, and preferably the activity is increased non-targeted.

Accordingly, in one embodiment, the molecule which activity is to be increased in the process of the invention is the gene product with an activity as a “b3346-protein”, preferably it is the molecule of section (a) or (b) of this paragraph.

In particular, it was observed that in plants, especially in Arabidopsis thaliana, increasing or generating the activity of a gene product non-targeted with the activity of a “b3346-protein”, preferably being encoded by a gene comprising the nucleic acid sequence SEQ ID NO. 10104, preferably the coding region thereof, conferred the production of or the increase in alanine compared with the wild type control.

The nucleic acid sequence of B3352 from Escherichia coli, e.g. as shown in the respective line in column 5 of Table I, application no. 3, has been published in B. And the activity of the gene product thereof is the activity of ABC transporter ATP-binding protein.

Accordingly, in one embodiment, the process of the present invention for producing alanine in a non-human organism, like a microorganism or a plant or a part thereof, comprises increasing or generating the activity of a gene product with the activity of a gene product conferring the activity of “ABC transporter ATP-binding protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3352, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3352, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3352, or a functional equivalent or a homolog thereof as depicted in column 8 of Table II, application no. 3, preferably a homolog or functional equivalent as depicted in column 8 of Table II B, application no. 3, and being depicted in the same respective line as said B3352, and preferably the activity is increased non-targeted.

Accordingly, in one embodiment, the molecule which activity is to be increased in the process of the invention is the gene product with an activity as a “ABC transporter ATP-binding protein”, preferably it is the molecule of section (a) or (b) of this paragraph.

In particular, it was observed that in plants, especially in Arabidopsis thaliana, increasing or generating the activity of a gene product non-targeted with the activity of a “ABC transporter ATP-binding protein”, preferably being encoded by a gene comprising the nucleic acid sequence SEQ ID NO. 82572, preferably the coding region thereof, conferred the production of or the increase in alanine compared with the wild type control.

The nucleic acid sequence of B3352 from Escherichia coli, e.g. as shown in the respective line in column 5 of Table I, application no. 3, has been published in B. And the activity of the gene product thereof is the activity of ABC transporter ATP-binding protein.

Accordingly, in one embodiment, the process of the present invention for producing glycine in a non-human organism, like a microorganism or a plant or a part thereof, comprises increasing or generating the activity of a gene product with the activity of a gene product conferring the activity of “ABC transporter ATP-binding protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3352, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3352, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3352, or a functional equivalent or a homolog thereof as depicted in column 8 of Table II, application no. 3, preferably a homolog or functional equivalent as depicted in column 8 of Table II B, application no. 3, and being depicted in the same respective line as said B3352, and preferably the activity is increased non-targeted.

Accordingly, in one embodiment, the molecule which activity is to be increased in the process of the invention is the gene product with an activity as a “ABC transporter ATP-binding protein”, preferably it is the molecule of section (a) or (b) of this paragraph.

In particular, it was observed that in plants, especially in Arabidopsis thaliana, increasing or generating the activity of a gene product non-targeted with the activity of a “ABC transporter ATP-binding protein”, preferably being encoded by a gene comprising the nucleic acid sequence SEQ ID NO. 82572, preferably the coding region thereof, conferred the production of or the increase in glycine compared with the wild type control.

The nucleic acid sequence of B3392 from Escherichia coli, e.g. as shown in the respective line in column 5 of Table I, application no. 3, has been published in B. And the activity of the gene product thereof is the activity of b3392-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “b3392-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3392, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3392, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3392, or a functional equivalent or a homolog thereof as depicted in column 8 of Table II, application no. 3, preferably a homolog or functional equivalent as depicted in column 8 of Table II B, application no. 3, and being depicted in the same respective line as said B3392, and preferably the activity is increased plastidic.

Accordingly, in one embodiment, the molecule which activity is to be increased in the process of the invention is the gene product with an activity as a “b3392-protein”, preferably it is the molecule of section (a) or (b) of this paragraph.

In particular, it was observed that in plants, especially in Arabidopsis thaliana, increasing or generating the activity of a gene product plastidic with the activity of a “b3392-protein”, preferably being encoded by a gene comprising the nucleic acid sequence SEQ ID NO. 82846, preferably the coding region thereof, conferred the production of or the increase in aspartate compared with the wild type control.

The nucleic acid sequence of B3403 from Escherichia coli, e.g. as shown in the respective line in column 5 of Table I, application no. 3, has been published in B. And the activity of the gene product thereof is the activity of phosphoenolpyruvate carboxykinase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “phosphoenolpyruvate carboxykinase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3403, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3403, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3403, or a functional equivalent or a homolog thereof as depicted in column 8 of Table II, application no. 3, preferably a homolog or functional equivalent as depicted in column 8 of Table II B, application no. 3, and being depicted in the same respective line as said B3403, and preferably the activity is increased non-targeted.

Accordingly, in one embodiment, the molecule which activity is to be increased in the process of the invention is the gene product with an activity as a “phosphoenolpyruvate carboxykinase”, preferably it is the molecule of section (a) or (b) of this paragraph.

In particular, it was observed that in plants, especially in Arabidopsis thaliana, increasing or generating the activity of a gene product non-targeted with the activity of a “phosphoenolpyruvate carboxykinase”, preferably being encoded by a gene comprising the nucleic acid sequence SEQ ID NO. 82866, preferably the coding region thereof, conferred the production of or the increase in phenylalanine compared with the wild type control.

The nucleic acid sequence of B3403 from Escherichia coli, e.g. as shown in the respective line in column 5 of Table I, application no. 3, has been published in B. And the activity of the gene product thereof is the activity of phosphoenolpyruvate carboxykinase.

Accordingly, in one embodiment, the process of the present invention for producing serine in a non-human organism, like a microorganism or a plant or a part thereof, comprises increasing or generating the activity of a gene product with the activity of a gene product conferring the activity of “phosphoenolpyruvate carboxykinase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3403, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3403, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3403, or a functional equivalent or a homolog thereof as depicted in column 8 of Table II, application no. 3, preferably a homolog or functional equivalent as depicted in column 8 of Table II B, application no. 3, and being depicted in the same respective line as said B3403, and preferably the activity is increased non-targeted.

Accordingly, in one embodiment, the molecule which activity is to be increased in the process of the invention is the gene product with an activity as a “phosphoenolpyruvate carboxykinase”, preferably it is the molecule of section (a) or (b) of this paragraph.

In particular, it was observed that in plants, especially in Arabidopsis thaliana, increasing or generating the activity of a gene product non-targeted with the activity of a “phosphoenolpyruvate carboxykinase”, preferably being encoded by a gene comprising the nucleic acid sequence SEQ ID NO. 82866, preferably the coding region thereof, conferred the production of or the increase in serine compared with the wild type control.

The nucleic acid sequence of B3410 from Escherichia coli, e.g. as shown in the respective line in column 5 of Table I, application no. 3, has been published in B. And the activity of the gene product thereof is the activity of b3410-protein.

Accordingly, in one embodiment, the process of the present invention for producing alanine in a non-human organism, like a microorganism or a plant or a part thereof, comprises increasing or generating the activity of a gene product with the activity of a gene product conferring the activity of “b3410-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3410, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3410, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3410, or a functional equivalent or a homolog thereof as depicted in column 8 of Table II, application no. 3, preferably a homolog or functional equivalent as depicted in column 8 of Table II B, application no. 3, and being depicted in the same respective line as said B3410, and preferably the activity is increased non-targeted.

Accordingly, in one embodiment, the molecule which activity is to be increased in the process of the invention is the gene product with an activity as a “b3410-protein”, preferably it is the molecule of section (a) or (b) of this paragraph.

In particular, it was observed that in plants, especially in Arabidopsis thaliana, increasing or generating the activity of a gene product non-targeted with the activity of a “b3410-protein”, preferably being encoded by a gene comprising the nucleic acid sequence SEQ ID NO. 42559, preferably the coding region thereof, conferred the production of or the increase in alanine compared with the wild type control.

The nucleic acid sequence of B3509 from Escherichia coli, e.g. as shown in the respective line in column 5 of Table I, application no. 3, has been published in B. And the activity of the gene product thereof is the activity of b3509-protein.

Accordingly, in one embodiment, the process of the present invention for producing serine in a non-human organism, like a microorganism or a plant or a part thereof, comprises increasing or generating the activity of a gene product with the activity of a gene product conferring the activity of “b3509-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3509, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3509, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3509, or a functional equivalent or a homolog thereof as depicted in column 8 of Table II, application no. 3, preferably a homolog or functional equivalent as depicted in column 8 of Table II B, application no. 3, and being depicted in the same respective line as said B3509, and preferably the activity is increased non-targeted.

Accordingly, in one embodiment, the molecule which activity is to be increased in the process of the invention is the gene product with an activity as a “b3509-protein”, preferably it is the molecule of section (a) or (b) of this paragraph.

In particular, it was observed that in plants, especially in Arabidopsis thaliana, increasing or generating the activity of a gene product non-targeted with the activity of a “b3509-protein”, preferably being encoded by a gene comprising the nucleic acid sequence SEQ ID NO. 42592, preferably the coding region thereof, conferred the production of or the increase in serine compared with the wild type control.

The nucleic acid sequence of B3533 from Escherichia coli, e.g. as shown in the respective line in column 5 of Table I, application no. 3, has been published in B. And the activity of the gene product thereof is the activity of cellulose synthase catalytic subunit.

Accordingly, in one embodiment, the process of the present invention for producing aspartate in a non-human organism, like a microorganism or a plant or a part thereof, comprises increasing or generating the activity of a gene product with the activity of a gene product conferring the activity of “cellulose synthase catalytic subunit”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3533, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3533, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3533, or a functional equivalent or a homolog thereof as depicted in column 8 of Table II, application no. 3, preferably a homolog or functional equivalent as depicted in column 8 of Table II B, application no. 3, and being depicted in the same respective line as said B3533, and preferably the activity is increased non-targeted.

Accordingly, in one embodiment, the molecule which activity is to be increased in the process of the invention is the gene product with an activity as a “cellulose synthase catalytic subunit”, preferably it is the molecule of section (a) or (b) of this paragraph.

In particular, it was observed that in plants, especially in Arabidopsis thaliana, increasing or generating the activity of a gene product non-targeted with the activity of a “cellulose synthase catalytic subunit”, preferably being encoded by a gene comprising the nucleic acid sequence SEQ ID NO. 83153, preferably the coding region thereof, conferred the production of or the increase in aspartate compared with the wild type control.

The nucleic acid sequence of B3607 from Escherichia coli, e.g. as shown in the respective line in column 5 of Table I, application no. 3, has been published in B. And the activity of the gene product thereof is the activity of serine acetyltransferase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “serine acetyltransferase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3607, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3607, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3607, or a functional equivalent or a homolog thereof as depicted in column 8 of Table II, application no. 3, preferably a homolog or functional equivalent as depicted in column 8 of Table II B, application no. 3, and being depicted in the same respective line as said B3607, and preferably the activity is increased plastidic.

Accordingly, in one embodiment, the molecule which activity is to be increased in the process of the invention is the gene product with an activity as a “serine acetyltransferase”, preferably it is the molecule of section (a) or (b) of this paragraph.

In particular, it was observed that in plants, especially in Arabidopsis thaliana, increasing or generating the activity of a gene product plastidic with the activity of a “serine acetyltransferase”, preferably being encoded by a gene comprising the nucleic acid sequence SEQ ID NO. 83213, preferably the coding region thereof, conferred the production of or the increase in phenylalanine compared with the wild type control.

The nucleic acid sequence of B3607 from Escherichia coli, e.g. as shown in the respective line in column 5 of Table I, application no. 3, has been published in B. And the activity of the gene product thereof is the activity of serine acetyltransferase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “serine acetyltransferase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3607, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3607, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3607, or a functional equivalent or a homolog thereof as depicted in column 8 of Table II, application no. 3, preferably a homolog or functional equivalent as depicted in column 8 of Table II B, application no. 3, and being depicted in the same respective line as said B3607, and preferably the activity is increased plastidic.

Accordingly, in one embodiment, the molecule which activity is to be increased in the process of the invention is the gene product with an activity as a “serine acetyltransferase”, preferably it is the molecule of section (a) or (b) of this paragraph.

In particular, it was observed that in plants, especially in Arabidopsis thaliana, increasing or generating the activity of a gene product plastidic with the activity of a “serine acetyltransferase”, preferably being encoded by a gene comprising the nucleic acid sequence SEQ ID NO. 83213, preferably the coding region thereof, conferred the production of or the increase in tyrosine compared with the wild type control.

The nucleic acid sequence of B3634 from Escherichia coli, e.g. as shown in the respective line in column 5 of Table I, application no. 3, has been published in B. And the activity of the gene product thereof is the activity of phosphopantetheine adenylyltransferase.

Accordingly, in one embodiment, the process of the present invention for producing alanine in a non-human organism, like a microorganism or a plant or a part thereof, comprises increasing or generating the activity of a gene product with the activity of a gene product conferring the activity of “phosphopantetheine adenylyltransferase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3634, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3634, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3634, or a functional equivalent or a homolog thereof as depicted in column 8 of Table II, application no. 3, preferably a homolog or functional equivalent as depicted in column 8 of Table II B, application no. 3, and being depicted in the same respective line as said B3634, and preferably the activity is increased non-targeted.

Accordingly, in one embodiment, the molecule which activity is to be increased in the process of the invention is the gene product with an activity as a “phosphopantetheine adenylyltransferase”, preferably it is the molecule of section (a) or (b) of this paragraph.

In particular, it was observed that in plants, especially in Arabidopsis thaliana, increasing or generating the activity of a gene product non-targeted with the activity of a “phosphopantetheine adenylyltransferase”, preferably being encoded by a gene comprising the nucleic acid sequence SEQ ID NO. 42931, preferably the coding region thereof, conferred the production of or the increase in alanine compared with the wild type control.

The nucleic acid sequence of B3634 from Escherichia coli, e.g. as shown in the respective line in column 5 of Table I, application no. 3, has been published in B. And the activity of the gene product thereof is the activity of phosphopantetheine adenylyltransferase.

Accordingly, in one embodiment, the process of the present invention for producing asparagine in a non-human organism, like a microorganism or a plant or a part thereof, comprises increasing or generating the activity of a gene product with the activity of a gene product conferring the activity of “phosphopantetheine adenylyltransferase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3634, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3634, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3634, or a functional equivalent or a homolog thereof as depicted in column 8 of Table II, application no. 3, preferably a homolog or functional equivalent as depicted in column 8 of Table II B, application no. 3, and being depicted in the same respective line as said B3634, and preferably the activity is increased non-targeted.

Accordingly, in one embodiment, the molecule which activity is to be increased in the process of the invention is the gene product with an activity as a “phosphopantetheine adenylyltransferase”, preferably it is the molecule of section (a) or (b) of this paragraph.

In particular, it was observed that in plants, especially in Arabidopsis thaliana, increasing or generating the activity of a gene product non-targeted with the activity of a “phosphopantetheine adenylyltransferase”, preferably being encoded by a gene comprising the nucleic acid sequence SEQ ID NO. 42931, preferably the coding region thereof, conferred the production of or the increase in asparagine compared with the wild type control.

The nucleic acid sequence of B3634 from Escherichia coli, e.g. as shown in the respective line in column 5 of Table I, application no. 3, has been published in B. And the activity of the gene product thereof is the activity of phosphopantetheine adenylyltransferase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “phosphopantetheine adenylyltransferase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3634, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3634, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3634, or a functional equivalent or a homolog thereof as depicted in column 8 of Table II, application no. 3, preferably a homolog or functional equivalent as depicted in column 8 of Table II B, application no. 3, and being depicted in the same respective line as said B3634, and preferably the activity is increased non-targeted.

Accordingly, in one embodiment, the molecule which activity is to be increased in the process of the invention is the gene product with an activity as a “phosphopantetheine adenylyltransferase”, preferably it is the molecule of section (a) or (b) of this paragraph.

In particular, it was observed that in plants, especially in Arabidopsis thaliana, increasing or generating the activity of a gene product non-targeted with the activity of a “phosphopantetheine adenylyltransferase”, preferably being encoded by a gene comprising the nucleic acid sequence SEQ ID NO. 42931, preferably the coding region thereof, conferred the production of or the increase in phenylalanine compared with the wild type control.

The nucleic acid sequence of B3646 from Escherichia coli, e.g. as shown in the respective line in column 5 of Table I, application no. 3, has been published in B. And the activity of the gene product thereof is the activity of b3646-protein.

Accordingly, in one embodiment, the process of the present invention for producing glycine in a non-human organism, like a microorganism or a plant or a part thereof, comprises increasing or generating the activity of a gene product with the activity of a gene product conferring the activity of “b3646-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3646, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3646, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3646, or a functional equivalent or a homolog thereof as depicted in column 8 of Table II, application no. 3, preferably a homolog or functional equivalent as depicted in column 8 of Table II B, application no. 3, and being depicted in the same respective line as said B3646, and preferably the activity is increased non-targeted.

Accordingly, in one embodiment, the molecule which activity is to be increased in the process of the invention is the gene product with an activity as a “b3646-protein”, preferably it is the molecule of section (a) or (b) of this paragraph.

In particular, it was observed that in plants, especially in Arabidopsis thaliana, increasing or generating the activity of a gene product non-targeted with the activity of a “b3646-protein”, preferably being encoded by a gene comprising the nucleic acid sequence SEQ ID NO. 83429, preferably the coding region thereof, conferred the production of or the increase in glycine compared with the wild type control.

The nucleic acid sequence of B3670 from Escherichia coli, e.g. as shown in the respective line in column 5 of Table I, application no. 3, has been published in B. And the activity of the gene product thereof is the activity of acetolactate synthase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “acetolactate synthase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3670, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3670, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3670, or a functional equivalent or a homolog thereof as depicted in column 8 of Table II, application no. 3, preferably a homolog or functional equivalent as depicted in column 8 of Table II B, application no. 3, and being depicted in the same respective line as said B3670, and preferably the activity is increased plastidic.

Accordingly, in one embodiment, the molecule which activity is to be increased in the process of the invention is the gene product with an activity as a “acetolactate synthase”, preferably it is the molecule of section (a) or (b) of this paragraph.

In particular, it was observed that in plants, especially in Arabidopsis thaliana, increasing or generating the activity of a gene product plastidic with the activity of a “acetolactate synthase”, preferably being encoded by a gene comprising the nucleic acid sequence SEQ ID NO. 83629, preferably the coding region thereof, conferred the production of or the increase in tyrosine compared with the wild type control.

The nucleic acid sequence of B3744 from Escherichia coli, e.g. as shown in the respective line in column 5 of Table I, application no. 3, has been published in B. And the activity of the gene product thereof is the activity of aspartate-ammonia ligase.

Accordingly, in one embodiment, the process of the present invention for producing asparagine in a non-human organism, like a microorganism or a plant or a part thereof, comprises increasing or generating the activity of a gene product with the activity of a gene product conferring the activity of “aspartate-ammonia ligase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3744, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3744, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3744, or a functional equivalent or a homolog thereof as depicted in column 8 of Table II, application no. 3, preferably a homolog or functional equivalent as depicted in column 8 of Table II B, application no. 3, and being depicted in the same respective line as said B3744, and preferably the activity is increased plastidic.

Accordingly, in one embodiment, the molecule which activity is to be increased in the process of the invention is the gene product with an activity as a “aspartate-ammonia ligase”, preferably it is the molecule of section (a) or (b) of this paragraph.

In particular, it was observed that in plants, especially in Arabidopsis thaliana, increasing or generating the activity of a gene product plastidic with the activity of a “aspartate-ammonia ligase”, preferably being encoded by a gene comprising the nucleic acid sequence SEQ ID NO. 83657, preferably the coding region thereof, conferred the production of or the increase in asparagine compared with the wild type control.

The nucleic acid sequence of B3771 from Escherichia coli, e.g. as shown in the respective line in column 5 of Table I, application no. 3, has been published in B. And the activity of the gene product thereof is the activity of dihydroxyacid dehydratase.

Accordingly, in one embodiment, the process of the present invention for producing glycine in a non-human organism, like a microorganism or a plant or a part thereof, comprises increasing or generating the activity of a gene product with the activity of a gene product conferring the activity of “dihydroxyacid dehydratase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3771, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3771, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3771, or a functional equivalent or a homolog thereof as depicted in column 8 of Table II, application no. 3, preferably a homolog or functional equivalent as depicted in column 8 of Table II B, application no. 3, and being depicted in the same respective line as said B3771, and preferably the activity is increased non-targeted.

Accordingly, in one embodiment, the molecule which activity is to be increased in the process of the invention is the gene product with an activity as a “dihydroxyacid dehydratase”, preferably it is the molecule of section (a) or (b) of this paragraph.

In particular, it was observed that in plants, especially in Arabidopsis thaliana, increasing or generating the activity of a gene product non-targeted with the activity of a “dihydroxyacid dehydratase”, preferably being encoded by a gene comprising the nucleic acid sequence SEQ ID NO. 10252, preferably the coding region thereof, conferred the production of or the increase in glycine compared with the wild type control.

The nucleic acid sequence of B3771 from Escherichia coli, e.g. as shown in the respective line in column 5 of Table I, application no. 3, has been published in B. And the activity of the gene product thereof is the activity of dihydroxyacid dehydratase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “dihydroxyacid dehydratase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3771, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3771, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3771, or a functional equivalent or a homolog thereof as depicted in column 8 of Table II, application no. 3, preferably a homolog or functional equivalent as depicted in column 8 of Table II B, application no. 3, and being depicted in the same respective line as said B3771, and preferably the activity is increased non-targeted.

Accordingly, in one embodiment, the molecule which activity is to be increased in the process of the invention is the gene product with an activity as a “dihydroxyacid dehydratase”, preferably it is the molecule of section (a) or (b) of this paragraph.

In particular, it was observed that in plants, especially in Arabidopsis thaliana, increasing or generating the activity of a gene product non-targeted with the activity of a “dihydroxyacid dehydratase”, preferably being encoded by a gene comprising the nucleic acid sequence SEQ ID NO. 10252, preferably the coding region thereof, conferred the production of or the increase in phenylalanine compared with the wild type control.

The nucleic acid sequence of B3791 from Escherichia coli, e.g. as shown in the respective line in column 5 of Table I, application no. 3, has been published in B. And the activity of the gene product thereof is the activity of b3791-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “b3791-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3791, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3791, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3791, or a functional equivalent or a homolog thereof as depicted in column 8 of Table II, application no. 3, preferably a homolog or functional equivalent as depicted in column 8 of Table II B, application no. 3, and being depicted in the same respective line as said B3791, and preferably the activity is increased non-targeted.

Accordingly, in one embodiment, the molecule which activity is to be increased in the process of the invention is the gene product with an activity as a “b3791-protein”, preferably it is the molecule of section (a) or (b) of this paragraph.

In particular, it was observed that in plants, especially in Arabidopsis thaliana, increasing or generating the activity of a gene product non-targeted with the activity of a “b3791-protein”, preferably being encoded by a gene comprising the nucleic acid sequence SEQ ID NO. 83726, preferably the coding region thereof, conferred the production of or the increase in aspartate compared with the wild type control.

The nucleic acid sequence of B3791 from Escherichia coli, e.g. as shown in the respective line in column 5 of Table I, application no. 3, has been published in B. And the activity of the gene product thereof is the activity of b3791-protein.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “b3791-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3791, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3791, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3791, or a functional equivalent or a homolog thereof as depicted in column 8 of Table II, application no. 3, preferably a homolog or functional equivalent as depicted in column 8 of Table II B, application no. 3, and being depicted in the same respective line as said B3791, and preferably the activity is increased non-targeted.

Accordingly, in one embodiment, the molecule which activity is to be increased in the process of the invention is the gene product with an activity as a “b3791-protein”, preferably it is the molecule of section (a) or (b) of this paragraph.

In particular, it was observed that in plants, especially in Arabidopsis thaliana, increasing or generating the activity of a gene product non-targeted with the activity of a “b3791-protein”, preferably being encoded by a gene comprising the nucleic acid sequence SEQ ID NO. 83726, preferably the coding region thereof, conferred the production of or the increase in 5-oxoproline compared with the wild type control.

The nucleic acid sequence of B3793 from Escherichia coli, e.g. as shown in the respective line in column 5 of Table I, application no. 3, has been published in B. And the activity of the gene product thereof is the activity of enterobacterial common antigen polymerase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “enterobacterial common antigen polymerase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3793, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3793, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3793, or a functional equivalent or a homolog thereof as depicted in column 8 of Table II, application no. 3, preferably a homolog or functional equivalent as depicted in column 8 of Table II B, application no. 3, and being depicted in the same respective line as said B3793, and preferably the activity is increased non-targeted.

Accordingly, in one embodiment, the molecule which activity is to be increased in the process of the invention is the gene product with an activity as a “enterobacterial common antigen polymerase”, preferably it is the molecule of section (a) or (b) of this paragraph.

In particular, it was observed that in plants, especially in Arabidopsis thaliana, increasing or generating the activity of a gene product non-targeted with the activity of a “enterobacterial common antigen polymerase”, preferably being encoded by a gene comprising the nucleic acid sequence SEQ ID NO. 43800, preferably the coding region thereof, conferred the production of or the increase in phenylalanine compared with the wild type control.

The nucleic acid sequence of B3813 from Escherichia coli, e.g. as shown in the respective line in column 5 of Table I, application no. 3, has been published in B. And the activity of the gene product thereof is the activity of DNA helicase II.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “DNA helicase II”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3813, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3813, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3813, or a functional equivalent or a homolog thereof as depicted in column 8 of Table II, application no. 3, preferably a homolog or functional equivalent as depicted in column 8 of Table II B, application no. 3, and being depicted in the same respective line as said B3813, and preferably the activity is increased non-targeted.

Accordingly, in one embodiment, the molecule which activity is to be increased in the process of the invention is the gene product with an activity as a “DNA helicase II”, preferably it is the molecule of section (a) or (b) of this paragraph.

In particular, it was observed that in plants, especially in Arabidopsis thaliana, increasing or generating the activity of a gene product non-targeted with the activity of a “DNA helicase II”, preferably being encoded by a gene comprising the nucleic acid sequence SEQ ID NO. 43839, preferably the coding region thereof, conferred the production of or the increase in phenylalanine compared with the wild type control.

The nucleic acid sequence of B3814 from Escherichia coli, e.g. as shown in the respective line in column 5 of Table I, application no. 3, has been published in B. And the activity of the gene product thereof is the activity of b3814-protein.

Accordingly, in one embodiment, the process of the present invention for producing citrulline in a non-human organism, like a microorganism or a plant or a part thereof, comprises increasing or generating the activity of a gene product with the activity of a gene product conferring the activity of “b3814-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3814, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3814, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3814, or a functional equivalent or a homolog thereof as depicted in column 8 of Table II, application no. 3, preferably a homolog or functional equivalent as depicted in column 8 of Table II B, application no. 3, and being depicted in the same respective line as said B3814, and preferably the activity is increased non-targeted.

Accordingly, in one embodiment, the molecule which activity is to be increased in the process of the invention is the gene product with an activity as a “b3814-protein”, preferably it is the molecule of section (a) or (b) of this paragraph.

In particular, it was observed that in plants, especially in Arabidopsis thaliana, increasing or generating the activity of a gene product non-targeted with the activity of a “b3814-protein”, preferably being encoded by a gene comprising the nucleic acid sequence SEQ ID NO. 44196, preferably the coding region thereof, conferred the production of or the increase in citrulline compared with the wild type control.

The nucleic acid sequence of B3872 from Escherichia coli, e.g. as shown in the respective line in column 5 of Table I, application no. 3, has been published in B. And the activity of the gene product thereof is the activity of transcriptional regulator.

Accordingly, in one embodiment, the process of the present invention for producing alanine in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3872, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3872, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3872, or a functional equivalent or a homolog thereof as depicted in column 8 of Table II, application no. 3, preferably a homolog or functional equivalent as depicted in column 8 of Table II B, application no. 3, and being depicted in the same respective line as said B3872, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 83821, preferably the coding region thereof, conferred the production of or the increase in alanine compared with the wild type control.

The nucleic acid sequence of B3872 from Escherichia coli, e.g. as shown in the respective line in column 5 of Table I, application no. 3, has been published in B. And the activity of the gene product thereof is the activity of transcriptional regulator.

Accordingly, in one embodiment, the process of the present invention for producing aspartate in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3872, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3872, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3872, or a functional equivalent or a homolog thereof as depicted in column 8 of Table II, application no. 3, preferably a homolog or functional equivalent as depicted in column 8 of Table II B, application no. 3, and being depicted in the same respective line as said B3872, and preferably the activity is increased plastidic.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product plastidic with the activity of a “transcriptional regulator”, preferably being encoded by a gene comprising the nucleic acid sequence SEQ ID NO. 83821, preferably the coding region thereof, conferred the production of or the increase in aspartate compared with the wild type control.

The nucleic acid sequence of B3872 from Escherichia coli, e.g. as shown in the respective line in column 5 of Table I, application no. 3, has been published in B. And the activity of the gene product thereof is the activity of transcriptional regulator.

Accordingly, in one embodiment, the process of the present invention for producing serine in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3872, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3872, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3872, or a functional equivalent or a homolog thereof as depicted in column 8 of Table II, application no. 3, preferably a homolog or functional equivalent as depicted in column 8 of Table II B, application no. 3, and being depicted in the same respective line as said B3872, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 83821, preferably the coding region thereof, conferred the production of or the increase in serine compared with the wild type control.

The nucleic acid sequence of B3872 from Escherichia coli, e.g. as shown in the respective line in column 5 of Table I, application no. 3, has been published in B. And the activity of the gene product thereof is the activity of transcriptional regulator.

Accordingly, in one embodiment, the process of the present invention for producing tyrosine in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3872, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3872, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3872, or a functional equivalent or a homolog thereof as depicted in column 8 of Table II, application no. 3, preferably a homolog or functional equivalent as depicted in column 8 of Table II B, application no. 3, and being depicted in the same respective line as said B3872, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 83821, preferably the coding region thereof, conferred the production of or the increase in tyrosine compared with the wild type control.

The nucleic acid sequence of B3989 from Escherichia coli, e.g. as shown in the respective line in column 5 of Table I, application no. 3, has been published in B. And the activity of the gene product thereof is the activity of b3989-protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “b3989-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3989, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3989, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3989, or a functional equivalent or a homolog thereof as depicted in column 8 of Table II, application no. 3, preferably a homolog or functional equivalent as depicted in column 8 of Table II B, application no. 3, and being depicted in the same respective line as said B3989, and preferably the activity is increased non-targeted.

Accordingly, in one embodiment, the molecule which activity is to be increased in the process of the invention is the gene product with an activity as a “b3989-protein”, preferably it is the molecule of section (a) or (b) of this paragraph.

In particular, it was observed that in plants, especially in Arabidopsis thaliana, increasing or generating the activity of a gene product non-targeted with the activity of a “b3989-protein”, preferably being encoded by a gene comprising the nucleic acid sequence SEQ ID NO. 44372, preferably the coding region thereof, conferred the production of or the increase in phenylalanine compared with the wild type control.

The nucleic acid sequence of B3989 from Escherichia coli, e.g. as shown in the respective line in column 5 of Table I, application no. 3, has been published in B. And the activity of the gene product thereof is the activity of b3989-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “b3989-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B3989, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B3989, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B3989, or a functional equivalent or a homolog thereof as depicted in column 8 of Table II, application no. 3, preferably a homolog or functional equivalent as depicted in column 8 of Table II B, application no. 3, and being depicted in the same respective line as said B3989, and preferably the activity is increased non-targeted.

Accordingly, in one embodiment, the molecule which activity is to be increased in the process of the invention is the gene product with an activity as a “b3989-protein”, preferably it is the molecule of section (a) or (b) of this paragraph.

In particular, it was observed that in plants, especially in Arabidopsis thaliana, increasing or generating the activity of a gene product non-targeted with the activity of a “b3989-protein”, preferably being encoded by a gene comprising the nucleic acid sequence SEQ ID NO. 44372, preferably the coding region thereof, conferred the production of or the increase in tyrosine compared with the wild type control.

The nucleic acid sequence of B4012 from Escherichia coli, e.g. as shown in the respective line in column 5 of Table I, application no. 3, has been published in B. And the activity of the gene product thereof is the activity of acetyltransferase.

Accordingly, in one embodiment, the process of the present invention for producing aspartate in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B4012, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B4012, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B4012, or a functional equivalent or a homolog thereof as depicted in column 8 of Table II, application no. 3, preferably a homolog or functional equivalent as depicted in column 8 of Table II B, application no. 3, and being depicted in the same respective line as said B4012, and preferably the activity is increased non-targeted.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 44378, preferably the coding region thereof, conferred the production of or the increase in aspartate compared with the wild type control.

The nucleic acid sequence of B4012 from Escherichia coli, e.g. as shown in the respective line in column 5 of Table I, application no. 3, has been published in B. And the activity of the gene product thereof is the activity of acetyltransferase.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B4012, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B4012, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B4012, or a functional equivalent or a homolog thereof as depicted in column 8 of Table II, application no. 3, preferably a homolog or functional equivalent as depicted in column 8 of Table II B, application no. 3, and being depicted in the same respective line as said B4012, and preferably the activity is increased non-targeted.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 44378, preferably the coding region thereof, conferred the production of or the increase in 5-oxoproline compared with the wild type control.

The nucleic acid sequence of B4024 from Escherichia coli, e.g. as shown in the respective line in column 5 of Table I, application no. 3, has been published in B. And the activity of the gene product thereof is the activity of aspartate kinase.

Accordingly, in one embodiment, the process of the present invention for producing glycine in a non-human organism, like a microorganism or a plant or a part thereof, comprises increasing or generating the activity of a gene product with the activity of a gene product conferring the activity of “aspartate kinase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B4024, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B4024, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B4024, or a functional equivalent or a homolog thereof as depicted in column 8 of Table II, application no. 3, preferably a homolog or functional equivalent as depicted in column 8 of Table II B, application no. 3, and being depicted in the same respective line as said B4024, and preferably the activity is increased plastidic.

Accordingly, in one embodiment, the molecule which activity is to be increased in the process of the invention is the gene product with an activity as a “aspartate kinase”, preferably it is the molecule of section (a) or (b) of this paragraph.

In particular, it was observed that in plants, especially in Arabidopsis thaliana, increasing or generating the activity of a gene product plastidic with the activity of a “aspartate kinase”, preferably being encoded by a gene comprising the nucleic acid sequence SEQ ID NO. 83835, preferably the coding region thereof, conferred the production of or the increase in glycine compared with the wild type control.

The nucleic acid sequence of B4024 from Escherichia coli, e.g. as shown in the respective line in column 5 of Table I, application no. 3, has been published in B. And the activity of the gene product thereof is the activity of aspartate kinase.

Accordingly, in one embodiment, the process of the present invention for producing serine in a non-human organism, like a microorganism or a plant or a part thereof, comprises increasing or generating the activity of a gene product with the activity of a gene product conferring the activity of “aspartate kinase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B4024, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B4024, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B4024, or a functional equivalent or a homolog thereof as depicted in column 8 of Table II, application no. 3, preferably a homolog or functional equivalent as depicted in column 8 of Table II B, application no. 3, and being depicted in the same respective line as said B4024, and preferably the activity is increased plastidic.

Accordingly, in one embodiment, the molecule which activity is to be increased in the process of the invention is the gene product with an activity as a “aspartate kinase”, preferably it is the molecule of section (a) or (b) of this paragraph.

In particular, it was observed that in plants, especially in Arabidopsis thaliana, increasing or generating the activity of a gene product plastidic with the activity of a “aspartate kinase”, preferably being encoded by a gene comprising the nucleic acid sequence SEQ ID NO. 83835, preferably the coding region thereof, conferred the production of or the increase in serine compared with the wild type control.

The nucleic acid sequence of B4029 from Escherichia coli, e.g. as shown in the respective line in column 5 of Table I, application no. 3, has been published in B. And the activity of the gene product thereof is the activity of b4029-protein.

Accordingly, in one embodiment, the process of the present invention for producing alanine in a non-human organism, like a microorganism or a plant or a part thereof, comprises increasing or generating the activity of a gene product with the activity of a gene product conferring the activity of “b4029-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B4029, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B4029, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B4029, or a functional equivalent or a homolog thereof as depicted in column 8 of Table II, application no. 3, preferably a homolog or functional equivalent as depicted in column 8 of Table II B, application no. 3, and being depicted in the same respective line as said B4029, and preferably the activity is increased non-targeted.

Accordingly, in one embodiment, the molecule which activity is to be increased in the process of the invention is the gene product with an activity as a “b4029-protein”, preferably it is the molecule of section (a) or (b) of this paragraph.

In particular, it was observed that in plants, especially in Arabidopsis thaliana, increasing or generating the activity of a gene product non-targeted with the activity of a “b4029-protein”, preferably being encoded by a gene comprising the nucleic acid sequence SEQ ID NO. 10740, preferably the coding region thereof, conferred the production of or the increase in alanine compared with the wild type control.

The nucleic acid sequence of B4029 from Escherichia coli, e.g. as shown in the respective line in column 5 of Table I, application no. 3, has been published in B. And the activity of the gene product thereof is the activity of b4029-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “b4029-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B4029, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B4029, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B4029, or a functional equivalent or a homolog thereof as depicted in column 8 of Table II, application no. 3, preferably a homolog or functional equivalent as depicted in column 8 of Table II B, application no. 3, and being depicted in the same respective line as said B4029, and preferably the activity is increased non-targeted.

Accordingly, in one embodiment, the molecule which activity is to be increased in the process of the invention is the gene product with an activity as a “b4029-protein”, preferably it is the molecule of section (a) or (b) of this paragraph.

In particular, it was observed that in plants, especially in Arabidopsis thaliana, increasing or generating the activity of a gene product non-targeted with the activity of a “b4029-protein”, preferably being encoded by a gene comprising the nucleic acid sequence SEQ ID NO. 10740, preferably the coding region thereof, conferred the production of or the increase in aspartate compared with the wild type control.

The nucleic acid sequence of B4029 from Escherichia coli, e.g. as shown in the respective line in column 5 of Table I, application no. 3, has been published in B. And the activity of the gene product thereof is the activity of b4029-protein.

Accordingly, in one embodiment, the process of the present invention for producing glycine in a non-human organism, like a microorganism or a plant or a part thereof, comprises increasing or generating the activity of a gene product with the activity of a gene product conferring the activity of “b4029-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B4029, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B4029, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B4029, or a functional equivalent or a homolog thereof as depicted in column 8 of Table II, application no. 3, preferably a homolog or functional equivalent as depicted in column 8 of Table II B, application no. 3, and being depicted in the same respective line as said B4029, and preferably the activity is increased non-targeted.

Accordingly, in one embodiment, the molecule which activity is to be increased in the process of the invention is the gene product with an activity as a “b4029-protein”, preferably it is the molecule of section (a) or (b) of this paragraph.

In particular, it was observed that in plants, especially in Arabidopsis thaliana, increasing or generating the activity of a gene product non-targeted with the activity of a “b4029-protein”, preferably being encoded by a gene comprising the nucleic acid sequence SEQ ID NO. 10740, preferably the coding region thereof, conferred the production of or the increase in glycine compared with the wild type control.

The nucleic acid sequence of B4029 from Escherichia coli, e.g. as shown in the respective line in column 5 of Table I, application no. 3, has been published in B. And the activity of the gene product thereof is the activity of b4029-protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “b4029-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B4029, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B4029, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B4029, or a functional equivalent or a homolog thereof as depicted in column 8 of Table II, application no. 3, preferably a homolog or functional equivalent as depicted in column 8 of Table II B, application no. 3, and being depicted in the same respective line as said B4029, and preferably the activity is increased non-targeted.

Accordingly, in one embodiment, the molecule which activity is to be increased in the process of the invention is the gene product with an activity as a “b4029-protein”, preferably it is the molecule of section (a) or (b) of this paragraph.

In particular, it was observed that in plants, especially in Arabidopsis thaliana, increasing or generating the activity of a gene product non-targeted with the activity of a “b4029-protein”, preferably being encoded by a gene comprising the nucleic acid sequence SEQ ID NO. 10740, preferably the coding region thereof, conferred the production of or the increase in phenylalanine compared with the wild type control.

The nucleic acid sequence of B4029 from Escherichia coli, e.g. as shown in the respective line in column 5 of Table I, application no. 3, has been published in B. And the activity of the gene product thereof is the activity of b4029-protein.

Accordingly, in one embodiment, the process of the present invention for producing serine in a non-human organism, like a microorganism or a plant or a part thereof, comprises increasing or generating the activity of a gene product with the activity of a gene product conferring the activity of “b4029-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B4029, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B4029, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B4029, or a functional equivalent or a homolog thereof as depicted in column 8 of Table II, application no. 3, preferably a homolog or functional equivalent as depicted in column 8 of Table II B, application no. 3, and being depicted in the same respective line as said B4029, and preferably the activity is increased non-targeted.

Accordingly, in one embodiment, the molecule which activity is to be increased in the process of the invention is the gene product with an activity as a “b4029-protein”, preferably it is the molecule of section (a) or (b) of this paragraph.

In particular, it was observed that in plants, especially in Arabidopsis thaliana, increasing or generating the activity of a gene product non-targeted with the activity of a “b4029-protein”, preferably being encoded by a gene comprising the nucleic acid sequence SEQ ID NO. 10740, preferably the coding region thereof, conferred the production of or the increase in serine compared with the wild type control.

The nucleic acid sequence of B4029 from Escherichia coli, e.g. as shown in the respective line in column 5 of Table I, application no. 3, has been published in B. And the activity of the gene product thereof is the activity of b4029-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “b4029-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B4029, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B4029, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B4029, or a functional equivalent or a homolog thereof as depicted in column 8 of Table II, application no. 3, preferably a homolog or functional equivalent as depicted in column 8 of Table II B, application no. 3, and being depicted in the same respective line as said B4029, and preferably the activity is increased non-targeted.

Accordingly, in one embodiment, the molecule which activity is to be increased in the process of the invention is the gene product with an activity as a “b4029-protein”, preferably it is the molecule of section (a) or (b) of this paragraph.

In particular, it was observed that in plants, especially in Arabidopsis thaliana, increasing or generating the activity of a gene product non-targeted with the activity of a “b4029-protein”, preferably being encoded by a gene comprising the nucleic acid sequence SEQ ID NO. 10740, preferably the coding region thereof, conferred the production of or the increase in tyrosine compared with the wild type control.

The nucleic acid sequence of B4029 from Escherichia coli, e.g. as shown in the respective line in column 5 of Table I, application no. 3, has been published in B. And the activity of the gene product thereof is the activity of b4029-protein.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “b4029-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B4029, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B4029, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B4029, or a functional equivalent or a homolog thereof as depicted in column 8 of Table II, application no. 3, preferably a homolog or functional equivalent as depicted in column 8 of Table II B, application no. 3, and being depicted in the same respective line as said B4029, and preferably the activity is increased non-targeted.

Accordingly, in one embodiment, the molecule which activity is to be increased in the process of the invention is the gene product with an activity as a “b4029-protein”, preferably it is the molecule of section (a) or (b) of this paragraph.

In particular, it was observed that in plants, especially in Arabidopsis thaliana, increasing or generating the activity of a gene product non-targeted with the activity of a “b4029-protein”, preferably being encoded by a gene comprising the nucleic acid sequence SEQ ID NO. 10740, preferably the coding region thereof, conferred the production of or the increase in 5-oxoproline compared with the wild type control.

The nucleic acid sequence of B4121 from Escherichia coli, e.g. as shown in the respective line in column 5 of Table I, application no. 3, has been published in B. And the activity of the gene product thereof is the activity of b4121-protein.

Accordingly, in one embodiment, the process of the present invention for producing serine in a non-human organism, like a microorganism or a plant or a part thereof, comprises increasing or generating the activity of a gene product with the activity of a gene product conferring the activity of “b4121-protein”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B4121, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B4121, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B4121, or a functional equivalent or a homolog thereof as depicted in column 8 of Table II, application no. 3, preferably a homolog or functional equivalent as depicted in column 8 of Table II B, application no. 3, and being depicted in the same respective line as said B4121, and preferably the activity is increased non-targeted.

Accordingly, in one embodiment, the molecule which activity is to be increased in the process of the invention is the gene product with an activity as a “b4121-protein”, preferably it is the molecule of section (a) or (b) of this paragraph.

In particular, it was observed that in plants, especially in Arabidopsis thaliana, increasing or generating the activity of a gene product non-targeted with the activity of a “b4121-protein”, preferably being encoded by a gene comprising the nucleic acid sequence SEQ ID NO. 44609, preferably the coding region thereof, conferred the production of or the increase in serine compared with the wild type control.

The nucleic acid sequence of B4256 from Escherichia coli, e.g. as shown in the respective line in column 5 of Table I, application no. 3, has been published in B. And the activity of the gene product thereof is the activity of acetyltransferase.

Accordingly, in one embodiment, the process of the present invention for producing aspartate in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B4256, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B4256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B4256, or a functional equivalent or a homolog thereof as depicted in column 8 of Table II, application no. 3, preferably a homolog or functional equivalent as depicted in column 8 of Table II B, application no. 3, and being depicted in the same respective line as said B4256, and preferably the activity is increased non-targeted.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 45321, preferably the coding region thereof, conferred the production of or the increase in aspartate compared with the wild type control.

The nucleic acid sequence of B4256 from Escherichia coli, e.g. as shown in the respective line in column 5 of Table I, application no. 3, has been published in B. And the activity of the gene product thereof is the activity of acetyltransferase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B4256, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B4256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B4256, or a functional equivalent or a homolog thereof as depicted in column 8 of Table II, application no. 3, preferably a homolog or functional equivalent as depicted in column 8 of Table II B, application no. 3, and being depicted in the same respective line as said B4256, and preferably the activity is increased non-targeted.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 45321, preferably the coding region thereof, conferred the production of or the increase in phenylalanine compared with the wild type control.

The nucleic acid sequence of B4256 from Escherichia coli, e.g. as shown in the respective line in column 5 of Table I, application no. 3, has been published in B. And the activity of the gene product thereof is the activity of acetyltransferase.

Accordingly, in one embodiment, the process of the present invention for producing serine in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B4256, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B4256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B4256, or a functional equivalent or a homolog thereof as depicted in column 8 of Table II, application no. 3, preferably a homolog or functional equivalent as depicted in column 8 of Table II B, application no. 3, and being depicted in the same respective line as said B4256, and preferably the activity is increased non-targeted.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 45321, preferably the coding region thereof, conferred the production of or the increase in serine compared with the wild type control.

The nucleic acid sequence of B4256 from Escherichia coli, e.g. as shown in the respective line in column 5 of Table I, application no. 3, has been published in B. And the activity of the gene product thereof is the activity of acetyltransferase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said B4256, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said B4256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said B4256, or a functional equivalent or a homolog thereof as depicted in column 8 of Table II, application no. 3, preferably a homolog or functional equivalent as depicted in column 8 of Table II B, application no. 3, and being depicted in the same respective line as said B4256, and preferably the activity is increased non-targeted.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 45321, preferably the coding region thereof, conferred the production of or the increase in tyrosine compared with the wild type control.

The nucleic acid sequence of C_pp004096192r from Physcomitrella patens, e.g. as shown in the respective line in column 5 of Table I, application no. 3, is unpublished. And the activity of the gene product thereof is the activity of thioredoxin.

Accordingly, in one embodiment, the process of the present invention for producing serine in a non-human organism, like a microorganism or a plant or a part thereof, comprises increasing or generating the activity of a gene product with the activity of a gene product conferring the activity of “thioredoxin”, especially from Physcomitrella patens or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said C_pp004096192r, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said C_pp004096192r, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said C_pp004096192r, or a functional equivalent or a homolog thereof as depicted in column 8 of Table II, application no. 3, preferably a homolog or functional equivalent as depicted in column 8 of Table II B, application no. 3, and being depicted in the same respective line as said C_pp004096192r, and preferably the activity is increased non-targeted.

Accordingly, in one embodiment, the molecule which activity is to be increased in the process of the invention is the gene product with an activity as a “thioredoxin”, preferably it is the molecule of section (a) or (b) of this paragraph.

In particular, it was observed that in plants, especially in Arabidopsis thaliana, increasing or generating the activity of a gene product non-targeted with the activity of a “thioredoxin”, preferably being encoded by a gene comprising the nucleic acid sequence SEQ ID NO. 45757, preferably the coding region thereof, conferred the production of or the increase in serine compared with the wild type control.

The nucleic acid sequence of D90900 from Synechocystis sp., e.g. as shown in the respective line in column 5 of Table I, application no. 3, has been published in C. And the activity of the gene product thereof is the activity of lysine decarboxylase.

Accordingly, in one embodiment, the process of the present invention for producing alanine in a non-human organism, like a microorganism or a plant or a part thereof, comprises increasing or generating the activity of a gene product with the activity of a gene product conferring the activity of “lysine decarboxylase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said D90900, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said D90900, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said D90900, or a functional equivalent or a homolog thereof as depicted in column 8 of Table II, application no. 3, preferably a homolog or functional equivalent as depicted in column 8 of Table II B, application no. 3, and being depicted in the same respective line as said D90900, and preferably the activity is increased plastidic.

Accordingly, in one embodiment, the molecule which activity is to be increased in the process of the invention is the gene product with an activity as a “lysine decarboxylase”, preferably it is the molecule of section (a) or (b) of this paragraph.

In particular, it was observed that in plants, especially in Arabidopsis thaliana, increasing or generating the activity of a gene product plastidic with the activity of a “lysine decarboxylase”, preferably being encoded by a gene comprising the nucleic acid sequence SEQ ID NO. 84079, preferably the coding region thereof, conferred the production of or the increase in alanine compared with the wild type control.

The nucleic acid sequence of GM02LC11114 from Glycine max, e.g. as shown in the respective line in column 5 of Table I, application no. 3, is unpublished. And the activity of the gene product thereof is the activity of GM02LC11114-protein.

Accordingly, in one embodiment, the process of the present invention for producing asparagine in a non-human organism, like a microorganism or a plant or a part thereof, comprises increasing or generating the activity of a gene product with the activity of a gene product conferring the activity of “GM02LC11114-protein”, especially from Glycine max or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said GM02LC11114, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said GM02LC11114, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said GM02LC11114, or a functional equivalent or a homolog thereof as depicted in column 8 of Table II, application no. 3, preferably a homolog or functional equivalent as depicted in column 8 of Table II B, application no. 3, and being depicted in the same respective line as said GM02LC11114, and preferably the activity is increased non-targeted.

Accordingly, in one embodiment, the molecule which activity is to be increased in the process of the invention is the gene product with an activity as a “GM02LC11114-protein”, preferably it is the molecule of section (a) or (b) of this paragraph.

In particular, it was observed that in plants, especially in Arabidopsis thaliana, increasing or generating the activity of a gene product non-targeted with the activity of a “GM02LC11114-protein”, preferably being encoded by a gene comprising the nucleic acid sequence SEQ ID NO. 45795, preferably the coding region thereof, conferred the production of or the increase in asparagine compared with the wild type control.

The nucleic acid sequence of GM02LC11114 from Glycine max, e.g. as shown in the respective line in column 5 of Table I, application no. 3, is unpublished. And the activity of the gene product thereof is the activity of GM02LC11114-protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “GM02LC11114-protein”, especially from Glycine max or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said GM02LC11114, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said GM02LC11114, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said GM02LC11114, or a functional equivalent or a homolog thereof as depicted in column 8 of Table II, application no. 3, preferably a homolog or functional equivalent as depicted in column 8 of Table II B, application no. 3, and being depicted in the same respective line as said GM02LC11114, and preferably the activity is increased non-targeted.

Accordingly, in one embodiment, the molecule which activity is to be increased in the process of the invention is the gene product with an activity as a “GM02LC11114-protein”, preferably it is the molecule of section (a) or (b) of this paragraph.

In particular, it was observed that in plants, especially in Arabidopsis thaliana, increasing or generating the activity of a gene product non-targeted with the activity of a “GM02LC11114-protein”, preferably being encoded by a gene comprising the nucleic acid sequence SEQ ID NO. 45795, preferably the coding region thereof, conferred the production of or the increase in phenylalanine compared with the wild type control.

The nucleic acid sequence of GM02LC12622 from Glycine max, e.g. as shown in the respective line in column 5 of Table I, application no. 3, is unpublished. And the activity of the gene product thereof is the activity of glutaredoxin.

Accordingly, in one embodiment, the process of the present invention for producing alanine in a non-human organism, like a microorganism or a plant or a part thereof, comprises increasing or generating the activity of a gene product with the activity of a gene product conferring the activity of “glutaredoxin”, especially from Glycine max or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said GM02LC12622, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said GM02LC12622, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said GM02LC12622, or a functional equivalent or a homolog thereof as depicted in column 8 of Table II, application no. 3, preferably a homolog or functional equivalent as depicted in column 8 of Table II B, application no. 3, and being depicted in the same respective line as said GM02LC12622, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 10811, preferably the coding region thereof, conferred the production of or the increase in alanine compared with the wild type control.

The nucleic acid sequence of GM02LC12622 from Glycine max, e.g. as shown in the respective line in column 5 of Table I, application no. 3, is unpublished. And the activity of the gene product thereof is the activity of glutaredoxin.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “glutaredoxin”, especially from Glycine max or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said GM02LC12622, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said GM02LC12622, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said GM02LC12622, or a functional equivalent or a homolog thereof as depicted in column 8 of Table II, application no. 3, preferably a homolog or functional equivalent as depicted in column 8 of Table II B, application no. 3, and being depicted in the same respective line as said GM02LC12622, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 10811, preferably the coding region thereof, conferred the production of or the increase in phenylalanine compared with the wild type control.

The nucleic acid sequence of GM02LC12622 from Glycine max, e.g. as shown in the respective line in column 5 of Table I, application no. 3, is unpublished. And the activity of the gene product thereof is the activity of glutaredoxin.

Accordingly, in one embodiment, the process of the present invention for producing tyrosine in a non-human organism, like a microorganism or a plant or a part thereof, comprises increasing or generating the activity of a gene product with the activity of a gene product conferring the activity of “glutaredoxin”, especially from Glycine max or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said GM02LC12622, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said GM02LC12622, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said GM02LC12622, or a functional equivalent or a homolog thereof as depicted in column 8 of Table II, application no. 3, preferably a homolog or functional equivalent as depicted in column 8 of Table II B, application no. 3, and being depicted in the same respective line as said GM02LC12622, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 10811, preferably the coding region thereof, conferred the production of or the increase in tyrosine compared with the wild type control.

The nucleic acid sequence of GM02LC15313 from Glycine max, e.g. as shown in the respective line in column 5 of Table I, application no. 3, is unpublished. And the activity of the gene product thereof is the activity of histone H2A.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “histone H2A”, especially from Glycine max or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said GM02LC15313, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said GM02LC15313, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said GM02LC15313, or a functional equivalent or a homolog thereof as depicted in column 8 of Table II, application no. 3, preferably a homolog or functional equivalent as depicted in column 8 of Table II B, application no. 3, and being depicted in the same respective line as said GM02LC15313, and preferably the activity is increased non-targeted.

Accordingly, in one embodiment, the molecule which activity is to be increased in the process of the invention is the gene product with an activity as a “histone H2A”, preferably it is the molecule of section (a) or (b) of this paragraph.

In particular, it was observed that in plants, especially in Arabidopsis thaliana, increasing or generating the activity of a gene product non-targeted with the activity of a “histone H2A”, preferably being encoded by a gene comprising the nucleic acid sequence SEQ ID NO. 45897, preferably the coding region thereof, conferred the production of or the increase in phenylalanine compared with the wild type control.

The nucleic acid sequence of GM02LC15313 from Glycine max, e.g. as shown in the respective line in column 5 of Table I, application no. 3, is unpublished. And the activity of the gene product thereof is the activity of histone H2A.

Accordingly, in one embodiment, the process of the present invention for producing tyrosine in a non-human organism, like a microorganism or a plant or a part thereof, comprises increasing or generating the activity of a gene product with the activity of a gene product conferring the activity of “histone H2A”, especially from Glycine max or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said GM02LC15313, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said GM02LC15313, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said GM02LC15313, or a functional equivalent or a homolog thereof as depicted in column 8 of Table II, application no. 3, preferably a homolog or functional equivalent as depicted in column 8 of Table II B, application no. 3, and being depicted in the same respective line as said GM02LC15313, and preferably the activity is increased non-targeted.

Accordingly, in one embodiment, the molecule which activity is to be increased in the process of the invention is the gene product with an activity as a “histone H2A”, preferably it is the molecule of section (a) or (b) of this paragraph.

In particular, it was observed that in plants, especially in Arabidopsis thaliana, increasing or generating the activity of a gene product non-targeted with the activity of a “histone H2A”, preferably being encoded by a gene comprising the nucleic acid sequence SEQ ID NO. 45897, preferably the coding region thereof, conferred the production of or the increase in tyrosine compared with the wild type control.

The nucleic acid sequence of GM02LC17485 from Glycine max, e.g. as shown in the respective line in column 5 of Table I, application no. 3, is unpublished. And the activity of the gene product thereof is the activity of GM02LC17485-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “GM02LC17485-protein”, especially from Glycine max or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said GM02LC17485, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said GM02LC17485, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said GM02LC17485, or a functional equivalent or a homolog thereof as depicted in column 8 of Table II, application no. 3, preferably a homolog or functional equivalent as depicted in column 8 of Table II B, application no. 3, and being depicted in the same respective line as said GM02LC17485, and preferably the activity is increased non-targeted.

Accordingly, in one embodiment, the molecule which activity is to be increased in the process of the invention is the gene product with an activity as a “GM02LC17485-protein”, preferably it is the molecule of section (a) or (b) of this paragraph.

In particular, it was observed that in plants, especially in Arabidopsis thaliana, increasing or generating the activity of a gene product non-targeted with the activity of a “GM02LC17485-protein”, preferably being encoded by a gene comprising the nucleic acid sequence SEQ ID NO. 46405, preferably the coding region thereof, conferred the production of or the increase in aspartate compared with the wild type control.

The nucleic acid sequence of GM02LC17556 from Glycine max, e.g. as shown in the respective line in column 5 of Table I, application no. 3, is unpublished. And the activity of the gene product thereof is the activity of RNA binding protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “RNA binding protein”, especially from Glycine max or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said GM02LC17556, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said GM02LC17556, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said GM02LC17556, or a functional equivalent or a homolog thereof as depicted in column 8 of Table II, application no. 3, preferably a homolog or functional equivalent as depicted in column 8 of Table II B, application no. 3, and being depicted in the same respective line as said GM02LC17556, and preferably the activity is increased non-targeted.

Accordingly, in one embodiment, the molecule which activity is to be increased in the process of the invention is the gene product with an activity as a “RNA binding protein”, preferably it is the molecule of section (a) or (b) of this paragraph.

In particular, it was observed that in plants, especially in Arabidopsis thaliana, increasing or generating the activity of a gene product non-targeted with the activity of a “RNA binding protein”, preferably being encoded by a gene comprising the nucleic acid sequence SEQ ID NO. 46515, preferably the coding region thereof, conferred the production of or the increase in phenylalanine compared with the wild type control.

The nucleic acid sequence of GM02LC17556 from Glycine max, e.g. as shown in the respective line in column 5 of Table I, application no. 3, is unpublished. And the activity of the gene product thereof is the activity of RNA binding protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “RNA binding protein”, especially from Glycine max or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said GM02LC17556, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said GM02LC17556, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said GM02LC17556, or a functional equivalent or a homolog thereof as depicted in column 8 of Table II, application no. 3, preferably a homolog or functional equivalent as depicted in column 8 of Table II B, application no. 3, and being depicted in the same respective line as said GM02LC17556, and preferably the activity is increased non-targeted.

Accordingly, in one embodiment, the molecule which activity is to be increased in the process of the invention is the gene product with an activity as a “RNA binding protein”, preferably it is the molecule of section (a) or (b) of this paragraph.

In particular, it was observed that in plants, especially in Arabidopsis thaliana, increasing or generating the activity of a gene product non-targeted with the activity of a “RNA binding protein”, preferably being encoded by a gene comprising the nucleic acid sequence SEQ ID NO. 46515, preferably the coding region thereof, conferred the production of or the increase in tyrosine compared with the wild type control.

The nucleic acid sequence of GM02LC21368 from Glycine max, e.g. as shown in the respective line in column 5 of Table I, application no. 3, is unpublished. And the activity of the gene product thereof is the activity of transcription factor.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said GM02LC21368, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said GM02LC21368, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said GM02LC21368, or a functional equivalent or a homolog thereof as depicted in column 8 of Table II, application no. 3, preferably a homolog or functional equivalent as depicted in column 8 of Table II B, application no. 3, and being depicted in the same respective line as said GM02LC21368, and preferably the activity is increased non-targeted.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 84198, preferably the coding region thereof, conferred the production of or the increase in phenylalanine compared with the wild type control.

The nucleic acid sequence of GM02LC21368 from Glycine max, e.g. as shown in the respective line in column 5 of Table I, application no. 3, is unpublished. And the activity of the gene product thereof is the activity of transcription factor.

Accordingly, in one embodiment, the process of the present invention for producing serine in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said GM02LC21368, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said GM02LC21368, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said GM02LC21368, or a functional equivalent or a homolog thereof as depicted in column 8 of Table II, application no. 3, preferably a homolog or functional equivalent as depicted in column 8 of Table II B, application no. 3, and being depicted in the same respective line as said GM02LC21368, and preferably the activity is increased non-targeted.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 84198, preferably the coding region thereof, conferred the production of or the increase in serine compared with the wild type control.

The nucleic acid sequence of GM02LC21368 from Glycine max, e.g. as shown in the respective line in column 5 of Table I, application no. 3, is unpublished. And the activity of the gene product thereof is the activity of transcription factor.

Accordingly, in one embodiment, the process of the present invention for producing tyrosine in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said GM02LC21368, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said GM02LC21368, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said GM02LC21368, or a functional equivalent or a homolog thereof as depicted in column 8 of Table II, application no. 3, preferably a homolog or functional equivalent as depicted in column 8 of Table II B, application no. 3, and being depicted in the same respective line as said GM02LC21368, and preferably the activity is increased non-targeted.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 84198, preferably the coding region thereof, conferred the production of or the increase in tyrosine compared with the wild type control.

The nucleic acid sequence of GM02LC44512 from Glycine max, e.g. as shown in the respective line in column 5 of Table I, application no. 3, is unpublished. And the activity of the gene product thereof is the activity of cyclin D.

Accordingly, in one embodiment, the process of the present invention for producing alanine in a non-human organism, like a microorganism or a plant or a part thereof, comprises increasing or generating the activity of a gene product with the activity of a gene product conferring the activity of “cyclin D”, especially from Glycine max or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said GM02LC44512, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said GM02LC44512, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said GM02LC44512, or a functional equivalent or a homolog thereof as depicted in column 8 of Table II, application no. 3, preferably a homolog or functional equivalent as depicted in column 8 of Table II B, application no. 3, and being depicted in the same respective line as said GM02LC44512, and preferably the activity is increased non-targeted.

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 46850, preferably the coding region thereof, conferred the production of or the increase in alanine compared with the wild type control.

The nucleic acid sequence of GM02LC44512 from Glycine max, e.g. as shown in the respective line in column 5 of Table I, application no. 3, is unpublished. And the activity of the gene product thereof is the activity of cyclin D.

Accordingly, in one embodiment, the process of the present invention for producing serine in a non-human organism, like a microorganism or a plant or a part thereof, comprises increasing or generating the activity of a gene product with the activity of a gene product conferring the activity of “cyclin D”, especially from Glycine max or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said GM02LC44512, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said GM02LC44512, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said GM02LC44512, or a functional equivalent or a homolog thereof as depicted in column 8 of Table II, application no. 3, preferably a homolog or functional equivalent as depicted in column 8 of Table II B, application no. 3, and being depicted in the same respective line as said GM02LC44512, and preferably the activity is increased non-targeted.

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 46850, preferably the coding region thereof, conferred the production of or the increase in serine compared with the wild type control.

The nucleic acid sequence of NZ_AAAU02000016.150 from Azotobacter vinelandii, e.g. as shown in the respective line in column 5 of Table I, application no. 3, has been published in D. And the activity of the gene product thereof is the activity of glutaredoxin.

Accordingly, in one embodiment, the process of the present invention for producing alanine in a non-human organism, like a microorganism or a plant or a part thereof, comprises increasing or generating the activity of a gene product with the activity of a gene product conferring the activity of “glutaredoxin”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said NZ_AAAU02000016.150, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said NZ_AAAU02000016.150, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said NZ_AAAU02000016.150, or a functional equivalent or a homolog thereof as depicted in column 8 of Table II, application no. 3, preferably a homolog or functional equivalent as depicted in column 8 of Table II B, application no. 3, and being depicted in the same respective line as said NZ_AAAU02000016.150, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 47159, preferably the coding region thereof, conferred the production of or the increase in alanine compared with the wild type control.

The nucleic acid sequence of NZ_AAAU02000016.150 from Azotobacter vinelandii, e.g. as shown in the respective line in column 5 of Table I, application no. 3, has been published in D. And the activity of the gene product thereof is the activity of glutaredoxin.

Accordingly, in one embodiment, the process of the present invention for producing glycine in a non-human organism, like a microorganism or a plant or a part thereof, comprises increasing or generating the activity of a gene product with the activity of a gene product conferring the activity of “glutaredoxin”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said NZ_AAAU02000016.150, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said NZ_AAAU02000016.150, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said NZ_AAAU02000016.150, or a functional equivalent or a homolog thereof as depicted in column 8 of Table II, application no. 3, preferably a homolog or functional equivalent as depicted in column 8 of Table II B, application no. 3, and being depicted in the same respective line as said NZ_AAAU02000016.150, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 47159, preferably the coding region thereof, conferred the production of or the increase in glycine compared with the wild type control.

The nucleic acid sequence of NZ_AAAU02000016.150 from Azotobacter vinelandii, e.g. as shown in the respective line in column 5 of Table I, application no. 3, has been published in D. And the activity of the gene product thereof is the activity of glutaredoxin.

Accordingly, in one embodiment, the process of the present invention for producing serine in a non-human organism, like a microorganism or a plant or a part thereof, comprises increasing or generating the activity of a gene product with the activity of a gene product conferring the activity of “glutaredoxin”, especially from Azotobacter vinelandii or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said NZ_AAAU02000016.150, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said NZ_AAAU02000016.150, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said NZ_AAAU02000016.150, or a functional equivalent or a homolog thereof as depicted in column 8 of Table II, application no. 3, preferably a homolog or functional equivalent as depicted in column 8 of Table II B, application no. 3, and being depicted in the same respective line as said NZ_AAAU02000016.150, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 47159, preferably the coding region thereof, conferred the production of or the increase in serine compared with the wild type control.

The nucleic acid sequence of S_pp015018333r from Physcomitrella patens, e.g. as shown in the respective line in column 5 of Table I, application no. 3, is unpublished. And the activity of the gene product thereof is the activity of s_pp015018333r-protein.

Accordingly, in one embodiment, the process of the present invention for producing asparagine in a non-human organism, like a microorganism or a plant or a part thereof, comprises increasing or generating the activity of a gene product with the activity of a gene product conferring the activity of “s_pp015018333r-protein”, especially from Physcomitrella patens or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said S_pp015018333r, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said S_pp015018333r, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said S_pp015018333r, or a functional equivalent or a homolog thereof as depicted in column 8 of Table II, application no. 3, preferably a homolog or functional equivalent as depicted in column 8 of Table II B, application no. 3, and being depicted in the same respective line as said S_pp015018333r, and preferably the activity is increased non-targeted.

Accordingly, in one embodiment, the molecule which activity is to be increased in the process of the invention is the gene product with an activity as a “s_pp015018333r-protein”, preferably it is the molecule of section (a) or (b) of this paragraph.

In particular, it was observed that in plants, especially in Arabidopsis thaliana, increasing or generating the activity of a gene product non-targeted with the activity of a “s_pp015018333r-protein”, preferably being encoded by a gene comprising the nucleic acid sequence SEQ ID NO. 59851, preferably the coding region thereof, conferred the production of or the increase in asparagine compared with the wild type control.

The nucleic acid sequence of S_pp015018333r from Physcomitrella patens, e.g. as shown in the respective line in column 5 of Table I, application no. 3, is unpublished. And the activity of the gene product thereof is the activity of s_pp015018333r-protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “s_pp015018333r-protein”, especially from Physcomitrella patens or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said S_pp015018333r, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said S_pp015018333r, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said S_pp015018333r, or a functional equivalent or a homolog thereof as depicted in column 8 of Table II, application no. 3, preferably a homolog or functional equivalent as depicted in column 8 of Table II B, application no. 3, and being depicted in the same respective line as said S_pp015018333r, and preferably the activity is increased non-targeted.

Accordingly, in one embodiment, the molecule which activity is to be increased in the process of the invention is the gene product with an activity as a “s_pp015018333r-protein”, preferably it is the molecule of section (a) or (b) of this paragraph.

In particular, it was observed that in plants, especially in Arabidopsis thaliana, increasing or generating the activity of a gene product non-targeted with the activity of a “s_pp015018333r-protein”, preferably being encoded by a gene comprising the nucleic acid sequence SEQ ID NO. 59851, preferably the coding region thereof, conferred the production of or the increase in phenylalanine compared with the wild type control.

The nucleic acid sequence of S_pp015018333r from Physcomitrella patens, e.g. as shown in the respective line in column 5 of Table I, application no. 3, is unpublished. And the activity of the gene product thereof is the activity of s_pp015018333r-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “s_pp015018333r-protein”, especially from Physcomitrella patens or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said S_pp015018333r, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said S_pp015018333r, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said S_pp015018333r, or a functional equivalent or a homolog thereof as depicted in column 8 of Table II, application no. 3, preferably a homolog or functional equivalent as depicted in column 8 of Table II B, application no. 3, and being depicted in the same respective line as said S_pp015018333r, and preferably the activity is increased non-targeted.

Accordingly, in one embodiment, the molecule which activity is to be increased in the process of the invention is the gene product with an activity as a “s_pp015018333r-protein”, preferably it is the molecule of section (a) or (b) of this paragraph.

In particular, it was observed that in plants, especially in Arabidopsis thaliana, increasing or generating the activity of a gene product non-targeted with the activity of a “s_pp015018333r-protein”, preferably being encoded by a gene comprising the nucleic acid sequence SEQ ID NO. 59851, preferably the coding region thereof, conferred the production of or the increase in tyrosine compared with the wild type control.

The nucleic acid sequence of S_pp015018333r from Physcomitrella patens, e.g. as shown in the respective line in column 5 of Table I, application no. 3, is unpublished. And the activity of the gene product thereof is the activity of s_pp015018333r-protein.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “s_pp015018333r-protein”, especially from Physcomitrella patens or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said S_pp015018333r, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said S_pp015018333r, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said S_pp015018333r, or a functional equivalent or a homolog thereof as depicted in column 8 of Table II, application no. 3, preferably a homolog or functional equivalent as depicted in column 8 of Table II B, application no. 3, and being depicted in the same respective line as said S_pp015018333r, and preferably the activity is increased non-targeted.

Accordingly, in one embodiment, the molecule which activity is to be increased in the process of the invention is the gene product with an activity as a “s_pp015018333r-protein”, preferably it is the molecule of section (a) or (b) of this paragraph.

In particular, it was observed that in plants, especially in Arabidopsis thaliana, increasing or generating the activity of a gene product non-targeted with the activity of a “s_pp015018333r-protein”, preferably being encoded by a gene comprising the nucleic acid sequence SEQ ID NO. 59851, preferably the coding region thereof, conferred the production of or the increase in 5-oxoproline compared with the wild type control.

The nucleic acid sequence of Sll0080 from Synechocystis sp., e.g. as shown in the respective line in column 5 of Table I, application no. 3, has been published in C. And the activity of the gene product thereof is the activity of N-acetyl-gamma-glutamyl-phosphate reductase.

Accordingly, in one embodiment, the process of the present invention for producing citrulline in a non-human organism, like a microorganism or a plant or a part thereof, comprises increasing or generating the activity of a gene product with the activity of a gene product conferring the activity of “N-acetyl-gamma-glutamyl-phosphate reductase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll0080, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll0080, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll0080, or a functional equivalent or a homolog thereof as depicted in column 8 of Table II, application no. 3, preferably a homolog or functional equivalent as depicted in column 8 of Table II B, application no. 3, and being depicted in the same respective line as said Sll0080, and preferably the activity is increased plastidic.

Accordingly, in one embodiment, the molecule which activity is to be increased in the process of the invention is the gene product with an activity as a “N-acetyl-gamma-glutamyl-phosphate reductase”, preferably it is the molecule of section (a) or (b) of this paragraph.

In particular, it was observed that in plants, especially in Arabidopsis thaliana, increasing or generating the activity of a gene product plastidic with the activity of a “N-acetyl-gamma-glutamyl-phosphate reductase”, preferably being encoded by a gene comprising the nucleic acid sequence SEQ ID NO. 47566, preferably the coding region thereof, conferred the production of or the increase in citrulline compared with the wild type control.

The nucleic acid sequence of Sll0228 from Synechocystis sp., e.g. as shown in the respective line in column 5 of Table I, application no. 3, has been published in C. And the activity of the gene product thereof is the activity of agmatinase.

Accordingly, in one embodiment, the process of the present invention for producing asparagine in a non-human organism, like a microorganism or a plant or a part thereof, comprises increasing or generating the activity of a gene product with the activity of a gene product conferring the activity of “agmatinase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll0228, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll0228, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll0228, or a functional equivalent or a homolog thereof as depicted in column 8 of Table II, application no. 3, preferably a homolog or functional equivalent as depicted in column 8 of Table II B, application no. 3, and being depicted in the same respective line as said Sll0228, and preferably the activity is increased plastidic.

Accordingly, in one embodiment, the molecule which activity is to be increased in the process of the invention is the gene product with an activity as a “agmatinase”, preferably it is the molecule of section (a) or (b) of this paragraph.

In particular, it was observed that in plants, especially in Arabidopsis thaliana, increasing or generating the activity of a gene product plastidic with the activity of a “agmatinase”, preferably being encoded by a gene comprising the nucleic acid sequence SEQ ID NO. 84265, preferably the coding region thereof, conferred the production of or the increase in asparagine compared with the wild type control.

The nucleic acid sequence of Sll0228 from Synechocystis sp., e.g. as shown in the respective line in column 5 of Table I, application no. 3, has been published in C. And the activity of the gene product thereof is the activity of agmatinase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “agmatinase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll0228, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll0228, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll0228, or a functional equivalent or a homolog thereof as depicted in column 8 of Table II, application no. 3, preferably a homolog or functional equivalent as depicted in column 8 of Table II B, application no. 3, and being depicted in the same respective line as said Sll0228, and preferably the activity is increased plastidic.

Accordingly, in one embodiment, the molecule which activity is to be increased in the process of the invention is the gene product with an activity as a “agmatinase”, preferably it is the molecule of section (a) or (b) of this paragraph.

In particular, it was observed that in plants, especially in Arabidopsis thaliana, increasing or generating the activity of a gene product plastidic with the activity of a “agmatinase”, preferably being encoded by a gene comprising the nucleic acid sequence SEQ ID NO. 84265, preferably the coding region thereof, conferred the production of or the increase in tyrosine compared with the wild type control.

The nucleic acid sequence of Sll0240 from Synechocystis sp., e.g. as shown in the respective line in column 5 of Table I, application no. 3, has been published in C. And the activity of the gene product thereof is the activity of ABC transporter ATP binding component.

Accordingly, in one embodiment, the process of the present invention for producing alanine in a non-human organism, like a microorganism or a plant or a part thereof, comprises increasing or generating the activity of a gene product with the activity of a gene product conferring the activity of “ABC transporter ATP binding component”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll0240, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll0240, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll0240, or a functional equivalent or a homolog thereof as depicted in column 8 of Table II, application no. 3, preferably a homolog or functional equivalent as depicted in column 8 of Table II B, application no. 3, and being depicted in the same respective line as said Sll0240, and preferably the activity is increased plastidic.

Accordingly, in one embodiment, the molecule which activity is to be increased in the process of the invention is the gene product with an activity as a “ABC transporter ATP binding component”, preferably it is the molecule of section (a) or (b) of this paragraph.

In particular, it was observed that in plants, especially in Arabidopsis thaliana, increasing or generating the activity of a gene product plastidic with the activity of a “ABC transporter ATP binding component”, preferably being encoded by a gene comprising the nucleic acid sequence SEQ ID NO. 84441, preferably the coding region thereof, conferred the production of or the increase in alanine compared with the wild type control.

The nucleic acid sequence of Sll0248 from Synechocystis sp., e.g. as shown in the respective line in column 5 of Table I, application no. 3, has been published in C. And the activity of the gene product thereof is the activity of flavodoxin.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “flavodoxin”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll0248, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll0248, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll0248, or a functional equivalent or a homolog thereof as depicted in column 8 of Table II, application no. 3, preferably a homolog or functional equivalent as depicted in column 8 of Table II B, application no. 3, and being depicted in the same respective line as said Sll0248, and preferably the activity is increased plastidic.

Accordingly, in one embodiment, the molecule which activity is to be increased in the process of the invention is the gene product with an activity as a “flavodoxin”, preferably it is the molecule of section (a) or (b) of this paragraph.

In particular, it was observed that in plants, especially in Arabidopsis thaliana, increasing or generating the activity of a gene product plastidic with the activity of a “flavodoxin”, preferably being encoded by a gene comprising the nucleic acid sequence SEQ ID NO. 49143, preferably the coding region thereof, conferred the production of or the increase in phenylalanine compared with the wild type control.

The nucleic acid sequence of Sll0248 from Synechocystis sp., e.g. as shown in the respective line in column 5 of Table I, application no. 3, has been published in C. And the activity of the gene product thereof is the activity of flavodoxin.

Accordingly, in one embodiment, the process of the present invention for producing tyrosine in a non-human organism, like a microorganism or a plant or a part thereof, comprises increasing or generating the activity of a gene product with the activity of a gene product conferring the activity of “flavodoxin”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll0248, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll0248, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll0248, or a functional equivalent or a homolog thereof as depicted in column 8 of Table II, application no. 3, preferably a homolog or functional equivalent as depicted in column 8 of Table II B, application no. 3, and being depicted in the same respective line as said Sll0248, and preferably the activity is increased plastidic.

Accordingly, in one embodiment, the molecule which activity is to be increased in the process of the invention is the gene product with an activity as a “flavodoxin”, preferably it is the molecule of section (a) or (b) of this paragraph.

In particular, it was observed that in plants, especially in Arabidopsis thaliana, increasing or generating the activity of a gene product plastidic with the activity of a “flavodoxin”, preferably being encoded by a gene comprising the nucleic acid sequence SEQ ID NO. 49143, preferably the coding region thereof, conferred the production of or the increase in tyrosine compared with the wild type control.

The nucleic acid sequence of Sll0281 from Synechocystis sp., e.g. as shown in the respective line in column 5 of Table I, application no. 3, has been published in C. And the activity of the gene product thereof is the activity of sll0281-protein.

Accordingly, in one embodiment, the process of the present invention for producing alanine in a non-human organism, like a microorganism or a plant or a part thereof, comprises increasing or generating the activity of a gene product with the activity of a gene product conferring the activity of “sll0281-protein”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll0281, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll0281, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll0281, or a functional equivalent or a homolog thereof as depicted in column 8 of Table II, application no. 3, preferably a homolog or functional equivalent as depicted in column 8 of Table II B, application no. 3, and being depicted in the same respective line as said Sll0281, and preferably the activity is increased plastidic.

Accordingly, in one embodiment, the molecule which activity is to be increased in the process of the invention is the gene product with an activity as a “sll0281-protein”, preferably it is the molecule of section (a) or (b) of this paragraph.

In particular, it was observed that in plants, especially in Arabidopsis thaliana, increasing or generating the activity of a gene product plastidic with the activity of a “sll0281-protein”, preferably being encoded by a gene comprising the nucleic acid sequence SEQ ID NO. 84865, preferably the coding region thereof, conferred the production of or the increase in alanine compared with the wild type control.

The nucleic acid sequence of Sll0378 from Synechocystis sp., e.g. as shown in the respective line in column 5 of Table I, application no. 3, has been published in C. And the activity of the gene product thereof is the activity of uroporphyrin-111C-methyltransferase.

Accordingly, in one embodiment, the process of the present invention for producing serine in a non-human organism, like a microorganism or a plant or a part thereof, comprises increasing or generating the activity of a gene product with the activity of a gene product conferring the activity of “uroporphyrin-111C-methyltransferase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll0378, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll0378, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll0378, or a functional equivalent or a homolog thereof as depicted in column 8 of Table II, application no. 3, preferably a homolog or functional equivalent as depicted in column 8 of Table II B, application no. 3, and being depicted in the same respective line as said Sll0378, and preferably the activity is increased plastidic.

Accordingly, in one embodiment, the molecule which activity is to be increased in the process of the invention is the gene product with an activity as a “uroporphyrin-III C-methyltransferase”, preferably it is the molecule of section (a) or (b) of this paragraph.

In particular, it was observed that in plants, especially in Arabidopsis thaliana, increasing or generating the activity of a gene product plastidic with the activity of a “uroporphyrin-III C-methyltransferase”, preferably being encoded by a gene comprising the nucleic acid sequence SEQ ID NO. 84869, preferably the coding region thereof, conferred the production of or the increase in serine compared with the wild type control.

The nucleic acid sequence of Sll0420 from Synechocystis sp., e.g. as shown in the respective line in column 5 of Table I, application no. 3, has been published in C. And the activity of the gene product thereof is the activity of urease subunit.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “urease subunit”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll0420, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll0420, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll0420, or a functional equivalent or a homolog thereof as depicted in column 8 of Table II, application no. 3, preferably a homolog or functional equivalent as depicted in column 8 of Table II B, application no. 3, and being depicted in the same respective line as said Sll0420, and preferably the activity is increased non-targeted.

Accordingly, in one embodiment, the molecule which activity is to be increased in the process of the invention is the gene product with an activity as a “urease subunit”, preferably it is the molecule of section (a) or (b) of this paragraph.

In particular, it was observed that in plants, especially in Arabidopsis thaliana, increasing or generating the activity of a gene product non-targeted with the activity of a “urease subunit”, preferably being encoded by a gene comprising the nucleic acid sequence SEQ ID NO. 50104, preferably the coding region thereof, conferred the production of or the increase in phenylalanine compared with the wild type control.

The nucleic acid sequence of Sll0420 from Synechocystis sp., e.g. as shown in the respective line in column 5 of Table I, application no. 3, has been published in C. And the activity of the gene product thereof is the activity of urease subunit.

Accordingly, in one embodiment, the process of the present invention for producing tyrosine in a non-human organism, like a microorganism or a plant or a part thereof, comprises increasing or generating the activity of a gene product with the activity of a gene product conferring the activity of “urease subunit”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll0420, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll0420, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll0420, or a functional equivalent or a homolog thereof as depicted in column 8 of Table II, application no. 3, preferably a homolog or functional equivalent as depicted in column 8 of Table II B, application no. 3, and being depicted in the same respective line as said Sll0420, and preferably the activity is increased non-targeted.

Accordingly, in one embodiment, the molecule which activity is to be increased in the process of the invention is the gene product with an activity as a “urease subunit”, preferably it is the molecule of section (a) or (b) of this paragraph.

In particular, it was observed that in plants, especially in Arabidopsis thaliana, increasing or generating the activity of a gene product non-targeted with the activity of a “urease subunit”, preferably being encoded by a gene comprising the nucleic acid sequence SEQ ID NO. 50104, preferably the coding region thereof, conferred the production of or the increase in tyrosine compared with the wild type control.

The nucleic acid sequence of Sll0593 from Synechocystis sp., e.g. as shown in the respective line in column 5 of Table I, application no. 3, has been published in C. And the activity of the gene product thereof is the activity of glucokinase.

Accordingly, in one embodiment, the process of the present invention for producing citrulline in a non-human organism, like a microorganism or a plant or a part thereof, comprises increasing or generating the activity of a gene product with the activity of a gene product conferring the activity of “glucokinase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll0593, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll0593, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll0593, or a functional equivalent or a homolog thereof as depicted in column 8 of Table II, application no. 3, preferably a homolog or functional equivalent as depicted in column 8 of Table II B, application no. 3, and being depicted in the same respective line as said Sll0593, and preferably the activity is increased plastidic.

Accordingly, in one embodiment, the molecule which activity is to be increased in the process of the invention is the gene product with an activity as a “glucokinase”, preferably it is the molecule of section (a) or (b) of this paragraph.

In particular, it was observed that in plants, especially in Arabidopsis thaliana, increasing or generating the activity of a gene product plastidic with the activity of a “glucokinase”, preferably being encoded by a gene comprising the nucleic acid sequence SEQ ID NO. 85510, preferably the coding region thereof, conferred the production of or the increase in citrulline compared with the wild type control.

The nucleic acid sequence of Sll0682 from Synechocystis sp., e.g. as shown in the respective line in column 5 of Table I, application no. 3, has been published in C. And the activity of the gene product thereof is the activity of permease protein of phosphate ABC transporter.

Accordingly, in one embodiment, the process of the present invention for producing serine in a non-human organism, like a microorganism or a plant or a part thereof, comprises increasing or generating the activity of a gene product with the activity of a gene product conferring the activity of “permease protein of phosphate ABC transporter”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll0682, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll0682, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll0682, or a functional equivalent or a homolog thereof as depicted in column 8 of Table II, application no. 3, preferably a homolog or functional equivalent as depicted in column 8 of Table II B, application no. 3, and being depicted in the same respective line as said Sll0682, and preferably the activity is increased non-targeted.

Accordingly, in one embodiment, the molecule which activity is to be increased in the process of the invention is the gene product with an activity as a “permease protein of phosphate ABC transporter”, preferably it is the molecule of section (a) or (b) of this paragraph.

In particular, it was observed that in plants, especially in Arabidopsis thaliana, increasing or generating the activity of a gene product non-targeted with the activity of a “permease protein of phosphate ABC transporter”, preferably being encoded by a gene comprising the nucleic acid sequence SEQ ID NO. 50950, preferably the coding region thereof, conferred the production of or the increase in serine compared with the wild type control.

The nucleic acid sequence of Sll0682 from Synechocystis sp., e.g. as shown in the respective line in column 5 of Table I, application no. 3, has been published in C. And the activity of the gene product thereof is the activity of permease protein of phosphate ABC transporter.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “permease protein of phosphate ABC transporter”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll0682, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll0682, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll0682, or a functional equivalent or a homolog thereof as depicted in column 8 of Table II, application no. 3, preferably a homolog or functional equivalent as depicted in column 8 of Table II B, application no. 3, and being depicted in the same respective line as said Sll0682, and preferably the activity is increased non-targeted.

Accordingly, in one embodiment, the molecule which activity is to be increased in the process of the invention is the gene product with an activity as a “permease protein of phosphate ABC transporter”, preferably it is the molecule of section (a) or (b) of this paragraph.

In particular, it was observed that in plants, especially in Arabidopsis thaliana, increasing or generating the activity of a gene product non-targeted with the activity of a “permease protein of phosphate ABC transporter”, preferably being encoded by a gene comprising the nucleic acid sequence SEQ ID NO. 50950, preferably the coding region thereof, conferred the production of or the increase in 5-oxoproline compared with the wild type control.

The nucleic acid sequence of Sll0816 from Synechocystis sp., e.g. as shown in the respective line in column 5 of Table I, application no. 3, has been published in C. And the activity of the gene product thereof is the activity of oxireductase.

Accordingly, in one embodiment, the process of the present invention for producing aspartate in a non-human organism, like a microorganism or a plant or a part thereof, comprises increasing or generating the activity of a gene product with the activity of a gene product conferring the activity of “oxireductase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll0816, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll0816, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll0816, or a functional equivalent or a homolog thereof as depicted in column 8 of Table II, application no. 3, preferably a homolog or functional equivalent as depicted in column 8 of Table II B, application no. 3, and being depicted in the same respective line as said Sll0816, and preferably the activity is increased non-targeted.

Accordingly, in one embodiment, the molecule which activity is to be increased in the process of the invention is the gene product with an activity as a “oxireductase”, preferably it is the molecule of section (a) or (b) of this paragraph.

In particular, it was observed that in plants, especially in Arabidopsis thaliana, increasing or generating the activity of a gene product non-targeted with the activity of a “oxireductase”, preferably being encoded by a gene comprising the nucleic acid sequence SEQ ID NO. 51198, preferably the coding region thereof, conferred the production of or the increase in aspartate compared with the wild type control.

The nucleic acid sequence of Sll0816 from Synechocystis sp., e.g. as shown in the respective line in column 5 of Table I, application no. 3, has been published in C. And the activity of the gene product thereof is the activity of oxireductase.

Accordingly, in one embodiment, the process of the present invention for producing glycine in a non-human organism, like a microorganism or a plant or a part thereof, comprises increasing or generating the activity of a gene product with the activity of a gene product conferring the activity of “oxireductase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll0816, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll0816, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll0816, or a functional equivalent or a homolog thereof as depicted in column 8 of Table II, application no. 3, preferably a homolog or functional equivalent as depicted in column 8 of Table II B, application no. 3, and being depicted in the same respective line as said Sll0816, and preferably the activity is increased non-targeted.

Accordingly, in one embodiment, the molecule which activity is to be increased in the process of the invention is the gene product with an activity as a “oxireductase”, preferably it is the molecule of section (a) or (b) of this paragraph.

In particular, it was observed that in plants, especially in Arabidopsis thaliana, increasing or generating the activity of a gene product non-targeted with the activity of a “oxireductase”, preferably being encoded by a gene comprising the nucleic acid sequence SEQ ID NO. 51198, preferably the coding region thereof, conferred the production of or the increase in glycine compared with the wild type control.

The nucleic acid sequence of Sll0816 from Synechocystis sp., e.g. as shown in the respective line in column 5 of Table I, application no. 3, has been published in C. And the activity of the gene product thereof is the activity of oxireductase.

Accordingly, in one embodiment, the process of the present invention for producing serine in a non-human organism, like a microorganism or a plant or a part thereof, comprises increasing or generating the activity of a gene product with the activity of a gene product conferring the activity of “oxireductase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll0816, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll0816, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll0816, or a functional equivalent or a homolog thereof as depicted in column 8 of Table II, application no. 3, preferably a homolog or functional equivalent as depicted in column 8 of Table II B, application no. 3, and being depicted in the same respective line as said Sll0816, and preferably the activity is increased non-targeted.

Accordingly, in one embodiment, the molecule which activity is to be increased in the process of the invention is the gene product with an activity as a “oxireductase”, preferably it is the molecule of section (a) or (b) of this paragraph.

In particular, it was observed that in plants, especially in Arabidopsis thaliana, increasing or generating the activity of a gene product non-targeted with the activity of a “oxireductase”, preferably being encoded by a gene comprising the nucleic acid sequence SEQ ID NO. 51198, preferably the coding region thereof, conferred the production of or the increase in serine compared with the wild type control.

The nucleic acid sequence of Sll0816 from Synechocystis sp., e.g. as shown in the respective line in column 5 of Table I, application no. 3, has been published in C. And the activity of the gene product thereof is the activity of oxireductase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “oxireductase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll0816, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll0816, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll0816, or a functional equivalent or a homolog thereof as depicted in column 8 of Table II, application no. 3, preferably a homolog or functional equivalent as depicted in column 8 of Table II B, application no. 3, and being depicted in the same respective line as said Sll0816, and preferably the activity is increased non-targeted.

Accordingly, in one embodiment, the molecule which activity is to be increased in the process of the invention is the gene product with an activity as a “oxireductase”, preferably it is the molecule of section (a) or (b) of this paragraph.

In particular, it was observed that in plants, especially in Arabidopsis thaliana, increasing or generating the activity of a gene product non-targeted with the activity of a “oxireductase”, preferably being encoded by a gene comprising the nucleic acid sequence SEQ ID NO. 51198, preferably the coding region thereof, conferred the production of or the increase in tyrosine compared with the wild type control.

The nucleic acid sequence of Sll0891 from Synechocystis sp., e.g. as shown in the respective line in column 5 of Table I, application no. 3, has been published in C. And the activity of the gene product thereof is the activity of malate dehydrogenase.

Accordingly, in one embodiment, the process of the present invention for producing aspartate in a non-human organism, like a microorganism or a plant or a part thereof, comprises increasing or generating the activity of a gene product with the activity of a gene product conferring the activity of “malate dehydrogenase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll0891, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll0891, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll0891, or a functional equivalent or a homolog thereof as depicted in column 8 of Table II, application no. 3, preferably a homolog or functional equivalent as depicted in column 8 of Table II B, application no. 3, and being depicted in the same respective line as said Sll0891, and preferably the activity is increased plastidic.

Accordingly, in one embodiment, the molecule which activity is to be increased in the process of the invention is the gene product with an activity as a “malate dehydrogenase”, preferably it is the molecule of section (a) or (b) of this paragraph.

In particular, it was observed that in plants, especially in Arabidopsis thaliana, increasing or generating the activity of a gene product plastidic with the activity of a “malate dehydrogenase”, preferably being encoded by a gene comprising the nucleic acid sequence SEQ ID NO. 51268, preferably the coding region thereof, conferred the production of or the increase in aspartate compared with the wild type control.

The nucleic acid sequence of Sll0891 from Synechocystis sp., e.g. as shown in the respective line in column 5 of Table I, application no. 3, has been published in C. And the activity of the gene product thereof is the activity of malate dehydrogenase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “malate dehydrogenase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll0891, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll0891, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll0891, or a functional equivalent or a homolog thereof as depicted in column 8 of Table II, application no. 3, preferably a homolog or functional equivalent as depicted in column 8 of Table II B, application no. 3, and being depicted in the same respective line as said Sll0891, and preferably the activity is increased plastidic.

Accordingly, in one embodiment, the molecule which activity is to be increased in the process of the invention is the gene product with an activity as a “malate dehydrogenase”, preferably it is the molecule of section (a) or (b) of this paragraph.

In particular, it was observed that in plants, especially in Arabidopsis thaliana, increasing or generating the activity of a gene product plastidic with the activity of a “malate dehydrogenase”, preferably being encoded by a gene comprising the nucleic acid sequence SEQ ID NO. 51268, preferably the coding region thereof, conferred the production of or the increase in phenylalanine compared with the wild type control.

The nucleic acid sequence of Sll0891 from Synechocystis sp., e.g. as shown in the respective line in column 5 of Table I, application no. 3, has been published in C. And the activity of the gene product thereof is the activity of malate dehydrogenase.

Accordingly, in one embodiment, the process of the present invention for producing serine in a non-human organism, like a microorganism or a plant or a part thereof, comprises increasing or generating the activity of a gene product with the activity of a gene product conferring the activity of “malate dehydrogenase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll0891, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll0891, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll0891, or a functional equivalent or a homolog thereof as depicted in column 8 of Table II, application no. 3, preferably a homolog or functional equivalent as depicted in column 8 of Table II B, application no. 3, and being depicted in the same respective line as said Sll0891, and preferably the activity is increased plastidic.

Accordingly, in one embodiment, the molecule which activity is to be increased in the process of the invention is the gene product with an activity as a “malate dehydrogenase”, preferably it is the molecule of section (a) or (b) of this paragraph.

In particular, it was observed that in plants, especially in Arabidopsis thaliana, increasing or generating the activity of a gene product plastidic with the activity of a “malate dehydrogenase”, preferably being encoded by a gene comprising the nucleic acid sequence SEQ ID NO. 51268, preferably the coding region thereof, conferred the production of or the increase in serine compared with the wild type control.

The nucleic acid sequence of Sll0934 from Synechocystis sp., e.g. as shown in the respective line in column 5 of Table I, application no. 3, has been published in C. And the activity of the gene product thereof is the activity of 3-deoxy-7-phosphoheptulonate synthase.

Accordingly, in one embodiment, the process of the present invention for producing aspartate in a non-human organism, like a microorganism or a plant or a part thereof, comprises increasing or generating the activity of a gene product with the activity of a gene product conferring the activity of “3-deoxy-7-phosphoheptulonate synthase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll0934, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll0934, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll0934, or a functional equivalent or a homolog thereof as depicted in column 8 of Table II, application no. 3, preferably a homolog or functional equivalent as depicted in column 8 of Table II B, application no. 3, and being depicted in the same respective line as said Sll0934, and preferably the activity is increased mitochondrial.

Accordingly, in one embodiment, the molecule which activity is to be increased in the process of the invention is the gene product with an activity as a “3-deoxy-7-phosphoheptulonate synthase”, preferably it is the molecule of section (a) or (b) of this paragraph.

In particular, it was observed that in plants, especially in Arabidopsis thaliana, increasing or generating the activity of a gene product mitochondrial with the activity of a “3-deoxy-7-phosphoheptulonate synthase”, preferably being encoded by a gene comprising the nucleic acid sequence SEQ ID NO. 52246, preferably the coding region thereof, conferred the production of or the increase in aspartate compared with the wild type control.

The nucleic acid sequence of Sll0945 from Synechocystis sp., e.g. as shown in the respective line in column 5 of Table I, application no. 3, has been published in C. And the activity of the gene product thereof is the activity of glycogen synthase.

Accordingly, in one embodiment, the process of the present invention for producing serine in a non-human organism, like a microorganism or a plant or a part thereof, comprises increasing or generating the activity of a gene product with the activity of a gene product conferring the activity of “glycogen synthase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll0945, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll0945, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll0945, or a functional equivalent or a homolog thereof as depicted in column 8 of Table II, application no. 3, preferably a homolog or functional equivalent as depicted in column 8 of Table II B, application no. 3, and being depicted in the same respective line as said Sll0945, and preferably the activity is increased non-targeted.

Accordingly, in one embodiment, the molecule which activity is to be increased in the process of the invention is the gene product with an activity as a “glycogen synthase”, preferably it is the molecule of section (a) or (b) of this paragraph.

In particular, it was observed that in plants, especially in Arabidopsis thaliana, increasing or generating the activity of a gene product non-targeted with the activity of a “glycogen synthase”, preferably being encoded by a gene comprising the nucleic acid sequence SEQ ID NO. 52364, preferably the coding region thereof, conferred the production of or the increase in serine compared with the wild type control.

The nucleic acid sequence of Sll1031 from Synechocystis sp., e.g. as shown in the respective line in column 5 of Table I, application no. 3, has been published in C. And the activity of the gene product thereof is the activity of carbon dioxide concentrating mechanism protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “carbon dioxide concentrating mechanism protein”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1031, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1031, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1031, or a functional equivalent or a homolog thereof as depicted in column 8 of Table II, application no. 3, preferably a homolog or functional equivalent as depicted in column 8 of Table II B, application no. 3, and being depicted in the same respective line as said Sll1031, and preferably the activity is increased mitochondrial.

Accordingly, in one embodiment, the molecule which activity is to be increased in the process of the invention is the gene product with an activity as a “carbon dioxide concentrating mechanism protein”, preferably it is the molecule of section (a) or (b) of this paragraph.

In particular, it was observed that in plants, especially in Arabidopsis thaliana, increasing or generating the activity of a gene product mitochondrial with the activity of a “carbon dioxide concentrating mechanism protein”, preferably being encoded by a gene comprising the nucleic acid sequence SEQ ID NO. 52634, preferably the coding region thereof, conferred the production of or the increase in aspartate compared with the wild type control.

The nucleic acid sequence of Sll1031 from Synechocystis sp., e.g. as shown in the respective line in column 5 of Table I, application no. 3, has been published in C. And the activity of the gene product thereof is the activity of carbon dioxide concentrating mechanism protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “carbon dioxide concentrating mechanism protein”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1031, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1031, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1031, or a functional equivalent or a homolog thereof as depicted in column 8 of Table II, application no. 3, preferably a homolog or functional equivalent as depicted in column 8 of Table II B, application no. 3, and being depicted in the same respective line as said Sll1031, and preferably the activity is increased mitochondrial.

Accordingly, in one embodiment, the molecule which activity is to be increased in the process of the invention is the gene product with an activity as a “carbon dioxide concentrating mechanism protein”, preferably it is the molecule of section (a) or (b) of this paragraph.

In particular, it was observed that in plants, especially in Arabidopsis thaliana, increasing or generating the activity of a gene product mitochondrial with the activity of a “carbon dioxide concentrating mechanism protein”, preferably being encoded by a gene comprising the nucleic acid sequence SEQ ID NO. 52634, preferably the coding region thereof, conferred the production of or the increase in phenylalanine compared with the wild type control.

The nucleic acid sequence of Sll1031 from Synechocystis sp., e.g. as shown in the respective line in column 5 of Table I, application no. 3, has been published in C. And the activity of the gene product thereof is the activity of carbon dioxide concentrating mechanism protein.

Accordingly, in one embodiment, the process of the present invention for producing serine in a non-human organism, like a microorganism or a plant or a part thereof, comprises increasing or generating the activity of a gene product with the activity of a gene product conferring the activity of “carbon dioxide concentrating mechanism protein”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1031, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1031, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1031, or a functional equivalent or a homolog thereof as depicted in column 8 of Table II, application no. 3, preferably a homolog or functional equivalent as depicted in column 8 of Table II B, application no. 3, and being depicted in the same respective line as said Sll1031, and preferably the activity is increased mitochondrial.

Accordingly, in one embodiment, the molecule which activity is to be increased in the process of the invention is the gene product with an activity as a “carbon dioxide concentrating mechanism protein”, preferably it is the molecule of section (a) or (b) of this paragraph.

In particular, it was observed that in plants, especially in Arabidopsis thaliana, increasing or generating the activity of a gene product mitochondrial with the activity of a “carbon dioxide concentrating mechanism protein”, preferably being encoded by a gene comprising the nucleic acid sequence SEQ ID NO. 52634, preferably the coding region thereof, conferred the production of or the increase in serine compared with the wild type control.

The nucleic acid sequence of Sll1032 from Synechocystis sp., e.g. as shown in the respective line in column 5 of Table I, application no. 3, has been published in C. And the activity of the gene product thereof is the activity of sll1032-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “sll1032-protein”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1032, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1032, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1032, or a functional equivalent or a homolog thereof as depicted in column 8 of Table II, application no. 3, preferably a homolog or functional equivalent as depicted in column 8 of Table II B, application no. 3, and being depicted in the same respective line as said Sll1032, and preferably the activity is increased mitochondrial.

Accordingly, in one embodiment, the molecule which activity is to be increased in the process of the invention is the gene product with an activity as a “sll1032-protein”, preferably it is the molecule of section (a) or (b) of this paragraph.

In particular, it was observed that in plants, especially in Arabidopsis thaliana, increasing or generating the activity of a gene product mitochondrial with the activity of a “sll1032-protein”, preferably being encoded by a gene comprising the nucleic acid sequence SEQ ID NO. 85723, preferably the coding region thereof, conferred the production of or the increase in aspartate compared with the wild type control.

The nucleic acid sequence of Sll1056 from Synechocystis sp., e.g. as shown in the respective line in column 5 of Table I, application no. 3, has been published in C. And the activity of the gene product thereof is the activity of phosphoribosylformyl glycinamidine synthase subunit.

Accordingly, in one embodiment, the process of the present invention for producing serine in a non-human organism, like a microorganism or a plant or a part thereof, comprises increasing or generating the activity of a gene product with the activity of a gene product conferring the activity of “phosphoribosylformyl glycinamidine synthase subunit”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1056, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1056, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1056, or a functional equivalent or a homolog thereof as depicted in column 8 of Table II, application no. 3, preferably a homolog or functional equivalent as depicted in column 8 of Table II B, application no. 3, and being depicted in the same respective line as said Sll1056, and preferably the activity is increased plastidic.

Accordingly, in one embodiment, the molecule which activity is to be increased in the process of the invention is the gene product with an activity as a “phosphoribosylformyl glycinamidine synthase subunit”, preferably it is the molecule of section (a) or (b) of this paragraph.

In particular, it was observed that in plants, especially in Arabidopsis thaliana, increasing or generating the activity of a gene product plastidic with the activity of a “phosphoribosylformyl glycinamidine synthase subunit”, preferably being encoded by a gene comprising the nucleic acid sequence SEQ ID NO. 52660, preferably the coding region thereof, conferred the production of or the increase in serine compared with the wild type control.

The nucleic acid sequence of Sll1108 from Synechocystis sp., e.g. as shown in the respective line in column 5 of Table I, application no. 3, has been published in C. And the activity of the gene product thereof is the activity of 5′-nucleotidase.

Accordingly, in one embodiment, the process of the present invention for producing aspartate in a non-human organism, like a microorganism or a plant or a part thereof, comprises increasing or generating the activity of a gene product with the activity of a gene product conferring the activity of “5′-nucleotidase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1108, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1108, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1108, or a functional equivalent or a homolog thereof as depicted in column 8 of Table II, application no. 3, preferably a homolog or functional equivalent as depicted in column 8 of Table II B, application no. 3, and being depicted in the same respective line as said Sll1108, and preferably the activity is increased mitochondrial.

Accordingly, in one embodiment, the molecule which activity is to be increased in the process of the invention is the gene product with an activity as a “5′-nucleotidase”, preferably it is the molecule of section (a) or (b) of this paragraph.

In particular, it was observed that in plants, especially in Arabidopsis thaliana, increasing or generating the activity of a gene product mitochondrial with the activity of a “5′-nucleotidase”, preferably being encoded by a gene comprising the nucleic acid sequence SEQ ID NO. 11211, preferably the coding region thereof, conferred the production of or the increase in aspartate compared with the wild type control.

The nucleic acid sequence of Sll1108 from Synechocystis sp., e.g. as shown in the respective line in column 5 of Table I, application no. 3, has been published in C. And the activity of the gene product thereof is the activity of 5′-nucleotidase.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “5′-nucleotidase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1108, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1108, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1108, or a functional equivalent or a homolog thereof as depicted in column 8 of Table II, application no. 3, preferably a homolog or functional equivalent as depicted in column 8 of Table II B, application no. 3, and being depicted in the same respective line as said Sll1108, and preferably the activity is increased mitochondrial.

Accordingly, in one embodiment, the molecule which activity is to be increased in the process of the invention is the gene product with an activity as a “5′-nucleotidase”, preferably it is the molecule of section (a) or (b) of this paragraph.

In particular, it was observed that in plants, especially in Arabidopsis thaliana, increasing or generating the activity of a gene product mitochondrial with the activity of a “5′-nucleotidase”, preferably being encoded by a gene comprising the nucleic acid sequence SEQ ID NO. 11211, preferably the coding region thereof, conferred the production of or the increase in 5-oxoproline compared with the wild type control.

The nucleic acid sequence of Sll1393 from Synechocystis sp., e.g. as shown in the respective line in column 5 of Table I, application no. 3, has been published in C. And the activity of the gene product thereof is the activity of glycogen (starch) synthase.

Accordingly, in one embodiment, the process of the present invention for producing asparagine in a non-human organism, like a microorganism or a plant or a part thereof, comprises increasing or generating the activity of a gene product with the activity of a gene product conferring the activity of “glycogen (starch) synthase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1393, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1393, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1393, or a functional equivalent or a homolog thereof as depicted in column 8 of Table II, application no. 3, preferably a homolog or functional equivalent as depicted in column 8 of Table II B, application no. 3, and being depicted in the same respective line as said Sll1393, and preferably the activity is increased non-targeted.

Accordingly, in one embodiment, the molecule which activity is to be increased in the process of the invention is the gene product with an activity as a “glycogen (starch) synthase”, preferably it is the molecule of section (a) or (b) of this paragraph.

In particular, it was observed that in plants, especially in Arabidopsis thaliana, increasing or generating the activity of a gene product non-targeted with the activity of a “glycogen (starch) synthase”, preferably being encoded by a gene comprising the nucleic acid sequence SEQ ID NO. 53456, preferably the coding region thereof, conferred the production of or the increase in asparagine compared with the wild type control.

The nucleic acid sequence of Sll1393 from Synechocystis sp., e.g. as shown in the respective line in column 5 of Table I, application no. 3, has been published in C. And the activity of the gene product thereof is the activity of glycogen (starch) synthase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “glycogen (starch) synthase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1393, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1393, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1393, or a functional equivalent or a homolog thereof as depicted in column 8 of Table II, application no. 3, preferably a homolog or functional equivalent as depicted in column 8 of Table II B, application no. 3, and being depicted in the same respective line as said Sll1393, and preferably the activity is increased non-targeted.

Accordingly, in one embodiment, the molecule which activity is to be increased in the process of the invention is the gene product with an activity as a “glycogen (starch) synthase”, preferably it is the molecule of section (a) or (b) of this paragraph.

In particular, it was observed that in plants, especially in Arabidopsis thaliana, increasing or generating the activity of a gene product non-targeted with the activity of a “glycogen (starch) synthase”, preferably being encoded by a gene comprising the nucleic acid sequence SEQ ID NO. 53456, preferably the coding region thereof, conferred the production of or the increase in tyrosine compared with the wild type control.

The nucleic acid sequence of Sll1443 from Synechocystis sp., e.g. as shown in the respective line in column 5 of Table I, application no. 3, has been published in C. And the activity of the gene product thereof is the activity of CTP synthetase.

Accordingly, in one embodiment, the process of the present invention for producing aspartate in a non-human organism, like a microorganism or a plant or a part thereof, comprises increasing or generating the activity of a gene product with the activity of a gene product conferring the activity of “CTP synthetase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1443, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1443, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1443, or a functional equivalent or a homolog thereof as depicted in column 8 of Table II, application no. 3, preferably a homolog or functional equivalent as depicted in column 8 of Table II B, application no. 3, and being depicted in the same respective line as said Sll1443, and preferably the activity is increased mitochondrial.

Accordingly, in one embodiment, the molecule which activity is to be increased in the process of the invention is the gene product with an activity as a “CTP synthetase”, preferably it is the molecule of section (a) or (b) of this paragraph.

In particular, it was observed that in plants, especially in Arabidopsis thaliana, increasing or generating the activity of a gene product mitochondrial with the activity of a “CTP synthetase”, preferably being encoded by a gene comprising the nucleic acid sequence SEQ ID NO. 53878, preferably the coding region thereof, conferred the production of or the increase in aspartate compared with the wild type control.

The nucleic acid sequence of Sll1450 from Synechocystis sp., e.g. as shown in the respective line in column 5 of Table I, application no. 3, has been published in C. And the activity of the gene product thereof is the activity of nitrate/nitrite transport protein.

Accordingly, in one embodiment, the process of the present invention for producing glycine in a non-human organism, like a microorganism or a plant or a part thereof, comprises increasing or generating the activity of a gene product with the activity of a gene product conferring the activity of “nitrate/nitrite transport protein”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1450, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1450, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1450, or a functional equivalent or a homolog thereof as depicted in column 8 of Table II, application no. 3, preferably a homolog or functional equivalent as depicted in column 8 of Table II B, application no. 3, and being depicted in the same respective line as said Sll1450, and preferably the activity is increased mitochondrial.

Accordingly, in one embodiment, the molecule which activity is to be increased in the process of the invention is the gene product with an activity as a “nitrate/nitrite transport protein”, preferably it is the molecule of section (a) or (b) of this paragraph.

In particular, it was observed that in plants, especially in Arabidopsis thaliana, increasing or generating the activity of a gene product mitochondrial with the activity of a “nitrate/nitrite transport protein”, preferably being encoded by a gene comprising the nucleic acid sequence SEQ ID NO. 54337, preferably the coding region thereof, conferred the production of or the increase in glycine compared with the wild type control.

The nucleic acid sequence of Sll1450 from Synechocystis sp., e.g. as shown in the respective line in column 5 of Table I, application no. 3, has been published in C. And the activity of the gene product thereof is the activity of nitrate/nitrite transport protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “nitrate/nitrite transport protein”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1450, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1450, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1450, or a functional equivalent or a homolog thereof as depicted in column 8 of Table II, application no. 3, preferably a homolog or functional equivalent as depicted in column 8 of Table II B, application no. 3, and being depicted in the same respective line as said Sll1450, and preferably the activity is increased mitochondrial.

Accordingly, in one embodiment, the molecule which activity is to be increased in the process of the invention is the gene product with an activity as a “nitrate/nitrite transport protein”, preferably it is the molecule of section (a) or (b) of this paragraph.

In particular, it was observed that in plants, especially in Arabidopsis thaliana, increasing or generating the activity of a gene product mitochondrial with the activity of a “nitrate/nitrite transport protein”, preferably being encoded by a gene comprising the nucleic acid sequence SEQ ID NO. 54337, preferably the coding region thereof, conferred the production of or the increase in phenylalanine compared with the wild type control.

The nucleic acid sequence of Sll1450 from Synechocystis sp., e.g. as shown in the respective line in column 5 of Table I, application no. 3, has been published in C. And the activity of the gene product thereof is the activity of nitrate/nitrite transport protein.

Accordingly, in one embodiment, the process of the present invention for producing serine in a non-human organism, like a microorganism or a plant or a part thereof, comprises increasing or generating the activity of a gene product with the activity of a gene product conferring the activity of “nitrate/nitrite transport protein”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1450, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1450, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1450, or a functional equivalent or a homolog thereof as depicted in column 8 of Table II, application no. 3, preferably a homolog or functional equivalent as depicted in column 8 of Table II B, application no. 3, and being depicted in the same respective line as said Sll1450, and preferably the activity is increased mitochondrial.

Accordingly, in one embodiment, the molecule which activity is to be increased in the process of the invention is the gene product with an activity as a “nitrate/nitrite transport protein”, preferably it is the molecule of section (a) or (b) of this paragraph.

In particular, it was observed that in plants, especially in Arabidopsis thaliana, increasing or generating the activity of a gene product mitochondrial with the activity of a “nitrate/nitrite transport protein”, preferably being encoded by a gene comprising the nucleic acid sequence SEQ ID NO. 54337, preferably the coding region thereof, conferred the production of or the increase in serine compared with the wild type control.

The nucleic acid sequence of Sll1450 from Synechocystis sp., e.g. as shown in the respective line in column 5 of Table I, application no. 3, has been published in C. And the activity of the gene product thereof is the activity of nitrate/nitrite transport protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “nitrate/nitrite transport protein”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1450, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1450, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1450, or a functional equivalent or a homolog thereof as depicted in column 8 of Table II, application no. 3, preferably a homolog or functional equivalent as depicted in column 8 of Table II B, application no. 3, and being depicted in the same respective line as said Sll1450, and preferably the activity is increased mitochondrial.

Accordingly, in one embodiment, the molecule which activity is to be increased in the process of the invention is the gene product with an activity as a “nitrate/nitrite transport protein”, preferably it is the molecule of section (a) or (b) of this paragraph.

In particular, it was observed that in plants, especially in Arabidopsis thaliana, increasing or generating the activity of a gene product mitochondrial with the activity of a “nitrate/nitrite transport protein”, preferably being encoded by a gene comprising the nucleic acid sequence SEQ ID NO. 54337, preferably the coding region thereof, conferred the production of or the increase in tyrosine compared with the wild type control.

The nucleic acid sequence of Sll1522 from Synechocystis sp., e.g. as shown in the respective line in column 5 of Table I, application no. 3, has been published in C. And the activity of the gene product thereof is the activity of CDP-diacylglycerol-glycerol-3-phosphate 3-phosphatidyltransferase.

Accordingly, in one embodiment, the process of the present invention for producing aspartate in a non-human organism, like a microorganism or a plant or a part thereof, comprises increasing or generating the activity of a gene product with the activity of a gene product conferring the activity of “CDP-diacylglycerol-glycerol-3-phosphate 3-phosphatidyltransferase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1522, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1522, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1522, or a functional equivalent or a homolog thereof as depicted in column 8 of Table II, application no. 3, preferably a homolog or functional equivalent as depicted in column 8 of Table II B, application no. 3, and being depicted in the same respective line as said Sll1522, and preferably the activity is increased plastidic.

Accordingly, in one embodiment, the molecule which activity is to be increased in the process of the invention is the gene product with an activity as a “CDP-diacylglycerol-glycerol-3-phosphate 3-phosphatidyltransferase”, preferably it is the molecule of section (a) or (b) of this paragraph.

In particular, it was observed that in plants, especially in Arabidopsis thaliana, increasing or generating the activity of a gene product plastidic with the activity of a “CDP-diacylglycerol-glycerol-3-phosphate 3-phosphatidyltransferase”, preferably being encoded by a gene comprising the nucleic acid sequence SEQ ID NO. 54452, preferably the coding region thereof, conferred the production of or the increase in aspartate compared with the wild type control.

The nucleic acid sequence of Sll1522 from Synechocystis sp., e.g. as shown in the respective line in column 5 of Table I, application no. 3, has been published in C. And the activity of the gene product thereof is the activity of CDP-diacylglycerol-glycerol-3-phosphate 3-phosphatidyltransferase.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “CDP-diacylglycerol-glycerol-3-phosphate 3-phosphatidyltransferase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1522, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1522, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1522, or a functional equivalent or a homolog thereof as depicted in column 8 of Table II, application no. 3, preferably a homolog or functional equivalent as depicted in column 8 of Table II B, application no. 3, and being depicted in the same respective line as said Sll1522, and preferably the activity is increased plastidic.

Accordingly, in one embodiment, the molecule which activity is to be increased in the process of the invention is the gene product with an activity as a “CDP-diacylglycerol-glycerol-3-phosphate 3-phosphatidyltransferase”, preferably it is the molecule of section (a) or (b) of this paragraph.

In particular, it was observed that in plants, especially in Arabidopsis thaliana, increasing or generating the activity of a gene product plastidic with the activity of a “CDP-diacylglycerol-glycerol-3-phosphate 3-phosphatidyltransferase”, preferably being encoded by a gene comprising the nucleic acid sequence SEQ ID NO. 54452, preferably the coding region thereof, conferred the production of or the increase in 5-oxoproline compared with the wild type control.

The nucleic acid sequence of Sll1545 from Synechocystis sp., e.g. as shown in the respective line in column 5 of Table I, application no. 3, has been published in C. And the activity of the gene product thereof is the activity of glutathione S-transferase.

Accordingly, in one embodiment, the process of the present invention for producing serine in a non-human organism, like a microorganism or a plant or a part thereof, comprises increasing or generating the activity of a gene product with the activity of a gene product conferring the activity of “glutathione S-transferase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1545, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1545, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1545, or a functional equivalent or a homolog thereof as depicted in column 8 of Table II, application no. 3, preferably a homolog or functional equivalent as depicted in column 8 of Table II B, application no. 3, and being depicted in the same respective line as said Sll1545, and preferably the activity is increased mitochondrial.

Accordingly, in one embodiment, the molecule which activity is to be increased in the process of the invention is the gene product with an activity as a “glutathione S-transferase”, preferably it is the molecule of section (a) or (b) of this paragraph.

In particular, it was observed that in plants, especially in Arabidopsis thaliana, increasing or generating the activity of a gene product mitochondrial with the activity of a “glutathione S-transferase”, preferably being encoded by a gene comprising the nucleic acid sequence SEQ ID NO. 11423, preferably the coding region thereof, conferred the production of or the increase in serine compared with the wild type control.

The nucleic acid sequence of Sll1546 from Synechocystis sp., e.g. as shown in the respective line in column 5 of Table I, application no. 3, has been published in C. And the activity of the gene product thereof is the activity of exopolyphosphatase.

Accordingly, in one embodiment, the process of the present invention for producing aspartate in a non-human organism, like a microorganism or a plant or a part thereof, comprises increasing or generating the activity of a gene product with the activity of a gene product conferring the activity of “exopolyphosphatase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1546, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1546, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1546, or a functional equivalent or a homolog thereof as depicted in column 8 of Table II, application no. 3, preferably a homolog or functional equivalent as depicted in column 8 of Table II B, application no. 3, and being depicted in the same respective line as said Sll1546, and preferably the activity is increased non-targeted.

Accordingly, in one embodiment, the molecule which activity is to be increased in the process of the invention is the gene product with an activity as a “exopolyphosphatase”, preferably it is the molecule of section (a) or (b) of this paragraph.

In particular, it was observed that in plants, especially in Arabidopsis thaliana, increasing or generating the activity of a gene product non-targeted with the activity of a “exopolyphosphatase”, preferably being encoded by a gene comprising the nucleic acid sequence SEQ ID NO. 54804, preferably the coding region thereof, conferred the production of or the increase in aspartate compared with the wild type control.

The nucleic acid sequence of Sll1546 from Synechocystis sp., e.g. as shown in the respective line in column 5 of Table I, application no. 3, has been published in C. And the activity of the gene product thereof is the activity of exopolyphosphatase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “exopolyphosphatase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1546, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1546, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1546, or a functional equivalent or a homolog thereof as depicted in column 8 of Table II, application no. 3, preferably a homolog or functional equivalent as depicted in column 8 of Table II B, application no. 3, and being depicted in the same respective line as said Sll1546, and preferably the activity is increased non-targeted.

Accordingly, in one embodiment, the molecule which activity is to be increased in the process of the invention is the gene product with an activity as a “exopolyphosphatase”, preferably it is the molecule of section (a) or (b) of this paragraph.

In particular, it was observed that in plants, especially in Arabidopsis thaliana, increasing or generating the activity of a gene product non-targeted with the activity of a “exopolyphosphatase”, preferably being encoded by a gene comprising the nucleic acid sequence SEQ ID NO. 54804, preferably the coding region thereof, conferred the production of or the increase in phenylalanine compared with the wild type control.

The nucleic acid sequence of Sll1546 from Synechocystis sp., e.g. as shown in the respective line in column 5 of Table I, application no. 3, has been published in C. And the activity of the gene product thereof is the activity of exopolyphosphatase.

Accordingly, in one embodiment, the process of the present invention for producing serine in a non-human organism, like a microorganism or a plant or a part thereof, comprises increasing or generating the activity of a gene product with the activity of a gene product conferring the activity of “exopolyphosphatase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1546, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1546, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1546, or a functional equivalent or a homolog thereof as depicted in column 8 of Table II, application no. 3, preferably a homolog or functional equivalent as depicted in column 8 of Table II B, application no. 3, and being depicted in the same respective line as said Sll1546, and preferably the activity is increased non-targeted.

Accordingly, in one embodiment, the molecule which activity is to be increased in the process of the invention is the gene product with an activity as a “exopolyphosphatase”, preferably it is the molecule of section (a) or (b) of this paragraph.

In particular, it was observed that in plants, especially in Arabidopsis thaliana, increasing or generating the activity of a gene product non-targeted with the activity of a “exopolyphosphatase”, preferably being encoded by a gene comprising the nucleic acid sequence SEQ ID NO. 54804, preferably the coding region thereof, conferred the production of or the increase in serine compared with the wild type control.

The nucleic acid sequence of Sll1598 from Synechocystis sp., e.g. as shown in the respective line in column 5 of Table I, application no. 3, has been published in C. And the activity of the gene product thereof is the activity of ABC metal ion transporter substrate-binding protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “ABC metal ion transporter substrate-binding protein”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1598, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1598, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1598, or a functional equivalent or a homolog thereof as depicted in column 8 of Table II, application no. 3, preferably a homolog or functional equivalent as depicted in column 8 of Table II B, application no. 3, and being depicted in the same respective line as said Sll1598, and preferably the activity is increased mitochondrial.

Accordingly, in one embodiment, the molecule which activity is to be increased in the process of the invention is the gene product with an activity as a “ABC metal ion transporter substratebinding protein”, preferably it is the molecule of section (a) or (b) of this paragraph.

In particular, it was observed that in plants, especially in Arabidopsis thaliana, increasing or generating the activity of a gene product mitochondrial with the activity of a “ABC metal ion transporter substrate-binding protein”, preferably being encoded by a gene comprising the nucleic acid sequence SEQ ID NO. 85743, preferably the coding region thereof, conferred the production of or the increase in aspartate compared with the wild type control.

The nucleic acid sequence of Sll1598 from Synechocystis sp., e.g. as shown in the respective line in column 5 of Table I, application no. 3, has been published in C. And the activity of the gene product thereof is the activity of ABC metal ion transporter substrate-binding protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “ABC metal ion transporter substrate-binding protein”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1598, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1598, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1598, or a functional equivalent or a homolog thereof as depicted in column 8 of Table II, application no. 3, preferably a homolog or functional equivalent as depicted in column 8 of Table II B, application no. 3, and being depicted in the same respective line as said Sll1598, and preferably the activity is increased mitochondrial.

Accordingly, in one embodiment, the molecule which activity is to be increased in the process of the invention is the gene product with an activity as a “ABC metal ion transporter substratebinding protein”, preferably it is the molecule of section (a) or (b) of this paragraph.

In particular, it was observed that in plants, especially in Arabidopsis thaliana, increasing or generating the activity of a gene product mitochondrial with the activity of a “ABC metal ion transporter substrate-binding protein”, preferably being encoded by a gene comprising the nucleic acid sequence SEQ ID NO. 85743, preferably the coding region thereof, conferred the production of or the increase in phenylalanine compared with the wild type control.

The nucleic acid sequence of Sll1761 from Synechocystis sp., e.g. as shown in the respective line in column 5 of Table I, application no. 3, has been published in C. And the activity of the gene product thereof is the activity of sll1761-protein.

Accordingly, in one embodiment, the process of the present invention for producing serine in a non-human organism, like a microorganism or a plant or a part thereof, comprises increasing or generating the activity of a gene product with the activity of a gene product conferring the activity of “sll1761-protein”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1761, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1761, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1761, or a functional equivalent or a homolog thereof as depicted in column 8 of Table II, application no. 3, preferably a homolog or functional equivalent as depicted in column 8 of Table II B, application no. 3, and being depicted in the same respective line as said Sll1761, and preferably the activity is increased non-targeted.

Accordingly, in one embodiment, the molecule which activity is to be increased in the process of the invention is the gene product with an activity as a “sll1761-protein”, preferably it is the molecule of section (a) or (b) of this paragraph.

In particular, it was observed that in plants, especially in Arabidopsis thaliana, increasing or generating the activity of a gene product non-targeted with the activity of a “sll1761-protein”, preferably being encoded by a gene comprising the nucleic acid sequence SEQ ID NO. 55379, preferably the coding region thereof, conferred the production of or the increase in serine compared with the wild type control.

The nucleic acid sequence of Sll1761 from Synechocystis sp., e.g. as shown in the respective line in column 5 of Table I, application no. 3, has been published in C. And the activity of the gene product thereof is the activity of sll1761-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “sll1761-protein”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1761, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1761, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1761, or a functional equivalent or a homolog thereof as depicted in column 8 of Table II, application no. 3, preferably a homolog or functional equivalent as depicted in column 8 of Table II B, application no. 3, and being depicted in the same respective line as said Sll1761, and preferably the activity is increased non-targeted.

Accordingly, in one embodiment, the molecule which activity is to be increased in the process of the invention is the gene product with an activity as a “sll1761-protein”, preferably it is the molecule of section (a) or (b) of this paragraph.

In particular, it was observed that in plants, especially in Arabidopsis thaliana, increasing or generating the activity of a gene product non-targeted with the activity of a “sll1761-protein”, preferably being encoded by a gene comprising the nucleic acid sequence SEQ ID NO. 55379, preferably the coding region thereof, conferred the production of or the increase in tyrosine compared with the wild type control.

The nucleic acid sequence of Sll1815 from Synechocystis sp., e.g. as shown in the respective line in column 5 of Table I, application no. 3, has been published in C. And the activity of the gene product thereof is the activity of adenylate kinase.

Accordingly, in one embodiment, the process of the present invention for producing alanine in a non-human organism, like a microorganism or a plant or a part thereof, comprises increasing or generating the activity of a gene product with the activity of a gene product conferring the activity of “adenylate kinase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1815, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1815, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1815, or a functional equivalent or a homolog thereof as depicted in column 8 of Table II, application no. 3, preferably a homolog or functional equivalent as depicted in column 8 of Table II B, application no. 3, and being depicted in the same respective line as said Sll1815, and preferably the activity is increased mitochondrial.

Accordingly, in one embodiment, the molecule which activity is to be increased in the process of the invention is the gene product with an activity as a “adenylate kinase”, preferably it is the molecule of section (a) or (b) of this paragraph.

In particular, it was observed that in plants, especially in Arabidopsis thaliana, increasing or generating the activity of a gene product mitochondrial with the activity of a “adenylate kinase”, preferably being encoded by a gene comprising the nucleic acid sequence SEQ ID NO. 85808, preferably the coding region thereof, conferred the production of or the increase in alanine compared with the wild type control.

The nucleic acid sequence of Sll1815 from Synechocystis sp., e.g. as shown in the respective line in column 5 of Table I, application no. 3, has been published in C. And the activity of the gene product thereof is the activity of adenylate kinase.

Accordingly, in one embodiment, the process of the present invention for producing citrulline in a non-human organism, like a microorganism or a plant or a part thereof, comprises increasing or generating the activity of a gene product with the activity of a gene product conferring the activity of “adenylate kinase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1815, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1815, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1815, or a functional equivalent or a homolog thereof as depicted in column 8 of Table II, application no. 3, preferably a homolog or functional equivalent as depicted in column 8 of Table II B, application no. 3, and being depicted in the same respective line as said Sll1815, and preferably the activity is increased mitochondrial.

Accordingly, in one embodiment, the molecule which activity is to be increased in the process of the invention is the gene product with an activity as a “adenylate kinase”, preferably it is the molecule of section (a) or (b) of this paragraph.

In particular, it was observed that in plants, especially in Arabidopsis thaliana, increasing or generating the activity of a gene product mitochondrial with the activity of a “adenylate kinase”, preferably being encoded by a gene comprising the nucleic acid sequence SEQ ID NO. 85808, preferably the coding region thereof, conferred the production of or the increase in citrulline compared with the wild type control.

The nucleic acid sequence of Sll1815 from Synechocystis sp., e.g. as shown in the respective line in column 5 of Table I, application no. 3, has been published in C. And the activity of the gene product thereof is the activity of adenylate kinase.

Accordingly, in one embodiment, the process of the present invention for producing glycine in a non-human organism, like a microorganism or a plant or a part thereof, comprises increasing or generating the activity of a gene product with the activity of a gene product conferring the activity of “adenylate kinase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1815, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1815, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1815, or a functional equivalent or a homolog thereof as depicted in column 8 of Table II, application no. 3, preferably a homolog or functional equivalent as depicted in column 8 of Table II B, application no. 3, and being depicted in the same respective line as said Sll1815, and preferably the activity is increased mitochondrial.

Accordingly, in one embodiment, the molecule which activity is to be increased in the process of the invention is the gene product with an activity as a “adenylate kinase”, preferably it is the molecule of section (a) or (b) of this paragraph.

In particular, it was observed that in plants, especially in Arabidopsis thaliana, increasing or generating the activity of a gene product mitochondrial with the activity of a “adenylate kinase”, preferably being encoded by a gene comprising the nucleic acid sequence SEQ ID NO. 85808, preferably the coding region thereof, conferred the production of or the increase in glycine compared with the wild type control.

The nucleic acid sequence of Sll1815 from Synechocystis sp., e.g. as shown in the respective line in column 5 of Table I, application no. 3, has been published in C. And the activity of the gene product thereof is the activity of adenylate kinase.

Accordingly, in one embodiment, the process of the present invention for producing serine in a non-human organism, like a microorganism or a plant or a part thereof, comprises increasing or generating the activity of a gene product with the activity of a gene product conferring the activity of “adenylate kinase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1815, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1815, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1815, or a functional equivalent or a homolog thereof as depicted in column 8 of Table II, application no. 3, preferably a homolog or functional equivalent as depicted in column 8 of Table II B, application no. 3, and being depicted in the same respective line as said Sll1815, and preferably the activity is increased mitochondrial.

Accordingly, in one embodiment, the molecule which activity is to be increased in the process of the invention is the gene product with an activity as a “adenylate kinase”, preferably it is the molecule of section (a) or (b) of this paragraph.

In particular, it was observed that in plants, especially in Arabidopsis thaliana, increasing or generating the activity of a gene product mitochondrial with the activity of a “adenylate kinase”, preferably being encoded by a gene comprising the nucleic acid sequence SEQ ID NO. 85808,preferably the coding region thereof, conferred the production of or the increase in serine compared with the wild type control.

The nucleic acid sequence of Sll1815 from Synechocystis sp., e.g. as shown in the respective line in column 5 of Table I, application no. 3, has been published in C. And the activity of the gene product thereof is the activity of adenylate kinase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “adenylate kinase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1815, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1815, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1815, or a functional equivalent or a homolog thereof as depicted in column 8 of Table II, application no. 3, preferably a homolog or functional equivalent as depicted in column 8 of Table II B, application no. 3, and being depicted in the same respective line as said Sll1815, and preferably the activity is increased mitochondrial.

Accordingly, in one embodiment, the molecule which activity is to be increased in the process of the invention is the gene product with an activity as a “adenylate kinase”, preferably it is the molecule of section (a) or (b) of this paragraph.

In particular, it was observed that in plants, especially in Arabidopsis thaliana, increasing or generating the activity of a gene product mitochondrial with the activity of a “adenylate kinase”, preferably being encoded by a gene comprising the nucleic acid sequence SEQ ID NO. 85808, preferably the coding region thereof, conferred the production of or the increase in tyrosine compared with the wild type control.

The nucleic acid sequence of Sll1848 from Synechocystis sp., e.g. as shown in the respective line in column 5 of Table I, application no. 3, has been published in C. And the activity of the gene product thereof is the activity of acyl transferase.

Accordingly, in one embodiment, the process of the present invention for producing glycine in a non-human organism, like a microorganism or a plant or a part thereof, comprises increasing or generating the activity of a gene product with the activity of a gene product conferring the activity of “acyl transferase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1848, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1848, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1848, or a functional equivalent or a homolog thereof as depicted in column 8 of Table II, application no. 3, preferably a homolog or functional equivalent as depicted in column 8 of Table II B, application no. 3, and being depicted in the same respective line as said Sll1848, and preferably the activity is increased plastidic.

Accordingly, in one embodiment, the molecule which activity is to be increased in the process of the invention is the gene product with an activity as a “acyl transferase”, preferably it is the molecule of section (a) or (b) of this paragraph.

In particular, it was observed that in plants, especially in Arabidopsis thaliana, increasing or generating the activity of a gene product plastidic with the activity of a “acyl transferase”, preferably being encoded by a gene comprising the nucleic acid sequence SEQ ID NO. 86447, preferably the coding region thereof, conferred the production of or the increase in glycine compared with the wild type control.

The nucleic acid sequence of Sll1848 from Synechocystis sp., e.g. as shown in the respective line in column 5 of Table I, application no. 3, has been published in C. And the activity of the gene product thereof is the activity of acyl transferase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “acyl transferase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1848, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1848, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1848, or a functional equivalent or a homolog thereof as depicted in column 8 of Table II, application no. 3, preferably a homolog or functional equivalent as depicted in column 8 of Table II B, application no. 3, and being depicted in the same respective line as said Sll1848, and preferably the activity is increased plastidic.

Accordingly, in one embodiment, the molecule which activity is to be increased in the process of the invention is the gene product with an activity as a “acyl transferase”, preferably it is the molecule of section (a) or (b) of this paragraph.

In particular, it was observed that in plants, especially in Arabidopsis thaliana, increasing or generating the activity of a gene product plastidic with the activity of a “acyl transferase”, preferably being encoded by a gene comprising the nucleic acid sequence SEQ ID NO. 86447, preferably the coding region thereof, conferred the production of or the increase in phenylalanine compared with the wild type control.

The nucleic acid sequence of Sll1848 from Synechocystis sp., e.g. as shown in the respective line in column 5 of Table I, application no. 3, has been published in C. And the activity of the gene product thereof is the activity of acyl transferase.

Accordingly, in one embodiment, the process of the present invention for producing serine in a non-human organism, like a microorganism or a plant or a part thereof, comprises increasing or generating the activity of a gene product with the activity of a gene product conferring the activity of “acyl transferase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1848, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1848, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1848, or a functional equivalent or a homolog thereof as depicted in column 8 of Table II, application no. 3, preferably a homolog or functional equivalent as depicted in column 8 of Table II B, application no. 3, and being depicted in the same respective line as said Sll1848, and preferably the activity is increased plastidic.

Accordingly, in one embodiment, the molecule which activity is to be increased in the process of the invention is the gene product with an activity as a “acyl transferase”, preferably it is the molecule of section (a) or (b) of this paragraph.

In particular, it was observed that in plants, especially in Arabidopsis thaliana, increasing or generating the activity of a gene product plastidic with the activity of a “acyl transferase”, preferably being encoded by a gene comprising the nucleic acid sequence SEQ ID NO. 86447, preferably the coding region thereof, conferred the production of or the increase in serine compared with the wild type control.

The nucleic acid sequence of Sll1917 from Synechocystis sp., e.g. as shown in the respective line in column 5 of Table I, application no. 3, has been published in C. And the activity of the gene product thereof is the activity of coproporphyrinogen III oxidase.

Accordingly, in one embodiment, the process of the present invention for producing aspartate in a non-human organism, like a microorganism or a plant or a part thereof, comprises increasing or generating the activity of a gene product with the activity of a gene product conferring the activity of “coproporphyrinogen III oxidase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1917, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1917, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1917, or a functional equivalent or a homolog thereof as depicted in column 8 of Table II, application no. 3, preferably a homolog or functional equivalent as depicted in column 8 of Table II B, application no. 3, and being depicted in the same respective line as said Sll1917, and preferably the activity is increased mitochondrial.

Accordingly, in one embodiment, the molecule which activity is to be increased in the process of the invention is the gene product with an activity as a “coproporphyrinogen III oxidase”, preferably it is the molecule of section (a) or (b) of this paragraph.

In particular, it was observed that in plants, especially in Arabidopsis thaliana, increasing or generating the activity of a gene product mitochondrial with the activity of a “coproporphyrinogen III oxidase”, preferably being encoded by a gene comprising the nucleic acid sequence SEQ ID NO. 11471, preferably the coding region thereof, conferred the production of or the increase in aspartate compared with the wild type control.

The nucleic acid sequence of Sll1917 from Synechocystis sp., e.g. as shown in the respective line in column 5 of Table I, application no. 3, has been published in C. And the activity of the gene product thereof is the activity of coproporphyrinogen III oxidase.

Accordingly, in one embodiment, the process of the present invention for producing citrulline in a non-human organism, like a microorganism or a plant or a part thereof, comprises increasing or generating the activity of a gene product with the activity of a gene product conferring the activity of “coproporphyrinogen III oxidase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1917, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1917, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1917, or a functional equivalent or a homolog thereof as depicted in column 8 of Table II, application no. 3, preferably a homolog or functional equivalent as depicted in column 8 of Table II B, application no. 3, and being depicted in the same respective line as said Sll1917, and preferably the activity is increased mitochondrial.

Accordingly, in one embodiment, the molecule which activity is to be increased in the process of the invention is the gene product with an activity as a “coproporphyrinogen III oxidase”, preferably it is the molecule of section (a) or (b) of this paragraph.

In particular, it was observed that in plants, especially in Arabidopsis thaliana, increasing or generating the activity of a gene product mitochondrial with the activity of a “coproporphyrinogen III oxidase”, preferably being encoded by a gene comprising the nucleic acid sequence SEQ ID NO. 11471, preferably the coding region thereof, conferred the production of or the increase in citrulline compared with the wild type control.

The nucleic acid sequence of Sll1917 from Synechocystis sp., e.g. as shown in the respective line in column 5 of Table I, application no. 3, has been published in C. And the activity of the gene product thereof is the activity of coproporphyrinogen III oxidase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “coproporphyrinogen III oxidase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1917, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1917, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1917, or a functional equivalent or a homolog thereof as depicted in column 8 of Table II, application no. 3, preferably a homolog or functional equivalent as depicted in column 8 of Table II B, application no. 3, and being depicted in the same respective line as said Sll1917, and preferably the activity is increased mitochondrial.

Accordingly, in one embodiment, the molecule which activity is to be increased in the process of the invention is the gene product with an activity as a “coproporphyrinogen III oxidase”, preferably it is the molecule of section (a) or (b) of this paragraph.

In particular, it was observed that in plants, especially in Arabidopsis thaliana, increasing or generating the activity of a gene product mitochondrial with the activity of a “coproporphyrinogen III oxidase”, preferably being encoded by a gene comprising the nucleic acid sequence SEQ ID NO. 11471, preferably the coding region thereof, conferred the production of or the increase in phenylalanine compared with the wild type control.

The nucleic acid sequence of Sll1917 from Synechocystis sp., e.g. as shown in the respective line in column 5 of Table I, application no. 3, has been published in C. And the activity of the gene product thereof is the activity of coproporphyrinogen III oxidase.

Accordingly, in one embodiment, the process of the present invention for producing serine in a non-human organism, like a microorganism or a plant or a part thereof, comprises increasing or generating the activity of a gene product with the activity of a gene product conferring the activity of “coproporphyrinogen Ill oxidase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1917, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1917, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1917, or a functional equivalent or a homolog thereof as depicted in column 8 of Table II, application no. 3, preferably a homolog or functional equivalent as depicted in column 8 of Table II B, application no. 3, and being depicted in the same respective line as said Sll1917, and preferably the activity is increased mitochondrial.

Accordingly, in one embodiment, the molecule which activity is to be increased in the process of the invention is the gene product with an activity as a “coproporphyrinogen III oxidase”, preferably it is the molecule of section (a) or (b) of this paragraph.

In particular, it was observed that in plants, especially in Arabidopsis thaliana, increasing or generating the activity of a gene product mitochondrial with the activity of a “coproporphyrinogen III oxidase”, preferably being encoded by a gene comprising the nucleic acid sequence SEQ ID NO. 11471, preferably the coding region thereof, conferred the production of or the increase in serine compared with the wild type control.

The nucleic acid sequence of Sll1917 from Synechocystis sp., e.g. as shown in the respective line in column 5 of Table I, application no. 3, has been published in C. And the activity of the gene product thereof is the activity of coproporphyrinogen III oxidase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “coproporphyrinogen III oxidase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1917, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1917, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1917, or a functional equivalent or a homolog thereof as depicted in column 8 of Table II, application no. 3, preferably a homolog or functional equivalent as depicted in column 8 of Table II B, application no. 3, and being depicted in the same respective line as said Sll1917, and preferably the activity is increased mitochondrial.

Accordingly, in one embodiment, the molecule which activity is to be increased in the process of the invention is the gene product with an activity as a “coproporphyrinogen III oxidase”, preferably it is the molecule of section (a) or (b) of this paragraph.

In particular, it was observed that in plants, especially in Arabidopsis thaliana, increasing or generating the activity of a gene product mitochondrial with the activity of a “coproporphyrinogen III oxidase”, preferably being encoded by a gene comprising the nucleic acid sequence SEQ ID NO. 11471, preferably the coding region thereof, conferred the production of or the increase in tyrosine compared with the wild type control.

The nucleic acid sequence of Sll1920 from Synechocystis sp., e.g. as shown in the respective line in column 5 of Table I, application no. 3, has been published in C. And the activity of the gene product thereof is the activity of cation-transporting ATPase.

Accordingly, in one embodiment, the process of the present invention for producing aspartate in a non-human organism, like a microorganism or a plant or a part thereof, comprises increasing or generating the activity of a gene product with the activity of a gene product conferring the activity of “cation-transporting ATPase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1920, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1920, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1920, or a functional equivalent or a homolog thereof as depicted in column 8 of Table II, application no. 3, preferably a homolog or functional equivalent as depicted in column 8 of Table II B, application no. 3, and being depicted in the same respective line as said Sll1920, and preferably the activity is increased non-targeted.

Accordingly, in one embodiment, the molecule which activity is to be increased in the process of the invention is the gene product with an activity as a “cation-transporting ATPase”, preferably it is the molecule of section (a) or (b) of this paragraph.

In particular, it was observed that in plants, especially in Arabidopsis thaliana, increasing or generating the activity of a gene product non-targeted with the activity of a “cation-transporting ATPase”, preferably being encoded by a gene comprising the nucleic acid sequence SEQ ID NO. 55385, preferably the coding region thereof, conferred the production of or the increase in aspartate compared with the wild type control.

The nucleic acid sequence of Sll1920 from Synechocystis sp., e.g. as shown in the respective line in column 5 of Table I, application no. 3, has been published in C. And the activity of the gene product thereof is the activity of cation-transporting ATPase.

Accordingly, in one embodiment, the process of the present invention for producing aspartate in a non-human organism, like a microorganism or a plant or a part thereof, comprises increasing or generating the activity of a gene product with the activity of a gene product conferring the activity of “cation-transporting ATPase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll1920, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll1920, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll1920, or a functional equivalent or a homolog thereof as depicted in column 8 of Table II, application no. 3, preferably a homolog or functional equivalent as depicted in column 8 of Table II B, application no. 3, and being depicted in the same respective line as said Sll1920, and preferably the activity is increased mitochondrial.

Accordingly, in one embodiment, the molecule which activity is to be increased in the process of the invention is the gene product with an activity as a “cation-transporting ATPase”, preferably it is the molecule of section (a) or (b) of this paragraph.

In particular, it was observed that in plants, especially in Arabidopsis thaliana, increasing or generating the activity of a gene product mitochondrial with the activity of a “cation-transporting ATPase”, preferably being encoded by a gene comprising the nucleic acid sequence SEQ ID NO. 55385, preferably the coding region thereof, conferred the production of or the increase in aspartate compared with the wild type control.

The nucleic acid sequence of Sll2003 from Synechocystis sp., e.g. as shown in the respective line in column 5 of Table I, application no. 3, has been published in C. And the activity of the gene product thereof is the activity of glycosyl transferase.

Accordingly, in one embodiment, the process of the present invention for producing glycine in a non-human organism, like a microorganism or a plant or a part thereof, comprises increasing or generating the activity of a gene product with the activity of a gene product conferring the activity of “glycosyl transferase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Sll2003, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Sll2003, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Sll2003, or a functional equivalent or a homolog thereof as depicted in column 8 of Table II, application no. 3, preferably a homolog or functional equivalent as depicted in column 8 of Table II B, application no. 3, and being depicted in the same respective line as said Sll2003, and preferably the activity is increased plastidic.

Accordingly, in one embodiment, the molecule which activity is to be increased in the process of the invention is the gene product with an activity as a “glycosyl transferase”, preferably it is the molecule of section (a) or (b) of this paragraph.

In particular, it was observed that in plants, especially in Arabidopsis thaliana, increasing or generating the activity of a gene product plastidic with the activity of a “glycosyl transferase”, preferably being encoded by a gene comprising the nucleic acid sequence SEQ ID NO. 86508, preferably the coding region thereof, conferred the production of or the increase in glycine compared with the wild type control.

The nucleic acid sequence of Slr0239 from Synechocystis sp., e.g. as shown in the respective line in column 5 of Table I, application no. 3, has been published in C. And the activity of the gene product thereof is the activity of precorrin methylase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “precorrin methylase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr0239, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr0239, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr0239, or a functional equivalent or a homolog thereof as depicted in column 8 of Table II, application no. 3, preferably a homolog or functional equivalent as depicted in column 8 of Table II B, application no. 3, and being depicted in the same respective line as said Slr0239, and preferably the activity is increased mitochondrial.

Accordingly, in one embodiment, the molecule which activity is to be increased in the process of the invention is the gene product with an activity as a “precorrin methylase”, preferably it is the molecule of section (a) or (b) of this paragraph.

In particular, it was observed that in plants, especially in Arabidopsis thaliana, increasing or generating the activity of a gene product mitochondrial with the activity of a “precorrin methylase”, preferably being encoded by a gene comprising the nucleic acid sequence SEQ ID NO. 86540, preferably the coding region thereof, conferred the production of or the increase in phenylalanine compared with the wild type control.

The nucleic acid sequence of Slr0239 from Synechocystis sp., e.g. as shown in the respective line in column 5 of Table I, application no. 3, has been published in C. And the activity of the gene product thereof is the activity of precorrin methylase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “precorrin methylase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr0239, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr0239, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr0239, or a functional equivalent or a homolog thereof as depicted in column 8 of Table II, application no. 3, preferably a homolog or functional equivalent as depicted in column 8 of Table II B, application no. 3, and being depicted in the same respective line as said Slr0239, and preferably the activity is increased mitochondrial.

Accordingly, in one embodiment, the molecule which activity is to be increased in the process of the invention is the gene product with an activity as a “precorrin methylase”, preferably it is the molecule of section (a) or (b) of this paragraph.

In particular, it was observed that in plants, especially in Arabidopsis thaliana, increasing or generating the activity of a gene product mitochondrial with the activity of a “precorrin methylase”, preferably being encoded by a gene comprising the nucleic acid sequence SEQ ID NO. 86540, preferably the coding region thereof, conferred the production of or the increase in tyrosine compared with the wild type control.

The nucleic acid sequence of Slr0370 from Synechocystis sp., e.g. as shown in the respective line in column 5 of Table I, application no. 3, has been published in C. And the activity of the gene product thereof is the activity of aldehyde dehydrogenase.

Accordingly, in one embodiment, the process of the present invention for producing citrulline in a non-human organism, like a microorganism or a plant or a part thereof, comprises increasing or generating the activity of a gene product with the activity of a gene product conferring the activity of “aldehyde dehydrogenase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr0370, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr0370, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr0370, or a functional equivalent or a homolog thereof as depicted in column 8 of Table II, application no. 3, preferably a homolog or functional equivalent as depicted in column 8 of Table II B, application no. 3, and being depicted in the same respective line as said Slr0370, and preferably the activity is increased plastidic.

Accordingly, in one embodiment, the molecule which activity is to be increased in the process of the invention is the gene product with an activity as a “aldehyde dehydrogenase”, preferably it is the molecule of section (a) or (b) of this paragraph.

In particular, it was observed that in plants, especially in Arabidopsis thaliana, increasing or generating the activity of a gene product plastidic with the activity of a “aldehyde dehydrogenase”, preferably being encoded by a gene comprising the nucleic acid sequence SEQ ID NO. 86820, preferably the coding region thereof, conferred the production of or the increase in citrulline compared with the wild type control.

The nucleic acid sequence of Slr0597 from Synechocystis sp., e.g. as shown in the respective line in column 5 of Table I, application no. 3, has been published in C. And the activity of the gene product thereof is the activity of bifunctional purine biosynthesis protein.

Accordingly, in one embodiment, the process of the present invention for producing alanine in a non-human organism, like a microorganism or a plant or a part thereof, comprises increasing or generating the activity of a gene product with the activity of a gene product conferring the activity of “bifunctional purine biosynthesis protein”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr0597, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr0597, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr0597, or a functional equivalent or a homolog thereof as depicted in column 8 of Table

II, application no. 3, preferably a homolog or functional equivalent as depicted in column 8 of Table II B, application no. 3, and being depicted in the same respective line as said Slr0597, and preferably the activity is increased plastidic.

Accordingly, in one embodiment, the molecule which activity is to be increased in the process of the invention is the gene product with an activity as a “bifunctional purine biosynthesis protein”, preferably it is the molecule of section (a) or (b) of this paragraph.

In particular, it was observed that in plants, especially in Arabidopsis thaliana, increasing or generating the activity of a gene product plastidic with the activity of a “bifunctional purine biosynthesis protein”, preferably being encoded by a gene comprising the nucleic acid sequence SEQ ID NO. 56153, preferably the coding region thereof, conferred the production of or the increase in alanine compared with the wild type control.

The nucleic acid sequence of Slr0597 from Synechocystis sp., e.g. as shown in the respective line in column 5 of Table I, application no. 3, has been published in C. And the activity of the gene product thereof is the activity of bifunctional purine biosynthesis protein.

Accordingly, in one embodiment, the process of the present invention for producing serine in a non-human organism, like a microorganism or a plant or a part thereof, comprises increasing or generating the activity of a gene product with the activity of a gene product conferring the activity of “bifunctional purine biosynthesis protein”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr0597, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr0597, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr0597, or a functional equivalent or a homolog thereof as depicted in column 8 of Table II, application no. 3, preferably a homolog or functional equivalent as depicted in column 8 of Table II B, application no. 3, and being depicted in the same respective line as said Slr0597, and preferably the activity is increased plastidic.

Accordingly, in one embodiment, the molecule which activity is to be increased in the process of the invention is the gene product with an activity as a “bifunctional purine biosynthesis protein”, preferably it is the molecule of section (a) or (b) of this paragraph.

In particular, it was observed that in plants, especially in Arabidopsis thaliana, increasing or generating the activity of a gene product plastidic with the activity of a “bifunctional purine biosynthesis protein”, preferably being encoded by a gene comprising the nucleic acid sequence SEQ ID NO. 56153, preferably the coding region thereof, conferred the production of or the increase in serine compared with the wild type control.

The nucleic acid sequence of Slr0597 from Synechocystis sp., e.g. as shown in the respective line in column 5 of Table I, application no. 3, has been published in C. And the activity of the gene product thereof is the activity of bifunctional purine biosynthesis protein.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “bifunctional purine biosynthesis protein”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr0597, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr0597, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr0597, or a functional equivalent or a homolog thereof as depicted in column 8 of Table II, application no. 3, preferably a homolog or functional equivalent as depicted in column 8 of Table II B, application no. 3, and being depicted in the same respective line as said Slr0597, and preferably the activity is increased plastidic.

Accordingly, in one embodiment, the molecule which activity is to be increased in the process of the invention is the gene product with an activity as a “bifunctional purine biosynthesis protein”, preferably it is the molecule of section (a) or (b) of this paragraph.

In particular, it was observed that in plants, especially in Arabidopsis thaliana, increasing or generating the activity of a gene product plastidic with the activity of a “bifunctional purine biosynthesis protein”, preferably being encoded by a gene comprising the nucleic acid sequence SEQ ID NO. 56153, preferably the coding region thereof, conferred the production of or the increase in 5-oxoproline compared with the wild type control.

The nucleic acid sequence of Slr0710 from Synechocystis sp., e.g. as shown in the respective line in column 5 of Table I, application no. 3, has been published in C. And the activity of the gene product thereof is the activity of Glu/Leu/Phe/Val dehydrogenase.

Accordingly, in one embodiment, the process of the present invention for producing asparagine in a non-human organism, like a microorganism or a plant or a part thereof, comprises increasing or generating the activity of a gene product with the activity of a gene product conferring the activity of “Glu/Leu/Phe/Val dehydrogenase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr0710, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr0710, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr0710, or a functional equivalent or a homolog thereof as depicted in column 8 of Table II, application no. 3, preferably a homolog or functional equivalent as depicted in column 8 of Table II B, application no. 3, and being depicted in the same respective line as said Slr0710, and preferably the activity is increased mitochondrial.

Accordingly, in one embodiment, the molecule which activity is to be increased in the process of the invention is the gene product with an activity as a “Glu/Leu/Phe/Val dehydrogenase”, preferably it is the molecule of section (a) or (b) of this paragraph.

In particular, it was observed that in plants, especially in Arabidopsis thaliana, increasing or generating the activity of a gene product mitochondrial with the activity of a “Glu/Leu/Phe/Val dehydrogenase”, preferably being encoded by a gene comprising the nucleic acid sequence SEQ ID NO. 56894, preferably the coding region thereof, conferred the production of or the increase in asparagine compared with the wild type control.

The nucleic acid sequence of Slr0710 from Synechocystis sp., e.g. as shown in the respective line in column 5 of Table I, application no. 3, has been published in C. And the activity of the gene product thereof is the activity of Glu/Leu/Phe/Val dehydrogenase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “Glu/Leu/Phe/Val dehydrogenase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr0710, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr0710, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr0710, or a functional equivalent or a homolog thereof as depicted in column 8 of Table II, application no. 3, preferably a homolog or functional equivalent as depicted in column 8 of Table II B, application no. 3, and being depicted in the same respective line as said Slr0710, and preferably the activity is increased mitochondrial.

Accordingly, in one embodiment, the molecule which activity is to be increased in the process of the invention is the gene product with an activity as a “Glu/Leu/Phe/Val dehydrogenase”, preferably it is the molecule of section (a) or (b) of this paragraph.

In particular, it was observed that in plants, especially in Arabidopsis thaliana, increasing or generating the activity of a gene product mitochondrial with the activity of a “Glu/Leu/Phe/Val dehydrogenase”, preferably being encoded by a gene comprising the nucleic acid sequence SEQ ID NO. 56894, preferably the coding region thereof, conferred the production of or the increase in phenylalanine compared with the wild type control.

The nucleic acid sequence of Slr0710 from Synechocystis sp., e.g. as shown in the respective line in column 5 of Table I, application no. 3, has been published in C. And the activity of the gene product thereof is the activity of Glu/Leu/Phe/Val dehydrogenase .

Accordingly, in one embodiment, the process of the present invention for producing tyrosine in a non-human organism, like a microorganism or a plant or a part thereof, comprises increasing or generating the activity of a gene product with the activity of a gene product conferring the activity of “Glu/Leu/PheNal dehydrogenase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr0710, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr0710, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr0710, or a functional equivalent or a homolog thereof as depicted in column 8 of Table II, application no. 3, preferably a homolog or functional equivalent as depicted in column 8 of Table II B, application no. 3, and being depicted in the same respective line as said Slr0710, and preferably the activity is increased mitochondrial.

Accordingly, in one embodiment, the molecule which activity is to be increased in the process of the invention is the gene product with an activity as a “Glu/Leu/Phe/Val dehydrogenase”, preferably it is the molecule of section (a) or (b) of this paragraph.

In particular, it was observed that in plants, especially in Arabidopsis thaliana, increasing or generating the activity of a gene product mitochondrial with the activity of a “Glu/Leu/PheNal dehydrogenase”, preferably being encoded by a gene comprising the nucleic acid sequence SEQ ID NO. 56894, preferably the coding region thereof, conferred the production of or the increase in tyrosine compared with the wild type control.

The nucleic acid sequence of Slr0721 from Synechocystis sp., e.g. as shown in the respective line in column 5 of Table I, application no. 3, has been published in C. And the activity of the gene product thereof is the activity of malate dehydrogenase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “malate dehydrogenase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr0721, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr0721, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr0721, or a functional equivalent or a homolog thereof as depicted in column 8 of Table II, application no. 3, preferably a homolog or functional equivalent as depicted in column 8 of Table II B, application no. 3, and being depicted in the same respective line as said Slr0721, and preferably the activity is increased plastidic.

Accordingly, in one embodiment, the molecule which activity is to be increased in the process of the invention is the gene product with an activity as a “malate dehydrogenase”, preferably it is the molecule of section (a) or (b) of this paragraph.

In particular, it was observed that in plants, especially in Arabidopsis thaliana, increasing or generating the activity of a gene product plastidic with the activity of a “malate dehydrogenase”, preferably being encoded by a gene comprising the nucleic acid sequence SEQ ID NO. 87397, preferably the coding region thereof, conferred the production of or the increase in phenylalanine compared with the wild type control.

The nucleic acid sequence of Slr0739 from Synechocystis sp., e.g. as shown in the respective line in column 5 of Table I, application no. 3, has been published in C. And the activity of the gene product thereof is the activity of geranylgeranyl pyrophosphate synthase.

Accordingly, in one embodiment, the process of the present invention for producing alanine in a non-human organism, like a microorganism or a plant or a part thereof, comprises increasing or generating the activity of a gene product with the activity of a gene product conferring the activity of “geranylgeranyl pyrophosphate synthase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr0739, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr0739, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr0739, or a functional equivalent or a homolog thereof as depicted in column 8 of Table II, application no. 3, preferably a homolog or functional equivalent as depicted in column 8 of Table II B, application no. 3, and being depicted in the same respective line as said Slr0739, and preferably the activity is increased plastidic.

Accordingly, in one embodiment, the molecule which activity is to be increased in the process of the invention is the gene product with an activity as a “geranylgeranyl pyrophosphate synthase”, preferably it is the molecule of section (a) or (b) of this paragraph.

In particular, it was observed that in plants, especially in Arabidopsis thaliana, increasing or generating the activity of a gene product plastidic with the activity of a “geranylgeranyl pyrophosphate synthase”, preferably being encoded by a gene comprising the nucleic acid sequence SEQ ID NO. 57235, preferably the coding region thereof, conferred the production of or the increase in alanine compared with the wild type control.

The nucleic acid sequence of Slr0739 from Synechocystis sp., e.g. as shown in the respective line in column 5 of Table I, application no. 3, has been published in C. And the activity of the gene product thereof is the activity of geranylgeranyl pyrophosphate synthase.

Accordingly, in one embodiment, the process of the present invention for producing asparagine in a non-human organism, like a microorganism or a plant or a part thereof, comprises increasing or generating the activity of a gene product with the activity of a gene product conferring the activity of “geranylgeranyl pyrophosphate synthase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr0739, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr0739, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr0739, or a functional equivalent or a homolog thereof as depicted in column 8 of Table II, application no. 3, preferably a homolog or functional equivalent as depicted in column 8 of Table II B, application no. 3, and being depicted in the same respective line as said Slr0739, and preferably the activity is increased plastidic.

Accordingly, in one embodiment, the molecule which activity is to be increased in the process of the invention is the gene product with an activity as a “geranylgeranyl pyrophosphate synthase”, preferably it is the molecule of section (a) or (b) of this paragraph.

In particular, it was observed that in plants, especially in Arabidopsis thaliana, increasing or generating the activity of a gene product plastidic with the activity of a “geranylgeranyl pyrophosphate synthase”, preferably being encoded by a gene comprising the nucleic acid sequence SEQ ID NO. 57235, preferably the coding region thereof, conferred the production of or the increase in asparagine compared with the wild type control.

The nucleic acid sequence of Slr0739 from Synechocystis sp., e.g. as shown in the respective line in column 5 of Table I, application no. 3, has been published in C. And the activity of the gene product thereof is the activity of geranylgeranyl pyrophosphate synthase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “geranylgeranyl pyrophosphate synthase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr0739, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr0739, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr0739, or a functional equivalent or a homolog thereof as depicted in column 8 of Table II, application no. 3, preferably a homolog or functional equivalent as depicted in column 8 of Table II B, application no. 3, and being depicted in the same respective line as said Slr0739, and preferably the activity is increased plastidic.

Accordingly, in one embodiment, the molecule which activity is to be increased in the process of the invention is the gene product with an activity as a “geranylgeranyl pyrophosphate synthase”, preferably it is the molecule of section (a) or (b) of this paragraph.

In particular, it was observed that in plants, especially in Arabidopsis thaliana, increasing or generating the activity of a gene product plastidic with the activity of a “geranylgeranyl pyrophosphate synthase”, preferably being encoded by a gene comprising the nucleic acid sequence SEQ ID NO. 57235, preferably the coding region thereof, conferred the production of or the increase in phenylalanine compared with the wild type control.

The nucleic acid sequence of Slr0739 from Synechocystis sp., e.g. as shown in the respective line in column 5 of Table I, application no. 3, has been published in C. And the activity of the gene product thereof is the activity of geranylgeranyl pyrophosphate synthase.

Accordingly, in one embodiment, the process of the present invention for producing serine in a non-human organism, like a microorganism or a plant or a part thereof, comprises increasing or generating the activity of a gene product with the activity of a gene product conferring the activity of “geranylgeranyl pyrophosphate synthase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr0739, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr0739, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr0739, or a functional equivalent or a homolog thereof as depicted in column 8 of Table II, application no. 3, preferably a homolog or functional equivalent as depicted in column 8 of Table II B, application no. 3, and being depicted in the same respective line as said Slr0739, and preferably the activity is increased plastidic.

Accordingly, in one embodiment, the molecule which activity is to be increased in the process of the invention is the gene product with an activity as a “geranylgeranyl pyrophosphate synthase”, preferably it is the molecule of section (a) or (b) of this paragraph.

In particular, it was observed that in plants, especially in Arabidopsis thaliana, increasing or generating the activity of a gene product plastidic with the activity of a “geranylgeranyl pyrophosphate synthase”, preferably being encoded by a gene comprising the nucleic acid sequence SEQ ID NO. 57235, preferably the coding region thereof, conferred the production of or the increase in serine compared with the wild type control.

The nucleic acid sequence of Slr0739 from Synechocystis sp., e.g. as shown in the respective line in column 5 of Table I, application no. 3, has been published in C. And the activity of the gene product thereof is the activity of geranylgeranyl pyrophosphate synthase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “geranylgeranyl pyrophosphate synthase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr0739, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr0739, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr0739, or a functional equivalent or a homolog thereof as depicted in column 8 of Table II, application no. 3, preferably a homolog or functional equivalent as depicted in column 8 of Table II B, application no. 3, and being depicted in the same respective line as said Slr0739, and preferably the activity is increased plastidic.

Accordingly, in one embodiment, the molecule which activity is to be increased in the process of the invention is the gene product with an activity as a “geranylgeranyl pyrophosphate synthase”, preferably it is the molecule of section (a) or (b) of this paragraph.

In particular, it was observed that in plants, especially in Arabidopsis thaliana, increasing or generating the activity of a gene product plastidic with the activity of a “geranylgeranyl pyrophosphate synthase”, preferably being encoded by a gene comprising the nucleic acid sequence SEQ ID NO. 57235, preferably the coding region thereof, conferred the production of or the increase in tyrosine compared with the wild type control.

The nucleic acid sequence of Slr0739 from Synechocystis sp., e.g. as shown in the respective line in column 5 of Table I, application no. 3, has been published in C. And the activity of the gene product thereof is the activity of geranylgeranyl pyrophosphate synthase.

Accordingly, in one embodiment, the process of the present invention for producing 5oxoproline in a non-human organism, like a microorganism or a plant or a part thereof, comprises increasing or generating the activity of a gene product with the activity of a gene product conferring the activity of “geranylgeranyl pyrophosphate synthase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr0739, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr0739, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr0739, or a functional equivalent or a homolog thereof as depicted in column 8 of Table II, application no. 3, preferably a homolog or functional equivalent as depicted in column 8 of Table II B, application no. 3, and being depicted in the same respective line as said Slr0739, and preferably the activity is increased plastidic.

Accordingly, in one embodiment, the molecule which activity is to be increased in the process of the invention is the gene product with an activity as a “geranylgeranyl pyrophosphate synthase”, preferably it is the molecule of section (a) or (b) of this paragraph.

In particular, it was observed that in plants, especially in Arabidopsis thaliana, increasing or generating the activity of a gene product plastidic with the activity of a “geranylgeranyl pyrophosphate synthase”, preferably being encoded by a gene comprising the nucleic acid sequence SEQ ID NO. 57235, preferably the coding region thereof, conferred the production of or the increase in 5-oxoproline compared with the wild type control.

The nucleic acid sequence of Slr0782 from Synechocystis sp., e.g. as shown in the respective line in column 5 of Table I, application no. 3, has been published in C. And the activity of the gene product thereof is the activity of amine oxidase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “amine oxidase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr0782, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr0782, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr0782, or a functional equivalent or a homolog thereof as depicted in column 8 of Table II, application no. 3, preferably a homolog or functional equivalent as depicted in column 8 of Table II B, application no. 3, and being depicted in the same respective line as said Slr0782, and preferably the activity is increased mitochondrial.

Accordingly, in one embodiment, the molecule which activity is to be increased in the process of the invention is the gene product with an activity as a “amine oxidase”, preferably it is the molecule of section (a) or (b) of this paragraph.

In particular, it was observed that in plants, especially in Arabidopsis thaliana, increasing or generating the activity of a gene product mitochondrial with the activity of a “amine oxidase”, preferably being encoded by a gene comprising the nucleic acid sequence SEQ ID NO. 57679, preferably the coding region thereof, conferred the production of or the increase in phenylalanine compared with the wild type control.

The nucleic acid sequence of Slr0862 from Synechocystis sp., e.g. as shown in the respective line in column 5 of Table I, application no. 3, has been published in C. And the activity of the gene product thereof is the activity of kinase.

Accordingly, in one embodiment, the process of the present invention for producing serine in a non-human organism, like a microorganism or a plant or a part thereof, comprises increasing or generating the activity of a gene product with the activity of a gene product conferring the activity of “kinase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr0862, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr0862, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr0862, or a functional equivalent or a homolog thereof as depicted in column 8 of Table II, application no. 3, preferably a homolog or functional equivalent as depicted in column 8 of Table II B, application no. 3, and being depicted in the same respective line as said Slr0862, and preferably the activity is increased mitochondrial.

Accordingly, in one embodiment, the molecule which activity is to be increased in the process of the invention is the gene product with an activity as a “kinase”, preferably it is the molecule of section (a) or (b) of this paragraph.

In particular, it was observed that in plants, especially in Arabidopsis thaliana, increasing or generating the activity of a gene product mitochondrial with the activity of a “kinase”, preferably being encoded by a gene comprising the nucleic acid sequence SEQ ID NO. 87610, preferably the coding region thereof, conferred the production of or the increase in serine compared with the wild type control.

The nucleic acid sequence of Slr0966 from Synechocystis sp., e.g. as shown in the respective line in column 5 of Table I, application no. 3, has been published in C. And the activity of the gene product thereof is the activity of tryptophan synthase alpha chain.

Accordingly, in one embodiment, the process of the present invention for producing tyrosine in a non-human organism, like a microorganism or a plant or a part thereof, comprises increasing or generating the activity of a gene product with the activity of a gene product conferring the activity of “tryptophan synthase alpha chain”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr0966, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr0966, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr0966, or a functional equivalent or a homolog thereof as depicted in column 8 of Table II, application no. 3, preferably a homolog or functional equivalent as depicted in column 8 of Table II B, application no. 3, and being depicted in the same respective line as said Slr0966, and preferably the activity is increased plastidic.

Accordingly, in one embodiment, the molecule which activity is to be increased in the process of the invention is the gene product with an activity as a “tryptophan synthase alpha chain”, preferably it is the molecule of section (a) or (b) of this paragraph.

In particular, it was observed that in plants, especially in Arabidopsis thaliana, increasing or generating the activity of a gene product plastidic with the activity of a “tryptophan synthase alpha chain”, preferably being encoded by a gene comprising the nucleic acid sequence SEQ ID NO. 87655, preferably the coding region thereof, conferred the production of or the increase in tyrosine compared with the wild type control.

The nucleic acid sequence of Slr1133 from Synechocystis sp., e.g. as shown in the respective line in column 5 of Table I, application no. 3, has been published in C. And the activity of the gene product thereof is the activity of argininosuccinate lyase.

Accordingly, in one embodiment, the process of the present invention for producing ornithine in a non-human organism, like a microorganism or a plant or a part thereof, comprises increasing or generating the activity of a gene product with the activity of a gene product conferring the activity of “argininosuccinate lyase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr1133, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr1133, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr1133, or a functional equivalent or a homolog thereof as depicted in column 8 of Table II, application no. 3, preferably a homolog or functional equivalent as depicted in column 8 of Table II B, application no. 3, and being depicted in the same respective line as said Slr1133, and preferably the activity is increased mitochondrial.

Accordingly, in one embodiment, the molecule which activity is to be increased in the process of the invention is the gene product with an activity as a “argininosuccinate lyase”, preferably it is the molecule of section (a) or (b) of this paragraph.

In particular, it was observed that in plants, especially in Arabidopsis thaliana, increasing or generating the activity of a gene product mitochondrial with the activity of a “argininosuccinate lyase”, preferably being encoded by a gene comprising the nucleic acid sequence SEQ ID NO. 87853, preferably the coding region thereof, conferred the production of or the increase in ornithine compared with the wild type control.

The nucleic acid sequence of Slr1133 from Synechocystis sp., e.g. as shown in the respective line in column 5 of Table I, application no. 3, has been published in C. And the activity of the gene product thereof is the activity of argininosuccinate lyase.

Accordingly, in one embodiment, the process of the present invention for producing serine in a non-human organism, like a microorganism or a plant or a part thereof, comprises increasing or generating the activity of a gene product with the activity of a gene product conferring the activity of “argininosuccinate lyase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr1133, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr1133, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr1133, or a functional equivalent or a homolog thereof as depicted in column 8 of Table II, application no. 3, preferably a homolog or functional equivalent as depicted in column 8 of Table II B, application no. 3, and being depicted in the same respective line as said Slr1133, and preferably the activity is increased mitochondrial.

Accordingly, in one embodiment, the molecule which activity is to be increased in the process of the invention is the gene product with an activity as a “argininosuccinate lyase”, preferably it is the molecule of section (a) or (b) of this paragraph.

In particular, it was observed that in plants, especially in Arabidopsis thaliana, increasing or generating the activity of a gene product mitochondrial with the activity of a “argininosuccinate lyase”, preferably being encoded by a gene comprising the nucleic acid sequence SEQ ID NO. 87853, preferably the coding region thereof, conferred the production of or the increase in serine compared with the wild type control.

The nucleic acid sequence of Slr1269 from Synechocystis sp., e.g. as shown in the respective line in column 5 of Table I, application no. 3, has been published in C. And the activity of the gene product thereof is the activity of gamma-glutamyltranspeptidase.

Accordingly, in one embodiment, the process of the present invention for producing asparagine in a non-human organism, like a microorganism or a plant or a part thereof, comprises increasing or generating the activity of a gene product with the activity of a gene product conferring the activity of “gamma-glutamyltranspeptidase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr1269, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr1269, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr1269, or a functional equivalent or a homolog thereof as depicted in column 8 of Table II, application no. 3, preferably a homolog or functional equivalent as depicted in column 8 of Table II B, application no. 3, and being depicted in the same respective line as said Slr1269, and preferably the activity is increased non-targeted.

Accordingly, in one embodiment, the molecule which activity is to be increased in the process of the invention is the gene product with an activity as a “gamma-glutamyltranspeptidase”, preferably it is the molecule of section (a) or (b) of this paragraph.

In particular, it was observed that in plants, especially in Arabidopsis thaliana, increasing or generating the activity of a gene product non-targeted with the activity of a “gamma-glutamyltranspeptidase”, preferably being encoded by a gene comprising the nucleic acid sequence SEQ ID NO. 58058, preferably the coding region thereof, conferred the production of or the increase in asparagine compared with the wild type control.

The nucleic acid sequence of Slr1492 from Synechocystis sp., e.g. as shown in the respective line in column 5 of Table I, application no. 3, has been published in C. And the activity of the gene product thereof is the activity of iron(III) dicitrate-binding protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “iron(III) dicitrate-binding protein”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr1492, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr1492, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr1492, or a functional equivalent or a homolog thereof as depicted in column 8 of Table II, application no. 3, preferably a homolog or functional equivalent as depicted in column 8 of Table II B, application no. 3, and being depicted in the same respective line as said Slr1492, and preferably the activity is increased mitochondrial.

Accordingly, in one embodiment, the molecule which activity is to be increased in the process of the invention is the gene product with an activity as a “iron(III) dicitrate-binding protein”, preferably it is the molecule of section (a) or (b) of this paragraph.

In particular, it was observed that in plants, especially in Arabidopsis thaliana, increasing or generating the activity of a gene product mitochondrial with the activity of a “iron(III) dicitrate-binding protein”, preferably being encoded by a gene comprising the nucleic acid sequence SEQ ID NO. 58668, preferably the coding region thereof, conferred the production of or the increase in aspartate compared with the wild type control.

The nucleic acid sequence of Slr1492 from Synechocystis sp., e.g. as shown in the respective line in column 5 of Table I, application no. 3, has been published in C. And the activity of the gene product thereof is the activity of iron(III) dicitrate-binding protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “iron(III) dicitrate-binding protein”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr1492, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr1492, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr1492, or a functional equivalent or a homolog thereof as depicted in column 8 of Table II, application no. 3, preferably a homolog or functional equivalent as depicted in column 8 of Table II B, application no. 3, and being depicted in the same respective line as said Slr1492, and preferably the activity is increased mitochondrial.

Accordingly, in one embodiment, the molecule which activity is to be increased in the process of the invention is the gene product with an activity as a “iron(III) dicitrate-binding protein”, preferably it is the molecule of section (a) or (b) of this paragraph.

In particular, it was observed that in plants, especially in Arabidopsis thaliana, increasing or generating the activity of a gene product mitochondrial with the activity of a “iron(III) dicitratebinding protein”, preferably being encoded by a gene comprising the nucleic acid sequence SEQ ID NO. 58668, preferably the coding region thereof, conferred the production of or the increase in phenylalanine compared with the wild type control.

The nucleic acid sequence of Slr1509 from Synechocystis sp., e.g. as shown in the respective line in column 5 of Table I, application no. 3, has been published in C. And the activity of the gene product thereof is the activity of Na+/K+ transporter.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “Na+/K+ transporter”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr1509, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr1509, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr1509, or a functional equivalent or a homolog thereof as depicted in column 8 of Table II, application no. 3, preferably a homolog or functional equivalent as depicted in column 8 of Table II B, application no. 3, and being depicted in the same respective line as said Slr1509, and preferably the activity is increased mitochondrial.

Accordingly, in one embodiment, the molecule which activity is to be increased in the process of the invention is the gene product with an activity as a “Na+/K+ transporter”, preferably it is the molecule of section (a) or (b) of this paragraph.

In particular, it was observed that in plants, especially in Arabidopsis thaliana, increasing or generating the activity of a gene product mitochondrial with the activity of a “Na+/K+ transporter”, preferably being encoded by a gene comprising the nucleic acid sequence SEQ ID NO. 88158, preferably the coding region thereof, conferred the production of or the increase in phenylalanine compared with the wild type control.

The nucleic acid sequence of Slr1509 from Synechocystis sp., e.g. as shown in the respective line in column 5 of Table I, application no. 3, has been published in C. And the activity of the gene product thereof is the activity of Na+/K+ transporter.

Accordingly, in one embodiment, the process of the present invention for producing tyrosine in a non-human organism, like a microorganism or a plant or a part thereof, comprises increasing or generating the activity of a gene product with the activity of a gene product conferring the activity of “Na+/K+ transporter”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr1509, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr1509, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr1509, or a functional equivalent or a homolog thereof as depicted in column 8 of Table II, application no. 3, preferably a homolog or functional equivalent as depicted in column 8 of Table II B, application no. 3, and being depicted in the same respective line as said Slr1509, and preferably the activity is increased mitochondrial.

Accordingly, in one embodiment, the molecule which activity is to be increased in the process of the invention is the gene product with an activity as a “Na+/K+ transporter”, preferably it is the molecule of section (a) or (b) of this paragraph.

In particular, it was observed that in plants, especially in Arabidopsis thaliana, increasing or generating the activity of a gene product mitochondrial with the activity of a “Na+/K+ transporter”, preferably being encoded by a gene comprising the nucleic acid sequence SEQ ID NO. 88158, preferably the coding region thereof, conferred the production of or the increase in tyrosine compared with the wild type control.

The nucleic acid sequence of Slr1511 from Synechocystis sp., e.g. as shown in the respective line in column 5 of Table I, application no. 3, has been published in C. And the activity of the gene product thereof is the activity of 3-oxoacyl-[acyl-carrier-protein] synthase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “3-oxoacyl-[acyl-carrier-protein] synthase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr1511, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr1511, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr1511, or a functional equivalent or a homolog thereof as depicted in column 8 of Table II, application no. 3, preferably a homolog or functional equivalent as depicted in column 8 of Table II B, application no. 3, and being depicted in the same respective line as said Slr1511, and preferably the activity is increased non-targeted.

Accordingly, in one embodiment, the molecule which activity is to be increased in the process of the invention is the gene product with an activity as a “3-oxoacyl-[acyl-carrier-protein] synthase”, preferably it is the molecule of section (a) or (b) of this paragraph.

In particular, it was observed that in plants, especially in Arabidopsis thaliana, increasing or generating the activity of a gene product non-targeted with the activity of a “3-oxoacyl-[acyl-carrier-protein] synthase”, preferably being encoded by a gene comprising the nucleic acid sequence SEQ ID NO. 88271, preferably the coding region thereof, conferred the production of or the increase in phenylalanine compared with the wild type control.

The nucleic acid sequence of Slr1511 from Synechocystis sp., e.g. as shown in the respective line in column 5 of Table I, application no. 3, has been published in C. And the activity of the gene product thereof is the activity of 3-oxoacyl-[acyl-carrier-protein] synthase.

Accordingly, in one embodiment, the process of the present invention for producing serine in a non-human organism, like a microorganism or a plant or a part thereof, comprises increasing or generating the activity of a gene product with the activity of a gene product conferring the activity of “3-oxoacyl-[acyl-carrier-protein] synthase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr1511, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr1511, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr1511, or a functional equivalent or a homolog thereof as depicted in column 8 of Table II, application no. 3, preferably a homolog or functional equivalent as depicted in column 8 of Table II B, application no. 3, and being depicted in the same respective line as said Slr1511, and preferably the activity is increased non-targeted.

Accordingly, in one embodiment, the molecule which activity is to be increased in the process of the invention is the gene product with an activity as a “3-oxoacyl-[acyl-carrier-protein] synthase”, preferably it is the molecule of section (a) or (b) of this paragraph.

In particular, it was observed that in plants, especially in Arabidopsis thaliana, increasing or generating the activity of a gene product non-targeted with the activity of a “3-oxoacyl-[acyl-carrier-protein] synthase”, preferably being encoded by a gene comprising the nucleic acid sequence SEQ ID NO. 88271, preferably the coding region thereof, conferred the production of or the increase in serine compared with the wild type control.

The nucleic acid sequence of Slr1655 from Synechocystis sp., e.g. as shown in the respective line in column 5 of Table I, application no. 3, has been published in C. And the activity of the gene product thereof is the activity of Photosystem I reaction center subunit XI.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “Photosystem I reaction center subunit XI”, especially from Synechocystis sp.

or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr1655, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr1655, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr1655, or a functional equivalent or a homolog thereof as depicted in column 8 of Table II, application no. 3, preferably a homolog or functional equivalent as depicted in column 8 of Table II B, application no. 3, and being depicted in the same respective line as said Slr1655, and preferably the activity is increased mitochondrial.

Accordingly, in one embodiment, the molecule which activity is to be increased in the process of the invention is the gene product with an activity as a “Photosystem I reaction center subunit XI”, preferably it is the molecule of section (a) or (b) of this paragraph.

In particular, it was observed that in plants, especially in Arabidopsis thaliana, increasing or generating the activity of a gene product mitochondrial with the activity of a “Photosystem I reaction center subunit XI”, preferably being encoded by a gene comprising the nucleic acid sequence SEQ ID NO. 12070, preferably the coding region thereof, conferred the production of or the increase in phenylalanine compared with the wild type control.

The nucleic acid sequence of Slr1655 from Synechocystis sp., e.g. as shown in the respective line in column 5 of Table I, application no. 3, has been published in C. And the activity of the gene product thereof is the activity of Photosystem I reaction center subunit XI.

Accordingly, in one embodiment, the process of the present invention for producing serine in a non-human organism, like a microorganism or a plant or a part thereof, comprises increasing or generating the activity of a gene product with the activity of a gene product conferring the activity of “Photosystem I reaction center subunit XI”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr1655, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr1655, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr1655, or a functional equivalent or a homolog thereof as depicted in column 8 of Table II, application no. 3, preferably a homolog or functional equivalent as depicted in column 8 of Table II B, application no. 3, and being depicted in the same respective line as said Slr1655, and preferably the activity is increased mitochondrial.

Accordingly, in one embodiment, the molecule which activity is to be increased in the process of the invention is the gene product with an activity as a “Photosystem I reaction center subunit XI”, preferably it is the molecule of section (a) or (b) of this paragraph.

In particular, it was observed that in plants, especially in Arabidopsis thaliana, increasing or generating the activity of a gene product mitochondrial with the activity of a “Photosystem I reaction center subunit XI”, preferably being encoded by a gene comprising the nucleic acid sequence SEQ ID NO. 12070, preferably the coding region thereof, conferred the production of or the increase in serine compared with the wild type control.

The nucleic acid sequence of Slr1655 from Synechocystis sp., e.g. as shown in the respective line in column 5 of Table I, application no. 3, has been published in C. And the activity of the gene product thereof is the activity of Photosystem I reaction center subunit XI.

Accordingly, in one embodiment, the process of the present invention for producing tyrosine in a non-human organism, like a microorganism or a plant or a part thereof, comprises increasing or generating the activity of a gene product with the activity of a gene product conferring the activity of “Photosystem I reaction center subunit XI”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr1655, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr1655, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr1655, or a functional equivalent or a homolog thereof as depicted in column 8 of Table II, application no. 3, preferably a homolog or functional equivalent as depicted in column 8 of Table II B, application no. 3, and being depicted in the same respective line as said Slr1655, and preferably the activity is increased mitochondrial.

Accordingly, in one embodiment, the molecule which activity is to be increased in the process of the invention is the gene product with an activity as a “Photosystem I reaction center subunit

XI”, preferably it is the molecule of section (a) or (b) of this paragraph.

In particular, it was observed that in plants, especially in Arabidopsis thaliana, increasing or generating the activity of a gene product mitochondrial with the activity of a “Photosystem I reaction center subunit XI”, preferably being encoded by a gene comprising the nucleic acid sequence SEQ ID NO. 12070, preferably the coding region thereof, conferred the production of or the increase in tyrosine compared with the wild type control.

The nucleic acid sequence of Slr1739 from Synechocystis sp., e.g. as shown in the respective line in column 5 of Table I, application no. 3, has been published in C. And the activity of the gene product thereof is the activity of photosystem II protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “photosystem II protein”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr1739, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr1739, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr1739, or a functional equivalent or a homolog thereof as depicted in column 8 of Table II, application no. 3, preferably a homolog or functional equivalent as depicted in column 8 of Table II B, application no. 3, and being depicted in the same respective line as said Slr1739, and preferably the activity is increased plastidic.

Accordingly, in one embodiment, the molecule which activity is to be increased in the process of the invention is the gene product with an activity as a “photosystem II protein”, preferably it is the molecule of section (a) or (b) of this paragraph.

In particular, it was observed that in plants, especially in Arabidopsis thaliana, increasing or generating the activity of a gene product plastidic with the activity of a “photosystem II protein”, preferably being encoded by a gene comprising the nucleic acid sequence SEQ ID NO. 58731, preferably the coding region thereof, conferred the production of or the increase in phenylalanine compared with the wild type control.

The nucleic acid sequence of Slr1739 from Synechocystis sp., e.g. as shown in the respective line in column 5 of Table I, application no. 3, has been published in C. And the activity of the gene product thereof is the activity of photosystem II protein.

Accordingly, in one embodiment, the process of the present invention for producing serine in a non-human organism, like a microorganism or a plant or a part thereof, comprises increasing or generating the activity of a gene product with the activity of a gene product conferring the activity of “photosystem II protein”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr1739, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr1739, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr1739, or a functional equivalent or a homolog thereof as depicted in column 8 of Table II, application no. 3, preferably a homolog or functional equivalent as depicted in column 8 of Table II B, application no. 3, and being depicted in the same respective line as said Slr1739, and preferably the activity is increased plastidic.

Accordingly, in one embodiment, the molecule which activity is to be increased in the process of the invention is the gene product with an activity as a “photosystem II protein”, preferably it is the molecule of section (a) or (b) of this paragraph.

In particular, it was observed that in plants, especially in Arabidopsis thaliana, increasing or generating the activity of a gene product plastidic with the activity of a “photosystem II protein”, preferably being encoded by a gene comprising the nucleic acid sequence SEQ ID NO. 58731, preferably the coding region thereof, conferred the production of or the increase in serine compared with the wild type control.

The nucleic acid sequence of Slr1739 from Synechocystis sp., e.g. as shown in the respective line in column 5 of Table I, application no. 3, has been published in C. And the activity of the gene product thereof is the activity of photosystem II protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “photosystem II protein”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr1739, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr1739, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr1739, or a functional equivalent or a homolog thereof as depicted in column 8 of Table II, application no. 3, preferably a homolog or functional equivalent as depicted in column 8 of Table II B, application no. 3, and being depicted in the same respective line as said Slr1739, and preferably the activity is increased plastidic.

Accordingly, in one embodiment, the molecule which activity is to be increased in the process of the invention is the gene product with an activity as a “photosystem II protein”, preferably it is the molecule of section (a) or (b) of this paragraph.

In particular, it was observed that in plants, especially in Arabidopsis thaliana, increasing or generating the activity of a gene product plastidic with the activity of a “photosystem II protein”, preferably being encoded by a gene comprising the nucleic acid sequence SEQ ID NO. 58731, preferably the coding region thereof, conferred the production of or the increase in tyrosine compared with the wild type control.

The nucleic acid sequence of Slr1739 from Synechocystis sp., e.g. as shown in the respective line in column 5 of Table I, application no. 3, has been published in C. And the activity of the gene product thereof is the activity of photosystem II protein.

Accordingly, in one embodiment, the process of the present invention for producing 5oxoproline in a non-human organism, like a microorganism or a plant or a part thereof, comprises increasing or generating the activity of a gene product with the activity of a gene product conferring the activity of “photosystem II protein”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr1739, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr1739, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr1739, or a functional equivalent or a homolog thereof as depicted in column 8 of Table II, application no. 3, preferably a homolog or functional equivalent as depicted in column 8 of Table II B, application no. 3, and being depicted in the same respective line as said Slr1739, and preferably the activity is increased plastidic.

Accordingly, in one embodiment, the molecule which activity is to be increased in the process of the invention is the gene product with an activity as a “photosystem II protein”, preferably it is the molecule of section (a) or (b) of this paragraph.

In particular, it was observed that in plants, especially in Arabidopsis thaliana, increasing or generating the activity of a gene product plastidic with the activity of a “photosystem II protein”, preferably being encoded by a gene comprising the nucleic acid sequence SEQ ID NO. 58731, preferably the coding region thereof, conferred the production of or the increase in 5-oxoproline compared with the wild type control.

The nucleic acid sequence of Slr1742 from Synechocystis sp., e.g. as shown in the respective line in column 5 of Table I, application no. 3, has been published in C. And the activity of the gene product thereof is the activity of glutamine amidotransferase.

Accordingly, in one embodiment, the process of the present invention for producing asparagine in a non-human organism, like a microorganism or a plant or a part thereof, comprises increasing or generating the activity of a gene product with the activity of a gene product conferring the activity of “glutamine amidotransferase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr1742, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr1742, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr1742, or a functional equivalent or a homolog thereof as depicted in column 8 of Table II, application no. 3, preferably a homolog or functional equivalent as depicted in column 8 of Table II B, application no. 3, and being depicted in the same respective line as said Slr1742, and preferably the activity is increased mitochondrial.

Accordingly, in one embodiment, the molecule which activity is to be increased in the process of the invention is the gene product with an activity as a “glutamine amidotransferase”, preferably it is the molecule of section (a) or (b) of this paragraph.

In particular, it was observed that in plants, especially in Arabidopsis thaliana, increasing or generating the activity of a gene product mitochondrial with the activity of a “glutamine amidotransferase”, preferably being encoded by a gene comprising the nucleic acid sequence SEQ ID NO. 58751, preferably the coding region thereof, conferred the production of or the increase in asparagine compared with the wild type control.

The nucleic acid sequence of Slr1791 from Synechocystis sp., e.g. as shown in the respective line in column 5 of Table I, application no. 3, has been published in C. And the activity of the gene product thereof is the activity of phosphoadenosine phosphosulfate reductase .

Accordingly, in one embodiment, the process of the present invention for producing citrulline in a non-human organism, like a microorganism or a plant or a part thereof, comprises increasing or generating the activity of a gene product with the activity of a gene product conferring the activity of “phosphoadenosine phosphosulfate reductase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr1791, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr1791, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr1791, or a functional equivalent or a homolog thereof as depicted in column 8 of Table II, application no. 3, preferably a homolog or functional equivalent as depicted in column 8 of Table II B, application no. 3, and being depicted in the same respective line as said Slr1791, and preferably the activity is increased mitochondrial.

Accordingly, in one embodiment, the molecule which activity is to be increased in the process of the invention is the gene product with an activity as a “phosphoadenosine phosphosulfate reductase”, preferably it is the molecule of section (a) or (b) of this paragraph.

In particular, it was observed that in plants, especially in Arabidopsis thaliana, increasing or generating the activity of a gene product mitochondrial with the activity of a “phosphoadenosine phosphosulfate reductase”, preferably being encoded by a gene comprising the nucleic acid sequence SEQ ID NO. 12140, preferably the coding region thereof, conferred the production of or the increase in citrulline compared with the wild type control.

The nucleic acid sequence of Slr1882 from Synechocystis sp., e.g. as shown in the respective line in column 5 of Table I, application no. 3, has been published in C. And the activity of the gene product thereof is the activity of riboflavin biosynthesis protein.

Accordingly, in one embodiment, the process of the present invention for producing asparagine in a non-human organism, like a microorganism or a plant or a part thereof, comprises increasing or generating the activity of a gene product with the activity of a gene product conferring the activity of “riboflavin biosynthesis protein”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr1882, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr1882, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr1882, or a functional equivalent or a homolog thereof as depicted in column 8 of Table II, application no. 3, preferably a homolog or functional equivalent as depicted in column 8 of Table II B, application no. 3, and being depicted in the same respective line as said Slr1882, and preferably the activity is increased non-targeted.

Accordingly, in one embodiment, the molecule which activity is to be increased in the process of the invention is the gene product with an activity as a “riboflavin biosynthesis protein”, preferably it is the molecule of section (a) or (b) of this paragraph.

In particular, it was observed that in plants, especially in Arabidopsis thaliana, increasing or generating the activity of a gene product non-targeted with the activity of a “riboflavin biosynthesis protein”, preferably being encoded by a gene comprising the nucleic acid sequence SEQ ID NO. 59041, preferably the coding region thereof, conferred the production of or the increase in asparagine compared with the wild type control.

The nucleic acid sequence of Slr2072 from Synechocystis sp., e.g. as shown in the respective line in column 5 of Table I, application no. 3, has been published in C. And the activity of the gene product thereof is the activity of threonine dehydratase.

Accordingly, in one embodiment, the process of the present invention for producing homoserine in a non-human organism, like a microorganism or a plant or a part thereof, comprises increasing or generating the activity of a gene product with the activity of a gene product conferring the activity of “threonine dehydratase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr2072, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr2072, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr2072, or a functional equivalent or a homolog thereof as depicted in column 8 of Table II, application no. 3, preferably a homolog or functional equivalent as depicted in column 8 of Table II B, application no. 3, and being depicted in the same respective line as said Slr2072, and preferably the activity is increased plastidic.

Accordingly, in one embodiment, the molecule which activity is to be increased in the process of the invention is the gene product with an activity as a “threonine dehydratase”, preferably it is the molecule of section (a) or (b) of this paragraph.

In particular, it was observed that in plants, especially in Arabidopsis thaliana, increasing or generating the activity of a gene product plastidic with the activity of a “threonine dehydratase”, preferably being encoded by a gene comprising the nucleic acid sequence SEQ ID NO. 12341, preferably the coding region thereof, conferred the production of or the increase in homoserine compared with the wild type control.

The nucleic acid sequence of Slr2072 from Synechocystis sp., e.g. as shown in the respective line in column 5 of Table I, application no. 3, has been published in C. And the activity of the gene product thereof is the activity of threonine dehydratase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “threonine dehydratase”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr2072, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr2072, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr2072, or a functional equivalent or a homolog thereof as depicted in column 8 of Table II, application no. 3, preferably a homolog or functional equivalent as depicted in column 8 of Table II B, application no. 3, and being depicted in the same respective line as said Slr2072, and preferably the activity is increased mitochondrial.

Accordingly, in one embodiment, the molecule which activity is to be increased in the process of the invention is the gene product with an activity as a “threonine dehydratase”, preferably it is the molecule of section (a) or (b) of this paragraph.

In particular, it was observed that in plants, especially in Arabidopsis thaliana, increasing or generating the activity of a gene product mitochondrial with the activity of a “threonine dehydratase”, preferably being encoded by a gene comprising the nucleic acid sequence SEQ ID NO. 12341, preferably the coding region thereof, conferred the production of or the increase in tyrosine compared with the wild type control.

The nucleic acid sequence of Slr2124 from Synechocystis sp., e.g. as shown in the respective line in column 5 of Table I, application no. 3, has been published in C. And the activity of the gene product thereof is the activity of short-chain alcohol dehydrogenase family.

Accordingly, in one embodiment, the process of the present invention for producing alanine in a non-human organism, like a microorganism or a plant or a part thereof, comprises increasing or generating the activity of a gene product with the activity of a gene product conferring the activity of “short-chain alcohol dehydrogenase family”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr2124, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr2124, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr2124, or a functional equivalent or a homolog thereof as depicted in column 8 of Table II, application no. 3, preferably a homolog or functional equivalent as depicted in column 8 of Table II B, application no. 3, and being depicted in the same respective line as said Slr2124, and preferably the activity is increased plastidic.

Accordingly, in one embodiment, the molecule which activity is to be increased in the process of the invention is the gene product with an activity as a “short-chain alcohol dehydrogenase family”, preferably it is the molecule of section (a) or (b) of this paragraph.

In particular, it was observed that in plants, especially in Arabidopsis thaliana, increasing or generating the activity of a gene product plastidic with the activity of a “short-chain alcohol dehydrogenase family”, preferably being encoded by a gene comprising the nucleic acid sequence SEQ ID NO. 59370, preferably the coding region thereof, conferred the production of or the increase in alanine compared with the wild type control.

The nucleic acid sequence of Slr2124 from Synechocystis sp., e.g. as shown in the respective line in column 5 of Table I, application no. 3, has been published in C. And the activity of the gene product thereof is the activity of short-chain alcohol dehydrogenase family.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “short-chain alcohol dehydrogenase family”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr2124, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr2124, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr2124, or a functional equivalent or a homolog thereof as depicted in column 8 of Table II, application no. 3, preferably a homolog or functional equivalent as depicted in column 8 of Table II B, application no. 3, and being depicted in the same respective line as said Slr2124, and preferably the activity is increased plastidic.

Accordingly, in one embodiment, the molecule which activity is to be increased in the process of the invention is the gene product with an activity as a “short-chain alcohol dehydrogenase family”, preferably it is the molecule of section (a) or (b) of this paragraph.

In particular, it was observed that in plants, especially in Arabidopsis thaliana, increasing or generating the activity of a gene product plastidic with the activity of a “short-chain alcohol dehydrogenase family”, preferably being encoded by a gene comprising the nucleic acid sequence SEQ ID NO. 59370, preferably the coding region thereof, conferred the production of or the increase in phenylalanine compared with the wild type control.

The nucleic acid sequence of Slr2124 from Synechocystis sp., e.g. as shown in the respective line in column 5 of Table I, application no. 3, has been published in C. And the activity of the gene product thereof is the activity of short-chain alcohol dehydrogenase family.

Accordingly, in one embodiment, the process of the present invention for producing tyrosine in a non-human organism, like a microorganism or a plant or a part thereof, comprises increasing or generating the activity of a gene product with the activity of a gene product conferring the activity of “short-chain alcohol dehydrogenase family”, especially from Synechocystis sp. or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Slr2124, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Slr2124, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Slr2124, or a functional equivalent or a homolog thereof as depicted in column 8 of Table II, application no. 3, preferably a homolog or functional equivalent as depicted in column 8 of Table II B, application no. 3, and being depicted in the same respective line as said Slr2124, and preferably the activity is increased plastidic.

Accordingly, in one embodiment, the molecule which activity is to be increased in the process of the invention is the gene product with an activity as a “short-chain alcohol dehydrogenase family”, preferably it is the molecule of section (a) or (b) of this paragraph.

In particular, it was observed that in plants, especially in Arabidopsis thaliana, increasing or generating the activity of a gene product plastidic with the activity of a “short-chain alcohol dehydrogenase family”, preferably being encoded by a gene comprising the nucleic acid sequence SEQ ID NO. 59370, preferably the coding region thereof, conferred the production of or the increase in tyrosine compared with the wild type control.

The nucleic acid sequence of TTC0019 from Thermus thermophilus, e.g. as shown in the respective line in column 5 of Table I, application no. 3, has been published in E. And the activity of the gene product thereof is the activity of Sec-independent protein translocase subunit.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “Sec-independent protein translocase subunit”, especially from Thermus thermophilus or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said TTC0019, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said TTC0019, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said TTC0019, or a functional equivalent or a homolog thereof as depicted in column 8 of Table II, application no. 3, preferably a homolog or functional equivalent as depicted in column 8 of Table II B, application no. 3, and being depicted in the same respective line as said TTC0019, and preferably the activity is increased non-targeted.

Accordingly, in one embodiment, the molecule which activity is to be increased in the process of the invention is the gene product with an activity as a “Sec-independent protein translocase subunit”, preferably it is the molecule of section (a) or (b) of this paragraph.

In particular, it was observed that in plants, especially in Arabidopsis thaliana, increasing or generating the activity of a gene product non-targeted with the activity of a “Sec-independent protein translocase subunit”, preferably being encoded by a gene comprising the nucleic acid sequence SEQ ID NO. 12698, preferably the coding region thereof, conferred the production of or the increase in phenylalanine compared with the wild type control.

The nucleic acid sequence of TTC0019 from Thermus thermophilus, e.g. as shown in the respective line in column 5 of Table I, application no. 3, has been published in E. And the activity of the gene product thereof is the activity of Sec-independent protein translocase subunit.

Accordingly, in one embodiment, the process of the present invention for producing tyrosine in a non-human organism, like a microorganism or a plant or a part thereof, comprises increasing or generating the activity of a gene product with the activity of a gene product conferring the activity of “Sec-independent protein translocase subunit”, especially from Thermus thermophilus or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said TTC0019, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said TTC0019, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said TTC0019, or a functional equivalent or a homolog thereof as depicted in column 8 of Table II, application no. 3, preferably a homolog or functional equivalent as depicted in column 8 of Table II B, application no. 3, and being depicted in the same respective line as said TTC0019, and preferably the activity is increased non-targeted.

Accordingly, in one embodiment, the molecule which activity is to be increased in the process of the invention is the gene product with an activity as a “Sec-independent protein translocase subunit”, preferably it is the molecule of section (a) or (b) of this paragraph.

In particular, it was observed that in plants, especially in Arabidopsis thaliana, increasing or generating the activity of a gene product non-targeted with the activity of a “Sec-independent protein translocase subunit”, preferably being encoded by a gene comprising the nucleic acid sequence SEQ ID NO. 12698, preferably the coding region thereof, conferred the production of or the increase in tyrosine compared with the wild type control.

The nucleic acid sequence of TTC0019 from Thermus thermophilus, e.g. as shown in the respective line in column 5 of Table I, application no. 3, has been published in E. And the activity of the gene product thereof is the activity of Sec-independent protein translocase subunit.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “Sec-independent protein translocase subunit”, especially from Thermus thermophilus or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said TTC0019, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said TTC0019, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said TTC0019, or a functional equivalent or a homolog thereof as depicted in column 8 of Table II, application no. 3, preferably a homolog or functional equivalent as depicted in column 8 of Table II B, application no. 3, and being depicted in the same respective line as said TTC0019, and preferably the activity is increased non-targeted.

Accordingly, in one embodiment, the molecule which activity is to be increased in the process of the invention is the gene product with an activity as a “Sec-independent protein translocase subunit”, preferably it is the molecule of section (a) or (b) of this paragraph.

In particular, it was observed that in plants, especially in Arabidopsis thaliana, increasing or generating the activity of a gene product non-targeted with the activity of a “Sec-independent protein translocase subunit”, preferably being encoded by a gene comprising the nucleic acid sequence SEQ ID NO. 12698, preferably the coding region thereof, conferred the production of or the increase in 5-oxoproline compared with the wild type control.

The nucleic acid sequence of TTC0239 from Thermus thermophilus, e.g. as shown in the respective line in column 5 of Table I, application no. 3, has been published in E. And the activity of the gene product thereof is the activity of transcriptional regulator.

Accordingly, in one embodiment, the process of the present invention for producing tyrosine in a non-human organism, like a microorganism or a plant or a part thereof, comprises increasing or generating the activity of a gene product with the activity of a gene product conferring the activity of “transcriptional regulator”, especially from Thermus thermophilus or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said TTC0239, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said TTC0239, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said TTC0239, or a functional equivalent or a homolog thereof as depicted in column 8 of Table II, application no. 3, preferably a homolog or functional equivalent as depicted in column 8 of Table II B, application no. 3, and being depicted in the same respective line as said TTC0239, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 88526, preferably the coding region thereof, conferred the production of or the increase in tyrosine compared with the wild type control.

The nucleic acid sequence of TTC0337 from Thermus thermophilus, e.g. as shown in the respective line in column 5 of Table I, application no. 3, has been published in E. And the activity of the gene product thereof is the activity of amino acid ABC transporter permease protein.

Accordingly, in one embodiment, the process of the present invention for producing asparagine in a non-human organism, like a microorganism or a plant or a part thereof, comprises increasing or generating the activity of a gene product with the activity of a gene product conferring the activity of “amino acid ABC transporter permease protein”, especially from Thermus thermophilus or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said TTC0337, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said TTC0337, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said TTC0337, or a functional equivalent or a homolog thereof as depicted in column 8 of Table II, application no. 3, preferably a homolog or functional equivalent as depicted in column 8 of Table II B, application no. 3, and being depicted in the same respective line as said TTC0337, and preferably the activity is increased non-targeted.

Accordingly, in one embodiment, the molecule which activity is to be increased in the process of the invention is the gene product with an activity as a “amino acid ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “amino acid ABC transporter permease protein”, preferably being encoded by a gene comprising the nucleic acid sequence SEQ ID NO. 61070, preferably the coding region thereof, conferred the production of or the increase in asparagine compared with the wild type control.

The nucleic acid sequence of TTC0768 from Thermus thermophilus, e.g. as shown in the respective line in column 5 of Table I, application no. 3, has been published in E. And the activity of the gene product thereof is the activity of TTC0768-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “TTC0768-protein”, especially from Thermus thermophilus or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said TTC0768, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said TTC0768, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said TTC0768, or a functional equivalent or a homolog thereof as depicted in column 8 of Table II, application no. 3, preferably a homolog or functional equivalent as depicted in column 8 of Table II B, application no. 3, and being depicted in the same respective line as said TTC0768, and preferably the activity is increased non-targeted.

Accordingly, in one embodiment, the molecule which activity is to be increased in the process of the invention is the gene product with an activity as a “TTC0768-protein”, preferably it is the molecule of section (a) or (b) of this paragraph.

In particular, it was observed that in plants, especially in Arabidopsis thaliana, increasing or generating the activity of a gene product non-targeted with the activity of a “TTC0768-protein”, preferably being encoded by a gene comprising the nucleic acid sequence SEQ ID NO. 61532, preferably the coding region thereof, conferred the production of or the increase in tyrosine compared with the wild type control.

The nucleic acid sequence of TTC0881 from Thermus thermophilus, e.g. as shown in the respective line in column 5 of Table I, application no. 3, has been published in E. And the activity of the gene product thereof is the activity of TTC0881-protein.

Accordingly, in one embodiment, the process of the present invention for producing alanine in a non-human organism, like a microorganism or a plant or a part thereof, comprises increasing or generating the activity of a gene product with the activity of a gene product conferring the activity of “TTC0881-protein”, especially from Thermus thermophilus or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said TTC0881, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said TTC0881, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said TTC0881, or a functional equivalent or a homolog thereof as depicted in column 8 of Table II, application no. 3, preferably a homolog or functional equivalent as depicted in column 8 of Table II B, application no. 3, and being depicted in the same respective line as said TTC0881, and preferably the activity is increased non-targeted.

Accordingly, in one embodiment, the molecule which activity is to be increased in the process of the invention is the gene product with an activity as a “TTC0881-protein”, preferably it is the molecule of section (a) or (b) of this paragraph.

In particular, it was observed that in plants, especially in Arabidopsis thaliana, increasing or generating the activity of a gene product non-targeted with the activity of a “TTC0881-protein”, preferably being encoded by a gene comprising the nucleic acid sequence SEQ ID NO. 88875, preferably the coding region thereof, conferred the production of or the increase in alanine compared with the wild type control.

The nucleic acid sequence of TTC0881 from Thermus thermophilus, e.g. as shown in the respective line in column 5 of Table I, application no. 3, has been published in E. And the activity of the gene product thereof is the activity of TTC0881-protein.

Accordingly, in one embodiment, the process of the present invention for producing serine in a non-human organism, like a microorganism or a plant or a part thereof, comprises increasing or generating the activity of a gene product with the activity of a gene product conferring the activity of “TTC0881-protein”, especially from Thermus thermophilus or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said TTC0881, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said TTC0881, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said TTC0881, or a functional equivalent or a homolog thereof as depicted in column 8 of Table II, application no. 3, preferably a homolog or functional equivalent as depicted in column 8 of Table II B, application no. 3, and being depicted in the same respective line as said TTC0881, and preferably the activity is increased non-targeted.

Accordingly, in one embodiment, the molecule which activity is to be increased in the process of the invention is the gene product with an activity as a “TTC0881-protein”, preferably it is the molecule of section (a) or (b) of this paragraph.

In particular, it was observed that in plants, especially in Arabidopsis thaliana, increasing or generating the activity of a gene product non-targeted with the activity of a “TTC0881-protein”, preferably being encoded by a gene comprising the nucleic acid sequence SEQ ID NO. 88875, preferably the coding region thereof, conferred the production of or the increase in serine compared with the wild type control.

The nucleic acid sequence of TTC0917 from Thermus thermophilus, e.g. as shown in the respective line in column 5 of Table I, application no. 3, has been published in E. And the activity of the gene product thereof is the activity of metal-dependent hydrolase.

Accordingly, in one embodiment, the process of the present invention for producing alanine in a non-human organism, like a microorganism or a plant or a part thereof, comprises increasing or generating the activity of a gene product with the activity of a gene product conferring the activity of “metal-dependent hydrolase”, especially from Thermus thermophilus or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said TTC0917, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said TTC0917, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said TTC0917, or a functional equivalent or a homolog thereof as depicted in column 8 of Table II, application no. 3, preferably a homolog or functional equivalent as depicted in column 8 of Table II B, application no. 3, and being depicted in the same respective line as said TTC0917, and preferably the activity is increased non-targeted.

Accordingly, in one embodiment, the molecule which activity is to be increased in the process of the invention is the gene product with an activity as a “metal-dependent hydrolase”, preferably it is the molecule of section (a) or (b) of this paragraph.

In particular, it was observed that in plants, especially in Arabidopsis thaliana, increasing or generating the activity of a gene product non-targeted with the activity of a “metal-dependent hydrolase”, preferably being encoded by a gene comprising the nucleic acid sequence SEQ ID NO. 61553, preferably the coding region thereof, conferred the production of or the increase in alanine compared with the wild type control.

The nucleic acid sequence of TTC0917 from Thermus thermophilus, e.g. as shown in the respective line in column 5 of Table I, application no. 3, has been published in E. And the activity of the gene product thereof is the activity of metal-dependent hydrolase.

Accordingly, in one embodiment, the process of the present invention for producing serine in a non-human organism, like a microorganism or a plant or a part thereof, comprises increasing or generating the activity of a gene product with the activity of a gene product conferring the activity of “metal-dependent hydrolase”, especially from Thermus thermophilus or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said TTC0917, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said TTC0917, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said TTC0917, or a functional equivalent or a homolog thereof as depicted in column 8 of Table II, application no. 3, preferably a homolog or functional equivalent as depicted in column 8 of Table II B, application no. 3, and being depicted in the same respective line as said TTC0917, and preferably the activity is increased non-targeted.

Accordingly, in one embodiment, the molecule which activity is to be increased in the process of the invention is the gene product with an activity as a “metal-dependent hydrolase”, preferably it is the molecule of section (a) or (b) of this paragraph.

In particular, it was observed that in plants, especially in Arabidopsis thaliana, increasing or generating the activity of a gene product non-targeted with the activity of a “metal-dependent hydrolase”, preferably being encoded by a gene comprising the nucleic acid sequence SEQ ID NO. 61553, preferably the coding region thereof, conferred the production of or the increase in serine compared with the wild type control.

The nucleic acid sequence of TTC1193 from Thermus thermophilus, e.g. as shown in the respective line in column 5 of Table I, application no. 3, has been published in E. And the activity of the gene product thereof is the activity of multiple antibiotic resistance protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “multiple antibiotic resistance protein”, especially from Thermus thermophilus or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said TTC1193, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said TTC1193, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said TTC1193, or a functional equivalent or a homolog thereof as depicted in column 8 of Table II, application no. 3, preferably a homolog or functional equivalent as depicted in column 8 of Table II B, application no. 3, and being depicted in the same respective line as said TTC1193, and preferably the activity is increased non-targeted.

Accordingly, in one embodiment, the molecule which activity is to be increased in the process of the invention is the gene product with an activity as a “multiple antibiotic resistance protein”, preferably it is the molecule of section (a) or (b) of this paragraph.

In particular, it was observed that in plants, especially in Arabidopsis thaliana, increasing or generating the activity of a gene product non-targeted with the activity of a “multiple antibiotic resistance protein”, preferably being encoded by a gene comprising the nucleic acid sequence SEQ ID NO. 61723, preferably the coding region thereof, conferred the production of or the increase in phenylalanine compared with the wild type control.

The nucleic acid sequence of TTC1193 from Thermus thermophilus, e.g. as shown in the respective line in column 5 of Table I, application no. 3, has been published in E. And the activity of the gene product thereof is the activity of multiple antibiotic resistance protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “multiple antibiotic resistance protein”, especially from Thermus thermophilus or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said TTC1193, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said TTC1193, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said TTC1193, or a functional equivalent or a homolog thereof as depicted in column 8 of Table II, application no. 3, preferably a homolog or functional equivalent as depicted in column 8 of Table II B, application no. 3, and being depicted in the same respective line as said TTC1193, and preferably the activity is increased non-targeted.

Accordingly, in one embodiment, the molecule which activity is to be increased in the process of the invention is the gene product with an activity as a “multiple antibiotic resistance protein”, preferably it is the molecule of section (a) or (b) of this paragraph.

In particular, it was observed that in plants, especially in Arabidopsis thaliana, increasing or generating the activity of a gene product non-targeted with the activity of a “multiple antibiotic resistance protein”, preferably being encoded by a gene comprising the nucleic acid sequence SEQ ID NO. 61723, preferably the coding region thereof, conferred the production of or the increase in tyrosine compared with the wild type control.

The nucleic acid sequence of TTC1386 from Thermus thermophilus, e.g. as shown in the respective line in column 5 of Table I, application no. 3, has been published in E. And the activity of the gene product thereof is the activity of TTC1386-protein.

Accordingly, in one embodiment, the process of the present invention for producing alanine in a non-human organism, like a microorganism or a plant or a part thereof, comprises increasing or generating the activity of a gene product with the activity of a gene product conferring the activity of “TTC1386-protein”, especially from Thermus thermophilus or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said TTC1386, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said TTC1386, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said TTC1386, or a functional equivalent or a homolog thereof as depicted in column 8 of Table II, application no. 3, preferably a homolog or functional equivalent as depicted in column 8 of Table II B, application no. 3, and being depicted in the same respective line as said TTC1386, and preferably the activity is increased non-targeted.

Accordingly, in one embodiment, the molecule which activity is to be increased in the process of the invention is the gene product with an activity as a “TTC1386-protein”, preferably it is the molecule of section (a) or (b) of this paragraph.

In particular, it was observed that in plants, especially in Arabidopsis thaliana, increasing or generating the activity of a gene product non-targeted with the activity of a “TTC1386-protein”, preferably being encoded by a gene comprising the nucleic acid sequence SEQ ID NO. 62079, preferably the coding region thereof, conferred the production of or the increase in alanine compared with the wild type control.

The nucleic acid sequence of TTC1386 from Thermus thermophilus, e.g. as shown in the respective line in column 5 of Table I, application no. 3, has been published in E. And the activity of the gene product thereof is the activity of TTC1386-protein.

Accordingly, in one embodiment, the process of the present invention for producing glycine in a non-human organism, like a microorganism or a plant or a part thereof, comprises increasing or generating the activity of a gene product with the activity of a gene product conferring the activity of “TTC1386-protein”, especially from Thermus thermophilus or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said TTC1386, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said TTC1386, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said TTC1386, or a functional equivalent or a homolog thereof as depicted in column 8 of Table II, application no. 3, preferably a homolog or functional equivalent as depicted in column 8 of Table II B, application no. 3, and being depicted in the same respective line as said TTC1386, and preferably the activity is increased non-targeted.

Accordingly, in one embodiment, the molecule which activity is to be increased in the process of the invention is the gene product with an activity as a “TTC1386-protein”, preferably it is the molecule of section (a) or (b) of this paragraph.

In particular, it was observed that in plants, especially in Arabidopsis thaliana, increasing or generating the activity of a gene product non-targeted with the activity of a “TTC1386-protein”, preferably being encoded by a gene comprising the nucleic acid sequence SEQ ID NO. 62079, preferably the coding region thereof, conferred the production of or the increase in glycine compared with the wild type control.

The nucleic acid sequence of TTC1386 from Thermus thermophilus, e.g. as shown in the respective line in column 5 of Table I, application no. 3, has been published in E. And the activity of the gene product thereof is the activity of TTC1386-protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “TTC1386-protein”, especially from Thermus thermophilus or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said TTC1386, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said TTC1386, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said TTC1386, or a functional equivalent or a homolog thereof as depicted in column 8 of Table II, application no. 3, preferably a homolog or functional equivalent as depicted in column 8 of Table II B, application no. 3, and being depicted in the same respective line as said TTC1386, and preferably the activity is increased non-targeted.

Accordingly, in one embodiment, the molecule which activity is to be increased in the process of the invention is the gene product with an activity as a “TTC1386-protein”, preferably it is the molecule of section (a) or (b) of this paragraph.

In particular, it was observed that in plants, especially in Arabidopsis thaliana, increasing or generating the activity of a gene product non-targeted with the activity of a “TTC1386-protein”, preferably being encoded by a gene comprising the nucleic acid sequence SEQ ID NO. 62079, preferably the coding region thereof, conferred the production of or the increase in phenylalanine compared with the wild type control.

The nucleic acid sequence of TTC1386 from Thermus thermophilus, e.g. as shown in the respective line in column 5 of Table I, application no. 3, has been published in E. And the activity of the gene product thereof is the activity of TTC1386-protein.

Accordingly, in one embodiment, the process of the present invention for producing serine in a non-human organism, like a microorganism or a plant or a part thereof, comprises increasing or generating the activity of a gene product with the activity of a gene product conferring the activity of “TTC1386-protein”, especially from Thermus thermophilus or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said TTC1386, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said TTC1386, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said TTC1386, or a functional equivalent or a homolog thereof as depicted in column 8 of Table II, application no. 3, preferably a homolog or functional equivalent as depicted in column 8 of Table II B, application no. 3, and being depicted in the same respective line as said TTC1386, and preferably the activity is increased non-targeted.

Accordingly, in one embodiment, the molecule which activity is to be increased in the process of the invention is the gene product with an activity as a “TTC1386-protein”, preferably it is the molecule of section (a) or (b) of this paragraph.

In particular, it was observed that in plants, especially in Arabidopsis thaliana, increasing or generating the activity of a gene product non-targeted with the activity of a “TTC1386-protein”, preferably being encoded by a gene comprising the nucleic acid sequence SEQ ID NO. 62079, preferably the coding region thereof, conferred the production of or the increase in serine compared with the wild type control.

The nucleic acid sequence of TTC1386 from Thermus thermophilus, e.g. as shown in the respective line in column 5 of Table I, application no. 3, has been published in E. And the activity of the gene product thereof is the activity of TTC1386-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “TTC1386-protein”, especially from Thermus thermophilus or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said TTC1386, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said TTC1386, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said TTC1386, or a functional equivalent or a homolog thereof as depicted in column 8 of Table II, application no. 3, preferably a homolog or functional equivalent as depicted in column 8 of Table II B, application no. 3, and being depicted in the same respective line as said TTC1386, and preferably the activity is increased non-targeted.

Accordingly, in one embodiment, the molecule which activity is to be increased in the process of the invention is the gene product with an activity as a “TTC1386-protein”, preferably it is the molecule of section (a) or (b) of this paragraph.

In particular, it was observed that in plants, especially in Arabidopsis thaliana, increasing or generating the activity of a gene product non-targeted with the activity of a “TTC1386-protein”, preferably being encoded by a gene comprising the nucleic acid sequence SEQ ID NO. 62079, preferably the coding region thereof, conferred the production of or the increase in tyrosine compared with the wild type control.

The nucleic acid sequence of TTC1550 from Thermus thermophilus, e.g. as shown in the respective line in column 5 of Table I, application no. 3, has been published in E. And the activity of the gene product thereof is the activity of homocitrate synthase.

Accordingly, in one embodiment, the process of the present invention for producing alanine in a non-human organism, like a microorganism or a plant or a part thereof, comprises increasing or generating the activity of a gene product with the activity of a gene product conferring the activity of “homocitrate synthase”, especially from Thermus thermophilus or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said TTC1550, or a functional equivalent or a homolog thereof as shown in column 8 of Table

I, application no. 3, preferably the coding region thereof, particularly a homolog or functional equivalent as shown in column 8 of Table I B, application no. 3, and being depicted in the same respective line as said TTC1550, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said TTC1550, or a functional equivalent or a homolog thereof as depicted in column 8 of Table II, application no. 3, preferably a homolog or functional equivalent as depicted in column 8 of Table II B, application no. 3, and being depicted in the same respective line as said TTC1550, and preferably the activity is increased non-targeted.

Accordingly, in one embodiment, the molecule which activity is to be increased in the process of the invention is the gene product with an activity as a “homocitrate synthase”, preferably it is the molecule of section (a) or (b) of this paragraph.

In particular, it was observed that in plants, especially in Arabidopsis thaliana, increasing or generating the activity of a gene product non-targeted with the activity of a “homocitrate synthase”, preferably being encoded by a gene comprising the nucleic acid sequence SEQ ID NO. 12974, preferably the coding region thereof, conferred the production of or the increase in alanine compared with the wild type control.

The nucleic acid sequence of TTC1550 from Thermus thermophilus, e.g. as shown in the respective line in column 5 of Table I, application no. 3, has been published in E. And the activity of the gene product thereof is the activity of homocitrate synthase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “homocitrate synthase”, especially from Thermus thermophilus or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said TTC1550, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said TTC1550, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said TTC1550, or a functional equivalent or a homolog thereof as depicted in column 8 of Table II, application no. 3, preferably a homolog or functional equivalent as depicted in column 8 of Table II B, application no. 3, and being depicted in the same respective line as said TTC1550, and preferably the activity is increased non-targeted.

Accordingly, in one embodiment, the molecule which activity is to be increased in the process of the invention is the gene product with an activity as a “homocitrate synthase”, preferably it is the molecule of section (a) or (b) of this paragraph.

In particular, it was observed that in plants, especially in Arabidopsis thaliana, increasing or generating the activity of a gene product non-targeted with the activity of a “homocitrate synthase”, preferably being encoded by a gene comprising the nucleic acid sequence SEQ ID NO. 12974, preferably the coding region thereof, conferred the production of or the increase in phenylalanine compared with the wild type control.

The nucleic acid sequence of TTC1550 from Thermus thermophilus, e.g. as shown in the respective line in column 5 of Table I, application no. 3, has been published in E. And the activity of the gene product thereof is the activity of homocitrate synthase.

Accordingly, in one embodiment, the process of the present invention for producing serine in a non-human organism, like a microorganism or a plant or a part thereof, comprises increasing or generating the activity of a gene product with the activity of a gene product conferring the activity of “homocitrate synthase”, especially from Thermus thermophilus or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said TTC1550, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said TTC1550, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said TTC1550, or a functional equivalent or a homolog thereof as depicted in column 8 of Table II, application no. 3, preferably a homolog or functional equivalent as depicted in column 8 of Table II B, application no. 3, and being depicted in the same respective line as said TTC1550, and preferably the activity is increased non-targeted.

Accordingly, in one embodiment, the molecule which activity is to be increased in the process of the invention is the gene product with an activity as a “homocitrate synthase”, preferably it is the molecule of section (a) or (b) of this paragraph.

In particular, it was observed that in plants, especially in Arabidopsis thaliana, increasing or generating the activity of a gene product non-targeted with the activity of a “homocitrate synthase”, preferably being encoded by a gene comprising the nucleic acid sequence SEQ ID NO. 12974, preferably the coding region thereof, conferred the production of or the increase in serine compared with the wild type control.

The nucleic acid sequence of TTC1550 from Thermus thermophilus, e.g. as shown in the respective line in column 5 of Table I, application no. 3, has been published in E. And the activity of the gene product thereof is the activity of homocitrate synthase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “homocitrate synthase”, especially from Thermus thermophilus or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said TTC1550, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said TTC1550, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said TTC1550, or a functional equivalent or a homolog thereof as depicted in column 8 of Table II, application no. 3, preferably a homolog or functional equivalent as depicted in column 8 of Table II B, application no. 3, and being depicted in the same respective line as said TTC1550, and preferably the activity is increased non-targeted.

Accordingly, in one embodiment, the molecule which activity is to be increased in the process of the invention is the gene product with an activity as a “homocitrate synthase”, preferably it is the molecule of section (a) or (b) of this paragraph.

In particular, it was observed that in plants, especially in Arabidopsis thaliana, increasing or generating the activity of a gene product non-targeted with the activity of a “homocitrate synthase”, preferably being encoded by a gene comprising the nucleic acid sequence SEQ ID NO. 12974, preferably the coding region thereof, conferred the production of or the increase in tyrosine compared with the wild type control.

The nucleic acid sequence of TTC1550 from Thermus thermophilus, e.g. as shown in the respective line in column 5 of Table I, application no. 3, has been published in E. And the activity of the gene product thereof is the activity of homocitrate synthase.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “homocitrate synthase”, especially from Thermus thermophilus or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said TTC1550, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said TTC1550, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said TTC1550, or a functional equivalent or a homolog thereof as depicted in column 8 of Table II, application no. 3, preferably a homolog or functional equivalent as depicted in column 8 of Table II B, application no. 3, and being depicted in the same respective line as said TTC1550, and preferably the activity is increased non-targeted.

Accordingly, in one embodiment, the molecule which activity is to be increased in the process of the invention is the gene product with an activity as a “homocitrate synthase”, preferably it is the molecule of section (a) or (b) of this paragraph.

In particular, it was observed that in plants, especially in Arabidopsis thaliana, increasing or generating the activity of a gene product non-targeted with the activity of a “homocitrate synthase”, preferably being encoded by a gene comprising the nucleic acid sequence SEQ ID NO. 12974, preferably the coding region thereof, conferred the production of or the increase in 5-oxoproline compared with the wild type control.

The nucleic acid sequence of TTC1918 from Thermus thermophilus, e.g. as shown in the respective line in column 5 of Table I, application no. 3, has been published in E. And the activity of the gene product thereof is the activity of oxidoreductase subunit.

Accordingly, in one embodiment, the process of the present invention for producing aspartate in a non-human organism, like a microorganism or a plant or a part thereof, comprises increasing or generating the activity of a gene product with the activity of a gene product conferring the activity of “oxidoreductase subunit”, especially from Thermus thermophilus or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said TTC1918, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said TTC1918, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said TTC1918, or a functional equivalent or a homolog thereof as depicted in column 8 of Table II, application no. 3, preferably a homolog or functional equivalent as depicted in column 8 of Table II B, application no. 3, and being depicted in the same respective line as said TTC1918, and preferably the activity is increased non-targeted.

Accordingly, in one embodiment, the molecule which activity is to be increased in the process of the invention is the gene product with an activity as a “oxidoreductase subunit”, preferably it is the molecule of section (a) or (b) of this paragraph.

In particular, it was observed that in plants, especially in Arabidopsis thaliana, increasing or generating the activity of a gene product non-targeted with the activity of a “oxidoreductase subunit”, preferably being encoded by a gene comprising the nucleic acid sequence SEQ ID NO. 62160, preferably the coding region thereof, conferred the production of or the increase in aspartate compared with the wild type control.

The nucleic acid sequence of TTC1918 from Thermus thermophilus, e.g. as shown in the respective line in column 5 of Table I, application no. 3, has been published in E. And the activity of the gene product thereof is the activity of oxidoreductase subunit.

Accordingly, in one embodiment, the process of the present invention for producing serine in a non-human organism, like a microorganism or a plant or a part thereof, comprises increasing or generating the activity of a gene product with the activity of a gene product conferring the activity of “oxidoreductase subunit”, especially from Thermus thermophilus or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said TTC1918, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said TTC1918, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said TTC1918, or a functional equivalent or a homolog thereof as depicted in column 8 of Table II, application no. 3, preferably a homolog or functional equivalent as depicted in column 8 of Table II B, application no. 3, and being depicted in the same respective line as said TTC1918, and preferably the activity is increased non-targeted.

Accordingly, in one embodiment, the molecule which activity is to be increased in the process of the invention is the gene product with an activity as a “oxidoreductase subunit”, preferably it is the molecule of section (a) or (b) of this paragraph.

In particular, it was observed that in plants, especially in Arabidopsis thaliana, increasing or generating the activity of a gene product non-targeted with the activity of a “oxidoreductase subunit”, preferably being encoded by a gene comprising the nucleic acid sequence SEQ ID NO. 62160, preferably the coding region thereof, conferred the production of or the increase in serine compared with the wild type control.

The nucleic acid sequence of XM_{—}473199 from Oryza sativa, e.g. as shown in the respective line in column 5 of Table I, application no. 3, has been published in G. And the activity of the gene product thereof is the activity of XM_{—}473199-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “XM_{—}473199-protein”, especially from Oryza sativa or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said XM_{—}473199, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said XM_{—}473199, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said XM_{—}473199, or a functional equivalent or a homolog thereof as depicted in column 8 of Table II, application no. 3, preferably a homolog or functional equivalent as depicted in column 8 of Table II B, application no. 3, and being depicted in the same respective line as said XM_{—}473199, and preferably the activity is increased non-targeted.

Accordingly, in one embodiment, the molecule which activity is to be increased in the process of the invention is the gene product with an activity as a “XM_{—}473199-protein”, preferably it is the molecule of section (a) or (b) of this paragraph.

In particular, it was observed that in plants, especially in Arabidopsis thaliana, increasing or generating the activity of a gene product non-targeted with the activity of a “XM_{—}473199-protein”, preferably being encoded by a gene comprising the nucleic acid sequence SEQ ID NO. 62244, preferably the coding region thereof, conferred the production of or the increase in aspartate compared with the wild type control.

The nucleic acid sequence of XM_{—}473199 from Oryza sativa, e.g. as shown in the respective line in column 5 of Table I, application no. 3, has been published in G. And the activity of the gene product thereof is the activity of XM_{—}473199-protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “XM_{—}473199-protein”, especially from Oryza sativa or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said XM_{—}473199, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said XM_{—}473199, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said XM_{—}473199, or a functional equivalent or a homolog thereof as depicted in column 8 of Table II, application no. 3, preferably a homolog or functional equivalent as depicted in column 8 of Table II B, application no. 3, and being depicted in the same respective line as said XM_{—}473199, and preferably the activity is increased non-targeted.

Accordingly, in one embodiment, the molecule which activity is to be increased in the process of the invention is the gene product with an activity as a “XM_{—}473199-protein”, preferably it is the molecule of section (a) or (b) of this paragraph.

In particular, it was observed that in plants, especially in Arabidopsis thaliana, increasing or generating the activity of a gene product non-targeted with the activity of a “XM_{—}473199-protein”, preferably being encoded by a gene comprising the nucleic acid sequence SEQ ID NO. 62244, preferably the coding region thereof, conferred the production of or the increase in phenylalanine compared with the wild type control.

The nucleic acid sequence of XM_{—}473199 from Oryza sativa, e.g. as shown in the respective line in column 5 of Table I, application no. 3, has been published in G. And the activity of the gene product thereof is the activity of XM_{—}473199-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “XM_{—}473199-protein”, especially from Oryza sativa or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said XM_{—}473199, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said XM_{—}473199, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said XM_{—}473199, or a functional equivalent or a homolog thereof as depicted in column 8 of Table II, application no. 3, preferably a homolog or functional equivalent as depicted in column 8 of Table II B, application no. 3, and being depicted in the same respective line as said XM_{—}473199, and preferably the activity is increased non-targeted.

Accordingly, in one embodiment, the molecule which activity is to be increased in the process of the invention is the gene product with an activity as a “XM_{—}473199-protein”, preferably it is the molecule of section (a) or (b) of this paragraph.

In particular, it was observed that in plants, especially in Arabidopsis thaliana, increasing or generating the activity of a gene product non-targeted with the activity of a “XM_{—}473199-protein”, preferably being encoded by a gene comprising the nucleic acid sequence SEQ ID NO. 62244, preferably the coding region thereof, conferred the production of or the increase in tyrosine compared with the wild type control.

The nucleic acid sequence of XM_{—}473199 from Oryza sativa, e.g. as shown in the respective line in column 5 of Table I, application no. 3, has been published in G. And the activity of the gene product thereof is the activity of XM_{—}473199-protein.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “XM_{—}473199-protein”, especially from Oryza sativa or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said XM_{—}473199, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said XM_{—}473199, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said XM_{—}473199, or a functional equivalent or a homolog thereof as depicted in column 8 of Table II, application no. 3, preferably a homolog or functional equivalent as depicted in column 8 of Table II B, application no. 3, and being depicted in the same respective line as said XM_{—}473199, and preferably the activity is increased non-targeted.

Accordingly, in one embodiment, the molecule which activity is to be increased in the process of the invention is the gene product with an activity as a “XM_{—}473199-protein”, preferably it is the molecule of section (a) or (b) of this paragraph.

In particular, it was observed that in plants, especially in Arabidopsis thaliana, increasing or generating the activity of a gene product non-targeted with the activity of a “XM_{—}473199-protein”, preferably being encoded by a gene comprising the nucleic acid sequence SEQ ID NO. 62244, preferably the coding region thereof, conferred the production of or the increase in 5-oxoproline compared with the wild type control.

The nucleic acid sequence of Ybl021c from Saccharomyces cerevisiae, e.g. as shown in the respective line in column 5 of Table I, application no. 3, has been published in A. And the activity of the gene product thereof is the activity of transcription factor.

Accordingly, in one embodiment, the process of the present invention for producing glycine in a non-human organism, like a microorganism or a plant or a part thereof, comprises increasing or generating the activity of a gene product with the activity of a gene product conferring the activity of “transcription factor”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ybl021c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ybl021c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ybl021c, or a functional equivalent or a homolog thereof as depicted in column 8 of Table II, application no. 3, preferably a homolog or functional equivalent as depicted in column 8 of Table II B, application no. 3, and being depicted in the same respective line as said Ybl021c, and preferably the activity is increased non-targeted.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 62524, preferably the coding region thereof, conferred the production of or the increase in glycine compared with the wild type control.

The nucleic acid sequence of Ybl021 c from Saccharomyces cerevisiae, e.g. as shown in the respective line in column 5 of Table I, application no. 3, has been published in A. And the activity of the gene product thereof is the activity of transcription factor.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “transcription factor”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ybl021c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ybl021c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ybl021c, or a functional equivalent or a homolog thereof as depicted in column 8 of Table II, application no. 3, preferably a homolog or functional equivalent as depicted in column 8 of Table II B, application no. 3, and being depicted in the same respective line as said Ybl021c, and preferably the activity is increased non-targeted.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 62524, preferably the coding region thereof, conferred the production of or the increase in phenylalanine compared with the wild type control.

The nucleic acid sequence of Ybr160w from Saccharomyces cerevisiae, e.g. as shown in the respective line in column 5 of Table I, application no. 3, has been published in A. And the activity of the gene product thereof is the activity of cell division control protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “cell division control protein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ybr160w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ybr160w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ybr160w, or a functional equivalent or a homolog thereof as depicted in column 8 of Table II, application no. 3, preferably a homolog or functional equivalent as depicted in column 8 of Table II B, application no. 3, and being depicted in the same respective line as said Ybr160w, and preferably the activity is increased non-targeted.

Accordingly, in one embodiment, the molecule which activity is to be increased in the process of the invention is the gene product with an activity as a “cell division control protein”, preferably it is the molecule of section (a) or (b) of this paragraph.

In particular, it was observed that in plants, especially in Arabidopsis thaliana, increasing or generating the activity of a gene product non-targeted with the activity of a “cell division control protein”, preferably being encoded by a gene comprising the nucleic acid sequence SEQ ID NO. 62717, preferably the coding region thereof, conferred the production of or the increase in aspartate compared with the wild type control.

The nucleic acid sequence of Ybr160w from Saccharomyces cerevisiae, e.g. as shown in the respective line in column 5 of Table I, application no. 3, has been published in A. And the activity of the gene product thereof is the activity of cell division control protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “cell division control protein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ybr160w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ybr160w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ybr160w, or a functional equivalent or a homolog thereof as depicted in column 8 of Table II, application no. 3, preferably a homolog or functional equivalent as depicted in column 8 of Table II B, application no. 3, and being depicted in the same respective line as said Ybr160w, and preferably the activity is increased non-targeted.

Accordingly, in one embodiment, the molecule which activity is to be increased in the process of the invention is the gene product with an activity as a “cell division control protein”, preferably it is the molecule of section (a) or (b) of this paragraph.

In particular, it was observed that in plants, especially in Arabidopsis thaliana, increasing or generating the activity of a gene product non-targeted with the activity of a “cell division control protein”, preferably being encoded by a gene comprising the nucleic acid sequence SEQ ID NO. 62717, preferably the coding region thereof, conferred the production of or the increase in tyrosine compared with the wild type control.

The nucleic acid sequence of Ybr249c from Saccharomyces cerevisiae, e.g. as shown in the respective line in column 5 of Table I, application no. 3, has been published in A. And the activity of the gene product thereof is the activity of 3-deoxy-7-phosphoheptulonate synthase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “3-deoxy-7-phosphoheptulonate synthase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ybr249c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ybr249c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ybr249c, or a functional equivalent or a homolog thereof as depicted in column 8 of Table II, application no. 3, preferably a homolog or functional equivalent as depicted in column 8 of Table II B, application no. 3, and being depicted in the same respective line as said Ybr249c, and preferably the activity is increased plastidic.

Accordingly, in one embodiment, the molecule which activity is to be increased in the process of the invention is the gene product with an activity as a “3-deoxy-7-phosphoheptulonate synthase”, preferably it is the molecule of section (a) or (b) of this paragraph.

In particular, it was observed that in plants, especially in Arabidopsis thaliana, increasing or generating the activity of a gene product plastidic with the activity of a “3-deoxy-7-phosphoheptulonate synthase”, preferably being encoded by a gene comprising the nucleic acid sequence SEQ ID NO. 88879, preferably the coding region thereof, conferred the production of or the increase in phenylalanine compared with the wild type control.

The nucleic acid sequence of Ybr249c from Saccharomyces cerevisiae, e.g. as shown in the respective line in column 5 of Table I, application no. 3, has been published in A. And the activity of the gene product thereof is the activity of 3-deoxy-7-phosphoheptulonate synthase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “3-deoxy-7-phosphoheptulonate synthase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ybr249c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ybr249c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ybr249c, or a functional equivalent or a homolog thereof as depicted in column 8 of Table II, application no. 3, preferably a homolog or functional equivalent as depicted in column 8 of Table II B, application no. 3, and being depicted in the same respective line as said Ybr249c, and preferably the activity is increased plastidic.

Accordingly, in one embodiment, the molecule which activity is to be increased in the process of the invention is the gene product with an activity as a “3-deoxy-7-phosphoheptulonate synthase”, preferably it is the molecule of section (a) or (b) of this paragraph.

In particular, it was observed that in plants, especially in Arabidopsis thaliana, increasing or generating the activity of a gene product plastidic with the activity of a “3-deoxy-7-phosphoheptulonate synthase”, preferably being encoded by a gene comprising the nucleic acid sequence SEQ ID NO. 88879, preferably the coding region thereof, conferred the production of or the increase in tyrosine compared with the wild type control.

The nucleic acid sequence of Ybr296c from Saccharomyces cerevisiae, e.g. as shown in the respective line in column 5 of Table I, application no. 3, has been published in A. And the activity of the gene product thereof is the activity of phosphate permease.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “phosphate permease”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ybr296c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ybr296c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ybr296c, or a functional equivalent or a homolog thereof as depicted in column 8 of Table II, application no. 3, preferably a homolog or functional equivalent as depicted in column 8 of Table II B, application no. 3, and being depicted in the same respective line as said Ybr296c, and preferably the activity is increased non-targeted.

Accordingly, in one embodiment, the molecule which activity is to be increased in the process of the invention is the gene product with an activity as a “phosphate permease”, preferably it is the molecule of section (a) or (b) of this paragraph.

In particular, it was observed that in plants, especially in Arabidopsis thaliana, increasing or generating the activity of a gene product non-targeted with the activity of a “phosphate permease”, preferably being encoded by a gene comprising the nucleic acid sequence SEQ ID NO. 89317, preferably the coding region thereof, conferred the production of or the increase in 5-oxoproline compared with the wild type control.

The nucleic acid sequence of Ycl064c from Saccharomyces cerevisiae, e.g. as shown in the respective line in column 5 of Table I, application no. 3, has been published in A. And the activity of the gene product thereof is the activity of serine/threonine dehydratase.

Accordingly, in one embodiment, the process of the present invention for producing alanine in a non-human organism, like a microorganism or a plant or a part thereof, comprises increasing or generating the activity of a gene product with the activity of a gene product conferring the activity of “serine/threonine dehydratase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ycl064c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ycl064c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ycl064c, or a functional equivalent or a homolog thereof as depicted in column 8 of Table II, application no. 3, preferably a homolog or functional equivalent as depicted in column 8 of Table II B, application no. 3, and being depicted in the same respective line as said Ycl064c, and preferably the activity is increased plastidic.

Accordingly, in one embodiment, the molecule which activity is to be increased in the process of the invention is the gene product with an activity as a “serine/threonine dehydratase”, preferably it is the molecule of section (a) or (b) of this paragraph.

In particular, it was observed that in plants, especially in Arabidopsis thaliana, increasing or generating the activity of a gene product plastidic with the activity of a “serine/threonine dehydratase”, preferably being encoded by a gene comprising the nucleic acid sequence SEQ ID NO. 89395, preferably the coding region thereof, conferred the production of or the increase in alanine compared with the wild type control.

The nucleic acid sequence of Ycl064c from Saccharomyces cerevisiae, e.g. as shown in the respective line in column 5 of Table I, application no. 3, has been published in A. And the activity of the gene product thereof is the activity of serine/threonine dehydratase.

Accordingly, in one embodiment, the process of the present invention for producing glycine in a non-human organism, like a microorganism or a plant or a part thereof, comprises increasing or generating the activity of a gene product with the activity of a gene product conferring the activity of “serine/threonine dehydratase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ycl064c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ycl064c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ycl064c, or a functional equivalent or a homolog thereof as depicted in column 8 of Table II, application no. 3, preferably a homolog or functional equivalent as depicted in column 8 of Table II B, application no. 3, and being depicted in the same respective line as said Ycl064c, and preferably the activity is increased plastidic.

Accordingly, in one embodiment, the molecule which activity is to be increased in the process of the invention is the gene product with an activity as a “serine/threonine dehydratase”, preferably it is the molecule of section (a) or (b) of this paragraph.

In particular, it was observed that in plants, especially in Arabidopsis thaliana, increasing or generating the activity of a gene product plastidic with the activity of a “serine/threonine dehydratase”, preferably being encoded by a gene comprising the nucleic acid sequence SEQ ID NO. 89395, preferably the coding region thereof, conferred the production of or the increase in glycine compared with the wild type control.

The nucleic acid sequence of Ydl159w from Saccharomyces cerevisiae, e.g. as shown in the respective line in column 5 of Table I, application no. 3, has been published in A. And the activity of the gene product thereof is the activity of protein kinase.

Accordingly, in one embodiment, the process of the present invention for producing aspartate in a non-human organism, like a microorganism or a plant or a part thereof, comprises increasing or generating the activity of a gene product with the activity of a gene product conferring the activity of “protein kinase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ydl159w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ydl159w, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ydl159w, or a functional equivalent or a homolog thereof as depicted in column 8 of Table II, application no. 3, preferably a homolog or functional equivalent as depicted in column 8 of Table II B, application no. 3, and being depicted in the same respective line as said Ydl159w, and preferably the activity is increased plastidic.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product plastidic with the activity of a “protein kinase”, preferably being encoded by a gene comprising the nucleic acid sequence SEQ ID NO. 14275, preferably the coding region thereof, conferred the production of or the increase in aspartate compared with the wild type control.

The nucleic acid sequence of Ydl188c from Saccharomyces cerevisiae, e.g. as shown in the respective line in column 5 of Table I, application no. 3, has been published in A. And the activity of the gene product thereof is the activity of Serine/threonine-protein phosphatase.

Accordingly, in one embodiment, the process of the present invention for producing homoserine in a non-human organism, like a microorganism or a plant or a part thereof, comprises increasing or generating the activity of a gene product with the activity of a gene product conferring the activity of “Serine/threonine-protein phosphatase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ydl188c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ydl188c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ydl188c, or a functional equivalent or a homolog thereof as depicted in column 8 of Table II, application no. 3, preferably a homolog or functional equivalent as depicted in column 8 of Table II B, application no. 3, and being depicted in the same respective line as said Ydl188c, and preferably the activity is increased plastidic.

Accordingly, in one embodiment, the molecule which activity is to be increased in the process of the invention is the gene product with an activity as a “Serine/threonine-protein phosphatase”, preferably it is the molecule of section (a) or (b) of this paragraph.

In particular, it was observed that in plants, especially in Arabidopsis thaliana, increasing or generating the activity of a gene product plastidic with the activity of a “Serine/threonine-protein phosphatase”, preferably being encoded by a gene comprising the nucleic acid sequence SEQ ID NO. 89483, preferably the coding region thereof, conferred the production of or the increase in homoserine compared with the wild type control.

The nucleic acid sequence of Ydl188c from Saccharomyces cerevisiae, e.g. as shown in the respective line in column 5 of Table I, application no. 3, has been published in A. And the activity of the gene product thereof is the activity of Serine/threonine-protein phosphatase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “Serine/threonine-protein phosphatase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ydl188c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ydl188c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ydl188c, or a functional equivalent or a homolog thereof as depicted in column 8 of Table II, application no. 3, preferably a homolog or functional equivalent as depicted in column 8 of Table II B, application no. 3, and being depicted in the same respective line as said Ydl188c, and preferably the activity is increased plastidic.

Accordingly, in one embodiment, the molecule which activity is to be increased in the process of the invention is the gene product with an activity as a “Serine/threonine-protein phosphatase”, preferably it is the molecule of section (a) or (b) of this paragraph.

In particular, it was observed that in plants, especially in Arabidopsis thaliana, increasing or generating the activity of a gene product plastidic with the activity of a “Serine/threonine-protein phosphatase”, preferably being encoded by a gene comprising the nucleic acid sequence SEQ ID NO. 89483, preferably the coding region thereof, conferred the production of or the increase in phenylalanine compared with the wild type control.

The nucleic acid sequence of Ydl209c from Saccharomyces cerevisiae, e.g. as shown in the respective line in column 5 of Table I, application no. 3, has been published in A. And the activity of the gene product thereof is the activity of Pre-mRNA-splicing factor.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “Pre-mRNA-splicing factor”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ydl209c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ydl209c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ydl209c, or a functional equivalent or a homolog thereof as depicted in column 8 of Table II, application no. 3, preferably a homolog or functional equivalent as depicted in column 8 of Table II B, application no. 3, and being depicted in the same respective line as said Ydl209c, and preferably the activity is increased plastidic.

Accordingly, in one embodiment, the molecule which activity is to be increased in the process of the invention is the gene product with an activity as a “Pre-mRNA-splicing factor”, preferably it is the molecule of section (a) or (b) of this paragraph.

In particular, it was observed that in plants, especially in Arabidopsis thaliana, increasing or generating the activity of a gene product plastidic with the activity of a “Pre-mRNA-splicing factor”, preferably being encoded by a gene comprising the nucleic acid sequence SEQ ID NO. 89936, preferably the coding region thereof, conferred the production of or the increase in 5-oxoproline compared with the wild type control.

The nucleic acid sequence of Ydr007w from Saccharomyces cerevisiae, e.g. as shown in the respective line in column 5 of Table I, application no. 3, has been published in A. And the activity of the gene product thereof is the activity of N-(5′-phosphoribosyl)anthranilate isomerase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “N-(5′-phosphoribosyl)anthranilate isomerase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ydr007w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ydr007w, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ydr007w, or a functional equivalent or a homolog thereof as depicted in column 8 of Table II, application no. 3, preferably a homolog or functional equivalent as depicted in column 8 of Table II B, application no. 3, and being depicted in the same respective line as said Ydr007w, and preferably the activity is increased plastidic.

Accordingly, in one embodiment, the molecule which activity is to be increased in the process of the invention is the gene product with an activity as a “N-(5′-phosphoribosyl)anthranilate isomerase”, preferably it is the molecule of section (a) or (b) of this paragraph.

In particular, it was observed that in plants, especially in Arabidopsis thaliana, increasing or generating the activity of a gene product plastidic with the activity of a “N-(5′-phosphoribosyl)anthranilate isomerase”, preferably being encoded by a gene comprising the nucleic acid sequence SEQ ID NO. 89973, preferably the coding region thereof, conferred the production of or the increase in phenylalanine compared with the wild type control.

The nucleic acid sequence of Ydr046c from Saccharomyces cerevisiae, e.g. as shown in the respective line in column 5 of Table I, application no. 3, has been published in A. And the activity of the gene product thereof is the activity of branched-chain amino acid permease.

Accordingly, in one embodiment, the process of the present invention for producing tyrosine in a non-human organism, like a microorganism or a plant or a part thereof, comprises increasing or generating the activity of a gene product with the activity of a gene product conferring the activity of “branched-chain amino acid permease”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ydr046c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ydr046c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ydr046c, or a functional equivalent or a homolog thereof as depicted in column 8 of Table II, application no. 3, preferably a homolog or functional equivalent as depicted in column 8 of Table II B, application no. 3, and being depicted in the same respective line as said Ydr046c, and preferably the activity is increased plastidic.

Accordingly, in one embodiment, the molecule which activity is to be increased in the process of the invention is the gene product with an activity as a “branched-chain amino acid permease”, preferably it is the molecule of section (a) or (b) of this paragraph.

In particular, it was observed that in plants, especially in Arabidopsis thaliana, increasing or generating the activity of a gene product plastidic with the activity of a “branched-chain amino acid permease”, preferably being encoded by a gene comprising the nucleic acid sequence SEQ ID NO. 63544, preferably the coding region thereof, conferred the production of or the increase in tyrosine compared with the wild type control.

The nucleic acid sequence of Ydr183w from Saccharomyces cerevisiae, e.g. as shown in the respective line in column 5 of Table I, application no. 3, has been published in A. And the activity of the gene product thereof is the activity of ydr183w-protein.

Accordingly, in one embodiment, the process of the present invention for producing serine in a non-human organism, like a microorganism or a plant or a part thereof, comprises increasing or generating the activity of a gene product with the activity of a gene product conferring the activity of “ydr183w-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ydr183w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ydr183w, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ydr183w, or a functional equivalent or a homolog thereof as depicted in column 8 of Table II, application no. 3, preferably a homolog or functional equivalent as depicted in column 8 of Table II B, application no. 3, and being depicted in the same respective line as said Ydr183w, and preferably the activity is increased plastidic.

Accordingly, in one embodiment, the molecule which activity is to be increased in the process of the invention is the gene product with an activity as a “ydr183w-protein”, preferably it is the molecule of section (a) or (b) of this paragraph.

In particular, it was observed that in plants, especially in Arabidopsis thaliana, increasing or generating the activity of a gene product plastidic with the activity of a “ydr183w-protein”, preferably being encoded by a gene comprising the nucleic acid sequence SEQ ID NO. 90103, preferably the coding region thereof, conferred the production of or the increase in serine compared with the wild type control.

The nucleic acid sequence of Ydr273w from Saccharomyces cerevisiae, e.g. as shown in the respective line in column 5 of Table I, application no. 3, has been published in A. And the activity of the gene product thereof is the activity of ydr273w-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “ydr273w-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ydr273w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ydr273w, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ydr273w, or a functional equivalent or a homolog thereof as depicted in column 8 of Table II, application no. 3, preferably a homolog or functional equivalent as depicted in column 8 of Table II B, application no. 3, and being depicted in the same respective line as said Ydr273w, and preferably the activity is increased plastidic.

Accordingly, in one embodiment, the molecule which activity is to be increased in the process of the invention is the gene product with an activity as a “ydr273w-protein”, preferably it is the molecule of section (a) or (b) of this paragraph.

In particular, it was observed that in plants, especially in Arabidopsis thaliana, increasing or generating the activity of a gene product plastidic with the activity of a “ydr273w-protein”, preferably being encoded by a gene comprising the nucleic acid sequence SEQ ID NO. 90159, preferably the coding region thereof, conferred the production of or the increase in aspartate compared with the wild type control.

The nucleic acid sequence of Ydr273w from Saccharomyces cerevisiae, e.g. as shown in the respective line in column 5 of Table I, application no. 3, has been published in A. And the activity of the gene product thereof is the activity of ydr273w-protein.

Accordingly, in one embodiment, the process of the present invention for producing citrulline in a non-human organism, like a microorganism or a plant or a part thereof, comprises increasing or generating the activity of a gene product with the activity of a gene product conferring the activity of “ydr273w-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ydr273w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ydr273w, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ydr273w, or a functional equivalent or a homolog thereof as depicted in column 8 of Table II, application no. 3, preferably a homolog or functional equivalent as depicted in column 8 of Table II B, application no. 3, and being depicted in the same respective line as said Ydr273w, and preferably the activity is increased plastidic.

Accordingly, in one embodiment, the molecule which activity is to be increased in the process of the invention is the gene product with an activity as a “ydr273w-protein”, preferably it is the molecule of section (a) or (b) of this paragraph.

In particular, it was observed that in plants, especially in Arabidopsis thaliana, increasing or generating the activity of a gene product plastidic with the activity of a “ydr273w-protein”, preferably being encoded by a gene comprising the nucleic acid sequence SEQ ID NO. 90159, preferably the coding region thereof, conferred the production of or the increase in citrulline compared with the wild type control.

The nucleic acid sequence of Ydr507c from Saccharomyces cerevisiae, e.g. as shown in the respective line in column 5 of Table I, application no. 3, has been published in A. And the activity of the gene product thereof is the activity of ydr507c-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “ydr507c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ydr507c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ydr507c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ydr507c, or a functional equivalent or a homolog thereof as depicted in column 8 of Table II, application no. 3, preferably a homolog or functional equivalent as depicted in column 8 of Table II B, application no. 3, and being depicted in the same respective line as said Ydr507c, and preferably the activity is increased non-targeted.

Accordingly, in one embodiment, the molecule which activity is to be increased in the process of the invention is the gene product with an activity as a “ydr507c-protein”, preferably it is the molecule of section (a) or (b) of this paragraph.

In particular, it was observed that in plants, especially in Arabidopsis thaliana, increasing or generating the activity of a gene product non-targeted with the activity of a “ydr507c-protein”, preferably being encoded by a gene comprising the nucleic acid sequence SEQ ID NO. 90165, preferably the coding region thereof, conferred the production of or the increase in aspartate compared with the wild type control.

The nucleic acid sequence of Yer014w from Saccharomyces cerevisiae, e.g. as shown in the respective line in column 5 of Table I, application no. 3, has been published in A. And the activity of the gene product thereof is the activity of yer014w-protein.

Accordingly, in one embodiment, the process of the present invention for producing asparagine in a non-human organism, like a microorganism or a plant or a part thereof, comprises increasing or generating the activity of a gene product with the activity of a gene product conferring the activity of “yer014w-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yer014w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yer014w, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yer014w, or a functional equivalent or a homolog thereof as depicted in column 8 of Table II, application no. 3, preferably a homolog or functional equivalent as depicted in column 8 of Table II B, application no. 3, and being depicted in the same respective line as said Yer014w, and preferably the activity is increased plastidic.

Accordingly, in one embodiment, the molecule which activity is to be increased in the process of the invention is the gene product with an activity as a “yer014w-protein”, preferably it is the molecule of section (a) or (b) of this paragraph.

In particular, it was observed that in plants, especially in Arabidopsis thaliana, increasing or generating the activity of a gene product plastidic with the activity of a “yer014w-protein”, preferably being encoded by a gene comprising the nucleic acid sequence SEQ ID NO. 63745, preferably the coding region thereof, conferred the production of or the increase in asparagine compared with the wild type control.

The nucleic acid sequence of Yer014w from Saccharomyces cerevisiae, e.g. as shown in the respective line in column 5 of Table I, application no. 3, has been published in A. And the activity of the gene product thereof is the activity of yer014w-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “yer014w-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yer014w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yer014w, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yer014w, or a functional equivalent or a homolog thereof as depicted in column 8 of Table II, application no. 3, preferably a homolog or functional equivalent as depicted in column 8 of Table II B, application no. 3, and being depicted in the same respective line as said Yer014w, and preferably the activity is increased plastidic.

Accordingly, in one embodiment, the molecule which activity is to be increased in the process of the invention is the gene product with an activity as a “yer014w-protein”, preferably it is the molecule of section (a) or (b) of this paragraph.

In particular, it was observed that in plants, especially in Arabidopsis thaliana, increasing or generating the activity of a gene product plastidic with the activity of a “yer014w-protein”, preferably being encoded by a gene comprising the nucleic acid sequence SEQ ID NO. 63745, preferably the coding region thereof, conferred the production of or the increase in aspartate compared with the wild type control.

The nucleic acid sequence of Yer014w from Saccharomyces cerevisiae, e.g. as shown in the respective line in column 5 of Table I, application no. 3, has been published in A. And the activity of the gene product thereof is the activity of yer014w-protein.

Accordingly, in one embodiment, the process of the present invention for producing serine in a non-human organism, like a microorganism or a plant or a part thereof, comprises increasing or generating the activity of a gene product with the activity of a gene product conferring the activity of “yer014w-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yer014w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yer014w, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yer014w, or a functional equivalent or a homolog thereof as depicted in column 8 of Table II, application no. 3, preferably a homolog or functional equivalent as depicted in column 8 of Table II B, application no. 3, and being depicted in the same respective line as said Yer014w, and preferably the activity is increased plastidic.

Accordingly, in one embodiment, the molecule which activity is to be increased in the process of the invention is the gene product with an activity as a “yer014w-protein”, preferably it is the molecule of section (a) or (b) of this paragraph.

In particular, it was observed that in plants, especially in Arabidopsis thaliana, increasing or generating the activity of a gene product plastidic with the activity of a “yer014w-protein”, preferably being encoded by a gene comprising the nucleic acid sequence SEQ ID NO. 63745, preferably the coding region thereof, conferred the production of or the increase in serine compared with the wild type control.

The nucleic acid sequence of Yer106w from Saccharomyces cerevisiae, e.g. as shown in the respective line in column 5 of Table I, application no. 3, has been published in A. And the activity of the gene product thereof is the activity of yer106w-protein.

Accordingly, in one embodiment, the process of the present invention for producing citrulline in a non-human organism, like a microorganism or a plant or a part thereof, comprises increasing or generating the activity of a gene product with the activity of a gene product conferring the activity of “yer106w-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yer106w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yer106w, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yer106w, or a functional equivalent or a homolog thereof as depicted in column 8 of Table II, application no. 3, preferably a homolog or functional equivalent as depicted in column 8 of Table II B, application no. 3, and being depicted in the same respective line as said Yer106w, and preferably the activity is increased plastidic.

Accordingly, in one embodiment, the molecule which activity is to be increased in the process of the invention is the gene product with an activity as a “yer106w-protein”, preferably it is the molecule of section (a) or (b) of this paragraph.

In particular, it was observed that in plants, especially in Arabidopsis thaliana, increasing or generating the activity of a gene product plastidic with the activity of a “yer106w-protein”, preferably being encoded by a gene comprising the nucleic acid sequence SEQ ID NO. 63803, preferably the coding region thereof, conferred the production of or the increase in citrulline compared with the wild type control.

The nucleic acid sequence of Yfl016c from Saccharomyces cerevisiae, e.g. as shown in the respective line in column 5 of Table I, application no. 3, has been published in A. And the activity of the gene product thereof is the activity of molecular chaperone portein.

Accordingly, in one embodiment, the process of the present invention for producing serine in a non-human organism, like a microorganism or a plant or a part thereof, comprises increasing or generating the activity of a gene product with the activity of a gene product conferring the activity of “molecular chaperone portein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yfl016c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yfl016c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yfl016c, or a functional equivalent or a homolog thereof as depicted in column 8 of Table II, application no. 3, preferably a homolog or functional equivalent as depicted in column 8 of Table II B, application no. 3, and being depicted in the same respective line as said Yfl016c, and preferably the activity is increased non-targeted.

Accordingly, in one embodiment, the molecule which activity is to be increased in the process of the invention is the gene product with an activity as a “molecular chaperone portein”, preferably it is the molecule of section (a) or (b) of this paragraph.

In particular, it was observed that in plants, especially in Arabidopsis thaliana, increasing or generating the activity of a gene product non-targeted with the activity of a “molecular chaperone portein”, preferably being encoded by a gene comprising the nucleic acid sequence SEQ ID NO. 63807, preferably the coding region thereof, conferred the production of or the increase in serine compared with the wild type control.

The nucleic acid sequence of Yfl016c from Saccharomyces cerevisiae, e.g. as shown in the respective line in column 5 of Table I, application no. 3, has been published in A. And the activity of the gene product thereof is the activity of molecular chaperone portein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “molecular chaperone portein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yfl016c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yfl016c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yfl016c, or a functional equivalent or a homolog thereof as depicted in column 8 of Table II, application no. 3, preferably a homolog or functional equivalent as depicted in column 8 of Table II B, application no. 3, and being depicted in the same respective line as said Yfl016c, and preferably the activity is increased non-targeted.

Accordingly, in one embodiment, the molecule which activity is to be increased in the process of the invention is the gene product with an activity as a “molecular chaperone portein”, preferably it is the molecule of section (a) or (b) of this paragraph.

In particular, it was observed that in plants, especially in Arabidopsis thaliana, increasing or generating the activity of a gene product non-targeted with the activity of a “molecular chaperone portein”, preferably being encoded by a gene comprising the nucleic acid sequence SEQ ID NO. 63807, preferably the coding region thereof, conferred the production of or the increase in tyrosine compared with the wild type control.

The nucleic acid sequence of Yfl019c from Saccharomyces cerevisiae, e.g. as shown in the respective line in column 5 of Table I, application no. 3, has been published in A. And the activity of the gene product thereof is the activity of YFL019C-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “YFL019C-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yfl019c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yfl019c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yfl019c, or a functional equivalent or a homolog thereof as depicted in column 8 of Table II, application no. 3, preferably a homolog or functional equivalent as depicted in column 8 of Table II B, application no. 3, and being depicted in the same respective line as said Yfl019c, and preferably the activity is increased non-targeted.

Accordingly, in one embodiment, the molecule which activity is to be increased in the process of the invention is the gene product with an activity as a “YFL019C-protein”, preferably it is the molecule of section (a) or (b) of this paragraph.

In particular, it was observed that in plants, especially in Arabidopsis thaliana, increasing or generating the activity of a gene product non-targeted with the activity of a “YFL019C-protein”, preferably being encoded by a gene comprising the nucleic acid sequence SEQ ID NO. 64144, preferably the coding region thereof, conferred the production of or the increase in aspartate compared with the wild type control.

The nucleic acid sequence of Yfl019c from Saccharomyces cerevisiae, e.g. as shown in the respective line in column 5 of Table I, application no. 3, has been published in A. And the activity of the gene product thereof is the activity of YFL019C-protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “YFL019C-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yfl019c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yfl019c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yfl019c, or a functional equivalent or a homolog thereof as depicted in column 8 of Table II, application no. 3, preferably a homolog or functional equivalent as depicted in column 8 of Table II B, application no. 3, and being depicted in the same respective line as said Yfl019c, and preferably the activity is increased non-targeted.

Accordingly, in one embodiment, the molecule which activity is to be increased in the process of the invention is the gene product with an activity as a “YFL019C-protein”, preferably it is the molecule of section (a) or (b) of this paragraph.

In particular, it was observed that in plants, especially in Arabidopsis thaliana, increasing or generating the activity of a gene product non-targeted with the activity of a “YFL019C-protein”, preferably being encoded by a gene comprising the nucleic acid sequence SEQ ID NO. 64144, preferably the coding region thereof, conferred the production of or the increase in phenylalanine compared with the wild type control.

The nucleic acid sequence of Yfl030w from Saccharomyces cerevisiae, e.g. as shown in the respective line in column 5 of Table I, application no. 3, has been published in A. And the activity of the gene product thereof is the activity of aminotransferase.

Accordingly, in one embodiment, the process of the present invention for producing alanine in a non-human organism, like a microorganism or a plant or a part thereof, comprises increasing or generating the activity of a gene product with the activity of a gene product conferring the activity of “aminotransferase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yfl030w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yfl030w, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yfl030w, or a functional equivalent or a homolog thereof as depicted in column 8 of Table II, application no. 3, preferably a homolog or functional equivalent as depicted in column 8 of Table II B, application no. 3, and being depicted in the same respective line as said Yfl030w, and preferably the activity is increased plastidic.

Accordingly, in one embodiment, the molecule which activity is to be increased in the process of the invention is the gene product with an activity as a “aminotransferase”, preferably it is the molecule of section (a) or (b) of this paragraph.

In particular, it was observed that in plants, especially in Arabidopsis thaliana, increasing or generating the activity of a gene product plastidic with the activity of a “aminotransferase”, preferably being encoded by a gene comprising the nucleic acid sequence SEQ ID NO. 90189, preferably the coding region thereof, conferred the production of or the increase in alanine compared with the wild type control.

The nucleic acid sequence of Yfl030w from Saccharomyces cerevisiae, e.g. as shown in the respective line in column 5 of Table I, application no. 3, has been published in A. And the activity of the gene product thereof is the activity of aminotransferase.

Accordingly, in one embodiment, the process of the present invention for producing glycine in a non-human organism, like a microorganism or a plant or a part thereof, comprises increasing or generating the activity of a gene product with the activity of a gene product conferring the activity of “aminotransferase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yfl030w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yfl030w, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yfl030w, or a functional equivalent or a homolog thereof as depicted in column 8 of Table II, application no. 3, preferably a homolog or functional equivalent as depicted in column 8 of Table II B, application no. 3, and being depicted in the same respective line as said Yfl030w, and preferably the activity is increased plastidic.

Accordingly, in one embodiment, the molecule which activity is to be increased in the process of the invention is the gene product with an activity as a “aminotransferase”, preferably it is the molecule of section (a) or (b) of this paragraph.

In particular, it was observed that in plants, especially in Arabidopsis thaliana, increasing or generating the activity of a gene product plastidic with the activity of a “aminotransferase”, preferably being encoded by a gene comprising the nucleic acid sequence SEQ ID NO. 90189, preferably the coding region thereof, conferred the production of or the increase in glycine compared with the wild type control.

The nucleic acid sequence of Yfl054c from Saccharomyces cerevisiae, e.g. as shown in the respective line in column 5 of Table I, application no. 3, has been published in A. And the activity of the gene product thereof is the activity of yfl054c-protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “yfl054c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yfl054c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yfl054c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yfl054c, or a functional equivalent or a homolog thereof as depicted in column 8 of Table II, application no. 3, preferably a homolog or functional equivalent as depicted in column 8 of Table II B, application no. 3, and being depicted in the same respective line as said Yfl054c, and preferably the activity is increased plastidic.

Accordingly, in one embodiment, the molecule which activity is to be increased in the process of the invention is the gene product with an activity as a “yfl054c-protein”, preferably it is the molecule of section (a) or (b) of this paragraph.

In particular, it was observed that in plants, especially in Arabidopsis thaliana, increasing or generating the activity of a gene product plastidic with the activity of a “yfl054c-protein”, preferably being encoded by a gene comprising the nucleic acid sequence SEQ ID NO. 64148, preferably the coding region thereof, conferred the production of or the increase in phenylalanine compared with the wild type control.

The nucleic acid sequence of Yfl054c from Saccharomyces cerevisiae, e.g. as shown in the respective line in column 5 of Table I, application no. 3, has been published in A. And the activity of the gene product thereof is the activity of yfl054c-protein.

Accordingly, in one embodiment, the process of the present invention for producing serine in a non-human organism, like a microorganism or a plant or a part thereof, comprises increasing or generating the activity of a gene product with the activity of a gene product conferring the activity of “yfl054c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yfl054c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yfl054c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yfl054c, or a functional equivalent or a homolog thereof as depicted in column 8 of Table II, application no. 3, preferably a homolog or functional equivalent as depicted in column 8 of Table II B, application no. 3, and being depicted in the same respective line as said Yfl054c, and preferably the activity is increased plastidic.

Accordingly, in one embodiment, the molecule which activity is to be increased in the process of the invention is the gene product with an activity as a “yfl054c-protein”, preferably it is the molecule of section (a) or (b) of this paragraph.

In particular, it was observed that in plants, especially in Arabidopsis thaliana, increasing or generating the activity of a gene product plastidic with the activity of a “yfl054c-protein”, preferably being encoded by a gene comprising the nucleic acid sequence SEQ ID NO. 64148, preferably the coding region thereof, conferred the production of or the increase in serine compared with the wild type control.

The nucleic acid sequence of Ygl096w from Saccharomyces cerevisiae, e.g. as shown in the respective line in column 5 of Table I, application no. 3, has been published in A. And the activity of the gene product thereof is the activity of ygl096w-protein.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “ygl096w-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ygl096w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ygl096w, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ygl096w, or a functional equivalent or a homolog thereof as depicted in column 8 of Table II, application no. 3, preferably a homolog or functional equivalent as depicted in column 8 of Table II B, application no. 3, and being depicted in the same respective line as said Ygl096w, and preferably the activity is increased plastidic.

Accordingly, in one embodiment, the molecule which activity is to be increased in the process of the invention is the gene product with an activity as a “ygl096w-protein”, preferably it is the molecule of section (a) or (b) of this paragraph.

In particular, it was observed that in plants, especially in Arabidopsis thaliana, increasing or generating the activity of a gene product plastidic with the activity of a “ygl096w-protein”, preferably being encoded by a gene comprising the nucleic acid sequence SEQ ID NO. 90278, preferably the coding region thereof, conferred the production of or the increase in 5-oxoproline compared with the wild type control.

The nucleic acid sequence of Ygl237c from Saccharomyces cerevisiae, e.g. as shown in the respective line in column 5 of Table I, application no. 3, has been published in A. And the activity of the gene product thereof is the activity of ygl237c-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “ygl237c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ygl237c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ygl237c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ygl237c, or a functional equivalent or a homolog thereof as depicted in column 8 of Table II, application no. 3, preferably a homolog or functional equivalent as depicted in column 8 of Table II B, application no. 3, and being depicted in the same respective line as said Ygl237c, and preferably the activity is increased non-targeted.

Accordingly, in one embodiment, the molecule which activity is to be increased in the process of the invention is the gene product with an activity as a “ygl237c-protein”, preferably it is the molecule of section (a) or (b) of this paragraph.

In particular, it was observed that in plants, especially in Arabidopsis thaliana, increasing or generating the activity of a gene product non-targeted with the activity of a “ygl237c-protein”, preferably being encoded by a gene comprising the nucleic acid sequence SEQ ID NO. 64177, preferably the coding region thereof, conferred the production of or the increase in tyrosine compared with the wild type control.

The nucleic acid sequence of Ygr221c from Saccharomyces cerevisiae, e.g. as shown in the respective line in column 5 of Table I, application no. 3, has been published in A. And the activity of the gene product thereof is the activity of ygr221 c-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “ygr221 c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ygr221c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ygr221 c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ygr221c, or a functional equivalent or a homolog thereof as depicted in column 8 of Table II, application no. 3, preferably a homolog or functional equivalent as depicted in column 8 of Table II B, application no. 3, and being depicted in the same respective line as said Ygr221 c, and preferably the activity is increased plastidic.

Accordingly, in one embodiment, the molecule which activity is to be increased in the process of the invention is the gene product with an activity as a “ygr221c-protein”, preferably it is the molecule of section (a) or (b) of this paragraph.

In particular, it was observed that in plants, especially in Arabidopsis thaliana, increasing or generating the activity of a gene product plastidic with the activity of a “ygr221 c-protein”, preferably being encoded by a gene comprising the nucleic acid sequence SEQ ID NO. 64315, preferably the coding region thereof, conferred the production of or the increase in aspartate compared with the wild type control.

The nucleic acid sequence of Ygr221c from Saccharomyces cerevisiae, e.g. as shown in the respective line in column 5 of Table I, application no. 3, has been published in A. And the activity of the gene product thereof is the activity of ygr221 c-protein.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “ygr221 c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ygr221c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ygr221 c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ygr221c, or a functional equivalent or a homolog thereof as depicted in column 8 of Table II, application no. 3, preferably a homolog or functional equivalent as depicted in column 8 of Table II B, application no. 3, and being depicted in the same respective line as said Ygr221 c, and preferably the activity is increased plastidic.

Accordingly, in one embodiment, the molecule which activity is to be increased in the process of the invention is the gene product with an activity as a “ygr221c-protein”, preferably it is the molecule of section (a) or (b) of this paragraph.

In particular, it was observed that in plants, especially in Arabidopsis thaliana, increasing or generating the activity of a gene product plastidic with the activity of a “ygr221 c-protein”, preferably being encoded by a gene comprising the nucleic acid sequence SEQ ID NO. 64315, preferably the coding region thereof, conferred the production of or the increase in 5-oxoproline compared with the wild type control.

The nucleic acid sequence of Yhl013c from Saccharomyces cerevisiae, e.g. as shown in the respective line in column 5 of Table I, application no. 3, has been published in A. And the activity of the gene product thereof is the activity of yhl013c-protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “yhl013c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yhl013c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yhl013c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yhl013c, or a functional equivalent or a homolog thereof as depicted in column 8 of Table II, application no. 3, preferably a homolog or functional equivalent as depicted in column 8 of Table II B, application no. 3, and being depicted in the same respective line as said Yhl013c, and preferably the activity is increased plastidic.

Accordingly, in one embodiment, the molecule which activity is to be increased in the process of the invention is the gene product with an activity as a “yhl013c-protein”, preferably it is the molecule of section (a) or (b) of this paragraph.

In particular, it was observed that in plants, especially in Arabidopsis thaliana, increasing or generating the activity of a gene product plastidic with the activity of a “yhl013c-protein”, preferably being encoded by a gene comprising the nucleic acid sequence SEQ ID NO. 14715, preferably the coding region thereof, conferred the production of or the increase in phenylalanine compared with the wild type control.

The nucleic acid sequence of Yhr006w from Saccharomyces cerevisiae, e.g. as shown in the respective line in column 5 of Table I, application no. 3, has been published in A. And the activity of the gene product thereof is the activity of yhr006w-protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “yhr006w-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yhr006w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yhr006w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yhr006w, or a functional equivalent or a homolog thereof as depicted in column 8 of Table II, application no. 3, preferably a homolog or functional equivalent as depicted in column 8 of Table II B, application no. 3, and being depicted in the same respective line as said Yhr006w, and preferably the activity is increased non-targeted.

Accordingly, in one embodiment, the molecule which activity is to be increased in the process of the invention is the gene product with an activity as a “yhr006w-protein”, preferably it is the molecule of section (a) or (b) of this paragraph.

In particular, it was observed that in plants, especially in Arabidopsis thaliana, increasing or generating the activity of a gene product non-targeted with the activity of a “yhr006w-protein”, preferably being encoded by a gene comprising the nucleic acid sequence SEQ ID NO. 90284, preferably the coding region thereof, conferred the production of or the increase in phenylalanine compared with the wild type control.

The nucleic acid sequence of Yhr006w from Saccharomyces cerevisiae, e.g. as shown in the respective line in column 5 of Table I, application no. 3, has been published in A. And the activity of the gene product thereof is the activity of yhr006w-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “yhr006w-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yhr006w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yhr006w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yhr006w, or a functional equivalent or a homolog thereof as depicted in column 8 of Table II, application no. 3, preferably a homolog or functional equivalent as depicted in column 8 of Table II B, application no. 3, and being depicted in the same respective line as said Yhr006w, and preferably the activity is increased non-targeted.

Accordingly, in one embodiment, the molecule which activity is to be increased in the process of the invention is the gene product with an activity as a “yhr006w-protein”, preferably it is the molecule of section (a) or (b) of this paragraph.

In particular, it was observed that in plants, especially in Arabidopsis thaliana, increasing or generating the activity of a gene product non-targeted with the activity of a “yhr006w-protein”, preferably being encoded by a gene comprising the nucleic acid sequence SEQ ID NO. 90284, preferably the coding region thereof, conferred the production of or the increase in tyrosine compared with the wild type control.

The nucleic acid sequence of Yhr207c from Saccharomyces cerevisiae, e.g. as shown in the respective line in column 5 of Table I, application no. 3, has been published in A. And the activity of the gene product thereof is the activity of yhr207c-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “yhr207c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yhr207c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yhr207c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yhr207c, or a functional equivalent or a homolog thereof as depicted in column 8 of Table II, application no. 3, preferably a homolog or functional equivalent as depicted in column 8 of Table II B, application no. 3, and being depicted in the same respective line as said Yhr207c, and preferably the activity is increased plastidic.

Accordingly, in one embodiment, the molecule which activity is to be increased in the process of the invention is the gene product with an activity as a “yhr207c-protein”, preferably it is the molecule of section (a) or (b) of this paragraph.

In particular, it was observed that in plants, especially in Arabidopsis thaliana, increasing or generating the activity of a gene product plastidic with the activity of a “yhr207c-protein”, preferably being encoded by a gene comprising the nucleic acid sequence SEQ ID NO. 64546, preferably the coding region thereof, conferred the production of or the increase in aspartate compared with the wild type control.

The nucleic acid sequence of Yil074c from Saccharomyces cerevisiae, e.g. as shown in the respective line in column 5 of Table I, application no. 3, has been published in A. And the activity of the gene product thereof is the activity of 3-phosphoglycerate dehydrogenase.

Accordingly, in one embodiment, the process of the present invention for producing citrulline in a non-human organism, like a microorganism or a plant or a part thereof, comprises increasing or generating the activity of a gene product with the activity of a gene product conferring the activity of “3-phosphoglycerate dehydrogenase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yil074c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yil074c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yil074c, or a functional equivalent or a homolog thereof as depicted in column 8 of Table II, application no. 3, preferably a homolog or functional equivalent as depicted in column 8 of Table II B, application no. 3, and being depicted in the same respective line as said Yil074c, and preferably the activity is increased plastidic.

Accordingly, in one embodiment, the molecule which activity is to be increased in the process of the invention is the gene product with an activity as a “3-phosphoglycerate dehydrogenase”, preferably it is the molecule of section (a) or (b) of this paragraph.

In particular, it was observed that in plants, especially in Arabidopsis thaliana, increasing or generating the activity of a gene product plastidic with the activity of a “3-phosphoglycerate dehydrogenase”, preferably being encoded by a gene comprising the nucleic acid sequence SEQ ID NO. 64563, preferably the coding region thereof, conferred the production of or the increase in citrulline compared with the wild type control.

The nucleic acid sequence of Yil074c from Saccharomyces cerevisiae, e.g. as shown in the respective line in column 5 of Table I, application no. 3, has been published in A. And the activity of the gene product thereof is the activity of 3-phosphoglycerate dehydrogenase.

Accordingly, in one embodiment, the process of the present invention for producing glycine in a non-human organism, like a microorganism or a plant or a part thereof, comprises increasing or generating the activity of a gene product with the activity of a gene product conferring the activity of “3-phosphoglycerate dehydrogenase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yil074c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yil074c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yil074c, or a functional equivalent or a homolog thereof as depicted in column 8 of Table II, application no. 3, preferably a homolog or functional equivalent as depicted in column 8 of Table II B, application no. 3, and being depicted in the same respective line as said Yil074c, and preferably the activity is increased plastidic.

Accordingly, in one embodiment, the molecule which activity is to be increased in the process of the invention is the gene product with an activity as a “3-phosphoglycerate dehydrogenase”, preferably it is the molecule of section (a) or (b) of this paragraph.

In particular, it was observed that in plants, especially in Arabidopsis thaliana, increasing or generating the activity of a gene product plastidic with the activity of a “3-phosphoglycerate dehydrogenase”, preferably being encoded by a gene comprising the nucleic acid sequence SEQ ID NO. 64563, preferably the coding region thereof, conferred the production of or the increase in glycine compared with the wild type control.

The nucleic acid sequence of Yir038c from Saccharomyces cerevisiae, e.g. as shown in the respective line in column 5 of Table I, application no. 3, has been published in A. And the activity of the gene product thereof is the activity of glutathione S-transferase.

Accordingly, in one embodiment, the process of the present invention for producing aspartate in a non-human organism, like a microorganism or a plant or a part thereof, comprises increasing or generating the activity of a gene product with the activity of a gene product conferring the activity of “glutathione S-transferase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yir038c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yir038c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yir038c, or a functional equivalent or a homolog thereof as depicted in column 8 of Table II, application no. 3, preferably a homolog or functional equivalent as depicted in column 8 of Table II B, application no. 3, and being depicted in the same respective line as said Yir038c, and preferably the activity is increased plastidic.

Accordingly, in one embodiment, the molecule which activity is to be increased in the process of the invention is the gene product with an activity as a “glutathione S-transferase”, preferably it is the molecule of section (a) or (b) of this paragraph.

In particular, it was observed that in plants, especially in Arabidopsis thaliana, increasing or generating the activity of a gene product plastidic with the activity of a “glutathione S-transferase”, preferably being encoded by a gene comprising the nucleic acid sequence SEQ ID NO. 90306, preferably the coding region thereof, conferred the production of or the increase in aspartate compared with the wild type control.

The nucleic acid sequence of Yir038c from Saccharomyces cerevisiae, e.g. as shown in the respective line in column 5 of Table I, application no. 3, has been published in A. And the activity of the gene product thereof is the activity of glutathione S-transferase.

Accordingly, in one embodiment, the process of the present invention for producing citrulline in a non-human organism, like a microorganism or a plant or a part thereof, comprises increasing or generating the activity of a gene product with the activity of a gene product conferring the activity of “glutathione S-transferase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yir038c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yir038c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yir038c, or a functional equivalent or a homolog thereof as depicted in column 8 of Table II, application no. 3, preferably a homolog or functional equivalent as depicted in column 8 of Table II B, application no. 3, and being depicted in the same respective line as said Yir038c, and preferably the activity is increased plastidic.

Accordingly, in one embodiment, the molecule which activity is to be increased in the process of the invention is the gene product with an activity as a “glutathione S-transferase”, preferably it is the molecule of section (a) or (b) of this paragraph.

In particular, it was observed that in plants, especially in Arabidopsis thaliana, increasing or generating the activity of a gene product plastidic with the activity of a “glutathione S-transferase”, preferably being encoded by a gene comprising the nucleic acid sequence SEQ ID NO. 90306, preferably the coding region thereof, conferred the production of or the increase in citrulline compared with the wild type control.

The nucleic acid sequence of Yjl073w from Saccharomyces cerevisiae, e.g. as shown in the respective line in column 5 of Table I, application no. 3, has been published in A. And the activity of the gene product thereof is the activity of DnaJ-like chaperone.

Accordingly, in one embodiment, the process of the present invention for producing aspartate in a non-human organism, like a microorganism or a plant or a part thereof, comprises increasing or generating the activity of a gene product with the activity of a gene product conferring the activity of “DnaJ-like chaperone”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yjl073w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yjl073w, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yj1073w, or a functional equivalent or a homolog thereof as depicted in column 8 of Table II, application no. 3, preferably a homolog or functional equivalent as depicted in column 8 of Table II B, application no. 3, and being depicted in the same respective line as said Yj1073w, and preferably the activity is increased plastidic.

Accordingly, in one embodiment, the molecule which activity is to be increased in the process of the invention is the gene product with an activity as a “DnaJ-like chaperone”, preferably it is the molecule of section (a) or (b) of this paragraph.

In particular, it was observed that in plants, especially in Arabidopsis thaliana, increasing or generating the activity of a gene product plastidic with the activity of a “DnaJ-like chaperone”, preferably being encoded by a gene comprising the nucleic acid sequence SEQ ID NO. 64964, preferably the coding region thereof, conferred the production of or the increase in aspartate compared with the wild type control.

The nucleic acid sequence of Yjr130c from Saccharomyces cerevisiae, e.g. as shown in the respective line in column 5 of Table I, application no. 3, has been published in A. And the activity of the gene product thereof is the activity of cystathionine gamma-synthase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “cystathionine gamma-synthase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yjr130c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yjr130c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yjr130c, or a functional equivalent or a homolog thereof as depicted in column 8 of Table II, application no. 3, preferably a homolog or functional equivalent as depicted in column 8 of Table II B, application no. 3, and being depicted in the same respective line as said Yjr130c, and preferably the activity is increased non-targeted.

Accordingly, in one embodiment, the molecule which activity is to be increased in the process of the invention is the gene product with an activity as a “cystathionine gamma-synthase”, preferably it is the molecule of section (a) or (b) of this paragraph.

In particular, it was observed that in plants, especially in Arabidopsis thaliana, increasing or generating the activity of a gene product non-targeted with the activity of a “cystathionine gamma-synthase”, preferably being encoded by a gene comprising the nucleic acid sequence SEQ ID NO. 14843, preferably the coding region thereof, conferred the production of or the increase in phenylalanine compared with the wild type control.

The nucleic acid sequence of Yjr148w from Saccharomyces cerevisiae, e.g. as shown in the respective line in column 5 of Table I, application no. 3, has been published in A. And the activity of the gene product thereof is the activity of branched chain aminotransferase.

Accordingly, in one embodiment, the process of the present invention for producing citrulline in a non-human organism, like a microorganism or a plant or a part thereof, comprises increasing or generating the activity of a gene product with the activity of a gene product conferring the activity of “branched chain aminotransferase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yjr148w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yjr148w, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yjr148w, or a functional equivalent or a homolog thereof as depicted in column 8 of Table II, application no. 3, preferably a homolog or functional equivalent as depicted in column 8 of Table II B, application no. 3, and being depicted in the same respective line as said Yjr148w, and preferably the activity is increased plastidic.

Accordingly, in one embodiment, the molecule which activity is to be increased in the process of the invention is the gene product with an activity as a “branched chain aminotransferase”, preferably it is the molecule of section (a) or (b) of this paragraph.

In particular, it was observed that in plants, especially in Arabidopsis thaliana, increasing or generating the activity of a gene product plastidic with the activity of a “branched chain aminotransferase”, preferably being encoded by a gene comprising the nucleic acid sequence SEQ ID NO. 90409, preferably the coding region thereof, conferred the production of or the increase in citrulline compared with the wild type control.

The nucleic acid sequence of Yjr153w from Saccharomyces cerevisiae, e.g. as shown in the respective line in column 5 of Table I, application no. 3, has been published in A. And the activity of the gene product thereof is the activity of polygalacturonase.

Accordingly, in one embodiment, the process of the present invention for producing aspartate in a non-human organism, like a microorganism or a plant or a part thereof, comprises increasing or generating the activity of a gene product with the activity of a gene product conferring the activity of “polygalacturonase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yjr153w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yjr153w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yjr153w, or a functional equivalent or a homolog thereof as depicted in column 8 of Table II, application no. 3, preferably a homolog or functional equivalent as depicted in column 8 of Table II B, application no. 3, and being depicted in the same respective line as said Yjr153w, and preferably the activity is increased non-targeted.

Accordingly, in one embodiment, the molecule which activity is to be increased in the process of the invention is the gene product with an activity as a “polygalacturonase”, preferably it is the molecule of section (a) or (b) of this paragraph.

In particular, it was observed that in plants, especially in Arabidopsis thaliana, increasing or generating the activity of a gene product non-targeted with the activity of a “polygalacturonase”, preferably being encoded by a gene comprising the nucleic acid sequence SEQ ID NO. 66274, preferably the coding region thereof, conferred the production of or the increase in aspartate compared with the wild type control.

The nucleic acid sequence of Yjr153w from Saccharomyces cerevisiae, e.g. as shown in the respective line in column 5 of Table I, application no. 3, has been published in A. And the activity of the gene product thereof is the activity of polygalacturonase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “polygalacturonase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yjr153w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yjr153w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yjr153w, or a functional equivalent or a homolog thereof as depicted in column 8 of Table II, application no. 3, preferably a homolog or functional equivalent as depicted in column 8 of Table II B, application no. 3, and being depicted in the same respective line as said Yjr153w, and preferably the activity is increased non-targeted.

Accordingly, in one embodiment, the molecule which activity is to be increased in the process of the invention is the gene product with an activity as a “polygalacturonase”, preferably it is the molecule of section (a) or (b) of this paragraph.

In particular, it was observed that in plants, especially in Arabidopsis thaliana, increasing or generating the activity of a gene product non-targeted with the activity of a “polygalacturonase”, preferably being encoded by a gene comprising the nucleic acid sequence SEQ ID NO. 66274, preferably the coding region thereof, conferred the production of or the increase in phenylalanine compared with the wild type control.

The nucleic acid sequence of YKL038W from SACCHAROMYCES CEREVISIAE, e.g. as shown in the respective line in column 5 of Table I, application no. 3, has been published in A. And the activity of the gene product thereof is the activity of YKL038W-protein.

Accordingly, in one embodiment, the process of the present invention for producing alanine in a non-human organism, like a microorganism or a plant or a part thereof, comprises increasing or generating the activity of a gene product with the activity of a gene product conferring the activity of “YKL038W-protein”, especially from SACCHAROMYCES CEREVISIAE or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said YKL038W, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said YKL038W, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said YKL038W, or a functional equivalent or a homolog thereof as depicted in column 8 of Table II, application no. 3, preferably a homolog or functional equivalent as depicted in column 8 of Table II B, application no. 3, and being depicted in the same respective line as said YKL038W, and preferably the activity is increased plastidic.

Accordingly, in one embodiment, the molecule which activity is to be increased in the process of the invention is the gene product with an activity as a “YKL038W-protein”, preferably it is the molecule of section (a) or (b) of this paragraph.

In particular, it was observed that in plants, especially in Arabidopsis thaliana, increasing or generating the activity of a gene product plastidic with the activity of a “YKL038W-protein”, preferably being encoded by a gene comprising the nucleic acid sequence SEQ ID NO. 90602, preferably the coding region thereof, conferred the production of or the increase in alanine compared with the wild type control.

The nucleic acid sequence of YKL038W from SACCHAROMYCES CEREVISIAE, e.g. as shown in the respective line in column 5 of Table I, application no. 3, has been published in A. And the activity of the gene product thereof is the activity of YKL038W-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “YKL038W-protein”, especially from SACCHAROMYCES CEREVISIAE or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said YKL038W, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said YKL038W, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said YKL038W, or a functional equivalent or a homolog thereof as depicted in column 8 of Table II, application no. 3, preferably a homolog or functional equivalent as depicted in column 8 of Table II B, application no. 3, and being depicted in the same respective line as said YKL038W, and preferably the activity is increased plastidic.

Accordingly, in one embodiment, the molecule which activity is to be increased in the process of the invention is the gene product with an activity as a “YKL038W-protein”, preferably it is the molecule of section (a) or (b) of this paragraph.

In particular, it was observed that in plants, especially in Arabidopsis thaliana, increasing or generating the activity of a gene product plastidic with the activity of a “YKL038W-protein”, preferably being encoded by a gene comprising the nucleic acid sequence SEQ ID NO. 90602, preferably the coding region thereof, conferred the production of or the increase in aspartate compared with the wild type control.

The nucleic acid sequence of YKL038W from SACCHAROMYCES CEREVISIAE, e.g. as shown in the respective line in column 5 of Table I, application no. 3, has been published in A. And the activity of the gene product thereof is the activity of YKL038W-protein.

Accordingly, in one embodiment, the process of the present invention for producing serine in a non-human organism, like a microorganism or a plant or a part thereof, comprises increasing or generating the activity of a gene product with the activity of a gene product conferring the activity of “YKL038W-protein”, especially from SACCHAROMYCES CEREVISIAE or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said YKL038W, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said YKL038W, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said YKL038W, or a functional equivalent or a homolog thereof as depicted in column 8 of Table II, application no. 3, preferably a homolog or functional equivalent as depicted in column 8 of Table II B, application no. 3, and being depicted in the same respective line as said YKL038W, and preferably the activity is increased plastidic.

Accordingly, in one embodiment, the molecule which activity is to be increased in the process of the invention is the gene product with an activity as a “YKL038W-protein”, preferably it is the molecule of section (a) or (b) of this paragraph.

In particular, it was observed that in plants, especially in Arabidopsis thaliana, increasing or generating the activity of a gene product plastidic with the activity of a “YKL038W-protein”, preferably being encoded by a gene comprising the nucleic acid sequence SEQ ID NO. 90602, preferably the coding region thereof, conferred the production of or the increase in serine compared with the wild type control.

The nucleic acid sequence of Ylr027c from Saccharomyces cerevisiae, e.g. as shown in the respective line in column 5 of Table I, application no. 3, has been published in A. And the activity of the gene product thereof is the activity of aspartate aminotransferase.

Accordingly, in one embodiment, the process of the present invention for producing glycine in a non-human organism, like a microorganism or a plant or a part thereof, comprises increasing or generating the activity of a gene product with the activity of a gene product conferring the activity of “aspartate aminotransferase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ylr027c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ylr027c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ylr027c, or a functional equivalent or a homolog thereof as depicted in column 8 of Table II, application no. 3, preferably a homolog or functional equivalent as depicted in column 8 of Table II B, application no. 3, and being depicted in the same respective line as said Ylr027c, and preferably the activity is increased non-targeted.

Accordingly, in one embodiment, the molecule which activity is to be increased in the process of the invention is the gene product with an activity as a “aspartate aminotransferase”, preferably it is the molecule of section (a) or (b) of this paragraph.

In particular, it was observed that in plants, especially in Arabidopsis thaliana, increasing or generating the activity of a gene product non-targeted with the activity of a “aspartate aminotransferase”, preferably being encoded by a gene comprising the nucleic acid sequence SEQ ID NO. 90615, preferably the coding region thereof, conferred the production of or the increase in glycine compared with the wild type control.

The nucleic acid sequence of Ylr043c from Saccharomyces cerevisiae, e.g. as shown in the respective line in column 5 of Table I, application no. 3, has been published in A. And the activity of the gene product thereof is the activity of thioredoxin.

Accordingly, in one embodiment, the process of the present invention for producing serine in a non-human organism, like a microorganism or a plant or a part thereof, comprises increasing or generating the activity of a gene product with the activity of a gene product conferring the activity of “thioredoxin”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ylr043c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ylr043c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ylr043c, or a functional equivalent or a homolog thereof as depicted in column 8 of Table II, application no. 3, preferably a homolog or functional equivalent as depicted in column 8 of Table II B, application no. 3, and being depicted in the same respective line as said Ylr043c, and preferably the activity is increased plastidic.

Accordingly, in one embodiment, the molecule which activity is to be increased in the process of the invention is the gene product with an activity as a “thioredoxin”, preferably it is the molecule of section (a) or (b) of this paragraph.

In particular, it was observed that in plants, especially in Arabidopsis thaliana, increasing or generating the activity of a gene product plastidic with the activity of a “thioredoxin”, preferably being encoded by a gene comprising the nucleic acid sequence SEQ ID NO. 91045, preferably the coding region thereof, conferred the production of or the increase in serine compared with the wild type control.

The nucleic acid sequence of Ylr043c from Saccharomyces cerevisiae, e.g. as shown in the respective line in column 5 of Table I, application no. 3, has been published in A. And the activity of the gene product thereof is the activity of thioredoxin.

Accordingly, in one embodiment, the process of the present invention for producing tyrosine in a non-human organism, like a microorganism or a plant or a part thereof, comprises increasing or generating the activity of a gene product with the activity of a gene product conferring the activity of “thioredoxin”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ylr043c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ylr043c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ylr043c, or a functional equivalent or a homolog thereof as depicted in column 8 of Table II, application no. 3, preferably a homolog or functional equivalent as depicted in column 8 of Table II B, application no. 3, and being depicted in the same respective line as said Ylr043c, and preferably the activity is increased plastidic.

Accordingly, in one embodiment, the molecule which activity is to be increased in the process of the invention is the gene product with an activity as a “thioredoxin”, preferably it is the molecule of section (a) or (b) of this paragraph.

In particular, it was observed that in plants, especially in Arabidopsis thaliana, increasing or generating the activity of a gene product plastidic with the activity of a “thioredoxin”, preferably being encoded by a gene comprising the nucleic acid sequence SEQ ID NO. 91045, preferably the coding region thereof, conferred the production of or the increase in tyrosine compared with the wild type control.

The nucleic acid sequence of Yml008c from Saccharomyces cerevisiae, e.g. as shown in the respective line in column 5 of Table I, application no. 3, has been published in A. And the activity of the gene product thereof is the activity of sterol 24-C-methyltransferase.

Accordingly, in one embodiment, the process of the present invention for producing alanine in a non-human organism, like a microorganism or a plant or a part thereof, comprises increasing or generating the activity of a gene product with the activity of a gene product conferring the activity of “sterol 24-C-methyltransferase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yml008c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yml008c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yml008c, or a functional equivalent or a homolog thereof as depicted in column 8 of Table II, application no. 3, preferably a homolog or functional equivalent as depicted in column 8 of Table II B, application no. 3, and being depicted in the same respective line as said Yml008c, and preferably the activity is increased plastidic.

Accordingly, in one embodiment, the molecule which activity is to be increased in the process of the invention is the gene product with an activity as a “sterol 24-C-methyltransferase”, preferably it is the molecule of section (a) or (b) of this paragraph.

In particular, it was observed that in plants, especially in Arabidopsis thaliana, increasing or generating the activity of a gene product plastidic with the activity of a “sterol 24-C-methyltransferase”, preferably being encoded by a gene comprising the nucleic acid sequence SEQ ID NO. 91659, preferably the coding region thereof, conferred the production of or the increase in alanine compared with the wild type control.

The nucleic acid sequence of Yml008c from Saccharomyces cerevisiae, e.g. as shown in the respective line in column 5 of Table I, application no. 3, has been published in A. And the activity of the gene product thereof is the activity of sterol 24-C-methyltransferase.

Accordingly, in one embodiment, the process of the present invention for producing glycine in a non-human organism, like a microorganism or a plant or a part thereof, comprises increasing or generating the activity of a gene product with the activity of a gene product conferring the activity of “sterol 24-C-methyltransferase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yml008c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yml008c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yml008c, or a functional equivalent or a homolog thereof as depicted in column 8 of Table II, application no. 3, preferably a homolog or functional equivalent as depicted in column 8 of Table II B, application no. 3, and being depicted in the same respective line as said Yml008c, and preferably the activity is increased plastidic.

Accordingly, in one embodiment, the molecule which activity is to be increased in the process of the invention is the gene product with an activity as a “sterol 24-C-methyltransferase”, preferably it is the molecule of section (a) or (b) of this paragraph.

In particular, it was observed that in plants, especially in Arabidopsis thaliana, increasing or generating the activity of a gene product plastidic with the activity of a “sterol 24-C-methyltransferase”, preferably being encoded by a gene comprising the nucleic acid sequence SEQ ID NO. 91659, preferably the coding region thereof, conferred the production of or the increase in glycine compared with the wild type control.

The nucleic acid sequence of Yml008c from Saccharomyces cerevisiae, e.g. as shown in the respective line in column 5 of Table I, application no. 3, has been published in A. And the activity of the gene product thereof is the activity of sterol 24-C-methyltransferase.

Accordingly, in one embodiment, the process of the present invention for producing homoserine in a non-human organism, like a microorganism or a plant or a part thereof, comprises increasing or generating the activity of a gene product with the activity of a gene product conferring the activity of “sterol 24-C-methyltransferase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yml008c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yml008c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yml008c, or a functional equivalent or a homolog thereof as depicted in column 8 of Table II, application no. 3, preferably a homolog or functional equivalent as depicted in column 8 of Table II B, application no. 3, and being depicted in the same respective line as said Yml008c, and preferably the activity is increased plastidic.

Accordingly, in one embodiment, the molecule which activity is to be increased in the process of the invention is the gene product with an activity as a “sterol 24-C-methyltransferase”, preferably it is the molecule of section (a) or (b) of this paragraph.

In particular, it was observed that in plants, especially in Arabidopsis thaliana, increasing or generating the activity of a gene product plastidic with the activity of a “sterol 24-C-methyltransferase”, preferably being encoded by a gene comprising the nucleic acid sequence SEQ ID NO. 91659, preferably the coding region thereof, conferred the production of or the increase in homoserine compared with the wild type control.

The nucleic acid sequence of Yml008c from Saccharomyces cerevisiae, e.g. as shown in the respective line in column 5 of Table I, application no. 3, has been published in A. And the activity of the gene product thereof is the activity of sterol 24-C-methyltransferase.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “sterol 24-C-methyltransferase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yml008c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yml008c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yml008c, or a functional equivalent or a homolog thereof as depicted in column 8 of Table II, application no. 3, preferably a homolog or functional equivalent as depicted in column 8 of Table II B, application no. 3, and being depicted in the same respective line as said Yml008c, and preferably the activity is increased plastidic.

Accordingly, in one embodiment, the molecule which activity is to be increased in the process of the invention is the gene product with an activity as a “sterol 24-C-methyltransferase”, preferably it is the molecule of section (a) or (b) of this paragraph.

In particular, it was observed that in plants, especially in Arabidopsis thaliana, increasing or generating the activity of a gene product plastidic with the activity of a “sterol 24-C-methyltransferase”, preferably being encoded by a gene comprising the nucleic acid sequence SEQ ID NO. 91659, preferably the coding region thereof, conferred the production of or the increase in 5-oxoproline compared with the wild type control.

The nucleic acid sequence of Yml083c from Saccharomyces cerevisiae, e.g. as shown in the respective line in column 5 of Table I, application no. 3, has been published in A. And the activity of the gene product thereof is the activity of yml083c-protein.

Accordingly, in one embodiment, the process of the present invention for producing alanine in a non-human organism, like a microorganism or a plant or a part thereof, comprises increasing or generating the activity of a gene product with the activity of a gene product conferring the activity of “yml083c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yml083c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yml083c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yml083c, or a functional equivalent or a homolog thereof as depicted in column 8 of Table II, application no. 3, preferably a homolog or functional equivalent as depicted in column 8 of Table II B, application no. 3, and being depicted in the same respective line as said Yml083c, and preferably the activity is increased plastidic.

Accordingly, in one embodiment, the molecule which activity is to be increased in the process of the invention is the gene product with an activity as a “yml083c-protein”, preferably it is the molecule of section (a) or (b) of this paragraph.

In particular, it was observed that in plants, especially in Arabidopsis thaliana, increasing or generating the activity of a gene product plastidic with the activity of a “yml083c-protein”, preferably being encoded by a gene comprising the nucleic acid sequence SEQ ID NO. 91781, preferably the coding region thereof, conferred the production of or the increase in alanine compared with the wild type control.

The nucleic acid sequence of Yml123c from Saccharomyces cerevisiae, e.g. as shown in the respective line in column 5 of Table I, application no. 3, has been published in A. And the activity of the gene product thereof is the activity of phosphate transporter.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “phosphate transporter”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yml123c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yml123c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yml123c, or a functional equivalent or a homolog thereof as depicted in column 8 of Table II, application no. 3, preferably a homolog or functional equivalent as depicted in column 8 of Table II B, application no. 3, and being depicted in the same respective line as said Yml123c, and preferably the activity is increased plastidic.

Accordingly, in one embodiment, the molecule which activity is to be increased in the process of the invention is the gene product with an activity as a “phosphate transporter”, preferably it is the molecule of section (a) or (b) of this paragraph.

In particular, it was observed that in plants, especially in Arabidopsis thaliana, increasing or generating the activity of a gene product plastidic with the activity of a “phosphate transporter”, preferably being encoded by a gene comprising the nucleic acid sequence SEQ ID NO. 91785, preferably the coding region thereof, conferred the production of or the increase in phenylalanine compared with the wild type control.

The nucleic acid sequence of Yml123c from Saccharomyces cerevisiae, e.g. as shown in the respective line in column 5 of Table I, application no. 3, has been published in A. And the activity of the gene product thereof is the activity of phosphate transporter.

Accordingly, in one embodiment, the process of the present invention for producing tyrosine in a non-human organism, like a microorganism or a plant or a part thereof, comprises increasing or generating the activity of a gene product with the activity of a gene product conferring the activity of “phosphate transporter”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yml123c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yml123c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yml123c, or a functional equivalent or a homolog thereof as depicted in column 8 of Table II, application no. 3, preferably a homolog or functional equivalent as depicted in column 8 of Table II B, application no. 3, and being depicted in the same respective line as said Yml123c, and preferably the activity is increased plastidic.

Accordingly, in one embodiment, the molecule which activity is to be increased in the process of the invention is the gene product with an activity as a “phosphate transporter”, preferably it is the molecule of section (a) or (b) of this paragraph.

In particular, it was observed that in plants, especially in Arabidopsis thaliana, increasing or generating the activity of a gene product plastidic with the activity of a “phosphate transporter”, preferably being encoded by a gene comprising the nucleic acid sequence SEQ ID NO. 91785, preferably the coding region thereof, conferred the production of or the increase in tyrosine compared with the wild type control.

The nucleic acid sequence of Ymr013c from Saccharomyces cerevisiae, e.g. as shown in the respective line in column 5 of Table I, application no. 3, has been published in A. And the activity of the gene product thereof is the activity of ymr013c-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “ymr013c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ymr013c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ymr013c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ymr013c, or a functional equivalent or a homolog thereof as depicted in column 8 of Table II, application no. 3, preferably a homolog or functional equivalent as depicted in column 8 of Table II B, application no. 3, and being depicted in the same respective line as said Ymr013c, and preferably the activity is increased plastidic.

Accordingly, in one embodiment, the molecule which activity is to be increased in the process of the invention is the gene product with an activity as a “ymr013c-protein”, preferably it is the molecule of section (a) or (b) of this paragraph.

In particular, it was observed that in plants, especially in Arabidopsis thaliana, increasing or generating the activity of a gene product plastidic with the activity of a “ymr013c-protein”, preferably being encoded by a gene comprising the nucleic acid sequence SEQ ID NO. 91958, preferably the coding region thereof, conferred the production of or the increase in tyrosine compared with the wild type control.

The nucleic acid sequence of Ymr120c from Saccharomyces cerevisiae, e.g. as shown in the respective line in column 5 of Table I, application no. 3, has been published in A. And the activity of the gene product thereof is the activity of purine biosynthesis protein.

Accordingly, in one embodiment, the process of the present invention for producing glycine in a non-human organism, like a microorganism or a plant or a part thereof, comprises increasing or generating the activity of a gene product with the activity of a gene product conferring the activity of “purine biosynthesis protein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ymr120c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ymr120c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ymr120c, or a functional equivalent or a homolog thereof as depicted in column 8 of Table II, application no. 3, preferably a homolog or functional equivalent as depicted in column 8 of Table II B, application no. 3, and being depicted in the same respective line as said Ymr120c, and preferably the activity is increased plastidic.

Accordingly, in one embodiment, the molecule which activity is to be increased in the process of the invention is the gene product with an activity as a “purine biosynthesis protein”, preferably it is the molecule of section (a) or (b) of this paragraph.

In particular, it was observed that in plants, especially in Arabidopsis thaliana, increasing or generating the activity of a gene product plastidic with the activity of a “purine biosynthesis protein”, preferably being encoded by a gene comprising the nucleic acid sequence SEQ ID NO. 91973, preferably the coding region thereof, conferred the production of or the increase in glycine compared with the wild type control.

The nucleic acid sequence of Ymr150c from Saccharomyces cerevisiae, e.g. as shown in the respective line in column 5 of Table I, application no. 3, has been published in A. And the activity of the gene product thereof is the activity of mitochondrial inner membrane protease subunit.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “mitochondrial inner membrane protease subunit”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ymr150c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ymr150c, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ymr150c, or a functional equivalent or a homolog thereof as depicted in column 8 of Table II, application no. 3, preferably a homolog or functional equivalent as depicted in column 8 of Table II B, application no. 3, and being depicted in the same respective line as said Ymr150c, and preferably the activity is increased plastidic.

Accordingly, in one embodiment, the molecule which activity is to be increased in the process of the invention is the gene product with an activity as a “mitochondrial inner membrane protease subunit”, preferably it is the molecule of section (a) or (b) of this paragraph.

In particular, it was observed that in plants, especially in Arabidopsis thaliana, increasing or generating the activity of a gene product plastidic with the activity of a “mitochondrial inner membrane protease subunit”, preferably being encoded by a gene comprising the nucleic acid sequence SEQ ID NO. 92274, preferably the coding region thereof, conferred the production of or the increase in phenylalanine compared with the wild type control.

The nucleic acid sequence of Ynl086w from Saccharomyces cerevisiae, e.g. as shown in the respective line in column 5 of Table I, application no. 3, has been published in A. And the activity of the gene product thereof is the activity of YNL086W-protein.

Accordingly, in one embodiment, the process of the present invention for producing homoserine in a non-human organism, like a microorganism or a plant or a part thereof, comprises increasing or generating the activity of a gene product with the activity of a gene product conferring the activity of “YNL086W-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ynl086w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ynl086w, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ynl086w, or a functional equivalent or a homolog thereof as depicted in column 8 of Table II, application no. 3, preferably a homolog or functional equivalent as depicted in column 8 of Table II B, application no. 3, and being depicted in the same respective line as said Ynl086w, and preferably the activity is increased plastidic.

Accordingly, in one embodiment, the molecule which activity is to be increased in the process of the invention is the gene product with an activity as a “YNL086W-protein”, preferably it is the molecule of section (a) or (b) of this paragraph.

In particular, it was observed that in plants, especially in Arabidopsis thaliana, increasing or generating the activity of a gene product plastidic with the activity of a “YNL086W-protein”, preferably being encoded by a gene comprising the nucleic acid sequence SEQ ID NO. 92325, preferably the coding region thereof, conferred the production of or the increase in homoserine compared with the wild type control.

The nucleic acid sequence of Ynl101w from Saccharomyces cerevisiae, e.g. as shown in the respective line in column 5 of Table I, application no. 3, has been published in A. And the activity of the gene product thereof is the activity of amino acid transporter.

Accordingly, in one embodiment, the process of the present invention for producing tyrosine in a non-human organism, like a microorganism or a plant or a part thereof, comprises increasing or generating the activity of a gene product with the activity of a gene product conferring the activity of “amino acid transporter”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ynl101w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ynl101w, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ynl101w, or a functional equivalent or a homolog thereof as depicted in column 8 of Table II, application no. 3, preferably a homolog or functional equivalent as depicted in column 8 of Table II B, application no. 3, and being depicted in the same respective line as said Ynl101w, and preferably the activity is increased plastidic.

Accordingly, in one embodiment, the molecule which activity is to be increased in the process of the invention is the gene product with an activity as a “amino acid transporter”, preferably it is the molecule of section (a) or (b) of this paragraph.

In particular, it was observed that in plants, especially in Arabidopsis thaliana, increasing or generating the activity of a gene product plastidic with the activity of a “amino acid transporter”, preferably being encoded by a gene comprising the nucleic acid sequence SEQ ID NO. 92339, preferably the coding region thereof, conferred the production of or the increase in tyrosine compared with the wild type control.

The nucleic acid sequence of Ynl277w from Saccharomyces cerevisiae, e.g. as shown in the respective line in column 5 of Table I, application no. 3, has been published in A. And the activity of the gene product thereof is the activity of homoserine O-acetyltransferase.

Accordingly, in one embodiment, the process of the present invention for producing homoserine in a non-human organism, like a microorganism or a plant or a part thereof, comprises increasing or generating the activity of a gene product with the activity of a gene product conferring the activity of “homoserine 0-acetyltransferase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ynl277w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ynl277w, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ynl277w, or a functional equivalent or a homolog thereof as depicted in column 8 of Table II, application no. 3, preferably a homolog or functional equivalent as depicted in column 8 of Table II B, application no. 3, and being depicted in the same respective line as said Ynl277w, and preferably the activity is increased plastidic.

Accordingly, in one embodiment, the molecule which activity is to be increased in the process of the invention is the gene product with an activity as a “homoserine O-acetyltransferase”, preferably it is the molecule of section (a) or (b) of this paragraph.

In particular, it was observed that in plants, especially in Arabidopsis thaliana, increasing or generating the activity of a gene product plastidic with the activity of a “homoserine O-acetyltransferase”, preferably being encoded by a gene comprising the nucleic acid sequence SEQ ID NO. 92391, preferably the coding region thereof, conferred the production of or the increase in homoserine compared with the wild type control.

The nucleic acid sequence of Yol130w from Saccharomyces cerevisiae, e.g. as shown in the respective line in column 5 of Table I, application no. 3, has been published in A. And the activity of the gene product thereof is the activity of metal ion transporter.

Accordingly, in one embodiment, the process of the present invention for producing alanine in a non-human organism, like a microorganism or a plant or a part thereof, comprises increasing or generating the activity of a gene product with the activity of a gene product conferring the activity of “metal ion transporter”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yol130w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yol130w, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yol130w, or a functional equivalent or a homolog thereof as depicted in column 8 of Table II, application no. 3, preferably a homolog or functional equivalent as depicted in column 8 of Table II B, application no. 3, and being depicted in the same respective line as said Yol130w, and preferably the activity is increased plastidic.

Accordingly, in one embodiment, the molecule which activity is to be increased in the process of the invention is the gene product with an activity as a “metal ion transporter”, preferably it is the molecule of section (a) or (b) of this paragraph.

In particular, it was observed that in plants, especially in Arabidopsis thaliana, increasing or generating the activity of a gene product plastidic with the activity of a “metal ion transporter”, preferably being encoded by a gene comprising the nucleic acid sequence SEQ ID NO. 92510, preferably the coding region thereof, conferred the production of or the increase in alanine compared with the wild type control.

The nucleic acid sequence of Yol160w from Saccharomyces cerevisiae, e.g. as shown in the respective line in column 5 of Table I, application no. 3, has been published in A. And the activity of the gene product thereof is the activity of yol160w-protein.

Accordingly, in one embodiment, the process of the present invention for producing aspartate in a non-human organism, like a microorganism or a plant or a part thereof, comprises increasing or generating the activity of a gene product with the activity of a gene product conferring the activity of “yol160w-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yol160w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yol160w, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yol160w, or a functional equivalent or a homolog thereof as depicted in column 8 of Table II, application no. 3, preferably a homolog or functional equivalent as depicted in column 8 of Table II B, application no. 3, and being depicted in the same respective line as said Yol160w, and preferably the activity is increased plastidic.

Accordingly, in one embodiment, the molecule which activity is to be increased in the process of the invention is the gene product with an activity as a “yol160w-protein”, preferably it is the molecule of section (a) or (b) of this paragraph.

In particular, it was observed that in plants, especially in Arabidopsis thaliana, increasing or generating the activity of a gene product plastidic with the activity of a “yol160w-protein”, preferably being encoded by a gene comprising the nucleic acid sequence SEQ ID NO. 15179, preferably the coding region thereof, conferred the production of or the increase in aspartate compared with the wild type control.

The nucleic acid sequence of Yor233w from Saccharomyces cerevisiae, e.g. as shown in the respective line in column 5 of Table I, application no. 3, has been published in A. And the activity of the gene product thereof is the activity of protein kinase.

Accordingly, in one embodiment, the process of the present invention for producing citrulline in a non-human organism, like a microorganism or a plant or a part thereof, comprises increasing or generating the activity of a gene product with the activity of a gene product conferring the activity of “protein kinase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yor233w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yor233w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yor233w, or a functional equivalent or a homolog thereof as depicted in column 8 of Table II, application no. 3, preferably a homolog or functional equivalent as depicted in column 8 of Table II B, application no. 3, and being depicted in the same respective line as said Yor233w, and preferably the activity is increased non-targeted.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 67968, preferably the coding region thereof, conferred the production of or the increase in citrulline compared with the wild type control.

The nucleic acid sequence of Yor244w from Saccharomyces cerevisiae, e.g. as shown in the respective line in column 5 of Table I, application no. 3, has been published in A. And the activity of the gene product thereof is the activity of histone acetyltransferase.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “histone acetyltransferase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yor244w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yor244w, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yor244w, or a functional equivalent or a homolog thereof as depicted in column 8 of Table II, application no. 3, preferably a homolog or functional equivalent as depicted in column 8 of Table II B, application no. 3, and being depicted in the same respective line as said Yor244w, and preferably the activity is increased plastidic.

Accordingly, in one embodiment, the molecule which activity is to be increased in the process of the invention is the gene product with an activity as a “histone acetyltransferase”, preferably it is the molecule of section (a) or (b) of this paragraph.

In particular, it was observed that in plants, especially in Arabidopsis thaliana, increasing or generating the activity of a gene product plastidic with the activity of a “histone acetyltransferase”, preferably being encoded by a gene comprising the nucleic acid sequence SEQ ID NO. 92540, preferably the coding region thereof, conferred the production of or the increase in phenylalanine compared with the wild type control.

The nucleic acid sequence of Yor244w from Saccharomyces cerevisiae, e.g. as shown in the respective line in column 5 of Table I, application no. 3, has been published in A. And the activity of the gene product thereof is the activity of histone acetyltransferase.

Accordingly, in one embodiment, the process of the present invention for producing tyrosine in a non-human organism, like a microorganism or a plant or a part thereof, comprises increasing or generating the activity of a gene product with the activity of a gene product conferring the activity of “histone acetyltransferase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yor244w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yor244w, and preferably the activity is increased plastidic, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yor244w, or a functional equivalent or a homolog thereof as depicted in column 8 of Table II, application no. 3, preferably a homolog or functional equivalent as depicted in column 8 of Table II B, application no. 3, and being depicted in the same respective line as said Yor244w, and preferably the activity is increased plastidic.

Accordingly, in one embodiment, the molecule which activity is to be increased in the process of the invention is the gene product with an activity as a “histone acetyltransferase”, preferably it is the molecule of section (a) or (b) of this paragraph.

In particular, it was observed that in plants, especially in Arabidopsis thaliana, increasing or generating the activity of a gene product plastidic with the activity of a “histone acetyltransferase”, preferably being encoded by a gene comprising the nucleic acid sequence SEQ ID NO. 92540, preferably the coding region thereof, conferred the production of or the increase in tyrosine compared with the wild type control.

The nucleic acid sequence of Yor245c from Saccharomyces cerevisiae, e.g. as shown in the respective line in column 5 of Table I, application no. 3, has been published in A. And the activity of the gene product thereof is the activity of diacylglycerol O-acyltransferase.

Accordingly, in one embodiment, the process of the present invention for producing homoserine in a non-human organism, like a microorganism or a plant or a part thereof, comprises increasing or generating the activity of a gene product with the activity of a gene product conferring the activity of “diacylglycerol O-acyltransferase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yor245c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yor245c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yor245c, or a functional equivalent or a homolog thereof as depicted in column 8 of Table II, application no. 3, preferably a homolog or functional equivalent as depicted in column 8 of Table II B, application no. 3, and being depicted in the same respective line as said Yor245c, and preferably the activity is increased non-targeted.

Accordingly, in one embodiment, the molecule which activity is to be increased in the process of the invention is the gene product with an activity as a “diacylglycerol O-acyltransferase”, preferably it is the molecule of section (a) or (b) of this paragraph.

In particular, it was observed that in plants, especially in Arabidopsis thaliana, increasing or generating the activity of a gene product non-targeted with the activity of a “diacylglycerol O-acyltransferase”, preferably being encoded by a gene comprising the nucleic acid sequence SEQ ID NO. 92616, preferably the coding region thereof, conferred the production of or the increase in homoserine compared with the wild type control.

The nucleic acid sequence of Yor245c from Saccharomyces cerevisiae, e.g. as shown in the respective line in column 5 of Table I, application no. 3, has been published in A. And the activity of the gene product thereof is the activity of diacylglycerol O-acyltransferase.

Accordingly, in one embodiment, the process of the present invention for producing serine in a non-human organism, like a microorganism or a plant or a part thereof, comprises increasing or generating the activity of a gene product with the activity of a gene product conferring the activity of “diacylglycerol O-acyltransferase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yor245c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yor245c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yor245c, or a functional equivalent or a homolog thereof as depicted in column 8 of Table II, application no. 3, preferably a homolog or functional equivalent as depicted in column 8 of Table II B, application no. 3, and being depicted in the same respective line as said Yor245c, and preferably the activity is increased non-targeted.

Accordingly, in one embodiment, the molecule which activity is to be increased in the process of the invention is the gene product with an activity as a “diacylglycerol O-acyltransferase”, preferably it is the molecule of section (a) or (b) of this paragraph.

In particular, it was observed that in plants, especially in Arabidopsis thaliana, increasing or generating the activity of a gene product non-targeted with the activity of a “diacylglycerol O-acyltransferase”, preferably being encoded by a gene comprising the nucleic acid sequence SEQ ID NO. 92616, preferably the coding region thereof, conferred the production of or the increase in serine compared with the wild type control.

The nucleic acid sequence of Yor245c from Saccharomyces cerevisiae, e.g. as shown in the respective line in column 5 of Table I, application no. 3, has been published in A. And the activity of the gene product thereof is the activity of diacylglycerol O-acyltransferase.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “diacylglycerol O-acyltransferase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Yor245c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Yor245c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Yor245c, or a functional equivalent or a homolog thereof as depicted in column 8 of Table II, application no. 3, preferably a homolog or functional equivalent as depicted in column 8 of Table II B, application no. 3, and being depicted in the same respective line as said Yor245c, and preferably the activity is increased non-targeted.

Accordingly, in one embodiment, the molecule which activity is to be increased in the process of the invention is the gene product with an activity as a “diacylglycerol O-acyltransferase”, preferably it is the molecule of section (a) or (b) of this paragraph.

In particular, it was observed that in plants, especially in Arabidopsis thaliana, increasing or generating the activity of a gene product non-targeted with the activity of a “diacylglycerol O-acyltransferase”, preferably being encoded by a gene comprising the nucleic acid sequence SEQ ID NO. 92616, preferably the coding region thereof, conferred the production of or the increase in 5-oxoproline compared with the wild type control.

The nucleic acid sequence of Ypl028w from Saccharomyces cerevisiae, e.g. as shown in the respective line in column 5 of Table I, application no. 3, has been published in A. And the activity of the gene product thereof is the activity of acetyl-CoA acetyltransferase.

Accordingly, in one embodiment, the process of the present invention for producing glycine in a non-human organism, like a microorganism or a plant or a part thereof, comprises increasing or generating the activity of a gene product with the activity of a gene product conferring the activity of “acetyl-CoA acetyltransferase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ypl028w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ypl028w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ypl028w, or a functional equivalent or a homolog thereof as depicted in column 8 of Table II, application no. 3, preferably a homolog or functional equivalent as depicted in column 8 of Table II B, application no. 3, and being depicted in the same respective line as said Ypl028w, and preferably the activity is increased non-targeted.

Accordingly, in one embodiment, the molecule which activity is to be increased in the process of the invention is the gene product with an activity as a “acetyl-CoA acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyl-CoA acetyltransferase”, preferably being encoded by a gene comprising the nucleic acid sequence SEQ ID NO. 92668, preferably the coding region thereof, conferred the production of or the increase in glycine compared with the wild type control.

The nucleic acid sequence of Ypr001w from Saccharomyces cerevisiae, e.g. as shown in the respective line in column 5 of Table I, application no. 3, has been published in A. And the activity of the gene product thereof is the activity of citrate synthase.

Accordingly, in one embodiment, the process of the present invention for producing serine in a non-human organism, like a microorganism or a plant or a part thereof, comprises increasing or generating the activity of a gene product with the activity of a gene product conferring the activity of “citrate synthase”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ypr001w, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ypr001w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ypr001w, or a functional equivalent or a homolog thereof as depicted in column 8 of Table II, application no. 3, preferably a homolog or functional equivalent as depicted in column 8 of Table II B, application no. 3, and being depicted in the same respective line as said Ypr001w, and preferably the activity is increased non-targeted.

Accordingly, in one embodiment, the molecule which activity is to be increased in the process of the invention is the gene product with an activity as a “citrate synthase”, preferably it is the molecule of section (a) or (b) of this paragraph.

In particular, it was observed that in plants, especially in Arabidopsis thaliana, increasing or generating the activity of a gene product non-targeted with the activity of a “citrate synthase”, preferably being encoded by a gene comprising the nucleic acid sequence SEQ ID NO. 93091, preferably the coding region thereof, conferred the production of or the increase in serine compared with the wild type control.

The nucleic acid sequence of Ypr138c from Saccharomyces cerevisiae, e.g. as shown in the respective line in column 5 of Table I, application no. 3, has been published in A. And the activity of the gene product thereof is the activity of ammonium transporter.

Accordingly, in one embodiment, the process of the present invention for producing aspartate in a non-human organism, like a microorganism or a plant or a part thereof, comprises increasing or generating the activity of a gene product with the activity of a gene product conferring the activity of “ammonium transporter”, especially from Saccharomyces cerevisiae or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Ypr138c, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Ypr138c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Ypr138c, or a functional equivalent or a homolog thereof as depicted in column 8 of Table II, application no. 3, preferably a homolog or functional equivalent as depicted in column 8 of Table II B, application no. 3, and being depicted in the same respective line as said Ypr138c, and preferably the activity is increased non-targeted.

Accordingly, in one embodiment, the molecule which activity is to be increased in the process of the invention is the gene product with an activity as a “ammonium transporter”, preferably it is the molecule of section (a) or (b) of this paragraph.

In particular, it was observed that in plants, especially in Arabidopsis thaliana, increasing or generating the activity of a gene product non-targeted with the activity of a “ammonium transporter”, preferably being encoded by a gene comprising the nucleic acid sequence SEQ ID NO. 93218, preferably the coding region thereof, conferred the production of or the increase in aspartate compared with the wild type control.

The nucleic acid sequence of Zm_{—}4842_BE510522 from Zea mays, e.g. as shown in the respective line in column 5 of Table I, application no. 3, is unpublished. And the activity of the gene product thereof is the activity of Zm_{—}4842_BE510522-protein.

Accordingly, in one embodiment, the process of the present invention for producing phenylalanine in a non-human organism, like a microorganism or a plant or a part thereof, comprises increasing or generating the activity of a gene product with the activity of a gene product conferring the activity of “Zm_{—}4842_BE510522-protein”, especially from Zea mays or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Zm_{—}4842_BE510522, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Zm_{—}4842_BE510522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Zm_{—}4842_BE510522, or a functional equivalent or a homolog thereof as depicted in column 8 of Table II, application no. 3, preferably a homolog or functional equivalent as depicted in column 8 of Table II B, application no. 3, and being depicted in the same respective line as said Zm_{—}4842_BE510522, and preferably the activity is increased non-targeted.

Accordingly, in one embodiment, the molecule which activity is to be increased in the process of the invention is the gene product with an activity as a “Zm_{—}4842_BE510522-protein”, preferably it is the molecule of section (a) or (b) of this paragraph.

In particular, it was observed that in plants, especially in Arabidopsis thaliana, increasing or generating the activity of a gene product non-targeted with the activity of a “Zm_{—}4842_BE510522-protein”, preferably being encoded by a gene comprising the nucleic acid sequence SEQ ID NO. 68413, preferably the coding region thereof, conferred the production of or the increase in phenylalanine compared with the wild type control.

The nucleic acid sequence of Zm_{—}4842_BE510522 from Zea mays, e.g. as shown in the respective line in column 5 of Table I, application no. 3, is unpublished. And the activity of the gene product thereof is the activity of Zm_{—}4842_BE510522-protein.

Accordingly, in one embodiment, the process of the present invention for producing serine in a non-human organism, like a microorganism or a plant or a part thereof, comprises increasing or generating the activity of a gene product with the activity of a gene product conferring the activity of “Zm_{—}4842_BE510522-protein”, especially from Zea mays or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Zm_{—}4842_BE510522, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Zm_{—}4842_BE510522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Zm_{—}4842_BE510522, or a functional equivalent or a homolog thereof as depicted in column 8 of Table II, application no. 3, preferably a homolog or functional equivalent as depicted in column 8 of Table II B, application no. 3, and being depicted in the same respective line as said Zm_{—}4842_BE510522, and preferably the activity is increased non-targeted.

Accordingly, in one embodiment, the molecule which activity is to be increased in the process of the invention is the gene product with an activity as a “Zm_{—}4842_BE510522-protein”, preferably it is the molecule of section (a) or (b) of this paragraph.

In particular, it was observed that in plants, especially in Arabidopsis thaliana, increasing or generating the activity of a gene product non-targeted with the activity of a “Zm_{—}4842_BE510522-protein”, preferably being encoded by a gene comprising the nucleic acid sequence SEQ ID NO. 68413, preferably the coding region thereof, conferred the production of or the increase in serine compared with the wild type control.

The nucleic acid sequence of Zm_{—}4842_BE510522 from Zea mays, e.g. as shown in the respective line in column 5 of Table I, application no. 3, is unpublished. And the activity of the gene product thereof is the activity of Zm_{—}4842_BE510522-protein.

Accordingly, in one embodiment, the process of the present invention for producing tyrosine in a non-human organism, like a microorganism or a plant or a part thereof, comprises increasing or generating the activity of a gene product with the activity of a gene product conferring the activity of “Zm_{—}4842_BE510522-protein”, especially from Zea mays or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Zm_{—}4842_BE510522, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Zm_{—}4842_BE510522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Zm_{—}4842_BE510522, or a functional equivalent or a homolog thereof as depicted in column 8 of Table II, application no. 3, preferably a homolog or functional equivalent as depicted in column 8 of Table II B, application no. 3, and being depicted in the same respective line as said Zm_{—}4842_BE510522, and preferably the activity is increased non-targeted.

Accordingly, in one embodiment, the molecule which activity is to be increased in the process of the invention is the gene product with an activity as a “Zm_{—}4842_BE510522-protein”, preferably it is the molecule of section (a) or (b) of this paragraph.

In particular, it was observed that in plants, especially in Arabidopsis thaliana, increasing or generating the activity of a gene product non-targeted with the activity of a “Zm_{—}4842_BE510522-protein”, preferably being encoded by a gene comprising the nucleic acid sequence SEQ ID NO. 68413, preferably the coding region thereof, conferred the production of or the increase in tyrosine compared with the wild type control.

The nucleic acid sequence of Zm_{—}4842_BE510522 from Zea mays, e.g. as shown in the respective line in column 5 of Table I, application no. 3, is unpublished. And the activity of the gene product thereof is the activity of Zm_{—}4842_BE510522-protein.

Accordingly, in one embodiment, the process of the present invention for producing 5-oxoproline in a non-human organism, like a microorganism or a plant or a part thereof, comprises increasing or generating the activity of a gene product with the activity of a gene product conferring the activity of “Zm_{—}4842_BE510522-protein”, especially from Zea mays or its functional equivalent or its homolog, e.g. the increase of
(a) a gene product of a gene comprising the nucleic acid molecule as shown in the respective line in column 5 of Table I, application no. 3, preferably the coding region thereof, or a homolog or a fragment thereof, and being depicted in the same respective line as said Zm_{—}4842_BE510522, or a functional equivalent or a homolog thereof as shown in column 8 of Table I, application no. 3, preferably the coding region thereof, particularly a homolog or functional equivalent as shown in column 8 of Table I B, application no. 3, and being depicted in the same respective line as said Zm_{—}4842_BE510522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 3, respectively, and being depicted in the same respective line as said Zm_{—}4842_BE510522, or a functional equivalent or a homolog thereof as depicted in column 8 of Table II, application no. 3, preferably a homolog or functional equivalent as depicted in column 8 of Table II B, application no. 3, and being depicted in the same respective line as said Zm_{—}4842_BE510522, and preferably the activity is increased non-targeted.

Accordingly, in one embodiment, the molecule which activity is to be increased in the process of the invention is the gene product with an activity as a “Zm_{—}4842_BE510522-protein”, preferably it is the molecule of section (a) or (b) of this paragraph.

In particular, it was observed that in plants, especially in Arabidopsis thaliana, increasing or generating the activity of a gene product non-targeted with the activity of a “Zm_{—}4842_BE510522-protein”, preferably being encoded by a gene comprising the nucleic acid sequence SEQ ID NO. 68413, preferably the coding region thereof, conferred the production of or the increase in 5-oxoproline compared with the wild type control.

[0105.1.1.3] to [0107.1.1.3] for the disclosure of these paragraphs see [0105.1.1.1] to [0107.1.1.1] above.

A protein having an activity conferring an increase in the amount or level of the fine chemical 3,4-dihydroxyphenylalanine (dopa), 5-oxoproline, alanine, asparagine, aspartate, citrulline, glycine, homoserine, ornithine, phenylalanine, serine, or tyrosine, upon targeting to the plastids or mitochondria or upon non-targeting, preferably has the structure of the respective polypeptide described herein, in particular of the polypeptides comprising the consensus sequence or at least one polypeptide motifs as shown in the respective line in Table IV, application no. 3, column 8 or of the polypeptide comprising an amino acid sequence as disclosed in the respective line in Table II, application no. 3, columns 5 or 8, or homologs or fragments thereof as described herein, or is encoded by the nucleic acid molecule characterized herein or the nucleic acid molecule according to the invention, for example by the nucleic acid molecule as shown in the respective line in Table I, application no. 3, columns 5 or 8, preferably the coding region thereof, or homologs or fragments thereof and has the herein mentioned activity.

[0109.1.1.3] to [0110.1.1.3] for the disclosure of these paragraphs see [0109.1.1.1] to [0110.1.1.1] above.

In one embodiment, the process of the present invention comprises one or more of the following steps
(a) stabilizing a protein conferring the generation or increased expression of a protein encoded by the nucleic acid molecule of the invention or of the polypeptide of the invention, e.g. of a polypeptide having the activity selected from the group consisting of 2,3-dihydroxyphenylpropionate 1,2-dioxygenase, 2-C-methyl-D-erythritol 4-phosphate cytidylyltransferase, 2-oxoglutarate dehydrogenase E1 subunit, 30S ribosomal protein S3, 3deoxy-7-phosphoheptulonate synthase, 3-deoxy-D-arabinoheptulosonate-7-phosphatesynthase, 3-oxoacyl-[acyl-carrier-protein] synthase, 3-phosphoglycerate dehydrogenase, 47266012-protein, 49747384_SOYBEAN-protein, 50S ribosomal protein L14, 5′-nucleotidase, ABC metal ion transporter substrate-binding protein, ABC transporter ATP binding component, ABC transporter ATP-binding protein, ABC transporter component, ABC transporter permease protein, ABC transporter substrate-binding protein, acetolactate synthase, acetolactate synthase small subunit, acetyl CoA carboxylase, acetylCoA acetyltransferase, acetyltransferase, acid shock protein, acyl transferase, acyl-CoA dehydrogenase, acyl-CoA synthase, adenylate kinase, adenylylsulfate kinase, agmatinase, aldehyde dehydrogenase, amine oxidase, amino acid ABC transporter permease protein, amino acid acetyltransferase, amino acid transporter, amino-acid acetyltransferase, aminotransferase, ammonium transporter, ankyrin repeat family protein, anthranilate synthase component I, arginine exporter protein, arginine N-succinyltransferase, argininosuccinate lyase, asparaginase, aspartate 1-decarboxylase, aspartate aminotransferase, aspartate kinase, aspartate-ammonia ligase, At1g17440-protein, At1g19800-protein, At4g32480-protein, At5g16650-protein, at5g21910-protein, ATP-binding component of a transport system, ATP-dependent RNA helicase, auxin response factor, AX653549-protein, AY087308-protein, b0391-protein, b0456-protein, b0518-protein, b0801-protein, b0841-protein, b0917-protein, b1003-protein, b1024-protein, b1137-protein, b1155-protein, b1163-protein, b1179-protein, b1205-protein, b1229-protein, b1259-protein, b1280-protein, b1330-protein, b1445-protein, b1522-protein, b1583-protein, b1670-protein, b1672-protein, b1837-protein, b1898-protein, b1930-protein, b2032-protein, b2107-protein, b2360-protein, b2399-protein, b2474-protein, b2513-protein, b2613-protein, b2673-protein, b2739-protein, b2812-protein, b2849-protein, b3083-protein, b3098-protein, b3121-protein, b3221-protein, b3246-protein, b3346-protein, b3392-protein, b3410-protein, b3509-protein, b3646-protein, b3791-protein, b3814-protein, b3989-protein, b4029-protein, b4121-protein, beta-galactosidase, beta-hydroxylase, bifunctional aspartokinase/homoserine dehydrogenase, bifunctional purine biosynthesis protein, branched chain aminotransferase, branched-chain amino acid permease, calcium-dependent protein kinase, carbon dioxide concentrating mechanism protein, cation-transporting ATPase, CBL-interacting protein kinase, CDP-diacylglycerol-glycerol-3-phosphate 3-phosphatidyltransferase, CDP-diacylglycerol-serine O-phosphatidyltransferase, CDP-glucose-4,6-dehydratase, cell division control protein, cellulose synthase catalytic subunit, Chaperone protein CIpB, chorismate mutase-T and prephenate dehydrogenase, citrate synthase, coproporphyrinogen III oxidase, CTP synthetase, cullin, cyclin D, cystathionine gamma-synthase, cytochrome c-type protein, D-3-phosphoglycerate dehydrogenase, delta-1-pyrroline 5-carboxylase synthetase, diacylglycerol O-acyltransferase, dihydroxyacid dehydratase, DNA helicase II, DNA mismatch repair protein, DNA-binding protein, DnaJ-like chaperone, D-serine dehydratase, electron transfer flavoprotein subunit beta, elongation factor Tu, enoyl-CoA hydratase, enterobacterial common antigen polymerase, eukaryotic translation initiation factor, eukaryotic translation initiation factor 5, exopolyphosphatase, Fe—S subunit of oxidoreductase, flavodoxin, fumarylacetoacetate hydrolase, gamma-glutamyltranspeptidase, GDP-mannose dehydrogenase, geranylgeranyl pyrophosphate synthase, gibberellin 20-oxidase, Glu/Leu/Phe/Val dehydrogenase , glucokinase, glucose dehydrogenase, glucose-1-phosphate cytidylyltransferase, glucose-6-phosphate 1-dehydrogenase, glutamate-ammonia-ligase, glutamine amidotransferase, glutamine synthetase, glutaredoxin, glutathione peroxidase, glutathione S-transferase, Glycine cleavage system T aminomethyltransferase, glycogen (starch) synthase, glycogen synthase, glycoprotease, glycosyl transferase, GM02LC11114-protein, GM02LC17485-protein, harpin-induced family protein, heat shock transcription factor, HesB/YadR/YfhF family protein, histone acetyltransferase, histone H2A, homocitrate synthase, homoserine O-acetyltransferase, hydrolase, integral membrane transporter family protein, iron(III) dicitrate-binding protein, isochorismate synthase, kinase, L-aspartate oxidase, lipoprotein precursor, lysine decarboxylase, lysyl-tRNA synthetase, major facilitator superfamily transporter protein, malate dehydrogenase, malic enzyme, membrane protein, membrane transport protein, metal ion transporter, metal-dependent hydrolase, methylglyoxal synthase, methylglyoxalase, methyltransferase, mitochondrial inner membrane protease subunit, molecular chaperone portein, monothiol glutaredoxin, monthiol glutaredoxin, multidrug resistance protein, multiple antibiotic resistance protein, murein transglycosylase, N-(5′-phosphoribosyl) anthranilate isomerase, Na+/K+ transporter, N-acetyl-gamma-glutamyl-phosphate reductase, NADH dehydrogenase I chain I, NADH dehydrogenase subunit N, NADH-quinone oxidoreductase subunit, NADP-dependent malic enzyme, nitrate/nitrite transport protein, O-antigen chain length determinant, oxidoreductase, oxidoreductase subunit, oxireductase, peptidase, permease protein of phosphate ABC transporter, phosphate permease, phosphate transporter, phosphoadenosine phosphosulfate reductase , phosphoanhydride phosphorylase, phosphoenolpyruvate carboxykinase, phosphopantetheine adenylyltransferase, phosphoribosylformyl glycinamidine synthase subunit, Photosystem I reaction center subunit XI, photosystem II protein, polygalacturonase, precorrin methylase, Pre-mRNA-splicing factor, prolipoprotein diacylglyceryl transferase, protease, protein kinase, protein phosphatase, PTS system N-acetylmuramic acid-specific EIIBC component, purine biosynthesis protein, pyruvate kinase, restriction alleviation protein, riboflavin biosynthesis protein, RNA binding protein, RNA polymerase sigma factor, RNA-binding protein, s_pp015018333r-protein, sec-independent protein translocase, Sec-independent protein translocase subunit, serine acetyltransferase, serine hydroxymethyltransferase, serine protease, serine/threonine dehydratase, Serine/threonine-protein phosphatase, short-chain alcohol dehydrogenase family, sll0281-protein, sll1032-protein, sll1761-protein, sn-glycerol-3-phosphate dehydrogenase, sodium/proton antiporter, sterol 24-C-methyltransferase, thioredoxin, thioredoxin family protein, threonine dehydratase, threonine synthase, transcription factor, transcriptional regulator, transport protein, trehalose-phosphatase, tryptophan biosynthesis protein, tryptophan synthase alpha chain, TTC0768-protein, TTC0881-protein, TTC1386-protein, urease subunit, uridine/cytidine kinase, uroporphyrin-111 C-methyltransferase, XM_{—}473199-protein, ydr183w-protein, ydr273w-protein, ydr507c-protein, yer014w-protein, yer106w-protein, YFL019C-protein, yfl054c-protein, ygl096w-protein, ygl237c-protein, ygr221 c-protein, yhl013c-protein, yhr006w-protein, yhr207c-protein, YKL038W-protein, yml083c-protein, ymr013c-protein, YNL086W-protein, yol160w-protein, zinc finger protein, zinc transporter, and Zm_{—}4842_BE510522-protein, or of a polypeptide as indicated in the respective line in Table II, application no. 3, columns 5 or 8, or its homologs or fragments, and conferring the production of or an increase in 3,4-dihydroxyphenylalanine (dopa), 5-oxoproline, alanine, asparagine, aspartate, citrulline, glycine, homoserine, ornithine, phenylalanine, serine, or tyrosine, respectively; and/or
(b) stabilizing a mRNA conferring the generation or increased expression of a FCRP, e.g. protein encoded by the nucleic acid molecule of the invention or its homologs or fragments, or of a mRNA encoding the polypeptide of the present invention having the hereinmentioned activity selected from the group consisting of said activities mentioned in (a) and conferring the production of or an increase in 3,4-dihydroxyphenylalanine (dopa), 5-oxoproline, alanine, asparagine, aspartate, citrulline, glycine, homoserine, ornithine, phenylalanine, serine, or tyrosine, respectively; and/or
(c) increasing the specific activity of a protein conferring the increased expression of a FCRP, e.g. a protein encoded by the nucleic acid molecule of the invention or of the polypeptide of the present invention having herein-mentioned 3,4-dihydroxyphenylalanine (dopa), 5-oxoproline, alanine, asparagine, aspartate, citrulline, glycine, homoserine, ornithine, phenylalanine, serine, or tyrosine generating or increasing activity, respectively, e.g. of a polypeptide having the activity of a protein as indicated in the respective line in Table II, application no. 3, columns 5 or 8, or its homologs or fragments, or decreasing the inhibitory regulation of the polypeptide of the invention; and/or
(d) generating or increasing the expression of an endogenous or artificial transcription factor mediating the expression of a protein conferring the generation or increased expression of a FCRP, e.g. a protein encoded by the nucleic acid molecule of the invention or of the polypeptide of the invention having herein-mentioned activity selected from the group consisting of said activities mentioned in (a) and conferring a 3,4-dihydroxyphenylalanine (dopa), 5-oxoproline, alanine, asparagine, aspartate, citrulline, glycine, homoserine, ornithine, phenylalanine, serine, or tyrosine increasing activity, respectively, e.g. of a polypeptide having the activity of a protein as indicated in the respective line in Table II, application no. 3, columns 5 or 8 or its homologs; and/or
(e) stimulating activity of a protein conferring the increased expression of a FCRP, e.g. a protein encoded by the nucleic acid molecule of the present invention or a polypeptide of the present invention having herein-mentioned activity selected from the group consisting of said activities mentioned in (a) and conferring a 3,4-dihydroxyphenylalanine (dopa), 5-oxoproline, alanine, asparagine, aspartate, citrulline, glycine, homoserine, ornithine, phenylalanine, serine, or tyrosine increasing activity, respectively, e.g. of a polypeptide having the activity of a protein as indicated in the respective line in Table II, application no. 3, columns 5 or 8, or its homologs or fragments, by adding one or more exogenous inducing factors to the non-human organism or parts thereof; and/or
(f) expressing a transgenic gene encoding a protein conferring the increased expression of a FCRP, e.g. a polypeptide encoded by the nucleic acid molecule of the present invention or a polypeptide of the present invention, having herein-mentioned activity selected from the group consisting of said activities mentioned in (a) and conferring a 3,4-dihydroxyphenylalanine (dopa), 5-oxoproline, alanine, asparagine, aspartate, citrulline, glycine, homoserine, ornithine, phenylalanine, serine, or tyrosine increasing activity, respectively, e.g. of a polypeptide having the activity of a protein as indicated in the respective line in Table II, application no. 3, columns 5 or 8, or its homologs or fragments, and/or
(g) increasing the copy number of a gene conferring the increased expression of a nucleic acid molecule encoding a FCRP, e.g. a polypeptide encoded by the nucleic acid molecule of the invention or the polypeptide of the invention having herein-mentioned activity selected from the group consisting of said activities mentioned in (a) and conferring a 3,4-dihydroxyphenylalanine (dopa), 5-oxoproline, alanine, asparagine, aspartate, citrulline, glycine, homoserine, ornithine, phenylalanine, serine, or tyrosine increasing activity, respectively, e.g. of a polypeptide having the activity of a protein as indicated in the respective line in Table II, application no. 3, columns 5 or 8, or its homologs or fragments; and/or
(h) increasing the expression of the endogenous gene encoding the FCRP, polypeptide of the invention, e.g. a polypeptide encoded by the nucleic acid molecule of the invention or the polypeptide of the invention having herein-mentioned activity selected from the group consisting of said activities mentioned in (a) and conferring a 3,4-dihydroxyphenylalanine (dopa), 5-oxoproline, alanine, asparagine, aspartate, citrulline, glycine, homoserine, ornithine, phenylalanine, serine, or tyrosine; increasing activity, respectively, e.g. of a polypeptide having the activity of a protein as indicated in the respective line in Table II, application no. 3, columns 5 or 8, or its homologs or fragments, by adding positive expression or removing negative expression elements, e.g. homologous recombination can be used to either introduce positive regulatory elements like for plants the 35S enhancer into the promoter or to remove repressor elements form regulatory regions. Further gene conversion methods can be used to disrupt repressor elements or to enhance to activity of positive elements. Positive elements can be randomly introduced in plants by T-DNA or transposon mutagenesis and lines can be identified in which the positive elements have be integrated near to a gene of the invention, the expression of which is thereby enhanced; and/or
(i) modulating growth conditions of the non-human organism in such a manner, that the expression or activity of the gene encoding the FCRP, e.g. a protein of the invention or the protein itself is enhanced, for example microorganisms or plants can be grown for example under a higher temperature regime leading to an enhanced expression of heat shock proteins, which can lead to an enhanced 3,4-dihydroxyphenylalanine (dopa), 5-oxoproline, alanine, asparagine, aspartate, citrulline, glycine, homoserine, ornithine, phenylalanine, serine, or tyrosine production, respectively; and/or
(j) selecting of non-human organisms with especially high activity of the proteins of the invention from natural or from mutagenized resources and breeding them into the target organisms, e.g. the elite crops; and/or
(k) directing a protein encoded by the nucleic acid molecule of the invention or of the polypeptide of the present invention having herein-mentioned activity selected from the group consisting of said activities mentioned in (a) and conferring a 3,4-dihydroxyphenylalanine (dopa), 5-oxoproline, alanine, asparagine, aspartate, citrulline, glycine, homoserine, ornithine, phenylalanine, serine, or tyrosine increasing activity, respectively, e.g. of a polypeptide having the activity of a protein as indicated in the respective line in Table II, application no. 3, columns 5 or 8, or its homologs or fragments, if for the corresponding nucleic acid molecule in the respective line in column 6 of Table I the term “plastidic” is indicated, to the plastids by the addition of a plastidial targeting sequence or if for the corresponding nucleic acid molecule in the respective line in column 6 of Table I the term “mitochondria!” is indicated, to the mitochondria by the addition of a mitochondrial targeting sequence; and/or
(l) generating the expression of a protein encoded by the nucleic acid molecule of the invention or of the polypeptide of the present invention having herein-mentioned activity selected from the group consisting of said activities mentioned in (a) and conferring a 3,4-dihydroxyphenylalanine (dopa), 5-oxoproline, alanine, asparagine, aspartate, citrulline, glycine, homoserine, ornithine, phenylalanine, serine, or tyrosine increasing activity, respectively, e.g. of a polypeptide having the activity of a protein as indicated in the respective line in Table II, application no. 3, columns 5 or 8, or its homologs or fragments, if for the corresponding nucleic acid molecule in the respective line in column 6 of Table I the term “plastidic” or “mitochondrial” is indicated, in these organelles by the stable or transient transformation, advantageously stable transformation, of organelles, preferably plastids or mitochondria, with an inventive nucleic acid sequence preferably in form of an expression cassette containing said sequence leading to the expression of the nucleic acids or polypeptides of the invention in the respective organelle; and/or
(m) generating the expression of a protein encoded by the nucleic acid molecule of the invention or of the polypeptide of the present invention having herein-mentioned activity selected from the group consisting of said activities mentioned in (a) and conferring a 3,4-dihydroxyphenylalanine (dopa), 5-oxoproline, alanine, asparagine, aspartate, citrulline, glycine, homoserine, ornithine, phenylalanine, serine, and/or tyrosine increasing activity, respectively, e.g. of a polypeptide having the activity of a protein as indicated in the respective line in Table II, application no. 3, columns 5 or 8, or its homologs or fragments, if for the corresponding nucleic acid molecule in the respective line in column 6 of Table I the term “plastidic” or “mitochondrial” is indicated, in these organelles by integration of a nucleic acid of the invention into the genome of the respective organelle under control of preferable a promoter selective for the respective organelle.

Preferably, said mRNA is the coding region of the nucleic acid molecule of the present invention and/or the protein conferring the increased expression of a protein encoded by the coding region of the nucleic acid molecule of the present invention alone or linked to a transit nucleic acid sequence or transit peptide encoding nucleic acid sequence or the polypeptide having the herein mentioned activity, e.g. conferring the generation of or increase of 3,4-dihydroxyphenylalanine (dopa), 5-oxoproline, alanine, asparagine, aspartate, citrulline, glycine, homoserine, ornithine, phenylalanine, serine, or tyrosine, respectively, after increasing the expression or activity of the encoded polypeptide, non-targeted or in organelles such as plastids and/or mitochondria, preferably plastids, or having the activity of a polypeptide having an activity as the protein as shown in the respective line in Table II, application no. 3, column 3, or its homologs. Preferably the increase of 3,4-dihydroxyphenylalanine (dopa), 5-oxoproline, alanine, asparagine, aspartate, citrulline, glycine, homoserine, ornithine, phenylalanine, serine, or tyrosine, respectively, takes place non-targeted or in plastids and/or mitochondria, preferably non-targeted or in plastids.

[0113.1.1.3] to [0122.1.1.3] for the disclosure of these paragraphs see [0113.1.1.1] to [0122.1.1.1] above.

The activation of an endogenous polypeptide having above-mentioned activity, e.g. having the activity of a protein as shown in the respective line in Table II, application no. 3, column 3 or of the polypeptide of the invention, e.g. conferring the increase of the fine chemical 3,4-dihydroxyphenylalanine (dopa), 5-oxoproline, alanine, asparagine, aspartate, citrulline, glycine, homoserine, ornithine, phenylalanine, serine, or tyrosine, respectively, by increase of expression or activity in the cytoplasm, and/or in the cytosol, and/or in an organelle, such as plastids or mitochondria, can also be increased by introducing a synthetic transcription factor, which binds close to the coding region of the gene encoding the protein as shown in the respective line in Table II, application no. 3, column 5 or 8, or homologs or fragments thereof, and activates its transcription. A chimeric zinc finger protein can be constructed, which comprises a specific DNA-binding domain and an activation domain as e.g. the VP16 domain of Herpes Simplex virus. The specific binding domain can bind to the regulatory region of the gene encoding the protein as shown in the respective line in Table II, application no. 3, column 5 or 8, or homologs or fragments thereof. The expression of the chimeric transcription factor in a non-human organism, in particular in a plant, leads to a specific expression of the protein as shown in the respective line in Table II, application no. 3, column 5 or 8, or homologs or fragments thereof, see e.g. in WO01/52620, Oriz, Proc. Natl. Acad. Sci. USA 99, 13290 (2002), or Guan, Proc. Natl. Acad. Sci. USA 99, 13296 (2002).

[0124.1.1.3] to [0127.1.1.3] for the disclosure of these paragraphs see [0124.1.1.1] to [0127.1.1.1] above.

Owing to the introduction of a gene or a plurality of genes conferring the expression of the nucleic acid molecule of the invention or the polypeptide of the invention, for example the nucleic acid construct mentioned below, or encoding the protein as shown in the respective line in Table II, application no. 3, column 5 or 8, or homologs or fragments thereof, into a non-human organism alone or in combination with other genes, it is possible not only to increase the biosynthetic flux towards the end product, but also to increase, modify or create de novo an advantageous, preferably novel metabolites composition in the non-human organism, e.g. an advantageous composition comprising a higher content of (from a viewpoint of nutritional physiology limited) 3,4-dihydroxyphenylalanine (dopa), 5-oxoproline, alanine, asparagine, aspartate, citrulline, glycine, homoserine, ornithine, phenylalanine, serine, or tyrosine and if desired other amino acids, and/or other metabolites, in free or bound form.

for the disclosure of this paragraph see [0129.1.1.1] above.

Accordingly, in one embodiment, the process according to the invention relates to a process, which comprises:
(a) providing a non-human organism, preferably a microorganism, a plant cell, a plant tissue, a plant or a part thereof, more preferably a microorganism, a plant tissue, a plant or a part thereof;
(b) increasing the activity of a protein as shown in the respective line in Table II, application no. 3, column 3 or of a polypeptide being encoded by the respective nucleic acid molecule of the present invention and described below, e.g. conferring an increase of the fine chemical 3,4-dihydroxyphenylalanine (dopa), 5-oxoproline, alanine, asparagine, aspartate, citrulline, glycine, homoserine, ornithine, phenylalanine, serine, or tyrosine, respectively, in the non-human organism, preferably in the microorganism, the plant cell, the plant tissue, the plant or a part thereof, more preferably a microorganism, a plant tissue, a plant or a part thereof, especially cytoplasmic or in an organelle, like plastids or mitochondria,
(c) growing the non-human organism, preferably the microorganism, the plant cell, the plant tissue, the plant or a part thereof, under conditions which permit the production of said chemical in the non-human organism, preferably the microorganism, the plant cell, the plant tissue, the plant or a part thereof; and
(d) if desired, recovering, optionally isolating, said fine chemical, in free and/or bound form, and, optionally further free and/or bound amino acids, and/or other metabolites synthesized by the non-human organism, the microorganism, the plant cell, the plant tissue, the plant or a part thereof.

[0131.1.1.3] to [0140.1.3.3] for the disclosure of these paragraphs see [0131.1.1.1] to [0139.1.1.1] above.

In another embodiment, the invention relates to a (isolated) nucleic acid molecule, which comprises a nucleic acid molecule selected from the group consisting of:
(a) a nucleic acid molecule encoding a polypeptide shown in Table II, application no. 3, column 5 or 8, preferably shown in Table II B, application no. 3, column 8, or a homolog or a fragment thereof;
(b) a nucleic acid molecule shown in Table I, application no. 3, column 5 or 8, preferably shown in Table I B, application no. 3, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 3, preferably in column 8 of Table II B, application no. 3;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 3, preferably shown in column 8 of Table I B, application no. 3, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 3;
(i) a nucleic acid molecule encoding a polypeptide having the activity represented by a respective protein as depicted in column 5 of Table II, application no. 3;
(j) a nucleic acid molecule comprising a polynucleotide which is obtained by amplifying a cDNA library or a genomic library using the primers in Table III, application no. 3, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j); or a nucleic acid molecule comprising a sequence which is complementary thereto.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 3.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II application no. 3 and confers a generation or an increase of the respective fine chemical in a non-human organism or a part thereof as compared to a corresponding wild-type non-human organism or part thereof.

In another embodiment, the invention relates to a (isolated) nucleic acid molecule, which comprises a nucleic acid molecule selected from the group consisting of:
(a) a nucleic acid molecule encoding a polypeptide shown in Table II, column 5 or 8, application no. 3, preferably shown in Table II A, application no. 3, in column 5 or in Table II A, application no. 3, column 8 or in Table II B, application no. 3, column 8, or a homolog or a fragment thereof;
(b) a nucleic acid molecule shown in Table I, application no. 3, column 5 or 8, preferably shown in Table I A, application no. 3, in column 5 or in Table I A, application no. 3, column 8 or in Table I B, application no. 3, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 3, preferably shown in Table II A, application no. 3, in column 5 or in Table II A, application no. 3, column 8 or in Table II B, application no. 3, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 3, preferably shown in Table I A, application no. 3, in column 5 or in Table I A, application no. 3, column 8 or in Table I B column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 3;
(i) a nucleic acid molecule encoding a polypeptide having the activity represented by a respective protein as depicted in column 5 of Table II, application no. 3;
(j) a nucleic acid molecule comprising a polynucleotide which is obtained by amplifying a cDNA library or a genomic library using the primers in Table III, application no. 3, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt,500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
   or a nucleic acid molecule comprising a sequence which is complementary thereto;
   whereby, preferably, the nucleic acid molecule according to (a), (b), (c), (d), (e), (f), (g), (h), (i), (j) or (k) distinguishes over the sequence depicted in Table I A and/or I B, application no. 3, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 3, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 3, column 5 or 8, or the coding region thereof, respectively. In a further embodiment the nucleic acid molecule does not encode the polypeptide sequence shown in Table II A and/or II B, application no. 3, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 3, column 5 or 8 and does not encode a protein of the sequence shown in Table II A and/or II B, application no. 3, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 3, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 3, column 5 or 8 but less than 100%, preferably less than 99.999%, 99.99%, 99.9%, 99%, 98%, 97%, 96% or 95% identical to the sequence shown in Table II A and/or II B, application no. 3, column 5 or 8.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 3.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II, application no. 3, and confers a generation or an increase of the respective fine chemical in a non-human organism or a part thereof as compared to a corresponding wild-type non-human organism or part thereof.

[0144.1.1.3] to [0155.1.1.3] for the disclosure of these paragraphs see [0144.1.1.1] to [0155.1.1.1] above.

In a preferred embodiment a nucleic acid construct, for example an expression cassette, comprises upstream, i.e. at the 5′ end of the encoding sequence, a promoter and downstream, i.e. at the 3′ end, a polyadenylation signal and optionally other regulatory elements which are operably linked to the intervening encoding sequence with one of the nucleic acids as depicted in the respective line in Table I, application no. 3, column 5 or 8, preferably the coding region thereof, or a homolog or a fragment thereof. By an operable linkage is meant the sequential arrangement of promoter, encoding sequence, terminator, optionally other regulatory elements and optionally targeting sequences in such a way that each of the regulatory elements/targeting sequence can fulfill its function in the expression of the encoding sequence in due manner. In one embodiment the sequences preferred for operable linkage are targeting sequences for ensuring subcellular localization in organelles, like plastids or mitochondria. However, targeting sequences for ensuring subcellular localization in the endoplasmic reticulum (=ER), in the nucleus, in oil corpuscles or other compartments may also be employed as well as translation promoters such as the 5′ lead sequence in tobacco mosaic virus (Gallie et al., Nucl. Acids Res. 15, 8693 (1987)).

for the disclosure of this paragraph see [0157.1.1.1] above.

In an embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 3, column 5 or 8, preferably shown in Table II A, application no. 3, column 5, or in Table II A, application no. 3, column 8, or in Table II B, application no. 3, column 8, or a homolog or a fragment thereof;
   (b) a nucleic acid molecule shown in Table I, application no. 3, column 5 or 8, preferably shown in Table I A, application no. 3, column 5, or in Table I A, application no. 3, column 8, or in Table I B, application no. 3, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 3, preferably shown in Table II A, application no. 3, column 5, or in Table II A, application no. 3, column 8, or in Table II B, application no. 3, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 3, preferably shown in Table I A, application no. 3, column 5, or in Table I A, application no. 3, column 8, or in Table I B, application no. 3, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 3;
   (i) a nucleic acid molecule encoding a polypeptide having the activity represented by a respective protein as depicted in column 5 of Table II, application no. 3;
   (j) a nucleic acid molecule comprising a polynucleotide which is obtained by amplifying a cDNA library or a genomic library using the primers in Table III, application no. 3, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto.
      and, optionally
3) nucleic acid molecule encoding a transit peptide, preferably encoding a plastidal transit peptide or a mitochondrial transit peptide; which are operable linked.
   (All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 3.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 3 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 3, in the same line as of the respective nucleic acid molecule according to 2) “plastidic” is depicted, or encoding a mitochondrial transit peptide, especially in case in column 6 of Table I, application no. 3, in the same line as of the respective nucleic acid molecule according to 2) “mitochondrial” is depicted.

In another embodiment thereof the expression cassette does not comprise 3) a nucleic acid molecule encoding a transit peptide, especially in case in column 6 of Table I, application no. 3, in the same line as of the respective nucleic acid molecule according to 2) “non-targeted” is depicted.

In a further embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 3, column 5 or 8, preferably shown in Table II A, application no. 3, column 5, or in Table II A, application no. 3, column 8, or in Table II B, application no. 3, column 8, or a homolog or a fragment thereof;
   (b) a nucleic acid molecule shown in Table I, application no. 3, column 5 or 8, preferably shown in Table I A, application no. 3, column 5, or in Table I A, application no. 3, column 8, or in Table I B, application no. 3, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 3, preferably shown in Table II A, application no. 3, column 5, or in Table II A, application no. 3, column 8, or in Table II B, application no. 3, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 3, preferably shown in Table I A, application no. 3, column 5, or in Table I A, application no. 3, column 8,or in Table I B, application no. 3, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 3;
   (i) a nucleic acid molecule encoding a polypeptide having the activity represented by a respective protein as depicted in column 5 of Table II, application no. 3;
   (j) a nucleic acid molecule comprising a polynucleotide which is obtained by amplifying a cDNA library or a genomic library using the primers in Table III, application no. 3, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto; and, optionally
3) nucleic acid molecule encoding a transit peptide, preferably encoding a plastidal transit peptide or a mitochondrial transit peptide;
   which are operable linked and
   whereby, preferably, the nucleic acid molecule according to 2 (a), (b), (c), (d), (e), (f), (g), (h), (i), (j) or (k) distinguishes over the sequence depicted in Table I A and/or I B, application no. 3, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention according to 2) does not consist of the sequence shown in Table I A and/or I B, application no. 3, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 3, column 5 or 8, or the coding region thereof, respectively. In a further embodiment the nucleic acid molecule does not encode the polypeptide sequence shown in Table II A and/or II B, application no. 3, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 3, column 5 or 8 and does not encode a protein of the sequence shown in Table II A and/or II B, application no. 3, column 5 or 8. Accordingly, in one embodiment, the protein encoded by a sequence of a nucleic acid according to 2(a), (b), (c), (d), (e), (f), (g), (h), (i), (j) or (k) does not consist of the sequence shown in Table I A and/or I B, application no. 3, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 3, column 5 or 8 but less than 100%, 99.999%, 99.99%, 99.9%, 99%, 98%, 97%, 96% or 95% identical to the sequence shown in Table II A and/or II B, application no. 3, column 5 or 8.
   (All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 3.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 3 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 3, in the same line as of the respective nucleic acid molecule according to 2) “plastidic” is depicted, or encoding a mitochondrial transit peptide, especially in case in column 6 of Table I, application no. 3, in the same line as of the respective nucleic acid molecule according to 2) “mitochondrial” is depicted.

In another embodiment thereof the expression cassette does not comprise 3) a nucleic acid molecule encoding a transit peptide, especially in case in column 6 of Table I, application no. 3, in the same line as of the respective nucleic acid molecule according to 2) “non-targeted” is depicted.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 15187, or a homolog or fragment thereof, or a homolog thereof being depicted in Table I, application no. 3, column 8, in the same line as SEQ ID NO. 15187, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 15187 is depicted, plastidic is mentioned, or a mitochondrial transit peptide in case in Table I, application no. 3, in column 6 in the same line as SEQ ID NO. 15187 is depicted, mitochondrial is mentioned; which is operable linked, too.

In another embodiment thereof the expression cassette does not comprise an additional transit peptide in case in Table I, application no. 3, in column 6 in the same line as SEQ ID NO. 15187 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 69, or a homolog or fragment thereof, or a homolog thereof being depicted in Table I, application no. 3, column 8, in the same line as SEQ ID NO. 69, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 69 is depicted, plastidic is mentioned, or a mitochondrial transit peptide in case in Table I, application no. 3, in column 6 in the same line as SEQ ID NO. 69 is depicted, mitochondrial is mentioned; which is operable linked, too.

In another embodiment thereof the expression cassette does not comprise an additional transit peptide in case in Table I, application no. 3, in column 6 in the same line as SEQ ID NO. 69 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 15532, or a homolog or fragment thereof, or a homolog thereof being depicted in Table I, application no. 3, column 8, in the same line as SEQ ID NO. 15532, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 15532 is depicted, plastidic is mentioned, or a mitochondrial transit peptide in case in Table I, application no. 3, in column 6 in the same line as SEQ ID NO. 15532 is depicted, mitochondrial is mentioned; which is operable linked, too.

In another embodiment thereof the expression cassette does not comprise an additional transit peptide in case in Table I, application no. 3, in column 6 in the same line as SEQ ID NO. 15532 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 16155, or a homolog or fragment thereof, or a homolog thereof being depicted in Table I, application no. 3, column 8, in the same line as SEQ ID NO. 16155, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 16155 is depicted, plastidic is mentioned, or a mitochondrial transit peptide in case in Table I, application no. 3, in column 6 in the same line as SEQ ID NO. 16155 is depicted, mitochondrial is mentioned; which is operable linked, too.

In another embodiment thereof the expression cassette does not comprise an additional transit peptide in case in Table I, application no. 3, in column 6 in the same line as SEQ ID NO. 16155 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 16263, or a homolog or fragment thereof, or a homolog thereof being depicted in Table I, application no. 3, column 8, in the same line as SEQ ID NO. 16263, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 16263 is depicted, plastidic is mentioned, or a mitochondrial transit peptide in case in Table I, application no. 3, in column 6 in the same line as SEQ ID NO. 16263 is depicted, mitochondrial is mentioned; which is operable linked, too.

In another embodiment thereof the expression cassette does not comprise an additional transit peptide in case in Table I, application no. 3, in column 6 in the same line as SEQ ID NO. 16263 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 16883, or a homolog or fragment thereof, or a homolog thereof being depicted in Table I, application no. 3, column 8, in the same line as SEQ ID NO. 16883, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 16883 is depicted, plastidic is mentioned, or a mitochondrial transit peptide in case in Table I, application no. 3, in column 6 in the same line as SEQ ID NO. 16883 is depicted, mitochondrial is mentioned; which is operable linked, too.

In another embodiment thereof the expression cassette does not comprise an additional transit peptide in case in Table I, application no. 3, in column 6 in the same line as SEQ ID NO. 16883 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17356, or a homolog or fragment thereof, or a homolog thereof being depicted in Table I, application no. 3, column 8, in the same line as SEQ ID NO. 17356, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 17356 is depicted, plastidic is mentioned, or a mitochondrial transit peptide in case in Table I, application no. 3, in column 6 in the same line as SEQ ID NO. 17356 is depicted, mitochondrial is mentioned; which is operable linked, too.

In another embodiment thereof the expression cassette does not comprise an additional transit peptide in case in Table I, application no. 3, in column 6 in the same line as SEQ ID NO. 17356 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17451, or a homolog or fragment thereof, or a homolog thereof being depicted in Table I, application no. 3, column 8, in the same line as SEQ ID NO. 17451, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 17451 is depicted, plastidic is mentioned, or a mitochondrial transit peptide in case in Table I, application no. 3, in column 6 in the same line as SEQ ID NO. 17451 is depicted, mitochondrial is mentioned; which is operable linked, too.

In another embodiment thereof the expression cassette does not comprise an additional transit peptide in case in Table I, application no. 3, in column 6 in the same line as SEQ ID NO. 17451 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 385, or a homolog or fragment thereof, or a homolog thereof being depicted in Table I, application no. 3, column 8, in the same line as SEQ ID NO. 385, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 385 is depicted, plastidic is mentioned, or a mitochondrial transit peptide in case in Table I, application no. 3, in column 6 in the same line as SEQ ID NO. 385 is depicted, mitochondrial is mentioned; which is operable linked, too.

In another embodiment thereof the expression cassette does not comprise an additional transit peptide in case in Table I, application no. 3, in column 6 in the same line as SEQ ID NO. 385 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17601, or a homolog or fragment thereof, or a homolog thereof being depicted in Table I, application no. 3, column 8, in the same line as SEQ ID NO. 17601, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 17601 is depicted, plastidic is mentioned, or a mitochondrial transit peptide in case in Table I, application no. 3, in column 6 in the same line as SEQ ID NO. 17601 is depicted, mitochondrial is mentioned; which is operable linked, too.

In another embodiment thereof the expression cassette does not comprise an additional transit peptide in case in Table I, application no. 3, in column 6 in the same line as SEQ ID NO. 17601 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17637, or a homolog or fragment thereof, or a homolog thereof being depicted in Table I, application no. 3, column 8, in the same line as SEQ ID NO. 17637, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 17637 is depicted, plastidic is mentioned, or a mitochondrial transit peptide in case in Table I, application no. 3, in column 6 in the same line as SEQ ID NO. 17637 is depicted, mitochondrial is mentioned; which is operable linked, too.

In another embodiment thereof the expression cassette does not comprise an additional transit peptide in case in Table I, application no. 3, in column 6 in the same line as SEQ ID NO. 17637 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17701, or a homolog or fragment thereof, or a homolog thereof being depicted in Table I, application no. 3, column 8, in the same line as SEQ ID NO. 17701, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 17701 is depicted, plastidic is mentioned, or a mitochondrial transit peptide in case in Table I, application no. 3, in column 6 in the same line as SEQ ID NO. 17701 is depicted, mitochondrial is mentioned; which is operable linked, too.

In another embodiment thereof the expression cassette does not comprise an additional transit peptide in case in Table I, application no. 3, in column 6 in the same line as SEQ ID NO. 17701 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 627, or a homolog or fragment thereof, or a homolog thereof being depicted in Table I, application no. 3, column 8, in the same line as SEQ ID NO. 627, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 627 is depicted, plastidic is mentioned, or a mitochondrial transit peptide in case in Table I, application no. 3, in column 6 in the same line as SEQ ID NO. 627 is depicted, mitochondrial is mentioned; which is operable linked, too.

In another embodiment thereof the expression cassette does not comprise an additional transit peptide in case in Table I, application no. 3, in column 6 in the same line as SEQ ID NO. 627 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17968, or a homolog or fragment thereof, or a homolog thereof being depicted in Table I, application no. 3, column 8, in the same line as SEQ ID NO. 17968, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 17968 is depicted, plastidic is mentioned, or a mitochondrial transit peptide in case in Table I, application no. 3, in column 6 in the same line as SEQ ID NO. 17968 is depicted, mitochondrial is mentioned; which is operable linked, too.

In another embodiment thereof the expression cassette does not comprise an additional transit peptide in case in Table I, application no. 3, in column 6 in the same line as SEQ ID NO. 17968 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 18070, or a homolog or fragment thereof, or a homolog thereof being depicted in Table I, application no. 3, column 8, in the same line as SEQ ID NO. 18070, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 18070 is depicted, plastidic is mentioned, or a mitochondrial transit peptide in case in Table I, application no. 3, in column 6 in the same line as SEQ ID NO. 18070 is depicted, mitochondrial is mentioned; which is operable linked, too.

In another embodiment thereof the expression cassette does not comprise an additional transit peptide in case in Table I, application no. 3, in column 6 in the same line as SEQ ID NO. 18070 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 812, or a homolog or fragment thereof, or a homolog thereof being depicted in Table I, application no. 3, column 8, in the same line as SEQ ID NO. 812, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 812 is depicted, plastidic is mentioned, or a mitochondrial transit peptide in case in Table I, application no. 3, in column 6 in the same line as SEQ ID NO. 812 is depicted, mitochondrial is mentioned; which is operable linked, too.

In another embodiment thereof the expression cassette does not comprise an additional transit peptide in case in Table I, application no. 3, in column 6 in the same line as SEQ ID NO. 812 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 18235, or a homolog or fragment thereof, or a homolog thereof being depicted in Table I, application no. 3, column 8, in the same line as SEQ ID NO. 18235, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 18235 is depicted, plastidic is mentioned, or a mitochondrial transit peptide in case in Table I, application no. 3, in column 6 in the same line as SEQ ID NO. 18235 is depicted, mitochondrial is mentioned; which is operable linked, too.

In another embodiment thereof the expression cassette does not comprise an additional transit peptide in case in Table I, application no. 3, in column 6 in the same line as SEQ ID NO. 18235 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 18869, or a homolog or fragment thereof, or a homolog thereof being depicted in Table I, application no. 3, column 8, in the same line as SEQ ID NO. 18869, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 18869 is depicted, plastidic is mentioned, or a mitochondrial transit peptide in case in Table I, application no. 3, in column 6 in the same line as SEQ ID NO. 18869 is depicted, mitochondrial is mentioned; which is operable linked, too.

In another embodiment thereof the expression cassette does not comprise an additional transit peptide in case in Table I, application no. 3, in column 6 in the same line as SEQ ID NO. 18869 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 19419, or a homolog or fragment thereof, or a homolog thereof being depicted in Table I, application no. 3, column 8, in the same line as SEQ ID NO. 19419, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 19419 is depicted, plastidic is mentioned, or a mitochondrial transit peptide in case in Table I, application no. 3, in column 6 in the same line as SEQ ID NO. 19419 is depicted, mitochondrial is mentioned; which is operable linked, too.

In another embodiment thereof the expression cassette does not comprise an additional transit peptide in case in Table I, application no. 3, in column 6 in the same line as SEQ ID NO. 19419 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68536, or a homolog or fragment thereof, or a homolog thereof being depicted in Table I, application no. 3, column 8, in the same line as SEQ ID NO. 68536, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 68536 is depicted, plastidic is mentioned, or a mitochondrial transit peptide in case in Table I, application no. 3, in column 6 in the same line as SEQ ID NO. 68536 is depicted, mitochondrial is mentioned; which is operable linked, too.

In another embodiment thereof the expression cassette does not comprise an additional transit peptide in case in Table I, application no. 3, in column 6 in the same line as SEQ ID NO. 68536 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 19502, or a homolog or fragment thereof, or a homolog thereof being depicted in Table I, application no. 3, column 8, in the same line as SEQ ID NO. 19502, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 19502 is depicted, plastidic is mentioned, or a mitochondrial transit peptide in case in Table I, application no. 3, in column 6 in the same line as SEQ ID NO. 19502 is depicted, mitochondrial is mentioned; which is operable linked, too.

In another embodiment thereof the expression cassette does not comprise an additional transit peptide in case in Table I, application no. 3, in column 6 in the same line as SEQ ID NO. 19502 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 1298, or a homolog or fragment thereof, or a homolog thereof being depicted in Table I, application no. 3, column 8, in the same line as SEQ ID NO. 1298, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 1298 is depicted, plastidic is mentioned, or a mitochondrial transit peptide in case in Table I, application no. 3, in column 6 in the same line as SEQ ID NO. 1298 is depicted, mitochondrial is mentioned; which is operable linked, too.

In another embodiment thereof the expression cassette does not comprise an additional transit peptide in case in Table I, application no. 3, in column 6 in the same line as SEQ ID NO. 1298 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 19874, or a homolog or fragment thereof, or a homolog thereof being depicted in Table I, application no. 3, column 8, in the same line as SEQ ID NO. 19874, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 19874 is depicted, plastidic is mentioned, or a mitochondrial transit peptide in case in Table I, application no. 3, in column 6 in the same line as SEQ ID NO. 19874 is depicted, mitochondrial is mentioned; which is operable linked, too.

In another embodiment thereof the expression cassette does not comprise an additional transit peptide in case in Table I, application no. 3, in column 6 in the same line as SEQ ID NO. 19874 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 19919, or a homolog or fragment thereof, or a homolog thereof being depicted in Table I, application no. 3, column 8, in the same line as SEQ ID NO. 19919, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 19919 is depicted, plastidic is mentioned, or a mitochondrial transit peptide in case in Table I, application no. 3, in column 6 in the same line as SEQ ID NO. 19919 is depicted, mitochondrial is mentioned; which is operable linked, too.

In another embodiment thereof the expression cassette does not comprise an additional transit peptide in case in Table I, application no. 3, in column 6 in the same line as SEQ ID NO. 19919 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 20346, or a homolog or fragment thereof, or a homolog thereof being depicted in Table I, application no. 3, column 8, in the same line as SEQ ID NO. 20346, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 20346 is depicted, plastidic is mentioned, or a mitochondrial transit peptide in case in Table I, application no. 3, in column 6 in the same line as SEQ ID NO. 20346 is depicted, mitochondrial is mentioned; which is operable linked, too.

In another embodiment thereof the expression cassette does not comprise an additional transit peptide in case in Table I, application no. 3, in column 6 in the same line as SEQ ID NO. 20346 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 20578, or a homolog or fragment thereof, or a homolog thereof being depicted in Table I, application no. 3, column 8, in the same line as SEQ ID NO. 20578, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 20578 is depicted, plastidic is mentioned, or a mitochondrial transit peptide in case in Table I, application no. 3, in column 6 in the same line as SEQ ID NO. 20578 is depicted, mitochondrial is mentioned; which is operable linked, too.

In another embodiment thereof the expression cassette does not comprise an additional transit peptide in case in Table I, application no. 3, in column 6 in the same line as SEQ ID NO. 20578 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 21008, or a homolog or fragment thereof, or a homolog thereof being depicted in Table I, application no. 3, column 8, in the same line as SEQ ID NO. 21008, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 21008 is depicted, plastidic is mentioned, or a mitochondrial transit peptide in case in Table I, application no. 3, in column 6 in the same line as SEQ ID NO. 21008 is depicted, mitochondrial is mentioned; which is operable linked, too.

In another embodiment thereof the expression cassette does not comprise an additional transit peptide in case in Table I, application no. 3, in column 6 in the same line as SEQ ID NO. 21008 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 21159, or a homolog or fragment thereof, or a homolog thereof being depicted in Table I, application no. 3, column 8, in the same line as SEQ ID NO. 21159, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 21159 is depicted, plastidic is mentioned, or a mitochondrial transit peptide in case in Table I, application no. 3, in column 6 in the same line as SEQ ID NO. 21159 is depicted, mitochondrial is mentioned; which is operable linked, too.

In another embodiment thereof the expression cassette does not comprise an additional transit peptide in case in Table I, application no. 3, in column 6 in the same line as SEQ ID NO. 21159 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68657, or a homolog or fragment thereof, or a homolog thereof being depicted in Table I, application no. 3, column 8, in the same line as SEQ ID NO. 68657, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 68657 is depicted, plastidic is mentioned, or a mitochondrial transit peptide in case in Table I, application no. 3, in column 6 in the same line as SEQ ID NO. 68657 is depicted, mitochondrial is mentioned; which is operable linked, too.

In another embodiment thereof the expression cassette does not comprise an additional transit peptide in case in Table I, application no. 3, in column 6 in the same line as SEQ ID NO. 68657 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 21497, or a homolog or fragment thereof, or a homolog thereof being depicted in Table I, application no. 3, column 8, in the same line as SEQ ID NO. 21497, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 21497 is depicted, plastidic is mentioned, or a mitochondrial transit peptide in case in Table I, application no. 3, in column 6 in the same line as SEQ ID NO. 21497 is depicted, mitochondrial is mentioned; which is operable linked, too.

In another embodiment thereof the expression cassette does not comprise an additional transit peptide in case in Table I, application no. 3, in column 6 in the same line as SEQ ID NO. 21497 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 21902, or a homolog or fragment thereof, or a homolog thereof being depicted in Table I, application no. 3, column 8, in the same line as SEQ ID NO. 21902, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 21902 is depicted, plastidic is mentioned, or a mitochondrial transit peptide in case in Table I, application no. 3, in column 6 in the same line as SEQ ID NO. 21902 is depicted, mitochondrial is mentioned; which is operable linked, too.

In another embodiment thereof the expression cassette does not comprise an additional transit peptide in case in Table I, application no. 3, in column 6 in the same line as SEQ ID NO. 21902 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 22015, or a homolog or fragment thereof, or a homolog thereof being depicted in Table I, application no. 3, column 8, in the same line as SEQ ID NO. 22015, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 22015 is depicted, plastidic is mentioned, or a mitochondrial transit peptide in case in Table I, application no. 3, in column 6 in the same line as SEQ ID NO. 22015 is depicted, mitochondrial is mentioned; which is operable linked, too.

In another embodiment thereof the expression cassette does not comprise an additional transit peptide in case in Table I, application no. 3, in column 6 in the same line as SEQ ID NO. 22015 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 22249, or a homolog or fragment thereof, or a homolog thereof being depicted in Table I, application no. 3, column 8, in the same line as SEQ ID NO. 22249, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 22249 is depicted, plastidic is mentioned, or a mitochondrial transit peptide in case in Table I, application no. 3, in column 6 in the same line as SEQ ID NO. 22249 is depicted, mitochondrial is mentioned; which is operable linked, too.

In another embodiment thereof the expression cassette does not comprise an additional transit peptide in case in Table I, application no. 3, in column 6 in the same line as SEQ ID NO. 22249 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 22611, or a homolog or fragment thereof, or a homolog thereof being depicted in Table I, application no. 3, column 8, in the same line as SEQ ID NO. 22611, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 22611 is depicted, plastidic is mentioned, or a mitochondrial transit peptide in case in Table I, application no. 3, in column 6 in the same line as SEQ ID NO. 22611 is depicted, mitochondrial is mentioned; which is operable linked, too.

In another embodiment thereof the expression cassette does not comprise an additional transit peptide in case in Table I, application no. 3, in column 6 in the same line as SEQ ID NO. 22611 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 22832, or a homolog or fragment thereof, or a homolog thereof being depicted in Table I, application no. 3, column 8, in the same line as SEQ ID NO. 22832, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 22832 is depicted, plastidic is mentioned, or a mitochondrial transit peptide in case in Table I, application no. 3, in column 6 in the same line as SEQ ID NO. 22832 is depicted, mitochondrial is mentioned; which is operable linked, too.

In another embodiment thereof the expression cassette does not comprise an additional transit peptide in case in Table I, application no. 3, in column 6 in the same line as SEQ ID NO. 22832 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 1815, or a homolog or fragment thereof, or a homolog thereof being depicted in Table I, application no. 3, column 8, in the same line as SEQ ID NO. 1815, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 1815 is depicted, plastidic is mentioned, or a mitochondrial transit peptide in case in Table I, application no. 3, in column 6 in the same line as SEQ ID NO. 1815 is depicted, mitochondrial is mentioned; which is operable linked, too.

In another embodiment thereof the expression cassette does not comprise an additional transit peptide in case in Table I, application no. 3, in column 6 in the same line as SEQ ID NO. 1815 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68727, or a homolog or fragment thereof, or a homolog thereof being depicted in Table I, application no. 3, column 8, in the same line as SEQ ID NO. 68727, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 68727 is depicted, plastidic is mentioned, or a mitochondrial transit peptide in case in Table I, application no. 3, in column 6 in the same line as SEQ ID NO. 68727 is depicted, mitochondrial is mentioned; which is operable linked, too.

In another embodiment thereof the expression cassette does not comprise an additional transit peptide in case in Table I, application no. 3, in column 6 in the same line as SEQ ID NO. 68727 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 23002, or a homolog or fragment thereof, or a homolog thereof being depicted in Table I, application no. 3, column 8, in the same line as SEQ ID NO. 23002, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 23002 is depicted, plastidic is mentioned, or a mitochondrial transit peptide in case in Table I, application no. 3, in column 6 in the same line as SEQ ID NO. 23002 is depicted, mitochondrial is mentioned; which is operable linked, too.

In another embodiment thereof the expression cassette does not comprise an additional transit peptide in case in Table I, application no. 3, in column 6 in the same line as SEQ ID NO. 23002 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68777, or a homolog or fragment thereof, or a homolog thereof being depicted in Table I, application no. 3, column 8, in the same line as SEQ ID NO. 68777, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 68777 is depicted, plastidic is mentioned, or a mitochondrial transit peptide in case in Table I, application no. 3, in column 6 in the same line as SEQ ID NO. 68777 is depicted, mitochondrial is mentioned; which is operable linked, too.

In another embodiment thereof the expression cassette does not comprise an additional transit peptide in case in Table I, application no. 3, in column 6 in the same line as SEQ ID NO. 68777 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 2367, or a homolog or fragment thereof, or a homolog thereof being depicted in Table I, application no. 3, column 8, in the same line as SEQ ID NO. 2367, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 2367 is depicted, plastidic is mentioned, or a mitochondrial transit peptide in case in Table I, application no. 3, in column 6 in the same line as SEQ ID NO. 2367 is depicted, mitochondrial is mentioned; which is operable linked, too.

In another embodiment thereof the expression cassette does not comprise an additional transit peptide in case in Table I, application no. 3, in column 6 in the same line as SEQ ID NO. 2367 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 2573, or a homolog or fragment thereof, or a homolog thereof being depicted in Table I, application no. 3, column 8, in the same line as SEQ ID NO. 2573, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 2573 is depicted, plastidic is mentioned, or a mitochondrial transit peptide in case in Table I, application no. 3, in column 6 in the same line as SEQ ID NO. 2573 is depicted, mitochondrial is mentioned; which is operable linked, too.

In another embodiment thereof the expression cassette does not comprise an additional transit peptide in case in Table I, application no. 3, in column 6 in the same line as SEQ ID NO. 2573 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 2935, or a homolog or fragment thereof, or a homolog thereof being depicted in Table I, application no. 3, column 8, in the same line as SEQ ID NO. 2935, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 2935 is depicted, plastidic is mentioned, or a mitochondrial transit peptide in case in Table I, application no. 3, in column 6 in the same line as SEQ ID NO. 2935 is depicted, mitochondrial is mentioned; which is operable linked, too.

In another embodiment thereof the expression cassette does not comprise an additional transit peptide in case in Table I, application no. 3, in column 6 in the same line as SEQ ID NO. 2935 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 23482, or a homolog or fragment thereof, or a homolog thereof being depicted in Table I, application no. 3, column 8, in the same line as SEQ ID NO. 23482, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 23482 is depicted, plastidic is mentioned, or a mitochondrial transit peptide in case in Table I, application no. 3, in column 6 in the same line as SEQ ID NO. 23482 is depicted, mitochondrial is mentioned; which is operable linked, too.

In another embodiment thereof the expression cassette does not comprise an additional transit peptide in case in Table I, application no. 3, in column 6 in the same line as SEQ ID NO. 23482 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 3279, or a homolog or fragment thereof, or a homolog thereof being depicted in Table I, application no. 3, column 8, in the same line as SEQ ID NO. 3279, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 3279 is depicted, plastidic is mentioned, or a mitochondrial transit peptide in case in Table I, application no. 3, in column 6 in the same line as SEQ ID NO. 3279 is depicted, mitochondrial is mentioned; which is operable linked, too.

In another embodiment thereof the expression cassette does not comprise an additional transit peptide in case in Table I, application no. 3, in column 6 in the same line as SEQ ID NO. 3279 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 23844, or a homolog or fragment thereof, or a homolog thereof being depicted in Table I, application no. 3, column 8, in the same line as SEQ ID NO. 23844, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 23844 is depicted, plastidic is mentioned, or a mitochondrial transit peptide in case in Table I, application no. 3, in column 6 in the same line as SEQ ID NO. 23844 is depicted, mitochondrial is mentioned; which is operable linked, too.

In another embodiment thereof the expression cassette does not comprise an additional transit peptide in case in Table I, application no. 3, in column 6 in the same line as SEQ ID NO. 23844 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 3654, or a homolog or fragment thereof, or a homolog thereof being depicted in Table I, application no. 3, column 8, in the same line as SEQ ID NO. 3654, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 3654 is depicted, plastidic is mentioned, or a mitochondrial transit peptide in case in Table I, application no. 3, in column 6 in the same line as SEQ ID NO. 3654 is depicted, mitochondrial is mentioned; which is operable linked, too.

In another embodiment thereof the expression cassette does not comprise an additional transit peptide in case in Table I, application no. 3, in column 6 in the same line as SEQ ID NO. 3654 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 24232, or a homolog or fragment thereof, or a homolog thereof being depicted in Table I, application no. 3, column 8, in the same line as SEQ ID NO. 24232, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 24232 is depicted, plastidic is mentioned, or a mitochondrial transit peptide in case in Table I, application no. 3, in column 6 in the same line as SEQ ID NO. 24232 is depicted, mitochondrial is mentioned; which is operable linked, too.

In another embodiment thereof the expression cassette does not comprise an additional transit peptide in case in Table I, application no. 3, in column 6 in the same line as SEQ ID NO. 24232 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68849, or a homolog or fragment thereof, or a homolog thereof being depicted in Table I, application no. 3, column 8, in the same line as SEQ ID NO. 68849, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 68849 is depicted, plastidic is mentioned, or a mitochondrial transit peptide in case in Table I, application no. 3, in column 6 in the same line as SEQ ID NO. 68849 is depicted, mitochondrial is mentioned; which is operable linked, too.

In another embodiment thereof the expression cassette does not comprise an additional transit peptide in case in Table I, application no. 3, in column 6 in the same line as SEQ ID NO. 68849 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4040, or a homolog or fragment thereof, or a homolog thereof being depicted in Table I, application no. 3, column 8, in the same line as SEQ ID NO. 4040, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 4040 is depicted, plastidic is mentioned, or a mitochondrial transit peptide in case in Table I, application no. 3, in column 6 in the same line as SEQ ID NO. 4040 is depicted, mitochondrial is mentioned; which is operable linked, too.

In another embodiment thereof the expression cassette does not comprise an additional transit peptide in case in Table I, application no. 3, in column 6 in the same line as SEQ ID NO. 4040 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4102, or a homolog or fragment thereof, or a homolog thereof being depicted in Table I, application no. 3, column 8, in the same line as SEQ ID NO. 4102, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 4102 is depicted, plastidic is mentioned, or a mitochondrial transit peptide in case in Table I, application no. 3, in column 6 in the same line as SEQ ID NO. 4102 is depicted, mitochondrial is mentioned; which is operable linked, too.

In another embodiment thereof the expression cassette does not comprise an additional transit peptide in case in Table I, application no. 3, in column 6 in the same line as SEQ ID NO. 4102 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 24311, or a homolog or fragment thereof, or a homolog thereof being depicted in Table I, application no. 3, column 8, in the same line as SEQ ID NO. 24311, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 24311 is depicted, plastidic is mentioned, or a mitochondrial transit peptide in case in Table I, application no. 3, in column 6 in the same line as SEQ ID NO. 24311 is depicted, mitochondrial is mentioned; which is operable linked, too.

In another embodiment thereof the expression cassette does not comprise an additional transit peptide in case in Table I, application no. 3, in column 6 in the same line as SEQ ID NO. 24311 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4348, or a homolog or fragment thereof, or a homolog thereof being depicted in Table I, application no. 3, column 8, in the same line as SEQ ID NO. 4348, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 4348 is depicted, plastidic is mentioned, or a mitochondrial transit peptide in case in Table I, application no. 3, in column 6 in the same line as SEQ ID NO. 4348 is depicted, mitochondrial is mentioned; which is operable linked, too.

In another embodiment thereof the expression cassette does not comprise an additional transit peptide in case in Table I, application no. 3, in column 6 in the same line as SEQ ID NO. 4348 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68987, or a homolog or fragment thereof, or a homolog thereof being depicted in Table I, application no. 3, column 8, in the same line as SEQ ID NO. 68987, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 68987 is depicted, plastidic is mentioned, or a mitochondrial transit peptide in case in Table I, application no. 3, in column 6 in the same line as SEQ ID NO. 68987 is depicted, mitochondrial is mentioned; which is operable linked, too.

In another embodiment thereof the expression cassette does not comprise an additional transit peptide in case in Table I, application no. 3, in column 6 in the same line as SEQ ID NO. 68987 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 24438, or a homolog or fragment thereof, or a homolog thereof being depicted in Table I, application no. 3, column 8, in the same line as SEQ ID NO. 24438, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 24438 is depicted, plastidic is mentioned, or a mitochondrial transit peptide in case in Table I, application no. 3, in column 6 in the same line as SEQ ID NO. 24438 is depicted, mitochondrial is mentioned; which is operable linked, too.

In another embodiment thereof the expression cassette does not comprise an additional transit peptide in case in Table I, application no. 3, in column 6 in the same line as SEQ ID NO. 24438 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 24492, or a homolog or fragment thereof, or a homolog thereof being depicted in Table I, application no. 3, column 8, in the same line as SEQ ID NO. 24492, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 24492 is depicted, plastidic is mentioned, or a mitochondrial transit peptide in case in Table I, application no. 3, in column 6 in the same line as SEQ ID NO. 24492 is depicted, mitochondrial is mentioned; which is operable linked, too.

In another embodiment thereof the expression cassette does not comprise an additional transit peptide in case in Table I, application no. 3, in column 6 in the same line as SEQ ID NO. 24492 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 25222, or a homolog or fragment thereof, or a homolog thereof being depicted in Table I, application no. 3, column 8, in the same line as SEQ ID NO. 25222, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 25222 is depicted, plastidic is mentioned, or a mitochondrial transit peptide in case in Table I, application no. 3, in column 6 in the same line as SEQ ID NO. 25222 is depicted, mitochondrial is mentioned; which is operable linked, too.

In another embodiment thereof the expression cassette does not comprise an additional transit peptide in case in Table I, application no. 3, in column 6 in the same line as SEQ ID NO. 25222 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 69275, or a homolog or fragment thereof, or a homolog thereof being depicted in Table I, application no. 3, column 8, in the same line as SEQ ID NO. 69275, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 69275 is depicted, plastidic is mentioned, or a mitochondrial transit peptide in case in Table I, application no. 3, in column 6 in the same line as SEQ ID NO. 69275 is depicted, mitochondrial is mentioned; which is operable linked, too.

In another embodiment thereof the expression cassette does not comprise an additional transit peptide in case in Table I, application no. 3, in column 6 in the same line as SEQ ID NO. 69275 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 25283, or a homolog or fragment thereof, or a homolog thereof being depicted in Table I, application no. 3, column 8, in the same line as SEQ ID NO. 25283, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 25283 is depicted, plastidic is mentioned, or a mitochondrial transit peptide in case in Table I, application no. 3, in column 6 in the same line as SEQ ID NO. 25283 is depicted, mitochondrial is mentioned; which is operable linked, too.

In another embodiment thereof the expression cassette does not comprise an additional transit peptide in case in Table I, application no. 3, in column 6 in the same line as SEQ ID NO. 25283 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4904, or a homolog or fragment thereof, or a homolog thereof being depicted in Table I, application no. 3, column 8, in the same line as SEQ ID NO. 4904, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 4904 is depicted, plastidic is mentioned, or a mitochondrial transit peptide in case in Table I, application no. 3, in column 6 in the same line as SEQ ID NO. 4904 is depicted, mitochondrial is mentioned; which is operable linked, too.

In another embodiment thereof the expression cassette does not comprise an additional transit peptide in case in Table I, application no. 3, in column 6 in the same line as SEQ ID NO. 4904 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 69553, or a homolog or fragment thereof, or a homolog thereof being depicted in Table I, application no. 3, column 8, in the same line as SEQ ID NO. 69553, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 69553 is depicted, plastidic is mentioned, or a mitochondrial transit peptide in case in Table I, application no. 3, in column 6 in the same line as SEQ ID NO. 69553 is depicted, mitochondrial is mentioned; which is operable linked, too.

In another embodiment thereof the expression cassette does not comprise an additional transit peptide in case in Table I, application no. 3, in column 6 in the same line as SEQ ID NO. 69553 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 25428, or a homolog or fragment thereof, or a homolog thereof being depicted in Table I, application no. 3, column 8, in the same line as SEQ ID NO. 25428, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 25428 is depicted, plastidic is mentioned, or a mitochondrial transit peptide in case in Table I, application no. 3, in column 6 in the same line as SEQ ID NO. 25428 is depicted, mitochondrial is mentioned; which is operable linked, too.

In another embodiment thereof the expression cassette does not comprise an additional transit peptide in case in Table I, application no. 3, in column 6 in the same line as SEQ ID NO. 25428 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 69574, or a homolog or fragment thereof, or a homolog thereof being depicted in Table I, application no. 3, column 8, in the same line as SEQ ID NO. 69574, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 69574 is depicted, plastidic is mentioned, or a mitochondrial transit peptide in case in Table I, application no. 3, in column 6 in the same line as SEQ ID NO. 69574 is depicted, mitochondrial is mentioned; which is operable linked, too.

In another embodiment thereof the expression cassette does not comprise an additional transit peptide in case in Table I, application no. 3, in column 6 in the same line as SEQ ID NO. 69574 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 5318, or a homolog or fragment thereof, or a homolog thereof being depicted in Table I, application no. 3, column 8, in the same line as SEQ ID NO. 5318, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 5318 is depicted, plastidic is mentioned, or a mitochondrial transit peptide in case in Table I, application no. 3, in column 6 in the same line as SEQ ID NO. 5318 is depicted, mitochondrial is mentioned; which is operable linked, too.

In another embodiment thereof the expression cassette does not comprise an additional transit peptide in case in Table I, application no. 3, in column 6 in the same line as SEQ ID NO. 5318 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 25498, or a homolog or fragment thereof, or a homolog thereof being depicted in Table I, application no. 3, column 8, in the same line as SEQ ID NO. 25498, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 25498 is depicted, plastidic is mentioned, or a mitochondrial transit peptide in case in Table I, application no. 3, in column 6 in the same line as SEQ ID NO. 25498 is depicted, mitochondrial is mentioned; which is operable linked, too.

In another embodiment thereof the expression cassette does not comprise an additional transit peptide in case in Table I, application no. 3, in column 6 in the same line as SEQ ID NO. 25498 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 70006, or a homolog or fragment thereof, or a homolog thereof being depicted in Table I, application no. 3, column 8, in the same line as SEQ ID NO. 70006, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 70006 is depicted, plastidic is mentioned, or a mitochondrial transit peptide in case in Table I, application no. 3, in column 6 in the same line as SEQ ID NO. 70006 is depicted, mitochondrial is mentioned; which is operable linked, too.

In another embodiment thereof the expression cassette does not comprise an additional transit peptide in case in Table I, application no. 3, in column 6 in the same line as SEQ ID NO. 70006 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 70038, or a homolog or fragment thereof, or a homolog thereof being depicted in Table I, application no. 3, column 8, in the same line as SEQ ID NO. 70038, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 70038 is depicted, plastidic is mentioned, or a mitochondrial transit peptide in case in Table I, application no. 3, in column 6 in the same line as SEQ ID NO. 70038 is depicted, mitochondrial is mentioned; which is operable linked, too.

In another embodiment thereof the expression cassette does not comprise an additional transit peptide in case in Table I, application no. 3, in column 6 in the same line as SEQ ID NO. 70038 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 5493, or a homolog or fragment thereof, or a homolog thereof being depicted in Table I, application no. 3, column 8, in the same line as SEQ ID NO. 5493, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 5493 is depicted, plastidic is mentioned, or a mitochondrial transit peptide in case in Table I, application no. 3, in column 6 in the same line as SEQ ID NO. 5493 is depicted, mitochondrial is mentioned; which is operable linked, too.

In another embodiment thereof the expression cassette does not comprise an additional transit peptide in case in Table I, application no. 3, in column 6 in the same line as SEQ ID NO. 5493 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 70583, or a homolog or fragment thereof, or a homolog thereof being depicted in Table I, application no. 3, column 8, in the same line as SEQ ID NO. 70583, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 70583 is depicted, plastidic is mentioned, or a mitochondrial transit peptide in case in Table I, application no. 3, in column 6 in the same line as SEQ ID NO. 70583 is depicted, mitochondrial is mentioned; which is operable linked, too.

In another embodiment thereof the expression cassette does not comprise an additional transit peptide in case in Table I, application no. 3, in column 6 in the same line as SEQ ID NO. 70583 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 70715, or a homolog or fragment thereof, or a homolog thereof being depicted in Table I, application no. 3, column 8, in the same line as SEQ ID NO. 70715, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 70715 is depicted, plastidic is mentioned, or a mitochondrial transit peptide in case in Table I, application no. 3, in column 6 in the same line as SEQ ID NO. 70715 is depicted, mitochondrial is mentioned; which is operable linked, too.

In another embodiment thereof the expression cassette does not comprise an additional transit peptide in case in Table I, application no. 3, in column 6 in the same line as SEQ ID NO. 70715 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 25676, or a homolog or fragment thereof, or a homolog thereof being depicted in Table I, application no. 3, column 8, in the same line as SEQ ID NO. 25676, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 25676 is depicted, plastidic is mentioned, or a mitochondrial transit peptide in case in Table I, application no. 3, in column 6 in the same line as SEQ ID NO. 25676 is depicted, mitochondrial is mentioned; which is operable linked, too.

In another embodiment thereof the expression cassette does not comprise an additional transit peptide in case in Table I, application no. 3, in column 6 in the same line as SEQ ID NO. 25676 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 25780, or a homolog or fragment thereof, or a homolog thereof being depicted in Table I, application no. 3, column 8, in the same line as SEQ ID NO. 25780, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 25780 is depicted, plastidic is mentioned, or a mitochondrial transit peptide in case in Table I, application no. 3, in column 6 in the same line as SEQ ID NO. 25780 is depicted, mitochondrial is mentioned; which is operable linked, too.

In another embodiment thereof the expression cassette does not comprise an additional transit peptide in case in Table I, application no. 3, in column 6 in the same line as SEQ ID NO. 25780 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 5557, or a homolog or fragment thereof, or a homolog thereof being depicted in Table I, application no. 3, column 8, in the same line as SEQ ID NO. 5557, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 5557 is depicted, plastidic is mentioned, or a mitochondrial transit peptide in case in Table I, application no. 3, in column 6 in the same line as SEQ ID NO. 5557 is depicted, mitochondrial is mentioned; which is operable linked, too.

In another embodiment thereof the expression cassette does not comprise an additional transit peptide in case in Table I, application no. 3, in column 6 in the same line as SEQ ID NO. 5557 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 26196, or a homolog or fragment thereof, or a homolog thereof being depicted in Table I, application no. 3, column 8, in the same line as SEQ ID NO. 26196, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 26196 is depicted, plastidic is mentioned, or a mitochondrial transit peptide in case in Table I, application no. 3, in column 6 in the same line as SEQ ID NO. 26196 is depicted, mitochondrial is mentioned; which is operable linked, too.

In another embodiment thereof the expression cassette does not comprise an additional transit peptide in case in Table I, application no. 3, in column 6 in the same line as SEQ ID NO. 26196 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 26434, or a homolog or fragment thereof, or a homolog thereof being depicted in Table I, application no. 3, column 8, in the same line as SEQ ID NO. 26434, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 26434 is depicted, plastidic is mentioned, or a mitochondrial transit peptide in case in Table I, application no. 3, in column 6 in the same line as SEQ ID NO. 26434 is depicted, mitochondrial is mentioned; which is operable linked, too.

In another embodiment thereof the expression cassette does not comprise an additional transit peptide in case in Table I, application no. 3, in column 6 in the same line as SEQ ID NO. 26434 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6040, or a homolog or fragment thereof, or a homolog thereof being depicted in Table I, application no. 3, column 8, in the same line as SEQ ID NO. 6040, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 6040 is depicted, plastidic is mentioned, or a mitochondrial transit peptide in case in Table I, application no. 3, in column 6 in the same line as SEQ ID NO. 6040 is depicted, mitochondrial is mentioned; which is operable linked, too.

In another embodiment thereof the expression cassette does not comprise an additional transit peptide in case in Table I, application no. 3, in column 6 in the same line as SEQ ID NO. 6040 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 27021, or a homolog or fragment thereof, or a homolog thereof being depicted in Table I, application no. 3, column 8, in the same line as SEQ ID NO. 27021, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 27021 is depicted, plastidic is mentioned, or a mitochondrial transit peptide in case in Table I, application no. 3, in column 6 in the same line as SEQ ID NO. 27021 is depicted, mitochondrial is mentioned; which is operable linked, too.

In another embodiment thereof the expression cassette does not comprise an additional transit peptide in case in Table I, application no. 3, in column 6 in the same line as SEQ ID NO. 27021 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 71325, or a homolog or fragment thereof, or a homolog thereof being depicted in Table I, application no. 3, column 8, in the same line as SEQ ID NO. 71325, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 71325 is depicted, plastidic is mentioned, or a mitochondrial transit peptide in case in Table I, application no. 3, in column 6 in the same line as SEQ ID NO. 71325 is depicted, mitochondrial is mentioned; which is operable linked, too.

In another embodiment thereof the expression cassette does not comprise an additional transit peptide in case in Table I, application no. 3, in column 6 in the same line as SEQ ID NO. 71325 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 71763, or a homolog or fragment thereof, or a homolog thereof being depicted in Table I, application no. 3, column 8, in the same line as SEQ ID NO. 71763, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 71763 is depicted, plastidic is mentioned, or a mitochondrial transit peptide in case in Table I, application no. 3, in column 6 in the same line as SEQ ID NO. 71763 is depicted, mitochondrial is mentioned; which is operable linked, too.

In another embodiment thereof the expression cassette does not comprise an additional transit peptide in case in Table I, application no. 3, in column 6 in the same line as SEQ ID NO. 71763 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 27882, or a homolog or fragment thereof, or a homolog thereof being depicted in Table I, application no. 3, column 8, in the same line as SEQ ID NO. 27882, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 27882 is depicted, plastidic is mentioned, or a mitochondrial transit peptide in case in Table I, application no. 3, in column 6 in the same line as SEQ ID NO. 27882 is depicted, mitochondrial is mentioned; which is operable linked, too.

In another embodiment thereof the expression cassette does not comprise an additional transit peptide in case in Table I, application no. 3, in column 6 in the same line as SEQ ID NO. 27882 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 28040, or a homolog or fragment thereof, or a homolog thereof being depicted in Table I, application no. 3, column 8, in the same line as SEQ ID NO. 28040, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 28040 is depicted, plastidic is mentioned, or a mitochondrial transit peptide in case in Table I, application no. 3, in column 6 in the same line as SEQ ID NO. 28040 is depicted, mitochondrial is mentioned; which is operable linked, too.

In another embodiment thereof the expression cassette does not comprise an additional transit peptide in case in Table I, application no. 3, in column 6 in the same line as SEQ ID NO. 28040 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6075, or a homolog or fragment thereof, or a homolog thereof being depicted in Table I, application no. 3, column 8, in the same line as SEQ ID NO. 6075, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 6075 is depicted, plastidic is mentioned, or a mitochondrial transit peptide in case in Table I, application no. 3, in column 6 in the same line as SEQ ID NO. 6075 is depicted, mitochondrial is mentioned; which is operable linked, too.

In another embodiment thereof the expression cassette does not comprise an additional transit peptide in case in Table I, application no. 3, in column 6 in the same line as SEQ ID NO. 6075 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 72346, or a homolog or fragment thereof, or a homolog thereof being depicted in Table I, application no. 3, column 8, in the same line as SEQ ID NO. 72346, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 72346 is depicted, plastidic is mentioned, or a mitochondrial transit peptide in case in Table I, application no. 3, in column 6 in the same line as SEQ ID NO. 72346 is depicted, mitochondrial is mentioned; which is operable linked, too.

In another embodiment thereof the expression cassette does not comprise an additional transit peptide in case in Table I, application no. 3, in column 6 in the same line as SEQ ID NO. 72346 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 29246, or a homolog or fragment thereof, or a homolog thereof being depicted in Table I, application no. 3, column 8, in the same line as SEQ ID NO. 29246, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 29246 is depicted, plastidic is mentioned, or a mitochondrial transit peptide in case in Table I, application no. 3, in column 6 in the same line as SEQ ID NO. 29246 is depicted, mitochondrial is mentioned; which is operable linked, too.

In another embodiment thereof the expression cassette does not comprise an additional transit peptide in case in Table I, application no. 3, in column 6 in the same line as SEQ ID NO. 29246 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 29286, or a homolog or fragment thereof, or a homolog thereof being depicted in Table I, application no. 3, column 8, in the same line as SEQ ID NO. 29286, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 29286 is depicted, plastidic is mentioned, or a mitochondrial transit peptide in case in Table I, application no. 3, in column 6 in the same line as SEQ ID NO. 29286 is depicted, mitochondrial is mentioned; which is operable linked, too.

In another embodiment thereof the expression cassette does not comprise an additional transit peptide in case in Table I, application no. 3, in column 6 in the same line as SEQ ID NO. 29286 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 29500, or a homolog or fragment thereof, or a homolog thereof being depicted in Table I, application no. 3, column 8, in the same line as SEQ ID NO. 29500, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 29500 is depicted, plastidic is mentioned, or a mitochondrial transit peptide in case in Table I, application no. 3, in column 6 in the same line as SEQ ID NO. 29500 is depicted, mitochondrial is mentioned; which is operable linked, too.

In another embodiment thereof the expression cassette does not comprise an additional transit peptide in case in Table I, application no. 3, in column 6 in the same line as SEQ ID NO. 29500 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 30039, or a homolog or fragment thereof, or a homolog thereof being depicted in Table I, application no. 3, column 8, in the same line as SEQ ID NO. 30039, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 30039 is depicted, plastidic is mentioned, or a mitochondrial transit peptide in case in Table I, application no. 3, in column 6 in the same line as SEQ ID NO. 30039 is depicted, mitochondrial is mentioned; which is operable linked, too.

In another embodiment thereof the expression cassette does not comprise an additional transit peptide in case in Table I, application no. 3, in column 6 in the same line as SEQ ID NO. 30039 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 72520, or a homolog or fragment thereof, or a homolog thereof being depicted in Table I, application no. 3, column 8, in the same line as SEQ ID NO. 72520, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 72520 is depicted, plastidic is mentioned, or a mitochondrial transit peptide in case in Table I, application no. 3, in column 6 in the same line as SEQ ID NO. 72520 is depicted, mitochondrial is mentioned; which is operable linked, too.

In another embodiment thereof the expression cassette does not comprise an additional transit peptide in case in Table I, application no. 3, in column 6 in the same line as SEQ ID NO. 72520 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 31717, or a homolog or fragment thereof, or a homolog thereof being depicted in Table I, application no. 3, column 8, in the same line as SEQ ID NO. 31717, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 31717 is depicted, plastidic is mentioned, or a mitochondrial transit peptide in case in Table I, application no. 3, in column 6 in the same line as SEQ ID NO. 31717 is depicted, mitochondrial is mentioned; which is operable linked, too.

In another embodiment thereof the expression cassette does not comprise an additional transit peptide in case in Table I, application no. 3, in column 6 in the same line as SEQ ID NO. 31717 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 72935, or a homolog or fragment thereof, or a homolog thereof being depicted in Table I, application no. 3, column 8, in the same line as SEQ ID NO. 72935, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 72935 is depicted, plastidic is mentioned, or a mitochondrial transit peptide in case in Table I, application no. 3, in column 6 in the same line as SEQ ID NO. 72935 is depicted, mitochondrial is mentioned; which is operable linked, too.

In another embodiment thereof the expression cassette does not comprise an additional transit peptide in case in Table I, application no. 3, in column 6 in the same line as SEQ ID NO. 72935 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 73038, or a homolog or fragment thereof, or a homolog thereof being depicted in Table I, application no. 3, column 8, in the same line as SEQ ID NO. 73038, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 73038 is depicted, plastidic is mentioned, or a mitochondrial transit peptide in case in Table I, application no. 3, in column 6 in the same line as SEQ ID NO. 73038 is depicted, mitochondrial is mentioned; which is operable linked, too.

In another embodiment thereof the expression cassette does not comprise an additional transit peptide in case in Table I, application no. 3, in column 6 in the same line as SEQ ID NO. 73038 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 73284, or a homolog or fragment thereof, or a homolog thereof being depicted in Table I, application no. 3, column 8, in the same line as SEQ ID NO. 73284, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 73284 is depicted, plastidic is mentioned, or a mitochondrial transit peptide in case in Table I, application no. 3, in column 6 in the same line as SEQ ID NO. 73284 is depicted, mitochondrial is mentioned; which is operable linked, too.

In another embodiment thereof the expression cassette does not comprise an additional transit peptide in case in Table I, application no. 3, in column 6 in the same line as SEQ ID NO. 73284 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 31926, or a homolog or fragment thereof, or a homolog thereof being depicted in Table I, application no. 3, column 8, in the same line as SEQ ID NO. 31926, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 31926 is depicted, plastidic is mentioned, or a mitochondrial transit peptide in case in Table I, application no. 3, in column 6 in the same line as SEQ ID NO. 31926 is depicted, mitochondrial is mentioned; which is operable linked, too.

In another embodiment thereof the expression cassette does not comprise an additional transit peptide in case in Table I, application no. 3, in column 6 in the same line as SEQ ID NO. 31926 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 73719, or a homolog or fragment thereof, or a homolog thereof being depicted in Table I, application no. 3, column 8, in the same line as SEQ ID NO. 73719, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 73719 is depicted, plastidic is mentioned, or a mitochondrial transit peptide in case in Table I, application no. 3, in column 6 in the same line as SEQ ID NO. 73719 is depicted, mitochondrial is mentioned; which is operable linked, too.

In another embodiment thereof the expression cassette does not comprise an additional transit peptide in case in Table I, application no. 3, in column 6 in the same line as SEQ ID NO. 73719 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 74163, or a homolog or fragment thereof, or a homolog thereof being depicted in Table I, application no. 3, column 8, in the same line as SEQ ID NO. 74163, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 74163 is depicted, plastidic is mentioned, or a mitochondrial transit peptide in case in Table I, application no. 3, in column 6 in the same line as SEQ ID NO. 74163 is depicted, mitochondrial is mentioned; which is operable linked, too.

In another embodiment thereof the expression cassette does not comprise an additional transit peptide in case in Table I, application no. 3, in column 6 in the same line as SEQ ID NO. 74163 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 74452, or a homolog or fragment thereof, or a homolog thereof being depicted in Table I, application no. 3, column 8, in the same line as SEQ ID NO. 74452, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 74452 is depicted, plastidic is mentioned, or a mitochondrial transit peptide in case in Table I, application no. 3, in column 6 in the same line as SEQ ID NO. 74452 is depicted, mitochondrial is mentioned; which is operable linked, too.

In another embodiment thereof the expression cassette does not comprise an additional transit peptide in case in Table I, application no. 3, in column 6 in the same line as SEQ ID NO. 74452 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6510, or a homolog or fragment thereof, or a homolog thereof being depicted in Table I, application no. 3, column 8, in the same line as SEQ ID NO. 6510, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 6510 is depicted, plastidic is mentioned, or a mitochondrial transit peptide in case in Table I, application no. 3, in column 6 in the same line as SEQ ID NO. 6510 is depicted, mitochondrial is mentioned; which is operable linked, too.

In another embodiment thereof the expression cassette does not comprise an additional transit peptide in case in Table I, application no. 3, in column 6 in the same line as SEQ ID NO. 6510 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 32037, or a homolog or fragment thereof, or a homolog thereof being depicted in Table I, application no. 3, column 8, in the same line as SEQ ID NO. 32037, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 32037 is depicted, plastidic is mentioned, or a mitochondrial transit peptide in case in Table I, application no. 3, in column 6 in the same line as SEQ ID NO. 32037 is depicted, mitochondrial is mentioned; which is operable linked, too.

In another embodiment thereof the expression cassette does not comprise an additional transit peptide in case in Table I, application no. 3, in column 6 in the same line as SEQ ID NO. 32037 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 32308, or a homolog or fragment thereof, or a homolog thereof being depicted in Table I, application no. 3, column 8, in the same line as SEQ ID NO. 32308, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 32308 is depicted, plastidic is mentioned, or a mitochondrial transit peptide in case in Table I, application no. 3, in column 6 in the same line as SEQ ID NO. 32308 is depicted, mitochondrial is mentioned; which is operable linked, too.

In another embodiment thereof the expression cassette does not comprise an additional transit peptide in case in Table I, application no. 3, in column 6 in the same line as SEQ ID NO. 32308 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 32648, or a homolog or fragment thereof, or a homolog thereof being depicted in Table I, application no. 3, column 8, in the same line as SEQ ID NO. 32648, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 32648 is depicted, plastidic is mentioned, or a mitochondrial transit peptide in case in Table I, application no. 3, in column 6 in the same line as SEQ ID NO. 32648 is depicted, mitochondrial is mentioned; which is operable linked, too.

In another embodiment thereof the expression cassette does not comprise an additional transit peptide in case in Table I, application no. 3, in column 6 in the same line as SEQ ID NO. 32648 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 74662, or a homolog or fragment thereof, or a homolog thereof being depicted in Table I, application no. 3, column 8, in the same line as SEQ ID NO. 74662, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 74662 is depicted, plastidic is mentioned, or a mitochondrial transit peptide in case in Table I, application no. 3, in column 6 in the same line as SEQ ID NO. 74662 is depicted, mitochondrial is mentioned; which is operable linked, too.

In another embodiment thereof the expression cassette does not comprise an additional transit peptide in case in Table I, application no. 3, in column 6 in the same line as SEQ ID NO. 74662 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 33085, or a homolog or fragment thereof, or a homolog thereof being depicted in Table I, application no. 3, column 8, in the same line as SEQ ID NO. 33085, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 33085 is depicted, plastidic is mentioned, or a mitochondrial transit peptide in case in Table I, application no. 3, in column 6 in the same line as SEQ ID NO. 33085 is depicted, mitochondrial is mentioned; which is operable linked, too.

In another embodiment thereof the expression cassette does not comprise an additional transit peptide in case in Table I, application no. 3, in column 6 in the same line as SEQ ID NO. 33085 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 33596, or a homolog or fragment thereof, or a homolog thereof being depicted in Table I, application no. 3, column 8, in the same line as SEQ ID NO. 33596, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 33596 is depicted, plastidic is mentioned, or a mitochondrial transit peptide in case in Table I, application no. 3, in column 6 in the same line as SEQ ID NO. 33596 is depicted, mitochondrial is mentioned; which is operable linked, too.

In another embodiment thereof the expression cassette does not comprise an additional transit peptide in case in Table I, application no. 3, in column 6 in the same line as SEQ ID NO. 33596 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 74729, or a homolog or fragment thereof, or a homolog thereof being depicted in Table I, application no. 3, column 8, in the same line as SEQ ID NO. 74729, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 74729 is depicted, plastidic is mentioned, or a mitochondrial transit peptide in case in Table I, application no. 3, in column 6 in the same line as SEQ ID NO. 74729 is depicted, mitochondrial is mentioned; which is operable linked, too.

In another embodiment thereof the expression cassette does not comprise an additional transit peptide in case in Table I, application no. 3, in column 6 in the same line as SEQ ID NO. 74729 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 34301, or a homolog or fragment thereof, or a homolog thereof being depicted in Table I, application no. 3, column 8, in the same line as SEQ ID NO. 34301, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 34301 is depicted, plastidic is mentioned, or a mitochondrial transit peptide in case in Table I, application no. 3, in column 6 in the same line as SEQ ID NO. 34301 is depicted, mitochondrial is mentioned; which is operable linked, too.

In another embodiment thereof the expression cassette does not comprise an additional transit peptide in case in Table I, application no. 3, in column 6 in the same line as SEQ ID NO. 34301 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 34602, or a homolog or fragment thereof, or a homolog thereof being depicted in Table I, application no. 3, column 8, in the same line as SEQ ID NO. 34602, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 34602 is depicted, plastidic is mentioned, or a mitochondrial transit peptide in case in Table I, application no. 3, in column 6 in the same line as SEQ ID NO. 34602 is depicted, mitochondrial is mentioned; which is operable linked, too.

In another embodiment thereof the expression cassette does not comprise an additional transit peptide in case in Table I, application no. 3, in column 6 in the same line as SEQ ID NO. 34602 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 34889, or a homolog or fragment thereof, or a homolog thereof being depicted in Table I, application no. 3, column 8, in the same line as SEQ ID NO. 34889, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 34889 is depicted, plastidic is mentioned, or a mitochondrial transit peptide in case in Table I, application no. 3, in column 6 in the same line as SEQ ID NO. 34889 is depicted, mitochondrial is mentioned; which is operable linked, too.

In another embodiment thereof the expression cassette does not comprise an additional transit peptide in case in Table I, application no. 3, in column 6 in the same line as SEQ ID NO. 34889 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 6818, or a homolog or fragment thereof, or a homolog thereof being depicted in Table I, application no. 3, column 8, in the same line as SEQ ID NO. 6818, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 6818 is depicted, plastidic is mentioned, or a mitochondrial transit peptide in case in Table I, application no. 3, in column 6 in the same line as SEQ ID NO. 6818 is depicted, mitochondrial is mentioned; which is operable linked, too.

In another embodiment thereof the expression cassette does not comprise an additional transit peptide in case in Table I, application no. 3, in column 6 in the same line as SEQ ID NO. 6818 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 35482, or a homolog or fragment thereof, or a homolog thereof being depicted in Table I, application no. 3, column 8, in the same line as SEQ ID NO. 35482, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 35482 is depicted, plastidic is mentioned, or a mitochondrial transit peptide in case in Table I, application no. 3, in column 6 in the same line as SEQ ID NO. 35482 is depicted, mitochondrial is mentioned; which is operable linked, too.

In another embodiment thereof the expression cassette does not comprise an additional transit peptide in case in Table I, application no. 3, in column 6 in the same line as SEQ ID NO. 35482 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 35482, or a homolog or fragment thereof, or a homolog thereof being depicted in Table I, application no. 3, column 8, in the same line as SEQ ID NO. 35482, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 35482 is depicted, plastidic is mentioned, or a mitochondrial transit peptide in case in Table I, application no. 3, in column 6 in the same line as SEQ ID NO. 35482 is depicted, mitochondrial is mentioned; which is operable linked, too.

In another embodiment thereof the expression cassette does not comprise an additional transit peptide in case in Table I, application no. 3, in column 6 in the same line as SEQ ID NO. 35482 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 74986, or a homolog or fragment thereof, or a homolog thereof being depicted in Table I, application no. 3, column 8, in the same line as SEQ ID NO. 74986, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 74986 is depicted, plastidic is mentioned, or a mitochondrial transit peptide in case in Table I, application no. 3, in column 6 in the same line as SEQ ID NO. 74986 is depicted, mitochondrial is mentioned; which is operable linked, too.

In another embodiment thereof the expression cassette does not comprise an additional transit peptide in case in Table I, application no. 3, in column 6 in the same line as SEQ ID NO. 74986 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7081, or a homolog or fragment thereof, or a homolog thereof being depicted in Table I, application no. 3, column 8, in the same line as SEQ ID NO. 7081, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 7081 is depicted, plastidic is mentioned, or a mitochondrial transit peptide in case in Table I, application no. 3, in column 6 in the same line as SEQ ID NO. 7081 is depicted, mitochondrial is mentioned; which is operable linked, too.

In another embodiment thereof the expression cassette does not comprise an additional transit peptide in case in Table I, application no. 3, in column 6 in the same line as SEQ ID NO. 7081 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 35590, or a homolog or fragment thereof, or a homolog thereof being depicted in Table I, application no. 3, column 8, in the same line as SEQ ID NO. 35590, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 35590 is depicted, plastidic is mentioned, or a mitochondrial transit peptide in case in Table I, application no. 3, in column 6 in the same line as SEQ ID NO. 35590 is depicted, mitochondrial is mentioned; which is operable linked, too.

In another embodiment thereof the expression cassette does not comprise an additional transit peptide in case in Table I, application no. 3, in column 6 in the same line as SEQ ID NO. 35590 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 35733, or a homolog or fragment thereof, or a homolog thereof being depicted in Table I, application no. 3, column 8, in the same line as SEQ ID NO. 35733, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 35733 is depicted, plastidic is mentioned, or a mitochondrial transit peptide in case in Table I, application no. 3, in column 6 in the same line as SEQ ID NO. 35733 is depicted, mitochondrial is mentioned; which is operable linked, too.

In another embodiment thereof the expression cassette does not comprise an additional transit peptide in case in Table I, application no. 3, in column 6 in the same line as SEQ ID NO. 35733 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 7269, or a homolog or fragment thereof, or a homolog thereof being depicted in Table I, application no. 3, column 8, in the same line as SEQ ID NO. 7269, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 7269 is depicted, plastidic is mentioned, or a mitochondrial transit peptide in case in Table I, application no. 3, in column 6 in the same line as SEQ ID NO. 7269 is depicted, mitochondrial is mentioned; which is operable linked, too.

In another embodiment thereof the expression cassette does not comprise an additional transit peptide in case in Table I, application no. 3, in column 6 in the same line as SEQ ID NO. 7269 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 75195, or a homolog or fragment thereof, or a homolog thereof being depicted in Table I, application no. 3, column 8, in the same line as SEQ ID NO. 75195, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 75195 is depicted, plastidic is mentioned, or a mitochondrial transit peptide in case in Table I, application no. 3, in column 6 in the same line as SEQ ID NO. 75195 is depicted, mitochondrial is mentioned; which is operable linked, too.

In another embodiment thereof the expression cassette does not comprise an additional transit peptide in case in Table I, application no. 3, in column 6 in the same line as SEQ ID NO. 75195 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7333, or a homolog or fragment thereof, or a homolog thereof being depicted in Table I, application no. 3, column 8, in the same line as SEQ ID NO. 7333, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 7333 is depicted, plastidic is mentioned, or a mitochondrial transit peptide in case in Table I, application no. 3, in column 6 in the same line as SEQ ID NO. 7333 is depicted, mitochondrial is mentioned; which is operable linked, too.

In another embodiment thereof the expression cassette does not comprise an additional transit peptide in case in Table I, application no. 3, in column 6 in the same line as SEQ ID NO. 7333 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 35875, or a homolog or fragment thereof, or a homolog thereof being depicted in Table I, application no. 3, column 8, in the same line as SEQ ID NO. 35875, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 35875 is depicted, plastidic is mentioned, or a mitochondrial transit peptide in case in Table I, application no. 3, in column 6 in the same line as SEQ ID NO. 35875 is depicted, mitochondrial is mentioned; which is operable linked, too.

In another embodiment thereof the expression cassette does not comprise an additional transit peptide in case in Table I, application no. 3, in column 6 in the same line as SEQ ID NO. 35875 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7686, or a homolog or fragment thereof, or a homolog thereof being depicted in Table I, application no. 3, column 8, in the same line as SEQ ID NO. 7686, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 7686 is depicted, plastidic is mentioned, or a mitochondrial transit peptide in case in Table I, application no. 3, in column 6 in the same line as SEQ ID NO. 7686 is depicted, mitochondrial is mentioned; which is operable linked, too.

In another embodiment thereof the expression cassette does not comprise an additional transit peptide in case in Table I, application no. 3, in column 6 in the same line as SEQ ID NO. 7686 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 35936, or a homolog or fragment thereof, or a homolog thereof being depicted in Table I, application no. 3, column 8, in the same line as SEQ ID NO. 35936, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 35936 is depicted, plastidic is mentioned, or a mitochondrial transit peptide in case in Table I, application no. 3, in column 6 in the same line as SEQ ID NO. 35936 is depicted, mitochondrial is mentioned; which is operable linked, too.

In another embodiment thereof the expression cassette does not comprise an additional transit peptide in case in Table I, application no. 3, in column 6 in the same line as SEQ ID NO. 35936 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 35967, or a homolog or fragment thereof, or a homolog thereof being depicted in Table I, application no. 3, column 8, in the same line as SEQ ID NO. 35967, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 35967 is depicted, plastidic is mentioned, or a mitochondrial transit peptide in case in Table I, application no. 3, in column 6 in the same line as SEQ ID NO. 35967 is depicted, mitochondrial is mentioned; which is operable linked, too.

In another embodiment thereof the expression cassette does not comprise an additional transit peptide in case in Table I, application no. 3, in column 6 in the same line as SEQ ID NO. 35967 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 36114, or a homolog or fragment thereof, or a homolog thereof being depicted in Table I, application no. 3, column 8, in the same line as SEQ ID NO. 36114, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 36114 is depicted, plastidic is mentioned, or a mitochondrial transit peptide in case in Table I, application no. 3, in column 6 in the same line as SEQ ID NO. 36114 is depicted, mitochondrial is mentioned; which is operable linked, too.

In another embodiment thereof the expression cassette does not comprise an additional transit peptide in case in Table I, application no. 3, in column 6 in the same line as SEQ ID NO. 36114 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 75286, or a homolog or fragment thereof, or a homolog thereof being depicted in Table I, application no. 3, column 8, in the same line as SEQ ID NO. 75286, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 75286 is depicted, plastidic is mentioned, or a mitochondrial transit peptide in case in Table I, application no. 3, in column 6 in the same line as SEQ ID NO. 75286 is depicted, mitochondrial is mentioned; which is operable linked, too.

In another embodiment thereof the expression cassette does not comprise an additional transit peptide in case in Table I, application no. 3, in column 6 in the same line as SEQ ID NO. 75286 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 75807, or a homolog or fragment thereof, or a homolog thereof being depicted in Table I, application no. 3, column 8, in the same line as SEQ ID NO. 75807, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 75807 is depicted, plastidic is mentioned, or a mitochondrial transit peptide in case in Table I, application no. 3, in column 6 in the same line as SEQ ID NO. 75807 is depicted, mitochondrial is mentioned; which is operable linked, too.

In another embodiment thereof the expression cassette does not comprise an additional transit peptide in case in Table I, application no. 3, in column 6 in the same line as SEQ ID NO. 75807 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 36299, or a homolog or fragment thereof, or a homolog thereof being depicted in Table I, application no. 3, column 8, in the same line as SEQ ID NO. 36299, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 36299 is depicted, plastidic is mentioned, or a mitochondrial transit peptide in case in Table I, application no. 3, in column 6 in the same line as SEQ ID NO. 36299 is depicted, mitochondrial is mentioned; which is operable linked, too.

In another embodiment thereof the expression cassette does not comprise an additional transit peptide in case in Table I, application no. 3, in column 6 in the same line as SEQ ID NO. 36299 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 75873, or a homolog or fragment thereof, or a homolog thereof being depicted in Table I, application no. 3, column 8, in the same line as SEQ ID NO. 75873, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 75873 is depicted, plastidic is mentioned, or a mitochondrial transit peptide in case in Table I, application no. 3, in column 6 in the same line as SEQ ID NO. 75873 is depicted, mitochondrial is mentioned; which is operable linked, too.

In another embodiment thereof the expression cassette does not comprise an additional transit peptide in case in Table I, application no. 3, in column 6 in the same line as SEQ ID NO. 75873 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7917, or a homolog or fragment thereof, or a homolog thereof being depicted in Table I, application no. 3, column 8, in the same line as SEQ ID NO. 7917, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 7917 is depicted, plastidic is mentioned, or a mitochondrial transit peptide in case in Table I, application no. 3, in column 6 in the same line as SEQ ID NO. 7917 is depicted, mitochondrial is mentioned; which is operable linked, too.

In another embodiment thereof the expression cassette does not comprise an additional transit peptide in case in Table I, application no. 3, in column 6 in the same line as SEQ ID NO. 7917 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 75915, or a homolog or fragment thereof, or a homolog thereof being depicted in Table I, application no. 3, column 8, in the same line as SEQ ID NO. 75915, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 75915 is depicted, plastidic is mentioned, or a mitochondrial transit peptide in case in Table I, application no. 3, in column 6 in the same line as SEQ ID NO. 75915 is depicted, mitochondrial is mentioned; which is operable linked, too.

In another embodiment thereof the expression cassette does not comprise an additional transit peptide in case in Table I, application no. 3, in column 6 in the same line as SEQ ID NO. 75915 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 36670, or a homolog or fragment thereof, or a homolog thereof being depicted in Table I, application no. 3, column 8, in the same line as SEQ ID NO. 36670, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 36670 is depicted, plastidic is mentioned, or a mitochondrial transit peptide in case in Table I, application no. 3, in column 6 in the same line as SEQ ID NO. 36670 is depicted, mitochondrial is mentioned; which is operable linked, too.

In another embodiment thereof the expression cassette does not comprise an additional transit peptide in case in Table I, application no. 3, in column 6 in the same line as SEQ ID NO. 36670 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 36809, or a homolog or fragment thereof, or a homolog thereof being depicted in Table I, application no. 3, column 8, in the same line as SEQ ID NO. 36809, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 36809 is depicted, plastidic is mentioned, or a mitochondrial transit peptide in case in Table I, application no. 3, in column 6 in the same line as SEQ ID NO. 36809 is depicted, mitochondrial is mentioned; which is operable linked, too.

In another embodiment thereof the expression cassette does not comprise an additional transit peptide in case in Table I, application no. 3, in column 6 in the same line as SEQ ID NO. 36809 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7941, or a homolog or fragment thereof, or a homolog thereof being depicted in Table I, application no. 3, column 8, in the same line as SEQ ID NO. 7941, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 7941 is depicted, plastidic is mentioned, or a mitochondrial transit peptide in case in Table I, application no. 3, in column 6 in the same line as SEQ ID NO. 7941 is depicted, mitochondrial is mentioned; which is operable linked, too.

In another embodiment thereof the expression cassette does not comprise an additional transit peptide in case in Table I, application no. 3, in column 6 in the same line as SEQ ID NO. 7941 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 36880, or a homolog or fragment thereof, or a homolog thereof being depicted in Table I, application no. 3, column 8, in the same line as SEQ ID NO. 36880, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 36880 is depicted, plastidic is mentioned, or a mitochondrial transit peptide in case in Table I, application no. 3, in column 6 in the same line as SEQ ID NO. 36880 is depicted, mitochondrial is mentioned; which is operable linked, too.

In another embodiment thereof the expression cassette does not comprise an additional transit peptide in case in Table I, application no. 3, in column 6 in the same line as SEQ ID NO. 36880 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 36907, or a homolog or fragment thereof, or a homolog thereof being depicted in Table I, application no. 3, column 8, in the same line as SEQ ID NO. 36907, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 36907 is depicted, plastidic is mentioned, or a mitochondrial transit peptide in case in Table I, application no. 3, in column 6 in the same line as SEQ ID NO. 36907 is depicted, mitochondrial is mentioned; which is operable linked, too.

In another embodiment thereof the expression cassette does not comprise an additional transit peptide in case in Table I, application no. 3, in column 6 in the same line as SEQ ID NO. 36907 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 76032, or a homolog or fragment thereof, or a homolog thereof being depicted in Table I, application no. 3, column 8, in the same line as SEQ ID NO. 76032, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 76032 is depicted, plastidic is mentioned, or a mitochondrial transit peptide in case in Table I, application no. 3, in column 6 in the same line as SEQ ID NO. 76032 is depicted, mitochondrial is mentioned; which is operable linked, too.

In another embodiment thereof the expression cassette does not comprise an additional transit peptide in case in Table I, application no. 3, in column 6 in the same line as SEQ ID NO. 76032 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 37390, or a homolog or fragment thereof, or a homolog thereof being depicted in Table I, application no. 3, column 8, in the same line as SEQ ID NO. 37390, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 37390 is depicted, plastidic is mentioned, or a mitochondrial transit peptide in case in Table I, application no. 3, in column 6 in the same line as SEQ ID NO. 37390 is depicted, mitochondrial is mentioned; which is operable linked, too.

In another embodiment thereof the expression cassette does not comprise an additional transit peptide in case in Table I, application no. 3, in column 6 in the same line as SEQ ID NO. 37390 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 76068, or a homolog or fragment thereof, or a homolog thereof being depicted in Table I, application no. 3, column 8, in the same line as SEQ ID NO. 76068, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 76068 is depicted, plastidic is mentioned, or a mitochondrial transit peptide in case in Table I, application no. 3, in column 6 in the same line as SEQ ID NO. 76068 is depicted, mitochondrial is mentioned; which is operable linked, too.

In another embodiment thereof the expression cassette does not comprise an additional transit peptide in case in Table I, application no. 3, in column 6 in the same line as SEQ ID NO. 76068 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 37394, or a homolog or fragment thereof, or a homolog thereof being depicted in Table I, application no. 3, column 8, in the same line as SEQ ID NO. 37394, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 37394 is depicted, plastidic is mentioned, or a mitochondrial transit peptide in case in Table I, application no. 3, in column 6 in the same line as SEQ ID NO. 37394 is depicted, mitochondrial is mentioned; which is operable linked, too.

In another embodiment thereof the expression cassette does not comprise an additional transit peptide in case in Table I, application no. 3, in column 6 in the same line as SEQ ID NO. 37394 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 76087, or a homolog or fragment thereof, or a homolog thereof being depicted in Table I, application no. 3, column 8, in the same line as SEQ ID NO. 76087, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 76087 is depicted, plastidic is mentioned, or a mitochondrial transit peptide in case in Table I, application no. 3, in column 6 in the same line as SEQ ID NO. 76087 is depicted, mitochondrial is mentioned; which is operable linked, too.

In another embodiment thereof the expression cassette does not comprise an additional transit peptide in case in Table I, application no. 3, in column 6 in the same line as SEQ ID NO. 76087 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 37400, or a homolog or fragment thereof, or a homolog thereof being depicted in Table I, application no. 3, column 8, in the same line as SEQ ID NO. 37400, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 37400 is depicted, plastidic is mentioned, or a mitochondrial transit peptide in case in Table I, application no. 3, in column 6 in the same line as SEQ ID NO. 37400 is depicted, mitochondrial is mentioned; which is operable linked, too.

In another embodiment thereof the expression cassette does not comprise an additional transit peptide in case in Table I, application no. 3, in column 6 in the same line as SEQ ID NO. 37400 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 76138, or a homolog or fragment thereof, or a homolog thereof being depicted in Table I, application no. 3, column 8, in the same line as SEQ ID NO. 76138, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 76138 is depicted, plastidic is mentioned, or a mitochondrial transit peptide in case in Table I, application no. 3, in column 6 in the same line as SEQ ID NO. 76138 is depicted, mitochondrial is mentioned; which is operable linked, too.

In another embodiment thereof the expression cassette does not comprise an additional transit peptide in case in Table I, application no. 3, in column 6 in the same line as SEQ ID NO. 76138 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 76151, or a homolog or fragment thereof, or a homolog thereof being depicted in Table I, application no. 3, column 8, in the same line as SEQ ID NO. 76151, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 76151 is depicted, plastidic is mentioned, or a mitochondrial transit peptide in case in Table I, application no. 3, in column 6 in the same line as SEQ ID NO. 76151 is depicted, mitochondrial is mentioned; which is operable linked, too.

In another embodiment thereof the expression cassette does not comprise an additional transit peptide in case in Table I, application no. 3, in column 6 in the same line as SEQ ID NO. 76151 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 37483, or a homolog or fragment thereof, or a homolog thereof being depicted in Table I, application no. 3, column 8, in the same line as SEQ ID NO. 37483, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 37483 is depicted, plastidic is mentioned, or a mitochondrial transit peptide in case in Table I, application no. 3, in column 6 in the same line as SEQ ID NO. 37483 is depicted, mitochondrial is mentioned; which is operable linked, too.

In another embodiment thereof the expression cassette does not comprise an additional transit peptide in case in Table I, application no. 3, in column 6 in the same line as SEQ ID NO. 37483 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 37503, or a homolog or fragment thereof, or a homolog thereof being depicted in Table I, application no. 3, column 8, in the same line as SEQ ID NO. 37503, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 37503 is depicted, plastidic is mentioned, or a mitochondrial transit peptide in case in Table I, application no. 3, in column 6 in the same line as SEQ ID NO. 37503 is depicted, mitochondrial is mentioned; which is operable linked, too.

In another embodiment thereof the expression cassette does not comprise an additional transit peptide in case in Table I, application no. 3, in column 6 in the same line as SEQ ID NO. 37503 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 76157, or a homolog or fragment thereof, or a homolog thereof being depicted in Table I, application no. 3, column 8, in the same line as SEQ ID NO. 76157, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 76157 is depicted, plastidic is mentioned, or a mitochondrial transit peptide in case in Table I, application no. 3, in column 6 in the same line as SEQ ID NO. 76157 is depicted, mitochondrial is mentioned; which is operable linked, too.

In another embodiment thereof the expression cassette does not comprise an additional transit peptide in case in Table I, application no. 3, in column 6 in the same line as SEQ ID NO. 76157 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 76240, or a homolog or fragment thereof, or a homolog thereof being depicted in Table I, application no. 3, column 8, in the same line as SEQ ID NO. 76240, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 76240 is depicted, plastidic is mentioned, or a mitochondrial transit peptide in case in Table I, application no. 3, in column 6 in the same line as SEQ ID NO. 76240 is depicted, mitochondrial is mentioned; which is operable linked, too.

In another embodiment thereof the expression cassette does not comprise an additional transit peptide in case in Table I, application no. 3, in column 6 in the same line as SEQ ID NO. 76240 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 37573, or a homolog or fragment thereof, or a homolog thereof being depicted in Table I, application no. 3, column 8, in the same line as SEQ ID NO. 37573, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 37573 is depicted, plastidic is mentioned, or a mitochondrial transit peptide in case in Table I, application no. 3, in column 6 in the same line as SEQ ID NO. 37573 is depicted, mitochondrial is mentioned; which is operable linked, too.

In another embodiment thereof the expression cassette does not comprise an additional transit peptide in case in Table I, application no. 3, in column 6 in the same line as SEQ ID NO. 37573 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 37658, or a homolog or fragment thereof, or a homolog thereof being depicted in Table I, application no. 3, column 8, in the same line as SEQ ID NO. 37658, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 37658 is depicted, plastidic is mentioned, or a mitochondrial transit peptide in case in Table I, application no. 3, in column 6 in the same line as SEQ ID NO. 37658 is depicted, mitochondrial is mentioned; which is operable linked, too.

In another embodiment thereof the expression cassette does not comprise an additional transit peptide in case in Table I, application no. 3, in column 6 in the same line as SEQ ID NO. 37658 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 37807, or a homolog or fragment thereof, or a homolog thereof being depicted in Table I, application no. 3, column 8, in the same line as SEQ ID NO. 37807, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 37807 is depicted, plastidic is mentioned, or a mitochondrial transit peptide in case in Table I, application no. 3, in column 6 in the same line as SEQ ID NO. 37807 is depicted, mitochondrial is mentioned; which is operable linked, too.

In another embodiment thereof the expression cassette does not comprise an additional transit peptide in case in Table I, application no. 3, in column 6 in the same line as SEQ ID NO. 37807 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 76867, or a homolog or fragment thereof, or a homolog thereof being depicted in Table I, application no. 3, column 8, in the same line as SEQ ID NO. 76867, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 76867 is depicted, plastidic is mentioned, or a mitochondrial transit peptide in case in Table I, application no. 3, in column 6 in the same line as SEQ ID NO. 76867 is depicted, mitochondrial is mentioned; which is operable linked, too.

In another embodiment thereof the expression cassette does not comprise an additional transit peptide in case in Table I, application no. 3, in column 6 in the same line as SEQ ID NO. 76867 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38226, or a homolog or fragment thereof, or a homolog thereof being depicted in Table I, application no. 3, column 8, in the same line as SEQ ID NO. 38226, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 38226 is depicted, plastidic is mentioned, or a mitochondrial transit peptide in case in Table I, application no. 3, in column 6 in the same line as SEQ ID NO. 38226 is depicted, mitochondrial is mentioned; which is operable linked, too.

In another embodiment thereof the expression cassette does not comprise an additional transit peptide in case in Table I, application no. 3, in column 6 in the same line as SEQ ID NO. 38226 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 76890, or a homolog or fragment thereof, or a homolog thereof being depicted in Table I, application no. 3, column 8, in the same line as SEQ ID NO. 76890, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 76890 is depicted, plastidic is mentioned, or a mitochondrial transit peptide in case in Table I, application no. 3, in column 6 in the same line as SEQ ID NO. 76890 is depicted, mitochondrial is mentioned; which is operable linked, too.

In another embodiment thereof the expression cassette does not comprise an additional transit peptide in case in Table I, application no. 3, in column 6 in the same line as SEQ ID NO. 76890 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 77768, or a homolog or fragment thereof, or a homolog thereof being depicted in Table I, application no. 3, column 8, in the same line as SEQ ID NO. 77768, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 77768 is depicted, plastidic is mentioned, or a mitochondrial transit peptide in case in Table I, application no. 3, in column 6 in the same line as SEQ ID NO. 77768 is depicted, mitochondrial is mentioned; which is operable linked, too.

In another embodiment thereof the expression cassette does not comprise an additional transit peptide in case in Table I, application no. 3, in column 6 in the same line as SEQ ID NO. 77768 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38266, or a homolog or fragment thereof, or a homolog thereof being depicted in Table I, application no. 3, column 8, in the same line as SEQ ID NO. 38266, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 38266 is depicted, plastidic is mentioned, or a mitochondrial transit peptide in case in Table I, application no. 3, in column 6 in the same line as SEQ ID NO. 38266 is depicted, mitochondrial is mentioned; which is operable linked, too.

In another embodiment thereof the expression cassette does not comprise an additional transit peptide in case in Table I, application no. 3, in column 6 in the same line as SEQ ID NO. 38266 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38289, or a homolog or fragment thereof, or a homolog thereof being depicted in Table I, application no. 3, column 8, in the same line as SEQ ID NO. 38289, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 38289 is depicted, plastidic is mentioned, or a mitochondrial transit peptide in case in Table I, application no. 3, in column 6 in the same line as SEQ ID NO. 38289 is depicted, mitochondrial is mentioned; which is operable linked, too.

In another embodiment thereof the expression cassette does not comprise an additional transit peptide in case in Table I, application no. 3, in column 6 in the same line as SEQ ID NO. 38289 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 77774, or a homolog or fragment thereof, or a homolog thereof being depicted in Table I, application no. 3, column 8, in the same line as SEQ ID NO. 77774, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 77774 is depicted, plastidic is mentioned, or a mitochondrial transit peptide in case in Table I, application no. 3, in column 6 in the same line as SEQ ID NO. 77774 is depicted, mitochondrial is mentioned; which is operable linked, too.

In another embodiment thereof the expression cassette does not comprise an additional transit peptide in case in Table I, application no. 3, in column 6 in the same line as SEQ ID NO. 77774 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 77774, or a homolog or fragment thereof, or a homolog thereof being depicted in Table I, application no. 3, column 8, in the same line as SEQ ID NO. 77774, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 77774 is depicted, plastidic is mentioned, or a mitochondrial transit peptide in case in Table I, application no. 3, in column 6 in the same line as SEQ ID NO. 77774 is depicted, mitochondrial is mentioned; which is operable linked, too.

In another embodiment thereof the expression cassette does not comprise an additional transit peptide in case in Table I, application no. 3, in column 6 in the same line as SEQ ID NO. 77774 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7947, or a homolog or fragment thereof, or a homolog thereof being depicted in Table I, application no. 3, column 8, in the same line as SEQ ID NO. 7947, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 7947 is depicted, plastidic is mentioned, or a mitochondrial transit peptide in case in Table I, application no. 3, in column 6 in the same line as SEQ ID NO. 7947 is depicted, mitochondrial is mentioned; which is operable linked, too.

In another embodiment thereof the expression cassette does not comprise an additional transit peptide in case in Table I, application no. 3, in column 6 in the same line as SEQ ID NO. 7947 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78138, or a homolog or fragment thereof, or a homolog thereof being depicted in Table I, application no. 3, column 8, in the same line as SEQ ID NO. 78138, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 78138 is depicted, plastidic is mentioned, or a mitochondrial transit peptide in case in Table I, application no. 3, in column 6 in the same line as SEQ ID NO. 78138 is depicted, mitochondrial is mentioned; which is operable linked, too.

In another embodiment thereof the expression cassette does not comprise an additional transit peptide in case in Table I, application no. 3, in column 6 in the same line as SEQ ID NO. 78138 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78154, or a homolog or fragment thereof, or a homolog thereof being depicted in Table I, application no. 3, column 8, in the same line as SEQ ID NO. 78154, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 78154 is depicted, plastidic is mentioned, or a mitochondrial transit peptide in case in Table I, application no. 3, in column 6 in the same line as SEQ ID NO. 78154 is depicted, mitochondrial is mentioned; which is operable linked, too.

In another embodiment thereof the expression cassette does not comprise an additional transit peptide in case in Table I, application no. 3, in column 6 in the same line as SEQ ID NO. 78154 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38300, or a homolog or fragment thereof, or a homolog thereof being depicted in Table I, application no. 3, column 8, in the same line as SEQ ID NO. 38300, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 38300 is depicted, plastidic is mentioned, or a mitochondrial transit peptide in case in Table I, application no. 3, in column 6 in the same line as SEQ ID NO. 38300 is depicted, mitochondrial is mentioned; which is operable linked, too.

In another embodiment thereof the expression cassette does not comprise an additional transit peptide in case in Table I, application no. 3, in column 6 in the same line as SEQ ID NO. 38300 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78265, or a homolog or fragment thereof, or a homolog thereof being depicted in Table I, application no. 3, column 8, in the same line as SEQ ID NO. 78265, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 78265 is depicted, plastidic is mentioned, or a mitochondrial transit peptide in case in Table I, application no. 3, in column 6 in the same line as SEQ ID NO. 78265 is depicted, mitochondrial is mentioned; which is operable linked, too.

In another embodiment thereof the expression cassette does not comprise an additional transit peptide in case in Table I, application no. 3, in column 6 in the same line as SEQ ID NO. 78265 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78753, or a homolog or fragment thereof, or a homolog thereof being depicted in Table I, application no. 3, column 8, in the same line as SEQ ID NO. 78753, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 78753 is depicted, plastidic is mentioned, or a mitochondrial transit peptide in case in Table I, application no. 3, in column 6 in the same line as SEQ ID NO. 78753 is depicted, mitochondrial is mentioned; which is operable linked, too.

In another embodiment thereof the expression cassette does not comprise an additional transit peptide in case in Table I, application no. 3, in column 6 in the same line as SEQ ID NO. 78753 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78771, or a homolog or fragment thereof, or a homolog thereof being depicted in Table I, application no. 3, column 8, in the same line as SEQ ID NO. 78771, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 78771 is depicted, plastidic is mentioned, or a mitochondrial transit peptide in case in Table I, application no. 3, in column 6 in the same line as SEQ ID NO. 78771 is depicted, mitochondrial is mentioned; which is operable linked, too.

In another embodiment thereof the expression cassette does not comprise an additional transit peptide in case in Table I, application no. 3, in column 6 in the same line as SEQ ID NO. 78771 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 78779, or a homolog or fragment thereof, or a homolog thereof being depicted in Table I, application no. 3, column 8, in the same line as SEQ ID NO. 78779, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 78779 is depicted, plastidic is mentioned, or a mitochondrial transit peptide in case in Table I, application no. 3, in column 6 in the same line as SEQ ID NO. 78779 is depicted, mitochondrial is mentioned; which is operable linked, too.

In another embodiment thereof the expression cassette does not comprise an additional transit peptide in case in Table I, application no. 3, in column 6 in the same line as SEQ ID NO. 78779 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78852, or a homolog or fragment thereof, or a homolog thereof being depicted in Table I, application no. 3, column 8, in the same line as SEQ ID NO. 78852, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 78852 is depicted, plastidic is mentioned, or a mitochondrial transit peptide in case in Table I, application no. 3, in column 6 in the same line as SEQ ID NO. 78852 is depicted, mitochondrial is mentioned; which is operable linked, too.

In another embodiment thereof the expression cassette does not comprise an additional transit peptide in case in Table I, application no. 3, in column 6 in the same line as SEQ ID NO. 78852 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78883, or a homolog or fragment thereof, or a homolog thereof being depicted in Table I, application no. 3, column 8, in the same line as SEQ ID NO. 78883, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 78883 is depicted, plastidic is mentioned, or a mitochondrial transit peptide in case in Table I, application no. 3, in column 6 in the same line as SEQ ID NO. 78883 is depicted, mitochondrial is mentioned; which is operable linked, too.

In another embodiment thereof the expression cassette does not comprise an additional transit peptide in case in Table I, application no. 3, in column 6 in the same line as SEQ ID NO. 78883 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78953, or a homolog or fragment thereof, or a homolog thereof being depicted in Table I, application no. 3, column 8, in the same line as SEQ ID NO. 78953, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 78953 is depicted, plastidic is mentioned, or a mitochondrial transit peptide in case in Table I, application no. 3, in column 6 in the same line as SEQ ID NO. 78953 is depicted, mitochondrial is mentioned; which is operable linked, too.

In another embodiment thereof the expression cassette does not comprise an additional transit peptide in case in Table I, application no. 3, in column 6 in the same line as SEQ ID NO. 78953 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 8316, or a homolog or fragment thereof, or a homolog thereof being depicted in Table I, application no. 3, column 8, in the same line as SEQ ID NO. 8316, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 8316 is depicted, plastidic is mentioned, or a mitochondrial transit peptide in case in Table I, application no. 3, in column 6 in the same line as SEQ ID NO. 8316 is depicted, mitochondrial is mentioned; which is operable linked, too.

In another embodiment thereof the expression cassette does not comprise an additional transit peptide in case in Table I, application no. 3, in column 6 in the same line as SEQ ID NO. 8316 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38573, or a homolog or fragment thereof, or a homolog thereof being depicted in Table I, application no. 3, column 8, in the same line as SEQ ID NO. 38573, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 38573 is depicted, plastidic is mentioned, or a mitochondrial transit peptide in case in Table I, application no. 3, in column 6 in the same line as SEQ ID NO. 38573 is depicted, mitochondrial is mentioned; which is operable linked, too.

In another embodiment thereof the expression cassette does not comprise an additional transit peptide in case in Table I, application no. 3, in column 6 in the same line as SEQ ID NO. 38573 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 8363, or a homolog or fragment thereof, or a homolog thereof being depicted in Table I, application no. 3, column 8, in the same line as SEQ ID NO. 8363, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 8363 is depicted, plastidic is mentioned, or a mitochondrial transit peptide in case in Table I, application no. 3, in column 6 in the same line as SEQ ID NO. 8363 is depicted, mitochondrial is mentioned; which is operable linked, too.

In another embodiment thereof the expression cassette does not comprise an additional transit peptide in case in Table I, application no. 3, in column 6 in the same line as SEQ ID NO. 8363 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78970, or a homolog or fragment thereof, or a homolog thereof being depicted in Table I, application no. 3, column 8, in the same line as SEQ ID NO. 78970, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 78970 is depicted, plastidic is mentioned, or a mitochondrial transit peptide in case in Table I, application no. 3, in column 6 in the same line as SEQ ID NO. 78970 is depicted, mitochondrial is mentioned; which is operable linked, too.

In another embodiment thereof the expression cassette does not comprise an additional transit peptide in case in Table I, application no. 3, in column 6 in the same line as SEQ ID NO. 78970 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 79056, or a homolog or fragment thereof, or a homolog thereof being depicted in Table I, application no. 3, column 8, in the same line as SEQ ID NO. 79056, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 79056 is depicted, plastidic is mentioned, or a mitochondrial transit peptide in case in Table I, application no. 3, in column 6 in the same line as SEQ ID NO. 79056 is depicted, mitochondrial is mentioned; which is operable linked, too.

In another embodiment thereof the expression cassette does not comprise an additional transit peptide in case in Table I, application no. 3, in column 6 in the same line as SEQ ID NO. 79056 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38767, or a homolog or fragment thereof, or a homolog thereof being depicted in Table I, application no. 3, column 8, in the same line as SEQ ID NO. 38767, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 38767 is depicted, plastidic is mentioned, or a mitochondrial transit peptide in case in Table I, application no. 3, in column 6 in the same line as SEQ ID NO. 38767 is depicted, mitochondrial is mentioned; which is operable linked, too.

In another embodiment thereof the expression cassette does not comprise an additional transit peptide in case in Table I, application no. 3, in column 6 in the same line as SEQ ID NO. 38767 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 79181, or a homolog or fragment thereof, or a homolog thereof being depicted in Table I, application no. 3, column 8, in the same line as SEQ ID NO. 79181, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 79181 is depicted, plastidic is mentioned, or a mitochondrial transit peptide in case in Table I, application no. 3, in column 6 in the same line as SEQ ID NO. 79181 is depicted, mitochondrial is mentioned; which is operable linked, too.

In another embodiment thereof the expression cassette does not comprise an additional transit peptide in case in Table I, application no. 3, in column 6 in the same line as SEQ ID NO. 79181 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38899, or a homolog or fragment thereof, or a homolog thereof being depicted in Table I, application no. 3, column 8, in the same line as SEQ ID NO. 38899, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 38899 is depicted, plastidic is mentioned, or a mitochondrial transit peptide in case in Table I, application no. 3, in column 6 in the same line as SEQ ID NO. 38899 is depicted, mitochondrial is mentioned; which is operable linked, too.

In another embodiment thereof the expression cassette does not comprise an additional transit peptide in case in Table I, application no. 3, in column 6 in the same line as SEQ ID NO. 38899 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 79217, or a homolog or fragment thereof, or a homolog thereof being depicted in Table I, application no. 3, column 8, in the same line as SEQ ID NO. 79217, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 79217 is depicted, plastidic is mentioned, or a mitochondrial transit peptide in case in Table I, application no. 3, in column 6 in the same line as SEQ ID NO. 79217 is depicted, mitochondrial is mentioned; which is operable linked, too.

In another embodiment thereof the expression cassette does not comprise an additional transit peptide in case in Table I, application no. 3, in column 6 in the same line as SEQ ID NO. 79217 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 8920, or a homolog or fragment thereof, or a homolog thereof being depicted in Table I, application no. 3, column 8, in the same line as SEQ ID NO. 8920, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 8920 is depicted, plastidic is mentioned, or a mitochondrial transit peptide in case in Table I, application no. 3, in column 6 in the same line as SEQ ID NO. 8920 is depicted, mitochondrial is mentioned; which is operable linked, too.

In another embodiment thereof the expression cassette does not comprise an additional transit peptide in case in Table I, application no. 3, in column 6 in the same line as SEQ ID NO. 8920 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38947, or a homolog or fragment thereof, or a homolog thereof being depicted in Table I, application no. 3, column 8, in the same line as SEQ ID NO. 38947, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 38947 is depicted, plastidic is mentioned, or a mitochondrial transit peptide in case in Table I, application no. 3, in column 6 in the same line as SEQ ID NO. 38947 is depicted, mitochondrial is mentioned; which is operable linked, too.

In another embodiment thereof the expression cassette does not comprise an additional transit peptide in case in Table I, application no. 3, in column 6 in the same line as SEQ ID NO. 38947 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 8937, or a homolog or fragment thereof, or a homolog thereof being depicted in Table I, application no. 3, column 8, in the same line as SEQ ID NO. 8937, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 8937 is depicted, plastidic is mentioned, or a mitochondrial transit peptide in case in Table I, application no. 3, in column 6 in the same line as SEQ ID NO. 8937 is depicted, mitochondrial is mentioned; which is operable linked, too.

In another embodiment thereof the expression cassette does not comprise an additional transit peptide in case in Table I, application no. 3, in column 6 in the same line as SEQ ID NO. 8937 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39002, or a homolog or fragment thereof, or a homolog thereof being depicted in Table I, application no. 3, column 8, in the same line as SEQ ID NO. 39002, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 39002 is depicted, plastidic is mentioned, or a mitochondrial transit peptide in case in Table I, application no. 3, in column 6 in the same line as SEQ ID NO. 39002 is depicted, mitochondrial is mentioned; which is operable linked, too.

In another embodiment thereof the expression cassette does not comprise an additional transit peptide in case in Table I, application no. 3, in column 6 in the same line as SEQ ID NO. 39002 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39040, or a homolog or fragment thereof, or a homolog thereof being depicted in Table I, application no. 3, column 8, in the same line as SEQ ID NO. 39040, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 39040 is depicted, plastidic is mentioned, or a mitochondrial transit peptide in case in Table I, application no. 3, in column 6 in the same line as SEQ ID NO. 39040 is depicted, mitochondrial is mentioned; which is operable linked, too.

In another embodiment thereof the expression cassette does not comprise an additional transit peptide in case in Table I, application no. 3, in column 6 in the same line as SEQ ID NO. 39040 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 79279, or a homolog or fragment thereof, or a homolog thereof being depicted in Table I, application no. 3, column 8, in the same line as SEQ ID NO. 79279, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 79279 is depicted, plastidic is mentioned, or a mitochondrial transit peptide in case in Table I, application no. 3, in column 6 in the same line as SEQ ID NO. 79279 is depicted, mitochondrial is mentioned; which is operable linked, too.

In another embodiment thereof the expression cassette does not comprise an additional transit peptide in case in Table I, application no. 3, in column 6 in the same line as SEQ ID NO. 79279 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 39120, or a homolog or fragment thereof, or a homolog thereof being depicted in Table I, application no. 3, column 8, in the same line as SEQ ID NO. 39120, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 39120 is depicted, plastidic is mentioned, or a mitochondrial transit peptide in case in Table I, application no. 3, in column 6 in the same line as SEQ ID NO. 39120 is depicted, mitochondrial is mentioned; which is operable linked, too.

In another embodiment thereof the expression cassette does not comprise an additional transit peptide in case in Table I, application no. 3, in column 6 in the same line as SEQ ID NO. 39120 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39219, or a homolog or fragment thereof, or a homolog thereof being depicted in Table I, application no. 3, column 8, in the same line as SEQ ID NO. 39219, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 39219 is depicted, plastidic is mentioned, or a mitochondrial transit peptide in case in Table I, application no. 3, in column 6 in the same line as SEQ ID NO. 39219 is depicted, mitochondrial is mentioned; which is operable linked, too.

In another embodiment thereof the expression cassette does not comprise an additional transit peptide in case in Table I, application no. 3, in column 6 in the same line as SEQ ID NO. 39219 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 79342, or a homolog or fragment thereof, or a homolog thereof being depicted in Table I, application no. 3, column 8, in the same line as SEQ ID NO. 79342, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 79342 is depicted, plastidic is mentioned, or a mitochondrial transit peptide in case in Table I, application no. 3, in column 6 in the same line as SEQ ID NO. 79342 is depicted, mitochondrial is mentioned; which is operable linked, too.

In another embodiment thereof the expression cassette does not comprise an additional transit peptide in case in Table I, application no. 3, in column 6 in the same line as SEQ ID NO. 79342 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39237, or a homolog or fragment thereof, or a homolog thereof being depicted in Table I, application no. 3, column 8, in the same line as SEQ ID NO. 39237, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 39237 is depicted, plastidic is mentioned, or a mitochondrial transit peptide in case in Table I, application no. 3, in column 6 in the same line as SEQ ID NO. 39237 is depicted, mitochondrial is mentioned; which is operable linked, too.

In another embodiment thereof the expression cassette does not comprise an additional transit peptide in case in Table I, application no. 3, in column 6 in the same line as SEQ ID NO. 39237 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39255, or a homolog or fragment thereof, or a homolog thereof being depicted in Table I, application no. 3, column 8, in the same line as SEQ ID NO. 39255, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 39255 is depicted, plastidic is mentioned, or a mitochondrial transit peptide in case in Table I, application no. 3, in column 6 in the same line as SEQ ID NO. 39255 is depicted, mitochondrial is mentioned; which is operable linked, too.

In another embodiment thereof the expression cassette does not comprise an additional transit peptide in case in Table I, application no. 3, in column 6 in the same line as SEQ ID NO. 39255 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 79398, or a homolog or fragment thereof, or a homolog thereof being depicted in Table I, application no. 3, column 8, in the same line as SEQ ID NO. 79398, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 79398 is depicted, plastidic is mentioned, or a mitochondrial transit peptide in case in Table I, application no. 3, in column 6 in the same line as SEQ ID NO. 79398 is depicted, mitochondrial is mentioned; which is operable linked, too.

In another embodiment thereof the expression cassette does not comprise an additional transit peptide in case in Table I, application no. 3, in column 6 in the same line as SEQ ID NO. 79398 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40329, or a homolog or fragment thereof, or a homolog thereof being depicted in Table I, application no. 3, column 8, in the same line as SEQ ID NO. 40329, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 40329 is depicted, plastidic is mentioned, or a mitochondrial transit peptide in case in Table I, application no. 3, in column 6 in the same line as SEQ ID NO. 40329 is depicted, mitochondrial is mentioned; which is operable linked, too.

In another embodiment thereof the expression cassette does not comprise an additional transit peptide in case in Table I, application no. 3, in column 6 in the same line as SEQ ID NO. 40329 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 79509, or a homolog or fragment thereof, or a homolog thereof being depicted in Table I, application no. 3, column 8, in the same line as SEQ ID NO. 79509, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 79509 is depicted, plastidic is mentioned, or a mitochondrial transit peptide in case in Table I, application no. 3, in column 6 in the same line as SEQ ID NO. 79509 is depicted, mitochondrial is mentioned; which is operable linked, too.

In another embodiment thereof the expression cassette does not comprise an additional transit peptide in case in Table I, application no. 3, in column 6 in the same line as SEQ ID NO. 79509 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 9167, or a homolog or fragment thereof, or a homolog thereof being depicted in Table I, application no. 3, column 8, in the same line as SEQ ID NO. 9167, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 9167 is depicted, plastidic is mentioned, or a mitochondrial transit peptide in case in Table I, application no. 3, in column 6 in the same line as SEQ ID NO. 9167 is depicted, mitochondrial is mentioned; which is operable linked, too.

In another embodiment thereof the expression cassette does not comprise an additional transit peptide in case in Table I, application no. 3, in column 6 in the same line as SEQ ID NO. 9167 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 79627, or a homolog or fragment thereof, or a homolog thereof being depicted in Table I, application no. 3, column 8, in the same line as SEQ ID NO. 79627, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 79627 is depicted, plastidic is mentioned, or a mitochondrial transit peptide in case in Table I, application no. 3, in column 6 in the same line as SEQ ID NO. 79627 is depicted, mitochondrial is mentioned; which is operable linked, too.

In another embodiment thereof the expression cassette does not comprise an additional transit peptide in case in Table I, application no. 3, in column 6 in the same line as SEQ ID NO. 79627 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 80521, or a homolog or fragment thereof, or a homolog thereof being depicted in Table I, application no. 3, column 8, in the same line as SEQ ID NO. 80521, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 80521 is depicted, plastidic is mentioned, or a mitochondrial transit peptide in case in Table I, application no. 3, in column 6 in the same line as SEQ ID NO. 80521 is depicted, mitochondrial is mentioned; which is operable linked, too.

In another embodiment thereof the expression cassette does not comprise an additional transit peptide in case in Table I, application no. 3, in column 6 in the same line as SEQ ID NO. 80521 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40665, or a homolog or fragment thereof, or a homolog thereof being depicted in Table I, application no. 3, column 8, in the same line as SEQ ID NO. 40665, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 40665 is depicted, plastidic is mentioned, or a mitochondrial transit peptide in case in Table I, application no. 3, in column 6 in the same line as SEQ ID NO. 40665 is depicted, mitochondrial is mentioned; which is operable linked, too.

In another embodiment thereof the expression cassette does not comprise an additional transit peptide in case in Table I, application no. 3, in column 6 in the same line as SEQ ID NO. 40665 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40726, or a homolog or fragment thereof, or a homolog thereof being depicted in Table I, application no. 3, column 8, in the same line as SEQ ID NO. 40726, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 40726 is depicted, plastidic is mentioned, or a mitochondrial transit peptide in case in Table I, application no. 3, in column 6 in the same line as SEQ ID NO. 40726 is depicted, mitochondrial is mentioned; which is operable linked, too.

In another embodiment thereof the expression cassette does not comprise an additional transit peptide in case in Table I, application no. 3, in column 6 in the same line as SEQ ID NO. 40726 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 9244, or a homolog or fragment thereof, or a homolog thereof being depicted in Table I, application no. 3, column 8, in the same line as SEQ ID NO. 9244, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 9244 is depicted, plastidic is mentioned, or a mitochondrial transit peptide in case in Table I, application no. 3, in column 6 in the same line as SEQ ID NO. 9244 is depicted, mitochondrial is mentioned; which is operable linked, too.

In another embodiment thereof the expression cassette does not comprise an additional transit peptide in case in Table I, application no. 3, in column 6 in the same line as SEQ ID NO. 9244 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 80589, or a homolog or fragment thereof, or a homolog thereof being depicted in Table I, application no. 3, column 8, in the same line as SEQ ID NO. 80589, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 80589 is depicted, plastidic is mentioned, or a mitochondrial transit peptide in case in Table I, application no. 3, in column 6 in the same line as SEQ ID NO. 80589 is depicted, mitochondrial is mentioned; which is operable linked, too.

In another embodiment thereof the expression cassette does not comprise an additional transit peptide in case in Table I, application no. 3, in column 6 in the same line as SEQ ID NO. 80589 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 80756, or a homolog or fragment thereof, or a homolog thereof being depicted in Table I, application no. 3, column 8, in the same line as SEQ ID NO. 80756, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 80756 is depicted, plastidic is mentioned, or a mitochondrial transit peptide in case in Table I, application no. 3, in column 6 in the same line as SEQ ID NO. 80756 is depicted, mitochondrial is mentioned; which is operable linked, too.

In another embodiment thereof the expression cassette does not comprise an additional transit peptide in case in Table I, application no. 3, in column 6 in the same line as SEQ ID NO. 80756 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40741, or a homolog or fragment thereof, or a homolog thereof being depicted in Table I, application no. 3, column 8, in the same line as SEQ ID NO. 40741, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 40741 is depicted, plastidic is mentioned, or a mitochondrial transit peptide in case in Table I, application no. 3, in column 6 in the same line as SEQ ID NO. 40741 is depicted, mitochondrial is mentioned; which is operable linked, too.

In another embodiment thereof the expression cassette does not comprise an additional transit peptide in case in Table I, application no. 3, in column 6 in the same line as SEQ ID NO. 40741 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 80906, or a homolog or fragment thereof, or a homolog thereof being depicted in Table I, application no. 3, column 8, in the same line as SEQ ID NO. 80906, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 80906 is depicted, plastidic is mentioned, or a mitochondrial transit peptide in case in Table I, application no. 3, in column 6 in the same line as SEQ ID NO. 80906 is depicted, mitochondrial is mentioned; which is operable linked, too.

In another embodiment thereof the expression cassette does not comprise an additional transit peptide in case in Table I, application no. 3, in column 6 in the same line as SEQ ID NO. 80906 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 81075, or a homolog or fragment thereof, or a homolog thereof being depicted in Table I, application no. 3, column 8, in the same line as SEQ ID NO. 81075, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 81075 is depicted, plastidic is mentioned, or a mitochondrial transit peptide in case in Table I, application no. 3, in column 6 in the same line as SEQ ID NO. 81075 is depicted, mitochondrial is mentioned; which is operable linked, too.

In another embodiment thereof the expression cassette does not comprise an additional transit peptide in case in Table I, application no. 3, in column 6 in the same line as SEQ ID NO. 81075 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 81451, or a homolog or fragment thereof, or a homolog thereof being depicted in Table I, application no. 3, column 8, in the same line as SEQ ID NO. 81451, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 81451 is depicted, plastidic is mentioned, or a mitochondrial transit peptide in case in Table I, application no. 3, in column 6 in the same line as SEQ ID NO. 81451 is depicted, mitochondrial is mentioned; which is operable linked, too.

In another embodiment thereof the expression cassette does not comprise an additional transit peptide in case in Table I, application no. 3, in column 6 in the same line as SEQ ID NO. 81451 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40795, or a homolog or fragment thereof, or a homolog thereof being depicted in Table I, application no. 3, column 8, in the same line as SEQ ID NO. 40795, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 40795 is depicted, plastidic is mentioned, or a mitochondrial transit peptide in case in Table I, application no. 3, in column 6 in the same line as SEQ ID NO. 40795 is depicted, mitochondrial is mentioned; which is operable linked, too.

In another embodiment thereof the expression cassette does not comprise an additional transit peptide in case in Table I, application no. 3, in column 6 in the same line as SEQ ID NO. 40795 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 81576, or a homolog or fragment thereof, or a homolog thereof being depicted in Table I, application no. 3, column 8, in the same line as SEQ ID NO. 81576, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 81576 is depicted, plastidic is mentioned, or a mitochondrial transit peptide in case in Table I, application no. 3, in column 6 in the same line as SEQ ID NO. 81576 is depicted, mitochondrial is mentioned; which is operable linked, too.

In another embodiment thereof the expression cassette does not comprise an additional transit peptide in case in Table I, application no. 3, in column 6 in the same line as SEQ ID NO. 81576 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 81692, or a homolog or fragment thereof, or a homolog thereof being depicted in Table I, application no. 3, column 8, in the same line as SEQ ID NO. 81692, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 81692 is depicted, plastidic is mentioned, or a mitochondrial transit peptide in case in Table I, application no. 3, in column 6 in the same line as SEQ ID NO. 81692 is depicted, mitochondrial is mentioned; which is operable linked, too.

In another embodiment thereof the expression cassette does not comprise an additional transit peptide in case in Table I, application no. 3, in column 6 in the same line as SEQ ID NO. 81692 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 81935, or a homolog or fragment thereof, or a homolog thereof being depicted in Table I, application no. 3, column 8, in the same line as SEQ ID NO. 81935, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 81935 is depicted, plastidic is mentioned, or a mitochondrial transit peptide in case in Table I, application no. 3, in column 6 in the same line as SEQ ID NO. 81935 is depicted, mitochondrial is mentioned; which is operable linked, too.

In another embodiment thereof the expression cassette does not comprise an additional transit peptide in case in Table I, application no. 3, in column 6 in the same line as SEQ ID NO. 81935 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 81980, or a homolog or fragment thereof, or a homolog thereof being depicted in Table I, application no. 3, column 8, in the same line as SEQ ID NO. 81980, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 81980 is depicted, plastidic is mentioned, or a mitochondrial transit peptide in case in Table I, application no. 3, in column 6 in the same line as SEQ ID NO. 81980 is depicted, mitochondrial is mentioned; which is operable linked, too.

In another embodiment thereof the expression cassette does not comprise an additional transit peptide in case in Table I, application no. 3, in column 6 in the same line as SEQ ID NO. 81980 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 81988, or a homolog or fragment thereof, or a homolog thereof being depicted in Table I, application no. 3, column 8, in the same line as SEQ ID NO. 81988, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 81988 is depicted, plastidic is mentioned, or a mitochondrial transit peptide in case in Table I, application no. 3, in column 6 in the same line as SEQ ID NO. 81988 is depicted, mitochondrial is mentioned; which is operable linked, too.

In another embodiment thereof the expression cassette does not comprise an additional transit peptide in case in Table I, application no. 3, in column 6 in the same line as SEQ ID NO. 81988 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 9333, or a homolog or fragment thereof, or a homolog thereof being depicted in Table I, application no. 3, column 8, in the same line as SEQ ID NO. 9333, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 9333 is depicted, plastidic is mentioned, or a mitochondrial transit peptide in case in Table I, application no. 3, in column 6 in the same line as SEQ ID NO. 9333 is depicted, mitochondrial is mentioned; which is operable linked, too.

In another embodiment thereof the expression cassette does not comprise an additional transit peptide in case in Table I, application no. 3, in column 6 in the same line as SEQ ID NO. 9333 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 42046, or a homolog or fragment thereof, or a homolog thereof being depicted in Table I, application no. 3, column 8, in the same line as SEQ ID NO. 42046, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 42046 is depicted, plastidic is mentioned, or a mitochondrial transit peptide in case in Table I, application no. 3, in column 6 in the same line as SEQ ID NO. 42046 is depicted, mitochondrial is mentioned; which is operable linked, too.

In another embodiment thereof the expression cassette does not comprise an additional transit peptide in case in Table I, application no. 3, in column 6 in the same line as SEQ ID NO. 42046 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 82424, or a homolog or fragment thereof, or a homolog thereof being depicted in Table I, application no. 3, column 8, in the same line as SEQ ID NO. 82424, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 82424 is depicted, plastidic is mentioned, or a mitochondrial transit peptide in case in Table I, application no. 3, in column 6 in the same line as SEQ ID NO. 82424 is depicted, mitochondrial is mentioned; which is operable linked, too.

In another embodiment thereof the expression cassette does not comprise an additional transit peptide in case in Table I, application no. 3, in column 6 in the same line as SEQ ID NO. 82424 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 82459, or a homolog or fragment thereof, or a homolog thereof being depicted in Table I, application no. 3, column 8, in the same line as SEQ ID NO. 82459, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 82459 is depicted, plastidic is mentioned, or a mitochondrial transit peptide in case in Table I, application no. 3, in column 6 in the same line as SEQ ID NO. 82459 is depicted, mitochondrial is mentioned; which is operable linked, too.

In another embodiment thereof the expression cassette does not comprise an additional transit peptide in case in Table I, application no. 3, in column 6 in the same line as SEQ ID NO. 82459 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 42471, or a homolog or fragment thereof, or a homolog thereof being depicted in Table I, application no. 3, column 8, in the same line as SEQ ID NO. 42471, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 42471 is depicted, plastidic is mentioned, or a mitochondrial transit peptide in case in Table I, application no. 3, in column 6 in the same line as SEQ ID NO. 42471 is depicted, mitochondrial is mentioned; which is operable linked, too.

In another embodiment thereof the expression cassette does not comprise an additional transit peptide in case in Table I, application no. 3, in column 6 in the same line as SEQ ID NO. 42471 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 82510, or a homolog or fragment thereof, or a homolog thereof being depicted in Table I, application no. 3, column 8, in the same line as SEQ ID NO. 82510, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 82510 is depicted, plastidic is mentioned, or a mitochondrial transit peptide in case in Table I, application no. 3, in column 6 in the same line as SEQ ID NO. 82510 is depicted, mitochondrial is mentioned; which is operable linked, too.

In another embodiment thereof the expression cassette does not comprise an additional transit peptide in case in Table I, application no. 3, in column 6 in the same line as SEQ ID NO. 82510 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 9470, or a homolog or fragment thereof, or a homolog thereof being depicted in Table I, application no. 3, column 8, in the same line as SEQ ID NO. 9470, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 9470 is depicted, plastidic is mentioned, or a mitochondrial transit peptide in case in Table I, application no. 3, in column 6 in the same line as SEQ ID NO. 9470 is depicted, mitochondrial is mentioned; which is operable linked, too.

In another embodiment thereof the expression cassette does not comprise an additional transit peptide in case in Table I, application no. 3, in column 6 in the same line as SEQ ID NO. 9470 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 9492, or a homolog or fragment thereof, or a homolog thereof being depicted in Table I, application no. 3, column 8, in the same line as SEQ ID NO. 9492, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 9492 is depicted, plastidic is mentioned, or a mitochondrial transit peptide in case in Table I, application no. 3, in column 6 in the same line as SEQ ID NO. 9492 is depicted, mitochondrial is mentioned; which is operable linked, too.

In another embodiment thereof the expression cassette does not comprise an additional transit peptide in case in Table I, application no. 3, in column 6 in the same line as SEQ ID NO. 9492 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 42502, or a homolog or fragment thereof, or a homolog thereof being depicted in Table I, application no. 3, column 8, in the same line as SEQ ID NO. 42502, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 42502 is depicted, plastidic is mentioned, or a mitochondrial transit peptide in case in Table I, application no. 3, in column 6 in the same line as SEQ ID NO. 42502 is depicted, mitochondrial is mentioned; which is operable linked, too.

In another embodiment thereof the expression cassette does not comprise an additional transit peptide in case in Table I, application no. 3, in column 6 in the same line as SEQ ID NO. 42502 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 10104, or a homolog or fragment thereof, or a homolog thereof being depicted in Table I, application no. 3, column 8, in the same line as SEQ ID NO. 10104, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 10104 is depicted, plastidic is mentioned, or a mitochondrial transit peptide in case in Table I, application no. 3, in column 6 in the same line as SEQ ID NO. 10104 is depicted, mitochondrial is mentioned; which is operable linked, too.

In another embodiment thereof the expression cassette does not comprise an additional transit peptide in case in Table I, application no. 3, in column 6 in the same line as SEQ ID NO. 10104 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 82572, or a homolog or fragment thereof, or a homolog thereof being depicted in Table I, application no. 3, column 8, in the same line as SEQ ID NO. 82572, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 82572 is depicted, plastidic is mentioned, or a mitochondrial transit peptide in case in Table I, application no. 3, in column 6 in the same line as SEQ ID NO. 82572 is depicted, mitochondrial is mentioned; which is operable linked, too.

In another embodiment thereof the expression cassette does not comprise an additional transit peptide in case in Table I, application no. 3, in column 6 in the same line as SEQ ID NO. 82572 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 82846, or a homolog or fragment thereof, or a homolog thereof being depicted in Table I, application no. 3, column 8, in the same line as SEQ ID NO. 82846, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 82846 is depicted, plastidic is mentioned, or a mitochondrial transit peptide in case in Table I, application no. 3, in column 6 in the same line as SEQ ID NO. 82846 is depicted, mitochondrial is mentioned; which is operable linked, too.

In another embodiment thereof the expression cassette does not comprise an additional transit peptide in case in Table I, application no. 3, in column 6 in the same line as SEQ ID NO. 82846 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 82866, or a homolog or fragment thereof, or a homolog thereof being depicted in Table I, application no. 3, column 8, in the same line as SEQ ID NO. 82866, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 82866 is depicted, plastidic is mentioned, or a mitochondrial transit peptide in case in Table I, application no. 3, in column 6 in the same line as SEQ ID NO. 82866 is depicted, mitochondrial is mentioned; which is operable linked, too.

In another embodiment thereof the expression cassette does not comprise an additional transit peptide in case in Table I, application no. 3, in column 6 in the same line as SEQ ID NO. 82866 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 42559, or a homolog or fragment thereof, or a homolog thereof being depicted in Table I, application no. 3, column 8, in the same line as SEQ ID NO. 42559, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 42559 is depicted, plastidic is mentioned, or a mitochondrial transit peptide in case in Table I, application no. 3, in column 6 in the same line as SEQ ID NO. 42559 is depicted, mitochondrial is mentioned; which is operable linked, too.

In another embodiment thereof the expression cassette does not comprise an additional transit peptide in case in Table I, application no. 3, in column 6 in the same line as SEQ ID NO. 42559 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 42592, or a homolog or fragment thereof, or a homolog thereof being depicted in Table I, application no. 3, column 8, in the same line as SEQ ID NO. 42592, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 42592 is depicted, plastidic is mentioned, or a mitochondrial transit peptide in case in Table I, application no. 3, in column 6 in the same line as SEQ ID NO. 42592 is depicted, mitochondrial is mentioned; which is operable linked, too.

In another embodiment thereof the expression cassette does not comprise an additional transit peptide in case in Table I, application no. 3, in column 6 in the same line as SEQ ID NO. 42592 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 83153, or a homolog or fragment thereof, or a homolog thereof being depicted in Table I, application no. 3, column 8, in the same line as SEQ ID NO. 83153, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 83153 is depicted, plastidic is mentioned, or a mitochondrial transit peptide in case in Table I, application no. 3, in column 6 in the same line as SEQ ID NO. 83153 is depicted, mitochondrial is mentioned; which is operable linked, too.

In another embodiment thereof the expression cassette does not comprise an additional transit peptide in case in Table I, application no. 3, in column 6 in the same line as SEQ ID NO. 83153 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 83213, or a homolog or fragment thereof, or a homolog thereof being depicted in Table I, application no. 3, column 8, in the same line as SEQ ID NO. 83213, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 83213 is depicted, plastidic is mentioned, or a mitochondrial transit peptide in case in Table I, application no. 3, in column 6 in the same line as SEQ ID NO. 83213 is depicted, mitochondrial is mentioned; which is operable linked, too.

In another embodiment thereof the expression cassette does not comprise an additional transit peptide in case in Table I, application no. 3, in column 6 in the same line as SEQ ID NO. 83213 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 42931, or a homolog or fragment thereof, or a homolog thereof being depicted in Table I, application no. 3, column 8, in the same line as SEQ ID NO. 42931, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 42931 is depicted, plastidic is mentioned, or a mitochondrial transit peptide in case in Table I, application no. 3, in column 6 in the same line as SEQ ID NO. 42931 is depicted, mitochondrial is mentioned; which is operable linked, too.

In another embodiment thereof the expression cassette does not comprise an additional transit peptide in case in Table I, application no. 3, in column 6 in the same line as SEQ ID NO. 42931 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 83429, or a homolog or fragment thereof, or a homolog thereof being depicted in Table I, application no. 3, column 8, in the same line as SEQ ID NO. 83429, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 83429 is depicted, plastidic is mentioned, or a mitochondrial transit peptide in case in Table I, application no. 3, in column 6 in the same line as SEQ ID NO. 83429 is depicted, mitochondrial is mentioned; which is operable linked, too.

In another embodiment thereof the expression cassette does not comprise an additional transit peptide in case in Table I, application no. 3, in column 6 in the same line as SEQ ID NO. 83429 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 83629, or a homolog or fragment thereof, or a homolog thereof being depicted in Table I, application no. 3, column 8, in the same line as SEQ ID NO. 83629, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 83629 is depicted, plastidic is mentioned, or a mitochondrial transit peptide in case in Table I, application no. 3, in column 6 in the same line as SEQ ID NO. 83629 is depicted, mitochondrial is mentioned; which is operable linked, too.

In another embodiment thereof the expression cassette does not comprise an additional transit peptide in case in Table I, application no. 3, in column 6 in the same line as SEQ ID NO. 83629 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 83657, or a homolog or fragment thereof, or a homolog thereof being depicted in Table I, application no. 3, column 8, in the same line as SEQ ID NO. 83657, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 83657 is depicted, plastidic is mentioned, or a mitochondrial transit peptide in case in Table I, application no. 3, in column 6 in the same line as SEQ ID NO. 83657 is depicted, mitochondrial is mentioned; which is operable linked, too.

In another embodiment thereof the expression cassette does not comprise an additional transit peptide in case in Table I, application no. 3, in column 6 in the same line as SEQ ID NO. 83657 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 10252, or a homolog or fragment thereof, or a homolog thereof being depicted in Table I, application no. 3, column 8, in the same line as SEQ ID NO. 10252, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 10252 is depicted, plastidic is mentioned, or a mitochondrial transit peptide in case in Table I, application no. 3, in column 6 in the same line as SEQ ID NO. 10252 is depicted, mitochondrial is mentioned; which is operable linked, too.

In another embodiment thereof the expression cassette does not comprise an additional transit peptide in case in Table I, application no. 3, in column 6 in the same line as SEQ ID NO. 10252 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 83726, or a homolog or fragment thereof, or a homolog thereof being depicted in Table I, application no. 3, column 8, in the same line as SEQ ID NO. 83726, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 83726 is depicted, plastidic is mentioned, or a mitochondrial transit peptide in case in Table I, application no. 3, in column 6 in the same line as SEQ ID NO. 83726 is depicted, mitochondrial is mentioned; which is operable linked, too.

In another embodiment thereof the expression cassette does not comprise an additional transit peptide in case in Table I, application no. 3, in column 6 in the same line as SEQ ID NO. 83726 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 43800, or a homolog or fragment thereof, or a homolog thereof being depicted in Table I, application no. 3, column 8, in the same line as SEQ ID NO. 43800, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 43800 is depicted, plastidic is mentioned, or a mitochondrial transit peptide in case in Table I, application no. 3, in column 6 in the same line as SEQ ID NO. 43800 is depicted, mitochondrial is mentioned; which is operable linked, too.

In another embodiment thereof the expression cassette does not comprise an additional transit peptide in case in Table I, application no. 3, in column 6 in the same line as SEQ ID NO. 43800 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 43839, or a homolog or fragment thereof, or a homolog thereof being depicted in Table I, application no. 3, column 8, in the same line as SEQ ID NO. 43839, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 43839 is depicted, plastidic is mentioned, or a mitochondrial transit peptide in case in Table I, application no. 3, in column 6 in the same line as SEQ ID NO. 43839 is depicted, mitochondrial is mentioned; which is operable linked, too.

In another embodiment thereof the expression cassette does not comprise an additional transit peptide in case in Table I, application no. 3, in column 6 in the same line as SEQ ID NO. 43839 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 44196, or a homolog or fragment thereof, or a homolog thereof being depicted in Table I, application no. 3, column 8, in the same line as SEQ ID NO. 44196, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 44196 is depicted, plastidic is mentioned, or a mitochondrial transit peptide in case in Table I, application no. 3, in column 6 in the same line as SEQ ID NO. 44196 is depicted, mitochondrial is mentioned; which is operable linked, too.

In another embodiment thereof the expression cassette does not comprise an additional transit peptide in case in Table I, application no. 3, in column 6 in the same line as SEQ ID NO. 44196 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 83821, or a homolog or fragment thereof, or a homolog thereof being depicted in Table I, application no. 3, column 8, in the same line as SEQ ID NO. 83821, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 83821 is depicted, plastidic is mentioned, or a mitochondrial transit peptide in case in Table I, application no. 3, in column 6 in the same line as SEQ ID NO. 83821 is depicted, mitochondrial is mentioned; which is operable linked, too.

In another embodiment thereof the expression cassette does not comprise an additional transit peptide in case in Table I, application no. 3, in column 6 in the same line as SEQ ID NO. 83821 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 44372, or a homolog or fragment thereof, or a homolog thereof being depicted in Table I, application no. 3, column 8, in the same line as SEQ ID NO. 44372, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 44372 is depicted, plastidic is mentioned, or a mitochondrial transit peptide in case in Table I, application no. 3, in column 6 in the same line as SEQ ID NO. 44372 is depicted, mitochondrial is mentioned; which is operable linked, too.

In another embodiment thereof the expression cassette does not comprise an additional transit peptide in case in Table I, application no. 3, in column 6 in the same line as SEQ ID NO. 44372 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 44378, or a homolog or fragment thereof, or a homolog thereof being depicted in Table I, application no. 3, column 8, in the same line as SEQ ID NO. 44378, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 44378 is depicted, plastidic is mentioned, or a mitochondrial transit peptide in case in Table I, application no. 3, in column 6 in the same line as SEQ ID NO. 44378 is depicted, mitochondrial is mentioned; which is operable linked, too.

In another embodiment thereof the expression cassette does not comprise an additional transit peptide in case in Table I, application no. 3, in column 6 in the same line as SEQ ID NO. 44378 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 83835, or a homolog or fragment thereof, or a homolog thereof being depicted in Table I, application no. 3, column 8, in the same line as SEQ ID NO. 83835, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 83835 is depicted, plastidic is mentioned, or a mitochondrial transit peptide in case in Table I, application no. 3, in column 6 in the same line as SEQ ID NO. 83835 is depicted, mitochondrial is mentioned; which is operable linked, too.

In another embodiment thereof the expression cassette does not comprise an additional transit peptide in case in Table I, application no. 3, in column 6 in the same line as SEQ ID NO. 83835 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 10740, or a homolog or fragment thereof, or a homolog thereof being depicted in Table I, application no. 3, column 8, in the same line as SEQ ID NO. 10740, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 10740 is depicted, plastidic is mentioned, or a mitochondrial transit peptide in case in Table I, application no. 3, in column 6 in the same line as SEQ ID NO. 10740 is depicted, mitochondrial is mentioned; which is operable linked, too.

In another embodiment thereof the expression cassette does not comprise an additional transit peptide in case in Table I, application no. 3, in column 6 in the same line as SEQ ID NO. 10740 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 44609, or a homolog or fragment thereof, or a homolog thereof being depicted in Table I, application no. 3, column 8, in the same line as SEQ ID NO. 44609, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 44609 is depicted, plastidic is mentioned, or a mitochondrial transit peptide in case in Table I, application no. 3, in column 6 in the same line as SEQ ID NO. 44609 is depicted, mitochondrial is mentioned; which is operable linked, too.

In another embodiment thereof the expression cassette does not comprise an additional transit peptide in case in Table I, application no. 3, in column 6 in the same line as SEQ ID NO. 44609 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 45321, or a homolog or fragment thereof, or a homolog thereof being depicted in Table I, application no. 3, column 8, in the same line as SEQ ID NO. 45321, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 45321 is depicted, plastidic is mentioned, or a mitochondrial transit peptide in case in Table I, application no. 3, in column 6 in the same line as SEQ ID NO. 45321 is depicted, mitochondrial is mentioned; which is operable linked, too.

In another embodiment thereof the expression cassette does not comprise an additional transit peptide in case in Table I, application no. 3, in column 6 in the same line as SEQ ID NO. 45321 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 45757, or a homolog or fragment thereof, or a homolog thereof being depicted in Table I, application no. 3, column 8, in the same line as SEQ ID NO. 45757, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 45757 is depicted, plastidic is mentioned, or a mitochondrial transit peptide in case in Table I, application no. 3, in column 6 in the same line as SEQ ID NO. 45757 is depicted, mitochondrial is mentioned; which is operable linked, too.

In another embodiment thereof the expression cassette does not comprise an additional transit peptide in case in Table I, application no. 3, in column 6 in the same line as SEQ ID NO. 45757 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 84079, or a homolog or fragment thereof, or a homolog thereof being depicted in Table I, application no. 3, column 8, in the same line as SEQ ID NO. 84079, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 84079 is depicted, plastidic is mentioned, or a mitochondrial transit peptide in case in Table I, application no. 3, in column 6 in the same line as SEQ ID NO. 84079 is depicted, mitochondrial is mentioned; which is operable linked, too.

In another embodiment thereof the expression cassette does not comprise an additional transit peptide in case in Table I, application no. 3, in column 6 in the same line as SEQ ID NO. 84079 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 45795, or a homolog or fragment thereof, or a homolog thereof being depicted in Table I, application no. 3, column 8, in the same line as SEQ ID NO. 45795, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 45795 is depicted, plastidic is mentioned, or a mitochondrial transit peptide in case in Table I, application no. 3, in column 6 in the same line as SEQ ID NO. 45795 is depicted, mitochondrial is mentioned; which is operable linked, too.

In another embodiment thereof the expression cassette does not comprise an additional transit peptide in case in Table I, application no. 3, in column 6 in the same line as SEQ ID NO. 45795 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 10811, or a homolog or fragment thereof, or a homolog thereof being depicted in Table I, application no. 3, column 8, in the same line as SEQ ID NO. 10811, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 10811 is depicted, plastidic is mentioned, or a mitochondrial transit peptide in case in Table I, application no. 3, in column 6 in the same line as SEQ ID NO. 10811 is depicted, mitochondrial is mentioned; which is operable linked, too.

In another embodiment thereof the expression cassette does not comprise an additional transit peptide in case in Table I, application no. 3, in column 6 in the same line as SEQ ID NO. 10811 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 45897, or a homolog or fragment thereof, or a homolog thereof being depicted in Table I, application no. 3, column 8, in the same line as SEQ ID NO. 45897, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 45897 is depicted, plastidic is mentioned, or a mitochondrial transit peptide in case in Table I, application no. 3, in column 6 in the same line as SEQ ID NO. 45897 is depicted, mitochondrial is mentioned; which is operable linked, too.

In another embodiment thereof the expression cassette does not comprise an additional transit peptide in case in Table I, application no. 3, in column 6 in the same line as SEQ ID NO. 45897 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 46405, or a homolog or fragment thereof, or a homolog thereof being depicted in Table I, application no. 3, column 8, in the same line as SEQ ID NO. 46405, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 46405 is depicted, plastidic is mentioned, or a mitochondrial transit peptide in case in Table I, application no. 3, in column 6 in the same line as SEQ ID NO. 46405 is depicted, mitochondrial is mentioned; which is operable linked, too.

In another embodiment thereof the expression cassette does not comprise an additional transit peptide in case in Table I, application no. 3, in column 6 in the same line as SEQ ID NO. 46405 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 46515, or a homolog or fragment thereof, or a homolog thereof being depicted in Table I, application no. 3, column 8, in the same line as SEQ ID NO. 46515, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 46515 is depicted, plastidic is mentioned, or a mitochondrial transit peptide in case in Table I, application no. 3, in column 6 in the same line as SEQ ID NO. 46515 is depicted, mitochondrial is mentioned; which is operable linked, too.

In another embodiment thereof the expression cassette does not comprise an additional transit peptide in case in Table I, application no. 3, in column 6 in the same line as SEQ ID NO. 46515 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 84198, or a homolog or fragment thereof, or a homolog thereof being depicted in Table I, application no. 3, column 8, in the same line as SEQ ID NO. 84198, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 84198 is depicted, plastidic is mentioned, or a mitochondrial transit peptide in case in Table I, application no. 3, in column 6 in the same line as SEQ ID NO. 84198 is depicted, mitochondrial is mentioned; which is operable linked, too.

In another embodiment thereof the expression cassette does not comprise an additional transit peptide in case in Table I, application no. 3, in column 6 in the same line as SEQ ID NO. 84198 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 46850, or a homolog or fragment thereof, or a homolog thereof being depicted in Table I, application no. 3, column 8, in the same line as SEQ ID NO. 46850, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 46850 is depicted, plastidic is mentioned, or a mitochondrial transit peptide in case in Table I, application no. 3, in column 6 in the same line as SEQ ID NO. 46850 is depicted, mitochondrial is mentioned; which is operable linked, too.

In another embodiment thereof the expression cassette does not comprise an additional transit peptide in case in Table I, application no. 3, in column 6 in the same line as SEQ ID NO. 46850 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 47159, or a homolog or fragment thereof, or a homolog thereof being depicted in Table I, application no. 3, column 8, in the same line as SEQ ID NO. 47159, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 47159 is depicted, plastidic is mentioned, or a mitochondrial transit peptide in case in Table I, application no. 3, in column 6 in the same line as SEQ ID NO. 47159 is depicted, mitochondrial is mentioned; which is operable linked, too.

In another embodiment thereof the expression cassette does not comprise an additional transit peptide in case in Table I, application no. 3, in column 6 in the same line as SEQ ID NO. 47159 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 59851, or a homolog or fragment thereof, or a homolog thereof being depicted in Table I, application no. 3, column 8, in the same line as SEQ ID NO. 59851, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 59851 is depicted, plastidic is mentioned, or a mitochondrial transit peptide in case in Table I, application no. 3, in column 6 in the same line as SEQ ID NO. 59851 is depicted, mitochondrial is mentioned; which is operable linked, too.

In another embodiment thereof the expression cassette does not comprise an additional transit peptide in case in Table I, application no. 3, in column 6 in the same line as SEQ ID NO. 59851 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 47566, or a homolog or fragment thereof, or a homolog thereof being depicted in Table I, application no. 3, column 8, in the same line as SEQ ID NO. 47566, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 47566 is depicted, plastidic is mentioned, or a mitochondrial transit peptide in case in Table I, application no. 3, in column 6 in the same line as SEQ ID NO. 47566 is depicted, mitochondrial is mentioned; which is operable linked, too.

In another embodiment thereof the expression cassette does not comprise an additional transit peptide in case in Table I, application no. 3, in column 6 in the same line as SEQ ID NO. 47566 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 84265, or a homolog or fragment thereof, or a homolog thereof being depicted in Table I, application no. 3, column 8, in the same line as SEQ ID NO. 84265, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 84265 is depicted, plastidic is mentioned, or a mitochondrial transit peptide in case in Table I, application no. 3, in column 6 in the same line as SEQ ID NO. 84265 is depicted, mitochondrial is mentioned; which is operable linked, too.

In another embodiment thereof the expression cassette does not comprise an additional transit peptide in case in Table I, application no. 3, in column 6 in the same line as SEQ ID NO. 84265 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 84441, or a homolog or fragment thereof, or a homolog thereof being depicted in Table I, application no. 3, column 8, in the same line as SEQ ID NO. 84441, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 84441 is depicted, plastidic is mentioned, or a mitochondrial transit peptide in case in Table I, application no. 3, in column 6 in the same line as SEQ ID NO. 84441 is depicted, mitochondrial is mentioned; which is operable linked, too.

In another embodiment thereof the expression cassette does not comprise an additional transit peptide in case in Table I, application no. 3, in column 6 in the same line as SEQ ID NO. 84441 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 49143, or a homolog or fragment thereof, or a homolog thereof being depicted in Table I, application no. 3, column 8, in the same line as SEQ ID NO. 49143, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 49143 is depicted, plastidic is mentioned, or a mitochondrial transit peptide in case in Table I, application no. 3, in column 6 in the same line as SEQ ID NO. 49143 is depicted, mitochondrial is mentioned; which is operable linked, too.

In another embodiment thereof the expression cassette does not comprise an additional transit peptide in case in Table I, application no. 3, in column 6 in the same line as SEQ ID NO. 49143 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 84865, or a homolog or fragment thereof, or a homolog thereof being depicted in Table I, application no. 3, column 8, in the same line as SEQ ID NO. 84865, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 84865 is depicted, plastidic is mentioned, or a mitochondrial transit peptide in case in Table I, application no. 3, in column 6 in the same line as SEQ ID NO. 84865 is depicted, mitochondrial is mentioned; which is operable linked, too.

In another embodiment thereof the expression cassette does not comprise an additional transit peptide in case in Table I, application no. 3, in column 6 in the same line as SEQ ID NO. 84865 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 84869, or a homolog or fragment thereof, or a homolog thereof being depicted in Table I, application no. 3, column 8, in the same line as SEQ ID NO. 84869, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 84869 is depicted, plastidic is mentioned, or a mitochondrial transit peptide in case in Table I, application no. 3, in column 6 in the same line as SEQ ID NO. 84869 is depicted, mitochondrial is mentioned; which is operable linked, too.

In another embodiment thereof the expression cassette does not comprise an additional transit peptide in case in Table I, application no. 3, in column 6 in the same line as SEQ ID NO. 84869 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 50104, or a homolog or fragment thereof, or a homolog thereof being depicted in Table I, application no. 3, column 8, in the same line as SEQ ID NO. 50104, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 50104 is depicted, plastidic is mentioned, or a mitochondrial transit peptide in case in Table I, application no. 3, in column 6 in the same line as SEQ ID NO. 50104 is depicted, mitochondrial is mentioned; which is operable linked, too.

In another embodiment thereof the expression cassette does not comprise an additional transit peptide in case in Table I, application no. 3, in column 6 in the same line as SEQ ID NO. 50104 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 85510, or a homolog or fragment thereof, or a homolog thereof being depicted in Table I, application no. 3, column 8, in the same line as SEQ ID NO. 85510, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 85510 is depicted, plastidic is mentioned, or a mitochondrial transit peptide in case in Table I, application no. 3, in column 6 in the same line as SEQ ID NO. 85510 is depicted, mitochondrial is mentioned; which is operable linked, too.

In another embodiment thereof the expression cassette does not comprise an additional transit peptide in case in Table I, application no. 3, in column 6 in the same line as SEQ ID NO. 85510 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 50950, or a homolog or fragment thereof, or a homolog thereof being depicted in Table I, application no. 3, column 8, in the same line as SEQ ID NO. 50950, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 50950 is depicted, plastidic is mentioned, or a mitochondrial transit peptide in case in Table I, application no. 3, in column 6 in the same line as SEQ ID NO. 50950 is depicted, mitochondrial is mentioned; which is operable linked, too.

In another embodiment thereof the expression cassette does not comprise an additional transit peptide in case in Table I, application no. 3, in column 6 in the same line as SEQ ID NO. 50950 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 51198, or a homolog or fragment thereof, or a homolog thereof being depicted in Table I, application no. 3, column 8, in the same line as SEQ ID NO. 51198, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 51198 is depicted, plastidic is mentioned, or a mitochondrial transit peptide in case in Table I, application no. 3, in column 6 in the same line as SEQ ID NO. 51198 is depicted, mitochondrial is mentioned; which is operable linked, too.

In another embodiment thereof the expression cassette does not comprise an additional transit peptide in case in Table I, application no. 3, in column 6 in the same line as SEQ ID NO. 51198 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 51268, or a homolog or fragment thereof, or a homolog thereof being depicted in Table I, application no. 3, column 8, in the same line as SEQ ID NO. 51268, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 51268 is depicted, plastidic is mentioned, or a mitochondrial transit peptide in case in Table I, application no. 3, in column 6 in the same line as SEQ ID NO. 51268 is depicted, mitochondrial is mentioned; which is operable linked, too.

In another embodiment thereof the expression cassette does not comprise an additional transit peptide in case in Table I, application no. 3, in column 6 in the same line as SEQ ID NO. 51268 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 52246, or a homolog or fragment thereof, or a homolog thereof being depicted in Table I, application no. 3, column 8, in the same line as SEQ ID NO. 52246, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 52246 is depicted, plastidic is mentioned, or a mitochondrial transit peptide in case in Table I, application no. 3, in column 6 in the same line as SEQ ID NO. 52246 is depicted, mitochondrial is mentioned; which is operable linked, too.

In another embodiment thereof the expression cassette does not comprise an additional transit peptide in case in Table I, application no. 3, in column 6 in the same line as SEQ ID NO. 52246 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 52364, or a homolog or fragment thereof, or a homolog thereof being depicted in Table I, application no. 3, column 8, in the same line as SEQ ID NO. 52364, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 52364 is depicted, plastidic is mentioned, or a mitochondrial transit peptide in case in Table I, application no. 3, in column 6 in the same line as SEQ ID NO. 52364 is depicted, mitochondrial is mentioned; which is operable linked, too.

In another embodiment thereof the expression cassette does not comprise an additional transit peptide in case in Table I, application no. 3, in column 6 in the same line as SEQ ID NO. 52364 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 52634, or a homolog or fragment thereof, or a homolog thereof being depicted in Table I, application no. 3, column 8, in the same line as SEQ ID NO. 52634, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 52634 is depicted, plastidic is mentioned, or a mitochondrial transit peptide in case in Table I, application no. 3, in column 6 in the same line as SEQ ID NO. 52634 is depicted, mitochondrial is mentioned; which is operable linked, too.

In another embodiment thereof the expression cassette does not comprise an additional transit peptide in case in Table I, application no. 3, in column 6 in the same line as SEQ ID NO. 52634 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 85723, or a homolog or fragment thereof, or a homolog thereof being depicted in Table I, application no. 3, column 8, in the same line as SEQ ID NO. 85723, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 85723 is depicted, plastidic is mentioned, or a mitochondrial transit peptide in case in Table I, application no. 3, in column 6 in the same line as SEQ ID NO. 85723 is depicted, mitochondrial is mentioned; which is operable linked, too.

In another embodiment thereof the expression cassette does not comprise an additional transit peptide in case in Table I, application no. 3, in column 6 in the same line as SEQ ID NO. 85723 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 52660, or a homolog or fragment thereof, or a homolog thereof being depicted in Table I, application no. 3, column 8, in the same line as SEQ ID NO. 52660, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 52660 is depicted, plastidic is mentioned, or a mitochondrial transit peptide in case in Table I, application no. 3, in column 6 in the same line as SEQ ID NO. 52660 is depicted, mitochondrial is mentioned; which is operable linked, too.

In another embodiment thereof the expression cassette does not comprise an additional transit peptide in case in Table I, application no. 3, in column 6 in the same line as SEQ ID NO. 52660 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 11211, or a homolog or fragment thereof, or a homolog thereof being depicted in Table I, application no. 3, column 8, in the same line as SEQ ID NO. 11211, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 11211 is depicted, plastidic is mentioned, or a mitochondrial transit peptide in case in Table I, application no. 3, in column 6 in the same line as SEQ ID NO. 11211 is depicted, mitochondrial is mentioned; which is operable linked, too.

In another embodiment thereof the expression cassette does not comprise an additional transit peptide in case in Table I, application no. 3, in column 6 in the same line as SEQ ID NO. 11211 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 53456, or a homolog or fragment thereof, or a homolog thereof being depicted in Table I, application no. 3, column 8, in the same line as SEQ ID NO. 53456, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 53456 is depicted, plastidic is mentioned, or a mitochondrial transit peptide in case in Table I, application no. 3, in column 6 in the same line as SEQ ID NO. 53456 is depicted, mitochondrial is mentioned; which is operable linked, too.

In another embodiment thereof the expression cassette does not comprise an additional transit peptide in case in Table I, application no. 3, in column 6 in the same line as SEQ ID NO. 53456 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 53878, or a homolog or fragment thereof, or a homolog thereof being depicted in Table I, application no. 3, column 8, in the same line as SEQ ID NO. 53878, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 53878 is depicted, plastidic is mentioned, or a mitochondrial transit peptide in case in Table I, application no. 3, in column 6 in the same line as SEQ ID NO. 53878 is depicted, mitochondrial is mentioned; which is operable linked, too.

In another embodiment thereof the expression cassette does not comprise an additional transit peptide in case in Table I, application no. 3, in column 6 in the same line as SEQ ID NO. 53878 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 54337, or a homolog or fragment thereof, or a homolog thereof being depicted in Table I, application no. 3, column 8, in the same line as SEQ ID NO. 54337, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 54337 is depicted, plastidic is mentioned, or a mitochondrial transit peptide in case in Table I, application no. 3, in column 6 in the same line as SEQ ID NO. 54337 is depicted, mitochondrial is mentioned; which is operable linked, too.

In another embodiment thereof the expression cassette does not comprise an additional transit peptide in case in Table I, application no. 3, in column 6 in the same line as SEQ ID NO. 54337 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 54452, or a homolog or fragment thereof, or a homolog thereof being depicted in Table I, application no. 3, column 8, in the same line as SEQ ID NO. 54452, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 54452 is depicted, plastidic is mentioned, or a mitochondrial transit peptide in case in Table I, application no. 3, in column 6 in the same line as SEQ ID NO. 54452 is depicted, mitochondrial is mentioned; which is operable linked, too.

In another embodiment thereof the expression cassette does not comprise an additional transit peptide in case in Table I, application no. 3, in column 6 in the same line as SEQ ID NO. 54452 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 11423, or a homolog or fragment thereof, or a homolog thereof being depicted in Table I, application no. 3, column 8, in the same line as SEQ ID NO. 11423, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 11423 is depicted, plastidic is mentioned, or a mitochondrial transit peptide in case in Table I, application no. 3, in column 6 in the same line as SEQ ID NO. 11423 is depicted, mitochondrial is mentioned; which is operable linked, too.

In another embodiment thereof the expression cassette does not comprise an additional transit peptide in case in Table I, application no. 3, in column 6 in the same line as SEQ ID NO. 11423 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 54804, or a homolog or fragment thereof, or a homolog thereof being depicted in Table I, application no. 3, column 8, in the same line as SEQ ID NO. 54804, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 54804 is depicted, plastidic is mentioned, or a mitochondrial transit peptide in case in Table I, application no. 3, in column 6 in the same line as SEQ ID NO. 54804 is depicted, mitochondrial is mentioned; which is operable linked, too.

In another embodiment thereof the expression cassette does not comprise an additional transit peptide in case in Table I, application no. 3, in column 6 in the same line as SEQ ID NO. 54804 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 85743, or a homolog or fragment thereof, or a homolog thereof being depicted in Table I, application no. 3, column 8, in the same line as SEQ ID NO. 85743, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 85743 is depicted, plastidic is mentioned, or a mitochondrial transit peptide in case in Table I, application no. 3, in column 6 in the same line as SEQ ID NO. 85743 is depicted, mitochondrial is mentioned; which is operable linked, too.

In another embodiment thereof the expression cassette does not comprise an additional transit peptide in case in Table I, application no. 3, in column 6 in the same line as SEQ ID NO. 85743 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 55379, or a homolog or fragment thereof, or a homolog thereof being depicted in Table I, application no. 3, column 8, in the same line as SEQ ID NO. 55379, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 55379 is depicted, plastidic is mentioned, or a mitochondrial transit peptide in case in Table I, application no. 3, in column 6 in the same line as SEQ ID NO. 55379 is depicted, mitochondrial is mentioned; which is operable linked, too.

In another embodiment thereof the expression cassette does not comprise an additional transit peptide in case in Table I, application no. 3, in column 6 in the same line as SEQ ID NO. 55379 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 85808, or a homolog or fragment thereof, or a homolog thereof being depicted in Table I, application no. 3, column 8, in the same line as SEQ ID NO. 85808, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 85808 is depicted, plastidic is mentioned, or a mitochondrial transit peptide in case in Table I, application no. 3, in column 6 in the same line as SEQ ID NO. 85808 is depicted, mitochondrial is mentioned; which is operable linked, too.

In another embodiment thereof the expression cassette does not comprise an additional transit peptide in case in Table I, application no. 3, in column 6 in the same line as SEQ ID NO. 85808 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 86447, or a homolog or fragment thereof, or a homolog thereof being depicted in Table I, application no. 3, column 8, in the same line as SEQ ID NO. 86447, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 86447 is depicted, plastidic is mentioned, or a mitochondrial transit peptide in case in Table I, application no. 3, in column 6 in the same line as SEQ ID NO. 86447 is depicted, mitochondrial is mentioned; which is operable linked, too.

In another embodiment thereof the expression cassette does not comprise an additional transit peptide in case in Table I, application no. 3, in column 6 in the same line as SEQ ID NO. 86447 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 11471, or a homolog or fragment thereof, or a homolog thereof being depicted in Table I, application no. 3, column 8, in the same line as SEQ ID NO. 11471, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 11471 is depicted, plastidic is mentioned, or a mitochondrial transit peptide in case in Table I, application no. 3, in column 6 in the same line as SEQ ID NO. 11471 is depicted, mitochondrial is mentioned; which is operable linked, too.

In another embodiment thereof the expression cassette does not comprise an additional transit peptide in case in Table I, application no. 3, in column 6 in the same line as SEQ ID NO. 11471 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 55385, or a homolog or fragment thereof, or a homolog thereof being depicted in Table I, application no. 3, column 8, in the same line as SEQ ID NO. 55385, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 55385 is depicted, plastidic is mentioned, or a mitochondrial transit peptide in case in Table I, application no. 3, in column 6 in the same line as SEQ ID NO. 55385 is depicted, mitochondrial is mentioned; which is operable linked, too.

In another embodiment thereof the expression cassette does not comprise an additional transit peptide in case in Table I, application no. 3, in column 6 in the same line as SEQ ID NO. 55385 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 86508, or a homolog or fragment thereof, or a homolog thereof being depicted in Table I, application no. 3, column 8, in the same line as SEQ ID NO. 86508, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 86508 is depicted, plastidic is mentioned, or a mitochondrial transit peptide in case in Table I, application no. 3, in column 6 in the same line as SEQ ID NO. 86508 is depicted, mitochondrial is mentioned; which is operable linked, too.

In another embodiment thereof the expression cassette does not comprise an additional transit peptide in case in Table I, application no. 3, in column 6 in the same line as SEQ ID NO. 86508 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 86540, or a homolog or fragment thereof, or a homolog thereof being depicted in Table I, application no. 3, column 8, in the same line as SEQ ID NO. 86540, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 86540 is depicted, plastidic is mentioned, or a mitochondrial transit peptide in case in Table I, application no. 3, in column 6 in the same line as SEQ ID NO. 86540 is depicted, mitochondrial is mentioned; which is operable linked, too.

In another embodiment thereof the expression cassette does not comprise an additional transit peptide in case in Table I, application no. 3, in column 6 in the same line as SEQ ID NO. 86540 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 86820, or a homolog or fragment thereof, or a homolog thereof being depicted in Table I, application no. 3, column 8, in the same line as SEQ ID NO. 86820, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 86820 is depicted, plastidic is mentioned, or a mitochondrial transit peptide in case in Table I, application no. 3, in column 6 in the same line as SEQ ID NO. 86820 is depicted, mitochondrial is mentioned; which is operable linked, too.

In another embodiment thereof the expression cassette does not comprise an additional transit peptide in case in Table I, application no. 3, in column 6 in the same line as SEQ ID NO. 86820 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 56153, or a homolog or fragment thereof, or a homolog thereof being depicted in Table I, application no. 3, column 8, in the same line as SEQ ID NO. 56153, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 56153 is depicted, plastidic is mentioned, or a mitochondrial transit peptide in case in Table I, application no. 3, in column 6 in the same line as SEQ ID NO. 56153 is depicted, mitochondrial is mentioned; which is operable linked, too.

In another embodiment thereof the expression cassette does not comprise an additional transit peptide in case in Table I, application no. 3, in column 6 in the same line as SEQ ID NO. 56153 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 56894, or a homolog or fragment thereof, or a homolog thereof being depicted in Table I, application no. 3, column 8, in the same line as SEQ ID NO. 56894, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 56894 is depicted, plastidic is mentioned, or a mitochondrial transit peptide in case in Table I, application no. 3, in column 6 in the same line as SEQ ID NO. 56894 is depicted, mitochondrial is mentioned; which is operable linked, too.

In another embodiment thereof the expression cassette does not comprise an additional transit peptide in case in Table I, application no. 3, in column 6 in the same line as SEQ ID NO. 56894 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 87397, or a homolog or fragment thereof, or a homolog thereof being depicted in Table I, application no. 3, column 8, in the same line as SEQ ID NO. 87397, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 87397 is depicted, plastidic is mentioned, or a mitochondrial transit peptide in case in Table I, application no. 3, in column 6 in the same line as SEQ ID NO. 87397 is depicted, mitochondrial is mentioned; which is operable linked, too.

In another embodiment thereof the expression cassette does not comprise an additional transit peptide in case in Table I, application no. 3, in column 6 in the same line as SEQ ID NO. 87397 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 57235, or a homolog or fragment thereof, or a homolog thereof being depicted in Table I, application no. 3, column 8, in the same line as SEQ ID NO. 57235, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 57235 is depicted, plastidic is mentioned, or a mitochondrial transit peptide in case in Table I, application no. 3, in column 6 in the same line as SEQ ID NO. 57235 is depicted, mitochondrial is mentioned; which is operable linked, too.

In another embodiment thereof the expression cassette does not comprise an additional transit peptide in case in Table I, application no. 3, in column 6 in the same line as SEQ ID NO. 57235 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 57679, or a homolog or fragment thereof, or a homolog thereof being depicted in Table I, application no. 3, column 8, in the same line as SEQ ID NO. 57679, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 57679 is depicted, plastidic is mentioned, or a mitochondrial transit peptide in case in Table I, application no. 3, in column 6 in the same line as SEQ ID NO. 57679 is depicted, mitochondrial is mentioned; which is operable linked, too.

In another embodiment thereof the expression cassette does not comprise an additional transit peptide in case in Table I, application no. 3, in column 6 in the same line as SEQ ID NO. 57679 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 87610, or a homolog or fragment thereof, or a homolog thereof being depicted in Table I, application no. 3, column 8, in the same line as SEQ ID NO. 87610, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 87610 is depicted, plastidic is mentioned, or a mitochondrial transit peptide in case in Table I, application no. 3, in column 6 in the same line as SEQ ID NO. 87610 is depicted, mitochondrial is mentioned; which is operable linked, too.

In another embodiment thereof the expression cassette does not comprise an additional transit peptide in case in Table I, application no. 3, in column 6 in the same line as SEQ ID NO. 87610 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 87655, or a homolog or fragment thereof, or a homolog thereof being depicted in Table I, application no. 3, column 8, in the same line as SEQ ID NO. 87655, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 87655 is depicted, plastidic is mentioned, or a mitochondrial transit peptide in case in Table I, application no. 3, in column 6 in the same line as SEQ ID NO. 87655 is depicted, mitochondrial is mentioned; which is operable linked, too.

In another embodiment thereof the expression cassette does not comprise an additional transit peptide in case in Table I, application no. 3, in column 6 in the same line as SEQ ID NO. 87655 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 87853, or a homolog or fragment thereof, or a homolog thereof being depicted in Table I, application no. 3, column 8, in the same line as SEQ ID NO. 87853, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 87853 is depicted, plastidic is mentioned, or a mitochondrial transit peptide in case in Table I, application no. 3, in column 6 in the same line as SEQ ID NO. 87853 is depicted, mitochondrial is mentioned; which is operable linked, too.

In another embodiment thereof the expression cassette does not comprise an additional transit peptide in case in Table I, application no. 3, in column 6 in the same line as SEQ ID NO. 87853 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 58058, or a homolog or fragment thereof, or a homolog thereof being depicted in Table I, application no. 3, column 8, in the same line as SEQ ID NO. 58058, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 58058 is depicted, plastidic is mentioned, or a mitochondrial transit peptide in case in Table I, application no. 3, in column 6 in the same line as SEQ ID NO. 58058 is depicted, mitochondrial is mentioned; which is operable linked, too.

In another embodiment thereof the expression cassette does not comprise an additional transit peptide in case in Table I, application no. 3, in column 6 in the same line as SEQ ID NO. 58058 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 58668, or a homolog or fragment thereof, or a homolog thereof being depicted in Table I, application no. 3, column 8, in the same line as SEQ ID NO. 58668, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 58668 is depicted, plastidic is mentioned, or a mitochondrial transit peptide in case in Table I, application no. 3, in column 6 in the same line as SEQ ID NO. 58668 is depicted, mitochondrial is mentioned; which is operable linked, too.

In another embodiment thereof the expression cassette does not comprise an additional transit peptide in case in Table I, application no. 3, in column 6 in the same line as SEQ ID NO. 58668 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 88158, or a homolog or fragment thereof, or a homolog thereof being depicted in Table I, application no. 3, column 8, in the same line as SEQ ID NO. 88158, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 88158 is depicted, plastidic is mentioned, or a mitochondrial transit peptide in case in Table I, application no. 3, in column 6 in the same line as SEQ ID NO. 88158 is depicted, mitochondrial is mentioned; which is operable linked, too.

In another embodiment thereof the expression cassette does not comprise an additional transit peptide in case in Table I, application no. 3, in column 6 in the same line as SEQ ID NO. 88158 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 88271, or a homolog or fragment thereof, or a homolog thereof being depicted in Table I, application no. 3, column 8, in the same line as SEQ ID NO. 88271, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 88271 is depicted, plastidic is mentioned, or a mitochondrial transit peptide in case in Table I, application no. 3, in column 6 in the same line as SEQ ID NO. 88271 is depicted, mitochondrial is mentioned; which is operable linked, too.

In another embodiment thereof the expression cassette does not comprise an additional transit peptide in case in Table I, application no. 3, in column 6 in the same line as SEQ ID NO. 88271 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12070, or a homolog or fragment thereof, or a homolog thereof being depicted in Table I, application no. 3, column 8, in the same line as SEQ ID NO. 12070, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 12070 is depicted, plastidic is mentioned, or a mitochondrial transit peptide in case in Table I, application no. 3, in column 6 in the same line as SEQ ID NO. 12070 is depicted, mitochondrial is mentioned; which is operable linked, too.

In another embodiment thereof the expression cassette does not comprise an additional transit peptide in case in Table I, application no. 3, in column 6 in the same line as SEQ ID NO. 12070 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 58731, or a homolog or fragment thereof, or a homolog thereof being depicted in Table I, application no. 3, column 8, in the same line as SEQ ID NO. 58731, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 58731 is depicted, plastidic is mentioned, or a mitochondrial transit peptide in case in Table I, application no. 3, in column 6 in the same line as SEQ ID NO. 58731 is depicted, mitochondrial is mentioned; which is operable linked, too.

In another embodiment thereof the expression cassette does not comprise an additional transit peptide in case in Table I, application no. 3, in column 6 in the same line as SEQ ID NO. 58731 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 58751, or a homolog or fragment thereof, or a homolog thereof being depicted in Table I, application no. 3, column 8, in the same line as SEQ ID NO. 58751, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 58751 is depicted, plastidic is mentioned, or a mitochondrial transit peptide in case in Table I, application no. 3, in column 6 in the same line as SEQ ID NO. 58751 is depicted, mitochondrial is mentioned; which is operable linked, too.

In another embodiment thereof the expression cassette does not comprise an additional transit peptide in case in Table I, application no. 3, in column 6 in the same line as SEQ ID NO. 58751 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12140, or a homolog or fragment thereof, or a homolog thereof being depicted in Table I, application no. 3, column 8, in the same line as SEQ ID NO. 12140, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 12140 is depicted, plastidic is mentioned, or a mitochondrial transit peptide in case in Table I, application no. 3, in column 6 in the same line as SEQ ID NO. 12140 is depicted, mitochondrial is mentioned; which is operable linked, too.

In another embodiment thereof the expression cassette does not comprise an additional transit peptide in case in Table I, application no. 3, in column 6 in the same line as SEQ ID NO. 12140 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 59041, or a homolog or fragment thereof, or a homolog thereof being depicted in Table I, application no. 3, column 8, in the same line as SEQ ID NO. 59041, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 59041 is depicted, plastidic is mentioned, or a mitochondrial transit peptide in case in Table I, application no. 3, in column 6 in the same line as SEQ ID NO. 59041 is depicted, mitochondrial is mentioned; which is operable linked, too.

In another embodiment thereof the expression cassette does not comprise an additional transit peptide in case in Table I, application no. 3, in column 6 in the same line as SEQ ID NO. 59041 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12341, or a homolog or fragment thereof, or a homolog thereof being depicted in Table I, application no. 3, column 8, in the same line as SEQ ID NO. 12341, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 12341 is depicted, plastidic is mentioned, or a mitochondrial transit peptide in case in Table I, application no. 3, in column 6 in the same line as SEQ ID NO. 12341 is depicted, mitochondrial is mentioned; which is operable linked, too.

In another embodiment thereof the expression cassette does not comprise an additional transit peptide in case in Table I, application no. 3, in column 6 in the same line as SEQ ID NO. 12341 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 59370, or a homolog or fragment thereof, or a homolog thereof being depicted in Table I, application no. 3, column 8, in the same line as SEQ ID NO. 59370, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 59370 is depicted, plastidic is mentioned, or a mitochondrial transit peptide in case in Table I, application no. 3, in column 6 in the same line as SEQ ID NO. 59370 is depicted, mitochondrial is mentioned; which is operable linked, too.

In another embodiment thereof the expression cassette does not comprise an additional transit peptide in case in Table I, application no. 3, in column 6 in the same line as SEQ ID NO. 59370 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12698, or a homolog or fragment thereof, or a homolog thereof being depicted in Table I, application no. 3, column 8, in the same line as SEQ ID NO. 12698, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 12698 is depicted, plastidic is mentioned, or a mitochondrial transit peptide in case in Table I, application no. 3, in column 6 in the same line as SEQ ID NO. 12698 is depicted, mitochondrial is mentioned; which is operable linked, too.

In another embodiment thereof the expression cassette does not comprise an additional transit peptide in case in Table I, application no. 3, in column 6 in the same line as SEQ ID NO. 12698 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 88526, or a homolog or fragment thereof, or a homolog thereof being depicted in Table I, application no. 3, column 8, in the same line as SEQ ID NO. 88526, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 88526 is depicted, plastidic is mentioned, or a mitochondrial transit peptide in case in Table I, application no. 3, in column 6 in the same line as SEQ ID NO. 88526 is depicted, mitochondrial is mentioned; which is operable linked, too.

In another embodiment thereof the expression cassette does not comprise an additional transit peptide in case in Table I, application no. 3, in column 6 in the same line as SEQ ID NO. 88526 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 61070, or a homolog or fragment thereof, or a homolog thereof being depicted in Table I, application no. 3, column 8, in the same line as SEQ ID NO. 61070, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 61070 is depicted, plastidic is mentioned, or a mitochondrial transit peptide in case in Table I, application no. 3, in column 6 in the same line as SEQ ID NO. 61070 is depicted, mitochondrial is mentioned; which is operable linked, too.

In another embodiment thereof the expression cassette does not comprise an additional transit peptide in case in Table I, application no. 3, in column 6 in the same line as SEQ ID NO. 61070 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 61532, or a homolog or fragment thereof, or a homolog thereof being depicted in Table I, application no. 3, column 8, in the same line as SEQ ID NO. 61532, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 61532 is depicted, plastidic is mentioned, or a mitochondrial transit peptide in case in Table I, application no. 3, in column 6 in the same line as SEQ ID NO. 61532 is depicted, mitochondrial is mentioned; which is operable linked, too.

In another embodiment thereof the expression cassette does not comprise an additional transit peptide in case in Table I, application no. 3, in column 6 in the same line as SEQ ID NO. 61532 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 88875, or a homolog or fragment thereof, or a homolog thereof being depicted in Table I, application no. 3, column 8, in the same line as SEQ ID NO. 88875, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 88875 is depicted, plastidic is mentioned, or a mitochondrial transit peptide in case in Table I, application no. 3, in column 6 in the same line as SEQ ID NO. 88875 is depicted, mitochondrial is mentioned; which is operable linked, too.

In another embodiment thereof the expression cassette does not comprise an additional transit peptide in case in Table I, application no. 3, in column 6 in the same line as SEQ ID NO. 88875 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 61553, or a homolog or fragment thereof, or a homolog thereof being depicted in Table I, application no. 3, column 8, in the same line as SEQ ID NO. 61553, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 61553 is depicted, plastidic is mentioned, or a mitochondrial transit peptide in case in Table I, application no. 3, in column 6 in the same line as SEQ ID NO. 61553 is depicted, mitochondrial is mentioned; which is operable linked, too.

In another embodiment thereof the expression cassette does not comprise an additional transit peptide in case in Table I, application no. 3, in column 6 in the same line as SEQ ID NO. 61553 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 61723, or a homolog or fragment thereof, or a homolog thereof being depicted in Table I, application no. 3, column 8, in the same line as SEQ ID NO. 61723, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 61723 is depicted, plastidic is mentioned, or a mitochondrial transit peptide in case in Table I, application no. 3, in column 6 in the same line as SEQ ID NO. 61723 is depicted, mitochondrial is mentioned; which is operable linked, too.

In another embodiment thereof the expression cassette does not comprise an additional transit peptide in case in Table I, application no. 3, in column 6 in the same line as SEQ ID NO. 61723 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 62079, or a homolog or fragment thereof, or a homolog thereof being depicted in Table I, application no. 3, column 8, in the same line as SEQ ID NO. 62079, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 62079 is depicted, plastidic is mentioned, or a mitochondrial transit peptide in case in Table I, application no. 3, in column 6 in the same line as SEQ ID NO. 62079 is depicted, mitochondrial is mentioned; which is operable linked, too.

In another embodiment thereof the expression cassette does not comprise an additional transit peptide in case in Table I, application no. 3, in column 6 in the same line as SEQ ID NO. 62079 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12974, or a homolog or fragment thereof, or a homolog thereof being depicted in Table I, application no. 3, column 8, in the same line as SEQ ID NO. 12974, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 12974 is depicted, plastidic is mentioned, or a mitochondrial transit peptide in case in Table I, application no. 3, in column 6 in the same line as SEQ ID NO. 12974 is depicted, mitochondrial is mentioned; which is operable linked, too.

In another embodiment thereof the expression cassette does not comprise an additional transit peptide in case in Table I, application no. 3, in column 6 in the same line as SEQ ID NO. 12974 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 62160, or a homolog or fragment thereof, or a homolog thereof being depicted in Table I, application no. 3, column 8, in the same line as SEQ ID NO. 62160, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 62160 is depicted, plastidic is mentioned, or a mitochondrial transit peptide in case in Table I, application no. 3, in column 6 in the same line as SEQ ID NO. 62160 is depicted, mitochondrial is mentioned; which is operable linked, too.

In another embodiment thereof the expression cassette does not comprise an additional transit peptide in case in Table I, application no. 3, in column 6 in the same line as SEQ ID NO. 62160 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 62244, or a homolog or fragment thereof, or a homolog thereof being depicted in Table I, application no. 3, column 8, in the same line as SEQ ID NO. 62244, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 62244 is depicted, plastidic is mentioned, or a mitochondrial transit peptide in case in Table I, application no. 3, in column 6 in the same line as SEQ ID NO. 62244 is depicted, mitochondrial is mentioned; which is operable linked, too.

In another embodiment thereof the expression cassette does not comprise an additional transit peptide in case in Table I, application no. 3, in column 6 in the same line as SEQ ID NO. 62244 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 62524, or a homolog or fragment thereof, or a homolog thereof being depicted in Table I, application no. 3, column 8, in the same line as SEQ ID NO. 62524, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 62524 is depicted, plastidic is mentioned, or a mitochondrial transit peptide in case in Table I, application no. 3, in column 6 in the same line as SEQ ID NO. 62524 is depicted, mitochondrial is mentioned; which is operable linked, too.

In another embodiment thereof the expression cassette does not comprise an additional transit peptide in case in Table I, application no. 3, in column 6 in the same line as SEQ ID NO. 62524 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 62717, or a homolog or fragment thereof, or a homolog thereof being depicted in Table I, application no. 3, column 8, in the same line as SEQ ID NO. 62717, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 62717 is depicted, plastidic is mentioned, or a mitochondrial transit peptide in case in Table I, application no. 3, in column 6 in the same line as SEQ ID NO. 62717 is depicted, mitochondrial is mentioned; which is operable linked, too.

In another embodiment thereof the expression cassette does not comprise an additional transit peptide in case in Table I, application no. 3, in column 6 in the same line as SEQ ID NO. 62717 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 88879, or a homolog or fragment thereof, or a homolog thereof being depicted in Table I, application no. 3, column 8, in the same line as SEQ ID NO. 88879, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 88879 is depicted, plastidic is mentioned, or a mitochondrial transit peptide in case in Table I, application no. 3, in column 6 in the same line as SEQ ID NO. 88879 is depicted, mitochondrial is mentioned; which is operable linked, too.

In another embodiment thereof the expression cassette does not comprise an additional transit peptide in case in Table I, application no. 3, in column 6 in the same line as SEQ ID NO. 88879 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 89317, or a homolog or fragment thereof, or a homolog thereof being depicted in Table I, application no. 3, column 8, in the same line as SEQ ID NO. 89317, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 89317 is depicted, plastidic is mentioned, or a mitochondrial transit peptide in case in Table I, application no. 3, in column 6 in the same line as SEQ ID NO. 89317 is depicted, mitochondrial is mentioned; which is operable linked, too.

In another embodiment thereof the expression cassette does not comprise an additional transit peptide in case in Table I, application no. 3, in column 6 in the same line as SEQ ID NO. 89317 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 89395, or a homolog or fragment thereof, or a homolog thereof being depicted in Table I, application no. 3, column 8, in the same line as SEQ ID NO. 89395, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 89395 is depicted, plastidic is mentioned, or a mitochondrial transit peptide in case in Table I, application no. 3, in column 6 in the same line as SEQ ID NO. 89395 is depicted, mitochondrial is mentioned; which is operable linked, too.

In another embodiment thereof the expression cassette does not comprise an additional transit peptide in case in Table I, application no. 3, in column 6 in the same line as SEQ ID NO. 89395 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 14275, or a homolog or fragment thereof, or a homolog thereof being depicted in Table I, application no. 3, column 8, in the same line as SEQ ID NO. 14275, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 14275 is depicted, plastidic is mentioned, or a mitochondrial transit peptide in case in Table I, application no. 3, in column 6 in the same line as SEQ ID NO. 14275 is depicted, mitochondrial is mentioned; which is operable linked, too.

In another embodiment thereof the expression cassette does not comprise an additional transit peptide in case in Table I, application no. 3, in column 6 in the same line as SEQ ID NO. 14275 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 89483, or a homolog or fragment thereof, or a homolog thereof being depicted in Table I, application no. 3, column 8, in the same line as SEQ ID NO. 89483, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 89483 is depicted, plastidic is mentioned, or a mitochondrial transit peptide in case in Table I, application no. 3, in column 6 in the same line as SEQ ID NO. 89483 is depicted, mitochondrial is mentioned; which is operable linked, too.

In another embodiment thereof the expression cassette does not comprise an additional transit peptide in case in Table I, application no. 3, in column 6 in the same line as SEQ ID NO. 89483 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 89936, or a homolog or fragment thereof, or a homolog thereof being depicted in Table I, application no. 3, column 8, in the same line as SEQ ID NO. 89936, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 89936 is depicted, plastidic is mentioned, or a mitochondrial transit peptide in case in Table I, application no. 3, in column 6 in the same line as SEQ ID NO. 89936 is depicted, mitochondrial is mentioned; which is operable linked, too.

In another embodiment thereof the expression cassette does not comprise an additional transit peptide in case in Table I, application no. 3, in column 6 in the same line as SEQ ID NO. 89936 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 89973, or a homolog or fragment thereof, or a homolog thereof being depicted in Table I, application no. 3, column 8, in the same line as SEQ ID NO. 89973, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 89973 is depicted, plastidic is mentioned, or a mitochondrial transit peptide in case in Table I, application no. 3, in column 6 in the same line as SEQ ID NO. 89973 is depicted, mitochondrial is mentioned; which is operable linked, too.

In another embodiment thereof the expression cassette does not comprise an additional transit peptide in case in Table I, application no. 3, in column 6 in the same line as SEQ ID NO. 89973 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 63544, or a homolog or fragment thereof, or a homolog thereof being depicted in Table I, application no. 3, column 8, in the same line as SEQ ID NO. 63544, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 63544 is depicted, plastidic is mentioned, or a mitochondrial transit peptide in case in Table I, application no. 3, in column 6 in the same line as SEQ ID NO. 63544 is depicted, mitochondrial is mentioned; which is operable linked, too.

In another embodiment thereof the expression cassette does not comprise an additional transit peptide in case in Table I, application no. 3, in column 6 in the same line as SEQ ID NO. 63544 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 90103, or a homolog or fragment thereof, or a homolog thereof being depicted in Table I, application no. 3, column 8, in the same line as SEQ ID NO. 90103, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 90103 is depicted, plastidic is mentioned, or a mitochondrial transit peptide in case in Table I, application no. 3, in column 6 in the same line as SEQ ID NO. 90103 is depicted, mitochondrial is mentioned; which is operable linked, too.

In another embodiment thereof the expression cassette does not comprise an additional transit peptide in case in Table I, application no. 3, in column 6 in the same line as SEQ ID NO. 90103 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 90159, or a homolog or fragment thereof, or a homolog thereof being depicted in Table I, application no. 3, column 8, in the same line as SEQ ID NO. 90159, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 90159 is depicted, plastidic is mentioned, or a mitochondrial transit peptide in case in Table I, application no. 3, in column 6 in the same line as SEQ ID NO. 90159 is depicted, mitochondrial is mentioned; which is operable linked, too.

In another embodiment thereof the expression cassette does not comprise an additional transit peptide in case in Table I, application no. 3, in column 6 in the same line as SEQ ID NO. 90159 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 90165, or a homolog or fragment thereof, or a homolog thereof being depicted in Table I, application no. 3, column 8, in the same line as SEQ ID NO. 90165, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 90165 is depicted, plastidic is mentioned, or a mitochondrial transit peptide in case in Table I, application no. 3, in column 6 in the same line as SEQ ID NO. 90165 is depicted, mitochondrial is mentioned; which is operable linked, too.

In another embodiment thereof the expression cassette does not comprise an additional transit peptide in case in Table I, application no. 3, in column 6 in the same line as SEQ ID NO. 90165 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 63745, or a homolog or fragment thereof, or a homolog thereof being depicted in Table I, application no. 3, column 8, in the same line as SEQ ID NO. 63745, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 63745 is depicted, plastidic is mentioned, or a mitochondrial transit peptide in case in Table I, application no. 3, in column 6 in the same line as SEQ ID NO. 63745 is depicted, mitochondrial is mentioned; which is operable linked, too.

In another embodiment thereof the expression cassette does not comprise an additional transit peptide in case in Table I, application no. 3, in column 6 in the same line as SEQ ID NO. 63745 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 63803, or a homolog or fragment thereof, or a homolog thereof being depicted in Table I, application no. 3, column 8, in the same line as SEQ ID NO. 63803, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 63803 is depicted, plastidic is mentioned, or a mitochondrial transit peptide in case in Table I, application no. 3, in column 6 in the same line as SEQ ID NO. 63803 is depicted, mitochondrial is mentioned; which is operable linked, too.

In another embodiment thereof the expression cassette does not comprise an additional transit peptide in case in Table I, application no. 3, in column 6 in the same line as SEQ ID NO. 63803 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 63807, or a homolog or fragment thereof, or a homolog thereof being depicted in Table I, application no. 3, column 8, in the same line as SEQ ID NO. 63807, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 63807 is depicted, plastidic is mentioned, or a mitochondrial transit peptide in case in Table I, application no. 3, in column 6 in the same line as SEQ ID NO. 63807 is depicted, mitochondrial is mentioned; which is operable linked, too.

In another embodiment thereof the expression cassette does not comprise an additional transit peptide in case in Table I, application no. 3, in column 6 in the same line as SEQ ID NO. 63807 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 64144, or a homolog or fragment thereof, or a homolog thereof being depicted in Table I, application no. 3, column 8, in the same line as SEQ ID NO. 64144, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 64144 is depicted, plastidic is mentioned, or a mitochondrial transit peptide in case in Table I, application no. 3, in column 6 in the same line as SEQ ID NO. 64144 is depicted, mitochondrial is mentioned; which is operable linked, too.

In another embodiment thereof the expression cassette does not comprise an additional transit peptide in case in Table I, application no. 3, in column 6 in the same line as SEQ ID NO. 64144 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 90189, or a homolog or fragment thereof, or a homolog thereof being depicted in Table I, application no. 3, column 8, in the same line as SEQ ID NO. 90189, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 90189 is depicted, plastidic is mentioned, or a mitochondrial transit peptide in case in Table I, application no. 3, in column 6 in the same line as SEQ ID NO. 90189 is depicted, mitochondrial is mentioned; which is operable linked, too.

In another embodiment thereof the expression cassette does not comprise an additional transit peptide in case in Table I, application no. 3, in column 6 in the same line as SEQ ID NO. 90189 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 64148, or a homolog or fragment thereof, or a homolog thereof being depicted in Table I, application no. 3, column 8, in the same line as SEQ ID NO. 64148, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 64148 is depicted, plastidic is mentioned, or a mitochondrial transit peptide in case in Table I, application no. 3, in column 6 in the same line as SEQ ID NO. 64148 is depicted, mitochondrial is mentioned; which is operable linked, too.

In another embodiment thereof the expression cassette does not comprise an additional transit peptide in case in Table I, application no. 3, in column 6 in the same line as SEQ ID NO. 64148 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 90278, or a homolog or fragment thereof, or a homolog thereof being depicted in Table I, application no. 3, column 8, in the same line as SEQ ID NO. 90278, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 90278 is depicted, plastidic is mentioned, or a mitochondrial transit peptide in case in Table I, application no. 3, in column 6 in the same line as SEQ ID NO. 90278 is depicted, mitochondrial is mentioned; which is operable linked, too.

In another embodiment thereof the expression cassette does not comprise an additional transit peptide in case in Table I, application no. 3, in column 6 in the same line as SEQ ID NO. 90278 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 64177, or a homolog or fragment thereof, or a homolog thereof being depicted in Table I, application no. 3, column 8, in the same line as SEQ ID NO. 64177, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 64177 is depicted, plastidic is mentioned, or a mitochondrial transit peptide in case in Table I, application no. 3, in column 6 in the same line as SEQ ID NO. 64177 is depicted, mitochondrial is mentioned; which is operable linked, too.

In another embodiment thereof the expression cassette does not comprise an additional transit peptide in case in Table I, application no. 3, in column 6 in the same line as SEQ ID NO. 64177 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 64315, or a homolog or fragment thereof, or a homolog thereof being depicted in Table I, application no. 3, column 8, in the same line as SEQ ID NO. 64315, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 64315 is depicted, plastidic is mentioned, or a mitochondrial transit peptide in case in Table I, application no. 3, in column 6 in the same line as SEQ ID NO. 64315 is depicted, mitochondrial is mentioned; which is operable linked, too.

In another embodiment thereof the expression cassette does not comprise an additional transit peptide in case in Table I, application no. 3, in column 6 in the same line as SEQ ID NO. 64315 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 14715, or a homolog or fragment thereof, or a homolog thereof being depicted in Table I, application no. 3, column 8, in the same line as SEQ ID NO. 14715, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 14715 is depicted, plastidic is mentioned, or a mitochondrial transit peptide in case in Table I, application no. 3, in column 6 in the same line as SEQ ID NO. 14715 is depicted, mitochondrial is mentioned; which is operable linked, too.

In another embodiment thereof the expression cassette does not comprise an additional transit peptide in case in Table I, application no. 3, in column 6 in the same line as SEQ ID NO. 14715 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 90284, or a homolog or fragment thereof, or a homolog thereof being depicted in Table I, application no. 3, column 8, in the same line as SEQ ID NO. 90284, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 90284 is depicted, plastidic is mentioned, or a mitochondrial transit peptide in case in Table I, application no. 3, in column 6 in the same line as SEQ ID NO. 90284 is depicted, mitochondrial is mentioned; which is operable linked, too.

In another embodiment thereof the expression cassette does not comprise an additional transit peptide in case in Table I, application no. 3, in column 6 in the same line as SEQ ID NO. 90284 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 64546, or a homolog or fragment thereof, or a homolog thereof being depicted in Table I, application no. 3, column 8, in the same line as SEQ ID NO. 64546, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 64546 is depicted, plastidic is mentioned, or a mitochondrial transit peptide in case in Table I, application no. 3, in column 6 in the same line as SEQ ID NO. 64546 is depicted, mitochondrial is mentioned; which is operable linked, too.

In another embodiment thereof the expression cassette does not comprise an additional transit peptide in case in Table I, application no. 3, in column 6 in the same line as SEQ ID NO. 64546 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 64563, or a homolog or fragment thereof, or a homolog thereof being depicted in Table I, application no. 3, column 8, in the same line as SEQ ID NO. 64563, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 64563 is depicted, plastidic is mentioned, or a mitochondrial transit peptide in case in Table I, application no. 3, in column 6 in the same line as SEQ ID NO. 64563 is depicted, mitochondrial is mentioned; which is operable linked, too.

In another embodiment thereof the expression cassette does not comprise an additional transit peptide in case in Table I, application no. 3, in column 6 in the same line as SEQ ID NO. 64563 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 90306, or a homolog or fragment thereof, or a homolog thereof being depicted in Table I, application no. 3, column 8, in the same line as SEQ ID NO. 90306, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 90306 is depicted, plastidic is mentioned, or a mitochondrial transit peptide in case in Table I, application no. 3, in column 6 in the same line as SEQ ID NO. 90306 is depicted, mitochondrial is mentioned; which is operable linked, too.

In another embodiment thereof the expression cassette does not comprise an additional transit peptide in case in Table I, application no. 3, in column 6 in the same line as SEQ ID NO. 90306 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 64964, or a homolog or fragment thereof, or a homolog thereof being depicted in Table I, application no. 3, column 8, in the same line as SEQ ID NO. 64964, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 64964 is depicted, plastidic is mentioned, or a mitochondrial transit peptide in case in Table I, application no. 3, in column 6 in the same line as SEQ ID NO. 64964 is depicted, mitochondrial is mentioned; which is operable linked, too.

In another embodiment thereof the expression cassette does not comprise an additional transit peptide in case in Table I, application no. 3, in column 6 in the same line as SEQ ID NO. 64964 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 14843, or a homolog or fragment thereof, or a homolog thereof being depicted in Table I, application no. 3, column 8, in the same line as SEQ ID NO. 14843, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 14843 is depicted, plastidic is mentioned, or a mitochondrial transit peptide in case in Table I, application no. 3, in column 6 in the same line as SEQ ID NO. 14843 is depicted, mitochondrial is mentioned; which is operable linked, too.

In another embodiment thereof the expression cassette does not comprise an additional transit peptide in case in Table I, application no. 3, in column 6 in the same line as SEQ ID NO. 14843 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 90409, or a homolog or fragment thereof, or a homolog thereof being depicted in Table I, application no. 3, column 8, in the same line as SEQ ID NO. 90409, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 90409 is depicted, plastidic is mentioned, or a mitochondrial transit peptide in case in Table I, application no. 3, in column 6 in the same line as SEQ ID NO. 90409 is depicted, mitochondrial is mentioned; which is operable linked, too.

In another embodiment thereof the expression cassette does not comprise an additional transit peptide in case in Table I, application no. 3, in column 6 in the same line as SEQ ID NO. 90409 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 66274, or a homolog or fragment thereof, or a homolog thereof being depicted in Table I, application no. 3, column 8, in the same line as SEQ ID NO. 66274, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 66274 is depicted, plastidic is mentioned, or a mitochondrial transit peptide in case in Table I, application no. 3, in column 6 in the same line as SEQ ID NO. 66274 is depicted, mitochondrial is mentioned; which is operable linked, too.

In another embodiment thereof the expression cassette does not comprise an additional transit peptide in case in Table I, application no. 3, in column 6 in the same line as SEQ ID NO. 66274 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 90602, or a homolog or fragment thereof, or a homolog thereof being depicted in Table I, application no. 3, column 8, in the same line as SEQ ID NO. 90602, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 90602 is depicted, plastidic is mentioned, or a mitochondrial transit peptide in case in Table I, application no. 3, in column 6 in the same line as SEQ ID NO. 90602 is depicted, mitochondrial is mentioned; which is operable linked, too.

In another embodiment thereof the expression cassette does not comprise an additional transit peptide in case in Table I, application no. 3, in column 6 in the same line as SEQ ID NO. 90602 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 90615, or a homolog or fragment thereof, or a homolog thereof being depicted in Table I, application no. 3, column 8, in the same line as SEQ ID NO. 90615, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 90615 is depicted, plastidic is mentioned, or a mitochondrial transit peptide in case in Table I, application no. 3, in column 6 in the same line as SEQ ID NO. 90615 is depicted, mitochondrial is mentioned; which is operable linked, too.

In another embodiment thereof the expression cassette does not comprise an additional transit peptide in case in Table I, application no. 3, in column 6 in the same line as SEQ ID NO. 90615 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 91045, or a homolog or fragment thereof, or a homolog thereof being depicted in Table I, application no. 3, column 8, in the same line as SEQ ID NO. 91045, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 91045 is depicted, plastidic is mentioned, or a mitochondrial transit peptide in case in Table I, application no. 3, in column 6 in the same line as SEQ ID NO. 91045 is depicted, mitochondrial is mentioned; which is operable linked, too.

In another embodiment thereof the expression cassette does not comprise an additional transit peptide in case in Table I, application no. 3, in column 6 in the same line as SEQ ID NO. 91045 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 91659, or a homolog or fragment thereof, or a homolog thereof being depicted in Table I, application no. 3, column 8, in the same line as SEQ ID NO. 91659, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 91659 is depicted, plastidic is mentioned, or a mitochondrial transit peptide in case in Table I, application no. 3, in column 6 in the same line as SEQ ID NO. 91659 is depicted, mitochondrial is mentioned; which is operable linked, too.

In another embodiment thereof the expression cassette does not comprise an additional transit peptide in case in Table I, application no. 3, in column 6 in the same line as SEQ ID NO. 91659 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 91781, or a homolog or fragment thereof, or a homolog thereof being depicted in Table I, application no. 3, column 8, in the same line as SEQ ID NO. 91781, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 91781 is depicted, plastidic is mentioned, or a mitochondrial transit peptide in case in Table I, application no. 3, in column 6 in the same line as SEQ ID NO. 91781 is depicted, mitochondrial is mentioned; which is operable linked, too.

In another embodiment thereof the expression cassette does not comprise an additional transit peptide in case in Table I, application no. 3, in column 6 in the same line as SEQ ID NO. 91781 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 91785, or a homolog or fragment thereof, or a homolog thereof being depicted in Table I, application no. 3, column 8, in the same line as SEQ ID NO. 91785, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 91785 is depicted, plastidic is mentioned, or a mitochondrial transit peptide in case in Table I, application no. 3, in column 6 in the same line as SEQ ID NO. 91785 is depicted, mitochondrial is mentioned; which is operable linked, too.

In another embodiment thereof the expression cassette does not comprise an additional transit peptide in case in Table I, application no. 3, in column 6 in the same line as SEQ ID NO. 91785 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 91958, or a homolog or fragment thereof, or a homolog thereof being depicted in Table I, application no. 3, column 8, in the same line as SEQ ID NO. 91958, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 91958 is depicted, plastidic is mentioned, or a mitochondrial transit peptide in case in Table I, application no. 3, in column 6 in the same line as SEQ ID NO. 91958 is depicted, mitochondrial is mentioned; which is operable linked, too.

In another embodiment thereof the expression cassette does not comprise an additional transit peptide in case in Table I, application no. 3, in column 6 in the same line as SEQ ID NO. 91958 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 91973, or a homolog or fragment thereof, or a homolog thereof being depicted in Table I, application no. 3, column 8, in the same line as SEQ ID NO. 91973, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 91973 is depicted, plastidic is mentioned, or a mitochondrial transit peptide in case in Table I, application no. 3, in column 6 in the same line as SEQ ID NO. 91973 is depicted, mitochondrial is mentioned; which is operable linked, too.

In another embodiment thereof the expression cassette does not comprise an additional transit peptide in case in Table I, application no. 3, in column 6 in the same line as SEQ ID NO. 91973 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 92274, or a homolog or fragment thereof, or a homolog thereof being depicted in Table I, application no. 3, column 8, in the same line as SEQ ID NO. 92274, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 92274 is depicted, plastidic is mentioned, or a mitochondrial transit peptide in case in Table I, application no. 3, in column 6 in the same line as SEQ ID NO. 92274 is depicted, mitochondrial is mentioned; which is operable linked, too.

In another embodiment thereof the expression cassette does not comprise an additional transit peptide in case in Table I, application no. 3, in column 6 in the same line as SEQ ID NO. 92274 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 92325, or a homolog or fragment thereof, or a homolog thereof being depicted in Table I, application no. 3, column 8, in the same line as SEQ ID NO. 92325, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 92325 is depicted, plastidic is mentioned, or a mitochondrial transit peptide in case in Table I, application no. 3, in column 6 in the same line as SEQ ID NO. 92325 is depicted, mitochondrial is mentioned; which is operable linked, too.

In another embodiment thereof the expression cassette does not comprise an additional transit peptide in case in Table I, application no. 3, in column 6 in the same line as SEQ ID NO. 92325 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 92339, or a homolog or fragment thereof, or a homolog thereof being depicted in Table I, application no. 3, column 8, in the same line as SEQ ID NO. 92339, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 92339 is depicted, plastidic is mentioned, or a mitochondrial transit peptide in case in Table I, application no. 3, in column 6 in the same line as SEQ ID NO. 92339 is depicted, mitochondrial is mentioned; which is operable linked, too.

In another embodiment thereof the expression cassette does not comprise an additional transit peptide in case in Table I, application no. 3, in column 6 in the same line as SEQ ID NO. 92339 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 92391, or a homolog or fragment thereof, or a homolog thereof being depicted in Table I, application no. 3, column 8, in the same line as SEQ ID NO. 92391, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 92391 is depicted, plastidic is mentioned, or a mitochondrial transit peptide in case in Table I, application no. 3, in column 6 in the same line as SEQ ID NO. 92391 is depicted, mitochondrial is mentioned; which is operable linked, too.

In another embodiment thereof the expression cassette does not comprise an additional transit peptide in case in Table I, application no. 3, in column 6 in the same line as SEQ ID NO. 92391 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 92510, or a homolog or fragment thereof, or a homolog thereof being depicted in Table I, application no. 3, column 8, in the same line as SEQ ID NO. 92510, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 92510 is depicted, plastidic is mentioned, or a mitochondrial transit peptide in case in Table I, application no. 3, in column 6 in the same line as SEQ ID NO. 92510 is depicted, mitochondrial is mentioned; which is operable linked, too.

In another embodiment thereof the expression cassette does not comprise an additional transit peptide in case in Table I, application no. 3, in column 6 in the same line as SEQ ID NO. 92510 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 15179, or a homolog or fragment thereof, or a homolog thereof being depicted in Table I, application no. 3, column 8, in the same line as SEQ ID NO. 15179, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 15179 is depicted, plastidic is mentioned, or a mitochondrial transit peptide in case in Table I, application no. 3, in column 6 in the same line as SEQ ID NO. 15179 is depicted, mitochondrial is mentioned; which is operable linked, too.

In another embodiment thereof the expression cassette does not comprise an additional transit peptide in case in Table I, application no. 3, in column 6 in the same line as SEQ ID NO. 15179 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 67968, or a homolog or fragment thereof, or a homolog thereof being depicted in Table I, application no. 3, column 8, in the same line as SEQ ID NO. 67968, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 67968 is depicted, plastidic is mentioned, or a mitochondrial transit peptide in case in Table I, application no. 3, in column 6 in the same line as SEQ ID NO. 67968 is depicted, mitochondrial is mentioned; which is operable linked, too.

In another embodiment thereof the expression cassette does not comprise an additional transit peptide in case in Table I, application no. 3, in column 6 in the same line as SEQ ID NO. 67968 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 92540, or a homolog or fragment thereof, or a homolog thereof being depicted in Table I, application no. 3, column 8, in the same line as SEQ ID NO. 92540, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 92540 is depicted, plastidic is mentioned, or a mitochondrial transit peptide in case in Table I, application no. 3, in column 6 in the same line as SEQ ID NO. 92540 is depicted, mitochondrial is mentioned; which is operable linked, too.

In another embodiment thereof the expression cassette does not comprise an additional transit peptide in case in Table I, application no. 3, in column 6 in the same line as SEQ ID NO. 92540 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 92616, or a homolog or fragment thereof, or a homolog thereof being depicted in Table I, application no. 3, column 8, in the same line as SEQ ID NO. 92616, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 92616 is depicted, plastidic is mentioned, or a mitochondrial transit peptide in case in Table I, application no. 3, in column 6 in the same line as SEQ ID NO. 92616 is depicted, mitochondrial is mentioned; which is operable linked, too.

In another embodiment thereof the expression cassette does not comprise an additional transit peptide in case in Table I, application no. 3, in column 6 in the same line as SEQ ID NO. 92616 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 92668, or a homolog or fragment thereof, or a homolog thereof being depicted in Table I, application no. 3, column 8, in the same line as SEQ ID NO. 92668, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 92668 is depicted, plastidic is mentioned, or a mitochondrial transit peptide in case in Table I, application no. 3, in column 6 in the same line as SEQ ID NO. 92668 is depicted, mitochondrial is mentioned; which is operable linked, too.

In another embodiment thereof the expression cassette does not comprise an additional transit peptide in case in Table I, application no. 3, in column 6 in the same line as SEQ ID NO. 92668 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 93091, or a homolog or fragment thereof, or a homolog thereof being depicted in Table I, application no. 3, column 8, in the same line as SEQ ID NO. 93091, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 93091 is depicted, plastidic is mentioned, or a mitochondrial transit peptide in case in Table I, application no. 3, in column 6 in the same line as SEQ ID NO. 93091 is depicted, mitochondrial is mentioned; which is operable linked, too.

In another embodiment thereof the expression cassette does not comprise an additional transit peptide in case in Table I, application no. 3, in column 6 in the same line as SEQ ID NO. 93091 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 93218, or a homolog or fragment thereof, or a homolog thereof being depicted in Table I, application no. 3, column 8, in the same line as SEQ ID NO. 93218, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 93218 is depicted, plastidic is mentioned, or a mitochondrial transit peptide in case in Table I, application no. 3, in column 6 in the same line as SEQ ID NO. 93218 is depicted, mitochondrial is mentioned; which is operable linked, too.

In another embodiment thereof the expression cassette does not comprise an additional transit peptide in case in Table I, application no. 3, in column 6 in the same line as SEQ ID NO. 93218 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68413, or a homolog or fragment thereof, or a homolog thereof being depicted in Table I, application no. 3, column 8, in the same line as SEQ ID NO. 68413, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 3, in column 6 in the same line as SEQ ID NO. 68413 is depicted, plastidic is mentioned, or a mitochondrial transit peptide in case in Table I, application no. 3, in column 6 in the same line as SEQ ID NO. 68413 is depicted, mitochondrial is mentioned; which is operable linked, too.

In another embodiment thereof the expression cassette does not comprise an additional transit peptide in case in Table I, application no. 3, in column 6 in the same line as SEQ ID NO. 68413 is depicted, non-targeted is mentioned.

[0165.1.1.3] to [0170.1.1.3] for the disclosure of these paragraphs see [0165.1.1.1] to [0170.1.1.1] above.

The invention further provides a vector, comprising a nucleic acid molecule comprising 1) a nucleic acid molecule selected from the group consisting of:
(a) a nucleic acid molecule encoding a polypeptide shown in Table II, column 5 or 8, application no. 3, preferably shown in Table II A, application no. 3, in column 5, or in Table II A, application no. 3, column 8, or in Table II B, application no. 3, column 8, or a homolog or a fragment thereof;
(b) a nucleic acid molecule shown in Table I, application no. 3, column 5 or 8, preferably shown in Table I A, application no. 3, in column 5, or in Table I A, application no. 3, column 8, or in Table I B, application no. 3, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 3, preferably shown in Table II A, application no. 3, in column 5, or in Table II A, application no. 3, column 8, or in Table II B, application no. 3, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 3, preferably shown in Table I A, application no. 3, in column 5, or in Table I A, application no. 3, column 8, or in Table I B, application no. 3, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 3;
(i) a nucleic acid molecule encoding a polypeptide having the activity represented by a protein as depicted in column 5 of Table II, application no. 3;
(j) a nucleic acid molecule comprising a polynucleotide which is obtained by amplifying a cDNA library or a genomic library using the primers in Table III, application no. 3, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
   or a nucleic acid molecule comprising a sequence which is complementary thereto.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 3.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 3, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 3.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 3, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 3, in the same line as of the respective nucleic acid molecule according to 1) “plastidic” is depicted, or encoding a mitochondrial transit peptide, especially in case in column 6 of Table I, application no. 3, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 3, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 3, in the same line as of the respective nucleic acid molecule according to 1) “plastidic” is depicted, or encoding a mitochondrial transit peptide, especially in case in column 6 of Table I, application no. 3, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operable linked; a nucleic acid molecule encoding a transit peptide, especially in case in column 6 of Table I, application no. 3, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 3, in the same line as of the respective nucleic acid molecule according to 1) “plastidic” is depicted, or encoding a mitochondrial transit peptide, especially in case in column 6 of Table I, application no. 3, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 3 (in the same line).

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 3, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 3.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 3, in the same line as of the respective nucleic acid molecule according to 1) “plastidic” is depicted, or encoding a mitochondrial transit peptide, especially in case in column 6 of Table I, application no. 3, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 3, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 3, in the same line as of the respective nucleic acid molecule according to 1) “plastidic” is depicted, or encoding a mitochondrial transit peptide, especially in case in column 6 of Table I, application no. 3, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 3 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 3, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 3 (in the same line) and confers a generation or an increase of the respective fine chemical in a non-human organism or a part thereof as compared to a corresponding wild-type non-human organism or part thereof.

In a preferred embodiment of said vector said nucleic acid molecule according to 1 (a), (b), (c), (d), (e), (f), (g), (h), (i), (j) or (k), said promoter and optionally said nucleic acid molecule encoding a transit peptide are encompassed in an expression cassette. Accordingly in an embodiment the invention provides a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

The invention further provides a vector comprising a nucleic acid molecule comprising 1) a nucleic acid molecule selected from the group consisting of:
(a) a nucleic acid molecule encoding a polypeptide shown in Table II, column 5 or 8, application no. 3, preferably shown in Table II A, application no. 3, in column 5, or in Table II A, application no. 3, column 8, or in Table II B, application no. 3, column 8, or a homolog or a fragment thereof;
(b) a nucleic acid molecule shown in Table I, application no. 3, column 5 or 8, preferably shown in Table I A, application no. 3, in column 5, or in Table I A, application no. 3, column 8, or in Table I B, application no. 3, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 3, preferably shown in Table II A, application no. 3, in column 5, or in Table II A, application no. 3, column 8, or in Table II B, application no. 3, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 3, preferably shown in Table I A, application no. 3, in column 5, or in Table I A, application no. 3, column 8, or in Table I B, application no. 3, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 3;
(i) a nucleic acid molecule encoding a polypeptide having the activity represented by a protein as depicted in column 5 of Table II, application no. 3;
(j) a nucleic acid molecule comprising a polynucleotide which is obtained by amplifying a cDNA library or a genomic library using the primers in Table III, application no. 3, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
   or a nucleic acid molecule comprising a sequence which is complementary thereto;
   whereby, preferably, the nucleic acid molecule according to 1 (a), (b), (c), (d), (e), (f), (g), (h), (i), (j) or (k) distinguishes over the sequence depicted in Table I A and/or I B, application no. 3, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 3, column 5 or 8, or the coding region thereof, respectively. In another embodiment, the nucleic acid molecule of the present invention, preferably the coding region thereof, is 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 3, column 5 or 8, or the coding region thereof, respectively. In a further embodiment the nucleic acid molecule does not encode the polypeptide sequence shown in Table II A and/or II B, application no. 3, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 3, column 5 or 8 and does not encode a protein of the sequence shown in Table II A and/or II B, application no. 3, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 3, column 5 and 8, or the coding region thereof. In a further embodiment, the protein of the present invention is at least 30%,40%, 50%, 60%, 70%, 80%, 90%, 95% identical to protein sequence depicted in Table II A and/or II B, application no. 3, column 5 or 8 but less than 100%, preferably less than 99.999%, 99.99% or 99.9%, more preferably less than 99%, 985, 97%, 96% or 95% identical to the sequence shown in Table II A and/or II B, application no. 3, column 5 or 8.

(All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 3.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 3, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 3.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 3, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 3, in the same line as of the respective nucleic acid molecule according to 1) “plastidic” is depicted, or encoding a mitochondrial transit peptide, especially in case in column 6 of Table I, application no. 3, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 3, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 3, in the same line as of the respective nucleic acid molecule according to 1) “plastidic” is depicted, or encoding a mitochondrial transit peptide, especially in case in column 6 of Table I, application no. 3, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 3, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 3, in the same line as of the respective nucleic acid molecule according to 1) “plastidic” is depicted, or encoding a mitochondrial transit peptide, especially in case in column 6 of Table I, application no. 3, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 3.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 3, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 3.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 3, in the same line as of the respective nucleic acid molecule according to 1) “plastidic” is depicted, or encoding a mitochondrial transit peptide, especially in case in column 6 of Table I, application no. 3, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 3, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 3, in the same line as of the respective nucleic acid molecule according to 1) “plastidic” is depicted, or encoding a mitochondrial transit peptide, especially in case in column 6 of Table I, application no. 3, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 3 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 3, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 3 and confers a generation or an increase of the respective fine chemical in a non-human organism or a part thereof as compared to a corresponding wild-type non-human organism or part thereof.

[0181.1.1.3] to [0209.1.1.3] for the disclosure of these paragraphs see [0181.1.1.1] to [0209.1.1.1] above.

The genes of the invention, coding for an activity selected from the group consisting of 2,3-dihydroxyphenylpropionate 1,2-dioxygenase, 2-C-methyl-D-erythritol 4-phosphate cytidylyltransferase, 2-oxoglutarate dehydrogenase E1 subunit, 30S ribosomal protein S3, 3-deoxy-7-phosphoheptulonate synthase, 3-deoxy-D-arabinoheptulosonate-7-phosphatesynthase, 3-oxoacyl-[acyl-carrier-protein] synthase, 3-phosphoglycerate dehydrogenase, 47266012-protein, 49747384_SOYBEAN-protein, 50S ribosomal protein L14, 5′-nucleotidase, ABC metal ion transporter substrate-binding protein, ABC transporter ATP binding component, ABC transporter ATP-binding protein, ABC transporter component, ABC transporter permease protein, ABC transporter substrate-binding protein, acetolactate synthase, acetolactate synthase small subunit, acetyl CoA carboxylase, acetyl-CoA acetyltransferase, acetyltransferase, acid shock protein, acyl transferase, acyl-CoA dehydrogenase, acyl-CoA synthase, adenylate kinase, adenylylsulfate kinase, agmatinase, aldehyde dehydrogenase, amine oxidase, amino acid ABC transporter permease protein, amino acid acetyltransferase, amino acid transporter, amino-acid acetyltransferase, aminotransferase, ammonium transporter, ankyrin repeat family protein, anthranilate synthase component I, arginine exporter protein, arginine N-succinyltransferase, argininosuccinate lyase, asparaginase, aspartate 1-decarboxylase, aspartate aminotransferase, aspartate kinase, aspartate-ammonia ligase, At1g17440-protein, At1g19800-protein, At4g32480-protein, At5g16650-protein, at5g21910-protein, ATP-binding component of a transport system, ATP-dependent RNA helicase, auxin response factor, AX653549-protein, AY087308-protein, b0391-protein, b0456-protein, b0518-protein, b0801-protein, b0841-protein, b0917-protein, b1003-protein, b1024-protein, b1137-protein, b1155-protein, b1163-protein, b1179-protein, b1205-protein, b1229-protein, b1259-protein, b1280-protein, b1330-protein, b1445-protein, b1522-protein, b1583-protein, b1670-protein, b1672-protein, b1837-protein, b1898-protein, b1930-protein, b2032-protein, b2107-protein, b2360-protein, b2399-protein, b2474-protein, b2513-protein, b2613-protein, b2673-protein, b2739-protein, b2812-protein, b2849-protein, b3083-protein, b3098-protein, b3121-protein, b3221-protein, b3246-protein, b3346-protein, b3392-protein, b3410-protein, b3509-protein, b3646-protein, b3791-protein, b3814-protein, b3989-protein, b4029-protein, b4121-protein, beta-galactosidase, betahydroxylase, bifunctional aspartokinase/homoserine dehydrogenase, bifunctional purine biosynthesis protein, branched chain aminotransferase, branched-chain amino acid permease, calcium-dependent protein kinase, carbon dioxide concentrating mechanism protein, cationtransporting ATPase, CBL-interacting protein kinase, CDP-diacylglycerol-glycerol-3-phosphate 3-phosphatidyltransferase, CDP-diacylglycerol-serine O-phosphatidyltransferase, CDP-glucose-4,6-dehydratase, cell division control protein, cellulose synthase catalytic subunit, Chaperone protein CIpB, chorismate mutase-T and prephenate dehydrogenase, citrate synthase, coproporphyrinogen III oxidase, CTP synthetase, cullin, cyclin D, cystathionine gammasynthase, cytochrome c-type protein, D-3-phosphoglycerate dehydrogenase, delta-1-pyrroline 5-carboxylase synthetase, diacylglycerol O-acyltransferase, dihydroxyacid dehydratase, DNA helicase II, DNA mismatch repair protein, DNA-binding protein, DnaJ-like chaperone, D-serine dehydratase, electron transfer flavoprotein subunit beta, elongation factor Tu, enoyl-CoA hydratase, enterobacterial common antigen polymerase, eukaryotic translation initiation factor, eukaryotic translation initiation factor 5, exopolyphosphatase, Fe—S subunit of oxidoreductase, flavodoxin, fumarylacetoacetate hydrolase, gamma-glutamyltranspeptidase, GDP-mannose dehydrogenase, geranylgeranyl pyrophosphate synthase, gibberellin 20-oxidase, Glu/Leu/Phe/Val dehydrogenase , glucokinase, glucose dehydrogenase, glucose-1-phosphate cytidylyltransferase, glucose-6-phosphate 1-dehydrogenase, glutamate-ammonia-ligase, glutamine amidotransferase, glutamine synthetase, glutaredoxin, glutathione peroxidase, glutathione S-transferase, Glycine cleavage system T aminomethyltransferase, glycogen (starch) synthase, glycogen synthase, glycoprotease, glycosyl transferase, GM02LC11114-protein, GM02LC17485-protein, harpin-induced family protein, heat shock transcription factor, HesB/YadR/YfhF family protein, histone acetyltransferase, histone H2A, homocitrate synthase, homoserine O-acetyltransferase, hydrolase, integral membrane transporter family protein, iron(III) dicitrate-binding protein, isochorismate synthase, kinase, L-aspartate oxidase, lipoprotein precursor, lysine decarboxylase, lysyl-tRNA synthetase, major facilitator superfamily transporter protein, malate dehydrogenase, malic enzyme, membrane protein, membrane transport protein, metal ion transporter, metal-dependent hydrolase, methylglyoxal synthase, methylglyoxalase, methyltransferase, mitochondrial inner membrane protease subunit, molecular chaperone portein, monothiol glutaredoxin, monthiol glutaredoxin, multidrug resistance protein, multiple antibiotic resistance protein, murein transglycosylase, N-(5′-phosphoribosyl)anthranilate isomerase, Na+/K+ transporter, N-acetyl-gamma-glutamyl-phosphate reductase, NADH dehydrogenase I chain I, NADH dehydrogenase subunit N, NADH-quinone oxidoreductase subunit, NADP-dependent malic enzyme, nitrate/nitrite transport protein, O-antigen chain length determinant, oxidoreductase, oxidoreductase subunit, oxireductase, peptidase, permease protein of phosphate ABC transporter, phosphate permease, phosphate transporter, phosphoadenosine phosphosulfate reductase , phosphoanhydride phosphorylase, phosphoenolpyruvate carboxykinase, phosphopantetheine adenylyltransferase, phosphoribosylformyl glycinamidine synthase subunit, Photosystem I reaction center subunit XI, photosystem II protein, polygalacturonase, precorrin methylase, Pre-mRNA-splicing factor, prolipoprotein diacylglyceryl transferase, protease, protein kinase, protein phosphatase, PTS system N-acetylmuramic acid-specific EIIBC component, purine biosynthesis protein, pyruvate kinase, restriction alleviation protein, riboflavin biosynthesis protein, RNA binding protein, RNA polymerase sigma factor, RNA-binding protein, s_pp015018333r-protein, sec-independent protein translocase, Sec-independent protein translocase subunit, serine acetyltransferase, serine hydroxymethyltransferase, serine protease, serine/threonine dehydratase, Serine/threonine-protein phosphatase, short-chain alcohol dehydrogenase family, sll0281-protein, sll1032-protein, sll1761-protein, sn-glycerol-3-phosphate dehydrogenase, sodium/proton antiporter, sterol 24-C-methyltransferase, thioredoxin, thioredoxin family protein, threonine dehydratase, threonine synthase, transcription factor, transcriptional regulator, transport protein, trehalose-phosphatase, tryptophan biosynthesis protein, tryptophan synthase alpha chain, TTC0768-protein, TTC0881-protein, TTC1386-protein, urease subunit, uridine/cytidine kinase, uroporphyrin-III C-methyltransferase, XM_{—}473199-protein, ydr183w-protein, ydr273w-protein, ydr507c-protein, yer014w-protein, yer106w-protein, YFL019C-protein, yfl054c-protein, ygl096w-protein, ygl237c-protein, ygr221c-protein, yhl013c-protein, yhr006w-protein, yhr207c-protein, YKL038W-protein, yml083c-protein, ymr013c-protein, YNL086W-protein, yol160w-protein, zinc finger protein, zinc transporter, and Zm_{—}4842_BE510522-protein are also called “FCRP genes”.

[0211.1.1.3] to [0225.1.1.3] for the disclosure of these paragraphs see [0211.1.1.1] to [0225.1.1.1] above.

In addition to the sequence mentioned in Table I, application no. 3, column 5 or 8, preferably the coding region thereof, or its homologs or fragments, it may be advantageous additionally to express and/or mutate further genes in the non-human organisms. Especially advantageously, additionally at least one further gene of the biosynthetic pathway of the fine chemical is expressed in the non-human organisms such as plants or microorganisms. It is also possible that the regulation of the natural genes has been modified advantageously so that the gene and/or its gene product is no longer subject to the regulatory mechanisms which exist in the non-human organisms. This leads to an increased synthesis of the respective amino acid since, for example, feedback regulations no longer exist to the same extent or not at all. In addition it might be advantageously to combine the nucleic acids sequences of the invention containing the sequences shown in the respective line in Table I, application no. 3, column 5 or 8, preferably the coding region thereof, or homologs or fragments thereof, with genes which generally support or enhance the growth or yield of the target non-human organisms, for example genes which lead to faster growth rate of non human organism like microorganisms or plants or genes which produce stress-, pathogen-, or herbicide-resistant plants.

[0227.1.1.3] to [0239.1.1.3] for the disclosure of these paragraphs see [0227.1.1.1] to [0239.1.1.1] above.

In an embodiment of the present invention the nucleic acid construct of the present invention comprises a nucleic acid molecule as depicted in the respective line in Table I, application no. 3, column 5 or 8, preferably the coding region thereof, a fragment or a homolog thereof, and a plant promoter, such as the Big 35S promoter, the DC3 promoter, the PC Ubi promoter, the LegB4-promoter, the Super promoter, the USP promoter or the like. In another embodiment the nucleic acid construct of the present invention comprises a nucleic acid molecule as depicted in the respective line in Table I, application no. 3, column 5 or 8, preferably the coding region thereof, a fragment or a homolog thereof, and a plant promoter such as, for example, the USP, the LegB4-, the DC3 promoter or the ubiquitin promoter from parsley or other herein mentioned promoters. In a further embodiment the nucleic acid construct of the present invention comprises a nucleic acid molecule as depicted in the respective line in Table I, application no. 3, column 5 or 8, preferably the coding region thereof, a fragment or a homolog thereof, and a plant promoter, such as the Big 35S promoter, the PC Ubi promoter, the Super promoter, the USP promoter or the like. In addition different terminators may advantageously be used in these nucleic acid constructs; examples for transcriptional termination are polyadenylation signals.

[241.1.1.3] to [0245.1.1.3] for the disclosure of these paragraphs see [0241.1.1.1] to [0245.1.1.1] above.

Other preferred sequences for use in operable linkage in gene expression constructs are targeting sequences, which are required for targeting the gene product into specific cell compartments (for a review, see Kermode, Crit. Rev. Plant Sci. 15 (4), 285 (1996) and references cited therein), for example into the vacuole, the nucleus, all types of plastids, such as amyloplasts, chloroplasts, chromoplasts, the extracellular space, the mitochondria, the endoplasmic reticulum, elaioplasts, peroxisomes, glycosomes, and other compartments of cells or extracellular; preferred are sequences, which are involved in targeting to plastids as mentioned above. Sequences, which must be mentioned in this context are, in particular, the signal-peptide- or transit-peptide-encoding sequences which are known per se. For example, plastid-transit-peptide-encoding sequences enable the targeting of the expression product into the plastids of a plant cell. Targeting sequences are also known for eukaryotic and to a lower extent for prokaryotic organisms and can advantageously be operable linked with the nucleic acid molecule of the present invention as shown in Table I, application no. 3, columns 5 and 8, preferably the coding region thereof, or homologs or fragments thereof, and described herein to achieve an expression in one of said compartments or extracellular.

[0247.1.1.3] to [0266.1.1.3] for the disclosure of these paragraphs see [0247.1.1.1] to [0266.1.1.1] above.

An embodiment of the invention relates to a vector where the nucleic acid molecule according to the invention is linked operably to regulatory sequences which permit the expression in a prokaryotic or eukaryotic host. A further embodiment of the invention relates to a vector in which a nucleic acid sequence encoding one of the polypeptides shown in the respective line in Table II, application no. 3, columns 5 or 8, or homologs or fragments thereof, is functionally linked to a targeting sequence such as a plastidial or mitochondrial targeting sequence. A further embodiment of the invention relates to a vector in which a nucleic acid sequence encoding one of the polypeptides shown in the respective line in Table II, application no. 3, columns 5 or 8, or homologs or fragments thereof, is functionally linked to (a) regulatory sequence which permit the expression in an organelle, such as plastids or mitochondria, preferably plastids.

A further embodiment of the invention relates to a vector which comprises a nucleic acid sequence encoding one of the polypeptides shown in the respective line in Table II, application no. 3, columns 5 or 8, or homologs or fragments thereof, but no additional targeting sequence.

[0268.1.1.3] to [0273.1.1.3] for the disclosure of these paragraphs see [0268.1.1.1] to [0273.1.1.1] above.

A further object of the invention relates to the use of a nucleic acid construct, e.g. an expression cassette, comprising one or more nucleic acid molecules encoding one or more polypeptides as depicted in the respective line(s) in Table II, application no. 3, columns 5 or 8, or homologs or fragments thereof, or comprising one or more nucleic acid molecules as depicted in the respective line(s) in Table I, application no. 3, columns 5 or 8, preferably the coding region thereof, or homologs or fragments thereof, or comprising a nucleic acid molecule hybridizing therewith for the transformation of a microorganism or a plant cell, plant tissues or a part of a plant

In doing so, depending on the choice of promoter, the nucleic acid molecules shown in the respective line in Table I, application no. 3, column 5 or 8, preferably the coding region thereof, or homologs or fragments thereof, or the nucleic acid molecules encoding a polypeptide as depicted in the respective line in Table II, application no. 3, column 5 or 8, or homologs or fragments thereof, can be expressed in the whole plant or specifically e.g. in the leaves, in the seeds, the nodules, in roots, in the stem or other parts of the plant. Those transgenic plants overproducing sequences, e.g. as depicted in the respective line in Table I, application no. 3, column 5 or 8, preferably the coding region thereof, or homologs or fragments thereof, the plant cell, plant tissues, plants, the reproductive material thereof, or parts thereof are a further object of the present invention.

Within the framework of the present invention, the production of the 3,4-dihydroxyphenylalanine (dopa), 5-oxoproline, alanine, asparagine, aspartate, citrulline, glycine, homoserine, ornithine, phenylalanine, serine, or tyrosine is due to the generation or overexpression of one or more polypeptides as depicted in the respective line(s) in Table II, application no. 3, column 5 or 8, or homologs or fragments thereof, or encoded by the corresponding nucleic acid molecules as depicted in the respective line(s) in Table I, application no. 3, column 5 or 8, preferably the coding region thereof, or homologs or fragments thereof, in the non-human organism according to the invention, advantageously in the transgenic microorganism or plant according to the invention, at least for the duration of at least one plant generation.

A constitutive expression of the polypeptide as depicted in the respective line in Table II, application no. 3, column 5 or 8, or homologs or fragments thereof, or encoded by the respective nucleic acid molecule as depicted in the respective line in Table I, application no. 3, column 5 or 8, preferably the coding region thereof, or homologs or fragments thereof, may be advantageous. On the other hand, an inducible expression may also appear desirable. Expression of the polypeptide sequences of the invention can be either directed to the cytosol or to the organelles, such as plastids or mitochondria, preferably the plastids of the host cells, preferably the plant cells, or non-targeted.

for the disclosure of this paragraph see [0278.1.1.1] above.

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 3, column 5 or 8, preferably shown in Table II A, application no. 3, column 5, or in Table II A, application no. 3, column 8, or in Table II B, application no. 3, column 8, or a homolog or a fragment thereof;
   (b) a nucleic acid molecule shown in Table I, application no. 3, column 5 or 8, preferably shown in Table I A, application no. 3, column 5, or in Table I A, application no. 3, column 8, or in Table I B, application no. 3, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 3, preferably shown in Table II A, application no. 3, column 5, or in Table II A, application no. 3, column 8, or in Table II B, application no. 3, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 3, preferably shown in Table I A, application no. 3, column 5, or in Table I A, application no. 3, column 8, or in Table I B, application no. 3, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 3;
   (i) a nucleic acid molecule encoding a polypeptide having the activity represented by a respective protein as depicted in column 5 of Table II, application no. 3;
   (j) a nucleic acid molecule comprising a polynucleotide which is obtained by amplifying a cDNA library or a genomic library using the primers in Table III, application no. 3, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
      or a nucleic acid molecule comprising a sequence which is complementary thereto; and, if desired
2) nucleic acid molecule encoding a transit peptide, preferably encoding a plastidal transit peptide or a mitochondrial transit peptide;
   which are operable linked;]

or comprising an expression cassette comprising [0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 3, column 5 or 8, preferably shown in Table II A, application no. 3, column 5, or in Table II A, application no. 3, column 8, or in Table II B, application no. 3, column 8, or a homolog or a fragment thereof;
   (b) a nucleic acid molecule shown in Table I, application no. 3, column 5 or 8, preferably shown in Table I A, application no. 3, column 5, or in Table I A, application no. 3, column 8, or in Table I B, application no. 3, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 3, preferably shown in Table II A, application no. 3, column 5, or in Table II A, application no. 3, column 8, or in Table II B, application no. 3, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 3, preferably shown in Table I A, application no. 3, column 5, or in Table I A, application no. 3, column 8, or in Table I B, application no. 3, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 3;
   (i) a nucleic acid molecule encoding a polypeptide having the activity represented by a respective protein as depicted in column 5 of Table II, application no. 3;
   (j) a nucleic acid molecule comprising a polynucleotide which is obtained by amplifying a cDNA library or a genomic library using the primers in Table III, application no. 3, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
      or a nucleic acid molecule comprising a sequence which is complementary thereto;
      and, if desired
2) nucleic acid molecule encoding a transit peptide, preferably encoding a plastidal transit peptide or a mitochondrial transit peptide; which are operable linked;]
   or comprising or being transformed by a vector comprising
[1) a nucleic acid molecule selected from the group consisting of
   (a) a nucleic acid molecule encoding a polypeptide shown in Table II, application no. 3, column 5 or 8, preferably shown in Table II A, application no. 3, column 5, or in Table II A, application no. 3, column 8, or in Table II B, application no. 3, column 8, or a homolog or a fragment thereof;
   (b) a nucleic acid molecule shown in Table I, application no. 3, column 5 or 8, preferably shown in Table I A, application no. 3, column 5, or in Table I A, application no. 3, column 8, or in Table I B, application no. 3, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 3, preferably shown in Table II A, application no. 3, column 5, or in Table II A, application no. 3, column 8, or in Table II B, application no. 3, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 3, preferably shown in Table I A, application no. 3, column 5, or in Table I A, application no. 3, column 8, or in Table I B, application no. 3, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 3;
   (i) a nucleic acid molecule encoding a polypeptide having the activity represented by a respective protein as depicted in column 5 of Table II, application no. 3;
   (j) a nucleic acid molecule comprising a polynucleotide which is obtained by amplifying a cDNA library or a genomic library using the primers in Table III, application no. 3, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
      or a nucleic acid molecule comprising a sequence which is complementary thereto;]
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 3.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 3 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 3, in the same line as of the respective nucleic acid molecule according to 1) “plastidic” is depicted, or encoding a mitochondrial transit peptide, especially in case in column 6 of Table I, application no. 3, in the same line as of the respective nucleic acid molecule according to 1) “mitochondrial” is depicted.

In another embodiment of said transgenic non-human organism the expression cassette does not comprise 2) a nucleic acid molecule encoding a transit peptide, especially in case in column 6 of Table I, application no. 3, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 3.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 3, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 3, in the same line as of the respective nucleic acid molecule according to 1) “plastidic” is depicted, or encoding a mitochondrial transit peptide, especially in case in column 6 of Table I, application no. 3, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 3, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 3, in the same line as of the respective nucleic acid molecule according to 1) “plastidic” is depicted, or encoding a mitochondrial transit peptide, especially in case in column 6 of Table I, application no. 3, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 3, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 3, in the same line as of the respective nucleic acid molecule according to 1) “plastidic” is depicted, or encoding a mitochondrial transit peptide, especially in case in column 6 of Table I, application no. 3, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 3.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 3, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 3.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 3, in the same line as of the respective nucleic acid molecule according to 1) “plastidic” is depicted, or encoding a mitochondrial transit peptide, especially in case in column 6 of Table I, application no. 3, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 3, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 3, in the same line as of the respective nucleic acid molecule according to 1) “plastidic” is depicted, or encoding a mitochondrial transit peptide, especially in case in column 6 of Table I, application no. 3, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 3 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 3, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 3 (in the same line) and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 3, column 5 or 8, preferably shown in Table II A, application no. 3, column 5, or in Table II A, application no. 3, column 8, or in Table II B, application no. 3, column 8, or a homolog or a fragment thereof;
   (b) a nucleic acid molecule shown in Table I, application no. 3, column 5 or 8, preferably shown in Table I A, application no. 3, column 5, or in Table I A, application no. 3, column 8, or in Table I B, application no. 3, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 3, preferably shown in Table II A, application no. 3, column 5, or in Table II A, application no. 3, column 8, or in Table II B, application no. 3, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 3, preferably shown in Table I A, application no. 3, column 5, or in Table I A, application no. 3, column 8, or in Table I B, application no. 3, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 3;
   (i) a nucleic acid molecule encoding a polypeptide having the activity represented by a respective protein as depicted in column 5 of Table II, application no. 3;
   (j) a nucleic acid molecule comprising a polynucleotide which is obtained by amplifying a cDNA library or a genomic library using the primers in Table III, application no. 3, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
      or a nucleic acid molecule comprising a sequence which is complementary thereto;
      and, if desired
2) nucleic acid molecule encoding a transit peptide, preferably encoding a plastidal transit peptide or a mitochondrial transit peptide; which are operable linked;]
   or comprising an expression cassette comprising
[0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 3, column 5 or 8, preferably shown in Table II A, application no. 3, column 5, or in Table II A, application no. 3, column 8, or in Table II B, application no. 3, column 8, or a homolog or a fragment thereof;
   (b) a nucleic acid molecule shown in Table I, application no. 3, column 5 or 8, preferably shown in Table I A, application no. 3, column 5, or in Table I A, application no. 3, column 8, or in Table I B, application no. 3, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 3, preferably shown in Table II A, application no. 3, column 5, or in Table II A, application no. 3, column 8, or in Table II B, application no. 3, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 3, preferably shown in Table I A, application no. 3, column 5, or in Table I A, application no. 3, column 8, or in Table I B, application no. 3, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 3;
   (i) a nucleic acid molecule encoding a polypeptide having the activity represented by a respective protein as depicted in column 5 of Table II, application no. 3;
   (j) a nucleic acid molecule comprising a polynucleotide which is obtained by amplifying a cDNA library or a genomic library using the primers in Table III, application no. 3, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
      or a nucleic acid molecule comprising a sequence which is complementary thereto;]
      and, if desired
2) nucleic acid molecule encoding a transit peptide, preferably encoding a plastidal transit peptide or a mitochondrial transit peptide;
   which are operable linked;]
   or comprising or being transformed by a vector comprising
[1) a nucleic acid molecule selected from the group consisting of
   (a) a nucleic acid molecule encoding a polypeptide shown in Table II, application no. 3, column 5 or 8, preferably shown in Table II A, application no. 3, column 5, or in Table II A, application no. 3, column 8, or in Table II B, application no. 3, column 8, or a homolog or a fragment thereof;
   (b) a nucleic acid molecule shown in Table I, application no. 3, column 5 or 8, preferably shown in Table I A, application no. 3, column 5, or in Table I A, application no. 3, column 8, or in Table I B, application no. 3, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 3, preferably shown in Table II A, application no. 3, column 5, or in Table II A, application no. 3, column 8, or in Table II B, application no. 3, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 3, preferably shown in Table I A, application no. 3, column 5, or in Table I A, application no. 3, column 8, or in Table I B, application no. 3, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 3;
   (i) a nucleic acid molecule encoding a polypeptide having the activity represented by a respective protein as depicted in column 5 of Table II, application no. 3;
   (j) a nucleic acid molecule comprising a polynucleotide which is obtained by amplifying a cDNA library or a genomic library using the primers in Table III, application no. 3, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
      or a nucleic acid molecule comprising a sequence which is complementary thereto;]
      whereby, preferably, the nucleic acid molecule according to 1 (a), (b), (c), (d), (e), (f), (g), (h), (i), (j) or (k) distinguishes over the sequence depicted in Table I A and/or I B, application no. 3, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 3, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 3, column 5 or 8, or the coding region thereof, respectively. In a further embodiment the nucleic acid molecule does not encode the polypeptide sequence shown in Table II A and/or II B, application no. 3, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 3, column 5 or 8 and does not encode a protein of the sequence shown in Table II A and/or II B, application no. 3, column 5 or 8. Accordingly, in one embodiment, the protein encoded by a sequence of a nucleic acid according to 1 (a), (b), (c), (d), (e), (f), (g), (h), (i), (j) or (k) does not consist of the sequence shown in Table I A and/or I B, application no. 3, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 3, column 5 or 8 but less than 100%, preferably less than 99.999%, 99.99%, 99.9%, 99%, 98%, 97%, 96% or 95% identical to the sequence shown in Table II A and/or II B, application no. 3, column 5 or 8;
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 3.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 3 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 3, in the same line as of the respective nucleic acid molecule according to 1) “plastidic” is depicted, or encoding a mitochondrial transit peptide, especially in case in column 6 of Table I, application no. 3, in the same line as of the respective nucleic acid molecule according to 1) “mitochondrial” is depicted.

In another embodiment thereof the expression cassette does not comprise 2) a nucleic acid molecule encoding a transit peptide, especially in case in column 6 of Table I, application no. 3, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 3.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the same line in column 5 of Table II, application no. 3, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 3, in the same line as of the respective nucleic acid molecule according to 1) “plastidic” is depicted, or encoding a mitochondrial transit peptide, especially in case in column 6 of Table I, application no. 3, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 3, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 3, in the same line as of the respective nucleic acid molecule according to 1) “plastidic” is depicted, or encoding a mitochondrial transit peptide, especially in case in column 6 of Table I, application no. 3, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 3, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 3, in the same line as of the respective nucleic acid molecule according to 1) “plastidic” is depicted, or encoding a mitochondrial transit peptide, especially in case in column 6 of Table I, application no. 3, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 3.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 3, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 3.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 3, in the same line as of the respective nucleic acid molecule according to 1) “plastidic” is depicted, or encoding a mitochondrial transit peptide, especially in case in column 6 of Table I, application no. 3, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 3, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 3, in the same line as of the respective nucleic acid molecule according to 1) “plastidic” is depicted, or encoding a mitochondrial transit peptide, especially in case in column 6 of Table I, application no. 3, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 3 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 3, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 3 and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

[0291.1.1.3] to [0299.1.1.3] for the disclosure of these paragraphs see [0291.1.1.1] to [0299.1.1.1] above.

The term “biological active portion” or “biological activity” means a polypeptide as depicted in the respective line in Table II, application no. 3, column 3 or a portion of said polypeptide which still has at least 10% or 20%, preferably 30%, 40%, 50% or 60%, especially preferably 70%, 75%, 80%, 90% or 95% of the enzymatic or biological activity of the natural or starting enzyme or protein.

[0301.1.1.3] to [0304.1.1.3] for the disclosure of these paragraphs see [0301.1.1.1] to [0304.1.1.1 above.

Synthetic oligonucleotide primers for the amplification, e.g. as shown in the respective line in Table III, application no. 3, column 8, by means of polymerase chain reaction can be generated on the basis of a sequence shown herein, for example the sequence shown in the respective line in Table I, application no. 3, columns 5 or 8, or the sequences derived from Table II, application no. 3, columns 5 or 8, respectively.

Moreover, it is possible to identify a conserved region of a protein from various organism by carrying out protein sequence alignments with the polypeptide encoded by the nucleic acid molecules of the present invention, in particular with the sequences encoded by the nucleic acid molecule shown in the respective line in column 5 or 8 of Table I, application no. 3, from which conserved regions, and in turn, degenerate primers can be derived. Conserved regions are those, which show a very little variation in the amino acid in one particular position of several homologs from different origin. The consensus sequence and polypeptide motifs shown in the respective line in column 8 of Table IV, application no. 3, are derived from such alignments of homologous proteins.

In an embodiment of the present invention, in the method of the present invention the activity of a polypeptide comprising or consisting of a consensus sequence or at least one polypeptide motif shown in the respective line in Table IV, application no. 3, column 8, is increased. In another embodiment thereof, the method of the present invention relates to a polypeptide comprising or consisting of a consensus sequence shown in the respective line in Table IV, application no. 3, column 8 or comprising at least one polypeptide motif shown in the respective line in Table IV, application no. 3, column 8, whereby not more than 20, 15, 10, 9, 8, 7, 6, 5 4, 3, 2 or 1, or 0 of the amino acid positions indicated can be replaced by any amino acid. In another embodiment thereof, the method of the present invention relates to a polypeptide comprising or consisting of a consensus sequence shown in the respective line in Table IV, application no. 3, column 8 or comprising at least one polypeptide motif shown in the respective line in Table IV, application no. 3, column 8, whereby not more than 15%, 10%, 5%, 4%, 3%, 2% or 1%, or 0% of the amino acid positions defined as distinct specific amino acids are/is replaced by another amino acid. In another embodiment thereof, the method of the present invention relates to a polypeptide comprising or consisting of a consensus sequence shown in the respective line in Table IV, application no. 3, column 8 or comprising at least one polypeptide motif shown in the respective line in Table IV, application no. 3, column 8, whereby not more than 20, 15, 10, 9, 8, 7, 6, 5, 4, 3, 2 or 1, or 0 amino acids are inserted into a consensus sequence or polypeptide motif.

In an embodiment of the present invention, in the method of the present invention the activity of a polypeptide comprising or consisting of a consensus sequence or at least one polypeptide motif shown in the respective line in Table IV, application no. 3, column 8, is increased, whereby said polypeptide distinguishes over the sequence depicted in Table II, application no. 3, columns 5 or 8 by one or more amino acids.

In one embodiment, said polypeptide of the invention distinguishes over the sequence shown in the respective line in Table II, application no. 3, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II B, application no. 3, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in Table II, application no. 3, columns 5 or 8.

[0309.1.1.3] to [0321.1.1.3] for the disclosure of these paragraphs see [0309.1.1.1] to [0321.1.1.1] above.

Polypeptides having above-mentioned activity, i.e. conferring the production or the increased production of the fine chemical 3,4-dihydroxyphenylalanine (dopa), 5-oxoproline, alanine, asparagine, aspartate, citrulline, glycine, homoserine, ornithine, phenylalanine, serine, or tyrosine as compared to a corresponding, e.g. non-transformed, wild type non-human organism, like a microorganism or a plant cell, plant or part thereof, can be encoded by other DNA sequences which hybridize to the sequences shown in the respective line in Table I, application no. 3, columns 5 and 8, preferably the coding region thereof, at least under relaxed hybridization conditions and which encode the expression of polypeptides conferring the production or the increased production of the respective fine chemical 3,4-dihydroxyphenylalanine (dopa), 5-oxoproline, alanine, asparagine, aspartate, citrulline, glycine, homoserine, ornithine, phenylalanine, serine, or tyrosine as compared to a corresponding, e.g. non-transformed, wild type non-human organism, like a microorganism or a plant cell, plant or part thereof.

[0323.1.1.3] to [0329.1.1.3] for the disclosure of these paragraphs see [0323.1.1.1] to [0329.1.1.1] above.

Further, the nucleic acid molecule of the invention comprises a nucleic acid molecule, which is a complement of one of the nucleotide sequences of above-mentioned nucleic acid molecules or a portion thereof. A nucleic acid molecule or its sequence which is complementary to one of the nucleotide molecules or sequences shown in the respective line in Table I, application no. 3, columns 5 or 8, preferably the coding region thereof, homologs or fragments thereof, is one which is sufficiently complementary to one of the nucleotide molecules or sequences shown in the respective line in Table I, application no. 3, columns 5 or 8, preferably the coding region thereof, homologs or fragments thereof, such that it can hybridize to one of the nucleotide sequences shown in Table I, application no. 3, columns 5 or 8, preferably the coding region thereof, homologs or fragments thereof, thereby forming a stable duplex. Preferably, the hybridization is performed under stringent hybridization conditions. However, a complement of one of the herein disclosed sequences is preferably a sequence complement thereto according to the base pairing of nucleic acid molecules well known to the skilled person. For example, the bases A and G undergo base pairing with the bases T and U or C, resp. and visa versa. Modifications of the bases can influence the base-pairing partner.

The nucleic acid molecule of the invention comprises a nucleotide sequence which is at least about 30%, 35%, 40%, 45%, 50%, 55%, 60%, 65%, 70%, 75%, 80%, 90%, 95%, 96%, 97%, 98%, 99%, 99.5% or more homologous to a nucleotide sequence shown in Table I, application no. 3, columns 5 or 8, preferably the coding region thereof, homologs or fragments thereof, and preferably has above-mentioned activity, in particular conferring the production or the increased production of the fine chemical 3,4-dihydroxyphenylalanine (dopa), 5-oxoproline, alanine, asparagine, aspartate, citrulline, glycine, homoserine, ornithine, phenylalanine, serine, or tyrosine, respectively, after increasing the activity or an activity of a gene as shown in the respective line in Table I or of a gene product, e.g. as shown in the respective line in Table II, application no. 3, column 5 or 8, by for example in one embodiment expression either in the cytosol or in an organelle such as a plastid or mitochondria or both, preferably in a plastid, or in another embodiment non-targeted or targeted.

The nucleic acid molecule of the invention comprises a nucleotide sequence or molecule which hybridizes, preferably hybridizes under stringent conditions as defined herein, to one of the nucleic acid molecule shown in the respective line in Table I, preferably Table IB, application no. 3, columns 5 or 8, preferably the coding region thereof, fragments or homologs thereof, and encodes a protein having above-mentioned activity, e.g. conferring the production or the increased production of the respective fine chemical 3,4-dihydroxyphenylalanine (dopa), 5-oxoproline, alanine, asparagine, aspartate, citrulline, glycine, homoserine, ornithine, phenylalanine, serine, or tyrosine as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted, and optionally, the activity thereof is selected from the group consisting of 2,3-dihydroxyphenylpropionate 1,2-dioxygenase, 2-C-methyl-D-erythritol 4-phosphate cytidylyltransferase, 2-oxoglutarate dehydrogenase E1 subunit, 30S ribosomal protein S3, 3-deoxy-7-phosphoheptulonate synthase, 3-deoxy-D-arabinoheptulosonate-7-phosphatesynthase, 3-oxoacyl-[acyl-carrier-protein] synthase, 3-phosphoglycerate dehydrogenase, 47266012-protein, 49747384_SOYBEAN-protein, 50S ribosomal protein L14, 5′nucleotidase, ABC metal ion transporter substrate-binding protein, ABC transporter ATP binding component, ABC transporter ATP-binding protein, ABC transporter component, ABC transporter permease protein, ABC transporter substrate-binding protein, acetolactate synthase, acetolactate synthase small subunit, acetyl CoA carboxylase, acetyl-CoA acetyltransferase, acetyltransferase, acid shock protein, acyl transferase, acyl-CoA dehydrogenase, acyl-CoA synthase, adenylate kinase, adenylylsulfate kinase, agmatinase, aldehyde dehydrogenase, amine oxidase, amino acid ABC transporter permease protein, amino acid acetyltransferase, amino acid transporter, amino-acid acetyltransferase, aminotransferase, ammonium transporter, ankyrin repeat family protein, anthranilate synthase component I, arginine exporter protein, arginine N-succinyltransferase, argininosuccinate lyase, asparaginase, aspartate 1-decarboxylase, aspartate aminotransferase, aspartate kinase, aspartate-ammonia ligase, At1g17440-protein, At1g19800-protein, At4g32480-protein, At5g16650-protein, at5g21910-protein, ATP-binding component of a transport system, ATP-dependent RNA helicase, auxin response factor, AX653549-protein, AY087308-protein, b0391-protein, b0456-protein, b0518-protein, b0801-protein, b0841-protein, b0917-protein, b1003-protein, b1024-protein, b1137-protein, b1155-protein, b1163-protein, b1179-protein, b1205-protein, b1229-protein, b1259-protein, b1280-protein, b1330-protein, b1445-protein, b1522-protein, b1583-protein, b1670-protein, b1672-protein, b1837-protein, b1898-protein, b1930-protein, b2032-protein, b2107-protein, b2360-protein, b2399-protein, b2474-protein, b2513-protein, b2613-protein, b2673-protein, b2739-protein, b2812-protein, b2849-protein, b3083-protein, b3098-protein, b3121-protein, b3221-protein, b3246-protein, b3346-protein, b3392-protein, b3410-protein, b3509-protein, b3646-protein, b3791-protein, b3814-protein, b3989-protein, b4029-protein, b4121-protein, beta-galactosidase, beta-hydroxylase, bifunctional aspartokinase/homoserine dehydrogenase, bifunctional purine biosynthesis protein, branched chain aminotransferase, branched-chain amino acid permease, calcium-dependent protein kinase, carbon dioxide concentrating mechanism protein, cation-transporting ATPase, CBL-interacting protein kinase, CDP-diacylglycerol-glycerol-3-phosphate 3-phosphatidyltransferase, CDP-diacylglycerol-serine O-phosphatidyltransferase, CDP-glucose-4,6-dehydratase, cell division control protein, cellulose synthase catalytic subunit, Chaperone protein CIpB, chorismate mutase-T and prephenate dehydrogenase, citrate synthase, coproporphyrinogen III oxidase, CTP synthetase, cullin, cyclin D, cystathionine gamma-synthase, cytochrome c-type protein, D-3-phosphoglycerate dehydrogenase, delta-1-pyrroline 5-carboxylase synthetase, diacylglycerol O-acyltransferase, dihydroxyacid dehydratase, DNA helicase II, DNA mismatch repair protein, DNA-binding protein, DnaJ-like chaperone, D-serine dehydratase, electron transfer flavoprotein subunit beta, elongation factor Tu, enoyl-CoA hydratase, enterobacterial common antigen polymerase, eukaryotic translation initiation factor, eukaryotic translation initiation factor 5, exopolyphosphatase, Fe—S subunit of oxidoreductase, flavodoxin, fumarylacetoacetate hydrolase, gamma-glutamyltranspeptidase, GDP-mannose dehydrogenase, geranylgeranyl pyrophosphate synthase, gibberellin 20-oxidase, Glu/Leu/Phe/Val dehydrogenase , glucokinase, glucose dehydrogenase, glucose-1-phosphate cytidylyltransferase, glucose-6-phosphate 1-dehydrogenase, glutamate-ammonia-ligase, glutamine amidotransferase, glutamine synthetase, glutaredoxin, glutathione peroxidase, glutathione S-transferase, Glycine cleavage system T aminomethyltransferase, glycogen (starch) synthase, glycogen synthase, glycoprotease, glycosyl transferase, GM02LC11114protein, GM02LC17485-protein, harpin-induced family protein, heat shock transcription factor, HesB/YadR/YfhF family protein, histone acetyltransferase, histone H2A, homocitrate synthase, homoserine O-acetyltransferase, hydrolase, integral membrane transporter family protein, iron(III) dicitrate-binding protein, isochorismate synthase, kinase, L-aspartate oxidase, lipoprotein precursor, lysine decarboxylase, lysyl-tRNA synthetase, major facilitator superfamily transporter protein, malate dehydrogenase, malic enzyme, membrane protein, membrane transport protein, metal ion transporter, metal-dependent hydrolase, methylglyoxal synthase, methylglyoxalase, methyltransferase, mitochondrial inner membrane protease subunit, molecular chaperone portein, monothiol glutaredoxin, monthiol glutaredoxin, multidrug resistance protein, multiple antibiotic resistance protein, murein transglycosylase, N-(5′-phosphoribosyl)anthranilate isomerase, Na+/K+ transporter, N-acetyl-gamma-glutamyl-phosphate reductase, NADH dehydrogenase I chain I, NADH dehydrogenase subunit N, NADH-quinone oxidoreductase subunit, NADP-dependent malic enzyme, nitrate/nitrite transport protein, O-antigen chain length determinant, oxidoreductase, oxidoreductase subunit, oxireductase, peptidase, permease protein of phosphate ABC transporter, phosphate permease, phosphate transporter, phosphoadenosine phosphosulfate reductase , phosphoanhydride phosphorylase, phosphoenolpyruvate carboxykinase, phosphopantetheine adenylyltransferase, phosphoribosylformyl glycinamidine synthase subunit, Photosystem I reaction center subunit XI, photosystem II protein, polygalacturonase, precorrin methylase, Pre-mRNA-splicing factor, prolipoprotein diacylglyceryl transferase, protease, protein kinase, protein phosphatase, PTS system N-acetylmuramic acid-specific EIIBC component, purine biosynthesis protein, pyruvate kinase, restriction alleviation protein, riboflavin biosynthesis protein, RNA binding protein, RNA polymerase sigma factor, RNA-binding protein, s_pp015018333r-protein, sec-independent protein translocase, Sec-independent protein translocase subunit, serine acetyltransferase, serine hydroxymethyltransferase, serine protease, serine/threonine dehydratase, Serine/threonine-protein phosphatase, short-chain alcohol dehydrogenase family, sll0281-protein, sll1032-protein, sll1761-protein, sn-glycerol-3-phosphate dehydrogenase, sodium/proton antiporter, sterol 24-C-methyltransferase, thioredoxin, thioredoxin family protein, threonine dehydratase, threonine synthase, transcription factor, transcriptional regulator, transport protein, trehalose-phosphatase, tryptophan biosynthesis protein, tryptophan synthase alpha chain, TTC0768-protein, TTC0881-protein, TTC1386protein, urease subunit, uridine/cytidine kinase, uroporphyrin-III C-methyltransferase, XM_{—}473199-protein, ydr183w-protein, ydr273w-protein, ydr507c-protein, yer014w-protein, yer106w-protein, YFL019C-protein, yfl054c-protein, ygl096w-protein, ygl237c-protein, ygr221 c-protein, yhl013c-protein, yhr006w-protein, yhr207c-protein, YKL038W-protein, yml083c-protein, ymr013c-protein, YNL086W-protein, yol160w-protein, zinc finger protein, zinc transporter, and Zm_{—}4842_BE510522-protein, respectively.

Moreover, the nucleic acid molecule of the invention can comprise only a portion of the coding region of one of the nucleic acid molecules depicted in Table I, application no. 3, columns 5 or 8, for example a fragment which can be used as a probe or primer or a fragment encoding a biologically active portion of the polypeptide of the present invention or of a polypeptide used in the process of the present invention, i.e. having above-mentioned activity, e.g. conferring the production or the increased production of the respective fine chemical 3,4dihydroxyphenylalanine (dopa), 5-oxoproline, alanine, asparagine, aspartate, citrulline, glycine, homoserine, ornithine, phenylalanine, serine, or tyrosine as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted. The nucleotide sequences determined from the cloning of the present protein-according-to-the-invention-encoding gene allows for the generation of probes and primers designed for use in identifying and/or cloning its homologues from other cell types and organisms. The probe/primer typically comprises a substantially purified oligonucleotide. The oligonucleotide typically comprises a region of nucleotide sequence that hybridizes under stringent conditions to at least about 12, 15 preferably about 20 or 25, more preferably about 40, 50 or 75 consecutive nucleotides of a sense strand of one of the sequences set forth, e.g., in the respective line in Table I, application no. 3, column 5 or 8, preferably the coding region thereof, or a homolog or a fragment thereof, an anti-sense sequence of one of the sequences, e.g., set forth in the respective line in Table I, application no. 3, columns 5 or 8, preferably the coding region thereof, or a homolog or a fragment thereof or naturally occurring mutants thereof. Primers based on a nucleic acid molecule of invention can be used in PCR reactions to clone homologs of the polypeptide of the invention or of the polypeptide used in the process of the invention, e.g. as the primers described in the examples of the present invention, e.g. as shown in the examples. A PCR with the primers shown in the respective line in Table Ill, column 8 will result in a fragment of the gene product as shown in Table II, application no. 3, column 5.

Primer sets are interchangeable. The person skilled in the art knows how to design and combine said primers to result in the desired product, e.g. in a full length clone or a partial sequence. Probes based on the sequences of the nucleic acid molecule of the invention or used in the process of the present invention can be used to detect transcripts or genomic sequences encoding the same or homologous proteins. The probe can further comprise a label group attached thereto, e.g. the label group can be a radioisotope, a fluorescent compound, an enzyme, or an enzyme co-factor. Such probes can be used as a part of a genomic marker test kit for identifying cells which express a polypeptide of the invention or used in the process of the present invention, such as by measuring a level of an encoding nucleic acid molecule in a sample of cells, e.g. detecting mRNA levels or determining, whether a genomic gene comprising the sequence of the polynucleotide of the invention or used in the processes of the present invention has been mutated or deleted.

As the expression of a respective nucleic acids of the invention is related to the synthesis of the respective fine chemical 3,4-dihydroxyphenylalanine (dopa), 5-oxoproline, alanine, asparagine, aspartate, citrulline, glycine, homoserine, ornithine, phenylalanine, serine, or tyrosine its function as a probe extends to the detection of microorganisms, plant tissues, plants, plant variets, plant ecotypes or plant genera with varying capability or potential for synthesis of the respective fine chemical 3,4-dihydroxyphenylalanine (dopa), 5-oxoproline, alanine, asparagine, aspartate, citrulline, glycine, homoserine, ornithine, phenylalanine, serine, or tyrosine. Therefore in one embodiment the present invention relates to a method for analyzing the capability or potential of a plant tissue, a plant, a plant variety or plant ecotype to produce the fine chemical 3,4-dihydroxyphenylalanine (dopa), 5-oxoproline, alanine, asparagine, aspartate, citrulline, glycine, homoserine, ornithine, phenylalanine, serine, or tyrosine by using the nucleic acid of the invention or parts thereof as a probe to detect the amount of the nucleic acid of the invention in the non-human organism or a part thereof in comparison to another non-human organism.

The nucleic acid molecule of the invention encodes a polypeptide or portion thereof which includes an amino acid sequence which is sufficiently homologous to the amino acid sequence shown in respective line in Table II, application no. 3, columns 5 or 8 such that the protein or portion thereof maintains the ability to confer the production or the increased production of the respective fine chemical 3,4-dihydroxyphenylalanine (dopa), 5-oxoproline, alanine, asparagine, aspartate, citrulline, glycine, homoserine, ornithine, phenylalanine, serine, or tyrosine as compared to a corresponding, e.g. non-transformed, wild type non-human organism, like a microorganism or a plant cell, plant or part thereof, in particular increasing the activity as mentioned above or as described in the examples in microorganisms or plants.

As used herein, the language “sufficiently homologous” refers to proteins or portions thereof which have amino acid sequences which include a minimum number of identical or equivalent amino acids (e.g. an amino acid which has a similar side chain as an amino acid in one of the sequences of the polypeptide of the present invention) to an amino acid sequence shown in the respective line in Table II, application no. 3, columns 5 or 8 such that the protein or portion thereof is able to confer the production or the increased production of the respective fine chemical 3,4-dihydroxyphenylalanine (dopa), 5-oxoproline, alanine, asparagine, aspartate, citrulline, glycine, homoserine, ornithine, phenylalanine, serine, or tyrosine as compared to a corresponding, e.g. non-transformed, wild type non-human organism, like a microorganism or a plant cell, plant or part thereof. For example having the activity of a protein as shown in the respective line in Table II, application no. 3, column 3 and as described herein.

In an embodiment of the present invention, the nucleic acid molecule of the present invention comprises a nucleic acid molecule that encodes a portion of the protein of the present invention. The protein is at least about 30%, 35%, 40%, 45% 50%, 55%, 60%, 65%, 70%, 75%, 80%, 85%, 90%, 91%, 92%, 93%, 94%, 95%, 96%, 97%, 98%, 99%, 99.5% or more homologous to an entire amino acid sequence shown in the respective line of Table II, application no. 3, column 5 or 8 and has the above-mentioned activity, e.g. conferring the production or the increased production of the respective fine chemical 3,4-dihydroxyphenylalanine (dopa), 5-oxoproline, alanine, asparagine, aspartate, citrulline, glycine, homoserine, ornithine, phenylalanine, serine, or tyrosine as compared to a corresponding, e.g. non-transformed, wild type non-human organism, like a microorganism or a plant cell, plant or part thereof by, for example in a embodiment expression either in the cytosol or in an organelle such as a plastid or mitochondria or both, preferably in a plastid, or in another embodiment by targeted or non-targeted expression.

[0338.1.1.3] to [0339.1.1.3] for the disclosure of these paragraphs see [0338.1.1.1] to [0339.1.1.1] above.

The invention further relates to nucleic acid molecules or methods using said nucleic acid molecules that differ from one of the nucleotide sequences shown in the respective line in Table I, application no. 3, columns 5 or 8, preferably the coding region thereof, or fragments or homologs thereof, due to degeneracy of the genetic code but encode a polypeptide of the present invention, in particular a polypeptide having the above-mentioned activity, e.g. as those polypeptides depicted in the respective line in Table II, application no. 3, columns 5 or 8, fragments or homologs thereof. Advantageously, the nucleic acid molecule of the invention comprises, or in an other embodiment has, a nucleotide sequence encoding a protein comprising, or in an other embodiment having, an amino acid sequence shown in the respective line in Table II, application no. 3, columns 5 or 8, fragments or homologs thereof. In a further embodiment, the nucleic acid molecule of the invention encodes a full length protein which is substantially homologous to an amino acid sequence shown in the respective line in Table II, application no. 3, columns 5 or 8, or homologs thereof. However, in an embodiment, the nucleic acid molecule of the present invention does not consist of the sequence shown in the respective line in Table I, preferably Table IA, application no. 3, columns 5 or 8, preferably the coding region thereof, but the functional homologs thereof.

[0341.1.1.3] to [0343.1.1.3] for the disclosure of these paragraphs see [0341.1.1.1] to [0343.1.1.1] above.

Accordingly, in another embodiment, a nucleic acid molecule of the invention is at least 15, 20, 25 or 30 nucleotides in length. Preferably, it hybridizes under stringent conditions to a nucleic acid molecule comprising a nucleotide sequence of the nucleic acid molecule of the present invention or used in the process of the present invention, e.g. comprising the sequence shown in the respective line in Table I, application no. 3, columns 5 or 8, preferably the coding region thereof. In another embodiment the nucleic acid molecule is preferably at least 20, 30, 50, 100, 250 or more nucleotides in length.

for the disclosure of this paragraph see [0345.1.1.1] above.

Preferably, a nucleic acid molecule of the invention that hybridizes under stringent conditions to a sequence shown in the respective line in Table I, application no. 3, columns 5 or 8, preferably the coding region thereof, corresponds to a naturally-occurring nucleic acid molecule of the invention. As used herein, a “naturally-occurring” nucleic acid molecule refers to a RNA or DNA molecule having a nucleotide sequence that occurs in nature (e.g. encodes a natural protein). Preferably, the nucleic acid molecule encodes a natural protein having above-mentioned activity, e.g. conferring the production or the increased production of the respective fine chemical 3,4-dihydroxyphenylalanine (dopa), 5-oxoproline, alanine, asparagine, aspartate, citrulline, glycine, homoserine, ornithine, phenylalanine, serine, or tyrosine as compared to a corresponding, e.g. non-transformed, wild type non-human organism, like a microorganism or a plant cell, plant or part thereof after increasing the expression or activity thereof or the activity of a protein of the invention or used in the process of the invention, in an embodiment for example expression either in the cytosol or in an organelle such as a plastid or mitochondria, preferably in plastids, or, in another embodiment by targeted or non-targeted expression.

for the disclosure of this paragraph see [0347.1.1.1] above.

For example, nucleotide substitutions leading to amino acid substitutions at “non-essential” amino acid residues can be made in a sequence of the nucleic acid molecule of the invention or used in the process of the invention, e.g. shown in the respective line in Table I, application no. 3, columns 5 or 8, preferably the coding region thereof, or fragments or homologs thereof.

[0349.1.1.3] to [0350.1.1.3] for the disclosure of these paragraphs see [0349.1.1.1] to [0350.1.1.1] above.

Accordingly, the invention relates to nucleic acid molecules encoding a polypeptide having above-mentioned activity, in a non-human organisms or parts thereof by for example expression either in the cytosol or in an organelle such as a plastid or mitochondria or both, preferably in plastids, that contain changes in amino acid residues that are not essential for said activity. Such polypeptides differ in amino acid sequence from a sequence depicted in the respective line in Table II, especially Table IIA, application no. 3, columns 5 or 8, but retain said activity described herein. The nucleic acid molecule can comprise a nucleotide sequence encoding a polypeptide, wherein the polypeptide comprises an amino acid sequence at least about 50% identical to an amino acid sequence shown in the respective line in Table II, application no. 3, columns 5 or 8, and is capable of conferring the production or the increased production of the respective fine chemical 3,4-dihydroxyphenylalanine (dopa), 5-oxoproline, alanine, asparagine, aspartate, citrulline, glycine, homoserine, ornithine, phenylalanine, serine, or tyrosine as compared to a corresponding, e.g. non-transformed, wild type non-human organism, like a microorganism or a plant cell, plant or part thereof, after increasing its activity for example in an embodiment by expression either in the cytosol or in an organelle such as a plastid or mitochondria or both, preferably in plastids, or, in another embodiment by targeted or non-targeted expression. Preferably, the protein encoded by the nucleic acid molecule is at least about 60%, 70%, 80%, 90%, 95%, 96%, 97%, 98%, 99% or 99.5% identical to the sequence shown in the respective line in Table II, application no. 3, columns 5 or 8.

[0352.1.1.3] to [0357.1.1.3] for the disclosure of these paragraphs see [0352.1.1.1] to [0357.1.1.1] above.

Functional equivalents of the nucleic acid molecules according to the present invention and/or used in the process according to the present invention represent an embodiment of homologs. Functional equivalents derived from the nucleic acid molecules as shown in the respective line in Table I, application no. 3, columns 5 or 8, preferably the coding region thereof, according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the nucleic acid molecules as shown in the respective line in Table I, application no. 3, columns 5 or 8, preferably the coding region thereof, according to the invention and encode polypeptides having essentially the same properties as the polypeptide as shown in the respective line in Table II, application no. 3, columns 5 or 8.

Functional equivalents derived from one of the polypeptides as shown in the respective line in Table II, application no. 3, columns 5 or 8 according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the polypeptides as shown in the respective line in Table II, application no. 3, columns 5 or 8 according to the invention and having essentially the same properties as the polypeptide as shown in the respective line in Table II, application no. 3, columns 5 or 8.

for the disclosure of this paragraph see [0359.1.1.1] above.

A nucleic acid molecule encoding a homolog to a polypeptide depicted in the respective line in Table II, application no. 3, columns 5 or 8 can be created by introducing one or more nucleotide substitutions, additions or deletions into the nucleic acid molecule of the present invention, in particular of Table I, application no. 3, columns 5 or 8, in the respective line, such that one or more amino acid substitutions, additions or deletions are introduced into the encoded protein. Mutations can be introduced into the nucleic acid molecules as depicted in the respective line in Table I, application no. 3, columns 5 or 8, preferably the coding region thereof, by standard techniques, such as site-directed mutagenesis, PCR-mediated mutagenesis or other methods known to the person skilled in the art.

[0361.1.1.3] to [0363.1.1.3] for the disclosure of these paragraphs see [0361.1.1.1] to [0363.1.1.1] above.

Homologues of the nucleic acid sequences used, with the sequences shown in the respective line in Table I, application no. 3, columns 5 or 8, preferably the coding region thereof, comprise also allelic variants with at least approximately 30%, 35%, 40%, 45%, 50%, 55%, 60%, 65%, 70%, 75%, 80%, 85%, 90%, 91%, 92%, 93%, 94% 95%. 96%, 97%, 98%, 99%, 99.5% or more % homology with one of the nucleotide sequences shown or the above-mentioned derived nucleic acid sequences or their homologues or derivatives or parts of these. Allelic variants encompass in particular functional variants which can be obtained by deletion, insertion or substitution of nucleotides from the sequences shown in the respective line, preferably in Table I, columns 5 or 8, preferably the coding region thereof, or from the derived nucleic acid sequences, the intention being, however, that the enzymatic activity or the biological activity of the resulting proteins synthesized is advantageously retained or increased.

In one embodiment of the present invention, the nucleic acid molecule of the invention or used in the process of the invention comprises the sequences shown in the respective line in any of the Table I, application no. 3, columns 5 or 8, preferably the coding region thereof, fragments or homologs thereof. It is preferred that the nucleic acid molecule comprises as little as possible other nucleotides not shown in the respective line in any one of Table I, application no. 3, columns 5 or 8, preferably the coding region thereof, fragments or homologs thereof. In one embodiment, the nucleic acid molecule comprises less than 500, 400, 300, 200, 100, 90, 80, 70, 60, 50 or 40 further nucleotides. In a further embodiment, the nucleic acid molecule comprises less than 30, 20 or 10 further nucleotides. In one embodiment, the nucleic acid molecule used in the process of the invention is identical to the sequences shown in the respective line in Table I, application no. 3, columns 5 or 8, preferably the coding region thereof.

Also preferred is that the nucleic acid molecule used in the process of the invention encodes a polypeptide comprising the sequence shown in the respective line in Table II, application no. 3, columns 5 or 8, fragments or homologs thereof. In one embodiment, the nucleic acid molecule encodes less than 150, 130, 100, 80, 60, 50, 40 or 30 further amino acids. In a further embodiment, the encoded polypeptide comprises less than 20, 15, 10, 9, 8, 7, 6 or 5 further amino acids. In one embodiment used in the inventive process, the encoded polypeptide is identical to the sequences shown in the respective line in Table II, application no. 3, columns 5 or 8.

In one embodiment, the nucleic acid molecule of the invention or used in the process encodes a polypeptide comprising the sequence shown in the respective line in Table II, application no. 3, columns 5 or 8, or fragments or homologs thereof, and comprises less than 100 further nucleotides. In a further embodiment, said nucleic acid molecule comprises less than 30 further nucleotides. In one embodiment, the nucleic acid molecule used in the process is identical to a coding sequence of the sequences shown in the respective line in Table I, application no. 3, columns 5 or 8.

Polypeptides (=proteins), which still have the essential biological or enzymatic activity of the polypeptide of the present invention conferring the production or the increased production of the respective fine chemical 3,4-dihydroxyphenylalanine (dopa), 5-oxoproline, alanine, asparagine, aspartate, citrulline, glycine, homoserine, ornithine, phenylalanine, serine, or tyrosine as compared to a corresponding, e.g. non-transformed, wild type non-human organism, like a microorganism or a plant cell, plant or part thereof, i.e. whose activity is essentially not reduced, are polypeptides with at least 10%, 20%, 30%, 40%, 50%, 60%, 70%, 80%, 90%, 95% or more of the wild type biological activity or enzymatic activity, advantageously, the activity is essentially not reduced in comparison with the activity of a polypeptide shown in the respective line in Table II, application no. 3, columns 5 or 8 expressed under identical conditions.

Homologues of nucleic acid molecules shown in the respective line in Table I, application no. 3, columns 5 or 8, preferably the coding region thereof, or fragments thereof, or of the derived sequences shown in the respective line in Table II, application no. 3, columns 5 or 8, or fragments thereof, also mean truncated sequences, cDNA, single-stranded DNA or RNA of the coding and noncoding DNA sequence. Homologues of said sequences are also understood as meaning derivatives, which comprise noncoding regions such as, for example, UTRs, introns, terminators, enhancers or promoter variants. The promoters upstream of the nucleotide sequences stated can be modified by one or more nucleotide substitution(s), insertion(s) and/or deletion(s) without, however, interfering with the functionality or activity either of the promoters, the open reading frame (=ORF) or with the 3′-regulatory region such as terminators or other 3′-regulatory regions, which are far away from the ORF. It is furthermore possible that the activity of the promoters is increased by modification of their sequence, or that they are replaced completely by more active promoters, even promoters from heterologous organisms.

Appropriate promoters are known to the person skilled in the art and are mentioned herein below.

[0370.1.1.3] to [0379.1.1.3] for the disclosure of these paragraphs see [0370.1.1.1] to [0379.1.1.1] above.

Moreover, a native polypeptide conferring the increase of the respective fine chemical 3,4-dihydroxyphenylalanine (dopa), 5-oxoproline, alanine, asparagine, aspartate, citrulline, glycine, homoserine, ornithine, phenylalanine, serine, or tyrosine in a non-human or ganism or a part thereof can be isolated from cells (e.g., endothelial cells), for example using the antibody of the present invention as described below, in particular, an antibody against proteins having 2,3-dihydroxyphenylpropionate 1,2-dioxygenase, 2-C-methyl-D-erythritol 4-phosphate cytidylyltransferase, 2-oxoglutarate dehydrogenase E1 subunit, 30S ribosomal protein S3, 3-deoxy-7-phosphoheptulonate synthase, 3-deoxy-D-arabinoheptulosonate-7-phosphatesynthase, 3-oxoacyl-[acyl-carrier-protein] synthase, 3-phosphoglycerate dehydrogenase, 47266012-protein, 49747384_SOYBEAN-protein, 50S ribosomal protein L14, 5′-nucleotidase, ABC metal ion transporter substrate-binding protein, ABC transporter ATP binding component, ABC transporter ATP-binding protein, ABC transporter component, ABC transporter permease protein, ABC transporter substrate-binding protein, acetolactate synthase, acetolactate synthase small subunit, acetyl CoA carboxylase, acetyl-CoA acetyltransferase, acetyltransferase, acid shock protein, acyl transferase, acyl-CoA dehydrogenase, acyl-CoA synthase, adenylate kinase, adenylylsulfate kinase, agmatinase, aldehyde dehydrogenase, amine oxidase, amino acid ABC transporter permease protein, amino acid acetyltransferase, amino acid transporter, amino-acid acetyltransferase, aminotransferase, ammonium transporter, ankyrin repeat family protein, anthranilate synthase component I, arginine exporter protein, arginine N-succinyltransferase, argininosuccinate lyase, asparaginase, aspartate 1-decarboxylase, aspartate aminotransferase, aspartate kinase, aspartate-ammonia ligase, At1g17440-protein, At1g19800-protein, At4g32480-protein, At5g16650-protein, at5g21910-protein, ATP-binding component of a transport system, ATP-dependent RNA helicase, auxin response factor, AX653549-protein, AY087308-protein, b0391-protein, b0456-protein, b0518-protein, b0801-protein, b0841-protein, b0917-protein, b1003-protein, b1024-protein, b1137-protein, b1155-protein, b1163-protein, b1179-protein, b1205-protein, b1229-protein, b1259-protein, b1280-protein, b1330-protein, b1445-protein, b1522-protein, b1583-protein, b1670-protein, bl 672-protein, b1837-protein, b1898-protein, b1930-protein, b2032-protein, b2107-protein, b2360-protein, b2399-protein, b2474-protein, b2513-protein, b2613-protein, b2673-protein, b2739-protein, b2812-protein, b2849-protein, b3083-protein, b3098-protein, b3121-protein, b3221-protein, b3246-protein, b3346-protein, b3392-protein, b3410-protein, b3509-protein, b3646-protein, b3791-protein, b3814-protein, b3989-protein, b4029-protein, b4121-protein, beta-galactosidase, beta-hydroxylase, bifunctional aspartokinase/homoserine dehydrogenase, bifunctional purine biosynthesis protein, branched chain aminotransferase, branched-chain amino acid permease, calcium-dependent protein kinase, carbon dioxide concentrating mechanism protein, cation-transporting ATPase, CBL-interacting protein kinase, CDP-diacylglycerol-glycerol-3-phosphate 3-phosphatidyltransferase, CDP-diacylglycerol-serine O-phosphatidyltransferase, CDP-glucose-4,6-dehydratase, cell division control protein, cellulose synthase catalytic subunit, Chaperone protein CIpB, chorismate mutase-T and prephenate dehydrogenase, citrate synthase, coproporphyrinogen III oxidase, CTP synthetase, cullin, cyclin D, cystathionine gamma-synthase, cytochrome c-type protein, D-3-phosphoglycerate dehydrogenase, delta-1-pyrroline 5-carboxylase synthetase, diacylglycerol O-acyltransferase, dihydroxyacid dehydratase, DNA helicase II, DNA mismatch repair protein, DNA-binding protein, DnaJ-like chaperone, D-serine dehydratase, electron transfer flavoprotein subunit beta, elongation factor Tu, enoyl-CoA hydratase, enterobacterial common antigen polymerase, eukaryotic translation initiation factor, eukaryotic translation initiation factor 5, exopolyphosphatase, Fe—S subunit of oxidoreductase, flavodoxin, fumarylacetoacetate hydrolase, gamma-glutamyltranspeptidase, GDP-mannose dehydrogenase, geranylgeranyl pyrophosphate synthase, gibberellin 20-oxidase, Glu/Leu/Phe/Val dehydrogenase , glucokinase, glucose dehydrogenase, glucose-1-phosphate cytidylyltransferase, glucose-6-phosphate 1-dehydrogenase, glutamate-ammonialigase, glutamine amidotransferase, glutamine synthetase, glutaredoxin, glutathione peroxidase, glutathione S-transferase, Glycine cleavage system T aminomethyltransferase, glycogen (starch) synthase, glycogen synthase, glycoprotease, glycosyl transferase, GM02LC11114protein, GM02LC17485-protein, harpin-induced family protein, heat shock transcription factor, HesB/YadR/YfhF family protein, histone acetyltransferase, histone H2A, homocitrate synthase, homoserine O-acetyltransferase, hydrolase, integral membrane transporter family protein, iron(III) dicitrate-binding protein, isochorismate synthase, kinase, L-aspartate oxidase, lipoprotein precursor, lysine decarboxylase, lysyl-tRNA synthetase, major facilitator superfamily transporter protein, malate dehydrogenase, malic enzyme, membrane protein, membrane transport protein, metal ion transporter, metal-dependent hydrolase, methylglyoxal synthase, methylglyoxalase, methyltransferase, mitochondrial inner membrane protease subunit, molecular chaperone portein, monothiol glutaredoxin, monthiol glutaredoxin, multidrug resistance protein, multiple antibiotic resistance protein, murein transglycosylase, N-(5′-phosphoribosyl)anthranilate isomerase, Na+/K+ transporter, N-acetyl-gamma-glutamyl-phosphate reductase, NADH dehydrogenase I chain I, NADH dehydrogenase subunit N, NADH-quinone oxidoreductase subunit, NADP-dependent malic enzyme, nitrate/nitrite transport protein, O-antigen chain length determinant, oxidoreductase, oxidoreductase subunit, oxireductase, peptidase, permease protein of phosphate ABC transporter, phosphate permease, phosphate transporter, phosphoadenosine phosphosulfate reductase , phosphoanhydride phosphorylase, phosphoenolpyruvate carboxykinase, phosphopantetheine adenylyltransferase, phosphoribosylformyl glycinamidine synthase subunit, Photosystem I reaction center subunit XI, photosystem II protein, polygalacturonase, precorrin methylase, Pre-mRNA-splicing factor, prolipoprotein diacylglyceryl transferase, protease, protein kinase, protein phosphatase, PTS system N-acetylmuramic acid-specific EIIBC component, purine biosynthesis protein, pyruvate kinase, restriction alleviation protein, riboflavin biosynthesis protein, RNA binding protein, RNA polymerase sigma factor, RNA-binding protein, s_pp015018333r-protein, sec-independent protein translocase, Sec-independent protein translocase subunit, serine acetyltransferase, serine hydroxymethyltransferase, serine protease, serine/threonine dehydratase, Serine/threonine-protein phosphatase, short-chain alcohol dehydrogenase family, sll0281-protein, sll1032-protein, sll1761-protein, sn-glycerol-3-phosphate dehydrogenase, sodium/proton antiporter, sterol 24-C-methyltransferase, thioredoxin, thioredoxin family protein, threonine dehydratase, threonine synthase, transcription factor, transcriptional regulator, transport protein, trehalose-phosphatase, tryptophan biosynthesis protein, tryptophan synthase alpha chain, TTC0768-protein, TTC0881-protein, TTC1386protein, urease subunit, uridine/cytidine kinase, uroporphyrin-111 C-methyltransferase, XM_{—}473199-protein, ydr183w-protein, ydr273w-protein, ydr507c-protein, yer014w-protein, yer106w-protein, YFL019C-protein, yfl054c-protein, ygl096w-protein, ygl237c-protein, ygr221c-protein, yhl013c-protein, yhr006w-protein, yhr207c-protein, YKL038W-protein, yml083c-protein, ymr013c-protein, YNL086W-protein, yol160w-protein, zinc finger protein, zinc transporter, or Zm_{—}4842_BE510522-protein activity, respectively, or an antibody against polypeptides as shown in the respective line in Table II, application no. 3, columns 5 or 8, or fragments or homologs thereof which can be produced by standard techniques utilizing the polypeptid of the present invention or fragment thereof, i.e., the polypeptide of this invention (FCRP). Preferred are monoclonal antibodies.

for the disclosure of this paragraph see [0381.1.1.1] above.

In one embodiment, the present invention relates to a polypeptide as depicted in the respective line in Table II, especially in Table IIA or especially in Table IIB, application no. 3, columns 5 or 8, or fragment or homolog thereof or as coded by the nucleic acid molecule shown in the respective line in Table I, especially Table IA or especially Table IB, application no. 3, columns 5 or 8, preferably the coding region thereof, or fragment or homolog thereof.

In an embodiment, the present invention relates to a polypeptide comprising or consisting of a consensus sequence shown in the respective line in Table IV, application no. 3, column 8 or at least one polypeptide motif shown in the respective line in Table IV, application no. 3, column 8.

In an embodiment thereof, the present invention relates to a polypeptide comprising or consisting of a consensus sequence shown in the respective line in Table IV, application no. 3, column 8 or comprising at least one polypeptide motif shown in the respective line in Table IV, application no. 3, column 8, whereby not more than 20, 15, 10, 9, 8, 7, 6, 5, 4, 3, 2, or 1, or 0 of the amino acids can be replaced by any amino acid.

In another embodiment thereof, the present invention relates to a polypeptide comprising or consisting of a consensus sequence shown in the respective line in Table IV, application no. 3, column 8 or comprising at least one polypeptide motif shown in the respective line in Table IV, application no. 3, column 8, whereby not more than 15%, 10%, 5%, 4%, 3%, 2% or1% or 0% of the amino acids defined as distinct specific amino acids are/is replaced by another amino acid.

In another embodiment thereof, the present invention relates to a polypeptide comprising or consisting of a consensus sequence shown in the respective line in Table IV, application no. 3, column 8 or comprising at least one polypeptide motif shown in the respective line in Table IV, application no. 3, column 8, whereby not more than 20, 15, 10, 9, 8, 7, 6, 5, 4, 3, 2, or 1, or 0 amino acids are inserted into the consensus sequence or polypeptide motif.

In an embodiment of the present invention, the polypeptide of the present invention comprises or consists of a consensus sequence or at least one polypeptide motif shown in the respective line in Table IV, application no. 3, column 8, whereby said polypeptide distinguishes over the sequence depicted in the respective line in Table II, application no. 3, columns 5 or 8 by one or more amino acids. In one embodiment, said polypeptide of the invention distinguishes over the sequence shown in the respective line in Table II, application no. 3, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 3, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in the respective line in Table II, application no. 3, columns 5 and 8.

In an embodiment, the invention relates to a polypeptide conferring an increase in the respective fine chemical 3,4-dihydroxyphenylalanine (dopa), 5-oxoproline, alanine, asparagine, aspartate, citrulline, glycine, homoserine, ornithine, phenylalanine, serine, or tyrosine in a non-human organism, especially a microorganism or a plant, or a part thereof, being encoded by the nucleic acid molecule of the invention or used in the process of the invention and having a sequence which distinguishes over the sequence as shown in the respective line in Table II, application no. 3, columns 5 or 8 by one or more amino acids (but not exceeding 5%, preferably 4%, 3%, 2%, 1%, 0.5%, 0.2% or 0.1%). In an embodiment, said polypeptide of the invention does not comprise or consist of the sequence shown in the respective line in Table II, application no. 3, columns 5 or 8. In an embodiment, said polypeptide of the present invention is less than 100%, 99.999%, 99.99%, 99.9% or 99% identical. In one embodiment, said polypeptide which differs at least in one or more amino acids (but not exceeding 5%, preferably 4%, 3%, 2%, 1%, 0.5%, 0.2% or 0.1%) from the polypeptide shown in the respective line in Table II, application no. 3, columns 5 and 8 does not comprise a protein of the sequence shown in the respective line in Table II A and/or II B, application no. 3, columns 5 or 8.

In an embodiment, the present invention relates to a polypeptide having the activity of the protein as shown in the respective line in Table II, application no. 3, column 3, which distinguishes over the sequence depicted in the respective line in Table II, application no. 3, columns 5 or 8 by one or more amino acids, preferably by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 3, columns 5 and 8 by not more than 80%, 70%, 60%, 50%, 40%, 30%, 20%, 10%, 5%, 4%, 3%, 2%, 1% or 0.5%.

In another embodiment the polypeptide of the invention takes the form of a preprotein consisting of a plastidial or mitochondrial transit peptide joint to a polypeptide having the activity of the protein as shown in Table II, column 3, from which the transit peptide is preferably cleaved off upon transport of the preprotein into the organelle for example into the plastid or mitochondrion.

In another embodiment the polypeptide of the invention takes not the form of a preprotein.

[0390.1.1.3] to [0391.1.1.3] for the disclosure of these paragraphs see [0390.1.1.1] to [0391.1.1.1] above.

A polypeptide of the invention can participate in the process of the present invention. The polypeptide or a portion thereof comprises preferably an amino acid sequence, which is sufficiently homologous to an amino acid sequence shown in the respective line in Table II A and/or II B, application no. 3, columns 5 or 8.

Further, the polypeptide can have an amino acid sequence which is encoded by a nucleotide sequence which hybridizes, preferably hybridizes under stringent conditions as described above, to a nucleotide sequence of the nucleic acid molecule of the present invention. Accordingly, the polypeptide has an amino acid sequence which is encoded by a nucleotide sequence that is at least about 35%, 40%, 45%, 50%, 55%, 60%, 65%, 70%, 75%, 80%, 85%, 90%, 91%, 92%, 93%, 94%, 95%, 96%, 97%, 98%, 99%, 99.5% or more homologous to one of the amino acid sequences as shown in the respective line in Table II A and/or II B, application no. 3, columns 5 or 8, or fragments thereof. The preferred polypeptide of the present invention preferably possesses at least one of the activities according to the invention and described herein. A preferred polypeptide of the present invention includes an amino acid sequence encoded by a nucleotide sequence which hybridizes, preferably hybridizes under stringent conditions, to a nucleotide sequence shown in the respective line in Table I A and/or I B, application no. 3, columns 5 or 8, preferably the coding region thereof, or fragments thereto or which is homologous thereto, as defined above

The polypeptide of the present invention can vary from the sequences shown in the respective line in Table II A and/or II B, application no. 3, columns 5 or 8 in amino acid sequence due to natural variation or mutagenesis, as described in detail herein. Accordingly, the polypeptide comprise an amino acid sequence which is at least about 35%, 40%, 45%, 50%, 55%, 60%, 65%, 70%, 75%, 80%, 85% or 90, 91%, 92%, 93%, 94%, 95%, 96%, 97%, 98%, 99%, 99.5% or more homologous to an entire amino acid sequence shown in the respective line in Table II A and/or II B, application no. 3, columns 5 or 8.

Biologically active portions of a polypeptide of the present invention include peptides comprising amino acid sequences derived from the amino acid sequence of the polypeptide of the present invention or used in the process of the present invention, e.g., the amino acid sequence shown in the respective line in Table II, application no. 3, columns 5 or 8 or the amino acid sequence of a protein homologous thereto, which include fewer amino acids than a full length polypeptide of the present invention or used in the process of the present invention or the full length protein which is homologous to a polypeptide of the present invention or used in the process of the present invention depicted herein, and exhibit at least one activity of a polypeptide of the present invention or used in the process of the present invention.

for the disclosure of this paragraph see [0396.1.1.1] above.

Manipulation of the nucleic acid molecule of the invention may result in the production of a protein having differences from the protein as shown in the respective line in Table II, application no. 3, column 5 or 8. Differences shall mean at least one amino acid different from the sequences as shown in the respective line in Table II, application no. 3, column 5 or 8, preferably at least 2, 3, 4, 5, 6, 7, 8, 9 or 10 amino acids (especially but not exceeding 0.1%, preferably 0.2%, 0.5%, 1%, 2%, 3% 4% or 5%), more preferably at least 15, 20, 25, 30, 35, 40, 45 or 50 amino acids (especially but not exceeding 0.5%, preferably 1%, 2%, 3% 4%, 5%, 10% or 25%) different from the sequences as shown in the respective line in Table II, application no. 3, column 5 or 8. These proteins may be improved in efficiency or activity, or may be more stable and therefore present in greater numbers in the cell as compared to the wild-type cell.

Any mutagenesis strategies for the polypeptide of the present invention or the polypeptide used in the process of the present invention to result in increasing said activity are not meant to be limiting; variations on these strategies will be readily apparent to one skilled in the art. Using such strategies, and incorporating the mechanisms disclosed herein, the nucleic acid molecule and polypeptide of the invention may be utilized to generate algae, ciliates, fungi, other microorganisms like C. glutamicum or plants or parts thereof, expressing proteins as shown in the respective line in Table II, application no. 3, column 5 or 8, or mutated proteins thereof. The nucleic acid molecules and polypeptide molecules of the invention are expressed such that the yield, production, and/or efficiency of production of a desired compound is improved.

[0399.1.1.3] to [0401.1.1.3] for the disclosure of these paragraphs see [0399.1.1.1] to [0401.1.1.1] above.

In one embodiment, a protein (=polypeptide) as shown in the respective line in Table II, application no. 3, column 5 or 8 refers to a polypeptide having an amino acid sequence corresponding to the polypeptide of the invention, especially to a polypeptide as shown in Table II, application no. 3, columns 5 or 8 or homologs thereof, or used in the process of the invention, whereas a “non- inventive protein or polypeptide” or “other polypeptide” refers to a polypeptide having an amino acid sequence corresponding to a protein which is not substantially homologous to a polypeptide of the invention, preferably which is not substantially homologous to a polypeptide or protein as shown in the respective line in Table II, application no. 3, column 5 or 8 or homologs thereof, e.g., a protein which does not confer the activity described herein and which is derived from the same or a different organism.

[0403.1.1.3] to [0409.1.1.3] for the disclosure of these paragraphs see [0403.1.1.1] to [0409.1.1.1] above.

As the nucleic acid molecules of the invention and/or the expression thereof is related to the synthesis of the respective fine chemical 3,4-dihydroxyphenylalanine (dopa), 5-oxoproline, alanine, asparagine, aspartate, citrulline, glycine, homoserine, ornithine, phenylalanine, serine, or tyrosine its function as a probe extends to the detection of microorganisms, plant tissues, plants, plant variets, plant ecotypes or plant genera with varying, advantageously increased, capability or potential for synthesis of the respective fine chemical 3,4-dihydroxyphenylalanine (dopa), 5-oxoproline, alanine, asparagine, aspartate, citrulline, glycine, homoserine, ornithine, phenylalanine, serine, or tyrosine. Therefore in one embodiment the present invention relates to a method for analyzing the capability or potential of a plant tissue, a plant, a plant variety or ecotype to produce the respective fine chemical 3,4-dihydroxyphenylalanine (dopa), 5-oxoproline, alanine, asparagine, aspartate, citrulline, glycine, homoserine, ornithine, phenylalanine, serine, or tyrosine by using the respective antibody of the invention as a probe to detect the amount of the polypeptide encoded by said nucleic acid molecule of the invention in a non-human organism in comparison to another organism.

Therefore in one embodiment the invention relates to a method to probe a non-human organism for its capability for the production of the respective fine chemical comprising the steps of
a) analyzing the sequence and/or the expression of the respective nucleic acid molecule of the invention in least two non-human organisms which differ in the capability to produce the respective fine chemical;
b) associate the different sequence and/or expression level of the nucleic acid molecules of the invention with their different capability to produce the respective fine chemical;
c) analyze the sequence and/or the expression of the nucleic acid molecule of the invention in at least a third non-human organism;
d) predict the capability of the at least third organism to produce the respective fine chemical based by comparing the sequence and/or expression of the nucleic acid of the invention to the sequences and/or expression levels of the nucleic acid molecule of the invention of the at least two organisms analyzed in step a.

[0411.1.1.3] to [0430.1.1.3] for the disclosure of these paragraphs see [0411.1.1.1] to [0430.1.1.1] above.

Accordingly the present invention relates to any cell transgenic for any nucleic acid characterized as part of the invention, e.g. conferring the increase of the respective fine chemical 3,4-dihydroxyphenylalanine (dopa), 5-oxoproline, alanine, asparagine, aspartate, citrulline, glycine, homoserine, ornithine, phenylalanine, serine, or tyrosine in a cell or a non-human organism or a part thereof, e.g. the nucleic acid molecule of the invention, the nucleic acid construct of the invention, the vector of the invention, the expression cassette according to the invention, or a nucleic acid molecule encoding the polypeptide of the invention, e.g. encoding a polypeptide having an activity as the protein as shown in the respective line in Table II, application no. 3, column 3. Due to the above-mentioned activity the respective fine chemical 3,4-dihydroxyphenylalanine (dopa), 5-oxoproline, alanine, asparagine, aspartate, citrulline, glycine, homoserine, ornithine, phenylalanine, serine, or tyrosine content in a cell or a non-human organism is increased. For example, due to modulation or manipulation, the cellular activity is increased, in a preferred embodiment in organelles such as plastids or mitochondria, e.g. due to an increased expression or specific activity or specific targeting of the subject matters of the invention in a cell or a non-human organism or a part thereof especially in organelles such as plastids or mitochondria, or in another embodiment in the cytosol. Transgenic for a polypeptide having a protein or a protein activity means herein that due to modulation or manipulation of the genome, the activity of protein as shown in the respective line in Table II, application no. 3, column 3 or a protein as shown in the respective line in Table II, application no. 3, column 3-like activity is increased in the cell or non-human organism or part thereof, especially in organelles such as plastids or mitochondria, or especially in the cytosol. Examples are described above in context with the process of the invention.

for the disclosure of this paragraph see [0432.1.1.1] above.

A naturally occurring expression cassette—for example the naturally occurring combination of the promoter of the gene encoding a protein as shown in the respective line in Table II, application no. 3, column 5 or 8, preferably the coding region thereof, or fragments or homologs thereof with the corresponding encoding gene—becomes a transgenic expression cassette when it is modified by non-natural, synthetic “artificial” methods such as, for example, mutagenesis. Such methods have been described (U.S. Pat. No. 5,565,350; WO 00/15815; also see above).

[0434.1.1.3] to [0435.1.1.3] for the disclosure of these paragraph see [0434.1.1.1] to [0435.1.1.1] above.

Transgenic plants comprising the respective fine chemical(s) synthesized in the process according to the invention can be marketed directly without isolation of the fine chemical synthesized. In the process according to the invention, plants are understood as meaning all plant parts, plant organs such as leaf, stalk, root, tubers or seeds or propagation material or harvested material or the intact plant. In this context, the seed encompasses all parts of the seed such as the seed coats, epidermal cells, seed cells, endosperm or embryonic tissue. The fine chemical produced in the process according to the invention may, however, also be isolated from the plant. In case of 3,4-dihydroxyphenylalanine (dopa), 5-oxoproline, alanine, asparagine, aspartate, citrulline, glycine, homoserine, ornithine, phenylalanine, serine, or tyrosine this can be in free form or bound to proteins. Fine chemical(s) produced by this process can be harvested by harvesting the non-human organisms either from the culture in which they grow or from the field. For example, this can be done via squeezing, grinding and/or extraction, salt precipitation and/or ion-exchange chromatography of the plant parts, preferably the plant seeds, plant fruits, plant tubers and the like.

[0437.1.1.3] to [0440.1.1.3] for the disclosure of these paragraphs see [0437.1.1.1] to [0440.1.1.1] above.

[0442.1.1.3] to [0454.1.1.3] for the disclosure of these paragraphs see [0442.1.1.1] to [0454.1.1.1] above.

In one embodiment, the present invention relates to a method for the identification of a gene product conferring the production of or an increase in the fine chemical production in a non-human cell, comprising the following steps:
(a) contacting, e.g. hybridizing, a, some or all nucleic acid molecules of a sample, e.g. cells, tissues, plants or microorganisms or a nucleic acid library, which can contain a candidate gene encoding a gene product conferring the production of or an increase in the fine chemical after expression, with the nucleic acid molecule of the present invention as shown in the respective line in column 5 or 8 of Table I A or B of application no. 3, preferably the coding region thereof, or a fragment or homolog thereof;
(b) identifying the nucleic acid molecules, which hybridize under relaxed stringent conditions with the nucleic acid molecule of the present invention in particular to the nucleic acid molecule sequence shown in the respective line in Table I, application no. 3, columns 5 or 8, preferably the coding region thereof, of a fragment or a homolog thereof, preferably in Table I B, application no. 3, columns 5 or 8, preferably the coding region thereof, of a fragment or a homolog thereof, and, optionally, isolating the full length cDNA clone or complete genomic clone;
(c) introducing the candidate nucleic acid molecules or a fragment thereof in host cells, preferably in a plant cell or a microorganism, appropriate for producing the fine chemical;
(d) expressing the identified nucleic acid molecules in the host cells for which the production of or the increased production of the fine chemical is desired;
(e) assaying the fine chemical level in the non-human host cells; and
(f) identifying the nucleic acid molecule and/or its gene product which expression confers the production of or an increase in the fine chemical level in the non-human host cell after expression compared to the wild type.

for the disclosure of this paragraph see [0456.1.1.1] above.

In another embodiment, the present invention relates to a method for the identification of a gene product conferring the production of or an increase in the fine chemical production in a cell, comprising the following steps:
(a) identifying a nucleic acid molecule of a non-human organism, which is at least 20%, 25%, 30%, 35%, 40%, 50%, 60%, 70%, 80%, 90%, 95%, 96%, 97%, 98%, 99%, 99.5% or more homolog to the nucleic acid molecule encoding a polypeptide comprising the polypeptide molecule as shown in the respective line in column 5 or 8 of Table II, application no. 3, or comprising a consensus sequence or a polypeptide motif as shown in the respective line in column 8 of Table IV, application no. 3, or being encoded by a nucleic acid molecule comprising a polynucleotide as shown in the respective line in column 5 or 8 of Table I, application no. 3, preferably the coding region thereof, or a homologue thereof as described herein, for example via homology search in a data bank;
(b) introducing the candidate nucleic acid molecule in non-human host cells, preferably in a plant cell or a microorganism, appropriate for producing the fine chemical;
(c) expressing the identified nucleic acid molecule in the non-human host cells;
(d) assaying the fine chemical level in the non-human host cells; and
(e) identifying the nucleic acid molecule and its gene product which expression confers the production of or an increase in the fine chemical level in the non-human host cell after expression compared to the wild type.

for the disclosure of this paragraph see [0458.1.1.1] above.

Further, the nucleic acid molecules disclosed herein, in particular the nucleic acid molecules shown in the respective line in column 5 or 8 of Table I A or B of application no. 3, may be sufficiently homologous to the sequences of related species such that these nucleic acid molecules may serve as markers for the construction of a genomic map in related organism or for association mapping. Furthermore natural variation in the genomic regions corresponding to nucleic acids disclosed herein, in particular the nucleic acid molecule shown in the respective line in column 5 or 8 of Table I A or B, of application no. 3 or homologous thereof, may lead to variation in the activity of the proteins disclosed herein, in particular the proteins comprising polypeptides as shown in the respective lines in column 5 or 8 of Table II A or B, of application no. 3 or their homologous, or comprising the consensus sequence or the polypeptide motif as shown in the respective line in column 8 of Table IV, and in consequence in a natural variation of an increased fine chemical production.

In consequence natural variation eventually also exists in form of more active allelic variants leading already to a relative increase in the fine chemical. Different variants of the nucleic acid molecules disclosed herein, in particular the nucleic acid molecules comprising the nucleic acid molecules as shown in the respective line in column 5 or 8 of Table I A or B of application no. 3, preferably the coding region thereof, which corresponds to different levels of increase in fine chemical, e.g. different levels of increase in fine chemical, can be indentified and used for marker assisted breeding for an enhanced production of the fine chemical.

Natural variation may also exist in the regulatory regions, e.g. the promoters of the natural genes comprising the nucleic acid molecules as shown in the respective line column 5 or 8 of Table I A or B of application no. 3, preferably the coding region thereof, which can similarly correspond to different levels of increase in the fine chemical, e.g. different levels of increase in the fine chemical can be identified and used for marker assisted breeding for an enhanced production of the fine chemical.

Accordingly, the present invention relates to a method for breeding plants for the production of the fine chemical, comprising
(a) selecting a first plant variety with an increased production of the fine chemical, e.g. based on an increased expression of a nucleic acid molecule of the invention as disclosed herein, in particular of a nucleic acid molecule comprising a nucleic acid molecule as shown in the respective line in column 5 or 8 of Table I A or B of application no. 3, preferably the coding region thereof, or fragments or homologs thereof, or a polypeptide comprising a polypeptide as shown in the respective line in column 5 or 8 of Table II A or B of application no. 3, or fragments or homologs thereof, or comprising a consensus sequence or a polypeptide motif as shown in the respective line in column 8 of Table IV, or a homolog thereof, as described herein;
(b) associating the level of increased production of the fine chemical, with the expression level or the genomic structure of a gene encoding said polypeptide or said nucleic acid molecule;
(c) crossing the first plant variety with a second plant variety, which significantly differs in its level of fine chemical; and
(d) identifying, which of the offspring varieties has got increased production of the fine chemical by the expression level of said polypeptide or nucleic acid molecule or the genomic structure of the gene(s) encoding said polypeptide or nucleic acid molecule of the invention.

[0462.1.1.3] to [0482.1.1.3] for the disclosure of these paragraphs see [0462.1.1.1] to [0482.1.1.1] above.

A special embodiment of this invention is related to genes which increase or generate the production of the fine chemical aspartate, asparagine, alanine, citrulline or ornithine in plant cells, plants or part thereof. Phenotypes thereto are associated with yield of plants (=yield related phenotypes). In accordance with the invention, therefore, the respective genes identified in Table I, wherein in column 7 aspartate, asparagine, alanine, citrulline or ornithine, respectively, are mentioned, especially the coding region thereof, or homologs or fragments thereof, may be employed to enhance any yield-related phenotype. Increased yield may be determined in field trials of transgenic plants and suitable control plants. Alternatively, a transgene's ability to increase yield may be determined in a model plant. An increased yield phenotype may be determined in the field test or in a model plant by measuring any one or any combination of the following phenotypes, in comparison to a control plant: yield of dry harvestable parts of the plant, yield of dry aerial harvestable parts of the plant, yield of underground dry harvestable parts of the plant, yield of fresh weight harvestable parts of the plant, yield of aerial fresh weight harvestable parts of the plant yield of underground fresh weight harvestable parts of the plant, yield of the plant's fruit (both fresh and dried), grain dry weight, yield of seeds (both fresh and dry), and the like.

The most basic yield-related phenotype is increased yield associated with the presence of the gene or a homolog or a fragment thereof as a transgene in the plant, i.e., the intrinsic yield of the plant. Intrinsic yield capacity of a plant can be, for example, manifested in a field test or in a model system by demonstrating an improvement of seed yield (e.g. in terms of increased seed/grain size, increased ear number, increased seed number per ear, improvement of seed filling, improvement of seed composition, embryo and/or endosperm improvements, and the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under non-stressed conditions), improvement in plant architecture.

Increased yield-related phenotypes may also be measured to determine tolerance to abiotic environmental stress. Abiotic stresses include drought, low temperature, salinity, osmotic stress, shade, high plant density, mechanical stresses, and oxidative stress, and yield-related phenotypes are encompassed by tolerance to such abiotic stresses. Additional phenotypes that can be monitored to determine enhanced tolerance to abiotic environmental stress include, without limitation, wilting; leaf browning; loss of turgor, which results in drooping of leaves or needles stems, and flowers; drooping and/or shedding of leaves or needles; the leaves are green but leaf angled slightly toward the ground compared with controls; leaf blades begun to fold (curl) inward; premature senescence of leaves or needles; loss of chlorophyll in leaves or needles and/or yellowing. Any of the yield-related phenotypes described above may be monitored in field tests or in model plants to demonstrate that a transgenic plant has increased tolerance to abiotic environmental stress. In accordance with the invention, the respective genes identified in Table 1, in case in column 7 is indicated, especially the coding region thereof, or homologs or fragments thereof, wherein in the same line aspartate, asparagine, alanine, citrulline or ornithine, respectively, may be employed to enhance tolerance to abiotic environmental stress in a plant, when confronted with abiotic environmental stress.

A polypeptide conferring an yield-increasing activity can be encoded by a respective nucleic acid sequence as shown in Table I, column 5 or 8, and/or comprises or consists of a respective polypeptide as depicted in Table II, column 5 and 8, and/or can be amplified with the respective primer set shown in Table III, column 8, in case in column 7 “aspartate”, “asparagine”, “alanine”, “citrulline” or “ornithine” is indicated.

“Improved adaptation” to environmental stress like e.g. freezing and/or chilling temperatures refers to an improved plant performance under environmental stress conditions.

A modification, i.e. an increase, can be caused by endogenous or exogenous factors. For example, an increase in activity in an organism or a part thereof can be caused by adding a gene product or a precursor or an activator or an agonist to the media or nutrition or can be caused by introducing said subjects into a organism, transient or stable. Furthermore such an increase can be reached by the introduction of the respective inventive nucleic acid sequence or the encoded protein in the correct cell compartment for example into the nucleus or cytoplasmic respectively or into plastids either by transformation and/or targeting.

The term “yield” as used herein generally refers to a measurable produce from a plant, particularly a crop. Yield and yield increase (in comparison to a non-transformed starting or wild-type plant) can be measured in a number of ways, and it is understood that a skilled person will be able to apply the correct meaning in view of the particular embodiments, the particular crop concerned and the specific purpose or application concerned. The terms “improved yield” or “increased yield” can be used interchangeable.

As used herein, the term “improved yield” or the term “increased yield” means any improvement in the yield of any measured plant product, such as grain, fruit or fiber. In accordance with the invention, changes in different phenotypic traits may improve yield. For example, and without limitation, parameters such as floral organ development, root initiation, root biomass, seed number, seed weight, harvest index, tolerance to abiotic environmental stress, leaf formation, phototropism, apical dominance, and fruit development, are suitable measurements of improved yield. Any increase in yield is an improved yield in accordance with the invention. For example, the improvement in yield can comprise a 0.1%, 0.5%, 1%, 3%, 5%, 10%, 15%, 20%, 30%, 40%, 50%, 60%, 70%, 80%, 90% or greater increase in any measured parameter. For example, an increase in the bushel/acre yield of soybeans or corn derived from a crop comprising plants which are transgenic for the nucleotides and polypeptides of Table I, in case in column 7 aspartate, asparagine, alanine, citrulline or ornithine, respectively, is mentioned, as compared with the bushel/acre yield from untreated soybeans or corn cultivated under the same conditions, is an improved yield in accordance with the invention. The increased or improved yield can be achieved in the absence or presence of stress conditions.

For example, enhanced or increased “yield” refers to one or more yield parameters selected from the group consisting of biomass yield, dry biomass yield, aerial dry biomass yield, underground dry biomass yield, fresh-weight biomass yield, aerial fresh-weight biomass yield, underground fresh-weight biomass yield; enhanced yield of harvestable parts, either dry or freshweight or both, either aerial or underground or both; enhanced yield of crop fruit, either dry or fresh-weight or both, either aerial or underground or both; and preferably enhanced yield of seeds, either dry or fresh-weight or both, either aerial or underground or both.

Crop yield is defined herein as the number of bushels of relevant agricultural product (such as grain, forage, or seed) harvested per acre. Crop yield is impacted by abiotic stresses, such as drought, heat, salinity, and cold stress, and by the size (biomass) of the plant. Traditional plant breeding strategies are relatively slow and have in general not been successful in conferring increased tolerance to abiotic stresses. Grain yield improvements by conventional breeding have nearly reached a plateau in maize.

Accordingly, the yield of a plant can depend on the specific plant/crop of interest as well as its intended application (such as food production, feed production, processed food production, biofuel, biogas or alcohol production, or the like) of interest in each particular case. Thus, in one embodiment, yield is calculated as harvest index (expressed as a ratio of the weight of the respective harvestable parts divided by the total biomass), harvestable parts weight per area (acre, square meter, or the like); and the like. The harvest index, i.e., the ratio of yield biomass to the total cumulative biomass at harvest, in maize has remained essentially unchanged during selective breeding for grain yield over the last hundred years. Accordingly, recent yield improvements that have occurred in maize are the result of the increased total biomass production per unit land area. This increased total biomass has been achieved by increasing planting density, which has led to adaptive phenotypic alterations, such as a reduction in leaf angle, which may reduce shading of lower leaves, and tassel size, which may increase harvest index. Harvest index is relatively stable under many environmental conditions, and so a robust correlation between plant size and grain yield is possible. Plant size and grain yield are intrinsically linked, because the majority of grain biomass is dependent on current or stored photosynthetic productivity by the leaves and stem of the plant. As with abiotic stress tolerance, measurements of plant size in early development, under standardized conditions in a growth chamber or greenhouse, are standard practices to measure potential yield advantages conferred by the presence of a transgene.

Acccordingly, the yield of a plant can be increased by improving one or more of the yield-related phenotypes.

Such yield-related phenotypes or traits of a plant the improvement of which results in increased yield comprise, without limitation, the increase of the intrinsic yield capacity of a plant and/or increased stress tolerance, e.g. improved nutrient use efficiency.

For example, yield refers to biomass yield, e.g. to dry weight biomass yield and/or fresh-weight biomass yield. Biomass yield refers to the aerial or underground parts of a plant, depending on the specific circumstances (test conditions, specific crop of interest, application of interest, and the like). In one embodiment, biomass yield refers to the aerial and underground parts. Biomass yield may be calculated as fresh-weight, dry weight or a moisture adjusted basis. Biomass yield may be calculated on a per plant basis or in relation to a specific area (e.g. biomass yield per acre/square meter/or the like).

In other embodiment, “yield” refers to seed yield which can be measured by one or more of the following parameters: number of seeds or number of filled seeds (per plant or per area (acre/square meter/or the like)); seed filling rate (ratio between number of filled seeds and total number of seeds); number of flowers per plant; seed biomass or total seeds weight (per plant or per area (acre/square meter/or the like); thousand kernel weight (TKW; extrapolated from the number of filled seeds counted and their total weight; an increase in TKW may be caused by an increased seed size, an increased seed weight, an increased embryo size, and/or an increased endosperm). Other parameters allowing to measure seed yield are also known in the art. Seed yield may be determined on a dry weight or on a fresh weight basis, or typically on a moisture adjusted basis, e.g. at 15.5 percent moisture.

For example, the term “increased yield” means that the a plant, exhibits an increased growth rate, under conditions of abiotic environmental stress, compared to the corresponding wild-type plant.

An increased growth rate may be reflected inter alia by or confers an increased biomass production of the whole plant, or an increased biomass production of the aerial parts of a plant, or by an increased biomass production of the underground parts of a plant, or by an increased biomass production of parts of a plant, like stems, leaves, blossoms, fruits, and/or seeds.

Increased yield includes higher fruit yields, higher seed yields, higher fresh matter production, and/or higher dry matter production.

The term “increased yield” means that the plant, exhibits an prolonged growth under conditions of abiotic environmental stress, as compared to the corresponding, e.g. non-transformed, wild type organism. A prolonged growth comprises survival and/or continued growth of the plant, at the moment when the non-transformed wild type organism shows visual symptoms of deficiency and/or death.

When the plant of the invention is a corn plant increased yield for corn plants means, for example, increased seed yield, in particular for corn varieties used for feed or food. Increased seed yield of corn refers to an increased kernel size or weight, an increased kernel per ear, or increased ears per plant. Alternatively or in addition the cob yield may be increased, or the length or size of the cob is increased, or the kernel per cob ratio is improved.

When the plant of the invention is a soy plant, increased yield for soy plants means increased seed yield, in particular for soy varieties used for feed or food. Increased seed yield of soy refers for example to an increased kernel size or weight, an increased kernel per pod, or increased pods per plant.

When the plant of the invention is an oil seed rape (OSR) plant, increased yield for OSR plants means increased seed yield, in particular for OSR varieties used for feed or food. Increased seed yield of OSR refers to an increased seed size or weight, an increased seed number per silique, or increased siliques per plant.

When the plant of the invention is a cotton plant. Increased yield for cotton plants means increased lint yield. Increased lint yield of cotton refers in one embodiment to an increased length of lint.

Said increased yield can typically be achieved by enhancing or improving, one or more yieldrelated traits of the plant. Such yield-related traits of a plant comprise, without limitation, the increase of the intrinsic yield capacity of a plant, and/or increased stress tolerance, in particular increased abiotic stress tolerance, like improved nutrient use efficiency, e.g. nitrogen use efficiency.

Intrinsic yield capacity of a plant can be, for example, manifested by improving the specific (intrinsic) seed yield (e.g. in terms of increased seed/ grain size, increased ear number, increased seed number per ear, improvement of seed filling, improvement of seed composition, embryo and/or endosperm improvements, or the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under stressed or non-stressed conditions), improvement in plant architecture, cell cycle modifications, photosynthesis modifications, various signaling pathway modifications, modification of transcriptional regulation, modification of translational regulation, modification of enzyme activities, and the like); and/or the like.

The improvement or increase of stress tolerance of a plant can for example be manifested by improving or increasing a plant's tolerance against stress, particularly abiotic stress. In the present application, abiotic stress refers generally to abiotic environmental conditions a plant is typically confronted with, including, but not limited to, drought (tolerance to drought may be achieved as a result of improved water use efficiency), heat, low temperatures and cold conditions (such as freezing and chilling conditions), nutrient depetion, salinity, osmotic stress , shade, high plant density, mechanical stress, oxidative stress, and the like.

The increased plant yield can for example be mediated by increasing the “nutrient use efficiency of a plant”, e.g. by improving the use efficiency of nutrients including, but not limited to, phosphorus, potassium, and nitrogen. Further, higher yields may be obtained with current or standard levels of nitrogen use.

Generally, the term “increased tolerance to stress” can be defined as survival of plants, and/or higher yield production, under stress conditions as compared to a non-transformed wild type or starting plant: For example, the plant of the invention or produced according to the method of the invention is better adapted to the stress conditions. “Improved adaptation” to environmental stress like e.g. drought, heat, nutrient depletion, freezing and/or chilling temperatures refers herein to an improved plant performance resulting in an increased yield, particularly with regard to one or more of the yield related traits as defined in more detail above.

During its life-cycle, a plant is generally confronted with a diversity of environmental conditions. Any such conditions, which may, under certain circumstances, have an impact on plant yield, are herein referred to as “stress” condition. Environmental stresses may generally be divided into biotic and abiotic (environmental) stresses. Unfavorable nutrient conditions are also referred to as “environmental stress”. The present invention does also contemplate solutions for this kind of environmental stress, e.g. referring to increased nutrient use efficiency.

For the purposes of the description of the present invention, the terms “enhanced tolerance to abiotic stress”, “enhanced resistance to abiotic environmental stress”, “enhanced tolerance to environmental stress”, “improved adaptation to environmental stress” and other variations and expressions similar in its meaning are used interchangeably and refer, without limitation, to an improvement in tolerance to one or more abiotic environmental stress(es) as described herein and as compared to a corresponding origin or wild type plant or a part thereof.

The term abiotic stress tolerance(s) refers for example low temperature tolerance, drought tolerance or improved water use efficiency (WUE), heat tolerance, improved nutrient efficiency, especially improved nitrogen use efficiency (NUE), salt stress tolerance and others. Studies of a plant's response to desiccation, osmotic shock, and temperature extremes are also employed to determine the plant's tolerance or resistance to abiotic stresses. Water use efficiency (WUE) is a parameter often correlated with drought tolerance. In selecting traits for improving crops, a decrease in water use, without a change in growth would have particular merit in an irrigated agricultural system where the water input costs were high. An increase in growth without a corresponding jump in water use would have applicability to all agricultural systems. In many agricultural systems where water supply is not limiting, an increase in growth, even if it came at the expense of an increase in water use also increases yield.

Drought stress means any environmental stress which leads to a lack of water in plants or reduction of water supply to plants, including a secondary stress by low temperature and/or salt, and/or a primary stress during drought or heat, e.g. desiccation etc.

### Specific Embodiments

Accordingly, this invention provides respective measures and methods to produce plants with increased yield, e.g. genes conferring an increased yield-related trait, for example enhanced tolerance to abiotic environmental stress, for example an increased drought tolerance and/or low temperature tolerance and/or an increased nutrient use efficiency, intrinsic yield and/or another increased yield-related trait, upon expression or over-expression. Accordingly, the present invention provides such genes in case in column 7 of the respective Table “aspartate” or “alanine” is indicated. In particular, such genes are described in column 5 as well as in column 8 of Tables I, especially the coding region thereof, or homologs or fragments thereof, in case aspartate or alanine are indicated in column 7 or the respective polypeptides are described in column 5 as well as in column 8 of Table II, or homologs or fragments thereof, in case aspartate or alanine are indicated in column 7.

Accordingly, the present invention provides respective transgenic plants showing one or more improved yield-related traits as compared to the corresponding origin or the wild type plant and methods for producing such transgenic plants with increased yield in case in column 7 of the respective Table aspartate, asparagine, alanine, citrulline or ornithine, respectively is indicated.

In one embodiment, one or more of said yield-increasing activities are increased by increasing the amount and/or the specific activity of one or more proteins listed in Table I, column 5 or 8 in a compartment of a cell indicated in Table I, column 6, in case in column 7 of the respective Table aspartate, asparagine, alanine, citrulline or ornithine is indicated.

Accordingly to present invention, the yield of the plant of the invention is increased by improving one or more of the yield-related traits as defined herein. Said increased yield in accordance with the present invention can typically be achieved by enhancing or improving, in comparison to an origin or wild-type plant, one or more yield-related traits of said plant. Such yield-related traits of a plant the improvement of which results in increased yield comprise, without limitation, the increase of the intrinsic yield capacity of a plant and/or increased stress tolerance, especially increased abiotic stress tolerance.

In a particular embodyment the present the invention relates to plants with a increased nutrient efficiency, especially a nitrogen use efficiency in case a plant is transformed with a gene being depited in Table I, and wherein in the same line of said gene in column 7 aspartate, asparagine, alanine, citrulline or ornithine is indicated.

In a further embodiment the present invention is characterized by the features as disclosed in the following items:

Item 1. A process for the production of a fine chemical selected from the group consisting of: 3,4-dihydroxyphenylalanine (dopa), 5-oxoproline, alanine, asparagine, aspartate, citrulline, glycine, homoserine, ornithine, phenylalanine, serine and tyrosine, which comprises
(a) increasing or generating one or more activities selected from the group consisting of 2,3-dihydroxyphenylpropionate 1,2-dioxygenase, 2-C-methyl-D-erythritol 4-phosphate cytidylyltransferase, 2-oxoglutarate dehydrogenase E1 subunit, 30S ribosomal protein S3, 3-deoxy-7-phosphoheptulonate synthase, 3-deoxy-D-arabinoheptulosonate-7-phosphatesynthase, 3-oxoacyl-[acyl-carrier-protein] synthase, 3-phosphoglycerate dehydrogenase, 47266012-protein, 49747384_SOYBEAN-protein, 50S ribosomal protein L14, 5′-nucleotidase, ABC metal ion transporter substrate-binding protein, ABC transporter ATP binding component, ABC transporter ATP-binding protein, ABC transporter component, ABC transporter permease protein, ABC transporter substrate-binding protein, acetolactate synthase, acetolactate synthase small subunit, acetyl CoA carboxylase, acetyl-CoA acetyltransferase, acetyltransferase, acid shock protein, acyl transferase, acyl-CoA dehydrogenase, acyl-CoA synthase, adenylate kinase, adenylylsulfate kinase, agmatinase, aldehyde dehydrogenase, amine oxidase, amino acid ABC transporter permease protein, amino acid acetyltransferase, amino acid transporter, amino-acid acetyltransferase, aminotransferase, ammonium transporter, ankyrin repeat family protein, anthranilate synthase component I, arginine exporter protein, arginine N-succinyltransferase, argininosuccinate lyase, asparaginase, aspartate 1-decarboxylase, aspartate aminotransferase, aspartate kinase, aspartate-ammonia ligase, At1g17440-protein, At1g19800-protein, At4g32480-protein, At5g16650-protein, at5g21910-protein, ATP-binding component of a transport system, ATP-dependent RNA helicase, auxin response factor, AX653549-protein, AY087308-protein, b0391-protein, b0456-protein, b0518-protein, b0801-protein, b0841-protein, b0917-protein, b1003-protein, b1024-protein, b1137-protein, b1155-protein, b1163-protein, b1179-protein, b1205-protein, b1229-protein, b1259-protein, b1280-protein, b1330-protein, b1445-protein, b1522-protein, b1583-protein, b1670-protein, b1672-protein, b1837-protein, b1898-protein, b1930-protein, b2032-protein, b2107-protein, b2360-protein, b2399-protein, b2474-protein, b2513-protein, b2613-protein, b2673-protein, b2739-protein, b2812-protein, b2849-protein, b3083-protein, b3098-protein, b3121-protein, b3221-protein, b3246-protein, b3346-protein, b3392-protein, b3410-protein, b3509-protein, b3646-protein, b3791-protein, b3814-protein, b3989-protein, b4029-protein, b4121-protein, beta-galactosidase, beta-hydroxylase, bifunctional aspartokinase/homoserine dehydrogenase, bifunctional purine biosynthesis protein, branched chain aminotransferase, branched-chain amino acid permease, calcium-dependent protein kinase, carbon dioxide concentrating mechanism protein, cation-transporting ATPase, CBL-interacting protein kinase, CDP-diacylglycerol-glycerol-3-phosphate 3-phosphatidyltransferase, CDP-diacylglycerol-serine O-phosphatidyltransferase, CDP-glucose-4,6-dehydratase, cell division control protein, cellulose synthase catalytic subunit, Chaperone protein CIpB, chorismate mutase-T and prephenate dehydrogenase, citrate synthase, coproporphyrinogen III oxidase, CTP synthetase, cullin, cyclin D, cystathionine gamma-synthase, cytochrome c-type protein, D-3-phosphoglycerate dehydrogenase, delta 1-pyrroline 5-carboxylase synthetase, diacylglycerol O-acyltransferase, dihydroxyacid dehydratase, DNA helicase II, DNA mismatch repair protein, DNA-binding protein, DnaJ-like chaperone, D-serine dehydratase, electron transfer flavoprotein subunit beta, elongation factor Tu, enoyl-CoA hydratase, enterobacterial common antigen polymerase, eukaryotic translation initiation factor, eukaryotic translation initiation factor 5, exopolyphosphatase, Fe—S subunit of oxidoreductase, flavodoxin, fumarylacetoacetate hydrolase, gamma-glutamyltranspeptidase, GDP-mannose dehydrogenase, geranylgeranyl pyrophosphate synthase, gibberellin 20-oxidase, Glu/Leu/Phe/Val dehydrogenase , glucokinase, glucose dehydrogenase, glucose-1-phosphate cytidylyltransferase, glucose-6-phosphate 1-dehydrogenase, glutamate ammonia-ligase, glutamine amidotransferase, glutamine synthetase, glutaredoxin, glutathione peroxidase, glutathione S-transferase, Glycine cleavage system T aminomethyltransferase, glycogen (starch) synthase, glycogen synthase, glycoprotease, glycosyl transferase, GM02LC11114-protein, GM02LC17485-protein, harpin-induced family protein, heat shock transcription factor, HesB/YadR/YfhF family protein, histone acetyltransferase, histone H2A, homocitrate synthase, homoserine O-acetyltransferase, hydrolase, integral membrane transporter family protein, iron(III) dicitrate-binding protein, isochorismate synthase, kinase, L-aspartate oxidase, lipoprotein precursor, lysine decarboxylase, lysyl-tRNA synthetase, major facilitator superfamily transporter protein, malate dehydrogenase, malic enzyme, membrane protein, membrane transport protein, metal ion transporter, metal-dependent hydrolase, methylglyoxal synthase, methylglyoxalase, methyltransferase, mitochondrial inner membrane protease subunit, molecular chaperone portein, monothiol glutaredoxin, monthiol glutaredoxin, multidrug resistance protein, multiple antibiotic resistance protein, murein transglycosylase, N-(5′-phosphoribosyl)anthranilate isomerase, Na+/K+ transporter, N-acetyl-gamma-glutamyl-phosphate reductase, NADH dehydrogenase I chain I, NADH dehydrogenase subunit N, NADH-quinone oxidoreductase subunit, NADP-dependent malic enzyme, nitrate/nitrite transport protein, O-antigen chain length determinant, oxidoreductase, oxidoreductase subunit, oxireductase, peptidase, permease protein of phosphate ABC transporter, phosphate permease, phosphate transporter, phosphoadenosine phosphosulfate reductase , phosphoanhydride phosphorylase, phosphoenolpyruvate carboxykinase, phosphopantetheine adenylyltransferase, phosphoribosylformyl glycinamidine synthase subunit, Photosystem I reaction center subunit XI, photosystem II protein, polygalacturonase, precorrin methylase, Pre-mRNA-splicing factor, prolipoprotein diacylglyceryl transferase, protease, protein kinase, protein phosphatase, PTS system N-acetylmuramic acid-specific EIIBC component, purine biosynthesis protein, pyruvate kinase, restriction alleviation protein, riboflavin biosynthesis protein, RNA binding protein, RNA polymerase sigma factor, RNA-binding protein, s_pp015018333r-protein, sec-independent protein translocase, Sec-independent protein translocase subunit, serine acetyltransferase, serine hydroxymethyltransferase, serine protease, serine/threonine dehydratase, Serine/threonine-protein phosphatase, short-chain alcohol dehydrogenase family, sll0281-protein, sll1032-protein, sll1761-protein, sn-glycerol-3-phosphate dehydrogenase, sodium/proton antiporter, sterol 24-C-methyltransferase, thioredoxin, thioredoxin family protein, threonine dehydratase, threonine synthase, transcription factor, transcriptional regulator, transport protein, trehalose-phosphatase, tryptophan biosynthesis protein, tryptophan synthase alpha chain, TTC0768-protein, TTC0881protein, TTC1386-protein, urease subunit, uridine/cytidine kinase, uroporphyrin-111 C-methyltransferase, XM_{—}473199-protein, ydr183w-protein, ydr273w-protein, ydr507cprotein, yer014w-protein, yer106w-protein, YFL019C-protein, yfl054c-protein, ygl096w-protein, ygl237c-protein, ygr221 c-protein, yhl013c-protein, yhr006w-protein, yhr207c-protein, YKL038W-protein, yml083c-protein, ymr013c-protein, YNL086W-protein, yol160w-protein, zinc finger protein, zinc transporter, or Zm_{—}4842_BE510522-protein, in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4-dihydroxyphenylalanine (dopa), 5-oxoproline, alanine, asparagine, aspartate, citrulline, glycine, homoserine, ornithine, phenylalanine, serine or tyrosine, respectively, or a composition comprising 3,4-dihydroxyphenylalanine (dopa), 5-oxoproline, alanine, asparagine, aspartate, citrulline, glycine, homoserine, ornithine, phenylalanine, serine or tyrosine, respectively, in said non-human organism or in the culture medium surrounding said non-human organism.

Item 2. A process for the production of a respective fine chemical 3,4-dihydroxyphenylalanine (dopa), 5-oxoproline, alanine, asparagine, aspartate, citrulline, glycine, homoserine, ornithine, phenylalanine, serine or tyrosine, which comprises
(A)
(i) increasing or generating of the expression of; and/or
(ii) increasing or generating the expression of an expression product of; and/or
(iii) increasing or generating one or more activities of an expression product encoded by; at least one respective nucleic acid molecule comprising a nucleic acid molecule selected from the group consisting of:
(a) a nucleic acid molecule encoding the polypeptide shown in column 5 or 8 of Table II, preferably Table II B, application no. 3, or a homolog or a fragment thereof, whereby the respective line discloses in column 7 3,4-dihydroxyphenylalanine (dopa), 5-oxoproline, alanine, asparagine, aspartate, citrulline, glycine, homoserine, ornithine, phenylalanine, serine or tyrosine, respectively;
(b) a nucleic acid molecule shown in column 5 or 8 of Table I, preferably Table I B, application no. 3, or a homolog or a fragment thereof (preferably the coding region thereof) , whereby the respective line discloses in column 7 3,4-dihydroxyphenylalanine (dopa), 5-oxoproline, alanine, asparagine, aspartate, citrulline, glycine, homoserine, ornithine, phenylalanine, serine or tyrosine, respectively;
(c) a nucleic acid molecule, which, as a result of the degeneracy of the genetic code, can be derived from a polypeptide sequence depicted in column 5 or 8 of Table II, preferably Table II B, application no. 3, whereby the respective line discloses in column 7 3,4-dihydroxyphenylalanine (dopa), 5-oxoproline, alanine, asparagine, aspartate, citrulline, glycine, homoserine, ornithine, phenylalanine, serine or tyrosine, respectively; (d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 3, or the coding region thereof, whereby the respective line discloses in column 7 3,4-dihydroxyphenylalanine (dopa), 5-oxoproline, alanine, asparagine, aspartate, citrulline, glycine, homoserine, ornithine, phenylalanine, serine or tyrosine, respectively;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 3, whereby the respective line discloses in column 7 3,4-dihydroxyphenylalanine (dopa), 5-oxoproline, alanine, asparagine, aspartate, citrulline, glycine, homoserine, ornithine, phenylalanine, serine or tyrosine, respectively;
(i) a nucleic acid molecule encoding a polypeptide having the activity represented by a respective protein as depicted in column 5 of Table II, application no. 3, the respective line discloses in column 7 3,4-dihydroxyphenylalanine (dopa), 5-oxoproline, alanine, asparagine, aspartate, citrulline, glycine, homoserine, ornithine, phenylalanine, serine or tyrosine, respectively ;
(j) a nucleic acid molecule which comprises a polynucleotide, which is obtained by amplifying a cDNA library or a genomic library using the primers as depicted in column 8 of Table III, application no. 3, whereby the respective line discloses in column 7 3,4-dihydroxyphenylalanine (dopa), 5-oxoproline, alanine, asparagine, aspartate, citrulline, glycine, homoserine, ornithine, phenylalanine, serine or tyrosine, respectively; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
   or a nucleic acid molecule comprising a sequence which is complementary thereto;
   in a non-human organism or a part thereof, preferably a microorganism, a plant cell, a plant or a part thereof, as compared to a corresponding non-transformed wild type non-human organism or a part thereof; and
(B) growing the non-human organism or a part thereof under conditions which permit the production of 3,4-dihydroxyphenylalanine (dopa), 5-oxoproline, alanine, asparagine, aspartate, citrulline, glycine, homoserine, ornithine, phenylalanine, serine or tyrosine, respectively or a composition comprising 3,4-dihydroxyphenylalanine (dopa), 5-oxoproline, alanine, asparagine, aspartate, citrulline, glycine, homoserine, ornithine, phenylalanine, serine or tyrosine, respectively, in said non-human organism or in the culture medium surrounding said non-human organism.

Item 3. A process of items 1 or 2, comprising of recovering 3,4-dihydroxyphenylalanine (dopa), 5-oxoproline, alanine, asparagine, aspartate, citrulline, glycine, homoserine, ornithine, phenylalanine, serine or tyrosine, respectively, in its free or bound form.

Item 4. The process of any one of items 1 to 3, comprising the folowing steps:
(a) selecting a non-human organism or a part thereof expressing a polypeptide encoded by the nucleic acid molecule characterized in item 2;
(b) mutagenizing the selected non-human organism or the part thereof;
(c) comparing the activity or the expression level of said polypeptide in the mutagenized non-human organism or the part thereof with the activity or the expression of said polypeptide of the selected non-human organisms or the part thereof;
(d) selecting the mutated non-human organisms or parts thereof, which comprise an increased activity or expression level of said polypeptide compared to the selected non-human organism or the part thereof;
(e) optionally, growing and cultivating the non-human organisms or the parts thereof;
and
(f) recovering, and optionally isolating, the free or bound 3,4-dihydroxyphenylalanine (dopa), 5-oxoproline, alanine, asparagine, aspartate, citrulline, glycine, homoserine, ornithine, phenylalanine, serine or tyrosine, respectively, produced by the selected mutated non-human organisms or parts thereof.

Item 5. The process of any one of items 1 to 4 wherein the activity of said protein or the expression of said nucleic acid molecule is increased or generated transiently or stably.

Item 6. An isolated nucleic acid molecule comprising a nucleic acid molecule selected from the group consisting of:
(a) a nucleic acid molecule encoding a polypeptide shown in Table II, application no. 3, column 5 or 8, preferably shown in Table II B, application no. 3, column 8, or a homolog or a fragment thereof;
(b) a nucleic acid molecule shown in Table I, application no. 3, column 5 or 8, preferably shown in Table I B, application no. 3, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 3, preferably in column 8 of Table II B, application no. 3;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 3, preferably shown in column 8 of Table I B, application no. 3, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 3;
(i) a nucleic acid molecule encoding a polypeptide having the activity represented by a respective protein as depicted in column 5 of Table II, application no. 3;
(j) a nucleic acid molecule comprising a polynucleotide which is obtained by amplifying a cDNA library or a genomic library using the primers in Table III, application no. 3, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto.

Item 7. A nucleic acid construct which confers the expression of the nucleic acid molecule as defined in item 6, comprising one or more regulatory elements.

Item 8. An expression cassette comprising
(a) a promoter, preferably selected from the group consisting of Big35S, PCUbi, Super ans USP;
(b) a nucleic acid molecule as defined in item 6.

Item 9. A vector comprising the nucleic acid molecule as defined in item 6 or the nucleic acid construct as claimed in item 7 or the expression cassette as claimed in item 8.

Item 10. A host cell or a plant organelle, which has been transformed with the vector as claimed in item 9 or the nucleic acid molecule as defined in item 6 or the nucleic acid construct as claimed in item 7 or the expression cassette as claimed in item 8.

Item 11. The host cell of item 10, which is a plant cell or a microorganism.

Item 12. A process for producing a polypeptide, wherein the polypeptide is expressed in a host cell as claimed in item 10 or 11.

Item 13. A polypeptide produced by the process as claimed in item 12 or encoded by the nucleic acid molecule as defined in item 6.

Item 14. An antibody, which binds specifically to the polypeptide as claimed in item 13.

Item 15. A plant tissue, propagation material, harvested material or a plant or a part thereof comprising the host cell as claimed in item 10 or 11.

Item 16. A process for the identification of a compound conferring an increase in 3,4-dihydroxyphenylalanine (dopa), 5-oxoproline, alanine, asparagine, aspartate, citrulline, glycine, homoserine, ornithine, phenylalanine, serine or tyrosineproduction, respectively, in a non-human organism, comprising the steps:
(a) culturing a plant cell or tissue or microorganism or maintaining a plant expressing the polypeptide encoded by the nucleic acid molecule as defined in item 6 conferring an increase in the amount of 3,4-dihydroxyphenylalanine (dopa), 5-oxoproline, alanine, asparagine, aspartate, citrulline, glycine, homoserine, ornithine, phenylalanine, serine or tyrosine, respectively, in a non-human organism or a part thereof and a readout system capable of interacting with the polypeptide under suitable conditions which permit the interaction of the polypeptide with said readout system in the presence of a compound or a sample comprising a plurality of compounds and capable of providing a detectable signal in response to the binding of a compound to said polypeptide under conditions which permit the expression of said readout system and of the polypeptide encoded by the nucleic acid molecule of item 6 conferring an increase in the amount of 3,4-dihydroxyphenylalanine (dopa), 5-oxoproline, alanine, asparagine, aspartate, citrulline, glycine, homoserine, ornithine, phenylalanine, serine or tyrosine, respectively, in a non-human organism or a part thereof;
(b) identifying if the compound is an effective agonist by detecting the presence or absence or increase of a signal produced by said readout system.

Item 17. A method for the production of an agricultural composition comprising the steps of the method of item 16 and formulating the compound identified in item 16 in a form acceptable for an application in agriculture.

Item 18. A composition comprising the nucleic acid molecule as defined in item 6, the polypeptide as claimed in item 13, the nucleic acid construct as claimed in item 7, the expression cassette as claimed in item 8, the vector as claimed in item 9, the compound as claimed in item 16, the antibody as claimed in item 14, and optionally an agricultural acceptable carrier.

Item 19. Use of the nucleic acid molecule as defined in item 6 for the identification of a nucleic acid molecule conferring an increase in 3,4-dihydroxyphenylalanine (dopa), 5-oxoproline, alanine, asparagine, aspartate, citrulline, glycine, homoserine, ornithine, phenylalanine, serine or tyrosine, respectively, after expression.

Item 20. Cosmetic, pharmaceutical, food or feed composition comprising the nucleic acid moleculeas defined in item 6, the polypeptide as claimed in item 13, the nucleic acid construct as claimed in item 7, the expression cassette as claimed in item 8, the vector as claimed in item 9, the antibody as claimed in item 14, the plant or a part thereof, or plant tissue as claimed in item 15, the harvested material or the propagation material as claimed in item 15 or the host cell as claimed in items 10 or 11.

Item 21. Use of the nucleic acid molecule as defined in item 6, the polypeptide as claimed in item 13, the nucleic acid construct as claimed in item 7, the expression cassette as claimed in item 8, the vector as claimed in item 9, the plant or plant tissue as claimed in item 15, or the host cell as claimed in item 10 to 11 for the production of plant resistant to a herbicide inhibiting the production of 3,4-dihydroxyphenylalanine (dopa), 5-oxoproline, alanine, asparagine, aspartate, citrulline, glycine, homoserine, ornithine, phenylalanine, serine or tyrosine, respectively.

Item 22. A method for producing a plant with increased yield as compared to a corresponding non-transformed wild type plant, whereby the method comprises
(i) increasing or generating of the expression of; and/or
(ii) increasing or generating the expression of an expression product of; and/or
(iii) increasing or generating one or more activities of an expression product encoded by;
at least one respective nucleic acid molecule comprising a nucleic acid molecule selected from the group consisting of:
(a) a nucleic acid molecule encoding the polypeptide shown in column 5 or 8 of Table II, preferably Table II B, application no. 3, or a homolog or a fragment thereof, in case in column 7 aspartate, asparagine, alanine, citrulline or ornithine, respectively, is indicated;
(b) a nucleic acid molecule shown in column 5 or 8 of Table I, preferably Table I B, application no. 3, or a homolog or a fragment thereof (preferably the coding region thereof) , in case in column 7 aspartate, asparagine, alanine, citrulline or ornithine, respectively is indicated;
(c) a nucleic acid molecule, which, as a result of the degeneracy of the genetic code, can be derived from a polypeptide sequence depicted in column 5 or 8 of Table II, preferably Table II B, application no. 3, in case in column 7 aspartate, asparagine, alanine, citrulline or ornithine, respectively, is indicated;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 3, or the coding region thereof, in case in column 7 aspartate, asparagine, alanine, citrulline or ornithine, respectively, is indicated;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 3, in case in column 7 aspartate, asparagine, alanine, citrulline or ornithine, respectively, is indicated;
(i) a nucleic acid molecule encoding a polypeptide having the activity represented by a respective protein as depicted in column 5 of Table II, application no. 3, in case in column 7 aspartate, asparagine, alanine, citrulline or ornithine, respectively, is indicated;
(j) a nucleic acid molecule which comprises a polynucleotide, which is obtained by amplifying a cDNA library or a genomic library using the primers as depicted in column 8 of Table III, application no. 3, in case in column 7 aspartate, asparagine, alanine, citrulline or ornithine, respectively, is indicated; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j); or a nucleic acid molecule comprising a sequence which is complementary thereto; in a plant cell, plant or a part thereof.

Item 23. A method for producing a plant with increased yield according to item 22, which comprises in addition the regeneration of a plant from such transformed plant cell, plant cell compartment, plant cell nucleus, plant cell or plant tissue with increased yield.

[0483.1.1.3] to [0494.1.1.3] for the disclosure of these paragraphs see [0483.1.1.1] to [0494.1.1.1] above.

### Example 11a

### PCR Amplification of the Sequences

Unless otherwise specified, standard methods as described in Sambrook et al., Molecular Cloning: A laboratory manual, Cold Spring Harbor 1989, Cold Spring Harbor Laboratory Press are used.

The inventive sequences as shown in the respective line in Table I, column 5, preferably the coding region thereof, were amplified by PCR as described in the protocol of the Pfu Ultra, Pfu Turbo or Herculase DNA polymerase (Stratagene). The composition for the protocol of the Pfu Ultra, Pfu Turbo or Herculase DNA polymerase was as follows: 1×PCR buffer (Stratagene), 0.2 mM of each dNTP, 100 ng genomic DNA of Saccharomyces cerevisiae (strain S288C; Research Genetics, Inc., now Invitrogen), Escherichia coli (strain MG1655; E. coli Genetic Stock Center), Synechocystis sp. (strain PCC6803), Azotobacter vinelandii (strain N. R. Smith, 16), Thermus thermophilus (HB8) or 50 ng cDNA from various tissues and development stages of Arabidopsis thaliana (ecotype Columbia), Physcomitrella patens, Glycine max (variety Resnick), Brassica napus, Oryza sativa or Zea mays (variety B73, Mo17, A188), 50 pmol forward primer, 50 pmol reverse primer, with or without 1 M Betaine, 2.5 u Pfu Ultra, Pfu Turbo or Herculase DNA polymerase.

The amplification cycles were as follows: 1 cycle of 2-3 minutes at 94-95° C., then 25-36 cycles with 30-60 seconds at 94-95° C., 30-45 seconds at 50-60° C. and 210-480 seconds at 72° C., followed by 1 cycle of 5-10 minutes at 72° C., then 4-16° C.—preferably for Saccharomyces cerevisiae, Escherichia coli, Synechocystis sp., Azotobacter vinelandii, Thermus thermophilus.

In case of Arabidopsis thaliana, Brassica napus, Glycine max, Oryza sativa, Physcomitrella patens, Zea mays the amplification cycles were as follows:

1 cycle with 30 seconds at 94° C., 30 seconds at 61° C., 15 minutes at 72° C., then 2 cycles with 30 seconds at 94° C., 30 seconds at 60° C., 15 minutes at 72° C., then 3 cycles with 30 seconds at 94° C., 30 seconds at 59° C., 15 minutes at 72° C., then 4 cycles with 30 seconds at 94° C., 30 seconds at 58° C., 15 minutes at 72° C., then 25 cycles with 30 seconds at 94° C., 30 seconds at 57° C., 15 minutes at 72° C., then 1 cycle with 10 minutes at 72° C., then finally 4-16° C.

RNAs were generated with the RNeasy Plant Kit according to the standard protocol (Qiagen) and Superscript II Reverse Transkriptase was used to produce double stranded cDNA according to the standard protocol (Invitrogen).

ORF specific primer pairs for the genes to be expressed are shown in the respective line in Table III, column 8. The following adapter sequences were added to Saccharomyces cerevisiae ORF specific primers (see Table III) for cloning purposes:
i) foward primer: 5′-GGAATTCCAGCTGACCACC-3″ SEQ ID NO: 20
ii) reverse primer: 5′-GATCCCCGGGAATTGCCATG-3″ SEQ ID NO: 21

These adaptor sequences allow cloning of the ORF into the various vectors containing the Resgen adaptors, see table column E of Table c.

The following adapter sequences were added to Saccharomyces cerevisiae, Escherichia coli, Synechocystis sp., Azotobacter vinelandii, Thermus thermophilus, Arabidopsis thaliana, Brassica napus, Glycine max, Oryza sativa , Physcomitrella patens, or Zea mays ORF specific primers for cloning purposes:
iii) forward primer: 5″-TTGCTCTTCC-3′ SEQ ID NO: 22

iiii) reverse primer: 5′-TTGCTCTTCG-3′ SEQ ID NO: 23

The adaptor sequences allow cloning of the ORF into the various vectors containing the Colic adaptors, see table column E of Table c.

Therefore for amplification and cloning of Saccharomyces cerevisiae SEQ ID NO: 14843, a primer consisting of the adaptor sequence i) and the ORF specific sequence SEQ ID NO: 14871 and a second primer consisting of the adaptor sequence ii) and the ORF specific sequence SEQ ID NO: 14872 were used.

For amplification and cloning of Escherichia coli SEQ ID NO: 6818, a primer consisting of the adaptor sequence iii) and the ORF specific sequence SEQ ID NO: 7076 and a second primer consisting of the adaptor sequence iiii) and the ORF specific sequence SEQ ID NO: 7077 were used.

For amplification and cloning of Synechocystis sp. SEQ ID NO: 11211, a primer consisting of the adaptor sequence iii) and the ORF specific sequence SEQ ID NO: 11419 and a second primer consisting of the adaptor sequence iiii) and the ORF specific sequence SEQ ID NO: 11420 were used.

For amplification and cloning of Arabidopsis thaliana SEQ ID NO: 385, a primer consisting of the adaptor sequence iii) and the ORF specific sequence SEQ ID NO: 623 and a second primer consisting of the adaptor sequence iiii) and the ORF specific sequence SEQ ID NO: 624 were used.

For amplification and cloning of Azotobacter vinelandii SEQ ID NO: 5557, a primer consisting of the adaptor sequence iii) and the ORF specific sequence SEQ ID NO: 6033 and a second primer consisting of the adaptor sequence iiii) and the ORF specific sequence SEQ ID NO: 6034 were used.

For amplification and cloning of Brassica napus SEQ ID NO: 15532, a primer consisting of the adaptor sequence iii) and the ORF specific sequence SEQ ID NO: 16150 and a second primer consisting of the adaptor sequence iiii) and the ORF specific sequence SEQ ID NO: 16151 were used.

For amplification and cloning of Glycine max SEQ ID NO: 69, a primer consisting of the adaptor sequence iii) and the ORF specific sequence SEQ ID NO: 343 and a second primer consisting of the adaptor sequence iiii) and the ORF specific sequence SEQ ID NO: 344 were used.

For amplification and cloning of Oryza sativa SEQ ID NO: 34301, a primer consisting of the adaptor sequence iii) and the ORF specific sequence SEQ ID NO: 34595 and a second primer consisting of the adaptor sequence iiii) and the ORF specific sequence SEQ ID NO: 34596 were used.

For amplification and cloning of Physcomytrella patens SEQ ID NO: 45757, a primer consisting of the adaptor sequence iii) and the ORF specific sequence SEQ ID NO: 45791 and a second primer consisting of the adaptor sequence iiii) and the ORF specific sequence SEQ ID NO: 45792 were used.

For amplification and cloning of Thermus thermophilus SEQ ID NO: 12698, a primer consisting of the adaptor sequence iii) and the ORF specific sequence SEQ ID NO: 12970 and a second primer consisting of the adaptor sequence iiii) and the ORF specific sequence SEQ ID NO: 12971 were used.

For amplification and cloning of Zea mays SEQ ID NO: 68413, a primer consisting of the adapfor sequence iii) and the ORF specific sequence SEQ ID NO: 68531 and a second primer consisting of the adaptor sequence iiii) and the ORF specific sequence SEQ ID NO: 68532 were used.

Following these examples every sequence disclosed in Table I, preferably column 5, especially the coding region thereof can be cloned by fusing the adaptor sequences to the respective specific primers sequences as disclosed in Table III, column 8 using the respective vectors shown in Table c.

[0496.1.1.3] to [0499.1.1.3] for the disclosure of these paragraph see [0496.1.1.1] to [0499.1.1.1] above.

### Example 11e

### Cloning of Inventive Sequences as Shown in Table I, Column 5 in the Different Expression Vectors

For cloning of for example SEQ ID NO: 6818 from Escherichia coli or any other ORF from Escherichia coli, Synechocystis sp., Azotobacter vinelandii, Thermus thermophilus, Arabidopsis thaliana, Brassica napus, Glycine max, Oryza sativa , Physcomitrella patens, or Zea mays the vector DNA was treated with the restriction enzymes Pacl and Ncol following the standard protocol (MBI Fermentas).

For cloning for example the ORFs of SEQ ID NO: 14843 from Saccharomyces cerevisiae or any other ORF from Saccharomyces cerevisiae into vectors containing the Resgen adaptor sequence the respective vector DNA was treated with the restriction enzyme Ncol.

For cloning of for example ORFs SEQ ID NO: 63745 from Saccharomyces cerevisiae or any other ORF from Saccharomyces cerevisiae into vectors containing the Colic adaptor sequence, the respective vector DNA was treated with the restriction enzymes Pacl and Ncol following the standard protocol (MBI Fermentas).

In all cases the reaction was stopped by inactivation at 70° C. for 20 minutes and purified over QIAquick or NucleoSpin Extract II columns following the standard protocol (Qiagen or Macherey-Nagel).

Then the PCR-product representing the amplified ORF with the respective adapter sequences and the vector DNA were treated with T4 DNA polymerase according to the standard protocol (MBI Fermentas) to produce single stranded overhangs with the parameters 1 unit T4 DNA polymerase at 37° C. for 2-10 minutes for the vector and 1-2 u T4 DNA polymerase at 15-17° C. for 10-60 minutes for the PCR product representing SEQ ID NO: 6818.

The reaction was stopped by addition of high-salt buffer and purified over QIAquick or NucleoSpin Extract II columns following the standard protocol (Qiagen or Macherey-Nagel).

According to this example the skilled person is able to clone all sequences disclosed in Table I, preferably column 5 or column 8, especially the coding region thereof.

Approximately 30-60 ng of prepared vector and a defined amount of prepared amplificate were mixed and hybridized at 65° C. for 15 minutes followed by 37° C. 0,1 ° C./1 seconds, followed by 37° C. 10 minutes, followed by 0,1 ° C./1 seconds, then 4-10 ° C.

The ligated constructs were transformed in the same reaction vessel by addition of competent E. coli cells (strain DH5alpha) and incubation for 20 minutes at 1° C. followed by a heat shock for 90 seconds at 42° C. and cooling to 1-4° C. Then, complete medium (SOC) was added and the mixture was incubated for 45 minutes at 37° C. The entire mixture was subsequently plated onto an agar plate with 0.05 mg/ml kanamycin and incubated overnight at 37° C.

The outcome of the cloning step was verified by amplification with the aid of primers which bind upstream and downstream of the integration site, thus allowing the amplification of the insertion. The amplifications were carried out as described in the protocol of Taq DNA polymerase (Gibco-BRL).

The amplification cycles were as follows:

1 cycle of 1-5 minutes at 94° C., followed by 35 cycles of in each case 15-60 seconds at 94° C., 15-60 seconds at 50-66° C. and 5-15 minutes at 72° C., followed by 1 cycle of 10 minutes at 72° C., then 4-16° C.

Several colonies were checked, but only one colony for which a PCR product of the expected size was detected was used in the following steps.

A portion of this positive colony was transferred into a reaction vessel filled with complete medium (LB) supplemented with kanamycin and incubated overnight at 37° C.

The plasmid preparation was carried out as specified in the Qiaprep or NucleoSpin Multi-96 Plus standard protocol (Qiagen or Macherey-Nagel).

[0501.1.1.3] to [0503.1.1.3] for the disclosure of these paragraphs see [0501.1.1.1] to [0503.1.1.1] above. Table d showing results of plant analyses

TABLE d

| SeqID | Target | Locus | Metabolite | Source | Promotor | Method | Min | Max |
|---|---|---|---|---|---|---|---|---|
| 15187 | non- | 47266012_SOYBEAN | aspartate | ARA_LEAF | p-PcUBI | GC | 48 | 125 |
| | targeted | | | | | | | |
| 15187 | non- | 47266012_SOYBEAN | tyrosine | ARA_LEAF | p-PcUBI | GC | 44 | 122 |
| | targeted | | | | | | | |
| 15187 | non- | 47266012_SOYBEAN | 5-oxo- | ARA_LEAF | p-PcUBI | GC | 52 | 161 |
| | targeted | | proline | | | | | |
| 69 | non- | 49747384_SOYBEAN | aspartate | ARA_LEAF | p-PcUBI | GC | 55 | 122 |
| | targeted | | | | | | | |
| 69 | non- | 49747384_SOYBEAN | tyrosine | ARA_LEAF | p-PcUBI | GC | 35 | 80 |
| | targeted | | | | | | | |
| 69 | non- | 49747384_SOYBEAN | 5-oxo- | ARA_LEAF | p-PcUBI | GC | 52 | 101 |
| | targeted | | proline | | | | | |
| 15532 | plastidic | 51340801_CANOLA | alanine | ARA_LEAF | p-PcUBI | GC | 38 | 153 |
| 15532 | plastidic | 51340801_CANOLA | phenyl- | ARA_LEAF | p-PcUBI | GC | 30 | 263 |
| | | | alanine | | | | | |
| 15532 | plastidic | 51340801_CANOLA | tyrosine | ARA_LEAF | p-PcUBI | LC | 42 | 123 |
| 16155 | non- | 59547452_SOYBEAN | citrulline | ARA_LEAF | p-PcUBI | LC | 40 | 104 |
| | targeted | | | | | | | |
| 16263 | plastidic | 59554615_SOYBEAN | asparagine | ARA_LEAF | p-PcUBI | GC | 54 | 113 |
| 16263 | plastidic | 59554615_SOYBEAN | serine | ARA_LEAF | p-PcUBI | GC | 27 | 31 |
| 16883 | plastidic | 59582753_SOYBEAN | alanine | ARA_LEAF | p-PcUBI | GC | 23 | 116 |
| 16883 | plastidic | 59582753_SOYBEAN | phenyl- | ARA_LEAF | p-PcUBI | GC | 27 | 61 |
| | | | alanine | | | | | |
| 16883 | plastidic | 59582753_SOYBEAN | tyrosine | ARA_LEAF | p-PcUBI | GC | 38 | 93 |
| 17356 | plastidic | AAC43185 | alanine | ARA_LEAF | p-PcUBI | GC | 22 | 31 |
| 17356 | plastidic | AAC43185 | citrulline | ARA_LEAF | p-PcUBI | LC | 76 | 618 |
| 17356 | plastidic | AAC43185 | phenyl- | ARA_LEAF | p-PcUBI | GC | 26 | 55 |
| | | | alanine | | | | | |
| 17451 | non- | At1g07430 | citrulline | ARA_LEAF | p-PcUBI | LC | 34 | 55 |
| | targeted | | | | | | | |
| 385 | non- | At1g14490 | glycine | ARA_LEAF | p-PcUBI | GC | 97 | 147 |
| | targeted | | | | | | | |
| 385 | non- | At1g14490 | serine | ARA_LEAF | p-PcUBI | GC | 27 | 51 |
| | targeted | | | | | | | |
| 17601 | non- | At1g17440 | phenyl- | ARA_LEAF | p-PcUBI | LC | 33 | 54 |
| | targeted | | alanine | | | | | |
| 17601 | non- | At1g17440 | tyrosine | ARA_LEAF | p-PcUBI | GC | 48 | 106 |
| | targeted | | | | | | | |
| 17637 | non- | At1g19800 | aspartate | ARA_LEAF | p-PcUBI | GC | 49 | 86 |
| | targeted | | | | | | | |
| 17637 | non- | At1g19800 | citrulline | ARA_LEAF | p-PcUBI | LC | 35 | 66 |
| | targeted | | | | | | | |
| 17637 | non- | At1g19800 | tyrosine | ARA_LEAF | p-PcUBI | LC | 41 | 398 |
| | targeted | | | | | | | |
| 17701 | non- | At1g26830 | phenyl- | ARA_LEAF | p-PcUBI | GC | 35 | 134 |
| | targeted | | alanine | | | | | |
| 17701 | non- | At1g26830 | tyrosine | ARA_LEAF | p-PcUBI | LC | 39 | 89 |
| | targeted | | | | | | | |
| 627 | non- | At1g31230 | homo- | ARA_LEAF | Big35S | GC | 84 | 1108 |
| | targeted | | serine | | | | | |
| 17968 | non- | At1g36730 | aspartate | ARA_LEAF | p-PcUBI | GC | 51 | 87 |
| | targeted | | | | | | | |
| 17968 | non- | At1g36730 | phenyl- | ARA_LEAF | p-PcUBI | GC | 32 | 97 |
| | targeted | | alanine | | | | | |
| 17968 | non- | At1g36730 | serine | ARA_LEAF | p-PcUBI | GC | 25 | 72 |
| | targeted | | | | | | | |
| 17968 | non- | At1g36730 | tyrosine | ARA_LEAF | p-PcUBI | GC | 36 | 86 |
| | targeted | | | | | | | |
| 17968 | non- | At1g36730 | 5-oxo- | ARA_LEAF | p-PcUBI | GC | 39 | 74 |
| | targeted | | proline | | | | | |
| 18070 | non- | At1g43850 | phenyl- | ARA_LEAF | p-PcUBI | GC | 77 | 358 |
| | targeted | | alanine | | | | | |
| 18070 | non- | At1g43850 | serine | ARA_LEAF | p-PcUBI | GC | 70 | 205 |
| | targeted | | | | | | | |
| 18070 | non- | At1g43850 | tyrosine | ARA_LEAF | p-PcUBI | GC | 76 | 313 |
| | targeted | | | | | | | |
| 18070 | non- | At1g43850 | 5-oxo- | ARA_LEAF | p-PcUBI | GC | 41 | 210 |
| | targeted | | proline | | | | | |
| 812 | non- | At1g48040 | glycine | ARA_LEAF | p-PcUBI | GC | 36 | 52 |
| | targeted | | | | | | | |
| 18235 | non- | At1g48260 | phenyl- | ARA_LEAF | p-PcUBI | GC | 58 | 432 |
| | targeted | | alanine | | | | | |
| 18235 | non- | At1g48260 | serine | ARA_LEAF | p-PcUBI | GC | 29 | 154 |
| | targeted | | | | | | | |
| 18235 | non- | At1g48260 | tyrosine | ARA_LEAF | p-PcUBI | LC | 48 | 182 |
| | targeted | | | | | | | |
| 18869 | non- | At1g61950 | aspartate | ARA_LEAF | p-PcUBI | GC | 60 | 312 |
| | targeted | | | | | | | |
| 18869 | non- | At1g61950 | phenyl- | ARA_LEAF | p-PcUBI | GC | 65 | 149 |
| | targeted | | alanine | | | | | |
| 19419 | non- | At1g72770 | alanine | ARA_LEAF | p-PcUBI | GC | 26 | 121 |
| | targeted | | | | | | | |
| 19419 | non- | At1g72770 | glycine | ARA_LEAF | p-PcUBI | GC | 48 | 108 |
| | targeted | | | | | | | |
| 19419 | non- | At1g72770 | tyrosine | ARA_LEAF | p-PcUBI | GC | 35 | 150 |
| | targeted | | | | | | | |
| 19419 | non- | At1g72770 | 5-oxo- | ARA_LEAF | p-PcUBI | GC | 45 | 171 |
| | targeted | | proline | | | | | |
| 68536 | non- | At2g13360 | alanine | ARA_LEAF | p-PcUBI | GC | 23 | 44 |
| | targeted | | | | | | | |
| 19502 | non- | At2g17560 | phenyl- | ARA_LEAF | p-PcUBI | GC | 32 | 315 |
| | targeted | | alanine | | | | | |
| 19502 | non- | At2g17560 | tyrosine | ARA_LEAF | p-PcUBI | GC | 37 | 182 |
| | targeted | | | | | | | |
| 1298 | non- | At2g25070 | aspartate | ARA_LEAF | p-PcUBI | GC | 74 | 147 |
| | targeted | | | | | | | |
| 1298 | non- | At2g25070 | 5-oxo- | ARA_LEAF | p-PcUBI | GC | 38 | 78 |
| | targeted | | proline | | | | | |
| 19874 | non- | At2g28890 | asparagine | ARA_LEAF | p-PcUBI | GC | 57 | 929 |
| | targeted | | | | | | | |
| 19874 | non- | At2g28890 | phenyl- | ARA_LEAF | p-PcUBI | GC | 36 | 520 |
| | targeted | | alanine | | | | | |
| 19874 | non- | At2g28890 | serine | ARA_LEAF | p-PcUBI | GC | 29 | 164 |
| | targeted | | | | | | | |
| 19874 | non- | At2g28890 | tyrosine | ARA_LEAF | p-PcUBI | GC | 62 | 555 |
| | targeted | | | | | | | |
| 19919 | non- | At2g30360 | phenyl- | ARA_LEAF | p-PcUBI | GC | 29 | 548 |
| | targeted | | alanine | | | | | |
| 19919 | non- | At2g30360 | tyrosine | ARA_LEAF | p-PcUBI | GC | 38 | 323 |
| | targeted | | | | | | | |
| 19919 | non- | At2g30360 | 5-oxo- | ARA_LEAF | p-PcUBI | GC | 33 | 92 |
| | targeted | | proline | | | | | |
| 20346 | non- | At2g30540 | serine | ARA_LEAF | p-PcUBI | GC | 24 | 171 |
| | targeted | | | | | | | |
| 20346 | non- | At2g30540 | tyrosine | ARA_LEAF | p-PcUBI | LC | 43 | 106 |
| | targeted | | | | | | | |
| 20578 | non- | At2g34180 | glycine | ARA_LEAF | p-PcUBI | GC | 56 | 191 |
| | targeted | | | | | | | |
| 21008 | non- | At2g39800 | alanine | ARA_LEAF | p-PcUBI | GC | 20 | 34 |
| | targeted | | | | | | | |
| 21008 | non- | At2g39800 | glycine | ARA_LEAF | p-PcUBI | GC | 34 | 83 |
| | targeted | | | | | | | |
| 21159 | non- | At2g47880 | phenyl- | ARA_LEAF | p-PcUBI | LC | 64 | 241 |
| | targeted | | alanine | | | | | |
| 21159 | non- | At2g47880 | tyrosine | ARA_LEAF | p-PcUBI | LC | 62 | 219 |
| | targeted | | | | | | | |
| 68657 | non- | At3g02990 | tyrosine | ARA_LEAF | p-PcUBI | LC | 46 | 84 |
| | targeted | | | | | | | |
| 21497 | plastidic | At3g04050 | alanine | ARA_LEAF | p-PcUBI | GC | 30 | 950 |
| 21497 | plastidic | At3g04050 | phenyl- | ARA_LEAF | p-PcUBI | GC | 104 | 300 |
| | | | alanine | | | | | |
| 21497 | plastidic | At3g04050 | tyrosine | ARA_LEAF | p-PcUBI | GC | 41 | 346 |
| 21902 | non- | At3g04710 | phenyl- | ARA_LEAF | p-PcUBI | GC | 50 | 58 |
| | targeted | | alanine | | | | | |
| 22015 | non- | At3g06270 | tyrosine | ARA_LEAF | p-PcUBI | GC | 44 | 46 |
| | targeted | | | | | | | |
| 22249 | non- | At3g08710 | aspartate | ARA_LEAF | p-PcUBI | GC | 77 | 149 |
| | targeted | | | | | | | |
| 22249 | non- | At3g08710 | phenyl- | ARA_LEAF | p-PcUBI | GC | 57 | 205 |
| | targeted | | alanine | | | | | |
| 22249 | non- | At3g08710 | tyrosine | ARA_LEAF | p-PcUBI | GC | 50 | 132 |
| | targeted | | | | | | | |
| 22611 | non- | At3g11650 | phenyl- | ARA_LEAF | p-PcUBI | GC | 34 | 149 |
| | targeted | | alanine | | | | | |
| 22611 | non- | At3g11650 | serine | ARA_LEAF | p-PcUBI | GC | 24 | 89 |
| | targeted | | | | | | | |
| 22611 | non- | At3g11650 | tyrosine | ARA_LEAF | p-PcUBI | GC | 35 | 125 |
| | targeted | | | | | | | |
| 22832 | non- | At3g18524 | phenyl- | ARA_LEAF | p-PcUBI | GC | 30 | 48 |
| | targeted | | alanine | | | | | |
| 1815 | non- | At3g23000 | phenyl- | ARA_LEAF | p-PcUBI | GC | 75 | 455 |
| | targeted | | alanine | | | | | |
| 1815 | non- | At3g23000 | serine | ARA_LEAF | p-PcUBI | GC | 23 | 78 |
| | targeted | | | | | | | |
| 1815 | non- | At3g23000 | tyrosine | ARA_LEAF | p-PcUBI | GC | 67 | 183 |
| | targeted | | | | | | | |
| 1815 | non- | At3g23000 | 5-oxo- | ARA_LEAF | p-PcUBI | GC | 42 | 110 |
| | targeted | | proline | | | | | |
| 68727 | non- | At3g26400 | serine | ARA_LEAF | p-PcUBI | GC | 23 | 62 |
| | targeted | | | | | | | |
| 23002 | non- | At3g27300 | tyrosine | ARA_LEAF | p-PcUBI | GC | 37 | 87 |
| | targeted | | | | | | | |
| 68777 | non- | At3g27540 | phenyl- | ARA_LEAF | p-PcUBI | GC | 36 | 179 |
| | targeted | | alanine | | | | | |
| 68777 | non- | At3g27540 | tyrosine | ARA_LEAF | p-PcUBI | GC | 42 | 324 |
| | targeted | | | | | | | |
| 2367 | non- | At3g61830 | phenyl- | ARA_LEAF | p-PcUBI | GC | 58 | 540 |
| | targeted | | alanine | | | | | |
| 2367 | non- | At3g61830 | tyrosine | ARA_LEAF | p-PcUBI | GC | 36 | 784 |
| | targeted | | | | | | | |
| 2573 | non- | At3g62930 | aspartate | ARA_LEAF | p-PcUBI | GC | 48 | 117 |
| | targeted | | | | | | | |
| 2573 | non- | At3g62930 | phenyl- | ARA_LEAF | p-PcUBI | GC | 26 | 274 |
| | targeted | | alanine | | | | | |
| 2935 | non- | At3g62950 | phenylalanine | ARA_LEAF | p-PcUBI | GC | 105 | 324 |
| | targeted | | | | | | | |
| 2935 | non- | At3g62950 | tyrosine | ARA_LEAF | p-PcUBI | GC | 55 | 249 |
| | targeted | | | | | | | |
| 2935 | non- | At3g62950 | 5-oxo- | ARA_LEAF | p-PcUBI | GC | 37 | 235 |
| | targeted | | proline | | | | | |
| 23482 | non- | At4g15660 | phenyl- | ARA_LEAF | p-PcUBI | LC | 33 | 139 |
| | targeted | | alanine | | | | | |
| 23482 | non- | At4g15660 | tyrosine | ARA_LEAF | p-PcUBI | GC | 36 | 211 |
| | targeted | | | | | | | |
| 3279 | non- | At4g15670 | tyrosine | ARA_LEAF | p-PcUBI | GC | 58 | 140 |
| | targeted | | | | | | | |
| 23844 | non- | At4g15690 | aspartate | ARA_LEAF | p-PcUBI | GC | 75 | 169 |
| | targeted | | | | | | | |
| 23844 | non- | At4g15690 | phenyl- | ARA_LEAF | p-PcUBI | GC | 29 | 90 |
| | targeted | | alanine | | | | | |
| 23844 | non- | At4g15690 | tyrosine | ARA_LEAF | p-PcUBI | GC | 41 | 150 |
| | targeted | | | | | | | |
| 23844 | non- | At4g15690 | 5-oxo- | ARA_LEAF | p-PcUBI | GC | 36 | 106 |
| | targeted | | proline | | | | | |
| 3654 | non- | At4g15700 | tyrosine | ARA_LEAF | p-PcUBI | GC | 52 | 121 |
| | targeted | | | | | | | |
| 24232 | non- | At4g18880 | serine | ARA_LEAF | p-PcUBI | GC | 23 | 140 |
| | targeted | | | | | | | |
| 68849 | non- | At4g26080 | alanine | ARA_LEAF | p-PcUBI | GC | 24 | 81 |
| | targeted | | | | | | | |
| 68849 | non- | At4g26080 | aspartate | ARA_LEAF | p-PcUBI | GC | 54 | 123 |
| | targeted | | | | | | | |
| 68849 | non- | At4g26080 | citrulline | ARA_LEAF | p-PcUBI | LC | 42 | 59 |
| | targeted | | | | | | | |
| 4040 | non- | At4g32480 | alanine | ARA_LEAF | p-PcUBI | GC | 48 | 318 |
| | targeted | | | | | | | |
| 4040 | non- | At4g32480 | asparagine | ARA_LEAF | p-PcUBI | GC | 86 | 1206 |
| | targeted | | | | | | | |
| 4040 | non- | At4g32480 | glycine | ARA_LEAF | p-PcUBI | GC | 38 | 686 |
| | targeted | | | | | | | |
| 4040 | non- | At4g32480 | phenyl- | ARA_LEAF | p-PcUBI | GC | 31 | 410 |
| | targeted | | alanine | | | | | |
| 4040 | non- | At4g32480 | serine | ARA_LEAF | p-PcUBI | GC | 40 | 98 |
| | targeted | | | | | | | |
| 4040 | non- | At4g32480 | tyrosine | ARA_LEAF | p-PcUBI | GC | 54 | 437 |
| | targeted | | | | | | | |
| 4102 | non- | At4g33040 | aspartate | ARA_LEAF | p-PcUBI | GC | 48 | 194 |
| | targeted | | | | | | | |
| 4102 | non- | At4g33040 | citrulline | ARA_LEAF | p-PcUBI | LC | 30 | 52 |
| | targeted | | | | | | | |
| 24311 | non- | At4g34160 | phenyl- | ARA_LEAF | p-PcUBI | GC | 29 | 152 |
| | targeted | | alanine | | | | | |
| 4348 | non- | At4g35310 | asparagine | ARA_LEAF | p-PcUBI | GC | 52 | 443 |
| | targeted | | | | | | | |
| 4348 | non- | At4g35310 | phenylalanine | ARA_LEAF | p-PcUBI | GC | 191 | 534 |
| | targeted | | | | | | | |
| 4348 | non- | At4g35310 | serine | ARA_LEAF | p-PcUBI | GC | 67 | 140 |
| | targeted | | | | | | | |
| 4348 | non- | At4g35310 | tyrosine | ARA_LEAF | p-PcUBI | GC | 149 | 427 |
| | targeted | | | | | | | |
| 4348 | non- | At4g35310 | 5-oxo- | ARA_LEAF | p-PcUBI | GC | 99 | 175 |
| | targeted | | proline | | | | | |
| 68987 | non- | At5g02760 | tyrosine | ARA_LEAF | p-PcUBI | LC | 54 | 95 |
| | targeted | | | | | | | |
| 24438 | non- | At5g03720 | aspartate | ARA_LEAF | p-PcUBI | GC | 56 | 212 |
| | targeted | | | | | | | |
| 24438 | non- | At5g03720 | 3,4-di- | ARA_LEAF | p-PcUBI | GC | 48 | 124 |
| | targeted | | hydroxy | | | | | |
| | | | phenyl- | | | | | |
| | | | alanine | | | | | |
| | | | (dopa) | | | | | |
| 24492 | non- | At5g07200 | phenyl- | ARA_LEAF | p-PcUBI | GC | 30 | 100 |
| | targeted | | alanine | | | | | |
| 24492 | non- | At5g07200 | serine | ARA_LEAF | p-PcUBI | GC | 24 | 98 |
| | targeted | | | | | | | |
| 25222 | non- | At5g10820 | serine | ARA_LEAF | p-PcUBI | GC | 26 | 72 |
| | targeted | | | | | | | |
| 69275 | non- | At5g14070 | phenyl- | ARA_LEAF | p-PcUBI | GC | 27 | 127 |
| | targeted | | alanine | | | | | |
| 25283 | non- | At5g16650 | asparagine | ARA_LEAF | p-PcUBI | GC | 48 | 386 |
| | targeted | | | | | | | |
| 25283 | non- | At5g16650 | aspartate | ARA_LEAF | p-PcUBI | GC | 48 | 224 |
| | targeted | | | | | | | |
| 25283 | non- | At5g16650 | serine | ARA_LEAF | p-PcUBI | GC | 35 | 139 |
| | targeted | | | | | | | |
| 25283 | non- | At5g16650 | 5-oxo- | ARA_LEAF | p-PcUBI | GC | 45 | 158 |
| | targeted | | proline | | | | | |
| 4904 | non- | At5g18600 | phenyl- | ARA_LEAF | p-PcUBI | GC | 43 | 184 |
| | targeted | | alanine | | | | | |
| 4904 | non- | At5g18600 | tyrosine | ARA_LEAF | p-PcUBI | GC | 66 | 212 |
| | targeted | | | | | | | |
| 69553 | non- | At5g21910 | phenyl- | ARA_LEAF | p-PcUBI | GC | 29 | 67 |
| | targeted | | alanine | | | | | |
| 25428 | non- | At5g39760 | tyrosine | ARA_LEAF | p-PcUBI | LC | 53 | 87 |
| | targeted | | | | | | | |
| 69574 | non- | At5g39950 | serine | ARA_LEAF | p-PcUBI | GC | 25 | 57 |
| | targeted | | | | | | | |
| 5318 | non- | At5g57050 | alanine | ARA_LEAF | p-PcUBI | GC | 31 | 116 |
| | targeted | | | | | | | |
| 5318 | non- | At5g57050 | citrulline | ARA_LEAF | p-PcUBI | LC | 42 | 54 |
| | targeted | | | | | | | |
| 25498 | non- | At5g59220 | citrulline | ARA_LEAF | p-PcUBI | LC | 41 | 122 |
| | targeted | | | | | | | |
| 70006 | non- | At5g60110 | phenyl- | ARA_LEAF | p-PcUBI | LC | 34 | 70 |
| | targeted | | alanine | | | | | |
| 70006 | non- | At5g60110 | tyrosine | ARA_LEAF | p-PcUBI | LC | 47 | 170 |
| | targeted | | | | | | | |
| 70038 | plastidic | At5g63680 | alanine | ARA_LEAF | p-PcUBI | GC | 42 | 264 |
| 5493 | non- | At5g64920 | alanine | ARA_LEAF | p-PcUBI | GC | 22 | 41 |
| | targeted | | | | | | | |
| 5493 | non- | At5g64920 | Phenyl- | ARA_LEAF | p-PcUBI | GC | 27 | 106 |
| | targeted | | alanine | | | | | |
| 5493 | non- | At5g64920 | serine | ARA_LEAF | p-PcUBI | GC | 29 | 82 |
| | targeted | | | | | | | |
| 5493 | non- | At5g64920 | 5-oxo- | ARA_LEAF | p-PcUBI | GC | 44 | 151 |
| | targeted | | proline | | | | | |
| 70583 | non- | Avin- | aspartate | ARA_LEAF | p-PcUBI | GC | 53 | 163 |
| | targeted | DRAFT_0035 | | | | | | |
| 70583 | non- | Avin- | 5-oxo- | ARA_LEAF | p-PcUBI | GC | 48 | 123 |
| | targeted | DRAFT_0035 | proline | | | | | |
| 70715 | non- | AVIN- | serine | ARA_LEAF | p-PcUBI | GC | 30 | 52 |
| | targeted | DRAFT_0539 | | | | | | |
| 25676 | non- | Avin- | serine | ARA_LEAF | p-PcUBI | GC | 26 | 49 |
| | targeted | DRAFT_1045 | | | | | | |
| 25780 | non- | Avin- | aspartate | ARA_LEAF | p-PcUBI | GC | 49 | 101 |
| | targeted | DRAFT_1398 | | | | | | |
| 5557 | non- | Avin- | alanine | ARA_LEAF | p-PcUBI | GC | 24 | 45 |
| | targeted | DRAFT_1495 | | | | | | |
| 5557 | non- | Avin- | phenyl- | ARA_LEAF | p-PcUBI | GC | 25 | 197 |
| | targeted | DRAFT_1495 | alanine | | | | | |
| 5557 | non- | Avin- | serine | ARA_LEAF | p-PcUBI | GC | 35 | 357 |
| | targeted | DRAFT_1495 | | | | | | |
| 26196 | non- | Avin- | alanine | ARA_LEAF | p-PcUBI | GC | 20 | 30 |
| | targeted | DRAFT_1624 | | | | | | |
| 26434 | non- | Avin- | alanine | ARA_LEAF | p-PcUBI | GC | 50 | 127 |
| | targeted | DRAFT_1806 | | | | | | |
| 6040 | non- | Avin- | alanine | ARA_LEAF | p-PcUBI | GC | 24 | 36 |
| | targeted | DRAFT_2091 | | | | | | |
| 6040 | non- | Avin- | aspartate | ARA_LEAF | p-PcUBI | GC | 89 | 237 |
| | targeted | DRAFT_2091 | | | | | | |
| 6040 | non- | Avin- | serine | ARA_LEAF | p-PcUBI | GC | 47 | 75 |
| | targeted | DRAFT_2091 | | | | | | |
| 27021 | non- | Avin- | aspartate | ARA_LEAF | p-PcUBI | GC | 83 | 94 |
| | targeted | DRAFT_2344 | | | | | | |
| 27021 | non- | Avin- | phenyl- | ARA_LEAF | p-PcUBI | LC | 57 | 254 |
| | targeted | DRAFT_2344 | alanine | | | | | |
| 27021 | non- | Avin- | serine | ARA_LEAF | p-PcUBI | GC | 32 | 198 |
| | targeted | DRAFT_2344 | | | | | | |
| 27021 | non- | Avin- | tyrosine | ARA_LEAF | p-PcUBI | GC | 42 | 298 |
| | targeted | DRAFT_2344 | | | | | | |
| 27021 | non- | Avin- | 5-oxo- | ARA_LEAF | p-PcUBI | GC | 50 | 194 |
| | targeted | DRAFT_2344 | proline | | | | | |
| 71325 | non- | Avin- | tyrosine | ARA_LEAF | p-PcUBI | GC | 36 | 65 |
| | targeted | DRAFT_2365 | | | | | | |
| 71763 | non- | Avin- | asparagine | ARA_LEAF | p-PcUBI | GC | 62 | 91 |
| | targeted | DRAFT_2369 | | | | | | |
| 27882 | non- | Avin- | tyrosine | ARA_LEAF | p-PcUBI | GC | 59 | 193 |
| | targeted | DRAFT_2521 | | | | | | |
| 28040 | non- | Avin- | aspartate | ARA_LEAF | p-PcUBI | GC | 96 | 314 |
| | targeted | DRAFT_2754 | | | | | | |
| 28040 | non- | Avin- | tyrosine | ARA_LEAF | p-PcUBI | GC | 48 | 95 |
| | targeted | DRAFT_2754 | | | | | | |
| 28040 | non- | Avin- | 5-oxo- | ARA_LEAF | p-PcUBI | GC | 34 | 112 |
| | targeted | DRAFT_2754 | proline | | | | | |
| 6075 | non- | Avin- | aspartate | ARA_LEAF | p-PcUBI | GC | 52 | 251 |
| | targeted | DRAFT_3028 | | | | | | |
| 6075 | non- | Avin- | 5-oxo- | ARA_LEAF | p-PcUBI | GC | 41 | 110 |
| | targeted | DRAFT_3028 | proline | | | | | |
| 72346 | non- | Avin- | serine | ARA_LEAF | p-PcUBI | GC | 28 | 60 |
| | targeted | DRAFT_3135 | | | | | | |
| 29246 | non- | Avin- | serine | ARA_LEAF | p-PcUBI | GC | 34 | 44 |
| | targeted | DRAFT_3186 | | | | | | |
| 29286 | non- | Avin- | phenyl- | ARA_LEAF | p-PcUBI | GC | 45 | 115 |
| | targeted | DRAFT_3209 | alanine | | | | | |
| 29500 | non- | Avin- | aspartate | ARA_LEAF | p-PcUBI | GC | 60 | 110 |
| | targeted | DRAFT_3253 | | | | | | |
| 29500 | non- | Avin- | tyrosine | ARA_LEAF | p-PcUBI | LC | 39 | 99 |
| | targeted | DRAFT_3253 | | | | | | |
| 30039 | non- | Avin- | citrulline | ARA_LEAF | p-PcUBI | LC | 30 | 54 |
| | targeted | DRAFT_3556 | | | | | | |
| 72520 | non- | AVIN- | asparagine | ARA_LEAF | p-PcUBI | GC | 49 | 246 |
| | targeted | DRAFT_4128 | | | | | | |
| 31717 | non- | Avin- | aspartate | ARA_LEAF | p-PcUBI | GC | 75 | 152 |
| | targeted | DRAFT_4384 | | | | | | |
| 72935 | non- | Avin- | glycine | ARA_LEAF | p-PcUBI | GC | 68 | 241 |
| | targeted | DRAFT_4385 | | | | | | |
| 73038 | non- | Avin- | phenyl- | ARA_LEAF | p-PcUBI | GC | 27 | 111 |
| | targeted | DRAFT_4420 | alanine | | | | | |
| 73284 | non- | Avin- | alanine | ARA_LEAF | p-PcUBI | GC | 19 | 35 |
| | targeted | DRAFT_4446 | | | | | | |
| 31926 | non- | Avin- | asparagine | ARA_LEAF | p-PcUBI | GC | 41 | 243 |
| | targeted | DRAFT_4562 | | | | | | |
| 31926 | non- | Avin- | phenylalanine | ARA_LEAF | p-PcUBI | GC | 34 | 69 |
| | targeted | DRAFT_4562 | | | | | | |
| 73719 | non- | Avin- | tyrosine | ARA_LEAF | p-PcUBI | GC | 36 | 126 |
| | targeted | DRAFT_4606 | | | | | | |
| 74163 | non- | Avin- | phenyl- | ARA_LEAF | p-PcUBI | LC | 78 | 218 |
| | targeted | DRAFT_4836 | alanine | | | | | |
| 74163 | non- | Avin- | tyrosine | ARA_LEAF | p-PcUBI | LC | 42 | 162 |
| | targeted | DRAFT_4836 | | | | | | |
| 74452 | non- | Avin- | 5-oxo- | ARA_LEAF | p-PcUBI | GC | 32 | 62 |
| | targeted | DRAFT_4989 | proline | | | | | |
| 6510 | non- | Avin- | phenyl- | ARA_LEAF | p-PcUBI | GC | 26 | 3660 |
| | targeted | DRAFT_5103 | alanine | | | | | |
| 6510 | non- | Avin- | tyrosine | ARA_LEAF | p-PcUBI | GC | 51 | 3096 |
| | targeted | DRAFT_5103 | | | | | | |
| 6510 | non- | Avin- | 5-oxo- | ARA_LEAF | p-PcUBI | GC | 34 | 205 |
| | targeted | DRAFT_5103 | proline | | | | | |
| 32037 | non- | Avin- | aspartate | ARA_LEAF | p-PcUBI | GC | 106 | 126 |
| | targeted | DRAFT_5246 | | | | | | |
| 32037 | non- | Avin- | serine | ARA_LEAF | p-PcUBI | GC | 35 | 147 |
| | targeted | DRAFT_5246 | | | | | | |
| 32037 | non- | Avin- | tyrosine | ARA_LEAF | p-PcUBI | GC | 41 | 131 |
| | targeted | DRAFT_5246 | | | | | | |
| 32308 | non- | Avin- | phenyl- | ARA_LEAF | p-PcUBI | GC | 36 | 246 |
| | targeted | DRAFT_5292 | alanine | | | | | |
| 32308 | non- | Avin- | tyrosine | ARA_LEAF | p-PcUBI | GC | 35 | 324 |
| | targeted | DRAFT_5292 | | | | | | |
| 32648 | non- | Avin- | serine | ARA_LEAF | p-PcUBI | GC | 47 | 78 |
| | targeted | DRAFT_5467 | | | | | | |
| 74662 | non- | Avin- | aspartate | ARA_LEAF | p-PcUBI | GC | 56 | 110 |
| | targeted | DRAFT_5579 | | | | | | |
| 33085 | non- | Avin- | aspartate | ARA_LEAF | p-PcUBI | GC | 52 | 122 |
| | targeted | DRAFT_5644 | | | | | | |
| 33596 | non- | Avin- | phenyl- | ARA_LEAF | p-PcUBI | GC | 39 | 90 |
| | targeted | DRAFT_6093 | alanine | | | | | |
| 74729 | non- | Avin- | phenyl- | ARA_LEAF | p-PcUBI | LC | 46 | 143 |
| | targeted | DRAFT_6679 | alanine | | | | | |
| 74729 | non- | Avin- | tyrosine | ARA_LEAF | p-PcUBI | LC | 50 | 143 |
| | targeted | DRAFT_6679 | | | | | | |
| 34301 | non- | AX653549 | citrulline | ARA_LEAF | p-PcUBI | LC | 39 | 48 |
| | targeted | | | | | | | |
| 34301 | non- | AX653549 | 5-oxo- | ARA_LEAF | p-PcUBI | GC | 43 | 93 |
| | targeted | | proline | | | | | |
| 34602 | non- | AY087308 | aspartate | ARA_LEAF | p-PcUBI | GC | 57 | 119 |
| | targeted | | | | | | | |
| 34602 | non- | AY087308 | 5-oxo- | ARA_LEAF | p-PcUBI | GC | 36 | 71 |
| | targeted | | proline | | | | | |
| 34889 | plastidic | B0004 | alanine | ARA_LEAF | p-Super | GC | 29 | 147 |
| 34889 | plastidic | B0004 | phenyl- | ARA_LEAF | p-Super | LC | 30 | 200 |
| | | | alanine | | | | | |
| 34889 | plastidic | B0004 | serine | ARA_LEAF | p-Super | GC | 23 | 195 |
| 34889 | plastidic | B0004 | tyrosine | ARA_LEAF | p-Super | GC | 37 | 458 |
| 34889 | plastidic | B0004 | 5-oxo- | ARA_LEAF | p-Super | GC | 44 | 1268 |
| | | | proline | | | | | |
| 6818 | non- | B0078 | phenyl- | ARA_SEED_2 | p-USP | GC | 32 | 59 |
| | targeted | | alanine | | | | | |
| 35482 | non- | B0124 | glycine | ARA_SEED_2 | p-USP | GC | 31 | 179 |
| | targeted | | | | | | | |
| 35482 | non- | B0124 | phenyl- | ARA_LEAF | p-Super | GC | 36 | 557 |
| | targeted | | alanine | | | | | |
| 35482 | non- | B0124 | serine | ARA_SEED_2 | p-USP | GC | 55 | 171 |
| | targeted | | | | | | | |
| 35482 | non- | B0124 | tyrosine | ARA_LEAF | p-Super | GC | 36 | 265 |
| | targeted | | | | | | | |
| 74986 | non- | B0131 | asparagine | ARA_LEAF | p-Super | GC | 49 | 85 |
| | targeted | | | | | | | |
| 74986 | non- | B0131 | serine | ARA_LEAF | p-Super | GC | 23 | 55 |
| | targeted | | | | | | | |
| 7081 | non- | B0161 | alanine | ARA_LEAF | p-Super | GC | 19 | 58 |
| | targeted | | | | | | | |
| 7081 | non- | B0161 | serine | ARA_LEAF | p-Super | GC | 42 | 103 |
| | targeted | | | | | | | |
| 7081 | non- | B0161 | tyrosine | ARA_LEAF | p-Super | GC | 277 | 1628 |
| | targeted | | | | | | | |
| 35590 | non- | B0221 | serine | ARA_LEAF | p-Super | GC | 25 | 63 |
| | targeted | | | | | | | |
| 35590 | non- | B0221 | tyrosine | ARA_LEAF | p-Super | GC | 39 | 107 |
| | targeted | | | | | | | |
| 35733 | plastidic | B0344 | tyrosine | ARA_SEED_2 | p-USP | GC | 34 | 103 |
| 35733 | plastidic | B0344 | 5-oxo- | ARA_SEED_2 | p-USP | GC | 31 | 40 |
| | | | proline | | | | | |
| 7269 | plastidic | B0348 | glycine | ARA_SEED_2 | p-USP | GC | 96 | 301 |
| 7269 | plastidic | B0348 | phenyl- | ARA_SEED_2 | p-USP | GC | 72 | 197 |
| | | | alanine | | | | | |
| 7269 | plastidic | B0348 | serine | ARA_SEED_2 | p-USP | GC | 38 | 320 |
| 7269 | plastidic | B0348 | tyrosine | ARA_SEED_2 | p-USP | GC | 67 | 83 |
| 75195 | plastidic | B0391 | aspartate | ARA_LEAF | p-Super | GC | 56 | 93 |
| 7333 | non- | B0449 | asparagine | ARA_LEAF | p-Super | GC | 51 | 684 |
| | targeted | | | | | | | |
| 7333 | non- | B0449 | aspartate | ARA_LEAF | p-Super | GC | 88 | 201 |
| | targeted | | | | | | | |
| 7333 | non- | B0449 | phenyl- | ARA_LEAF | p-Super | GC | 40 | 1462 |
| | targeted | | alanine | | | | | |
| 7333 | non- | B0449 | serine | ARA_LEAF | p-Super | GC | 29 | 113 |
| | targeted | | | | | | | |
| 7333 | non- | B0449 | tyrosine | ARA_LEAF | p-Super | GC | 46 | 1202 |
| | targeted | | | | | | | |
| 35875 | non- | B0456 | alanine | ARA_LEAF | p-Super | GC | 20 | 53 |
| | targeted | | | | | | | |
| 7686 | non- | B0486 | aspartate | ARA_LEAF | p-Super | GC | 75 | 129 |
| | targeted | | | | | | | |
| 7686 | non- | B0486 | phenyl- | ARA_LEAF | p-Super | LC | 35 | 156 |
| | targeted | | alanine | | | | | |
| 7686 | non- | B0486 | tyrosine | ARA_LEAF | p-Super | LC | 57 | 227 |
| | targeted | | | | | | | |
| 35936 | non- | B0518 | homo- | ARA_LEAF | p-Super | GC | 24 | 72 |
| | targeted | | serine | | | | | |
| 35967 | plastidic | B0593 | aspartate | ARA_LEAF | p-Super | GC | 84 | 319 |
| 35967 | plastidic | B0593 | phenyl- | ARA_LEAF | p-Super | LC | 37 | 256 |
| | | | alanine | | | | | |
| 35967 | plastidic | B0593 | serine | ARA_LEAF | p-Super | GC | 153 | 359 |
| 35967 | plastidic | B0593 | tyrosine | ARA_LEAF | p-Super | LC | 49 | 202 |
| 35967 | plastidic | B0593 | 3,4-di- | ARA_LEAF | p-Super | GC | 625 | 4899 |
| | | | hydroxy | | | | | |
| | | | phenyl- | | | | | |
| | | | alanine | | | | | |
| | | | (dopa) | | | | | |
| 35967 | plastidic | B0593 | 5-oxo- | ARA_LEAF | p-Super | GC | 100 | 636 |
| | | | proline | | | | | |
| 36114 | non- | B0752 | tyrosine | ARA_LEAF | p-Super | GC | 38 | 112 |
| | targeted | | | | | | | |
| 75286 | plastidic | B0754 | tyrosine | ARA_LEAF | p-USP | LC | 55 | 197 |
| 75807 | non- | B0801 | tyrosine | ARA_LEAF | p-Super | GC | 38 | 67 |
| | targeted | | | | | | | |
| 36299 | non- | B0828 | alanine | ARA_LEAF | p-Super | GC | 24 | 37 |
| | targeted | | | | | | | |
| 75873 | non- | B0841 | glycine | ARA_LEAF | p-Super | GC | 40 | 72 |
| | targeted | | | | | | | |
| 75873 | non- | B0841 | homo- | ARA_LEAF | p-Super | GC | 25 | 59 |
| | targeted | | serine | | | | | |
| 7917 | non- | B0898 | phenyl- | ARA_LEAF | p-Super | GC | 29 | 2908 |
| | targeted | | alanine | | | | | |
| 7917 | non- | B0898 | serine | ARA_LEAF | p-Super | GC | 34 | 290 |
| | targeted | | | | | | | |
| 7917 | non- | B0898 | tyrosine | ARA_LEAF | p-Super | GC | 41 | 2243 |
| | targeted | | | | | | | |
| 7917 | non- | B0898 | 5-oxo- | ARA_LEAF | p-Super | GC | 99 | 411 |
| | targeted | | proline | | | | | |
| 75915 | non- | B0917 | citrulline | ARA_LEAF | p-Super | LC | 32 | 58 |
| | targeted | | | | | | | |
| 75915 | non- | B0917 | glycine | ARA_LEAF | p-Super | GC | 60 | 65 |
| | targeted | | | | | | | |
| 75915 | non- | B0917 | phenyl- | ARA_LEAF | p-Super | GC | 32 | 68 |
| | targeted | | alanine | | | | | |
| 75915 | non- | B0917 | 5-oxo- | ARA_LEAF | p-Super | GC | 41 | 96 |
| | targeted | | proline | | | | | |
| 36670 | non- | B0963 | alanine | ARA_LEAF | p-Super | GC | 20 | 51 |
| | targeted | | | | | | | |
| 36670 | non- | B0963 | phenyl- | ARA_LEAF | p-Super | GC | 58 | 241 |
| | targeted | | alanine | | | | | |
| 36670 | non- | B0963 | tyrosine | ARA_LEAF | p-Super | GC | 46 | 453 |
| | targeted | | | | | | | |
| 36809 | non- | B0980 | phenyl- | ARA_SEED_2 | p-USP | GC | 32 | 89 |
| | targeted | | alanine | | | | | |
| 7941 | non- | B1003 | alanine | ARA_LEAF | p-Super | GC | 51 | 123 |
| | targeted | | | | | | | |
| 7941 | non- | B1003 | aspartate | ARA_LEAF | p-Super | GC | 60 | 317 |
| | targeted | | | | | | | |
| 7941 | non- | B1003 | phenyl- | ARA_LEAF | p-Super | GC | 72 | 728 |
| | targeted | | alanine | | | | | |
| 7941 | non- | B1003 | serine | ARA_LEAF | p-Super | GC | 33 | 327 |
| | targeted | | | | | | | |
| 7941 | non- | B1003 | tyrosine | ARA_LEAF | p-Super | GC | 119 | 664 |
| | targeted | | | | | | | |
| 7941 | non- | B1003 | 3,4-di- | ARA_LEAF | p-Super | GC | 36 | 327 |
| | targeted | | hydroxy | | | | | |
| | | | phenyl- | | | | | |
| | | | alanine | | | | | |
| | | | (dopa) | | | | | |
| 7941 | non- | B1003 | 5-oxo- | ARA_LEAF | p-Super | GC | 34 | 935 |
| | targeted | | proline | | | | | |
| 36880 | non- | B1023 | citrulline | ARA_LEAF | p-Super | LC | 44 | 123 |
| | targeted | | | | | | | |
| 36880 | non- | B1023 | 5-oxo- | ARA_LEAF | p-Super | GC | 38 | 138 |
| | targeted | | proline | | | | | |
| 36907 | non- | B1024 | aspartate | ARA_LEAF | p-Super | GC | 63 | 105 |
| | targeted | | | | | | | |
| 76032 | plastidic | B1065 | aspartate | ARA_LEAF | p-Super | GC | 53 | 132 |
| 76032 | plastidic | B1065 | tyrosine | ARA_LEAF | p-Super | GC | 34 | 45 |
| 37390 | non- | B1137 | serine | ARA_LEAF | p-Super | GC | 27 | 91 |
| | targeted | | | | | | | |
| 76068 | non- | B1155 | phenyl- | ARA_LEAF | p-Super | GC | 34 | 71 |
| | targeted | | alanine | | | | | |
| 37394 | non- | B1163 | phenyl- | ARA_LEAF | p-Super | GC | 30 | 38 |
| | targeted | | alanine | | | | | |
| 37394 | non- | B1163 | tyrosine | ARA_LEAF | p-Super | LC | 39 | 45 |
| | targeted | | | | | | | |
| 76087 | plastidic | B1179 | citrulline | ARA_LEAF | p-Super | LC | 35 | 47 |
| 37400 | non- | B1186 | alanine | ARA_LEAF | p-Super | GC | 28 | 76 |
| | targeted | | | | | | | |
| 37400 | non- | B1186 | serine | ARA_LEAF | p-Super | GC | 46 | 113 |
| | targeted | | | | | | | |
| 76138 | non- | B1205 | citrulline | ARA_LEAF | p-Super | LC | 35 | 77 |
| | targeted | | | | | | | |
| 76151 | non- | B1229 | phenyl- | ARA_LEAF | p-Super | GC | 35 | 44 |
| | targeted | | alanine | | | | | |
| 37483 | non- | B1255 | tyrosine | ARA_LEAF | p-Super | GC | 52 | 124 |
| | targeted | | | | | | | |
| 37503 | non- | B1259 | citrulline | ARA_LEAF | p-Super | LC | 32 | 142 |
| | targeted | | | | | | | |
| 37503 | non- | B1259 | 5-oxo- | ARA_LEAF | p-Super | GC | 38 | 91 |
| | targeted | | proline | | | | | |
| 76157 | plastidic | B1262 | tyrosine | ARA_SEED_2 | p-USP | GC | 27 | 79 |
| 76240 | plastidic | B1264 | phenyl- | ARA_LEAF | p-Super | GC | 33 | 47 |
| | | | alanine | | | | | |
| 37573 | non- | B1280 | glycine | ARA_LEAF | p-Super | GC | 38 | 169 |
| | targeted | | | | | | | |
| 37573 | non- | B1280 | serine | ARA_LEAF | p-Super | GC | 37 | 48 |
| | targeted | | | | | | | |
| 37573 | non- | B1280 | 5-oxo- | ARA_LEAF | p-Super | GC | 38 | 86 |
| | targeted | | proline | | | | | |
| 37658 | plastidic | B1297 | alanine | ARA_SEED_2 | p-USP | GC | 62 | 345 |
| 37658 | plastidic | B1297 | glycine | ARA_SEED_2 | p-USP | GC | 26 | 507 |
| 37807 | non- | B1300 | phenyl- | ARA_LEAF | p-Super | LC | 52 | 109 |
| | targeted | | alanine | | | | | |
| 37807 | non- | B1300 | tyrosine | ARA_LEAF | p-Super | LC | 59 | 115 |
| | targeted | | | | | | | |
| 76867 | non- | B1310 | citrulline | ARA_LEAF | p-Super | LC | 33 | 51 |
| | targeted | | | | | | | |
| 38226 | non- | B1330 | citrulline | ARA_LEAF | p-Super | LC | 33 | 162 |
| | targeted | | | | | | | |
| 76890 | non- | B1343 | phenyl- | ARA_LEAF | p-Super | GC | 27 | 100 |
| | targeted | | alanine | | | | | |
| 77768 | non- | B1348 | alanine | ARA_LEAF | p-Super | GC | 23 | 36 |
| | targeted | | | | | | | |
| 77768 | non- | B1348 | glycine | ARA_LEAF | p-Super | GC | 47 | 134 |
| | targeted | | | | | | | |
| 38266 | plastidic | B1431 | 5-oxo- | ARA_LEAF | p-Super | GC | 37 | 46 |
| | | | proline | | | | | |
| 38289 | non- | B1445 | alanine | ARA_LEAF | p-Super | GC | 19 | 46 |
| | targeted | | | | | | | |
| 38289 | non- | B1445 | citrulline | ARA_LEAF | p-Super | LC | 32 | 96 |
| | targeted | | | | | | | |
| 38289 | non- | B1445 | glycine | ARA_LEAF | p-Super | GC | 37 | 82 |
| | targeted | | | | | | | |
| 38289 | non- | B1445 | phenyl- | ARA_LEAF | p-Super | LC | 34 | 58 |
| | targeted | | alanine | | | | | |
| 38289 | non- | B1445 | serine | ARA_LEAF | p-Super | GC | 37 | 78 |
| | targeted | | | | | | | |
| 77774 | non- | B1479 | alanine | ARA_LEAF | p-Super | GC | 27 | 184 |
| | targeted | | | | | | | |
| 77774 | non- | B1479 | glycine | ARA_SEED_2 | p-USP | GC | 45 | 211 |
| | targeted | | | | | | | |
| 77774 | non- | B1479 | phenyl- | ARA_SEED_2 | p-USP | GC | 35 | 155 |
| | targeted | | alanine | | | | | |
| 77774 | non- | B1479 | serine | ARA_SEED_2 | p-USP | GC | 29 | 287 |
| | targeted | | | | | | | |
| 77774 | non- | B1479 | tyrosine | ARA_SEED_2 | p-USP | GC | 32 | 107 |
| | targeted | | | | | | | |
| 7947 | non- | B1522 | asparagine | ARA_LEAF | p-Super | GC | 42 | 1317 |
| | targeted | | | | | | | |
| 7947 | non- | B1522 | aspartate | ARA_LEAF | p-Super | GC | 66 | 168 |
| | targeted | | | | | | | |
| 7947 | non- | B1522 | phenyl- | ARA_LEAF | p-Super | GC | 34 | 1168 |
| | targeted | | alanine | | | | | |
| 7947 | non- | B1522 | serine | ARA_LEAF | p-Super | GC | 23 | 109 |
| | targeted | | | | | | | |
| 7947 | non- | B1522 | tyrosine | ARA_LEAF | p-Super | GC | 38 | 627 |
| | targeted | | | | | | | |
| 7947 | non- | B1522 | 5-oxo- | ARA_LEAF | p-Super | GC | 47 | 133 |
| | targeted | | proline | | | | | |
| 78138 | non- | B1583 | tyrosine | ARA_LEAF | p-Super | GC | 51 | 184 |
| | targeted | | | | | | | |
| 78154 | non- | B1589 | aspartate | ARA_LEAF | p-Super | GC | 52 | 74 |
| | targeted | | | | | | | |
| 38300 | non- | B1597 | phenyl- | ARA_LEAF | p-Super | GC | 65 | 190 |
| | targeted | | alanine | | | | | |
| 38300 | non- | B1597 | tyrosine | ARA_LEAF | p-Super | GC | 64 | 143 |
| | targeted | | | | | | | |
| 78265 | non- | B1651 | alanine | ARA_LEAF | p-Super | GC | 19 | 39 |
| | targeted | | | | | | | |
| 78753 | non- | B1670 | aspartate | ARA_LEAF | p-Super | GC | 58 | 193 |
| | targeted | | | | | | | |
| 78771 | non- | B1672 | aspartate | ARA_LEAF | p-Super | GC | 59 | 111 |
| | targeted | | | | | | | |
| 78779 | plastidic | B1747 | alanine | ARA_SEED_2 | p-USP | GC | 70 | 193 |
| 78852 | non- | B1755 | aspartate | ARA_LEAF | p-Super | GC | 52 | 103 |
| | targeted | | | | | | | |
| 78852 | non- | B1755 | tyrosine | ARA_LEAF | p-Super | GC | 51 | 120 |
| | targeted | | | | | | | |
| 78883 | non- | B1791 | aspartate | ARA_LEAF | p-Super | GC | 54 | 114 |
| | targeted | | | | | | | |
| 78953 | non- | B1837 | aspartate | ARA_LEAF | p-Super | GC | 51 | 69 |
| | targeted | | | | | | | |
| 8316 | non- | B1838 | aspartate | ARA_LEAF | p-Super | GC | 70 | 104 |
| | targeted | | | | | | | |
| 38573 | non- | B1845 | aspartate | ARA_LEAF | p-Super | GC | 57 | 199 |
| | targeted | | | | | | | |
| 38573 | non- | B1845 | serine | ARA_LEAF | p-Super | GC | 49 | 125 |
| | targeted | | | | | | | |
| 8363 | plastidic | B1854 | asparagine | ARA_SEED_2 | p-USP | GC | 32 | 157 |
| 78970 | non- | B1873 | tyrosine | ARA_LEAF | p-Super | GC | 58 | 217 |
| | targeted | | | | | | | |
| 79056 | non- | B1897 | phenyl- | ARA_LEAF | p-Super | GC | 28 | 196 |
| | targeted | | alanine | | | | | |
| 79056 | non- | B1897 | tyrosine | ARA_LEAF | p-Super | GC | 36 | 211 |
| | targeted | | | | | | | |
| 38767 | non- | B1898 | serine | ARA_LEAF | p-Super | GC | 28 | 106 |
| | targeted | | | | | | | |
| 79181 | non- | B1930 | serine | ARA_LEAF | p-Super | GC | 42 | 113 |
| | targeted | | | | | | | |
| 38899 | non- | B1981 | aspartate | ARA_LEAF | p-Super | GC | 66 | 90 |
| | targeted | | | | | | | |
| 79217 | non- | B2027 | tyrosine | ARA_LEAF | p-Super | GC | 56 | 437 |
| | targeted | | | | | | | |
| 8920 | non- | B2032 | aspartate | ARA_LEAF | p-Super | GC | 64 | 210 |
| | targeted | | | | | | | |
| 38947 | non- | B2063 | aspartate | ARA_LEAF | p-Super | GC | 56 | 184 |
| | targeted | | | | | | | |
| 38947 | non- | B2063 | phenyl- | ARA_LEAF | p-Super | GC | 32 | 1359 |
| | targeted | | alanine | | | | | |
| 38947 | non- | B2063 | serine | ARA_LEAF | p-Super | GC | 27 | 129 |
| | targeted | | | | | | | |
| 38947 | non- | B2063 | tyrosine | ARA_LEAF | p-Super | GC | 36 | 750 |
| | targeted | | | | | | | |
| 8937 | mitochondrial | B2066 | 5-oxo- | ARA_LEAF | p-Super | GC | 34 | 113 |
| | | | proline | | | | | |
| 39002 | non- | B2107 | aspartate | ARA_LEAF | p-Super | GC | 64 | 278 |
| | targeted | | | | | | | |
| 39040 | non- | B2178 | aspartate | ARA_LEAF | p-Super | GC | 58 | 132 |
| | targeted | | | | | | | |
| 39040 | non- | B2178 | citrulline | ARA_LEAF | p-Super | LC | 43 | 94 |
| | targeted | | | | | | | |
| 39040 | non- | B2178 | phenyl- | ARA_LEAF | p-Super | GC | 25 | 44 |
| | targeted | | alanine | | | | | |
| 39040 | non- | B2178 | serine | ARA_LEAF | p-Super | GC | 26 | 64 |
| | targeted | | | | | | | |
| 39040 | non- | B2178 | tyrosine | ARA_LEAF | p-Super | GC | 38 | 39 |
| | targeted | | | | | | | |
| 79279 | plastidic | B2242 | glycine | ARA_SEED_2 | p-USP | GC | 30 | 52 |
| 39120 | non- | B2281 | asparagine | ARA_SEED_2 | p-USP | GC | 34 | 63 |
| | targeted | | | | | | | |
| 39219 | non- | B2360 | aspartate | ARA_LEAF | p-Super | GC | 68 | 124 |
| | targeted | | | | | | | |
| 79342 | plastidic | B2366 | serine | ARA_LEAF | p-Super | GC | 24 | 67 |
| 39237 | non- | B2399 | tyrosine | ARA_LEAF | p-Super | GC | 37 | 156 |
| | targeted | | | | | | | |
| 39237 | non- | B2399 | 3,4-di- | ARA_LEAF | p-Super | GC | 44 | 168 |
| | targeted | | hydroxy | | | | | |
| | | | phenyl- | | | | | |
| | | | alanine | | | | | |
| | | | (dopa) | | | | | |
| 39237 | non- | B2399 | 5-oxo- | ARA_LEAF | p-Super | GC | 48 | 144 |
| | targeted | | proline | | | | | |
| 39255 | non- | B2405 | alanine | ARA_LEAF | p-Super | GC | 28 | 51 |
| | targeted | | | | | | | |
| 39255 | non- | B2405 | aspartate | ARA_LEAF | p-Super | GC | 52 | 87 |
| | targeted | | | | | | | |
| 79398 | non- | B2429 | phenyl- | ARA_LEAF | p-Super | GC | 37 | 52 |
| | targeted | | alanine | | | | | |
| 40329 | non- | B2474 | citrulline | ARA_LEAF | p-Super | LC | 31 | 119 |
| | targeted | | | | | | | |
| 40329 | non- | B2474 | glycine | ARA_LEAF | p-Super | GC | 41 | 71 |
| | targeted | | | | | | | |
| 79509 | non- | B2482 | serine | ARA_LEAF | p-Super | GC | 24 | 62 |
| | targeted | | | | | | | |
| 79509 | non- | B2482 | 5-oxo- | ARA_LEAF | p-Super | GC | 40 | 97 |
| | targeted | | proline | | | | | |
| 9167 | non- | B2513 | phenyl- | ARA_LEAF | p-Super | GC | 65 | 206 |
| | targeted | | alanine | | | | | |
| 9167 | non- | B2513 | tyrosine | ARA_LEAF | p-Super | GC | 38 | 456 |
| | targeted | | | | | | | |
| 79627 | non- | B2551 | serine | ARA_SEED_2 | p-USP | GC | 28 | 120 |
| | targeted | | | | | | | |
| 80521 | plastidic | B2600 | phenyl- | ARA_SEED_2 | p-USP | GC | 40 | 334 |
| | | | alanine | | | | | |
| 40665 | non- | B2613 | alanine | ARA_LEAF | p-Super | GC | 27 | 27 |
| | targeted | | | | | | | |
| 40726 | non- | B2634 | phenyl- | ARA_LEAF | p-Super | GC | 28 | 421 |
| | targeted | | alanine | | | | | |
| 40726 | non- | B2634 | tyrosine | ARA_LEAF | p-Super | GC | 35 | 1033 |
| | targeted | | | | | | | |
| 9244 | non- | B2673 | phenyl- | ARA_LEAF | p-Super | GC | 25 | 121 |
| | targeted | | alanine | | | | | |
| 9244 | non- | B2673 | tyrosine | ARA_LEAF | p-Super | GC | 46 | 58 |
| | targeted | | | | | | | |
| 80589 | non- | B2680 | aspartate | ARA_LEAF | p-Super | GC | 51 | 124 |
| | targeted | | | | | | | |
| 80756 | non- | B2701 | alanine | ARA_LEAF | p-Super | GC | 19 | 36 |
| | targeted | | | | | | | |
| 80756 | non- | B2701 | phenyl- | ARA_LEAF | p-Super | GC | 27 | 317 |
| | targeted | | alanine | | | | | |
| 80756 | non- | B2701 | tyrosine | ARA_LEAF | p-Super | GC | 143 | 237 |
| | targeted | | | | | | | |
| 40741 | non- | B2714 | alanine | ARA_LEAF | p-Super | GC | 26 | 48 |
| | targeted | | | | | | | |
| 80906 | non- | B2739 | alanine | ARA_LEAF | p-Super | GC | 21 | 76 |
| | targeted | | | | | | | |
| 80906 | non- | B2739 | phenyl- | ARA_LEAF | p-Super | GC | 25 | 234 |
| | targeted | | alanine | | | | | |
| 80906 | non- | B2739 | tyrosine | ARA_LEAF | p-Super | GC | 40 | 595 |
| | targeted | | | | | | | |
| 81075 | non- | B2747 | tyrosine | ARA_LEAF | p-Super | GC | 68 | 92 |
| | targeted | | | | | | | |
| 81075 | non- | B2747 | 5-oxo- | ARA_LEAF | p-Super | GC | 43 | 297 |
| | targeted | | proline | | | | | |
| 81451 | non- | B2762 | phenyl- | ARA_LEAF | p-Super | GC | 25 | 78 |
| | targeted | | alanine | | | | | |
| 40795 | non- | B2812 | phenyl- | ARA_LEAF | p-Super | GC | 44 | 99 |
| | targeted | | alanine | | | | | |
| 40795 | non- | B2812 | serine | ARA_LEAF | p-Super | GC | 26 | 123 |
| | targeted | | | | | | | |
| 40795 | non- | B2812 | tyrosine | ARA_LEAF | p-Super | GC | 38 | 122 |
| | targeted | | | | | | | |
| 81576 | non- | B2818 | phenyl- | ARA_LEAF | p-Super | GC | 27 | 826 |
| | targeted | | alanine | | | | | |
| 81576 | non- | B2818 | tyrosine | ARA_LEAF | p-Super | LC | 41 | 427 |
| | targeted | | | | | | | |
| 81576 | non- | B2818 | 5-oxo- | ARA_LEAF | p-Super | GC | 34 | 266 |
| | targeted | | proline | | | | | |
| 81692 | non- | B2828 | aspartate | ARA_SEED_2 | p-USP | GC | 40 | 154 |
| | targeted | | | | | | | |
| 81935 | non- | B2839 | tyrosine | ARA_LEAF | p-Super | LC | 42 | 55 |
| | targeted | | | | | | | |
| 81980 | non- | B2849 | phenyl- | ARA_LEAF | p-Super | GC | 26 | 78 |
| | targeted | | alanine | | | | | |
| 81988 | plastidic | B2913 | glycine | ARA_SEED_2 | p-USP | GC | 31 | 168 |
| 81988 | plastidic | B2913 | serine | ARA_SEED_2 | p-USP | GC | 28 | 215 |
| 9333 | non- | B2923 | aspartate | ARA_LEAF | p-Super | GC | 64 | 126 |
| | targeted | | | | | | | |
| 42046 | non- | B3064 | asparagine | ARA_SEED_2 | p-Super | GC | 30 | 189 |
| | targeted | | | | | | | |
| 82424 | non- | B3083 | phenyl- | ARA_LEAF | p-Super | GC | 28 | 61 |
| | targeted | | alanine | | | | | |
| 82424 | non- | B3083 | serine | ARA_LEAF | p-Super | GC | 23 | 108 |
| | targeted | | | | | | | |
| 82424 | non- | B3083 | tyrosine | ARA_LEAF | p-Super | GC | 35 | 72 |
| | targeted | | | | | | | |
| 82459 | non- | B3098 | alanine | ARA_LEAF | p-Super | GC | 31 | 58 |
| | targeted | | | | | | | |
| 82459 | non- | B3098 | glycine | ARA_LEAF | p-Super | GC | 38 | 76 |
| | targeted | | | | | | | |
| 42471 | non- | B3121 | tyrosine | ARA_LEAF | p-Super | GC | 39 | 73 |
| | targeted | | | | | | | |
| 82510 | plastidic | B3221 | tyrosine | ARA_LEAF | p-Super | LC | 37 | 55 |
| 9470 | non- | B3246 | serine | ARA_LEAF | p-Super | GC | 24 | 101 |
| | targeted | | | | | | | |
| 9470 | non- | B3246 | 5-oxo- | ARA_LEAF | p-Super | GC | 33 | 89 |
| | targeted | | proline | | | | | |
| 9492 | non- | B3256 | asparagine | ARA_SEED_2 | p-USP | GC | 67 | 286 |
| | targeted | | | | | | | |
| 9492 | non- | B3256 | glycine | ARA_SEED_2 | p-USP | GC | 28 | 344 |
| | targeted | | | | | | | |
| 9492 | non- | B3256 | serine | ARA_SEED_2 | p-USP | GC | 31 | 1323 |
| | targeted | | | | | | | |
| 9492 | non- | B3256 | 5-oxo- | ARA_SEED_2 | p-USP | GC | 32 | 175 |
| | targeted | | proline | | | | | |
| 42502 | non- | B3262 | alanine | ARA_LEAF | p-Super | GC | 25 | 58 |
| | targeted | | | | | | | |
| 42502 | non- | B3262 | phenyl- | ARA_LEAF | p-Super | LC | 36 | 199 |
| | targeted | | alanine | | | | | |
| 42502 | non- | B3262 | serine | ARA_LEAF | p-Super | GC | 34 | 141 |
| | targeted | | | | | | | |
| 42502 | non- | B3262 | tyrosine | ARA_LEAF | p-Super | LC | 45 | 322 |
| | targeted | | | | | | | |
| 42502 | non- | B3262 | 5-oxo- | ARA_LEAF | p-Super | GC | 72 | 179 |
| | targeted | | proline | | | | | |
| 10104 | non- | B3346 | alanine | ARA_LEAF | p-Super | GC | 20 | 53 |
| | targeted | | | | | | | |
| 82572 | non- | B3352 | alanine | ARA_LEAF | p-Super | GC | 19 | 45 |
| | targeted | | | | | | | |
| 82572 | non- | B3352 | glycine | ARA_LEAF | p-Super | GC | 40 | 80 |
| | targeted | | | | | | | |
| 82846 | plastidic | B3392 | aspartate | ARA_LEAF | p-Super | GC | 41 | 49 |
| 82866 | non- | B3403 | phenyl- | ARA_LEAF | p-Super | GC | 26 | 63 |
| | targeted | | alanine | | | | | |
| 82866 | non- | B3403 | serine | ARA_LEAF | p-Super | GC | 31 | 45 |
| | targeted | | | | | | | |
| 42559 | non- | B3410 | alanine | ARA_LEAF | p-Super | GC | 23 | 65 |
| | targeted | | | | | | | |
| 42592 | non- | B3509 | serine | ARA_LEAF | p-Super | GC | 26 | 63 |
| | targeted | | | | | | | |
| 83153 | non- | B3533 | aspartate | ARA_SEED_2 | p-USP | GC | 37 | 669 |
| | targeted | | | | | | | |
| 83213 | plastidic | B3607 | phenyl- | ARA_SEED_2 | p-USP | LC | 46 | 60 |
| | | | alanine | | | | | |
| 83213 | plastidic | B3607 | tyrosine | ARA_SEED_2 | p-USP | GC | 29 | 82 |
| 42931 | non- | B3634 | alanine | ARA_LEAF | p-Super | GC | 21 | 39 |
| | targeted | | | | | | | |
| 42931 | non- | B3634 | asparagine | ARA_LEAF | p-Super | GC | 44 | 169 |
| | targeted | | | | | | | |
| 42931 | non- | B3634 | phenyl- | ARA_LEAF | p-Super | GC | 82 | 92 |
| | targeted | | alanine | | | | | |
| 83429 | non- | B3646 | glycine | ARA_LEAF | p-Super | GC | 35 | 1154 |
| | targeted | | | | | | | |
| 83629 | plastidic | B3670 | tyrosine | ARA_SEED_2 | p-USP | GC | 60 | 97 |
| 83657 | plastidic | B3744 | asparagine | ARA_SEED_2 | p-USP | GC | 36 | 2817 |
| 10252 | non- | B3771 | glycine | ARA_SEED_2 | p-USP | GC | 39 | 114 |
| | targeted | | | | | | | |
| 10252 | non- | B3771 | phenyl- | ARA_SEED_2 | p-USP | LC | 69 | 81 |
| | targeted | | alanine | | | | | |
| 83726 | non- | B3791 | aspartate | ARA_LEAF | p-Super | GC | 53 | 158 |
| | targeted | | | | | | | |
| 83726 | non- | B3791 | 5-oxo- | ARA_LEAF | p-Super | GC | 39 | 117 |
| | targeted | | proline | | | | | |
| 43800 | non- | B3793 | phenyl- | ARA_LEAF | p-Super | GC | 26 | 438 |
| | targeted | | alanine | | | | | |
| 43839 | non- | B3813 | phenyl- | ARA_SEED_2 | p-USP | GC | 34 | 85 |
| | targeted | | alanine | | | | | |
| 44196 | non- | B3814 | citrulline | ARA_LEAF | p-Super | LC | 30 | 124 |
| | targeted | | | | | | | |
| 83821 | non- | B3872 | alanine | ARA_LEAF | p-Super | GC | 20 | 44 |
| | targeted | | | | | | | |
| 83821 | plastidic | B3872 | aspartate | ARA_LEAF | p-Super | GC | 55 | 96 |
| 83821 | non- | B3872 | serine | ARA_LEAF | p-Super | GC | 36 | 59 |
| | targeted | | | | | | | |
| 83821 | non- | B3872 | tyrosine | ARA_LEAF | p-Super | GC | 35 | 54 |
| | targeted | | | | | | | |
| 44372 | non- | B3989 | phenyl- | ARA_LEAF | p-Super | GC | 25 | 1035 |
| | targeted | | alanine | | | | | |
| 44372 | non- | B3989 | tyrosine | ARA_LEAF | p-Super | GC | 46 | 762 |
| | targeted | | | | | | | |
| 44378 | non- | B4012 | aspartate | ARA_SEED_2 | p-USP | GC | 51 | 282 |
| | targeted | | | | | | | |
| 44378 | non- | B4012 | 5-oxo- | ARA_SEED_2 | p-USP | GC | 33 | 47 |
| | targeted | | proline | | | | | |
| 83835 | plastidic | B4024 | glycine | ARA_SEED_2 | p-USP | GC | 210 | 262 |
| 83835 | plastidic | B4024 | serine | ARA_SEED_2 | p-USP | GC | 35 | 399 |
| 10740 | non- | B4029 | alanine | ARA_LEAF | p-Super | GC | 22 | 775 |
| | targeted | | | | | | | |
| 10740 | non- | B4029 | aspartate | ARA_LEAF | p-Super | GC | 59 | 231 |
| | targeted | | | | | | | |
| 10740 | non- | B4029 | glycine | ARA_LEAF | p-Super | GC | 39 | 590 |
| | targeted | | | | | | | |
| 10740 | non- | B4029 | phenyl- | ARA_LEAF | p-Super | GC | 28 | 2010 |
| | targeted | | alanine | | | | | |
| 10740 | non- | B4029 | serine | ARA_LEAF | p-Super | GC | 28 | 177 |
| | targeted | | | | | | | |
| 10740 | non- | B4029 | tyrosine | ARA_LEAF | p-Super | GC | 36 | 3270 |
| | targeted | | | | | | | |
| 10740 | non- | B4029 | 5-oxo- | ARA_LEAF | p-Super | GC | 44 | 367 |
| | targeted | | proline | | | | | |
| 44609 | non- | B4121 | serine | ARA_LEAF | p-Super | GC | 23 | 92 |
| | targeted | | | | | | | |
| 45321 | non- | B4256 | aspartate | ARA_LEAF | p-Super | GC | 56 | 155 |
| | targeted | | | | | | | |
| 45321 | non- | B4256 | phenyl- | ARA_LEAF | p-Super | GC | 29 | 242 |
| | targeted | | alanine | | | | | |
| 45321 | non- | B4256 | serine | ARA_LEAF | p-Super | GC | 31 | 227 |
| | targeted | | | | | | | |
| 45321 | non- | B4256 | tyrosine | ARA_LEAF | p-Super | GC | 151 | 280 |
| | targeted | | | | | | | |
| 45757 | non- | C_pp004096192r | serine | ARA_LEAF | p-Super | GC | 32 | 75 |
| | targeted | | | | | | | |
| 84079 | plastidic | D90900 | alanine | ARA_LEAF | p-PcUBI | GC | 20 | 34 |
| 45795 | non- | GM02LC11114 | asparagine | ARA_LEAF | p-PcUBI | GC | 78 | 249 |
| | targeted | | | | | | | |
| 45795 | non- | GM02LC11114 | phenyl- | ARA_LEAF | p-PcUBI | LC | 44 | 76 |
| | targeted | | alanine | | | | | |
| 10811 | non- | GM02LC12622 | alanine | ARA_LEAF | p-PcUBI | GC | 21 | 46 |
| | targeted | | | | | | | |
| 10811 | non- | GM02LC12622 | phenyl- | ARA_LEAF | p-PcUBI | LC | 44 | 218 |
| | targeted | | alanine | | | | | |
| 10811 | non- | GM02LC12622 | tyrosine | ARA_LEAF | p-PcUBI | GC | 45 | 525 |
| | targeted | | | | | | | |
| 45897 | non- | GM02LC15313 | phenyl- | ARA_LEAF | p-PcUBI | LC | 32 | 137 |
| | targeted | | alanine | | | | | |
| 45897 | non- | GM02LC15313 | tyrosine | ARA_LEAF | p-PcUBI | LC | 53 | 84 |
| | targeted | | | | | | | |
| 46405 | non- | GM02LC17485 | aspartate | ARA_LEAF | p-PcUBI | GC | 57 | 118 |
| | targeted | | | | | | | |
| 46515 | non- | GM02LC17556 | phenyl- | ARA_LEAF | p-PcUBI | GC | 26 | 179 |
| | targeted | | alanine | | | | | |
| 46515 | non- | GM02LC17556 | tyrosine | ARA_LEAF | p-PcUBI | LC | 48 | 179 |
| | targeted | | | | | | | |
| 84198 | non- | GM02LC21368 | phenyl- | ARA_LEAF | p-PcUBI | LC | 59 | 383 |
| | targeted | | alanine | | | | | |
| 84198 | non- | GM02LC21368 | serine | ARA_LEAF | p-PcUBI | GC | 26 | 82 |
| | targeted | | | | | | | |
| 84198 | non- | GM02LC21368 | tyrosine | ARA_LEAF | p-PcUBI | GC | 36 | 903 |
| | targeted | | | | | | | |
| 46850 | non- | GM02LC44512 | alanine | ARA_LEAF | p-PcUBI | GC | 29 | 66 |
| | targeted | | | | | | | |
| 46850 | non- | GM02LC44512 | serine | ARA_LEAF | p-PcUBI | GC | 37 | 67 |
| | targeted | | | | | | | |
| 47159 | non- | NZ_AAAU02000016.150 | alanine | ARA_LEAF | p-PcUBI | GC | 21 | 74 |
| | targeted | | | | | | | |
| 47159 | non- | NZ_AAAU02000016.150 | glycine | ARA_LEAF | p-PcUBI | GC | 40 | 103 |
| | targeted | | | | | | | |
| 47159 | non- | NZ_AAAU02000016.150 | serine | ARA_LEAF | p-PcUBI | GC | 24 | 65 |
| | targeted | | | | | | | |
| 59851 | non- | S_pp015018333r | asparagine | ARA_LEAF | p-Super | GC | 43 | 143 |
| | targeted | | | | | | | |
| 59851 | non- | S_pp015018333r | phenyl- | ARA_LEAF | p-Super | GC | 37 | 174 |
| | targeted | | alanine | | | | | |
| 59851 | non- | S_pp015018333r | tyrosine | ARA_LEAF | p-Super | LC | 41 | 102 |
| | targeted | | | | | | | |
| 59851 | non- | S_pp015018333r | 5-oxo- | ARA_LEAF | p-Super | GC | 45 | 114 |
| | targeted | | proline | | | | | |
| 47566 | plastidic | Sll0080 | citrulline | ARA_LEAF | p-PcUBI | LC | 31 | 60 |
| 84265 | plastidic | Sll0228 | asparagine | ARA_LEAF | p-PcUBI | GC | 76 | 319 |
| 84265 | plastidic | Sll0228 | tyrosine | ARA_LEAF | p-PcUBI | GC | 48 | 58 |
| 84441 | plastidic | Sll0240 | alanine | ARA_LEAF | p-PcUBI | GC | 30 | 40 |
| 49143 | plastidic | Sll0248 | phenyl- | ARA_LEAF | p-PcUBI | LC | 33 | 128 |
| | | | alanine | | | | | |
| 49143 | plastidic | Sll0248 | tyrosine | ARA_LEAF | p-PcUBI | LC | 40 | 277 |
| 84865 | plastidic | Sll0281 | alanine | ARA_LEAF | p-PcUBI | GC | 22 | 34 |
| 84869 | plastidic | Sll0378 | serine | ARA_LEAF | p-PcUBI | GC | 24 | 126 |
| 50104 | non- | Sll0420 | phenyl- | ARA_LEAF | p-PcUBI | GC | 31 | 49 |
| | targeted | | alanine | | | | | |
| 50104 | non- | Sll0420 | tyrosine | ARA_LEAF | p-PcUBI | GC | 36 | 94 |
| | targeted | | | | | | | |
| 85510 | plastidic | Sll0593 | citrulline | ARA_LEAF | p-PcUBI | LC | 54 | 140 |
| 50950 | non- | Sll0682 | serine | ARA_LEAF | p-PcUBI | GC | 26 | 90 |
| | targeted | | | | | | | |
| 50950 | non- | Sll0682 | 5-oxo- | ARA_LEAF | p-PcUBI | GC | 34 | 92 |
| | targeted | | proline | | | | | |
| 51198 | non- | Sll0816 | aspartate | ARA_LEAF | p-PcUBI | GC | 52 | 318 |
| | targeted | | | | | | | |
| 51198 | non- | Sll0816 | glycine | ARA_LEAF | p-PcUBI | GC | 37 | 110 |
| | targeted | | | | | | | |
| 51198 | non- | Sll0816 | serine | ARA_LEAF | p-PcUBI | GC | 32 | 198 |
| | targeted | | | | | | | |
| 51198 | non- | Sll0816 | tyrosine | ARA_LEAF | p-PcUBI | GC | 53 | 88 |
| | targeted | | | | | | | |
| 51268 | plastidic | Sll0891 | aspartate | ARA_LEAF | p-PcUBI | GC | 90 | 176 |
| 51268 | plastidic | Sll0891 | phenyl- | ARA_LEAF | p-PcUBI | GC | 37 | 118 |
| | | | alanine | | | | | |
| 51268 | plastidic | Sll0891 | serine | ARA_LEAF | p-PcUBI | GC | 23 | 95 |
| 52246 | mitochondrial | Sll0934 | aspartate | ARA_LEAF | p-PcUBI | GC | 61 | 94 |
| 52364 | non- | Sll0945 | serine | ARA_LEAF | p-PcUBI | GC | 28 | 86 |
| | targeted | | | | | | | |
| 52634 | mitochondrial | Sll1031 | aspartate | ARA_LEAF | p-PcUBI | GC | 61 | 317 |
| 52634 | mitochondrial | Sll1031 | phenyl- | ARA_LEAF | p-PcUBI | LC | 33 | 128 |
| | | | alanine | | | | | |
| 52634 | mitochondrial | Sll1031 | serine | ARA_LEAF | p-PcUBI | GC | 24 | 67 |
| 85723 | mitochondrial | Sll1032 | aspartate | ARA_LEAF | p-PcUBI | GC | 45 | 106 |
| 52660 | plastidic | Sll1056 | serine | ARA_LEAF | p-PcUBI | GC | 24 | 75 |
| 11211 | mitochondrial | Sll1108 | aspartate | ARA_LEAF | p-PcUBI | GC | 55 | 137 |
| 11211 | mitochondrial | Sll1108 | 5-oxo- | ARA_LEAF | p-PcUBI | GC | 39 | 103 |
| | | | proline | | | | | |
| 53456 | non- | Sll1393 | asparagine | ARA_LEAF | p-PcUBI | GC | 55 | 264 |
| | targeted | | | | | | | |
| 53456 | non- | Sll1393 | tyrosine | ARA_LEAF | p-PcUBI | GC | 82 | 129 |
| | targeted | | | | | | | |
| 53878 | mitochondrial | Sll1443 | aspartate | ARA_LEAF | p-PcUBI | GC | 58 | 119 |
| 54337 | mitochondrial | Sll1450 | glycine | ARA_LEAF | p-PcUBI | GC | 36 | 102 |
| 54337 | mitochondrial | Sll1450 | phenyl- | ARA_LEAF | p-PcUBI | LC | 32 | 42 |
| | | | alanine | | | | | |
| 54337 | mitochondrial | Sll1450 | serine | ARA_LEAF | p-PcUBI | GC | 24 | 83 |
| 54337 | mitochondrial | Sll1450 | tyrosine | ARA_LEAF | p-PcUBI | GC | 37 | 92 |
| 54452 | plastidic | Sll1522 | aspartate | ARA_LEAF | p-PcUBI | GC | 50 | 230 |
| 54452 | plastidic | Sll1522 | 5-oxo- | ARA_LEAF | p-PcUBI | GC | 33 | 103 |
| | | | proline | | | | | |
| 11423 | mitochondrial | Sll1545 | serine | ARA_LEAF | p-PcUBI | GC | 26 | 50 |
| 54804 | non- | Sll1546 | aspartate | ARA_LEAF | p-PcUBI | GC | 51 | 102 |
| | targeted | | | | | | | |
| 54804 | non- | Sll1546 | phenylalanine | ARA_LEAF | p-PcUBI | GC | 27 | 53 |
| | targeted | | | | | | | |
| 54804 | non- | Sll1546 | serine | ARA_LEAF | p-PcUBI | GC | 25 | 45 |
| | targeted | | | | | | | |
| 85743 | mitochondrial | Sll1598 | aspartate | ARA_LEAF | p-PcUBI | GC | 55 | 138 |
| 85743 | mitochondrial | Sll1598 | phenyl- | ARA_LEAF | p-PcUBI | GC | 26 | 113 |
| | | | alanine | | | | | |
| 55379 | non- | Sll1761 | serine | ARA_LEAF | p-PcUBI | GC | 23 | 63 |
| | targeted | | | | | | | |
| 55379 | non- | Sll1761 | tyrosine | ARA_LEAF | p-PcUBI | GC | 39 | 141 |
| | targeted | | | | | | | |
| 85808 | mitochondrial | Sll1815 | alanine | ARA_LEAF | p-PcUBI | GC | 31 | 483 |
| 85808 | mitochondrial | Sll1815 | citrulline | ARA_LEAF | p-PcUBI | LC | 39 | 122 |
| 85808 | mitochondrial | Sll1815 | glycine | ARA_LEAF | p-PcUBI | GC | 118 | 238 |
| 85808 | mitochondrial | Sll1815 | serine | ARA_LEAF | p-PcUBI | GC | 55 | 83 |
| 85808 | mitochondrial | Sll1815 | tyrosine | ARA_LEAF | p-PcUBI | GC | 39 | 88 |
| 86447 | plastidic | Sll1848 | glycine | ARA_LEAF | p-PcUBI | GC | 48 | 205 |
| 86447 | plastidic | Sll1848 | phenyl- | ARA_LEAF | p-PcUBI | GC | 26 | 318 |
| | | | alanine | | | | | |
| 86447 | plastidic | Sll1848 | serine | ARA_LEAF | p-PcUBI | GC | 28 | 319 |
| 11471 | mitochondrial | Sll1917 | aspartate | ARA_LEAF | p-PcUBI | GC | 64 | 265 |
| 11471 | mitochondrial | Sll1917 | citrulline | ARA_LEAF | p-PcUBI | LC | 41 | 71 |
| 11471 | mitochondrial | Sll1917 | phenyl- | ARA_LEAF | p-PcUBI | GC | 32 | 56 |
| | | | alanine | | | | | |
| 11471 | mitochondrial | Sll1917 | serine | ARA_LEAF | p-PcUBI | GC | 28 | 69 |
| 11471 | mitochondrial | Sll1917 | tyrosine | ARA_LEAF | p-PcUBI | GC | 35 | 60 |
| 55385 | non- | Sll1920 | aspartate | ARA_LEAF | p-PcUBI | GC | 52 | 204 |
| | targeted | | | | | | | |
| 55385 | mitochondrial | Sll1920 | aspartate | ARA_LEAF | p-PcUBI | GC | 65 | 142 |
| 86508 | plastidic | Sll2003 | glycine | ARA_LEAF | p-PcUBI | GC | 47 | 67 |
| 86540 | mitochondrial | Slr0239 | phenyl- | ARA_LEAF | p-PcUBI | LC | 41 | 279 |
| | | | alanine | | | | | |
| 86540 | mitochondrial | Slr0239 | tyrosine | ARA_LEAF | p-PcUBI | LC | 54 | 214 |
| 86820 | plastidic | Slr0370 | citrulline | ARA_LEAF | p-PcUBI | LC | 38 | 90 |
| 56153 | plastidic | Slr0597 | alanine | ARA_LEAF | p-PcUBI | GC | 28 | 38 |
| 56153 | plastidic | Slr0597 | serine | ARA_LEAF | p-PcUBI | GC | 27 | 104 |
| 56153 | plastidic | Slr0597 | 5-oxo- | ARA_LEAF | p-PcUBI | GC | 40 | 96 |
| | | | proline | | | | | |
| 56894 | mitochondrial | Slr0710 | asparagine | ARA_LEAF | p-PcUBI | GC | 49 | 215 |
| 56894 | mitochondrial | Slr0710 | phenyl- | ARA_LEAF | p-PcUBI | GC | 26 | 99 |
| | | | alanine | | | | | |
| 56894 | mitochondrial | Slr0710 | tyrosine | ARA_LEAF | p-PcUBI | GC | 37 | 80 |
| 87397 | plastidic | Slr0721 | phenyl- | ARA_LEAF | p-PcUBI | GC | 30 | 107 |
| | | | alanine | | | | | |
| 57235 | plastidic | Slr0739 | alanine | ARA_LEAF | p-PcUBI | GC | 19 | 56 |
| 57235 | plastidic | Slr0739 | asparagine | ARA_LEAF | p-PcUBI | GC | 155 | 1305 |
| 57235 | plastidic | Slr0739 | phenyl- | ARA_LEAF | p-PcUBI | GC | 44 | 71 |
| | | | alanine | | | | | |
| 57235 | plastidic | Slr0739 | serine | ARA_LEAF | p-PcUBI | GC | 52 | 166 |
| 57235 | plastidic | Slr0739 | tyrosine | ARA_LEAF | p-PcUBI | GC | 66 | 82 |
| 57235 | plastidic | Slr0739 | 5-oxo- | ARA_LEAF | p-PcUBI | GC | 33 | 130 |
| | | | proline | | | | | |
| 57679 | mitochondrial | Slr0782 | phenyl- | ARA_LEAF | p-PcUBI | LC | 34 | 38 |
| | | | alanine | | | | | |
| 87610 | mitochondrial | Slr0862 | serine | ARA_LEAF | p-PcUBI | GC | 23 | 45 |
| 87655 | plastidic | Slr0966 | tyrosine | ARA_LEAF | p-PcUBI | GC | 43 | 77 |
| 87853 | mitochondrial | Slr1133 | ornithine | ARA_LEAF | p-PcUBI | GC | 69 | 390 |
| 87853 | mitochondrial | Slr1133 | serine | ARA_LEAF | p-PcUBI | GC | 26 | 69 |
| 58058 | non- | Slr1269 | asparagine | ARA_LEAF | p-PcUBI | GC | 45 | 93 |
| | targeted | | | | | | | |
| 58668 | mitochondrial | Slr1492 | aspartate | ARA_LEAF | p-PcUBI | GC | 48 | 139 |
| 58668 | mitochondrial | Slr1492 | phenyl- | ARA_LEAF | p-PcUBI | GC | 31 | 35 |
| | | | alanine | | | | | |
| 88158 | mitochondrial | Slr1509 | phenyl- | ARA_LEAF | p-PcUBI | GC | 43 | 94 |
| | | | alanine | | | | | |
| 88158 | mitochondrial | Slr1509 | tyrosine | ARA_LEAF | p-PcUBI | GC | 67 | 86 |
| 88271 | non- | Slr1511 | phenyl- | ARA_LEAF | p-PcUBI | GC | 30 | 31 |
| | targeted | | alanine | | | | | |
| 88271 | non- | Slr1511 | serine | ARA_LEAF | p-PcUBI | GC | 30 | 71 |
| | targeted | | | | | | | |
| 12070 | mitochondrial | Slr1655 | phenyl- | ARA_LEAF | p-PcUBI | GC | 28 | 60 |
| | | | alanine | | | | | |
| 12070 | mitochondrial | Slr1655 | serine | ARA_LEAF | p-PcUBI | GC | 33 | 83 |
| 12070 | mitochondrial | Slr1655 | tyrosine | ARA_LEAF | p-PcUBI | GC | 35 | 104 |
| 58731 | plastidic | Slr1739 | phenyl- | ARA_LEAF | p-PcUBI | GC | 47 | 192 |
| | | | alanine | | | | | |
| 58731 | plastidic | Slr1739 | serine | ARA_LEAF | p-PcUBI | GC | 59 | 99 |
| 58731 | plastidic | Slr1739 | tyrosine | ARA_LEAF | p-PcUBI | GC | 73 | 163 |
| 58731 | plastidic | Slr1739 | 5-oxo- | ARA_LEAF | p-PcUBI | GC | 46 | 128 |
| | | | proline | | | | | |
| 58751 | mitochondrial | Slr1742 | asparagine | ARA_LEAF | p-PcUBI | GC | 45 | 188 |
| 12140 | mitochondrial | Slr1791 | citrulline | ARA_LEAF | p-PcUBI | LC | 32 | 95 |
| 59041 | non- | Slr1882 | asparagine | ARA_LEAF | p-PcUBI | GC | 65 | 112 |
| | targeted | | | | | | | |
| 12341 | plastidic | Slr2072 | homo- | ARA_LEAF | p-PcUBI | GC | 40 | 86 |
| | | | serine | | | | | |
| 12341 | mitochondrial | Slr2072 | tyrosine | ARA_LEAF | p-PcUBI | LC | 30 | 65 |
| 59370 | plastidic | Slr2124 | alanine | ARA_LEAF | p-PcUBI | GC | 19 | 44 |
| 59370 | plastidic | Slr2124 | phenyl- | ARA_LEAF | p-PcUBI | LC | 36 | 69 |
| | | | alanine | | | | | |
| 59370 | plastidic | Slr2124 | tyrosine | ARA_LEAF | p-PcUBI | GC | 36 | 87 |
| 12698 | non- | TTC0019 | phenyl- | ARA_LEAF | p-PcUBI | GC | 31 | 624 |
| | targeted | | alanine | | | | | |
| 12698 | non- | TTC0019 | tyrosine | ARA_LEAF | p-PcUBI | GC | 157 | 378 |
| | targeted | | | | | | | |
| 12698 | non- | TTC0019 | 5-oxo- | ARA_LEAF | p-PcUBI | GC | 36 | 277 |
| | targeted | | proline | | | | | |
| 88526 | non- | TTC0239 | tyrosine | ARA_LEAF | p-PcUBI | GC | 35 | 136 |
| | targeted | | | | | | | |
| 61070 | non- | TTC0337 | asparagine | ARA_LEAF | p-PcUBI | GC | 44 | 216 |
| | targeted | | | | | | | |
| 61532 | non- | TTC0768 | tyrosine | ARA_LEAF | p-PcUBI | LC | 51 | 97 |
| | targeted | | | | | | | |
| 88875 | non- | TTC0881 | alanine | ARA_LEAF | p-PcUBI | GC | 25 | 40 |
| | targeted | | | | | | | |
| 88875 | non- | TTC0881 | serine | ARA_LEAF | p-PcUBI | GC | 27 | 84 |
| | targeted | | | | | | | |
| 61553 | non- | TTC0917 | alanine | ARA_LEAF | p-PcUBI | GC | 20 | 37 |
| | targeted | | | | | | | |
| 61553 | non- | TTC0917 | serine | ARA_LEAF | p-PcUBI | GC | 23 | 35 |
| | targeted | | | | | | | |
| 61723 | non- | TTC1193 | phenyl- | ARA_LEAF | p-PcUBI | GC | 33 | 133 |
| | targeted | | alanine | | | | | |
| 61723 | non- | TTC1193 | tyrosine | ARA_LEAF | p-PcUBI | LC | 53 | 103 |
| | targeted | | | | | | | |
| 62079 | non- | TTC1386 | alanine | ARA_LEAF | p-PcUBI | GC | 54 | 91 |
| | targeted | | | | | | | |
| 62079 | non- | TTC1386 | glycine | ARA_LEAF | p-PcUBI | GC | 230 | 978 |
| | targeted | | | | | | | |
| 62079 | non- | TTC1386 | phenyl- | ARA_LEAF | p-PcUBI | GC | 45 | 528 |
| | targeted | | alanine | | | | | |
| 62079 | non- | TTC1386 | serine | ARA_LEAF | p-PcUBI | GC | 23 | 117 |
| | targeted | | | | | | | |
| 62079 | non- | TTC1386 | tyrosine | ARA_LEAF | p-PcUBI | GC | 39 | 829 |
| | targeted | | | | | | | |
| 12974 | non- | TTC1550 | alanine | ARA_LEAF | p-PcUBI | GC | 45 | 86 |
| | targeted | | | | | | | |
| 12974 | non- | TTC1550 | phenyl- | ARA_LEAF | p-PcUBI | GC | 54 | 266 |
| | targeted | | alanine | | | | | |
| 12974 | non- | TTC1550 | serine | ARA_LEAF | p-PcUBI | GC | 23 | 104 |
| | targeted | | | | | | | |
| 12974 | non- | TTC1550 | tyrosine | ARA_LEAF | p-PcUBI | GC | 35 | 213 |
| | targeted | | | | | | | |
| 12974 | non- | TTC1550 | 5-oxo- | ARA_LEAF | p-PcUBI | GC | 47 | 171 |
| | targeted | | proline | | | | | |
| 62160 | non- | TTC1918 | aspartate | ARA_LEAF | p-PcUBI | GC | 50 | 141 |
| | targeted | | | | | | | |
| 62160 | non- | TTC1918 | serine | ARA_LEAF | p-PcUBI | GC | 24 | 44 |
| | targeted | | | | | | | |
| 62244 | non- | XM_473199 | aspartate | ARA_LEAF | p-PcUBI | GC | 53 | 91 |
| | targeted | | | | | | | |
| 62244 | non- | XM_473199 | phenyl- | ARA_LEAF | p-PcUBI | GC | 31 | 44 |
| | targeted | | alanine | | | | | |
| 62244 | non- | XM_473199 | tyrosine | ARA_LEAF | p-PcUBI | GC | 35 | 47 |
| | targeted | | | | | | | |
| 62244 | non- | XM_473199 | 5-oxo- | ARA_LEAF | p-PcUBI | GC | 32 | 86 |
| | targeted | | proline | | | | | |
| 62524 | non- | Ybl021c | glycine | ARA_LEAF | Big35S | GC | 42 | 325 |
| | targeted | | | | | | | |
| 62524 | non- | Ybl021c | phenyl- | ARA_LEAF | Big35S | LC | 31 | 40 |
| | targeted | | alanine | | | | | |
| 62717 | non- | Ybr160w | aspartate | ARA_LEAF | Big35S | GC | 53 | 86 |
| | targeted | | | | | | | |
| 62717 | non- | Ybr160w | tyrosine | ARA_LEAF | Big35S | GC | 64 | 100 |
| | targeted | | | | | | | |
| 88879 | plastidic | Ybr249c | phenyl- | ARA_LEAF | p-Super | GC | 210 | 5005 |
| | | | alanine | | | | | |
| 88879 | plastidic | Ybr249c | tyrosine | ARA_LEAF | p-Super | GC | 159 | 2254 |
| 89317 | non- | Ybr296c | 5-oxo- | ARA_LEAF | p-PcUBI | GC | 33 | 51 |
| | targeted | | proline | | | | | |
| 89395 | plastidic | Ycl064c | alanine | ARA_LEAF | p-Super | GC | 23 | 51 |
| 89395 | plastidic | Ycl064c | glycine | ARA_LEAF | p-Super | GC | 36 | 106 |
| 14275 | plastidic | Ydl159w | aspartate | ARA_LEAF | p-Super | GC | 70 | 87 |
| 89483 | plastidic | Ydl188c | homo- | ARA_LEAF | p-Super | GC | 36 | 75 |
| | | | serine | | | | | |
| 89483 | plastidic | Ydl188c | phenyl- | ARA_LEAF | p-Super | GC | 28 | 41 |
| | | | alanine | | | | | |
| 89936 | plastidic | Ydl209c | 5-oxo- | ARA_LEAF | p-Super | GC | 46 | 97 |
| | | | proline | | | | | |
| 89973 | plastidic | Ydr007w | phenyl- | ARA_LEAF | p-Super | GC | 31 | 50 |
| | | | alanine | | | | | |
| 63544 | plastidic | Ydr046c | tyrosine | ARA_LEAF | p-Super | LC | 39 | 95 |
| 90103 | plastidic | Ydr183w | serine | ARA_LEAF | p-Super | GC | 25 | 80 |
| 90159 | plastidic | Ydr273w | aspartate | ARA_LEAF | p-Super | GC | 23 | 52 |
| 90159 | plastidic | Ydr273w | citrulline | ARA_LEAF | p-Super | LC | 34 | 36 |
| 90165 | non- | Ydr507c | aspartate | ARA_LEAF | Big35S | GC | 64 | 78 |
| | targeted | | | | | | | |
| 63745 | plastidic | Yer014w | asparagine | ARA_LEAF | p-PcUBI | GC | 46 | 159 |
| 63745 | plastidic | Yer014w | aspartate | ARA_LEAF | p-PcUBI | GC | 48 | 84 |
| 63745 | plastidic | Yer014w | serine | ARA_LEAF | p-PcUBI | GC | 25 | 82 |
| 63803 | plastidic | Yer106w | citrulline | ARA_LEAF | p-Super | LC | 34 | 85 |
| 63807 | non- | Yfl016c | serine | ARA_LEAF | Big35S | GC | 23 | 57 |
| | targeted | | | | | | | |
| 63807 | non- | Yfl016c | tyrosine | ARA_LEAF | Big35S | LC | 44 | 182 |
| | targeted | | | | | | | |
| 64144 | non- | Yfl019c | aspartate | ARA_LEAF | Big35S | GC | 103 | 305 |
| | targeted | | | | | | | |
| 64144 | non- | Yfl019c | phenyl- | ARA_LEAF | Big35S | GC | 34 | 185 |
| | targeted | | alanine | | | | | |
| 90189 | plastidic | Yfl030w | alanine | ARA_LEAF | p-Super | GC | 29 | 33 |
| 90189 | plastidic | Yfl030w | glycine | ARA_LEAF | p-Super | GC | 34 | 99 |
| 64148 | plastidic | Yfl054c | phenylalanine | ARA_LEAF | p-Super | GC | 36 | 39 |
| 64148 | plastidic | Yfl054c | serine | ARA_LEAF | p-Super | GC | 23 | 51 |
| 90278 | plastidic | Ygl096w | 5-oxo- | ARA_LEAF | p-Super | GC | 34 | 133 |
| | | | proline | | | | | |
| 64177 | non- | Ygl237c | tyrosine | ARA_LEAF | Big35S | LC | 45 | 166 |
| | targeted | | | | | | | |
| 64315 | plastidic | Ygr221c | aspartate | ARA_LEAF | p-Super | GC | 50 | 98 |
| 64315 | plastidic | Ygr221c | 5-oxo- | ARA_LEAF | p-Super | GC | 40 | 44 |
| | | | proline | | | | | |
| 14715 | plastidic | Yhl013c | phenyl- | ARA_LEAF | p-Super | GC | 35 | 410 |
| | | | alanine | | | | | |
| 90284 | non- | Yhr006w | Phenyl- | ARA_LEAF | Big35S | GC | 32 | 67 |
| | targeted | | alanine | | | | | |
| 90284 | non- | Yhr006w | tyrosine | ARA_LEAF | Big35S | GC | 39 | 129 |
| | targeted | | | | | | | |
| 64546 | plastidic | Yhr207c | aspartate | ARA_LEAF | p-Super | GC | 52 | 81 |
| 64563 | plastidic | Yil074c | citrulline | ARA_LEAF | p-Super | LC | 37 | 81 |
| 64563 | plastidic | Yil074c | glycine | ARA_LEAF | p-Super | GC | 57 | 94 |
| 90306 | plastidic | Yir038c | aspartate | ARA_LEAF | p-Super | GC | 49 | 72 |
| 90306 | plastidic | Yir038c | citrulline | ARA_LEAF | p-Super | LC | 35 | 43 |
| 64964 | plastidic | Yjl073w | aspartate | ARA_LEAF | p-Super | GC | 52 | 62 |
| 14843 | non- | Yjr130c | phenyl- | ARA_LEAF | Big35S | GC | 26 | 48 |
| | targeted | | alanine | | | | | |
| 90409 | plastidic | Yjr148w | citrulline | ARA_LEAF | p-Super | LC | 31 | 67 |
| 66274 | non- | Yjr153w | aspartate | ARA_LEAF | p-PcUBI | GC | 59 | 149 |
| | targeted | | | | | | | |
| 66274 | non- | Yjr153w | phenyl- | ARA_LEAF | p-PcUBI | LC | 34 | 51 |
| | targeted | | alanine | | | | | |
| 90602 | plastidic | YKL038W | alanine | ARA_LEAF | p-PcUBI | GC | 20 | 45 |
| 90602 | plastidic | YKL038W | aspartate | ARA_LEAF | p-PcUBI | GC | 59 | 125 |
| 90602 | plastidic | YKL038W | serine | ARA_LEAF | p-PcUBI | GC | 27 | 57 |
| 90615 | non- | Ylr027c | glycine | ARA_LEAF | Big35S | GC | 41 | 69 |
| | targeted | | | | | | | |
| 91045 | plastidic | Ylr043c | serine | ARA_LEAF | p-Super | GC | 29 | 110 |
| 91045 | plastidic | Ylr043c | tyrosine | ARA_LEAF | p-Super | LC | 40 | 94 |
| 91659 | plastidic | Yml008c | alanine | ARA_LEAF | p-PcUBI | GC | 21 | 52 |
| 91659 | plastidic | Yml008c | glycine | ARA_LEAF | p-PcUBI | GC | 40 | 96 |
| 91659 | plastidic | Yml008c | homo- | ARA_LEAF | p-PcUBI | GC | 30 | 46 |
| | | | serine | | | | | |
| 91659 | plastidic | Yml008c | 5-oxo- | ARA_LEAF | p-PcUBI | GC | 32 | 56 |
| | | | proline | | | | | |
| 91781 | plastidic | Yml083c | alanine | ARA_LEAF | p-Super | GC | 22 | 31 |
| 91785 | plastidic | Yml123c | phenyl- | ARA_LEAF | p-Super | LC | 32 | 57 |
| | | | alanine | | | | | |
| 91785 | plastidic | Yml123c | tyrosine | ARA_LEAF | p-Super | LC | 44 | 66 |
| 91958 | plastidic | Ymr013c | tyrosine | ARA_LEAF | p-Super | LC | 41 | 93 |
| 91973 | plastidic | Ymr120c | glycine | ARA_LEAF | p-Super | GC | 37 | 39 |
| 92274 | plastidic | Ymr150c | phenyl- | ARA_LEAF | p-Super | GC | 25 | 52 |
| | | | alanine | | | | | |
| 92325 | plastidic | Ynl086w | homo- | ARA_LEAF | p-Super | GC | 24 | 37 |
| | | | serine | | | | | |
| 92339 | plastidic | Ynl101w | tyrosine | ARA_LEAF | p-Super | GC | 33 | 43 |
| 92391 | plastidic | Ynl277w | homo- | ARA_LEAF | p-PcUBI | GC | 214 | 1243 |
| | | | serine | | | | | |
| 92510 | plastidic | Yol130w | alanine | ARA_LEAF | p-Super | GC | 21 | 33 |
| 15179 | plastidic | Yol160w | aspartate | ARA_LEAF | p-Super | GC | 57 | 62 |
| 67968 | non- | Yor233w | citrulline | ARA_LEAF | Big35S | LC | 36 | 92 |
| | targeted | | | | | | | |
| 92540 | plastidic | Yor244w | phenyl- | ARA_LEAF | p-Super | GC | 44 | 423 |
| | | | alanine | | | | | |
| 92540 | plastidic | Yor244w | tyrosine | ARA_LEAF | p-Super | GC | 53 | 85 |
| 92616 | non- | Yor245c | homo- | ARA_LEAF | Big35S | GC | 29 | 61 |
| | targeted | | serine | | | | | |
| 92616 | non- | Yor245c | serine | ARA_LEAF | Big35S | GC | 24 | 82 |
| | targeted | | | | | | | |
| 92616 | non- | Yor245c | 5-oxo- | ARA_LEAF | Big35S | GC | 35 | 145 |
| | targeted | | proline | | | | | |
| 92668 | non- | Ypl028w | glycine | ARA_LEAF | Big35S | GC | 43 | 82 |
| | targeted | | | | | | | |
| 93091 | non- | Ypr001w | serine | ARA_LEAF | Big35S | GC | 23 | 31 |
| | targeted | | | | | | | |
| 93218 | non- | Ypr138c | aspartate | ARA_SEED_2 | p-USP | GC | 42 | 217 |
| | targeted | | | | | | | |
| 68413 | non- | Zm_4842_BE510522 | phenyl- | ARA_LEAF | p-PcUBI | GC | 29 | 242 |
| | targeted | | alanine | | | | | |
| 68413 | non- | Zm_4842_BE510522 | serine | ARA_LEAF | p-PcUBI | GC | 28 | 95 |
| | targeted | | | | | | | |
| 68413 | non- | Zm_4842_BE510522 | tyrosine | ARA_LEAF | p-PcUBI | GC | 67 | 154 |
| | targeted | | | | | | | |
| 68413 | non- | Zm_4842_BE510522 | 5-oxo- | ARA_LEAF | p-PcUBI | GC | 33 | 173 |
| | targeted | | proline | | | | | |

Column 1 shows the SEQ ID NO, Column 2 shows the expression type (targeted or non-targeted), Column 3 shows the “gene name” (locus), Column 4 shows the metabolite analyzed, Column 5 indicates the A. thaliana source tissue analyzed, Column 6 indicates the used promoter for expression, Column 7 indicates the analytical method. Columns 8 and 9 show the minumum and the maximum increase of the analyzed metabolite (in percent) in comparison to the wild type (=ratio_by_weight, given as percent increase).

When the analyses were repeated independently, all results proved to be significant.

[0505.1.1.3] to [0515.1.1.3] for the disclosure of these paragraphs see [0505.1.1.1] to [0515.1.1.1] above.

In a further embodiment, the present invention relates in paragraphs

[0000.1.1.4] to [0514.1.4.4].to a further process for the production of the fine chemical threonine as defined below and corresponding embodiments as described herein as follows.

[0001.1.1.4] to [0006.1.1.4] for the disclosure of these paragraphs see [0001.1.1.1] to [0006.1.1.1] above.

Following the approach of deregulating specific enzymes in the amino acid biosynthetic pathway an increase of the levels of free threonine is disclosed in U.S. Pat. No. 5,942,660 which is achieved by overexpression of either a wild-type or deregulated aspartate kinase, homoserine dehydrogenase or threonine synthase.

for the disclosure of this paragraph see [0008.1.1.1] above.

As described above, the essential amino acids are necessary for humans and many mammals, for example for livestock. Threonine is an important constituent in many body proteins and is necessary for the formation of tooth enamel protein, collagen and elastin, which both needed for healthy skin and wound healing. It is a precursor to the amino acids glycine and serine. It acts as a lipotropic in controlling fat build-up in the liver. Threonine is an immune stimulant because it promotes thymus growth and activity. It is a component of digestive enzymes and immune secretions from the gut, particularly mucins. It has been used as a supplement to help alleviate anxiety and some cases of depression. In animal production, as an important essential amino acid, threonine is normally the second limiting amino acid for pigs and the third limiting amino acid for chicken (Gallus gallus f. domestica, e.g. laying hen or broiler).

[0010.1.1.4] to [0011.1.1.4] for the disclosure of these paragraphs see [0010.1.1.1] to [0011.1.1.1] above.

It is an object of the present invention to develop an inexpensive process for the synthesis of threonine, especially L-threonine. Threonine is with methionine and lysine (depending on the organism) one of the amino acids, which are most frequently limiting.

for the disclosure of this paragraph see [0013.1.1.1] above

Accordingly, in a first embodiment, the invention relates to a process for the production of threonine, or, in other words, of the “fine chemical” or “fine chemical of the invention”.

The terms “fine chemical of the invention”, “fine chemical” or “the fine chemical” are used herein equally and relate in context of the paragraphs or sections [0014.1.4.4] to [0514.1.1.4] essentially to the metabolite or the metabolites indicated in column 7, application no. 4 of Tables I, II and IV in the respective line.

Further, the term “in context of any of the paragraphs [0014.1.4.4] to [0514.1.1.4]” as used herein means that for any of said paragraphs [0014.1.4.4] to [0514.1.1.4] the term “the fine chemical” is understood to follow the definition of paragraphs or sections [0014.1.4.4] and [0015.1.4.4], independently whether it refers to any other paragraph or not and whether the reference recites the term “fine chemical” in an other context.

Thus, in cases where one or more paragraphs or sections are incorporated by reference into any of the present paragraphs [0014.1.4.4] to [0514.1.1.4], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to the definition of paragraph [0014.1.4.4] and [0015.1.4.4].

Accordingly, in one embodiment, the term “fine chemical of the invention”, “fine chemical” or “the fine chemical” as used herein (alone or in combination, like in FCRP) means “threonine” in context of the nucleic acid or polypeptide sequences listed in the respective same line of any one of Tables I to IV of application no. 4 and indicating in column 7 the metabolite “threonine”. In one embodiment, the term threonine or the term “fine chemical” mean in context of the paragraphs or sections [0014.1.4.4] to [0514.1.1.4] at least one chemical compound with an activity of the above mentioned threonine, respectively.

Accordingly, in one embodiment, the terms “fine chemical of the invention”, “fine chemical” or “the fine chemical” mean in context of any of the paragraphs [0014.1.4.4] to [0514.1.1.4] threonine, preferably the L-enantiomer of threonine, its salts, ester or amides in free form or bound form. In a preferred embodiment, the term “the fine chemical” means threonine or its salts, in free form or bound form. In a preferred embodiment, the term “the fine chemical” means the L-enantiomer of threonine. On the other hand in case “threonine” is stated it means threonine itself, its salts, ester or amides in free form or bound form, preferably the L-enantiomer of threonine, its salts, ester or amides in free form or bound to proteins. In a preferred embodiment “threonine” means the Lenantiomer of threonine in free form. In another preferred embodiment “threonine” means the Lenantiomer of threonine in bound form.

Further, the term “in context of any of the paragraphs [0014.1.4.4] to [0514.1.1.4]” as used herein means that for any of said paragraphs [0014.1.4.4] to [0514.1.4.4] the term “the fine chemical” is understood to follow the definition of section [0014.1.4.4] or section [0015.1.4.4], independently whether it refers to any other paragraph or not and whether the reference recites the term “fine chemical” in an other context.

Thus, in cases where one or more paragraphs or sections are incorporated by reference into any of the present paragraphs [0014.1.4.4] to [0514.1.1.4], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to this definition of this paragraph [0015.1.4.4].

Further, the term “fine chemicals” as used herein relates to compositions comprising said fine chemical(s), i.e. comprising threonine, respectively.

Accordingly, the present invention relates to a process for the production of threonine, which comprises
(a) increasing or generating one or more activities selected from the group consisting of 47266012-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of 49747384_SOYBEAN-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of glucose-6-phosphate 1-dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of At1g09680-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of bifunctional aspartokinase/homoserine dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of eukaryotic translation initiation factor 5 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of CCAAT-binding transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of glycosyl transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of monthiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of At4g32480-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of gibberellin 20-oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of aspartate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of At5g16650-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of thioredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of zinc finger protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of CDP-diacylglycerol-serine O-phosphatidyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of malic enzyme in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of secindependent protein translocase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of betahydroxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of elongation factor Tu in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of acyl-CoA synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of 2-oxoglutarate dehydrogenase E1 subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of permease protein of peptide ABC transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of GDP-mannose dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of enoyl-CoA hydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of homoserine dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of glutamate-ammonia-ligase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of peptidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of Chaperone protein CIpB in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of transcriptional regulator protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of AX653549-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of threonine synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of acetolactate synthase small subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of dihydrolipoamide acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of glucose dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of serine protease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of acyl-CoA dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of beta-galactosidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of 2,3-dihydroxyphenylpropionate 1,2-dioxygenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of ATP-binding component of a transport system in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of membrane transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of isochorismate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of sensor histidine kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of succinyl-CoA synthetase beta chain in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of major facilitator superfamily transporter protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of phosphoanhydride phosphorylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of b1003-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of b1108-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of restriction alleviation protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of b1445-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of NADP-dependent malic enzyme in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of b1522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of b1670-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of b1930-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of b2360-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of D-serine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of b2399-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of b2513-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of b2613-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of murein transglycosylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of b2739-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of 2-C-methyl-D-erythritol 4-phosphate cytidylyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of b2812-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of b2812-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of D-3-phosphoglycerate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of Lasparaginase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of glycoprotease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of b3083-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of glycogen synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of b3442-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of valine-pyruvate transaminase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of b3646-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of ATP synthase subunit beta in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of dihydroxyacid dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of ketol-acid reductoisomerase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of enterobacterial common antigen polymerase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of DNA helicase II in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of b3989-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of aspartate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of b4029-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of b4056-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of b4250-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of heat shock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of polyphosphate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of branched chain amino acid ABC transporter ATP-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of uroporphyrin-III C-methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of sll0418-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of oxireductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of malate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of phosphoribosylaminoimidazole carboxylase catalytic subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of glycogen synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of glycogen (starch) synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of nitrate/nitrite transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of carbamoyl-phosphate synthase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of glutathione S-transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of ABC metal ion transporter substrate-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of coproporphyrinogen III oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of ferredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of slr0383-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of bifunctional purine biosynthesis protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of aspartate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of geranylgeranyl pyrophosphate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of argininosuccinate lyase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of photosystem II protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of cell division protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of branched-chain amino acid ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of TTC0881-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of metal-dependent hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of homocitrate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of XM_{—}473199-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of cell division control protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of pyruvate carboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of ycl027w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of coproporphyrinogen III oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of ydr183w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of hexose transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of YEL030C-A-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of hexose transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of yfl054c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of ygl237c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of yhr131c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of (DL)-glycerol-3-phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of 3-phosphoglycerate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of glutathione S-transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of DnaJ-like chaperone in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of YJL127W-A-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of hexose transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of homoserine dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of branched chain aminotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of YKL038W-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of phosphoenolpyruvate carboxykinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of aspartate aminotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of mitochondrial dicarboxylate transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of phosphate transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of histone acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of cyclin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating one or more activities selected from the group consisting of Zm_{—}4842_BE510522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

An embodiment of the present invention relates to a process for the production of threonine, which comprises
(a1) increasing or generating the activity of a polypeptide comprising a polypeptide as depicted in a respective line in column 5 or 8 of Table II, application no. 4, or a homolog or a fragment thereof, a consensus sequence or at least one polypeptide motif as depicted in the respective line in column 8 of Table IV, application no. 4; or
(a2) increasing or generating the activity of an expression product of one or more nucleic acid molecule(s) comprising a polynucleotide as depicted in the respective line in column 5 or 8 of Table I, application no. 4, preferably the coding region thereof, or a homolog or a fragment thereof; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of threonine or a composition comprising threonine in said non-human organism or in the culture medium surrounding said non-human organism.

Accordingly, the increase or generation of one or more said activities is for example conferred by one or more expression products of said nucleic acid molecule, e.g. proteins. Accordingly, in the present invention described above, the increase or generation of one or more said activities is for example conferred by one or more protein(s) each comprising a polypeptide selected from the group as depicted in Table II, application no. 4, column 5 and 8, or a homolog or a fragment thereof.

The process of the invention comprises in one embodiment the following steps:
(i) increasing or generating of the expression of; and/or
(ii) increasing or generating the expression of an expression product of; and/or
(iii) increasing or generating one or more activities of an expression product encoded by;
   at least one nucleic acid molecule (in the following “Fine Chemical Related Protein (FCRP)”-encoding gene or “FCRP”-gene) comprising a nucleic acid molecule selected from the group consisting of:
(a) a nucleic acid molecule encoding the polypeptide shown in column 5 or 8 of Table II, preferably Table II B, application no. 4, or a homolog or a fragment thereof;
(b) a nucleic acid molecule shown in column 5 or 8 of Table I, preferably Table I B, application no. 4, or a homolog or a fragment thereof (preferably the coding region thereof);
(c) a nucleic acid molecule, which, as a result of the degeneracy of the genetic code, can be derived from a polypeptide sequence depicted in column 5 or 8 of Table II, preferably Table II B, application no. 4;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 4, or the coding region thereof;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 4;
(i) a nucleic acid molecule encoding a polypeptide having the activity represented by a respective protein as depicted in column 5 of Table II, application no. 4;
(j) a nucleic acid molecule which comprises a polynucleotide, which is obtained by amplifying a cDNA library or a genomic library using the primers as depicted in column 8 of Table III, application no. 4; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

The term “increasing or generating of the expression of” may also be interchanged by the term “overexpressing”.

In a preferred embodiment thereof said nucleic acid molecule encodes a polypeptide which has the activity of the polypeptide represented by a protein comprising a polypeptide as depicted in the corresponding hit in column 5 of Table II, application no. 4.

In another preferred embodiment thereof said nucleic acid molecule confers a generation or an increase of the respective fine chemical in a non-human organism or a part thereof as compared to a corresponding wild-type non-human organism or part thereof.

In another preferred embodiment thereof said nucleic acid molecule encodes a polypeptide which has the activity of the polypeptide represented by a protein as depicted in the corresponding hit in column 5 of Table II, application no. 4, and confers a generation or an increase of the respective fine chemical in a non-human organism or a part thereof as compared to a corresponding wild-type non-human organism or part thereof.

Accordingly, the genes of the present invention or used in accordance with the present invention, which respectively encode a protein having an activity of (DL)-glycerol-3-phosphatase, 2,3-dihydroxyphenylpropionate 1,2-dioxygenase, 2-C-methyl-D-erythritol 4-phosphate cytidylyltransferase, 2-oxoglutarate dehydrogenase E1 subunit, 3-phosphoglycerate dehydrogenase, 47266012-protein, 49747384_SOYBEAN-protein, ABC metal ion transporter substrate-binding protein, ABC transporter permease protein, acetolactate synthase small subunit, acetyltransferase, acyl-CoA dehydrogenase, acyl-CoA synthase, argininosuccinate lyase, aspartate aminotransferase, aspartate kinase, At1g09680-protein, At4g32480-protein, At5g16650-protein, ATP synthase subunit beta, ATP-binding component of a transport system, AX653549-protein, b1003-protein, b1108-protein, b1445-protein, b1522-protein, b1670-protein, b1930-protein, b2360-protein, b2399-protein, b2513-protein, b2613-protein, b2739-protein, b2812-protein, b3083-protein, b3442-protein, b3646-protein, b3989-protein, b4029-protein, b4056-protein, b4250-protein, beta-galactosidase, beta-hydroxylase, bifunctional aspartokinase/homoserine dehydrogenase, bifunctional purine biosynthesis protein, branched chain amino acid ABC transporter ATP-binding protein, branched chain aminotransferase, branched-chain amino acid ABC transporter permease protein, calcium-dependent protein kinase, carbamoyl-phosphate synthase subunit, CCAAT-binding transcription factor, CDP-d iacylglycerolserine O-phosphatidyltransferase, cell division control protein, cell division protein, Chaperone protein CIpB, coproporphyrinogen III oxidase, cyclin, cyclin D , D-3-phosphoglycerate dehydrogenase, dihydrolipoamide acetyltransferase, dihydroxyacid dehydratase, DNA helicase II, DnaJ-like chaperone, D-serine dehydratase, elongation factor Tu, enoyl-CoA hydratase, enterobacterial common antigen polymerase, eukaryotic translation initiation factor 5, ferredoxin, GDP-mannose dehydrogenase, geranylgeranyl pyrophosphate synthase, gibberellin 20-oxidase, glucose dehydrogenase, glucose-6-phosphate 1-dehydrogenase, glutamate-ammonia-ligase, glutaredoxin, glutathione S-transferase, glycogen (starch) synthase, glycogen synthase, glycoprotease, glycosyl transferase, heat shock protein, heat shock transcription factor, hexose transporter, histone acetyltransferase, homocitrate synthase, homoserine dehydrogenase, hydrolase, isochorismate synthase, ketol-acid reductoisomerase, kinase, L-asparaginase, major facilitator superfamily transporter protein, malate dehydrogenase, malic enzyme, membrane transport protein, metal-dependent hydrolase, methyltransferase, mitochondrial dicarboxylate transporter, monothiol glutaredoxin, monthiol glutaredoxin, murein transglycosylase, NADP-dependent malic enzyme, nitrate/nitrite transport protein, oxireductase, peptidase, permease protein of peptide ABC transporter, phosphate transporter, phosphoanhydride phosphorylase, phosphoenolpyruvate carboxykinase, phosphoribosylaminoimidazole carboxylase catalytic subunit, photosystem II protein, polyphosphate kinase, protein kinase, protein phosphatase, pyruvate carboxylase, pyruvate kinase, restriction alleviation protein, sec-independent protein translocase, sensor histidine kinase, serine protease, sll0418-protein, slr0383-protein, succinyl-CoA synthetase beta chain, thioredoxin, threonine synthase, transcription factor, transcriptional regulator, transcriptional regulator protein, transport protein, TTC0881-protein, uroporphyrin-III C-methyltransferase, valine-pyruvate transaminase, XM_{—}473199-protein, ycl027w-protein, ydr183w-protein, YEL030C-A-protein, yfl054c-protein, ygl237c-protein, yhr131c-protein, YJL127W-A-protein, YKL038W-protein, zinc finger protein, or Zm_{—}4842_BE510522-protein, which respectively encode a protein comprising a polypeptide encoded by a nucleic acid sequence as shown in Table I, application no. 4, column 5 or 8, (preferably the coding region thereof), or a homolog or a fragment thereof, which respectively encode a protein comprising a polypeptide as depicted in Table II, application no. 4, column 5 or 8, or a homolg or a fragment thereof, and/or which respectively can be amplified with the primer set shown in Table III, application no. 4, column 8, are also referred to as “FCRP genes”.

Proteins or polypeptides encoded by “FCRP-genes” are referred to as “Fine Chemical Related Proteins” or “FCRP”. For the purposes of the description of the present invention, the respective protein having an activity of (DL)-glycerol-3-phosphatase, 2,3-dihydroxyphenylpropionate 1,2-dioxygenase, 2-C-methyl-D-erythritol 4-phosphate cytidylyltransferase, 2-oxoglutarate dehydrogenase E1 subunit, 3-phosphoglycerate dehydrogenase, 47266012-protein, 49747384_SOYBEAN-protein, ABC metal ion transporter substrate-binding protein, ABC transporter permease protein, acetolactate synthase small subunit, acetyltransferase, acyl-CoA dehydrogenase, acyl-CoA synthase, argininosuccinate lyase, aspartate aminotransferase, aspartate kinase, At1g09680-protein, At4g32480-protein, At5g16650-protein, ATP synthase subunit beta, ATP-binding component of a transport system, AX653549-protein, b1003-protein, b1108-protein, b1445-protein, b1522-protein, b1670-protein, b1930-protein, b2360-protein, b2399-protein, b2513-protein, b2613-protein, b2739-protein, b2812-protein, b3083-protein, b3442-protein, b3646-protein, b3989-protein, b4029-protein, b4056-protein, b4250-protein, beta-galactosidase, beta-hydroxylase, bifunctional aspartokinase/homoserine dehydrogenase, bifunctional purine biosynthesis protein, branched chain amino acid ABC transporter ATP-binding protein, branched chain aminotransferase, branched-chain amino acid ABC transporter permease protein, calcium-dependent protein kinase, carbamoyl-phosphate synthase subunit, CCAAT-binding transcription factor, CDP-diacylglycerol-serine O-phosphatidyltransferase, cell division control protein, cell division protein, Chaperone protein CIpB, coproporphyrinogen III oxidase, cyclin, cyclin D , D-3-phosphoglycerate dehydrogenase, dihydrolipoamide acetyltransferase, dihydroxyacid dehydratase, DNA helicase II, DnaJ-like chaperone, D-serine dehydratase, elongation factor Tu, enoyl-CoA hydratase, enterobacterial common antigen polymerase, eukaryotic translation initiation factor 5, ferredoxin, GDP-mannose dehydrogenase, geranylgeranyl pyrophosphate synthase, gibberellin 20-oxidase, glucose dehydrogenase, glucose-6-phosphate 1-dehydrogenase, glutamate-ammonia-ligase, glutaredoxin, glutathione S-transferase, glycogen (starch) synthase, glycogen synthase, glycoprotease, glycosyl transferase, heat shock protein, heat shock transcription factor, hexose transporter, histone acetyltransferase, homocitrate synthase, homoserine dehydrogenase, hydrolase, isochorismate synthase, ketol-acid reductoisomerase, kinase, L-asparaginase, major facilitator superfamily transporter protein, malate dehydrogenase, malic enzyme, membrane transport protein, metal-dependent hydrolase, methyltransferase, mitochondrial dicarboxylate transporter, monothiol glutaredoxin, monthiol glutaredoxin, murein transglycosylase, NADP-dependent malic enzyme, nitrate/nitrite transport protein, oxireductase, peptidase, permease protein of peptide ABC transporter, phosphate transporter, phosphoanhydride phosphorylase, phosphoenolpyruvate carboxykinase, phosphoribosylaminoimidazole carboxylase catalytic subunit, photosystem II protein, polyphosphate kinase, protein kinase, protein phosphatase, pyruvate carboxylase, pyruvate kinase, restriction alleviation protein, sec-independent protein translocase, sensor histidine kinase, serine protease, sll0418-protein, slr0383-protein, succinyl-CoA synthetase beta chain, thioredoxin, threonine synthase, transcription factor, transcriptional regulator, transcriptional regulator protein, transport protein, TTC0881-protein, uroporphyrin-III C-methyltransferase, valine-pyruvate transaminase, XM_{—}473199-protein, ycl027w-protein, ydr183w-protein, YEL030C-A-protein, yfl054c-protein, ygl237c-protein, yhr131c-protein, YJL127W-A-protein, YKL038W-protein, zinc finger protein, or Zm_{—}4842_BE510522-protein, the respective protein comprising a polypeptide encoded by one or more respective nucleic acid sequences as shown in Table I, application no. 4, column 5 or 8, (preferably the coding region thereof), or a homolog or fragment thereof, the respective protein comprising a respective polypeptide as depicted in Table II, application no. 4, column 5 or 8, or a homolog or fragment thereof, the respective protein comprising a sequence corresponding to the consensus sequence as shown in Table IV, application no. 4, column 8, and/or the respective protein comprising at least one polypeptide motif as shown in Table IV, application no. 4, column 8 is also referred to as Fine Chemical Related Protein” or “FCRP”.

Thus, in one embodiment, the present invention provides a process of the production of threonine, by increasing or generating one or more activities, especially selected from the group consisting of (DL)-glycerol-3-phosphatase, 2,3-dihydroxyphenylpropionate 1,2-dioxygenase, 2-C-methyl-D-erythritol 4-phosphate cytidylyltransferase, 2-oxoglutarate dehydrogenase E1 subunit, 3-phosphoglycerate dehydrogenase, 47266012-protein, 49747384_SOYBEAN -protein, ABC metal ion transporter substrate-binding protein, ABC transporter permease protein, acetolactate synthase small subunit, acetyltransferase, acyl-CoA dehydrogenase, acyl-CoA synthase, argininosuccinate lyase, aspartate aminotransferase, aspartate kinase, At1g09680-protein, At4g32480-protein, At5g16650-protein, ATP synthase subunit beta, ATP-binding component of a transport system, AX653549-protein, b1003-protein, b1108-protein, b1445-protein, b1522-protein, b1670-protein, b1930-protein, b2360-protein, b2399-protein, b2513-protein, b2613-protein, b2739-protein, b2812-protein, b3083-protein, b3442-protein, b3646-protein, b3989-protein, b4029-protein, b4056-protein, b4250-protein, beta-galactosidase, beta-hydroxylase, bifunctional aspartokinase/homoserine dehydrogenase, bifunctional purine biosynthesis protein, branched chain amino acid ABC transporter ATP-binding protein, branched chain aminotransferase, branched-chain amino acid ABC transporter permease protein, calcium-dependent protein kinase, carbamoyl-phosphate synthase subunit, CCAAT-binding transcription factor, CDP-diacylglycerol-serine O-phosphatidyltransferase, cell division control protein, cell division protein, Chaperone protein CIpB, coproporphyrinogen III oxidase, cyclin, cyclin D , D-3-phosphoglycerate dehydrogenase, dihydrolipoamide acetyltransferase, dihydroxyacid dehydratase, DNA helicase II, DnaJ-like chaperone, D-serine dehydratase, elongation factor Tu, enoyl-CoA hydratase, enterobacterial common antigen polymerase, eukaryotic translation initiation factor 5, ferredoxin, GDP-mannose dehydrogenase, geranylgeranyl pyrophosphate synthase, gibberellin 20-oxidase, glucose dehydrogenase, glucose-6-phosphate 1-dehydrogenase, glutamate-ammonia-ligase, glutaredoxin, glutathione S-transferase, glycogen (starch) synthase, glycogen synthase, glycoprotease, glycosyl transferase, heat shock protein, heat shock transcription factor, hexose transporter, histone acetyltransferase, homocitrate synthase, homoserine dehydrogenase, hydrolase, isochorismate synthase, ketol-acid reductoisomerase, kinase, L-asparaginase, major facilitator superfamily transporter protein, malate dehydrogenase, malic enzyme, membrane transport protein, metal-dependent hydrolase, methyltransferase, mitochondrial dicarboxylate transporter, monothiol glutaredoxin, monthiol glutaredoxin, murein transglycosylase, NADP-dependent malic enzyme, nitrate/nitrite transport protein, oxireductase, peptidase, permease protein of peptide ABC transporter, phosphate transporter, phosphoanhydride phosphorylase, phosphoenolpyruvate carboxykinase, phosphoribosylaminoimidazole carboxylase catalytic subunit, photosystem II protein, polyphosphate kinase, protein kinase, protein phosphatase, pyruvate carboxylase, pyruvate kinase, restriction alleviation protein, sec-independent protein translocase, sensor histidine kinase, serine protease, sll0418-protein, slr0383-protein, succinyl-CoA synthetase beta chain, thioredoxin, threonine synthase, transcription factor, transcriptional regulator, transcriptional regulator protein, transport protein, TTC0881-protein, uroporphyrin-111 C-methyltransferase, valine-pyruvate transaminase, XM_{—}473199-protein, ycl027w-protein, ydr183w-protein, YEL030C-A-protein, yfl054c-protein, ygl237c-protein, yhr131c-protein, YJL127W-A-protein, YKL038W-protein, zinc finger protein, and Zm_{—}4842_BE510522-protein, which is conferred by one or more FCRPs or the gene product of one or more FCRP-genes, for example by the gene product of a nucleic acid sequences comprising a polynucleotide selected from the group as shown in Table I, application no. 4, column 5 or 8, (preferably by the coding region thereof), or a homolog or a fragment thereof, e.g. or by one or more proteins each comprising a polypeptide encoded by one or more nucleic acid sequences selected from the group as shown in Table I, application no. 4, column 5 or 8, (preferably by the coding region thereof), or a homolog or a fragment thereof, or by one or more protein(s) each comprising a polypeptide selected from the group as depicted in Table II, application no. 4, column 5 and 8, or a homolog thereof, or a protein comprising a sequence corresponding to the consensus sequence or comprising at least one polypeptide motif as shown in Table IV, application no. 4, column 8.

for the disclosure of this paragraph see [0025.1.1.1] above.

In an embodiment, the process comprises increasing or generating the activity of one or more polypeptides having said activity, e.g. by generating or increasing the amount and/or specific activity in the cell or a compartment of a cell of one of more FCRP, especially selected from the group consisting of (DL)-glycerol-3-phosphatase, 2,3-dihydroxyphenylpropionate 1,2-dioxygenase, 2-C-methyl-D-erythritol 4-phosphate cytidylyltransferase, 2-oxoglutarate dehydrogenase E1 subunit, 3-phosphoglycerate dehydrogenase, 47266012-protein, 49747384_SOYBEAN-protein, ABC metal ion transporter substrate-binding protein, ABC transporter permease protein, acetolactate synthase small subunit, acetyltransferase, acyl-CoA dehydrogenase, acyl-CoA synthase, argininosuccinate lyase, aspartate aminotransferase, aspartate kinase, At1g09680-protein, At4g32480-protein, At5g16650-protein, ATP synthase subunit beta, ATP-binding component of a transport system, AX653549-protein, b1003-protein, b1108-protein, b1445-protein, b1522-protein, b1670-protein, b1930-protein, b2360-protein, b2399-protein, b2513-protein, b2613-protein, b2739-protein, b2812-protein, b3083-protein, b3442-protein, b3646-protein, b3989-protein, b4029-protein, b4056-protein, b4250-protein, beta-galactosidase, beta-hydroxylase, bifunctional aspartokinase/homoserine dehydrogenase, bifunctional purine biosynthesis protein, branched chain amino acid ABC transporter ATP-binding protein, branched chain aminotransferase, branched-chain amino acid ABC transporter permease protein, calcium-dependent protein kinase, carbamoyl-phosphate synthase subunit, CCAAT-binding transcription factor, CDP-diacylglycerolserine O-phosphatidyltransferase, cell division control protein, cell division protein, Chaperone protein CIpB, coproporphyrinogen III oxidase, cyclin, cyclin D , D-3-phosphoglycerate dehydrogenase, dihydrolipoamide acetyltransferase, dihydroxyacid dehydratase, DNA helicase II, DnaJ-like chaperone, D-serine dehydratase, elongation factor Tu, enoyl-CoA hydratase, enterobacterial common antigen polymerase, eukaryotic translation initiation factor 5, ferredoxin, GDP-mannose dehydrogenase, geranylgeranyl pyrophosphate synthase, gibberellin 20-oxidase, glucose dehydrogenase, glucose-6-phosphate 1-dehydrogenase, glutamate-ammonia-ligase, glutaredoxin, glutathione S-transferase, glycogen (starch) synthase, glycogen synthase, glycoprotease, glycosyl transferase, heat shock protein, heat shock transcription factor, hexose transporter, histone acetyltransferase, homocitrate synthase, homoserine dehydrogenase, hydrolase, isochorismate synthase, ketol-acid reductoisomerase, kinase, L-asparaginase, major facilitator superfamily transporter protein, malate dehydrogenase, malic enzyme, membrane transport protein, metal-dependent hydrolase, methyltransferase, mitochondrial dicarboxylate transporter, monothiol glutaredoxin, monthiol glutaredoxin, murein transglycosylase, NADP-dependent malic enzyme, nitrate/nitrite transport protein, oxireductase, peptidase, permease protein of peptide ABC transporter, phosphate transporter, phosphoanhydride phosphorylase, phosphoenolpyruvate carboxykinase, phosphoribosylaminoimidazole carboxylase catalytic subunit, photosystem II protein, polyphosphate kinase, protein kinase, protein phosphatase, pyruvate carboxylase, pyruvate kinase, restriction alleviation protein, sec-independent protein translocase, sensor histidine kinase, serine protease, sll0418-protein, slr0383-protein, succinyl-CoA synthetase beta chain, thioredoxin, threonine synthase, transcription factor, transcriptional regulator, transcriptional regulator protein, transport protein, TTC0881-protein, uroporphyrin-III C-methyltransferase, valine-pyruvate transaminase, XM_{—}473199-protein, ycl027w-protein, ydr183w-protein, YEL030C-A-protein, yfl054c-protein, ygl237c-protein, yhr131c-protein, YJL127W-A-protein, YKL038W-protein, zinc finger protein, and Zm_{—}4842_BE510522-protein, for example of the respective polypeptide as depicted in Table II, application no. 4, column 5 and 8, or a homolog or a fragment thereof, or the respective polypeptide comprising a sequence corresponding to the consensus sequences as shown in Table IV, application no. 4, column 8, or the respective polypeptide comprising at least one polypeptide motif as depicted in Table IV, application no. 4, column 8.

A further embodiment of the present invention relates to a process for the production of threonine, which comprises
(a) increasing or generating the activity of a 47266012-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a 49747384_SOYBEAN-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a glucose-6-phosphate 1-dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a At1g09680-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a bifunctional aspartokinase/homoserine dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a eukaryotic translation initiation factor 5 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a CCAAT-binding transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a glycosyl transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a monthiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a At4g32480-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a gibberellin 20-oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a aspartate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a At5g16650-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a thioredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a zinc finger protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a CDP-diacylglycerol-serine O-phosphatidyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a malic enzyme non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a sec-independent protein translocase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a beta-hydroxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a elongation factor Tu non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a acyl-CoA synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a 2-oxoglutarate dehydrogenase E1 subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a permease protein of peptide ABC transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a GDP-mannose dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a enoyl-CoA hydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a homoserine dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a glutamate-ammonia-ligase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a peptidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a Chaperone protein CIpB non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a transcriptional regulator protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a AX653549-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a threonine synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a acetolactate synthase small subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a dihydrolipoamide acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a glucose dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a serine protease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a acyl-CoA dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a beta-galactosidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a 2,3-dihydroxyphenylpropionate 1,2-dioxygenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a ATP-binding component of a transport system non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a membrane transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a isochorismate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a sensor histidine kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a succinyl-CoA synthetase beta chain non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a major facilitator superfamily transporter protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a phosphoanhydride phosphorylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a b1003-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a b1108-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a restriction alleviation protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a b1445-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a NADP-dependent malic enzyme non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a b1522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a b1670-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a b1930-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a b2360-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a D-serine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a b2399-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a b2513-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a b2613-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a murein transglycosylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a b2739-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a 2-C-methyl-D-erythritol 4-phosphate cytidylyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a b2812-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a b2812-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a D-3-phosphoglycerate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a L-asparaginase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a glycoprotease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a b3083-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a glycogen synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a b3442-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a valine-pyruvate transaminase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a b3646-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a ATP synthase subunit beta non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a dihydroxyacid dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a ketol-acid reductoisomerase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a enterobacterial common antigen polymerase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a DNA helicase II non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a b3989-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a aspartate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a b4029-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a b4056-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a b4250-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a heat shock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a polyphosphate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a branched chain amino acid ABC transporter ATP-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a uroporphyrin-III C-methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a sll0418-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a oxireductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a malate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a phosphoribosylaminoimidazole carboxylase catalytic subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a glycogen synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a glycogen (starch) synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a nitrate/nitrite transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a carbamoyl-phosphate synthase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a glutathione S-transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a ABC metal ion transporter substrate-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a coproporphyrinogen III oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a ferredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a slr0383-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a bifunctional purine biosynthesis protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a aspartate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a geranylgeranyl pyrophosphate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a argininosuccinate lyase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a photosystem II protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a cell division protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a branched-chain amino acid ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a TTC0881-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a metal-dependent hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a homocitrate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a XM_{—}473199-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a cell division control protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a pyruvate carboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a ycl027w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a coproporphyrinogen III oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a ydr183w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a hexose transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a YEL030C-A-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a hexose transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a yfl054c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a ygl237c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a yhr131c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a (DL)-glycerol-3-phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a 3-phosphoglycerate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a glutathione S-transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a DnaJ-like chaperone non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a YJL127W-A-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a hexose transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a homoserine dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a branched chain aminotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a YKL038W-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a phosphoenolpyruvate carboxykinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a aspartate aminotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a mitochondrial dicarboxylate transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a phosphate transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a histone acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a cyclin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a Zm_{—}4842_BE510522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating the activity of a polypeptide comprising a polypeptide as depicted in the respective line in column 5 or 8 of Table II, application no. 4, or a homolog or a fragment thereof, a consensus sequence or at least one polypeptide motif as depicted in the respective line in column 8 of Table IV, application no. 4; or increasing or generating the activity of an expression product of one or more nucleic acid molecule(s) comprising a polynucleotide as depicted in the respective line in column 5 or 8 of Table I, application no. 4, preferably the coding region thereof, or a homolog or a fragment thereof;

non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of threonine, or a composition comprising threonine in said non-human organism or in the culture medium surrounding said non-human organism.

A further embodiment of the present invention relates to a process for the production of threonine, which comprises
(a) increasing or generating one or more activities selected from the group consisting of 47266012-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of 49747384_SOYBEAN-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of glucose-6-phosphate 1-dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of At1g09680-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of bifunctional aspartokinase/homoserine dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of eukaryotic translation initiation factor 5 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of CCAAT-binding transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of glycosy) transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of monthiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of At4g32480-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of gibberellin 20-oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of aspartate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of At5g16650-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of thioredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of zinc finger protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of CDP-diacylglycerol-serine O-phosphatidyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of malic enzyme in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of sec-independent protein translocase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of beta-hydroxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of elongation factor Tu in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of acyl-CoA synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of 2-oxoglutarate dehydrogenase E1 subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of permease protein of peptide ABC transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of GDP-mannose dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of enoyl-CoA hydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of homoserine dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of glutamate-ammonia-ligase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of peptidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of Chaperone protein CIpB in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of transcriptional regulator protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of AX653549-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of threonine synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of acetolactate synthase small subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of dihydrolipoamide acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of glucose dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of serine protease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of acyl-CoA dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of betagalactosidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of 2,3-dihydroxyphenylpropionate 1,2-dioxygenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of ATP-binding component of a transport system in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of membrane transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of isochorismate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of sensor histidine kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of succinyl-CoA synthetase beta chain in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of major facilitator superfamily transporter protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of phosphoanhydride phosphorylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of b1003-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of b1108-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of restriction alleviation protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of b1445-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of NADP-dependent malic enzyme in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of b1522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of b1670-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of b1930-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of b2360-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of Dserine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of b2399-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of b2513-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of b2613-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of murein transglycosylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of b2739-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of 2-C-methyl-D-erythritol 4-phosphate cytidylyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of b2812-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of b2812-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of D-3-phosphoglycerate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of L-asparaginase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of glycoprotease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of b3083-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of glycogen synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of b3442-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of valine-pyruvate transaminase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of b3646-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of ATP synthase subunit beta in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of dihydroxyacid dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of ketol-acid reductoisomerase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of enterobacterial common antigen polymerase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of DNA helicase II in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of b3989-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of aspartate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of b4029-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of b4056-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of b4250-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of heat shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of polyphosphate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of branched chain amino acid ABC transporter ATP-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of uroporphyrin-III C-methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of sll0418-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of oxireductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of malate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of phosphoribosylaminoimidazole carboxylase catalytic subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of glycogen synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of glycogen (starch) synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of nitrate/nitrite transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of carbamoyl-phosphate synthase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of glutathione S-transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of ABC metal ion transporter substrate-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of coproporphyrinogen III oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of ferredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of slr0383-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of bifunctional purine biosynthesis protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of aspartate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of geranylgeranyl pyrophosphate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of argininosuccinate lyase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of photosystem II protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of cell division protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of branched-chain amino acid ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of TTC0881-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of metal-dependent hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of homocitrate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of XM_{—}473199-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of cell division control protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of pyruvate carboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of ycl027w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of coproporphyrinogen III oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of ydr183w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of hexose transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of YEL030C-A-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of hexose transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of yfl054c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of ygl237c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of yhr131c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of (DL)-glycerol-3-phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of 3-phosphoglycerate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of glutathione S-transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of DnaJ-like chaperone in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of YJL127W-A-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of hexose transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of homoserine dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of branched chain aminotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of YKL038W-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of phosphoenolpyruvate carboxykinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of aspartate aminotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of mitochondrial dicarboxylate transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of phosphate transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of histone acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of cyclin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a) increasing or generating one or more activities selected from the group consisting of Zm_{—}4842_BE510522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

A further embodiment of the present invention relates to a process for the production of threonine, which comprises
(a1) increasing or generating the activity of a polypeptide comprising a polypeptide as depicted in the respective line in column 5 or 8 of Table II, application no. 4, or a homolog or fragment thereof, a consensus sequence or at least one polypeptide motif as depicted in column 8 of Table IV, application no. 4; or
increasing or generating the activity of an expression product of one or more nucleic acid molecule(s) comprising a polynucleotide as depicted in the respective line in column 5 or 8 of Table I, application no. 4, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 4, or a homolog or a fragment thereof, a consensus sequence or at least one polypeptide motif as depicted in the respective line in column 8 of Table IV, application no. 4, which is joined to a transit peptide; or
increasing or generating the activity of an expression product of one or more nucleic acid molecule(s) comprising a polynucleotide as depicted in the respective line in column 5 or 8 of Table I, application no. 4, preferably the coding region thereof, or a homolog or a fragment thereof, which is joined to a nucleic acid sequence encoding an organelle localization sequence, preferably a plastid or a mitochondrion localization sequence, especially a plastid localization sequence;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 4, or a homolog or a fragment thereof, a consensus sequence or at least one polypeptide motif as depicted in the respective line in column 8 of Table IV, application no. 4; or
increasing or generating the activity of an expression product of one or more nucleic acid molecule(s) comprising a polynucleotide as depicted in the respective line in column 5 or 8 of Table I, application no. 4, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of threonine, or a composition comprising threonine in said non-human organism or in the culture medium surrounding said non-human organism.

Accordingly, the present invention relates to a process for the production of threonine, which comprises
(a) increasing or generating the activity of a 47266012-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a 49747384_SOYBEAN-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a glucose-6-phosphate 1-dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a At1g09680-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a bifunctional aspartokinase/homoserine dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a eukaryotic translation initiation factor 5 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a CCAAT-binding transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a glycosyl transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a monthiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a At4g32480-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a gibberellin 20-oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a aspartate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a At5g16650-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a thioredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a zinc finger protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a CDP-diacylglycerol-serine O-phosphatidyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a malic enzyme in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a sec-independent protein translocase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a beta-hydroxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a elongation factor Tu in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a acyl-CoA synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a 2-oxoglutarate dehydrogenase E1 subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a permease protein of peptide ABC transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a GDP-mannose dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a enoyl-CoA hydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a homoserine dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a glutamate-ammonia-ligase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a peptidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a Chaperone protein CIpB in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a transcriptional regulator protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a AX653549-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a threonine synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a acetolactate synthase small subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a dihydrolipoamide acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a glucose dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a serine protease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a acyl-CoA dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a beta-galactosidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a 2,3-dihydroxyphenylpropionate 1,2-dioxygenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a ATP-binding component of a transport system in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a membrane transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a isochorismate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a sensor histidine kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a succinyl-CoA synthetase beta chain in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a major facilitator superfamily transporter protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a phosphoanhydride phosphorylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a b1003-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a b1108-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a restriction alleviation protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a b1445-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a NADP-dependent malic enzyme in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a b1522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a b1670-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a b1930-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a b2360-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a D-serine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a b2399-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a b2513-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a b2613-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a murein transglycosylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a b2739-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a 2-C-methyl-D-erythritol 4-phosphate cytidylyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a b2812-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a b2812-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a D-3-phosphoglycerate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a L-asparaginase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a glycoprotease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a b3083-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a glycogen synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a b3442-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a valine-pyruvate transaminase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a b3646-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a ATP synthase subunit beta in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a dihydroxyacid dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a ketol-acid reductoisomerase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a enterobacterial common antigen polymerase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a DNA helicase II in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a b3989-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a aspartate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a b4029-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a b4056-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a b4250-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a heat shock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a polyphosphate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a branched chain amino acid ABC transporter ATP-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a uroporphyrin-III C-methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a sll0418-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a oxireductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a malate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a phosphoribosylaminoimidazole carboxylase catalytic subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a glycogen synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a glycogen (starch) synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a nitrate/nitrite transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a carbamoyl-phosphate synthase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a glutathione S-transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a ABC metal ion transporter substrate-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a coproporphyrinogen III oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a ferredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a slr0383-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a bifunctional purine biosynthesis protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a aspartate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a geranylgeranyl pyrophosphate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a argininosuccinate lyase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a photosystem II protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a cell division protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a branched-chain amino acid ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a TTC0881-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a metal-dependent hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a homocitrate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a XM_{—}473199-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a cell division control protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a pyruvate carboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a ycl027w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a coproporphyrinogen III oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a ydr183w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a hexose transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a YEL030C-A-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a hexose transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a yfl054c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a ygl237c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a yhr131c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a (DL)-glycerol-3-phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a 3-phosphoglycerate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a glutathione S-transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a DnaJ-like chaperone in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a YJL127W-A-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a hexose transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a homoserine dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a branched chain aminotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a YKL038W-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a phosphoenolpyruvate carboxykinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a aspartate aminotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a mitochondrial dicarboxylate transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a phosphate transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a histone acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a cyclin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a Zm_{—}4842_BE510522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Accordingly, the present invention relates to a process for the production of threonine, which comprises
(a) increasing or generating the activity of a polypeptide comprising a polypeptide as depicted in the respective line in column 5 or 8 of Table II, application no. 4, or a homolog or a fragment thereof, a consensus sequence or at least one polypeptide motif as depicted in the respective line in column 8 of Table IV, application no. 4; or
increasing or generating the activity of an expression product of one or more nucleic acid molecule(s) comprising a polynucleotide as depicted in the respective line in column 5 or 8 of Table I, application no. 4, preferably the coding region thereof, or a homolog or a fragment thereof;

in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of threonine, or a composition comprising threonine in said non-human organism or in the culture medium surrounding said non-human organism.

In a further embodiment the activity of the polypeptide comprising a polypeptide as depicted in the respective line in column 5 or 8 of Table II, application no. 4, or a homolog or a fragment thereof, a consensus sequence or at least one polypeptide motif as depicted in the respective line in column 8 of Table IV, application no. 4, is increased or generated non-targeted in the above-mentioned process in a microorganism or plant or a part thereof.

In a further embodiment said polypeptide has the activity of the respective polypeptide represented by a protein comprising a polypeptide as depicted in the respective line in column 5 of Table II, application no. 4.

In a further embodiment the activity of the expression product of one or more nucleic acid molecule(s) comprising a polynucleotide as depicted in the respective line in column 5 or 8 of Table I, application no. 4, preferably the coding region thereof, or a homolog or a fragment thereof, is increased or generated non-targeted in the above-mentioned process in a microorganism or plant or a part thereof.

In a further embodiment the activity of the polypeptide comprising a polypeptide as depicted in the respective line in column 5 or 8 of Table II, application no. 4, or a homolog or a fragment thereof, a consensus sequence or at least one polypeptide motif as depicted in the respective line in column 8 of Table IV, application no. 4, is increased or generated in the above-mentioned process in an organelle preferably in plastids or mitochondria, especially in plastids, of a microorganism or plant.

In a further embodiment said polypeptide has the activity of the respective polypeptide represented by a protein comprising a polypeptide as depicted in the respective line in column 5 of Table II, application no. 4.

In a further embodiment the activity of the expression product of one or more nucleic acid molecule(s) comprising a polynucleotide as depicted in the respective line in column 5 or 8 of Table I, application no. 4, preferably the coding region thereof, or a homolog or a fragment thereof, is increased or generated in the above-mentioned process in an organelle, preferably in plasids or mitochondria, especially in plastids, of a microorganism or plant.

In a further embodiment the activity of the polypeptide comprising a polypeptide as depicted in the respective line in column 5 or 8 of Table II, application no. 4, or a homolog or a fragment thereof, a consensus sequence or at least one polypeptide motif as depicted in the respective line in column 8 of Table IV, application no. 4, is increased or generated in the above-mentioned process in the cyctosol of a cell, of a microorganism or plant.

In a further embodiment said polypeptide has the activity of the respective polypeptide represented by a protein comprising a polypeptide as depicted in the respective line in column 5 of Table II, application no. 4.

In a further embodiment the activity of the expression product of one or more nucleic acid molecule(s) comprising a polynucleotide as depicted in the respective line in column 5 or 8 of Table I, application no. 4, preferably the coding region thereof, or a homolog or a fragment thereof, is increased or generated in the above-mentioned process in the cytosol of a cell, of a microorganism or plant.

[0039.1.1.4] to [0066.1.1.4] for the disclosure of these paragraphs see [0039.1.1.1] to [0066.1.1.1 ]above.

As mentioned above the nucleic acid sequences coding for the proteins as shown in Table II, application no. 4, column 5 or 8, or homologs or fragments thereof, are joined to a nucleic acid sequence encoding a plastidic transit peptide, e.g., if for the nucleic acid molecule in Table I, column 6 the term “plastidic” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in Table II, application no. 4, column 5 or 8, or homologs or fragments thereof, e.g., if for the nucleic acid molecule in Table I, column 6 the term “plastidic” is indicated.

[0068.1.1.4] to [0072.1.1.4] for the disclosure of these paragraphs see [0068.1.1.1] to [0072.1.1.1] above.

As mentioned above the nucleic acid sequences coding for the proteins as shown in the respective line in Table II, application no. 4, column 5 or 8, or homologs or fragments thereof, are joined to a nucleic acid sequence encoding a mitochondric transit peptide, e.g., if for the respective nucleic acid molecule in Table I, column 6 the term “mitochondrial” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in the respective line in Table II, application no. 4, column 5 or 8, or homologs or fragments thereof, e.g., if for the nucleic acid molecule in the respective line in Table I, column 6 the term “mitochondrial” is indicated. (For the avoidance of doubt, the term “mitochondric” and “mitochondrial” are interchangeable.)

[0074.1.1.4] to [0075.1.1.4] for the disclosure of these paragraphs see [0074.1.1.1] to [0075.1.1.1] above.

Nucleic acid sequences coding for the transit peptides may be chemically synthesized either in part or wholly according to structure of transit peptide sequences disclosed in the prior art. Said natural or chemically synthesized sequences can be directly linked to the sequences encoding the mature protein or via a linker nucleic acid sequence, which may be typically less than 500 base pairs, preferably less than 450, 400, 350, 300, 250 or 200 base pairs, more preferably less than 150, 100, 90, 80, 70, 60, 50, 40 or 30 base pairs and most preferably less than 25, 20, 15, 12, 9, 6 or 3 base pairs in length and are in frame to the coding sequence. Furthermore favorable nucleic acid sequences encoding transit peptides may comprise sequences derived from more than one biological and/or chemical source and may include a nucleic acid sequence derived from the amino-terminal region of the mature protein, which in its native state is linked to the transit peptide. In a preferred embodiment of the invention said amino-terminal region of the mature protein is typically less than 150 amino acids, preferably less than 140, 130, 120, 110, 100 or 90 amino acids, more preferably less than 80, 70, 60, 50, 40, 35, 30, 25 or 20 amino acids and most preferably less than 19, 18, 17, 16, 15, 14, 13, 12, 11 or 10 amino acids in length. But even shorter or longer stretches are also possible. In addition target sequences, which facilitate the transport of proteins to other cell compartments such as the vacuole, endoplasmic reticulum, golgi complex, glyoxysomes, peroxisomes, mitochondria (in case of a plastidal transit peptide) or plastids, especially chloroplasts (in case of a mitochondric transit peptide) may be also part of the inventive nucleic acid sequence. The proteins translated from said inventive nucleic acid sequences are a kind of fusion proteins that means the nucleic acid sequences encoding the plastidal transit peptide for example the ones shown in Table a, preferably the last one of the Table a are joint to the respective nucleic acid sequences shown in Table I, application no. 4, columns 5 or 8, especially the coding region thereof, or homologs or fragments thereof, in case of nucleic acid sequences being expressed in plastids, and that means the nucleic acid sequences encoding the mitochondric transit peptide for example the ones shown in Table b, preferably the last one of the Table b are joint to the respective nucleic acid sequences shown in Table I, application no. 4, columns 5 or 8, especially the coding region thereof, or homologs or fragments thereof, in case of nucleic acid sequences being expressed in mitochondria. The person skilled in the art is able to join said sequences in a functional manner. Advantageously the transit peptide part is cleaved off from the protein part shown in Table II, application no. 4, columns 5 or 8, or a homolog or fragment thereof, during or after the transport into the organelles, preferably into the plastids or mitochondria, respectively.

All products of the cleavage of the preferred transit peptide shown in the last line of Table a have preferably the N-terminal amino acid sequences QIA CSS or QIA EFQLTT in front of the start methionine of the respective protein mentioned in Table II, application no. 4, column 5 or 8. or homologs or fragments thereof. Other short amino acid sequences of an range of 1 to 20 amino acids preferable 2 to 15 amino acids, more preferable 3 to 10 amino acids most preferably 4 to 8 amino acids are also possible in front of the start methionine of the protein mentioned in Table II, application no. 4, columns 5 or 8, or homologs or fragments thereof. In case of the amino acid sequence QIA CSS the three amino acids in front of the start methionine are stemming from the LIC (=ligatation independent cloning) cassette. Said short amino acid sequence is preferred in the case of the expression of E. coli genes. In case of the amino acid sequence QIA EFQLTT the six amino acids in front of the start methionine are stemming from the LIC cassette. Said short amino acid sequence is preferred in the case of the expression of S. cerevisiae genes. The skilled worker knowns that other short sequences are also useful in the expression of the respective nucleic acid molecule comprising a polynucleotide as depicted in Table I, application no. 4, column 5 or 8, preferably the coding region thereof, or a homolog or a fragment thereof. Furthermore the skilled worker is aware of the fact that there is not a need for such short sequences in the expression of the genes.

[0077.1.1.4] to [0078.1.1.4] for the disclosure of these paragraphs see [0077.1.1.1] to [0078.1.1.1] above.

Alternatively to the targeting of the respective sequences shown in Table II, application no. 4, columns 5 or 8, or homologs or fragments thereof, preferably of sequences in general encoded in the nucleus with the aid of the targeting sequences mentioned for example in Table a alone, Table b alone, or in combination with other targeting sequences preferably into plastids or mitochondria, respectively, the nucleic acids of the invention can directly be introduced into the plastidal or mitochondrial genome. Therefore in a preferred embodiment the respective nucleic acid sequences shown in the respective line in Table I, application no. 4, columns 5 or 8, preferably the coding region thereof, or homologs or fragments thereof, are directly introduced and expressed in plastids e.g., if for the nucleic acid molecule in the respective line in Table I, column 6, the term “plastidic” is indicated, or are directly introduced and expressed in mitochondria e.g., if for the nucleic acid molecule in the respective line in Table I, column 6 the term “mitochondrial” is indicated.

The term “introduced” in the context of this specification shall mean the insertion of a nucleic acid sequence into the organism by means of a “transfection”, “transduction” or preferably by “transformation”.

A plastid, such as a chloroplast, or a mitochondrion, has been “transformed” by an exogenous (preferably foreign) nucleic acid sequence if a nucleic acid sequence has been introduced into the plastid or mitochondrion that means that this sequence has crossed the membrane or the membranes of the plastid or of the mitochondrion. The exogenous, preferably foreign, DNA may be integrated (covalently linked) into plastid or mitochondrial DNA making up the genome of the plastid or mitochondrion, or it may remain unintegrated (e.g., by including a chloroplast origin of replication). “Stably” integrated DNA sequences are those, which are inherited through plastid or mitochondrion replication, thereby transferring new plastids or mitochondria, with the features of the integrated DNA sequence to the progeny.

[0080.1.1.4] to [0083.1.1.4] for the disclosure of this paragraph see [0080.1.1.1] to [0083.1.1.1] above.

For the process of the present invention it may be of great advantage that by transforming the plastids the intraspecies specific transgene flow is blocked, because a lot of species such as corn, cotton and rice have a strict maternal inheritance of plastids. By placing the respective genes specified in the respective line in Table I, application no. 4, columns 5 or 8, preferably the coding region, or homologs or fragments thereof, e.g. if for the nucleic acid molecule in the respective line in column 6 of Table I the term “plastidic” is indicated, in the plastids of plants, these genes will not be present in the pollen of said plants.

A further preferred embodiment of the invention relates to the use of so called “chloroplast localization sequences”, in which a first RNA sequence or molecule is capable of transporting or “chaperoning” a second RNA sequence, such as a RNA sequence transcribed from the sequences depicted in the respective line in Table I, application no. 4, columns 5 or 8, preferably the coding region thereof, or a homolog or a fragment thereof, or a sequence encoding a protein, as depicted in the respective line in Table II, application no. 4, columns 5 and 8, or a homolog or a fragment thereof, from an external environment inside a cell or outside a plastid into a chloroplast. In one embodiment the chloroplast localization signal is substantially similar or complementary to a complete or intact viroid sequence. The chloroplast localization signal may be encoded by a DNA sequence, which is transcribed into the chloroplast localization RNA. The term “viroid” refers to a naturally occurring single stranded RNA molecule (Flores, C. R. Acad. Sci. III. 324 (10), 943 (2001)). Viroids usually contain about 200-500 nucleotides and generally exist as circular molecules. Examples of viroids that contain chloroplast localization signals include but are not limited to ASBVd, PLMVd, CChMVd and ELVd. The viroid sequence or a functional part of it can be fused to the sequences depicted in the respective line in Table I, application no. 4, columns 5 or 8, preferably the coding region thereof, or a homolog or a fragment thereof, or a sequence encoding a protein, as depicted in the respective line in Table II, application no. 4, columns 5 or 8, or a homolog or a fragment thereof, in such a manner that the viroid sequence transports a sequence transcribed from a sequence as depicted in the respective line in Table I, application no. 4 columns 5 or 8, preferably the coding region thereof, or a homolog or a fragment thereof, or a sequence encoding a protein as depicted in the respective line in Table II, application no. 4 columns 5 or 8, or a homolog or a fragment thereof, into the chloroplasts, e.g. if for the nucleic acid molecule in column 6 of Table I the term “plastidic” is indicated. A preferred embodiment uses a modified ASBVd (Navarro et al., Virology. 268 (1), 218 (2000)).

In a further specific embodiment the protein to be expressed in the plastids such as the proteins depicted in the respective line in Table II, application no. 4, columns 5 or 8, or a homolog or a fragment thereof, e.g. if for the nucleic acid molecule in the respective line in column 6 of Table I the term “plastidic” is indicated, are encoded by different nucleic acids. Such a method is disclosed in WO 2004/040973, which shall be incorporated by reference. WO 2004/040973 teaches a method, which relates to the translocation of an RNA corresponding to a gene or gene fragment into the chloroplast by means of a chloroplast localization sequence. The genes, which should be expressed in the plant or plant cells, are split into nucleic acid fragments, which are introduced into different compartments in the plant e.g. the nucleus, the plastids and/or mitochondria. Additionally plant cells are described in which the chloroplast contains a ribozyme fused at one end to an RNA encoding a fragment of a protein used in the inventive process such that the ribozyme can trans-splice the translocated fusion RNA to the RNA encoding the gene fragment to form and as the case may be reunite the nucleic acid fragments to an intact mRNA encoding a functional protein for example as disclosed in the respective line in Table II, application no. 4, columns 5 or 8, or a homolog or a fragment thereof.

In a preferred embodiment of the invention the nucleic acid sequences as shown in the respective line in Table I, application no. 4, columns 5 or 8, preferably the coding region thereof, or homologs or fragments thereof, e.g. if for the nucleic acid molecule in the respective line in column 6 of Table I the term “plastidic” is indicated, used in the inventive process are transformed into plastids, which are metabolically active. Those plastids should preferably maintain at a high copy number in the plant or plant tissue of interest, most preferably the chloroplasts found in green plant tissues, such as leaves or cotyledons or in seeds.

For a good expression in the plastids the nucleic acid sequences as shown in the respective line in Table I, application no. 4, columns 5 or 8, preferably the coding region thereof, or homologs or fragments thereof, e.g. if for the nucleic acid molecule in the respective line in column 6 of Table I the term “plastidic” is indicated, are introduced into an expression cassette using preferably a promoter and terminater, which are active in plastids preferably a chloroplast promoter. Examples of such promoters include the psbA promoter from the gene from spinach or pea, the rbcL promoter, and the atpB promoter from corn.

[0089.1.1.4] to [0092.1.1.4] for the disclosure of these paragraphs see [0089.1.1.1] to [0092.1.1.1] above.

Advantageously the process for the production of the fine chemical threonine leads to an enhanced production of the respective fine chemical. The terms “enhanced” or “increase” mean at least a 10%, 20%, 30%, 40% or 50%, preferably at least 60%, 70%, 80%, 90% or 100%, more preferably 150%, 200%, 300%, 400% or 500% higher production of the respective fine chemical threonine in comparison to the wild-type as defined above, e.g. that means in comparison to a non-human organism without the aforementioned modification of the activity of a protein as shown in the respective line in Table II, application no. 4, column 5 or 8, or a fragment or a homolog thereof. The modification of the activity of a protein as shown in the respective line in Table II, application no. 4, column 5 or 8, or a homolog or a fragment thereof, or their combination can be achieved by joining the protein to a respective transit peptide, e.g. if for the respective encoding nucleic acid molecule in column 6 of Table I the term “plastidic” or “mitochondrial” is indicated.

Surprisingly it was found, that the transgenic expression of a protein as shown in the respective line in Table II, application no. 4, column 5, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, conferred a production of or an increase in threonine,respectively, to the transgenic non-human organism as compared to a corresponding non-transformed wild type.

In one embodiment the transgenic expression of a protein as shown in the respective line in Table II, application no. 4, column 5 or 8, or a homolog or a fragment thereof, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, conferred a production of or an increase in threonine,respectively, to the transgenic non-human organism as compared to a corresponding non-transformed wild type.

Accordingly, in one embodiment, an increase of threonine in a non-human organism, as compared to a corresponding non-transformed wild type non-human organism, is conferred in the process of the invention, if the activity of a polypeptide showing the activity of a 47266012-protein, or if the activity of the polypeptide 47266012_SOYBEAN, preferably represented by SEQ ID NO. 15188, or a homolog or fragment thereof, or if the activity of a polypeptide encoded by a nucleic acid molecule comprising the nucleic acid SEQ ID NO. 15187, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 15187 or polypeptide SEQ ID NO. 15188, respectively, or a homolog or a fragment thereof, is increased or generated, or if the activity 47266012-protein is increased or generated in a non-human organism, like a microorganism or a plant cell, plant or part thereof, especially with non-targeted localization, For example, an increase of the threonine of at least 1 percent, particularly in a range of 19 to 26-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a 49747384_SOYBEAN-protein, or if the activity of the polypeptide 49747384_SOYBEAN, preferably represented by SEQ ID NO. 70, or a homolog or fragment thereof, or if the activity of a polypeptide encoded by a nucleic acid molecule comprising the nucleic acid SEQ ID NO. 69, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 69 or polypeptide SEQ ID NO. 70, respectively, or a homolog or a fragment thereof, is increased or generated, or if the activity 49747384_SOYBEAN-protein is increased or generated in a non-human organism, like a microorganism or a plant cell, plant or part thereof, especially with non-targeted localization, For example, an increase of the threonine of at least 1 percent, particularly in a range of 31 to 100 -percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a glucose-6-phosphate 1-dehydrogenase, or if the activity of the polypeptide 59582753_SOYBEAN, preferably represented by SEQ ID NO. 16884, or a homolog or fragment thereof, or if the activity of a polypeptide encoded by a nucleic acid molecule comprising the nucleic acid SEQ ID NO. 16883, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 16883 or polypeptide SEQ ID NO. 16884, respectively, or a homolog or a fragment thereof, is increased or generated, or if the activity glucose-6-phosphate 1-dehydrogenase is increased or generated in a non-human organism, like a microorganism or a plant cell, plant or part thereof, especially with plastidic localization, For example, an increase of the threonine of at least 1 percent, partitularly in a range of 16 to 54 -percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a At1g09680-protein, or if the activity of the polypeptide At1g09680, preferably represented by SEQ ID NO. 353, or a homolog or fragment thereof, or if the activity of a polypeptide encoded by a nucleic acid molecule comprising the nucleic acid SEQ ID NO. 352, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 352 or polypeptide SEQ ID NO. 353, respectively, or a homolog or a fragment thereof, is increased or generated, or if the activity At1g09680-protein is increased or generated in a non-human organism, like a microorganism or a plant cell, plant or part thereof, especially with non-targeted localization, For example, an increase of the threonine of at least 1 percent, particularly in a range of 18 to 69-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a bifunctional aspartokinase/homoserine dehydrogenase, or if the activity of the polypeptide At1g31230, preferably represented by SEQ ID NO. 628, or a homolog or fragment thereof, or if the activity of a polypeptide encoded by a nucleic acid molecule comprising the nucleic acid SEQ ID NO. 627, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 627 or polypeptide SEQ ID NO. 628, respectively, or a homolog or a fragment thereof, is increased or generated, or if the activity bifunctional aspartokinase/homoserine dehydrogenase is increased or generated in a non-human organism, like a microorganism or a plant cell, plant or part thereof, especially with non-targeted localization, For example, an increase of the threonine of at least 1 percent, particularly in a range of 231 to 2283-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a eukaryotic translation initiation factor 5, or if the activity of the polypeptide At1g36730, preferably represented by SEQ ID NO. 17969, or a homolog or fragment thereof, or if the activity of a polypeptide encoded by a nucleic acid molecule comprising the nucleic acid SEQ ID NO. 17968, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 17968 or polypeptide SEQ ID NO. 17969, respectively, or a homolog or a fragment thereof, is increased or generated, or if the activity eukaryotic translation initiation factor 5 is increased or generated in a non-human organism, like a microorganism or a plant cell, plant or part thereof, especially with non-targeted localization, For example, an increase of the threonine of at least 1 percent, particularly in a range of 18 to 84-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a transcriptional regulator, or if the activity of the polypeptide At1g43850, preferably represented by SEQ ID NO. 18071, or a homolog or fragment thereof, or if the activity of a polypeptide encoded by a nucleic acid molecule comprising the nucleic acid SEQ ID NO. 18070, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 18070 or polypeptide SEQ ID NO. 18071, respectively, or a homolog or a fragment thereof, is increased or generated, or if the activity transcriptional regulator is increased or generated in a non-human organism, like a microorganism or a plant cell, plant or part thereof, especially with non-targeted localization, For example, an increase of the threonine of at least 1 percent, particularly in a range of 18 to 144-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a protein phosphatase, or if the activity of the polypeptide At1g48040, preferably represented by SEQ ID NO. 813, or a homolog or fragment thereof, or if the activity of a polypeptide encoded by a nucleic acid molecule comprising the nucleic acid SEQ ID NO. 812, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 812 or polypeptide SEQ ID NO. 813, respectively, or a homolog or a fragment thereof, is increased or generated, or if the activity protein phosphatase is increased or generated in a non-human organism, like a microorganism or a plant cell, plant or part thereof, especially with non-targeted localization, For example, an increase of the threonine of at least 1 percent, particularly in a range of 24 to 88-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a protein kinase, or if the activity of the polypeptide At1g48260, preferably represented by SEQ ID NO. 18236, or a homolog or fragment thereof, or if the activity of a polypeptide encoded by a nucleic acid molecule comprising the nucleic acid SEQ ID NO. 18235, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 18235 or polypeptide SEQ ID NO. 18236, respectively, or a homolog or a fragment thereof, is increased or generated, or if the activity protein kinase is increased or generated in a non-human organism, like a microorganism or a plant cell, plant or part thereof, especially with non-targeted localization, For example, an increase of the threonine of at least 1 percent, particularly in a range of 29 to 127-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a protein phosphatase, or if the activity of the polypeptide At1g72770, preferably represented by SEQ ID NO. 19420, or a homolog or fragment thereof, or if the activity of a polypeptide encoded by a nucleic acid molecule comprising the nucleic acid SEQ ID NO. 19419, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 19419 or polypeptide SEQ ID NO. 19420, respectively, or a homolog or a fragment thereof, is increased or generated, or if the activity protein phosphatase is increased or generated in a non-human organism, like a microorganism or a plant cell, plant or part thereof, especially with non-targeted localization, For example, an increase of the threonine of at least 1 percent, particularly in a range of 41 to 302-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a protein phosphatase, or if the activity of the polypeptide At2g25070, preferably represented by SEQ ID NO. 1299, or a homolog or fragment thereof, or if the activity of a polypeptide encoded by a nucleic acid molecule comprising the nucleic acid SEQ ID NO. 1298, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 1298 or polypeptide SEQ ID NO. 1299, respectively, or a homolog or a fragment thereof, is increased or generated, or if the activity protein phosphatase is increased or generated in a non-human organism, like a microorganism or a plant cell, plant or part thereof, especially with non-targeted localization, For example, an increase of the threonine of at least 1 percent, particularly in a range of 27 to 148-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a glutaredoxin, or if the activity of the polypeptide At2g30540, preferably represented by SEQ ID NO. 20347, or a homolog or fragment thereof, or if the activity of a polypeptide encoded by a nucleic acid molecule comprising the nucleic acid SEQ ID NO. 20346, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 20346 or polypeptide SEQ ID NO. 20347, respectively, or a homolog or a fragment thereof, is increased or generated, or if the activity glutaredoxin is increased or generated in a non-human organism, like a microorganism or a plant cell, plant or part thereof, especially with non-targeted localization, For example, an increase of the threonine of at least 1 percent, particularly in a range of 21 to 74-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a CCAAT-binding transcription factor, or if the activity of the polypeptide At3g20910, preferably represented by SEQ ID NO. 22922, or a homolog or fragment thereof, or if the activity of a polypeptide encoded by a nucleic acid molecule comprising the nucleic acid SEQ ID NO. 22921, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 22921 or polypeptide SEQ ID NO. 22922, respectively, or a homolog or a fragment thereof, is increased or generated, or if the activity CCAAT-binding transcription factor is increased or generated in a non-human organism, like a microorganism or a plant cell, plant or part thereof, especially with non-targeted localization, For example, an increase of the threonine of at least 1 percent, particularly in a range of 19 to 52-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a protein kinase, or if the activity of the polypeptide At3g23000, preferably represented by SEQ ID NO. 1816, or a homolog or fragment thereof, or if the activity of a polypeptide encoded by a nucleic acid molecule comprising the nucleic acid SEQ ID NO. 1815, preferably the coding region thereof, or a homolog or fragment thereof, e.g. derived from Arabidopsis thaliana, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 1815 or polypeptide SEQ ID NO. 1816, respectively, or a homolog or a fragment thereof, is increased or generated, or if the activity protein kinase is increased or generated in a non-human organism, like a microorganism or a plant cell, plant or part thereof, especially with non-targeted localization, For example, an increase of the threonine of at least 1 percent, particularly in a range of 16 to 83-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a glycosyl transferase, or if the activity of the polypeptide At3g27540, preferably represented by SEQ ID NO. 68778, or a homolog or fragment thereof, or if the activity of a polypeptide encoded by a nucleic acid molecule comprising the nucleic acid SEQ ID NO. 68777, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 68777 or polypeptide SEQ ID NO. 68778, respectively, or a homolog or a fragment thereof, is increased or generated, or if the activity glycosyl transferase is increased or generated in a non-human organism, like a microorganism or a plant cell, plant or part thereof, especially with non-targeted localization, For example, an increase of the threonine of at least 1 percent, particularly in a range of 20 to 48-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a monthiol glutaredoxin, or if the activity of the polypeptide At3g62930, preferably represented by SEQ ID NO. 2574, or a homolog or fragment thereof, or if the activity of a polypeptide encoded by a nucleic acid molecule comprising the nucleic acid SEQ ID NO. 2573, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 2573 or polypeptide SEQ ID NO. 2574, respectively, or a homolog or a fragment thereof, is increased or generated, or if the activity monthiol glutaredoxin is increased or generated in a non-human organism, like a microorganism or a plant cell, plant or part thereof, especially with non-targeted localization, For example, an increase of the threonine of at least 1 percent, particularly in a range of 28 to 49-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, For example, an increase of the threonine of at least 1 percent, particularly in a range of 32 to 130-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization, For example, an increase of the threonine of at least 1 percent, particularly in a range of 31 to 672-percent is conferred as compared to a corresponding non-transformed wild type non-human organism

Accordingly, in one embodiment, an increase of threonine in a non-human organism, as compared to a corresponding non-transformed wild type non-human organism, is conferred in the process of the invention, if the activity of a polypeptide showing the activity of a monothiol glutaredoxin, or if the activity of the polypeptide At4g15670, preferably represented by SEQ ID NO. 3280, or a homolog or fragment thereof, or if the activity of a polypeptide encoded by a nucleic acid molecule comprising the nucleic acid SEQ ID NO. 3279, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 3279 or polypeptide SEQ ID NO. 3280, respectively, or a homolog or a fragment thereof, is increased or generated, or if the activity monothiol glutaredoxin is increased or generated in a non-human organism, like a microorganism or a plant cell, plant or part thereof, especially with non-targeted localization, For example, an increase of the threonine of at least 1 percent, particularly in a range of 29 to 92-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a monothiol glutaredoxin, or if the activity of the polypeptide At4g15690, preferably represented by SEQ ID NO. 23845, or a homolog or fragment thereof, or if the activity of a polypeptide encoded by a nucleic acid molecule comprising the nucleic acid SEQ ID NO. 23844, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 23844 or polypeptide SEQ ID NO. 23845, respectively, or a homolog or a fragment thereof, is increased or generated, or if the activity monothiol glutaredoxin is increased or generated in a non-human organism, like a microorganism or a plant cell, plant or part thereof, especially with non-targeted localization, For example, an increase of the threonine of at least 1 percent, particularly in a range of 26 to 59-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a heat shock transcription factor, or if the activity of the polypeptide At4g18880, preferably represented by SEQ ID NO. 24233, or a homolog or fragment thereof, or if the activity of a polypeptide encoded by a nucleic acid molecule comprising the nucleic acid SEQ ID NO. 24232, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 24232 or polypeptide SEQ ID NO. 24233, respectively, or a homolog or a fragment thereof, is increased or generated, or if the activity heat shock transcription factor is increased or generated in a non-human organism, like a microorganism or a plant cell, plant or part thereof, especially with non-targeted localization, For example, an increase of the threonine of at least 1 percent, particularly in a range of 32 to 75-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a At4g32480-protein, or if the activity of the polypeptide At4g32480, preferably represented by SEQ ID NO. 4041, or a homolog or fragment thereof, or if the activity of a polypeptide encoded by a nucleic acid molecule comprising the nucleic acid SEQ ID NO. 4040, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 4040 or polypeptide SEQ ID NO. 4041, respectively, or a homolog or a fragment thereof, is increased or generated, or if the activity At4g32480-protein is increased or generated in a non-human organism, like a microorganism or a plant cell, plant or part thereof, especially with non-targeted localization, For example, an increase of the threonine of at least 1 percent, particularly in a range of 32 to 265-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a cyclin D , or if the activity of the polypeptide At4g34160, preferably represented by SEQ ID NO. 24312, or a homolog or fragment thereof, or if the activity of a polypeptide encoded by a nucleic acid molecule comprising the nucleic acid SEQ ID NO. 24311, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 24311 or polypeptide SEQ ID NO. 24312, respectively, or a homolog or a fragment thereof, is increased or generated, or if the activity cyclin D is increased or generated in a non-human organism, like a microorganism or a plant cell, plant or part thereof, especially with non-targeted localization, For example, an increase of the threonine of at least 1 percent, particularly in a range of 40 to 97-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization, For example, an increase of the threonine of at least 1 percent, particularly in a range of 29 to 102-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a gibberellin 20-oxidase, or if the activity of the polypeptide At5g07200, preferably represented by SEQ ID NO. 24493, or a homolog or fragment thereof, or if the activity of a polypeptide encoded by a nucleic acid molecule comprising the nucleic acid SEQ ID NO. 24492, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 24492 or polypeptide SEQ ID NO. 24493, respectively, or a homolog or a fragment thereof, is increased or generated, or if the activity gibberellin 20-oxidase is increased or generated in a non-human organism, like a microorganism or a plant cell, plant or part thereof, especially with non-targeted localization, For example, an increase of the threonine of at least 1 percent, particularly in a range of 18 to 54-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a aspartate kinase, or if the activity of the polypeptide At5g13280, preferably represented by SEQ ID NO. 93452, or a homolog or fragment thereof, or if the activity of a polypeptide encoded by a nucleic acid molecule comprising the nucleic acid SEQ ID NO. 93451, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 93451 or polypeptide SEQ ID NO. 93452, respectively, or a homolog or a fragment thereof, is increased or generated, or if the activity aspartate kinase is increased or generated in a non-human organism, like a microorganism or a plant cell, plant or part thereof, especially with non-targeted localization, For example, an increase of the threonine of at least 1 percent, particularly in a range of 38 to 356-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a At5g16650-protein, or if the activity of the polypeptide At5g16650, preferably represented by SEQ ID NO. 25284, or a homolog or fragment thereof, or if the activity of a polypeptide encoded by a nucleic acid molecule comprising the nucleic acid SEQ ID NO. 25283, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 25283 or polypeptide SEQ ID NO. 25284, respectively, or a homolog or a fragment thereof, is increased or generated, or if the activity At5g16650-protein is increased or generated in a non-human organism, like a microorganism or a plant cell, plant or part thereof, especially with non-targeted localization, For example, an increase of the threonine of at least 1 percent, particularly in a range of 16 to 48-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a thioredoxin, or if the activity of the polypeptide At5g39950, preferably represented by SEQ ID NO. 69575, or a homolog or fragment thereof, or if the activity of a polypeptide encoded by a nucleic acid molecule comprising the nucleic acid SEQ ID NO. 69574, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 69574 or polypeptide SEQ ID NO. 69575, respectively, or a homolog or a fragment thereof, is increased or generated, or if the activity thioredoxin is increased or generated in a non-human organism, like a microorganism or a plant cell, plant or part thereof, especially with non-targeted localization, For example, an increase of the threonine of at least 1 percent, particularly in a range of 16 to 23-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a protein phosphatase, or if the activity of the polypeptide At5g57050, preferably represented by SEQ ID NO. 5319, or a homolog or fragment thereof, or if the activity of a polypeptide encoded by a nucleic acid molecule comprising the nucleic acid SEQ ID NO. 5318, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 5318 or polypeptide SEQ ID NO. 5319, respectively, or a homolog or a fragment thereof, is increased or generated, or if the activity protein phosphatase is increased or generated in a non-human organism, like a microorganism or a plant cell, plant or part thereof, especially with non-targeted localization, For example, an increase of the threonine of at least 1 percent, particularly in a range of 18 to 60-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a zinc finger protein, or if the activity of the polypeptide At5g64920, preferably represented by SEQ ID NO. 5494, or a homolog or fragment thereof, or if the activity of a polypeptide encoded by a nucleic acid molecule comprising the nucleic acid SEQ ID NO. 5493, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 5493 or polypeptide SEQ ID NO. 5494, respectively, or a homolog or a fragment thereof, is increased or generated, or if the activity zinc finger protein is increased or generated in a non-human organism, like a microorganism or a plant cell, plant or part thereof, especially with non-targeted localization, For example, an increase of the threonine of at least 1 percent, particularly in a range of 30 to 578 percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a CDP-diacylglycerol-serine O-phosphatidyltransferase, or if the activity of the polypeptide AvinDRAFT_{—}0035, preferably represented by SEQ ID NO. 70584, or a homolog or fragment thereof, or if the activity of a polypeptide encoded by a nucleic acid molecule comprising the nucleic acid SEQ ID NO. 70583, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 70583 or polypeptide SEQ ID NO. 70584, respectively, or a homolog or a fragment thereof, is increased or generated, or if the activity CDP-diacylglycerol-serine O-phosphatidyltransferase is increased or generated in a non-human organism, like a microorganism or a plant cell, plant or part thereof, especially with non-targeted localization, For example, an increase of the threonine of at least 1 percent, particularly in a range of 17 to 41-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a malic enzyme, or if the activity of the polypeptide AvinDRAFT_{—}1495, preferably represented by SEQ ID NO. 5558, or a homolog or fragment thereof, or if the activity of a polypeptide encoded by a nucleic acid molecule comprising the nucleic acid SEQ ID NO. 5557, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 5557 or polypeptide SEQ ID NO. 5558, respectively, or a homolog or a fragment thereof, is increased or generated, or if the activity malic enzyme is increased or generated in a non-human organism, like a microorganism or a plant cell, plant or part thereof, especially with non-targeted localization, For example, an increase of the threonine of at least 1 percent, particularly in a range of 33 to 157-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a secindependent protein translocase, or if the activity of the polypeptide AvinDRAFT_{—}1624, preferably represented by SEQ ID NO. 26197, or a homolog or fragment thereof, or if the activity of a polypeptide encoded by a nucleic acid molecule comprising the nucleic acid SEQ ID NO. 26196, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 26196 or polypeptide SEQ ID NO. 26197, respectively, or a homolog or a fragment thereof, is increased or generated, or if the activity sec-independent protein translocase is increased or generated in a non-human organism, like a microorganism or a plant cell, plant or part thereof, especially with non-targeted localization, For example, an increase of the threonine of at least 1 percent, particularly in a range of 18 to 55-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a beta-hydroxylase, or if the activity of the polypeptide AvinDRAFT_{—}2091, preferably represented by SEQ ID NO. 6041, or a homolog or fragment thereof, or if the activity of a polypeptide encoded by a nucleic acid molecule comprising the nucleic acid SEQ ID NO. 6040, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 6040 or polypeptide SEQ ID NO. 6041, respectively, or a homolog or a fragment thereof, is increased or generated, or if the activity beta-hydroxylase is increased or generated in a non-human organism, like a microorganism or a plant cell, plant or part thereof, especially with non-targeted localization, For example, an increase of the threonine of at least 1 percent, particularly in a range of 19 to 37-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a elongation facfor Tu, or if the activity of the polypeptide AvinDRAFT_{—}2344, preferably represented by SEQ ID NO. 27022, or a homolog or fragment thereof, or if the activity of a polypeptide encoded by a nucleic acid molecule comprising the nucleic acid SEQ ID NO. 27021, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 27021 or polypeptide SEQ ID NO. 27022, respectively, or a homolog or a fragment thereof, is increased or generated, or if the activity elongation factor Tu is increased or generated in a non-human organism, like a microorganism or a plant cell, plant or part thereof, especially with non-targeted localization, For example, an increase of the threonine of at least 1 percent, particularly in a range of 25 to 121-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a ABC transporter permease protein, or if the activity of the polypeptide AvinDRAFT_{—}2521, preferably represented by SEQ ID NO. 27883, or a homolog or fragment thereof, or if the activity of a polypeptide encoded by a nucleic acid molecule comprising the nucleic acid SEQ ID NO. 27882, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 27882 or polypeptide SEQ ID NO. 27883, respectively, or a homolog or a fragment thereof, is increased or generated, or if the activity ABC transporter permease protein is increased or generated in a non-human organism, like a microorganism or a plant cell, plant or part thereof, especially with non-targeted localization, For example, an increase of the threonine of at least 1 percent, particularly in a range of 33 to 116-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a acyl-CoA synthase, or if the activity of the polypeptide AvinDRAFT_{—}2754, preferably represented by SEQ ID NO. 28041, or a homolog or fragment thereof, or if the activity of a polypeptide encoded by a nucleic acid molecule comprising the nucleic acid SEQ ID NO. 28040, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 28040 or polypeptide SEQ ID NO. 28041, respectively, or a homolog or a fragment thereof, is increased or generated, or if the activity acyl-CoA synthase is increased or generated in a non-human organism, like a microorganism or a plant cell, plant or part thereof, especially with non-targeted localization, For example, an increase of the threonine of at least 1 percent, particularly in a range of 17 to 61-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a 2-oxoglutarate dehydrogenase E1 subunit, or if the activity of the polypeptide AvinDRAFT_{—}3028, preferably represented by SEQ ID NO. 6076, or a homolog or fragment thereof, or if the activity of a polypeptide encoded by a nucleic acid molecule comprising the nucleic acid SEQ ID NO. 6075, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 6075 or polypeptide SEQ ID NO. 6076, respectively, or a homolog or a fragment thereof, is increased or generated, or if the activity 2-oxoglutarate dehydrogenase E1 subunit is increased or generated in a non-human organism, like a microorganism or a plant cell, plant or part thereof, especially with non-targeted localization, For example, an increase of the threonine of at least 1 percent, particularly in a range of 29 to 249-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a permease protein of peptide ABC transporter, or if the activity of the polypeptide AvinDRAFT_{—}3086, preferably represented by SEQ ID NO. 93533, or a homolog or fragment thereof, or if the activity of a polypeptide encoded by a nucleic acid molecule comprising the nucleic acid SEQ ID NO. 93532, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 93532 or polypeptide SEQ ID NO. 93533, respectively, or a homolog or a fragment thereof, is increased or generated, or if the activity permease protein of peptide ABC transporter is increased or generated in a non-human organism, like a microorganism or a plant cell, plant or part thereof, especially with non-targeted localization, For example, an increase of the threonine of at least 1 percent, particularly in a range of 24 to 34-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a GDP-mannose dehydrogenase, or if the activity of the polypeptide AvinDRAFT_{—}3135, preferably represented by SEQ ID NO. 72347, or a homolog or fragment thereof, or if the activity of a polypeptide encoded by a nucleic acid molecule comprising the nucleic acid SEQ ID NO. 72346, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 72346 or polypeptide SEQ ID NO. 72347, respectively, or a homolog or a fragment thereof, is increased or generated, or if the activity GDP-mannose dehydrogenase is increased or generated in a non-human organism, like a microorganism or a plant cell, plant or part thereof, especially with non-targeted localization, For example, an increase of the threonine of at least 1 percent, partitularly in a range of 21 to 25-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a enoyl-CoA hydratase, or if the activity of the polypeptide AvinDRAFT_{—}3159, preferably represented by SEQ ID NO. 28739, or a homolog or fragment thereof, or if the activity of a polypeptide encoded by a nucleic acid molecule comprising the nucleic acid SEQ ID NO. 28738, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 28738 or polypeptide SEQ ID NO. 28739, respectively, or a homolog or a fragment thereof, is increased or generated, or if the activity enoyl-CoA hydratase is increased or generated in a non-human organism, like a microorganism or a plant cell, plant or part thereof, especially with non-targeted localization, For example, an increase of the threonine of at least 1 percent, particularly in a range of 20 to 139-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a transcriptional regulator, or if the activity of the polypeptide AvinDRAFT_{—}3209, preferably represented by SEQ ID NO. 29287, or a homolog or fragment thereof, or if the activity of a polypeptide encoded by a nucleic acid molecule comprising the nucleic acid SEQ ID NO. 29286, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 29286 or polypeptide SEQ ID NO. 29287, respectively, or a homolog or a fragment thereof, is increased or generated, or if the activity transcriptional regulator is increased or generated in a non-human organism, like a microorganism or a plant cell, plant or part thereof, especially with non-targeted localization, For example, an increase of the threonine of at least 1 percent, particularly in a range of 18 to 42-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a homoserine dehydrogenase, or if the activity of the polypeptide AvinDRAFT_{—}3546, preferably represented by SEQ ID NO. 6269, or a homolog or fragment thereof, or if the activity of a polypeptide encoded by a nucleic acid molecule comprising the nucleic acid SEQ ID NO. 6268, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 6268 or polypeptide SEQ ID NO. 6269, respectively, or a homolog or a fragment thereof, is increased or generated, or if the activity homoserine dehydrogenase is increased or generated in a non-human organism, like a microorganism or a plant cell, plant or part thereof, especially with non-targeted localization, For example, an increase of the threonine of at least 1 percent, particularly in a range of 41 to 1222-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a hydrolase, or if the activity of the polypeptide AvinDRAFT_{—}5103, preferably represented by SEQ ID NO. 6511, or a homolog or fragment thereof, or if the activity of a polypeptide encoded by a nucleic acid molecule comprising the nucleic acid SEQ ID NO. 6510, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 6510 or polypeptide SEQ ID NO. 6511, respectively, or a homolog or a fragment thereof, is increased or generated, or if the activity hydrolase is increased or generated in a non-human organism, like a microorganism or a plant cell, plant or part thereof, especially with non-targeted localization, For example, an increase of the threonine of at least 1 percent, particularly in a range of 22 to 170-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a glutamate-ammonia-ligase, or if the activity of the polypeptide AvinDRAFT_{—}5246, preferably represented by SEQ ID NO. 32038, or a homolog or fragment thereof, or if the activity of a polypeptide encoded by a nucleic acid molecule comprising the nucleic acid SEQ ID NO. 32037, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 32037 or polypeptide SEQ ID NO. 32038, respectively, or a homolog or a fragment thereof, is increased or generated, or if the activity glutamate-ammonia-ligase is increased or generated in a non-human organism, like a microorganism or a plant cell, plant or part thereof, especially with non-targeted localization, For example, an increase of the threonine of at least 1 percent, particularly in a range of 24 to 136-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a peptidase, or if the activity of the polypeptide AvinDRAFT_{—}5579, preferably represented by SEQ ID NO. 74663, or a homolog or fragment thereof, or if the activity of a polypeptide encoded by a nucleic acid molecule comprising the nucleic acid SEQ ID NO. 74662, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 74662 or polypeptide SEQ ID NO. 74663, respectively, or a homolog or a fragment thereof, is increased or generated, or if the activity peptidase is increased or generated in a non-human organism, like a microorganism or a plant cell, plant or part thereof, especially with non-targeted localization, For example, an increase of the threonine of at least 1 percent, particularly in a range of 17 to 35-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a Chaperone protein CIpB, or if the activity of the polypeptide AvinDRAFT_{—}6093, preferably represented by SEQ ID NO. 33597, or a homolog or fragment thereof, or if the activity of a polypeptide encoded by a nucleic acid molecule comprising the nucleic acid SEQ ID NO. 33596, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 33596 or polypeptide SEQ ID NO. 33597, respectively, or a homolog or a fragment thereof, is increased or generated, or if the activity Chaperone protein CIpB is increased or generated in a non-human organism, like a microorganism or a plant cell, plant or part thereof, especially with non-targeted localization, For example, an increase of the threonine of at least 1 percent, particularly in a range of 18 to 56-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a transcriptional regulator protein, or if the activity of the polypeptide AvinDRAFT_{—}6864, preferably represented by SEQ ID NO. 34205, or a homolog or fragment thereof, or if the activity of a polypeptide encoded by a nucleic acid molecule comprising the nucleic acid SEQ ID NO. 34204, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 34204 or polypeptide SEQ ID NO. 34205, respectively, or a homolog or a fragment thereof, is increased or generated, or if the activity transcriptional regulator protein is increased or generated in a non-human organism, like a microorganism or a plant cell, plant or part thereof, especially with non-targeted localization, For example, an increase of the threonine of at least 1 percent, particularly in a range of 47 to 68-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a AX653549-protein, or if the activity of the polypeptide AX653549, preferably represented by SEQ ID NO. 34302, or a homolog or fragment thereof, or if the activity of a polypeptide encoded by a nucleic acid molecule comprising the nucleic acid SEQ ID NO. 34301, preferably the coding region thereof, or a homolog or fragment thereof, e.g. derived from Oryza sativa, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 34301 or polypeptide SEQ ID NO. 34302, respectively, or a homolog or a fragment thereof, is increased or generated, or if the activity AX653549-protein is increased or generated in a non-human organism, like a microorganism or a plant cell, plant or part thereof, especially with non-targeted localization, For example, an increase of the threonine of at least 1 percent, particularly in a range of 17 to 50-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a threonine synthase, or if the activity of the polypeptide B0004, preferably represented by SEQ ID NO. 34890, or a homolog or fragment thereof, or if the activity of a polypeptide encoded by a nucleic acid molecule comprising the nucleic acid SEQ ID NO. 34889, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 34889 or polypeptide SEQ ID NO. 34890, respectively, or a homolog or a fragment thereof, is increased or generated, or if the activity threonine synthase is increased or generated in a non-human organism, like a microorganism or a plant cell, plant or part thereof, especially with plastidic localization, For example, an increase of the threonine of at least 1 percent, particularly in a range of 23 to 123-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a acetolactate synthase small subunit, or if the activity of the polypeptide B0078, preferably represented by SEQ ID NO. 6819, or a homolog or fragment thereof, or if the activity of a polypeptide encoded by a nucleic acid molecule comprising the nucleic acid SEQ ID NO. 6818, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 6818 or polypeptide SEQ ID NO. 6819, respectively, or a homolog or a fragment thereof, is increased or generated, or if the activity acetolactate synthase small subunit is increased or generated in a non-human organism, like a microorganism or a plant cell, plant or part thereof, especially with non-targeted localization, For example, an increase of the threonine of at least 1 percent, particularly in a range of 33 to 83-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a dihydrolipoamide acetyltransferase, or if the activity of the polypeptide B0115, preferably represented by SEQ ID NO. 35367, or a homolog or fragment thereof, or if the activity of a polypeptide encoded by a nucleic acid molecule comprising the nucleic acid SEQ ID NO. 35366, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 35366 or polypeptide SEQ ID NO. 35367, respectively, or a homolog or a fragment thereof, is increased or generated, or if the activity dihydrolipoamide acetyltransferase is increased or generated in a non-human organism, like a microorganism or a plant cell, plant or part thereof, especially with plastidic localization, For example, an increase of the threonine of at least 1 percent, particularly in a range of 35 to 50-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a glucose dehydrogenase, or if the activity of the polypeptide B0124, preferably represented by SEQ ID NO. 35483, or a homolog or fragment thereof, or if the activity of a polypeptide encoded by a nucleic acid molecule comprising the nucleic acid SEQ ID NO. 35482, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 35482 or polypeptide SEQ ID NO. 35483, respectively, or a homolog or a fragment thereof, is increased or generated, or if the activity glucose dehydrogenase is increased or generated in a non-human organism, like a microorganism or a plant cell, plant or part thereof, especially with non-targeted localization, For example, an increase of the threonine of at least 1 percent, particularly in a range of 20 to 127-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a serine protease, or if the activity of the polypeptide B0161, preferably represented by SEQ ID NO. 7082, or a homolog or fragment thereof, or if the activity of a polypeptide encoded by a nucleic acid molecule comprising the nucleic acid SEQ ID NO. 7081, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 7081 or polypeptide SEQ ID NO. 7082, respectively, or a homolog or a fragment thereof, is increased or generated, or if the activity serine protease is increased or generated in a non-human organism, like a microorganism or a plant cell, plant or part thereof, especially with non-targeted localization, For example, an increase of the threonine of at least 1 percent, particularly in a range of 36 to 144-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a acyl-CoA dehydrogenase, or if the activity of the polypeptide B0221, preferably represented by SEQ ID NO. 35591, or a homolog or fragment thereof, or if the activity of a polypeptide encoded by a nucleic acid molecule comprising the nucleic acid SEQ ID NO. 35590, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 35590 or polypeptide SEQ ID NO. 35591, respectively, or a homolog or a fragment thereof, is increased or generated, or if the activity acyl-CoA dehydrogenase is increased or generated in a non-human organism, like a microorganism or a plant cell, plant or part thereof, especially with non-targeted localization, For example, an increase of the threonine of at least 1 percent, particularly in a range of 18 to 53-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a beta-galactosidase, or if the activity of the polypeptide B0344, preferably represented by SEQ ID NO. 35734, or a homolog or fragment thereof, or if the activity of a polypeptide encoded by a nucleic acid molecule comprising the nucleic acid SEQ ID NO. 35733, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 35733 or polypeptide SEQ ID NO. 35734, respectively, or a homolog or a fragment thereof, is increased or generated, or if the activity beta-galactosidase is increased or generated in a non-human organism, like a microorganism or a plant cell, plant or part thereof, especially with plastidic localization, For example, an increase of the threonine of at least 1 percent, particularly in a range of 36 to 41-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a 2,3-dihydroxyphenylpropionate 1,2-dioxygenase, or if the activity of the polypeptide B0348, preferably represented by SEQ ID NO. 7270, or a homolog or fragment thereof, or if the activity of a polypeptide encoded by a nucleic acid molecule comprising the nucleic acid SEQ ID NO. 7269, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 7269 or polypeptide SEQ ID NO. 7270, respectively, or a homolog or a fragment thereof, is increased or generated, or if the activity 2,3-dihydroxyphenylpropionate 1,2-dioxygenase is increased or generated in a non-human organism, like a microorganism or a plant cell, plant or part thereof, especially with plastidic localization, For example, an increase of the threonine of at least 1 percent, particularly in a range of 36 to 157-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a ATP-binding component of a transport system, or if the activity of the polypeptide B0449, preferably represented by SEQ ID NO. 7334, or a homolog or fragment thereof, or if the activity of a polypeptide encoded by a nucleic acid molecule comprising the nucleic acid SEQ ID NO. 7333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 7333 or polypeptide SEQ ID NO. 7334, respectively, or a homolog or a fragment thereof, is increased or generated, or if the activity ATP-binding component of a transport system is increased or generated in a non-human organism, like a microorganism or a plant cell, plant or part thereof, especially with non-targeted localization, For example, an increase of the threonine of at least 1 percent, particularly in a range of 33 to 123-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a membrane transport protein, or if the activity of the polypeptide B0486, preferably represented by SEQ ID NO. 7687, or a homolog or fragment thereof, or if the activity of a polypeptide encoded by a nucleic acid molecule comprising the nucleic acid SEQ ID NO. 7686, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 7686 or polypeptide SEQ ID NO. 7687, respectively, or a homolog or a fragment thereof, is increased or generated, or if the activity membrane transport protein is increased or generated in a non-human organism, like a microorganism or a plant cell, plant or part thereof, especially with non-targeted localization, For example, an increase of the threonine of at least 1 percent, particularly in a range of 29 to 45-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a isochorismate synthase, or if the activity of the polypeptide B0593, preferably represented by SEQ ID NO. 35968, or a homolog or fragment thereof, or if the activity of a polypeptide encoded by a nucleic acid molecule comprising the nucleic acid SEQ ID NO. 35967, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 35967 or polypeptide SEQ ID NO. 35968, respectively, or a homolog or a fragment thereof, is increased or generated, or if the activity isochorismate synthase is increased or generated in a non-human organism, like a microorganism or a plant cell, plant or part thereof, especially with plastidic localization, For example, an increase of the threonine of at least 1 percent, particularly in a range of 31 to 76-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a sensor histidine kinase, or if the activity of the polypeptide B0695, preferably represented by SEQ ID NO. 93679, or a homolog or fragment thereof, or if the activity of a polypeptide encoded by a nucleic acid molecule comprising the nucleic acid SEQ ID NO. 93678, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 93678 or polypeptide SEQ ID NO. 93679, respectively, or a homolog or a fragment thereof, is increased or generated, or if the activity sensor histidine kinase is increased or generated in a non-human organism, like a microorganism or a plant cell, plant or part thereof, especially with non-targeted localization, For example, an increase of the threonine of at least 1 percent, particularly in a range of 27 to 50-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a succinyl-CoA synthetase beta chain, or if the activity of the polypeptide B0728, preferably represented by SEQ ID NO. 93794, or a homolog or fragment thereof, or if the activity of a polypeptide encoded by a nucleic acid molecule comprising the nucleic acid SEQ ID NO. 93793, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 93793 or polypeptide SEQ ID NO. 93794, respectively, or a homolog or a fragment thereof, is increased or generated, or if the activity succinyl-CoA synthetase beta chain is increased or generated in a non-human organism, like a microorganism or a plant cell, plant or part thereof, especially with plastidic localization, For example, an increase of the threonine of at least 1 percent, particularly in a range of 20 to 50-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a major facilitator superfamily transporter protein, or if the activity of the polypeptide B0898, preferably represented by SEQ ID NO. 7918, or a homolog or fragment thereof, or if the activity of a polypeptide encoded by a nucleic acid molecule comprising the nucleic acid SEQ ID NO. 7917, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 7917 or polypeptide SEQ ID NO. 7918, respectively, or a homolog or a fragment thereof, is increased or generated, or if the activity major facilitator superfamily transporter protein is increased or generated in a non-human organism, like a microorganism or a plant cell, plant or part thereof, especially with non-targeted localization, For example, an increase of the threonine of at least 1 percent, particularly in a range of 19 to 355-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a phosphoanhydride phosphorylase, or if the activity of the polypeptide B0980, preferably represented by SEQ ID NO. 36810, or a homolog or fragment thereof, or if the activity of a polypeptide encoded by a nucleic acid molecule comprising the nucleic acid SEQ ID NO. 36809, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 36809 or polypeptide SEQ ID NO. 36810, respectively, or a homolog or a fragment thereof, is increased or generated, or if the activity phosphoanhydride phosphorylase is increased or generated in a non-human organism, like a microorganism or a plant cell, plant or part thereof, especially with non-targeted localization, For example, an increase of the threonine of at least 1 percent, particularly in a range of 27 to 117-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a b1003-protein, or if the activity of the polypeptide B1003, preferably represented by SEQ ID NO. 7942, or a homolog or fragment thereof, or if the activity of a polypeptide encoded by a nucleic acid molecule comprising the nucleic acid SEQ ID NO. 7941, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 7941 or polypeptide SEQ ID NO. 7942, respectively, or a homolog or a fragment thereof, is increased or generated, or if the activity b1003-protein is increased or generated in a non-human organism, like a microorganism or a plant cell, plant or part thereof, especially with non-targeted localization, For example, an increase of the threonine of at least 1 percent, particularly in a range of 60 to 144-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a b1108-protein, or if the activity of the polypeptide B1108, preferably represented by SEQ ID NO. 36938, or a homolog or fragment thereof, or if the activity of a polypeptide encoded by a nucleic acid molecule comprising the nucleic acid SEQ ID NO. 36937, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 36937 or polypeptide SEQ ID NO. 36938, respectively, or a homolog or a fragment thereof, is increased or generated, or if the activity b1108-protein is increased or generated in a non-human organism, like a microorganism or a plant cell, plant or part thereof, especially with non-targeted localization, For example, an increase of the threonine of at least 1 percent, particularly in a range of 17 to 59-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a restriction alleviation protein, or if the activity of the polypeptide B1348, preferably represented by SEQ ID NO. 77769, or a homolog or fragment thereof, or if the activity of a polypeptide encoded by a nucleic acid molecule comprising the nucleic acid SEQ ID NO. 77768, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 77768 or polypeptide SEQ ID NO. 77769, respectively, or a homolog or a fragment thereof, is increased or generated, or if the activity restriction alleviation protein is increased or generated in a non-human organism, like a microorganism or a plant cell, plant or part thereof, especially with non-targeted localization, For example, an increase of the threonine of at least 1 percent, particularly in a range of 32 to 412 percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a b1445-protein, or if the activity of the polypeptide B1445, preferably represented by SEQ ID NO. 38290, or a homolog or fragment thereof, or if the activity of a polypeptide encoded by a nucleic acid molecule comprising the nucleic acid SEQ ID NO. 38289, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 38289 or polypeptide SEQ ID NO. 38290, respectively, or a homolog or a fragment thereof, is increased or generated, or if the activity b1445-protein is increased or generated in a non-human organism, like a microorganism or a plant cell, plant or part thereof, especially with non-targeted localization, For example, an increase of the threonine of at least 1 percent, particularly in a range of 34 to 72-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a NADP-dependent malic enzyme, or if the activity of the polypeptide B1479, preferably represented by SEQ ID NO. 77775, or a homolog or fragment thereof, or if the activity of a polypeptide encoded by a nucleic acid molecule comprising the nucleic acid SEQ ID NO. 77774, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 77774 or polypeptide SEQ ID NO. 77775, respectively, or a homolog or a fragment thereof, is increased or generated, or if the activity NADP-dependent malic enzyme is increased or generated in a non-human organism, like a microorganism or a plant cell, plant or part thereof, especially with non-targeted localization, For example, an increase of the threonine of at least 1 percent, particularly in a range of 71 to 160-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a b1522-protein, or if the activity of the polypeptide B1522, preferably represented by SEQ ID NO. 7948, or a homolog or fragment thereof, or if the activity of a polypeptide encoded by a nucleic acid molecule comprising the nucleic acid SEQ ID NO. 7947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 7947 or polypeptide SEQ ID NO. 7948, respectively, or a homolog or a fragment thereof, is increased or generated, or if the activity b1522-protein is increased or generated in a non-human organism, like a microorganism or a plant cell, plant or part thereof, especially with non-targeted localization, For example, an increase of the threonine of at least 1 percent, particularly in a range of 30 to 93-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a b1670-protein, or if the activity of the polypeptide B1670, preferably represented by SEQ ID NO. 78754, or a homolog or fragment thereof, or if the activity of a polypeptide encoded by a nucleic acid molecule comprising the nucleic acid SEQ ID NO. 78753, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 78753 or polypeptide SEQ ID NO. 78754, respectively, or a homolog or a fragment thereof, is increased or generated, or if the activity b1670-protein is increased or generated in a non-human organism, like a microorganism or a plant cell, plant or part thereof, especially with non-targeted localization, For example, an increase of the threonine of at least 1 percent, particularly in a range of 18 to 47-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a pyruvate kinase, or if the activity of the polypeptide B1854, preferably represented by SEQ ID NO. 8364, or a homolog or fragment thereof, or if the activity of a polypeptide encoded by a nucleic acid molecule comprising the nucleic acid SEQ ID NO. 8363, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 8363 or polypeptide SEQ ID NO. 8364, respectively, or a homolog or a fragment thereof, is increased or generated, or if the activity pyruvate kinase is increased or generated in a non-human organism, like a microorganism or a plant cell, plant or part thereof, especially with plastidic localization, For example, an increase of the threonine of at least 1 percent, particularly in a range of 28 to 121-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a b1930-protein, or if the activity of the polypeptide B1930, preferably represented by SEQ ID NO. 79182, or a homolog or fragment thereof, or if the activity of a polypeptide encoded by a nucleic acid molecule comprising the nucleic acid SEQ ID NO. 79181, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 79181 or polypeptide SEQ ID NO. 79182, respectively, or a homolog or a fragment thereof, is increased or generated, or if the activity b1930-protein is increased or generated in a non-human organism, like a microorganism or a plant cell, plant or part thereof, especially with non-targeted localization, For example, an increase of the threonine of at least 1 percent, particularly in a range of 16 to 77-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a transport protein, or if the activity of the polypeptide B2063, preferably represented by SEQ ID NO. 38948, or a homolog or fragment thereof, or if the activity of a polypeptide encoded by a nucleic acid molecule comprising the nucleic acid SEQ ID NO. 38947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 38947 or polypeptide SEQ ID NO. 38948, respectively, or a homolog or a fragment thereof, is increased or generated, or if the activity transport protein is increased or generated in a non-human organism, like a microorganism or a plant cell, plant or part thereof, especially with non-targeted localization, For example, an increase of the threonine of at least 1 percent, particularly in a range of 17 to 157-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a b2360-protein, or if the activity of the polypeptide B2360, preferably represented by SEQ ID NO. 39220, or a homolog or fragment thereof, or if the activity of a polypeptide encoded by a nucleic acid molecule comprising the nucleic acid SEQ ID NO. 39219, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 39219 or polypeptide SEQ ID NO. 39220, respectively, or a homolog or a fragment thereof, is increased or generated, or if the activity B2360-protein is increased or generated in a non-human organism, like a microorganism or a plant cell, plant or part thereof, especially with non-targeted localization, For example, an increase of the threonine of at least 1 percent, particularly in a range of 21 to 27-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a D-serine dehydratase, or if the activity of the polypeptide B2366, preferably represented by SEQ ID NO. 79343, or a homolog or fragment thereof, or if the activity of a polypeptide encoded by a nucleic acid molecule comprising the nucleic acid SEQ ID NO. 79342, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 79342 or polypeptide SEQ ID NO. 79343, respectively, or a homolog or a fragment thereof, is increased or generated, or if the activity D-serine dehydratase is increased or generated in a non-human organism, like a microorganism or a plant cell, plant or part thereof, especially with plastidic localization, For example, an increase of the threonine of at least 1 percent, particularly in a range of 19 to 81-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a b2399-protein, or if the activity of the polypeptide B2399, preferably represented by SEQ ID NO. 39238, or a homolog or fragment thereof, or if the activity of a polypeptide encoded by a nucleic acid molecule comprising the nucleic acid SEQ ID NO. 39237, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 39237 or polypeptide SEQ ID NO. 39238, respectively, or a homolog or a fragment thereof, is increased or generated, or if the activity b2399-protein is increased or generated in a non-human organism, like a microorganism or a plant cell, plant or part thereof, especially with non-targeted localization, For example, an increase of the threonine of at least 1 percent, particularly in a range of 29 to 74-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a transcriptional regulator, or if the activity of the polypeptide B2405, preferably represented by SEQ ID NO. 39256, or a homolog or fragment thereof, or if the activity of a polypeptide encoded by a nucleic acid molecule comprising the nucleic acid SEQ ID NO. 39255, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 39255 or polypeptide SEQ ID NO. 39256, respectively, or a homolog or a fragment thereof, is increased or generated, or if the activity transcriptional regulator is increased or generated in a non-human organism, like a microorganism or a plant cell, plant or part thereof, especially with non-targeted localization, For example, an increase of the threonine of at least 1 percent, particularly in a range of 20 to 28-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a b2513-protein, or if the activity of the polypeptide B2513, preferably represented by SEQ ID NO. 9168, or a homolog or fragment thereof, or if the activity of a polypeptide encoded by a nucleic acid molecule comprising the nucleic acid SEQ ID NO. 9167, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 9167 or polypeptide SEQ ID NO. 9168, respectively, or a homolog or a fragment thereof, is increased or generated, or if the activity b2513-protein is increased or generated in a non-human organism, like a microorganism or a plant cell, plant or part thereof, especially with non-targeted localization, For example, an increase of the threonine of at least 1 percent, particularly in a range of 38 to 74-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a b2613-protein, or if the activity of the polypeptide B2613, preferably represented by SEQ ID NO. 40666, or a homolog or fragment thereof, or if the activity of a polypeptide encoded by a nucleic acid molecule comprising the nucleic acid SEQ ID NO. 40665, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 40665 or polypeptide SEQ ID NO. 40666, respectively, or a homolog or a fragment thereof, is increased or generated, or if the activity b2613-protein is increased or generated in a non-human organism, like a microorganism or a plant cell, plant or part thereof, especially with non-targeted localization, For example, an increase of the threonine of at least 1 percent, particularly in a range of 19 to 54-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a murein transglycosylase, or if the activity of the polypeptide B2701, preferably represented by SEQ ID NO. 80757, or a homolog or fragment thereof, or if the activity of a polypeptide encoded by a nucleic acid molecule comprising the nucleic acid SEQ ID NO. 80756, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 80756 or polypeptide SEQ ID NO. 80757, respectively, or a homolog or a fragment thereof, is increased or generated, or if the activity murein transglycosylase is increased or generated in a non-human organism, like a microorganism or a plant cell, plant or part thereof, especially with non-targeted localization, For example, an increase of the threonine of at least 1 percent, particularly in a range of 30 to 59-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a transcriptional regulator, or if the activity of the polypeptide B2714, preferably represented by SEQ ID NO. 40742, or a homolog or fragment thereof, or if the activity of a polypeptide encoded by a nucleic acid molecule comprising the nucleic acid SEQ ID NO. 40741, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 40741 or polypeptide SEQ ID NO. 40742, respectively, or a homolog or a fragment thereof, is increased or generated, or if the activity transcriptional regulator is increased or generated in a non-human organism, like a microorganism or a plant cell, plant or part thereof, especially with non-targeted localization, For example, an increase of the threonine of at least 1 percent, particularly in a range of 17 to 68-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a b2739-protein, or if the activity of the polypeptide B2739, preferably represented by SEQ ID NO. 80907, or a homolog or fragment thereof, or if the activity of a polypeptide encoded by a nucleic acid molecule comprising the nucleic acid SEQ ID NO. 80906, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 80906 or polypeptide SEQ ID NO. 80907, respectively, or a homolog or a fragment thereof, is increased or generated, or if the activity b2739-protein is increased or generated in a non-human organism, like a microorganism or a plant cell, plant or part thereof, especially with non-targeted localization, For example, an increase of the threonine of at least 1 percent, particularly in a range of 28 to 74-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a 2-C-methyl-D-erythritol 4-phosphate cytidylyltransferase, or if the activity of the polypeptide B2747, preferably represented by SEQ ID NO. 81076, or a homolog or fragment thereof, or if the activity of a polypeptide encoded by a nucleic acid molecule comprising the nucleic acid SEQ ID NO. 81075, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 81075 or polypeptide SEQ ID NO. 81076, respectively, or a homolog or a fragment thereof, is increased or generated, or if the activity 2-C-methyl-D-erythritol 4-phosphate cytidylyltransferase is increased or generated in a non-human organism, like a microorganism or a plant cell, plant or part thereof, especially with non-targeted localization, For example, an increase of the threonine of at least 1 percent, particularly in a range of 29 to 59-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a b2812-protein, or if the activity of the polypeptide B2812, preferably represented by SEQ ID NO. 40796, or a homolog or fragment thereof, or if the activity of a polypeptide encoded by a nucleic acid molecule comprising the nucleic acid SEQ ID NO. 40795, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 40795 or polypeptide SEQ ID NO. 40796, respectively, or a homolog or a fragment thereof, is increased or generated, or if the activity b2812-protein is increased or generated in a non-human organism, like a microorganism or a plant cell, plant or part thereof, especially with plastidic localization, For example, an increase of the threonine of at least 1 percent, particularly in a range of 23 to 44-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a b2812-protein, or if the activity of the polypeptide B2812, preferably represented by SEQ ID NO. 40796, or a homolog or fragment thereof, or if the activity of a polypeptide encoded by a nucleic acid molecule comprising the nucleic acid SEQ ID NO. 40795, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 40795 or polypeptide SEQ ID NO. 40796, respectively, or a homolog or a fragment thereof, is increased or generated, or if the activity b2812-protein is increased or generated in a non-human organism, like a microorganism or a plant cell, plant or part thereof, especially with non-targeted localization, For example, an increase of the threonine of at least 1 percent, particularly in a range of 28 to 59-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a D-3-phosphoglycerate dehydrogenase, or if the activity of the polypeptide B2913, preferably represented by SEQ ID NO. 81989, or a homolog or fragment thereof, or if the activity of a polypeptide encoded by a nucleic acid molecule comprising the nucleic acid SEQ ID NO. 81988, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 81988 or polypeptide SEQ ID NO. 81989, respectively, or a homolog or a fragment thereof, is increased or generated, or if the activity D-3-phosphoglycerate dehydrogenase is increased or generated in a non-human organism, like a microorganism or a plant cell, plant or part thereof, especially with plastidic localization, For example, an increase of the threonine of at least 1 percent, particularly in a range of 26 to 200-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a L-asparaginase, or if the activity of the polypeptide B2957, preferably represented by SEQ ID NO. 41500, or a homolog or fragment thereof, or if the activity of a polypeptide encoded by a nucleic acid molecule comprising the nucleic acid SEQ ID NO. 41499, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 41499 or polypeptide SEQ ID NO. 41500, respectively, or a homolog or a fragment thereof, is increased or generated, or if the activity L-asparaginase is increased or generated in a non-human organism, like a microorganism or a plant cell, plant or part thereof, especially with non-targeted localization, For example, an increase of the threonine of at least 1 percent, particularly in a range of 18 to 72-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a glycoprotease, or if the activity of the polypeptide B3064, preferably represented by SEQ ID NO. 42047, or a homolog or fragment thereof, or if the activity of a polypeptide encoded by a nucleic acid molecule comprising the nucleic acid SEQ ID NO. 42046, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 42046 or polypeptide SEQ ID NO. 42047, respectively, or a homolog or a fragment thereof, is increased or generated, or if the activity glycoprotease is increased or generated in a non-human organism, like a microorganism or a plant cell, plant or part thereof, especially with non-targeted localization, For example, an increase of the threonine of at least 1 percent, particularly in a range of 23 to 76-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a b3083-protein, or if the activity of the polypeptide B3083, preferably represented by SEQ ID NO. 82425, or a homolog or fragment thereof, or if the activity of a polypeptide encoded by a nucleic acid molecule comprising the nucleic acid SEQ ID NO. 82424, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 82424 or polypeptide SEQ ID NO. 82425, respectively, or a homolog or a fragment thereof, is increased or generated, or if the activity b3083-protein is increased or generated in a non-human organism, like a microorganism or a plant cell, plant or part thereof, especially with non-targeted localization, For example, an increase of the threonine of at least 1 percent, particularly in a range of 26 to 46-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a methyltransferase, or if the activity of the polypeptide B3262, preferably represented by SEQ ID NO. 42503, or a homolog or fragment thereof, or if the activity of a polypeptide encoded by a nucleic acid molecule comprising the nucleic acid SEQ ID NO. 42502, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 42502 or polypeptide SEQ ID NO. 42503, respectively, or a homolog or a fragment thereof, is increased or generated, or if the activity methyltransferase is increased or generated in a non-human organism, like a microorganism or a plant cell, plant or part thereof, especially with non-targeted localization, For example, an increase of the threonine of at least 1 percent, particularly in a range of 28 to 141-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a glycogen synthase, or if the activity of the polypeptide B3429, preferably represented by SEQ ID NO. 94052, or a homolog or fragment thereof, or if the activity of a polypeptide encoded by a nucleic acid molecule comprising the nucleic acid SEQ ID NO. 94051, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 94051 or polypeptide SEQ ID NO. 94052, respectively, or a homolog or a fragment thereof, is increased or generated, or if the activity glycogen synthase is increased or generated in a non-human organism, like a microorganism or a plant cell, plant or part thereof, especially with mitochondrial localization, For example, an increase of the threonine of at least 1 percent, particularly in a range of 27 to 60-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a b3442-protein, or if the activity of the polypeptide B3442, preferably represented by SEQ ID NO. 94381, or a homolog or fragment thereof, or if the activity of a polypeptide encoded by a nucleic acid molecule comprising the nucleic acid SEQ ID NO. 94380, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 94380 or polypeptide SEQ ID NO. 94381, respectively, or a homolog or a fragment thereof, is increased or generated, or if the activity b3442-protein is increased or generated in a non-human organism, like a microorganism or a plant cell, plant or part thereof, especially with non-targeted localization, For example, an increase of the threonine of at least 1 percent, particularly in a range of 33 to 44-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a valine-pyruvate transaminase, or if the activity of the polypeptide B3572, preferably represented by SEQ ID NO. 10173, or a homolog or fragment thereof, or if the activity of a polypeptide encoded by a nucleic acid molecule comprising the nucleic acid SEQ ID NO. 10172, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 10172 or polypeptide SEQ ID NO. 10173, respectively, or a homolog or a fragment thereof, is increased or generated, or if the activity valine-pyruvate transaminase is increased or generated in a non-human organism, like a microorganism or a plant cell, plant or part thereof, especially with plastidic localization, For example, an increase of the threonine of at least 1 percent, particularly in a range of 24 to 80 percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a b3646-protein, or if the activity of the polypeptide B3646, preferably represented by SEQ ID NO. 83430, or a homolog or fragment thereof, or if the activity of a polypeptide encoded by a nucleic acid molecule comprising the nucleic acid SEQ ID NO. 83429, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 83429 or polypeptide SEQ ID NO. 83430, respectively, or a homolog or a fragment thereof, is increased or generated, or if the activity b3646-protein is increased or generated in a non-human organism, like a microorganism or a plant cell, plant or part thereof, especially with non-targeted localization, For example, an increase of the threonine of at least 1 percent, particularly in a range of 24 to 35-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a ATP synthase subunit beta, or if the activity of the polypeptide B3732, preferably represented by SEQ ID NO. 43249, or a homolog or fragment thereof, or if the activity of a polypeptide encoded by a nucleic acid molecule comprising the nucleic acid SEQ ID NO. 43248, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 43248 or polypeptide SEQ ID NO. 43249, respectively, or a homolog or a fragment thereof, is increased or generated, or if the activity ATP synthase subunit beta is increased or generated in a non-human organism, like a microorganism or a plant cell, plant or part thereof, especially with non-targeted localization, For example, an increase of the threonine of at least 1 percent, particularly in a range of 17 to 37-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a dihydroxyacid dehydratase, or if the activity of the polypeptide B3771, preferably represented by SEQ ID NO. 10253, or a homolog or fragment thereof, or if the activity of a polypeptide encoded by a nucleic acid molecule comprising the nucleic acid SEQ ID NO. 10252, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 10252 or polypeptide SEQ ID NO. 10253, respectively, or a homolog or a fragment thereof, is increased or generated, or if the activity dihydroxyacid dehydratase is increased or generated in a non-human organism, like a microorganism or a plant cell, plant or part thereof, especially with non-targeted localization, For example, an increase of the threonine of at least 1 percent, particularly in a range of 26 to 72 percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a ketol-acid reductoisomerase, or if the activity of the polypeptide B3774, preferably represented by SEQ ID NO. 94387, or a homolog or fragment thereof, or if the activity of a polypeptide encoded by a nucleic acid molecule comprising the nucleic acid SEQ ID NO. 94386, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 94386 or polypeptide SEQ ID NO. 94387, respectively, or a homolog or a fragment thereof, is increased or generated, or if the activity ketol-acid reductoisomerase is increased or generated in a non-human organism, like a microorganism or a plant cell, plant or part thereof, especially with non-targeted localization, For example, an increase of the threonine of at least 1 percent, particularly in a range of 29 to 91-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a enterobacterial common antigen polymerase, or if the activity of the polypeptide B3793, preferably represented by SEQ ID NO. 43801, or a homolog or fragment thereof, or if the activity of a polypeptide encoded by a nucleic acid molecule comprising the nucleic acid SEQ ID NO. 43800, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 43800 or polypeptide SEQ ID NO. 43801, respectively, or a homolog or a fragment thereof, is increased or generated, or if the activity enterobacterial common antigen polymerase is increased or generated in a non-human organism, like a microorganism or a plant cell, plant or part thereof, especially with non-targeted localization, For example, an increase of the threonine of at least 1 percent, particularly in a range of 17 to 70-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a DNA helicase II, or if the activity of the polypeptide B3813, preferably represented by SEQ ID NO. 43840, or a homolog or fragment thereof, or if the activity of a polypeptide encoded by a nucleic acid molecule comprising the nucleic acid SEQ ID NO. 43839, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 43839 or polypeptide SEQ ID NO. 43840, respectively, or a homolog or a fragment thereof, is increased or generated, or if the activity DNA helicase II is increased or generated in a non-human organism, like a microorganism or a plant cell, plant or part thereof, especially with non-targeted localization, For example, an increase of the threonine of at least 1 percent, particularly in a range of 25 to 94-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a transcriptional regulator, or if the activity of the polypeptide B3872, preferably represented by SEQ ID NO. 83822, or a homolog or fragment thereof, or if the activity of a polypeptide encoded by a nucleic acid molecule comprising the nucleic acid SEQ ID NO. 83821, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 83821 or polypeptide SEQ ID NO. 83822, respectively, or a homolog or a fragment thereof, is increased or generated, or if the activity transcriptional regulator is increased or generated in a non-human organism, like a microorganism or a plant cell, plant or part thereof, especially with non-targeted localization, For example, an increase of the threonine of at least 1 percent, particularly in a range of 17 to 51 percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a b3989-protein, or if the activity of the polypeptide B3989, preferably represented by SEQ ID NO. 44373, or a homolog or fragment thereof, or if the activity of a polypeptide encoded by a nucleic acid molecule comprising the nucleic acid SEQ ID NO. 44372, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 44372 or polypeptide SEQ ID NO. 44373, respectively, or a homolog or a fragment thereof, is increased or generated, or if the activity b3989-protein is increased or generated in a non-human organism, like a microorganism or a plant cell, plant or part thereof, especially with non-targeted localization, For example, an increase of the threonine of at least 1 percent, particularly in a range of 22 to 68-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a aspartate kinase, or if the activity of the polypeptide B4024, preferably represented by SEQ ID NO. 83836, or a homolog or fragment thereof, or if the activity of a polypeptide encoded by a nucleic acid molecule comprising the nucleic acid SEQ ID NO. 83835, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 83835 or polypeptide SEQ ID NO. 83836, respectively, or a homolog or a fragment thereof, is increased or generated, or if the activity aspartate kinase is increased or generated in a non-human organism, like a microorganism or a plant cell, plant or part thereof, especially with plastidic localization, For example, an increase of the threonine of at least 1 percent, particularly in a range of 64 to 1969-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a b4029-protein, or if the activity of the polypeptide B4029, preferably represented by SEQ ID NO. 10741, or a homolog or fragment thereof, or if the activity of a polypeptide encoded by a nucleic acid molecule comprising the nucleic acid SEQ ID NO. 10740, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 10740 or polypeptide SEQ ID NO. 10741, respectively, or a homolog or a fragment thereof, is increased or generated, or if the activity b4029-protein is increased or generated in a non-human organism, like a microorganism or a plant cell, plant or part thereof, especially with non-targeted localization, For example, an increase of the threonine of at least 1 percent, particularly in a range of 21 to 213-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a b4056-protein, or if the activity of the polypeptide B4056, preferably represented by SEQ ID NO. 94543, or a homolog or fragment thereof, or if the activity of a polypeptide encoded by a nucleic acid molecule comprising the nucleic acid SEQ ID NO. 94542, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 94542 or polypeptide SEQ ID NO. 94543, respectively, or a homolog or a fragment thereof, is increased or generated, or if the activity b4056-protein is increased or generated in a non-human organism, like a microorganism or a plant cell, plant or part thereof, especially with non-targeted localization, For example, an increase of the threonine of at least 1 percent, particularly in a range of 18 to 43-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a b4250-protein, or if the activity of the polypeptide B4250, preferably represented by SEQ ID NO. 94736, or a homolog or fragment thereof, or if the activity of a polypeptide encoded by a nucleic acid molecule comprising the nucleic acid SEQ ID NO. 94735, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 94735 or polypeptide SEQ ID NO. 94736, respectively, or a homolog or a fragment thereof, is increased or generated, or if the activity b4250-protein is increased or generated in a non-human organism, like a microorganism or a plant cell, plant or part thereof, especially with non-targeted localization, For example, an increase of the threonine of at least 1 percent, particularly in a range of 25 to 60-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a acetyltransferase, or if the activity of the polypeptide B4256, preferably represented by SEQ ID NO. 45322, or a homolog or fragment thereof, or if the activity of a polypeptide encoded by a nucleic acid molecule comprising the nucleic acid SEQ ID NO. 45321, preferably the coding region thereof, or a homolog or fragment thereof, e.g. derived from Escherichia coli, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 45321 or polypeptide SEQ ID NO. 45322, respectively, or a homolog or a fragment thereof, is increased or generated, or if the activity acetyltransferase is increased or generated in a non-human organism, like a microorganism or a plant cell, plant or part thereof, especially with non-targeted localization, For example, an increase of the threonine of at least 1 percent, particularly in a range of 20 to 95-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a glutaredoxin, or if the activity of the polypeptide GM02LC12622, preferably represented by SEQ ID NO. 10812, or a homolog or fragment thereof, or if the activity of a polypeptide encoded by a nucleic acid molecule comprising the nucleic acid SEQ ID NO. 10811, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 10811 or polypeptide SEQ ID NO. 10812, respectively, or a homolog or a fragment thereof, is increased or generated, or if the activity glutaredoxin is increased or generated in a non-human organism, like a microorganism or a plant cell, plant or part thereof, especially with non-targeted localization, For example, an increase of the threonine of at least 1 percent, particularly in a range of 20 to 83-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a transcription factor, or if the activity of the polypeptide GM02LC21368, preferably represented by SEQ ID NO. 84199, or a homolog or fragment thereof, or if the activity of a polypeptide encoded by a nucleic acid molecule comprising the nucleic acid SEQ ID NO. 84198, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 84198 or polypeptide SEQ ID NO. 84199, respectively, or a homolog or a fragment thereof, is increased or generated, or if the activity transcription factor is increased or generated in a non-human organism, like a microorganism or a plant cell, plant or part thereof, especially with non-targeted localization, For example, an increase of the threonine of at least 1 percent, particularly in a range of 30 to 112-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a cyclin D, or if the activity of the polypeptide GM02LC44512, preferably represented by SEQ ID NO. 46851, or a homolog or fragment thereof, or if the activity of a polypeptide encoded by a nucleic acid molecule comprising the nucleic acid SEQ ID NO. 46850, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 46850 or polypeptide SEQ ID NO. 46851, respectively, or a homolog or a fragment thereof, is increased or generated, or if the activity cyclin D is increased or generated in a non-human organism, like a microorganism or a plant cell, plant or part thereof, especially with non-targeted localization, For example, an increase of the threonine of at least 1 percent, particularly in a range of 24 to 60-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a heat shock protein, or if the activity of the polypeptide Sll0170, preferably represented by SEQ ID NO. 48139, or a homolog or fragment thereof, or if the activity of a polypeptide encoded by a nucleic acid molecule comprising the nucleic acid SEQ ID NO. 48138, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 48138 or polypeptide SEQ ID NO. 48139, respectively, or a homolog or a fragment thereof, is increased or generated, or if the activity heat shock protein is increased or generated in a non-human organism, like a microorganism or a plant cell, plant or part thereof, especially with non-targeted localization, For exampie, an increase of the threonine of at least 1 percent, particularly in a range of 23 to 96-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a polyphosphate kinase, or if the activity of the polypeptide Sll0290, preferably represented by SEQ ID NO. 49829, or a homolog or fragment thereof, or if the activity of a polypeptide encoded by a nucleic acid molecule comprising the nucleic acid SEQ ID NO. 49828, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 49828 or polypeptide SEQ ID NO. 49829, respectively, or a homolog or a fragment thereof, is increased or generated, or if the activity polyphosphate kinase is increased or generated in a non-human organism, like a microorganism or a plant cell, plant or part thereof, especially with non-targeted localization, For example, an increase of the threonine of at least 1 percent, particularly in a range of 17 to 53-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a branched chain amino acid ABC transporter ATP-binding protein, or if the activity of the polypeptide Sll0374, preferably represented by SEQ ID NO. 94742, or a homolog or fragment thereof, or if the activity of a polypeptide encoded by a nucleic acid molecule comprising the nucleic acid SEQ ID NO. 94741, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 94741 or polypeptide SEQ ID NO. 94742, respectively, or a homolog or a fragment thereof, is increased or generated, or if the activity branched chain amino acid ABC transporter ATP-binding protein is increased or generated in a non-human organism, like a microorganism or a plant cell, plant or part thereof, especially with plastidic localization, For example, an increase of the threonine of at least 1 percent, particularly in a range of 21 to 30-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a uroporphyrin-III C-methyltransferase, or if the activity of the polypeptide Sll0378, preferably represented by SEQ ID NO. 84870, or a homolog or fragment thereof, or if the activity of a polypeptide encoded by a nucleic acid molecule comprising the nucleic acid SEQ ID NO. 84869, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 84869 or polypeptide SEQ ID NO. 84870, respectively, or a homolog or a fragment thereof, is increased or generated, or if the activity uroporphyrin-III C-methyltransferase is increased or generated in a non-human organism, like a microorganism or a plant cell, plant or part thereof, especially with plastidic localization, For example, an increase of the threonine of at least 1 percent, particularly in a range of 19 to 83-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a sll0418-protein, or if the activity of the polypeptide Sll0418, preferably represented by SEQ ID NO. 94943, or a homolog or fragment thereof, or if the activity of a polypeptide encoded by a nucleic acid molecule comprising the nucleic acid SEQ ID NO. 94942, preferably the coding region thereof, or a homolog or fragment thereof, e.g. derived from Synechocystis sp., is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 94942 or polypeptide SEQ ID NO. 94943, respectively, or a homolog or a fragment thereof, is increased or generated, or if the activity sll0418-protein is increased or generated in a non-human organism, like a microorganism or a plant cell, plant or part thereof, especially with non-targeted localization, For example, an increase of the threonine of at least 1 percent, particularly in a range of 17 to 30-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a oxireductase, or if the activity of the polypeptide Sll0816, preferably represented by SEQ ID NO. 51199, or a homolog or fragment thereof, or if the activity of a polypeptide encoded by a nucleic acid molecule comprising the nucleic acid SEQ ID NO. 51198, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 51198 or polypeptide SEQ ID NO. 51199, respectively, or a homolog or a fragment thereof, is increased or generated, or if the activity oxireductase is increased or generated in a non-human organism, like a microorganism or a plant cell, plant or part thereof, especially with non-targeted localization, For example, an increase of the threonine of at least 1 percent, particularly in a range of 19 to 91-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a malate dehydrogenase, or if the activity of the polypeptide Sll0891, preferably represented by SEQ ID NO. 51269, or a homolog or fragment thereof, or if the activity of a polypeptide encoded by a nucleic acid molecule comprising the nucleic acid SEQ ID NO. 51268, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 51268 or polypeptide SEQ ID NO. 51269, respectively, or a homolog or a fragment thereof, is increased or generated, or if the activity malate dehydrogenase is increased or generated in a non-human organism, like a microorganism or a plant cell, plant or part thereof, especially with plastidic localization, For example, an increase of the threonine of at least 1 percent, particularly in a range of 23 to 61-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a phosphoribosylaminoimidazole carboxylase catalytic subunit, or if the activity of the polypeptide Sll0901, preferably represented by SEQ ID NO. 51633, or a homolog or fragment thereof, or if the activity of a polypeptide encoded by a nucleic acid molecule comprising the nucleic acid SEQ ID NO. 51632, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 51632 or polypeptide SEQ ID NO. 51633, respectively, or a homolog or a fragment thereof, is increased or generated, or if the activity phosphoribosylaminoimidazole carboxylase catalytic subunit is increased or generated in a non-human organism, like a microorganism or a plant cell, plant or part thereof, especially with mitochondrial localization, For example, an increase of the threonine of at least 1 percent, particularly in a range of 39 to 78-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a glycogen synthase, or if the activity of the polypeptide Sll0945, preferably represented by SEQ ID NO. 52365, or a homolog or fragment thereof, or if the activity of a polypeptide encoded by a nucleic acid molecule comprising the nucleic acid SEQ ID NO. 52364, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 52364 or polypeptide SEQ ID NO. 52365, respectively, or a homolog or a fragment thereof, is increased or generated, or if the activity glycogen synthase is increased or generated in a non-human organism, like a microorganism or a plant cell, plant or part thereof, especially with non-targeted localization, For exampie, an increase of the threonine of at least 1 percent, particularly in a range of 17 to 56-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a glycogen (starch) synthase, or if the activity of the polypeptide Sll1393, preferably represented by SEQ ID NO. 53457, or a homolog or fragment thereof, or if the activity of a polypeptide encoded by a nucleic acid molecule comprising the nucleic acid SEQ ID NO. 53456, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 53456 or polypeptide SEQ ID NO. 53457, respectively, or a homolog or a fragment thereof, is increased or generated, or if the activity glycogen (starch) synthase is increased or generated in a non-human organism, like a microorganism or a plant cell, plant or part thereof, especially with non-targeted localization, For example, an increase of the threonine of at least 1 percent, particularly in a range of 18 to 84-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a nitrate/nitrite transport protein, or if the activity of the polypeptide Sll1450, preferably represented by SEQ ID NO. 54338, or a homolog or fragment thereof, or if the activity of a polypeptide encoded by a nucleic acid molecule comprising the nucleic acid SEQ ID NO. 54337, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 54337 or polypeptide SEQ ID NO. 54338, respectively, or a homolog or a fragment thereof, is increased or generated, or if the activity nitrate/nitrite transport protein is increased or generated in a non-human organism, like a microorganism or a plant cell, plant or part thereof, especially with mitochondrial localization, For example, an increase of the threonine of at least 1 percent, particularly in a range of 17 to 40-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a carbamoylphosphate synthase subunit, or if the activity of the polypeptide Sll1498, preferably represented by SEQ ID NO. 95048, or a homolog or fragment thereof, or if the activity of a polypeptide encoded by a nucleic acid molecule comprising the nucleic acid SEQ ID NO. 95047, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 95047 or polypeptide SEQ ID NO. 95048, respectively, or a homolog or a fragment thereof, is increased or generated, or if the activity carbamoyl-phosphate synthase subunit is increased or generated in a non-human organism, like a microorganism or a plant cell, plant or part thereof, especially with plastidic localization, For example, an increase of the threonine of at least 1 percent, particularly in a range of 18 to 30-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a glutathione S-transferase, or if the activity of the polypeptide Sll1545, preferably represented by SEQ ID NO. 11424, or a homolog or fragment thereof, or if the activity of a polypeptide encoded by a nucleic acid molecule comprising the nucleic acid SEQ ID NO. 11423, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 11423 or polypeptide SEQ ID NO. 11424, respectively, or a homolog or a fragment thereof, is increased or generated, or if the activity glutathione S-transferase is increased or generated in a non-human organism, like a microorganism or a plant cell, plant or part thereof, especially with mitochondrial localization, For example, an increase of the threonine of at least 1 percent, particularly in a range of 17 to 42-percent is conferred as compared to a corresponding non-transformed wild type non-human organism

Accordingly, in one embodiment, an increase of threonine in a non-human organism, as compared to a corresponding non-transformed wild type non-human organism, is conferred in the process of the invention, if the activity of a polypeptide showing the activity of a ABC metal ion transporter substrate-binding protein, or if the activity of the polypeptide Sll1598, preferably represented by SEQ ID NO. 85744, or a homolog or fragment thereof, or if the activity of a polypeptide encoded by a nucleic acid molecule comprising the nucleic acid SEQ ID NO. 85743, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 85743 or polypeptide SEQ ID NO. 85744, respectively, or a homolog or a fragment thereof, is increased or generated, or if the activity ABC metal ion transporter substrate-binding protein is increased or generated in a non-human organism, like a microorganism or a plant cell, plant or part thereof, especially with mitochondrial localization, For example, an increase of the threonine of at least 1 percent, particularly in a range of 21 to 35-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a coproporphyrinogen III oxidase, or if the activity of the polypeptide Sll1917, preferably represented by SEQ ID NO. 11472, or a homolog or fragment thereof, or if the activity of a polypeptide encoded by a nucleic acid molecule comprising the nucleic acid SEQ ID NO. 11471, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 11471 or polypeptide SEQ ID NO. 11472, respectively, or a homolog or a fragment thereof, is increased or generated, or if the activity coproporphyrinogen III oxidase is increased or generated in a non-human organism, like a microorganism or a plant cell, plant or part thereof, especially with mitochondrial localization, For example, an increase of the threonine of at least 1 percent, particularly in a range of 18 to 67-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a ferredoxin, or if the activity of the polypeptide Slr0150, preferably represented by SEQ ID NO. 95528, or a homolog or fragment thereof, or if the activity of a polypeptide encoded by a nucleic acid molecule comprising the nucleic acid SEQ ID NO. 95527, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 95527 or polypeptide SEQ ID NO. 95528, respectively, or a homolog or a fragment thereof, is increased or generated, or if the activity ferredoxin is increased or generated in a non-human organism, like a microorganism or a plant cell, plant or part thereof, especially with mitochondrial localization, For example, an increase of the threonine of at least 1 percent, particularly in a range of 18 to 53-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a slr0383-protein, or if the activity of the polypeptide Slr0383, preferably represented by SEQ ID NO. 95745, or a homolog or fragment thereof, or if the activity of a polypeptide encoded by a nucleic acid molecule comprising the nucleic acid SEQ ID NO. 95744, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 95744 or polypeptide SEQ ID NO. 95745, respectively, or a homolog or a fragment thereof, is increased or generated, or if the activity slr0383-protein is increased or generated in a non-human organism, like a microorganism or a plant cell, plant or part thereof, especially with non-targeted localization, For example, an increase of the threonine of at least 1 percent, particularly in a range of 18 to 35-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a bifunctional purine biosynthesis protein, or if the activity of the polypeptide Slr0597, preferably represented by SEQ ID NO. 56154, or a homolog or fragment thereof, or if the activity of a polypeptide encoded by a nucleic acid molecule comprising the nucleic acid SEQ ID NO. 56153, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 56153 or polypeptide SEQ ID NO. 56154, respectively, or a homolog or a fragment thereof, is increased or generated, or if the activity bifunctional purine biosynthesis protein is increased or generated in a non-human organism, like a microorganism or a plant cell, plant or part thereof, especially with plastidic localization, For example, an increase of the threonine of at least 1 percent, particularly in a range of 32 to 35-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a aspartate kinase, or if the activity of the polypeptide Slr0657, preferably represented by SEQ ID NO. 95858, or a homolog or fragment thereof, or if the activity of a polypeptide encoded by a nucleic acid molecule comprising the nucleic acid SEQ ID NO. 95857, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 95857 or polypeptide SEQ ID NO. 95858, respectively, or a homolog or a fragment thereof, is increased or generated, or if the activity aspartate kinase is increased or generated in a non-human organism, like a microorganism or a plant cell, plant or part thereof, especially with mitochondrial localization, For example, an increase of the threonine of at least 1 percent, particularly in a range of 20 to 46-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a geranylgeranyl pyrophosphate synthase, or if the activity of the polypeptide Slr0739, preferably represented by SEQ ID NO. 57236, or a homolog or fragment thereof, or if the activity of a polypeptide encoded by a nucleic acid molecule comprising the nucleic acid SEQ ID NO. 57235, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 57235 or polypeptide SEQ ID NO. 57236, respectively, or a homolog or a fragment thereof, is increased or generated, or if the activity geranylgeranyl pyrophosphate synthase is increased or generated in a non-human organism, like a microorganism or a plant cell, plant or part thereof, especially with plastidic localization, For example, an increase of the threonine of at least 1 percent, particularly in a range of 16 to 79-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a kinase, or if the activity of the polypeptide Slr0862, preferably represented by SEQ ID NO. 87611, or a homolog or fragment thereof, or if the activity of a polypeptide encoded by a nucleic acid molecule comprising the nucleic acid SEQ ID NO. 87610, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 87610 or polypeptide SEQ ID NO. 87611, respectively, or a homolog or a fragment thereof, is increased or generated, or if the activity kinase is increased or generated in a non-human organism, like a microorganism or a plant cell, plant or part thereof, especially with mitochondrial localization, For example, an increase of the threonine of at least 1 percent, particularly in a range of 39 to 45-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a argininosuccinate lyase, or if the activity of the polypeptide Slr1133, preferably represented by SEQ ID NO. 87854, or a homolog or fragment thereof, or if the activity of a polypeptide encoded by a nucleic acid molecule comprising the nucleic acid SEQ ID NO. 87853, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 87853 or polypeptide SEQ ID NO. 87854, respectively, or a homolog or a fragment thereof, is increased or generated, or if the activity argininosuccinate lyase is increased or generated in a non-human organism, like a microorganism or a plant cell, plant or part thereof, especially with mitochondrial localization, For example, an increase of the threonine of at least 1 percent, particularly in a range of 23 to 34-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a photosystem II protein, or if the activity of the polypeptide Slr1739, preferably represented by SEQ ID NO. 58732, or a homolog or fragment thereof, or if the activity of a polypeptide encoded by a nucleic acid molecule comprising the nucleic acid SEQ ID NO. 58731, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 58731 or polypeptide SEQ ID NO. 58732, respectively, or a homolog or a fragment thereof, is increased or generated, or if the activity photosystem II protein is increased or generated in a non-human organism, like a microorganism or a plant cell, plant or part thereof, especially with plastidic localization, For example, an increase of the threonine of at least 1 percent, particularly in a range of 27 to 512-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a cell division protein, or if the activity of the polypeptide TTC0035, preferably represented by SEQ ID NO. 60302, or a homolog or fragment thereof, or if the activity of a polypeptide encoded by a nucleic acid molecule comprising the nucleic acid SEQ ID NO. 60301, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 60301 or polypeptide SEQ ID NO. 60302, respectively, or a homolog or a fragment thereof, is increased or generated, or if the activity cell division protein is increased or generated in a non-human organism, like a microorganism or a plant cell, plant or part thereof, especially with non-targeted localization, For example, an increase of the threonine of at least 1 percent, particularly in a range of 32 to 102-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a branched-chain amino acid ABC transporter permease protein, or if the activity of the polypeptide TTC0216, preferably represented by SEQ ID NO. 60860, or a homolog or fragment thereof, or if the activity of a polypeptide encoded by a nucleic acid molecule comprising the nucleic acid SEQ ID NO. 60859, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 60859 or polypeptide SEQ ID NO. 60860, respectively, or a homolog or a fragment thereof, is increased or generated, or if the activity branched-chain amino acid ABC transporter permease protein is increased or generated in a non-human organism, like a microorganism or a plant cell, plant or part thereof, especially with non-targeted localization, For example, an increase of the threonine of at least 1 percent, particularly in a range of 18 to 35-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a TTC0881protein, or if the activity of the polypeptide TTC0881, preferably represented by SEQ ID NO. 88876, or a homolog or fragment thereof, or if the activity of a polypeptide encoded by a nucleic acid molecule comprising the nucleic acid SEQ ID NO. 88875, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 88875 or polypeptide SEQ ID NO. 88876, respectively, or a homolog or a fragment thereof, is increased or generated, or if the activity TTC0881-protein is increased or generated in a non-human organism, like a microorganism or a plant cell, plant or part thereof, especially with non-targeted localization, For example, an increase of the threonine of at least 1 percent, particularly in a range of 19 to 34-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a metal-dependent hydrolase, or if the activity of the polypeptide TTC0917, preferably represented by SEQ ID NO. 61554, or a homolog or fragment thereof, or if the activity of a polypeptide encoded by a nucleic acid molecule comprising the nucleic acid SEQ ID NO. 61553, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 61553 or polypeptide SEQ ID NO. 61554, respectively, or a homolog or a fragment thereof, is increased or generated, or if the activity metal-dependent hydrolase is increased or generated in a non-human organism, like a microorganism or a plant cell, plant or part thereof, especially with non-targeted localization, For example, an increase of the threonine of at least 1 percent, particularly in a range of 18 to 26-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a homocitrate synthase, or if the activity of the polypeptide TTC1550, preferably represented by SEQ ID NO. 12975, or a homolog or fragment thereof, or if the activity of a polypeptide encoded by a nucleic acid molecule comprising the nucleic acid SEQ ID NO. 12974, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 12974 or polypeptide SEQ ID NO. 12975, respectively, or a homolog or a fragment thereof, is increased or generated, or if the activity homocitrate synthase is increased or generated in a non-human organism, like a microorganism or a plant cell, plant or part thereof, especially with non-targeted localization, For example, an increase of the threonine of at least 1 percent, particularly in a range of 32 to 94-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a XM_{—}473199-protein, or if the activity of the polypeptide XM_{—}473199, preferably represented by SEQ ID NO. 62245, or a homolog or fragment thereof, or if the activity of a polypeptide encoded by a nucleic acid molecule comprising the nucleic acid SEQ ID NO. 62244, preferably the coding region thereof, or a homolog or fragment thereof, e.g. derived from Oryza sativa, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 62244 or polypeptide SEQ ID NO. 62245, respectively, or a homolog or a fragment thereof, is increased or generated, or if the activity XM_{—}473199-protein is increased or generated in a non-human organism, like a microorganism or a plant cell, plant or part thereof, especially with non-targeted localization, For example, an increase of the threonine of at least 1 percent, particularly in a range of 25 to 82-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a pyruvate kinase, or if the activity of the polypeptide Yal038w, preferably represented by SEQ ID NO. 96023, or a homolog or fragment thereof, or if the activity of a polypeptide encoded by a nucleic acid molecule comprising the nucleic acid SEQ ID NO. 96022, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 96022 or polypeptide SEQ ID NO. 96023, respectively, or a homolog or a fragment thereof, is increased or generated, or if the activity pyruvate kinase is increased or generated in a non-human organism, like a microorganism or a plant cell, plant or part thereof, especially with plastidic localization, For example, an increase of the threonine of at least 1 percent, particularly in a range of 23 to 101-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a transcription factor, or if the activity of the polypeptide Ybl021 c, preferably represented by SEQ ID NO. 62525, or a homolog or fragment thereof, or if the activity of a polypeptide encoded by a nucleic acid molecule comprising the nucleic acid SEQ ID NO. 62524, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 62524 or polypeptide SEQ ID NO. 62525, respectively, or a homolog or a fragment thereof, is increased or generated, or if the activity transcription factor is increased or generated in a non-human organism, like a microorganism or a plant cell, plant or part thereof, especially with non-targeted localization, For example, an increase of the threonine of at least 1 percent, particularly in a range of 17 to 83-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a cell division control protein, or if the activity of the polypeptide Ybr160w, preferably represented by SEQ ID NO. 62718, or a homolog or fragment thereof, or if the activity of a polypeptide encoded by a nucleic acid molecule comprising the nucleic acid SEQ ID NO. 62717, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 62717 or polypeptide SEQ ID NO. 62718, respectively, or a homolog or a fragment thereof, is increased or generated, or if the activity cell division control protein is increased or generated in a non-human organism, like a microorganism or a plant cell, plant or part thereof, especially with non-targeted localization, For example, an increase of the threonine of at least 1 percent, particularly in a range of 25 to 60-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a pyruvate carboxylase, or if the activity of the polypeptide Ybr218c, preferably represented by SEQ ID NO. 96780, or a homolog or fragment thereof, or if the activity of a polypeptide encoded by a nucleic acid molecule comprising the nucleic acid SEQ ID NO. 96779, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 96779 or polypeptide SEQ ID NO. 96780, respectively, or a homolog or a fragment thereof, is increased or generated, or if the activity pyruvate carboxylase is increased or generated in a non-human organism, like a microorganism or a plant cell, plant or part thereof, especially with plastidic localization, For example, an increase of the threonine of at least 1 percent, particularly in a range of 17 to 27-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a ycl027w-protein, or if the activity of the polypeptide Ycl027w, preferably represented by SEQ ID NO. 96981, or a homolog or fragment thereof, or if the activity of a polypeptide encoded by a nucleic acid molecule comprising the nucleic acid SEQ ID NO. 96980, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 96980 or polypeptide SEQ ID NO. 96981, respectively, or a homolog or a fragment thereof, is increased or generated, or if the activity ycl027w-protein is increased or generated in a non-human organism, like a microorganism or a plant cell, plant or part thereof, especially with plastidic localization, For example, an increase of the threonine of at least 1 percent, particularly in a range of 19 to 26-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a coproporphyrinogen III oxidase, or if the activity of the polypeptide Ydr044w, preferably represented by SEQ ID NO. 63335, or a homolog or fragment thereof, or if the activity of a polypeptide encoded by a nucleic acid molecule comprising the nucleic acid SEQ ID NO. 63334, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 63334 or polypeptide SEQ ID NO. 63335, respectively, or a homolog or a fragment thereof, is increased or generated, or if the activity coproporphyrinogen III oxidase is increased or generated in a non-human organism, like a microorganism or a plant cell, plant or part thereof, especially with plastidic localization, For example, an increase of the threonine of at least 1 percent, particularly in a range of 74 to 194-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a ydr183w-protein, or if the activity of the polypeptide Ydr183w, preferably represented by SEQ ID NO. 90104, or a homolog or fragment thereof, or if the activity of a polypeptide encoded by a nucleic acid molecule comprising the nucleic acid SEQ ID NO. 90103, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 90103 or polypeptide SEQ ID NO. 90104, respectively, or a homolog or a fragment thereof, is increased or generated, or if the activity ydr183w-protein is increased or generated in a non-human organism, like a microorganism or a plant cell, plant or part thereof, especially with plastidic localization, For example, an increase of the threonine of at least 1 percent, particularly in a range of 17 to 53-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a hexose transporter, or if the activity of the polypeptide Ydr342c, preferably represented by SEQ ID NO. 96987, or a homolog or fragment thereof, or if the activity of a polypeptide encoded by a nucleic acid molecule comprising the nucleic acid SEQ ID NO. 96986, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 96986 or polypeptide SEQ ID NO. 96987, respectively, or a homolog or a fragment thereof, is increased or generated, or if the activity hexose transporter is increased or generated in a non-human organism, like a microorganism or a plant cell, plant or part thereof, especially with plastidic localization, For example, an increase of the threonine of at least 1 percent, particularly in a range of 19 to 92-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a YEL030C-A-protein, or if the activity of the polypeptide Yel030c-a, preferably represented by SEQ ID NO. 97295, or a homolog or fragment thereof, or if the activity of a polypeptide encoded by a nucleic acid molecule comprising the nucleic acid SEQ ID NO. 97294, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 97294 or polypeptide SEQ ID NO. 97295, respectively, or a homolog or a fragment thereof, is increased or generated, or if the activity YEL030C-A-protein is increased or generated in a non-human organism, like a microorganism or a plant cell, plant or part thereof, especially with non-targeted localization, For example, an increase of the threonine of at least 1 percent, particularly in a range of 16 to 31-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a hexose transporter, or if the activity of the polypeptide Yel069c, preferably represented by SEQ ID NO. 97299, or a homolog or fragment thereof, or if the activity of a polypeptide encoded by a nucleic acid molecule comprising the nucleic acid SEQ ID NO. 97298, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 97298 or polypeptide SEQ ID NO. 97299, respectively, or a homolog or a fragment thereof, is increased or generated, or if the activity hexose transporter is increased or generated in a non-human organism, like a microorganism or a plant cell, plant or part thereof, especially with plastidic localization, For example, an increase of the threonine of at least 1 percent, particularly in a range of 21 to 35-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a yfl054c-protein, or if the activity of the polypeptide Yfl054c, preferably represented by SEQ ID NO. 64149, or a homolog or fragment thereof, or if the activity of a polypeptide encoded by a nucleic acid molecule comprising the nucleic acid SEQ ID NO. 64148, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 64148 or polypeptide SEQ ID NO. 64149, respectively, or a homolog or a fragment thereof, is increased or generated, or if the activity yfl054c-protein is increased or generated in a non-human organism, like a microorganism or a plant cell, plant or part thereof, especially with plastidic localization, For example, an increase of the threonine of at least 1 percent, particularly in a range of 16 to 23-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a ygl237c-protein, or if the activity of the polypeptide Ygl237c, preferably represented by SEQ ID NO. 64178, or a homolog or fragment thereof, or if the activity of a polypeptide encoded by a nucleic acid molecule comprising the nucleic acid SEQ ID NO. 64177, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 64177 or polypeptide SEQ ID NO. 64178, respectively, or a homolog or a fragment thereof, is increased or generated, or if the activity ygl237c-protein is increased or generated in a non-human organism, like a microorganism or a plant cell, plant or part thereof, especially with non-targeted localization, For example, an increase of the threonine of at least 1 percent, particularly in a range of 27 to 84-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a yhr131c-protein, or if the activity of the polypeptide Yhr131c, preferably represented by SEQ ID NO. 97621, or a homolog or fragment thereof, or if the activity of a polypeptide encoded by a nucleic acid molecule comprising the nucleic acid SEQ ID NO. 97620, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 97620 or polypeptide SEQ ID NO. 97621, respectively, or a homolog or a fragment thereof, is increased or generated, or if the activity yhr131c-protein is increased or generated in a non-human organism, like a microorganism or a plant cell, plant or part thereof, especially with plastidic localization, For example, an increase of the threonine of at least 1 percent, particularly in a range of 31 to 32-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a (DL)-glycerol-3-phosphatase, or if the activity of the polypeptide Yil053w, preferably represented by SEQ ID NO. 14770, or a homolog or fragment thereof, or if the activity of a polypeptide encoded by a nucleic acid molecule comprising the nucleic acid SEQ ID NO. 14769, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 14769 or polypeptide SEQ ID NO. 14770, respectively, or a homolog or a fragment thereof, is increased or generated, or if the activity (DL)-glycerol-3-phosphatase is increased or generated in a non-human organism, like a microorganism or a plant cell, plant or part thereof, especially with plastidic localization, For example, an increase of the threonine of at least 1 percent, particularly in a range of 19 to 46-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a 3-phosphoglycerate dehydrogenase, or if the activity of the polypeptide Yil074c, preferably represented by SEQ ID NO. 64564, or a homolog or fragment thereof, or if the activity of a polypeptide encoded by a nucleic acid molecule comprising the nucleic acid SEQ ID NO. 64563, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 64563 or polypeptide SEQ ID NO. 64564, respectively, or a homolog or a fragment thereof, is increased or generated, or if the activity 3-phosphoglycerate dehydrogenase is increased or generated in a non-human organism, like a microorganism or a plant cell, plant or part thereof, especially with plastidic localization, For example, an increase of the threonine of at least 1 percent, particularly in a range of 22 to 34-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a glutathione S-transferase, or if the activity of the polypeptide Yir038c, preferably represented by SEQ ID NO. 90307, or a homolog or fragment thereof, or if the activity of a polypeptide encoded by a nucleic acid molecule comprising the nucleic acid SEQ ID NO. 90306, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 90306 or polypeptide SEQ ID NO. 90307, respectively, or a homolog or a fragment thereof, is increased or generated, or if the activity glutathione S-transferase is increased or generated in a non-human organism, like a microorganism or a plant cell, plant or part thereof, especially with plastidic localization, For example, an increase of the threonine of at least 1 percent, particularly in a range of 16 to 25-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a DnaJ-like chaperone, or if the activity of the polypeptide Yjl073w, preferably represented by SEQ ID NO. 64965, or a homolog or fragment thereof, or if the activity of a polypeptide encoded by a nucleic acid molecule comprising the nucleic acid SEQ ID NO. 64964, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 64964 or polypeptide SEQ ID NO. 64965, respectively, or a homolog or a fragment thereof, is increased or generated, or if the activity DnaJ-like chaperone is increased or generated in a non-human organism, like a microorganism or a plant cell, plant or part thereof, especially with plastidic localization, For example, an increase of the threonine of at least 1 percent, particularly in a range of 24 to 27-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a YJL127W-A-protein, or if the activity of the polypeptide Yjl127w-a, preferably represented by SEQ ID NO. 97632, or a homolog or fragment thereof, or if the activity of a polypeptide encoded by a nucleic acid molecule comprising the nucleic acid SEQ ID NO. 97631, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 97631 or polypeptide SEQ ID NO. 97632, respectively, or a homolog or a fragment thereof, is increased or generated, or if the activity YJL127W-A-protein is increased or generated in a non-human organism, like a microorganism or a plant cell, plant or part thereof, especially with non-targeted localization, For example, an increase of the threonine of at least 1 percent, particularly in a range of 18 to 61-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a hexose transporter, or if the activity of the polypeptide Yjl219w, preferably represented by SEQ ID NO. 97636, or a homolog or fragment thereof, or if the activity of a polypeptide encoded by a nucleic acid molecule comprising the nucleic acid SEQ ID NO. 97635, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 97635 or polypeptide SEQ ID NO. 97636, respectively, or a homolog or a fragment thereof, is increased or generated, or if the activity hexose transporter is increased or generated in a non-human organism, like a microorganism or a plant cell, plant or part thereof, especially with non-targeted localization, For example, an increase of the threonine of at least 1 percent, particularly in a range of 27 to 157-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a homoserine dehydrogenase, or if the activity of the polypeptide Yjr139c, preferably represented by SEQ ID NO. 14886, or a homolog or fragment thereof, or if the activity of a polypeptide encoded by a nucleic acid molecule comprising the nucleic acid SEQ ID NO. 14885, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 14885 or polypeptide SEQ ID NO. 14886, respectively, or a homolog or a fragment thereof, is increased or generated, or if the activity homoserine dehydrogenase is increased or generated in a non-human organism, like a microorganism or a plant cell, plant or part thereof, especially with plastidic localization, For example, an increase of the threonine of at least 1 percent, particularly in a range of 16 to 923-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a branched chain aminotransferase, or if the activity of the polypeptide Yjr148w, preferably represented by SEQ ID NO. 90410, or a homolog or fragment thereof, or if the activity of a polypeptide encoded by a nucleic acid molecule comprising the nucleic acid SEQ ID NO. 90409, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 90409 or polypeptide SEQ ID NO. 90410, respectively, or a homolog or a fragment thereof, is increased or generated, or if the activity branched chain aminotransferase is increased or generated in a non-human organism, like a microorganism or a plant cell, plant or part thereof, especially with plastidic localization, For example, an increase of the threonine of at least 1 percent, particularly in a range of 18 to 59-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a YKL038W-protein, or if the activity of the polypeptide YKL038W, preferably represented by SEQ ID NO. 90603, or a homolog or fragment thereof, or if the activity of a polypeptide encoded by a nucleic acid molecule comprising the nucleic acid SEQ ID NO. 90602, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 90602 or polypeptide SEQ ID NO. 90603, respectively, or a homolog or a fragment thereof, is increased or generated, or if the activity YKL038W-protein is increased or generated in a non-human organism, like a microorganism or a plant cell, plant or part thereof, especially with plastidic localization, For example, an increase of the threonine of at least 1 percent, particularly in a range of 16 to 45-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a phosphoenolpyruvate carboxykinase, or if the activity of the polypeptide Ykr097w, preferably represented by SEQ ID NO. 98076, or a homolog or fragment thereof, or if the activity of a polypeptide encoded by a nucleic acid molecule comprising the nucleic acid SEQ ID NO. 98075, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 98075 or polypeptide SEQ ID NO. 98076, respectively, or a homolog or a fragment thereof, is increased or generated, or if the activity phosphoenolpyruvate carboxykinase is increased or generated in a non-human organism, like a microorganism or a plant cell, plant or part thereof, especially with plastidic localization, For example, an increase of the threonine of at least 1 percent, partitularly in a range of 17 to 35-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a aspartate aminotransferase, or if the activity of the polypeptide Ylr027c, preferably represented by SEQ ID NO. 90616, or a homolog or fragment thereof, or if the activity of a polypeptide encoded by a nucleic acid molecule comprising the nucleic acid SEQ ID NO. 90615, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 90615 or polypeptide SEQ ID NO. 90616, respectively, or a homolog or a fragment thereof, is increased or generated, or if the activity aspartate aminotransferase is increased or generated in a non-human organism, like a microorganism or a plant cell, plant or part thereof, especially with non-targeted localization, For example, an increase of the threonine of at least 1 percent, particularly in a range of 57 to 111-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a mitochondrial dicarboxylate transporter, or if the activity of the polypeptide Ylr348c, preferably represented by SEQ ID NO. 98244, or a homolog or fragment thereof, or if the activity of a polypeptide encoded by a nucleic acid molecule comprising the nucleic acid SEQ ID NO. 98243, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 98243 or polypeptide SEQ ID NO. 98244, respectively, or a homolog or a fragment thereof, is increased or generated, or if the activity mitochondrial dicarboxylate transporter is increased or generated in a non-human organism, like a microorganism or a plant cell, plant or part thereof, especially with non-targeted localization, For example, an increase of the threonine of at least 1 percent, particularly in a range of 23 to 68-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a phosphate transporter, or if the activity of the polypeptide Yml123c, preferably represented by SEQ ID NO.

91786, or a homolog or fragment thereof, or if the activity of a polypeptide encoded by a nucleic acid molecule comprising the nucleic acid SEQ ID NO. 91785, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 91785 or polypeptide SEQ ID NO. 91786, respectively, or a homolog or a fragment thereof, is increased or generated, or if the activity phosphate transporter is increased or generated in a non-human organism, like a microorganism or a plant cell, plant or part thereof, especially with plastidic localization, For example, an increase of the threonine of at least 1 percent, particularly in a range of 22 to 60-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a protein kinase, or if the activity of the polypeptide Yol045w, preferably represented by SEQ ID NO. 67685, or a homolog or fragment thereof, or if the activity of a polypeptide encoded by a nucleic acid molecule comprising the nucleic acid SEQ ID NO. 67684, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 67684 or polypeptide SEQ ID NO. 67685, respectively, or a homolog or a fragment thereof, is increased or generated, or if the activity protein kinase is increased or generated in a non-human organism, like a microorganism or a plant cell, plant or part thereof, especially with plastidic localization, For example, an increase of the threonine of at least 1 percent, particularly in a range of 32 to 56-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a histone acetyltransferase, or if the activity of the polypeptide Yor244w, preferably represented by SEQ ID NO. 92541, or a homolog or fragment thereof, or if the activity of a polypeptide encoded by a nucleic acid molecule comprising the nucleic acid SEQ ID NO. 92540, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 92540 or polypeptide SEQ ID NO. 92541, respectively, or a homolog or a fragment thereof, is increased or generated, or if the activity histone acetyltransferase is increased or generated in a non-human organism, like a microorganism or a plant cell, plant or part thereof, especially with plastidic localization, For example, an increase of the threonine of at least 1 percent, particularly in a range of 16 to 24-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a cyclin, or if the activity of the polypeptide Ypr119w, preferably represented by SEQ ID NO. 98446, or a homolog or fragment thereof, or if the activity of a polypeptide encoded by a nucleic acid molecule comprising the nucleic acid SEQ ID NO. 98445, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 98445 or polypeptide SEQ ID NO. 98446, respectively, or a homolog or a fragment thereof, is increased or generated, or if the activity cyclin is increased or generated in a non-human organism, like a microorganism or a plant cell, plant or part thereof, especially with non-targeted localization, For example, an increase of the threonine of at least 1 percent, particularly in a range of 16 to 37-percent is conferred as compared to a corresponding non-transformed wild type non-human organism.

Accordingly, in one embodiment, an increase of threonine in a non-human organism, as compared to a corresponding non-transformed wild type non-human organism, is conferred in the process of the invention, if the activity of a polypeptide showing the activity of a Zm_{—}4842_BE510522-protein, or if the activity of the polypeptide Zm_{—}4842_BE510522, preferably represented by SEQ ID NO. 68414, or a homolog or fragment thereof, or if the activity of a polypeptide encoded by a nucleic acid molecule comprising the nucleic acid SEQ ID NO. 68413, preferably the coding region thereof, or a homolog or fragment thereof, e.g. derived from Zea mays, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 4, column 5 or 8 in the respective same line as the nucleic acid molecule SEQ ID NO. 68413 or polypeptide SEQ ID NO. 68414, respectively, or a homolog or a fragment thereof, is increased or generated, or if the activity Zm_{—}4842_BE510522-protein is increased or generated in a non-human organism, like a microorganism or a plant cell, plant or part thereof, especially with non-targeted localization, For example, an increase of the threonine of at least 1 percent, particularly in a range of 16 to 78-percent is conferred as compared to a corresponding non-transformed wild type non-human organism.

[0096.1.1.4] to [0103.1.1.4] for the disclosure of these paragraphs see [0096.1.1.1] to [0103.1.1.1] above.

In this paragraph “A” means “Goffeau et al., Science 274 (5287), 546 (1996)”, “B” means “Blattner et al., Science 277 (5331), 1453 (1997)”, “C” means “Nakamura et al., Nucleic Acids Res. 28(1), 72 (2000)”, “D” means “deposition of the sequences to the EMBL/GenBank/DDBJ databases under “Sequencing of the draft genome assembly of Azotobacter vinelandii AvOP”. in June 2005 by Copeland et al.”, “E” means “Henne et al., Nat. Biotechnol. 22 (5), 547 (2004)”, “F” means “Kaul et al., Nature 408 (6814), 796 (2000)”, and “G” means “Matsumoto et al., Nature 436 (7052), 793 (2005)”.

The nucleic acid sequence of 47266012_SOYBEAN from Glycine max, e.g. as shown in the respective line in column 5 of Table I, application no. 4, is unpublished. And the activity of the gene product thereof is the activity of 47266012-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “47266012-protein”, especially from Glycine max or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said 47266012_SOYBEAN, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said 47266012_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said 47266012_SOYBEAN, or a functional equivalent or a homolog thereof as depicted in column 8 of Table II, application no. 4, preferably a homolog or functional equivalent as depicted in column 8 of Table II B, application no. 4, and being depicted in the same respective line as said 47266012_SOYBEAN, and preferably the activity is increased non-targeted

Accordingly, in one embodiment, the molecule which activity is to be increased in the process of the invention is the gene product with an activity as a “47266012-protein”, preferably it is the molecule of section (a) or (b) of this paragraph.

In particular, it was observed that in plants, especially in Arabidopsis thaliana, increasing or generating the activity of a gene product non-targeted with the activity of a “47266012-protein”, preferably being encoded by a gene comprising the nucleic acid sequence SEQ ID NO. 15187, preferably the coding region thereof, conferred the production of or the increase in threonine compared with the wild type control.

The nucleic acid sequence of 49747384_SOYBEAN from Glycine max, e.g. as shown in the respective line in column 5 of Table I, application no. 4, is unpublished. And the activity of the gene product thereof is the activity of 49747384_SOYBEAN-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “49747384_SOYBEAN-protein”, especially from Glycine max or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said 49747384_SOYBEAN, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said 49747384_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said 49747384_SOYBEAN, or a functional equivalent or a homolog thereof as depicted in column 8 of Table II, application no. 4, preferably a homolog or functional equivalent as depicted in column 8 of Table II B, application no. 4, and being depicted in the same respective line as said 49747384_SOYBEAN, and preferably the activity is increased non-targeted.

Accordingly, in one embodiment, the molecule which activity is to be increased in the process of the invention is the gene product with an activity as a “49747384_SOYBEAN-protein”, preferably it is the molecule of section (a) or (b) of this paragraph.

In particular, it was observed that in plants, especially in Arabidopsis thaliana, increasing or generating the activity of a gene product non-targeted with the activity of a “49747384_SOYBEAN-protein”, preferably being encoded by a gene comprising the nucleic acid sequence SEQ ID NO. 69, preferably the coding region thereof, conferred the production of or the increase in threonine compared with the wild type control.

The nucleic acid sequence of 59582753_SOYBEAN from Glycine max, e.g. as shown in the respective line in column 5 of Table I, application no. 4, is unpublished. And the activity of the gene product thereof is the activity of glucose-6-phosphate 1-dehydrogenase.

Accordingly, in one embodiment, the process of the present invention for producing threonine in a non-human organism, like a microorganism or a plant or a part thereof, comprises increasing or generating the activity of a gene product with the activity of a gene product conferring the activity of “glucose-6-phosphate 1-dehydrogenase”, especially from Glycine max or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said 59582753_SOYBEAN, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said 59582753_SOYBEAN, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said 59582753_SOYBEAN, or a functional equivalent or a homolog thereof as depicted in column 8 of Table II, application no. 4, preferably a homolog or functional equivalent as depicted in column 8 of Table II B, application no. 4, and being depicted in the same respective line as said 59582753_SOYBEAN, and preferably the activity is increased plastidic.

Accordingly, in one embodiment, the molecule which activity is to be increased in the process of the invention is the gene product with an activity as a “glucose-6-phosphate 1-dehydrogenase”, preferably it is the molecule of section (a) or (b) of this paragraph.

In particular, it was observed that in plants, especially in Arabidopsis thaliana, increasing or generating the activity of a gene product plastidic with the activity of a “glucose-6-phosphate 1-dehydrogenase”, preferably being encoded by a gene comprising the nucleic acid sequence SEQ ID NO. 16883, preferably the coding region thereof, conferred the production of or the increase in threonine compared with the wild type control.

The nucleic acid sequence of At1g09680 from Arabidopsis thaliana, e.g. as shown in the respective line in column 5 of Table I, application no. 4, has been published in F. And the activity of the gene product thereof is the activity of At1g09680-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “At1g09680-protein”, especially from Arabidopsis thaliana or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said At1g09680, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said At1g09680, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said At1g09680, or a functional equivalent or a homolog thereof as depicted in column 8 of Table II, application no. 4, preferably a homolog or functional equivalent as depicted in column 8 of Table II B, application no. 4, and being depicted in the same respective line as said At1g09680, and preferably the activity is increased non-targeted.

Accordingly, in one embodiment, the molecule which activity is to be increased in the process of the invention is the gene product with an activity as a “At1g09680-protein”, preferably it is the molecule of section (a) or (b) of this paragraph.

In particular, it was observed that in plants, especially in Arabidopsis thaliana, increasing or generating the activity of a gene product non-targeted with the activity of a “At1g09680-protein”, preferably being encoded by a gene comprising the nucleic acid sequence SEQ ID NO. 352, preferably the coding region thereof, conferred the production of or the increase in threonine compared with the wild type control.

The nucleic acid sequence of At1g31230 from Arabidopsis thaliana, e.g. as shown in the respective line in column 5 of Table I, application no. 4, has been published in F. And the activity of the gene product thereof is the activity of bifunctional aspartokinase/homoserine dehydrogenase.

Accordingly, in one embodiment, the process of the present invention for producing threonine in a non-human organism, like a microorganism or a plant or a part thereof, comprises increasing or generating the activity of a gene product with the activity of a gene product conferring the activity of “bifunctional aspartokinase/homoserine dehydrogenase”, especially from Arabidopsis thaliana or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said At1g31230, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said At1g31230, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said At1g31230, or a functional equivalent or a homolog thereof as depicted in column 8 of Table II, application no. 4, preferably a homolog or functional equivalent as depicted in column 8 of Table II B, application no. 4, and being depicted in the same respective line as said At1g31230, and preferably the activity is increased non-targeted.

Accordingly, in one embodiment, the molecule which activity is to be increased in the process of the invention is the gene product with an activity as a “bifunctional aspartokinase/homoserine dehydrogenase”, preferably it is the molecule of section (a) or (b) of this paragraph.

In particular, it was observed that in plants, especially in Arabidopsis thaliana, increasing or generating the activity of a gene product non-targeted with the activity of a “bifunctional aspartokinase/homoserine dehydrogenase”, preferably being encoded by a gene comprising the nucleic acid sequence SEQ ID NO. 627, preferably the coding region thereof, conferred the production of or the increase in threonine compared with the wild type control.

The nucleic acid sequence of At1g36730 from Arabidopsis thaliana, e.g. as shown in the respective line in column 5 of Table I, application no. 4, has been published in F. And the activity of the gene product thereof is the activity of eukaryotic translation initiation factor 5.

Accordingly, in one embodiment, the process of the present invention for producing threonine in a non-human organism, like a microorganism or a plant or a part thereof, comprises increasing or generating the activity of a gene product with the activity of a gene product conferring the activity of “eukaryotic translation initiation factor 5”, especially from Arabidopsis thaliana or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said At1g36730, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said At1g36730, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said At1g36730, or a functional equivalent or a homolog thereof as depicted in column 8 of Table II, application no. 4, preferably a homolog or functional equivalent as depicted in column 8 of Table II B, application no. 4, and being depicted in the same respective line as said At1g36730, and preferably the activity is increased non-targeted.

Accordingly, in one embodiment, the molecule which activity is to be increased in the process of the invention is the gene product with an activity as a “eukaryotic translation initiation factor 5”, preferably it is the molecule of section (a) or (b) of this paragraph.

In particular, it was observed that in plants, especially in Arabidopsis thaliana, increasing or generating the activity of a gene product non-targeted with the activity of a “eukaryotic translation initiation factor 5”, preferably being encoded by a gene comprising the nucleic acid sequence SEQ ID NO. 17968, preferably the coding region thereof, conferred the production of or the increase in threonine compared with the wild type control.

The nucleic acid sequence of At1g43850 from Arabidopsis thaliana, e.g. as shown in the respective line in column 5 of Table I, application no. 4, has been published in F. And the activity of the gene product thereof is the activity of transcriptional regulator.

Accordingly, in one embodiment, the process of the present invention for producing threonine in a non-human organism, like a microorganism or a plant or a part thereof, comprises increasing or generating the activity of a gene product with the activity of a gene product conferring the activity of “transcriptional regulator”, especially from Arabidopsis thaliana or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said At1g43850, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said At1g43850, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said At1g43850, or a functional equivalent or a homolog thereof as depicted in column 8 of Table II, application no. 4, preferably a homolog or functional equivalent as depicted in column 8 of Table II B, application no. 4, and being depicted in the same respective line as said At1g43850, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 18070, preferably the coding region thereof, conferred the production of or the increase in threonine compared with the wild type control.

The nucleic acid sequence of At1g48040 from Arabidopsis thaliana, e.g. as shown in the respective line in column 5 of Table I, application no. 4, has been published in F. And the activity of the gene product thereof is the activity of protein phosphatase.

Accordingly, in one embodiment, the process of the present invention for producing threonine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said At1g48040, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said At1g48040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said At1g48040, or a functional equivalent or a homolog thereof as depicted in column 8 of Table II, application no. 4, preferably a homolog or functional equivalent as depicted in column 8 of Table II B, application no. 4, and being depicted in the same respective line as said At1g48040, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 812, preferably the coding region thereof, conferred the production of or the increase in threonine compared with the wild type control.

The nucleic acid sequence of At1g48260 from Arabidopsis thaliana, e.g. as shown in the respective line in column 5 of Table I, application no. 4, has been published in F. And the activity of the gene product thereof is the activity of protein kinase.

Accordingly, in one embodiment, the process of the present invention for producing threonine in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said At1g48260, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said At1g48260, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said At1g48260, or a functional equivalent or a homolog thereof as depicted in column 8 of Table II, application no. 4, preferably a homolog or functional equivalent as depicted in column 8 of Table II B, application no. 4, and being depicted in the same respective line as said At1g48260, and preferably the activity is increased non-targeted.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 18235, preferably the coding region thereof, conferred the production of or the increase in threonine compared with the wild type control.

The nucleic acid sequence of At1g72770 from Arabidopsis thaliana, e.g. as shown in the respective line in column 5 of Table I, application no. 4, has been published in F. And the activity of the gene product thereof is the activity of protein phosphatase.

Accordingly, in one embodiment, the process of the present invention for producing threonine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said At1g72770, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said At1g72770, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said At1g72770, or a functional equivalent or a homolog thereof as depicted in column 8 of Table II, application no. 4, preferably a homolog or functional equivalent as depicted in column 8 of Table II B, application no. 4, and being depicted in the same respective line as said At1g72770, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19419, preferably the coding region thereof, conferred the production of or the increase in threonine compared with the wild type control.

The nucleic acid sequence of At2g25070 from Arabidopsis thaliana, e.g. as shown in the respective line in column 5 of Table I, application no. 4, has been published in F. And the activity of the gene product thereof is the activity of protein phosphatase.

Accordingly, in one embodiment, the process of the present invention for producing threonine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said At2g25070, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said At2g25070, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said At2g25070, or a functional equivalent or a homolog thereof as depicted in column 8 of Table II, application no. 4, preferably a homolog or functional equivalent as depicted in column 8 of Table II B, application no. 4, and being depicted in the same respective line as said At2g25070, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 1298, preferably the coding region thereof, conferred the production of or the increase in threonine compared with the wild type control.

The nucleic acid sequence of At2g30540 from Arabidopsis thaliana, e.g. as shown in the respective line in column 5 of Table I, application no. 4, has been published in F. And the activity of the gene product thereof is the activity of glutaredoxin.

Accordingly, in one embodiment, the process of the present invention for producing threonine in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said At2g30540, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said At2g30540, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said At2g30540, or a functional equivalent or a homolog thereof as depicted in column 8 of Table II, application no. 4, preferably a homolog or functional equivalent as depicted in column 8 of Table II B, application no. 4, and being depicted in the same respective line as said At2g30540, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 20346, preferably the coding region thereof, conferred the production of or the increase in threonine compared with the wild type control.

The nucleic acid sequence of At3g20910 from Arabidopsis thaliana, e.g. as shown in the respective line in column 5 of Table I, application no. 4, has been published in F. And the activity of the gene product thereof is the activity of CCAAT-binding transcription factor.

Accordingly, in one embodiment, the process of the present invention for producing threonine in a non-human organism, like a microorganism or a plant or a part thereof, comprises increasing or generating the activity of a gene product with the activity of a gene product conferring the activity of “CCAAT-binding transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said At3g20910, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said At3g20910, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said At3g20910, or a functional equivalent or a homolog thereof as depicted in column 8 of Table II, application no. 4, preferably a homolog or functional equivalent as depicted in column 8 of Table II B, application no. 4, and being depicted in the same respective line as said At3g20910, and preferably the activity is increased non-targeted.

Accordingly, in one embodiment, the molecule which activity is to be increased in the process of the invention is the gene product with an activity as a “CCAAT-binding transcription factor”, preferably it is the molecule of section (a) or (b) of this paragraph.

In particular, it was observed that in plants, especially in Arabidopsis thaliana, increasing or generating the activity of a gene product non-targeted with the activity of a “CCAAT-binding transcription factor”, preferably being encoded by a gene comprising the nucleic acid sequence SEQ ID NO. 22921, preferably the coding region thereof, conferred the production of or the increase in threonine compared with the wild type control.

The nucleic acid sequence of At3g23000 from Arabidopsis thaliana, e.g. as shown in the respective line in column 5 of Table I, application no. 4, has been published in F. And the activity of the gene product thereof is the activity of protein kinase.

Accordingly, in one embodiment, the process of the present invention for producing threonine in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said At3g23000, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said At3g23000, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said At3g23000, or a functional equivalent or a homolog thereof as depicted in column 8 of Table II, application no. 4, preferably a homolog or functional equivalent as depicted in column 8 of Table II B, application no. 4, and being depicted in the same respective line as said At3g23000, and preferably the activity is increased non-targeted.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 1815, preferably the coding region thereof, conferred the production of or the increase in threonine compared with the wild type control.

The nucleic acid sequence of At3g27540 from Arabidopsis thaliana, e.g. as shown in the respective line in column 5 of Table I, application no. 4, has been published in F. And the activity of the gene product thereof is the activity of glycosyl transferase.

Accordingly, in one embodiment, the process of the present invention for producing threonine in a non-human organism, like a microorganism or a plant or a part thereof, comprises increasing or generating the activity of a gene product with the activity of a gene product conferring the activity of “glycosyl transferase”, especially from Arabidopsis thaliana or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said At3g27540, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said At3g27540, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said At3g27540, or a functional equivalent or a homolog thereof as depicted in column 8 of Table II, application no. 4, preferably a homolog or functional equivalent as depicted in column 8 of Table II B, application no. 4, and being depicted in the same respective line as said At3g27540, and preferably the activity is increased non-targeted.

Accordingly, in one embodiment, the molecule which activity is to be increased in the process of the invention is the gene product with an activity as a “glycosyl transferase”, preferably it is the molecule of section (a) or (b) of this paragraph.

In particular, it was observed that in plants, especially in Arabidopsis thaliana, increasing or generating the activity of a gene product non-targeted with the activity of a “glycosyl transferase”, preferably being encoded by a gene comprising the nucleic acid sequence SEQ ID NO. 68777, preferably the coding region thereof, conferred the production of or the increase in threonine compared with the wild type control.

The nucleic acid sequence of At3g62930 from Arabidopsis thaliana, e.g. as shown in the respective line in column 5 of Table I, application no. 4, has been published in F. And the activity of the gene product thereof is the activity of monthiol glutaredoxin.

Accordingly, in one embodiment, the process of the present invention for producing threonine in a non-human organism, like a microorganism or a plant or a part thereof, comprises increasing or generating the activity of a gene product with the activity of a gene product conferring the activity of “monthiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said At3g62930, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said At3g62930, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said At3g62930, or a functional equivalent or a homolog thereof as depicted in column 8 of Table II, application no. 4, preferably a homolog or functional equivalent as depicted in column 8 of Table II B, application no. 4, and being depicted in the same respective line as said At3g62930, and preferably the activity is increased non-targeted.

Accordingly, in one embodiment, the molecule which activity is to be increased in the process of the invention is the gene product with an activity as a “monthiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monthiol glutaredoxin”, preferably being encoded by a gene comprising the nucleic acid sequence SEQ ID NO. 2573, preferably the coding region thereof, conferred the production of or the increase in threonine compared with the wild type control.

The nucleic acid sequence of At3g62950 from Arabidopsis thaliana, e.g. as shown in the respective line in column 5 of Table I, application no. 4, has been published in F. And the activity of the gene product thereof is the activity of glutaredoxin.

Accordingly, in one embodiment, the process of the present invention for producing threonine in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said At3g62950, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said At3g62950, or a functional equivalent or a homolog thereof as depicted in column 8 of Table II, application no. 4, preferably a homolog or functional equivalent as depicted in column 8 of Table II B, application no. 4, and being depicted in the same respective line as said At3g62950, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in threonine compared with the wild type control.

The nucleic acid sequence of At4g15660 from Arabidopsis thaliana, e.g. as shown in the respective line in column 5 of Table I, application no. 4, has been published in F. And the activity of the gene product thereof is the activity of monothiol glutaredoxin.

Accordingly, in one embodiment, the process of the present invention for producing threonine in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said At4g15660, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said At4g15660, or a functional equivalent or a homolog thereof as depicted in column 8 of Table II, application no. 4, preferably a homolog or functional equivalent as depicted in column 8 of Table II B, application no. 4, and being depicted in the same respective line as said At4g15660, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in threonine compared with the wild type control.

The nucleic acid sequence of At4g15670 from Arabidopsis thaliana, e.g. as shown in the respective line in column 5 of Table I, application no. 4, has been published in F. And the activity of the gene product thereof is the activity of monothiol glutaredoxin.

Accordingly, in one embodiment, the process of the present invention for producing threonine in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said At4g15670, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said At4g15670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said At4g15670, or a functional equivalent or a homolog thereof as depicted in column 8 of Table II, application no. 4, preferably a homolog or functional equivalent as depicted in column 8 of Table II B, application no. 4, and being depicted in the same respective line as said At4g15670, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3279, preferably the coding region thereof, conferred the production of or the increase in threonine compared with the wild type control.

The nucleic acid sequence of At4g15690 from Arabidopsis thaliana, e.g. as shown in the respective line in column 5 of Table I, application no. 4, has been published in F. And the activity of the gene product thereof is the activity of monothiol glutaredoxin.

Accordingly, in one embodiment, the process of the present invention for producing threonine in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said At4g15690, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said At4g15690, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said At4g15690, or a functional equivalent or a homolog thereof as depicted in column 8 of Table II, application no. 4, preferably a homolog or functional equivalent as depicted in column 8 of Table II B, application no. 4, and being depicted in the same respective line as said At4g15690, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23844, preferably the coding region thereof, conferred the production of or the increase in threonine compared with the wild type control.

The nucleic acid sequence of At4g18880 from Arabidopsis thaliana, e.g. as shown in the respective line in column 5 of Table I, application no. 4, has been published in F. And the activity of the gene product thereof is the activity of heat shock transcription factor.

Accordingly, in one embodiment, the process of the present invention for producing threonine in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said At4g18880, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said At4g18880, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said At4g18880, or a functional equivalent or a homolog thereof as depicted in column 8 of Table II, application no. 4, preferably a homolog or functional equivalent as depicted in column 8 of Table II B, application no. 4, and being depicted in the same respective line as said At4g18880, and preferably the activity is increased non-targeted.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 24232, preferably the coding region thereof, conferred the production of or the increase in threonine compared with the wild type control.

The nucleic acid sequence of At4g32480 from Arabidopsis thaliana, e.g. as shown in the respective line in column 5 of Table I, application no. 4, has been published in F. And the activity of the gene product thereof is the activity of At4g32480-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “At4g32480-protein”, especially from Arabidopsis thaliana or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said At4g32480, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said At4g32480, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said At4g32480, or a functional equivalent or a homolog thereof as depicted in column 8 of Table II, application no. 4, preferably a homolog or functional equivalent as depicted in column 8 of Table II B, application no. 4, and being depicted in the same respective line as said At4g32480, and preferably the activity is increased non-targeted.

Accordingly, in one embodiment, the molecule which activity is to be increased in the process of the invention is the gene product with an activity as a “At4g32480-protein”, preferably it is the molecule of section (a) or (b) of this paragraph.

In particular, it was observed that in plants, especially in Arabidopsis thaliana, increasing or generating the activity of a gene product non-targeted with the activity of a “At4g32480-protein”, preferably being encoded by a gene comprising the nucleic acid sequence SEQ ID NO. 4040, preferably the coding region thereof, conferred the production of or the increase in threonine compared with the wild type control.

The nucleic acid sequence of At4g34160 from Arabidopsis thaliana, e.g. as shown in the respective line in column 5 of Table I, application no. 4, has been published in F. And the activity of the gene product thereof is the activity of cyclin D .

Accordingly, in one embodiment, the process of the present invention for producing threonine in a non-human organism, like a microorganism or a plant or a part thereof, comprises increasing or generating the activity of a gene product with the activity of a gene product conferring the activity of “cyclin D”, especially from Arabidopsis thaliana or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said At4g34160, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said At4g34160, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said At4g34160, or a functional equivalent or a homolog thereof as depicted in column 8 of Table II, application no. 4, preferably a homolog or functional equivalent as depicted in column 8 of Table II B, application no. 4, and being depicted in the same respective line as said At4g34160, and preferably the activity is increased non-targeted.

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 24311, preferably the coding region thereof, conferred the production of or the increase in threonine compared with the wild type control.

The nucleic acid sequence of At4g35310 from Arabidopsis thaliana, e.g. as shown in the respective line in column 5 of Table I, application no. 4, has been published in F. And the activity of the gene product thereof is the activity of calcium-dependent protein kinase.

Accordingly, in one embodiment, the process of the present invention for producing threonine in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said At4g35310, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said At4g35310, or a functional equivalent or a homolog thereof as depicted in column 8 of Table II, application no. 4, preferably a homolog or functional equivalent as depicted in column 8 of Table II B, application no. 4, and being depicted in the same respective line as said At4g35310, and preferably the activity is increased non-targeted.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in threonine compared with the wild type control.

The nucleic acid sequence of At5g07200 from Arabidopsis thaliana, e.g. as shown in the respective line in column 5 of Table I, application no. 4, has been published in F. And the activity of the gene product thereof is the activity of gibberellin 20-oxidase.

Accordingly, in one embodiment, the process of the present invention for producing threonine in a non-human organism, like a microorganism or a plant or a part thereof, comprises increasing or generating the activity of a gene product with the activity of a gene product conferring the activity of “gibberellin 20-oxidase”, especially from Arabidopsis thaliana or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said At5g07200, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said At5g07200, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said At5g07200, or a functional equivalent or a homolog thereof as depicted in column 8 of Table II, application no. 4, preferably a homolog or functional equivalent as depicted in column 8 of Table II B, application no. 4, and being depicted in the same respective line as said At5g07200, and preferably the activity is increased non-targeted.

Accordingly, in one embodiment, the molecule which activity is to be increased in the process of the invention is the gene product with an activity as a “gibberellin 20-oxidase”, preferably it is the molecule of section (a) or (b) of this paragraph.

In particular, it was observed that in plants, especially in Arabidopsis thaliana, increasing or generating the activity of a gene product non-targeted with the activity of a “gibberellin 20-oxidase”, preferably being encoded by a gene comprising the nucleic acid sequence SEQ ID NO. 24492, preferably the coding region thereof, conferred the production of or the increase in threonine compared with the wild type control.

The nucleic acid sequence of At5g13280 from Arabidopsis thaliana, e.g. as shown in the respective line in column 5 of Table I, application no. 4, has been published in F. And the activity of the gene product thereof is the activity of aspartate kinase.

Accordingly, in one embodiment, the process of the present invention for producing threonine in a non-human organism, like a microorganism or a plant or a part thereof, comprises increasing or generating the activity of a gene product with the activity of a gene product conferring the activity of “aspartate kinase”, especially from Arabidopsis thaliana or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said At5g13280, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said At5g13280, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said At5g13280, or a functional equivalent or a homolog thereof as depicted in column 8 of Table II, application no. 4, preferably a homolog or functional equivalent as depicted in column 8 of Table II B, application no. 4, and being depicted in the same respective line as said At5g13280, and preferably the activity is increased non-targeted.

Accordingly, in one embodiment, the molecule which activity is to be increased in the process of the invention is the gene product with an activity as a “aspartate kinase”, preferably it is the molecule of section (a) or (b) of this paragraph.

In particular, it was observed that in plants, especially in Arabidopsis thaliana, increasing or generating the activity of a gene product non-targeted with the activity of a “aspartate kinase”, preferably being encoded by a gene comprising the nucleic acid sequence SEQ ID NO. 93451, preferably the coding region thereof, conferred the production of or the increase in threonine compared with the wild type control.

The nucleic acid sequence of At5g16650 from Arabidopsis thaliana, e.g. as shown in the respective line in column 5 of Table I, application no. 4, has been published in F. And the activity of the gene product thereof is the activity of At5g16650-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “At5g16650-protein”, especially from Arabidopsis thaliana or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said At5g16650, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said At5g16650, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said At5g16650, or a functional equivalent or a homolog thereof as depicted in column 8 of Table II, application no. 4, preferably a homolog or functional equivalent as depicted in column 8 of Table II B, application no. 4, and being depicted in the same respective line as said At5g16650, and preferably the activity is increased non-targeted.

Accordingly, in one embodiment, the molecule which activity is to be increased in the process of the invention is the gene product with an activity as a “At5g16650-protein”, preferably it is the molecule of section (a) or (b) of this paragraph.

In particular, it was observed that in plants, especially in Arabidopsis thaliana, increasing or generating the activity of a gene product non-targeted with the activity of a “At5g16650-protein”, preferably being encoded by a gene comprising the nucleic acid sequence SEQ ID NO. 25283, preferably the coding region thereof, conferred the production of or the increase in threonine compared with the wild type control.

The nucleic acid sequence of At5g39950 from Arabidopsis thaliana, e.g. as shown in the respective line in column 5 of Table I, application no. 4, has been published in F. And the activity of the gene product thereof is the activity of thioredoxin.

Accordingly, in one embodiment, the process of the present invention for producing threonine in a non-human organism, like a microorganism or a plant or a part thereof, comprises increasing or generating the activity of a gene product with the activity of a gene product conferring the activity of “thioredoxin”, especially from Arabidopsis thaliana or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said At5g39950, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said At5g39950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said At5g39950, or a functional equivalent or a homolog thereof as depicted in column 8 of Table II, application no. 4, preferably a homolog or functional equivalent as depicted in column 8 of Table II B, application no. 4, and being depicted in the same respective line as said At5g39950, and preferably the activity is increased non-targeted.

Accordingly, in one embodiment, the molecule which activity is to be increased in the process of the invention is the gene product with an activity as a “thioredoxin”, preferably it is the molecule of section (a) or (b) of this paragraph.

In particular, it was observed that in plants, especially in Arabidopsis thaliana, increasing or generating the activity of a gene product non-targeted with the activity of a “thioredoxin”, preferably being encoded by a gene comprising the nucleic acid sequence SEQ ID NO. 69574, preferably the coding region thereof, conferred the production of or the increase in threonine compared with the wild type control.

The nucleic acid sequence of At5g57050 from Arabidopsis thaliana, e.g. as shown in the respective line in column 5 of Table I, application no. 4, has been published in F. And the activity of the gene product thereof is the activity of protein phosphatase.

Accordingly, in one embodiment, the process of the present invention for producing threonine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said At5g57050, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said At5g57050, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said At5g57050, or a functional equivalent or a homolog thereof as depicted in column 8 of Table II, application no. 4, preferably a homolog or functional equivalent as depicted in column 8 of Table II B, application no. 4, and being depicted in the same respective line as said At5g57050, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 5318, preferably the coding region thereof, conferred the production of or the increase in threonine compared with the wild type control.

The nucleic acid sequence of At5g64920 from Arabidopsis thaliana, e.g. as shown in the respective line in column 5 of Table I, application no. 4, has been published in F. And the activity of the gene product thereof is the activity of zinc finger protein.

Accordingly, in one embodiment, the process of the present invention for producing threonine in a non-human organism, like a microorganism or a plant or a part thereof, comprises increasing or generating the activity of a gene product with the activity of a gene product conferring the activity of “zinc finger protein”, especially from Arabidopsis thaliana or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said At5g64920, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said At5g64920, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said At5g64920, or a functional equivalent or a homolog thereof as depicted in column 8 of Table II, application no. 4, preferably a homolog or functional equivalent as depicted in column 8 of Table II B, application no. 4, and being depicted in the same respective line as said At5g64920, and preferably the activity is increased non-targeted.

Accordingly, in one embodiment, the molecule which activity is to be increased in the process of the invention is the gene product with an activity as a “zinc finger protein”, preferably it is the molecule of section (a) or (b) of this paragraph.

In particular, it was observed that in plants, especially in Arabidopsis thaliana, increasing or generating the activity of a gene product non-targeted with the activity of a “zinc finger protein”, preferably being encoded by a gene comprising the nucleic acid sequence SEQ ID NO. 5493, preferably the coding region thereof, conferred the production of or the increase in threonine compared with the wild type control.

The nucleic acid sequence of AvinDRAFT_{—}0035 from Azotobacter vinelandii, e.g. as shown in the respective line in column 5 of Table I, application no. 4, has been published in F. And the activity of the gene product thereof is the activity of CDP-diacylglycerol-serine O-phosphatidyltransferase.

Accordingly, in one embodiment, the process of the present invention for producing threonine in a non-human organism, like a microorganism or a plant or a part thereof, comprises increasing or generating the activity of a gene product with the activity of a gene product conferring the activity of “CDP-diacylglycerol-serine O-phosphatidyltransferase”, especially from Azotobacter vinelandii or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said AvinDRAFT_{—}0035, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said AvinDRAFT_{—}0035, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said AvinDRAFT_{—}0035, or a functional equivalent or a homolog thereof as depicted in column 8 of Table II, application no. 4, preferably a homolog or functional equivalent as depicted in column 8 of Table II B, application no. 4, and being depicted in the same respective line as said AvinDRAFT_{—}0035, and preferably the activity is increased non-targeted.

Accordingly, in one embodiment, the molecule which activity is to be increased in the process of the invention is the gene product with an activity as a “CDP-diacylglycerol-serine O-phosphatidyltransferase”, preferably it is the molecule of section (a) or (b) of this paragraph.

In particular, it was observed that in plants, especially in Arabidopsis thaliana, increasing or generating the activity of a gene product non-targeted with the activity of a “CDP-diacylglycerolserine O-phosphatidyltransferase”, preferably being encoded by a gene comprising the nucleic acid sequence SEQ ID NO. 70583, preferably the coding region thereof, conferred the production of or the increase in threonine compared with the wild type control.

The nucleic acid sequence of AvinDRAFT_{—}1495 from Azotobacter vinelandii, e.g. as shown in the respective line in column 5 of Table I, application no. 4, has been published in F. And the activity of the gene product thereof is the activity of malic enzyme.

Accordingly, in one embodiment, the process of the present invention for producing threonine in a non-human organism, like a microorganism or a plant or a part thereof, comprises increasing or generating the activity of a gene product with the activity of a gene product conferring the activity of “malic enzyme”, especially from Azotobacter vinelandii or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said AvinDRAFT_{—}1495, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said AvinDRAFT_{—}1495, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said AvinDRAFT_{—}1495, or a functional equivalent or a homolog thereof as depicted in column 8 of Table II, application no. 4, preferably a homolog or functional equivalent as depicted in column 8 of Table II B, application no. 4, and being depicted in the same respective line as said AvinDRAFT_{—}1495, and preferably the activity is increased non-targeted.

Accordingly, in one embodiment, the molecule which activity is to be increased in the process of the invention is the gene product with an activity as a “malic enzyme”, preferably it is the molecule of section (a) or (b) of this paragraph.

In particular, it was observed that in plants, especially in Arabidopsis thaliana, increasing or generating the activity of a gene product non-targeted with the activity of a “malic enzyme”, preferably being encoded by a gene comprising the nucleic acid sequence SEQ ID NO. 5557, preferably the coding region thereof, conferred the production of or the increase in threonine compared with the wild type control.

The nucleic acid sequence of AvinDRAFT_{—}1624 from Azotobacter vinelandii, e.g. as shown in the respective line in column 5 of Table I, application no. 4, has been published in F. And the activity of the gene product thereof is the activity of sec-independent protein translocase.

Accordingly, in one embodiment, the process of the present invention for producing threonine in a non-human organism, like a microorganism or a plant or a part thereof, comprises increasing or generating the activity of a gene product with the activity of a gene product conferring the activity of “sec-independent protein translocase”, especially from Azotobacter vinelandii or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said AvinDRAFT_{—}1624, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said AvinDRAFT_{—}1624, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said AvinDRAFT_{—}1624, or a functional equivalent or a homolog thereof as depicted in column 8 of Table II, application no. 4, preferably a homolog or functional equivalent as depicted in column 8 of Table II B, application no. 4, and being depicted in the same respective line as said AvinDRAFT_{—}1624, and preferably the activity is increased non-targeted.

Accordingly, in one embodiment, the molecule which activity is to be increased in the process of the invention is the gene product with an activity as a “sec-independent protein translocase”, preferably it is the molecule of section (a) or (b) of this paragraph.

In particular, it was observed that in plants, especially in Arabidopsis thaliana, increasing or generating the activity of a gene product non-targeted with the activity of a “sec-independent protein translocase”, preferably being encoded by a gene comprising the nucleic acid sequence SEQ ID NO. 26196, preferably the coding region thereof, conferred the production of or the increase in threonine compared with the wild type control.

The nucleic acid sequence of AvinDRAFT_{—}2091 from Azotobacter vinelandii, e.g. as shown in the respective line in column 5 of Table I, application no. 4, has been published in F. And the activity of the gene product thereof is the activity of beta-hydroxylase.

Accordingly, in one embodiment, the process of the present invention for producing threonine in a non-human organism, like a microorganism or a plant or a part thereof, comprises increasing or generating the activity of a gene product with the activity of a gene product conferring the activity of “beta-hydroxylase”, especially from Azotobacter vinelandii or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said AvinDRAFT_{—}2091, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said AvinDRAFT_{—}2091, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said AvinDRAFT_{—}2091, or a functional equivalent or a homolog thereof as depicted in column 8 of Table II, application no. 4, preferably a homolog or functional equivalent as depicted in column 8 of Table II B, application no. 4, and being depicted in the same respective line as said AvinDRAFT_{—}2091, and preferably the activity is increased non-targeted.

Accordingly, in one embodiment, the molecule which activity is to be increased in the process of the invention is the gene product with an activity as a “beta-hydroxylase”, preferably it is the molecule of section (a) or (b) of this paragraph.

In particular, it was observed that in plants, especially in Arabidopsis thaliana, increasing or generating the activity of a gene product non-targeted with the activity of a “beta-hydroxylase”, preferably being encoded by a gene comprising the nucleic acid sequence SEQ ID NO. 6040, preferably the coding region thereof, conferred the production of or the increase in threonine compared with the wild type control.

The nucleic acid sequence of AvinDRAFT_{—}2344 from Azotobacter vinelandii, e.g. as shown in the respective line in column 5 of Table I, application no. 4, has been published in F. And the activity of the gene product thereof is the activity of elongation factor Tu.

Accordingly, in one embodiment, the process of the present invention for producing threonine in a non-human organism, like a microorganism or a plant or a part thereof, comprises increasing or generating the activity of a gene product with the activity of a gene product conferring the activity of “elongation factor Tu”, especially from Azotobacter vinelandii or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said AvinDRAFT_{—}2344, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said AvinDRAFT_{—}2344, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said AvinDRAFT_{—}2344, or a functional equivalent or a homolog thereof as depicted in column 8 of Table II, application no. 4, preferably a homolog or functional equivalent as depicted in column 8 of Table II B, application no. 4, and being depicted in the same respective line as said AvinDRAFT_{—}2344, and preferably the activity is increased non-targeted.

Accordingly, in one embodiment, the molecule which activity is to be increased in the process of the invention is the gene product with an activity as a “elongation factor Tu”, preferably it is the molecule of section (a) or (b) of this paragraph.

In particular, it was observed that in plants, especially in Arabidopsis thaliana, increasing or generating the activity of a gene product non-targeted with the activity of a “elongation factor Tu”, preferably being encoded by a gene comprising the nucleic acid sequence SEQ ID NO. 27021, preferably the coding region thereof, conferred the production of or the increase in threonine compared with the wild type control.

The nucleic acid sequence of AvinDRAFT_{—}2521 from Azotobacter vinelandii, e.g. as shown in the respective line in column 5 of Table I, application no. 4, has been published in F. And the activity of the gene product thereof is the activity of ABC transporter permease protein.

Accordingly, in one embodiment, the process of the present invention for producing threonine in a non-human organism, like a microorganism or a plant or a part thereof, comprises increasing or generating the activity of a gene product with the activity of a gene product conferring the activity of “ABC transporter permease protein”, especially from Azotobacter vinelandii or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said AvinDRAFT_{—}2521, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said AvinDRAFT_{—}2521, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said AvinDRAFT_{—}2521, or a functional equivalent or a homolog thereof as depicted in column 8 of Table II, application no. 4, preferably a homolog or functional equivalent as depicted in column 8 of Table II B, application no. 4, and being depicted in the same respective line as said AvinDRAFT_{—}2521, and preferably the activity is increased non-targeted.

Accordingly, in one embodiment, the molecule which activity is to be increased in the process of the invention is the gene product with an activity as a “ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “ABC transporter permease protein”, preferably being encoded by a gene comprising the nucleic acid sequence SEQ ID NO. 27882, preferably the coding region thereof, conferred the production of or the increase in threonine compared with the wild type control.

The nucleic acid sequence of AvinDRAFT_{—}2754 from Azotobacter vinelandii, e.g. as shown in the respective line in column 5 of Table I, application no. 4, has been published in F. And the activity of the gene product thereof is the activity of acyl-CoA synthase.

Accordingly, in one embodiment, the process of the present invention for producing threonine in a non-human organism, like a microorganism or a plant or a part thereof, comprises increasing or generating the activity of a gene product with the activity of a gene product conferring the activity of “acyl-CoA synthase”, especially from Azotobacter vinelandii or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said AvinDRAFT_{—}2754, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said AvinDRAFT_{—}2754, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said AvinDRAFT_{—}2754, or a functional equivalent or a homolog thereof as depicted in column 8 of Table II, application no. 4, preferably a homolog or functional equivalent as depicted in column 8 of Table II B, application no. 4, and being depicted in the same respective line as said AvinDRAFT_{—}2754, and preferably the activity is increased non-targeted.

Accordingly, in one embodiment, the molecule which activity is to be increased in the process of the invention is the gene product with an activity as a “acyl-CoA synthase”, preferably it is the molecule of section (a) or (b) of this paragraph.

In particular, it was observed that in plants, especially in Arabidopsis thaliana, increasing or generating the activity of a gene product non-targeted with the activity of a “acyl-CoA synthase”, preferably being encoded by a gene comprising the nucleic acid sequence SEQ ID NO. 28040, preferably the coding region thereof, conferred the production of or the increase in threonine compared with the wild type control.

The nucleic acid sequence of AvinDRAFT_{—}3028 from Azotobacter vinelandii, e.g. as shown in the respective line in column 5 of Table I, application no. 4, has been published in F. And the activity of the gene product thereof is the activity of 2-oxoglutarate dehydrogenase E1 subunit.

Accordingly, in one embodiment, the process of the present invention for producing threonine in a non-human organism, like a microorganism or a plant or a part thereof, comprises increasing or generating the activity of a gene product with the activity of a gene product conferring the activity of “2-oxoglutarate dehydrogenase E1 subunit”, especially from Azotobacter vinelandii or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said AvinDRAFT_{—}3028, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said AvinDRAFT_{—}3028, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said AvinDRAFT_{—}3028, or a functional equivalent or a homolog thereof as depicted in column 8 of Table II, application no. 4, preferably a homolog or functional equivalent as depicted in column 8 of Table II B, application no. 4, and being depicted in the same respective line as said AvinDRAFT_{—}3028, and preferably the activity is increased non-targeted.

Accordingly, in one embodiment, the molecule which activity is to be increased in the process of the invention is the gene product with an activity as a “2-oxoglutarate dehydrogenase E1 subunit”, preferably it is the molecule of section (a) or (b) of this paragraph.

In particular, it was observed that in plants, especially in Arabidopsis thaliana, increasing or generating the activity of a gene product non-targeted with the activity of a “2-oxoglutarate dehydrogenase E1 subunit”, preferably being encoded by a gene comprising the nucleic acid sequence SEQ ID NO. 6075, preferably the coding region thereof, conferred the production of or the increase in threonine compared with the wild type control.

The nucleic acid sequence of AvinDRAFT_{—}3086 from Azotobacter vinelandii, e.g. as shown in the respective line in column 5 of Table I, application no. 4, has been published in F. And the activity of the gene product thereof is the activity of permease protein of peptide ABC transporter.

Accordingly, in one embodiment, the process of the present invention for producing threonine in a non-human organism, like a microorganism or a plant or a part thereof, comprises increasing or generating the activity of a gene product with the activity of a gene product conferring the activity of “permease protein of peptide ABC transporter”, especially from Arabidopsis thaliana or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said AvinDRAFT_{—}3086, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said AvinDRAFT_{—}3086, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said AvinDRAFT_{—}3086, or a functional equivalent or a homolog thereof as depicted in column 8 of Table II, application no. 4, preferably a homolog or functional equivalent as depicted in column 8 of Table II B, application no. 4, and being depicted in the same respective line as said AvinDRAFT_{—}3086, and preferably the activity is increased non-targeted.

Accordingly, in one embodiment, the molecule which activity is to be increased in the process of the invention is the gene product with an activity as a “permease protein of peptide ABC transporter”, preferably it is the molecule of section (a) or (b) of this paragraph.

In particular, it was observed that in plants, especially in Arabidopsis thaliana, increasing or generating the activity of a gene product non-targeted with the activity of a “permease protein of peptide ABC transporter”, preferably being encoded by a gene comprising the nucleic acid sequence SEQ ID NO. 93532, preferably the coding region thereof, conferred the production of or the increase in threonine compared with the wild type control.

The nucleic acid sequence of AvinDRAFT_{—}3135 from Azotobacter vinelandii, e.g. as shown in the respective line in column 5 of Table I, application no. 4, has been published in F. And the activity of the gene product thereof is the activity of GDP-mannose dehydrogenase.

Accordingly, in one embodiment, the process of the present invention for producing threonine in a non-human organism, like a microorganism or a plant or a part thereof, comprises increasing or generating the activity of a gene product with the activity of a gene product conferring the activity of “GDP-mannose dehydrogenase”, especially from Azotobacter vinelandii or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said AvinDRAFT_{—}3135, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said AvinDRAFT_{—}3135, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said AvinDRAFT_{—}3135, or a functional equivalent or a homolog thereof as depicted in column 8 of Table II, application no. 4, preferably a homolog or functional equivalent as depicted in column 8 of Table II B, application no. 4, and being depicted in the same respective line as said AvinDRAFT_{—}3135, and preferably the activity is increased non-targeted.

Accordingly, in one embodiment, the molecule which activity is to be increased in the process of the invention is the gene product with an activity as a “GDP-mannose dehydrogenase”, preferably it is the molecule of section (a) or (b) of this paragraph.

In particular, it was observed that in plants, especially in Arabidopsis thaliana, increasing or generating the activity of a gene product non-targeted with the activity of a “GDP-mannose dehydrogenase”, preferably being encoded by a gene comprising the nucleic acid sequence SEQ ID NO. 72346, preferably the coding region thereof, conferred the production of or the increase in threonine compared with the wild type control.

The nucleic acid sequence of AvinDRAFT_{—}3159 from Azotobacter vinelandii, e.g. as shown in the respective line in column 5 of Table I, application no. 4, has been published in F. And the activity of the gene product thereof is the activity of enoyl-CoA hydratase.

Accordingly, in one embodiment, the process of the present invention for producing threonine in a non-human organism, like a microorganism or a plant or a part thereof, comprises increasing or generating the activity of a gene product with the activity of a gene product conferring the activity of “enoyl-CoA hydratase”, especially from Azotobacter vinelandii or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said AvinDRAFT_{—}3159, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said AvinDRAFT_{—}3159, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said AvinDRAFT_{—}3159, or a functional equivalent or a homolog thereof as depicted in column 8 of Table II, application no. 4, preferably a homolog or functional equivalent as depicted in column 8 of Table II B, application no. 4, and being depicted in the same respective line as said AvinDRAFT_{—}3159, and preferably the activity is increased non-targeted.

Accordingly, in one embodiment, the molecule which activity is to be increased in the process of the invention is the gene product with an activity as a “enoyl-CoA hydratase”, preferably it is the molecule of section (a) or (b) of this paragraph.

In particular, it was observed that in plants, especially in Arabidopsis thaliana, increasing or generating the activity of a gene product non-targeted with the activity of a “enoyl-CoA hydratase”, preferably being encoded by a gene comprising the nucleic acid sequence SEQ ID NO. 28738, preferably the coding region thereof, conferred the production of or the increase in threonine compared with the wild type control.

The nucleic acid sequence of AvinDRAFT_{—}3209 from Azotobacter vinelandii, e.g. as shown in the respective line in column 5 of Table I, application no. 4, has been published in F. And the activity of the gene product thereof is the activity of transcriptional regulator.

Accordingly, in one embodiment, the process of the present invention for producing threonine in a non-human organism, like a microorganism or a plant or a part thereof, comprises increasing or generating the activity of a gene product with the activity of a gene product conferring the activity of “transcriptional regulator”, especially from Azotobacter vinelandii or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said AvinDRAFT_{—}3209, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said AvinDRAFT_{—}3209, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said AvinDRAFT_{—}3209, or a functional equivalent or a homolog thereof as depicted in column 8 of Table II, application no. 4, preferably a homolog or functional equivalent as depicted in column 8 of Table II B, application no. 4, and being depicted in the same respective line as said AvinDRAFT_{—}3209, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 29286, preferably the coding region thereof, conferred the production of or the increase in threonine compared with the wild type control.

The nucleic acid sequence of AvinDRAFT_{—}3546 from Azotobacter vinelandii, e.g. as shown in the respective line in column 5 of Table I, application no. 4, has been published in F. And the activity of the gene product thereof is the activity of homoserine dehydrogenase.

Accordingly, in one embodiment, the process of the present invention for producing threonine in a non-human organism, like a microorganism or a plant or a part thereof, comprises increasing or generating the activity of a gene product with the activity of a gene product conferring the activity of “homoserine dehydrogenase”, especially from Azotobacter vinelandii or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said AvinDRAFT_{—}3546, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said AvinDRAFT_{—}3546, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said AvinDRAFT_{—}3546, or a functional equivalent or a homolog thereof as depicted in column 8 of Table II, application no. 4, preferably a homolog or functional equivalent as depicted in column 8 of Table II B, application no. 4, and being depicted in the same respective line as said AvinDRAFT_{—}3546, and preferably the activity is increased non-targeted.

Accordingly, in one embodiment, the molecule which activity is to be increased in the process of the invention is the gene product with an activity as a “homoserine dehydrogenase”, preferably it is the molecule of section (a) or (b) of this paragraph.

In particular, it was observed that in plants, especially in Arabidopsis thaliana, increasing or generating the activity of a gene product non-targeted with the activity of a “homoserine dehydrogenase”, preferably being encoded by a gene comprising the nucleic acid sequence SEQ ID NO. 6268, preferably the coding region thereof, conferred the production of or the increase in threonine compared with the wild type control.

The nucleic acid sequence of AvinDRAFT_{—}5103 from Azotobacter vinelandii, e.g. as shown in the respective line in column 5 of Table I, application no. 4, has been published in F. And the activity of the gene product thereof is the activity of hydrolase.

Accordingly, in one embodiment, the process of the present invention for producing threonine in a non-human organism, like a microorganism or a plant or a part thereof, comprises increasing or generating the activity of a gene product with the activity of a gene product conferring the activity of “hydrolase”, especially from Azotobacter vinelandii or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said AvinDRAFT_{—}5103, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said AvinDRAFT_{—}5103, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said AvinDRAFT_{—}5103, or a functional equivalent or a homolog thereof as depicted in column 8 of Table II, application no. 4, preferably a homolog or functional equivalent as depicted in column 8 of Table II B, application no. 4, and being depicted in the same respective line as said AvinDRAFT_{—}5103, and preferably the activity is increased non-targeted.

Accordingly, in one embodiment, the molecule which activity is to be increased in the process of the invention is the gene product with an activity as a “hydrolase”, preferably it is the molecule of section (a) or (b) of this paragraph.

In particular, it was observed that in plants, especially in Arabidopsis thaliana, increasing or generating the activity of a gene product non-targeted with the activity of a “hydrolase”, preferably being encoded by a gene comprising the nucleic acid sequence SEQ ID NO. 6510, preferably the coding region thereof, conferred the production of or the increase in threonine compared with the wild type control.

The nucleic acid sequence of AvinDRAFT_{—}5246 from Azotobacter vinelandii, e.g. as shown in the respective line in column 5 of Table I, application no. 4, has been published in F. And the activity of the gene product thereof is the activity of glutamate-ammonia-ligase.

Accordingly, in one embodiment, the process of the present invention for producing threonine in a non-human organism, like a microorganism or a plant or a part thereof, comprises increasing or generating the activity of a gene product with the activity of a gene product conferring the activity of “glutamate-ammonia-ligase”, especially from Azotobacter vinelandii or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said AvinDRAFT_{—}5246, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said AvinDRAFT_{—}5246, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said AvinDRAFT_{—}5246, or a functional equivalent or a homolog thereof as depicted in column 8 of Table II, application no. 4, preferably a homolog or functional equivalent as depicted in column 8 of Table II B, application no. 4, and being depicted in the same respective line as said AvinDRAFT_{—}5246, and preferably the activity is increased non-targeted.

Accordingly, in one embodiment, the molecule which activity is to be increased in the process of the invention is the gene product with an activity as a “glutamate-ammonia-ligase”, preferably it is the molecule of section (a) or (b) of this paragraph.

In particular, it was observed that in plants, especially in Arabidopsis thaliana, increasing or generating the activity of a gene product non-targeted with the activity of a “glutamate-ammonia-ligase”, preferably being encoded by a gene comprising the nucleic acid sequence SEQ ID NO. 32037, preferably the coding region thereof, conferred the production of or the increase in threonine compared with the wild type control.

The nucleic acid sequence of AvinDRAFT_{—}5579 from Azotobacter vinelandii, e.g. as shown in the respective line in column 5 of Table I, application no. 4, has been published in F. And the activity of the gene product thereof is the activity of peptidase.

Accordingly, in one embodiment, the process of the present invention for producing threonine in a non-human organism, like a microorganism or a plant or a part thereof, comprises increasing or generating the activity of a gene product with the activity of a gene product conferring the activity of “peptidase”, especially from Azotobacter vinelandii or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said AvinDRAFT_{—}5579, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said AvinDRAFT_{—}5579, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said AvinDRAFT_{—}5579, or a functional equivalent or a homolog thereof as depicted in column 8 of Table II, application no. 4, preferably a homolog or functional equivalent as depicted in column 8 of Table II B, application no. 4, and being depicted in the same respective line as said AvinDRAFT_{—}5579, and preferably the activity is increased non-targeted.

Accordingly, in one embodiment, the molecule which activity is to be increased in the process of the invention is the gene product with an activity as a “peptidase”, preferably it is the molecule of section (a) or (b) of this paragraph.

In particular, it was observed that in plants, especially in Arabidopsis thaliana, increasing or generating the activity of a gene product non-targeted with the activity of a “peptidase”, preferably being encoded by a gene comprising the nucleic acid sequence SEQ ID NO. 74662, preferably the coding region thereof, conferred the production of or the increase in threonine compared with the wild type control.

The nucleic acid sequence of AvinDRAFT_{—}6093 from Azotobacter vinelandii, e.g. as shown in the respective line in column 5 of Table I, application no. 4, has been published in F. And the activity of the gene product thereof is the activity of Chaperone protein CIpB.

Accordingly, in one embodiment, the process of the present invention for producing threonine in a non-human organism, like a microorganism or a plant or a part thereof, comprises increasing or generating the activity of a gene product with the activity of a gene product conferring the activity of “Chaperone protein CIpB”, especially from Azotobacter vinelandii or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said AvinDRAFT_{—}6093, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said AvinDRAFT_{—}6093, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said AvinDRAFT_{—}6093, or a functional equivalent or a homolog thereof as depicted in column 8 of Table II, application no. 4, preferably a homolog or functional equivalent as depicted in column 8 of Table II B, application no. 4, and being depicted in the same respective line as said AvinDRAFT_{—}6093, and preferably the activity is increased non-targeted.

Accordingly, in one embodiment, the molecule which activity is to be increased in the process of the invention is the gene product with an activity as a “Chaperone protein CIpB”, preferably it is the molecule of section (a) or (b) of this paragraph.

In particular, it was observed that in plants, especially in Arabidopsis thaliana, increasing or generating the activity of a gene product non-targeted with the activity of a “Chaperone protein CIpB”, preferably being encoded by a gene comprising the nucleic acid sequence SEQ ID NO. 33596, preferably the coding region thereof, conferred the production of or the increase in threonine compared with the wild type control.

The nucleic acid sequence of AvinDRAFT_{—}6864 from Azotobacter vinelandii, e.g. as shown in the respective line in column 5 of Table I, application no. 4, has been published in F. And the activity of the gene product thereof is the activity of transcriptional regulator protein.

Accordingly, in one embodiment, the process of the present invention for producing threonine in a non-human organism, like a microorganism or a plant or a part thereof, comprises increasing or generating the activity of a gene product with the activity of a gene product conferring the activity of “transcriptional regulator protein”, especially from Azotobacter vinelandii or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said AvinDRAFT_{—}6864, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said AvinDRAFT_{—}6864, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said AvinDRAFT_{—}6864, or a functional equivalent or a homolog thereof as depicted in column 8 of Table II, application no. 4, preferably a homolog or functional equivalent as depicted in column 8 of Table II B, application no. 4, and being depicted in the same respective line as said AvinDRAFT_{—}6864, and preferably the activity is increased non-targeted.

Accordingly, in one embodiment, the molecule which activity is to be increased in the process of the invention is the gene product with an activity as a “transcriptional regulator protein”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator protein”, preferably being encoded by a gene comprising the nucleic acid sequence SEQ ID NO. 34204, preferably the coding region thereof, conferred the production of or the increase in threonine compared with the wild type control.

The nucleic acid sequence of AX653549 from Oryza sativa, e.g. as shown in the respective line in column 5 of Table I, application no. 4, has been published in F. And the activity of the gene product thereof is the activity of AX653549-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “AX653549-protein”, especially from Oryza sativa or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said AX653549, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said AX653549, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said AX653549, or a functional equivalent or a homolog thereof as depicted in column 8 of Table II, application no. 4, preferably a homolog or functional equivalent as depicted in column 8 of Table II B, application no. 4, and being depicted in the same respective line as said AX653549, and preferably the activity is increased non-targeted.

Accordingly, in one embodiment, the molecule which activity is to be increased in the process of the invention is the gene product with an activity as a “AX653549-protein”, preferably it is the molecule of section (a) or (b) of this paragraph.

In particular, it was observed that in plants, especially in Arabidopsis thaliana, increasing or generating the activity of a gene product non-targeted with the activity of a “AX653549-protein”, preferably being encoded by a gene comprising the nucleic acid sequence SEQ ID NO. 34301, preferably the coding region thereof, conferred the production of or the increase in threonine compared with the wild type control.

The nucleic acid sequence of B0004 from Escherichia coli, e.g. as shown in the respective line in column 5 of Table I, application no. 4, has been published in B. And the activity of the gene product thereof is the activity of threonine synthase.

Accordingly, in one embodiment, the process of the present invention for producing threonine in a non-human organism, like a microorganism or a plant or a part thereof, comprises increasing or generating the activity of a gene product with the activity of a gene product conferring the activity of “threonine synthase”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B0004, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B0004, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B0004, or a functional equivalent or a homolog thereof as depicted in column 8 of Table II, application no. 4, preferably a homolog or functional equivalent as depicted in column 8 of Table II B, application no. 4, and being depicted in the same respective line as said B0004, and preferably the activity is increased plastidic.

Accordingly, in one embodiment, the molecule which activity is to be increased in the process of the invention is the gene product with an activity as a “threonine synthase”, preferably it is the molecule of section (a) or (b) of this paragraph.

In particular, it was observed that in plants, especially in Arabidopsis thaliana, increasing or generating the activity of a gene product plastidic with the activity of a “threonine synthase”, preferably being encoded by a gene comprising the nucleic acid sequence SEQ ID NO. 34889, preferably the coding region thereof, conferred the production of or the increase in threonine compared with the wild type control.

The nucleic acid sequence of B0078 from Escherichia coli, e.g. as shown in the respective line in column 5 of Table I, application no. 4, has been published in B. And the activity of the gene product thereof is the activity of acetolactate synthase small subunit.

Accordingly, in one embodiment, the process of the present invention for producing threonine in a non-human organism, like a microorganism or a plant or a part thereof, comprises increasing or generating the activity of a gene product with the activity of a gene product conferring the activity of “acetolactate synthase small subunit”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B0078, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B0078, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B0078, or a functional equivalent or a homolog thereof as depicted in column 8 of Table II, application no. 4, preferably a homolog or functional equivalent as depicted in column 8 of Table II B, application no. 4, and being depicted in the same respective line as said B0078, and preferably the activity is increased non-targeted.

Accordingly, in one embodiment, the molecule which activity is to be increased in the process of the invention is the gene product with an activity as a “acetolactate synthase small subunit”, preferably it is the molecule of section (a) or (b) of this paragraph.

In particular, it was observed that in plants, especially in Arabidopsis thaliana, increasing or generating the activity of a gene product non-targeted with the activity of a “acetolactate synthase small subunit”, preferably being encoded by a gene comprising the nucleic acid sequence SEQ ID NO. 6818, preferably the coding region thereof, conferred the production of or the increase in threonine compared with the wild type control.

The nucleic acid sequence of B0115 from Escherichia coli, e.g. as shown in the respective line in column 5 of Table I, application no. 4, has been published in B. And the activity of the gene product thereof is the activity of dihydrolipoamide acetyltransferase.

Accordingly, in one embodiment, the process of the present invention for producing threonine in a non-human organism, like a microorganism or a plant or a part thereof, comprises increasing or generating the activity of a gene product with the activity of a gene product conferring the activity of “dihydrolipoamide acetyltransferase”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B0115, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B0115, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B0115, or a functional equivalent or a homolog thereof as depicted in column 8 of Table II, application no. 4, preferably a homolog or functional equivalent as depicted in column 8 of Table II B, application no. 4, and being depicted in the same respective line as said B0115, and preferably the activity is increased plastidic.

Accordingly, in one embodiment, the molecule which activity is to be increased in the process of the invention is the gene product with an activity as a “dihydrolipoamide acetyltransferase”, preferably it is the molecule of section (a) or (b) of this paragraph.

In particular, it was observed that in plants, especially in Arabidopsis thaliana, increasing or generating the activity of a gene product plastidic with the activity of a “dihydrolipoamide acetyltransferase”, preferably being encoded by a gene comprising the nucleic acid sequence SEQ ID NO. 35366, preferably the coding region thereof, conferred the production of or the increase in threonine compared with the wild type control.

The nucleic acid sequence of B0124 from Escherichia coli, e.g. as shown in the respective line in column 5 of Table I, application no. 4, has been published in B. And the activity of the gene product thereof is the activity of glucose dehydrogenase.

Accordingly, in one embodiment, the process of the present invention for producing threonine in a non-human organism, like a microorganism or a plant or a part thereof, comprises increasing or generating the activity of a gene product with the activity of a gene product conferring the activity of “glucose dehydrogenase”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B0124, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B0124, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B0124, or a functional equivalent or a homolog thereof as depicted in column 8 of Table II, application no. 4, preferably a homolog or functional equivalent as depicted in column 8 of Table II B, application no. 4, and being depicted in the same respective line as said B0124, and preferably the activity is increased non-targeted.

Accordingly, in one embodiment, the molecule which activity is to be increased in the process of the invention is the gene product with an activity as a “glucose dehydrogenase”, preferably it is the molecule of section (a) or (b) of this paragraph.

In particular, it was observed that in plants, especially in Arabidopsis thaliana, increasing or generating the activity of a gene product non-targeted with the activity of a “glucose dehydrogenase”, preferably being encoded by a gene comprising the nucleic acid sequence SEQ ID NO. 35482, preferably the coding region thereof, conferred the production of or the increase in threonine compared with the wild type control.

The nucleic acid sequence of B0161 from Escherichia coli, e.g. as shown in the respective line in column 5 of Table I, application no. 4, has been published in B. And the activity of the gene product thereof is the activity of serine protease.

Accordingly, in one embodiment, the process of the present invention for producing threonine in a non-human organism, like a microorganism or a plant or a part thereof, comprises increasing or generating the activity of a gene product with the activity of a gene product conferring the activity of “serine protease”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B0161, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B0161, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B0161, or a functional equivalent or a homolog thereof as depicted in column 8 of Table II, application no. 4, preferably a homolog or functional equivalent as depicted in column 8 of Table II B, application no. 4, and being depicted in the same respective line as said B0161, and preferably the activity is increased non-targeted.

Accordingly, in one embodiment, the molecule which activity is to be increased in the process of the invention is the gene product with an activity as a “serine protease”, preferably it is the molecule of section (a) or (b) of this paragraph.

In particular, it was observed that in plants, especially in Arabidopsis thaliana, increasing or generating the activity of a gene product non-targeted with the activity of a “serine protease”, preferably being encoded by a gene comprising the nucleic acid sequence SEQ ID NO. 7081, preferably the coding region thereof, conferred the production of or the increase in threonine compared with the wild type control.

The nucleic acid sequence of B0221 from Escherichia coli, e.g. as shown in the respective line in column 5 of Table I, application no. 4, has been published in B. And the activity of the gene product thereof is the activity of acyl-CoA dehydrogenase.

Accordingly, in one embodiment, the process of the present invention for producing threonine in a non-human organism, like a microorganism or a plant or a part thereof, comprises increasing or generating the activity of a gene product with the activity of a gene product conferring the activity of “acyl-CoA dehydrogenase”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B0221, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B0221, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B0221, or a functional equivalent or a homolog thereof as depicted in column 8 of Table II, application no. 4, preferably a homolog or functional equivalent as depicted in column 8 of Table II B, application no. 4, and being depicted in the same respective line as said B0221, and preferably the activity is increased non-targeted.

Accordingly, in one embodiment, the molecule which activity is to be increased in the process of the invention is the gene product with an activity as a “acyl-CoA dehydrogenase”, preferably it is the molecule of section (a) or (b) of this paragraph.

In particular, it was observed that in plants, especially in Arabidopsis thaliana, increasing or generating the activity of a gene product non-targeted with the activity of a “acyl-CoA dehydrogenase”, preferably being encoded by a gene comprising the nucleic acid sequence SEQ ID NO. 35590, preferably the coding region thereof, conferred the production of or the increase in threonine compared with the wild type control.

The nucleic acid sequence of B0344 from Escherichia coli, e.g. as shown in the respective line in column 5 of Table I, application no. 4, has been published in B. And the activity of the gene product thereof is the activity of beta-galactosidase.

Accordingly, in one embodiment, the process of the present invention for producing threonine in a non-human organism, like a microorganism or a plant or a part thereof, comprises increasing or generating the activity of a gene product with the activity of a gene product conferring the activity of “beta-galactosidase”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B0344, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B0344, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B0344, or a functional equivalent or a homolog thereof as depicted in column 8 of Table II, application no. 4, preferably a homolog or functional equivalent as depicted in column 8 of Table II B, application no. 4, and being depicted in the same respective line as said B0344, and preferably the activity is increased plastidic.

Accordingly, in one embodiment, the molecule which activity is to be increased in the process of the invention is the gene product with an activity as a “beta-galactosidase”, preferably it is the molecule of section (a) or (b) of this paragraph.

In particular, it was observed that in plants, especially in Arabidopsis thaliana, increasing or generating the activity of a gene product plastidic with the activity of a “beta-galactosidase”, preferably being encoded by a gene comprising the nucleic acid sequence SEQ ID NO. 35733, preferably the coding region thereof, conferred the production of or the increase in threonine compared with the wild type control. The nucleic acid sequence of B0348 from Escherichia coli, e.g. as shown in the respective line in column 5 of Table I, application no. 4, has been published in B. And the activity of the gene product thereof is the activity of 2,3-dihydroxyphenylpropionate 1,2-dioxygenase.

Accordingly, in one embodiment, the process of the present invention for producing threonine in a non-human organism, like a microorganism or a plant or a part thereof, comprises increasing or generating the activity of a gene product with the activity of a gene product conferring the activity of “2,3-dihydroxyphenylpropionate 1,2-dioxygenase”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B0348, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B0348, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B0348, or a functional equivalent or a homolog thereof as depicted in column 8 of Table II, application no. 4, preferably a homolog or functional equivalent as depicted in column 8 of Table II B, application no. 4, and being depicted in the same respective line as said B0348, and preferably the activity is increased plastidic.

Accordingly, in one embodiment, the molecule which activity is to be increased in the process of the invention is the gene product with an activity as a “2,3-dihydroxyphenylpropionate 1,2-dioxygenase”, preferably it is the molecule of section (a) or (b) of this paragraph.

In particular, it was observed that in plants, especially in Arabidopsis thaliana, increasing or generating the activity of a gene product plastidic with the activity of a “2,3-dihydroxyphenylpropionate 1,2-dioxygenase”, preferably being encoded by a gene comprising the nucleic acid sequence SEQ ID NO. 7269, preferably the coding region thereof, conferred the production of or the increase in threonine compared with the wild type control.

The nucleic acid sequence of B0449 from Escherichia coli, e.g. as shown in the respective line in column 5 of Table I, application no. 4, has been published in B. And the activity of the gene product thereof is the activity of ATP-binding component of a transport system.

Accordingly, in one embodiment, the process of the present invention for producing threonine in a non-human organism, like a microorganism or a plant or a part thereof, comprises increasing or generating the activity of a gene product with the activity of a gene product conferring the activity of “ATP-binding component of a transport system”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B0449, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B0449, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B0449, or a functional equivalent or a homolog thereof as depicted in column 8 of Table II, application no. 4, preferably a homolog or functional equivalent as depicted in column 8 of Table II B, application no. 4, and being depicted in the same respective line as said B0449, and preferably the activity is increased non-targeted.

Accordingly, in one embodiment, the molecule which activity is to be increased in the process of the invention is the gene product with an activity as a “ATP-binding component of a transport system”, preferably it is the molecule of section (a) or (b) of this paragraph.

In particular, it was observed that in plants, especially in Arabidopsis thaliana, increasing or generating the activity of a gene product non-targeted with the activity of a “ATP-binding component of a transport system”, preferably being encoded by a gene comprising the nucleic acid sequence SEQ ID NO. 7333, preferably the coding region thereof, conferred the production of or the increase in threonine compared with the wild type control.

The nucleic acid sequence of B0486 from Escherichia coli, e.g. as shown in the respective line in column 5 of Table I, application no. 4, has been published in B. And the activity of the gene product thereof is the activity of membrane transport protein.

Accordingly, in one embodiment, the process of the present invention for producing threonine in a non-human organism, like a microorganism or a plant or a part thereof, comprises increasing or generating the activity of a gene product with the activity of a gene product conferring the activity of “membrane transport protein”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B0486, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B0486, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B0486, or a functional equivalent or a homolog thereof as depicted in column 8 of Table II, application no. 4, preferably a homolog or functional equivalent as depicted in column 8 of Table II B, application no. 4, and being depicted in the same respective line as said B0486, and preferably the activity is increased non-targeted.

Accordingly, in one embodiment, the molecule which activity is to be increased in the process of the invention is the gene product with an activity as a “membrane transport protein”, preferably it is the molecule of section (a) or (b) of this paragraph.

In particular, it was observed that in plants, especially in Arabidopsis thaliana, increasing or generating the activity of a gene product non-targeted with the activity of a “membrane transport protein”, preferably being encoded by a gene comprising the nucleic acid sequence SEQ ID NO. 7686, preferably the coding region thereof, conferred the production of or the increase in threonine compared with the wild type control.

The nucleic acid sequence of B0593 from Escherichia coli, e.g. as shown in the respective line in column 5 of Table I, application no. 4, has been published in B. And the activity of the gene product thereof is the activity of isochorismate synthase.

Accordingly, in one embodiment, the process of the present invention for producing threonine in a non-human organism, like a microorganism or a plant or a part thereof, comprises increasing or generating the activity of a gene product with the activity of a gene product conferring the activity of “isochorismate synthase”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B0593, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B0593, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B0593, or a functional equivalent or a homolog thereof as depicted in column 8 of Table II, application no. 4, preferably a homolog or functional equivalent as depicted in column 8 of Table II B, application no. 4, and being depicted in the same respective line as said B0593, and preferably the activity is increased plastidic.

Accordingly, in one embodiment, the molecule which activity is to be increased in the process of the invention is the gene product with an activity as a “isochorismate synthase”, preferably it is the molecule of section (a) or (b) of this paragraph.

In particular, it was observed that in plants, especially in Arabidopsis thaliana, increasing or generating the activity of a gene product plastidic with the activity of a “isochorismate synthase”, preferably being encoded by a gene comprising the nucleic acid sequence SEQ ID NO. 35967, preferably the coding region thereof, conferred the production of or the increase in threonine compared with the wild type control.

The nucleic acid sequence of B0695 from Escherichia coli, e.g. as shown in the respective line in column 5 of Table I, application no. 4, has been published in B. And the activity of the gene product thereof is the activity of sensor histidine kinase.

Accordingly, in one embodiment, the process of the present invention for producing threonine in a non-human organism, like a microorganism or a plant or a part thereof, comprises increasing or generating the activity of a gene product with the activity of a gene product conferring the activity of “sensor histidine kinase”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B0695, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B0695, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B0695, or a functional equivalent or a homolog thereof as depicted in column 8 of Table II, application no. 4, preferably a homolog or functional equivalent as depicted in column 8 of Table II B, application no. 4, and being depicted in the same respective line as said B0695, and preferably the activity is increased non-targeted.

Accordingly, in one embodiment, the molecule which activity is to be increased in the process of the invention is the gene product with an activity as a “sensor histidine kinase”, preferably it is the molecule of section (a) or (b) of this paragraph.

In particular, it was observed that in plants, especially in Arabidopsis thaliana, increasing or generating the activity of a gene product non-targeted with the activity of a “sensor histidine kinase”, preferably being encoded by a gene comprising the nucleic acid sequence SEQ ID NO. 93678, preferably the coding region thereof, conferred the production of or the increase in threonine compared with the wild type control.

The nucleic acid sequence of B0728 from Escherichia coli, e.g. as shown in the respective line in column 5 of Table I, application no. 4, has been published in B. And the activity of the gene product thereof is the activity of succinyl-CoA synthetase beta chain.

Accordingly, in one embodiment, the process of the present invention for producing threonine in a non-human organism, like a microorganism or a plant or a part thereof, comprises increasing or generating the activity of a gene product with the activity of a gene product conferring the activity of “succinyl-CoA synthetase beta chain”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B0728, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B0728, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B0728, or a functional equivalent or a homolog thereof as depicted in column 8 of Table II, application no. 4, preferably a homolog or functional equivalent as depicted in column 8 of Table II B, application no. 4, and being depicted in the same respective line as said B0728, and preferably the activity is increased plastidic.

Accordingly, in one embodiment, the molecule which activity is to be increased in the process of the invention is the gene product with an activity as a “succinyl-CoA synthetase beta chain”, preferably it is the molecule of section (a) or (b) of this paragraph.

In particular, it was observed that in plants, especially in Arabidopsis thaliana, increasing or generating the activity of a gene product plastidic with the activity of a “succinyl-CoA synthetase beta chain”, preferably being encoded by a gene comprising the nucleic acid sequence SEQ ID NO. 93793, preferably the coding region thereof, conferred the production of or the increase in threonine compared with the wild type control.

The nucleic acid sequence of B0898 from Escherichia coli, e.g. as shown in the respective line in column 5 of Table I, application no. 4, has been published in B. And the activity of the gene product thereof is the activity of major facilitator superfamily transporter protein.

Accordingly, in one embodiment, the process of the present invention for producing threonine in a non-human organism, like a microorganism or a plant or a part thereof, comprises increasing or generating the activity of a gene product with the activity of a gene product conferring the activity of “major facilitator superfamily transporter protein”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B0898, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B0898, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B0898, or a functional equivalent or a homolog thereof as depicted in column 8 of Table II, application no. 4, preferably a homolog or functional equivalent as depicted in column 8 of Table II B, application no. 4, and being depicted in the same respective line as said B0898, and preferably the activity is increased non-targeted.

Accordingly, in one embodiment, the molecule which activity is to be increased in the process of the invention is the gene product with an activity as a “major facilitator superfamily transporter protein”, preferably it is the molecule of section (a) or (b) of this paragraph.

In particular, it was observed that in plants, especially in Arabidopsis thaliana, increasing or generating the activity of a gene product non-targeted with the activity of a “major facilitator superfamily transporter protein”, preferably being encoded by a gene comprising the nucleic acid sequence SEQ ID NO. 7917, preferably the coding region thereof, conferred the production of or the increase in threonine compared with the wild type control.

The nucleic acid sequence of B0980 from Escherichia coli, e.g. as shown in the respective line in column 5 of Table I, application no. 4, has been published in B. And the activity of the gene product thereof is the activity of phosphoanhydride phosphorylase.

Accordingly, in one embodiment, the process of the present invention for producing threonine in a non-human organism, like a microorganism or a plant or a part thereof, comprises increasing or generating the activity of a gene product with the activity of a gene product conferring the activity of “phosphoanhydride phosphorylase”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B0980, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B0980, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B0980, or a functional equivalent or a homolog thereof as depicted in column 8 of Table II, application no. 4, preferably a homolog or functional equivalent as depicted in column 8 of Table II B, application no. 4, and being depicted in the same respective line as said B0980, and preferably the activity is increased non-targeted.

Accordingly, in one embodiment, the molecule which activity is to be increased in the process of the invention is the gene product with an activity as a “phosphoanhydride phosphorylase”, preferably it is the molecule of section (a) or (b) of this paragraph.

In particular, it was observed that in plants, especially in Arabidopsis thaliana, increasing or generating the activity of a gene product non-targeted with the activity of a “phosphoanhydride phosphorylase”, preferably being encoded by a gene comprising the nucleic acid sequence SEQ ID NO. 36809, preferably the coding region thereof, conferred the production of or the increase in threonine compared with the wild type control.

The nucleic acid sequence of B1003 from Escherichia coli, e.g. as shown in the respective line in column 5 of Table I, application no. 4, has been published in B. And the activity of the gene product thereof is the activity of b1003-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “b1003-protein”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B1003, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B1003, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B1003, or a functional equivalent or a homolog thereof as depicted in column 8 of Table II, application no. 4, preferably a homolog or functional equivalent as depicted in column 8 of Table II B, application no. 4, and being depicted in the same respective line as said B1003, and preferably the activity is increased non-targeted.

Accordingly, in one embodiment, the molecule which activity is to be increased in the process of the invention is the gene product with an activity as a “b1003-protein”, preferably it is the molecule of section (a) or (b) of this paragraph.

In particular, it was observed that in plants, especially in Arabidopsis thaliana, increasing or generating the activity of a gene product non-targeted with the activity of a “b1003-protein”, preferably being encoded by a gene comprising the nucleic acid sequence SEQ ID NO. 7941, preferably the coding region thereof, conferred the production of or the increase in threonine compared with the wild type control.

The nucleic acid sequence of B1108 from Escherichia coli, e.g. as shown in the respective line in column 5 of Table I, application no. 4, has been published in B. And the activity of the gene product thereof is the activity of b1108-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “b1108-protein”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B1108, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B1108, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B1108, or a functional equivalent or a homolog thereof as depicted in column 8 of Table II, application no. 4, preferably a homolog or functional equivalent as depicted in column 8 of Table II B, application no. 4, and being depicted in the same respective line as said B1108, and preferably the activity is increased non-targeted.

Accordingly, in one embodiment, the molecule which activity is to be increased in the process of the invention is the gene product with an activity as a “b1108-protein”, preferably it is the molecule of section (a) or (b) of this paragraph.

In particular, it was observed that in plants, especially in Arabidopsis thaliana, increasing or generating the activity of a gene product non-targeted with the activity of a “b1108-protein”, preferably being encoded by a gene comprising the nucleic acid sequence SEQ ID NO. 36937, preferably the coding region thereof, conferred the production of or the increase in threonine compared with the wild type control.

The nucleic acid sequence of B1348 from Escherichia coli, e.g. as shown in the respective line in column 5 of Table I, application no. 4, has been published in B. And the activity of the gene product thereof is the activity of restriction alleviation protein.

Accordingly, in one embodiment, the process of the present invention for producing threonine in a non-human organism, like a microorganism or a plant or a part thereof, comprises increasing or generating the activity of a gene product with the activity of a gene product conferring the activity of “restriction alleviation protein”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B1348, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B1348, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B1348, or a functional equivalent or a homolog thereof as depicted in column 8 of Table II, application no. 4, preferably a homolog or functional equivalent as depicted in column 8 of Table II B, application no. 4, and being depicted in the same respective line as said B1348, and preferably the activity is increased non-targeted.

Accordingly, in one embodiment, the molecule which activity is to be increased in the process of the invention is the gene product with an activity as a “restriction alleviation protein”, preferably it is the molecule of section (a) or (b) of this paragraph.

In particular, it was observed that in plants, especially in Arabidopsis thaliana, increasing or generating the activity of a gene product non-targeted with the activity of a “restriction alleviation protein”, preferably being encoded by a gene comprising the nucleic acid sequence SEQ ID NO. 77768, preferably the coding region thereof, conferred the production of or the increase in threonine compared with the wild type control. The nucleic acid sequence of B1445 from Escherichia coli, e.g. as shown in the respective line in column 5 of Table I, application no. 4, has been published in B. And the activity of the gene product thereof is the activity of b1445-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “b1445-protein”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B1445, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B1445, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B1445, or a functional equivalent or a homolog thereof as depicted in column 8 of Table II, application no. 4, preferably a homolog or functional equivalent as depicted in column 8 of Table II B, application no. 4, and being depicted in the same respective line as said B1445, and preferably the activity is increased non-targeted.

Accordingly, in one embodiment, the molecule which activity is to be increased in the process of the invention is the gene product with an activity as a “b1445-protein”, preferably it is the molecule of section (a) or (b) of this paragraph.

In particular, it was observed that in plants, especially in Arabidopsis thaliana, increasing or generating the activity of a gene product non-targeted with the activity of a “b1445-protein”, preferably being encoded by a gene comprising the nucleic acid sequence SEQ ID NO. 38289, preferably the coding region thereof, conferred the production of or the increase in threonine compared with the wild type control.

The nucleic acid sequence of B1479 from Escherichia coli, e.g. as shown in the respective line in column 5 of Table I, application no. 4, has been published in B. And the activity of the gene product thereof is the activity of NADP-dependent malic enzyme.

Accordingly, in one embodiment, the process of the present invention for producing threonine in a non-human organism, like a microorganism or a plant or a part thereof, comprises increasing or generating the activity of a gene product with the activity of a gene product conferring the activity of “NADP-dependent malic enzyme”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B1479, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B1479, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B1479, or a functional equivalent or a homolog thereof as depicted in column 8 of Table II, application no. 4, preferably a homolog or functional equivalent as depicted in column 8 of Table II B, application no. 4, and being depicted in the same respective line as said B1479, and preferably the activity is increased non-targeted.

Accordingly, in one embodiment, the molecule which activity is to be increased in the process of the invention is the gene product with an activity as a “NADP-dependent malic enzyme”, preferably it is the molecule of section (a) or (b) of this paragraph.

In particular, it was observed that in plants, especially in Arabidopsis thaliana, increasing or generating the activity of a gene product non-targeted with the activity of a “NADP-dependent malic enzyme”, preferably being encoded by a gene comprising the nucleic acid sequence SEQ ID NO. 77774, preferably the coding region thereof, conferred the production of or the increase in threonine compared with the wild type control.

The nucleic acid sequence of B1522 from Escherichia coli, e.g. as shown in the respective line in column 5 of Table I, application no. 4, has been published in B. And the activity of the gene product thereof is the activity of b1522-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “b1522-protein”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B1522, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B1522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B1522, or a functional equivalent or a homolog thereof as depicted in column 8 of Table II, application no. 4, preferably a homolog or functional equivalent as depicted in column 8 of Table II B, application no. 4, and being depicted in the same respective line as said B1522, and preferably the activity is increased non-targeted.

Accordingly, in one embodiment, the molecule which activity is to be increased in the process of the invention is the gene product with an activity as a “b1522-protein”, preferably it is the molecule of section (a) or (b) of this paragraph.

In particular, it was observed that in plants, especially in Arabidopsis thaliana, increasing or generating the activity of a gene product non-targeted with the activity of a “b1522-protein”, preferably being encoded by a gene comprising the nucleic acid sequence SEQ ID NO. 7947, preferably the coding region thereof, conferred the production of or the increase in threonine compared with the wild type control.

The nucleic acid sequence of B1670 from Escherichia coli, e.g. as shown in the respective line in column 5 of Table I, application no. 4, has been published in B. And the activity of the gene product thereof is the activity of b1670-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “b1670-protein”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B1670, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B1670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B1670, or a functional equivalent or a homolog thereof as depicted in column 8 of Table II, application no. 4, preferably a homolog or functional equivalent as depicted in column 8 of Table II B, application no. 4, and being depicted in the same respective line as said B1670, and preferably the activity is increased non-targeted.

Accordingly, in one embodiment, the molecule which activity is to be increased in the process of the invention is the gene product with an activity as a “b1670-protein”, preferably it is the molecule of section (a) or (b) of this paragraph.

In particular, it was observed that in plants, especially in Arabidopsis thaliana, increasing or generating the activity of a gene product non-targeted with the activity of a “b1670-protein”, preferably being encoded by a gene comprising the nucleic acid sequence SEQ ID NO. 78753, preferably the coding region thereof, conferred the production of or the increase in threonine compared with the wild type control.

The nucleic acid sequence of B1854 from Escherichia coli, e.g. as shown in the respective line in column 5 of Table I, application no. 4, has been published in B. And the activity of the gene product thereof is the activity of pyruvate kinase.

Accordingly, in one embodiment, the process of the present invention for producing threonine in a non-human organism, like a microorganism or a plant or a part thereof, comprises increasing or generating the activity of a gene product with the activity of a gene product conferring the activity of “pyruvate kinase”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B1854, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B1854, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B1854, or a functional equivalent or a homolog thereof as depicted in column 8 of Table II, application no. 4, preferably a homolog or functional equivalent as depicted in column 8 of Table II B, application no. 4, and being depicted in the same respective line as said B1854, and preferably the activity is increased plastidic.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 8363, preferably the coding region thereof, conferred the production of or the increase in threonine compared with the wild type control.

The nucleic acid sequence of B1930 from Escherichia coli, e.g. as shown in the respective line in column 5 of Table I, application no. 4, has been published in B. And the activity of the gene product thereof is the activity of b1930-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “b1930-protein”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B1930, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B1930, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B1930, or a functional equivalent or a homolog thereof as depicted in column 8 of Table II, application no. 4, preferably a homolog or functional equivalent as depicted in column 8 of Table II B, application no. 4, and being depicted in the same respective line as said B1930, and preferably the activity is increased non-targeted.

Accordingly, in one embodiment, the molecule which activity is to be increased in the process of the invention is the gene product with an activity as a “b1930-protein”, preferably it is the molecule of section (a) or (b) of this paragraph.

In particular, it was observed that in plants, especially in Arabidopsis thaliana, increasing or generating the activity of a gene product non-targeted with the activity of a “b1930-protein”, preferably being encoded by a gene comprising the nucleic acid sequence SEQ ID NO. 79181, preferably the coding region thereof, conferred the production of or the increase in threonine compared with the wild type control.

The nucleic acid sequence of B2063 from Escherichia coli, e.g. as shown in the respective line in column 5 of Table I, application no. 4, has been published in B. And the activity of the gene product thereof is the activity of transport protein.

Accordingly, in one embodiment, the process of the present invention for producing threonine in a non-human organism, like a microorganism or a plant or a part thereof, comprises increasing or generating the activity of a gene product with the activity of a gene product conferring the activity of “transport protein”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B2063, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B2063, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B2063, or a functional equivalent or a homolog thereof as depicted in column 8 of Table II, application no. 4, preferably a homolog or functional equivalent as depicted in column 8 of Table II B, application no. 4, and being depicted in the same respective line as said B2063, and preferably the activity is increased non-targeted.

Accordingly, in one embodiment, the molecule which activity is to be increased in the process of the invention is the gene product with an activity as a “transport protein”, preferably it is the molecule of section (a) or (b) of this paragraph.

In particular, it was observed that in plants, especially in Arabidopsis thaliana, increasing or generating the activity of a gene product non-targeted with the activity of a “transport protein”, preferably being encoded by a gene comprising the nucleic acid sequence SEQ ID NO. 38947, preferably the coding region thereof, conferred the production of or the increase in threonine compared with the wild type control.

The nucleic acid sequence of B2360 from Escherichia coli, e.g. as shown in the respective line in column 5 of Table I, application no. 4, has been published in B. And the activity of the gene product thereof is the activity of b2360-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “b2360-protein”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B2360, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B2360, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B2360, or a functional equivalent or a homolog thereof as depicted in column 8 of Table II, application no. 4, preferably a homolog or functional equivalent as depicted in column 8 of Table II B, application no. 4, and being depicted in the same respective line as said B2360, and preferably the activity is increased non-targeted.

Accordingly, in one embodiment, the molecule which activity is to be increased in the process of the invention is the gene product with an activity as a “b2360-protein”, preferably it is the molecule of section (a) or (b) of this paragraph.

In particular, it was observed that in plants, especially in Arabidopsis thaliana, increasing or generating the activity of a gene product non-targeted with the activity of a “b2360-protein”, preferably being encoded by a gene comprising the nucleic acid sequence SEQ ID NO. 39219, preferably the coding region thereof, conferred the production of or the increase in threonine compared with the wild type control.

The nucleic acid sequence of B2366 from Escherichia coli, e.g. as shown in the respective line in column 5 of Table I, application no. 4, has been published in B. And the activity of the gene product thereof is the activity of D-serine dehydratase.

Accordingly, in one embodiment, the process of the present invention for producing threonine in a non-human organism, like a microorganism or a plant or a part thereof, comprises increasing or generating the activity of a gene product with the activity of a gene product conferring the activity of “D-serine dehydratase”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B2366, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B2366, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B2366, or a functional equivalent or a homolog thereof as depicted in column 8 of Table II, application no. 4, preferably a homolog or functional equivalent as depicted in column 8 of Table II B, application no. 4, and being depicted in the same respective line as said B2366, and preferably the activity is increased plastidic.

Accordingly, in one embodiment, the molecule which activity is to be increased in the process of the invention is the gene product with an activity as a “D-serine dehydratase”, preferably it is the molecule of section (a) or (b) of this paragraph.

In particular, it was observed that in plants, especially in Arabidopsis thaliana, increasing or generating the activity of a gene product plastidic with the activity of a “D-serine dehydratase”, preferably being encoded by a gene comprising the nucleic acid sequence SEQ ID NO. 79342, preferably the coding region thereof, conferred the production of or the increase in threonine compared with the wild type control.

The nucleic acid sequence of B2399 from Escherichia coli, e.g. as shown in the respective line in column 5 of Table I, application no. 4, has been published in B. And the activity of the gene product thereof is the activity of b2399-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “b2399-protein”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B2399, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B2399, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B2399, or a functional equivalent or a homolog thereof as depicted in column 8 of Table II, application no. 4, preferably a homolog or functional equivalent as depicted in column 8 of Table II B, application no. 4, and being depicted in the same respective line as said B2399, and preferably the activity is increased non-targeted.

Accordingly, in one embodiment, the molecule which activity is to be increased in the process of the invention is the gene product with an activity as a “b2399-protein”, preferably it is the molecule of section (a) or (b) of this paragraph.

In particular, it was observed that in plants, especially in Arabidopsis thaliana, increasing or generating the activity of a gene product non-targeted with the activity of a “b2399-protein”, preferably being encoded by a gene comprising the nucleic acid sequence SEQ ID NO. 39237, preferably the coding region thereof, conferred the production of or the increase in threonine compared with the wild type control.

The nucleic acid sequence of B2405 from Escherichia coli, e.g. as shown in the respective line in column 5 of Table I, application no. 4, has been published in B. And the activity of the gene product thereof is the activity of transcriptional regulator.

Accordingly, in one embodiment, the process of the present invention for producing threonine in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B2405, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B2405, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B2405, or a functional equivalent or a homolog thereof as depicted in column 8 of Table II, application no. 4, preferably a homolog or functional equivalent as depicted in column 8 of Table II B, application no. 4, and being depicted in the same respective line as said B2405, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 39255, preferably the coding region thereof, conferred the production of or the increase in threonine compared with the wild type control.

The nucleic acid sequence of B2513 from Escherichia coli, e.g. as shown in the respective line in column 5 of Table I, application no. 4, has been published in B. And the activity of the gene product thereof is the activity of b2513-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “b2513-protein”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B2513, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B2513, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B2513, or a functional equivalent or a homolog thereof as depicted in column 8 of Table II, application no. 4, preferably a homolog or functional equivalent as depicted in column 8 of Table II B, application no. 4, and being depicted in the same respective line as said B2513, and preferably the activity is increased non-targeted.

Accordingly, in one embodiment, the molecule which activity is to be increased in the process of the invention is the gene product with an activity as a “b2513-protein”, preferably it is the molecule of section (a) or (b) of this paragraph.

In particular, it was observed that in plants, especially in Arabidopsis thaliana, increasing or generating the activity of a gene product non-targeted with the activity of a “b2513-protein”, preferably being encoded by a gene comprising the nucleic acid sequence SEQ ID NO. 9167, preferably the coding region thereof, conferred the production of or the increase in threonine compared with the wild type control.

The nucleic acid sequence of B2613 from Escherichia coli, e.g. as shown in the respective line in column 5 of Table I, application no. 4, has been published in B. And the activity of the gene product thereof is the activity of b2613-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “b2613-protein”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B2613, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B2613, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B2613, or a functional equivalent or a homolog thereof as depicted in column 8 of Table II, application no. 4, preferably a homolog or functional equivalent as depicted in column 8 of Table II B, application no. 4, and being depicted in the same respective line as said B2613, and preferably the activity is increased non-targeted.

Accordingly, in one embodiment, the molecule which activity is to be increased in the process of the invention is the gene product with an activity as a “b2613-protein”, preferably it is the molecule of section (a) or (b) of this paragraph.

In particular, it was observed that in plants, especially in Arabidopsis thaliana, increasing or generating the activity of a gene product non-targeted with the activity of a “b2613-protein”, preferably being encoded by a gene comprising the nucleic acid sequence SEQ ID NO. 40665, preferably the coding region thereof, conferred the production of or the increase in threonine compared with the wild type control.

The nucleic acid sequence of B2701 from Escherichia coli, e.g. as shown in the respective line in column 5 of Table I, application no. 4, has been published in B. And the activity of the gene product thereof is the activity of murein transglycosylase.

Accordingly, in one embodiment, the process of the present invention for producing threonine in a non-human organism, like a microorganism or a plant or a part thereof, comprises increasing or generating the activity of a gene product with the activity of a gene product conferring the activity of “murein transglycosylase”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B2701, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B2701, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B2701, or a functional equivalent or a homolog thereof as depicted in column 8 of Table II, application no. 4, preferably a homolog or functional equivalent as depicted in column 8 of Table II B, application no. 4, and being depicted in the same respective line as said B2701, and preferably the activity is increased non-targeted.

Accordingly, in one embodiment, the molecule which activity is to be increased in the process of the invention is the gene product with an activity as a “murein transglycosylase”, preferably it is the molecule of section (a) or (b) of this paragraph.

In particular, it was observed that in plants, especially in Arabidopsis thaliana, increasing or generating the activity of a gene product non-targeted with the activity of a “murein transglycosylase”, preferably being encoded by a gene comprising the nucleic acid sequence SEQ ID NO. 80756, preferably the coding region thereof, conferred the production of or the increase in threonine compared with the wild type control.

The nucleic acid sequence of B2714 from Escherichia coli, e.g. as shown in the respective line in column 5 of Table I, application no. 4, has been published in B. And the activity of the gene product thereof is the activity of transcriptional regulator.

Accordingly, in one embodiment, the process of the present invention for producing threonine in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B2714, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B2714, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B2714, or a functional equivalent or a homolog thereof as depicted in column 8 of Table II, application no. 4, preferably a homolog or functional equivalent as depicted in column 8 of Table II B, application no. 4, and being depicted in the same respective line as said B2714, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 40741, preferably the coding region thereof, conferred the production of or the increase in threonine compared with the wild type control.

The nucleic acid sequence of B2739 from Escherichia coli, e.g. as shown in the respective line in column 5 of Table I, application no. 4, has been published in B. And the activity of the gene product thereof is the activity of b2739-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “b2739-protein”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B2739, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B2739, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B2739, or a functional equivalent or a homolog thereof as depicted in column 8 of Table II, application no. 4, preferably a homolog or functional equivalent as depicted in column 8 of Table II B, application no. 4, and being depicted in the same respective line as said B2739, and preferably the activity is increased non-targeted.

Accordingly, in one embodiment, the molecule which activity is to be increased in the process of the invention is the gene product with an activity as a “b2739-protein”, preferably it is the molecule of section (a) or (b) of this paragraph.

In particular, it was observed that in plants, especially in Arabidopsis thaliana, increasing or generating the activity of a gene product non-targeted with the activity of a “b2739-protein”, preferably being encoded by a gene comprising the nucleic acid sequence SEQ ID NO. 80906, preferably the coding region thereof, conferred the production of or the increase in threonine compared with the wild type control.

The nucleic acid sequence of B2747 from Escherichia coli, e.g. as shown in the respective line in column 5 of Table I, application no. 4, has been published in B. And the activity of the gene product thereof is the activity of 2-C-methyl-D-erythritol 4-phosphate cytidylyltransferase.

Accordingly, in one embodiment, the process of the present invention for producing threonine in a non-human organism, like a microorganism or a plant or a part thereof, comprises increasing or generating the activity of a gene product with the activity of a gene product conferring the activity of “2-C-methyl-D-erythritol 4-phosphate cytidylyltransferase”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B2747, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B2747, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B2747, or a functional equivalent or a homolog thereof as depicted in column 8 of Table II, application no. 4, preferably a homolog or functional equivalent as depicted in column 8 of Table II B, application no. 4, and being depicted in the same respective line as said B2747, and preferably the activity is increased non-targeted.

Accordingly, in one embodiment, the molecule which activity is to be increased in the process of the invention is the gene product with an activity as a “2-C-methyl-D-erythritol 4-phosphate cytidylyltransferase”, preferably it is the molecule of section (a) or (b) of this paragraph.

In particular, it was observed that in plants, especially in Arabidopsis thaliana, increasing or generating the activity of a gene product non-targeted with the activity of a “2-C-methyl-Derythritol 4-phosphate cytidylyltransferase”, preferably being encoded by a gene comprising the nucleic acid sequence SEQ ID NO. 81075, preferably the coding region thereof, conferred the production of or the increase in threonine compared with the wild type control.

The nucleic acid sequence of B2812 from Escherichia coli, e.g. as shown in the respective line in column 5 of Table I, application no. 4, has been published in B. And the activity of the gene product thereof is the activity of b2812-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “b2812-protein”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B2812, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B2812, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B2812, or a functional equivalent or a homolog thereof as depicted in column 8 of Table II, application no. 4, preferably a homolog or functional equivalent as depicted in column 8 of Table II B, application no. 4, and being depicted in the same respective line as said B2812, and preferably the activity is increased plastidic.

Accordingly, in one embodiment, the molecule which activity is to be increased in the process of the invention is the gene product with an activity as a “b2812-protein”, preferably it is the molecule of section (a) or (b) of this paragraph.

In particular, it was observed that in plants, especially in Arabidopsis thaliana, increasing or generating the activity of a gene product plastidic with the activity of a “b2812-protein”, preferably being encoded by a gene comprising the nucleic acid sequence SEQ ID NO. 40795, preferably the coding region thereof, conferred the production of or the increase in threonine compared with the wild type control.

The nucleic acid sequence of B2812 from Escherichia coli, e.g. as shown in the respective line in column 5 of Table I, application no. 4, has been published in B. And the activity of the gene product thereof is the activity of b2812-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “b2812-protein”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B2812, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B2812, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B2812, or a functional equivalent or a homolog thereof as depicted in column 8 of Table II, application no. 4, preferably a homolog or functional equivalent as depicted in column 8 of Table II B, application no. 4, and being depicted in the same respective line as said B2812, and preferably the activity is increased non-targeted.

Accordingly, in one embodiment, the molecule which activity is to be increased in the process of the invention is the gene product with an activity as a “b2812-protein”, preferably it is the molecule of section (a) or (b) of this paragraph.

In particular, it was observed that in plants, especially in Arabidopsis thaliana, increasing or generating the activity of a gene product non-targeted with the activity of a “b2812-protein”, preferably being encoded by a gene comprising the nucleic acid sequence SEQ ID NO. 40795, preferably the coding region thereof, conferred the production of or the increase in threonine compared with the wild type control.

The nucleic acid sequence of B2913 from Escherichia coli, e.g. as shown in the respective line in column 5 of Table I, application no. 4, has been published in B. And the activity of the gene product thereof is the activity of D-3-phosphoglycerate dehydrogenase.

Accordingly, in one embodiment, the process of the present invention for producing threonine in a non-human organism, like a microorganism or a plant or a part thereof, comprises increasing or generating the activity of a gene product with the activity of a gene product conferring the activity of “D-3-phosphoglycerate dehydrogenase”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B2913, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B2913, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B2913, or a functional equivalent or a homolog thereof as depicted in column 8 of Table II, application no. 4, preferably a homolog or functional equivalent as depicted in column 8 of Table II B, application no. 4, and being depicted in the same respective line as said B2913, and preferably the activity is increased plastidic.

Accordingly, in one embodiment, the molecule which activity is to be increased in the process of the invention is the gene product with an activity as a “D-3-phosphoglycerate dehydrogenase”, preferably it is the molecule of section (a) or (b) of this paragraph.

In particular, it was observed that in plants, especially in Arabidopsis thaliana, increasing or generating the activity of a gene product plastidic with the activity of a “D-3-phosphoglycerate dehydrogenase”, preferably being encoded by a gene comprising the nucleic acid sequence SEQ ID NO. 81988, preferably the coding region thereof, conferred the production of or the increase in threonine compared with the wild type control.

The nucleic acid sequence of B2957 from Escherichia coli, e.g. as shown in the respective line in column 5 of Table I, application no. 4, has been published in B. And the activity of the gene product thereof is the activity of L-asparaginase.

Accordingly, in one embodiment, the process of the present invention for producing threonine in a non-human organism, like a microorganism or a plant or a part thereof, comprises increasing or generating the activity of a gene product with the activity of a gene product conferring the activity of “L-asparaginase”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B2957, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B2957, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B2957, or a functional equivalent or a homolog thereof as depicted in column 8 of Table II, application no. 4, preferably a homolog or functional equivalent as depicted in column 8 of Table II B, application no. 4, and being depicted in the same respective line as said B2957, and preferably the activity is increased non-targeted.

Accordingly, in one embodiment, the molecule which activity is to be increased in the process of the invention is the gene product with an activity as a “L-asparaginase”, preferably it is the molecule of section (a) or (b) of this paragraph.

In particular, it was observed that in plants, especially in Arabidopsis thaliana, increasing or generating the activity of a gene product non-targeted with the activity of a “L-asparaginase”, preferably being encoded by a gene comprising the nucleic acid sequence SEQ ID NO. 41499, preferably the coding region thereof, conferred the production of or the increase in threonine compared with the wild type control.

The nucleic acid sequence of B3064 from Escherichia coli, e.g. as shown in the respective line in column 5 of Table I, application no. 4, has been published in B. And the activity of the gene product thereof is the activity of glycoprotease.

Accordingly, in one embodiment, the process of the present invention for producing threonine in a non-human organism, like a microorganism or a plant or a part thereof, comprises increasing or generating the activity of a gene product with the activity of a gene product conferring the activity of “glycoprotease”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B3064, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B3064, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B3064, or a functional equivalent or a homolog thereof as depicted in column 8 of Table II, application no. 4, preferably a homolog or functional equivalent as depicted in column 8 of Table II B, application no. 4, and being depicted in the same respective line as said B3064, and preferably the activity is increased non-targeted.

Accordingly, in one embodiment, the molecule which activity is to be increased in the process of the invention is the gene product with an activity as a “glycoprotease”, preferably it is the molecule of section (a) or (b) of this paragraph.

In particular, it was observed that in plants, especially in Arabidopsis thaliana, increasing or generating the activity of a gene product non-targeted with the activity of a “glycoprotease”, preferably being encoded by a gene comprising the nucleic acid sequence SEQ ID NO. 42046, preferably the coding region thereof, conferred the production of or the increase in threonine compared with the wild type control.

The nucleic acid sequence of B3083 from Escherichia coli, e.g. as shown in the respective line in column 5 of Table I, application no. 4, has been published in B. And the activity of the gene product thereof is the activity of b3083-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “b3083-protein”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B3083, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B3083, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B3083, or a functional equivalent or a homolog thereof as depicted in column 8 of Table II, application no. 4, preferably a homolog or functional equivalent as depicted in column 8 of Table II B, application no. 4, and being depicted in the same respective line as said B3083, and preferably the activity is increased non-targeted.

Accordingly, in one embodiment, the molecule which activity is to be increased in the process of the invention is the gene product with an activity as a “b3083-protein”, preferably it is the molecule of section (a) or (b) of this paragraph.

In particular, it was observed that in plants, especially in Arabidopsis thaliana, increasing or generating the activity of a gene product non-targeted with the activity of a “b3083-protein”, preferably being encoded by a gene comprising the nucleic acid sequence SEQ ID NO. 82424, preferably the coding region thereof, conferred the production of or the increase in threonine compared with the wild type control.

The nucleic acid sequence of B3262 from Escherichia coli, e.g. as shown in the respective line in column 5 of Table I, application no. 4, has been published in B. And the activity of the gene product thereof is the activity of methyltransferase.

Accordingly, in one embodiment, the process of the present invention for producing threonine in a non-human organism, like a microorganism or a plant or a part thereof, comprises increasing or generating the activity of a gene product with the activity of a gene product conferring the activity of “methyltransferase”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B3262, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B3262, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B3262, or a functional equivalent or a homolog thereof as depicted in column 8 of Table II, application no. 4, preferably a homolog or functional equivalent as depicted in column 8 of Table II B, application no. 4, and being depicted in the same respective line as said B3262, and preferably the activity is increased non-targeted.

Accordingly, in one embodiment, the molecule which activity is to be increased in the process of the invention is the gene product with an activity as a “methyltransferase”, preferably it is the molecule of section (a) or (b) of this paragraph.

In particular, it was observed that in plants, especially in Arabidopsis thaliana, increasing or generating the activity of a gene product non-targeted with the activity of a “methyltransferase”, preferably being encoded by a gene comprising the nucleic acid sequence SEQ ID NO. 42502, preferably the coding region thereof, conferred the production of or the increase in threonine compared with the wild type control.

The nucleic acid sequence of B3429 from Escherichia coli, e.g. as shown in the respective line in column 5 of Table I, application no. 4, has been published in B. And the activity of the gene product thereof is the activity of glycogen synthase.

Accordingly, in one embodiment, the process of the present invention for producing threonine in a non-human organism, like a microorganism or a plant or a part thereof, comprises increasing or generating the activity of a gene product with the activity of a gene product conferring the activity of “glycogen synthase”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B3429, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B3429, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B3429, or a functional equivalent or a homolog thereof as depicted in column 8 of Table II, application no. 4, preferably a homolog or functional equivalent as depicted in column 8 of Table II B, application no. 4, and being depicted in the same respective line as said B3429, and preferably the activity is increased mitochondrial.

Accordingly, in one embodiment, the molecule which activity is to be increased in the process of the invention is the gene product with an activity as a “glycogen synthase”, preferably it is the molecule of section (a) or (b) of this paragraph.

In particular, it was observed that in plants, especially in Arabidopsis thaliana, increasing or generating the activity of a gene product mitochondrial with the activity of a “glycogen synthase”, preferably being encoded by a gene comprising the nucleic acid sequence SEQ ID NO. 94051, preferably the coding region thereof, conferred the production of or the increase in threonine compared with the wild type control.

The nucleic acid sequence of B3442 from Escherichia coli, e.g. as shown in the respective line in column 5 of Table I, application no. 4, has been published in B. And the activity of the gene product thereof is the activity of b3442-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “b3442-protein”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B3442, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B3442, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B3442, or a functional equivalent or a homolog thereof as depicted in column 8 of Table II, application no. 4, preferably a homolog or functional equivalent as depicted in column 8 of Table II B, application no. 4, and being depicted in the same respective line as said B3442, and preferably the activity is increased non-targeted.

Accordingly, in one embodiment, the molecule which activity is to be increased in the process of the invention is the gene product with an activity as a “b3442-protein”, preferably it is the molecule of section (a) or (b) of this paragraph.

In particular, it was observed that in plants, especially in Arabidopsis thaliana, increasing or generating the activity of a gene product non-targeted with the activity of a “b3442-protein”, preferably being encoded by a gene comprising the nucleic acid sequence SEQ ID NO. 94380, preferably the coding region thereof, conferred the production of or the increase in threonine compared with the wild type control.

The nucleic acid sequence of B3572 from Escherichia coli, e.g. as shown in the respective line in column 5 of Table I, application no. 4, has been published in B. And the activity of the gene product thereof is the activity of valine-pyruvate transaminase.

Accordingly, in one embodiment, the process of the present invention for producing threonine in a non-human organism, like a microorganism or a plant or a part thereof, comprises increasing or generating the activity of a gene product with the activity of a gene product conferring the activity of “valine-pyruvate transaminase”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B3572, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B3572, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B3572, or a functional equivalent or a homolog thereof as depicted in column 8 of Table II, application no. 4, preferably a homolog or functional equivalent as depicted in column 8 of Table II B, application no. 4, and being depicted in the same respective line as said B3572, and preferably the activity is increased plastidic.

Accordingly, in one embodiment, the molecule which activity is to be increased in the process of the invention is the gene product with an activity as a “valine-pyruvate transaminase”, preferably it is the molecule of section (a) or (b) of this paragraph.

In particular, it was observed that in plants, especially in Arabidopsis thaliana, increasing or generating the activity of a gene product plastidic with the activity of a “valine-pyruvate transaminase”, preferably being encoded by a gene comprising the nucleic acid sequence SEQ ID NO. 10172, preferably the coding region thereof, conferred the production of or the increase in threonine compared with the wild type control.

The nucleic acid sequence of B3646 from Escherichia coli, e.g. as shown in the respective line in column 5 of Table I, application no. 4, has been published in B. And the activity of the gene product thereof is the activity of b3646-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “b3646-protein”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B3646, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B3646, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B3646, or a functional equivalent or a homolog thereof as depicted in column 8 of Table II, application no. 4, preferably a homolog or functional equivalent as depicted in column 8 of Table II B, application no. 4, and being depicted in the same respective line as said B3646, and preferably the activity is increased non-targeted.

Accordingly, in one embodiment, the molecule which activity is to be increased in the process of the invention is the gene product with an activity as a “b3646-protein”, preferably it is the molecule of section (a) or (b) of this paragraph.

In particular, it was observed that in plants, especially in Arabidopsis thaliana, increasing or generating the activity of a gene product non-targeted with the activity of a “b3646-protein”, preferably being encoded by a gene comprising the nucleic acid sequence SEQ ID NO. 83429, preferably the coding region thereof, conferred the production of or the increase in threonine compared with the wild type control.

The nucleic acid sequence of B3732 from Escherichia coli, e.g. as shown in the respective line in column 5 of Table I, application no. 4, has been published in B. And the activity of the gene product thereof is the activity of ATP synthase subunit beta.

Accordingly, in one embodiment, the process of the present invention for producing threonine in a non-human organism, like a microorganism or a plant or a part thereof, comprises increasing or generating the activity of a gene product with the activity of a gene product conferring the activity of “ATP synthase subunit beta”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B3732, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B3732, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B3732, or a functional equivalent or a homolog thereof as depicted in column 8 of Table II, application no. 4, preferably a homolog or functional equivalent as depicted in column 8 of Table II B, application no. 4, and being depicted in the same respective line as said B3732, and preferably the activity is increased non-targeted.

Accordingly, in one embodiment, the molecule which activity is to be increased in the process of the invention is the gene product with an activity as a “ATP synthase subunit beta”, preferably it is the molecule of section (a) or (b) of this paragraph.

In particular, it was observed that in plants, especially in Arabidopsis thaliana, increasing or generating the activity of a gene product non-targeted with the activity of a “ATP synthase subunit beta”, preferably being encoded by a gene comprising the nucleic acid sequence SEQ ID NO. 43248, preferably the coding region thereof, conferred the production of or the increase in threonine compared with the wild type control.

The nucleic acid sequence of B3771 from Escherichia coli, e.g. as shown in the respective line in column 5 of Table I, application no. 4, has been published in B. And the activity of the gene product thereof is the activity of dihydroxyacid dehydratase.

Accordingly, in one embodiment, the process of the present invention for producing threonine in a non-human organism, like a microorganism or a plant or a part thereof, comprises increasing or generating the activity of a gene product with the activity of a gene product conferring the activity of “dihydroxyacid dehydratase”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B3771, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B3771, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B3771, or a functional equivalent or a homolog thereof as depicted in column 8 of Table II, application no. 4, preferably a homolog or functional equivalent as depicted in column 8 of Table II B, application no. 4, and being depicted in the same respective line as said B3771, and preferably the activity is increased non-targeted.

Accordingly, in one embodiment, the molecule which activity is to be increased in the process of the invention is the gene product with an activity as a “dihydroxyacid dehydratase”, preferably it is the molecule of section (a) or (b) of this paragraph.

In particular, it was observed that in plants, especially in Arabidopsis thaliana, increasing or generating the activity of a gene product non-targeted with the activity of a “dihydroxyacid dehydratase”, preferably being encoded by a gene comprising the nucleic acid sequence SEQ ID NO. 10252, preferably the coding region thereof, conferred the production of or the increase in threonine compared with the wild type control.

The nucleic acid sequence of B3774 from Escherichia coli, e.g. as shown in the respective line in column 5 of Table I, application no. 4, has been published in B. And the activity of the gene product thereof is the activity of ketol-acid reductoisomerase.

Accordingly, in one embodiment, the process of the present invention for producing threonine in a non-human organism, like a microorganism or a plant or a part thereof, comprises increasing or generating the activity of a gene product with the activity of a gene product conferring the activity of “ketol-acid reductoisomerase”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B3774, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B3774, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B3774, or a functional equivalent or a homolog thereof as depicted in column 8 of Table II, application no. 4, preferably a homolog or functional equivalent as depicted in column 8 of Table II B, application no. 4, and being depicted in the same respective line as said B3774, and preferably the activity is increased non-targeted.

Accordingly, in one embodiment, the molecule which activity is to be increased in the process of the invention is the gene product with an activity as a “ketol-acid reductoisomerase”, preferably it is the molecule of section (a) or (b) of this paragraph.

In particular, it was observed that in plants, especially in Arabidopsis thaliana, increasing or generating the activity of a gene product non-targeted with the activity of a “ketol-acid reductoisomerase”, preferably being encoded by a gene comprising the nucleic acid sequence SEQ ID NO. 94386, preferably the coding region thereof, conferred the production of or the increase in threonine compared with the wild type control.

The nucleic acid sequence of B3793 from Escherichia coli, e.g. as shown in the respective line in column 5 of Table I, application no. 4, has been published in B. And the activity of the gene product thereof is the activity of enterobacterial common antigen polymerase.

Accordingly, in one embodiment, the process of the present invention for producing threonine in a non-human organism, like a microorganism or a plant or a part thereof, comprises increasing or generating the activity of a gene product with the activity of a gene product conferring the activity of “enterobacterial common antigen polymerase”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B3793, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B3793, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B3793, or a functional equivalent or a homolog thereof as depicted in column 8 of Table II, application no. 4, preferably a homolog or functional equivalent as depicted in column 8 of Table II B, application no. 4, and being depicted in the same respective line as said B3793, and preferably the activity is increased non-targeted.

Accordingly, in one embodiment, the molecule which activity is to be increased in the process of the invention is the gene product with an activity as a “enterobacterial common antigen polymerase”, preferably it is the molecule of section (a) or (b) of this paragraph.

In particular, it was observed that in plants, especially in Arabidopsis thaliana, increasing or generating the activity of a gene product non-targeted with the activity of a “enterobacterial common antigen polymerase”, preferably being encoded by a gene comprising the nucleic acid sequence SEQ ID NO. 43800, preferably the coding region thereof, conferred the production of or the increase in threonine compared with the wild type control.

The nucleic acid sequence of B3813 from Escherichia coli, e.g. as shown in the respective line in column 5 of Table I, application no. 4, has been published in B. And the activity of the gene product thereof is the activity of DNA helicase II.

Accordingly, in one embodiment, the process of the present invention for producing threonine in a non-human organism, like a microorganism or a plant or a part thereof, comprises increasing or generating the activity of a gene product with the activity of a gene product conferring the activity of “DNA helicase II”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B3813, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B3813, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B3813, or a functional equivalent or a homolog thereof as depicted in column 8 of Table II, application no. 4, preferably a homolog or functional equivalent as depicted in column 8 of Table II B, application no. 4, and being depicted in the same respective line as said B3813, and preferably the activity is increased non-targeted.

Accordingly, in one embodiment, the molecule which activity is to be increased in the process of the invention is the gene product with an activity as a “DNA helicase II”, preferably it is the molecule of section (a) or (b) of this paragraph.

In particular, it was observed that in plants, especially in Arabidopsis thaliana, increasing or generating the activity of a gene product non-targeted with the activity of a “DNA helicase II”, preferably being encoded by a gene comprising the nucleic acid sequence SEQ ID NO. 43839, preferably the coding region thereof, conferred the production of or the increase in threonine compared with the wild type control.

The nucleic acid sequence of B3872 from Escherichia coli, e.g. as shown in the respective line in column 5 of Table I, application no. 4, has been published in B. And the activity of the gene product thereof is the activity of transcriptional regulator.

Accordingly, in one embodiment, the process of the present invention for producing threonine in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B3872, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B3872, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B3872, or a functional equivalent or a homolog thereof as depicted in column 8 of Table II, application no. 4, preferably a homolog or functional equivalent as depicted in column 8 of Table II B, application no. 4, and being depicted in the same respective line as said B3872, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 83821, preferably the coding region thereof, conferred the production of or the increase in threonine compared with the wild type control.

The nucleic acid sequence of B3989 from Escherichia coli, e.g. as shown in the respective line in column 5 of Table I, application no. 4, has been published in B. And the activity of the gene product thereof is the activity of b3989-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “b3989-protein”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B3989, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B3989, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B3989, or a functional equivalent or a homolog thereof as depicted in column 8 of Table II, application no. 4, preferably a homolog or functional equivalent as depicted in column 8 of Table II B, application no. 4, and being depicted in the same respective line as said B3989, and preferably the activity is increased non-targeted.

Accordingly, in one embodiment, the molecule which activity is to be increased in the process of the invention is the gene product with an activity as a “b3989-protein”, preferably it is the molecule of section (a) or (b) of this paragraph.

In particular, it was observed that in plants, especially in Arabidopsis thaliana, increasing or generating the activity of a gene product non-targeted with the activity of a “b3989-protein”, preferably being encoded by a gene comprising the nucleic acid sequence SEQ ID NO. 44372, preferably the coding region thereof, conferred the production of or the increase in threonine compared with the wild type control.

The nucleic acid sequence of B4024 from Escherichia coli, e.g. as shown in the respective line in column 5 of Table I, application no. 4, has been published in B. And the activity of the gene product thereof is the activity of aspartate kinase.

Accordingly, in one embodiment, the process of the present invention for producing threonine in a non-human organism, like a microorganism or a plant or a part thereof, comprises increasing or generating the activity of a gene product with the activity of a gene product conferring the activity of “aspartate kinase”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B4024, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B4024, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B4024, or a functional equivalent or a homolog thereof as depicted in column 8 of Table II, application no. 4, preferably a homolog or functional equivalent as depicted in column 8 of Table II B, application no. 4, and being depicted in the same respective line as said B4024, and preferably the activity is increased plastidic.

Accordingly, in one embodiment, the molecule which activity is to be increased in the process of the invention is the gene product with an activity as a “aspartate kinase”, preferably it is the molecule of section (a) or (b) of this paragraph.

In particular, it was observed that in plants, especially in Arabidopsis thaliana, increasing or generating the activity of a gene product plastidic with the activity of a “aspartate kinase”, preferably being encoded by a gene comprising the nucleic acid sequence SEQ ID NO. 83835, preferably the coding region thereof, conferred the production of or the increase in threonine compared with the wild type control.

The nucleic acid sequence of B4029 from Escherichia coli, e.g. as shown in the respective line in column 5 of Table I, application no. 4, has been published in B. And the activity of the gene product thereof is the activity of b4029-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “b4029-protein”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B4029, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B4029, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B4029, or a functional equivalent or a homolog thereof as depicted in column 8 of Table II, application no. 4, preferably a homolog or functional equivalent as depicted in column 8 of Table II B, application no. 4, and being depicted in the same respective line as said B4029, and preferably the activity is increased non-targeted.

Accordingly, in one embodiment, the molecule which activity is to be increased in the process of the invention is the gene product with an activity as a “b4029-protein”, preferably it is the molecule of section (a) or (b) of this paragraph.

In particular, it was observed that in plants, especially in Arabidopsis thaliana, increasing or generating the activity of a gene product non-targeted with the activity of a “b4029-protein”, preferably being encoded by a gene comprising the nucleic acid sequence SEQ ID NO. 10740, preferably the coding region thereof, conferred the production of or the increase in threonine compared with the wild type control.

The nucleic acid sequence of B4056 from Escherichia coli, e.g. as shown in the respective line in column 5 of Table I, application no. 4, has been published in B. And the activity of the gene product thereof is the activity of b4056-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “b4056-protein”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B4056, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B4056, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B4056, or a functional equivalent or a homolog thereof as depicted in column 8 of Table II, application no. 4, preferably a homolog or functional equivalent as depicted in column 8 of Table II B, application no. 4, and being depicted in the same respective line as said B4056, and preferably the activity is increased non-targeted.

Accordingly, in one embodiment, the molecule which activity is to be increased in the process of the invention is the gene product with an activity as a “b4056-protein”, preferably it is the molecule of section (a) or (b) of this paragraph.

In particular, it was observed that in plants, especially in Arabidopsis thaliana, increasing or generating the activity of a gene product non-targeted with the activity of a “b4056-protein”, preferably being encoded by a gene comprising the nucleic acid sequence SEQ ID NO. 94542, preferably the coding region thereof, conferred the production of or the increase in threonine compared with the wild type control.

The nucleic acid sequence of B4250 from Escherichia coli, e.g. as shown in the respective line in column 5 of Table I, application no. 4, has been published in B. And the activity of the gene product thereof is the activity of b4250-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “b4250-protein”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B4250, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B4250, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B4250, or a functional equivalent or a homolog thereof as depicted in column 8 of Table II, application no. 4, preferably a homolog or functional equivalent as depicted in column 8 of Table II B, application no. 4, and being depicted in the same respective line as said B4250, and preferably the activity is increased non-targeted.

Accordingly, in one embodiment, the molecule which activity is to be increased in the process of the invention is the gene product with an activity as a “b4250-protein”, preferably it is the molecule of section (a) or (b) of this paragraph.

In particular, it was observed that in plants, especially in Arabidopsis thaliana, increasing or generating the activity of a gene product non-targeted with the activity of a “b4250-protein”, preferably being encoded by a gene comprising the nucleic acid sequence SEQ ID NO. 94735, preferably the coding region thereof, conferred the production of or the increase in threonine compared with the wild type control.

The nucleic acid sequence of B4256 from Escherichia coli, e.g. as shown in the respective line in column 5 of Table I, application no. 4, has been published in B. And the activity of the gene product thereof is the activity of acetyltransferase.

Accordingly, in one embodiment, the process of the present invention for producing threonine in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said B4256, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said B4256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said B4256, or a functional equivalent or a homolog thereof as depicted in column 8 of Table II, application no. 4, preferably a homolog or functional equivalent as depicted in column 8 of Table II B, application no. 4, and being depicted in the same respective line as said B4256, and preferably the activity is increased non-targeted.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 45321, preferably the coding region thereof, conferred the production of or the increase in threonine compared with the wild type control.

The nucleic acid sequence of GM02LC12622 from Glycine max, e.g. as shown in the respective line in column 5 of Table I, application no. 4, is unpublished. And the activity of the gene product thereof is the activity of glutaredoxin.

Accordingly, in one embodiment, the process of the present invention for producing threonine in a non-human organism, like a microorganism or a plant or a part thereof, comprises increasing or generating the activity of a gene product with the activity of a gene product conferring the activity of “glutaredoxin”, especially from Glycine max or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said GM02LC12622, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said GM02LC12622, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said GM02LC12622, or a functional equivalent or a homolog thereof as depicted in column 8 of Table II, application no. 4, preferably a homolog or functional equivalent as depicted in column 8 of Table II B, application no. 4, and being depicted in the same respective line as said GM02LC12622, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 10811, preferably the coding region thereof, conferred the production of or the increase in threonine compared with the wild type control.

The nucleic acid sequence of GM02LC21368 from Glycine max, e.g. as shown in the respective line in column 5 of Table I, application no. 4, is unpublished. And the activity of the gene product thereof is the activity of transcription factor.

Accordingly, in one embodiment, the process of the present invention for producing threonine in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said GM02LC21368, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said GM02LC21368, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said GM02LC21368, or a functional equivalent or a homolog thereof as depicted in column 8 of Table II, application no. 4, preferably a homolog or functional equivalent as depicted in column 8 of Table II B, application no. 4, and being depicted in the same respective line as said GM02LC21368, and preferably the activity is increased non-targeted.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 84198, preferably the coding region thereof, conferred the production of or the increase in threonine compared with the wild type control.

The nucleic acid sequence of GM02LC44512 from Glycine max, e.g. as shown in the respective line in column 5 of Table I, application no. 4, is unpublished. And the activity of the gene product thereof is the activity of cyclin D.

Accordingly, in one embodiment, the process of the present invention for producing threonine in a non-human organism, like a microorganism or a plant or a part thereof, comprises increasing or generating the activity of a gene product with the activity of a gene product conferring the activity of “cyclin D”, especially from Glycine max or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said GM02LC44512, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said GM02LC44512, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said GM02LC44512, or a functional equivalent or a homolog thereof as depicted in column 8 of Table II, application no. 4, preferably a homolog or functional equivalent as depicted in column 8 of Table II B, application no. 4, and being depicted in the same respective line as said GM02LC44512, and preferably the activity is increased non-targeted.

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 46850, preferably the coding region thereof, conferred the production of or the increase in threonine compared with the wild type control.

The nucleic acid sequence of Sll0170 from Synechocystis sp., e.g. as shown in the respective line in column 5 of Table I, application no. 4, has been published in C. And the activity of the gene product thereof is the activity of heat shock protein.

Accordingly, in one embodiment, the process of the present invention for producing threonine in a non-human organism, like a microorganism or a plant or a part thereof, comprises increasing or generating the activity of a gene product with the activity of a gene product conferring the activity of “heat shock protein”, especially from Synechocystis sp. or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Sll0170, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Sll0170, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Sll0170, or a functional equivalent or a homolog thereof as depicted in column 8 of Table II, application no. 4, preferably a homolog or functional equivalent as depicted in column 8 of Table II B, application no. 4, and being depicted in the same respective line as said Sll0170, and preferably the activity is increased non-targeted.

Accordingly, in one embodiment, the molecule which activity is to be increased in the process of the invention is the gene product with an activity as a “heat shock protein”, preferably it is the molecule of section (a) or (b) of this paragraph.

In particular, it was observed that in plants, especially in Arabidopsis thaliana, increasing or generating the activity of a gene product non-targeted with the activity of a “heat shock protein”, preferably being encoded by a gene comprising the nucleic acid sequence SEQ ID NO. 48138, preferably the coding region thereof, conferred the production of or the increase in threonine compared with the wild type control.

The nucleic acid sequence of 5110290 from Synechocystis sp., e.g. as shown in the respective line in column 5 of Table I, application no. 4, has been published in C. And the activity of the gene product thereof is the activity of polyphosphate kinase.

Accordingly, in one embodiment, the process of the present invention for producing threonine in a non-human organism, like a microorganism or a plant or a part thereof, comprises increasing or generating the activity of a gene product with the activity of a gene product conferring the activity of “polyphosphate kinase”, especially from Synechocystis sp. or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Sll0290, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Sll0290, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Sll0290, or a functional equivalent or a homolog thereof as depicted in column 8 of Table II, application no. 4, preferably a homolog or functional equivalent as depicted in column 8 of Table II B, application no. 4, and being depicted in the same respective line as said Sll0290, and preferably the activity is increased non-targeted.

Accordingly, in one embodiment, the molecule which activity is to be increased in the process of the invention is the gene product with an activity as a “polyphosphate kinase”, preferably it is the molecule of section (a) or (b) of this paragraph.

In particular, it was observed that in plants, especially in Arabidopsis thaliana, increasing or generating the activity of a gene product non-targeted with the activity of a “polyphosphate kinase”, preferably being encoded by a gene comprising the nucleic acid sequence SEQ ID NO. 49828, preferably the coding region thereof, conferred the production of or the increase in threonine compared with the wild type control.

The nucleic acid sequence of Sll0374 from Synechocystis sp., e.g. as shown in the respective line in column 5 of Table I, application no. 4, has been published in C. And the activity of the gene product thereof is the activity of branched chain amino acid ABC transporter ATP-binding protein

Accordingly, in one embodiment, the process of the present invention for producing threonine in a non-human organism, like a microorganism or a plant or a part thereof, comprises increasing or generating the activity of a gene product with the activity of a gene product conferring the activity of “branched chain amino acid ABC transporter ATP-binding protein”, especially from Synechocystis sp. or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Sll0374, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Sll0374, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Sll0374, or a functional equivalent or a homolog thereof as depicted in column 8 of Table II, application no. 4, preferably a homolog or functional equivalent as depicted in column 8 of Table II B, application no. 4, and being depicted in the same respective line as said Sll0374, and preferably the activity is increased plastidic.

Accordingly, in one embodiment, the molecule which activity is to be increased in the process of the invention is the gene product with an activity as a “branched chain amino acid ABC transporter ATP-binding protein”, preferably it is the molecule of section (a) or (b) of this paragraph.

In particular, it was observed that in plants, especially in Arabidopsis thaliana, increasing or generating the activity of a gene product plastidic with the activity of a “branched chain amino acid ABC transporter ATP-binding protein”, preferably being encoded by a gene comprising the nucleic acid sequence SEQ ID NO. 94741, preferably the coding region thereof, conferred the production of or the increase in threonine compared with the wild type control.

The nucleic acid sequence of Sll0378 from Synechocystis sp., e.g. as shown in the respective line in column 5 of Table I, application no. 4, has been published in C. And the activity of the gene product thereof is the activity of uroporphyrin-III C-methyltransferase.

Accordingly, in one embodiment, the process of the present invention for producing threonine in a non-human organism, like a microorganism or a plant or a part thereof, comprises increasing or generating the activity of a gene product with the activity of a gene product conferring the activity of “uroporphyrin-III C-methyltransferase”, especially from Synechocystis sp. or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Sll0378, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Sll0378, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Sll0378, or a functional equivalent or a homolog thereof as depicted in column 8 of Table II, application no. 4, preferably a homolog or functional equivalent as depicted in column 8 of Table II B, application no. 4, and being depicted in the same respective line as said Sll0378, and preferably the activity is increased plastidic.

Accordingly, in one embodiment, the molecule which activity is to be increased in the process of the invention is the gene product with an activity as a “uroporphyrin-III C-methyltransferase”, preferably it is the molecule of section (a) or (b) of this paragraph.

In particular, it was observed that in plants, especially in Arabidopsis thaliana, increasing or generating the activity of a gene product plastidic with the activity of a “uroporphyrin-III C-methyltransferase”, preferably being encoded by a gene comprising the nucleic acid sequence SEQ ID NO. 84869, preferably the coding region thereof, conferred the production of or the increase in threonine compared with the wild type control.

The nucleic acid sequence of Sll0418 from Synechocystis sp., e.g. as shown in the respective line in column 5 of Table I, application no. 4, has been published in C. And the activity of the gene product thereof is the activity of sll0418-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “sll0418-protein”, especially from Synechocystis sp. or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Sll0418, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Sll0418, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Sll0418, or a functional equivalent or a homolog thereof as depicted in column 8 of Table II, application no. 4, preferably a homolog or functional equivalent as depicted in column 8 of Table II B, application no. 4, and being depicted in the same respective line as said Sll0418, and preferably the activity is increased non-targeted.

Accordingly, in one embodiment, the molecule which activity is to be increased in the process of the invention is the gene product with an activity as a “sll0418-protein”, preferably it is the molecule of section (a) or (b) of this paragraph.

In particular, it was observed that in plants, especially in Arabidopsis thaliana, increasing or generating the activity of a gene product non-targeted with the activity of a “sll0418-protein”, preferably being encoded by a gene comprising the nucleic acid sequence SEQ ID NO. 94942, preferably the coding region thereof, conferred the production of or the increase in threonine compared with the wild type control.

The nucleic acid sequence of Sll0816 from Synechocystis sp., e.g. as shown in the respective line in column 5 of Table I, application no. 4, has been published in C. And the activity of the gene product thereof is the activity of oxireductase.

Accordingly, in one embodiment, the process of the present invention for producing threonine in a non-human organism, like a microorganism or a plant or a part thereof, comprises increasing or generating the activity of a gene product with the activity of a gene product conferring the activity of “oxireductase”, especially from Synechocystis sp. or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said 5110816, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Sll0816, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Sll0816, or a functional equivalent or a homolog thereof as depicted in column 8 of Table II, application no. 4, preferably a homolog or functional equivalent as depicted in column 8 of Table II B, application no. 4, and being depicted in the same respective line as said Sll0816, and preferably the activity is increased non-targeted.

Accordingly, in one embodiment, the molecule which activity is to be increased in the process of the invention is the gene product with an activity as a “oxireductase”, preferably it is the molecule of section (a) or (b) of this paragraph.

In particular, it was observed that in plants, especially in Arabidopsis thaliana, increasing or generating the activity of a gene product non-targeted with the activity of a “oxireductase”, preferably being encoded by a gene comprising the nucleic acid sequence SEQ ID NO. 51198, preferably the coding region thereof, conferred the production of or the increase in threonine compared with the wild type control.

The nucleic acid sequence of Sll0891 from Synechocystis sp., e.g. as shown in the respective line in column 5 of Table I, application no. 4, has been published in C. And the activity of the gene product thereof is the activity of malate dehydrogenase.

Accordingly, in one embodiment, the process of the present invention for producing threonine in a non-human organism, like a microorganism or a plant or a part thereof, comprises increasing or generating the activity of a gene product with the activity of a gene product conferring the activity of “malate dehydrogenase”, especially from Synechocystis sp. or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Sll0891, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Sll0891, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Sll0891, or a functional equivalent or a homolog thereof as depicted in column 8 of Table II, application no. 4, preferably a homolog or functional equivalent as depicted in column 8 of Table II B, application no. 4, and being depicted in the same respective line as said Sll0891, and preferably the activity is increased plastidic.

Accordingly, in one embodiment, the molecule which activity is to be increased in the process of the invention is the gene product with an activity as a “malate dehydrogenase”, preferably it is the molecule of section (a) or (b) of this paragraph.

In particular, it was observed that in plants, especially in Arabidopsis thaliana, increasing or generating the activity of a gene product plastidic with the activity of a “malate dehydrogenase”, preferably being encoded by a gene comprising the nucleic acid sequence SEQ ID NO. 51268, preferably the coding region thereof, conferred the production of or the increase in threonine compared with the wild type control.

The nucleic acid sequence of Sll0901 from Synechocystis sp., e.g. as shown in the respective line in column 5 of Table I, application no. 4, has been published in C. And the activity of the gene product thereof is the activity of phosphoribosylaminoimidazole carboxylase catalytic subunit.

Accordingly, in one embodiment, the process of the present invention for producing threonine in a non-human organism, like a microorganism or a plant or a part thereof, comprises increasing or generating the activity of a gene product with the activity of a gene product conferring the activity of “phosphoribosylaminoimidazole carboxylase catalytic subunit”, especially from Synechocystis sp. or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Sll0901, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Sll0901, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Sll0901, or a functional equivalent or a homolog thereof as depicted in column 8 of Table II, application no. 4, preferably a homolog or functional equivalent as depicted in column 8 of Table II B, application no. 4, and being depicted in the same respective line as said Sll0901, and preferably the activity is increased mitochondrial.

Accordingly, in one embodiment, the molecule which activity is to be increased in the process of the invention is the gene product with an activity as a “phosphoribosylaminoimidazole carboxylase catalytic subunit”, preferably it is the molecule of section (a) or (b) of this paragraph.

In particular, it was observed that in plants, especially in Arabidopsis thaliana, increasing or generating the activity of a gene product mitochondrial with the activity of a “phosphoribosylaminoimidazole carboxylase catalytic subunit”, preferably being encoded by a gene comprising the nucleic acid sequence SEQ ID NO. 51632, preferably the coding region thereof, conferred the production of or the increase in threonine compared with the wild type control.

The nucleic acid sequence of Sll0945 from Synechocystis sp., e.g. as shown in the respective line in column 5 of Table I, application no. 4, has been published in C. And the activity of the gene product thereof is the activity of glycogen synthase.

Accordingly, in one embodiment, the process of the present invention for producing threonine in a non-human organism, like a microorganism or a plant or a part thereof, comprises increasing or generating the activity of a gene product with the activity of a gene product conferring the activity of “glycogen synthase”, especially from Synechocystis sp. or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Sll0945, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Sll0945, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Sll0945, or a functional equivalent or a homolog thereof as depicted in column 8 of Table II, application no. 4, preferably a homolog or functional equivalent as depicted in column 8 of Table II B, application no. 4, and being depicted in the same respective line as said Sll0945, and preferably the activity is increased non-targeted.

Accordingly, in one embodiment, the molecule which activity is to be increased in the process of the invention is the gene product with an activity as a “glycogen synthase”, preferably it is the molecule of section (a) or (b) of this paragraph.

In particular, it was observed that in plants, especially in Arabidopsis thaliana, increasing or generating the activity of a gene product non-targeted with the activity of a “glycogen synthase”, preferably being encoded by a gene comprising the nucleic acid sequence SEQ ID NO. 52364, preferably the coding region thereof, conferred the production of or the increase in threonine compared with the wild type control.

The nucleic acid sequence of Sll1393 from Synechocystis sp., e.g. as shown in the respective line in column 5 of Table I, application no. 4, has been published in C. And the activity of the gene product thereof is the activity of glycogen (starch) synthase.

Accordingly, in one embodiment, the process of the present invention for producing threonine in a non-human organism, like a microorganism or a plant or a part thereof, comprises increasing or generating the activity of a gene product with the activity of a gene product conferring the activity of “glycogen (starch) synthase”, especially from Synechocystis sp. or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Sll1393, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Sll1393, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Sll1393, or a functional equivalent or a homolog thereof as depicted in column 8 of Table II, application no. 4, preferably a homolog or functional equivalent as depicted in column 8 of Table II B, application no. 4, and being depicted in the same respective line as said Sll1393, and preferably the activity is increased non-targeted.

Accordingly, in one embodiment, the molecule which activity is to be increased in the process of the invention is the gene product with an activity as a “glycogen (starch) synthase”, preferably it is the molecule of section (a) or (b) of this paragraph.

In particular, it was observed that in plants, especially in Arabidopsis thaliana, increasing or generating the activity of a gene product non-targeted with the activity of a “glycogen (starch) synthase”, preferably being encoded by a gene comprising the nucleic acid sequence SEQ ID NO. 53456, preferably the coding region thereof, conferred the production of or the increase in threonine compared with the wild type control.

The nucleic acid sequence of Sll1450 from Synechocystis sp., e.g. as shown in the respective line in column 5 of Table I, application no. 4, has been published in C. And the activity of the gene product thereof is the activity of nitrate/nitrite transport protein.

Accordingly, in one embodiment, the process of the present invention for producing threonine in a non-human organism, like a microorganism or a plant or a part thereof, comprises increasing or generating the activity of a gene product with the activity of a gene product conferring the activity of “nitrate/nitrite transport protein”, especially from Synechocystis sp. or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Sll1450, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Sll1450, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Sll1450, or a functional equivalent or a homolog thereof as depicted in column 8 of Table II, application no. 4, preferably a homolog or functional equivalent as depicted in column 8 of Table II B, application no. 4, and being depicted in the same respective line as said Sll1450, and preferably the activity is increased mitochondrial.

Accordingly, in one embodiment, the molecule which activity is to be increased in the process of the invention is the gene product with an activity as a “nitrate/nitrite transport protein”, preferably it is the molecule of section (a) or (b) of this paragraph.

In particular, it was observed that in plants, especially in Arabidopsis thaliana, increasing or generating the activity of a gene product mitochondrial with the activity of a “nitrate/nitrite transport protein”, preferably being encoded by a gene comprising the nucleic acid sequence SEQ ID NO. 54337, preferably the coding region thereof, conferred the production of or the increase in threonine compared with the wild type control.

The nucleic acid sequence of Sll1498 from Synechocystis sp., e.g. as shown in the respective line in column 5 of Table I, application no. 4, has been published in C. And the activity of the gene product thereof is the activity of carbamoyl-phosphate synthase subunit.

Accordingly, in one embodiment, the process of the present invention for producing threonine in a non-human organism, like a microorganism or a plant or a part thereof, comprises increasing or generating the activity of a gene product with the activity of a gene product conferring the activity of “carbamoyl-phosphate synthase subunit”, especially from Synechocystis sp. or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Sll1498, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Sll1498, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Sll1498, or a functional equivalent or a homolog thereof as depicted in column 8 of Table II, application no. 4, preferably a homolog or functional equivalent as depicted in column 8 of Table II B, application no. 4, and being depicted in the same respective line as said Sll1498, and preferably the activity is increased plastidic.

Accordingly, in one embodiment, the molecule which activity is to be increased in the process of the invention is the gene product with an activity as a “carbamoyl-phosphate synthase subunit”, preferably it is the molecule of section (a) or (b) of this paragraph.

In particular, it was observed that in plants, especially in Arabidopsis thaliana, increasing or generating the activity of a gene product plastidic with the activity of a “carbamoyl-phosphate synthase subunit”, preferably being encoded by a gene comprising the nucleic acid sequence SEQ ID NO. 95047, preferably the coding region thereof, conferred the production of or the increase in threonine compared with the wild type control.

The nucleic acid sequence of Sll1545 from Synechocystis sp., e.g. as shown in the respective line in column 5 of Table I, application no. 4, has been published in C. And the activity of the gene product thereof is the activity of glutathione S-transferase.

Accordingly, in one embodiment, the process of the present invention for producing threonine in a non-human organism, like a microorganism or a plant or a part thereof, comprises increasing or generating the activity of a gene product with the activity of a gene product conferring the activity of “glutathione 5-transferase”, especially from Synechocystis sp. or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Sll1545, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Sll1545, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Sll1545, or a functional equivalent or a homolog thereof as depicted in column 8 of Table II, application no. 4, preferably a homolog or functional equivalent as depicted in column 8 of Table II B, application no. 4, and being depicted in the same respective line as said Sll1545, and preferably the activity is increased mitochondrial.

Accordingly, in one embodiment, the molecule which activity is to be increased in the process of the invention is the gene product with an activity as a “glutathione S-transferase”, preferably it is the molecule of section (a) or (b) of this paragraph.

In particular, it was observed that in plants, especially in Arabidopsis thaliana, increasing or generating the activity of a gene product mitochondrial with the activity of a “glutathione S-transferase”, preferably being encoded by a gene comprising the nucleic acid sequence SEQ ID NO. 11423, preferably the coding region thereof, conferred the production of or the increase in threonine compared with the wild type control.

The nucleic acid sequence of Sll1598 from Synechocystis sp., e.g. as shown in the respective line in column 5 of Table I, application no. 4, has been published in C. And the activity of the gene product thereof is the activity of ABC metal ion transporter substrate-binding protein.

Accordingly, in one embodiment, the process of the present invention for producing threonine in a non-human organism, like a microorganism or a plant or a part thereof, comprises increasing or generating the activity of a gene product with the activity of a gene product conferring the activity of “ABC metal ion transporter substrate-binding protein”, especially from Synechocystis sp. or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Sll1598, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Sll1598, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Sll1598, or a functional equivalent or a homolog thereof as depicted in column 8 of Table II, application no. 4, preferably a homolog or functional equivalent as depicted in column 8 of Table II B, application no. 4, and being depicted in the same respective line as said Sll1598, and preferably the activity is increased mitochondrial.

Accordingly, in one embodiment, the molecule which activity is to be increased in the process of the invention is the gene product with an activity as a “ABC metal ion transporter substratebinding protein”, preferably it is the molecule of section (a) or (b) of this paragraph.

In particular, it was observed that in plants, especially in Arabidopsis thaliana, increasing or generating the activity of a gene product mitochondrial with the activity of a “ABC metal ion transporter substrate-binding protein”, preferably being encoded by a gene comprising the nucleic acid sequence SEQ ID NO. 85743, preferably the coding region thereof, conferred the production of or the increase in threonine compared with the wild type control.

The nucleic acid sequence of Sll1917 from Synechocystis sp., e.g. as shown in the respective line in column 5 of Table I, application no. 4, has been published in C. And the activity of the gene product thereof is the activity of coproporphyrinogen III oxidase.

Accordingly, in one embodiment, the process of the present invention for producing threonine in a non-human organism, like a microorganism or a plant or a part thereof, comprises increasing or generating the activity of a gene product with the activity of a gene product conferring the activity of “coproporphyrinogen III oxidase”, especially from Synechocystis sp. or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Sll1917, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Sll1917, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Sll1917, or a functional equivalent or a homolog thereof as depicted in column 8 of Table II, application no. 4, preferably a homolog or functional equivalent as depicted in column 8 of Table II B, application no. 4, and being depicted in the same respective line as said Sll1917, and preferably the activity is increased mitochondrial.

Accordingly, in one embodiment, the molecule which activity is to be increased in the process of the invention is the gene product with an activity as a “coproporphyrinogen III oxidase”, preferably it is the molecule of section (a) or (b) of this paragraph.

In particular, it was observed that in plants, especially in Arabidopsis thaliana, increasing or generating the activity of a gene product mitochondrial with the activity of a “coproporphyrinogen III oxidase”, preferably being encoded by a gene comprising the nucleic acid sequence SEQ ID NO. 11471, preferably the coding region thereof, conferred the production of or the increase in threonine compared with the wild type control.

The nucleic acid sequence of Slr0150 from Synechocystis sp., e.g. as shown in the respective line in column 5 of Table I, application no. 4, has been published in C. And the activity of the gene product thereof is the activity of ferredoxin.

Accordingly, in one embodiment, the process of the present invention for producing threonine in a non-human organism, like a microorganism or a plant or a part thereof, comprises increasing or generating the activity of a gene product with the activity of a gene product conferring the activity of “ferredoxin”, especially from Synechocystis sp. or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Slr0150, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Slr0150, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Slr0150, or a functional equivalent or a homolog thereof as depicted in column 8 of Table II, application no. 4, preferably a homolog or functional equivalent as depicted in column 8 of Table II B, application no. 4, and being depicted in the same respective line as said Slr0150, and preferably the activity is increased mitochondrial.

Accordingly, in one embodiment, the molecule which activity is to be increased in the process of the invention is the gene product with an activity as a “ferredoxin”, preferably it is the molecule of section (a) or (b) of this paragraph.

In particular, it was observed that in plants, especially in Arabidopsis thaliana, increasing or generating the activity of a gene product mitochondrial with the activity of a “ferredoxin”, preferably being encoded by a gene comprising the nucleic acid sequence SEQ ID NO. 95527, preferably the coding region thereof, conferred the production of or the increase in threonine compared with the wild type control.

The nucleic acid sequence of Slr0383 from Synechocystis sp., e.g. as shown in the respective line in column 5 of Table I, application no. 4, has been published in C. And the activity of the gene product thereof is the activity of slr0383-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “slr0383-protein”, especially from Synechocystis sp. or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Slr0383, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Slr0383, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Slr0383, or a functional equivalent or a homolog thereof as depicted in column 8 of Table II, application no. 4, preferably a homolog or functional equivalent as depicted in column 8 of Table II B, application no. 4, and being depicted in the same respective line as said Slr0383, and preferably the activity is increased non-targeted.

Accordingly, in one embodiment, the molecule which activity is to be increased in the process of the invention is the gene product with an activity as a “slr0383-protein”, preferably it is the molecule of section (a) or (b) of this paragraph.

In particular, it was observed that in plants, especially in Arabidopsis thaliana, increasing or generating the activity of a gene product non-targeted with the activity of a “slr0383-protein”, preferably being encoded by a gene comprising the nucleic acid sequence SEQ ID NO. 95744, preferably the coding region thereof, conferred the production of or the increase in threonine compared with the wild type control.

The nucleic acid sequence of Slr0597 from Synechocystis sp., e.g. as shown in the respective line in column 5 of Table I, application no. 4, has been published in C. And the activity of the gene product thereof is the activity of bifunctional purine biosynthesis protein.

Accordingly, in one embodiment, the process of the present invention for producing threonine in a non-human organism, like a microorganism or a plant or a part thereof, comprises increasing or generating the activity of a gene product with the activity of a gene product conferring the activity of “bifunctional purine biosynthesis protein”, especially from Synechocystis sp. or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Slr0597, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Slr0597, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Slr0597, or a functional equivalent or a homolog thereof as depicted in column 8 of Table II, application no. 4, preferably a homolog or functional equivalent as depicted in column 8 of Table II B, application no. 4, and being depicted in the same respective line as said Slr0597, and preferably the activity is increased plastidic.

Accordingly, in one embodiment, the molecule which activity is to be increased in the process of the invention is the gene product with an activity as a “bifunctional purine biosynthesis protein”, preferably it is the molecule of section (a) or (b) of this paragraph.

In particular, it was observed that in plants, especially in Arabidopsis thaliana, increasing or generating the activity of a gene product plastidic with the activity of a “bifunctional purine biosynthesis protein”, preferably being encoded by a gene comprising the nucleic acid sequence SEQ ID NO. 56153, preferably the coding region thereof, conferred the production of or the increase in threonine compared with the wild type control.

The nucleic acid sequence of Slr0657 from Synechocystis sp., e.g. as shown in the respective line in column 5 of Table I, application no. 4, has been published in C. And the activity of the gene product thereof is the activity of aspartate kinase.

Accordingly, in one embodiment, the process of the present invention for producing threonine in a non-human organism, like a microorganism or a plant or a part thereof, comprises increasing or generating the activity of a gene product with the activity of a gene product conferring the activity of “aspartate kinase”, especially from Synechocystis sp. or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Slr0657, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Slr0657, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Slr0657, or a functional equivalent or a homolog thereof as depicted in column 8 of Table II, application no. 4, preferably a homolog or functional equivalent as depicted in column 8 of Table II B, application no. 4, and being depicted in the same respective line as said Slr0657, and preferably the activity is increased mitochondrial.

Accordingly, in one embodiment, the molecule which activity is to be increased in the process of the invention is the gene product with an activity as a “aspartate kinase”, preferably it is the molecule of section (a) or (b) of this paragraph.

In particular, it was observed that in plants, especially in Arabidopsis thaliana, increasing or generating the activity of a gene product mitochondrial with the activity of a “aspartate kinase”, preferably being encoded by a gene comprising the nucleic acid sequence SEQ ID NO. 95857, preferably the coding region thereof, conferred the production of or the increase in threonine compared with the wild type control.

The nucleic acid sequence of Slr0739 from Synechocystis sp., e.g. as shown in the respective line in column 5 of Table I, application no. 4, has been published in C. And the activity of the gene product thereof is the activity of geranylgeranyl pyrophosphate synthase.

Accordingly, in one embodiment, the process of the present invention for producing threonine in a non-human organism, like a microorganism or a plant or a part thereof, comprises increasing or generating the activity of a gene product with the activity of a gene product conferring the activity of “geranylgeranyl pyrophosphate synthase”, especially from Synechocystis sp. or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Slr0739, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Slr0739, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Slr0739, or a functional equivalent or a homolog thereof as depicted in column 8 of Table II, application no. 4, preferably a homolog or functional equivalent as depicted in column 8 of Table II B, application no. 4, and being depicted in the same respective line as said Slr0739, and preferably the activity is increased plastidic.

Accordingly, in one embodiment, the molecule which activity is to be increased in the process of the invention is the gene product with an activity as a “geranylgeranyl pyrophosphate synthase”, preferably it is the molecule of section (a) or (b) of this paragraph.

In particular, it was observed that in plants, especially in Arabidopsis thaliana, increasing or generating the activity of a gene product plastidic with the activity of a “geranylgeranyl pyrophosphate synthase”, preferably being encoded by a gene comprising the nucleic acid sequence SEQ ID NO. 57235, preferably the coding region thereof, conferred the production of or the increase in threonine compared with the wild type control. The nucleic acid sequence of Slr0862 from Synechocystis sp., e.g. as shown in the respective line in column 5 of Table I, application no. 4, has been published in C. And the activity of the gene product thereof is the activity of kinase.

Accordingly, in one embodiment, the process of the present invention for producing threonine in a non-human organism, like a microorganism or a plant or a part thereof, comprises increasing or generating the activity of a gene product with the activity of a gene product conferring the activity of “kinase”, especially from Synechocystis sp. or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Slr0862, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Slr0862, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Slr0862, or a functional equivalent or a homolog thereof as depicted in column 8 of Table II, application no. 4, preferably a homolog or functional equivalent as depicted in column 8 of Table II B, application no. 4, and being depicted in the same respective line as said Slr0862, and preferably the activity is increased mitochondrial.

Accordingly, in one embodiment, the molecule which activity is to be increased in the process of the invention is the gene product with an activity as a “kinase”, preferably it is the molecule of section (a) or (b) of this paragraph.

In particular, it was observed that in plants, especially in Arabidopsis thaliana, increasing or generating the activity of a gene product mitochondrial with the activity of a “kinase”, preferably being encoded by a gene comprising the nucleic acid sequence SEQ ID NO. 87610, preferably the coding region thereof, conferred the production of or the increase in threonine compared with the wild type control.

The nucleic acid sequence of Slr1133 from Synechocystis sp., e.g. as shown in the respective line in column 5 of Table I, application no. 4, has been published in C. And the activity of the gene product thereof is the activity of argininosuccinate lyase.

Accordingly, in one embodiment, the process of the present invention for producing threonine in a non-human organism, like a microorganism or a plant or a part thereof, comprises increasing or generating the activity of a gene product with the activity of a gene product conferring the activity of “argininosuccinate lyase”, especially from Synechocystis sp. or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Slr1133, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Slr1133, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Slr1133, or a functional equivalent or a homolog thereof as depicted in column 8 of Table II, application no. 4, preferably a homolog or functional equivalent as depicted in column 8 of Table II B, application no. 4, and being depicted in the same respective line as said Slr1133, and preferably the activity is increased mitochondrial.

Accordingly, in one embodiment, the molecule which activity is to be increased in the process of the invention is the gene product with an activity as a “argininosuccinate lyase”, preferably it is the molecule of section (a) or (b) of this paragraph.

In particular, it was observed that in plants, especially in Arabidopsis thaliana, increasing or generating the activity of a gene product mitochondrial with the activity of a “argininosuccinate lyase”, preferably being encoded by a gene comprising the nucleic acid sequence SEQ ID NO. 87853, preferably the coding region thereof, conferred the production of or the increase in threonine compared with the wild type control.

The nucleic acid sequence of Slr1739 from Synechocystis sp., e.g. as shown in the respective line in column 5 of Table I, application no. 4, has been published in C. And the activity of the gene product thereof is the activity of photosystem II protein.

Accordingly, in one embodiment, the process of the present invention for producing threonine in a non-human organism, like a microorganism or a plant or a part thereof, comprises increasing or generating the activity of a gene product with the activity of a gene product conferring the activity of “photosystem II protein”, especially from Synechocystis sp. or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Slr1739, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Slr1739, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Slr1739, or a functional equivalent or a homolog thereof as depicted in column 8 of Table II, application no. 4, preferably a homolog or functional equivalent as depicted in column 8 of Table II B, application no. 4, and being depicted in the same respective line as said Slr1739, and preferably the activity is increased plastidic.

Accordingly, in one embodiment, the molecule which activity is to be increased in the process of the invention is the gene product with an activity as a “photosystem II protein”, preferably it is the molecule of section (a) or (b) of this paragraph.

In particular, it was observed that in plants, especially in Arabidopsis thaliana, increasing or generating the activity of a gene product plastidic with the activity of a “photosystem II protein”, preferably being encoded by a gene comprising the nucleic acid sequence SEQ ID NO. 58731, preferably the coding region thereof, conferred the production of or the increase in threonine compared with the wild type control.

The nucleic acid sequence of TTC0035 from Thermus thermophilus, e.g. as shown in the respective line in column 5 of Table I, application no. 4, has been published in E. And the activity of the gene product thereof is the activity of cell division protein.

Accordingly, in one embodiment, the process of the present invention for producing threonine in a non-human organism, like a microorganism or a plant or a part thereof, comprises increasing or generating the activity of a gene product with the activity of a gene product conferring the activity of “cell division protein”, especially from Thermus thermophilus or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said TTC0035, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said TTC0035, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said TTC0035, or a functional equivalent or a homolog thereof as depicted in column 8 of Table II, application no. 4, preferably a homolog or functional equivalent as depicted in column 8 of Table II B, application no. 4, and being depicted in the same respective line as said TTC0035, and preferably the activity is increased non-targeted.

Accordingly, in one embodiment, the molecule which activity is to be increased in the process of the invention is the gene product with an activity as a “cell division protein”, preferably it is the molecule of section (a) or (b) of this paragraph.

In particular, it was observed that in plants, especially in Arabidopsis thaliana, increasing or generating the activity of a gene product non-targeted with the activity of a “cell division protein”, preferably being encoded by a gene comprising the nucleic acid sequence SEQ ID NO. 60301, preferably the coding region thereof, conferred the production of or the increase in threonine compared with the wild type control.

The nucleic acid sequence of TTC0216 from Thermus thermophilus, e.g. as shown in the respective line in column 5 of Table I, application no. 4, has been published in E. And the activity of the gene product thereof is the activity of branched-chain amino acid ABC transporter permease protein.

Accordingly, in one embodiment, the process of the present invention for producing threonine in a non-human organism, like a microorganism or a plant or a part thereof, comprises increasing or generating the activity of a gene product with the activity of a gene product conferring the activity of “branched-chain amino acid ABC transporter permease protein”, especially from Thermus thermophilus or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said TTC0216, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said TTC0216, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said TTC0216, or a functional equivalent or a homolog thereof as depicted in column 8 of Table II, application no. 4, preferably a homolog or functional equivalent as depicted in column 8 of Table II B, application no. 4, and being depicted in the same respective line as said TTC0216, and preferably the activity is increased non-targeted.

Accordingly, in one embodiment, the molecule which activity is to be increased in the process of the invention is the gene product with an activity as a “branched-chain amino acid ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “branched-chain amino acid ABC transporter permease protein”, preferably being encoded by a gene comprising the nucleic acid sequence SEQ ID NO. 60859, preferably the coding region thereof, conferred the production of or the increase in threonine compared with the wild type control.

The nucleic acid sequence of TTC0881 from Thermus thermophilus, e.g. as shown in the respective line in column 5 of Table I, application no. 4, has been published in E. And the activity of the gene product thereof is the activity of TTC0881-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “TTC0881-protein”, especially from Thermus thermophilus or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said TTC0881, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said TTC0881, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said TTC0881, or a functional equivalent or a homolog thereof as depicted in column 8 of Table II, application no. 4, preferably a homolog or functional equivalent as depicted in column 8 of Table II B, application no. 4, and being depicted in the same respective line as said TTC0881, and preferably the activity is increased non-targeted.

Accordingly, in one embodiment, the molecule which activity is to be increased in the process of the invention is the gene product with an activity as a “TTC0881-protein”, preferably it is the molecule of section (a) or (b) of this paragraph.

In particular, it was observed that in plants, especially in Arabidopsis thaliana, increasing or generating the activity of a gene product non-targeted with the activity of a “TTC0881-protein”, preferably being encoded by a gene comprising the nucleic acid sequence SEQ ID NO. 88875, preferably the coding region thereof, conferred the production of or the increase in threonine compared with the wild type control.

The nucleic acid sequence of TTC0917 from Thermus thermophilus, e.g. as shown in the respective line in column 5 of Table I, application no. 4, has been published in E. And the activity of the gene product thereof is the activity of metal-dependent hydrolase.

Accordingly, in one embodiment, the process of the present invention for producing threonine in a non-human organism, like a microorganism or a plant or a part thereof, comprises increasing or generating the activity of a gene product with the activity of a gene product conferring the activity of “metal-dependent hydrolase”, especially from Thermus thermophilus or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said TTC0917, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said TTC0917, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said TTC0917, or a functional equivalent or a homolog thereof as depicted in column 8 of Table II, application no. 4, preferably a homolog or functional equivalent as depicted in column 8 of Table II B, application no. 4, and being depicted in the same respective line as said TTC0917, and preferably the activity is increased non-targeted.

Accordingly, in one embodiment, the molecule which activity is to be increased in the process of the invention is the gene product with an activity as a “metal-dependent hydrolase”, preferably it is the molecule of section (a) or (b) of this paragraph.

In particular, it was observed that in plants, especially in Arabidopsis thaliana, increasing or generating the activity of a gene product non-targeted with the activity of a “metal-dependent hydrolase”, preferably being encoded by a gene comprising the nucleic acid sequence SEQ ID NO. 61553, preferably the coding region thereof, conferred the production of or the increase in threonine compared with the wild type control.

The nucleic acid sequence of TTC1550 from Thermus thermophilus, e.g. as shown in the respective line in column 5 of Table I, application no. 4, has been published in E. And the activity of the gene product thereof is the activity of homocitrate synthase.

Accordingly, in one embodiment, the process of the present invention for producing threonine in a non-human organism, like a microorganism or a plant or a part thereof, comprises increasing or generating the activity of a gene product with the activity of a gene product conferring the activity of “homocitrate synthase”, especially from Thermus thermophilus or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said TTC1550, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said TTC1550, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said TTC1550, or a functional equivalent or a homolog thereof as depicted in column 8 of Table II, application no. 4, preferably a homolog or functional equivalent as depicted in column 8 of Table II B, application no. 4, and being depicted in the same respective line as said TTC1550, and preferably the activity is increased non-targeted.

Accordingly, in one embodiment, the molecule which activity is to be increased in the process of the invention is the gene product with an activity as a “homocitrate synthase”, preferably it is the molecule of section (a) or (b) of this paragraph.

In particular, it was observed that in plants, especially in Arabidopsis thaliana, increasing or generating the activity of a gene product non-targeted with the activity of a “homocitrate synthase”, preferably being encoded by a gene comprising the nucleic acid sequence SEQ ID NO. 12974, preferably the coding region thereof, conferred the production of or the increase in threonine compared with the wild type control.

The nucleic acid sequence of XM_{—}473199 from Oryza sativa, e.g. as shown in the respective line in column 5 of Table I, application no. 4, has been published in G. And the activity of the gene product thereof is the activity of XM_{—}473199-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “XM_{—}473199-protein”, especially from Oryza sativa or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said XM_{—}473199, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said XM_{—}473199, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said XM_{—}473199, or a functional equivalent or a homolog thereof as depicted in column 8 of Table II, application no. 4, preferably a homolog or functional equivalent as depicted in column 8 of Table II B, application no. 4, and being depicted in the same respective line as said XM_{—}473199, and preferably the activity is increased non-targeted.

Accordingly, in one embodiment, the molecule which activity is to be increased in the process of the invention is the gene product with an activity as a “XM_{—}473199-protein”, preferably it is the molecule of section (a) or (b) of this paragraph.

In particular, it was observed that in plants, especially in Arabidopsis thaliana, increasing or generating the activity of a gene product non-targeted with the activity of a “XM_{—}473199protein”, preferably being encoded by a gene comprising the nucleic acid sequence SEQ ID NO. 62244, preferably the coding region thereof, conferred the production of or the increase in threonine compared with the wild type control.

The nucleic acid sequence of Yal038w from Saccharomyces cerevisiae, e.g. as shown in the respective line in column 5 of Table I, application no. 4, has been published in A. And the activity of the gene product thereof is the activity of pyruvate kinase.

Accordingly, in one embodiment, the process of the present invention for producing threonine in a non-human organism, like a microorganism or a plant or a part thereof, comprises increasing or generating the activity of a gene product with the activity of a gene product conferring the activity of “pyruvate kinase”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Yal038w, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Yal038w, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Yal038w, or a functional equivalent or a homolog thereof as depicted in column 8 of Table II, application no. 4, preferably a homolog or functional equivalent as depicted in column 8 of Table II B, application no. 4, and being depicted in the same respective line as said Yal038w, and preferably the activity is increased plastidic.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 96022, preferably the coding region thereof, conferred the production of or the increase in threonine compared with the wild type control.

The nucleic acid sequence of Ybl021c from Saccharomyces cerevisiae, e.g. as shown in the respective line in column 5 of Table I, application no. 4, has been published in A. And the activity of the gene product thereof is the activity of transcription factor.

Accordingly, in one embodiment, the process of the present invention for producing threonine in a non-human organism, like a microorganism or a plant or a part thereof, comprises increasing or generating the activity of a gene product with the activity of a gene product conferring the activity of “transcription factor”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Ybl021c, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Ybl021 c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Ybl021c, or a functional equivalent or a homolog thereof as depicted in column 8 of Table II, application no. 4, preferably a homolog or functional equivalent as depicted in column 8 of Table II B, application no. 4, and being depicted in the same respective line as said Ybl021c, and preferably the activity is increased non-targeted.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 62524, preferably the coding region thereof, conferred the production of or the increase in threonine compared with the wild type control.

The nucleic acid sequence of Ybr160w from Saccharomyces cerevisiae, e.g. as shown in the respective line in column 5 of Table I, application no. 4, has been published in A. And the activity of the gene product thereof is the activity of cell division control protein.

Accordingly, in one embodiment, the process of the present invention for producing threonine in a non-human organism, like a microorganism or a plant or a part thereof, comprises increasing or generating the activity of a gene product with the activity of a gene product conferring the activity of “cell division control protein”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Ybr160w, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Ybr160w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Ybr160w, or a functional equivalent or a homolog thereof as depicted in column 8 of Table II, application no. 4, preferably a homolog or functional equivalent as depicted in column 8 of Table II B, application no. 4, and being depicted in the same respective line as said Ybr160w, and preferably the activity is increased non-targeted.

Accordingly, in one embodiment, the molecule which activity is to be increased in the process of the invention is the gene product with an activity as a “cell division control protein”, preferably it is the molecule of section (a) or (b) of this paragraph.

In particular, it was observed that in plants, especially in Arabidopsis thaliana, increasing or generating the activity of a gene product non-targeted with the activity of a “cell division control protein”, preferably being encoded by a gene comprising the nucleic acid sequence SEQ ID NO. 62717, preferably the coding region thereof, conferred the production of or the increase in threonine compared with the wild type control.

The nucleic acid sequence of Ybr218c from Saccharomyces cerevisiae, e.g. as shown in the respective line in column 5 of Table I, application no. 4, has been published in A. And the activity of the gene product thereof is the activity of pyruvate carboxylase.

Accordingly, in one embodiment, the process of the present invention for producing threonine in a non-human organism, like a microorganism or a plant or a part thereof, comprises increasing or generating the activity of a gene product with the activity of a gene product conferring the activity of “pyruvate carboxylase”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Ybr218c, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Ybr218c, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Ybr218c, or a functional equivalent or a homolog thereof as depicted in column 8 of Table II, application no. 4, preferably a homolog or functional equivalent as depicted in column 8 of Table II B, application no. 4, and being depicted in the same respective line as said Ybr218c, and preferably the activity is increased plastidic.

Accordingly, in one embodiment, the molecule which activity is to be increased in the process of the invention is the gene product with an activity as a “pyruvate carboxylase”, preferably it is the molecule of section (a) or (b) of this paragraph.

In particular, it was observed that in plants, especially in Arabidopsis thaliana, increasing or generating the activity of a gene product plastidic with the activity of a “pyruvate carboxylase”, preferably being encoded by a gene comprising the nucleic acid sequence SEQ ID NO. 96779, preferably the coding region thereof, conferred the production of or the increase in threonine compared with the wild type control.

The nucleic acid sequence of Ycl027w from Saccharomyces cerevisiae, e.g. as shown in the respective line in column 5 of Table I, application no. 4, has been published in A. And the activity of the gene product thereof is the activity of ycl027w-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “ycl027w-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Ycl027w, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Ycl027w, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Ycl027w, or a functional equivalent or a homolog thereof as depicted in column 8 of Table II, application no. 4, preferably a homolog or functional equivalent as depicted in column 8 of Table II B, application no. 4, and being depicted in the same respective line as said Ycl027w, and preferably the activity is increased plastidic.

Accordingly, in one embodiment, the molecule which activity is to be increased in the process of the invention is the gene product with an activity as a “ycl027w-protein”, preferably it is the molecule of section (a) or (b) of this paragraph.

In particular, it was observed that in plants, especially in Arabidopsis thaliana, increasing or generating the activity of a gene product plastidic with the activity of a “ycl027w-protein”, preferably being encoded by a gene comprising the nucleic acid sequence SEQ ID NO. 96980, preferably the coding region thereof, conferred the production of or the increase in threonine compared with the wild type control.

The nucleic acid sequence of Ydr044w from Saccharomyces cerevisiae, e.g. as shown in the respective line in column 5 of Table I, application no. 4, has been published in A. And the activity of the gene product thereof is the activity of coproporphyrinogen III oxidase.

Accordingly, in one embodiment, the process of the present invention for producing threonine in a non-human organism, like a microorganism or a plant or a part thereof, comprises increasing or generating the activity of a gene product with the activity of a gene product conferring the activity of “coproporphyrinogen III oxidase”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Ydr044w, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Ydr044w, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Ydr044w, or a functional equivalent or a homolog thereof as depicted in column 8 of Table II, application no. 4, preferably a homolog or functional equivalent as depicted in column 8 of Table II B, application no. 4, and being depicted in the same respective line as said Ydr044w, and preferably the activity is increased plastidic.

Accordingly, in one embodiment, the molecule which activity is to be increased in the process of the invention is the gene product with an activity as a “coproporphyrinogen III oxidase”, preferably it is the molecule of section (a) or (b) of this paragraph.

In particular, it was observed that in plants, especially in Arabidopsis thaliana, increasing or generating the activity of a gene product plastidic with the activity of a “coproporphyrinogen III oxidase”, preferably being encoded by a gene comprising the nucleic acid sequence SEQ ID NO. 63334, preferably the coding region thereof, conferred the production of or the increase in threonine compared with the wild type control. The nucleic acid sequence of Ydr183w from Saccharomyces cerevisiae, e.g. as shown in the respective line in column 5 of Table I, application no. 4, has been published in A. And the activity of the gene product thereof is the activity of ydr183w-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “ydr183w-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Ydr183w, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Ydr183w, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Ydr183w, or a functional equivalent or a homolog thereof as depicted in column 8 of Table II, application no. 4, preferably a homolog or functional equivalent as depicted in column 8 of Table II B, application no. 4, and being depicted in the same respective line as said Ydr183w, and preferably the activity is increased plastidic.

Accordingly, in one embodiment, the molecule which activity is to be increased in the process of the invention is the gene product with an activity as a “ydr183w-protein”, preferably it is the molecule of section (a) or (b) of this paragraph.

In particular, it was observed that in plants, especially in Arabidopsis thaliana, increasing or generating the activity of a gene product plastidic with the activity of a “ydr183w-protein”, preferably being encoded by a gene comprising the nucleic acid sequence SEQ ID NO. 90103, preferably the coding region thereof, conferred the production of or the increase in threonine compared with the wild type control.

The nucleic acid sequence of Ydr342c from Saccharomyces cerevisiae, e.g. as shown in the respective line in column 5 of Table I, application no. 4, has been published in A. And the activity of the gene product thereof is the activity of hexose transporter.

Accordingly, in one embodiment, the process of the present invention for producing threonine in a non-human organism, like a microorganism or a plant or a part thereof, comprises increasing or generating the activity of a gene product with the activity of a gene product conferring the activity of “hexose transporter”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Ydr342c, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Ydr342c, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Ydr342c, or a functional equivalent or a homolog thereof as depicted in column 8 of Table II, application no. 4, preferably a homolog or functional equivalent as depicted in column 8 of Table II B, application no. 4, and being depicted in the same respective line as said Ydr342c, and preferably the activity is increased plastidic.

Accordingly, in one embodiment, the molecule which activity is to be increased in the process of the invention is the gene product with an activity as a “hexose transporter”, preferably it is the molecule of section (a) or (b) of this paragraph.

In particular, it was observed that in plants, especially in Arabidopsis thaliana, increasing or generating the activity of a gene product plastidic with the activity of a “hexose transporter”, preferably being encoded by a gene comprising the nucleic acid sequence SEQ ID NO. 96986, preferably the coding region thereof, conferred the production of or the increase in threonine compared with the wild type control.

The nucleic acid sequence of Yel030c-a from Saccharomyces cerevisiae, e.g. as shown in the respective line in column 5 of Table I, application no. 4, has been published in A. And the activity of the gene product thereof is the activity of YEL030C-A-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “YEL030C-A-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Yel030c-a, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Yel030c-a, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Yel030c-a, or a functional equivalent or a homolog thereof as depicted in column 8 of Table II, application no. 4, preferably a homolog or functional equivalent as depicted in column 8 of Table II B, application no. 4, and being depicted in the same respective line as said Yel030c-a, and preferably the activity is increased non-targeted.

Accordingly, in one embodiment, the molecule which activity is to be increased in the process of the invention is the gene product with an activity as a “YEL030C-A-protein”, preferably it is the molecule of section (a) or (b) of this paragraph.

In particular, it was observed that in plants, especially in Arabidopsis thaliana, increasing or generating the activity of a gene product non-targeted with the activity of a “YEL030C-A-protein”, preferably being encoded by a gene comprising the nucleic acid sequence SEQ ID NO. 97294, preferably the coding region thereof, conferred the production of or the increase in threonine compared with the wild type control.

The nucleic acid sequence of Yel069c from Saccharomyces cerevisiae, e.g. as shown in the respective line in column 5 of Table I, application no. 4, has been published in A. And the activity of the gene product thereof is the activity of hexose transporter.

Accordingly, in one embodiment, the process of the present invention for producing threonine in a non-human organism, like a microorganism or a plant or a part thereof, comprises increasing or generating the activity of a gene product with the activity of a gene product conferring the activity of “hexose transporter”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Yel069c, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Yel069c, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Yel069c, or a functional equivalent or a homolog thereof as depicted in column 8 of Table II, application no. 4, preferably a homolog or functional equivalent as depicted in column 8 of Table II B, application no. 4, and being depicted in the same respective line as said Yel069c, and preferably the activity is increased plastidic.

Accordingly, in one embodiment, the molecule which activity is to be increased in the process of the invention is the gene product with an activity as a “hexose transporter”, preferably it is the molecule of section (a) or (b) of this paragraph.

In particular, it was observed that in plants, especially in Arabidopsis thaliana, increasing or generating the activity of a gene product plastidic with the activity of a “hexose transporter”, preferably being encoded by a gene comprising the nucleic acid sequence SEQ ID NO. 97298, preferably the coding region thereof, conferred the production of or the increase in threonine compared with the wild type control.

The nucleic acid sequence of Yfl054c from Saccharomyces cerevisiae, e.g. as shown in the respective line in column 5 of Table I, application no. 4, has been published in A. And the activity of the gene product thereof is the activity of yfl054c-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “yfl054c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Yfl054c, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Yfl054c, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Yfl054c, or a functional equivalent or a homolog thereof as depicted in column 8 of Table II, application no. 4, preferably a homolog or functional equivalent as depicted in column 8 of Table II B, application no. 4, and being depicted in the same respective line as said Yfl054c, and preferably the activity is increased plastidic.

Accordingly, in one embodiment, the molecule which activity is to be increased in the process of the invention is the gene product with an activity as a “yfl054c-protein”, preferably it is the molecule of section (a) or (b) of this paragraph.

In particular, it was observed that in plants, especially in Arabidopsis thaliana, increasing or generating the activity of a gene product plastidic with the activity of a “yfl054c-protein”, preferably being encoded by a gene comprising the nucleic acid sequence SEQ ID NO. 64148, preferably the coding region thereof, conferred the production of or the increase in threonine compared with the wild type control.

The nucleic acid sequence of Ygl237c from Saccharomyces cerevisiae, e.g. as shown in the respective line in column 5 of Table I, application no. 4, has been published in A. And the activity of the gene product thereof is the activity of ygl237c-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “ygl237c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Ygl237c, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Ygl237c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Ygl237c, or a functional equivalent or a homolog thereof as depicted in column 8 of Table II, application no. 4, preferably a homolog or functional equivalent as depicted in column 8 of Table II B, application no. 4, and being depicted in the same respective line as said Ygl237c, and preferably the activity is increased non-targeted.

Accordingly, in one embodiment, the molecule which activity is to be increased in the process of the invention is the gene product with an activity as a “ygl237c-protein”, preferably it is the molecule of section (a) or (b) of this paragraph.

In particular, it was observed that in plants, especially in Arabidopsis thaliana, increasing or generating the activity of a gene product non-targeted with the activity of a “ygl237c-protein”, preferably being encoded by a gene comprising the nucleic acid sequence SEQ ID NO. 64177, preferably the coding region thereof, conferred the production of or the increase in threonine compared with the wild type control.

The nucleic acid sequence of Yhr131c from Saccharomyces cerevisiae, e.g. as shown in the respective line in column 5 of Table I, application no. 4, has been published in A. And the activity of the gene product thereof is the activity of yhr131c-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “yhr131c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Yhr131c, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Yhr131c, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Yhr131c, or a functional equivalent or a homolog thereof as depicted in column 8 of Table II, application no. 4, preferably a homolog or functional equivalent as depicted in column 8 of Table II B, application no. 4, and being depicted in the same respective line as said Yhr131c, and preferably the activity is increased plastidic.

Accordingly, in one embodiment, the molecule which activity is to be increased in the process of the invention is the gene product with an activity as a “yhr131c-protein”, preferably it is the molecule of section (a) or (b) of this paragraph.

In particular, it was observed that in plants, especially in Arabidopsis thaliana, increasing or generating the activity of a gene product plastidic with the activity of a “yhr131c-protein”, preferably being encoded by a gene comprising the nucleic acid sequence SEQ ID NO. 97620, preferably the coding region thereof, conferred the production of or the increase in threonine compared with the wild type control.

The nucleic acid sequence of Yil053w from Saccharomyces cerevisiae, e.g. as shown in the respective line in column 5 of Table I, application no. 4, has been published in A. And the activity of the gene product thereof is the activity of (DL)-glycerol-3-phosphatase.

Accordingly, in one embodiment, the process of the present invention for producing threonine in a non-human organism, like a microorganism or a plant or a part thereof, comprises increasing or generating the activity of a gene product with the activity of a gene product conferring the activity of “(DL)-glycerol-3-phosphatase”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Yil053w, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Yil053w, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Yil053w, or a functional equivalent or a homolog thereof as depicted in column 8 of Table II, application no. 4, preferably a homolog or functional equivalent as depicted in column 8 of Table II B, application no. 4, and being depicted in the same respective line as said Yil053w, and preferably the activity is increased plastidic.

Accordingly, in one embodiment, the molecule which activity is to be increased in the process of the invention is the gene product with an activity as a “(DL)-glycerol-3-phosphatase”, preferably it is the molecule of section (a) or (b) of this paragraph.

In particular, it was observed that in plants, especially in Arabidopsis thaliana, increasing or generating the activity of a gene product plastidic with the activity of a “(DL)-glycerol-3-phosphatase”, preferably being encoded by a gene comprising the nucleic acid sequence SEQ ID NO. 14769, preferably the coding region thereof, conferred the production of or the increase in threonine compared with the wild type control.

The nucleic acid sequence of Yil074c from Saccharomyces cerevisiae, e.g. as shown in the respective line in column 5 of Table I, application no. 4, has been published in A. And the activity of the gene product thereof is the activity of 3-phosphoglycerate dehydrogenase.

Accordingly, in one embodiment, the process of the present invention for producing threonine in a non-human organism, like a microorganism or a plant or a part thereof, comprises increasing or generating the activity of a gene product with the activity of a gene product conferring the activity of “3-phosphoglycerate dehydrogenase”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Yil074c, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Yil074c, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Yil074c, or a functional equivalent or a homolog thereof as depicted in column 8 of Table II, application no. 4, preferably a homolog or functional equivalent as depicted in column 8 of Table II B, application no. 4, and being depicted in the same respective line as said Yil074c, and preferably the activity is increased plastidic.

Accordingly, in one embodiment, the molecule which activity is to be increased in the process of the invention is the gene product with an activity as a “3-phosphoglycerate dehydrogenase”, preferably it is the molecule of section (a) or (b) of this paragraph.

In particular, it was observed that in plants, especially in Arabidopsis thaliana, increasing or generating the activity of a gene product plastidic with the activity of a “3-phosphoglycerate dehydrogenase”, preferably being encoded by a gene comprising the nucleic acid sequence SEQ ID NO. 64563, preferably the coding region thereof, conferred the production of or the increase in threonine compared with the wild type control.

The nucleic acid sequence of Yir038c from Saccharomyces cerevisiae, e.g. as shown in the respective line in column 5 of Table I, application no. 4, has been published in A. And the activity of the gene product thereof is the activity of glutathione S-transferase.

Accordingly, in one embodiment, the process of the present invention for producing threonine in a non-human organism, like a microorganism or a plant or a part thereof, comprises increasing or generating the activity of a gene product with the activity of a gene product conferring the activity of “glutathione S-transferase”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Yir038c, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Yir038c, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Yir038c, or a functional equivalent or a homolog thereof as depicted in column 8 of Table II, application no. 4, preferably a homolog or functional equivalent as depicted in column 8 of Table II B, application no. 4, and being depicted in the same respective line as said Yir038c, and preferably the activity is increased plastidic.

Accordingly, in one embodiment, the molecule which activity is to be increased in the process of the invention is the gene product with an activity as a “glutathione S-transferase”, preferably it is the molecule of section (a) or (b) of this paragraph.

In particular, it was observed that in plants, especially in Arabidopsis thaliana, increasing or generating the activity of a gene product plastidic with the activity of a “glutathione 5transferase”, preferably being encoded by a gene comprising the nucleic acid sequence SEQ ID NO. 90306, preferably the coding region thereof, conferred the production of or the increase in threonine compared with the wild type control.

The nucleic acid sequence of Yjl073w from Saccharomyces cerevisiae, e.g. as shown in the respective line in column 5 of Table I, application no. 4, has been published in A. And the activity of the gene product thereof is the activity of DnaJ-like chaperone.

Accordingly, in one embodiment, the process of the present invention for producing threonine in a non-human organism, like a microorganism or a plant or a part thereof, comprises increasing or generating the activity of a gene product with the activity of a gene product conferring the activity of “DnaJ-like chaperone”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Yjl073w, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Yjl073w, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Yjl073w, or a functional equivalent or a homolog thereof as depicted in column 8 of Table II, application no. 4, preferably a homolog or functional equivalent as depicted in column 8 of Table II B, application no. 4, and being depicted in the same respective line as said Yjl073w, and preferably the activity is increased plastidic.

Accordingly, in one embodiment, the molecule which activity is to be increased in the process of the invention is the gene product with an activity as a “DnaJ-like chaperone”, preferably it is the molecule of section (a) or (b) of this paragraph.

In particular, it was observed that in plants, especially in Arabidopsis thaliana, increasing or generating the activity of a gene product plastidic with the activity of a “DnaJ-like chaperone”, preferably being encoded by a gene comprising the nucleic acid sequence SEQ ID NO. 64964, preferably the coding region thereof, conferred the production of or the increase in threonine compared with the wild type control.

The nucleic acid sequence of Yjl127w-a from Saccharomyces cerevisiae, e.g. as shown in the respective line in column 5 of Table I, application no. 4, has been published in A. And the activity of the gene product thereof is the activity of YJL127W-A-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “YJL127W-A-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Yjl127w-a, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Yjl127w-a, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Yjl127w-a, or a functional equivalent or a homolog thereof as depicted in column 8 of Table II, application no. 4, preferably a homolog or functional equivalent as depicted in column 8 of Table II B, application no. 4, and being depicted in the same respective line as said Yjl127w-a, and preferably the activity is increased non-targeted.

Accordingly, in one embodiment, the molecule which activity is to be increased in the process of the invention is the gene product with an activity as a “YJL127W-A-protein”, preferably it is the molecule of section (a) or (b) of this paragraph.

In particular, it was observed that in plants, especially in Arabidopsis thaliana, increasing or generating the activity of a gene product non-targeted with the activity of a “YJL127W-A-protein”, preferably being encoded by a gene comprising the nucleic acid sequence SEQ ID NO. 97631, preferably the coding region thereof, conferred the production of or the increase in threonine compared with the wild type control.

The nucleic acid sequence of Yjl219w from Saccharomyces cerevisiae, e.g. as shown in the respective line in column 5 of Table I, application no. 4, has been published in A. And the activity of the gene product thereof is the activity of hexose transporter.

Accordingly, in one embodiment, the process of the present invention for producing threonine in a non-human organism, like a microorganism or a plant or a part thereof, comprises increasing or generating the activity of a gene product with the activity of a gene product conferring the activity of “hexose transporter”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Yjl219w, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Yjl219w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Yjl219w, or a functional equivalent or a homolog thereof as depicted in column 8 of Table II, application no. 4, preferably a homolog or functional equivalent as depicted in column 8 of Table II B, application no. 4, and being depicted in the same respective line as said Yjl219w, and preferably the activity is increased non-targeted.

Accordingly, in one embodiment, the molecule which activity is to be increased in the process of the invention is the gene product with an activity as a “hexose transporter”, preferably it is the molecule of section (a) or (b) of this paragraph.

In particular, it was observed that in plants, especially in Arabidopsis thaliana, increasing or generating the activity of a gene product non-targeted with the activity of a “hexose transporter”, preferably being encoded by a gene comprising the nucleic acid sequence SEQ ID NO. 97635, preferably the coding region thereof, conferred the production of or the increase in threonine compared with the wild type control.

The nucleic acid sequence of Yjr139c from Saccharomyces cerevisiae, e.g. as shown in the respective line in column 5 of Table I, application no. 4, has been published in A. And the activity of the gene product thereof is the activity of homoserine dehydrogenase.

Accordingly, in one embodiment, the process of the present invention for producing threonine in a non-human organism, like a microorganism or a plant or a part thereof, comprises increasing or generating the activity of a gene product with the activity of a gene product conferring the activity of “homoserine dehydrogenase”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Yjr139c, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Yjr139c, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Yjr139c, or a functional equivalent or a homolog thereof as depicted in column 8 of Table II, application no. 4, preferably a homolog or functional equivalent as depicted in column 8 of Table II B, application no. 4, and being depicted in the same respective line as said Yjr139c, and preferably the activity is increased plastidic.

Accordingly, in one embodiment, the molecule which activity is to be increased in the process of the invention is the gene product with an activity as a “homoserine dehydrogenase”, preferably it is the molecule of section (a) or (b) of this paragraph.

In particular, it was observed that in plants, especially in Arabidopsis thaliana, increasing or generating the activity of a gene product plastidic with the activity of a “homoserine dehydrogenase”, preferably being encoded by a gene comprising the nucleic acid sequence SEQ ID NO. 14885, preferably the coding region thereof, conferred the production of or the increase in threonine compared with the wild type control.

The nucleic acid sequence of Yjr148w from Saccharomyces cerevisiae, e.g. as shown in the respective line in column 5 of Table I, application no. 4, has been published in A. And the activity of the gene product thereof is the activity of branched chain aminotransferase.

Accordingly, in one embodiment, the process of the present invention for producing threonine in a non-human organism, like a microorganism or a plant or a part thereof, comprises increasing or generating the activity of a gene product with the activity of a gene product conferring the activity of “branched chain aminotransferase”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Yjr148w, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Yjr148w, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Yjr148w, or a functional equivalent or a homolog thereof as depicted in column 8 of Table II, application no. 4, preferably a homolog or functional equivalent as depicted in column 8 of Table II B, application no. 4, and being depicted in the same respective line as said Yjr148w, and preferably the activity is increased plastidic.

Accordingly, in one embodiment, the molecule which activity is to be increased in the process of the invention is the gene product with an activity as a “branched chain aminotransferase”, preferably it is the molecule of section (a) or (b) of this paragraph.

In particular, it was observed that in plants, especially in Arabidopsis thaliana, increasing or generating the activity of a gene product plastidic with the activity of a “branched chain aminotransferase”, preferably being encoded by a gene comprising the nucleic acid sequence SEQ ID NO. 90409, preferably the coding region thereof, conferred the production of or the increase in threonine compared with the wild type control.

The nucleic acid sequence of YKL038W from Saccharomyces cerevisiae, e.g. as shown in the respective line in column 5 of Table I, application no. 4, has been published in A. And the activity of the gene product thereof is the activity of YKL038W-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “YKL038W-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said YKL038W, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said YKL038W, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said YKL038W, or a functional equivalent or a homolog thereof as depicted in column 8 of Table II, application no. 4, preferably a homolog or functional equivalent as depicted in column 8 of Table II B, application no. 4, and being depicted in the same respective line as said YKL038W, and preferably the activity is increased plastidic.

Accordingly, in one embodiment, the molecule which activity is to be increased in the process of the invention is the gene product with an activity as a “YKL038W-protein”, preferably it is the molecule of section (a) or (b) of this paragraph.

In particular, it was observed that in plants, especially in Arabidopsis thaliana, increasing or generating the activity of a gene product plastidic with the activity of a “YKL038W-protein”, preferably being encoded by a gene comprising the nucleic acid sequence SEQ ID NO. 90602, preferably the coding region thereof, conferred the production of or the increase in threonine compared with the wild type control.

The nucleic acid sequence of Ykr097w from Saccharomyces cerevisiae, e.g. as shown in the respective line in column 5 of Table I, application no. 4, has been published in A. And the activity of the gene product thereof is the activity of phosphoenolpyruvate carboxykinase.

Accordingly, in one embodiment, the process of the present invention for producing threonine in a non-human organism, like a microorganism or a plant or a part thereof, comprises increasing or generating the activity of a gene product with the activity of a gene product conferring the activity of “phosphoenolpyruvate carboxykinase”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Ykr097w, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Ykr097w, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Ykr097w, or a functional equivalent or a homolog thereof as depicted in column 8 of Table II, application no. 4, preferably a homolog or functional equivalent as depicted in column 8 of Table II B, application no. 4, and being depicted in the same respective line as said Ykr097w, and preferably the activity is increased plastidic.

Accordingly, in one embodiment, the molecule which activity is to be increased in the process of the invention is the gene product with an activity as a “phosphoenolpyruvate carboxykinase”, preferably it is the molecule of section (a) or (b) of this paragraph.

In particular, it was observed that in plants, especially in Arabidopsis thaliana, increasing or generating the activity of a gene product plastidic with the activity of a “phosphoenolpyruvate carboxykinase”, preferably being encoded by a gene comprising the nucleic acid sequence SEQ ID NO. 98075, preferably the coding region thereof, conferred the production of or the increase in threonine compared with the wild type control.

The nucleic acid sequence of Ylr027c from Saccharomyces cerevisiae, e.g. as shown in the respective line in column 5 of Table I, application no. 4, has been published in A. And the activity of the gene product thereof is the activity of aspartate aminotransferase.

Accordingly, in one embodiment, the process of the present invention for producing threonine in a non-human organism, like a microorganism or a plant or a part thereof, comprises increasing or generating the activity of a gene product with the activity of a gene product conferring the activity of “aspartate aminotransferase”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Ylr027c, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Ylr027c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Ylr027c, or a functional equivalent or a homolog thereof as depicted in column 8 of Table II, application no. 4, preferably a homolog or functional equivalent as depicted in column 8 of Table II B, application no. 4, and being depicted in the same respective line as said Ylr027c, and preferably the activity is increased non-targeted.

Accordingly, in one embodiment, the molecule which activity is to be increased in the process of the invention is the gene product with an activity as a “aspartate aminotransferase”, preferably it is the molecule of section (a) or (b) of this paragraph.

In particular, it was observed that in plants, especially in Arabidopsis thaliana, increasing or generating the activity of a gene product non-targeted with the activity of a “aspartate aminotransferase”, preferably being encoded by a gene comprising the nucleic acid sequence SEQ ID NO. 90615, preferably the coding region thereof, conferred the production of or the increase in threonine compared with the wild type control.

The nucleic acid sequence of Ylr348c from Saccharomyces cerevisiae, e.g. as shown in the respective line in column 5 of Table I, application no. 4, has been published in A. And the activity of the gene product thereof is the activity of mitochondrial dicarboxylate transporter.

Accordingly, in one embodiment, the process of the present invention for producing threonine in a non-human organism, like a microorganism or a plant or a part thereof, comprises increasing or generating the activity of a gene product with the activity of a gene product conferring the activity of “mitochondrial dicarboxylate transporter”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Ylr348c, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Ylr348c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Ylr348c, or a functional equivalent or a homolog thereof as depicted in column 8 of Table II, application no. 4, preferably a homolog or functional equivalent as depicted in column 8 of Table II B, application no. 4, and being depicted in the same respective line as said Ylr348c, and preferably the activity is increased non-targeted.

Accordingly, in one embodiment, the molecule which activity is to be increased in the process of the invention is the gene product with an activity as a “mitochondrial dicarboxylate transporter”, preferably it is the molecule of section (a) or (b) of this paragraph.

In particular, it was observed that in plants, especially in Arabidopsis thaliana, increasing or generating the activity of a gene product non-targeted with the activity of a “mitochondrial dicarboxylate transporter”, preferably being encoded by a gene comprising the nucleic acid sequence SEQ ID NO. 98243, preferably the coding region thereof, conferred the production of or the increase in threonine compared with the wild type control.

The nucleic acid sequence of Yml123c from Saccharomyces cerevisiae, e.g. as shown in the respective line in column 5 of Table I, application no. 4, has been published in A. And the activity of the gene product thereof is the activity of phosphate transporter.

Accordingly, in one embodiment, the process of the present invention for producing threonine in a non-human organism, like a microorganism or a plant or a part thereof, comprises increasing or generating the activity of a gene product with the activity of a gene product conferring the activity of “phosphate transporter”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Yml123c, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Yml123c, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Yml123c, or a functional equivalent or a homolog thereof as depicted in column 8 of Table II, application no. 4, preferably a homolog or functional equivalent as depicted in column 8 of Table II B, application no. 4, and being depicted in the same respective line as said Yml123c, and preferably the activity is increased plastidic.

Accordingly, in one embodiment, the molecule which activity is to be increased in the process of the invention is the gene product with an activity as a “phosphate transporter”, preferably it is the molecule of section (a) or (b) of this paragraph.

In particular, it was observed that in plants, especially in Arabidopsis thaliana, increasing or generating the activity of a gene product plastidic with the activity of a “phosphate transporter”, preferably being encoded by a gene comprising the nucleic acid sequence SEQ ID NO. 91785, preferably the coding region thereof, conferred the production of or the increase in threonine compared with the wild type control.

The nucleic acid sequence of Yol045w from Saccharomyces cerevisiae, e.g. as shown in the respective line in column 5 of Table I, application no. 4, has been published in A. And the activity of the gene product thereof is the activity of protein kinase.

Accordingly, in one embodiment, the process of the present invention for producing threonine in a non-human organism, like a microorganism or a plant or a part thereof, comprises increasing or generating the activity of a gene product with the activity of a gene product conferring the activity of “protein kinase”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Yol045w, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Yol045w, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Yol045w, or a functional equivalent or a homolog thereof as depicted in column 8 of Table II, application no. 4, preferably a homolog or functional equivalent as depicted in column 8 of Table II B, application no. 4, and being depicted in the same respective line as said Yol045w, and preferably the activity is increased plastidic.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product plastidic with the activity of a “protein kinase”, preferably being encoded by a gene comprising the nucleic acid sequence SEQ ID NO. 67684, preferably the coding region thereof, conferred the production of or the increase in threonine compared with the wild type control.

The nucleic acid sequence of Yor244w from Saccharomyces cerevisiae, e.g. as shown in the respective line in column 5 of Table I, application no. 4, has been published in A. And the activity of the gene product thereof is the activity of histone acetyltransferase.

Accordingly, in one embodiment, the process of the present invention for producing threonine in a non-human organism, like a microorganism or a plant or a part thereof, comprises increasing or generating the activity of a gene product with the activity of a gene product conferring the activity of “histone acetyltransferase”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Yor244w, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Yor244w, and preferably the activity is increased plastidic, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Yor244w, or a functional equivalent or a homolog thereof as depicted in column 8 of Table II, application no. 4, preferably a homolog or functional equivalent as depicted in column 8 of Table II B, application no. 4, and being depicted in the same respective line as said Yor244w, and preferably the activity is increased plastidic.

Accordingly, in one embodiment, the molecule which activity is to be increased in the process of the invention is the gene product with an activity as a “histone acetyltransferase”, preferably it is the molecule of section (a) or (b) of this paragraph.

In particular, it was observed that in plants, especially in Arabidopsis thaliana, increasing or generating the activity of a gene product plastidic with the activity of a “histone acetyltransferase”, preferably being encoded by a gene comprising the nucleic acid sequence SEQ ID NO. 92540, preferably the coding region thereof, conferred the production of or the increase in threonine compared with the wild type control.

The nucleic acid sequence of Ypr119w from Saccharomyces cerevisiae, e.g. as shown in the respective line in column 5 of Table I, application no. 4, has been published in A. And the activity of the gene product thereof is the activity of cyclin.

Accordingly, in one embodiment, the process of the present invention for producing threonine in a non-human organism, like a microorganism or a plant or a part thereof, comprises increasing or generating the activity of a gene product with the activity of a gene product conferring the activity of “cyclin”, especially from Saccharomyces cerevisiae or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Ypr119w, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Ypr119w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Ypr119w, or a functional equivalent or a homolog thereof as depicted in column 8 of Table II, application no. 4, preferably a homolog or functional equivalent as depicted in column 8 of Table II B, application no. 4, and being depicted in the same respective line as said Ypr119w, and preferably the activity is increased non-targeted.

Accordingly, in one embodiment, the molecule which activity is to be increased in the process of the invention is the gene product with an activity as a “cyclin”, preferably it is the molecule of section (a) or (b) of this paragraph.

In particular, it was observed that in plants, especially in Arabidopsis thaliana, increasing or generating the activity of a gene product non-targeted with the activity of a “cyclin”, preferably being encoded by a gene comprising the nucleic acid sequence SEQ ID NO. 98445, preferably the coding region thereof, conferred the production of or the increase in threonine compared with the wild type control.

The nucleic acid sequence of Zm_{—}4842_BE510522 from Zea mays, e.g. as shown in the respective line in column 5 of Table I, application no. 4, is unpublished. And the activity of the gene product thereof is the activity of Zm_{—}4842_BE510522-protein.

Accordingly, in one embodiment, the process of the present invention for producing threonine in a non-human organism, like a microorganism or a plant or a part thereof, comprises increasing or generating the activity of a gene product with the activity of a gene product conferring the activity of “Zm_{—}4842_BE510522-protein”, especially from Zea mays or its functional equivalent or its homolog, e.g. the increase of
(a) a gene product of a gene comprising the nucleic acid molecule as shown in the respective line in column 5 of Table I, application no. 4, preferably the coding region thereof, or a homolog or a fragment thereof, and being depicted in the same respective line as said Zm_{—}4842_BE510522, or a functional equivalent or a homolog thereof as shown in column 8 of Table I, application no. 4, preferably the coding region thereof, particularly a homolog or functional equivalent as shown in column 8 of Table I B, application no. 4, and being depicted in the same respective line as said Zm_{—}4842_BE510522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 4, respectively, and being depicted in the same respective line as said Zm_{—}4842_BE510522, or a functional equivalent or a homolog thereof as depicted in column 8 of Table II, application no. 4, preferably a homolog or functional equivalent as depicted in column 8 of Table II B, application no. 4, and being depicted in the same respective line as said Zm_{—}4842_BE510522, and preferably the activity is increased non-targeted.

Accordingly, in one embodiment, the molecule which activity is to be increased in the process of the invention is the gene product with an activity as a “Zm_{—}4842_BE510522-protein”, preferably it is the molecule of section (a) or (b) of this paragraph.

In particular, it was observed that in plants, especially in Arabidopsis thaliana, increasing or generating the activity of a gene product non-targeted with the activity of a “Zm_{—}4842_BE510522-protein”, preferably being encoded by a gene comprising the nucleic acid sequence SEQ ID NO. 68413, preferably the coding region thereof, conferred the production of or the increase in threonine compared with the wild type control.

[0105.1.1.4] to [0107.1.1.4] for the disclosure of these paragraphs see [0105.1.1.1] to [0107.1.1.1] above.

A protein having an activity conferring an increase in the amount or level of the fine chemical threonine, upon targeting to the plastids or mitochondria or upon non-targeting, preferably has the structure of the respective polypeptide described herein, in particular of the polypeptides comprising the consensus sequence or at least one polypeptide motifs as shown in the respective line in Table IV, application no. 4, column 8 or of the polypeptide comprising an amino acid sequence as disclosed in the respective line in Table II, application no. 4, columns 5 or 8, or homologs or fragments thereof as described herein, or is encoded by the nucleic acid molecule characterized herein or the nucleic acid molecule according to the invention, for example by the nucleic acid molecule as shown in the respective line in Table I, application no. 4, columns 5 or 8, preferably the coding region thereof, or homologs or fragments thereof and has the herein mentioned activity.

[0109.1.1.4] to [0110.1.1.4] for the disclosure of these paragraphs see [0109.1.1.1] to [0110.1.1.1] above.

In one embodiment, the process of the present invention comprises one or more of the following steps
(a) stabilizing a protein conferring the generation or increased expression of a protein encoded by the nucleic acid molecule of the invention or of the polypeptide of the invention, e.g. of a polypeptide having the activity selected from the group consisting of (DL)-glycerol-3-phosphatase, 2,3-dihydroxyphenylpropionate 1,2-dioxygenase, 2-C-methyl-D-erythritol 4-phosphate cytidylyltransferase, 2-oxoglutarate dehydrogenase E1 subunit, 3-phosphoglycerate dehydrogenase, 47266012-protein, 49747384_SOYBEAN-protein, ABC metal ion transporter substrate-binding protein, ABC transporter permease protein, acetolactate synthase small subunit, acetyltransferase, acyl-CoA dehydrogenase, acyl-CoA synthase, argininosuccinate lyase, aspartate aminotransferase, aspartate kinase, At1g09680-protein, At4g32480-protein, At5g16650-protein, ATP synthase subunit beta, ATP-binding component of a transport system, AX653549-protein,b1003-protein, b1108p-rotein, b1445-protein,b1522-protein,b1670-protein,b1930-protein, b2360-protein, b2399-protein, b2513-protein, b2613-protein, b2739-protein, b2812-protein, b3083-protein, B3442-protein, b3646-protein, b3989-protein, b4029-protein, b4056-protein, b4250-protein, beta-galactosidase, beta-hydroxylase, bifunctional aspartokinase/homoserine dehydrogenase, bifunctional purine biosynthesis protein, branched chain amino acid ABC transporter ATP-binding protein, branched chain aminotransferase, branched-chain amino acid ABC transporter permease protein, calcium-dependent protein kinase, carbamoyl-phosphate synthase subunit, CCAAT-binding transcription factor, CDP-diacylglycerol-serine O-phosphatidyltransferase, cell division control protein, cell division protein, Chaperone protein CIpB, coproporphyrinogen III oxidase, cyclin, cyclin D , D-3-phosphoglycerate dehydrogenase, dihydrolipoamide acetyltransferase, dihydroxyacid dehydratase, DNA helicase II, DnaJ-like chaperone, D-serine dehydratase, elongation factor Tu, enoyl-CoA hydratase, enterobacterial common antigen polymerase, eukaryotic translation initiation factor 5, ferredoxin, GDP-mannose dehydrogenase, geranylgeranyl pyrophosphate synthase, gibberellin 20-oxidase, glucose dehydrogenase, glucose-6-phosphate 1-dehydrogenase, glutamate-ammonia-ligase, glutaredoxin, glutathione S-transferase, glycogen (starch) synthase, glycogen synthase, glycoprotease, glycosyl transferase, heat shock protein, heat shock transcription factor, hexose transporter, histone acetyltransferase, homocitrate synthase, homoserine dehydrogenase, hydrolase, isochorismate synthase, ketol-acid reductoisomerase, kinase, L-asparaginase, major facilitator superfamily transporter protein, malate dehydrogenase, malic enzyme, membrane transport protein, metal-dependent hydrolase, methyltransferase, mitochondrial dicarboxylate transporter, monothiol glutaredoxin, monthiol glutaredoxin, murein transglycosylase, NADP-dependent malic enzyme, nitrate/nitrite transport protein, oxireductase, peptidase, permease protein of peptide ABC transporter, phosphate transporter, phosphoanhydride phosphorylase, phosphoenolpyruvate carboxykinase, phosphoribosylaminoimidazole carboxylase catalytic subunit, photosystem II protein, polyphosphate kinase, protein kinase, protein phosphatase, pyruvate carboxylase, pyruvate kinase, restriction alleviation protein, sec-independent protein translocase, sensor histidine kinase, serine protease, sll0418-protein, slr0383-protein, succinyl-CoA synthetase beta chain, thioredoxin, threonine synthase, transcription factor, transcriptional regulator, transcriptional regulator protein, transport protein, TTC0881-protein, uroporphyrin-III C-methyltransferase, valine-pyruvate transaminase, XM_{—}473199-protein, ycl027w-protein, ydr183w-protein, YEL030C-A-protein, yfl054c-protein, ygl237c-protein, yhr131c-protein, YJL127W-A-protein, YKL038W-protein, zinc finger protein, and Zm_{—}4842_BE510522-protein, or of a polypeptide as indicated in the respective line in Table II, application no. 4, columns 5 or 8, or its homologs or fragments, and conferring the production of or an increase in threonine, respectively; and/or
(b) stabilizing a mRNA conferring the generation or increased expression of a FCRP, e.g. protein encoded by the nucleic acid molecule of the invention or its homologs or fragments, or of a mRNA encoding the polypeptide of the present invention having the hereinmentioned activity selected from the group consisting of said activities mentioned in (a) and conferring the production of or an increase in threonine, respectively; and/or
(c) increasing the specific activity of a protein conferring the increased expression of a FCRP, e.g. a protein encoded by the nucleic acid molecule of the invention or of the polypeptide of the present invention having herein-mentioned threonine generating or increasing activity, respectively, e.g. of a polypeptide having the activity of a protein as indicated in the respective line in Table II, application no. 4, columns 5 or 8, or its homologs or fragments, or decreasing the inhibitory regulation of the polypeptide of the invention; and/or
(d) generating or increasing the expression of an endogenous or artificial transcription factor mediating the expression of a protein conferring the generation or increased expression of a FCRP, e.g. a protein encoded by the nucleic acid molecule of the invention or of the polypeptide of the invention having herein-mentioned activity selected from the group consisting of said activities mentioned in (a) and conferring a threonine increasing activity, respectively, e.g. of a polypeptide having the activity of a protein as indicated in the respective line in Table II, application no. 4, columns 5 or 8 or its homologs; and/or
(e) stimulating activity of a protein conferring the increased expression of a FCRP, e.g. a protein encoded by the nucleic acid molecule of the present invention or a polypeptide of the present invention having herein-mentioned activity selected from the group consisting of said activities mentioned in (a) and conferring a threonine increasing activity, respectively, e.g. of a polypeptide having the activity of a protein as indicated in the respective line in Table II, application no. 4, columns 5 or 8, or its homologs or fragments, by adding one or more exogenous inducing factors to the non-human organism or parts thereof; and/or
(f) expressing a transgenic gene encoding a protein conferring the increased expression of a FCRP, e.g. a polypeptide encoded by the nucleic acid molecule of the present invention or a polypeptide of the present invention, having herein-mentioned activity selected from the group consisting of said activities mentioned in (a) and conferring a threonine increasing activity, respectively, e.g. of a polypeptide having the activity of a protein as indicated in the respective line in Table II, application no. 4, columns 5 or 8, or its homologs or fragments; and/or
(g) increasing the copy number of a gene conferring the increased expression of a nucleic acid molecule encoding a FCRP, e.g. a polypeptide encoded by the nucleic acid molecule of the invention or the polypeptide of the invention having herein-mentioned activity selected from the group consisting of said activities mentioned in (a) and conferring a threonine increasing activity, respectively, e.g. of a polypeptide having the activity of a protein as indicated in the respective line in Table II, application no. 4, columns 5 or 8, or its homologs or fragments; and/or
(h) increasing the expression of the endogenous gene encoding the FCRP, polypeptide of the invention, e.g. a polypeptide encoded by the nucleic acid molecule of the invention or the polypeptide of the invention having herein-mentioned activity selected from the group consisting of said activities mentioned in (a) and conferring a threonine increasing activity, respectively, e.g. of a polypeptide having the activity of a protein as indicated in the respective line in Table II, application no. 4, columns 5 or 8, or its homologs or fragments, by adding positive expression or removing negative expression elements, e.g. homologous recombination can be used to either introduce positive regulatory elements like for plants the 35S enhancer into the promoter or to remove repressor elements form regulatory regions. Further gene conversion methods can be used to disrupt repressor elements or to enhance to activity of positive elements. Positive elements can be randomly introduced in plants by T-DNA or transposon mutagenesis and lines can be identified in which the positive elements have be integrated near to a gene of the invention, the expression of which is thereby enhanced; and/or
(i) modulating growth conditions of the non-human organism in such a manner, that the expression or activity of the gene encoding the FCRP, e.g. a protein of the invention or the protein itself is enhanced, for example microorganisms or plants can be grown for example under a higher temperature regime leading to an enhanced expression of heat shock proteins, which can lead to an enhanced threonine production, respectively; and/or
(j) selecting of non-human organisms with especially high activity of the proteins of the invention from natural or from mutagenized resources and breeding them into the target organisms, e.g. the elite crops; and/or
(k) directing a protein encoded by the nucleic acid molecule of the invention or of the polypeptide of the present invention having herein-mentioned activity selected from the group consisting of said activities mentioned in (a) and conferring a threonine increasing activity, respectively, e.g. of a polypeptide having the activity of a protein as indicated in the respective line in Table II, application no. 4, columns 5 or 8, or its homologs or fragments, if for the corresponding nucleic acid molecule in the respective line in column 6 of Table I the term “plastidic” is indicated, to the plastids by the addition of a plastidial targeting sequence or if for the corresponding nucleic acid molecule in the respective line in column 6 of Table I the term “mitochondrial” is indicated, to the mitochondria by the addition of a mitochondrial targeting sequence; and/or
(l) generating the expression of a protein encoded by the nucleic acid molecule of the invention or of the polypeptide of the present invention having herein-mentioned activity selected from the group consisting of said activities mentioned in (a) and conferring a threonine increasing activity, respectively, e.g. of a polypeptide having the activity of a protein as indicated in the respective line in Table II, application no. 4, columns 5 or 8, or its homologs or fragments, if for the corresponding nucleic acid molecule in the respective line in column 6 of Table I the term “plastidic” or “mitochondrial” is indicated, in these organelles by the stable or transient transformation, advantageously stable transformation, of organelles, preferably plastids or mitochondria, with an inventive nucleic acid sequence preferably in form of an expression cassette containing said sequence leading to the expression of the nucleic acids or polypeptides of the invention in the respective organelle; and/or
(m) generating the expression of a protein encoded by the nucleic acid molecule of the invention or of the polypeptide of the present invention having herein-mentioned activity selected from the group consisting of said activities mentioned in (a) and conferring a threonine increasing activity, respectively, e.g. of a polypeptide having the activity of a protein as indicated in the respective line in Table II, application no. 4, columns 5 or 8, or its homologs or fragments, if for the corresponding nucleic acid molecule in the respective line in column 6 of Table I the term “plastidic” or “mitochondrial” is indicated, in these organelles by integration of a nucleic acid of the invention into the genome of the respective organelle under control of preferable a promoter selective for the respective organelle.

Preferably, said mRNA is the coding region of the nucleic acid molecule of the present invention and/or the protein conferring the increased expression of a protein encoded by the coding region of the nucleic acid molecule of the present invention alone or linked to a transit nucleic acid sequence or transit peptide encoding nucleic acid sequence or the polypeptide having the herein mentioned activity, e.g. conferring the generation of or increase of threonine, respectively, after increasing the expression or activity of the encoded polypeptide, non-targeted or in organelles such as plastids and/or mitochondria, preferably plastids, or having the activity of a polypeptide having an activity as the protein as shown in the respective line in Table II, application no. 4, column 3, or its homologs. Preferably the increase of threonine, respectively, takes place non-targeted or in plastids and/or mitochondria, preferably non-targeted or in plastids.

[0113.1.1.4] to [0122.1.1.4] for the disclosure of these paragraphs see [0113.1.1.1] to [0122.1.1.1] above.

The activation of an endogenous polypeptide having above-mentioned activity, e.g. having the activity of a protein as shown in the respective line in Table II, application no. 4, column 3 or of the polypeptide of the invention, e.g. conferring the increase of the fine chemical threonine, respectively, by increase of expression or activity in the cytoplasm, and/or in the cytosol, and/or in an organelle, such as plastids or mitochondria, can also be increased by introducing a synthetic transcription factor, which binds close to the coding region of the gene encoding the protein as shown in the respective line in Table II, application no. 4, column 5 or 8, or homologs or fragments thereof, and activates its transcription. A chimeric zinc finger protein can be constructed, which comprises a specific DNA-binding domain and an activation domain as e.g. the VP16 domain of Herpes Simplex virus. The specific binding domain can bind to the regulatory region of the gene encoding the protein as shown in the respective line in Table II, application no. 4, column 5 or 8, or homologs or fragments thereof. The expression of the chimeric transcription factor in a non-human organism, in particular in a plant, leads to a specific expression of the protein as shown in the respective line in Table II, application no. 4, column 5 or 8, or homologs or fragments thereof, see e.g. in WO01/52620, Oriz, Proc. Natl. Acad. Sci. USA 99, 13290 (2002), or Guan, Proc. Natl. Acad. Sci. USA 99, 13296 (2002).

[0124.1.1.4] to [0127.1.1.4] for the disclosure of these paragraphs see [0124.1.1.1] to [0127.1.1.1] above.

Owing to the introduction of a gene or a plurality of genes conferring the expression of the nucleic acid molecule of the invention or the polypeptide of the invention, for example the nucleic acid construct mentioned below, or encoding the protein as shown in the respective line in Table II, application no. 4, column 5 or 8, or homologs or fragments thereof, into a non-human organism alone or in combination with other genes, it is possible not only to increase the biosynthetic flux towards the end product, but also to increase, modify or create de novo an advantageous, preferably novel metabolites composition in the non-human organism, e.g. an advantageous composition comprising a higher content of (from a viewpoint of nutritional physiology limited) threonine and if desired other amino acids, and/or other metabolites, in free or bound form.

for the disclosure of this paragraph see [0129.1.1.1] above.

Accordingly, in one embodiment, the process according to the invention relates to a process, which comprises:
(a) providing a non-human organism, preferably a microorganism, a plant cell, a plant tissue, a plant or a part thereof, more preferably a microorganism, a plant tissue, a plant or a part thereof;
(b) increasing the activity of a protein as shown in the respective line in Table II, application no. 4, column 3 or of a polypeptide being encoded by the respective nucleic acid molecule of the present invention and described below, e.g. conferring an increase of the fine chemical threonine, respectively, in the non-human organism, preferably in the microorganism, the plant cell, the plant tissue, the plant or a part thereof, more preferably a microorganism, a plant tissue, a plant or a part thereof, especially cytoplasmic or in an organelle, like plastids or mitochondria,
(c) growing the non-human organism, preferably the microorganism, the plant cell, the plant tissue, the plant or a part thereof, under conditions which permit the production of said fine chemical in the non-human organism, preferably the microorganism, the plant cell, the plant tissue, the plant or a part thereof; and
(d) if desired, recovering, optionally isolating, said fine chemical, in free and/or bound form, and, optionally further free and/or bound amino acids, and/or other metabolites synthesized by the non-human organism, the microorganism, the plant cell, the plant tissue, the plant or a part thereof.

[0131.1.1.4] to [0139.1.1.4] for the disclosure of these paragraphs see [0131.1.1.1] to [0139.1.1.1] above.

In another embodiment, the invention relates to a (isolated) nucleic acid molecule, which comprises a nucleic acid molecule selected from the group consisting of:
(a) a nucleic acid molecule encoding a polypeptide shown in Table II, application no. 4, column 5 or 8, preferably shown in Table II B, application no. 4, column 8, or a homolog or a fragment thereof;
(b) a nucleic acid molecule shown in Table I, application no. 4, column 5 or 8, preferably shown in Table I B, application no. 4, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 4, preferably in column 8 of Table II B, application no. 4;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 4, preferably shown in column 8 of Table I B, application no. 4, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 4;
(i) a nucleic acid molecule encoding a polypeptide having the activity represented by a respective protein as depicted in column 5 of Table II, application no. 4;
(j) a nucleic acid molecule comprising a polynucleotide which is obtained by amplifying a cDNA library or a genomic library using the primers in Table III, application no. 4, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 4.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II application no. 4 and confers a generation or an increase of the respective fine chemical in a non-human organism or a part thereof as compared to a corresponding wild-type non-human organism or part thereof.

In another embodiment, the invention relates to a (isolated) nucleic acid molecule, which comprises a nucleic acid molecule selected from the group consisting of:
(a) a nucleic acid molecule encoding a polypeptide shown in Table II, column 5 or 8, application no. 4, preferably shown in Table II A, application no. 4, in column 5 or in Table II A, application no. 4, column 8 or in Table II B, application no. 4, column 8, or a homolog or a fragment thereof;
(b) a nucleic acid molecule shown in Table I, application no. 4, column 5 or 8, preferably shown in Table I A, application no. 4, in column 5 or in Table I A, application no. 4, column 8 or in Table I B, application no. 4, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 4, preferably shown in Table II A, application no. 4, in column 5 or in Table II A, application no. 4, column 8 or in Table II B, application no. 4, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 4, preferably shown in Table I A, application no. 4, in column 5 or in Table I A, application no. 4, column 8 or in Table I B column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 4;
(i) a nucleic acid molecule encoding a polypeptide having the activity represented by a respective protein as depicted in column 5 of Table II, application no. 4;
(j) a nucleic acid molecule comprising a polynucleotide which is obtained by amplifying a cDNA library or a genomic library using the primers in Table III, application no. 4, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt,500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
   or a nucleic acid molecule comprising a sequence which is complementary thereto;
   whereby, preferably, the nucleic acid molecule according to (a), (b), (c), (d), (e), (f), (g), (h), (i), (j) or (k) distinguishes over the sequence depicted in Table I A and/or I B, application no. 4, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 4, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 4, column 5 or 8, or the coding region thereof, respectively. In a further embodiment the nucleic acid molecule does not encode the polypeptide sequence shown in Table II A and/or II B, application no. 4, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 4, column 5 or 8 and does not encode a protein of the sequence shown in Table II A and/or II B, application no. 4, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 4, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 4, column 5 or 8 but less than 100%, preferably less than 99.999%, 99.99%, 99.9%, 99%, 98%, 97%, 96% or 95% identical to the sequence shown in Table II A and/or II B, application no. 4, column 5 or 8.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 4.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II, application no. 4, and confers a generation or an increase of the respective fine chemical in a non-human organism or a part thereof as compared to a corresponding wild-type non-human organism or part thereof.

[0144.1.1.4] to [0155.1.1.4] for the disclosure of these paragraphs see [0144.1.1.1] to [0155.1.1.1] above.

In a preferred embodiment a nucleic acid construct, for example an expression cassette, comprises upstream, i.e. at the 5′ end of the encoding sequence, a promoter and downstream, i.e. at the 3′ end, a polyadenylation signal and optionally other regulatory elements which are operably linked to the intervening encoding sequence with one of the nucleic acids as depicted in the respective line in Table I, application no. 4, column 5 or 8, preferably the coding region thereof, or a homolog or a fragment thereof. By an operable linkage is meant the sequential arrangement of promoter, encoding sequence, terminator, optionally other regulatory elements and optionally targeting sequences in such a way that each of the regulatory elements/targeting sequence can fulfill its function in the expression of the encoding sequence in due manner. In one embodiment the sequences preferred for operable linkage are targeting sequences for ensuring subcellular localization in organelles, like plastids or mitochondria. However, targeting sequences for ensuring subcellular localization in the endoplasmic reticulum (=ER), in the nucleus, in oil corpuscles or other compartments may also be employed as well as translation promoters such as the 5′ lead sequence in tobacco mosaic virus (Gallie et al., Nucl. Acids Res. 15, 8693 (1987)).

for the disclosure of this paragraph see [0157.1.1.1] above.

In an embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 4, column 5 or 8, preferably shown in Table II A, application no. 4, column 5, or in Table II A, application no. 4, column 8, or in Table II B, application no. 4, column 8, or a homolog or a fragment thereof;
   (b) a nucleic acid molecule shown in Table I, application no. 4, column 5 or 8, preferably shown in Table I A, application no. 4, column 5, or in Table I A, application no. 4, column 8, or in Table I B, application no. 4, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 4, preferably shown in Table II A, application no. 4, column 5, or in Table II A, application no. 4, column 8, or in Table II B, application no. 4, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nu oleic acid molecule sequence of a polynucleotide comprising the nucleic acid molecule shown in column 5 or 8 of Table I, application no. 4, preferably shown in Table I A, application no. 4, column 5, or in Table I A, application no. 4, column 8, or in Table I B, application no. 4, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 4;
   (i) a nucleic acid molecule encoding a polypeptide having the activity represented by a respective protein as depicted in column 5 of Table II, application no. 4;
   (j) a nucleic acid molecule comprising a polynucleotide which is obtained by amplifying a cDNA library or a genomic library using the primers in Table III, application no. 4, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto. and, optionally
3) nucleic acid molecule encoding a transit peptide, preferably encoding a plastidal transit peptide or a mitochondrial transit peptide;
   which are operable linked.
   (All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 4.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 4 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 4, in the same line as of the respective nucleic acid molecule according to 2) “plastidic” is depicted, or encoding a mitochondrial transit peptide, especially in case in column 6 of Table I, application no. 4, in the same line as of the respective nucleic acid molecule according to 2) “mitochondrial” is depicted.

In another embodiment thereof the expression cassette does not comprise 3) a nucleic acid molecule encoding a transit peptide, especially in case in column 6 of Table I, application no. 4, in the same line as of the respective nucleic acid molecule according to 2) “non-targeted” is depicted.

In a further embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 4, column 5 or 8, preferably shown in Table II A, application no. 4, column 5, or in Table II A, application no. 4, column 8, or in Table II B, application no. 4, column 8, or a homolog or a fragment thereof;
   (b) a nucleic acid molecule shown in Table I, application no. 4, column 5 or 8, preferably shown in Table I A, application no. 4, column 5, or in Table I A, application no. 4, column 8, or in Table I B, application no. 4, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 4, preferably shown in Table II A, application no. 4, column 5, or in Table II A, application no. 4, column 8, or in Table II B, application no. 4, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 4, preferably shown in Table I A, application no. 4, column 5, or in Table I A, application no. 4, column 8,or in Table I B, application no. 4, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 4;
   (i) a nucleic acid molecule encoding a polypeptide having the activity represented by a respective protein as depicted in column 5 of Table II, application no. 4;
   (j) a nucleic acid molecule comprising a polynucleotide which is obtained by amplifying a cDNA library or a genomic library using the primers in Table III, application no. 4, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto; and, optionally 3) nucleic acid molecule encoding a transit peptide, preferably encoding a plastidal transit peptide or a mitochondrial transit peptide;
      which are operable linked and
      whereby, preferably, the nucleic acid molecule according to 2 (a), (b), (c), (d), (e), (f), (g), (h), (i), (j) or (k) distinguishes over the sequence depicted in Table I A and/or I B, application no. 4, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention according to 2) does not consist of the sequence shown in Table I A and/or I B, application no. 4, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 4, column 5 or 8, or the coding region thereof, respectively. In a further embodiment the nucleic acid molecule does not encode the polypeptide sequence shown in Table II A and/or II B, application no. 4, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 4, column 5 or 8 and does not encode a protein of the sequence shown in Table II A and/or II B, application no. 4, column 5 or 8. Accordingly, in one embodiment, the protein encoded by a sequence of a nucleic acid according to 2(a), (b), (c), (d), (e), (f), (g), (h), (i), (j) or (k) does not consist of the sequence shown in Table I A and/or I B, application no. 4, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 4, column 5 or 8 but less than 100%, 99.999%, 99.99%, 99.9%, 99%, 98%, 97%, 96% or 95% identical to the sequence shown in Table II A and/or II B, application no. 4, column 5 or 8.
      (All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 4.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 4 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 4, in the same line as of the respective nucleic acid molecule according to 2) “plastidic” is depicted, or encoding a mitochondrial transit peptide, especially in case in column 6 of Table I, application no. 4, in the same line as of the respective nucleic acid molecule according to 2) “mitochondrial” is depicted.

In another embodiment thereof the expression cassette does not comprise 3) a nucleic acid molecule encoding a transit peptide, especially in case in column 6 of Table I, application no. 4, in the same line as of the respective nucleic acid molecule according to 2) “non-targeted” is depicted.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 15187, or a homolog or fragment thereof, or a homolog thereof being depicted in Table I, application no. 4, column 8, in the same line as SEQ ID NO. 15187, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 15187 is depicted, plastidic is mentioned, or a mitochondrial transit peptide in case in Table I, application no. 4, in column 6 in the same line as SEQ ID NO. 15187 is depicted, mitochondrial is mentioned; which is operable linked, too.

In another embodiment thereof the expression cassette does not comprise an additional transit peptide in case in Table I, application no. 4, in column 6 in the same line as SEQ ID NO. 15187 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 69, or a homolog or fragment thereof, or a homolog thereof being depicted in Table I, application no. 4, column 8, in the same line as SEQ ID NO. 69, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 69 is depicted, plastidic is mentioned, or a mitochondrial transit peptide in case in Table I, application no. 4, in column 6 in the same line as SEQ ID NO. 69 is depicted, mitochondrial is mentioned; which is operable linked, too.

In another embodiment thereof the expression cassette does not comprise an additional transit peptide in case in Table I, application no. 4, in column 6 in the same line as SEQ ID NO. 69 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 16883, or a homolog or fragment thereof, or a homolog thereof being depicted in Table I, application no. 4, column 8, in the same line as SEQ ID NO. 16883, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 16883 is depicted, plastidic is mentioned, or a mitochondrial transit peptide in case in Table I, application no. 4, in column 6 in the same line as SEQ ID NO. 16883 is depicted, mitochondrial is mentioned; which is operable linked, too.

In another embodiment thereof the expression cassette does not comprise an additional transit peptide in case in Table I, application no. 4, in column 6 in the same line as SEQ ID NO. 16883 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 352, or a homolog or fragment thereof, or a homolog thereof being depicted in Table I, application no. 4, column 8, in the same line as SEQ ID NO. 352, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 352 is depicted, plastidic is mentioned, or a mitochondrial transit peptide in case in Table I, application no. 4, in column 6 in the same line as SEQ ID NO. 352 is depicted, mitochondrial is mentioned; which is operable linked, too.

In another embodiment thereof the expression cassette does not comprise an additional transit peptide in case in Table I, application no. 4, in column 6 in the same line as SEQ ID NO. 352 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 627, or a homolog or fragment thereof, or a homolog thereof being depicted in Table I, application no. 4, column 8, in the same line as SEQ ID NO. 627, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 627 is depicted, plastidic is mentioned, or a mitochondrial transit peptide in case in Table I, application no. 4, in column 6 in the same line as SEQ ID NO. 627 is depicted, mitochondrial is mentioned; which is operable linked, too.

In another embodiment thereof the expression cassette does not comprise an additional transit peptide in case in Table I, application no. 4, in column 6 in the same line as SEQ ID NO. 627 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17968, or a homolog or fragment thereof, or a homolog thereof being depicted in Table I, application no. 4, column 8, in the same line as SEQ ID NO. 17968, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 17968 is depicted, plastidic is mentioned, or a mitochondrial transit peptide in case in Table I, application no. 4, in column 6 in the same line as SEQ ID NO. 17968 is depicted, mitochondrial is mentioned; which is operable linked, too.

In another embodiment thereof the expression cassette does not comprise an additional transit peptide in case in Table I, application no. 4, in column 6 in the same line as SEQ ID NO. 17968 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 18070, or a homolog or fragment thereof, or a homolog thereof being depicted in Table I, application no. 4, column 8, in the same line as SEQ ID NO. 18070, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 18070 is depicted, plastidic is mentioned, or a mitochondrial transit peptide in case in Table I, application no. 4, in column 6 in the same line as SEQ ID NO. 18070 is depicted, mitochondrial is mentioned; which is operable linked, too.

In another embodiment thereof the expression cassette does not comprise an additional transit peptide in case in Table I, application no. 4, in column 6 in the same line as SEQ ID NO. 18070 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 812, or a homolog or fragment thereof, or a homolog thereof being depicted in Table I, application no. 4, column 8, in the same line as SEQ ID NO. 812, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 812 is depicted, plastidic is mentioned, or a mitochondrial transit peptide in case in Table I, application no. 4, in column 6 in the same line as SEQ ID NO. 812 is depicted, mitochondrial is mentioned; which is operable linked, too.

In another embodiment thereof the expression cassette does not comprise an additional transit peptide in case in Table I, application no. 4, in column 6 in the same line as SEQ ID NO. 812 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 18235, or a homolog or fragment thereof, or a homolog thereof being depicted in Table I, application no. 4, column 8, in the same line as SEQ ID NO. 18235, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 18235 is depicted, plastidic is mentioned, or a mitochondrial transit peptide in case in Table I, application no. 4, in column 6 in the same line as SEQ ID NO. 18235 is depicted, mitochondrial is mentioned; which is operable linked, too.

In another embodiment thereof the expression cassette does not comprise an additional transit peptide in case in Table I, application no. 4, in column 6 in the same line as SEQ ID NO. 18235 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 19419, or a homolog or fragment thereof, or a homolog thereof being depicted in Table I, application no. 4, column 8, in the same line as SEQ ID NO. 19419, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 19419 is depicted, plastidic is mentioned, or a mitochondrial transit peptide in case in Table I, application no. 4, in column 6 in the same line as SEQ ID NO. 19419 is depicted, mitochondrial is mentioned; which is operable linked, too.

In another embodiment thereof the expression cassette does not comprise an additional transit peptide in case in Table I, application no. 4, in column 6 in the same line as SEQ ID NO. 19419 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 1298, or a homolog or fragment thereof, or a homolog thereof being depicted in Table I, application no. 4, column 8, in the same line as SEQ ID NO. 1298, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 1298 is depicted, plastidic is mentioned, or a mitochondrial transit peptide in case in Table I, application no. 4, in column 6 in the same line as SEQ ID NO. 1298 is depicted, mitochondrial is mentioned; which is operable linked, too.

In another embodiment thereof the expression cassette does not comprise an additional transit peptide in case in Table I, application no. 4, in column 6 in the same line as SEQ ID NO. 1298 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 20346, or a homolog or fragment thereof, or a homolog thereof being depicted in Table I, application no. 4, column 8, in the same line as SEQ ID NO. 20346, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 20346 is depicted, plastidic is mentioned, or a mitochondrial transit peptide in case in Table I, application no. 4, in column 6 in the same line as SEQ ID NO. 20346 is depicted, mitochondrial is mentioned; which is operable linked, too.

In another embodiment thereof the expression cassette does not comprise an additional transit peptide in case in Table I, application no. 4, in column 6 in the same line as SEQ ID NO. 20346 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 22921, or a homolog or fragment thereof, or a homolog thereof being depicted in Table I, application no. 4, column 8, in the same line as SEQ ID NO. 22921, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 22921 is depicted, plastidic is mentioned, or a mitochondrial transit peptide in case in Table I, application no. 4, in column 6 in the same line as SEQ ID NO. 22921 is depicted, mitochondrial is mentioned; which is operable linked, too.

In another embodiment thereof the expression cassette does not comprise an additional transit peptide in case in Table I, application no. 4, in column 6 in the same line as SEQ ID NO. 22921 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 1815, or a homolog or fragment thereof, or a homolog thereof being depicted in Table I, application no. 4, column 8, in the same line as SEQ ID NO. 1815, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 1815 is depicted, plastidic is mentioned, or a mitochondrial transit peptide in case in Table I, application no. 4, in column 6 in the same line as SEQ ID NO. 1815 is depicted, mitochondrial is mentioned; which is operable linked, too.

In another embodiment thereof the expression cassette does not comprise an additional transit peptide in case in Table I, application no. 4, in column 6 in the same line as SEQ ID NO. 1815 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68777, or a homolog or fragment thereof, or a homolog thereof being depicted in Table I, application no. 4, column 8, in the same line as SEQ ID NO. 68777, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 68777 is depicted, plastidic is mentioned, or a mitochondrial transit peptide in case in Table I, application no. 4, in column 6 in the same line as SEQ ID NO. 68777 is depicted, mitochondrial is mentioned; which is operable linked, too.

In another embodiment thereof the expression cassette does not comprise an additional transit peptide in case in Table I, application no. 4, in column 6 in the same line as SEQ ID NO. 68777 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 2573, or a homolog or fragment thereof, or a homolog thereof being depicted in Table I, application no. 4, column 8, in the same line as SEQ ID NO. 2573, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 2573 is depicted, plastidic is mentioned, or a mitochondrial transit peptide in case in Table I, application no. 4, in column 6 in the same line as SEQ ID NO. 2573 is depicted, mitochondrial is mentioned; which is operable linked, too.

In another embodiment thereof the expression cassette does not comprise an additional transit peptide in case in Table I, application no. 4, in column 6 in the same line as SEQ ID NO. 2573 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 2935, or a homolog or fragment thereof, or a homolog thereof being depicted in Table I, application no. 4, column 8, in the same line as SEQ ID NO. 2935, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 2935 is depicted, plastidic is mentioned, or a mitochondrial transit peptide in case in Table I, application no. 4, in column 6 in the same line as SEQ ID NO. 2935 is depicted, mitochondrial is mentioned; which is operable linked, too.

In another embodiment thereof the expression cassette does not comprise an additional transit peptide in case in Table I, application no. 4, in column 6 in the same line as SEQ ID NO. 2935 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 23482, or a homolog or fragment thereof, or a homolog thereof being depicted in Table I, application no. 4, column 8, in the same line as SEQ ID NO. 23482, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 23482 is depicted, plastidic is mentioned, or a mitochondrial transit peptide in case in Table I, application no. 4, in column 6 in the same line as SEQ ID NO. 23482 is depicted, mitochondrial is mentioned; which is operable linked, too.

In another embodiment thereof the expression cassette does not comprise an additional transit peptide in case in Table I, application no. 4, in column 6 in the same line as SEQ ID NO. 23482 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 3279, or a homolog or fragment thereof, or a homolog thereof being depicted in Table I, application no. 4, column 8, in the same line as SEQ ID NO. 3279, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 3279 is depicted, plastidic is mentioned, or a mitochondrial transit peptide in case in Table I, application no. 4, in column 6 in the same line as SEQ ID NO. 3279 is depicted, mitochondrial is mentioned; which is operable linked, too.

In another embodiment thereof the expression cassette does not comprise an additional transit peptide in case in Table I, application no. 4, in column 6 in the same line as SEQ ID NO. 3279 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 23844, or a homolog or fragment thereof, or a homolog thereof being depicted in Table I, application no. 4, column 8, in the same line as SEQ ID NO. 23844, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 23844 is depicted, plastidic is mentioned, or a mitochondrial transit peptide in case in Table I, application no. 4, in column 6 in the same line as SEQ ID NO. 23844 is depicted, mitochondrial is mentioned; which is operable linked, too.

In another embodiment thereof the expression cassette does not comprise an additional transit peptide in case in Table I, application no. 4, in column 6 in the same line as SEQ ID NO. 23844 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 24232, or a homolog or fragment thereof, or a homolog thereof being depicted in Table I, application no. 4, column 8, in the same line as SEQ ID NO. 24232, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 24232 is depicted, plastidic is mentioned, or a mitochondrial transit peptide in case in Table I, application no. 4, in column 6 in the same line as SEQ ID NO. 24232 is depicted, mitochondrial is mentioned; which is operable linked, too.

In another embodiment thereof the expression cassette does not comprise an additional transit peptide in case in Table I, application no. 4, in column 6 in the same line as SEQ ID NO. 24232 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4040, or a homolog or fragment thereof, or a homolog thereof being depicted in Table I, application no. 4, column 8, in the same line as SEQ ID NO. 4040, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 4040 is depicted, plastidic is mentioned, or a mitochondrial transit peptide in case in Table I, application no. 4, in column 6 in the same line as SEQ ID NO. 4040 is depicted, mitochondrial is mentioned; which is operable linked, too.

In another embodiment thereof the expression cassette does not comprise an additional transit peptide in case in Table I, application no. 4, in column 6 in the same line as SEQ ID NO. 4040 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 24311, or a homolog or fragment thereof, or a homolog thereof being depicted in Table I, application no. 4, column 8, in the same line as SEQ ID NO. 24311, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 24311 is depicted, plastidic is mentioned, or a mitochondrial transit peptide in case in Table I, application no. 4, in column 6 in the same line as SEQ ID NO. 24311 is depicted, mitochondrial is mentioned; which is operable linked, too.

In another embodiment thereof the expression cassette does not comprise an additional transit peptide in case in Table I, application no. 4, in column 6 in the same line as SEQ ID NO. 24311 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4348, or a homolog or fragment thereof, or a homolog thereof being depicted in Table I, application no. 4, column 8, in the same line as SEQ ID NO. 4348, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 4348 is depicted, plastidic is mentioned, or a mitochondrial transit peptide in case in Table I, application no. 4, in column 6 in the same line as SEQ ID NO. 4348 is depicted, mitochondrial is mentioned; which is operable linked, too.

In another embodiment thereof the expression cassette does not comprise an additional transit peptide in case in Table I, application no. 4, in column 6 in the same line as SEQ ID NO. 4348 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 24492, or a homolog or fragment thereof, or a homolog thereof being depicted in Table I, application no. 4, column 8, in the same line as SEQ ID NO. 24492, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 24492 is depicted, plastidic is mentioned, or a mitochondrial transit peptide in case in Table I, application no. 4, in column 6 in the same line as SEQ ID NO. 24492 is depicted, mitochondrial is mentioned; which is operable linked, too.

In another embodiment thereof the expression cassette does not comprise an additional transit peptide in case in Table I, application no. 4, in column 6 in the same line as SEQ ID NO. 24492 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 93451, or a homolog or fragment thereof, or a homolog thereof being depicted in Table I, application no. 4, column 8, in the same line as SEQ ID NO. 93451, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 93451 is depicted, plastidic is mentioned, or a mitochondrial transit peptide in case in Table I, application no. 4, in column 6 in the same line as SEQ ID NO. 93451 is depicted, mitochondrial is mentioned; which is operable linked, too.

In another embodiment thereof the expression cassette does not comprise an additional transit peptide in case in Table I, application no. 4, in column 6 in the same line as SEQ ID NO. 93451 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 25283, or a homolog or fragment thereof, or a homolog thereof being depicted in Table I, application no. 4, column 8, in the same line as SEQ ID NO. 25283, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 25283 is depicted, plastidic is mentioned, or a mitochondrial transit peptide in case in Table I, application no. 4, in column 6 in the same line as SEQ ID NO. 25283 is depicted, mitochondrial is mentioned; which is operable linked, too.

In another embodiment thereof the expression cassette does not comprise an additional transit peptide in case in Table I, application no. 4, in column 6 in the same line as SEQ ID NO. 25283 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 69574, or a homolog or fragment thereof, or a homolog thereof being depicted in Table I, application no. 4, column 8, in the same line as SEQ ID NO. 69574, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 69574 is depicted, plastidic is mentioned, or a mitochondrial transit peptide in case in Table I, application no. 4, in column 6 in the same line as SEQ ID NO. 69574 is depicted, mitochondrial is mentioned; which is operable linked, too.

In another embodiment thereof the expression cassette does not comprise an additional transit peptide in case in Table I, application no. 4, in column 6 in the same line as SEQ ID NO. 69574 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 5318, or a homolog or fragment thereof, or a homolog thereof being depicted in Table I, application no. 4, column 8, in the same line as SEQ ID NO. 5318, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 5318 is depicted, plastidic is mentioned, or a mitochondrial transit peptide in case in Table I, application no. 4, in column 6 in the same line as SEQ ID NO. 5318 is depicted, mitochondrial is mentioned; which is operable linked, too.

In another embodiment thereof the expression cassette does not comprise an additional transit peptide in case in Table I, application no. 4, in column 6 in the same line as SEQ ID NO. 5318 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 5493, or a homolog or fragment thereof, or a homolog thereof being depicted in Table I, application no. 4, column 8, in the same line as SEQ ID NO. 5493, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 5493 is depicted, plastidic is mentioned, or a mitochondrial transit peptide in case in Table I, application no. 4, in column 6 in the same line as SEQ ID NO. 5493 is depicted, mitochondrial is mentioned; which is operable linked, too.

In another embodiment thereof the expression cassette does not comprise an additional transit peptide in case in Table I, application no. 4, in column 6 in the same line as SEQ ID NO. 5493 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 70583, or a homolog or fragment thereof, or a homolog thereof being depicted in Table I, application no. 4, column 8, in the same line as SEQ ID NO. 70583, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 70583 is depicted, plastidic is mentioned, or a mitochondrial transit peptide in case in Table I, application no. 4, in column 6 in the same line as SEQ ID NO. 70583 is depicted, mitochondrial is mentioned; which is operable linked, too.

In another embodiment thereof the expression cassette does not comprise an additional transit peptide in case in Table I, application no. 4, in column 6 in the same line as SEQ ID NO. 70583 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 5557, or a homolog or fragment thereof, or a homolog thereof being depicted in Table I, application no. 4, column 8, in the same line as SEQ ID NO. 5557, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 5557 is depicted, plastidic is mentioned, or a mitochondrial transit peptide in case in Table I, application no. 4, in column 6 in the same line as SEQ ID NO. 5557 is depicted, mitochondrial is mentioned; which is operable linked, too.

In another embodiment thereof the expression cassette does not comprise an additional transit peptide in case in Table I, application no. 4, in column 6 in the same line as SEQ ID NO. 5557 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 26196, or a homolog or fragment thereof, or a homolog thereof being depicted in Table I, application no. 4, column 8, in the same line as SEQ ID NO. 26196, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 26196 is depicted, plastidic is mentioned, or a mitochondrial transit peptide in case in Table I, application no. 4, in column 6 in the same line as SEQ ID NO. 26196 is depicted, mitochondrial is mentioned; which is operable linked, too.

In another embodiment thereof the expression cassette does not comprise an additional transit peptide in case in Table I, application no. 4, in column 6 in the same line as SEQ ID NO. 26196 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6040, or a homolog or fragment thereof, or a homolog thereof being depicted in Table I, application no. 4, column 8, in the same line as SEQ ID NO. 6040, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 6040 is depicted, plastidic is mentioned, or a mitochondrial transit peptide in case in Table I, application no. 4, in column 6 in the same line as SEQ ID NO. 6040 is depicted, mitochondrial is mentioned; which is operable linked, too.

In another embodiment thereof the expression cassette does not comprise an additional transit peptide in case in Table I, application no. 4, in column 6 in the same line as SEQ ID NO. 6040 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 27021, or a homolog or fragment thereof, or a homolog thereof being depicted in Table I, application no. 4, column 8, in the same line as SEQ ID NO. 27021, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 27021 is depicted, plastidic is mentioned, or a mitochondrial transit peptide in case in Table I, application no. 4, in column 6 in the same line as SEQ ID NO. 27021 is depicted, mitochondrial is mentioned; which is operable linked, too.

In another embodiment thereof the expression cassette does not comprise an additional transit peptide in case in Table I, application no. 4, in column 6 in the same line as SEQ ID NO. 27021 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 27882, or a homolog or fragment thereof, or a homolog thereof being depicted in Table I, application no. 4, column 8, in the same line as SEQ ID NO. 27882, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 27882 is depicted, plastidic is mentioned, or a mitochondrial transit peptide in case in Table I, application no. 4, in column 6 in the same line as SEQ ID NO. 27882 is depicted, mitochondrial is mentioned; which is operable linked, too.

In another embodiment thereof the expression cassette does not comprise an additional transit peptide in case in Table I, application no. 4, in column 6 in the same line as SEQ ID NO. 27882 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 28040, or a homolog or fragment thereof, or a homolog thereof being depicted in Table I, application no. 4, column 8, in the same line as SEQ ID NO. 28040, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 28040 is depicted, plastidic is mentioned, or a mitochondrial transit peptide in case in Table I, application no. 4, in column 6 in the same line as SEQ ID NO. 28040 is depicted, mitochondrial is mentioned; which is operable linked, too.

In another embodiment thereof the expression cassette does not comprise an additional transit peptide in case in Table I, application no. 4, in column 6 in the same line as SEQ ID NO. 28040 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6075, or a homolog or fragment thereof, or a homolog thereof being depicted in Table I, application no. 4, column 8, in the same line as SEQ ID NO. 6075, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 6075 is depicted, plastidic is mentioned, or a mitochondrial transit peptide in case in Table I, application no. 4, in column 6 in the same line as SEQ ID NO. 6075 is depicted, mitochondrial is mentioned; which is operable linked, too.

In another embodiment thereof the expression cassette does not comprise an additional transit peptide in case in Table I, application no. 4, in column 6 in the same line as SEQ ID NO. 6075 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 93532, or a homolog or fragment thereof, or a homolog thereof being depicted in Table I, application no. 4, column 8, in the same line as SEQ ID NO. 93532, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 93532 is depicted, plastidic is mentioned, or a mitochondrial transit peptide in case in Table I, application no. 4, in column 6 in the same line as SEQ ID NO. 93532 is depicted, mitochondrial is mentioned; which is operable linked, too.

In another embodiment thereof the expression cassette does not comprise an additional transit peptide in case in Table I, application no. 4, in column 6 in the same line as SEQ ID NO. 93532 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 72346, or a homolog or fragment thereof, or a homolog thereof being depicted in Table I, application no. 4, column 8, in the same line as SEQ ID NO. 72346, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 72346 is depicted, plastidic is mentioned, or a mitochondrial transit peptide in case in Table I, application no. 4, in column 6 in the same line as SEQ ID NO. 72346 is depicted, mitochondrial is mentioned; which is operable linked, too.

In another embodiment thereof the expression cassette does not comprise an additional transit peptide in case in Table I, application no. 4, in column 6 in the same line as SEQ ID NO. 72346 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 28738, or a homolog or fragment thereof, or a homolog thereof being depicted in Table I, application no. 4, column 8, in the same line as SEQ ID NO. 28738, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 28738 is depicted, plastidic is mentioned, or a mitochondrial transit peptide in case in Table I, application no. 4, in column 6 in the same line as SEQ ID NO. 28738 is depicted, mitochondrial is mentioned; which is operable linked, too.

In another embodiment thereof the expression cassette does not comprise an additional transit peptide in case in Table I, application no. 4, in column 6 in the same line as SEQ ID NO. 28738 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 29286, or a homolog or fragment thereof, or a homolog thereof being depicted in Table I, application no. 4, column 8, in the same line as SEQ ID NO. 29286, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 29286 is depicted, plastidic is mentioned, or a mitochondrial transit peptide in case in Table I, application no. 4, in column 6 in the same line as SEQ ID NO. 29286 is depicted, mitochondrial is mentioned; which is operable linked, too.

In another embodiment thereof the expression cassette does not comprise an additional transit peptide in case in Table I, application no. 4, in column 6 in the same line as SEQ ID NO. 29286 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6268, or a homolog or fragment thereof, or a homolog thereof being depicted in Table I, application no. 4, column 8, in the same line as SEQ ID NO. 6268, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 6268 is depicted, plastidic is mentioned, or a mitochondrial transit peptide in case in Table I, application no. 4, in column 6 in the same line as SEQ ID NO. 6268 is depicted, mitochondrial is mentioned; which is operable linked, too.

In another embodiment thereof the expression cassette does not comprise an additional transit peptide in case in Table I, application no. 4, in column 6 in the same line as SEQ ID NO. 6268 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6510, or a homolog or fragment thereof, or a homolog thereof being depicted in Table I, application no. 4, column 8, in the same line as SEQ ID NO. 6510, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 6510 is depicted, plastidic is mentioned, or a mitochondrial transit peptide in case in Table I, application no. 4, in column 6 in the same line as SEQ ID NO. 6510 is depicted, mitochondrial is mentioned; which is operable linked, too.

In another embodiment thereof the expression cassette does not comprise an additional transit peptide in case in Table I, application no. 4, in column 6 in the same line as SEQ ID NO. 6510 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 32037, or a homolog or fragment thereof, or a homolog thereof being depicted in Table I, application no. 4, column 8, in the same line as SEQ ID NO. 32037, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 32037 is depicted, plastidic is mentioned, or a mitochondrial transit peptide in case in Table I, application no. 4, in column 6 in the same line as SEQ ID NO. 32037 is depicted, mitochondrial is mentioned; which is operable linked, too.

In another embodiment thereof the expression cassette does not comprise an additional transit peptide in case in Table I, application no. 4, in column 6 in the same line as SEQ ID NO. 32037 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 74662, or a homolog or fragment thereof, or a homolog thereof being depicted in Table I, application no. 4, column 8, in the same line as SEQ ID NO. 74662, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 74662 is depicted, plastidic is mentioned, or a mitochondrial transit peptide in case in Table I, application no. 4, in column 6 in the same line as SEQ ID NO. 74662 is depicted, mitochondrial is mentioned; which is operable linked, too.

In another embodiment thereof the expression cassette does not comprise an additional transit peptide in case in Table I, application no. 4, in column 6 in the same line as SEQ ID NO. 74662 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 33596, or a homolog or fragment thereof, or a homolog thereof being depicted in Table I, application no. 4, column 8, in the same line as SEQ ID NO. 33596, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 33596 is depicted, plastidic is mentioned, or a mitochondrial transit peptide in case in Table I, application no. 4, in column 6 in the same line as SEQ ID NO. 33596 is depicted, mitochondrial is mentioned; which is operable linked, too.

In another embodiment thereof the expression cassette does not comprise an additional transit peptide in case in Table I, application no. 4, in column 6 in the same line as SEQ ID NO. 33596 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 34204, or a homolog or fragment thereof, or a homolog thereof being depicted in Table I, application no. 4, column 8, in the same line as SEQ ID NO. 34204, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 34204 is depicted, plastidic is mentioned, or a mitochondrial transit peptide in case in Table I, application no. 4, in column 6 in the same line as SEQ ID NO. 34204 is depicted, mitochondrial is mentioned; which is operable linked, too.

In another embodiment thereof the expression cassette does not comprise an additional transit peptide in case in Table I, application no. 4, in column 6 in the same line as SEQ ID NO. 34204 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 34301, or a homolog or fragment thereof, or a homolog thereof being depicted in Table I, application no. 4, column 8, in the same line as SEQ ID NO. 34301, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 34301 is depicted, plastidic is mentioned, or a mitochondrial transit peptide in case in Table I, application no. 4, in column 6 in the same line as SEQ ID NO. 34301 is depicted, mitochondrial is mentioned; which is operable linked, too.

In another embodiment thereof the expression cassette does not comprise an additional transit peptide in case in Table I, application no. 4, in column 6 in the same line as SEQ ID NO. 34301 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 34889, or a homolog or fragment thereof, or a homolog thereof being depicted in Table I, application no. 4, column 8, in the same line as SEQ ID NO. 34889, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 34889 is depicted, plastidic is mentioned, or a mitochondrial transit peptide in case in Table I, application no. 4, in column 6 in the same line as SEQ ID NO. 34889 is depicted, mitochondrial is mentioned; which is operable linked, too.

In another embodiment thereof the expression cassette does not comprise an additional transit peptide in case in Table I, application no. 4, in column 6 in the same line as SEQ ID NO. 34889 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 6818, or a homolog or fragment thereof, or a homolog thereof being depicted in Table I, application no. 4, column 8, in the same line as SEQ ID NO. 6818, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 6818 is depicted, plastidic is mentioned, or a mitochondrial transit peptide in case in Table I, application no. 4, in column 6 in the same line as SEQ ID NO. 6818 is depicted, mitochondrial is mentioned; which is operable linked, too.

In another embodiment thereof the expression cassette does not comprise an additional transit peptide in case in Table I, application no. 4, in column 6 in the same line as SEQ ID NO. 6818 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 35366, or a homolog or fragment thereof, or a homolog thereof being depicted in Table I, application no. 4, column 8, in the same line as SEQ ID NO. 35366, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 35366 is depicted, plastidic is mentioned, or a mitochondrial transit peptide in case in Table I, application no. 4, in column 6 in the same line as SEQ ID NO. 35366 is depicted, mitochondrial is mentioned; which is operable linked, too.

In another embodiment thereof the expression cassette does not comprise an additional transit peptide in case in Table I, application no. 4, in column 6 in the same line as SEQ ID NO. 35366 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 35482, or a homolog or fragment thereof, or a homolog thereof being depicted in Table I, application no. 4, column 8, in the same line as SEQ ID NO. 35482, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 35482 is depicted, plastidic is mentioned, or a mitochondrial transit peptide in case in Table I, application no. 4, in column 6 in the same line as SEQ ID NO. 35482 is depicted, mitochondrial is mentioned; which is operable linked, too.

In another embodiment thereof the expression cassette does not comprise an additional transit peptide in case in Table I, application no. 4, in column 6 in the same line as SEQ ID NO. 35482 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7081, or a homolog or fragment thereof, or a homolog thereof being depicted in Table I, application no. 4, column 8, in the same line as SEQ ID NO. 7081, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 7081 is depicted, plastidic is mentioned, or a mitochondrial transit peptide in case in Table I, application no. 4, in column 6 in the same line as SEQ ID NO. 7081 is depicted, mitochondrial is mentioned; which is operable linked, too.

In another embodiment thereof the expression cassette does not comprise an additional transit peptide in case in Table I, application no. 4, in column 6 in the same line as SEQ ID NO. 7081 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 35590, or a homolog or fragment thereof, or a homolog thereof being depicted in Table I, application no. 4, column 8, in the same line as SEQ ID NO. 35590, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 35590 is depicted, plastidic is mentioned, or a mitochondrial transit peptide in case in Table I, application no. 4, in column 6 in the same line as SEQ ID NO. 35590 is depicted, mitochondrial is mentioned; which is operable linked, too.

In another embodiment thereof the expression cassette does not comprise an additional transit peptide in case in Table I, application no. 4, in column 6 in the same line as SEQ ID NO. 35590 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 35733, or a homolog or fragment thereof, or a homolog thereof being depicted in Table I, application no. 4, column 8, in the same line as SEQ ID NO. 35733, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 35733 is depicted, plastidic is mentioned, or a mitochondrial transit peptide in case in Table I, application no. 4, in column 6 in the same line as SEQ ID NO. 35733 is depicted, mitochondrial is mentioned; which is operable linked, too.

In another embodiment thereof the expression cassette does not comprise an additional transit peptide in case in Table I, application no. 4, in column 6 in the same line as SEQ ID NO. 35733 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 7269, or a homolog or fragment thereof, or a homolog thereof being depicted in Table I, application no. 4, column 8, in the same line as SEQ ID NO. 7269, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 7269 is depicted, plastidic is mentioned, or a mitochondrial transit peptide in case in Table I, application no. 4, in column 6 in the same line as SEQ ID NO. 7269 is depicted, mitochondrial is mentioned; which is operable linked, too.

In another embodiment thereof the expression cassette does not comprise an additional transit peptide in case in Table I, application no. 4, in column 6 in the same line as SEQ ID NO. 7269 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7333, or a homolog or fragment thereof, or a homolog thereof being depicted in Table I, application no. 4, column 8, in the same line as SEQ ID NO. 7333, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 7333 is depicted, plastidic is mentioned, or a mitochondrial transit peptide in case in Table I, application no. 4, in column 6 in the same line as SEQ ID NO. 7333 is depicted, mitochondrial is mentioned; which is operable linked, too.

In another embodiment thereof the expression cassette does not comprise an additional transit peptide in case in Table I, application no. 4, in column 6 in the same line as SEQ ID NO. 7333 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7686, or a homolog or fragment thereof, or a homolog thereof being depicted in Table I, application no. 4, column 8, in the same line as SEQ ID NO. 7686, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 7686 is depicted, plastidic is mentioned, or a mitochondrial transit peptide in case in Table I, application no. 4, in column 6 in the same line as SEQ ID NO. 7686 is depicted, mitochondrial is mentioned; which is operable linked, too.

In another embodiment thereof the expression cassette does not comprise an additional transit peptide in case in Table I, application no. 4, in column 6 in the same line as SEQ ID NO. 7686 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 35967, or a homolog or fragment thereof, or a homolog thereof being depicted in Table I, application no. 4, column 8, in the same line as SEQ ID NO. 35967, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 35967 is depicted, plastidic is mentioned, or a mitochondrial transit peptide in case in Table I, application no. 4, in column 6 in the same line as SEQ ID NO. 35967 is depicted, mitochondrial is mentioned; which is operable linked, too.

In another embodiment thereof the expression cassette does not comprise an additional transit peptide in case in Table I, application no. 4, in column 6 in the same line as SEQ ID NO. 35967 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 93678, or a homolog or fragment thereof, or a homolog thereof being depicted in Table I, application no. 4, column 8, in the same line as SEQ ID NO. 93678, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 93678 is depicted, plastidic is mentioned, or a mitochondrial transit peptide in case in Table I, application no. 4, in column 6 in the same line as SEQ ID NO. 93678 is depicted, mitochondrial is mentioned; which is operable linked, too.

In another embodiment thereof the expression cassette does not comprise an additional transit peptide in case in Table I, application no. 4, in column 6 in the same line as SEQ ID NO. 93678 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 93793, or a homolog or fragment thereof, or a homolog thereof being depicted in Table I, application no. 4, column 8, in the same line as SEQ ID NO. 93793, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 93793 is depicted, plastidic is mentioned, or a mitochondrial transit peptide in case in Table I, application no. 4, in column 6 in the same line as SEQ ID NO. 93793 is depicted, mitochondrial is mentioned; which is operable linked, too.

In another embodiment thereof the expression cassette does not comprise an additional transit peptide in case in Table I, application no. 4, in column 6 in the same line as SEQ ID NO. 93793 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7917, or a homolog or fragment thereof, or a homolog thereof being depicted in Table I, application no. 4, column 8, in the same line as SEQ ID NO. 7917, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 7917 is depicted, plastidic is mentioned, or a mitochondrial transit peptide in case in Table I, application no. 4, in column 6 in the same line as SEQ ID NO. 7917 is depicted, mitochondrial is mentioned; which is operable linked, too.

In another embodiment thereof the expression cassette does not comprise an additional transit peptide in case in Table I, application no. 4, in column 6 in the same line as SEQ ID NO. 7917 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 36809, or a homolog or fragment thereof, or a homolog thereof being depicted in Table I, application no. 4, column 8, in the same line as SEQ ID NO. 36809, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 36809 is depicted, plastidic is mentioned, or a mitochondrial transit peptide in case in Table I, application no. 4, in column 6 in the same line as SEQ ID NO. 36809 is depicted, mitochondrial is mentioned; which is operable linked, too.

In another embodiment thereof the expression cassette does not comprise an additional transit peptide in case in Table I, application no. 4, in column 6 in the same line as SEQ ID NO. 36809 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7941, or a homolog or fragment thereof, or a homolog thereof being depicted in Table I, application no. 4, column 8, in the same line as SEQ ID NO. 7941, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 7941 is depicted, plastidic is mentioned, or a mitochondrial transit peptide in case in Table I, application no. 4, in column 6 in the same line as SEQ ID NO. 7941 is depicted, mitochondrial is mentioned; which is operable linked, too.

In another embodiment thereof the expression cassette does not comprise an additional transit peptide in case in Table I, application no. 4, in column 6 in the same line as SEQ ID NO. 7941 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 36937, or a homolog or fragment thereof, or a homolog thereof being depicted in Table I, application no. 4, column 8, in the same line as SEQ ID NO. 36937, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 36937 is depicted, plastidic is mentioned, or a mitochondrial transit peptide in case in Table I, application no. 4, in column 6 in the same line as SEQ ID NO. 36937 is depicted, mitochondrial is mentioned; which is operable linked, too.

In another embodiment thereof the expression cassette does not comprise an additional transit peptide in case in Table I, application no. 4, in column 6 in the same line as SEQ ID NO. 36937 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 77768, or a homolog or fragment thereof, or a homolog thereof being depicted in Table I, application no. 4, column 8, in the same line as SEQ ID NO. 77768, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 77768 is depicted, plastidic is mentioned, or a mitochondrial transit peptide in case in Table I, application no. 4, in column 6 in the same line as SEQ ID NO. 77768 is depicted, mitochondrial is mentioned; which is operable linked, too.

In another embodiment thereof the expression cassette does not comprise an additional transit peptide in case in Table I, application no. 4, in column 6 in the same line as SEQ ID NO. 77768 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38289, or a homolog or fragment thereof, or a homolog thereof being depicted in Table I, application no. 4, column 8, in the same line as SEQ ID NO. 38289, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 38289 is depicted, plastidic is mentioned, or a mitochondrial transit peptide in case in Table I, application no. 4, in column 6 in the same line as SEQ ID NO. 38289 is depicted, mitochondrial is mentioned; which is operable linked, too.

In another embodiment thereof the expression cassette does not comprise an additional transit peptide in case in Table I, application no. 4, in column 6 in the same line as SEQ ID NO. 38289 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 77774, or a homolog or fragment thereof, or a homolog thereof being depicted in Table I, application no. 4, column 8, in the same line as SEQ ID NO. 77774, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 77774 is depicted, plastidic is mentioned, or a mitochondrial transit peptide in case in Table I, application no. 4, in column 6 in the same line as SEQ ID NO. 77774 is depicted, mitochondrial is mentioned; which is operable linked, too.

In another embodiment thereof the expression cassette does not comprise an additional transit peptide in case in Table I, application no. 4, in column 6 in the same line as SEQ ID NO. 77774 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7947, or a homolog or fragment thereof, or a homolog thereof being depicted in Table I, application no. 4, column 8, in the same line as SEQ ID NO. 7947, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 7947 is depicted, plastidic is mentioned, or a mitochondrial transit peptide in case in Table I, application no. 4, in column 6 in the same line as SEQ ID NO. 7947 is depicted, mitochondrial is mentioned; which is operable linked, too.

In another embodiment thereof the expression cassette does not comprise an additional transit peptide in case in Table I, application no. 4, in column 6 in the same line as SEQ ID NO. 7947 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78753, or a homolog or fragment thereof, or a homolog thereof being depicted in Table I, application no. 4, column 8, in the same line as SEQ ID NO. 78753, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 78753 is depicted, plastidic is mentioned, or a mitochondrial transit peptide in case in Table I, application no. 4, in column 6 in the same line as SEQ ID NO. 78753 is depicted, mitochondrial is mentioned; which is operable linked, too.

In another embodiment thereof the expression cassette does not comprise an additional transit peptide in case in Table I, application no. 4, in column 6 in the same line as SEQ ID NO. 78753 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 8363, or a homolog or fragment thereof, or a homolog thereof being depicted in Table I, application no. 4, column 8, in the same line as SEQ ID NO. 8363, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 8363 is depicted, plastidic is mentioned, or a mitochondrial transit peptide in case in Table I, application no. 4, in column 6 in the same line as SEQ ID NO. 8363 is depicted, mitochondrial is mentioned; which is operable linked, too.

In another embodiment thereof the expression cassette does not comprise an additional transit peptide in case in Table I, application no. 4, in column 6 in the same line as SEQ ID NO. 8363 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 79181, or a homolog or fragment thereof, or a homolog thereof being depicted in Table I, application no. 4, column 8, in the same line as SEQ ID NO. 79181, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 79181 is depicted, plastidic is mentioned, or a mitochondrial transit peptide in case in Table I, application no. 4, in column 6 in the same line as SEQ ID NO. 79181 is depicted, mitochondrial is mentioned; which is operable linked, too.

In another embodiment thereof the expression cassette does not comprise an additional transit peptide in case in Table I, application no. 4, in column 6 in the same line as SEQ ID NO. 79181 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38947, or a homolog or fragment thereof, or a homolog thereof being depicted in Table I, application no. 4, column 8, in the same line as SEQ ID NO. 38947, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 38947 is depicted, plastidic is mentioned, or a mitochondrial transit peptide in case in Table I, application no. 4, in column 6 in the same line as SEQ ID NO. 38947 is depicted, mitochondrial is mentioned; which is operable linked, too.

In another embodiment thereof the expression cassette does not comprise an additional transit peptide in case in Table I, application no. 4, in column 6 in the same line as SEQ ID NO. 38947 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39219, or a homolog or fragment thereof, or a homolog thereof being depicted in Table I, application no. 4, column 8, in the same line as SEQ ID NO. 39219, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 39219 is depicted, plastidic is mentioned, or a mitochondrial transit peptide in case in Table I, application no. 4, in column 6 in the same line as SEQ ID NO. 39219 is depicted, mitochondrial is mentioned; which is operable linked, too.

In another embodiment thereof the expression cassette does not comprise an additional transit peptide in case in Table I, application no. 4, in column 6 in the same line as SEQ ID NO. 39219 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 79342, or a homolog or fragment thereof, or a homolog thereof being depicted in Table I, application no. 4, column 8, in the same line as SEQ ID NO. 79342, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 79342 is depicted, plastidic is mentioned, or a mitochondrial transit peptide in case in Table I, application no. 4, in column 6 in the same line as SEQ ID NO. 79342 is depicted, mitochondrial is mentioned; which is operable linked, too.

In another embodiment thereof the expression cassette does not comprise an additional transit peptide in case in Table I, application no. 4, in column 6 in the same line as SEQ ID NO. 79342 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39237, or a homolog or fragment thereof, or a homolog thereof being depicted in Table I, application no. 4, column 8, in the same line as SEQ ID NO. 39237, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 39237 is depicted, plastidic is mentioned, or a mitochondrial transit peptide in case in Table I, application no. 4, in column 6 in the same line as SEQ ID NO. 39237 is depicted, mitochondrial is mentioned; which is operable linked, too.

In another embodiment thereof the expression cassette does not comprise an additional transit peptide in case in Table I, application no. 4, in column 6 in the same line as SEQ ID NO. 39237 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39255, or a homolog or fragment thereof, or a homolog thereof being depicted in Table I, application no. 4, column 8, in the same line as SEQ ID NO. 39255, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 39255 is depicted, plastidic is mentioned, or a mitochondrial transit peptide in case in Table I, application no. 4, in column 6 in the same line as SEQ ID NO. 39255 is depicted, mitochondrial is mentioned; which is operable linked, too.

In another embodiment thereof the expression cassette does not comprise an additional transit peptide in case in Table I, application no. 4, in column 6 in the same line as SEQ ID NO. 39255 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 9167, or a homolog or fragment thereof, or a homolog thereof being depicted in Table I, application no. 4, column 8, in the same line as SEQ ID NO. 9167, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 9167 is depicted, plastidic is mentioned, or a mitochondrial transit peptide in case in Table I, application no. 4, in column 6 in the same line as SEQ ID NO. 9167 is depicted, mitochondrial is mentioned; which is operable linked, too.

In another embodiment thereof the expression cassette does not comprise an additional transit peptide in case in Table I, application no. 4, in column 6 in the same line as SEQ ID NO. 9167 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40665, or a homolog or fragment thereof, or a homolog thereof being depicted in Table I, application no. 4, column 8, in the same line as SEQ ID NO. 40665, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 40665 is depicted, plastidic is mentioned, or a mitochondrial transit peptide in case in Table I, application no. 4, in column 6 in the same line as SEQ ID NO. 40665 is depicted, mitochondrial is mentioned; which is operable linked, too.

In another embodiment thereof the expression cassette does not comprise an additional transit peptide in case in Table I, application no. 4, in column 6 in the same line as SEQ ID NO. 40665 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 80756, or a homolog or fragment thereof, or a homolog thereof being depicted in Table I, application no. 4, column 8, in the same line as SEQ ID NO. 80756, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 80756 is depicted, plastidic is mentioned, or a mitochondrial transit peptide in case in Table I, application no. 4, in column 6 in the same line as SEQ ID NO. 80756 is depicted, mitochondrial is mentioned; which is operable linked, too.

In another embodiment thereof the expression cassette does not comprise an additional transit peptide in case in Table I, application no. 4, in column 6 in the same line as SEQ ID NO. 80756 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40741, or a homolog or fragment thereof, or a homolog thereof being depicted in Table I, application no. 4, column 8, in the same line as SEQ ID NO. 40741, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 40741 is depicted, plastidic is mentioned, or a mitochondrial transit peptide in case in Table I, application no. 4, in column 6 in the same line as SEQ ID NO. 40741 is depicted, mitochondrial is mentioned; which is operable linked, too.

In another embodiment thereof the expression cassette does not comprise an additional transit peptide in case in Table I, application no. 4, in column 6 in the same line as SEQ ID NO. 40741 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 80906, or a homolog or fragment thereof, or a homolog thereof being depicted in Table I, application no. 4, column 8, in the same line as SEQ ID NO. 80906, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 80906 is depicted, plastidic is mentioned, or a mitochondrial transit peptide in case in Table I, application no. 4, in column 6 in the same line as SEQ ID NO. 80906 is depicted, mitochondrial is mentioned; which is operable linked, too.

In another embodiment thereof the expression cassette does not comprise an additional transit peptide in case in Table I, application no. 4, in column 6 in the same line as SEQ ID NO. 80906 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 81075, or a homolog or fragment thereof, or a homolog thereof being depicted in Table I, application no. 4, column 8, in the same line as SEQ ID NO. 81075, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 81075 is depicted, plastidic is mentioned, or a mitochondrial transit peptide in case in Table I, application no. 4, in column 6 in the same line as SEQ ID NO. 81075 is depicted, mitochondrial is mentioned; which is operable linked, too.

In another embodiment thereof the expression cassette does not comprise an additional transit peptide in case in Table I, application no. 4, in column 6 in the same line as SEQ ID NO. 81075 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40795, or a homolog or fragment thereof, or a homolog thereof being depicted in Table I, application no. 4, column 8, in the same line as SEQ ID NO. 40795, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 40795 is depicted, plastidic is mentioned, or a mitochondrial transit peptide in case in Table I, application no. 4, in column 6 in the same line as SEQ ID NO. 40795 is depicted, mitochondrial is mentioned; which is operable linked, too.

In another embodiment thereof the expression cassette does not comprise an additional transit peptide in case in Table I, application no. 4, in column 6 in the same line as SEQ ID NO. 40795 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40795, or a homolog or fragment thereof, or a homolog thereof being depicted in Table I, application no. 4, column 8, in the same line as SEQ ID NO. 40795, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 40795 is depicted, plastidic is mentioned, or a mitochondrial transit peptide in case in Table I, application no. 4, in column 6 in the same line as SEQ ID NO. 40795 is depicted, mitochondrial is mentioned; which is operable linked, too.

In another embodiment thereof the expression cassette does not comprise an additional transit peptide in case in Table I, application no. 4, in column 6 in the same line as SEQ ID NO. 40795 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 81988, or a homolog or fragment thereof, or a homolog thereof being depicted in Table I, application no. 4, column 8, in the same line as SEQ ID NO. 81988, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 81988 is depicted, plastidic is mentioned, or a mitochondrial transit peptide in case in Table I, application no. 4, in column 6 in the same line as SEQ ID NO. 81988 is depicted, mitochondrial is mentioned; which is operable linked, too.

In another embodiment thereof the expression cassette does not comprise an additional transit peptide in case in Table I, application no. 4, in column 6 in the same line as SEQ ID NO. 81988 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 41499, or a homolog or fragment thereof, or a homolog thereof being depicted in Table I, application no. 4, column 8, in the same line as SEQ ID NO. 41499, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 41499 is depicted, plastidic is mentioned, or a mitochondrial transit peptide in case in Table I, application no. 4, in column 6 in the same line as SEQ ID NO. 41499 is depicted, mitochondrial is mentioned; which is operable linked, too.

In another embodiment thereof the expression cassette does not comprise an additional transit peptide in case in Table I, application no. 4, in column 6 in the same line as SEQ ID NO. 41499 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 42046, or a homolog or fragment thereof, or a homolog thereof being depicted in Table I, application no. 4, column 8, in the same line as SEQ ID NO. 42046, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 42046 is depicted, plastidic is mentioned, or a mitochondrial transit peptide in case in Table I, application no. 4, in column 6 in the same line as SEQ ID NO. 42046 is depicted, mitochondrial is mentioned; which is operable linked, too.

In another embodiment thereof the expression cassette does not comprise an additional transit peptide in case in Table I, application no. 4, in column 6 in the same line as SEQ ID NO. 42046 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 82424, or a homolog or fragment thereof, or a homolog thereof being depicted in Table I, application no. 4, column 8, in the same line as SEQ ID NO. 82424, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 82424 is depicted, plastidic is mentioned, or a mitochondrial transit peptide in case in Table I, application no. 4, in column 6 in the same line as SEQ ID NO. 82424 is depicted, mitochondrial is mentioned; which is operable linked, too.

In another embodiment thereof the expression cassette does not comprise an additional transit peptide in case in Table I, application no. 4, in column 6 in the same line as SEQ ID NO. 82424 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 42502, or a homolog or fragment thereof, or a homolog thereof being depicted in Table I, application no. 4, column 8, in the same line as SEQ ID NO. 42502, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 42502 is depicted, plastidic is mentioned, or a mitochondrial transit peptide in case in Table I, application no. 4, in column 6 in the same line as SEQ ID NO. 42502 is depicted, mitochondrial is mentioned; which is operable linked, too.

In another embodiment thereof the expression cassette does not comprise an additional transit peptide in case in Table I, application no. 4, in column 6 in the same line as SEQ ID NO. 42502 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 94051, or a homolog or fragment thereof, or a homolog thereof being depicted in Table I, application no. 4, column 8, in the same line as SEQ ID NO. 94051, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 94051 is depicted, plastidic is mentioned, or a mitochondrial transit peptide in case in Table I, application no. 4, in column 6 in the same line as SEQ ID NO. 94051 is depicted, mitochondrial is mentioned; which is operable linked, too.

In another embodiment thereof the expression cassette does not comprise an additional transit peptide in case in Table I, application no. 4, in column 6 in the same line as SEQ ID NO. 94051 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 94380, or a homolog or fragment thereof, or a homolog thereof being depicted in Table I, application no. 4, column 8, in the same line as SEQ ID NO. 94380, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 94380 is depicted, plastidic is mentioned, or a mitochondrial transit peptide in case in Table I, application no. 4, in column 6 in the same line as SEQ ID NO. 94380 is depicted, mitochondrial is mentioned; which is operable linked, too.

In another embodiment thereof the expression cassette does not comprise an additional transit peptide in case in Table I, application no. 4, in column 6 in the same line as SEQ ID NO. 94380 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 10172, or a homolog or fragment thereof, or a homolog thereof being depicted in Table I, application no. 4, column 8, in the same line as SEQ ID NO. 10172, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 10172 is depicted, plastidic is mentioned, or a mitochondrial transit peptide in case in Table I, application no. 4, in column 6 in the same line as SEQ ID NO. 10172 is depicted, mitochondrial is mentioned; which is operable linked, too.

In another embodiment thereof the expression cassette does not comprise an additional transit peptide in case in Table I, application no. 4, in column 6 in the same line as SEQ ID NO. 10172 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 83429, or a homolog or fragment thereof, or a homolog thereof being depicted in Table I, application no. 4, column 8, in the same line as SEQ ID NO. 83429, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 83429 is depicted, plastidic is mentioned, or a mitochondrial transit peptide in case in Table I, application no. 4, in column 6 in the same line as SEQ ID NO. 83429 is depicted, mitochondrial is mentioned; which is operable linked, too.

In another embodiment thereof the expression cassette does not comprise an additional transit peptide in case in Table I, application no. 4, in column 6 in the same line as SEQ ID NO. 83429 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 43248, or a homolog or fragment thereof, or a homolog thereof being depicted in Table I, application no. 4, column 8, in the same line as SEQ ID NO. 43248, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 43248 is depicted, plastidic is mentioned, or a mitochondrial transit peptide in case in Table I, application no. 4, in column 6 in the same line as SEQ ID NO. 43248 is depicted, mitochondrial is mentioned; which is operable linked, too.

In another embodiment thereof the expression cassette does not comprise an additional transit peptide in case in Table I, application no. 4, in column 6 in the same line as SEQ ID NO. 43248 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 10252, or a homolog or fragment thereof, or a homolog thereof being depicted in Table I, application no. 4, column 8, in the same line as SEQ ID NO. 10252, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 10252 is depicted, plastidic is mentioned, or a mitochondrial transit peptide in case in Table I, application no. 4, in column 6 in the same line as SEQ ID NO. 10252 is depicted, mitochondrial is mentioned; which is operable linked, too.

In another embodiment thereof the expression cassette does not comprise an additional transit peptide in case in Table I, application no. 4, in column 6 in the same line as SEQ ID NO. 10252 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 94386, or a homolog or fragment thereof, or a homolog thereof being depicted in Table I, application no. 4, column 8, in the same line as SEQ ID NO. 94386, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 94386 is depicted, plastidic is mentioned, or a mitochondrial transit peptide in case in Table I, application no. 4, in column 6 in the same line as SEQ ID NO. 94386 is depicted, mitochondrial is mentioned; which is operable linked, too.

In another embodiment thereof the expression cassette does not comprise an additional transit peptide in case in Table I, application no. 4, in column 6 in the same line as SEQ ID NO. 94386 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 43800, or a homolog or fragment thereof, or a homolog thereof being depicted in Table I, application no. 4, column 8, in the same line as SEQ ID NO. 43800, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 43800 is depicted, plastidic is mentioned, or a mitochondrial transit peptide in case in Table I, application no. 4, in column 6 in the same line as SEQ ID NO. 43800 is depicted, mitochondrial is mentioned; which is operable linked, too.

In another embodiment thereof the expression cassette does not comprise an additional transit peptide in case in Table I, application no. 4, in column 6 in the same line as SEQ ID NO. 43800 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 43839, or a homolog or fragment thereof, or a homolog thereof being depicted in Table I, application no. 4, column 8, in the same line as SEQ ID NO. 43839, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 43839 is depicted, plastidic is mentioned, or a mitochondrial transit peptide in case in Table I, application no. 4, in column 6 in the same line as SEQ ID NO. 43839 is depicted, mitochondrial is mentioned; which is operable linked, too.

In another embodiment thereof the expression cassette does not comprise an additional transit peptide in case in Table I, application no. 4, in column 6 in the same line as SEQ ID NO. 43839 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 83821, or a homolog or fragment thereof, or a homolog thereof being depicted in Table I, application no. 4, column 8, in the same line as SEQ ID NO. 83821, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 83821 is depicted, plastidic is mentioned, or a mitochondrial transit peptide in case in Table I, application no. 4, in column 6 in the same line as SEQ ID NO. 83821 is depicted, mitochondrial is mentioned; which is operable linked, too.

In another embodiment thereof the expression cassette does not comprise an additional transit peptide in case in Table I, application no. 4, in column 6 in the same line as SEQ ID NO. 83821 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 44372, or a homolog or fragment thereof, or a homolog thereof being depicted in Table I, application no. 4, column 8, in the same line as SEQ ID NO. 44372, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 44372 is depicted, plastidic is mentioned, or a mitochondrial transit peptide in case in Table I, application no. 4, in column 6 in the same line as SEQ ID NO. 44372 is depicted, mitochondrial is mentioned; which is operable linked, too.

In another embodiment thereof the expression cassette does not comprise an additional transit peptide in case in Table I, application no. 4, in column 6 in the same line as SEQ ID NO. 44372 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 83835, or a homolog or fragment thereof, or a homolog thereof being depicted in Table I, application no. 4, column 8, in the same line as SEQ ID NO. 83835, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 83835 is depicted, plastidic is mentioned, or a mitochondrial transit peptide in case in Table I, application no. 4, in column 6 in the same line as SEQ ID NO. 83835 is depicted, mitochondrial is mentioned; which is operable linked, too.

In another embodiment thereof the expression cassette does not comprise an additional transit peptide in case in Table I, application no. 4, in column 6 in the same line as SEQ ID NO. 83835 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 10740, or a homolog or fragment thereof, or a homolog thereof being depicted in Table I, application no. 4, column 8, in the same line as SEQ ID NO. 10740, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 10740 is depicted, plastidic is mentioned, or a mitochondrial transit peptide in case in Table I, application no. 4, in column 6 in the same line as SEQ ID NO. 10740 is depicted, mitochondrial is mentioned; which is operable linked, too.

In another embodiment thereof the expression cassette does not comprise an additional transit peptide in case in Table I, application no. 4, in column 6 in the same line as SEQ ID NO. 10740 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 94542, or a homolog or fragment thereof, or a homolog thereof being depicted in Table I, application no. 4, column 8, in the same line as SEQ ID NO. 94542, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 94542 is depicted, plastidic is mentioned, or a mitochondrial transit peptide in case in Table I, application no. 4, in column 6 in the same line as SEQ ID NO. 94542 is depicted, mitochondrial is mentioned; which is operable linked, too.

In another embodiment thereof the expression cassette does not comprise an additional transit peptide in case in Table I, application no. 4, in column 6 in the same line as SEQ ID NO. 94542 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 94735, or a homolog or fragment thereof, or a homolog thereof being depicted in Table I, application no. 4, column 8, in the same line as SEQ ID NO. 94735, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 94735 is depicted, plastidic is mentioned, or a mitochondrial transit peptide in case in Table I, application no. 4, in column 6 in the same line as SEQ ID NO. 94735 is depicted, mitochondrial is mentioned; which is operable linked, too.

In another embodiment thereof the expression cassette does not comprise an additional transit peptide in case in Table I, application no. 4, in column 6 in the same line as SEQ ID NO. 94735 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 45321, or a homolog or fragment thereof, or a homolog thereof being depicted in Table I, application no. 4, column 8, in the same line as SEQ ID NO. 45321, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 45321 is depicted, plastidic is mentioned, or a mitochondrial transit peptide in case in Table I, application no. 4, in column 6 in the same line as SEQ ID NO. 45321 is depicted, mitochondrial is mentioned; which is operable linked, too.

In another embodiment thereof the expression cassette does not comprise an additional transit peptide in case in Table I, application no. 4, in column 6 in the same line as SEQ ID NO. 45321 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 10811, or a homolog or fragment thereof, or a homolog thereof being depicted in Table I, application no. 4, column 8, in the same line as SEQ ID NO. 10811, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 10811 is depicted, plastidic is mentioned, or a mitochondrial transit peptide in case in Table I, application no. 4, in column 6 in the same line as SEQ ID NO. 10811 is depicted, mitochondrial is mentioned; which is operable linked, too.

In another embodiment thereof the expression cassette does not comprise an additional transit peptide in case in Table I, application no. 4, in column 6 in the same line as SEQ ID NO. 10811 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 84198, or a homolog or fragment thereof, or a homolog thereof being depicted in Table I, application no. 4, column 8, in the same line as SEQ ID NO. 84198, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 84198 is depicted, plastidic is mentioned, or a mitochondrial transit peptide in case in Table I, application no. 4, in column 6 in the same line as SEQ ID NO. 84198 is depicted, mitochondrial is mentioned; which is operable linked, too.

In another embodiment thereof the expression cassette does not comprise an additional transit peptide in case in Table I, application no. 4, in column 6 in the same line as SEQ ID NO. 84198 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 46850, or a homolog or fragment thereof, or a homolog thereof being depicted in Table I, application no. 4, column 8, in the same line as SEQ ID NO. 46850, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 46850 is depicted, plastidic is mentioned, or a mitochondrial transit peptide in case in Table I, application no. 4, in column 6 in the same line as SEQ ID NO. 46850 is depicted, mitochondrial is mentioned; which is operable linked, too.

In another embodiment thereof the expression cassette does not comprise an additional transit peptide in case in Table I, application no. 4, in column 6 in the same line as SEQ ID NO. 46850 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 48138, or a homolog or fragment thereof, or a homolog thereof being depicted in Table I, application no. 4, column 8, in the same line as SEQ ID NO. 48138, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 48138 is depicted, plastidic is mentioned, or a mitochondrial transit peptide in case in Table I, application no. 4, in column 6 in the same line as SEQ ID NO. 48138 is depicted, mitochondrial is mentioned; which is operable linked, too.

In another embodiment thereof the expression cassette does not comprise an additional transit peptide in case in Table I, application no. 4, in column 6 in the same line as SEQ ID NO. 48138 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 49828, or a homolog or fragment thereof, or a homolog thereof being depicted in Table I, application no. 4, column 8, in the same line as SEQ ID NO. 49828, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 49828 is depicted, plastidic is mentioned, or a mitochondrial transit peptide in case in Table I, application no. 4, in column 6 in the same line as SEQ ID NO. 49828 is depicted, mitochondrial is mentioned; which is operable linked, too.

In another embodiment thereof the expression cassette does not comprise an additional transit peptide in case in Table I, application no. 4, in column 6 in the same line as SEQ ID NO. 49828 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 94741, or a homolog or fragment thereof, or a homolog thereof being depicted in Table I, application no. 4, column 8, in the same line as SEQ ID NO. 94741, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 94741 is depicted, plastidic is mentioned, or a mitochondrial transit peptide in case in Table I, application no. 4, in column 6 in the same line as SEQ ID NO. 94741 is depicted, mitochondrial is mentioned; which is operable linked, too.

In another embodiment thereof the expression cassette does not comprise an additional transit peptide in case in Table I, application no. 4, in column 6 in the same line as SEQ ID NO. 94741 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 84869, or a homolog or fragment thereof, or a homolog thereof being depicted in Table I, application no. 4, column 8, in the same line as SEQ ID NO. 84869, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 84869 is depicted, plastidic is mentioned, or a mitochondrial transit peptide in case in Table I, application no. 4, in column 6 in the same line as SEQ ID NO. 84869 is depicted, mitochondrial is mentioned; which is operable linked, too.

In another embodiment thereof the expression cassette does not comprise an additional transit peptide in case in Table I, application no. 4, in column 6 in the same line as SEQ ID NO. 84869 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 94942, or a homolog or fragment thereof, or a homolog thereof being depicted in Table I, application no. 4, column 8, in the same line as SEQ ID NO. 94942, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 94942 is depicted, plastidic is mentioned, or a mitochondrial transit peptide in case in Table I, application no. 4, in column 6 in the same line as SEQ ID NO. 94942 is depicted, mitochondrial is mentioned; which is operable linked, too.

In another embodiment thereof the expression cassette does not comprise an additional transit peptide in case in Table I, application no. 4, in column 6 in the same line as SEQ ID NO. 94942 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 51198, or a homolog or fragment thereof, or a homolog thereof being depicted in Table I, application no. 4, column 8, in the same line as SEQ ID NO. 51198, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 51198 is depicted, plastidic is mentioned, or a mitochondrial transit peptide in case in Table I, application no. 4, in column 6 in the same line as SEQ ID NO. 51198 is depicted, mitochondrial is mentioned; which is operable linked, too.

In another embodiment thereof the expression cassette does not comprise an additional transit peptide in case in Table I, application no. 4, in column 6 in the same line as SEQ ID NO. 51198 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 51268, or a homolog or fragment thereof, or a homolog thereof being depicted in Table I, application no. 4, column 8, in the same line as SEQ ID NO. 51268, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 51268 is depicted, plastidic is mentioned, or a mitochondrial transit peptide in case in Table I, application no. 4, in column 6 in the same line as SEQ ID NO. 51268 is depicted, mitochondrial is mentioned; which is operable linked, too.

In another embodiment thereof the expression cassette does not comprise an additional transit peptide in case in Table I, application no. 4, in column 6 in the same line as SEQ ID NO. 51268 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 51632, or a homolog or fragment thereof, or a homolog thereof being depicted in Table I, application no. 4, column 8, in the same line as SEQ ID NO. 51632, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 51632 is depicted, plastidic is mentioned, or a mitochondrial transit peptide in case in Table I, application no. 4, in column 6 in the same line as SEQ ID NO. 51632 is depicted, mitochondrial is mentioned; which is operable linked, too.

In another embodiment thereof the expression cassette does not comprise an additional transit peptide in case in Table I, application no. 4, in column 6 in the same line as SEQ ID NO. 51632 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 52364, or a homolog or fragment thereof, or a homolog thereof being depicted in Table I, application no. 4, column 8, in the same line as SEQ ID NO. 52364, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 52364 is depicted, plastidic is mentioned, or a mitochondrial transit peptide in case in Table I, application no. 4, in column 6 in the same line as SEQ ID NO. 52364 is depicted, mitochondrial is mentioned; which is operable linked, too.

In another embodiment thereof the expression cassette does not comprise an additional transit peptide in case in Table I, application no. 4, in column 6 in the same line as SEQ ID NO. 52364 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 53456, or a homolog or fragment thereof, or a homolog thereof being depicted in Table I, application no. 4, column 8, in the same line as SEQ ID NO. 53456, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 53456 is depicted, plastidic is mentioned, or a mitochondrial transit peptide in case in Table I, application no. 4, in column 6 in the same line as SEQ ID NO. 53456 is depicted, mitochondrial is mentioned; which is operable linked, too.

In another embodiment thereof the expression cassette does not comprise an additional transit peptide in case in Table I, application no. 4, in column 6 in the same line as SEQ ID NO. 53456 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 54337, or a homolog or fragment thereof, or a homolog thereof being depicted in Table I, application no. 4, column 8, in the same line as SEQ ID NO. 54337, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 54337 is depicted, plastidic is mentioned, or a mitochondrial transit peptide in case in Table I, application no. 4, in column 6 in the same line as SEQ ID NO. 54337 is depicted, mitochondrial is mentioned; which is operable linked, too.

In another embodiment thereof the expression cassette does not comprise an additional transit peptide in case in Table I, application no. 4, in column 6 in the same line as SEQ ID NO. 54337 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 95047, or a homolog or fragment thereof, or a homolog thereof being depicted in Table I, application no. 4, column 8, in the same line as SEQ ID NO. 95047, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 95047 is depicted, plastidic is mentioned, or a mitochondrial transit peptide in case in Table I, application no. 4, in column 6 in the same line as SEQ ID NO. 95047 is depicted, mitochondrial is mentioned; which is operable linked, too.

In another embodiment thereof the expression cassette does not comprise an additional transit peptide in case in Table I, application no. 4, in column 6 in the same line as SEQ ID NO. 95047 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 11423, or a homolog or fragment thereof, or a homolog thereof being depicted in Table I, application no. 4, column 8, in the same line as SEQ ID NO. 11423, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 11423 is depicted, plastidic is mentioned, or a mitochondrial transit peptide in case in Table I, application no. 4, in column 6 in the same line as SEQ ID NO. 11423 is depicted, mitochondrial is mentioned; which is operable linked, too.

In another embodiment thereof the expression cassette does not comprise an additional transit peptide in case in Table I, application no. 4, in column 6 in the same line as SEQ ID NO. 11423 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 85743, or a homolog or fragment thereof, or a homolog thereof being depicted in Table I, application no. 4, column 8, in the same line as SEQ ID NO. 85743, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 85743 is depicted, plastidic is mentioned, or a mitochondrial transit peptide in case in Table I, application no. 4, in column 6 in the same line as SEQ ID NO. 85743 is depicted, mitochondrial is mentioned; which is operable linked, too.

In another embodiment thereof the expression cassette does not comprise an additional transit peptide in case in Table I, application no. 4, in column 6 in the same line as SEQ ID NO. 85743 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 11471, or a homolog or fragment thereof, or a homolog thereof being depicted in Table I, application no. 4, column 8, in the same line as SEQ ID NO. 11471, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 11471 is depicted, plastidic is mentioned, or a mitochondrial transit peptide in case in Table I, application no. 4, in column 6 in the same line as SEQ ID NO. 11471 is depicted, mitochondrial is mentioned; which is operable linked, too.

In another embodiment thereof the expression cassette does not comprise an additional transit peptide in case in Table I, application no. 4, in column 6 in the same line as SEQ ID NO. 11471 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 95527, or a homolog or fragment thereof, or a homolog thereof being depicted in Table I, application no. 4, column 8, in the same line as SEQ ID NO. 95527, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 95527 is depicted, plastidic is mentioned, or a mitochondrial transit peptide in case in Table I, application no. 4, in column 6 in the same line as SEQ ID NO. 95527 is depicted, mitochondrial is mentioned; which is operable linked, too.

In another embodiment thereof the expression cassette does not comprise an additional transit peptide in case in Table I, application no. 4, in column 6 in the same line as SEQ ID NO. 95527 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 95744, or a homolog or fragment thereof, or a homolog thereof being depicted in Table I, application no. 4, column 8, in the same line as SEQ ID NO. 95744, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 95744 is depicted, plastidic is mentioned, or a mitochondrial transit peptide in case in Table I, application no. 4, in column 6 in the same line as SEQ ID NO. 95744 is depicted, mitochondrial is mentioned; which is operable linked, too.

In another embodiment thereof the expression cassette does not comprise an additional transit peptide in case in Table I, application no. 4, in column 6 in the same line as SEQ ID NO. 95744 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 56153, or a homolog or fragment thereof, or a homolog thereof being depicted in Table I, application no. 4, column 8, in the same line as SEQ ID NO. 56153, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 56153 is depicted, plastidic is mentioned, or a mitochondrial transit peptide in case in Table I, application no. 4, in column 6 in the same line as SEQ ID NO. 56153 is depicted, mitochondrial is mentioned; which is operable linked, too.

In another embodiment thereof the expression cassette does not comprise an additional transit peptide in case in Table I, application no. 4, in column 6 in the same line as SEQ ID NO. 56153 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 95857, or a homolog or fragment thereof, or a homolog thereof being depicted in Table I, application no. 4, column 8, in the same line as SEQ ID NO. 95857, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 95857 is depicted, plastidic is mentioned, or a mitochondrial transit peptide in case in Table I, application no. 4, in column 6 in the same line as SEQ ID NO. 95857 is depicted, mitochondrial is mentioned; which is operable linked, too.

In another embodiment thereof the expression cassette does not comprise an additional transit peptide in case in Table I, application no. 4, in column 6 in the same line as SEQ ID NO. 95857 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 57235, or a homolog or fragment thereof, or a homolog thereof being depicted in Table I, application no. 4, column 8, in the same line as SEQ ID NO. 57235, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 57235 is depicted, plastidic is mentioned, or a mitochondrial transit peptide in case in Table I, application no. 4, in column 6 in the same line as SEQ ID NO. 57235 is depicted, mitochondrial is mentioned; which is operable linked, too.

In another embodiment thereof the expression cassette does not comprise an additional transit peptide in case in Table I, application no. 4, in column 6 in the same line as SEQ ID NO. 57235 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 87610, or a homolog or fragment thereof, or a homolog thereof being depicted in Table I, application no. 4, column 8, in the same line as SEQ ID NO. 87610, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 87610 is depicted, plastidic is mentioned, or a mitochondrial transit peptide in case in Table I, application no. 4, in column 6 in the same line as SEQ ID NO. 87610 is depicted, mitochondrial is mentioned; which is operable linked, too.

In another embodiment thereof the expression cassette does not comprise an additional transit peptide in case in Table I, application no. 4, in column 6 in the same line as SEQ ID NO. 87610 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 87853, or a homolog or fragment thereof, or a homolog thereof being depicted in Table I, application no. 4, column 8, in the same line as SEQ ID NO. 87853, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 87853 is depicted, plastidic is mentioned, or a mitochondrial transit peptide in case in Table I, application no. 4, in column 6 in the same line as SEQ ID NO. 87853 is depicted, mitochondrial is mentioned; which is operable linked, too.

In another embodiment thereof the expression cassette does not comprise an additional transit peptide in case in Table I, application no. 4, in column 6 in the same line as SEQ ID NO. 87853 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 58731, or a homolog or fragment thereof, or a homolog thereof being depicted in Table I, application no. 4, column 8, in the same line as SEQ ID NO. 58731, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 58731 is depicted, plastidic is mentioned, or a mitochondrial transit peptide in case in Table I, application no. 4, in column 6 in the same line as SEQ ID NO. 58731 is depicted, mitochondrial is mentioned; which is operable linked, too.

In another embodiment thereof the expression cassette does not comprise an additional transit peptide in case in Table I, application no. 4, in column 6 in the same line as SEQ ID NO. 58731 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 60301, or a homolog or fragment thereof, or a homolog thereof being depicted in Table I, application no. 4, column 8, in the same line as SEQ ID NO. 60301, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 60301 is depicted, plastidic is mentioned, or a mitochondrial transit peptide in case in Table I, application no. 4, in column 6 in the same line as SEQ ID NO. 60301 is depicted, mitochondrial is mentioned; which is operable linked, too.

In another embodiment thereof the expression cassette does not comprise an additional transit peptide in case in Table I, application no. 4, in column 6 in the same line as SEQ ID NO. 60301 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 60859, or a homolog or fragment thereof, or a homolog thereof being depicted in Table I, application no. 4, column 8, in the same line as SEQ ID NO. 60859, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 60859 is depicted, plastidic is mentioned, or a mitochondrial transit peptide in case in Table I, application no. 4, in column 6 in the same line as SEQ ID NO. 60859 is depicted, mitochondrial is mentioned; which is operable linked, too.

In another embodiment thereof the expression cassette does not comprise an additional transit peptide in case in Table I, application no. 4, in column 6 in the same line as SEQ ID NO. 60859 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 88875, or a homolog or fragment thereof, or a homolog thereof being depicted in Table I, application no. 4, column 8, in the same line as SEQ ID NO. 88875, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 88875 is depicted, plastidic is mentioned, or a mitochondrial transit peptide in case in Table I, application no. 4, in column 6 in the same line as SEQ ID NO. 88875 is depicted, mitochondrial is mentioned; which is operable linked, too.

In another embodiment thereof the expression cassette does not comprise an additional transit peptide in case in Table I, application no. 4, in column 6 in the same line as SEQ ID NO. 88875 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 61553, or a homolog or fragment thereof, or a homolog thereof being depicted in Table I, application no. 4, column 8, in the same line as SEQ ID NO. 61553, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 61553 is depicted, plastidic is mentioned, or a mitochondrial transit peptide in case in Table I, application no. 4, in column 6 in the same line as SEQ ID NO. 61553 is depicted, mitochondrial is mentioned; which is operable linked, too.

In another embodiment thereof the expression cassette does not comprise an additional transit peptide in case in Table I, application no. 4, in column 6 in the same line as SEQ ID NO. 61553 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12974, or a homolog or fragment thereof, or a homolog thereof being depicted in Table I, application no. 4, column 8, in the same line as SEQ ID NO. 12974, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 12974 is depicted, plastidic is mentioned, or a mitochondrial transit peptide in case in Table I, application no. 4, in column 6 in the same line as SEQ ID NO. 12974 is depicted, mitochondrial is mentioned; which is operable linked, too.

In another embodiment thereof the expression cassette does not comprise an additional transit peptide in case in Table I, application no. 4, in column 6 in the same line as SEQ ID NO. 12974 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 62244, or a homolog or fragment thereof, or a homolog thereof being depicted in Table I, application no. 4, column 8, in the same line as SEQ ID NO. 62244, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 62244 is depicted, plastidic is mentioned, or a mitochondrial transit peptide in case in Table I, application no. 4, in column 6 in the same line as SEQ ID NO. 62244 is depicted, mitochondrial is mentioned; which is operable linked, too.

In another embodiment thereof the expression cassette does not comprise an additional transit peptide in case in Table I, application no. 4, in column 6 in the same line as SEQ ID NO. 62244 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 96022, or a homolog or fragment thereof, or a homolog thereof being depicted in Table I, application no. 4, column 8, in the same line as SEQ ID NO. 96022, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 96022 is depicted, plastidic is mentioned, or a mitochondrial transit peptide in case in Table I, application no. 4, in column 6 in the same line as SEQ ID NO. 96022 is depicted, mitochondrial is mentioned; which is operable linked, too.

In another embodiment thereof the expression cassette does not comprise an additional transit peptide in case in Table I, application no. 4, in column 6 in the same line as SEQ ID NO. 96022 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 62524, or a homolog or fragment thereof, or a homolog thereof being depicted in Table I, application no. 4, column 8, in the same line as SEQ ID NO. 62524, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 62524 is depicted, plastidic is mentioned, or a mitochondrial transit peptide in case in Table I, application no. 4, in column 6 in the same line as SEQ ID NO. 62524 is depicted, mitochondrial is mentioned; which is operable linked, too.

In another embodiment thereof the expression cassette does not comprise an additional transit peptide in case in Table I, application no. 4, in column 6 in the same line as SEQ ID NO. 62524 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 62717, or a homolog or fragment thereof, or a homolog thereof being depicted in Table I, application no. 4, column 8, in the same line as SEQ ID NO. 62717, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 62717 is depicted, plastidic is mentioned, or a mitochondrial transit peptide in case in Table I, application no. 4, in column 6 in the same line as SEQ ID NO. 62717 is depicted, mitochondrial is mentioned; which is operable linked, too.

In another embodiment thereof the expression cassette does not comprise an additional transit peptide in case in Table I, application no. 4, in column 6 in the same line as SEQ ID NO. 62717 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 96779, or a homolog or fragment thereof, or a homolog thereof being depicted in Table I, application no. 4, column 8, in the same line as SEQ ID NO. 96779, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 96779 is depicted, plastidic is mentioned, or a mitochondrial transit peptide in case in Table I, application no. 4, in column 6 in the same line as SEQ ID NO. 96779 is depicted, mitochondrial is mentioned; which is operable linked, too.

In another embodiment thereof the expression cassette does not comprise an additional transit peptide in case in Table I, application no. 4, in column 6 in the same line as SEQ ID NO. 96779 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 96980, or a homolog or fragment thereof, or a homolog thereof being depicted in Table I, application no. 4, column 8, in the same line as SEQ ID NO. 96980, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 96980 is depicted, plastidic is mentioned, or a mitochondrial transit peptide in case in Table I, application no. 4, in column 6 in the same line as SEQ ID NO. 96980 is depicted, mitochondrial is mentioned; which is operable linked, too.

In another embodiment thereof the expression cassette does not comprise an additional transit peptide in case in Table I, application no. 4, in column 6 in the same line as SEQ ID NO. 96980 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 63334, or a homolog or fragment thereof, or a homolog thereof being depicted in Table I, application no. 4, column 8, in the same line as SEQ ID NO. 63334, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 63334 is depicted, plastidic is mentioned, or a mitochondrial transit peptide in case in Table I, application no. 4, in column 6 in the same line as SEQ ID NO. 63334 is depicted, mitochondrial is mentioned; which is operable linked, too.

In another embodiment thereof the expression cassette does not comprise an additional transit peptide in case in Table I, application no. 4, in column 6 in the same line as SEQ ID NO. 63334 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 90103, or a homolog or fragment thereof, or a homolog thereof being depicted in Table I, application no. 4, column 8, in the same line as SEQ ID NO. 90103, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 90103 is depicted, plastidic is mentioned, or a mitochondrial transit peptide in case in Table I, application no. 4, in column 6 in the same line as SEQ ID NO. 90103 is depicted, mitochondrial is mentioned; which is operable linked, too.

In another embodiment thereof the expression cassette does not comprise an additional transit peptide in case in Table I, application no. 4, in column 6 in the same line as SEQ ID NO. 90103 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 96986, or a homolog or fragment thereof, or a homolog thereof being depicted in Table I, application no. 4, column 8, in the same line as SEQ ID NO. 96986, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 96986 is depicted, plastidic is mentioned, or a mitochondrial transit peptide in case in Table I, application no. 4, in column 6 in the same line as SEQ ID NO. 96986 is depicted, mitochondrial is mentioned; which is operable linked, too.

In another embodiment thereof the expression cassette does not comprise an additional transit peptide in case in Table I, application no. 4, in column 6 in the same line as SEQ ID NO. 96986 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 97294, or a homolog or fragment thereof, or a homolog thereof being depicted in Table I, application no. 4, column 8, in the same line as SEQ ID NO. 97294, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 97294 is depicted, plastidic is mentioned, or a mitochondrial transit peptide in case in Table I, application no. 4, in column 6 in the same line as SEQ ID NO. 97294 is depicted, mitochondrial is mentioned; which is operable linked, too.

In another embodiment thereof the expression cassette does not comprise an additional transit peptide in case in Table I, application no. 4, in column 6 in the same line as SEQ ID NO. 97294 is depicted, non-targeted is mentioned.

In an embodiment the present invention relates to an expresssion cassette comprising a) a promoter, preferably a promoter selected from the group consisting of Big355, PCUbi, Super and USP, in particular p-PcUBI; and b) the nucleic acid SEQ ID NO. 97298, or a homolog or fragment thereof, or a homolog thereof being depicted in Table I, application no. 4, column 8, in the same line as SEQ ID NO. 97298, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 97298 is depicted, plastidic is mentioned, or a mitochondrial transit peptide in case in Table I, application no. 4, in column 6 in the same line as SEQ ID NO. 97298 is depicted, mitochondrial is mentioned; which is operable linked, too.

In another embodiment thereof the expression cassette does not comprise an additional transit peptide in case in Table I, application no. 4, in column 6 in the same line as SEQ ID NO. 97298 is depicted, non-targeted is mentioned.

In an embodiment the present invention relates to an expresssion cassette comprising a) a promoter, preferably a promoter selected from the group consisting of Big355, PCUbi, Super and USP, in particular p-Super; and b) the nucleic acid SEQ ID NO. 64148, or a homolog or fragment thereof, or a homolog thereof being depicted in Table I, application no. 4, column 8, in the same line as SEQ ID NO. 64148, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 64148 is depicted, plastidic is mentioned, or a mitochondrial transit peptide in case in Table I, application no. 4, in column 6 in the same line as SEQ ID NO. 64148 is depicted, mitochondrial is mentioned; which is operable linked, too.

In another embodiment thereof the expression cassette does not comprise an additional transit peptide in case in Table I, application no. 4, in column 6 in the same line as SEQ ID NO. 64148 is depicted, non-targeted is mentioned.

In an embodiment the present invention relates to an expresssion cassette comprising a) a promoter, preferably a promoter selected from the group consisting of Big355, PCUbi, Super and USP, in particular Big355; and b) the nucleic acid SEQ ID NO. 64177, or a homolog or fragment thereof, or a homolog thereof being depicted in Table I, application no. 4, column 8, in the same line as SEQ ID NO. 64177, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 64177 is depicted, plastidic is mentioned, or a mitochondrial transit peptide in case in Table I, application no. 4, in column 6 in the same line as SEQ ID NO. 64177 is depicted, mitochondrial is mentioned; which is operable linked, too.

In another embodiment thereof the expression cassette does not comprise an additional transit peptide in case in Table I, application no. 4, in column 6 in the same line as SEQ ID NO. 64177 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 97620, or a homolog or fragment thereof, or a homolog thereof being depicted in Table I, application no. 4, column 8, in the same line as SEQ ID NO. 97620, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 97620 is depicted, plastidic is mentioned, or a mitochondrial transit peptide in case in Table I, application no. 4, in column 6 in the same line as SEQ ID NO. 97620 is depicted, mitochondrial is mentioned; which is operable linked, too.

In another embodiment thereof the expression cassette does not comprise an additional transit peptide in case in Table I, application no. 4, in column 6 in the same line as SEQ ID NO. 97620 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 14769, or a homolog or fragment thereof, or a homolog thereof being depicted in Table I, application no. 4, column 8, in the same line as SEQ ID NO. 14769, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 14769 is depicted, plastidic is mentioned, or a mitochondrial transit peptide in case in Table I, application no. 4, in column 6 in the same line as SEQ ID NO. 14769 is depicted, mitochondrial is mentioned; which is operable linked, too.

In another embodiment thereof the expression cassette does not comprise an additional transit peptide in case in Table I, application no. 4, in column 6 in the same line as SEQ ID NO. 14769 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 64563, or a homolog or fragment thereof, or a homolog thereof being depicted in Table I, application no. 4, column 8, in the same line as SEQ ID NO. 64563, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 64563 is depicted, plastidic is mentioned, or a mitochondrial transit peptide in case in Table I, application no. 4, in column 6 in the same line as SEQ ID NO. 64563 is depicted, mitochondrial is mentioned; which is operable linked, too.

In another embodiment thereof the expression cassette does not comprise an additional transit peptide in case in Table I, application no. 4, in column 6 in the same line as SEQ ID NO. 64563 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 90306, or a homolog or fragment thereof, or a homolog thereof being depicted in Table I, application no. 4, column 8, in the same line as SEQ ID NO. 90306, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 90306 is depicted, plastidic is mentioned, or a mitochondrial transit peptide in case in Table I, application no. 4, in column 6 in the same line as SEQ ID NO. 90306 is depicted, mitochondrial is mentioned; which is operable linked, too.

In another embodiment thereof the expression cassette does not comprise an additional transit peptide in case in Table I, application no. 4, in column 6 in the same line as SEQ ID NO. 90306 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 64964, or a homolog or fragment thereof, or a homolog thereof being depicted in Table I, application no. 4, column 8, in the same line as SEQ ID NO. 64964, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 64964 is depicted, plastidic is mentioned, or a mitochondrial transit peptide in case in Table I, application no. 4, in column 6 in the same line as SEQ ID NO. 64964 is depicted, mitochondrial is mentioned; which is operable linked, too.

In another embodiment thereof the expression cassette does not comprise an additional transit peptide in case in Table I, application no. 4, in column 6 in the same line as SEQ ID NO. 64964 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 97631, or a homolog or fragment thereof, or a homolog thereof being depicted in Table I, application no. 4, column 8, in the same line as SEQ ID NO. 97631, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 97631 is depicted, plastidic is mentioned, or a mitochondrial transit peptide in case in Table I, application no. 4, in column 6 in the same line as SEQ ID NO. 97631 is depicted, mitochondrial is mentioned; which is operable linked, too.

In another embodiment thereof the expression cassette does not comprise an additional transit peptide in case in Table I, application no. 4, in column 6 in the same line as SEQ ID NO. 97631 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 97635, or a homolog or fragment thereof, or a homolog thereof being depicted in Table I, application no. 4, column 8, in the same line as SEQ ID NO. 97635, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 97635 is depicted, plastidic is mentioned, or a mitochondrial transit peptide in case in Table I, application no. 4, in column 6 in the same line as SEQ ID NO. 97635 is depicted, mitochondrial is mentioned; which is operable linked, too.

In another embodiment thereof the expression cassette does not comprise an additional transit peptide in case in Table I, application no. 4, in column 6 in the same line as SEQ ID NO. 97635 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 14885, or a homolog or fragment thereof, or a homolog thereof being depicted in Table I, application no. 4, column 8, in the same line as SEQ ID NO. 14885, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 14885 is depicted, plastidic is mentioned, or a mitochondrial transit peptide in case in Table I, application no. 4, in column 6 in the same line as SEQ ID NO. 14885 is depicted, mitochondrial is mentioned; which is operable linked, too.

In another embodiment thereof the expression cassette does not comprise an additional transit peptide in case in Table I, application no. 4, in column 6 in the same line as SEQ ID NO. 14885 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 90409, or a homolog or fragment thereof, or a homolog thereof being depicted in Table I, application no. 4, column 8, in the same line as SEQ ID NO. 90409, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 90409 is depicted, plastidic is mentioned, or a mitochondrial transit peptide in case in Table I, application no. 4, in column 6 in the same line as SEQ ID NO. 90409 is depicted, mitochondrial is mentioned; which is operable linked, too.

In another embodiment thereof the expression cassette does not comprise an additional transit peptide in case in Table I, application no. 4, in column 6 in the same line as SEQ ID NO. 90409 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 90602, or a homolog or fragment thereof, or a homolog thereof being depicted in Table I, application no. 4, column 8, in the same line as SEQ ID NO. 90602, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 90602 is depicted, plastidic is mentioned, or a mitochondrial transit peptide in case in Table I, application no. 4, in column 6 in the same line as SEQ ID NO. 90602 is depicted, mitochondrial is mentioned; which is operable linked, too.

In another embodiment thereof the expression cassette does not comprise an additional transit peptide in case in Table I, application no. 4, in column 6 in the same line as SEQ ID NO. 90602 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 98075, or a homolog or fragment thereof, or a homolog thereof being depicted in Table I, application no. 4, column 8, in the same line as SEQ ID NO. 98075, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 98075 is depicted, plastidic is mentioned, or a mitochondrial transit peptide in case in Table I, application no. 4, in column 6 in the same line as SEQ ID NO. 98075 is depicted, mitochondrial is mentioned; which is operable linked, too.

In another embodiment thereof the expression cassette does not comprise an additional transit peptide in case in Table I, application no. 4, in column 6 in the same line as SEQ ID NO. 98075 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 90615, or a homolog or fragment thereof, or a homolog thereof being depicted in Table I, application no. 4, column 8, in the same line as SEQ ID NO. 90615, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 90615 is depicted, plastidic is mentioned, or a mitochondrial transit peptide in case in Table I, application no. 4, in column 6 in the same line as SEQ ID NO. 90615 is depicted, mitochondrial is mentioned; which is operable linked, too.

In another embodiment thereof the expression cassette does not comprise an additional transit peptide in case in Table I, application no. 4, in column 6 in the same line as SEQ ID NO. 90615 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 98243, or a homolog or fragment thereof, or a homolog thereof being depicted in Table I, application no. 4, column 8, in the same line as SEQ ID NO. 98243, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 98243 is depicted, plastidic is mentioned, or a mitochondrial transit peptide in case in Table I, application no. 4, in column 6 in the same line as SEQ ID NO. 98243 is depicted, mitochondrial is mentioned; which is operable linked, too.

In another embodiment thereof the expression cassette does not comprise an additional transit peptide in case in Table I, application no. 4, in column 6 in the same line as SEQ ID NO. 98243 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 91785, or a homolog or fragment thereof, or a homolog thereof being depicted in Table I, application no. 4, column 8, in the same line as SEQ ID NO. 91785, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 91785 is depicted, plastidic is mentioned, or a mitochondrial transit peptide in case in Table I, application no. 4, in column 6 in the same line as SEQ ID NO. 91785 is depicted, mitochondrial is mentioned; which is operable linked, too.

In another embodiment thereof the expression cassette does not comprise an additional transit peptide in case in Table I, application no. 4, in column 6 in the same line as SEQ ID NO. 91785 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 67684, or a homolog or fragment thereof, or a homolog thereof being depicted in Table I, application no. 4, column 8, in the same line as SEQ ID NO. 67684, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 67684 is depicted, plastidic is mentioned, or a mitochondrial transit peptide in case in Table I, application no. 4, in column 6 in the same line as SEQ ID NO. 67684 is depicted, mitochondrial is mentioned; which is operable linked, too.

In another embodiment thereof the expression cassette does not comprise an additional transit peptide in case in Table I, application no. 4, in column 6 in the same line as SEQ ID NO. 67684 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 92540, or a homolog or fragment thereof, or a homolog thereof being depicted in Table I, application no. 4, column 8, in the same line as SEQ ID NO. 92540, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 92540 is depicted, plastidic is mentioned, or a mitochondrial transit peptide in case in Table I, application no. 4, in column 6 in the same line as SEQ ID NO. 92540 is depicted, mitochondrial is mentioned; which is operable linked, too.

In another embodiment thereof the expression cassette does not comprise an additional transit peptide in case in Table I, application no. 4, in column 6 in the same line as SEQ ID NO. 92540 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 98445, or a homolog or fragment thereof, or a homolog thereof being depicted in Table I, application no. 4, column 8, in the same line as SEQ ID NO. 98445, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 98445 is depicted, plastidic is mentioned, or a mitochondrial transit peptide in case in Table I, application no. 4, in column 6 in the same line as SEQ ID NO. 98445 is depicted, mitochondrial is mentioned; which is operable linked, too.

In another embodiment thereof the expression cassette does not comprise an additional transit peptide in case in Table I, application no. 4, in column 6 in the same line as SEQ ID NO. 98445 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68413, or a homolog or fragment thereof, or a homolog thereof being depicted in Table I, application no. 4, column 8, in the same line as SEQ ID NO. 68413, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 4, in column 6 in the same line as SEQ ID NO. 68413 is depicted, plastidic is mentioned, or a mitochondrial transit peptide in case in Table I, application no. 4, in column 6 in the same line as SEQ ID NO. 68413 is depicted, mitochondrial is mentioned; which is operable linked, too.

In another embodiment thereof the expression cassette does not comprise an additional transit peptide in case in Table I, application no. 4, in column 6 in the same line as SEQ ID NO. 68413 is depicted, non-targeted is mentioned.

[0165.1.1.4] to [0170.1.1.4] for the disclosure of these paragraphs see [0165.1.1.1] to [0170.1.1.1] above.

The invention further provides a vector, comprising a nucleic acid molecule comprising 1) a nucleic acid molecule selected from the group consisting of:
(a) a nucleic acid molecule encoding a polypeptide shown in Table II, column 5 or 8, application no. 4, preferably shown in Table II A, application no. 4, in column 5, or in Table II A, application no. 4, column 8, or in Table II B, application no. 4, column 8, or a homolog or a fragment thereof;
(b) a nucleic acid molecule shown in Table I, application no. 4, column 5 or 8, preferably shown in Table I A, application no. 4, in column 5, or in Table I A, application no. 4, column 8, or in Table I B, application no. 4, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 4, preferably shown in Table II A, application no. 4, in column 5, or in Table II A, application no. 4, column 8, or in Table II B, application no. 4, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 4, preferably shown in Table I A, application no. 4, in column 5, or in Table I A, application no. 4, column 8, or in Table I B, application no. 4, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 4;
(i) a nucleic acid molecule encoding a polypeptide having the activity represented by a protein as depicted in column 5 of Table II, application no. 4;
(j) a nucleic acid molecule comprising a polynucleotide which is obtained by amplifying a cDNA library or a genomic library using the primers in Table III, application no. 4, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
   or a nucleic acid molecule comprising a sequence which is complementary thereto.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 4.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 4, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 4.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 4, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 4, in the same line as of the respective nucleic acid molecule according to 1) “plastidic” is depicted, or encoding a mitochondrial transit peptide, especially in case in column 6 of Table I, application no. 4, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 4, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 4, in the same line as of the respective nucleic acid molecule according to 1) “plastidic” is depicted, or encoding a mitochondrial transit peptide, especially in case in column 6 of Table I, application no. 4, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operable linked; a nucleic acid molecule encoding a transit peptide, especially in case in column 6 of Table I, application no. 4, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 4, in the same line as of the respective nucleic acid molecule according to 1) “plastidic” is depicted, or encoding a mitochondrial transit peptide, especially in case in column 6 of Table I, application no. 4, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 4 (in the same line).

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 4, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 4.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 4, in the same line as of the respective nucleic acid molecule according to 1) “plastidic” is depicted, or encoding a mitochondrial transit peptide, especially in case in column 6 of Table I, application no. 4, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 4, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 4, in the same line as of the respective nucleic acid molecule according to 1) “plastidic” is depicted, or encoding a mitochondrial transit peptide, especially in case in column 6 of Table I, application no. 4, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 4 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 4, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 4 (in the same line) and confers a generation or an increase of the respective fine chemical in a non-human organism or a part thereof as compared to a corresponding wild-type non-human organism or part thereof.

In a preferred embodiment of said vector said nucleic acid molecule according to 1 (a), (b), (c), (d), (e), (f), (g), (h), (i), (j) or (k), said promoter and optionally said nucleic acid molecule encoding a transit peptide are encompassed in an expression cassette. Accordingly in an embodiment the invention provides a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

The invention further provides a vector comprising a nucleic acid molecule comprising 1) a nucleic acid molecule selected from the group consisting of:
(a) a nucleic acid molecule encoding a polypeptide shown in Table II, column 5 or 8, application no. 4, preferably shown in Table II A, application no. 4, in column 5, or in Table II A, application no. 4, column 8, or in Table II B, application no. 4, column 8, or a homolog or a fragment thereof;
(b) a nucleic acid molecule shown in Table I, application no. 4, column 5 or 8, preferably shown in Table I A, application no. 4, in column 5, or in Table I A, application no. 4, column 8, or in Table I B, application no. 4, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 4, preferably shown in Table II A, application no. 4, in column 5, or in Table II A, application no. 4, column 8, or in Table II B, application no. 4, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 4, preferably shown in Table I A, application no. 4, in column 5, or in Table I A, application no. 4, column 8, or in Table I B, application no. 4, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 4;
(i) a nucleic acid molecule encoding a polypeptide having the activity represented by a protein as depicted in column 5 of Table II, application no. 4;
(j) a nucleic acid molecule comprising a polynucleotide which is obtained by amplifying a cDNA library or a genomic library using the primers in Table III, application no. 4, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
   or a nucleic acid molecule comprising a sequence which is complementary thereto;
   whereby, preferably, the nucleic acid molecule according to 1 (a), (b), (c), (d), (e), (f), (g), (h), (i), (j) or (k) distinguishes over the sequence depicted in Table I A and/or I B, application no. 4, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 4, column 5 or 8, or the coding region thereof, respectively. In another embodiment, the nucleic acid molecule of the present invention, preferably the coding region thereof, is 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 4, column 5 or 8, or the coding region thereof, respectively. In a further embodiment the nucleic acid molecule does not encode the polypeptide sequence shown in Table II A and/or II B, application no. 4, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 4, column 5 or 8 and does not encode a protein of the sequence shown in Table II A and/or II B, application no. 4, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 4, column 5 and 8, or the coding region thereof. In a further embodiment, the protein of the present invention is at least 30%,40%, 50%, 60%, 70%, 80%, 90%, 95% identical to protein sequence depicted in Table II A and/or II B, application no. 4, column 5 or 8 but less than 100%, preferably less than 99.999%, 99.99% or 99.9%, more preferably less than 99%, 985, 97%, 96% or 95% identical to the sequence shown in Table II A and/or II B, application no. 4, column 5 or 8.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 4.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 4, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 4.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 4, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 4, in the same line as of the respective nucleic acid molecule according to 1) “plastidic” is depicted, or encoding a mitochondrial transit peptide, especially in case in column 6 of Table I, application no. 4, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 4, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 4, in the same line as of the respective nucleic acid molecule according to 1) “plastidic” is depicted, or encoding a mitochondrial transit peptide, especially in case in column 6 of Table I, application no. 4, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 4, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 4, in the same line as of the respective nucleic acid molecule according to 1) “plastidic” is depicted, or encoding a mitochondrial transit peptide, especially in case in column 6 of Table I, application no. 4, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 4.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 4, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 4.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 4, in the same line as of the respective nucleic acid molecule according to 1) “plastidic” is depicted, or encoding a mitochondrial transit peptide, especially in case in column 6 of Table I, application no. 4, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 4, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 4, in the same line as of the respective nucleic acid molecule according to 1) “plastidic” is depicted, or encoding a mitochondrial transit peptide, especially in case in column 6 of Table I, application no. 4, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 4 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 4, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 4 and confers a generation or an increase of the respective fine chemical in a non-human organism or a part thereof as compared to a corresponding wild-type non-human organism or part thereof.

[0181.1.1.4] to [0209.1.1.4] for the disclosure of these paragraphs see [0181.1.1.1] to [0209.1.1.1] above.

The genes of the invention, coding for an activity selected from the group consisting of (DL)-glycerol-3-phosphatase, 2,3-dihydroxyphenylpropionate 1,2-dioxygenase, 2-C-methyl-D-erythritol 4-phosphate cytidylyltransferase, 2-oxoglutarate dehydrogenase E1 subunit, 3-phosphoglycerate dehydrogenase, 47266012-protein, 49747384_SOYBEAN-protein, ABC metal ion transporter substrate-binding protein, ABC transporter permease protein, acetolactate synthase small subunit, acetyltransferase, acyl-CoA dehydrogenase, acyl-CoA synthase, argininosuccinate lyase, aspartate aminotransferase, aspartate kinase, At1g09680-protein, At4g32480-protein, At5g16650-protein, ATP synthase subunit beta, ATP-binding component of a transport system, AX653549-protein, b1003-protein, b1108-protein, b1445-protein, b1522-protein, b1670-protein, b1930-protein, b2360-protein, b2399-protein, b2513-protein, b2613-protein, b2739-protein, b2812-protein, b3083-protein, b3442-protein, b3646-protein, b3989-protein, b4029-protein, b4056-protein, b4250-protein, beta-galactosidase, beta-hydroxylase, bifunctional aspartokinase/homoserine dehydrogenase, bifunctional purine biosynthesis protein, branched chain amino acid ABC transporter ATP-binding protein, branched chain aminotransferase, branched-chain amino acid ABC transporter permease protein, calcium-dependent protein kinase, carbamoyl-phosphate synthase subunit, CCAAT-binding transcription factor, CDP-diacylglycerol -serine O-phosphatidyltransferase, cell division control protein, cell division protein, Chaperone protein CIpB, coproporphyrinogen III oxidase, cyclin, cyclin D , D-3-phosphoglycerate dehydrogenase, dihydrolipoamide acetyltransferase, dihydroxyacid dehydratase, DNA helicase II, DnaJ-like chaperone, D-serine dehydratase, elongation factor Tu, enoyl-CoA hydratase, enterobacterial common antigen polymerase, eukaryotic translation initiation factor 5, ferredoxin, GDP-mannose dehydrogenase, geranylgeranyl pyrophosphate synthase, gibberellin 20-oxidase, glucose dehydrogenase, glucose-6-phosphate 1-dehydrogenase, glutamate-ammonia-ligase, glutaredoxin, glutathione S-transferase, glycogen (starch) synthase, glycogen synthase, glycoprotease, glycosyl transferase, heat shock protein, heat shock transcription factor, hexose transporter, histone acetyltransferase, homocitrate synthase, homoserine dehydrogenase, hydrolase, isochorismate synthase, ketol-acid reductoisomerase, kinase, L-asparaginase, major facilitator superfamily transporter protein, malate dehydrogenase, malic enzyme, membrane transport protein, metal-dependent hydrolase, methyltransferase, mitochondrial dicarboxylate transporter, monothiol glutaredoxin, monthiol glutaredoxin, murein transglycosylase, NADP-dependent malic enzyme, nitrate/nitrite transport protein, oxireductase, peptidase, permease protein of peptide ABC transporter, phosphate transporter, phosphoanhydride phosphorylase, phosphoenolpyruvate carboxykinase, phosphoribosylaminoimidazole carboxylase catalytic subunit, photosystem II protein, polyphosphate kinase, protein kinase, protein phosphatase, pyruvate carboxylase, pyruvate kinase, restriction alleviation protein, sec-independent protein translocase, sensor histidine kinase, serine protease, sll0418-protein, slr0383-protein, succinyl-CoA synthetase beta chain, thioredoxin, threonine synthase, transcription factor, transcriptional regulator, transcriptional regulator protein, transport protein, TTC0881-protein, uroporphyrin-III C-methyltransferase, valine-pyruvate transaminase, XM_{—}473199-protein, ycl027w-protein, ydr183w-protein, YEL030C-A-protein, yfl054c-protein, ygl237c-protein, yhr131c-protein, YJL127W-A-protein, YKL038W-protein, zinc finger protein, and Zm_{—}4842_BE510522-protein are also called “FCRP genes”.

[0211.1.1.4] to [0225.1.1.4] for the disclosure of these paragraphs see [0211.1.1.1] to [0225.1.1.1] above.

In addition to the sequence mentioned in Table I, application no. 4, column 5 or 8, preferably the coding region thereof, or its homologs or fragments, it may be advantageous additionally to express and/or mutate further genes in the non-human organisms. Especially advantageously, additionally at least one further gene of the biosynthetic pathway of the fine chemical is expressed in the non-human organisms such as plants or microorganisms. It is also possible that the regulation of the natural genes has been modified advantageously so that the gene and/or its gene product is no longer subject to the regulatory mechanisms which exist in the non-human organisms. This leads to an increased synthesis of the respective amino acid since, for example, feedback regulations no longer exist to the same extent or not at all. In addition it might be advantageously to combine the nucleic acids sequences of the invention containing the sequences shown in the respective line in Table I, application no. 4, column 5 or 8, preferably the coding region thereof, or homologs or fragments thereof, with genes which generally support or enhance the growth or yield of the target non-human organisms, for example genes which lead to faster growth rate of non human organism like microorganisms or plants or genes which produce stress-, pathogen-, or herbicide-resistant plants.

[0227.1.1.4] to [0239.1.1.4] for the disclosure of these paragraphs see [0227.1.1.1] to [0239.1.1.1] above.

In an embodiment of the present invention the nucleic acid construct of the present invention comprises a nucleic acid molecule as depicted in the respective line in Table I, application no. 4, column 5 or 8, preferably the coding region thereof, a fragment or a homolog thereof, and a plant promoter, such as the Big 35S promoter, the DC3 promoter, the PC Ubi promoter, the LegB4-promoter, the Super promoter, the USP promoter or the like. In another embodiment the nucleic acid construct of the present invention comprises a nucleic acid molecule as depicted in the respective line in Table I, application no. 4, column 5 or 8, preferably the coding region thereof, a fragment or a homolog thereof, and a plant promoter such as, for example, the USP, the LegB4-, the DC3 promoter or the ubiquitin promoter from parsley or other herein mentioned promoters. In a further embodiment the nucleic acid construct of the present invention comprises a nucleic acid molecule as depicted in the respective line in Table I, application no. 4, column 5 or 8, preferably the coding region thereof, a fragment or a homolog thereof, and a plant promoter, such as the Big 35S promoter, the PC Ubi promoter, the

Super promoter, the USP promoter or the like. In addition different terminators may advantageously be used in these nucleic acid constructs; examples for transcriptional termination are polyadenylation signals.

[0241.1.1.4] to [0245.1.1.4] for the disclosure of these paragraphs see [0241.1.1.1] to [0245.1.1.1] above.

Other preferred sequences for use in operable linkage in gene expression constructs are targeting sequences, which are required for targeting the gene product into specific cell compartments (for a review, see Kermode, Crit. Rev. Plant Sci. 15 (4), 285 (1996) and references cited therein), for example into the vacuole, the nucleus, all types of plastids, such as amyloplasts, chloroplasts, chromoplasts, the extracellular space, the mitochondria, the endoplasmic reticulum, elaioplasts, peroxisomes, glycosomes, and other compartments of cells or extracellular; preferred are sequences, which are involved in targeting to plastids as mentioned above. Sequences, which must be mentioned in this context are, in particular, the signal-peptide- or transit-peptide-encoding sequences which are known per se. For example, plastid-transit-peptide-encoding sequences enable the targeting of the expression product into the plastids of a plant cell. Targeting sequences are also known for eukaryotic and to a lower extent for prokaryotic organisms and can advantageously be operable linked with the nucleic acid molecule of the present invention as shown in Table I, application no. 4, columns 5 and 8, preferably the coding region thereof, or homologs or fragments thereof, and described herein to achieve an expression in one of said compartments or extracellular.

[0247.1.1.4] to [0266.1.1.4] for the disclosure of these paragraphs see [0247.1.1.1] to [0266.1.1.1] above.

An embodiment of the invention relates to a vector where the nucleic acid molecule according to the invention is linked operably to regulatory sequences which permit the expression in a prokaryotic or eukaryotic host.

A further embodiment of the invention relates to a vector in which a nucleic acid sequence encoding one of the polypeptides shown in the respective line in Table II, application no. 4, columns 5 or 8, or homologs or fragments thereof, is functionally linked to a targeting sequence such as a plastidial or mitochondrial targeting sequence. A further embodiment of the invention relates to a vector in which a nucleic acid sequence encoding one of the polypeptides shown in the respective line in Table II, application no. 4, columns 5 or 8, or homologs or fragments thereof, is functionally linked to (a) regulatory sequence which permit the expression in an organelle, such as plastids or mitochondria, preferably plastids.

A further embodiment of the invention relates to a vector which comprises a nucleic acid sequence encoding one of the polypeptides shown in the respective line in Table II, application no. 4, columns 5 or 8, or homologs or fragments thereof, but no additional targeting sequence.

[0268.1.1.4] to [0273.1.1.4] for the disclosure of these paragraphs see [0268.1.1.1] to [0273.1.1.1] above.

A further object of the invention relates to the use of a nucleic acid construct, e.g. an expression cassette, comprising one or more nucleic acid molecules encoding one or more polypeptides as depicted in the respective line(s) in Table II, application no. 4, columns 5 or 8, or homologs or fragments thereof, or comprising one or more nucleic acid molecules as depicted in the respective line(s) in Table I, application no. 4, columns 5 or 8, preferably the coding region thereof, or homologs or fragments thereof, or comprising a nucleic acid molecule hybridizing therewith for the transformation of a microorganism or a plant cell, plant tissues or a part of a plant.

In doing so, depending on the choice of promoter, the nucleic acid molecules shown in the respective line in Table I, application no. 4, column 5 or 8, preferably the coding region thereof, or homologs or fragments thereof, or the nucleic acid molecules encoding a polypeptide as depicted in the respective line in Table II, application no. 4, column 5 or 8, or homologs or fragments thereof, can be expressed in the whole plant or specifically e.g. in the leaves, in the seeds, the nodules, in roots, in the stem or other parts of the plant. Those transgenic plants overproducing sequences, e.g. as depicted in the respective line in Table I, application no. 4, column 5 or 8, preferably the coding region thereof, or homologs or fragments thereof, the plant cell, plant tissues, plants, the reproductive material thereof, or parts thereof are a further object of the present invention.

Within the framework of the present invention, the production of the threonine is due to the generation or over-expression of one or more polypeptides as depicted in the respective line(s) in Table II, application no. 4, column 5 or 8, or homologs or fragments thereof, or encoded by the corresponding nucleic acid molecules as depicted in the respective line(s) in Table I, application no. 4, column 5 or 8, preferably the coding region thereof, or homologs or fragments thereof, in the non-human organism according to the invention, advantageously in the transgenic microorganism or plant according to the invention, at least for the duration of at least one plant generation.

A constitutive expression of the polypeptide as depicted in the respective line in Table II, application no. 4, column 5 or 8, or homologs or fragments thereof, or encoded by the respective nucleic acid molecule as depicted in the respective line in Table I, application no. 4, column 5 or 8, preferably the coding region thereof, or homologs or fragments thereof, may be advantageous. On the other hand, an inducible expression may also appear desirable. Expression of the polypeptide sequences of the invention can be either directed to the cytosol or to the organelles, such as plastids or mitochondria, preferably the plastids of the host cells, preferably the plant cells, or non-targeted.

for the disclosure of this paragraph see [0278.1.1.1] above.

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 4, column 5 or 8, preferably shown in Table II A, application no. 4, column 5, or in Table II A, application no. 4, column 8, or in Table II B, application no. 4, column 8, or a homolog or a fragment thereof;
   (b) a nucleic acid molecule shown in Table I, application no. 4, column 5 or 8, preferably shown in Table I A, application no. 4, column 5, or in Table I A, application no. 4, column 8, or in Table I B, application no. 4, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 4, preferably shown in Table II A, application no. 4, column 5, or in Table II A, application no. 4, column 8, or in Table II B, application no. 4, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 4, preferably shown in Table I A, application no. 4, column 5, or in Table I A, application no. 4, column 8, or in Table I B, application no. 4, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 4;
   (i) a nucleic acid molecule encoding a polypeptide having the activity represented by a respective protein as depicted in column 5 of Table II, application no. 4;
   (j) a nucleic acid molecule comprising a polynucleotide which is obtained by amplifying a cDNA library or a genomic library using the primers in Table III, application no. 4, column 8;

and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
or a nucleic acid molecule comprising a sequence which is complementary thereto; and, if desired
2) nucleic acid molecule encoding a transit peptide, preferably encoding a plastidal transit peptide or a mitochondrial transit peptide;
   which are operable linked;]
   or comprising an expression cassette comprising
[0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 4, column 5 or 8, preferably shown in Table II A, application no. 4, column 5, or in Table II A, application no. 4, column 8, or in Table II B, application no. 4, column 8, or a homolog or a fragment thereof;
   (b) a nucleic acid molecule shown in Table I, application no. 4, column 5 or 8, preferably shown in Table I A, application no. 4, column 5, or in Table I A, application no. 4, column 8, or in Table I B, application no. 4, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 4, preferably shown in Table II A, application no. 4, column 5, or in Table II A, application no. 4, column 8, or in Table II B, application no. 4, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 4, preferably shown in Table I A, application no. 4, column 5, or in Table I A, application no. 4, column 8, or in Table I B, application no. 4, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 4;
   (i) a nucleic acid molecule encoding a polypeptide having the activity represented by a respective protein as depicted in column 5 of Table II, application no. 4;
   (j) a nucleic acid molecule comprising a polynucleotide which is obtained by amplifying a cDNA library or a genomic library using the primers in Table III, application no. 4, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]

or comprising or being transformed by a vector comprising
[1) a nucleic acid molecule selected from the group consisting of
   (a) a nucleic acid molecule encoding a polypeptide shown in Table II, application no. 4, column 5 or 8, preferably shown in Table II A, application no. 4, column 5, or in Table II A, application no. 4, column 8, or in Table II B, application no. 4, column 8, or a homolog or a fragment thereof;
   (b) a nucleic acid molecule shown in Table I, application no. 4, column 5 or 8, preferably shown in Table I A, application no. 4, column 5, or in Table I A, application no. 4, column 8, or in Table I B, application no. 4, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 4, preferably shown in Table II A, application no. 4, column 5, or in Table II A, application no. 4, column 8, or in Table II B, application no. 4, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 4, preferably shown in Table I A, application no. 4, column 5, or in Table I A, application no. 4, column 8, or in Table I B, application no. 4, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 4;
   (i) a nucleic acid molecule encoding a polypeptide having the activity represented by a respective protein as depicted in column 5 of Table II, application no. 4;
   (j) a nucleic acid molecule comprising a polynucleotide which is obtained by amplifying a cDNA library or a genomic library using the primers in Table III, application no. 4, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 4.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 4 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 4, in the same line as of the respective nucleic acid molecule according to 1) “plastidic” is depicted, or encoding a mitochondrial transit peptide, especially in case in column 6 of Table I, application no. 4, in the same line as of the respective nucleic acid molecule according to 1) “mitochondrial” is depicted.

In another embodiment of said transgenic non-human organism the expression cassette does not comprise 2) a nucleic acid molecule encoding a transit peptide, especially in case in column 6 of Table I, application no. 4, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 4.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 4, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 4, in the same line as of the respective nucleic acid molecule according to 1) “plastidic” is depicted, or encoding a mitochondrial transit peptide, especially in case in column 6 of Table I, application no. 4, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 4, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 4, in the same line as of the respective nucleic acid molecule according to 1) “plastidic” is depicted, or encoding a mitochondrial transit peptide, especially in case in column 6 of Table I, application no. 4, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 4, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 4, in the same line as of the respective nucleic acid molecule according to 1) “plastidic” is depicted, or encoding a mitochondrial transit peptide, especially in case in column 6 of Table I, application no. 4, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 4.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 4, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 4.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 4, in the same line as of the respective nucleic acid molecule according to 1) “plastidic” is depicted, or encoding a mitochondrial transit peptide, especially in case in column 6 of Table I, application no. 4, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 4, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 4, in the same line as of the respective nucleic acid molecule according to 1) “plastidic” is depicted, or encoding a mitochondrial transit peptide, especially in case in column 6 of Table I, application no. 4, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 4 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 4, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 4 (in the same line) and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

In a further embodiment the invention relates to a transgenic non-human organisms such as transgenic microorganism or transgenic plant comprising a nucleic acid molecule comprising
[1) a nucleic acid molecule selected from the group consisting of P1 (a) a nucleic acid molecule encoding a polypeptide shown in Table II, application no. 4, column 5 or 8, preferably shown in Table II A, application no. 4, column 5, or in Table II A, application no. 4, column 8, or in Table II B, application no. 4, column 8, or a homolog or a fragment thereof; P1 (b) a nucleic acid molecule shown in Table I, application no. 4, column 5 or 8, preferably shown in Table I A, application no. 4, column 5, or in Table I A, application no. 4, column 8, or in Table I B, application no. 4, column 8, preferably the coding region thereof, or a homolog or a fragment thereof; P1 (c) a nucleic acid molecule, which, as a result of the degeneracy of the genetic code, can be derived from a polypeptide sequence depicted in column 5 or 8 of Table II, application no. 4, preferably shown in Table II A, application no. 4, column 5, or in Table II A, application no. 4, column 8, or in Table II B, application no. 4, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 4, preferably shown in Table I A, application no. 4, column 5, or in Table I A, application no. 4, column 8, or in Table I B, application no. 4, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 4;
   (i) a nucleic acid molecule encoding a polypeptide having the activity represented by a respective protein as depicted in column 5 of Table II, application no. 4;
   (j) a nucleic acid molecule comprising a polynucleotide which is obtained by amplifying a cDNA library or a genomic library using the primers in Table III, application no. 4, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide; which are operable linked;]

or comprising an expression cassette comprising
[0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 4, column 5 or 8, preferably shown in Table II A, application no. 4, column 5, or in Table II A, application no. 4, column 8, or in Table II B, application no. 4, column 8, or a homolog or a fragment thereof;
   (b) a nucleic acid molecule shown in Table I, application no. 4, column 5 or 8, preferably shown in Table I A, application no. 4, column 5, or in Table I A, application no. 4, column 8, or in Table I B, application no. 4, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 4, preferably shown in Table II A, application no. 4, column 5, or in Table II A, application no. 4, column 8, or in Table II B, application no. 4, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 4, preferably shown in Table I A, application no. 4, column 5, or in Table I A, application no. 4, column 8, or in Table I B, application no. 4, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 4;
   (i) a nucleic acid molecule encoding a polypeptide having the activity represented by a respective protein as depicted in column 5 of Table II, application no. 4;
   (j) a nucleic acid molecule comprising a polynucleotide which is obtained by amplifying a cDNA library or a genomic library using the primers in Table III, application no. 4, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      and, if desired
2) nucleic acid molecule encoding a transit peptide, preferably encoding a plastidal transit peptide or a mitochondrial transit peptide;
   which are operable linked;]
   or comprising or being transformed by a vector comprising
[1) a nucleic acid molecule selected from the group consisting of
   (a) a nucleic acid molecule encoding a polypeptide shown in Table II, application no. 4, column 5 or 8, preferably shown in Table II A, application no. 4, column 5, or in Table II A, application no. 4, column 8, or in Table II B, application no. 4, column 8, or a homolog or a fragment thereof;
   (b) a nucleic acid molecule shown in Table I, application no. 4, column 5 or 8, preferably shown in Table I A, application no. 4, column 5, or in Table I A, application no. 4, column 8, or in Table I B, application no. 4, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 4, preferably shown in Table II A, application no. 4, column 5, or in Table II A, application no. 4, column 8, or in Table II B, application no. 4, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 4, preferably shown in Table I A, application no. 4, column 5, or in Table I A, application no. 4, column 8, or in Table I B, application no. 4, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 4;
   (i) a nucleic acid molecule encoding a polypeptide having the activity represented by a respective protein as depicted in column 5 of Table II, application no. 4;
   (j) a nucleic acid molecule comprising a polynucleotide which is obtained by amplifying a cDNA library or a genomic library using the primers in Table III, application no. 4, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      whereby, preferably, the nucleic acid molecule according to 1 (a), (b), (c), (d), (e), (f), (g), (h), (i), (j) or (k) distinguishes over the sequence depicted in Table I A and/or I B, application no. 4, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 4, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 4, column 5 or 8, or the coding region thereof, respectively. In a further embodiment the nucleic acid molecule does not encode the polypeptide sequence shown in Table II A and/or II B, application no. 4, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 4, column 5 or 8 and does not encode a protein of the sequence shown in Table II A and/or II B, application no. 4, column 5 or 8. Accordingly, in one embodiment, the protein encoded by a sequence of a nucleic acid according to 1 (a), (b), (c), (d), (e), (f), (g), (h), (i), (j) or (k) does not consist of the sequence shown in Table I A and/or I B, application no. 4, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 4, column 5 or 8 but less than 100%, preferably less than 99.999%, 99.99%, 99.9%, 99%, 98%, 97%, 96% or 95% identical to the sequence shown in Table II A and/or II B, application no. 4, column 5 or 8;
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 4.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 4 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 4, in the same line as of the respective nucleic acid molecule according to 1) “plastidic” is depicted, or encoding a mitochondrial transit peptide, especially in case in column 6 of Table I, application no. 4, in the same line as of the respective nucleic acid molecule according to 1) “mitochondrial” is depicted.

In another embodiment thereof the expression cassette does not comprise 2) a nucleic acid molecule encoding a transit peptide, especially in case in column 6 of Table I, application no. 4, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 4.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the same line in column 5 of Table II, application no. 4, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 4, in the same line as of the respective nucleic acid molecule according to 1) “plastidic” is depicted, or encoding a mitochondrial transit peptide, especially in case in column 6 of Table I, application no. 4, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 4, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 4, in the same line as of the respective nucleic acid molecule according to 1) “plastidic” is depicted, or encoding a mitochondrial transit peptide, especially in case in column 6 of Table I, application no. 4, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 4, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 4, in the same line as of the respective nucleic acid molecule according to 1) “plastidic” is depicted, or encoding a mitochondrial transit peptide, especially in case in column 6 of Table I, application no. 4, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 4.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 4, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 4.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 4, in the same line as of the respective nucleic acid molecule according to 1) “plastidic” is depicted, or encoding a mitochondrial transit peptide, especially in case in column 6 of Table I, application no. 4, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 4, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 4, in the same line as of the respective nucleic acid molecule according to 1) “plastidic” is depicted, or encoding a mitochondrial transit peptide, especially in case in column 6 of Table I, application no. 4, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 4 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 4, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 4 and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

[0291.1.1.4] to [0299.1.1.4] for the disclosure of these paragraphs see [0291.1.1.1] to [0299.1.1.1] above.

The term “biological active portion” or “biological activity” means a polypeptide as depicted in the respective line in Table II, application no. 4, column 3 or a portion of said polypeptide which still has at least 10% or 20%, preferably 30%, 40%, 50% or 60%, especially preferably 70%, 75%, 80%, 90% or 95% of the enzymatic or biological activity of the natural or starting enzyme or protein.

[0301.1.1.4] to [0304.1.1.4] for the disclosure of these paragraphs see [0301.1.1.1] to [0304.1.1.1] above.

Synthetic oligonucleotide primers for the amplification, e.g. as shown in the respective line in Table III, application no. 4, column 8, by means of polymerase chain reaction can be generated on the basis of a sequence shown herein, for example the sequence shown in the respective line in Table I, application no. 4, columns 5 or 8, or the sequences derived from Table II, application no. 4, columns 5 or 8, respectively.

Moreover, it is possible to identify a conserved region of a protein from various organism by carrying out protein sequence alignments with the polypeptide encoded by the nucleic acid molecules of the present invention, in particular with the sequences encoded by the nucleic acid molecule shown in the respective line in column 5 or 8 of Table I, application no. 4, from which conserved regions, and in turn, degenerate primers can be derived. Conserved regions are those, which show a very little variation in the amino acid in one particular position of several homologs from different origin. The consensus sequence and polypeptide motifs shown in the respective line in column 8 of Table IV, application no. 4, are derived from such alignments of homologous proteins.

In an embodiment of the present invention, in the method of the present invention the activity of a polypeptide comprising or consisting of a consensus sequence or at least one polypeptide motif shown in the respective line in Table IV, application no. 4, column 8, is increased. In another embodiment thereof, the method of the present invention relates to a polypeptide comprising or consisting of a consensus sequence shown in the respective line in Table IV, application no. 4, column 8 or comprising at least one polypeptide motif shown in the respective line in Table IV, application no. 4, column 8, whereby not more than 20, 15, 10, 9, 8, 7, 6, 5 4, 3, 2 or 1, or 0 of the amino acid positions indicated can be replaced by any amino acid. In another embodiment thereof, the method of the present invention relates to a polypeptide comprising or consisting of a consensus sequence shown in the respective line in Table IV, application no. 4, column 8 or comprising at least one polypeptide motif shown in the respective line in Table IV, application no. 4, column 8, whereby not more than 15%, 10%, 5%, 4%, 3%, 2% or 1%, or 0% of the amino acid positions defined as distinct specific amino acids are/is replaced by another amino acid. In another embodiment thereof, the method of the present invention relates to a polypeptide comprising or consisting of a consensus sequence shown in the respective line in Table IV, application no. 4, column 8 or comprising at least one polypeptide motif shown in the respective line in Table IV, application no. 4, column 8, whereby not more than 20, 15, 10, 9, 8, 7, 6, 5, 4, 3, 2 or 1, or 0 amino acids are inserted into a consensus sequence or polypeptide motif.

In an embodiment of the present invention, in the method of the present invention the activity of a polypeptide comprising or consisting of a consensus sequence or at least one polypeptide motif shown in the respective line in Table IV, application no. 4, column 8, is increased, whereby said polypeptide distinguishes over the sequence depicted in Table II, application no. 4, columns 5 or 8 by one or more amino acids.

In one embodiment, said polypeptide of the invention distinguishes over the sequence shown in the respective line in Table II, application no. 4, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II B, application no. 4, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in Table II, application no. 4, columns 5 or 8.

[0309.1.1.4] to [0321.1.1.4] for the disclosure of these paragraphs see [0309.1.1.1] to [0321.1.1.1] above.

Polypeptides having above-mentioned activity, i.e. conferring the production or the increased production of the fine chemical threonine as compared to a corresponding, e.g.

non-transformed, wild type non-human organism, like a microorganism or a plant cell, plant or part thereof, can be encoded by other DNA sequences which hybridize to the sequences shown in the respective line in Table I, application no. 4, columns 5 and 8, preferably the coding region thereof, at least under relaxed hybridization conditions and which encode the expression of polypeptides conferring the production or the increased production of the respective fine chemical threonine as compared to a corresponding, e.g. non-transformed, wild type non-human organism, like a microorganism or a plant cell, plant or part thereof.

[0323.1.1.4] to [0329.1.1.4] for the disclosure of this paragraph see [0323.1.1.1] to [0329.1.1.1] above.

Further, the nucleic acid molecule of the invention comprises a nucleic acid molecule, which is a complement of one of the nucleotide sequences of above-mentioned nucleic acid molecules or a portion thereof. A nucleic acid molecule or its sequence which is complementary to one of the nucleotide molecules or sequences shown in the respective line in Table I, application no. 4, columns 5 or 8, preferably the coding region thereof, homologs or fragments thereof, is one which is sufficiently complementary to one of the nucleotide molecules or sequences shown in the respective line in Table I, application no. 4, columns 5 or 8, preferably the coding region thereof, homologs or fragments thereof, such that it can hybridize to one of the nucleotide sequences shown in Table I, application no. 4, columns 5 or 8, preferably the coding region thereof, homologs or fragments thereof, thereby forming a stable duplex. Preferably, the hybridization is performed under stringent hybridization conditions. However, a complement of one of the herein disclosed sequences is preferably a sequence complement thereto according to the base pairing of nucleic acid molecules well known to the skilled person. For example, the bases A and G undergo base pairing with the bases T and U or C, resp. and visa versa. Modifications of the bases can influence the base-pairing partner.

The nucleic acid molecule of the invention comprises a nucleotide sequence which is at least about 30%, 35%, 40%, 45%, 50%, 55%, 60%, 65%, 70%, 75%, 80%, 90%, 95%, 96%, 97%, 98%, 99%, 99.5% or more homologous to a nucleotide sequence shown in Table I, application no. 4, columns 5 or 8, preferably the coding region thereof, homologs or fragments thereof, and preferably has above-mentioned activity, in particular conferring the production or the increased production of the fine chemical threonine, respectively, after increasing the activity or an activity of a gene as shown in the respective line in Table I or of a gene product, e.g. as shown in the respective line in Table II, application no. 4, column 5 or 8, by for example in one embodiment expression either in the cytosol or in an organelle such as a plastid or mitochondria or both, preferably in a plastid, or in another embodiment non-targeted or targeted.

The nucleic acid molecule of the invention comprises a nucleotide sequence or molecule which hybridizes, preferably hybridizes under stringent conditions as defined herein, to one of the nucleic acid molecule shown in the respective line in Table I, preferably Table IB, application no. 4, columns 5 or 8, preferably the coding region thereof, fragments or homologs thereof, and encodes a protein having above-mentioned activity, e.g. conferring the production or the increased production of the respective fine chemical threonine as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted, and optionally, the activity thereof is selected from the group consisting of (DL)-glycerol-3-phosphatase, 2,3-dihydroxyphenylpropionate 1,2-dioxygenase, 2-C-methyl-D-erythritol 4-phosphate cytidylyltransferase, 2-oxoglutarate dehydrogenase E1 subunit, 3-phosphoglycerate dehydrogenase, 47266012-protein, 49747384_SOYBEAN-protein, ABC metal ion transporter substrate-binding protein, ABC transporter permease protein, acetolactate synthase small subunit, acetyltransferase, acyl-CoA dehydrogenase, acyl-CoA synthase, argininosuccinate lyase, aspartate aminotransferase, aspartate kinase, At1g09680-protein, At4g32480-protein, At5g16650-protein, ATP synthase subunit beta, ATP-binding component of a transport system, AX653549-protein,b1003-protein, b1108-protein, b1445-protein,b1522-protein,b1670-protein,b1930-protein, b2360-protein, b2399-protein, b2513-protein, b2613-protein, b2739-protein, b2812-protein, b3083-protein, b3442-protein, b3646-protein, b3989-protein, b4029-protein, b4056-protein, b4250-protein, beta-galactosidase, beta-hydroxylase, bifunctional aspartokinase/homoserine dehydrogenase, bifunctional purine biosynthesis protein, branched chain amino acid ABC transporter ATP-binding protein, branched chain aminotransferase, branched-chain amino acid ABC transporter permease protein, calcium-dependent protein kinase, carbamoyl-phosphate synthase subunit, CCAAT-binding transcription factor, CDP-diacylglycerol-serine O-phosphatidyltransferase, cell division control protein, cell division protein, Chaperone protein CIpB, coproporphyrinogen III oxidase, cyclin, cyclin D , D-3-phosphoglycerate dehydrogenase, dihydrolipoamide acetyltransferase, dihydroxyacid dehydratase, DNA helicase II, DnaJ-like chaperone, D-serine dehydratase, elongation factor Tu, enoyl-CoA hydratase, enterobacterial common antigen polymerase, eukaryotic translation initiation factor 5, ferredoxin, GDP-mannose dehydrogenase, geranylgeranyl pyrophosphate synthase, gibberellin 20-oxidase, glucose dehydrogenase, glucose-6-phosphate 1-dehydrogenase, glutamate-ammonia-ligase, glutaredoxin, glutathione S-transferase, glycogen (starch) synthase, glycogen synthase, glycoprotease, glycosyl transferase, heat shock protein, heat shock transcription factor, hexose transporter, histone acetyltransferase, homocitrate synthase, homoserine dehydrogenase, hydrolase, isochorismate synthase, ketol-acid reductoisomerase, kinase, L-asparaginase, major facilitator superfamily transporter protein, malate dehydrogenase, malic enzyme, membrane transport protein, metaldependent hydrolase, methyltransferase, mitochondrial dicarboxylate transporter, monothiol glutaredoxin, monthiol glutaredoxin, murein transglycosylase, NADP-dependent malic enzyme, nitrate/nitrite transport protein, oxireductase, peptidase, permease protein of peptide ABC transporter, phosphate transporter, phosphoanhydride phosphorylase, phosphoenolpyruvate carboxykinase, phosphoribosylaminoimidazole carboxylase catalytic subunit, photosystem II protein, polyphosphate kinase, protein kinase, protein phosphatase, pyruvate carboxylase, pyruvate kinase, restriction alleviation protein, sec-independent protein translocase, sensor histidine kinase, serine protease, sll0418-protein, slr0383-protein, succinyl-CoA synthetase beta chain, thioredoxin, threonine synthase, transcription factor, transcriptional regulator, transcriptional regulator protein, transport protein, TTC0881-protein, uroporphyrin-III C-methyltransferase, valine-pyruvate transaminase, XM_{—}473199-protein, ycl027w-protein, ydr183w-protein, YEL030C-A-protein, yfl054c-protein, ygl237c-protein, yhr131c-protein, YJL127W-A-protein, YKL038W-protein, zinc finger protein, and Zm_{—}4842_BE510522-protein, respectively.

Moreover, the nucleic acid molecule of the invention can comprise only a portion of the coding region of one of the nucleic acid molecules depicted in Table I, application no.

4, columns 5 or 8, for example a fragment which can be used as a probe or primer or a fragment encoding a biologically active portion of the polypeptide of the present invention or of a polypeptide used in the process of the present invention, i.e. having above-mentioned activity, e.g. conferring the production or the increased production of the respective fine chemical threonine as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted. The nucleotide sequences determined from the cloning of the present protein-according-to-the-inventionencoding gene allows for the generation of probes and primers designed for use in identifying and/or cloning its homologues from other cell types and organisms. The probe/primer typically comprises a substantially purified oligonucleotide. The oligonucleotide typically comprises a region of nucleotide sequence that hybridizes under stringent conditions to at least about 12, 15 preferably about 20 or 25, more preferably about 40, 50 or 75 consecutive nucleotides of a sense strand of one of the sequences set forth, e.g., in the respective line in Table I, application no. 4, column 5 or 8, preferably the coding region thereof, or a homolog or a fragment thereof, an anti-sense sequence of one of the sequences, e.g., set forth in the respective line in Table I, application no. 4, columns 5 or 8, preferably the coding region thereof, or a homolog or a fragment thereof or naturally occurring mutants thereof. Primers based on a nucleic acid molecule of invention can be used in PCR reactions to clone homologs of the polypeptide of the invention or of the polypeptide used in the process of the invention, e.g. as the primers described in the examples of the present invention, e.g. as shown in the examples. A PCR with the primers shown in the respective line in Table III, column 8 will result in a fragment of the gene product as shown in Table II, application no. 4, column 5.

Primer sets are interchangeable. The person skilled in the art knows how to design and combine said primers to result in the desired product, e.g. in a full length clone or a partial sequence. Probes based on the sequences of the nucleic acid molecule of the invention or used in the process of the present invention can be used to detect transcripts or genomic sequences encoding the same or homologous proteins. The probe can further comprise a label group attached thereto, e.g. the label group can be a radioisotope, a fluorescent compound, an enzyme, or an enzyme co-factor. Such probes can be used as a part of a genomic marker test kit for identifying cells which express a polypeptide of the invention or used in the process of the present invention, such as by measuring a level of an encoding nucleic acid molecule in a sample of cells, e.g. detecting mRNA levels or determining, whether a genomic gene comprising the sequence of the polynucleotide of the invention or used in the processes of the present invention has been mutated or deleted.

As the expression of a respective nucleic acids of the invention is related to the synthesis of the respective fine chemical threonine its function as a probe extends to the detection of microorganisms, plant tissues, plants, plant variets, plant ecotypes or plant genera with varying capability or potential for synthesis of the respective fine chemical threonine. Therefore in one embodiment the present invention relates to a method for analyzing the capability or potential of a plant tissue, a plant, a plant variety or plant ecotype to produce the fine chemical threonine by using the nucleic acid of the invention or parts thereof as a probe to detect the amount of the nucleic acid of the invention in the non-human organism or a part thereof in comparison to another non-human organism.

The nucleic acid molecule of the invention encodes a polypeptide or portion thereof which includes an amino acid sequence which is sufficiently homologous to the amino acid sequence shown in respective line in Table II, application no. 4, columns 5 or 8 such that the protein or portion thereof maintains the ability to confer the production or the increased production of the respective fine chemical threonine as compared to a corresponding, e.g. non-transformed, wild type non-human organism, like a microorganism or a plant cell, plant or part thereof, in particular increasing the activity as mentioned above or as described in the examples in microorganisms or plants.

As used herein, the language “sufficiently homologous” refers to proteins or portions thereof which have amino acid sequences which include a minimum number of identical or equivalent amino acids (e.g. an amino acid which has a similar side chain as an amino acid in one of the sequences of the polypeptide of the present invention) to an amino acid sequence shown in the respective line in Table II, application no. 4, columns 5 or 8 such that the protein or portion thereof is able to confer the production or the increased production of the respective fine chemical threonine as compared to a corresponding, e.g. non-transformed, wild type non-human organism, like a microorganism or a plant cell, plant or part thereof. For example having the activity of a protein as shown in the respective line in Table II, application no. 4, column 3 and as described herein.

In an embodiment of the present invention, the nucleic acid molecule of the present invention comprises a nucleic acid molecule that encodes a portion of the protein of the present invention. The protein is at least about 30%, 35%, 40%, 45% 50%, 55%, 60%, 65%, 70%, 75%, 80%, 85%, 90%, 91%, 92%, 93%, 94%, 95%, 96%, 97%, 98%, 99%, 99.5% or more homologous to an entire amino acid sequence shown in the respective line of Table II, application no. 4, column 5 or 8 and has the above-mentioned activity, e.g. conferring the production or the increased production of the respective fine chemical threonine as compared to a corresponding, e.g. non-transformed, wild type non-human organism, like a microorganism or a plant cell, plant or part thereof by, for example in a embodiment expression either in the cytosol or in an organelle such as a plastid or mitochondria or both, preferably in a plastid, or in another embodiment by targeted or non-targeted expression.

[0338.1.1.4] to [0339.1.1.4] for the disclosure of these paragraphs see [0338.1.1.1] to [0339.1.1.1] above.

The invention further relates to nucleic acid molecules or methods using said nucleic acid molecules that differ from one of the nucleotide sequences shown in the respective line in Table I, application no. 4, columns 5 or 8, preferably the coding region thereof, or fragments or homologs thereof, due to degeneracy of the genetic code but encode a polypeptide of the present invention, in particular a polypeptide having the above-mentioned activity, e.g. as those polypeptides depicted in the respective line in Table II, application no. 4, columns 5 or 8, fragments or homologs thereof. Advantageously, the nucleic acid molecule of the invention comprises, or in an other embodiment has, a nucleotide sequence encoding a protein comprising, or in an other embodiment having, an amino acid sequence shown in the respective line in Table II, application no. 4, columns 5 or 8, fragments or homologs thereof. In a further embodiment, the nucleic acid molecule of the invention encodes a full length protein which is substantially homologous to an amino acid sequence shown in the respective line in Table II, application no. 4, columns 5 or 8, or homologs thereof. However, in an embodiment, the nucleic acid molecule of the present invention does not consist of the sequence shown in the respective line in Table I, preferably Table IA, application no. 4, columns 5 or 8, preferably the coding region thereof, but the functional homologs thereof.

[0341.1.1.4] to [0343.1.1.4] for the disclosure of this paragraph see [0341.1.1.1] to [0343.1.1.1] above.

Accordingly, in another embodiment, a nucleic acid molecule of the invention is at least 15, 20, 25 or 30 nucleotides in length. Preferably, it hybridizes under stringent conditions to a nucleic acid molecule comprising a nucleotide sequence of the nucleic acid molecule of the present invention or used in the process of the present invention, e.g. comprising the sequence shown in the respective line in Table I, application no. 4, columns 5 or 8, preferably the coding region thereof. In another embodiment the nucleic acid molecule is preferably at least 20, 30, 50, 100, 250 or more nucleotides in length.

for the disclosure of this paragraph see [0345.1.1.1] above.

Preferably, a nucleic acid molecule of the invention that hybridizes under stringent conditions to a sequence shown in the respective line in Table I, application no. 4, columns 5 or 8, preferably the coding region thereof, corresponds to a naturally-occurring nucleic acid molecule of the invention. As used herein, a “naturally-occurring” nucleic acid molecule refers to a RNA or DNA molecule having a nucleotide sequence that occurs in nature (e.g. encodes a natural protein). Preferably, the nucleic acid molecule encodes a natural protein having above-mentioned activity, e.g. conferring the production or the increased production of the respective fine chemical threonine as compared to a corresponding, e.g. non-transformed, wild type non-human organism, like a microorganism or a plant cell, plant or part thereof after increasing the expression or activity thereof or the activity of a protein of the invention or used in the process of the invention, in an embodiment for example expression either in the cytosol or in an organelle such as a plastid or mitochondria, preferably in plastids, or, in another embodiment by targeted or non-targeted expression.

for the disclosure of this paragraph see [0347.1.1.1] above.

For example, nucleotide substitutions leading to amino acid substitutions at “non-essential” amino acid residues can be made in a sequence of the nucleic acid molecule of the invention or used in the process of the invention, e.g. shown in the respective line in Table I, application no. 4, columns 5 or 8, preferably the coding region thereof, or fragments or homologs thereof.

[0349.1.1.4] to [0350.1.1.4] for the disclosure of these paragraphs see [0349.1.1.1] to [0350.1.1.1] above.

Accordingly, the invention relates to nucleic acid molecules encoding a polypeptide having above-mentioned activity, in a non-human organisms or parts thereof by for example expression either in the cytosol or in an organelle such as a plastid or mitochondria or both, preferably in plastids, that contain changes in amino acid residues that are not essential for said activity. Such polypeptides differ in amino acid sequence from a sequence depicted in the respective line in Table II, especially Table IIA, application no. 4, columns 5 or 8, but retain said activity described herein. The nucleic acid molecule can comprise a nucleotide sequence encoding a polypeptide, wherein the polypeptide comprises an amino acid sequence at least about 50% identical to an amino acid sequence shown in the respective line in Table II, application no. 4, columns 5 or 8, and is capable of conferring the production or the increased production of the respective fine chemical threonine as compared to a corresponding, e.g. non-transformed, wild type non-human organism, like a microorganism or a plant cell, plant or part thereof, after increasing its activity for example in an embodiment by expression either in the cytosol or in an organelle such as a plastid or mitochondria or both, preferably in plastids, or, in another embodiment by targeted or non-targeted expression. Preferably, the protein encoded by the nucleic acid molecule is at least about 60%, 70%, 80%, 90%, 95%, 96%, 97%, 98%, 99% or 99.5% identical to the sequence shown in the respective line in Table II, application no. 4, columns 5 or 8.

8 0352.1.1.4] to [0357.1.1.4] for the disclosure of these paragraphs see [0352.1.1.1] to [0357.1.1.1] above.

Functional equivalents of the nucleic acid molecules according to the present invention and/or used in the process according to the present invention represent an embodiment of homologs. Functional equivalents derived from the nucleic acid molecules as shown in the respective line in Table I, application no. 4, columns 5 or 8, preferably the coding region thereof, according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the nucleic acid molecules as shown in the respective line in Table I, application no. 4, columns 5 or 8, preferably the coding region thereof, according to the invention and encode polypeptides having essentially the same properties as the polypeptide as shown in the respective line in Table II, application no. 4, columns 5 or 8.

Functional equivalents derived from one of the polypeptides as shown in the respective line in Table II, application no. 4, columns 5 or 8 according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the polypeptides as shown in the respective line in Table II, application no. 4, columns 5 or 8 according to the invention and having essentially the same properties as the polypeptide as shown in the respective line in Table II, application no. 4, columns 5 or 8.

for the disclosure of this paragraph see [0359.1.1.1] above.

A nucleic acid molecule encoding a homolog to a polypeptide depicted in the respective line in Table II, application no. 4, columns 5 or 8 can be created by introducing one or more nucleotide substitutions, additions or deletions into the nucleic acid molecule of the present invention, in particular of Table I, application no. 4, columns 5 or 8, in the respective line, such that one or more amino acid substitutions, additions or deletions are introduced into the encoded protein. Mutations can be introduced into the nucleic acid molecules as depicted in the respective line in Table I, application no. 4, columns 5 or 8, preferably the coding region thereof, by standard techniques, such as site-directed mutagenesis, PCR-mediated mutagenesis or other methods known to the person skilled in the art.

[0361.1.1.4] to [0363.1.1.4] for the disclosure of these paragraphs see [0361.1.1.1] to [0363.1.1.1] above.

Homologues of the nucleic acid sequences used, with the sequences shown in the respective line in Table I, application no. 4, columns 5 or 8, preferably the coding region thereof, comprise also allelic variants with at least approximately 30%, 35%, 40%, 45%, 50%, 55%, 60%, 65%, 70%, 75%, 80%, 85%, 90%, 91%, 92%, 93%, 94% 95%. 96%, 97%, 98%, 99%, 99.5% or more % homology with one of the nucleotide sequences shown or the above-mentioned derived nucleic acid sequences or their homologues or derivatives or parts of these.

Allelic variants encompass in particular functional variants which can be obtained by deletion, insertion or substitution of nucleotides from the sequences shown in the respective line, preferably in Table I, columns 5 or 8, preferably the coding region thereof, or from the derived nucleic acid sequences, the intention being, however, that the enzymatic activity or the biological activity of the resulting proteins synthesized is advantageously retained or increased.

In one embodiment of the present invention, the nucleic acid molecule of the invention or used in the process of the invention comprises the sequences shown in the respective line in any of the Table I, application no. 4, columns 5 or 8, preferably the coding region thereof, fragments or homologs thereof. It is preferred that the nucleic acid molecule comprises as little as possible other nucleotides not shown in the respective line in any one of Table I, application no. 4, columns 5 or 8, preferably the coding region thereof, fragments or homologs thereof. In one embodiment, the nucleic acid molecule comprises less than 500, 400, 300, 200, 100, 90, 80, 70, 60, 50 or 40 further nucleotides. In a further embodiment, the nucleic acid molecule comprises less than 30, 20 or 10 further nucleotides. In one embodiment, the nucleic acid molecule used in the process of the invention is identical to the sequences shown in the respective line in Table I, application no. 4, columns 5 or 8, preferably the coding region thereof.

Also preferred is that the nucleic acid molecule used in the process of the invention encodes a polypeptide comprising the sequence shown in the respective line in Table II, application no. 4, columns 5 or 8, fragments or homologs thereof. In one embodiment, the nucleic acid molecule encodes less than 150, 130, 100, 80, 60, 50, 40 or 30 further amino acids. In a further embodiment, the encoded polypeptide comprises less than 20, 15, 10, 9, 8, 7, 6 or 5 further amino acids. In one embodiment used in the inventive process, the encoded polypeptide is identical to the sequences shown in the respective line in Table II, application no. 4, columns 5 or 8.

In one embodiment, the nucleic acid molecule of the invention or used in the process encodes a polypeptide comprising the sequence shown in the respective line in Table II, application no. 4, columns 5 or 8, or fragments or homologs thereof, and comprises less than 100 further nucleotides. In a further embodiment, said nucleic acid molecule comprises less than 30 further nucleotides. In one embodiment, the nucleic acid molecule used in the process is identical to a coding sequence of the sequences shown in the respective line in Table I, application no. 4, columns 5 or 8.

Polypeptides (=proteins), which still have the essential biological or enzymatic activity of the polypeptide of the present invention conferring the production or the increased production of the respective fine chemical threonine as compared to a corresponding, e.g. non-transformed, wild type non-human organism, like a microorganism or a plant cell, plant or part thereof, i.e. whose activity is essentially not reduced, are polypeptides with at least 10%, 20%, 30%, 40%, 50%, 60%, 70%, 80%, 90%, 95% or more of the wild type biological activity or enzymatic activity, advantageously, the activity is essentially not reduced in comparison with the activity of a polypeptide shown in the respective line in Table II, application no. 4, columns 5 or 8 expressed under identical conditions.

Homologues of nucleic acid molecules shown in the respective line in Table I, application no. 4, columns 5 or 8, preferably the coding region thereof, or fragments thereof, or of the derived sequences shown in the respective line in Table II, application no. 4, columns 5 or 8, or fragments thereof, also mean truncated sequences, cDNA, single-stranded DNA or RNA of the coding and noncoding DNA sequence. Homologues of said sequences are also understood as meaning derivatives, which comprise noncoding regions such as, for example, UTRs, introns, terminators, enhancers or promoter variants. The promoters upstream of the nucleotide sequences stated can be modified by one or more nucleotide substitution(s), insertion(s) and/or deletion(s) without, however, interfering with the functionality or activity either of the promoters, the open reading frame (=ORF) or with the 3′-regulatory region such as terminators or other 3′-regulatory regions, which are far away from the ORF. It is furthermore possible that the activity of the promoters is increased by modification of their sequence, or that they are replaced completely by more active promoters, even promoters from heterologous organisms. Appropriate promoters are known to the person skilled in the art and are mentioned herein below.

[0370.1.1.4] to [0379.1.1.4] for the disclosure of these paragraphs see [0370.1.1.1] [0379.1.1.1] above.

Moreover, a native polypeptide conferring the increase of the respective fine chemical threonine in a non-human organism or a part thereof can be isolated from cells (e.g., endothelial cells), for example using the antibody of the present invention as described below, in particular, an antibody against proteins having (DL)-glycerol-3-phosphatase, 2,3-dihydroxyphenylpropionate 1,2-dioxygenase, 2-C-methyl-D-erythritol 4-phosphate cytidylyltransferase, 2-oxoglutarate dehydrogenase E1 subunit, 3-phosphoglycerate dehydrogenase, 47266012-protein, 49747384_SOYBEAN-protein, ABC metal ion transporter substrate-binding protein, ABC transporter permease protein, acetolactate synthase small subunit, acetyltransferase, acyl-CoA dehydrogenase, acyl-CoA synthase, argininosuccinate lyase, aspartate aminotransferase, aspartate kinase, At1g09680-protein, At4g32480-protein, At5g16650-protein, ATP synthase subunit beta, ATP-binding component of a transport system, AX653549-protein, b1003-protein, b1108-protein, b1445-protein, b1522-protein, b1670-protein, b1930-protein, b2360-protein, b2399-protein, b2513-protein, b2613-protein, b2739-protein, b2812-protein, b3083-protein, b3442-protein, b3646-protein, b3989-protein, b4029-protein, b4056-protein, b4250-protein, beta-galactosidase, beta-hydroxylase, bifunctional aspartokinase/homoserine dehydrogenase, bifunctional purine biosynthesis protein, branched chain amino acid ABC transporter ATP-binding protein, branched chain aminotransferase, branched-chain amino acid ABC transporter permease protein, calcium-dependent protein kinase, carbamoyl-phosphate synthase subunit, CCAAT-binding transcription factor, CDP-diacylglycerol-serine O-phosphatidyltransferase, cell division control protein, cell division protein, Chaperone protein CIpB, coproporphyrinogen III oxidase, cyclin, cyclin D , D-3-phosphoglycerate dehydrogenase, dihydrolipoamide acetyltransferase, dihydroxyacid dehydratase, DNA helicase II, DnaJ-like chaperone, D-serine dehydratase, elongation factor Tu, enoyl-CoA hydratase, enterobacterial common antigen polymerase, eukaryotic translation initiation factor 5, ferredoxin, GDP-mannose dehydrogenase, geranylgeranyl pyrophosphate synthase, gibberellin 20-oxidase, glucose dehydrogenase, glucose-6-phosphate 1-dehydrogenase, glutamate-ammonia-ligase, glutaredoxin, glutathione S-transferase, glycogen (starch) synthase, glycogen synthase, glycoprotease, glycosyl transferase, heat shock protein, heat shock transcription factor, hexose transporter, histone acetyltransferase, homocitrate synthase, homoserine dehydrogenase, hydrolase, isochorismate synthase, ketol-acid reductoisomerase, kinase, L-asparaginase, major facilitator superfamily transporter protein, malate dehydrogenase, malic enzyme, membrane transport protein, metaldependent hydrolase, methyltransferase, mitochondrial dicarboxylate transporter, monothiol glutaredoxin, monthiol glutaredoxin, murein transglycosylase, NADP-dependent malic enzyme, nitrate/nitrite transport protein, oxireductase, peptidase, permease protein of peptide ABC transporter, phosphate transporter, phosphoanhydride phosphorylase, phosphoenolpyruvate carboxykinase, phosphoribosylaminoimidazole carboxylase catalytic subunit, photosystem II protein, polyphosphate kinase, protein kinase, protein phosphatase, pyruvate carboxylase, pyruvate kinase, restriction alleviation protein, sec-independent protein translocase, sensor histidine kinase, serine protease, sll0418-protein, slr0383-protein, succinyl-CoA synthetase beta chain, thioredoxin, threonine synthase, transcription factor, transcriptional regulator, transcriptional regulator protein, transport protein, TTC0881-protein, uroporphyrin-III C-methyltransferase, valine-pyruvate transaminase, XM_{—}473199-protein, ycl027w-protein, ydr183w-protein, YEL030C-A-protein, yfl054c-protein, ygl237c-protein, yhr131c-protein, YJL127W-A-protein, YKL038W-protein, zinc finger protein, or Zm_{—}4842_BE510522-protein activity, respectively, or an antibody against polypeptides as shown in the respective line in Table II, application no. 4, columns 5 or 8, or fragments or homologs thereof which can be produced by standard techniques utilizing the polypeptid of the present invention or fragment thereof, i.e., the polypeptide of this invention (FCRP). Preferred are monoclonal antibodies.

for the disclosure of this paragraph see [0381.1.1.1] above.

In one embodiment, the present invention relates to a polypeptide as depicted in the respective line in Table II, especially in Table IIA or especially in Table IIB, application no. 4, columns 5 or 8, or fragment or homolog thereof or as coded by the nucleic acid molecule shown in the respective line in Table I, especially Table IA or especially Table IB, application no. 4, columns 5 or 8, preferably the coding region thereof, or fragment or homolog thereof.

In an embodiment, the present invention relates to a polypeptide comprising or consisting of a consensus sequence shown in the respective line in Table IV, application no. 4, column 8 or at least one polypeptide motif shown in the respective line in Table IV, application no. 4, column 8.

In an embodiment thereof, the present invention relates to a polypeptide comprising or consisting of a consensus sequence shown in the respective line in Table IV, application no. 4, column 8 or comprising at least one polypeptide motif shown in the respective line in Table IV, application no. 4, column 8, whereby not more than 20, 15, 10, 9, 8, 7, 6, 5, 4, 3, 2, or 1, or 0 of the amino acids can be replaced by any amino acid.

In another embodiment thereof, the present invention relates to a polypeptide comprising or consisting of a consensus sequence shown in the respective line in Table IV, application no. 4, column 8 or comprising at least one polypeptide motif shown in the respective line in Table IV, application no. 4, column 8, whereby not more than 15%, 10%, 5%, 4%, 3%, 2% or1% or 0% of the amino acids defined as distinct specific amino acids are/is replaced by another amino acid.

In another embodiment thereof, the present invention relates to a polypeptide comprising or consisting of a consensus sequence shown in the respective line in Table IV, application no. 4, column 8 or comprising at least one polypeptide motif shown in the respective line in Table IV, application no. 4, column 8, whereby not more than 20, 15, 10, 9, 8, 7, 6, 5, 4, 3, 2, or 1, or 0 amino acids are inserted into the consensus sequence or polypeptide motif.

In an embodiment of the present invention, the polypeptide of the present invention comprises or consists of a consensus sequence or at least one polypeptide motif shown in the respective line in Table IV, application no. 4, column 8, whereby said polypeptide distinguishes over the sequence depicted in the respective line in Table II, application no. 4, columns 5 or 8 by one or more amino acids. In one embodiment, said polypeptide of the invention distinguishes over the sequence shown in the respective line in Table II, application no. 4, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 4, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in the respective line in Table II, application no. 4, columns 5 and 8.

In an embodiment, the invention relates to a polypeptide conferring an increase in the respective fine chemical threonine in a non-human organism, especially a microorganism or a plant, or a part thereof, being encoded by the nucleic acid molecule of the invention or used in the process of the invention and having a sequence which distinguishes over the sequence as shown in the respective line in Table II, application no. 4, columns 5 or 8 by one or more amino acids (but not exceeding 5%, preferably 4%, 3%, 2%, 1%, 0.5%, 0.2% or 0.1%). In an embodiment, said polypeptide of the invention does not comprise or consist of the sequence shown in the respective line in Table II, application no. 4, columns 5 or 8. In an embodiment, said polypeptide of the present invention is less than 100%, 99.999%, 99.99%, 99.9% or 99% identical. In one embodiment, said polypeptide which differs at least in one or more amino acids (but not exceeding 5%, preferably 4%, 3%, 2%, 1%, 0.5%, 0.2% or 0.1%) from the polypeptide shown in the respective line in Table II, application no. 4, columns 5 and 8 does not comprise a protein of the sequence shown in the respective line in Table II A and/or II B, application no. 4, columns 5 or 8.

In an embodiment, the present invention relates to a polypeptide having the activity of the protein as shown in the respective line in Table II, application no. 4, column 3, which distinguishes over the sequence depicted in the respective line in Table II, application no. 4, columns 5 or 8 by one or more amino acids, preferably by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 4, columns 5 and 8 by not more than 80%, 70%, 60%, 50%, 40%, 30%, 20%, 10%, 5%, 4%, 3%, 2%, 1% or 0.5%.

In another embodiment the polypeptide of the invention takes the form of a preprotein consisting of a plastidial or mitochondrial transit peptide joint to a polypeptide having the activity of the protein as shown in Table II, column 3, from which the transit peptide is preferably cleaved off upon transport of the preprotein into the organelle for example into the plastid or mitochondrion.

In another embodiment the polypeptide of the invention takes not the form of a preprotein.

[0390.1.1.4] to [0391.1.1.4] for the disclosure of these paragraphs see [0390.1.1.1] to [0391.1.1.1] above.

A polypeptide of the invention can participate in the process of the present invention. The polypeptide or a portion thereof comprises preferably an amino acid sequence, which is sufficiently homologous to an amino acid sequence shown in the respective line in Table II A and/or II B, application no. 4, columns 5 or 8.

Further, the polypeptide can have an amino acid sequence which is encoded by a nucleotide sequence which hybridizes, preferably hybridizes under stringent conditions as described above, to a nucleotide sequence of the nucleic acid molecule of the present invention. Accordingly, the polypeptide has an amino acid sequence which is encoded by a nucleotide sequence that is at least about 35%, 40%, 45%, 50%, 55%, 60%, 65%, 70%, 75%, 80%, 85%, 90%, 91%, 92%, 93%, 94%, 95%, 96%, 97%, 98%, 99%, 99.5% or more homologous to one of the amino acid sequences as shown in the respective line in Table II A and/or II B, application no. 4, columns 5 or 8, or fragments thereof. The preferred polypeptide of the present invention preferably possesses at least one of the activities according to the invention and described herein. A preferred polypeptide of the present invention includes an amino acid sequence encoded by a nucleotide sequence which hybridizes, preferably hybridizes under stringent conditions, to a nucleotide sequence shown in the respective line in Table I A and/or I B, application no. 4, columns 5 or 8, preferably the coding region thereof, or fragments thereto or which is homologous thereto, as defined above.

The polypeptide of the present invention can vary from the sequences shown in the respective line in Table II A and/or II B, application no. 4, columns 5 or 8 in amino acid sequence due to natural variation or mutagenesis, as described in detail herein. Accordingly, the polypeptide comprise an amino acid sequence which is at least about 35%, 40%, 45%, 50%, 55%, 60%, 65%, 70%, 75%, 80%, 85% or 90, 91%, 92%, 93%, 94%, 95%, 96%, 97%, 98%, 99%, 99.5% or more homologous to an entire amino acid sequence shown in the respective line in Table II A and/or II B, application no. 4, columns 5 or 8.

Biologically active portions of a polypeptide of the present invention include peptides comprising amino acid sequences derived from the amino acid sequence of the polypeptide of the present invention or used in the process of the present invention, e.g., the amino acid sequence shown in the respective line in Table II, application no. 4, columns 5 or 8 or the amino acid sequence of a protein homologous thereto, which include fewer amino acids than a full length polypeptide of the present invention or used in the process of the present invention or the full length protein which is homologous to a polypeptide of the present invention or used in the process of the present invention depicted herein, and exhibit at least one activity of a polypeptide of the present invention or used in the process of the present invention.

for the disclosure of this paragraph see [0396.1.1.1] above.

Manipulation of the nucleic acid molecule of the invention may result in the production of a protein having differences from the protein as shown in the respective line in Table II, application no. 4, column 5 or 8. Differences shall mean at least one amino acid different from the sequences as shown in the respective line in Table II, application no. 4, column 5 or 8, preferably at least 2, 3, 4, 5, 6, 7, 8, 9 or 10 amino acids (especially but not exceeding 0.1%, preferably 0.2%, 0.5%, 1%, 2%, 3% 4% or 5%), more preferably at least 15, 20, 25, 30, 35, 40, 45 or 50 amino acids (especially but not exceeding 0.5%, preferably 1%, 2%, 3% 4%, 5%, 10% or 25%) different from the sequences as shown in the respective line in Table II, application no. 4, column 5 or 8. These proteins may be improved in efficiency or activity, or may be more stable and therefore present in greater numbers in the cell as compared to the wild-type cell.

Any mutagenesis strategies for the polypeptide of the present invention or the polypeptide used in the process of the present invention to result in increasing said activity are not meant to be limiting; variations on these strategies will be readily apparent to one skilled in the art. Using such strategies, and incorporating the mechanisms disclosed herein, the nucleic acid molecule and polypeptide of the invention may be utilized to generate algae, ciliates, fungi, other microorganisms like C. glutamicum or plants or parts thereof, expressing proteins as shown in the respective line in Table II, application no. 4, column 5 or 8, or mutated proteins thereof. The nucleic acid molecules and polypeptide molecules of the invention are expressed such that the yield, production, and/or efficiency of production of a desired compound is improved.

[0399.1.1.4] to [0401.1.1.4] for the disclosure of this paragraph see [0399.1.1.1] to [0401.1.1.1] above.

In one embodiment, a protein (=polypeptide) as shown in the respective line in Table II, application no. 4, column 5 or 8 refers to a polypeptide having an amino acid sequence corresponding to the polypeptide of the invention, especially to a polypeptide as shown in Table II, application no. 4, columns 5 or 8 or homologs thereof, or used in the process of the invention, whereas a “non-inventive protein or polypeptide” or “other polypeptide” refers to a polypeptide having an amino acid sequence corresponding to a protein which is not substantially homologous to a polypeptide of the invention, preferably which is not substantially homologous to a polypeptide or protein as shown in the respective line in Table II, application no. 4, column 5 or 8 or homologs thereof, e.g., a protein which does not confer the activity described herein and which is derived from the same or a different organism.

[0403.1.1.4] to [0409.1.1.4] for the disclosure of these paragraphs see [0403.1.1.1] to [0409.1.1.1] above.

As the nucleic acid molecules of the invention and/or the expression thereof is related to the synthesis of the respective fine chemical threonine its function as a probe extends to the detection of microorganisms, plant tissues, plants, plant variets, plant ecotypes or plant genera with varying, advantageously increased, capability or potential for synthesis of the respective fine chemical threonine. Therefore in one embodiment the present invention relates to a method for analyzing the capability or potential of a plant tissue, a plant, a plant variety or ecotype to produce the respective fine chemical threonine by using the respective antibody of the invention as a probe to detect the amount of the polypeptide encoded by said nucleic acid molecule of the invention in a non-human organism in comparison to another organism.

Therefore in one embodiment the invention relates to a method to probe a non-human organism for its capability for the production of the respective fine chemical comprising the steps of
a) analyzing the sequence and/or the expression of the respective nucleic acid molecule of the invention in least two non-human organisms which differ in the capability to produce the respective fine chemical;
b) associate the different sequence and/or expression level of the nucleic acid molecules of the invention with their different capability to produce the respective fine chemical;
c) analyze the sequence and/or the expression of the nucleic acid molecule of the invention in at least a third non-human organism;
d) predict the capability of the at least third organism to produce the respective fine chemical based by comparing the sequence and/or expression of the nucleic acid of the invention to the sequences and/or expression levels of the nucleic acid molecule of the invention of the at least two organisms analyzed in step a.

[0411.1.1.4] to [0430.1.1.4] for the disclosure of these paragraphs see [0411.1.1.1] to [0430.1.1.1] above.

Accordingly the present invention relates to any cell transgenic for any nucleic acid characterized as part of the invention, e.g. conferring the increase of the respective fine chemical threonine in a cell or a non-human organism or a part thereof, e.g. the nucleic acid molecule of the invention, the nucleic acid construct of the invention, the vector of the invention, the expression cassette according to the invention, or a nucleic acid molecule encoding the polypeptide of the invention, e.g. encoding a polypeptide having an activity as the protein as shown in the respective line in Table II, application no. 4, column 3. Due to the above-mentioned activity the respective fine chemical threonine content in a cell or a non-human organism is increased. For example, due to modulation or manipulation, the cellular activity is increased, in a preferred embodiment in organelles such as plastids or mitochondria, e.g. due to an increased expression or specific activity or specific targeting of the subject matters of the invention in a cell or a non-human organism or a part thereof especially in organelles such as plastids or mitochondria, or in another embodiment in the cytosol. Transgenic for a polypeptide having a protein or a protein activity means herein that due to modulation or manipulation of the genome, the activity of protein as shown in the respective line in Table II, application no. 4, column 3 or a protein as shown in the respective line in Table II, application no. 4, column 3-like activity is increased in the cell or non-human organism or part thereof, especially in organelles such as plastids or mitochondria, or especially in the cytosol. Examples are described above in context with the process of the invention.

for the disclosure of this paragraph see [0432.1.1.1] above.

A naturally occurring expression cassette—for example the naturally occurring combination of the promoter of the gene encoding a protein as shown in the respective line in Table II, application no. 4, column 5 or 8, preferably the coding region thereof, or fragments or homologs thereof with the corresponding encoding gene—becomes a transgenic expression cassette when it is modified by non-natural, synthetic “artificial” methods such as, for example, mutagenesis. Such methods have been described (U.S. Pat. No. 5,565,350; WO 00/15815; also see above).

[0434.1.1.4] to [0435.1.1.4] for the disclosure of these paragraph see [0434.1.1.1] to [0435.1.1.1] above.

Transgenic plants comprising the respective fine chemical(s) synthesized in the process according to the invention can be marketed directly without isolation of the fine chemical synthesized. In the process according to the invention, plants are understood as meaning all plant parts, plant organs such as leaf, stalk, root, tubers or seeds or propagation material or harvested material or the intact plant. In this context, the seed encompasses all parts of the seed such as the seed coats, epidermal cells, seed cells, endosperm or embryonic tissue. The fine chemical produced in the process according to the invention may, however, also be isolated from the plant. In case of threonine this can be in free form or bound to proteins. Fine chemical(s) produced by this process can be harvested by harvesting the non-human organisms either from the culture in which they grow or from the field. For example, this can be done via squeezing, grinding and/or extraction, salt precipitation and/or ion-exchange chromatography of the plant parts, preferably the plant seeds, plant fruits, plant tubers and the like.

[0440.1.1.4] to [0440.1.1.4] for the disclosure of these paragraphs see [0437.1.1.1] to [0440.1.1.1] above.

[0442.1.1.4] to [0454.1.1.4] for the disclosure of these paragraphs see [0442.1.1.1] to [0454.1.1.1] above.

In one embodiment, the present invention relates to a method for the identification of a gene product conferring the production of or an increase in the fine chemical production in a non-human cell, comprising the following steps:
(a) contacting, e.g. hybridizing, a, some or all nucleic acid molecules of a sample, e.g. cells, tissues, plants or microorganisms or a nucleic acid library, which can contain a candidate gene encoding a gene product conferring the production of or an increase in the fine chemical after expression, with the nucleic acid molecule of the present invention as shown in the respective line in column 5 or 8 of Table I A or B of application no. 4, preferably the coding region thereof, or a fragment or homolog thereof;
(b) identifying the nucleic acid molecules, which hybridize under relaxed stringent conditions with the nucleic acid molecule of the present invention in particular to the nucleic acid molecule sequence shown in the respective line in Table I, application no. 4, columns 5 or 8, preferably the coding region thereof, of a fragment or a homolog thereof, preferably in Table I B, application no. 4, columns 5 or 8, preferably the coding region thereof, of a fragment or a homolog thereof, and, optionally, isolating the full length cDNA clone or complete genomic clone;
(c) introducing the candidate nucleic acid molecules or a fragment thereof in host cells, preferably in a plant cell or a microorganism, appropriate for producing the fine chemical;
(d) expressing the identified nucleic acid molecules in the host cells for which the production of or the increased production of the fine chemical is desired;
(e) assaying the fine chemical level in the non-human host cells; and
(f) identifying the nucleic acid molecule and/or its gene product which expression confers the production of or an increase in the fine chemical level in the non-human host cell after expression compared to the wild type.

for the disclosure of this paragraph see [0456.1.1.1] above.

In another embodiment, the present invention relates to a method for the identification of a gene product conferring the production of or an increase in the fine chemical production in a cell, comprising the following steps:
(a) identifying a nucleic acid molecule of a non-human organism, which is at least 20%, 25%, 30%, 35%, 40%, 50%, 60%, 70%, 80%, 90%, 95%, 96%, 97%, 98%, 99%, 99.5% or more homolog to the nucleic acid molecule encoding a polypeptide comprising the polypeptide molecule as shown in the respective line in column 5 or 8 of Table II, application no. 4, or comprising a consensus sequence or a polypeptide motif as shown in the respective line in column 8 of Table IV, application no. 4, or being encoded by a nucleic acid molecule comprising a polynucleotide as shown in the respective line in column 5 or 8 of Table I, application no. 4, preferably the coding region thereof, or a homologue thereof as described herein, for example via homology search in a data bank;
(b) introducing the candidate nucleic acid molecule in non-human host cells, preferably in a plant cell or a microorganism, appropriate for producing the fine chemical;
(c) expressing the identified nucleic acid molecule in the non-human host cells;
(d) assaying the fine chemical level in the non-human host cells; and
(e) identifying the nucleic acid molecule and its gene product which expression confers the production of or an increase in the fine chemical level in the non-human host cell after expression compared to the wild type.

for the disclosure of this paragraph see [0458.1.1.1] above

Further, the nucleic acid molecules disclosed herein, in particular the nucleic acid molecules shown in the respective line in column 5 or 8 of Table I A or B of application no. 4, may be sufficiently homologous to the sequences of related species such that these nucleic acid molecules may serve as markers for the construction of a genomic map in related organism or for association mapping. Furthermore natural variation in the genomic regions corresponding to nucleic acids disclosed herein, in particular the nucleic acid molecule shown in the respective line in column 5 or 8 of Table I A or B, of application no. 4 or homologous thereof, may lead to variation in the activity of the proteins disclosed herein, in particular the proteins comprising polypeptides as shown in the respective lines in column 5 or 8 of Table II A or B, of application no. 4 or their homologous, or comprising the consensus sequence or the polypeptide motif as shown in the respective line in column 8 of Table IV, and in consequence in a natural variation of an increased fine chemical production.

In consequence natural variation eventually also exists in form of more active allelic variants leading already to a relative increase in the fine chemical. Different variants of the nucleic acid molecules disclosed herein, in particular the nucleic acid molecules comprising the nucleic acid molecules as shown in the respective line in column 5 or 8 of Table I A or B of application no. 4, preferably the coding region thereof, which corresponds to different levels of increase in fine chemical, e.g. different levels of increase in fine chemical, can be indentified and used for marker assisted breeding for an enhanced production of the fine chemical.

Natural variation may also exist in the regulatory regions, e.g. the promoters of the natural genes comprising the nucleic acid molecules as shown in the respective line column 5 or 8 of Table I A or B of application no. 4, preferably the coding region thereof, which can similarly correspond to different levels of increase in the fine chemical, e.g. different levels of increase in the fine chemical can be identified and used for marker assisted breeding for an enhanced production of the fine chemical.

Accordingly, the present invention relates to a method for breeding plants for the production of the fine chemical, comprising
(a) selecting a first plant variety with an increased production of the fine chemical, e.g. based on an increased expression of a nucleic acid molecule of the invention as disclosed herein, in particular of a nucleic acid molecule comprising a nucleic acid molecule as shown in the respective line in column 5 or 8 of Table I A or B of application no. 4, preferably the coding region thereof, or fragments or homologs thereof, or a polypeptide comprising a polypeptide as shown in the respective line in column 5 or 8 of Table II A or B of application no. 4, or fragments or homologs thereof, or comprising a consensus sequence or a polypeptide motif as shown in the respective line in column 8 of Table IV, or a homolog thereof, as described herein;
(b) associating the level of increased production of the fine chemical, with the expression level or the genomic structure of a gene encoding said polypeptide or said nucleic acid molecule;
(c) crossing the first plant variety with a second plant variety, which significantly differs in its level of fine chemical; and
(d) identifying, which of the offspring varieties has got increased production of the fine chemical by the expression level of said polypeptide or nucleic acid molecule or the genomic structure of the gene(s) encoding said polypeptide or nucleic acid molecule of the invention.

[0462.1.1.4] to [0482.1.1.4] for the disclosure of these paragraphs see [0462.1.1.1] to [0482.1.1.1] above.

In a further embodiment the present invention is characterized by the features as disclosed in the following items:

Item 1. A process for the production of the fine chemical threonine
(a) increasing or generating one or more activities selected from the group consisting of (DL)-glycerol-3-phosphatase, 2,3-dihydroxyphenylpropionate 1,2-dioxygenase, 2-C-methyl-D-erythritol 4-phosphate cytidylyltransferase, 2-oxoglutarate dehydrogenase E1 subunit, 3-phosphoglycerate dehydrogenase, 47266012-protein, 49747384_SOYBEAN-protein, ABC metal ion transporter substrate-binding protein, ABC transporter permease protein, acetolactate synthase small subunit, acetyltransferase, acyl-CoA dehydrogenase, acyl-CoA synthase, argininosuccinate lyase, aspartate aminotransferase, aspartate kinase, At1g09680-protein, At4g32480-protein, At5g16650-protein, ATP synthase subunit beta, ATP-binding component of a transport system, AX653549-protein,b1003-protein, b1108-protein,b1445-protein, b1522-protein,b1670-protein,b1930-protein, b2360-protein, b2399-protein, b2513-protein, b2613-protein, b2739-protein, b2812-protein, b3083-protein, b3442-protein, b3646-protein, b3989-protein, b4029-protein, b4056-protein, b4250-protein, beta-galactosidase, beta-hydroxylase, bifunctional aspartokinase/homoserine dehydrogenase, bifunctional purine biosynthesis protein, branched chain amino acid ABC transporter ATP-binding protein, branched chain aminotransferase, branched-chain amino acid ABC transporter permease protein, calcium-dependent protein kinase, carbamoyl-phosphate synthase subunit, CCAAT-binding transcription factor, CDP-diacylglycerol-serine O-phosphatidyltransferase, cell division control protein, cell division protein, chaperone protein CIpB, coproporphyrinogen III oxidase, cyclin, cyclin D , D-3-phosphoglycerate dehydrogenase, dihydrolipoamide acetyltransferase, dihydroxyacid dehydratase, DNA helicase II, DnaJ-like chaperone, D-serine dehydratase, elongation factor Tu, enoyl-CoA hydratase, enterobacterial common antigen polymerase, eukaryotic translation initiation factor 5, ferredoxin, GDP-mannose dehydrogenase, geranylgeranyl pyrophosphate synthase, gibberellin 20-oxidase, glucose dehydrogenase, glucose-6-phosphate 1-dehydrogenase, glutamate-ammonia-ligase, glutaredoxin, glutathione S-transferase, glycogen (starch) synthase, glycogen synthase, glycoprotease, glycosyl transferase, heat shock protein, heat shock transcription factor, hexose transporter, histone acetyltransferase, homocitrate synthase, homoserine dehydrogenase, hydrolase, isochorismate synthase, ketol-acid reductoisomerase, kinase, L-asparaginase, major facilitator superfamily transporter protein, malate dehydrogenase, malic enzyme, membrane transport protein, metaldependent hydrolase, methyltransferase, mitochondrial dicarboxylate transporter, monothiol glutaredoxin, monthiol glutaredoxin, murein transglycosylase, NADP-dependent malic enzyme, nitrate/nitrite transport protein, oxireductase, peptidase, permease protein of peptide ABC transporter, phosphate transporter, phosphoanhydride phosphorylase, phosphoenolpyruvate carboxykinase, phosphoribosylaminoimidazole carboxylase catalytic subunit, photosystem II protein, polyphosphate kinase, protein kinase, protein phosphatase, pyruvate carboxylase, pyruvate kinase, restriction alleviation protein, sec-independent protein translocase, sensor histidine kinase, serine protease, sll0418-protein, slr0383-protein, succinyl-CoA synthetase beta chain, thioredoxin, threonine synthase, transcription factor, transcriptional regulator, transcriptional regulator protein, transport protein, TTC0881-protein, uroporphyrin-III C-methyltransferase, valine-pyruvate transaminase, XM_{—}473199-protein, ycl027w-protein, ydr183w-protein, YEL030C-A-protein, yfl054c-protein, ygl237c-protein, yhr131c-protein, YJL127W-A-protein, YKL038W-protein, zinc finger protein, and Zm_{—}4842_BE510522-protein, in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of threonine or a composition comprising threonine in said non-human organism or in the culture medium surrounding said non-human organism.

Item 2. A process for the production of a respective fine chemical threonine, which comprises
(A) (i) increasing or generating of the expression of; and/or
(ii) increasing or generating the expression of an expression product of; and/or
(iii) increasing or generating one or more activities of an expression product encoded by;
at least one respective nucleic acid molecule comprising a nucleic acid molecule selected from the group consisting of:
(a) a nucleic acid molecule encoding the polypeptide shown in column 5 or 8 of Table II, preferably Table II B, application no.4, or a homolog or a fragment thereof;
(b) a nucleic acid molecule shown in column 5 or 8 of Table I, preferably Table I B, application no.4, or a homolog or a fragment thereof (preferably the coding region thereof);
(c) a nucleic acid molecule, which, as a result of the degeneracy of the genetic code, can be derived from a polypeptide sequence depicted in column 5 or 8 of Table II, preferably Table II B, application no.4;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 4, or the coding region thereof;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no.4;
(i) a nucleic acid molecule encoding a polypeptide having the activity represented by a respective protein as depicted in column 5 of Table II, application no. 4;
(j) a nucleic acid molecule which comprises a polynucleotide, which is obtained by amplifying a cDNA library or a genomic library using the primers as depicted in column 8 of Table III, application no. 4; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto;
in a non-human organism or a part thereof, preferably a microorganism, a plant cell, a plant or a part thereof, as compared to a corresponding non-transformed wild type non-human organism or a part thereof; and
(B) growing the non-human organism or a part thereof under conditions which permit the production of threonine or a composition comprising threonine in said non-human organism or in the culture medium surrounding said non-human organism.

Item 3. A process of items 1 or 2, comprising of recovering threonine in its free or bound form.

Item 4. The process of any one of items 1 to 3, comprising the following steps:
(a) selecting a non-human organism or a part thereof expressing a polypeptide encoded by the nucleic acid molecule characterized in item 2;
(b) mutagenizing the selected non-human organism or the part thereof;
(c) comparing the activity or the expression level of said polypeptide in the mutagenized non-human organism or the part thereof with the activity or the expression of said polypeptide of the selected non-human organisms or the part thereof;
(d) selecting the mutated non-human organisms or parts thereof, which comprise an increased activity or expression level of said polypeptide compared to the selected non-human organism or the part thereof;
(e) optionally, growing and cultivating the non-human organisms or the parts thereof;
and
(f) recovering, and optionally isolating, the free or bound threonine produced by the selected mutated non-human organisms or parts thereof.

Item 5. The process of any one of items 1 to 4 wherein the activity of said protein or the expression of said nucleic acid molecule is increased or generated transiently or stably.

Item 6. An isolated nucleic acid molecule comprising a nucleic acid molecule selected from the group consisting of:
(a) a nucleic acid molecule encoding a polypeptide shown in Table II, application no. 4, column 5 or 8, preferably shown in Table II B, application no. 4, column 8, or a homolog or a fragment thereof;
(b) a nucleic acid molecule shown in Table I, application no. 4, column 5 or 8, preferably shown in Table I B, application no. 4, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 4, preferably in column 8 of Table II B, application no. application no.;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 3, preferably shown in column 8 of Table I B, application no. 4, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 4;
(i) a nucleic acid molecule encoding a polypeptide having the activity represented by a respective protein as depicted in column 5 of Table II, application no. 4;
(j) a nucleic acid molecule comprising a polynucleotide which is obtained by amplifying a cDNA library or a genomic library using the primers in Table III, application no. 4, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (i);
or a nucleic acid molecule comprising a sequence which is complementary thereto.

Item 7. A nucleic acid construct which confers the expression of the nucleic acid molecule as defined in item 6, comprising one or more regulatory elements.

Item 8. An expression cassette comprising
(a) a promoter, preferably selected from the group consisting of Big35S, PCUbi, Super and USP;
(b) a nucleic acid molecule as defined in item 6.

Item 9. A vector comprising the nucleic acid molecule as defined in item 6 or the nucleic acid construct as claimed in item 7 or the expression cassette as claimed in item 8.

Item 10. A host cell or a plant organelle, which has been transformed with the vector as claimed in item 9 or the nucleic acid molecule as defined in item 6 or the nucleic acid construct as claimed in item 7 or the expression cassette as claimed in item 8.

Item 11. The host cell of item 10, which is a plant cell or a microorganism.

Item 12. A process for producing a polypeptide, wherein the polypeptide is expressed in a host cell as claimed in item 10 or 11.

Item 13. A polypeptide produced by the process as claimed in item 12 or encoded by the nucleic acid molecule as defined in item 6.

Item 14. An antibody, which binds specifically to the polypeptide as claimed in item 13.

Item 15. A plant tissue, propagation material, harvested material or a plant comprising the host cell as claimed in item 10 or 11.

Item 16. A process for the identification of a compound conferring an increase in threonine production in a non-human organism, comprising the steps:
(a) culturing a plant cell or tissue or microorganism or maintaining a plant expressing the polypeptide encoded by the nucleic acid molecule as defined in item 6 conferring an increase in the amount of threonine in a non-human organism or a part thereof and a readout system capable of interacting with the polypeptide under suitable conditions which permit the interaction of the polypeptide with said readout system in the presence of a compound or a sample comprising a plurality of compounds and capable of providing a detectable signal in response to the binding of a compound to said polypeptide under conditions which permit the expression of said readout system and of the polypeptide encoded by the nucleic acid molecule of item 6 conferring an increase in the amount of threonine in a non-human organism or a part thereof;
(b) identifying if the compound is an effective agonist by detecting the presence or absence or increase of a signal produced by said readout system.

Item 17. A method for the production of an agricultural composition comprising the steps of the method of item 16 and formulating the compound identified in item 16 in a form acceptable for an application in agriculture.

Item 18. A composition comprising the nucleic acid molecule as defined in item 6, the polypeptide as claimed in item 13, the nucleic acid construct as claimed in item 7, the expression cassette as claimed in item 8, the vector as claimed in item 9, the compound as claimed in item 16, the antibody as claimed in item 14, and optionally an agricultural acceptable carrier.

Item 19. Use of the nucleic acid molecule as defined in item 6 for the identification of a nucleic acid molecule conferring an increase in threonine after expression.

Item 20. Cosmetic, pharmaceutical, food or feed composition comprising the nucleic acid moleculeas defined in item 6, the polypeptide as claimed in item 13, the nucleic acid construct as claimed in item 7, the expression cassette as claimed in item 8, the vector as claimed in item 9, the antibody as claimed in item 14 , the plant or a part thereof, or plant tissue as claimed in item 15, the harvested material or propagation material as claimed in item 15 or the host cell of item as claimed in items 10 or 11.

Item 21. Use of the nucleic acid molecule as defined in item 6, the polypeptide as claimed in item 13, the nucleic acid construct as claimed in item 7, the expression cassette as claimed in item 8, the vector as claimed in item 9, the plant or plant tissue as claimed in item 15, or the host cell as claimed in item 10 to 11 for the production of plant resistant to a herbicide inhibiting the production of threonine.

[0483.1.1.4] to [0494.1.1.4] for the disclosure of these paragraphs see [0483.1.1.1] to [0494.1.1.1] above.

### Example 11a

### PCR Amplification of the Sequences

Unless otherwise specified, standard methods as described in Sambrook et al., Molecular Cloning: A laboratory manual, Cold Spring Harbor 1989, Cold Spring Harbor Laboratory Press are used.

The inventive sequences as shown in the respective line in Table I, column 5, preferably the coding region thereof, were amplified by PCR as described in the protocol of the Pfu Ultra, Pfu Turbo or Herculase DNA polymerase (Stratagene). The composition for the protocol of the Pfu Ultra, Pfu Turbo or Herculase DNA polymerase was as follows: 1×PCR buffer (Stratagene), 0.2 mM of each dNTP, 100 ng genomic DNA of Saccharomyces cerevisiae (strain S288C; Research Genetics, Inc., now Invitrogen), Escherichia coli (strain MG1655; E. coli Genetic Stock Center), Synechocystis sp. (strain PCC6803), Azotobacter vinelandii (strain N.R. Smith,16), Thermus thermophilus (HB8) or 50 ng cDNA from various tissues and development stages of Arabidopsis thaliana (ecotype Columbia), Physcomitrella patens, Glycine max (variety Resnick), Brassica napus, Oryza sativa or Zea mays (variety B73, Mo17, A188), 50 pmol forward primer, 50 pmol reverse primer, with or without 1 M Betaine, 2.5 u Pfu Ultra, Pfu Turbo or Herculase DNA polymerase.

The amplification cycles were as follows:

1 cycle of 2-3 minutes at 94-95° C., then 25-36 cycles with 30-60 seconds at 94-95° C., 30-45 seconds at 50-60° C. and 210-480 seconds at 72° C., followed by 1 cycle of 5-10 minutes at 72° C., then 4-16° C.—preferably for Saccharomyces cerevisiae, Escherichia coli, Synechocystis sp., Azotobacter vinelandii, Thermus thermophilus.

In case of Arabidopsis thaliana, Brassica napus, Glycine max, Oryza sativa, Physcomitrella patens, Zea mays the amplification cycles were as follows:

1 cycle with 30 seconds at 94° C., 30 seconds at 61° C., 15 minutes at 72° C., then 2 cycles with 30 seconds at 94° C., 30 seconds at 60° C., 15 minutes at 72° C., then 3 cycles with 30 seconds at 94° C., 30 seconds at 59° C., 15 minutes at 72° C., then 4 cycles with 30 seconds at 94° C., 30 seconds at 58° C., 15 minutes at 72° C., then 25 cycles with 30 seconds at 94° C., 30 seconds at 57° C., 15 minutes at 72° C., then 1 cycle with 10 minutes at 72° C., then finally 4-16° C.

RNAs were generated with the RNeasy Plant Kit according to the standard protocol (Qiagen) and Superscript II Reverse Transkriptase was used to produce double stranded cDNA according to the standard protocol (Invitrogen).

ORF specific primer pairs for the genes to be expressed are shown in the respective line in Table III, column 8. The following adapter sequences were added to Saccharomyces cerevisiae ORF specific primers (see Table III) for cloning purposes:

SEQ ID NO: 20

| | | |
|---|---|---|
| i) | foward primer: | 5′-GGAATTCCAGCTGACCACC-3′ |

SEQ ID NO: 21

| | | |
|---|---|---|
| ii) | reverse primer: | 5′-GATCCCCGGGAATTGCCATG-3′ |

These adaptor sequences allow cloning of the ORF into the various vectors containing the Resgen adaptors, see table column E of Table c.

The following adapter sequences were added to Saccharomyces cerevisiae, Escherichia coli, Synechocystis sp., Azotobacter vinelandii, Thermus thermophilus, Arabidopsis thaliana, Brassica napus, Glycine max, Oryza sativa , Physcomitrella patens, or Zea mays ORF specific primers for cloning purposes:

SEQ ID NO: 22
iii) forward primer: 5′-TTGCTCTTCC-3′
SEQ ID NO: 23
iiii) reverse primer: 5′-TTGCTCTTCG-3′

The adaptor sequences allow cloning of the ORF into the various vectors containing the Colic adaptors, see table column E of Table c.

Therefore for amplification and cloning of Saccharomyces cerevisiae SEQ ID NO: 62524, a primer consisting of the adaptor sequence i) and the ORF specific sequence SEQ ID NO: 62712 and a second primer consisting of the adaptor sequence ii) and the ORF specific sequence SEQ ID NO: 62713 were used.

For amplification and cloning of Escherichia coli SEQ ID NO: 6818, a primer consisting of the adaptor sequence iii) and the ORF specific sequence SEQ ID NO: 7076 and a second primer consisting of the adaptor sequence iiii) and the ORF specific sequence SEQ ID NO: 7077 were used.

For amplification and cloning of Synechocystis sp. SEQ ID NO: 11423, a primer consisting of the adaptor sequence iii) and the ORF specific sequence SEQ ID NO: 11465 and a second primer consisting of the adaptor sequence iiii) and the ORF specific sequence SEQ ID NO: 11466 were used.

For amplification and cloning of Arabidopsis thaliana SEQ ID NO: 352, a primer consisting of the adaptor sequence iii) and the ORF specific sequence SEQ ID NO: 380 and a second primer consisting of the adaptor sequence iiii) and the ORF specific sequence SEQ ID NO: 381 were used.

For amplification and cloning of Azotobacter vinelandii SEQ ID NO: 5557, a primer consisting of the adaptor sequence iii) and the ORF specific sequence SEQ ID NO: 6033 and a second primer consisting of the adaptor sequence iiii) and the ORF specific sequence SEQ ID NO: 6034 were used.

For amplification and cloning of Glycine max SEQ ID NO: 69, a primer consisting of the adaptor sequence iii) and the ORF specific sequence SEQ ID NO: 343 and a second primer consisting of the adaptor sequence iiii) and the ORF specific sequence SEQ ID NO: 344 were used.

For amplification and cloning of Oryza sativa SEQ ID NO: 34301, a primer consisting of the adaptor sequence iii) and the ORF specific sequence SEQ ID NO: 34595 and a second primer consisting of the adaptor sequence iiii) and the ORF specific sequence SEQ ID NO: 34596 were used.

For amplification and cloning of Thermus thermophilus SEQ ID NO: 12974, a primer consisting of the adaptor sequence iii) and the ORF specific sequence SEQ ID NO: 13372 and a second primer consisting of the adaptor sequence iiii) and the ORF specific sequence SEQ ID NO: 13373 were used.

For amplification and cloning of Zea mays SEQ ID NO: 68413, a primer consisting of the adaptor sequence iii) and the ORF specific sequence SEQ ID NO: 68531 and a second primer consisting of the adaptor sequence iiii) and the ORF specific sequence SEQ ID NO: 68532 were used.

Following these examples every sequence disclosed in Table I, preferably column 5, especially the coding region thereof can be cloned by fusing the adaptor sequences to the respective specific primers sequences as disclosed in Table III, column 8 using the respective vectors shown in Table c.

[0496.1.1.4] to [0499.1.1.4] for the disclosure of these paragraphs see [0496.1.1.1] to [0499.1.1.1] above.

### Example 11e

### Cloning of Inventive Sequences as Sown in Table I, Column 5 in the Different Expression Vectors

For cloning of for example SEQ ID NO: 6818 from Escherichia coli or any other ORF from Escherichia coli, Synechocystis sp., Azotobacter vinelandii, Thermus thermophilus, Arabidopsis thaliana, Glycine max, Oryza sativa, or Zea mays the vector DNA was treated with the restriction enzymes Pacl and Ncol following the standard protocol (MBI Fermentas).

For cloning for example the ORFs of SEQ ID NO: 62524 from Saccharomyces cerevisiae or any other ORF from Saccharomyces cerevisiae into vectors containing the Resgen adaptor sequence the respective vector DNA was treated with the restriction enzyme Ncol.

For cloning of for example ORFs SEQ ID NO: 14885 from Saccharomyces cerevisiae or any other ORF from Saccharomyces cerevisiae into vectors containing the Colic adaptor sequence, the respective vector DNA was treated with the restriction enzymes Pacl and Ncol following the standard protocol (MBI Fermentas).

In all cases the reaction was stopped by inactivation at 70° C. for 20 minutes and purified over QIAquick or NucleoSpin Extract II columns following the standard protocol (Qiagen or Macherey-Nagel).

Then the PCR-product representing the amplified ORF with the respective adapter sequences and the vector DNA were treated with T4 DNA polymerase according to the standard protocol (MBI Fermentas) to produce single stranded overhangs with the parameters 1 unit T4 DNA polymerase at 37° C. for 2-10 minutes for the vector and 1-2 u T4 DNA polymerase at 15-17° C. for 10-60 minutes for the PCR product representing SEQ ID NO: 6818.

The reaction was stopped by addition of high-salt buffer and purified over QIAquick or NucleoSpin Extract II columns following the standard protocol (Qiagen or Macherey-Nagel).

According to this example the skilled person is able to clone all sequences disclosed in Table I, preferably column 5 or column 8, especially the coding region thereof.

Approximately 30-60 ng of prepared vector and a defined amount of prepared amplificate were mixed and hybridized at 65° C. for 15 minutes followed by 37° C. 0,1 ° C./1 seconds, followed by 37° C. 10 minutes, followed by 0,1 ° C./1 seconds, then 4-10 ° C.

The ligated constructs were transformed in the same reaction vessel by addition of competent E. coli cells (strain DHSalpha) and incubation for 20 minutes at 1° C. followed by a heat shock for 90 seconds at 42° C. and cooling to 1-4° C. Then, complete medium (SOC) was added and the mixture was incubated for 45 minutes at 37° C. The entire mixture was subsequently plated onto an agar plate with 0.05 mg/ml kanamycin and incubated overnight at 37° C.

The outcome of the cloning step was verified by amplification with the aid of primers which bind upstream and downstream of the integration site, thus allowing the amplification of the insertion. The amplifications were carried out as described in the protocol of Taq DNA polymerase (Gibco-BRL).

The amplification cycles were as follows:

1 cycle of 1-5 minutes at 94° C., followed by 35 cycles of in each case 15-60 seconds at 94° C., 15-60 seconds at 50-66° C. and 5-15 minutes at 72° C., followed by 1 cycle of 10 minutes at 72° C., then 4-16° C.

Several colonies were checked, but only one colony for which a PCR product of the expected size was detected was used in the following steps.

A portion of this positive colony was transferred into a reaction vessel filled with complete medium (LB) supplemented with kanamycin and incubated overnight at 37° C.

The plasmid preparation was carried out as specified in the Qiaprep or NucleoSpin Multi-96 Plus standard protocol (Qiagen or Macherey-Nagel).

[0501.1.1.4] to [0503.1.1.4] for the disclosure of these paragraphs see [0501.1.1.1] to [0503.1.1.1] above. Table d showing results of plant analyses

TABLE d

| SeqID | Target | Locus | Metabolite | Source | Promotor | Method | Min | Max |
|---|---|---|---|---|---|---|---|---|
| 15187 | non- | 47266012_ | threonine | ARA_LEAF | p-PcUBI | GC | 19 | 26 |
| | targeted | SOYBEAN | | | | | | |
| 69 | non- | 49747384_ | threonine | ARA_LEAF | p-PcUBI | LC | 31 | 100 |
| | targeted | SOYBEAN | | | | | | |
| 16883 | plastidic | 59582753_ | threonine | ARA_LEAF | p-PcUBI | GC | 16 | 54 |
| | | SOYBEAN | | | | | | |
| 352 | non- | At1g09680 | threonine | ARA_LEAF | p-PcUBI | GC | 18 | 69 |
| | targeted | | | | | | | |
| 627 | non- | At1g31230 | threonine | ARA_LEAF | Big35S | LC | 231 | 2283 |
| | targeted | | | | | | | |
| 17968 | non- | At1g36730 | threonine | ARA_LEAF | p-PcUBI | GC | 18 | 84 |
| | targeted | | | | | | | |
| 18070 | non- | At1g43850 | threonine | ARA_LEAF | p-PcUBI | GC | 18 | 144 |
| | targeted | | | | | | | |
| 812 | non- | At1g48040 | threonine | ARA_LEAF | p-PcUBI | LC | 24 | 88 |
| | targeted | | | | | | | |
| 18235 | non- | At1g48260 | threonine | ARA_LEAF | p-PcUBI | LC | 29 | 127 |
| | targeted | | | | | | | |
| 19419 | non- | At1g72770 | threonine | ARA_LEAF | p-PcUBI | LC | 41 | 302 |
| | targeted | | | | | | | |
| 1298 | non- | At2g25070 | threonine | ARA_LEAF | p-PcUBI | LC | 27 | 148 |
| | targeted | | | | | | | |
| 20346 | non- | At2g30540 | threonine | ARA_LEAF | p-PcUBI | GC | 21 | 74 |
| | targeted | | | | | | | |
| 22921 | non- | At3g20910 | threonine | ARA_LEAF | p-PcUBI | GC | 19 | 52 |
| | targeted | | | | | | | |
| 1815 | non- | At3g23000 | threonine | ARA_LEAF | p-PcUBI | GC | 16 | 83 |
| | targeted | | | | | | | |
| 68777 | non- | At3g27540 | threonine | ARA_LEAF | p-PcUBI | GC | 20 | 48 |
| | targeted | | | | | | | |
| 2573 | non- | At3g62930 | threonine | ARA_LEAF | p-PcUBI | LC | 28 | 49 |
| | targeted | | | | | | | |
| 2935 | non- | At3g62950 | threonine | ARA_LEAF | p-PcUBI | LC | 32 | 130 |
| | targeted | | | | | | | |
| 23482 | non- | At4g15660 | threonine | ARA_LEAF | p-PcUBI | LC | 31 | 672 |
| | targeted | | | | | | | |
| 3279 | non- | At4g15670 | threonine | ARA_LEAF | p-PcUBI | LC | 29 | 92 |
| | targeted | | | | | | | |
| 23844 | non- | At4g15690 | threonine | ARA_LEAF | p-PcUBI | LC | 26 | 59 |
| | targeted | | | | | | | |
| 24232 | non- | At4g18880 | threonine | ARA_LEAF | p-PcUBI | LC | 32 | 75 |
| | targeted | | | | | | | |
| 4040 | non- | At4g32480 | threonine | ARA_LEAF | p-PcUBI | GC | 32 | 265 |
| | targeted | | | | | | | |
| 24311 | non- | At4g34160 | threonine | ARA_LEAF | p-PcUBI | GC | 40 | 97 |
| | targeted | | | | | | | |
| 4348 | non- | At4g35310 | threonine | ARA_LEAF | p-PcUBI | LC | 29 | 102 |
| | targeted | | | | | | | |
| 24492 | non- | At5g07200 | threonine | ARA_LEAF | p-PcUBI | GC | 18 | 54 |
| | targeted | | | | | | | |
| 93451 | non- | At5g13280 | threonine | ARA_LEAF | p-PcUBI | GC | 38 | 356 |
| | targeted | | | | | | | |
| 25283 | non- | At5g16650 | threonine | ARA_LEAF | p-PcUBI | GC | 16 | 48 |
| | targeted | | | | | | | |
| 69574 | non- | At5g39950 | threonine | ARA_LEAF | p-PcUBI | GC | 16 | 23 |
| | targeted | | | | | | | |
| 5318 | non- | At5g57050 | threonine | ARA_LEAF | p-PcUBI | GC | 18 | 60 |
| | targeted | | | | | | | |
| 5493 | non- | At5g64920 | threonine | ARA_LEAF | p-PcUBI | LC | 30 | 578 |
| | targeted | | | | | | | |
| 70583 | non- | Avin- | threonine | ARA_LEAF | p-PcUBI | GC | 17 | 41 |
| | targeted | DRAFT_0035 | | | | | | |
| 5557 | non- | Avin- | threonine | ARA_LEAF | p-PcUBI | GC | 33 | 157 |
| | targeted | DRAFT_1495 | | | | | | |
| 26196 | non- | Avin- | threonine | ARA_LEAF | p-PcUBI | GC | 18 | 55 |
| | targeted | DRAFT_1624 | | | | | | |
| 6040 | non- | Avin- | threonine | ARA_LEAF | p-PcUBI | GC | 19 | 37 |
| | targeted | DRAFT_2091 | | | | | | |
| 27021 | non- | Avin- | threonine | ARA_LEAF | p-PcUBI | GC | 25 | 121 |
| | targeted | DRAFT_2344 | | | | | | |
| 27882 | non- | Avin- | threonine | ARA_LEAF | p-PcUBI | GC | 33 | 116 |
| | targeted | DRAFT_2521 | | | | | | |
| 28040 | non- | Avin- | threonine | ARA_LEAF | p-PcUBI | GC | 17 | 61 |
| | targeted | DRAFT_2754 | | | | | | |
| 6075 | non- | Avin- | threonine | ARA_LEAF | p-PcUBI | LC | 29 | 249 |
| | targeted | DRAFT_3028 | | | | | | |
| 93532 | non- | Avin- | threonine | ARA_LEAF | p-PcUBI | GC | 24 | 34 |
| | targeted | DRAFT_3086 | | | | | | |
| 72346 | non- | Avin- | threonine | ARA_LEAF | p-PcUBI | GC | 21 | 25 |
| | targeted | DRAFT_3135 | | | | | | |
| 28738 | non- | Avin- | threonine | ARA_LEAF | p-PcUBI | GC | 20 | 139 |
| | targeted | DRAFT_3159 | | | | | | |
| 29286 | non- | Avin- | threonine | ARA_LEAF | p-PcUBI | GC | 18 | 42 |
| | targeted | DRAFT_3209 | | | | | | |
| 6268 | non- | Avin- | threonine | ARA_LEAF | p-PcUBI | LC | 41 | 1222 |
| | targeted | DRAFT_3546 | | | | | | |
| 6510 | non- | Avin- | threonine | ARA_LEAF | p-PcUBI | GC | 22 | 170 |
| | targeted | DRAFT_5103 | | | | | | |
| 32037 | non- | Avin- | threonine | ARA_LEAF | p-PcUBI | GC | 24 | 136 |
| | targeted | DRAFT_5246 | | | | | | |
| 74662 | non- | Avin- | threonine | ARA_LEAF | p-PcUBI | GC | 17 | 35 |
| | targeted | DRAFT_5579 | | | | | | |
| 33596 | non- | Avin- | threonine | ARA_LEAF | p-PcUBI | GC | 18 | 56 |
| | targeted | DRAFT_6093 | | | | | | |
| 34204 | non- | Avin- | threonine | ARA_LEAF | p-PcUBI | GC | 47 | 68 |
| | targeted | DRAFT_6864 | | | | | | |
| 34301 | non- | AX653549 | threonine | ARA_LEAF | p-PcUBI | GC | 17 | 50 |
| | targeted | | | | | | | |
| 34889 | plastidic | B0004 | threonine | ARA_SEED_2 | p-USP | GC | 23 | 123 |
| 6818 | non- | B0078 | threonine | ARA_SEED_2 | p-USP | GC | 33 | 83 |
| | targeted | | | | | | | |
| 35366 | plastidic | B0115 | threonine | ARA_SEED_2 | p-USP | LC | 35 | 50 |
| 35482 | non- | B0124 | threonine | ARA_LEAF | p-Super | GC | 20 | 127 |
| | targeted | | | | | | | |
| 7081 | non- | B0161 | threonine | ARA_LEAF | p-Super | LC | 36 | 144 |
| | targeted | | | | | | | |
| 35590 | non- | B0221 | threonine | ARA_LEAF | p-Super | GC | 18 | 53 |
| | targeted | | | | | | | |
| 35733 | plastidic | B0344 | threonine | ARA_SEED_2 | p-USP | GC | 36 | 41 |
| 7269 | plastidic | B0348 | threonine | ARA_SEED_2 | p-USP | GC | 36 | 157 |
| 7333 | non- | B0449 | threonine | ARA_LEAF | p-Super | GC | 33 | 123 |
| | targeted | | | | | | | |
| 7686 | non- | B0486 | threonine | ARA_LEAF | p-Super | GC | 29 | 45 |
| | targeted | | | | | | | |
| 35967 | plastidic | B0593 | threonine | ARA_LEAF | p-Super | LC | 31 | 76 |
| 93678 | non- | B0695 | threonine | ARA_LEAF | p-Super | LC | 27 | 50 |
| | targeted | | | | | | | |
| 93793 | plastidic | B0728 | threonine | ARA_LEAF | p-Super | GC | 20 | 50 |
| 7917 | non- | B0898 | threonine | ARA_LEAF | p-Super | GC | 19 | 355 |
| | targeted | | | | | | | |
| 36809 | non- | B0980 | threonine | ARA_SEED_2 | p-USP | GC | 27 | 117 |
| | targeted | | | | | | | |
| 7941 | non- | B1003 | threonine | ARA_LEAF | p-Super | GC | 60 | 144 |
| | targeted | | | | | | | |
| 36937 | non- | B1108 | threonine | ARA_LEAF | p-Super | GC | 17 | 59 |
| | targeted | | | | | | | |
| 77768 | non- | B1348 | threonine | ARA_LEAF | p-Super | LC | 32 | 412 |
| | targeted | | | | | | | |
| 38289 | non- | B1445 | threonine | ARA_LEAF | p-Super | LC | 34 | 72 |
| | targeted | | | | | | | |
| 77774 | non- | B1479 | threonine | ARA_SEED_2 | p-USP | GC | 71 | 160 |
| | targeted | | | | | | | |
| 7947 | non- | B1522 | threonine | ARA_LEAF | p-Super | LC | 30 | 93 |
| | targeted | | | | | | | |
| 78753 | non- | B1670 | threonine | ARA_LEAF | p-Super | GC | 18 | 47 |
| | targeted | | | | | | | |
| 8363 | plastidic | B1854 | threonine | ARA_SEED_2 | p-USP | GC | 28 | 121 |
| 79181 | non- | B1930 | threonine | ARA_LEAF | p-Super | GC | 16 | 77 |
| | targeted | | | | | | | |
| 38947 | non- | B2063 | threonine | ARA_LEAF | p-Super | GC | 17 | 157 |
| | targeted | | | | | | | |
| 39219 | non- | B2360 | threonine | ARA_LEAF | p-Super | GC | 21 | 27 |
| | targeted | | | | | | | |
| 79342 | plastidic | B2366 | threonine | ARA_LEAF | p-Super | GC | 19 | 81 |
| 39237 | non- | B2399 | threonine | ARA_LEAF | p-Super | LC | 29 | 74 |
| | targeted | | | | | | | |
| 39255 | non- | B2405 | threonine | ARA_LEAF | p-Super | GC | 20 | 28 |
| | targeted | | | | | | | |
| 9167 | non- | B2513 | threonine | ARA_LEAF | p-Super | LC | 38 | 74 |
| | targeted | | | | | | | |
| 40665 | non- | B2613 | threonine | ARA_LEAF | p-Super | GC | 19 | 54 |
| | targeted | | | | | | | |
| 80756 | non- | B2701 | threonine | ARA_LEAF | p-Super | GC | 30 | 59 |
| | targeted | | | | | | | |
| 40741 | non- | B2714 | threonine | ARA_LEAF | p-Super | GC | 17 | 68 |
| | targeted | | | | | | | |
| 80906 | non- | B2739 | threonine | ARA_LEAF | p-Super | LC | 28 | 74 |
| | targeted | | | | | | | |
| 81075 | non- | B2747 | threonine | ARA_LEAF | p-Super | GC | 29 | 59 |
| | targeted | | | | | | | |
| 40795 | plastidic | B2812 | threonine | ARA_LEAF | p-Super | GC | 23 | 44 |
| 40795 | non- | B2812 | threonine | ARA_LEAF | p-Super | LC | 28 | 59 |
| | targeted | | | | | | | |
| 81988 | plastidic | B2913 | threonine | ARA_SEED_2 | p-USP | GC | 26 | 200 |
| 41499 | non- | B2957 | threonine | ARA_LEAF | p-Super | GC | 18 | 72 |
| | targeted | | | | | | | |
| 42046 | non- | B3064 | threonine | ARA_SEED_2 | p-Super | GC | 23 | 76 |
| | targeted | | | | | | | |
| 82424 | non- | B3083 | threonine | ARA_LEAF | p-Super | LC | 26 | 46 |
| | targeted | | | | | | | |
| 42502 | non- | B3262 | threonine | ARA_LEAF | p-Super | LC | 28 | 141 |
| | targeted | | | | | | | |
| 94051 | mitochon- | B3429 | threonine | ARA_LEAF | p-Super | GC | 27 | 60 |
| | drial | | | | | | | |
| 94380 | non- | B3442 | threonine | ARA_LEAF | p-Super | LC | 33 | 44 |
| | targeted | | | | | | | |
| 10172 | plastidic | B3572 | threonine | ARA_SEED_2 | p-USP | GC | 24 | 80 |
| 83429 | non- | B3646 | threonine | ARA_LEAF | p-Super | GC | 24 | 35 |
| | targeted | | | | | | | |
| 43248 | non- | B3732 | threonine | ARA_LEAF | p-Super | GC | 17 | 37 |
| | targeted | | | | | | | |
| 10252 | non- | B3771 | threonine | ARA_SEED_2 | p-USP | GC | 26 | 72 |
| | targeted | | | | | | | |
| 94386 | non- | B3774 | threonine | ARA_SEED_2 | p-USP | LC | 29 | 91 |
| | targeted | | | | | | | |
| 43800 | non- | B3793 | threonine | ARA_LEAF | p-Super | GC | 17 | 70 |
| | targeted | | | | | | | |
| 43839 | non- | B3813 | threonine | ARA_SEED_2 | p-USP | GC | 25 | 94 |
| | targeted | | | | | | | |
| 83821 | non- | B3872 | threonine | ARA_LEAF | p-Super | GC | 17 | 51 |
| | targeted | | | | | | | |
| 44372 | non- | B3989 | threonine | ARA_LEAF | p-Super | GC | 22 | 68 |
| | targeted | | | | | | | |
| 83835 | plastidic | B4024 | threonine | ARA_SEED_2 | p-USP | GC | 64 | 1969 |
| 10740 | non- | B4029 | threonine | ARA_LEAF | p-Super | GC | 21 | 213 |
| | targeted | | | | | | | |
| 94542 | non- | B4056 | threonine | ARA_LEAF | p-Super | GC | 18 | 43 |
| | targeted | | | | | | | |
| 94735 | non- | B4250 | threonine | ARA_LEAF | p-Super | LC | 25 | 60 |
| | targeted | | | | | | | |
| 45321 | non- | B4256 | threonine | ARA_LEAF | p-Super | GC | 20 | 95 |
| | targeted | | | | | | | |
| 10811 | non- | GM02LC12622 | threonine | ARA_LEAF | p-PcUBI | GC | 20 | 83 |
| | targeted | | | | | | | |
| 84198 | non- | GM02LC21368 | threonine | ARA_LEAF | p-PcUBI | GC | 30 | 112 |
| | targeted | | | | | | | |
| 46850 | non- | GM02LC44512 | threonine | ARA_LEAF | p-PcUBI | GC | 24 | 60 |
| | targeted | | | | | | | |
| 48138 | non- | Sll0170 | threonine | ARA_LEAF | p-PcUBI | GC | 23 | 96 |
| | targeted | | | | | | | |
| 49828 | non- | Sll0290 | threonine | ARA_LEAF | p-PcUBI | GC | 17 | 53 |
| | targeted | | | | | | | |
| 94741 | plastidic | Sll0374 | threonine | ARA_LEAF | p-PcUBI | GC | 21 | 30 |
| 84869 | plastidic | Sll0378 | threonine | ARA_LEAF | p-PcUBI | GC | 19 | 83 |
| 94942 | non- | Sll0418 | threonine | ARA_LEAF | p-PcUBI | GC | 17 | 30 |
| | targeted | | | | | | | |
| 51198 | non- | Sll0816 | threonine | ARA_LEAF | p-PcUBI | GC | 19 | 91 |
| | targeted | | | | | | | |
| 51268 | plastidic | Sll0891 | threonine | ARA_LEAF | p-PcUBI | GC | 23 | 61 |
| 51632 | mitochon- | Sll0901 | threonine | ARA_LEAF | p-PcUBI | GC | 39 | 78 |
| | drial | | | | | | | |
| 52364 | non- | Sll0945 | threonine | ARA_LEAF | p-PcUBI | GC | 17 | 56 |
| | targeted | | | | | | | |
| 53456 | non- | Sll1393 | threonine | ARA_LEAF | p-PcUBI | GC | 18 | 84 |
| | targeted | | | | | | | |
| 54337 | mitochon- | Sll1450 | threonine | ARA_LEAF | p-PcUBI | GC | 17 | 40 |
| | drial | | | | | | | |
| 95047 | plastidic | Sll1498 | threonine | ARA_LEAF | p-PcUBI | GC | 18 | 30 |
| 11423 | mitochon- | Sll1545 | threonine | ARA_LEAF | p-PcUBI | GC | 17 | 42 |
| | drial | | | | | | | |
| 85743 | mitochon- | Sll1598 | threonine | ARA_LEAF | p-PcUBI | GC | 21 | 35 |
| | drial | | | | | | | |
| 11471 | mitochon- | Sll1917 | threonine | ARA_LEAF | p-PcUBI | GC | 18 | 67 |
| | drial | | | | | | | |
| 95527 | mitochon- | Slr0150 | threonine | ARA_LEAF | p-PcUBI | GC | 18 | 53 |
| | drial | | | | | | | |
| 95744 | non- | Slr0383 | threonine | ARA_LEAF | p-PcUBI | GC | 18 | 35 |
| | targeted | | | | | | | |
| 56153 | plastidic | Slr0597 | threonine | ARA_LEAF | p-PcUBI | GC | 32 | 35 |
| 95857 | mitochon- | Slr0657 | threonine | ARA_LEAF | p-PcUBI | GC | 20 | 46 |
| | drial | | | | | | | |
| 57235 | plastidic | Slr0739 | threonine | ARA_LEAF | p-PcUBI | GC | 16 | 79 |
| 87610 | mitochon- | Slr0862 | threonine | ARA_LEAF | p-PcUBI | LC | 39 | 45 |
| | drial | | | | | | | |
| 87853 | mitochon- | Slr1133 | threonine | ARA_LEAF | p-PcUBI | GC | 23 | 34 |
| | drial | | | | | | | |
| 58731 | plastidic | Slr1739 | threonine | ARA_LEAF | p-PcUBI | LC | 27 | 512 |
| 60301 | non- | TTC0035 | threonine | ARA_LEAF | p-PcUBI | LC | 32 | 102 |
| | targeted | | | | | | | |
| 60859 | non- | TTC0216 | threonine | ARA_LEAF | p-PcUBI | GC | 18 | 35 |
| | targeted | | | | | | | |
| 88875 | non- | TTC0881 | threonine | ARA_LEAF | p-PcUBI | GC | 19 | 34 |
| | targeted | | | | | | | |
| 61553 | non- | TTC0917 | threonine | ARA_LEAF | p-PcUBI | GC | 18 | 26 |
| | targeted | | | | | | | |
| 12974 | non- | TTC1550 | threonine | ARA_LEAF | p-PcUBI | GC | 32 | 94 |
| | targeted | | | | | | | |
| 62244 | non- | XM_473199 | threonine | ARA_LEAF | p-PcUBI | LC | 25 | 82 |
| | targeted | | | | | | | |
| 96022 | plastidic | Yal038w | threonine | ARA_SEED_2 | p-Super | GC | 23 | 101 |
| 62524 | non- | Ybl021c | threonine | ARA_LEAF | Big35S | GC | 17 | 83 |
| | targeted | | | | | | | |
| 62717 | non- | Ybr160w | threonine | ARA_LEAF | Big35S | LC | 25 | 60 |
| | targeted | | | | | | | |
| 96779 | plastidic | Ybr218c | threonine | ARA_LEAF | p-Super | GC | 17 | 27 |
| 96980 | plastidic | Ycl027w | threonine | ARA_LEAF | p-Super | GC | 19 | 26 |
| 63334 | plastidic | Ydr044w | threonine | ARA_LEAF | p-PcUBI | LC | 74 | 194 |
| 90103 | plastidic | Ydr183w | threonine | ARA_LEAF | p-Super | GC | 17 | 53 |
| 96986 | plastidic | Ydr342c | threonine | ARA_LEAF | p-PcUBI | GC | 19 | 92 |
| 97294 | non- | Yel030c-a | threonine | ARA_LEAF | Big35S | GC | 16 | 31 |
| | targeted | | | | | | | |
| 97298 | plastidic | Yel069c | threonine | ARA_LEAF | p-PcUBI | GC | 21 | 35 |
| 64148 | plastidic | Yfl054c | threonine | ARA_LEAF | p-Super | GC | 16 | 23 |
| 64177 | non- | Ygl237c | threonine | ARA_LEAF | Big35S | LC | 27 | 84 |
| | targeted | | | | | | | |
| 97620 | plastidic | Yhr131c | threonine | ARA_LEAF | p-Super | GC | 31 | 32 |
| 14769 | plastidic | Yil053w | threonine | ARA_LEAF | p-Super | GC | 19 | 46 |
| 64563 | plastidic | Yil074c | threonine | ARA_LEAF | p-Super | GC | 22 | 34 |
| 90306 | plastidic | Yir038c | threonine | ARA_LEAF | p-Super | GC | 16 | 25 |
| 64964 | plastidic | Yjl073w | threonine | ARA_LEAF | p-Super | GC | 24 | 27 |
| 97631 | non- | Yjl127w-a | threonine | ARA_LEAF | Big35S | GC | 18 | 61 |
| | targeted | | | | | | | |
| 97635 | non- | Yjl219w | threonine | ARA_SEED_2 | p-PcUBI | GC | 27 | 157 |
| | targeted | | | | | | | |
| 14885 | plastidic | Yjr139c | threonine | ARA_LEAF | p-PcUBI | GC | 16 | 923 |
| 90409 | plastidic | Yjr148w | threonine | ARA_LEAF | p-Super | GC | 18 | 59 |
| 90602 | plastidic | YKL038W | threonine | ARA_LEAF | p-PcUBI | GC | 16 | 45 |
| 98075 | plastidic | Ykr097w | threonine | ARA_LEAF | p-Super | GC | 17 | 35 |
| 90615 | non- | Ylr027c | threonine | ARA_LEAF | Big35S | LC | 57 | 111 |
| | targeted | | | | | | | |
| 98243 | non- | Ylr348c | threonine | ARA_LEAF | p-PcUBI | GC | 23 | 68 |
| | targeted | | | | | | | |
| 91785 | plastidic | Yml123c | threonine | ARA_LEAF | p-Super | GC | 22 | 60 |
| 67684 | plastidic | Yol045w | threonine | ARA_LEAF | p-Super | LC | 32 | 56 |
| 92540 | plastidic | Yor244w | threonine | ARA_LEAF | p-Super | GC | 16 | 24 |
| 98445 | non- | Ypr119w | threonine | ARA_LEAF | Big35S | GC | 16 | 37 |
| | targeted | | | | | | | |
| 68413 | non- | Zm_4842_B | threonine | ARA_LEAF | p-PcUBI | GC | 16 | 78 |
| | targeted | E510522 | | | | | | |

Column 1 shows the SEQ ID NO, Column 2 shows the expression type (targeted or non-targeted), Column 3 shows the “gene name” (locus), Column 4 shows the metabolite analyzed, Column 5 indicates the A. thaliana source tissue analyzed, Column 6 indicates the used promoter for expression, Column 7 indicates the analytical method. Columns 8 and 9 show the minumum and the maximum increase of the analyzed metabolite(in percent) in comparison to the wild type (=ratio_by_weight, given as percent increase).

When the analyses were repeated independently, all results proved to be significant.

[0505.1.1.4] to [0515.1.1.4] for the disclosure of this paragraph see [0505.1.1.1] to [0515.1.1.1] above.

In a further embodiment, the present invention relates to a further process for the production of the fine chemical trypthophane as defined below and corresponding embodiments as described herein as follows.

[0001.1.1.5] to [0008.1.1.5] for the disclosure of these paragraphs see [0001.1.1.1] to [0008.1.1.1] above.

As described above, the essential amino acids are necessary for humans and many mammals, for example for livestock. Tryptophane (L-tryptophane) is one of the most reactive amino acids. At pH 4.0-6.0 tryptophane amino group reacts with aldehydes producing Schiff-bases. On the other hand if the amino group is blocked by acetylation, tryptophane reacts with aldehydes yielding carboline derivatives (1,2,3,4-tetrahydro-carboline-3-carboxylic acid). Tryptophane plays a unique role in defense against infection because of its relative scarcity compared to other amino acids. During infection, the body induces tryptophane-catabolizing enzymes which increase tryptophane's scarcity in an attempt to starve the infecting organisms (Brown R. R., Ozaki Y., Datta S. P. et al., “Implications of interferon-induced tryptophane catabolism in cancer, auto-immune diseases and AIDS” in “Kynurenine and Serotonin Pathways, Schwarcz R. et al. (Eds.), Plenum Press, New York, 1991). In most proteins, tryptophane is the least abundant essential amino acid, comprising approximately 1% of plant proteins and 1.5% of animal proteins. Although the minimum daily requirement for tryptophane is 160 mg for women and 250 mg for men, 500-700 mg are recommended to ensure high-quality protein intake. Actual tryptophane utilization is substantially higher. Men use approximately 3.5 grams of tryptophane to make one days's worth of protein (Peters J. C., “Tryptophane Nutrition and Metabolism: an Overview” in “Kynurenine and Serotonin Pathways”, Schwarcz R. et al., (Eds.), Plenum Press, New York, 1991). The balance is obtained by hepatic recycling of tryptophane from used (catabolized) proteins.

Dietary tryptophane is well absorbed intestinally. About 10% of the tryptophane circulating in the bloodstream is free, and 90% is bound to the protein albumin. The tryptophane binding site on albumin also has affinity for free fatty acids (FFAs), so tryptophane is displaced when FFAs rise, as when fasting.

Although tryptophane is not usually the limiting amino acid in protein synthesis, tryptophane may become insufficient for the normal functioning of other tryptophane-dependent pathways. Numerous lines of research point to tryptophane's central role in regulation of feeding and other behaviors. Tryptophane is not only typically the least abundant amino acid in the liver's free amino acid pool, but liver tryptophane-tRNA levels fall faster during food deprivation than other indispensable amino acids (Rogers Q. R., “The nutritional and metabolic effects of amino acid imbalances” in “Protein Metabolism and Nutrition” Cole D. J. A. (Ed.), Butterworths, London, 1976). Under fasting conditions, and possibly in wasting syndromes, tryptophane may become the rate-limiting amino acid for protein synthesis (Peters J. C., “Tryptophane Nutrition and Metabolism: an Overview” in “Kynurenine and Serotonin Pathways”, Schwarcz R. et al., (Eds.), Plenum Press, New York, 1991).

[0010.1.1.5] to [0011.1.1.5] for the disclosure of these paragraphs see [0010.1.1.1] to [0011.1.1.1] above.

It is an object of the present invention to develop an inexpensive process for the synthesis of tryptophan, especially L-tryptophan. L-tryptophan is one of the amino acids which is limiting.

for the disclosure of this paragraph see [0013.1.1.1] above

Accordingly, in a first embodiment, the invention relates to a process for the production of tryptophan, or, in other words, of the “fine chemical” or “fine chemical of the invention”.

The terms “fine chemical of the invention”, “fine chemical” or “the fine chemical” are used herein equally and relate in context of the paragraphs or sections [0014.1.5.5] to [0514.1.1.5] essentially to the metabolite or the metabolites indicated in column 7, application no. 5 of Tables I, II and IV in the respective line.

Further, the term “in context of any of the paragraphs [0014.1.5.5] to [0514.1.1.5]” as used herein means that for any of said paragraphs [0014.1.5.5] to [0514.1.1.5] the term “the fine chemical” is understood to follow the definition of paragraphs or sections [0014.1.1.5] and [0015.1.5.5], independently whether it refers to any other paragraph or not and whether the reference recites the term “fine chemical” in an other context.

Thus, in cases where one or more paragraphs or sections are incorporated by reference into any of the present paragraphs [0014.1.5.5] to [0514.1.1.5], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to the definition of paragraph [0014.1.5.5] and [0015.1.5.5].

Accordingly, in one embodiment, the term “fine chemical of the invention”, “fine chemical” or “the fine chemical” as used herein (alone or in combination, like in FCRP) means “tryptophan in context of the nucleic acid or polypeptide sequences listed in the respective same line of any one of Tables I to IV of application no. 5 and indicating in column 7 the metabolite “tryptophan”.

In one embodiment, the term tryptophan or the term “fine chemical” mean in context of the paragraphs or sections [0014.1.5.5] to [0514.1.1.5] at least one chemical compound with an activity of the above mentioned tryptophan, respectively.

Accordingly, in one embodiment, the terms “fine chemical of the invention”, “fine chemical” or “the fine chemical” mean in context of any of the paragraphs [0014.1.1.5] to [0514.1.1.5] tryptophan, preferably the L-enantiomer of tryptophan, its salts, ester or amides in free form or bound form. In a preferred embodiment, the term “the fine chemical” means tryptophan or its salts, in free form or bound to proteins. In a preferred embodiment, the term “the fine chemical” means the L-enantiomer of tryptophan.

On the other hand in case “tryptophan” is stated it means tryptophan itself, its salts, ester or amides in free form or bound form, preferably the L-enantiomer of tryptophan, its salts, ester or amides in free form or bound to proteins. In a preferred embodiment “tryptophan” means the Lenantiomer of tryptophan in free form. In another preferred embodiment “tryptophan” means the L-enantiomer of tryptophan bound form.

Further, the term “in context of any of the paragraphs [0014.1.5.5] to [0514.1.1.5]” as used herein means that for any of said paragraphs [0014.1.5.5] to [0514.1.1.5] the term “the fine chemical” is understood to follow the definition of section [0014.1.5.5] or section [0015.1.1.5], independently whether it refers to any other paragraph or not and whether the reference recites the term “fine chemical” in an other context.

Thus, in cases where one or more paragraphs or sections are incorporated by reference into any of the present paragraphs [0014.1.5.5] to [0514.1.1.5], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to this definition of this paragraph [0015.1.5.5].

Further, the term “fine chemicals” as used herein relates to compositions comprising said fine chemical(s), i.e. comprising tryptophan, respectively.

Accordingly, the present invention relates to a process for the production of tryptophan, which comprises
(a) increasing or generating one or more activities selected from the group consisting of glucose-6-phosphate 1-dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of At1g19800-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of cullin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of eukaryotic translation initiation factor 5 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of CBL-interacting protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of At2g33510-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of harpin-induced family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of DNA mismatch repair protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of eukaryotic translation initiation factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of glycosyl transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of auxin response factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of monthiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of At4g32480-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of gibberellin 20-oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of At5g16650-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of at5g21910-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of RNA-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of malic enzyme in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of elongation factor Tu in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of enoyl-CoA hydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of enoyl-CoA hydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of glutamate-ammonia-ligase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of Chaperone protein CIpB in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of electron transfer flavoprotein subunit beta in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of threonine synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of transaldolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of L-ribulose-5-phosphate 4-epimerase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of glucose dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of ATP-binding component of a transport system in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of isochorismate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of Leucyl/phenylalanyl-tRNA-protein transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of b1163-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of cardiolipin synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of tryptophan biosynthesis protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of aldehyde dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of b1346-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of b1522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of b1796-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of methyl-accepting chemotaxis protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of trehalose-phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of imidazole glycerol phosphate synthase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of ATP synthase subunit E in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of putative transport system permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of recombinase A in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of b2739-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of b2849-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of fimbrial-like protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of 3-ketoacyl-CoA thiolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of glutamine synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of b3989-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of b4029-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of b4121-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of RNA binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of choline kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of flavodoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of NAD(P)H-quinone oxidoreductase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of binding-protein-dependent transport systems inner membrane component in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of carbon dioxide concentrating mechanism protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of nitrate/nitrite transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of coproporphyrinogen III oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of precorrin methylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of malate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of geranylgeranyl pyrophosphate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of slr1107-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of photosystem II protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of short-chain alcohol dehydrogenase family in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of Sec-independent protein translocase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of 3-deoxy-7-phosphoheptulonate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of branched-chain amino acid permease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of yfl054c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of glycerol kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of aromatic amino acid aminotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of cystathionine gamma-synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of polygalacturonase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of lipase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of amino acid transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of histone acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of isopentenyl diphosphate isomerase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of cyclin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating one or more activities selected from the group consisting of Zm_{—}4842_BE510522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

An embodiment of the present invention relates to a process for the production of tryptophan, which comprises
(a1) increasing or generating the activity of a polypeptide comprising a polypeptide as depicted in a respective line in column 5 or 8 of Table II, application no. 5, or a homolog or a fragment thereof, a consensus sequence or at least one polypeptide motif as depicted in the respective line in column 8 of Table IV, application no. 5; or
(a2) increasing or generating the activity of an expression product of one or more nucleic acid molecule(s) comprising a polynucleotide as depicted in the respective line in column 5 or 8 of Table I, application no. 5, preferably the coding region thereof, or a homolog or a fragment thereof; or
(a3) increasing or generating the activity of a functional equivalent of (a1) or (a2); in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of tryptophan or a composition comprising tryptophan in said non-human organism or in the culture medium surrounding said non-human organism.

Accordingly, the increase or generation of one or more said activities is for example conferred by one or more expression products of said nucleic acid molecule, e.g. proteins. Accordingly, in the present invention described above, the increase or generation of one or more said activities is for example conferred by one or more protein(s) each comprising a polypeptide selected from the group as depicted in Table II, application no. 5, column 5 and 8, or a homolog or a fragment thereof.

The process of the invention comprises in one embodiment the following steps:
(i) increasing or generating of the expression of; and/or
(ii) increasing or generating the expression of an expression product of; and/or
(iii) increasing or generating one or more activities of an expression product encoded by; at least one nucleic acid molecule (in the following “Fine Chemical Related Protein (FCRP)”-encoding gene or “FCRP”-gene) comprising a nucleic acid molecule selected from the group consisting of:
(a) a nucleic acid molecule encoding the polypeptide shown in column 5 or 8 of Table II, preferably Table II B, application no. 5, or a homolog or a fragment thereof;
(b) a nucleic acid molecule shown in column 5 or 8 of Table I, preferably Table I B, application no. 5, or a homolog or a fragment thereof (preferably the coding region thereof);
(c) a nucleic acid molecule, which, as a result of the degeneracy of the genetic code, can be derived from a polypeptide sequence depicted in column 5 or 8 of Table II, preferably Table II B, application no. 5;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 5, or the coding region thereof;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d),
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 5;
(i) a nucleic acid molecule encoding a polypeptide having the activity represented by a respective protein as depicted in column 5 of Table II, application no. 5;
(j) a nucleic acid molecule which comprises a polynucleotide, which is obtained by amplifying a cDNA library or a genomic library using the primers as depicted in column 8 of Table III, application no. 5; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

The term “increasing or generating of the expression of” may also be interchanged by the term “overexpressing”.

In a preferred embodiment thereof said nucleic acid molecule encodes a polypeptide which has the activity of the polypeptide represented by a protein comprising a polypeptide as depicted in the corresponding hit in column 5 of Table II, application no. 5.

In another preferred embodiment thereof said nucleic acid molecule confers a generation or an increase of the respective fine chemical in a non-human organism or a part thereof as compared to a corresponding wild-type non-human organism or part thereof.

In another preferred embodiment thereof said nucleic acid molecule encodes a polypeptide which has the activity of the polypeptide represented by a protein as depicted in the corresponding hit in column 5 of Table II, application no. 5, and confers a generation or an increase of the respective fine chemical in a non-human organism or a part thereof as compared to a corresponding wild-type non-human organism or part thereof.

Accordingly, the genes of the present invention or used in accordance with the present invention, which respectively encode a protein having an activity of 3-deoxy-7-phosphoheptulonate synthase, 3-ketoacyl-CoA thiolase, acetyltransferase, aldehyde dehydrogenase, amino acid transporter, aromatic amino acid aminotransferase, At1g19800-protein, At2g33510-protein, At4g32480-protein, At5g16650-protein, at5g21910-protein, ATP synthase subunit E, ATP-binding component of a transport system, auxin response factor, b1163-protein, b1346-protein, b1522-protein, b1796-protein, b2739-protein, b2849-protein, b3989-protein, b4029-protein, b4121-protein, binding-protein-dependent transport systems inner membrane component, branched-chain amino acid permease, calcium-dependent protein kinase, carbon dioxide concentrating mechanism protein, cardiolipin synthase, CBL-interacting protein kinase, Chaperone protein CIpB, choline kinase, coproporphyrinogen III oxidase, cullin, cyclin, cyclin D , cystathionine gamma-synthase, DNA mismatch repair protein, electron transfer flavoprotein subunit beta, elongation factor Tu, enoyl-CoA hydratase, eukaryotic translation initiation factor, eukaryotic translation initiation factor 5, fimbrial-like protein, flavodoxin, geranylgeranyl pyrophosphate synthase, gibberellin 20-oxidase, glucose dehydrogenase, glucose-6-phosphate 1-dehydrogenase, glutamate-ammonia-ligase, glutamine synthetase, glutaredoxin, glycerol kinase, glycosyl transferase, harpin-induced family protein, histone acetyltransferase, hydrolase, imidazole glycerol phosphate synthase subunit, isochorismate synthase, isopentenyl diphosphate isomerase, Leucyl/phenylalanyl-tRNA-protein transferase, lipase, L-ribulose-5-phosphate 4-epimerase, malate dehydrogenase, malic enzyme, methyl-accepting chemotaxis protein, methyltransferase, monthiol glutaredoxin, NAD(P)H-quinone oxidoreductase subunit, nitrate/nitrite transport protein, photosystem II protein, polygalacturonase, precorrin methylase, protein kinase, protein phosphatase, putative transport system permease protein, pyruvate kinase, recombinase A, RNA binding protein, RNA-binding protein, Sec-independent protein translocase subunit, short-chain alcohol dehydrogenase family, slr1107-protein, threonine synthase, transaldolase, transcription factor, transcriptional regulator, transport protein, trehalosephosphatase, tryptophan biosynthesis protein, yfl054c-protein, or Zm_{—}4842_BE510522-protein, which respectively encode a protein comprising a polypeptide encoded by a nucleic acid sequence as shown in Table I, application no. 5, column 5 or 8, (preferably the coding region thereof), or a homolog or a fragment thereof, which respectively encode a protein comprising a polypeptide as depicted in Table II, application no. 5, column 5 or 8, or a homolg or a fragment thereof, and/or which respectively can be amplified with the primer set shown in Table III, application no. 5, column 8, are also referred to as “FCRP genes”.

Proteins or polypeptides encoded by “FCRP-genes” are referred to as “Fine Chemical Related Proteins” or “FCRP”. For the purposes of the description of the present invention, the respective protein having an activity of 3-deoxy-7-phosphoheptulonate synthase, 3-ketoacyl-CoA thiolase, acetyltransferase, aldehyde dehydrogenase, amino acid transporter, aromatic amino acid aminotransferase, At1g19800-protein, At2g33510-protein, At4g32480protein, At5g16650-protein, at5g21910-protein, ATP synthase subunit E, ATP-binding component of a transport system, auxin response factor, b1163-protein, b1346-protein, b1522-protein, b1796-protein, b2739-protein, b2849-protein, b3989-protein, b4029-protein, b4121-protein, binding-protein-dependent transport systems inner membrane component, branched-chain amino acid permease, calcium-dependent protein kinase, carbon dioxide concentrating mechanism protein, cardiolipin synthase, CBL-interacting protein kinase, Chaperone protein CIpB, choline kinase, coproporphyrinogen III oxidase, cullin, cyclin, cyclin D , cystathionine gammasynthase, DNA mismatch repair protein, electron transfer flavoprotein subunit beta, elongation factor Tu, enoyl-CoA hydratase, eukaryotic translation initiation factor, eukaryotic translation initiation factor 5, fimbrial-like protein, flavodoxin, geranylgeranyl pyrophosphate synthase, gibberellin 20-oxidase, glucose dehydrogenase, glucose-6-phosphate 1-dehydrogenase, glutamate-ammonia-ligase, glutamine synthetase, glutaredoxin, glycerol kinase, glycosyl transferase, harpin-induced family protein, histone acetyltransferase, hydrolase, imidazole glycerol phosphate synthase subunit, isochorismate synthase, isopentenyl diphosphate isomerase, Leucyl/phenylalanyl-tRNA-protein transferase, lipase, L-ribulose-5-phosphate 4-epimerase, malate dehydrogenase, malic enzyme, methyl-accepting chemotaxis protein, methyltransferase, monthiol glutaredoxin, NAD(P)H-quinone oxidoreductase subunit, nitrate/nitrite transport protein, photosystem II protein, polygalacturonase, precorrin methylase, protein kinase, protein phosphatase, putative transport system permease protein, pyruvate kinase, recombinase A, RNA binding protein, RNA-binding protein, Sec-independent protein translocase subunit, shortchain alcohol dehydrogenase family, slr1107-protein, threonine synthase, transaldolase, transcription factor, transcriptional regulator, transport protein, trehalose-phosphatase, tryptophan biosynthesis protein, yfl054c-protein, or Zm_{—}4842_BE510522-protein, the respective protein comprising a polypeptide encoded by one or more respective nucleic acid sequences as shown in Table I, application no. 5, column 5 or 8, (preferably the coding region thereof), or a homolog or fragment thereof, the respective protein comprising a respective polypeptide as depicted in Table II, application no. 5, column 5 or 8, or a homolog or fragment thereof, the respective protein comprising a sequence corresponding to the consensus sequence as shown in Table IV, application no. 5, column 8, and/or the respective protein comprising at least one polypeptide motif as shown in Table IV, application no. 5, column 8 is also referred to as Fine Chemical Related Protein” or “FCRP”.

Thus, in one embodiment, the present invention provides a process of the production of tryptophan, by increasing or generating one or more activities, especially selected from the group consisting of 3-deoxy-7-phosphoheptulonate synthase, 3-ketoacyl-CoA thiolase, acetyltransferase, aldehyde dehydrogenase, amino acid transporter, aromatic amino acid aminotransferase, At1g19800-protein, At2g33510-protein, At4g32480-protein, At5g16650-protein, at5g21910-protein, ATP synthase subunit E, ATP-binding component of a transport system, auxin response factor, b1163-protein, b1346-protein, b1522-protein, b1796-protein, b2739-protein, b2849-protein, b3989-protein, b4029-protein, b4121-protein, binding-protein-dependent transport systems inner membrane component, branched-chain amino acid permease, calcium-dependent protein kinase, carbon dioxide concentrating mechanism protein, cardiolipin synthase, CBL-interacting protein kinase, Chaperone protein CIpB, choline kinase, coproporphyrinogen III oxidase, cullin, cyclin, cyclin D , cystathionine gamma-synthase, DNA mismatch repair protein, electron transfer flavoprotein subunit beta, elongation factor Tu, enoyl-CoA hydratase, eukaryotic translation initiation factor, eukaryotic translation initiation factor 5, fimbrial-like protein, flavodoxin, geranylgeranyl pyrophosphate synthase, gibberellin 20-oxidase, glucose dehydrogenase, glucose-6-phosphate 1-dehydrogenase, glutamate-ammonia-ligase, glutamine synthetase, glutaredoxin, glycerol kinase, glycosyl transferase, harpin-induced family protein, histone acetyltransferase, hydrolase, imidazole glycerol phosphate synthase subunit, isochorismate synthase, isopentenyl diphosphate isomerase, Leucyl/phenylalanyl-tRNA-protein transferase, lipase, L-ribulose-5-phosphate 4-epimerase, malate dehydrogenase, malic enzyme, methyl-accepting chemotaxis protein, methyltransferase, monthiol glutaredoxin, NAD(P)H-quinone oxidoreductase subunit, nitrate/nitrite transport protein, photosystem II protein, polygalacturonase, precorrin methylase, protein kinase, protein phosphatase, putative transport system permease protein, pyruvate kinase, recombinase A, RNA binding protein, RNA-binding protein, Sec-independent protein translocase subunit, short-chain alcohol dehydrogenase family, slr1107-protein, threonine synthase, transaldolase, transcription factor, transcriptional regulator, transport protein, trehalose-phosphatase, tryptophan biosynthesis protein, yfl054c-protein, and Zm_{—}4842_BE510522-protein, which is conferred by one or more FCRPs or the gene product of one or more FCRP-genes, for example by the gene product of a nucleic acid sequences comprising a polynucleotide selected from the group as shown in Table I, application no. 5, column 5 or 8, (preferably by the coding region thereof), or a homolog or a fragment thereof, e.g. or by one or more proteins each comprising a polypeptide encoded by one or more nucleic acid sequences selected from the group as shown in Table I, application no. 5, column 5 or 8, (preferably by the coding region thereof), or a homolog or a fragment thereof, or by one or more protein(s) each comprising a polypeptide selected from the group as depicted in Table II, application no. 5, column 5 and 8, or a homolog thereof, or a protein comprising a sequence corresponding to the consensus sequence or comprising at least one polypeptide motif as shown in Table IV, application no. 5, column 8.

for the disclosure of this paragraph see [0025.1.1.1] above

In an embodiment, the process comprises increasing or generating the activity of one or more polypeptides having said activity, e.g. by generating or increasing the amount and/or specific activity in the cell or a compartment of a cell of one of more FCRP, especially selected from the group consisting of 3-deoxy-7-phosphoheptulonate synthase, 3-ketoacyl-CoA thiolase, acetyltransferase, aldehyde dehydrogenase, amino acid transporter, aromatic amino acid aminotransferase, At1g19800-protein, At2g33510-protein, At4g32480-protein, At5g16650-protein, at5g21910-protein, ATP synthase subunit E, ATP-binding component of a transport system, auxin response factor, b1163-protein, b1346-protein, b1522-protein, b1796-protein, b2739-protein, b2849-protein, b3989-protein, b4029-protein, b4121-protein, binding-protein-dependent transport systems inner membrane component, branched-chain amino acid permease, calcium-dependent protein kinase, carbon dioxide concentrating mechanism protein, cardiolipin synthase, CBL-interacting protein kinase, Chaperone protein CIpB, choline kinase, coproporphyrinogen III oxidase, cullin, cyclin, cyclin D , cystathionine gamma-synthase, DNA mismatch repair protein, electron transfer flavoprotein subunit beta, elongation factor Tu, enoyl-CoA hydratase, eukaryotic translation initiation factor, eukaryotic translation initiation factor 5, fimbrial-like protein, flavodoxin, geranylgeranyl pyrophosphate synthase, gibberellin 20-oxidase, glucose dehydrogenase, glucose-6-phosphate 1-dehydrogenase, glutamate-ammonia-ligase, glutamine synthetase, glutaredoxin, glycerol kinase, glycosyl transferase, harpin-induced family protein, histone acetyltransferase, hydrolase, imidazole glycerol phosphate synthase subunit, isochorismate synthase, isopentenyl diphosphate isomerase, Leucyl/phenylalanyl-tRNA-protein transferase, lipase, L-ribulose-5-phosphate 4-epimerase, malate dehydrogenase, malic enzyme, methyl-accepting chemotaxis protein, methyltransferase, monthiol glutaredoxin, NAD(P)H-quinone oxidoreductase subunit, nitrate/nitrite transport protein, photosystem II protein, polygalacturonase, precorrin methylase, protein kinase, protein phosphatase, putative transport system permease protein, pyruvate kinase, recombinase A, RNA binding protein, RNA-binding protein, Sec-independent protein translocase subunit, short-chain alcohol dehydrogenase family, slr1107-protein, threonine synthase, transaldolase, transcription factor, transcriptional regulator, transport protein, trehalosephosphatase, tryptophan biosynthesis protein, yfl054c-protein, and Zm_{—}4842_BE510522protein, for example of the respective polypeptide as depicted in Table II, application no. 5, column 5 and 8, or a homolog or a fragment thereof, or the respective polypeptide comprising a sequence corresponding to the consensus sequences as shown in Table IV, application no. 5, column 8, or the respective polypeptide comprising at least one polypeptide motif as depicted in Table IV, application no. 5, column 8.

A further embodiment of the present invention relates to a process for the production of tryptophan, which comprises
(a) increasing or generating the activity of a glucose-6-phosphate 1-dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a At1g19800-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a cullin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a eukaryotic translation initiation factor 5 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a CBL-interacting protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a At2g33510-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a harpin-induced family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a DNA mismatch repair protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a eukaryotic translation initiation factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a glycosyl transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a auxin response factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a monthiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a At4g32480-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a gibberellin 20-oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a At5g16650-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a at5g21910-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a RNA-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a malic enzyme non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a elongation factor Tu non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a enoyl-CoA hydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a enoyl-CoA hydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a glutamate-ammonia-ligase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a Chaperone protein CIpB non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a electron transfer flavoprotein subunit beta non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a threonine synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a transaldolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a L-ribulose-5-phosphate 4-epimerase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a glucose dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a ATP-binding component of a transport system non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a isochorismate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a Leucyl/phenylalanyl-tRNA-protein transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a b1163-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a cardiolipin synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a tryptophan biosynthesis protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a aldehyde dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a b1346-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a b1522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a b1796-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a methyl-accepting chemotaxis protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a trehalose-phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a imidazole glycerol phosphate synthase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a ATP synthase subunit E non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a putative transport system permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a recombinase A non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a b2739-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a b2849-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a fimbrial-like protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a 3-ketoacyl-CoA thiolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a glutamine synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a b3989-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a b4029-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a b4121-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a RNA binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a choline kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a flavodoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a NAD(P)H-quinone oxidoreductase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a binding-protein-dependent transport systems inner membrane component non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a carbon dioxide concentrating mechanism protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a nitrate/nitrite transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a coproporphyrinogen III oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a precorrin methylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a malate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a geranylgeranyl pyrophosphate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a slr1107-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a photosystem II protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a short-chain alcohol dehydrogenase family non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a Sec-independent protein translocase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a 3-deoxy-7-phosphoheptulonate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a branched-chain amino acid permease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a yfl054c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a glycerol kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a aromatic amino acid aminotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a cystathionine gamma-synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a polygalacturonase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a lipase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a amino acid transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a histone acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a isopentenyl diphosphate isomerase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a cyclin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a Zm_{—}4842_BE510522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating the activity of a polypeptide comprising a polypeptide as depicted in the respective line in column 5 or 8 of Table II, application no. 5, or a homolog or a fragment thereof, a consensus sequence or at least one polypeptide motif as depicted in the respective line in column 8 of Table IV, application no. 5; or increasing or generating the activity of an expression product of one or more nucleic acid molecule(s) comprising a polynucleotide as depicted in the respective line in column 5 or 8 of Table I, application no. 5, preferably the coding region thereof, or a homolog or a fragment thereof;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of tryptophan, or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of glucose-6-phosphate 1-dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of At1g19800-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of cullin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of eukaryotic translation initiation factor 5 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of CBL-interacting protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of At2g33510-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of harpin-induced family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of DNA mismatch repair protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of eukaryotic translation initiation factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of glycosyl transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of auxin response factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of monthiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of At4g32480-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of gibberellin 20-oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of At5g16650-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of at5g21910-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of RNA-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of malic enzyme in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of elongation factor Tu in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of enoyl-CoA hydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of enoyl-CoA hydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of glutamate-ammonia-ligase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of Chaperone protein CIpB in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of electron transfer flavoprotein subunit beta in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of threonine synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of transaldolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of L-ribulose-5-phosphate 4-epimerase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of glucose dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of ATP-binding component of a transport system in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of isochorismate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of Leucyl/phenylalanyl-tRNA-protein transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of b1163-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of cardiolipin synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of tryptophan biosynthesis protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of aldehyde dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of b1346-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of b1522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of b1796-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of methyl-accepting chemotaxis protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of trehalose-phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of imidazole glycerol phosphate synthase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of ATP synthase subunit E in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of putative transport system permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of recombinase A in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of b2739-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of b2849-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of fimbrial-like protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of 3ketoacyl-CoA thiolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of glutamine synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of b3989-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of b4029-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of b4121-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of RNA binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of choline kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of flavodoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of NAD(P)H-quinone oxidoreductase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of binding-protein-dependent transport systems inner membrane component in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of carbon dioxide concentrating mechanism protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of nitrate/nitrite transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of coproporphyrinogen III oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of precorrin methylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of malate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of geranylgeranyl pyrophosphate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of slr1107-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of photosystem II protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of short-chain alcohol dehydrogenase family in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of Sec-independent protein translocase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of 3-deoxy-7-phosphoheptulonate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of branched-chain amino acid permease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of yfl054c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of glycerol kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of aromatic amino acid aminotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of cystathionine gamma-synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of polygalacturonase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of lipase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of amino acid transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of histone acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of isopentenyl diphosphate isomerase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of cyclin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a) increasing or generating one or more activities selected from the group consisting of Zm_{—}4842_BE510522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

A further embodiment of the present invention relates to a process for the production of tryptophan, which comprises
(a1) increasing or generating the activity of a polypeptide comprising a polypeptide as depicted in the respective line in column 5 or 8 of Table II, application no. 5, or a homolog or fragment thereof, a consensus sequence or at least one polypeptide motif as depicted in column 8 of Table IV, application no. 5; or increasing or generating the activity of an expression product of one or more nucleic acid molecule(s) comprising a polynucleotide as depicted in the respective line in column 5 or 8 of Table I, application no. 5, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 5, or a homolog or a fragment thereof, a consensus sequence or at least one polypeptide motif as depicted in the respective line in column 8 of Table IV, application no. 5, which is joined to a transit peptide; or
increasing or generating the activity of an expression product of one or more nucleic acid molecule(s) comprising a polynucleotide as depicted in the respective line in column 5 or 8 of Table I, application no. 5, preferably the coding region thereof, or a homolog or a fragment thereof, which is joined to a nucleic acid sequence encoding an organelle localization sequence, preferably a plastid or a mitochondrion localization sequence, especially a plastid localization sequence;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 5, or a homolog or a fragment thereof, a consensus sequence or at least one polypeptide motif as depicted in the respective line in column 8 of Table IV, application no. 5; or increasing or generating the activity of an expression product of one or more nucleic acid molecule(s) comprising a polynucleotide as depicted in the respective line in column 5 or 8 of Table I, application no. 5, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of tryptophan, or a composition comprising tryptophan in said non-human organism or in the culture medium surrounding said non-human organism.

Accordingly, the present invention relates to a process for the production of tryptophan, which comprises
(a) increasing or generating the activity of a glucose-6-phosphate 1-dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a At1g19800-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a cullin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a eukaryotic translation initiation factor 5 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a CBL-interacting protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a At2g33510-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a harpin-induced family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a DNA mismatch repair protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a eukaryotic translation initiation factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a glycosyl transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a auxin response factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a monthiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a At4g32480-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a gibberellin 20-oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a At5g16650-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a at5g21910-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a RNA-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a malic enzyme in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a elongation factor Tu in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a enoyl-CoA hydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a enoyl-CoA hydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a glutamate-ammonia-ligase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a Chaperone protein CIpB in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a electron transfer flavoprotein subunit beta in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a threonine synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a transaldolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a L-ribulose-5-phosphate 4-epimerase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a glucose dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a ATP-binding component of a transport system in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a isochorismate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a Leucyl/phenylalanyl-tRNA-protein transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a b1163-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a cardiolipin synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a tryptophan biosynthesis protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a aldehyde dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a b1346-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a b1522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a b1796-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a methyl-accepting chemotaxis protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a trehalose-phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a imidazole glycerol phosphate synthase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a ATP synthase subunit E in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a putative transport system permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a recombinase A in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a b2739-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a b2849-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a fimbrial-like protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a 3-ketoacyl-CoA thiolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a glutamine synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a b3989-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a b4029-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a b4121-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a RNA binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a choline kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a flavodoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a NAD(P)H-quinone oxidoreductase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a binding-protein-dependent transport systems inner membrane component in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a carbon dioxide concentrating mechanism protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a nitrate/nitrite transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a coproporphyrinogen III oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a precorrin methylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a malate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a geranylgeranyl pyrophosphate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a slr1107-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a photosystem II protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a short-chain alcohol dehydrogenase family in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a Sec-independent protein translocase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a 3-deoxy-7-phosphoheptulonate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a branched-chain amino acid permease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a yfl054c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a glycerol kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a aromatic amino acid aminotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a cystathionine gamma-synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a polygalacturonase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a lipase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a amino acid transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a histone acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a isopentenyl diphosphate isomerase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a cyclin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a Zm_{—}4842_BE510522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Accordingly, the present invention relates to a process for the production of tryptophan, which comprises
(a) increasing or generating the activity of a polypeptide comprising a polypeptide as depicted in the respective line in column 5 or 8 of Table II, application no. 5, or a homolog or a fragment thereof, a consensus sequence or at least one polypeptide motif as depicted in the respective line in column 8 of Table IV, application no. 5; or increasing or generating the activity of an expression product of one or more nucleic acid molecule(s) comprising a polynucleotide as depicted in the respective line in column 5 or 8 of Table I, application no. 5, preferably the coding region thereof, or a homolog or a fragment thereof;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of tryptophan, or a composition comprising tryptophan in said non-human organism or in the culture medium surrounding said non-human organism.

In a further embodiment the activity of the polypeptide comprising a polypeptide as depicted in the respective line in column 5 or 8 of Table II, application no. 5, or a homolog or a fragment thereof, a consensus sequence or at least one polypeptide motif as depicted in the respective line in column 8 of Table IV, application no. 5, is increased or generated non-targeted in the above-mentioned process in a microorganism or plant or a part thereof.

In a further embodiment said polypeptide has the activity of the respective polypeptide represented by a protein comprising a polypeptide as depicted in the respective line in column 5 of Table II, application no. 5.

In a further embodiment the activity of the expression product of one or more nucleic acid molecule(s) comprising a polynucleotide as depicted in the respective line in column 5 or 8 of Table I, application no. 5, preferably the coding region thereof, or a homolog or a fragment thereof, is increased or generated non-targeted in the above-mentioned process in a microorganism or plant or a part thereof.

In a further embodiment the activity of the polypeptide comprising a polypeptide as depicted in the respective line in column 5 or 8 of Table II, application no. 5, or a homolog or a fragment thereof, a consensus sequence or at least one polypeptide motif as depicted in the respective line in column 8 of Table IV, application no. 5, is increased or generated in the above-mentioned process in an organelle preferably in plastids or mitochondria, especially in plastids, of a microorganism or plant.

In a further embodiment said polypeptide has the activity of the respective polypeptide represented by a protein comprising a polypeptide as depicted in the respective line in column 5 of Table II, application no. 5.

In a further embodiment the activity of the expression product of one or more nucleic acid molecule(s) comprising a polynucleotide as depicted in the respective line in column 5 or 8 of Table I, application no. 5, preferably the coding region thereof, or a homolog or a fragment thereof, is increased or generated in the above-mentioned process in an organelle, preferably in plasids or mitochondria, especially in plastids, of a microorganism or plant.

In a further embodiment the activity of the polypeptide comprising a polypeptide as depicted in the respective line in column 5 or 8 of Table II, application no. 5, or a homolog or a fragment thereof, a consensus sequence or at least one polypeptide motif as depicted in the respective line in column 8 of Table IV, application no. 5, is increased or generated in the above-mentioned process in the cyctosol of a cell, of a microorganism or plant.

In a further embodiment said polypeptide has the activity of the respective polypeptide represented by a protein comprising a polypeptide as depicted in the respective line in column 5 of Table II, application no. 5.

In a further embodiment the activity of the expression product of one or more nucleic acid molecule(s) comprising a polynucleotide as depicted in the respective line in column 5 or 8 of Table I, application no. 5, preferably the coding region thereof, or a homolog or a fragment thereof, is increased or generated in the above-mentioned process in the cytosol of a cell, of a microorganism or plant.

[0039.1.1.5] to [0066.1.1.5] for the disclosure of these paragraphs see [0039.1.1.1] to [0066.1.1.1] above.

As mentioned above the nucleic acid sequences coding for the proteins as shown in Table II, application no. 5, column 5 or 8, or homologs or fragments thereof, are joined to a nucleic acid sequence encoding a plastidic transit peptide, e.g, if for the nucleic acid molecule in Table I, column 6 the term “plastidic” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in Table II, application no. 5, column 5 or 8, or homologs or fragments thereof, e.g., if for the nucleic acid molecule in Table I, column 6 the term “plastidic” is indicated.

[0068.1.1.5] to [0072.1.1.5] for the disclosure of these paragraphs see [0068.1.1.1] to [0072.1.1.1] above.

As mentioned above the nucleic acid sequences coding for the proteins as shown in the respective line in Table II, application no. 5, column 5 or 8, or homologs or fragments thereof, are joined to a nucleic acid sequence encoding a mitochondric transit peptide, e.g., if for the respective nucleic acid molecule in Table I, column 6 the term “mitochondrial” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in the respective line in Table II, application no. 5, column 5 or 8, or homologs or fragments thereof, e.g., if for the nucleic acid molecule in the respective line in Table I, column 6 the term “mitochondrial” is indicated. (For the avoidance of doubt, the term “mitochondric” and “mitochondrial” are interchangeable.)

[0074.1.1.5] to [0075.1.1.5] for the disclosure of these paragraphs see [0074.1.1.1] to [0075.1.1.1] above

Nucleic acid sequences coding for the transit peptides may be chemically synthesized either in part or wholly according to structure of transit peptide sequences disclosed in the prior art. Said natural or chemically synthesized sequences can be directly linked to the sequences encoding the mature protein or via a linker nucleic acid sequence, which may be typically less than 500 base pairs, preferably less than 450, 400, 350, 300, 250 or 200 base pairs, more preferably less than 150, 100, 90, 80, 70, 60, 50, 40 or 30 base pairs and most preferably less than 25, 20, 15, 12, 9, 6 or 3 base pairs in length and are in frame to the coding sequence. Furthermore favorable nucleic acid sequences encoding transit peptides may comprise sequences derived from more than one biological and/or chemical source and may include a nucleic acid sequence derived from the amino-terminal region of the mature protein, which in its native state is linked to the transit peptide. In a preferred embodiment of the invention said amino-terminal region of the mature protein is typically less than 150 amino acids, preferably less than 140, 130, 120, 110, 100 or 90 amino acids, more preferably less than 80, 70, 60, 50, 40, 35, 30, 25 or 20 amino acids and most preferably less than 19, 18, 17, 16, 15, 14, 13, 12, 11 or 10 amino acids in length. But even shorter or longer stretches are also possible. In addition target sequences, which facilitate the transport of proteins to other cell compartments such as the vacuole, endoplasmic reticulum, golgi complex, glyoxysomes, peroxisomes, mitochondria (in case of a plastidal transit peptide) or plastids, especially chloroplasts (in case of a mitochondric transit peptide) may be also part of the inventive nucleic acid sequence. The proteins translated from said inventive nucleic acid sequences are a kind of fusion proteins that means the nucleic acid sequences encoding the plastidal transit peptide for example the ones shown in Table a, preferably the last one of the Table a are joint to the respective nucleic acid sequences shown in Table I, application no. 5, columns 5 or 8, especially the coding region thereof, or homologs or fragments thereof, in case of nucleic acid sequences being expressed in plastids, and that means the nucleic acid sequences encoding the mitochondric transit peptide for example the ones shown in Table b, preferably the last one of the Table b are joint to the respective nucleic acid sequences shown in Table I, application no. 5, columns 5 or 8, especially the coding region thereof, or homologs or fragments thereof, in case of nucleic acid sequences being expressed in mitochondria. The person skilled in the art is able to join said sequences in a functional manner. Advantageously the transit peptide part is cleaved off from the protein part shown in Table II, application no. 5, columns 5 or 8, or a homolog or fragment thereof, during or after the transport into the organelles, preferably into the plastids or mitochondria, respectively.

All products of the cleavage of the preferred transit peptide shown in the last line of Table a have preferably the N-terminal amino acid sequences QIA CSS or QIA EFQLTT in front of the start methionine of the respective protein mentioned in Table II, application no. 5, column 5 or 8. or homologs or fragments thereof. Other short amino acid sequences of an range of 1 to 20 amino acids preferable 2 to 15 amino acids, more preferable 3 to 10 amino acids most preferably 4 to 8 amino acids are also possible in front of the start methionine of the protein mentioned in Table II, application no. 5, columns 5 or 8, or homologs or fragments thereof. In case of the amino acid sequence QIA CSS the three amino acids in front of the start methionine are stemming from the LIC (=ligatation independent cloning) cassette. Said short amino acid sequence is preferred in the case of the expression of E. coli genes. In case of the amino acid sequence QIA EFQLTT the six amino acids in front of the start methionine are stemming from the LIC cassette. Said short amino acid sequence is preferred in the case of the expression of S. cerevisiae genes. The skilled worker knowns that other short sequences are also useful in the expression of the respective nucleic acid molecule comprising a polynucleotide as depicted in Table I, application no. 5, column 5 or 8, preferably the coding region thereof, or a homolog or a fragment thereof. Furthermore the skilled worker is aware of the fact that there is not a need for such short sequences in the expression of the genes.

[0077.1.1.5]to [0078.1.1.5] for the disclosure of these paragraphs see [0077.1.1.1] to [0078.1.1.1] above.

Alternatively to the targeting of the respective sequences shown in Table II, application no. 5, columns 5 or 8, or homologs or fragments thereof, preferably of sequences in general encoded in the nucleus with the aid of the targeting sequences mentioned for example in Table a alone, Table b alone, or in combination with other targeting sequences preferably into plastids or mitochondria, respectively, the nucleic acids of the invention can directly be introduced into the plastidal or mitochondrial genome. Therefore in a preferred embodiment the respective nucleic acid sequences shown in the respective line in Table I, application no. 5, columns 5 or 8, preferably the coding region thereof, or homologs or fragments thereof, are directly introduced and expressed in plastids e.g., if for the nucleic acid molecule in the respective line in Table I, column 6, the term “plastidic” is indicated, or are directly introduced and expressed in mitochondria e.g., if for the nucleic acid molecule in the respective line in Table I, column 6 the term “mitochondrial” is indicated.

The term “introduced” in the context of this specification shall mean the insertion of a nucleic acid sequence into the organism by means of a “transfection”, “transduction” or preferably by “transformation”.

A plastid, such as a chloroplast, or a mitochondrion, has been “transformed” by an exogenous (preferably foreign) nucleic acid sequence if a nucleic acid sequence has been introduced into the plastid or mitochondrion that means that this sequence has crossed the membrane or the membranes of the plastid or of the mitochondrion. The exogenous, preferably foreign, DNA may be integrated (covalently linked) into plastid or mitochondrial DNA making up the genome of the plastid or mitochondrion, or it may remain unintegrated (e.g., by including a chloroplast origin of replication). “Stably” integrated DNA sequences are those, which are inherited through plastid or mitochondrion replication, thereby transferring new plastids or mitochondria, with the features of the integrated DNA sequence to the progeny.

[0080.1.1.5] to [0083.1.1.5] for the disclosure of these paragraphs see [0080.1.1.1] to [0083.1.1.1] above.

For the process of the present invention it may be of great advantage that by transforming the plastids the intraspecies specific transgene flow is blocked, because a lot of species such as corn, cotton and rice have a strict maternal inheritance of plastids. By placing the respective genes specified in the respective line in Table I, application no. 5, columns 5 or 8, preferably the coding region, or homologs or fragments thereof, e.g. if for the nucleic acid molecule in the respective line in column 6 of Table I the term “plastidic” is indicated, in the plastids of plants, these genes will not be present in the pollen of said plants.

A further preferred embodiment of the invention relates to the use of so called “chloroplast localization sequences”, in which a first RNA sequence or molecule is capable of transporting or “chaperoning” a second RNA sequence, such as a RNA sequence transcribed from the sequences depicted in the respective line in Table I, application no. 5, columns 5 or 8, preferably the coding region thereof, or a homolog or a fragment thereof, or a sequence encoding a protein, as depicted in the respective line in Table II, application no. 5, columns 5 and 8, or a homolog or a fragment thereof, from an external environment inside a cell or outside a plastid into a chloroplast. In one embodiment the chloroplast localization signal is substantially similar or complementary to a complete or intact viroid sequence. The chloroplast localization signal may be encoded by a DNA sequence, which is transcribed into the chloroplast localization RNA. The term “viroid” refers to a naturally occurring single stranded RNA molecule (Flores, C. R. Acad. Sci. III. 324 (10), 943 (2001)). Viroids usually contain about 200-500 nucleotides and generally exist as circular molecules. Examples of viroids that contain chloroplast localization signals include but are not limited to ASBVd, PLMVd, CChMVd and ELVd. The viroid sequence or a functional part of it can be fused to the sequences depicted in the respective line in Table I, application no. 5, columns 5 or 8, preferably the coding region thereof, or a homolog or a fragment thereof, or a sequence encoding a protein, as depicted in the respective line in Table II, application no. 5, columns 5 or 8, or a homolog or a fragment thereof, in such a manner that the viroid sequence transports a sequence transcribed from a sequence as depicted in the respective line in Table I, application no. 5 columns 5 or 8, preferably the coding region thereof, or a homolog or a fragment thereof, or a sequence encoding a protein as depicted in the respective line in Table II, application no. 5 columns 5 or 8, or a homolog or a fragment thereof, into the chloroplasts, e.g. if for the nucleic acid molecule in column 6 of Table I the term “plastidic” is indicated. A preferred embodiment uses a modified ASBVd (Navarro et al., Virology. 268 (1), 218 (2000)).

In a further specific embodiment the protein to be expressed in the plastids such as the proteins depicted in the respective line in Table II, application no. 5, columns 5 or 8, or a homolog or a fragment thereof, e.g. if for the nucleic acid molecule in the respective line in column 6 of Table I the term “plastidic” is indicated, are encoded by different nucleic acids. Such a method is disclosed in WO 2004/040973, which shall be incorporated by reference. WO 2004/040973 teaches a method, which relates to the translocation of an RNA corresponding to a gene or gene fragment into the chloroplast by means of a chloroplast localization sequence. The genes, which should be expressed in the plant or plant cells, are split into nucleic acid fragments, which are introduced into different compartments in the plant e.g. the nucleus, the plastids and/or mitochondria. Additionally plant cells are described in which the chloroplast contains a ribozyme fused at one end to an RNA encoding a fragment of a protein used in the inventive process such that the ribozyme can trans-splice the translocated fusion RNA to the RNA encoding the gene fragment to form and as the case may be reunite the nucleic acid fragments to an intact mRNA encoding a functional protein for example as disclosed in the respective line in Table II, application no. 5, columns 5 or 8, or a homolog or a fragment thereof.

In a preferred embodiment of the invention the nucleic acid sequences as shown in the respective line in Table I, application no. 5, columns 5 or 8, preferably the coding region thereof, or homologs or fragments thereof, e.g. if for the nucleic acid molecule in the respective line in column 6 of Table I the term “plastidic” is indicated, used in the inventive process are transformed into plastids, which are metabolically active. Those plastids should preferably maintain at a high copy number in the plant or plant tissue of interest, most preferably the chloroplasts found in green plant tissues, such as leaves or cotyledons or in seeds.

For a good expression in the plastids the nucleic acid sequences as shown in the respective line in Table I, application no. 5, columns 5 or 8, preferably the coding region thereof, or homologs or fragments thereof, e.g. if for the nucleic acid molecule in the respective line in column 6 of Table I the term “plastidic” is indicated, are introduced into an expression cassette using preferably a promoter and terminater, which are active in plastids preferably a chloroplast promoter. Examples of such promoters include the psbA promoter from the gene from spinach or pea, the rbcL promoter, and the atpB promoter from corn.

[0089.1.1.5] to [0092.1.1.5] for the disclosure of these paragraphs see [0089.1.1.1] to [0092.1.1.1] above.

Advantageously the process for the production of the fine chemical tryptophan leads to an enhanced production of the respective fine chemical. The terms “enhanced” or “increase” mean at least a 10%, 20%, 30%, 40% or 50%, preferably at least 60%, 70%, 80%, 90% or 100%, more preferably 150%, 200%, 300%, 400% or 500% higher production of the respective fine chemical tryptophan in comparison to the wild-type as defined above, e.g. that means in comparison to a non-human organism without the aforementioned modification of the activity of a protein as shown in the respective line in Table II, application no. 5, column 5 or 8, or a fragment or a homolog thereof. The modification of the activity of a protein as shown in the respective line in Table II, application no. 5, column 5 or 8, or a homolog or a fragment thereof, or their combination can be achieved by joining the protein to a respective transit peptide, e.g. if for the respective encoding nucleic acid molecule in column 6 of Table I the term “plastidic” or “mitochondrial” is indicated.

Surprisingly it was found, that the transgenic expression of a protein as shown in the respective line in Table II, application no. 5, column 5, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, conferred a production of or an increase in tryptophan,respectively, to the transgenic non-human organism as compared to a corresponding non-transformed wild type.

In one embodiment the transgenic expression of a protein as shown in the respective line in Table II, application no. 4, column 5 or 8, or a homolog or a fragment thereof, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, conferred a production of or an increase in threonine,respectively, to the transgenic non-human organism as compared to a corresponding non-transformed wild type.

Accordingly, in one embodiment, an increase of tryptophan in a non-human organism, as compared to a corresponding non-transformed wild type non-human organism, is conferred in the process of the invention, if the activity of a polypeptide showing the activity of a glucose-6-phosphate 1-dehydrogenase, or if the activity of the polypeptide 59582753_SOYBEAN, preferably represented by SEQ ID NO. 16884, or a homolog or fragment thereof, or if the activity of a polypeptide encoded by a nucleic acid molecule comprising the nucleic acid SEQ ID NO. 16883, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 16883 or polypeptide SEQ ID NO. 16884, respectively, or a homolog or a fragment thereof, is increased or generated, or if the activity glucose-6-phosphate 1-dehydrogenase is increased or generated in a non-human organism, like a microorganism or a plant cell, plant or part thereof, especially with plastidic localization, For example, an increase of the tryptophan of at least 1 percent, particularly in a range of 28 to 92-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a At1g19800-protein, or if the activity of the polypeptide At1g19800, preferably represented by SEQ ID NO. 17638, or a homolog or fragment thereof, or if the activity of a polypeptide encoded by a nucleic acid molecule comprising the nucleic acid SEQ ID NO. 17637, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 17637 or polypeptide SEQ ID NO. 17638, respectively, or a homolog or a fragment thereof, is increased or generated, or if the activity At1g19800-protein is increased or generated in a non-human organism, like a microorganism or a plant cell, plant or part thereof, especially with non-targeted localization, For example, an increase of the tryptophan of at least 1 percent, particularly in a range of 30 to 443-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a cullin, or if the activity of the polypeptide At1g26830, preferably represented by SEQ ID NO. 17702, or a homolog or fragment thereof, or if the activity of a polypeptide encoded by a nucleic acid molecule comprising the nucleic acid SEQ ID NO. 17701, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 17701 or polypeptide SEQ ID NO. 17702, respectively, or a homolog or a fragment thereof, is increased or generated, or if the activity cullin is increased or generated in a non-human organism, like a microorganism or a plant cell, plant or part thereof, especially with non-targeted localization, For example, an increase of the tryptophan of at least 1 percent, particularly in a range of 32 to 117-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a eukaryotic translation initiation factor 5, or if the activity of the polypeptide At1g36730, preferably represented by SEQ ID NO. 17969, or a homolog or fragment thereof, or if the activity of a polypeptide encoded by a nucleic acid molecule comprising the nucleic acid SEQ ID NO. 17968, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 17968 or polypeptide SEQ ID NO. 17969, respectively, or a homolog or a fragment thereof, is increased or generated, or if the activity eukaryotic translation initiation factor 5 is increased or generated in a non-human organism, like a microorganism or a plant cell, plant or part thereof, especially with non-targeted localization, For example, an increase of the tryptophan of at least 1 percent, particularly in a range of 39 to 78-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a protein kinase, or if the activity of the polypeptide At1g48260, preferably represented by SEQ ID NO. 18236, or a homolog or fragment thereof, or if the activity of a polypeptide encoded by a nucleic acid molecule comprising the nucleic acid SEQ ID NO. 18235, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 18235 or polypeptide SEQ ID NO. 18236, respectively, or a homolog or a fragment thereof, is increased or generated, or if the activity protein kinase is increased or generated in a non-human organism, like a microorganism or a plant cell, plant or part thereof, especially with non-targeted localization, For example, an increase of the tryptophan of at least 1 percent, particularly in a range of 36 to 239-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a transcription factor, or if the activity of the polypeptide At2g17560, preferably represented by SEQ ID NO. 19503, or a homolog or fragment thereof, or if the activity of a polypeptide encoded by a nucleic acid molecule comprising the nucleic acid SEQ ID NO. 19502, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 19502 or polypeptide SEQ ID NO. 19503, respectively, or a homolog or a fragment thereof, is increased or generated, or if the activity transcription factor is increased or generated in a non-human organism, like a microorganism or a plant cell, plant or part thereof, especially with non-targeted localization, For example, an increase of the tryptophan of at least 1 percent, particularly in a range of 34 to 179 -percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a protein phosphatase, or if the activity of the polypeptide At2g28890, preferably represented by SEQ ID NO. 19875, or a homolog or fragment thereof, or if the activity of a polypeptide encoded by a nucleic acid molecule comprising the nucleic acid SEQ ID NO. 19874, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 19874 or polypeptide SEQ ID NO. 19875, respectively, or a homolog or a fragment thereof, is increased or generated, or if the activity protein phosphatase is increased or generated in a non-human organism, like a microorganism or a plant cell, plant or part thereof, especially with non-targeted localization, For example, an increase of the tryptophan of at least 1 percent, particularly in a range of 32 to 330-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a CBL-interacting protein kinase, or if the activity of the polypeptide At2g30360, preferably represented by SEQ ID NO. 19920, or a homolog or fragment thereof, or if the activity of a polypeptide encoded by a nucleic acid molecule comprising the nucleic acid SEQ ID NO. 19919, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 19919 or polypeptide SEQ ID NO. 19920, respectively, or a homolog or a fragment thereof, is increased or generated, or if the activity CBL-interacting protein kinase is increased or generated in a non-human organism, like a microorganism or a plant cell, plant or part thereof, especially with non-targeted localization, For example, an increase of the tryptophan of at least 1 percent, particularly in a range of 46 to 700-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a glutaredoxin, or if the activity of the polypeptide At2g30540, preferably represented by SEQ ID NO. 20347, or a homolog or fragment thereof, or if the activity of a polypeptide encoded by a nucleic acid molecule comprising the nucleic acid SEQ ID NO. 20346, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 20346 or polypeptide SEQ ID NO. 20347, respectively, or a homolog or a fragment thereof, is increased or generated, or if the activity glutaredoxin is increased or generated in a non-human organism, like a microorganism or a plant cell, plant or part thereof, especially with non-targeted localization, For example, an increase of the tryptophan of at least 1 percent, particularly in a range of 28 to 126-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a At2g33510protein, or if the activity of the polypeptide At2g33510, preferably represented by SEQ ID NO. 98523, or a homolog or fragment thereof, or if the activity of a polypeptide encoded by a nucleic acid molecule comprising the nucleic acid SEQ ID NO. 98522, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 98522 or polypeptide SEQ ID NO. 98523, respectively, or a homolog or a fragment thereof, is increased or generated, or if the activity At2g33510-protein is increased or generated in a non-human organism, like a microorganism or a plant cell, plant or part thereof, especially with non-targeted localization, For example, an increase of the tryptophan of at least 1 percent, particularly in a range of 34 to 59-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a glutaredoxin, or if the activity of the polypeptide At2g47880, preferably represented by SEQ ID NO. 21160, or a homolog or fragment thereof, or if the activity of a polypeptide encoded by a nucleic acid molecule comprising the nucleic acid SEQ ID NO. 21159, preferably the coding region thereof, or a homolog or fragment thereof, e.g. derived from Arabidopsis thaliana, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 21159 or polypeptide SEQ ID NO. 21160, respectively, or a homolog or a fragment thereof, is increased or generated, or if the activity glutaredoxin is increased or generated in a non-human organism, like a microorganism or a plant cell, plant or part thereof, especially with non-targeted localization, For example, an increase of the tryptophan of at least 1 percent, particularly in a range of 33 to 380-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a pyruvate kinase, or if the activity of the polypeptide At3g04050, preferably represented by SEQ ID NO. 21498, or a homolog or fragment thereof, or if the activity of a polypeptide encoded by a nucleic acid molecule comprising the nucleic acid SEQ ID NO. 21497, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 21497 or polypeptide SEQ ID NO.

21498, respectively, or a homolog or a fragment thereof, is increased or generated, or if the activity pyruvate kinase is increased or generated in a non-human organism, like a microorganism or a plant cell, plant or part thereof, especially with plastidic localization, For example, an increase of the tryptophan of at least 1 percent, particularly in a range of 76 to 131-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a harpin-induced family protein, or if the activity of the polypeptide At3g11650, preferably represented by SEQ ID NO. 22612, or a homolog or fragment thereof, or if the activity of a polypeptide encoded by a nucleic acid molecule comprising the nucleic acid SEQ ID NO. 22611, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 22611 or polypeptide SEQ ID NO. 22612, respectively, or a homolog or a fragment thereof, is increased or generated, or if the activity harpin-induced family protein is increased or generated in a non-human organism, like a microorganism or a plant cell, plant or part thereof, especially with non-targeted localization, For example, an increase of the tryptophan of at least 1 percent, particularly in a range of 29 to 146-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a DNA mismatch repair protein, or if the activity of the polypeptide At3g18524, preferably represented by SEQ ID NO. 22833, or a homolog or fragment thereof, or if the activity of a polypeptide encoded by a nucleic acid molecule comprising the nucleic acid SEQ ID NO. 22832, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 22832 or polypeptide SEQ ID NO. 22833, respectively, or a homolog or a fragment thereof, is increased or generated, or if the activity DNA mismatch repair protein is increased or generated in a non-human organism, like a microorganism or a plant cell, plant or part thereof, especially with non-targeted localization, For example, an increase of the tryptophan of at least 1 percent, particularly in a range of 41 to 74-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a protein kinase, or if the activity of the polypeptide At3g23000, preferably represented by SEQ ID NO. 1816, or a homolog or fragment thereof, or if the activity of a polypeptide encoded by a nucleic acid molecule comprising the nucleic acid SEQ ID NO. 1815, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 1815 or polypeptide SEQ ID NO. 1816, respectively, or a homolog or a fragment thereof, is increased or generated, or if the activity protein kinase is increased or generated in a non-human organism, like a microorganism or a plant cell, plant or part thereof, especially with non-targeted localization, For example, an increase of the tryptophan of at least 1 percent, particularly in a range of 105 to 239-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a eukaryotic translation initiation factor, or if the activity of the polypeptide At3g26400, preferably represented by SEQ ID NO. 68728, or a homolog or fragment thereof, or if the activity of a polypeptide encoded by a nucleic acid molecule comprising the nucleic acid SEQ ID NO. 68727, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 68727 or polypeptide SEQ ID NO. 68728, respectively, or a homolog or a fragment thereof, is increased or generated, or if the activity eukaryotic translation initiation factor is increased or generated in a non-human organism, like a microorganism or a plant cell, plant or part thereof, especially with non-targeted localization, For example, an increase of the tryptophan of at least 1 percent, particularly in a range of 30 to 77-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a glycosyl transferase, or if the activity of the polypeptide At3g27540, preferably represented by SEQ ID NO. 68778, or a homolog or fragment thereof, or if the activity of a polypeptide encoded by a nucleic acid molecule comprising the nucleic acid SEQ ID NO. 68777, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 68777 or polypeptide SEQ ID NO. 68778, respectively, or a homolog or a fragment thereof, is increased or generated, or if the activity glycosyl transferase is increased or generated in a non-human organism, like a microorganism or a plant cell, plant or part thereof, especially with non-targeted localization, For example, an increase of the tryptophan of at least 1 percent, particularly in a range of 35 to 148-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a auxin response factor, or if the activity of the polypeptide At3g61830, preferably represented by SEQ ID NO. 2368, or a homolog or fragment thereof, or if the activity of a polypeptide encoded by a nucleic acid molecule comprising the nucleic acid SEQ ID NO. 2367, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 2367 or polypeptide SEQ ID NO. 2368, respectively, or a homolog or a fragment thereof, is increased or generated, or if the activity auxin response factor is increased or generated in a non-human organism, like a microorganism or a plant cell, plant or part thereof, especially with non-targeted localization, For example, an increase of the tryptophan of at least 1 percent, particularly in a range of 45 to 123-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a monthiol glutaredoxin, or if the activity of the polypeptide At3g62930, preferably represented by SEQ ID NO. 2574, or a homolog or fragment thereof, or if the activity of a polypeptide encoded by a nucleic acid molecule comprising the nucleic acid SEQ ID NO. 2573, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 2573 or polypeptide SEQ ID NO. 2574, respectively, or a homolog or a fragment thereof, is increased or generated, or if the activity monthiol glutaredoxin is increased or generated in a non-human organism, like a microorganism or a plant cell, plant or part thereof, especially with non-targeted localization, For example, an increase of the tryptophan of at least 1 percent, particularly in a range of 38 to 123-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, For example, an increase of the tryptophan of at least 1 percent, particularly in a range of 29 to 86-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a At4g32480protein, or if the activity of the polypeptide At4g32480, preferably represented by SEQ ID NO. 4041, or a homolog or fragment thereof, or if the activity of a polypeptide encoded by a nucleic acid molecule comprising the nucleic acid SEQ ID NO. 4040, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 4040 or polypeptide SEQ ID NO. 4041, respectively, or a homolog or a fragment thereof, is increased or generated, or if the activity At4g32480-protein is increased or generated in a non-human organism, like a microorganism or a plant cell, plant or part thereof, especially with non-targeted localization, For example, an increase of the tryptophan of at least 1 percent, particularly in a range of 41 to 460-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a cyclin D , or if the activity of the polypeptide At4g34160, preferably represented by SEQ ID NO. 24312, or a homolog or fragment thereof, or if the activity of a polypeptide encoded by a nucleic acid molecule comprising the nucleic acid SEQ ID NO. 24311, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 24311 or polypeptide SEQ ID NO. 24312, respectively, or a homolog or a fragment thereof, is increased or generated, or if the activity cyclin D is increased or generated in a non-human organism, like a microorganism or a plant cell, plant or part thereof, especially with non-targeted localization, For example, an increase of the tryptophan of at least 1 percent, particularly in a range of 36 to 123-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization, For example, an increase of the tryptophan of at least 1 percent, particularly in a range of 62 to 231-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a protein phosphatase, or if the activity of the polypeptide At5g02760, preferably represented by SEQ ID NO. 68988, or a homolog or fragment thereof, or if the activity of a polypeptide encoded by a nucleic acid molecule comprising the nucleic acid SEQ ID NO. 68987, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 68987 or polypeptide SEQ ID NO. 68988, respectively, or a homolog or a fragment thereof, is increased or generated, or if the activity protein phosphatase is increased or generated in a non-human organism, like a microorganism or a plant cell, plant or part thereof, especially with non-targeted localization, For example, an increase of the tryptophan of at least 1 percent, particularly in a range of 30 to 228-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a gibberellin 20-oxidase, or if the activity of the polypeptide At5g07200, preferably represented by SEQ ID NO. 24493, or a homolog or fragment thereof, or if the activity of a polypeptide encoded by a nucleic acid molecule comprising the nucleic acid SEQ ID NO. 24492, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 24492 or polypeptide SEQ ID NO. 24493, respectively, or a homolog or a fragment thereof, is increased or generated, or if the activity gibberellin 20-oxidase is increased or generated in a non-human organism, like a microorganism or a plant cell, plant or part thereof, especially with non-targeted localization, For example, an increase of the tryptophan of at least 1 percent, particularly in a range of 33 to 57-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a At5g16650protein, or if the activity of the polypeptide At5g16650, preferably represented by SEQ ID NO. 25284, or a homolog or fragment thereof, or if the activity of a polypeptide encoded by a nucleic acid molecule comprising the nucleic acid SEQ ID NO. 25283, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 25283 or polypeptide SEQ ID NO. 25284, respectively, or a homolog or a fragment thereof, is increased or generated, or if the activity At5g16650-protein is increased or generated in a non-human organism, like a microorganism or a plant cell, plant or part thereof, especially with non-targeted localization, For example, an increase of the tryptophan of at least 1 percent, particularly in a range of 32 to 83-percent is conferred as compared to a corresponding non-transformed wild type non-human organism

Accordingly, in one embodiment, an increase of tryptophan in a non-human organism, as compared to a corresponding non-transformed wild type non-human organism, is conferred in the process of the invention, if the activity of a polypeptide showing the activity of a at5g21910protein, or if the activity of the polypeptide At5g21910, preferably represented by SEQ ID NO. 69554, or a homolog or fragment thereof, or if the activity of a polypeptide encoded by a nucleic acid molecule comprising the nucleic acid SEQ ID NO. 69553, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 69553 or polypeptide SEQ ID NO. 69554, respectively, or a homolog or a fragment thereof, is increased or generated, or if the activity at5g21910-protein is increased or generated in a non-human organism, like a microorganism or a plant cell, plant or part thereof, especially with non-targeted localization, For example, an increase of the tryptophan of at least 1 percent, particularly in a range of 36 to 57-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a transcription factor, or if the activity of the polypeptide At5g39760, preferably represented by SEQ ID NO. 25429, or a homolog or fragment thereof, or if the activity of a polypeptide encoded by a nucleic acid molecule comprising the nucleic acid SEQ ID NO. 25428, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 25428 or polypeptide SEQ ID NO. 25429, respectively, or a homolog or a fragment thereof, is increased or generated, or if the activity transcription factor is increased or generated in a non-human organism, like a microorganism or a plant cell, plant or part thereof, especially with non-targeted localization, For example, an increase of the tryptophan of at least 1 percent, particularly in a range of 28 to 121-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a RNA-binding protein, or if the activity of the polypeptide At5g60110, preferably represented by SEQ ID NO. 70007, or a homolog or fragment thereof, or if the activity of a polypeptide encoded by a nucleic acid molecule comprising the nucleic acid SEQ ID NO. 70006, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 70006 or polypeptide SEQ ID NO. 70007, respectively, or a homolog or a fragment thereof, is increased or generated, or if the activity RNA-binding protein is increased or generated in a non-human organism, like a microorganism or a plant cell, plant or part thereof, especially with non-targeted localization, For example, an increase of the tryptophan of at least 1 percent, particularly in a range of 43 to 60-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a malic enzyme, or if the activity of the polypeptide AvinDRAFT_{—}1495, preferably represented by SEQ ID NO. 5558, or a homolog or fragment thereof, or if the activity of a polypeptide encoded by a nucleic acid molecule comprising the nucleic acid SEQ ID NO. 5557, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 5557 or polypeptide SEQ ID NO. 5558, respectively, or a homolog or a fragment thereof, is increased or generated, or if the activity malic enzyme is increased or generated in a non-human organism, like a microorganism or a plant cell, plant or part thereof, especially with non-targeted localization, For example, an increase of the tryptophan of at least 1 percent, particularly in a range of 62 to 203-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a elongation facfor Tu, or if the activity of the polypeptide AvinDRAFT_{—}2344, preferably represented by SEQ ID NO. 27022, or a homolog or fragment thereof, or if the activity of a polypeptide encoded by a nucleic acid molecule comprising the nucleic acid SEQ ID NO. 27021, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 27021 or polypeptide SEQ ID NO. 27022, respectively, or a homolog or a fragment thereof, is increased or generated, or if the activity elongation factor Tu is increased or generated in a non-human organism, like a microorganism or a plant cell, plant or part thereof, especially with non-targeted localization, For example, an increase of the tryptophan of at least 1 percent, particularly in a range of 34 to 173-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a enoyl-CoA hydratase, or if the activity of the polypeptide AvinDRAFT_{—}3159, preferably represented by SEQ ID NO. 28739, or a homolog or fragment thereof, or if the activity of a polypeptide encoded by a nucleic acid molecule comprising the nucleic acid SEQ ID NO. 28738, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 28738 or polypeptide SEQ ID NO. 28739, respectively, or a homolog or a fragment thereof, is increased or generated, or if the activity enoyl-CoA hydratase is increased or generated in a non-human organism, like a microorganism or a plant cell, plant or part thereof, especially with non-targeted localization, For example, an increase of the tryptophan of at least 1 percent, particularly in a range of 46 to 139-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a transcriptional regulator, or if the activity of the polypeptide AvinDRAFT_{—}3209, preferably represented by SEQ ID NO. 29287, or a homolog or fragment thereof, or if the activity of a polypeptide encoded by a nucleic acid molecule comprising the nucleic acid SEQ ID NO. 29286, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 29286 or polypeptide SEQ ID NO. 29287, respectively, or a homolog or a fragment thereof, is increased or generated, or if the activity transcriptional regulator is increased or generated in a non-human organism, like a microorganism or a plant cell, plant or part thereof, especially with non-targeted localization, For example, an increase of the tryptophan of at least 1 percent, particularly in a range of 34 to 143-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a enoyl-CoA hydratase, or if the activity of the polypeptide AvinDRAFT_{—}4420, preferably represented by SEQ ID NO. 73039, or a homolog or fragment thereof, or if the activity of a polypeptide encoded by a nucleic acid molecule comprising the nucleic acid SEQ ID NO. 73038, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 73038 or polypeptide SEQ ID NO. 73039, respectively, or a homolog or a fragment thereof, is increased or generated, or if the activity enoyl-CoA hydratase is increased or generated in a non-human organism, like a microorganism or a plant cell, plant or part thereof, especially with non-targeted localization, For example, an increase of the tryptophan of at least 1 percent, particularly in a range of 29 to 81-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a glutamateammonia -ligase, or if the activity of the polypeptide AvinDRAFT_{—}4836, preferably represented by SEQ ID NO. 74164, or a homolog or fragment thereof, or if the activity of a polypeptide encoded by a nucleic acid molecule comprising the nucleic acid SEQ ID NO. 74163, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 74163 or polypeptide SEQ ID NO. 74164, respectively, or a homolog or a fragment thereof, is increased or generated, or if the activity glutamate-ammonia-ligase is increased or generated in a non-human organism, like a microorganism or a plant cell, plant or part thereof, especially with non-targeted localization, For example, an increase of the tryptophan of at least 1 percent, particularly in a range of 35 to 163-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a hydrolase, or if the activity of the polypeptide AvinDRAFT_{—}5103, preferably represented by SEQ ID NO. 6511, or a homolog or fragment thereof, or if the activity of a polypeptide encoded by a nucleic acid molecule comprising the nucleic acid SEQ ID NO. 6510, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 6510 or polypeptide SEQ ID NO. 6511, respectively, or a homolog or a fragment thereof, is increased or generated, or if the activity hydrolase is increased or generated in a non-human organism, like a microorganism or a plant cell, plant or part thereof, especially with non-targeted localization, For example, an increase of the tryptophan of at least 1 percent, particularly in a range of 41 to 75-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a Chaperone protein CIpB, or if the activity of the polypeptide AvinDRAFT_{—}6093, preferably represented by SEQ ID NO. 33597, or a homolog or fragment thereof, or if the activity of a polypeptide encoded by a nucleic acid molecule comprising the nucleic acid SEQ ID NO. 33596, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 33596 or polypeptide SEQ ID NO. 33597, respectively, or a homolog or a fragment thereof, is increased or generated, or if the activity Chaperone protein CIpB is increased or generated in a non-human organism, like a microorganism or a plant cell, plant or part thereof, especially with non-targeted localization, For example, an increase of the tryptophan of at least 1 percent, particularly in a range of 59 to 124-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a electron transfer flavoprotein subunit beta, or if the activity of the polypeptide AvinDRAFT_{—}6679, preferably represented by SEQ ID NO. 74730, or a homolog or fragment thereof, or if the activity of a polypeptide encoded by a nucleic acid molecule comprising the nucleic acid SEQ ID NO. 74729, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 74729 or polypeptide SEQ ID NO. 74730, respectively, or a homolog or a fragment thereof, is increased or generated, or if the activity electron transfer flavoprotein subunit beta is increased or generated in a non-human organism, like a microorganism or a plant cell, plant or part thereof, especially with non-targeted localization, For example, an increase of the tryptophan of at least 1 percent, particularly in a range of 39 to 126-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a threonine synthase, or if the activity of the polypeptide B0004, preferably represented by SEQ ID NO. 34890, or a homolog or fragment thereof, or if the activity of a polypeptide encoded by a nucleic acid molecule comprising the nucleic acid SEQ ID NO. 34889, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 34889 or polypeptide SEQ ID NO. 34890, respectively, or a homolog or a fragment thereof, is increased or generated, or if the activity threonine synthase is increased or generated in a non-human organism, like a microorganism or a plant cell, plant or part thereof, especially with plastidic localization, For example, an increase of the tryptophan of at least 1 percent, particularly in a range of 57 to 168-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a transaldolase, or if the activity of the polypeptide B0008, preferably represented by SEQ ID NO. 98554, or a homolog or fragment thereof, or if the activity of a polypeptide encoded by a nucleic acid molecule comprising the nucleic acid SEQ ID NO. 98553, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 98553 or polypeptide SEQ ID NO. 98554, respectively, or a homolog or a fragment thereof, is increased or generated, or if the activity transaldolase is increased or generated in a non-human organism, like a microorganism or a plant cell, plant or part thereof, especially with non-targeted localization, For example, an increase of the tryptophan of at least 1 percent, particularly in a range of 51 to 91-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a L-ribulose-5-phosphate 4-epimerase, or if the activity of the polypeptide B0061, preferably represented by SEQ ID NO. 35205, or a homolog or fragment thereof, or if the activity of a polypeptide encoded by a nucleic acid molecule comprising the nucleic acid SEQ ID NO. 35204, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 35204 or polypeptide SEQ ID NO. 35205, respectively, or a homolog or a fragment thereof, is increased or generated, or if the activity L-ribulose-5-phosphate 4-epimerase is increased or generated in a non-human organism, like a microorganism or a plant cell, plant or part thereof, especially with non-targeted localization, For example, an increase of the tryptophan of at least 1 percent, particularly in a range of 72 to 173-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a glucose dehydrogenase, or if the activity of the polypeptide B0124, preferably represented by SEQ ID NO. 35483, or a homolog or fragment thereof, or if the activity of a polypeptide encoded by a nucleic acid molecule comprising the nucleic acid SEQ ID NO. 35482, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 35482 or polypeptide SEQ ID NO. 35483, respectively, or a homolog or a fragment thereof, is increased or generated, or if the activity glucose dehydrogenase is increased or generated in a non-human organism, like a microorganism or a plant cell, plant or part thereof, especially with non-targeted localization, For example, an increase of the tryptophan of at least 1 percent, particularly in a range of 29 to 213 -percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a ATP-binding component of a transport system, or if the activity of the polypeptide B0449, preferably represented by SEQ ID NO. 7334, or a homolog or fragment thereof, or if the activity of a polypeptide encoded by a nucleic acid molecule comprising the nucleic acid SEQ ID NO. 7333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 7333 or polypeptide SEQ ID NO. 7334, respectively, or a homolog or a fragment thereof, is increased or generated, or if the activity ATP-binding component of a transport system is increased or generated in a non-human organism, like a microorganism or a plant cell, plant or part thereof, especially with non-targeted localization, For example, an increase of the tryptophan of at least 1 percent, partitularly in a range of 38 to 281-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a isochorismate synthase, or if the activity of the polypeptide B0593, preferably represented by SEQ ID NO.

35968, or a homolog or fragment thereof, or if the activity of a polypeptide encoded by a nucleic acid molecule comprising the nucleic acid SEQ ID NO. 35967, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 35967 or polypeptide SEQ ID NO. 35968, respectively, or a homolog or a fragment thereof, is increased or generated, or if the activity isochorismate synthase is increased or generated in a non-human organism, like a microorganism or a plant cell, plant or part thereof, especially with plastidic localization, For example, an increase of the tryptophan of at least 1 percent, particularly in a range of 43 to 358 percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a Leucyl/phenylalanyl-tRNA-protein transferase, or if the activity of the polypeptide B0885, preferably represented by SEQ ID NO. 36490, or a homolog or fragment thereof, or if the activity of a polypeptide encoded by a nucleic acid molecule comprising the nucleic acid SEQ ID NO. 36489, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 36489 or polypeptide SEQ ID NO. 36490, respectively, or a homolog or a fragment thereof, is increased or generated, or if the activity Leucyl/phenylalanyl-tRNA-protein transferase is increased or generated in a non-human organism, like a microorganism or a plant cell, plant or part thereof, especially with non-targeted localization, For example, an increase of the tryptophan of at least 1 percent, particularly in a range of 39 to 143-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a b1163-protein, or if the activity of the polypeptide B1163, preferably represented by SEQ ID NO. 37395, or a homolog or fragment thereof, or if the activity of a polypeptide encoded by a nucleic acid molecule comprising the nucleic acid SEQ ID NO. 37394, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 37394 or polypeptide SEQ ID NO. 37395, respectively, or a homolog or a fragment thereof, is increased or generated, or if the activity b1163-protein is increased or generated in a non-human organism, like a microorganism or a plant cell, plant or part thereof, especially with non-targeted localization, For example, an increase of the tryptophan of at least 1 percent, particularly in a range of 30 to 56-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a cardiolipin synthase, or if the activity of the polypeptide B1249, preferably represented by SEQ ID NO. 98779, or a homolog or fragment thereof, or if the activity of a polypeptide encoded by a nucleic acid molecule comprising the nucleic acid SEQ ID NO. 98778, preferably the coding region thereof, or a homolog or fragment thereof, e.g. derived from Escherichia coli, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 98778 or polypeptide SEQ ID NO. 98779, respectively, or a homolog or a fragment thereof, is increased or generated, or if the activity cardiolipin synthase is increased or generated in a non-human organism, like a microorganism or a plant cell, plant or part thereof, especially with plastidic localization, For example, an increase of the tryptophan of at least 1 percent, particularly in a range of 71 to 214-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a tryptophan biosynthesis protein, or if the activity of the polypeptide B1262, preferably represented by SEQ ID NO. 76158, or a homolog or fragment thereof, or if the activity of a polypeptide encoded by a nucleic acid molecule comprising the nucleic acid SEQ ID NO. 76157, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 76157 or polypeptide SEQ ID NO. 76158, respectively, or a homolog or a fragment thereof, is increased or generated, or if the activity tryptophan biosynthesis protein is increased or generated in a non-human organism, like a microorganism or a plant cell, plant or part thereof, especially with plastidic localization, For example, an increase of the tryptophan of at least 1 percent, particularly in a range of 74 to 104-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a aldehyde dehydrogenase, or if the activity of the polypeptide B1300, preferably represented by SEQ ID NO.

37808, or a homolog or fragment thereof, or if the activity of a polypeptide encoded by a nucleic acid molecule comprising the nucleic acid SEQ ID NO. 37807, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 37807 or polypeptide SEQ ID NO. 37808, respectively, or a homolog or a fragment thereof, is increased or generated, or if the activity aldehyde dehydrogenase is increased or generated in a non-human organism, like a microorganism or a plant cell, plant or part thereof, especially with non-targeted localization, For example, an increase of the tryptophan of at least 1 percent, particularly in a range of 31 to 68-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a b1346-protein, or if the activity of the polypeptide B1346, preferably represented by SEQ ID NO. 99189, or a homolog or fragment thereof, or if the activity of a polypeptide encoded by a nucleic acid molecule comprising the nucleic acid SEQ ID NO. 99188, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 99188 or polypeptide SEQ ID NO. 99189, respectively, or a homolog or a fragment thereof, is increased or generated, or if the activity b1346-protein is increased or generated in a non-human organism, like a microorganism or a plant cell, plant or part thereof, especially with plastidic localization, For example, an increase of the tryptophan of at least 1 percent, particularly in a range of 31 to 96-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a b1522-protein, or if the activity of the polypeptide B1522, preferably represented by SEQ ID NO. 7948, or a homolog or fragment thereof, or if the activity of a polypeptide encoded by a nucleic acid molecule comprising the nucleic acid SEQ ID NO. 7947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 7947 or polypeptide SEQ ID NO. 7948, respectively, or a homolog or a fragment thereof, is increased or generated, or if the activity b1522-protein is increased or generated in a non-human organism, like a microorganism or a plant cell, plant or part thereof, especially with non-targeted localization, For example, an increase of the tryptophan of at least 1 percent, particularly in a range of 109 to 261-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a b1796-protein, or if the activity of the polypeptide B1796, preferably represented by SEQ ID NO. 99197, or a homolog or fragment thereof, or if the activity of a polypeptide encoded by a nucleic acid molecule comprising the nucleic acid SEQ ID NO. 99196, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 99196 or polypeptide SEQ ID NO. 99197, respectively, or a homolog or a fragment thereof, is increased or generated, or if the activity b1796-protein is increased or generated in a non-human organism, like a microorganism or a plant cell, plant or part thereof, especially with non-targeted localization, For example, an increase of the tryptophan of at least 1 percent, particularly in a range of 27 to 39-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a methylaccepting chemotaxis protein, or if the activity of the polypeptide B1886, preferably represented by SEQ ID NO. 99211, or a homolog or fragment thereof, or if the activity of a polypeptide encoded by a nucleic acid molecule comprising the nucleic acid SEQ ID NO. 99210, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 99210 or polypeptide SEQ ID NO. 99211, respectively, or a homolog or a fragment thereof, is increased or generated, or if the activity methyl-accepting chemotaxis protein is increased or generated in a non-human organism, like a microorganism or a plant cell, plant or part thereof, especially with non-targeted localization, For example, an increase of the tryptophan of at least 1 percent, particularly in a range of 54 to 108-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a trehalosephosphatase, or if the activity of the polypeptide B1897, preferably represented by SEQ ID NO. 79057, or a homolog or fragment thereof, or if the activity of a polypeptide encoded by a nucleic acid molecule comprising the nucleic acid SEQ ID NO. 79056, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 79056 or polypeptide SEQ ID NO. 79057, respectively, or a homolog or a fragment thereof, is increased or generated, or if the activity trehalose-phosphatase is increased or generated in a non-human organism, like a microorganism or a plant cell, plant or part thereof, especially with non-targeted localization, For example, an increase of the tryptophan of at least 1 percent, particularly in a range of 44 to 190-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a imidazole glycerol phosphate synthase subunit, or if the activity of the polypeptide B2023, preferably represented by SEQ ID NO. 99372, or a homolog or fragment thereof, or if the activity of a polypeptide encoded by a nucleic acid molecule comprising the nucleic acid SEQ ID NO. 99371, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 99371 or polypeptide SEQ ID NO. 99372, respectively, or a homolog or a fragment thereof, is increased or generated, or if the activity imidazole glycerol phosphate synthase subunit is increased or generated in a non-human organism, like a microorganism or a plant cell, plant or part thereof, especially with non-targeted localization, For example, an increase of the tryptophan of at least 1 percent, particularly in a range of 44 to 148-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a transport protein, or if the activity of the polypeptide B2063, preferably represented by SEQ ID NO. 38948, or a homolog or fragment thereof, or if the activity of a polypeptide encoded by a nucleic acid molecule comprising the nucleic acid SEQ ID NO. 38947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 38947 or polypeptide SEQ ID NO. 38948, respectively, or a homolog or a fragment thereof, is increased or generated, or if the activity transport protein is increased or generated in a non-human organism, like a microorganism or a plant cell, plant or part thereof, especially with non-targeted localization, For example, an increase of the tryptophan of at least 1 percent, particularly in a range of 35 to 270-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a ATP synthase subunit E, or if the activity of the polypeptide B2285, preferably represented by SEQ ID NO. 99621, or a homolog or fragment thereof, or if the activity of a polypeptide encoded by a nucleic acid molecule comprising the nucleic acid SEQ ID NO. 99620, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 99620 or polypeptide SEQ ID NO. 99621, respectively, or a homolog or a fragment thereof, is increased or generated, or if the activity ATP synthase subunit E is increased or generated in a non-human organism, like a microorganism or a plant cell, plant or part thereof, especially with non-targeted localization, For example, an increase of the tryptophan of at least 1 percent, particularly in a range of 57 to 109-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a putative transport system permease protein, or if the activity of the polypeptide B2546, preferably represented by SEQ ID NO. 99899, or a homolog or fragment thereof, or if the activity of a polypeptide encoded by a nucleic acid molecule comprising the nucleic acid SEQ ID NO. 99898, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 99898 or polypeptide SEQ ID NO. 99899, respectively, or a homolog or a fragment thereof, is increased or generated, or if the activity putative transport system permease protein is increased or generated in a non-human organism, like a microorganism or a plant cell, plant or part thereof, especially with non-targeted localization, For example, an increase of the tryptophan of at least 1 percent, particularly in a range of 32 to 95-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a recombinase A, or if the activity of the polypeptide B2699, preferably represented by SEQ ID NO. 100106, or a homolog or fragment thereof, or if the activity of a polypeptide encoded by a nucleic acid molecule comprising the nucleic acid SEQ ID NO. 100105, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 100105 or polypeptide SEQ ID NO. 100106, respectively, or a homolog or a fragment thereof, is increased or generated, or if the activity recombinase A is increased or generated in a non-human organism, like a microorganism or a plant cell, plant or part thereof, especially with non-targeted localization, For example, an increase of the tryptophan of at least 1 percent, particularly in a range of 55 to 82-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a b2739-protein, or if the activity of the polypeptide B2739, preferably represented by SEQ ID NO. 80907, or a homolog or fragment thereof, or if the activity of a polypeptide encoded by a nucleic acid molecule comprising the nucleic acid SEQ ID NO. 80906, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 80906 or polypeptide SEQ ID NO. 80907, respectively, or a homolog or a fragment thereof, is increased or generated, or if the activity b2739-protein is increased or generated in a non-human organism, like a microorganism or a plant cell, plant or part thereof, especially with non-targeted localization, For example, an increase of the tryptophan of at least 1 percent, particularly in a range of 75 to 134-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a b2849-protein, or if the activity of the polypeptide B2849, preferably represented by SEQ ID NO. 81981, or a homolog or fragment thereof, or if the activity of a polypeptide encoded by a nucleic acid molecule comprising the nucleic acid SEQ ID NO. 81980, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 81980 or polypeptide SEQ ID NO. 81981, respectively, or a homolog or a fragment thereof, is increased or generated, or if the activity b2849-protein is increased or generated in a non-human organism, like a microorganism or a plant cell, plant or part thereof, especially with non-targeted localization, For example, an increase of the tryptophan of at least 1 percent, particularly in a range of 33 to 152-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a fimbrial-like protein, or if the activity of the polypeptide B3142, preferably represented by SEQ ID NO. 100544, or a homolog or fragment thereof, or if the activity of a polypeptide encoded by a nucleic acid molecule comprising the nucleic acid SEQ ID NO. 100543, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 100543 or polypeptide SEQ ID NO. 100544, respectively, or a homolog or a fragment thereof, is increased or generated, or if the activity fimbrial-like protein is increased or generated in a non-human organism, like a microorganism or a plant cell, plant or part thereof, especially with non-targeted localization, For example, an increase of the tryptophan of at least 1 percent, particularly in a range of 35 to 65-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a methyltransferase, or if the activity of the polypeptide B3262, preferably represented by SEQ ID NO. 42503, or a homolog or fragment thereof, or if the activity of a polypeptide encoded by a nucleic acid molecule comprising the nucleic acid SEQ ID NO. 42502, preferably the coding region thereof, or a homolog or fragment thereof, e.g. derived from Escherichia coli, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 42502 or polypeptide SEQ ID NO. 42503, respectively, or a homolog or a fragment thereof, is increased or generated, or if the activity methyltransferase is increased or generated in a non-human organism, like a microorganism or a plant cell, plant or part thereof, especially with non-targeted localization, For example, an increase of the tryptophan of at least 1 percent, particularly in a range of 47 to 277-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a 3-ketoacyl-CoA thiolase, or if the activity of the polypeptide B3845, preferably represented by SEQ ID NO. 100676, or a homolog or fragment thereof, or if the activity of a polypeptide encoded by a nucleic acid molecule comprising the nucleic acid SEQ ID NO. 100675, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 100675 or polypeptide SEQ ID NO. 100676, respectively, or a homolog or a fragment thereof, is increased or generated, or if the activity 3-ketoacyl-CoA thiolase is increased or generated in a non-human organism, like a microorganism or a plant cell, plant or part thereof, especially with plastidic localization, For example, an increase of the tryptophan of at least 1 percent, particularly in a range of 56 to 87-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a glutamine synthetase, or if the activity of the polypeptide B3870, preferably represented by SEQ ID NO. 101328, or a homolog or fragment thereof, or if the activity of a polypeptide encoded by a nucleic acid molecule comprising the nucleic acid SEQ ID NO. 101327, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 101327 or polypeptide SEQ ID NO. 101328, respectively, or a homolog or a fragment thereof, is increased or generated, or if the activity glutamine synthetase is increased or generated in a non-human organism, like a microorganism or a plant cell, plant or part thereof, especially with plastidic localization, For example, an increase of the tryptophan of at least 1 percent, particularly in a range of 47 to 85-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a b3989-protein, or if the activity of the polypeptide B3989, preferably represented by SEQ ID NO. 44373, or a homolog or fragment thereof, or if the activity of a polypeptide encoded by a nucleic acid molecule comprising the nucleic acid SEQ ID NO. 44372, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 44372 or polypeptide SEQ ID NO. 44373, respectively, or a homolog or a fragment thereof, is increased or generated, or if the activity b3989-protein is increased or generated in a non-human organism, like a microorganism or a plant cell, plant or part thereof, especially with non-targeted localization, For example, an increase of the tryptophan of at least 1 percent, particularly in a range of 32 to 398-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a b4029-protein, or if the activity of the polypeptide B4029, preferably represented by SEQ ID NO. 10741, or a homolog or fragment thereof, or if the activity of a polypeptide encoded by a nucleic acid molecule comprising the nucleic acid SEQ ID NO. 10740, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 10740 or polypeptide SEQ ID NO. 10741, respectively, or a homolog or a fragment thereof, is increased or generated, or if the activity b4029-protein is increased or generated in a non-human organism, like a microorganism or a plant cell, plant or part thereof, especially with non-targeted localization, For example, an increase of the tryptophan of at least 1 percent, particularly in a range of 27 to 814-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a b4121-protein, or if the activity of the polypeptide B4121, preferably represented by SEQ ID NO. 44610, or a homolog or fragment thereof, or if the activity of a polypeptide encoded by a nucleic acid molecule comprising the nucleic acid SEQ ID NO. 44609, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 44609 or polypeptide SEQ ID NO. 44610, respectively, or a homolog or a fragment thereof, is increased or generated, or if the activity b4121-protein is increased or generated in a non-human organism, like a microorganism or a plant cell, plant or part thereof, especially with non-targeted localization, For example, an increase of the tryptophan of at least 1 percent, particularly in a range of 30 to 46-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a acetyltransferase, or if the activity of the polypeptide B4256, preferably represented by SEQ ID NO. 45322, or a homolog or fragment thereof, or if the activity of a polypeptide encoded by a nucleic acid molecule comprising the nucleic acid SEQ ID NO. 45321, preferably the coding region thereof, or a homolog or fragment thereof, e.g. derived from Escherichia coli, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 45321 or polypeptide SEQ ID NO. 45322, respectively, or a homolog or a fragment thereof, is increased or generated, or if the activity acetyltransferase is increased or generated in a non-human organism, like a microorganism or a plant cell, plant or part thereof, especially with non-targeted localization, For example, an increase of the tryptophan of at least 1 percent, particularly in a range of 34 to 217-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a RNA binding protein, or if the activity of the polypeptide GM02LC17556, preferably represented by SEQ ID NO. 46516, or a homolog or fragment thereof, or if the activity of a polypeptide encoded by a nucleic acid molecule comprising the nucleic acid SEQ ID NO. 46515, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 46515 or polypeptide SEQ ID NO. 46516, respectively, or a homolog or a fragment thereof, is increased or generated, or if the activity RNA binding protein is increased or generated in a non-human organism, like a microorganism or a plant cell, plant or part thereof, especially with non-targeted localization, For example, an increase of the tryptophan of at least 1 percent, particularly in a range of 37 to 149-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a transcription factor, or if the activity of the polypeptide GM02LC21368, preferably represented by SEQ ID NO. 84199, or a homolog or fragment thereof, or if the activity of a polypeptide encoded by a nucleic acid molecule comprising the nucleic acid SEQ ID NO. 84198, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 84198 or polypeptide SEQ ID NO. 84199, respectively, or a homolog or a fragment thereof, is increased or generated, or if the activity transcription factor is increased or generated in a non-human organism, like a microorganism or a plant cell, plant or part thereof, especially with non-targeted localization, For example, an increase of the tryptophan of at least 1 percent, particularly in a range of 43 to 224-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a choline kinase, or if the activity of the polypeptide S_pp020029328r, preferably represented by SEQ ID NO. 101781, or a homolog or fragment thereof, or if the activity of a polypeptide encoded by a nucleic acid molecule comprising the nucleic acid SEQ ID NO. 101780, preferably the coding region thereof, or a homolog or fragment thereof, e.g. derived from Physcomitrella patens, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 101780 or polypeptide SEQ ID NO. 101781, respectively, or a homolog or a fragment thereof, is increased or generated, or if the activity choline kinase is increased or generated in a non-human organism, like a microorganism or a plant cell, plant or part thereof, especially with non-targeted localization, For example, an increase of the tryptophan of at least 1 percent, particularly in a range of 40 to 55-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a flavodoxin, or if the activity of the polypeptide Sll0248, preferably represented by SEQ ID NO. 49144, or a homolog or fragment thereof, or if the activity of a polypeptide encoded by a nucleic acid molecule comprising the nucleic acid SEQ ID NO. 49143, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 49143 or polypeptide SEQ ID NO. 49144, respectively, or a homolog or a fragment thereof, is increased or generated, or if the activity flavodoxin is increased or generated in a non-human organism, like a microorganism or a plant cell, plant or part thereof, especially with plastidic localization, For example, an increase of the tryptophan of at least 1 percent, particularly in a range of 32 to 169-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a NAD(P)H-quinone oxidoreductase subunit, or if the activity of the polypeptide 5110521, preferably represented by SEQ ID NO. 50340, or a homolog or fragment thereof, or if the activity of a polypeptide encoded by a nucleic acid molecule comprising the nucleic acid SEQ ID NO. 50339, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 50339 or polypeptide SEQ ID NO. 50340, respectively, or a homolog or a fragment thereof, is increased or generated, or if the activity NAD(P)H-quinone oxidoreductase subunit is increased or generated in a non-human organism, like a microorganism or a plant cell, plant or part thereof, especially with plastidic localization, For example, an increase of the tryptophan of at least 1 percent, particularly in a range of 31 to 49-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a binding-protein-dependent transport systems inner membrane component, or if the activity of the polypeptide Sll0833, preferably represented by SEQ ID NO. 101708, or a homolog or fragment thereof, or if the activity of a polypeptide encoded by a nucleic acid molecule comprising the nucleic acid SEQ ID NO. 101707, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 101707 or polypeptide SEQ ID NO. 101708, respectively, or a homolog or a fragment thereof, is increased or generated, or if the activity binding-protein-dependent transport systems inner membrane component is increased or generated in a non-human organism, like a microorganism or a plant cell, plant or part thereof, especially with non-targeted localization, For example, an increase of the tryptophan of at least 1 percent, particularly in a range of 42 to 92-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a carbon dioxide concentrating mechanism protein, or if the activity of the polypeptide Sll1031, preferably represented by SEQ ID NO. 52635, or a homolog or fragment thereof, or if the activity of a polypeptide encoded by a nucleic acid molecule comprising the nucleic acid SEQ ID NO. 52634, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 52634 or polypeptide SEQ ID NO. 52635, respectively, or a homolog or a fragment thereof, is increased or generated, or if the activity carbon dioxide concentrating mechanism protein is increased or generated in a non-human organism, like a microorganism or a plant cell, plant or part thereof, especially with mitochondrial localization, For example, an increase of the tryptophan of at least 1 percent, particularly in a range of 28 to 167-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a nitrate/nitrite transport protein, or if the activity of the polypeptide Sll1450, preferably represented by SEQ ID NO. 54338, or a homolog or fragment thereof, or if the activity of a polypeptide encoded by a nucleic acid molecule comprising the nucleic acid SEQ ID NO. 54337, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 54337 or polypeptide SEQ ID NO. 54338, respectively, or a homolog or a fragment thereof, is increased or generated, or if the activity nitrate/nitrite transport protein is increased or generated in a non-human organism, like a microorganism or a plant cell, plant or part thereof, especially with mitochondrial localization, For example, an increase of the tryptophan of at least 1 percent, particularly in a range of 42 to 85-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a coproporphyrinogen III oxidase, or if the activity of the polypeptide Sll1917, preferably represented by SEQ ID NO. 11472, or a homolog or fragment thereof, or if the activity of a polypeptide encoded by a nucleic acid molecule comprising the nucleic acid SEQ ID NO. 11471, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 11471 or polypeptide SEQ ID NO. 11472, respectively, or a homolog or a fragment thereof, is increased or generated, or if the activity coproporphyrinogen III oxidase is increased or generated in a non-human organism, like a microorganism or a plant cell, plant or part thereof, especially with mitochondrial localization, For example, an increase of the tryptophan of at least 1 percent, particularly in a range of 31 to 36-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a precorrin methylase, or if the activity of the polypeptide Slr0239, preferably represented by SEQ ID NO. 86541, or a homolog or fragment thereof, or if the activity of a polypeptide encoded by a nucleic acid molecule comprising the nucleic acid SEQ ID NO. 86540, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 86540 or polypeptide SEQ ID NO. 86541, respectively, or a homolog or a fragment thereof, is increased or generated, or if the activity precorrin methylase is increased or generated in a non-human organism, like a microorganism or a plant cell, plant or part thereof, especially with mitochondrial localization, For example, an increase of the tryptophan of at least 1 percent, particularly in a range of 37 to 162-percent is conferred as compared to a corresponding non-transformed wild type non-human organism

Accordingly, in one embodiment, an increase of tryptophan in a non-human organism, as compared to a corresponding non-transformed wild type non-human organism, is conferred in the process of the invention, if the activity of a polypeptide showing the activity of a malate dehydrogenase, or if the activity of the polypeptide Slr0721, preferably represented by SEQ ID NO. 87398, or a homolog or fragment thereof, or if the activity of a polypeptide encoded by a nucleic acid molecule comprising the nucleic acid SEQ ID NO. 87397, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 87397 or polypeptide SEQ ID NO. 87398, respectively, or a homolog or a fragment thereof, is increased or generated, or if the activity malate dehydrogenase is increased or generated in a non-human organism, like a microorganism or a plant cell, plant or part thereof, especially with plastidic localization, For example, an increase of the tryptophan of at least 1 percent, particularly in a range of 46 to 100-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a geranylgeranyl pyrophosphate synthase, or if the activity of the polypeptide Slr0739, preferably represented by SEQ ID NO. 57236, or a homolog or fragment thereof, or if the activity of a polypeptide encoded by a nucleic acid molecule comprising the nucleic acid SEQ ID NO. 57235, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 57235 or polypeptide SEQ ID NO. 57236, respectively, or a homolog or a fragment thereof, is increased or generated, or if the activity geranylgeranyl pyrophosphate synthase is increased or generated in a non-human organism, like a microorganism or a plant cell, plant or part thereof, especially with plastidic localization, For example, an increase of the tryptophan of at least 1 percent, particularly in a range of 35 to 54-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a slr1107-protein, or if the activity of the polypeptide Slr1107, preferably represented by SEQ ID NO. 101777, or a homolog or fragment thereof, or if the activity of a polypeptide encoded by a nucleic acid molecule comprising the nucleic acid SEQ ID NO. 101776, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 101776 or polypeptide SEQ ID NO. 101777, respectively, or a homolog or a fragment thereof, is increased or generated, or if the activity slr1107-protein is increased or generated in a non-human organism, like a microorganism or a plant cell, plant or part thereof, especially with plastidic localization, For example, an increase of the tryptophan of at least 1 percent, particularly in a range of 30 to 54-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a photosystem II protein, or if the activity of the polypeptide Slr1739, preferably represented by SEQ ID NO. 58732, or a homolog or fragment thereof, or if the activity of a polypeptide encoded by a nucleic acid molecule comprising the nucleic acid SEQ ID NO. 58731, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 58731 or polypeptide SEQ ID NO. 58732, respectively, or a homolog or a fragment thereof, is increased or generated, or if the activity photosystem II protein is increased or generated in a non-human organism, like a microorganism or a plant cell, plant or part thereof, especially with plastidic localization, For example, an increase of the tryptophan of at least 1 percent, particularly in a range of 86 to 157-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a short-chain alcohol dehydrogenase family, or if the activity of the polypeptide Slr2124, preferably represented by SEQ ID NO. 59371, or a homolog or fragment thereof, or if the activity of a polypeptide encoded by a nucleic acid molecule comprising the nucleic acid SEQ ID NO. 59370, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 59370 or polypeptide SEQ ID NO. 59371, respectively, or a homolog or a fragment thereof, is increased or generated, or if the activity short-chain alcohol dehydrogenase family is increased or generated in a non-human organism, like a microorganism or a plant cell, plant or part thereof, especially with plastidic localization, For example, an increase of the tryptophan of at least 1 percent, particularly in a range of 38 to 80-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a Sec-independent protein translocase subunit, or if the activity of the polypeptide TTC0019, preferably represented by SEQ ID NO. 12699, or a homolog or fragment thereof, or if the activity of a polypeptide encoded by a nucleic acid molecule comprising the nucleic acid SEQ ID NO. 12698, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 12698 or polypeptide SEQ ID NO. 12699, respectively, or a homolog or a fragment thereof, is increased or generated, or if the activity Sec-independent protein translocase subunit is increased or generated in a non-human organism, like a microorganism or a plant cell, plant or part thereof, especially with non-targeted localization, For example, an increase of the tryptophan of at least 1 percent, particularly in a range of 306 to 472-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a 3-deoxy-7-phosphoheptulonate synthase, or if the activity of the polypeptide Ybr249c, preferably represented by SEQ ID NO. 88880, or a homolog or fragment thereof, or if the activity of a polypeptide encoded by a nucleic acid molecule comprising the nucleic acid SEQ ID NO. 88879, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 88879 or polypeptide SEQ ID NO. 88880, respectively, or a homolog or a fragment thereof, is increased or generated, or if the activity 3-deoxy-7-phosphoheptulonate synthase is increased or generated in a non-human organism, like a microorganism or a plant cell, plant or part thereof, especially with plastidic localization, For example, an increase of the tryptophan of at least 1 percent, particularly in a range of 59 to 405-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a branched-chain amino acid permease, or if the activity of the polypeptide Ydr046c, preferably represented by SEQ ID NO. 63545, or a homolog or fragment thereof, or if the activity of a polypeptide encoded by a nucleic acid molecule comprising the nucleic acid SEQ ID NO. 63544, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 63544 or polypeptide SEQ ID NO. 63545, respectively, or a homolog or a fragment thereof, is increased or generated, or if the activity branched-chain amino acid permease is increased or generated in a non-human organism, like a microorganism or a plant cell, plant or part thereof, especially with plastidic localization, For example, an increase of the tryptophan of at least 1 percent, particularly in a range of 30 to 63-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a yfl054c-protein, or if the activity of the polypeptide Yfl054c, preferably represented by SEQ ID NO. 64149, or a homolog or fragment thereof, or if the activity of a polypeptide encoded by a nucleic acid molecule comprising the nucleic acid SEQ ID NO. 64148, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 64148 or polypeptide SEQ ID NO. 64149, respectively, or a homolog or a fragment thereof, is increased or generated, or if the activity yfl054c-protein is increased or generated in a non-human organism, like a microorganism or a plant cell, plant or part thereof, especially with plastidic localization, For example, an increase of the tryptophan of at least 1 percent, particularly in a range of 30 to 41-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a glycerol kinase, or if the activity of the polypeptide Yhl032c, preferably represented by SEQ ID NO. 101856, or a homolog or fragment thereof, or if the activity of a polypeptide encoded by a nucleic acid molecule comprising the nucleic acid SEQ ID NO. 101855, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 101855 or polypeptide SEQ ID NO. 101856, respectively, or a homolog or a fragment thereof, is increased or generated, or if the activity glycerol kinase is increased or generated in a non-human organism, like a microorganism or a plant cell, plant or part thereof, especially with plastidic localization, For example, an increase of the tryptophan of at least 1 percent, particularly in a range of 61 to 101-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a aromatic amino acid aminotransferase, or if the activity of the polypeptide Yhr137w, preferably represented by SEQ ID NO. 102016, or a homolog or fragment thereof, or if the activity of a polypeptide encoded by a nucleic acid molecule comprising the nucleic acid SEQ ID NO. 102015, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 102015 or polypeptide SEQ ID NO. 102016, respectively, or a homolog or a fragment thereof, is increased or generated, or if the activity aromatic amino acid aminotransferase is increased or generated in a non-human organism, like a microorganism or a plant cell, plant or part thereof, especially with plastidic localization, For example, an increase of the tryptophan of at least 1 percent, particularly in a range of 29 to 77-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a cystathionine gamma-synthase, or if the activity of the polypeptide Yjr130c, preferably represented by SEQ ID NO. 14844, or a homolog or fragment thereof, or if the activity of a polypeptide encoded by a nucleic acid molecule comprising the nucleic acid SEQ ID NO. 14843, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 14843 or polypeptide SEQ ID NO. 14844, respectively, or a homolog or a fragment thereof, is increased or generated, or if the activity cystathionine gamma-synthase is increased or generated in a non-human organism, like a microorganism or a plant cell, plant or part thereof, especially with non-targeted localization, For example, an increase of the tryptophan of at least 1 percent, particularly in a range of 30 to 62-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a polygalacturonase, or if the activity of the polypeptide Yjr153w, preferably represented by SEQ ID NO. 66275, or a homolog or fragment thereof, or if the activity of a polypeptide encoded by a nucleic acid molecule comprising the nucleic acid SEQ ID NO. 66274, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 66274 or polypeptide SEQ ID NO. 66275, respectively, or a homolog or a fragment thereof, is increased or generated, or if the activity polygalacturonase is increased or generated in a non-human organism, like a microorganism or a plant cell, plant or part thereof, especially with non-targeted localization, For example, an increase of the tryptophan of at least 1 percent, particularly in a range of 34 to 115-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a lipase, or if the activity of the polypeptide Ykr089c, preferably represented by SEQ ID NO. 102062, or a homolog or fragment thereof, or if the activity of a polypeptide encoded by a nucleic acid molecule comprising the nucleic acid SEQ ID NO. 102061, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 102061 or polypeptide SEQ ID NO. 102062, respectively, or a homolog or a fragment thereof, is increased or generated, or if the activity lipase is increased or generated in a non-human organism, like a microorganism or a plant cell, plant or part thereof, especially with mitochondrial localization, For example, an increase of the tryptophan of at least 1 percent, particularly in a range of 60 to 288-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a amino acid transporter, or if the activity of the polypeptide Ynl101w, preferably represented by SEQ ID NO. 92340, or a homolog or fragment thereof, or if the activity of a polypeptide encoded by a nucleic acid molecule comprising the nucleic acid SEQ ID NO. 92339, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 92339 or polypeptide SEQ ID NO. 92340, respectively, or a homolog or a fragment thereof, is increased or generated, or if the activity amino acid transporter is increased or generated in a non-human organism, like a microorganism or a plant cell, plant or part thereof, especially with plastidic localization, For example, an increase of the tryptophan of at least 1 percent, particularly in a range of 33 to 45 percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a histone acetyltransferase, or if the activity of the polypeptide Yor244w, preferably represented by SEQ ID NO. 92541, or a homolog or fragment thereof, or if the activity of a polypeptide encoded by a nucleic acid molecule comprising the nucleic acid SEQ ID NO. 92540, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 92540 or polypeptide SEQ ID NO. 92541, respectively, or a homolog or a fragment thereof, is increased or generated, or if the activity histone acetyltransferase is increased or generated in a non-human organism, like a microorganism or a plant cell, plant or part thereof, especially with plastidic localization, For example, an increase of the tryptophan of at least 1 percent, particularly in a range of 39 to 135-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a isopentenyl diphosphate isomerase, or if the activity of the polypeptide Ypl117c, preferably represented by SEQ ID NO. 67999, or a homolog or fragment thereof, or if the activity of a polypeptide encoded by a nucleic acid molecule comprising the nucleic acid SEQ ID NO. 67998, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 67998 or polypeptide SEQ ID NO. 67999, respectively, or a homolog or a fragment thereof, is increased or generated, or if the activity isopentenyl diphosphate isomerase is increased or generated in a non-human organism, like a microorganism or a plant cell, plant or part thereof, especially with plastidic localization, For example, an increase of the tryptophan of at least 1 percent, particularly in a range of 29 to 59-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a cyclin, or if the activity of the polypeptide Ypr119w, preferably represented by SEQ ID NO. 98446, or a homolog or fragment thereof, or if the activity of a polypeptide encoded by a nucleic acid molecule comprising the nucleic acid SEQ ID NO. 98445, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 98445 or polypeptide SEQ ID NO. 98446, respectively, or a homolog or a fragment thereof, is increased or generated, or if the activity cyclin is increased or generated in a non-human organism, like a microorganism or a plant cell, plant or part thereof, especially with non-targeted localization, For example, an increase of the tryptophan of at least 1 percent, particularly in a range of 36 to 54-percent is conferred as compared to a corresponding non-transformed wild type non-human organism.

Accordingly, in one embodiment, an increase of tryptophan in a non-human organism, as compared to a corresponding non-transformed wild type non-human organism, is conferred in the process of the invention, if the activity of a polypeptide showing the activity of a Zm_{—}4842_BE510522-protein, or if the activity of the polypeptide Zm_{—}4842_BE510522, preferably represented by SEQ ID NO. 68414, or a homolog or fragment thereof, or if the activity of a polypeptide encoded by a nucleic acid molecule comprising the nucleic acid SEQ ID NO. 68413, preferably the coding region thereof, or a homolog or fragment thereof, e.g. derived from Zea mays, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 5, column 5 or 8 in the respective same line as the nucleic acid molecule SEQ ID NO. 68413 or polypeptide SEQ ID NO. 68414, respectively, or a homolog or a fragment thereof, is increased or generated, or if the activity Zm_{—}4842_BE510522-protein is increased or generated in a non-human organism, like a microorganism or a plant cell, plant or part thereof, especially with non-targeted localization, For example, an increase of the tryptophan of at least 1 percent, particularly in a range of 53 to 117-percent is conferred as compared to a corresponding non-transformed wild type non-human organism.

[0096.1.1.5] to [0103.1.1.5] for the disclosure of these paragraphs see [0096.1.1.1] to [0103.1.1.1] above.

In this paragraph “A” means “Goffeau et al., Science 274 (5287), 546 (1996)”, “B” means “Blattner et al., Science 277 (5331), 1453 (1997)”, “C” means “Nakamura et al., Nucleic Acids Res. 28(1), 72 (2000)”, “D” means “deposition of the sequences to the EMBL/GenBank/DDBJ databases under “Sequencing of the draft genome assembly of Azotobacter vinelandii AvOP”. in June 2005 by Copeland et al.”, “E” means “Henne et al., Nat. Biotechnol. 22 (5), 547 (2004)”, “F” means “Kaul et al., Nature 408 (6814), 796 (2000)”, and “G” means “Matsumoto et al., Nature 436 (7052), 793 (2005)”.

The nucleic acid sequence of 59582753_SOYBEAN from Glycine max, e.g. as shown in the respective line in column 5 of Table I, application no. 5, is unpublished. And the activity of the gene product thereof is the activity of glucose-6-phosphate 1-dehydrogenase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “glucose-6-phosphate 1-dehydrogenase”, especially from Glycine max or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said 59582753_SOYBEAN, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said 59582753_SOYBEAN, and preferably the activity is increased plastidic, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said 59582753_SOYBEAN, or a functional equivalent or a homolog thereof as depicted in column 8 of Table II, application no. 5, preferably a homolog or functional equivalent as depicted in column 8 of Table II B, application no. 5, and being depicted in the same respective line as said 59582753_SOYBEAN, and preferably the activity is increased plastidic.

Accordingly, in one embodiment, the molecule which activity is to be increased in the process of the invention is the gene product with an activity as a “glucose-6-phosphate 1-dehydrogenase”, preferably it is the molecule of section (a) or (b) of this paragraph.

In particular, it was observed that in plants, especially in Arabidopsis thaliana, increasing or generating the activity of a gene product plastidic with the activity of a “glucose-6-phosphate 1-dehydrogenase”, preferably being encoded by a gene comprising the nucleic acid sequence SEQ ID NO. 16883, preferably the coding region thereof, conferred the production of or the increase in tryptophan compared with the wild type control.

The nucleic acid sequence of At1g19800 from Arabidopsis thaliana, e.g. as shown in the respective line in column 5 of Table I, application no. 5, has been published in F. And the activity of the gene product thereof is the activity of At1g19800-protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “At1g19800-protein”, especially from Arabidopsis thaliana or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said At1g19800, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said At1g19800, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said At1g19800, or a functional equivalent or a homolog thereof as depicted in column 8 of Table II, application no. 5, preferably a homolog or functional equivalent as depicted in column 8 of Table II B, application no. 5, and being depicted in the same respective line as said At1g19800, and preferably the activity is increased non-targeted.

Accordingly, in one embodiment, the molecule which activity is to be increased in the process of the invention is the gene product with an activity as a “At1g19800-protein”, preferably it is the molecule of section (a) or (b) of this paragraph.

In particular, it was observed that in plants, especially in Arabidopsis thaliana, increasing or generating the activity of a gene product non-targeted with the activity of a “At1g19800-protein”, preferably being encoded by a gene comprising the nucleic acid sequence SEQ ID NO. 17637, preferably the coding region thereof, conferred the production of or the increase in tryptophan compared with the wild type control.

The nucleic acid sequence of At1g26830 from Arabidopsis thaliana, e.g. as shown in the respective line in column 5 of Table I, application no. 5, has been published in F. And the activity of the gene product thereof is the activity of cullin.

Accordingly, in one embodiment, the process of the present invention for producing tryptophan in a non-human organism, like a microorganism or a plant or a part thereof, comprises increasing or generating the activity of a gene product with the activity of a gene product conferring the activity of “cullin”, especially from Arabidopsis thaliana or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said At1g26830, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said At1g26830, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said At1g26830, or a functional equivalent or a homolog thereof as depicted in column 8 of Table II, application no. 5, preferably a homolog or functional equivalent as depicted in column 8 of Table II B, application no. 5, and being depicted in the same respective line as said At1g26830, and preferably the activity is increased non-targeted.

Accordingly, in one embodiment, the molecule which activity is to be increased in the process of the invention is the gene product with an activity as a “cullin”, preferably it is the molecule of section (a) or (b) of this paragraph.

In particular, it was observed that in plants, especially in Arabidopsis thaliana, increasing or generating the activity of a gene product non-targeted with the activity of a “cullin”, preferably being encoded by a gene comprising the nucleic acid sequence SEQ ID NO. 17701, preferably the coding region thereof, conferred the production of or the increase in tryptophan compared with the wild type control.

The nucleic acid sequence of At1g36730 from Arabidopsis thaliana, e.g. as shown in the respective line in column 5 of Table I, application no. 5, has been published in F. And the activity of the gene product thereof is the activity of eukaryotic translation initiation factor 5.

Accordingly, in one embodiment, the process of the present invention for producing tryptophan in a non-human organism, like a microorganism or a plant or a part thereof, comprises increasing or generating the activity of a gene product with the activity of a gene product conferring the activity of “eukaryotic translation initiation factor 5”, especially from Arabidopsis thaliana or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said At1g36730, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said At1g36730, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said At1g36730, or a functional equivalent or a homolog thereof as depicted in column 8 of Table II, application no. 5, preferably a homolog or functional equivalent as depicted in column 8 of Table II B, application no. 5, and being depicted in the same respective line as said At1g36730, and preferably the activity is increased non-targeted.

Accordingly, in one embodiment, the molecule which activity is to be increased in the process of the invention is the gene product with an activity as a “eukaryotic translation initiation factor 5”, preferably it is the molecule of section (a) or (b) of this paragraph.

In particular, it was observed that in plants, especially in Arabidopsis thaliana, increasing or generating the activity of a gene product non-targeted with the activity of a “eukaryotic translation initiation factor 5”, preferably being encoded by a gene comprising the nucleic acid sequence SEQ ID NO. 17968, preferably the coding region thereof, conferred the production of or the increase in tryptophan compared with the wild type control.

The nucleic acid sequence of At1g48260 from Arabidopsis thaliana, e.g. as shown in the respective line in column 5 of Table I, application no. 5, has been published in F. And the activity of the gene product thereof is the activity of protein kinase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said At1g48260, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said At1g48260, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said At1g48260, or a functional equivalent or a homolog thereof as depicted in column 8 of Table II, application no. 5, preferably a homolog or functional equivalent as depicted in column 8 of Table II B, application no. 5, and being depicted in the same respective line as said At1g48260, and preferably the activity is increased non-targeted.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 18235, preferably the coding region thereof, conferred the production of or the increase in tryptophan compared with the wild type control.

The nucleic acid sequence of At2g17560 from Arabidopsis thaliana, e.g. as shown in the respective line in column 5 of Table I, application no. 5, has been published in F. And the activity of the gene product thereof is the activity of transcription factor.

Accordingly, in one embodiment, the process of the present invention for producing tryptophan in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said At2g17560, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said At2g17560, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said At2g17560, or a functional equivalent or a homolog thereof as depicted in column 8 of Table II, application no. 5, preferably a homolog or functional equivalent as depicted in column 8 of Table II B, application no. 5, and being depicted in the same respective line as said At2g17560, and preferably the activity is increased non-targeted.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 19502, preferably the coding region thereof, conferred the production of or the increase in tryptophan compared with the wild type control.

The nucleic acid sequence of At2g28890 from Arabidopsis thaliana, e.g. as shown in the respective line in column 5 of Table I, application no. 5, has been published in F. And the activity of the gene product thereof is the activity of protein phosphatase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said At2g28890, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said At2g28890, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said At2g28890, or a functional equivalent or a homolog thereof as depicted in column 8 of Table II, application no. 5, preferably a homolog or functional equivalent as depicted in column 8 of Table II B, application no. 5, and being depicted in the same respective line as said At2g28890, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19874, preferably the coding region thereof, conferred the production of or the increase in tryptophan compared with the wild type control.

The nucleic acid sequence of At2g30360 from Arabidopsis thaliana, e.g. as shown in the respective line in column 5 of Table I, application no. 5, has been published in F. And the activity of the gene product thereof is the activity of CBL-interacting protein kinase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “CBL-interacting protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said At2g30360, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said At2g30360, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said At2g30360, or a functional equivalent or a homolog thereof as depicted in column 8 of Table II, application no. 5, preferably a homolog or functional equivalent as depicted in column 8 of Table II B, application no. 5, and being depicted in the same respective line as said At2g30360, and preferably the activity is increased non-targeted.

Accordingly, in one embodiment, the molecule which activity is to be increased in the process of the invention is the gene product with an activity as a “CBL-interacting protein kinase”, preferably it is the molecule of section (a) or (b) of this paragraph.

In particular, it was observed that in plants, especially in Arabidopsis thaliana, increasing or generating the activity of a gene product non-targeted with the activity of a “CBL-interacting protein kinase”, preferably being encoded by a gene comprising the nucleic acid sequence SEQ ID NO. 19919, preferably the coding region thereof, conferred the production of or the increase in tryptophan compared with the wild type control.

The nucleic acid sequence of At2g30540 from Arabidopsis thaliana, e.g. as shown in the respective line in column 5 of Table I, application no. 5, has been published in F. And the activity of the gene product thereof is the activity of glutaredoxin.

Accordingly, in one embodiment, the process of the present invention for producing tryptophan in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said At2g30540, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said At2g30540, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said At2g30540, or a functional equivalent or a homolog thereof as depicted in column 8 of Table II, application no. 5, preferably a homolog or functional equivalent as depicted in column 8 of Table II B, application no. 5, and being depicted in the same respective line as said At2g30540, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 20346, preferably the coding region thereof, conferred the production of or the increase in tryptophan compared with the wild type control.

The nucleic acid sequence of At2g33510 from Arabidopsis thaliana, e.g. as shown in the respective line in column 5 of Table I, application no. 5, has been published in F. And the activity of the gene product thereof is the activity of At2g33510-protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “At2g33510-protein”, especially from Arabidopsis thaliana or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said At2g33510, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said At2g33510, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said At2g33510, or a functional equivalent or a homolog thereof as depicted in column 8 of Table II, application no. 5, preferably a homolog or functional equivalent as depicted in column 8 of Table II B, application no. 5, and being depicted in the same respective line as said At2g33510, and preferably the activity is increased non-targeted.

Accordingly, in one embodiment, the molecule which activity is to be increased in the process of the invention is the gene product with an activity as a “At2g33510-protein”, preferably it is the molecule of section (a) or (b) of this paragraph.

In particular, it was observed that in plants, especially in Arabidopsis thaliana, increasing or generating the activity of a gene product non-targeted with the activity of a “At2g33510-protein”, preferably being encoded by a gene comprising the nucleic acid sequence SEQ ID NO. 98522, preferably the coding region thereof, conferred the production of or the increase in tryptophan compared with the wild type control.

The nucleic acid sequence of At2g47880 from Arabidopsis thaliana, e.g. as shown in the respective line in column 5 of Table I, application no. 5, has been published in F. And the activity of the gene product thereof is the activity of glutaredoxin.

Accordingly, in one embodiment, the process of the present invention for producing tryptophan in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said At2g47880, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said At2g47880, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said At2g47880, or a functional equivalent or a homolog thereof as depicted in column 8 of Table II, application no. 5, preferably a homolog or functional equivalent as depicted in column 8 of Table II B, application no. 5, and being depicted in the same respective line as said At2g47880, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 21159, preferably the coding region thereof, conferred the production of or the increase in tryptophan compared with the wild type control. The nucleic acid sequence of At3g04050 from Arabidopsis thaliana, e.g. as shown in the respective line in column 5 of Table I, application no. 5, has been published in F. And the activity of the gene product thereof is the activity of pyruvate kinase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “pyruvate kinase”, especially from Arabidopsis thaliana or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said At3g04050, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said At3g04050, and preferably the activity is increased plastidic, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said At3g04050, or a functional equivalent or a homolog thereof as depicted in column 8 of Table II, application no. 5, preferably a homolog or functional equivalent as depicted in column 8 of Table II B, application no. 5, and being depicted in the same respective line as said At3g04050, and preferably the activity is increased plastidic.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 21497, preferably the coding region thereof, conferred the production of or the increase in tryptophan compared with the wild type control.

The nucleic acid sequence of At3g11650 from Arabidopsis thaliana, e.g. as shown in the respective line in column 5 of Table I, application no. 5, has been published in F. And the activity of the gene product thereof is the activity of harpin-induced family protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “harpin-induced family protein”, especially from Arabidopsis thaliana or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said At3g11650, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said At3g11650, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said At3g11650, or a functional equivalent or a homolog thereof as depicted in column 8 of Table II, application no. 5, preferably a homolog or functional equivalent as depicted in column 8 of Table II B, application no. 5, and being depicted in the same respective line as said At3g11650, and preferably the activity is increased non-targeted.

Accordingly, in one embodiment, the molecule which activity is to be increased in the process of the invention is the gene product with an activity as a “harpin-induced family protein”, preferably it is the molecule of section (a) or (b) of this paragraph.

In particular, it was observed that in plants, especially in Arabidopsis thaliana, increasing or generating the activity of a gene product non-targeted with the activity of a “harpin-induced family protein”, preferably being encoded by a gene comprising the nucleic acid sequence SEQ ID NO. 22611, preferably the coding region thereof, conferred the production of or the increase in tryptophan compared with the wild type control.

The nucleic acid sequence of At3g18524 from Arabidopsis thaliana, e.g. as shown in the respective line in column 5 of Table I, application no. 5, has been published in F. And the activity of the gene product thereof is the activity of DNA mismatch repair protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “DNA mismatch repair protein”, especially from Arabidopsis thaliana or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said At3g18524, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said At3g18524, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said At3g18524, or a functional equivalent or a homolog thereof as depicted in column 8 of Table II, application no. 5, preferably a homolog or functional equivalent as depicted in column 8 of Table II B, application no. 5, and being depicted in the same respective line as said At3g18524, and preferably the activity is increased non-targeted.

Accordingly, in one embodiment, the molecule which activity is to be increased in the process of the invention is the gene product with an activity as a “DNA mismatch repair protein”, preferably it is the molecule of section (a) or (b) of this paragraph.

In particular, it was observed that in plants, especially in Arabidopsis thaliana, increasing or generating the activity of a gene product non-targeted with the activity of a “DNA mismatch repair protein”, preferably being encoded by a gene comprising the nucleic acid sequence SEQ ID NO. 22832, preferably the coding region thereof, conferred the production of or the increase in tryptophan compared with the wild type control.

The nucleic acid sequence of At3g23000 from Arabidopsis thaliana, e.g. as shown in the respective line in column 5 of Table I, application no. 5, has been published in F. And the activity of the gene product thereof is the activity of protein kinase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said At3g23000, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said At3g23000, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said At3g23000, or a functional equivalent or a homolog thereof as depicted in column 8 of Table II, application no. 5, preferably a homolog or functional equivalent as depicted in column 8 of Table II B, application no. 5, and being depicted in the same respective line as said At3g23000, and preferably the activity is increased non-targeted.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 1815, preferably the coding region thereof, conferred the production of or the increase in tryptophan compared with the wild type control.

The nucleic acid sequence of At3g26400 from Arabidopsis thaliana, e.g. as shown in the respective line in column 5 of Table I, application no. 5, has been published in F. And the activity of the gene product thereof is the activity of eukaryotic translation initiation factor.

Accordingly, in one embodiment, the process of the present invention for producing tryptophan in a non-human organism, like a microorganism or a plant or a part thereof, comprises increasing or generating the activity of a gene product with the activity of a gene product conferring the activity of “eukaryotic translation initiation factor”, especially from Arabidopsis thaliana or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said At3g26400, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said At3g26400, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said At3g26400, or a functional equivalent or a homolog thereof as depicted in column 8 of Table II, application no. 5, preferably a homolog or functional equivalent as depicted in column 8 of Table II B, application no. 5, and being depicted in the same respective line as said At3g26400, and preferably the activity is increased non-targeted.

Accordingly, in one embodiment, the molecule which activity is to be increased in the process of the invention is the gene product with an activity as a “eukaryotic translation initiation factor”, preferably it is the molecule of section (a) or (b) of this paragraph.

In particular, it was observed that in plants, especially in Arabidopsis thaliana, increasing or generating the activity of a gene product non-targeted with the activity of a “eukaryotic translation initiation factor”, preferably being encoded by a gene comprising the nucleic acid sequence SEQ ID NO. 68727, preferably the coding region thereof, conferred the production of or the increase in tryptophan compared with the wild type control.

The nucleic acid sequence of At3g27540 from Arabidopsis thaliana, e.g. as shown in the respective line in column 5 of Table I, application no. 5, has been published in F. And the activity of the gene product thereof is the activity of glycosyl transferase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “glycosyl transferase”, especially from Arabidopsis thaliana or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said At3g27540, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said At3g27540, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said At3g27540, or a functional equivalent or a homolog thereof as depicted in column 8 of Table II, application no. 5, preferably a homolog or functional equivalent as depicted in column 8 of Table II B, application no. 5, and being depicted in the same respective line as said At3g27540, and preferably the activity is increased non-targeted.

Accordingly, in one embodiment, the molecule which activity is to be increased in the process of the invention is the gene product with an activity as a “glycosyl transferase”, preferably it is the molecule of section (a) or (b) of this paragraph.

In particular, it was observed that in plants, especially in Arabidopsis thaliana, increasing or generating the activity of a gene product non-targeted with the activity of a “glycosyl transferase”, preferably being encoded by a gene comprising the nucleic acid sequence SEQ ID NO. 68777, preferably the coding region thereof, conferred the production of or the increase in tryptophan compared with the wild type control.

The nucleic acid sequence of At3g61830 from Arabidopsis thaliana, e.g. as shown in the respective line in column 5 of Table I, application no. 5, has been published in F. And the activity of the gene product thereof is the activity of auxin response factor.

Accordingly, in one embodiment, the process of the present invention for producing tryptophan in a non-human organism, like a microorganism or a plant or a part thereof, comprises increasing or generating the activity of a gene product with the activity of a gene product conferring the activity of “auxin response factor”, especially from Arabidopsis thaliana or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said At3g61830, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said At3g61830, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said At3g61830, or a functional equivalent or a homolog thereof as depicted in column 8 of Table II, application no. 5, preferably a homolog or functional equivalent as depicted in column 8 of Table II B, application no. 5, and being depicted in the same respective line as said At3g61830, and preferably the activity is increased non-targeted.

Accordingly, in one embodiment, the molecule which activity is to be increased in the process of the invention is the gene product with an activity as a “auxin response factor”, preferably it is the molecule of section (a) or (b) of this paragraph.

In particular, it was observed that in plants, especially in Arabidopsis thaliana, increasing or generating the activity of a gene product non-targeted with the activity of a “auxin response factor”, preferably being encoded by a gene comprising the nucleic acid sequence SEQ ID NO. 2367, preferably the coding region thereof, conferred the production of or the increase in tryptophan compared with the wild type control.

The nucleic acid sequence of At3g62930 from Arabidopsis thaliana, e.g. as shown in the respective line in column 5 of Table I, application no. 5, has been published in F. And the activity of the gene product thereof is the activity of monthiol glutaredoxin.

Accordingly, in one embodiment, the process of the present invention for producing tryptophan in a non-human organism, like a microorganism or a plant or a part thereof, comprises increasing or generating the activity of a gene product with the activity of a gene product conferring the activity of “monthiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said At3g62930, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said At3g62930, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said At3g62930, or a functional equivalent or a homolog thereof as depicted in column 8 of Table II, application no. 5, preferably a homolog or functional equivalent as depicted in column 8 of Table II B, application no. 5, and being depicted in the same respective line as said At3g62930, and preferably the activity is increased non-targeted.

Accordingly, in one embodiment, the molecule which activity is to be increased in the process of the invention is the gene product with an activity as a “monthiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monthiol glutaredoxin”, preferably being encoded by a gene comprising the nucleic acid sequence SEQ ID NO.

2573, preferably the coding region thereof, conferred the production of or the increase in tryptophan compared with the wild type control.

The nucleic acid sequence of At3g62950 from Arabidopsis thaliana, e.g. as shown in the respective line in column 5 of Table I, application no. 5, has been published in F. And the activity of the gene product thereof is the activity of glutaredoxin.

Accordingly, in one embodiment, the process of the present invention for producing tryptophan in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said At3g62950, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said At3g62950, or a functional equivalent or a homolog thereof as depicted in column 8 of Table II, application no. 5, preferably a homolog or functional equivalent as depicted in column 8 of Table II B, application no. 5, and being depicted in the same respective line as said At3g62950, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in tryptophan compared with the wild type control.

The nucleic acid sequence of At4g32480 from Arabidopsis thaliana, e.g. as shown in the respective line in column 5 of Table I, application no. 5, has been published in F. And the activity of the gene product thereof is the activity of At4g32480-protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “At4g32480-protein”, especially from Arabidopsis thaliana or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said At4g32480, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said At4g32480, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said At4g32480, or a functional equivalent or a homolog thereof as depicted in column 8 of Table II, application no. 5, preferably a homolog or functional equivalent as depicted in column 8 of Table II B, application no. 5, and being depicted in the same respective line as said At4g32480, and preferably the activity is increased non-targeted.

Accordingly, in one embodiment, the molecule which activity is to be increased in the process of the invention is the gene product with an activity as a “At4g32480-protein”, preferably it is the molecule of section (a) or (b) of this paragraph.

In particular, it was observed that in plants, especially in Arabidopsis thaliana, increasing or generating the activity of a gene product non-targeted with the activity of a “At4g32480-protein”, preferably being encoded by a gene comprising the nucleic acid sequence SEQ ID NO. 4040, preferably the coding region thereof, conferred the production of or the increase in tryptophan compared with the wild type control.

The nucleic acid sequence of At4g34160 from Arabidopsis thaliana, e.g. as shown in the respective line in column 5 of Table I, application no. 5, has been published in F. And the activity of the gene product thereof is the activity of cyclin D .

Accordingly, in one embodiment, the process of the present invention for producing tryptophan in a non-human organism, like a microorganism or a plant or a part thereof, comprises increasing or generating the activity of a gene product with the activity of a gene product conferring the activity of “cyclin D”, especially from Arabidopsis thaliana or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said At4g34160, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said At4g34160, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said At4g34160, or a functional equivalent or a homolog thereof as depicted in column 8 of Table II, application no. 5, preferably a homolog or functional equivalent as depicted in column 8 of Table II B, application no. 5, and being depicted in the same respective line as said At4g34160, and preferably the activity is increased non-targeted.

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 24311, preferably the coding region thereof, conferred the production of or the increase in tryptophan compared with the wild type control.

The nucleic acid sequence of At4g35310 from Arabidopsis thaliana, e.g. as shown in the respective line in column 5 of Table I, application no. 5, has been published in F. And the activity of the gene product thereof is the activity of calcium-dependent protein kinase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said At4g35310, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said At4g35310, or a functional equivalent or a homolog thereof as depicted in column 8 of Table II, application no. 5, preferably a homolog or functional equivalent as depicted in column 8 of Table II B, application no. 5, and being depicted in the same respective line as said At4g35310, and preferably the activity is increased non-targeted.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in tryptophan compared with the wild type control.

The nucleic acid sequence of At5g02760 from Arabidopsis thaliana, e.g. as shown in the respective line in column 5 of Table I, application no. 5, has been published in F. And the activity of the gene product thereof is the activity of protein phosphatase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said At5g02760, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said At5g02760, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said At5g02760, or a functional equivalent or a homolog thereof as depicted in column 8 of Table II, application no. 5, preferably a homolog or functional equivalent as depicted in column 8 of Table II B, application no. 5, and being depicted in the same respective line as said At5g02760, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 68987, preferably the coding region thereof, conferred the production of or the increase in tryptophan compared with the wild type control.

The nucleic acid sequence of At5g07200 from Arabidopsis thaliana, e.g. as shown in the respective line in column 5 of Table I, application no. 5, has been published in F. And the activity of the gene product thereof is the activity of gibberellin 20-oxidase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “gibberellin 20-oxidase”, especially from Arabidopsis thaliana or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said At5g07200, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said At5g07200, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said At5g07200, or a functional equivalent or a homolog thereof as depicted in column 8 of Table II, application no. 5, preferably a homolog or functional equivalent as depicted in column 8 of Table II B, application no. 5, and being depicted in the same respective line as said At5g07200, and preferably the activity is increased non-targeted.

Accordingly, in one embodiment, the molecule which activity is to be increased in the process of the invention is the gene product with an activity as a “gibberellin 20-oxidase”, preferably it is the molecule of section (a) or (b) of this paragraph.

In particular, it was observed that in plants, especially in Arabidopsis thaliana, increasing or generating the activity of a gene product non-targeted with the activity of a “gibberellin 20-oxidase”, preferably being encoded by a gene comprising the nucleic acid sequence SEQ ID NO. 24492, preferably the coding region thereof, conferred the production of or the increase in tryptophan compared with the wild type control.

The nucleic acid sequence of At5g16650 from Arabidopsis thaliana, e.g. as shown in the respective line in column 5 of Table I, application no. 5, has been published in F. And the activity of the gene product thereof is the activity of At5g16650-protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “At5g16650-protein”, especially from Arabidopsis thaliana or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said At5g16650, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said At5g16650, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said At5g16650, or a functional equivalent or a homolog thereof as depicted in column 8 of Table II, application no. 5, preferably a homolog or functional equivalent as depicted in column 8 of Table II B, application no. 5, and being depicted in the same respective line as said At5g16650, and preferably the activity is increased non-targeted.

Accordingly, in one embodiment, the molecule which activity is to be increased in the process of the invention is the gene product with an activity as a “At5g16650-protein”, preferably it is the molecule of section (a) or (b) of this paragraph.

In particular, it was observed that in plants, especially in Arabidopsis thaliana, increasing or generating the activity of a gene product non-targeted with the activity of a “At5g16650-protein”, preferably being encoded by a gene comprising the nucleic acid sequence SEQ ID NO. 25283, preferably the coding region thereof, conferred the production of or the increase in tryptophan compared with the wild type control.

The nucleic acid sequence of At5g21910 from Arabidopsis thaliana, e.g. as shown in the respective line in column 5 of Table I, application no. 5, has been published in F. And the activity of the gene product thereof is the activity of at5g21910-protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “at5g21910-protein”, especially from Arabidopsis thaliana or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said At5g21910, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said At5g21910, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said At5g21910, or a functional equivalent or a homolog thereof as depicted in column 8 of Table II, application no. 5, preferably a homolog or functional equivalent as depicted in column 8 of Table II B, application no. 5, and being depicted in the same respective line as said At5g21910, and preferably the activity is increased non-targeted.

Accordingly, in one embodiment, the molecule which activity is to be increased in the process of the invention is the gene product with an activity as a “at5g21910-protein”, preferably it is the molecule of section (a) or (b) of this paragraph.

In particular, it was observed that in plants, especially in Arabidopsis thaliana, increasing or generating the activity of a gene product non-targeted with the activity of a “at5g21910-protein”, preferably being encoded by a gene comprising the nucleic acid sequence SEQ ID NO. 69553, preferably the coding region thereof, conferred the production of or the increase in tryptophan compared with the wild type control.

The nucleic acid sequence of At5g39760 from Arabidopsis thaliana, e.g. as shown in the respective line in column 5 of Table I, application no. 5, has been published in F. And the activity of the gene product thereof is the activity of transcription factor.

Accordingly, in one embodiment, the process of the present invention for producing tryptophan in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said At5g39760, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said At5g39760, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said At5g39760, or a functional equivalent or a homolog thereof as depicted in column 8 of Table II, application no. 5, preferably a homolog or functional equivalent as depicted in column 8 of Table II B, application no. 5, and being depicted in the same respective line as said At5g39760, and preferably the activity is increased non-targeted.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 25428, preferably the coding region thereof, conferred the production of or the increase in tryptophan compared with the wild type control.

The nucleic acid sequence of At5g60110 from Arabidopsis thaliana, e.g. as shown in the respective line in column 5 of Table I, application no. 5, has been published in F. And the activity of the gene product thereof is the activity of RNA-binding protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “RNA-binding protein”, especially from Arabidopsis thaliana or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said At5g60110, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said At5g60110, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said At5g60110, or a functional equivalent or a homolog thereof as depicted in column 8 of Table II, application no. 5, preferably a homolog or functional equivalent as depicted in column 8 of Table II B, application no. 5, and being depicted in the same respective line as said At5g60110, and preferably the activity is increased non-targeted.

Accordingly, in one embodiment, the molecule which activity is to be increased in the process of the invention is the gene product with an activity as a “RNA-binding protein”, preferably it is the molecule of section (a) or (b) of this paragraph.

In particular, it was observed that in plants, especially in Arabidopsis thaliana, increasing or generating the activity of a gene product non-targeted with the activity of a “RNA-binding protein”, preferably being encoded by a gene comprising the nucleic acid sequence SEQ ID NO. 70006, preferably the coding region thereof, conferred the production of or the increase in tryptophan compared with the wild type control.

The nucleic acid sequence of AvinDRAFT_{—}1495 from Azotobacter vinelandii, e.g. as shown in the respective line in column 5 of Table I, application no. 5, has been published in D. And the activity of the gene product thereof is the activity of malic enzyme.

Accordingly, in one embodiment, the process of the present invention for producing tryptophan in a non-human organism, like a microorganism or a plant or a part thereof, comprises increasing or generating the activity of a gene product with the activity of a gene product conferring the activity of “malic enzyme”, especially from Azotobacter vinelandii or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said AvinDRAFT_{—}1495, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said AvinDRAFT_{—}1495, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said AvinDRAFT_{—}1495, or a functional equivalent or a homolog thereof as depicted in column 8 of Table II, application no. 5, preferably a homolog or functional equivalent as depicted in column 8 of Table II B, application no. 5, and being depicted in the same respective line as said AvinDRAFT_{—}1495, and preferably the activity is increased non-targeted.

Accordingly, in one embodiment, the molecule which activity is to be increased in the process of the invention is the gene product with an activity as a “malic enzyme”, preferably it is the molecule of section (a) or (b) of this paragraph.

In particular, it was observed that in plants, especially in Arabidopsis thaliana, increasing or generating the activity of a gene product non-targeted with the activity of a “malic enzyme”, preferably being encoded by a gene comprising the nucleic acid sequence SEQ ID NO. 5557, preferably the coding region thereof, conferred the production of or the increase in tryptophan compared with the wild type control.

The nucleic acid sequence of AvinDRAFT_{—}2344 from Azotobacter vinelandii, e.g. as shown in the respective line in column 5 of Table I, application no. 5, has been published in D. And the activity of the gene product thereof is the activity of elongation factor Tu.

Accordingly, in one embodiment, the process of the present invention for producing tryptophan in a non-human organism, like a microorganism or a plant or a part thereof, comprises increasing or generating the activity of a gene product with the activity of a gene product conferring the activity of “elongation factor Tu”, especially from Azotobacter vinelandii or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said AvinDRAFT_{—}2344, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said AvinDRAFT_{—}2344, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said AvinDRAFT_{—}2344, or a functional equivalent or a homolog thereof as depicted in column 8 of Table II, application no. 5, preferably a homolog or functional equivalent as depicted in column 8 of Table II B, application no. 5, and being depicted in the same respective line as said AvinDRAFT_{—}2344, and preferably the activity is increased non-targeted.

Accordingly, in one embodiment, the molecule which activity is to be increased in the process of the invention is the gene product with an activity as a “elongation factor Tu”, preferably it is the molecule of section (a) or (b) of this paragraph.

In particular, it was observed that in plants, especially in Arabidopsis thaliana, increasing or generating the activity of a gene product non-targeted with the activity of a “elongation factor Tu”, preferably being encoded by a gene comprising the nucleic acid sequence SEQ ID NO. 27021, preferably the coding region thereof, conferred the production of or the increase in tryptophan compared with the wild type control.

The nucleic acid sequence of AvinDRAFT_{—}3159 from Azotobacter vinelandii, e.g. as shown in the respective line in column 5 of Table I, application no. 5, has been published in D. And the activity of the gene product thereof is the activity of enoyl-CoA hydratase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “enoyl-CoA hydratase”, especially from Azotobacter vinelandii or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said AvinDRAFT_{—}3159, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said AvinDRAFT_{—}3159, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said AvinDRAFT_{—}3159, or a functional equivalent or a homolog thereof as depicted in column 8 of Table II, application no. 5, preferably a homolog or functional equivalent as depicted in column 8 of Table II B, application no. 5, and being depicted in the same respective line as said AvinDRAFT_{—}3159, and preferably the activity is increased non-targeted.

Accordingly, in one embodiment, the molecule which activity is to be increased in the process of the invention is the gene product with an activity as a “enoyl-CoA hydratase”, preferably it is the molecule of section (a) or (b) of this paragraph.

In particular, it was observed that in plants, especially in Arabidopsis thaliana, increasing or generating the activity of a gene product non-targeted with the activity of a “enoyl-CoA hydratase”, preferably being encoded by a gene comprising the nucleic acid sequence SEQ ID NO. 28738, preferably the coding region thereof, conferred the production of or the increase in tryptophan compared with the wild type control.

The nucleic acid sequence of AvinDRAFT_{—}3209 from Azotobacter vinelandii, e.g. as shown in the respective line in column 5 of Table I, application no. 5, has been published in D. And the activity of the gene product thereof is the activity of transcriptional regulator.

Accordingly, in one embodiment, the process of the present invention for producing tryptophan in a non-human organism, like a microorganism or a plant or a part thereof, comprises increasing or generating the activity of a gene product with the activity of a gene product conferring the activity of “transcriptional regulator”, especially from Azotobacter vinelandii or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said AvinDRAFT_{—}3209, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said AvinDRAFT_{—}3209, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said AvinDRAFT_{—}3209, or a functional equivalent or a homolog thereof as depicted in column 8 of Table II, application no. 5, preferably a homolog or functional equivalent as depicted in column 8 of Table II B, application no. 5, and being depicted in the same respective line as said AvinDRAFT_{—}3209, and preferably the activity is increased non-targeted.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 29286, preferably the coding region thereof, conferred the production of or the increase in tryptophan compared with the wild type control.

The nucleic acid sequence of AvinDRAFT_{—}4420 from Azotobacter vinelandii, e.g. as shown in the respective line in column 5 of Table I, application no. 5, has been published in D. And the activity of the gene product thereof is the activity of enoyl-CoA hydratase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “enoyl-CoA hydratase”, especially from Azotobacter vinelandii or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said AvinDRAFT_{—}4420, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said AvinDRAFT_{—}4420, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said AvinDRAFT_{—}4420, or a functional equivalent or a homolog thereof as depicted in column 8 of Table II, application no. 5, preferably a homolog or functional equivalent as depicted in column 8 of Table II B, application no. 5, and being depicted in the same respective line as said AvinDRAFT_{—}4420, and preferably the activity is increased non-targeted.

Accordingly, in one embodiment, the molecule which activity is to be increased in the process of the invention is the gene product with an activity as a “enoyl-CoA hydratase”, preferably it is the molecule of section (a) or (b) of this paragraph.

In particular, it was observed that in plants, especially in Arabidopsis thaliana, increasing or generating the activity of a gene product non-targeted with the activity of a “enoyl-CoA hydratase”, preferably being encoded by a gene comprising the nucleic acid sequence SEQ ID NO. 73038, preferably the coding region thereof, conferred the production of or the increase in tryptophan compared with the wild type control.

The nucleic acid sequence of AvinDRAFT_{—}4836 from Azotobacter vinelandii, e.g. as shown in the respective line in column 5 of Table I, application no. 5, has been published in D. And the activity of the gene product thereof is the activity of glutamate-ammonia-ligase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “glutamate-ammonia-ligase”, especially from Azotobacter vinelandii or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said AvinDRAFT_{—}4836, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said AvinDRAFT_{—}4836, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said AvinDRAFT_{—}4836, or a functional equivalent or a homolog thereof as depicted in column 8 of Table II, application no. 5, preferably a homolog or functional equivalent as depicted in column 8 of Table II B, application no. 5, and being depicted in the same respective line as said AvinDRAFT_{—}4836, and preferably the activity is increased non-targeted.

Accordingly, in one embodiment, the molecule which activity is to be increased in the process of the invention is the gene product with an activity as a “glutamate-ammonia-ligase”, preferably it is the molecule of section (a) or (b) of this paragraph.

In particular, it was observed that in plants, especially in Arabidopsis thaliana, increasing or generating the activity of a gene product non-targeted with the activity of a “glutamate-ammonia-ligase”, preferably being encoded by a gene comprising the nucleic acid sequence SEQ ID NO. 74163, preferably the coding region thereof, conferred the production of or the increase in tryptophan compared with the wild type control.

The nucleic acid sequence of AvinDRAFT_{—}5103 from Azotobacter vinelandii, e.g. as shown in the respective line in column 5 of Table I, application no. 5, has been published in D. And the activity of the gene product thereof is the activity of hydrolase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “hydrolase”, especially from Azotobacter vinelandii or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said AvinDRAFT_{—}5103, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said AvinDRAFT_{—}5103, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said AvinDRAFT_{—}5103, or a functional equivalent or a homolog thereof as depicted in column 8 of Table II, application no. 5, preferably a homolog or functional equivalent as depicted in column 8 of Table II B, application no. 5, and being depicted in the same respective line as said AvinDRAFT_{—}5103, and preferably the activity is increased non-targeted.

Accordingly, in one embodiment, the molecule which activity is to be increased in the process of the invention is the gene product with an activity as a “hydrolase”, preferably it is the molecule of section (a) or (b) of this paragraph.

In particular, it was observed that in plants, especially in Arabidopsis thaliana, increasing or generating the activity of a gene product non-targeted with the activity of a “hydrolase”, preferably being encoded by a gene comprising the nucleic acid sequence SEQ ID NO. 6510, preferably the coding region thereof, conferred the production of or the increase in tryptophan compared with the wild type control.

The nucleic acid sequence of AvinDRAFT_{—}6093 from Azotobacter vinelandii, e.g. as shown in the respective line in column 5 of Table I, application no. 5, has been published in D. And the activity of the gene product thereof is the activity of Chaperone protein CIpB.

Accordingly, in one embodiment, the process of the present invention for producing tryptophan in a non-human organism, like a microorganism or a plant or a part thereof, comprises increasing or generating the activity of a gene product with the activity of a gene product conferring the activity of “Chaperone protein CIpB”, especially from Azotobacter vinelandii or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said AvinDRAFT_{—}6093, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said AvinDRAFT_{—}6093, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said AvinDRAFT_{—}6093, or a functional equivalent or a homolog thereof as depicted in column 8 of Table II, application no. 5, preferably a homolog or functional equivalent as depicted in column 8 of Table II B, application no. 5, and being depicted in the same respective line as said AvinDRAFT_{—}6093, and preferably the activity is increased non-targeted.

Accordingly, in one embodiment, the molecule which activity is to be increased in the process of the invention is the gene product with an activity as a “Chaperone protein CIpB”, preferably it is the molecule of section (a) or (b) of this paragraph.

In particular, it was observed that in plants, especially in Arabidopsis thaliana, increasing or generating the activity of a gene product non-targeted with the activity of a “Chaperone protein CIpB”, preferably being encoded by a gene comprising the nucleic acid sequence SEQ ID NO. 33596, preferably the coding region thereof, conferred the production of or the increase in tryptophan compared with the wild type control.

The nucleic acid sequence of AvinDRAFT_{—}6679 from Azotobacter vinelandii, e.g. as shown in the respective line in column 5 of Table I, application no. 5, has been published in D. And the activity of the gene product thereof is the activity of electron transfer flavoprotein subunit beta.

Accordingly, in one embodiment, the process of the present invention for producing tryptophan in a non-human organism, like a microorganism or a plant or a part thereof, comprises increasing or generating the activity of a gene product with the activity of a gene product conferring the activity of “electron transfer flavoprotein subunit beta”, especially from Azotobacter vinelandii or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said AvinDRAFT_{—}6679, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said AvinDRAFT_{—}6679, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said AvinDRAFT_{—}6679, or a functional equivalent or a homolog thereof as depicted in column 8 of Table II, application no. 5, preferably a homolog or functional equivalent as depicted in column 8 of Table II B, application no. 5, and being depicted in the same respective line as said AvinDRAFT_{—}6679, and preferably the activity is increased non-targeted.

Accordingly, in one embodiment, the molecule which activity is to be increased in the process of the invention is the gene product with an activity as a “electron transfer flavoprotein subunit beta”, preferably it is the molecule of section (a) or (b) of this paragraph.

In particular, it was observed that in plants, especially in Arabidopsis thaliana, increasing or generating the activity of a gene product non-targeted with the activity of a “electron transfer flavoprotein subunit beta”, preferably being encoded by a gene comprising the nucleic acid sequence SEQ ID NO. 74729, preferably the coding region thereof, conferred the production of or the increase in tryptophan compared with the wild type control.

The nucleic acid sequence of B0004 from Escherichia coli, e.g. as shown in the respective line in column 5 of Table I, application no. 5, has been published in B. And the activity of the gene product thereof is the activity of threonine synthase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “threonine synthase”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B0004, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B0004, and preferably the activity is increased plastidic, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B0004, or a functional equivalent or a homolog thereof as depicted in column 8 of Table II, application no. 5, preferably a homolog or functional equivalent as depicted in column 8 of Table II B, application no. 5, and being depicted in the same respective line as said B0004, and preferably the activity is increased plastidic.

Accordingly, in one embodiment, the molecule which activity is to be increased in the process of the invention is the gene product with an activity as a “threonine synthase”, preferably it is the molecule of section (a) or (b) of this paragraph.

In particular, it was observed that in plants, especially in Arabidopsis thaliana, increasing or generating the activity of a gene product plastidic with the activity of a “threonine synthase”, preferably being encoded by a gene comprising the nucleic acid sequence SEQ ID NO. 34889, preferably the coding region thereof, conferred the production of or the increase in tryptophan compared with the wild type control.

The nucleic acid sequence of B0008 from Escherichia coli, e.g. as shown in the respective line in column 5 of Table I, application no. 5, has been published in B. And the activity of the gene product thereof is the activity of transaldolase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “transaldolase”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B0008, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B0008, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B0008, or a functional equivalent or a homolog thereof as depicted in column 8 of Table II, application no. 5, preferably a homolog or functional equivalent as depicted in column 8 of Table II B, application no. 5, and being depicted in the same respective line as said B0008, and preferably the activity is increased non-targeted.

Accordingly, in one embodiment, the molecule which activity is to be increased in the process of the invention is the gene product with an activity as a “transaldolase”, preferably it is the molecule of section (a) or (b) of this paragraph.

In particular, it was observed that in plants, especially in Arabidopsis thaliana, increasing or generating the activity of a gene product non-targeted with the activity of a “transaldolase”, preferably being encoded by a gene comprising the nucleic acid sequence SEQ ID NO. 98553, preferably the coding region thereof, conferred the production of or the increase in tryptophan compared with the wild type control.

The nucleic acid sequence of B0061 from Escherichia coli, e.g. as shown in the respective line in column 5 of Table I, application no. 5, has been published in B. And the activity of the gene product thereof is the activity of L-ribulose-5-phosphate 4-epimerase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “L-ribulose-5-phosphate 4-epimerase”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B0061, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B0061, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B0061, or a functional equivalent or a homolog thereof as depicted in column 8 of Table II, application no. 5, preferably a homolog or functional equivalent as depicted in column 8 of Table II B, application no. 5, and being depicted in the same respective line as said B0061, and preferably the activity is increased non-targeted.

Accordingly, in one embodiment, the molecule which activity is to be increased in the process of the invention is the gene product with an activity as a “L-ribulose-5-phosphate 4-epimerase”, preferably it is the molecule of section (a) or (b) of this paragraph.

In particular, it was observed that in plants, especially in Arabidopsis thaliana, increasing or generating the activity of a gene product non-targeted with the activity of a “L-ribulose-5-phosphate 4-epimerase”, preferably being encoded by a gene comprising the nucleic acid sequence SEQ ID NO. 35204, preferably the coding region thereof, conferred the production of or the increase in tryptophan compared with the wild type control.

The nucleic acid sequence of B0124 from Escherichia coli, e.g. as shown in the respective line in column 5 of Table I, application no. 5, has been published in B. And the activity of the gene product thereof is the activity of glucose dehydrogenase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “glucose dehydrogenase”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B0124, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B0124, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B0124, or a functional equivalent or a homolog thereof as depicted in column 8 of Table II, application no. 5, preferably a homolog or functional equivalent as depicted in column 8 of Table II B, application no. 5, and being depicted in the same respective line as said B0124, and preferably the activity is increased non-targeted.

Accordingly, in one embodiment, the molecule which activity is to be increased in the process of the invention is the gene product with an activity as a “glucose dehydrogenase”, preferably it is the molecule of section (a) or (b) of this paragraph.

In particular, it was observed that in plants, especially in Arabidopsis thaliana, increasing or generating the activity of a gene product non-targeted with the activity of a “glucose dehydrogenase”, preferably being encoded by a gene comprising the nucleic acid sequence SEQ ID NO. 35482, preferably the coding region thereof, conferred the production of or the increase in tryptophan compared with the wild type control.

The nucleic acid sequence of B0449 from Escherichia coli, e.g. as shown in the respective line in column 5 of Table I, application no. 5, has been published in B. And the activity of the gene product thereof is the activity of ATP-binding component of a transport system.

Accordingly, in one embodiment, the process of the present invention for producing tryptophan in a non-human organism, like a microorganism or a plant or a part thereof, comprises increasing or generating the activity of a gene product with the activity of a gene product conferring the activity of “ATP-binding component of a transport system”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B0449, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B0449, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B0449, or a functional equivalent or a homolog thereof as depicted in column 8 of Table II, application no. 5, preferably a homolog or functional equivalent as depicted in column 8 of Table II B, application no. 5, and being depicted in the same respective line as said B0449, and preferably the activity is increased non-targeted.

Accordingly, in one embodiment, the molecule which activity is to be increased in the process of the invention is the gene product with an activity as a “ATP-binding component of a transport system”, preferably it is the molecule of section (a) or (b) of this paragraph.

In particular, it was observed that in plants, especially in Arabidopsis thaliana, increasing or generating the activity of a gene product non-targeted with the activity of a “ATP-binding component of a transport system”, preferably being encoded by a gene comprising the nucleic acid sequence SEQ ID NO. 7333, preferably the coding region thereof, conferred the production of or the increase in tryptophan compared with the wild type control.

The nucleic acid sequence of B0593 from Escherichia coli, e.g. as shown in the respective line in column 5 of Table I, application no. 5, has been published in B. And the activity of the gene product thereof is the activity of isochorismate synthase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “isochorismate synthase”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B0593, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B0593, and preferably the activity is increased plastidic, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B0593, or a functional equivalent or a homolog thereof as depicted in column 8 of Table II, application no. 5, preferably a homolog or functional equivalent as depicted in column 8 of Table II B, application no. 5, and being depicted in the same respective line as said B0593, and preferably the activity is increased plastidic.

Accordingly, in one embodiment, the molecule which activity is to be increased in the process of the invention is the gene product with an activity as a “isochorismate synthase”, preferably it is the molecule of section (a) or (b) of this paragraph.

In particular, it was observed that in plants, especially in Arabidopsis thaliana, increasing or generating the activity of a gene product plastidic with the activity of a “isochorismate synthase”, preferably being encoded by a gene comprising the nucleic acid sequence SEQ ID NO. 35967, preferably the coding region thereof, conferred the production of or the increase in tryptophan compared with the wild type control.

The nucleic acid sequence of B0885 from Escherichia coli, e.g. as shown in the respective line in column 5 of Table I, application no. 5, has been published in B. And the activity of the gene product thereof is the activity of Leucyl/phenylalanyl-tRNA-protein transferase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “Leucyl/phenylalanyl-tRNA-protein transferase”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B0885, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B0885, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B0885, or a functional equivalent or a homolog thereof as depicted in column 8 of Table II, application no. 5, preferably a homolog or functional equivalent as depicted in column 8 of Table II B, application no. 5, and being depicted in the same respective line as said B0885, and preferably the activity is increased non-targeted.

Accordingly, in one embodiment, the molecule which activity is to be increased in the process of the invention is the gene product with an activity as a “Leucyl/phenylalanyl-tRNA-protein transferase”, preferably it is the molecule of section (a) or (b) of this paragraph.

In particular, it was observed that in plants, especially in Arabidopsis thaliana, increasing or generating the activity of a gene product non-targeted with the activity of a “Leucyl/phenylalanyl-tRNA-protein transferase”, preferably being encoded by a gene comprising the nucleic acid sequence SEQ ID NO. 36489, preferably the coding region thereof, conferred the production of or the increase in tryptophan compared with the wild type control.

The nucleic acid sequence of B1163 from Escherichia coli, e.g. as shown in the respective line in column 5 of Table I, application no. 5, has been published in B. And the activity of the gene product thereof is the activity of b1163-protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “b1163-protein”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B1163, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B1163, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B1163, or a functional equivalent or a homolog thereof as depicted in column 8 of Table II, application no. 5, preferably a homolog or functional equivalent as depicted in column 8 of Table II B, application no. 5, and being depicted in the same respective line as said B1163, and preferably the activity is increased non-targeted.

Accordingly, in one embodiment, the molecule which activity is to be increased in the process of the invention is the gene product with an activity as a “b1163-protein”, preferably it is the molecule of section (a) or (b) of this paragraph.

In particular, it was observed that in plants, especially in Arabidopsis thaliana, increasing or generating the activity of a gene product non-targeted with the activity of a “b1163-protein”, preferably being encoded by a gene comprising the nucleic acid sequence SEQ ID NO. 37394, preferably the coding region thereof, conferred the production of or the increase in tryptophan compared with the wild type control.

The nucleic acid sequence of B1249 from Escherichia coli, e.g. as shown in the respective line in column 5 of Table I, application no. 5, has been published in B. And the activity of the gene product thereof is the activity of cardiolipin synthase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “cardiolipin synthase”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B1249, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B1249, and preferably the activity is increased plastidic, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B1249, or a functional equivalent or a homolog thereof as depicted in column 8 of Table II, application no. 5, preferably a homolog or functional equivalent as depicted in column 8 of Table II B, application no. 5, and being depicted in the same respective line as said B1249, and preferably the activity is increased plastidic.

Accordingly, in one embodiment, the molecule which activity is to be increased in the process of the invention is the gene product with an activity as a “cardiolipin synthase”, preferably it is the molecule of section (a) or (b) of this paragraph.

In particular, it was observed that in plants, especially in Arabidopsis thaliana, increasing or generating the activity of a gene product plastidic with the activity of a “cardiolipin synthase”, preferably being encoded by a gene comprising the nucleic acid sequence SEQ ID NO. 98778, preferably the coding region thereof, conferred the production of or the increase in tryptophan compared with the wild type control.

The nucleic acid sequence of B1262 from Escherichia coli, e.g. as shown in the respective line in column 5 of Table I, application no. 5, has been published in B. And the activity of the gene product thereof is the activity of tryptophan biosynthesis protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “tryptophan biosynthesis protein”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B1262, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B1262, and preferably the activity is increased plastidic, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B1262, or a functional equivalent or a homolog thereof as depicted in column 8 of Table II, application no. 5, preferably a homolog or functional equivalent as depicted in column 8 of Table II B, application no. 5, and being depicted in the same respective line as said B1262, and preferably the activity is increased plastidic.

Accordingly, in one embodiment, the molecule which activity is to be increased in the process of the invention is the gene product with an activity as a “tryptophan biosynthesis protein”, preferably it is the molecule of section (a) or (b) of this paragraph.

In particular, it was observed that in plants, especially in Arabidopsis thaliana, increasing or generating the activity of a gene product plastidic with the activity of a “tryptophan biosynthesis protein”, preferably being encoded by a gene comprising the nucleic acid sequence SEQ ID NO. 76157, preferably the coding region thereof, conferred the production of or the increase in tryptophan compared with the wild type control.

The nucleic acid sequence of B1300 from Escherichia coli, e.g. as shown in the respective line in column 5 of Table I, application no. 5, has been published in B. And the activity of the gene product thereof is the activity of aldehyde dehydrogenase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “aldehyde dehydrogenase”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B1300, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B1300, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B1300, or a functional equivalent or a homolog thereof as depicted in column 8 of Table II, application no. 5, preferably a homolog or functional equivalent as depicted in column 8 of Table II B, application no. 5, and being depicted in the same respective line as said B1300, and preferably the activity is increased non-targeted.

Accordingly, in one embodiment, the molecule which activity is to be increased in the process of the invention is the gene product with an activity as a “aldehyde dehydrogenase”, preferably it is the molecule of section (a) or (b) of this paragraph.

In particular, it was observed that in plants, especially in Arabidopsis thaliana, increasing or generating the activity of a gene product non-targeted with the activity of a “aldehyde dehydrogenase”, preferably being encoded by a gene comprising the nucleic acid sequence SEQ ID NO. 37807, preferably the coding region thereof, conferred the production of or the increase in tryptophan compared with the wild type control.

The nucleic acid sequence of B1346 from Escherichia coli, e.g. as shown in the respective line in column 5 of Table I, application no. 5, has been published in B. And the activity of the gene product thereof is the activity of b1346-protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “b1346-protein”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B1346, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B1346, and preferably the activity is increased plastidic, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B1346, or a functional equivalent or a homolog thereof as depicted in column 8 of Table II, application no. 5, preferably a homolog or functional equivalent as depicted in column 8 of Table II B, application no. 5, and being depicted in the same respective line as said B1346, and preferably the activity is increased plastidic.

Accordingly, in one embodiment, the molecule which activity is to be increased in the process of the invention is the gene product with an activity as a “b1346-protein”, preferably it is the molecule of section (a) or (b) of this paragraph.

In particular, it was observed that in plants, especially in Arabidopsis thaliana, increasing or generating the activity of a gene product plastidic with the activity of a “b1346-protein”, preferably being encoded by a gene comprising the nucleic acid sequence SEQ ID NO. 99188, preferably the coding region thereof, conferred the production of or the increase in tryptophan compared with the wild type control.

The nucleic acid sequence of B1522 from Escherichia coli, e.g. as shown in the respective line in column 5 of Table I, application no. 5, has been published in B. And the activity of the gene product thereof is the activity of b1522-protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “b1522-protein”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B1522, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B1522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B1522, or a functional equivalent or a homolog thereof as depicted in column 8 of Table II, application no. 5, preferably a homolog or functional equivalent as depicted in column 8 of Table II B, application no. 5, and being depicted in the same respective line as said B1522, and preferably the activity is increased non-targeted.

Accordingly, in one embodiment, the molecule which activity is to be increased in the process of the invention is the gene product with an activity as a “b1522-protein”, preferably it is the molecule of section (a) or (b) of this paragraph.

In particular, it was observed that in plants, especially in Arabidopsis thaliana, increasing or generating the activity of a gene product non-targeted with the activity of a “b1522-protein”, preferably being encoded by a gene comprising the nucleic acid sequence SEQ ID NO. 7947, preferably the coding region thereof, conferred the production of or the increase in tryptophan compared with the wild type control.

The nucleic acid sequence of B1796 from Escherichia coli, e.g. as shown in the respective line in column 5 of Table I, application no. 5, has been published in B. And the activity of the gene product thereof is the activity of b1796-protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “b1796-protein”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B1796, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B1796, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B1796, or a functional equivalent or a homolog thereof as depicted in column 8 of Table II, application no. 5, preferably a homolog or functional equivalent as depicted in column 8 of Table II B, application no. 5, and being depicted in the same respective line as said B1796, and preferably the activity is increased non-targeted.

Accordingly, in one embodiment, the molecule which activity is to be increased in the process of the invention is the gene product with an activity as a “b1796-protein”, preferably it is the molecule of section (a) or (b) of this paragraph.

In particular, it was observed that in plants, especially in Arabidopsis thaliana, increasing or generating the activity of a gene product non-targeted with the activity of a “b1796-protein”, preferably being encoded by a gene comprising the nucleic acid sequence SEQ ID NO. 99196, preferably the coding region thereof, conferred the production of or the increase in tryptophan compared with the wild type control. The nucleic acid sequence of B1886 from Escherichia coli, e.g. as shown in the respective line in column 5 of Table I, application no. 5, has been published in B. And the activity of the gene product thereof is the activity of methyl-accepting chemotaxis protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “methyl-accepting chemotaxis protein”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B1886, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B1886, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B1886, or a functional equivalent or a homolog thereof as depicted in column 8 of Table II, application no. 5, preferably a homolog or functional equivalent as depicted in column 8 of Table II B, application no. 5, and being depicted in the same respective line as said B1886, and preferably the activity is increased non-targeted.

Accordingly, in one embodiment, the molecule which activity is to be increased in the process of the invention is the gene product with an activity as a “methyl-accepting chemotaxis protein”, preferably it is the molecule of section (a) or (b) of this paragraph.

In particular, it was observed that in plants, especially in Arabidopsis thaliana, increasing or generating the activity of a gene product non-targeted with the activity of a “methyl-accepting chemotaxis protein”, preferably being encoded by a gene comprising the nucleic acid sequence SEQ ID NO. 99210, preferably the coding region thereof, conferred the production of or the increase in tryptophan compared with the wild type control.

The nucleic acid sequence of B1897 from Escherichia coli, e.g. as shown in the respective line in column 5 of Table I, application no. 5, has been published in B. And the activity of the gene product thereof is the activity of trehalose-phosphatase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “trehalose-phosphatase”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B1897, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B1897, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B1897, or a functional equivalent or a homolog thereof as depicted in column 8 of Table II, application no. 5, preferably a homolog or functional equivalent as depicted in column 8 of Table II B, application no. 5, and being depicted in the same respective line as said B1897, and preferably the activity is increased non-targeted.

Accordingly, in one embodiment, the molecule which activity is to be increased in the process of the invention is the gene product with an activity as a “trehalose-phosphatase”, preferably it is the molecule of section (a) or (b) of this paragraph.

In particular, it was observed that in plants, especially in Arabidopsis thaliana, increasing or generating the activity of a gene product non-targeted with the activity of a “trehalosephosphatase”, preferably being encoded by a gene comprising the nucleic acid sequence SEQ ID NO. 79056, preferably the coding region thereof, conferred the production of or the increase in tryptophan compared with the wild type control.

The nucleic acid sequence of B2023 from Escherichia coli, e.g. as shown in the respective line in column 5 of Table I, application no. 5, has been published in B. And the activity of the gene product thereof is the activity of imidazole glycerol phosphate synthase subunit.

Accordingly, in one embodiment, the process of the present invention for producing tryptophan in a non-human organism, like a microorganism or a plant or a part thereof, comprises increasing or generating the activity of a gene product with the activity of a gene product conferring the activity of “imidazole glycerol phosphate synthase subunit”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B2023, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B2023, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B2023, or a functional equivalent or a homolog thereof as depicted in column 8 of Table II, application no. 5, preferably a homolog or functional equivalent as depicted in column 8 of Table II B, application no. 5, and being depicted in the same respective line as said B2023, and preferably the activity is increased non-targeted.

Accordingly, in one embodiment, the molecule which activity is to be increased in the process of the invention is the gene product with an activity as a “imidazole glycerol phosphate synthase subunit”, preferably it is the molecule of section (a) or (b) of this paragraph.

In particular, it was observed that in plants, especially in Arabidopsis thaliana, increasing or generating the activity of a gene product non-targeted with the activity of a “imidazole glycerol phosphate synthase subunit”, preferably being encoded by a gene comprising the nucleic acid sequence SEQ ID NO. 99371, preferably the coding region thereof, conferred the production of or the increase in tryptophan compared with the wild type control.

The nucleic acid sequence of B2063 from Escherichia coli, e.g. as shown in the respective line in column 5 of Table I, application no. 5, has been published in B. And the activity of the gene product thereof is the activity of transport protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “transport protein”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B2063, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B2063, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B2063, or a functional equivalent or a homolog thereof as depicted in column 8 of Table II, application no. 5, preferably a homolog or functional equivalent as depicted in column 8 of Table II B, application no. 5, and being depicted in the same respective line as said B2063, and preferably the activity is increased non-targeted.

Accordingly, in one embodiment, the molecule which activity is to be increased in the process of the invention is the gene product with an activity as a “transport protein”, preferably it is the molecule of section (a) or (b) of this paragraph.

In particular, it was observed that in plants, especially in Arabidopsis thaliana, increasing or generating the activity of a gene product non-targeted with the activity of a “transport protein”, preferably being encoded by a gene comprising the nucleic acid sequence SEQ ID NO. 38947, preferably the coding region thereof, conferred the production of or the increase in tryptophan compared with the wild type control.

The nucleic acid sequence of B2285 from Escherichia coli, e.g. as shown in the respective line in column 5 of Table I, application no. 5, has been published in B. And the activity of the gene product thereof is the activity of ATP synthase subunit E.

Accordingly, in one embodiment, the process of the present invention for producing tryptophan in a non-human organism, like a microorganism or a plant or a part thereof, comprises increasing or generating the activity of a gene product with the activity of a gene product conferring the activity of “ATP synthase subunit E”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B2285, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B2285, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B2285, or a functional equivalent or a homolog thereof as depicted in column 8 of Table II, application no. 5, preferably a homolog or functional equivalent as depicted in column 8 of Table II B, application no. 5, and being depicted in the same respective line as said B2285, and preferably the activity is increased non-targeted.

Accordingly, in one embodiment, the molecule which activity is to be increased in the process of the invention is the gene product with an activity as a “ATP synthase subunit E”, preferably it is the molecule of section (a) or (b) of this paragraph.

In particular, it was observed that in plants, especially in Arabidopsis thaliana, increasing or generating the activity of a gene product non-targeted with the activity of a “ATP synthase subunit E”, preferably being encoded by a gene comprising the nucleic acid sequence SEQ ID NO. 99620, preferably the coding region thereof, conferred the production of or the increase in tryptophan compared with the wild type control.

The nucleic acid sequence of B2546 from Escherichia coli, e.g. as shown in the respective line in column 5 of Table I, application no. 5, has been published in B. And the activity of the gene product thereof is the activity of putative transport system permease protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “putative transport system permease protein”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B2546, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B2546, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B2546, or a functional equivalent or a homolog thereof as depicted in column 8 of Table II, application no. 5, preferably a homolog or functional equivalent as depicted in column 8 of Table II B, application no. 5, and being depicted in the same respective line as said B2546, and preferably the activity is increased non-targeted.

Accordingly, in one embodiment, the molecule which activity is to be increased in the process of the invention is the gene product with an activity as a “putative transport system permease protein”, preferably it is the molecule of section (a) or (b) of this paragraph.

In particular, it was observed that in plants, especially in Arabidopsis thaliana, increasing or generating the activity of a gene product non-targeted with the activity of a “putative transport system permease protein”, preferably being encoded by a gene comprising the nucleic acid sequence SEQ ID NO. 99898, preferably the coding region thereof, conferred the production of or the increase in tryptophan compared with the wild type control.

The nucleic acid sequence of B2699 from Escherichia coli, e.g. as shown in the respective line in column 5 of Table I, application no. 5, has been published in B. And the activity of the gene product thereof is the activity of recombinase A.

Accordingly, in one embodiment, the process of the present invention for producing tryptophan in a non-human organism, like a microorganism or a plant or a part thereof, comprises increasing or generating the activity of a gene product with the activity of a gene product conferring the activity of “recombinase A”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B2699, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B2699, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B2699, or a functional equivalent or a homolog thereof as depicted in column 8 of Table II, application no. 5, preferably a homolog or functional equivalent as depicted in column 8 of Table II B, application no. 5, and being depicted in the same respective line as said B2699, and preferably the activity is increased non-targeted.

Accordingly, in one embodiment, the molecule which activity is to be increased in the process of the invention is the gene product with an activity as a “recombinase A”, preferably it is the molecule of section (a) or (b) of this paragraph.

In particular, it was observed that in plants, especially in Arabidopsis thaliana, increasing or generating the activity of a gene product non-targeted with the activity of a “recombinase A”, preferably being encoded by a gene comprising the nucleic acid sequence SEQ ID NO. 100105, preferably the coding region thereof, conferred the production of or the increase in tryptophan compared with the wild type control.

The nucleic acid sequence of B2739 from Escherichia coli, e.g. as shown in the respective line in column 5 of Table I, application no. 5, has been published in B. And the activity of the gene product thereof is the activity of b2739-protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “b2739-protein”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B2739, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B2739, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B2739, or a functional equivalent or a homolog thereof as depicted in column 8 of Table II, application no. 5, preferably a homolog or functional equivalent as depicted in column 8 of Table II B, application no. 5, and being depicted in the same respective line as said B2739, and preferably the activity is increased non-targeted.

Accordingly, in one embodiment, the molecule which activity is to be increased in the process of the invention is the gene product with an activity as a “b2739-protein”, preferably it is the molecule of section (a) or (b) of this paragraph.

In particular, it was observed that in plants, especially in Arabidopsis thaliana, increasing or generating the activity of a gene product non-targeted with the activity of a “b2739-protein”, preferably being encoded by a gene comprising the nucleic acid sequence SEQ ID NO. 80906, preferably the coding region thereof, conferred the production of or the increase in tryptophan compared with the wild type control.

The nucleic acid sequence of B2849 from Escherichia coli, e.g. as shown in the respective line in column 5 of Table I, application no. 5, has been published in B. And the activity of the gene product thereof is the activity of b2849-protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “b2849-protein”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B2849, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B2849, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B2849, or a functional equivalent or a homolog thereof as depicted in column 8 of Table II, application no. 5, preferably a homolog or functional equivalent as depicted in column 8 of Table II B, application no. 5, and being depicted in the same respective line as said B2849, and preferably the activity is increased non-targeted.

Accordingly, in one embodiment, the molecule which activity is to be increased in the process of the invention is the gene product with an activity as a “b2849-protein”, preferably it is the molecule of section (a) or (b) of this paragraph.

In particular, it was observed that in plants, especially in Arabidopsis thaliana, increasing or generating the activity of a gene product non-targeted with the activity of a “b2849-protein”, preferably being encoded by a gene comprising the nucleic acid sequence SEQ ID NO. 81980, preferably the coding region thereof, conferred the production of or the increase in tryptophan compared with the wild type control.

The nucleic acid sequence of B3142 from Escherichia coli, e.g. as shown in the respective line in column 5 of Table I, application no. 5, has been published in B. And the activity of the gene product thereof is the activity of fimbrial-like protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “fimbrial-like protein”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B3142, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B3142, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B3142, or a functional equivalent or a homolog thereof as depicted in column 8 of Table II, application no. 5, preferably a homolog or functional equivalent as depicted in column 8 of Table II B, application no. 5, and being depicted in the same respective line as said B3142, and preferably the activity is increased non-targeted.

Accordingly, in one embodiment, the molecule which activity is to be increased in the process of the invention is the gene product with an activity as a “fimbrial-like protein”, preferably it is the molecule of section (a) or (b) of this paragraph.

In particular, it was observed that in plants, especially in Arabidopsis thaliana, increasing or generating the activity of a gene product non-targeted with the activity of a “fimbrial-like protein”, preferably being encoded by a gene comprising the nucleic acid sequence SEQ ID NO. 100543, preferably the coding region thereof, conferred the production of or the increase in tryptophan compared with the wild type control.

The nucleic acid sequence of B3262 from Escherichia coli, e.g. as shown in the respective line in column 5 of Table I, application no. 5, has been published in B. And the activity of the gene product thereof is the activity of methyltransferase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “methyltransferase”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B3262, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B3262, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B3262, or a functional equivalent or a homolog thereof as depicted in column 8 of Table II, application no. 5, preferably a homolog or functional equivalent as depicted in column 8 of Table II B, application no. 5, and being depicted in the same respective line as said B3262, and preferably the activity is increased non-targeted.

Accordingly, in one embodiment, the molecule which activity is to be increased in the process of the invention is the gene product with an activity as a “methyltransferase”, preferably it is the molecule of section (a) or (b) of this paragraph.

In particular, it was observed that in plants, especially in Arabidopsis thaliana, increasing or generating the activity of a gene product non-targeted with the activity of a “methyltransferase”, preferably being encoded by a gene comprising the nucleic acid sequence SEQ ID NO. 42502, preferably the coding region thereof, conferred the production of or the increase in tryptophan compared with the wild type control.

The nucleic acid sequence of B3845 from Escherichia coli, e.g. as shown in the respective line in column 5 of Table I, application no. 5, has been published in B. And the activity of the gene product thereof is the activity of 3-ketoacyl-CoA thiolase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “3-ketoacyl-CoA thiolase”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B3845, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B3845, and preferably the activity is increased plastidic, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B3845, or a functional equivalent or a homolog thereof as depicted in column 8 of Table II, application no. 5, preferably a homolog or functional equivalent as depicted in column 8 of Table II B, application no. 5, and being depicted in the same respective line as said B3845, and preferably the activity is increased plastidic.

Accordingly, in one embodiment, the molecule which activity is to be increased in the process of the invention is the gene product with an activity as a “3-ketoacyl-CoA thiolase”, preferably it is the molecule of section (a) or (b) of this paragraph.

In particular, it was observed that in plants, especially in Arabidopsis thaliana, increasing or generating the activity of a gene product plastidic with the activity of a “3-ketoacyl-CoA thiolase”, preferably being encoded by a gene comprising the nucleic acid sequence SEQ ID NO. 100675, preferably the coding region thereof, conferred the production of or the increase in tryptophan compared with the wild type control.

The nucleic acid sequence of B3870 from Escherichia coli, e.g. as shown in the respective line in column 5 of Table I, application no. 5, has been published in B. And the activity of the gene product thereof is the activity of glutamine synthetase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “glutamine synthetase”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B3870, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B3870, and preferably the activity is increased plastidic, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B3870, or a functional equivalent or a homolog thereof as depicted in column 8 of Table II, application no. 5, preferably a homolog or functional equivalent as depicted in column 8 of Table II B, application no. 5, and being depicted in the same respective line as said B3870, and preferably the activity is increased plastidic.

Accordingly, in one embodiment, the molecule which activity is to be increased in the process of the invention is the gene product with an activity as a “glutamine synthetase”, preferably it is the molecule of section (a) or (b) of this paragraph.

In particular, it was observed that in plants, especially in Arabidopsis thaliana, increasing or generating the activity of a gene product plastidic with the activity of a “glutamine synthetase”, preferably being encoded by a gene comprising the nucleic acid sequence SEQ ID NO. 101327, preferably the coding region thereof, conferred the production of or the increase in tryptophan compared with the wild type control.

The nucleic acid sequence of B3989 from Escherichia coli, e.g. as shown in the respective line in column 5 of Table I, application no. 5, has been published in B. And the activity of the gene product thereof is the activity of b3989-protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “b3989-protein”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B3989, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B3989, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B3989, or a functional equivalent or a homolog thereof as depicted in column 8 of Table II, application no. 5, preferably a homolog or functional equivalent as depicted in column 8 of Table II B, application no. 5, and being depicted in the same respective line as said B3989, and preferably the activity is increased non-targeted.

Accordingly, in one embodiment, the molecule which activity is to be increased in the process of the invention is the gene product with an activity as a “b3989-protein”, preferably it is the molecule of section (a) or (b) of this paragraph.

In particular, it was observed that in plants, especially in Arabidopsis thaliana, increasing or generating the activity of a gene product non-targeted with the activity of a “b3989-protein”, preferably being encoded by a gene comprising the nucleic acid sequence SEQ ID NO. 44372, preferably the coding region thereof, conferred the production of or the increase in tryptophan compared with the wild type control.

The nucleic acid sequence of B4029 from Escherichia coli, e.g. as shown in the respective line in column 5 of Table I, application no. 5, has been published in B. And the activity of the gene product thereof is the activity of b4029-protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “b4029-protein”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B4029, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B4029, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B4029, or a functional equivalent or a homolog thereof as depicted in column 8 of Table II, application no. 5, preferably a homolog or functional equivalent as depicted in column 8 of Table II B, application no. 5, and being depicted in the same respective line as said B4029, and preferably the activity is increased non-targeted.

Accordingly, in one embodiment, the molecule which activity is to be increased in the process of the invention is the gene product with an activity as a “b4029-protein”, preferably it is the molecule of section (a) or (b) of this paragraph.

In particular, it was observed that in plants, especially in Arabidopsis thaliana, increasing or generating the activity of a gene product non-targeted with the activity of a “b4029-protein”, preferably being encoded by a gene comprising the nucleic acid sequence SEQ ID NO. 10740, preferably the coding region thereof, conferred the production of or the increase in tryptophan compared with the wild type control.

The nucleic acid sequence of B4121 from Escherichia coli, e.g. as shown in the respective line in column 5 of Table I, application no. 5, has been published in B. And the activity of the gene product thereof is the activity of b4121-protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “b4121-protein”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B4121, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B4121, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B4121, or a functional equivalent or a homolog thereof as depicted in column 8 of Table II, application no. 5, preferably a homolog or functional equivalent as depicted in column 8 of Table II B, application no. 5, and being depicted in the same respective line as said B4121, and preferably the activity is increased non-targeted.

Accordingly, in one embodiment, the molecule which activity is to be increased in the process of the invention is the gene product with an activity as a “b4121-protein”, preferably it is the molecule of section (a) or (b) of this paragraph.

In particular, it was observed that in plants, especially in Arabidopsis thaliana, increasing or generating the activity of a gene product non-targeted with the activity of a “b4121-protein”, preferably being encoded by a gene comprising the nucleic acid sequence SEQ ID NO. 44609, preferably the coding region thereof, conferred the production of or the increase in tryptophan compared with the wild type control.

The nucleic acid sequence of B4256 from Escherichia coli, e.g. as shown in the respective line in column 5 of Table I, application no. 5, has been published in B. And the activity of the gene product thereof is the activity of acetyltransferase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said B4256, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said B4256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said B4256, or a functional equivalent or a homolog thereof as depicted in column 8 of Table II, application no. 5, preferably a homolog or functional equivalent as depicted in column 8 of Table II B, application no. 5, and being depicted in the same respective line as said B4256, and preferably the activity is increased non-targeted.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 45321, preferably the coding region thereof, conferred the production of or the increase in tryptophan compared with the wild type control.

The nucleic acid sequence of GM02LC17556 from Glycine max, e.g. as shown in the respective line in column 5 of Table I, application no. 5, is unpublished. And the activity of the gene product thereof is the activity of RNA binding protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “RNA binding protein”, especially from Glycine max or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said GM02LC17556, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said GM02LC17556, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said GM02LC17556, or a functional equivalent or a homolog thereof as depicted in column 8 of Table II, application no. 5, preferably a homolog or functional equivalent as depicted in column 8 of Table II B, application no. 5, and being depicted in the same respective line as said GM02LC17556, and preferably the activity is increased non-targeted.

Accordingly, in one embodiment, the molecule which activity is to be increased in the process of the invention is the gene product with an activity as a “RNA binding protein”, preferably it is the molecule of section (a) or (b) of this paragraph.

In particular, it was observed that in plants, especially in Arabidopsis thaliana, increasing or generating the activity of a gene product non-targeted with the activity of a “RNA binding protein”, preferably being encoded by a gene comprising the nucleic acid sequence SEQ ID NO. 46515, preferably the coding region thereof, conferred the production of or the increase in tryptophan compared with the wild type control.

The nucleic acid sequence of GM02LC21368 from Glycine max, e.g. as shown in the respective line in column 5 of Table I, application no. 5, is unpublished. And the activity of the gene product thereof is the activity of transcription factor.

Accordingly, in one embodiment, the process of the present invention for producing tryptophan in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said GM02LC21368, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said GM02LC21368, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said GM02LC21368, or a functional equivalent or a homolog thereof as depicted in column 8 of Table II, application no. 5, preferably a homolog or functional equivalent as depicted in column 8 of Table II B, application no. 5, and being depicted in the same respective line as said GM02LC21368, and preferably the activity is increased non-targeted.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 84198, preferably the coding region thereof, conferred the production of or the increase in tryptophan compared with the wild type control.

The nucleic acid sequence of S_pp020029328r from Physcomitrella patens, e.g. as shown in the respective line in column 5 of Table I, application no. 5, is unpublished. And the activity of the gene product thereof is the activity of choline kinase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “choline kinase”, especially from Physcomitrella patens or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said S_pp020029328r, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said S_pp020029328r, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said S_pp020029328r, or a functional equivalent or a homolog thereof as depicted in column 8 of Table II, application no. 5, preferably a homolog or functional equivalent as depicted in column 8 of Table II B, application no. 5, and being depicted in the same respective line as said S_pp020029328r, and preferably the activity is increased non-targeted.

Accordingly, in one embodiment, the molecule which activity is to be increased in the process of the invention is the gene product with an activity as a “choline kinase”, preferably it is the molecule of section (a) or (b) of this paragraph.

In particular, it was observed that in plants, especially in Arabidopsis thaliana, increasing or generating the activity of a gene product non-targeted with the activity of a “choline kinase”, preferably being encoded by a gene comprising the nucleic acid sequence SEQ ID NO. 101780, preferably the coding region thereof, conferred the production of or the increase in tryptophan compared with the wild type control.

The nucleic acid sequence of Sll0248 from Synechocystis sp., e.g. as shown in the respective line in column 5 of Table I, application no. 5, has been published in C. And the activity of the gene product thereof is the activity of flavodoxin.

Accordingly, in one embodiment, the process of the present invention for producing tryptophan in a non-human organism, like a microorganism or a plant or a part thereof, comprises increasing or generating the activity of a gene product with the activity of a gene product conferring the activity of “flavodoxin”, especially from Synechocystis sp. or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said Sll0248, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said Sll0248, and preferably the activity is increased plastidic, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said Sll0248, or a functional equivalent or a homolog thereof as depicted in column 8 of Table II, application no. 5, preferably a homolog or functional equivalent as depicted in column 8 of Table II B, application no. 5, and being depicted in the same respective line as said Sll0248, and preferably the activity is increased plastidic.

Accordingly, in one embodiment, the molecule which activity is to be increased in the process of the invention is the gene product with an activity as a “flavodoxin”, preferably it is the molecule of section (a) or (b) of this paragraph.

In particular, it was observed that in plants, especially in Arabidopsis thaliana, increasing or generating the activity of a gene product plastidic with the activity of a “flavodoxin”, preferably being encoded by a gene comprising the nucleic acid sequence SEQ ID NO. 49143, preferably the coding region thereof, conferred the production of or the increase in tryptophan compared with the wild type control.

The nucleic acid sequence of Sll0521 from Synechocystis sp., e.g. as shown in the respective line in column 5 of Table I, application no. 5, has been published in C. And the activity of the gene product thereof is the activity of NAD(P)H-quinone oxidoreductase subunit.

Accordingly, in one embodiment, the process of the present invention for producing tryptophan in a non-human organism, like a microorganism or a plant or a part thereof, comprises increasing or generating the activity of a gene product with the activity of a gene product conferring the activity of “NAD(P)H-quinone oxidoreductase subunit”, especially from Synechocystis sp. or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said Sll0521, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said Sll0521, and preferably the activity is increased plastidic, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said Sll0521, or a functional equivalent or a homolog thereof as depicted in column 8 of Table II, application no. 5, preferably a homolog or functional equivalent as depicted in column 8 of Table II B, application no. 5, and being depicted in the same respective line as said Sll0521, and preferably the activity is increased plastidic.

Accordingly, in one embodiment, the molecule which activity is to be increased in the process of the invention is the gene product with an activity as a “NAD(P)H-quinone oxidoreductase subunit”, preferably it is the molecule of section (a) or (b) of this paragraph.

In particular, it was observed that in plants, especially in Arabidopsis thaliana, increasing or generating the activity of a gene product plastidic with the activity of a “NAD(P)H-quinone oxidoreductase subunit”, preferably being encoded by a gene comprising the nucleic acid sequence SEQ ID NO. 50339, preferably the coding region thereof, conferred the production of or the increase in tryptophan compared with the wild type control.

The nucleic acid sequence of Sll0833 from Synechocystis sp., e.g. as shown in the respective line in column 5 of Table I, application no. 5, has been published in C. And the activity of the gene product thereof is the activity of binding-protein-dependent transport systems inner membrane component.

Accordingly, in one embodiment, the process of the present invention for producing tryptophan in a non-human organism, like a microorganism or a plant or a part thereof, comprises increasing or generating the activity of a gene product with the activity of a gene product conferring the activity of “binding-protein-dependent transport systems inner membrane component”, especially from Synechocystis sp. or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said Sll0833, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said Sll0833, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said Sll0833, or a functional equivalent or a homolog thereof as depicted in column 8 of Table II, application no. 5, preferably a homolog or functional equivalent as depicted in column 8 of Table II B, application no. 5, and being depicted in the same respective line as said Sll0833, and preferably the activity is increased non-targeted.

Accordingly, in one embodiment, the molecule which activity is to be increased in the process of the invention is the gene product with an activity as a “binding-protein-dependent transport systems inner membrane component”, preferably it is the molecule of section (a) or (b) of this paragraph.

In particular, it was observed that in plants, especially in Arabidopsis thaliana, increasing or generating the activity of a gene product non-targeted with the activity of a “binding-proteindependent transport systems inner membrane component”, preferably being encoded by a gene comprising the nucleic acid sequence SEQ ID NO. 101707, preferably the coding region thereof, conferred the production of or the increase in tryptophan compared with the wild type control.

The nucleic acid sequence of Sll1031 from Synechocystis sp., e.g. as shown in the respective line in column 5 of Table I, application no. 5, has been published in C. And the activity of the gene product thereof is the activity of carbon dioxide concentrating mechanism protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “carbon dioxide concentrating mechanism protein”, especially from Synechocystis sp. or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said Sll1031, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said Sll1031, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said Sll1031, or a functional equivalent or a homolog thereof as depicted in column 8 of Table II, application no. 5, preferably a homolog or functional equivalent as depicted in column 8 of Table II B, application no. 5, and being depicted in the same respective line as said Sll1031, and preferably the activity is increased mitochondrial.

Accordingly, in one embodiment, the molecule which activity is to be increased in the process of the invention is the gene product with an activity as a “carbon dioxide concentrating mechanism protein”, preferably it is the molecule of section (a) or (b) of this paragraph.

In particular, it was observed that in plants, especially in Arabidopsis thaliana, increasing or generating the activity of a gene product mitochondrial with the activity of a “carbon dioxide concentrating mechanism protein”, preferably being encoded by a gene comprising the nucleic acid sequence SEQ ID NO. 52634, preferably the coding region thereof, conferred the production of or the increase in tryptophan compared with the wild type control.

The nucleic acid sequence of Sll1450 from Synechocystis sp., e.g. as shown in the respective line in column 5 of Table I, application no. 5, has been published in C. And the activity of the gene product thereof is the activity of nitrate/nitrite transport protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “nitrate/nitrite transport protein”, especially from Synechocystis sp. or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said Sll1450, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said Sll1450, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said Sll1450, or a functional equivalent or a homolog thereof as depicted in column 8 of Table II, application no. 5, preferably a homolog or functional equivalent as depicted in column 8 of Table II B, application no. 5, and being depicted in the same respective line as said Sll1450, and preferably the activity is increased mitochondrial.

Accordingly, in one embodiment, the molecule which activity is to be increased in the process of the invention is the gene product with an activity as a “nitrate/nitrite transport protein”, preferably it is the molecule of section (a) or (b) of this paragraph.

In particular, it was observed that in plants, especially in Arabidopsis thaliana, increasing or generating the activity of a gene product mitochondrial with the activity of a “nitrate/nitrite transport protein”, preferably being encoded by a gene comprising the nucleic acid sequence SEQ ID NO. 54337, preferably the coding region thereof, conferred the production of or the increase in tryptophan compared with the wild type control.

The nucleic acid sequence of Sll1917 from Synechocystis sp., e.g. as shown in the respective line in column 5 of Table I, application no. 5, has been published in C. And the activity of the gene product thereof is the activity of coproporphyrinogen III oxidase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “coproporphyrinogen III oxidase”, especially from Synechocystis sp. or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said Sll1917, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said Sll1917, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said Sll1917, or a functional equivalent or a homolog thereof as depicted in column 8 of Table II, application no. 5, preferably a homolog or functional equivalent as depicted in column 8 of Table II B, application no. 5, and being depicted in the same respective line as said Sll1917, and preferably the activity is increased mitochondrial.

Accordingly, in one embodiment, the molecule which activity is to be increased in the process of the invention is the gene product with an activity as a “coproporphyrinogen III oxidase”, preferably it is the molecule of section (a) or (b) of this paragraph.

In particular, it was observed that in plants, especially in Arabidopsis thaliana, increasing or generating the activity of a gene product mitochondrial with the activity of a “coproporphyrinogen III oxidase”, preferably being encoded by a gene comprising the nucleic acid sequence SEQ ID NO. 11471, preferably the coding region thereof, conferred the production of or the increase in tryptophan compared with the wild type control. The nucleic acid sequence of Slr0239 from Synechocystis sp., e.g. as shown in the respective line in column 5 of Table I, application no. 5, has been published in C. And the activity of the gene product thereof is the activity of precorrin methylase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “precorrin methylase”, especially from Synechocystis sp. or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said Slr0239, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said Slr0239, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said Slr0239, or a functional equivalent or a homolog thereof as depicted in column 8 of Table II, application no. 5, preferably a homolog or functional equivalent as depicted in column 8 of Table II B, application no. 5, and being depicted in the same respective line as said Slr0239, and preferably the activity is increased mitochondrial.

Accordingly, in one embodiment, the molecule which activity is to be increased in the process of the invention is the gene product with an activity as a “precorrin methylase”, preferably it is the molecule of section (a) or (b) of this paragraph.

In particular, it was observed that in plants, especially in Arabidopsis thaliana, increasing or generating the activity of a gene product mitochondrial with the activity of a “precorrin methylase”, preferably being encoded by a gene comprising the nucleic acid sequence SEQ ID NO. 86540, preferably the coding region thereof, conferred the production of or the increase in tryptophan compared with the wild type control.

The nucleic acid sequence of Slr0721 from Synechocystis sp., e.g. as shown in the respective line in column 5 of Table I, application no. 5, has been published in C. And the activity of the gene product thereof is the activity of malate dehydrogenase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “malate dehydrogenase”, especially from Synechocystis sp. or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said Slr0721, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said Slr0721, and preferably the activity is increased plastidic, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said Slr0721, or a functional equivalent or a homolog thereof as depicted in column 8 of Table II, application no. 5, preferably a homolog or functional equivalent as depicted in column 8 of Table II B, application no. 5, and being depicted in the same respective line as said Slr0721, and preferably the activity is increased plastidic.

Accordingly, in one embodiment, the molecule which activity is to be increased in the process of the invention is the gene product with an activity as a “malate dehydrogenase”, preferably it is the molecule of section (a) or (b) of this paragraph.

In particular, it was observed that in plants, especially in Arabidopsis thaliana, increasing or generating the activity of a gene product plastidic with the activity of a “malate dehydrogenase”, preferably being encoded by a gene comprising the nucleic acid sequence SEQ ID NO. 87397, preferably the coding region thereof, conferred the production of or the increase in tryptophan compared with the wild type control.

The nucleic acid sequence of Slr0739 from Synechocystis sp., e.g. as shown in the respective line in column 5 of Table I, application no. 5, has been published in C. And the activity of the gene product thereof is the activity of geranylgeranyl pyrophosphate synthase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “geranylgeranyl pyrophosphate synthase”, especially from Synechocystis sp. or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said Slr0739, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said Slr0739, and preferably the activity is increased plastidic, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said Slr0739, or a functional equivalent or a homolog thereof as depicted in column 8 of Table II, application no. 5, preferably a homolog or functional equivalent as depicted in column 8 of Table II B, application no. 5, and being depicted in the same respective line as said Slr0739, and preferably the activity is increased plastidic.

Accordingly, in one embodiment, the molecule which activity is to be increased in the process of the invention is the gene product with an activity as a “geranylgeranyl pyrophosphate synthase”, preferably it is the molecule of section (a) or (b) of this paragraph.

In particular, it was observed that in plants, especially in Arabidopsis thaliana, increasing or generating the activity of a gene product plastidic with the activity of a “geranylgeranyl pyrophosphate synthase”, preferably being encoded by a gene comprising the nucleic acid sequence SEQ ID NO. 57235, preferably the coding region thereof, conferred the production of or the increase in tryptophan compared with the wild type control.

The nucleic acid sequence of Slr1107 from Synechocystis sp., e.g. as shown in the respective line in column 5 of Table I, application no. 5, has been published in C. And the activity of the gene product thereof is the activity of slr1107-protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “slr1107-protein”, especially from Synechocystis sp. or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said Slr1107, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said Slr1107, and preferably the activity is increased plastidic, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said Slr1107, or a functional equivalent or a homolog thereof as depicted in column 8 of Table II, application no. 5, preferably a homolog or functional equivalent as depicted in column 8 of Table II B, application no. 5, and being depicted in the same respective line as said Slr1107, and preferably the activity is increased plastidic.

Accordingly, in one embodiment, the molecule which activity is to be increased in the process of the invention is the gene product with an activity as a “slr1107-protein”, preferably it is the molecule of section (a) or (b) of this paragraph.

In particular, it was observed that in plants, especially in Arabidopsis thaliana, increasing or generating the activity of a gene product plastidic with the activity of a “slr1107-protein”, preferably being encoded by a gene comprising the nucleic acid sequence SEQ ID NO. 101776, preferably the coding region thereof, conferred the production of or the increase in tryptophan compared with the wild type control.

The nucleic acid sequence of Slr1739 from Synechocystis sp., e.g. as shown in the respective line in column 5 of Table I, application no. 5, has been published in C. And the activity of the gene product thereof is the activity of photosystem II protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “photosystem II protein”, especially from Synechocystis sp. or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said Slr1739, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said Slr1739, and preferably the activity is increased plastidic, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said Slr1739, or a functional equivalent or a homolog thereof as depicted in column 8 of Table II, application no. 5, preferably a homolog or functional equivalent as depicted in column 8 of Table II B, application no. 5, and being depicted in the same respective line as said Slr1739, and preferably the activity is increased plastidic.

Accordingly, in one embodiment, the molecule which activity is to be increased in the process of the invention is the gene product with an activity as a “photosystem II protein”, preferably it is the molecule of section (a) or (b) of this paragraph.

In particular, it was observed that in plants, especially in Arabidopsis thaliana, increasing or generating the activity of a gene product plastidic with the activity of a “photosystem II protein”, preferably being encoded by a gene comprising the nucleic acid sequence SEQ ID NO. 58731, preferably the coding region thereof, conferred the production of or the increase in tryptophan compared with the wild type control.

The nucleic acid sequence of Slr2124 from Synechocystis sp., e.g. as shown in the respective line in column 5 of Table I, application no. 5, has been published in C. And the activity of the gene product thereof is the activity of short-chain alcohol dehydrogenase family.

Accordingly, in one embodiment, the process of the present invention for producing tryptophan in a non-human organism, like a microorganism or a plant or a part thereof, comprises increasing or generating the activity of a gene product with the activity of a gene product conferring the activity of “short-chain alcohol dehydrogenase family”, especially from Synechocystis sp. or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said Slr2124, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said Slr2124, and preferably the activity is increased plastidic, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said Slr2124, or a functional equivalent or a homolog thereof as depicted in column 8 of Table II, application no. 5, preferably a homolog or functional equivalent as depicted in column 8 of Table II B, application no. 5, and being depicted in the same respective line as said Slr2124, and preferably the activity is increased plastidic.

Accordingly, in one embodiment, the molecule which activity is to be increased in the process of the invention is the gene product with an activity as a “short-chain alcohol dehydrogenase family”, preferably it is the molecule of section (a) or (b) of this paragraph.

In particular, it was observed that in plants, especially in Arabidopsis thaliana, increasing or generating the activity of a gene product plastidic with the activity of a “short-chain alcohol dehydrogenase family”, preferably being encoded by a gene comprising the nucleic acid sequence SEQ ID NO. 59370, preferably the coding region thereof, conferred the production of or the increase in tryptophan compared with the wild type control.

The nucleic acid sequence of TTC0019 from Thermus thermophilus, e.g. as shown in the respective line in column 5 of Table I, application no. 5, has been published in E. And the activity of the gene product thereof is the activity of Sec-independent protein translocase subunit.

Accordingly, in one embodiment, the process of the present invention for producing tryptophan in a non-human organism, like a microorganism or a plant or a part thereof, comprises increasing or generating the activity of a gene product with the activity of a gene product conferring the activity of “Sec-independent protein translocase subunit”, especially from Thermus thermophilus or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said TTC0019, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said TTC0019, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said TTC0019, or a functional equivalent or a homolog thereof as depicted in column 8 of Table II, application no. 5, preferably a homolog or functional equivalent as depicted in column 8 of Table II B, application no. 5, and being depicted in the same respective line as said TTC0019, and preferably the activity is increased non-targeted.

Accordingly, in one embodiment, the molecule which activity is to be increased in the process of the invention is the gene product with an activity as a “Sec-independent protein translocase subunit”, preferably it is the molecule of section (a) or (b) of this paragraph.

In particular, it was observed that in plants, especially in Arabidopsis thaliana, increasing or generating the activity of a gene product non-targeted with the activity of a “Sec-independent protein translocase subunit”, preferably being encoded by a gene comprising the nucleic acid sequence SEQ ID NO. 12698, preferably the coding region thereof, conferred the production of or the increase in tryptophan compared with the wild type control.

The nucleic acid sequence of Ybr249c from Saccharomyces cerevisiae, e.g. as shown in the respective line in column 5 of Table I, application no. 5, has been published in A. And the activity of the gene product thereof is the activity of 3-deoxy-7-phosphoheptulonate synthase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “3-deoxy-7-phosphoheptulonate synthase”, especially from Saccharomyces cerevisiae or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said Ybr249c, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said Ybr249c, and preferably the activity is increased plastidic, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said Ybr249c, or a functional equivalent or a homolog thereof as depicted in column 8 of Table II, application no. 5, preferably a homolog or functional equivalent as depicted in column 8 of Table II B, application no. 5, and being depicted in the same respective line as said Ybr249c, and preferably the activity is increased plastidic.

Accordingly, in one embodiment, the molecule which activity is to be increased in the process of the invention is the gene product with an activity as a “3-deoxy-7-phosphoheptulonate synthase”, preferably it is the molecule of section (a) or (b) of this paragraph.

In particular, it was observed that in plants, especially in Arabidopsis thaliana, increasing or generating the activity of a gene product plastidic with the activity of a “3-deoxy-7-phosphoheptulonate synthase”, preferably being encoded by a gene comprising the nucleic acid sequence SEQ ID NO. 88879, preferably the coding region thereof, conferred the production of or the increase in tryptophan compared with the wild type control.

The nucleic acid sequence of Ydr046c from Saccharomyces cerevisiae, e.g. as shown in the respective line in column 5 of Table I, application no. 5, has been published in A. And the activity of the gene product thereof is the activity of branched-chain amino acid permease.

Accordingly, in one embodiment, the process of the present invention for producing tryptophan in a non-human organism, like a microorganism or a plant or a part thereof, comprises increasing or generating the activity of a gene product with the activity of a gene product conferring the activity of “branched-chain amino acid permease”, especially from Saccharomyces cerevisiae or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said Ydr046c, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said Ydr046c, and preferably the activity is increased plastidic, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said Ydr046c, or a functional equivalent or a homolog thereof as depicted in column 8 of Table II, application no. 5, preferably a homolog or functional equivalent as depicted in column 8 of Table II B, application no. 5, and being depicted in the same respective line as said Ydr046c, and preferably the activity is increased plastidic.

Accordingly, in one embodiment, the molecule which activity is to be increased in the process of the invention is the gene product with an activity as a “branched-chain amino acid permease”, preferably it is the molecule of section (a) or (b) of this paragraph.

In particular, it was observed that in plants, especially in Arabidopsis thaliana, increasing or generating the activity of a gene product plastidic with the activity of a “branched-chain amino acid permease”, preferably being encoded by a gene comprising the nucleic acid sequence SEQ ID NO. 63544, preferably the coding region thereof, conferred the production of or the increase in tryptophan compared with the wild type control.

The nucleic acid sequence of Yfl054c from Saccharomyces cerevisiae, e.g. as shown in the respective line in column 5 of Table I, application no. 5, has been published in A. And the activity of the gene product thereof is the activity of yfl054c-protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “yfl054c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said Yfl054c, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said Yfl054c, and preferably the activity is increased plastidic, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said Yfl054c, or a functional equivalent or a homolog thereof as depicted in column 8 of Table II, application no. 5, preferably a homolog or functional equivalent as depicted in column 8 of Table II B, application no. 5, and being depicted in the same respective line as said Yfl054c, and preferably the activity is increased plastidic.

Accordingly, in one embodiment, the molecule which activity is to be increased in the process of the invention is the gene product with an activity as a “yfl054c-protein”, preferably it is the molecule of section (a) or (b) of this paragraph.

In particular, it was observed that in plants, especially in Arabidopsis thaliana, increasing or generating the activity of a gene product plastidic with the activity of a “yfl054c-protein”, preferably being encoded by a gene comprising the nucleic acid sequence SEQ ID NO. 64148, preferably the coding region thereof, conferred the production of or the increase in tryptophan compared with the wild type control.

The nucleic acid sequence of Yhl032c from Saccharomyces cerevisiae, e.g. as shown in the respective line in column 5 of Table I, application no. 5, has been published in A. And the activity of the gene product thereof is the activity of glycerol kinase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “glycerol kinase”, especially from Saccharomyces cerevisiae or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said Yhl032c, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said Yhl032c, and preferably the activity is increased plastidic, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said Yhl032c, or a functional equivalent or a homolog thereof as depicted in column 8 of Table II, application no. 5, preferably a homolog or functional equivalent as depicted in column 8 of Table II B, application no. 5, and being depicted in the same respective line as said Yhl032c, and preferably the activity is increased plastidic.

Accordingly, in one embodiment, the molecule which activity is to be increased in the process of the invention is the gene product with an activity as a “glycerol kinase”, preferably it is the molecule of section (a) or (b) of this paragraph.

In particular, it was observed that in plants, especially in Arabidopsis thaliana, increasing or generating the activity of a gene product plastidic with the activity of a “glycerol kinase”, preferably being encoded by a gene comprising the nucleic acid sequence SEQ ID NO. 101855, preferably the coding region thereof, conferred the production of or the increase in tryptophan compared with the wild type control.

The nucleic acid sequence of Yhr137w from Saccharomyces cerevisiae, e.g. as shown in the respective line in column 5 of Table I, application no. 5, has been published in A. And the activity of the gene product thereof is the activity of aromatic amino acid aminotransferase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “aromatic amino acid aminotransferase”, especially from Saccharomyces cerevisiae or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said Yhr137w, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said Yhr137w, and preferably the activity is increased plastidic, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said Yhr137w, or a functional equivalent or a homolog thereof as depicted in column 8 of Table II, application no. 5, preferably a homolog or functional equivalent as depicted in column 8 of Table II B, application no. 5, and being depicted in the same respective line as said Yhr137w, and preferably the activity is increased plastidic.

Accordingly, in one embodiment, the molecule which activity is to be increased in the process of the invention is the gene product with an activity as a “aromatic amino acid aminotransferase”, preferably it is the molecule of section (a) or (b) of this paragraph.

In particular, it was observed that in plants, especially in Arabidopsis thaliana, increasing or generating the activity of a gene product plastidic with the activity of a “aromatic amino acid aminotransferase”, preferably being encoded by a gene comprising the nucleic acid sequence SEQ ID NO. 102015, preferably the coding region thereof, conferred the production of or the increase in tryptophan compared with the wild type control.

The nucleic acid sequence of Yjr130c from Saccharomyces cerevisiae, e.g. as shown in the respective line in column 5 of Table I, application no. 5, has been published in A. And the activity of the gene product thereof is the activity of cystathionine gamma-synthase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “cystathionine gamma-synthase”, especially from Saccharomyces cerevisiae or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said Yjr130c, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said Yjr130c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said Yjr130c, or a functional equivalent or a homolog thereof as depicted in column 8 of Table II, application no. 5, preferably a homolog or functional equivalent as depicted in column 8 of Table II B, application no. 5, and being depicted in the same respective line as said Yjr130c, and preferably the activity is increased non-targeted.

Accordingly, in one embodiment, the molecule which activity is to be increased in the process of the invention is the gene product with an activity as a “cystathionine gamma-synthase”, preferably it is the molecule of section (a) or (b) of this paragraph.

In particular, it was observed that in plants, especially in Arabidopsis thaliana, increasing or generating the activity of a gene product non-targeted with the activity of a “cystathionine gamma-synthase”, preferably being encoded by a gene comprising the nucleic acid sequence SEQ ID NO. 14843, preferably the coding region thereof, conferred the production of or the increase in tryptophan compared with the wild type control.

The nucleic acid sequence of Yjr153w from Saccharomyces cerevisiae, e.g. as shown in the respective line in column 5 of Table I, application no. 5, has been published in A. And the activity of the gene product thereof is the activity of polygalacturonase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “polygalacturonase”, especially from Saccharomyces cerevisiae or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said Yjr153w, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said Yjr153w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said Yjr153w, or a functional equivalent or a homolog thereof as depicted in column 8 of Table II, application no. 5, preferably a homolog or functional equivalent as depicted in column 8 of Table II B, application no. 5, and being depicted in the same respective line as said Yjr153w, and preferably the activity is increased non-targeted.

Accordingly, in one embodiment, the molecule which activity is to be increased in the process of the invention is the gene product with an activity as a “polygalacturonase”, preferably it is the molecule of section (a) or (b) of this paragraph.

In particular, it was observed that in plants, especially in Arabidopsis thaliana, increasing or generating the activity of a gene product non-targeted with the activity of a “polygalacturonase”, preferably being encoded by a gene comprising the nucleic acid sequence SEQ ID NO. 66274, preferably the coding region thereof, conferred the production of or the increase in tryptophan compared with the wild type control.

The nucleic acid sequence of Ykr089c from Saccharomyces cerevisiae, e.g. as shown in the respective line in column 5 of Table I, application no. 5, has been published in A. And the activity of the gene product thereof is the activity of lipase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “lipase”, especially from Saccharomyces cerevisiae or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said Ykr089c, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said Ykr089c, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said Ykr089c, or a functional equivalent or a homolog thereof as depicted in column 8 of Table II, application no. 5, preferably a homolog or functional equivalent as depicted in column 8 of Table II B, application no. 5, and being depicted in the same respective line as said Ykr089c, and preferably the activity is increased mitochondrial.

Accordingly, in one embodiment, the molecule which activity is to be increased in the process of the invention is the gene product with an activity as a “lipase”, preferably it is the molecule of section (a) or (b) of this paragraph.

In particular, it was observed that in plants, especially in Arabidopsis thaliana, increasing or generating the activity of a gene product mitochondrial with the activity of a “lipase”, preferably being encoded by a gene comprising the nucleic acid sequence SEQ ID NO. 102061, preferably the coding region thereof, conferred the production of or the increase in tryptophan compared with the wild type control.

The nucleic acid sequence of Ynl101w from Saccharomyces cerevisiae, e.g. as shown in the respective line in column 5 of Table I, application no. 5, has been published in A. And the activity of the gene product thereof is the activity of amino acid transporter.

Accordingly, in one embodiment, the process of the present invention for producing tryptophan in a non-human organism, like a microorganism or a plant or a part thereof, comprises increasing or generating the activity of a gene product with the activity of a gene product conferring the activity of “amino acid transporter”, especially from Saccharomyces cerevisiae or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said Ynl101w, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said Ynl101w, and preferably the activity is increased plastidic, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said Ynl101w, or a functional equivalent or a homolog thereof as depicted in column 8 of Table II, application no. 5, preferably a homolog or functional equivalent as depicted in column 8 of Table II B, application no. 5, and being depicted in the same respective line as said Ynl101w, and preferably the activity is increased plastidic.

Accordingly, in one embodiment, the molecule which activity is to be increased in the process of the invention is the gene product with an activity as a “amino acid transporter”, preferably it is the molecule of section (a) or (b) of this paragraph.

In particular, it was observed that in plants, especially in Arabidopsis thaliana, increasing or generating the activity of a gene product plastidic with the activity of a “amino acid transporter”, preferably being encoded by a gene comprising the nucleic acid sequence SEQ ID NO. 92339, preferably the coding region thereof, conferred the production of or the increase in tryptophan compared with the wild type control.

The nucleic acid sequence of Yor244w from Saccharomyces cerevisiae, e.g. as shown in the respective line in column 5 of Table I, application no. 5, has been published in A. And the activity of the gene product thereof is the activity of histone acetyltransferase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “histone acetyltransferase”, especially from Saccharomyces cerevisiae or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said Yor244w, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said Yor244w, and preferably the activity is increased plastidic, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said Yor244w, or a functional equivalent or a homolog thereof as depicted in column 8 of Table II, application no. 5, preferably a homolog or functional equivalent as depicted in column 8 of Table II B, application no. 5, and being depicted in the same respective line as said Yor244w, and preferably the activity is increased plastidic.

Accordingly, in one embodiment, the molecule which activity is to be increased in the process of the invention is the gene product with an activity as a “histone acetyltransferase”, preferably it is the molecule of section (a) or (b) of this paragraph.

In particular, it was observed that in plants, especially in Arabidopsis thaliana, increasing or generating the activity of a gene product plastidic with the activity of a “histone acetyltransferase”, preferably being encoded by a gene comprising the nucleic acid sequence SEQ ID NO. 92540, preferably the coding region thereof, conferred the production of or the increase in tryptophan compared with the wild type control.

The nucleic acid sequence of Ypl117c from Saccharomyces cerevisiae, e.g. as shown in the respective line in column 5 of Table I, application no. 5, has been published in A. And the activity of the gene product thereof is the activity of isopentenyl diphosphate isomerase.

Accordingly, in one embodiment, the process of the present invention for producing tryptophan in a non-human organism, like a microorganism or a plant or a part thereof, comprises increasing or generating the activity of a gene product with the activity of a gene product conferring the activity of “isopentenyl diphosphate isomerase”, especially from Saccharomyces cerevisiae or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said Ypl117c, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said Ypl117c, and preferably the activity is increased plastidic, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said Ypl117c, or a functional equivalent or a homolog thereof as depicted in column 8 of Table II, application no. 5, preferably a homolog or functional equivalent as depicted in column 8 of Table II B, application no. 5, and being depicted in the same respective line as said Ypl117c, and preferably the activity is increased plastidic.

Accordingly, in one embodiment, the molecule which activity is to be increased in the process of the invention is the gene product with an activity as a “isopentenyl diphosphate isomerase”, preferably it is the molecule of section (a) or (b) of this paragraph.

In particular, it was observed that in plants, especially in Arabidopsis thaliana, increasing or generating the activity of a gene product plastidic with the activity of a “isopentenyl diphosphate isomerase”, preferably being encoded by a gene comprising the nucleic acid sequence SEQ ID NO. 67998, preferably the coding region thereof, conferred the production of or the increase in tryptophan compared with the wild type control.

The nucleic acid sequence of Ypr119w from Saccharomyces cerevisiae, e.g. as shown in the respective line in column 5 of Table I, application no. 5, has been published in A. And the activity of the gene product thereof is the activity of cyclin.

Accordingly, in one embodiment, the process of the present invention for producing tryptophan in a non-human organism, like a microorganism or a plant or a part thereof, comprises increasing or generating the activity of a gene product with the activity of a gene product conferring the activity of “cyclin”, especially from Saccharomyces cerevisiae or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said Ypr119w, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said Ypr119w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said Ypr119w, or a functional equivalent or a homolog thereof as depicted in column 8 of Table II, application no. 5, preferably a homolog or functional equivalent as depicted in column 8 of Table II B, application no. 5, and being depicted in the same respective line as said Ypr119w, and preferably the activity is increased non-targeted.

Accordingly, in one embodiment, the molecule which activity is to be increased in the process of the invention is the gene product with an activity as a “cyclin”, preferably it is the molecule of section (a) or (b) of this paragraph.

In particular, it was observed that in plants, especially in Arabidopsis thaliana, increasing or generating the activity of a gene product non-targeted with the activity of a “cyclin”, preferably being encoded by a gene comprising the nucleic acid sequence SEQ ID NO. 98445, preferably the coding region thereof, conferred the production of or the increase in tryptophan compared with the wild type control.

The nucleic acid sequence of Zm_{—}4842_BE510522 from Zea mays, e.g. as shown in the respective line in column 5 of Table I, application no. 5, is unpublished. And the activity of the gene product thereof is the activity of Zm_{—}4842_BE510522-protein.

Accordingly, in one embodiment, the process of the present invention for producing tryptophan in a non-human organism, like a microorganism or a plant or a part thereof, comprises increasing or generating the activity of a gene product with the activity of a gene product conferring the activity of “Zm_{—}4842_BE510522-protein”, especially from Zea mays or its functional equivalent or its homolog, e.g. the increase of
(a) a gene product of a gene comprising the nucleic acid molecule as shown in the respective line in column 5 of Table I, application no. 5, preferably the coding region thereof, or a homolog or a fragment thereof, and being depicted in the same respective line as said Zm_{—}4842_BE510522, or a functional equivalent or a homolog thereof as shown in column 8 of Table I, application no. 5, preferably the coding region thereof, particularly a homolog or functional equivalent as shown in column 8 of Table I B, application no. 5, and being depicted in the same respective line as said Zm_{—}4842_BE510522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 5, respectively, and being depicted in the same respective line as said Zm_{—}4842_BE510522, or a functional equivalent or a homolog thereof as depicted in column 8 of Table II, application no. 5, preferably a homolog or functional equivalent as depicted in column 8 of Table II B, application no. 5, and being depicted in the same respective line as said Zm_{—}4842_BE510522, and preferably the activity is increased non-targeted.

Accordingly, in one embodiment, the molecule which activity is to be increased in the process of the invention is the gene product with an activity as a “Zm_{—}4842_BE510522-protein”, preferably it is the molecule of section (a) or (b) of this paragraph.

In particular, it was observed that in plants, especially in Arabidopsis thaliana, increasing or generating the activity of a gene product non-targeted with the activity of a “Zm_{—}4842_BE510522-protein”, preferably being encoded by a gene comprising the nucleic acid sequence SEQ ID NO. 68413, preferably the coding region thereof, conferred the production of or the increase in tryptophan compared with the wild type control.

[0105.1.1.5] to [0107.1.1.5] for the disclosure of these paragraphs see [0105.1.1.1] to [0107.1.1.1] above.

A protein having an activity conferring an increase in the amount or level of the fine chemical tryptophan, upon targeting to the plastids or mitochondria or upon non-targeting, preferably has the structure of the respective polypeptide described herein, in particular of the polypeptides comprising the consensus sequence or at least one polypeptide motifs as shown in the respective line in Table IV, application no. 5, column 8 or of the polypeptide comprising an amino acid sequence as disclosed in the respective line in Table II, application no. 5, columns 5 or 8, or homologs or fragments thereof as described herein, or is encoded by the nucleic acid molecule characterized herein or the nucleic acid molecule according to the invention, for example by the nucleic acid molecule as shown in the respective line in Table I, application no. 5, columns 5 or 8, preferably the coding region thereof, or homologs or fragments thereof and has the herein mentioned activity.

[0109.1.1.5] to [0110.1.1.5] for the disclosure of these paragraphs see [0109.1.1.1] to [0110.1.1.1] above.

In one embodiment, the process of the present invention comprises one or more of the following steps
(a) stabilizing a protein conferring the generation or increased expression of a protein encoded by the nucleic acid molecule of the invention or of the polypeptide of the invention, e.g. of a polypeptide having the activity selected from the group consisting of 3-deoxy-7-phosphoheptulonate synthase, 3-ketoacyl-CoA thiolase, acetyltransferase, aldehyde dehydrogenase, amino acid transporter, aromatic amino acid aminotransferase, At1g19800 protein, At2g33510-protein, At4g32480-protein, At5g16650-protein, at5g21910-protein, ATP synthase subunit E, ATP-binding component of a transport system, auxin response factor, b1163-protein, b1346-protein, b1522-protein, b1796-protein, b2739-protein, b2849-protein, b3989-protein, b4029-protein, b4121-protein, binding-protein-dependent transport systems inner membrane component, branched-chain amino acid permease, calcium-dependent protein kinase, carbon dioxide concentrating mechanism protein, cardiolipin synthase, CBL-interacting protein kinase, Chaperone protein CIpB, choline kinase, coproporphyrinogen III oxidase, cullin, cyclin, cyclin D , cystathionine gammasynthase, DNA mismatch repair protein, electron transfer flavoprotein subunit beta, elongation factor Tu, enoyl-CoA hydratase, eukaryotic translation initiation factor, eukaryotic translation initiation factor 5, fimbrial-like protein, flavodoxin, geranylgeranyl pyrophosphate synthase, gibberellin 20-oxidase, glucose dehydrogenase, glucose-6-phosphate 1-dehydrogenase, glutamate-ammonia-ligase, glutamine synthetase, glutaredoxin, glycerol kinase, glycosyl transferase, harpin-induced family protein, histone acetyltransferase, hydrolase, imidazole glycerol phosphate synthase subunit, isochorismate synthase, isopentenyl diphosphate isomerase, Leucyl/phenylalanyl-tRNA-protein transferase, lipase, L-ribulose-5-phosphate 4-epimerase, malate dehydrogenase, malic enzyme, methylaccepting chemotaxis protein, methyltransferase, monthiol glutaredoxin, NAD(P)H-quinone oxidoreductase subunit, nitrate/nitrite transport protein, photosystem II protein, polygalacturonase, precorrin methylase, protein kinase, protein phosphatase, putative transport system permease protein, pyruvate kinase, recombinase A, RNA binding protein, RNA-binding protein, Sec-independent protein translocase subunit, short-chain alcohol dehydrogenase family, slr1107-protein, threonine synthase, transaldolase, transcription factor, transcriptional regulator, transport protein, trehalose-phosphatase, tryptophan biosynthesis protein, yfl054c-protein, and Zm_{—}4842_BE510522-protein, or of a polypeptide as indicated in the respective line in Table II, application no. 5, columns 5 or 8, or its homologs or fragments, and conferring the production of or an increase in tryptophan, respectively; and/or
(b) stabilizing a mRNA conferring the generation or increased expression of a FCRP, e.g. protein encoded by the nucleic acid molecule of the invention or its homologs or fragments, or of a mRNA encoding the polypeptide of the present invention having the hereinmentioned activity selected from the group consisting of said activities mentioned in (a) and conferring the production of or an increase in tryptophan, respectively; and/or
(c) increasing the specific activity of a protein conferring the increased expression of a FCRP, e.g. a protein encoded by the nucleic acid molecule of the invention or of the polypeptide of the present invention having herein-mentioned tryptophan generating or increasing activity, respectively, e.g. of a polypeptide having the activity of a protein as indicated in the respective line in Table II, application no. 5, columns 5 or 8, or its homologs or fragments, or decreasing the inhibitory regulation of the polypeptide of the invention; and/or
(d) generating or increasing the expression of an endogenous or artificial transcription factor mediating the expression of a protein conferring the generation or increased expression of a FCRP, e.g. a protein encoded by the nucleic acid molecule of the invention or of the polypeptide of the invention having herein-mentioned activity selected from the group consisting of said activities mentioned in (a) and conferring a tryptophan increasing activity, respectively, e.g. of a polypeptide having the activity of a protein as indicated in the respective line in Table II, application no. 5, columns 5 or 8 or its homologs; and/or
(e) stimulating activity of a protein conferring the increased expression of a FCRP, e.g. a protein encoded by the nucleic acid molecule of the present invention or a polypeptide of the present invention having herein-mentioned activity selected from the group consisting of said activities mentioned in (a) and conferring a tryptophan increasing activity, respectively, e.g. of a polypeptide having the activity of a protein as indicated in the respective line in Table II, application no. 5, columns 5 or 8, or its homologs or fragments, by adding one or more exogenous inducing factors to the non-human organism or parts thereof; and/or
(f) expressing a transgenic gene encoding a protein conferring the increased expression of a FCRP, e.g. a polypeptide encoded by the nucleic acid molecule of the present invention or a polypeptide of the present invention, having herein-mentioned activity selected from the group consisting of said activities mentioned in (a) and conferring a tryptophan increasing activity, respectively, e.g. of a polypeptide having the activity of a protein as indicated in the respective line in Table II, application no. 5, columns 5 or 8, or its homologs or fragments, and/or
(g) increasing the copy number of a gene conferring the increased expression of a nucleic acid molecule encoding a FCRP, e.g. a polypeptide encoded by the nucleic acid molecule of the invention or the polypeptide of the invention having herein-mentioned activity selected from the group consisting of said activities mentioned in (a) and conferring a tryptophan increasing activity, respectively, e.g. of a polypeptide having the activity of a protein as indicated in the respective line in Table II, application no. 5, columns 5 or 8, or its homologs or fragments; and/or
(h) increasing the expression of the endogenous gene encoding the FCRP, polypeptide of the invention, e.g. a polypeptide encoded by the nucleic acid molecule of the invention or the polypeptide of the invention having herein-mentioned activity selected from the group consisting of said activities mentioned in (a) and conferring a tryptophan; increasing activity, respectively, e.g. of a polypeptide having the activity of a protein as indicated in the respective line in Table II, application no. 5, columns 5 or 8, or its homologs or fragments, by adding positive expression or removing negative expression elements, e.g. homologous recombination can be used to either introduce positive regulatory elements like for plants the 35S enhancer into the promoter or to remove repressor elements form regulatory regions. Further gene conversion methods can be used to disrupt repressor elements or to enhance to activity of positive elements. Positive elements can be randomly introduced in plants by T-DNA or transposon mutagenesis and lines can be identified in which the positive elements have be integrated near to a gene of the invention, the expression of which is thereby enhanced; and/or
(i) modulating growth conditions of the non-human organism in such a manner, that the expression or activity of the gene encoding the FCRP, e.g. a protein of the invention or the protein itself is enhanced, for example microorganisms or plants can be grown for example under a higher temperature regime leading to an enhanced expression of heat shock proteins, which can lead to an enhanced tryptophan production, respectively; and/or
(j) selecting of non-human organisms with especially high activity of the proteins of the invention from natural or from mutagenized resources and breeding them into the target organisms, e.g. the elite crops; and/or
(k) directing a protein encoded by the nucleic acid molecule of the invention or of the polypeptide of the present invention having herein-mentioned activity selected from the group consisting of said activities mentioned in (a) and conferring a tryptophan increasing activity, respectively, e.g. of a polypeptide having the activity of a protein as indicated in the respective line in Table II, application no. 5, columns 5 or 8, or its homologs or fragments, if for the corresponding nucleic acid molecule in the respective line in column 6 of Table I the term “plastidic” is indicated, to the plastids by the addition of a plastidial targeting sequence or if for the corresponding nucleic acid molecule in the respective line in column 6 of Table I the term “mitochondrial” is indicated, to the mitochondria by the addition of a mitochondrial targeting sequence; and/or
(l) generating the expression of a protein encoded by the nucleic acid molecule of the invention or of the polypeptide of the present invention having herein-mentioned activity selected from the group consisting of said activities mentioned in (a) and conferring a tryptophan increasing activity, respectively, e.g. of a polypeptide having the activity of a protein as indicated in the respective line in Table II, application no. 5, columns 5 or 8, or its homologs or fragments, if for the corresponding nucleic acid molecule in the respective line in column 6 of Table I the term “plastidic” or “mitochondrial” is indicated, in these organelles by the stable or transient transformation, advantageously stable transformation, of organelles, preferably plastids or mitochondria, with an inventive nucleic acid sequence preferably in form of an expression cassette containing said sequence leading to the expression of the nucleic acids or polypeptides of the invention in the respective organelle; and/or
(m) generating the expression of a protein encoded by the nucleic acid molecule of the invention or of the polypeptide of the present invention having herein-mentioned activity selected from the group consisting of said activities mentioned in (a) and conferring a tryptophan increasing activity, respectively, e.g. of a polypeptide having the activity of a protein as indicated in the respective line in Table II, application no. 5, columns 5 or 8, or its homologs or fragments, if for the corresponding nucleic acid molecule in the respective line in column 6 of Table I the term “plastidic” or “mitochondrial” is indicated, in these organelles by integration of a nucleic acid of the invention into the genome of the respective organelle under control of preferable a promoter selective for the respective organelle.

Preferably, said mRNA is the coding region of the nucleic acid molecule of the present invention and/or the protein conferring the increased expression of a protein encoded by the coding region of the nucleic acid molecule of the present invention alone or linked to a transit nucleic acid sequence or transit peptide encoding nucleic acid sequence or the polypeptide having the herein mentioned activity, e.g. conferring the generation of or increase of tryptophan, respectively, after increasing the expression or activity of the encoded polypeptide, non-targeted or in organelles such as plastids and/or mitochondria, preferably plastids, or having the activity of a polypeptide having an activity as the protein as shown in the respective line in Table II, application no. 5, column 3, or its homologs. Preferably the increase of tryptophan, respectively, takes place non-targeted or in plastids and/or mitochondria, preferably non-targeted or in plastids.

[0113.1.1.5] to [0122.1.1.5] for the disclosure of these paragraphs see [0113.1.1.1] to [0122.1.1.1] above.

The activation of an endogenous polypeptide having above-mentioned activity, e.g. having the activity of a protein as shown in the respective line in Table II, application no. 5, column 3 or of the polypeptide of the invention, e.g. conferring the increase of the fine chemical tryptophan, respectively, by increase of expression or activity in the cytoplasm, and/or in the cytosol, and/or in an organelle, such as plastids or mitochondria, can also be increased by introducing a synthetic transcription factor, which binds close to the coding region of the gene encoding the protein as shown in the respective line in Table II, application no. 5, column 5 or 8, or homologs or fragments thereof, and activates its transcription. A chimeric zinc finger protein can be constructed, which comprises a specific DNA-binding domain and an activation domain as e.g. the VP16 domain of Herpes Simplex virus. The specific binding domain can bind to the regulatory region of the gene encoding the protein as shown in the respective line in Table II, application no. 5, column 5 or 8, or homologs or fragments thereof. The expression of the chimeric transcription factor in a non-human organism, in particular in a plant, leads to a specific expression of the protein as shown in the respective line in Table II, application no. 5, column 5 or 8, or homologs or fragments thereof, see e.g. in WO01/52620, Oriz, Proc. Natl. Acad. Sci. USA 99, 13290 (2002), or Guan, Proc. Natl. Acad. Sci. USA 99, 13296 (2002).

[0124.1.1.5] to [0127.1.1.5] for the disclosure of these paragraphs see [0124.1.1.1] to [0127.1.1.1] above.

Owing to the introduction of a gene or a plurality of genes conferring the expression of the nucleic acid molecule of the invention or the polypeptide of the invention, for example the nucleic acid construct mentioned below, or encoding the protein as shown in the respective line in Table II, application no. 5, column 5 or 8, or homologs or fragments thereof, into a non-human organism alone or in combination with other genes, it is possible not only to increase the biosynthetic flux towards the end product, but also to increase, modify or create de novo an advantageous, preferably novel metabolites composition in the non-human organism, e.g. an advantageous composition comprising a higher content of (from a viewpoint of nutritional physiology limited) tryptophan and if desired other amino acids, and/or other metabolites, in free or bound form.

for the disclosure of this paragraph see [0129.1.1.1] above [0130.1.5.5]

Accordingly, in one embodiment, the process according to the invention relates to a process, which comprises:
(a) providing a non-human organism, preferably a microorganism, a plant cell, a plant tissue, a plant or a part thereof, more preferably a microorganism, a plant tissue, a plant or a part thereof;
(b) increasing the activity of a protein as shown in the respective line in Table II, application no. 5, column 3 or of a polypeptide being encoded by the respective nucleic acid molecule of the present invention and described below, e.g. conferring an increase of the fine chemical tryptophan, respectively, in the non-human organism, preferably in the microorganism, the plant cell, the plant tissue, the plant or a part thereof, more preferably a microorganism, a plant tissue, a plant or a part thereof, especially cytoplasmic or in an organelle, like plastids or mitochondria,
(c) growing the non-human organism, preferably the microorganism, the plant cell, the plant tissue, the plant or a part thereof, under conditions which permit the production of said fine chemical in the non-human organism, preferably the microorganism, the plant cell, the plant tissue, the plant or a part thereof; and
(d) if desired, recovering, optionally isolating, said fine chemical, in free and/or bound form, and, optionally further free and/or bound amino acids, and/or other metabolites synthesized by the non-human organism, the microorganism, the plant cell, the plant tissue, the plant or a part thereof.

[0131.1.1.5] to [0139.1.1.5] for the disclosure of these paragraphs see [0131.1.1.1] to [0139.1.1.1] above.

In another embodiment, the invention relates to a (isolated) nucleic acid molecule, which comprises a nucleic acid molecule selected from the group consisting of:
(a) a nucleic acid molecule encoding a polypeptide shown in Table II, application no. 5, column 5 or 8, preferably shown in Table II B, application no. 5, column 8, or a homolog or a fragment thereof;
(b) a nucleic acid molecule shown in Table I, application no. 5, column 5 or 8, preferably shown in Table I B, application no. 5, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 5, preferably in column 8 of Table II B, application no. 5;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 5, preferably shown in column 8 of Table I B, application no. 5, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 5;
(i) a nucleic acid molecule encoding a polypeptide having the activity represented by a respective protein as depicted in column 5 of Table II, application no. 5;
(j) a nucleic acid molecule comprising a polynucleotide which is obtained by amplifying a cDNA library or a genomic library using the primers in Table III, application no. 5, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 5.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II application no. 5 and confers a generation or an increase of the respective fine chemical in a non-human organism or a part thereof as compared to a corresponding wild-type non-human organism or part thereof.

In another embodiment, the invention relates to a (isolated) nucleic acid molecule, which comprises a nucleic acid molecule selected from the group consisting of:
(a) a nucleic acid molecule encoding a polypeptide shown in Table II, column 5 or 8, application no. 5, preferably shown in Table II A, application no. 5, in column 5 or in Table II A, application no. 5, column 8 or in Table II B, application no. 5, column 8, or a homolog or a fragment thereof;
(b) a nucleic acid molecule shown in Table I, application no. 5, column 5 or 8, preferably shown in Table I A, application no. 5, in column 5 or in Table I A, application no. 5, column 8 or in Table I B, application no. 5, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 5, preferably shown in Table II A, application no. 5, in column 5 or in Table II A, application no. 5, column 8 or in Table II B, application no. 5, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 5, preferably shown in Table I A, application no. 5, in column 5 or in Table I A, application no. 5, column 8 or in Table I B column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 5;
(i) a nucleic acid molecule encoding a polypeptide having the activity represented by a respective protein as depicted in column 5 of Table II, application no. 5;
(j) a nucleic acid molecule comprising a polynucleotide which is obtained by amplifying a cDNA library or a genomic library using the primers in Table III, application no. 5, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt,500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto;
   whereby, preferably, the nucleic acid molecule according to (a), (b), (c), (d), (e), (f), (g), (h), (i), (j) or (k) distinguishes over the sequence depicted in Table I A and/or I B, application no. 5, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 5, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 5, column 5 or 8, or the coding region thereof, respectively. In a further embodiment the nucleic acid molecule does not encode the polypeptide sequence shown in Table II A and/or II B, application no. 5, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 5, column 5 or 8 and does not encode a protein of the sequence shown in Table II A and/or II B, application no. 5, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 5, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 5, column 5 or 8 but less than 100%, preferably less than 99.999%, 99.99%, 99.9%, 99%, 98%, 97%, 96% or 95% identical to the sequence shown in Table II A and/or II B, application no. 5, column 5 or 8.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 5.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II, application no. 5, and confers a generation or an increase of the respective fine chemical in a non-human organism or a part thereof as compared to a corresponding wild-type non-human organism or part thereof.

[0144.1.1.5] to [0155.1.1.5] for the disclosure of these paragraphs see [0144.1.1.1] to [0155.1.1.1] above.

In a preferred embodiment a nucleic acid construct, for example an expression cassette, comprises upstream, i.e. at the 5′ end of the encoding sequence, a promoter and downstream, i.e. at the 3′ end, a polyadenylation signal and optionally other regulatory elements which are operably linked to the intervening encoding sequence with one of the nucleic acids as depicted in the respective line in Table I, application no. 5, column 5 or 8, preferably the coding region thereof, or a homolog or a fragment thereof. By an operable linkage is meant the sequential arrangement of promoter, encoding sequence, terminator, optionally other regulatory elements and optionally targeting sequences in such a way that each of the regulatory elements/targeting sequence can fulfill its function in the expression of the encoding sequence in due manner. In one embodiment the sequences preferred for operable linkage are targeting sequences for ensuring subcellular localization in organelles, like plastids or mitochondria. However, targeting sequences for ensuring subcellular localization in the endoplasmic reticulum (=ER), in the nucleus, in oil corpuscles or other compartments may also be employed as well as translation promoters such as the 5′ lead sequence in tobacco mosaic virus (Gallie et al., Nucl. Acids Res. 15, 8693 (1987)).

for the disclosure of this paragraph see [0157.1.1.1] above [0158.1.5.5] In an embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 5, column 5 or 8, preferably shown in Table II A, application no. 5, column 5, or in Table II A, application no. 5, column 8, or in Table II B, application no. 5, column 8, or a homolog or a fragment thereof;
   (b) a nucleic acid molecule shown in Table I, application no. 5, column 5 or 8, preferably shown in Table I A, application no. 5, column 5, or in Table I A, application no. 5, column 8, or in Table I B, application no. 5, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 5, preferably shown in Table II A, application no. 5, column 5, or in Table II A, application no. 5, column 8, or in Table II B, application no. 5, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 5, preferably shown in Table I A, application no. 5, column 5, or in Table I A, application no. 5, column 8, or in Table I B, application no. 5, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 5;
   (i) a nucleic acid molecule encoding a polypeptide having the activity represented by a respective protein as depicted in column 5 of Table II, application no. 5;
   (j) a nucleic acid molecule comprising a polynucleotide which is obtained by amplifying a cDNA library or a genomic library using the primers in Table III, application no. 5, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto.
      and, optionally
3) nucleic acid molecule encoding a transit peptide, preferably encoding a plastidal transit peptide or a mitochondrial transit peptide;
   which are operable linked.
   (All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 5.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 5 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 5, in the same line as of the respective nucleic acid molecule according to 2) “plastidic” is depicted, or encoding a mitochondrial transit peptide, especially in case in column 6 of Table I, application no. 5, in the same line as of the respective nucleic acid molecule according to 2) “mitochondrial” is depicted.

In another embodiment thereof the expression cassette does not comprise 3) a nucleic acid molecule encoding a transit peptide, especially in case in column 6 of Table I, application no. 5, in the same line as of the respective nucleic acid molecule according to 2) “non-targeted” is depicted.

In a further embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 5, column 5 or 8, preferably shown in Table II A, application no. 5, column 5, or in Table II A, application no. 5, column 8, or in Table II B, application no. 5, column 8, or a homolog or a fragment thereof;
   (b) a nucleic acid molecule shown in Table I, application no. 5, column 5 or 8, preferably shown in Table I A, application no. 5, column 5, or in Table I A, application no. 5, column 8, or in Table I B, application no. 5, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 5, preferably shown in Table II A, application no. 5, column 5, or in Table II A, application no. 5, column 8, or in Table II B, application no. 5, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 5, preferably shown in Table I A, application no. 5, column 5, or in Table I A, application no. 5, column 8,or in Table I B, application no. 5, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 5;
   (i) a nucleic acid molecule encoding a polypeptide having the activity represented by a respective protein as depicted in column 5 of Table II, application no. 5;
   (j) a nucleic acid molecule comprising a polynucleotide which is obtained by amplifying a cDNA library or a genomic library using the primers in Table III, application no. 5, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, optionally
3) nucleic acid molecule encoding a transit peptide, preferably encoding a plastidal transit peptide or a mitochondrial transit peptide;
   which are operable linked and
   whereby, preferably, the nucleic acid molecule according to 2 (a), (b), (c), (d), (e), (f), (g), (h), (i), (j) or (k) distinguishes over the sequence depicted in Table I A and/or I B, application no. 5, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention according to 2) does not consist of the sequence shown in Table I A and/or I B, application no. 5, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 5, column 5 or 8, or the coding region thereof, respectively. In a further embodiment the nucleic acid molecule does not encode the polypeptide sequence shown in Table II A and/or II B, application no. 5, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 5, column 5 or 8 and does not encode a protein of the sequence shown in Table II A and/or II B, application no. 5, column 5 or 8. Accordingly, in one embodiment, the protein encoded by a sequence of a nucleic acid according to 2(a), (b), (c), (d), (e), (f), (g), (h), (i), (j) or (k) does not consist of the sequence shown in Table I A and/or I B, application no. 5, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 5, column 5 or 8 but less than 100%, 99.999%, 99.99%, 99.9%, 99%, 98%, 97%, 96% or 95% identical to the sequence shown in Table II A and/or II B, application no. 5, column 5 or 8.
   (All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 5.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 5 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 5, in the same line as of the respective nucleic acid molecule according to 2) “plastidic” is depicted, or encoding a mitochondrial transit peptide, especially in case in column 6 of Table I, application no. 5, in the same line as of the respective nucleic acid molecule according to 2) “mitochondrial” is depicted.

In another embodiment thereof the expression cassette does not comprise 3) a nucleic acid molecule encoding a transit peptide, especially in case in column 6 of Table I, application no. 5, in the same line as of the respective nucleic acid molecule according to 2) “non-targeted” is depicted.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 16883, or a homolog or fragment thereof, or a homolog thereof being depicted in Table I, application no. 5, column 8, in the same line as SEQ ID NO. 16883, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 16883 is depicted, plastidic is mentioned, or a mitochondrial transit peptide in case in Table I, application no. 5, in column 6 in the same line as SEQ ID NO. 16883 is depicted, mitochondrial is mentioned; which is operable linked, too.

In another embodiment thereof the expression cassette does not comprise an additional transit peptide in case in Table I, application no. 5, in column 6 in the same line as SEQ ID NO. 16883 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17637, or a homolog or fragment thereof, or a homolog thereof being depicted in Table I, application no. 5, column 8, in the same line as SEQ ID NO. 17637, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 17637 is depicted, plastidic is mentioned, or a mitochondrial transit peptide in case in Table I, application no. 5, in column 6 in the same line as SEQ ID NO. 17637 is depicted, mitochondrial is mentioned; which is operable linked, too.

In another embodiment thereof the expression cassette does not comprise an additional transit peptide in case in Table I, application no. 5, in column 6 in the same line as SEQ ID NO. 17637 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17701, or a homolog or fragment thereof, or a homolog thereof being depicted in Table I, application no. 5, column 8, in the same line as SEQ ID NO. 17701, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 17701 is depicted, plastidic is mentioned, or a mitochondrial transit peptide in case in Table I, application no. 5, in column 6 in the same line as SEQ ID NO. 17701 is depicted, mitochondrial is mentioned; which is operable linked, too.

In another embodiment thereof the expression cassette does not comprise an additional transit peptide in case in Table I, application no. 5, in column 6 in the same line as SEQ ID NO. 17701 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17968, or a homolog or fragment thereof, or a homolog thereof being depicted in Table I, application no. 5, column 8, in the same line as SEQ ID NO. 17968, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 17968 is depicted, plastidic is mentioned, or a mitochondrial transit peptide in case in Table I, application no. 5, in column 6 in the same line as SEQ ID NO. 17968 is depicted, mitochondrial is mentioned; which is operable linked, too.

In another embodiment thereof the expression cassette does not comprise an additional transit peptide in case in Table I, application no. 5, in column 6 in the same line as SEQ ID NO. 17968 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 18235, or a homolog or fragment thereof, or a homolog thereof being depicted in Table I, application no. 5, column 8, in the same line as SEQ ID NO. 18235, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 18235 is depicted, plastidic is mentioned, or a mitochondrial transit peptide in case in Table I, application no. 5, in column 6 in the same line as SEQ ID NO. 18235 is depicted, mitochondrial is mentioned; which is operable linked, too.

In another embodiment thereof the expression cassette does not comprise an additional transit peptide in case in Table I, application no. 5, in column 6 in the same line as SEQ ID NO. 18235 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 19502, or a homolog or fragment thereof, or a homolog thereof being depicted in Table I, application no. 5, column 8, in the same line as SEQ ID NO. 19502, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 19502 is depicted, plastidic is mentioned, or a mitochondrial transit peptide in case in Table I, application no. 5, in column 6 in the same line as SEQ ID NO. 19502 is depicted, mitochondrial is mentioned; which is operable linked, too.

In another embodiment thereof the expression cassette does not comprise an additional transit peptide in case in Table I, application no. 5, in column 6 in the same line as SEQ ID NO. 19502 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 19874, or a homolog or fragment thereof, or a homolog thereof being depicted in Table I, application no. 5, column 8, in the same line as SEQ ID NO. 19874, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 19874 is depicted, plastidic is mentioned, or a mitochondrial transit peptide in case in Table I, application no. 5, in column 6 in the same line as SEQ ID NO. 19874 is depicted, mitochondrial is mentioned; which is operable linked, too.

In another embodiment thereof the expression cassette does not comprise an additional transit peptide in case in Table I, application no. 5, in column 6 in the same line as SEQ ID NO. 19874 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 19919, or a homolog or fragment thereof, or a homolog thereof being depicted in Table I, application no. 5, column 8, in the same line as SEQ ID NO. 19919, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 19919 is depicted, plastidic is mentioned, or a mitochondrial transit peptide in case in Table I, application no. 5, in column 6 in the same line as SEQ ID NO. 19919 is depicted, mitochondrial is mentioned; which is operable linked, too.

In another embodiment thereof the expression cassette does not comprise an additional transit peptide in case in Table I, application no. 5, in column 6 in the same line as SEQ ID NO. 19919 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 20346, or a homolog or fragment thereof, or a homolog thereof being depicted in Table I, application no. 5, column 8, in the same line as SEQ ID NO. 20346, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 20346 is depicted, plastidic is mentioned, or a mitochondrial transit peptide in case in Table I, application no. 5, in column 6 in the same line as SEQ ID NO. 20346 is depicted, mitochondrial is mentioned; which is operable linked, too.

In another embodiment thereof the expression cassette does not comprise an additional transit peptide in case in Table I, application no. 5, in column 6 in the same line as SEQ ID NO. 20346 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 98522, or a homolog or fragment thereof, or a homolog thereof being depicted in Table I, application no. 5, column 8, in the same line as SEQ ID NO. 98522, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 98522 is depicted, plastidic is mentioned, or a mitochondrial transit peptide in case in Table I, application no. 5, in column 6 in the same line as SEQ ID NO. 98522 is depicted, mitochondrial is mentioned; which is operable linked, too.

In another embodiment thereof the expression cassette does not comprise an additional transit peptide in case in Table I, application no. 5, in column 6 in the same line as SEQ ID NO. 98522 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 21159, or a homolog or fragment thereof, or a homolog thereof being depicted in Table I, application no. 5, column 8, in the same line as SEQ ID NO. 21159, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 21159 is depicted, plastidic is mentioned, or a mitochondrial transit peptide in case in Table I, application no. 5, in column 6 in the same line as SEQ ID NO. 21159 is depicted, mitochondrial is mentioned; which is operable linked, too.

In another embodiment thereof the expression cassette does not comprise an additional transit peptide in case in Table I, application no. 5, in column 6 in the same line as SEQ ID NO. 21159 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 21497, or a homolog or fragment thereof, or a homolog thereof being depicted in Table I, application no. 5, column 8, in the same line as SEQ ID NO. 21497, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 21497 is depicted, plastidic is mentioned, or a mitochondrial transit peptide in case in Table I, application no. 5, in column 6 in the same line as SEQ ID NO. 21497 is depicted, mitochondrial is mentioned; which is operable linked, too.

In another embodiment thereof the expression cassette does not comprise an additional transit peptide in case in Table I, application no. 5, in column 6 in the same line as SEQ ID NO. 21497 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 22611, or a homolog or fragment thereof, or a homolog thereof being depicted in Table I, application no. 5, column 8, in the same line as SEQ ID NO. 22611, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 22611 is depicted, plastidic is mentioned, or a mitochondrial transit peptide in case in Table I, application no. 5, in column 6 in the same line as SEQ ID NO. 22611 is depicted, mitochondrial is mentioned; which is operable linked, too.

In another embodiment thereof the expression cassette does not comprise an additional transit peptide in case in Table I, application no. 5, in column 6 in the same line as SEQ ID NO. 22611 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 22832, or a homolog or fragment thereof, or a homolog thereof being depicted in Table I, application no. 5, column 8, in the same line as SEQ ID NO. 22832, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 22832 is depicted, plastidic is mentioned, or a mitochondrial transit peptide in case in Table I, application no. 5, in column 6 in the same line as SEQ ID NO. 22832 is depicted, mitochondrial is mentioned; which is operable linked, too.

In another embodiment thereof the expression cassette does not comprise an additional transit peptide in case in Table I, application no. 5, in column 6 in the same line as SEQ ID NO. 22832 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 1815, or a homolog or fragment thereof, or a homolog thereof being depicted in Table I, application no. 5, column 8, in the same line as SEQ ID NO. 1815, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 1815 is depicted, plastidic is mentioned, or a mitochondrial transit peptide in case in Table I, application no. 5, in column 6 in the same line as SEQ ID NO. 1815 is depicted, mitochondrial is mentioned; which is operable linked, too.

In another embodiment thereof the expression cassette does not comprise an additional transit peptide in case in Table I, application no. 5, in column 6 in the same line as SEQ ID NO. 1815 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68727, or a homolog or fragment thereof, or a homolog thereof being depicted in Table I, application no. 5, column 8, in the same line as SEQ ID NO. 68727, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 68727 is depicted, plastidic is mentioned, or a mitochondrial transit peptide in case in Table I, application no. 5, in column 6 in the same line as SEQ ID NO. 68727 is depicted, mitochondrial is mentioned; which is operable linked, too.

In another embodiment thereof the expression cassette does not comprise an additional transit peptide in case in Table I, application no. 5, in column 6 in the same line as SEQ ID NO. 68727 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68777, or a homolog or fragment thereof, or a homolog thereof being depicted in Table I, application no. 5, column 8, in the same line as SEQ ID NO. 68777, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 68777 is depicted, plastidic is mentioned, or a mitochondrial transit peptide in case in Table I, application no. 5, in column 6 in the same line as SEQ ID NO. 68777 is depicted, mitochondrial is mentioned; which is operable linked, too.

In another embodiment thereof the expression cassette does not comprise an additional transit peptide in case in Table I, application no. 5, in column 6 in the same line as SEQ ID NO. 68777 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 2367, or a homolog or fragment thereof, or a homolog thereof being depicted in Table I, application no. 5, column 8, in the same line as SEQ ID NO. 2367, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 2367 is depicted, plastidic is mentioned, or a mitochondrial transit peptide in case in Table I, application no. 5, in column 6 in the same line as SEQ ID NO. 2367 is depicted, mitochondrial is mentioned; which is operable linked, too.

In another embodiment thereof the expression cassette does not comprise an additional transit peptide in case in Table I, application no. 5, in column 6 in the same line as SEQ ID NO. 2367 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 2573, or a homolog or fragment thereof, or a homolog thereof being depicted in Table I, application no. 5, column 8, in the same line as SEQ ID NO. 2573, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 2573 is depicted, plastidic is mentioned, or a mitochondrial transit peptide in case in Table I, application no. 5, in column 6 in the same line as SEQ ID NO. 2573 is depicted, mitochondrial is mentioned; which is operable linked, too.

In another embodiment thereof the expression cassette does not comprise an additional transit peptide in case in Table I, application no. 5, in column 6 in the same line as SEQ ID NO. 2573 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 2935, or a homolog or fragment thereof, or a homolog thereof being depicted in Table I, application no. 5, column 8, in the same line as SEQ ID NO. 2935, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 2935 is depicted, plastidic is mentioned, or a mitochondrial transit peptide in case in Table I, application no. 5, in column 6 in the same line as SEQ ID NO. 2935 is depicted, mitochondrial is mentioned; which is operable linked, too.

In another embodiment thereof the expression cassette does not comprise an additional transit peptide in case in Table I, application no. 5, in column 6 in the same line as SEQ ID NO. 2935 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4040, or a homolog or fragment thereof, or a homolog thereof being depicted in Table I, application no. 5, column 8, in the same line as SEQ ID NO. 4040, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 4040 is depicted, plastidic is mentioned, or a mitochondrial transit peptide in case in Table I, application no. 5, in column 6 in the same line as SEQ ID NO. 4040 is depicted, mitochondrial is mentioned; which is operable linked, too.

In another embodiment thereof the expression cassette does not comprise an additional transit peptide in case in Table I, application no. 5, in column 6 in the same line as SEQ ID NO. 4040 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 24311, or a homolog or fragment thereof, or a homolog thereof being depicted in Table I, application no. 5, column 8, in the same line as SEQ ID NO. 24311, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 24311 is depicted, plastidic is mentioned, or a mitochondrial transit peptide in case in Table I, application no. 5, in column 6 in the same line as SEQ ID NO. 24311 is depicted, mitochondrial is mentioned; which is operable linked, too.

In another embodiment thereof the expression cassette does not comprise an additional transit peptide in case in Table I, application no. 5, in column 6 in the same line as SEQ ID NO. 24311 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4348, or a homolog or fragment thereof, or a homolog thereof being depicted in Table I, application no. 5, column 8, in the same line as SEQ ID NO. 4348, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 4348 is depicted, plastidic is mentioned, or a mitochondrial transit peptide in case in Table I, application no. 5, in column 6 in the same line as SEQ ID NO. 4348 is depicted, mitochondrial is mentioned; which is operable linked, too.

In another embodiment thereof the expression cassette does not comprise an additional transit peptide in case in Table I, application no. 5, in column 6 in the same line as SEQ ID NO. 4348 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68987, or a homolog or fragment thereof, or a homolog thereof being depicted in Table I, application no. 5, column 8, in the same line as SEQ ID NO. 68987, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 68987 is depicted, plastidic is mentioned, or a mitochondrial transit peptide in case in Table I, application no. 5, in column 6 in the same line as SEQ ID NO. 68987 is depicted, mitochondrial is mentioned; which is operable linked, too.

In another embodiment thereof the expression cassette does not comprise an additional transit peptide in case in Table I, application no. 5, in column 6 in the same line as SEQ ID NO. 68987 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 24492, or a homolog or fragment thereof, or a homolog thereof being depicted in Table I, application no. 5, column 8, in the same line as SEQ ID NO. 24492, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 24492 is depicted, plastidic is mentioned, or a mitochondrial transit peptide in case in Table I, application no. 5, in column 6 in the same line as SEQ ID NO. 24492 is depicted, mitochondrial is mentioned; which is operable linked, too.

In another embodiment thereof the expression cassette does not comprise an additional transit peptide in case in Table I, application no. 5, in column 6 in the same line as SEQ ID NO. 24492 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 25283, or a homolog or fragment thereof, or a homolog thereof being depicted in Table I, application no. 5, column 8, in the same line as SEQ ID NO. 25283, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 25283 is depicted, plastidic is mentioned, or a mitochondrial transit peptide in case in Table I, application no. 5, in column 6 in the same line as SEQ ID NO. 25283 is depicted, mitochondrial is mentioned; which is operable linked, too.

In another embodiment thereof the expression cassette does not comprise an additional transit peptide in case in Table I, application no. 5, in column 6 in the same line as SEQ ID NO. 25283 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 69553, or a homolog or fragment thereof, or a homolog thereof being depicted in Table I, application no. 5, column 8, in the same line as SEQ ID NO. 69553, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 69553 is depicted, plastidic is mentioned, or a mitochondrial transit peptide in case in Table I, application no. 5, in column 6 in the same line as SEQ ID NO. 69553 is depicted, mitochondrial is mentioned; which is operable linked, too.

In another embodiment thereof the expression cassette does not comprise an additional transit peptide in case in Table I, application no. 5, in column 6 in the same line as SEQ ID NO. 69553 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 25428, or a homolog or fragment thereof, or a homolog thereof being depicted in Table I, application no. 5, column 8, in the same line as SEQ ID NO. 25428, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 25428 is depicted, plastidic is mentioned, or a mitochondrial transit peptide in case in Table I, application no. 5, in column 6 in the same line as SEQ ID NO. 25428 is depicted, mitochondrial is mentioned; which is operable linked, too.

In another embodiment thereof the expression cassette does not comprise an additional transit peptide in case in Table I, application no. 5, in column 6 in the same line as SEQ ID NO. 25428 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 70006, or a homolog or fragment thereof, or a homolog thereof being depicted in Table I, application no. 5, column 8, in the same line as SEQ ID NO. 70006, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 70006 is depicted, plastidic is mentioned, or a mitochondrial transit peptide in case in Table I, application no. 5, in column 6 in the same line as SEQ ID NO. 70006 is depicted, mitochondrial is mentioned; which is operable linked, too.

In another embodiment thereof the expression cassette does not comprise an additional transit peptide in case in Table I, application no. 5, in column 6 in the same line as SEQ ID NO. 70006 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 5557, or a homolog or fragment thereof, or a homolog thereof being depicted in Table I, application no. 5, column 8, in the same line as SEQ ID NO. 5557, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 5557 is depicted, plastidic is mentioned, or a mitochondrial transit peptide in case in Table I, application no. 5, in column 6 in the same line as SEQ ID NO. 5557 is depicted, mitochondrial is mentioned; which is operable linked, too.

In another embodiment thereof the expression cassette does not comprise an additional transit peptide in case in Table I, application no. 5, in column 6 in the same line as SEQ ID NO. 5557 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 27021, or a homolog or fragment thereof, or a homolog thereof being depicted in Table I, application no. 5, column 8, in the same line as SEQ ID NO. 27021, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 27021 is depicted, plastidic is mentioned, or a mitochondrial transit peptide in case in Table I, application no. 5, in column 6 in the same line as SEQ ID NO. 27021 is depicted, mitochondrial is mentioned; which is operable linked, too.

In another embodiment thereof the expression cassette does not comprise an additional transit peptide in case in Table I, application no. 5, in column 6 in the same line as SEQ ID NO. 27021 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 28738, or a homolog or fragment thereof, or a homolog thereof being depicted in Table I, application no. 5, column 8, in the same line as SEQ ID NO. 28738, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 28738 is depicted, plastidic is mentioned, or a mitochondrial transit peptide in case in Table I, application no. 5, in column 6 in the same line as SEQ ID NO. 28738 is depicted, mitochondrial is mentioned; which is operable linked, too.

In another embodiment thereof the expression cassette does not comprise an additional transit peptide in case in Table I, application no. 5, in column 6 in the same line as SEQ ID NO. 28738 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 29286, or a homolog or fragment thereof, or a homolog thereof being depicted in Table I, application no. 5, column 8, in the same line as SEQ ID NO. 29286, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 29286 is depicted, plastidic is mentioned, or a mitochondrial transit peptide in case in Table I, application no. 5, in column 6 in the same line as SEQ ID NO. 29286 is depicted, mitochondrial is mentioned; which is operable linked, too.

In another embodiment thereof the expression cassette does not comprise an additional transit peptide in case in Table I, application no. 5, in column 6 in the same line as SEQ ID NO. 29286 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 73038, or a homolog or fragment thereof, or a homolog thereof being depicted in Table I, application no. 5, column 8, in the same line as SEQ ID NO. 73038, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 73038 is depicted, plastidic is mentioned, or a mitochondrial transit peptide in case in Table I, application no. 5, in column 6 in the same line as SEQ ID NO. 73038 is depicted, mitochondrial is mentioned; which is operable linked, too.

In another embodiment thereof the expression cassette does not comprise an additional transit peptide in case in Table I, application no. 5, in column 6 in the same line as SEQ ID NO. 73038 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 74163, or a homolog or fragment thereof, or a homolog thereof being depicted in Table I, application no. 5, column 8, in the same line as SEQ ID NO. 74163, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 74163 is depicted, plastidic is mentioned, or a mitochondrial transit peptide in case in Table I, application no. 5, in column 6 in the same line as SEQ ID NO. 74163 is depicted, mitochondrial is mentioned; which is operable linked, too.

In another embodiment thereof the expression cassette does not comprise an additional transit peptide in case in Table I, application no. 5, in column 6 in the same line as SEQ ID NO. 74163 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6510, or a homolog or fragment thereof, or a homolog thereof being depicted in Table I, application no. 5, column 8, in the same line as SEQ ID NO. 6510, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 6510 is depicted, plastidic is mentioned, or a mitochondrial transit peptide in case in Table I, application no. 5, in column 6 in the same line as SEQ ID NO. 6510 is depicted, mitochondrial is mentioned; which is operable linked, too.

In another embodiment thereof the expression cassette does not comprise an additional transit peptide in case in Table I, application no. 5, in column 6 in the same line as SEQ ID NO. 6510 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 33596, or a homolog or fragment thereof, or a homolog thereof being depicted in Table I, application no. 5, column 8, in the same line as SEQ ID NO. 33596, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 33596 is depicted, plastidic is mentioned, or a mitochondrial transit peptide in case in Table I, application no. 5, in column 6 in the same line as SEQ ID NO. 33596 is depicted, mitochondrial is mentioned; which is operable linked, too.

In another embodiment thereof the expression cassette does not comprise an additional transit peptide in case in Table I, application no. 5, in column 6 in the same line as SEQ ID NO. 33596 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 74729, or a homolog or fragment thereof, or a homolog thereof being depicted in Table I, application no. 5, column 8, in the same line as SEQ ID NO. 74729, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 74729 is depicted, plastidic is mentioned, or a mitochondrial transit peptide in case in Table I, application no. 5, in column 6 in the same line as SEQ ID NO. 74729 is depicted, mitochondrial is mentioned; which is operable linked, too.

In another embodiment thereof the expression cassette does not comprise an additional transit peptide in case in Table I, application no. 5, in column 6 in the same line as SEQ ID NO. 74729 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 34889, or a homolog or fragment thereof, or a homolog thereof being depicted in Table I, application no. 5, column 8, in the same line as SEQ ID NO. 34889, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 34889 is depicted, plastidic is mentioned, or a mitochondrial transit peptide in case in Table I, application no. 5, in column 6 in the same line as SEQ ID NO. 34889 is depicted, mitochondrial is mentioned; which is operable linked, too.

In another embodiment thereof the expression cassette does not comprise an additional transit peptide in case in Table I, application no. 5, in column 6 in the same line as SEQ ID NO. 34889 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 98553, or a homolog or fragment thereof, or a homolog thereof being depicted in Table I, application no. 5, column 8, in the same line as SEQ ID NO. 98553, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 98553 is depicted, plastidic is mentioned, or a mitochondrial transit peptide in case in Table I, application no. 5, in column 6 in the same line as SEQ ID NO. 98553 is depicted, mitochondrial is mentioned; which is operable linked, too.

In another embodiment thereof the expression cassette does not comprise an additional transit peptide in case in Table I, application no. 5, in column 6 in the same line as SEQ ID NO. 98553 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 35204, or a homolog or fragment thereof, or a homolog thereof being depicted in Table I, application no. 5, column 8, in the same line as SEQ ID NO. 35204, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 35204 is depicted, plastidic is mentioned, or a mitochondrial transit peptide in case in Table I, application no. 5, in column 6 in the same line as SEQ ID NO. 35204 is depicted, mitochondrial is mentioned; which is operable linked, too.

In another embodiment thereof the expression cassette does not comprise an additional transit peptide in case in Table I, application no. 5, in column 6 in the same line as SEQ ID NO. 35204 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 35482, or a homolog or fragment thereof, or a homolog thereof being depicted in Table I, application no. 5, column 8, in the same line as SEQ ID NO. 35482, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 35482 is depicted, plastidic is mentioned, or a mitochondrial transit peptide in case in Table I, application no. 5, in column 6 in the same line as SEQ ID NO. 35482 is depicted, mitochondrial is mentioned; which is operable linked, too.

In another embodiment thereof the expression cassette does not comprise an additional transit peptide in case in Table I, application no. 5, in column 6 in the same line as SEQ ID NO. 35482 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7333, or a homolog or fragment thereof, or a homolog thereof being depicted in Table I, application no. 5, column 8, in the same line as SEQ ID NO. 7333, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 7333 is depicted, plastidic is mentioned, or a mitochondrial transit peptide in case in Table I, application no. 5, in column 6 in the same line as SEQ ID NO. 7333 is depicted, mitochondrial is mentioned; which is operable linked, too.

In another embodiment thereof the expression cassette does not comprise an additional transit peptide in case in Table I, application no. 5, in column 6 in the same line as SEQ ID NO. 7333 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 35967, or a homolog or fragment thereof, or a homolog thereof being depicted in Table I, application no. 5, column 8, in the same line as SEQ ID NO. 35967, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 35967 is depicted, plastidic is mentioned, or a mitochondrial transit peptide in case in Table I, application no. 5, in column 6 in the same line as SEQ ID NO. 35967 is depicted, mitochondrial is mentioned; which is operable linked, too.

In another embodiment thereof the expression cassette does not comprise an additional transit peptide in case in Table I, application no. 5, in column 6 in the same line as SEQ ID NO. 35967 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 36489, or a homolog or fragment thereof, or a homolog thereof being depicted in Table I, application no. 5, column 8, in the same line as SEQ ID NO. 36489, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 36489 is depicted, plastidic is mentioned, or a mitochondrial transit peptide in case in Table I, application no. 5, in column 6 in the same line as SEQ ID NO. 36489 is depicted, mitochondrial is mentioned; which is operable linked, too.

In another embodiment thereof the expression cassette does not comprise an additional transit peptide in case in Table I, application no. 5, in column 6 in the same line as SEQ ID NO. 36489 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 37394, or a homolog or fragment thereof, or a homolog thereof being depicted in Table I, application no. 5, column 8, in the same line as SEQ ID NO. 37394, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 37394 is depicted, plastidic is mentioned, or a mitochondrial transit peptide in case in Table I, application no. 5, in column 6 in the same line as SEQ ID NO. 37394 is depicted, mitochondrial is mentioned; which is operable linked, too.

In another embodiment thereof the expression cassette does not comprise an additional transit peptide in case in Table I, application no. 5, in column 6 in the same line as SEQ ID NO. 37394 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 98778, or a homolog or fragment thereof, or a homolog thereof being depicted in Table I, application no. 5, column 8, in the same line as SEQ ID NO. 98778, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 98778 is depicted, plastidic is mentioned, or a mitochondrial transit peptide in case in Table I, application no. 5, in column 6 in the same line as SEQ ID NO. 98778 is depicted, mitochondrial is mentioned; which is operable linked, too.

In another embodiment thereof the expression cassette does not comprise an additional transit peptide in case in Table I, application no. 5, in column 6 in the same line as SEQ ID NO. 98778 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 76157, or a homolog or fragment thereof, or a homolog thereof being depicted in Table I, application no. 5, column 8, in the same line as SEQ ID NO. 76157, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 76157 is depicted, plastidic is mentioned, or a mitochondrial transit peptide in case in Table I, application no. 5, in column 6 in the same line as SEQ ID NO. 76157 is depicted, mitochondrial is mentioned; which is operable linked, too.

In another embodiment thereof the expression cassette does not comprise an additional transit peptide in case in Table I, application no. 5, in column 6 in the same line as SEQ ID NO. 76157 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 37807, or a homolog or fragment thereof, or a homolog thereof being depicted in Table I, application no. 5, column 8, in the same line as SEQ ID NO. 37807, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 37807 is depicted, plastidic is mentioned, or a mitochondrial transit peptide in case in Table I, application no. 5, in column 6 in the same line as SEQ ID NO. 37807 is depicted, mitochondrial is mentioned; which is operable linked, too.

In another embodiment thereof the expression cassette does not comprise an additional transit peptide in case in Table I, application no. 5, in column 6 in the same line as SEQ ID NO. 37807 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 99188, or a homolog or fragment thereof, or a homolog thereof being depicted in Table I, application no. 5, column 8, in the same line as SEQ ID NO. 99188, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 99188 is depicted, plastidic is mentioned, or a mitochondrial transit peptide in case in Table I, application no. 5, in column 6 in the same line as SEQ ID NO. 99188 is depicted, mitochondrial is mentioned; which is operable linked, too.

In another embodiment thereof the expression cassette does not comprise an additional transit peptide in case in Table I, application no. 5, in column 6 in the same line as SEQ ID NO. 99188 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7947, or a homolog or fragment thereof, or a homolog thereof being depicted in Table I, application no. 5, column 8, in the same line as SEQ ID NO. 7947, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 7947 is depicted, plastidic is mentioned, or a mitochondrial transit peptide in case in Table I, application no. 5, in column 6 in the same line as SEQ ID NO. 7947 is depicted, mitochondrial is mentioned; which is operable linked, too.

In another embodiment thereof the expression cassette does not comprise an additional transit peptide in case in Table I, application no. 5, in column 6 in the same line as SEQ ID NO. 7947 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 99196, or a homolog or fragment thereof, or a homolog thereof being depicted in Table I, application no. 5, column 8, in the same line as SEQ ID NO. 99196, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 99196 is depicted, plastidic is mentioned, or a mitochondrial transit peptide in case in Table I, application no. 5, in column 6 in the same line as SEQ ID NO. 99196 is depicted, mitochondrial is mentioned; which is operable linked, too.

In another embodiment thereof the expression cassette does not comprise an additional transit peptide in case in Table I, application no. 5, in column 6 in the same line as SEQ ID NO. 99196 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 99210, or a homolog or fragment thereof, or a homolog thereof being depicted in Table I, application no. 5, column 8, in the same line as SEQ ID NO. 99210, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 99210 is depicted, plastidic is mentioned, or a mitochondrial transit peptide in case in Table I, application no. 5, in column 6 in the same line as SEQ ID NO. 99210 is depicted, mitochondrial is mentioned; which is operable linked, too.

In another embodiment thereof the expression cassette does not comprise an additional transit peptide in case in Table I, application no. 5, in column 6 in the same line as SEQ ID NO. 99210 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 79056, or a homolog or fragment thereof, or a homolog thereof being depicted in Table I, application no. 5, column 8, in the same line as SEQ ID NO. 79056, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 79056 is depicted, plastidic is mentioned, or a mitochondrial transit peptide in case in Table I, application no. 5, in column 6 in the same line as SEQ ID NO. 79056 is depicted, mitochondrial is mentioned; which is operable linked, too.

In another embodiment thereof the expression cassette does not comprise an additional transit peptide in case in Table I, application no. 5, in column 6 in the same line as SEQ ID NO. 79056 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 99371, or a homolog or fragment thereof, or a homolog thereof being depicted in Table I, application no. 5, column 8, in the same line as SEQ ID NO. 99371, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 99371 is depicted, plastidic is mentioned, or a mitochondrial transit peptide in case in Table I, application no. 5, in column 6 in the same line as SEQ ID NO. 99371 is depicted, mitochondrial is mentioned; which is operable linked, too.

In another embodiment thereof the expression cassette does not comprise an additional transit peptide in case in Table I, application no. 5, in column 6 in the same line as SEQ ID NO. 99371 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38947, or a homolog or fragment thereof, or a homolog thereof being depicted in Table I, application no. 5, column 8, in the same line as SEQ ID NO. 38947, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 38947 is depicted, plastidic is mentioned, or a mitochondrial transit peptide in case in Table I, application no. 5, in column 6 in the same line as SEQ ID NO. 38947 is depicted, mitochondrial is mentioned; which is operable linked, too.

In another embodiment thereof the expression cassette does not comprise an additional transit peptide in case in Table I, application no. 5, in column 6 in the same line as SEQ ID NO. 38947 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 99620, or a homolog or fragment thereof, or a homolog thereof being depicted in Table I, application no. 5, column 8, in the same line as SEQ ID NO. 99620, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 99620 is depicted, plastidic is mentioned, or a mitochondrial transit peptide in case in Table I, application no. 5, in column 6 in the same line as SEQ ID NO. 99620 is depicted, mitochondrial is mentioned; which is operable linked, too.

In another embodiment thereof the expression cassette does not comprise an additional transit peptide in case in Table I, application no. 5, in column 6 in the same line as SEQ ID NO. 99620 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 99898, or a homolog or fragment thereof, or a homolog thereof being depicted in Table I, application no. 5, column 8, in the same line as SEQ ID NO. 99898, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 99898 is depicted, plastidic is mentioned, or a mitochondrial transit peptide in case in Table I, application no. 5, in column 6 in the same line as SEQ ID NO. 99898 is depicted, mitochondrial is mentioned; which is operable linked, too.

In another embodiment thereof the expression cassette does not comprise an additional transit peptide in case in Table I, application no. 5, in column 6 in the same line as SEQ ID NO. 99898 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 100105, or a homolog or fragment thereof, or a homolog thereof being depicted in Table I, application no. 5, column 8, in the same line as SEQ ID NO. 100105, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 100105 is depicted, plastidic is mentioned, or a mitochondrial transit peptide in case in Table I, application no. 5, in column 6 in the same line as SEQ ID NO. 100105 is depicted, mitochondrial is mentioned; which is operable linked, too.

In another embodiment thereof the expression cassette does not comprise an additional transit peptide in case in Table I, application no. 5, in column 6 in the same line as SEQ ID NO. 100105 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 80906, or a homolog or fragment thereof, or a homolog thereof being depicted in Table I, application no. 5, column 8, in the same line as SEQ ID NO. 80906, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 80906 is depicted, plastidic is mentioned, or a mitochondrial transit peptide in case in Table I, application no. 5, in column 6 in the same line as SEQ ID NO. 80906 is depicted, mitochondrial is mentioned; which is operable linked, too.

In another embodiment thereof the expression cassette does not comprise an additional transit peptide in case in Table I, application no. 5, in column 6 in the same line as SEQ ID NO. 80906 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 81980, or a homolog or fragment thereof, or a homolog thereof being depicted in Table I, application no. 5, column 8, in the same line as SEQ ID NO. 81980, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 81980 is depicted, plastidic is mentioned, or a mitochondrial transit peptide in case in Table I, application no. 5, in column 6 in the same line as SEQ ID NO. 81980 is depicted, mitochondrial is mentioned; which is operable linked, too.

In another embodiment thereof the expression cassette does not comprise an additional transit peptide in case in Table I, application no. 5, in column 6 in the same line as SEQ ID NO. 81980 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 100543, or a homolog or fragment thereof, or a homolog thereof being depicted in Table I, application no. 5, column 8, in the same line as SEQ ID NO. 100543, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 100543 is depicted, plastidic is mentioned, or a mitochondrial transit peptide in case in Table I, application no. 5, in column 6 in the same line as SEQ ID NO. 100543 is depicted, mitochondrial is mentioned; which is operable linked, too.

In another embodiment thereof the expression cassette does not comprise an additional transit peptide in case in Table I, application no. 5, in column 6 in the same line as SEQ ID NO. 100543 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 42502, or a homolog or fragment thereof, or a homolog thereof being depicted in Table I, application no. 5, column 8, in the same line as SEQ ID NO. 42502, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 42502 is depicted, plastidic is mentioned, or a mitochondrial transit peptide in case in Table I, application no. 5, in column 6 in the same line as SEQ ID NO. 42502 is depicted, mitochondrial is mentioned; which is operable linked, too.

In another embodiment thereof the expression cassette does not comprise an additional transit peptide in case in Table I, application no. 5, in column 6 in the same line as SEQ ID NO. 42502 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 100675, or a homolog or fragment thereof, or a homolog thereof being depicted in Table I, application no. 5, column 8, in the same line as SEQ ID NO. 100675, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 100675 is depicted, plastidic is mentioned, or a mitochondrial transit peptide in case in Table I, application no. 5, in column 6 in the same line as SEQ ID NO. 100675 is depicted, mitochondrial is mentioned; which is operable linked, too.

In another embodiment thereof the expression cassette does not comprise an additional transit peptide in case in Table I, application no. 5, in column 6 in the same line as SEQ ID NO. 100675 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 101327, or a homolog or fragment thereof, or a homolog thereof being depicted in Table I, application no. 5, column 8, in the same line as SEQ ID NO. 101327, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 101327 is depicted, plastidic is mentioned, or a mitochondrial transit peptide in case in Table I, application no. 5, in column 6 in the same line as SEQ ID NO. 101327 is depicted, mitochondrial is mentioned; which is operable linked, too.

In another embodiment thereof the expression cassette does not comprise an additional transit peptide in case in Table I, application no. 5, in column 6 in the same line as SEQ ID NO. 101327 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 44372, or a homolog or fragment thereof, or a homolog thereof being depicted in Table I, application no. 5, column 8, in the same line as SEQ ID NO. 44372, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 44372 is depicted, plastidic is mentioned, or a mitochondrial transit peptide in case in Table I, application no. 5, in column 6 in the same line as SEQ ID NO. 44372 is depicted, mitochondrial is mentioned; which is operable linked, too.

In another embodiment thereof the expression cassette does not comprise an additional transit peptide in case in Table I, application no. 5, in column 6 in the same line as SEQ ID NO. 44372 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 10740, or a homolog or fragment thereof, or a homolog thereof being depicted in Table I, application no. 5, column 8, in the same line as SEQ ID NO. 10740, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 10740 is depicted, plastidic is mentioned, or a mitochondrial transit peptide in case in Table I, application no. 5, in column 6 in the same line as SEQ ID NO. 10740 is depicted, mitochondrial is mentioned; which is operable linked, too.

In another embodiment thereof the expression cassette does not comprise an additional transit peptide in case in Table I, application no. 5, in column 6 in the same line as SEQ ID NO. 10740 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 44609, or a homolog or fragment thereof, or a homolog thereof being depicted in Table I, application no. 5, column 8, in the same line as SEQ ID NO. 44609, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 44609 is depicted, plastidic is mentioned, or a mitochondrial transit peptide in case in Table I, application no. 5, in column 6 in the same line as SEQ ID NO. 44609 is depicted, mitochondrial is mentioned; which is operable linked, too.

In another embodiment thereof the expression cassette does not comprise an additional transit peptide in case in Table I, application no. 5, in column 6 in the same line as SEQ ID NO. 44609 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 45321, or a homolog or fragment thereof, or a homolog thereof being depicted in Table I, application no. 5, column 8, in the same line as SEQ ID NO. 45321, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 45321 is depicted, plastidic is mentioned, or a mitochondrial transit peptide in case in Table I, application no. 5, in column 6 in the same line as SEQ ID NO. 45321 is depicted, mitochondrial is mentioned; which is operable linked, too.

In another embodiment thereof the expression cassette does not comprise an additional transit peptide in case in Table I, application no. 5, in column 6 in the same line as SEQ ID NO. 45321 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 46515, or a homolog or fragment thereof, or a homolog thereof being depicted in Table I, application no. 5, column 8, in the same line as SEQ ID NO. 46515, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 46515 is depicted, plastidic is mentioned, or a mitochondrial transit peptide in case in Table I, application no. 5, in column 6 in the same line as SEQ ID NO. 46515 is depicted, mitochondrial is mentioned; which is operable linked, too.

In another embodiment thereof the expression cassette does not comprise an additional transit peptide in case in Table I, application no. 5, in column 6 in the same line as SEQ ID NO. 46515 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 84198, or a homolog or fragment thereof, or a homolog thereof being depicted in Table I, application no. 5, column 8, in the same line as SEQ ID NO. 84198, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 84198 is depicted, plastidic is mentioned, or a mitochondrial transit peptide in case in Table I, application no. 5, in column 6 in the same line as SEQ ID NO. 84198 is depicted, mitochondrial is mentioned; which is operable linked, too.

In another embodiment thereof the expression cassette does not comprise an additional transit peptide in case in Table I, application no. 5, in column 6 in the same line as SEQ ID NO. 84198 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 101780, or a homolog or fragment thereof, or a homolog thereof being depicted in Table I, application no. 5, column 8, in the same line as SEQ ID NO. 101780, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 101780 is depicted, plastidic is mentioned, or a mitochondrial transit peptide in case in Table I, application no. 5, in column 6 in the same line as SEQ ID NO. 101780 is depicted, mitochondrial is mentioned; which is operable linked, too.

In another embodiment thereof the expression cassette does not comprise an additional transit peptide in case in Table I, application no. 5, in column 6 in the same line as SEQ ID NO. 101780 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 49143, or a homolog or fragment thereof, or a homolog thereof being depicted in Table I, application no. 5, column 8, in the same line as SEQ ID NO. 49143, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 49143 is depicted, plastidic is mentioned, or a mitochondrial transit peptide in case in Table I, application no. 5, in column 6 in the same line as SEQ ID NO. 49143 is depicted, mitochondrial is mentioned; which is operable linked, too.

In another embodiment thereof the expression cassette does not comprise an additional transit peptide in case in Table I, application no. 5, in column 6 in the same line as SEQ ID NO. 49143 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 50339, or a homolog or fragment thereof, or a homolog thereof being depicted in Table I, application no. 5, column 8, in the same line as SEQ ID NO. 50339, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 50339 is depicted, plastidic is mentioned, or a mitochondrial transit peptide in case in Table I, application no. 5, in column 6 in the same line as SEQ ID NO. 50339 is depicted, mitochondrial is mentioned; which is operable linked, too.

In another embodiment thereof the expression cassette does not comprise an additional transit peptide in case in Table I, application no. 5, in column 6 in the same line as SEQ ID NO. 50339 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 101707, or a homolog or fragment thereof, or a homolog thereof being depicted in Table I, application no. 5, column 8, in the same line as SEQ ID NO. 101707, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 101707 is depicted, plastidic is mentioned, or a mitochondrial transit peptide in case in Table I, application no. 5, in column 6 in the same line as SEQ ID NO. 101707 is depicted, mitochondrial is mentioned; which is operable linked, too.

In another embodiment thereof the expression cassette does not comprise an additional transit peptide in case in Table I, application no. 5, in column 6 in the same line as SEQ ID NO. 101707 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 52634, or a homolog or fragment thereof, or a homolog thereof being depicted in Table I, application no. 5, column 8, in the same line as SEQ ID NO. 52634, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 52634 is depicted, plastidic is mentioned, or a mitochondrial transit peptide in case in Table I, application no. 5, in column 6 in the same line as SEQ ID NO. 52634 is depicted, mitochondrial is mentioned; which is operable linked, too.

In another embodiment thereof the expression cassette does not comprise an additional transit peptide in case in Table I, application no. 5, in column 6 in the same line as SEQ ID NO. 52634 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 54337, or a homolog or fragment thereof, or a homolog thereof being depicted in Table I, application no. 5, column 8, in the same line as SEQ ID NO. 54337, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 54337 is depicted, plastidic is mentioned, or a mitochondrial transit peptide in case in Table I, application no. 5, in column 6 in the same line as SEQ ID NO. 54337 is depicted, mitochondrial is mentioned; which is operable linked, too.

In another embodiment thereof the expression cassette does not comprise an additional transit peptide in case in Table I, application no. 5, in column 6 in the same line as SEQ ID NO. 54337 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 11471, or a homolog or fragment thereof, or a homolog thereof being depicted in Table I, application no. 5, column 8, in the same line as SEQ ID NO. 11471, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 11471 is depicted, plastidic is mentioned, or a mitochondrial transit peptide in case in Table I, application no. 5, in column 6 in the same line as SEQ ID NO. 11471 is depicted, mitochondrial is mentioned; which is operable linked, too.

In another embodiment thereof the expression cassette does not comprise an additional transit peptide in case in Table I, application no. 5, in column 6 in the same line as SEQ ID NO. 11471 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 86540, or a homolog or fragment thereof, or a homolog thereof being depicted in Table I, application no. 5, column 8, in the same line as SEQ ID NO. 86540, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 86540 is depicted, plastidic is mentioned, or a mitochondrial transit peptide in case in Table I, application no. 5, in column 6 in the same line as SEQ ID NO. 86540 is depicted, mitochondrial is mentioned; which is operable linked, too.

In another embodiment thereof the expression cassette does not comprise an additional transit peptide in case in Table I, application no. 5, in column 6 in the same line as SEQ ID NO. 86540 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 87397, or a homolog or fragment thereof, or a homolog thereof being depicted in Table I, application no. 5, column 8, in the same line as SEQ ID NO. 87397, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 87397 is depicted, plastidic is mentioned, or a mitochondrial transit peptide in case in Table I, application no. 5, in column 6 in the same line as SEQ ID NO. 87397 is depicted, mitochondrial is mentioned; which is operable linked, too.

In another embodiment thereof the expression cassette does not comprise an additional transit peptide in case in Table I, application no. 5, in column 6 in the same line as SEQ ID NO. 87397 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 57235, or a homolog or fragment thereof, or a homolog thereof being depicted in Table I, application no. 5, column 8, in the same line as SEQ ID NO. 57235, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 57235 is depicted, plastidic is mentioned, or a mitochondrial transit peptide in case in Table I, application no. 5, in column 6 in the same line as SEQ ID NO. 57235 is depicted, mitochondrial is mentioned; which is operable linked, too.

In another embodiment thereof the expression cassette does not comprise an additional transit peptide in case in Table I, application no. 5, in column 6 in the same line as SEQ ID NO. 57235 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 101776, or a homolog or fragment thereof, or a homolog thereof being depicted in Table I, application no. 5, column 8, in the same line as SEQ ID NO. 101776, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 101776 is depicted, plastidic is mentioned, or a mitochondrial transit peptide in case in Table I, application no. 5, in column 6 in the same line as SEQ ID NO. 101776 is depicted, mitochondrial is mentioned; which is operable linked, too.

In another embodiment thereof the expression cassette does not comprise an additional transit peptide in case in Table I, application no. 5, in column 6 in the same line as SEQ ID NO. 101776 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 58731, or a homolog or fragment thereof, or a homolog thereof being depicted in Table I, application no. 5, column 8, in the same line as SEQ ID NO. 58731, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 58731 is depicted, plastidic is mentioned, or a mitochondrial transit peptide in case in Table I, application no. 5, in column 6 in the same line as SEQ ID NO. 58731 is depicted, mitochondrial is mentioned; which is operable linked, too.

In another embodiment thereof the expression cassette does not comprise an additional transit peptide in case in Table I, application no. 5, in column 6 in the same line as SEQ ID NO. 58731 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 59370, or a homolog or fragment thereof, or a homolog thereof being depicted in Table I, application no. 5, column 8, in the same line as SEQ ID NO. 59370, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 59370 is depicted, plastidic is mentioned, or a mitochondrial transit peptide in case in Table I, application no. 5, in column 6 in the same line as SEQ ID NO. 59370 is depicted, mitochondrial is mentioned; which is operable linked, too.

In another embodiment thereof the expression cassette does not comprise an additional transit peptide in case in Table I, application no. 5, in column 6 in the same line as SEQ ID NO. 59370 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12698, or a homolog or fragment thereof, or a homolog thereof being depicted in Table I, application no. 5, column 8, in the same line as SEQ ID NO. 12698, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 12698 is depicted, plastidic is mentioned, or a mitochondrial transit peptide in case in Table I, application no. 5, in column 6 in the same line as SEQ ID NO. 12698 is depicted, mitochondrial is mentioned; which is operable linked, too.

In another embodiment thereof the expression cassette does not comprise an additional transit peptide in case in Table I, application no. 5, in column 6 in the same line as SEQ ID NO. 12698 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 88879, or a homolog or fragment thereof, or a homolog thereof being depicted in Table I, application no. 5, column 8, in the same line as SEQ ID NO. 88879, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 88879 is depicted, plastidic is mentioned, or a mitochondrial transit peptide in case in Table I, application no. 5, in column 6 in the same line as SEQ ID NO. 88879 is depicted, mitochondrial is mentioned; which is operable linked, too.

In another embodiment thereof the expression cassette does not comprise an additional transit peptide in case in Table I, application no. 5, in column 6 in the same line as SEQ ID NO. 88879 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 63544, or a homolog or fragment thereof, or a homolog thereof being depicted in Table I, application no. 5, column 8, in the same line as SEQ ID NO. 63544, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 63544 is depicted, plastidic is mentioned, or a mitochondrial transit peptide in case in Table I, application no. 5, in column 6 in the same line as SEQ ID NO. 63544 is depicted, mitochondrial is mentioned; which is operable linked, too.

In another embodiment thereof the expression cassette does not comprise an additional transit peptide in case in Table I, application no. 5, in column 6 in the same line as SEQ ID NO. 63544 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 64148, or a homolog or fragment thereof, or a homolog thereof being depicted in Table I, application no. 5, column 8, in the same line as SEQ ID NO. 64148, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 64148 is depicted, plastidic is mentioned, or a mitochondrial transit peptide in case in Table I, application no. 5, in column 6 in the same line as SEQ ID NO. 64148 is depicted, mitochondrial is mentioned; which is operable linked, too.

In another embodiment thereof the expression cassette does not comprise an additional transit peptide in case in Table I, application no. 5, in column 6 in the same line as SEQ ID NO. 64148 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 101855, or a homolog or fragment thereof, or a homolog thereof being depicted in Table I, application no. 5, column 8, in the same line as SEQ ID NO. 101855, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 101855 is depicted, plastidic is mentioned, or a mitochondrial transit peptide in case in Table I, application no. 5, in column 6 in the same line as SEQ ID NO. 101855 is depicted, mitochondrial is mentioned; which is operable linked, too.

In another embodiment thereof the expression cassette does not comprise an additional transit peptide in case in Table I, application no. 5, in column 6 in the same line as SEQ ID NO. 101855 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 102015, or a homolog or fragment thereof, or a homolog thereof being depicted in Table I, application no. 5, column 8, in the same line as SEQ ID NO. 102015, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 102015 is depicted, plastidic is mentioned, or a mitochondrial transit peptide in case in Table I, application no. 5, in column 6 in the same line as SEQ ID NO. 102015 is depicted, mitochondrial is mentioned; which is operable linked, too.

In another embodiment thereof the expression cassette does not comprise an additional transit peptide in case in Table I, application no. 5, in column 6 in the same line as SEQ ID NO. 102015 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 14843, or a homolog or fragment thereof, or a homolog thereof being depicted in Table I, application no. 5, column 8, in the same line as SEQ ID NO. 14843, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 14843 is depicted, plastidic is mentioned, or a mitochondrial transit peptide in case in Table I, application no. 5, in column 6 in the same line as SEQ ID NO. 14843 is depicted, mitochondrial is mentioned; which is operable linked, too.

In another embodiment thereof the expression cassette does not comprise an additional transit peptide in case in Table I, application no. 5, in column 6 in the same line as SEQ ID NO. 14843 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 66274, or a homolog or fragment thereof, or a homolog thereof being depicted in Table I, application no. 5, column 8, in the same line as SEQ ID NO. 66274, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 66274 is depicted, plastidic is mentioned, or a mitochondrial transit peptide in case in Table I, application no. 5, in column 6 in the same line as SEQ ID NO. 66274 is depicted, mitochondrial is mentioned; which is operable linked, too.

In another embodiment thereof the expression cassette does not comprise an additional transit peptide in case in Table I, application no. 5, in column 6 in the same line as SEQ ID NO. 66274 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 102061, or a homolog or fragment thereof, or a homolog thereof being depicted in Table I, application no. 5, column 8, in the same line as SEQ ID NO. 102061, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 102061 is depicted, plastidic is mentioned, or a mitochondrial transit peptide in case in Table I, application no. 5, in column 6 in the same line as SEQ ID NO. 102061 is depicted, mitochondrial is mentioned; which is operable linked, too.

In another embodiment thereof the expression cassette does not comprise an additional transit peptide in case in Table I, application no. 5, in column 6 in the same line as SEQ ID NO. 102061 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 92339, or a homolog or fragment thereof, or a homolog thereof being depicted in Table I, application no. 5, column 8, in the same line as SEQ ID NO. 92339, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 92339 is depicted, plastidic is mentioned, or a mitochondrial transit peptide in case in Table I, application no. 5, in column 6 in the same line as SEQ ID NO. 92339 is depicted, mitochondrial is mentioned; which is operable linked, too.

In another embodiment thereof the expression cassette does not comprise an additional transit peptide in case in Table I, application no. 5, in column 6 in the same line as SEQ ID NO. 92339 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 92540, or a homolog or fragment thereof, or a homolog thereof being depicted in Table I, application no. 5, column 8, in the same line as SEQ ID NO. 92540, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 92540 is depicted, plastidic is mentioned, or a mitochondrial transit peptide in case in Table I, application no. 5, in column 6 in the same line as SEQ ID NO. 92540 is depicted, mitochondrial is mentioned; which is operable linked, too.

In another embodiment thereof the expression cassette does not comprise an additional transit peptide in case in Table I, application no. 5, in column 6 in the same line as SEQ ID NO. 92540 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 67998, or a homolog or fragment thereof, or a homolog thereof being depicted in Table I, application no. 5, column 8, in the same line as SEQ ID NO. 67998, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 67998 is depicted, plastidic is mentioned, or a mitochondrial transit peptide in case in Table I, application no. 5, in column 6 in the same line as SEQ ID NO. 67998 is depicted, mitochondrial is mentioned; which is operable linked, too.

In another embodiment thereof the expression cassette does not comprise an additional transit peptide in case in Table I, application no. 5, in column 6 in the same line as SEQ ID NO. 67998 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 98445, or a homolog or fragment thereof, or a homolog thereof being depicted in Table I, application no. 5, column 8, in the same line as SEQ ID NO. 98445, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 98445 is depicted, plastidic is mentioned, or a mitochondrial transit peptide in case in Table I, application no. 5, in column 6 in the same line as SEQ ID NO. 98445 is depicted, mitochondrial is mentioned; which is operable linked, too.

In another embodiment thereof the expression cassette does not comprise an additional transit peptide in case in Table I, application no. 5, in column 6 in the same line as SEQ ID NO. 98445 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68413, or a homolog or fragment thereof, or a homolog thereof being depicted in Table I, application no. 5, column 8, in the same line as SEQ ID NO. 68413, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 5, in column 6 in the same line as SEQ ID NO. 68413 is depicted, plastidic is mentioned, or a mitochondrial transit peptide in case in Table I, application no. 5, in column 6 in the same line as SEQ ID NO. 68413 is depicted, mitochondrial is mentioned; which is operable linked, too.

In another embodiment thereof the expression cassette does not comprise an additional transit peptide in case in Table I, application no. 5, in column 6 in the same line as SEQ ID NO. 68413 is depicted, non-targeted is mentioned.

[0165.1.1.5] to [0170.1.1.5] for the disclosure of these paragraphs see [0165.1.1.1] to [0170.1.1.1] above.

The invention further provides a vector, comprising a nucleic acid molecule comprising 1) a nucleic acid molecule selected from the group consisting of:
(a) a nucleic acid molecule encoding a polypeptide shown in Table II, column 5 or 8, application no. 5, preferably shown in Table II A, application no. 5, in column 5, or in Table II A, application no. 5, column 8, or in Table II B, application no. 5, column 8, or a homolog or a fragment thereof;
(b) a nucleic acid molecule shown in Table I, application no. 5, column 5 or 8, preferably shown in Table I A, application no. 5, in column 5, or in Table I A, application no. 5, column 8, or in Table I B, application no. 5, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 5, preferably shown in Table II A, application no. 5, in column 5, or in Table II A, application no. 5, column 8, or in Table II B, application no. 5, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 5, preferably shown in Table I A, application no. 5, in column 5, or in Table I A, application no. 5, column 8, or in Table I B, application no. 5, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 5;
(i) a nucleic acid molecule encoding a polypeptide having the activity represented by a protein as depicted in column 5 of Table II, application no. 5;
(j) a nucleic acid molecule comprising a polynucleotide which is obtained by amplifying a cDNA library or a genomic library using the primers in Table III, application no. 5, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 5.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 5, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 5.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 5, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 5, in the same line as of the respective nucleic acid molecule according to 1) “plastidic” is depicted, or encoding a mitochondrial transit peptide, especially in case in column 6 of Table I, application no. 5, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 5, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 5, in the same line as of the respective nucleic acid molecule according to 1) “plastidic” is depicted, or encoding a mitochondrial transit peptide, especially in case in column 6 of Table I, application no. 5, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operable linked; a nucleic acid molecule encoding a transit peptide, especially in case in column 6 of Table I, application no. 5, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 5, in the same line as of the respective nucleic acid molecule according to 1) “plastidic” is depicted, or encoding a mitochondrial transit peptide, especially in case in column 6 of Table I, application no. 5, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 5 (in the same line).

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 5, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 5.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 5, in the same line as of the respective nucleic acid molecule according to 1) “plastidic” is depicted, or encoding a mitochondrial transit peptide, especially in case in column 6 of Table I, application no. 5, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 5, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 5, in the same line as of the respective nucleic acid molecule according to 1) “plastidic” is depicted, or encoding a mitochondrial transit peptide, especially in case in column 6 of Table I, application no. 5, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 5 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 5, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 5 (in the same line) and confers a generation or an increase of the respective fine chemical in a non-human organism or a part thereof as compared to a corresponding wild-type non-human organism or part thereof. In a preferred embodiment of said vector said nucleic acid molecule according to 1 (a), (b), (c), (d), (e), (f), (g), (h), (i), (j) or (k), said promoter and optionally said nucleic acid molecule encoding a transit peptide are encompassed in an expression cassette. Accordingly in an embodiment the invention provides a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

The invention further provides a vector comprising a nucleic acid molecule comprising 1) a nucleic acid molecule selected from the group consisting of:
(a) a nucleic acid molecule encoding a polypeptide shown in Table II, column 5 or 8, application no. 5, preferably shown in Table II A, application no. 5, in column 5, or in Table II A, application no. 5, column 8, or in Table II B, application no. 5, column 8, or a homolog or a fragment thereof;
(b) a nucleic acid molecule shown in Table I, application no. 5, column 5 or 8, preferably shown in Table I A, application no. 5, in column 5, or in Table I A, application no. 5, column 8, or in Table I B, application no. 5, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 5, preferably shown in Table II A, application no. 5, in column 5, or in Table II A, application no. 5, column 8, or in Table II B, application no. 5, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 5, preferably shown in Table I A, application no. 5, in column 5, or in Table I A, application no. 5, column 8, or in Table I B, application no. 5, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 5;
(i) a nucleic acid molecule encoding a polypeptide having the activity represented by a protein as depicted in column 5 of Table II, application no. 5;
(j) a nucleic acid molecule comprising a polynucleotide which is obtained by amplifying a cDNA library or a genomic library using the primers in Table III, application no. 5, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto;
   whereby, preferably, the nucleic acid molecule according to 1 (a), (b), (c), (d), (e), (f), (g), (h), (i), (j) or (k) distinguishes over the sequence depicted in Table I A and/or I B, application no. 5, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 5, column 5 or 8, or the coding region thereof, respectively. In another embodiment, the nucleic acid molecule of the present invention, preferably the coding region thereof, is 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 5, column 5 or 8, or the coding region thereof, respectively. In a further embodiment the nucleic acid molecule does not encode the polypeptide sequence shown in Table II A and/or II B, application no. 5, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 5, column 5 or 8 and does not encode a protein of the sequence shown in Table II A and/or II B, application no. 5, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 5, column 5 and 8, or the coding region thereof. In a further embodiment, the protein of the present invention is at least 30%,40%, 50%, 60%, 70%, 80%, 90%, 95% identical to protein sequence depicted in Table II A and/or II B, application no. 5, column 5 or 8 but less than 100%, preferably less than 99.999%, 99.99% or 99.9%, more preferably less than 99%, 985, 97%, 96% or 95% identical to the sequence shown in Table II A and/or II B, application no. 5, column 5 or 8.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 5.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 5, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 5.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 5, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 5, in the same line as of the respective nucleic acid molecule according to 1) “plastidic” is depicted, or encoding a mitochondrial transit peptide, especially in case in column 6 of Table I, application no. 5, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 5, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 5, in the same line as of the respective nucleic acid molecule according to 1) “plastidic” is depicted, or encoding a mitochondrial transit peptide, especially in case in column 6 of Table I, application no. 5, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 5, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 5, in the same line as of the respective nucleic acid molecule according to 1) “plastidic” is depicted, or encoding a mitochondrial transit peptide, especially in case in column 6 of Table I, application no. 5, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 5.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 5, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 5.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 5, in the same line as of the respective nucleic acid molecule according to 1) “plastidic” is depicted, or encoding a mitochondrial transit peptide, especially in case in column 6 of Table I, application no. 5, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 5, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 5, in the same line as of the respective nucleic acid molecule according to 1) “plastidic” is depicted, or encoding a mitochondrial transit peptide, especially in case in column 6 of Table I, application no. 5, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 5 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 5, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 5 and confers a generation or an increase of the respective fine chemical in a non-human organism or a part thereof as compared to a corresponding wild-type non-human organism or part thereof.

[0181.1.1.5] to [0209.1.1.5] for the disclosure of these paragraphs see [0181.1.1.1] to [0209.1.1.1] above.

The genes of the invention, coding for an activity selected from the group consisting of 3-deoxy-7-phosphoheptulonate synthase, 3-ketoacyl-CoA thiolase, acetyltransferase, aldehyde dehydrogenase, amino acid transporter, aromatic amino acid aminotransferase, At1g19800-protein, At2g33510-protein, At4g32480-protein, At5g16650-protein, at5g21910-protein, ATP synthase subunit E, ATP-binding component of a transport system, auxin response factor, b1163-protein, b1346-protein, b1522-protein, b1796-protein, b2739-protein, b2849-protein, b3989-protein, b4029-protein, b4121-protein, binding-protein-dependent transport systems inner membrane component, branched-chain amino acid permease, calcium-dependent protein kinase, carbon dioxide concentrating mechanism protein, cardiolipin synthase, CBL-interacting protein kinase, Chaperone protein CIpB, choline kinase, coproporphyrinogen III oxidase, cullin, cyclin, cyclin D , cystathionine gamma-synthase, DNA mismatch repair protein, electron transfer flavoprotein subunit beta, elongation factor Tu, enoyl-CoA hydratase, eukaryotic translation initiation factor, eukaryotic translation initiation factor 5, fimbrial-like protein, flavodoxin, geranylgeranyl pyrophosphate synthase, gibberellin 20-oxidase, glucose dehydrogenase, glucose-6-phosphate 1-dehydrogenase, glutamate-ammonia-ligase, glutamine synthetase, glutaredoxin, glycerol kinase, glycosyl transferase, harpin-induced family protein, histone acetyltransferase, hydrolase, imidazole glycerol phosphate synthase subunit, isochorismate synthase, isopentenyl diphosphate isomerase, Leucyl/phenylalanyl-tRNA-protein transferase, lipase, L-ribulose-5-phosphate 4-epimerase, malate dehydrogenase, malic enzyme, methyl-accepting chemotaxis protein, methyltransferase, monthiol glutaredoxin, NAD(P)H-quinone oxidoreductase subunit, nitrate/nitrite transport protein, photosystem II protein, polygalacturonase, precorrin methylase, protein kinase, protein phosphatase, putative transport system permease protein, pyruvate kinase, recombinase A, RNA binding protein, RNA-binding protein, Sec-independent protein translocase subunit, short-chain alcohol dehydrogenase family, slr1107-protein, threonine synthase, transaldolase, transcription factor, transcriptional regulator, transport protein, trehalose-phosphatase, tryptophan biosynthesis protein, yfl054c-protein, and Zm_{—}4842_BE510522-protein are also called “FCRP genes”.

[0211.1.1.5] to [0225.1.1.5] for the disclosure of these paragraphs see [0211.1.1.1] to [0225.1.1.1] above.

In addition to the sequence mentioned in Table I, application no. 5, column 5 or 8, preferably the coding region thereof, or its homologs or fragments, it may be advantageous additionally to express and/or mutate further genes in the non-human organisms. Especially advantageously, additionally at least one further gene of the biosynthetic pathway of the fine chemical is expressed in the non-human organisms such as plants or microorganisms. It is also possible that the regulation of the natural genes has been modified advantageously so that the gene and/or its gene product is no longer subject to the regulatory mechanisms which exist in the non-human organisms. This leads to an increased synthesis of the respective amino acid since, for example, feedback regulations no longer exist to the same extent or not at all. In addition it might be advantageously to combine the nucleic acids sequences of the invention containing the sequences shown in the respective line in Table I, application no. 5, column 5 or 8, preferably the coding region thereof, or homologs or fragments thereof, with genes which generally support or enhance the growth or yield of the target non-human organisms, for example genes which lead to faster growth rate of non human organism like microorganisms or plants or genes which produce stress-, pathogen-, or herbicide-resistant plants.

[0227.1.1.5] to [0239.1.1.5] for the disclosure of these paragraphs see [0227.1.1.1] to [0239.1.1.1] above.

In an embodiment of the present invention the nucleic acid construct of the present invention comprises a nucleic acid molecule as depicted in the respective line in Table I, application no. 5, column 5 or 8, preferably the coding region thereof, a fragment or a homolog thereof, and a plant promoter, such as the Big 35S promoter, the DC3 promoter, the PC Ubi promoter, the LegB4-promoter, the Super promoter, the USP promoter or the like. In another embodiment the nucleic acid construct of the present invention comprises a nucleic acid molecule as depicted in the respective line in Table I, application no. 5, column 5 or 8, preferably the coding region thereof, a fragment or a homolog thereof, and a plant promoter such as, for example, the USP, the LegB4-, the DC3 promoter or the ubiquitin promoter from parsley or other herein mentioned promoters. In a further embodiment the nucleic acid construct of the present invention comprises a nucleic acid molecule as depicted in the respective line in Table I, application no. 5, column 5 or 8, preferably the coding region thereof, a fragment or a homolog thereof, and a plant promoter, such as the Big 35S promoter, the PC Ubi promoter, the Super promoter, the USP promoter or the like. In addition different terminators may advantageously be used in these nucleic acid constructs; examples for transcriptional termination are polyadenylation signals.

[0241.1.1.5] to [0245.1.1.5] for the disclosure of these paragraphs see [0241.1.1.1] to 0245.1.1.1] above.

Other preferred sequences for use in operable linkage in gene expression constructs are targeting sequences, which are required for targeting the gene product into specific cell compartments (for a review, see Kermode, Crit. Rev. Plant Sci. 15 (4), 285 (1996) and references cited therein), for example into the vacuole, the nucleus, all types of plastids, such as amyloplasts, chloroplasts, chromoplasts, the extracellular space, the mitochondria, the endoplasmic reticulum, elaioplasts, peroxisomes, glycosomes, and other compartments of cells or extracellular; preferred are sequences, which are involved in targeting to plastids as mentioned above. Sequences, which must be mentioned in this context are, in particular, the signal-peptide- or transit-peptide-encoding sequences which are known per se. For example, plastid-transit-peptide-encoding sequences enable the targeting of the expression product into the plastids of a plant cell. Targeting sequences are also known for eukaryotic and to a lower extent for prokaryotic organisms and can advantageously be operable linked with the nucleic acid molecule of the present invention as shown in Table I, application no. 5, columns 5 and 8, preferably the coding region thereof, or homologs or fragments thereof, and described herein to achieve an expression in one of said compartments or extracellular.

[0247.1.1.5] to [0266.1.1.5] for the disclosure of these paragraphs see [0247.1.1.1] to [0266.1.1.1] above.

An embodiment of the invention relates to a vector where the nucleic acid molecule according to the invention is linked operably to regulatory sequences which permit the expression in a prokaryotic or eukaryotic host.

A further embodiment of the invention relates to a vector in which a nucleic acid sequence encoding one of the polypeptides shown in the respective line in Table II, application no. 5, columns 5 or 8, or homologs or fragments thereof, is functionally linked to a targeting sequence such as a plastidial or mitochondrial targeting sequence. A further embodiment of the invention relates to a vector in which a nucleic acid sequence encoding one of the polypeptides shown in the respective line in Table II, application no. 5, columns 5 or 8, or homologs or fragments thereof, is functionally linked to (a) regulatory sequence which permit the expression in an organelle, such as plastids or mitochondria, preferably plastids.

A further embodiment of the invention relates to a vector which comprises a nucleic acid sequence encoding one of the polypeptides shown in the respective line in Table II, application no. 5, columns 5 or 8, or homologs or fragments thereof, but no additional targeting sequence.

[0268.1.1.5] to [0273.1.1.5] for the disclosure of these paragraphs see [0268.1.1.1] to [0273.1.1.1] above.

A further object of the invention relates to the use of a nucleic acid construct, e.g. an expression cassette, comprising one or more nucleic acid molecules encoding one or more polypeptides as depicted in the respective line(s) in Table II, application no. 5, columns 5 or 8, or homologs or fragments thereof, or comprising one or more nucleic acid molecules as depicted in the respective line(s) in Table I, application no. 5, columns 5 or 8, preferably the coding region thereof, or homologs or fragments thereof, or comprising a nucleic acid molecule hybridizing therewith for the transformation of a microorganism or a plant cell, plant tissues or a part of a plant.

In doing so, depending on the choice of promoter, the nucleic acid molecules shown in the respective line in Table I, application no. 5, column 5 or 8, preferably the coding region thereof, or homologs or fragments thereof, or the nucleic acid molecules encoding a polypeptide as depicted in the respective line in Table II, application no. 5, column 5 or 8, or homologs or fragments thereof, can be expressed in the whole plant or specifically e.g. in the leaves, in the seeds, the nodules, in roots, in the stem or other parts of the plant. Those transgenic plants overproducing sequences, e.g. as depicted in the respective line in Table I, application no. 5, column 5 or 8, preferably the coding region thereof, or homologs or fragments thereof, the plant cell, plant tissues, plants, the reproductive material thereof, or parts thereof are a further object of the present invention.

Within the framework of the present invention, the production of the tryptophan is due to the generation or over-expression of one or more polypeptides as depicted in the respective line(s) in Table II, application no. 5, column 5 or 8, or homologs or fragments thereof, or encoded by the corresponding nucleic acid molecules as depicted in the respective line(s) in Table I, application no. 5, column 5 or 8, preferably the coding region thereof, or homologs or fragments thereof, in the non-human organism according to the invention, advantageously in the transgenic microorganism or plant according to the invention, at least for the duration of at least one plant generation.

A constitutive expression of the polypeptide as depicted in the respective line in Table II, application no. 5, column 5 or 8, or homologs or fragments thereof, or encoded by the respective nucleic acid molecule as depicted in the respective line in Table I, application no. 5, column 5 or 8, preferably the coding region thereof, or homologs or fragments thereof, may be advantageous. On the other hand, an inducible expression may also appear desirable. Expression of the polypeptide sequences of the invention can be either directed to the cytosol or to the organelles, such as plastids or mitochondria, preferably the plastids of the host cells, preferably the plant cells, or non-targeted.

for the disclosure of this paragraph see [0278.1.1.1] above

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 5, column 5 or 8, preferably shown in Table II A, application no. 5, column 5, or in Table II A, application no. 5, column 8, or in Table II B, application no. 5, column 8, or a homolog or a fragment thereof;
   (b) a nucleic acid molecule shown in Table I, application no. 5, column 5 or 8, preferably shown in Table I A, application no. 5, column 5, or in Table I A, application no. 5, column 8, or in Table I B, application no. 5, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 5, preferably shown in Table II A, application no. 5, column 5, or in Table II A, application no. 5, column 8, or in Table II B, application no. 5, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 5, preferably shown in Table I A, application no. 5, column 5, or in Table I A, application no. 5, column 8, or in Table I B, application no. 5, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 5;
   (i) a nucleic acid molecule encoding a polypeptide having the activity represented by a respective protein as depicted in column 5 of Table II, application no. 5;
   (j) a nucleic acid molecule comprising a polynucleotide which is obtained by amplifying a cDNA library or a genomic library using the primers in Table III, application no. 5, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising an expression cassette comprising
[0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 5, column 5 or 8, preferably shown in Table II A, application no. 5, column 5, or in Table II A, application no. 5, column 8, or in Table II B, application no. 5, column 8, or a homolog or a fragment thereof;
   (b) a nucleic acid molecule shown in Table I, application no. 5, column 5 or 8, preferably shown in Table I A, application no. 5, column 5, or in Table I A, application no. 5, column 8, or in Table I B, application no. 5, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 5, preferably shown in Table II A, application no. 5, column 5, or in Table II A, application no. 5, column 8, or in Table II B, application no. 5, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 5, preferably shown in Table I A, application no. 5, column 5, or in Table I A, application no. 5, column 8, or in Table I B, application no. 5, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 5;
   (i) a nucleic acid molecule encoding a polypeptide having the activity represented by a respective protein as depicted in column 5 of Table II, application no. 5;
   (j) a nucleic acid molecule comprising a polynucleotide which is obtained by amplifying a cDNA library or a genomic library using the primers in Table III, application no. 5, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising or being transformed by a vector comprising
[1) a nucleic acid molecule selected from the group consisting of
   (a) a nucleic acid molecule encoding a polypeptide shown in Table II, application no. 5, column 5 or 8, preferably shown in Table II A, application no. 5, column 5, or in Table II A, application no. 5, column 8, or in Table II B, application no. 5, column 8, or a homolog or a fragment thereof;
   (b) a nucleic acid molecule shown in Table I, application no. 5, column 5 or 8, preferably shown in Table I A, application no. 5, column 5, or in Table I A, application no. 5, column 8, or in Table I B, application no. 5, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 5, preferably shown in Table II A, application no. 5, column 5, or in Table II A, application no. 5, column 8, or in Table II B, application no. 5, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 5, preferably shown in Table I A, application no. 5, column 5, or in Table I A, application no. 5, column 8, or in Table I B, application no. 5, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 5;
   (i) a nucleic acid molecule encoding a polypeptide having the activity represented by a respective protein as depicted in column 5 of Table II, application no. 5;
   (j) a nucleic acid molecule comprising a polynucleotide which is obtained by amplifying a cDNA library or a genomic library using the primers in Table III, application no. 5, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 5.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 5 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 5, in the same line as of the respective nucleic acid molecule according to 1) “plastidic” is depicted, or encoding a mitochondrial transit peptide, especially in case in column 6 of Table I, application no. 5, in the same line as of the respective nucleic acid molecule according to 1) “mitochondrial” is depicted.

In another embodiment of said transgenic non-human organism the expression cassette does not comprise 2) a nucleic acid molecule encoding a transit peptide, especially in case in column 6 of Table I, application no. 5, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 5.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 5, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 5, in the same line as of the respective nucleic acid molecule according to 1) “plastidic” is depicted, or encoding a mitochondrial transit peptide, especially in case in column 6 of Table I, application no. 5, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 5, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 5, in the same line as of the respective nucleic acid molecule according to 1) “plastidic” is depicted, or encoding a mitochondrial transit peptide, especially in case in column 6 of Table I, application no. 5, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 5, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 5, in the same line as of the respective nucleic acid molecule according to 1) “plastidic” is depicted, or encoding a mitochondrial transit peptide, especially in case in column 6 of Table I, application no. 5, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 5.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 5, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 5.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 5, in the same line as of the respective nucleic acid molecule according to 1) “plastidic” is depicted, or encoding a mitochondrial transit peptide, especially in case in column 6 of Table I, application no. 5, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 5, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 5, in the same line as of the respective nucleic acid molecule according to 1) “plastidic” is depicted, or encoding a mitochondrial transit peptide, especially in case in column 6 of Table I, application no. 5, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 5 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 5, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 5 (in the same line) and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 5, column 5 or 8, preferably shown in Table II A, application no. 5, column 5, or in Table II A, application no. 5, column 8, or in Table II B, application no. 5, column 8, or a homolog or a fragment thereof;
   (b) a nucleic acid molecule shown in Table I, application no. 5, column 5 or 8, preferably shown in Table I A, application no. 5, column 5, or in Table I A, application no. 5, column 8, or in Table I B, application no. 5, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 5, preferably shown in Table II A, application no. 5, column 5, or in Table II A, application no. 5, column 8, or in Table II B, application no. 5, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 5, preferably shown in Table I A, application no. 5, column 5, or in Table I A, application no. 5, column 8, or in Table I B, application no. 5, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 5;
   (i) a nucleic acid molecule encoding a polypeptide having the activity represented by a respective protein as depicted in column 5 of Table II, application no. 5;
   (j) a nucleic acid molecule comprising a polynucleotide which is obtained by amplifying a cDNA library or a genomic library using the primers in Table III, application no. 5, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising an expression cassette comprising
[0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 5, column 5 or 8, preferably shown in Table II A, application no. 5, column 5, or in Table II A, application no. 5, column 8, or in Table II B, application no. 5, column 8, or a homolog or a fragment thereof;
   (b) a nucleic acid molecule shown in Table I, application no. 5, column 5 or 8, preferably shown in Table I A, application no. 5, column 5, or in Table I A, application no. 5, column 8, or in Table I B, application no. 5, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 5, preferably shown in Table II A, application no. 5, column 5, or in Table II A, application no. 5, column 8, or in Table II B, application no. 5, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 5, preferably shown in Table

I A, application no. 5, column 5, or in Table I A, application no. 5, column 8, or in Table I B, application no. 5, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 5;
(i) a nucleic acid molecule encoding a polypeptide having the activity represented by a respective protein as depicted in column 5 of Table II, application no. 5;
(j) a nucleic acid molecule comprising a polynucleotide which is obtained by amplifying a cDNA library or a genomic library using the primers in Table III, application no. 5, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
or a nucleic acid molecule comprising a sequence which is complementary thereto;]
   and, if desired
2) nucleic acid molecule encoding a transit peptide, preferably encoding a plastidal transit peptide or a mitochondrial transit peptide;
   which are operable linked;]
   or comprising or being transformed by a vector comprising
[1) a nucleic acid molecule selected from the group consisting of
   (a) a nucleic acid molecule encoding a polypeptide shown in Table II, application no. 5, column 5 or 8, preferably shown in Table II A, application no. 5, column 5, or in Table II A, application no. 5, column 8, or in Table II B, application no. 5, column 8, or a homolog or a fragment thereof;
   (b) a nucleic acid molecule shown in Table I, application no. 5, column 5 or 8, preferably shown in Table I A, application no. 5, column 5, or in Table I A, application no. 5, column 8, or in Table I B, application no. 5, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 5, preferably shown in Table II A, application no. 5, column 5, or in Table II A, application no. 5, column 8, or in Table II B, application no. 5, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 5, preferably shown in Table I A, application no. 5, column 5, or in Table I A, application no. 5, column 8, or in Table I B, application no. 5, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 5;
   (i) a nucleic acid molecule encoding a polypeptide having the activity represented by a respective protein as depicted in column 5 of Table II, application no. 5;
   (j) a nucleic acid molecule comprising a polynucleotide which is obtained by amplifying a cDNA library or a genomic library using the primers in Table III, application no. 5, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      whereby, preferably, the nucleic acid molecule according to 1 (a), (b), (c), (d), (e), (f), (g), (h), (i), (j) or (k) distinguishes over the sequence depicted in Table I A and/or I B, application no. 5, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 5, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 5, column 5 or 8, or the coding region thereof, respectively. In a further embodiment the nucleic acid molecule does not encode the polypeptide sequence shown in Table II A and/or II B, application no. 5, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 5, column 5 or 8 and does not encode a protein of the sequence shown in Table II A and/or II B, application no. 5, column 5 or 8. Accordingly, in one embodiment, the protein encoded by a sequence of a nucleic acid according to 1 (a), (b), (c), (d), (e), (f), (g), (h), (i), (j) or (k) does not consist of the sequence shown in Table I A and/or I B, application no. 5, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 5, column 5 or 8 but less than 100%, preferably less than 99.999%, 99.99%, 99.9%, 99%, 98%, 97%, 96% or 95% identical to the sequence shown in Table II A and/or II B, application no. 5, column 5 or 8;
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 5.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 5 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 5, in the same line as of the respective nucleic acid molecule according to 1) “plastidic” is depicted, or encoding a mitochondrial transit peptide, especially in case in column 6 of Table I, application no. 5, in the same line as of the respective nucleic acid molecule according to 1) “mitochondrial” is depicted.

In another embodiment thereof the expression cassette does not comprise 2) a nucleic acid molecule encoding a transit peptide, especially in case in column 6 of Table I, application no. 5, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 5.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the same line in column 5 of Table II, application no. 5, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 5, in the same line as of the respective nucleic acid molecule according to 1) “plastidic” is depicted, or encoding a mitochondrial transit peptide, especially in case in column 6 of Table I, application no. 5, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 5, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 5, in the same line as of the respective nucleic acid molecule according to 1) “plastidic” is depicted, or encoding a mitochondrial transit peptide, especially in case in column 6 of Table I, application no. 5, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 5, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 5, in the same line as of the respective nucleic acid molecule according to 1) “plastidic” is depicted, or encoding a mitochondrial transit peptide, especially in case in column 6 of Table I, application no. 5, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 5.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 5, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 5.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 5, in the same line as of the respective nucleic acid molecule according to 1) “plastidic” is depicted, or encoding a mitochondrial transit peptide, especially in case in column 6 of Table I, application no. 5, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 5, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 5, in the same line as of the respective nucleic acid molecule according to 1) “plastidic” is depicted, or encoding a mitochondrial transit peptide, especially in case in column 6 of Table I, application no. 5, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 5 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 5, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 5 and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

[0291.1.1.5] to [0299.1.1.5] for the disclosure of these paragraphs see [0291.1.1.1] to [0299.1.1.1] above.

The term “biological active portion” or “biological activity” means a polypeptide as depicted in the respective line in Table II, application no. 5, column 3 or a portion of said polypeptide which still has at least 10% or 20%, preferably 30%, 40%, 50% or 60%, especially preferably 70%, 75%, 80%, 90% or 95% of the enzymatic or biological activity of the natural or starting enzyme or protein. [0301.1.1.5] to [0304.1.1.5] for the disclosure of these paragraphs see [0301.1.1.1] to [0304.1.1.1] above.

Synthetic oligonucleotide primers for the amplification, e.g. as shown in the respective line in Table III, application no. 5, column 8, by means of polymerase chain reaction can be generated on the basis of a sequence shown herein, for example the sequence shown in the respective line in Table I, application no. 5, columns 5 or 8, or the sequences derived from Table II, application no. 5, columns 5 or 8, respectively.

Moreover, it is possible to identify a conserved region of a protein from various organism by carrying out protein sequence alignments with the polypeptide encoded by the nucleic acid molecules of the present invention, in particular with the sequences encoded by the nucleic acid molecule shown in the respective line in column 5 or 8 of Table I, application no. 5, from which conserved regions, and in turn, degenerate primers can be derived. Conserved regions are those, which show a very little variation in the amino acid in one particular position of several homologs from different origin. The consensus sequence and polypeptide motifs shown in the respective line in column 8 of Table IV, application no. 5, are derived from such alignments of homologous proteins.

In an embodiment of the present invention, in the method of the present invention the activity of a polypeptide comprising or consisting of a consensus sequence or at least one polypeptide motif shown in the respective line in Table IV, application no. 5, column 8, is increased. In another embodiment thereof, the method of the present invention relates to a polypeptide comprising or consisting of a consensus sequence shown in the respective line in Table IV, application no. 5, column 8 or comprising at least one polypeptide motif shown in the respective line in Table IV, application no. 5, column 8, whereby not more than 20, 15, 10, 9, 8, 7, 6, 5 4, 3, 2 or 1, or 0 of the amino acid positions indicated can be replaced by any amino acid. In another embodiment thereof, the method of the present invention relates to a polypeptide comprising or consisting of a consensus sequence shown in the respective line in Table IV, application no. 5, column 8 or comprising at least one polypeptide motif shown in the respective line in Table IV, application no. 5, column 8, whereby not more than 15%, 10%, 5%, 4%, 3%, 2% or 1%, or 0% of the amino acid positions defined as distinct specific amino acids are/is replaced by another amino acid. In another embodiment thereof, the method of the present invention relates to a polypeptide comprising or consisting of a consensus sequence shown in the respective line in Table IV, application no. 5, column 8 or comprising at least one polypeptide motif shown in the respective line in Table IV, application no. 5, column 8, whereby not more than 20, 15, 10, 9, 8, 7, 6, 5, 4, 3, 2 or 1, or 0 amino acids are inserted into a consensus sequence or polypeptide motif.

In an embodiment of the present invention, in the method of the present invention the activity of a polypeptide comprising or consisting of a consensus sequence or at least one polypeptide motif shown in the respective line in Table IV, application no. 5, column 8, is increased, whereby said polypeptide distinguishes over the sequence depicted in Table II, application no. 5, columns 5 or 8 by one or more amino acids.

In one embodiment, said polypeptide of the invention distinguishes over the sequence shown in the respective line in Table II, application no. 5, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II B, application no. 5, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in Table II, application no. 5, columns 5 or 8.

[0309.1.1.5] to [0321.1.1.5] for the disclosure of these paragraphs see [0309.1.1.1] to [0321.1.1.1] above.

Polypeptides having above-mentioned activity, i.e. conferring the production or the increased production of the fine chemical tryptophan as compared to a corresponding, e.g.

non-transformed, wild type non-human organism, like a microorganism or a plant cell, plant or part thereof, can be encoded by other DNA sequences which hybridize to the sequences shown in the respective line in Table I, application no. 5, columns 5 and 8, preferably the coding region thereof, at least under relaxed hybridization conditions and which encode the expression of polypeptides conferring the production or the increased production of the respective fine chemical tryptophan as compared to a corresponding, e.g. non-transformed, wild type non-human organism, like a microorganism or a plant cell, plant or part thereof.

[0323.1.1.5] to [0329.1.1.5] for the disclosure of these paragraphs see [0323.1.1.1] to [0329.1.1.1] above.

Further, the nucleic acid molecule of the invention comprises a nucleic acid molecule, which is a complement of one of the nucleotide sequences of above-mentioned nucleic acid molecules or a portion thereof. A nucleic acid molecule or its sequence which is complementary to one of the nucleotide molecules or sequences shown in the respective line in Table I, application no. 5, columns 5 or 8, preferably the coding region thereof, homologs or fragments thereof, is one which is sufficiently complementary to one of the nucleotide molecules or sequences shown in the respective line in Table I, application no. 5, columns 5 or 8, preferably the coding region thereof, homologs or fragments thereof, such that it can hybridize to one of the nucleotide sequences shown in Table I, application no. 5, columns 5 or 8, preferably the coding region thereof, homologs or fragments thereof, thereby forming a stable duplex. Preferably, the hybridization is performed under stringent hybridization conditions. However, a complement of one of the herein disclosed sequences is preferably a sequence complement thereto according to the base pairing of nucleic acid molecules well known to the skilled person. For example, the bases A and G undergo base pairing with the bases T and U or C, resp. and visa versa. Modifications of the bases can influence the base-pairing partner.

The nucleic acid molecule of the invention comprises a nucleotide sequence which is at least about 30%, 35%, 40%, 45%, 50%, 55%, 60%, 65%, 70%, 75%, 80%, 90%, 95%, 96%, 97%, 98%, 99%, 99.5% or more homologous to a nucleotide sequence shown in Table I, application no. 5, columns 5 or 8, preferably the coding region thereof, homologs or fragments thereof, and preferably has above-mentioned activity, in particular conferring the production or the increased production of the fine chemical tryptophan, respectively, after increasing the activity or an activity of a gene as shown in the respective line in Table I or of a gene product, e.g. as shown in the respective line in Table II, application no. 5, column 5 or 8, by for example in one embodiment expression either in the cytosol or in an organelle such as a plastid or mitochondria or both, preferably in a plastid, or in another embodiment non-targeted or targeted.

The nucleic acid molecule of the invention comprises a nucleotide sequence or molecule which hybridizes, preferably hybridizes under stringent conditions as defined herein, to one of the nucleic acid molecule shown in the respective line in Table I, preferably Table IB, application no. 5, columns 5 or 8, preferably the coding region thereof, fragments or homologs thereof, and encodes a protein having above-mentioned activity, e.g. conferring the production or the increased production of the respective fine chemical tryptophan as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted, and optionally, the activity thereof is selected from the group consisting of 3-deoxy-7-phosphoheptulonate synthase, 3-ketoacyl-CoA thiolase, acetyltransferase, aldehyde dehydrogenase, amino acid transporter, aromatic amino acid aminotransferase, At1g19800-protein, At2g33510-protein, At4g32480-protein, At5g16650-protein, at5g21910-protein, ATP synthase subunit E, ATP-binding component of a transport system, auxin response factor, b1163-protein, b1346-protein, b1522-protein, b1796-protein, b2739-protein, b2849-protein, b3989-protein, b4029-protein, b4121-protein, binding-protein-dependent transport systems inner membrane component, branched-chain amino acid permease, calcium-dependent protein kinase, carbon dioxide concentrating mechanism protein, cardiolipin synthase, CBL-interacting protein kinase, Chaperone protein CIpB, choline kinase, coproporphyrinogen III oxidase, cullin, cyclin, cyclin D , cystathionine gamma-synthase, DNA mismatch repair protein, electron transfer flavoprotein subunit beta, elongation factor Tu, enoyl-CoA hydratase, eukaryotic translation initiation factor, eukaryotic translation initiation factor 5, fimbrial-like protein, flavodoxin, geranylgeranyl pyrophosphate synthase, gibberellin 20-oxidase, glucose dehydrogenase, glucose-6-phosphate 1-dehydrogenase, glutamate-ammonia-ligase, glutamine synthetase, glutaredoxin, glycerol kinase, glycosyl transferase, harpin-induced family protein, histone acetyltransferase, hydrolase, imidazole glycerol phosphate synthase subunit, isochorismate synthase, isopentenyl diphosphate isomerase, Leucyl/phenylalanyl-tRNA-protein transferase, lipase, L-ribulose-5-phosphate 4-epimerase, malate dehydrogenase, malic enzyme, methyl-accepting chemotaxis protein, methyltransferase, monthiol glutaredoxin, NAD(P)H-quinone oxidoreductase subunit, nitrate/nitrite transport protein, photosystem II protein, polygalacturonase, precorrin methylase, protein kinase, protein phosphatase, putative transport system permease protein, pyruvate kinase, recombinase A, RNA binding protein, RNA-binding protein, Sec-independent protein translocase subunit, short-chain alcohol dehydrogenase family, slr1107-protein, threonine synthase, transaldolase, transcription factor, transcriptional regulator, transport protein, trehalose-phosphatase, tryptophan biosynthesis protein, yfl054c-protein, and Zm_{—}4842_BE510522-protein, respectively.

Moreover, the nucleic acid molecule of the invention can comprise only a portion of the coding region of one of the nucleic acid molecules depicted in Table I, application no. 5, columns 5 or 8, for example a fragment which can be used as a probe or primer or a fragment encoding a biologically active portion of the polypeptide of the present invention or of a polypeptide used in the process of the present invention, i.e. having above-mentioned activity, e.g. conferring the production or the increased production of the respective fine chemical tryptophan as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted. The nucleotide sequences determined from the cloning of the present protein-according-to-the-invention-encoding gene allows for the generation of probes and primers designed for use in identifying and/or cloning its homologues from other cell types and organisms. The probe/primer typically comprises a substantially purified oligonucleotide. The oligonucleotide typically comprises a region of nucleotide sequence that hybridizes under stringent conditions to at least about 12, 15 preferably about 20 or 25, more preferably about 40, 50 or 75 consecutive nucleotides of a sense strand of one of the sequences set forth, e.g., in the respective line in Table I, application no. 5, column 5 or 8, preferably the coding region thereof, or a homolog or a fragment thereof, an anti-sense sequence of one of the sequences, e.g., set forth in the respective line in Table I, application no. 5, columns 5 or 8, preferably the coding region thereof, or a homolog or a fragment thereof or naturally occurring mutants thereof. Primers based on a nucleic acid molecule of invention can be used in PCR reactions to clone homologs of the polypeptide of the invention or of the polypeptide used in the process of the invention, e.g. as the primers described in the examples of the present invention, e.g. as shown in the examples. A PCR with the primers shown in the respective line in Table III, column 8 will result in a fragment of the gene product as shown in Table II, application no. 5, column 5.

Primer sets are interchangeable. The person skilled in the art knows how to design and combine said primers to result in the desired product, e.g. in a full length clone or a partial sequence. Probes based on the sequences of the nucleic acid molecule of the invention or used in the process of the present invention can be used to detect transcripts or genomic sequences encoding the same or homologous proteins. The probe can further comprise a label group attached thereto, e.g. the label group can be a radioisotope, a fluorescent compound, an enzyme, or an enzyme co-factor. Such probes can be used as a part of a genomic marker test kit for identifying cells which express a polypeptide of the invention or used in the process of the present invention, such as by measuring a level of an encoding nucleic acid molecule in a sample of cells, e.g. detecting mRNA levels or determining, whether a genomic gene comprising the sequence of the polynucleotide of the invention or used in the processes of the present invention has been mutated or deleted.

As the expression of a respective nucleic acids of the invention is related to the synthesis of the respective fine chemical tryptophan its function as a probe extends to the detection of microorganisms, plant tissues, plants, plant variets, plant ecotypes or plant genera with varying capability or potential for synthesis of the respective fine chemical tryptophan. Therefore in one embodiment the present invention relates to a method for analyzing the capability or potential of a plant tissue, a plant, a plant variety or plant ecotype to produce the fine chemical tryptophan by using the nucleic acid of the invention or parts thereof as a probe to detect the amount of the nucleic acid of the invention in the non-human organism or a part thereof in comparison to another non-human organism.

The nucleic acid molecule of the invention encodes a polypeptide or portion thereof which includes an amino acid sequence which is sufficiently homologous to the amino acid sequence shown in respective line in Table II, application no. 5, columns 5 or 8 such that the protein or portion thereof maintains the ability to confer the production or the increased production of the respective fine chemical tryptophan as compared to a corresponding, e.g. non-transformed, wild type non-human organism, like a microorganism or a plant cell, plant or part thereof, in particular increasing the activity as mentioned above or as described in the examples in microorganisms or plants.

As used herein, the language “sufficiently homologous” refers to proteins or portions thereof which have amino acid sequences which include a minimum number of identical or equivalent amino acids (e.g. an amino acid which has a similar side chain as an amino acid in one of the sequences of the polypeptide of the present invention) to an amino acid sequence shown in the respective line in Table II, application no. 5, columns 5 or 8 such that the protein or portion thereof is able to confer the production or the increased production of the respective fine chemical tryptophan as compared to a corresponding, e.g. non-transformed, wild type non-human organism, like a microorganism or a plant cell, plant or part thereof. For example having the activity of a protein as shown in the respective line in Table II, application no. 5, column 3 and as described herein.

In an embodiment of the present invention, the nucleic acid molecule of the present invention comprises a nucleic acid molecule that encodes a portion of the protein of the present invention. The protein is at least about 30%, 35%, 40%, 45% 50%, 55%, 60%, 65%, 70%, 75%, 80%, 85%, 90%, 91%, 92%, 93%, 94%, 95%, 96%, 97%, 98%, 99%, 99.5% or more homologous to an entire amino acid sequence shown in the respective line of Table II, application no. 5, column 5 or 8 and has the above-mentioned activity, e.g. conferring the production or the increased production of the respective fine chemical tryptophan as compared to a corresponding, e.g. non-transformed, wild type non-human organism, like a microorganism or a plant cell, plant or part thereof by, for example in a embodiment expression either in the cytosol or in an organelle such as a plastid or mitochondria or both, preferably in a plastid, or in another embodiment by targeted or non-targeted expression.

[0338.1.1.5] to [0339.1.1.5] for the disclosure of these paragraphs see [0338.1.1.1] to [0339.1.1.1] above.

The invention further relates to nucleic acid molecules or methods using said nucleic acid molecules that differ from one of the nucleotide sequences shown in the respective line in Table I, application no. 5, columns 5 or 8, preferably the coding region thereof, or fragments or homologs thereof, due to degeneracy of the genetic code but encode a polypeptide of the present invention, in particular a polypeptide having the above-mentioned activity, e.g. as those polypeptides depicted in the respective line in Table II, application no. 5, columns 5 or 8, fragments or homologs thereof. Advantageously, the nucleic acid molecule of the invention comprises, or in an other embodiment has, a nucleotide sequence encoding a protein comprising, or in an other embodiment having, an amino acid sequence shown in the respective line in Table II, application no. 5, columns 5 or 8, fragments or homologs thereof. In a further embodiment, the nucleic acid molecule of the invention encodes a full length protein which is substantially homologous to an amino acid sequence shown in the respective line in Table II, application no. 5, columns 5 or 8, or homologs thereof. However, in an embodiment, the nucleic acid molecule of the present invention does not consist of the sequence shown in the respective line in Table I, preferably Table IA, application no. 5, columns 5 or 8, preferably the coding region thereof, but the functional homologs thereof.

[0341.1.1.5] to [0343.1.1.5] for the disclosure of these paragraphs see [0341.1.1.1] to [0343.1.1.1] above.

Accordingly, in another embodiment, a nucleic acid molecule of the invention is at least 15, 20, 25 or 30 nucleotides in length. Preferably, it hybridizes under stringent conditions to a nucleic acid molecule comprising a nucleotide sequence of the nucleic acid molecule of the present invention or used in the process of the present invention, e.g. comprising the sequence shown in the respective line in Table I, application no. 5, columns 5 or 8, preferably the coding region thereof. In another embodiment the nucleic acid molecule is preferably at least 20, 30, 50, 100, 250 or more nucleotides in length.

for the disclosure of this paragraph see [0345.1.1.1] above.

Preferably, a nucleic acid molecule of the invention that hybridizes under stringent conditions to a sequence shown in the respective line in Table I, application no. 5, columns 5 or 8, preferably the coding region thereof, corresponds to a naturally-occurring nucleic acid molecule of the invention. As used herein, a “naturally-occurring” nucleic acid molecule refers to a RNA or DNA molecule having a nucleotide sequence that occurs in nature (e.g. encodes a natural protein). Preferably, the nucleic acid molecule encodes a natural protein having above-mentioned activity, e.g. conferring the production or the increased production of the respective fine chemical tryptophan as compared to a corresponding, e.g. non-transformed, wild type non-human organism, like a microorganism or a plant cell, plant or part thereof after increasing the expression or activity thereof or the activity of a protein of the invention or used in the process of the invention, in an embodiment for example expression either in the cytosol or in an organelle such as a plastid or mitochondria, preferably in plastids, or, in another embodiment by targeted or non-targeted expression.

for the disclosure of this paragraph see [0347.1.1.1] above [0348.1.5.5] For example, nucleotide substitutions leading to amino acid substitutions at “non-essential” amino acid residues can be made in a sequence of the nucleic acid molecule of the invention or used in the process of the invention, e.g. shown in the respective line in Table I, application no. 5, columns 5 or 8, preferably the coding region thereof, or fragments or homologs thereof.

[0349.1.1.5] to [0350.1.1.5] for the disclosure of these paragraphs see [0349.1.1.1] to 0350.1.1.1] above.

Accordingly, the invention relates to nucleic acid molecules encoding a polypeptide having above-mentioned activity, in a non-human organisms or parts thereof by for example expression either in the cytosol or in an organelle such as a plastid or mitochondria or both, preferably in plastids, that contain changes in amino acid residues that are not essential for said activity. Such polypeptides differ in amino acid sequence from a sequence depicted in the respective line in Table II, especially Table IIA, application no. 5, columns 5 or 8, but retain said activity described herein. The nucleic acid molecule can comprise a nucleotide sequence encoding a polypeptide, wherein the polypeptide comprises an amino acid sequence at least about 50% identical to an amino acid sequence shown in the respective line in Table II, application no. 5, columns 5 or 8, and is capable of conferring the production or the increased production of the respective fine chemical tryptophan as compared to a corresponding, e.g. non-transformed, wild type non-human organism, like a microorganism or a plant cell, plant or part thereof, after increasing its activity for example in an embodiment by expression either in the cytosol or in an organelle such as a plastid or mitochondria or both, preferably in plastids, or, in another embodiment by targeted or non-targeted expression. Preferably, the protein encoded by the nucleic acid molecule is at least about 60%, 70%, 80%, 90%, 95%, 96%, 97%, 98%, 99% or 99.5% identical to the sequence shown in the respective line in Table II, application no. 5, columns 5 or 8.

[0352.1.1.5] to [0357.1.1.5] for the disclosure of these paragraphs see [0352.1.1.1] to [0357.1.1.1] above.

Functional equivalents of the nucleic acid molecules according to the present invention and/or used in the process according to the present invention represent an embodiment of homologs. Functional equivalents derived from the nucleic acid molecules as shown in the respective line in Table I, application no. 5, columns 5 or 8, preferably the coding region thereof, according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the nucleic acid molecules as shown in the respective line in Table I, application no. 5, columns 5 or 8, preferably the coding region thereof, according to the invention and encode polypeptides having essentially the same properties as the polypeptide as shown in the respective line in Table II, application no. 5, columns 5 or 8.

Functional equivalents derived from one of the polypeptides as shown in the respective line in Table II, application no. 5, columns 5 or 8 according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the polypeptides as shown in the respective line in Table II, application no. 5, columns 5 or 8 according to the invention and having essentially the same properties as the polypeptide as shown in the respective line in Table II, application no. 5, columns 5 or 8.

for the disclosure of this paragraph see [0359.1.1.1] above [0360.1.5.5] A nucleic acid molecule encoding a homolog to a polypeptide depicted in the respective line in Table II, application no. 5, columns 5 or 8 can be created by introducing one or more nucleotide substitutions, additions or deletions into the nucleic acid molecule of the present invention, in particular of Table I, application no. 5, columns 5 or 8, in the respective line, such that one or more amino acid substitutions, additions or deletions are introduced into the encoded protein. Mutations can be introduced into the nucleic acid molecules as depicted in the respective line in Table I, application no. 5, columns 5 or 8, preferably the coding region thereof, by standard techniques, such as site-directed mutagenesis, PCR-mediated mutagenesis or other methods known to the person skilled in the art.

[0361.1.1.5] to [0363.1.1.5] for the disclosure of these paragraphs see [0361.1.1.1] to [0363.1.1.1] above.

Homologues of the nucleic acid sequences used, with the sequences shown in the respective line in Table I, application no. 5, columns 5 or 8, preferably the coding region thereof, comprise also allelic variants with at least approximately 30%, 35%, 40%, 45%, 50%, 55%, 60%, 65%, 70%, 75%, 80%, 85%, 90%, 91%, 92%, 93%, 94% 95%. 96%, 97%, 98%, 99%, 99.5% or more % homology with one of the nucleotide sequences shown or the above-mentioned derived nucleic acid sequences or their homologues or derivatives or parts of these. Allelic variants encompass in particular functional variants which can be obtained by deletion, insertion or substitution of nucleotides from the sequences shown in the respective line, preferably in Table I, columns 5 or 8, preferably the coding region thereof, or from the derived nucleic acid sequences, the intention being, however, that the enzymatic activity or the biological activity of the resulting proteins synthesized is advantageously retained or increased.

In one embodiment of the present invention, the nucleic acid molecule of the invention or used in the process of the invention comprises the sequences shown in the respective line in any of the Table I, application no. 5, columns 5 or 8, preferably the coding region thereof, fragments or homologs thereof. It is preferred that the nucleic acid molecule comprises as little as possible other nucleotides not shown in the respective line in any one of Table I, application no. 5, columns 5 or 8, preferably the coding region thereof, fragments or homologs thereof. In one embodiment, the nucleic acid molecule comprises less than 500, 400, 300, 200, 100, 90, 80, 70, 60, 50 or 40 further nucleotides. In a further embodiment, the nucleic acid molecule comprises less than 30, 20 or 10 further nucleotides. In one embodiment, the nucleic acid molecule used in the process of the invention is identical to the sequences shown in the respective line in Table I, application no. 5, columns 5 or 8, preferably the coding region thereof.

Also preferred is that the nucleic acid molecule used in the process of the invention encodes a polypeptide comprising the sequence shown in the respective line in Table II, application no. 5, columns 5 or 8, fragments or homologs thereof. In one embodiment, the nucleic acid molecule encodes less than 150, 130, 100, 80, 60, 50, 40 or 30 further amino acids. In a further embodiment, the encoded polypeptide comprises less than 20, 15, 10, 9, 8, 7, 6 or 5 further amino acids. In one embodiment used in the inventive process, the encoded polypeptide is identical to the sequences shown in the respective line in Table II, application no. 5, columns 5 or 8.

In one embodiment, the nucleic acid molecule of the invention or used in the process encodes a polypeptide comprising the sequence shown in the respective line in Table II, application no. 5, columns 5 or 8, or fragments or homologs thereof, and comprises less than 100 further nucleotides. In a further embodiment, said nucleic acid molecule comprises less than 30 further nucleotides. In one embodiment, the nucleic acid molecule used in the process is identical to a coding sequence of the sequences shown in the respective line in Table I, application no. 5, columns 5 or 8.

Polypeptides (=proteins), which still have the essential biological or enzymatic activity of the polypeptide of the present invention conferring the production or the increased production of the respective fine chemical tryptophan as compared to a corresponding, e.g.

non-transformed, wild type non-human organism, like a microorganism or a plant cell, plant or part thereof, i.e. whose activity is essentially not reduced, are polypeptides with at least 10%, 20%, 30%, 40%, 50%, 60%, 70%, 80%, 90%, 95% or more of the wild type biological activity or enzymatic activity, advantageously, the activity is essentially not reduced in comparison with the activity of a polypeptide shown in the respective line in Table II, application no. 5, columns 5 or 8 expressed under identical conditions.

Homologues of nucleic acid molecules shown in the respective line in Table I, application no. 5, columns 5 or 8, preferably the coding region thereof, or fragments thereof, or of the derived sequences shown in the respective line in Table II, application no. 5, columns 5 or 8, or fragments thereof, also mean truncated sequences, cDNA, single-stranded DNA or RNA of the coding and noncoding DNA sequence. Homologues of said sequences are also understood as meaning derivatives, which comprise noncoding regions such as, for example, UTRs, introns, terminators, enhancers or promoter variants. The promoters upstream of the nucleotide sequences stated can be modified by one or more nucleotide substitution(s), insertion(s) and/or deletion(s) without, however, interfering with the functionality or activity either of the promoters, the open reading frame (=ORF) or with the 3′-regulatory region such as terminators or other 3′-regulatory regions, which are far away from the ORF. It is furthermore possible that the activity of the promoters is increased by modification of their sequence, or that they are replaced completely by more active promoters, even promoters from heterologous organisms. Appropriate promoters are known to the person skilled in the art and are mentioned herein below.

[0370.1.1.5] to [0379.1.1.1] for the disclosure of these paragraphs see [0370.1.1.1] to [0379.1.1.1] above.

Moreover, a native polypeptide conferring the increase of the respective fine chemical tryptophan in a non-human organism or a part thereof can be isolated from cells (e.g., endothelial cells), for example using the antibody of the present invention as described below, in particular, an antibody against proteins having 3-deoxy-7-phosphoheptulonate synthase, 3-ketoacyl-CoA thiolase, acetyltransferase, aldehyde dehydrogenase, amino acid transporter, aromatic amino acid aminotransferase, At1g19800-protein, At2g33510-protein, At4g32480-protein, At5g16650-protein, at5g21910-protein, ATP synthase subunit E, ATP-binding component of a transport system, auxin response factor, b1163-protein, b1346-protein, b1522-protein, b1796-protein, b2739-protein, b2849-protein, b3989-protein, b4029-protein, b4121-protein, binding-protein-dependent transport systems inner membrane component, branched-chain amino acid permease, calcium-dependent protein kinase, carbon dioxide concentrating mechanism protein, cardiolipin synthase, CBL-interacting protein kinase, Chaperone protein CIpB, choline kinase, coproporphyrinogen III oxidase, cullin, cyclin, cyclin D , cystathionine gamma-synthase, DNA mismatch repair protein, electron transfer flavoprotein subunit beta, elongation factor Tu, enoyl-CoA hydratase, eukaryotic translation initiation factor, eukaryotic translation initiation factor 5, fimbrial-like protein, flavodoxin, geranylgeranyl pyrophosphate synthase, gibberellin 20-oxidase, glucose dehydrogenase, glucose-6-phosphate 1-dehydrogenase, glutamate-ammonia-ligase, glutamine synthetase, glutaredoxin, glycerol kinase, glycosyl transferase, harpin-induced family protein, histone acetyltransferase, hydrolase, imidazole glycerol phosphate synthase subunit, isochorismate synthase, isopentenyl diphosphate isomerase, Leucyl/phenylalanyl-tRNA-protein transferase, lipase, L-ribulose-5-phosphate 4-epimerase, malate dehydrogenase, malic enzyme, methyl-accepting chemotaxis protein, methyltransferase, monthiol glutaredoxin, NAD(P)H-quinone oxidoreductase subunit, nitrate/nitrite transport protein, photosystem II protein, polygalacturonase, precorrin methylase, protein kinase, protein phosphatase, putative transport system permease protein, pyruvate kinase, recombinase A, RNA binding protein, RNA-binding protein, Sec-independent protein translocase subunit, shortchain alcohol dehydrogenase family, slr1107-protein, threonine synthase, transaldolase, transcription factor, transcriptional regulator, transport protein, trehalose-phosphatase, tryptophan biosynthesis protein, yfl054c-protein, or Zm_{—}4842_BE510522-protein activity, respectively, or an antibody against polypeptides as shown in the respective line in Table II, application no. 5, columns 5 or 8, or fragments or homologs thereof which can be produced by standard techniques utilizing the polypeptid of the present invention or fragment thereof, i.e., the polypeptide of this invention (FCRP). Preferred are monoclonal antibodies.

for the disclosure of this paragraph see [0381.1.1.1] above.

In one embodiment, the present invention relates to a polypeptide as depicted in the respective line in Table II, especially in Table IIA or especially in Table IIB, application no. 5, columns 5 or 8, or fragment or homolog thereof or as coded by the nucleic acid molecule shown in the respective line in Table I, especially Table IA or especially Table IB, application no. 5, columns 5 or 8, preferably the coding region thereof, or fragment or homolog thereof.

In an embodiment, the present invention relates to a polypeptide comprising or consisting of a consensus sequence shown in the respective line in Table IV, application no. 5, column 8 or at least one polypeptide motif shown in the respective line in Table IV, application no. 5, column 8.

In an embodiment thereof, the present invention relates to a polypeptide comprising or consisting of a consensus sequence shown in the respective line in Table IV, application no. 5, column 8 or comprising at least one polypeptide motif shown in the respective line in Table IV, application no. 5, column 8, whereby not more than 20, 15, 10, 9, 8, 7, 6, 5, 4, 3, 2, or 1, or 0 of the amino acids can be replaced by any amino acid.

In another embodiment thereof, the present invention relates to a polypeptide comprising or consisting of a consensus sequence shown in the respective line in Table IV, application no. 5, column 8 or comprising at least one polypeptide motif shown in the respective line in Table IV, application no. 5, column 8, whereby not more than 15%, 10%, 5%, 4%, 3%, 2% or1% or 0% of the amino acids defined as distinct specific amino acids are/is replaced by another amino acid.

In another embodiment thereof, the present invention relates to a polypeptide comprising or consisting of a consensus sequence shown in the respective line in Table IV, application no. 5, column 8 or comprising at least one polypeptide motif shown in the respective line in Table IV, application no. 5, column 8, whereby not more than 20, 15, 10, 9, 8, 7, 6, 5, 4, 3, 2, or 1, or 0 amino acids are inserted into the consensus sequence or polypeptide motif.

In an embodiment of the present invention, the polypeptide of the present invention comprises or consists of a consensus sequence or at least one polypeptide motif shown in the respective line in Table IV, application no. 5, column 8, whereby said polypeptide distinguishes over the sequence depicted in the respective line in Table II, application no. 5, columns 5 or 8 by one or more amino acids. In one embodiment, said polypeptide of the invention distinguishes over the sequence shown in the respective line in Table II, application no. 5, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 5, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in the respective line in Table II, application no. 5, columns 5 and 8.

In an embodiment, the invention relates to a polypeptide conferring an increase in the respective fine chemical tryptophan in a non-human organism, especially a microorganism or a plant, or a part thereof, being encoded by the nucleic acid molecule of the invention or used in the process of the invention and having a sequence which distinguishes over the sequence as shown in the respective line in Table II, application no. 5, columns 5 or 8 by one or more amino acids (but not exceeding 5%, preferably 4%, 3%, 2%, 1%, 0.5%, 0.2% or 0.1%). In an embodiment, said polypeptide of the invention does not comprise or consist of the sequence shown in the respective line in Table II, application no. 5, columns 5 or 8. In an embodiment, said polypeptide of the present invention is less than 100%, 99.999%, 99.99%, 99.9% or 99% identical. In one embodiment, said polypeptide which differs at least in one or more amino acids (but not exceeding 5%, preferably 4%, 3%, 2%, 1%, 0.5%, 0.2% or 0.1%) from the polypeptide shown in the respective line in Table II, application no. 5, columns 5 and 8 does not comprise a protein of the sequence shown in the respective line in Table II A and/or II B, application no. 5, columns 5 or 8.

In an embodiment, the present invention relates to a polypeptide having the activity of the protein as shown in the respective line in Table II, application no. 5, column 3, which distinguishes over the sequence depicted in the respective line in Table II, application no. 5, columns 5 or 8 by one or more amino acids, preferably by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 5, columns 5 and 8 by not more than 80%, 70%, 60%, 50%, 40%, 30%, 20%, 10%, 5%, 4%, 3%, 2%, 1% or 0.5%.

In another embodiment the polypeptide of the invention takes the form of a preprotein consisting of a plastidial or mitochondrial transit peptide joint to a polypeptide having the activity of the protein as shown in Table II, column 3, from which the transit peptide is preferably cleaved off upon transport of the preprotein into the organelle for example into the plastid or mitochondrion.

In another embodiment the polypeptide of the invention takes not the form of a preprotein.

[0390.1.1.5] to [0391.1.1.5] for the disclosure of these paragraphs see [0390.1.1.1] to [0391.1.1.1] above.

A polypeptide of the invention can participate in the process of the present invention. The polypeptide or a portion thereof comprises preferably an amino acid sequence, which is sufficiently homologous to an amino acid sequence shown in the respective line in Table II A and/or II B, application no. 5, columns 5 or 8.

Further, the polypeptide can have an amino acid sequence which is encoded by a nucleotide sequence which hybridizes, preferably hybridizes under stringent conditions as described above, to a nucleotide sequence of the nucleic acid molecule of the present invention. Accordingly, the polypeptide has an amino acid sequence which is encoded by a nucleotide sequence that is at least about 35%, 40%, 45%, 50%, 55%, 60%, 65%, 70%, 75%, 80%, 85%, 90%, 91%, 92%, 93%, 94%, 95%, 96%, 97%, 98%, 99%, 99.5% or more homologous to one of the amino acid sequences as shown in the respective line in Table II A and/or II B, application no. 5, columns 5 or 8, or fragments thereof. The preferred polypeptide of the present invention preferably possesses at least one of the activities according to the invention and described herein. A preferred polypeptide of the present invention includes an amino acid sequence encoded by a nucleotide sequence which hybridizes, preferably hybridizes under stringent conditions, to a nucleotide sequence shown in the respective line in Table I A and/or I B, application no. 5, columns 5 or 8, preferably the coding region thereof, or fragments thereto or which is homologous thereto, as defined above.

The polypeptide of the present invention can vary from the sequences shown in the respective line in Table II A and/or II B, application no. 5, columns 5 or 8 in amino acid sequence due to natural variation or mutagenesis, as described in detail herein. Accordingly, the polypeptide comprise an amino acid sequence which is at least about 35%, 40%, 45%, 50%, 55%, 60%, 65%, 70%, 75%, 80%, 85% or 90, 91%, 92%, 93%, 94%, 95%, 96%, 97%, 98%, 99%, 99.5% or more homologous to an entire amino acid sequence shown in the respective line in Table II A and/or II B, application no. 5, columns 5 or 8.

Biologically active portions of a polypeptide of the present invention include peptides comprising amino acid sequences derived from the amino acid sequence of the polypeptide of the present invention or used in the process of the present invention, e.g., the amino acid sequence shown in the respective line in Table II, application no. 5, columns 5 or 8 or the amino acid sequence of a protein homologous thereto, which include fewer amino acids than a full length polypeptide of the present invention or used in the process of the present invention or the full length protein which is homologous to a polypeptide of the present invention or used in the process of the present invention depicted herein, and exhibit at least one activity of a polypeptide of the present invention or used in the process of the present invention.

for the disclosure of this paragraph see [0396.1.1.1] above.

Manipulation of the nucleic acid molecule of the invention may result in the production of a protein having differences from the protein as shown in the respective line in Table II, application no. 5, column 5 or 8. Differences shall mean at least one amino acid different from the sequences as shown in the respective line in Table II, application no. 5, column 5 or 8, preferably at least 2, 3, 4, 5, 6, 7, 8, 9 or 10 amino acids (especially but not exceeding 0.1%, preferably 0.2%, 0.5%, 1%, 2%, 3% 4% or 5%), more preferably at least 15, 20, 25, 30, 35, 40, 45 or 50 amino acids (especially but not exceeding 0.5%, preferably 1%, 2%, 3% 4%, 5%, 10% or 25%) different from the sequences as shown in the respective line in Table II, application no. 5, column 5 or 8. These proteins may be improved in efficiency or activity, or may be more stable and therefore present in greater numbers in the cell as compared to the wild-type cell.

Any mutagenesis strategies for the polypeptide of the present invention or the polypeptide used in the process of the present invention to result in increasing said activity are not meant to be limiting; variations on these strategies will be readily apparent to one skilled in the art. Using such strategies, and incorporating the mechanisms disclosed herein, the nucleic acid molecule and polypeptide of the invention may be utilized to generate algae, ciliates, fungi, other microorganisms like C. glutamicum or plants or parts thereof, expressing proteins as shown in the respective line in Table II, application no. 5, column 5 or 8, or mutated proteins thereof. The nucleic acid molecules and polypeptide molecules of the invention are expressed such that the yield, production, and/or efficiency of production of a desired compound is improved.

[0399.1.1.5] to [0401.1.1.5] for the disclosure of these paragraph see [0399.1.1.1] to [0401.1.1.1] above.

In one embodiment, a protein (=polypeptide) as shown in the respective line in Table II, application no. 5, column 5 or 8 refers to a polypeptide having an amino acid sequence corresponding to the polypeptide of the invention, especially to a polypeptide as shown in Table II, application no. 5, columns 5 or 8 or homologs thereof, or used in the process of the invention, whereas a “non- inventive protein or polypeptide” or “other polypeptide” refers to a polypeptide having an amino acid sequence corresponding to a protein which is not substantially homologous to a polypeptide of the invention, preferably which is not substantially homologous to a polypeptide or protein as shown in the respective line in Table II, application no. 5, column 5 or 8 or homologs thereof, e.g., a protein which does not confer the activity described herein and which is derived from the same or a different organism.

[0403.1.1.5] to [0409.1.1.5] for the disclosure of these paragraphs see [0403.1.1.1] to [0409.1.1.1] above.

As the nucleic acid molecules of the invention and/or the expression thereof is related to the synthesis of the respective fine chemical tryptophan its function as a probe extends to the detection of microorganisms, plant tissues, plants, plant variets, plant ecotypes or plant genera with varying, advantageously increased, capability or potential for synthesis of the respective fine chemical tryptophan. Therefore in one embodiment the present invention relates to a method for analyzing the capability or potential of a plant tissue, a plant, a plant variety or ecotype to produce the respective fine chemical tryptophan by using the respective antibody of the invention as a probe to detect the amount of the polypeptide encoded by said nucleic acid molecule of the invention in a non-human organism in comparison to another organism.

Therefore in one embodiment the invention relates to a method to probe a non-human organism for its capability for the production of the respective fine chemical comprising the steps of
a) analyzing the sequence and/or the expression of the respective nucleic acid molecule of the invention in least two non-human organisms which differ in the capability to produce the respective fine chemical;
b) associate the different sequence and/or expression level of the nucleic acid molecules of the invention with their different capability to produce the respective fine chemical;
c) analyze the sequence and/or the expression of the nucleic acid molecule of the invention in at least a third non-human organism;
d) predict the capability of the at least third organism to produce the respective fine chemical based by comparing the sequence and/or expression of the nucleic acid of the invention to the sequences and/or expression levels of the nucleic acid molecule of the invention of the at least two organisms analyzed in step a.

[0411.1.1.5] to [0430.1.1.5] for the disclosure of these paragraphs see [0411.1.1.1] to [0430.1.1.1] above.

Accordingly the present invention relates to any cell transgenic for any nucleic acid characterized as part of the invention, e.g. conferring the increase of the respective fine chemical tryptophan in a cell or a non-human organism or a part thereof, e.g. the nucleic acid molecule of the invention, the nucleic acid construct of the invention, the vector of the invention, the expression cassette according to the invention, or a nucleic acid molecule encoding the polypeptide of the invention, e.g. encoding a polypeptide having an activity as the protein as shown in the respective line in Table II, application no. 5, column 3. Due to the above-mentioned activity the respective fine chemical tryptophan content in a cell or a non-human organism is increased. For example, due to modulation or manipulation, the cellular activity is increased, in a preferred embodiment in organelles such as plastids or mitochondria, e.g. due to an increased expression or specific activity or specific targeting of the subject matters of the invention in a cell or a non-human organism or a part thereof especially in organelles such as plastids or mitochondria, or in another embodiment in the cytosol. Transgenic for a polypeptide having a protein or a protein activity means herein that due to modulation or manipulation of the genome, the activity of protein as shown in the respective line in Table II, application no. 5, column 3 or a protein as shown in the respective line in Table II, application no. 5, column 3-like activity is increased in the cell or non-human organism or part thereof, especially in organelles such as plastids or mitochondria, or especially in the cytosol. Examples are described above in context with the process of the invention.

for the disclosure of this paragraph see [0432.1.1.1] above.

A naturally occurring expression cassette - for example the naturally occurring combination of the promoter of the gene encoding a protein as shown in the respective line in Table II, application no. 5, column 5 or 8, preferably the coding region thereof, or fragments or homologs thereof with the corresponding encoding gene - becomes a transgenic expression cassette when it is modified by non-natural, synthetic “artificial” methods such as, for example, mutagenesis. Such methods have been described (U.S. Pat. No. 5,565,350; WO 00/15815; also see above).

[0434.1.1.5] to [0435.1.1.5] for the disclosure of these paragraphs see [0434.1.1.1] to [0435.1.1.1] above.

Transgenic plants comprising the respective fine chemical(s) synthesized in the process according to the invention can be marketed directly without isolation of the fine chemical synthesized. In the process according to the invention, plants are understood as meaning all plant parts, plant organs such as leaf, stalk, root, tubers or seeds or propagation material or harvested material or the intact plant. In this context, the seed encompasses all parts of the seed such as the seed coats, epidermal cells, seed cells, endosperm or embryonic tissue. The fine chemical produced in the process according to the invention may, however, also be isolated from the plant. In case of tryptophan this can be in free form or bound to proteins. Fine chemical(s) produced by this process can be harvested by harvesting the non-human organisms either from the culture in which they grow or from the field. For example, this can be done via squeezing, grinding and/or extraction, salt precipitation and/or ion-exchange chromatography of the plant parts, preferably the plant seeds, plant fruits, plant tubers and the like.

[0437.1.1.5] to [0440.1.1.5] for the disclosure of these paragraphs see [0437.1.1.1] to [0440.1.1.1] above.

[0442.1.1.5] to [0454.1.1.5] for the disclosure of these paragraphs see [0442.1.1.1] to [0454.1.1.1] above.

In one embodiment, the present invention relates to a method for the identification of a gene product conferring the production of or an increase in the fine chemical production in a non-human cell, comprising the following steps:
(a) contacting, e.g. hybridizing, a, some or all nucleic acid molecules of a sample, e.g. cells, tissues, plants or microorganisms or a nucleic acid library, which can contain a candidate gene encoding a gene product conferring the production of or an increase in the fine chemical after expression, with the nucleic acid molecule of the present invention as shown in the respective line in column 5 or 8 of Table I A or B of application no. 5, preferably the coding region thereof, or a fragment or homolog thereof;
(b) identifying the nucleic acid molecules, which hybridize under relaxed stringent conditions with the nucleic acid molecule of the present invention in particular to the nucleic acid molecule sequence shown in the respective line in Table I, application no. 5, columns 5 or 8, preferably the coding region thereof, of a fragment or a homolog thereof, preferably in Table I B, application no. 5, columns 5 or 8, preferably the coding region thereof, of a fragment or a homolog thereof, and, optionally, isolating the full length cDNA clone or complete genomic clone;
(c) introducing the candidate nucleic acid molecules or a fragment thereof in host cells, preferably in a plant cell or a microorganism, appropriate for producing the fine chemical;
(d) expressing the identified nucleic acid molecules in the host cells for which the production of or the increased production of the fine chemical is desired;
(e) assaying the fine chemical level in the non-human host cells; and
(f) identifying the nucleic acid molecule and/or its gene product which expression confers the production of or an increase in the fine chemical level in the non-human host cell after expression compared to the wild type.

for the disclosure of this paragraph see [0456.1.1.1] above.

In another embodiment, the present invention relates to a method for the identification of a gene product conferring the production of or an increase in the fine chemical production in a cell, comprising the following steps:
(a) identifying a nucleic acid molecule of a non-human organism, which is at least 20%, 25%, 30%, 35%, 40%, 50%, 60%, 70%, 80%, 90%, 95%, 96%, 97%, 98%, 99%, 99.5% or more homolog to the nucleic acid molecule encoding a polypeptide comprising the polypeptide molecule as shown in the respective line in column 5 or 8 of Table II, application no. 5, or comprising a consensus sequence or a polypeptide motif as shown in the respective line in column 8 of Table IV, application no. 5, or being encoded by a nucleic acid molecule comprising a polynucleotide as shown in the respective line in column 5 or 8 of Table I, application no. 5, preferably the coding region thereof, or a homologue thereof as described herein, for example via homology search in a data bank;
(b) introducing the candidate nucleic acid molecule in non-human host cells, preferably in a plant cell or a microorganism, appropriate for producing the fine chemical;
(c) expressing the identified nucleic acid molecule in the non-human host cells;
(d) assaying the fine chemical level in the non-human host cells; and
(e) identifying the nucleic acid molecule and its gene product which expression confers the production of or an increase in the fine chemical level in the non-human host cell after expression compared to the wild type.

for the disclosure of this paragraph see [0458.1.1.1] above.

Further, the nucleic acid molecules disclosed herein, in particular the nucleic acid molecules shown in the respective line in column 5 or 8 of Table I A or B of application no. 5, may be sufficiently homologous to the sequences of related species such that these nucleic acid molecules may serve as markers for the construction of a genomic map in related organism or for association mapping. Furthermore natural variation in the genomic regions corresponding to nucleic acids disclosed herein, in particular the nucleic acid molecule shown in the respective line in column 5 or 8 of Table I A or B, of application no. 5 or homologous thereof, may lead to variation in the activity of the proteins disclosed herein, in particular the proteins comprising polypeptides as shown in the respective lines in column 5 or 8 of Table II A or B, of application no. 5 or their homologous, or comprising the consensus sequence or the polypeptide motif as shown in the respective line in column 8 of Table IV, and in consequence in a natural variation of an increased fine chemical production.

In consequence natural variation eventually also exists in form of more active allelic variants leading already to a relative increase in the fine chemical. Different variants of the nucleic acid molecules disclosed herein, in particular the nucleic acid molecules comprising the nucleic acid molecules as shown in the respective line in column 5 or 8 of Table I A or B of application no. 5, preferably the coding region thereof, which corresponds to different levels of increase in fine chemical, e.g. different levels of increase in fine chemical, can be indentified and used for marker assisted breeding for an enhanced production of the fine chemical.

Natural variation may also exist in the regulatory regions, e.g. the promoters of the natural genes comprising the nucleic acid molecules as shown in the respective line column 5 or 8 of Table I A or B of application no. 5, preferably the coding region thereof, which can similarly correspond to different levels of increase in the fine chemical, e.g. different levels of increase in the fine chemical can be identified and used for marker assisted breeding for an enhanced production of the fine chemical.

Accordingly, the present invention relates to a method for breeding plants for the production of the fine chemical, comprising
(a) selecting a first plant variety with an increased production of the fine chemical, e.g. based on an increased expression of a nucleic acid molecule of the invention as disclosed herein, in particular of a nucleic acid molecule comprising a nucleic acid molecule as shown in the respective line in column 5 or 8 of Table I A or B of application no. 5, preferably the coding region thereof, or fragments or homologs thereof, or a polypeptide comprising a polypeptide as shown in the respective line in column 5 or 8 of Table II A or B of application no. 5, or fragments or homologs thereof, or comprising a consensus sequence or a polypeptide motif as shown in the respective line in column 8 of Table IV, or a homolog thereof, as described herein;
(b) associating the level of increased production of the fine chemical, with the expression level or the genomic structure of a gene encoding said polypeptide or said nucleic acid molecule;
(c) crossing the first plant variety with a second plant variety, which significantly differs in its level of fine chemical; and
(d) identifying, which of the offspring varieties has got increased production of the fine chemical by the expression level of said polypeptide or nucleic acid molecule or the genomic structure of the gene(s) encoding said polypeptide or nucleic acid molecule of the invention.

[0462.1.1.5] to [0494.1.1.5] for the disclosure of these paragraphs see [0462.1.1.1] to [0494.1.1.1] above.

In a further embodiment the present invention is characterized by the features as disclosed in the following items:

Item 1. A process for the production of a fine chemical selected from the group consisting of:
tryptophan
(a) increasing or generating one or more activities selected from the group consisting of 3-deoxy-7-phosphoheptulonate synthase, 3-ketoacyl-CoA thiolase, acetyltransferase, aldehyde dehydrogenase, amino acid transporter, aromatic amino acid aminotransferase, At1g19800-protein, At2g33510-protein, At4g32480-protein, At5g16650-protein, at5g21910-protein, ATP synthase subunit E, ATP-binding component of a transport system, auxin response factor, b1163-protein, b1346-protein, b1522-protein, b1796-protein, b2739-protein, b2849-protein, b3989-protein, b4029-protein, b4121-protein, binding-protein-dependent transport systems inner membrane component, branched-chain amino acid permease, calcium-dependent protein kinase, carbon dioxide concentrating mechanism protein, cardiolipin synthase, CBL-interacting protein kinase, chaperone protein CIpB, choline kinase, coproporphyrinogen Ill oxidase, cullin, cyclin, cyclin D , cystathionine gamma-synthase, DNA mismatch repair protein, electron transfer flavoprotein subunit beta, elongation factor Tu, enoyl-CoA hydratase, eukaryotic translation initiation factor, eukaryotic translation initiation factor 5, fimbrial-like protein, flavodoxin, geranylgeranyl pyrophosphate synthase, gibberellin 20-oxidase, glucose dehydrogenase, glucose-6-phosphate 1-dehydrogenase, glutamate-ammonia-ligase, glutamine synthetase, glutaredoxin, glycerol kinase, glycosyl transferase, harpin-induced family protein, histone acetyltransferase, hydrolase, imidazole glycerol phosphate synthase subunit, isochorismate synthase, isopentenyl diphosphate isomerase, leucyl/phenylalanyl-tRNA-protein transferase, lipase, L-ribulose-5-phosphate 4-epimerase, malate dehydrogenase, malic enzyme, methyl-accepting chemotaxis protein, methyltransferase, monthiol glutaredoxin, NAD(P)H-quinone oxidoreductase subunit, nitrate/nitrite transport protein, photosystem II protein, polygalacturonase, precorrin methylase, protein kinase, protein phosphatase, putative transport system permease protein, pyruvate kinase, recombinase A, RNA binding protein, RNA-binding protein, Sec-independent protein translocase subunit, short-chain alcohol dehydrogenase family, slr1107-protein, threonine synthase, transaldolase, transcription factor, transcriptional regulator, transport protein, trehalose-phosphatase, tryptophan biosynthesis protein, yfl054c-protein, and Zm_{—}4842_BE510522-protein, in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of tryptophan or a composition comprising tryptophan in said non-human organism or in the culture medium surrounding said non-human organism.

Item 2. A process for the production of a respective fine chemical, which comprises
(A)
(i) increasing or generating of the expression of; and/or
(ii) increasing or generating the expression of an expression product of; and/or
(iii) increasing or generating one or more activities of an expression product encoded by; at least one respective nucleic acid molecule comprising a nucleic acid molecule selected from the group consisting of:
(a) a nucleic acid molecule encoding the polypeptide shown in column 5 or 8 of Table II, preferably Table II B, application no.5, or a homolog or a fragment thereof;
(b) a nucleic acid molecule shown in column 5 or 8 of Table I, preferably Table I B, application no.5, or a homolog or a fragment thereof (preferably the coding region thereof);
(c) a nucleic acid molecule, which, as a result of the degeneracy of the genetic code, can be derived from a polypeptide sequence depicted in column 5 or 8 of Table II, preferably Table II B, application no.5;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 5, or the coding region thereof;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no.5;
(i) a nucleic acid molecule encoding a polypeptide having the activity represented by a respective protein as depicted in column 5 of Table II, application no. 5;
(j) a nucleic acid molecule which comprises a polynucleotide, which is obtained by amplifying a cDNA library or a genomic library using the primers as depicted in column 8 of Table III, application no. 5; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto;
in a non-human organism or a part thereof, preferably a microorganism, a plant cell, a plant or a part thereof, as compared to a corresponding non-transformed wild type non-human organism or a part thereof; and
(B) growing the non-human organism or a part thereof under conditions which permit the production of tryptophan or a composition comprising tryptophan in said non-human organism or in the culture medium surrounding said non-human organism.

Item 3. A process of items 1 or 2, comprising of recovering tryptophan in its free or bound form.

Item 4. The process of any one of items 1 to 3, comprising the following steps:
(a) selecting a non-human organism or a part thereof expressing a polypeptide encoded by the nucleic acid molecule characterized in item 2;
(b) mutagenizing the selected non-human organism or the part thereof;
(c) comparing the activity or the expression level of said polypeptide in the mutagenized non-human organism or the part thereof with the activity or the expression of said polypeptide of the selected non-human organisms or the part thereof;
(d) selecting the mutated non-human organisms or parts thereof, which comprise an increased activity or expression level of said polypeptide compared to the selected non-human organism or the part thereof;
(e) optionally, growing and cultivating the non-human organisms or the parts thereof;
and
(f) recovering, and optionally isolating, the free or bound tryptophan produced by the selected mutated non-human organisms or parts thereof.

Item 5. The process of any one of items 1 to 4 wherein the activity of said protein or the expression of said nucleic acid molecule is increased or generated transiently or stably.

Item 6. An isolated nucleic acid molecule comprising a nucleic acid molecule selected from the group consisting of:
(a) a nucleic acid molecule encoding a polypeptide shown in Table II, application no. 5, column 5 or 8, preferably shown in Table II B, application no. 5, column 8, or a homolog or a fragment thereof;
(b) a nucleic acid molecule shown in Table I, application no. 5, column 5 or 8, preferably shown in Table I B, application no. 5, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 5, preferably in column 8 of Table II B, application no. application no.;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 3, preferably shown in column 8 of Table I B, application no. 5, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 5;
(i) a nucleic acid molecule encoding a polypeptide having the activity represented by a respective protein as depicted in column 5 of Table II, application no. 5;
(j) a nucleic acid molecule comprising a polynucleotide which is obtained by amplifying a cDNA library or a genomic library using the primers in Table III, application no. 5, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (i) ,
or a nucleic acid molecule comprising a sequence which is complementary thereto.

Item 7. A nucleic acid construct which confers the expression of the nucleic acid molecule as defined in item 6, comprising one or more regulatory elements.

Item 8. An expression cassette comprising
(a) a promoter, preferably selected from the group consisting of Big35S, PCUbi, Super and USP;
(b) a nucleic acid molecule as defined in item 6.

Item 9. A vector comprising the nucleic acid molecule as defined in item 6 or the nucleic acid construct as claimed in item 7 or the expression cassette as claimed in item 8.

Item 10. A host cell or a plant organelle, which has been transformed with the vector as claimed in item 9 or the nucleic acid molecule as defined in item 6 or the nucleic acid construct as claimed in item 7 or the expression cassette as claimed in item 8.

Item 11. The host cell of item 10, which is a plant cell or a microorganism.

Item 12. A process for producing a polypeptide, wherein the polypeptide is expressed in a host cell as claimed in item 10 or 11.

Item 13. A polypeptide produced by the process as claimed in item 12 or encoded by the nucleic acid molecule as defined in item 6.

Item 14. An antibody, which binds specifically to the polypeptide as claimed in item 13.

Item 15. A plant tissue, propagation material, harvested material or a plant comprising the host cell as claimed in item 10 or 11.

Item 16. A process for the identification of a compound conferring an increase in tryptophan production in a non-human organism, comprising the steps:
(a) culturing a plant cell or tissue or microorganism or maintaining a plant expressing the polypeptide encoded by the nucleic acid molecule as defined in item 6 conferring an increase in the amount of tryptophan in a non-human organism or a part thereof and a readout system capable of interacting with the polypeptide under suitable conditions which permit the interaction of the polypeptide with said readout system in the presence of a compound or a sample comprising a plurality of compounds and capable of providing a detectable signal in response to the binding of a compound to said polypeptide under conditions which permit the expression of said readout system and of the polypeptide encoded by the nucleic acid molecule of item 6 conferring an increase in the amount of tryptophanin a non-human organism or a part thereof;
(b) identifying if the compound is an effective agonist by detecting the presence or absence or increase of a signal produced by said readout system.

Item 17. A method for the production of an agricultural composition comprising the steps of the method of item 16 and formulating the compound identified in item 16 in a form acceptable for an application in agriculture.

Item 18. A composition comprising the nucleic acid molecule as defined in item 6, the polypeptide as claimed in item 13, the nucleic acid construct as claimed in item 7, the expression cassette as claimed in item 8, the vector as claimed in item 9, the compound as claimed in item 16, the antibody as claimed in item 14, and optionally an agricultural acceptable carrier.

Item 19. Use of the nucleic acid molecule as defined in item 6 for the identification of a nucleic acid molecule conferring an increase in tryptophan after expression.

Item 20. Cosmetic, pharmaceutical, food or feed composition comprising the nucleic acid moleculeas defined in item 6, the polypeptide as claimed in item 13, the nucleic acid construct as claimed in item 7, the expression cassette as claimed in item 8, the vector as claimed in item 9, the antibody as claimed in item 14 , the plant or plant tissue as claimed in item 15, the harvested material as claimed in item 15 or the host cell of item as claimed in items 10 or 11.

Item 21. Use of the nuclei acid molecule as defined in item 6, the polypeptide as claimed in item 13, the nucleic acid construct as claimed in item 7, the expression cassette as claimed in item 8, the vector as claimed in claim9, the plant or plant tissue as claimed in item 15, or the host cell of item 10 to 11 for the production of plant resistant to a herbicide inhibiting the production of tryptophan.

[0483.1.1.5] to [0494.1.1.5] for the disclosure of these paragraphs see [0483.1.1.1] to [0494.1.1.1] above.

### Example 11a

### PCR Amplification of the Sequences

Unless otherwise specified, standard methods as described in Sambrook et al., Molecular Cloning: A laboratory manual, Cold Spring Harbor 1989, Cold Spring Harbor Laboratory Press are used.

The inventive sequences as shown in the respective line in Table I, column 5, preferably the coding region thereof, were amplified by PCR as described in the protocol of the Pfu Ultra, Pfu Turbo or Herculase DNA polymerase (Stratagene). The composition for the protocol of the Pfu Ultra, Pfu Turbo or Herculase DNA polymerase was as follows: 1×PCR buffer (Stratagene), 0.2 mM of each dNTP, 100 ng genomic DNA of Saccharomyces cerevisiae (strain S288C; Research Genetics, Inc., now Invitrogen), Escherichia coli (strain MG1655; E. coli Genetic Stock Center), Synechocystis sp. (strain PCC6803), Azotobacter vinelandii (strain N.R. Smith,16), Thermus thermophilus (HB8) or 50 ng cDNA from various tissues and development stages of Arabidopsis thaliana (ecotype Columbia), Physcomitrella patens, Glycine max (variety Resnick), Brassica napus, Oryza sativa or Zea mays (variety B73, Mo17, A188), 50 pmol forward primer, 50 pmol reverse primer, with or without 1 M Betaine, 2.5 u Pfu Ultra, Pfu Turbo or Herculase DNA polymerase.

The amplification cycles were as follows:

1 cycle of 2-3 minutes at 94-95° C., then 25-36 cycles with 30-60 seconds at 94-95° C., 30-45 seconds at 50-60° C. and 210-480 seconds at 72° C., followed by 1 cycle of 5-10 minutes at 72° C., then 4-16° C.—preferably for Saccharomyces cerevisiae, Escherichia coli, Synechocystis sp., Azotobacter vinelandii, Thermus thermophilus.

In case of Arabidopsis thaliana, Brassica napus, Glycine max, Oryza sativa, Physcomitrella patens, Zea mays the amplification cycles were as follows:

1 cycle with 30 seconds at 94° C., 30 seconds at 61° C., 15 minutes at 72° C., then 2 cycles with 30 seconds at 94° C., 30 seconds at 60° C., 15 minutes at 72° C., then 3 cycles with 30 seconds at 94° C., 30 seconds at 59° C., 15 minutes at 72° C., then 4 cycles with 30 seconds at 94° C., 30 seconds at 58° C., 15 minutes at 72° C., then 25 cycles with 30 seconds at 94° C., 30 seconds at 57° C., 15 minutes at 72° C., then 1 cycle with 10 minutes at 72° C., then finally 4-16° C.

RNAs were generated with the RNeasy Plant Kit according to the standard protocol (Qiagen) and Superscript II Reverse Transkriptase was used to produce double stranded cDNA according to the standard protocol (Invitrogen).

ORF specific primer pairs for the genes to be expressed are shown in the respective line in Table III, column 8. The following adapter sequences were added to Saccharomyces cerevisiae ORF specific primers (see Table III) for cloning purposes:

SEQ ID NO: 20
i) foward primer: 5′-GGAATTCCAGCTGACCACC-3′
SEQ ID NO: 21
ii) reverse primer: 5′-GATCCCCGGGAATTGCCATG-3′

These adaptor sequences allow cloning of the ORF into the various vectors containing the Resgen adaptors, see table column E of Table c.

The following adapter sequences were added to Saccharomyces cerevisiae, Escherichia coli, Synechocystis sp., Azotobacter vinelandii, Thermus thermophilus, Arabidopsis thaliana, Brassica napus, Glycine max, Oryza sativa , Physcomitrella patens, or Zea mays ORF specific primers for cloning purposes:

SEQ ID NO: 22
iii) forward primer: 5′-TTGCTCTTCC-3′
SEQ ID NO: 23
iiii) reverse primer: 5′-TTGCTCTTCG-3′

The adaptor sequences allow cloning of the ORF into the various vectors containing the Colic adaptors, see table column E of Table c.

Therefore for amplification and cloning of Saccharomyces cerevisiae SEQ ID NO: 14843, a primer consisting of the adaptor sequence i) and the ORF specific sequence SEQ ID NO: 14871 and a second primer consisting of the adaptor sequence ii) and the ORF specific sequence SEQ ID NO: 14872 were used.

For amplification and cloning of Escherichia coli SEQ ID NO: 7333, a primer consisting of the adaptor sequence iii) and the ORF specific sequence SEQ ID NO: 7679 and a second primer consisting of the adaptor sequence iiii) and the ORF specific sequence SEQ ID NO: 7680 were used.

For amplification and cloning of Synechocystis sp. SEQ ID NO: 11471, a primer consisting of the adaptor sequence iii) and the ORF specific sequence SEQ ID NO: 11983 and a second primer consisting of the adaptor sequence iiii) and the ORF specific sequence SEQ ID NO: 11984 were used.

For amplification and cloning of Arabidopsis thaliana SEQ ID NO: 1815, a primer consisting of the adaptor sequence iii) and the ORF specific sequence SEQ ID NO: 2361 and a second primer consisting of the adaptor sequence iiii) and the ORF specific sequence SEQ ID NO: 2362 were used.

For amplification and cloning of Azotobacter vinelandii SEQ ID NO: 5557, a primer consisting of the adaptor sequence iii) and the ORF specific sequence SEQ ID NO: 6033 and a second primer consisting of the adaptor sequence iiii) and the ORF specific sequence SEQ ID NO: 6034 were used.

For amplification and cloning of Glycine max SEQ ID NO: 16883, a primer consisting of the adaptor sequence iii) and the ORF specific sequence SEQ ID NO: 17345 and a second primer consisting of the adaptor sequence iiii) and the ORF specific sequence SEQ ID NO: 17346 were used.

For amplification and cloning of Physcomytrella patens SEQ ID NO: 101780, a primer consisting of the adaptor sequence iii) and the ORF specific sequence SEQ ID NO: 101852 and a second primer consisting of the adaptor sequence iiii) and the ORF specific sequence SEQ ID NO: 101853 were used.

For amplification and cloning of Thermus thermophilus SEQ ID NO: 12698, a primer consisting of the adaptor sequence iii) and the ORF specific sequence SEQ ID NO: 12970 and a second primer consisting of the adaptor sequence iiii) and the ORF specific sequence SEQ ID NO: 12971 were used.

For amplification and cloning of Zea mays SEQ ID NO: 68413, a primer consisting of the adaptor sequence iii) and the ORF specific sequence SEQ ID NO: 68531 and a second primer consisting of the adaptor sequence iiii) and the ORF specific sequence SEQ ID NO: 68532 were used.

Following these examples every sequence disclosed in Table I, preferably column 5, especially the coding region thereof can be cloned by fusing the adaptor sequences to the respective specific primers sequences as disclosed in Table III, column 8 using the respective vectors shown in Table c.

[0496.1.1.5] to [0499.1.1.5] for the disclosure of these paragraphs see [0496.1.1.1] to [0499.1.1.1] above.

### Example 11e

### Cloning of Inventive Sequences as Shown in Table I, Column 5 in the Different Expression Vectors

For cloning of for example SEQ ID NO: 7333 from Escherichia coli or any other ORF from Escherichia coli, Synechocystis sp., Azotobacter vinelandii, Thermus thermophilus, Arabidopsis thaliana, Glycine max, Physcomitrella patens, or Zea mays the vector DNA was treated with the restriction enzymes Pacl and Ncol following the standard protocol (MBI Fermentas).

For cloning for example the ORFs of SEQ ID NO: 14843 from Saccharomyces cerevisiae or any other ORF from Saccharomyces cerevisiae into vectors containing the Resgen adaptor sequence the respective vector DNA was treated with the restriction enzyme Ncol.

For cloning of for example ORFs SEQ ID NO: 66274 from Saccharomyces cerevisiae or any other ORF from Saccharomyces cerevisiae into vectors containing the Colic adaptor sequence, the respective vector DNA was treated with the restriction enzymes Pacl and Ncol following the standard protocol (MBI Fermentas).

In all cases the reaction was stopped by inactivation at 70° C. for 20 minutes and purified over QIAquick or NucleoSpin Extract II columns following the standard protocol (Qiagen or Macherey-Nagel).

Then the PCR-product representing the amplified ORF with the respective adapter sequences and the vector DNA were treated with T4 DNA polymerase according to the standard protocol (MBI Fermentas) to produce single stranded overhangs with the parameters 1 unit T4 DNA polymerase at 37° C. for 2-10 minutes for the vector and 1-2 u T4 DNA polymerase at 15-17° C. for 10-60 minutes for the PCR product representing SEQ ID NO: 7333.

The reaction was stopped by addition of high-salt buffer and purified over QIAquick or NucleoSpin Extract II columns following the standard protocol (Qiagen or Macherey-Nagel).

According to this example the skilled person is able to clone all sequences disclosed in Table I, preferably column 5 or column 8, especially the coding region thereof.

Approximately 30-60 ng of prepared vector and a defined amount of prepared amplificate were mixed and hybridized at 65° C. for 15 minutes followed by 37° C. 0,1 ° C./1 seconds, followed by 37° C. 10 minutes, followed by 0.1° C./1 seconds, then 4-10 ° C.

The ligated constructs were transformed in the same reaction vessel by addition of competent E. coli cells (strain DHSalpha) and incubation for 20 minutes at 1 ° C. followed by a heat shock for 90 seconds at 42° C. and cooling to 1-4° C. Then, complete medium (SOC) was added and the mixture was incubated for 45 minutes at 37° C. The entire mixture was subsequently plated onto an agar plate with 0.05 mg/ml kanamycin and incubated overnight at 37° C.

The outcome of the cloning step was verified by amplification with the aid of primers which bind upstream and downstream of the integration site, thus allowing the amplification of the insertion. The amplifications were carried out as described in the protocol of Taq DNA polymerase (Gibco-BRL).

The amplification cycles were as follows: 1 cycle of 1-5 minutes at 94° C., followed by 35 cycles of in each case 15-60 seconds at 94° C., 15-60 seconds at 50-66° C. and 5-15 minutes at 72° C., followed by 1 cycle of 10 minutes at 72° C., then 4-16° C.

Several colonies were checked, but only one colony for which a PCR product of the expected size was detected was used in the following steps.

A portion of this positive colony was transferred into a reaction vessel filled with complete medium (LB) supplemented with kanamycin and incubated overnight at 37° C.

The plasmid preparation was carried out as specified in the Qiaprep or NucleoSpin Multi-96 Plus standard protocol (Qiagen or Macherey-Nagel).

[0501.1.1.5] to [0503.1.1.5] for the disclosure of these paragraphs see [0501.1.1.1] to [0503.1.1.1] above. Table d showing results of plant analyses

TABLE d

| SeqID | Target | Locus | Metabolite | Source | Promotor | Method | Min | Max |
|---|---|---|---|---|---|---|---|---|
| 16883 | plastidic | 59582753_ | tryptophan | ARA_LEAF | p-PcUBI | LC | 28 | 92 |
| | | SOYBEAN | | | | | | |
| 17637 | non- | At1g19800 | tryptophan | ARA_LEAF | p-PcUBI | LC | 30 | 443 |
| | targeted | | | | | | | |
| 17701 | non- | At1g26830 | tryptophan | ARA_LEAF | p-PcUBI | LC | 32 | 117 |
| | targeted | | | | | | | |
| 17968 | non- | At1g36730 | tryptophan | ARA_LEAF | p-PcUBI | LC | 39 | 78 |
| | targeted | | | | | | | |
| 18235 | non- | At1g48260 | tryptophan | ARA_LEAF | p-PcUBI | LC | 36 | 239 |
| | targeted | | | | | | | |
| 19502 | non- | At2g17560 | tryptophan | ARA_LEAF | p-PcUBI | LC | 34 | 179 |
| | targeted | | | | | | | |
| 19874 | non- | At2g28890 | tryptophan | ARA_LEAF | p-PcUBI | LC | 32 | 330 |
| | targeted | | | | | | | |
| 19919 | non- | At2g30360 | tryptophan | ARA_LEAF | p-PcUBI | LC | 46 | 700 |
| | targeted | | | | | | | |
| 20346 | non- | At2g30540 | tryptophan | ARA_LEAF | p-PcUBI | LC | 28 | 126 |
| | targeted | | | | | | | |
| 98522 | non- | At2g33510 | tryptophan | ARA_LEAF | p-PcUBI | LC | 34 | 59 |
| | targeted | | | | | | | |
| 21159 | non- | At2g47880 | tryptophan | ARA_LEAF | p-PcUBI | LC | 33 | 380 |
| | targeted | | | | | | | |
| 21497 | plastidic | At3g04050 | tryptophan | ARA_LEAF | p-PcUBI | LC | 76 | 131 |
| 22611 | non- | At3g11650 | tryptophan | ARA_LEAF | p-PcUBI | LC | 29 | 146 |
| | targeted | | | | | | | |
| 22832 | non- | At3g18524 | tryptophan | ARA_LEAF | p-PcUBI | LC | 41 | 74 |
| | targeted | | | | | | | |
| 1815 | non- | At3g23000 | tryptophan | ARA_LEAF | p-PcUBI | LC | 105 | 239 |
| | targeted | | | | | | | |
| 68727 | non- | At3g26400 | tryptophan | ARA_LEAF | p-PcUBI | LC | 30 | 77 |
| | targeted | | | | | | | |
| 68777 | non- | At3g27540 | tryptophan | ARA_LEAF | p-PcUBI | LC | 35 | 148 |
| | targeted | | | | | | | |
| 2367 | non- | At3g61830 | tryptophan | ARA_LEAF | p-PcUBI | LC | 45 | 123 |
| | targeted | | | | | | | |
| 2573 | non- | At3g62930 | tryptophan | ARA_LEAF | p-PcUBI | LC | 38 | 123 |
| | targeted | | | | | | | |
| 2935 | non- | At3g62950 | tryptophan | ARA_LEAF | p-PcUBI | LC | 29 | 86 |
| | targeted | | | | | | | |
| 4040 | non- | At4g32480 | tryptophan | ARA_LEAF | p-PcUBI | LC | 41 | 460 |
| | targeted | | | | | | | |
| 24311 | non- | At4g34160 | tryptophan | ARA_LEAF | p-PcUBI | LC | 36 | 123 |
| | targeted | | | | | | | |
| 4348 | non- | At4g35310 | tryptophan | ARA_LEAF | p-PcUBI | LC | 62 | 231 |
| | targeted | | | | | | | |
| 68987 | non- | At5g02760 | tryptophan | ARA_LEAF | p-PcUBI | LC | 30 | 228 |
| | targeted | | | | | | | |
| 24492 | non- | At5g07200 | tryptophan | ARA_LEAF | p-PcUBI | LC | 33 | 57 |
| | targeted | | | | | | | |
| 25283 | non- | At5g16650 | tryptophan | ARA_LEAF | p-PcUBI | LC | 32 | 83 |
| | targeted | | | | | | | |
| 69553 | non- | At5g21910 | tryptophan | ARA_LEAF | p-PcUBI | LC | 36 | 57 |
| | targeted | | | | | | | |
| 25428 | non- | At5g39760 | tryptophan | ARA_LEAF | p-PcUBI | LC | 28 | 121 |
| | targeted | | | | | | | |
| 70006 | non- | At5g60110 | tryptophan | ARA_LEAF | p-PcUBI | LC | 43 | 60 |
| | targeted | | | | | | | |
| 5557 | non- | Avin- | tryptophan | ARA_LEAF | p-PcUBI | LC | 62 | 203 |
| | targeted | DRAFT_1495 | | | | | | |
| 27021 | non- | Avin- | tryptophan | ARA_LEAF | p-PcUBI | LC | 34 | 173 |
| | targeted | DRAFT_2344 | | | | | | |
| 28738 | non- | Avin- | tryptophan | ARA_LEAF | p-PcUBI | LC | 46 | 139 |
| | targeted | DRAFT_3159 | | | | | | |
| 29286 | non- | Avin- | tryptophan | ARA_LEAF | p-PcUBI | LC | 34 | 143 |
| | targeted | DRAFT_3209 | | | | | | |
| 73038 | non- | Avin- | tryptophan | ARA_LEAF | p-PcUBI | LC | 29 | 81 |
| | targeted | DRAFT_4420 | | | | | | |
| 74163 | non- | Avin- | tryptophan | ARA_LEAF | p-PcUBI | LC | 35 | 163 |
| | targeted | DRAFT_4836 | | | | | | |
| 6510 | non- | Avin- | tryptophan | ARA_LEAF | p-PcUBI | LC | 41 | 75 |
| | targeted | DRAFT_5103 | | | | | | |
| 33596 | non- | Avin- | tryptophan | ARA_LEAF | p-PcUBI | LC | 59 | 124 |
| | targeted | DRAFT_6093 | | | | | | |
| 74729 | non- | Avin- | tryptophan | ARA_LEAF | p-PcUBI | LC | 39 | 126 |
| | targeted | DRAFT_6679 | | | | | | |
| 34889 | plastidic | B0004 | tryptophan | ARA_LEAF | p-Super | LC | 57 | 168 |
| 98553 | non- | B0008 | tryptophan | ARA_SEED_2 | p-USP | GC | 51 | 91 |
| | targeted | | | | | | | |
| 35204 | non- | B0061 | tryptophan | ARA_LEAF | p-USP | LC | 72 | 173 |
| | targeted | | | SEED_2 | | | | |
| 35482 | non- | B0124 | tryptophan | ARA_LEAF | p-Super | LC | 29 | 213 |
| | targeted | | | | | | | |
| 7333 | non- | B0449 | tryptophan | ARA_LEAF | p-Super | LC | 38 | 281 |
| | targeted | | | | | | | |
| 35967 | plastidic | B0593 | tryptophan | ARA_LEAF | p-Super | LC | 43 | 358 |
| 36489 | non- | B0885 | tryptophan | ARA_LEAF | p-Super | LC | 39 | 143 |
| | targeted | | | | | | | |
| 37394 | non- | B1163 | tryptophan | ARA_LEAF | p-Super | LC | 30 | 56 |
| | targeted | | | | | | | |
| 98778 | plastidic | B1249 | tryptophan | ARA_SEED_2 | p-USP | GC | 71 | 214 |
| 76157 | plastidic | B1262 | tryptophan | ARA_SEED_2 | p-USP | GC | 74 | 104 |
| 37807 | non- | B1300 | tryptophan | ARA_LEAF | p-Super | LC | 31 | 68 |
| | targeted | | | | | | | |
| 99188 | plastidic | B1346 | tryptophan | ARA_LEAF | p-Super | LC | 31 | 96 |
| 7947 | non- | B1522 | tryptophan | ARA_LEAF | p-Super | LC | 109 | 261 |
| | targeted | | | | | | | |
| 99196 | non- | B1796 | tryptophan | ARA_LEAF | p-Super | LC | 27 | 39 |
| | targeted | | | | | | | |
| 99210 | non- | B1886 | tryptophan | ARA_SEED_2 | p-USP | LC | 54 | 108 |
| | targeted | | | | | | | |
| 79056 | non- | B1897 | tryptophan | ARA_LEAF | p-Super | LC | 44 | 190 |
| | targeted | | | | | | | |
| 99371 | non- | B2023 | tryptophan | ARA_SEED_2 | p-USP | LC | 44 | 148 |
| | targeted | | | | | | | |
| 38947 | non- | B2063 | tryptophan | ARA_LEAF | p-Super | LC | 35 | 270 |
| | targeted | | | | | | | |
| 99620 | non- | B2285 | tryptophan | ARA_SEED_2 | p-USP | GC | 57 | 109 |
| | targeted | | | | | | | |
| 99898 | non- | B2546 | tryptophan | ARA_LEAF | p-Super | LC | 32 | 95 |
| | targeted | | | | | | | |
| 100105 | non- | B2699 | tryptophan | ARA_SEED_2 | p-USP | LC | 55 | 82 |
| | targeted | | | | | | | |
| 80906 | non- | B2739 | tryptophan | ARA_LEAF | p-Super | LC | 75 | 134 |
| | targeted | | | | | | | |
| 81980 | non- | B2849 | tryptophan | ARA_LEAF | p-Super | LC | 33 | 152 |
| | targeted | | | | | | | |
| 100543 | non- | B3142 | tryptophan | ARA_LEAF | p-Super | LC | 35 | 65 |
| | targeted | | | | | | | |
| 42502 | non- | B3262 | tryptophan | ARA_LEAF | p-Super | LC | 47 | 277 |
| | targeted | | | | | | | |
| 100675 | plastidic | B3845 | tryptophan | ARA_SEED_2 | p-USP | GC | 56 | 87 |
| 101327 | plastidic | B3870 | tryptophan | ARA_SEED_2 | p-USP | LC | 47 | 85 |
| 44372 | non- | B3989 | tryptophan | ARA_LEAF | p-Super | LC | 32 | 398 |
| | targeted | | | | | | | |
| 10740 | non- | B4029 | tryptophan | ARA_LEAF | p-Super | LC | 27 | 814 |
| | targeted | | | | | | | |
| 44609 | non- | B4121 | tryptophan | ARA_LEAF | p-Super | LC | 30 | 46 |
| | targeted | | | | | | | |
| 45321 | non- | B4256 | tryptophan | ARA_LEAF | p-Super | LC | 34 | 217 |
| | targeted | | | | | | | |
| 46515 | non- | GM | tryptophan | ARA_LEAF | p-PcUBI | LC | 37 | 149 |
| | targeted | 02LC17556 | | | | | | |
| 84198 | non- | GM | tryptophan | ARA_LEAF | p-PcUBI | LC | 43 | 224 |
| | targeted | 02LC21368 | | | | | | |
| 101780 | non- | S_pp | tryptophan | ARA_LEAF | p-Super | LC | 40 | 55 |
| | targeted | 020029328r | | | | | | |
| 49143 | plastidic | Sll0248 | tryptophan | ARA_LEAF | p-PcUBI | LC | 32 | 169 |
| 50339 | plastidic | Sll0521 | tryptophan | ARA_LEAF | p-PcUBI | LC | 31 | 49 |
| 101707 | non- | Sll0833 | tryptophan | ARA_LEAF | p-PcUBI | LC | 42 | 92 |
| | targeted | | | | | | | |
| 52634 | mito- | Sll1031 | tryptophan | ARA_LEAF | p-PcUBI | LC | 28 | 167 |
| | chondrial | | | | | | | |
| 54337 | mito- | Sll1450 | tryptophan | ARA_LEAF | p-PcUBI | LC | 42 | 85 |
| | chondrial | | | | | | | |
| 11471 | mito- | Sll1917 | tryptophan | ARA_LEAF | p-PcUBI | LC | 31 | 36 |
| | chondrial | | | | | | | |
| 86540 | mito- | Slr0239 | tryptophan | ARA_LEAF | p-PcUBI | LC | 37 | 162 |
| | chondrial | | | | | | | |
| 87397 | plastidic | Slr0721 | tryptophan | ARA_LEAF | p-PcUBI | LC | 46 | 100 |
| 57235 | plastidic | Slr0739 | tryptophan | ARA_LEAF | p-PcUBI | LC | 35 | 54 |
| 101776 | plastidic | Slr1107 | tryptophan | ARA_LEAF | p-PcUBI | LC | 30 | 54 |
| 58731 | plastidic | Slr1739 | tryptophan | ARA_LEAF | p-PcUBI | LC | 86 | 157 |
| 59370 | plastidic | Slr2124 | tryptophan | ARA_LEAF | p-PcUBI | LC | 38 | 80 |
| 12698 | non- | TTC0019 | tryptophan | ARA_LEAF | p-PcUBI | LC | 306 | 472 |
| | targeted | | | | | | | |
| 88879 | plastidic | Ybr249c | tryptophan | ARA_LEAF | p-Super | LC | 59 | 405 |
| 63544 | plastidic | Ydr046c | tryptophan | ARA_LEAF | p-Super | LC | 30 | 63 |
| 64148 | plastidic | Yfl054c | tryptophan | ARA_LEAF | p-Super | LC | 30 | 41 |
| 101855 | plastidic | Yhl032c | tryptophan | ARA_SEED_2 | p-USP | GC | 61 | 101 |
| 102015 | plastidic | Yhr137w | tryptophan | ARA_LEAF | p-PcUBI | LC | 29 | 77 |
| 14843 | non- | Yjr130c | tryptophan | ARA_LEAF | Big35S | LC | 30 | 62 |
| | targeted | | | | | | | |
| 66274 | non- | Yjr153w | tryptophan | ARA_LEAF | p-PcUBI | LC | 34 | 115 |
| | targeted | | | | | | | |
| 102061 | mito- | Ykr089c | tryptophan | ARA_SEED_2 | p-USP | GC | 60 | 288 |
| | chondrial | | | | | | | |
| 92339 | plastidic | Ynl101w | tryptophan | ARA_LEAF | p-Super | LC | 33 | 45 |
| 92540 | plastidic | Yor244w | tryptophan | ARA_LEAF | p-Super | LC | 39 | 135 |
| 67998 | plastidic | Ypl117c | tryptophan | ARA_LEAF | p-PcUBI | LC | 29 | 59 |
| 98445 | non- | Ypr119w | tryptophan | ARA_LEAF | Big35S | LC | 36 | 54 |
| | targeted | | | | | | | |
| 68413 | non- | Zm_4842_ | tryptophan | ARA_LEAF | p-PcUBI | LC | 53 | 117 |
| | targeted | BE510522 | | | | | | |

Column 1 shows the SEQ ID NO, Column 2 shows the expression type (targeted or non-targeted), Column 3 shows the “gene name” (locus), Column 4 shows the metabolite analyzed, Column 5 indicates the A. thaliana source tissue analyzed, Column 6 indicates the used promoter for expression, Column 7 indicates the analytical method. Columns 8 and 9 show the minumum and the maximum increase of the analyzed metabolite (in percent) in comparison to the wild type (=ratio_by_weight, given as percent increase).

When the analyses were repeated independently, all results proved to be significant.

[0505.1.1.5] to [0515.1.1.5] for the disclosure of these paragraphs see [0505.1.1.1] to [0515.1.1.1] above.

In a further embodiment, the present invention relates in paragraphs [0000.1.1.6.] to [0514.1.1.6.] to a further process for the production of the fine chemical isoleucine, leucine or valine, respectively, as defined below and corresponding embodiments as described herein as follows.

[00011.1.6]0 to [0008.1.1.6] for the disclosure of these paragraphs see [0001.1.1.1] to [0008.1.1.1] above.

As described above, the essential amino acids are necessary for humans and many mammals, for example for livestock. The branched-chain amino acids (BCAA) leucine, isoleucine and valine are among the nine dietary indispensable amino acids for humans. BCAA accounts for 35-40% of the dietary indispensable amino acids in body protein and 14% of the total amino acids in skeletal muscle (Ferrando et al., J. Parenter. Enteral Nutr. 19, 47 (1995)). They share a common membrane transport system and enzymes for their transamination and irreversible oxidation (Block K. P., “Interactions among leucine, isoleucine, and valine with special reference to the branched chain amino acid antagonism” in “Absorption and Utilization of Amino Acids”, Friedman M. (Ed.), pp. 229-244, 1989, CRC Press, Boca Raton, Fla.; Champe P. C., Harvey R. A., “Amino acids: metabolism of carbon atoms” in “Biochemistry”, Champ P. C., Harvery P. A.,(Eds.), pp.242-252, 1987, Lippincott J. B., Philadelphia, Pa.). Further, for patient suffering from Maple Syrup Urine Disease (MSUD) a reduced uptake of those branched-chain amino acids is essential. Dietary sources of the branched-chain amino acids are principally derived from animal and vegetable proteins. The branched-chain amino acids leucine, isoleucine and valine are limiting for the growth of many mammals. Therefore the branched-chain amino acids are supplemented in the feed of broiler, leg hens, turkey, swine or cattle diets.

[0010.1.1.6] to [0011.1.1.6] for the disclosure of these paragraphs see [0010.1.1.1] to [0011.1.1.1] above.

It is an object of the present invention to develop an inexpensive process for the synthesis of isoleucine, leucine, and/or valine.

for the disclosure of this paragraph see [0013.1.1.1] above.

Accordingly, in a first embodiment, the invention relates to a process for the production of at least one fine chemical selected from the group consisting of: isoleucine, leucine and valine, or, in other words, of the “fine chemical” or “fine chemical of the invention”.

Accordingly, in a first embodiment, the invention relates to a process for the production of a fine chemical, whereby the fine chemical is leucine or isoleucine or valine, preferably L-leucine or Lisoleucine or L-valine, as the case may be. Accordingly, in the present invention, the term “the fine chemical” as used herein relates to “leucine or isoleucine or valine” as th ecase may be.

Further, the term “the fine chemicals” as used herein also relates to fine chemicals comprising leucine or isoleucine or valine.

The terms “fine chemical of the invention”, “fine chemical” or “the fine chemical” are used herein equally and relate in context of the paragraphs or sections [0014.1.6.6] to [0514.1.1.6] essentially to the metabolite or the metabolites indicated in column 7, application no. 6 of Tables I, II and IV in the respective line.

Further, the term “in context of any of the paragraphs [0014.1.6.6] to [0514.1.1.6]” as used herein means that for any of said paragraphs [0014.1.6.6] to [0514.1.1.6] the term “the fine chemical” is understood to follow the definition of paragraphs or sections [0014.1.6.6] and [0015.1.6.6], independently whether it refers to any other paragraph or not and whether the reference recites the term “fine chemical” in an other context.

Thus, in cases where one or more paragraphs or sections are incorporated by reference into any of the present paragraphs [0014.1.6.6] to [0514.1.1.6], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to the definition of paragraph [0014.1.6.6] and [0015.1.6.6].

Accordingly, in one embodiment, the term “fine chemical of the invention”, “fine chemical” or “the fine chemical” as used herein (alone or in combination, like in FCRP) means “isoleucine” in context of the nucleic acid or polypeptide sequences listed in the respective same line of any one of Tables I to IV of application no. 6 and indicating in column 7 the metabolite “isoleucine”. In one embodiment, the term isoleucine or the term “fine chemical” mean in context of the paragraphs or sections [0014.1.6.6] to [0514.1.1.6] at least one chemical compound with an activity of the above mentioned isoleucine, respectively.

Accordingly, in one embodiment, the term “fine chemical of the invention”, “fine chemical” or “the fine chemical” as used herein (alone or in combination, like in FCRP) means “leucine” in context of the nucleic acid or polypeptide sequences listed in the respective same line of any one of Tables I to IV of application no. 6 and indicating in column 7 the metabolite “leucine”. In one embodiment, the term leucine or the term “fine chemical” mean in context of the paragraphs or sections [0014.1.6.6] to [0514.1.1.6] at least one chemical compound with an activity of the above mentioned leucine, respectively.

Accordingly, in one embodiment, the term “fine chemical of the invention”, “fine chemical” or “the fine chemical” as used herein (alone or in combination, like in FCRP) means “valine” in context of the nucleic acid or polypeptide sequences listed in the respective same line of any one of Tables I to IV of application no. 6 and indicating in column 7 the metabolite “valine”. In one embodiment, the term valine or the term “fine chemical” mean in context of the paragraphs or sections [0014.1.6.6] to [0514.1.1.6] at least one chemical compound with an activity of the above mentioned valine, respectively.

Accordingly, in one embodiment, the terms “fine chemical of the invention”, “fine chemical” or “the fine chemical” mean in context of any of the paragraphs [0014.1.6.6] to [0514.1.1.6] isoleucine, preferably the L-enantiomer of isoleucine, its salts, ester or amides in free form or boundform. In a preferred embodiment, the term “the fine chemical” means isoleucine or its salts, in free form or boundform. In a preferred embodiment, the term “the fine chemical” means the L-enantiomer of isoleucine.

On the other hand in case “isoleucine” is stated it means isoleucine itself, its salts, ester or amides in free form or bound form, preferably the L-enantiomer of isoleucine, its salts, ester or amides in free form or bound form. In a preferred embodiment “isoleucine” means the Lenantiomer of isoleucine in free form. In another preferred embodiment “isoleucine” means the L-enantiomer of isoleucine bound form.

Accordingly, in one embodiment, the terms “fine chemical of the invention”, “fine chemical” or “the fine chemical” mean in context of any of the paragraphs [0014.1.6.6] to [0514.1.1.6] leucine, preferably the L-enantiomer of leucine, its salts, ester or amides in free form or boundform.

In a preferred embodiment, the term “the fine chemical” means leucine or its salts, in free form or bound form. In a preferred embodiment, the term “the fine chemical” means the L-enantiomer of leucine.

On the other hand in case “leucine” is stated it means leucine itself, its salts, ester or amides in free form or bound form, preferably the L-enantiomer of leucine, its salts, ester or amides in free form or bound form. In a preferred embodiment “leucine” means the L-enantiomer of leucine in free form. In another preferred embodiment “leucine” means the L-enantiomer of leucine bound form.

Accordingly, in one embodiment, the terms “fine chemical of the invention”, “fine chemical” or “the fine chemical” mean in context of any of the paragraphs [0014.1.6.6] to [0514.1.1.6] valine, preferably the L-enantiomer of valine, its salts, ester or amides in free form or bound form. In a preferred embodiment, the term “the fine chemical” means valine or its salts, in free form or boundform. In a preferred embodiment, the term “the fine chemical” means the L-enantiomer of valine.

On the other hand in case “valine” is stated it means valine itself, its salts, ester or amides in free form or bound form, preferably the L-enantiomer of valine, its salts, ester or amides in free form or bound form. In a preferred embodiment “valine” means the L-enantiomer of valine in free form. In another preferred embodiment “valine” means the L-enantiomer of valine bound form.

Further, the term “in context of any of the paragraphs [0014.1.6.6] to [0514.1.1.6]” as used herein means that for any of said paragraphs [0014.1.6.6] to [0514.1.1.6] the term “the fine chemical” is understood to follow the definition of section [0014.1.6.6] or section [0015.1.6.6], independently whether it refers to any other paragraph or not and whether the reference recites the term “fine chemical” in an other context.

Thus, in cases where one or more paragraphs or sections are incorporated by reference into any of the present paragraphs [0014.1.6.6] to [0514.1.1.6], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to this definition of this paragraph [0015.1.6.6].

Further, the term “fine chemicals” as used herein relates to compositions comprising said fine chemical(s), i.e. comprising isoleucine, or comprising leucine, or compring valine, or comprising at least one of isoleucine, leucine and valine, respectively.

Accordingly, the present invention relates to a process for the production of isoleucine, which comprises
(a) increasing or generating one or more activities selected from the group consisting of 47266012-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of glucose-6-phosphate 1-dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of glucose-6-phosphate 1-dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of glucose-6-phosphate 1-dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of DNA-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of At1g19800-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of At1g19800-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of At1g23110-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of cullin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of cullin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of eukaryotic translation initiation factor 5 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of eukaryotic translation initiation factor 5 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating one or more activities selected from the group consisting of At1g47380-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of At1g47380-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of CBL-interacting protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of CBL-interacting protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of CBL-interacting protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of thioredoxin family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of thioredoxin family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of harpin-induced family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of harpin-induced family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of DNA mismatch repair protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of DNA mismatch repair protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of DNA mismatch repair protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of eukaryotic translation initiation factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of eukaryotic translation initiation factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of eukaryotic translation initiation factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of glycosyl transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of glycosyl transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of glycosyl transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of auxin response factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of auxin response factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of auxin response factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of At4g32480-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of At4g32480-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of At4g32480-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating one or more activities selected from the group consisting of At5g16650-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating one or more activities selected from the group consisting of At5g16650-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating one or more activities selected from the group consisting of thioredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of thioredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of disease resistance protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of RNA-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of MADS-box transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of MADS-box transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of zinc finger protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of malic enzyme in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of malic enzyme in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of malic enzyme in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of beta-hydroxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of elongation factor Tu in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of elongation factor Tu in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of elongation factor Tu in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of 50S ribosomal protein L14 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of enoyl-CoA hydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of enoyl-CoA hydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of enoyl-CoA hydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of acyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of acyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of acyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of ABC transporter component in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of glucose-1-phosphate cytidylyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of aminotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating one or more activities selected from the group consisting of aminotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of NADH-quinone oxidoreductase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of NADH-quinone oxidoreductase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of glutamate-ammonia-ligase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of glutamate-ammonia-ligase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of glutamate-ammonia-ligase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of isopropylmalate isomerase large subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of fumarylacetoacetate hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of fumarylacetoacetate hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of fumarylacetoacetate hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating one or more activities selected from the group consisting of peptidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of chaperone protein CIpB in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating one or more activities selected from the group consisting of purine nucleoside phosphorylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of purine nucleoside phosphorylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of purine nucleoside phosphorylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of threonine synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of threonine synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of threonine synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of b0050-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating one or more activities selected from the group consisting of acetolactate synthase small subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of leucine, which comprises
(a) increasing or generating one or more activities selected from the group consisting of betagalactosidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of 2,3-dihydroxyphenylpropionate 1,2-dioxygenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating one or more activities selected from the group consisting of 2,3-dihydroxyphenylpropionate 1,2-dioxygenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of ATP-binding component of a transport system in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of ATP-binding component of a transport system in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of ATP-binding component of a transport system in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of membrane transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating one or more activities selected from the group consisting of isochorismate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of isochorismate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of isochorismate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of zinc transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of zinc transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of major facilitator superfamily transporter protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of major facilitator superfamily transporter protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of major facilitator superfamily transporter protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating one or more activities selected from the group consisting of DNA helicase IV in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of methylglyoxal synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of phosphoanhydride phosphorylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of b1003-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of b1003-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of b1003-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of b1163-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of b1163-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of b1163-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating one or more activities selected from the group consisting of DNA polymerase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of DNA polymerase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of membrane protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of membrane protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of membrane protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of anthranilate synthase component II in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of anthranilate synthase component II in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of anthranilate synthase component II in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating one or more activities selected from the group consisting of glutamine synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of aldehyde dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of b1445-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of NADP-dependent malic enzyme in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of b1522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of b1522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of b1522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of acid shock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of acid shock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of acid shock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of b1670-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of b1672-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of cytochrome c-type protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of trehalose-phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of trehalose-phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of trehalose-phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of O-antigen chain length determinant in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of O-antigen chain length determinant in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of GDP-mannose 4,6-dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of Nucleoside monophosphate phosphohydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of b2513-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of b2513-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of b2513-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of serine hydroxymethyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating one or more activities selected from the group consisting of b2613-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of b2673-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating one or more activities selected from the group consisting of murein transglycosylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of murein transglycosylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of murein transglycosylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of b2739-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of b2739-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of b2739-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of b2812-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of b2812-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of ribosephosphate isomerase, constitutive in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating one or more activities selected from the group consisting of arginine exporter protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of glycoprotease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of glycoprotease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of glycoprotease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of b3083-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating one or more activities selected from the group consisting of b3083-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating one or more activities selected from the group consisting of L-serine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of threonine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating one or more activities selected from the group consisting of acetyl CoA carboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of ABC transporter ATP-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of glycogen synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of glycogen synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of b3490-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of valine-pyruvate transaminase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of valine-pyruvate transaminase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of phosphopantetheine adenylyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of phosphopantetheine adenylyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of phosphopantetheine adenylyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating one or more activities selected from the group consisting of acetolactate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of branched-chain amino-acid aminotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating one or more activities selected from the group consisting of dihydroxyacid dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of dihydroxyacid dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of leucine, which comprises
(a) increasing or generating one or more activities selected from the group consisting of dihydroxyacid dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of dihydroxyacid dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of ketol-acid reductoisomerase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of ketol-acid reductoisomerase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of DNA helicase II in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of DNA helicase II in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of DNA helicase II in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating one or more activities selected from the group consisting of protoporphyrin oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of leucine, which comprises
(a) increasing or generating one or more activities selected from the group consisting of superoxide dismutase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of b3989-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of b3989-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of b3989-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of b4029-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of b4029-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of b4029-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of b4050-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating one or more activities selected from the group consisting of b4056-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of b4056-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of b4263-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of gluconate transport system permease 3 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of gluconate transport system permease 3 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of gluconate transport system permease 3 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of purine-nucleoside phosphorylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating one or more activities selected from the group consisting of thioredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of thioredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of thioredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of GM02LC11114-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of GM02LC11114-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of histone H2A in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of histone H2A in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of histone H2A in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of vacuolar ATPase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of RNA binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of RNA binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of RNA binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating one or more activities selected from the group consisting of GM02LC5744-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of agmatinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of ABC transporter ATP binding component in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of flavodoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of flavodoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of polyphosphate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of polyphosphate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of polyphosphate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of bifunctional protein (phosphoribosyltransferase and regulatory protein) in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of urease subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating one or more activities selected from the group consisting of NAD(P)H-quinone oxidoreductase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of oxireductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating one or more activities selected from the group consisting of binding-protein-dependent transport systems inner membrane component in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating one or more activities selected from the group consisting of sll1032-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of glycogen (starch) synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of glycogen (starch) synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of glycogen (starch) synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of sll1455-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of carbamoyl-phosphate synthase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of carbamoyl-phosphate synthase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of sll1761-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of sll1761-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of adenylate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of adenylate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of adenylate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of acyl transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating one or more activities selected from the group consisting of ferredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of precorrin methylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of precorrin methylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of precorrin methylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of malate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating one or more activities selected from the group consisting of malate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of malate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of leucine, which comprises
(a) increasing or generating one or more activities selected from the group consisting of malate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of malate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of malate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of geranylgeranyl pyrophosphate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of geranylgeranyl pyrophosphate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of geranylgeranyl pyrophosphate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of iron(III) dicitrate-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating one or more activities selected from the group consisting of Na+/K+ transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of Na+/K+ transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of 3-isopropylmalate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of photosystem II protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of photosystem II protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of photosystem II protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of phosphoadenosine phosphosulfate reductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of riboflavin biosynthesis protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of threonine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating one or more activities selected from the group consisting of threonine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of threonine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of leucine, which comprises
(a) increasing or generating one or more activities selected from the group consisting of threonine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of threonine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of threonine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of short-chain alcohol dehydrogenase family in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of Sec-independent protein translocase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of Sec-independent protein translocase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of Sec-independent protein translocase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of 2-isopropylmalate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of 2-isopropylmalate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of TTC1386-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of TTC1386-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of TTC1386-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of homocitrate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of homocitrate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of homocitrate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating one or more activities selected from the group consisting of YAL013W-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of cell division control protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of phosphoglycerate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of phosphoglycerate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of branched-chain amino acid permease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating one or more activities selected from the group consisting of branched-chain amino acid permease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating one or more activities selected from the group consisting of ydr183w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of hexose transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating one or more activities selected from the group consisting of ygl237c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of ygl237c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of leucine, which comprises
(a) increasing or generating one or more activities selected from the group consisting of YGR193C-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of yhr006w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of yhr006w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of H/ACA ribonucleoprotein complex subunit 3 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of H/ACA ribonucleoprotein complex subunit 3 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of glycerol-3-phosphate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of glycerol-3-phosphate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of homoserine dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating one or more activities selected from the group consisting of branched chain aminotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating one or more activities selected from the group consisting of branched chain aminotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of polygalacturonase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of poly-galacturonase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of poly-galacturonase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating one or more activities selected from the group consisting of YKL038W-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of YKL038W-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of YLR224W-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating one or more activities selected from the group consisting of mitochondrial dicarboxylate transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating one or more activities selected from the group consisting of adenylosuccinate lyase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of adenylosuccinate lyase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of adenylosuccinate lyase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating one or more activities selected from the group consisting of phosphate transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of acetyl-CoA acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of acetyl-CoA acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating one or more activities selected from the group consisting of cyclin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating one or more activities selected from the group consisting of Zm_{—}4842_BE510522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating one or more activities selected from the group consisting of Zm_{—}4842_BE510522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating one or more activities selected from the group consisting of Zm_{—}4842_BE510522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

An embodiment of the present invention relates to a process for the production of isoleucine, which comprises
(a1) increasing or generating the activity of a polypeptide comprising a polypeptide as depicted in a respective line in column 5 or 8 of Table II, application no. 6, or a homolog or a fragment thereof, a consensus sequence or at least one polypeptide motif as depicted in the respective line in column 8 of Table IV, application no. 6, whereby the respective line discloses in column 7 the fine chemical isoleucine; or (a2) increasing or generating the activity of an expression product of one or more nucleic acid molecule(s) comprising a polynucleotide as depicted in the respective line in column 5 or 8 of Table I, application no. 6, preferably the coding region thereof, or a homolog or a fragment thereof, whereby the respective line discloses in column 7 the fine chemical isoleucine; or (a3) increasing or generating the activity of a functional equivalent of (a1) or (a2); in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of isoleucine or a composition comprising isoleucine in said non-human organism or in the culture medium surrounding said non-human organism.

An embodiment of the present invention relates to a process for the production of leucine, which comprises
(a1) increasing or generating the activity of a polypeptide comprising a polypeptide as depicted in a respective line in column 5 or 8 of Table II, application no. 6, or a homolog or a fragment thereof, a consensus sequence or at least one polypeptide motif as depicted in the respective line in column 8 of Table IV, application no. 6, whereby the respective line discloses in column 7 the fine chemical leucine; or
(a2) increasing or generating the activity of an expression product of one or more nucleic acid molecule(s) comprising a polynucleotide as depicted in the respective line in column 5 or 8 of Table I, application no. 6, preferably the coding region thereof, or a homolog or a fragment thereof, whereby the respective line discloses in column 7 the fine chemical leucine; or
(a3) increasing or generating the activity of a functional equivalent of (a1) or (a2); in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of leucine or a composition comprising leucine in said non-human organism or in the culture medium surrounding said non-human organism.

An embodiment of the present invention relates to a process for the production of valine, which comprises
(a1) increasing or generating the activity of a polypeptide comprising a polypeptide as depicted in a respective line in column 5 or 8 of Table II, application no. 6, or a homolog or a fragment thereof, a consensus sequence or at least one polypeptide motif as depicted in the respective line in column 8 of Table IV, application no. 6, whereby the respective line discloses in column 7 the fine chemical valine; or
(a2) increasing or generating the activity of an expression product of one or more nucleic acid molecule(s) comprising a polynucleotide as depicted in the respective line in column 5 or 8 of Table I, application no. 6, preferably the coding region thereof, or a homolog or a fragment thereof, whereby the respective line discloses in column 7 the fine chemical valine; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of valine or a composition comprising valine in said non-human organism or in the culture medium surrounding said non-human organism.

Accordingly, the increase or generation of one or more said activities is for example conferred by one or more expression products of said nucleic acid molecule, e.g. proteins. Accordingly, in the present invention described above, the increase or generation of one or more said activities is for example conferred by one or more protein(s) each comprising a polypeptide selected from the group as depicted in Table II, application no. 6, column 5 and 8, or a homolog or a fragment thereof.

The process of the invention comprises in one embodiment the following steps:
(i) increasing or generating of the expression of; and/or
(ii) increasing or generating the expression of an expression product of; and/or
(iii) increasing or generating one or more activities of an expression product encoded by;
   at least one nucleic acid molecule (in the following “Fine Chemical Related Protein (FCRP)”-encoding gene or “FCRP”-gene) comprising a nucleic acid molecule selected from the group consisting of:
(a) a nucleic acid molecule encoding the polypeptide shown in column 5 or 8 of Table II, preferably Table II B, application no. 6, or a homolog or a fragment thereof;
(b) a nucleic acid molecule shown in column 5 or 8 of Table I, preferably Table I B, application no. 6, or a homolog or a fragment thereof (preferably the coding region thereof);
(c) a nucleic acid molecule, which, as a result of the degeneracy of the genetic code, can be derived from a polypeptide sequence depicted in column 5 or 8 of Table II, preferably Table II B, application no. 6;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 6, or the coding region thereof;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 6;
(i) a nucleic acid molecule encoding a polypeptide having the activity represented by a respective protein as depicted in column 5 of Table II, application no. 6;
(j) a nucleic acid molecule which comprises a polynucleotide, which is obtained by amplifying a cDNA library or a genomic library using the primers as depicted in column 8 of Table III, application no. 6; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt
of a nucleic acid molecule complementary to a nucleic acid molecule sequence characterized in (a), (b), (c), (d), (e), (f), (g), (h), (i) or (j); or a nucleic acid molecule comprising a sequence which is complementary thereto.
   (All the references to Tables I to IV are references to the corresponding lines of Tables I to IV.)

The term “increasing or generating of the expression of” may also be interchanged by the term “overexpressing”.

In a preferred embodiment thereof said nucleic acid molecule encodes a polypeptide which has the activity of the polypeptide represented by a protein comprising a polypeptide as depicted in the corresponding hit in column 5 of Table II, application no. 6.

In another preferred embodiment thereof said nucleic acid molecule confers a generation or an increase of the respective fine chemical in a non-human organism or a part thereof as compared to a corresponding wild-type non-human organism or part thereof.

In another preferred embodiment thereof said nucleic acid molecule encodes a polypeptide which has the activity of the polypeptide represented by a protein as depicted in the corresponding hit in column 5 of Table II, application no. 6, and confers a generation or an increase of the respective fine chemical in a non-human organism or a part thereof as compared to a corresponding wild-type non-human organism or part thereof.

Accordingly, the genes of the present invention or used in accordance with the present invention, which respectively encode a protein having an activity of 2,3-dihydroxyphenylpropionate 1,2-dioxygenase, 2-isopropylmalate synthase, 3-isopropylmalate dehydrogenase, 47266012-protein, 50S ribosomal protein L14, ABC transporter ATP binding component, ABC transporter ATP-binding protein, ABC transporter component, ABC transporter permease protein, acetolactate synthase, acetolactate synthase small subunit, acetyl CoA carboxylase, acetyl-CoA acetyltransferase, acetyltransferase, acid shock protein, acyl transferase, acyltransferase, adenylate kinase, adenylosuccinate lyase, agmatinase, aldehyde dehydrogenase, aminotransferase, anthranilate synthase component II, arginine exporter protein, At1g19800-protein, At1g23110-protein, At1g47380-protein, At4g32480-protein, At5g16650-protein, ATP-binding component of a transport system, auxin response factor, b0050-protein, b1003-protein, b1163-protein, b1445-protein, b1522-protein, b1670-protein, b1672-protein, b2513-protein, b2613-protein, b2673-protein, b2739-protein, b2812-protein, b3083-protein, b3490-protein, b3989-protein, b4029-protein, b4050-protein, b4056-protein, b4263-protein, beta-galactosidase, beta-hydroxylase, bifunctional protein (phosphoribosyltransferase and regulatory protein), binding-protein-dependent transport systems inner membrane component, branched chain aminotransferase, branched-chain amino acid permease, branched-chain amino-acid aminotransferase, calcium-dependent protein kinase, carbamoyl-phosphate synthase subunit, CBL-interacting protein kinase, cell division control protein, chaperone protein CIpB, cullin, cyclin, cyclin D, cytochrome c-type protein, dihydroxyacid dehydratase, disease resistance protein, DNA helicase II, DNA helicase IV, DNA mismatch repair protein, DNA polymerase, DNA-binding protein, elongation factor Tu, enoyl-CoA hydratase, eukaryotic translation initiation factor, eukaryotic translation initiation factor 5, ferredoxin, flavodoxin, fumarylacetoacetate hydrolase, GDP-mannose 4,6-dehydratase, geranylgeranyl pyrophosphate synthase, gluconate transport system permease 3, glucose-1-phosphate cytidylyltransferase, glucose-6-phosphate 1-dehydrogenase, glutamate-ammonia-ligase, glutamine synthetase, glutaredoxin, glycerol-3-phosphate dehydrogenase, glycogen (starch) synthase, glycogen synthase, glycoprotease, glycosyl transferase, GM02LC11114-protein, GM02LC5744-protein, H/ACA ribonucleoprotein complex subunit 3, harpin-induced family protein, heat shock transcription factor, hexose transporter, histone H2A, homocitrate synthase, homoserine dehydrogenase, hydrolase, iron(III) dicitrate-binding protein, isochorismate synthase, isopropylmalate isomerase large subunit, ketol-acid reductoisomerase, L-serine dehydratase, MADS-box transcription factor, major facilitator superfamily transporter protein, malate dehydrogenase, malic enzyme, membrane protein, membrane transport protein, methylglyoxal synthase, mitochondrial dicarboxylate transporter, monothiol glutaredoxin, murein transglycosylase, Na+/K+ transporter, NAD(P)H-quinone oxidoreductase subunit, NADH-quinone oxidoreductase subunit, NADP-dependent malic enzyme, Nucleoside monophosphate phosphohydrolase, O-antigen chain length determinant, oxidoreductase, oxireductase, peptidase, phosphate transporter, phosphoadenosine phosphosulfate reductase , phosphoanhydride phosphorylase, phosphoglycerate kinase, phosphopantetheine adenylyltransferase, photosystem II protein, polygalacturonase, polyphosphate kinase, precorrin methylase, protein kinase, protein phosphatase, protoporphyrin oxidase, purine nucleoside phosphorylase, purine-nucleoside phosphorylase, pyruvate kinase, riboflavin biosynthesis protein, ribosephosphate isomerase, constitutive, RNA binding protein, RNA-binding protein, Sec-independent protein translocase subunit, serine hydroxymethyltransferase, short-chain alcohol dehydrogenase family, sll1032-protein, sll1455-protein, sll1761-protein, superoxide dismutase, thioredoxin, thioredoxin family protein, threonine dehydratase, threonine synthase, transcription factor, transcriptional regulator, transport protein, trehalose phosphatase, TTC1386-protein, urease subunit, vacuolar ATPase subunit, valine-pyruvate transaminase, YAL013W-protein, ydr183w-protein, ygl237c-protein, YGR193C-protein, yhr006w-protein, YKL038W-protein, YLR224W-protein, zinc finger protein, zinc transporter, or Zm_{—}4842_BE510522-protein, which respectively encode a protein comprising a polypeptide encoded by a nucleic acid sequence as shown in Table I, application no. 6, column 5 or 8, (preferably the coding region thereof), or a homolog or a fragment thereof, which respectively encode a protein comprising a polypeptide as depicted in Table II, application no. 6, column 5 or 8, or a homolg or a fragment thereof, and/or which respectively can be amplified with the primer set shown in Table III, application no. 6, column 8, are also referred to as “FCRP genes”.

Proteins or polypeptides encoded by “FCRP-genes” are referred to as “Fine Chemical Related Proteins” or “FCRP”. For the purposes of the description of the present invention, the respective protein having an activity of 2,3-dihydroxyphenylpropionate 1,2-dioxygenase, 2-isopropylmalate synthase, 3-isopropylmalate dehydrogenase, 47266012-protein, 50S ribosomal protein L14, ABC transporter ATP binding component, ABC transporter ATP-binding protein, ABC transporter component, ABC transporter permease protein, acetolactate synthase, acetolactate synthase small subunit, acetyl CoA carboxylase, acetyl-CoA acetyltransferase, acetyltransferase, acid shock protein, acyl transferase, acyltransferase, adenylate kinase, adenylosuccinate lyase, agmatinase, aldehyde dehydrogenase, aminotransferase, anthranilate synthase component II, arginine exporter protein, At1g19800-protein, At1g23110-protein, At1g47380-protein, At4g32480-protein, At5g16650-protein, ATP-binding component of a transport system, auxin response factor, b0050-protein, b1003-protein, b1163-protein, b1445-protein, b1522-protein, b1670-protein, b1672-protein, b2513-protein, b2613-protein, b2673-protein, b2739-protein, b2812-protein, b3083-protein, b3490-protein, b3989-protein, b4029-protein, b4050-protein, b4056-protein, b4263-protein, beta-galactosidase, beta-hydroxylase, bifunctional protein (phosphoribosyltransferase and regulatory protein), binding-proteindependent transport systems inner membrane component, branched chain aminotransferase, branched-chain amino acid permease, branched-chain amino-acid aminotransferase, calcium-dependent protein kinase, carbamoyl-phosphate synthase subunit, CBL-interacting protein kinase, cell division control protein, chaperone protein CIpB, cullin, cyclin, cyclin D, cytochrome c-type protein, dihydroxyacid dehydratase, disease resistance protein, DNA helicase II, DNA helicase IV, DNA mismatch repair protein, DNA polymerase, DNA-binding protein, elongation factor Tu, enoyl-CoA hydratase, eukaryotic translation initiation factor, eukaryotic translation initiation factor 5, ferredoxin, flavodoxin, fumarylacetoacetate hydrolase, GDP-mannose 4,6-dehydratase, geranylgeranyl pyrophosphate synthase, gluconate transport system permease 3, glucose-1-phosphate cytidylyltransferase, glucose-6-phosphate 1-dehydrogenase, glutamate-ammonia-ligase, glutamine synthetase, glutaredoxin, glycerol-3-phosphate dehydrogenase, glycogen (starch) synthase, glycogen synthase, glycoprotease, glycosyl transferase, GM02LC11114-protein, GM02LC5744-protein, H/ACA ribonucleoprotein complex subunit 3, harpin-induced family protein, heat shock transcription factor, hexose transporter, histone H2A, homocitrate synthase, homoserine dehydrogenase, hydrolase, iron(III) dicitrate-binding protein, isochorismate synthase, isopropylmalate isomerase large subunit, ketol-acid reductoisomerase, L-serine dehydratase, MADS-box transcription factor, major facilitator superfamily transporter protein, malate dehydrogenase, malic enzyme, membrane protein, membrane transport protein, methylglyoxal synthase, mitochondrial dicarboxylate transporter, monothiol glutaredoxin, murein transglycosylase, Na+/K+ transporter, NAD(P)H-quinone oxidoreductase subunit, NADH-quinone oxidoreductase subunit, NADP-dependent malic enzyme, Nucleoside monophosphate phosphohydrolase, O-antigen chain length determinant, oxidoreductase, oxireductase, peptidase, phosphate transporter, phosphoadenosine phosphosulfate reductase , phosphoanhydride phosphorylase, phosphoglycerate kinase, phosphopantetheine adenylyltransferase, photosystem II protein, polygalacturonase, polyphosphate kinase, precorrin methylase, protein kinase, protein phosphatase, protoporphyrin oxidase, purine nucleoside phosphorylase, purinenucleoside phosphorylase, pyruvate kinase, riboflavin biosynthesis protein, ribosephosphate isomerase, constitutive, RNA binding protein, RNA-binding protein, Sec-independent protein translocase subunit, serine hydroxymethyltransferase, short-chain alcohol dehydrogenase family, sll1032-protein, sll1455-protein, sll1761-protein, superoxide dismutase, thioredoxin, thioredoxin family protein, threonine dehydratase, threonine synthase, transcription factor, transcriptional regulator, transport protein, trehalose-phosphatase, TTC1386-protein, urease subunit, vacuolar ATPase subunit, valine-pyruvate transaminase, YAL013W-protein, ydr183w-protein, ygl237c-protein, YGR193C-protein, yhr006w-protein, YKL038W-protein, YLR224W-protein, zinc finger protein, zinc transporter, or Zm_{—}4842_BE510522-protein, the respective protein comprising a polypeptide encoded by one or more respective nucleic acid sequences as shown in Table I, application no. 6, column 5 or 8, (preferably the coding region thereof), or a homolog or fragment thereof, the respective protein comprising a respective polypeptide as depicted in Table II, application no. 6, column 5 or 8, or a homolog or fragment thereof, the respective protein comprising a sequence corresponding to the consensus sequence as shown in Table IV, application no. 6, column 8, and/or the respective protein comprising at least one polypeptide motif as shown in Table IV, application no. 6, column 8 is also referred to as Fine Chemical Related Protein” or “FCRP”.

Thus, in one embodiment, the present invention provides a process of the production of isoleucine, leucine, and/or valine, by increasing or generating one or more activities, especially selected from the group consisting of 2,3-dihydroxyphenylpropionate 1,2-dioxygenase, 2-isopropylmalate synthase, 3-isopropylmalate dehydrogenase, 47266012-protein, 50S ribosomal protein L14, ABC transporter ATP binding component, ABC transporter ATP-binding protein, ABC transporter component, ABC transporter permease protein, acetolactate synthase, acetolactate synthase small subunit, acetyl CoA carboxylase, acetyl-CoA acetyltransferase, acetyltransferase, acid shock protein, acyl transferase, acyltransferase, adenylate kinase, adenylosuccinate lyase, agmatinase, aldehyde dehydrogenase, aminotransferase, anthranilate synthase component II, arginine exporter protein, At1g19800-protein, At1g23110-protein, At1g47380-protein, At4g32480-protein, At5g16650-protein, ATP-binding component of a transport system, auxin response factor, b0050-protein, b1003-protein, b1163-protein, b1445-protein, b1522-protein, b1670-protein, b1672-protein, b2513-protein, b2613-protein, b2673-protein, b2739-protein, b2812-protein, b3083-protein, b3490-protein, b3989-protein, b4029-protein, b4050-protein, b4056-protein, b4263-protein, beta-galactosidase, beta-hydroxylase, bifunctional protein (phosphoribosyltransferase and regulatory protein), binding-protein-dependent transport systems inner membrane component, branched chain aminotransferase, branched-chain amino acid permease, branched-chain amino-acid aminotransferase, calcium-dependent protein kinase, carbamoyl-phosphate synthase subunit, CBL-interacting protein kinase, cell division control protein, chaperone protein CIpB, cullin, cyclin, cyclin D, cytochrome c-type protein, dihydroxyacid dehydratase, disease resistance protein, DNA helicase II, DNA helicase IV, DNA mismatch repair protein, DNA polymerase, DNA-binding protein, elongation factor Tu, enoyl-CoA hydratase, eukaryotic translation initiation factor, eukaryotic translation initiation factor 5, ferredoxin, flavodoxin, fumarylacetoacetate hydrolase, GDP-mannose 4,6-dehydratase, geranylgeranyl pyrophosphate synthase, gluconate transport system permease 3, glucose-1-phosphate cytidylyltransferase, glucose-6-phosphate 1-dehydrogenase, glutamate-ammonia-ligase, glutamine synthetase, glutaredoxin, glycerol-3-phosphate dehydrogenase, glycogen (starch) synthase, glycogen synthase, glycoprotease, glycosyl transferase, GM02LC11114-protein, GM02LC5744-protein, H/ACA ribonucleoprotein complex subunit 3, harpin-induced family protein, heat shock transcription factor, hexose transporter, histone H2A, homocitrate synthase, homoserine dehydrogenase, hydrolase, iron(III) dicitrate-binding protein, isochorismate synthase, isopropylmalate isomerase large subunit, ketol-acid reductoisomerase, L-serine dehydratase, MADS-box transcription factor, major facilitator superfamily transporter protein, malate dehydrogenase, malic enzyme, membrane protein, membrane transport protein, methylglyoxal synthase, mitochondrial dicarboxylate transporter, monothiol glutaredoxin, murein transglycosylase, Na+/K+ transporter, NAD(P)H-quinone oxidoreductase subunit, NADH-quinone oxidoreductase subunit, NADP-dependent malic enzyme, Nucleoside monophosphate phosphohydrolase, O-antigen chain length determinant, oxidoreductase, oxireductase, peptidase, phosphate transporter, phosphoadenosine phosphosulfate reductase , phosphoanhydride phosphorylase, phosphoglycerate kinase, phosphopantetheine adenylyltransferase, photosystem II protein, polygalacturonase, polyphosphate kinase, precorrin methylase, protein kinase, protein phosphatase, protoporphyrin oxidase, purine nucleoside phosphorylase, purinenucleoside phosphorylase, pyruvate kinase, riboflavin biosynthesis protein, ribosephosphate isomerase, constitutive, RNA binding protein, RNA-binding protein, Sec-independent protein translocase subunit, serine hydroxymethyltransferase, short-chain alcohol dehydrogenase family, sll1032-protein, sll1455-protein, sll1761-protein, superoxide dismutase, thioredoxin, thioredoxin family protein, threonine dehydratase, threonine synthase, transcription factor, transcriptional regulator, transport protein, trehalose-phosphatase, TTC1386-protein, urease subunit, vacuolar ATPase subunit, valine-pyruvate transaminase, YAL013W-protein, ydr183w-protein, ygl237c-protein, YGR193C-protein, yhr006w-protein, YKL038W-protein, YLR224W-protein, zinc finger protein, zinc transporter, and Zm_{—}4842_BE510522-protein, which is conferred by one or more FCRPs or the gene product of one or more FCRP-genes, for example by the gene product of a nucleic acid sequences comprising a polynucleotide selected from the group as shown in Table I, application no. 6, column 5 or 8, (preferably by the coding region thereof), or a homolog or a fragment thereof, e.g. or by one or more proteins each comprising a polypeptide encoded by one or more nucleic acid sequences selected from the group as shown in Table I, application no. 6, column 5 or 8, (preferably by the coding region thereof), or a homolog or a fragment thereof, or by one or more protein(s) each comprising a polypeptide selected from the group as depicted in Table II, application no. 6, column 5 and 8, or a homolog thereof, or a protein comprising a sequence corresponding to the consensus sequence or comprising at least one polypeptide motif as shown in Table IV, application no. 6, column 8.

for the disclosure of this paragraph see [0025.1.1.1] above.

In an embodiment, the process comprises increasing or generating the activity of one or more polypeptides having said activity, e.g. by generating or increasing the amount and/or specific activity in the cell or a compartment of a cell of one of more FCRP, especially selected from the group consisting of 2,3-dihydroxyphenylpropionate 1,2-dioxygenase, 2-isopropylmalate synthase, 3-isopropylmalate dehydrogenase, 47266012-protein, 50S ribosomal protein L14, ABC transporter ATP binding component, ABC transporter ATP-binding protein, ABC transporter component, ABC transporter permease protein, acetolactate synthase, acetolactate synthase small subunit, acetyl CoA carboxylase, acetyl-CoA acetyltransferase, acetyltransferase, acid shock protein, acyl transferase, acyltransferase, adenylate kinase, adenylosuccinate lyase, agmatinase, aldehyde dehydrogenase, aminotransferase, anthranilate synthase component II, arginine exporter protein, At1g19800-protein, At1g23110-protein, At1g47380-protein, At4g32480-protein, At5g16650-protein, ATP-binding component of a transport system, auxin response factor, b0050-protein, b1003-protein, b1163-protein, b1445-protein, b1522-protein, b1670-protein, b1672-protein, b2513-protein, b2613-protein, b2673-protein, b2739-protein, b2812-protein, b3083-protein, b3490-protein, b3989-protein, b4029-protein, b4050-protein, b4056-protein, b4263-protein, beta-galactosidase, beta-hydroxylase, bifunctional protein (phosphoribosyltransferase and regulatory protein), binding-protein-dependent transport systems inner membrane component, branched chain aminotransferase, branched-chain amino acid permease, branched-chain amino-acid aminotransferase, calcium-dependent protein kinase, carbamoyl-phosphate synthase subunit, CBL-interacting protein kinase, cell division control protein, chaperone protein CIpB, cullin, cyclin, cyclin D, cytochrome c-type protein, dihydroxyacid dehydratase, disease resistance protein, DNA helicase II, DNA helicase IV, DNA mismatch repair protein, DNA polymerase, DNA-binding protein, elongation factor Tu, enoyl-CoA hydratase, eukaryotic translation initiation factor, eukaryotic translation initiation factor 5, ferredoxin, flavodoxin, fumarylacetoacetate hydrolase, GDP-mannose 4,6-dehydratase, geranylgeranyl pyrophosphate synthase, gluconate transport system permease 3, glucose-1-phosphate cytidylyltransferase, glucose-6-phosphate 1-dehydrogenase, glutamate-ammonia-ligase, glutamine synthetase, glutaredoxin, glycerol-3-phosphate dehydrogenase, glycogen (starch) synthase, glycogen synthase, glycoprotease, glycosyl transferase, GM02LC11114-protein, GM02LC5744-protein, H/ACA ribonucleoprotein complex subunit 3, harpin-induced family protein, heat shock transcription factor, hexose transporter, histone H2A, homocitrate synthase, homoserine dehydrogenase, hydrolase, iron(III) dicitrate-binding protein, isochorismate synthase, isopropylmalate isomerase large subunit, ketol-acid reductoisomerase, L-serine dehydratase, MADS-box transcription factor, major facilitator superfamily transporter protein, malate dehydrogenase, malic enzyme, membrane protein, membrane transport protein, methylglyoxal synthase, mitochondrial dicarboxylate transporter, monothiol glutaredoxin, murein transglycosylase, Na+/K+ transporter, NAD(P)H-quinone oxidoreductase subunit, NADH-quinone oxidoreductase subunit, NADP-dependent malic enzyme, Nucleoside monophosphate phosphohydrolase, O-antigen chain length determinant, oxidoreductase, oxireductase, peptidase, phosphate transporter, phosphoadenosine phosphosulfate reductase , phosphoanhydride phosphorylase, phosphoglycerate kinase, phosphopantetheine adenylyltransferase, photosystem II protein, polygalacturonase, polyphosphate kinase, precorrin methylase, protein kinase, protein phosphatase, protoporphyrin oxidase, purine nucleoside phosphorylase, purine-nucleoside phosphorylase, pyruvate kinase, riboflavin biosynthesis protein, ribosephosphate isomerase, constitutive, RNA binding protein, RNA-binding protein, Sec-independent protein translocase subunit, serine hydroxymethyltransferase, short-chain alcohol dehydrogenase family, sll1032-protein, sll1455-protein, sll1761protein, superoxide dismutase, thioredoxin, thioredoxin family protein, threonine dehydratase, threonine synthase, transcription factor, transcriptional regulator, transport protein, trehalosephosphatase, TTC1386-protein, urease subunit, vacuolar ATPase subunit, valine-pyruvate transaminase, YAL013W-protein, ydr183w-protein, ygl237c-protein, YGR193C-protein, yhr006w-protein, YKL038W-protein, YLR224W-protein, zinc finger protein, zinc transporter, and Zm_{—}4842_BE510522-protein, for example of the respective polypeptide as depicted in Table II, application no. 6, column 5 and 8, or a homolog or a fragment thereof, or the respective polypeptide comprising a sequence corresponding to the consensus sequences as shown in Table IV, application no. 6, column 8, or the respective polypeptide comprising at least one polypeptide motif as depicted in Table IV, application no. 6, column 8.

A further embodiment of the present invention relates to a process for the production of isoleucine, which comprises
(a) increasing or generating the activity of a 47266012-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a glucose-6-phosphate 1-dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a glucose-6-phosphate 1-dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a glucose-6-phosphate 1-dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a DNA-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a At1g19800-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a At1g19800-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a At1g23110-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a cullin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a cullin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a eukaryotic translation initiation factor 5 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a eukaryotic translation initiation factor 5 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating the activity of a At1g47380-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a At1g47380-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a CBL-interacting protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a CBL-interacting protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a CBL-interacting protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a thioredoxin family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a thioredoxin family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a harpin-induced family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a harpin-induced family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a DNA mismatch repair protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a DNA mismatch repair protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a DNA mismatch repair protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a eukaryotic translation initiation factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a eukaryotic translation initiation factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a eukaryotic translation initiation factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a glycosyl transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a glycosyl transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a glycosyl transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a auxin response factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a auxin response factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a auxin response factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a At4g32480-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a At4g32480-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a At4g32480-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating the activity of a At5g16650-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating the activity of a At5g16650-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating the activity of a thioredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a thioredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a disease resistance protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a RNA-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a MADS-box transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a MADS-box transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a zinc finger protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a malic enzyme non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a malic enzyme non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a malic enzyme non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a beta-hydroxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a elongation factor Tu non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a elongation factor Tu non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a elongation factor Tu non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a 50S ribosomal protein L14 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a enoyl-CoA hydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a enoyl-CoA hydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a enoyl-CoA hydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a acyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a acyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a acyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a ABC transporter component non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a glucose-1-phosphate cytidylyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a aminotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating the activity of a aminotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a NADH-quinone oxidoreductase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a NADH-quinone oxidoreductase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a glutamate-ammonia-ligase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a glutamate-ammonia-ligase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a glutamate-ammonia-ligase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a isopropylmalate isomerase large subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a fumarylacetoacetate hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a fumarylacetoacetate hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a fumarylacetoacetate hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating the activity of a peptidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a chaperone protein CIpB non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating the activity of a purine nucleoside phosphorylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a purine nucleoside phosphorylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a purine nucleoside phosphorylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a threonine synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a threonine synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a threonine synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a b0050-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating the activity of a acetolactate synthase small subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of leucine, which comprises
(a) increasing or generating the activity of a beta-galactosidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a 2,3-dihydroxyphenylpropionate 1,2-dioxygenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating the activity of a 2,3-dihydroxyphenylpropionate 1,2-dioxygenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a ATP-binding component of a transport system non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a ATP-binding component of a transport system non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a ATP-binding component of a transport system non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a membrane transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating the activity of a isochorismate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a isochorismate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a isochorismate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a zinc transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a zinc transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a major facilitator superfamily transporter protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a major facilitator superfamily transporter protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a major facilitator superfamily transporter protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating the activity of a DNA helicase IV non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a methylglyoxal synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a phosphoanhydride phosphorylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a b1003-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a b1003-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a b1003-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a b1163-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a b1163-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a b1163-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating the activity of a DNA polymerase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a DNA polymerase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a membrane protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a membrane protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a membrane protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a anthranilate synthase component II non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a anthranilate synthase component II non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a anthranilate synthase component II non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating the activity of a glutamine synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a aldehyde dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a b1445-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a NADP-dependent malic enzyme non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a b1522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a b1522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a b1522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a acid shock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a acid shock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a acid shock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a b1670-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a b1672-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a cytochrome c-type protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a trehalose-phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a trehalose-phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a trehalose-phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a O-antigen chain length determinant non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a O-antigen chain length determinant non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a GDP-mannose 4,6-dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a Nucleoside monophosphate phosphohydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a b2513-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a b2513-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a b2513-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a serine hydroxymethyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating the activity of a b2613-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a b2673-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating the activity of a murein transglycosylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a murein transglycosylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a murein transglycosylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a b2739-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a b2739-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a b2739-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a b2812-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a b2812-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a ribosephosphate isomerase, constitutive non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating the activity of a arginine exporter protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a glycoprotease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a glycoprotease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a glycoprotease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a b3083-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating the activity of a b3083-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating the activity of a L-serine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a threonine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating the activity of a acetyl CoA carboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a ABC transporter ATP-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a glycogen synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a glycogen synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a b3490-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a valine-pyruvate transaminase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a valine-pyruvate transaminase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a phosphopantetheine adenylyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a phosphopantetheine adenylyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a phosphopantetheine adenylyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating the activity of a acetolactate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a branched-chain amino-acid aminotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating the activity of a dihydroxyacid dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a dihydroxyacid dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of leucine, which comprises
(a) increasing or generating the activity of a dihydroxyacid dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a dihydroxyacid dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a ketol-acid reductoisomerase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a ketol-acid reductoisomerase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a DNA helicase II non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a DNA helicase II non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a DNA helicase II non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating the activity of a protoporphyrin oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of leucine, which comprises
(a) increasing or generating the activity of a superoxide dismutase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a b3989-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a b3989-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a b3989-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a b4029-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a b4029-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a b4029-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a b4050-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating the activity of a b4056-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a b4056-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a b4263-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a gluconate transport system permease 3 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a gluconate transport system permease 3 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a gluconate transport system permease 3 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a purine-nucleoside phosphorylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating the activity of a thioredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a thioredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a thioredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a GM02LC11114-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a GM02LC11114-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a histone H2A non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a histone H2A non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a histone H2A non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a vacuolar ATPase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a RNA binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a RNA binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a RNA binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating the activity of a GM02LC5744-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a agmatinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a ABC transporter ATP binding component non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a flavodoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a flavodoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a polyphosphate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a polyphosphate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a polyphosphate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a bifunctional protein (phosphoribosyltransferase and regulatory protein) non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a urease subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating the activity of a NAD(P)H-quinone oxidoreductase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a oxireductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating the activity of a binding-protein-dependent transport systems inner membrane component non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating the activity of a sll1032-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a glycogen (starch) synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a glycogen (starch) synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a glycogen (starch) synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a sll1455-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a carbamoyl-phosphate synthase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a carbamoyl-phosphate synthase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a sll1761-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a sll1761-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a adenylate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a adenylate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a adenylate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a acyl transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating the activity of a ferredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a precorrin methylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a precorrin methylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a precorrin methylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a malate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating the activity of a malate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a malate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of leucine, which comprises
(a) increasing or generating the activity of a malate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a malate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a malate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a geranylgeranyl pyrophosphate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a geranylgeranyl pyrophosphate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a geranylgeranyl pyrophosphate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a iron(III) dicitrate-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating the activity of a Na+/K+ transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a Na+/K+ transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a 3-isopropylmalate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a photosystem II protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a photosystem II protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a photosystem II protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a phosphoadenosine phosphosulfate reductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a riboflavin biosynthesis protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a threonine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating the activity of a threonine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a threonine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of leucine, which comprises
(a) increasing or generating the activity of a threonine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a threonine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a threonine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a short-chain alcohol dehydrogenase family non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a Sec-independent protein translocase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a Sec-independent protein translocase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a Sec-independent protein translocase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a 2-isopropylmalate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a 2-isopropylmalate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a TTC1386-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a TTC1386-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a TTC1386-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a homocitrate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a homocitrate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a homocitrate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating the activity of a YAL013W-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a cell division control protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a phosphoglycerate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a phosphoglycerate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a branched-chain amino acid permease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating the activity of a branched-chain amino acid permease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating the activity of a ydr183w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a hexose transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating the activity of a ygl237c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a ygl237c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of leucine, which comprises
(a) increasing or generating the activity of a YGR193C-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a yhr006w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a yhr006w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a H/ACA ribonucleoprotein complex subunit 3 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a H/ACA ribonucleoprotein complex subunit 3 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a glycerol-3-phosphate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a glycerol-3-phosphate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a homoserine dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating the activity of a branched chain aminotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating the activity of a branched chain aminotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a polygalacturonase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a polygalacturonase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a polygalacturonase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a YKL038W-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a YKL038W-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a YLR224W-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating the activity of a mitochondrial dicarboxylate transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating the activity of a adenylosuccinate lyase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a adenylosuccinate lyase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a adenylosuccinate lyase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating the activity of a phosphate transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a acetyl-CoA acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a acetyl-CoA acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating the activity of a cyclin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating the activity of a Zm_{—}4842_BE510522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating the activity of a Zm_{—}4842_BE510522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating the activity of a Zm_{—}4842_BE510522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating the activity of a polypeptide comprising a polypeptide as depicted in the respective line in column 5 or 8 of Table II, application no. 6, or a homolog or a fragment thereof, a consensus sequence or at least one polypeptide motif as depicted in the respective line in column 8 of Table IV, application no. 6, whereby the respective line discloses in column 7 the fine chemical isoleucine; or
increasing or generating the activity of an expression product of one or more nucleic acid molecule(s) comprising a polynucleotide as depicted in the respective line in column 5 or 8 of Table I, application no. 6, preferably the coding region thereof, or a homolog or a fragment thereof, whereby the respective line discloses in column 7 the fine chemical isoleucine;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of isoleucine, or a composition comprising isoleucine in said non-human organism or in the culture medium surrounding said non-human organism.

A further embodiment of the present invention relates to a process for the production of leucine, which comprises
(a) increasing or generating the activity of a polypeptide comprising a polypeptide as depicted in the respective line in column 5 or 8 of Table II, application no. 6, or a homolog or a fragment thereof, a consensus sequence or at least one polypeptide motif as depicted in the respective line in column 8 of Table IV, application no. 6, whereby the respective line discloses in column 7 the fine chemical leucine; or
increasing or generating the activity of an expression product of one or more nucleic acid molecule(s) comprising a polynucleotide as depicted in the respective line in column 5 or 8 of Table I, application no. 6, preferably the coding region thereof, or a homolog or a fragment thereof, whereby the respective line discloses in column 7 the fine chemical leucine;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of leucine, or a composition comprising leucine in said non-human organism or in the culture medium surrounding said non-human organism.

A further embodiment of the present invention relates to a process for the production of valine, which comprises
(a) increasing or generating the activity of a polypeptide comprising a polypeptide as depicted in the respective line in column 5 or 8 of Table II, application no. 6, or a homolog or a fragment thereof, a consensus sequence or at least one polypeptide motif as depicted in the respective line in column 8 of Table IV, application no. 6, whereby the respective line discloses in column 7 the fine chemical valine; or
increasing or generating the activity of an expression product of one or more nucleic acid molecule(s) comprising a polynucleotide as depicted in the respective line in column 5 or 8 of Table I, application no. 6, preferably the coding region thereof, or a homolog or a fragment thereof, whereby the respective line discloses in column 7 the fine chemical valine;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of valine, or a composition comprising valine in said non-human organism or in the culture medium surrounding said non-human organism.

A further embodiment of the present invention relates to a process for the production of isoleucine, which comprises
(a) increasing or generating one or more activities selected from the group consisting of 47266012-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of glucose-6-phosphate 1-dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of glucose-6-phosphate 1-dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of glucose-6-phosphate 1-dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of DNA-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of At1g19800-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of At1g19800-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of At1g23110-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of cullin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of cullin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of eukaryotic translation initiation factor 5 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of eukaryotic translation initiation factor 5 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating one or more activities selected from the group consisting of At1g47380-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of At1g47380-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of CBL-interacting protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of CBL-interacting protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of CBL-interacting protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of thioredoxin family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of thioredoxin family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of harpin-induced family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of harpin-induced family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of DNA mismatch repair protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of DNA mismatch repair protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of DNA mismatch repair protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of eukaryotic translation initiation factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of eukaryotic translation initiation factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of eukaryotic translation initiation factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of glycosyl transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of glycosyl transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of glycosyl transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of auxin response factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of auxin response factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of auxin response factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of At4g32480-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of At4g32480-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of At4g32480-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating one or more activities selected from the group consisting of At5g16650-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating one or more activities selected from the group consisting of At5g16650-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating one or more activities selected from the group consisting of thioredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of thioredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of disease resistance protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of RNA-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of MADS-box transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of MADS-box transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of zinc finger protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of malic enzyme in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of malic enzyme in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of malic enzyme in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of betahydroxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of elongation factor Tu in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of elongation factor Tu in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of elongation factor Tu in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of 50S ribosomal protein L14 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of enoyl-CoA hydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of enoyl-CoA hydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of enoyl-CoA hydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of acyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of acyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of acyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of ABC transporter component in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of glucose-1-phosphate cytidylyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of aminotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating one or more activities selected from the group consisting of aminotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of NADH-quinone oxidoreductase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of NADH-quinone oxidoreductase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of glutamate-ammonia-ligase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of glutamate-ammonia-ligase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of glutamate-ammonia-ligase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of isopropylmalate isomerase large subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of fumarylacetoacetate hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of fumarylacetoacetate hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of fumarylacetoacetate hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating one or more activities selected from the group consisting of peptidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of chaperone protein CIpB in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating one or more activities selected from the group consisting of purine nucleoside phosphorylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of purine nucleoside phosphorylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of purine nucleoside phosphorylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of threonine synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of threonine synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of threonine synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of b0050-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating one or more activities selected from the group consisting of acetolactate synthase small subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of leucine, which comprises
(a) increasing or generating one or more activities selected from the group consisting of betagalactosidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of 2,3-dihydroxyphenylpropionate 1,2-dioxygenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating one or more activities selected from the group consisting of 2,3-dihydroxyphenylpropionate 1,2-dioxygenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of ATP-binding component of a transport system in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of ATP-binding component of a transport system in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of ATP-binding component of a transport system in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of membrane transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating one or more activities selected from the group consisting of isochorismate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of isochorismate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of isochorismate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of zinc transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of zinc transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of major facilitator superfamily transporter protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of major facilitator superfamily transporter protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of major facilitator superfamily transporter protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating one or more activities selected from the group consisting of DNA helicase IV in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of methylglyoxal synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of phosphoanhydride phosphorylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of b1003-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of b1003-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of b1003-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of b1163-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of b1163-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of b1163-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating one or more activities selected from the group consisting of DNA polymerase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of DNA polymerase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of membrane protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of membrane protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of membrane protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of anthranilate synthase component II in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of anthranilate synthase component II in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of anthranilate synthase component II in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating one or more activities selected from the group consisting of glutamine synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of aldehyde dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of b1445-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of NADP-dependent malic enzyme in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of b1522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of b1522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of b1522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of acid shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of acid shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of acid shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of b1670-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of b1672-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of cytochrome c-type protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of trehalose-phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of trehalose-phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of trehalose-phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of O-antigen chain length determinant in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of O-antigen chain length determinant in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of GDP-mannose 4,6-dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of Nucleoside monophosphate phosphohydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of b2513-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of b2513-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of b2513-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of serine hydroxymethyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating one or more activities selected from the group consisting of b2613-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of b2673-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating one or more activities selected from the group consisting of murein transglycosylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of murein transglycosylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of murein transglycosylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of b2739-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of b2739-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of b2739-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of b2812-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of b2812-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of ribosephosphate isomerase, constitutive in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating one or more activities selected from the group consisting of arginine exporter protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of glycoprotease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of glycoprotease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of glycoprotease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of b3083-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating one or more activities selected from the group consisting of b3083-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating one or more activities selected from the group consisting of L-serine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of threonine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating one or more activities selected from the group consisting of acetyl CoA carboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of ABC transporter ATP-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of glycogen synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of glycogen synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of b3490-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of valine-pyruvate transaminase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of valine-pyruvate transaminase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of phosphopantetheine adenylyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of phosphopantetheine adenylyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of phosphopantetheine adenylyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating one or more activities selected from the group consisting of acetolactate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of branched-chain amino-acid aminotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating one or more activities selected from the group consisting of dihydroxyacid dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of dihydroxyacid dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of leucine, which comprises
(a) increasing or generating one or more activities selected from the group consisting of dihydroxyacid dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of dihydroxyacid dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of ketol-acid reductoisomerase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of ketol-acid reductoisomerase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of DNA helicase II in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of DNA helicase II in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of DNA helicase II in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating one or more activities selected from the group consisting of protoporphyrin oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of leucine, which comprises
(a) increasing or generating one or more activities selected from the group consisting of superoxide dismutase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of b3989-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of b3989-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of b3989-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of b4029-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of b4029-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of b4029-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of b4050-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating one or more activities selected from the group consisting of b4056-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of b4056-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of b4263-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of gluconate transport system permease 3 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of gluconate transport system permease 3 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of gluconate transport system permease 3 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of purine-nucleoside phosphorylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating one or more activities selected from the group consisting of thioredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of thioredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of thioredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of GM02LC11114-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of GM02LC11114-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of histone H2A in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of histone H2A in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of histone H2A in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of vacuolar ATPase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of RNA binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of RNA binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of RNA binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating one or more activities selected from the group consisting of GM02LC5744-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of agmatinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of ABC transporter ATP binding component in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of flavodoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of flavodoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of polyphosphate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of polyphosphate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of polyphosphate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of bifunctional protein (phosphoribosyltransferase and regulatory protein) in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of urease subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating one or more activities selected from the group consisting of NAD(P)H-quinone oxidoreductase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of oxireductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating one or more activities selected from the group consisting of binding-protein-dependent transport systems inner membrane component in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating one or more activities selected from the group consisting of sll1032-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of glycogen (starch) synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of glycogen (starch) synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of glycogen (starch) synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of sll1455-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of carbamoyl-phosphate synthase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of carbamoyl-phosphate synthase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of sll1761-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of sll1761-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of adenylate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of adenylate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of adenylate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of acyl transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating one or more activities selected from the group consisting of ferredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of precorrin methylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of precorrin methylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of precorrin methylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of malate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating one or more activities selected from the group consisting of malate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of malate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of leucine, which comprises
(a) increasing or generating one or more activities selected from the group consisting of malate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of malate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of malate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of geranylgeranyl pyrophosphate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of geranylgeranyl pyrophosphate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of geranylgeranyl pyrophosphate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of iron(III) dicitrate-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating one or more activities selected from the group consisting of Na+/K+ transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of Na+/K+ transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of 3-isopropylmalate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of photosystem II protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of photosystem II protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of photosystem II protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of phosphoadenosine phosphosulfate reductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of riboflavin biosynthesis protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of threonine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating one or more activities selected from the group consisting of threonine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of threonine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of leucine, which comprises
(a) increasing or generating one or more activities selected from the group consisting of threonine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of threonine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of threonine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of short-chain alcohol dehydrogenase family in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of Sec-independent protein translocase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of Sec-independent protein translocase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of Sec-independent protein translocase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of 2-isopropylmalate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of 2-isopropylmalate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of TTC1386-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of TTC1386-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of TTC1386-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of homocitrate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of homocitrate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of homocitrate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating one or more activities selected from the group consisting of YAL013W-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of cell division control protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of phosphoglycerate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of phosphoglycerate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of branched-chain amino acid permease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating one or more activities selected from the group consisting of branched-chain amino acid permease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of valine, which comprises
(a) increasing or generating one or more activities selected from the group consisting of ydr183w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of hexose transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating one or more activities selected from the group consisting of ygl237c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of ygl237c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of leucine, which comprises
(a) increasing or generating one or more activities selected from the group consisting of YGR193C-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of yhr006w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of yhr006w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of H/ACA ribonucleoprotein complex subunit 3 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of H/ACA ribonucleoprotein complex subunit 3 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of glycerol-3-phosphate dehydrogenase in an organelle, preferably in plastids or mitochondria, especially in plastids, of a non-human organism or a part thereof, preferably a microor ganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of glycerol-3-phosphate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of homoserine dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating one or more activities selected from the group consisting of branched chain aminotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of valine, which comprises
(a) increasing or generating one or more activities selected from the group consisting of branched chain aminotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of polygalacturonase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of polygalacturonase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of polygalacturonase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a) increasing or generating one or more activities selected from the group consisting of YKL038W-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of YKL038W-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of YLR224W-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating one or more activities selected from the group consisting of mitochondrial dicarboxylate transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating one or more activities selected from the group consisting of adenylosuccinate lyase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of adenylosuccinate lyase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of adenylosuccinate lyase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of leucine, which comprises
(a) increasing or generating one or more activities selected from the group consisting of phosphate transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of acetyl-CoA acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of acetyl-CoA acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of isoleucine, which comprises
(a) increasing or generating one or more activities selected from the group consisting of cyclin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of isoleucine, which comprises
(a) increasing or generating one or more activities selected from the group consisting of Zm_{—}4842_BE510522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

A further embodiment of the present invention relates to a process for the production of leucine, which comprises
(a) increasing or generating one or more activities selected from the group consisting of Zm_{—}4842_BE510522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

A further embodiment of the present invention relates to a process for the production of valine, which comprises
(a) increasing or generating one or more activities selected from the group consisting of Zm_{—}4842_BE510522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

A further embodiment of the present invention relates to a process for the production of isoleucine, which comprises
(a1) increasing or generating the activity of a polypeptide comprising a polypeptide as depicted in the respective line in column 5 or 8 of Table II, application no. 6, or a homolog or fragment thereof, a consensus sequence or at least one polypeptide motif as depicted in column 8 of Table IV, application no. 6, whereby the respective line discloses in column 7 the fine chemical isoleucine; or
increasing or generating the activity of an expression product of one or more nucleic acid molecule(s) comprising a polynucleotide as depicted in the respective line in column 5 or 8 of Table I, application no. 6, preferably the coding region thereof, or a homolog or a fragment thereof, whereby the respective line discloses in column 7 the fine chemical isoleucine;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 6, or a homolog or a fragment thereof, a consensus sequence or at least one polypeptide motif as depicted in the respective line in column 8 of Table IV, application no. 6, which is joined to a transit peptide, whereby the respective line discloses in column 7 the fine chemical isoleucine; or
increasing or generating the activity of an expression product of one or more nucleic acid molecule(s) comprising a polynucleotide as depicted in the respective line in column 5 or 8 of Table I, application no. 6, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line discloses in column 7 the fine chemical isoleucine;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 6, or a homolog or a fragment thereof, a consensus sequence or at least one polypeptide motif as depicted in the respective line in column 8 of Table IV, application no. 6, whereby the respective line discloses in column 7 the fine chemical isoleucine; or
increasing or generating the activity of an expression product of one or more nucleic acid molecule(s) comprising a polynucleotide as depicted in the respective line in column 5 or 8 of Table I, application no. 6, preferably the coding region thereof, or a homolog or a fragment thereof, whereby the respective line discloses in column 7 the fine chemical isoleucine;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of isoleucine, or a composition comprising isoleucine in said non-human organism or in the culture medium surrounding said non-human organism.

A further embodiment of the present invention relates to a process for the production of leucine, which comprises
(a1) increasing or generating the activity of a polypeptide comprising a polypeptide as depicted in the respective line in column 5 or 8 of Table II, application no. 6, or a homolog or fragment thereof, a consensus sequence or at least one polypeptide motif as depicted in column 8 of Table IV, application no. 6, whereby the respective line discloses in column 7 the fine chemical leucine; or
increasing or generating the activity of an expression product of one or more nucleic acid molecule(s) comprising a polynucleotide as depicted in the respective line in column 5 or 8 of Table I, application no. 6, preferably the coding region thereof, or a homolog or a fragment thereof, whereby the respective line discloses in column 7 the fine chemical leucine;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 6, or a homolog or a fragment thereof, a consensus sequence or at least one polypeptide motif as depicted in the respective line in column 8 of Table IV, application no. 6, which is joined to a transit peptide, whereby the respective line discloses in column 7 the fine chemical leucine; or
increasing or generating the activity of an expression product of one or more nucleic acid molecule(s) comprising a polynucleotide as depicted in the respective line in column 5 or 8 of Table I, application no. 6, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line discloses in column 7 the fine chemical leucine;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 6, or a homolog or a fragment thereof, a consensus sequence or at least one polypeptide motif as depicted in the respective line in column 8 of Table IV, application no. 6, whereby the respective line discloses in column 7 the fine chemical leucine; or
increasing or generating the activity of an expression product of one or more nucleic acid molecule(s) comprising a polynucleotide as depicted in the respective line in column 5 or 8 of Table I, application no. 6, preferably the coding region thereof, or a homolog or a fragment thereof, whereby the respective line discloses in column 7 the fine chemical leucine;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of leucine, or a composition comprising leucine in said non-human organism or in the culture medium surrounding said non-human organism.

A further embodiment of the present invention relates to a process for the production of valine, which comprises
(a1) increasing or generating the activity of a polypeptide comprising a polypeptide as depicted in the respective line in column 5 or 8 of Table II, application no. 6, or a homolog or fragment thereof, a consensus sequence or at least one polypeptide motif as depicted in column 8 of Table IV, application no. 6, whereby the respective line discloses in column 7 the fine chemical valine; or
increasing or generating the activity of an expression product of one or more nucleic acid molecule(s) comprising a polynucleotide as depicted in the respective line in column 5 or 8 of Table I, application no. 6, preferably the coding region thereof, or a homolog or a fragment thereof, whereby the respective line discloses in column 7 the fine chemical valine;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 6, or a homolog or a fragment thereof, a consensus sequence or at least one polypeptide motif as depicted in the respective line in column 8 of Table IV, application no. 6, which is joined to a transit peptide, whereby the respective line discloses in column 7 the fine chemical valine; or
increasing or generating the activity of an expression product of one or more nucleic acid molecule(s) comprising a polynucleotide as depicted in the respective line in column 5 or 8 of Table I, application no. 6, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line discloses in column 7 the fine chemical valine;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 6, or a homolog or a fragment thereof, a consensus sequence or at least one polypeptide motif as depicted in the respective line in column 8 of Table IV, application no. 6, whereby the respective line discloses in column 7 the fine chemical valine; or
increasing or generating the activity of an expression product of one or more nucleic acid molecule(s) comprising a polynucleotide as depicted in the respective line in column 5 or 8 of Table I, application no. 6, preferably the coding region thereof, or a homolog or a fragment thereof, whereby the respective line discloses in column 7 the fine chemical valine;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of valine, or a composition comprising valine in said non-human organism or in the culture medium surrounding said non-human organism.

Accordingly, the present invention relates to a process for the production of isoleucine, which comprises
(a) increasing or generating the activity of a 47266012-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a glucose-6-phosphate 1-dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a glucose-6-phosphate 1-dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a glucose-6-phosphate 1-dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a DNA-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a At1g19800-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a At1g19800-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a At1g23110-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a cullin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a cullin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a eukaryotic translation initiation factor 5 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a eukaryotic translation initiation factor 5 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating the activity of a At1g47380-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a At1g47380-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a CBL-interacting protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a CBL-interacting protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a CBL-interacting protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a thioredoxin family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a thioredoxin family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a harpin-induced family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a harpin-induced family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a DNA mismatch repair protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a DNA mismatch repair protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a DNA mismatch repair protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a eukaryotic translation initiation factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a eukaryotic translation initiation factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a eukaryotic translation initiation factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a glycosyl transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a glycosyl transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a glycosyl transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a auxin response factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a auxin response factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a auxin response factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a At4g32480-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a At4g32480-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a At4g32480-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating the activity of a At5g16650-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating the activity of a At5g16650-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating the activity of a thioredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a thioredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a disease resistance protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a RNA-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a MADS-box transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a MADS-box transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a zinc finger protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a malic enzyme in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a malic enzyme in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a malic enzyme in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a beta-hydroxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a elongation factor Tu in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a elongation factor Tu in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a elongation factor Tu in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a 50S ribosomal protein L14 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a enoyl-CoA hydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a enoyl-CoA hydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a enoyl-CoA hydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a acyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a acyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a acyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a ABC transporter component in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a glucose-1-phosphate cytidylyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a aminotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating the activity of a aminotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a NADH-quinone oxidoreductase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a NADH-quinone oxidoreductase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a glutamate-ammonia-ligase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a glutamate-ammonia-ligase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a glutamate-ammonia-ligase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a isopropylmalate isomerase large subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a fumarylacetoacetate hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a fumarylacetoacetate hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a fumarylacetoacetate hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating the activity of a peptidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a chaperone protein CIpB in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating the activity of a purine nucleoside phosphorylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a purine nucleoside phosphorylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a purine nucleoside phosphorylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a threonine synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a threonine synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a threonine synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a b0050-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating the activity of a acetolactate synthase small subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of leucine, which comprises
(a) increasing or generating the activity of a beta-galactosidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a 2,3-dihydroxyphenylpropionate 1,2-dioxygenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating the activity of a 2,3-dihydroxyphenylpropionate 1,2-dioxygenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a ATP-binding component of a transport system in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a ATP-binding component of a transport system in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a ATP-binding component of a transport system in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a membrane transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating the activity of a isochorismate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a isochorismate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a isochorismate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a zinc transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a zinc transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a major facilitator superfamily transporter protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a major facilitator superfamily transporter protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a major facilitator superfamily transporter protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating the activity of a DNA helicase IV in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a methylglyoxal synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a phosphoanhydride phosphorylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a b1003-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a b1003-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a b1003-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a b1163-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a b1163-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a b1163-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating the activity of a DNA polymerase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a DNA polymerase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a membrane protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a membrane protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a membrane protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a anthranilate synthase component II in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a anthranilate synthase component II in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a anthranilate synthase component II in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating the activity of a glutamine synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a aldehyde dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a b1445-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a NADP-dependent malic enzyme in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a b1522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a b1522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a b1522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a acid shock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a acid shock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a acid shock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a b1670-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a b1672-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a cytochrome c-type protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a trehalose-phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a trehalose-phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a trehalose-phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a O-antigen chain length determinant in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a O-antigen chain length determinant in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a GDP-mannose 4,6-dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a Nucleoside monophosphate phosphohydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a b2513-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a b2513-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a b2513-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a serine hydroxymethyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating the activity of a b2613-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a b2673-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating the activity of a murein transglycosylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a murein transglycosylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a murein transglycosylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a b2739-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a b2739-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a b2739-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a b2812-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a b2812-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a ribosephosphate isomerase, constitutive in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating the activity of a arginine exporter protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a glycoprotease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a glycoprotease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a glycoprotease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a b3083-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating the activity of a b3083-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating the activity of a L-serine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a threonine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating the activity of a acetyl CoA carboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a ABC transporter ATP-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a glycogen synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a glycogen synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a b3490-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a valine-pyruvate transaminase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a valine-pyruvate transaminase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a phosphopantetheine adenylyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a phosphopantetheine adenylyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a phosphopantetheine adenylyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating the activity of a acetolactate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a branched-chain amino-acid aminotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating the activity of a dihydroxyacid dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a dihydroxyacid dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of leucine, which comprises
(a) increasing or generating the activity of a dihydroxyacid dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a dihydroxyacid dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a ketol-acid reductoisomerase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a ketol-acid reductoisomerase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a DNA helicase II in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a DNA helicase II in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a DNA helicase II in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating the activity of a protoporphyrin oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of leucine, which comprises
(a) increasing or generating the activity of a superoxide dismutase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a b3989-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a b3989-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a b3989-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a b4029-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a b4029-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a b4029-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a b4050-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating the activity of a b4056-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a b4056-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a b4263-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a gluconate transport system permease 3 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a gluconate transport system permease 3 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a gluconate transport system permease 3 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a purine-nucleoside phosphorylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating the activity of a thioredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a thioredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a thioredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a GM02LC11114-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a GM02LC11114-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a histone H2A in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a histone H2A in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a histone H2A in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a vacuolar ATPase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a RNA binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a RNA binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a RNA binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating the activity of a GM02LC5744-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a agmatinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a ABC transporter ATP binding component in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a flavodoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a flavodoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a polyphosphate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a polyphosphate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a polyphosphate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a bifunctional protein (phosphoribosyltransferase and regulatory protein) in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a urease subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating the activity of a NAD(P)H-quinone oxidoreductase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a oxireductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating the activity of a binding-protein-dependent transport systems inner membrane component in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating the activity of a sll1032-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a glycogen (starch) synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a glycogen (starch) synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a glycogen (starch) synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a sll1455-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a carbamoyl-phosphate synthase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a carbamoyl-phosphate synthase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a sll1761-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a sll1761-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a adenylate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a adenylate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a adenylate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a acyl transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating the activity of a ferredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a precorrin methylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a precorrin methylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a precorrin methylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a malate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating the activity of a malate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a malate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of leucine, which comprises
(a) increasing or generating the activity of a malate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a malate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a malate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a geranylgeranyl pyrophosphate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a geranylgeranyl pyrophosphate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a geranylgeranyl pyrophosphate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a iron(III) dicitrate-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating the activity of a Na+/K+ transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a Na+/K+ transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a 3-isopropylmalate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a photosystem II protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a photosystem II protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a photosystem II protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a phosphoadenosine phosphosulfate reductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a riboflavin biosynthesis protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a threonine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating the activity of a threonine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a threonine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of leucine, which comprises
(a) increasing or generating the activity of a threonine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a threonine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a threonine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a short-chain alcohol dehydrogenase family in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a Sec-independent protein translocase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a Sec-independent protein translocase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a Sec-independent protein translocase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a 2-isopropylmalate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a 2-isopropylmalate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a TTC1386-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a TTC1386-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a TTC1386-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a homocitrate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a homocitrate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a homocitrate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating the activity of a YAL013W-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a cell division control protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a phosphoglycerate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a phosphoglycerate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a branched-chain amino acid permease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating the activity of a branched-chain amino acid permease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of valine, which comprises
(a) increasing or generating the activity of a ydr183w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a hexose transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating the activity of a ygl237c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a ygl237c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of leucine, which comprises
(a) increasing or generating the activity of a YGR193C-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a yhr006w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a yhr006w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a H/ACA ribonucleoprotein complex subunit 3 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a H/ACA ribonucleoprotein complex subunit 3 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a glycerol-3-phosphate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a glycerol-3-phosphate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a homoserine dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating the activity of a branched chain aminotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of valine, which comprises
(a) increasing or generating the activity of a branched chain aminotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a polygalacturonase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a polygalacturonase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a polygalacturonase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a YKL038W-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a YKL038W-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a YLR224W-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating the activity of a mitochondrial dicarboxylate transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating the activity of a adenylosuccinate lyase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a adenylosuccinate lyase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a adenylosuccinate lyase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of leucine, which comprises
(a) increasing or generating the activity of a phosphate transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a acetyl-CoA acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a acetyl-CoA acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of isoleucine, which comprises
(a) increasing or generating the activity of a cyclin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of isoleucine, which comprises
(a) increasing or generating the activity of a Zm_{—}4842_BE510522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of isoleucine or a composition comprising isoleucine in said non-human organism or in the culture medium surrounding said non-human organism.

Accordingly, the present invention relates to a process for the production of leucine, which comprises
(a) increasing or generating the activity of a Zm_{—}4842_BE510522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of leucine or a composition comprising leucine in said non-human organism or in the culture medium surrounding said non-human organism.

Accordingly, the present invention relates to a process for the production of valine, which comprises
(a) increasing or generating the activity of a Zm_{—}4842_BE510522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of valine or a composition comprising valine in said non-human organism or in the culture medium surrounding said non-human organism.

Accordingly, the present invention relates to a process for the production of isoleucine, which comprises
(a) increasing or generating the activity of a polypeptide comprising a polypeptide as depicted in the respective line in column 5 or 8 of Table II, application no. 6, or a homolog or a fragment thereof, a consensus sequence or at least one polypeptide motif as depicted in the respective line in column 8 of Table IV, application no. 6, whereby the respective line discloses in column 7 the fine chemical isoleucine; or
increasing or generating the activity of an expression product of one or more nucleic acid molecule(s) comprising a polynucleotide as depicted in the respective line in column 5 or 8 of Table I, application no. 6, preferably the coding region thereof, or a homolog or a fragment thereof, whereby the respective line discloses in column 7 the fine chemical isoleucine;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of isoleucine, or a composition comprising isoleucine in said non-human organism or in the culture medium surrounding said non-human organism.

Accordingly, the present invention relates to a process for the production of leucine, which comprises
(a) increasing or generating the activity of a polypeptide comprising a polypeptide as depicted in the respective line in column 5 or 8 of Table II, application no. 6, or a homolog or a fragment thereof, a consensus sequence or at least one polypeptide motif as depicted in the respective line in column 8 of Table IV, application no. 6, whereby the respective line discloses in column 7 the fine chemical leucine; or
increasing or generating the activity of an expression product of one or more nucleic acid molecule(s) comprising a polynucleotide as depicted in the respective line in column 5 or 8 of Table I, application no. 6, preferably the coding region thereof, or a homolog or a fragment thereof, whereby the respective line discloses in column 7 the fine chemical leucine;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of leucine, or a composition comprising leucine in said non-human organism or in the culture medium surrounding said non-human organism.

Accordingly, the present invention relates to a process for the production of valine, which comprises
(a) increasing or generating the activity of a polypeptide comprising a polypeptide as depicted in the respective line in column 5 or 8 of Table II, application no. 6, or a homolog or a fragment thereof, a consensus sequence or at least one polypeptide motif as depicted in the respective line in column 8 of Table IV, application no. 6, whereby the respective line discloses in column 7 the fine chemical valine; or
increasing or generating the activity of an expression product of one or more nucleic acid molecule(s) comprising a polynucleotide as depicted in the respective line in column 5 or 8 of Table I, application no. 6, preferably the coding region thereof, or a homolog or a fragment thereof, whereby the respective line discloses in column 7 the fine chemical valine;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of valine, or a composition comprising valine in said non-human organism or in the culture medium surrounding said non-human organism.

In a further embodiment the activity of the polypeptide comprising a polypeptide as depicted in the respective line in column 5 or 8 of Table II, application no. 6, or a homolog or a fragment thereof, a consensus sequence or at least one polypeptide motif as depicted in the respective line in column 8 of Table IV, application no. 6, is increased or generated non-targeted in the above-mentioned process in a microorganism or plant or a part thereof.

In a further embodiment said polypeptide has the activity of the respective polypeptide represented by a protein comprising a polypeptide as depicted in the respective line in column 5 of Table II, application no. 6.

In a further embodiment the activity of the expression product of one or more nucleic acid molecule(s) comprising a polynucleotide as depicted in the respective line in column 5 or 8 of Table I, application no. 6, preferably the coding region thereof, or a homolog or a fragment thereof, is increased or generated non-targeted in the above-mentioned process in a microorganism or plant or a part thereof.

In a further embodiment the activity of the polypeptide comprising a polypeptide as depicted in the respective line in column 5 or 8 of Table II, application no. 6, or a homolog or a fragment thereof, a consensus sequence or at least one polypeptide motif as depicted in the respective line in column 8 of Table IV, application no. 6, is increased or generated in the above-mentioned process in an organelle preferably in plastids or mitochondria, especially in plastids, of a microorganism or plant.

In a further embodiment said polypeptide has the activity of the respective polypeptide represented by a protein comprising a polypeptide as depicted in the respective line in column 5 of Table II, application no. 6.

In a further embodiment the activity of the expression product of one or more nucleic acid molecule(s) comprising a polynucleotide as depicted in the respective line in column 5 or 8 of Table I, application no. 6, preferably the coding region thereof, or a homolog or a fragment thereof, is increased or generated in the above-mentioned process in an organelle, preferably in plasids or mitochondria, especially in plastids, of a microorganism or plant.

In a further embodiment the activity of the polypeptide comprising a polypeptide as depicted in the respective line in column 5 or 8 of Table II, application no. 6, or a homolog or a fragment thereof, a consensus sequence or at least one polypeptide motif as depicted in the respective line in column 8 of Table IV, application no. 6, is increased or generated in the above-mentioned process in the cyctosol of a cell, of a microorganism or plant.

In a further embodiment said polypeptide has the activity of the respective polypeptide represented by a protein comprising a polypeptide as depicted in the respective line in column 5 of Table II, application no. 6.

In a further embodiment the activity of the expression product of one or more nucleic acid molecule(s) comprising a polynucleotide as depicted in the respective line in column 5 or 8 of Table I, application no. 6, preferably the coding region thereof, or a homolog or a fragment thereof, is increased or generated in the above-mentioned process in the cytosol of a cell, of a microorganism or plant.

[0039.1.1.6] to [0066.1.1.6] for the disclosure of these paragraphs see [0039.1.1.1] to [0066.1.1.1] above.

As mentioned above the nucleic acid sequences coding for the proteins as shown in Table II, application no. 6, column 5 or 8, or homologs or fragments thereof, are joined to a nucleic acid sequence encoding a plastidic transit peptide, e.g, if for the nucleic acid molecule in Table I, column 6 the term “plastidic” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequenceis fused in frame to the nucleic acid sequence coding for proteins as shown in Table II, application no. 6, column 5 or 8, or homologs or fragments thereof, e.g., if for the nucleic acid molecule in Table I, column 6 the term “plastidic” is indicated.

[0068.1.1.6] to [0072.1.1.6] for the disclosure of these paragraphs see [0068.1.1.1] to [0072.1.1.1] above.

As mentioned above the nucleic acid sequences coding for the proteins as shown in the respective line in Table II, application no. 6, column 5 or 8, or homologs or fragments thereof, are joined to a nucleic acid sequence encoding a mitochondric transit peptide, e.g., if for the respective nucleic acid molecule in Table I, column 6 the term “mitochondrial” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in the respective line in Table II, application no. 6, column 5 or 8, or homologs or fragments thereof, e.g., if for the nucleic acid molecule in the respective line in Table I, column 6 the term “mitochondrial” is indicated. (For the avoidance of doubt, the term “mitochondric” and “mitochondrial” are interchangeable.)

[0074.1.1.6] to [0075.1.1.6] for the disclosure of these paragraphs see [0074.1.1.1] to [0075.1.1.1] above. [0076.1.6.6] Nucleic acid sequences coding for the transit peptides may be chemically synthesized either in part or wholly according to structure of transit peptide sequences disclosed in the prior art. Said natural or chemically synthesized sequences can be directly linked to the sequences encoding the mature protein or via a linker nucleic acid sequence, which may be typically less than 500 base pairs, preferably less than 450, 400, 350, 300, 250 or 200 base pairs, more preferably less than 150, 100, 90, 80, 70, 60, 50, 40 or 30 base pairs and most preferably less than 25, 20, 15, 12, 9, 6 or 3 base pairs in length and are in frame to the coding sequence.

Furthermore favorable nucleic acid sequences encoding transit peptides may comprise sequences derived from more than one biological and/or chemical source and may include a nucleic acid sequence derived from the amino-terminal region of the mature protein, which in its native state is linked to the transit peptide. In a preferred embodiment of the invention said amino-terminal region of the mature protein is typically less than 150 amino acids, preferably less than 140, 130, 120, 110, 100 or 90 amino acids, more preferably less than 80, 70, 60, 50, 40, 35, 30, 25 or 20 amino acids and most preferably less than 19, 18, 17, 16, 15, 14, 13, 12, 11 or 10 amino acids in length. But even shorter or longer stretches are also possible. In addition target sequences, which facilitate the transport of proteins to other cell compartments such as the vacuole, endoplasmic reticulum, golgi complex, glyoxysomes, peroxisomes, mitochondria (in case of a plastidal transit peptide) or plastids, especially chloroplasts (in case of a mitochondric transit peptide) may be also part of the inventive nucleic acid sequence. The proteins translated from said inventive nucleic acid sequences are a kind of fusion proteins that means the nucleic acid sequences encoding the plastidal transit peptide for example the ones shown in Table a, preferably the last one of the Table a are joint to the respective nucleic acid sequences shown in Table I, application no. 6, columns 5 or 8, especially the coding region thereof, or homologs or fragments thereof, in case of nucleic acid sequences being expressed in plastids, and that means the nucleic acid sequences encoding the mitochondric transit peptide for example the ones shown in Table b, preferably the last one of the Table b are joint to the respective nucleic acid sequences shown in Table I, application no. 6, columns 5 or 8, especially the coding region thereof, or homologs or fragments thereof, in case of nucleic acid sequences being expressed in mitochondria. The person skilled in the art is able to join said sequences in a functional manner. Advantageously the transit peptide part is cleaved off from the protein part shown in Table II, application no. 6, columns 5 or 8, or a homolog or fragment thereof, during or after the transport into the organelles, preferably into the plastids or mitochondria, respectively.

All products of the cleavage of the preferred transit peptide shown in the last line of Table a have preferably the N-terminal amino acid sequences QIA CSS or QIA EFQLTT in front of the start methionine of the respective protein mentioned in Table II, application no. 6, column 5 or 8. or homologs or fragments thereof. Other short amino acid sequences of an range of 1 to 20 amino acids preferable 2 to 15 amino acids, more preferable 3 to 10 amino acids most preferably 4 to 8 amino acids are also possible in front of the start methionine of the protein mentioned in Table II, application no. 6, columns 5 or 8, or homologs or fragments thereof. In case of the amino acid sequence QIA CSS the three amino acids in front of the start methionine are stemming from the LIC (=ligatation independent cloning) cassette. Said short amino acid sequence is preferred in the case of the expression of E. coli genes. In case of the amino acid sequence QIA EFQLTT the six amino acids in front of the start methionine are stemming from the LIC cassette. Said short amino acid sequence is preferred in the case of the expression of S. cerevisiae genes. The skilled worker knowns that other short sequences are also useful in the expression of the respective nucleic acid molecule comprising a polynucleotide as depicted in Table I, application no. 6, column 5 or 8, preferably the coding region thereof, or a homolog or a fragment thereof. Furthermore the skilled worker is aware of the fact that there is not a need for such short sequences in the expression of the genes.

[0077.1.1.6] to [0078.1.1.6] for the disclosure of these paragraphs see [0077.1.1.1] to see [0078.1.1.1] above.

Alternatively to the targeting of the respective sequences shown in Table II, application no. 6, columns 5 or 8, or homologs or fragments thereof, preferably of sequences in general encoded in the nucleus with the aid of the targeting sequences mentioned for example in Table a alone, Table b alone, or in combination with other targeting sequences preferably into plastids or mitochondria, respectively, the nucleic acids of the invention can directly be introduced into the plastidal or mitochondrial genome. Therefore in a preferred embodiment the respective nucleic acid sequences shown in the respective line in Table I, application no. 6, columns 5 or 8, preferably the coding region thereof, or homologs or fragments thereof, are directly introduced and expressed in plastids e.g., if for the nucleic acid molecule in the respective line in Table I, column 6, the term “plastidic” is indicated, or are directly introduced and expressed in mitochondria e.g., if for the nucleic acid molecule in the respective line in Table I, column 6 the term “mitochondrial” is indicated.

The term “introduced” in the context of this specification shall mean the insertion of a nucleic acid sequence into the organism by means of a “transfection”, “transduction” or preferably by “transformation”.

A plastid, such as a chloroplast, or a mitochondrion, has been “transformed” by an exogenous (preferably foreign) nucleic acid sequence if a nucleic acid sequence has been introduced into the plastid or mitochondrion that means that this sequence has crossed the membrane or the membranes of the plastid or of the mitochondrion. The exogenous, preferably foreign, DNA may be integrated (covalently linked) into plastid or mitochondrial DNA making up the genome of the plastid or mitochondrion, or it may remain unintegrated (e.g., by including a chloroplast origin of replication). “Stably” integrated DNA sequences are those, which are inherited through plastid or mitochondrion replication, thereby transferring new plastids or mitochondria, with the features of the integrated DNA sequence to the progeny.

[0080.1.1.6] to [0083.1.1.6] for the disclosure of these paragraphs see [0080.1.1.1] to [0083.1.1.1] above.

For the process of the present invention it may be of great advantage that by transforming the plastids the intraspecies specific transgene flow is blocked, because a lot of species such as corn, cotton and rice have a strict maternal inheritance of plastids. By placing the respective genes specified in the respective line in Table I, application no. 6, columns 5 or 8, preferably the coding region, or homologs or fragments thereof, e.g. if for the nucleic acid molecule in the respective line in column 6 of Table I the term “plastidic” is indicated, in the plastids of plants, these genes will not be present in the pollen of said plants.

A further preferred embodiment of the invention relates to the use of so called “chloroplast localization sequences”, in which a first RNA sequence or molecule is capable of transporting or “chaperoning” a second RNA sequence, such as a RNA sequence transcribed from the sequences depicted in the respective line in Table I, application no. 6, columns 5 or 8, preferably the coding region thereof, or a homolog or a fragment thereof, or a sequence encoding a protein, as depicted in the respective line in Table II, application no. 6, columns 5 and 8, or a homolog or a fragment thereof, from an external environment inside a cell or outside a plastid into a chloroplast. In one embodiment the chloroplast localization signal is substantially similar or complementary to a complete or intact viroid sequence. The chloroplast localization signal may be encoded by a DNA sequence, which is transcribed into the chloroplast localization RNA. The term “viroid” refers to a naturally occurring single stranded RNA molecule (Flores, C. R. Acad. Sci. III. 324 (10), 943 (2001)). Viroids usually contain about 200-500 nucleotides and generally exist as circular molecules. Examples of viroids that contain chloroplast localization signals include but are not limited to ASBVd, PLMVd, CChMVd and ELVd. The viroid sequence or a functional part of it can be fused to the sequences depicted in the respective line in Table I, application no. 6, columns 5 or 8, preferably the coding region thereof, or a homolog or a fragment thereof, or a sequence encoding a protein, as depicted in the respective line in Table II, application no. 6, columns 5 or 8, or a homolog or a fragment thereof, in such a manner that the viroid sequence transports a sequence transcribed from a sequence as depicted in the respective line in Table I, application no. 6 columns 5 or 8, preferably the coding region thereof, or a homolog or a fragment thereof, or a sequence encoding a protein as depicted in the respective line in Table II, application no. 6 columns 5 or 8, or a homolog or a fragment thereof, into the chloroplasts, e.g. if for the nucleic acid molecule in column 6 of Table I the term “plastidic” is indicated. A preferred embodiment uses a modified ASBVd (Navarro et al., Virology. 268 (1), 218 (2000)).

In a further specific embodiment the protein to be expressed in the plastids such as the proteins depicted in the respective line in Table II, application no. 6, columns 5 or 8, or a homolog or a fragment thereof, e.g. if for the nucleic acid molecule in the respective line in column 6 of Table I the term “plastidic” is indicated, are encoded by different nucleic acids. Such a method is disclosed in WO 2004/040973, which shall be incorporated by reference. WO 2004/040973 teaches a method, which relates to the translocation of an RNA corresponding to a gene or gene fragment into the chloroplast by means of a chloroplast localization sequence. The genes, which should be expressed in the plant or plant cells, are split into nucleic acid fragments, which are introduced into different compartments in the plant e.g. the nucleus, the plastids and/or mitochondria. Additionally plant cells are described in which the chloroplast contains a ribozyme fused at one end to an RNA encoding a fragment of a protein used in the inventive process such that the ribozyme can trans-splice the translocated fusion RNA to the RNA encoding the gene fragment to form and as the case may be reunite the nucleic acid fragments to an intact mRNA encoding a functional protein for example as disclosed in the respective line in Table II, application no. 6, columns 5 or 8, or a homolog or a fragment thereof.

In a preferred embodiment of the invention the nucleic acid sequences as shown in the respective line in Table I, application no. 6, columns 5 or 8, preferably the coding region thereof, or homologs or fragments thereof, e.g. if for the nucleic acid molecule in the respective line in column 6 of Table I the term “plastidic” is indicated, used in the inventive process are transformed into plastids, which are metabolically active. Those plastids should preferably maintain at a high copy number in the plant or plant tissue of interest, most preferably the chloroplasts found in green plant tissues, such as leaves or cotyledons or in seeds.

For a good expression in the plastids the nucleic acid sequences as shown in the respective line in Table I, application no. 6, columns 5 or 8, preferably the coding region thereof, or homologs or fragments thereof, e.g. if for the nucleic acid molecule in the respective line in column 6 of Table I the term “plastidic” is indicated, are introduced into an expression cassette using preferably a promoter and terminater, which are active in plastids preferably a chloroplast promoter. Examples of such promoters include the psbA promoter from the gene from spinach or pea, the rbcL promoter, and the atpB promoter from corn.

[0089.1.1.6] to [0092.1.1.6] for the disclosure of these paragraphs see [0089.1.1.1] to [0092.1.1.1] above.

Advantageously the process for the production of the fine chemical isoleucine, leucine, and/or valine leads to an enhanced production of the respective fine chemical. The terms “enhanced” or “increase” mean at least a 10%, 20%, 30%, 40% or 50%, preferably at least 60%, 70%, 80%, 90% or 100%, more preferably 150%, 200%, 300%, 400% or 500% higher production of the respective fine chemical isoleucine, leucine, and/or valine in comparison to the wild-type as defined above, e.g. that means in comparison to a non-human organism without the aforementioned modification of the activity of a protein as shown in the respective line in Table II, application no. 6, column 5 or 8, or a fragment or a homolog thereof. The modification of the activity of a protein as shown in the respective line in Table II, application no. 6, column 5 or 8, or a homolog or a fragment thereof, or their combination can be achieved by joining the protein to a respective transit peptide, e.g. if for the respective encoding nucleic acid molecule in column 6 of Table I the term “plastidic” or “mitochondrial” is indicated.

Surprisingly it was found, that the transgenic expression of a protein as shown in the respective line in Table II, application no. 6, column 5, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, conferred a production of or an increase in isoleucine, leucine, and/or valine,respectively, to the transgenic non-human organism as compared to a corresponding non-transformed wild type.

In one embodiment the transgenic expression of a protein as shown in the respective line in Table II, application no. 4, column 5 or 8, or a homolog or a fragment thereof, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, conferred a production of or an increase in threonine,respectively, to the transgenic non-human organism as compared to a corresponding non-transformed wild type.

Accordingly, in one embodiment, an increase of isoleucine in a non-human organism, as compared to a corresponding non-transformed wild type non-human organism, is conferred in the process of the invention, if the activity of a polypeptide showing the activity of a 47266012-protein, or if the activity of the polypeptide 47266012_SOYBEAN, preferably represented by SEQ ID NO. 15188, or a homolog or fragment thereof, or if the activity of a polypeptide encoded by a nucleic acid molecule comprising the nucleic acid SEQ ID NO. 15187, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 15187 or polypeptide SEQ ID NO. 15188, respectively, or a homolog or a fragment thereof, is increased or generated, or if the activity 47266012-protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 36 to 43-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a pyruvate kinase, or if the activity of the polypeptide 51340801_CANOLA, preferably represented by SEQ ID NO. 15533, or a homolog or fragment thereof, or if the activity of a polypeptide encoded by a nucleic acid molecule comprising the nucleic acid SEQ ID NO. 15532, preferably the coding region thereof, or a homolog or fragment thereof, e.g. derived from Brassica napus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 15532 or polypeptide SEQ ID NO. 15533, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 80 to 500-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a pyruvate kinase, or if the activity of the polypeptide 51340801_CANOLA, preferably represented by SEQ ID NO. 15533, or a homolog or fragment thereof, or if the activity of a polypeptide encoded by a nucleic acid molecule comprising the nucleic acid SEQ ID NO. 15532, preferably the coding region thereof, or a homolog or fragment thereof, e.g. derived from Brassica napus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 15532 or polypeptide SEQ ID NO. 15533, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line discloses in column 7 the fine chemical valine. For example, an increase of the valine of at least 1 percent, particularly in a range of 34 to 207-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a pyruvate kinase, or if the activity of the polypeptide 59554615_SOYBEAN, preferably represented by SEQ ID NO. 16264, or a homolog or fragment thereof, or if the activity of a polypeptide encoded by a nucleic acid molecule comprising the nucleic acid SEQ ID NO. 16263, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 16263 or polypeptide SEQ ID NO. 16264, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line discloses in column 7 the fine chemical valine. For example, an increase of the valine of at least 1 percent, particularly in a range of 22 to 74-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a glucose-6phosphate 1-dehydrogenase, or if the activity of the polypeptide 59582753_SOYBEAN, preferably represented by SEQ ID NO. 16884, or a homolog or fragment thereof, or if the activity of a polypeptide encoded by a nucleic acid molecule comprising the nucleic acid SEQ ID NO. 16883, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 16883 or polypeptide SEQ ID NO. 16884, respectively, or a homolog or a fragment thereof, is increased or generated, or if the activity glucose-6-phosphate 1-dehydrogenase is increased or generated in a non-human organism, like a microorganism or a plant cell, plant or part thereof, especially with plastidic localization, whereby the respective line discloses in column 7 the fine chemical isoleucine. For example, an increase of the isoleucine of at least 1 percent, particularly in a range of 33 to 45-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a glucose-6-phosphate 1-dehydrogenase, or if the activity of the polypeptide 59582753_SOYBEAN, preferably represented by SEQ ID NO. 16884, or a homolog or fragment thereof, or if the activity of a polypeptide encoded by a nucleic acid molecule comprising the nucleic acid SEQ ID NO. 16883, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 16883 or polypeptide SEQ ID NO. 16884, respectively, or a homolog or a fragment thereof, is increased or generated, or if the activity glucose-6-phosphate 1-dehydrogenase is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 47 to 54-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a glucose-6-phosphate 1-dehydrogenase, or if the activity of the polypeptide 59582753_SOYBEAN, preferably represented by SEQ ID NO. 16884, or a homolog or fragment thereof, or if the activity of a polypeptide encoded by a nucleic acid molecule comprising the nucleic acid SEQ ID NO. 16883, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 16883 or polypeptide SEQ ID NO. 16884, respectively, or a homolog or a fragment thereof, is increased or generated, or if the activity glucose-6-phosphate 1-dehydrogenase is increased or generated in a non-human organism, like a microorganism or a plant cell, plant or part thereof, especially with plastidic localization, whereby the respective line discloses in column 7 the fine chemical valine. For example, an increase of the valine of at least 1 percent, particularly in a range of 45 to 97-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a protein phosphatase, or if the activity of the polypeptide At1 g07430, preferably represented by SEQ ID NO. 17452, or a homolog or fragment thereof, or if the activity of a polypeptide encoded by a nucleic acid molecule comprising the nucleic acid SEQ ID NO. 17451, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 17451 or polypeptide SEQ ID NO. 17452, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 24 to 39-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a DNA-binding protein, or if the activity of the polypeptide At1g14490, preferably represented by SEQ ID NO. 386, or a homolog or fragment thereof, or if the activity of a polypeptide encoded by a nucleic acid molecule comprising the nucleic acid SEQ ID NO. 385, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 385 or polypeptide SEQ ID NO. 386, respectively, or a homolog or a fragment thereof, is increased or generated, or if the activity DNA-binding protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 21 to 54-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a At1g19800-protein, or if the activity of the polypeptide At1g19800, preferably represented by SEQ ID NO. 17638, or a homolog or fragment thereof, or if the activity of a polypeptide encoded by a nucleic acid molecule comprising the nucleic acid SEQ ID NO. 17637, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 17637 or polypeptide SEQ ID NO. 17638, respectively, or a homolog or a fragment thereof, is increased or generated, or if the activity At1g19800-protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 45 to 805-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a At1g19800-protein, or if the activity of the polypeptide At1g19800, preferably represented by SEQ ID NO. 17638, or a homolog or fragment thereof, or if the activity of a polypeptide encoded by a nucleic acid molecule comprising the nucleic acid SEQ ID NO. 17637, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 17637 or polypeptide SEQ ID NO. 17638, respectively, or a homolog or a fragment thereof, is increased or generated, or if the activity At1g19800-protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 115 to 1238-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a At1g23110-protein, or if the activity of the polypeptide At1g23110, preferably represented by SEQ ID NO. 102087, or a homolog or fragment thereof, or if the activity of a polypeptide encoded by a nucleic acid molecule comprising the nucleic acid SEQ ID NO. 102086, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 102086 or polypeptide SEQ ID NO. 102087, respectively, or a homolog or a fragment thereof, is increased or generated, or if the activity At1g23110-protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 28 to 40-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a cullin, or if the activity of the polypeptide At1g26830, preferably represented by SEQ ID NO. 17702, or a homolog or fragment thereof, or if the activity of a polypeptide encoded by a nucleic acid molecule comprising the nucleic acid SEQ ID NO. 17701, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 17701 or polypeptide SEQ ID NO. 17702, respectively, or a homolog or a fragment thereof, is increased or generated, or if the activity cullin is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 45 to 143-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a cullin, or if the activity of the polypeptide At1g26830, preferably represented by SEQ ID NO. 17702, or a homolog or fragment thereof, or if the activity of a polypeptide encoded by a nucleic acid molecule comprising the nucleic acid SEQ ID NO. 17701, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 17701 or polypeptide SEQ ID NO. 17702, respectively, or a homolog or a fragment thereof, is increased or generated, or if the activity cullin is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 59 to 187-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a eukaryotic translation initiation factor 5, or if the activity of the polypeptide At1g36730, preferably represented by SEQ ID NO. 17969, or a homolog or fragment thereof, or if the activity of a polypeptide encoded by a nucleic acid molecule comprising the nucleic acid SEQ ID NO. 17968, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 17968 or polypeptide SEQ ID NO. 17969, respectively, or a homolog or a fragment thereof, is increased or generated, or if the activity eukaryotic translation initiation factor 5 is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 49 to 162-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a eukaryotic translation initiation factor 5, or if the activity of the polypeptide At1g36730, preferably represented by SEQ ID NO. 17969, or a homolog or fragment thereof, or if the activity of a polypeptide encoded by a nucleic acid molecule comprising the nucleic acid SEQ ID NO. 17968, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 17968 or polypeptide SEQ ID NO. 17969, respectively, or a homolog or a fragment thereof, is increased or generated, or if the activity eukaryotic translation initiation factor 5 is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 52 to 149-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a transcriptional regulator, or if the activity of the polypeptide At1g43850, preferably represented by SEQ ID NO. 18071, or a homolog or fragment thereof, or if the activity of a polypeptide encoded by a nucleic acid molecule comprising the nucleic acid SEQ ID NO. 18070, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 18070 or polypeptide SEQ ID NO. 18071, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 69 to 406-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a transcriptional regulator, or if the activity of the polypeptide At1g43850, preferably represented by SEQ ID NO. 18071, or a homolog or fragment thereof, or if the activity of a polypeptide encoded by a nucleic acid molecule comprising the nucleic acid SEQ ID NO. 18070, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 18070 or polypeptide SEQ ID NO. 18071, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 58 to 377-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a transcriptional regulator, or if the activity of the polypeptide At1g43850, preferably represented by SEQ ID NO. 18071, or a homolog or fragment thereof, or if the activity of a polypeptide encoded by a nucleic acid molecule comprising the nucleic acid SEQ ID NO. 18070, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 18070 or polypeptide SEQ ID NO. 18071, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 58 to 173-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a At1g47380-protein, or if the activity of the polypeptide At1g47380, preferably represented by SEQ ID NO. 18123, or a homolog or fragment thereof, or if the activity of a polypeptide encoded by a nucleic acid molecule comprising the nucleic acid SEQ ID NO. 18122, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 18122 or polypeptide SEQ ID NO. 18123, respectively, or a homolog or a fragment thereof, is increased or generated, or if the activity At1g47380-protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 51 to 134-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a At1g47380-protein, or if the activity of the polypeptide At1g47380, preferably represented by SEQ ID NO. 18123, or a homolog or fragment thereof, or if the activity of a polypeptide encoded by a nucleic acid molecule comprising the nucleic acid SEQ ID NO. 18122, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 18122 or polypeptide SEQ ID NO. 18123, respectively, or a homolog or a fragment thereof, is increased or generated, or if the activity At1g47380-protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 20 to 105-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a protein kinase, or if the activity of the polypeptide At1g48260, preferably represented by SEQ ID NO. 18236, or a homolog or fragment thereof, or if the activity of a polypeptide encoded by a nucleic acid molecule comprising the nucleic acid SEQ ID NO. 18235, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 18235 or polypeptide SEQ ID NO. 18236, respectively, or a homolog or a fragment thereof, is increased or generated, or if the activity protein kinase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 78 to 747-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a protein kinase, or if the activity of the polypeptide At1g48260, preferably represented by SEQ ID NO. 18236, or a homolog or fragment thereof, or if the activity of a polypeptide encoded by a nucleic acid molecule comprising the nucleic acid SEQ ID NO. 18235, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 18235 or polypeptide SEQ ID NO. 18236, respectively, or a homolog or a fragment thereof, is increased or generated, or if the activity protein kinase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 86 to 639-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a protein kinase, or if the activity of the polypeptide At1g48260, preferably represented by SEQ ID NO. 18236, or a homolog or fragment thereof, or if the activity of a polypeptide encoded by a nucleic acid molecule comprising the nucleic acid SEQ ID NO. 18235, preferably the coding region thereof, or a homolog or fragment thereof, e.g. derived from Arabidopsis thaliana, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 18235 or polypeptide SEQ ID NO. 18236, respectively, or a homolog or a fragment thereof, is increased or generated, or if the activity protein kinase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 51 to 277-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a calcium-dependent protein kinase, or if the activity of the polypeptide At1g61950, preferably represented by SEQ ID NO. 18870, or a homolog or fragment thereof, or if the activity of a polypeptide encoded by a nucleic acid molecule comprising the nucleic acid SEQ ID NO. 18869, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 18869 or polypeptide SEQ ID NO. 18870, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 96 to 174-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a calcium-dependent protein kinase, or if the activity of the polypeptide At1g61950, preferably represented by SEQ ID NO. 18870, or a homolog or fragment thereof, or if the activity of a polypeptide encoded by a nucleic acid molecule comprising the nucleic acid SEQ ID NO. 18869, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 18869 or polypeptide SEQ ID NO. 18870, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 99 to 169-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a calcium-dependent protein kinase, or if the activity of the polypeptide At1g61950, preferably represented by SEQ ID NO. 18870, or a homolog or fragment thereof, or if the activity of a polypeptide encoded by a nucleic acid molecule comprising the nucleic acid SEQ ID NO. 18869, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 18869 or polypeptide SEQ ID NO. 18870, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 43 to 84-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a protein phosphatase, or if the activity of the polypeptide At1g68410, preferably represented by SEQ ID NO. 102120, or a homolog or fragment thereof, or if the activity of a polypeptide encoded by a nucleic acid molecule comprising the nucleic acid SEQ ID NO. 102119, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 102119 or polypeptide SEQ ID NO. 102120, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 41 to 140-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a protein phosphatase, or if the activity of the polypeptide At1g68410, preferably represented by SEQ ID NO. 102120, or a homolog or fragment thereof, or if the activity of a polypeptide encoded by a nucleic acid molecule comprising the nucleic acid SEQ ID NO. 102119, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 102119 or polypeptide SEQ ID NO. 102120, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 50 to 137-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a protein phosphatase, or if the activity of the polypeptide At1g72770, preferably represented by SEQ ID NO. 19420, or a homolog or fragment thereof, or if the activity of a polypeptide encoded by a nucleic acid molecule comprising the nucleic acid SEQ ID NO. 19419, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 19419 or polypeptide SEQ ID NO. 19420, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 22 to 134-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a transcription factor, or if the activity of the polypeptide At2g17560, preferably represented by SEQ ID NO. 19503, or a homolog or fragment thereof, or if the activity of a polypeptide encoded by a nucleic acid molecule comprising the nucleic acid SEQ ID NO. 19502, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 19502 or polypeptide SEQ ID NO. 19503, respectively, or a homolog or a fragment thereof, is increased or generated, or if the activity transcription factor is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 25 to 108-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a protein phosphatase, or if the activity of the polypeptide At2g28890, preferably represented by SEQ ID NO. 19875, or a homolog or fragment thereof, or if the activity of a polypeptide encoded by a nucleic acid molecule comprising the nucleic acid SEQ ID NO. 19874, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 19874 or polypeptide SEQ ID NO. 19875, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 33 to 738-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a protein phosphatase, or if the activity of the polypeptide At2g28890, preferably represented by SEQ ID NO. 19875, or a homolog or fragment thereof, or if the activity of a polypeptide encoded by a nucleic acid molecule comprising the nucleic acid SEQ ID NO. 19874, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 19874 or polypeptide SEQ ID NO. 19875, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 26 to 321 percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a CBL-interacting protein kinase, or if the activity of the polypeptide At2g30360, preferably represented by SEQ ID NO. 19920, or a homolog or fragment thereof, or if the activity of a polypeptide encoded by a nucleic acid molecule comprising the nucleic acid SEQ ID NO. 19919, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 19919 or polypeptide SEQ ID NO. 19920, respectively, or a homolog or a fragment thereof, is increased or generated, or if the activity CBL-interacting protein kinase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 35 to 810-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a CBL-interacting protein kinase, or if the activity of the polypeptide At2g30360, preferably represented by SEQ ID NO. 19920, or a homolog or fragment thereof, or if the activity of a polypeptide encoded by a nucleic acid molecule comprising the nucleic acid SEQ ID NO. 19919, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 19919 or polypeptide SEQ ID NO. 19920, respectively, or a homolog or a fragment thereof, is increased or generated, or if the activity CBL-interacting protein kinase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 64 to 947-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a CBL-interacting protein kinase, or if the activity of the polypeptide At2g30360, preferably represented by SEQ ID NO. 19920, or a homolog or fragment thereof, or if the activity of a polypeptide encoded by a nucleic acid molecule comprising the nucleic acid SEQ ID NO. 19919, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 19919 or polypeptide SEQ ID NO. 19920, respectively, or a homolog or a fragment thereof, is increased or generated, or if the activity CBL-interacting protein kinase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 137 to 277-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a glutaredoxin, or if the activity of the polypeptide At2g30540, preferably represented by SEQ ID NO. 20347, or a homolog or fragment thereof, or if the activity of a polypeptide encoded by a nucleic acid molecule comprising the nucleic acid SEQ ID NO. 20346, preferably the coding region thereof, or a homolog or fragment thereof, e.g. derived from Arabidopsis thaliana, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 20346 or polypeptide SEQ ID NO. 20347, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 39 to 206-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a glutaredoxin, or if the activity of the polypeptide At2g30540, preferably represented by SEQ ID NO. 20347, or a homolog or fragment thereof, or if the activity of a polypeptide encoded by a nucleic acid molecule comprising the nucleic acid SEQ ID NO. 20346, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 20346 or polypeptide SEQ ID NO. 20347, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 66 to 243-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a glutaredoxin, or if the activity of the polypeptide At2g30540, preferably represented by SEQ ID NO. 20347, or a homolog or fragment thereof, or if the activity of a polypeptide encoded by a nucleic acid molecule comprising the nucleic acid SEQ ID NO. 20346, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 20346 or polypeptide SEQ ID NO. 20347, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 24 to 183-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a glutaredoxin, or if the activity of the polypeptide At2g47880, preferably represented by SEQ ID NO. 21160, or a homolog or fragment thereof, or if the activity of a polypeptide encoded by a nucleic acid molecule comprising the nucleic acid SEQ ID NO. 21159, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 21159 or polypeptide SEQ ID NO. 21160, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 65 to 227-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a pyruvate kinase, or if the activity of the polypeptide At3g04050, preferably represented by SEQ ID NO. 21498, or a homolog or fragment thereof, or if the activity of a polypeptide encoded by a nucleic acid molecule comprising the nucleic acid SEQ ID NO. 21497, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 21497 or polypeptide SEQ ID NO. 21498, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line discloses in column 7 the fine chemical isoleucine. For example, an increase of the isoleucine of at least 1 percent, particularly in a range of 39 to 470-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a pyruvate kinase, or if the activity of the polypeptide At3g04050, preferably represented by SEQ ID NO. 21498, or a homolog or fragment thereof, or if the activity of a polypeptide encoded by a nucleic acid molecule comprising the nucleic acid SEQ ID NO. 21497, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 21497 or polypeptide SEQ ID NO. 21498, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 64 to 1234-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a pyruvate kinase, or if the activity of the polypeptide At3g04050, preferably represented by SEQ ID NO. 21498, or a homolog or fragment thereof, or if the activity of a polypeptide encoded by a nucleic acid molecule comprising the nucleic acid SEQ ID NO. 21497, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 21497 or polypeptide SEQ ID NO. 21498, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line discloses in column 7 the fine chemical valine. For example, an increase of the valine of at least 1-percent, particularly in a range of 35 to 681-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a thioredoxin family protein, or if the activity of the polypeptide At3g08710, preferably represented by SEQ ID NO. 22250, or a homolog or fragment thereof, or if the activity of a polypeptide encoded by a nucleic acid molecule comprising the nucleic acid SEQ ID NO. 22249, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 22249 or polypeptide SEQ ID NO. 22250, respectively, or a homolog or a fragment thereof, is increased or generated, or if the activity thioredoxin family protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 34 to 264-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a thioredoxin family protein, or if the activity of the polypeptide At3g08710, preferably represented by SEQ ID NO. 22250, or a homolog or fragment thereof, or if the activity of a polypeptide encoded by a nucleic acid molecule comprising the nucleic acid SEQ ID NO. 22249, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 22249 or polypeptide SEQ ID NO. 22250, respectively, or a homolog or a fragment thereof, is increased or generated, or if the activity thioredoxin family protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 53 to 221-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a harpin-induced family protein, or if the activity of the polypeptide At3g11650, preferably represented by SEQ ID NO. 22612, or a homolog or fragment thereof, or if the activity of a polypeptide encoded by a nucleic acid molecule comprising the nucleic acid SEQ ID NO. 22611, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 22611 or polypeptide SEQ ID NO. 22612, respectively, or a homolog or a fragment thereof, is increased or generated, or if the activity harpin-induced family protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 50 to 191-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a harpin-induced family protein, or if the activity of the polypeptide At3g11650, preferably represented by SEQ ID NO. 22612, or a homolog or fragment thereof, or if the activity of a polypeptide encoded by a nucleic acid molecule comprising the nucleic acid SEQ ID NO. 22611, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 22611 or polypeptide SEQ ID NO. 22612, respectively, or a homolog or a fragment thereof, is increased or generated, or if the activity harpin-induced family protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 52 to 216-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a DNA mismatch repair protein, or if the activity of the polypeptide At3g18524, preferably represented by SEQ ID NO. 22833, or a homolog or fragment thereof, or if the activity of a polypeptide encoded by a nucleic acid molecule comprising the nucleic acid SEQ ID NO. 22832, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 22832 or polypeptide SEQ ID NO. 22833, respectively, or a homolog or a fragment thereof, is increased or generated, or if the activity DNA mismatch repair protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 34 to 61-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a DNA mismatch repair protein, or if the activity of the polypeptide At3g18524, preferably represented by SEQ ID NO. 22833, or a homolog or fragment thereof, or if the activity of a polypeptide encoded by a nucleic acid molecule comprising the nucleic acid SEQ ID NO. 22832, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 22832 or polypeptide SEQ ID NO. 22833, respectively, or a homolog or a fragment thereof, is increased or generated, or if the activity DNA mismatch repair protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 61 to 87-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a DNA mismatch repair protein, or if the activity of the polypeptide At3g18524, preferably represented by SEQ ID NO. 22833, or a homolog or fragment thereof, or if the activity of a polypeptide encoded by a nucleic acid molecule comprising the nucleic acid SEQ ID NO. 22832, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 22832 or polypeptide SEQ ID NO. 22833, respectively, or a homolog or a fragment thereof, is increased or generated, or if the activity DNA mismatch repair protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 21 to 31-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a protein kinase, or if the activity of the polypeptide At3g23000, preferably represented by SEQ ID NO. 1816, or a homolog or fragment thereof, or if the activity of a polypeptide encoded by a nucleic acid molecule comprising the nucleic acid SEQ ID NO. 1815, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 1815 or polypeptide SEQ ID NO. 1816, respectively, or a homolog or a fragment thereof, is increased or generated, or if the activity protein kinase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 63 to 717-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a protein kinase, or if the activity of the polypeptide At3g23000, preferably represented by SEQ ID NO. 1816, or a homolog or fragment thereof, or if the activity of a polypeptide encoded by a nucleic acid molecule comprising the nucleic acid SEQ ID NO. 1815, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 1815 or polypeptide SEQ ID NO. 1816, respectively, or a homolog or a fragment thereof, is increased or generated, or if the activity protein kinase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 82 to 562-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a protein kinase, or if the activity of the polypeptide At3g23000, preferably represented by SEQ ID NO. 1816, or a homolog or fragment thereof, or if the activity of a polypeptide encoded by a nucleic acid molecule comprising the nucleic acid SEQ ID NO. 1815, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 1815 or polypeptide SEQ ID NO. 1816, respectively, or a homolog or a fragment thereof, is increased or generated, or if the activity protein kinase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 42 to 249-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a eukaryotic translation initiation factor, or if the activity of the polypeptide At3g26400, preferably represented by SEQ ID NO. 68728, or a homolog or fragment thereof, or if the activity of a polypeptide encoded by a nucleic acid molecule comprising the nucleic acid SEQ ID NO. 68727, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 68727 or polypeptide SEQ ID NO. 68728, respectively, or a homolog or a fragment thereof, is increased or generated, or if the activity eukaryotic translation initiation factor is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 46 to 133-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a eukaryotic translation initiation factor, or if the activity of the polypeptide At3g26400, preferably represented by SEQ ID NO. 68728, or a homolog or fragment thereof, or if the activity of a polypeptide encoded by a nucleic acid molecule comprising the nucleic acid SEQ ID NO. 68727, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 68727 or polypeptide SEQ ID NO. 68728, respectively, or a homolog or a fragment thereof, is increased or generated, or if the activity eukaryotic translation initiation factor is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 51 to 129-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a eukaryotic translation initiation factor, or if the activity of the polypeptide At3g26400, preferably represented by SEQ ID NO. 68728, or a homolog or fragment thereof, or if the activity of a polypeptide encoded by a nucleic acid molecule comprising the nucleic acid SEQ ID NO. 68727, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 68727 or polypeptide SEQ ID NO. 68728, respectively, or a homolog or a fragment thereof, is increased or generated, or if the activity eukaryotic translation initiation factor is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 32 to 76-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a glycosyl transferase, or if the activity of the polypeptide At3g27540, preferably represented by SEQ ID NO. 68778, or a homolog or fragment thereof, or if the activity of a polypeptide encoded by a nucleic acid molecule comprising the nucleic acid SEQ ID NO. 68777, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 68777 or polypeptide SEQ ID NO. 68778, respectively, or a homolog or a fragment thereof, is increased or generated, or if the activity glycosyl transferase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 79 to 428-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a glycosyl transferase, or if the activity of the polypeptide At3g27540, preferably represented by SEQ ID NO. 68778, or a homolog or fragment thereof, or if the activity of a polypeptide encoded by a nucleic acid molecule comprising the nucleic acid SEQ ID NO. 68777, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 68777 or polypeptide SEQ ID NO. 68778, respectively, or a homolog or a fragment thereof, is increased or generated, or if the activity glycosyl transferase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 89 to 469-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a glycosyl transferase, or if the activity of the polypeptide At3g27540, preferably represented by SEQ ID NO. 68778, or a homolog or fragment thereof, or if the activity of a polypeptide encoded by a nucleic acid molecule comprising the nucleic acid SEQ ID NO. 68777, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 68777 or polypeptide SEQ ID NO. 68778, respectively, or a homolog or a fragment thereof, is increased or generated, or if the activity glycosyl transferase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 26 to 161-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a auxin response factor, or if the activity of the polypeptide At3g61830, preferably represented by SEQ ID NO. 2368, or a homolog or fragment thereof, or if the activity of a polypeptide encoded by a nucleic acid molecule comprising the nucleic acid SEQ ID NO. 2367, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 2367 or polypeptide SEQ ID NO. 2368, respectively, or a homolog or a fragment thereof, is increased or generated, or if the activity auxin response factor is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 39 to 965-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a auxin response factor, or if the activity of the polypeptide At3g61830, preferably represented by SEQ ID NO. 2368, or a homolog or fragment thereof, or if the activity of a polypeptide encoded by a nucleic acid molecule comprising the nucleic acid SEQ ID NO. 2367, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 2367 or polypeptide SEQ ID NO. 2368, respectively, or a homolog or a fragment thereof, is increased or generated, or if the activity auxin response factor is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 67 to 1444-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a auxin response factor, or if the activity of the polypeptide At3g61830, preferably represented by SEQ ID NO. 2368, or a homolog or fragment thereof, or if the activity of a polypeptide encoded by a nucleic acid molecule comprising the nucleic acid SEQ ID NO. 2367, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 2367 or polypeptide SEQ ID NO. 2368, respectively, or a homolog or a fragment thereof, is increased or generated, or if the activity auxin response factor is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 27 to 397-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 60 to 274-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 85 to 337-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 37 to 118-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a heat shock transcription factor, or if the activity of the polypeptide At4g18880, preferably represented by SEQ ID NO. 24233, or a homolog or fragment thereof, or if the activity of a polypeptide encoded by a nucleic acid molecule comprising the nucleic acid SEQ ID NO. 24232, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 24232 or polypeptide SEQ ID NO. 24233, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 52 to 136-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a protein phosphatase, or if the activity of the polypeptide At4g26080, preferably represented by SEQ ID NO. 68850, or a homolog or fragment thereof, or if the activity of a polypeptide encoded by a nucleic acid molecule comprising the nucleic acid SEQ ID NO. 68849, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 68849 or polypeptide SEQ ID NO. 68850, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 21 to 56-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a At4g32480-protein, or if the activity of the polypeptide At4g32480, preferably represented by SEQ ID NO. 4041, or a homolog or fragment thereof, or if the activity of a polypeptide encoded by a nucleic acid molecule comprising the nucleic acid SEQ ID NO. 4040, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 4040 or polypeptide SEQ ID NO. 4041, respectively, or a homolog or a fragment thereof, is increased or generated, or if the activity At4g32480-protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 74 to 355-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a At4g32480-protein, or if the activity of the polypeptide At4g32480, preferably represented by SEQ ID NO. 4041, or a homolog or fragment thereof, or if the activity of a polypeptide encoded by a nucleic acid molecule comprising the nucleic acid SEQ ID NO. 4040, preferably the coding region thereof, or a homolog or fragment thereof, e.g. derived from Arabidopsis thaliana, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 4040 or polypeptide SEQ ID NO. 4041, respectively, or a homolog or a fragment thereof, is increased or generated, or if the activity At4g32480-protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 53 to 320-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a At4g32480-protein, or if the activity of the polypeptide At4g32480, preferably represented by SEQ ID NO. 4041, or a homolog or fragment thereof, or if the activity of a polypeptide encoded by a nucleic acid molecule comprising the nucleic acid SEQ ID NO. 4040, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 4040 or polypeptide SEQ ID NO. 4041, respectively, or a homolog or a fragment thereof, is increased or generated, or if the activity At4g32480-protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 29 to 286-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a cyclin D , or if the activity of the polypeptide At4g34160, preferably represented by SEQ ID NO. 24312, or a homolog or fragment thereof, or if the activity of a polypeptide encoded by a nucleic acid molecule comprising the nucleic acid SEQ ID NO. 24311, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 24311 or polypeptide SEQ ID NO. 24312, respectively, or a homolog or a fragment thereof, is increased or generated, or if the activity cyclin D is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 46 to 199-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a cyclin D , or if the activity of the polypeptide At4g34160, preferably represented by SEQ ID NO. 24312, or a homolog or fragment thereof, or if the activity of a polypeptide encoded by a nucleic acid molecule comprising the nucleic acid SEQ ID NO. 24311, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 24311 or polypeptide SEQ ID NO. 24312, respectively, or a homolog or a fragment thereof, is increased or generated, or if the activity cyclin D is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 83 to 188-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a cyclin D , or if the activity of the polypeptide At4g34160, preferably represented by SEQ ID NO. 24312, or a homolog or fragment thereof, or if the activity of a polypeptide encoded by a nucleic acid molecule comprising the nucleic acid SEQ ID NO. 24311, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 24311 or polypeptide SEQ ID NO. 24312, respectively, or a homolog or a fragment thereof, is increased or generated, or if the activity cyclin D is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 23 to 115-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 35 to 645-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 164 to 698-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 39 to 329-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a protein phosphatase, or if the activity of the polypeptide At5g02760, preferably represented by SEQ ID NO. 68988, or a homolog or fragment thereof, or if the activity of a polypeptide encoded by a nucleic acid molecule comprising the nucleic acid SEQ ID NO. 68987, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 68987 or polypeptide SEQ ID NO. 68988, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 40 to 305-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a At5g16650-protein, or if the activity of the polypeptide At5g16650, preferably represented by SEQ ID NO. 25284, or a homolog or fragment thereof, or if the activity of a polypeptide encoded by a nucleic acid molecule comprising the nucleic acid SEQ ID NO. 25283, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 25283 or polypeptide SEQ ID NO. 25284, respectively, or a homolog or a fragment thereof, is increased or generated, or if the activity At5g16650-protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 48 to 142-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a At5g16650-protein, or if the activity of the polypeptide At5g16650, preferably represented by SEQ ID NO. 25284, or a homolog or fragment thereof, or if the activity of a polypeptide encoded by a nucleic acid molecule comprising the nucleic acid SEQ ID NO. 25283, preferably the coding region thereof, or a homolog or fragment thereof, e.g. derived from Arabidopsis thaliana, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 25283 or polypeptide SEQ ID NO. 25284, respectively, or a homolog or a fragment thereof, is increased or generated, or if the activity At5g16650-protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 24 to 86-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a monothiol glutaredoxin, or if the activity of the polypeptide At5g18600, preferably represented by SEQ ID NO. 4905, or a homolog or fragment thereof, or if the activity of a polypeptide encoded by a nucleic acid molecule comprising the nucleic acid SEQ ID NO. 4904, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 4904 or polypeptide SEQ ID NO. 4905, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 31 to 59-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a thioredoxin, or if the activity of the polypeptide At5g39950, preferably represented by SEQ ID NO. 69575, or a homolog or fragment thereof, or if the activity of a polypeptide encoded by a nucleic acid molecule comprising the nucleic acid SEQ ID NO. 69574, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 69574 or polypeptide SEQ ID NO. 69575, respectively, or a homolog or a fragment thereof, is increased or generated, or if the activity thioredoxin is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 53 to 145-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a thioredoxin, or if the activity of the polypeptide At5g39950, preferably represented by SEQ ID NO. 69575, or a homolog or fragment thereof, or if the activity of a polypeptide encoded by a nucleic acid molecule comprising the nucleic acid SEQ ID NO. 69574, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 69574 or polypeptide SEQ ID NO. 69575, respectively, or a homolog or a fragment thereof, is increased or generated, or if the activity thioredoxin is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 23 to 50-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a disease resistance protein, or if the activity of the polypeptide At5g45060, preferably represented by SEQ ID NO. 102224, or a homolog or fragment thereof, or if the activity of a polypeptide encoded by a nucleic acid molecule comprising the nucleic acid SEQ ID NO. 102223, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 102223 or polypeptide SEQ ID NO. 102224, respectively, or a homolog or a fragment thereof, is increased or generated, or if the activity disease resistance protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 24 to 70-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a pyruvate kinase, or if the activity of the polypeptide At5g56350, preferably represented by SEQ ID NO. 102374, or a homolog or fragment thereof, or if the activity of a polypeptide encoded by a nucleic acid molecule comprising the nucleic acid SEQ ID NO. 102373, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 102373 or polypeptide SEQ ID NO. 102374, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line discloses in column 7 the fine chemical isoleucine. For example, an increase of the isoleucine of at least 1 percent, particularly in a range of 35 to 84-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a pyruvate kinase, or if the activity of the polypeptide At5g56350, preferably represented by SEQ ID NO. 102374, or a homolog or fragment thereof, or if the activity of a polypeptide encoded by a nucleic acid molecule comprising the nucleic acid SEQ ID NO. 102373, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 102373 or polypeptide SEQ ID NO. 102374, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 56 to 69-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a RNA-binding protein, or if the activity of the polypeptide At5g60110, preferably represented by SEQ ID NO. 70007, or a homolog or fragment thereof, or if the activity of a polypeptide encoded by a nucleic acid molecule comprising the nucleic acid SEQ ID NO. 70006, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 70006 or polypeptide SEQ ID NO. 70007, respectively, or a homolog or a fragment thereof, is increased or generated, or if the activity

RNA-binding protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 21 to 41-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a MADS-box transcription factor, or if the activity of the polypeptide At5g60440, preferably represented by SEQ ID NO. 102900, or a homolog or fragment thereof, or if the activity of a polypeptide encoded by a nucleic acid molecule comprising the nucleic acid SEQ ID NO. 102899, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 102899 or polypeptide SEQ ID NO. 102900, respectively, or a homolog or a fragment thereof, is increased or generated, or if the activity MADS-box transcription factor is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 33 to 474-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a MADS-box transcription factor, or if the activity of the polypeptide At5g60440, preferably represented by SEQ ID NO. 102900, or a homolog or fragment thereof, or if the activity of a polypeptide encoded by a nucleic acid molecule comprising the nucleic acid SEQ ID NO. 102899, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 102899 or polypeptide SEQ ID NO. 102900, respectively, or a homolog or a fragment thereof, is increased or generated, or if the activity MADS-box transcription factor is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 51 to 643-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a pyruvate kinase, or if the activity of the polypeptide At5g63680, preferably represented by SEQ ID NO. 70039, or a homolog or fragment thereof, or if the activity of a polypeptide encoded by a nucleic acid molecule comprising the nucleic acid SEQ ID NO. 70038, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 70038 or polypeptide SEQ ID NO. 70039, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line discloses in column 7 the fine chemical isoleucine. For example, an increase of the isoleucine of at least 1 percent, particularly in a range of 41 to 182-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a pyruvate kinase, or if the activity of the polypeptide At5g63680, preferably represented by SEQ ID NO. 70039, or a homolog or fragment thereof, or if the activity of a polypeptide encoded by a nucleic acid molecule comprising the nucleic acid SEQ ID NO. 70038, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 70038 or polypeptide SEQ ID NO. 70039, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 55 to 358-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a pyruvate kinase, or if the activity of the polypeptide At5g63680, preferably represented by SEQ ID NO. 70039, or a homolog or fragment thereof, or if the activity of a polypeptide encoded by a nucleic acid molecule comprising the nucleic acid SEQ ID NO. 70038, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 70038 or polypeptide SEQ ID NO. 70039, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line discloses in column 7 the fine chemical valine. For example, an increase of the valine of at least 1-percent, particularly in a range of 31 to 254-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a zinc finger protein, or if the activity of the polypeptide At5g64920, preferably represented by SEQ ID NO. 5494, or a homolog or fragment thereof, or if the activity of a polypeptide encoded by a nucleic acid molecule comprising the nucleic acid SEQ ID NO. 5493, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 5493 or polypeptide SEQ ID NO. 5494, respectively, or a homolog or a fragment thereof, is increased or generated, or if the activity zinc finger protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 35 to 83-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a malic enzyme, or if the activity of the polypeptide AvinDRAFT_{—}1495, preferably represented by SEQ ID NO. 5558, or a homolog or fragment thereof, or if the activity of a polypeptide encoded by a nucleic acid molecule comprising the nucleic acid SEQ ID NO. 5557, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 5557 or polypeptide SEQ ID NO. 5558, respectively, or a homolog or a fragment thereof, is increased or generated, or if the activity malic enzyme is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 85 to 297-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a malic enzyme, or if the activity of the polypeptide AvinDRAFT_{—}1495, preferably represented by SEQ ID NO. 5558, or a homolog or fragment thereof, or if the activity of a polypeptide encoded by a nucleic acid molecule comprising the nucleic acid SEQ ID NO. 5557, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 5557 or polypeptide SEQ ID NO. 5558, respectively, or a homolog or a fragment thereof, is increased or generated, or if the activity malic enzyme is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 98 to 461-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a malic enzyme, or if the activity of the polypeptide AvinDRAFT_{—}1495, preferably represented by SEQ ID NO. 5558, or a homolog or fragment thereof, or if the activity of a polypeptide encoded by a nucleic acid molecule comprising the nucleic acid SEQ ID NO. 5557, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 5557 or polypeptide SEQ ID NO. 5558, respectively, or a homolog or a fragment thereof, is increased or generated, or if the activity malic enzyme is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 50 to 122-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a pyruvate kinase, or if the activity of the polypeptide AvinDRAFT_{—}1806, preferably represented by SEQ ID NO. 26435, or a homolog or fragment thereof, or if the activity of a polypeptide encoded by a nucleic acid molecule comprising the nucleic acid SEQ ID NO. 26434, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 26434 or polypeptide SEQ ID NO. 26435, respectively, or a homolog or a fragment thereof, is increased or generated, or if the activity pyruvate kinase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 21 to 47-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a beta-hydroxylase, or if the activity of the polypeptide AvinDRAFT_{—}2091, preferably represented by SEQ ID NO. 6041, or a homolog or fragment thereof, or if the activity of a polypeptide encoded by a nucleic acid molecule comprising the nucleic acid SEQ ID NO. 6040, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 6040 or polypeptide SEQ ID NO. 6041, respectively, or a homolog or a fragment thereof, is increased or generated, or if the activity beta-hydroxylase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 32 to 131-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a elongation factor Tu, or if the activity of the polypeptide AvinDRAFT_{—}2344, preferably represented by SEQ ID NO. 27022, or a homolog or fragment thereof, or if the activity of a polypeptide encoded by a nucleic acid molecule comprising the nucleic acid SEQ ID NO. 27021, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 27021 or polypeptide SEQ ID NO. 27022, respectively, or a homolog or a fragment thereof, is increased or generated, or if the activity elongation factor Tu is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 43 to 379-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a elongation factor Tu, or if the activity of the polypeptide AvinDRAFT_{—}2344, preferably represented by SEQ ID NO. 27022, or a homolog or fragment thereof, or if the activity of a polypeptide encoded by a nucleic acid molecule comprising the nucleic acid SEQ ID NO. 27021, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 27021 or polypeptide SEQ ID NO. 27022, respectively, or a homolog or a fragment thereof, is increased or generated, or if the activity elongation factor Tu is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 114 to 353-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a elongation factor Tu, or if the activity of the polypeptide AvinDRAFT_{—}2344, preferably represented by SEQ ID NO. 27022, or a homolog or fragment thereof, or if the activity of a polypeptide encoded by a nucleic acid molecule comprising the nucleic acid SEQ ID NO. 27021, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 27021 or polypeptide SEQ ID NO. 27022, respectively, or a homolog or a fragment thereof, is increased or generated, or if the activity elongation factor Tu is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 47 to 203-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a 50S ribosomal protein L14, or if the activity of the polypeptide AvinDRAFT_{—}2369, preferably represented by SEQ ID NO. 71764, or a homolog or fragment thereof, or if the activity of a polypeptide encoded by a nucleic acid molecule comprising the nucleic acid SEQ ID NO. 71763, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 71763 or polypeptide SEQ ID NO. 71764, respectively, or a homolog or a fragment thereof, is increased or generated, or if the activity 50S ribosomal protein L14 is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 22 to 62-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a ABC transporter permease protein, or if the activity of the polypeptide AvinDRAFT_{—}2521, preferably represented by SEQ ID NO. 27883, or a homolog or fragment thereof, or if the activity of a polypeptide encoded by a nucleic acid molecule comprising the nucleic acid SEQ ID NO. 27882, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 27882 or polypeptide SEQ ID NO. 27883, respectively, or a homolog or a fragment thereof, is increased or generated, or if the activity ABC transporter permease protein is increased or generated in a non-human organism, like a microorganism or a plant cell, plant or part thereof, especially with non-targeted localization, whereby the respective line discloses in column 7 the fine chemical valine.

For example, an increase of the valine of at least 1 percent, particularly in a range of 31 to 94-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a enoyl-CoA hydratase, or if the activity of the polypeptide AvinDRAFT_{—}3159, preferably represented by SEQ ID NO. 28739, or a homolog or fragment thereof, or if the activity of a polypeptide encoded by a nucleic acid molecule comprising the nucleic acid SEQ ID NO. 28738, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 28738 or polypeptide SEQ ID NO. 28739, respectively, or a homolog or a fragment thereof, is increased or generated, or if the activity enoyl-CoA hydratase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 114 to 177-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a enoyl-CoA hydratase, or if the activity of the polypeptide AvinDRAFT_{—}3159, preferably represented by SEQ ID NO. 28739, or a homolog or fragment thereof, or if the activity of a polypeptide encoded by a nucleic acid molecule comprising the nucleic acid SEQ ID NO. 28738, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 28738 or polypeptide SEQ ID NO. 28739, respectively, or a homolog or a fragment thereof, is increased or generated, or if the activity enoyl-CoA hydratase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 134 to 194-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a enoyl-CoA hydratase, or if the activity of the polypeptide AvinDRAFT_{—}3159, preferably represented by SEQ ID NO. 28739, or a homolog or fragment thereof, or if the activity of a polypeptide encoded by a nucleic acid molecule comprising the nucleic acid SEQ ID NO. 28738, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 28738 or polypeptide SEQ ID NO. 28739, respectively, or a homolog or a fragment thereof, is increased or generated, or if the activity enoyl-CoA hydratase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 46 to 98-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a transcriptional regulator, or if the activity of the polypeptide AvinDRAFT_{—}3209, preferably represented by SEQ ID NO. 29287, or a homolog or fragment thereof, or if the activity of a polypeptide encoded by a nucleic acid molecule comprising the nucleic acid SEQ ID NO. 29286, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 29286 or polypeptide SEQ ID NO. 29287, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 41 to 207-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a transcriptional regulator, or if the activity of the polypeptide AvinDRAFT_{—}3209, preferably represented by SEQ ID NO. 29287, or a homolog or fragment thereof, or if the activity of a polypeptide encoded by a nucleic acid molecule comprising the nucleic acid SEQ ID NO. 29286, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 29286 or polypeptide SEQ ID NO. 29287, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 75 to 190-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a transcriptional regulator, or if the activity of the polypeptide AvinDRAFT_{—}3209, preferably represented by SEQ ID NO. 29287, or a homolog or fragment thereof, or if the activity of a polypeptide encoded by a nucleic acid molecule comprising the nucleic acid SEQ ID NO. 29286, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 29286 or polypeptide SEQ ID NO. 29287, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 37 to 99-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a acyltransferase, or if the activity of the polypeptide AvinDRAFT_{—}3250, preferably represented by SEQ ID NO. 29398, or a homolog or fragment thereof, or if the activity of a polypeptide encoded by a nucleic acid molecule comprising the nucleic acid SEQ ID NO. 29397, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 29397 or polypeptide SEQ ID NO. 29398, respectively, or a homolog or a fragment thereof, is increased or generated, or if the activity acyltransferase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 120 to 251-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a acyltransferase, or if the activity of the polypeptide AvinDRAFT_{—}3250, preferably represented by SEQ ID NO. 29398, or a homolog or fragment thereof, or if the activity of a polypeptide encoded by a nucleic acid molecule comprising the nucleic acid SEQ ID NO. 29397, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 29397 or polypeptide SEQ ID NO. 29398, respectively, or a homolog or a fragment thereof, is increased or generated, or if the activity acyltransferase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 85 to 298-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a acyltransferase, or if the activity of the polypeptide AvinDRAFT_{—}3250, preferably represented by SEQ ID NO. 29398, or a homolog or fragment thereof, or if the activity of a polypeptide encoded by a nucleic acid molecule comprising the nucleic acid SEQ ID NO. 29397, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 29397 or polypeptide SEQ ID NO. 29398, respectively, or a homolog or a fragment thereof, is increased or generated, or if the activity acyltransferase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 25 to 101-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a oxidoreductase, or if the activity of the polypeptide AvinDRAFT_{—}3253, preferably represented by SEQ ID NO. 29501, or a homolog or fragment thereof, or if the activity of a polypeptide encoded by a nucleic acid molecule comprising the nucleic acid SEQ ID NO. 29500, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 29500 or polypeptide SEQ ID NO. 29501, respectively, or a homolog or a fragment thereof, is increased or generated, or if the activity oxidoreductase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 35 to 67-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a oxidoreductase, or if the activity of the polypeptide AvinDRAFT_{—}3253, preferably represented by SEQ ID NO. 29501, or a homolog or fragment thereof, or if the activity of a polypeptide encoded by a nucleic acid molecule comprising the nucleic acid SEQ ID NO. 29500, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 29500 or polypeptide SEQ ID NO. 29501, respectively, or a homolog or a fragment thereof, is increased or generated, or if the activity oxidoreductase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 51 to 66-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a oxidoreductase, or if the activity of the polypeptide AvinDRAFT_{—}3253, preferably represented by SEQ ID NO. 29501, or a homolog or fragment thereof, or if the activity of a polypeptide encoded by a nucleic acid molecule comprising the nucleic acid SEQ ID NO. 29500, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 29500 or polypeptide SEQ ID NO. 29501, respectively, or a homolog or a fragment thereof, is increased or generated, or if the activity oxidoreductase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 20 to 52-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a ABC transporter component, or if the activity of the polypeptide AVINDRAFT_{—}4128, preferably represented by SEQ ID NO. 72521, or a homolog or fragment thereof, or if the activity of a polypeptide encoded by a nucleic acid molecule comprising the nucleic acid SEQ ID NO. 72520, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 72520 or polypeptide SEQ ID NO. 72521, respectively, or a homolog or a fragment thereof, is increased or generated, or if the activity ABC transporter component is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 22 to 64-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a glucose-1-phosphate cytidylyltransferase, or if the activity of the polypeptide AvinDRAFT_{—}4384, preferably represented by SEQ ID NO. 31718, or a homolog or fragment thereof, or if the activity of a polypeptide encoded by a nucleic acid molecule comprising the nucleic acid SEQ ID NO. 31717, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 31717 or polypeptide SEQ ID NO. 31718, respectively, or a homolog or a fragment thereof, is increased or generated, or if the activity glucose-1-phosphate cytidylyltransferase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 24 to 32-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a aminotransferase, or if the activity of the polypeptide AvinDRAFT_{—}4562, preferably represented by SEQ ID NO. 31927, or a homolog or fragment thereof, or if the activity of a polypeptide encoded by a nucleic acid molecule comprising the nucleic acid SEQ ID NO. 31926, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 31926 or polypeptide SEQ ID NO. 31927, respectively, or a homolog or a fragment thereof, is increased or generated, or if the activity aminotransferase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 59 to 77-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a aminotransferase, or if the activity of the polypeptide AvinDRAFT_{—}4562, preferably represented by SEQ ID NO. 31927, or a homolog or fragment thereof, or if the activity of a polypeptide encoded by a nucleic acid molecule comprising the nucleic acid SEQ ID NO. 31926, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 31926 or polypeptide SEQ ID NO. 31927, respectively, or a homolog or a fragment thereof, is increased or generated, or if the activity aminotransferase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 26 to 56-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a NADH-quinone oxidoreductase subunit, or if the activity of the polypeptide AvinDRAFT_{—}4606, preferably represented by SEQ ID NO. 73720, or a homolog or fragment thereof, or if the activity of a polypeptide encoded by a nucleic acid molecule comprising the nucleic acid SEQ ID NO. 73719, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 73719 or polypeptide SEQ ID NO. 73720, respectively, or a homolog or a fragment thereof, is increased or generated, or if the activity NADH-quinone oxidoreductase subunit is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 40 to 127-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a NADH-quinone oxidoreductase subunit, or if the activity of the polypeptide AvinDRAFT_{—}4606, preferably represented by SEQ ID NO. 73720, or a homolog or fragment thereof, or if the activity of a polypeptide encoded by a nucleic acid molecule comprising the nucleic acid SEQ ID NO. 73719, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 73719 or polypeptide SEQ ID NO. 73720, respectively, or a homolog or a fragment thereof, is increased or generated, or if the activity NADH-quinone oxidoreductase subunit is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 58 to 85-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a glutamate-ammonia-ligase, or if the activity of the polypeptide AvinDRAFT_{—}4836, preferably represented by SEQ ID NO. 74164, or a homolog or fragment thereof, or if the activity of a polypeptide encoded by a nucleic acid molecule comprising the nucleic acid SEQ ID NO. 74163, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 74163 or polypeptide SEQ ID NO. 74164, respectively, or a homolog or a fragment thereof, is increased or generated, or if the activity glutamate-ammonia-ligase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 64 to 213-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a glutamate-ammonialigase, or if the activity of the polypeptide AvinDRAFT_{—}4836, preferably represented by SEQ ID NO. 74164, or a homolog or fragment thereof, or if the activity of a polypeptide encoded by a nucleic acid molecule comprising the nucleic acid SEQ ID NO. 74163, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 74163 or polypeptide SEQ ID NO. 74164, respectively, or a homolog or a fragment thereof, is increased or generated, or if the activity glutamate-ammonia-ligase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 68 to 176-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a glutamate-ammonia-ligase, or if the activity of the polypeptide AvinDRAFT_{—}4836, preferably represented by SEQ ID NO. 74164, or a homolog or fragment thereof, or if the activity of a polypeptide encoded by a nucleic acid molecule comprising the nucleic acid SEQ ID NO. 74163, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 74163 or polypeptide SEQ ID NO. 74164, respectively, or a homolog or a fragment thereof, is increased or generated, or if the activity glutamate-ammonia-ligase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 24 to 135-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a isopropylmalate isomerase large subunit, or if the activity of the polypeptide AvinDRAFT_{—}4847, preferably represented by SEQ ID NO. 102942, or a homolog or fragment thereof, or if the activity of a polypeptide encoded by a nucleic acid molecule comprising the nucleic acid SEQ ID NO. 102941, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 102941 or polypeptide SEQ ID NO. 102942, respectively, or a homolog or a fragment thereof, is increased or generated, or if the activity isopropylmalate isomerase large subunit is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 26 to 31-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a hydrolase, or if the activity of the polypeptide AvinDRAFT_{—}5103, preferably represented by SEQ ID NO. 6511, or a homolog or fragment thereof, or if the activity of a polypeptide encoded by a nucleic acid molecule comprising the nucleic acid SEQ ID NO. 6510, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 6510 or polypeptide SEQ ID NO. 6511, respectively, or a homolog or a fragment thereof, is increased or generated, or if the activity hydrolase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 41 to 2397-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a hydrolase, or if the activity of the polypeptide AvinDRAFT_{—}5103, preferably represented by SEQ ID NO. 6511, or a homolog or fragment thereof, or if the activity of a polypeptide encoded by a nucleic acid molecule comprising the nucleic acid SEQ ID NO. 6510, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 6510 or polypeptide SEQ ID NO. 6511, respectively, or a homolog or a fragment thereof, is increased or generated, or if the activity hydrolase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 51 to 2753-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a hydrolase, or if the activity of the polypeptide AvinDRAFT_{—}5103, preferably represented by SEQ ID NO. 6511, or a homolog or fragment thereof, or if the activity of a polypeptide encoded by a nucleic acid molecule comprising the nucleic acid SEQ ID NO. 6510, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 6510 or polypeptide SEQ ID NO. 6511, respectively, or a homolog or a fragment thereof, is increased or generated, or if the activity hydrolase is increased or generated in a non-human organism, like a microorganism or a plant cell, plant or part thereof, especially with non-targeted localization, whereby the respective line discloses in column 7 the fine chemical valine. For example, an increase of the valine of at least 1-percent, particularly in a range of 24 to 951-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a fumarylacetoacetate hydrolase, or if the activity of the polypeptide AvinDRAFT_{—}5292, preferably represented by SEQ ID NO. 32309, or a homolog or fragment thereof, or if the activity of a polypeptide encoded by a nucleic acid molecule comprising the nucleic acid SEQ ID NO. 32308, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 32308 or polypeptide SEQ ID NO. 32309, respectively, or a homolog or a fragment thereof, is increased or generated, or if the activity fumarylacetoacetate hydrolase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 335 to 446-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a fumarylacetoacetate hydrolase, or if the activity of the polypeptide AvinDRAFT_{—}5292, preferably represented by SEQ ID NO. 32309, or a homolog or fragment thereof, or if the activity of a polypeptide encoded by a nucleic acid molecule comprising the nucleic acid SEQ ID NO. 32308, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 32308 or polypeptide SEQ ID NO. 32309, respectively, or a homolog or a fragment thereof, is increased or generated, or if the activity fumarylacetoacetate hydrolase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 469 to 550-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a fumarylacetoacetate hydrolase, or if the activity of the polypeptide AvinDRAFT_{—}5292, preferably represented by SEQ ID NO. 32309, or a homolog or fragment thereof, or if the activity of a polypeptide encoded by a nucleic acid molecule comprising the nucleic acid SEQ ID NO. 32308, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 32308 or polypeptide SEQ ID NO. 32309, respectively, or a homolog or a fragment thereof, is increased or generated, or if the activity fumarylacetoacetate hydrolase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 132 to 187 -percent is conferred as compared to a corresponding non-transformed wild type non-human organism

Accordingly, in one embodiment, an increase of leucine in a non-human organism, as compared to a corresponding non-transformed wild type non-human organism, is conferred in the process of the invention, if the activity of a polypeptide showing the activity of a peptidase, or if the activity of the polypeptide AvinDRAFT_{—}5579, preferably represented by SEQ ID NO. 74663, or a homolog or fragment thereof, or if the activity of a polypeptide encoded by a nucleic acid molecule comprising the nucleic acid SEQ ID NO. 74662, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 74662 or polypeptide SEQ ID NO. 74663, respectively, or a homolog or a fragment thereof, is increased or generated, or if the activity peptidase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 57 to 94-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a chaperone protein CIpB, or if the activity of the polypeptide AvinDRAFT_{—}6093, preferably represented by SEQ ID NO. 33597, or a homolog or fragment thereof, or if the activity of a polypeptide encoded by a nucleic acid molecule comprising the nucleic acid SEQ ID NO. 33596, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 33596 or polypeptide SEQ ID NO. 33597, respectively, or a homolog or a fragment thereof, is increased or generated, or if the activity chaperone protein CIpB is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 44 to 134-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a purine nucleoside phosphorylase, or if the activity of the polypeptide AvinDRAFT_{—}6700, preferably represented by SEQ ID NO. 34045, or a homolog or fragment thereof, or if the activity of a polypeptide encoded by a nucleic acid molecule comprising the nucleic acid SEQ ID NO. 34044, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 34044 or polypeptide SEQ ID NO. 34045, respectively, or a homolog or a fragment thereof, is increased or generated, or if the activity purine nucleoside phosphorylase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 32 to 34-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a purine nucleoside phosphorylase, or if the activity of the polypeptide AvinDRAFT_{—}6700, preferably represented by SEQ ID NO. 34045, or a homolog or fragment thereof, or if the activity of a polypeptide encoded by a nucleic acid molecule comprising the nucleic acid SEQ ID NO. 34044, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 34044 or polypeptide SEQ ID NO. 34045, respectively, or a homolog or a fragment thereof, is increased or generated, or if the activity purine nucleoside phosphorylase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 31 to 53-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a purine nucleoside phosphorylase, or if the activity of the polypeptide AvinDRAFT_{—}6700, preferably represented by SEQ ID NO. 34045, or a homolog or fragment thereof, or if the activity of a polypeptide encoded by a nucleic acid molecule comprising the nucleic acid SEQ ID NO. 34044, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 34044 or polypeptide SEQ ID NO. 34045, respectively, or a homolog or a fragment thereof, is increased or generated, or if the activity purine nucleoside phosphorylase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 22 to 29-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a pyruvate kinase, or if the activity of the polypeptide AY623894, preferably represented by SEQ ID NO. 103434, or a homolog or fragment thereof, or if the activity of a polypeptide encoded by a nucleic acid molecule comprising the nucleic acid SEQ ID NO. 103433, preferably the coding region thereof, or a homolog or fragment thereof, e.g. derived from Zea mays, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 103433 or polypeptide SEQ ID NO. 103434, respectively, or a homolog or a fragment thereof, is increased or generated, or if the activity pyruvate kinase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 49 to 103-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a pyruvate kinase, or if the activity of the polypeptide AY623894, preferably represented by SEQ ID NO. 103434, or a homolog or fragment thereof, or if the activity of a polypeptide encoded by a nucleic acid molecule comprising the nucleic acid SEQ ID NO. 103433, preferably the coding region thereof, or a homolog or fragment thereof, e.g. derived from Zea mays, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 103433 or polypeptide SEQ ID NO. 103434, respectively, or a homolog or a fragment thereof, is increased or generated, or if the activity pyruvate kinase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 30 to 46-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a threonine synthase, or if the activity of the polypeptide B0004, preferably represented by SEQ ID NO. 34890, or a homolog or fragment thereof, or if the activity of a polypeptide encoded by a nucleic acid molecule comprising the nucleic acid SEQ ID NO. 34889, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 34889 or polypeptide SEQ ID NO. 34890, respectively, or a homolog or a fragment thereof, is increased or generated, or if the activity threonine synthase is increased or generated in a non-human organism, like a microorganism or a plant cell, plant or part thereof, especially with plastidic localization, whereby the respective line discloses in column 7 the fine chemical isoleucine. For example, an increase of the isoleucine of at least 1 percent, particularly in a range of 40 to 468-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a threonine synthase, or if the activity of the polypeptide B0004, preferably represented by SEQ ID NO. 34890, or a homolog or fragment thereof, or if the activity of a polypeptide encoded by a nucleic acid molecule comprising the nucleic acid SEQ ID NO. 34889, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 34889 or polypeptide SEQ ID NO. 34890, respectively, or a homolog or a fragment thereof, is increased or generated, or if the activity threonine synthase is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 96 to 297-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a threonine synthase, or if the activity of the polypeptide B0004, preferably represented by SEQ ID NO. 34890, or a homolog or fragment thereof, or if the activity of a polypeptide encoded by a nucleic acid molecule comprising the nucleic acid SEQ ID NO. 34889, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 34889 or polypeptide SEQ ID NO. 34890, respectively, or a homolog or a fragment thereof, is increased or generated, or if the activity threonine synthase is increased or generated in a non-human organism, like a microorganism or a plant cell, plant or part thereof, especially with plastidic localization, whereby the respective line discloses in column 7 the fine chemical valine. For example, an increase of the valine of at least 1 percent, particularly in a range of 33 to 220-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a b0050-protein, or if the activity of the polypeptide B0050, preferably represented by SEQ ID NO. 103959, or a homolog or fragment thereof, or if the activity of a polypeptide encoded by a nucleic acid molecule comprising the nucleic acid SEQ ID NO. 103958, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 103958 or polypeptide SEQ ID NO. 103959, respectively, or a homolog or a fragment thereof, is increased or generated, or if the activity b0050-protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 24 to 122-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a acetolactate synthase small subunit, or if the activity of the polypeptide B0078, preferably represented by SEQ ID NO. 6819, or a homolog or fragment thereof, or if the activity of a polypeptide encoded by a nucleic acid molecule comprising the nucleic acid SEQ ID NO. 6818, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 6818 or polypeptide SEQ ID NO. 6819, respectively, or a homolog or a fragment thereof, is increased or generated, or if the activity acetolactate synthase small subunit is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 44 to 73-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a beta-galactosidase, or if the activity of the polypeptide B0344, preferably represented by SEQ ID NO. 35734, or a homolog or fragment thereof, or if the activity of a polypeptide encoded by a nucleic acid molecule comprising the nucleic acid SEQ ID NO. 35733, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 35733 or polypeptide SEQ ID NO. 35734, respectively, or a homolog or a fragment thereof, is increased or generated, or if the activity beta-galactosidase is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 224 to 288-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a 2,3-dihydroxyphenylpropionate 1,2-dioxygenase, or if the activity of the polypeptide B0348, preferably represented by SEQ ID NO. 7270, or a homolog or fragment thereof, or if the activity of a polypeptide encoded by a nucleic acid molecule comprising the nucleic acid SEQ ID NO. 7269, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 7269 or polypeptide SEQ ID NO. 7270, respectively, or a homolog or a fragment thereof, is increased or generated, or if the activity 2,3-dihydroxyphenylpropionate 1,2-dioxygenase is increased or generated in a non-human organism, like a microorganism or a plant cell, plant or part thereof, especially with plastidic localization, whereby the respective line discloses in column 7 the fine chemical isoleucine. For example, an increase of the isoleucine of at least 1 percent, particularly in a range of 64 to 316-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a 2,3-dihydroxyphenylpropionate 1,2-dioxygenase, or if the activity of the polypeptide B0348, preferably represented by SEQ ID NO. 7270, or a homolog or fragment thereof, or if the activity of a polypeptide encoded by a nucleic acid molecule comprising the nucleic acid SEQ ID NO. 7269, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 7269 or polypeptide SEQ ID NO. 7270, respectively, or a homolog or a fragment thereof, is increased or generated, or if the activity 2,3-dihydroxyphenylpropionate 1,2-dioxygenase is increased or generated in a non-human organism, like a microorganism or a plant cell, plant or part thereof, especially with plastidic localization, whereby the respective line discloses in column 7 the fine chemical valine. For example, an increase of the valine of at least 1 percent, particularly in a range of 35 to 236-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a ATP-binding component of a transport system, or if the activity of the polypeptide B0449, preferably represented by SEQ ID NO. 7334, or a homolog or fragment thereof, or if the activity of a polypeptide encoded by a nucleic acid molecule comprising the nucleic acid SEQ ID NO. 7333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 7333 or polypeptide SEQ ID NO. 7334, respectively, or a homolog or a fragment thereof, is increased or generated, or if the activity ATP-binding component of a transport system is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 75 to 2019-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a ATP-binding component of a transport system, or if the activity of the polypeptide B0449, preferably represented by SEQ ID NO. 7334, or a homolog or fragment thereof, or if the activity of a polypeptide encoded by a nucleic acid molecule comprising the nucleic acid SEQ ID NO. 7333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 7333 or polypeptide SEQ ID NO. 7334, respectively, or a homolog or a fragment thereof, is increased or generated, or if the activity ATP-binding component of a transport system is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 82 to 2204-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a ATP-binding component of a transport system, or if the activity of the polypeptide B0449, preferably represented by SEQ ID NO. 7334, or a homolog or fragment thereof, or if the activity of a polypeptide encoded by a nucleic acid molecule comprising the nucleic acid SEQ ID NO. 7333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 7333 or polypeptide SEQ ID NO. 7334, respectively, or a homolog or a fragment thereof, is increased or generated, or if the activity ATP-binding component of a transport system is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 21 to 774-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a membrane transport protein, or if the activity of the polypeptide B0486, preferably represented by SEQ ID NO. 7687, or a homolog or fragment thereof, or if the activity of a polypeptide encoded by a nucleic acid molecule comprising the nucleic acid SEQ ID NO. 7686, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 7686 or polypeptide SEQ ID NO. 7687, respectively, or a homolog or a fragment thereof, is increased or generated, or if the activity membrane transport protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 68 to 394-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a isochorismate synthase, or if the activity of the polypeptide B0593, preferably represented by SEQ ID NO. 35968, or a homolog or fragment thereof, or if the activity of a polypeptide encoded by a nucleic acid molecule comprising the nucleic acid SEQ ID NO. 35967, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 35967 or polypeptide SEQ ID NO. 35968, respectively, or a homolog or a fragment thereof, is increased or generated, or if the activity isochorismate synthase is increased or generated in a non-human organism, like a microorganism or a plant cell, plant or part thereof, especially with plastidic localization, whereby the respective line discloses in column 7 the fine chemical isoleucine. For example, an increase of the isoleucine of at least 1 percent, particularly in a range of 77 to 474-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a isochorismate synthase, or if the activity of the polypeptide B0593, preferably represented by SEQ ID NO. 35968, or a homolog or fragment thereof, or if the activity of a polypeptide encoded by a nucleic acid molecule comprising the nucleic acid SEQ ID NO. 35967, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 35967 or polypeptide SEQ ID NO. 35968, respectively, or a homolog or a fragment thereof, is increased or generated, or if the activity isochorismate synthase is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 74 to 478-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a isochorismate synthase, or if the activity of the polypeptide B0593, preferably represented by SEQ ID NO. 35968, or a homolog or fragment thereof, or if the activity of a polypeptide encoded by a nucleic acid molecule comprising the nucleic acid SEQ ID NO. 35967, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 35967 or polypeptide SEQ ID NO. 35968, respectively, or a homolog or a fragment thereof, is increased or generated, or if the activity isochorismate synthase is increased or generated in a non-human organism, like a microorganism or a plant cell, plant or part thereof, especially with plastidic localization, whereby the respective line discloses in column 7 the fine chemical valine. For example, an increase of the valine of at least 1 percent, particularly in a range of 62 to 214-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a zinc transporter, or if the activity of the polypeptide B0752, preferably represented by SEQ ID NO. 36115, or a homolog or fragment thereof, or if the activity of a polypeptide encoded by a nucleic acid molecule comprising the nucleic acid SEQ ID NO. 36114, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 36114 or polypeptide SEQ ID NO. 36115, respectively, or a homolog or a fragment thereof, is increased or generated, or if the activity zinc transporter is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 36 to 58-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a zinc transporter, or if the activity of the polypeptide B0752, preferably represented by SEQ ID NO. 36115, or a homolog or fragment thereof, or if the activity of a polypeptide encoded by a nucleic acid molecule comprising the nucleic acid SEQ ID NO. 36114, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 36114 or polypeptide SEQ ID NO. 36115, respectively, or a homolog or a fragment thereof, is increased or generated, or if the activity zinc transporter is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 54 to 81-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a major facilitator superfamily transporter protein, or if the activity of the polypeptide B0898, preferably represented by SEQ ID NO. 7918, or a homolog or fragment thereof, or if the activity of a polypeptide encoded by a nucleic acid molecule comprising the nucleic acid SEQ ID NO. 7917, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 7917 or polypeptide SEQ ID NO. 7918, respectively, or a homolog or a fragment thereof, is increased or generated, or if the activity major facilitator superfamily transporter protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 40 to 6183-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a major facilitator superfamily transporter protein, or if the activity of the polypeptide B0898, preferably represented by SEQ ID NO. 7918, or a homolog or fragment thereof, or if the activity of a polypeptide encoded by a nucleic acid molecule comprising the nucleic acid SEQ ID NO. 7917, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 7917 or polypeptide SEQ ID NO. 7918, respectively, or a homolog or a fragment thereof, is increased or generated, or if the activity major facilitator superfamily transporter protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 59 to 5609-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a major facilitator superfamily transporter protein, or if the activity of the polypeptide B0898, preferably represented by SEQ ID NO. 7918, or a homolog or fragment thereof, or if the activity of a polypeptide encoded by a nucleic acid molecule comprising the nucleic acid SEQ ID NO. 7917, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 7917 or polypeptide SEQ ID NO. 7918, respectively, or a homolog or a fragment thereof, is increased or generated, or if the activity major facilitator superfamily transporter protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 23 to 2254-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a DNA helicase IV, or if the activity of the polypeptide B0962, preferably represented by SEQ ID NO. 36624, or a homolog or fragment thereof, or if the activity of a polypeptide encoded by a nucleic acid molecule comprising the nucleic acid SEQ ID NO. 36623, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 36623 or polypeptide SEQ ID NO. 36624, respectively, or a homolog or a fragment thereof, is increased or generated, or if the activity DNA helicase IV is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 52 to 274-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a methylglyoxal synthase, or if the activity of the polypeptide B0963, preferably represented by SEQ ID NO. 36671, or a homolog or fragment thereof, or if the activity of a polypeptide encoded by a nucleic acid molecule comprising the nucleic acid SEQ ID NO. 36670, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 36670 or polypeptide SEQ ID NO. 36671, respectively, or a homolog or a fragment thereof, is increased or generated, or if the activity methylglyoxal synthase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 34 to 127-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a phosphoanhydride phosphorylase, or if the activity of the polypeptide B0980, preferably represented by SEQ ID NO. 36810, or a homolog or fragment thereof, or if the activity of a polypeptide encoded by a nucleic acid molecule comprising the nucleic acid SEQ ID NO. 36809, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 36809 or polypeptide SEQ ID NO. 36810, respectively, or a homolog or a fragment thereof, is increased or generated, or if the activity phosphoanhydride phosphorylase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 33 to 99-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a b1003-protein, or if the activity of the polypeptide B1003, preferably represented by SEQ ID NO. 7942, or a homolog or fragment thereof, or if the activity of a polypeptide encoded by a nucleic acid molecule comprising the nucleic acid SEQ ID NO. 7941, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 7941 or polypeptide SEQ ID NO. 7942, respectively, or a homolog or a fragment thereof, is increased or generated, or if the activity b1003-protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 185 to 1202-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a b1003-protein, or if the activity of the polypeptide B1003, preferably represented by SEQ ID NO. 7942, or a homolog or fragment thereof, or if the activity of a polypeptide encoded by a nucleic acid molecule comprising the nucleic acid SEQ ID NO. 7941, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 7941 or polypeptide SEQ ID NO. 7942, respectively, or a homolog or a fragment thereof, is increased or generated, or if the activity b1003-protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 183 to 1058-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a b1003-protein, or if the activity of the polypeptide B1003, preferably represented by SEQ ID NO. 7942, or a homolog or fragment thereof, or if the activity of a polypeptide encoded by a nucleic acid molecule comprising the nucleic acid SEQ ID NO. 7941, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 7941 or polypeptide SEQ ID NO. 7942, respectively, or a homolog or a fragment thereof, is increased or generated, or if the activity b1003-protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 71 to 549-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a b1163-protein, or if the activity of the polypeptide B1163, preferably represented by SEQ ID NO. 37395, or a homolog or fragment thereof, or if the activity of a polypeptide encoded by a nucleic acid molecule comprising the nucleic acid SEQ ID NO. 37394, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 37394 or polypeptide SEQ ID NO. 37395, respectively, or a homolog or a fragment thereof, is increased or generated, or if the activity b1163-protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 33 to 107-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a b1163-protein, or if the activity of the polypeptide B1163, preferably represented by SEQ ID NO. 37395, or a homolog or fragment thereof, or if the activity of a polypeptide encoded by a nucleic acid molecule comprising the nucleic acid SEQ ID NO. 37394, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 37394 or polypeptide SEQ ID NO. 37395, respectively, or a homolog or a fragment thereof, is increased or generated, or if the activity b1163-protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 53 to 134-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a b1163-protein, or if the activity of the polypeptide B1163, preferably represented by SEQ ID NO. 37395, or a homolog or fragment thereof, or if the activity of a polypeptide encoded by a nucleic acid molecule comprising the nucleic acid SEQ ID NO. 37394, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 37394 or polypeptide SEQ ID NO. 37395, respectively, or a homolog or a fragment thereof, is increased or generated, or if the activity b1163-protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 27 to 47-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a DNA polymerase, or if the activity of the polypeptide B1184, preferably represented by SEQ ID NO. 104135, or a homolog or fragment thereof, or if the activity of a polypeptide encoded by a nucleic acid molecule comprising the nucleic acid SEQ ID NO. 104134, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 104134 or polypeptide SEQ ID NO. 104135, respectively, or a homolog or a fragment thereof, is increased or generated, or if the activity DNA polymerase is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 31 to 169-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a DNA polymerase, or if the activity of the polypeptide B1184, preferably represented by SEQ ID NO. 104135, or a homolog or fragment thereof, or if the activity of a polypeptide encoded by a nucleic acid molecule comprising the nucleic acid SEQ ID NO. 104134, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 104134 or polypeptide SEQ ID NO. 104135, respectively, or a homolog or a fragment thereof, is increased or generated, or if the activity DNA polymerase is increased or generated in a non-human organism, like a microorganism or a plant cell, plant or part thereof, especially with plastidic localization, whereby the respective line discloses in column 7 the fine chemical valine. For example, an increase of the valine of at least 1 percent, particularly in a range of 30 to 30-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a membrane protein, or if the activity of the polypeptide B1255, preferably represented by SEQ ID NO. 37484, or a homolog or fragment thereof, or if the activity of a polypeptide encoded by a nucleic acid molecule comprising the nucleic acid SEQ ID NO. 37483, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 37483 or polypeptide SEQ ID NO. 37484, respectively, or a homolog or a fragment thereof, is increased or generated, or if the activity membrane protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 45 to 155-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a membrane protein, or if the activity of the polypeptide B1255, preferably represented by SEQ ID NO. 37484, or a homolog or fragment thereof, or if the activity of a polypeptide encoded by a nucleic acid molecule comprising the nucleic acid SEQ ID NO. 37483, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 37483 or polypeptide SEQ ID NO. 37484, respectively, or a homolog or a fragment thereof, is increased or generated, or if the activity membrane protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 54 to 178-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a membrane protein, or if the activity of the polypeptide B1255, preferably represented by SEQ ID NO. 37484, or a homolog or fragment thereof, or if the activity of a polypeptide encoded by a nucleic acid molecule comprising the nucleic acid SEQ ID NO. 37483, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 37483 or polypeptide SEQ ID NO. 37484, respectively, or a homolog or a fragment thereof, is increased or generated, or if the activity membrane protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 20 to 69-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a anthranilate synthase component II, or if the activity of the polypeptide B1263, preferably represented by SEQ ID NO. 37540, or a homolog or fragment thereof, or if the activity of a polypeptide encoded by a nucleic acid molecule comprising the nucleic acid SEQ ID NO. 37539, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 37539 or polypeptide SEQ ID NO. 37540, respectively, or a homolog or a fragment thereof, is increased or generated, or if the activity anthranilate synthase component II is increased or generated in a non-human organism, like a microorganism or a plant cell, plant or part thereof, especially with plastidic localization, whereby the respective line discloses in column 7 the fine chemical isoleucine. For example, an increase of the isoleucine of at least 1 percent, particularly in a range of 22 to 106-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a anthranilate synthase component II, or if the activity of the polypeptide B1263, preferably represented by SEQ ID NO. 37540, or a homolog or fragment thereof, or if the activity of a polypeptide encoded by a nucleic acid molecule comprising the nucleic acid SEQ ID NO. 37539, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 37539 or polypeptide SEQ ID NO. 37540, respectively, or a homolog or a fragment thereof, is increased or generated, or if the activity anthranilate synthase component II is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 58 to 74-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a anthranilate synthase component II, or if the activity of the polypeptide B1263, preferably represented by SEQ ID NO. 37540, or a homolog or fragment thereof, or if the activity of a polypeptide encoded by a nucleic acid molecule comprising the nucleic acid SEQ ID NO. 37539, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 37539 or polypeptide SEQ ID NO. 37540, respectively, or a homolog or a fragment thereof, is increased or generated, or if the activity anthranilate synthase component II is increased or generated in a non-human organism, like a microorganism or a plant cell, plant or part thereof, especially with plastidic localization, whereby the respective line discloses in column 7 the fine chemical valine. For example, an increase of the valine of at least 1 percent, particularly in a range of 29 to 83-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a glutamine synthetase, or if the activity of the polypeptide B1297, preferably represented by SEQ ID NO. 37659, or a homolog or fragment thereof, or if the activity of a polypeptide encoded by a nucleic acid molecule comprising the nucleic acid SEQ ID NO. 37658, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 37658 or polypeptide SEQ ID NO. 37659, respectively, or a homolog or a fragment thereof, is increased or generated, or if the activity glutamine synthetase is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 195 to 355-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a aldehyde dehydrogenase, or if the activity of the polypeptide B1300, preferably represented by SEQ ID NO. 37808, or a homolog or fragment thereof, or if the activity of a polypeptide encoded by a nucleic acid molecule comprising the nucleic acid SEQ ID NO. 37807, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 37807 or polypeptide SEQ ID NO. 37808, respectively, or a homolog or a fragment thereof, is increased or generated, or if the activity aldehyde dehydrogenase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 20 to 52-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a b1445-protein, or if the activity of the polypeptide B1445, preferably represented by SEQ ID NO. 38290, or a homolog or fragment thereof, or if the activity of a polypeptide encoded by a nucleic acid molecule comprising the nucleic acid SEQ ID NO. 38289, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 38289 or polypeptide SEQ ID NO. 38290, respectively, or a homolog or a fragment thereof, is increased or generated, or if the activity b1445-protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 20 to 49-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a NADP-dependent malic enzyme, or if the activity of the polypeptide B1479, preferably represented by SEQ ID NO. 77775, or a homolog or fragment thereof, or if the activity of a polypeptide encoded by a nucleic acid molecule comprising the nucleic acid SEQ ID NO. 77774, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 77774 or polypeptide SEQ ID NO. 77775, respectively, or a homolog or a fragment thereof, is increased or generated, or if the activity NADP-dependent malic enzyme is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 40 to 188-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a b1522-protein, or if the activity of the polypeptide B1522, preferably represented by SEQ ID NO. 7948, or a homolog or fragment thereof, or if the activity of a polypeptide encoded by a nucleic acid molecule comprising the nucleic acid SEQ ID NO. 7947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 7947 or polypeptide SEQ ID NO. 7948, respectively, or a homolog or a fragment thereof, is increased or generated, or if the activity b1522-protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 41 to 920-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a b1522-protein, or if the activity of the polypeptide B1522, preferably represented by SEQ ID NO. 7948, or a homolog or fragment thereof, or if the activity of a polypeptide encoded by a nucleic acid molecule comprising the nucleic acid SEQ ID NO. 7947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 7947 or polypeptide SEQ ID NO. 7948, respectively, or a homolog or a fragment thereof, is increased or generated, or if the activity b1522-protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 213 to 780-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a b1522-protein, or if the activity of the polypeptide B1522, preferably represented by SEQ ID NO. 7948, or a homolog or fragment thereof, or if the activity of a polypeptide encoded by a nucleic acid molecule comprising the nucleic acid SEQ ID NO. 7947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 7947 or polypeptide SEQ ID NO. 7948, respectively, or a homolog or a fragment thereof, is increased or generated, or if the activity b1522-protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 20 to 336-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a acid shock protein, or if the activity of the polypeptide B1597, preferably represented by SEQ ID NO. 38301, or a homolog or fragment thereof, or if the activity of a polypeptide encoded by a nucleic acid molecule comprising the nucleic acid SEQ ID NO. 38300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 38300 or polypeptide SEQ ID NO. 38301, respectively, or a homolog or a fragment thereof, is increased or generated, or if the activity acid shock protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 69 to 218-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a acid shock protein, or if the activity of the polypeptide B1597, preferably represented by SEQ ID NO. 38301, or a homolog or fragment thereof, or if the activity of a polypeptide encoded by a nucleic acid molecule comprising the nucleic acid SEQ ID NO. 38300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 38300 or polypeptide SEQ ID NO. 38301, respectively, or a homolog or a fragment thereof, is increased or generated, or if the activity acid shock protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 75 to 252-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a acid shock protein, or if the activity of the polypeptide B1597, preferably represented by SEQ ID NO. 38301, or a homolog or fragment thereof, or if the activity of a polypeptide encoded by a nucleic acid molecule comprising the nucleic acid SEQ ID NO. 38300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 38300 or polypeptide SEQ ID NO. 38301, respectively, or a homolog or a fragment thereof, is increased or generated, or if the activity acid shock protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 44 to 105-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a transport protein, or if the activity of the polypeptide B1601, preferably represented by SEQ ID NO. 7993, or a homolog or fragment thereof, or if the activity of a polypeptide encoded by a nucleic acid molecule comprising the nucleic acid SEQ ID NO. 7992, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 7992 or polypeptide SEQ ID NO. 7993, respectively, or a homolog or a fragment thereof, is increased or generated, or if the activity transport protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 21 to 42-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a b1670-protein, or if the activity of the polypeptide B1670, preferably represented by SEQ ID NO. 78754, or a homolog or fragment thereof, or if the activity of a polypeptide encoded by a nucleic acid molecule comprising the nucleic acid SEQ ID NO. 78753, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 78753 or polypeptide SEQ ID NO. 78754, respectively, or a homolog or a fragment thereof, is increased or generated, or if the activity b1670-protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 20 to 80-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a b1672-protein, or if the activity of the polypeptide B1672, preferably represented by SEQ ID NO. 78772, or a homolog or fragment thereof, or if the activity of a polypeptide encoded by a nucleic acid molecule comprising the nucleic acid SEQ ID NO. 78771, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 78771 or polypeptide SEQ ID NO. 78772, respectively, or a homolog or a fragment thereof, is increased or generated, or if the activity b1672-protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 23 to 90-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a pyruvate kinase, or if the activity of the polypeptide B1854, preferably represented by SEQ ID NO. 8364, or a homolog or fragment thereof, or if the activity of a polypeptide encoded by a nucleic acid molecule comprising the nucleic acid SEQ ID NO. 8363, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 8363 or polypeptide SEQ ID NO. 8364, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 58 to 227-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a cytochrome c-type protein, or if the activity of the polypeptide B1873, preferably represented by SEQ ID NO. 78971, or a homolog or fragment thereof, or if the activity of a polypeptide encoded by a nucleic acid molecule comprising the nucleic acid SEQ ID NO. 78970, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 78970 or polypeptide SEQ ID NO. 78971, respectively, or a homolog or a fragment thereof, is increased or generated, or if the activity cytochrome c-type protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 24 to 49-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a trehalosephosphatase, or if the activity of the polypeptide B1897, preferably represented by SEQ ID NO. 79057, or a homolog or fragment thereof, or if the activity of a polypeptide encoded by a nucleic acid molecule comprising the nucleic acid SEQ ID NO. 79056, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 79056 or polypeptide SEQ ID NO. 79057, respectively, or a homolog or a fragment thereof, is increased or generated, or if the activity trehalose-phosphatase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 41 to 333-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a trehalose-phosphatase, or if the activity of the polypeptide B1897, preferably represented by SEQ ID NO. 79057, or a homolog or fragment thereof, or if the activity of a polypeptide encoded by a nucleic acid molecule comprising the nucleic acid SEQ ID NO. 79056, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 79056 or polypeptide SEQ ID NO. 79057, respectively, or a homolog or a fragment thereof, is increased or generated, or if the activity trehalose-phosphatase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 154 to 467-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a trehalose-phosphatase, or if the activity of the polypeptide B1897, preferably represented by SEQ ID NO. 79057, or a homolog or fragment thereof, or if the activity of a polypeptide encoded by a nucleic acid molecule comprising the nucleic acid SEQ ID NO. 79056, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 79056 or polypeptide SEQ ID NO. 79057, respectively, or a homolog or a fragment thereof, is increased or generated, or if the activity trehalose-phosphatase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 38 to 159-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a O-antigen chain length determinant, or if the activity of the polypeptide B2027, preferably represented by SEQ ID NO. 79218, or a homolog or fragment thereof, or if the activity of a polypeptide encoded by a nucleic acid molecule comprising the nucleic acid SEQ ID NO. 79217, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 79217 or polypeptide SEQ ID NO. 79218, respectively, or a homolog or a fragment thereof, is increased or generated, or if the activity O-antigen chain length determinant is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 40 to 1182-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a O-antigen chain length determinant, or if the activity of the polypeptide B2027, preferably represented by SEQ ID NO. 79218, or a homolog or fragment thereof, or if the activity of a polypeptide encoded by a nucleic acid molecule comprising the nucleic acid SEQ ID NO. 79217, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 79217 or polypeptide SEQ ID NO. 79218, respectively, or a homolog or a fragment thereof, is increased or generated, or if the activity O-antigen chain length determinant is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 68 to 1185-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a GDP-mannose 4,6-dehydratase, or if the activity of the polypeptide B2053, preferably represented by SEQ ID NO. 104278, or a homolog or fragment thereof, or if the activity of a polypeptide encoded by a nucleic acid molecule comprising the nucleic acid SEQ ID NO. 104277, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 104277 or polypeptide SEQ ID NO. 104278, respectively, or a homolog or a fragment thereof, is increased or generated, or if the activity GDP-mannose 4,6-dehydratase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 22 to 50-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a transport protein, or if the activity of the polypeptide B2063, preferably represented by SEQ ID NO. 38948, or a homolog or fragment thereof, or if the activity of a polypeptide encoded by a nucleic acid molecule comprising the nucleic acid SEQ ID NO. 38947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 38947 or polypeptide SEQ ID NO. 38948, respectively, or a homolog or a fragment thereof, is increased or generated, or if the activity transport protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 35 to 778-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a transport protein, or if the activity of the polypeptide B2063, preferably represented by SEQ ID NO. 38948, or a homolog or fragment thereof, or if the activity of a polypeptide encoded by a nucleic acid molecule comprising the nucleic acid SEQ ID NO. 38947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 38947 or polypeptide SEQ ID NO. 38948, respectively, or a homolog or a fragment thereof, is increased or generated, or if the activity transport protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 62 to 415-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a Nucleoside monophosphate phosphohydrolase, or if the activity of the polypeptide B2291, preferably represented by SEQ ID NO. 104617, or a homolog or fragment thereof, or if the activity of a polypeptide encoded by a nucleic acid molecule comprising the nucleic acid SEQ ID NO. 104616, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 104616 or polypeptide SEQ ID NO. 104617, respectively, or a homolog or a fragment thereof, is increased or generated, or if the activity Nucleoside monophosphate phosphohydrolase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 21 to 41-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a transcriptional regulator, or if the activity of the polypeptide B2405, preferably represented by SEQ ID NO. 39256, or a homolog or fragment thereof, or if the activity of a polypeptide encoded by a nucleic acid molecule comprising the nucleic acid SEQ ID NO. 39255, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 39255 or polypeptide SEQ ID NO. 39256, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 54 to 74-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a transcriptional regulator, or if the activity of the polypeptide B2405, preferably represented by SEQ ID NO. 39256, or a homolog or fragment thereof, or if the activity of a polypeptide encoded by a nucleic acid molecule comprising the nucleic acid SEQ ID NO. 39255, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 39255 or polypeptide SEQ ID NO. 39256, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 24 to 74-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a b2513-protein, or if the activity of the polypeptide B2513, preferably represented by SEQ ID NO. 9168, or a homolog or fragment thereof, or if the activity of a polypeptide encoded by a nucleic acid molecule comprising the nucleic acid SEQ ID NO. 9167, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 9167 or polypeptide SEQ ID NO. 9168, respectively, or a homolog or a fragment thereof, is increased or generated, or if the activity b2513-protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 63 to 339-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a b2513-protein, or if the activity of the polypeptide B2513, preferably represented by SEQ ID NO. 9168, or a homolog or fragment thereof, or if the activity of a polypeptide encoded by a nucleic acid molecule comprising the nucleic acid SEQ ID NO. 9167, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 9167 or polypeptide SEQ ID NO. 9168, respectively, or a homolog or a fragment thereof, is increased or generated, or if the activity b2513-protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 60 to 464-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a b2513-protein, or if the activity of the polypeptide B2513, preferably represented by SEQ ID NO. 9168, or a homolog or fragment thereof, or if the activity of a polypeptide encoded by a nucleic acid molecule comprising the nucleic acid SEQ ID NO. 9167, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 9167 or polypeptide SEQ ID NO. 9168, respectively, or a homolog or a fragment thereof, is increased or generated, or if the activity b2513-protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 23 to 140-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a serine hydroxymethyltransferase, or if the activity of the polypeptide B2551, preferably represented by SEQ ID NO. 79628, or a homolog or fragment thereof, or if the activity of a polypeptide encoded by a nucleic acid molecule comprising the nucleic acid SEQ ID NO. 79627, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 79627 or polypeptide SEQ ID NO. 79628, respectively, or a homolog or a fragment thereof, is increased or generated, or if the activity serine hydroxymethyltransferase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 24 to 481-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a b2613-protein, or if the activity of the polypeptide B2613, preferably represented by SEQ ID NO. 40666, or a homolog or fragment thereof, or if the activity of a polypeptide encoded by a nucleic acid molecule comprising the nucleic acid SEQ ID NO. 40665, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 40665 or polypeptide SEQ ID NO. 40666, respectively, or a homolog or a fragment thereof, is increased or generated, or if the activity b2613-protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 24 to 29-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a transcriptional regulator, or if the activity of the polypeptide B2634, preferably represented by SEQ ID NO. 40727, or a homolog or fragment thereof, or if the activity of a polypeptide encoded by a nucleic acid molecule comprising the nucleic acid SEQ ID NO. 40726, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 40726 or polypeptide SEQ ID NO. 40727, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 34 to 118-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a transcriptional regulator, or if the activity of the polypeptide B2634, preferably represented by SEQ ID NO. 40727, or a homolog or fragment thereof, or if the activity of a polypeptide encoded by a nucleic acid molecule comprising the nucleic acid SEQ ID NO. 40726, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 40726 or polypeptide SEQ ID NO. 40727, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 52 to 124-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a b2673-protein, or if the activity of the polypeptide B2673, preferably represented by SEQ ID NO. 9245, or a homolog or fragment thereof, or if the activity of a polypeptide encoded by a nucleic acid molecule comprising the nucleic acid SEQ ID NO. 9244, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 9244 or polypeptide SEQ ID NO. 9245, respectively, or a homolog or a fragment thereof, is increased or generated, or if the activity b2673-protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 44 to 144-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a murein transglycosylase, or if the activity of the polypeptide B2701, preferably represented by SEQ ID NO. 80757, or a homolog or fragment thereof, or if the activity of a polypeptide encoded by a nucleic acid molecule comprising the nucleic acid SEQ ID NO. 80756, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 80756 or polypeptide SEQ ID NO. 80757, respectively, or a homolog or a fragment thereof, is increased or generated, or if the activity murein transglycosylase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 57 to 501-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a murein transglycosylase, or if the activity of the polypeptide B2701, preferably represented by SEQ ID NO. 80757, or a homolog or fragment thereof, or if the activity of a polypeptide encoded by a nucleic acid molecule comprising the nucleic acid SEQ ID NO. 80756, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 80756 or polypeptide SEQ ID NO. 80757, respectively, or a homolog or a fragment thereof, is increased or generated, or if the activity murein transglycosylase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 348 to 403-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a murein transglycosylase, or if the activity of the polypeptide B2701, preferably represented by SEQ ID NO. 80757, or a homolog or fragment thereof, or if the activity of a polypeptide encoded by a nucleic acid molecule comprising the nucleic acid SEQ ID NO. 80756, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 80756 or polypeptide SEQ ID NO. 80757, respectively, or a homolog or a fragment thereof, is increased or generated, or if the activity murein transglycosylase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 26 to 218-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a transcriptional regulator, or if the activity of the polypeptide B2714, preferably represented by SEQ ID NO. 40742, or a homolog or fragment thereof, or if the activity of a polypeptide encoded by a nucleic acid molecule comprising the nucleic acid SEQ ID NO. 40741, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 40741 or polypeptide SEQ ID NO. 40742, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 21 to 38-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a b2739-protein, or if the activity of the polypeptide B2739, preferably represented by SEQ ID NO. 80907, or a homolog or fragment thereof, or if the activity of a polypeptide encoded by a nucleic acid molecule comprising the nucleic acid SEQ ID NO. 80906, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 80906 or polypeptide SEQ ID NO. 80907, respectively, or a homolog or a fragment thereof, is increased or generated, or if the activity b2739-protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 51 to 542-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a b2739-protein, or if the activity of the polypeptide B2739, preferably represented by SEQ ID NO. 80907, or a homolog or fragment thereof, or if the activity of a polypeptide encoded by a nucleic acid molecule comprising the nucleic acid SEQ ID NO. 80906, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 80906 or polypeptide SEQ ID NO. 80907, respectively, or a homolog or a fragment thereof, is increased or generated, or if the activity b2739-protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 57 to 800-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a b2739-protein, or if the activity of the polypeptide B2739, preferably represented by SEQ ID NO. 80907, or a homolog or fragment thereof, or if the activity of a polypeptide encoded by a nucleic acid molecule comprising the nucleic acid SEQ ID NO. 80906, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 80906 or polypeptide SEQ ID NO. 80907, respectively, or a homolog or a fragment thereof, is increased or generated, or if the activity b2739-protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 70 to 169-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a b2812-protein, or if the activity of the polypeptide B2812, preferably represented by SEQ ID NO. 40796, or a homolog or fragment thereof, or if the activity of a polypeptide encoded by a nucleic acid molecule comprising the nucleic acid SEQ ID NO. 40795, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 40795 or polypeptide SEQ ID NO. 40796, respectively, or a homolog or a fragment thereof, is increased or generated, or if the activity b2812-protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 50 to 171-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a b2812-protein, or if the activity of the polypeptide B2812, preferably represented by SEQ ID NO. 40796, or a homolog or fragment thereof, or if the activity of a polypeptide encoded by a nucleic acid molecule comprising the nucleic acid SEQ ID NO. 40795, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 40795 or polypeptide SEQ ID NO. 40796, respectively, or a homolog or a fragment thereof, is increased or generated, or if the activity b2812-protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 59 to 203-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a transcriptional regulator, or if the activity of the polypeptide B2839, preferably represented by SEQ ID NO. 81936, or a homolog or fragment thereof, or if the activity of a polypeptide encoded by a nucleic acid molecule comprising the nucleic acid SEQ ID NO. 81935, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 81935 or polypeptide SEQ ID NO. 81936, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 35 to 190-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a transcriptional regulator, or if the activity of the polypeptide B2839, preferably represented by SEQ ID NO. 81936, or a homolog or fragment thereof, or if the activity of a polypeptide encoded by a nucleic acid molecule comprising the nucleic acid SEQ ID NO. 81935, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 81935 or polypeptide SEQ ID NO. 81936, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 118 to 154-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a transcriptional regulator, or if the activity of the polypeptide B2839, preferably represented by SEQ ID NO. 81936, or a homolog or fragment thereof, or if the activity of a polypeptide encoded by a nucleic acid molecule comprising the nucleic acid SEQ ID NO. 81935, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 81935 or polypeptide SEQ ID NO. 81936, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 30 to 94-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a ribosephosphate isomerase, constitutive, or if the activity of the polypeptide B2914, preferably represented by SEQ ID NO. 41074, or a homolog or fragment thereof, or if the activity of a polypeptide encoded by a nucleic acid molecule comprising the nucleic acid SEQ ID NO. 41073, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 41073 or polypeptide SEQ ID NO. 41074, respectively, or a homolog or a fragment thereof, is increased or generated, or if the activity ribosephosphate isomerase, constitutive is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 30 to 97-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a arginine exporter protein, or if the activity of the polypeptide B2923, preferably represented by SEQ ID NO. 9334, or a homolog or fragment thereof, or if the activity of a polypeptide encoded by a nucleic acid molecule comprising the nucleic acid SEQ ID NO. 9333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 9333 or polypeptide SEQ ID NO. 9334, respectively, or a homolog or a fragment thereof, is increased or generated, or if the activity arginine exporter protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 23 to 27-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a glycoprotease, or if the activity of the polypeptide B3064, preferably represented by SEQ ID NO. 42047, or a homolog or fragment thereof, or if the activity of a polypeptide encoded by a nucleic acid molecule comprising the nucleic acid SEQ ID NO. 42046, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 42046 or polypeptide SEQ ID NO. 42047, respectively, or a homolog or a fragment thereof, is increased or generated, or if the activity glycoprotease is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 25 to 100-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a glycoprotease, or if the activity of the polypeptide B3064, preferably represented by SEQ ID NO. 42047, or a homolog or fragment thereof, or if the activity of a polypeptide encoded by a nucleic acid molecule comprising the nucleic acid SEQ ID NO. 42046, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 42046 or polypeptide SEQ ID NO. 42047, respectively, or a homolog or a fragment thereof, is increased or generated, or if the activity glycoprotease is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 32 to 127-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a glycoprotease, or if the activity of the polypeptide B3064, preferably represented by SEQ ID NO. 42047, or a homolog or fragment thereof, or if the activity of a polypeptide encoded by a nucleic acid molecule comprising the nucleic acid SEQ ID NO. 42046, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 42046 or polypeptide SEQ ID NO. 42047, respectively, or a homolog or a fragment thereof, is increased or generated, or if the activity glycoprotease is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 32 to 82-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a b3083-protein, or if the activity of the polypeptide B3083, preferably represented by SEQ ID NO. 82425, or a homolog or fragment thereof, or if the activity of a polypeptide encoded by a nucleic acid molecule comprising the nucleic acid SEQ ID NO. 82424, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 82424 or polypeptide SEQ ID NO. 82425, respectively, or a homolog or a fragment thereof, is increased or generated, or if the activity b3083-protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 40 to 74-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a b3083-protein, or if the activity of the polypeptide B3083, preferably represented by SEQ ID NO. 82425, or a homolog or fragment thereof, or if the activity of a polypeptide encoded by a nucleic acid molecule comprising the nucleic acid SEQ ID NO. 82424, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 82424 or polypeptide SEQ ID NO. 82425, respectively, or a homolog or a fragment thereof, is increased or generated, or if the activity b3083-protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 22 to 48-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a L-serine dehydratase, or if the activity of the polypeptide B3112, preferably represented by SEQ ID NO. 104661, or a homolog or fragment thereof, or if the activity of a polypeptide encoded by a nucleic acid molecule comprising the nucleic acid SEQ ID NO. 104660, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 104660 or polypeptide SEQ ID NO. 104661, respectively, or a homolog or a fragment thereof, is increased or generated, or if the activity L-serine dehydratase is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 49 to 78-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a threonine dehydratase, or if the activity of the polypeptide B3117, preferably represented by SEQ ID NO. 104872, or a homolog or fragment thereof, or if the activity of a polypeptide encoded by a nucleic acid molecule comprising the nucleic acid SEQ ID NO. 104871, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 104871 or polypeptide SEQ ID NO. 104872, respectively, or a homolog or a fragment thereof, is increased or generated, or if the activity threonine dehydratase is increased or generated in a non-human organism, like a microorganism or a plant cell, plant or part thereof, especially with plastidic localization, whereby the respective line discloses in column 7 the fine chemical valine. For example, an increase of the valine of at least 1 percent, particularly in a range of 29 to 130-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a acetyl CoA carboxylase, or if the activity of the polypeptide B3256, preferably represented by SEQ ID NO. 9493, or a homolog or fragment thereof, or if the activity of a polypeptide encoded by a nucleic acid molecule comprising the nucleic acid SEQ ID NO. 9492, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 9492 or polypeptide SEQ ID NO. 9493, respectively, or a homolog or a fragment thereof, is increased or generated, or if the activity acetyl CoA carboxylase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 42 to 465-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a ABC transporter ATP-binding protein, or if the activity of the polypeptide B3352, preferably represented by SEQ ID NO. 82573, or a homolog or fragment thereof, or if the activity of a polypeptide encoded by a nucleic acid molecule comprising the nucleic acid SEQ ID NO. 82572, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 82572 or polypeptide SEQ ID NO. 82573, respectively, or a homolog or a fragment thereof, is increased or generated, or if the activity ABC transporter ATP-binding protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 21 to 44-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a glycogen synthase, or if the activity of the polypeptide B3429, preferably represented by SEQ ID NO. 94052, or a homolog or fragment thereof, or if the activity of a polypeptide encoded by a nucleic acid molecule comprising the nucleic acid SEQ ID NO. 94051, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 94051 or polypeptide SEQ ID NO. 94052, respectively, or a homolog or a fragment thereof, is increased or generated, or if the activity glycogen synthase is increased or generated in a non-human organism, like a microorganism or a plant cell, plant or part thereof, especially with mitochondrial localization, whereby the respective line discloses in column 7 the fine chemical isoleucine. For example, an increase of the isoleucine of at least 1 percent, particularly in a range of 73 to 242-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a glycogen synthase, or if the activity of the polypeptide B3429, preferably represented by SEQ ID NO. 94052, or a homolog or fragment thereof, or if the activity of a polypeptide encoded by a nucleic acid molecule comprising the nucleic acid SEQ ID NO. 94051, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 94051 or polypeptide SEQ ID NO. 94052, respectively, or a homolog or a fragment thereof, is increased or generated, or if the activity glycogen synthase is increased or generated in a non-human organism, like a microorganism or a plant cell, plant or part thereof, especially with mitochondrial localization, whereby the respective line discloses in column 7 the fine chemical leucine. For example, an increase of the leucine of at least 1 percent, particularly in a range of 53 to 292-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a b3490-protein, or if the activity of the polypeptide B3490, preferably represented by SEQ ID NO. 105333, or a homolog or fragment thereof, or if the activity of a polypeptide encoded by a nucleic acid molecule comprising the nucleic acid SEQ ID NO. 105332, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 105332 or polypeptide SEQ ID NO. 105333, respectively, or a homolog or a fragment thereof, is increased or generated, or if the activity b3490-protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 21 to 44-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a valine-pyruvate transaminase, or if the activity of the polypeptide B3572, preferably represented by SEQ ID NO. 10173, or a homolog or fragment thereof, or if the activity of a polypeptide encoded by a nucleic acid molecule comprising the nucleic acid SEQ ID NO. 10172, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 10172 or polypeptide SEQ ID NO. 10173, respectively, or a homolog or a fragment thereof, is increased or generated, or if the activity valine-pyruvate transaminase is increased or generated in a non-human organism, like a microorganism or a plant cell, plant or part thereof, especially with plastidic localization, whereby the respective line discloses in column 7 the fine chemical isoleucine. For example, an increase of the isoleucine of at least 1 percent, particularly in a range of 23 to 132-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a valine-pyruvate transaminase, or if the activity of the polypeptide B3572, preferably represented by SEQ ID NO. 10173, or a homolog or fragment thereof, or if the activity of a polypeptide encoded by a nucleic acid molecule comprising the nucleic acid SEQ ID NO. 10172, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 10172 or polypeptide SEQ ID NO. 10173, respectively, or a homolog or a fragment thereof, is increased or generated, or if the activity valine-pyruvate transaminase is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 32 to 115-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a phosphopantetheine adenylyltransferase, or if the activity of the polypeptide B3634, preferably represented by SEQ ID NO. 42932, or a homolog or fragment thereof, or if the activity of a polypeptide encoded by a nucleic acid molecule comprising the nucleic acid SEQ ID NO. 42931, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 42931 or polypeptide SEQ ID NO. 42932, respectively, or a homolog or a fragment thereof, is increased or generated, or if the activity phosphopantetheine adenylyltransferase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 42 to 126-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a phosphopantetheine adenylyltransferase, or if the activity of the polypeptide B3634, preferably represented by SEQ ID NO. 42932, or a homolog or fragment thereof, or if the activity of a polypeptide encoded by a nucleic acid molecule comprising the nucleic acid SEQ ID NO. 42931, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 42931 or polypeptide SEQ ID NO. 42932, respectively, or a homolog or a fragment thereof, is increased or generated, or if the activity phosphopantetheine adenylyltransferase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 94 to 289-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a phosphopantetheine adenylyltransferase, or if the activity of the polypeptide B3634, preferably represented by SEQ ID NO. 42932, or a homolog or fragment thereof, or if the activity of a polypeptide encoded by a nucleic acid molecule comprising the nucleic acid SEQ ID NO. 42931, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 42931 or polypeptide SEQ ID NO. 42932, respectively, or a homolog or a fragment thereof, is increased or generated, or if the activity phosphopantetheine adenylyltransferase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 20 to 99-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a acetolactate synthase, or if the activity of the polypeptide B3670, preferably represented by SEQ ID NO. 83630, or a homolog or fragment thereof, or if the activity of a polypeptide encoded by a nucleic acid molecule comprising the nucleic acid SEQ ID NO. 83629, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 83629 or polypeptide SEQ ID NO. 83630, respectively, or a homolog or a fragment thereof, is increased or generated, or if the activity acetolactate synthase is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 58 to 77-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a branched-chain amino-acid aminotransferase, or if the activity of the polypeptide B3770, preferably represented by SEQ ID NO. 105355, or a homolog or fragment thereof, or if the activity of a polypeptide encoded by a nucleic acid molecule comprising the nucleic acid SEQ ID NO. 105354, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 105354 or polypeptide SEQ ID NO. 105355, respectively, or a homolog or a fragment thereof, is increased or generated, or if the activity branched-chain amino-acid aminotransferase is increased or generated in a non-human organism, like a microorganism or a plant cell, plant or part thereof, especially with plastidic localization, whereby the respective line discloses in column 7 the fine chemical isoleucine. For example, an increase of the isoleucine of at least 1 percent, particularly in a range of 39 to 57-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a dihydroxyacid dehydratase, or if the activity of the polypeptide B3771, preferably represented by SEQ ID NO. 10253, or a homolog or fragment thereof, or if the activity of a polypeptide encoded by a nucleic acid molecule comprising the nucleic acid SEQ ID NO. 10252, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 10252 or polypeptide SEQ ID NO. 10253, respectively, or a homolog or a fragment thereof, is increased or generated, or if the activity dihydroxyacid dehydratase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 23 to 173-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a dihydroxyacid dehydratase, or if the activity of the polypeptide B3771, preferably represented by SEQ ID NO. 10253, or a homolog or fragment thereof, or if the activity of a polypeptide encoded by a nucleic acid molecule comprising the nucleic acid SEQ ID NO. 10252, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 10252 or polypeptide SEQ ID NO. 10253, respectively, or a homolog or a fragment thereof, is increased or generated, or if the activity dihydroxyacid dehydratase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 43 to 176-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a dihydroxyacid dehydratase, or if the activity of the polypeptide B3771, preferably represented by SEQ ID NO. 10253, or a homolog or fragment thereof, or if the activity of a polypeptide encoded by a nucleic acid molecule comprising the nucleic acid SEQ ID NO. 10252, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 10252 or polypeptide SEQ ID NO. 10253, respectively, or a homolog or a fragment thereof, is increased or generated, or if the activity dihydroxyacid dehydratase is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 42 to 49-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a dihydroxyacid dehydratase, or if the activity of the polypeptide B3771, preferably represented by SEQ ID NO. 10253, or a homolog or fragment thereof, or if the activity of a polypeptide encoded by a nucleic acid molecule comprising the nucleic acid SEQ ID NO. 10252, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 10252 or polypeptide SEQ ID NO. 10253, respectively, or a homolog or a fragment thereof, is increased or generated, or if the activity dihydroxyacid dehydratase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 37 to 128-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a ketol-acid reductoisomerase, or if the activity of the polypeptide B3774, preferably represented by SEQ ID NO. 94387, or a homolog or fragment thereof, or if the activity of a polypeptide encoded by a nucleic acid molecule comprising the nucleic acid SEQ ID NO. 94386, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 94386 or polypeptide SEQ ID NO. 94387, respectively, or a homolog or a fragment thereof, is increased or generated, or if the activity ketol-acid reductoisomerase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 55 to 61-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a ketol-acid reductoisomerase, or if the activity of the polypeptide B3774, preferably represented by SEQ ID NO. 94387, or a homolog or fragment thereof, or if the activity of a polypeptide encoded by a nucleic acid molecule comprising the nucleic acid SEQ ID NO. 94386, preferably the coding region thereof, or a homolog or fragment thereof, e.g. derived from Escherichia coli, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 94386 or polypeptide SEQ ID NO. 94387, respectively, or a homolog or a fragment thereof, is increased or generated, or if the activity ketol-acid reductoisomerase is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 38 to 127-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a DNA helicase II, or if the activity of the polypeptide B3813, preferably represented by SEQ ID NO. 43840, or a homolog or fragment thereof, or if the activity of a polypeptide encoded by a nucleic acid molecule comprising the nucleic acid SEQ ID NO. 43839, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 43839 or polypeptide SEQ ID NO. 43840, respectively, or a homolog or a fragment thereof, is increased or generated, or if the activity DNA helicase II is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 33 to 137-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a DNA helicase II, or if the activity of the polypeptide B3813, preferably represented by SEQ ID NO. 43840, or a homolog or fragment thereof, or if the activity of a polypeptide encoded by a nucleic acid molecule comprising the nucleic acid SEQ ID NO. 43839, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 43839 or polypeptide SEQ ID NO. 43840, respectively, or a homolog or a fragment thereof, is increased or generated, or if the activity DNA helicase II is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 40 to 118-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a DNA helicase II, or if the activity of the polypeptide B3813, preferably represented by SEQ ID NO. 43840, or a homolog or fragment thereof, or if the activity of a polypeptide encoded by a nucleic acid molecule comprising the nucleic acid SEQ ID NO. 43839, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 43839 or polypeptide SEQ ID NO. 43840, respectively, or a homolog or a fragment thereof, is increased or generated, or if the activity DNA helicase II is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 27 to 94-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a protoporphyrin oxidase, or if the activity of the polypeptide B3850, preferably represented by SEQ ID NO. 105603, or a homolog or fragment thereof, or if the activity of a polypeptide encoded by a nucleic acid molecule comprising the nucleic acid SEQ ID NO. 105602, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 105602 or polypeptide SEQ ID NO. 105603, respectively, or a homolog or a fragment thereof, is increased or generated, or if the activity protoporphyrin oxidase is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 181 to 667-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a superoxide dismutase, or if the activity of the polypeptide B3908, preferably represented by SEQ ID NO. 105660, or a homolog or fragment thereof, or if the activity of a polypeptide encoded by a nucleic acid molecule comprising the nucleic acid SEQ ID NO. 105659, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 105659 or polypeptide SEQ ID NO. 105660, respectively, or a homolog or a fragment thereof, is increased or generated, or if the activity superoxide dismutase is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 164 to 466-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a b3989-protein, or if the activity of the polypeptide B3989, preferably represented by SEQ ID NO. 44373, or a homolog or fragment thereof, or if the activity of a polypeptide encoded by a nucleic acid molecule comprising the nucleic acid SEQ ID NO. 44372, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 44372 or polypeptide SEQ ID NO. 44373, respectively, or a homolog or a fragment thereof, is increased or generated, or if the activity b3989-protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 34 to 1033-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a b3989-protein, or if the activity of the polypeptide B3989, preferably represented by SEQ ID NO. 44373, or a homolog or fragment thereof, or if the activity of a polypeptide encoded by a nucleic acid molecule comprising the nucleic acid SEQ ID NO. 44372, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 44372 or polypeptide SEQ ID NO. 44373, respectively, or a homolog or a fragment thereof, is increased or generated, or if the activity b3989-protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 53 to 1312-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a b3989-protein, or if the activity of the polypeptide B3989, preferably represented by SEQ ID NO. 44373, or a homolog or fragment thereof, or if the activity of a polypeptide encoded by a nucleic acid molecule comprising the nucleic acid SEQ ID NO. 44372, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 44372 or polypeptide SEQ ID NO. 44373, respectively, or a homolog or a fragment thereof, is increased or generated, or if the activity b3989-protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 23 to 332-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a b4029-protein, or if the activity of the polypeptide B4029, preferably represented by SEQ ID NO. 10741, or a homolog or fragment thereof, or if the activity of a polypeptide encoded by a nucleic acid molecule comprising the nucleic acid SEQ ID NO. 10740, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 10740 or polypeptide SEQ ID NO. 10741, respectively, or a homolog or a fragment thereof, is increased or generated, or if the activity b4029-protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 39 to 2813-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a b4029-protein, or if the activity of the polypeptide B4029, preferably represented by SEQ ID NO. 10741, or a homolog or fragment thereof, or if the activity of a polypeptide encoded by a nucleic acid molecule comprising the nucleic acid SEQ ID NO. 10740, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 10740 or polypeptide SEQ ID NO. 10741, respectively, or a homolog or a fragment thereof, is increased or generated, or if the activity b4029-protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 54 to 3551-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a b4029-protein, or if the activity of the polypeptide B4029, preferably represented by SEQ ID NO. 10741, or a homolog or fragment thereof, or if the activity of a polypeptide encoded by a nucleic acid molecule comprising the nucleic acid SEQ ID NO. 10740, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 10740 or polypeptide SEQ ID NO. 10741, respectively, or a homolog or a fragment thereof, is increased or generated, or if the activity b4029-protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 28 to 1098-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a b4050-protein, or if the activity of the polypeptide B4050, preferably represented by SEQ ID NO. 106533, or a homolog or fragment thereof, or if the activity of a polypeptide encoded by a nucleic acid molecule comprising the nucleic acid SEQ ID NO. 106532, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 106532 or polypeptide SEQ ID NO. 106533, respectively, or a homolog or a fragment thereof, is increased or generated, or if the activity b4050-protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 105 to 106-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a b4056-protein, or if the activity of the polypeptide B4056, preferably represented by SEQ ID NO. 94543, or a homolog or fragment thereof, or if the activity of a polypeptide encoded by a nucleic acid molecule comprising the nucleic acid SEQ ID NO. 94542, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 94542 or polypeptide SEQ ID NO. 94543, respectively, or a homolog or a fragment thereof, is increased or generated, or if the activity b4056-protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 39 to 51-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a b4056-protein, or if the activity of the polypeptide B4056, preferably represented by SEQ ID NO. 94543, or a homolog or fragment thereof, or if the activity of a polypeptide encoded by a nucleic acid molecule comprising the nucleic acid SEQ ID NO. 94542, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 94542 or polypeptide SEQ ID NO. 94543, respectively, or a homolog or a fragment thereof, is increased or generated, or if the activity b4056-protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 44 to 52-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a acetyltransferase, or if the activity of the polypeptide B4256, preferably represented by SEQ ID NO. 45322, or a homolog or fragment thereof, or if the activity of a polypeptide encoded by a nucleic acid molecule comprising the nucleic acid SEQ ID NO. 45321, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 45321 or polypeptide SEQ ID NO. 45322, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 51 to 240-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a acetyltransferase, or if the activity of the polypeptide B4256, preferably represented by SEQ ID NO. 45322, or a homolog or fragment thereof, or if the activity of a polypeptide encoded by a nucleic acid molecule comprising the nucleic acid SEQ ID NO. 45321, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 45321 or polypeptide SEQ ID NO. 45322, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 194 to 267-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a acetyltransferase, or if the activity of the polypeptide B4256, preferably represented by SEQ ID NO. 45322, or a homolog or fragment thereof, or if the activity of a polypeptide encoded by a nucleic acid molecule comprising the nucleic acid SEQ ID NO. 45321, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 45321 or polypeptide SEQ ID NO. 45322, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 53 to 137-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a b4263-protein, or if the activity of the polypeptide B4263, preferably represented by SEQ ID NO. 106539, or a homolog or fragment thereof, or if the activity of a polypeptide encoded by a nucleic acid molecule comprising the nucleic acid SEQ ID NO. 106538, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 106538 or polypeptide SEQ ID NO. 106539, respectively, or a homolog or a fragment thereof, is increased or generated, or if the activity b4263-protein is increased or generated in a non-human organism, like a microorganism or a plant cell, plant or part thereof, especially with plastidic localization, whereby the respective line discloses in column 7 the fine chemical valine. For example, an increase of the valine of at least 1 percent, particularly in a range of 23 to 38-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a gluconate transport system permease 3, or if the activity of the polypeptide B4321, preferably represented by SEQ ID NO. 45395, or a homolog or fragment thereof, or if the activity of a polypeptide encoded by a nucleic acid molecule comprising the nucleic acid SEQ ID NO. 45394, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 45394 or polypeptide SEQ ID NO. 45395, respectively, or a homolog or a fragment thereof, is increased or generated, or if the activity gluconate transport system permease 3 is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 43 to 228-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a gluconate transport system permease 3, or if the activity of the polypeptide B4321, preferably represented by SEQ ID NO. 45395, or a homolog or fragment thereof, or if the activity of a polypeptide encoded by a nucleic acid molecule comprising the nucleic acid SEQ ID NO. 45394, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 45394 or polypeptide SEQ ID NO. 45395, respectively, or a homolog or a fragment thereof, is increased or generated, or if the activity gluconate transport system permease 3 is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 113 to 284-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a gluconate transport system permease 3, or if the activity of the polypeptide B4321, preferably represented by SEQ ID NO. 45395, or a homolog or fragment thereof, or if the activity of a polypeptide encoded by a nucleic acid molecule comprising the nucleic acid SEQ ID NO. 45394, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 45394 or polypeptide SEQ ID NO. 45395, respectively, or a homolog or a fragment thereof, is increased or generated, or if the activity gluconate transport system permease 3 is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 20 to 85-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a purinenucleoside phosphorylase, or if the activity of the polypeptide B4384, preferably represented by SEQ ID NO. 45557, or a homolog or fragment thereof, or if the activity of a polypeptide encoded by a nucleic acid molecule comprising the nucleic acid SEQ ID NO. 45556, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 45556 or polypeptide SEQ ID NO. 45557, respectively, or a homolog or a fragment thereof, is increased or generated, or if the activity purine-nucleoside phosphorylase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 64 to 169-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a thioredoxin, or if the activity of the polypeptide C_pp004096192r, preferably represented by SEQ ID NO. 45758, or a homolog or fragment thereof, or if the activity of a polypeptide encoded by a nucleic acid molecule comprising the nucleic acid SEQ ID NO. 45757, preferably the coding region thereof, or a homolog or fragment thereof, e.g. derived from Physcomitrella patens, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 45757 or polypeptide SEQ ID NO. 45758, respectively, or a homolog or a fragment thereof, is increased or generated, or if the activity thioredoxin is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 85 to 176-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a thioredoxin, or if the activity of the polypeptide C_pp004096192r, preferably represented by SEQ ID NO. 45758, or a homolog or fragment thereof, or if the activity of a polypeptide encoded by a nucleic acid molecule comprising the nucleic acid SEQ ID NO. 45757, preferably the coding region thereof, or a homolog or fragment thereof, e.g. derived from Physcomitrella patens, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 45757 or polypeptide SEQ ID NO. 45758, respectively, or a homolog or a fragment thereof, is increased or generated, or if the activity thioredoxin is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 65 to 147-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a thioredoxin, or if the activity of the polypeptide C_pp004096192r, preferably represented by SEQ ID NO. 45758, or a homolog or fragment thereof, or if the activity of a polypeptide encoded by a nucleic acid molecule comprising the nucleic acid SEQ ID NO. 45757, preferably the coding region thereof, or a homolog or fragment thereof, e.g. derived from Physcomitrella patens, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 45757 or polypeptide SEQ ID NO. 45758, respectively, or a homolog or a fragment thereof, is increased or generated, or if the activity thioredoxin is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 21 to 74-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a GM02LC11114-protein, or if the activity of the polypeptide GM02LC11114, preferably represented by SEQ ID NO. 45796, or a homolog or fragment thereof, or if the activity of a polypeptide encoded by a nucleic acid molecule comprising the nucleic acid SEQ ID NO. 45795, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 45795 or polypeptide SEQ ID NO. 45796, respectively, or a homolog or a fragment thereof, is increased or generated, or if the activity GM02LC11114-protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 64 to 105-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a GM02LC11114-protein, or if the activity of the polypeptide GM02LC11114, preferably represented by SEQ ID NO. 45796, or a homolog or fragment thereof, or if the activity of a polypeptide encoded by a nucleic acid molecule comprising the nucleic acid SEQ ID NO. 45795, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 45795 or polypeptide SEQ ID NO. 45796, respectively, or a homolog or a fragment thereof, is increased or generated, or if the activity GM02LC11114-protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 62 to 113-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a glutaredoxin, or if the activity of the polypeptide GM02LC12622, preferably represented by SEQ ID NO. 10812, or a homolog or fragment thereof, or if the activity of a polypeptide encoded by a nucleic acid molecule comprising the nucleic acid SEQ ID NO. 10811, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 10811 or polypeptide SEQ ID NO. 10812, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 43 to 472-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a glutaredoxin, or if the activity of the polypeptide GM02LC12622, preferably represented by SEQ ID NO. 10812, or a homolog or fragment thereof, or if the activity of a polypeptide encoded by a nucleic acid molecule comprising the nucleic acid SEQ ID NO. 10811, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 10811 or polypeptide SEQ ID NO. 10812, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 63 to 562-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a glutaredoxin, or if the activity of the polypeptide GM02LC12622, preferably represented by SEQ ID NO. 10812, or a homolog or fragment thereof, or if the activity of a polypeptide encoded by a nucleic acid molecule comprising the nucleic acid SEQ ID NO. 10811, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 10811 or polypeptide SEQ ID NO. 10812, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 24 to 155-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a histone H2A, or if the activity of the polypeptide GM02LC15313, preferably represented by SEQ ID NO. 45898, or a homolog or fragment thereof, or if the activity of a polypeptide encoded by a nucleic acid molecule comprising the nucleic acid SEQ ID NO. 45897, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 45897 or polypeptide SEQ ID NO. 45898, respectively, or a homolog or a fragment thereof, is increased or generated, or if the activity histone H2A is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 53 to 172-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a histone H2A, or if the activity of the polypeptide GM02LC15313, preferably represented by SEQ ID NO. 45898, or a homolog or fragment thereof, or if the activity of a polypeptide encoded by a nucleic acid molecule comprising the nucleic acid SEQ ID NO. 45897, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 45897 or polypeptide SEQ ID NO. 45898, respectively, or a homolog or a fragment thereof, is increased or generated, or if the activity histone H2A is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 80 to 144-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a histone H2A, or if the activity of the polypeptide GM02LC15313, preferably represented by SEQ ID NO. 45898, or a homolog or fragment thereof, or if the activity of a polypeptide encoded by a nucleic acid molecule comprising the nucleic acid SEQ ID NO. 45897, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 45897 or polypeptide SEQ ID NO. 45898, respectively, or a homolog or a fragment thereof, is increased or generated, or if the activity histone H2A is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 30 to 99-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a vacuolar ATPase subunit, or if the activity of the polypeptide GM02LC17384, preferably represented by SEQ ID NO. 106689, or a homolog or fragment thereof, or if the activity of a polypeptide encoded by a nucleic acid molecule comprising the nucleic acid SEQ ID NO. 106688, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 106688 or polypeptide SEQ ID NO. 106689, respectively, or a homolog or a fragment thereof, is increased or generated, or if the activity vacuolar ATPase subunit is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 21 to 63-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a RNA binding protein, or if the activity of the polypeptide GM02LC17556, preferably represented by SEQ ID NO. 46516, or a homolog or fragment thereof, or if the activity of a polypeptide encoded by a nucleic acid molecule comprising the nucleic acid SEQ ID NO. 46515, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 46515 or polypeptide SEQ ID NO. 46516, respectively, or a homolog or a fragment thereof, is increased or generated, or if the activity RNA binding protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 51 to 366-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a RNA binding protein, or if the activity of the polypeptide GM02LC17556, preferably represented by SEQ ID NO. 46516, or a homolog or fragment thereof, or if the activity of a polypeptide encoded by a nucleic acid molecule comprising the nucleic acid SEQ ID NO. 46515, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 46515 or polypeptide SEQ ID NO. 46516, respectively, or a homolog or a fragment thereof, is increased or generated, or if the activity RNA binding protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 55 to 406-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a RNA binding protein, or if the activity of the polypeptide GM02LC17556, preferably represented by SEQ ID NO. 46516, or a homolog or fragment thereof, or if the activity of a polypeptide encoded by a nucleic acid molecule comprising the nucleic acid SEQ ID NO. 46515, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 46515 or polypeptide SEQ ID NO. 46516, respectively, or a homolog or a fragment thereof, is increased or generated, or if the activity RNA binding protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 21 to 90-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a transcription factor, or if the activity of the polypeptide GM02LC21368, preferably represented by SEQ ID NO. 84199, or a homolog or fragment thereof, or if the activity of a polypeptide encoded by a nucleic acid molecule comprising the nucleic acid SEQ ID NO. 84198, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 84198 or polypeptide SEQ ID NO. 84199, respectively, or a homolog or a fragment thereof, is increased or generated, or if the activity transcription factor is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 54 to 890-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a transcription factor, or if the activity of the polypeptide GM02LC21368, preferably represented by SEQ ID NO. 84199, or a homolog or fragment thereof, or if the activity of a polypeptide encoded by a nucleic acid molecule comprising the nucleic acid SEQ ID NO. 84198, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 84198 or polypeptide SEQ ID NO. 84199, respectively, or a homolog or a fragment thereof, is increased or generated, or if the activity transcription factor is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 100 to 811-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a transcription factor, or if the activity of the polypeptide GM02LC21368, preferably represented by SEQ ID NO. 84199, or a homolog or fragment thereof, or if the activity of a polypeptide encoded by a nucleic acid molecule comprising the nucleic acid SEQ ID NO. 84198, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 84198 or polypeptide SEQ ID NO. 84199, respectively, or a homolog or a fragment thereof, is increased or generated, or if the activity transcription factor is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 21 to 230-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a cyclin D, or if the activity of the polypeptide GM02LC44512, preferably represented by SEQ ID NO. 46851, or a homolog or fragment thereof, or if the activity of a polypeptide encoded by a nucleic acid molecule comprising the nucleic acid SEQ ID NO. 46850, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 46850 or polypeptide SEQ ID NO. 46851, respectively, or a homolog or a fragment thereof, is increased or generated, or if the activity cyclin D is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 40 to 128-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a cyclin D, or if the activity of the polypeptide GM02LC44512, preferably represented by SEQ ID NO. 46851, or a homolog or fragment thereof, or if the activity of a polypeptide encoded by a nucleic acid molecule comprising the nucleic acid SEQ ID NO. 46850, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 46850 or polypeptide SEQ ID NO. 46851, respectively, or a homolog or a fragment thereof, is increased or generated, or if the activity cyclin D is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 82 to 122-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a cyclin D, or if the activity of the polypeptide GM02LC44512, preferably represented by SEQ ID NO. 46851, or a homolog or fragment thereof, or if the activity of a polypeptide encoded by a nucleic acid molecule comprising the nucleic acid SEQ ID NO. 46850, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 46850 or polypeptide SEQ ID NO. 46851, respectively, or a homolog or a fragment thereof, is increased or generated, or if the activity cyclin D is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 22 to 86-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a GM02LC5744-protein, or if the activity of the polypeptide GM02LC5744, preferably represented by SEQ ID NO. 47077, or a homolog or fragment thereof, or if the activity of a polypeptide encoded by a nucleic acid molecule comprising the nucleic acid SEQ ID NO. 47076, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 47076 or polypeptide SEQ ID NO. 47077, respectively, or a homolog or a fragment thereof, is increased or generated, or if the activity GM02LC5744-protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 53 to 754-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a agmatinase, or if the activity of the polypeptide Sll0228, preferably represented by SEQ ID NO. 84266, or a homolog or fragment thereof, or if the activity of a polypeptide encoded by a nucleic acid molecule comprising the nucleic acid SEQ ID NO. 84265, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 84265 or polypeptide SEQ ID NO. 84266, respectively, or a homolog or a fragment thereof, is increased or generated, or if the activity agmatinase is increased or generated in a non-human organism, like a microorganism or a plant cell, plant or part thereof, especially with plastidic localization, whereby the respective line discloses in column 7 the fine chemical valine. For example, an increase of the valine of at least 1 percent, particularly in a range of 21 to 33-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a ABC transporter ATP binding component, or if the activity of the polypeptide Sll0240, preferably represented by SEQ ID NO. 84442, or a homolog or fragment thereof, or if the activity of a polypeptide encoded by a nucleic acid molecule comprising the nucleic acid SEQ ID NO. 84441, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 84441 or polypeptide SEQ ID NO. 84442, respectively, or a homolog or a fragment thereof, is increased or generated, or if the activity ABC transporter ATP binding component is increased or generated in a non-human organism, like a microorganism or a plant cell, plant or part thereof, especially with plastidic localization, whereby the respective line discloses in column 7 the fine chemical valine. For example, an increase of the valine of at least 1 percent, particularly in a range of 24 to 32-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a flavodoxin, or if the activity of the polypeptide Sll0248, preferably represented by SEQ ID NO. 49144, or a homolog or fragment thereof, or if the activity of a polypeptide encoded by a nucleic acid molecule comprising the nucleic acid SEQ ID NO. 49143, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 49143 or polypeptide SEQ ID NO. 49144, respectively, or a homolog or a fragment thereof, is increased or generated, or if the activity flavodoxin is increased or generated in a non-human organism, like a microorganism or a plant cell, plant or part thereof, especially with plastidic localization, whereby the respective line discloses in column 7 the fine chemical isoleucine. For example, an increase of the isoleucine of at least 1 percent, particularly in a range of 53 to 313-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a flavodoxin, or if the activity of the polypeptide Sll0248, preferably represented by SEQ ID NO. 49144, or a homolog or fragment thereof, or if the activity of a polypeptide encoded by a nucleic acid molecule comprising the nucleic acid SEQ ID NO. 49143, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 49143 or polypeptide SEQ ID NO. 49144, respectively, or a homolog or a fragment thereof, is increased or generated, or if the activity flavodoxin is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 70 to 203-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a polyphosphate kinase, or if the activity of the polypeptide Sll0290, preferably represented by SEQ ID NO. 49829, or a homolog or fragment thereof, or if the activity of a polypeptide encoded by a nucleic acid molecule comprising the nucleic acid SEQ ID NO. 49828, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 49828 or polypeptide SEQ ID NO. 49829, respectively, or a homolog or a fragment thereof, is increased or generated, or if the activity polyphosphate kinase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 35 to 238-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a polyphosphate kinase, or if the activity of the polypeptide Sll0290, preferably represented by SEQ ID NO. 49829, or a homolog or fragment thereof, or if the activity of a polypeptide encoded by a nucleic acid molecule comprising the nucleic acid SEQ ID NO. 49828, preferably the coding region thereof, or a homolog or fragment thereof, e.g. derived from Synechocystis sp., is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 49828 or polypeptide SEQ ID NO. 49829, respectively, or a homolog or a fragment thereof, is increased or generated, or if the activity polyphosphate kinase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 70 to 192-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a polyphosphate kinase, or if the activity of the polypeptide Sll0290, preferably represented by SEQ ID NO. 49829, or a homolog or fragment thereof, or if the activity of a polypeptide encoded by a nucleic acid molecule comprising the nucleic acid SEQ ID NO. 49828, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 49828 or polypeptide SEQ ID NO. 49829, respectively, or a homolog or a fragment thereof, is increased or generated, or if the activity polyphosphate kinase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 22 to 98-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a bifunctional protein (phosphoribosyltransferase and regulatory protein), or if the activity of the polypeptide Sll0368, preferably represented by SEQ ID NO. 106921, or a homolog or fragment thereof, or if the activity of a polypeptide encoded by a nucleic acid molecule comprising the nucleic acid SEQ ID NO. 106920, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 106920 or polypeptide SEQ ID NO. 106921, respectively, or a homolog or a fragment thereof, is increased or generated, or if the activity bifunctional protein (phosphoribosyltransferase and regulatory protein) is increased or generated in a non-human organism, like a microorganism or a plant cell, plant or part thereof, especially with mitochondrial localization, whereby the respective line discloses in column 7 the fine chemical valine. For example, an increase of the valine of at least 1 percent, particularly in a range of 24 to 39-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a urease subunit, or if the activity of the polypeptide Sll0420, preferably represented by SEQ ID NO. 50105, or a homolog or fragment thereof, or if the activity of a polypeptide encoded by a nucleic acid molecule comprising the nucleic acid SEQ ID NO. 50104, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 50104 or polypeptide SEQ ID NO. 50105, respectively, or a homolog or a fragment thereof, is increased or generated, or if the activity urease subunit is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 41 to 69-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a NAD(P)H-quinone oxidoreductase subunit, or if the activity of the polypeptide Sll0521, preferably represented by SEQ ID NO. 50340, or a homolog or fragment thereof, or if the activity of a polypeptide encoded by a nucleic acid molecule comprising the nucleic acid SEQ ID NO. 50339, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 50339 or polypeptide SEQ ID NO. 50340, respectively, or a homolog or a fragment thereof, is increased or generated, or if the activity NAD(P)H-quinone oxidoreductase subunit is increased or generated in a non-human organism, like a microorganism or a plant cell, plant or part thereof, especially with plastidic localization, whereby the respective line discloses in column 7 the fine chemical valine. For example, an increase of the valine of at least 1 percent, particularly in a range of 21 to 56-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a oxireductase, or if the activity of the polypeptide Sll0816, preferably represented by SEQ ID NO. 51199, or a homolog or fragment thereof, or if the activity of a polypeptide encoded by a nucleic acid molecule comprising the nucleic acid SEQ ID NO. 51198, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 51198 or polypeptide SEQ ID NO. 51199, respectively, or a homolog or a fragment thereof, is increased or generated, or if the activity oxireductase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 34 to 55-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a binding-protein-dependent transport systems inner membrane component, or if the activity of the polypeptide Sll0833, preferably represented by SEQ ID NO. 101708, or a homolog or fragment thereof, or if the activity of a polypeptide encoded by a nucleic acid molecule comprising the nucleic acid SEQ ID NO. 101707, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 101707 or polypeptide SEQ ID NO. 101708, respectively, or a homolog or a fragment thereof, is increased or generated, or if the activity binding-protein-dependent transport systems inner membrane component is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 39 to 124-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a sll1032-protein, or if the activity of the polypeptide Sll1032, preferably represented by SEQ ID NO. 85724, or a homolog or fragment thereof, or if the activity of a polypeptide encoded by a nucleic acid molecule comprising the nucleic acid SEQ ID NO. 85723, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 85723 or polypeptide SEQ ID NO. 85724, respectively, or a homolog or a fragment thereof, is increased or generated, or if the activity sll1032-protein is increased or generated in a non-human organism, like a microorganism or a plant cell, plant or part thereof, especially with mitochondrial localization, whereby the respective line discloses in column 7 the fine chemical valine. For example, an increase of the valine of at least 1 percent, particularly in a range of 19 to 29-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a glycogen (starch) synthase, or if the activity of the polypeptide Sll1393, preferably represented by SEQ ID NO. 53457, or a homolog or fragment thereof, or if the activity of a polypeptide encoded by a nucleic acid molecule comprising the nucleic acid SEQ ID NO. 53456, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 53456 or polypeptide SEQ ID NO. 53457, respectively, or a homolog or a fragment thereof, is increased or generated, or if the activity glycogen (starch) synthase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 40 to 192-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a glycogen (starch) synthase, or if the activity of the polypeptide Sll1393, preferably represented by SEQ ID NO. 53457, or a homolog or fragment thereof, or if the activity of a polypeptide encoded by a nucleic acid molecule comprising the nucleic acid SEQ ID NO. 53456, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 53456 or polypeptide SEQ ID NO. 53457, respectively, or a homolog or a fragment thereof, is increased or generated, or if the activity glycogen (starch) synthase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 99 to 132-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a glycogen (starch) synthase, or if the activity of the polypeptide Sll1393, preferably represented by SEQ ID NO. 53457, or a homolog or fragment thereof, or if the activity of a polypeptide encoded by a nucleic acid molecule comprising the nucleic acid SEQ ID NO. 53456, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 53456 or polypeptide SEQ ID NO. 53457, respectively, or a homolog or a fragment thereof, is increased or generated, or if the activity glycogen (starch) synthase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 19 to 121-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a sll1455-protein, or if the activity of the polypeptide Sll1455, preferably represented by SEQ ID NO. 107101, or a homolog or fragment thereof, or if the activity of a polypeptide encoded by a nucleic acid molecule comprising the nucleic acid SEQ ID NO. 107100, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 107100 or polypeptide SEQ ID NO. 107101, respectively, or a homolog or a fragment thereof, is increased or generated, or if the activity sll1455-protein is increased or generated in a non-human organism, like a microorganism or a plant cell, plant or part thereof, especially with plastidic localization, whereby the respective line discloses in column 7 the fine chemical valine. For example, an increase of the valine of at least 1 percent, particularly in a range of 21 to 36-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a carbamoylphosphate synthase subunit, or if the activity of the polypeptide Sll1498, preferably represented by SEQ ID NO. 95048, or a homolog or fragment thereof, or if the activity of a polypeptide encoded by a nucleic acid molecule comprising the nucleic acid SEQ ID NO. 95047, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 95047 or polypeptide SEQ ID NO. 95048, respectively, or a homolog or a fragment thereof, is increased or generated, or if the activity carbamoyl-phosphate synthase subunit is increased or generated in a non-human organism, like a microorganism or a plant cell, plant or part thereof, especially with plastidic localization, whereby the respective line discloses in column 7 the fine chemical isoleucine. For example, an increase of the isoleucine of at least 1 percent, particularly in a range of 45 to 71-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a carbamoyl-phosphate synthase subunit, or if the activity of the polypeptide Sll1498, preferably represented by SEQ ID NO. 95048, or a homolog or fragment thereof, or if the activity of a polypeptide encoded by a nucleic acid molecule comprising the nucleic acid SEQ ID NO. 95047, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 95047 or polypeptide SEQ ID NO. 95048, respectively, or a homolog or a fragment thereof, is increased or generated, or if the activity carbamoyl-phosphate synthase subunit is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 53 to 90-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a sll1761-protein, or if the activity of the polypeptide Sll1761, preferably represented by SEQ ID NO. 55380, or a homolog or fragment thereof, or if the activity of a polypeptide encoded by a nucleic acid molecule comprising the nucleic acid SEQ ID NO. 55379, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 55379 or polypeptide SEQ ID NO. 55380, respectively, or a homolog or a fragment thereof, is increased or generated, or if the activity sll1761-protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 56 to 276-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a sll1761-protein, or if the activity of the polypeptide Sll1761, preferably represented by SEQ ID NO. 55380, or a homolog or fragment thereof, or if the activity of a polypeptide encoded by a nucleic acid molecule comprising the nucleic acid SEQ ID NO. 55379, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 55379 or polypeptide SEQ ID NO. 55380, respectively, or a homolog or a fragment thereof, is increased or generated, or if the activity sll1761-protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 67 to 245-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a adenylate kinase, or if the activity of the polypeptide Sll1815, preferably represented by SEQ ID NO. 85809, or a homolog or fragment thereof, or if the activity of a polypeptide encoded by a nucleic acid molecule comprising the nucleic acid SEQ ID NO. 85808, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 85808 or polypeptide SEQ ID NO. 85809, respectively, or a homolog or a fragment thereof, is increased or generated, or if the activity adenylate kinase is increased or generated in a non-human organism, like a microorganism or a plant cell, plant or part thereof, especially with mitochondrial localization, whereby the respective line discloses in column 7 the fine chemical isoleucine. For example, an increase of the isoleucine of at least 1 percent, particularly in a range of 103 to 134-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a adenylate kinase, or if the activity of the polypeptide Sll1815, preferably represented by SEQ ID NO. 85809, or a homolog or fragment thereof, or if the activity of a polypeptide encoded by a nucleic acid molecule comprising the nucleic acid SEQ ID NO. 85808, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 85808 or polypeptide SEQ ID NO. 85809, respectively, or a homolog or a fragment thereof, is increased or generated, or if the activity adenylate kinase is increased or generated in a non-human organism, like a microorganism or a plant cell, plant or part thereof, especially with mitochondrial localization, whereby the respective line discloses in column 7 the fine chemical leucine. For example, an increase of the leucine of at least 1 percent, particularly in a range of 102 to 148-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a adenylate kinase, or if the activity of the polypeptide Sll1815, preferably represented by SEQ ID NO. 85809, or a homolog or fragment thereof, or if the activity of a polypeptide encoded by a nucleic acid molecule comprising the nucleic acid SEQ ID NO. 85808, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 85808 or polypeptide SEQ ID NO. 85809, respectively, or a homolog or a fragment thereof, is increased or generated, or if the activity adenylate kinase is increased or generated in a non-human organism, like a microorganism or a plant cell, plant or part thereof, especially with mitochondrial localization, whereby the respective line discloses in column 7 the fine chemical valine. For example, an increase of the valine of at least 1 percent, particularly in a range of 21 to 138-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a acyl transferase, or if the activity of the polypeptide Sll1848, preferably represented by SEQ ID NO. 86448, or a homolog or fragment thereof, or if the activity of a polypeptide encoded by a nucleic acid molecule comprising the nucleic acid SEQ ID NO. 86447, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 86447 or polypeptide SEQ ID NO. 86448, respectively, or a homolog or a fragment thereof, is increased or generated, or if the activity acyl transferase is increased or generated in a non-human organism, like a microorganism or a plant cell, plant or part thereof, especially with plastidic localization, whereby the respective line discloses in column 7 the fine chemical isoleucine. For example, an increase of the isoleucine of at least 1 percent, particularly in a range of 140 to 570-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a ferredoxin, or if the activity of the polypeptide Slr0150, preferably represented by SEQ ID NO. 95528, or a homolog or fragment thereof, or if the activity of a polypeptide encoded by a nucleic acid molecule comprising the nucleic acid SEQ ID NO. 95527, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 95527 or polypeptide SEQ ID NO. 95528, respectively, or a homolog or a fragment thereof, is increased or generated, or if the activity ferredoxin is increased or generated in a non-human organism, like a microorganism or a plant cell, plant or part thereof, especially with mitochondrial localization, whereby the respective line discloses in column 7 the fine chemical valine. For example, an increase of the valine of at least 1 percent, particularly in a range of 23 to 47-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a precorrin methylase, or if the activity of the polypeptide Slr0239, preferably represented by SEQ ID NO. 86541, or a homolog or fragment thereof, or if the activity of a polypeptide encoded by a nucleic acid molecule comprising the nucleic acid SEQ ID NO. 86540, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 86540 or polypeptide SEQ ID NO. 86541, respectively, or a homolog or a fragment thereof, is increased or generated, or if the activity precorrin methylase is increased or generated in a non-human organism, like a microorganism or a plant cell, plant or part thereof, especially with mitochondrial localization, whereby the respective line discloses in column 7 the fine chemical isoleucine. For example, an increase of the isoleucine of at least 1 percent, particularly in a range of 33 to 447-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a precorrin methylase, or if the activity of the polypeptide Slr0239, preferably represented by SEQ ID NO. 86541, or a homolog or fragment thereof, or if the activity of a polypeptide encoded by a nucleic acid molecule comprising the nucleic acid SEQ ID NO. 86540, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 86540 or polypeptide SEQ ID NO. 86541, respectively, or a homolog or a fragment thereof, is increased or generated, or if the activity precorrin methylase is increased or generated in a non-human organism, like a microorganism or a plant cell, plant or part thereof, especially with mitochondrial localization, whereby the respective line discloses in column 7 the fine chemical leucine. For example, an increase of the leucine of at least 1 percent, particularly in a range of 212 to 603-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a precorrin methylase, or if the activity of the polypeptide 51r0239, preferably represented by SEQ ID NO. 86541, or a homolog or fragment thereof, or if the activity of a polypeptide encoded by a nucleic acid molecule comprising the nucleic acid SEQ ID NO. 86540, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 86540 or polypeptide SEQ ID NO. 86541, respectively, or a homolog or a fragment thereof, is increased or generated, or if the activity precorrin methylase is increased or generated in a non-human organism, like a microorganism or a plant cell, plant or part thereof, especially with mitochondrial localization, whereby the respective line discloses in column 7 the fine chemical valine. For example, an increase of the valine of at least 1 percent, particularly in a range of 25 to 185-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a malate dehydrogenase, or if the activity of the polypeptide Slr0721, preferably represented by SEQ ID NO. 87398, or a homolog or fragment thereof, or if the activity of a polypeptide encoded by a nucleic acid molecule comprising the nucleic acid SEQ ID NO. 87397, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 87397 or polypeptide SEQ ID NO. 87398, respectively, or a homolog or a fragment thereof, is increased or generated, or if the activity malate dehydrogenase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 49 to 129-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a malate dehydrogenase, or if the activity of the polypeptide Slr0721, preferably represented by SEQ ID NO. 87398, or a homolog or fragment thereof, or if the activity of a polypeptide encoded by a nucleic acid molecule comprising the nucleic acid SEQ ID NO. 87397, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 87397 or polypeptide SEQ ID NO. 87398, respectively, or a homolog or a fragment thereof, is increased or generated, or if the activity malate dehydrogenase is increased or generated in a non-human organism, like a microorganism or a plant cell, plant or part thereof, especially with plastidic localization, whereby the respective line discloses in column 7 the fine chemical isoleucine. For example, an increase of the isoleucine of at least 1 percent, particularly in a range of 38 to 175-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a malate dehydrogenase, or if the activity of the polypeptide Slr0721, preferably represented by SEQ ID NO. 87398, or a homolog or fragment thereof, or if the activity of a polypeptide encoded by a nucleic acid molecule comprising the nucleic acid SEQ ID NO. 87397, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 87397 or polypeptide SEQ ID NO. 87398, respectively, or a homolog or a fragment thereof, is increased or generated, or if the activity malate dehydrogenase is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 98 to 267-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a malate dehydrogenase, or if the activity of the polypeptide Slr0721, preferably represented by SEQ ID NO. 87398, or a homolog or fragment thereof, or if the activity of a polypeptide encoded by a nucleic acid molecule comprising the nucleic acid SEQ ID NO. 87397, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 87397 or polypeptide SEQ ID NO. 87398, respectively, or a homolog or a fragment thereof, is increased or generated, or if the activity malate dehydrogenase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 79 to 191-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a malate dehydrogenase, or if the activity of the polypeptide Slr0721, preferably represented by SEQ ID NO. 87398, or a homolog or fragment thereof, or if the activity of a polypeptide encoded by a nucleic acid molecule comprising the nucleic acid SEQ ID NO. 87397, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 87397 or polypeptide SEQ ID NO. 87398, respectively, or a homolog or a fragment thereof, is increased or generated, or if the activity malate dehydrogenase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 20 to 60-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a malate dehydrogenase, or if the activity of the polypeptide Slr0721, preferably represented by SEQ ID NO. 87398, or a homolog or fragment thereof, or if the activity of a polypeptide encoded by a nucleic acid molecule comprising the nucleic acid SEQ ID NO. 87397, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 87397 or polypeptide SEQ ID NO. 87398, respectively, or a homolog or a fragment thereof, is increased or generated, or if the activity malate dehydrogenase is increased or generated in a non-human organism, like a microorganism or a plant cell, plant or part thereof, especially with plastidic localization, whereby the respective line discloses in column 7 the fine chemical valine. For example, an increase of the valine of at least 1 percent, particularly in a range of 21 to 98-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a geranylgeranyl pyrophosphate synthase, or if the activity of the polypeptide Slr0739, preferably represented by SEQ ID NO. 57236, or a homolog or fragment thereof, or if the activity of a polypeptide encoded by a nucleic acid molecule comprising the nucleic acid SEQ ID NO. 57235, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 57235 or polypeptide SEQ ID NO. 57236, respectively, or a homolog or a fragment thereof, is increased or generated, or if the activity geranylgeranyl pyrophosphate synthase is increased or generated in a non-human organism, like a microorganism or a plant cell, plant or part thereof, especially with plastidic localization, whereby the respective line discloses in column 7 the fine chemical isoleucine. For example, an increase of the isoleucine of at least 1 percent, particularly in a range of 37 to 120-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a geranylgeranyl pyrophosphate synthase, or if the activity of the polypeptide Slr0739, preferably represented by SEQ ID NO. 57236, or a homolog or fragment thereof, or if the activity of a polypeptide encoded by a nucleic acid molecule comprising the nucleic acid SEQ ID NO. 57235, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 57235 or polypeptide SEQ ID NO. 57236, respectively, or a homolog or a fragment thereof, is increased or generated, or if the activity geranylgeranyl pyrophosphate synthase is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 60 to 128 -percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a geranylgeranyl pyrophosphate synthase, or if the activity of the polypeptide Slr0739, preferably represented by SEQ ID NO. 57236, or a homolog or fragment thereof, or if the activity of a polypeptide encoded by a nucleic acid molecule comprising the nucleic acid SEQ ID NO. 57235, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 57235 or polypeptide SEQ ID NO. 57236, respectively, or a homolog or a fragment thereof, is increased or generated, or if the activity geranylgeranyl pyrophosphate synthase is increased or generated in a non-human organism, like a microorganism or a plant cell, plant or part thereof, especially with plastidic localization, whereby the respective line discloses in column 7 the fine chemical valine. For example, an increase of the valine of at least 1 percent, particularly in a range of 22 to 91-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a iron(III) dicitrate-binding protein, or if the activity of the polypeptide Slr1492, preferably represented by SEQ ID NO. 58669, or a homolog or fragment thereof, or if the activity of a polypeptide encoded by a nucleic acid molecule comprising the nucleic acid SEQ ID NO. 58668, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 58668 or polypeptide SEQ ID NO. 58669, respectively, or a homolog or a fragment thereof, is increased or generated, or if the activity iron(III) dicitrate-binding protein is increased or generated in a non-human organism, like a microorganism or a plant cell, plant or part thereof, especially with mitochondrial localization, whereby the respective line discloses in column 7 the fine chemical isoleucine. For example, an increase of the isoleucine of at least 1 percent, particularly in a range of 27 to 42-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a Na+/K+ transporter, or if the activity of the polypeptide Slr1509, preferably represented by SEQ ID NO. 88159, or a homolog or fragment thereof, or if the activity of a polypeptide encoded by a nucleic acid molecule comprising the nucleic acid SEQ ID NO. 88158, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 88158 or polypeptide SEQ ID NO. 88159, respectively, or a homolog or a fragment thereof, is increased or generated, or if the activity Na+/K+ transporter is increased or generated in a non-human organism, like a microorganism or a plant cell, plant or part thereof, especially with mitochondrial localization, whereby the respective line discloses in column 7 the fine chemical isoleucine. For example, an increase of the isoleucine of at least 1 percent, particularly in a range of 45 to 122-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a Na+/K+ transporter, or if the activity of the polypeptide Slr1509, preferably represented by SEQ ID NO. 88159, or a homolog or fragment thereof, or if the activity of a polypeptide encoded by a nucleic acid molecule comprising the nucleic acid SEQ ID NO. 88158, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 88158 or polypeptide SEQ ID NO. 88159, respectively, or a homolog or a fragment thereof, is increased or generated, or if the activity Na+/K+ transporter is increased or generated in a non-human organism, like a microorganism or a plant cell, plant or part thereof, especially with mitochondrial localization, whereby the respective line discloses in column 7 the fine chemical leucine. For example, an increase of the leucine of at least 1 percent, particularly in a range of 59 to 144-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a 3-isopropylmalate dehydrogenase, or if the activity of the polypeptide Slr1517, preferably represented by SEQ ID NO. 107146, or a homolog or fragment thereof, or if the activity of a polypeptide encoded by a nucleic acid molecule comprising the nucleic acid SEQ ID NO. 107145, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 107145 or polypeptide SEQ ID NO. 107146, respectively, or a homolog or a fragment thereof, is increased or generated, or if the activity 3-isopropylmalate dehydrogenase is increased or generated in a non-human organism, like a microorganism or a plant cell, plant or part thereof, especially with mitochondrial localization, whereby the respective line discloses in column 7 the fine chemical valine. For example, an increase of the valine of at least 1 percent, particularly in a range of 27 to 33-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a photosystem II protein, or if the activity of the polypeptide Slr1739, preferably represented by SEQ ID NO. 58732, or a homolog or fragment thereof, or if the activity of a polypeptide encoded by a nucleic acid molecule comprising the nucleic acid SEQ ID NO. 58731, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 58731 or polypeptide SEQ ID NO. 58732, respectively, or a homolog or a fragment thereof, is increased or generated, or if the activity photosystem II protein is increased or generated in a non-human organism, like a microorganism or a plant cell, plant or part thereof, especially with plastidic localization, whereby the respective line discloses in column 7 the fine chemical isoleucine. For example, an increase of the isoleucine of at least 1 percent, particularly in a range of 54 to 308-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a photosystem II protein, or if the activity of the polypeptide Slr1739, preferably represented by SEQ ID NO. 58732, or a homolog or fragment thereof, or if the activity of a polypeptide encoded by a nucleic acid molecule comprising the nucleic acid SEQ ID NO. 58731, preferably the coding region thereof, or a homolog or fragment thereof, e.g. derived from Synechocystis sp., is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 58731 or polypeptide SEQ ID NO. 58732, respectively, or a homolog or a fragment thereof, is increased or generated, or if the activity photosystem II protein is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 148 to 331-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a photosystem II protein, or if the activity of the polypeptide Slr1739, preferably represented by SEQ ID NO. 58732, or a homolog or fragment thereof, or if the activity of a polypeptide encoded by a nucleic acid molecule comprising the nucleic acid SEQ ID NO. 58731, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 58731 or polypeptide SEQ ID NO. 58732, respectively, or a homolog or a fragment thereof, is increased or generated, or if the activity photosystem II protein is increased or generated in a non-human organism, like a microorganism or a plant cell, plant or part thereof, especially with plastidic localization, whereby the respective line discloses in column 7 the fine chemical valine. For example, an increase of the valine of at least 1 percent, particularly in a range of 37 to 107-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a phosphoadenosine phosphosulfate reductase , or if the activity of the polypeptide Slr1791, preferably represented by SEQ ID NO. 12141, or a homolog or fragment thereof, or if the activity of a polypeptide encoded by a nucleic acid molecule comprising the nucleic acid SEQ ID NO. 12140, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 12140 or polypeptide SEQ ID NO. 12141, respectively, or a homolog or a fragment thereof, is increased or generated, or if the activity phosphoadenosine phosphosulfate reductase is increased or generated in a non-human organism, like a microorganism or a plant cell, plant or part thereof, especially with plastidic localization, whereby the respective line discloses in column 7 the fine chemical valine. For example, an increase of the valine of at least 1 percent, particularly in a range of 25 to 33-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a riboflavin biosynthesis protein, or if the activity of the polypeptide Slr1882, preferably represented by SEQ ID NO. 59042, or a homolog or fragment thereof, or if the activity of a polypeptide encoded by a nucleic acid molecule comprising the nucleic acid SEQ ID NO. 59041, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 59041 or polypeptide SEQ ID NO. 59042, respectively, or a homolog or a fragment thereof, is increased or generated, or if the activity riboflavin biosynthesis protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 20 to 27-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a threonine dehydratase, or if the activity of the polypeptide Slr2072, preferably represented by SEQ ID NO. 12342, or a homolog or fragment thereof, or if the activity of a polypeptide encoded by a nucleic acid molecule comprising the nucleic acid SEQ ID NO. 12341, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 12341 or polypeptide SEQ ID NO. 12342, respectively, or a homolog or a fragment thereof, is increased or generated, or if the activity threonine dehydratase is increased or generated in a non-human organism, like a microorganism or a plant cell, plant or part thereof, especially with mitochondrial localization, whereby the respective line discloses in column 7 the fine chemical isoleucine. For example, an increase of the isoleucine of at least 1 percent, particularly in a range of 592 to 9865-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a threonine dehydratase, or if the activity of the polypeptide Slr2072, preferably represented by SEQ ID NO. 12342, or a homolog or fragment thereof, or if the activity of a polypeptide encoded by a nucleic acid molecule comprising the nucleic acid SEQ ID NO. 12341, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 12341 or polypeptide SEQ ID NO. 12342, respectively, or a homolog or a fragment thereof, is increased or generated, or if the activity threonine dehydratase is increased or generated in a non-human organism, like a microorganism or a plant cell, plant or part thereof, especially with plastidic localization, whereby the respective line discloses in column 7 the fine chemical isoleucine. For example, an increase of the isoleucine of at least 1 percent, particularly in a range of 1626 to 10965-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a threonine dehydratase, or if the activity of the polypeptide Slr2072, preferably represented by SEQ ID NO. 12342, or a homolog or fragment thereof, or if the activity of a polypeptide encoded by a nucleic acid molecule comprising the nucleic acid SEQ ID NO. 12341, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 12341 or polypeptide SEQ ID NO. 12342, respectively, or a homolog or a fragment thereof, is increased or generated, or if the activity threonine dehydratase is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 50 to 250-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a threonine dehydratase, or if the activity of the polypeptide Slr2072, preferably represented by SEQ ID NO. 12342, or a homolog or fragment thereof, or if the activity of a polypeptide encoded by a nucleic acid molecule comprising the nucleic acid SEQ ID NO. 12341, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 12341 or polypeptide SEQ ID NO. 12342, respectively, or a homolog or a fragment thereof, is increased or generated, or if the activity threonine dehydratase is increased or generated in a non-human organism, like a microorganism or a plant cell, plant or part thereof, especially with mitochondrial localization, whereby the respective line discloses in column 7 the fine chemical leucine. For example, an increase of the leucine of at least 1 percent, particularly in a range of 60 to 214-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a threonine dehydratase, or if the activity of the polypeptide Slr2072, preferably represented by SEQ ID NO. 12342, or a homolog or fragment thereof, or if the activity of a polypeptide encoded by a nucleic acid molecule comprising the nucleic acid SEQ ID NO. 12341, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 12341 or polypeptide SEQ ID NO. 12342, respectively, or a homolog or a fragment thereof, is increased or generated, or if the activity threonine dehydratase is increased or generated in a non-human organism, like a microorganism or a plant cell, plant or part thereof, especially with mitochondrial localization, whereby the respective line discloses in column 7 the fine chemical valine. For example, an increase of the valine of at least 1 percent, particularly in a range of 23 to 54-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a threonine dehydratase, or if the activity of the polypeptide Slr2072, preferably represented by SEQ ID NO. 12342, or a homolog or fragment thereof, or if the activity of a polypeptide encoded by a nucleic acid molecule comprising the nucleic acid SEQ ID NO. 12341, preferably the coding region thereof, or a homolog or fragment thereof, e.g. derived from Synechocystis sp., is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 12341 or polypeptide SEQ ID NO. 12342, respectively, or a homolog or a fragment thereof, is increased or generated, or if the activity threonine dehydratase is increased or generated in a non-human organism, like a microorganism or a plant cell, plant or part thereof, especially with plastidic localization, whereby the respective line discloses in column 7 the fine chemical valine. For example, an increase of the valine of at least 1 percent, particularly in a range of 24 to 64-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a short-chain alcohol dehydrogenase family, or if the activity of the polypeptide Slr2124, preferably represented by SEQ ID NO. 59371, or a homolog or fragment thereof, or if the activity of a polypeptide encoded by a nucleic acid molecule comprising the nucleic acid SEQ ID NO. 59370, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 59370 or polypeptide SEQ ID NO. 59371, respectively, or a homolog or a fragment thereof, is increased or generated, or if the activity short-chain alcohol dehydrogenase family is increased or generated in a non-human organism, like a microorganism or a plant cell, plant or part thereof, especially with plastidic localization, whereby the respective line discloses in column 7 the fine chemical valine. For example, an increase of the valine of at least 1 percent, particularly in a range of 34 to 73-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a Sec-independent protein translocase subunit, or if the activity of the polypeptide TTC0019, preferably represented by SEQ ID NO. 12699, or a homolog or fragment thereof, or if the activity of a polypeptide encoded by a nucleic acid molecule comprising the nucleic acid SEQ ID NO. 12698, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 12698 or polypeptide SEQ ID NO. 12699, respectively, or a homolog or a fragment thereof, is increased or generated, or if the activity Sec-independent protein translocase subunit is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 43 to 847-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a Sec-independent protein translocase subunit, or if the activity of the polypeptide TTC0019, preferably represented by SEQ ID NO. 12699, or a homolog or fragment thereof, or if the activity of a polypeptide encoded by a nucleic acid molecule comprising the nucleic acid SEQ ID NO. 12698, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 12698 or polypeptide SEQ ID NO. 12699, respectively, or a homolog or a fragment thereof, is increased or generated, or if the activity Sec-independent protein translocase subunit is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 60 to 889-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a Sec-independent protein translocase subunit, or if the activity of the polypeptide TTC0019, preferably represented by SEQ ID NO. 12699, or a homolog or fragment thereof, or if the activity of a polypeptide encoded by a nucleic acid molecule comprising the nucleic acid SEQ ID NO. 12698, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 12698 or polypeptide SEQ ID NO. 12699, respectively, or a homolog or a fragment thereof, is increased or generated, or if the activity Sec-independent protein translocase subunit is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 30 to 402-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a 2-isopropylmalate synthase, or if the activity of the polypeptide TTC0849, preferably represented by SEQ ID NO. 107557, or a homolog or fragment thereof, or if the activity of a polypeptide encoded by a nucleic acid molecule comprising the nucleic acid SEQ ID NO. 107556, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 107556 or polypeptide SEQ ID NO. 107557, respectively, or a homolog or a fragment thereof, is increased or generated, or if the activity 2-isopropylmalate synthase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 54 to 92-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a 2-isopropylmalate synthase, or if the activity of the polypeptide TTC0849, preferably represented by SEQ ID NO. 107557, or a homolog or fragment thereof, or if the activity of a polypeptide encoded by a nucleic acid molecule comprising the nucleic acid SEQ ID NO. 107556, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 107556 or polypeptide SEQ ID NO. 107557, respectively, or a homolog or a fragment thereof, is increased or generated, or if the activity 2-isopropylmalate synthase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 51 to 112-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a TTC1386-protein, or if the activity of the polypeptide TTC1386, preferably represented by SEQ ID NO. 62080, or a homolog or fragment thereof, or if the activity of a polypeptide encoded by a nucleic acid molecule comprising the nucleic acid SEQ ID NO. 62079, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 62079 or polypeptide SEQ ID NO. 62080, respectively, or a homolog or a fragment thereof, is increased or generated, or if the activity TTC1386-protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 56 to 744-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a TTC1386-protein, or if the activity of the polypeptide TTC1386, preferably represented by SEQ ID NO. 62080, or a homolog or fragment thereof, or if the activity of a polypeptide encoded by a nucleic acid molecule comprising the nucleic acid SEQ ID NO. 62079, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 62079 or polypeptide SEQ ID NO. 62080, respectively, or a homolog or a fragment thereof, is increased or generated, or if the activity TTC1386-protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 76 to 644-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a TTC1386-protein, or if the activity of the polypeptide TTC1386, preferably represented by SEQ ID NO. 62080, or a homolog or fragment thereof, or if the activity of a polypeptide encoded by a nucleic acid molecule comprising the nucleic acid SEQ ID NO. 62079, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 62079 or polypeptide SEQ ID NO. 62080, respectively, or a homolog or a fragment thereof, is increased or generated, or if the activity TTC1386-protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 38 to 334-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a homocitrate synthase, or if the activity of the polypeptide TTC1550, preferably represented by SEQ ID NO. 12975, or a homolog or fragment thereof, or if the activity of a polypeptide encoded by a nucleic acid molecule comprising the nucleic acid SEQ ID NO. 12974, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 12974 or polypeptide SEQ ID NO. 12975, respectively, or a homolog or a fragment thereof, is increased or generated, or if the activity homocitrate synthase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 70 to 355-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a homocitrate synthase, or if the activity of the polypeptide TTC1550, preferably represented by SEQ ID NO. 12975, or a homolog or fragment thereof, or if the activity of a polypeptide encoded by a nucleic acid molecule comprising the nucleic acid SEQ ID NO. 12974, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 12974 or polypeptide SEQ ID NO. 12975, respectively, or a homolog or a fragment thereof, is increased or generated, or if the activity homocitrate synthase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 66 to 340-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a homocitrate synthase, or if the activity of the polypeptide TTC1550, preferably represented by SEQ ID NO. 12975, or a homolog or fragment thereof, or if the activity of a polypeptide encoded by a nucleic acid molecule comprising the nucleic acid SEQ ID NO. 12974, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 12974 or polypeptide SEQ ID NO. 12975, respectively, or a homolog or a fragment thereof, is increased or generated, or if the activity homocitrate synthase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 29 to 129-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a YAL013W-protein, or if the activity of the polypeptide Yal013w, preferably represented by SEQ ID NO. 108093, or a homolog or fragment thereof, or if the activity of a polypeptide encoded by a nucleic acid molecule comprising the nucleic acid SEQ ID NO. 108092, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 108092 or polypeptide SEQ ID NO. 108093, respectively, or a homolog or a fragment thereof, is increased or generated, or if the activity YAL013W-protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 44 to 144-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a cell division control protein, or if the activity of the polypeptide Ybr160w, preferably represented by SEQ ID NO. 62718, or a homolog or fragment thereof, or if the activity of a polypeptide encoded by a nucleic acid molecule comprising the nucleic acid SEQ ID NO. 62717, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 62717 or polypeptide SEQ ID NO. 62718, respectively, or a homolog or a fragment thereof, is increased or generated, or if the activity cell division control protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 20 to 55-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a phosphoglycerate kinase, or if the activity of the polypeptide Ycr012w, preferably represented by SEQ ID NO. 108114, or a homolog or fragment thereof, or if the activity of a polypeptide encoded by a nucleic acid molecule comprising the nucleic acid SEQ ID NO. 108113, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 108113 or polypeptide SEQ ID NO. 108114, respectively, or a homolog or a fragment thereof, is increased or generated, or if the activity phosphoglycerate kinase is increased or generated in a non-human organism, like a microorganism or a plant cell, plant or part thereof, especially with plastidic localization, whereby the respective line discloses in column 7 the fine chemical isoleucine. For example, an increase of the isoleucine of at least 1 percent, particularly in a range of 29 to 218 -percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a phosphoglycerate kinase, or if the activity of the polypeptide Ycr012w, preferably represented by SEQ ID NO. 108114, or a homolog or fragment thereof, or if the activity of a polypeptide encoded by a nucleic acid molecule comprising the nucleic acid SEQ ID NO. 108113, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 108113 or polypeptide SEQ ID NO. 108114, respectively, or a homolog or a fragment thereof, is increased or generated, or if the activity phosphoglycerate kinase is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 33 to 136-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a branched-chain amino acid permease, or if the activity of the polypeptide Ydr046c, preferably represented by SEQ ID NO. 63545, or a homolog or fragment thereof, or if the activity of a polypeptide encoded by a nucleic acid molecule comprising the nucleic acid SEQ ID NO. 63544, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 63544 or polypeptide SEQ ID NO. 63545, respectively, or a homolog or a fragment thereof, is increased or generated, or if the activity branched-chain amino acid permease is increased or generated in a non-human organism, like a microorganism or a plant cell, plant or part thereof, especially with plastidic localization, whereby the respective line discloses in column 7 the fine chemical isoleucine. For example, an increase of the isoleucine of at least 1 percent, particularly in a range of 50 to 70-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a branched-chain amino acid permease, or if the activity of the polypeptide Ydr046c, preferably represented by SEQ ID NO. 63545, or a homolog or fragment thereof, or if the activity of a polypeptide encoded by a nucleic acid molecule comprising the nucleic acid SEQ ID NO. 63544, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 63544 or polypeptide SEQ ID NO. 63545, respectively, or a homolog or a fragment thereof, is increased or generated, or if the activity branched-chain amino acid permease is increased or generated in a non-human organism, like a microorganism or a plant cell, plant or part thereof, especially with plastidic localization, whereby the respective line discloses in column 7 the fine chemical valine. For example, an increase of the valine of at least 1 percent, particularly in a range of 21 to 30-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a ydr183w-protein, or if the activity of the polypeptide Ydr183w, preferably represented by SEQ ID NO. 90104, or a homolog or fragment thereof, or if the activity of a polypeptide encoded by a nucleic acid molecule comprising the nucleic acid SEQ ID NO. 90103, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 90103 or polypeptide SEQ ID NO. 90104, respectively, or a homolog or a fragment thereof, is increased or generated, or if the activity ydr183w-protein is increased or generated in a non-human organism, like a microorganism or a plant cell, plant or part thereof, especially with plastidic localization, whereby the respective line discloses in column 7 the fine chemical valine. For example, an increase of the valine of at least 1 percent, particularly in a range of 30 to 48-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a hexose transporter, or if the activity of the polypeptide Yel069c, preferably represented by SEQ ID NO. 97299, or a homolog or fragment thereof, or if the activity of a polypeptide encoded by a nucleic acid molecule comprising the nucleic acid SEQ ID NO. 97298, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 97298 or polypeptide SEQ ID NO. 97299, respectively, or a homolog or a fragment thereof, is increased or generated, or if the activity hexose transporter is increased or generated in a non-human organism, like a microorganism or a plant cell, plant or part thereof, especially with plastidic localization, whereby the respective line discloses in column 7 the fine chemical isoleucine. For example, an increase of the isoleucine of at least 1 percent, particularly in a range of 38 to 58-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a ygl237c-protein, or if the activity of the polypeptide Ygl237c, preferably represented by SEQ ID NO. 64178, or a homolog or fragment thereof, or if the activity of a polypeptide encoded by a nucleic acid molecule comprising the nucleic acid SEQ ID NO. 64177, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 64177 or polypeptide SEQ ID NO. 64178, respectively, or a homolog or a fragment thereof, is increased or generated, or if the activity ygl237c-protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 39 to 294-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a ygl237c-protein, or if the activity of the polypeptide Ygl237c, preferably represented by SEQ ID NO. 64178, or a homolog or fragment thereof, or if the activity of a polypeptide encoded by a nucleic acid molecule comprising the nucleic acid SEQ ID NO. 64177, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 64177 or polypeptide SEQ ID NO. 64178, respectively, or a homolog or a fragment thereof, is increased or generated, or if the activity ygl237c-protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 80 to 355-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a YGR193C-protein, or if the activity of the polypeptide Ygr193c, preferably represented by SEQ ID NO. 108488, or a homolog or fragment thereof, or if the activity of a polypeptide encoded by a nucleic acid molecule comprising the nucleic acid SEQ ID NO. 108487, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 108487 or polypeptide SEQ ID NO. 108488, respectively, or a homolog or a fragment thereof, is increased or generated, or if the activity YGR193C-protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 54 to 294-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a yhr006w-protein, or if the activity of the polypeptide Yhr006w, preferably represented by SEQ ID NO. 90285, or a homolog or fragment thereof, or if the activity of a polypeptide encoded by a nucleic acid molecule comprising the nucleic acid SEQ ID NO. 90284, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 90284 or polypeptide SEQ ID NO. 90285, respectively, or a homolog or a fragment thereof, is increased or generated, or if the activity yhr006w-protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 54 to 114-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a yhr006w-protein, or if the activity of the polypeptide Yhr006w, preferably represented by SEQ ID NO. 90285, or a homolog or fragment thereof, or if the activity of a polypeptide encoded by a nucleic acid molecule comprising the nucleic acid SEQ ID NO. 90284, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 90284 or polypeptide SEQ ID NO. 90285, respectively, or a homolog or a fragment thereof, is increased or generated, or if the activity yhr006w-protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 71 to 161-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a H/ACA ribonucleoprotein complex subunit 3, or if the activity of the polypeptide Yhr072w-a, preferably represented by SEQ ID NO. 64471, or a homolog or fragment thereof, or if the activity of a polypeptide encoded by a nucleic acid molecule comprising the nucleic acid SEQ ID NO. 64470, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 64470 or polypeptide SEQ ID NO. 64471, respectively, or a homolog or a fragment thereof, is increased or generated, or if the activity H/ACA ribonucleoprotein complex subunit 3 is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 24 to 181-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a H/ACA ribonucleoprotein complex subunit 3, or if the activity of the polypeptide Yhr072w-a, preferably represented by SEQ ID NO. 64471, or a homolog or fragment thereof, or if the activity of a polypeptide encoded by a nucleic acid molecule comprising the nucleic acid SEQ ID NO. 64470, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 64470 or polypeptide SEQ ID NO. 64471, respectively, or a homolog or a fragment thereof, is increased or generated, or if the activity H/ACA ribonucleoprotein complex subunit 3 is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 40 to 188-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a glycerol-3-phosphate dehydrogenase, or if the activity of the polypeptide Yi1155c, preferably represented by SEQ ID NO. 108515, or a homolog or fragment thereof, or if the activity of a polypeptide encoded by a nucleic acid molecule comprising the nucleic acid SEQ ID NO. 108514, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 108514 or polypeptide SEQ ID NO. 108515, respectively, or a homolog or a fragment thereof, is increased or generated, or if the activity glycerol-3-phosphate dehydrogenase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 22 to 71-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a glycerol-3-phosphate dehydrogenase, or if the activity of the polypeptide Yil155c, preferably represented by SEQ ID NO. 108515, or a homolog or fragment thereof, or if the activity of a polypeptide encoded by a nucleic acid molecule comprising the nucleic acid SEQ ID NO. 108514, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 108514 or polypeptide SEQ ID NO. 108515, respectively, or a homolog or a fragment thereof, is increased or generated, or if the activity glycerol-3-phosphate dehydrogenase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 41 to 106-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a homoserine dehydrogenase, or if the activity of the polypeptide Yjr139c, preferably represented by SEQ ID NO. 14886, or a homolog or fragment thereof, or if the activity of a polypeptide encoded by a nucleic acid molecule comprising the nucleic acid SEQ ID NO. 14885, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 14885 or polypeptide SEQ ID NO. 14886, respectively, or a homolog or a fragment thereof, is increased or generated, or if the activity homoserine dehydrogenase is increased or generated in a non-human organism, like a microorganism or a plant cell, plant or part thereof, especially with plastidic localization, whereby the respective line discloses in column 7 the fine chemical isoleucine. For example, an increase of the isoleucine of at least 1 percent, particularly in a range of 59 to 86-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a branched chain aminotransferase, or if the activity of the polypeptide Yjr148w, preferably represented by SEQ ID NO. 90410, or a homolog or fragment thereof, or if the activity of a polypeptide encoded by a nucleic acid molecule comprising the nucleic acid SEQ ID NO. 90409, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 90409 or polypeptide SEQ ID NO. 90410, respectively, or a homolog or a fragment thereof, is increased or generated, or if the activity branched chain aminotransferase is increased or generated in a non-human organism, like a microorganism or a plant cell, plant or part thereof, especially with plastidic localization, whereby the respective line discloses in column 7 the fine chemical isoleucine. For example, an increase of the isoleucine of at least 1 percent, particularly in a range of 109 to 273-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a branched chain aminotransferase, or if the activity of the polypeptide Yjr148w, preferably represented by SEQ ID NO. 90410, or a homolog or fragment thereof, or if the activity of a polypeptide encoded by a nucleic acid molecule comprising the nucleic acid SEQ ID NO. 90409, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 90409 or polypeptide SEQ ID NO. 90410, respectively, or a homolog or a fragment thereof, is increased or generated, or if the activity branched chain aminotransferase is increased or generated in a non-human organism, like a microorganism or a plant cell, plant or part thereof, especially with plastidic localization, whereby the respective line discloses in column 7 the fine chemical valine. For example, an increase of the valine of at least 1 percent, particularly in a range of 74 to 170-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a polygalacturonase, or if the activity of the polypeptide Yjr153w, preferably represented by SEQ ID NO. 66275, or a homolog or fragment thereof, or if the activity of a polypeptide encoded by a nucleic acid molecule comprising the nucleic acid SEQ ID NO. 66274, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 66274 or polypeptide SEQ ID NO. 66275, respectively, or a homolog or a fragment thereof, is increased or generated, or if the activity polygalacturonase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 66 to 273-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a polygalacturonase, or if the activity of the polypeptide Yjr153w, preferably represented by SEQ ID NO. 66275, or a homolog or fragment thereof, or if the activity of a polypeptide encoded by a nucleic acid molecule comprising the nucleic acid SEQ ID NO. 66274, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 66274 or polypeptide SEQ ID NO. 66275, respectively, or a homolog or a fragment thereof, is increased or generated, or if the activity polygalacturonase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 64 to 364-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a polygalacturonase, or if the activity of the polypeptide Yjr153w, preferably represented by SEQ ID NO. 66275, or a homolog or fragment thereof, or if the activity of a polypeptide encoded by a nucleic acid molecule comprising the nucleic acid SEQ ID NO. 66274, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 66274 or polypeptide SEQ ID NO. 66275, respectively, or a homolog or a fragment thereof, is increased or generated, or if the activity polygalacturonase is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 28 to 110-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a YKL038W-protein, or if the activity of the polypeptide YKL038W, preferably represented by SEQ ID NO. 90603, or a homolog or fragment thereof, or if the activity of a polypeptide encoded by a nucleic acid molecule comprising the nucleic acid SEQ ID NO. 90602, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 90602 or polypeptide SEQ ID NO. 90603, respectively, or a homolog or a fragment thereof, is increased or generated, or if the activity YKL038W-protein is increased or generated in a non-human organism, like a microorganism or a plant cell, plant or part thereof, especially with plastidic localization, whereby the respective line discloses in column 7 the fine chemical isoleucine. For example, an increase of the isoleucine of at least 1 percent, particularly in a range of 51 to 53-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a YKL038W-protein, or if the activity of the polypeptide YKL038W, preferably represented by SEQ ID NO. 90603, or a homolog or fragment thereof, or if the activity of a polypeptide encoded by a nucleic acid molecule comprising the nucleic acid SEQ ID NO. 90602, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 90602 or polypeptide SEQ ID NO. 90603, respectively, or a homolog or a fragment thereof, is increased or generated, or if the activity YKL038W-protein is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 50 to 59-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a YLR224W-protein, or if the activity of the polypeptide Ylr224w, preferably represented by SEQ ID NO. 108724, or a homolog or fragment thereof, or if the activity of a polypeptide encoded by a nucleic acid molecule comprising the nucleic acid SEQ ID NO. 108723, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 108723 or polypeptide SEQ ID NO. 108724, respectively, or a homolog or a fragment thereof, is increased or generated, or if the activity YLR224W-protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 25 to 50-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a mitochondrial dicarboxylate transporter, or if the activity of the polypeptide Ylr348c, preferably represented by SEQ ID NO. 98244, or a homolog or fragment thereof, or if the activity of a polypeptide encoded by a nucleic acid molecule comprising the nucleic acid SEQ ID NO. 98243, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 98243 or polypeptide SEQ ID NO. 98244, respectively, or a homolog or a fragment thereof, is increased or generated, or if the activity mitochondrial dicarboxylate transporter is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 55 to 83-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a adenylosuccinate lyase, or if the activity of the polypeptide Ylr359w, preferably represented by SEQ ID NO. 67191, or a homolog or fragment thereof, or if the activity of a polypeptide encoded by a nucleic acid molecule comprising the nucleic acid SEQ ID NO. 67190, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 67190 or polypeptide SEQ ID NO. 67191, respectively, or a homolog or a fragment thereof, is increased or generated, or if the activity adenylosuccinate lyase is increased or generated in a non-human organism, like a microorganism or a plant cell, plant or part thereof, especially with plastidic localization, whereby the respective line discloses in column 7 the fine chemical isoleucine. For example, an increase of the isoleucine of at least 1 percent, particularly in a range of 59 to 451-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a adenylosuccinate lyase, or if the activity of the polypeptide Ylr359w, preferably represented by SEQ ID NO. 67191, or a homolog or fragment thereof, or if the activity of a polypeptide encoded by a nucleic acid molecule comprising the nucleic acid SEQ ID NO. 67190, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 67190 or polypeptide SEQ ID NO. 67191, respectively, or a homolog or a fragment thereof, is increased or generated, or if the activity adenylosuccinate lyase is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 83 to 685-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a adenylosuccinate lyase, or if the activity of the polypeptide Ylr359w, preferably represented by SEQ ID NO. 67191, or a homolog or fragment thereof, or if the activity of a polypeptide encoded by a nucleic acid molecule comprising the nucleic acid SEQ ID NO. 67190, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 67190 or polypeptide SEQ ID NO. 67191, respectively, or a homolog or a fragment thereof, is increased or generated, or if the activity adenylosuccinate lyase is increased or generated in a non-human organism, like a microorganism or a plant cell, plant or part thereof, especially with plastidic localization, whereby the respective line discloses in column 7 the fine chemical valine. For example, an increase of the valine of at least 1 percent, particularly in a range of 20 to 168-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a phosphate transporter, or if the activity of the polypeptide Yml123c, preferably represented by SEQ ID NO. 91786, or a homolog or fragment thereof, or if the activity of a polypeptide encoded by a nucleic acid molecule comprising the nucleic acid SEQ ID NO. 91785, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 91785 or polypeptide SEQ ID NO. 91786, respectively, or a homolog or a fragment thereof, is increased or generated, or if the activity phosphate transporter is increased or generated in a non-human organism, like a microorganism or a plant cell, plant or part thereof, especially with plastidic localization, whereby the respective line discloses in column 7 the fine chemical leucine. For example, an increase of the leucine of at least 1 percent, particularly in a range of 65 to 96-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a acetyl-CoA acetyltransferase, or if the activity of the polypeptide Ypl028w, preferably represented by SEQ ID NO. 92669, or a homolog or fragment thereof, or if the activity of a polypeptide encoded by a nucleic acid molecule comprising the nucleic acid SEQ ID NO. 92668, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 92668 or polypeptide SEQ ID NO. 92669, respectively, or a homolog or a fragment thereof, is increased or generated, or if the activity acetyl-CoA acetyltransferase is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 29 to 182-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a acetyl-CoA acetyltransferase, or if the activity of the polypeptide Ypl028w, preferably represented by SEQ ID NO. 92669, or a homolog or fragment thereof, or if the activity of a polypeptide encoded by a nucleic acid molecule comprising the nucleic acid SEQ ID NO. 92668, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 92668 or polypeptide SEQ ID NO. 92669, respectively, or a homolog or a fragment thereof, is increased or generated, or if the activity acetyl-CoA acetyltransferase is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 32 to 142-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a cyclin, or if the activity of the polypeptide Ypr119w, preferably represented by SEQ ID NO. 98446, or a homolog or fragment thereof, or if the activity of a polypeptide encoded by a nucleic acid molecule comprising the nucleic acid SEQ ID NO. 98445, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 98445 or polypeptide SEQ ID NO. 98446, respectively, or a homolog or a fragment thereof, is increased or generated, or if the activity cyclin is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 33 to 57-percent is conferred as compared to a corresponding non-transformed wild type non-human organism.

Accordingly, in one embodiment, an increase of isoleucine in a non-human organism, as compared to a corresponding non-transformed wild type non-human organism, is conferred in the process of the invention, if the activity of a polypeptide showing the activity of a Zm_{—}4842_BE510522-protein, or if the activity of the polypeptide Zm_{—}4842_BE510522, preferably represented by SEQ ID NO. 68414, or a homolog or fragment thereof, or if the activity of a polypeptide encoded by a nucleic acid molecule comprising the nucleic acid SEQ ID NO. 68413, preferably the coding region thereof, or a homolog or fragment thereof, e.g. derived from Zea mays, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 68413 or polypeptide SEQ ID NO. 68414, respectively, or a homolog or a fragment thereof, is increased or generated, or if the activity Zm_{—}4842_BE510522-protein is increased or generated in a non-human organism, like a microorganism or a plant cell, plant or part thereof, especially with non-targeted localization, whereby the respective line discloses in column 7 the fine chemical isoleucine. For example, an increase of the isoleucine of at least 1 percent, particularly in a range of 42 to 313-percent is conferred as compared to a corresponding non-transformed wild type non-human organism.

Accordingly, in one embodiment, an increase of leucine in a non-human organism, as compared to a corresponding non-transformed wild type non-human organism, is conferred in the process of the invention, if the activity of a polypeptide showing the activity of a Zm_{—}4842_BE510522-protein, or if the activity of the polypeptide Zm_{—}4842_BE510522, preferably represented by SEQ ID NO. 68414, or a homolog or fragment thereof, or if the activity of a polypeptide encoded by a nucleic acid molecule comprising the nucleic acid SEQ ID NO. 68413, preferably the coding region thereof, or a homolog or fragment thereof, e.g. derived from Zea mays, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 68413 or polypeptide SEQ ID NO. 68414, respectively, or a homolog or a fragment thereof, is increased or generated, or if the activity Zm_{—}4842_BE510522-protein is increased or generated in a non-human organism, like a microorganism or a plant cell, plant or part thereof, especially with non-targeted localization, whereby the respective line discloses in column 7 the fine chemical leucine. For example, an increase of the leucine of at least 1 percent, particularly in a range of 53 to 336-percent is conferred as compared to a corresponding non-transformed wild type non-human organism.

Accordingly, in one embodiment, an increase of valine in a non-human organism, as compared to a corresponding non-transformed wild type non-human organism, is conferred in the process of the invention, if the activity of a polypeptide showing the activity of a Zm_{—}4842_BE510522-protein, or if the activity of the polypeptide Zm_{—}4842_BE510522, preferably represented by SEQ ID NO. 68414, or a homolog or fragment thereof, or if the activity of a polypeptide encoded by a nucleic acid molecule comprising the nucleic acid SEQ ID NO. 68413, preferably the coding region thereof, or a homolog or fragment thereof, e.g. derived from Zea mays, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 6, column 5 or 8 in the respective same line as the nucleic acid molecule SEQ ID NO. 68413 or polypeptide SEQ ID NO. 68414, respectively, or a homolog or a fragment thereof, is increased or generated, or if the activity Zm_{—}4842_BE510522-protein is increased or generated in a non-human organism, like a microorganism or a plant cell, plant or part thereof, especially with non-targeted localization, whereby the respective line discloses in column 7 the fine chemical valine. For example, an increase of the valine of at least 1 percent, particularly in a range of 29 to 116-percent is conferred as compared to a corresponding non-transformed wild type non-human organism.

[0096.1.1.6] to [0103.1.1.6] for the disclosure of these paragraphs see [0096.1.1.1] to [0103.1.1.1] above.

In this paragraph “A” means “Goffeau et al., Science 274 (5287), 546 (1996)”, “B” means “Blattner et al., Science 277 (5331), 1453 (1997)”, “C” means “Nakamura et al., Nucleic Acids Res. 28(1), 72 (2000)”, “D” means “deposition of the sequences to the EMBL/GenBank/DDBJ databases under “Sequencing of the draft genome assembly of Azotobacter vinelandii AvOP”. in June 2005 by Copeland et al.”, “E” means “Henne et al., Nat. Biotechnol. 22 (5), 547 (2004)”, “F” means “Kaul et al., Nature 408 (6814), 796 (2000)”, and “G” means “Matsumoto et al., Nature 436 (7052), 793 (2005)”.

The nucleic acid sequence of 47266012_SOYBEAN from Glycine max, e.g. as shown in the respective line in column 5 of Table I, application no. 6, is unpublished. And the activity of the gene product thereof is the activity of 47266012-protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “47266012-protein”, especially from Glycine max or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said 47266012_SOYBEAN, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said 47266012_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said 47266012_SOYBEAN, or a functional equivalent or a homolog thereof as depicted in column 8 of Table II, application no. 6, preferably a homolog or functional equivalent as depicted in column 8 of Table II B, application no. 6, and being depicted in the same respective line as said 47266012_SOYBEAN, and preferably the activity is increased non-targeted, whereby the respective line discloses in column 7 the fine chemical isoleucine.

Accordingly, in one embodiment, the molecule which activity is to be increased in the process of the invention is the gene product with an activity as a “47266012-protein”, preferably it is the molecule of section (a) or (b) of this paragraph.

In particular, it was observed that in plants, especially in Arabidopsis thaliana, increasing or generating the activity of a gene product non-targeted with the activity of a “47266012-protein”, preferably being encoded by a gene comprising the nucleic acid sequence SEQ ID NO. 15187, preferably the coding region thereof, conferred the production of or the increase in isoleucine compared with the wild type control.

The nucleic acid sequence of 51340801_CANOLA from Brassica napus, e.g. as shown in the respective line in column 5 of Table I, application no. 6, is unpublished. And the activity of the gene product thereof is the activity of pyruvate kinase.

Accordingly, in one embodiment, the process of the present invention for producing leucine in a non-human organism, like a microorganism or a plant or a part thereof, comprises increasing or generating the activity of a gene product with the activity of a gene product conferring the activity of “pyruvate kinase”, especially from Brassica napus or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said 51340801_CANOLA, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said 51340801_CANOLA, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said 51340801_CANOLA, or a functional equivalent or a homolog thereof as depicted in column 8 of Table II, application no. 6, preferably a homolog or functional equivalent as depicted in column 8 of Table II B, application no. 6, and being depicted in the same respective line as said 51340801_CANOLA, and preferably the activity is increased plastidic, whereby the respective line discloses in column 7 the fine chemical leucine.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 15532, preferably the coding region thereof, conferred the production of or the increase in leucine compared with the wild type control.

The nucleic acid sequence of 51340801_CANOLA from Brassica napus, e.g. as shown in the respective line in column 5 of Table I, application no. 6, is unpublished. And the activity of the gene product thereof is the activity of pyruvate kinase.

Accordingly, in one embodiment, the process of the present invention for producing valine in a non-human organism, like a microorganism or a plant or a part thereof, comprises increasing or generating the activity of a gene product with the activity of a gene product conferring the activity of “pyruvate kinase”, especially from Brassica napus or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said 51340801_CANOLA, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said 51340801_CANOLA, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said 51340801_CANOLA, or a functional equivalent or a homolog thereof as depicted in column 8 of Table II, application no. 6, preferably a homolog or functional equivalent as depicted in column 8 of Table II B, application no. 6, and being depicted in the same respective line as said 51340801_CANOLA, and preferably the activity is increased plastidic, whereby the respective line discloses in column 7 the fine chemical valine.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 15532, preferably the coding region thereof, conferred the production of or the increase in valine compared with the wild type control.

The nucleic acid sequence of 59554615_SOYBEAN from Glycine max, e.g. as shown in the respective line in column 5 of Table I, application no. 6, is unpublished. And the activity of the gene product thereof is the activity of pyruvate kinase.

Accordingly, in one embodiment, the process of the present invention for producing valine in a non-human organism, like a microorganism or a plant or a part thereof, comprises increasing or generating the activity of a gene product with the activity of a gene product conferring the activity of “pyruvate kinase”, especially from Glycine max or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said 59554615_SOYBEAN, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said 59554615_SOYBEAN, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said 59554615_SOYBEAN, or a functional equivalent or a homolog thereof as depicted in column 8 of Table II, application no. 6, preferably a homolog or functional equivalent as depicted in column 8 of Table II B, application no. 6, and being depicted in the same respective line as said 59554615_SOYBEAN, and preferably the activity is increased plastidic, whereby the respective line discloses in column 7 the fine chemical valine.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 16263, preferably the coding region thereof, conferred the production of or the increase in valine compared with the wild type control.

The nucleic acid sequence of 59582753_SOYBEAN from Glycine max, e.g. as shown in the respective line in column 5 of Table I, application no. 6, is unpublished. And the activity of the gene product thereof is the activity of glucose-6-phosphate 1-dehydrogenase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “glucose-6-phosphate 1-dehydrogenase”, especially from Glycine max or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said 59582753_SOYBEAN, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said 59582753_SOYBEAN, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said 59582753_SOYBEAN, or a functional equivalent or a homolog thereof as depicted in column 8 of Table II, application no. 6, preferably a homolog or functional equivalent as depicted in column 8 of Table II B, application no. 6, and being depicted in the same respective line as said 59582753_SOYBEAN, and preferably the activity is increased plastidic, whereby the respective line discloses in column 7 the fine chemical isoleucine.

Accordingly, in one embodiment, the molecule which activity is to be increased in the process of the invention is the gene product with an activity as a “glucose-6-phosphate 1-dehydrogenase”, preferably it is the molecule of section (a) or (b) of this paragraph.

In particular, it was observed that in plants, especially in Arabidopsis thaliana, increasing or generating the activity of a gene product plastidic with the activity of a “glucose-6-phosphate 1-dehydrogenase”, preferably being encoded by a gene comprising the nucleic acid sequence SEQ ID NO. 16883, preferably the coding region thereof, conferred the production of or the increase in isoleucine compared with the wild type control.

The nucleic acid sequence of 59582753_SOYBEAN from Glycine max, e.g. as shown in the respective line in column 5 of Table I, application no. 6, is unpublished. And the activity of the gene product thereof is the activity of glucose-6-phosphate 1-dehydrogenase.

Accordingly, in one embodiment, the process of the present invention for producing leucine in a non-human organism, like a microorganism or a plant or a part thereof, comprises increasing or generating the activity of a gene product with the activity of a gene product conferring the activity of “glucose-6-phosphate 1-dehydrogenase”, especially from Glycine max or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said 59582753_SOYBEAN, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said 59582753_SOYBEAN, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said 59582753_SOYBEAN, or a functional equivalent or a homolog thereof as depicted in column 8 of Table II, application no. 6, preferably a homolog or functional equivalent as depicted in column 8 of Table II B, application no. 6, and being depicted in the same respective line as said 59582753_SOYBEAN, and preferably the activity is increased plastidic, whereby the respective line discloses in column 7 the fine chemical leucine.

Accordingly, in one embodiment, the molecule which activity is to be increased in the process of the invention is the gene product with an activity as a “glucose-6-phosphate 1-dehydrogenase”, preferably it is the molecule of section (a) or (b) of this paragraph.

In particular, it was observed that in plants, especially in Arabidopsis thaliana, increasing or generating the activity of a gene product plastidic with the activity of a “glucose-6-phosphate 1-dehydrogenase”, preferably being encoded by a gene comprising the nucleic acid sequence SEQ ID NO. 16883, preferably the coding region thereof, conferred the production of or the increase in leucine compared with the wild type control.

The nucleic acid sequence of 59582753_SOYBEAN from Glycine max, e.g. as shown in the respective line in column 5 of Table I, application no. 6, is unpublished. And the activity of the gene product thereof is the activity of glucose-6-phosphate 1-dehydrogenase.

Accordingly, in one embodiment, the process of the present invention for producing valine in a non-human organism, like a microorganism or a plant or a part thereof, comprises increasing or generating the activity of a gene product with the activity of a gene product conferring the activity of “glucose-6-phosphate 1-dehydrogenase”, especially from Glycine max or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said 59582753_SOYBEAN, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said 59582753_SOYBEAN, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said 59582753_SOYBEAN, or a functional equivalent or a homolog thereof as depicted in column 8 of Table II, application no. 6, preferably a homolog or functional equivalent as depicted in column 8 of Table II B, application no. 6, and being depicted in the same respective line as said 59582753_SOYBEAN, and preferably the activity is increased plastidic, whereby the respective line discloses in column 7 the fine chemical valine.

Accordingly, in one embodiment, the molecule which activity is to be increased in the process of the invention is the gene product with an activity as a “glucose-6-phosphate 1-dehydrogenase”, preferably it is the molecule of section (a) or (b) of this paragraph.

In particular, it was observed that in plants, especially in Arabidopsis thaliana, increasing or generating the activity of a gene product plastidic with the activity of a “glucose-6-phosphate 1-dehydrogenase”, preferably being encoded by a gene comprising the nucleic acid sequence SEQ ID NO. 16883, preferably the coding region thereof, conferred the production of or the increase in valine compared with the wild type control.

The nucleic acid sequence of At1g07430 from Arabidopsis thaliana, e.g. as shown in the respective line in column 5 of Table I, application no. 6, has been published in F. And the activity of the gene product thereof is the activity of protein phosphatase.

Accordingly, in one embodiment, the process of the present invention for producing valine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At1g07430, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At1g07430, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At1g07430, or a functional equivalent or a homolog thereof as depicted in column 8 of Table II, application no. 6, preferably a homolog or functional equivalent as depicted in column 8 of Table II B, application no. 6, and being depicted in the same respective line as said At1g07430, and preferably the activity is increased non-targeted, whereby the respective line discloses in column 7 the fine chemical valine.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 17451, preferably the coding region thereof, conferred the production of or the increase in valine compared with the wild type control.

The nucleic acid sequence of At1g14490 from Arabidopsis thaliana, e.g. as shown in the respective line in column 5 of Table I, application no. 6, has been published in F. And the activity of the gene product thereof is the activity of DNA-binding protein.

Accordingly, in one embodiment, the process of the present invention for producing valine in a non-human organism, like a microorganism or a plant or a part thereof, comprises increasing or generating the activity of a gene product with the activity of a gene product conferring the activity of “DNA-binding protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At1g14490, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At1g14490, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At1g14490, or a functional equivalent or a homolog thereof as depicted in column 8 of Table II, application no. 6, preferably a homolog or functional equivalent as depicted in column 8 of Table II B, application no. 6, and being depicted in the same respective line as said At1g14490, and preferably the activity is increased non-targeted, whereby the respective line discloses in column 7 the fine chemical valine.

Accordingly, in one embodiment, the molecule which activity is to be increased in the process of the invention is the gene product with an activity as a “DNA-binding protein”, preferably it is the molecule of section (a) or (b) of this paragraph.

In particular, it was observed that in plants, especially in Arabidopsis thaliana, increasing or generating the activity of a gene product non-targeted with the activity of a “DNA-binding protein”, preferably being encoded by a gene comprising the nucleic acid sequence SEQ ID NO. 385, preferably the coding region thereof, conferred the production of or the increase in valine compared with the wild type control.

The nucleic acid sequence of At1g19800 from Arabidopsis thaliana, e.g. as shown in the respective line in column 5 of Table I, application no. 6, has been published in F. And the activity of the gene product thereof is the activity of At1g19800-protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “At1g19800-protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said At1g19800, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At1g19800, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At1g19800, or a functional equivalent or a homolog thereof as depicted in column 8 of

Table II, application no. 6, preferably a homolog or functional equivalent as depicted in column 8 of Table II B, application no. 6, and being depicted in the same respective line as said At1g19800, and preferably the activity is increased non-targeted, whereby the respective line discloses in column 7 the fine chemical isoleucine.

Accordingly, in one embodiment, the molecule which activity is to be increased in the process of the invention is the gene product with an activity as a “At1g19800-protein”, preferably it is the molecule of section (a) or (b) of this paragraph.

In particular, it was observed that in plants, especially in Arabidopsis thaliana, increasing or generating the activity of a gene product non-targeted with the activity of a “At1g19800-protein”, preferably being encoded by a gene comprising the nucleic acid sequence SEQ ID NO. 17637, preferably the coding region thereof, conferred the production of or the increase in isoleucine compared with the wild type control.

The nucleic acid sequence of At1g19800 from Arabidopsis thaliana, e.g. as shown in the respective line in column 5 of Table I, application no. 6, has been published in F. And the activity of the gene product thereof is the activity of At1g19800-protein.

Accordingly, in one embodiment, the process of the present invention for producing leucine in a non-human organism, like a microorganism or a plant or a part thereof, comprises increasing or generating the activity of a gene product with the activity of a gene product conferring the activity of “At1g19800-protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said At1g19800, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At1g19800, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At1g19800, or a functional equivalent or a homolog thereof as depicted in column 8 of Table II, application no. 6, preferably a homolog or functional equivalent as depicted in column 8 of Table II B, application no. 6, and being depicted in the same respective line as said At1g19800, and preferably the activity is increased non-targeted, whereby the respective line discloses in column 7 the fine chemical leucine.

Accordingly, in one embodiment, the molecule which activity is to be increased in the process of the invention is the gene product with an activity as a “At1g19800-protein”, preferably it is the molecule of section (a) or (b) of this paragraph.

In particular, it was observed that in plants, especially in Arabidopsis thaliana, increasing or generating the activity of a gene product non-targeted with the activity of a “At1g19800-protein”, preferably being encoded by a gene comprising the nucleic acid sequence SEQ ID NO. 17637, preferably the coding region thereof, conferred the production of or the increase in leucine compared with the wild type control.

The nucleic acid sequence of At1g23110 from Arabidopsis thaliana, e.g. as shown in the respective line in column 5 of Table I, application no. 6, has been published in F. And the activity of the gene product thereof is the activity of At1g23110-protein.

Accordingly, in one embodiment, the process of the present invention for producing valine in a non-human organism, like a microorganism or a plant or a part thereof, comprises increasing or generating the activity of a gene product with the activity of a gene product conferring the activity of “At1g23110-protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At1g23110, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At1g23110, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At1g23110, or a functional equivalent or a homolog thereof as depicted in column 8 of Table II, application no. 6, preferably a homolog or functional equivalent as depicted in column 8 of Table II B, application no. 6, and being depicted in the same respective line as said At1g23110, and preferably the activity is increased non-targeted, whereby the respective line discloses in column 7 the fine chemical valine.

Accordingly, in one embodiment, the molecule which activity is to be increased in the process of the invention is the gene product with an activity as a “At1g23110-protein”, preferably it is the molecule of section (a) or (b) of this paragraph.

In particular, it was observed that in plants, especially in Arabidopsis thaliana, increasing or generating the activity of a gene product non-targeted with the activity of a “At1g23110-protein”, preferably being encoded by a gene comprising the nucleic acid sequence SEQ ID NO. 102086, preferably the coding region thereof, conferred the production of or the increase in valine compared with the wild type control.

The nucleic acid sequence of At1g26830 from Arabidopsis thaliana, e.g. as shown in the respective line in column 5 of Table I, application no. 6, has been published in F. And the activity of the gene product thereof is the activity of cullin.

Accordingly, in one embodiment, the process of the present invention for producing isoleucine in a non-human organism, like a microorganism or a plant or a part thereof, comprises increasing or generating the activity of a gene product with the activity of a gene product conferring the activity of “cullin”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said At1g26830, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At1g26830, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At1g26830,or a functional equivalent or a homolog thereof as depicted in column 8 of Table II, application no. 6, preferably a homolog or functional equivalent as depicted in column 8 of Table II B, application no. 6, and being depicted in the same respective line as said At1g26830, and preferably the activity is increased non-targeted, whereby the respective line discloses in column 7 the fine chemical isoleucine.

Accordingly, in one embodiment, the molecule which activity is to be increased in the process of the invention is the gene product with an activity as a “cullin”, preferably it is the molecule of section (a) or (b) of this paragraph.

In particular, it was observed that in plants, especially in Arabidopsis thaliana, increasing or generating the activity of a gene product non-targeted with the activity of a “cullin”, preferably being encoded by a gene comprising the nucleic acid sequence SEQ ID NO. 17701, preferably the coding region thereof, conferred the production of or the increase in isoleucine compared with the wild type control.

The nucleic acid sequence of At1g26830 from Arabidopsis thaliana, e.g. as shown in the respective line in column 5 of Table I, application no. 6, has been published in F. And the activity of the gene product thereof is the activity of cullin.

Accordingly, in one embodiment, the process of the present invention for producing leucine in a non-human organism, like a microorganism or a plant or a part thereof, comprises increasing or generating the activity of a gene product with the activity of a gene product conferring the activity of “cullin”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said At1g26830, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At1g26830, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At1g26830, or a functional equivalent or a homolog thereof as depicted in column 8 of Table II, application no. 6, preferably a homolog or functional equivalent as depicted in column 8 of Table II B, application no. 6, and being depicted in the same respective line as said At1g26830, and preferably the activity is increased non-targeted, whereby the respective line discloses in column 7 the fine chemical leucine.

Accordingly, in one embodiment, the molecule which activity is to be increased in the process of the invention is the gene product with an activity as a “cullin”, preferably it is the molecule of section (a) or (b) of this paragraph.

In particular, it was observed that in plants, especially in Arabidopsis thaliana, increasing or generating the activity of a gene product non-targeted with the activity of a “cullin”, preferably being encoded by a gene comprising the nucleic acid sequence SEQ ID NO. 17701, preferably the coding region thereof, conferred the production of or the increase in leucine compared with the wild type control.

The nucleic acid sequence of At1g36730 from Arabidopsis thaliana, e.g. as shown in the respective line in column 5 of Table I, application no. 6, has been published in F. And the activity of the gene product thereof is the activity of eukaryotic translation initiation factor 5.

Accordingly, in one embodiment, the process of the present invention for producing isoleucine in a non-human organism, like a microorganism or a plant or a part thereof, comprises increasing or generating the activity of a gene product with the activity of a gene product conferring the activity of “eukaryotic translation initiation factor 5”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said At1g36730, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At1g36730, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At1g36730, or a functional equivalent or a homolog thereof as depicted in column 8 of Table II, application no. 6, preferably a homolog or functional equivalent as depicted in column 8 of Table II B, application no. 6, and being depicted in the same respective line as said At1g36730, and preferably the activity is increased non-targeted, whereby the respective line discloses in column 7 the fine chemical isoleucine.

Accordingly, in one embodiment, the molecule which activity is to be increased in the process of the invention is the gene product with an activity as a “eukaryotic translation initiation factor 5”, preferably it is the molecule of section (a) or (b) of this paragraph.

In particular, it was observed that in plants, especially in Arabidopsis thaliana, increasing or generating the activity of a gene product non-targeted with the activity of a “eukaryotic translation initiation factor 5”, preferably being encoded by a gene comprising the nucleic acid sequence SEQ ID NO. 17968, preferably the coding region thereof, conferred the production of or the increase in isoleucine compared with the wild type control.

The nucleic acid sequence of At1g36730 from Arabidopsis thaliana, e.g. as shown in the respective line in column 5 of Table I, application no. 6, has been published in F. And the activity of the gene product thereof is the activity of eukaryotic translation initiation factor 5.

Accordingly, in one embodiment, the process of the present invention for producing leucine in a non-human organism, like a microorganism or a plant or a part thereof, comprises increasing or generating the activity of a gene product with the activity of a gene product conferring the activity of “eukaryotic translation initiation factor 5”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said At1g36730, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At1g36730, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At1g36730, or a functional equivalent or a homolog thereof as depicted in column 8 of Table II, application no. 6, preferably a homolog or functional equivalent as depicted in column 8 of Table II B, application no. 6, and being depicted in the same respective line as said At1g36730, and preferably the activity is increased non-targeted, whereby the respective line discloses in column 7 the fine chemical leucine.

Accordingly, in one embodiment, the molecule which activity is to be increased in the process of the invention is the gene product with an activity as a “eukaryotic translation initiation factor 5”, preferably it is the molecule of section (a) or (b) of this paragraph.

In particular, it was observed that in plants, especially in Arabidopsis thaliana, increasing or generating the activity of a gene product non-targeted with the activity of a “eukaryotic translation initiation factor 5”, preferably being encoded by a gene comprising the nucleic acid sequence SEQ ID NO. 17968, preferably the coding region thereof, conferred the production of or the increase in leucine compared with the wild type control.

The nucleic acid sequence of At1g43850 from Arabidopsis thaliana, e.g. as shown in the respective line in column 5 of Table I, application no. 6, has been published in F. And the activity of the gene product thereof is the activity of transcriptional regulator.

Accordingly, in one embodiment, the process of the present invention for producing isoleucine in a non-human organism, like a microorganism or a plant or a part thereof, comprises increasing or generating the activity of a gene product with the activity of a gene product conferring the activity of “transcriptional regulator”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said At1g43850, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At1g43850, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At1g43850, or a functional equivalent or a homolog thereof as depicted in column 8 of Table II, application no. 6, preferably a homolog or functional equivalent as depicted in column 8 of Table II B, application no. 6, and being depicted in the same respective line as said At1g43850, and preferably the activity is increased non-targeted, whereby the respective line discloses in column 7 the fine chemical isoleucine.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 18070, preferably the coding region thereof, conferred the production of or the increase in isoleucine compared with the wild type control.

The nucleic acid sequence of At1g43850 from Arabidopsis thaliana, e.g. as shown in the respective line in column 5 of Table I, application no. 6, has been published in F. And the activity of the gene product thereof is the activity of transcriptional regulator.

Accordingly, in one embodiment, the process of the present invention for producing leucine in a non-human organism, like a microorganism or a plant or a part thereof, comprises increasing or generating the activity of a gene product with the activity of a gene product conferring the activity of “transcriptional regulator”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said At1g43850, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At1g43850, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At1g43850, or a functional equivalent or a homolog thereof as depicted in column 8 of Table II, application no. 6, preferably a homolog or functional equivalent as depicted in column 8 of Table II B, application no. 6, and being depicted in the same respective line as said At1g43850, and preferably the activity is increased non-targeted, whereby the respective line discloses in column 7 the fine chemical leucine.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 18070, preferably the coding region thereof, conferred the production of or the increase in leucine compared with the wild type control.

The nucleic acid sequence of At1g43850 from Arabidopsis thaliana, e.g. as shown in the respective line in column 5 of Table I, application no. 6, has been published in F. And the activity of the gene product thereof is the activity of transcriptional regulator.

Accordingly, in one embodiment, the process of the present invention for producing valine in a non-human organism, like a microorganism or a plant or a part thereof, comprises increasing or generating the activity of a gene product with the activity of a gene product conferring the activity of “transcriptional regulator”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At1g43850, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At1g43850, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At1g43850, or a functional equivalent or a homolog thereof as depicted in column 8 of Table II, application no. 6, preferably a homolog or functional equivalent as depicted in column 8 of Table II B, application no. 6, and being depicted in the same respective line as said At1g43850, and preferably the activity is increased non-targeted, whereby the respective line discloses in column 7 the fine chemical valine.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 18070, preferably the coding region thereof, conferred the production of or the increase in valine compared with the wild type control.

The nucleic acid sequence of At1g47380 from Arabidopsis thaliana, e.g. as shown in the respective line in column 5 of Table I, application no. 6, has been published in F. And the activity of the gene product thereof is the activity of At1g47380-protein.

Accordingly, in one embodiment, the process of the present invention for producing leucine in a non-human organism, like a microorganism or a plant or a part thereof, comprises increasing or generating the activity of a gene product with the activity of a gene product conferring the activity of “At1g47380-protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said At1g47380, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At1g47380, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At1g47380, or a functional equivalent or a homolog thereof as depicted in column 8 of Table II, application no. 6, preferably a homolog or functional equivalent as depicted in column 8 of Table II B, application no. 6, and being depicted in the same respective line as said At1g47380, and preferably the activity is increased non-targeted, whereby the respective line discloses in column 7 the fine chemical leucine.

Accordingly, in one embodiment, the molecule which activity is to be increased in the process of the invention is the gene product with an activity as a “At1g47380-protein”, preferably it is the molecule of section (a) or (b) of this paragraph.

In particular, it was observed that in plants, especially in Arabidopsis thaliana, increasing or generating the activity of a gene product non-targeted with the activity of a “At1g47380-protein”, preferably being encoded by a gene comprising the nucleic acid sequence SEQ ID NO. 18122, preferably the coding region thereof, conferred the production of or the increase in leucine compared with the wild type control.

The nucleic acid sequence of At1g47380 from Arabidopsis thaliana, e.g. as shown in the respective line in column 5 of Table I, application no. 6, has been published in F. And the activity of the gene product thereof is the activity of At1g47380-protein.

Accordingly, in one embodiment, the process of the present invention for producing valine in a non-human organism, like a microorganism or a plant or a part thereof, comprises increasing or generating the activity of a gene product with the activity of a gene product conferring the activity of “At1g47380-protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At1g47380, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At1g47380, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At1g47380, or a functional equivalent or a homolog thereof as depicted in column 8 of Table II, application no. 6, preferably a homolog or functional equivalent as depicted in column 8 of Table II B, application no. 6, and being depicted in the same respective line as said At1g47380, and preferably the activity is increased non-targeted, whereby the respective line discloses in column 7 the fine chemical valine.

Accordingly, in one embodiment, the molecule which activity is to be increased in the process of the invention is the gene product with an activity as a “At1g47380-protein”, preferably it is the molecule of section (a) or (b) of this paragraph.

In particular, it was observed that in plants, especially in Arabidopsis thaliana, increasing or generating the activity of a gene product non-targeted with the activity of a “At1g47380-protein”, preferably being encoded by a gene comprising the nucleic acid sequence SEQ ID NO. 18122, preferably the coding region thereof, conferred the production of or the increase in valine compared with the wild type control.

The nucleic acid sequence of At1g48260 from Arabidopsis thaliana, e.g. as shown in the respective line in column 5 of Table I, application no. 6, has been published in F. And the activity of the gene product thereof is the activity of protein kinase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said At1g48260, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At1g48260, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At1g48260, or a functional equivalent or a homolog thereof as depicted in column 8 of Table II, application no. 6, preferably a homolog or functional equivalent as depicted in column 8 of Table II B, application no. 6, and being depicted in the same respective line as said At1g48260, and preferably the activity is increased non-targeted, whereby the respective line discloses in column 7 the fine chemical isoleucine.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 18235, preferably the coding region thereof, conferred the production of or the increase in isoleucine compared with the wild type control.

The nucleic acid sequence of At1g48260 from Arabidopsis thaliana, e.g. as shown in the respective line in column 5 of Table I, application no. 6, has been published in F. And the activity of the gene product thereof is the activity of protein kinase.

Accordingly, in one embodiment, the process of the present invention for producing leucine in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said At1g48260, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At1g48260, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At1g48260, or a functional equivalent or a homolog thereof as depicted in column 8 of Table II, application no. 6, preferably a homolog or functional equivalent as depicted in column 8 of Table II B, application no. 6, and being depicted in the same respective line as said At1g48260, and preferably the activity is increased non-targeted, whereby the respective line discloses in column 7 the fine chemical leucine.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 18235, preferably the coding region thereof, conferred the production of or the increase in leucine compared with the wild type control.

The nucleic acid sequence of At1g48260 from Arabidopsis thaliana, e.g. as shown in the respective line in column 5 of Table I, application no. 6, has been published in F. And the activity of the gene product thereof is the activity of protein kinase.

Accordingly, in one embodiment, the process of the present invention for producing valine in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At1g48260, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At1g48260, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At1g48260, or a functional equivalent or a homolog thereof as depicted in column 8 of Table II, application no. 6, preferably a homolog or functional equivalent as depicted in column 8 of Table II B, application no. 6, and being depicted in the same respective line as said At1g48260, and preferably the activity is increased non-targeted, whereby the respective line discloses in column 7 the fine chemical valine.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 18235, preferably the coding region thereof, conferred the production of or the increase in valine compared with the wild type control.

The nucleic acid sequence of At1g61950 from Arabidopsis thaliana, e.g. as shown in the respective line in column 5 of Table I, application no. 6, has been published in F. And the activity of the gene product thereof is the activity of calcium-dependent protein kinase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said At1g61950, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At1g61950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At1g61950, or a functional equivalent or a homolog thereof as depicted in column 8 of Table II, application no. 6, preferably a homolog or functional equivalent as depicted in column 8 of Table II B, application no. 6, and being depicted in the same respective line as said At1g61950, and preferably the activity is increased non-targeted, whereby the respective line discloses in column 7 the fine chemical isoleucine.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 18869, preferably the coding region thereof, conferred the production of or the increase in isoleucine compared with the wild type control.

The nucleic acid sequence of At1g61950 from Arabidopsis thaliana, e.g. as shown in the respective line in column 5 of Table I, application no. 6, has been published in F. And the activity of the gene product thereof is the activity of calcium-dependent protein kinase.

Accordingly, in one embodiment, the process of the present invention for producing leucine in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said At1g61950, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At1g61950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At1g61950, or a functional equivalent or a homolog thereof as depicted in column 8 of Table II, application no. 6, preferably a homolog or functional equivalent as depicted in column 8 of Table II B, application no. 6, and being depicted in the same respective line as said At1g61950, and preferably the activity is increased non-targeted, whereby the respective line discloses in column 7 the fine chemical leucine.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 18869, preferably the coding region thereof, conferred the production of or the increase in leucine compared with the wild type control.

The nucleic acid sequence of At1g61950 from Arabidopsis thaliana, e.g. as shown in the respective line in column 5 of Table I, application no. 6, has been published in F. And the activity of the gene product thereof is the activity of calcium-dependent protein kinase.

Accordingly, in one embodiment, the process of the present invention for producing valine in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At1g61950, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At1g61950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At1g61950, or a functional equivalent or a homolog thereof as depicted in column 8 of Table II, application no. 6, preferably a homolog or functional equivalent as depicted in column 8 of Table II B, application no. 6, and being depicted in the same respective line as said At1g61950, and preferably the activity is increased non-targeted, whereby the respective line discloses in column 7 the fine chemical valine.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 18869, preferably the coding region thereof, conferred the production of or the increase in valine compared with the wild type control.

The nucleic acid sequence of At1g68410 from Arabidopsis thaliana, e.g. as shown in the respective line in column 5 of Table I, application no. 6, has been published in F. And the activity of the gene product thereof is the activity of protein phosphatase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said At1g68410, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At1g68410, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At1g68410, or a functional equivalent or a homolog thereof as depicted in column 8 of Table II, application no. 6, preferably a homolog or functional equivalent as depicted in column 8 of Table II B, application no. 6, and being depicted in the same respective line as said At1g68410, and preferably the activity is increased non-targeted, whereby the respective line discloses in column 7 the fine chemical isoleucine.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 102119, preferably the coding region thereof, conferred the production of or the increase in isoleucine compared with the wild type control.

The nucleic acid sequence of At1g68410 from Arabidopsis thaliana, e.g. as shown in the respective line in column 5 of Table I, application no. 6, has been published in F. And the activity of the gene product thereof is the activity of protein phosphatase.

Accordingly, in one embodiment, the process of the present invention for producing leucine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said At1g68410, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At1g68410, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At1g68410, or a functional equivalent or a homolog thereof as depicted in column 8 of Table II, application no. 6, preferably a homolog or functional equivalent as depicted in column 8 of Table II B, application no. 6, and being depicted in the same respective line as said At1g68410, and preferably the activity is increased non-targeted, whereby the respective line discloses in column 7 the fine chemical leucine.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 102119, preferably the coding region thereof, conferred the production of or the increase in leucine compared with the wild type control.

The nucleic acid sequence of At1g72770 from Arabidopsis thaliana, e.g. as shown in the respective line in column 5 of Table I, application no. 6, has been published in F. And the activity of the gene product thereof is the activity of protein phosphatase.

Accordingly, in one embodiment, the process of the present invention for producing valine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At1g72770, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At1g72770, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At1g72770, or a functional equivalent or a homolog thereof as depicted in column 8 of Table II, application no. 6, preferably a homolog or functional equivalent as depicted in column 8 of Table II B, application no. 6, and being depicted in the same respective line as said At1g72770, and preferably the activity is increased non-targeted, whereby the respective line discloses in column 7 the fine chemical valine.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19419, preferably the coding region thereof, conferred the production of or the increase in valine compared with the wild type control.

The nucleic acid sequence of At2g17560 from Arabidopsis thaliana, e.g. as shown in the respective line in column 5 of Table I, application no. 6, has been published in F. And the activity of the gene product thereof is the activity of transcription factor.

Accordingly, in one embodiment, the process of the present invention for producing valine in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At2g17560, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At2g17560, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At2g17560, or a functional equivalent or a homolog thereof as depicted in column 8 of Table II, application no. 6, preferably a homolog or functional equivalent as depicted in column 8 of Table II B, application no. 6, and being depicted in the same respective line as said At2g17560, and preferably the activity is increased non-targeted, whereby the respective line discloses in column 7 the fine chemical valine.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 19502, preferably the coding region thereof, conferred the production of or the increase in valine compared with the wild type control.

The nucleic acid sequence of At2g28890 from Arabidopsis thaliana, e.g. as shown in the respective line in column 5 of Table I, application no. 6, has been published in F. And the activity of the gene product thereof is the activity of protein phosphatase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said At2g28890, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At2g28890, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At2g28890, or a functional equivalent or a homolog thereof as depicted in column 8 of Table II, application no. 6, preferably a homolog or functional equivalent as depicted in column 8 of Table II B, application no. 6, and being depicted in the same respective line as said At2g28890, and preferably the activity is increased non-targeted, whereby the respective line discloses in column 7 the fine chemical isoleucine.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19874, preferably the coding region thereof, conferred the production of or the increase in isoleucine compared with the wild type control.

The nucleic acid sequence of At2g28890 from Arabidopsis thaliana, e.g. as shown in the respective line in column 5 of Table I, application no. 6, has been published in F. And the activity of the gene product thereof is the activity of protein phosphatase.

Accordingly, in one embodiment, the process of the present invention for producing valine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At2g28890, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At2g28890, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At2g28890, or a functional equivalent or a homolog thereof as depicted in column 8 of Table II, application no. 6, preferably a homolog or functional equivalent as depicted in column 8 of Table II B, application no. 6, and being depicted in the same respective line as said At2g28890, and preferably the activity is increased non-targeted, whereby the respective line discloses in column 7 the fine chemical valine.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19874, preferably the coding region thereof, conferred the production of or the increase in valine compared with the wild type control.

The nucleic acid sequence of At2g30360 from Arabidopsis thaliana, e.g. as shown in the respective line in column 5 of Table I, application no. 6, has been published in F. And the activity of the gene product thereof is the activity of CBL-interacting protein kinase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “CBL-interacting protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said At2g30360, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At2g30360, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At2g30360, or a functional equivalent or a homolog thereof as depicted in column 8 of Table II, application no. 6, preferably a homolog or functional equivalent as depicted in column 8 of Table II B, application no. 6, and being depicted in the same respective line as said At2g30360, and preferably the activity is increased non-targeted, whereby the respective line discloses in column 7 the fine chemical isoleucine.

Accordingly, in one embodiment, the molecule which activity is to be increased in the process of the invention is the gene product with an activity as a “CBL-interacting protein kinase”, preferably it is the molecule of section (a) or (b) of this paragraph.

In particular, it was observed that in plants, especially in Arabidopsis thaliana, increasing or generating the activity of a gene product non-targeted with the activity of a “CBL-interacting protein kinase”, preferably being encoded by a gene comprising the nucleic acid sequence SEQ ID NO. 19919, preferably the coding region thereof, conferred the production of or the increase in isoleucine compared with the wild type control.

The nucleic acid sequence of At2g30360 from Arabidopsis thaliana, e.g. as shown in the respective line in column 5 of Table I, application no. 6, has been published in F. And the activity of the gene product thereof is the activity of CBL-interacting protein kinase.

Accordingly, in one embodiment, the process of the present invention for producing leucine in a non-human organism, like a microorganism or a plant or a part thereof, comprises increasing or generating the activity of a gene product with the activity of a gene product conferring the activity of “CBL-interacting protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said At2g30360, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At2g30360, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said

At2g30360, or a functional equivalent or a homolog thereof as depicted in column 8 of Table II, application no. 6, preferably a homolog or functional equivalent as depicted in column 8 of Table II B, application no. 6, and being depicted in the same respective line as said At2g30360, and preferably the activity is increased non-targeted, whereby the respective line discloses in column 7 the fine chemical leucine.

Accordingly, in one embodiment, the molecule which activity is to be increased in the process of the invention is the gene product with an activity as a “CBL-interacting protein kinase”, preferably it is the molecule of section (a) or (b) of this paragraph.

In particular, it was observed that in plants, especially in Arabidopsis thaliana, increasing or generating the activity of a gene product non-targeted with the activity of a “CBL-interacting protein kinase”, preferably being encoded by a gene comprising the nucleic acid sequence SEQ ID NO. 19919, preferably the coding region thereof, conferred the production of or the increase in leucine compared with the wild type control.

The nucleic acid sequence of At2g30360 from Arabidopsis thaliana, e.g. as shown in the respective line in column 5 of Table I, application no. 6, has been published in F. And the activity of the gene product thereof is the activity of CBL-interacting protein kinase.

Accordingly, in one embodiment, the process of the present invention for producing valine in a non-human organism, like a microorganism or a plant or a part thereof, comprises increasing or generating the activity of a gene product with the activity of a gene product conferring the activity of “CBL-interacting protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At2g30360, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At2g30360, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At2g30360, or a functional equivalent or a homolog thereof as depicted in column 8 of Table II, application no. 6, preferably a homolog or functional equivalent as depicted in column 8 of Table II B, application no. 6, and being depicted in the same respective line as said At2g30360, and preferably the activity is increased non-targeted, whereby the respective line discloses in column 7 the fine chemical valine.

Accordingly, in one embodiment, the molecule which activity is to be increased in the process of the invention is the gene product with an activity as a “CBL-interacting protein kinase”, preferably it is the molecule of section (a) or (b) of this paragraph.

In particular, it was observed that in plants, especially in Arabidopsis thaliana, increasing or generating the activity of a gene product non-targeted with the activity of a “CBL-interacting protein kinase”, preferably being encoded by a gene comprising the nucleic acid sequence SEQ ID NO. 19919, preferably the coding region thereof, conferred the production of or the increase in valine compared with the wild type control.

The nucleic acid sequence of At2g30540 from Arabidopsis thaliana, e.g. as shown in the respective line in column 5 of Table I, application no. 6, has been published in F. And the activity of the gene product thereof is the activity of glutaredoxin.

Accordingly, in one embodiment, the process of the present invention for producing isoleucine in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said At2g30540, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At2g30540, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At2g30540, or a functional equivalent or a homolog thereof as depicted in column 8 of Table II, application no. 6, preferably a homolog or functional equivalent as depicted in column 8 of Table II B, application no. 6, and being depicted in the same respective line as said At2g30540, and preferably the activity is increased non-targeted, whereby the respective line discloses in column 7 the fine chemical isoleucine.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 20346, preferably the coding region thereof, conferred the production of or the increase in isoleucine compared with the wild type control.

The nucleic acid sequence of At2g30540 from Arabidopsis thaliana, e.g. as shown in the respective line in column 5 of Table I, application no. 6, has been published in F. And the activity of the gene product thereof is the activity of glutaredoxin.

Accordingly, in one embodiment, the process of the present invention for producing leucine in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said At2g30540, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At2g30540, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At2g30540, or a functional equivalent or a homolog thereof as depicted in column 8 of Table II, application no. 6, preferably a homolog or functional equivalent as depicted in column 8 of Table II B, application no. 6, and being depicted in the same respective line as said At2g30540, and preferably the activity is increased non-targeted, whereby the respective line discloses in column 7 the fine chemical leucine.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 20346, preferably the coding region thereof, conferred the production of or the increase in leucine compared with the wild type control.

The nucleic acid sequence of At2g30540 from Arabidopsis thaliana, e.g. as shown in the respective line in column 5 of Table I, application no. 6, has been published in F. And the activity of the gene product thereof is the activity of glutaredoxin.

Accordingly, in one embodiment, the process of the present invention for producing valine in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At2g30540, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At2g30540, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At2g30540, or a functional equivalent or a homolog thereof as depicted in column 8 of Table II, application no. 6, preferably a homolog or functional equivalent as depicted in column 8 of Table II B, application no. 6, and being depicted in the same respective line as said At2g30540, and preferably the activity is increased non-targeted, whereby the respective line discloses in column 7 the fine chemical valine.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 20346, preferably the coding region thereof, conferred the production of or the increase in valine compared with the wild type control.

The nucleic acid sequence of At2g47880 from Arabidopsis thaliana, e.g. as shown in the respective line in column 5 of Table I, application no. 6, has been published in F. And the activity of the gene product thereof is the activity of glutaredoxin.

Accordingly, in one embodiment, the process of the present invention for producing valine in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At2g47880, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At2g47880, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At2g47880, or a functional equivalent or a homolog thereof as depicted in column 8 of Table II, application no. 6, preferably a homolog or functional equivalent as depicted in column 8 of Table II B, application no. 6, and being depicted in the same respective line as said At2g47880, and preferably the activity is increased non-targeted, whereby the respective line discloses in column 7 the fine chemical valine.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 21159, preferably the coding region thereof, conferred the production of or the increase in valine compared with the wild type control.

The nucleic acid sequence of At3g04050 from Arabidopsis thaliana, e.g. as shown in the respective line in column 5 of Table I, application no. 6, has been published in F. And the activity of the gene product thereof is the activity of pyruvate kinase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “pyruvate kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said At3g04050, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At3g04050, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At3g04050, or a functional equivalent or a homolog thereof as depicted in column 8 of Table II, application no. 6, preferably a homolog or functional equivalent as depicted in column 8 of Table II B, application no. 6, and being depicted in the same respective line as said At3g04050, and preferably the activity is increased plastidic, whereby the respective line discloses in column 7 the fine chemical isoleucine.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 21497, preferably the coding region thereof, conferred the production of or the increase in isoleucine compared with the wild type control.

The nucleic acid sequence of At3g04050 from Arabidopsis thaliana, e.g. as shown in the respective line in column 5 of Table I, application no. 6, has been published in F. And the activity of the gene product thereof is the activity of pyruvate kinase.

Accordingly, in one embodiment, the process of the present invention for producing leucine in a non-human organism, like a microorganism or a plant or a part thereof, comprises increasing or generating the activity of a gene product with the activity of a gene product conferring the activity of “pyruvate kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said At3g04050, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At3g04050, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At3g04050, or a functional equivalent or a homolog thereof as depicted in column 8 of Table II, application no. 6, preferably a homolog or functional equivalent as depicted in column 8 of Table II B, application no. 6, and being depicted in the same respective line as said At3g04050, and preferably the activity is increased plastidic, whereby the respective line discloses in column 7 the fine chemical leucine.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 21497, preferably the coding region thereof, conferred the production of or the increase in leucine compared with the wild type control.

The nucleic acid sequence of At3g04050 from Arabidopsis thaliana, e.g. as shown in the respective line in column 5 of Table I, application no. 6, has been published in F. And the activity of the gene product thereof is the activity of pyruvate kinase.

Accordingly, in one embodiment, the process of the present invention for producing valine in a non-human organism, like a microorganism or a plant or a part thereof, comprises increasing or generating the activity of a gene product with the activity of a gene product conferring the activity of “pyruvate kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At3g04050, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At3g04050, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At3g04050, or a functional equivalent or a homolog thereof as depicted in column 8 of Table II, application no. 6, preferably a homolog or functional equivalent as depicted in column 8 of Table II B, application no. 6, and being depicted in the same respective line as said At3g04050, and preferably the activity is increased plastidic, whereby the respective line discloses in column 7 the fine chemical valine.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 21497, preferably the coding region thereof, conferred the production of or the increase in valine compared with the wild type control.

The nucleic acid sequence of At3g08710 from Arabidopsis thaliana, e.g. as shown in the respective line in column 5 of Table I, application no. 6, has been published in F. And the activity of the gene product thereof is the activity of thioredoxin family protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “thioredoxin family protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said At3g08710, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At3g08710, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said

At3g08710, or a functional equivalent or a homolog thereof as depicted in column 8 of Table II, application no. 6, preferably a homolog or functional equivalent as depicted in column 8 of Table II B, application no. 6, and being depicted in the same respective line as said At3g08710, and preferably the activity is increased non-targeted, whereby the respective line discloses in column 7 the fine chemical isoleucine.

Accordingly, in one embodiment, the molecule which activity is to be increased in the process of the invention is the gene product with an activity as a “thioredoxin family protein”, preferably it is the molecule of section (a) or (b) of this paragraph.

In particular, it was observed that in plants, especially in Arabidopsis thaliana, increasing or generating the activity of a gene product non-targeted with the activity of a “thioredoxin family protein”, preferably being encoded by a gene comprising the nucleic acid sequence SEQ ID NO. 22249, preferably the coding region thereof, conferred the production of or the increase in isoleucine compared with the wild type control.

The nucleic acid sequence of At3g08710 from Arabidopsis thaliana, e.g. as shown in the respective line in column 5 of Table I, application no. 6, has been published in F. And the activity of the gene product thereof is the activity of thioredoxin family protein.

Accordingly, in one embodiment, the process of the present invention for producing leucine in a non-human organism, like a microorganism or a plant or a part thereof, comprises increasing or generating the activity of a gene product with the activity of a gene product conferring the activity of “thioredoxin family protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said At3g08710, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At3g08710, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At3g08710, or a functional equivalent or a homolog thereof as depicted in column 8 of Table II, application no. 6, preferably a homolog or functional equivalent as depicted in column 8 of Table II B, application no. 6, and being depicted in the same respective line as said At3g08710, and preferably the activity is increased non-targeted, whereby the respective line discloses in column 7 the fine chemical leucine.

Accordingly, in one embodiment, the molecule which activity is to be increased in the process of the invention is the gene product with an activity as a “thioredoxin family protein”, preferably it is the molecule of section (a) or (b) of this paragraph.

In particular, it was observed that in plants, especially in Arabidopsis thaliana, increasing or generating the activity of a gene product non-targeted with the activity of a “thioredoxin family protein”, preferably being encoded by a gene comprising the nucleic acid sequence SEQ ID NO. 22249, preferably the coding region thereof, conferred the production of or the increase in leucine compared with the wild type control.

The nucleic acid sequence of At3g11650 from Arabidopsis thaliana, e.g. as shown in the respective line in column 5 of Table I, application no. 6, has been published in F. And the activity of the gene product thereof is the activity of harpin-induced family protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “harpin-induced family protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said At3g11650, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At3g11650, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At3g11650, or a functional equivalent or a homolog thereof as depicted in column 8 of Table II, application no. 6, preferably a homolog or functional equivalent as depicted in column 8 of Table II B, application no. 6, and being depicted in the same respective line as said At3g11650, and preferably the activity is increased non-targeted, whereby the respective line discloses in column 7 the fine chemical isoleucine.

Accordingly, in one embodiment, the molecule which activity is to be increased in the process of the invention is the gene product with an activity as a “harpin-induced family protein”, preferably it is the molecule of section (a) or (b) of this paragraph.

In particular, it was observed that in plants, especially in Arabidopsis thaliana, increasing or generating the activity of a gene product non-targeted with the activity of a “harpin-induced family protein”, preferably being encoded by a gene comprising the nucleic acid sequence SEQ ID NO. 22611, preferably the coding region thereof, conferred the production of or the increase in isoleucine compared with the wild type control.

The nucleic acid sequence of At3g11650 from Arabidopsis thaliana, e.g. as shown in the respective line in column 5 of Table I, application no. 6, has been published in F. And the activity of the gene product thereof is the activity of harpin-induced family protein.

Accordingly, in one embodiment, the process of the present invention for producing leucine in a non-human organism, like a microorganism or a plant or a part thereof, comprises increasing or generating the activity of a gene product with the activity of a gene product conferring the activity of “harpin-induced family protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said At3g11650, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At3g11650, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At3g11650, or a functional equivalent or a homolog thereof as depicted in column 8 of Table II, application no. 6, preferably a homolog or functional equivalent as depicted in column 8 of Table II B, application no. 6, and being depicted in the same respective line as said At3g11650, and preferably the activity is increased non-targeted, whereby the respective line discloses in column 7 the fine chemical leucine.

Accordingly, in one embodiment, the molecule which activity is to be increased in the process of the invention is the gene product with an activity as a “harpin-induced family protein”, preferably it is the molecule of section (a) or (b) of this paragraph.

In particular, it was observed that in plants, especially in Arabidopsis thaliana, increasing or generating the activity of a gene product non-targeted with the activity of a “harpin-induced family protein”, preferably being encoded by a gene comprising the nucleic acid sequence SEQ ID NO. 22611, preferably the coding region thereof, conferred the production of or the increase in leucine compared with the wild type control.

The nucleic acid sequence of At3g18524 from Arabidopsis thaliana, e.g. as shown in the respective line in column 5 of Table I, application no. 6, has been published in F. And the activity of the gene product thereof is the activity of DNA mismatch repair protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “DNA mismatch repair protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said At3g18524, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At3g18524, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At3g18524, or a functional equivalent or a homolog thereof as depicted in column 8 of Table II, application no. 6, preferably a homolog or functional equivalent as depicted in column 8 of Table II B, application no. 6, and being depicted in the same respective line as said At3g18524, and preferably the activity is increased non-targeted, whereby the respective line discloses in column 7 the fine chemical isoleucine.

Accordingly, in one embodiment, the molecule which activity is to be increased in the process of the invention is the gene product with an activity as a “DNA mismatch repair protein”, preferably it is the molecule of section (a) or (b) of this paragraph.

In particular, it was observed that in plants, especially in Arabidopsis thaliana, increasing or generating the activity of a gene product non-targeted with the activity of a “DNA mismatch repair protein”, preferably being encoded by a gene comprising the nucleic acid sequence SEQ ID NO. 22832, preferably the coding region thereof, conferred the production of or the increase in isoleucine compared with the wild type control.

The nucleic acid sequence of At3g18524 from Arabidopsis thaliana, e.g. as shown in the respective line in column 5 of Table I, application no. 6, has been published in F. And the activity of the gene product thereof is the activity of DNA mismatch repair protein.

Accordingly, in one embodiment, the process of the present invention for producing leucine in a non-human organism, like a microorganism or a plant or a part thereof, comprises increasing or generating the activity of a gene product with the activity of a gene product conferring the activity of “DNA mismatch repair protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said At3g18524, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At3g18524, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At3g18524, or a functional equivalent or a homolog thereof as depicted in column 8 of Table II, application no. 6, preferably a homolog or functional equivalent as depicted in column 8 of Table II B, application no. 6, and being depicted in the same respective line as said At3g18524, and preferably the activity is increased non-targeted, whereby the respective line discloses in column 7 the fine chemical leucine.

Accordingly, in one embodiment, the molecule which activity is to be increased in the process of the invention is the gene product with an activity as a “DNA mismatch repair protein”, preferably it is the molecule of section (a) or (b) of this paragraph.

In particular, it was observed that in plants, especially in Arabidopsis thaliana, increasing or generating the activity of a gene product non-targeted with the activity of a “DNA mismatch repair protein”, preferably being encoded by a gene comprising the nucleic acid sequence SEQ ID NO. 22832, preferably the coding region thereof, conferred the production of or the increase in leucine compared with the wild type control.

The nucleic acid sequence of At3g18524 from Arabidopsis thaliana, e.g. as shown in the respective line in column 5 of Table I, application no. 6, has been published in F. And the activity of the gene product thereof is the activity of DNA mismatch repair protein.

Accordingly, in one embodiment, the process of the present invention for producing valine in a non-human organism, like a microorganism or a plant or a part thereof, comprises increasing or generating the activity of a gene product with the activity of a gene product conferring the activity of “DNA mismatch repair protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At3g18524, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At3g18524, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At3g18524, or a functional equivalent or a homolog thereof as depicted in column 8 of Table II, application no. 6, preferably a homolog or functional equivalent as depicted in column 8 of Table II B, application no. 6, and being depicted in the same respective line as said At3g18524, and preferably the activity is increased non-targeted, whereby the respective line discloses in column 7 the fine chemical valine.

Accordingly, in one embodiment, the molecule which activity is to be increased in the process of the invention is the gene product with an activity as a “DNA mismatch repair protein”, preferably it is the molecule of section (a) or (b) of this paragraph.

In particular, it was observed that in plants, especially in Arabidopsis thaliana, increasing or generating the activity of a gene product non-targeted with the activity of a “DNA mismatch repair protein”, preferably being encoded by a gene comprising the nucleic acid sequence SEQ ID NO. 22832, preferably the coding region thereof, conferred the production of or the increase in valine compared with the wild type control.

The nucleic acid sequence of At3g23000 from Arabidopsis thaliana, e.g. as shown in the respective line in column 5 of Table I, application no. 6, has been published in F. And the activity of the gene product thereof is the activity of protein kinase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said At3g23000, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At3g23000, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At3g23000, or a functional equivalent or a homolog thereof as depicted in column 8 of Table II, application no. 6, preferably a homolog or functional equivalent as depicted in column 8 of Table II B, application no. 6, and being depicted in the same respective line as said At3g23000, and preferably the activity is increased non-targeted, whereby the respective line discloses in column 7 the fine chemical isoleucine.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 1815, preferably the coding region thereof, conferred the production of or the increase in isoleucine compared with the wild type control.

The nucleic acid sequence of At3g23000 from Arabidopsis thaliana, e.g. as shown in the respective line in column 5 of Table I, application no. 6, has been published in F. And the activity of the gene product thereof is the activity of protein kinase.

Accordingly, in one embodiment, the process of the present invention for producing leucine in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said At3g23000, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At3g23000, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At3g23000, or a functional equivalent or a homolog thereof as depicted in column 8 of Table II, application no. 6, preferably a homolog or functional equivalent as depicted in column 8 of Table II B, application no. 6, and being depicted in the same respective line as said At3g23000, and preferably the activity is increased non-targeted, whereby the respective line discloses in column 7 the fine chemical leucine.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 1815, preferably the coding region thereof, conferred the production of or the increase in leucine compared with the wild type control.

The nucleic acid sequence of At3g23000 from Arabidopsis thaliana, e.g. as shown in the respective line in column 5 of Table I, application no. 6, has been published in F. And the activity of the gene product thereof is the activity of protein kinase.

Accordingly, in one embodiment, the process of the present invention for producing valine in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At3g23000, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At3g23000, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said

At3g23000, or a functional equivalent or a homolog thereof as depicted in column 8 of Table II, application no. 6, preferably a homolog or functional equivalent as depicted in column 8 of Table II B, application no. 6, and being depicted in the same respective line as said At3g23000, and preferably the activity is increased non-targeted, whereby the respective line discloses in column 7 the fine chemical valine.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 1815, preferably the coding region thereof, conferred the production of or the increase in valine compared with the wild type control.

The nucleic acid sequence of At3g26400 from Arabidopsis thaliana, e.g. as shown in the respective line in column 5 of Table I, application no. 6, has been published in F. And the activity of the gene product thereof is the activity of eukaryotic translation initiation factor.

Accordingly, in one embodiment, the process of the present invention for producing isoleucine in a non-human organism, like a microorganism or a plant or a part thereof, comprises increasing or generating the activity of a gene product with the activity of a gene product conferring the activity of “eukaryotic translation initiation factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said At3g26400, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At3g26400, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At3g26400, or a functional equivalent or a homolog thereof as depicted in column 8 of Table II, application no. 6, preferably a homolog or functional equivalent as depicted in column 8 of Table II B, application no. 6, and being depicted in the same respective line as said At3g26400, and preferably the activity is increased non-targeted, whereby the respective line discloses in column 7 the fine chemical isoleucine.

Accordingly, in one embodiment, the molecule which activity is to be increased in the process of the invention is the gene product with an activity as a “eukaryotic translation initiation factor”, preferably it is the molecule of section (a) or (b) of this paragraph.

In particular, it was observed that in plants, especially in Arabidopsis thaliana, increasing or generating the activity of a gene product non-targeted with the activity of a “eukaryotic translation initiation factor”, preferably being encoded by a gene comprising the nucleic acid sequence SEQ ID NO. 68727, preferably the coding region thereof, conferred the production of or the increase in isoleucine compared with the wild type control.

The nucleic acid sequence of At3g26400 from Arabidopsis thaliana, e.g. as shown in the respective line in column 5 of Table I, application no. 6, has been published in F. And the activity of the gene product thereof is the activity of eukaryotic translation initiation factor.

Accordingly, in one embodiment, the process of the present invention for producing leucine in a non-human organism, like a microorganism or a plant or a part thereof, comprises increasing or generating the activity of a gene product with the activity of a gene product conferring the activity of “eukaryotic translation initiation factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said At3g26400, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At3g26400, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At3g26400, or a functional equivalent or a homolog thereof as depicted in column 8 of Table II, application no. 6, preferably a homolog or functional equivalent as depicted in column 8 of Table II B, application no. 6, and being depicted in the same respective line as said At3g26400, and preferably the activity is increased non-targeted, whereby the respective line discloses in column 7 the fine chemical leucine.

Accordingly, in one embodiment, the molecule which activity is to be increased in the process of the invention is the gene product with an activity as a “eukaryotic translation initiation factor”, preferably it is the molecule of section (a) or (b) of this paragraph.

In particular, it was observed that in plants, especially in Arabidopsis thaliana, increasing or generating the activity of a gene product non-targeted with the activity of a “eukaryotic translation initiation factor”, preferably being encoded by a gene comprising the nucleic acid sequence SEQ ID NO. 68727, preferably the coding region thereof, conferred the production of or the increase in leucine compared with the wild type control.

The nucleic acid sequence of At3g26400 from Arabidopsis thaliana, e.g. as shown in the respective line in column 5 of Table I, application no. 6, has been published in F. And the activity of the gene product thereof is the activity of eukaryotic translation initiation factor.

Accordingly, in one embodiment, the process of the present invention for producing valine in a non-human organism, like a microorganism or a plant or a part thereof, comprises increasing or generating the activity of a gene product with the activity of a gene product conferring the activity of “eukaryotic translation initiation factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At3g26400, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At3g26400, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At3g26400, or a functional equivalent or a homolog thereof as depicted in column 8 of Table II, application no. 6, preferably a homolog or functional equivalent as depicted in column 8 of Table II B, application no. 6, and being depicted in the same respective line as said At3g26400, and preferably the activity is increased non-targeted, whereby the respective line discloses in column 7 the fine chemical valine.

Accordingly, in one embodiment, the molecule which activity is to be increased in the process of the invention is the gene product with an activity as a “eukaryotic translation initiation factor”, preferably it is the molecule of section (a) or (b) of this paragraph.

In particular, it was observed that in plants, especially in Arabidopsis thaliana, increasing or generating the activity of a gene product non-targeted with the activity of a “eukaryotic translation initiation factor”, preferably being encoded by a gene comprising the nucleic acid sequence SEQ ID NO. 68727, preferably the coding region thereof, conferred the production of or the increase in valine compared with the wild type control.

The nucleic acid sequence of At3g27540 from Arabidopsis thaliana, e.g. as shown in the respective line in column 5 of Table I, application no. 6, has been published in F. And the activity of the gene product thereof is the activity of glycosyl transferase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “glycosyl transferase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said At3g27540, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At3g27540, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At3g27540, or a functional equivalent or a homolog thereof as depicted in column 8 of Table II, application no. 6, preferably a homolog or functional equivalent as depicted in column 8 of Table II B, application no. 6, and being depicted in the same respective line as said At3g27540, and preferably the activity is increased non-targeted, whereby the respective line discloses in column 7 the fine chemical isoleucine.

Accordingly, in one embodiment, the molecule which activity is to be increased in the process of the invention is the gene product with an activity as a “glycosyl transferase”, preferably it is the molecule of section (a) or (b) of this paragraph.

In particular, it was observed that in plants, especially in Arabidopsis thaliana, increasing or generating the activity of a gene product non-targeted with the activity of a “glycosyl transferase”, preferably being encoded by a gene comprising the nucleic acid sequence SEQ ID NO. 68777, preferably the coding region thereof, conferred the production of or the increase in isoleucine compared with the wild type control.

The nucleic acid sequence of At3g27540 from Arabidopsis thaliana, e.g. as shown in the respective line in column 5 of Table I, application no. 6, has been published in F. And the activity of the gene product thereof is the activity of glycosyl transferase.

Accordingly, in one embodiment, the process of the present invention for producing leucine in a non-human organism, like a microorganism or a plant or a part thereof, comprises increasing or generating the activity of a gene product with the activity of a gene product conferring the activity of “glycosyl transferase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said At3g27540, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At3g27540, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At3g27540, or a functional equivalent or a homolog thereof as depicted in column 8 of Table II, application no. 6, preferably a homolog or functional equivalent as depicted in column 8 of Table II B, application no. 6, and being depicted in the same respective line as said At3g27540, and preferably the activity is increased non-targeted, whereby the respective line discloses in column 7 the fine chemical leucine.

Accordingly, in one embodiment, the molecule which activity is to be increased in the process of the invention is the gene product with an activity as a “glycosyl transferase”, preferably it is the molecule of section (a) or (b) of this paragraph.

In particular, it was observed that in plants, especially in Arabidopsis thaliana, increasing or generating the activity of a gene product non-targeted with the activity of a “glycosyl transferase”, preferably being encoded by a gene comprising the nucleic acid sequence SEQ ID NO. 68777, preferably the coding region thereof, conferred the production of or the increase in leucine compared with the wild type control.

The nucleic acid sequence of At3g27540 from Arabidopsis thaliana, e.g. as shown in the respective line in column 5 of Table I, application no. 6, has been published in F. And the activity of the gene product thereof is the activity of glycosyl transferase.

Accordingly, in one embodiment, the process of the present invention for producing valine in a non-human organism, like a microorganism or a plant or a part thereof, comprises increasing or generating the activity of a gene product with the activity of a gene product conferring the activity of “glycosyl transferase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At3g27540, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At3g27540, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At3g27540, or a functional equivalent or a homolog thereof as depicted in column 8 of Table II, application no. 6, preferably a homolog or functional equivalent as depicted in column 8 of Table II B, application no. 6, and being depicted in the same respective line as said At3g27540, and preferably the activity is increased non-targeted, whereby the respective line discloses in column 7 the fine chemical valine.

Accordingly, in one embodiment, the molecule which activity is to be increased in the process of the invention is the gene product with an activity as a “glycosyl transferase”, preferably it is the molecule of section (a) or (b) of this paragraph.

In particular, it was observed that in plants, especially in Arabidopsis thaliana, increasing or generating the activity of a gene product non-targeted with the activity of a “glycosyl transferase”, preferably being encoded by a gene comprising the nucleic acid sequence SEQ ID NO. 68777, preferably the coding region thereof, conferred the production of or the increase in valine compared with the wild type control.

The nucleic acid sequence of At3g61830 from Arabidopsis thaliana, e.g. as shown in the respective line in column 5 of Table I, application no. 6, has been published in F. And the activity of the gene product thereof is the activity of auxin response factor.

Accordingly, in one embodiment, the process of the present invention for producing isoleucine in a non-human organism, like a microorganism or a plant or a part thereof, comprises increasing or generating the activity of a gene product with the activity of a gene product conferring the activity of “auxin response factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said At3g61830, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At3g61830, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At3g61830, or a functional equivalent or a homolog thereof as depicted in column 8 of Table II, application no. 6, preferably a homolog or functional equivalent as depicted in column 8 of Table II B, application no. 6, and being depicted in the same respective line as said At3g61830, and preferably the activity is increased non-targeted, whereby the respective line discloses in column 7 the fine chemical isoleucine.

Accordingly, in one embodiment, the molecule which activity is to be increased in the process of the invention is the gene product with an activity as a “auxin response factor”, preferably it is the molecule of section (a) or (b) of this paragraph.

In particular, it was observed that in plants, especially in Arabidopsis thaliana, increasing or generating the activity of a gene product non-targeted with the activity of a “auxin response factor”, preferably being encoded by a gene comprising the nucleic acid sequence SEQ ID NO. 2367, preferably the coding region thereof, conferred the production of or the increase in isoleucine compared with the wild type control.

The nucleic acid sequence of At3g61830 from Arabidopsis thaliana, e.g. as shown in the respective line in column 5 of Table I, application no. 6, has been published in F. And the activity of the gene product thereof is the activity of auxin response factor.

Accordingly, in one embodiment, the process of the present invention for producing leucine in a non-human organism, like a microorganism or a plant or a part thereof, comprises increasing or generating the activity of a gene product with the activity of a gene product conferring the activity of “auxin response factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said At3g61830, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At3g61830, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At3g61830, or a functional equivalent or a homolog thereof as depicted in column 8 of Table II, application no. 6, preferably a homolog or functional equivalent as depicted in column 8 of Table II B, application no. 6, and being depicted in the same respective line as said At3g61830, and preferably the activity is increased non-targeted, whereby the respective line discloses in column 7 the fine chemical leucine.

Accordingly, in one embodiment, the molecule which activity is to be increased in the process of the invention is the gene product with an activity as a “auxin response factor”, preferably it is the molecule of section (a) or (b) of this paragraph.

In particular, it was observed that in plants, especially in Arabidopsis thaliana, increasing or generating the activity of a gene product non-targeted with the activity of a “auxin response factor”, preferably being encoded by a gene comprising the nucleic acid sequence SEQ ID NO. 2367, preferably the coding region thereof, conferred the production of or the increase in leucine compared with the wild type control.

The nucleic acid sequence of At3g61830 from Arabidopsis thaliana, e.g. as shown in the respective line in column 5 of Table I, application no. 6, has been published in F. And the activity of the gene product thereof is the activity of auxin response factor.

Accordingly, in one embodiment, the process of the present invention for producing valine in a non-human organism, like a microorganism or a plant or a part thereof, comprises increasing or generating the activity of a gene product with the activity of a gene product conferring the activity of “auxin response factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At3g61830, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At3g61830, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At3g61830, or a functional equivalent or a homolog thereof as depicted in column 8 of Table II, application no. 6, preferably a homolog or functional equivalent as depicted in column 8 of Table II B, application no. 6, and being depicted in the same respective line as said At3g61830, and preferably the activity is increased non-targeted, whereby the respective line discloses in column 7 the fine chemical valine.

Accordingly, in one embodiment, the molecule which activity is to be increased in the process of the invention is the gene product with an activity as a “auxin response factor”, preferably it is the molecule of section (a) or (b) of this paragraph.

In particular, it was observed that in plants, especially in Arabidopsis thaliana, increasing or generating the activity of a gene product non-targeted with the activity of a “auxin response factor”, preferably being encoded by a gene comprising the nucleic acid sequence SEQ ID NO. 2367, preferably the coding region thereof, conferred the production of or the increase in valine compared with the wild type control.

The nucleic acid sequence of At3g62950 from Arabidopsis thaliana, e.g. as shown in the respective line in column 5 of Table I, application no. 6, has been published in F. And the activity of the gene product thereof is the activity of glutaredoxin.

Accordingly, in one embodiment, the process of the present invention for producing isoleucine in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said At3g62950, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At3g62950, or a functional equivalent or a homolog thereof as depicted in column 8 of Table II, application no. 6, preferably a homolog or functional equivalent as depicted in column 8 of Table II B, application no. 6, and being depicted in the same respective line as said At3g62950, and preferably the activity is increased non-targeted, whereby the respective line discloses in column 7 the fine chemical isoleucine.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in isoleucine compared with the wild type control.

The nucleic acid sequence of At3g62950 from Arabidopsis thaliana, e.g. as shown in the respective line in column 5 of Table I, application no. 6, has been published in F. And the activity of the gene product thereof is the activity of glutaredoxin.

Accordingly, in one embodiment, the process of the present invention for producing leucine in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said At3g62950, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At3g62950, or a functional equivalent or a homolog thereof as depicted in column 8 of Table II, application no. 6, preferably a homolog or functional equivalent as depicted in column 8 of Table II B, application no. 6, and being depicted in the same respective line as said At3g62950, and preferably the activity is increased non-targeted, whereby the respective line discloses in column 7 the fine chemical leucine.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in leucine compared with the wild type control.

The nucleic acid sequence of At3g62950 from Arabidopsis thaliana, e.g. as shown in the respective line in column 5 of Table I, application no. 6, has been published in F. And the activity of the gene product thereof is the activity of glutaredoxin.

Accordingly, in one embodiment, the process of the present invention for producing valine in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At3g62950, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At3g62950, or a functional equivalent or a homolog thereof as depicted in column 8 of Table II, application no. 6, preferably a homolog or functional equivalent as depicted in column 8 of Table II B, application no. 6, and being depicted in the same respective line as said At3g62950, and preferably the activity is increased non-targeted, whereby the respective line discloses in column 7 the fine chemical valine.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in valine compared with the wild type control.

The nucleic acid sequence of At4g18880 from Arabidopsis thaliana, e.g. as shown in the respective line in column 5 of Table I, application no. 6, has been published in F. And the activity of the gene product thereof is the activity of heat shock transcription factor.

Accordingly, in one embodiment, the process of the present invention for producing isoleucine in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said At4g18880, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At4g18880, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At4g18880, or a functional equivalent or a homolog thereof as depicted in column 8 of Table II, application no. 6, preferably a homolog or functional equivalent as depicted in column 8 of Table II B, application no. 6, and being depicted in the same respective line as said At4g18880, and preferably the activity is increased non-targeted, whereby the respective line discloses in column 7 the fine chemical isoleucine.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 24232, preferably the coding region thereof, conferred the production of or the increase in isoleucine compared with the wild type control.

The nucleic acid sequence of At4g26080 from Arabidopsis thaliana, e.g. as shown in the respective line in column 5 of Table I, application no. 6, has been published in F. And the activity of the gene product thereof is the activity of protein phosphatase.

Accordingly, in one embodiment, the process of the present invention for producing valine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At4g26080, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At4g26080, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At4g26080, or a functional equivalent or a homolog thereof as depicted in column 8 of Table II, application no. 6, preferably a homolog or functional equivalent as depicted in column 8 of Table II B, application no. 6, and being depicted in the same respective line as said At4g26080, and preferably the activity is increased non-targeted, whereby the respective line discloses in column 7 the fine chemical valine.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 68849, preferably the coding region thereof, conferred the production of or the increase in valine compared with the wild type control.

The nucleic acid sequence of At4g32480 from Arabidopsis thaliana, e.g. as shown in the respective line in column 5 of Table I, application no. 6, has been published in F. And the activity of the gene product thereof is the activity of At4g32480-protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “At4g32480-protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said At4g32480, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At4g32480, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At4g32480, or a functional equivalent or a homolog thereof as depicted in column 8 of Table II, application no. 6, preferably a homolog or functional equivalent as depicted in column 8 of Table II B, application no. 6, and being depicted in the same respective line as said At4g32480, and preferably the activity is increased non-targeted, whereby the respective line discloses in column 7 the fine chemical isoleucine.

Accordingly, in one embodiment, the molecule which activity is to be increased in the process of the invention is the gene product with an activity as a “At4g32480-protein”, preferably it is the molecule of section (a) or (b) of this paragraph.

In particular, it was observed that in plants, especially in Arabidopsis thaliana, increasing or generating the activity of a gene product non-targeted with the activity of a “At4g32480-protein”, preferably being encoded by a gene comprising the nucleic acid sequence SEQ ID NO. 4040, preferably the coding region thereof, conferred the production of or the increase in isoleucine compared with the wild type control.

The nucleic acid sequence of At4g32480 from Arabidopsis thaliana, e.g. as shown in the respective line in column 5 of Table I, application no. 6, has been published in F. And the activity of the gene product thereof is the activity of At4g32480-protein.

Accordingly, in one embodiment, the process of the present invention for producing leucine in a non-human organism, like a microorganism or a plant or a part thereof, comprises increasing or generating the activity of a gene product with the activity of a gene product conferring the activity of “At4g32480-protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said At4g32480, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At4g32480, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At4g32480, or a functional equivalent or a homolog thereof as depicted in column 8 of Table II, application no. 6, preferably a homolog or functional equivalent as depicted in column 8 of Table II B, application no. 6, and being depicted in the same respective line as said At4g32480, and preferably the activity is increased non-targeted, whereby the respective line discloses in column 7 the fine chemical leucine.

Accordingly, in one embodiment, the molecule which activity is to be increased in the process of the invention is the gene product with an activity as a “At4g32480-protein”, preferably it is the molecule of section (a) or (b) of this paragraph.

In particular, it was observed that in plants, especially in Arabidopsis thaliana, increasing or generating the activity of a gene product non-targeted with the activity of a “At4g32480-protein”, preferably being encoded by a gene comprising the nucleic acid sequence SEQ ID NO. 4040, preferably the coding region thereof, conferred the production of or the increase in leucine compared with the wild type control.

The nucleic acid sequence of At4g32480 from Arabidopsis thaliana, e.g. as shown in the respective line in column 5 of Table I, application no. 6, has been published in F. And the activity of the gene product thereof is the activity of At4g32480-protein.

Accordingly, in one embodiment, the process of the present invention for producing valine in a non-human organism, like a microorganism or a plant or a part thereof, comprises increasing or generating the activity of a gene product with the activity of a gene product conferring the activity of “At4g32480-protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At4g32480, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At4g32480, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At4g32480, or a functional equivalent or a homolog thereof as depicted in column 8 of Table II, application no. 6, preferably a homolog or functional equivalent as depicted in column 8 of Table II B, application no. 6, and being depicted in the same respective line as said At4g32480, and preferably the activity is increased non-targeted, whereby the respective line discloses in column 7 the fine chemical valine.

Accordingly, in one embodiment, the molecule which activity is to be increased in the process of the invention is the gene product with an activity as a “At4g32480-protein”, preferably it is the molecule of section (a) or (b) of this paragraph.

In particular, it was observed that in plants, especially in Arabidopsis thaliana, increasing or generating the activity of a gene product non-targeted with the activity of a “At4g32480-protein”, preferably being encoded by a gene comprising the nucleic acid sequence SEQ ID NO. 4040, preferably the coding region thereof, conferred the production of or the increase in valine compared with the wild type control.

The nucleic acid sequence of At4g34160 from Arabidopsis thaliana, e.g. as shown in the respective line in column 5 of Table I, application no. 6, has been published in F. And the activity of the gene product thereof is the activity of cyclin D.

Accordingly, in one embodiment, the process of the present invention for producing isoleucine in a non-human organism, like a microorganism or a plant or a part thereof, comprises increasing or generating the activity of a gene product with the activity of a gene product conferring the activity of “cyclin D”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said At4g34160, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At4g34160, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At4g34160, or a functional equivalent or a homolog thereof as depicted in column 8 of Table II, application no. 6, preferably a homolog or functional equivalent as depicted in column 8 of Table II B, application no. 6, and being depicted in the same respective line as said At4g34160, and preferably the activity is increased non-targeted, whereby the respective line discloses in column 7 the fine chemical isoleucine.

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 24311, preferably the coding region thereof, conferred the production of or the increase in isoleucine compared with the wild type control.

The nucleic acid sequence of At4g34160 from Arabidopsis thaliana, e.g. as shown in the respective line in column 5 of Table I, application no. 6, has been published in F. And the activity of the gene product thereof is the activity of cyclin D .

Accordingly, in one embodiment, the process of the present invention for producing leucine in a non-human organism, like a microorganism or a plant or a part thereof, comprises increasing or generating the activity of a gene product with the activity of a gene product conferring the activity of “cyclin D”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said At4g34160, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At4g34160, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At4g34160, or a functional equivalent or a homolog thereof as depicted in column 8 of Table II, application no. 6, preferably a homolog or functional equivalent as depicted in column 8 of Table II B, application no. 6, and being depicted in the same respective line as said At4g34160, and preferably the activity is increased non-targeted, whereby the respective line discloses in column 7 the fine chemical leucine.

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 24311, preferably the coding region thereof, conferred the production of or the increase in leucine compared with the wild type control.

The nucleic acid sequence of At4g34160 from Arabidopsis thaliana, e.g. as shown in the respective line in column 5 of Table I, application no. 6, has been published in F. And the activity of the gene product thereof is the activity of cyclin D .

Accordingly, in one embodiment, the process of the present invention for producing valine in a non-human organism, like a microorganism or a plant or a part thereof, comprises increasing or generating the activity of a gene product with the activity of a gene product conferring the activity of “cyclin D”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At4g34160, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At4g34160, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At4g34160, or a functional equivalent or a homolog thereof as depicted in column 8 of Table II, application no. 6, preferably a homolog or functional equivalent as depicted in column 8 of Table II B, application no. 6, and being depicted in the same respective line as said At4g34160, and preferably the activity is increased non-targeted, whereby the respective line discloses in column 7 the fine chemical valine.

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 24311, preferably the coding region thereof, conferred the production of or the increase in valine compared with the wild type control.

The nucleic acid sequence of At4g35310 from Arabidopsis thaliana, e.g. as shown in the respective line in column 5 of Table I, application no. 6, has been published in F. And the activity of the gene product thereof is the activity of calcium-dependent protein kinase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said At4g35310, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At4g35310, or a functional equivalent or a homolog thereof as depicted in column 8 of Table II, application no. 6, preferably a homolog or functional equivalent as depicted in column 8 of Table II B, application no. 6, and being depicted in the same respective line as said At4g35310, and preferably the activity is increased non-targeted, whereby the respective line discloses in column 7 the fine chemical isoleucine.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in isoleucine compared with the wild type control.

The nucleic acid sequence of At4g35310 from Arabidopsis thaliana, e.g. as shown in the respective line in column 5 of Table I, application no. 6, has been published in F. And the activity of the gene product thereof is the activity of calcium-dependent protein kinase.

Accordingly, in one embodiment, the process of the present invention for producing leucine in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said At4g35310, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At4g35310, or a functional equivalent or a homolog thereof as depicted in column 8 of Table II, application no. 6, preferably a homolog or functional equivalent as depicted in column 8 of Table II B, application no. 6, and being depicted in the same respective line as said At4g35310, and preferably the activity is increased non-targeted, whereby the respective line discloses in column 7 the fine chemical leucine.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in leucine compared with the wild type control.

The nucleic acid sequence of At4g35310 from Arabidopsis thaliana, e.g. as shown in the respective line in column 5 of Table I, application no. 6, has been published in F. And the activity of the gene product thereof is the activity of calcium-dependent protein kinase.

Accordingly, in one embodiment, the process of the present invention for producing valine in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At4g35310, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At4g35310, or a functional equivalent or a homolog thereof as depicted in column 8 of Table II, application no. 6, preferably a homolog or functional equivalent as depicted in column 8 of Table II B, application no. 6, and being depicted in the same respective line as said At4g35310, and preferably the activity is increased non-targeted, whereby the respective line discloses in column 7 the fine chemical valine.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in valine compared with the wild type control.

The nucleic acid sequence of At5g02760 from Arabidopsis thaliana, e.g. as shown in the respective line in column 5 of Table I, application no. 6, has been published in F. And the activity of the gene product thereof is the activity of protein phosphatase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said At5g02760, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At5g02760, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At5g02760, or a functional equivalent or a homolog thereof as depicted in column 8 of Table II, application no. 6, preferably a homolog or functional equivalent as depicted in column 8 of Table II B, application no. 6, and being depicted in the same respective line as said At5g02760, and preferably the activity is increased non-targeted, whereby the respective line discloses in column 7 the fine chemical isoleucine.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 68987, preferably the coding region thereof, conferred the production of or the increase in isoleucine compared with the wild type control.

The nucleic acid sequence of At5g16650 from Arabidopsis thaliana, e.g. as shown in the respective line in column 5 of Table I, application no. 6, has been published in F. And the activity of the gene product thereof is the activity of At5g16650-protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “At5g16650-protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said At5g16650, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At5g16650, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At5g16650, or a functional equivalent or a homolog thereof as depicted in column 8 of Table II, application no. 6, preferably a homolog or functional equivalent as depicted in column 8 of Table II B, application no. 6, and being depicted in the same respective line as said At5g16650, and preferably the activity is increased non-targeted, whereby the respective line discloses in column 7 the fine chemical isoleucine.

Accordingly, in one embodiment, the molecule which activity is to be increased in the process of the invention is the gene product with an activity as a “At5g16650-protein”, preferably it is the molecule of section (a) or (b) of this paragraph.

In particular, it was observed that in plants, especially in Arabidopsis thaliana, increasing or generating the activity of a gene product non-targeted with the activity of a “At5g16650-protein”, preferably being encoded by a gene comprising the nucleic acid sequence SEQ ID NO. 25283, preferably the coding region thereof, conferred the production of or the increase in isoleucine compared with the wild type control.

The nucleic acid sequence of At5g16650 from Arabidopsis thaliana, e.g. as shown in the respective line in column 5 of Table I, application no. 6, has been published in F. And the activity of the gene product thereof is the activity of At5g16650-protein.

Accordingly, in one embodiment, the process of the present invention for producing valine in a non-human organism, like a microorganism or a plant or a part thereof, comprises increasing or generating the activity of a gene product with the activity of a gene product conferring the activity of “At5g16650-protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At5g16650, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At5g16650, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At5g16650, or a functional equivalent or a homolog thereof as depicted in column 8 of Table II, application no. 6, preferably a homolog or functional equivalent as depicted in column 8 of Table II B, application no. 6, and being depicted in the same respective line as said At5g16650, and preferably the activity is increased non-targeted, whereby the respective line discloses in column 7 the fine chemical valine.

Accordingly, in one embodiment, the molecule which activity is to be increased in the process of the invention is the gene product with an activity as a “At5g16650-protein”, preferably it is the molecule of section (a) or (b) of this paragraph.

In particular, it was observed that in plants, especially in Arabidopsis thaliana, increasing or generating the activity of a gene product non-targeted with the activity of a “At5g16650-protein”, preferably being encoded by a gene comprising the nucleic acid sequence SEQ ID NO. 25283, preferably the coding region thereof, conferred the production of or the increase in valine compared with the wild type control.

The nucleic acid sequence of At5g18600 from Arabidopsis thaliana, e.g. as shown in the respective line in column 5 of Table I, application no. 6, has been published in F. And the activity of the gene product thereof is the activity of monothiol glutaredoxin.

Accordingly, in one embodiment, the process of the present invention for producing valine in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At5g18600, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At5g18600, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At5g18600, or a functional equivalent or a homolog thereof as depicted in column 8 of Table II, application no. 6, preferably a homolog or functional equivalent as depicted in column 8 of Table II B, application no. 6, and being depicted in the same respective line as said At5g18600, and preferably the activity is increased non-targeted, whereby the respective line discloses in column 7 the fine chemical valine.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 4904, preferably the coding region thereof, conferred the production of or the increase in valine compared with the wild type control.

The nucleic acid sequence of At5g39950 from Arabidopsis thaliana, e.g. as shown in the respective line in column 5 of Table I, application no. 6, has been published in F. And the activity of the gene product thereof is the activity of thioredoxin.

Accordingly, in one embodiment, the process of the present invention for producing leucine in a non-human organism, like a microorganism or a plant or a part thereof, comprises increasing or generating the activity of a gene product with the activity of a gene product conferring the activity of “thioredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said At5g39950, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At5g39950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At5g39950, or a functional equivalent or a homolog thereof as depicted in column 8 of Table II, application no. 6, preferably a homolog or functional equivalent as depicted in column 8 of Table II B, application no. 6, and being depicted in the same respective line as said At5g39950, and preferably the activity is increased non-targeted, whereby the respective line discloses in column 7 the fine chemical leucine.

Accordingly, in one embodiment, the molecule which activity is to be increased in the process of the invention is the gene product with an activity as a “thioredoxin”, preferably it is the molecule of section (a) or (b) of this paragraph.

In particular, it was observed that in plants, especially in Arabidopsis thaliana, increasing or generating the activity of a gene product non-targeted with the activity of a “thioredoxin”, preferably being encoded by a gene comprising the nucleic acid sequence SEQ ID NO. 69574, preferably the coding region thereof, conferred the production of or the increase in leucine compared with the wild type control.

The nucleic acid sequence of At5g39950 from Arabidopsis thaliana, e.g. as shown in the respective line in column 5 of Table I, application no. 6, has been published in F. And the activity of the gene product thereof is the activity of thioredoxin.

Accordingly, in one embodiment, the process of the present invention for producing valine in a non-human organism, like a microorganism or a plant or a part thereof, comprises increasing or generating the activity of a gene product with the activity of a gene product conferring the activity of “thioredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At5g39950, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At5g39950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At5g39950, or a functional equivalent or a homolog thereof as depicted in column 8 of Table II, application no. 6, preferably a homolog or functional equivalent as depicted in column 8 of Table II B, application no. 6, and being depicted in the same respective line as said At5g39950, and preferably the activity is increased non-targeted, whereby the respective line discloses in column 7 the fine chemical valine.

Accordingly, in one embodiment, the molecule which activity is to be increased in the process of the invention is the gene product with an activity as a “thioredoxin”, preferably it is the molecule of section (a) or (b) of this paragraph.

In particular, it was observed that in plants, especially in Arabidopsis thaliana, increasing or generating the activity of a gene product non-targeted with the activity of a “thioredoxin”, preferably being encoded by a gene comprising the nucleic acid sequence SEQ ID NO. 69574, preferably the coding region thereof, conferred the production of or the increase in valine compared with the wild type control.

The nucleic acid sequence of At5g45060 from Arabidopsis thaliana, e.g. as shown in the respective line in column 5 of Table I, application no. 6, has been published in F. And the activity of the gene product thereof is the activity of disease resistance protein.

Accordingly, in one embodiment, the process of the present invention for producing valine in a non-human organism, like a microorganism or a plant or a part thereof, comprises increasing or generating the activity of a gene product with the activity of a gene product conferring the activity of “disease resistance protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At5g45060, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At5g45060, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At5g45060, or a functional equivalent or a homolog thereof as depicted in column 8 of Table II, application no. 6, preferably a homolog or functional equivalent as depicted in column 8 of Table II B, application no. 6, and being depicted in the same respective line as said At5g45060, and preferably the activity is increased non-targeted, whereby the respective line discloses in column 7 the fine chemical valine.

Accordingly, in one embodiment, the molecule which activity is to be increased in the process of the invention is the gene product with an activity as a “disease resistance protein”, preferably it is the molecule of section (a) or (b) of this paragraph.

In particular, it was observed that in plants, especially in Arabidopsis thaliana, increasing or generating the activity of a gene product non-targeted with the activity of a “disease resistance protein”, preferably being encoded by a gene comprising the nucleic acid sequence SEQ ID NO. 102223, preferably the coding region thereof, conferred the production of or the increase in valine compared with the wild type control.

The nucleic acid sequence of At5g56350 from Arabidopsis thaliana, e.g. as shown in the respective line in column 5 of Table I, application no. 6, has been published in F. And the activity of the gene product thereof is the activity of pyruvate kinase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “pyruvate kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said At5g56350, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At5g56350, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At5g56350, or a functional equivalent or a homolog thereof as depicted in column 8 of Table II, application no. 6, preferably a homolog or functional equivalent as depicted in column 8 of Table II B, application no. 6, and being depicted in the same respective line as said At5g56350, and preferably the activity is increased plastidic, whereby the respective line discloses in column 7 the fine chemical isoleucine.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 102373, preferably the coding region thereof, conferred the production of or the increase in isoleucine compared with the wild type control.

The nucleic acid sequence of At5g56350 from Arabidopsis thaliana, e.g. as shown in the respective line in column 5 of Table I, application no. 6, has been published in F. And the activity of the gene product thereof is the activity of pyruvate kinase.

Accordingly, in one embodiment, the process of the present invention for producing leucine in a non-human organism, like a microorganism or a plant or a part thereof, comprises increasing or generating the activity of a gene product with the activity of a gene product conferring the activity of “pyruvate kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said At5g56350, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At5g56350, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At5g56350, or a functional equivalent or a homolog thereof as depicted in column 8 of Table II, application no. 6, preferably a homolog or functional equivalent as depicted in column 8 of Table II B, application no. 6, and being depicted in the same respective line as said At5g56350, and preferably the activity is increased plastidic, whereby the respective line discloses in column 7 the fine chemical leucine.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 102373, preferably the coding region thereof, conferred the production of or the increase in leucine compared with the wild type control.

The nucleic acid sequence of At5g60110 from Arabidopsis thaliana, e.g. as shown in the respective line in column 5 of Table I, application no. 6, has been published in F. And the activity of the gene product thereof is the activity of RNA-binding protein.

Accordingly, in one embodiment, the process of the present invention for producing valine in a non-human organism, like a microorganism or a plant or a part thereof, comprises increasing or generating the activity of a gene product with the activity of a gene product conferring the activity of “RNA-binding protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At5g60110, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At5g60110, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At5g60110, or a functional equivalent or a homolog thereof as depicted in column 8 of Table II, application no. 6, preferably a homolog or functional equivalent as depicted in column 8 of Table II B, application no. 6, and being depicted in the same respective line as said At5g60110, and preferably the activity is increased non-targeted, whereby the respective line discloses in column 7 the fine chemical valine.

Accordingly, in one embodiment, the molecule which activity is to be increased in the process of the invention is the gene product with an activity as a “RNA-binding protein”, preferably it is the molecule of section (a) or (b) of this paragraph.

In particular, it was observed that in plants, especially in Arabidopsis thaliana, increasing or generating the activity of a gene product non-targeted with the activity of a “RNA-binding protein”, preferably being encoded by a gene comprising the nucleic acid sequence SEQ ID NO. 70006, preferably the coding region thereof, conferred the production of or the increase in valine compared with the wild type control.

The nucleic acid sequence of At5g60440 from Arabidopsis thaliana, e.g. as shown in the respective line in column 5 of Table I, application no. 6, has been published in F. And the activity of the gene product thereof is the activity of MADS-box transcription factor.

Accordingly, in one embodiment, the process of the present invention for producing isoleucine in a non-human organism, like a microorganism or a plant or a part thereof, comprises increasing or generating the activity of a gene product with the activity of a gene product conferring the activity of “MADS-box transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said At5g60440, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At5g60440, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At5g60440, or a functional equivalent or a homolog thereof as depicted in column 8 of Table II, application no. 6, preferably a homolog or functional equivalent as depicted in column 8 of Table II B, application no. 6, and being depicted in the same respective line as said At5g60440, and preferably the activity is increased non-targeted, whereby the respective line discloses in column 7 the fine chemical isoleucine.

Accordingly, in one embodiment, the molecule which activity is to be increased in the process of the invention is the gene product with an activity as a “MADS-box transcription factor”, preferably it is the molecule of section (a) or (b) of this paragraph.

In particular, it was observed that in plants, especially in Arabidopsis thaliana, increasing or generating the activity of a gene product non-targeted with the activity of a “MADS-box transcription factor”, preferably being encoded by a gene comprising the nucleic acid sequence SEQ ID NO. 102899, preferably the coding region thereof, conferred the production of or the increase in isoleucine compared with the wild type control.

The nucleic acid sequence of At5g60440 from Arabidopsis thaliana, e.g. as shown in the respective line in column 5 of Table I, application no. 6, has been published in F. And the activity of the gene product thereof is the activity of MADS-box transcription factor.

Accordingly, in one embodiment, the process of the present invention for producing leucine in a non-human organism, like a microorganism or a plant or a part thereof, comprises increasing or generating the activity of a gene product with the activity of a gene product conferring the activity of “MADS-box transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said At5g60440, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At5g60440, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At5g60440, or a functional equivalent or a homolog thereof as depicted in column 8 of Table II, application no. 6, preferably a homolog or functional equivalent as depicted in column 8 of Table II B, application no. 6, and being depicted in the same respective line as said At5g60440, and preferably the activity is increased non-targeted, whereby the respective line discloses in column 7 the fine chemical leucine.

Accordingly, in one embodiment, the molecule which activity is to be increased in the process of the invention is the gene product with an activity as a “MADS-box transcription factor”, preferably it is the molecule of section (a) or (b) of this paragraph.

In particular, it was observed that in plants, especially in Arabidopsis thaliana, increasing or generating the activity of a gene product non-targeted with the activity of a “MADS-box transcription factor”, preferably being encoded by a gene comprising the nucleic acid sequence SEQ ID NO. 102899, preferably the coding region thereof, conferred the production of or the increase in leucine compared with the wild type control.

The nucleic acid sequence of At5g63680 from Arabidopsis thaliana, e.g. as shown in the respective line in column 5 of Table I, application no. 6, has been published in F. And the activity of the gene product thereof is the activity of pyruvate kinase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “pyruvate kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said At5g63680, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At5g63680, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At5g63680, or a functional equivalent or a homolog thereof as depicted in column 8 of Table II, application no. 6, preferably a homolog or functional equivalent as depicted in column 8 of Table II B, application no. 6, and being depicted in the same respective line as said At5g63680, and preferably the activity is increased plastidic, whereby the respective line discloses in column 7 the fine chemical isoleucine.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 70038, preferably the coding region thereof, conferred the production of or the increase in isoleucine compared with the wild type control.

The nucleic acid sequence of At5g63680 from Arabidopsis thaliana, e.g. as shown in the respective line in column 5 of Table I, application no. 6, has been published in F. And the activity of the gene product thereof is the activity of pyruvate kinase.

Accordingly, in one embodiment, the process of the present invention for producing leucine in a non-human organism, like a microorganism or a plant or a part thereof, comprises increasing or generating the activity of a gene product with the activity of a gene product conferring the activity of “pyruvate kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said At5g63680, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At5g63680, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At5g63680, or a functional equivalent or a homolog thereof as depicted in column 8 of Table II, application no. 6, preferably a homolog or functional equivalent as depicted in column 8 of Table II B, application no. 6, and being depicted in the same respective line as said At5g63680, and preferably the activity is increased plastidic, whereby the respective line discloses in column 7 the fine chemical leucine.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 70038, preferably the coding region thereof, conferred the production of or the increase in leucine compared with the wild type control.

The nucleic acid sequence of At5g63680 from Arabidopsis thaliana, e.g. as shown in the respective line in column 5 of Table I, application no. 6, has been published in F. And the activity of the gene product thereof is the activity of pyruvate kinase.

Accordingly, in one embodiment, the process of the present invention for producing valine in a non-human organism, like a microorganism or a plant or a part thereof, comprises increasing or generating the activity of a gene product with the activity of a gene product conferring the activity of “pyruvate kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At5g63680, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At5g63680, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At5g63680, or a functional equivalent or a homolog thereof as depicted in column 8 of Table II, application no. 6, preferably a homolog or functional equivalent as depicted in column 8 of Table II B, application no. 6, and being depicted in the same respective line as said At5g63680, and preferably the activity is increased plastidic, whereby the respective line discloses in column 7 the fine chemical valine.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 70038, preferably the coding region thereof, conferred the production of or the increase in valine compared with the wild type control.

The nucleic acid sequence of At5g64920 from Arabidopsis thaliana, e.g. as shown in the respective line in column 5 of Table I, application no. 6, has been published in F. And the activity of the gene product thereof is the activity of zinc finger protein.

Accordingly, in one embodiment, the process of the present invention for producing valine in a non-human organism, like a microorganism or a plant or a part thereof, comprises increasing or generating the activity of a gene product with the activity of a gene product conferring the activity of “zinc finger protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said At5g64920, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said At5g64920, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said At5g64920, or a functional equivalent or a homolog thereof as depicted in column 8 of Table II, application no. 6, preferably a homolog or functional equivalent as depicted in column 8 of Table II B, application no. 6, and being depicted in the same respective line as said At5g64920, and preferably the activity is increased non-targeted, whereby the respective line discloses in column 7 the fine chemical valine.

Accordingly, in one embodiment, the molecule which activity is to be increased in the process of the invention is the gene product with an activity as a “zinc finger protein”, preferably it is the molecule of section (a) or (b) of this paragraph.

In particular, it was observed that in plants, especially in Arabidopsis thaliana, increasing or generating the activity of a gene product non-targeted with the activity of a “zinc finger protein”, preferably being encoded by a gene comprising the nucleic acid sequence SEQ ID NO. 5493, preferably the coding region thereof, conferred the production of or the increase in valine compared with the wild type control.

The nucleic acid sequence of AvinDRAFT_{—}1495 from Azotobacter vinelandii, e.g. as shown in the respective line in column 5 of Table I, application no. 6, has been published in D. And the activity of the gene product thereof is the activity of malic enzyme.

Accordingly, in one embodiment, the process of the present invention for producing isoleucine in a non-human organism, like a microorganism or a plant or a part thereof, comprises increasing or generating the activity of a gene product with the activity of a gene product conferring the activity of “malic enzyme”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said AvinDRAFT_{—}1495, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}1495, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}1495, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}1495, and preferably the activity is increased non-targeted, whereby the respective line discloses in column 7 the fine chemical isoleucine.

Accordingly, in one embodiment, the molecule which activity is to be increased in the process of the invention is the gene product with an activity as a “malic enzyme”, preferably it is the molecule of section (a) or (b) of this paragraph.

In particular, it was observed that in plants, especially in Arabidopsis thaliana, increasing or generating the activity of a gene product non-targeted with the activity of a “malic enzyme”, preferably being encoded by a gene comprising the nucleic acid sequence SEQ ID NO. 5557, preferably the coding region thereof, conferred the production of or the increase in isoleucine compared with the wild type control.

The nucleic acid sequence of AvinDRAFT_{—}1495 from Azotobacter vinelandii, e.g. as shown in the respective line in column 5 of Table I, application no. 6, has been published in D. And the activity of the gene product thereof is the activity of malic enzyme.

Accordingly, in one embodiment, the process of the present invention for producing leucine in a non-human organism, like a microorganism or a plant or a part thereof, comprises increasing or generating the activity of a gene product with the activity of a gene product conferring the activity of “malic enzyme”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said AvinDRAFT_{—}1495, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}1495, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}1495, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}1495, and preferably the activity is increased non-targeted, whereby the respective line discloses in column 7 the fine chemical leucine.

Accordingly, in one embodiment, the molecule which activity is to be increased in the process of the invention is the gene product with an activity as a “malic enzyme”, preferably it is the molecule of section (a) or (b) of this paragraph.

In particular, it was observed that in plants, especially in Arabidopsis thaliana, increasing or generating the activity of a gene product non-targeted with the activity of a “malic enzyme”, preferably being encoded by a gene comprising the nucleic acid sequence SEQ ID NO. 5557, preferably the coding region thereof, conferred the production of or the increase in leucine compared with the wild type control.

The nucleic acid sequence of AvinDRAFT_{—}1495 from Azotobacter vinelandii, e.g. as shown in the respective line in column 5 of Table I, application no. 6, has been published in D. And the activity of the gene product thereof is the activity of malic enzyme.

Accordingly, in one embodiment, the process of the present invention for producing valine in a non-human organism, like a microorganism or a plant or a part thereof, comprises increasing or generating the activity of a gene product with the activity of a gene product conferring the activity of “malic enzyme”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said AvinDRAFT_{—}1495, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}1495, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}1495, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}1495, and preferably the activity is increased non-targeted, whereby the respective line discloses in column 7 the fine chemical valine.

Accordingly, in one embodiment, the molecule which activity is to be increased in the process of the invention is the gene product with an activity as a “malic enzyme”, preferably it is the molecule of section (a) or (b) of this paragraph.

In particular, it was observed that in plants, especially in Arabidopsis thaliana, increasing or generating the activity of a gene product non-targeted with the activity of a “malic enzyme”, preferably being encoded by a gene comprising the nucleic acid sequence SEQ ID NO. 5557, preferably the coding region thereof, conferred the production of or the increase in valine compared with the wild type control.

The nucleic acid sequence of AvinDRAFT_{—}1806 from Azotobacter vinelandii, e.g. as shown in the respective line in column 5 of Table I, application no. 6, has been published in D. And the activity of the gene product thereof is the activity of pyruvate kinase.

Accordingly, in one embodiment, the process of the present invention for producing valine in a non-human organism, like a microorganism or a plant or a part thereof, comprises increasing or generating the activity of a gene product with the activity of a gene product conferring the activity of “pyruvate kinase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said AvinDRAFT_{—}1806, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}1806, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}1806, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}1806, and preferably the activity is increased non-targeted, whereby the respective line discloses in column 7 the fine chemical valine.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product non-targeted with the activity of a “pyruvate kinase”, preferably being encoded by a gene comprising the nucleic acid sequence SEQ ID NO. 26434, preferably the coding region thereof, conferred the production of or the increase in valine compared with the wild type control.

The nucleic acid sequence of AvinDRAFT_{—}2091 from Azotobacter vinelandii, e.g. as shown in the respective line in column 5 of Table I, application no. 6, has been published in D. And the activity of the gene product thereof is the activity of beta-hydroxylase.

Accordingly, in one embodiment, the process of the present invention for producing valine in a non-human organism, like a microorganism or a plant or a part thereof, comprises increasing or generating the activity of a gene product with the activity of a gene product conferring the activity of “beta-hydroxylase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said AvinDRAFT_{—}2091, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}2091, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}2091, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}2091, and preferably the activity is increased non-targeted, whereby the respective line discloses in column 7 the fine chemical valine.

Accordingly, in one embodiment, the molecule which activity is to be increased in the process of the invention is the gene product with an activity as a “beta-hydroxylase”, preferably it is the molecule of section (a) or (b) of this paragraph.

In particular, it was observed that in plants, especially in Arabidopsis thaliana, increasing or generating the activity of a gene product non-targeted with the activity of a “beta-hydroxylase”, preferably being encoded by a gene comprising the nucleic acid sequence SEQ ID NO. 6040, preferably the coding region thereof, conferred the production of or the increase in valine compared with the wild type control.

The nucleic acid sequence of AvinDRAFT_{—}2344 from Azotobacter vinelandii, e.g. as shown in the respective line in column 5 of Table I, application no. 6, has been published in D. And the activity of the gene product thereof is the activity of elongation factor Tu.

Accordingly, in one embodiment, the process of the present invention for producing isoleucine in a non-human organism, like a microorganism or a plant or a part thereof, comprises increasing or generating the activity of a gene product with the activity of a gene product conferring the activity of “elongation factor Tu”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said AvinDRAFT_{—}2344, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}2344, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAF_{—}2344, T or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}2344, and preferably the activity is increased non-targeted, whereby the respective line discloses in column 7 the fine chemical isoleucine.

Accordingly, in one embodiment, the molecule which activity is to be increased in the process of the invention is the gene product with an activity as a “elongation factor Tu”, preferably it is the molecule of section (a) or (b) of this paragraph.

In particular, it was observed that in plants, especially in Arabidopsis thaliana, increasing or generating the activity of a gene product non-targeted with the activity of a “elongation factor Tu”, preferably being encoded by a gene comprising the nucleic acid sequence SEQ ID NO. 27021, preferably the coding region thereof, conferred the production of or the increase in isoleucine compared with the wild type control.

The nucleic acid sequence of AvinDRAFT_{—}2344 from Azotobacter vinelandii, e.g. as shown in the respective line in column 5 of Table I, application no. 6, has been published in D. And the activity of the gene product thereof is the activity of elongation factor Tu.

Accordingly, in one embodiment, the process of the present invention for producing leucine in a non-human organism, like a microorganism or a plant or a part thereof, comprises increasing or generating the activity of a gene product with the activity of a gene product conferring the activity of “elongation factor Tu”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said AvinDRAFT_{—}2344, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}2344, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}2344, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}2344, and preferably the activity is increased non-targeted, whereby the respective line discloses in column 7 the fine chemical leucine.

Accordingly, in one embodiment, the molecule which activity is to be increased in the process of the invention is the gene product with an activity as a “elongation factor Tu”, preferably it is the molecule of section (a) or (b) of this paragraph.

In particular, it was observed that in plants, especially in Arabidopsis thaliana, increasing or generating the activity of a gene product non-targeted with the activity of a “elongation factor Tu”, preferably being encoded by a gene comprising the nucleic acid sequence SEQ ID NO. 27021, preferably the coding region thereof, conferred the production of or the increase in leucine compared with the wild type control.

The nucleic acid sequence of AvinDRAFT_{—}2344 from Azotobacter vinelandii, e.g. as shown in the respective line in column 5 of Table I, application no. 6, has been published in D. And the activity of the gene product thereof is the activity of elongation factor Tu.

Accordingly, in one embodiment, the process of the present invention for producing valine in a non-human organism, like a microorganism or a plant or a part thereof, comprises increasing or generating the activity of a gene product with the activity of a gene product conferring the activity of “elongation factor Tu”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said AvinDRAFT_{—}2344, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}2344, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}2344, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}2344, and preferably the activity is increased non-targeted, whereby the respective line discloses in column 7 the fine chemical valine.

Accordingly, in one embodiment, the molecule which activity is to be increased in the process of the invention is the gene product with an activity as a “elongation factor Tu”, preferably it is the molecule of section (a) or (b) of this paragraph.

In particular, it was observed that in plants, especially in Arabidopsis thaliana, increasing or generating the activity of a gene product non-targeted with the activity of a “elongation factor Tu”, preferably being encoded by a gene comprising the nucleic acid sequence SEQ ID NO. 27021, preferably the coding region thereof, conferred the production of or the increase in valine compared with the wild type control.

The nucleic acid sequence of AvinDRAFT_{—}2369 from Azotobacter vinelandii, e.g. as shown in the respective line in column 5 of Table I, application no. 6, has been published in D. And the activity of the gene product thereof is the activity of 50S ribosomal protein L14.

Accordingly, in one embodiment, the process of the present invention for producing valine in a non-human organism, like a microorganism or a plant or a part thereof, comprises increasing or generating the activity of a gene product with the activity of a gene product conferring the activity of “505 ribosomal protein L14”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said AvinDRAFT_{—}2369, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}2369, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}2369, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}2369, and preferably the activity is increased non-targeted, whereby the respective line discloses in column 7 the fine chemical valine.

Accordingly, in one embodiment, the molecule which activity is to be increased in the process of the invention is the gene product with an activity as a “505 ribosomal protein L14”, preferably it is the molecule of section (a) or (b) of this paragraph.

In particular, it was observed that in plants, especially in Arabidopsis thaliana, increasing or generating the activity of a gene product non-targeted with the activity of a “505 ribosomal protein L14”, preferably being encoded by a gene comprising the nucleic acid sequence SEQ ID NO. 71763, preferably the coding region thereof, conferred the production of or the increase in valine compared with the wild type control.

The nucleic acid sequence of AvinDRAFT_{—}2521 from Azotobacter vinelandii, e.g. as shown in the respective line in column 5 of Table I, application no. 6, has been published in D. And the activity of the gene product thereof is the activity of ABC transporter permease protein.

Accordingly, in one embodiment, the process of the present invention for producing valine in a non-human organism, like a microorganism or a plant or a part thereof, comprises increasing or generating the activity of a gene product with the activity of a gene product conferring the activity of “ABC transporter permease protein”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said AvinDRAFT_{—}2521, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}2521, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}2521, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}2521, and preferably the activity is increased non-targeted, whereby the respective line discloses in column 7 the fine chemical valine.

Accordingly, in one embodiment, the molecule which activity is to be increased in the process of the invention is the gene product with an activity as a “ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “ABC transporter permease protein”, preferably being encoded by a gene comprising the nucleic acid sequence SEQ ID NO. 27882, preferably the coding region thereof, conferred the production of or the increase in valine compared with the wild type control.

The nucleic acid sequence of AvinDRAFT_{—}3159 from Azotobacter vinelandii, e.g. as shown in the respective line in column 5 of Table I, application no. 6, has been published in D. And the activity of the gene product thereof is the activity of enoyl-CoA hydratase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “enoyl-CoA hydratase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said AvinDRAFT_{—}3159, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}3159, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}3159, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}3159, and preferably the activity is increased non-targeted, whereby the respective line discloses in column 7 the fine chemical isoleucine.

Accordingly, in one embodiment, the molecule which activity is to be increased in the process of the invention is the gene product with an activity as a “enoyl-CoA hydratase”, preferably it is the molecule of section (a) or (b) of this paragraph.

In particular, it was observed that in plants, especially in Arabidopsis thaliana, increasing or generating the activity of a gene product non-targeted with the activity of a “enoyl-CoA hydratase”, preferably being encoded by a gene comprising the nucleic acid sequence SEQ ID NO. 28738, preferably the coding region thereof, conferred the production of or the increase in isoleucine compared with the wild type control.

The nucleic acid sequence of AvinDRAFT_{—}3159 from Azotobacter vinelandii, e.g. as shown in the respective line in column 5 of Table I, application no. 6, has been published in D. And the activity of the gene product thereof is the activity of enoyl-CoA hydratase.

Accordingly, in one embodiment, the process of the present invention for producing leucine in a non-human organism, like a microorganism or a plant or a part thereof, comprises increasing or generating the activity of a gene product with the activity of a gene product conferring the activity of “enoyl-CoA hydratase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said AvinDRAFT_{—}3159, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}3159, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}3159, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}3159, and preferably the activity is increased non-targeted, whereby the respective line discloses in column 7 the fine chemical leucine.

Accordingly, in one embodiment, the molecule which activity is to be increased in the process of the invention is the gene product with an activity as a “enoyl-CoA hydratase”, preferably it is the molecule of section (a) or (b) of this paragraph.

In particular, it was observed that in plants, especially in Arabidopsis thaliana, increasing or generating the activity of a gene product non-targeted with the activity of a “enoyl-CoA hydratase”, preferably being encoded by a gene comprising the nucleic acid sequence SEQ ID NO. 28738, preferably the coding region thereof, conferred the production of or the increase in leucine compared with the wild type control.

The nucleic acid sequence of AvinDRAFT_{—}3159 from Azotobacter vinelandii, e.g. as shown in the respective line in column 5 of Table I, application no. 6, has been published in D. And the activity of the gene product thereof is the activity of enoyl-CoA hydratase.

Accordingly, in one embodiment, the process of the present invention for producing valine in a non-human organism, like a microorganism or a plant or a part thereof, comprises increasing or generating the activity of a gene product with the activity of a gene product conferring the activity of “enoyl-CoA hydratase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said AvinDRAFT_{—}3159, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}3159, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}3159, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}3159, and preferably the activity is increased non-targeted, whereby the respective line discloses in column 7 the fine chemical valine.

Accordingly, in one embodiment, the molecule which activity is to be increased in the process of the invention is the gene product with an activity as a “enoyl-CoA hydratase”, preferably it is the molecule of section (a) or (b) of this paragraph.

In particular, it was observed that in plants, especially in Arabidopsis thaliana, increasing or generating the activity of a gene product non-targeted with the activity of a “enoyl-CoA hydratase”, preferably being encoded by a gene comprising the nucleic acid sequence SEQ ID NO. 28738, preferably the coding region thereof, conferred the production of or the increase in valine compared with the wild type control.

The nucleic acid sequence of AvinDRAFT_{—}3209 from Azotobacter vinelandii, e.g. as shown in the respective line in column 5 of Table I, application no. 6, has been published in D. And the activity of the gene product thereof is the activity of transcriptional regulator.

Accordingly, in one embodiment, the process of the present invention for producing isoleucine in a non-human organism, like a microorganism or a plant or a part thereof, comprises increasing or generating the activity of a gene product with the activity of a gene product conferring the activity of “transcriptional regulator”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said AvinDRAFT_{—}3209, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}3209, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}3209, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}3209, and preferably the activity is increased non-targeted, whereby the respective line discloses in column 7 the fine chemical isoleucine.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 29286, preferably the coding region thereof, conferred the production of or the increase in isoleucine compared with the wild type control.

The nucleic acid sequence of AvinDRAFT_{—}3209 from Azotobacter vinelandii, e.g. as shown in the respective line in column 5 of Table I, application no. 6, has been published in D. And the activity of the gene product thereof is the activity of transcriptional regulator.

Accordingly, in one embodiment, the process of the present invention for producing leucine in a non-human organism, like a microorganism or a plant or a part thereof, comprises increasing or generating the activity of a gene product with the activity of a gene product conferring the activity of “transcriptional regulator”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said AvinDRAFT_{—}3209, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}3209, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}3209, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}3209, and preferably the activity is increased non-targeted, whereby the respective line discloses in column 7 the fine chemical leucine.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 29286, preferably the coding region thereof, conferred the production of or the increase in leucine compared with the wild type control.

The nucleic acid sequence of AvinDRAFT_{—}3209 from Azotobacter vinelandii, e.g. as shown in the respective line in column 5 of Table I, application no. 6, has been published in D. And the activity of the gene product thereof is the activity of transcriptional regulator.

Accordingly, in one embodiment, the process of the present invention for producing valine in a non-human organism, like a microorganism or a plant or a part thereof, comprises increasing or generating the activity of a gene product with the activity of a gene product conferring the activity of “transcriptional regulator”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said AvinDRAFT_{—}3209, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}3209, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}3209, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}3209, and preferably the activity is increased non-targeted, whereby the respective line discloses in column 7 the fine chemical valine.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 29286, preferably the coding region thereof, conferred the production of or the increase in valine compared with the wild type control.

The nucleic acid sequence of AvinDRAFT_{—}3250 from Azotobacter vinelandii, e.g. as shown in the respective line in column 5 of Table I, application no. 6, has been published in D. And the activity of the gene product thereof is the activity of acyltransferase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “acyltransferase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said AvinDRAFT_{—}3250, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}3250, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}3250, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}3250, and preferably the activity is increased non-targeted, whereby the respective line discloses in column 7 the fine chemical isoleucine.

Accordingly, in one embodiment, the molecule which activity is to be increased in the process of the invention is the gene product with an activity as a “acyltransferase”, preferably it is the molecule of section (a) or (b) of this paragraph.

In particular, it was observed that in plants, especially in Arabidopsis thaliana, increasing or generating the activity of a gene product non-targeted with the activity of a “acyltransferase”, preferably being encoded by a gene comprising the nucleic acid sequence SEQ ID NO. 29397, preferably the coding region thereof, conferred the production of or the increase in isoleucine compared with the wild type control.

The nucleic acid sequence of AvinDRAFT_{—}3250 from Azotobacter vinelandii, e.g. as shown in the respective line in column 5 of Table I, application no. 6, has been published in D. And the activity of the gene product thereof is the activity of acyltransferase.

Accordingly, in one embodiment, the process of the present invention for producing leucine in a non-human organism, like a microorganism or a plant or a part thereof, comprises increasing or generating the activity of a gene product with the activity of a gene product conferring the activity of “acyltransferase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said AvinDRAFT_{—}3250, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}3250, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}3250, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}3250, and preferably the activity is increased non-targeted, whereby the respective line discloses in column 7 the fine chemical leucine.

Accordingly, in one embodiment, the molecule which activity is to be increased in the process of the invention is the gene product with an activity as a “acyltransferase”, preferably it is the molecule of section (a) or (b) of this paragraph.

In particular, it was observed that in plants, especially in Arabidopsis thaliana, increasing or generating the activity of a gene product non-targeted with the activity of a “acyltransferase”, preferably being encoded by a gene comprising the nucleic acid sequence SEQ ID NO. 29397, preferably the coding region thereof, conferred the production of or the increase in leucine compared with the wild type control.

The nucleic acid sequence of AvinDRAFT_{—}3250 from Azotobacter vinelandii, e.g. as shown in the respective line in column 5 of Table I, application no. 6, has been published in D. And the activity of the gene product thereof is the activity of acyltransferase.

Accordingly, in one embodiment, the process of the present invention for producing valine in a non-human organism, like a microorganism or a plant or a part thereof, comprises increasing or generating the activity of a gene product with the activity of a gene product conferring the activity of “acyltransferase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said AvinDRAFT_{—}3250, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}3250, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}3250, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}3250, and preferably the activity is increased non-targeted, whereby the respective line discloses in column 7 the fine chemical valine.

Accordingly, in one embodiment, the molecule which activity is to be increased in the process of the invention is the gene product with an activity as a “acyltransferase”, preferably it is the molecule of section (a) or (b) of this paragraph.

In particular, it was observed that in plants, especially in Arabidopsis thaliana, increasing or generating the activity of a gene product non-targeted with the activity of a “acyltransferase”, preferably being encoded by a gene comprising the nucleic acid sequence SEQ ID NO. 29397, preferably the coding region thereof, conferred the production of or the increase in valine compared with the wild type control.

The nucleic acid sequence of AvinDRAFT_{—}3253 from Azotobacter vinelandii, e.g. as shown in the respective line in column 5 of Table I, application no. 6, has been published in D. And the activity of the gene product thereof is the activity of oxidoreductase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “oxidoreductase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said AvinDRAFT_{—}3253, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}3253, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}3253, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}3253, and preferably the activity is increased non-targeted, whereby the respective line discloses in column 7 the fine chemical isoleucine.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 29500, preferably the coding region thereof, conferred the production of or the increase in isoleucine compared with the wild type control.

The nucleic acid sequence of AvinDRAFT_{—}3253 from Azotobacter vinelandii, e.g. as shown in the respective line in column 5 of Table I, application no. 6, has been published in D. And the activity of the gene product thereof is the activity of oxidoreductase.

Accordingly, in one embodiment, the process of the present invention for producing leucine in a non-human organism, like a microorganism or a plant or a part thereof, comprises increasing or generating the activity of a gene product with the activity of a gene product conferring the activity of “oxidoreductase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said AvinDRAFT_{—}3253, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}3253, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}3253, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}3253, and preferably the activity is increased non-targeted, whereby the respective line discloses in column 7 the fine chemical leucine.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 29500, preferably the coding region thereof, conferred the production of or the increase in leucine compared with the wild type control.

The nucleic acid sequence of AvinDRAFT_{—}3253 from Azotobacter vinelandii, e.g. as shown in the respective line in column 5 of Table I, application no. 6, has been published in D. And the activity of the gene product thereof is the activity of oxidoreductase.

Accordingly, in one embodiment, the process of the present invention for producing valine in a non-human organism, like a microorganism or a plant or a part thereof, comprises increasing or generating the activity of a gene product with the activity of a gene product conferring the activity of “oxidoreductase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said AvinDRAFT_{—}3253, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}3253, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}3253, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}3253, and preferably the activity is increased non-targeted, whereby the respective line discloses in column 7 the fine chemical valine.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 29500, preferably the coding region thereof, conferred the production of or the increase in valine compared with the wild type control.

The nucleic acid sequence of AVINDRAFT_{—}4128 from Azotobacter vinelandii, e.g. as shown in the respective line in column 5 of Table I, application no. 6, has been published in D. And the activity of the gene product thereof is the activity of ABC transporter component.

Accordingly, in one embodiment, the process of the present invention for producing valine in a non-human organism, like a microorganism or a plant or a part thereof, comprises increasing or generating the activity of a gene product with the activity of a gene product conferring the activity of “ABC transporter component”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said AVINDRAFT_{—}4128, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AVINDRAFT_{—}4128, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AVINDRAFT_{—}4128, or a functional equivalent or a homolog thereof as depicted in column 8 of Table II, application no. 6, preferably a homolog or functional equivalent as depicted in column 8 of Table II B, application no. 6, and being depicted in the same respective line as said AVINDRAFT_{—}4128, and preferably the activity is increased non-targeted, whereby the respective line discloses in column 7 the fine chemical valine.

Accordingly, in one embodiment, the molecule which activity is to be increased in the process of the invention is the gene product with an activity as a “ABC transporter component”, preferably it is the molecule of section (a) or (b) of this paragraph.

In particular, it was observed that in plants, especially in Arabidopsis thaliana, increasing or generating the activity of a gene product non-targeted with the activity of a “ABC transporter component”, preferably being encoded by a gene comprising the nucleic acid sequence SEQ ID NO. 72520, preferably the coding region thereof, conferred the production of or the increase in valine compared with the wild type control.

The nucleic acid sequence of AvinDRAFT_{—}4384 from Azotobacter vinelandii, e.g. as shown in the respective line in column 5 of Table I, application no. 6, has been published in D. And the activity of the gene product thereof is the activity of glucose-1-phosphate cytidylyltransferase.

Accordingly, in one embodiment, the process of the present invention for producing valine in a non-human organism, like a microorganism or a plant or a part thereof, comprises increasing or generating the activity of a gene product with the activity of a gene product conferring the activity of “glucose-1-phosphate cytidylyltransferase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said AvinDRAFT_{—}4384, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}4384, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}4384, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}4384, and preferably the activity is increased non-targeted, whereby the respective line discloses in column 7 the fine chemical valine.

Accordingly, in one embodiment, the molecule which activity is to be increased in the process of the invention is the gene product with an activity as a “glucose-1-phosphate cytidylyltransferase”, preferably it is the molecule of section (a) or (b) of this paragraph.

In particular, it was observed that in plants, especially in Arabidopsis thaliana, increasing or generating the activity of a gene product non-targeted with the activity of a “glucose-1-phosphate cytidylyltransferase”, preferably being encoded by a gene comprising the nucleic acid sequence SEQ ID NO. 31717, preferably the coding region thereof, conferred the production of or the increase in valine compared with the wild type control.

The nucleic acid sequence of AvinDRAFT_{—}4562 from Azotobacter vinelandii, e.g. as shown in the respective line in column 5 of Table I, application no. 6, has been published in D. And the activity of the gene product thereof is the activity of aminotransferase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “aminotransferase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said AvinDRAFT_{—}4562, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}4562, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}4562, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}4562, and preferably the activity is increased non-targeted, whereby the respective line discloses in column 7 the fine chemical isoleucine.

Accordingly, in one embodiment, the molecule which activity is to be increased in the process of the invention is the gene product with an activity as a “aminotransferase”, preferably it is the molecule of section (a) or (b) of this paragraph.

In particular, it was observed that in plants, especially in Arabidopsis thaliana, increasing or generating the activity of a gene product non-targeted with the activity of a “aminotransferase”, preferably being encoded by a gene comprising the nucleic acid sequence SEQ ID NO. 31926, preferably the coding region thereof, conferred the production of or the increase in isoleucine compared with the wild type control.

The nucleic acid sequence of AvinDRAFT_{—}4562 from Azotobacter vinelandii, e.g. as shown in the respective line in column 5 of Table I, application no. 6, has been published in D. And the activity of the gene product thereof is the activity of aminotransferase.

Accordingly, in one embodiment, the process of the present invention for producing valine in a non-human organism, like a microorganism or a plant or a part thereof, comprises increasing or generating the activity of a gene product with the activity of a gene product conferring the activity of “aminotransferase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said AvinDRAFT_{—}4562, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}4562, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT _{—}4562, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}4562, and preferably the activity is increased non-targeted, whereby the respective line discloses in column 7 the fine chemical valine.

Accordingly, in one embodiment, the molecule which activity is to be increased in the process of the invention is the gene product with an activity as a “aminotransferase”, preferably it is the molecule of section (a) or (b) of this paragraph.

In particular, it was observed that in plants, especially in Arabidopsis thaliana, increasing or generating the activity of a gene product non-targeted with the activity of a “aminotransferase”, preferably being encoded by a gene comprising the nucleic acid sequence SEQ ID NO. 31926, preferably the coding region thereof, conferred the production of or the increase in valine compared with the wild type control.

The nucleic acid sequence of AvinDRAFT_{—}4606 from Azotobacter vinelandii, e.g. as shown in the respective line in column 5 of Table I, application no. 6, has been published in D. And the activity of the gene product thereof is the activity of NADH-quinone oxidoreductase subunit.

Accordingly, in one embodiment, the process of the present invention for producing isoleucine in a non-human organism, like a microorganism or a plant or a part thereof, comprises increasing or generating the activity of a gene product with the activity of a gene product conferring the activity of “NADH-quinone oxidoreductase subunit”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said AvinDRAFT_{—}4606, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}4606, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}4606, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}4606, and preferably the activity is increased non-targeted, whereby the respective line discloses in column 7 the fine chemical isoleucine.

Accordingly, in one embodiment, the molecule which activity is to be increased in the process of the invention is the gene product with an activity as a “NADH-quinone oxidoreductase subunit”, preferably it is the molecule of section (a) or (b) of this paragraph.

In particular, it was observed that in plants, especially in Arabidopsis thaliana, increasing or generating the activity of a gene product non-targeted with the activity of a “NADH-quinone oxidoreductase subunit”, preferably being encoded by a gene comprising the nucleic acid sequence SEQ ID NO. 73719, preferably the coding region thereof, conferred the production of or the increase in isoleucine compared with the wild type control.

The nucleic acid sequence of AvinDRAFT_{—}4606 from Azotobacter vinelandii, e.g. as shown in the respective line in column 5 of Table I, application no. 6, has been published in D. And the activity of the gene product thereof is the activity of NADH-quinone oxidoreductase subunit.

Accordingly, in one embodiment, the process of the present invention for producing leucine in a non-human organism, like a microorganism or a plant or a part thereof, comprises increasing or generating the activity of a gene product with the activity of a gene product conferring the activity of “NADH-quinone oxidoreductase subunit”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said AvinDRAFT_{—}4606, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}4606, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}4606, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}4606, and preferably the activity is increased non-targeted, whereby the respective line discloses in column 7 the fine chemical leucine.

Accordingly, in one embodiment, the molecule which activity is to be increased in the process of the invention is the gene product with an activity as a “NADH-quinone oxidoreductase subunit”, preferably it is the molecule of section (a) or (b) of this paragraph.

In particular, it was observed that in plants, especially in Arabidopsis thaliana, increasing or generating the activity of a gene product non-targeted with the activity of a “NADH-quinone oxidoreductase subunit”, preferably being encoded by a gene comprising the nucleic acid sequence SEQ ID NO. 73719, preferably the coding region thereof, conferred the production of or the increase in leucine compared with the wild type control.

The nucleic acid sequence of AvinDRAFT_{—}4836 from Azotobacter vinelandii, e.g. as shown in the respective line in column 5 of Table I, application no. 6, has been published in D. And the activity of the gene product thereof is the activity of glutamate-ammonia-ligase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “glutamate-ammonia-ligase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said AvinDRAFT_{—}4836, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}4836, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}4836, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}4836, and preferably the activity is increased non-targeted, whereby the respective line discloses in column 7 the fine chemical isoleucine.

Accordingly, in one embodiment, the molecule which activity is to be increased in the process of the invention is the gene product with an activity as a “glutamate-ammonia-ligase”, preferably it is the molecule of section (a) or (b) of this paragraph.

In particular, it was observed that in plants, especially in Arabidopsis thaliana, increasing or generating the activity of a gene product non-targeted with the activity of a “glutamate-ammonia-ligase”, preferably being encoded by a gene comprising the nucleic acid sequence SEQ ID NO. 74163, preferably the coding region thereof, conferred the production of or the increase in isoleucine compared with the wild type control.

The nucleic acid sequence of AvinDRAFT_{—}4836 from Azotobacter vinelandii, e.g. as shown in the respective line in column 5 of Table I, application no. 6, has been published in D. And the activity of the gene product thereof is the activity of glutamate-ammonia-ligase.

Accordingly, in one embodiment, the process of the present invention for producing leucine in a non-human organism, like a microorganism or a plant or a part thereof, comprises increasing or generating the activity of a gene product with the activity of a gene product conferring the activity of “glutamate-ammonia-ligase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said AvinDRAFT_{—}4836, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}4836, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}4836, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}4836, and preferably the activity is increased non-targeted, whereby the respective line discloses in column 7 the fine chemical leucine.

Accordingly, in one embodiment, the molecule which activity is to be increased in the process of the invention is the gene product with an activity as a “glutamate-ammonia-ligase”, preferably it is the molecule of section (a) or (b) of this paragraph.

In particular, it was observed that in plants, especially in Arabidopsis thaliana, increasing or generating the activity of a gene product non-targeted with the activity of a “glutamate-ammonia-ligase”, preferably being encoded by a gene comprising the nucleic acid sequence SEQ ID NO. 74163, preferably the coding region thereof, conferred the production of or the increase in leucine compared with the wild type control.

The nucleic acid sequence of AvinDRAFT_{—}4836 from Azotobacter vinelandii, e.g. as shown in the respective line in column 5 of Table I, application no. 6, has been published in D. And the activity of the gene product thereof is the activity of glutamate-ammonia-ligase.

Accordingly, in one embodiment, the process of the present invention for producing valine in a non-human organism, like a microorganism or a plant or a part thereof, comprises increasing or generating the activity of a gene product with the activity of a gene product conferring the activity of “glutamate-ammonia-ligase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said AvinDRAFT_{—}4836, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}4836, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}4836, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}4836, and preferably the activity is increased non-targeted, whereby the respective line discloses in column 7 the fine chemical valine.

Accordingly, in one embodiment, the molecule which activity is to be increased in the process of the invention is the gene product with an activity as a “glutamate-ammonia-ligase”, preferably it is the molecule of section (a) or (b) of this paragraph.

In particular, it was observed that in plants, especially in Arabidopsis thaliana, increasing or generating the activity of a gene product non-targeted with the activity of a “glutamate-ammonia-ligase”, preferably being encoded by a gene comprising the nucleic acid sequence SEQ ID NO. 74163, preferably the coding region thereof, conferred the production of or the increase in valine compared with the wild type control.

The nucleic acid sequence of AvinDRAFT_{—}4847 from Azotobacter vinelandii, e.g. as shown in the respective line in column 5 of Table I, application no. 6, has been published in D. And the activity of the gene product thereof is the activity of isopropylmalate isomerase large subunit.

Accordingly, in one embodiment, the process of the present invention for producing valine in a non-human organism, like a microorganism or a plant or a part thereof, comprises increasing or generating the activity of a gene product with the activity of a gene product conferring the activity of “isopropylmalate isomerase large subunit”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said AvinDRAFT_{—}4847, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}4847, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}4847, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}4847, and preferably the activity is increased non-targeted, whereby the respective line discloses in column 7 the fine chemical valine.

Accordingly, in one embodiment, the molecule which activity is to be increased in the process of the invention is the gene product with an activity as a “isopropylmalate isomerase large subunit”, preferably it is the molecule of section (a) or (b) of this paragraph.

In particular, it was observed that in plants, especially in Arabidopsis thaliana, increasing or generating the activity of a gene product non-targeted with the activity of a “isopropylmalate isomerase large subunit”, preferably being encoded by a gene comprising the nucleic acid sequence SEQ ID NO. 102941, preferably the coding region thereof, conferred the production of or the increase in valine compared with the wild type control.

The nucleic acid sequence of AvinDRAFT_{—}5103 from Azotobacter vinelandii, e.g. as shown in the respective line in column 5 of Table I, application no. 6, has been published in D. And the activity of the gene product thereof is the activity of hydrolase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “hydrolase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said AvinDRAFT_{—}5103, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}5103, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAF_{—}5103, T or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}5103, and preferably the activity is increased non-targeted, whereby the respective line discloses in column 7 the fine chemical isoleucine.

Accordingly, in one embodiment, the molecule which activity is to be increased in the process of the invention is the gene product with an activity as a “hydrolase”, preferably it is the molecule of section (a) or (b) of this paragraph.

In particular, it was observed that in plants, especially in Arabidopsis thaliana, increasing or generating the activity of a gene product non-targeted with the activity of a “hydrolase”, preferably being encoded by a gene comprising the nucleic acid sequence SEQ ID NO. 6510, preferably the coding region thereof, conferred the production of or the increase in isoleucine compared with the wild type control.

The nucleic acid sequence of AvinDRAFT_{—}5103 from Azotobacter vinelandii, e.g. as shown in the respective line in column 5 of Table I, application no. 6, has been published in D. And the activity of the gene product thereof is the activity of hydrolase.

Accordingly, in one embodiment, the process of the present invention for producing leucine in a non-human organism, like a microorganism or a plant or a part thereof, comprises increasing or generating the activity of a gene product with the activity of a gene product conferring the activity of “hydrolase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said AvinDRAFT_{—}5103, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}5103, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAF_{—}5103, T or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}5103, and preferably the activity is increased non-targeted, whereby the respective line discloses in column 7 the fine chemical leucine.

Accordingly, in one embodiment, the molecule which activity is to be increased in the process of the invention is the gene product with an activity as a “hydrolase”, preferably it is the molecule of section (a) or (b) of this paragraph.

In particular, it was observed that in plants, especially in Arabidopsis thaliana, increasing or generating the activity of a gene product non-targeted with the activity of a “hydrolase”, preferably being encoded by a gene comprising the nucleic acid sequence SEQ ID NO. 6510, preferably the coding region thereof, conferred the production of or the increase in leucine compared with the wild type control.

The nucleic acid sequence of AvinDRAFT_{—}5103 from Azotobacter vinelandii, e.g. as shown in the respective line in column 5 of Table I, application no. 6, has been published in D. And the activity of the gene product thereof is the activity of hydrolase.

Accordingly, in one embodiment, the process of the present invention for producing valine in a non-human organism, like a microorganism or a plant or a part thereof, comprises increasing or generating the activity of a gene product with the activity of a gene product conferring the activity of “hydrolase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said AvinDRAFT_{—}5103, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}5103, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}5103, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}5103, and preferably the activity is increased non-targeted, whereby the respective line discloses in column 7 the fine chemical valine.

Accordingly, in one embodiment, the molecule which activity is to be increased in the process of the invention is the gene product with an activity as a “hydrolase”, preferably it is the molecule of section (a) or (b) of this paragraph.

In particular, it was observed that in plants, especially in Arabidopsis thaliana, increasing or generating the activity of a gene product non-targeted with the activity of a “hydrolase”, preferably being encoded by a gene comprising the nucleic acid sequence SEQ ID NO. 6510, preferably the coding region thereof, conferred the production of or the increase in valine compared with the wild type control.

The nucleic acid sequence of AvinDRAFT_{—}5292 from Azotobacter vinelandii, e.g. as shown in the respective line in column 5 of Table I, application no. 6, has been published in D. And the activity of the gene product thereof is the activity of fumarylacetoacetate hydrolase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “fumarylacetoacetate hydrolase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said AvinDRAFT_{—}5292, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}5292, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}5292, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}5292, and preferably the activity is increased non-targeted, whereby the respective line discloses in column 7 the fine chemical isoleucine.

Accordingly, in one embodiment, the molecule which activity is to be increased in the process of the invention is the gene product with an activity as a “fumarylacetoacetate hydrolase”, preferably it is the molecule of section (a) or (b) of this paragraph.

In particular, it was observed that in plants, especially in Arabidopsis thaliana, increasing or generating the activity of a gene product non-targeted with the activity of a “fumarylacetoacetate hydrolase”, preferably being encoded by a gene comprising the nucleic acid sequence SEQ ID NO. 32308, preferably the coding region thereof, conferred the production of or the increase in isoleucine compared with the wild type control.

The nucleic acid sequence of AvinDRAFT_{—}5292 from Azotobacter vinelandii, e.g. as shown in the respective line in column 5 of Table I, application no. 6, has been published in D. And the activity of the gene product thereof is the activity of fumarylacetoacetate hydrolase.

Accordingly, in one embodiment, the process of the present invention for producing leucine in a non-human organism, like a microorganism or a plant or a part thereof, comprises increasing or generating the activity of a gene product with the activity of a gene product conferring the activity of “fumarylacetoacetate hydrolase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said AvinDRAFT_{—}5292, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}5292, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}5292, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}5292, and preferably the activity is increased non-targeted, whereby the respective line discloses in column 7 the fine chemical leucine.

Accordingly, in one embodiment, the molecule which activity is to be increased in the process of the invention is the gene product with an activity as a “fumarylacetoacetate hydrolase”, preferably it is the molecule of section (a) or (b) of this paragraph.

In particular, it was observed that in plants, especially in Arabidopsis thaliana, increasing or generating the activity of a gene product non-targeted with the activity of a “fumarylacetoacetate hydrolase”, preferably being encoded by a gene comprising the nucleic acid sequence SEQ ID NO. 32308, preferably the coding region thereof, conferred the production of or the increase in leucine compared with the wild type control.

The nucleic acid sequence of AvinDRAFT_{—}5292 from Azotobacter vinelandii, e.g. as shown in the respective line in column 5 of Table I, application no. 6, has been published in D. And the activity of the gene product thereof is the activity of fumarylacetoacetate hydrolase.

Accordingly, in one embodiment, the process of the present invention for producing valine in a non-human organism, like a microorganism or a plant or a part thereof, comprises increasing or generating the activity of a gene product with the activity of a gene product conferring the activity of “fumarylacetoacetate hydrolase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said AvinDRAFT_{—}5292, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}5292, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}5292, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}5292, and preferably the activity is increased non-targeted, whereby the respective line discloses in column 7 the fine chemical valine.

Accordingly, in one embodiment, the molecule which activity is to be increased in the process of the invention is the gene product with an activity as a “fumarylacetoacetate hydrolase”, preferably it is the molecule of section (a) or (b) of this paragraph.

In particular, it was observed that in plants, especially in Arabidopsis thaliana, increasing or generating the activity of a gene product non-targeted with the activity of a “fumarylacetoacetate hydrolase”, preferably being encoded by a gene comprising the nucleic acid sequence SEQ ID NO. 32308, preferably the coding region thereof, conferred the production of or the increase in valine compared with the wild type control.

The nucleic acid sequence of AvinDRAFT_{—}5579 from Azotobacter vinelandii, e.g. as shown in the respective line in column 5 of Table I, application no. 6, has been published in D. And the activity of the gene product thereof is the activity of peptidase.

Accordingly, in one embodiment, the process of the present invention for producing leucine in a non-human organism, like a microorganism or a plant or a part thereof, comprises increasing or generating the activity of a gene product with the activity of a gene product conferring the activity of “peptidase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said AvinDRAFT_{—}5579, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}5579, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}5579, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}5579, and preferably the activity is increased non-targeted, whereby the respective line discloses in column 7 the fine chemical leucine.

Accordingly, in one embodiment, the molecule which activity is to be increased in the process of the invention is the gene product with an activity as a “peptidase”, preferably it is the molecule of section (a) or (b) of this paragraph.

In particular, it was observed that in plants, especially in Arabidopsis thaliana, increasing or generating the activity of a gene product non-targeted with the activity of a “peptidase”, preferably being encoded by a gene comprising the nucleic acid sequence SEQ ID NO. 74662, preferably the coding region thereof, conferred the production of or the increase in leucine compared with the wild type control.

The nucleic acid sequence of AvinDRAFT_{—}6093 from Azotobacter vinelandii, e.g. as shown in the respective line in column 5 of Table I, application no. 6, has been published in D. And the activity of the gene product thereof is the activity of chaperone protein CIpB.

Accordingly, in one embodiment, the process of the present invention for producing isoleucine in a non-human organism, like a microorganism or a plant or a part thereof, comprises increasing or generating the activity of a gene product with the activity of a gene product conferring the activity of “chaperone protein CIpB”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said AvinDRAFT_{—}6093, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}6093, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}6093, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}6093, and preferably the activity is increased non-targeted, whereby the respective line discloses in column 7 the fine chemical isoleucine.

Accordingly, in one embodiment, the molecule which activity is to be increased in the process of the invention is the gene product with an activity as a “chaperone protein CIpB”, preferably it is the molecule of section (a) or (b) of this paragraph.

In particular, it was observed that in plants, especially in Arabidopsis thaliana, increasing or generating the activity of a gene product non-targeted with the activity of a “chaperone protein CIpB”, preferably being encoded by a gene comprising the nucleic acid sequence SEQ ID NO. 33596, preferably the coding region thereof, conferred the production of or the increase in isoleucine compared with the wild type control.

The nucleic acid sequence of AvinDRAFT_{—}6700 from Azotobacter vinelandii, e.g. as shown in the respective line in column 5 of Table I, application no. 6, has been published in D. And the activity of the gene product thereof is the activity of purine nucleoside phosphorylase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “purine nucleoside phosphorylase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said AvinDRAFT_{—}6700, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}6700, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}6700, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}6700, and preferably the activity is increased non-targeted, whereby the respective line discloses in column 7 the fine chemical isoleucine.

Accordingly, in one embodiment, the molecule which activity is to be increased in the process of the invention is the gene product with an activity as a “purine nucleoside phosphorylase”, preferably it is the molecule of section (a) or (b) of this paragraph.

In particular, it was observed that in plants, especially in Arabidopsis thaliana, increasing or generating the activity of a gene product non-targeted with the activity of a “purine nucleoside phosphorylase”, preferably being encoded by a gene comprising the nucleic acid sequence SEQ ID NO. 34044, preferably the coding region thereof, conferred the production of or the increase in isoleucine compared with the wild type control.

The nucleic acid sequence of AvinDRAFT_{—}6700 from Azotobacter vinelandii, e.g. as shown in the respective line in column 5 of Table I, application no. 6, has been published in D. And the activity of the gene product thereof is the activity of purine nucleoside phosphorylase.

Accordingly, in one embodiment, the process of the present invention for producing leucine in a non-human organism, like a microorganism or a plant or a part thereof, comprises increasing or generating the activity of a gene product with the activity of a gene product conferring the activity of “purine nucleoside phosphorylase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said AvinDRAFT_{—}6700, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}6700, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}6700, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}6700, and preferably the activity is increased non-targeted, whereby the respective line discloses in column 7 the fine chemical leucine.

Accordingly, in one embodiment, the molecule which activity is to be increased in the process of the invention is the gene product with an activity as a “purine nucleoside phosphorylase”, preferably it is the molecule of section (a) or (b) of this paragraph.

In particular, it was observed that in plants, especially in Arabidopsis thaliana, increasing or generating the activity of a gene product non-targeted with the activity of a “purine nucleoside phosphorylase”, preferably being encoded by a gene comprising the nucleic acid sequence SEQ ID NO. 34044, preferably the coding region thereof, conferred the production of or the increase in leucine compared with the wild type control.

The nucleic acid sequence of AvinDRAFT_{—}6700 from Azotobacter vinelandii, e.g. as shown in the respective line in column 5 of Table I, application no. 6, has been published in D. And the activity of the gene product thereof is the activity of purine nucleoside phosphorylase.

Accordingly, in one embodiment, the process of the present invention for producing valine in a non-human organism, like a microorganism or a plant or a part thereof, comprises increasing or generating the activity of a gene product with the activity of a gene product conferring the activity of “purine nucleoside phosphorylase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said AvinDRAFT_{—}6700, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AvinDRAFT_{—}6700, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AvinDRAFT_{—}6700, or a functional equivalent or a homolog thereof as depicted in column 8 of Table II, application no. 6, preferably a homolog or functional equivalent as depicted in column 8 of Table II B, application no. 6, and being depicted in the same respective line as said AvinDRAFT_{—}6700, and preferably the activity is increased non-targeted, whereby the respective line discloses in column 7 the fine chemical valine.

Accordingly, in one embodiment, the molecule which activity is to be increased in the process of the invention is the gene product with an activity as a “purine nucleoside phosphorylase”, preferably it is the molecule of section (a) or (b) of this paragraph.

In particular, it was observed that in plants, especially in Arabidopsis thaliana, increasing or generating the activity of a gene product non-targeted with the activity of a “purine nucleoside phosphorylase”, preferably being encoded by a gene comprising the nucleic acid sequence SEQ ID NO. 34044, preferably the coding region thereof, conferred the production of or the increase in valine compared with the wild type control.

The nucleic acid sequence of AY623894 from Zea mays, e.g. as shown in the respective line in column 5 of Table I, application no. 6, is unpublished. And the activity of the gene product thereof is the activity of pyruvate kinase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “pyruvate kinase”, especially from Zea mays or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said AY623894, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AY623894, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AY623894, or a functional equivalent or a homolog thereof as depicted in column 8 of Table II, application no. 6, preferably a homolog or functional equivalent as depicted in column 8 of Table II B, application no. 6, and being depicted in the same respective line as said AY623894, and preferably the activity is increased non-targeted, whereby the respective line discloses in column 7 the fine chemical isoleucine.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product non-targeted with the activity of a “pyruvate kinase”, preferably being encoded by a gene comprising the nucleic acid sequence SEQ ID NO. 103433, preferably the coding region thereof, conferred the production of or the increase in isoleucine compared with the wild type control.

The nucleic acid sequence of AY623894 from Zea mays, e.g. as shown in the respective line in column 5 of Table I, application no. 6, is unpublished. And the activity of the gene product thereof is the activity of pyruvate kinase.

Accordingly, in one embodiment, the process of the present invention for producing valine in a non-human organism, like a microorganism or a plant or a part thereof, comprises increasing or generating the activity of a gene product with the activity of a gene product conferring the activity of “pyruvate kinase”, especially from Zea mays or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said AY623894, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said AY623894, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said AY623894, or a functional equivalent or a homolog thereof as depicted in column 8 of Table II, application no. 6, preferably a homolog or functional equivalent as depicted in column 8 of Table II B, application no. 6, and being depicted in the same respective line as said AY623894, and preferably the activity is increased non-targeted, whereby the respective line discloses in column 7 the fine chemical valine.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product non-targeted with the activity of a “pyruvate kinase”, preferably being encoded by a gene comprising the nucleic acid sequence SEQ ID NO. 103433, preferably the coding region thereof, conferred the production of or the increase in valine compared with the wild type control.

The nucleic acid sequence of B0004 from Escherichia coli, e.g. as shown in the respective line in column 5 of Table I, application no. 6, has been published in B. And the activity of the gene product thereof is the activity of threonine synthase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “threonine synthase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B0004, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B0004, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B0004, or a functional equivalent or a homolog thereof as depicted in column 8 of Table II, application no. 6, preferably a homolog or functional equivalent as depicted in column 8 of Table II B, application no. 6, and being depicted in the same respective line as said B0004, and preferably the activity is increased plastidic, whereby the respective line discloses in column 7 the fine chemical isoleucine.

Accordingly, in one embodiment, the molecule which activity is to be increased in the process of the invention is the gene product with an activity as a “threonine synthase”, preferably it is the molecule of section (a) or (b) of this paragraph.

In particular, it was observed that in plants, especially in Arabidopsis thaliana, increasing or generating the activity of a gene product plastidic with the activity of a “threonine synthase”, preferably being encoded by a gene comprising the nucleic acid sequence SEQ ID NO. 34889, preferably the coding region thereof, conferred the production of or the increase in isoleucine compared with the wild type control.

The nucleic acid sequence of B0004 from Escherichia coli, e.g. as shown in the respective line in column 5 of Table I, application no. 6, has been published in B. And the activity of the gene product thereof is the activity of threonine synthase.

Accordingly, in one embodiment, the process of the present invention for producing leucine in a non-human organism, like a microorganism or a plant or a part thereof, comprises increasing or generating the activity of a gene product with the activity of a gene product conferring the activity of “threonine synthase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B0004, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B0004, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B0004, or a functional equivalent or a homolog thereof as depicted in column 8 of Table II, application no. 6, preferably a homolog or functional equivalent as depicted in column 8 of Table II B, application no. 6, and being depicted in the same respective line as said B0004, and preferably the activity is increased plastidic, whereby the respective line discloses in column 7 the fine chemical leucine.

Accordingly, in one embodiment, the molecule which activity is to be increased in the process of the invention is the gene product with an activity as a “threonine synthase”, preferably it is the molecule of section (a) or (b) of this paragraph.

In particular, it was observed that in plants, especially in Arabidopsis thaliana, increasing or generating the activity of a gene product plastidic with the activity of a “threonine synthase”, preferably being encoded by a gene comprising the nucleic acid sequence SEQ ID NO. 34889, preferably the coding region thereof, conferred the production of or the increase in leucine compared with the wild type control.

The nucleic acid sequence of B0004 from Escherichia coli, e.g. as shown in the respective line in column 5 of Table I, application no. 6, has been published in B. And the activity of the gene product thereof is the activity of threonine synthase.

Accordingly, in one embodiment, the process of the present invention for producing valine in a non-human organism, like a microorganism or a plant or a part thereof, comprises increasing or generating the activity of a gene product with the activity of a gene product conferring the activity of “threonine synthase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B0004, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B0004, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B0004, or a functional equivalent or a homolog thereof as depicted in column 8 of Table II, application no. 6, preferably a homolog or functional equivalent as depicted in column 8 of Table II B, application no. 6, and being depicted in the same respective line as said B0004, and preferably the activity is increased plastidic, whereby the respective line discloses in column 7 the fine chemical valine.

Accordingly, in one embodiment, the molecule which activity is to be increased in the process of the invention is the gene product with an activity as a “threonine synthase”, preferably it is the molecule of section (a) or (b) of this paragraph.

In particular, it was observed that in plants, especially in Arabidopsis thaliana, increasing or generating the activity of a gene product plastidic with the activity of a “threonine synthase”, preferably being encoded by a gene comprising the nucleic acid sequence SEQ ID NO. 34889, preferably the coding region thereof, conferred the production of or the increase in valine compared with the wild type control.

The nucleic acid sequence of B0050 from Escherichia coli, e.g. as shown in the respective line in column 5 of Table I, application no. 6, has been published in B. And the activity of the gene product thereof is the activity of b0050-protein.

Accordingly, in one embodiment, the process of the present invention for producing valine in a non-human organism, like a microorganism or a plant or a part thereof, comprises increasing or generating the activity of a gene product with the activity of a gene product conferring the activity of “b0050-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B0050, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B0050, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B0050, or a functional equivalent or a homolog thereof as depicted in column 8 of Table II, application no. 6, preferably a homolog or functional equivalent as depicted in column 8 of Table II B, application no. 6, and being depicted in the same respective line as said B0050, and preferably the activity is increased non-targeted, whereby the respective line discloses in column 7 the fine chemical valine.

Accordingly, in one embodiment, the molecule which activity is to be increased in the process of the invention is the gene product with an activity as a “b0050-protein”, preferably it is the molecule of section (a) or (b) of this paragraph.

In particular, it was observed that in plants, especially in Arabidopsis thaliana, increasing or generating the activity of a gene product non-targeted with the activity of a “b0050-protein”, preferably being encoded by a gene comprising the nucleic acid sequence SEQ ID NO. 103958, preferably the coding region thereof, conferred the production of or the increase in valine compared with the wild type control.

The nucleic acid sequence of B0078 from Escherichia coli, e.g. as shown in the respective line in column 5 of Table I, application no. 6, has been published in B. And the activity of the gene product thereof is the activity of acetolactate synthase small subunit.

Accordingly, in one embodiment, the process of the present invention for producing leucine in a non-human organism, like a microorganism or a plant or a part thereof, comprises increasing or generating the activity of a gene product with the activity of a gene product conferring the activity of “acetolactate synthase small subunit”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B0078, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B0078, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B0078, or a functional equivalent or a homolog thereof as depicted in column 8 of Table II, application no. 6, preferably a homolog or functional equivalent as depicted in column 8 of Table II B, application no. 6, and being depicted in the same respective line as said B0078, and preferably the activity is increased non-targeted, whereby the respective line discloses in column 7 the fine chemical leucine.

Accordingly, in one embodiment, the molecule which activity is to be increased in the process of the invention is the gene product with an activity as a “acetolactate synthase small subunit”, preferably it is the molecule of section (a) or (b) of this paragraph.

In particular, it was observed that in plants, especially in Arabidopsis thaliana, increasing or generating the activity of a gene product non-targeted with the activity of a “acetolactate synthase small subunit”, preferably being encoded by a gene comprising the nucleic acid sequence SEQ ID NO. 6818, preferably the coding region thereof, conferred the production of or the increase in leucine compared with the wild type control.

The nucleic acid sequence of B0344 from Escherichia coli, e.g. as shown in the respective line in column 5 of Table I, application no. 6, has been published in B. And the activity of the gene product thereof is the activity of beta-galactosidase.

Accordingly, in one embodiment, the process of the present invention for producing leucine in a non-human organism, like a microorganism or a plant or a part thereof, comprises increasing or generating the activity of a gene product with the activity of a gene product conferring the activity of “beta-galactosidase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B0344, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B0344, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B0344, or a functional equivalent or a homolog thereof as depicted in column 8 of Table II, application no. 6, preferably a homolog or functional equivalent as depicted in column 8 of Table II B, application no. 6, and being depicted in the same respective line as said B0344, and preferably the activity is increased plastidic, whereby the respective line discloses in column 7 the fine chemical leucine.

Accordingly, in one embodiment, the molecule which activity is to be increased in the process of the invention is the gene product with an activity as a “beta-galactosidase”, preferably it is the molecule of section (a) or (b) of this paragraph.

In particular, it was observed that in plants, especially in Arabidopsis thaliana, increasing or generating the activity of a gene product plastidic with the activity of a “beta-galactosidase”, preferably being encoded by a gene comprising the nucleic acid sequence SEQ ID NO. 35733, preferably the coding region thereof, conferred the production of or the increase in leucine compared with the wild type control.

The nucleic acid sequence of B0348 from Escherichia coli, e.g. as shown in the respective line in column 5 of Table I, application no. 6, has been published in B. And the activity of the gene product thereof is the activity of 2,3-dihydroxyphenylpropionate 1,2-dioxygenase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “2,3-dihydroxyphenylpropionate 1,2-dioxygenase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B0348, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B0348, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B0348, or a functional equivalent or a homolog thereof as depicted in column 8 of Table II, application no. 6, preferably a homolog or functional equivalent as depicted in column 8 of Table II B, application no. 6, and being depicted in the same respective line as said B0348, and preferably the activity is increased plastidic, whereby the respective line discloses in column 7 the fine chemical isoleucine.

Accordingly, in one embodiment, the molecule which activity is to be increased in the process of the invention is the gene product with an activity as a “2,3-dihydroxyphenylpropionate 1,2-dioxygenase”, preferably it is the molecule of section (a) or (b) of this paragraph.

In particular, it was observed that in plants, especially in Arabidopsis thaliana, increasing or generating the activity of a gene product plastidic with the activity of a “2,3-dihydroxyphenylpropionate 1,2-dioxygenase”, preferably being encoded by a gene comprising the nucleic acid sequence SEQ ID NO. 7269, preferably the coding region thereof, conferred the production of or the increase in isoleucine compared with the wild type control.

The nucleic acid sequence of B0348 from Escherichia coli, e.g. as shown in the respective line in column 5 of Table I, application no. 6, has been published in B. And the activity of the gene product thereof is the activity of 2,3-dihydroxyphenylpropionate 1,2-dioxygenase.

Accordingly, in one embodiment, the process of the present invention for producing valine in a non-human organism, like a microorganism or a plant or a part thereof, comprises increasing or generating the activity of a gene product with the activity of a gene product conferring the activity of “2,3-dihydroxyphenylpropionate 1,2-dioxygenase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B0348, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B0348, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B0348, or a functional equivalent or a homolog thereof as depicted in column 8 of Table II, application no. 6, preferably a homolog or functional equivalent as depicted in column 8 of Table II B, application no. 6, and being depicted in the same respective line as said B0348, and preferably the activity is increased plastidic, whereby the respective line discloses in column 7 the fine chemical valine.

Accordingly, in one embodiment, the molecule which activity is to be increased in the process of the invention is the gene product with an activity as a “2,3-dihydroxyphenylpropionate 1,2-dioxygenase”, preferably it is the molecule of section (a) or (b) of this paragraph.

In particular, it was observed that in plants, especially in Arabidopsis thaliana, increasing or generating the activity of a gene product plastidic with the activity of a “2,3-dihydroxyphenylpropionate 1,2-dioxygenase”, preferably being encoded by a gene comprising the nucleic acid sequence SEQ ID NO. 7269, preferably the coding region thereof, conferred the production of or the increase in valine compared with the wild type control.

The nucleic acid sequence of B0449 from Escherichia coli, e.g. as shown in the respective line in column 5 of Table I, application no. 6, has been published in B. And the activity of the gene product thereof is the activity of ATP-binding component of a transport system.

Accordingly, in one embodiment, the process of the present invention for producing isoleucine in a non-human organism, like a microorganism or a plant or a part thereof, comprises increasing or generating the activity of a gene product with the activity of a gene product conferring the activity of “ATP-binding component of a transport system”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B0449, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B0449, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B0449, or a functional equivalent or a homolog thereof as depicted in column 8 of Table II, application no. 6, preferably a homolog or functional equivalent as depicted in column 8 of Table II B, application no. 6, and being depicted in the same respective line as said B0449, and preferably the activity is increased non-targeted, whereby the respective line discloses in column 7 the fine chemical isoleucine.

Accordingly, in one embodiment, the molecule which activity is to be increased in the process of the invention is the gene product with an activity as a “ATP-binding component of a transport system”, preferably it is the molecule of section (a) or (b) of this paragraph.

In particular, it was observed that in plants, especially in Arabidopsis thaliana, increasing or generating the activity of a gene product non-targeted with the activity of a “ATP-binding component of a transport system”, preferably being encoded by a gene comprising the nucleic acid sequence SEQ ID NO. 7333, preferably the coding region thereof, conferred the production of or the increase in isoleucine compared with the wild type control.

The nucleic acid sequence of B0449 from Escherichia coli, e.g. as shown in the respective line in column 5 of Table I, application no. 6, has been published in B. And the activity of the gene product thereof is the activity of ATP-binding component of a transport system.

Accordingly, in one embodiment, the process of the present invention for producing leucine in a non-human organism, like a microorganism or a plant or a part thereof, comprises increasing or generating the activity of a gene product with the activity of a gene product conferring the activity of “ATP-binding component of a transport system”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B0449, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B0449, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B0449, or a functional equivalent or a homolog thereof as depicted in column 8 of Table II, application no. 6, preferably a homolog or functional equivalent as depicted in column 8 of Table II B, application no. 6, and being depicted in the same respective line as said B0449, and preferably the activity is increased non-targeted, whereby the respective line discloses in column 7 the fine chemical leucine.

Accordingly, in one embodiment, the molecule which activity is to be increased in the process of the invention is the gene product with an activity as a “ATP-binding component of a transport system”, preferably it is the molecule of section (a) or (b) of this paragraph.

In particular, it was observed that in plants, especially in Arabidopsis thaliana, increasing or generating the activity of a gene product non-targeted with the activity of a “ATP-binding component of a transport system”, preferably being encoded by a gene comprising the nucleic acid sequence SEQ ID NO. 7333, preferably the coding region thereof, conferred the production of or the increase in leucine compared with the wild type control.

The nucleic acid sequence of B0449 from Escherichia coli, e.g. as shown in the respective line in column 5 of Table I, application no. 6, has been published in B. And the activity of the gene product thereof is the activity of ATP-binding component of a transport system.

Accordingly, in one embodiment, the process of the present invention for producing valine in a non-human organism, like a microorganism or a plant or a part thereof, comprises increasing or generating the activity of a gene product with the activity of a gene product conferring the activity of “ATP-binding component of a transport system”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B0449, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B0449, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B0449, or a functional equivalent or a homolog thereof as depicted in column 8 of Table II, application no. 6, preferably a homolog or functional equivalent as depicted in column 8 of Table II B, application no. 6, and being depicted in the same respective line as said B0449, and preferably the activity is increased non-targeted, whereby the respective line discloses in column 7 the fine chemical valine.

Accordingly, in one embodiment, the molecule which activity is to be increased in the process of the invention is the gene product with an activity as a “ATP-binding component of a transport system”, preferably it is the molecule of section (a) or (b) of this paragraph.

In particular, it was observed that in plants, especially in Arabidopsis thaliana, increasing or generating the activity of a gene product non-targeted with the activity of a “ATP-binding component of a transport system”, preferably being encoded by a gene comprising the nucleic acid sequence SEQ ID NO. 7333, preferably the coding region thereof, conferred the production of or the increase in valine compared with the wild type control.

The nucleic acid sequence of B0486 from Escherichia coli, e.g. as shown in the respective line in column 5 of Table I, application no. 6, has been published in B. And the activity of the gene product thereof is the activity of membrane transport protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “membrane transport protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B0486, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B0486, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B0486, or a functional equivalent or a homolog thereof as depicted in column 8 of Table II, application no. 6, preferably a homolog or functional equivalent as depicted in column 8 of Table II B, application no. 6, and being depicted in the same respective line as said B0486, and preferably the activity is increased non-targeted, whereby the respective line discloses in column 7 the fine chemical isoleucine.

Accordingly, in one embodiment, the molecule which activity is to be increased in the process of the invention is the gene product with an activity as a “membrane transport protein”, preferably it is the molecule of section (a) or (b) of this paragraph.

In particular, it was observed that in plants, especially in Arabidopsis thaliana, increasing or generating the activity of a gene product non-targeted with the activity of a “membrane transport protein”, preferably being encoded by a gene comprising the nucleic acid sequence SEQ ID NO. 7686, preferably the coding region thereof, conferred the production of or the increase in isoleucine compared with the wild type control.

The nucleic acid sequence of B0593 from Escherichia coli, e.g. as shown in the respective line in column 5 of Table I, application no. 6, has been published in B. And the activity of the gene product thereof is the activity of isochorismate synthase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “isochorismate synthase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B0593, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B0593, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B0593, or a functional equivalent or a homolog thereof as depicted in column 8 of Table II, application no. 6, preferably a homolog or functional equivalent as depicted in column 8 of Table II B, application no. 6, and being depicted in the same respective line as said B0593, and preferably the activity is increased plastidic, whereby the respective line discloses in column 7 the fine chemical isoleucine.

Accordingly, in one embodiment, the molecule which activity is to be increased in the process of the invention is the gene product with an activity as a “isochorismate synthase”, preferably it is the molecule of section (a) or (b) of this paragraph.

In particular, it was observed that in plants, especially in Arabidopsis thaliana, increasing or generating the activity of a gene product plastidic with the activity of a “isochorismate synthase”, preferably being encoded by a gene comprising the nucleic acid sequence SEQ ID NO. 35967, preferably the coding region thereof, conferred the production of or the increase in isoleucine compared with the wild type control.

The nucleic acid sequence of B0593 from Escherichia coli, e.g. as shown in the respective line in column 5 of Table I, application no. 6, has been published in B. And the activity of the gene product thereof is the activity of isochorismate synthase.

Accordingly, in one embodiment, the process of the present invention for producing leucine in a non-human organism, like a microorganism or a plant or a part thereof, comprises increasing or generating the activity of a gene product with the activity of a gene product conferring the activity of “isochorismate synthase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B0593, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B0593, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B0593, or a functional equivalent or a homolog thereof as depicted in column 8 of Table II, application no. 6, preferably a homolog or functional equivalent as depicted in column 8 of Table II B, application no. 6, and being depicted in the same respective line as said B0593, and preferably the activity is increased plastidic, whereby the respective line discloses in column 7 the fine chemical leucine.

Accordingly, in one embodiment, the molecule which activity is to be increased in the process of the invention is the gene product with an activity as a “isochorismate synthase”, preferably it is the molecule of section (a) or (b) of this paragraph.

In particular, it was observed that in plants, especially in Arabidopsis thaliana, increasing or generating the activity of a gene product plastidic with the activity of a “isochorismate synthase”, preferably being encoded by a gene comprising the nucleic acid sequence SEQ ID NO. 35967, preferably the coding region thereof, conferred the production of or the increase in leucine compared with the wild type control.

The nucleic acid sequence of B0593 from Escherichia coli, e.g. as shown in the respective line in column 5 of Table I, application no. 6, has been published in B. And the activity of the gene product thereof is the activity of isochorismate synthase.

Accordingly, in one embodiment, the process of the present invention for producing valine in a non-human organism, like a microorganism or a plant or a part thereof, comprises increasing or generating the activity of a gene product with the activity of a gene product conferring the activity of “isochorismate synthase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B0593, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B0593, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B0593, or a functional equivalent or a homolog thereof as depicted in column 8 of Table II, application no. 6, preferably a homolog or functional equivalent as depicted in column 8 of Table II B, application no. 6, and being depicted in the same respective line as said B0593, and preferably the activity is increased plastidic, whereby the respective line discloses in column 7 the fine chemical valine.

Accordingly, in one embodiment, the molecule which activity is to be increased in the process of the invention is the gene product with an activity as a “isochorismate synthase”, preferably it is the molecule of section (a) or (b) of this paragraph.

In particular, it was observed that in plants, especially in Arabidopsis thaliana, increasing or generating the activity of a gene product plastidic with the activity of a “isochorismate synthase”, preferably being encoded by a gene comprising the nucleic acid sequence SEQ ID NO. 35967, preferably the coding region thereof, conferred the production of or the increase in valine compared with the wild type control.

The nucleic acid sequence of B0752 from Escherichia coli, e.g. as shown in the respective line in column 5 of Table I, application no. 6, has been published in B. And the activity of the gene product thereof is the activity of zinc transporter.

Accordingly, in one embodiment, the process of the present invention for producing isoleucine in a non-human organism, like a microorganism or a plant or a part thereof, comprises increasing or generating the activity of a gene product with the activity of a gene product conferring the activity of “zinc transporter”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B0752, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B0752, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B0752, or a functional equivalent or a homolog thereof as depicted in column 8 of Table II, application no. 6, preferably a homolog or functional equivalent as depicted in column 8 of Table II B, application no. 6, and being depicted in the same respective line as said B0752, and preferably the activity is increased non-targeted, whereby the respective line discloses in column 7 the fine chemical isoleucine.

Accordingly, in one embodiment, the molecule which activity is to be increased in the process of the invention is the gene product with an activity as a “zinc transporter”, preferably it is the molecule of section (a) or (b) of this paragraph.

In particular, it was observed that in plants, especially in Arabidopsis thaliana, increasing or generating the activity of a gene product non-targeted with the activity of a “zinc transporter”, preferably being encoded by a gene comprising the nucleic acid sequence SEQ ID NO. 36114, preferably the coding region thereof, conferred the production of or the increase in isoleucine compared with the wild type control.

The nucleic acid sequence of B0752 from Escherichia coli, e.g. as shown in the respective line in column 5 of Table I, application no. 6, has been published in B. And the activity of the gene product thereof is the activity of zinc transporter.

Accordingly, in one embodiment, the process of the present invention for producing leucine in a non-human organism, like a microorganism or a plant or a part thereof, comprises increasing or generating the activity of a gene product with the activity of a gene product conferring the activity of “zinc transporter”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B0752, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B0752, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B0752, or a functional equivalent or a homolog thereof as depicted in column 8 of Table II, application no. 6, preferably a homolog or functional equivalent as depicted in column 8 of Table II B, application no. 6, and being depicted in the same respective line as said B0752, and preferably the activity is increased non-targeted, whereby the respective line discloses in column 7 the fine chemical leucine.

Accordingly, in one embodiment, the molecule which activity is to be increased in the process of the invention is the gene product with an activity as a “zinc transporter”, preferably it is the molecule of section (a) or (b) of this paragraph.

In particular, it was observed that in plants, especially in Arabidopsis thaliana, increasing or generating the activity of a gene product non-targeted with the activity of a “zinc transporter”, preferably being encoded by a gene comprising the nucleic acid sequence SEQ ID NO. 36114, preferably the coding region thereof, conferred the production of or the increase in leucine compared with the wild type control.

The nucleic acid sequence of B0898 from Escherichia coli, e.g. as shown in the respective line in column 5 of Table I, application no. 6, has been published in B. And the activity of the gene product thereof is the activity of major facilitator superfamily transporter protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “major facilitator superfamily transporter protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B0898, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B0898, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B0898, or a functional equivalent or a homolog thereof as depicted in column 8 of Table II, application no. 6, preferably a homolog or functional equivalent as depicted in column 8 of Table II B, application no. 6, and being depicted in the same respective line as said B0898, and preferably the activity is increased non-targeted, whereby the respective line discloses in column 7 the fine chemical isoleucine.

Accordingly, in one embodiment, the molecule which activity is to be increased in the process of the invention is the gene product with an activity as a “major facilitator superfamily transporter protein”, preferably it is the molecule of section (a) or (b) of this paragraph.

In particular, it was observed that in plants, especially in Arabidopsis thaliana, increasing or generating the activity of a gene product non-targeted with the activity of a “major facilitator superfamily transporter protein”, preferably being encoded by a gene comprising the nucleic acid sequence SEQ ID NO. 7917, preferably the coding region thereof, conferred the production of or the increase in isoleucine compared with the wild type control.

The nucleic acid sequence of B0898 from Escherichia coli, e.g. as shown in the respective line in column 5 of Table I, application no. 6, has been published in B. And the activity of the gene product thereof is the activity of major facilitator superfamily transporter protein.

Accordingly, in one embodiment, the process of the present invention for producing leucine in a non-human organism, like a microorganism or a plant or a part thereof, comprises increasing or generating the activity of a gene product with the activity of a gene product conferring the activity of “major facilitator superfamily transporter protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B0898, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B0898, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B0898, or a functional equivalent or a homolog thereof as depicted in column 8 of Table II, application no. 6, preferably a homolog or functional equivalent as depicted in column 8 of Table II B, application no. 6, and being depicted in the same respective line as said B0898, and preferably the activity is increased non-targeted, whereby the respective line discloses in column 7 the fine chemical leucine.

Accordingly, in one embodiment, the molecule which activity is to be increased in the process of the invention is the gene product with an activity as a “major facilitator superfamily transporter protein”, preferably it is the molecule of section (a) or (b) of this paragraph.

In particular, it was observed that in plants, especially in Arabidopsis thaliana, increasing or generating the activity of a gene product non-targeted with the activity of a “major facilitator superfamily transporter protein”, preferably being encoded by a gene comprising the nucleic acid sequence SEQ ID NO. 7917, preferably the coding region thereof, conferred the production of or the increase in leucine compared with the wild type control.

The nucleic acid sequence of B0898 from Escherichia coli, e.g. as shown in the respective line in column 5 of Table I, application no. 6, has been published in B. And the activity of the gene product thereof is the activity of major facilitator superfamily transporter protein.

Accordingly, in one embodiment, the process of the present invention for producing valine in a non-human organism, like a microorganism or a plant or a part thereof, comprises increasing or generating the activity of a gene product with the activity of a gene product conferring the activity of “major facilitator superfamily transporter protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B0898, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B0898, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B0898, or a functional equivalent or a homolog thereof as depicted in column 8 of Table II, application no. 6, preferably a homolog or functional equivalent as depicted in column 8 of Table II B, application no. 6, and being depicted in the same respective line as said B0898, and preferably the activity is increased non-targeted, whereby the respective line discloses in column 7 the fine chemical valine.

Accordingly, in one embodiment, the molecule which activity is to be increased in the process of the invention is the gene product with an activity as a “major facilitator superfamily transporter protein”, preferably it is the molecule of section (a) or (b) of this paragraph.

In particular, it was observed that in plants, especially in Arabidopsis thaliana, increasing or generating the activity of a gene product non-targeted with the activity of a “major facilitator superfamily transporter protein”, preferably being encoded by a gene comprising the nucleic acid sequence SEQ ID NO. 7917, preferably the coding region thereof, conferred the production of or the increase in valine compared with the wild type control.

The nucleic acid sequence of B0962 from Escherichia coli, e.g. as shown in the respective line in column 5 of Table I, application no. 6, has been published in B. And the activity of the gene product thereof is the activity of DNA helicase IV.

Accordingly, in one embodiment, the process of the present invention for producing leucine in a non-human organism, like a microorganism or a plant or a part thereof, comprises increasing or generating the activity of a gene product with the activity of a gene product conferring the activity of “DNA helicase IV”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B0962, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B0962, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B0962, or a functional equivalent or a homolog thereof as depicted in column 8 of Table II, application no. 6, preferably a homolog or functional equivalent as depicted in column 8 of Table II B, application no. 6, and being depicted in the same respective line as said B0962, and preferably the activity is increased non-targeted, whereby the respective line discloses in column 7 the fine chemical leucine.

Accordingly, in one embodiment, the molecule which activity is to be increased in the process of the invention is the gene product with an activity as a “DNA helicase IV”, preferably it is the molecule of section (a) or (b) of this paragraph.

In particular, it was observed that in plants, especially in Arabidopsis thaliana, increasing or generating the activity of a gene product non-targeted with the activity of a “DNA helicase IV”, preferably being encoded by a gene comprising the nucleic acid sequence SEQ ID NO. 36623, preferably the coding region thereof, conferred the production of or the increase in leucine compared with the wild type control.

The nucleic acid sequence of B0963 from Escherichia coli, e.g. as shown in the respective line in column 5 of Table I, application no. 6, has been published in B. And the activity of the gene product thereof is the activity of methylglyoxal synthase.

Accordingly, in one embodiment, the process of the present invention for producing valine in a non-human organism, like a microorganism or a plant or a part thereof, comprises increasing or generating the activity of a gene product with the activity of a gene product conferring the activity of “methylglyoxal synthase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B0963, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B0963, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B0963, or a functional equivalent or a homolog thereof as depicted in column 8 of Table II, application no. 6, preferably a homolog or functional equivalent as depicted in column 8 of Table II B, application no. 6, and being depicted in the same respective line as said B0963, and preferably the activity is increased non-targeted, whereby the respective line discloses in column 7 the fine chemical valine.

Accordingly, in one embodiment, the molecule which activity is to be increased in the process of the invention is the gene product with an activity as a “methylglyoxal synthase”, preferably it is the molecule of section (a) or (b) of this paragraph.

In particular, it was observed that in plants, especially in Arabidopsis thaliana, increasing or generating the activity of a gene product non-targeted with the activity of a “methylglyoxal synthase”, preferably being encoded by a gene comprising the nucleic acid sequence SEQ ID NO. 36670, preferably the coding region thereof, conferred the production of or the increase in valine compared with the wild type control.

The nucleic acid sequence of B0980 from Escherichia coli, e.g. as shown in the respective line in column 5 of Table I, application no. 6, has been published in B. And the activity of the gene product thereof is the activity of phosphoanhydride phosphorylase.

Accordingly, in one embodiment, the process of the present invention for producing valine in a non-human organism, like a microorganism or a plant or a part thereof, comprises increasing or generating the activity of a gene product with the activity of a gene product conferring the activity of “phosphoanhydride phosphorylase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B0980, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B0980, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B0980, or a functional equivalent or a homolog thereof as depicted in column 8 of Table II, application no. 6, preferably a homolog or functional equivalent as depicted in column 8 of Table II B, application no. 6, and being depicted in the same respective line as said B0980, and preferably the activity is increased non-targeted, whereby the respective line discloses in column 7 the fine chemical valine.

Accordingly, in one embodiment, the molecule which activity is to be increased in the process of the invention is the gene product with an activity as a “phosphoanhydride phosphorylase”, preferably it is the molecule of section (a) or (b) of this paragraph.

In particular, it was observed that in plants, especially in Arabidopsis thaliana, increasing or generating the activity of a gene product non-targeted with the activity of a “phosphoanhydride phosphorylase”, preferably being encoded by a gene comprising the nucleic acid sequence SEQ ID NO. 36809, preferably the coding region thereof, conferred the production of or the increase in valine compared with the wild type control.

The nucleic acid sequence of B1003 from Escherichia coli, e.g. as shown in the respective line in column 5 of Table I, application no. 6, has been published in B. And the activity of the gene product thereof is the activity of b1003-protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “b1003-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B1003, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1003, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1003, or a functional equivalent or a homolog thereof as depicted in column 8 of Table II, application no. 6, preferably a homolog or functional equivalent as depicted in column 8 of Table II B, application no. 6, and being depicted in the same respective line as said B1003, and preferably the activity is increased non-targeted, whereby the respective line discloses in column 7 the fine chemical isoleucine.

Accordingly, in one embodiment, the molecule which activity is to be increased in the process of the invention is the gene product with an activity as a “b1003-protein”, preferably it is the molecule of section (a) or (b) of this paragraph.

In particular, it was observed that in plants, especially in Arabidopsis thaliana, increasing or generating the activity of a gene product non-targeted with the activity of a “b1003-protein”, preferably being encoded by a gene comprising the nucleic acid sequence SEQ ID NO. 7941, preferably the coding region thereof, conferred the production of or the increase in isoleucine compared with the wild type control.

The nucleic acid sequence of B1003 from Escherichia coli, e.g. as shown in the respective line in column 5 of Table I, application no. 6, has been published in B. And the activity of the gene product thereof is the activity of b1003-protein.

Accordingly, in one embodiment, the process of the present invention for producing leucine in a non-human organism, like a microorganism or a plant or a part thereof, comprises increasing or generating the activity of a gene product with the activity of a gene product conferring the activity of “b1003-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B1003, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1003, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1003, or a functional equivalent or a homolog thereof as depicted in column 8 of Table II, application no. 6, preferably a homolog or functional equivalent as depicted in column 8 of Table II B, application no. 6, and being depicted in the same respective line as said B1003, and preferably the activity is increased non-targeted, whereby the respective line discloses in column 7 the fine chemical leucine.

Accordingly, in one embodiment, the molecule which activity is to be increased in the process of the invention is the gene product with an activity as a “b1003-protein”, preferably it is the molecule of section (a) or (b) of this paragraph.

In particular, it was observed that in plants, especially in Arabidopsis thaliana, increasing or generating the activity of a gene product non-targeted with the activity of a “b1003-protein”, preferably being encoded by a gene comprising the nucleic acid sequence SEQ ID NO. 7941, preferably the coding region thereof, conferred the production of or the increase in leucine compared with the wild type control.

The nucleic acid sequence of B1003 from Escherichia coli, e.g. as shown in the respective line in column 5 of Table I, application no. 6, has been published in B. And the activity of the gene product thereof is the activity of b1003-protein.

Accordingly, in one embodiment, the process of the present invention for producing valine in a non-human organism, like a microorganism or a plant or a part thereof, comprises increasing or generating the activity of a gene product with the activity of a gene product conferring the activity of “b1003-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B1003, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1003, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1003, or a functional equivalent or a homolog thereof as depicted in column 8 of Table II, application no. 6, preferably a homolog or functional equivalent as depicted in column 8 of Table II B, application no. 6, and being depicted in the same respective line as said B1003, and preferably the activity is increased non-targeted, whereby the respective line discloses in column 7 the fine chemical valine.

Accordingly, in one embodiment, the molecule which activity is to be increased in the process of the invention is the gene product with an activity as a “b1003-protein”, preferably it is the molecule of section (a) or (b) of this paragraph.

In particular, it was observed that in plants, especially in Arabidopsis thaliana, increasing or generating the activity of a gene product non-targeted with the activity of a “b1003-protein”, preferably being encoded by a gene comprising the nucleic acid sequence SEQ ID NO. 7941, preferably the coding region thereof, conferred the production of or the increase in valine compared with the wild type control.

The nucleic acid sequence of B1163 from Escherichia coli, e.g. as shown in the respective line in column 5 of Table I, application no. 6, has been published in B. And the activity of the gene product thereof is the activity of b1163-protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “b1163-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B1163, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1163, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1163, or a functional equivalent or a homolog thereof as depicted in column 8 of Table II, application no. 6, preferably a homolog or functional equivalent as depicted in column 8 of Table II B, application no. 6, and being depicted in the same respective line as said B1163, and preferably the activity is increased non-targeted, whereby the respective line discloses in column 7 the fine chemical isoleucine.

Accordingly, in one embodiment, the molecule which activity is to be increased in the process of the invention is the gene product with an activity as a “b1163-protein”, preferably it is the molecule of section (a) or (b) of this paragraph.

In particular, it was observed that in plants, especially in Arabidopsis thaliana, increasing or generating the activity of a gene product non-targeted with the activity of a “b1163-protein”, preferably being encoded by a gene comprising the nucleic acid sequence SEQ ID NO. 37394, preferably the coding region thereof, conferred the production of or the increase in isoleucine compared with the wild type control.

The nucleic acid sequence of B1163 from Escherichia coli, e.g. as shown in the respective line in column 5 of Table I, application no. 6, has been published in B. And the activity of the gene product thereof is the activity of b1163-protein.

Accordingly, in one embodiment, the process of the present invention for producing leucine in a non-human organism, like a microorganism or a plant or a part thereof, comprises increasing or generating the activity of a gene product with the activity of a gene product conferring the activity of “b1163-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B1163, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1163, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1163, or a functional equivalent or a homolog thereof as depicted in column 8 of Table II, application no. 6, preferably a homolog or functional equivalent as depicted in column 8 of Table II B, application no. 6, and being depicted in the same respective line as said B1163, and preferably the activity is increased non-targeted, whereby the respective line discloses in column 7 the fine chemical leucine.

Accordingly, in one embodiment, the molecule which activity is to be increased in the process of the invention is the gene product with an activity as a “b1163-protein”, preferably it is the molecule of section (a) or (b) of this paragraph.

In particular, it was observed that in plants, especially in Arabidopsis thaliana, increasing or generating the activity of a gene product non-targeted with the activity of a “b1163-protein”, preferably being encoded by a gene comprising the nucleic acid sequence SEQ ID NO. 37394, preferably the coding region thereof, conferred the production of or the increase in leucine compared with the wild type control.

The nucleic acid sequence of B1163 from Escherichia coli, e.g. as shown in the respective line in column 5 of Table I, application no. 6, has been published in B. And the activity of the gene product thereof is the activity of b1163-protein.

Accordingly, in one embodiment, the process of the present invention for producing valine in a non-human organism, like a microorganism or a plant or a part thereof, comprises increasing or generating the activity of a gene product with the activity of a gene product conferring the activity of “b1163-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B1163, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1163, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1163, or a functional equivalent or a homolog thereof as depicted in column 8 of Table II, application no. 6, preferably a homolog or functional equivalent as depicted in column 8 of Table II B, application no. 6, and being depicted in the same respective line as said B1163, and preferably the activity is increased non-targeted, whereby the respective line discloses in column 7 the fine chemical valine.

Accordingly, in one embodiment, the molecule which activity is to be increased in the process of the invention is the gene product with an activity as a “b1163-protein”, preferably it is the molecule of section (a) or (b) of this paragraph.

In particular, it was observed that in plants, especially in Arabidopsis thaliana, increasing or generating the activity of a gene product non-targeted with the activity of a “b1163-protein”, preferably being encoded by a gene comprising the nucleic acid sequence SEQ ID NO. 37394, preferably the coding region thereof, conferred the production of or the increase in valine compared with the wild type control.

The nucleic acid sequence of B1184 from Escherichia coli, e.g. as shown in the respective line in column 5 of Table I, application no. 6, has been published in B. And the activity of the gene product thereof is the activity of DNA polymerase.

Accordingly, in one embodiment, the process of the present invention for producing leucine in a non-human organism, like a microorganism or a plant or a part thereof, comprises increasing or generating the activity of a gene product with the activity of a gene product conferring the activity of “DNA polymerase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B1184, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1184, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1184, or a functional equivalent or a homolog thereof as depicted in column 8 of Table II, application no. 6, preferably a homolog or functional equivalent as depicted in column 8 of Table II B, application no. 6, and being depicted in the same respective line as said B1184, and preferably the activity is increased plastidic, whereby the respective line discloses in column 7 the fine chemical leucine.

Accordingly, in one embodiment, the molecule which activity is to be increased in the process of the invention is the gene product with an activity as a “DNA polymerase”, preferably it is the molecule of section (a) or (b) of this paragraph.

In particular, it was observed that in plants, especially in Arabidopsis thaliana, increasing or generating the activity of a gene product plastidic with the activity of a “DNA polymerase”, preferably being encoded by a gene comprising the nucleic acid sequence SEQ ID NO. 104134, preferably the coding region thereof, conferred the production of or the increase in leucine compared with the wild type control.

The nucleic acid sequence of B1184 from Escherichia coli, e.g. as shown in the respective line in column 5 of Table I, application no. 6, has been published in B. And the activity of the gene product thereof is the activity of DNA polymerase.

Accordingly, in one embodiment, the process of the present invention for producing valine in a non-human organism, like a microorganism or a plant or a part thereof, comprises increasing or generating the activity of a gene product with the activity of a gene product conferring the activity of “DNA polymerase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B1184, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1184, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1184, or a functional equivalent or a homolog thereof as depicted in column 8 of Table II, application no. 6, preferably a homolog or functional equivalent as depicted in column 8 of Table II B, application no. 6, and being depicted in the same respective line as said B1184, and preferably the activity is increased plastidic, whereby the respective line discloses in column 7 the fine chemical valine.

Accordingly, in one embodiment, the molecule which activity is to be increased in the process of the invention is the gene product with an activity as a “DNA polymerase”, preferably it is the molecule of section (a) or (b) of this paragraph.

In particular, it was observed that in plants, especially in Arabidopsis thaliana, increasing or generating the activity of a gene product plastidic with the activity of a “DNA polymerase”, preferably being encoded by a gene comprising the nucleic acid sequence SEQ ID NO. 104134, preferably the coding region thereof, conferred the production of or the increase in valine compared with the wild type control.

The nucleic acid sequence of B1255 from Escherichia coli, e.g. as shown in the respective line in column 5 of Table I, application no. 6, has been published in B. And the activity of the gene product thereof is the activity of membrane protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “membrane protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B1255, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1255, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1255, or a functional equivalent or a homolog thereof as depicted in column 8 of Table II, application no. 6, preferably a homolog or functional equivalent as depicted in column 8 of Table II B, application no. 6, and being depicted in the same respective line as said B1255, and preferably the activity is increased non-targeted, whereby the respective line discloses in column 7 the fine chemical isoleucine.

Accordingly, in one embodiment, the molecule which activity is to be increased in the process of the invention is the gene product with an activity as a “membrane protein”, preferably it is the molecule of section (a) or (b) of this paragraph.

In particular, it was observed that in plants, especially in Arabidopsis thaliana, increasing or generating the activity of a gene product non-targeted with the activity of a “membrane protein”, preferably being encoded by a gene comprising the nucleic acid sequence SEQ ID NO. 37483, preferably the coding region thereof, conferred the production of or the increase in isoleucine compared with the wild type control.

The nucleic acid sequence of B1255 from Escherichia coli, e.g. as shown in the respective line in column 5 of Table I, application no. 6, has been published in B. And the activity of the gene product thereof is the activity of membrane protein.

Accordingly, in one embodiment, the process of the present invention for producing leucine in a non-human organism, like a microorganism or a plant or a part thereof, comprises increasing or generating the activity of a gene product with the activity of a gene product conferring the activity of “membrane protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B1255, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1255, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1255, or a functional equivalent or a homolog thereof as depicted in column 8 of Table II, application no. 6, preferably a homolog or functional equivalent as depicted in column 8 of Table II B, application no. 6, and being depicted in the same respective line as said B1255, and preferably the activity is increased non-targeted, whereby the respective line discloses in column 7 the fine chemical leucine.

Accordingly, in one embodiment, the molecule which activity is to be increased in the process of the invention is the gene product with an activity as a “membrane protein”, preferably it is the molecule of section (a) or (b) of this paragraph.

In particular, it was observed that in plants, especially in Arabidopsis thaliana, increasing or generating the activity of a gene product non-targeted with the activity of a “membrane protein”, preferably being encoded by a gene comprising the nucleic acid sequence SEQ ID NO. 37483, preferably the coding region thereof, conferred the production of or the increase in leucine compared with the wild type control.

The nucleic acid sequence of B1255 from Escherichia coli, e.g. as shown in the respective line in column 5 of Table I, application no. 6, has been published in B. And the activity of the gene product thereof is the activity of membrane protein.

Accordingly, in one embodiment, the process of the present invention for producing valine in a non-human organism, like a microorganism or a plant or a part thereof, comprises increasing or generating the activity of a gene product with the activity of a gene product conferring the activity of “membrane protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B1255, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1255, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1255, or a functional equivalent or a homolog thereof as depicted in column 8 of Table II, application no. 6, preferably a homolog or functional equivalent as depicted in column 8 of Table II B, application no. 6, and being depicted in the same respective line as said B1255, and preferably the activity is increased non-targeted, whereby the respective line discloses in column 7 the fine chemical valine.

Accordingly, in one embodiment, the molecule which activity is to be increased in the process of the invention is the gene product with an activity as a “membrane protein”, preferably it is the molecule of section (a) or (b) of this paragraph.

In particular, it was observed that in plants, especially in Arabidopsis thaliana, increasing or generating the activity of a gene product non-targeted with the activity of a “membrane protein”, preferably being encoded by a gene comprising the nucleic acid sequence SEQ ID NO. 37483, preferably the coding region thereof, conferred the production of or the increase in valine compared with the wild type control.

The nucleic acid sequence of B1263 from Escherichia coli, e.g. as shown in the respective line in column 5 of Table I, application no. 6, has been published in B. And the activity of the gene product thereof is the activity of anthranilate synthase component II.

Accordingly, in one embodiment, the process of the present invention for producing isoleucine in a non-human organism, like a microorganism or a plant or a part thereof, comprises increasing or generating the activity of a gene product with the activity of a gene product conferring the activity of “anthranilate synthase component II”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B1263, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1263, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1263, or a functional equivalent or a homolog thereof as depicted in column 8 of Table II, application no. 6, preferably a homolog or functional equivalent as depicted in column 8 of Table II B, application no. 6, and being depicted in the same respective line as said B1263, and preferably the activity is increased plastidic, whereby the respective line discloses in column 7 the fine chemical isoleucine.

Accordingly, in one embodiment, the molecule which activity is to be increased in the process of the invention is the gene product with an activity as a “anthranilate synthase component II”, preferably it is the molecule of section (a) or (b) of this paragraph.

In particular, it was observed that in plants, especially in Arabidopsis thaliana, increasing or generating the activity of a gene product plastidic with the activity of a “anthranilate synthase component II”, preferably being encoded by a gene comprising the nucleic acid sequence SEQ ID NO. 37539, preferably the coding region thereof, conferred the production of or the increase in isoleucine compared with the wild type control.

The nucleic acid sequence of B1263 from Escherichia coli, e.g. as shown in the respective line in column 5 of Table I, application no. 6, has been published in B. And the activity of the gene product thereof is the activity of anthranilate synthase component II.

Accordingly, in one embodiment, the process of the present invention for producing leucine in a non-human organism, like a microorganism or a plant or a part thereof, comprises increasing or generating the activity of a gene product with the activity of a gene product conferring the activity of “anthranilate synthase component II”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B1263, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1263, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1263, or a functional equivalent or a homolog thereof as depicted in column 8 of Table II, application no. 6, preferably a homolog or functional equivalent as depicted in column 8 of Table II B, application no. 6, and being depicted in the same respective line as said B1263, and preferably the activity is increased plastidic, whereby the respective line discloses in column 7 the fine chemical leucine.

Accordingly, in one embodiment, the molecule which activity is to be increased in the process of the invention is the gene product with an activity as a “anthranilate synthase component II”, preferably it is the molecule of section (a) or (b) of this paragraph.

In particular, it was observed that in plants, especially in Arabidopsis thaliana, increasing or generating the activity of a gene product plastidic with the activity of a “anthranilate synthase component II”, preferably being encoded by a gene comprising the nucleic acid sequence SEQ ID NO. 37539, preferably the coding region thereof, conferred the production of or the increase in leucine compared with the wild type control.

The nucleic acid sequence of B1263 from Escherichia coli, e.g. as shown in the respective line in column 5 of Table I, application no. 6, has been published in B. And the activity of the gene product thereof is the activity of anthranilate synthase component II.

Accordingly, in one embodiment, the process of the present invention for producing valine in a non-human organism, like a microorganism or a plant or a part thereof, comprises increasing or generating the activity of a gene product with the activity of a gene product conferring the activity of “anthranilate synthase component II”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B1263, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1263, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1263, or a functional equivalent or a homolog thereof as depicted in column 8 of Table II, application no. 6, preferably a homolog or functional equivalent as depicted in column 8 of Table II B, application no. 6, and being depicted in the same respective line as said B1263, and preferably the activity is increased plastidic, whereby the respective line discloses in column 7 the fine chemical valine.

Accordingly, in one embodiment, the molecule which activity is to be increased in the process of the invention is the gene product with an activity as a “anthranilate synthase component II”, preferably it is the molecule of section (a) or (b) of this paragraph.

In particular, it was observed that in plants, especially in Arabidopsis thaliana, increasing or generating the activity of a gene product plastidic with the activity of a “anthranilate synthase component II”, preferably being encoded by a gene comprising the nucleic acid sequence SEQ ID NO. 37539, preferably the coding region thereof, conferred the production of or the increase in valine compared with the wild type control.

The nucleic acid sequence of B1297 from Escherichia coli, e.g. as shown in the respective line in column 5 of Table I, application no. 6, has been published in B. And the activity of the gene product thereof is the activity of glutamine synthetase.

Accordingly, in one embodiment, the process of the present invention for producing leucine in a non-human organism, like a microorganism or a plant or a part thereof, comprises increasing or generating the activity of a gene product with the activity of a gene product conferring the activity of “glutamine synthetase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B1297, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1297, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1297, or a functional equivalent or a homolog thereof as depicted in column 8 of Table II, application no. 6, preferably a homolog or functional equivalent as depicted in column 8 of Table II B, application no. 6, and being depicted in the same respective line as said B1297, and preferably the activity is increased plastidic, whereby the respective line discloses in column 7 the fine chemical leucine.

Accordingly, in one embodiment, the molecule which activity is to be increased in the process of the invention is the gene product with an activity as a “glutamine synthetase”, preferably it is the molecule of section (a) or (b) of this paragraph.

In particular, it was observed that in plants, especially in Arabidopsis thaliana, increasing or generating the activity of a gene product plastidic with the activity of a “glutamine synthetase”, preferably being encoded by a gene comprising the nucleic acid sequence SEQ ID NO. 37658, preferably the coding region thereof, conferred the production of or the increase in leucine compared with the wild type control.

The nucleic acid sequence of B1300 from Escherichia coli, e.g. as shown in the respective line in column 5 of Table I, application no. 6, has been published in B. And the activity of the gene product thereof is the activity of aldehyde dehydrogenase.

Accordingly, in one embodiment, the process of the present invention for producing valine in a non-human organism, like a microorganism or a plant or a part thereof, comprises increasing or generating the activity of a gene product with the activity of a gene product conferring the activity of “aldehyde dehydrogenase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B1300, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1300, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1300, or a functional equivalent or a homolog thereof as depicted in column 8 of Table II, application no. 6, preferably a homolog or functional equivalent as depicted in column 8 of Table II B, application no. 6, and being depicted in the same respective line as said B1300, and preferably the activity is increased non-targeted, whereby the respective line discloses in column 7 the fine chemical valine.

Accordingly, in one embodiment, the molecule which activity is to be increased in the process of the invention is the gene product with an activity as a “aldehyde dehydrogenase”, preferably it is the molecule of section (a) or (b) of this paragraph.

In particular, it was observed that in plants, especially in Arabidopsis thaliana, increasing or generating the activity of a gene product non-targeted with the activity of a “aldehyde dehydrogenase”, preferably being encoded by a gene comprising the nucleic acid sequence SEQ ID NO. 37807, preferably the coding region thereof, conferred the production of or the increase in valine compared with the wild type control.

The nucleic acid sequence of B1445 from Escherichia coli, e.g. as shown in the respective line in column 5 of Table I, application no. 6, has been published in B. And the activity of the gene product thereof is the activity of b1445-protein.

Accordingly, in one embodiment, the process of the present invention for producing valine in a non-human organism, like a microorganism or a plant or a part thereof, comprises increasing or generating the activity of a gene product with the activity of a gene product conferring the activity of “b1445-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B1445, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1445, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1445, or a functional equivalent or a homolog thereof as depicted in column 8 of Table II, application no. 6, preferably a homolog or functional equivalent as depicted in column 8 of Table II B, application no. 6, and being depicted in the same respective line as said B1445, and preferably the activity is increased non-targeted, whereby the respective line discloses in column 7 the fine chemical valine.

Accordingly, in one embodiment, the molecule which activity is to be increased in the process of the invention is the gene product with an activity as a “b1445-protein”, preferably it is the molecule of section (a) or (b) of this paragraph.

In particular, it was observed that in plants, especially in Arabidopsis thaliana, increasing or generating the activity of a gene product non-targeted with the activity of a “b1445-protein”, preferably being encoded by a gene comprising the nucleic acid sequence SEQ ID NO. 38289, preferably the coding region thereof, conferred the production of or the increase in valine compared with the wild type control.

The nucleic acid sequence of B1479 from Escherichia coli, e.g. as shown in the respective line in column 5 of Table I, application no. 6, has been published in B. And the activity of the gene product thereof is the activity of NADP-dependent malic enzyme.

Accordingly, in one embodiment, the process of the present invention for producing valine in a non-human organism, like a microorganism or a plant or a part thereof, comprises increasing or generating the activity of a gene product with the activity of a gene product conferring the activity of “NADP-dependent malic enzyme”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B1479, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1479, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1479, or a functional equivalent or a homolog thereof as depicted in column 8 of Table II, application no. 6, preferably a homolog or functional equivalent as depicted in column 8 of Table II B, application no. 6, and being depicted in the same respective line as said B1479, and preferably the activity is increased non-targeted, whereby the respective line discloses in column 7 the fine chemical valine.

Accordingly, in one embodiment, the molecule which activity is to be increased in the process of the invention is the gene product with an activity as a “NADP-dependent malic enzyme”, preferably it is the molecule of section (a) or (b) of this paragraph.

In particular, it was observed that in plants, especially in Arabidopsis thaliana, increasing or generating the activity of a gene product non-targeted with the activity of a “NADP-dependent malic enzyme”, preferably being encoded by a gene comprising the nucleic acid sequence SEQ ID NO. 77774, preferably the coding region thereof, conferred the production of or the increase in valine compared with the wild type control.

The nucleic acid sequence of B1522 from Escherichia coli, e.g. as shown in the respective line in column 5 of Table I, application no. 6, has been published in B. And the activity of the gene product thereof is the activity of b1522-protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “b1522-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B1522, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1522, or a functional equivalent or a homolog thereof as depicted in column 8 of Table II, application no. 6, preferably a homolog or functional equivalent as depicted in column 8 of Table II B, application no. 6, and being depicted in the same respective line as said B1522, and preferably the activity is increased non-targeted, whereby the respective line discloses in column 7 the fine chemical isoleucine.

Accordingly, in one embodiment, the molecule which activity is to be increased in the process of the invention is the gene product with an activity as a “b1522-protein”, preferably it is the molecule of section (a) or (b) of this paragraph.

In particular, it was observed that in plants, especially in Arabidopsis thaliana, increasing or generating the activity of a gene product non-targeted with the activity of a “b1522-protein”, preferably being encoded by a gene comprising the nucleic acid sequence SEQ ID NO. 7947, preferably the coding region thereof, conferred the production of or the increase in isoleucine compared with the wild type control.

The nucleic acid sequence of B1522 from Escherichia coli, e.g. as shown in the respective line in column 5 of Table I, application no. 6, has been published in B. And the activity of the gene product thereof is the activity of b1522-protein.

Accordingly, in one embodiment, the process of the present invention for producing leucine in a non-human organism, like a microorganism or a plant or a part thereof, comprises increasing or generating the activity of a gene product with the activity of a gene product conferring the activity of “b1522-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B1522, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1522, or a functional equivalent or a homolog thereof as depicted in column 8 of Table II, application no. 6, preferably a homolog or functional equivalent as depicted in column 8 of Table II B, application no. 6, and being depicted in the same respective line as said B1522, and preferably the activity is increased non-targeted, whereby the respective line discloses in column 7 the fine chemical leucine.

Accordingly, in one embodiment, the molecule which activity is to be increased in the process of the invention is the gene product with an activity as a “b1522-protein”, preferably it is the molecule of section (a) or (b) of this paragraph.

In particular, it was observed that in plants, especially in Arabidopsis thaliana, increasing or generating the activity of a gene product non-targeted with the activity of a “b1522-protein”, preferably being encoded by a gene comprising the nucleic acid sequence SEQ ID NO. 7947, preferably the coding region thereof, conferred the production of or the increase in leucine compared with the wild type control.

The nucleic acid sequence of B1522 from Escherichia coli, e.g. as shown in the respective line in column 5 of Table I, application no. 6, has been published in B. And the activity of the gene product thereof is the activity of b1522-protein.

Accordingly, in one embodiment, the process of the present invention for producing valine in a non-human organism, like a microorganism or a plant or a part thereof, comprises increasing or generating the activity of a gene product with the activity of a gene product conferring the activity of “b1522-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B1522, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1522, or a functional equivalent or a homolog thereof as depicted in column 8 of Table II, application no. 6, preferably a homolog or functional equivalent as depicted in column 8 of Table II B, application no. 6, and being depicted in the same respective line as said B1522, and preferably the activity is increased non-targeted, whereby the respective line discloses in column 7 the fine chemical valine.

Accordingly, in one embodiment, the molecule which activity is to be increased in the process of the invention is the gene product with an activity as a “b1522-protein”, preferably it is the molecule of section (a) or (b) of this paragraph.

In particular, it was observed that in plants, especially in Arabidopsis thaliana, increasing or generating the activity of a gene product non-targeted with the activity of a “b1522-protein”, preferably being encoded by a gene comprising the nucleic acid sequence SEQ ID NO. 7947, preferably the coding region thereof, conferred the production of or the increase in valine compared with the wild type control.

The nucleic acid sequence of B1597 from Escherichia coli, e.g. as shown in the respective line in column 5 of Table I, application no. 6, has been published in B. And the activity of the gene product thereof is the activity of acid shock protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “acid shock protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B1597, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1597, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1597, or a functional equivalent or a homolog thereof as depicted in column 8 of Table II, application no. 6, preferably a homolog or functional equivalent as depicted in column 8 of Table II B, application no. 6, and being depicted in the same respective line as said B1597, and preferably the activity is increased non-targeted, whereby the respective line discloses in column 7 the fine chemical isoleucine.

Accordingly, in one embodiment, the molecule which activity is to be increased in the process of the invention is the gene product with an activity as a “acid shock protein”, preferably it is the molecule of section (a) or (b) of this paragraph.

In particular, it was observed that in plants, especially in Arabidopsis thaliana, increasing or generating the activity of a gene product non-targeted with the activity of a “acid shock protein”, preferably being encoded by a gene comprising the nucleic acid sequence SEQ ID NO. 38300, preferably the coding region thereof, conferred the production of or the increase in isoleucine compared with the wild type control.

The nucleic acid sequence of B1597 from Escherichia coli, e.g. as shown in the respective line in column 5 of Table I, application no. 6, has been published in B. And the activity of the gene product thereof is the activity of acid shock protein.

Accordingly, in one embodiment, the process of the present invention for producing leucine in a non-human organism, like a microorganism or a plant or a part thereof, comprises increasing or generating the activity of a gene product with the activity of a gene product conferring the activity of “acid shock protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B1597, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1597, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1597, or a functional equivalent or a homolog thereof as depicted in column 8 of Table II, application no. 6, preferably a homolog or functional equivalent as depicted in column 8 of Table II B, application no. 6, and being depicted in the same respective line as said B1597, and preferably the activity is increased non-targeted, whereby the respective line discloses in column 7 the fine chemical leucine.

Accordingly, in one embodiment, the molecule which activity is to be increased in the process of the invention is the gene product with an activity as a “acid shock protein”, preferably it is the molecule of section (a) or (b) of this paragraph.

In particular, it was observed that in plants, especially in Arabidopsis thaliana, increasing or generating the activity of a gene product non-targeted with the activity of a “acid shock protein”, preferably being encoded by a gene comprising the nucleic acid sequence SEQ ID NO. 38300, preferably the coding region thereof, conferred the production of or the increase in leucine compared with the wild type control.

The nucleic acid sequence of B1597 from Escherichia coli, e.g. as shown in the respective line in column 5 of Table I, application no. 6, has been published in B. And the activity of the gene product thereof is the activity of acid shock protein.

Accordingly, in one embodiment, the process of the present invention for producing valine in a non-human organism, like a microorganism or a plant or a part thereof, comprises increasing or generating the activity of a gene product with the activity of a gene product conferring the activity of “acid shock protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B1597, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1597, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1597, or a functional equivalent or a homolog thereof as depicted in column 8 of Table II, application no. 6, preferably a homolog or functional equivalent as depicted in column 8 of Table II B, application no. 6, and being depicted in the same respective line as said B1597, and preferably the activity is increased non-targeted, whereby the respective line discloses in column 7 the fine chemical valine.

Accordingly, in one embodiment, the molecule which activity is to be increased in the process of the invention is the gene product with an activity as a “acid shock protein”, preferably it is the molecule of section (a) or (b) of this paragraph.

In particular, it was observed that in plants, especially in Arabidopsis thaliana, increasing or generating the activity of a gene product non-targeted with the activity of a “acid shock protein”, preferably being encoded by a gene comprising the nucleic acid sequence SEQ ID NO. 38300, preferably the coding region thereof, conferred the production of or the increase in valine compared with the wild type control.

The nucleic acid sequence of B1601 from Escherichia coli, e.g. as shown in the respective line in column 5 of Table I, application no. 6, has been published in B. And the activity of the gene product thereof is the activity of transport protein.

Accordingly, in one embodiment, the process of the present invention for producing valine in a non-human organism, like a microorganism or a plant or a part thereof, comprises increasing or generating the activity of a gene product with the activity of a gene product conferring the activity of “transport protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B1601, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1601, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1601, or a functional equivalent or a homolog thereof as depicted in column 8 of Table II, application no. 6, preferably a homolog or functional equivalent as depicted in column 8 of Table II B, application no. 6, and being depicted in the same respective line as said B1601, and preferably the activity is increased non-targeted, whereby the respective line discloses in column 7 the fine chemical valine.

Accordingly, in one embodiment, the molecule which activity is to be increased in the process of the invention is the gene product with an activity as a “transport protein”, preferably it is the molecule of section (a) or (b) of this paragraph.

In particular, it was observed that in plants, especially in Arabidopsis thaliana, increasing or generating the activity of a gene product non-targeted with the activity of a “transport protein”, preferably being encoded by a gene comprising the nucleic acid sequence SEQ ID NO. 7992, preferably the coding region thereof, conferred the production of or the increase in valine compared with the wild type control.

The nucleic acid sequence of B1670 from Escherichia coli, e.g. as shown in the respective line in column 5 of Table I, application no. 6, has been published in B. And the activity of the gene product thereof is the activity of b1670-protein.

Accordingly, in one embodiment, the process of the present invention for producing valine in a non-human organism, like a microorganism or a plant or a part thereof, comprises increasing or generating the activity of a gene product with the activity of a gene product conferring the activity of “b1670-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B1670, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1670, or a functional equivalent or a homolog thereof as depicted in column 8 of Table II, application no. 6, preferably a homolog or functional equivalent as depicted in column 8 of Table II B, application no. 6, and being depicted in the same respective line as said B1670, and preferably the activity is increased non-targeted, whereby the respective line discloses in column 7 the fine chemical valine.

Accordingly, in one embodiment, the molecule which activity is to be increased in the process of the invention is the gene product with an activity as a “b1670-protein”, preferably it is the molecule of section (a) or (b) of this paragraph.

In particular, it was observed that in plants, especially in Arabidopsis thaliana, increasing or generating the activity of a gene product non-targeted with the activity of a “b1670-protein”, preferably being encoded by a gene comprising the nucleic acid sequence SEQ ID NO. 78753, preferably the coding region thereof, conferred the production of or the increase in valine compared with the wild type control.

The nucleic acid sequence of B1672 from Escherichia coli, e.g. as shown in the respective line in column 5 of Table I, application no. 6, has been published in B. And the activity of the gene product thereof is the activity of b1672-protein.

Accordingly, in one embodiment, the process of the present invention for producing valine in a non-human organism, like a microorganism or a plant or a part thereof, comprises increasing or generating the activity of a gene product with the activity of a gene product conferring the activity of “b1672-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B1672, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1672, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1672, or a functional equivalent or a homolog thereof as depicted in column 8 of Table II, application no. 6, preferably a homolog or functional equivalent as depicted in column 8 of Table II B, application no. 6, and being depicted in the same respective line as said B1672, and preferably the activity is increased non-targeted, whereby the respective line discloses in column 7 the fine chemical valine.

Accordingly, in one embodiment, the molecule which activity is to be increased in the process of the invention is the gene product with an activity as a “b1672-protein”, preferably it is the molecule of section (a) or (b) of this paragraph.

In particular, it was observed that in plants, especially in Arabidopsis thaliana, increasing or generating the activity of a gene product non-targeted with the activity of a “b1672-protein”, preferably being encoded by a gene comprising the nucleic acid sequence SEQ ID NO. 78771, preferably the coding region thereof, conferred the production of or the increase in valine compared with the wild type control.

The nucleic acid sequence of B1854 from Escherichia coli, e.g. as shown in the respective line in column 5 of Table I, application no. 6, has been published in B. And the activity of the gene product thereof is the activity of pyruvate kinase.

Accordingly, in one embodiment, the process of the present invention for producing leucine in a non-human organism, like a microorganism or a plant or a part thereof, comprises increasing or generating the activity of a gene product with the activity of a gene product conferring the activity of “pyruvate kinase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B1854, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1854, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1854, or a functional equivalent or a homolog thereof as depicted in column 8 of Table II, application no. 6, preferably a homolog or functional equivalent as depicted in column 8 of Table II B, application no. 6, and being depicted in the same respective line as said B1854, and preferably the activity is increased plastidic, whereby the respective line discloses in column 7 the fine chemical leucine.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 8363, preferably the coding region thereof, conferred the production of or the increase in leucine compared with the wild type control.

The nucleic acid sequence of B1873 from Escherichia coli, e.g. as shown in the respective line in column 5 of Table I, application no. 6, has been published in B. And the activity of the gene product thereof is the activity of cytochrome c-type protein.

Accordingly, in one embodiment, the process of the present invention for producing valine in a non-human organism, like a microorganism or a plant or a part thereof, comprises increasing or generating the activity of a gene product with the activity of a gene product conferring the activity of “cytochrome c-type protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B1873, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1873, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1873, or a functional equivalent or a homolog thereof as depicted in column 8 of Table II, application no. 6, preferably a homolog or functional equivalent as depicted in column 8 of Table II B, application no. 6, and being depicted in the same respective line as said B1873, and preferably the activity is increased non-targeted, whereby the respective line discloses in column 7 the fine chemical valine.

Accordingly, in one embodiment, the molecule which activity is to be increased in the process of the invention is the gene product with an activity as a “cytochrome c-type protein”, preferably it is the molecule of section (a) or (b) of this paragraph.

In particular, it was observed that in plants, especially in Arabidopsis thaliana, increasing or generating the activity of a gene product non-targeted with the activity of a “cytochrome c-type protein”, preferably being encoded by a gene comprising the nucleic acid sequence SEQ ID NO. 78970, preferably the coding region thereof, conferred the production of or the increase in valine compared with the wild type control.

The nucleic acid sequence of B1897 from Escherichia coli, e.g. as shown in the respective line in column 5 of Table I, application no. 6, has been published in B. And the activity of the gene product thereof is the activity of trehalose-phosphatase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “trehalose-phosphatase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B1897, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1897, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1897, or a functional equivalent or a homolog thereof as depicted in column 8 of Table II, application no. 6, preferably a homolog or functional equivalent as depicted in column 8 of Table II B, application no. 6, and being depicted in the same respective line as said B1897, and preferably the activity is increased non-targeted, whereby the respective line discloses in column 7 the fine chemical isoleucine.

Accordingly, in one embodiment, the molecule which activity is to be increased in the process of the invention is the gene product with an activity as a “trehalose-phosphatase”, preferably it is the molecule of section (a) or (b) of this paragraph.

In particular, it was observed that in plants, especially in Arabidopsis thaliana, increasing or generating the activity of a gene product non-targeted with the activity of a “trehalosephosphatase”, preferably being encoded by a gene comprising the nucleic acid sequence SEQ ID NO. 79056, preferably the coding region thereof, conferred the production of or the increase in isoleucine compared with the wild type control.

The nucleic acid sequence of B1897 from Escherichia coli, e.g. as shown in the respective line in column 5 of Table I, application no. 6, has been published in B. And the activity of the gene product thereof is the activity of trehalose-phosphatase.

Accordingly, in one embodiment, the process of the present invention for producing leucine in a non-human organism, like a microorganism or a plant or a part thereof, comprises increasing or generating the activity of a gene product with the activity of a gene product conferring the activity of “trehalose-phosphatase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B1897, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1897, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1897, or a functional equivalent or a homolog thereof as depicted in column 8 of Table II, application no. 6, preferably a homolog or functional equivalent as depicted in column 8 of Table II B, application no. 6, and being depicted in the same respective line as said B1897, and preferably the activity is increased non-targeted, whereby the respective line discloses in column 7 the fine chemical leucine.

Accordingly, in one embodiment, the molecule which activity is to be increased in the process of the invention is the gene product with an activity as a “trehalose-phosphatase”, preferably it is the molecule of section (a) or (b) of this paragraph.

In particular, it was observed that in plants, especially in Arabidopsis thaliana, increasing or generating the activity of a gene product non-targeted with the activity of a “trehalosephosphatase”, preferably being encoded by a gene comprising the nucleic acid sequence SEQ ID NO. 79056, preferably the coding region thereof, conferred the production of or the increase in leucine compared with the wild type control.

The nucleic acid sequence of B1897 from Escherichia coli, e.g. as shown in the respective line in column 5 of Table I, application no. 6, has been published in B. And the activity of the gene product thereof is the activity of trehalose-phosphatase.

Accordingly, in one embodiment, the process of the present invention for producing valine in a non-human organism, like a microorganism or a plant or a part thereof, comprises increasing or generating the activity of a gene product with the activity of a gene product conferring the activity of “trehalose-phosphatase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B1897, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B1897, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B1897, or a functional equivalent or a homolog thereof as depicted in column 8 of Table II, application no. 6, preferably a homolog or functional equivalent as depicted in column 8 of Table II B, application no. 6, and being depicted in the same respective line as said B1897, and preferably the activity is increased non-targeted, whereby the respective line discloses in column 7 the fine chemical valine.

Accordingly, in one embodiment, the molecule which activity is to be increased in the process of the invention is the gene product with an activity as a “trehalose-phosphatase”, preferably it is the molecule of section (a) or (b) of this paragraph.

In particular, it was observed that in plants, especially in Arabidopsis thaliana, increasing or generating the activity of a gene product non-targeted with the activity of a “trehalosephosphatase”, preferably being encoded by a gene comprising the nucleic acid sequence SEQ ID NO. 79056, preferably the coding region thereof, conferred the production of or the increase in valine compared with the wild type control.

The nucleic acid sequence of B2027 from Escherichia coli, e.g. as shown in the respective line in column 5 of Table I, application no. 6, has been published in B. And the activity of the gene product thereof is the activity of O-antigen chain length determinant.

Accordingly, in one embodiment, the process of the present invention for producing isoleucine in a non-human organism, like a microorganism or a plant or a part thereof, comprises increasing or generating the activity of a gene product with the activity of a gene product conferring the activity of “O-antigen chain length determinant”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B2027, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2027, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2027, or a functional equivalent or a homolog thereof as depicted in column 8 of Table II, application no. 6, preferably a homolog or functional equivalent as depicted in column 8 of Table II B, application no. 6, and being depicted in the same respective line as said B2027, and preferably the activity is increased non-targeted, whereby the respective line discloses in column 7 the fine chemical isoleucine.

Accordingly, in one embodiment, the molecule which activity is to be increased in the process of the invention is the gene product with an activity as a “O-antigen chain length determinant”, preferably it is the molecule of section (a) or (b) of this paragraph.

In particular, it was observed that in plants, especially in Arabidopsis thaliana, increasing or generating the activity of a gene product non-targeted with the activity of a “O-antigen chain length determinant”, preferably being encoded by a gene comprising the nucleic acid sequence SEQ ID NO. 79217, preferably the coding region thereof, conferred the production of or the increase in isoleucine compared with the wild type control.

The nucleic acid sequence of B2027 from Escherichia coli, e.g. as shown in the respective line in column 5 of Table I, application no. 6, has been published in B. And the activity of the gene product thereof is the activity of O-antigen chain length determinant.

Accordingly, in one embodiment, the process of the present invention for producing leucine in a non-human organism, like a microorganism or a plant or a part thereof, comprises increasing or generating the activity of a gene product with the activity of a gene product conferring the activity of “O-antigen chain length determinant”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B2027, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2027, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2027, or a functional equivalent or a homolog thereof as depicted in column 8 of Table II, application no. 6, preferably a homolog or functional equivalent as depicted in column 8 of Table II B, application no. 6, and being depicted in the same respective line as said B2027, and preferably the activity is increased non-targeted, whereby the respective line discloses in column 7 the fine chemical leucine.

Accordingly, in one embodiment, the molecule which activity is to be increased in the process of the invention is the gene product with an activity as a “O-antigen chain length determinant”, preferably it is the molecule of section (a) or (b) of this paragraph.

In particular, it was observed that in plants, especially in Arabidopsis thaliana, increasing or generating the activity of a gene product non-targeted with the activity of a “O-antigen chain length determinant”, preferably being encoded by a gene comprising the nucleic acid sequence SEQ ID NO. 79217, preferably the coding region thereof, conferred the production of or the increase in leucine compared with the wild type control.

The nucleic acid sequence of B2053 from Escherichia coli, e.g. as shown in the respective line in column 5 of Table I, application no. 6, has been published in B. And the activity of the gene product thereof is the activity of GDP-mannose 4,6-dehydratase.

Accordingly, in one embodiment, the process of the present invention for producing valine in a non-human organism, like a microorganism or a plant or a part thereof, comprises increasing or generating the activity of a gene product with the activity of a gene product conferring the activity of “GDP-mannose 4,6-dehydratase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B2053, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2053, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2053, or a functional equivalent or a homolog thereof as depicted in column 8 of Table II, application no. 6, preferably a homolog or functional equivalent as depicted in column 8 of Table II B, application no. 6, and being depicted in the same respective line as said B2053, and preferably the activity is increased non-targeted, whereby the respective line discloses in column 7 the fine chemical valine.

Accordingly, in one embodiment, the molecule which activity is to be increased in the process of the invention is the gene product with an activity as a “GDP-mannose 4,6-dehydratase”, preferably it is the molecule of section (a) or (b) of this paragraph.

In particular, it was observed that in plants, especially in Arabidopsis thaliana, increasing or generating the activity of a gene product non-targeted with the activity of a “GDP-mannose 4,6-dehydratase”, preferably being encoded by a gene comprising the nucleic acid sequence SEQ ID NO. 104277, preferably the coding region thereof, conferred the production of or the increase in valine compared with the wild type control.

The nucleic acid sequence of B2063 from Escherichia coli, e.g. as shown in the respective line in column 5 of Table I, application no. 6, has been published in B. And the activity of the gene product thereof is the activity of transport protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “transport protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B2063, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2063, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2063, or a functional equivalent or a homolog thereof as depicted in column 8 of Table II, application no. 6, preferably a homolog or functional equivalent as depicted in column 8 of Table II B, application no. 6, and being depicted in the same respective line as said B2063, and preferably the activity is increased non-targeted, whereby the respective line discloses in column 7 the fine chemical isoleucine.

Accordingly, in one embodiment, the molecule which activity is to be increased in the process of the invention is the gene product with an activity as a “transport protein”, preferably it is the molecule of section (a) or (b) of this paragraph.

In particular, it was observed that in plants, especially in Arabidopsis thaliana, increasing or generating the activity of a gene product non-targeted with the activity of a “transport protein”, preferably being encoded by a gene comprising the nucleic acid sequence SEQ ID NO. 38947, preferably the coding region thereof, conferred the production of or the increase in isoleucine compared with the wild type control.

The nucleic acid sequence of B2063 from Escherichia coli, e.g. as shown in the respective line in column 5 of Table I, application no. 6, has been published in B. And the activity of the gene product thereof is the activity of transport protein.

Accordingly, in one embodiment, the process of the present invention for producing leucine in a non-human organism, like a microorganism or a plant or a part thereof, comprises increasing or generating the activity of a gene product with the activity of a gene product conferring the activity of “transport protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B2063, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2063, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2063, or a functional equivalent or a homolog thereof as depicted in column 8 of Table II, application no. 6, preferably a homolog or functional equivalent as depicted in column 8 of Table II B, application no. 6, and being depicted in the same respective line as said B2063, and preferably the activity is increased non-targeted, whereby the respective line discloses in column 7 the fine chemical leucine.

Accordingly, in one embodiment, the molecule which activity is to be increased in the process of the invention is the gene product with an activity as a “transport protein”, preferably it is the molecule of section (a) or (b) of this paragraph.

In particular, it was observed that in plants, especially in Arabidopsis thaliana, increasing or generating the activity of a gene product non-targeted with the activity of a “transport protein”, preferably being encoded by a gene comprising the nucleic acid sequence SEQ ID NO. 38947, preferably the coding region thereof, conferred the production of or the increase in leucine compared with the wild type control.

The nucleic acid sequence of B2291 from Escherichia coli, e.g. as shown in the respective line in column 5 of Table I, application no. 6, has been published in B. And the activity of the gene product thereof is the activity of Nucleoside monophosphate phosphohydrolase.

Accordingly, in one embodiment, the process of the present invention for producing valine in a non-human organism, like a microorganism or a plant or a part thereof, comprises increasing or generating the activity of a gene product with the activity of a gene product conferring the activity of “Nucleoside monophosphate phosphohydrolase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B2291, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2291, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2291, or a functional equivalent or a homolog thereof as depicted in column 8 of Table II, application no. 6, preferably a homolog or functional equivalent as depicted in column 8 of Table II B, application no. 6, and being depicted in the same respective line as said B2291, and preferably the activity is increased non-targeted, whereby the respective line discloses in column 7 the fine chemical valine.

Accordingly, in one embodiment, the molecule which activity is to be increased in the process of the invention is the gene product with an activity as a “Nucleoside monophosphate phosphohydrolase”, preferably it is the molecule of section (a) or (b) of this paragraph.

In particular, it was observed that in plants, especially in Arabidopsis thaliana, increasing or generating the activity of a gene product non-targeted with the activity of a “Nucleoside monophosphate phosphohydrolase”, preferably being encoded by a gene comprising the nucleic acid sequence SEQ ID NO. 104616, preferably the coding region thereof, conferred the production of or the increase in valine compared with the wild type control.

The nucleic acid sequence of B2405 from Escherichia coli, e.g. as shown in the respective line in column 5 of Table I, application no. 6, has been published in B. And the activity of the gene product thereof is the activity of transcriptional regulator.

Accordingly, in one embodiment, the process of the present invention for producing isoleucine in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B2405, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2405, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2405, or a functional equivalent or a homolog thereof as depicted in column 8 of Table II, application no. 6, preferably a homolog or functional equivalent as depicted in column 8 of Table II B, application no. 6, and being depicted in the same respective line as said B2405, and preferably the activity is increased non-targeted, whereby the respective line discloses in column 7 the fine chemical isoleucine.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 39255, preferably the coding region thereof, conferred the production of or the increase in isoleucine compared with the wild type control.

The nucleic acid sequence of B2405 from Escherichia coli, e.g. as shown in the respective line in column 5 of Table I, application no. 6, has been published in B. And the activity of the gene product thereof is the activity of transcriptional regulator.

Accordingly, in one embodiment, the process of the present invention for producing valine in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B2405, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2405, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2405, or a functional equivalent or a homolog thereof as depicted in column 8 of Table II, application no. 6, preferably a homolog or functional equivalent as depicted in column 8 of Table II B, application no. 6, and being depicted in the same respective line as said B2405, and preferably the activity is increased non-targeted, whereby the respective line discloses in column 7 the fine chemical valine.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 39255, preferably the coding region thereof, conferred the production of or the increase in valine compared with the wild type control.

The nucleic acid sequence of B2513 from Escherichia coli, e.g. as shown in the respective line in column 5 of Table I, application no. 6, has been published in B. And the activity of the gene product thereof is the activity of b2513-protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “b2513-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B2513, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2513, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2513, or a functional equivalent or a homolog thereof as depicted in column 8 of Table II, application no. 6, preferably a homolog or functional equivalent as depicted in column 8 of Table II B, application no. 6, and being depicted in the same respective line as said B2513, and preferably the activity is increased non-targeted, whereby the respective line discloses in column 7 the fine chemical isoleucine.

Accordingly, in one embodiment, the molecule which activity is to be increased in the process of the invention is the gene product with an activity as a “b2513-protein”, preferably it is the molecule of section (a) or (b) of this paragraph.

In particular, it was observed that in plants, especially in Arabidopsis thaliana, increasing or generating the activity of a gene product non-targeted with the activity of a “b2513-protein”, preferably being encoded by a gene comprising the nucleic acid sequence SEQ ID NO. 9167, preferably the coding region thereof, conferred the production of or the increase in isoleucine compared with the wild type control.

The nucleic acid sequence of B2513 from Escherichia coli, e.g. as shown in the respective line in column 5 of Table I, application no. 6, has been published in B. And the activity of the gene product thereof is the activity of b2513-protein.

Accordingly, in one embodiment, the process of the present invention for producing leucine in a non-human organism, like a microorganism or a plant or a part thereof, comprises increasing or generating the activity of a gene product with the activity of a gene product conferring the activity of “b2513-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B2513, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2513, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2513, or a functional equivalent or a homolog thereof as depicted in column 8 of Table II, application no. 6, preferably a homolog or functional equivalent as depicted in column 8 of Table II B, application no. 6, and being depicted in the same respective line as said B2513, and preferably the activity is increased non-targeted, whereby the respective line discloses in column 7 the fine chemical leucine.

Accordingly, in one embodiment, the molecule which activity is to be increased in the process of the invention is the gene product with an activity as a “b2513-protein”, preferably it is the molecule of section (a) or (b) of this paragraph.

In particular, it was observed that in plants, especially in Arabidopsis thaliana, increasing or generating the activity of a gene product non-targeted with the activity of a “b2513-protein”, preferably being encoded by a gene comprising the nucleic acid sequence SEQ ID NO. 9167, preferably the coding region thereof, conferred the production of or the increase in leucine compared with the wild type control.

The nucleic acid sequence of B2513 from Escherichia coli, e.g. as shown in the respective line in column 5 of Table I, application no. 6, has been published in B. And the activity of the gene product thereof is the activity of b2513-protein.

Accordingly, in one embodiment, the process of the present invention for producing valine in a non-human organism, like a microorganism or a plant or a part thereof, comprises increasing or generating the activity of a gene product with the activity of a gene product conferring the activity of “b2513-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B2513, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2513, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2513, or a functional equivalent or a homolog thereof as depicted in column 8 of Table II, application no. 6, preferably a homolog or functional equivalent as depicted in column 8 of Table II B, application no. 6, and being depicted in the same respective line as said B2513, and preferably the activity is increased non-targeted, whereby the respective line discloses in column 7 the fine chemical valine.

Accordingly, in one embodiment, the molecule which activity is to be increased in the process of the invention is the gene product with an activity as a “b2513-protein”, preferably it is the molecule of section (a) or (b) of this paragraph.

In particular, it was observed that in plants, especially in Arabidopsis thaliana, increasing or generating the activity of a gene product non-targeted with the activity of a “b2513-protein”, preferably being encoded by a gene comprising the nucleic acid sequence SEQ ID NO. 9167, preferably the coding region thereof, conferred the production of or the increase in valine compared with the wild type control.

The nucleic acid sequence of B2551 from Escherichia coli, e.g. as shown in the respective line in column 5 of Table I, application no. 6, has been published in B. And the activity of the gene product thereof is the activity of serine hydroxymethyltransferase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “serine hydroxymethyltransferase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B2551, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2551, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2551, or a functional equivalent or a homolog thereof as depicted in column 8 of Table II, application no. 6, preferably a homolog or functional equivalent as depicted in column 8 of Table II B, application no. 6, and being depicted in the same respective line as said B2551, and preferably the activity is increased non-targeted, whereby the respective line discloses in column 7 the fine chemical isoleucine.

Accordingly, in one embodiment, the molecule which activity is to be increased in the process of the invention is the gene product with an activity as a “serine hydroxymethyltransferase”, preferably it is the molecule of section (a) or (b) of this paragraph.

In particular, it was observed that in plants, especially in Arabidopsis thaliana, increasing or generating the activity of a gene product non-targeted with the activity of a “serine hydroxymethyltransferase”, preferably being encoded by a gene comprising the nucleic acid sequence SEQ ID NO. 79627, preferably the coding region thereof, conferred the production of or the increase in isoleucine compared with the wild type control.

The nucleic acid sequence of B2613 from Escherichia coli, e.g. as shown in the respective line in column 5 of Table I, application no. 6, has been published in B. And the activity of the gene product thereof is the activity of b2613-protein.

Accordingly, in one embodiment, the process of the present invention for producing valine in a non-human organism, like a microorganism or a plant or a part thereof, comprises increasing or generating the activity of a gene product with the activity of a gene product conferring the activity of “b2613-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B2613, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2613, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2613, or a functional equivalent or a homolog thereof as depicted in column 8 of Table II, application no. 6, preferably a homolog or functional equivalent as depicted in column 8 of Table II B, application no. 6, and being depicted in the same respective line as said B2613, and preferably the activity is increased non-targeted, whereby the respective line discloses in column 7 the fine chemical valine.

Accordingly, in one embodiment, the molecule which activity is to be increased in the process of the invention is the gene product with an activity as a “b2613-protein”, preferably it is the molecule of section (a) or (b) of this paragraph.

In particular, it was observed that in plants, especially in Arabidopsis thaliana, increasing or generating the activity of a gene product non-targeted with the activity of a “b2613-protein”, preferably being encoded by a gene comprising the nucleic acid sequence SEQ ID NO. 40665, preferably the coding region thereof, conferred the production of or the increase in valine compared with the wild type control.

The nucleic acid sequence of B2634 from Escherichia coli, e.g. as shown in the respective line in column 5 of Table I, application no. 6, has been published in B. And the activity of the gene product thereof is the activity of transcriptional regulator.

Accordingly, in one embodiment, the process of the present invention for producing isoleucine in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B2634, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2634, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2634, or a functional equivalent or a homolog thereof as depicted in column 8 of Table II, application no. 6, preferably a homolog or functional equivalent as depicted in column 8 of Table II B, application no. 6, and being depicted in the same respective line as said B2634, and preferably the activity is increased non-targeted, whereby the respective line discloses in column 7 the fine chemical isoleucine.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 40726, preferably the coding region thereof, conferred the production of or the increase in isoleucine compared with the wild type control.

The nucleic acid sequence of B2634 from Escherichia coli, e.g. as shown in the respective line in column 5 of Table I, application no. 6, has been published in B. And the activity of the gene product thereof is the activity of transcriptional regulator.

Accordingly, in one embodiment, the process of the present invention for producing leucine in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B2634, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2634, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2634, or a functional equivalent or a homolog thereof as depicted in column 8 of Table II, application no. 6, preferably a homolog or functional equivalent as depicted in column 8 of Table II B, application no. 6, and being depicted in the same respective line as said B2634, and preferably the activity is increased non-targeted, whereby the respective line discloses in column 7 the fine chemical leucine.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 40726, preferably the coding region thereof, conferred the production of or the increase in leucine compared with the wild type control.

The nucleic acid sequence of B2673 from Escherichia coli, e.g. as shown in the respective line in column 5 of Table I, application no. 6, has been published in B. And the activity of the gene product thereof is the activity of b2673-protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “b2673-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B2673, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2673, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2673, or a functional equivalent or a homolog thereof as depicted in column 8 of Table II, application no. 6, preferably a homolog or functional equivalent as depicted in column 8 of Table II B, application no. 6, and being depicted in the same respective line as said B2673, and preferably the activity is increased non-targeted, whereby the respective line discloses in column 7 the fine chemical isoleucine.

Accordingly, in one embodiment, the molecule which activity is to be increased in the process of the invention is the gene product with an activity as a “b2673-protein”, preferably it is the molecule of section (a) or (b) of this paragraph.

In particular, it was observed that in plants, especially in Arabidopsis thaliana, increasing or generating the activity of a gene product non-targeted with the activity of a “b2673-protein”, preferably being encoded by a gene comprising the nucleic acid sequence SEQ ID NO. 9244, preferably the coding region thereof, conferred the production of or the increase in isoleucine compared with the wild type control.

The nucleic acid sequence of B2701 from Escherichia coli, e.g. as shown in the respective line in column 5 of Table I, application no. 6, has been published in B. And the activity of the gene product thereof is the activity of murein transglycosylase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “murein transglycosylase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B2701, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2701, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2701, or a functional equivalent or a homolog thereof as depicted in column 8 of Table II, application no. 6, preferably a homolog or functional equivalent as depicted in column 8 of Table II B, application no. 6, and being depicted in the same respective line as said B2701, and preferably the activity is increased non-targeted, whereby the respective line discloses in column 7 the fine chemical isoleucine.

Accordingly, in one embodiment, the molecule which activity is to be increased in the process of the invention is the gene product with an activity as a “murein transglycosylase”, preferably it is the molecule of section (a) or (b) of this paragraph.

In particular, it was observed that in plants, especially in Arabidopsis thaliana, increasing or generating the activity of a gene product non-targeted with the activity of a “murein transglycosylase”, preferably being encoded by a gene comprising the nucleic acid sequence SEQ ID NO. 80756, preferably the coding region thereof, conferred the production of or the increase in isoleucine compared with the wild type control.

The nucleic acid sequence of B2701 from Escherichia coli, e.g. as shown in the respective line in column 5 of Table I, application no. 6, has been published in B. And the activity of the gene product thereof is the activity of murein transglycosylase.

Accordingly, in one embodiment, the process of the present invention for producing leucine in a non-human organism, like a microorganism or a plant or a part thereof, comprises increasing or generating the activity of a gene product with the activity of a gene product conferring the activity of “murein transglycosylase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B2701, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2701, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2701, or a functional equivalent or a homolog thereof as depicted in column 8 of Table II, application no. 6, preferably a homolog or functional equivalent as depicted in column 8 of Table II B, application no. 6, and being depicted in the same respective line as said B2701, and preferably the activity is increased non-targeted, whereby the respective line discloses in column 7 the fine chemical leucine.

Accordingly, in one embodiment, the molecule which activity is to be increased in the process of the invention is the gene product with an activity as a “murein transglycosylase”, preferably it is the molecule of section (a) or (b) of this paragraph.

In particular, it was observed that in plants, especially in Arabidopsis thaliana, increasing or generating the activity of a gene product non-targeted with the activity of a “murein transglycosylase”, preferably being encoded by a gene comprising the nucleic acid sequence SEQ ID NO. 80756, preferably the coding region thereof, conferred the production of or the increase in leucine compared with the wild type control.

The nucleic acid sequence of B2701 from Escherichia coli, e.g. as shown in the respective line in column 5 of Table I, application no. 6, has been published in B. And the activity of the gene product thereof is the activity of murein transglycosylase.

Accordingly, in one embodiment, the process of the present invention for producing valine in a non-human organism, like a microorganism or a plant or a part thereof, comprises increasing or generating the activity of a gene product with the activity of a gene product conferring the activity of “murein transglycosylase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B2701, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2701, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2701, or a functional equivalent or a homolog thereof as depicted in column 8 of Table II, application no. 6, preferably a homolog or functional equivalent as depicted in column 8 of Table II B, application no. 6, and being depicted in the same respective line as said B2701, and preferably the activity is increased non-targeted, whereby the respective line discloses in column 7 the fine chemical valine.

Accordingly, in one embodiment, the molecule which activity is to be increased in the process of the invention is the gene product with an activity as a “murein transglycosylase”, preferably it is the molecule of section (a) or (b) of this paragraph.

In particular, it was observed that in plants, especially in Arabidopsis thaliana, increasing or generating the activity of a gene product non-targeted with the activity of a “murein transglycosylase”, preferably being encoded by a gene comprising the nucleic acid sequence SEQ ID NO. 80756, preferably the coding region thereof, conferred the production of or the increase in valine compared with the wild type control.

The nucleic acid sequence of B2714 from Escherichia coli, e.g. as shown in the respective line in column 5 of Table I, application no. 6, has been published in B. And the activity of the gene product thereof is the activity of transcriptional regulator.

Accordingly, in one embodiment, the process of the present invention for producing valine in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B2714, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2714, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2714, or a functional equivalent or a homolog thereof as depicted in column 8 of Table II, application no. 6, preferably a homolog or functional equivalent as depicted in column 8 of Table II B, application no. 6, and being depicted in the same respective line as said B2714, and preferably the activity is increased non-targeted, whereby the respective line discloses in column 7 the fine chemical valine.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 40741, preferably the coding region thereof, conferred the production of or the increase in valine compared with the wild type control.

The nucleic acid sequence of B2739 from Escherichia coli, e.g. as shown in the respective line in column 5 of Table I, application no. 6, has been published in B. And the activity of the gene product thereof is the activity of b2739-protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “b2739-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B2739, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2739, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2739, or a functional equivalent or a homolog thereof as depicted in column 8 of Table II, application no. 6, preferably a homolog or functional equivalent as depicted in column 8 of Table II B, application no. 6, and being depicted in the same respective line as said B2739, and preferably the activity is increased non-targeted, whereby the respective line discloses in column 7 the fine chemical isoleucine.

Accordingly, in one embodiment, the molecule which activity is to be increased in the process of the invention is the gene product with an activity as a “b2739-protein”, preferably it is the molecule of section (a) or (b) of this paragraph.

In particular, it was observed that in plants, especially in Arabidopsis thaliana, increasing or generating the activity of a gene product non-targeted with the activity of a “b2739-protein”, preferably being encoded by a gene comprising the nucleic acid sequence SEQ ID NO. 80906, preferably the coding region thereof, conferred the production of or the increase in isoleucine compared with the wild type control.

The nucleic acid sequence of B2739 from Escherichia coli, e.g. as shown in the respective line in column 5 of Table I, application no. 6, has been published in B. And the activity of the gene product thereof is the activity of b2739-protein.

Accordingly, in one embodiment, the process of the present invention for producing leucine in a non-human organism, like a microorganism or a plant or a part thereof, comprises increasing or generating the activity of a gene product with the activity of a gene product conferring the activity of “b2739-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B2739, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2739, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2739, or a functional equivalent or a homolog thereof as depicted in column 8 of Table II, application no. 6, preferably a homolog or functional equivalent as depicted in column 8 of Table II B, application no. 6, and being depicted in the same respective line as said B2739, and preferably the activity is increased non-targeted, whereby the respective line discloses in column 7 the fine chemical leucine.

Accordingly, in one embodiment, the molecule which activity is to be increased in the process of the invention is the gene product with an activity as a “b2739-protein”, preferably it is the molecule of section (a) or (b) of this paragraph.

In particular, it was observed that in plants, especially in Arabidopsis thaliana, increasing or generating the activity of a gene product non-targeted with the activity of a “b2739-protein”, preferably being encoded by a gene comprising the nucleic acid sequence SEQ ID NO. 80906, preferably the coding region thereof, conferred the production of or the increase in leucine compared with the wild type control.

The nucleic acid sequence of B2739 from Escherichia coli, e.g. as shown in the respective line in column 5 of Table I, application no. 6, has been published in B. And the activity of the gene product thereof is the activity of b2739-protein.

Accordingly, in one embodiment, the process of the present invention for producing valine in a non-human organism, like a microorganism or a plant or a part thereof, comprises increasing or generating the activity of a gene product with the activity of a gene product conferring the activity of “b2739-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B2739, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2739, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2739, or a functional equivalent or a homolog thereof as depicted in column 8 of Table II, application no. 6, preferably a homolog or functional equivalent as depicted in column 8 of Table II B, application no. 6, and being depicted in the same respective line as said B2739, and preferably the activity is increased non-targeted, whereby the respective line discloses in column 7 the fine chemical valine.

Accordingly, in one embodiment, the molecule which activity is to be increased in the process of the invention is the gene product with an activity as a “b2739-protein”, preferably it is the molecule of section (a) or (b) of this paragraph.

In particular, it was observed that in plants, especially in Arabidopsis thaliana, increasing or generating the activity of a gene product non-targeted with the activity of a “b2739-protein”, preferably being encoded by a gene comprising the nucleic acid sequence SEQ ID NO. 80906, preferably the coding region thereof, conferred the production of or the increase in valine compared with the wild type control.

The nucleic acid sequence of B2812 from Escherichia coli, e.g. as shown in the respective line in column 5 of Table I, application no. 6, has been published in B. And the activity of the gene product thereof is the activity of b2812-protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “b2812-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B2812, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2812, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2812, or a functional equivalent or a homolog thereof as depicted in column 8 of Table II, application no. 6, preferably a homolog or functional equivalent as depicted in column 8 of Table II B, application no. 6, and being depicted in the same respective line as said B2812, and preferably the activity is increased non-targeted, whereby the respective line discloses in column 7 the fine chemical isoleucine.

Accordingly, in one embodiment, the molecule which activity is to be increased in the process of the invention is the gene product with an activity as a “b2812-protein”, preferably it is the molecule of section (a) or (b) of this paragraph.

In particular, it was observed that in plants, especially in Arabidopsis thaliana, increasing or generating the activity of a gene product non-targeted with the activity of a “b2812-protein”, preferably being encoded by a gene comprising the nucleic acid sequence SEQ ID NO. 40795, preferably the coding region thereof, conferred the production of or the increase in isoleucine compared with the wild type control.

The nucleic acid sequence of B2812 from Escherichia coli, e.g. as shown in the respective line in column 5 of Table I, application no. 6, has been published in B. And the activity of the gene product thereof is the activity of b2812-protein.

Accordingly, in one embodiment, the process of the present invention for producing leucine in a non-human organism, like a microorganism or a plant or a part thereof, comprises increasing or generating the activity of a gene product with the activity of a gene product conferring the activity of “b2812-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B2812, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2812, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2812, or a functional equivalent or a homolog thereof as depicted in column 8 of Table II, application no. 6, preferably a homolog or functional equivalent as depicted in column 8 of Table II B, application no. 6, and being depicted in the same respective line as said B2812, and preferably the activity is increased non-targeted, whereby the respective line discloses in column 7 the fine chemical leucine.

Accordingly, in one embodiment, the molecule which activity is to be increased in the process of the invention is the gene product with an activity as a “b2812-protein”, preferably it is the molecule of section (a) or (b) of this paragraph.

In particular, it was observed that in plants, especially in Arabidopsis thaliana, increasing or generating the activity of a gene product non-targeted with the activity of a “b2812-protein”, preferably being encoded by a gene comprising the nucleic acid sequence SEQ ID NO. 40795, preferably the coding region thereof, conferred the production of or the increase in leucine compared with the wild type control.

The nucleic acid sequence of B2839 from Escherichia coli, e.g. as shown in the respective line in column 5 of Table I, application no. 6, has been published in B. And the activity of the gene product thereof is the activity of transcriptional regulator.

Accordingly, in one embodiment, the process of the present invention for producing isoleucine in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B2839, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2839, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2839, or a functional equivalent or a homolog thereof as depicted in column 8 of Table II, application no. 6, preferably a homolog or functional equivalent as depicted in column 8 of Table II B, application no. 6, and being depicted in the same respective line as said B2839, and preferably the activity is increased non-targeted, whereby the respective line discloses in column 7 the fine chemical isoleucine.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 81935, preferably the coding region thereof, conferred the production of or the increase in isoleucine compared with the wild type control.

The nucleic acid sequence of B2839 from Escherichia coli, e.g. as shown in the respective line in column 5 of Table I, application no. 6, has been published in B. And the activity of the gene product thereof is the activity of transcriptional regulator.

Accordingly, in one embodiment, the process of the present invention for producing leucine in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B2839, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2839, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2839, or a functional equivalent or a homolog thereof as depicted in column 8 of Table II, application no. 6, preferably a homolog or functional equivalent as depicted in column 8 of Table II B, application no. 6, and being depicted in the same respective line as said B2839, and preferably the activity is increased non-targeted, whereby the respective line discloses in column 7 the fine chemical leucine.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 81935, preferably the coding region thereof, conferred the production of or the increase in leucine compared with the wild type control.

The nucleic acid sequence of B2839 from Escherichia coli, e.g. as shown in the respective line in column 5 of Table I, application no. 6, has been published in B. And the activity of the gene product thereof is the activity of transcriptional regulator.

Accordingly, in one embodiment, the process of the present invention for producing valine in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B2839, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2839, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2839, or a functional equivalent or a homolog thereof as depicted in column 8 of Table II, application no. 6, preferably a homolog or functional equivalent as depicted in column 8 of Table II B, application no. 6, and being depicted in the same respective line as said B2839, and preferably the activity is increased non-targeted, whereby the respective line discloses in column 7 the fine chemical valine.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 81935, preferably the coding region thereof, conferred the production of or the increase in valine compared with the wild type control.

The nucleic acid sequence of B2914 from Escherichia coli, e.g. as shown in the respective line in column 5 of Table I, application no. 6, has been published in B. And the activity of the gene product thereof is the activity of ribosephosphate isomerase, constitutive.

Accordingly, in one embodiment, the process of the present invention for producing isoleucine in a non-human organism, like a microorganism or a plant or a part thereof, comprises increasing or generating the activity of a gene product with the activity of a gene product conferring the activity of “ribosephosphate isomerase, constitutive”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B2914, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2914, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2914, or a functional equivalent or a homolog thereof as depicted in column 8 of Table II, application no. 6, preferably a homolog or functional equivalent as depicted in column 8 of Table II B, application no. 6, and being depicted in the same respective line as said B2914, and preferably the activity is increased non-targeted, whereby the respective line discloses in column 7 the fine chemical isoleucine.

Accordingly, in one embodiment, the molecule which activity is to be increased in the process of the invention is the gene product with an activity as a “ribosephosphate isomerase, constitutive”, preferably it is the molecule of section (a) or (b) of this paragraph.

In particular, it was observed that in plants, especially in Arabidopsis thaliana, increasing or generating the activity of a gene product non-targeted with the activity of a “ribosephosphate isomerase, constitutive”, preferably being encoded by a gene comprising the nucleic acid sequence SEQ ID NO. 41073, preferably the coding region thereof, conferred the production of or the increase in isoleucine compared with the wild type control.

The nucleic acid sequence of B2923 from Escherichia coli, e.g. as shown in the respective line in column 5 of Table I, application no. 6, has been published in B. And the activity of the gene product thereof is the activity of arginine exporter protein.

Accordingly, in one embodiment, the process of the present invention for producing valine in a non-human organism, like a microorganism or a plant or a part thereof, comprises increasing or generating the activity of a gene product with the activity of a gene product conferring the activity of “arginine exporter protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B2923, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B2923, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B2923, or a functional equivalent or a homolog thereof as depicted in column 8 of Table II, application no. 6, preferably a homolog or functional equivalent as depicted in column 8 of Table II B, application no. 6, and being depicted in the same respective line as said B2923, and preferably the activity is increased non-targeted, whereby the respective line discloses in column 7 the fine chemical valine.

Accordingly, in one embodiment, the molecule which activity is to be increased in the process of the invention is the gene product with an activity as a “arginine exporter protein”, preferably it is the molecule of section (a) or (b) of this paragraph.

In particular, it was observed that in plants, especially in Arabidopsis thaliana, increasing or generating the activity of a gene product non-targeted with the activity of a “arginine exporter protein”, preferably being encoded by a gene comprising the nucleic acid sequence SEQ ID NO. 9333, preferably the coding region thereof, conferred the production of or the increase in valine compared with the wild type control.

The nucleic acid sequence of B3064 from Escherichia coli, e.g. as shown in the respective line in column 5 of Table I, application no. 6, has been published in B. And the activity of the gene product thereof is the activity of glycoprotease.

Accordingly, in one embodiment, the process of the present invention for producing isoleucine in a non-human organism, like a microorganism or a plant or a part thereof, comprises increasing or generating the activity of a gene product with the activity of a gene product conferring the activity of “glycoprotease”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B3064, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3064, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3064, or a functional equivalent or a homolog thereof as depicted in column 8 of Table II, application no. 6, preferably a homolog or functional equivalent as depicted in column 8 of Table II B, application no. 6, and being depicted in the same respective line as said B3064, and preferably the activity is increased non-targeted, whereby the respective line discloses in column 7 the fine chemical isoleucine.

Accordingly, in one embodiment, the molecule which activity is to be increased in the process of the invention is the gene product with an activity as a “glycoprotease”, preferably it is the molecule of section (a) or (b) of this paragraph.

In particular, it was observed that in plants, especially in Arabidopsis thaliana, increasing or generating the activity of a gene product non-targeted with the activity of a “glycoprotease”, preferably being encoded by a gene comprising the nucleic acid sequence SEQ ID NO. 42046, preferably the coding region thereof, conferred the production of or the increase in isoleucine compared with the wild type control.

The nucleic acid sequence of B3064 from Escherichia coli, e.g. as shown in the respective line in column 5 of Table I, application no. 6, has been published in B. And the activity of the gene product thereof is the activity of glycoprotease.

Accordingly, in one embodiment, the process of the present invention for producing leucine in a non-human organism, like a microorganism or a plant or a part thereof, comprises increasing or generating the activity of a gene product with the activity of a gene product conferring the activity of “glycoprotease”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B3064, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3064, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3064, or a functional equivalent or a homolog thereof as depicted in column 8 of Table II, application no. 6, preferably a homolog or functional equivalent as depicted in column 8 of Table II B, application no. 6, and being depicted in the same respective line as said B3064, and preferably the activity is increased non-targeted, whereby the respective line discloses in column 7 the fine chemical leucine.

Accordingly, in one embodiment, the molecule which activity is to be increased in the process of the invention is the gene product with an activity as a “glycoprotease”, preferably it is the molecule of section (a) or (b) of this paragraph.

In particular, it was observed that in plants, especially in Arabidopsis thaliana, increasing or generating the activity of a gene product non-targeted with the activity of a “glycoprotease”, preferably being encoded by a gene comprising the nucleic acid sequence SEQ ID NO. 42046, preferably the coding region thereof, conferred the production of or the increase in leucine compared with the wild type control.

The nucleic acid sequence of B3064 from Escherichia coli, e.g. as shown in the respective line in column 5 of Table I, application no. 6, has been published in B. And the activity of the gene product thereof is the activity of glycoprotease.

Accordingly, in one embodiment, the process of the present invention for producing valine in a non-human organism, like a microorganism or a plant or a part thereof, comprises increasing or generating the activity of a gene product with the activity of a gene product conferring the activity of “glycoprotease”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B3064, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3064, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3064, or a functional equivalent or a homolog thereof as depicted in column 8 of Table II, application no. 6, preferably a homolog or functional equivalent as depicted in column 8 of Table II B, application no. 6, and being depicted in the same respective line as said B3064, and preferably the activity is increased non-targeted, whereby the respective line discloses in column 7 the fine chemical valine.

Accordingly, in one embodiment, the molecule which activity is to be increased in the process of the invention is the gene product with an activity as a “glycoprotease”, preferably it is the molecule of section (a) or (b) of this paragraph.

In particular, it was observed that in plants, especially in Arabidopsis thaliana, increasing or generating the activity of a gene product non-targeted with the activity of a “glycoprotease”, preferably being encoded by a gene comprising the nucleic acid sequence SEQ ID NO. 42046, preferably the coding region thereof, conferred the production of or the increase in valine compared with the wild type control.

The nucleic acid sequence of B3083 from Escherichia coli, e.g. as shown in the respective line in column 5 of Table I, application no. 6, has been published in B. And the activity of the gene product thereof is the activity of b3083-protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “b3083-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B3083, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3083, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3083, or a functional equivalent or a homolog thereof as depicted in column 8 of Table II, application no. 6, preferably a homolog or functional equivalent as depicted in column 8 of Table II B, application no. 6, and being depicted in the same respective line as said B3083, and preferably the activity is increased non-targeted, whereby the respective line discloses in column 7 the fine chemical isoleucine.

Accordingly, in one embodiment, the molecule which activity is to be increased in the process of the invention is the gene product with an activity as a “b3083-protein”, preferably it is the molecule of section (a) or (b) of this paragraph.

In particular, it was observed that in plants, especially in Arabidopsis thaliana, increasing or generating the activity of a gene product non-targeted with the activity of a “b3083-protein”, preferably being encoded by a gene comprising the nucleic acid sequence SEQ ID NO. 82424, preferably the coding region thereof, conferred the production of or the increase in isoleucine compared with the wild type control.

The nucleic acid sequence of B3083 from Escherichia coli, e.g. as shown in the respective line in column 5 of Table I, application no. 6, has been published in B. And the activity of the gene product thereof is the activity of b3083-protein.

Accordingly, in one embodiment, the process of the present invention for producing valine in a non-human organism, like a microorganism or a plant or a part thereof, comprises increasing or generating the activity of a gene product with the activity of a gene product conferring the activity of “b3083-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B3083, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3083, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3083, or a functional equivalent or a homolog thereof as depicted in column 8 of Table II, application no. 6, preferably a homolog or functional equivalent as depicted in column 8 of Table II B, application no. 6, and being depicted in the same respective line as said B3083, and preferably the activity is increased non-targeted, whereby the respective line discloses in column 7 the fine chemical valine.

Accordingly, in one embodiment, the molecule which activity is to be increased in the process of the invention is the gene product with an activity as a “b3083-protein”, preferably it is the molecule of section (a) or (b) of this paragraph.

In particular, it was observed that in plants, especially in Arabidopsis thaliana, increasing or generating the activity of a gene product non-targeted with the activity of a “b3083-protein”, preferably being encoded by a gene comprising the nucleic acid sequence SEQ ID NO. 82424, preferably the coding region thereof, conferred the production of or the increase in valine compared with the wild type control.

The nucleic acid sequence of B3112 from Escherichia coli, e.g. as shown in the respective line in column 5 of Table I, application no. 6, has been published in B. And the activity of the gene product thereof is the activity of L-serine dehydratase.

Accordingly, in one embodiment, the process of the present invention for producing leucine in a non-human organism, like a microorganism or a plant or a part thereof, comprises increasing or generating the activity of a gene product with the activity of a gene product conferring the activity of “L-serine dehydratase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B3112, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3112, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3112, or a functional equivalent or a homolog thereof as depicted in column 8 of Table II, application no. 6, preferably a homolog or functional equivalent as depicted in column 8 of Table II B, application no. 6, and being depicted in the same respective line as said B3112, and preferably the activity is increased plastidic, whereby the respective line discloses in column 7 the fine chemical leucine.

Accordingly, in one embodiment, the molecule which activity is to be increased in the process of the invention is the gene product with an activity as a “L-serine dehydratase”, preferably it is the molecule of section (a) or (b) of this paragraph.

In particular, it was observed that in plants, especially in Arabidopsis thaliana, increasing or generating the activity of a gene product plastidic with the activity of a “L-serine dehydratase”, preferably being encoded by a gene comprising the nucleic acid sequence SEQ ID NO. 104660, preferably the coding region thereof, conferred the production of or the increase in leucine compared with the wild type control.

The nucleic acid sequence of B3117 from Escherichia coli, e.g. as shown in the respective line in column 5 of Table I, application no. 6, has been published in B. And the activity of the gene product thereof is the activity of threonine dehydratase.

Accordingly, in one embodiment, the process of the present invention for producing valine in a non-human organism, like a microorganism or a plant or a part thereof, comprises increasing or generating the activity of a gene product with the activity of a gene product conferring the activity of “threonine dehydratase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B3117, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3117, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3117, or a functional equivalent or a homolog thereof as depicted in column 8 of Table II, application no. 6, preferably a homolog or functional equivalent as depicted in column 8 of Table II B, application no. 6, and being depicted in the same respective line as said B3117, and preferably the activity is increased plastidic, whereby the respective line discloses in column 7 the fine chemical valine.

Accordingly, in one embodiment, the molecule which activity is to be increased in the process of the invention is the gene product with an activity as a “threonine dehydratase”, preferably it is the molecule of section (a) or (b) of this paragraph.

In particular, it was observed that in plants, especially in Arabidopsis thaliana, increasing or generating the activity of a gene product plastidic with the activity of a “threonine dehydratase”, preferably being encoded by a gene comprising the nucleic acid sequence SEQ ID NO. 104871, preferably the coding region thereof, conferred the production of or the increase in valine compared with the wild type control.

The nucleic acid sequence of B3256 from Escherichia coli, e.g. as shown in the respective line in column 5 of Table I, application no. 6, has been published in B. And the activity of the gene product thereof is the activity of acetyl CoA carboxylase.

Accordingly, in one embodiment, the process of the present invention for producing leucine in a non-human organism, like a microorganism or a plant or a part thereof, comprises increasing or generating the activity of a gene product with the activity of a gene product conferring the activity of “acetyl CoA carboxylase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B3256, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3256, or a functional equivalent or a homolog thereof as depicted in column 8 of Table II, application no. 6, preferably a homolog or functional equivalent as depicted in column 8 of Table II B, application no. 6, and being depicted in the same respective line as said B3256, and preferably the activity is increased non-targeted, whereby the respective line discloses in column 7 the fine chemical leucine.

Accordingly, in one embodiment, the molecule which activity is to be increased in the process of the invention is the gene product with an activity as a “acetyl CoA carboxylase”, preferably it is the molecule of section (a) or (b) of this paragraph.

In particular, it was observed that in plants, especially in Arabidopsis thaliana, increasing or generating the activity of a gene product non-targeted with the activity of a “acetyl CoA carboxylase”, preferably being encoded by a gene comprising the nucleic acid sequence SEQ ID NO. 9492, preferably the coding region thereof, conferred the production of or the increase in leucine compared with the wild type control.

The nucleic acid sequence of B3352 from Escherichia coli, e.g. as shown in the respective line in column 5 of Table I, application no. 6, has been published in B. And the activity of the gene product thereof is the activity of ABC transporter ATP-binding protein.

Accordingly, in one embodiment, the process of the present invention for producing valine in a non-human organism, like a microorganism or a plant or a part thereof, comprises increasing or generating the activity of a gene product with the activity of a gene product conferring the activity of “ABC transporter ATP-binding protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B3352, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3352, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3352, or a functional equivalent or a homolog thereof as depicted in column 8 of Table II, application no. 6, preferably a homolog or functional equivalent as depicted in column 8 of Table II B, application no. 6, and being depicted in the same respective line as said B3352, and preferably the activity is increased non-targeted, whereby the respective line discloses in column 7 the fine chemical valine.

Accordingly, in one embodiment, the molecule which activity is to be increased in the process of the invention is the gene product with an activity as a “ABC transporter ATP-binding protein”, preferably it is the molecule of section (a) or (b) of this paragraph.

In particular, it was observed that in plants, especially in Arabidopsis thaliana, increasing or generating the activity of a gene product non-targeted with the activity of a “ABC transporter ATP-binding protein”, preferably being encoded by a gene comprising the nucleic acid sequence SEQ ID NO. 82572, preferably the coding region thereof, conferred the production of or the increase in valine compared with the wild type control.

The nucleic acid sequence of B3429 from Escherichia coli, e.g. as shown in the respective line in column 5 of Table I, application no. 6, has been published in B. And the activity of the gene product thereof is the activity of glycogen synthase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “glycogen synthase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B3429, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3429, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3429, or a functional equivalent or a homolog thereof as depicted in column 8 of Table II, application no. 6, preferably a homolog or functional equivalent as depicted in column 8 of Table II B, application no. 6, and being depicted in the same respective line as said B3429, and preferably the activity is increased mitochondrial, whereby the respective line discloses in column 7 the fine chemical isoleucine.

Accordingly, in one embodiment, the molecule which activity is to be increased in the process of the invention is the gene product with an activity as a “glycogen synthase”, preferably it is the molecule of section (a) or (b) of this paragraph.

In particular, it was observed that in plants, especially in Arabidopsis thaliana, increasing or generating the activity of a gene product mitochondrial with the activity of a “glycogen synthase”, preferably being encoded by a gene comprising the nucleic acid sequence SEQ ID NO. 94051, preferably the coding region thereof, conferred the production of or the increase in isoleucine compared with the wild type control.

The nucleic acid sequence of B3429 from Escherichia coli, e.g. as shown in the respective line in column 5 of Table I, application no. 6, has been published in B. And the activity of the gene product thereof is the activity of glycogen synthase.

Accordingly, in one embodiment, the process of the present invention for producing leucine in a non-human organism, like a microorganism or a plant or a part thereof, comprises increasing or generating the activity of a gene product with the activity of a gene product conferring the activity of “glycogen synthase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B3429, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3429, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3429, or a functional equivalent or a homolog thereof as depicted in column 8 of Table II, application no. 6, preferably a homolog or functional equivalent as depicted in column 8 of Table II B, application no. 6, and being depicted in the same respective line as said B3429, and preferably the activity is increased mitochondrial, whereby the respective line discloses in column 7 the fine chemical leucine.

Accordingly, in one embodiment, the molecule which activity is to be increased in the process of the invention is the gene product with an activity as a “glycogen synthase”, preferably it is the molecule of section (a) or (b) of this paragraph.

In particular, it was observed that in plants, especially in Arabidopsis thaliana, increasing or generating the activity of a gene product mitochondrial with the activity of a “glycogen synthase”, preferably being encoded by a gene comprising the nucleic acid sequence SEQ ID NO. 94051, preferably the coding region thereof, conferred the production of or the increase in leucine compared with the wild type control.

The nucleic acid sequence of B3490 from Escherichia coli, e.g. as shown in the respective line in column 5 of Table I, application no. 6, has been published in B. And the activity of the gene product thereof is the activity of b3490-protein.

Accordingly, in one embodiment, the process of the present invention for producing valine in a non-human organism, like a microorganism or a plant or a part thereof, comprises increasing or generating the activity of a gene product with the activity of a gene product conferring the activity of “b3490-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B3490, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3490, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3490, or a functional equivalent or a homolog thereof as depicted in column 8 of Table II, application no. 6, preferably a homolog or functional equivalent as depicted in column 8 of Table II B, application no. 6, and being depicted in the same respective line as said B3490, and preferably the activity is increased non-targeted, whereby the respective line discloses in column 7 the fine chemical valine.

Accordingly, in one embodiment, the molecule which activity is to be increased in the process of the invention is the gene product with an activity as a “b3490-protein”, preferably it is the molecule of section (a) or (b) of this paragraph.

In particular, it was observed that in plants, especially in Arabidopsis thaliana, increasing or generating the activity of a gene product non-targeted with the activity of a “b3490-protein”, preferably being encoded by a gene comprising the nucleic acid sequence SEQ ID NO. 105332, preferably the coding region thereof, conferred the production of or the increase in valine compared with the wild type control.

The nucleic acid sequence of B3572 from Escherichia coli, e.g. as shown in the respective line in column 5 of Table I, application no. 6, has been published in B. And the activity of the gene product thereof is the activity of valine-pyruvate transaminase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “valine-pyruvate transaminase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B3572, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3572, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3572, or a functional equivalent or a homolog thereof as depicted in column 8 of Table II, application no. 6, preferably a homolog or functional equivalent as depicted in column 8 of Table II B, application no. 6, and being depicted in the same respective line as said B3572, and preferably the activity is increased plastidic, whereby the respective line discloses in column 7 the fine chemical isoleucine.

Accordingly, in one embodiment, the molecule which activity is to be increased in the process of the invention is the gene product with an activity as a “valine-pyruvate transaminase”, preferably it is the molecule of section (a) or (b) of this paragraph.

In particular, it was observed that in plants, especially in Arabidopsis thaliana, increasing or generating the activity of a gene product plastidic with the activity of a “valine-pyruvate transaminase”, preferably being encoded by a gene comprising the nucleic acid sequence SEQ ID NO. 10172, preferably the coding region thereof, conferred the production of or the increase in isoleucine compared with the wild type control.

The nucleic acid sequence of B3572 from Escherichia coli, e.g. as shown in the respective line in column 5 of Table I, application no. 6, has been published in B. And the activity of the gene product thereof is the activity of valine-pyruvate transaminase.

Accordingly, in one embodiment, the process of the present invention for producing leucine in a non-human organism, like a microorganism or a plant or a part thereof, comprises increasing or generating the activity of a gene product with the activity of a gene product conferring the activity of “valine-pyruvate transaminase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B3572, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3572, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3572, or a functional equivalent or a homolog thereof as depicted in column 8 of Table II, application no. 6, preferably a homolog or functional equivalent as depicted in column 8 of Table II B, application no. 6, and being depicted in the same respective line as said B3572, and preferably the activity is increased plastidic, whereby the respective line discloses in column 7 the fine chemical leucine.

Accordingly, in one embodiment, the molecule which activity is to be increased in the process of the invention is the gene product with an activity as a “valine-pyruvate transaminase”, preferably it is the molecule of section (a) or (b) of this paragraph.

In particular, it was observed that in plants, especially in Arabidopsis thaliana, increasing or generating the activity of a gene product plastidic with the activity of a “valine-pyruvate transaminase”, preferably being encoded by a gene comprising the nucleic acid sequence SEQ ID NO. 10172, preferably the coding region thereof, conferred the production of or the increase in leucine compared with the wild type control.

The nucleic acid sequence of B3634 from Escherichia coli, e.g. as shown in the respective line in column 5 of Table I, application no. 6, has been published in B. And the activity of the gene product thereof is the activity of phosphopantetheine adenylyltransferase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “phosphopantetheine adenylyltransferase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B3634, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3634, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3634, or a functional equivalent or a homolog thereof as depicted in column 8 of Table II, application no. 6, preferably a homolog or functional equivalent as depicted in column 8 of Table II B, application no. 6, and being depicted in the same respective line as said B3634, and preferably the activity is increased non-targeted, whereby the respective line discloses in column 7 the fine chemical isoleucine.

Accordingly, in one embodiment, the molecule which activity is to be increased in the process of the invention is the gene product with an activity as a “phosphopantetheine adenylyltransferase”, preferably it is the molecule of section (a) or (b) of this paragraph.

In particular, it was observed that in plants, especially in Arabidopsis thaliana, increasing or generating the activity of a gene product non-targeted with the activity of a “phosphopantetheine adenylyltransferase”, preferably being encoded by a gene comprising the nucleic acid sequence SEQ ID NO. 42931, preferably the coding region thereof, conferred the production of or the increase in isoleucine compared with the wild type control.

The nucleic acid sequence of B3634 from Escherichia coli, e.g. as shown in the respective line in column 5 of Table I, application no. 6, has been published in B. And the activity of the gene product thereof is the activity of phosphopantetheine adenylyltransferase.

Accordingly, in one embodiment, the process of the present invention for producing leucine in a non-human organism, like a microorganism or a plant or a part thereof, comprises increasing or generating the activity of a gene product with the activity of a gene product conferring the activity of “phosphopantetheine adenylyltransferase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B3634, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3634, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3634, or a functional equivalent or a homolog thereof as depicted in column 8 of Table II, application no. 6, preferably a homolog or functional equivalent as depicted in column 8 of Table II B, application no. 6, and being depicted in the same respective line as said B3634, and preferably the activity is increased non-targeted, whereby the respective line discloses in column 7 the fine chemical leucine.

Accordingly, in one embodiment, the molecule which activity is to be increased in the process of the invention is the gene product with an activity as a “phosphopantetheine adenylyltransferase”, preferably it is the molecule of section (a) or (b) of this paragraph.

In particular, it was observed that in plants, especially in Arabidopsis thaliana, increasing or generating the activity of a gene product non-targeted with the activity of a “phosphopantetheine adenylyltransferase”, preferably being encoded by a gene comprising the nucleic acid sequence SEQ ID NO. 42931, preferably the coding region thereof, conferred the production of or the increase in leucine compared with the wild type control.

The nucleic acid sequence of B3634 from Escherichia coli, e.g. as shown in the respective line in column 5 of Table I, application no. 6, has been published in B. And the activity of the gene product thereof is the activity of phosphopantetheine adenylyltransferase.

Accordingly, in one embodiment, the process of the present invention for producing valine in a non-human organism, like a microorganism or a plant or a part thereof, comprises increasing or generating the activity of a gene product with the activity of a gene product conferring the activity of “phosphopantetheine adenylyltransferase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B3634, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3634, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3634, or a functional equivalent or a homolog thereof as depicted in column 8 of Table II, application no. 6, preferably a homolog or functional equivalent as depicted in column 8 of Table II B, application no. 6, and being depicted in the same respective line as said B3634, and preferably the activity is increased non-targeted, whereby the respective line discloses in column 7 the fine chemical valine.

Accordingly, in one embodiment, the molecule which activity is to be increased in the process of the invention is the gene product with an activity as a “phosphopantetheine adenylyltransferase”, preferably it is the molecule of section (a) or (b) of this paragraph.

In particular, it was observed that in plants, especially in Arabidopsis thaliana, increasing or generating the activity of a gene product non-targeted with the activity of a “phosphopantetheine adenylyltransferase”, preferably being encoded by a gene comprising the nucleic acid sequence SEQ ID NO. 42931, preferably the coding region thereof, conferred the production of or the increase in valine compared with the wild type control.

The nucleic acid sequence of B3670 from Escherichia coli, e.g. as shown in the respective line in column 5 of Table I, application no. 6, has been published in B. And the activity of the gene product thereof is the activity of acetolactate synthase.

Accordingly, in one embodiment, the process of the present invention for producing leucine in a non-human organism, like a microorganism or a plant or a part thereof, comprises increasing or generating the activity of a gene product with the activity of a gene product conferring the activity of “acetolactate synthase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B3670, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3670, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3670, or a functional equivalent or a homolog thereof as depicted in column 8 of Table II, application no. 6, preferably a homolog or functional equivalent as depicted in column 8 of Table II B, application no. 6, and being depicted in the same respective line as said B3670, and preferably the activity is increased plastidic, whereby the respective line discloses in column 7 the fine chemical leucine.

Accordingly, in one embodiment, the molecule which activity is to be increased in the process of the invention is the gene product with an activity as a “acetolactate synthase”, preferably it is the molecule of section (a) or (b) of this paragraph.

In particular, it was observed that in plants, especially in Arabidopsis thaliana, increasing or generating the activity of a gene product plastidic with the activity of a “acetolactate synthase”, preferably being encoded by a gene comprising the nucleic acid sequence SEQ ID NO. 83629, preferably the coding region thereof, conferred the production of or the increase in leucine compared with the wild type control.

The nucleic acid sequence of B3770 from Escherichia coli, e.g. as shown in the respective line in column 5 of Table I, application no. 6, has been published in B. And the activity of the gene product thereof is the activity of branched-chain amino-acid aminotransferase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “branched-chain amino-acid aminotransferase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B3770, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3770, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3770, or a functional equivalent or a homolog thereof as depicted in column 8 of Table II, application no. 6, preferably a homolog or functional equivalent as depicted in column 8 of Table II B, application no. 6, and being depicted in the same respective line as said B3770, and preferably the activity is increased plastidic, whereby the respective line discloses in column 7 the fine chemical isoleucine.

Accordingly, in one embodiment, the molecule which activity is to be increased in the process of the invention is the gene product with an activity as a “branched-chain amino-acid aminotransferase”, preferably it is the molecule of section (a) or (b) of this paragraph.

In particular, it was observed that in plants, especially in Arabidopsis thaliana, increasing or generating the activity of a gene product plastidic with the activity of a “branched-chain aminoacid aminotransferase”, preferably being encoded by a gene comprising the nucleic acid sequence SEQ ID NO. 105354, preferably the coding region thereof, conferred the production of or the increase in isoleucine compared with the wild type control.

The nucleic acid sequence of B3771 from Escherichia coli, e.g. as shown in the respective line in column 5 of Table I, application no. 6, has been published in B. And the activity of the gene product thereof is the activity of dihydroxyacid dehydratase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “dihydroxyacid dehydratase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B3771, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3771, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3771, or a functional equivalent or a homolog thereof as depicted in column 8 of Table II, application no. 6, preferably a homolog or functional equivalent as depicted in column 8 of Table II B, application no. 6, and being depicted in the same respective line as said B3771, and preferably the activity is increased non-targeted, whereby the respective line discloses in column 7 the fine chemical isoleucine.

Accordingly, in one embodiment, the molecule which activity is to be increased in the process of the invention is the gene product with an activity as a “dihydroxyacid dehydratase”, preferably it is the molecule of section (a) or (b) of this paragraph.

In particular, it was observed that in plants, especially in Arabidopsis thaliana, increasing or generating the activity of a gene product non-targeted with the activity of a “dihydroxyacid dehydratase”, preferably being encoded by a gene comprising the nucleic acid sequence SEQ ID NO. 10252, preferably the coding region thereof, conferred the production of or the increase in isoleucine compared with the wild type control.

The nucleic acid sequence of B3771 from Escherichia coli, e.g. as shown in the respective line in column 5 of Table I, application no. 6, has been published in B. And the activity of the gene product thereof is the activity of dihydroxyacid dehydratase.

Accordingly, in one embodiment, the process of the present invention for producing leucine in a non-human organism, like a microorganism or a plant or a part thereof, comprises increasing or generating the activity of a gene product with the activity of a gene product conferring the activity of “dihydroxyacid dehydratase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B3771, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3771, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3771, or a functional equivalent or a homolog thereof as depicted in column 8 of Table II, application no. 6, preferably a homolog or functional equivalent as depicted in column 8 of Table II B, application no. 6, and being depicted in the same respective line as said B3771, and preferably the activity is increased non-targeted, whereby the respective line discloses in column 7 the fine chemical leucine.

Accordingly, in one embodiment, the molecule which activity is to be increased in the process of the invention is the gene product with an activity as a “dihydroxyacid dehydratase”, preferably it is the molecule of section (a) or (b) of this paragraph.

In particular, it was observed that in plants, especially in Arabidopsis thaliana, increasing or generating the activity of a gene product non-targeted with the activity of a “dihydroxyacid dehydratase”, preferably being encoded by a gene comprising the nucleic acid sequence SEQ ID NO. 10252, preferably the coding region thereof, conferred the production of or the increase in leucine compared with the wild type control.

The nucleic acid sequence of B3771 from Escherichia coli, e.g. as shown in the respective line in column 5 of Table I, application no. 6, has been published in B. And the activity of the gene product thereof is the activity of dihydroxyacid dehydratase.

Accordingly, in one embodiment, the process of the present invention for producing leucine in a non-human organism, like a microorganism or a plant or a part thereof, comprises increasing or generating the activity of a gene product with the activity of a gene product conferring the activity of “dihydroxyacid dehydratase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B3771, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3771, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3771, or a functional equivalent or a homolog thereof as depicted in column 8 of Table II, application no. 6, preferably a homolog or functional equivalent as depicted in column 8 of Table II B, application no. 6, and being depicted in the same respective line as said B3771, and preferably the activity is increased plastidic, whereby the respective line discloses in column 7 the fine chemical leucine.

Accordingly, in one embodiment, the molecule which activity is to be increased in the process of the invention is the gene product with an activity as a “dihydroxyacid dehydratase”, preferably it is the molecule of section (a) or (b) of this paragraph.

In particular, it was observed that in plants, especially in Arabidopsis thaliana, increasing or generating the activity of a gene product plastidic with the activity of a “dihydroxyacid dehydratase”, preferably being encoded by a gene comprising the nucleic acid sequence SEQ ID NO. 10252, preferably the coding region thereof, conferred the production of or the increase in leucine compared with the wild type control.

The nucleic acid sequence of B3771 from Escherichia coli, e.g. as shown in the respective line in column 5 of Table I, application no. 6, has been published in B. And the activity of the gene product thereof is the activity of dihydroxyacid dehydratase.

Accordingly, in one embodiment, the process of the present invention for producing valine in a non-human organism, like a microorganism or a plant or a part thereof, comprises increasing or generating the activity of a gene product with the activity of a gene product conferring the activity of “dihydroxyacid dehydratase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B3771, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3771, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3771, or a functional equivalent or a homolog thereof as depicted in column 8 of Table II, application no. 6, preferably a homolog or functional equivalent as depicted in column 8 of Table II B, application no. 6, and being depicted in the same respective line as said B3771, and preferably the activity is increased non-targeted, whereby the respective line discloses in column 7 the fine chemical valine.

Accordingly, in one embodiment, the molecule which activity is to be increased in the process of the invention is the gene product with an activity as a “dihydroxyacid dehydratase”, preferably it is the molecule of section (a) or (b) of this paragraph.

In particular, it was observed that in plants, especially in Arabidopsis thaliana, increasing or generating the activity of a gene product non-targeted with the activity of a “dihydroxyacid dehydratase”, preferably being encoded by a gene comprising the nucleic acid sequence SEQ ID NO. 10252, preferably the coding region thereof, conferred the production of or the increase in valine compared with the wild type control.

The nucleic acid sequence of B3774 from Escherichia coli, e.g. as shown in the respective line in column 5 of Table I, application no. 6, has been published in B. And the activity of the gene product thereof is the activity of ketol-acid reductoisomerase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “ketol-acid reductoisomerase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B3774, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3774, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3774, or a functional equivalent or a homolog thereof as depicted in column 8 of Table II, application no. 6, preferably a homolog or functional equivalent as depicted in column 8 of Table II B, application no. 6, and being depicted in the same respective line as said B3774, and preferably the activity is increased non-targeted, whereby the respective line discloses in column 7 the fine chemical isoleucine.

Accordingly, in one embodiment, the molecule which activity is to be increased in the process of the invention is the gene product with an activity as a “ketol-acid reductoisomerase”, preferably it is the molecule of section (a) or (b) of this paragraph.

In particular, it was observed that in plants, especially in Arabidopsis thaliana, increasing or generating the activity of a gene product non-targeted with the activity of a “ketol-acid reductoisomerase”, preferably being encoded by a gene comprising the nucleic acid sequence SEQ ID NO. 94386, preferably the coding region thereof, conferred the production of or the increase in isoleucine compared with the wild type control.

The nucleic acid sequence of B3774 from Escherichia coli, e.g. as shown in the respective line in column 5 of Table I, application no. 6, has been published in B. And the activity of the gene product thereof is the activity of ketol-acid reductoisomerase.

Accordingly, in one embodiment, the process of the present invention for producing leucine in a non-human organism, like a microorganism or a plant or a part thereof, comprises increasing or generating the activity of a gene product with the activity of a gene product conferring the activity of “ketol-acid reductoisomerase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B3774, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3774, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3774, or a functional equivalent or a homolog thereof as depicted in column 8 of Table II, application no. 6, preferably a homolog or functional equivalent as depicted in column 8 of Table II B, application no. 6, and being depicted in the same respective line as said B3774, and preferably the activity is increased plastidic, whereby the respective line discloses in column 7 the fine chemical leucine.

Accordingly, in one embodiment, the molecule which activity is to be increased in the process of the invention is the gene product with an activity as a “ketol-acid reductoisomerase”, preferably it is the molecule of section (a) or (b) of this paragraph.

In particular, it was observed that in plants, especially in Arabidopsis thaliana, increasing or generating the activity of a gene product plastidic with the activity of a “ketol-acid reductoisomerase”, preferably being encoded by a gene comprising the nucleic acid sequence SEQ ID NO. 94386, preferably the coding region thereof, conferred the production of or the increase in leucine compared with the wild type control.

The nucleic acid sequence of B3813 from Escherichia coli, e.g. as shown in the respective line in column 5 of Table I, application no. 6, has been published in B. And the activity of the gene product thereof is the activity of DNA helicase II.

Accordingly, in one embodiment, the process of the present invention for producing isoleucine in a non-human organism, like a microorganism or a plant or a part thereof, comprises increasing or generating the activity of a gene product with the activity of a gene product conferring the activity of “DNA helicase II”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B3813, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3813, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3813, or a functional equivalent or a homolog thereof as depicted in column 8 of Table II, application no. 6, preferably a homolog or functional equivalent as depicted in column 8 of Table II B, application no. 6, and being depicted in the same respective line as said B3813, and preferably the activity is increased non-targeted, whereby the respective line discloses in column 7 the fine chemical isoleucine.

Accordingly, in one embodiment, the molecule which activity is to be increased in the process of the invention is the gene product with an activity as a “DNA helicase II”, preferably it is the molecule of section (a) or (b) of this paragraph.

In particular, it was observed that in plants, especially in Arabidopsis thaliana, increasing or generating the activity of a gene product non-targeted with the activity of a “DNA helicase II”, preferably being encoded by a gene comprising the nucleic acid sequence SEQ ID NO. 43839, preferably the coding region thereof, conferred the production of or the increase in isoleucine compared with the wild type control.

The nucleic acid sequence of B3813 from Escherichia coli, e.g. as shown in the respective line in column 5 of Table I, application no. 6, has been published in B. And the activity of the gene product thereof is the activity of DNA helicase II.

Accordingly, in one embodiment, the process of the present invention for producing leucine in a non-human organism, like a microorganism or a plant or a part thereof, comprises increasing or generating the activity of a gene product with the activity of a gene product conferring the activity of “DNA helicase II”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B3813, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3813, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3813, or a functional equivalent or a homolog thereof as depicted in column 8 of Table II, application no. 6, preferably a homolog or functional equivalent as depicted in column 8 of Table II B, application no. 6, and being depicted in the same respective line as said B3813, and preferably the activity is increased non-targeted, whereby the respective line discloses in column 7 the fine chemical leucine.

Accordingly, in one embodiment, the molecule which activity is to be increased in the process of the invention is the gene product with an activity as a “DNA helicase II”, preferably it is the molecule of section (a) or (b) of this paragraph.

In particular, it was observed that in plants, especially in Arabidopsis thaliana, increasing or generating the activity of a gene product non-targeted with the activity of a “DNA helicase II”, preferably being encoded by a gene comprising the nucleic acid sequence SEQ ID NO. 43839, preferably the coding region thereof, conferred the production of or the increase in leucine compared with the wild type control.

The nucleic acid sequence of B3813 from Escherichia coli, e.g. as shown in the respective line in column 5 of Table I, application no. 6, has been published in B. And the activity of the gene product thereof is the activity of DNA helicase II.

Accordingly, in one embodiment, the process of the present invention for producing valine in a non-human organism, like a microorganism or a plant or a part thereof, comprises increasing or generating the activity of a gene product with the activity of a gene product conferring the activity of “DNA helicase II”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B3813, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3813, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3813, or a functional equivalent or a homolog thereof as depicted in column 8 of Table II, application no. 6, preferably a homolog or functional equivalent as depicted in column 8 of Table II B, application no. 6, and being depicted in the same respective line as said B3813, and preferably the activity is increased non-targeted, whereby the respective line discloses in column 7 the fine chemical valine.

Accordingly, in one embodiment, the molecule which activity is to be increased in the process of the invention is the gene product with an activity as a “DNA helicase II”, preferably it is the molecule of section (a) or (b) of this paragraph.

In particular, it was observed that in plants, especially in Arabidopsis thaliana, increasing or generating the activity of a gene product non-targeted with the activity of a “DNA helicase II”, preferably being encoded by a gene comprising the nucleic acid sequence SEQ ID NO. 43839, preferably the coding region thereof, conferred the production of or the increase in valine compared with the wild type control.

The nucleic acid sequence of B3850 from Escherichia coli, e.g. as shown in the respective line in column 5 of Table I, application no. 6, has been published in B. And the activity of the gene product thereof is the activity of protoporphyrin oxidase.

Accordingly, in one embodiment, the process of the present invention for producing leucine in a non-human organism, like a microorganism or a plant or a part thereof, comprises increasing or generating the activity of a gene product with the activity of a gene product conferring the activity of “protoporphyrin oxidase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B3850, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3850, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3850, or a functional equivalent or a homolog thereof as depicted in column 8 of Table II, application no. 6, preferably a homolog or functional equivalent as depicted in column 8 of Table II B, application no. 6, and being depicted in the same respective line as said B3850, and preferably the activity is increased plastidic, whereby the respective line discloses in column 7 the fine chemical leucine.

Accordingly, in one embodiment, the molecule which activity is to be increased in the process of the invention is the gene product with an activity as a “protoporphyrin oxidase”, preferably it is the molecule of section (a) or (b) of this paragraph.

In particular, it was observed that in plants, especially in Arabidopsis thaliana, increasing or generating the activity of a gene product plastidic with the activity of a “protoporphyrin oxidase”, preferably being encoded by a gene comprising the nucleic acid sequence SEQ ID NO. 105602, preferably the coding region thereof, conferred the production of or the increase in leucine compared with the wild type control.

The nucleic acid sequence of B3908 from Escherichia coli, e.g. as shown in the respective line in column 5 of Table I, application no. 6, has been published in B. And the activity of the gene product thereof is the activity of superoxide dismutase.

Accordingly, in one embodiment, the process of the present invention for producing leucine in a non-human organism, like a microorganism or a plant or a part thereof, comprises increasing or generating the activity of a gene product with the activity of a gene product conferring the activity of “superoxide dismutase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B3908, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3908, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3908, or a functional equivalent or a homolog thereof as depicted in column 8 of Table II, application no. 6, preferably a homolog or functional equivalent as depicted in column 8 of Table II B, application no. 6, and being depicted in the same respective line as said B3908, and preferably the activity is increased plastidic, whereby the respective line discloses in column 7 the fine chemical leucine.

Accordingly, in one embodiment, the molecule which activity is to be increased in the process of the invention is the gene product with an activity as a “superoxide dismutase”, preferably it is the molecule of section (a) or (b) of this paragraph.

In particular, it was observed that in plants, especially in Arabidopsis thaliana, increasing or generating the activity of a gene product plastidic with the activity of a “superoxide dismutase”, preferably being encoded by a gene comprising the nucleic acid sequence SEQ ID NO. 105659, preferably the coding region thereof, conferred the production of or the increase in leucine compared with the wild type control.

The nucleic acid sequence of B3989 from Escherichia coli, e.g. as shown in the respective line in column 5 of Table I, application no. 6, has been published in B. And the activity of the gene product thereof is the activity of b3989-protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “b3989-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B3989, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3989, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3989, or a functional equivalent or a homolog thereof as depicted in column 8 of Table II, application no. 6, preferably a homolog or functional equivalent as depicted in column 8 of Table II B, application no. 6, and being depicted in the same respective line as said B3989, and preferably the activity is increased non-targeted, whereby the respective line discloses in column 7 the fine chemical isoleucine.

Accordingly, in one embodiment, the molecule which activity is to be increased in the process of the invention is the gene product with an activity as a “b3989-protein”, preferably it is the molecule of section (a) or (b) of this paragraph.

In particular, it was observed that in plants, especially in Arabidopsis thaliana, increasing or generating the activity of a gene product non-targeted with the activity of a “b3989-protein”, preferably being encoded by a gene comprising the nucleic acid sequence SEQ ID NO. 44372, preferably the coding region thereof, conferred the production of or the increase in isoleucine compared with the wild type control.

The nucleic acid sequence of B3989 from Escherichia coli, e.g. as shown in the respective line in column 5 of Table I, application no. 6, has been published in B. And the activity of the gene product thereof is the activity of b3989-protein.

Accordingly, in one embodiment, the process of the present invention for producing leucine in a non-human organism, like a microorganism or a plant or a part thereof, comprises increasing or generating the activity of a gene product with the activity of a gene product conferring the activity of “b3989-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B3989, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3989, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3989, or a functional equivalent or a homolog thereof as depicted in column 8 of Table II, application no. 6, preferably a homolog or functional equivalent as depicted in column 8 of Table II B, application no. 6, and being depicted in the same respective line as said B3989, and preferably the activity is increased non-targeted, whereby the respective line discloses in column 7 the fine chemical leucine.

Accordingly, in one embodiment, the molecule which activity is to be increased in the process of the invention is the gene product with an activity as a “b3989-protein”, preferably it is the molecule of section (a) or (b) of this paragraph.

In particular, it was observed that in plants, especially in Arabidopsis thaliana, increasing or generating the activity of a gene product non-targeted with the activity of a “b3989-protein”, preferably being encoded by a gene comprising the nucleic acid sequence SEQ ID NO. 44372, preferably the coding region thereof, conferred the production of or the increase in leucine compared with the wild type control.

The nucleic acid sequence of B3989 from Escherichia coli, e.g. as shown in the respective line in column 5 of Table I, application no. 6, has been published in B. And the activity of the gene product thereof is the activity of b3989-protein.

Accordingly, in one embodiment, the process of the present invention for producing valine in a non-human organism, like a microorganism or a plant or a part thereof, comprises increasing or generating the activity of a gene product with the activity of a gene product conferring the activity of “b3989-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B3989, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B3989, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B3989, or a functional equivalent or a homolog thereof as depicted in column 8 of Table II, application no. 6, preferably a homolog or functional equivalent as depicted in column 8 of Table II B, application no. 6, and being depicted in the same respective line as said B3989, and preferably the activity is increased non-targeted, whereby the respective line discloses in column 7 the fine chemical valine.

Accordingly, in one embodiment, the molecule which activity is to be increased in the process of the invention is the gene product with an activity as a “b3989-protein”, preferably it is the molecule of section (a) or (b) of this paragraph.

In particular, it was observed that in plants, especially in Arabidopsis thaliana, increasing or generating the activity of a gene product non-targeted with the activity of a “b3989-protein”, preferably being encoded by a gene comprising the nucleic acid sequence SEQ ID NO. 44372, preferably the coding region thereof, conferred the production of or the increase in valine compared with the wild type control.

The nucleic acid sequence of B4029 from Escherichia coli, e.g. as shown in the respective line in column 5 of Table I, application no. 6, has been published in B. And the activity of the gene product thereof is the activity of b4029-protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “b4029-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B4029, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B4029, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B4029, or a functional equivalent or a homolog thereof as depicted in column 8 of Table II, application no. 6, preferably a homolog or functional equivalent as depicted in column 8 of Table II B, application no. 6, and being depicted in the same respective line as said B4029, and preferably the activity is increased non-targeted, whereby the respective line discloses in column 7 the fine chemical isoleucine.

Accordingly, in one embodiment, the molecule which activity is to be increased in the process of the invention is the gene product with an activity as a “b4029-protein”, preferably it is the molecule of section (a) or (b) of this paragraph.

In particular, it was observed that in plants, especially in Arabidopsis thaliana, increasing or generating the activity of a gene product non-targeted with the activity of a “b4029-protein”, preferably being encoded by a gene comprising the nucleic acid sequence SEQ ID NO. 10740, preferably the coding region thereof, conferred the production of or the increase in isoleucine compared with the wild type control.

The nucleic acid sequence of B4029 from Escherichia coli, e.g. as shown in the respective line in column 5 of Table I, application no. 6, has been published in B. And the activity of the gene product thereof is the activity of b4029-protein.

Accordingly, in one embodiment, the process of the present invention for producing leucine in a non-human organism, like a microorganism or a plant or a part thereof, comprises increasing or generating the activity of a gene product with the activity of a gene product conferring the activity of “b4029-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B4029, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B4029, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B4029, or a functional equivalent or a homolog thereof as depicted in column 8 of Table II, application no. 6, preferably a homolog or functional equivalent as depicted in column 8 of Table II B, application no. 6, and being depicted in the same respective line as said B4029, and preferably the activity is increased non-targeted, whereby the respective line discloses in column 7 the fine chemical leucine.

Accordingly, in one embodiment, the molecule which activity is to be increased in the process of the invention is the gene product with an activity as a “b4029-protein”, preferably it is the molecule of section (a) or (b) of this paragraph.

In particular, it was observed that in plants, especially in Arabidopsis thaliana, increasing or generating the activity of a gene product non-targeted with the activity of a “b4029-protein”, preferably being encoded by a gene comprising the nucleic acid sequence SEQ ID NO. 10740, preferably the coding region thereof, conferred the production of or the increase in leucine compared with the wild type control.

The nucleic acid sequence of B4029 from Escherichia coli, e.g. as shown in the respective line in column 5 of Table I, application no. 6, has been published in B. And the activity of the gene product thereof is the activity of b4029-protein.

Accordingly, in one embodiment, the process of the present invention for producing valine in a non-human organism, like a microorganism or a plant or a part thereof, comprises increasing or generating the activity of a gene product with the activity of a gene product conferring the activity of “b4029-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B4029, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B4029, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B4029, or a functional equivalent or a homolog thereof as depicted in column 8 of Table II, application no. 6, preferably a homolog or functional equivalent as depicted in column 8 of Table II B, application no. 6, and being depicted in the same respective line as said B4029, and preferably the activity is increased non-targeted, whereby the respective line discloses in column 7 the fine chemical valine.

Accordingly, in one embodiment, the molecule which activity is to be increased in the process of the invention is the gene product with an activity as a “b4029-protein”, preferably it is the molecule of section (a) or (b) of this paragraph.

In particular, it was observed that in plants, especially in Arabidopsis thaliana, increasing or generating the activity of a gene product non-targeted with the activity of a “b4029-protein”, preferably being encoded by a gene comprising the nucleic acid sequence SEQ ID NO. 10740, preferably the coding region thereof, conferred the production of or the increase in valine compared with the wild type control.

The nucleic acid sequence of B4050 from Escherichia coli, e.g. as shown in the respective line in column 5 of Table I, application no. 6, has been published in B. And the activity of the gene product thereof is the activity of b4050-protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “b4050-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B4050, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B4050, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B4050, or a functional equivalent or a homolog thereof as depicted in column 8 of Table II, application no. 6, preferably a homolog or functional equivalent as depicted in column 8 of Table II B, application no. 6, and being depicted in the same respective line as said B4050, and preferably the activity is increased non-targeted, whereby the respective line discloses in column 7 the fine chemical isoleucine.

Accordingly, in one embodiment, the molecule which activity is to be increased in the process of the invention is the gene product with an activity as a “b4050-protein”, preferably it is the molecule of section (a) or (b) of this paragraph.

In particular, it was observed that in plants, especially in Arabidopsis thaliana, increasing or generating the activity of a gene product non-targeted with the activity of a “b4050-protein”, preferably being encoded by a gene comprising the nucleic acid sequence SEQ ID NO. 106532, preferably the coding region thereof, conferred the production of or the increase in isoleucine compared with the wild type control.

The nucleic acid sequence of B4056 from Escherichia coli, e.g. as shown in the respective line in column 5 of Table I, application no. 6, has been published in B. And the activity of the gene product thereof is the activity of b4056-protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “b4056-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B4056, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B4056, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B4056, or a functional equivalent or a homolog thereof as depicted in column 8 of Table II, application no. 6, preferably a homolog or functional equivalent as depicted in column 8 of Table II B, application no. 6, and being depicted in the same respective line as said B4056, and preferably the activity is increased non-targeted, whereby the respective line discloses in column 7 the fine chemical isoleucine.

Accordingly, in one embodiment, the molecule which activity is to be increased in the process of the invention is the gene product with an activity as a “b4056-protein”, preferably it is the molecule of section (a) or (b) of this paragraph.

In particular, it was observed that in plants, especially in Arabidopsis thaliana, increasing or generating the activity of a gene product non-targeted with the activity of a “b4056-protein”, preferably being encoded by a gene comprising the nucleic acid sequence SEQ ID NO. 94542, preferably the coding region thereof, conferred the production of or the increase in isoleucine compared with the wild type control.

The nucleic acid sequence of B4056 from Escherichia coli, e.g. as shown in the respective line in column 5 of Table I, application no. 6, has been published in B. And the activity of the gene product thereof is the activity of b4056-protein.

Accordingly, in one embodiment, the process of the present invention for producing leucine in a non-human organism, like a microorganism or a plant or a part thereof, comprises increasing or generating the activity of a gene product with the activity of a gene product conferring the activity of “b4056-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B4056, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B4056, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B4056, or a functional equivalent or a homolog thereof as depicted in column 8 of Table II, application no. 6, preferably a homolog or functional equivalent as depicted in column 8 of Table II B, application no. 6, and being depicted in the same respective line as said B4056, and preferably the activity is increased non-targeted, whereby the respective line discloses in column 7 the fine chemical leucine.

Accordingly, in one embodiment, the molecule which activity is to be increased in the process of the invention is the gene product with an activity as a “b4056-protein”, preferably it is the molecule of section (a) or (b) of this paragraph.

In particular, it was observed that in plants, especially in Arabidopsis thaliana, increasing or generating the activity of a gene product non-targeted with the activity of a “b4056-protein”, preferably being encoded by a gene comprising the nucleic acid sequence SEQ ID NO. 94542, preferably the coding region thereof, conferred the production of or the increase in leucine compared with the wild type control.

The nucleic acid sequence of B4256 from Escherichia coli, e.g. as shown in the respective line in column 5 of Table I, application no. 6, has been published in B. And the activity of the gene product thereof is the activity of acetyltransferase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B4256, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B4256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B4256, or a functional equivalent or a homolog thereof as depicted in column 8 of Table II, application no. 6, preferably a homolog or functional equivalent as depicted in column 8 of Table II B, application no. 6, and being depicted in the same respective line as said B4256, and preferably the activity is increased non-targeted, whereby the respective line discloses in column 7 the fine chemical isoleucine.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 45321, preferably the coding region thereof, conferred the production of or the increase in isoleucine compared with the wild type control.

The nucleic acid sequence of B4256 from Escherichia coli, e.g. as shown in the respective line in column 5 of Table I, application no. 6, has been published in B. And the activity of the gene product thereof is the activity of acetyltransferase.

Accordingly, in one embodiment, the process of the present invention for producing leucine in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B4256, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B4256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B4256, or a functional equivalent or a homolog thereof as depicted in column 8 of Table II, application no. 6, preferably a homolog or functional equivalent as depicted in column 8 of Table II B, application no. 6, and being depicted in the same respective line as said B4256, and preferably the activity is increased non-targeted, whereby the respective line discloses in column 7 the fine chemical leucine.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 45321, preferably the coding region thereof, conferred the production of or the increase in leucine compared with the wild type control.

The nucleic acid sequence of B4256 from Escherichia coli, e.g. as shown in the respective line in column 5 of Table I, application no. 6, has been published in B. And the activity of the gene product thereof is the activity of acetyltransferase.

Accordingly, in one embodiment, the process of the present invention for producing valine in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B4256, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B4256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B4256, or a functional equivalent or a homolog thereof as depicted in column 8 of Table II, application no. 6, preferably a homolog or functional equivalent as depicted in column 8 of Table II B, application no. 6, and being depicted in the same respective line as said B4256, and preferably the activity is increased non-targeted, whereby the respective line discloses in column 7 the fine chemical valine.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 45321, preferably the coding region thereof, conferred the production of or the increase in valine compared with the wild type control.

The nucleic acid sequence of B4263 from Escherichia coli, e.g. as shown in the respective line in column 5 of Table I, application no. 6, has been published in B. And the activity of the gene product thereof is the activity of b4263-protein.

Accordingly, in one embodiment, the process of the present invention for producing valine in a non-human organism, like a microorganism or a plant or a part thereof, comprises increasing or generating the activity of a gene product with the activity of a gene product conferring the activity of “b4263-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B4263, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B4263, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B4263, or a functional equivalent or a homolog thereof as depicted in column 8 of Table II, application no. 6, preferably a homolog or functional equivalent as depicted in column 8 of Table II B, application no. 6, and being depicted in the same respective line as said B4263, and preferably the activity is increased plastidic, whereby the respective line discloses in column 7 the fine chemical valine.

Accordingly, in one embodiment, the molecule which activity is to be increased in the process of the invention is the gene product with an activity as a “b4263-protein”, preferably it is the molecule of section (a) or (b) of this paragraph.

In particular, it was observed that in plants, especially in Arabidopsis thaliana, increasing or generating the activity of a gene product plastidic with the activity of a “b4263-protein”, preferably being encoded by a gene comprising the nucleic acid sequence SEQ ID NO. 106538, preferably the coding region thereof, conferred the production of or the increase in valine compared with the wild type control.

The nucleic acid sequence of B4321 from Escherichia coli, e.g. as shown in the respective line in column 5 of Table I, application no. 6, has been published in B. And the activity of the gene product thereof is the activity of gluconate transport system permease 3.

Accordingly, in one embodiment, the process of the present invention for producing isoleucine in a non-human organism, like a microorganism or a plant or a part thereof, comprises increasing or generating the activity of a gene product with the activity of a gene product conferring the activity of “gluconate transport system permease 3”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B4321, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B4321, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B4321, or a functional equivalent or a homolog thereof as depicted in column 8 of Table II, application no. 6, preferably a homolog or functional equivalent as depicted in column 8 of Table II B, application no. 6, and being depicted in the same respective line as said B4321, and preferably the activity is increased non-targeted, whereby the respective line discloses in column 7 the fine chemical isoleucine.

Accordingly, in one embodiment, the molecule which activity is to be increased in the process of the invention is the gene product with an activity as a “gluconate transport system permease 3”, preferably it is the molecule of section (a) or (b) of this paragraph.

In particular, it was observed that in plants, especially in Arabidopsis thaliana, increasing or generating the activity of a gene product non-targeted with the activity of a “gluconate transport system permease 3”, preferably being encoded by a gene comprising the nucleic acid sequence SEQ ID NO. 45394, preferably the coding region thereof, conferred the production of or the increase in isoleucine compared with the wild type control.

The nucleic acid sequence of B4321 from Escherichia coli, e.g. as shown in the respective line in column 5 of Table I, application no. 6, has been published in B. And the activity of the gene product thereof is the activity of gluconate transport system permease 3.

Accordingly, in one embodiment, the process of the present invention for producing leucine in a non-human organism, like a microorganism or a plant or a part thereof, comprises increasing or generating the activity of a gene product with the activity of a gene product conferring the activity of “gluconate transport system permease 3”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said B4321, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B4321, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B4321, or a functional equivalent or a homolog thereof as depicted in column 8 of Table II, application no. 6, preferably a homolog or functional equivalent as depicted in column 8 of Table II B, application no. 6, and being depicted in the same respective line as said B4321, and preferably the activity is increased non-targeted, whereby the respective line discloses in column 7 the fine chemical leucine.

Accordingly, in one embodiment, the molecule which activity is to be increased in the process of the invention is the gene product with an activity as a “gluconate transport system permease 3”, preferably it is the molecule of section (a) or (b) of this paragraph.

In particular, it was observed that in plants, especially in Arabidopsis thaliana, increasing or generating the activity of a gene product non-targeted with the activity of a “gluconate transport system permease 3”, preferably being encoded by a gene comprising the nucleic acid sequence SEQ ID NO. 45394, preferably the coding region thereof, conferred the production of or the increase in leucine compared with the wild type control.

The nucleic acid sequence of B4321 from Escherichia coli, e.g. as shown in the respective line in column 5 of Table I, application no. 6, has been published in B. And the activity of the gene product thereof is the activity of gluconate transport system permease 3.

Accordingly, in one embodiment, the process of the present invention for producing valine in a non-human organism, like a microorganism or a plant or a part thereof, comprises increasing or generating the activity of a gene product with the activity of a gene product conferring the activity of “gluconate transport system permease 3”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said B4321, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B4321, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B4321, or a functional equivalent or a homolog thereof as depicted in column 8 of Table II, application no. 6, preferably a homolog or functional equivalent as depicted in column 8 of Table II B, application no. 6, and being depicted in the same respective line as said B4321, and preferably the activity is increased non-targeted, whereby the respective line discloses in column 7 the fine chemical valine.

Accordingly, in one embodiment, the molecule which activity is to be increased in the process of the invention is the gene product with an activity as a “gluconate transport system permease 3”, preferably it is the molecule of section (a) or (b) of this paragraph.

In particular, it was observed that in plants, especially in Arabidopsis thaliana, increasing or generating the activity of a gene product non-targeted with the activity of a “gluconate transport system permease 3”, preferably being encoded by a gene comprising the nucleic acid sequence SEQ ID NO. 45394, preferably the coding region thereof, conferred the production of or the increase in valine compared with the wild type control.

The nucleic acid sequence of B4384 from Escherichia coli, e.g. as shown in the respective line in column 5 of Table I, application no. 6, has been published in B. And the activity of the gene product thereof is the activity of purine-nucleoside phosphorylase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “purine-nucleoside phosphorylase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said B4384, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said B4384, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said B4384, or a functional equivalent or a homolog thereof as depicted in column 8 of Table II, application no. 6, preferably a homolog or functional equivalent as depicted in column 8 of Table II B, application no. 6, and being depicted in the same respective line as said B4384, and preferably the activity is increased non-targeted, whereby the respective line discloses in column 7 the fine chemical isoleucine.

Accordingly, in one embodiment, the molecule which activity is to be increased in the process of the invention is the gene product with an activity as a “purine-nucleoside phosphorylase”, preferably it is the molecule of section (a) or (b) of this paragraph.

In particular, it was observed that in plants, especially in Arabidopsis thaliana, increasing or generating the activity of a gene product non-targeted with the activity of a “purine-nucleoside phosphorylase”, preferably being encoded by a gene comprising the nucleic acid sequence SEQ ID NO. 45556, preferably the coding region thereof, conferred the production of or the increase in isoleucine compared with the wild type control.

The nucleic acid sequence of C_pp004096192r from Physcomitrella patens, e.g. as shown in the respective line in column 5 of Table I, application no. 6, is unpublished. And the activity of the gene product thereof is the activity of thioredoxin.

Accordingly, in one embodiment, the process of the present invention for producing isoleucine in a non-human organism, like a microorganism or a plant or a part thereof, comprises increasing or generating the activity of a gene product with the activity of a gene product conferring the activity of “thioredoxin”, especially from Physcomitrella patens or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said C_pp004096192r, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said C_pp004096192r, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said

C_pp004096192r, or a functional equivalent or a homolog thereof as depicted in column 8 of Table II, application no. 6, preferably a homolog or functional equivalent as depicted in column 8 of Table II B, application no. 6, and being depicted in the same respective line as said C_pp004096192r, and preferably the activity is increased non-targeted, whereby the respective line discloses in column 7 the fine chemical isoleucine.

Accordingly, in one embodiment, the molecule which activity is to be increased in the process of the invention is the gene product with an activity as a “thioredoxin”, preferably it is the molecule of section (a) or (b) of this paragraph.

In particular, it was observed that in plants, especially in Arabidopsis thaliana, increasing or generating the activity of a gene product non-targeted with the activity of a “thioredoxin”, preferably being encoded by a gene comprising the nucleic acid sequence SEQ ID NO. 45757, preferably the coding region thereof, conferred the production of or the increase in isoleucine compared with the wild type control.

The nucleic acid sequence of C_pp004096192r from Physcomitrella patens, e.g. as shown in the respective line in column 5 of Table I, application no. 6, is unpublished. And the activity of the gene product thereof is the activity of thioredoxin.

Accordingly, in one embodiment, the process of the present invention for producing leucine in a non-human organism, like a microorganism or a plant or a part thereof, comprises increasing or generating the activity of a gene product with the activity of a gene product conferring the activity of “thioredoxin”, especially from Physcomitrella patens or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said C_pp004096192r, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said C_pp004096192r, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said C_pp004096192r, or a functional equivalent or a homolog thereof as depicted in column 8 of Table II, application no. 6, preferably a homolog or functional equivalent as depicted in column 8 of Table II B, application no. 6, and being depicted in the same respective line as said C_pp004096192r, and preferably the activity is increased non-targeted, whereby the respective line discloses in column 7 the fine chemical leucine.

Accordingly, in one embodiment, the molecule which activity is to be increased in the process of the invention is the gene product with an activity as a “thioredoxin”, preferably it is the molecule of section (a) or (b) of this paragraph.

In particular, it was observed that in plants, especially in Arabidopsis thaliana, increasing or generating the activity of a gene product non-targeted with the activity of a “thioredoxin”, preferably being encoded by a gene comprising the nucleic acid sequence SEQ ID NO. 45757, preferably the coding region thereof, conferred the production of or the increase in leucine compared with the wild type control.

The nucleic acid sequence of C_pp004096192r from Physcomitrella patens, e.g. as shown in the respective line in column 5 of Table I, application no. 6, is unpublished. And the activity of the gene product thereof is the activity of thioredoxin.

Accordingly, in one embodiment, the process of the present invention for producing valine in a non-human organism, like a microorganism or a plant or a part thereof, comprises increasing or generating the activity of a gene product with the activity of a gene product conferring the activity of “thioredoxin”, especially from Physcomitrella patens or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said C_pp004096192r, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said C_pp004096192r, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said C_pp004096192r, or a functional equivalent or a homolog thereof as depicted in column 8 of Table II, application no. 6, preferably a homolog or functional equivalent as depicted in column 8 of Table II B, application no. 6, and being depicted in the same respective line as said C_pp004096192r, and preferably the activity is increased non-targeted, whereby the respective line discloses in column 7 the fine chemical valine.

Accordingly, in one embodiment, the molecule which activity is to be increased in the process of the invention is the gene product with an activity as a “thioredoxin”, preferably it is the molecule of section (a) or (b) of this paragraph.

In particular, it was observed that in plants, especially in Arabidopsis thaliana, increasing or generating the activity of a gene product non-targeted with the activity of a “thioredoxin”, preferably being encoded by a gene comprising the nucleic acid sequence SEQ ID NO. 45757, preferably the coding region thereof, conferred the production of or the increase in valine compared with the wild type control.

The nucleic acid sequence of GM02LC11114 from Glycine max, e.g. as shown in the respective line in column 5 of Table I, application no. 6, is unpublished. And the activity of the gene product thereof is the activity of GM02LC11114-protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “GM02LC11114-protein”, especially from Glycine max or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said GM02LC11114, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said GM02LC11114, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said GM02LC11114, or a functional equivalent or a homolog thereof as depicted in column 8 of Table II, application no. 6, preferably a homolog or functional equivalent as depicted in column 8 of Table II B, application no. 6, and being depicted in the same respective line as said GM02LC11114, and preferably the activity is increased non-targeted, whereby the respective line discloses in column 7 the fine chemical isoleucine.

Accordingly, in one embodiment, the molecule which activity is to be increased in the process of the invention is the gene product with an activity as a “GM02LC11114-protein”, preferably it is the molecule of section (a) or (b) of this paragraph.

In particular, it was observed that in plants, especially in Arabidopsis thaliana, increasing or generating the activity of a gene product non-targeted with the activity of a “GM02LC11114-protein”, preferably being encoded by a gene comprising the nucleic acid sequence SEQ ID NO. 45795, preferably the coding region thereof, conferred the production of or the increase in isoleucine compared with the wild type control.

The nucleic acid sequence of GM02LC11114 from Glycine max, e.g. as shown in the respective line in column 5 of Table I, application no. 6, is unpublished. And the activity of the gene product thereof is the activity of GM02LC11114-protein.

Accordingly, in one embodiment, the process of the present invention for producing leucine in a non-human organism, like a microorganism or a plant or a part thereof, comprises increasing or generating the activity of a gene product with the activity of a gene product conferring the activity of “GM02LC11114-protein”, especially from Glycine max or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said GM02LC11114, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said GM02LC11114, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said GM02LC11114, or a functional equivalent or a homolog thereof as depicted in column 8 of Table II, application no. 6, preferably a homolog or functional equivalent as depicted in column 8 of Table II B, application no. 6, and being depicted in the same respective line as said GM02LC11114, and preferably the activity is increased non-targeted, whereby the respective line discloses in column 7 the fine chemical leucine.

Accordingly, in one embodiment, the molecule which activity is to be increased in the process of the invention is the gene product with an activity as a “GM02LC11114-protein”, preferably it is the molecule of section (a) or (b) of this paragraph.

In particular, it was observed that in plants, especially in Arabidopsis thaliana, increasing or generating the activity of a gene product non-targeted with the activity of a “GM02LC11114-protein”, preferably being encoded by a gene comprising the nucleic acid sequence SEQ ID NO. 45795, preferably the coding region thereof, conferred the production of or the increase in leucine compared with the wild type control.

The nucleic acid sequence of GM02LC12622 from Glycine max, e.g. as shown in the respective line in column 5 of Table I, application no. 6, is unpublished. And the activity of the gene product thereof is the activity of glutaredoxin.

Accordingly, in one embodiment, the process of the present invention for producing isoleucine in a non-human organism, like a microorganism or a plant or a part thereof, comprises increasing or generating the activity of a gene product with the activity of a gene product conferring the activity of “glutaredoxin”, especially from Glycine max or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said GM02LC12622, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said GM02LC12622, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said GM02LC12622, or a functional equivalent or a homolog thereof as depicted in column 8 of Table II, application no. 6, preferably a homolog or functional equivalent as depicted in column 8 of Table II B, application no. 6, and being depicted in the same respective line as said GM02LC12622, and preferably the activity is increased non-targeted, whereby the respective line discloses in column 7 the fine chemical isoleucine.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 10811, preferably the coding region thereof, conferred the production of or the increase in isoleucine compared with the wild type control.

The nucleic acid sequence of GM02LC12622 from Glycine max, e.g. as shown in the respective line in column 5 of Table I, application no. 6, is unpublished. And the activity of the gene product thereof is the activity of glutaredoxin.

Accordingly, in one embodiment, the process of the present invention for producing leucine in a non-human organism, like a microorganism or a plant or a part thereof, comprises increasing or generating the activity of a gene product with the activity of a gene product conferring the activity of “glutaredoxin”, especially from Glycine max or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said GM02LC12622, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said GM02LC12622, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said GM02LC12622, or a functional equivalent or a homolog thereof as depicted in column 8 of Table II, application no. 6, preferably a homolog or functional equivalent as depicted in column 8 of Table II B, application no. 6, and being depicted in the same respective line as said GM02LC12622, and preferably the activity is increased non-targeted, whereby the respective line discloses in column 7 the fine chemical leucine.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 10811, preferably the coding region thereof, conferred the production of or the increase in leucine compared with the wild type control.

The nucleic acid sequence of GM02LC12622 from Glycine max, e.g. as shown in the respective line in column 5 of Table I, application no. 6, is unpublished. And the activity of the gene product thereof is the activity of glutaredoxin.

Accordingly, in one embodiment, the process of the present invention for producing valine in a non-human organism, like a microorganism or a plant or a part thereof, comprises increasing or generating the activity of a gene product with the activity of a gene product conferring the activity of “glutaredoxin”, especially from Glycine max or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said GM02LC12622, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said GM02LC12622, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said GM02LC12622, or a functional equivalent or a homolog thereof as depicted in column 8 of Table II, application no. 6, preferably a homolog or functional equivalent as depicted in column 8 of Table II B, application no. 6, and being depicted in the same respective line as said GM02LC12622, and preferably the activity is increased non-targeted, whereby the respective line discloses in column 7 the fine chemical valine.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 10811, preferably the coding region thereof, conferred the production of or the increase in valine compared with the wild type control.

The nucleic acid sequence of GM02LC15313 from Glycine max, e.g. as shown in the respective line in column 5 of Table I, application no. 6, is unpublished. And the activity of the gene product thereof is the activity of histone H2A.

Accordingly, in one embodiment, the process of the present invention for producing isoleucine in a non-human organism, like a microorganism or a plant or a part thereof, comprises increasing or generating the activity of a gene product with the activity of a gene product conferring the activity of “histone H2A”, especially from Glycine max or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said GM02LC15313, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said GM02LC15313, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said GM02LC15313, or a functional equivalent or a homolog thereof as depicted in column 8 of Table II, application no. 6, preferably a homolog or functional equivalent as depicted in column 8 of Table II B, application no. 6, and being depicted in the same respective line as said GM02LC15313, and preferably the activity is increased non-targeted, whereby the respective line discloses in column 7 the fine chemical isoleucine.

Accordingly, in one embodiment, the molecule which activity is to be increased in the process of the invention is the gene product with an activity as a “histone H2A”, preferably it is the molecule of section (a) or (b) of this paragraph.

In particular, it was observed that in plants, especially in Arabidopsis thaliana, increasing or generating the activity of a gene product non-targeted with the activity of a “histone H2A”, preferably being encoded by a gene comprising the nucleic acid sequence SEQ ID NO. 45897, preferably the coding region thereof, conferred the production of or the increase in isoleucine compared with the wild type control.

The nucleic acid sequence of GM02LC15313 from Glycine max, e.g. as shown in the respective line in column 5 of Table I, application no. 6, is unpublished. And the activity of the gene product thereof is the activity of histone H2A.

Accordingly, in one embodiment, the process of the present invention for producing leucine in a non-human organism, like a microorganism or a plant or a part thereof, comprises increasing or generating the activity of a gene product with the activity of a gene product conferring the activity of “histone H2A”, especially from Glycine max or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said GM02LC15313, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said GM02LC15313, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said GM02LC15313, or a functional equivalent or a homolog thereof as depicted in column 8 of Table II, application no. 6, preferably a homolog or functional equivalent as depicted in column 8 of Table II B, application no. 6, and being depicted in the same respective line as said GM02LC15313, and preferably the activity is increased non-targeted, whereby the respective line discloses in column 7 the fine chemical leucine.

Accordingly, in one embodiment, the molecule which activity is to be increased in the process of the invention is the gene product with an activity as a “histone H2A”, preferably it is the molecule of section (a) or (b) of this paragraph.

In particular, it was observed that in plants, especially in Arabidopsis thaliana, increasing or generating the activity of a gene product non-targeted with the activity of a “histone H2A”, preferably being encoded by a gene comprising the nucleic acid sequence SEQ ID NO. 45897, preferably the coding region thereof, conferred the production of or the increase in leucine compared with the wild type control.

The nucleic acid sequence of GM02LC15313 from Glycine max, e.g. as shown in the respective line in column 5 of Table I, application no. 6, is unpublished. And the activity of the gene product thereof is the activity of histone H2A.

Accordingly, in one embodiment, the process of the present invention for producing valine in a non-human organism, like a microorganism or a plant or a part thereof, comprises increasing or generating the activity of a gene product with the activity of a gene product conferring the activity of “histone H2A”, especially from Glycine max or its functional equivalent or its homolog, e.g.

the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said GM02LC15313, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said GM02LC15313, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said GM02LC15313, or a functional equivalent or a homolog thereof as depicted in column 8 of Table II, application no. 6, preferably a homolog or functional equivalent as depicted in column 8 of Table II B, application no. 6, and being depicted in the same respective line as said GM02LC15313, and preferably the activity is increased non-targeted, whereby the respective line discloses in column 7 the fine chemical valine.

Accordingly, in one embodiment, the molecule which activity is to be increased in the process of the invention is the gene product with an activity as a “histone H2A”, preferably it is the molecule of section (a) or (b) of this paragraph.

In particular, it was observed that in plants, especially in Arabidopsis thaliana, increasing or generating the activity of a gene product non-targeted with the activity of a “histone H2A”, preferably being encoded by a gene comprising the nucleic acid sequence SEQ ID NO. 45897, preferably the coding region thereof, conferred the production of or the increase in valine compared with the wild type control.

The nucleic acid sequence of GM02LC17384 from Glycine max, e.g. as shown in the respective line in column 5 of Table I, application no. 6, is unpublished. And the activity of the gene product thereof is the activity of vacuolar ATPase subunit.

Accordingly, in one embodiment, the process of the present invention for producing valine in a non-human organism, like a microorganism or a plant or a part thereof, comprises increasing or generating the activity of a gene product with the activity of a gene product conferring the activity of “vacuolar ATPase subunit”, especially from Glycine max or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said GM02LC17384, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said GM02LC17384, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said GM02LC17384, or a functional equivalent or a homolog thereof as depicted in column 8 of Table II, application no. 6, preferably a homolog or functional equivalent as depicted in column 8 of Table II B, application no. 6, and being depicted in the same respective line as said GM02LC17384, and preferably the activity is increased non-targeted, whereby the respective line discloses in column 7 the fine chemical valine.

Accordingly, in one embodiment, the molecule which activity is to be increased in the process of the invention is the gene product with an activity as a “vacuolar ATPase subunit”, preferably it is the molecule of section (a) or (b) of this paragraph.

In particular, it was observed that in plants, especially in Arabidopsis thaliana, increasing or generating the activity of a gene product non-targeted with the activity of a “vacuolar ATPase subunit”, preferably being encoded by a gene comprising the nucleic acid sequence SEQ ID NO. 106688, preferably the coding region thereof, conferred the production of or the increase in valine compared with the wild type control.

The nucleic acid sequence of GM02LC17556 from Glycine max, e.g. as shown in the respective line in column 5 of Table I, application no. 6, is unpublished. And the activity of the gene product thereof is the activity of RNA binding protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “RNA binding protein”, especially from Glycine max or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said GM02LC17556, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said GM02LC17556, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said GM02LC17556, or a functional equivalent or a homolog thereof as depicted in column 8 of Table II, application no. 6, preferably a homolog or functional equivalent as depicted in column 8 of Table II B, application no. 6, and being depicted in the same respective line as said GM02LC17556, and preferably the activity is increased non-targeted, whereby the respective line discloses in column 7 the fine chemical isoleucine.

Accordingly, in one embodiment, the molecule which activity is to be increased in the process of the invention is the gene product with an activity as a “RNA binding protein”, preferably it is the molecule of section (a) or (b) of this paragraph.

In particular, it was observed that in plants, especially in Arabidopsis thaliana, increasing or generating the activity of a gene product non-targeted with the activity of a “RNA binding protein”, preferably being encoded by a gene comprising the nucleic acid sequence SEQ ID NO. 46515, preferably the coding region thereof, conferred the production of or the increase in isoleucine compared with the wild type control.

The nucleic acid sequence of GM02LC17556 from Glycine max, e.g. as shown in the respective line in column 5 of Table I, application no. 6, is unpublished. And the activity of the gene product thereof is the activity of RNA binding protein.

Accordingly, in one embodiment, the process of the present invention for producing leucine in a non-human organism, like a microorganism or a plant or a part thereof, comprises increasing or generating the activity of a gene product with the activity of a gene product conferring the activity of “RNA binding protein”, especially from Glycine max or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said GM02LC17556, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said GM02LC17556, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said GM02LC17556, or a functional equivalent or a homolog thereof as depicted in column 8 of Table II, application no. 6, preferably a homolog or functional equivalent as depicted in column 8 of Table II B, application no. 6, and being depicted in the same respective line as said GM02LC17556, and preferably the activity is increased non-targeted, whereby the respective line discloses in column 7 the fine chemical leucine.

Accordingly, in one embodiment, the molecule which activity is to be increased in the process of the invention is the gene product with an activity as a “RNA binding protein”, preferably it is the molecule of section (a) or (b) of this paragraph.

In particular, it was observed that in plants, especially in Arabidopsis thaliana, increasing or generating the activity of a gene product non-targeted with the activity of a “RNA binding protein”, preferably being encoded by a gene comprising the nucleic acid sequence SEQ ID NO. 46515, preferably the coding region thereof, conferred the production of or the increase in leucine compared with the wild type control.

The nucleic acid sequence of GM02LC17556 from Glycine max, e.g. as shown in the respective line in column 5 of Table I, application no. 6, is unpublished. And the activity of the gene product thereof is the activity of RNA binding protein.

Accordingly, in one embodiment, the process of the present invention for producing valine in a non-human organism, like a microorganism or a plant or a part thereof, comprises increasing or generating the activity of a gene product with the activity of a gene product conferring the activity of “RNA binding protein”, especially from Glycine max or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said GM02LC17556, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said GM02LC17556, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said GM02LC17556, or a functional equivalent or a homolog thereof as depicted in column 8 of Table II, application no. 6, preferably a homolog or functional equivalent as depicted in column 8 of Table II B, application no. 6, and being depicted in the same respective line as said GM02LC17556, and preferably the activity is increased non-targeted, whereby the respective line discloses in column 7 the fine chemical valine.

Accordingly, in one embodiment, the molecule which activity is to be increased in the process of the invention is the gene product with an activity as a “RNA binding protein”, preferably it is the molecule of section (a) or (b) of this paragraph.

In particular, it was observed that in plants, especially in Arabidopsis thaliana, increasing or generating the activity of a gene product non-targeted with the activity of a “RNA binding protein”, preferably being encoded by a gene comprising the nucleic acid sequence SEQ ID NO. 46515, preferably the coding region thereof, conferred the production of or the increase in valine compared with the wild type control.

The nucleic acid sequence of GM02LC21368 from Glycine max, e.g. as shown in the respective line in column 5 of Table I, application no. 6, is unpublished. And the activity of the gene product thereof is the activity of transcription factor.

Accordingly, in one embodiment, the process of the present invention for producing isoleucine in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said GM02LC21368, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said GM02LC21368, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said GM02LC21368, or a functional equivalent or a homolog thereof as depicted in column 8 of Table II, application no. 6, preferably a homolog or functional equivalent as depicted in column 8 of Table II B, application no. 6, and being depicted in the same respective line as said GM02LC21368, and preferably the activity is increased non-targeted, whereby the respective line discloses in column 7 the fine chemical isoleucine.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 84198, preferably the coding region thereof, conferred the production of or the increase in isoleucine compared with the wild type control.

The nucleic acid sequence of GM02LC21368 from Glycine max, e.g. as shown in the respective line in column 5 of Table I, application no. 6, is unpublished. And the activity of the gene product thereof is the activity of transcription factor.

Accordingly, in one embodiment, the process of the present invention for producing leucine in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said GM02LC21368, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said GM02LC21368, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said GM02LC21368, or a functional equivalent or a homolog thereof as depicted in column 8 of Table II, application no. 6, preferably a homolog or functional equivalent as depicted in column 8 of Table II B, application no. 6, and being depicted in the same respective line as said GM02LC21368, and preferably the activity is increased non-targeted, whereby the respective line discloses in column 7 the fine chemical leucine.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 84198, preferably the coding region thereof, conferred the production of or the increase in leucine compared with the wild type control.

The nucleic acid sequence of GM02LC21368 from Glycine max, e.g. as shown in the respective line in column 5 of Table I, application no. 6, is unpublished. And the activity of the gene product thereof is the activity of transcription factor.

Accordingly, in one embodiment, the process of the present invention for producing valine in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said GM02LC21368, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said GM02LC21368, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said GM02LC21368, or a functional equivalent or a homolog thereof as depicted in column 8 of Table II, application no. 6, preferably a homolog or functional equivalent as depicted in column 8 of Table II B, application no. 6, and being depicted in the same respective line as said GM02LC21368, and preferably the activity is increased non-targeted, whereby the respective line discloses in column 7 the fine chemical valine.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 84198, preferably the coding region thereof, conferred the production of or the increase in valine compared with the wild type control.

The nucleic acid sequence of GM02LC44512 from Glycine max, e.g. as shown in the respective line in column 5 of Table I, application no. 6, is unpublished. And the activity of the gene product thereof is the activity of cyclin D.

Accordingly, in one embodiment, the process of the present invention for producing isoleucine in a non-human organism, like a microorganism or a plant or a part thereof, comprises increasing or generating the activity of a gene product with the activity of a gene product conferring the activity of “cyclin D”, especially from Glycine max or its functional equivalent or its homolog, e.g.

the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said GM02LC44512, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said GM02LC44512, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said GM02LC44512, or a functional equivalent or a homolog thereof as depicted in column 8 of Table II, application no. 6, preferably a homolog or functional equivalent as depicted in column 8 of Table II B, application no. 6, and being depicted in the same respective line as said GM02LC44512, and preferably the activity is increased non-targeted, whereby the respective line discloses in column 7 the fine chemical isoleucine.

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 46850, preferably the coding region thereof, conferred the production of or the increase in isoleucine compared with the wild type control.

The nucleic acid sequence of GM02LC44512 from Glycine max, e.g. as shown in the respective line in column 5 of Table I, application no. 6, is unpublished. And the activity of the gene product thereof is the activity of cyclin D.

Accordingly, in one embodiment, the process of the present invention for producing leucine in a non-human organism, like a microorganism or a plant or a part thereof, comprises increasing or generating the activity of a gene product with the activity of a gene product conferring the activity of “cyclin D”, especially from Glycine max or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said GM02LC44512, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said GM02LC44512, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said GM02LC44512, or a functional equivalent or a homolog thereof as depicted in column 8 of Table II, application no. 6, preferably a homolog or functional equivalent as depicted in column 8 of Table II B, application no. 6, and being depicted in the same respective line as said GM02LC44512, and preferably the activity is increased non-targeted, whereby the respective line discloses in column 7 the fine chemical leucine.

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 46850, preferably the coding region thereof, conferred the production of or the increase in leucine compared with the wild type control.

The nucleic acid sequence of GM02LC44512 from Glycine max, e.g. as shown in the respective line in column 5 of Table I, application no. 6, is unpublished. And the activity of the gene product thereof is the activity of cyclin D.

Accordingly, in one embodiment, the process of the present invention for producing valine in a non-human organism, like a microorganism or a plant or a part thereof, comprises increasing or generating the activity of a gene product with the activity of a gene product conferring the activity of “cyclin D”, especially from Glycine max or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said GM02LC44512, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said GM02LC44512, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said GM02LC44512, or a functional equivalent or a homolog thereof as depicted in column 8 of Table II, application no. 6, preferably a homolog or functional equivalent as depicted in column 8 of Table II B, application no. 6, and being depicted in the same respective line as said GM02LC44512, and preferably the activity is increased non-targeted, whereby the respective line discloses in column 7 the fine chemical valine.

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 46850, preferably the coding region thereof, conferred the production of or the increase in valine compared with the wild type control.

The nucleic acid sequence of GM02LC5744 from Glycine max, e.g. as shown in the respective line in column 5 of Table I, application no. 6, is unpublished. And the activity of the gene product thereof is the activity of GM02LC5744-protein.

Accordingly, in one embodiment, the process of the present invention for producing leucine in a non-human organism, like a microorganism or a plant or a part thereof, comprises increasing or generating the activity of a gene product with the activity of a gene product conferring the activity of “GM02LC5744-protein”, especially from Glycine max or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said GM02LC5744, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said GM02LC5744, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said GM02LC5744, or a functional equivalent or a homolog thereof as depicted in column 8 of Table II, application no. 6, preferably a homolog or functional equivalent as depicted in column 8 of Table II B, application no. 6, and being depicted in the same respective line as said GM02LC5744, and preferably the activity is increased non-targeted, whereby the respective line discloses in column 7 the fine chemical leucine.

Accordingly, in one embodiment, the molecule which activity is to be increased in the process of the invention is the gene product with an activity as a “GM02LC5744-protein”, preferably it is the molecule of section (a) or (b) of this paragraph.

In particular, it was observed that in plants, especially in Arabidopsis thaliana, increasing or generating the activity of a gene product non-targeted with the activity of a “GM02LC5744-protein”, preferably being encoded by a gene comprising the nucleic acid sequence SEQ ID NO. 47076, preferably the coding region thereof, conferred the production of or the increase in leucine compared with the wild type control.

The nucleic acid sequence of Sll0228 from Synechocystis sp., e.g. as shown in the respective line in column 5 of Table I, application no. 6, has been published in C. And the activity of the gene product thereof is the activity of agmatinase.

Accordingly, in one embodiment, the process of the present invention for producing valine in a non-human organism, like a microorganism or a plant or a part thereof, comprises increasing or generating the activity of a gene product with the activity of a gene product conferring the activity of “agmatinase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said Sll0228, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Sll0228, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Sll0228, or a functional equivalent or a homolog thereof as depicted in column 8 of Table II, application no. 6, preferably a homolog or functional equivalent as depicted in column 8 of Table II B, application no. 6, and being depicted in the same respective line as said Sll0228, and preferably the activity is increased plastidic, whereby the respective line discloses in column 7 the fine chemical valine.

Accordingly, in one embodiment, the molecule which activity is to be increased in the process of the invention is the gene product with an activity as a “agmatinase”, preferably it is the molecule of section (a) or (b) of this paragraph.

In particular, it was observed that in plants, especially in Arabidopsis thaliana, increasing or generating the activity of a gene product plastidic with the activity of a “agmatinase”, preferably being encoded by a gene comprising the nucleic acid sequence SEQ ID NO. 84265, preferably the coding region thereof, conferred the production of or the increase in valine compared with the wild type control.

The nucleic acid sequence of Sll0240 from Synechocystis sp., e.g. as shown in the respective line in column 5 of Table I, application no. 6, has been published in C. And the activity of the gene product thereof is the activity of ABC transporter ATP binding component.

Accordingly, in one embodiment, the process of the present invention for producing valine in a non-human organism, like a microorganism or a plant or a part thereof, comprises increasing or generating the activity of a gene product with the activity of a gene product conferring the activity of “ABC transporter ATP binding component”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said Sll0240, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Sll0240, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Sll0240, or a functional equivalent or a homolog thereof as depicted in column 8 of Table II, application no. 6, preferably a homolog or functional equivalent as depicted in column 8 of Table II B, application no. 6, and being depicted in the same respective line as said Sll0240, and preferably the activity is increased plastidic, whereby the respective line discloses in column 7 the fine chemical valine.

Accordingly, in one embodiment, the molecule which activity is to be increased in the process of the invention is the gene product with an activity as a “ABC transporter ATP binding component”, preferably it is the molecule of section (a) or (b) of this paragraph.

In particular, it was observed that in plants, especially in Arabidopsis thaliana, increasing or generating the activity of a gene product plastidic with the activity of a “ABC transporter ATP binding component”, preferably being encoded by a gene comprising the nucleic acid sequence SEQ ID NO. 84441, preferably the coding region thereof, conferred the production of or the increase in valine compared with the wild type control.

The nucleic acid sequence of Sll0248 from Synechocystis sp., e.g. as shown in the respective line in column 5 of Table I, application no. 6, has been published in C. And the activity of the gene product thereof is the activity of flavodoxin.

Accordingly, in one embodiment, the process of the present invention for producing isoleucine in a non-human organism, like a microorganism or a plant or a part thereof, comprises increasing or generating the activity of a gene product with the activity of a gene product conferring the activity of “flavodoxin”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Sll0248, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Sll0248, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Sll0248, or a functional equivalent or a homolog thereof as depicted in column 8 of Table II, application no. 6, preferably a homolog or functional equivalent as depicted in column 8 of Table II B, application no. 6, and being depicted in the same respective line as said Sll0248, and preferably the activity is increased plastidic, whereby the respective line discloses in column 7 the fine chemical isoleucine.

Accordingly, in one embodiment, the molecule which activity is to be increased in the process of the invention is the gene product with an activity as a “flavodoxin”, preferably it is the molecule of section (a) or (b) of this paragraph.

In particular, it was observed that in plants, especially in Arabidopsis thaliana, increasing or generating the activity of a gene product plastidic with the activity of a “flavodoxin”, preferably being encoded by a gene comprising the nucleic acid sequence SEQ ID NO. 49143, preferably the coding region thereof, conferred the production of or the increase in isoleucine compared with the wild type control.

The nucleic acid sequence of Sll0248 from Synechocystis sp., e.g. as shown in the respective line in column 5 of Table I, application no. 6, has been published in C. And the activity of the gene product thereof is the activity of flavodoxin.

Accordingly, in one embodiment, the process of the present invention for producing leucine in a non-human organism, like a microorganism or a plant or a part thereof, comprises increasing or generating the activity of a gene product with the activity of a gene product conferring the activity of “flavodoxin”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said Sll0248, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Sll0248, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Sll0248, or a functional equivalent or a homolog thereof as depicted in column 8 of Table II, application no. 6, preferably a homolog or functional equivalent as depicted in column 8 of Table II B, application no. 6, and being depicted in the same respective line as said Sll0248, and preferably the activity is increased plastidic, whereby the respective line discloses in column 7 the fine chemical leucine.

Accordingly, in one embodiment, the molecule which activity is to be increased in the process of the invention is the gene product with an activity as a “flavodoxin”, preferably it is the molecule of section (a) or (b) of this paragraph.

In particular, it was observed that in plants, especially in Arabidopsis thaliana, increasing or generating the activity of a gene product plastidic with the activity of a “flavodoxin”, preferably being encoded by a gene comprising the nucleic acid sequence SEQ ID NO. 49143, preferably the coding region thereof, conferred the production of or the increase in leucine compared with the wild type control.

The nucleic acid sequence of Sll0290 from Synechocystis sp., e.g. as shown in the respective line in column 5 of Table I, application no. 6, has been published in C. And the activity of the gene product thereof is the activity of polyphosphate kinase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “polyphosphate kinase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Sll0290, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Sll0290, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Sll0290, or a functional equivalent or a homolog thereof as depicted in column 8 of Table II, application no. 6, preferably a homolog or functional equivalent as depicted in column 8 of Table II B, application no. 6, and being depicted in the same respective line as said Sll0290, and preferably the activity is increased non-targeted, whereby the respective line discloses in column 7 the fine chemical isoleucine.

Accordingly, in one embodiment, the molecule which activity is to be increased in the process of the invention is the gene product with an activity as a “polyphosphate kinase”, preferably it is the molecule of section (a) or (b) of this paragraph.

In particular, it was observed that in plants, especially in Arabidopsis thaliana, increasing or generating the activity of a gene product non-targeted with the activity of a “polyphosphate kinase”, preferably being encoded by a gene comprising the nucleic acid sequence SEQ ID NO. 49828, preferably the coding region thereof, conferred the production of or the increase in isoleucine compared with the wild type control.

The nucleic acid sequence of Sll0290 from Synechocystis sp., e.g. as shown in the respective line in column 5 of Table I, application no. 6, has been published in C. And the activity of the gene product thereof is the activity of polyphosphate kinase.

Accordingly, in one embodiment, the process of the present invention for producing leucine in a non-human organism, like a microorganism or a plant or a part thereof, comprises increasing or generating the activity of a gene product with the activity of a gene product conferring the activity of “polyphosphate kinase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said Sll0290, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Sll0290, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Sll0290, or a functional equivalent or a homolog thereof as depicted in column 8 of Table II, application no. 6, preferably a homolog or functional equivalent as depicted in column 8 of Table II B, application no. 6, and being depicted in the same respective line as said Sll0290, and preferably the activity is increased non-targeted, whereby the respective line discloses in column 7 the fine chemical leucine.

Accordingly, in one embodiment, the molecule which activity is to be increased in the process of the invention is the gene product with an activity as a “polyphosphate kinase”, preferably it is the molecule of section (a) or (b) of this paragraph.

In particular, it was observed that in plants, especially in Arabidopsis thaliana, increasing or generating the activity of a gene product non-targeted with the activity of a “polyphosphate kinase”, preferably being encoded by a gene comprising the nucleic acid sequence SEQ ID NO. 49828, preferably the coding region thereof, conferred the production of or the increase in leucine compared with the wild type control.

The nucleic acid sequence of Sll0290 from Synechocystis sp., e.g. as shown in the respective line in column 5 of Table I, application no. 6, has been published in C. And the activity of the gene product thereof is the activity of polyphosphate kinase.

Accordingly, in one embodiment, the process of the present invention for producing valine in a non-human organism, like a microorganism or a plant or a part thereof, comprises increasing or generating the activity of a gene product with the activity of a gene product conferring the activity of “polyphosphate kinase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said Sll0290, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Sll0290, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Sll0290, or a functional equivalent or a homolog thereof as depicted in column 8 of Table II, application no. 6, preferably a homolog or functional equivalent as depicted in column 8 of Table II B, application no. 6, and being depicted in the same respective line as said Sll0290, and preferably the activity is increased non-targeted, whereby the respective line discloses in column 7 the fine chemical valine.

Accordingly, in one embodiment, the molecule which activity is to be increased in the process of the invention is the gene product with an activity as a “polyphosphate kinase”, preferably it is the molecule of section (a) or (b) of this paragraph.

In particular, it was observed that in plants, especially in Arabidopsis thaliana, increasing or generating the activity of a gene product non-targeted with the activity of a “polyphosphate kinase”, preferably being encoded by a gene comprising the nucleic acid sequence SEQ ID NO. 49828, preferably the coding region thereof, conferred the production of or the increase in valine compared with the wild type control.

The nucleic acid sequence of Sll0368 from Synechocystis sp., e.g. as shown in the respective line in column 5 of Table I, application no. 6, has been published in C. And the activity of the gene product thereof is the activity of bifunctional protein (phosphoribosyltransferase and regulatory protein).

Accordingly, in one embodiment, the process of the present invention for producing valine in a non-human organism, like a microorganism or a plant or a part thereof, comprises increasing or generating the activity of a gene product with the activity of a gene product conferring the activity of “bifunctional protein (phosphoribosyltransferase and regulatory protein)”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said Sll0368, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Sll0368, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Sll0368, or a functional equivalent or a homolog thereof as depicted in column 8 of Table II, application no. 6, preferably a homolog or functional equivalent as depicted in column 8 of Table II B, application no. 6, and being depicted in the same respective line as said Sll0368, and preferably the activity is increased mitochondrial, whereby the respective line discloses in column 7 the fine chemical valine.

Accordingly, in one embodiment, the molecule which activity is to be increased in the process of the invention is the gene product with an activity as a “bifunctional protein (phosphoribosyltransferase and regulatory protein)”, preferably it is the molecule of section (a) or (b) of this paragraph.

In particular, it was observed that in plants, especially in Arabidopsis thaliana, increasing or generating the activity of a gene product mitochondrial with the activity of a “bifunctional protein (phosphoribosyltransferase and regulatory protein)”, preferably being encoded by a gene comprising the nucleic acid sequence SEQ ID NO. 106920, preferably the coding region thereof, conferred the production of or the increase in valine compared with the wild type control.

The nucleic acid sequence of Sll0420 from Synechocystis sp., e.g. as shown in the respective line in column 5 of Table I, application no. 6, has been published in C. And the activity of the gene product thereof is the activity of urease subunit.

Accordingly, in one embodiment, the process of the present invention for producing isoleucine in a non-human organism, like a microorganism or a plant or a part thereof, comprises increasing or generating the activity of a gene product with the activity of a gene product conferring the activity of “urease subunit”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Sll0420, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Sll0420, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Sll0420, or a functional equivalent or a homolog thereof as depicted in column 8 of Table II, application no. 6, preferably a homolog or functional equivalent as depicted in column 8 of Table II B, application no. 6, and being depicted in the same respective line as said Sll0420, and preferably the activity is increased non-targeted, whereby the respective line discloses in column 7 the fine chemical isoleucine.

Accordingly, in one embodiment, the molecule which activity is to be increased in the process of the invention is the gene product with an activity as a “urease subunit”, preferably it is the molecule of section (a) or (b) of this paragraph.

In particular, it was observed that in plants, especially in Arabidopsis thaliana, increasing or generating the activity of a gene product non-targeted with the activity of a “urease subunit”, preferably being encoded by a gene comprising the nucleic acid sequence SEQ ID NO. 50104, preferably the coding region thereof, conferred the production of or the increase in isoleucine compared with the wild type control.

The nucleic acid sequence of Sll0521 from Synechocystis sp., e.g. as shown in the respective line in column 5 of Table I, application no. 6, has been published in C. And the activity of the gene product thereof is the activity of NAD(P)H-quinone oxidoreductase subunit.

Accordingly, in one embodiment, the process of the present invention for producing valine in a non-human organism, like a microorganism or a plant or a part thereof, comprises increasing or generating the activity of a gene product with the activity of a gene product conferring the activity of “NAD(P)H-quinone oxidoreductase subunit”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said Sll0521, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Sll0521, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Sll0521, or a functional equivalent or a homolog thereof as depicted in column 8 of Table II, application no. 6, preferably a homolog or functional equivalent as depicted in column 8 of Table II B, application no. 6, and being depicted in the same respective line as said Sll0521, and preferably the activity is increased plastidic, whereby the respective line discloses in column 7 the fine chemical valine.

Accordingly, in one embodiment, the molecule which activity is to be increased in the process of the invention is the gene product with an activity as a “NAD(P)H-quinone oxidoreductase subunit”, preferably it is the molecule of section (a) or (b) of this paragraph.

In particular, it was observed that in plants, especially in Arabidopsis thaliana, increasing or generating the activity of a gene product plastidic with the activity of a “NAD(P)H-quinone oxidoreductase subunit”, preferably being encoded by a gene comprising the nucleic acid sequence SEQ ID NO. 50339, preferably the coding region thereof, conferred the production of or the increase in valine compared with the wild type control.

The nucleic acid sequence of Sll0816 from Synechocystis sp., e.g. as shown in the respective line in column 5 of Table I, application no. 6, has been published in C. And the activity of the gene product thereof is the activity of oxireductase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “oxireductase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Sll0816, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Sll0816, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Sll0816, or a functional equivalent or a homolog thereof as depicted in column 8 of Table II, application no. 6, preferably a homolog or functional equivalent as depicted in column 8 of Table II B, application no. 6, and being depicted in the same respective line as said Sll0816, and preferably the activity is increased non-targeted, whereby the respective line discloses in column 7 the fine chemical isoleucine.

Accordingly, in one embodiment, the molecule which activity is to be increased in the process of the invention is the gene product with an activity as a “oxireductase”, preferably it is the molecule of section (a) or (b) of this paragraph.

In particular, it was observed that in plants, especially in Arabidopsis thaliana, increasing or generating the activity of a gene product non-targeted with the activity of a “oxireductase”, preferably being encoded by a gene comprising the nucleic acid sequence SEQ ID NO. 51198, preferably the coding region thereof, conferred the production of or the increase in isoleucine compared with the wild type control.

The nucleic acid sequence of Sll0833 from Synechocystis sp., e.g. as shown in the respective line in column 5 of Table I, application no. 6, has been published in C. And the activity of the gene product thereof is the activity of binding-protein-dependent transport systems inner membrane component.

Accordingly, in one embodiment, the process of the present invention for producing isoleucine in a non-human organism, like a microorganism or a plant or a part thereof, comprises increasing or generating the activity of a gene product with the activity of a gene product conferring the activity of “binding-protein-dependent transport systems inner membrane component”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Sll0833, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Sll0833, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Sll0833, or a functional equivalent or a homolog thereof as depicted in column 8 of Table II, application no. 6, preferably a homolog or functional equivalent as depicted in column 8 of Table II B, application no. 6, and being depicted in the same respective line as said Sll0833, and preferably the activity is increased non-targeted, whereby the respective line discloses in column 7 the fine chemical isoleucine.

Accordingly, in one embodiment, the molecule which activity is to be increased in the process of the invention is the gene product with an activity as a “binding-protein-dependent transport systems inner membrane component”, preferably it is the molecule of section (a) or (b) of this paragraph.

In particular, it was observed that in plants, especially in Arabidopsis thaliana, increasing or generating the activity of a gene product non-targeted with the activity of a “binding-protein-dependent transport systems inner membrane component”, preferably being encoded by a gene comprising the nucleic acid sequence SEQ ID NO. 101707, preferably the coding region thereof, conferred the production of or the increase in isoleucine compared with the wild type control.

The nucleic acid sequence of Sll1032 from Synechocystis sp., e.g. as shown in the respective line in column 5 of Table I, application no. 6, has been published in C. And the activity of the gene product thereof is the activity of sll1032-protein.

Accordingly, in one embodiment, the process of the present invention for producing valine in a non-human organism, like a microorganism or a plant or a part thereof, comprises increasing or generating the activity of a gene product with the activity of a gene product conferring the activity of “sll1032-protein”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said Sll1032, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Sll1032, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Sll1032, or a functional equivalent or a homolog thereof as depicted in column 8 of Table II, application no. 6, preferably a homolog or functional equivalent as depicted in column 8 of Table II B, application no. 6, and being depicted in the same respective line as said Sll1032, and preferably the activity is increased mitochondrial, whereby the respective line discloses in column 7 the fine chemical valine.

Accordingly, in one embodiment, the molecule which activity is to be increased in the process of the invention is the gene product with an activity as a “sll1032-protein”, preferably it is the molecule of section (a) or (b) of this paragraph.

In particular, it was observed that in plants, especially in Arabidopsis thaliana, increasing or generating the activity of a gene product mitochondrial with the activity of a “sll1032-protein”, preferably being encoded by a gene comprising the nucleic acid sequence SEQ ID NO. 85723, preferably the coding region thereof, conferred the production of or the increase in valine compared with the wild type control.

The nucleic acid sequence of Sll1393 from Synechocystis sp., e.g. as shown in the respective line in column 5 of Table I, application no. 6, has been published in C. And the activity of the gene product thereof is the activity of glycogen (starch) synthase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “glycogen (starch) synthase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Sll1393, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Sll1393, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Sll1393, or a functional equivalent or a homolog thereof as depicted in column 8 of Table II, application no. 6, preferably a homolog or functional equivalent as depicted in column 8 of Table II B, application no. 6, and being depicted in the same respective line as said Sll1393, and preferably the activity is increased non-targeted, whereby the respective line discloses in column 7 the fine chemical isoleucine.

Accordingly, in one embodiment, the molecule which activity is to be increased in the process of the invention is the gene product with an activity as a “glycogen (starch) synthase”, preferably it is the molecule of section (a) or (b) of this paragraph.

In particular, it was observed that in plants, especially in Arabidopsis thaliana, increasing or generating the activity of a gene product non-targeted with the activity of a “glycogen (starch) synthase”, preferably being encoded by a gene comprising the nucleic acid sequence SEQ ID NO. 53456, preferably the coding region thereof, conferred the production of or the increase in isoleucine compared with the wild type control.

The nucleic acid sequence of Sll1393 from Synechocystis sp., e.g. as shown in the respective line in column 5 of Table I, application no. 6, has been published in C. And the activity of the gene product thereof is the activity of glycogen (starch) synthase.

Accordingly, in one embodiment, the process of the present invention for producing leucine in a non-human organism, like a microorganism or a plant or a part thereof, comprises increasing or generating the activity of a gene product with the activity of a gene product conferring the activity of “glycogen (starch) synthase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said Sll1393, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Sll1393, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Sll1393, or a functional equivalent or a homolog thereof as depicted in column 8 of Table II, application no. 6, preferably a homolog or functional equivalent as depicted in column 8 of Table II B, application no. 6, and being depicted in the same respective line as said Sll1393, and preferably the activity is increased non-targeted, whereby the respective line discloses in column 7 the fine chemical leucine.

Accordingly, in one embodiment, the molecule which activity is to be increased in the process of the invention is the gene product with an activity as a “glycogen (starch) synthase”, preferably it is the molecule of section (a) or (b) of this paragraph.

In particular, it was observed that in plants, especially in Arabidopsis thaliana, increasing or generating the activity of a gene product non-targeted with the activity of a “glycogen (starch) synthase”, preferably being encoded by a gene comprising the nucleic acid sequence SEQ ID NO. 53456, preferably the coding region thereof, conferred the production of or the increase in leucine compared with the wild type control.

The nucleic acid sequence of Sll1393 from Synechocystis sp., e.g. as shown in the respective line in column 5 of Table I, application no. 6, has been published in C. And the activity of the gene product thereof is the activity of glycogen (starch) synthase.

Accordingly, in one embodiment, the process of the present invention for producing valine in a non-human organism, like a microorganism or a plant or a part thereof, comprises increasing or generating the activity of a gene product with the activity of a gene product conferring the activity of “glycogen (starch) synthase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said Sll1393, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Sll1393, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Sll1393, or a functional equivalent or a homolog thereof as depicted in column 8 of Table II, application no. 6, preferably a homolog or functional equivalent as depicted in column 8 of Table II B, application no. 6, and being depicted in the same respective line as said Sll1393, and preferably the activity is increased non-targeted, whereby the respective line discloses in column 7 the fine chemical valine.

Accordingly, in one embodiment, the molecule which activity is to be increased in the process of the invention is the gene product with an activity as a “glycogen (starch) synthase”, preferably it is the molecule of section (a) or (b) of this paragraph.

In particular, it was observed that in plants, especially in Arabidopsis thaliana, increasing or generating the activity of a gene product non-targeted with the activity of a “glycogen (starch) synthase”, preferably being encoded by a gene comprising the nucleic acid sequence SEQ ID NO. 53456, preferably the coding region thereof, conferred the production of or the increase in valine compared with the wild type control.

The nucleic acid sequence of Sll1455 from Synechocystis sp., e.g. as shown in the respective line in column 5 of Table I, application no. 6, has been published in C. And the activity of the gene product thereof is the activity of sll1455-protein.

Accordingly, in one embodiment, the process of the present invention for producing valine in a non-human organism, like a microorganism or a plant or a part thereof, comprises increasing or generating the activity of a gene product with the activity of a gene product conferring the activity of “sll1455-protein”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said Sll1455, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Sll1455, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Sll1455, or a functional equivalent or a homolog thereof as depicted in column 8 of Table II, application no. 6, preferably a homolog or functional equivalent as depicted in column 8 of Table II B, application no. 6, and being depicted in the same respective line as said Sll1455, and preferably the activity is increased plastidic, whereby the respective line discloses in column 7 the fine chemical valine.

Accordingly, in one embodiment, the molecule which activity is to be increased in the process of the invention is the gene product with an activity as a “sll1455-protein”, preferably it is the molecule of section (a) or (b) of this paragraph.

In particular, it was observed that in plants, especially in Arabidopsis thaliana, increasing or generating the activity of a gene product plastidic with the activity of a “sll1455-protein”, preferably being encoded by a gene comprising the nucleic acid sequence SEQ ID NO. 107100, preferably the coding region thereof, conferred the production of or the increase in valine compared with the wild type control.

The nucleic acid sequence of Sll1498 from Synechocystis sp., e.g. as shown in the respective line in column 5 of Table I, application no. 6, has been published in C. And the activity of the gene product thereof is the activity of carbamoyl-phosphate synthase subunit.

Accordingly, in one embodiment, the process of the present invention for producing isoleucine in a non-human organism, like a microorganism or a plant or a part thereof, comprises increasing or generating the activity of a gene product with the activity of a gene product conferring the activity of “carbamoyl-phosphate synthase subunit”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Sll1498, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Sll1498, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Sll1498, or a functional equivalent or a homolog thereof as depicted in column 8 of Table II, application no. 6, preferably a homolog or functional equivalent as depicted in column 8 of Table II B, application no. 6, and being depicted in the same respective line as said Sll1498, and preferably the activity is increased plastidic, whereby the respective line discloses in column 7 the fine chemical isoleucine.

Accordingly, in one embodiment, the molecule which activity is to be increased in the process of the invention is the gene product with an activity as a “carbamoyl-phosphate synthase subunit”, preferably it is the molecule of section (a) or (b) of this paragraph.

In particular, it was observed that in plants, especially in Arabidopsis thaliana, increasing or generating the activity of a gene product plastidic with the activity of a “carbamoyl-phosphate synthase subunit”, preferably being encoded by a gene comprising the nucleic acid sequence SEQ ID NO. 95047, preferably the coding region thereof, conferred the production of or the increase in isoleucine compared with the wild type control.

The nucleic acid sequence of Sll1498 from Synechocystis sp., e.g. as shown in the respective line in column 5 of Table I, application no. 6, has been published in C. And the activity of the gene product thereof is the activity of carbamoyl-phosphate synthase subunit.

Accordingly, in one embodiment, the process of the present invention for producing leucine in a non-human organism, like a microorganism or a plant or a part thereof, comprises increasing or generating the activity of a gene product with the activity of a gene product conferring the activity of “carbamoyl-phosphate synthase subunit”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said Sll1498, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Sll1498, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Sll1498, or a functional equivalent or a homolog thereof as depicted in column 8 of Table II, application no. 6, preferably a homolog or functional equivalent as depicted in column 8 of Table II B, application no. 6, and being depicted in the same respective line as said Sll1498, and preferably the activity is increased plastidic, whereby the respective line discloses in column 7 the fine chemical leucine.

Accordingly, in one embodiment, the molecule which activity is to be increased in the process of the invention is the gene product with an activity as a “carbamoyl-phosphate synthase subunit”, preferably it is the molecule of section (a) or (b) of this paragraph.

In particular, it was observed that in plants, especially in Arabidopsis thaliana, increasing or generating the activity of a gene product plastidic with the activity of a “carbamoyl-phosphate synthase subunit”, preferably being encoded by a gene comprising the nucleic acid sequence SEQ ID NO. 95047, preferably the coding region thereof, conferred the production of or the increase in leucine compared with the wild type control.

The nucleic acid sequence of Sll1761 from Synechocystis sp., e.g. as shown in the respective line in column 5 of Table I, application no. 6, has been published in C. And the activity of the gene product thereof is the activity of sll1761-protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “sll1761-protein”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Sll1761, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Sll1761, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Sll1761, or a functional equivalent or a homolog thereof as depicted in column 8 of Table II, application no. 6, preferably a homolog or functional equivalent as depicted in column 8 of Table II B, application no. 6, and being depicted in the same respective line as said Sll1761, and preferably the activity is increased non-targeted, whereby the respective line discloses in column 7 the fine chemical isoleucine.

Accordingly, in one embodiment, the molecule which activity is to be increased in the process of the invention is the gene product with an activity as a “sll1761-protein”, preferably it is the molecule of section (a) or (b) of this paragraph.

In particular, it was observed that in plants, especially in Arabidopsis thaliana, increasing or generating the activity of a gene product non-targeted with the activity of a “sll1761-protein”, preferably being encoded by a gene comprising the nucleic acid sequence SEQ ID NO. 55379, preferably the coding region thereof, conferred the production of or the increase in isoleucine compared with the wild type control.

The nucleic acid sequence of Sll1761 from Synechocystis sp., e.g. as shown in the respective line in column 5 of Table I, application no. 6, has been published in C. And the activity of the gene product thereof is the activity of sll1761-protein.

Accordingly, in one embodiment, the process of the present invention for producing leucine in a non-human organism, like a microorganism or a plant or a part thereof, comprises increasing or generating the activity of a gene product with the activity of a gene product conferring the activity of “sll1761-protein”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said Sll1761, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Sll1761, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Sll1761, or a functional equivalent or a homolog thereof as depicted in column 8 of Table II, application no. 6, preferably a homolog or functional equivalent as depicted in column 8 of Table II B, application no. 6, and being depicted in the same respective line as said Sll1761, and preferably the activity is increased non-targeted, whereby the respective line discloses in column 7 the fine chemical leucine.

Accordingly, in one embodiment, the molecule which activity is to be increased in the process of the invention is the gene product with an activity as a “sll1761-protein”, preferably it is the molecule of section (a) or (b) of this paragraph.

In particular, it was observed that in plants, especially in Arabidopsis thaliana, increasing or generating the activity of a gene product non-targeted with the activity of a “sll1761-protein”, preferably being encoded by a gene comprising the nucleic acid sequence SEQ ID NO. 55379, preferably the coding region thereof, conferred the production of or the increase in leucine compared with the wild type control.

The nucleic acid sequence of Sll1815 from Synechocystis sp., e.g. as shown in the respective line in column 5 of Table I, application no. 6, has been published in C. And the activity of the gene product thereof is the activity of adenylate kinase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “adenylate kinase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Sll1815, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Sll1815, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Sll1815, or a functional equivalent or a homolog thereof as depicted in column 8 of Table II, application no. 6, preferably a homolog or functional equivalent as depicted in column 8 of Table II B, application no. 6, and being depicted in the same respective line as said Sll1815, and preferably the activity is increased mitochondrial, whereby the respective line discloses in column 7 the fine chemical isoleucine.

Accordingly, in one embodiment, the molecule which activity is to be increased in the process of the invention is the gene product with an activity as a “adenylate kinase”, preferably it is the molecule of section (a) or (b) of this paragraph.

In particular, it was observed that in plants, especially in Arabidopsis thaliana, increasing or generating the activity of a gene product mitochondrial with the activity of a “adenylate kinase”, preferably being encoded by a gene comprising the nucleic acid sequence SEQ ID NO. 85808, preferably the coding region thereof, conferred the production of or the increase in isoleucine compared with the wild type control.

The nucleic acid sequence of Sll1815 from Synechocystis sp., e.g. as shown in the respective line in column 5 of Table I, application no. 6, has been published in C. And the activity of the gene product thereof is the activity of adenylate kinase.

Accordingly, in one embodiment, the process of the present invention for producing leucine in a non-human organism, like a microorganism or a plant or a part thereof, comprises increasing or generating the activity of a gene product with the activity of a gene product conferring the activity of “adenylate kinase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said Sll1815, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Sll1815, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Sll1815, or a functional equivalent or a homolog thereof as depicted in column 8 of Table II, application no. 6, preferably a homolog or functional equivalent as depicted in column 8 of Table II B, application no. 6, and being depicted in the same respective line as said Sll1815, and preferably the activity is increased mitochondrial, whereby the respective line discloses in column 7 the fine chemical leucine.

Accordingly, in one embodiment, the molecule which activity is to be increased in the process of the invention is the gene product with an activity as a “adenylate kinase”, preferably it is the molecule of section (a) or (b) of this paragraph.

In particular, it was observed that in plants, especially in Arabidopsis thaliana, increasing or generating the activity of a gene product mitochondrial with the activity of a “adenylate kinase”, preferably being encoded by a gene comprising the nucleic acid sequence SEQ ID NO. 85808, preferably the coding region thereof, conferred the production of or the increase in leucine compared with the wild type control.

The nucleic acid sequence of Sll1815 from Synechocystis sp., e.g. as shown in the respective line in column 5 of Table I, application no. 6, has been published in C. And the activity of the gene product thereof is the activity of adenylate kinase.

Accordingly, in one embodiment, the process of the present invention for producing valine in a non-human organism, like a microorganism or a plant or a part thereof, comprises increasing or generating the activity of a gene product with the activity of a gene product conferring the activity of “adenylate kinase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said Sll1815, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Sll1815, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Sll1815, or a functional equivalent or a homolog thereof as depicted in column 8 of Table II, application no. 6, preferably a homolog or functional equivalent as depicted in column 8 of Table II B, application no. 6, and being depicted in the same respective line as said Sll1815, and preferably the activity is increased mitochondrial, whereby the respective line discloses in column 7 the fine chemical valine.

Accordingly, in one embodiment, the molecule which activity is to be increased in the process of the invention is the gene product with an activity as a “adenylate kinase”, preferably it is the molecule of section (a) or (b) of this paragraph.

In particular, it was observed that in plants, especially in Arabidopsis thaliana, increasing or generating the activity of a gene product mitochondrial with the activity of a “adenylate kinase”, preferably being encoded by a gene comprising the nucleic acid sequence SEQ ID NO. 85808, preferably the coding region thereof, conferred the production of or the increase in valine compared with the wild type control.

The nucleic acid sequence of Sll1848 from Synechocystis sp., e.g. as shown in the respective line in column 5 of Table I, application no. 6, has been published in C. And the activity of the gene product thereof is the activity of acyl transferase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “acyl transferase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Sll1848, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Sll1848, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Sll1848, or a functional equivalent or a homolog thereof as depicted in column 8 of Table II, application no. 6, preferably a homolog or functional equivalent as depicted in column 8 of Table II B, application no. 6, and being depicted in the same respective line as said Sll1848, and preferably the activity is increased plastidic, whereby the respective line discloses in column 7 the fine chemical isoleucine.

Accordingly, in one embodiment, the molecule which activity is to be increased in the process of the invention is the gene product with an activity as a “acyl transferase”, preferably it is the molecule of section (a) or (b) of this paragraph.

In particular, it was observed that in plants, especially in Arabidopsis thaliana, increasing or generating the activity of a gene product plastidic with the activity of a “acyl transferase”, preferably being encoded by a gene comprising the nucleic acid sequence SEQ ID NO. 86447, preferably the coding region thereof, conferred the production of or the increase in isoleucine compared with the wild type control.

The nucleic acid sequence of Slr0150 from Synechocystis sp., e.g. as shown in the respective line in column 5 of Table I, application no. 6, has been published in C. And the activity of the gene product thereof is the activity of ferredoxin.

Accordingly, in one embodiment, the process of the present invention for producing valine in a non-human organism, like a microorganism or a plant or a part thereof, comprises increasing or generating the activity of a gene product with the activity of a gene product conferring the activity of “ferredoxin”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said Slr0150, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr0150, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr0150, or a functional equivalent or a homolog thereof as depicted in column 8 of Table II, application no. 6, preferably a homolog or functional equivalent as depicted in column 8 of Table II B, application no. 6, and being depicted in the same respective line as said Slr0150, and preferably the activity is increased mitochondrial, whereby the respective line discloses in column 7 the fine chemical valine.

Accordingly, in one embodiment, the molecule which activity is to be increased in the process of the invention is the gene product with an activity as a “ferredoxin”, preferably it is the molecule of section (a) or (b) of this paragraph.

In particular, it was observed that in plants, especially in Arabidopsis thaliana, increasing or generating the activity of a gene product mitochondrial with the activity of a “ferredoxin”, preferably being encoded by a gene comprising the nucleic acid sequence SEQ ID NO. 95527, preferably the coding region thereof, conferred the production of or the increase in valine compared with the wild type control.

The nucleic acid sequence of Slr0239 from Synechocystis sp., e.g. as shown in the respective line in column 5 of Table I, application no. 6, has been published in C. And the activity of the gene product thereof is the activity of precorrin methylase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “precorrin methylase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Slr0239, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr0239, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr0239, or a functional equivalent or a homolog thereof as depicted in column 8 of Table II, application no. 6, preferably a homolog or functional equivalent as depicted in column 8 of Table II B, application no. 6, and being depicted in the same respective line as said Slr0239, and preferably the activity is increased mitochondrial, whereby the respective line discloses in column 7 the fine chemical isoleucine.

Accordingly, in one embodiment, the molecule which activity is to be increased in the process of the invention is the gene product with an activity as a “precorrin methylase”, preferably it is the molecule of section (a) or (b) of this paragraph.

In particular, it was observed that in plants, especially in Arabidopsis thaliana, increasing or generating the activity of a gene product mitochondrial with the activity of a “precorrin methylase”, preferably being encoded by a gene comprising the nucleic acid sequence SEQ ID NO. 86540, preferably the coding region thereof, conferred the production of or the increase in isoleucine compared with the wild type control.

The nucleic acid sequence of Slr0239 from Synechocystis sp., e.g. as shown in the respective line in column 5 of Table I, application no. 6, has been published in C. And the activity of the gene product thereof is the activity of precorrin methylase.

Accordingly, in one embodiment, the process of the present invention for producing leucine in a non-human organism, like a microorganism or a plant or a part thereof, comprises increasing or generating the activity of a gene product with the activity of a gene product conferring the activity of “precorrin methylase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said Slr0239, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr0239, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr0239, or a functional equivalent or a homolog thereof as depicted in column 8 of Table II, application no. 6, preferably a homolog or functional equivalent as depicted in column 8 of Table II B, application no. 6, and being depicted in the same respective line as said Slr0239, and preferably the activity is increased mitochondrial, whereby the respective line discloses in column 7 the fine chemical leucine.

Accordingly, in one embodiment, the molecule which activity is to be increased in the process of the invention is the gene product with an activity as a “precorrin methylase”, preferably it is the molecule of section (a) or (b) of this paragraph.

In particular, it was observed that in plants, especially in Arabidopsis thaliana, increasing or generating the activity of a gene product mitochondrial with the activity of a “precorrin methylase”, preferably being encoded by a gene comprising the nucleic acid sequence SEQ ID NO. 86540, preferably the coding region thereof, conferred the production of or the increase in leucine compared with the wild type control.

The nucleic acid sequence of Slr0239 from Synechocystis sp., e.g. as shown in the respective line in column 5 of Table I, application no. 6, has been published in C. And the activity of the gene product thereof is the activity of precorrin methylase.

Accordingly, in one embodiment, the process of the present invention for producing valine in a non-human organism, like a microorganism or a plant or a part thereof, comprises increasing or generating the activity of a gene product with the activity of a gene product conferring the activity of “precorrin methylase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said Slr0239, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr0239, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr0239, or a functional equivalent or a homolog thereof as depicted in column 8 of Table II, application no. 6, preferably a homolog or functional equivalent as depicted in column 8 of Table II B, application no. 6, and being depicted in the same respective line as said Slr0239, and preferably the activity is increased mitochondrial, whereby the respective line discloses in column 7 the fine chemical valine.

Accordingly, in one embodiment, the molecule which activity is to be increased in the process of the invention is the gene product with an activity as a “precorrin methylase”, preferably it is the molecule of section (a) or (b) of this paragraph.

In particular, it was observed that in plants, especially in Arabidopsis thaliana, increasing or generating the activity of a gene product mitochondrial with the activity of a “precorrin methylase”, preferably being encoded by a gene comprising the nucleic acid sequence SEQ ID NO. 86540, preferably the coding region thereof, conferred the production of or the increase in valine compared with the wild type control.

The nucleic acid sequence of Slr0721 from Synechocystis sp., e.g. as shown in the respective line in column 5 of Table I, application no. 6, has been published in C. And the activity of the gene product thereof is the activity of malate dehydrogenase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “malate dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Slr0721, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr0721, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr0721, or a functional equivalent or a homolog thereof as depicted in column 8 of Table II, application no. 6, preferably a homolog or functional equivalent as depicted in column 8 of Table II B, application no. 6, and being depicted in the same respective line as said Slr0721, and preferably the activity is increased non-targeted, whereby the respective line discloses in column 7 the fine chemical isoleucine.

Accordingly, in one embodiment, the molecule which activity is to be increased in the process of the invention is the gene product with an activity as a “malate dehydrogenase”, preferably it is the molecule of section (a) or (b) of this paragraph.

In particular, it was observed that in plants, especially in Arabidopsis thaliana, increasing or generating the activity of a gene product non-targeted with the activity of a “malate dehydrogenase”, preferably being encoded by a gene comprising the nucleic acid sequence SEQ ID NO. 87397, preferably the coding region thereof, conferred the production of or the increase in isoleucine compared with the wild type control.

The nucleic acid sequence of Slr0721 from Synechocystis sp., e.g. as shown in the respective line in column 5 of Table I, application no. 6, has been published in C. And the activity of the gene product thereof is the activity of malate dehydrogenase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “malate dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Slr0721, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr0721, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr0721, or a functional equivalent or a homolog thereof as depicted in column 8 of Table II, application no. 6, preferably a homolog or functional equivalent as depicted in column 8 of Table II B, application no. 6, and being depicted in the same respective line as said Slr0721, and preferably the activity is increased plastidic, whereby the respective line discloses in column 7 the fine chemical isoleucine.

Accordingly, in one embodiment, the molecule which activity is to be increased in the process of the invention is the gene product with an activity as a “malate dehydrogenase”, preferably it is the molecule of section (a) or (b) of this paragraph.

In particular, it was observed that in plants, especially in Arabidopsis thaliana, increasing or generating the activity of a gene product plastidic with the activity of a “malate dehydrogenase”, preferably being encoded by a gene comprising the nucleic acid sequence SEQ ID NO. 87397, preferably the coding region thereof, conferred the production of or the increase in isoleucine compared with the wild type control.

The nucleic acid sequence of Slr0721 from Synechocystis sp., e.g. as shown in the respective line in column 5 of Table I, application no. 6, has been published in C. And the activity of the gene product thereof is the activity of malate dehydrogenase.

Accordingly, in one embodiment, the process of the present invention for producing leucine in a non-human organism, like a microorganism or a plant or a part thereof, comprises increasing or generating the activity of a gene product with the activity of a gene product conferring the activity of “malate dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said Slr0721, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr0721, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr0721, or a functional equivalent or a homolog thereof as depicted in column 8 of Table II, application no. 6, preferably a homolog or functional equivalent as depicted in column 8 of Table II B, application no. 6, and being depicted in the same respective line as said Slr0721, and preferably the activity is increased plastidic, whereby the respective line discloses in column 7 the fine chemical leucine.

Accordingly, in one embodiment, the molecule which activity is to be increased in the process of the invention is the gene product with an activity as a “malate dehydrogenase”, preferably it is the molecule of section (a) or (b) of this paragraph.

In particular, it was observed that in plants, especially in Arabidopsis thaliana, increasing or generating the activity of a gene product plastidic with the activity of a “malate dehydrogenase”, preferably being encoded by a gene comprising the nucleic acid sequence SEQ ID NO. 87397, preferably the coding region thereof, conferred the production of or the increase in leucine compared with the wild type control.

The nucleic acid sequence of Slr0721 from Synechocystis sp., e.g. as shown in the respective line in column 5 of Table I, application no. 6, has been published in C. And the activity of the gene product thereof is the activity of malate dehydrogenase.

Accordingly, in one embodiment, the process of the present invention for producing leucine in a non-human organism, like a microorganism or a plant or a part thereof, comprises increasing or generating the activity of a gene product with the activity of a gene product conferring the activity of “malate dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said Slr0721, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr0721, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr0721, or a functional equivalent or a homolog thereof as depicted in column 8 of Table II, application no. 6, preferably a homolog or functional equivalent as depicted in column 8 of Table II B, application no. 6, and being depicted in the same respective line as said Slr0721, and preferably the activity is increased non-targeted, whereby the respective line discloses in column 7 the fine chemical leucine.

Accordingly, in one embodiment, the molecule which activity is to be increased in the process of the invention is the gene product with an activity as a “malate dehydrogenase”, preferably it is the molecule of section (a) or (b) of this paragraph.

In particular, it was observed that in plants, especially in Arabidopsis thaliana, increasing or generating the activity of a gene product non-targeted with the activity of a “malate dehydrogenase”, preferably being encoded by a gene comprising the nucleic acid sequence SEQ ID NO. 87397, preferably the coding region thereof, conferred the production of or the increase in leucine compared with the wild type control.

The nucleic acid sequence of Slr0721 from Synechocystis sp., e.g. as shown in the respective line in column 5 of Table I, application no. 6, has been published in C. And the activity of the gene product thereof is the activity of malate dehydrogenase.

Accordingly, in one embodiment, the process of the present invention for producing valine in a non-human organism, like a microorganism or a plant or a part thereof, comprises increasing or generating the activity of a gene product with the activity of a gene product conferring the activity of “malate dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said Slr0721, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr0721, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr0721, or a functional equivalent or a homolog thereof as depicted in column 8 of Table II, application no. 6, preferably a homolog or functional equivalent as depicted in column 8 of Table II B, application no. 6, and being depicted in the same respective line as said Slr0721, and preferably the activity is increased non-targeted, whereby the respective line discloses in column 7 the fine chemical valine.

Accordingly, in one embodiment, the molecule which activity is to be increased in the process of the invention is the gene product with an activity as a “malate dehydrogenase”, preferably it is the molecule of section (a) or (b) of this paragraph.

In particular, it was observed that in plants, especially in Arabidopsis thaliana, increasing or generating the activity of a gene product non-targeted with the activity of a “malate dehydrogenase”, preferably being encoded by a gene comprising the nucleic acid sequence SEQ ID NO. 87397, preferably the coding region thereof, conferred the production of or the increase in valine compared with the wild type control.

The nucleic acid sequence of Slr0721 from Synechocystis sp., e.g. as shown in the respective line in column 5 of Table I, application no. 6, has been published in C. And the activity of the gene product thereof is the activity of malate dehydrogenase.

Accordingly, in one embodiment, the process of the present invention for producing valine in a non-human organism, like a microorganism or a plant or a part thereof, comprises increasing or generating the activity of a gene product with the activity of a gene product conferring the activity of “malate dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said Slr0721, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr0721, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr0721, or a functional equivalent or a homolog thereof as depicted in column 8 of Table II, application no. 6, preferably a homolog or functional equivalent as depicted in column 8 of Table II B, application no. 6, and being depicted in the same respective line as said Slr0721, and preferably the activity is increased plastidic, whereby the respective line discloses in column 7 the fine chemical valine.

Accordingly, in one embodiment, the molecule which activity is to be increased in the process of the invention is the gene product with an activity as a “malate dehydrogenase”, preferably it is the molecule of section (a) or (b) of this paragraph.

In particular, it was observed that in plants, especially in Arabidopsis thaliana, increasing or generating the activity of a gene product plastidic with the activity of a “malate dehydrogenase”, preferably being encoded by a gene comprising the nucleic acid sequence SEQ ID NO. 87397, preferably the coding region thereof, conferred the production of or the increase in valine compared with the wild type control.

The nucleic acid sequence of Slr0739 from Synechocystis sp., e.g. as shown in the respective line in column 5 of Table I, application no. 6, has been published in C. And the activity of the gene product thereof is the activity of geranylgeranyl pyrophosphate synthase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “geranylgeranyl pyrophosphate synthase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Slr0739, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr0739, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr0739, or a functional equivalent or a homolog thereof as depicted in column 8 of Table II, application no. 6, preferably a homolog or functional equivalent as depicted in column 8 of Table II B, application no. 6, and being depicted in the same respective line as said Slr0739, and preferably the activity is increased plastidic, whereby the respective line discloses in column 7 the fine chemical isoleucine.

Accordingly, in one embodiment, the molecule which activity is to be increased in the process of the invention is the gene product with an activity as a “geranylgeranyl pyrophosphate synthase”, preferably it is the molecule of section (a) or (b) of this paragraph.

In particular, it was observed that in plants, especially in Arabidopsis thaliana, increasing or generating the activity of a gene product plastidic with the activity of a “geranylgeranyl pyrophosphate synthase”, preferably being encoded by a gene comprising the nucleic acid sequence SEQ ID NO. 57235, preferably the coding region thereof, conferred the production of or the increase in isoleucine compared with the wild type control.

The nucleic acid sequence of Slr0739 from Synechocystis sp., e.g. as shown in the respective line in column 5 of Table I, application no. 6, has been published in C. And the activity of the gene product thereof is the activity of geranylgeranyl pyrophosphate synthase.

Accordingly, in one embodiment, the process of the present invention for producing leucine in a non-human organism, like a microorganism or a plant or a part thereof, comprises increasing or generating the activity of a gene product with the activity of a gene product conferring the activity of “geranylgeranyl pyrophosphate synthase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said Slr0739, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr0739, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr0739, or a functional equivalent or a homolog thereof as depicted in column 8 of Table II, application no. 6, preferably a homolog or functional equivalent as depicted in column 8 of Table II B, application no. 6, and being depicted in the same respective line as said Slr0739, and preferably the activity is increased plastidic, whereby the respective line discloses in column 7 the fine chemical leucine.

Accordingly, in one embodiment, the molecule which activity is to be increased in the process of the invention is the gene product with an activity as a “geranylgeranyl pyrophosphate synthase”, preferably it is the molecule of section (a) or (b) of this paragraph.

In particular, it was observed that in plants, especially in Arabidopsis thaliana, increasing or generating the activity of a gene product plastidic with the activity of a “geranylgeranyl pyrophosphate synthase”, preferably being encoded by a gene comprising the nucleic acid sequence SEQ ID NO. 57235, preferably the coding region thereof, conferred the production of or the increase in leucine compared with the wild type control.

The nucleic acid sequence of Slr0739 from Synechocystis sp., e.g. as shown in the respective line in column 5 of Table I, application no. 6, has been published in C. And the activity of the gene product thereof is the activity of geranylgeranyl pyrophosphate synthase.

Accordingly, in one embodiment, the process of the present invention for producing valine in a non-human organism, like a microorganism or a plant or a part thereof, comprises increasing or generating the activity of a gene product with the activity of a gene product conferring the activity of “geranylgeranyl pyrophosphate synthase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said Slr0739, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr0739, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr0739, or a functional equivalent or a homolog thereof as depicted in column 8 of Table II, application no. 6, preferably a homolog or functional equivalent as depicted in column 8 of Table II B, application no. 6, and being depicted in the same respective line as said Slr0739, and preferably the activity is increased plastidic, whereby the respective line discloses in column 7 the fine chemical valine.

Accordingly, in one embodiment, the molecule which activity is to be increased in the process of the invention is the gene product with an activity as a “geranylgeranyl pyrophosphate synthase”, preferably it is the molecule of section (a) or (b) of this paragraph.

In particular, it was observed that in plants, especially in Arabidopsis thaliana, increasing or generating the activity of a gene product plastidic with the activity of a “geranylgeranyl pyrophosphate synthase”, preferably being encoded by a gene comprising the nucleic acid sequence SEQ ID NO. 57235, preferably the coding region thereof, conferred the production of or the increase in valine compared with the wild type control.

The nucleic acid sequence of Slr1492 from Synechocystis sp., e.g. as shown in the respective line in column 5 of Table I, application no. 6, has been published in C. And the activity of the gene product thereof is the activity of iron(III) dicitrate-binding protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “iron(III) dicitrate-binding protein”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Slr1492, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr1492, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr1492, or a functional equivalent or a homolog thereof as depicted in column 8 of Table II, application no. 6, preferably a homolog or functional equivalent as depicted in column 8 of Table II B, application no. 6, and being depicted in the same respective line as said Slr1492, and preferably the activity is increased mitochondrial, whereby the respective line discloses in column 7 the fine chemical isoleucine.

Accordingly, in one embodiment, the molecule which activity is to be increased in the process of the invention is the gene product with an activity as a “iron(III) dicitrate-binding protein”, preferably it is the molecule of section (a) or (b) of this paragraph.

In particular, it was observed that in plants, especially in Arabidopsis thaliana, increasing or generating the activity of a gene product mitochondrial with the activity of a “iron(III) dicitratebinding protein”, preferably being encoded by a gene comprising the nucleic acid sequence SEQ ID NO. 58668, preferably the coding region thereof, conferred the production of or the increase in isoleucine compared with the wild type control.

The nucleic acid sequence of Slr1509 from Synechocystis sp., e.g. as shown in the respective line in column 5 of Table I, application no. 6, has been published in C. And the activity of the gene product thereof is the activity of Na+/K+ transporter.

Accordingly, in one embodiment, the process of the present invention for producing isoleucine in a non-human organism, like a microorganism or a plant or a part thereof, comprises increasing or generating the activity of a gene product with the activity of a gene product conferring the activity of “Na+/K+ transporter”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Slr1509, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr1509, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr1509, or a functional equivalent or a homolog thereof as depicted in column 8 of Table II, application no. 6, preferably a homolog or functional equivalent as depicted in column 8 of Table II B, application no. 6, and being depicted in the same respective line as said Slr1509, and preferably the activity is increased mitochondrial, whereby the respective line discloses in column 7 the fine chemical isoleucine.

Accordingly, in one embodiment, the molecule which activity is to be increased in the process of the invention is the gene product with an activity as a “Na+/K+ transporter”, preferably it is the molecule of section (a) or (b) of this paragraph.

In particular, it was observed that in plants, especially in Arabidopsis thaliana, increasing or generating the activity of a gene product mitochondrial with the activity of a “Na+/K+ transporter”, preferably being encoded by a gene comprising the nucleic acid sequence SEQ ID NO. 88158, preferably the coding region thereof, conferred the production of or the increase in isoleucine compared with the wild type control.

The nucleic acid sequence of Slr1509 from Synechocystis sp., e.g. as shown in the respective line in column 5 of Table I, application no. 6, has been published in C. And the activity of the gene product thereof is the activity of Na+/K+ transporter.

Accordingly, in one embodiment, the process of the present invention for producing leucine in a non-human organism, like a microorganism or a plant or a part thereof, comprises increasing or generating the activity of a gene product with the activity of a gene product conferring the activity of “Na+/K+ transporter”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said Slr1509, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr1509, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr1509, or a functional equivalent or a homolog thereof as depicted in column 8 of Table II, application no. 6, preferably a homolog or functional equivalent as depicted in column 8 of Table II B, application no. 6, and being depicted in the same respective line as said Slr1509, and preferably the activity is increased mitochondrial, whereby the respective line discloses in column 7 the fine chemical leucine.

Accordingly, in one embodiment, the molecule which activity is to be increased in the process of the invention is the gene product with an activity as a “Na+/K+ transporter”, preferably it is the molecule of section (a) or (b) of this paragraph.

In particular, it was observed that in plants, especially in Arabidopsis thaliana, increasing or generating the activity of a gene product mitochondrial with the activity of a “Na+/K+ transporter”, preferably being encoded by a gene comprising the nucleic acid sequence SEQ ID NO. 88158, preferably the coding region thereof, conferred the production of or the increase in leucine compared with the wild type control.

The nucleic acid sequence of Slr1517 from Synechocystis sp., e.g. as shown in the respective line in column 5 of Table I, application no. 6, has been published in C. And the activity of the gene product thereof is the activity of 3-isopropylmalate dehydrogenase.

Accordingly, in one embodiment, the process of the present invention for producing valine in a non-human organism, like a microorganism or a plant or a part thereof, comprises increasing or generating the activity of a gene product with the activity of a gene product conferring the activity of “3-isopropylmalate dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said Slr1517, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr1517, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr1517, or a functional equivalent or a homolog thereof as depicted in column 8 of Table II, application no. 6, preferably a homolog or functional equivalent as depicted in column 8 of Table II B, application no. 6, and being depicted in the same respective line as said Slr1517, and preferably the activity is increased mitochondrial, whereby the respective line discloses in column 7 the fine chemical valine.

Accordingly, in one embodiment, the molecule which activity is to be increased in the process of the invention is the gene product with an activity as a “3-isopropylmalate dehydrogenase”, preferably it is the molecule of section (a) or (b) of this paragraph.

In particular, it was observed that in plants, especially in Arabidopsis thaliana, increasing or generating the activity of a gene product mitochondrial with the activity of a “3-isopropylmalate dehydrogenase”, preferably being encoded by a gene comprising the nucleic acid sequence SEQ ID NO. 107145, preferably the coding region thereof, conferred the production of or the increase in valine compared with the wild type control.

The nucleic acid sequence of Slr1739 from Synechocystis sp., e.g. as shown in the respective line in column 5 of Table I, application no. 6, has been published in C. And the activity of the gene product thereof is the activity of photosystem II protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “photosystem II protein”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Slr1739, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr1739, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr1739, or a functional equivalent or a homolog thereof as depicted in column 8 of Table II, application no. 6, preferably a homolog or functional equivalent as depicted in column 8 of Table II B, application no. 6, and being depicted in the same respective line as said Slr1739, and preferably the activity is increased plastidic, whereby the respective line discloses in column 7 the fine chemical isoleucine.

Accordingly, in one embodiment, the molecule which activity is to be increased in the process of the invention is the gene product with an activity as a “photosystem II protein”, preferably it is the molecule of section (a) or (b) of this paragraph.

In particular, it was observed that in plants, especially in Arabidopsis thaliana, increasing or generating the activity of a gene product plastidic with the activity of a “photosystem II protein”, preferably being encoded by a gene comprising the nucleic acid sequence SEQ ID NO. 58731, preferably the coding region thereof, conferred the production of or the increase in isoleucine compared with the wild type control.

The nucleic acid sequence of Slr1739 from Synechocystis sp., e.g. as shown in the respective line in column 5 of Table I, application no. 6, has been published in C. And the activity of the gene product thereof is the activity of photosystem II protein.

Accordingly, in one embodiment, the process of the present invention for producing leucine in a non-human organism, like a microorganism or a plant or a part thereof, comprises increasing or generating the activity of a gene product with the activity of a gene product conferring the activity of “photosystem II protein”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said Slr1739, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr1739, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr1739, or a functional equivalent or a homolog thereof as depicted in column 8 of Table II, application no. 6, preferably a homolog or functional equivalent as depicted in column 8 of Table II B, application no. 6, and being depicted in the same respective line as said Slr1739, and preferably the activity is increased plastidic, whereby the respective line discloses in column 7 the fine chemical leucine.

Accordingly, in one embodiment, the molecule which activity is to be increased in the process of the invention is the gene product with an activity as a “photosystem II protein”, preferably it is the molecule of section (a) or (b) of this paragraph.

In particular, it was observed that in plants, especially in Arabidopsis thaliana, increasing or generating the activity of a gene product plastidic with the activity of a “photosystem II protein”, preferably being encoded by a gene comprising the nucleic acid sequence SEQ ID NO. 58731, preferably the coding region thereof, conferred the production of or the increase in leucine compared with the wild type control.

The nucleic acid sequence of Slr1739 from Synechocystis sp., e.g. as shown in the respective line in column 5 of Table I, application no. 6, has been published in C. And the activity of the gene product thereof is the activity of photosystem II protein.

Accordingly, in one embodiment, the process of the present invention for producing valine in a non-human organism, like a microorganism or a plant or a part thereof, comprises increasing or generating the activity of a gene product with the activity of a gene product conferring the activity of “photosystem II protein”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said Slr1739, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr1739, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr1739, or a functional equivalent or a homolog thereof as depicted in column 8 of Table II, application no. 6, preferably a homolog or functional equivalent as depicted in column 8 of Table II B, application no. 6, and being depicted in the same respective line as said Slr1739, and preferably the activity is increased plastidic, whereby the respective line discloses in column 7 the fine chemical valine.

Accordingly, in one embodiment, the molecule which activity is to be increased in the process of the invention is the gene product with an activity as a “photosystem II protein”, preferably it is the molecule of section (a) or (b) of this paragraph.

In particular, it was observed that in plants, especially in Arabidopsis thaliana, increasing or generating the activity of a gene product plastidic with the activity of a “photosystem II protein”, preferably being encoded by a gene comprising the nucleic acid sequence SEQ ID NO. 58731, preferably the coding region thereof, conferred the production of or the increase in valine compared with the wild type control.

The nucleic acid sequence of Slr1791 from Synechocystis sp., e.g. as shown in the respective line in column 5 of Table I, application no. 6, has been published in C. And the activity of the gene product thereof is the activity of phosphoadenosine phosphosulfate reductase .

Accordingly, in one embodiment, the process of the present invention for producing valine in a non-human organism, like a microorganism or a plant or a part thereof, comprises increasing or generating the activity of a gene product with the activity of a gene product conferring the activity of “phosphoadenosine phosphosulfate reductase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said Slr1791, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr1791, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr1791, or a functional equivalent or a homolog thereof as depicted in column 8 of Table II, application no. 6, preferably a homolog or functional equivalent as depicted in column 8 of Table II B, application no. 6, and being depicted in the same respective line as said Slr1791, and preferably the activity is increased plastidic, whereby the respective line discloses in column 7 the fine chemical valine.

Accordingly, in one embodiment, the molecule which activity is to be increased in the process of the invention is the gene product with an activity as a “phosphoadenosine phosphosulfate reductase”, preferably it is the molecule of section (a) or (b) of this paragraph.

In particular, it was observed that in plants, especially in Arabidopsis thaliana, increasing or generating the activity of a gene product plastidic with the activity of a “phosphoadenosine phosphosulfate reductase”, preferably being encoded by a gene comprising the nucleic acid sequence SEQ ID NO. 12140, preferably the coding region thereof, conferred the production of or the increase in valine compared with the wild type control.

The nucleic acid sequence of Slr1882 from Synechocystis sp., e.g. as shown in the respective line in column 5 of Table I, application no. 6, has been published in C. And the activity of the gene product thereof is the activity of riboflavin biosynthesis protein.

Accordingly, in one embodiment, the process of the present invention for producing valine in a non-human organism, like a microorganism or a plant or a part thereof, comprises increasing or generating the activity of a gene product with the activity of a gene product conferring the activity of “riboflavin biosynthesis protein”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said Slr1882, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr1882, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr1882, or a functional equivalent or a homolog thereof as depicted in column 8 of Table II, application no. 6, preferably a homolog or functional equivalent as depicted in column 8 of Table II B, application no. 6, and being depicted in the same respective line as said Slr1882, and preferably the activity is increased non-targeted, whereby the respective line discloses in column 7 the fine chemical valine.

Accordingly, in one embodiment, the molecule which activity is to be increased in the process of the invention is the gene product with an activity as a “riboflavin biosynthesis protein”, preferably it is the molecule of section (a) or (b) of this paragraph.

In particular, it was observed that in plants, especially in Arabidopsis thaliana, increasing or generating the activity of a gene product non-targeted with the activity of a “riboflavin biosynthesis protein”, preferably being encoded by a gene comprising the nucleic acid sequence SEQ ID NO. 59041, preferably the coding region thereof, conferred the production of or the increase in valine compared with the wild type control.

The nucleic acid sequence of Slr2072 from Synechocystis sp., e.g. as shown in the respective line in column 5 of Table I, application no. 6, has been published in C. And the activity of the gene product thereof is the activity of threonine dehydratase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “threonine dehydratase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Slr2072, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr2072, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr2072, or a functional equivalent or a homolog thereof as depicted in column 8 of Table II, application no. 6, preferably a homolog or functional equivalent as depicted in column 8 of Table II B, application no. 6, and being depicted in the same respective line as said Slr2072, and preferably the activity is increased mitochondrial, whereby the respective line discloses in column 7 the fine chemical isoleucine.

Accordingly, in one embodiment, the molecule which activity is to be increased in the process of the invention is the gene product with an activity as a “threonine dehydratase”, preferably it is the molecule of section (a) or (b) of this paragraph.

In particular, it was observed that in plants, especially in Arabidopsis thaliana, increasing or generating the activity of a gene product mitochondrial with the activity of a “threonine dehydratase”, preferably being encoded by a gene comprising the nucleic acid sequence SEQ ID NO. 12341, preferably the coding region thereof, conferred the production of or the increase in isoleucine compared with the wild type control.

The nucleic acid sequence of Slr2072 from Synechocystis sp., e.g. as shown in the respective line in column 5 of Table I, application no. 6, has been published in C. And the activity of the gene product thereof is the activity of threonine dehydratase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “threonine dehydratase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Slr2072, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr2072, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr2072, or a functional equivalent or a homolog thereof as depicted in column 8 of Table II, application no. 6, preferably a homolog or functional equivalent as depicted in column 8 of Table II B, application no. 6, and being depicted in the same respective line as said Slr2072, and preferably the activity is increased plastidic, whereby the respective line discloses in column 7 the fine chemical isoleucine.

Accordingly, in one embodiment, the molecule which activity is to be increased in the process of the invention is the gene product with an activity as a “threonine dehydratase”, preferably it is the molecule of section (a) or (b) of this paragraph.

In particular, it was observed that in plants, especially in Arabidopsis thaliana, increasing or generating the activity of a gene product plastidic with the activity of a “threonine dehydratase”, preferably being encoded by a gene comprising the nucleic acid sequence SEQ ID NO. 12341, preferably the coding region thereof, conferred the production of or the increase in isoleucine compared with the wild type control.

The nucleic acid sequence of Slr2072 from Synechocystis sp., e.g. as shown in the respective line in column 5 of Table I, application no. 6, has been published in C. And the activity of the gene product thereof is the activity of threonine dehydratase.

Accordingly, in one embodiment, the process of the present invention for producing leucine in a non-human organism, like a microorganism or a plant or a part thereof, comprises increasing or generating the activity of a gene product with the activity of a gene product conferring the activity of “threonine dehydratase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said Slr2072, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr2072, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr2072, or a functional equivalent or a homolog thereof as depicted in column 8 of Table II, application no. 6, preferably a homolog or functional equivalent as depicted in column 8 of Table II B, application no. 6, and being depicted in the same respective line as said Slr2072, and preferably the activity is increased plastidic, whereby the respective line discloses in column 7 the fine chemical leucine.

Accordingly, in one embodiment, the molecule which activity is to be increased in the process of the invention is the gene product with an activity as a “threonine dehydratase”, preferably it is the molecule of section (a) or (b) of this paragraph.

In particular, it was observed that in plants, especially in Arabidopsis thaliana, increasing or generating the activity of a gene product plastidic with the activity of a “threonine dehydratase”, preferably being encoded by a gene comprising the nucleic acid sequence SEQ ID NO. 12341, preferably the coding region thereof, conferred the production of or the increase in leucine compared with the wild type control.

The nucleic acid sequence of Slr2072 from Synechocystis sp., e.g. as shown in the respective line in column 5 of Table I, application no. 6, has been published in C. And the activity of the gene product thereof is the activity of threonine dehydratase.

Accordingly, in one embodiment, the process of the present invention for producing leucine in a non-human organism, like a microorganism or a plant or a part thereof, comprises increasing or generating the activity of a gene product with the activity of a gene product conferring the activity of “threonine dehydratase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said Slr2072, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr2072, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr2072, or a functional equivalent or a homolog thereof as depicted in column 8 of Table II, application no. 6, preferably a homolog or functional equivalent as depicted in column 8 of Table II B, application no. 6, and being depicted in the same respective line as said Slr2072, and preferably the activity is increased mitochondrial, whereby the respective line discloses in column 7 the fine chemical leucine.

Accordingly, in one embodiment, the molecule which activity is to be increased in the process of the invention is the gene product with an activity as a “threonine dehydratase”, preferably it is the molecule of section (a) or (b) of this paragraph.

In particular, it was observed that in plants, especially in Arabidopsis thaliana, increasing or generating the activity of a gene product mitochondrial with the activity of a “threonine dehydratase”, preferably being encoded by a gene comprising the nucleic acid sequence SEQ ID NO. 12341, preferably the coding region thereof, conferred the production of or the increase in leucine compared with the wild type control.

The nucleic acid sequence of Slr2072 from Synechocystis sp., e.g. as shown in the respective line in column 5 of Table I, application no. 6, has been published in C. And the activity of the gene product thereof is the activity of threonine dehydratase.

Accordingly, in one embodiment, the process of the present invention for producing valine in a non-human organism, like a microorganism or a plant or a part thereof, comprises increasing or generating the activity of a gene product with the activity of a gene product conferring the activity of “threonine dehydratase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said Slr2072, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr2072, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr2072, or a functional equivalent or a homolog thereof as depicted in column 8 of Table II, application no. 6, preferably a homolog or functional equivalent as depicted in column 8 of Table II B, application no. 6, and being depicted in the same respective line as said Slr2072, and preferably the activity is increased mitochondrial, whereby the respective line discloses in column 7 the fine chemical valine.

Accordingly, in one embodiment, the molecule which activity is to be increased in the process of the invention is the gene product with an activity as a “threonine dehydratase”, preferably it is the molecule of section (a) or (b) of this paragraph.

In particular, it was observed that in plants, especially in Arabidopsis thaliana, increasing or generating the activity of a gene product mitochondrial with the activity of a “threonine dehydratase”, preferably being encoded by a gene comprising the nucleic acid sequence SEQ ID NO. 12341, preferably the coding region thereof, conferred the production of or the increase in valine compared with the wild type control.

The nucleic acid sequence of Slr2072 from Synechocystis sp., e.g. as shown in the respective line in column 5 of Table I, application no. 6, has been published in C. And the activity of the gene product thereof is the activity of threonine dehydratase.

Accordingly, in one embodiment, the process of the present invention for producing valine in a non-human organism, like a microorganism or a plant or a part thereof, comprises increasing or generating the activity of a gene product with the activity of a gene product conferring the activity of “threonine dehydratase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said Slr2072, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr2072, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr2072, or a functional equivalent or a homolog thereof as depicted in column 8 of Table II, application no. 6, preferably a homolog or functional equivalent as depicted in column 8 of Table II B, application no. 6, and being depicted in the same respective line as said Slr2072, and preferably the activity is increased plastidic, whereby the respective line discloses in column 7 the fine chemical valine.

Accordingly, in one embodiment, the molecule which activity is to be increased in the process of the invention is the gene product with an activity as a “threonine dehydratase”, preferably it is the molecule of section (a) or (b) of this paragraph.

In particular, it was observed that in plants, especially in Arabidopsis thaliana, increasing or generating the activity of a gene product plastidic with the activity of a “threonine dehydratase”, preferably being encoded by a gene comprising the nucleic acid sequence SEQ ID NO. 12341, preferably the coding region thereof, conferred the production of or the increase in valine compared with the wild type control.

The nucleic acid sequence of Slr2124 from Synechocystis sp., e.g. as shown in the respective line in column 5 of Table I, application no. 6, has been published in C. And the activity of the gene product thereof is the activity of short-chain alcohol dehydrogenase family.

Accordingly, in one embodiment, the process of the present invention for producing valine in a non-human organism, like a microorganism or a plant or a part thereof, comprises increasing or generating the activity of a gene product with the activity of a gene product conferring the activity of “short-chain alcohol dehydrogenase family”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said Slr2124, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Slr2124, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Slr2124, or a functional equivalent or a homolog thereof as depicted in column 8 of Table II, application no. 6, preferably a homolog or functional equivalent as depicted in column 8 of Table II B, application no. 6, and being depicted in the same respective line as said Slr2124, and preferably the activity is increased plastidic, whereby the respective line discloses in column 7 the fine chemical valine.

Accordingly, in one embodiment, the molecule which activity is to be increased in the process of the invention is the gene product with an activity as a “short-chain alcohol dehydrogenase family”, preferably it is the molecule of section (a) or (b) of this paragraph.

In particular, it was observed that in plants, especially in Arabidopsis thaliana, increasing or generating the activity of a gene product plastidic with the activity of a “short-chain alcohol dehydrogenase family”, preferably being encoded by a gene comprising the nucleic acid sequence SEQ ID NO. 59370, preferably the coding region thereof, conferred the production of or the increase in valine compared with the wild type control.

The nucleic acid sequence of TTC0019 from Thermus thermophilus, e.g. as shown in the respective line in column 5 of Table I, application no. 6, has been published in E. And the activity of the gene product thereof is the activity of Sec-independent protein translocase subunit.

Accordingly, in one embodiment, the process of the present invention for producing isoleucine in a non-human organism, like a microorganism or a plant or a part thereof, comprises increasing or generating the activity of a gene product with the activity of a gene product conferring the activity of “Sec-independent protein translocase subunit”, especially from Thermus thermophilus or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said TTC0019, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said TTC0019, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said TTC0019, or a functional equivalent or a homolog thereof as depicted in column 8 of Table II, application no. 6, preferably a homolog or functional equivalent as depicted in column 8 of Table II B, application no. 6, and being depicted in the same respective line as said TTC0019, and preferably the activity is increased non-targeted, whereby the respective line discloses in column 7 the fine chemical isoleucine.

Accordingly, in one embodiment, the molecule which activity is to be increased in the process of the invention is the gene product with an activity as a “Sec-independent protein translocase subunit”, preferably it is the molecule of section (a) or (b) of this paragraph.

In particular, it was observed that in plants, especially in Arabidopsis thaliana, increasing or generating the activity of a gene product non-targeted with the activity of a “Sec-independent protein translocase subunit”, preferably being encoded by a gene comprising the nucleic acid sequence SEQ ID NO. 12698, preferably the coding region thereof, conferred the production of or the increase in isoleucine compared with the wild type control.

The nucleic acid sequence of TTC0019 from Thermus thermophilus, e.g. as shown in the respective line in column 5 of Table I, application no. 6, has been published in E. And the activity of the gene product thereof is the activity of Sec-independent protein translocase subunit.

Accordingly, in one embodiment, the process of the present invention for producing leucine in a non-human organism, like a microorganism or a plant or a part thereof, comprises increasing or generating the activity of a gene product with the activity of a gene product conferring the activity of “Sec-independent protein translocase subunit”, especially from Thermus thermophilus or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said TTC0019, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said TTC0019, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said TTC0019, or a functional equivalent or a homolog thereof as depicted in column 8 of Table II, application no. 6, preferably a homolog or functional equivalent as depicted in column 8 of Table II B, application no. 6, and being depicted in the same respective line as said TTC0019, and preferably the activity is increased non-targeted, whereby the respective line discloses in column 7 the fine chemical leucine.

Accordingly, in one embodiment, the molecule which activity is to be increased in the process of the invention is the gene product with an activity as a “Sec-independent protein translocase subunit”, preferably it is the molecule of section (a) or (b) of this paragraph.

In particular, it was observed that in plants, especially in Arabidopsis thaliana, increasing or generating the activity of a gene product non-targeted with the activity of a “Sec-independent protein translocase subunit”, preferably being encoded by a gene comprising the nucleic acid sequence SEQ ID NO. 12698, preferably the coding region thereof, conferred the production of or the increase in leucine compared with the wild type control.

The nucleic acid sequence of TTC0019 from Thermus thermophilus, e.g. as shown in the respective line in column 5 of Table I, application no. 6, has been published in E. And the activity of the gene product thereof is the activity of Sec-independent protein translocase subunit.

Accordingly, in one embodiment, the process of the present invention for producing valine in a non-human organism, like a microorganism or a plant or a part thereof, comprises increasing or generating the activity of a gene product with the activity of a gene product conferring the activity of “Sec-independent protein translocase subunit”, especially from Thermus thermophilus or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said TTC0019, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said TTC0019, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said TTC0019, or a functional equivalent or a homolog thereof as depicted in column 8 of Table II, application no. 6, preferably a homolog or functional equivalent as depicted in column 8 of Table II B, application no. 6, and being depicted in the same respective line as said TTC0019, and preferably the activity is increased non-targeted, whereby the respective line discloses in column 7 the fine chemical valine.

Accordingly, in one embodiment, the molecule which activity is to be increased in the process of the invention is the gene product with an activity as a “Sec-independent protein translocase subunit”, preferably it is the molecule of section (a) or (b) of this paragraph.

In particular, it was observed that in plants, especially in Arabidopsis thaliana, increasing or generating the activity of a gene product non-targeted with the activity of a “Sec-independent protein translocase subunit”, preferably being encoded by a gene comprising the nucleic acid sequence SEQ ID NO. 12698, preferably the coding region thereof, conferred the production of or the increase in valine compared with the wild type control.

The nucleic acid sequence of TTC0849 from Thermus thermophilus, e.g. as shown in the respective line in column 5 of Table I, application no. 6, has been published in E. And the activity of the gene product thereof is the activity of 2-isopropylmalate synthase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “2-isopropylmalate synthase”, especially from Thermus thermophilus or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said TTC0849, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said TTC0849, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said TTC0849, or a functional equivalent or a homolog thereof as depicted in column 8 of Table II, application no. 6, preferably a homolog or functional equivalent as depicted in column 8 of Table II B, application no. 6, and being depicted in the same respective line as said TTC0849, and preferably the activity is increased non-targeted, whereby the respective line discloses in column 7 the fine chemical isoleucine.

Accordingly, in one embodiment, the molecule which activity is to be increased in the process of the invention is the gene product with an activity as a “2-isopropylmalate synthase”, preferably it is the molecule of section (a) or (b) of this paragraph.

In particular, it was observed that in plants, especially in Arabidopsis thaliana, increasing or generating the activity of a gene product non-targeted with the activity of a “2-isopropylmalate synthase”, preferably being encoded by a gene comprising the nucleic acid sequence SEQ ID NO. 107556, preferably the coding region thereof, conferred the production of or the increase in isoleucine compared with the wild type control.

The nucleic acid sequence of TTC0849 from Thermus thermophilus, e.g. as shown in the respective line in column 5 of Table I, application no. 6, has been published in E. And the activity of the gene product thereof is the activity of 2-isopropylmalate synthase.

Accordingly, in one embodiment, the process of the present invention for producing leucine in a non-human organism, like a microorganism or a plant or a part thereof, comprises increasing or generating the activity of a gene product with the activity of a gene product conferring the activity of “2-isopropylmalate synthase”, especially from Thermus thermophilus or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said TTC0849, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said TTC0849, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said TTC0849, or a functional equivalent or a homolog thereof as depicted in column 8 of Table II, application no. 6, preferably a homolog or functional equivalent as depicted in column 8 of Table II B, application no. 6, and being depicted in the same respective line as said TTC0849, and preferably the activity is increased non-targeted, whereby the respective line discloses in column 7 the fine chemical leucine.

Accordingly, in one embodiment, the molecule which activity is to be increased in the process of the invention is the gene product with an activity as a “2-isopropylmalate synthase”, preferably it is the molecule of section (a) or (b) of this paragraph.

In particular, it was observed that in plants, especially in Arabidopsis thaliana, increasing or generating the activity of a gene product non-targeted with the activity of a “2-isopropylmalate synthase”, preferably being encoded by a gene comprising the nucleic acid sequence SEQ ID NO. 107556, preferably the coding region thereof, conferred the production of or the increase in leucine compared with the wild type control.

The nucleic acid sequence of TTC1386 from Thermus thermophilus, e.g. as shown in the respective line in column 5 of Table I, application no. 6, has been published in E. And the activity of the gene product thereof is the activity of TTC1386-protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “TTC1386-protein”, especially from Thermus thermophilus or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said TTC1386, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said TTC1386, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said TTC1386, or a functional equivalent or a homolog thereof as depicted in column 8 of Table II, application no. 6, preferably a homolog or functional equivalent as depicted in column 8 of Table II B, application no. 6, and being depicted in the same respective line as said TTC1386, and preferably the activity is increased non-targeted, whereby the respective line discloses in column 7 the fine chemical isoleucine.

Accordingly, in one embodiment, the molecule which activity is to be increased in the process of the invention is the gene product with an activity as a “TTC1386-protein”, preferably it is the molecule of section (a) or (b) of this paragraph.

In particular, it was observed that in plants, especially in Arabidopsis thaliana, increasing or generating the activity of a gene product non-targeted with the activity of a “TTC1386-protein”, preferably being encoded by a gene comprising the nucleic acid sequence SEQ ID NO. 62079, preferably the coding region thereof, conferred the production of or the increase in isoleucine compared with the wild type control.

The nucleic acid sequence of TTC1386 from Thermus thermophilus, e.g. as shown in the respective line in column 5 of Table I, application no. 6, has been published in E. And the activity of the gene product thereof is the activity of TTC1386-protein.

Accordingly, in one embodiment, the process of the present invention for producing leucine in a non-human organism, like a microorganism or a plant or a part thereof, comprises increasing or generating the activity of a gene product with the activity of a gene product conferring the activity of “TTC1386-protein”, especially from Thermus thermophilus or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said TTC1386, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said TTC1386, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said TTC1386, or a functional equivalent or a homolog thereof as depicted in column 8 of Table II, application no. 6, preferably a homolog or functional equivalent as depicted in column 8 of Table II B, application no. 6, and being depicted in the same respective line as said TTC1386, and preferably the activity is increased non-targeted, whereby the respective line discloses in column 7 the fine chemical leucine.

Accordingly, in one embodiment, the molecule which activity is to be increased in the process of the invention is the gene product with an activity as a “TTC1386-protein”, preferably it is the molecule of section (a) or (b) of this paragraph.

In particular, it was observed that in plants, especially in Arabidopsis thaliana, increasing or generating the activity of a gene product non-targeted with the activity of a “TTC1386-protein”, preferably being encoded by a gene comprising the nucleic acid sequence SEQ ID NO. 62079, preferably the coding region thereof, conferred the production of or the increase in leucine compared with the wild type control.

The nucleic acid sequence of TTC1386 from Thermus thermophilus, e.g. as shown in the respective line in column 5 of Table I, application no. 6, has been published in E. And the activity of the gene product thereof is the activity of TTC1386-protein.

Accordingly, in one embodiment, the process of the present invention for producing valine in a non-human organism, like a microorganism or a plant or a part thereof, comprises increasing or generating the activity of a gene product with the activity of a gene product conferring the activity of “TTC1386-protein”, especially from Thermus thermophilus or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said TTC1386, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said TTC1386, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said TTC1386, or a functional equivalent or a homolog thereof as depicted in column 8 of Table II, application no. 6, preferably a homolog or functional equivalent as depicted in column 8 of Table II B, application no. 6, and being depicted in the same respective line as said TTC1386, and preferably the activity is increased non-targeted, whereby the respective line discloses in column 7 the fine chemical valine.

Accordingly, in one embodiment, the molecule which activity is to be increased in the process of the invention is the gene product with an activity as a “TTC1386-protein”, preferably it is the molecule of section (a) or (b) of this paragraph.

In particular, it was observed that in plants, especially in Arabidopsis thaliana, increasing or generating the activity of a gene product non-targeted with the activity of a “TTC1386-protein”, preferably being encoded by a gene comprising the nucleic acid sequence SEQ ID NO. 62079, preferably the coding region thereof, conferred the production of or the increase in valine compared with the wild type control.

The nucleic acid sequence of TTC1550 from Thermus thermophilus, e.g. as shown in the respective line in column 5 of Table I, application no. 6, has been published in E. And the activity of the gene product thereof is the activity of homocitrate synthase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “homocitrate synthase”, especially from Thermus thermophilus or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said TTC1550, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said TTC1550, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said TTC1550, or a functional equivalent or a homolog thereof as depicted in column 8 of Table II, application no. 6, preferably a homolog or functional equivalent as depicted in column 8 of Table II B, application no. 6, and being depicted in the same respective line as said TTC1550, and preferably the activity is increased non-targeted, whereby the respective line discloses in column 7 the fine chemical isoleucine.

Accordingly, in one embodiment, the molecule which activity is to be increased in the process of the invention is the gene product with an activity as a “homocitrate synthase”, preferably it is the molecule of section (a) or (b) of this paragraph.

In particular, it was observed that in plants, especially in Arabidopsis thaliana, increasing or generating the activity of a gene product non-targeted with the activity of a “homocitrate synthase”, preferably being encoded by a gene comprising the nucleic acid sequence SEQ ID NO. 12974, preferably the coding region thereof, conferred the production of or the increase in isoleucine compared with the wild type control.

The nucleic acid sequence of TTC1550 from Thermus thermophilus, e.g. as shown in the respective line in column 5 of Table I, application no. 6, has been published in E. And the activity of the gene product thereof is the activity of homocitrate synthase.

Accordingly, in one embodiment, the process of the present invention for producing leucine in a non-human organism, like a microorganism or a plant or a part thereof, comprises increasing or generating the activity of a gene product with the activity of a gene product conferring the activity of “homocitrate synthase”, especially from Thermus thermophilus or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said TTC1550, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said TTC1550, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said TTC1550, or a functional equivalent or a homolog thereof as depicted in column 8 of Table II, application no. 6, preferably a homolog or functional equivalent as depicted in column 8 of Table II B, application no. 6, and being depicted in the same respective line as said TTC1550, and preferably the activity is increased non-targeted, whereby the respective line discloses in column 7 the fine chemical leucine.

Accordingly, in one embodiment, the molecule which activity is to be increased in the process of the invention is the gene product with an activity as a “homocitrate synthase”, preferably it is the molecule of section (a) or (b) of this paragraph.

In particular, it was observed that in plants, especially in Arabidopsis thaliana, increasing or generating the activity of a gene product non-targeted with the activity of a “homocitrate synthase”, preferably being encoded by a gene comprising the nucleic acid sequence SEQ ID NO. 12974, preferably the coding region thereof, conferred the production of or the increase in leucine compared with the wild type control.

The nucleic acid sequence of TTC1550 from Thermus thermophilus, e.g. as shown in the respective line in column 5 of Table I, application no. 6, has been published in E. And the activity of the gene product thereof is the activity of homocitrate synthase.

Accordingly, in one embodiment, the process of the present invention for producing valine in a non-human organism, like a microorganism or a plant or a part thereof, comprises increasing or generating the activity of a gene product with the activity of a gene product conferring the activity of “homocitrate synthase”, especially from Thermus thermophilus or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said TTC1550, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said TTC1550, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said TTC1550, or a functional equivalent or a homolog thereof as depicted in column 8 of Table II, application no. 6, preferably a homolog or functional equivalent as depicted in column 8 of Table II B, application no. 6, and being depicted in the same respective line as said TTC1550, and preferably the activity is increased non-targeted, whereby the respective line discloses in column 7 the fine chemical valine.

Accordingly, in one embodiment, the molecule which activity is to be increased in the process of the invention is the gene product with an activity as a “homocitrate synthase”, preferably it is the molecule of section (a) or (b) of this paragraph.

In particular, it was observed that in plants, especially in Arabidopsis thaliana, increasing or generating the activity of a gene product non-targeted with the activity of a “homocitrate synthase”, preferably being encoded by a gene comprising the nucleic acid sequence SEQ ID NO. 12974, preferably the coding region thereof, conferred the production of or the increase in valine compared with the wild type control.

The nucleic acid sequence of Yal013w from Saccharomyces cerevisiae, e.g. as shown in the respective line in column 5 of Table I, application no. 6, has been published in A. And the activity of the gene product thereof is the activity of YAL013W-protein.

Accordingly, in one embodiment, the process of the present invention for producing leucine in a non-human organism, like a microorganism or a plant or a part thereof, comprises increasing or generating the activity of a gene product with the activity of a gene product conferring the activity of “YAL013W-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said Yal013w, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Yal013w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Yal013w, or a functional equivalent or a homolog thereof as depicted in column 8 of Table II, application no. 6, preferably a homolog or functional equivalent as depicted in column 8 of Table II B, application no. 6, and being depicted in the same respective line as said Yal013w, and preferably the activity is increased non-targeted, whereby the respective line discloses in column 7 the fine chemical leucine.

Accordingly, in one embodiment, the molecule which activity is to be increased in the process of the invention is the gene product with an activity as a “YAL013W-protein”, preferably it is the molecule of section (a) or (b) of this paragraph.

In particular, it was observed that in plants, especially in Arabidopsis thaliana, increasing or generating the activity of a gene product non-targeted with the activity of a “YAL013W-protein”, preferably being encoded by a gene comprising the nucleic acid sequence SEQ ID NO. 108092, preferably the coding region thereof, conferred the production of or the increase in leucine compared with the wild type control.

The nucleic acid sequence of Ybr160w from Saccharomyces cerevisiae, e.g. as shown in the respective line in column 5 of Table I, application no. 6, has been published in A. And the activity of the gene product thereof is the activity of cell division control protein.

Accordingly, in one embodiment, the process of the present invention for producing valine in a non-human organism, like a microorganism or a plant or a part thereof, comprises increasing or generating the activity of a gene product with the activity of a gene product conferring the activity of “cell division control protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said Ybr160w, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Ybr160w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Ybr160w, or a functional equivalent or a homolog thereof as depicted in column 8 of Table II, application no. 6, preferably a homolog or functional equivalent as depicted in column 8 of Table II B, application no. 6, and being depicted in the same respective line as said Ybr160w, and preferably the activity is increased non-targeted, whereby the respective line discloses in column 7 the fine chemical valine.

Accordingly, in one embodiment, the molecule which activity is to be increased in the process of the invention is the gene product with an activity as a “cell division control protein”, preferably it is the molecule of section (a) or (b) of this paragraph.

In particular, it was observed that in plants, especially in Arabidopsis thaliana, increasing or generating the activity of a gene product non-targeted with the activity of a “cell division control protein”, preferably being encoded by a gene comprising the nucleic acid sequence SEQ ID NO. 62717, preferably the coding region thereof, conferred the production of or the increase in valine compared with the wild type control.

The nucleic acid sequence of Ycr012w from Saccharomyces cerevisiae, e.g. as shown in the respective line in column 5 of Table I, application no. 6, has been published in A. And the activity of the gene product thereof is the activity of phosphoglycerate kinase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “phosphoglycerate kinase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Ycr012w, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Ycr012w, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Ycr012w, or a functional equivalent or a homolog thereof as depicted in column 8 of Table II, application no. 6, preferably a homolog or functional equivalent as depicted in column 8 of Table II B, application no. 6, and being depicted in the same respective line as said Ycr012w, and preferably the activity is increased plastidic, whereby the respective line discloses in column 7 the fine chemical isoleucine.

Accordingly, in one embodiment, the molecule which activity is to be increased in the process of the invention is the gene product with an activity as a “phosphoglycerate kinase”, preferably it is the molecule of section (a) or (b) of this paragraph.

In particular, it was observed that in plants, especially in Arabidopsis thaliana, increasing or generating the activity of a gene product plastidic with the activity of a “phosphoglycerate kinase”, preferably being encoded by a gene comprising the nucleic acid sequence SEQ ID NO. 108113, preferably the coding region thereof, conferred the production of or the increase in isoleucine compared with the wild type control.

The nucleic acid sequence of Ycr012w from Saccharomyces cerevisiae, e.g. as shown in the respective line in column 5 of Table I, application no. 6, has been published in A. And the activity of the gene product thereof is the activity of phosphoglycerate kinase.

Accordingly, in one embodiment, the process of the present invention for producing leucine in a non-human organism, like a microorganism or a plant or a part thereof, comprises increasing or generating the activity of a gene product with the activity of a gene product conferring the activity of “phosphoglycerate kinase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said Ycr012w, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Ycr012w, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Ycr012w, or a functional equivalent or a homolog thereof as depicted in column 8 of Table II, application no. 6, preferably a homolog or functional equivalent as depicted in column 8 of Table II B, application no. 6, and being depicted in the same respective line as said Ycr012w, and preferably the activity is increased plastidic, whereby the respective line discloses in column 7 the fine chemical leucine.

Accordingly, in one embodiment, the molecule which activity is to be increased in the process of the invention is the gene product with an activity as a “phosphoglycerate kinase”, preferably it is the molecule of section (a) or (b) of this paragraph.

In particular, it was observed that in plants, especially in Arabidopsis thaliana, increasing or generating the activity of a gene product plastidic with the activity of a “phosphoglycerate kinase”, preferably being encoded by a gene comprising the nucleic acid sequence SEQ ID NO. 108113, preferably the coding region thereof, conferred the production of or the increase in leucine compared with the wild type control.

The nucleic acid sequence of Ydr046c from Saccharomyces cerevisiae, e.g. as shown in the respective line in column 5 of Table I, application no. 6, has been published in A. And the activity of the gene product thereof is the activity of branched-chain amino acid permease.

Accordingly, in one embodiment, the process of the present invention for producing isoleucine in a non-human organism, like a microorganism or a plant or a part thereof, comprises increasing or generating the activity of a gene product with the activity of a gene product conferring the activity of “branched-chain amino acid permease”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Ydr046c, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Ydr046c, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Ydr046c, or a functional equivalent or a homolog thereof as depicted in column 8 of Table II, application no. 6, preferably a homolog or functional equivalent as depicted in column 8 of Table II B, application no. 6, and being depicted in the same respective line as said Ydr046c, and preferably the activity is increased plastidic, whereby the respective line discloses in column 7 the fine chemical isoleucine.

Accordingly, in one embodiment, the molecule which activity is to be increased in the process of the invention is the gene product with an activity as a “branched-chain amino acid permease”, preferably it is the molecule of section (a) or (b) of this paragraph.

In particular, it was observed that in plants, especially in Arabidopsis thaliana, increasing or generating the activity of a gene product plastidic with the activity of a “branched-chain amino acid permease”, preferably being encoded by a gene comprising the nucleic acid sequence SEQ ID NO. 63544, preferably the coding region thereof, conferred the production of or the increase in isoleucine compared with the wild type control.

The nucleic acid sequence of Ydr046c from Saccharomyces cerevisiae, e.g. as shown in the respective line in column 5 of Table I, application no. 6, has been published in A. And the activity of the gene product thereof is the activity of branched-chain amino acid permease.

Accordingly, in one embodiment, the process of the present invention for producing valine in a non-human organism, like a microorganism or a plant or a part thereof, comprises increasing or generating the activity of a gene product with the activity of a gene product conferring the activity of “branched-chain amino acid permease”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said Ydr046c, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Ydr046c, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Ydr046c, or a functional equivalent or a homolog thereof as depicted in column 8 of Table II, application no. 6, preferably a homolog or functional equivalent as depicted in column 8 of Table II B, application no. 6, and being depicted in the same respective line as said Ydr046c, and preferably the activity is increased plastidic, whereby the respective line discloses in column 7 the fine chemical valine.

Accordingly, in one embodiment, the molecule which activity is to be increased in the process of the invention is the gene product with an activity as a “branched-chain amino acid permease”, preferably it is the molecule of section (a) or (b) of this paragraph.

In particular, it was observed that in plants, especially in Arabidopsis thaliana, increasing or generating the activity of a gene product plastidic with the activity of a “branched-chain amino acid permease”, preferably being encoded by a gene comprising the nucleic acid sequence SEQ ID NO. 63544, preferably the coding region thereof, conferred the production of or the increase in valine compared with the wild type control.

The nucleic acid sequence of Ydr183w from Saccharomyces cerevisiae, e.g. as shown in the respective line in column 5 of Table I, application no. 6, has been published in A. And the activity of the gene product thereof is the activity of ydr183w-protein.

Accordingly, in one embodiment, the process of the present invention for producing valine in a non-human organism, like a microorganism or a plant or a part thereof, comprises increasing or generating the activity of a gene product with the activity of a gene product conferring the activity of “ydr183w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said Ydr183w, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Ydr183w, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Ydr183w, or a functional equivalent or a homolog thereof as depicted in column 8 of Table II, application no. 6, preferably a homolog or functional equivalent as depicted in column 8 of Table II B, application no. 6, and being depicted in the same respective line as said Ydr183w, and preferably the activity is increased plastidic, whereby the respective line discloses in column 7 the fine chemical valine.

Accordingly, in one embodiment, the molecule which activity is to be increased in the process of the invention is the gene product with an activity as a “ydr183w-protein”, preferably it is the molecule of section (a) or (b) of this paragraph.

In particular, it was observed that in plants, especially in Arabidopsis thaliana, increasing or generating the activity of a gene product plastidic with the activity of a “ydr183w-protein”, preferably being encoded by a gene comprising the nucleic acid sequence SEQ ID NO. 90103, preferably the coding region thereof, conferred the production of or the increase in valine compared with the wild type control.

The nucleic acid sequence of Yel069c from Saccharomyces cerevisiae, e.g. as shown in the respective line in column 5 of Table I, application no. 6, has been published in A. And the activity of the gene product thereof is the activity of hexose transporter.

Accordingly, in one embodiment, the process of the present invention for producing isoleucine in a non-human organism, like a microorganism or a plant or a part thereof, comprises increasing or generating the activity of a gene product with the activity of a gene product conferring the activity of “hexose transporter”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Yel069c, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Yel069c, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Yel069c, or a functional equivalent or a homolog thereof as depicted in column 8 of Table II, application no. 6, preferably a homolog or functional equivalent as depicted in column 8 of Table II B, application no. 6, and being depicted in the same respective line as said Yel069c, and preferably the activity is increased plastidic, whereby the respective line discloses in column 7 the fine chemical isoleucine.

Accordingly, in one embodiment, the molecule which activity is to be increased in the process of the invention is the gene product with an activity as a “hexose transporter”, preferably it is the molecule of section (a) or (b) of this paragraph.

In particular, it was observed that in plants, especially in Arabidopsis thaliana, increasing or generating the activity of a gene product plastidic with the activity of a “hexose transporter”, preferably being encoded by a gene comprising the nucleic acid sequence SEQ ID NO. 97298, preferably the coding region thereof, conferred the production of or the increase in isoleucine compared with the wild type control.

The nucleic acid sequence of Ygl237c from Saccharomyces cerevisiae, e.g. as shown in the respective line in column 5 of Table I, application no. 6, has been published in A. And the activity of the gene product thereof is the activity of ygl237c-protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “ygl237c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Ygl237c, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Ygl237c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Ygl237c, or a functional equivalent or a homolog thereof as depicted in column 8 of Table II, application no. 6, preferably a homolog or functional equivalent as depicted in column 8 of Table II B, application no. 6, and being depicted in the same respective line as said Ygl237c, and preferably the activity is increased non-targeted, whereby the respective line discloses in column 7 the fine chemical isoleucine.

Accordingly, in one embodiment, the molecule which activity is to be increased in the process of the invention is the gene product with an activity as a “ygl237c-protein”, preferably it is the molecule of section (a) or (b) of this paragraph.

In particular, it was observed that in plants, especially in Arabidopsis thaliana, increasing or generating the activity of a gene product non-targeted with the activity of a “ygl237c-protein”, preferably being encoded by a gene comprising the nucleic acid sequence SEQ ID NO. 64177, preferably the coding region thereof, conferred the production of or the increase in isoleucine compared with the wild type control.

The nucleic acid sequence of Ygl237c from Saccharomyces cerevisiae, e.g. as shown in the respective line in column 5 of Table I, application no. 6, has been published in A. And the activity of the gene product thereof is the activity of ygl237c-protein.

Accordingly, in one embodiment, the process of the present invention for producing leucine in a non-human organism, like a microorganism or a plant or a part thereof, comprises increasing or generating the activity of a gene product with the activity of a gene product conferring the activity of “ygl237c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said Ygl237c, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Ygl237c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Ygl237c, or a functional equivalent or a homolog thereof as depicted in column 8 of Table II, application no. 6, preferably a homolog or functional equivalent as depicted in column 8 of Table II B, application no. 6, and being depicted in the same respective line as said Ygl237c, and preferably the activity is increased non-targeted, whereby the respective line discloses in column 7 the fine chemical leucine.

Accordingly, in one embodiment, the molecule which activity is to be increased in the process of the invention is the gene product with an activity as a “ygl237c-protein”, preferably it is the molecule of section (a) or (b) of this paragraph.

In particular, it was observed that in plants, especially in Arabidopsis thaliana, increasing or generating the activity of a gene product non-targeted with the activity of a “ygl237c-protein”, preferably being encoded by a gene comprising the nucleic acid sequence SEQ ID NO. 64177, preferably the coding region thereof, conferred the production of or the increase in leucine compared with the wild type control.

The nucleic acid sequence of Ygr193c from Saccharomyces cerevisiae, e.g. as shown in the respective line in column 5 of Table I, application no. 6, has been published in A. And the activity of the gene product thereof is the activity of YGR193C-protein.

Accordingly, in one embodiment, the process of the present invention for producing leucine in a non-human organism, like a microorganism or a plant or a part thereof, comprises increasing or generating the activity of a gene product with the activity of a gene product conferring the activity of “YGR193C-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said Ygr193c, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Ygr193c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Ygr193c, or a functional equivalent or a homolog thereof as depicted in column 8 of Table II, application no. 6, preferably a homolog or functional equivalent as depicted in column 8 of Table II B, application no. 6, and being depicted in the same respective line as said Ygr193c, and preferably the activity is increased non-targeted, whereby the respective line discloses in column 7 the fine chemical leucine.

Accordingly, in one embodiment, the molecule which activity is to be increased in the process of the invention is the gene product with an activity as a “YGR193C-protein”, preferably it is the molecule of section (a) or (b) of this paragraph.

In particular, it was observed that in plants, especially in Arabidopsis thaliana, increasing or generating the activity of a gene product non-targeted with the activity of a “YGR193C-protein”, preferably being encoded by a gene comprising the nucleic acid sequence SEQ ID NO. 108487, preferably the coding region thereof, conferred the production of or the increase in leucine compared with the wild type control.

The nucleic acid sequence of Yhr006w from Saccharomyces cerevisiae, e.g. as shown in the respective line in column 5 of Table I, application no. 6, has been published in A. And the activity of the gene product thereof is the activity of yhr006w-protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “yhr006w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Yhr006w, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Yhr006w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Yhr006w, or a functional equivalent or a homolog thereof as depicted in column 8 of Table II, application no. 6, preferably a homolog or functional equivalent as depicted in column 8 of Table II B, application no. 6, and being depicted in the same respective line as said Yhr006w, and preferably the activity is increased non-targeted, whereby the respective line discloses in column 7 the fine chemical isoleucine.

Accordingly, in one embodiment, the molecule which activity is to be increased in the process of the invention is the gene product with an activity as a “yhr006w-protein”, preferably it is the molecule of section (a) or (b) of this paragraph.

In particular, it was observed that in plants, especially in Arabidopsis thaliana, increasing or generating the activity of a gene product non-targeted with the activity of a “yhr006w-protein”, preferably being encoded by a gene comprising the nucleic acid sequence SEQ ID NO. 90284, preferably the coding region thereof, conferred the production of or the increase in isoleucine compared with the wild type control.

The nucleic acid sequence of Yhr006w from Saccharomyces cerevisiae, e.g. as shown in the respective line in column 5 of Table I, application no. 6, has been published in A. And the activity of the gene product thereof is the activity of yhr006w-protein.

Accordingly, in one embodiment, the process of the present invention for producing leucine in a non-human organism, like a microorganism or a plant or a part thereof, comprises increasing or generating the activity of a gene product with the activity of a gene product conferring the activity of “yhr006w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said Yhr006w, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Yhr006w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Yhr006w, or a functional equivalent or a homolog thereof as depicted in column 8 of Table II, application no. 6, preferably a homolog or functional equivalent as depicted in column 8 of Table II B, application no. 6, and being depicted in the same respective line as said Yhr006w, and preferably the activity is increased non-targeted, whereby the respective line discloses in column 7 the fine chemical leucine.

Accordingly, in one embodiment, the molecule which activity is to be increased in the process of the invention is the gene product with an activity as a “yhr006w-protein”, preferably it is the molecule of section (a) or (b) of this paragraph.

In particular, it was observed that in plants, especially in Arabidopsis thaliana, increasing or generating the activity of a gene product non-targeted with the activity of a “yhr006w-protein”, preferably being encoded by a gene comprising the nucleic acid sequence SEQ ID NO. 90284, preferably the coding region thereof, conferred the production of or the increase in leucine compared with the wild type control.

The nucleic acid sequence of Yhr072w-a from Saccharomyces cerevisiae, e.g. as shown in the respective line in column 5 of Table I, application no. 6, has been published in A. And the activity of the gene product thereof is the activity of H/ACA ribonucleoprotein complex subunit 3.

Accordingly, in one embodiment, the process of the present invention for producing isoleucine in a non-human organism, like a microorganism or a plant or a part thereof, comprises increasing or generating the activity of a gene product with the activity of a gene product conferring the activity of “H/ACA ribonucleoprotein complex subunit 3”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Yhr072w-a, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Yhr072w-a, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Yhr072w-a, or a functional equivalent or a homolog thereof as depicted in column 8 of Table II, application no. 6, preferably a homolog or functional equivalent as depicted in column 8 of Table II B, application no. 6, and being depicted in the same respective line as said Yhr072w-a, and preferably the activity is increased non-targeted, whereby the respective line discloses in column 7 the fine chemical isoleucine.

Accordingly, in one embodiment, the molecule which activity is to be increased in the process of the invention is the gene product with an activity as a “H/ACA ribonucleoprotein complex subunit 3”, preferably it is the molecule of section (a) or (b) of this paragraph.

In particular, it was observed that in plants, especially in Arabidopsis thaliana, increasing or generating the activity of a gene product non-targeted with the activity of a “H/ACA ribonucleoprotein complex subunit 3”, preferably being encoded by a gene comprising the nucleic acid sequence SEQ ID NO. 64470, preferably the coding region thereof, conferred the production of or the increase in isoleucine compared with the wild type control.

The nucleic acid sequence of Yhr072w-a from Saccharomyces cerevisiae, e.g. as shown in the respective line in column 5 of Table I, application no. 6, has been published in A. And the activity of the gene product thereof is the activity of H/ACA ribonucleoprotein complex subunit 3.

Accordingly, in one embodiment, the process of the present invention for producing leucine in a non-human organism, like a microorganism or a plant or a part thereof, comprises increasing or generating the activity of a gene product with the activity of a gene product conferring the activity of “H/ACA ribonucleoprotein complex subunit 3”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said Yhr072w-a, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Yhr072w-a, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Yhr072w-a, or a functional equivalent or a homolog thereof as depicted in column 8 of Table II, application no. 6, preferably a homolog or functional equivalent as depicted in column 8 of Table II B, application no. 6, and being depicted in the same respective line as said Yhr072w-a, and preferably the activity is increased non-targeted, whereby the respective line discloses in column 7 the fine chemical leucine.

Accordingly, in one embodiment, the molecule which activity is to be increased in the process of the invention is the gene product with an activity as a “H/ACA ribonucleoprotein complex subunit 3”, preferably it is the molecule of section (a) or (b) of this paragraph.

In particular, it was observed that in plants, especially in Arabidopsis thaliana, increasing or generating the activity of a gene product non-targeted with the activity of a “H/ACA ribonucleoprotein complex subunit 3”, preferably being encoded by a gene comprising the nucleic acid sequence SEQ ID NO. 64470, preferably the coding region thereof, conferred the production of or the increase in leucine compared with the wild type control.

The nucleic acid sequence of Yil155c from Saccharomyces cerevisiae, e.g. as shown in the respective line in column 5 of Table I, application no. 6, has been published in A. And the activity of the gene product thereof is the activity of glycerol-3-phosphate dehydrogenase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “glycerol-3-phosphate dehydrogenase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Yil155c, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Yil155c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Yil155c, or a functional equivalent or a homolog thereof as depicted in column 8 of Table II, application no. 6, preferably a homolog or functional equivalent as depicted in column 8 of Table II B, application no. 6, and being depicted in the same respective line as said Yil155c, and preferably the activity is increased non-targeted, whereby the respective line discloses in column 7 the fine chemical isoleucine.

Accordingly, in one embodiment, the molecule which activity is to be increased in the process of the invention is the gene product with an activity as a “glycerol-3-phosphate dehydrogenase”, preferably it is the molecule of section (a) or (b) of this paragraph.

In particular, it was observed that in plants, especially in Arabidopsis thaliana, increasing or generating the activity of a gene product non-targeted with the activity of a “glycerol-3-phosphate dehydrogenase”, preferably being encoded by a gene comprising the nucleic acid sequence SEQ ID NO. 108514, preferably the coding region thereof, conferred the production of or the increase in isoleucine compared with the wild type control.

The nucleic acid sequence of Yil155c from Saccharomyces cerevisiae, e.g. as shown in the respective line in column 5 of Table I, application no. 6, has been published in A. And the activity of the gene product thereof is the activity of glycerol-3-phosphate dehydrogenase.

Accordingly, in one embodiment, the process of the present invention for producing leucine in a non-human organism, like a microorganism or a plant or a part thereof, comprises increasing or generating the activity of a gene product with the activity of a gene product conferring the activity of “glycerol-3-phosphate dehydrogenase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said Yil155c, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Yil155c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Yil155c, or a functional equivalent or a homolog thereof as depicted in column 8 of Table II, application no. 6, preferably a homolog or functional equivalent as depicted in column 8 of Table II B, application no. 6, and being depicted in the same respective line as said Yil155c, and preferably the activity is increased non-targeted, whereby the respective line discloses in column 7 the fine chemical leucine.

Accordingly, in one embodiment, the molecule which activity is to be increased in the process of the invention is the gene product with an activity as a “glycerol-3-phosphate dehydrogenase”, preferably it is the molecule of section (a) or (b) of this paragraph.

In particular, it was observed that in plants, especially in Arabidopsis thaliana, increasing or generating the activity of a gene product non-targeted with the activity of a “glycerol-3-phosphate dehydrogenase”, preferably being encoded by a gene comprising the nucleic acid sequence SEQ ID NO. 108514, preferably the coding region thereof, conferred the production of or the increase in leucine compared with the wild type control.

The nucleic acid sequence of Yjr139c from Saccharomyces cerevisiae, e.g. as shown in the respective line in column 5 of Table I, application no. 6, has been published in A. And the activity of the gene product thereof is the activity of homoserine dehydrogenase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “homoserine dehydrogenase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Yjr139c, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Yjr139c, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Yjr139c, or a functional equivalent or a homolog thereof as depicted in column 8 of Table II, application no. 6, preferably a homolog or functional equivalent as depicted in column 8 of Table II B, application no. 6, and being depicted in the same respective line as said Yjr139c, and preferably the activity is increased plastidic, whereby the respective line discloses in column 7 the fine chemical isoleucine.

Accordingly, in one embodiment, the molecule which activity is to be increased in the process of the invention is the gene product with an activity as a “homoserine dehydrogenase”, preferably it is the molecule of section (a) or (b) of this paragraph.

In particular, it was observed that in plants, especially in Arabidopsis thaliana, increasing or generating the activity of a gene product plastidic with the activity of a “homoserine dehydrogenase”, preferably being encoded by a gene comprising the nucleic acid sequence SEQ ID NO. 14885, preferably the coding region thereof, conferred the production of or the increase in isoleucine compared with the wild type control.

The nucleic acid sequence of Yjr148w from Saccharomyces cerevisiae, e.g. as shown in the respective line in column 5 of Table I, application no. 6, has been published in A. And the activity of the gene product thereof is the activity of branched chain aminotransferase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “branched chain aminotransferase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Yjr148w, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Yjr148w, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Yjr148w, or a functional equivalent or a homolog thereof as depicted in column 8 of Table II, application no. 6, preferably a homolog or functional equivalent as depicted in column 8 of Table II B, application no. 6, and being depicted in the same respective line as said Yjr148w, and preferably the activity is increased plastidic, whereby the respective line discloses in column 7 the fine chemical isoleucine.

Accordingly, in one embodiment, the molecule which activity is to be increased in the process of the invention is the gene product with an activity as a “branched chain aminotransferase”, preferably it is the molecule of section (a) or (b) of this paragraph.

In particular, it was observed that in plants, especially in Arabidopsis thaliana, increasing or generating the activity of a gene product plastidic with the activity of a “branched chain aminotransferase”, preferably being encoded by a gene comprising the nucleic acid sequence SEQ ID NO. 90409, preferably the coding region thereof, conferred the production of or the increase in isoleucine compared with the wild type control.

The nucleic acid sequence of Yjr148w from Saccharomyces cerevisiae, e.g. as shown in the respective line in column 5 of Table I, application no. 6, has been published in A. And the activity of the gene product thereof is the activity of branched chain aminotransferase.

Accordingly, in one embodiment, the process of the present invention for producing valine in a non-human organism, like a microorganism or a plant or a part thereof, comprises increasing or generating the activity of a gene product with the activity of a gene product conferring the activity of “branched chain aminotransferase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said Yjr148w, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Yjr148w, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Yjr148w, or a functional equivalent or a homolog thereof as depicted in column 8 of Table II, application no. 6, preferably a homolog or functional equivalent as depicted in column 8 of Table II B, application no. 6, and being depicted in the same respective line as said Yjr148w, and preferably the activity is increased plastidic, whereby the respective line discloses in column 7 the fine chemical valine.

Accordingly, in one embodiment, the molecule which activity is to be increased in the process of the invention is the gene product with an activity as a “branched chain aminotransferase”, preferably it is the molecule of section (a) or (b) of this paragraph.

In particular, it was observed that in plants, especially in Arabidopsis thaliana, increasing or generating the activity of a gene product plastidic with the activity of a “branched chain aminotransferase”, preferably being encoded by a gene comprising the nucleic acid sequence SEQ ID NO. 90409, preferably the coding region thereof, conferred the production of or the increase in valine compared with the wild type control.

The nucleic acid sequence of Yjr153w from Saccharomyces cerevisiae, e.g. as shown in the respective line in column 5 of Table I, application no. 6, has been published in A. And the activity of the gene product thereof is the activity of polygalacturonase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “polygalacturonase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Yjr153w, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Yjr153w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Yjr153w, or a functional equivalent or a homolog thereof as depicted in column 8 of Table II, application no. 6, preferably a homolog or functional equivalent as depicted in column 8 of Table II B, application no. 6, and being depicted in the same respective line as said Yjr153w, and preferably the activity is increased non-targeted, whereby the respective line discloses in column 7 the fine chemical isoleucine.

Accordingly, in one embodiment, the molecule which activity is to be increased in the process of the invention is the gene product with an activity as a “polygalacturonase”, preferably it is the molecule of section (a) or (b) of this paragraph.

In particular, it was observed that in plants, especially in Arabidopsis thaliana, increasing or generating the activity of a gene product non-targeted with the activity of a “polygalacturonase”, preferably being encoded by a gene comprising the nucleic acid sequence SEQ ID NO. 66274, preferably the coding region thereof, conferred the production of or the increase in isoleucine compared with the wild type control.

The nucleic acid sequence of Yjr153w from Saccharomyces cerevisiae, e.g. as shown in the respective line in column 5 of Table I, application no. 6, has been published in A. And the activity of the gene product thereof is the activity of polygalacturonase.

Accordingly, in one embodiment, the process of the present invention for producing leucine in a non-human organism, like a microorganism or a plant or a part thereof, comprises increasing or generating the activity of a gene product with the activity of a gene product conferring the activity of “polygalacturonase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said Yjr153w, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Yjr153w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Yjr153w, or a functional equivalent or a homolog thereof as depicted in column 8 of Table II, application no. 6, preferably a homolog or functional equivalent as depicted in column 8 of Table II B, application no. 6, and being depicted in the same respective line as said Yjr153w, and preferably the activity is increased non-targeted, whereby the respective line discloses in column 7 the fine chemical leucine.

Accordingly, in one embodiment, the molecule which activity is to be increased in the process of the invention is the gene product with an activity as a “polygalacturonase”, preferably it is the molecule of section (a) or (b) of this paragraph.

In particular, it was observed that in plants, especially in Arabidopsis thaliana, increasing or generating the activity of a gene product non-targeted with the activity of a “polygalacturonase”, preferably being encoded by a gene comprising the nucleic acid sequence SEQ ID NO. 66274, preferably the coding region thereof, conferred the production of or the increase in leucine compared with the wild type control.

The nucleic acid sequence of Yjr153w from Saccharomyces cerevisiae, e.g. as shown in the respective line in column 5 of Table I, application no. 6, has been published in A. And the activity of the gene product thereof is the activity of polygalacturonase.

Accordingly, in one embodiment, the process of the present invention for producing valine in a non-human organism, like a microorganism or a plant or a part thereof, comprises increasing or generating the activity of a gene product with the activity of a gene product conferring the activity of “polygalacturonase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said Yjr153w, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Yjr153w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Yjr153w, or a functional equivalent or a homolog thereof as depicted in column 8 of Table II, application no. 6, preferably a homolog or functional equivalent as depicted in column 8 of Table II B, application no. 6, and being depicted in the same respective line as said Yjr153w, and preferably the activity is increased non-targeted, whereby the respective line discloses in column 7 the fine chemical valine.

Accordingly, in one embodiment, the molecule which activity is to be increased in the process of the invention is the gene product with an activity as a “polygalacturonase”, preferably it is the molecule of section (a) or (b) of this paragraph.

In particular, it was observed that in plants, especially in Arabidopsis thaliana, increasing or generating the activity of a gene product non-targeted with the activity of a “polygalacturonase”, preferably being encoded by a gene comprising the nucleic acid sequence SEQ ID NO. 66274, preferably the coding region thereof, conferred the production of or the increase in valine compared with the wild type control.

The nucleic acid sequence of YKL038W from Saccharomyces cerevisiae, e.g. as shown in the respective line in column 5 of Table I, application no. 6, has been published in A. And the activity of the gene product thereof is the activity of YKL038W-protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “YKL038W-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said YKL038W, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said YKL038W, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said YKL038W, or a functional equivalent or a homolog thereof as depicted in column 8 of Table II, application no. 6, preferably a homolog or functional equivalent as depicted in column 8 of Table II B, application no. 6, and being depicted in the same respective line as said YKL038W, and preferably the activity is increased plastidic, whereby the respective line discloses in column 7 the fine chemical isoleucine.

Accordingly, in one embodiment, the molecule which activity is to be increased in the process of the invention is the gene product with an activity as a “YKL038W-protein”, preferably it is the molecule of section (a) or (b) of this paragraph.

In particular, it was observed that in plants, especially in Arabidopsis thaliana, increasing or generating the activity of a gene product plastidic with the activity of a “YKL038W-protein”, preferably being encoded by a gene comprising the nucleic acid sequence SEQ ID NO. 90602, preferably the coding region thereof, conferred the production of or the increase in isoleucine compared with the wild type control.

The nucleic acid sequence of YKL038W from Saccharomyces cerevisiae, e.g. as shown in the respective line in column 5 of Table I, application no. 6, has been published in A. And the activity of the gene product thereof is the activity of YKL038W-protein.

Accordingly, in one embodiment, the process of the present invention for producing leucine in a non-human organism, like a microorganism or a plant or a part thereof, comprises increasing or generating the activity of a gene product with the activity of a gene product conferring the activity of “YKL038W-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said YKL038W, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said YKL038W, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said YKL038W, or a functional equivalent or a homolog thereof as depicted in column 8 of Table II, application no. 6, preferably a homolog or functional equivalent as depicted in column 8 of Table II B, application no. 6, and being depicted in the same respective line as said YKL038W, and preferably the activity is increased plastidic, whereby the respective line discloses in column 7 the fine chemical leucine.

Accordingly, in one embodiment, the molecule which activity is to be increased in the process of the invention is the gene product with an activity as a “YKL038W-protein”, preferably it is the molecule of section (a) or (b) of this paragraph.

In particular, it was observed that in plants, especially in Arabidopsis thaliana, increasing or generating the activity of a gene product plastidic with the activity of a “YKL038W-protein”, preferably being encoded by a gene comprising the nucleic acid sequence SEQ ID NO. 90602, preferably the coding region thereof, conferred the production of or the increase in leucine compared with the wild type control.

The nucleic acid sequence of Ylr224w from Saccharomyces cerevisiae, e.g. as shown in the respective line in column 5 of Table I, application no. 6, has been published in A. And the activity of the gene product thereof is the activity of YLR224W-protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “YLR224W-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Ylr224w, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Ylr224w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Ylr224w, or a functional equivalent or a homolog thereof as depicted in column 8 of Table II, application no. 6, preferably a homolog or functional equivalent as depicted in column 8 of Table II B, application no. 6, and being depicted in the same respective line as said Ylr224w, and preferably the activity is increased non-targeted, whereby the respective line discloses in column 7 the fine chemical isoleucine.

Accordingly, in one embodiment, the molecule which activity is to be increased in the process of the invention is the gene product with an activity as a “YLR224W-protein”, preferably it is the molecule of section (a) or (b) of this paragraph.

In particular, it was observed that in plants, especially in Arabidopsis thaliana, increasing or generating the activity of a gene product non-targeted with the activity of a “YLR224W-protein”, preferably being encoded by a gene comprising the nucleic acid sequence SEQ ID NO. 108723, preferably the coding region thereof, conferred the production of or the increase in isoleucine compared with the wild type control.

The nucleic acid sequence of Ylr348c from Saccharomyces cerevisiae, e.g. as shown in the respective line in column 5 of Table I, application no. 6, has been published in A. And the activity of the gene product thereof is the activity of mitochondrial dicarboxylate transporter.

Accordingly, in one embodiment, the process of the present invention for producing isoleucine in a non-human organism, like a microorganism or a plant or a part thereof, comprises increasing or generating the activity of a gene product with the activity of a gene product conferring the activity of “mitochondrial dicarboxylate transporter”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Ylr348c, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Ylr348c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Ylr348c, or a functional equivalent or a homolog thereof as depicted in column 8 of Table II, application no. 6, preferably a homolog or functional equivalent as depicted in column 8 of Table II B, application no. 6, and being depicted in the same respective line as said Ylr348c, and preferably the activity is increased non-targeted, whereby the respective line discloses in column 7 the fine chemical isoleucine.

Accordingly, in one embodiment, the molecule which activity is to be increased in the process of the invention is the gene product with an activity as a “mitochondrial dicarboxylate transporter”, preferably it is the molecule of section (a) or (b) of this paragraph.

In particular, it was observed that in plants, especially in Arabidopsis thaliana, increasing or generating the activity of a gene product non-targeted with the activity of a “mitochondrial dicarboxylate transporter”, preferably being encoded by a gene comprising the nucleic acid sequence SEQ ID NO. 98243, preferably the coding region thereof, conferred the production of or the increase in isoleucine compared with the wild type control.

The nucleic acid sequence of Ylr359w from Saccharomyces cerevisiae, e.g. as shown in the respective line in column 5 of Table I, application no. 6, has been published in A. And the activity of the gene product thereof is the activity of adenylosuccinate lyase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “adenylosuccinate lyase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Ylr359w, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Ylr359w, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Ylr359w, or a functional equivalent or a homolog thereof as depicted in column 8 of Table II, application no. 6, preferably a homolog or functional equivalent as depicted in column 8 of Table II B, application no. 6, and being depicted in the same respective line as said Ylr359w, and preferably the activity is increased plastidic, whereby the respective line discloses in column 7 the fine chemical isoleucine.

Accordingly, in one embodiment, the molecule which activity is to be increased in the process of the invention is the gene product with an activity as a “adenylosuccinate lyase”, preferably it is the molecule of section (a) or (b) of this paragraph.

In particular, it was observed that in plants, especially in Arabidopsis thaliana, increasing or generating the activity of a gene product plastidic with the activity of a “adenylosuccinate lyase”, preferably being encoded by a gene comprising the nucleic acid sequence SEQ ID NO. 67190, preferably the coding region thereof, conferred the production of or the increase in isoleucine compared with the wild type control.

The nucleic acid sequence of Ylr359w from Saccharomyces cerevisiae, e.g. as shown in the respective line in column 5 of Table I, application no. 6, has been published in A. And the activity of the gene product thereof is the activity of adenylosuccinate lyase.

Accordingly, in one embodiment, the process of the present invention for producing leucine in a non-human organism, like a microorganism or a plant or a part thereof, comprises increasing or generating the activity of a gene product with the activity of a gene product conferring the activity of “adenylosuccinate lyase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said Ylr359w, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Ylr359w, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Ylr359w, or a functional equivalent or a homolog thereof as depicted in column 8 of Table II, application no. 6, preferably a homolog or functional equivalent as depicted in column 8 of Table II B, application no. 6, and being depicted in the same respective line as said Ylr359w, and preferably the activity is increased plastidic, whereby the respective line discloses in column 7 the fine chemical leucine.

Accordingly, in one embodiment, the molecule which activity is to be increased in the process of the invention is the gene product with an activity as a “adenylosuccinate lyase”, preferably it is the molecule of section (a) or (b) of this paragraph.

In particular, it was observed that in plants, especially in Arabidopsis thaliana, increasing or generating the activity of a gene product plastidic with the activity of a “adenylosuccinate lyase”, preferably being encoded by a gene comprising the nucleic acid sequence SEQ ID NO. 67190, preferably the coding region thereof, conferred the production of or the increase in leucine compared with the wild type control.

The nucleic acid sequence of Ylr359w from Saccharomyces cerevisiae, e.g. as shown in the respective line in column 5 of Table I, application no. 6, has been published in A. And the activity of the gene product thereof is the activity of adenylosuccinate lyase.

Accordingly, in one embodiment, the process of the present invention for producing valine in a non-human organism, like a microorganism or a plant or a part thereof, comprises increasing or generating the activity of a gene product with the activity of a gene product conferring the activity of “adenylosuccinate lyase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said Ylr359w, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Ylr359w, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Ylr359w, or a functional equivalent or a homolog thereof as depicted in column 8 of Table II, application no. 6, preferably a homolog or functional equivalent as depicted in column 8 of Table II B, application no. 6, and being depicted in the same respective line as said Ylr359w, and preferably the activity is increased plastidic, whereby the respective line discloses in column 7 the fine chemical valine.

Accordingly, in one embodiment, the molecule which activity is to be increased in the process of the invention is the gene product with an activity as a “adenylosuccinate lyase”, preferably it is the molecule of section (a) or (b) of this paragraph.

In particular, it was observed that in plants, especially in Arabidopsis thaliana, increasing or generating the activity of a gene product plastidic with the activity of a “adenylosuccinate lyase”, preferably being encoded by a gene comprising the nucleic acid sequence SEQ ID NO. 67190, preferably the coding region thereof, conferred the production of or the increase in valine compared with the wild type control.

The nucleic acid sequence of Yml123c from Saccharomyces cerevisiae, e.g. as shown in the respective line in column 5 of Table I, application no. 6, has been published in A. And the activity of the gene product thereof is the activity of phosphate transporter.

Accordingly, in one embodiment, the process of the present invention for producing leucine in a non-human organism, like a microorganism or a plant or a part thereof, comprises increasing or generating the activity of a gene product with the activity of a gene product conferring the activity of “phosphate transporter”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said Yml123c, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Yml123c, and preferably the activity is increased plastidic, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Yml123c, or a functional equivalent or a homolog thereof as depicted in column 8 of Table II, application no. 6, preferably a homolog or functional equivalent as depicted in column 8 of Table II B, application no. 6, and being depicted in the same respective line as said Yml123c, and preferably the activity is increased plastidic, whereby the respective line discloses in column 7 the fine chemical leucine.

Accordingly, in one embodiment, the molecule which activity is to be increased in the process of the invention is the gene product with an activity as a “phosphate transporter”, preferably it is the molecule of section (a) or (b) of this paragraph.

In particular, it was observed that in plants, especially in Arabidopsis thaliana, increasing or generating the activity of a gene product plastidic with the activity of a “phosphate transporter”, preferably being encoded by a gene comprising the nucleic acid sequence SEQ ID NO. 91785, preferably the coding region thereof, conferred the production of or the increase in leucine compared with the wild type control.

The nucleic acid sequence of Ypl028w from Saccharomyces cerevisiae, e.g. as shown in the respective line in column 5 of Table I, application no. 6, has been published in A. And the activity of the gene product thereof is the activity of acetyl-CoA acetyltransferase.

Accordingly, in one embodiment, the process of the present invention for producing isoleucine in a non-human organism, like a microorganism or a plant or a part thereof, comprises increasing or generating the activity of a gene product with the activity of a gene product conferring the activity of “acetyl-CoA acetyltransferase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Ypl028w, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Ypl028w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Ypl028w, or a functional equivalent or a homolog thereof as depicted in column 8 of Table II, application no. 6, preferably a homolog or functional equivalent as depicted in column 8 of Table II B, application no. 6, and being depicted in the same respective line as said Ypl028w, and preferably the activity is increased non-targeted, whereby the respective line discloses in column 7 the fine chemical isoleucine.

Accordingly, in one embodiment, the molecule which activity is to be increased in the process of the invention is the gene product with an activity as a “acetyl-CoA acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyl-CoA acetyltransferase”, preferably being encoded by a gene comprising the nucleic acid sequence SEQ ID NO. 92668, preferably the coding region thereof, conferred the production of or the increase in isoleucine compared with the wild type control.

The nucleic acid sequence of Ypl028w from Saccharomyces cerevisiae, e.g. as shown in the respective line in column 5 of Table I, application no. 6, has been published in A. And the activity of the gene product thereof is the activity of acetyl-CoA acetyltransferase.

Accordingly, in one embodiment, the process of the present invention for producing leucine in a non-human organism, like a microorganism or a plant or a part thereof, comprises increasing or generating the activity of a gene product with the activity of a gene product conferring the activity of “acetyl-CoA acetyltransferase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said Ypl028w, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Ypl028w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Ypl028w, or a functional equivalent or a homolog thereof as depicted in column 8 of Table II, application no. 6, preferably a homolog or functional equivalent as depicted in column 8 of Table II B, application no. 6, and being depicted in the same respective line as said Ypl028w, and preferably the activity is increased non-targeted, whereby the respective line discloses in column 7 the fine chemical leucine.

Accordingly, in one embodiment, the molecule which activity is to be increased in the process of the invention is the gene product with an activity as a “acetyl-CoA acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyl-CoA acetyltransferase”, preferably being encoded by a gene comprising the nucleic acid sequence SEQ ID NO. 92668, preferably the coding region thereof, conferred the production of or the increase in leucine compared with the wild type control.

The nucleic acid sequence of Ypr119w from Saccharomyces cerevisiae, e.g. as shown in the respective line in column 5 of Table I, application no. 6, has been published in A. And the activity of the gene product thereof is the activity of cyclin.

Accordingly, in one embodiment, the process of the present invention for producing isoleucine in a non-human organism, like a microorganism or a plant or a part thereof, comprises increasing or generating the activity of a gene product with the activity of a gene product conferring the activity of “cyclin”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Ypr119w, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Ypr119w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Ypr119w, or a functional equivalent or a homolog thereof as depicted in column 8 of Table II, application no. 6, preferably a homolog or functional equivalent as depicted in column 8 of Table II B, application no. 6, and being depicted in the same respective line as said Ypr119w, and preferably the activity is increased non-targeted, whereby the respective line discloses in column 7 the fine chemical isoleucine.

Accordingly, in one embodiment, the molecule which activity is to be increased in the process of the invention is the gene product with an activity as a “cyclin”, preferably it is the molecule of section (a) or (b) of this paragraph.

In particular, it was observed that in plants, especially in Arabidopsis thaliana, increasing or generating the activity of a gene product non-targeted with the activity of a “cyclin”, preferably being encoded by a gene comprising the nucleic acid sequence SEQ ID NO. 98445, preferably the coding region thereof, conferred the production of or the increase in isoleucine compared with the wild type control.

The nucleic acid sequence of Zm_{—}4842_BE510522 from Zea mays, e.g. as shown in the respective line in column 5 of Table I, application no. 6, is unpublished. And the activity of the gene product thereof is the activity of Zm_{—}4842_BE510522-protein.

Accordingly, in one embodiment, the process of the present invention for producing isoleucine in a non-human organism, like a microorganism or a plant or a part thereof, comprises increasing or generating the activity of a gene product with the activity of a gene product conferring the activity of “Zm_{—}4842_BE510522-protein”, especially from Zea mays or its functional equivalent or its homolog, e.g. the increase of
(a) a gene product of a gene comprising the nucleic acid molecule as shown in the respective line in column 5 of Table I (whereby the respective line discloses in column 7 the fine chemical isoleucine), application no. 6, preferably the coding region thereof, or a homolog or a fragment thereof, and being depicted in the same respective line as said Zm_{—}4842_BE510522, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Zm_{—}4842_BE510522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Zm_{—}4842_BE510522, or a functional equivalent or a homolog thereof as depicted in column 8 of Table II, application no. 6, preferably a homolog or functional equivalent as depicted in column 8 of Table II B, application no. 6, and being depicted in the same respective line as said Zm_{—}4842_BE510522, and preferably the activity is increased non-targeted, whereby the respective line discloses in column 7 the fine chemical isoleucine.

Accordingly, in one embodiment, the molecule which activity is to be increased in the process of the invention is the gene product with an activity as a “Zm_{—}4842_BE510522-protein”, preferably it is the molecule of section (a) or (b) of this paragraph.

In particular, it was observed that in plants, especially in Arabidopsis thaliana, increasing or generating the activity of a gene product non-targeted with the activity of a

“Zm_{—}4842_BE510522-protein”, preferably being encoded by a gene comprising the nucleic acid sequence SEQ ID NO. 68413, preferably the coding region thereof, conferred the production of or the increase in isoleucine compared with the wild type control.

The nucleic acid sequence of Zm_{—}4842_BE510522 from Zea mays, e.g. as shown in the respective line in column 5 of Table I, application no. 6, is unpublished. And the activity of the gene product thereof is the activity of Zm_{—}4842_BE510522-protein.

Accordingly, in one embodiment, the process of the present invention for producing leucine in a non-human organism, like a microorganism or a plant or a part thereof, comprises increasing or generating the activity of a gene product with the activity of a gene product conferring the activity of “Zm_{—}4842_BE510522-protein”, especially from Zea mays or its functional equivalent or its homolog, e.g. the increase of
(a) a gene product of a gene comprising the nucleic acid molecule as shown in the respective line in column 5 of Table I (whereby the respective line discloses in column 7 the fine chemical leucine), application no. 6, preferably the coding region thereof, or a homolog or a fragment thereof, and being depicted in the same respective line as said Zm_{—}4842_BE510522, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Zm_{—}4842_BE510522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Zm_{—}4842_BE510522, or a functional equivalent or a homolog thereof as depicted in column 8 of Table II, application no. 6, preferably a homolog or functional equivalent as depicted in column 8 of Table II B, application no. 6, and being depicted in the same respective line as said Zm_{—}4842_BE510522, and preferably the activity is increased non-targeted, whereby the respective line discloses in column 7 the fine chemical leucine.

Accordingly, in one embodiment, the molecule which activity is to be increased in the process of the invention is the gene product with an activity as a “Zm_{—}4842_BE510522-protein”, preferably it is the molecule of section (a) or (b) of this paragraph.

In particular, it was observed that in plants, especially in Arabidopsis thaliana, increasing or generating the activity of a gene product non-targeted with the activity of a “Zm_{—}4842_BE510522-protein”, preferably being encoded by a gene comprising the nucleic acid sequence SEQ ID NO. 68413, preferably the coding region thereof, conferred the production of or the increase in leucine compared with the wild type control.

The nucleic acid sequence of Zm_{—}4842_BE510522 from Zea mays, e.g. as shown in the respective line in column 5 of Table I, application no. 6, is unpublished. And the activity of the gene product thereof is the activity of Zm_{—}4842_BE510522-protein.

Accordingly, in one embodiment, the process of the present invention for producing valine in a non-human organism, like a microorganism or a plant or a part thereof, comprises increasing or generating the activity of a gene product with the activity of a gene product conferring the activity of “Zm_{—}4842_BE510522-protein”, especially from Zea mays or its functional equivalent or its homolog, e.g. the increase of
(a) a gene product of a gene comprising the nucleic acid molecule as shown in the respective line in column 5 of Table I (whereby the respective line discloses in column 7 the fine chemical valine), application no. 6, preferably the coding region thereof, or a homolog or a fragment thereof, and being depicted in the same respective line as said Zm_{—}4842_BE510522, or a functional equivalent or a homolog thereof as shown in column 8 of Table I, application no. 6, preferably the coding region thereof, particularly a homolog or functional equivalent as shown in column 8 of Table I B, application no. 6, and being depicted in the same respective line as said Zm_{—}4842_BE510522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 6, respectively, and being depicted in the same respective line as said Zm_{—}4842_BE510522, or a functional equivalent or a homolog thereof as depicted in column 8 of Table II, application no. 6, preferably a homolog or functional equivalent as depicted in column 8 of Table II B, application no. 6, and being depicted in the same respective line as said Zm_{—}4842_BE510522, and preferably the activity is increased non-targeted, whereby the respective line discloses in column 7 the fine chemical valine.

Accordingly, in one embodiment, the molecule which activity is to be increased in the process of the invention is the gene product with an activity as a “Zm_{—}4842_BE510522-protein”, preferably it is the molecule of section (a) or (b) of this paragraph.

In particular, it was observed that in plants, especially in Arabidopsis thaliana, increasing or generating the activity of a gene product non-targeted with the activity of a “Zm_{—}4842_BE510522-protein”, preferably being encoded by a gene comprising the nucleic acid sequence SEQ ID NO. 68413, preferably the coding region thereof, conferred the production of or the increase in valine compared with the wild type control.

[0105.1.1.6] to [0107.1.1.6] for the disclosure of these paragraphs see [0105.1.1.1] to [0107.1.1.1] above.

A protein having an activity conferring an increase in the amount or level of the fine chemical isoleucine, leucine, or valine, upon targeting to the plastids or mitochondria or upon non-targeting, preferably has the structure of the respective polypeptide described herein, in particular of the polypeptides comprising the consensus sequence or at least one polypeptide motifs as shown in the respective line in Table IV, application no. 6, column 8 or of the polypeptide comprising an amino acid sequence as disclosed in the respective line in Table II, application no. 6, columns 5 or 8, or homologs or fragments thereof as described herein, or is encoded by the nucleic acid molecule characterized herein or the nucleic acid molecule according to the invention, for example by the nucleic acid molecule as shown in the respective line in Table I, application no. 6, columns 5 or 8, preferably the coding region thereof, or homologs or fragments thereof and has the herein mentioned activity.

[0109.1.1.6] to [0110.1.1.6] for the disclosure of these paragraphs see [0109.1.1.1] to [0110.1.1.1] above.

In one embodiment, the process of the present invention comprises one or more of the following steps
(a) stabilizing a protein conferring the generation or increased expression of a protein encoded by the nucleic acid molecule of the invention or of the polypeptide of the invention, e.g. of a polypeptide having the activity selected from the group consisting of 2,3-dihydroxyphenylpropionate 1,2-dioxygenase, 2-isopropylmalate synthase, 3-isopropylmalate dehydrogenase, 47266012-protein, 50S ribosomal protein L14, ABC transporter ATP binding component, ABC transporter ATP-binding protein, ABC transporter component, ABC transporter permease protein, acetolactate synthase, acetolactate synthase small subunit, acetyl CoA carboxylase, acetyl-CoA acetyltransferase, acetyltransferase, acid shock protein, acyl transferase, acyltransferase, adenylate kinase, adenylosuccinate lyase, agmatinase, aldehyde dehydrogenase, aminotransferase, anthranilate synthase component II, arginine exporter protein, At1g19800-protein, At1g23110-protein, At1g47380-protein, At4g32480-protein, At5g16650-protein, ATP-binding component of a transport system, auxin response factor, b0050-protein, b1003-protein, b1163-protein, b1445-protein, b1522-protein, b1670-protein, b1672-protein, b2513-protein, b2613-protein, b2673-protein, b2739-protein, b2812-protein, b3083-protein, b3490-protein, b3989-protein, b4029-protein, b4050-protein, b4056-protein, b4263-protein, betagalactosidase, beta-hydroxylase, bifunctional protein (phosphoribosyltransferase and regulatory protein), binding-protein-dependent transport systems inner membrane component, branched chain aminotransferase, branched-chain amino acid permease, branched-chain amino-acid aminotransferase, calcium-dependent protein kinase, carbamoyl-phosphate synthase subunit, CBL-interacting protein kinase, cell division control protein, chaperone protein CIpB, cullin, cyclin, cyclin D, cytochrome c-type protein, dihydroxyacid dehydratase, disease resistance protein, DNA helicase II, DNA helicase IV, DNA mismatch repair protein, DNA polymerase, DNA-binding protein, elongation factor Tu, enoyl-CoA hydratase, eukaryotic translation initiation factor, eukaryotic translation initiation factor 5, ferredoxin, flavodoxin, fumarylacetoacetate hydrolase, GDP-mannose 4,6-dehydratase, geranylgeranyl pyrophosphate synthase, gluconate transport system permease 3, glucose-1-phosphate cytidylyltransferase, glucose-6-phosphate 1-dehydrogenase, glutamate-ammonia-ligase, glutamine synthetase, glutaredoxin, glycerol 3-phosphate dehydrogenase, glycogen (starch) synthase, glycogen synthase, glycoprotease, glycosyl transferase, GM02LC11114-protein, GM02LC5744-protein, H/ACA ribonucleoprotein complex subunit 3, harpin-induced family protein, heat shock transcription factor, hexose transporter, histone H2A, homocitrate synthase, homoserine dehydrogenase, hydrolase, iron(III) dicitrate-binding protein, isochorismate synthase, isopropylmalate isomerase large subunit, ketol-acid reductoisomerase, L-serine dehydratase, MADS-box transcription factor, major facilitator superfamily transporter protein, malate dehydrogenase, malic enzyme, membrane protein, membrane transport protein, methylglyoxal synthase, mitochondrial dicarboxylate transporter, monothiol glutaredoxin, murein transglycosylase, Na+/K+ transporter, NAD(P)H-quinone oxidoreductase subunit, NADH-quinone oxidoreductase subunit, NADP-dependent malic enzyme, Nucleoside monophosphate phosphohydrolase, O-antigen chain length determinant, oxidoreductase, oxireductase, peptidase, phosphate transporter, phosphoadenosine phosphosulfate reductase, phosphoanhydride phosphorylase, phosphoglycerate kinase, phosphopantetheine adenylyltransferase, photosystem II protein, polygalacturonase, polyphosphate kinase, precorrin methylase, protein kinase, protein phosphatase, protoporphyrin oxidase, purine nucleoside phosphorylase, purine-nucleoside phosphorylase, pyruvate kinase, riboflavin biosynthesis protein, ribosephosphate isomerase, constitutive, RNA binding protein, RNA-binding protein, Sec-independent protein translocase subunit, serine hydroxymethyltransferase, short-chain alcohol dehydrogenase family, sll1032-protein, sll1455-protein, sll1761-protein, superoxide dismutase, thioredoxin, thioredoxin family protein, threonine dehydratase, threonine synthase, transcription factor, transcriptional regulator, transport protein, trehalose-phosphatase, TTC1386-protein, urease subunit, vacuolar ATPase subunit, valine-pyruvate transaminase, YAL013W-protein, ydr183w-protein, ygl237c-protein, YGR193C-protein, yhr006w-protein, YKL038W-protein, YLR224W-protein, zinc finger protein, zinc transporter, and Zm_{—}4842_BE510522-protein, or of a polypeptide as indicated in the respective line in Table II, application no. 6, columns 5 or 8, or its homologs or fragments, and conferring the production of or an increase in isoleucine, leucine, or valine, respectively; and/or
(b) stabilizing a mRNA conferring the generation or increased expression of a FCRP, e.g. protein encoded by the nucleic acid molecule of the invention or its homologs or fragments, or of a mRNA encoding the polypeptide of the present invention having the hereinmentioned activity selected from the group consisting of said activities mentioned in (a) and conferring the production of or an increase in isoleucine, leucine, or valine, respectively; and/or
(c) increasing the specific activity of a protein conferring the increased expression of a FCRP, e.g. a protein encoded by the nucleic acid molecule of the invention or of the polypeptide of the present invention having herein-mentioned isoleucine, leucine, or valine generating or increasing activity, respectively, e.g. of a polypeptide having the activity of a protein as indicated in the respective line in Table 11, application no. 6, columns 5 or 8, or its homologs or fragments, or decreasing the inhibitory regulation of the polypeptide of the invention; and/or
(d) generating or increasing the expression of an endogenous or artificial transcription factor mediating the expression of a protein conferring the generation or increased expression of a FCRP, e.g. a protein encoded by the nucleic acid molecule of the invention or of the polypeptide of the invention having herein-mentioned activity selected from the group consisting of said activities mentioned in (a) and conferring a isoleucine, leucine, or valine increasing activity, respectively, e.g. of a polypeptide having the activity of a protein as indicated in the respective line in Table II, application no. 6, columns 5 or 8 or its homologs; and/or
(e) stimulating activity of a protein conferring the increased expression of a FCRP, e.g. a protein encoded by the nucleic acid molecule of the present invention or a polypeptide of the present invention having herein-mentioned activity selected from the group consisting of said activities mentioned in (a) and conferring a isoleucine, leucine, or valine increasing activity, respectively, e.g. of a polypeptide having the activity of a protein as indicated in the respective line in Table II, application no. 6, columns 5 or 8, or its homologs or fragments, by adding one or more exogenous inducing factors to the non-human organism or parts thereof; and/or
(f) expressing a transgenic gene encoding a protein conferring the increased expression of a FCRP, e.g. a polypeptide encoded by the nucleic acid molecule of the present invention or a polypeptide of the present invention, having herein-mentioned activity selected from the group consisting of said activities mentioned in (a) and conferring a isoleucine, leucine, or valine increasing activity, respectively, e.g. of a polypeptide having the activity of a protein as indicated in the respective line in Table II, application no. 6, columns 5 or 8, or its homologs or fragments, and/or
(g) increasing the copy number of a gene conferring the increased expression of a nucleic acid molecule encoding a FCRP, e.g. a polypeptide encoded by the nucleic acid molecule of the invention or the polypeptide of the invention having herein-mentioned activity selected from the group consisting of said activities mentioned in (a) and conferring a isoleucine, leucine, or valine increasing activity, respectively, e.g. of a polypeptide having the activity of a protein as indicated in the respective line in Table II, application no. 6, columns 5 or 8, or its homologs or fragments; and/or
(h) increasing the expression of the endogenous gene encoding the FCRP, polypeptide of the invention, e.g. a polypeptide encoded by the nucleic acid molecule of the invention or the polypeptide of the invention having herein-mentioned activity selected from the group consisting of said activities mentioned in (a) and conferring a isoleucine, leucine, or valine; increasing activity, respectively, e.g. of a polypeptide having the activity of a protein as indicated in the respective line in Table II, application no. 6, columns 5 or 8, or its homologs or fragments, by adding positive expression or removing negative expression elements, e.g. homologous recombination can be used to either introduce positive regulatory elements like for plants the 35S enhancer into the promoter or to remove repressor elements form regulatory regions. Further gene conversion methods can be used to disrupt repressor elements or to enhance to activity of positive elements. Positive elements can be randomly introduced in plants by T-DNA or transposon mutagenesis and lines can be identified in which the positive elements have be integrated near to a gene of the invention, the expression of which is thereby enhanced; and/or
(i) modulating growth conditions of the non-human organism in such a manner, that the expression or activity of the gene encoding the FCRP, e.g. a protein of the invention or the protein itself is enhanced, for example microorganisms or plants can be grown for example under a higher temperature regime leading to an enhanced expression of heat shock proteins, which can lead to an enhanced isoleucine, leucine, or valine production, respectively; and/or
(j) selecting of non-human organisms with especially high activity of the proteins of the invention from natural or from mutagenized resources and breeding them into the target organisms, e.g. the elite crops; and/or
(k) directing a protein encoded by the nucleic acid molecule of the invention or of the polypeptide of the present invention having herein-mentioned activity selected from the group consisting of said activities mentioned in (a) and conferring a isoleucine, leucine, or valine increasing activity, respectively, e.g. of a polypeptide having the activity of a protein as indicated in the respective line in Table II, application no. 6, columns 5 or 8, or its homologs or fragments, if for the corresponding nucleic acid molecule in the respective line in column 6 of Table I the term “plastidic” is indicated, to the plastids by the addition of a plastidial targeting sequence or if for the corresponding nucleic acid molecule in the respective line in column 6 of Table I the term “mitochondria!” is indicated, to the mitochondria by the addition of a mitochondrial targeting sequence; and/or
(l) generating the expression of a protein encoded by the nucleic acid molecule of the invention or of the polypeptide of the present invention having herein-mentioned activity selected from the group consisting of said activities mentioned in (a) and conferring a isoleucine, leucine, or valine increasing activity, respectively, e.g. of a polypeptide having the activity of a protein as indicated in the respective line in Table II, application no. 6, columns 5 or 8, or its homologs or fragments, if for the corresponding nucleic acid molecule in the respective line in column 6 of Table I the term “plastidic” or “mitochondria!” is indicated, in these organelles by the stable or transient transformation, advantageously stable transformation, of organelles, preferably plastids or mitochondria, with an inventive nucleic acid sequence preferably in form of an expression cassette containing said sequence leading to the expression of the nucleic acids or polypeptides of the invention in the respective organelle; and/or
(m) generating the expression of a protein encoded by the nucleic acid molecule of the invention or of the polypeptide of the present invention having herein-mentioned activity selected from the group consisting of said activities mentioned in (a) and conferring a isoleucine, leucine, and/or valine increasing activity, respectively, e.g. of a polypeptide having the activity of a protein as indicated in the respective line in Table II, application no. 6, columns 5 or 8, or its homologs or fragments, if for the corresponding nucleic acid molecu te in the respective line in column 6 of Table I the term “plastidic” or “mitochondria!” is indicated, in these organelles by integration of a nucleic acid of the invention into the genome of the respective organelle under control of preferable a promoter selective for the respective organelle.

Preferably, said mRNA is the coding region of the nucleic acid molecule of the present invention and/or the protein conferring the increased expression of a protein encoded by the coding region of the nucleic acid molecule of the present invention alone or linked to a transit nucleic acid sequence or transit peptide encoding nucleic acid sequence or the polypeptide having the herein mentioned activity, e.g. conferring the generation of or increase of isoleucine, leucine, or valine, respectively, after increasing the expression or activity of the encoded polypeptide, non-targeted or in organelles such as plastids and/or mitochondria, preferably plastids, or having the activity of a polypeptide having an activity as the protein as shown in the respective line in Table II, application no. 6, column 3, or its homologs. Preferably the increase of isoleucine, leucine, or valine, respectively, takes place non-targeted or in plastids and/or mitochondria, preferably non-targeted or in plastids.

[0113.1.1.6] to [0122.1.1.6] for the disclosure of these paragraphs see [0113.1.1.1] to [0122.1.1.1] above.

The activation of an endogenous polypeptide having above-mentioned activity, e.g. having the activity of a protein as shown in the respective line in Table II, application no. 6, column 3 or of the polypeptide of the invention, e.g. conferring the increase of the fine chemical isoleucine, leucine, or valine, respectively, by increase of expression or activity in the cytoplasm, and/or in the cytosol, and/or in an organelle, such as plastids or mitochondria, can also be increased by introducing a synthetic transcription factor, which binds close to the coding region of the gene encoding the protein as shown in the respective line in Table II, application no. 6, column 5 or 8, or homologs or fragments thereof, and activates its transcription. A chimeric zinc finger protein can be constructed, which comprises a specific DNA-binding domain and an activation domain as e.g. the VP16 domain of Herpes Simplex virus. The specific binding domain can bind to the regulatory region of the gene encoding the protein as shown in the respective line in Table II, application no. 6, column 5 or 8, or homologs or fragments thereof. The expression of the chimeric transcription factor in a non-human organism, in particular in a plant, leads to a specific expression of the protein as shown in the respective line in Table II, application no. 6, column 5 or 8, or homologs or fragments thereof, see e.g. in WO01/52620, Oriz, Proc. Natl. Acad. Sci. USA 99, 13290 (2002), or Guan, Proc. Natl. Acad. Sci. USA 99, 13296 (2002).

[0124.1.1.6] to [0127.1.1.6] for the disclosure of these paragraphs see [0124.1.1.1] to [0127.1.1.1] above.

Owing to the introduction of a gene or a plurality of genes conferring the expression of the nucleic acid molecule of the invention or the polypeptide of the invention, for example the nucleic acid construct mentioned below, or encoding the protein as shown in the respective line in Table II, application no. 6, column 5 or 8, or homologs or fragments thereof, into a non-human organism alone or in combination with other genes, it is possible not only to increase the biosynthetic flux towards the end product, but also to increase, modify or create de novo an advantageous, preferably novel metabolites composition in the non-human organism, e.g. an advantageous composition comprising a higher content of (from a viewpoint of nutritional physiology limited) isoleucine, leucine, or valine, respectively, and if desired other amino acids, and/or other metabolites, in free or bound form.

for the disclosure of this paragraph see [0129.1.1.1] above

Accordingly, in one embodiment, the process according to the invention relates to a process, which comprises:
(a) providing a non-human organism, preferably a microorganism, a plant cell, a plant tissue, a plant or a part thereof, more preferably a microorganism, a plant tissue, a plant or a part thereof;
(b) increasing the activity of a protein as shown in the respective line in Table II, application no. 6, column 3 or of a polypeptide being encoded by the respective nucleic acid molecule of the present invention and described below, e.g. conferring an increase of the fine chemical isoleucine, leucine, or valine, respectively, in the non-human organism, preferably in the microorganism, the plant cell, the plant tissue, the plant or a part thereof, more preferably a microorganism, a plant tissue, a plant or a part thereof, especially cytoplasmic or in an organelle, like plastids or mitochondria,
(c) growing the non-human organism, preferably the microorganism, the plant cell, the plant tissue, the plant or a part thereof, under conditions which permit the production of said fine chemical in the non-human organism, preferably the microorganism, the plant cell, the plant tissue, the plant or a part thereof; and
(d) if desired, recovering, optionally isolating, said fine chemical, in free and/or bound form, and, optionally further free and/or bound amino acids, and/or other metabolites synthesized by the non-human organism, the microorganism, the plant cell, the plant tissue, the plant or a part thereof.

[0131.1.1.6] to [0139.1.1.6] for the disclosure of these paragraphs see [0131.1.1.1] to [0139.1.1.6] above.

In another embodiment, the invention relates to a (isolated) nucleic acid molecule, which comprises a nucleic acid molecule selected from the group consisting of:
(a) a nucleic acid molecule encoding a polypeptide shown in Table II, application no. 6, column 5 or 8, preferably shown in Table II B, application no. 6, column 8, or a homolog or a fragment thereof;
(b) a nucleic acid molecule shown in Table I, application no. 6, column 5 or 8, preferably shown in Table I B, application no. 6, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 6, preferably in column 8 of Table II B, application no. 6;
(d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 6, preferably shown in column 8 of Table I B, application no. 6, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 6;
(i) a nucleic acid molecule encoding a polypeptide having the activity represented by a respective protein as depicted in column 5 of Table II, application no. 6;
(j) a nucleic acid molecule comprising a polynucleotide which is obtained by amplifying a cDNA library or a genomic library using the primers in Table III, application no. 6, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j) ;
or a nucleic acid molecule comprising a sequence which is complementary thereto.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 6.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II application no. 6 and confers a generation or an increase of the respective fine chemical in a non-human organism or a part thereof as compared to a corresponding wild-type non-human organism or part thereof.

In another embodiment, the invention relates to a (isolated) nucleic acid molecule, which comprises a nucleic acid molecule selected from the group consisting of:
(a) a nucleic acid molecule encoding a polypeptide shown in Table II, column 5 or 8, application no. 6, preferably shown in Table II A, application no. 6, in column 5 or in Table II A, application no. 6, column 8 or in Table II B, application no. 6, column 8, or a homolog or a fragment thereof;
(b) a nucleic acid molecule shown in Table I, application no. 6, column 5 or 8, preferably shown in Table I A, application no. 6, in column 5 or in Table I A, application no. 6, column 8 or in Table I B, application no. 6, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 6, preferably shown in Table II A, application no. 6, in column 5 or in Table II A, application no. 6, column 8 or in Table II B, application no. 6, column 8;
(d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 6, preferably shown in Table I A, application no. 6, in column 5 or in Table I A, application no. 6, column 8 or in Table I B column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 6;
(i) a nucleic acid molecule encoding a polypeptide having the activity represented by a respective protein as depicted in column 5 of Table II, application no. 6;
(j) a nucleic acid molecule comprising a polynucleotide which is obtained by amplifying a cDNA library or a genomic library using the primers in Table III, application no. 6, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt,500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto;
   whereby, preferably, the nucleic acid molecule according to (a), (b), (c), (d), (e), (f), (g), (h), (i), (j) or (k) distinguishes over the sequence depicted in Table I A and/or I B, application no. 6, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 6, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 6, column 5 or 8, or the coding region thereof, respectively. In a further embodiment the nucleic acid molecule does not encode the polypeptide sequence shown in Table II A and/or II B, application no. 6, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 6, column 5 or 8 and does not encode a protein of the sequence shown in Table II A and/or II B, application no. 6, column 5 or 8.Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 6, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 6, column 5 or 8 but less than 100%, preferably less than 99.999%, 99.99%, 99.9%, 99%, 98%, 97%, 96% or 95% identical to the sequence shown in Table II A and/or II B, application no. 6, column 5 or 8.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 6.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II, application no. 6, and confers a generation or an increase of the respective fine chemical in a non-human organism or a part thereof as compared to a corresponding wild-type non-human organism or part thereof.

[0144.1.1.6] to [0155.1.1.6] for the disclosure of these paragraphs see [0144.1.1.1] to [0155.1.1.1] above.

In a preferred embodiment a nucleic acid construct, for example an expression cassette, comprises upstream, i.e. at the 5′ end of the encoding sequence, a promoter and downstream, i.e. at the 3′ end, a polyadenylation signal and optionally other regulatory elements which are operably linked to the intervening encoding sequence with one of the nucleic acids as depicted in the respective line in Table I, application no. 6, column 5 or 8, preferably the coding region thereof, or a homolog or a fragment thereof. By an operable linkage is meant the sequential arrangement of promoter, encoding sequence, terminator, optionally other regulatory elements and optionally targeting sequences in such a way that each of the regulatory elements/targeting sequence can fulfill its function in the expression of the encoding sequence in due manner. In one embodiment the sequences preferred for operable linkage are targeting sequences for ensuring subcellular localization in organelles, like plastids or mitochondria. However, targeting sequences for ensuring subcellular localization in the endoplasmic reticulum (=ER), in the nucleus, in oil corpuscles or other compartments may also be employed as well as translation promoters such as the 5′ lead sequence in tobacco mosaic virus (Gallie et al., Nucl. Acids Res. 15, 8693 (1987)).

for the disclosure of this paragraph see [0157.1.1.1] above [0158.1.6.6] In an embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 6, column 5 or 8, preferably shown in Table II A, application no. 6, column 5, or in Table II A, application no. 6, column 8, or in Table II B, application no. 6, column 8, or a homolog or a fragment thereof;
   (b) a nucleic acid molecule shown in Table I, application no. 6, column 5 or 8, preferably shown in Table I A, application no. 6, column 5, or in Table I A, application no. 6, column 8, or in Table I B, application no. 6, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 6, preferably shown in Table II A, application no. 6, column 5, or in Table II A, application no. 6, column 8, or in Table II B, application no. 6, column 8;
   (d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 6, preferably shown in Table I A, application no. 6, column 5, or in Table I A, application no. 6, column 8, or in Table I B, application no. 6, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 6;
   (i) a nucleic acid molecule encoding a polypeptide having the activity represented by a respective protein as depicted in column 5 of Table II, application no. 6;
   (j) a nucleic acid molecule comprising a polynucleotide which is obtained by amplifying a cDNA library or a genomic library using the primers in Table III, application no. 6, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto. and, optionally
3) nucleic acid molecule encoding a transit peptide, preferably encoding a plastidal transit peptide or a mitochondrial transit peptide; which are operable linked.
   (All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 6.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 6 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 6, in the same line as of the respective nucleic acid molecule according to 2) “plastidic” is depicted, or encoding a mitochondrial transit peptide, especially in case in column 6 of Table I, application no. 6, in the same line as of the respective nucleic acid molecule according to 2) “mitochondrial” is depicted.

In another embodiment thereof the expression cassette does not comprise 3) a nucleic acid molecule encoding a transit peptide, especially in case in column 6 of Table I, application no. 6, in the same line as of the respective nucleic acid molecule according to 2) “non-targeted” is depicted.

In a further embodiment, the invention relates to an expression cassette, comprising 1) a promoter, preferably a promoter selected from the group consisting of Big35S, PCUbi, Super and USP; 2) a nucleic acid molecule selected from the group consisting of
(a) a nucleic acid molecule encoding a polypeptide shown in Table II, application no. 6, column 5 or 8, preferably shown in Table II A, application no. 6, column 5, or in Table II A, application no. 6, column 8, or in Table II B, application no. 6, column 8, or a homolog or a fragment thereof;
(b) a nucleic acid molecule shown in Table I, application no. 6, column 5 or 8, preferably shown in Table I A, application no. 6, column 5, or in Table I A, application no. 6, column 8, or in Table I B, application no. 6, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 6, preferably shown in Table II A, application no. 6, column 5, or in Table II A, application no. 6, column 8, or in Table II B, application no. 6, column 8;
(d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 6, preferably shown in Table I A, application no. 6, column 5, or in Table I A, application no. 6, column 8,or in Table I B, application no. 6, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 6;
(i) a nucleic acid molecule encoding a polypeptide having the activity represented by a respective protein as depicted in column 5 of Table II, application no. 6;
(j) a nucleic acid molecule comprising a polynucleotide which is obtained by amplifying a cDNA library or a genomic library using the primers in Table III, application no. 6, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
or a nucleic acid molecule comprising a sequence which is complementary thereto; and, optionally
3) nucleic acid molecule encoding a transit peptide, preferably encoding a plastidal transit peptide or a mitochondrial transit peptide;
   which are operable linked and
   whereby, preferably, the nucleic acid molecule according to 2 (a), (b), (c), (d), (e), (f), (g), (h), (i), (j) or (k) distinguishes over the sequence depicted in Table I A and/or I B, application no. 6, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention according to 2) does not consist of the sequence shown in Table I A and/or I B, application no. 6, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 6, column 5 or 8, or the coding region thereof, respectively. In a further embodiment the nucleic acid molecule does not encode the polypeptide sequence shown in Table II A and/or II B, application no. 6, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 6, column 5 or 8 and does not encode a protein of the sequence shown in Table II A and/or II B, application no. 6, column 5 or 8.Accordingly, in one embodiment, the protein encoded by a sequence of a nucleic acid according to 2(a), (b), (c), (d), (e), (f), (g), (h), (i), (j) or (k) does not consist of the sequence shown in Table I A and/or I B, application no. 6, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 6, column 5 or 8 but less than 100%, 99.999%, 99.99%, 99.9%, 99%, 98%, 97%, 96% or 95% identical to the sequence shown in Table II A and/or II B, application no. 6, column 5 or 8.
   (All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 6.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 6 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 6, in the same line as of the respective nucleic acid molecule according to 2) “plastidic” is depicted, or encoding a mitochondrial transit peptide, especially in case in column 6 of Table I, application no. 6, in the same line as of the respective nucleic acid molecule according to 2) “mitochondrial” is depicted.

In another embodiment thereof the expression cassette does not comprise 3) a nucleic acid molecule encoding a transit peptide, especially in case in column 6 of Table I, application no. 6, in the same line as of the respective nucleic acid molecule according to 2) “non-targeted” is depicted.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 15187, or a homolog or fragment thereof, or a homolog thereof being depicted in Table I, application no. 6, column 8, in the same line as SEQ ID NO. 15187, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 15187 is depicted, plastidic is mentioned, or a mitochondrial transit peptide in case in Table I, application no. 6, in column 6 in the same line as SEQ ID NO. 15187 is depicted, mitochondrial is mentioned; which is operable linked, too.

In another embodiment thereof the expression cassette does not comprise an additional transit peptide in case in Table I, application no. 6, in column 6 in the same line as SEQ ID NO. 15187 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 15532, or a homolog or fragment thereof, or a homolog thereof being depicted in Table I, application no. 6, column 8, in the same line as SEQ ID NO. 15532, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 15532 is depicted, plastidic is mentioned, or a mitochondrial transit peptide in case in Table I, application no. 6, in column 6 in the same line as SEQ ID NO. 15532 is depicted, mitochondrial is mentioned; which is operable linked, too.

In another embodiment thereof the expression cassette does not comprise an additional transit peptide in case in Table I, application no. 6, in column 6 in the same line as SEQ ID NO. 15532 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 16263, or a homolog or fragment thereof, or a homolog thereof being depicted in Table I, application no. 6, column 8, in the same line as SEQ ID NO. 16263, preferably the coding region thereof, a homolog or a fragment thereof; which are operable linked. In an embodiment thereof the expression cassette comprises in addition c) a nucleic acid molecule encoding a transit peptide, especially a plastidal transit petide in case in Table I, application no. 6, in column 6 in the same line as SEQ ID NO. 16263 is depicted, plastidic is mentioned, or a mitochondrial transit peptide in case in Table I, application no. 6, in column 6 in the same line as SEQ ID NO. 16263 is depicted, mitochondrial is mentioned; which is operable linked, too.

In another embodiment thereof the expression cassette does not comprise an additional transit peptide in case in Table I, application no. 6, in column 6 in the same line as SEQ ID NO. 16263 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 16883, or a homolog or fragment thereof, or a homolog thereof being depicted in Table I, application no. 6, column 8, in the same line as SEQ ID NO. 16883, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 16883 is depicted, plastidic is mentioned, or a mitochondrial transit peptide in case in Table I, application no. 6, in column 6 in the same line as SEQ ID NO. 16883 is depicted, mitochondrial is mentioned; which is operable linked, too.

In another embodiment thereof the expression cassette does not comprise an additional transit peptide in case in Table I, application no. 6, in column 6 in the same line as SEQ ID NO. 16883 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17451, or a homolog or fragment thereof, or a homolog thereof being depicted in Table I, application no. 6, column 8, in the same line as SEQ ID NO. 17451, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 17451 is depicted, plastidic is mentioned, or a mitochondrial transit peptide in case in Table I, application no. 6, in column 6 in the same line as SEQ ID NO. 17451 is depicted, mitochondrial is mentioned; which is operable linked, too.

In another embodiment thereof the expression cassette does not comprise an additional transit peptide in case in Table I, application no. 6, in column 6 in the same line as SEQ ID NO. 17451 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 385, or a homolog or fragment thereof, or a homolog thereof being depicted in Table I, application no. 6, column 8, in the same line as SEQ ID NO. 385, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 385 is depicted, plastidic is mentioned, or a mitochondrial transit peptide in case in Table I, application no. 6, in column 6 in the same line as SEQ ID NO. 385 is depicted, mitochondrial is mentioned; which is operable linked, too.

In another embodiment thereof the expression cassette does not comprise an additional transit peptide in case in Table I, application no. 6, in column 6 in the same line as SEQ ID NO. 385 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17637, or a homolog or fragment thereof, or a homolog thereof being depicted in Table I, application no. 6, column 8, in the same line as SEQ ID NO. 17637, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 17637 is depicted, plastidic is mentioned, or a mitochondrial transit peptide in case in Table I, application no. 6, in column 6 in the same line as SEQ ID NO. 17637 is depicted, mitochondrial is mentioned; which is operable linked, too.

In another embodiment thereof the expression cassette does not comprise an additional transit peptide in case in Table I, application no. 6, in column 6 in the same line as SEQ ID NO. 17637 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 102086, or a homolog or fragment thereof, or a homolog thereof being depicted in Table I, application no. 6, column 8, in the same line as SEQ ID NO. 102086, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 102086 is depicted, plastidic is mentioned, or a mitochondrial transit peptide in case in Table I, application no. 6, in column 6 in the same line as SEQ ID NO. 102086 is depicted, mitochondrial is mentioned; which is operable linked, too.

In another embodiment thereof the expression cassette does not comprise an additional transit peptide in case in Table I, application no. 6, in column 6 in the same line as SEQ ID NO. 102086 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17701, or a homolog or fragment thereof, or a homolog thereof being depicted in Table I, application no. 6, column 8, in the same line as SEQ ID NO. 17701, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 17701 is depicted, plastidic is mentioned, or a mitochondrial transit peptide in case in Table I, application no. 6, in column 6 in the same line as SEQ ID NO. 17701 is depicted, mitochondrial is mentioned; which is operable linked, too.

In another embodiment thereof the expression cassette does not comprise an additional transit peptide in case in Table I, application no. 6, in column 6 in the same line as SEQ ID NO. 17701 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17968, or a homolog or fragment thereof, or a homolog thereof being depicted in Table I, application no. 6, column 8, in the same line as SEQ ID NO. 17968, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 17968 is depicted, plastidic is mentioned, or a mitochondrial transit peptide in case in Table I, application no. 6, in column 6 in the same line as SEQ ID NO. 17968 is depicted, mitochondrial is mentioned; which is operable linked, too.

In another embodiment thereof the expression cassette does not comprise an additional transit peptide in case in Table I, application no. 6, in column 6 in the same line as SEQ ID NO. 17968 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 18070, or a homolog or fragment thereof, or a homolog thereof being depicted in Table I, application no. 6, column 8, in the same line as SEQ ID NO. 18070, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 18070 is depicted, plastidic is mentioned, or a mitochondrial transit peptide in case in Table I, application no. 6, in column 6 in the same line as SEQ ID NO. 18070 is depicted, mitochondrial is mentioned; which is operable linked, too.

In another embodiment thereof the expression cassette does not comprise an additional transit peptide in case in Table I, application no. 6, in column 6 in the same line as SEQ ID NO. 18070 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 18122, or a homolog or fragment thereof, or a homolog thereof being depicted in Table I, application no. 6, column 8, in the same line as SEQ ID NO. 18122, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 18122 is depicted, plastidic is mentioned, or a mitochondrial transit peptide in case in Table I, application no. 6, in column 6 in the same line as SEQ ID NO. 18122 is depicted, mitochondrial is mentioned; which is operable linked, too.

In another embodiment thereof the expression cassette does not comprise an additional transit peptide in case in Table I, application no. 6, in column 6 in the same line as SEQ ID NO. 18122 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 18235, or a homolog or fragment thereof, or a homolog thereof being depicted in Table I, application no. 6, column 8, in the same line as SEQ ID NO. 18235, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 18235 is depicted, plastidic is mentioned, or a mitochondrial transit peptide in case in Table I, application no. 6, in column 6 in the same line as SEQ ID NO. 18235 is depicted, mitochondrial is mentioned; which is operable linked, too.

In another embodiment thereof the expression cassette does not comprise an additional transit peptide in case in Table I, application no. 6, in column 6 in the same line as SEQ ID NO. 18235 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 18869, or a homolog or fragment thereof, or a homolog thereof being depicted in Table I, application no. 6, column 8, in the same line as SEQ ID NO. 18869, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 18869 is depicted, plastidic is mentioned, or a mitochondrial transit peptide in case in Table I, application no. 6, in column 6 in the same line as SEQ ID NO. 18869 is depicted, mitochondrial is mentioned; which is operable linked, too.

In another embodiment thereof the expression cassette does not comprise an additional transit peptide in case in Table I, application no. 6, in column 6 in the same line as SEQ ID NO. 18869 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 102119, or a homolog or fragment thereof, or a homolog thereof being depicted in Table I, application no. 6, column 8, in the same line as SEQ ID NO. 102119, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 102119 is depicted, plastidic is mentioned, or a mitochondrial transit peptide in case in Table I, application no. 6, in column 6 in the same line as SEQ ID NO. 102119 is depicted, mitochondrial is mentioned; which is operable linked, too.

In another embodiment thereof the expression cassette does not comprise an additional transit peptide in case in Table I, application no. 6, in column 6 in the same line as SEQ ID NO. 102119 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 19419, or a homolog or fragment thereof, or a homolog thereof being depicted in Table I, application no. 6, column 8, in the same line as SEQ ID NO. 19419, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 19419 is depicted, plastidic is mentioned, or a mitochondrial transit peptide in case in Table I, application no. 6, in column 6 in the same line as SEQ ID NO. 19419 is depicted, mitochondrial is mentioned; which is operable linked, too.

In another embodiment thereof the expression cassette does not comprise an additional transit peptide in case in Table I, application no. 6, in column 6 in the same line as SEQ ID NO. 19419 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 19502, or a homolog or fragment thereof, or a homolog thereof being depicted in Table I, application no. 6, column 8, in the same line as SEQ ID NO. 19502, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 19502 is depicted, plastidic is mentioned, or a mitochondrial transit peptide in case in Table I, application no. 6, in column 6 in the same line as SEQ ID NO. 19502 is depicted, mitochondrial is mentioned; which is operable linked, too.

In another embodiment thereof the expression cassette does not comprise an additional transit peptide in case in Table I, application no. 6, in column 6 in the same line as SEQ ID NO. 19502 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 19874, or a homolog or fragment thereof, or a homolog thereof being depicted in Table I, application no. 6, column 8, in the same line as SEQ ID NO. 19874, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 19874 is depicted, plastidic is mentioned, or a mitochondrial transit peptide in case in Table I, application no. 6, in column 6 in the same line as SEQ ID NO. 19874 is depicted, mitochondrial is mentioned; which is operable linked, too.

In another embodiment thereof the expression cassette does not comprise an additional transit peptide in case in Table I, application no. 6, in column 6 in the same line as SEQ ID NO. 19874 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 19919, or a homolog or fragment thereof, or a homolog thereof being depicted in Table I, application no. 6, column 8, in the same line as SEQ ID NO. 19919, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 19919 is depicted, plastidic is mentioned, or a mitochondrial transit peptide in case in Table I, application no. 6, in column 6 in the same line as SEQ ID NO. 19919 is depicted, mitochondrial is mentioned; which is operable linked, too.

In another embodiment thereof the expression cassette does not comprise an additional transit peptide in case in Table I, application no. 6, in column 6 in the same line as SEQ ID NO. 19919 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 20346, or a homolog or fragment thereof, or a homolog thereof being depicted in Table I, application no. 6, column 8, in the same line as SEQ ID NO. 20346, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 20346 is depicted, plastidic is mentioned, or a mitochondrial transit peptide in case in Table I, application no. 6, in column 6 in the same line as SEQ ID NO. 20346 is depicted, mitochondrial is mentioned; which is operable linked, too.

In another embodiment thereof the expression cassette does not comprise an additional transit peptide in case in Table I, application no. 6, in column 6 in the same line as SEQ ID NO. 20346 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 21159, or a homolog or fragment thereof, or a homolog thereof being depicted in Table I, application no. 6, column 8, in the same line as SEQ ID NO. 21159, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 21159 is depicted, plastidic is mentioned, or a mitochondrial transit peptide in case in Table I, application no. 6, in column 6 in the same line as SEQ ID NO. 21159 is depicted, mitochondrial is mentioned; which is operable linked, too.

In another embodiment thereof the expression cassette does not comprise an additional transit peptide in case in Table I, application no. 6, in column 6 in the same line as SEQ ID NO. 21159 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 21497, or a homolog or fragment thereof, or a homolog thereof being depicted in Table I, application no. 6, column 8, in the same line as SEQ ID NO. 21497, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 21497 is depicted, plastidic is mentioned, or a mitochondrial transit peptide in case in Table I, application no. 6, in column 6 in the same line as SEQ ID NO. 21497 is depicted, mitochondrial is mentioned; which is operable linked, too.

In another embodiment thereof the expression cassette does not comprise an additional transit peptide in case in Table I, application no. 6, in column 6 in the same line as SEQ ID NO. 21497 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 22249, or a homolog or fragment thereof, or a homolog thereof being depicted in Table I, application no. 6, column 8, in the same line as SEQ ID NO. 22249, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 22249 is depicted, plastidic is mentioned, or a mitochondrial transit peptide in case in Table I, application no. 6, in column 6 in the same line as SEQ ID NO. 22249 is depicted, mitochondrial is mentioned; which is operable linked, too.

In another embodiment thereof the expression cassette does not comprise an additional transit peptide in case in Table I, application no. 6, in column 6 in the same line as SEQ ID NO. 22249 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 22611, or a homolog or fragment thereof, or a homolog thereof being depicted in Table I, application no. 6, column 8, in the same line as SEQ ID NO. 22611, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 22611 is depicted, plastidic is mentioned, or a mitochondrial transit peptide in case in Table I, application no. 6, in column 6 in the same line as SEQ ID NO. 22611 is depicted, mitochondrial is mentioned; which is operable linked, too.

In another embodiment thereof the expression cassette does not comprise an additional transit peptide in case in Table I, application no. 6, in column 6 in the same line as SEQ ID NO. 22611 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 22832, or a homolog or fragment thereof, or a homolog thereof being depicted in Table I, application no. 6, column 8, in the same line as SEQ ID NO. 22832, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 22832 is depicted, plastidic is mentioned, or a mitochondrial transit peptide in case in Table I, application no. 6, in column 6 in the same line as SEQ ID NO. 22832 is depicted, mitochondrial is mentioned; which is operable linked, too.

In another embodiment thereof the expression cassette does not comprise an additional transit peptide in case in Table I, application no. 6, in column 6 in the same line as SEQ ID NO. 22832 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 1815, or a homolog or fragment thereof, or a homolog thereof being depicted in Table I, application no. 6, column 8, in the same line as SEQ ID NO. 1815, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 1815 is depicted, plastidic is mentioned, or a mitochondrial transit peptide in case in Table I, application no. 6, in column 6 in the same line as SEQ ID NO. 1815 is depicted, mitochondrial is mentioned; which is operable linked, too.

In another embodiment thereof the expression cassette does not comprise an additional transit peptide in case in Table I, application no. 6, in column 6 in the same line as SEQ ID NO. 1815 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68727, or a homolog or fragment thereof, or a homolog thereof being depicted in Table I, application no. 6, column 8, in the same line as SEQ ID NO. 68727, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 68727 is depicted, plastidic is mentioned, or a mitochondrial transit peptide in case in Table I, application no. 6, in column 6 in the same line as SEQ ID NO. 68727 is depicted, mitochondrial is mentioned; which is operable linked, too.

In another embodiment thereof the expression cassette does not comprise an additional transit peptide in case in Table I, application no. 6, in column 6 in the same line as SEQ ID NO. 68727 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68777, or a homolog or fragment thereof, or a homolog thereof being depicted in Table I, application no. 6, column 8, in the same line as SEQ ID NO. 68777, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 68777 is depicted, plastidic is mentioned, or a mitochondrial transit peptide in case in Table I, application no. 6, in column 6 in the same line as SEQ ID NO. 68777 is depicted, mitochondrial is mentioned; which is operable linked, too.

In another embodiment thereof the expression cassette does not comprise an additional transit peptide in case in Table I, application no. 6, in column 6 in the same line as SEQ ID NO. 68777 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 2367, or a homolog or fragment thereof, or a homolog thereof being depicted in Table I, application no. 6, column 8, in the same line as SEQ ID NO. 2367, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 2367 is depicted, plastidic is mentioned, or a mitochondrial transit peptide in case in Table I, application no. 6, in column 6 in the same line as SEQ ID NO. 2367 is depicted, mitochondrial is mentioned; which is operable linked, too.

In another embodiment thereof the expression cassette does not comprise an additional transit peptide in case in Table I, application no. 6, in column 6 in the same line as SEQ ID NO. 2367 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 2935, or a homolog or fragment thereof, or a homolog thereof being depicted in Table I, application no. 6, column 8, in the same line as SEQ ID NO. 2935, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 2935 is depicted, plastidic is mentioned, or a mitochondrial transit peptide in case in Table I, application no. 6, in column 6 in the same line as SEQ ID NO. 2935 is depicted, mitochondrial is mentioned; which is operable linked, too.

In another embodiment thereof the expression cassette does not comprise an additional transit peptide in case in Table I, application no. 6, in column 6 in the same line as SEQ ID NO. 2935 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 24232, or a homolog or fragment thereof, or a homolog thereof being depicted in Table I, application no. 6, column 8, in the same line as SEQ ID NO. 24232, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 24232 is depicted, plastidic is mentioned, or a mitochondrial transit peptide in case in Table I, application no. 6, in column 6 in the same line as SEQ ID NO. 24232 is depicted, mitochondrial is mentioned; which is operable linked, too.

In another embodiment thereof the expression cassette does not comprise an additional transit peptide in case in Table I, application no. 6, in column 6 in the same line as SEQ ID NO. 24232 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68849, or a homolog or fragment thereof, or a homolog thereof being depicted in Table I, application no. 6, column 8, in the same line as SEQ ID NO. 68849, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 68849 is depicted, plastidic is mentioned, or a mitochondrial transit peptide in case in Table I, application no. 6, in column 6 in the same line as SEQ ID NO. 68849 is depicted, mitochondrial is mentioned; which is operable linked, too.

In another embodiment thereof the expression cassette does not comprise an additional transit peptide in case in Table I, application no. 6, in column 6 in the same line as SEQ ID NO. 68849 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4040, or a homolog or fragment thereof, or a homolog thereof being depicted in Table I, application no. 6, column 8, in the same line as SEQ ID NO. 4040, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 4040 is depicted, plastidic is mentioned, or a mitochondrial transit peptide in case in Table I, application no. 6, in column 6 in the same line as SEQ ID NO. 4040 is depicted, mitochondrial is mentioned; which is operable linked, too.

In another embodiment thereof the expression cassette does not comprise an additional transit peptide in case in Table I, application no. 6, in column 6 in the same line as SEQ ID NO. 4040 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 24311, or a homolog or fragment thereof, or a homolog thereof being depicted in Table I, application no. 6, column 8, in the same line as SEQ ID NO. 24311, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 24311 is depicted, plastidic is mentioned, or a mitochondrial transit peptide in case in Table I, application no. 6, in column 6 in the same line as SEQ ID NO. 24311 is depicted, mitochondrial is mentioned; which is operable linked, too.

In another embodiment thereof the expression cassette does not comprise an additional transit peptide in case in Table I, application no. 6, in column 6 in the same line as SEQ ID NO. 24311 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4348, or a homolog or fragment thereof, or a homolog thereof being depicted in Table I, application no. 6, column 8, in the same line as SEQ ID NO. 4348, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 4348 is depicted, plastidic is mentioned, or a mitochondrial transit peptide in case in Table I, application no. 6, in column 6 in the same line as SEQ ID NO. 4348 is depicted, mitochondrial is mentioned; which is operable linked, too.

In another embodiment thereof the expression cassette does not comprise an additional transit peptide in case in Table I, application no. 6, in column 6 in the same line as SEQ ID NO. 4348 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68987, or a homolog or fragment thereof, or a homolog thereof being depicted in Table I, application no. 6, column 8, in the same line as SEQ ID NO. 68987, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 68987 is depicted, plastidic is mentioned, or a mitochondrial transit peptide in case in Table I, application no. 6, in column 6 in the same line as SEQ ID NO. 68987 is depicted, mitochondrial is mentioned; which is operable linked, too.

In another embodiment thereof the expression cassette does not comprise an additional transit peptide in case in Table I, application no. 6, in column 6 in the same line as SEQ ID NO. 68987 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 25283, or a homolog or fragment thereof, or a homolog thereof being depicted in Table I, application no. 6, column 8, in the same line as SEQ ID NO. 25283, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 25283 is depicted, plastidic is mentioned, or a mitochondrial transit peptide in case in Table I, application no. 6, in column 6 in the same line as SEQ ID NO. 25283 is depicted, mitochondrial is mentioned; which is operable linked, too.

In another embodiment thereof the expression cassette does not comprise an additional transit peptide in case in Table I, application no. 6, in column 6 in the same line as SEQ ID NO. 25283 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4904, or a homolog or fragment thereof, or a homolog thereof being depicted in Table I, application no. 6, column 8, in the same line as SEQ ID NO. 4904, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 4904 is depicted, plastidic is mentioned, or a mitochondrial transit peptide in case in Table I, application no. 6, in column 6 in the same line as SEQ ID NO. 4904 is depicted, mitochondrial is mentioned; which is operable linked, too.

In another embodiment thereof the expression cassette does not comprise an additional transit peptide in case in Table I, application no. 6, in column 6 in the same line as SEQ ID NO. 4904 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 69574, or a homolog or fragment thereof, or a homolog thereof being depicted in Table I, application no. 6, column 8, in the same line as SEQ ID NO. 69574, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 69574 is depicted, plastidic is mentioned, or a mitochondrial transit peptide in case in Table I, application no. 6, in column 6 in the same line as SEQ ID NO. 69574 is depicted, mitochondrial is mentioned; which is operable linked, too.

In another embodiment thereof the expression cassette does not comprise an additional transit peptide in case in Table I, application no. 6, in column 6 in the same line as SEQ ID NO. 69574 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 102223, or a homolog or fragment thereof, or a homolog thereof being depicted in Table I, application no. 6, column 8, in the same line as SEQ ID NO. 102223, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 102223 is depicted, plastidic is mentioned, or a mitochondrial transit peptide in case in Table I, application no. 6, in column 6 in the same line as SEQ ID NO. 102223 is depicted, mitochondrial is mentioned; which is operable linked, too.

In another embodiment thereof the expression cassette does not comprise an additional transit peptide in case in Table I, application no. 6, in column 6 in the same line as SEQ ID NO. 102223 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 102373, or a homolog or fragment thereof, or a homolog thereof being depicted in Table I, application no. 6, column 8, in the same line as SEQ ID NO. 102373, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 102373 is depicted, plastidic is mentioned, or a mitochondrial transit peptide in case in Table I, application no. 6, in column 6 in the same line as SEQ ID NO. 102373 is depicted, mitochondrial is mentioned; which is operable linked, too.

In another embodiment thereof the expression cassette does not comprise an additional transit peptide in case in Table I, application no. 6, in column 6 in the same line as SEQ ID NO. 102373 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 70006, or a homolog or fragment thereof, or a homolog thereof being depicted in Table I, application no. 6, column 8, in the same line as SEQ ID NO. 70006, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 70006 is depicted, plastidic is mentioned, or a mitochondrial transit peptide in case in Table I, application no. 6, in column 6 in the same line as SEQ ID NO. 70006 is depicted, mitochondrial is mentioned; which is operable linked, too.

In another embodiment thereof the expression cassette does not comprise an additional transit peptide in case in Table I, application no. 6, in column 6 in the same line as SEQ ID NO. 70006 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 102899, or a homolog or fragment thereof, or a homolog thereof being depicted in Table I, application no. 6, column 8, in the same line as SEQ ID NO. 102899, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 102899 is depicted, plastidic is mentioned, or a mitochondrial transit peptide in case in Table I, application no. 6, in column 6 in the same line as SEQ ID NO. 102899 is depicted, mitochondrial is mentioned; which is operable linked, too.

In another embodiment thereof the expression cassette does not comprise an additional transit peptide in case in Table I, application no. 6, in column 6 in the same line as SEQ ID NO. 102899 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 70038, or a homolog or fragment thereof, or a homolog thereof being depicted in Table I, application no. 6, column 8, in the same line as SEQ ID NO. 70038, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 70038 is depicted, plastidic is mentioned, or a mitochondrial transit peptide in case in Table I, application no. 6, in column 6 in the same line as SEQ ID NO. 70038 is depicted, mitochondrial is mentioned; which is operable linked, too.

In another embodiment thereof the expression cassette does not comprise an additional transit peptide in case in Table I, application no. 6, in column 6 in the same line as SEQ ID NO. 70038 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 5493, or a homolog or fragment thereof, or a homolog thereof being depicted in Table I, application no. 6, column 8, in the same line as SEQ ID NO. 5493, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 5493 is depicted, plastidic is mentioned, or a mitochondrial transit peptide in case in Table I, application no. 6, in column 6 in the same line as SEQ ID NO. 5493 is depicted, mitochondrial is mentioned; which is operable linked, too.

In another embodiment thereof the expression cassette does not comprise an additional transit peptide in case in Table I, application no. 6, in column 6 in the same line as SEQ ID NO. 5493 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 5557, or a homolog or fragment thereof, or a homolog thereof being depicted in Table I, application no. 6, column 8, in the same line as SEQ ID NO. 5557, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 5557 is depicted, plastidic is mentioned, or a mitochondrial transit peptide in case in Table I, application no. 6, in column 6 in the same line as SEQ ID NO. 5557 is depicted, mitochondrial is mentioned; which is operable linked, too.

In another embodiment thereof the expression cassette does not comprise an additional transit peptide in case in Table I, application no. 6, in column 6 in the same line as SEQ ID NO. 5557 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 26434, or a homolog or fragment thereof, or a homolog thereof being depicted in Table I, application no. 6, column 8, in the same line as SEQ ID NO. 26434, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 26434 is depicted, plastidic is mentioned, or a mitochondrial transit peptide in case in Table I, application no. 6, in column 6 in the same line as SEQ ID NO. 26434 is depicted, mitochondrial is mentioned; which is operable linked, too.

In another embodiment thereof the expression cassette does not comprise an additional transit peptide in case in Table I, application no. 6, in column 6 in the same line as SEQ ID NO. 26434 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6040, or a homolog or fragment thereof, or a homolog thereof being depicted in Table I, application no. 6, column 8, in the same line as SEQ ID NO. 6040, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 6040 is depicted, plastidic is mentioned, or a mitochondrial transit peptide in case in Table I, application no. 6, in column 6 in the same line as SEQ ID NO. 6040 is depicted, mitochondrial is mentioned; which is operable linked, too.

In another embodiment thereof the expression cassette does not comprise an additional transit peptide in case in Table I, application no. 6, in column 6 in the same line as SEQ ID NO. 6040 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 27021, or a homolog or fragment thereof, or a homolog thereof being depicted in Table I, application no. 6, column 8, in the same line as SEQ ID NO. 27021, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 27021 is depicted, plastidic is mentioned, or a mitochondrial transit peptide in case in Table I, application no. 6, in column 6 in the same line as SEQ ID NO. 27021 is depicted, mitochondrial is mentioned; which is operable linked, too.

In another embodiment thereof the expression cassette does not comprise an additional transit peptide in case in Table I, application no. 6, in column 6 in the same line as SEQ ID NO. 27021 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 71763, or a homolog or fragment thereof, or a homolog thereof being depicted in Table I, application no. 6, column 8, in the same line as SEQ ID NO. 71763, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 71763 is depicted, plastidic is mentioned, or a mitochondrial transit peptide in case in Table I, application no. 6, in column 6 in the same line as SEQ ID NO. 71763 is depicted, mitochondrial is mentioned; which is operable linked, too.

In another embodiment thereof the expression cassette does not comprise an additional transit peptide in case in Table I, application no. 6, in column 6 in the same line as SEQ ID NO. 71763 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 27882, or a homolog or fragment thereof, or a homolog thereof being depicted in Table I, application no. 6, column 8, in the same line as SEQ ID NO. 27882, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 27882 is depicted, plastidic is mentioned, or a mitochondrial transit peptide in case in Table I, application no. 6, in column 6 in the same line as SEQ ID NO. 27882 is depicted, mitochondrial is mentioned; which is operable linked, too.

In another embodiment thereof the expression cassette does not comprise an additional transit peptide in case in Table I, application no. 6, in column 6 in the same line as SEQ ID NO. 27882 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 28738, or a homolog or fragment thereof, or a homolog thereof being depicted in Table I, application no. 6, column 8, in the same line as SEQ ID NO. 28738, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 28738 is depicted, plastidic is mentioned, or a mitochondrial transit peptide in case in Table I, application no. 6, in column 6 in the same line as SEQ ID NO. 28738 is depicted, mitochondrial is mentioned; which is operable linked, too.

In another embodiment thereof the expression cassette does not comprise an additional transit peptide in case in Table I, application no. 6, in column 6 in the same line as SEQ ID NO. 28738 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 29286, or a homolog or fragment thereof, or a homolog thereof being depicted in Table I, application no. 6, column 8, in the same line as SEQ ID NO. 29286, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 29286 is depicted, plastidic is mentioned, or a mitochondrial transit peptide in case in Table I, application no. 6, in column 6 in the same line as SEQ ID NO. 29286 is depicted, mitochondrial is mentioned; which is operable linked, too.

In another embodiment thereof the expression cassette does not comprise an additional transit peptide in case in Table I, application no. 6, in column 6 in the same line as SEQ ID NO. 29286 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 29397, or a homolog or fragment thereof, or a homolog thereof being depicted in Table I, application no. 6, column 8, in the same line as SEQ ID NO. 29397, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 29397 is depicted, plastidic is mentioned, or a mitochondrial transit peptide in case in Table I, application no. 6, in column 6 in the same line as SEQ ID NO. 29397 is depicted, mitochondrial is mentioned; which is operable linked, too.

In another embodiment thereof the expression cassette does not comprise an additional transit peptide in case in Table I, application no. 6, in column 6 in the same line as SEQ ID NO. 29397 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 29500, or a homolog or fragment thereof, or a homolog thereof being depicted in Table I, application no. 6, column 8, in the same line as SEQ ID NO. 29500, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 29500 is depicted, plastidic is mentioned, or a mitochondrial transit peptide in case in Table I, application no. 6, in column 6 in the same line as SEQ ID NO. 29500 is depicted, mitochondrial is mentioned; which is operable linked, too.

In another embodiment thereof the expression cassette does not comprise an additional transit peptide in case in Table I, application no. 6, in column 6 in the same line as SEQ ID NO. 29500 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 72520, or a homolog or fragment thereof, or a homolog thereof being depicted in Table I, application no. 6, column 8, in the same line as SEQ ID NO. 72520, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 72520 is depicted, plastidic is mentioned, or a mitochondrial transit peptide in case in Table I, application no. 6, in column 6 in the same line as SEQ ID NO. 72520 is depicted, mitochondrial is mentioned; which is operable linked, too.

In another embodiment thereof the expression cassette does not comprise an additional transit peptide in case in Table I, application no. 6, in column 6 in the same line as SEQ ID NO. 72520 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 31717, or a homolog or fragment thereof, or a homolog thereof being depicted in Table I, application no. 6, column 8, in the same line as SEQ ID NO. 31717, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 31717 is depicted, plastidic is mentioned, or a mitochondrial transit peptide in case in Table I, application no. 6, in column 6 in the same line as SEQ ID NO. 31717 is depicted, mitochondrial is mentioned; which is operable linked, too.

In another embodiment thereof the expression cassette does not comprise an additional transit peptide in case in Table I, application no. 6, in column 6 in the same line as SEQ ID NO. 31717 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 31926, or a homolog or fragment thereof, or a homolog thereof being depicted in Table I, application no. 6, column 8, in the same line as SEQ ID NO. 31926, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 31926 is depicted, plastidic is mentioned, or a mitochondrial transit peptide in case in Table I, application no. 6, in column 6 in the same line as SEQ ID NO. 31926 is depicted, mitochondrial is mentioned; which is operable linked, too.

In another embodiment thereof the expression cassette does not comprise an additional transit peptide in case in Table I, application no. 6, in column 6 in the same line as SEQ ID NO. 31926 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 73719, or a homolog or fragment thereof, or a homolog thereof being depicted in Table I, application no. 6, column 8, in the same line as SEQ ID NO. 73719, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 73719 is depicted, plastidic is mentioned, or a mitochondrial transit peptide in case in Table I, application no. 6, in column 6 in the same line as SEQ ID NO. 73719 is depicted, mitochondrial is mentioned; which is operable linked, too.

In another embodiment thereof the expression cassette does not comprise an additional transit peptide in case in Table I, application no. 6, in column 6 in the same line as SEQ ID NO. 73719 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 74163, or a homolog or fragment thereof, or a homolog thereof being depicted in Table I, application no. 6, column 8, in the same line as SEQ ID NO. 74163, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 74163 is depicted, plastidic is mentioned, or a mitochondrial transit peptide in case in Table I, application no. 6, in column 6 in the same line as SEQ ID NO. 74163 is depicted, mitochondrial is mentioned; which is operable linked, too.

In another embodiment thereof the expression cassette does not comprise an additional transit peptide in case in Table I, application no. 6, in column 6 in the same line as SEQ ID NO. 74163 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 102941, or a homolog or fragment thereof, or a homolog thereof being depicted in Table I, application no. 6, column 8, in the same line as SEQ ID NO. 102941, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 102941 is depicted, plastidic is mentioned, or a mitochondrial transit peptide in case in Table I, application no. 6, in column 6 in the same line as SEQ ID NO. 102941 is depicted, mitochondrial is mentioned; which is operable linked, too.

In another embodiment thereof the expression cassette does not comprise an additional transit peptide in case in Table I, application no. 6, in column 6 in the same line as SEQ ID NO. 102941 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6510, or a homolog or fragment thereof, or a homolog thereof being depicted in Table I, application no. 6, column 8, in the same line as SEQ ID NO. 6510, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 6510 is depicted, plastidic is mentioned, or a mitochondrial transit peptide in case in Table I, application no. 6, in column 6 in the same line as SEQ ID NO. 6510 is depicted, mitochondrial is mentioned; which is operable linked, too.

In another embodiment thereof the expression cassette does not comprise an additional transit peptide in case in Table I, application no. 6, in column 6 in the same line as SEQ ID NO. 6510 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 32308, or a homolog or fragment thereof, or a homolog thereof being depicted in Table I, application no. 6, column 8, in the same line as SEQ ID NO. 32308, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 32308 is depicted, plastidic is mentioned, or a mitochondrial transit peptide in case in Table I, application no. 6, in column 6 in the same line as SEQ ID NO. 32308 is depicted, mitochondrial is mentioned; which is operable linked, too.

In another embodiment thereof the expression cassette does not comprise an additional transit peptide in case in Table I, application no. 6, in column 6 in the same line as SEQ ID NO. 32308 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 74662, or a homolog or fragment thereof, or a homolog thereof being depicted in Table I, application no. 6, column 8, in the same line as SEQ ID NO. 74662, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 74662 is depicted, plastidic is mentioned, or a mitochondrial transit peptide in case in Table I, application no. 6, in column 6 in the same line as SEQ ID NO. 74662 is depicted, mitochondrial is mentioned; which is operable linked, too.

In another embodiment thereof the expression cassette does not comprise an additional transit peptide in case in Table I, application no. 6, in column 6 in the same line as SEQ ID NO. 74662 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 33596, or a homolog or fragment thereof, or a homolog thereof being depicted in Table I, application no. 6, column 8, in the same line as SEQ ID NO. 33596, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 33596 is depicted, plastidic is mentioned, or a mitochondrial transit peptide in case in Table I, application no. 6, in column 6 in the same line as SEQ ID NO. 33596 is depicted, mitochondrial is mentioned; which is operable linked, too.

In another embodiment thereof the expression cassette does not comprise an additional transit peptide in case in Table I, application no. 6, in column 6 in the same line as SEQ ID NO. 33596 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 34044, or a homolog or fragment thereof, or a homolog thereof being depicted in Table I, application no. 6, column 8, in the same line as SEQ ID NO. 34044, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 34044 is depicted, plastidic is mentioned, or a mitochondrial transit peptide in case in Table I, application no. 6, in column 6 in the same line as SEQ ID NO. 34044 is depicted, mitochondrial is mentioned; which is operable linked, too.

In another embodiment thereof the expression cassette does not comprise an additional transit peptide in case in Table I, application no. 6, in column 6 in the same line as SEQ ID NO. 34044 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 103433, or a homolog or fragment thereof, or a homolog thereof being depicted in Table I, application no. 6, column 8, in the same line as SEQ ID NO. 103433, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 103433 is depicted, plastidic is mentioned, or a mitochondrial transit peptide in case in Table I, application no. 6, in column 6 in the same line as SEQ ID NO. 103433 is depicted, mitochondrial is mentioned; which is operable linked, too.

In another embodiment thereof the expression cassette does not comprise an additional transit peptide in case in Table I, application no. 6, in column 6 in the same line as SEQ ID NO. 103433 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 34889, or a homolog or fragment thereof, or a homolog thereof being depicted in Table I, application no. 6, column 8, in the same line as SEQ ID NO. 34889, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 34889 is depicted, plastidic is mentioned, or a mitochondrial transit peptide in case in Table I, application no. 6, in column 6 in the same line as SEQ ID NO. 34889 is depicted, mitochondrial is mentioned; which is operable linked, too.

In another embodiment thereof the expression cassette does not comprise an additional transit peptide in case in Table I, application no. 6, in column 6 in the same line as SEQ ID NO. 34889 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 103958, or a homolog or fragment thereof, or a homolog thereof being depicted in Table I, application no. 6, column 8, in the same line as SEQ ID NO. 103958, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 103958 is depicted, plastidic is mentioned, or a mitochondrial transit peptide in case in Table I, application no. 6, in column 6 in the same line as SEQ ID NO. 103958 is depicted, mitochondrial is mentioned; which is operable linked, too.

In another embodiment thereof the expression cassette does not comprise an additional transit peptide in case in Table I, application no. 6, in column 6 in the same line as SEQ ID NO. 103958 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 6818, or a homolog or fragment thereof, or a homolog thereof being depicted in Table I, application no. 6, column 8, in the same line as SEQ ID NO. 6818, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 6818 is depicted, plastidic is mentioned, or a mitochondrial transit peptide in case in Table I, application no. 6, in column 6 in the same line as SEQ ID NO. 6818 is depicted, mitochondrial is mentioned; which is operable linked, too.

In another embodiment thereof the expression cassette does not comprise an additional transit peptide in case in Table I, application no. 6, in column 6 in the same line as SEQ ID NO. 6818 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 35733, or a homolog or fragment thereof, or a homolog thereof being depicted in Table I, application no. 6, column 8, in the same line as SEQ ID NO. 35733, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 35733 is depicted, plastidic is mentioned, or a mitochondrial transit peptide in case in Table I, application no. 6, in column 6 in the same line as SEQ ID NO. 35733 is depicted, mitochondrial is mentioned; which is operable linked, too.

In another embodiment thereof the expression cassette does not comprise an additional transit peptide in case in Table I, application no. 6, in column 6 in the same line as SEQ ID NO. 35733 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 7269, or a homolog or fragment thereof, or a homolog thereof being depicted in Table I, application no. 6, column 8, in the same line as SEQ ID NO. 7269, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 7269 is depicted, plastidic is mentioned, or a mitochondrial transit peptide in case in Table I, application no. 6, in column 6 in the same line as SEQ ID NO. 7269 is depicted, mitochondrial is mentioned; which is operable linked, too.

In another embodiment thereof the expression cassette does not comprise an additional transit peptide in case in Table I, application no. 6, in column 6 in the same line as SEQ ID NO. 7269 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7333, or a homolog or fragment thereof, or a homolog thereof being depicted in Table I, application no. 6, column 8, in the same line as SEQ ID NO. 7333, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 7333 is depicted, plastidic is mentioned, or a mitochondrial transit peptide in case in Table I, application no. 6, in column 6 in the same line as SEQ ID NO. 7333 is depicted, mitochondrial is mentioned; which is operable linked, too.

In another embodiment thereof the expression cassette does not comprise an additional transit peptide in case in Table I, application no. 6, in column 6 in the same line as SEQ ID NO. 7333 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7686, or a homolog or fragment thereof, or a homolog thereof being depicted in Table I, application no. 6, column 8, in the same line as SEQ ID NO. 7686, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 7686 is depicted, plastidic is mentioned, or a mitochondrial transit peptide in case in Table I, application no. 6, in column 6 in the same line as SEQ ID NO. 7686 is depicted, mitochondrial is mentioned; which is operable linked, too.

In another embodiment thereof the expression cassette does not comprise an additional transit peptide in case in Table I, application no. 6, in column 6 in the same line as SEQ ID NO. 7686 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 35967, or a homolog or fragment thereof, or a homolog thereof being depicted in Table I, application no. 6, column 8, in the same line as SEQ ID NO. 35967, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 35967 is depicted, plastidic is mentioned, or a mitochondrial transit peptide in case in Table I, application no. 6, in column 6 in the same line as SEQ ID NO. 35967 is depicted, mitochondrial is mentioned; which is operable linked, too.

In another embodiment thereof the expression cassette does not comprise an additional transit peptide in case in Table I, application no. 6, in column 6 in the same line as SEQ ID NO. 35967 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 36114, or a homolog or fragment thereof, or a homolog thereof being depicted in Table I, application no. 6, column 8, in the same line as SEQ ID NO. 36114, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 36114 is depicted, plastidic is mentioned, or a mitochondrial transit peptide in case in Table I, application no. 6, in column 6 in the same line as SEQ ID NO. 36114 is depicted, mitochondrial is mentioned; which is operable linked, too.

In another embodiment thereof the expression cassette does not comprise an additional transit peptide in case in Table I, application no. 6, in column 6 in the same line as SEQ ID NO. 36114 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7917, or a homolog or fragment thereof, or a homolog thereof being depicted in Table I, application no. 6, column 8, in the same line as SEQ ID NO. 7917, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 7917 is depicted, plastidic is mentioned, or a mitochondrial transit peptide in case in Table I, application no. 6, in column 6 in the same line as SEQ ID NO. 7917 is depicted, mitochondrial is mentioned; which is operable linked, too.

In another embodiment thereof the expression cassette does not comprise an additional transit peptide in case in Table I, application no. 6, in column 6 in the same line as SEQ ID NO. 7917 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 36623, or a homolog or fragment thereof, or a homolog thereof being depicted in Table I, application no. 6, column 8, in the same line as SEQ ID NO. 36623, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 36623 is depicted, plastidic is mentioned, or a mitochondrial transit peptide in case in Table I, application no. 6, in column 6 in the same line as SEQ ID NO. 36623 is depicted, mitochondrial is mentioned; which is operable linked, too.

In another embodiment thereof the expression cassette does not comprise an additional transit peptide in case in Table I, application no. 6, in column 6 in the same line as SEQ ID NO. 36623 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 36670, or a homolog or fragment thereof, or a homolog thereof being depicted in Table I, application no. 6, column 8, in the same line as SEQ ID NO. 36670, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 36670 is depicted, plastidic is mentioned, or a mitochondrial transit peptide in case in Table I, application no. 6, in column 6 in the same line as SEQ ID NO. 36670 is depicted, mitochondrial is mentioned; which is operable linked, too.

In another embodiment thereof the expression cassette does not comprise an additional transit peptide in case in Table I, application no. 6, in column 6 in the same line as SEQ ID NO. 36670 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 36809, or a homolog or fragment thereof, or a homolog thereof being depicted in Table I, application no. 6, column 8, in the same line as SEQ ID NO. 36809, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 36809 is depicted, plastidic is mentioned, or a mitochondrial transit peptide in case in Table I, application no. 6, in column 6 in the same line as SEQ ID NO. 36809 is depicted, mitochondrial is mentioned; which is operable linked, too.

In another embodiment thereof the expression cassette does not comprise an additional transit peptide in case in Table I, application no. 6, in column 6 in the same line as SEQ ID NO. 36809 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7941, or a homolog or fragment thereof, or a homolog thereof being depicted in Table I, application no. 6, column 8, in the same line as SEQ ID NO. 7941, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 7941 is depicted, plastidic is mentioned, or a mitochondrial transit peptide in case in Table I, application no. 6, in column 6 in the same line as SEQ ID NO. 7941 is depicted, mitochondrial is mentioned; which is operable linked, too.

In another embodiment thereof the expression cassette does not comprise an additional transit peptide in case in Table I, application no. 6, in column 6 in the same line as SEQ ID NO. 7941 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 37394, or a homolog or fragment thereof, or a homolog thereof being depicted in Table I, application no. 6, column 8, in the same line as SEQ ID NO. 37394, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 37394 is depicted, plastidic is mentioned, or a mitochondrial transit peptide in case in Table I, application no. 6, in column 6 in the same line as SEQ ID NO. 37394 is depicted, mitochondrial is mentioned; which is operable linked, too.

In another embodiment thereof the expression cassette does not comprise an additional transit peptide in case in Table I, application no. 6, in column 6 in the same line as SEQ ID NO. 37394 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 104134, or a homolog or fragment thereof, or a homolog thereof being depicted in Table I, application no. 6, column 8, in the same line as SEQ ID NO. 104134, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 104134 is depicted, plastidic is mentioned, or a mitochondrial transit peptide in case in Table I, application no. 6, in column 6 in the same line as SEQ ID NO. 104134 is depicted, mitochondrial is mentioned; which is operable linked, too.

In another embodiment thereof the expression cassette does not comprise an additional transit peptide in case in Table I, application no. 6, in column 6 in the same line as SEQ ID NO. 104134 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 37483, or a homolog or fragment thereof, or a homolog thereof being depicted in Table I, application no. 6, column 8, in the same line as SEQ ID NO. 37483, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 37483 is depicted, plastidic is mentioned, or a mitochondrial transit peptide in case in Table I, application no. 6, in column 6 in the same line as SEQ ID NO. 37483 is depicted, mitochondrial is mentioned; which is operable linked, too.

In another embodiment thereof the expression cassette does not comprise an additional transit peptide in case in Table I, application no. 6, in column 6 in the same line as SEQ ID NO. 37483 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 37539, or a homolog or fragment thereof, or a homolog thereof being depicted in Table I, application no. 6, column 8, in the same line as SEQ ID NO. 37539, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 37539 is depicted, plastidic is mentioned, or a mitochondrial transit peptide in case in Table I, application no. 6, in column 6 in the same line as SEQ ID NO. 37539 is depicted, mitochondrial is mentioned; which is operable linked, too.

In another embodiment thereof the expression cassette does not comprise an additional transit peptide in case in Table I, application no. 6, in column 6 in the same line as SEQ ID NO. 37539 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 37658, or a homolog or fragment thereof, or a homolog thereof being depicted in Table I, application no. 6, column 8, in the same line as SEQ ID NO. 37658, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 37658 is depicted, plastidic is mentioned, or a mitochondrial transit peptide in case in Table I, application no. 6, in column 6 in the same line as SEQ ID NO. 37658 is depicted, mitochondrial is mentioned; which is operable linked, too.

In another embodiment thereof the expression cassette does not comprise an additional transit peptide in case in Table I, application no. 6, in column 6 in the same line as SEQ ID NO. 37658 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 37807, or a homolog or fragment thereof, or a homolog thereof being depicted in Table I, application no. 6, column 8, in the same line as SEQ ID NO. 37807, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 37807 is depicted, plastidic is mentioned, or a mitochondrial transit peptide in case in Table I, application no. 6, in column 6 in the same line as SEQ ID NO. 37807 is depicted, mitochondrial is mentioned; which is operable linked, too.

In another embodiment thereof the expression cassette does not comprise an additional transit peptide in case in Table I, application no. 6, in column 6 in the same line as SEQ ID NO. 37807 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38289, or a homolog or fragment thereof, or a homolog thereof being depicted in Table I, application no. 6, column 8, in the same line as SEQ ID NO. 38289, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 38289 is depicted, plastidic is mentioned, or a mitochondrial transit peptide in case in Table I, application no. 6, in column 6 in the same line as SEQ ID NO. 38289 is depicted, mitochondrial is mentioned; which is operable linked, too.

In another embodiment thereof the expression cassette does not comprise an additional transit peptide in case in Table I, application no. 6, in column 6 in the same line as SEQ ID NO. 38289 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 77774, or a homolog or fragment thereof, or a homolog thereof being depicted in Table I, application no. 6, column 8, in the same line as SEQ ID NO. 77774, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 77774 is depicted, plastidic is mentioned, or a mitochondrial transit peptide in case in Table I, application no. 6, in column 6 in the same line as SEQ ID NO. 77774 is depicted, mitochondrial is mentioned; which is operable linked, too.

In another embodiment thereof the expression cassette does not comprise an additional transit peptide in case in Table I, application no. 6, in column 6 in the same line as SEQ ID NO. 77774 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7947, or a homolog or fragment thereof, or a homolog thereof being depicted in Table I, application no. 6, column 8, in the same line as SEQ ID NO. 7947, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 7947 is depicted, plastidic is mentioned, or a mitochondrial transit peptide in case in Table I, application no. 6, in column 6 in the same line as SEQ ID NO. 7947 is depicted, mitochondrial is mentioned; which is operable linked, too.

In another embodiment thereof the expression cassette does not comprise an additional transit peptide in case in Table I, application no. 6, in column 6 in the same line as SEQ ID NO. 7947 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38300, or a homolog or fragment thereof, or a homolog thereof being depicted in Table I, application no. 6, column 8, in the same line as SEQ ID NO. 38300, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 38300 is depicted, plastidic is mentioned, or a mitochondrial transit peptide in case in Table I, application no. 6, in column 6 in the same line as SEQ ID NO. 38300 is depicted, mitochondrial is mentioned; which is operable linked, too.

In another embodiment thereof the expression cassette does not comprise an additional transit peptide in case in Table I, application no. 6, in column 6 in the same line as SEQ ID NO. 38300 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7992, or a homolog or fragment thereof, or a homolog thereof being depicted in Table I, application no. 6, column 8, in the same line as SEQ ID NO. 7992, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 7992 is depicted, plastidic is mentioned, or a mitochondrial transit peptide in case in Table I, application no. 6, in column 6 in the same line as SEQ ID NO. 7992 is depicted, mitochondrial is mentioned; which is operable linked, too.

In another embodiment thereof the expression cassette does not comprise an additional transit peptide in case in Table I, application no. 6, in column 6 in the same line as SEQ ID NO. 7992 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78753, or a homolog or fragment thereof, or a homolog thereof being depicted in Table I, application no. 6, column 8, in the same line as SEQ ID NO. 78753, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 78753 is depicted, plastidic is mentioned, or a mitochondrial transit peptide in case in Table I, application no. 6, in column 6 in the same line as SEQ ID NO. 78753 is depicted, mitochondrial is mentioned; which is operable linked, too.

In another embodiment thereof the expression cassette does not comprise an additional transit peptide in case in Table I, application no. 6, in column 6 in the same line as SEQ ID NO. 78753 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78771, or a homolog or fragment thereof, or a homolog thereof being depicted in Table I, application no. 6, column 8, in the same line as SEQ ID NO. 78771, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 78771 is depicted, plastidic is mentioned, or a mitochondrial transit peptide in case in Table I, application no. 6, in column 6 in the same line as SEQ ID NO. 78771 is depicted, mitochondrial is mentioned; which is operable linked, too.

In another embodiment thereof the expression cassette does not comprise an additional transit peptide in case in Table I, application no. 6, in column 6 in the same line as SEQ ID NO. 78771 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 8363, or a homolog or fragment thereof, or a homolog thereof being depicted in Table I, application no. 6, column 8, in the same line as SEQ ID NO. 8363, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 8363 is depicted, plastidic is mentioned, or a mitochondrial transit peptide in case in Table I, application no. 6, in column 6 in the same line as SEQ ID NO. 8363 is depicted, mitochondrial is mentioned; which is operable linked, too.

In another embodiment thereof the expression cassette does not comprise an additional transit peptide in case in Table I, application no. 6, in column 6 in the same line as SEQ ID NO. 8363 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78970, or a homolog or fragment thereof, or a homolog thereof being depicted in Table I, application no. 6, column 8, in the same line as SEQ ID NO. 78970, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 78970 is depicted, plastidic is mentioned, or a mitochondrial transit peptide in case in Table I, application no. 6, in column 6 in the same line as SEQ ID NO. 78970 is depicted, mitochondrial is mentioned; which is operable linked, too.

In another embodiment thereof the expression cassette does not comprise an additional transit peptide in case in Table I, application no. 6, in column 6 in the same line as SEQ ID NO. 78970 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 79056, or a homolog or fragment thereof, or a homolog thereof being depicted in Table I, application no. 6, column 8, in the same line as SEQ ID NO. 79056, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 79056 is depicted, plastidic is mentioned, or a mitochondrial transit peptide in case in Table I, application no. 6, in column 6 in the same line as SEQ ID NO. 79056 is depicted, mitochondrial is mentioned; which is operable linked, too.

In another embodiment thereof the expression cassette does not comprise an additional transit peptide in case in Table I, application no. 6, in column 6 in the same line as SEQ ID NO. 79056 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 79217, or a homolog or fragment thereof, or a homolog thereof being depicted in Table I, application no. 6, column 8, in the same line as SEQ ID NO. 79217, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 79217 is depicted, plastidic is mentioned, or a mitochondrial transit peptide in case in Table I, application no. 6, in column 6 in the same line as SEQ ID NO. 79217 is depicted, mitochondrial is mentioned; which is operable linked, too.

In another embodiment thereof the expression cassette does not comprise an additional transit peptide in case in Table I, application no. 6, in column 6 in the same line as SEQ ID NO. 79217 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 104277, or a homolog or fragment thereof, or a homolog thereof being depicted in Table I, application no. 6, column 8, in the same line as SEQ ID NO. 104277, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 104277 is depicted, plastidic is mentioned, or a mitochondrial transit peptide in case in Table I, application no. 6, in column 6 in the same line as SEQ ID NO. 104277 is depicted, mitochondrial is mentioned; which is operable linked, too.

In another embodiment thereof the expression cassette does not comprise an additional transit peptide in case in Table I, application no. 6, in column 6 in the same line as SEQ ID NO. 104277 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38947, or a homolog or fragment thereof, or a homolog thereof being depicted in Table I, application no. 6, column 8, in the same line as SEQ ID NO. 38947, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 38947 is depicted, plastidic is mentioned, or a mitochondrial transit peptide in case in Table I, application no. 6, in column 6 in the same line as SEQ ID NO. 38947 is depicted, mitochondrial is mentioned; which is operable linked, too.

In another embodiment thereof the expression cassette does not comprise an additional transit peptide in case in Table I, application no. 6, in column 6 in the same line as SEQ ID NO. 38947 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 104616, or a homolog or fragment thereof, or a homolog thereof being depicted in Table I, application no. 6, column 8, in the same line as SEQ ID NO. 104616, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 104616 is depicted, plastidic is mentioned, or a mitochondrial transit peptide in case in Table I, application no. 6, in column 6 in the same line as SEQ ID NO. 104616 is depicted, mitochondrial is mentioned; which is operable linked, too.

In another embodiment thereof the expression cassette does not comprise an additional transit peptide in case in Table I, application no. 6, in column 6 in the same line as SEQ ID NO. 104616 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39255, or a homolog or fragment thereof, or a homolog thereof being depicted in Table I, application no. 6, column 8, in the same line as SEQ ID NO. 39255, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 39255 is depicted, plastidic is mentioned, or a mitochondrial transit peptide in case in Table I, application no. 6, in column 6 in the same line as SEQ ID NO. 39255 is depicted, mitochondrial is mentioned; which is operable linked, too.

In another embodiment thereof the expression cassette does not comprise an additional transit peptide in case in Table I, application no. 6, in column 6 in the same line as SEQ ID NO. 39255 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 9167, or a homolog or fragment thereof, or a homolog thereof being depicted in Table I, application no. 6, column 8, in the same line as SEQ ID NO. 9167, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 9167 is depicted, plastidic is mentioned, or a mitochondrial transit peptide in case in Table I, application no. 6, in column 6 in the same line as SEQ ID NO. 9167 is depicted, mitochondrial is mentioned; which is operable linked, too.

In another embodiment thereof the expression cassette does not comprise an additional transit peptide in case in Table I, application no. 6, in column 6 in the same line as SEQ ID NO. 9167 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 79627, or a homolog or fragment thereof, or a homolog thereof being depicted in Table I, application no. 6, column 8, in the same line as SEQ ID NO. 79627, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 79627 is depicted, plastidic is mentioned, or a mitochondrial transit peptide in case in Table I, application no. 6, in column 6 in the same line as SEQ ID NO. 79627 is depicted, mitochondrial is mentioned; which is operable linked, too.

In another embodiment thereof the expression cassette does not comprise an additional transit peptide in case in Table I, application no. 6, in column 6 in the same line as SEQ ID NO. 79627 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40665, or a homolog or fragment thereof, or a homolog thereof being depicted in Table I, application no. 6, column 8, in the same line as SEQ ID NO. 40665, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 40665 is depicted, plastidic is mentioned, or a mitochondrial transit peptide in case in Table I, application no. 6, in column 6 in the same line as SEQ ID NO. 40665 is depicted, mitochondrial is mentioned; which is operable linked, too.

In another embodiment thereof the expression cassette does not comprise an additional transit peptide in case in Table I, application no. 6, in column 6 in the same line as SEQ ID NO. 40665 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40726, or a homolog or fragment thereof, or a homolog thereof being depicted in Table I, application no. 6, column 8, in the same line as SEQ ID NO. 40726, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 40726 is depicted, plastidic is mentioned, or a mitochondrial transit peptide in case in Table I, application no. 6, in column 6 in the same line as SEQ ID NO. 40726 is depicted, mitochondrial is mentioned; which is operable linked, too.

In another embodiment thereof the expression cassette does not comprise an additional transit peptide in case in Table I, application no. 6, in column 6 in the same line as SEQ ID NO. 40726 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 9244, or a homolog or fragment thereof, or a homolog thereof being depicted in Table I, application no. 6, column 8, in the same line as SEQ ID NO. 9244, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 9244 is depicted, plastidic is mentioned, or a mitochondrial transit peptide in case in Table I, application no. 6, in column 6 in the same line as SEQ ID NO. 9244 is depicted, mitochondrial is mentioned; which is operable linked, too.

In another embodiment thereof the expression cassette does not comprise an additional transit peptide in case in Table I, application no. 6, in column 6 in the same line as SEQ ID NO. 9244 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 80756, or a homolog or fragment thereof, or a homolog thereof being depicted in Table I, application no. 6, column 8, in the same line as SEQ ID NO. 80756, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 80756 is depicted, plastidic is mentioned, or a mitochondrial transit peptide in case in Table I, application no. 6, in column 6 in the same line as SEQ ID NO. 80756 is depicted, mitochondrial is mentioned; which is operable linked, too.

In another embodiment thereof the expression cassette does not comprise an additional transit peptide in case in Table I, application no. 6, in column 6 in the same line as SEQ ID NO. 80756 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40741, or a homolog or fragment thereof, or a homolog thereof being depicted in Table I, application no. 6, column 8, in the same line as SEQ ID NO. 40741, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 40741 is depicted, plastidic is mentioned, or a mitochondrial transit peptide in case in Table I, application no. 6, in column 6 in the same line as SEQ ID NO. 40741 is depicted, mitochondrial is mentioned; which is operable linked, too.

In another embodiment thereof the expression cassette does not comprise an additional transit peptide in case in Table I, application no. 6, in column 6 in the same line as SEQ ID NO. 40741 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 80906, or a homolog or fragment thereof, or a homolog thereof being depicted in Table I, application no. 6, column 8, in the same line as SEQ ID NO. 80906, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 80906 is depicted, plastidic is mentioned, or a mitochondrial transit peptide in case in Table I, application no. 6, in column 6 in the same line as SEQ ID NO. 80906 is depicted, mitochondrial is mentioned; which is operable linked, too.

In another embodiment thereof the expression cassette does not comprise an additional transit peptide in case in Table I, application no. 6, in column 6 in the same line as SEQ ID NO. 80906 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40795, or a homolog or fragment thereof, or a homolog thereof being depicted in Table I, application no. 6, column 8, in the same line as SEQ ID NO. 40795, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 40795 is depicted, plastidic is mentioned, or a mitochondrial transit peptide in case in Table I, application no. 6, in column 6 in the same line as SEQ ID NO. 40795 is depicted, mitochondrial is mentioned; which is operable linked, too.

In another embodiment thereof the expression cassette does not comprise an additional transit peptide in case in Table I, application no. 6, in column 6 in the same line as SEQ ID NO. 40795 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 81935, or a homolog or fragment thereof, or a homolog thereof being depicted in Table I, application no. 6, column 8, in the same line as SEQ ID NO. 81935, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 81935 is depicted, plastidic is mentioned, or a mitochondrial transit peptide in case in Table I, application no. 6, in column 6 in the same line as SEQ ID NO. 81935 is depicted, mitochondrial is mentioned; which is operable linked, too.

In another embodiment thereof the expression cassette does not comprise an additional transit peptide in case in Table I, application no. 6, in column 6 in the same line as SEQ ID NO. 81935 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 41073, or a homolog or fragment thereof, or a homolog thereof being depicted in Table I, application no. 6, column 8, in the same line as SEQ ID NO. 41073, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 41073 is depicted, plastidic is mentioned, or a mitochondrial transit peptide in case in Table I, application no. 6, in column 6 in the same line as SEQ ID NO. 41073 is depicted, mitochondrial is mentioned; which is operable linked, too.

In another embodiment thereof the expression cassette does not comprise an additional transit peptide in case in Table I, application no. 6, in column 6 in the same line as SEQ ID NO. 41073 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 9333, or a homolog or fragment thereof, or a homolog thereof being depicted in Table I, application no. 6, column 8, in the same line as SEQ ID NO. 9333, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 9333 is depicted, plastidic is mentioned, or a mitochondrial transit peptide in case in Table I, application no. 6, in column 6 in the same line as SEQ ID NO. 9333 is depicted, mitochondrial is mentioned; which is operable linked, too.

In another embodiment thereof the expression cassette does not comprise an additional transit peptide in case in Table I, application no. 6, in column 6 in the same line as SEQ ID NO. 9333 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 42046, or a homolog or fragment thereof, or a homolog thereof being depicted in Table I, application no. 6, column 8, in the same line as SEQ ID NO. 42046, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 42046 is depicted, plastidic is mentioned, or a mitochondrial transit peptide in case in Table I, application no. 6, in column 6 in the same line as SEQ ID NO. 42046 is depicted, mitochondrial is mentioned; which is operable linked, too.

In another embodiment thereof the expression cassette does not comprise an additional transit peptide in case in Table I, application no. 6, in column 6 in the same line as SEQ ID NO. 42046 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 82424, or a homolog or fragment thereof, or a homolog thereof being depicted in Table I, application no. 6, column 8, in the same line as SEQ ID NO. 82424, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 82424 is depicted, plastidic is mentioned, or a mitochondrial transit peptide in case in Table I, application no. 6, in column 6 in the same line as SEQ ID NO. 82424 is depicted, mitochondrial is mentioned; which is operable linked, too.

In another embodiment thereof the expression cassette does not comprise an additional transit peptide in case in Table I, application no. 6, in column 6 in the same line as SEQ ID NO. 82424 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 104660, or a homolog or fragment thereof, or a homolog thereof being depicted in Table I, application no. 6, column 8, in the same line as SEQ ID NO. 104660, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 104660 is depicted, plastidic is mentioned, or a mitochondrial transit peptide in case in Table I, application no. 6, in column 6 in the same line as SEQ ID NO. 104660 is depicted, mitochondrial is mentioned; which is operable linked, too.

In another embodiment thereof the expression cassette does not comprise an additional transit peptide in case in Table I, application no. 6, in column 6 in the same line as SEQ ID NO. 104660 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 104871, or a homolog or fragment thereof, or a homolog thereof being depicted in Table I, application no. 6, column 8, in the same line as SEQ ID NO. 104871, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 104871 is depicted, plastidic is mentioned, or a mitochondrial transit peptide in case in Table I, application no. 6, in column 6 in the same line as SEQ ID NO. 104871 is depicted, mitochondrial is mentioned; which is operable linked, too.

In another embodiment thereof the expression cassette does not comprise an additional transit peptide in case in Table I, application no. 6, in column 6 in the same line as SEQ ID NO. 104871 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 9492, or a homolog or fragment thereof, or a homolog thereof being depicted in Table I, application no. 6, column 8, in the same line as SEQ ID NO. 9492, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 9492 is depicted, plastidic is mentioned, or a mitochondrial transit peptide in case in Table I, application no. 6, in column 6 in the same line as SEQ ID NO. 9492 is depicted, mitochondrial is mentioned; which is operable linked, too.

In another embodiment thereof the expression cassette does not comprise an additional transit peptide in case in Table I, application no. 6, in column 6 in the same line as SEQ ID NO. 9492 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 82572, or a homolog or fragment thereof, or a homolog thereof being depicted in Table I, application no. 6, column 8, in the same line as SEQ ID NO. 82572, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 82572 is depicted, plastidic is mentioned, or a mitochondrial transit peptide in case in Table I, application no. 6, in column 6 in the same line as SEQ ID NO. 82572 is depicted, mitochondrial is mentioned; which is operable linked, too.

In another embodiment thereof the expression cassette does not comprise an additional transit peptide in case in Table I, application no. 6, in column 6 in the same line as SEQ ID NO. 82572 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 94051, or a homolog or fragment thereof, or a homolog thereof being depicted in Table I, application no. 6, column 8, in the same line as SEQ ID NO. 94051, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 94051 is depicted, plastidic is mentioned, or a mitochondrial transit peptide in case in Table I, application no. 6, in column 6 in the same line as SEQ ID NO. 94051 is depicted, mitochondrial is mentioned; which is operable linked, too.

In another embodiment thereof the expression cassette does not comprise an additional transit peptide in case in Table I, application no. 6, in column 6 in the same line as SEQ ID NO. 94051 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 105332, or a homolog or fragment thereof, or a homolog thereof being depicted in Table I, application no. 6, column 8, in the same line as SEQ ID NO. 105332, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 105332 is depicted, plastidic is mentioned, or a mitochondrial transit peptide in case in Table I, application no. 6, in column 6 in the same line as SEQ ID NO. 105332 is depicted, mitochondrial is mentioned; which is operable linked, too.

In another embodiment thereof the expression cassette does not comprise an additional transit peptide in case in Table I, application no. 6, in column 6 in the same line as SEQ ID NO. 105332 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 10172, or a homolog or fragment thereof, or a homolog thereof being depicted in Table I, application no. 6, column 8, in the same line as SEQ ID NO. 10172, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 10172 is depicted, plastidic is mentioned, or a mitochondrial transit peptide in case in Table I, application no. 6, in column 6 in the same line as SEQ ID NO. 10172 is depicted, mitochondrial is mentioned; which is operable linked, too.

In another embodiment thereof the expression cassette does not comprise an additional transit peptide in case in Table I, application no. 6, in column 6 in the same line as SEQ ID NO. 10172 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 42931, or a homolog or fragment thereof, or a homolog thereof being depicted in Table I, application no. 6, column 8, in the same line as SEQ ID NO. 42931, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 42931 is depicted, plastidic is mentioned, or a mitochondrial transit peptide in case in Table I, application no. 6, in column 6 in the same line as SEQ ID NO. 42931 is depicted, mitochondrial is mentioned; which is operable linked, too.

In another embodiment thereof the expression cassette does not comprise an additional transit peptide in case in Table I, application no. 6, in column 6 in the same line as SEQ ID NO. 42931 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 83629, or a homolog or fragment thereof, or a homolog thereof being depicted in Table I, application no. 6, column 8, in the same line as SEQ ID NO. 83629, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 83629 is depicted, plastidic is mentioned, or a mitochondrial transit peptide in case in Table I, application no. 6, in column 6 in the same line as SEQ ID NO. 83629 is depicted, mitochondrial is mentioned; which is operable linked, too.

In another embodiment thereof the expression cassette does not comprise an additional transit peptide in case in Table I, application no. 6, in column 6 in the same line as SEQ ID NO. 83629 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 105354, or a homolog or fragment thereof, or a homolog thereof being depicted in Table I, application no. 6, column 8, in the same line as SEQ ID NO. 105354, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 105354 is depicted, plastidic is mentioned, or a mitochondrial transit peptide in case in Table I, application no. 6, in column 6 in the same line as SEQ ID NO. 105354 is depicted, mitochondrial is mentioned; which is operable linked, too.

In another embodiment thereof the expression cassette does not comprise an additional transit peptide in case in Table I, application no. 6, in column 6 in the same line as SEQ ID NO. 105354 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 10252, or a homolog or fragment thereof, or a homolog thereof being depicted in Table I, application no. 6, column 8, in the same line as SEQ ID NO. 10252, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 10252 is depicted, plastidic is mentioned, or a mitochondrial transit peptide in case in Table I, application no. 6, in column 6 in the same line as SEQ ID NO. 10252 is depicted, mitochondrial is mentioned; which is operable linked, too.

In another embodiment thereof the expression cassette does not comprise an additional transit peptide in case in Table I, application no. 6, in column 6 in the same line as SEQ ID NO. 10252 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 94386, or a homolog or fragment thereof, or a homolog thereof being depicted in Table I, application no. 6, column 8, in the same line as SEQ ID NO. 94386, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 94386 is depicted, plastidic is mentioned, or a mitochondrial transit peptide in case in Table I, application no. 6, in column 6 in the same line as SEQ ID NO. 94386 is depicted, mitochondrial is mentioned; which is operable linked, too.

In another embodiment thereof the expression cassette does not comprise an additional transit peptide in case in Table I, application no. 6, in column 6 in the same line as SEQ ID NO. 94386 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 94386, or a homolog or fragment thereof, or a homolog thereof being depicted in Table I, application no. 6, column 8, in the same line as SEQ ID NO. 94386, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 94386 is depicted, plastidic is mentioned, or a mitochondrial transit peptide in case in Table I, application no. 6, in column 6 in the same line as SEQ ID NO. 94386 is depicted, mitochondrial is mentioned; which is operable linked, too.

In another embodiment thereof the expression cassette does not comprise an additional transit peptide in case in Table I, application no. 6, in column 6 in the same line as SEQ ID NO. 94386 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 43839, or a homolog or fragment thereof, or a homolog thereof being depicted in Table I, application no. 6, column 8, in the same line as SEQ ID NO. 43839, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 43839 is depicted, plastidic is mentioned, or a mitochondrial transit peptide in case in Table I, application no. 6, in column 6 in the same line as SEQ ID NO. 43839 is depicted, mitochondrial is mentioned; which is operable linked, too.

In another embodiment thereof the expression cassette does not comprise an additional transit peptide in case in Table I, application no. 6, in column 6 in the same line as SEQ ID NO. 43839 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 105602, or a homolog or fragment thereof, or a homolog thereof being depicted in Table I, application no. 6, column 8, in the same line as SEQ ID NO. 105602, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 105602 is depicted, plastidic is mentioned, or a mitochondrial transit peptide in case in Table I, application no. 6, in column 6 in the same line as SEQ ID NO. 105602 is depicted, mitochondrial is mentioned; which is operable linked, too.

In another embodiment thereof the expression cassette does not comprise an additional transit peptide in case in Table I, application no. 6, in column 6 in the same line as SEQ ID NO. 105602 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 105659, or a homolog or fragment thereof, or a homolog thereof being depicted in Table I, application no. 6, column 8, in the same line as SEQ ID NO. 105659, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 105659 is depicted, plastidic is mentioned, or a mitochondrial transit peptide in case in Table I, application no. 6, in column 6 in the same line as SEQ ID NO. 105659 is depicted, mitochondrial is mentioned; which is operable linked, too.

In another embodiment thereof the expression cassette does not comprise an additional transit peptide in case in Table I, application no. 6, in column 6 in the same line as SEQ ID NO. 105659 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 44372, or a homolog or fragment thereof, or a homolog thereof being depicted in Table I, application no. 6, column 8, in the same line as SEQ ID NO. 44372, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 44372 is depicted, plastidic is mentioned, or a mitochondrial transit peptide in case in Table I, application no. 6, in column 6 in the same line as SEQ ID NO. 44372 is depicted, mitochondrial is mentioned; which is operable linked, too.

In another embodiment thereof the expression cassette does not comprise an additional transit peptide in case in Table I, application no. 6, in column 6 in the same line as SEQ ID NO. 44372 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 10740, or a homolog or fragment thereof, or a homolog thereof being depicted in Table I, application no. 6, column 8, in the same line as SEQ ID NO. 10740, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 10740 is depicted, plastidic is mentioned, or a mitochondrial transit peptide in case in Table I, application no. 6, in column 6 in the same line as SEQ ID NO. 10740 is depicted, mitochondrial is mentioned; which is operable linked, too.

In another embodiment thereof the expression cassette does not comprise an additional transit peptide in case in Table I, application no. 6, in column 6 in the same line as SEQ ID NO. 10740 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 106532, or a homolog or fragment thereof, or a homolog thereof being depicted in Table I, application no. 6, column 8, in the same line as SEQ ID NO. 106532, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 106532 is depicted, plastidic is mentioned, or a mitochondrial transit peptide in case in Table I, application no. 6, in column 6 in the same line as SEQ ID NO. 106532 is depicted, mitochondrial is mentioned; which is operable linked, too.

In another embodiment thereof the expression cassette does not comprise an additional transit peptide in case in Table I, application no. 6, in column 6 in the same line as SEQ ID NO. 106532 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 94542, or a homolog or fragment thereof, or a homolog thereof being depicted in Table I, application no. 6, column 8, in the same line as SEQ ID NO. 94542, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 94542 is depicted, plastidic is mentioned, or a mitochondrial transit peptide in case in Table I, application no. 6, in column 6 in the same line as SEQ ID NO. 94542 is depicted, mitochondrial is mentioned; which is operable linked, too.

In another embodiment thereof the expression cassette does not comprise an additional transit peptide in case in Table I, application no. 6, in column 6 in the same line as SEQ ID NO. 94542 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 45321, or a homolog or fragment thereof, or a homolog thereof being depicted in Table I, application no. 6, column 8, in the same line as SEQ ID NO. 45321, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 45321 is depicted, plastidic is mentioned, or a mitochondrial transit peptide in case in Table I, application no. 6, in column 6 in the same line as SEQ ID NO. 45321 is depicted, mitochondrial is mentioned; which is operable linked, too.

In another embodiment thereof the expression cassette does not comprise an additional transit peptide in case in Table I, application no. 6, in column 6 in the same line as SEQ ID NO. 45321 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 106538, or a homolog or fragment thereof, or a homolog thereof being depicted in Table I, application no. 6, column 8, in the same line as SEQ ID NO. 106538, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 106538 is depicted, plastidic is mentioned, or a mitochondrial transit peptide in case in Table I, application no. 6, in column 6 in the same line as SEQ ID NO. 106538 is depicted, mitochondrial is mentioned; which is operable linked, too.

In another embodiment thereof the expression cassette does not comprise an additional transit peptide in case in Table I, application no. 6, in column 6 in the same line as SEQ ID NO. 106538 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 45394, or a homolog or fragment thereof, or a homolog thereof being depicted in Table I, application no. 6, column 8, in the same line as SEQ ID NO. 45394, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 45394 is depicted, plastidic is mentioned, or a mitochondrial transit peptide in case in Table I, application no. 6, in column 6 in the same line as SEQ ID NO. 45394 is depicted, mitochondrial is mentioned; which is operable linked, too.

In another embodiment thereof the expression cassette does not comprise an additional transit peptide in case in Table I, application no. 6, in column 6 in the same line as SEQ ID NO. 45394 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 45556, or a homolog or fragment thereof, or a homolog thereof being depicted in Table I, application no. 6, column 8, in the same line as SEQ ID NO. 45556, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 45556 is depicted, plastidic is mentioned, or a mitochondrial transit peptide in case in Table I, application no. 6, in column 6 in the same line as SEQ ID NO. 45556 is depicted, mitochondrial is mentioned; which is operable linked, too.

In another embodiment thereof the expression cassette does not comprise an additional transit peptide in case in Table I, application no. 6, in column 6 in the same line as SEQ ID NO. 45556 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 45757, or a homolog or fragment thereof, or a homolog thereof being depicted in Table I, application no. 6, column 8, in the same line as SEQ ID NO. 45757, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 45757 is depicted, plastidic is mentioned, or a mitochondrial transit peptide in case in Table I, application no. 6, in column 6 in the same line as SEQ ID NO. 45757 is depicted, mitochondrial is mentioned; which is operable linked, too.

In another embodiment thereof the expression cassette does not comprise an additional transit peptide in case in Table I, application no. 6, in column 6 in the same line as SEQ ID NO. 45757 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 45795, or a homolog or fragment thereof, or a homolog thereof being depicted in Table I, application no. 6, column 8, in the same line as SEQ ID NO. 45795, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 45795 is depicted, plastidic is mentioned, or a mitochondrial transit peptide in case in Table I, application no. 6, in column 6 in the same line as SEQ ID NO. 45795 is depicted, mitochondrial is mentioned; which is operable linked, too.

In another embodiment thereof the expression cassette does not comprise an additional transit peptide in case in Table I, application no. 6, in column 6 in the same line as SEQ ID NO. 45795 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 10811, or a homolog or fragment thereof, or a homolog thereof being depicted in Table I, application no. 6, column 8, in the same line as SEQ ID NO. 10811, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 10811 is depicted, plastidic is mentioned, or a mitochondrial transit peptide in case in Table I, application no. 6, in column 6 in the same line as SEQ ID NO. 10811 is depicted, mitochondrial is mentioned; which is operable linked, too.

In another embodiment thereof the expression cassette does not comprise an additional transit peptide in case in Table I, application no. 6, in column 6 in the same line as SEQ ID NO. 10811 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 45897, or a homolog or fragment thereof, or a homolog thereof being depicted in Table I, application no. 6, column 8, in the same line as SEQ ID NO. 45897, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 45897 is depicted, plastidic is mentioned, or a mitochondrial transit peptide in case in Table I, application no. 6, in column 6 in the same line as SEQ ID NO. 45897 is depicted, mitochondrial is mentioned; which is operable linked, too.

In another embodiment thereof the expression cassette does not comprise an additional transit peptide in case in Table I, application no. 6, in column 6 in the same line as SEQ ID NO. 45897 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 106688, or a homolog or fragment thereof, or a homolog thereof being depicted in Table I, application no. 6, column 8, in the same line as SEQ ID NO. 106688, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 106688 is depicted, plastidic is mentioned, or a mitochondrial transit peptide in case in Table I, application no. 6, in column 6 in the same line as SEQ ID NO. 106688 is depicted, mitochondrial is mentioned; which is operable linked, too.

In another embodiment thereof the expression cassette does not comprise an additional transit peptide in case in Table I, application no. 6, in column 6 in the same line as SEQ ID NO. 106688 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 46515, or a homolog or fragment thereof, or a homolog thereof being depicted in Table I, application no. 6, column 8, in the same line as SEQ ID NO. 46515, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 46515 is depicted, plastidic is mentioned, or a mitochondrial transit peptide in case in Table I, application no. 6, in column 6 in the same line as SEQ ID NO. 46515 is depicted, mitochondrial is mentioned; which is operable linked, too.

In another embodiment thereof the expression cassette does not comprise an additional transit peptide in case in Table I, application no. 6, in column 6 in the same line as SEQ ID NO. 46515 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 84198, or a homolog or fragment thereof, or a homolog thereof being depicted in Table I, application no. 6, column 8, in the same line as SEQ ID NO. 84198, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 84198 is depicted, plastidic is mentioned, or a mitochondrial transit peptide in case in Table I, application no. 6, in column 6 in the same line as SEQ ID NO. 84198 is depicted, mitochondrial is mentioned; which is operable linked, too.

In another embodiment thereof the expression cassette does not comprise an additional transit peptide in case in Table I, application no. 6, in column 6 in the same line as SEQ ID NO. 84198 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 46850, or a homolog or fragment thereof, or a homolog thereof being depicted in Table I, application no. 6, column 8, in the same line as SEQ ID NO. 46850, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 46850 is depicted, plastidic is mentioned, or a mitochondrial transit peptide in case in Table I, application no. 6, in column 6 in the same line as SEQ ID NO. 46850 is depicted, mitochondrial is mentioned; which is operable linked, too.

In another embodiment thereof the expression cassette does not comprise an additional transit peptide in case in Table I, application no. 6, in column 6 in the same line as SEQ ID NO. 46850 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 47076, or a homolog or fragment thereof, or a homolog thereof being depicted in Table I, application no. 6, column 8, in the same line as SEQ ID NO. 47076, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 47076 is depicted, plastidic is mentioned, or a mitochondrial transit peptide in case in Table I, application no. 6, in column 6 in the same line as SEQ ID NO. 47076 is depicted, mitochondrial is mentioned; which is operable linked, too.

In another embodiment thereof the expression cassette does not comprise an additional transit peptide in case in Table I, application no. 6, in column 6 in the same line as SEQ ID NO. 47076 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 84265, or a homolog or fragment thereof, or a homolog thereof being depicted in Table I, application no. 6, column 8, in the same line as SEQ ID NO. 84265, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 84265 is depicted, plastidic is mentioned, or a mitochondrial transit peptide in case in Table I, application no. 6, in column 6 in the same line as SEQ ID NO. 84265 is depicted, mitochondrial is mentioned; which is operable linked, too.

In another embodiment thereof the expression cassette does not comprise an additional transit peptide in case in Table I, application no. 6, in column 6 in the same line as SEQ ID NO. 84265 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 84441, or a homolog or fragment thereof, or a homolog thereof being depicted in Table I, application no. 6, column 8, in the same line as SEQ ID NO. 84441, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 84441 is depicted, plastidic is mentioned, or a mitochondrial transit peptide in case in Table I, application no. 6, in column 6 in the same line as SEQ ID NO. 84441 is depicted, mitochondrial is mentioned; which is operable linked, too.

In another embodiment thereof the expression cassette does not comprise an additional transit peptide in case in Table I, application no. 6, in column 6 in the same line as SEQ ID NO. 84441 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 49143, or a homolog or fragment thereof, or a homolog thereof being depicted in Table I, application no. 6, column 8, in the same line as SEQ ID NO. 49143, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 49143 is depicted, plastidic is mentioned, or a mitochondrial transit peptide in case in Table I, application no. 6, in column 6 in the same line as SEQ ID NO. 49143 is depicted, mitochondrial is mentioned; which is operable linked, too.

In another embodiment thereof the expression cassette does not comprise an additional transit peptide in case in Table I, application no. 6, in column 6 in the same line as SEQ ID NO. 49143 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 49828, or a homolog or fragment thereof, or a homolog thereof being depicted in Table I, application no. 6, column 8, in the same line as SEQ ID NO. 49828, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 49828 is depicted, plastidic is mentioned, or a mitochondrial transit peptide in case in Table I, application no. 6, in column 6 in the same line as SEQ ID NO. 49828 is depicted, mitochondrial is mentioned; which is operable linked, too.

In another embodiment thereof the expression cassette does not comprise an additional transit peptide in case in Table I, application no. 6, in column 6 in the same line as SEQ ID NO. 49828 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 106920, or a homolog or fragment thereof, or a homolog thereof being depicted in Table I, application no. 6, column 8, in the same line as SEQ ID NO. 106920, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 106920 is depicted, plastidic is mentioned, or a mitochondrial transit peptide in case in Table I, application no. 6, in column 6 in the same line as SEQ ID NO. 106920 is depicted, mitochondrial is mentioned; which is operable linked, too.

In another embodiment thereof the expression cassette does not comprise an additional transit peptide in case in Table I, application no. 6, in column 6 in the same line as SEQ ID NO. 106920 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 50104, or a homolog or fragment thereof, or a homolog thereof being depicted in Table I, application no. 6, column 8, in the same line as SEQ ID NO. 50104, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 50104 is depicted, plastidic is mentioned, or a mitochondrial transit peptide in case in Table I, application no. 6, in column 6 in the same line as SEQ ID NO. 50104 is depicted, mitochondrial is mentioned; which is operable linked, too.

In another embodiment thereof the expression cassette does not comprise an additional transit peptide in case in Table I, application no. 6, in column 6 in the same line as SEQ ID NO. 50104 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 50339, or a homolog or fragment thereof, or a homolog thereof being depicted in Table I, application no. 6, column 8, in the same line as SEQ ID NO. 50339, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 50339 is depicted, plastidic is mentioned, or a mitochondrial transit peptide in case in Table I, application no. 6, in column 6 in the same line as SEQ ID NO. 50339 is depicted, mitochondrial is mentioned; which is operable linked, too.

In another embodiment thereof the expression cassette does not comprise an additional transit peptide in case in Table I, application no. 6, in column 6 in the same line as SEQ ID NO. 50339 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 51198, or a homolog or fragment thereof, or a homolog thereof being depicted in Table I, application no. 6, column 8, in the same line as SEQ ID NO. 51198, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 51198 is depicted, plastidic is mentioned, or a mitochondrial transit peptide in case in Table I, application no. 6, in column 6 in the same line as SEQ ID NO. 51198 is depicted, mitochondrial is mentioned; which is operable linked, too.

In another embodiment thereof the expression cassette does not comprise an additional transit peptide in case in Table I, application no. 6, in column 6 in the same line as SEQ ID NO. 51198 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 101707, or a homolog or fragment thereof, or a homolog thereof being depicted in Table I, application no. 6, column 8, in the same line as SEQ ID NO. 101707, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 101707 is depicted, plastidic is mentioned, or a mitochondrial transit peptide in case in Table I, application no. 6, in column 6 in the same line as SEQ ID NO. 101707 is depicted, mitochondrial is mentioned; which is operable linked, too.

In another embodiment thereof the expression cassette does not comprise an additional transit peptide in case in Table I, application no. 6, in column 6 in the same line as SEQ ID NO. 101707 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 85723, or a homolog or fragment thereof, or a homolog thereof being depicted in Table I, application no. 6, column 8, in the same line as SEQ ID NO. 85723, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 85723 is depicted, plastidic is mentioned, or a mitochondrial transit peptide in case in Table I, application no. 6, in column 6 in the same line as SEQ ID NO. 85723 is depicted, mitochondrial is mentioned; which is operable linked, too.

In another embodiment thereof the expression cassette does not comprise an additional transit peptide in case in Table I, application no. 6, in column 6 in the same line as SEQ ID NO. 85723 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 53456, or a homolog or fragment thereof, or a homolog thereof being depicted in Table I, application no. 6, column 8, in the same line as SEQ ID NO. 53456, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 53456 is depicted, plastidic is mentioned, or a mitochondrial transit peptide in case in Table I, application no. 6, in column 6 in the same line as SEQ ID NO. 53456 is depicted, mitochondrial is mentioned; which is operable linked, too.

In another embodiment thereof the expression cassette does not comprise an additional transit peptide in case in Table I, application no. 6, in column 6 in the same line as SEQ ID NO. 53456 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 107100, or a homolog or fragment thereof, or a homolog thereof being depicted in Table I, application no. 6, column 8, in the same line as SEQ ID NO. 107100, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 107100 is depicted, plastidic is mentioned, or a mitochondrial transit peptide in case in Table I, application no. 6, in column 6 in the same line as SEQ ID NO. 107100 is depicted, mitochondrial is mentioned; which is operable linked, too.

In another embodiment thereof the expression cassette does not comprise an additional transit peptide in case in Table I, application no. 6, in column 6 in the same line as SEQ ID NO. 107100 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 95047, or a homolog or fragment thereof, or a homolog thereof being depicted in Table I, application no. 6, column 8, in the same line as SEQ ID NO. 95047, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 95047 is depicted, plastidic is mentioned, or a mitochondrial transit peptide in case in Table I, application no. 6, in column 6 in the same line as SEQ ID NO. 95047 is depicted, mitochondrial is mentioned; which is operable linked, too.

In another embodiment thereof the expression cassette does not comprise an additional transit peptide in case in Table I, application no. 6, in column 6 in the same line as SEQ ID NO. 95047 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 55379, or a homolog or fragment thereof, or a homolog thereof being depicted in Table I, application no. 6, column 8, in the same line as SEQ ID NO. 55379, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 55379 is depicted, plastidic is mentioned, or a mitochondrial transit peptide in case in Table I, application no. 6, in column 6 in the same line as SEQ ID NO. 55379 is depicted, mitochondrial is mentioned; which is operable linked, too.

In another embodiment thereof the expression cassette does not comprise an additional transit peptide in case in Table I, application no. 6, in column 6 in the same line as SEQ ID NO. 55379 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 85808, or a homolog or fragment thereof, or a homolog thereof being depicted in Table I, application no. 6, column 8, in the same line as SEQ ID NO. 85808, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 85808 is depicted, plastidic is mentioned, or a mitochondrial transit peptide in case in Table I, application no. 6, in column 6 in the same line as SEQ ID NO. 85808 is depicted, mitochondrial is mentioned; which is operable linked, too.

In another embodiment thereof the expression cassette does not comprise an additional transit peptide in case in Table I, application no. 6, in column 6 in the same line as SEQ ID NO. 85808 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 86447, or a homolog or fragment thereof, or a homolog thereof being depicted in Table I, application no. 6, column 8, in the same line as SEQ ID NO. 86447, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 86447 is depicted, plastidic is mentioned, or a mitochondrial transit peptide in case in Table I, application no. 6, in column 6 in the same line as SEQ ID NO. 86447 is depicted, mitochondrial is mentioned; which is operable linked, too.

In another embodiment thereof the expression cassette does not comprise an additional transit peptide in case in Table I, application no. 6, in column 6 in the same line as SEQ ID NO. 86447 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 95527, or a homolog or fragment thereof, or a homolog thereof being depicted in Table I, application no. 6, column 8, in the same line as SEQ ID NO. 95527, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 95527 is depicted, plastidic is mentioned, or a mitochondrial transit peptide in case in Table I, application no. 6, in column 6 in the same line as SEQ ID NO. 95527 is depicted, mitochondrial is mentioned; which is operable linked, too.

In another embodiment thereof the expression cassette does not comprise an additional transit peptide in case in Table I, application no. 6, in column 6 in the same line as SEQ ID NO. 95527 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 86540, or a homolog or fragment thereof, or a homolog thereof being depicted in Table I, application no. 6, column 8, in the same line as SEQ ID NO. 86540, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 86540 is depicted, plastidic is mentioned, or a mitochondrial transit peptide in case in Table I, application no. 6, in column 6 in the same line as SEQ ID NO. 86540 is depicted, mitochondrial is mentioned; which is operable linked, too.

In another embodiment thereof the expression cassette does not comprise an additional transit peptide in case in Table I, application no. 6, in column 6 in the same line as SEQ ID NO. 86540 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 87397, or a homolog or fragment thereof, or a homolog thereof being depicted in Table I, application no. 6, column 8, in the same line as SEQ ID NO. 87397, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 87397 is depicted, plastidic is mentioned, or a mitochondrial transit peptide in case in Table I, application no. 6, in column 6 in the same line as SEQ ID NO. 87397 is depicted, mitochondrial is mentioned; which is operable linked, too.

In another embodiment thereof the expression cassette does not comprise an additional transit peptide in case in Table I, application no. 6, in column 6 in the same line as SEQ ID NO. 87397 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 57235, or a homolog or fragment thereof, or a homolog thereof being depicted in Table I, application no. 6, column 8, in the same line as SEQ ID NO. 57235, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 57235 is depicted, plastidic is mentioned, or a mitochondrial transit peptide in case in Table I, application no. 6, in column 6 in the same line as SEQ ID NO. 57235 is depicted, mitochondrial is mentioned; which is operable linked, too.

In another embodiment thereof the expression cassette does not comprise an additional transit peptide in case in Table I, application no. 6, in column 6 in the same line as SEQ ID NO. 57235 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 58668, or a homolog or fragment thereof, or a homolog thereof being depicted in Table I, application no. 6, column 8, in the same line as SEQ ID NO. 58668, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 58668 is depicted, plastidic is mentioned, or a mitochondrial transit peptide in case in Table I, application no. 6, in column 6 in the same line as SEQ ID NO. 58668 is depicted, mitochondrial is mentioned; which is operable linked, too.

In another embodiment thereof the expression cassette does not comprise an additional transit peptide in case in Table I, application no. 6, in column 6 in the same line as SEQ ID NO. 58668 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 88158, or a homolog or fragment thereof, or a homolog thereof being depicted in Table I, application no. 6, column 8, in the same line as SEQ ID NO. 88158, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 88158 is depicted, plastidic is mentioned, or a mitochondrial transit peptide in case in Table I, application no. 6, in column 6 in the same line as SEQ ID NO. 88158 is depicted, mitochondrial is mentioned; which is operable linked, too.

In another embodiment thereof the expression cassette does not comprise an additional transit peptide in case in Table I, application no. 6, in column 6 in the same line as SEQ ID NO. 88158 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 107145, or a homolog or fragment thereof, or a homolog thereof being depicted in Table I, application no. 6, column 8, in the same line as SEQ ID NO. 107145, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 107145 is depicted, plastidic is mentioned, or a mitochondrial transit peptide in case in Table I, application no. 6, in column 6 in the same line as SEQ ID NO. 107145 is depicted, mitochondrial is mentioned; which is operable linked, too.

In another embodiment thereof the expression cassette does not comprise an additional transit peptide in case in Table I, application no. 6, in column 6 in the same line as SEQ ID NO. 107145 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 58731, or a homolog or fragment thereof, or a homolog thereof being depicted in Table I, application no. 6, column 8, in the same line as SEQ ID NO. 58731, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 58731 is depicted, plastidic is mentioned, or a mitochondrial transit peptide in case in Table I, application no. 6, in column 6 in the same line as SEQ ID NO. 58731 is depicted, mitochondrial is mentioned; which is operable linked, too.

In another embodiment thereof the expression cassette does not comprise an additional transit peptide in case in Table I, application no. 6, in column 6 in the same line as SEQ ID NO. 58731 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12140, or a homolog or fragment thereof, or a homolog thereof being depicted in Table I, application no. 6, column 8, in the same line as SEQ ID NO. 12140, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 12140 is depicted, plastidic is mentioned, or a mitochondrial transit peptide in case in Table I, application no. 6, in column 6 in the same line as SEQ ID NO. 12140 is depicted, mitochondrial is mentioned; which is operable linked, too.

In another embodiment thereof the expression cassette does not comprise an additional transit peptide in case in Table I, application no. 6, in column 6 in the same line as SEQ ID NO. 12140 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 59041, or a homolog or fragment thereof, or a homolog thereof being depicted in Table I, application no. 6, column 8, in the same line as SEQ ID NO. 59041, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 59041 is depicted, plastidic is mentioned, or a mitochondrial transit peptide in case in Table I, application no. 6, in column 6 in the same line as SEQ ID NO. 59041 is depicted, mitochondrial is mentioned; which is operable linked, too.

In another embodiment thereof the expression cassette does not comprise an additional transit peptide in case in Table I, application no. 6, in column 6 in the same line as SEQ ID NO. 59041 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12341, or a homolog or fragment thereof, or a homolog thereof being depicted in Table I, application no. 6, column 8, in the same line as SEQ ID NO. 12341, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 12341 is depicted, plastidic is mentioned, or a mitochondrial transit peptide in case in Table I, application no. 6, in column 6 in the same line as SEQ ID NO. 12341 is depicted, mitochondrial is mentioned; which is operable linked, too.

In another embodiment thereof the expression cassette does not comprise an additional transit peptide in case in Table I, application no. 6, in column 6 in the same line as SEQ ID NO. 12341 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 59370, or a homolog or fragment thereof, or a homolog thereof being depicted in Table I, application no. 6, column 8, in the same line as SEQ ID NO. 59370, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 59370 is depicted, plastidic is mentioned, or a mitochondrial transit peptide in case in Table I, application no. 6, in column 6 in the same line as SEQ ID NO. 59370 is depicted, mitochondrial is mentioned; which is operable linked, too.

In another embodiment thereof the expression cassette does not comprise an additional transit peptide in case in Table I, application no. 6, in column 6 in the same line as SEQ ID NO. 59370 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12698, or a homolog or fragment thereof, or a homolog thereof being depicted in Table I, application no. 6, column 8, in the same line as SEQ ID NO. 12698, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 12698 is depicted, plastidic is mentioned, or a mitochondrial transit peptide in case in Table I, application no. 6, in column 6 in the same line as SEQ ID NO. 12698 is depicted, mitochondrial is mentioned; which is operable linked, too.

In another embodiment thereof the expression cassette does not comprise an additional transit peptide in case in Table I, application no. 6, in column 6 in the same line as SEQ ID NO. 12698 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 107556, or a homolog or fragment thereof, or a homolog thereof being depicted in Table I, application no. 6, column 8, in the same line as SEQ ID NO. 107556, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 107556 is depicted, plastidic is mentioned, or a mitochondrial transit peptide in case in Table I, application no. 6, in column 6 in the same line as SEQ ID NO. 107556 is depicted, mitochondrial is mentioned; which is operable linked, too.

In another embodiment thereof the expression cassette does not comprise an additional transit peptide in case in Table I, application no. 6, in column 6 in the same line as SEQ ID NO. 107556 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 62079, or a homolog or fragment thereof, or a homolog thereof being depicted in Table I, application no. 6, column 8, in the same line as SEQ ID NO. 62079, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 62079 is depicted, plastidic is mentioned, or a mitochondrial transit peptide in case in Table I, application no. 6, in column 6 in the same line as SEQ ID NO. 62079 is depicted, mitochondrial is mentioned; which is operable linked, too.

In another embodiment thereof the expression cassette does not comprise an additional transit peptide in case in Table I, application no. 6, in column 6 in the same line as SEQ ID NO. 62079 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12974, or a homolog or fragment thereof, or a homolog thereof being depicted in Table I, application no. 6, column 8, in the same line as SEQ ID NO. 12974, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 12974 is depicted, plastidic is mentioned, or a mitochondrial transit peptide in case in Table I, application no. 6, in column 6 in the same line as SEQ ID NO. 12974 is depicted, mitochondrial is mentioned; which is operable linked, too.

In another embodiment thereof the expression cassette does not comprise an additional transit peptide in case in Table I, application no. 6, in column 6 in the same line as SEQ ID NO. 12974 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 108092, or a homolog or fragment thereof, or a homolog thereof being depicted in Table I, application no. 6, column 8, in the same line as SEQ ID NO. 108092, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 108092 is depicted, plastidic is mentioned, or a mitochondrial transit peptide in case in Table I, application no. 6, in column 6 in the same line as SEQ ID NO. 108092 is depicted, mitochondrial is mentioned; which is operable linked, too.

In another embodiment thereof the expression cassette does not comprise an additional transit peptide in case in Table I, application no. 6, in column 6 in the same line as SEQ ID NO. 108092 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 62717, or a homolog or fragment thereof, or a homolog thereof being depicted in Table I, application no. 6, column 8, in the same line as SEQ ID NO. 62717, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 62717 is depicted, plastidic is mentioned, or a mitochondrial transit peptide in case in Table I, application no. 6, in column 6 in the same line as SEQ ID NO. 62717 is depicted, mitochondrial is mentioned; which is operable linked, too.

In another embodiment thereof the expression cassette does not comprise an additional transit peptide in case in Table I, application no. 6, in column 6 in the same line as SEQ ID NO. 62717 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 108113, or a homolog or fragment thereof, or a homolog thereof being depicted in Table I, application no. 6, column 8, in the same line as SEQ ID NO. 108113, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 108113 is depicted, plastidic is mentioned, or a mitochondrial transit peptide in case in Table I, application no. 6, in column 6 in the same line as SEQ ID NO. 108113 is depicted, mitochondrial is mentioned; which is operable linked, too.

In another embodiment thereof the expression cassette does not comprise an additional transit peptide in case in Table I, application no. 6, in column 6 in the same line as SEQ ID NO. 108113 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 63544, or a homolog or fragment thereof, or a homolog thereof being depicted in Table I, application no. 6, column 8, in the same line as SEQ ID NO. 63544, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 63544 is depicted, plastidic is mentioned, or a mitochondrial transit peptide in case in Table I, application no. 6, in column 6 in the same line as SEQ ID NO. 63544 is depicted, mitochondrial is mentioned; which is operable linked, too.

In another embodiment thereof the expression cassette does not comprise an additional transit peptide in case in Table I, application no. 6, in column 6 in the same line as SEQ ID NO. 63544 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 90103, or a homolog or fragment thereof, or a homolog thereof being depicted in Table I, application no. 6, column 8, in the same line as SEQ ID NO. 90103, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 90103 is depicted, plastidic is mentioned, or a mitochondrial transit peptide in case in Table I, application no. 6, in column 6 in the same line as SEQ ID NO. 90103 is depicted, mitochondrial is mentioned; which is operable linked, too.

In another embodiment thereof the expression cassette does not comprise an additional transit peptide in case in Table I, application no. 6, in column 6 in the same line as SEQ ID NO. 90103 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 97298, or a homolog or fragment thereof, or a homolog thereof being depicted in Table I, application no. 6, column 8, in the same line as SEQ ID NO. 97298, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 97298 is depicted, plastidic is mentioned, or a mitochondrial transit peptide in case in Table I, application no. 6, in column 6 in the same line as SEQ ID NO. 97298 is depicted, mitochondrial is mentioned; which is operable linked, too.

In another embodiment thereof the expression cassette does not comprise an additional transit peptide in case in Table I, application no. 6, in column 6 in the same line as SEQ ID NO. 97298 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 64177, or a homolog or fragment thereof, or a homolog thereof being depicted in Table I, application no. 6, column 8, in the same line as SEQ ID NO. 64177, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 64177 is depicted, plastidic is mentioned, or a mitochondrial transit peptide in case in Table I, application no. 6, in column 6 in the same line as SEQ ID NO. 64177 is depicted, mitochondrial is mentioned; which is operable linked, too.

In another embodiment thereof the expression cassette does not comprise an additional transit peptide in case in Table I, application no. 6, in column 6 in the same line as SEQ ID NO. 64177 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 108487, or a homolog or fragment thereof, or a homolog thereof being depicted in Table I, application no. 6, column 8, in the same line as SEQ ID NO. 108487, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 108487 is depicted, plastidic is mentioned, or a mitochondrial transit peptide in case in Table I, application no. 6, in column 6 in the same line as SEQ ID NO. 108487 is depicted, mitochondrial is mentioned; which is operable linked, too.

In another embodiment thereof the expression cassette does not comprise an additional transit peptide in case in Table I, application no. 6, in column 6 in the same line as SEQ ID NO. 108487 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 90284, or a homolog or fragment thereof, or a homolog thereof being depicted in Table I, application no. 6, column 8, in the same line as SEQ ID NO. 90284, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 90284 is depicted, plastidic is mentioned, or a mitochondrial transit peptide in case in Table I, application no. 6, in column 6 in the same line as SEQ ID NO. 90284 is depicted, mitochondrial is mentioned; which is operable linked, too.

In another embodiment thereof the expression cassette does not comprise an additional transit peptide in case in Table I, application no. 6, in column 6 in the same line as SEQ ID NO. 90284 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 64470, or a homolog or fragment thereof, or a homolog thereof being depicted in Table I, application no. 6, column 8, in the same line as SEQ ID NO. 64470, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 64470 is depicted, plastidic is mentioned, or a mitochondrial transit peptide in case in Table I, application no. 6, in column 6 in the same line as SEQ ID NO. 64470 is depicted, mitochondrial is mentioned; which is operable linked, too.

In another embodiment thereof the expression cassette does not comprise an additional transit peptide in case in Table I, application no. 6, in column 6 in the same line as SEQ ID NO. 64470 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 108514, or a homolog or fragment thereof, or a homolog thereof being depicted in Table I, application no. 6, column 8, in the same line as SEQ ID NO. 108514, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 108514 is depicted, plastidic is mentioned, or a mitochondrial transit peptide in case in Table I, application no. 6, in column 6 in the same line as SEQ ID NO. 108514 is depicted, mitochondrial is mentioned; which is operable linked, too.

In another embodiment thereof the expression cassette does not comprise an additional transit peptide in case in Table I, application no. 6, in column 6 in the same line as SEQ ID NO. 108514 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 14885, or a homolog or fragment thereof, or a homolog thereof being depicted in Table I, application no. 6, column 8, in the same line as SEQ ID NO. 14885, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 14885 is depicted, plastidic is mentioned, or a mitochondrial transit peptide in case in Table I, application no. 6, in column 6 in the same line as SEQ ID NO. 14885 is depicted, mitochondrial is mentioned; which is operable linked, too.

In another embodiment thereof the expression cassette does not comprise an additional transit peptide in case in Table I, application no. 6, in column 6 in the same line as SEQ ID NO. 14885 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 90409, or a homolog or fragment thereof, or a homolog thereof being depicted in Table I, application no. 6, column 8, in the same line as SEQ ID NO. 90409, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 90409 is depicted, plastidic is mentioned, or a mitochondrial transit peptide in case in Table I, application no. 6, in column 6 in the same line as SEQ ID NO. 90409 is depicted, mitochondrial is mentioned; which is operable linked, too.

In another embodiment thereof the expression cassette does not comprise an additional transit peptide in case in Table I, application no. 6, in column 6 in the same line as SEQ ID NO. 90409 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 66274, or a homolog or fragment thereof, or a homolog thereof being depicted in Table I, application no. 6, column 8, in the same line as SEQ ID NO. 66274, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 66274 is depicted, plastidic is mentioned, or a mitochondrial transit peptide in case in Table I, application no. 6, in column 6 in the same line as SEQ ID NO. 66274 is depicted, mitochondrial is mentioned; which is operable linked, too.

In another embodiment thereof the expression cassette does not comprise an additional transit peptide in case in Table I, application no. 6, in column 6 in the same line as SEQ ID NO. 66274 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 90602, or a homolog or fragment thereof, or a homolog thereof being depicted in Table I, application no. 6, column 8, in the same line as SEQ ID NO. 90602, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 90602 is depicted, plastidic is mentioned, or a mitochondrial transit peptide in case in Table I, application no. 6, in column 6 in the same line as SEQ ID NO. 90602 is depicted, mitochondrial is mentioned; which is operable linked, too.

In another embodiment thereof the expression cassette does not comprise an additional transit peptide in case in Table I, application no. 6, in column 6 in the same line as SEQ ID NO. 90602 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 108723, or a homolog or fragment thereof, or a homolog thereof being depicted in Table I, application no. 6, column 8, in the same line as SEQ ID NO. 108723, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 108723 is depicted, plastidic is mentioned, or a mitochondrial transit peptide in case in Table I, application no. 6, in column 6 in the same line as SEQ ID NO. 108723 is depicted, mitochondrial is mentioned; which is operable linked, too.

In another embodiment thereof the expression cassette does not comprise an additional transit peptide in case in Table I, application no. 6, in column 6 in the same line as SEQ ID NO. 108723 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 98243, or a homolog or fragment thereof, or a homolog thereof being depicted in Table I, application no. 6, column 8, in the same line as SEQ ID NO. 98243, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 98243 is depicted, plastidic is mentioned, or a mitochondrial transit peptide in case in Table I, application no. 6, in column 6 in the same line as SEQ ID NO. 98243 is depicted, mitochondrial is mentioned; which is operable linked, too.

In another embodiment thereof the expression cassette does not comprise an additional transit peptide in case in Table I, application no. 6, in column 6 in the same line as SEQ ID NO. 98243 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 67190, or a homolog or fragment thereof, or a homolog thereof being depicted in Table I, application no. 6, column 8, in the same line as SEQ ID NO. 67190, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 67190 is depicted, plastidic is mentioned, or a mitochondrial transit peptide in case in Table I, application no. 6, in column 6 in the same line as SEQ ID NO. 67190 is depicted, mitochondrial is mentioned; which is operable linked, too.

In another embodiment thereof the expression cassette does not comprise an additional transit peptide in case in Table I, application no. 6, in column 6 in the same line as SEQ ID NO. 67190 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 91785, or a homolog or fragment thereof, or a homolog thereof being depicted in Table I, application no. 6, column 8, in the same line as SEQ ID NO. 91785, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 91785 is depicted, plastidic is mentioned, or a mitochondrial transit peptide in case in Table I, application no. 6, in column 6 in the same line as SEQ ID NO. 91785 is depicted, mitochondrial is mentioned; which is operable linked, too.

In another embodiment thereof the expression cassette does not comprise an additional transit peptide in case in Table I, application no. 6, in column 6 in the same line as SEQ ID NO. 91785 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 92668, or a homolog or fragment thereof, or a homolog thereof being depicted in Table I, application no. 6, column 8, in the same line as SEQ ID NO. 92668, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 92668 is depicted, plastidic is mentioned, or a mitochondrial transit peptide in case in Table I, application no. 6, in column 6 in the same line as SEQ ID NO. 92668 is depicted, mitochondrial is mentioned; which is operable linked, too.

In another embodiment thereof the expression cassette does not comprise an additional transit peptide in case in Table I, application no. 6, in column 6 in the same line as SEQ ID NO. 92668 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 98445, or a homolog or fragment thereof, or a homolog thereof being depicted in Table I, application no. 6, column 8, in the same line as SEQ ID NO. 98445, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 98445 is depicted, plastidic is mentioned, or a mitochondrial transit peptide in case in Table I, application no. 6, in column 6 in the same line as SEQ ID NO. 98445 is depicted, mitochondrial is mentioned; which is operable linked, too.

In another embodiment thereof the expression cassette does not comprise an additional transit peptide in case in Table I, application no. 6, in column 6 in the same line as SEQ ID NO. 98445 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68413, or a homolog or fragment thereof, or a homolog thereof being depicted in Table I, application no. 6, column 8, in the same line as SEQ ID NO. 68413, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 6, in column 6 in the same line as SEQ ID NO. 68413 is depicted, plastidic is mentioned, or a mitochondrial transit peptide in case in Table I, application no. 6, in column 6 in the same line as SEQ ID NO. 68413 is depicted, mitochondrial is mentioned; which is operable linked, too.

In another embodiment thereof the expression cassette does not comprise an additional transit peptide in case in Table I, application no. 6, in column 6 in the same line as SEQ ID NO. 68413 is depicted, non-targeted is mentioned.

[0165.1.1.6] to [0170.1.1.6] for the disclosure of these paragraphs see [0165.1.1.1] to [0170.1.1.1] above.

The invention further provides a vector, comprising a nucleic acid molecule comprising 1) a nucleic acid molecule selected from the group consisting of:
(a) a nucleic acid molecule encoding a polypeptide shown in Table II, column 5 or 8, application no. 6, preferably shown in Table II A, application no. 6, in column 5, or in Table II A, application no. 6, column 8, or in Table II B, application no. 6, column 8, or a homolog or a fragment thereof;
(b) a nucleic acid molecule shown in Table I, application no. 6, column 5 or 8, preferably shown in Table I A, application no. 6, in column 5, or in Table I A, application no. 6, column 8, or in Table I B, application no. 6, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 6, preferably shown in Table II A, application no. 6, in column 5, or in Table II A, application no. 6, column 8, or in Table II B, application no. 6, column 8;
(d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 6, preferably shown in Table I A, application no. 6, in column 5, or in Table I A, application no. 6, column 8, or in Table I B, application no. 6, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 6;
(i) a nucleic acid molecule encoding a polypeptide having the activity represented by a protein as depicted in column 5 of Table II, application no. 6;
(j) a nucleic acid molecule comprising a polynucleotide which is obtained by amplifying a cDNA library or a genomic library using the primers in Table III, application no. 6, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 6.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 6, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 6.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 6, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 6, in the same line as of the respective nucleic acid molecule according to 1) “plastidic” is depicted, or encoding a mitochondrial transit peptide, especially in case in column 6 of Table I, application no. 6, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 6, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 6, in the same line as of the respective nucleic acid molecule according to 1) “plastidic” is depicted, or encoding a mitochondrial transit peptide, especially in case in column 6 of Table I, application no. 6, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operable linked; a nucleic acid molecule encoding a transit peptide, especially in case in column 6 of Table I, application no. 6, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 6, in the same line as of the respective nucleic acid molecule according to 1) “plastidic” is depicted, or encoding a mitochondrial transit peptide, especially in case in column 6 of Table I, application no. 6, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 6 (in the same line).

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 6, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 6.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 6, in the same line as of the respective nucleic acid molecule according to 1) “plastidic” is depicted, or encoding a mitochondrial transit peptide, especially in case in column 6 of Table I, application no. 6, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 6, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 6, in the same line as of the respective nucleic acid molecule according to 1) “plastidic” is depicted, or encoding a mitochondrial transit peptide, especially in case in column 6 of Table I, application no. 6, in the same line as of the respective nu oleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 6 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 6, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 6 (in the same line) and confers a generation or an increase of the respective fine chemical in a non-human organism or a part thereof as compared to a corresponding wild-type non-human organism or part thereof.

In a preferred embodiment of said vector said nucleic acid molecule according to 1 (a), (b), (c), (d), (e), (f), (g), (h), (i), (j) or (k), said promoter and optionally said nucleic acid molecule encoding a transit peptide are encompassed in an expression cassette. Accordingly in an embodiment the invention provides a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

The invention further provides a vector comprising a nucleic acid molecule comprising 1) a nucleic acid molecule selected from the group consisting of:
(a) a nucleic acid molecule encoding a polypeptide shown in Table II, column 5 or 8, application no. 6, preferably shown in Table II A, application no. 6, in column 5, or in Table II A, application no. 6, column 8, or in Table II B, application no. 6, column 8, or a homolog or a fragment thereof;
(b) a nucleic acid molecule shown in Table I, application no. 6, column 5 or 8, preferably shown in Table I A, application no. 6, in column 5, or in Table I A, application no. 6, column 8, or in Table I B, application no. 6, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 6, preferably shown in Table II A, application no. 6, in column 5, or in Table II A, application no. 6, column 8, or in Table II B, application no. 6, column 8;
(d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 6, preferably shown in Table I A, application no. 6, in column 5, or in Table I A, application no. 6, column 8, or in Table I B, application no. 6, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 6;
(i) a nucleic acid molecule encoding a polypeptide having the activity represented by a protein as depicted in column 5 of Table II, application no. 6;
(j) a nucleic acid molecule comprising a polynucleotide which is obtained by amplifying a cDNA library or a genomic library using the primers in Table III, application no. 6, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto;
   whereby, preferably, the nucleic acid molecule according to 1 (a), (b), (c), (d), (e), (f), (g), (h), (i), (j) or (k) distinguishes over the sequence depicted in Table I A and/or I B, application no. 6, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 6, column 5 or 8, or the coding region thereof, respectively. In another embodiment, the nucleic acid molecule of the present invention, preferably the coding region thereof, is 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 6, column 5 or 8, or the coding region thereof, respectively. In a further embodiment the nucleic acid molecule does not encode the polypeptide sequence shown in Table II A and/or II B, application no. 6, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 6, column 5 or 8 and does not encode a protein of the sequence shown in Table II A and/or II B, application no. 6, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 6, column 5 and 8, or the coding region thereof. In a further embodiment, the protein of the present invention is at least 30%,40%, 50%, 60%, 70%, 80%, 90%, 95% identical to protein sequence depicted in Table II A and/or II B, application no. 6, column 5 or 8 but less than 100%, preferably less than 99.999%, 99.99% or 99.9%, more preferably less than 99%, 985, 97%, 96% or 95% identical to the sequence shown in Table II A and/or II B, application no. 6, column 5 or 8.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 6.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 6, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 6.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 6, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 6, in the same line as of the respective nucleic acid molecule according to 1) “plastidic” is depicted, or encoding a mitochondrial transit peptide, especially in case in column 6 of Table I, application no. 6, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 6, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 6, in the same line as of the respective nucleic acid molecule according to 1) “plastidic” is depicted, or encoding a mitochondrial transit peptide, especially in case in column 6 of Table I, application no. 6, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 6, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 6, in the same line as of the respective nucleic acid molecule according to 1) “plastidic” is depicted, or encoding a mitochondrial transit peptide, especially in case in column 6 of Table I, application no. 6, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 6.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 6, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 6.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 6, in the same line as of the respective nucleic acid molecule according to 1) “plastidic” is depicted, or encoding a mitochondrial transit peptide, especially in case in column 6 of Table I, application no. 6, in the same line as of the respective nu oleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 6, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 6, in the same line as of the respective nucleic acid molecule according to 1) “plastidic” is depicted, or encoding a mitochondrial transit peptide, especially in case in column 6 of Table I, application no. 6, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 6 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 6, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 6 and confers a generation or an increase of the respective fine chemical in a non-human organism or a part thereof as compared to a corresponding wild-type non-human organism or part thereof.

0181.1.1.6] to [0209.1.1.6] for the disclosure of these paragraphs see [0181.1.1.1] to [0209.1.1.1] above.

The genes of the invention, coding for an activity selected from the group consisting of 2,3-dihydroxyphenylpropionate 1,2-dioxygenase, 2-isopropylmalate synthase, 3-isopropylmalate dehydrogenase, 47266012-protein, 50S ribosomal protein L14, ABC transporter ATP binding component, ABC transporter ATP-binding protein, ABC transporter component, ABC transporter permease protein, acetolactate synthase, acetolactate synthase small subunit, acetyl CoA carboxylase, acetyl-CoA acetyltransferase, acetyltransferase, acid shock protein, acyl transferase, acyltransferase, adenylate kinase, adenylosuccinate lyase, agmatinase, aldehyde dehydrogenase, aminotransferase, anthranilate synthase component II, arginine exporter protein, At1g19800-protein, At1g23110-protein, At1g47380-protein, At4g32480-protein, At5g16650-protein, ATP-binding component of a transport system, auxin response factor, b0050-protein, b1003-protein, b1163-protein, b1445-protein, b1522-protein, b1670-protein, b1672-protein, b2513-protein, b2613-protein, b2673-protein, b2739-protein, b2812-protein, b3083-protein, b3490-protein, b3989-protein, b4029-protein, b4050-protein, b4056-protein, b4263-protein, beta-galactosidase, beta-hydroxylase, bifunctional protein (phosphoribosyltransferase and regulatory protein), binding-protein-dependent transport systems inner membrane component, branched chain aminotransferase, branched-chain amino acid permease, branched-chain amino-acid aminotransferase, calcium-dependent protein kinase, carbamoylphosphate synthase subunit, CBL-interacting protein kinase, cell division control protein, chaperone protein CIpB, cullin, cyclin, cyclin D, cytochrome c-type protein, dihydroxyacid dehydratase, disease resistance protein, DNA helicase II, DNA helicase IV, DNA mismatch repair protein, DNA polymerase, DNA-binding protein, elongation factor Tu, enoyl-CoA hydratase, eukaryotic translation initiation factor, eukaryotic translation initiation factor 5, ferredoxin, flavodoxin, fumarylacetoacetate hydrolase, GDP-mannose 4,6-dehydratase, geranylgeranyl pyrophosphate synthase, gluconate transport system permease 3, glucose-1-phosphate cytidylyltransferase, glucose-6-phosphate 1-dehydrogenase, glutamate-ammonia-ligase, glutamine synthetase, glutaredoxin, glycerol-3-phosphate dehydrogenase, glycogen (starch) synthase, glycogen synthase, glycoprotease, glycosyl transferase, GM02LC11114-protein, GM02LC5744-protein, H/ACA ribonucleoprotein complex subunit 3, harpin-induced family protein, heat shock transcription factor, hexose transporter, histone H2A, homocitrate synthase, homoserine dehydrogenase, hydrolase, iron(III) dicitrate-binding protein, isochorismate synthase, isopropylmalate isomerase large subunit, ketol-acid reductoisomerase, L-serine dehydratase, MADS-box transcription factor, major facilitator superfamily transporter protein, malate dehydrogenase, malic enzyme, membrane protein, membrane transport protein, methylglyoxal synthase, mitochondrial dicarboxylate transporter, monothiol glutaredoxin, murein transglycosylase, Na+/K+ transporter, NAD(P)H-quinone oxidoreductase subunit, NADH-quinone oxidoreductase subunit, NADP-dependent malic enzyme, Nucleoside monophosphate phosphohydrolase, O-antigen chain length determinant, oxidoreductase, oxireductase, peptidase, phosphate transporter, phosphoadenosine phosphosulfate reductase , phosphoanhydride phosphorylase, phosphoglycerate kinase, phosphopantetheine adenylyltransferase, photosystem II protein, polygalacturonase, polyphosphate kinase, precorrin methylase, protein kinase, protein phosphatase, protoporphyrin oxidase, purine nucleoside phosphorylase, purine-nucleoside phosphorylase, pyruvate kinase, riboflavin biosynthesis protein, ribosephosphate isomerase, constitutive, RNA binding protein, RNA-binding protein, Sec-independent protein translocase subunit, serine hydroxymethyltransferase, short-chain alcohol dehydrogenase family, sll1032-protein, sll1455-protein, sll1761-protein, superoxide dismutase, thioredoxin, thioredoxin family protein, threonine dehydratase, threonine synthase, transcription factor, transcriptional regulator, transport protein, trehalose-phosphatase, TTC1386-protein, urease subunit, vacuolar ATPase subunit, valine-pyruvate transaminase, YAL013W-protein, ydr183w-protein, ygl237c-protein, YGR193C-protein, yhr006w-protein, YKL038W-protein, YLR224W-protein, zinc finger protein, zinc transporter, and Zm_{—}4842_BE510522-protein are also called “FCRP genes”.

8 0211.1.1.6] to [0225.1.1.6] for the disclosure of these paragraphs see [0211.1.1.1] to [0225.1.1.1] above.

In addition to the sequence mentioned in Table I, application no. 6, column 5 or 8, preferably the coding region thereof, or its homologs or fragments, it may be advantageous additionally to express and/or mutate further genes in the non-human organisms. Especially advantageously, additionally at least one further gene of the biosynthetic pathway of the fine chemical is expressed in the non-human organisms such as plants or microorganisms. It is also possible that the regulation of the natural genes has been modified advantageously so that the gene and/or its gene product is no longer subject to the regulatory mechanisms which exist in the non-human organisms. This leads to an increased synthesis of the respective amino acid since, for example, feedback regulations no longer exist to the same extent or not at all. In addition it might be advantageously to combine the nucleic acids sequences of the invention containing the sequences shown in the respective line in Table I, application no. 6, column 5 or 8, preferably the coding region thereof, or homologs or fragments thereof, with genes which generally support or enhance the growth or yield of the target non-human organisms, for example genes which lead to faster growth rate of non human organism like microorganisms or plants or genes which produce stress-, pathogen-, or herbicide-resistant plants.

[0227.1.1.6] to [0239.1.1.6] for the disclosure of these paragraphs see [0227.1.1.1] to [0239.1.1.1] above.

In an embodiment of the present invention the nucleic acid construct of the present invention comprises a nucleic acid molecule as depicted in the respective line in Table I, application no. 6, column 5 or 8, preferably the coding region thereof, a fragment or a homolog thereof, and a plant promoter, such as the Big 35S promoter, the DC3 promoter, the PC Ubi promoter, the LegB4-promoter, the Super promoter, the USP promoter or the like. In another embodiment the nucleic acid construct of the present invention comprises a nucleic acid molecule as depicted in the respective line in Table I, application no. 6, column 5 or 8, preferably the coding region thereof, a fragment or a homolog thereof, and a plant promoter such as, for example, the USP, the LegB4-, the DC3 promoter or the ubiquitin promoter from parsley or other herein mentioned promoters. In a further embodiment the nucleic acid construct of the present invention comprises a nucleic acid molecule as depicted in the respective line in Table I, application no. 6, column 5 or 8, preferably the coding region thereof, a fragment or a homolog thereof, and a plant promoter, such as the Big 35S promoter, the PC Ubi promoter, the Super promoter, the USP promoter or the like. In addition different terminators may advantageously be used in these nucleic acid constructs; examples for transcriptional termination are polyadenylation signals.

[0241.1.1.6] to [0245.1.1.6] for the disclosure of these paragraphs see [0241.1.1.1] to [0245.1.1.1] above.

Other preferred sequences for use in operable linkage in gene expression constructs are targeting sequences, which are required for targeting the gene product into specific cell compartments (for a review, see Kermode, Crit. Rev. Plant Sci. 15 (4), 285 (1996) and references cited therein), for example into the vacuole, the nucleus, all types of plastids, such as amyloplasts, chloroplasts, chromoplasts, the extracellular space, the mitochondria, the endoplasmic reticulum, elaioplasts, peroxisomes, glycosomes, and other compartments of cells or extracellular; preferred are sequences, which are involved in targeting to plastids as mentioned above. Sequences, which must be mentioned in this context are, in particular, the signal-peptide- or transit-peptide-encoding sequences which are known per se. For example, plastid-transit-peptide-encoding sequences enable the targeting of the expression product into the plastids of a plant cell. Targeting sequences are also known for eukaryotic and to a lower extent for prokaryotic organisms and can advantageously be operable linked with the nucleic acid molecule of the present invention as shown in Table I, application no. 6, columns 5 and 8, preferably the coding region thereof, or homologs or fragments thereof, and described herein to achieve an expression in one of said compartments or extracellular.

[0247.1.1.6] to [0266.1.1.6] for the disclosure of these paragraphs see [0247.1.1.1] to [0266.1.1.1] above.

An embodiment of the invention relates to a vector where the nucleic acid molecule according to the invention is linked operably to regulatory sequences which permit the expression in a prokaryotic or eukaryotic host. A further embodiment of the invention relates to a vector in which a nucleic acid sequence encoding one of the polypeptides shown in the respective line in Table II, application no. 6, columns 5 or 8, or homologs or fragments thereof, is functionally linked to a targeting sequence such as a plastidial or mitochondrial targeting sequence. A further embodiment of the invention relates to a vector in which a nucleic acid sequence encoding one of the polypeptides shown in the respective line in Table II, application no. 6, columns 5 or 8, or homologs or fragments thereof, is functionally linked to (a) regulatory sequence which permit the expression in an organelle, such as plastids or mitochondria, preferably plastids.

A further embodiment of the invention relates to a vector which comprises a nucleic acid sequence encoding one of the polypeptides shown in the respective line in Table II, application no. 6, columns 5 or 8, or homologs or fragments thereof, but no additional targeting sequence.

[0268.1.1.6] to [0273.1.1.6] for the disclosure of these paragraphs see [0268.1.1.1] to [0273.1.1.1] above.

A further object of the invention relates to the use of a nucleic acid construct, e.g. an expression cassette, comprising one or more nucleic acid molecules encoding one or more polypeptides as depicted in the respective line(s) in Table II, application no. 6, columns 5 or 8, or homologs or fragments thereof, or comprising one or more nucleic acid molecules as depicted in the respective line(s) in Table I, application no. 6, columns 5 or 8, preferably the coding region thereof, or homologs or fragments thereof, or comprising a nucleic acid molecule hybridizing therewith for the transformation of a microorganism or a plant cell, plant tissues or a part of a plant

In doing so, depending on the choice of promoter, the nucleic acid molecules shown in the respective line in Table I, application no. 6, column 5 or 8, preferably the coding region thereof, or homologs or fragments thereof, or the nucleic acid molecules encoding a polypeptide as depicted in the respective line in Table II, application no. 6, column 5 or 8, or homologs or fragments thereof, can be expressed in the whole plant or specifically e.g. in the leaves, in the seeds, the nodules, in roots, in the stem or other parts of the plant. Those transgenic plants overproducing sequences, e.g. as depicted in the respective line in Table I, application no. 6, column 5 or 8, preferably the coding region thereof, or homologs or fragments thereof, the plant cell, plant tissues, plants, the reproductive material thereof, or parts thereof are a further object of the present invention.

Within the framework of the present invention, the production of the isoleucine, leucine, or valine is due to the generation or over-expression of one or more polypeptides as depicted in the respective line(s) in Table II, application no. 6, column 5 or 8, or homologs or fragments thereof, or encoded by the corresponding nucleic acid molecules as depicted in the respective line(s) in Table I, application no. 6, column 5 or 8, preferably the coding region thereof, or homologs or fragments thereof, in the non-human organism according to the invention, advantageously in the transgenic microorganism or plant according to the invention, at least for the duration of at least one plant generation.

A constitutive expression of the polypeptide as depicted in the respective line in Table II, application no. 6, column 5 or 8, or homologs or fragments thereof, or encoded by the respective nucleic acid molecule as depicted in the respective line in Table I, application no. 6, column 5 or 8, preferably the coding region thereof, or homologs or fragments thereof, may be advantageous. On the other hand, an inducible expression may also appear desirable. Expression of the polypeptide sequences of the invention can be either directed to the cytosol or to the organelles, such as plastids or mitochondria, preferably the plastids of the host cells, preferably the plant cells, or non-targeted.

for the disclosure of this paragraph see [0278.1.1.1] above.

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 6, column 5 or 8, preferably shown in Table II A, application no. 6, column 5, or in Table II A, application no. 6, column 8, or in Table II B, application no. 6, column 8, or a homolog or a fragment thereof;
   (b) a nucleic acid molecule shown in Table I, application no. 6, column 5 or 8, preferably shown in Table I A, application no. 6, column 5, or in Table I A, application no. 6, column 8, or in Table I B, application no. 6, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 6, preferably shown in Table II A, application no. 6, column 5, or in Table II A, application no. 6, column 8, or in Table II B, application no. 6, column 8;
   (d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 6, preferably shown in Table I A, application no. 6, column 5, or in Table I A, application no. 6, column 8, or in Table I B, application no. 6, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 6;
   (i) a nucleic acid molecule encoding a polypeptide having the activity represented by a respective protein as depicted in column 5 of Table II, application no. 6;
   (j) a nucleic acid molecule comprising a polynucleotide which is obtained by amplifying a cDNA library or a genomic library using the primers in Table III, application no. 6, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising an expression cassette comprising
[0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 6, column 5 or 8, preferably shown in Table II A, application no. 6, column 5, or in Table II A, application no. 6, column 8, or in Table II B, application no. 6, column 8, or a homolog or a fragment thereof;
   (b) a nucleic acid molecule shown in Table I, application no. 6, column 5 or 8, preferably shown in Table I A, application no. 6, column 5, or in Table I A, application no. 6, column 8, or in Table I B, application no. 6, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 6, preferably shown in Table II A, application no. 6, column 5, or in Table II A, application no. 6, column 8, or in Table II B, application no. 6, column 8;
   (d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 6, preferably shown in Table I A, application no. 6, column 5, or in Table I A, application no. 6, column 8, or in Table I B, application no. 6, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 6;
   (i) a nucleic acid molecule encoding a polypeptide having the activity represented by a respective protein as depicted in column 5 of Table II, application no. 6;
   (j) a nucleic acid molecule comprising a polynucleotide which is obtained by amplifying a cDNA library or a genomic library using the primers in Table III, application no. 6, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising or being transformed by a vector comprising
[1) a nucleic acid molecule selected from the group consisting of
   (a) a nucleic acid molecule encoding a polypeptide shown in Table II, application no. 6, column 5 or 8, preferably shown in Table II A, application no. 6, column 5, or in Table II A, application no. 6, column 8, or in Table II B, application no. 6, column 8, or a homolog or a fragment thereof;
   (b) a nucleic acid molecule shown in Table I, application no. 6, column 5 or 8, preferably shown in Table I A, application no. 6, column 5, or in Table I A, application no. 6, column 8, or in Table I B, application no. 6, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 6, preferably shown in Table II A, application no. 6, column 5, or in Table II A, application no. 6, column 8, or in Table II B, application no. 6, column 8;
   (d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 6, preferably shown in Table I A, application no. 6, column 5, or in Table I A, application no. 6, column 8, or in Table I B, application no. 6, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 6;
   (i) a nucleic acid molecule encoding a polypeptide having the activity represented by a respective protein as depicted in column 5 of Table II, application no. 6;
   (j) a nucleic acid molecule comprising a polynucleotide which is obtained by amplifying a cDNA library or a genomic library using the primers in Table III, application no. 6, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 6.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 6 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 6, in the same line as of the respective nucleic acid molecule according to 1) “plastidic” is depicted, or encoding a mitochondrial transit peptide, especially in case in column 6 of Table I, application no. 6, in the same line as of the respective nucleic acid molecule according to 1) “mitochondrial” is depicted.

In another embodiment of said transgenic non-human organism the expression cassette does not comprise 2) a nucleic acid molecule encoding a transit peptide, especially in case in column 6 of Table I, application no. 6, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 6.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 6, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 6, in the same line as of the respective nucleic acid molecule according to 1) “plastidic” is depicted, or encoding a mitochondrial transit peptide, especially in case in column 6 of Table I, application no. 6, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 6, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 6, in the same line as of the respective nucleic acid molecule according to 1) “plastidic” is depicted, or encoding a mitochondrial transit peptide, especially in case in column 6 of Table I, application no. 6, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 6, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 6, in the same line as of the respective nucleic acid molecule according to 1) “plastidic” is depicted, or encoding a mitochondrial transit peptide, especially in case in column 6 of Table I, application no. 6, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 6.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 6, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 6.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 6, in the same line as of the respective nucleic acid molecule according to 1) “plastidic” is depicted, or encoding a mitochondrial transit peptide, especially in case in column 6 of Table I, application no. 6, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 6, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 6, in the same line as of the respective nucleic acid molecule according to 1) “plastidic” is depicted, or encoding a mitochondrial transit peptide, especially in case in column 6 of Table I, application no. 6, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 6 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 6, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 6 (in the same line) and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 6, column 5 or 8, preferably shown in Table II A, application no. 6, column 5, or in Table II A, application no. 6, column 8, or in Table II B, application no. 6, column 8, or a homolog or a fragment thereof;
   (b) a nucleic acid molecule shown in Table I, application no. 6, column 5 or 8, preferably shown in Table I A, application no. 6, column 5, or in Table I A, application no. 6, column 8, or in Table I B, application no. 6, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 6, preferably shown in Table II A, application no. 6, column 5, or in Table II A, application no. 6, column 8, or in Table II B, application no. 6, column 8;
   (d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 6, preferably shown in Table I A, application no. 6, column 5, or in Table I A, application no. 6, column 8, or in Table I B, application no. 6, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 6;
   (i) a nucleic acid molecule encoding a polypeptide having the activity represented by a respective protein as depicted in column 5 of Table II, application no. 6;
   (j) a nucleic acid molecule comprising a polynucleotide which is obtained by amplifying a cDNA library or a genomic library using the primers in Table III, application no. 6, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
      or comprising an expression cassette comprising
[0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 6, column 5 or 8, preferably shown in Table II A, application no. 6, column 5, or in Table II A, application no. 6, column 8, or in Table II B, application no. 6, column 8, or a homolog or a fragment thereof;
   (b) a nucleic acid molecule shown in Table I, application no. 6, column 5 or 8, preferably shown in Table I A, application no. 6, column 5, or in Table I A, application no. 6, column 8, or in Table I B, application no. 6, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 6, preferably shown in Table II A, application no. 6, column 5, or in Table II A, application no. 6, column 8, or in Table II B, application no. 6, column 8;
   (d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 6, preferably shown in Table I A, application no. 6, column 5, or in Table I A, application no. 6, column 8, or in Table I B, application no. 6, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 6;
   (i) a nucleic acid molecule encoding a polypeptide having the activity represented by a respective protein as depicted in column 5 of Table II, application no. 6;
   (j) a nucleic acid molecule comprising a polynucleotide which is obtained by amplifying a cDNA library or a genomic library using the primers in Table III, application no. 6, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      and, if desired
2) nucleic acid molecule encoding a transit peptide, preferably encoding a plastidal transit peptide or a mitochondrial transit peptide;
   which are operable linked;]
      or comprising or being transformed by a vector comprising
[1) a nucleic acid molecule selected from the group consisting of
   (a) a nucleic acid molecule encoding a polypeptide shown in Table II, application no. 6, column 5 or 8, preferably shown in Table II A, application no. 6, column 5, or in Table II A, application no. 6, column 8, or in Table II B, application no. 6, column 8, or a homolog or a fragment thereof;
   (b) a nucleic acid molecule shown in Table I, application no. 6, column 5 or 8, preferably shown in Table I A, application no. 6, column 5, or in Table I A, application no. 6, column 8, or in Table I B, application no. 6, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 6, preferably shown in Table II A, application no. 6, column 5, or in Table II A, application no. 6, column 8, or in Table II B, application no. 6, column 8;
   (d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 6, preferably shown in Table I A, application no. 6, column 5, or in Table I A, application no. 6, column 8, or in Table I B, application no. 6, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 6;
   (i) a nucleic acid molecule encoding a polypeptide having the activity represented by a respective protein as depicted in column 5 of Table II, application no. 6;
   (j) a nucleic acid molecule comprising a polynucleotide which is obtained by amplifying a cDNA library or a genomic library using the primers in Table III, application no. 6, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      whereby, preferably, the nucleic acid molecule according to 1 (a), (b), (c), (d), (e), (f), (g), (h), (i), (j) or (k) distinguishes over the sequence depicted in Table I A and/or I B, application no. 6, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 6, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 6, column 5 or 8, or the coding region thereof, respectively. In a further embodiment the nucleic acid molecule does not encode the polypeptide sequence shown in Table II A and/or II B, application no. 6, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 6, column 5 or 8 and does not encode a protein of the sequence shown in Table II A and/or II B, application no. 6, column 5 or 8. Accordingly, in one embodiment, the protein encoded by a sequence of a nucleic acid according to 1 (a), (b), (c), (d), (e), (f), (g), (h), (i), (j) or (k) does not consist of the sequence shown in Table I A and/or I B, application no. 6, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 6, column 5 or 8 but less than 100%, preferably less than 99.999%, 99.99%, 99.9%, 99%, 98%, 97%, 96% or 95% identical to the sequence shown in Table II A and/or II B, application no. 6, column 5 or 8;
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a poly peptide which has the activity of the respective polypeptide represented by a protein comprising a polypeptide as depicted in the respective line in column 5 of Table II, application no. 6.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 6 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 6, in the same line as of the respective nucleic acid molecule according to 1) “plastidic” is depicted, or encoding a mitochondrial transit peptide, especially in case in column 6 of Table I, application no. 6, in the same line as of the respective nucleic acid molecule according to 1) “mitochondrial” is depicted.

In another embodiment thereof the expression cassette does not comprise 2) a nucleic acid molecule encoding a transit peptide, especially in case in column 6 of Table I, application no. 6, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 6.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the same line in column 5 of Table II, application no. 6, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 6, in the same line as of the respective nucleic acid molecule according to 1) “plastidic” is depicted, or encoding a mitochondrial transit peptide, especially in case in column 6 of Table I, application no. 6, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 6, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 6, in the same line as of the respective nucleic acid molecule according to 1) “plastidic” is depicted, or encoding a mitochondrial transit peptide, especially in case in column 6 of Table I, application no. 6, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 6, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 6, in the same line as of the respective nucleic acid molecule according to 1) “plastidic” is depicted, or encoding a mitochondrial transit peptide, especially in case in column 6 of Table I, application no. 6, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 6.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 6, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 6.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 6, in the same line as of the respective nucleic acid molecule according to 1) “plastidic” is depicted, or encoding a mitochondrial transit peptide, especially in case in column 6 of Table I, application no. 6, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 6, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 6, in the same line as of the respective nucleic acid molecule according to 1) “plastidic” is depicted, or encoding a mitochondrial transit peptide, especially in case in column 6 of Table I, application no. 6, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 6 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 6, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 6 and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

[0291.1.1.6] to [0299.1.1.6] for the disclosure of these paragraphs see [0291.1.1.1] to [0299.1.1.1] above.

The term “biological active portion” or “biological activity” means a polypeptide as depicted in the respective line in Table II, application no. 6, column 3 or a portion of said polypeptide which still has at least 10% or 20%, preferably 30%, 40%, 50% or 60%, especially preferably 70%, 75%, 80%, 90% or 95% of the enzymatic or biological activity of the natural or starting enzyme or protein.

[0301.1.1.6] to [0304.1.1.6] for the disclosure of these paragraphs see [0301.1.1.1] to 0304.1.1.1] above.

Synthetic oligonucleotide primers for the amplification, e.g. as shown in the respective line in Table III, application no. 6, column 8, by means of polymerase chain reaction can be generated on the basis of a sequence shown herein, for example the sequence shown in the respective line in Table I, application no. 6, columns 5 or 8, or the sequences derived from Table II, application no. 6, columns 5 or 8, respectively.

Moreover, it is possible to identify a conserved region of a protein from various organism by carrying out protein sequence alignments with the polypeptide encoded by the nucleic acid molecules of the present invention, in particular with the sequences encoded by the nucleic acid molecule shown in the respective line in column 5 or 8 of Table I, application no. 6, from which conserved regions, and in turn, degenerate primers can be derived. Conserved regions are those, which show a very little variation in the amino acid in one particular position of several homologs from different origin. The consensus sequence and polypeptide motifs shown in the respective line in column 8 of Table IV, application no. 6, are derived from such alignments of homologous proteins.

In an embodiment of the present invention, in the method of the present invention the activity of a polypeptide comprising or consisting of a consensus sequence or at least one polypeptide motif shown in the respective line in Table IV, application no. 6, column 8, is increased.

In another embodiment thereof, the method of the present invention relates to a polypeptide comprising or consisting of a consensus sequence shown in the respective line in Table IV, application no. 6, column 8 or comprising at least one polypeptide motif shown in the respective line in Table IV, application no. 6, column 8, whereby not more than 20, 15, 10, 9, 8, 7, 6, 5 4, 3, 2 or 1, or 0 of the amino acid positions indicated can be replaced by any amino acid.

In another embodiment thereof, the method of the present invention relates to a polypeptide comprising or consisting of a consensus sequence shown in the respective line in Table IV, application no. 6, column 8 or comprising at least one polypeptide motif shown in the respective line in Table IV, application no. 6, column 8, whereby not more than 15%, 10%, 5%, 4%, 3%, 2% or 1%, or 0% of the amino acid positions defined as distinct specific amino acids are/is replaced by another amino acid.

In another embodiment thereof, the method of the present invention relates to a polypeptide comprising or consisting of a consensus sequence shown in the respective line in Table IV, application no. 6, column 8 or comprising at least one polypeptide motif shown in the respective line in Table IV, application no. 6, column 8, whereby not more than 20, 15, 10, 9, 8, 7, 6, 5, 4, 3, 2 or 1, or 0 amino acids are inserted into a consensus sequence or polypeptide motif.

In an embodiment of the present invention, in the method of the present invention the activity of a polypeptide comprising or consisting of a consensus sequence or at least one polypeptide motif shown in the respective line in Table IV, application no. 6, column 8, is increased, whereby said polypeptide distinguishes over the sequence depicted in Table II, application no. 6, columns 5 or 8 by one or more amino acids.

In one embodiment, said polypeptide of the invention distinguishes over the sequence shown in the respective line in Table II, application no. 6, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II B, application no. 6, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in Table II, application no. 6, columns 5 or 8.

[309.1.1.6] to [0321.1.1.6] for the disclosure of these paragraphs see [0309.1.1.1] to [0321.1.1.1] above.

Polypeptides having above-mentioned activity, i.e. conferring the production or the increased production of the fine chemical isoleucine, leucine, or valine, respectively, as compared to a corresponding, e.g. non-transformed, wild type non-human organism, like a microorganism or a plant cell, plant or part thereof, can be encoded by other DNA sequences which hybridize to the sequences shown in the respective line in Table I, application no. 6, columns 5 and 8, preferably the coding region thereof, at least under relaxed hybridization conditions and which encode the expression of polypeptides conferring the production or the increased production of the respective fine chemical isoleucine, leucine, or valine, respectively, as compared to a corresponding, e.g. non-transformed, wild type non-human organism, like a microorganism or a plant cell, plant or part thereof.

[0323.1.1.6] to [0329.1.1.6] for the disclosure of these paragraphs see [0323.1.1.1] to [0329.1.1.1] above.

Further, the nucleic acid molecule of the invention comprises a nucleic acid molecule, which is a complement of one of the nucleotide sequences of above-mentioned nucleic acid molecules or a portion thereof. A nucleic acid molecule or its sequence which is complementary to one of the nucleotide molecules or sequences shown in the respective line in Table I, application no. 6, columns 5 or 8, preferably the coding region thereof, homologs or fragments thereof, is one which is sufficiently complementary to one of the nucleotide molecules or sequences shown in the respective line in Table I, application no. 6, columns 5 or 8, preferably the coding region thereof, homologs or fragments thereof, such that it can hybridize to one of the nucleotide sequences shown in Table I, application no. 6, columns 5 or 8, preferably the coding region thereof, homologs or fragments thereof, thereby forming a stable duplex. Preferably, the hybridization is performed under stringent hybridization conditions. However, a complement of one of the herein disclosed sequences is preferably a sequence complement thereto according to the base pairing of nucleic acid molecules well known to the skilled person. For example, the bases A and G undergo base pairing with the bases T and U or C, resp. and visa versa. Modifications of the bases can influence the base-pairing partner.

The nucleic acid molecule of the invention comprises a nucleotide sequence which is at least about 30%, 35%, 40%, 45%, 50%, 55%, 60%, 65%, 70%, 75%, 80%, 90%, 95%, 96%, 97%, 98%, 99%, 99.5% or more homologous to a nucleotide sequence shown in Table I, application no. 6, columns 5 or 8, preferably the coding region thereof, homologs or fragments thereof, and preferably has above-mentioned activity, in particular conferring the production or the increased production of the fine chemical isoleucine, leucine, or valine, respectively, after increasing the activity or an activity of a gene as shown in the respective line in Table I or of a gene product, e.g. as shown in the respective line in Table II, application no. 6, column 5 or 8, by for example in one embodiment expression either in the cytosol or in an organelle such as a plastid or mitochondria or both, preferably in a plastid, or in another embodiment non-targeted or targeted.

The nucleic acid molecule of the invention comprises a nucleotide sequence or molecule which hybridizes, preferably hybridizes under stringent conditions as defined herein, to one of the nucleic acid molecule shown in the respective line in Table I, preferably Table IB, application no. 6, columns 5 or 8, preferably the coding region thereof, fragments or homologs thereof, and encodes a protein having above-mentioned activity, e.g. conferring the production or the increased production of the respective fine chemical isoleucine, leucine, or valine as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted, and optionally, the activity thereof is selected from the group consisting of 2,3-dihydroxyphenylpropionate 1,2-dioxygenase, 2-isopropylmalate synthase, 3-isopropylmalate dehydrogenase, 47266012-protein, 50S ribosomal protein L14, ABC transporter ATP binding component, ABC transporter ATP-binding protein, ABC transporter component, ABC transporter permease protein, acetolactate synthase, acetolactate synthase small subunit, acetyl CoA carboxylase, acetyl-CoA acetyltransferase, acetyltransferase, acid shock protein, acyl transferase, acyltransferase, adenylate kinase, adenylosuccinate lyase, agmatinase, aldehyde dehydrogenase, aminotransferase, anthranilate synthase component II, arginine exporter protein, At1g19800-protein, At1g23110-protein, At1g47380-protein, At4g32480-protein, At5g16650-protein, ATP-binding component of a transport system, auxin response factor, b0050-protein, b1003-protein, b1163-protein, b1445-protein, b1522-protein, b1670-protein, b1672-protein, b2513-protein, b2613-protein, b2673-protein, b2739-protein, b2812-protein, b3083-protein, b3490-protein, b3989-protein, b4029-protein, b4050-protein, b4056-protein, b4263-protein, beta-galactosidase, beta-hydroxylase, bifunctional protein (phosphoribosyltransferase and regulatory protein), binding-protein-dependent transport systems inner membrane component, branched chain aminotransferase, branched-chain amino acid permease, branched-chain amino-acid aminotransferase, calcium-dependent protein kinase, carbamoyl-phosphate synthase subunit, CBL-interacting protein kinase, cell division control protein, chaperone protein CIpB, cullin, cyclin, cyclin D, cytochrome c-type protein, dihydroxyacid dehydratase, disease resistance protein, DNA helicase II, DNA helicase IV, DNA mismatch repair protein, DNA polymerase, DNA-binding protein, elongation factor Tu, enoyl-CoA hydratase, eukaryotic translation initiation factor, eukaryotic translation initiation factor 5, ferredoxin, flavodoxin, fumarylacetoacetate hydrolase, GDP-mannose 4,6-dehydratase, geranylgeranyl pyrophosphate synthase, gluconate transport system permease 3, glucose-1-phosphate cytidylyltransferase, glucose-6-phosphate 1-dehydrogenase, glutamate-ammonia-ligase, glutamine synthetase, glutaredoxin, glycerol-3-phosphate dehydrogenase, glycogen (starch) synthase, glycogen synthase, glycoprotease, glycosyl transferase, GM02LC11114-protein, GM02LC5744-protein, H/ACA ribonucleoprotein complex subunit 3, harpin-induced family protein, heat shock transcription factor, hexose transporter, histone H2A, homocitrate synthase, homoserine dehydrogenase, hydrolase, iron(III) dicitrate-binding protein, isochorismate synthase, isopropylmalate isomerase large subunit, ketol-acid reductoisomerase, L-serine dehydratase, MADS-box transcription factor, major facilitator superfamily transporter protein, malate dehydrogenase, malic enzyme, membrane protein, membrane transport protein, methylglyoxal synthase, mitochondrial dicarboxylate transporter, monothiol glutaredoxin, murein transglycosylase, Na+/K+ transporter, NAD(P)H-quinone oxidoreductase subunit, NADH-quinone oxidoreductase subunit, NADP-dependent malic enzyme, Nucleoside monophosphate phosphohydrolase, O-antigen chain length determinant, oxidoreductase, oxireductase, peptidase, phosphate transporter, phosphoadenosine phosphosulfate reductase , phosphoanhydride phosphorylase, phosphoglycerate kinase, phosphopantetheine adenylyltransferase, photosystem II protein, polygalacturonase, polyphosphate kinase, precorrin methylase, protein kinase, protein phosphatase, protoporphyrin oxidase, purine nucleoside phosphorylase, purinenucleoside phosphorylase, pyruvate kinase, riboflavin biosynthesis protein, ribosephosphate isomerase, constitutive, RNA binding protein, RNA-binding protein, Sec-independent protein translocase subunit, serine hydroxymethyltransferase, short-chain alcohol dehydrogenase family, sll1032-protein, sll1455-protein, sll1761-protein, superoxide dismutase, thioredoxin, thioredoxin family protein, threonine dehydratase, threonine synthase, transcription factor, transcriptional regulator, transport protein, trehalose-phosphatase, TTC1386-protein, urease subunit, vacuolar ATPase subunit, valine-pyruvate transaminase, YAL013W-protein, ydr183w-protein, ygl237c-protein, YGR193C-protein, yhr006w-protein, YKL038W-protein, YLR224W-protein, zinc finger protein, zinc transporter, and Zm_{—}4842_BE510522-protein, respectively.

Moreover, the nucleic acid molecule of the invention can comprise only a portion of the coding region of one of the nucleic acid molecules depicted in Table I, application no. 6, columns 5 or 8, for example a fragment which can be used as a probe or primer or a fragment encoding a biologically active portion of the polypeptide of the present invention or of a polypeptide used in the process of the present invention, i.e. having above-mentioned activity, e.g. conferring the production or the increased production of the respective fine chemical isoleucine, leucine, or valine as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted. The nucleotide sequences determined from the cloning of the present protein-according-to-the-invention -encoding gene allows for the generation of probes and primers designed for use in identifying and/or cloning its homologues from other cell types and organisms. The probe/primer typically comprises a substantially purified oligonucleotide. The oligonucleotide typically comprises a region of nucleotide sequence that hybridizes under stringent conditions to at least about 12, 15 preferably about 20 or 25, more preferably about 40, 50 or 75 consecutive nucleotides of a sense strand of one of the sequences set forth, e.g., in the respective line in Table I, application no. 6, column 5 or 8, preferably the coding region thereof, or a homolog or a fragment thereof, an anti-sense sequence of one of the sequences, e.g., set forth in the respective line in Table I, application no. 6, columns 5 or 8, preferably the coding region thereof, or a homolog or a fragment thereof or naturally occurring mutants thereof. Primers based on a nucleic acid molecule of invention can be used in PCR reactions to clone homologs of the polypeptide of the invention or of the polypeptide used in the process of the invention, e.g. as the primers described in the examples of the present invention, e.g. as shown in the examples. A PCR with the primers shown in the respective line in Table III, column 8 will result in a fragment of the gene product as shown in Table II, application no. 6, column 5.

Primer sets are interchangeable. The person skilled in the art knows how to design and combine said primers to result in the desired product, e.g. in a full length clone or a partial sequence. Probes based on the sequences of the nucleic acid molecule of the invention or used in the process of the present invention can be used to detect transcripts or genomic sequences encoding the same or homologous proteins. The probe can further comprise a label group attached thereto, e.g. the label group can be a radioisotope, a fluorescent compound, an enzyme, or an enzyme co-factor. Such probes can be used as a part of a genomic marker test kit for identifying cells which express a polypeptide of the invention or used in the process of the present invention, such as by measuring a level of an encoding nucleic acid molecule in a sample of cells, e.g. detecting mRNA levels or determining, whether a genomic gene comprising the sequence of the polynucleotide of the invention or used in the processes of the present invention has been mutated or deleted.

As the expression of a respective nucleic acids of the invention is related to the synthesis of the respective fine chemical isoleucine, leucine, or valine its function as a probe extends to the detection of microorganisms, plant tissues, plants, plant variets, plant ecotypes or plant genera with varying capability or potential for synthesis of the respective fine chemical isoleucine, leucine, or valine. Therefore in one embodiment the present invention relates to a method for analyzing the capability or potential of a plant tissue, a plant, a plant variety or plant ecotype to produce the fine chemical isoleucine, leucine, or valine by using the nucleic acid of the invention or parts thereof as a probe to detect the amount of the nucleic acid of the invention in the non-human organism or a part thereof in comparison to another non-human organism.

The nucleic acid molecule of the invention encodes a polypeptide or portion thereof which includes an amino acid sequence which is sufficiently homologous to the amino acid sequence shown in respective line in Table II, application no. 6, columns 5 or 8 such that the protein or portion thereof maintains the ability to confer the production or the increased production of the respective fine chemical isoleucine, leucine, or valine as compared to a corresponding, e.g. non-transformed, wild type non-human organism, like a microorganism or a plant cell, plant or part thereof, in particular increasing the activity as mentioned above or as described in the examples in microorganisms or plants.

As used herein, the language “sufficiently homologous” refers to proteins or portions thereof which have amino acid sequences which include a minimum number of identical or equivalent amino acids (e.g. an amino acid which has a similar side chain as an amino acid in one of the sequences of the polypeptide of the present invention) to an amino acid sequence shown in the respective line in Table II, application no. 6, columns 5 or 8 such that the protein or portion thereof is able to confer the production or the increased production of the respective fine chemical isoleucine, leucine, or valine as compared to a corresponding, e.g. non-transformed, wild type non-human organism, like a microorganism or a plant cell, plant or part thereof. For example having the activity of a protein as shown in the respective line in Table II, application no. 6, column 3 and as described herein.

In an embodiment of the present invention, the nucleic acid molecule of the present invention comprises a nucleic acid molecule that encodes a portion of the protein of the present invention. The protein is at least about 30%, 35%, 40%, 45% 50%, 55%, 60%, 65%, 70%, 75%, 80%, 85%, 90%, 91%, 92%, 93%, 94%, 95%, 96%, 97%, 98%, 99%, 99.5% or more homologous to an entire amino acid sequence shown in the respective line of Table II, application no. 6, column 5 or 8 and has the above-mentioned activity, e.g. conferring the production or the increased production of the respective fine chemical isoleucine, leucine, or valine as compared to a corresponding, e.g. non-transformed, wild type non-human organism, like a microorganism or a plant cell, plant or part thereof by, for example in a embodiment expression either in the cytosol or in an organelle such as a plastid or mitochondria or both, preferably in a plastid, or in another embodiment by targeted or non-targeted expression.

[0338.1.1.6] to [0339.1.1.6] for the disclosure of these paragraphs see [0338.1.1.1] to [0339.1.1.1] above.

The invention further relates to nucleic acid molecules or methods using said nucleic acid molecules that differ from one of the nucleotide sequences shown in the respective line in Table I, application no. 6, columns 5 or 8, preferably the coding region thereof, or fragments or homologs thereof, due to degeneracy of the genetic code but encode a polypeptide of the present invention, in particular a polypeptide having the above-mentioned activity, e.g. as those polypeptides depicted in the respective line in Table II, application no. 6, columns 5 or 8, fragments or homologs thereof. Advantageously, the nucleic acid molecule of the invention comprises, or in an other embodiment has, a nucleotide sequence encoding a protein comprising, or in an other embodiment having, an amino acid sequence shown in the respective line in Table II, application no. 6, columns 5 or 8, fragments or homologs thereof. In a further embodiment, the nucleic acid molecule of the invention encodes a full length protein which is substantially homologous to an amino acid sequence shown in the respective line in Table II, application no. 6, columns 5 or 8, or homologs thereof. However, in an embodiment, the nucleic acid molecule of the present invention does not consist of the sequence shown in the respective line in Table I, preferably Table IA, application no. 6, columns 5 or 8, preferably the coding region thereof, but the functional homologs thereof.

[0341.1.1.6] to [0343.1.1.6] for the disclosure of these paragraph see [0341.1.1.1] to [0343.1.1.1] above.

Accordingly, in another embodiment, a nucleic acid molecule of the invention is at least 15, 20, 25 or 30 nucleotides in length. Preferably, it hybridizes under stringent conditions to a nucleic acid molecule comprising a nucleotide sequence of the nucleic acid molecule of the present invention or used in the process of the present invention, e.g. comprising the sequence shown in the respective line in Table I, application no. 6, columns 5 or 8, preferably the coding region thereof. In another embodiment the nucleic acid molecule is preferably at least 20, 30, 50, 100, 250 or more nucleotides in length.

for the disclosure of this paragraph see [0345.1.1.1] above.

Preferably, a nucleic acid molecule of the invention that hybridizes under stringent conditions to a sequence shown in the respective line in Table I, application no. 6, columns 5 or 8, preferably the coding region thereof, corresponds to a naturally-occurring nucleic acid molecule of the invention. As used herein, a “naturally-occurring” nucleic acid molecule refers to a RNA or DNA molecule having a nucleotide sequence that occurs in nature (e.g. encodes a natural protein). Preferably, the nucleic acid molecule encodes a natural protein having above-mentioned activity, e.g. conferring the production or the increased production of the respective fine chemical isoleucine, leucine, or valine as compared to a corresponding, e.g. non-transformed, wild type non-human organism, like a microorganism or a plant cell, plant or part thereof after increasing the expression or activity thereof or the activity of a protein of the invention or used in the process of the invention, in an embodiment for example expression either in the cytosol or in an organelle such as a plastid or mitochondria, preferably in plastids, or, in another embodiment by targeted or non-targeted expression.

for the disclosure of this paragraph see [0347.1.1.1] above.

For example, nucleotide substitutions leading to amino acid substitutions at “non-essential” amino acid residues can be made in a sequence of the nucleic acid molecule of the invention or used in the process of the invention, e.g. shown in the respective line in Table I, application no. 6, columns 5 or 8, preferably the coding region thereof, or fragments or homologs thereof.

[0349.1.1.6] to [0350.1.1.6] for the disclosure of these paragraphs see [0349.1.1.1] to [0350.1.1.1] above.

Accordingly, the invention relates to nucleic acid molecules encoding a polypeptide having above-mentioned activity, in a non-human organisms or parts thereof by for example expression either in the cytosol or in an organelle such as a plastid or mitochondria or both, preferably in plastids, that contain changes in amino acid residues that are not essential for said activity. Such polypeptides differ in amino acid sequence from a sequence depicted in the respective line in Table II, especially Table IIA, application no. 6, columns 5 or 8, but retain said activity described herein. The nucleic acid molecule can comprise a nucleotide sequence encoding a polypeptide, wherein the polypeptide comprises an amino acid sequence at least about 50% identical to an amino acid sequence shown in the respective line in Table II, application no. 6, columns 5 or 8, and is capable of conferring the production or the increased production of the respective fine chemical isoleucine, leucine, or valine as compared to a corresponding, e.g. non-transformed, wild type non-human organism, like a microorganism or a plant cell, plant or part thereof, after increasing its activity for example in an embodiment by expression either in the cytosol or in an organelle such as a plastid or mitochondria or both, preferably in plastids, or, in another embodiment by targeted or non-targeted expression. Preferably, the protein encoded by the nucleic acid molecule is at least about 60%, 70%, 80%, 90%, 95%, 96%, 97%, 98%, 99% or 99.5% identical to the sequence shown in the respective line in Table II, application no. 6, columns 5 or 8.

[0352.1.1.6] to [0357.1.1.6] for the disclosure of these paragraphs see [0352.1.1.1] to [0357.1.1.1] above.

Functional equivalents of the nucleic acid molecules according to the present invention and/or used in the process according to the present invention represent an embodiment of homologs. Functional equivalents derived from the nucleic acid molecules as shown in the respective line in Table I, application no. 6, columns 5 or 8, preferably the coding region thereof, according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the nucleic acid molecules as shown in the respective line in Table I, application no. 6, columns 5 or 8, preferably the coding region thereof, according to the invention and encode polypeptides having essentially the same properties as the polypeptide as shown in the respective line in Table II, application no. 6, columns 5 or 8.

Functional equivalents derived from one of the polypeptides as shown in the respective line in Table II, application no. 6, columns 5 or 8 according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the polypeptides as shown in the respective line in Table II, application no. 6, columns 5 or 8 according to the invention and having essentially the same properties as the polypeptide as shown in the respective line in Table II, application no. 6, columns 5 or 8.

for the disclosure of this paragraph see [0359.1.1.1] above.

A nucleic acid molecule encoding a homolog to a polypeptide depicted in the respective line in Table II, application no. 6, columns 5 or 8 can be created by introducing one or more nucleotide substitutions, additions or deletions into the nucleic acid molecule of the present invention, in particular of Table I, application no. 6, columns 5 or 8, in the respective line, such that one or more amino acid substitutions, additions or deletions are introduced into the encoded protein. Mutations can be introduced into the nucleic acid molecules as depicted in the respective line in Table I, application no. 6, columns 5 or 8, preferably the coding region thereof, by standard techniques, such as site-directed mutagenesis, PCR-mediated mutagenesis or other methods known to the person skilled in the art.

[0361.1.1.6] to [0363.1.1.6] for the disclosure of these paragraphs see [0361.1.1.1] to [0363.1.1.1] above.

Homologues of the nucleic acid sequences used, with the sequences shown in the respective line in Table I, application no. 6, columns 5 or 8, preferably the coding region thereof, comprise also allelic variants with at least approximately 30%, 35%, 40%, 45%, 50%, 55%, 60%, 65%, 70%, 75%, 80%, 85%, 90%, 91%, 92%, 93%, 94% 95%. 96%, 97%, 98%, 99%, 99.5% or more % homology with one of the nucleotide sequences shown or the above-mentioned derived nucleic acid sequences or their homologues or derivatives or parts of these. Allelic variants encompass in particular functional variants which can be obtained by deletion, insertion or substitution of nucleotides from the sequences shown in the respective line, preferably in Table I, columns 5 or 8, preferably the coding region thereof, or from the derived nucleic acid sequences, the intention being, however, that the enzymatic activity or the biological activity of the resulting proteins synthesized is advantageously retained or increased.

In one embodiment of the present invention, the nucleic acid molecule of the invention or used in the process of the invention comprises the sequences shown in the respective line in any of the Table I, application no. 6, columns 5 or 8, preferably the coding region thereof, fragments or homologs thereof. It is preferred that the nucleic acid molecule comprises as little as possible other nucleotides not shown in the respective line in any one of Table I, application no. 6, columns 5 or 8, preferably the coding region thereof, fragments or homologs thereof. In one embodiment, the nucleic acid molecule comprises less than 500, 400, 300, 200, 100, 90, 80, 70, 60, 50 or 40 further nucleotides. In a further embodiment, the nucleic acid molecule comprises less than 30, 20 or 10 further nucleotides. In one embodiment, the nucleic acid molecule used in the process of the invention is identical to the sequences shown in the respective line in Table I, application no. 6, columns 5 or 8, preferably the coding region thereof.

Also preferred is that the nucleic acid molecule used in the process of the invention encodes a polypeptide comprising the sequence shown in the respective line in Table II, application no. 6, columns 5 or 8, fragments or homologs thereof. In one embodiment, the nucleic acid molecule encodes less than 150, 130, 100, 80, 60, 50, 40 or 30 further amino acids.

In a further embodiment, the encoded polypeptide comprises less than 20, 15, 10, 9, 8, 7, 6 or 5 further amino acids. In one embodiment used in the inventive process, the encoded polypeptide is identical to the sequences shown in the respective line in Table II, application no. 6, columns 5 or 8.

In one embodiment, the nucleic acid molecule of the invention or used in the process encodes a polypeptide comprising the sequence shown in the respective line in Table II, application no. 6, columns 5 or 8, or fragments or homologs thereof, and comprises less than 100 further nucleotides. In a further embodiment, said nucleic acid molecule comprises less than 30 further nucleotides. In one embodiment, the nucleic acid molecule used in the process is identical to a coding sequence of the sequences shown in the respective line in Table I, application no. 6, columns 5 or 8.

Polypeptides (=proteins), which still have the essential biological or enzymatic activity of the polypeptide of the present invention conferring the production or the increased production of the respective fine chemical isoleucine, leucine, or valine as compared to a corresponding, e.g. non-transformed, wild type non-human organism, like a microorganism or a plant cell, plant or part thereof, i.e. whose activity is essentially not reduced, are polypeptides with at least 10%, 20%, 30%, 40%, 50%, 60%, 70%, 80%, 90%, 95% or more of the wild type biological activity or enzymatic activity, advantageously, the activity is essentially not reduced in comparison with the activity of a polypeptide shown in the respective line in Table II, application no. 6, columns 5 or 8 expressed under identical conditions.

Homologues of nucleic acid molecules shown in the respective line in Table I, application no. 6, columns 5 or 8, preferably the coding region thereof, or fragments thereof, or of the derived sequences shown in the respective line in Table II, application no. 6, columns 5 or 8, or fragments thereof, also mean truncated sequences, cDNA, single-stranded DNA or RNA of the coding and noncoding DNA sequence. Homologues of said sequences are also understood as meaning derivatives, which comprise noncoding regions such as, for example, UTRs, introns, terminators, enhancers or promoter variants. The promoters upstream of the nucleotide sequences stated can be modified by one or more nucleotide substitution(s), insertion(s) and/or deletion(s) without, however, interfering with the functionality or activity either of the promoters, the open reading frame (=ORF) or with the 3′-regulatory region such as terminators or other 3′-regulatory regions, which are far away from the ORF. It is furthermore possible that the activity of the promoters is increased by modification of their sequence, or that they are replaced completely by more active promoters, even promoters from heterologous organisms. Appropriate promoters are known to the person skilled in the art and are mentioned herein below.

[0370.1.1.6] to [0379.1.1.6] for the disclosure of these paragraphs see [0370.1.1.1] to [0379.1.1.1] above.

Moreover, a native polypeptide conferring the increase of the respective fine chemical isoleucine, leucine, or valine in a non-human organism or a part thereof can be isolated from cells (e.g., endothelial cells), for example using the antibody of the present invention as described below, in particular, an antibody against proteins having 2,3-dihydroxyphenylpropionate 1,2-dioxygenase, 2-isopropylmalate synthase, 3-isopropylmalate dehydrogenase, 47266012-protein, 50S ribosomal protein L14, ABC transporter ATP binding component, ABC transporter ATP-binding protein, ABC transporter component, ABC transporter permease protein, acetolactate synthase, acetolactate synthase small subunit, acetyl CoA carboxylase, acetyl-CoA acetyltransferase, acetyltransferase, acid shock protein, acyl transferase, acyltransferase, adenylate kinase, adenylosuccinate lyase, agmatinase, aldehyde dehydrogenase, aminotransferase, anthranilate synthase component II, arginine exporter protein, At1g19800-protein, At1g23110-protein, At1g47380-protein, At4g32480-protein, At5g16650-protein, ATP-binding component of a transport system, auxin response factor, b0050-protein, b1003-protein, b1163-protein, b1445-protein, b1522-protein, b1670-protein, b1672-protein, b2513-protein, b2613-protein, b2673-protein, b2739-protein, b2812-protein, b3083-protein, b3490-protein, b3989-protein, b4029-protein, b4050-protein, b4056-protein, b4263-protein, beta-galactosidase, beta-hydroxylase, bifunctional protein (phosphoribosyltransferase and regulatory protein), binding-protein-dependent transport systems inner membrane component, branched chain aminotransferase, branched-chain amino acid permease, branched-chain amino-acid aminotransferase, calcium-dependent protein kinase, carbamoyl-phosphate synthase subunit, CBL-interacting protein kinase, cell division control protein, chaperone protein CIpB, cullin, cyclin, cyclin D, cytochrome c-type protein, dihydroxyacid dehydratase, disease resistance protein, DNA helicase II, DNA helicase IV, DNA mismatch repair protein, DNA polymerase, DNA-binding protein, elongation factor Tu, enoyl-CoA hydratase, eukaryotic translation initiation factor, eukaryotic translation initiation factor 5, ferredoxin, flavodoxin, fumarylacetoacetate hydrolase, GDP-mannose 4,6-dehydratase, geranylgeranyl pyrophosphate synthase, gluconate transport system permease 3, glucose-1-phosphate cytidylyltransferase, glucose-6-phosphate 1-dehydrogenase, glutamate-ammonia-ligase, glutamine synthetase, glutaredoxin, glycerol-3-phosphate dehydrogenase, glycogen (starch) synthase, glycogen synthase, glycoprotease, glycosyl transferase, GM02LC11114-protein, GM02LC5744-protein, H/ACA ribonucleoprotein complex subunit 3, harpin-induced family protein, heat shock transcription factor, hexose transporter, histone H2A, homocitrate synthase, homoserine dehydrogenase, hydrolase, iron(III) dicitrate-binding protein, isochorismate synthase, isopropylmalate isomerase large subunit, ketol-acid reductoisomerase, L-serine dehydratase, MADS-box transcription factor, major facilitator superfamily transporter protein, malate dehydrogenase, malic enzyme, membrane protein, membrane transport protein, methylglyoxal synthase, mitochondrial dicarboxylate transporter, monothiol glutaredoxin, murein transglycosylase, Na+/K+ transporter, NAD(P)H-quinone oxidoreductase subunit, NADH-quinone oxidoreductase subunit, NADP-dependent malic enzyme, Nucleoside monophosphate phosphohydrolase, O-antigen chain length determinant, oxidoreductase, oxireductase, peptidase, phosphate transporter, phosphoadenosine phosphosulfate reductase , phosphoanhydride phosphorylase, phosphoglycerate kinase, phosphopantetheine adenylyltransferase, photosystem II protein, polygalacturonase, polyphosphate kinase, precorrin methylase, protein kinase, protein phosphatase, protoporphyrin oxidase, purine nucleoside phosphorylase, purine-nucleoside phosphorylase, pyruvate kinase, riboflavin biosynthesis protein, ribosephosphate isomerase, constitutive, RNA binding protein, RNA-binding protein, Sec-independent protein translocase subunit, serine hydroxymethyltransferase, short-chain alcohol dehydrogenase family, sll1032-protein, sll1455-protein, sll1761-protein, superoxide dismutase, thioredoxin, thioredoxin family protein, threonine dehydratase, threonine synthase, transcription factor, transcriptional regulator, transport protein, trehalosephosphatase, TTC1386-protein, urease subunit, vacuolar ATPase subunit, valine-pyruvate transaminase, YAL013W-protein, ydr183w-protein, ygl237c-protein, YGR193C-protein, yhr006w-protein, YKL038W-protein, YLR224W-protein, zinc finger protein, zinc transporter, or Zm_{—}4842_BE510522-protein activity, respectively, or an antibody against polypeptides as shown in the respective line in Table II, application no. 6, columns 5 or 8, or fragments or homologs thereof which can be produced by standard techniques utilizing the polypeptid of the present invention or fragment thereof, i.e., the polypeptide of this invention (FCRP). Preferred are monoclonal antibodies.

for the disclosure of this paragraph see [0381.1.1.1] above.

In one embodiment, the present invention relates to a polypeptide as depicted in the respective line in Table II, especially in Table IIA or especially in Table IIB, application no. 6, columns 5 or 8, or fragment or homolog thereof or as coded by the nucleic acid molecule shown in the respective line in Table I, especially Table IA or especially Table IB, application no. 6, columns 5 or 8, preferably the coding region thereof, or fragment or homolog thereof.

In an embodiment, the present invention relates to a polypeptide comprising or consisting of a consensus sequence shown in the respective line in Table IV, application no. 6, column 8 or at least one polypeptide motif shown in the respective line in Table IV, application no. 6, column 8.

In an embodiment thereof, the present invention relates to a polypeptide comprising or consisting of a consensus sequence shown in the respective line in Table IV, application no. 6, column 8 or comprising at least one polypeptide motif shown in the respective line in Table IV, application no. 6, column 8, whereby not more than 20, 15, 10, 9, 8, 7, 6, 5, 4, 3, 2, or 1, or 0 of the amino acids can be replaced by any amino acid.

In another embodiment thereof, the present invention relates to a polypeptide comprising or consisting of a consensus sequence shown in the respective line in Table IV, application no. 6, column 8 or comprising at least one polypeptide motif shown in the respective line in Table IV, application no. 6, column 8, whereby not more than 15%, 10%, 5%, 4%, 3%, 2% or1% or 0% of the amino acids defined as distinct specific amino acids are/is replaced by another amino acid.

In another embodiment thereof, the present invention relates to a polypeptide comprising or consisting of a consensus sequence shown in the respective line in Table IV, application no. 6, column 8 or comprising at least one polypeptide motif shown in the respective line in Table IV, application no. 6, column 8, whereby not more than 20, 15, 10, 9, 8, 7, 6, 5, 4, 3, 2, or 1, or 0 amino acids are inserted into the consensus sequence or polypeptide motif.

In an embodiment of the present invention, the polypeptide of the present invention comprises or consists of a consensus sequence or at least one polypeptide motif shown in the respective line in Table IV, application no. 6, column 8, whereby said polypeptide distinguishes over the sequence depicted in the respective line in Table II, application no. 6, columns 5 or 8 by one or more amino acids. In one embodiment, said polypeptide of the invention distinguishes over the sequence shown in the respective line in Table II, application no. 6, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 6, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in the respective line in Table II, application no. 6, columns 5 and 8.

In an embodiment, the invention relates to a polypeptide conferring an increase in the respective fine chemical isoleucine, leucine, or valine in a non-human organism, especially a microorganism or a plant, or a part thereof, being encoded by the nucleic acid molecule of the invention or used in the process of the invention and having a sequence which distinguishes over the sequence as shown in the respective line in Table II, application no. 6, columns 5 or 8 by one or more amino acids (but not exceeding 5%, preferably 4%, 3%, 2%, 1%, 0.5%, 0.2% or 0.1%). In an embodiment, said polypeptide of the invention does not comprise or consist of the sequence shown in the respective line in Table II, application no. 6, columns 5 or 8. In an embodiment, said polypeptide of the present invention is less than 100%, 99.999%, 99.99%, 99.9% or 99% identical. In one embodiment, said polypeptide which differs at least in one or more amino acids (but not exceeding 5%, preferably 4%, 3%, 2%, 1%, 0.5%, 0.2% or 0.1%) from the polypeptide shown in the respective line in Table II, application no. 6, columns 5 and 8 does not comprise a protein of the sequence shown in the respective line in Table II A and/or II B, application no. 6, columns 5 or 8.

In an embodiment, the present invention relates to a polypeptide having the activity of the protein as shown in the respective line in Table II, application no. 6, column 3, which distinguishes over the sequence depicted in the respective line in Table II, application no. 6, columns 5 or 8 by one or more amino acids, preferably by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 6, columns 5 and 8 by not more than 80%, 70%, 60%, 50%, 40%, 30%, 20%, 10%, 5%, 4%, 3%, 2%, 1% or 0.5%.

In another embodiment the polypeptide of the invention takes the form of a preprotein consisting of a plastidial or mitochondrial transit peptide joint to a polypeptide having the activity of the protein as shown in Table II, column 3, from which the transit peptide is preferably cleaved off upon transport of the preprotein into the organelle for example into the plastid or mitochondrion.

In another embodiment the polypeptide of the invention takes not the form of a preprotein.

[0390.1.1.6] to [0391.1.1.6] for the disclosure of these paragraphs see [0390.1.1.1] to [0391.1.1.1] above.

A polypeptide of the invention can participate in the process of the present invention. The polypeptide or a portion thereof comprises preferably an amino acid sequence, which is sufficiently homologous to an amino acid sequence shown in the respective line in Table II A and/or II B, application no. 6, columns 5 or 8.

Further, the polypeptide can have an amino acid sequence which is encoded by a nucleotide sequence which hybridizes, preferably hybridizes under stringent conditions as described above, to a nucleotide sequence of the nucleic acid molecule of the present invention. Accordingly, the polypeptide has an amino acid sequence which is encoded by a nucleotide sequence that is at least about 35%, 40%, 45%, 50%, 55%, 60%, 65%, 70%, 75%, 80%, 85%, 90%, 91%, 92%, 93%, 94%, 95%, 96%, 97%, 98%, 99%, 99.5% or more homologous to one of the amino acid sequences as shown in the respective line in Table II A and/or II B, application no. 6, columns 5 or 8, or fragments thereof. The preferred polypeptide of the present invention preferably possesses at least one of the activities according to the invention and described herein. A preferred polypeptide of the present invention includes an amino acid sequence encoded by a nucleotide sequence which hybridizes, preferably hybridizes under stringent conditions, to a nucleotide sequence shown in the respective line in Table I A and/or I B, application no. 6, columns 5 or 8, preferably the coding region thereof, or fragments thereto or which is homologous thereto, as defined above.

The polypeptide of the present invention can vary from the sequences shown in the respective line in Table II A and/or II B, application no. 6, columns 5 or 8 in amino acid sequence due to natural variation or mutagenesis, as described in detail herein. Accordingly, the polypeptide comprise an amino acid sequence which is at least about 35%, 40%, 45%, 50%, 55%, 60%, 65%, 70%, 75%, 80%, 85% or 90, 91%, 92%, 93%, 94%, 95%, 96%, 97%, 98%, 99%, 99.5% or more homologous to an entire amino acid sequence shown in the respective line in Table II A and/or II B, application no. 6, columns 5 or 8.

Biologically active portions of a polypeptide of the present invention include peptides comprising amino acid sequences derived from the amino acid sequence of the polypeptide of the present invention or used in the process of the present invention, e.g., the amino acid sequence shown in the respective line in Table II, application no. 6, columns 5 or 8 or the amino acid sequence of a protein homologous thereto, which include fewer amino acids than a full length polypeptide of the present invention or used in the process of the present invention or the full length protein which is homologous to a polypeptide of the present invention or used in the process of the present invention depicted herein, and exhibit at least one activity of a polypeptide of the present invention or used in the process of the present invention.

for the disclosure of this paragraph see [0396.1.1.1] above.

Manipulation of the nucleic acid molecule of the invention may result in the production of a protein having differences from the protein as shown in the respective line in Table II, application no. 6, column 5 or 8. Differences shall mean at least one amino acid different from the sequences as shown in the respective line in Table II, application no. 6, column 5 or 8, preferably at least 2, 3, 4, 5, 6, 7, 8, 9 or 10 amino acids (especially but not exceeding 0.1%, preferably 0.2%, 0.5%, 1%, 2%, 3% 4% or 5%), more preferably at least 15, 20, 25, 30, 35, 40, 45 or 50 amino acids (especially but not exceeding 0.5%, preferably 1%, 2%, 3% 4%, 5%, 10% or 25%) different from the sequences as shown in the respective line in Table II, application no. 6, column 5 or 8. These proteins may be improved in efficiency or activity, or may be more stable and therefore present in greater numbers in the cell as compared to the wild-type cell.

Any mutagenesis strategies for the polypeptide of the present invention or the polypeptide used in the process of the present invention to result in increasing said activity are not meant to be limiting; variations on these strategies will be readily apparent to one skilled in the art. Using such strategies, and incorporating the mechanisms disclosed herein, the nucleic acid molecule and polypeptide of the invention may be utilized to generate algae, ciliates, fungi, other microorganisms like C. glutamicum or plants or parts thereof, expressing proteins as shown in the respective line in Table II, application no. 6, column 5 or 8, or mutated proteins thereof. The nucleic acid molecules and polypeptide molecules of the invention are expressed such that the yield, production, and/or efficiency of production of a desired compound is improved.

[0399.1.1.6] to [0401.1.1.6] for the disclosure of these paragraphs see [0399.1.1.1] to [0401.1.1.1] above.

In one embodiment, a protein (=polypeptide) as shown in the respective line in Table II, application no. 6, column 5 or 8 refers to a polypeptide having an amino acid sequence corresponding to the polypeptide of the invention, especially to a polypeptide as shown in Table II, application no. 6, columns 5 or 8 or homologs thereof, or used in the process of the invention, whereas a “non-inventive protein or polypeptide” or “other polypeptide” refers to a polypeptide having an amino acid sequence corresponding to a protein which is not substantially homologous to a polypeptide of the invention, preferably which is not substantially homologous to a polypeptide or protein as shown in the respective line in Table II, application no. 6, column 5 or 8 or homologs thereof, e.g., a protein which does not confer the activity described herein and which is derived from the same or a different organism.

[0403.1.1.6] to [0409.1.1.6] for the disclosure of these paragraphs see [0403.1.1.1] to [0409.1.1.1] above.

As the nucleic acid molecules of the invention and/or the expression thereof is related to the synthesis of the respective fine chemical isoleucine, leucine, or valine its function as a probe extends to the detection of microorganisms, plant tissues, plants, plant variets, plant ecotypes or plant genera with varying, advantageously increased, capability or potential for synthesis of the respective fine chemical isoleucine, leucine, or valine. Therefore in one embodiment the present invention relates to a method for analyzing the capability or potential of a plant tissue, a plant, a plant variety or ecotype to produce the respective fine chemical isoleucine, leucine, or valine by using the respective antibody of the invention as a probe to detect the amount of the polypeptide encoded by said nucleic acid molecule of the invention in a non-human organism in comparison to another organism.

Therefore in one embodiment the invention relates to a method to probe a non-human organism for its capability for the production of the respective fine chemical comprising the steps of
a) analyzing the sequence and/or the expression of the respective nucleic acid molecule of the invention in least two non-human organisms which differ in the capability to produce the respective fine chemical;
b) associate the different sequence and/or expression level of the nucleic acid molecules of the invention with their different capability to produce the respective fine chemical;
c) analyze the sequence and/or the expression of the nucleic acid molecule of the invention in at least a third non-human organism;
d) predict the capability of the at least third organism to produce the respective fine chemical based by comparing the sequence and/or expression of the nucleic acid of the invention to the sequences and/or expression levels of the nucleic acid molecule of the invention of the at least two organisms analyzed in step a.

[0411.1.1.6] to [0430.1.1.6] for the disclosure of these paragraphs see [0411.1.1.1] to [0430.1.1.1] above.

Accordingly the present invention relates to any cell transgenic for any nucleic acid characterized as part of the invention, e.g. conferring the increase of the respective fine chemical isoleucine, leucine, or valine in a cell or a non-human organism or a part thereof, e.g. the nucleic acid molecule of the invention, the nucleic acid construct of the invention, the vector of the invention, the expression cassette according to the invention, or a nucleic acid molecule encoding the polypeptide of the invention, e.g. encoding a polypeptide having an activity as the protein as shown in the respective line in Table II, application no. 6, column 3. Due to the above-mentioned activity the respective fine chemical isoleucine, leucine, or valine content in a cell or a non-human organism is increased. For example, due to modulation or manipulation, the cellular activity is increased, in a preferred embodiment in organelles such as plastids or mitochondria, e.g. due to an increased expression or specific activity or specific targeting of the subject matters of the invention in a cell or a non-human organism or a part thereof especially in organelles such as plastids or mitochondria, or in another embodiment in the cytosol. Transgenic for a polypeptide having a protein or a protein activity means herein that due to modulation or manipulation of the genome, the activity of protein as shown in the respective line in Table II, application no. 6, column 3 or a protein as shown in the respective line in Table II, application no. 6, column 3-like activity is increased in the cell or non-human organism or part thereof, especially in organelles such as plastids or mitochondria, or especially in the cytosol. Examples are described above in context with the process of the invention.

for the disclosure of this paragraph see [0432.1.1.1] above.

A naturally occurring expression cassette—for example the naturally occurring combination of the promoter of the gene encoding a protein as shown in the respective line in Table II, application no. 6, column 5 or 8, preferably the coding region thereof, or fragments or homologs thereof with the corresponding encoding gene—becomes a transgenic expression cassette when it is modified by non-natural, synthetic “artificial” methods such as, for example, mutagenesis. Such methods have been described (U.S. Pat. No. 5,565,350; WO 00/15815; also see above).

[0434.1.1.6] to [0435.1.1.6] for the disclosure of these paragraphs see [0434.1.1.1] to [0435.1.1.1] above.

Transgenic plants comprising the respective fine chemical(s) synthesized in the process according to the invention can be marketed directly without isolation of the fine chemical synthesized. In the process according to the invention, plants are understood as meaning all plant parts, plant organs such as leaf, stalk, root, tubers or seeds or propagation material or harvested material or the intact plant. In this context, the seed encompasses all parts of the seed such as the seed coats, epidermal cells, seed cells, endosperm or embryonic tissue. The fine chemical produced in the process according to the invention may, however, also be isolated from the plant. In case of isoleucine, leucine, or valine this can be in free form or bound form. Fine chemical(s) produced by this process can be harvested by harvesting the non-human organisms either from the culture in which they grow or from the field. For example, this can be done via squeezing, grinding and/or extraction, salt precipitation and/or ionexchange chromatography of the plant parts, preferably the plant seeds, plant fruits, plant tubers and the like.

[0437.1.1.6] to [0440.1.1.6] for the disclosure of these paragraphs see [0437.1.1.1] to [0440.1.1.1] above.

[0442.1.1.6] to [0454.1.1.6] for the disclosure of these paragraphs see [0442.1.1.1] to [0454.1.1.1] above.

In one embodiment, the present invention relates to a method for the identification of a gene product conferring the production of or an increase in the fine chemical production in a non-human cell, comprising the following steps:
(a) contacting, e.g. hybridizing, a, some or all nucleic acid molecules of a sample, e.g. cells, tissues, plants or microorganisms or a nucleic acid library, which can contain a candidate gene encoding a gene product conferring the production of or an increase in the fine chemical after expression, with the nucleic acid molecule of the present invention as shown in the respective line in column 5 or 8 of Table I A or B of application no. 6, preferably the coding region thereof, or a fragment or homolog thereof;
(b) identifying the nucleic acid molecules, which hybridize under relaxed stringent conditions with the nucleic acid molecule of the present invention in particular to the nucleic acid molecule sequence shown in the respective line in Table I, application no. 6, columns 5 or 8, preferably the coding region thereof, of a fragment or a homolog thereof, preferably in Table I B, application no. 6, columns 5 or 8, preferably the coding region thereof, of a fragment or a homolog thereof, and, optionally, isolating the full length cDNA clone or complete genomic clone;
(c) introducing the candidate nucleic acid molecules or a fragment thereof in host cells, preferably in a plant cell or a microorganism, appropriate for producing the fine chemical;
(d) expressing the identified nucleic acid molecules in the host cells for which the production of or the increased production of the fine chemical is desired;
(e) assaying the fine chemical level in the non-human host cells; and
(f) identifying the nucleic acid molecule and/or its gene product which expression confers the production of or an increase in the fine chemical level in the non-human host cell after expression compared to the wild type.

for the disclosure of this paragraph see [0456.1.1.1] above.

In another embodiment, the present invention relates to a method for the identification of a gene product conferring the production of or an increase in the fine chemical production in a cell, comprising the following steps:
(a) identifying a nucleic acid molecule of a non-human organism, which is at least 20%, 25%, 30%, 35%, 40%, 50%, 60%, 70%, 80%, 90%, 95%, 96%, 97%, 98%, 99%, 99.5% or more homolog to the nucleic acid molecule encoding a polypeptide comprising the polypeptide molecule as shown in the respective line in column 5 or 8 of Table II, application no. 6, or comprising a consensus sequence or a polypeptide motif as shown in the respective line in column 8 of Table IV, application no. 6, or being encoded by a nucleic acid molecule comprising a polynucleotide as shown in the respective line in column 5 or 8 of Table I, application no. 6, preferably the coding region thereof, or a homologue thereof as described herein, for example via homology search in a data bank;
(b) introducing the candidate nucleic acid molecule in non-human host cells, preferably in a plant cell or a microorganism, appropriate for producing the fine chemical;
(c) expressing the identified nucleic acid molecule in the non-human host cells;
(d) assaying the fine chemical level in the non-human host cells; and
(e) identifying the nucleic acid molecule and its gene product which expression confers the production of or an increase in the fine chemical level in the non-human host cell after expression compared to the wild type.

for the disclosure of this paragraph see [0458.1.1.1] above.

Further, the nucleic acid molecules disclosed herein, in particular the nucleic acid molecules shown in the respective line in column 5 or 8 of Table I A or B of application no. 6, may be sufficiently homologous to the sequences of related species such that these nucleic acid molecules may serve as markers for the construction of a genomic map in related organism or for association mapping. Furthermore natural variation in the genomic regions corresponding to nucleic acids disclosed herein, in particular the nucleic acid molecule shown in the respective line in column 5 or 8 of Table I A or B, of application no. 6 or homologous thereof, may lead to variation in the activity of the proteins disclosed herein, in particular the proteins comprising polypeptides as shown in the respective lines in column 5 or 8 of Table II A or B, of application no. 6 or their homologous, or comprising the consensus sequence or the polypeptide motif as shown in the respective line in column 8 of Table IV, and in consequence in a natural variation of an increased fine chemical production.

In consequence natural variation eventually also exists in form of more active allelic variants leading already to a relative increase in the fine chemical. Different variants of the nucleic acid molecules disclosed herein, in particular the nucleic acid molecules comprising the nucleic acid molecules as shown in the respective line in column 5 or 8 of Table I A or B of application no. 6, preferably the coding region thereof, which corresponds to different levels of increase in fine chemical, e.g. different levels of increase in fine chemical, can be indentified and used for marker assisted breeding for an enhanced production of the fine chemical.

Natural variation may also exist in the regulatory regions, e.g. the promoters of the natural genes comprising the nucleic acid molecules as shown in the respective line column 5 or 8 of Table I A or B of application no. 6, preferably the coding region thereof, which can similarly correspond to different levels of increase in the fine chemical, e.g. different levels of increase in the fine chemical can be identified and used for marker assisted breeding for an enhanced production of the fine chemical.

Accordingly, the present invention relates to a method for breeding plants for the production of the fine chemical, comprising
(a) selecting a first plant variety with an increased production of the fine chemical, e.g. based on an increased expression of a nucleic acid molecule of the invention as disclosed herein, in particular of a nucleic acid molecule comprising a nucleic acid molecule as shown in the respective line in column 5 or 8 of Table I A or B of application no. 6, preferably the coding region thereof, or fragments or homologs thereof, or a polypeptide comprising a polypeptide as shown in the respective line in column 5 or 8 of Table II A or B of application no. 6, or fragments or homologs thereof, or comprising a consensus sequence or a polypeptide motif as shown in the respective line in column 8 of Table IV, or a homolog thereof, as described herein;
(b) associating the level of increased production of the fine chemical, with the expression level or the genomic structure of a gene encoding said polypeptide or said nucleic acid molecule;
(c) crossing the first plant variety with a second plant variety, which significantly differs in its level of fine chemical; and
(d) identifying, which of the offspring varieties has got increased production of the fine chemical by the expression level of said polypeptide or nucleic acid molecule or the genomic structure of the gene(s) encoding said polypeptide or nucleic acid molecule of the invention.

[0462.1.1.6] to [0482.1.1.6] for the disclosure of these paragraphs see [0462.1.1.1] to [0482.1.1.1] above.

[0482.2.6.6] [0482.3.6.6] In a further embodiment the present invention is characterized by the features as disclosed in the following items:

Item 1. A process for the production of the fine chemical isoleucine, leucine or valine, respectively
(a) increasing or generating one or more activities selected from the group consisting of 2,3-dihydroxyphenylpropionate 1,2-dioxygenase, 2-isopropylmalate synthase, 3-isopropylmalate dehydrogenase, 47266012-protein, 50S ribosomal protein L14, ABC transporter ATP binding component, ABC transporter ATP-binding protein, ABC transporter component, ABC transporter permease protein, acetolactate synthase, acetolactate synthase small subunit, acetyl CoA carboxylase, acetyl-CoA acetyltransferase, acetyltransferase, acid shock protein, acyl transferase, acyltransferase, adenylate kinase, adenylosuccinate lyase, agmatinase, aldehyde dehydrogenase, aminotransferase, anthranilate synthase component II, arginine exporter protein, At1g19800-protein, At1g23110-protein, At1g47380-protein, At4g32480-protein, At5g16650-protein, ATP-binding component of a transport system, auxin response factor, b0050-protein, b1003-protein, b1163-protein, b1445-protein, b1522-protein, b1670-protein, b1672-protein, b2513-protein, b2613-protein, b2673-protein, b2739-protein, b2812-protein, b3083-protein, b3490-protein, b3989-protein, b4029-protein, b4050-protein, b4056-protein, b4263-protein, beta-galactosidase, beta-hydroxylase, bifunctional protein (phosphoribosyltransferase and regulatory protein), binding-protein-dependent transport systems inner membrane component, branched chain aminotransferase, branched-chain amino acid permease, branched-chain aminoacid aminotransferase, calcium-dependent protein kinase, carbamoyl-phosphate synthase subunit, CBL-interacting protein kinase, cell division control protein, chaperone protein CIpB, cullin, cyclin, cyclin D, cytochrome c-type protein, dihydroxyacid dehydratase, disease resistance protein, DNA helicase II, DNA helicase IV, DNA mismatch repair protein, DNA polymerase, DNA-binding protein, elongation factor Tu, enoyl-CoA hydratase, eukaryotic translation initiation factor, eukaryotic translation initiation factor 5, ferredoxin, flavodoxin, fumarylacetoacetate hydrolase, GDP-mannose 4,6-dehydratase, geranylgeranyl pyrophosphate synthase, gluconate transport system permease 3, glucose-1-phosphate cytidylyltransferase, glucose-6-phosphate 1-dehydrogenase, glutamate-ammonia-ligase, glutamine synthetase, glutaredoxin, glycerol-3-phosphate dehydrogenase, glycogen (starch) synthase, glycogen synthase, glycoprotease, glycosyl transferase, GM02LC11114-protein, GM02LC5744-protein, H/ACA ribonucleoprotein complex subunit 3, harpin-induced family protein, heat shock transcription factor, hexose transporter, histone H2A, homocitrate synthase, homoserine dehydrogenase, hydrolase, iron(III) dicitrate-binding protein, isochorismate synthase, isopropylmalate isomerase large subunit, ketol-acid reductoisomerase, L-serine dehydratase, MADS-box transcription factor, major facilitator superfamily transporter protein, malate dehydrogenase, malic enzyme, membrane protein, membrane transport protein, methylglyoxal synthase, mitochondrial dicarboxylate transporter, monothiol glutaredoxin, murein transglycosylase, Na+/K+ transporter, NAD(P)H-quinone oxidoreductase subunit, NADH-quinone oxidoreductase subunit, NADP-dependent malic enzyme, Nucleoside monophosphate phosphohydrolase, O-antigen chain length determinant, oxidoreductase, oxireductase, peptidase, phosphate transporter, phosphoadenosine phosphosulfate reductase, phosphoanhydride phosphorylase, phosphoglycerate kinase, phosphopantetheine adenylyltransferase, photosystem II protein, polygalacturonase, polyphosphate kinase, precorrin methylase, protein kinase, protein phosphatase, protoporphyrin oxidase, purine nucleoside phosphorylase, purine-nucleoside phosphorylase, pyruvate kinase, riboflavin biosynthesis protein, ribosephosphate isomerase, constitutive, RNA binding protein, RNA-binding protein, Sec-independent protein translocase subunit, serine hydroxymethyltransferase, short-chain alcohol dehydrogenase family, sll1032-protein, sll1455-protein, sll1761-protein, superoxide dismutase, thioredoxin, thioredoxin family protein, threonine dehydratase, threonine synthase, transcription factor, transcriptional regulator, transport protein, trehalose-phosphatase, TTC1386-protein, urease subunit, vacuolar ATPase subunit, valine-pyruvate transaminase, YAL013W-protein, ydr183w-protein, ygl237c-protein, YGR193C-protein, yhr006w-protein, YKL038W-protein, YLR224W-protein, zinc finger protein, zinc transporter, and Zm_{—}4842_BE510522-protein, in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the respective production of isoleucine, leucine or valine, respectively, or a composition comprising isoleucine, leucine or valine, respectively, in said non-human organism or in the culture medium surrounding said non-human organism.

Item 2. A process for the production of a respective fine chemical isoleucine, leucine or valine, which comprises
(A)
(i) increasing or generating of the expression of; and/or
(ii) increasing or generating the expression of an expression product of; and/or
(iii) increasing or generating one or more activities of an expression product encoded by;
at least one respective nucleic acid molecule comprising a nucleic acid molecule selected from the group consisting of:
(a) a nucleic acid molecule encoding the polypeptide shown in column 5 or 8 of Table II, preferably Table II B, application no.6, or a homolog or a fragment thereof, whereby the respective line discloses in column 7 isoleucine, leucine or valine, respectively;
(b) a nucleic acid molecule shown in column 5 or 8 of Table I, preferably Table I B, application no.6, or a homolog or a fragment thereof (preferably the coding region thereof) , whereby the respective line discloses in column 7 isoleucine, leucine or valine, respectively;
(c) a nucleic acid molecule, which, as a result of the degeneracy of the genetic code, can be derived from a polypeptide sequence depicted in column 5 or 8 of Table II, preferably Table II B, application no.6, whereby the respective line discloses in column 7 isoleucine, leucine or valine, respectively;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 6, or the coding region thereof , whereby the respective line discloses in column 7 isoleucine, leucine or valine, respectively;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no.6, whereby the respective line discloses in column 7 isoleucine, leucine or valine, respectively;
(i) a nucleic acid molecule encoding a polypeptide having the activity represented by a respective protein as depicted in column 5 of Table II, application no. 6, whereby the respective line discloses in column 7 isoleucine, leucine or valine, respectively;
(j) a nucleic acid molecule which comprises a polynucleotide, which is obtained by amplifying a cDNA library or a genomic library using the primers as depicted in column 8 of Table III, application no. 6, whereby the respective line discloses in column 7 isoleucine, leucine or valine, respectively; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto; in a non-human organism or a part thereof, preferably a microorganism, a plant cell, a plant or a part thereof, as compared to a corresponding non-transformed wild type non-human organism or a part thereof; and
(B) growing the non-human organism or a part thereof under conditions which permit the respective production of isoleucine, leucine or valine or a composition comprising isoleucine, leucine or valine, respectively, in said non-human organism or in the culture medium surrounding said non-human organism.

Item 3. A process of items 1 or 2, comprising of recovering isoleucine, leucine or valine in its free or bound form.

Item 4. The process of any one of items 1 to 3, comprising the following steps:
(a) selecting a non-human organism or a part thereof expressing a polypeptide encoded by the nucleic acid molecule characterized in item 2;
(b) mutagenizing the selected non-human organism or the part thereof;
(c) comparing the activity or the expression level of said polypeptide in the mutagenized non-human organism or the part thereof with the activity or the expression of said polypeptide of the selected non-human organisms or the part thereof;
(d) selecting the mutated non-human organisms or parts thereof, which comprise an increased activity or expression level of said polypeptide compared to the selected non-human organism or the part thereof;
(e) optionally, growing and cultivating the non-human organisms or the parts thereof; and
(f) recovering, and optionally isolating, the free or bound isoleucine, leucine or valine, respectively, produced by the selected mutated non-human organisms or parts thereof.

Item 5. The process of any one of items 1 to 4 wherein the activity of said protein or the expression of said nucleic acid molecule is increased or generated transiently or stably.

Item 6. An isolated nucleic acid molecule comprising a nucleic acid molecule selected from the group consisting of:
(a) a nucleic acid molecule encoding a polypeptide shown in Table II, application no. 6, column 5 or 8, preferably shown in Table II B, application no. 6, column 8, or a homolog or a fragment thereof;
(b) a nucleic acid molecule shown in Table I, application no. 6, column 5 or 8, preferably shown in Table I B, application no. 6, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 6, preferably in column 8 of Table II B, application no. application no.;
(d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 3, preferably shown in column 8 of Table I B, application no. 6, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 6;
(i) a nucleic acid molecule encoding a polypeptide having the activity represented by a respective protein as depicted in column 5 of Table II, application no. 6;
(j) a nucleic acid molecule comprising a polynucleotide which is obtained by amplifying a cDNA library or a genomic library using the primers in Table III, application no. 6, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j) ;
or a nucleic acid molecule comprising a sequence which is complementary thereto.

Item 7. A nucleic acid construct which confers the expression of the nucleic acid molecule as defined in item 6, comprising one or more regulatory elements.

Item 8. An expression cassette comprising
(a) a promoter, preferably selected from the group consisting of Big35S, PCUbi, Super and USP;
(b) a nucleic acid molecule as defined in item 6.

Item 9. A vector comprising the nucleic acid molecule as defined in item 6 or the nucleic acid construct as claimed in item 7 or the expression cassette as claimed in item 8.

Item 10. A host cell or a plant organelle, which has been transformed with the vector as claimed in item 9 or the nucleic acid molecule as defined in item 6 or the nucleic acid construct as claimed in item 7 or the expression cassette as claimed in item 8.

Item 11. The host cell of item 10, which is a plant cell or a microorganism.

Item 12. A process for producing a polypeptide, wherein the polypeptide is expressed in a host cell as claimed in item 10 or 11.

Item 13. A polypeptide produced by the process as claimed in item 12 or encoded by the nucleic acid molecule as defined in item 6.

Item 14. An antibody, which binds specifically to the polypeptide as claimed in item 13.

Item 15. A plant tissue, propagation material, harvested material or a plant comprising the host cell as claimed in item 10 or 11.

Item 16. A process for the identification of a compound conferring an increase in isoleucine, leucine or valine production, respectively, in a non-human organism, comprising the steps:
(a) culturing a plant cell or tissue or microorganism or maintaining a plant expressing the polypeptide encoded by the nucleic acid molecule as defined in item 6 conferring an increase in the amount of the respective isoleucine, leucine or valine in a non-human organism or a part thereof and a readout system capable of interacting with the polypeptide under suitable conditions which permit the interaction of the polypeptide with said readout system in the presence of a compound or a sample comprising a plurality of compounds and capable of providing a detectable signal in response to the binding of a compound to said polypeptide under conditions which permit the expression of said readout system and of the polypeptide encoded by the nucleic acid molecule of item 6 conferring an increase in the amount of the respective isoleucine, leucine or valinein a non-human organism or a part thereof;
(b) identifying if the compound is an effective agonist by detecting the presence or absence or increase of a signal produced by said readout system.

Item 17. A method for the production of an agricultural composition comprising the steps of the method of item 16 and formulating the compound identified in item 16 in a form acceptable for an application in agriculture.

Item 18. A composition comprising the nucleic acid molecule as defined in item 6, the polypeptide as claimed in item 13, the nucleic acid construct as claimed in item 7, the expression cassette as claimed in item 8, the vector as claimed in item 9, the compound as claimed in item 16, the antibody as claimed in item 14, and optionally an agricultural acceptable carrier.

Item 19. Use of the nucleic acid molecule as defined in item 6 for the identification of a nucleic acid molecule conferring an increase in isoleucine, leucine or valine, respectively, after expression.

Item 20. Cosmetic, pharmaceutical, food or feed composition comprising the nucleic acid moleculeas defined in item 6, the polypeptide as claimed in item 13, the nucleic acid construct as claimed in item 7, the expression cassette as claimed in item 8, the vector as claimed in item 9, the antibody as claimed in item 14 , the plant or a part thereof, or plant tissue as claimed in item 15, the harvested material or propagation material as claimed in item 15 or the host cell of item as claimed in items 10 or 11.

Item 21. Use of the nuclei acid molecule as defined in item 6, the polypeptide as claimed in item 13, the nucleic acid construct as claimed in item 7, the expression cassette as claimed in item 8, the vector as claimed in claim9, the plant or plant tissue as claimed in item 15, or the host cell of item 10 to 11 for the production of plant resistant to a herbicide inhibiting the production of isoleucine, leucine or valine, respectively.

[0483.1.1.6] to [0494.1.1.6] for the disclosure of these paragraphs see [0483.1.1.1] to [0494.1.1.1] above.

### Example 11a

### PCR Amplification of the Sequences

Unless otherwise specified, standard methods as described in Sambrook et al., Molecular Cloning: A laboratory manual, Cold Spring Harbor 1989, Cold Spring Harbor Laboratory Press are used.

The inventive sequences as shown in the respective line in Table I, column 5, preferably the coding region thereof, were amplified by PCR as described in the protocol of the Pfu Ultra, Pfu Turbo or Herculase DNA polymerase (Stratagene). The composition for the protocol of the Pfu Ultra, Pfu Turbo or Herculase DNA polymerase was as follows: 1×PCR buffer (Stratagene), 0.2 mM of each dNTP, 100 ng genomic DNA of Saccharomyces cerevisiae (strain S288C; Research Genetics, Inc., now Invitrogen), Escherichia coli (strain MG1655; E. coli Genetic Stock

Center), Synechocystis sp. (strain PCC6803), Azotobacter vinelandii (strain N.R. Smith,16), Thermus thermophilus (HB8) or 50 ng cDNA from various tissues and development stages of Arabidopsis thaliana (ecotype Columbia), Physcomitrella patens, Glycine max (variety Resnick), Brassica napus, Oryza sativa or Zea mays (variety B73, Mo17, A188), 50 pmol forward primer, 50 pmol reverse primer, with or without 1 M Betaine, 2.5 u Pfu Ultra, Pfu Turbo or Herculase DNA polymerase.

The amplification cycles were as follows:

1 cycle of 2-3 minutes at 94-95° C., then 25-36 cycles with 30-60 seconds at 94-95° C., 30-45 seconds at 50-60° C. and 210-480 seconds at 72° C., followed by 1 cycle of 5-10 minutes at 72° C., then 4-16° C.—preferably for Saccharomyces cerevisiae, Escherichia coli, Synechocystis sp., Azotobacter vinelandii, Thermus thermophilus.

In case of Arabidopsis thaliana, Brassica napus, Glycine max, Oryza sativa, Physcomitrella patens, Zea mays the amplification cycles were as follows: 1 cycle with 30 seconds at 94° C., 30 seconds at 61° C., 15 minutes at 72° C., then 2 cycles with 30 seconds at 94° C., 30 seconds at 60° C., 15 minutes at 72° C., then 3 cycles with 30 seconds at 94° C., 30 seconds at 59° C., 15 minutes at 72° C., then 4 cycles with 30 seconds at 94° C., 30 seconds at 58° C., 15 minutes at 72° C., then 25 cycles with 30 seconds at 94° C., 30 seconds at 57° C., 15 minutes at 72° C., then 1 cycle with 10 minutes at 72° C., then finally 4-16° C.

RNAs were generated with the RNeasy Plant Kit according to the standard protocol (Qiagen) and Superscript II Reverse Transkriptase was used to produce double stranded cDNA according to the standard protocol (Invitrogen).

ORF specific primer pairs for the genes to be expressed are shown in the respective line in Table III, column 8. The following adapter sequences were added to Saccharomyces cerevisiae ORF specific primers (see Table III) for cloning purposes:

SEQ ID NO: 20
i)foward primer: 5′-GGAATTCCAGCTGACCACC-3′
SEQ ID NO: 21
ii) reverse primer: 5′-GATCCCCGGGAATTGCCATG-3′

These adaptor sequences allow cloning of the ORF into the various vectors containing the Resgen adaptors, see table column E of Table c.

The following adapter sequences were added to Saccharomyces cerevisiae, Escherichia coli, Synechocystis sp., Azotobacter vinelandii, Thermus thermophilus, Arabidopsis thaliana, Brassica napus, Glycine max, Oryza sativa , Physcomitrella patens, or Zea mays ORF specific primers for cloning purposes:

SEQ ID NO: 22
iii) forward primer: 5′-TTGCTCTTCC- 3′
SEQ ID NO: 23
iiii) reverse primer: 5′-TTGCTCTTCG-3′

The adaptor sequences allow cloning of the ORF into the various vectors containing the Colic adaptors, see table column E of Table c.

Therefore for amplification and cloning of Saccharomyces cerevisiae SEQ ID NO: 62717, a primer consisting of the adaptor sequence i) and the ORF specific sequence SEQ ID NO: 63159 and a second primer consisting of the adaptor sequence ii) and the ORF specific sequence SEQ ID NO: 63160 were used.

For amplification and cloning of Escherichia coli SEQ ID NO: 6818, a primer consisting of the adaptor sequence iii) and the ORF specific sequence SEQ ID NO: 7076 and a second primer consisting of the adaptor sequence iiii) and the ORF specific sequence SEQ ID NO: 7077 were used.

For amplification and cloning of Synechocystis sp. SEQ ID NO: 12140, a primer consisting of the adaptor sequence iii) and the ORF specific sequence SEQ ID NO: 12336 and a second primer consisting of the adaptor sequence iiii) and the ORF specific sequence SEQ ID NO: 12337 were used.

For amplification and cloning of Arabidopsis thaliana SEQ ID NO: 385, a primer consisting of the adaptor sequence iii) and the ORF specific sequence SEQ ID NO: 623 and a second primer consisting of the adaptor sequence iiii) and the ORF specific sequence SEQ ID NO: 624 were used.

For amplification and cloning of Azotobacter vinelandii SEQ ID NO: 5557, a primer consisting of the adaptor sequence iii) and the ORF specific sequence SEQ ID NO: 6033 and a second primer consisting of the adaptor sequence iiii) and the ORF specific sequence SEQ ID NO: 6034 were used.

For amplification and cloning of Brassica napus SEQ ID NO: 15532, a primer consisting of the adaptor sequence iii) and the ORF specific sequence SEQ ID NO: 16150 and a second primer consisting of the adaptor sequence iiii) and the ORF specific sequence SEQ ID NO: 16151 were used.

For amplification and cloning of Glycine max SEQ ID NO: 10811, a primer consisting of the adaptor sequence iii) and the ORF specific sequence SEQ ID NO: 11207 and a second primer consisting of the adaptor sequence iiii) and the ORF specific sequence SEQ ID NO: 11208 were used.

For amplification and cloning of Physcomytrella patens SEQ ID NO: 45757, a primer consisting of the adaptor sequence iii) and the ORF specific sequence SEQ ID NO: 45791 and a second primer consisting of the adaptor sequence iiii) and the ORF specific sequence SEQ ID NO: 45792 were used.

For amplification and cloning of Thermus thermophilus SEQ ID NO: 12698, a primer consisting of the adaptor sequence iii) and the ORF specific sequence SEQ ID NO: 12970 and a second primer consisting of the adaptor sequence iiii) and the ORF specific sequence SEQ ID NO: 12971 were used.

For amplification and cloning of Zea mays SEQ ID NO: 68413, a primer consisting of the adaptor sequence iii) and the ORF specific sequence SEQ ID NO: 68531 and a second primer consisting of the adaptor sequence iiii) and the ORF specific sequence SEQ ID NO: 68532 were used.

Following these examples every sequence disclosed in Table I, preferably column 5, especially the coding region thereof can be cloned by fusing the adaptor sequences to the respective specific primers sequences as disclosed in Table III, column 8 using the respective vectors shown in Table c.

[0496.1.1.6] to [0499.1.1.6] for the disclosure of these paragraphs see [0496.1.1.1] to [0499.1.1.1] above.

### Example 11e

### Cloning of Inventive Sequences as Shown in Table I, Column 5 in the Different Expression Vectors

For cloning of for example SEQ ID NO: 6818 from Escherichia coli or any other ORF from Escherichia coli, Synechocystis sp., Azotobacter vinelandii, Thermus thermophilus, Arabidopsis thaliana, Brassica napus, Glycine max, Physcomitrella patens, or Zea mays the vector DNA was treated with the restriction enzymes Pacl and Ncol following the standard protocol (MBI Fermentas).

For cloning for example the ORFs of SEQ ID NO: 62717 from Saccharomyces cerevisiae or any other ORF from Saccharomyces cerevisiae into vectors containing the Resgen adaptor sequence the respective vector DNA was treated with the restriction enzyme Ncol.

For cloning of for example ORFs SEQ ID NO: 14885 from Saccharomyces cerevisiae or any other ORF from Saccharomyces cerevisiae into vectors containing the Colic adaptor sequence, the respective vector DNA was treated with the restriction enzymes Pacl and Ncol following the standard protocol (MBI Fermentas).

In all cases the reaction was stopped by inactivation at 70° C. for 20 minutes and purified over QIAquick or NucleoSpin Extract II columns following the standard protocol (Qiagen or Macherey-Nagel).

Then the PCR-product representing the amplified ORF with the respective adapter sequences and the vector DNA were treated with T4 DNA polymerase according to the standard protocol (MBI Fermentas) to produce single stranded overhangs with the parameters 1 unit T4 DNA polymerase at 37° C. for 2-10 minutes for the vector and 1-2 u T4 DNA polymerase at 15-17° C. for 10-60 minutes for the PCR product representing SEQ ID NO: 6818.

The reaction was stopped by addition of high-salt buffer and purified over QIAquick or NucleoSpin Extract II columns following the standard protocol (Qiagen or Macherey-Nagel).

According to this example the skilled person is able to clone all sequences disclosed in Table I, preferably column 5 or column 8, especially the coding region thereof.

Approximately 30-60 ng of prepared vector and a defined amount of prepared amplificate were mixed and hybridized at 65° C. for 15 minutes followed by 37° C. 0,1 ° C./1 seconds, followed by 37° C. 10 minutes, followed by 0,1 ° C./1 seconds, then 4-10 ° C.

The ligated constructs were transformed in the same reaction vessel by addition of competent E. coli cells (strain DHSalpha) and incubation for 20 minutes at 1° C. followed by a heat shock for 90 seconds at 42° C. and cooling to 1-4° C. Then, complete medium (SOC) was added and the mixture was incubated for 45 minutes at 37° C. The entire mixture was subsequently plated onto an agar plate with 0.05 mg/ml kanamycin and incubated overnight at 37° C.

The outcome of the cloning step was verified by amplification with the aid of primers which bind upstream and downstream of the integration site, thus allowing the amplification of the insertion. The amplifications were carried out as described in the protocol of Taq DNA polymerase (Gibco-BRL).

The amplification cycles were as follows:

1 cycle of 1-5 minutes at 94° C., followed by 35 cycles of in each case 15-60 seconds at 94° C., 15-60 seconds at 50-66° C. and 5-15 minutes at 72° C., followed by 1 cycle of 10 minutes at 72° C., then 4-16° C.

Several colonies were checked, but only one colony for which a PCR product of the expected size was detected was used in the following steps.

A portion of this positive colony was transferred into a reaction vessel filled with complete medium (LB) supplemented with kanamycin and incubated overnight at 37° C.

The plasmid preparation was carried out as specified in the Qiaprep or NucleoSpin Multi-96 Plus standard protocol (Qiagen or Macherey-Nagel).

[0501.1.1.6] to [0503.1.1.6] for the disclosure of these paragraphs see [0501.1.1.1] to [0503.1.1.1] above. Table d showing results of plant analyses

TABLE d

| SeqID | Target | Locus | Metabolite | Source | Promotor | Method | Min | Max |
|---|---|---|---|---|---|---|---|---|
| 15187 | non- | 47266012_SOYBEAN | isoleucine | ARA_LEAF | p-PcUBI | GC | 36 | 43 |
| | targeted | | | | | | | |
| 15532 | plastidic | 51340801_CANOLA | leucine | ARA_LEAF | p-PcUBI | GC | 80 | 500 |
| 15532 | plastidic | 51340801_CANOLA | valine | ARA_LEAF | p-PcUBI | GC | 34 | 207 |
| 16263 | plastidic | 59554615_SOYBEAN | valine | ARA_LEAF | p-PcUBI | GC | 22 | 74 |
| 16883 | plastidic | 59582753_SOYBEAN | isoleucine | ARA_LEAF | p-PcUBI | GC | 33 | 45 |
| 16883 | plastidic | 59582753_SOYBEAN | leucine | ARA_LEAF | p-PcUBI | GC | 47 | 54 |
| 16883 | plastidic | 59582753_SOYBEAN | valine | ARA_LEAF | p-PcUBI | GC | 45 | 97 |
| 17451 | non- | At1g07430 | valine | ARA_LEAF | p-PcUBI | GC | 24 | 39 |
| | targeted | | | | | | | |
| 385 | non- | At1g14490 | valine | ARA_LEAF | p-PcUBI | GC | 21 | 54 |
| | targeted | | | | | | | |
| 17637 | non- | At1g19800 | isoleucine | ARA_LEAF | p-PcUBI | GC | 45 | 805 |
| | targeted | | | | | | | |
| 17637 | non- | At1g19800 | leucine | ARA_LEAF | p-PcUBI | GC | 115 | 1238 |
| | targeted | | | | | | | |
| 102086 | non- | At1g23110 | valine | ARA_LEAF | p-PcUBI | GC | 28 | 40 |
| | targeted | | | | | | | |
| 17701 | non- | At1g26830 | isoleucine | ARA_LEAF | p-PcUBI | GC | 45 | 143 |
| | targeted | | | | | | | |
| 17701 | non- | At1g26830 | leucine | ARA_LEAF | p-PcUBI | GC | 59 | 187 |
| | targeted | | | | | | | |
| 17968 | non- | At1g36730 | isoleucine | ARA_LEAF | p-PcUBI | GC | 49 | 162 |
| | targeted | | | | | | | |
| 17968 | non- | At1g36730 | leucine | ARA_LEAF | p-PcUBI | GC | 52 | 149 |
| | targeted | | | | | | | |
| 18070 | non- | At1g43850 | isoleucine | ARA_LEAF | p-PcUBI | GC | 69 | 406 |
| | targeted | | | | | | | |
| 18070 | non- | At1g43850 | leucine | ARA_LEAF | p-PcUBI | GC | 58 | 377 |
| | targeted | | | | | | | |
| 18070 | non- | At1g43850 | valine | ARA_LEAF | p-PcUBI | GC | 58 | 173 |
| | targeted | | | | | | | |
| 18122 | non- | At1g47380 | leucine | ARA_LEAF | p-PcUBI | GC | 51 | 134 |
| | targeted | | | | | | | |
| 18122 | non- | At1g47380 | valine | ARA_LEAF | p-PcUBI | GC | 20 | 105 |
| | targeted | | | | | | | |
| 18235 | non- | At1g48260 | isoleucine | ARA_LEAF | p-PcUBI | GC | 78 | 747 |
| | targeted | | | | | | | |
| 18235 | non- | At1g48260 | leucine | ARA_LEAF | p-PcUBI | GC | 86 | 639 |
| | targeted | | | | | | | |
| 18235 | non- | At1g48260 | valine | ARA_LEAF | p-PcUBI | GC | 51 | 277 |
| | targeted | | | | | | | |
| 18869 | non- | At1g61950 | isoleucine | ARA_LEAF | p-PcUBI | GC | 96 | 174 |
| | targeted | | | | | | | |
| 18869 | non- | At1g61950 | leucine | ARA_LEAF | p-PcUBI | GC | 99 | 169 |
| | targeted | | | | | | | |
| 18869 | non- | At1g61950 | valine | ARA_LEAF | p-PcUBI | GC | 43 | 84 |
| | targeted | | | | | | | |
| 102119 | non- | At1g68410 | isoleucine | ARA_LEAF | p-PcUBI | GC | 41 | 140 |
| | targeted | | | | | | | |
| 102119 | non- | At1g68410 | leucine | ARA_LEAF | p-PcUBI | GC | 50 | 137 |
| | targeted | | | | | | | |
| 19419 | non- | At1g72770 | valine | ARA_LEAF | p-PcUBI | GC | 22 | 134 |
| | targeted | | | | | | | |
| 19502 | non- | At2g17560 | valine | ARA_LEAF | p-PcUBI | GC | 25 | 108 |
| | targeted | | | | | | | |
| 19874 | non- | At2g28890 | isoleucine | ARA_LEAF | p-PcUBI | GC | 33 | 738 |
| | targeted | | | | | | | |
| 19874 | non- | At2g28890 | valine | ARA_LEAF | p-PcUBI | GC | 26 | 321 |
| | targeted | | | | | | | |
| 19919 | non- | At2g30360 | isoleucine | ARA_LEAF | p-PcUBI | GC | 35 | 810 |
| | targeted | | | | | | | |
| 19919 | non- | At2g30360 | leucine | ARA_LEAF | p-PcUBI | GC | 64 | 947 |
| | targeted | | | | | | | |
| 19919 | non- | At2g30360 | valine | ARA_LEAF | p-PcUBI | GC | 137 | 277 |
| | targeted | | | | | | | |
| 20346 | non- | At2g30540 | isoleucine | ARA_LEAF | p-PcUBI | GC | 39 | 206 |
| | targeted | | | | | | | |
| 20346 | non- | At2g30540 | leucine | ARA_LEAF | p-PcUBI | GC | 66 | 243 |
| | targeted | | | | | | | |
| 20346 | non- | At2g30540 | valine | ARA_LEAF | p-PcUBI | GC | 24 | 183 |
| | targeted | | | | | | | |
| 21159 | non- | At2g47880 | valine | ARA_LEAF | p-PcUBI | GC | 65 | 227 |
| | targeted | | | | | | | |
| 21497 | plastidic | At3g04050 | isoleucine | ARA_LEAF | p-PcUBI | GC | 39 | 470 |
| 21497 | plastidic | At3g04050 | leucine | ARA_LEAF | p-PcUBI | GC | 64 | 1234 |
| 21497 | plastidic | At3g04050 | valine | ARA_LEAF | p-PcUBI | GC | 35 | 681 |
| 22249 | non- | At3g08710 | isoleucine | ARA_LEAF | p-PcUBI | GC | 34 | 264 |
| | targeted | | | | | | | |
| 22249 | non- | At3g08710 | leucine | ARA_LEAF | p-PcUBI | GC | 53 | 221 |
| | targeted | | | | | | | |
| 22611 | non- | At3g11650 | isoleucine | ARA_LEAF | p-PcUBI | GC | 50 | 191 |
| | targeted | | | | | | | |
| 22611 | non- | At3g11650 | leucine | ARA_LEAF | p-PcUBI | GC | 52 | 216 |
| | targeted | | | | | | | |
| 22832 | non- | At3g18524 | isoleucine | ARA_LEAF | p-PcUBI | GC | 34 | 61 |
| | targeted | | | | | | | |
| 22832 | non- | At3g18524 | leucine | ARA_LEAF | p-PcUBI | GC | 61 | 87 |
| | targeted | | | | | | | |
| 22832 | non- | At3g18524 | valine | ARA_LEAF | p-PcUBI | GC | 21 | 31 |
| | targeted | | | | | | | |
| 1815 | non- | At3g23000 | isoleucine | ARA_LEAF | p-PcUBI | GC | 63 | 717 |
| | targeted | | | | | | | |
| 1815 | non- | At3g23000 | leucine | ARA_LEAF | p-PcUBI | GC | 82 | 562 |
| | targeted | | | | | | | |
| 1815 | non- | At3g23000 | valine | ARA_LEAF | p-PcUBI | GC | 42 | 249 |
| | targeted | | | | | | | |
| 68727 | non- | At3g26400 | isoleucine | ARA_LEAF | p-PcUBI | GC | 46 | 133 |
| | targeted | | | | | | | |
| 68727 | non- | At3g26400 | leucine | ARA_LEAF | p-PcUBI | GC | 51 | 129 |
| | targeted | | | | | | | |
| 68727 | non- | At3g26400 | valine | ARA_LEAF | p-PcUBI | GC | 32 | 76 |
| | targeted | | | | | | | |
| 68777 | non- | At3g27540 | isoleucine | ARA_LEAF | p-PcUBI | GC | 79 | 428 |
| | targeted | | | | | | | |
| 68777 | non- | At3g27540 | leucine | ARA_LEAF | p-PcUBI | GC | 89 | 469 |
| | targeted | | | | | | | |
| 68777 | non- | At3g27540 | valine | ARA_LEAF | p-PcUBI | GC | 26 | 161 |
| | targeted | | | | | | | |
| 2367 | non- | At3g61830 | isoleucine | ARA_LEAF | p-PcUBI | GC | 39 | 965 |
| | targeted | | | | | | | |
| 2367 | non- | At3g61830 | leucine | ARA_LEAF | p-PcUBI | GC | 67 | 1444 |
| | targeted | | | | | | | |
| 2367 | non- | At3g61830 | valine | ARA_LEAF | p-PcUBI | GC | 27 | 397 |
| | targeted | | | | | | | |
| 2935 | non- | At3g62950 | isoleucine | ARA_LEAF | p-PcUBI | GC | 60 | 274 |
| | targeted | | | | | | | |
| 2935 | non- | At3g62950 | leucine | ARA_LEAF | p-PcUBI | GC | 85 | 337 |
| | targeted | | | | | | | |
| 2935 | non- | At3g62950 | valine | ARA_LEAF | p-PcUBI | GC | 37 | 118 |
| | targeted | | | | | | | |
| 24232 | non- | At4g18880 | isoleucine | ARA_LEAF | p-PcUBI | GC | 52 | 136 |
| | targeted | | | | | | | |
| 68849 | non- | At4g26080 | valine | ARA_LEAF | p-PcUBI | GC | 21 | 56 |
| | targeted | | | | | | | |
| 4040 | non- | At4g32480 | isoleucine | ARA_LEAF | p-PcUBI | GC | 74 | 355 |
| | targeted | | | | | | | |
| 4040 | non- | At4g32480 | leucine | ARA_LEAF | p-PcUBI | GC | 53 | 320 |
| | targeted | | | | | | | |
| 4040 | non- | At4g32480 | valine | ARA_LEAF | p-PcUBI | GC | 29 | 286 |
| | targeted | | | | | | | |
| 24311 | non- | At4g34160 | isoleucine | ARA_LEAF | p-PcUBI | GC | 46 | 199 |
| | targeted | | | | | | | |
| 24311 | non- | At4g34160 | leucine | ARA_LEAF | p-PcUBI | GC | 83 | 188 |
| | targeted | | | | | | | |
| 24311 | non- | At4g34160 | valine | ARA_LEAF | p-PcUBI | GC | 23 | 115 |
| | targeted | | | | | | | |
| 4348 | non- | At4g35310 | isoleucine | ARA_LEAF | p-PcUBI | GC | 35 | 645 |
| | targeted | | | | | | | |
| 4348 | non- | At4g35310 | leucine | ARA_LEAF | p-PcUBI | GC | 164 | 698 |
| | targeted | | | | | | | |
| 4348 | non- | At4g35310 | valine | ARA_LEAF | p-PcUBI | GC | 39 | 329 |
| | targeted | | | | | | | |
| 68987 | non- | At5g02760 | isoleucine | ARA_LEAF | p-PcUBI | GC | 40 | 305 |
| | targeted | | | | | | | |
| 25283 | non- | At5g16650 | isoleucine | ARA_LEAF | p-PcUBI | GC | 48 | 142 |
| | targeted | | | | | | | |
| 25283 | non- | At5g16650 | valine | ARA_LEAF | p-PcUBI | GC | 24 | 86 |
| | targeted | | | | | | | |
| 4904 | non- | At5g18600 | valine | ARA_LEAF | p-PcUBI | GC | 31 | 59 |
| | targeted | | | | | | | |
| 69574 | non- | At5g39950 | leucine | ARA_LEAF | p-PcUBI | GC | 53 | 145 |
| | targeted | | | | | | | |
| 69574 | non- | At5g39950 | valine | ARA_LEAF | p-PcUBI | GC | 23 | 50 |
| | targeted | | | | | | | |
| 102223 | non- | At5g45060 | valine | ARA_LEAF | p-PcUBI | GC | 24 | 70 |
| | targeted | | | | | | | |
| 102373 | plastidic | At5g56350 | isoleucine | ARA_LEAF | p-PcUBI | GC | 35 | 84 |
| 102373 | plastidic | At5g56350 | leucine | ARA_LEAF | p-PcUBI | GC | 56 | 69 |
| 70006 | non- | At5g60110 | valine | ARA_LEAF | p-PcUBI | GC | 21 | 41 |
| | targeted | | | | | | | |
| 102899 | non- | At5g60440 | isoleucine | ARA_LEAF | p-PcUBI | GC | 33 | 474 |
| | targeted | | | | | | | |
| 102899 | non- | At5g60440 | leucine | ARA_LEAF | p-PcUBI | GC | 51 | 643 |
| | targeted | | | | | | | |
| 70038 | plastidic | At5g63680 | isoleucine | ARA_LEAF | p-PcUBI | GC | 41 | 182 |
| 70038 | plastidic | At5g63680 | leucine | ARA_LEAF | p-PcUBI | GC | 55 | 358 |
| 70038 | plastidic | At5g63680 | valine | ARA_LEAF | p-PcUBI | GC | 31 | 254 |
| 5493 | non- | At5g64920 | valine | ARA_LEAF | p-PcUBI | GC | 35 | 83 |
| | targeted | | | | | | | |
| 5557 | non- | Avin-DRAFT_1495 | isoleucine | ARA_LEAF | p-PcUBI | GC | 85 | 297 |
| | targeted | | | | | | | |
| 5557 | non- | AvinDRAFT_1495 | leucine | ARA_LEAF | p-PcUBI | GC | 98 | 461 |
| | targeted | | | | | | | |
| 5557 | non- | AvinDRAFT_1495 | valine | ARA_LEAF | p-PcUBI | GC | 50 | 122 |
| | targeted | | | | | | | |
| 26434 | non- | AvinDRAFT_1806 | valine | ARA_LEAF | p-PcUBI | GC | 21 | 47 |
| | targeted | | | | | | | |
| 6040 | non- | AvinDRAFT_2091 | valine | ARA_LEAF | p-PcUBI | GC | 32 | 131 |
| | targeted | | | | | | | |
| 27021 | non- | AvinDRAFT_2344 | isoleucine | ARA_LEAF | p-PcUBI | GC | 43 | 379 |
| | targeted | | | | | | | |
| 27021 | non- | AvinDRAFT_2344 | leucine | ARA_LEAF | p-PcUBI | GC | 114 | 353 |
| | targeted | | | | | | | |
| 27021 | non- | AvinDRAFT_2344 | valine | ARA_LEAF | p-PcUBI | GC | 47 | 203 |
| | targeted | | | | | | | |
| 71763 | non- | Avin- | valine | ARA_LEAF | p-PcUBI | GC | 22 | 62 |
| | targeted | DRAFT_2369 | | | | | | |
| 27882 | non- | AvinDRAFT_2521 | valine | ARA_LEAF | p-PcUBI | GC | 31 | 94 |
| | targeted | | | | | | | |
| 28738 | non- | AvinDRAFT_3159 | isoleucine | ARA_LEAF | p-PcUBI | GC | 114 | 177 |
| | targeted | | | | | | | |
| 28738 | non- | AvinDRAFT_3159 | leucine | ARA_LEAF | p-PcUBI | GC | 134 | 194 |
| | targeted | | | | | | | |
| 28738 | non- | AvinDRAFT_3159 | valine | ARA_LEAF | p-PcUBI | GC | 46 | 98 |
| | targeted | | | | | | | |
| 29286 | non- | AvinDRAFT_3209 | isoleucine | ARA_LEAF | p-PcUBI | GC | 41 | 207 |
| | targeted | | | | | | | |
| 29286 | non- | AvinDRAFT_3209 | leucine | ARA_LEAF | p-PcUBI | GC | 75 | 190 |
| | targeted | | | | | | | |
| 29286 | non- | AvinDRAFT_3209 | valine | ARA_LEAF | p-PcUBI | GC | 37 | 99 |
| | targeted | | | | | | | |
| 29397 | non- | AvinDRAFT_3250 | isoleucine | ARA_LEAF | p-PcUBI | GC | 120 | 251 |
| | targeted | | | | | | | |
| 29397 | non- | AvinDRAFT_3250 | leucine | ARA_LEAF | p-PcUBI | GC | 85 | 298 |
| | targeted | | | | | | | |
| 29397 | non- | AvinDRAFT_3250 | valine | ARA_LEAF | p-PcUBI | GC | 25 | 101 |
| | targeted | | | | | | | |
| 29500 | non- | AvinDRAFT_3253 | isoleucine | ARA_LEAF | p-PcUBI | GC | 35 | 67 |
| | targeted | | | | | | | |
| 29500 | non- | AvinDRAFT_3253 | leucine | ARA_LEAF | p-PcUBI | GC | 51 | 66 |
| | targeted | | | | | | | |
| 29500 | non- | AvinDRAFT_3253 | valine | ARA_LEAF | p-PcUBI | GC | 20 | 52 |
| | targeted | | | | | | | |
| 72520 | non- | AVIN- | valine | ARA_LEAF | p-PcUBI | GC | 22 | 64 |
| | targeted | DRAFT_4128 | | | | | | |
| 31717 | non- | AvinDRAFT_4384 | valine | ARA_LEAF | p-PcUBI | GC | 24 | 32 |
| | targeted | | | | | | | |
| 31926 | non- | AvinDRAFT_4562 | isoleucine | ARA_LEAF | p-PcUBI | GC | 59 | 77 |
| | targeted | | | | | | | |
| 31926 | non- | AvinDRAFT_4562 | valine | ARA_LEAF | p-PcUBI | GC | 26 | 56 |
| | targeted | | | | | | | |
| 73719 | non- | AvinDRAFT_4606 | isoleucine | ARA_LEAF | p-PcUBI | GC | 40 | 127 |
| | targeted | | | | | | | |
| 73719 | non- | AvinDRAFT_4606 | leucine | ARA_LEAF | p-PcUBI | GC | 58 | 85 |
| | targeted | | | | | | | |
| 74163 | non- | AvinDRAFT_4836 | isoleucine | ARA_LEAF | p-PcUBI | GC | 64 | 213 |
| | targeted | | | | | | | |
| 74163 | non- | AvinDRAFT_4836 | leucine | ARA_LEAF | p-PcUBI | GC | 68 | 176 |
| | targeted | | | | | | | |
| 74163 | non- | AvinDRAFT_4836 | valine | ARA_LEAF | p-PcUBI | GC | 24 | 135 |
| | targeted | | | | | | | |
| 102941 | non- | AvinDRAFT_4847 | valine | ARA_LEAF | p-PcUBI | GC | 26 | 31 |
| | targeted | | | | | | | |
| 6510 | non- | AvinDRAFT_5103 | isoleucine | ARA_LEAF | p-PcUBI | GC | 41 | 2397 |
| | targeted | | | | | | | |
| 6510 | non- | AvinDRAFT_5103 | leucine | ARA_LEAF | p-PcUBI | GC | 51 | 2753 |
| | targeted | | | | | | | |
| 6510 | non- | AvinDRAFT_5103 | valine | ARA_LEAF | p-PcUBI | GC | 24 | 951 |
| | targeted | | | | | | | |
| 32308 | non- | AvinDRAFT_5292 | isoleucine | ARA_LEAF | p-PcUBI | GC | 335 | 446 |
| | targeted | | | | | | | |
| 32308 | non- | AvinDRAFT_5292 | leucine | ARA_LEAF | p-PcUBI | GC | 469 | 550 |
| | targeted | | | | | | | |
| 32308 | non- | AvinDRAFT_5292 | valine | ARA_LEAF | p-PcUBI | GC | 132 | 187 |
| | targeted | | | | | | | |
| 74662 | non- | AvinDRAFT_5579 | leucine | ARA_LEAF | p-PcUBI | GC | 57 | 94 |
| | targeted | | | | | | | |
| 33596 | non- | AvinDRAFT_6093 | isoleucine | ARA_LEAF | p-PcUBI | GC | 44 | 134 |
| | targeted | | | | | | | |
| 34044 | non- | AvinDRAFT_6700 | isoleucine | ARA_LEAF | p-PcUBI | GC | 32 | 34 |
| | targeted | | | | | | | |
| 34044 | non- | AvinDRAFT_6700 | leucine | ARA_LEAF | p-PcUBI | GC | 31 | 53 |
| | targeted | | | | | | | |
| 34044 | non- | AvinDRAFT_6700 | valine | ARA_LEAF | p-PcUBI | GC | 22 | 29 |
| | targeted | | | | | | | |
| 103433 | non- | AY623894 | isoleucine | ARA_LEAF | p-PcUBI | GC | 49 | 103 |
| | targeted | | | | | | | |
| 103433 | non- | AY623894 | valine | ARA_LEAF | p-PcUBI | GC | 30 | 46 |
| | targeted | | | | | | | |
| 34889 | plastidic | B0004 | isoleucine | ARA_LEAF | p-Super | GC | 40 | 468 |
| 34889 | plastidic | B0004 | leucine | ARA_LEAF | p-Super | GC | 96 | 297 |
| 34889 | plastidic | B0004 | valine | ARA_LEAF | p-Super | GC | 33 | 220 |
| 103958 | non- | B0050 | valine | ARA_LEAF | p-Super | GC | 24 | 122 |
| | targeted | | | | | | | |
| 6818 | non- | B0078 | leucine | ARA_SEED_2 | p-USP | GC | 44 | 73 |
| | targeted | | | | | | | |
| 35733 | plastidic | B0344 | leucine | ARA_SEED_2 | p-USP | LC | 224 | 288 |
| 7269 | plastidic | B0348 | isoleucine | ARA_SEED_2 | p-USP | GC | 64 | 316 |
| 7269 | plastidic | B0348 | valine | ARA_SEED_2 | p-USP | GC | 35 | 236 |
| 7333 | non- | B0449 | isoleucine | ARA_LEAF | p-Super | GC | 75 | 2019 |
| | targeted | | | | | | | |
| 7333 | non- | B0449 | leucine | ARA_LEAF | p-Super | GC | 82 | 2204 |
| | targeted | | | | | | | |
| 7333 | non- | B0449 | valine | ARA_LEAF | p-Super | GC | 21 | 774 |
| | targeted | | | | | | | |
| 7686 | non- | B0486 | isoleucine | ARA_LEAF | p-Super | GC | 68 | 394 |
| | targeted | | | | | | | |
| 35967 | plastidic | B0593 | isoleucine | ARA_LEAF | p-Super | GC | 77 | 474 |
| 35967 | plastidic | B0593 | leucine | ARA_LEAF | p-Super | GC | 74 | 478 |
| 35967 | plastidic | B0593 | valine | ARA_LEAF | p-Super | GC | 62 | 214 |
| 36114 | non- | B0752 | isoleucine | ARA_LEAF | p-Super | GC | 36 | 58 |
| | targeted | | | | | | | |
| 36114 | non- | B0752 | leucine | ARA_LEAF | p-Super | GC | 54 | 81 |
| | targeted | | | | | | | |
| 7917 | non- | B0898 | isoleucine | ARA_LEAF | p-Super | GC | 40 | 6183 |
| | targeted | | | | | | | |
| 7917 | non- | B0898 | leucine | ARA_LEAF | p-Super | GC | 59 | 5609 |
| | targeted | | | | | | | |
| 7917 | non- | B0898 | valine | ARA_LEAF | p-Super | GC | 23 | 2254 |
| | targeted | | | | | | | |
| 36623 | non- | B0962 | leucine | ARA_SEED_2 | p-USP | GC | 52 | 274 |
| | targeted | | | | | | | |
| 36670 | non- | B0963 | valine | ARA_LEAF | p-Super | GC | 34 | 127 |
| | targeted | | | | | | | |
| 36809 | non- | B0980 | valine | ARA_SEED_2 | p-USP | GC | 33 | 99 |
| | targeted | | | | | | | |
| 7941 | non- | B1003 | isoleucine | ARA_LEAF | p-Super | GC | 185 | 1202 |
| | targeted | | | | | | | |
| 7941 | non- | B1003 | leucine | ARA_LEAF | p-Super | GC | 183 | 1058 |
| | targeted | | | | | | | |
| 7941 | non- | B1003 | valine | ARA_LEAF | p-Super | GC | 71 | 549 |
| | targeted | | | | | | | |
| 37394 | non- | B1163 | isoleucine | ARA_LEAF | p-Super | GC | 33 | 107 |
| | targeted | | | | | | | |
| 37394 | non- | B1163 | leucine | ARA_LEAF | p-Super | GC | 53 | 134 |
| | targeted | | | | | | | |
| 37394 | non- | B1163 | valine | ARA_LEAF | p-Super | GC | 27 | 47 |
| | targeted | | | | | | | |
| 104134 | plastidic | B1184 | leucine | ARA_SEED_2 | p-Super | GC | 31 | 169 |
| 104134 | plastidic | B1184 | valine | ARA_SEED_2 | p-Super | GC | 30 | 30 |
| 37483 | non- | B1255 | isoleucine | ARA_LEAF | p-Super | GC | 45 | 155 |
| | targeted | | | | | | | |
| 37483 | non- | B1255 | leucine | ARA_LEAF | p-Super | GC | 54 | 178 |
| | targeted | | | | | | | |
| 37483 | non- | B1255 | valine | ARA_LEAF | p-Super | GC | 20 | 69 |
| | targeted | | | | | | | |
| 37539 | plastidic | B1263 | isoleucine | ARA_SEED_2 | p-USP | GC | 22 | 106 |
| 37539 | plastidic | B1263 | leucine | ARA_SEED_2 | p-USP | GC | 58 | 74 |
| 37539 | plastidic | B1263 | valine | ARA_SEED_2 | p-USP | GC | 29 | 83 |
| 37658 | plastidic | B1297 | leucine | ARA_SEED_2 | p-USP | LC | 195 | 355 |
| 37807 | non- | B1300 | valine | ARA_LEAF | p-Super | GC | 20 | 52 |
| | targeted | | | | | | | |
| 38289 | non- | B1445 | valine | ARA_LEAF | p-Super | GC | 20 | 49 |
| | targeted | | | | | | | |
| 77774 | non- | B1479 | valine | ARA_SEED_2 | p-USP | GC | 40 | 188 |
| | targeted | | | | | | | |
| 7947 | non- | B1522 | isoleucine | ARA_LEAF | p-Super | GC | 41 | 920 |
| | targeted | | | | | | | |
| 7947 | non- | B1522 | leucine | ARA_LEAF | p-Super | GC | 213 | 780 |
| | targeted | | | | | | | |
| 7947 | non- | B1522 | valine | ARA_LEAF | p-Super | GC | 20 | 336 |
| | targeted | | | | | | | |
| 38300 | non- | B1597 | isoleucine | ARA_LEAF | p-Super | GC | 69 | 218 |
| | targeted | | | | | | | |
| 38300 | non- | B1597 | leucine | ARA_LEAF | p-Super | GC | 75 | 252 |
| | targeted | | | | | | | |
| 38300 | non- | B1597 | valine | ARA_LEAF | p-Super | GC | 44 | 105 |
| | targeted | | | | | | | |
| 7992 | non- | B1601 | valine | ARA_LEAF | p-Super | GC | 21 | 42 |
| | targeted | | | | | | | |
| 78753 | non- | B1670 | valine | ARA_LEAF | p-Super | GC | 20 | 80 |
| | targeted | | | | | | | |
| 78771 | non- | B1672 | valine | ARA_LEAF | p-Super | GC | 23 | 90 |
| | targeted | | | | | | | |
| 8363 | plastidic | B1854 | leucine | ARA_SEED_2 | p-USP | GC | 58 | 227 |
| 78970 | non- | B1873 | valine | ARA_LEAF | p-Super | GC | 24 | 49 |
| | targeted | | | | | | | |
| 79056 | non- | B1897 | isoleucine | ARA_LEAF | p-Super | GC | 41 | 333 |
| | targeted | | | | | | | |
| 79056 | non- | B1897 | leucine | ARA_LEAF | p-Super | GC | 154 | 467 |
| | targeted | | | | | | | |
| 79056 | non- | B1897 | valine | ARA_LEAF | p-Super | GC | 38 | 159 |
| | targeted | | | | | | | |
| 79217 | non- | B2027 | isoleucine | ARA_LEAF | p-Super | GC | 40 | 1182 |
| | targeted | | | | | | | |
| 79217 | non- | B2027 | leucine | ARA_LEAF | p-Super | GC | 68 | 1185 |
| | targeted | | | | | | | |
| 104277 | non- | B2053 | valine | ARA_LEAF | p-Super | GC | 22 | 50 |
| | targeted | | | | | | | |
| 38947 | non- | B2063 | isoleucine | ARA_LEAF | p-Super | GC | 35 | 778 |
| | targeted | | | | | | | |
| 38947 | non- | B2063 | leucine | ARA_LEAF | p-Super | GC | 62 | 415 |
| | targeted | | | | | | | |
| 104616 | non- | B2291 | valine | ARA_LEAF | p-Super | GC | 21 | 41 |
| | targeted | | | | | | | |
| 39255 | non- | B2405 | isoleucine | ARA_LEAF | p-Super | GC | 54 | 74 |
| | targeted | | | | | | | |
| 39255 | non- | B2405 | valine | ARA_LEAF | p-Super | GC | 24 | 74 |
| | targeted | | | | | | | |
| 9167 | non- | B2513 | isoleucine | ARA_LEAF | p-Super | GC | 63 | 339 |
| | targeted | | | | | | | |
| 9167 | non- | B2513 | leucine | ARA_LEAF | p-Super | GC | 60 | 464 |
| | targeted | | | | | | | |
| 9167 | non- | B2513 | valine | ARA_LEAF | p-Super | GC | 23 | 140 |
| | targeted | | | | | | | |
| 79627 | non- | B2551 | isoleucine | ARA_SEED_2 | p-USP | GC | 24 | 481 |
| | targeted | | | | | | | |
| 40665 | non- | B2613 | valine | ARA_LEAF | p-Super | GC | 24 | 29 |
| | targeted | | | | | | | |
| 40726 | non- | B2634 | isoleucine | ARA_LEAF | p-Super | GC | 34 | 118 |
| | targeted | | | | | | | |
| 40726 | non- | B2634 | leucine | ARA_LEAF | p-Super | GC | 52 | 124 |
| | targeted | | | | | | | |
| 9244 | non- | B2673 | isoleucine | ARA_LEAF | p-Super | GC | 44 | 144 |
| | targeted | | | | | | | |
| 80756 | non- | B2701 | isoleucine | ARA_LEAF | p-Super | GC | 57 | 501 |
| | targeted | | | | | | | |
| 80756 | non- | B2701 | leucine | ARA_LEAF | p-Super | GC | 348 | 403 |
| | targeted | | | | | | | |
| 80756 | non- | B2701 | valine | ARA_LEAF | p-Super | GC | 26 | 218 |
| | targeted | | | | | | | |
| 40741 | non- | B2714 | valine | ARA_LEAF | p-Super | GC | 21 | 38 |
| | targeted | | | | | | | |
| 80906 | non- | B2739 | isoleucine | ARA_LEAF | p-Super | GC | 51 | 542 |
| | targeted | | | | | | | |
| 80906 | non- | B2739 | leucine | ARA_LEAF | p-Super | GC | 57 | 800 |
| | targeted | | | | | | | |
| 80906 | non- | B2739 | valine | ARA_LEAF | p-Super | GC | 70 | 169 |
| | targeted | | | | | | | |
| 40795 | non- | B2812 | isoleucine | ARA_LEAF | p-Super | GC | 50 | 171 |
| | targeted | | | | | | | |
| 40795 | non- | B2812 | leucine | ARA_LEAF | p-Super | GC | 59 | 203 |
| | targeted | | | | | | | |
| 81935 | non- | B2839 | isoleucine | ARA_LEAF | p-Super | GC | 35 | 190 |
| | targeted | | | | | | | |
| 81935 | non- | B2839 | leucine | ARA_LEAF | p-Super | GC | 118 | 154 |
| | targeted | | | | | | | |
| 81935 | non- | B2839 | valine | ARA_LEAF | p-Super | GC | 30 | 94 |
| | targeted | | | | | | | |
| 41073 | non- | B2914 | isoleucine | ARA_SEED_2 | p-USP | GC | 30 | 97 |
| | targeted | | | | | | | |
| 9333 | non- | B2923 | valine | ARA_LEAF | p-Super | GC | 23 | 27 |
| | targeted | | | | | | | |
| 42046 | non- | B3064 | isoleucine | ARA_SEED_2 | p-Super | GC | 25 | 100 |
| | targeted | | | | | | | |
| 42046 | non- | B3064 | leucine | ARA_SEED_2 | p-Super | GC | 32 | 127 |
| | targeted | | | | | | | |
| 42046 | non- | B3064 | valine | ARA_SEED_2 | p-Super | GC | 32 | 82 |
| | targeted | | | | | | | |
| 82424 | non- | B3083 | isoleucine | ARA_LEAF | p-Super | GC | 40 | 74 |
| | targeted | | | | | | | |
| 82424 | non- | B3083 | valine | ARA_LEAF | p-Super | GC | 22 | 48 |
| | targeted | | | | | | | |
| 104660 | plastidic | B3112 | leucine | ARA_SEED_2 | p-USP | GC | 49 | 78 |
| 104871 | plastidic | B3117 | valine | ARA_SEED_2 | p-USP | GC | 29 | 130 |
| 9492 | non- | B3256 | leucine | ARA_SEED_2 | p-USP | GC | 42 | 465 |
| | targeted | | | | | | | |
| 82572 | non- | B3352 | valine | ARA_LEAF | p-Super | GC | 21 | 44 |
| | targeted | | | | | | | |
| 94051 | mitochondrial | B3429 | isoleucine | ARA_LEAF | p-Super | GC | 73 | 242 |
| 94051 | mitochondrial | B3429 | leucine | ARA_LEAF | p-Super | GC | 53 | 292 |
| 105332 | non- | B3490 | valine | ARA_LEAF | p-Super | GC | 21 | 44 |
| | targeted | | | | | | | |
| 10172 | plastidic | B3572 | isoleucine | ARA_SEED_2 | p-USP | GC | 23 | 132 |
| 10172 | plastidic | B3572 | leucine | ARA_SEED_2 | p-USP | GC | 32 | 115 |
| 42931 | non- | B3634 | isoleucine | ARA_LEAF | p-Super | GC | 42 | 126 |
| | targeted | | | | | | | |
| 42931 | non- | B3634 | leucine | ARA_LEAF | p-Super | GC | 94 | 289 |
| | targeted | | | | | | | |
| 42931 | non- | B3634 | valine | ARA_LEAF | p-Super | GC | 20 | 99 |
| | targeted | | | | | | | |
| 83629 | plastidic | B3670 | leucine | ARA_SEED_2 | p-USP | GC | 58 | 77 |
| 105354 | plastidic | B3770 | isoleucine | ARA_LEAF | p-Super | GC | 39 | 57 |
| 10252 | non- | B3771 | isoleucine | ARA_SEED_2 | p-USP | GC | 23 | 173 |
| | targeted | | | | | | | |
| 10252 | non- | B3771 | leucine | ARA_SEED_2 | p-USP | GC | 43 | 176 |
| | targeted | | | | | | | |
| 10252 | plastidic | B3771 | leucine | ARA_SEED_2 | p-USP | GC | 42 | 49 |
| 10252 | non- | B3771 | valine | ARA_SEED_2 | p-USP | GC | 37 | 128 |
| | targeted | | | | | | | |
| 94386 | non- | B3774 | isoleucine | ARA_LEAF | p-Super | GC | 55 | 61 |
| | targeted | | | | | | | |
| 94386 | plastidic | B3774 | leucine | ARA_SEED_2 | p-USP | GC | 38 | 127 |
| 43839 | non- | B3813 | isoleucine | ARA_SEED_2 | p-USP | GC | 33 | 137 |
| | targeted | | | | | | | |
| 43839 | non- | B3813 | leucine | ARA_SEED_2 | p-USP | GC | 40 | 118 |
| | targeted | | | | | | | |
| 43839 | non- | B3813 | valine | ARA_SEED_2 | p-USP | GC | 27 | 94 |
| | targeted | | | | | | | |
| 105602 | plastidic | B3850 | leucine | ARA_SEED_2 | p-USP | LC | 181 | 667 |
| 105659 | plastidic | B3908 | leucine | ARA_SEED_2 | p-USP | LC | 164 | 466 |
| 44372 | non- | B3989 | isoleucine | ARA_LEAF | p-Super | GC | 34 | 1033 |
| | targeted | | | | | | | |
| 44372 | non- | B3989 | leucine | ARA_LEAF | p-Super | GC | 53 | 1312 |
| | targeted | | | | | | | |
| 44372 | non- | B3989 | valine | ARA_LEAF | p-Super | GC | 23 | 332 |
| | targeted | | | | | | | |
| 10740 | non- | B4029 | isoleucine | ARA_LEAF | p-Super | GC | 39 | 2813 |
| | targeted | | | | | | | |
| 10740 | non- | B4029 | leucine | ARA_LEAF | p-Super | GC | 54 | 3551 |
| | targeted | | | | | | | |
| 10740 | non- | B4029 | valine | ARA_LEAF | p-Super | GC | 28 | 1098 |
| | targeted | | | | | | | |
| 106532 | non- | B4050 | isoleucine | ARA_LEAF | p-Super | GC | 105 | 106 |
| | targeted | | | | | | | |
| 94542 | non- | B4056 | isoleucine | ARA_LEAF | p-Super | GC | 39 | 51 |
| | targeted | | | | | | | |
| 94542 | non- | B4056 | leucine | ARA_LEAF | p-Super | GC | 44 | 52 |
| | targeted | | | | | | | |
| 45321 | non- | B4256 | isoleucine | ARA_LEAF | p-Super | GC | 51 | 240 |
| | targeted | | | | | | | |
| 45321 | non- | B4256 | leucine | ARA_LEAF | p-Super | GC | 194 | 267 |
| | targeted | | | | | | | |
| 45321 | non- | B4256 | valine | ARA_LEAF | p-Super | GC | 53 | 137 |
| | targeted | | | | | | | |
| 106538 | plastidic | B4263 | valine | ARA_LEAF | p-Super | GC | 23 | 38 |
| 45394 | non- | B4321 | isoleucine | ARA_LEAF | p-Super | GC | 43 | 228 |
| | targeted | | | | | | | |
| 45394 | non- | B4321 | leucine | ARA_LEAF | p-Super | GC | 113 | 284 |
| | targeted | | | | | | | |
| 45394 | non- | B4321 | valine | ARA_LEAF | p-Super | GC | 20 | 85 |
| | targeted | | | | | | | |
| 45556 | non- | B4384 | isoleucine | ARA_SEED_2 | p-USP | GC | 64 | 169 |
| | targeted | | | | | | | |
| 45757 | non- | C_pp004096192r | isoleucine | ARA_LEAF | p-Super | GC | 85 | 176 |
| | targeted | | | | | | | |
| 45757 | non- | C_pp004096192r | leucine | ARA_LEAF | p-Super | GC | 65 | 147 |
| | targeted | | | | | | | |
| 45757 | non- | C_pp004096192r | valine | ARA_LEAF | p-Super | GC | 21 | 74 |
| | targeted | | | | | | | |
| 45795 | non- | GM02LC11114 | isoleucine | ARA_LEAF | p-PcUBI | GC | 64 | 105 |
| | targeted | | | | | | | |
| 45795 | non- | GM02LC11114 | leucine | ARA_LEAF | p-PcUBI | GC | 62 | 113 |
| | targeted | | | | | | | |
| 10811 | non- | GM02LC12622 | isoleucine | ARA_LEAF | p-PcUBI | GC | 43 | 472 |
| | targeted | | | | | | | |
| 10811 | non- | GM02LC12622 | leucine | ARA_LEAF | p-PcUBI | GC | 63 | 562 |
| | targeted | | | | | | | |
| 10811 | non- | GM02LC12622 | valine | ARA_LEAF | p-PcUBI | GC | 24 | 155 |
| | targeted | | | | | | | |
| 45897 | non- | GM02LC15313 | isoleucine | ARA_LEAF | p-PcUBI | GC | 53 | 172 |
| | targeted | | | | | | | |
| 45897 | non- | GM02LC15313 | leucine | ARA_LEAF | p-PcUBI | GC | 80 | 144 |
| | targeted | | | | | | | |
| 45897 | non- | GM02LC15313 | valine | ARA_LEAF | p-PcUBI | GC | 30 | 99 |
| | targeted | | | | | | | |
| 106688 | non- | GM02LC17384 | valine | ARA_LEAF | p-PcUBI | GC | 21 | 63 |
| | targeted | | | | | | | |
| 46515 | non- | GM02LC17556 | isoleucine | ARA_LEAF | p-PcUBI | GC | 51 | 366 |
| | targeted | | | | | | | |
| 46515 | non- | GM02LC17556 | leucine | ARA_LEAF | p-PcUBI | GC | 55 | 406 |
| | targeted | | | | | | | |
| 46515 | non- | GM02LC17556 | valine | ARA_LEAF | p-PcUBI | GC | 21 | 90 |
| | targeted | | | | | | | |
| 84198 | non- | GM02LC21368 | isoleucine | ARA_LEAF | p-PcUBI | GC | 54 | 890 |
| | targeted | | | | | | | |
| 84198 | non- | GM02LC21368 | leucine | ARA_LEAF | p-PcUBI | GC | 100 | 811 |
| | targeted | | | | | | | |
| 84198 | non- | GM02LC21368 | valine | ARA_LEAF | p-PcUBI | GC | 21 | 230 |
| | targeted | | | | | | | |
| 46850 | non- | GM02LC44512 | isoleucine | ARA_LEAF | p-PcUBI | GC | 40 | 128 |
| | targeted | | | | | | | |
| 46850 | non- | GM02LC44512 | leucine | ARA_LEAF | p-PcUBI | GC | 82 | 122 |
| | targeted | | | | | | | |
| 46850 | non- | GM02LC44512 | valine | ARA_LEAF | p-PcUBI | GC | 22 | 86 |
| | targeted | | | | | | | |
| 47076 | non- | GM02LC5744 | leucine | ARA_LEAF | p-PcUBI | GC | 53 | 754 |
| | targeted | | | | | | | |
| 84265 | plastidic | Sll0228 | valine | ARA_LEAF | p-PcUBI | GC | 21 | 33 |
| 84441 | plastidic | Sll0240 | valine | ARA_LEAF | p-PcUBI | GC | 24 | 32 |
| 49143 | plastidic | Sll0248 | isoleucine | ARA_LEAF | p-PcUBI | GC | 53 | 313 |
| 49143 | plastidic | Sll0248 | leucine | ARA_LEAF | p-PcUBI | GC | 70 | 203 |
| 49828 | non- | Sll0290 | isoleucine | ARA_LEAF | p-PcUBI | GC | 35 | 238 |
| | targeted | | | | | | | |
| 49828 | non- | Sll0290 | leucine | ARA_LEAF | p-PcUBI | GC | 70 | 192 |
| | targeted | | | | | | | |
| 49828 | non- | Sll0290 | valine | ARA_LEAF | p-PcUBI | GC | 22 | 98 |
| | targeted | | | | | | | |
| 106920 | mitochondrial | Sll0368 | valine | ARA_LEAF | p-PcUBI | GC | 24 | 39 |
| 50104 | non- | Sll0420 | isoleucine | ARA_LEAF | p-PcUBI | GC | 41 | 69 |
| | targeted | | | | | | | |
| 50339 | plastidic | Sll0521 | valine | ARA_LEAF | p-PcUBI | GC | 21 | 56 |
| 51198 | non- | Sll0816 | isoleucine | ARA_LEAF | p-PcUBI | GC | 34 | 55 |
| | targeted | | | | | | | |
| 101707 | non- | Sll0833 | isoleucine | ARA_LEAF | p-PcUBI | GC | 39 | 124 |
| | targeted | | | | | | | |
| 85723 | mitochondrial | Sll1032 | valine | ARA_LEAF | p-PcUBI | GC | 19 | 29 |
| 53456 | non- | Sll1393 | isoleucine | ARA_LEAF | p-PcUBI | GC | 40 | 192 |
| | targeted | | | | | | | |
| 53456 | non- | Sll1393 | leucine | ARA_LEAF | p-PcUBI | GC | 99 | 132 |
| | targeted | | | | | | | |
| 53456 | non- | Sll1393 | valine | ARA_LEAF | p-PcUBI | GC | 19 | 121 |
| | targeted | | | | | | | |
| 107100 | plastidic | Sll1455 | valine | ARA_LEAF | p-PcUBI | GC | 21 | 36 |
| 95047 | plastidic | Sll1498 | isoleucine | ARA_LEAF | p-PcUBI | GC | 45 | 71 |
| 95047 | plastidic | Sll1498 | leucine | ARA_LEAF | p-PcUBI | GC | 53 | 90 |
| 55379 | non- | Sll1761 | isoleucine | ARA_LEAF | p-PcUBI | GC | 56 | 276 |
| | targeted | | | | | | | |
| 55379 | non- | Sll1761 | leucine | ARA_LEAF | p-PcUBI | GC | 67 | 245 |
| | targeted | | | | | | | |
| 85808 | mitochondrial | Sll1815 | isoleucine | ARA_LEAF | p-PcUBI | GC | 103 | 134 |
| 85808 | mitochondrial | Sll1815 | leucine | ARA_LEAF | p-PcUBI | GC | 102 | 148 |
| 85808 | mitochondrial | Sll1815 | valine | ARA_LEAF | p-PcUBI | GC | 21 | 138 |
| 86447 | plastidic | Sll1848 | isoleucine | ARA_LEAF | p-PcUBI | GC | 140 | 570 |
| 95527 | mitochondrial | Slr0150 | valine | ARA_LEAF | p-PcUBI | GC | 23 | 47 |
| 86540 | mitochondrial | Slr0239 | isoleucine | ARA_LEAF | p-PcUBI | GC | 33 | 447 |
| 86540 | mitochondrial | Slr0239 | leucine | ARA_LEAF | p-PcUBI | GC | 212 | 603 |
| 86540 | mitochondrial | Slr0239 | valine | ARA_LEAF | p-PcUBI | GC | 25 | 185 |
| 87397 | non- | Slr0721 | isoleucine | ARA_LEAF | p-PcUBI | GC | 49 | 129 |
| | targeted | | | | | | | |
| 87397 | plastidic | Slr0721 | isoleucine | ARA_LEAF | p-PcUBI | GC | 38 | 175 |
| 87397 | plastidic | Slr0721 | leucine | ARA_LEAF | p-PcUBI | GC | 98 | 267 |
| 87397 | non- | Slr0721 | leucine | ARA_LEAF | p-PcUBI | GC | 79 | 191 |
| | targeted | | | | | | | |
| 87397 | non- | Slr0721 | valine | ARA_LEAF | p-PcUBI | GC | 20 | 60 |
| | targeted | | | | | | | |
| 87397 | plastidic | Slr0721 | valine | ARA_LEAF | p-PcUBI | GC | 21 | 98 |
| 57235 | plastidic | Slr0739 | isoleucine | ARA_LEAF | p-PcUBI | GC | 37 | 120 |
| 57235 | plastidic | Slr0739 | leucine | ARA_LEAF | p-PcUBI | GC | 60 | 128 |
| 57235 | plastidic | Slr0739 | valine | ARA_LEAF | p-PcUBI | GC | 22 | 91 |
| 58668 | mitochondrial | Slr1492 | isoleucine | ARA_LEAF | p-PcUBI | GC | 27 | 42 |
| 88158 | mitochondrial | Slr1509 | isoleucine | ARA_LEAF | p-PcUBI | GC | 45 | 122 |
| 88158 | mitochondrial | Slr1509 | leucine | ARA_LEAF | p-PcUBI | GC | 59 | 144 |
| 107145 | mitochondrial | Slr1517 | valine | ARA_LEAF | p-PcUBI | GC | 27 | 33 |
| 58731 | plastidic | Slr1739 | isoleucine | ARA_LEAF | p-PcUBI | GC | 54 | 308 |
| 58731 | plastidic | Slr1739 | leucine | ARA_LEAF | p-PcUBI | GC | 148 | 331 |
| 58731 | plastidic | Slr1739 | valine | ARA_LEAF | p-PcUBI | GC | 37 | 107 |
| 12140 | plastidic | Slr1791 | valine | ARA_LEAF | p-PcUBI | GC | 25 | 33 |
| 59041 | non- | Slr1882 | valine | ARA_LEAF | p-PcUBI | GC | 20 | 27 |
| | targeted | | | | | | | |
| 12341 | mitochondrial | Slr2072 | isoleucine | ARA_LEAF | p-PcUBI | GC | 592 | 9865 |
| 12341 | plastidic | Slr2072 | isoleucine | ARA_LEAF | p-PcUBI | GC | 1626 | 10965 |
| 12341 | plastidic | Slr2072 | leucine | ARA_LEAF | p-PcUBI | GC | 50 | 250 |
| 12341 | mitochondrial | Slr2072 | leucine | ARA_LEAF | p-PcUBI | GC | 60 | 214 |
| 12341 | mitochondrial | Slr2072 | valine | ARA_LEAF | p-PcUBI | GC | 23 | 54 |
| 12341 | plastidic | Slr2072 | valine | ARA_LEAF | p-PcUBI | GC | 24 | 64 |
| 59370 | plastidic | Slr2124 | valine | ARA_LEAF | p-PcUBI | GC | 34 | 73 |
| 12698 | non- | TTC0019 | isoleucine | ARA_LEAF | p-PcUBI | GC | 43 | 847 |
| | targeted | | | | | | | |
| 12698 | non- | TTC0019 | leucine | ARA_LEAF | p-PcUBI | GC | 60 | 889 |
| | targeted | | | | | | | |
| 12698 | non- | TTC0019 | valine | ARA_LEAF | p-PcUBI | GC | 30 | 402 |
| | targeted | | | | | | | |
| 107556 | non- | TTC0849 | isoleucine | ARA_LEAF | p-PcUBI | GC | 54 | 92 |
| | targeted | | | | | | | |
| 107556 | non- | TTC0849 | leucine | ARA_LEAF | p-PcUBI | GC | 51 | 112 |
| | targeted | | | | | | | |
| 62079 | non- | TTC1386 | isoleucine | ARA_LEAF | p-PcUBI | GC | 56 | 744 |
| | targeted | | | | | | | |
| 62079 | non- | TTC1386 | leucine | ARA_LEAF | p-PcUBI | GC | 76 | 644 |
| | targeted | | | | | | | |
| 62079 | non- | TTC1386 | valine | ARA_LEAF | p-PcUBI | GC | 38 | 334 |
| | targeted | | | | | | | |
| 12974 | non- | TTC1550 | isoleucine | ARA_LEAF | p-PcUBI | GC | 70 | 355 |
| | targeted | | | | | | | |
| 12974 | non- | TTC1550 | leucine | ARA_LEAF | p-PcUBI | GC | 66 | 340 |
| | targeted | | | | | | | |
| 12974 | non- | TTC1550 | valine | ARA_LEAF | p-PcUBI | GC | 29 | 129 |
| | targeted | | | | | | | |
| 108092 | non- | Yal013w | leucine | ARA_SEED_2 | p-PcUBI | GC | 44 | 144 |
| | targeted | | | | | | | |
| 62717 | non- | Ybr160w | valine | ARA_LEAF | Big35S | GC | 20 | 55 |
| | targeted | | | | | | | |
| 108113 | plastidic | Ycr012w | isoleucine | ARA_SEED_2 | p-Super | GC | 29 | 218 |
| 108113 | plastidic | Ycr012w | leucine | ARA_SEED_2 | p-Super | GC | 33 | 136 |
| 63544 | plastidic | Ydr046c | isoleucine | ARA_LEAF | p-Super | GC | 50 | 70 |
| 63544 | plastidic | Ydr046c | valine | ARA_LEAF | p-Super | GC | 21 | 30 |
| 90103 | plastidic | Ydr183w | valine | ARA_LEAF | p-Super | GC | 30 | 48 |
| 97298 | plastidic | Yel069c | isoleucine | ARA_LEAF | p-PcUBI | GC | 38 | 58 |
| 64177 | non- | Ygl237c | isoleucine | ARA_LEAF | Big35S | GC | 39 | 294 |
| | targeted | | | | | | | |
| 64177 | non- | Ygl237c | leucine | ARA_LEAF | Big35S | GC | 80 | 355 |
| | targeted | | | | | | | |
| 108487 | non- | Ygr193c | leucine | ARA_SEED_2 | p-PcUBI | GC | 54 | 294 |
| | targeted | | | | | | | |
| 90284 | non- | Yhr006w | isoleucine | ARA_LEAF | Big35S | GC | 54 | 114 |
| | targeted | | | | | | | |
| 90284 | non- | Yhr006w | leucine | ARA_LEAF | Big35S | GC | 71 | 161 |
| | targeted | | | | | | | |
| 64470 | non- | Yhr072w-a | isoleucine | ARA_SEED_2 | p-USP | GC | 24 | 181 |
| | targeted | | | | | | | |
| 64470 | non- | Yhr072w-a | leucine | ARA_SEED_2 | p-USP | GC | 40 | 188 |
| | targeted | | | | | | | |
| 108514 | non- | Yil155c | isoleucine | ARA_SEED_2 | p-PcUBI | GC | 22 | 71 |
| | targeted | | | | | | | |
| 108514 | non- | Yil155c | leucine | ARA_SEED_2 | p-PcUBI | GC | 41 | 106 |
| | targeted | | | | | | | |
| 14885 | plastidic | Yjr139c | isoleucine | ARA_LEAF | p-PcUBI | GC | 59 | 86 |
| 90409 | plastidic | Yjr148w | isoleucine | ARA_LEAF | p-Super | GC | 109 | 273 |
| 90409 | plastidic | Yjr148w | valine | ARA_LEAF | p-Super | GC | 74 | 170 |
| 66274 | non- | Yjr153w | isoleucine | ARA_LEAF | p-PcUBI | GC | 66 | 273 |
| | targeted | | | | | | | |
| 66274 | non- | Yjr153w | leucine | ARA_LEAF | p-PcUBI | GC | 64 | 364 |
| | targeted | | | | | | | |
| 66274 | non- | Yjr153w | valine | ARA_LEAF | p-PcUBI | GC | 28 | 110 |
| | targeted | | | | | | | |
| 90602 | plastidic | YKL038W | isoleucine | ARA_LEAF | p-PcUBI | GC | 51 | 53 |
| 90602 | plastidic | YKL038W | leucine | ARA_LEAF | p-PcUBI | GC | 50 | 59 |
| 108723 | non- | Ylr224w | isoleucine | ARA_SEED_2 | p-USP | GC | 25 | 50 |
| | targeted | | | | | | | |
| 98243 | non- | Ylr348c | isoleucine | ARA_LEAF | p-PcUBI | GC | 55 | 83 |
| | targeted | | | | | | | |
| 67190 | plastidic | Ylr359w | isoleucine | ARA_LEAF | p-Super | GC | 59 | 451 |
| 67190 | plastidic | Ylr359w | leucine | ARA_LEAF | p-Super | GC | 83 | 685 |
| 67190 | plastidic | Ylr359w | valine | ARA_LEAF | p-Super | GC | 20 | 168 |
| 91785 | plastidic | Yml123c | leucine | ARA_LEAF | p-Super | GC | 65 | 96 |
| 92668 | non- | Ypl028w | isoleucine | ARA_SEED_2 | p-PcUBI | GC | 29 | 182 |
| | targeted | | | | | | | |
| 92668 | non- | Ypl028w | leucine | ARA_SEED_2 | p-PcUBI | GC | 32 | 142 |
| | targeted | | | | | | | |
| 98445 | non- | Ypr119w | isoleucine | ARA_LEAF | Big35S | GC | 33 | 57 |
| | targeted | | | | | | | |
| 68413 | non- | Zm_4842_BE510522 | isoleucine | ARA_LEAF | p-PcUBI | GC | 42 | 313 |
| | targeted | | | | | | | |
| 68413 | non- | Zm_4842_BE510522 | leucine | ARA_LEAF | p-PcUBI | GC | 53 | 336 |
| | targeted | | | | | | | |
| 68413 | non- | Zm_4842_BE510522 | valine | ARA_LEAF | p-PcUBI | GC | 29 | 116 |
| | targeted | | | | | | | |

Column 1 shows the SEQ ID NO, Column 2 shows the expression type (targeted or non-targeted), Column 3 shows the “gene name” (locus), Column 4 shows the metabolite analyzed, Column 5 indicates of the A. thaliana sourece tissue analyzed, Column 6 indicates the used promoter for expression, Column 7 indicates the analytical method. Columns 8 and 9 show the minumum and the maximum increase of the analyzed metabolite (in-percent) in comparison to the wild type (=ratio_by_weight, given as-percent increase).

When the analyses were repeated independently, all results proved to be significant.

[505.1.1.6] to [0515.1.1.6] for the disclosure of these paragraphs see [0505.1.1.1] to [0515.1.1.1] above.

In a further embodiment, the present invention relates in paragraphs [0000.1.7.7.] to [0514.1.7.7.] to a further process for the production of the fine chemical alpha-tocopherol, and/or gamma-tocopherol as defined below and corresponding embodiments as described herein as follows.

The present invention relates to a process for the production of fine chemical in a microorganism, a plant cell, a plant or a part thereof. The invention furthermore relates to nucleic acid molecules, polypeptides, nucleic acid constructs, vectors, antibodies, host cells, plant tissue, propagation material, harvested material, plants, microorganisms as well as agricultural compositions and to their use.

Due to their plastids, plants possess some biosynthetic pathways, which are, besides in cyanobacteria, unique in living organisms. Some plastidic compounds are indispensable for human and animal nutrition and are therefore called vitamins. Two essential lipophilic components for nutrition are provitamin A (beta-carotene) and vitamin E. Vitamin E is classified by its pharmacological effect and chromanol ring structure and not by biosynthesis. It comprises a class of 8 lipid-soluble components, being subdivided into tocopherols and tocotrienols. While tocopherols share an isoprenoid side chain derived from phytyl-PP, tocotrienol side chains are derivates of geranylgeranyl-PP. The α, β, γ and δ-members of these subclasses differ in their degree of methylation in the 6-chromanol-ring structure.

The tocopherol group (1a-d) has a saturated side chain, and the tocotrienol group (2a-d) has an unsaturated side chain:

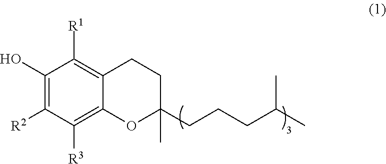

1a, α-tocopherol: R¹═R²═R³═CH₃
1b, β-tocopherol: R¹═R³═CH3, R²═H
1c, γ-tocopherol: R¹═H, R²═R³═CH₃
1d, δ-tocopherol: R¹═R²═H, R³═CH₃

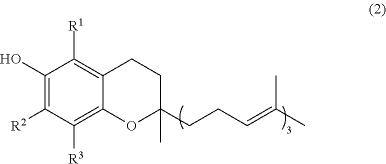

2a, α-tocotrienol: R¹═R²═R³═CH₃
2b, β-tocotrienol: R¹═R³═CH3, R²═H
2c, γ-tocotrienol: R¹═H, R²═R³═CH₃
2d, δ-tocotrienol: R¹═R²═H, R³═CH₃

In the present invention, vitamin E means all of the aforementioned tocopherols and tocotrienols with vitamin E activity or vitamin E precursor 2,3-Dimethyl-5-pythylquinol.

The four major forms of tocopherols, α, β, γ, and δ, differ in the position and number of methyl groups. The predominant form in the leaves of higher plants is α-tocopherol, whereas in seeds γ-tocopherol is often the major isoform. Tocopherols predominantly function as antioxidants in vivo in photosynthetic organisms and in animals, as well as in isolated compounds such as oils. The antioxidant properties of tocopherols derive from their ability to quench free radicals and different tocopherols may be optimal as antioxidants for different biological systems. For human and animal utility, α-tocopherol has the highest vitamin E activity and has been implicated in a variety of health areas, including possible benefits in preventing cardiovascular disease, certain cancers, and cataract formation. The amounts of vitamin E needed to achieve these effects are often quite high, 100 to 400 International Units (I.U.) and even up to 800 I.U. compared with the recommended daily allowance of 40 I.U. In fats and oils, tocopherols protect unsaturated fatty acids from oxidation. In these systems, γ-tocopherol appears to have the greater utility. In fact, tocopherols are often included in processed oils to help stabilize the fatty acids. For human health as well as food and feed utility, it is desirable to have plants with increased tocopherol content along with those where the tocopherol composition is customized.

Tocopherols contain an aromatic head group, which is derived from homogentisic acid (HGA) and a hydrocarbon portion, which arises from phytyldiphosphate (phytyl-DP). HGA is derived from the shikimic acid pathway and phytyl-DP is generated from the condensation of four isoprenoid units. The isoprenoid contribution to tocopherol biosynthesis is thought to come primarily from the plastidal methyl-erythritol phosphate pathway, and not the cytosolic mevalonic acid pathway. The condensation of HGA and phytyl-DP to form 2-methyl-6-phytylplastoquinol, the first committed step in tocopherol biosynthesis, is a prenyltransferase reaction that is performed by a homogentisate phytyltransferase (HPT). Subsequent cyclization and methylation reactions result in the formation of the four major tocopherols. The enzymatic reactions in tocopherol biosynthesis were identified 15 to 20 years ago, but cloning of the genes encoding these enzymes has only occurred in the last few years.

Tocopherol biosynthesis takes place in the plastid and the enzymes are associated with the chloroplast envelope. The membrane association of the enzymes has made purification difficult. With the advent of genomics and the availability of complete genome sequences of a number of organisms, including Synechocystis sp. PCC 6803 and Arabidopsis, it has become possible to use bioinformatics techniques to identify and clone additional genes in the tocopherol pathway.

The first enzyme cloned in the tocopherol pathway, γ-tocopherol methyl transferase (γ-TMT), was identified in Synechocystis sp. PCC 6803 and Arabidopsis using bioinformatics. In that study, the Arabidopsis γ-TMT was shown to alter seed tocopherol composition when overexpressed in Arabidopsis. γ-Tocopherol, normally the predominant tocopherol isomer in Arabidopsis seeds, was almost completely converted to α-tocopherol.

HPT catalyzes the first committed reaction in the tocopherol pathway, and was unidentified previously. Concomitant with this study, slr1736 was found to encode a HPT in Synechocystis sp. PCC 6803 and the Arabidopsis HTP was identified.

There are prenyltransferases that condense prenyl groups with allylic chains and those that condense prenyl chains with aromatic groups. The prenyltransferases that catalyze sequential condensations of isopentenylpyrophosphate with allylic chains share common features, including Asp-rich motifs, and lead to the formation of compounds with two isoprenoid units, such as geranylpyrophosphate, or to much longer molecules, such as rubber, which contains greater than 1,000 isoprenoid units. Prenyltransferases that catalyze condensations with nonisoprenoid groups have an Asp-rich motif distinct from that of the allylic class, and include UbiA, which attaches a prenyl group to 4-hydroxybenzoic acid, and chlorophyll synthase, which attaches a prenyl group to chlorophyllide.

The first committed step in tocopherol biosynthesis is catalyzed by an aromatic prenyltransferase that transfers a phytyl chain to HGA

Classification by head groups would arrange tocopherols, tocotrienols and plastoquinones in one group, being quinones with antioxidant properties and having homogentisic acid as a precursor. Plastoquinones are important components of the quinone-pool in the photosynthetic electron transport chains of plastids, also interfering in the biosynthesis of provitamin A (beta-carotene; Norris S R, (1995). Plant Cell 7, 2139-2149).

Vitamin E is predominantly delivered by the ingestion of vegetable oils. It plays an important role as a membrane-associated antioxidant scavenger. During past years several additional functions of vitamin E as anti-hypercholesterolemic and immunostimulatory agent in humans have been proposed (Beharka (1997). Methods Enzymol. 282, 247-263).

These compounds with vitamin E activity are important natural fat-soluble antioxidants. A vitamin E deficiency leads to pathophysiological situations in humans and animals. Vitamin E compounds therefore are of high economical value as additives in the food and feed sectors, in pharmaceutical formulations and in cosmetic applications.

In plastids of plants many isoprenoid pathways are localized, which are interconnected by their substrates, end products and by regulation. These are, e.g. monoterpene-, diterpene-, giberillic acid-, abscisic acid-, chlorophyll-, phylloquinone-, carotenoid-, tocopherol-, tocotrienol- and plastoquinone-biosynthesis. In all these pathways prenyltransferases are involved in the biosynthesis of these compounds. With respect to the length of their side chains diterpenes, chlorophylls, phylloquinones, tocopherols and tocotrienols can be arranged into a 020-group of isoprenoids. Another classification by degree of desaturation of the side chain, would arrange e.g. chlorophylls, phylloquinones and tocopherols into a phytyl-group and e.g. diterpenes, tocotrienols, plastoquinones and carotenoids into a group of desaturated isoprenoid compounds.

Economical methods for producing vitamin E or its precursor and food- and feedstuffs with increased vitamin E content are therefore very important.

Particularly economical methods are biotechnological methods utilizing vitamin E-producing organisms which are either natural or optimized by genetic modification.

There is a constant need for providing novel enzyme activities or direct or indirect regulators and thus alternative methods with advantageous properties for producing vitamin E or its precursor in organisms, e.g. in transgenic organisms.

Attempts are known to achieve an increase in the flow of metabolites so as to increase the tocopherol and/or tocotrienol content by overexpressing Phytyl/prenyltransferasegenes in transgenic organisms; WO 00/63391, WO 00/68393, WO 01/62781 and WO 02/33060. The content of a tocopherol and/or tocotrienol in a cell was also increased by increasing or decreasing certain activities as disclosed in WO2006/069610, WO2007/087815 or WO2008/034648 [0010.1.7.7] Improving the quality of foodstuffs and animal feeds is an important task of the food-and-feed industry. This is necessary since, for example, Vitamin E, which occur in plants are limited with regard to the supply of mammals. Especially advantageous for the quality of foodstuffs and animal feeds is as balanced as possible a vitamin profile since a great excess of some vitamins above a specific concentration in the food has no further positive effect on the utilization of the food since other vitamins suddenly become limiting. A further increase in quality is only possible via addition of further vitamins, which are limiting under these conditions. The targeted addition of the limiting vitamin in the form of synthetic products must be carried out with extreme caution in order to avoid vitamin imbalance.

To ensure a high quality of foods and animal feeds, it is therefore necessary to add a plurality of vitamins in a balanced manner to suit the respective organism. Accordingly, there is still a great demand for new and more suitable genes, which encode enzymes or regulators, which participate in the biosynthesis of vitamins, in particular vitamin E and make it possible to produce certain vitamins specifically on an industrial scale without unwanted byproducts being formed. In the selection of genes for biosynthesis or regulation two characteristics above all are particularly important. On the one hand, there is as ever a need for improved processes for obtaining the highest possible contents of vitamins like vitamin E and on the other hand as less as possible byproducts should be produced in the production process.

It is an object of the present invention to develop an inexpensive process for the synthesis of alpha-tocopherol, and/or gamma-tocopherol. Vitamin E is one of the vitamins, which are most frequently limiting.

It is a further object of the present invention to develop an inexpensive process for the synthesis of Vitamin E, preferably alpha-tocopherol, and/or gamma-tocopherol and to assure that the Vitamin E, preferably alpha-tocopherol, and/or gamma-tocopherol are more accessible and facilely to isolate and recover in an industrial scale from the producing organism, preferably from a plant.

It was now found that this object is achieved by providing the process according to the invention described herein and the embodiments characterized herein as well as in the claims.

Accordingly, in a first embodiment, the invention relates to a process for the production of at least one fine chemical selected from the group consisting of: alpha-tocopherol, and gamma-tocopherol, or, in other words, of the “fine chemical” or “fine chemical of the invention”.

The terms “fine chemical of the invention”, “fine chemical” or “the fine chemical” are used herein equally and relate in context of the paragraphs or sections [0014.1.7.7] to [0514.1.7.7] essentially to the metabolite or the metabolites indicated in column 7, application no. 7 of Tables I, II and IV in the respective line.

Further, the term “in context of any of the paragraphs [0014.1.7.7] to [0514.1.7.7]” as used herein means that for any of said paragraphs [0014.1.7.7] to [0514.1.7.7] the term “the fine chemical” is understood to follow the definition of paragraphs or sections [0014.1.7.7] and [0015.1.7.7], independently whether it refers to any other paragraph or not and whether the reference recites the term “fine chemical” in an other context.

Thus, in cases where one or more paragraphs or sections are incorporated by reference into any of the present paragraphs [0014.1.7.7] to [0514.1.7.7], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to the definition of paragraph [0014.1.7.7] and [0015.1.7.7].

Accordingly, in one embodiment, the term “fine chemical of the invention”, “fine chemical” or “the fine chemical” as used herein (alone or in combination, like in FCRP) means “alpha-tocopherol” in context of the nucleic acid or polypeptide sequences listed in the respective same line of any one of Tables Ito IV of application no. 7 and indicating in column 7 the metabolite “alpha-tocopherol”.

In one embodiment, the term alpha-tocopherol or the term “fine chemical” mean in context of the paragraphs or sections [0014.1.7.7] to [0514.1.7.7] at least one chemical compound with an activity of the above mentioned alpha-tocopherol, respectively.

Accordingly, in one embodiment, the term “fine chemical of the invention”, “fine chemical” or “the fine chemical” as used herein (alone or in combination, like in FCRP) means “gamma-tocopherol” in context of the nucleic acid or polypeptide sequences listed in the respective same line of any one of Tables Ito IV of application no. 7 and indicating in column 7 the metabolite “gamma-tocopherol”.

In one embodiment, the term gamma-tocopherol or the term “fine chemical” mean in context of the paragraphs or sections [0014.1.7.7] to [0514.1.7.7] at least one chemical compound with an activity of the above mentioned gamma-tocopherol, respectively.

Accordingly, in one embodiment, the term “fine chemical of the invention”, “fine chemical” or “the fine chemical” as used herein (alone or in combination, like in FCRP) means “Vitamin E” or its precursor 2,3-dimethyl-5-phytylquinol in context of the nucleic acid or polypeptide sequences listed in the respective same line of any one of Tables I to IV of application no. 7.

In one embodiment, the term “Vitamin E” or the term “fine chemical” mean in context of the paragraphs or sections [0014.1.7.7] to [0514.1.7.7] at least one chemical compound with an activity of the above mentioned “Vitamin E”, respectively.

Accordingly, in one embodiment, the terms “fine chemical of the invention”, “fine chemical” or “the fine chemical” mean in context of any of the paragraphs [0014.1.7.7] to [0514.1.7.7] alpha-tocopherol, its salts, ester or ether in free form or bound form. In a preferred embodiment, the term “the fine chemical” means alpha-tocopherol or its salts, ester or ether, in free form or bound form.

In a preferred embodiment “alpha-tocopherol” means alpha-tocopherol in free form.

Accordingly, in one embodiment, the terms “fine chemical of the invention”, “fine chemical” or “the fine chemical” mean in context of any of the paragraphs [0014.1.7.7] to [0514.1.7.7] gamma-tocopherol, its salts, ester or ether in free form or bound form. In a preferred embodiment, the term “the fine chemical” means gamma-tocopherol or its salts, ester or ether, in free form or bound form.

In a preferred embodiment “gamma-tocopherol” means gamma-tocopherol in free form.

In one embodiment, the term “Vitamin E” or “the fine chemical” or “the respective fine chemical” mean in context of any of the paragraphs [0014.1.7.7] to [0514.1.7.7] at least one chemical compound with vitamin E activity selected from the group “alpha-tocopherol, beta-tocopherol, gamma-tocopherol, delta-tocopherol, alpha-tocotrienol, beta-tocotrienol, gamma-tocotrienol and delta-tocotrienol” or Vitamin E precursor 2,3-dimethyl-5-phytylquinol. In an preferred embodiment, the term “the fine chemical” or the term “Vitamin E” or the term “the respective fine chemical” means at least one chemical compound with vitamin E activity selected from the group “alpha-tocopherol and gamma-tocopherol”.

Further, the term “in context of any of the paragraphs [0014.1.7.7] to [0514.1.7.7]” as used herein means that for any of said paragraphs [0014.1.7.7] to [0514.1.7.7] the term “the fine chemical” is understood to follow the definition of section [0014.1.7.7] or section [0015.1.7.7], independently whether it refers to any other paragraph or not and whether the reference recites the term “fine chemical” in an other context.

Thus, in cases where one or more paragraphs or sections are incorporated by reference into any of the present paragraphs [0014.1.7.7] to [0514.1.7.7], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to this definition of this paragraph [0015.1.7.7].

Further, the term “fine chemicals” as used herein relates to compositions comprising said fine chemical(s), i.e. comprising alpha-tocopherol, and/or gamma-tocopherol, and/or Vitamin E or its precursor 2,3-dimethyl-5-phytylquinol respectively.

An increased vitamin E content normally means an increased total vitamin E content. However, an increased vitamin E content also means, in particular, a modified content of the abovedescribed 8 compounds with vitamin E activity or its precursor 2,3-dimethyl-5-phytylquinol, without the need for an inevitable increase in the total vitamin E content. In a preferred embodiment, the term “the fine chemical” mean in context of any of the paragraphs [0014.1.7.7] to [0514.1.7.7] vitamin E in free form or its salts or its ester or ether or bound. In one embodiment, the term “the fine chemical” and the term “the respective fine chemical” mean at least one chemical compound with an activity of the above mentioned fine chemical.

Accordingly, the present invention relates to a process for the production of alpha-tocopherol, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of glucose-6-phosphate 1-dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of At1g29350-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating one or more activities selected from the group consisting of MADS box protein transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of ankyrin repeat family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating one or more activities selected from the group consisting of thioredoxin family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating one or more activities selected from the group consisting of harpin-induced family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of harpin-induced family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of monthiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of RNA-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of RNA-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of zinc finger protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of NAD-dependent epimerase/dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of ThiF family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating one or more activities selected from the group consisting of isopropylmalate isomerase large subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating one or more activities selected from the group consisting of hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of electron transfer flavoprotein subunit beta in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of threonine synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of glucose dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of glucose dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of serine protease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of geranyltranstransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of ATP-binding component of a transport system in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of 3-deoxy-D-arabinoheptulosonate-7-phosphatesynthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of major facilitator superfamily transporter protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of b1163-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of DNA polymerase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of b1219-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of b1234-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of NADP-dependent malic enzyme in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of b1522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of acid shock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating one or more activities selected from the group consisting of transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of trehalose-phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of b1921-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of O-antigen chain length determinant in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of colanic acid biosynthesis protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of b2360-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of putative transport system permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of b2548-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of b2613-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of b2673-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of murein transglycosylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of b2812-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of amino-acid acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of amino-acid acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of glycolate utilization protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating one or more activities selected from the group consisting of ABC transporter permease subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of lipopolysaccharide biosynthesis protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of b3890-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating one or more activities selected from the group consisting of glycerol dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating one or more activities selected from the group consisting of b3989-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of b3989-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of b4029-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of b4050-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating one or more activities selected from the group consisting of b4056-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of gluconate transport system permease 3 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of gluconate transport system permease 3 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of thioredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of polyphosphate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of phosphofructokinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of coproporphyrinogen oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of sll1761-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of adenylate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of precorrin methylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating one or more activities selected from the group consisting of malate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of malate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of geranylgeranyl pyrophosphate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of geranylgeranyl pyrophosphate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of carbohydrate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of photosystem II protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of glycerol-3-phosphate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of phosphoadenosine phosphosulfate reductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of slr1917-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of slr1917-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of Sec-independent protein translocase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of Sec-independent protein translocase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of multiple antibiotic resistance protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of TTC1386-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of TTC1386-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of homocitrate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating one or more activities selected from the group consisting of 3-deoxy-7-phosphoheptulonate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of 3-deoxy-7-phosphoheptulonate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of ycl049c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of homocitrate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating one or more activities selected from the group consisting of ydl235c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of N(5′-phosphoribosyl)anthranilate isomerase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of phospho-2-dehydro-3-deoxyheptonate aldolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of ydr183w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of yer106w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of small nuclear ribonucleoprotein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of phosphatidylserine decarboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating one or more activities selected from the group consisting of polygalacturonase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating one or more activities selected from the group consisting of ylr178c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating one or more activities selected from the group consisting of adenylosuccinate lyase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating one or more activities selected from the group consisting of ypr170c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

An embodiment of the present invention relates to a process for the production of alpha-tocopherol, which comprises
(a1) increasing or generating the activity of a polypeptide comprising a polypeptide as depicted in a respective line in column 5 or 8 of Table II, application no. 7, or a homolog or a fragment thereof, a consensus sequence or at least one polypeptide motif as depicted in the respective line in column 8 of Table IV, application no. 7, whereby the respective line disclose in column 7 the fine chemical alpha-tocopherol; or
(a2) increasing or generating the activity of an expression product of one or more nucleic acid molecule(s) comprising a polynucleotide as depicted in the respective line in column 5 or 8 of Table I, application no. 7, preferably the coding region thereof, or a homolog or a fragment thereof, whereby the respective line disclose in column 7 the fine chemical alpha-tocopherol; or
(a3) increasing or generating the activity of a functional equivalent of
(a1) or
(a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of alpha-tocopherol or a composition comprising alpha-tocopherol in said non-human organism or in the culture medium surrounding said non-human organism.

An embodiment of the present invention relates to a process for the production of gamma-tocopherol, which comprises
(a1) increasing or generating the activity of a polypeptide comprising a polypeptide as depicted in a respective line in column 5 or 8 of Table II, application no. 7, or a homolog or a fragment thereof, a consensus sequence or at least one polypeptide motif as depicted in the respective line in column 8 of Table IV, application no. 7, whereby the respective line disclose in column 7 the fine chemical gamma-tocopherol; or
(a2) increasing or generating the activity of an expression product of one or more nucleic acid molecule(s) comprising a polynucleotide as depicted in the respective line in column 5 or 8 of Table I, application no. 7, preferably the coding region thereof, or a homolog or a fragment thereof, whereby the respective line disclose in column 7 the fine chemical gamma-tocopherol; or
(a3) increasing or generating the activity of a functional equivalent of
(a1) or
(a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of gamma-tocopherol or a composition comprising gamma-tocopherol in said non-human organism or in the culture medium surrounding said non-human organism.

Accordingly, the increase or generation of one or more said activities is for example conferred by one or more expression products of said nucleic acid molecule, e.g. proteins. Accordingly, in the present invention described above, the increase or generation of one or more said activities is for example conferred by one or more protein(s) each comprising a polypeptide selected from the group as depicted in Table II, application no. 7, column 5 and 8, or a homolog or a fragment thereof.

The process of the invention comprises in one embodiment the following steps:
(i) increasing or generating of the expression of; and/or
(ii) increasing or generating the expression of an expression product of; and/or
(iii) increasing or generating one or more activities of an expression product encoded by;

at least one nucleic acid molecule (in the following “Fine Chemical Related Protein (FCRP)”-encoding gene or “FCRP”-gene) comprising a nucleic acid molecule selected from the group consisting of:
(a) a nucleic acid molecule encoding the polypeptide shown in column 5 or 8 of Table II, preferably Table II B, application no. 7, or a homolog or a fragment thereof;
(b) a nucleic acid molecule shown in column 5 or 8 of Table I, preferably Table I B, application no. 7, or a homolog or a fragment thereof (preferably the coding region thereof);
(c) a nucleic acid molecule, which, as a result of the degeneracy of the genetic code, can be derived from a polypeptide sequence depicted in column 5 or 8 of Table II, preferably Table II B, application no. 7;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 7, or the coding region thereof;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d),
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 7;
(i) a nucleic acid molecule encoding a polypeptide having the activity represented by a respective protein as depicted in column 5 of Table II, application no. 7;
(j) a nucleic acid molecule which comprises a polynucleotide, which is obtained by amplifying a cDNA library or a genomic library using the primers as depicted in column 8 of Table III, application no. 7; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment thereof said nucleic acid molecule encodes a polypeptide which has the activity of the polypeptide represented by a protein comprising a polypeptide as depicted in the corresponding hit in column 5 of Table II, application no. 7.

In another preferred embodiment thereof said nucleic acid molecule confers a generation or an increase of the respective fine chemical in a non-human organism or a part thereof as compared to a corresponding wild-type non-human organism or part thereof.

In another preferred embodiment thereof said nucleic acid molecule encodes a polypeptide which has the activity of the polypeptide represented by a protein as depicted in the corresponding hit in column 5 of Table II, application no. 7, and confers a generation or an increase of the respective fine chemical in a non-human organism or a part thereof as compared to a corresponding wild-type non-human organism or part thereof.

Accordingly, the genes of the present invention or used in accordance with the present invention, which respectively encode a protein having an activity of 3-deoxy-7-phosphoheptulonate synthase, 3-deoxy-D-arabinoheptulosonate-7-phosphatesynthase, ABC transporter permease subunit, acetyltransferase, acid shock protein, adenylate kinase, adenylosuccinate lyase, amino-acid acetyltransferase, ankyrin repeat family protein, At1g29350-protein, ATP-binding component of a transport system, b1163-protein, b1219-protein, b1234-protein, b1522-protein, b1921-protein, b2360-protein, b2548-protein, b2613-protein, b2673-protein, b2812-protein, b3890-protein, b3989-protein, b4029-protein, b4050-protein, b4056-protein, calcium-dependent protein kinase, carbohydrate kinase, colanic acid biosynthesis protein, coproporphyrinogen oxidase, cyclin D , DNA polymerase subunit, electron transfer flavoprotein subunit beta, geranylgeranyl pyrophosphate synthase, geranyltranstransferase, gluconate transport system permease 3, glucose dehydrogenase, glucose-6-phosphate 1-dehydrogenase, glutaredoxin, glycerol dehydrogenase, glycerol-3-phosphate dehydrogenase, glycolate utilization protein, harpin-induced family protein, homocitrate synthase, hydrolase, isopropylmalate isomerase large subunit, lipopolysaccharide biosynthesis protein, MADS box protein transcription factor, major facilitator superfamily transporter protein, malate dehydrogenase, monothiol glutaredoxin, monthiol glutaredoxin, multiple antibiotic resistance protein, murein transglycosylase, N-(5′-phosphoribosyl) anthranilate isomerase, NAD-dependent epimerase/dehydratase, NADP-dependent malic enzyme, O-antigen chain length determinant, phosphatidylserine decarboxylase, phospho-2-dehydro-3-deoxyheptonate aldolase, phosphoadenosine phosphosulfate reductase, phosphofructokinase, photosystem II protein, polygalacturonase, polyphosphate kinase, precorrin methylase, protein kinase, protein phosphatase, putative transport system permease protein, pyruvate kinase, RNA-binding protein, Sec-independent protein translocase subunit, serine protease, sll1761-protein, slr1917-protein, small nuclear ribonucleoprotein , ThiF family protein, thioredoxin, thioredoxin family protein, threonine synthase, transcription factor, transcriptional regulator, transport protein, trehalose-phosphatase, TTC1386-protein, ycl049c-protein, ydl235c-protein, ydr183w-protein, yer106w-protein, ylr178c-protein, ypr170c-protein, or zinc finger protein, which respectively encode a protein comprising a polypeptide encoded by a nucleic acid sequence as shown in Table I, application no. 7, column 5 or 8, (preferably the coding region thereof), or a homolog or a fragment thereof, which respectively encode a protein comprising a polypeptide as depicted in Table II, application no. 7, column 5 or 8, or a homolg or a fragment thereof, and/or which respectively can be amplified with the primer set shown in Table III, application no. 7, column 8, are also referred to as “FCRP genes”.

Proteins or polypeptides encoded by “FCRP-genes” are referred to as “Fine Chemical Related Proteins” or “FCRP”. For the purposes of the description of the present invention, the respective protein having an activity of 3-deoxy-7-phosphoheptulonate synthase, 3-deoxy-D-arabinoheptulosonate-7-phosphatesynthase, ABC transporter permease subunit, acetyltransferase, acid shock protein, adenylate kinase, adenylosuccinate lyase, amino-acid acetyltransferase, ankyrin repeat family protein, At1g29350-protein, ATP-binding component of a transport system, b1163-protein, b1219-protein, b1234-protein, b1522-protein, b1921-protein, b2360-protein, b2548-protein, b2613-protein, b2673-protein, b2812-protein, b3890-protein, b3989-protein, b4029-protein, b4050-protein, b4056-protein, calcium-dependent protein kinase, carbohydrate kinase, colanic acid biosynthesis protein, coproporphyrinogen oxidase, cyclin D , DNA polymerase subunit, electron transfer flavoprotein subunit beta, geranylgeranyl pyrophosphate synthase, geranyltranstransferase, gluconate transport system permease 3, glucose dehydrogenase, glucose-6-phosphate 1-dehydrogenase, glutaredoxin, glycerol dehydrogenase, glycerol-3-phosphate dehydrogenase, glycolate utilization protein, harpin-induced family protein, homocitrate synthase, hydrolase, isopropylmalate isomerase large subunit, lipopolysaccharide biosynthesis protein, MADS box protein transcription factor, major facilitator superfamily transporter protein, malate dehydrogenase, monothiol glutaredoxin, monthiol glutaredoxin, multiple antibiotic resistance protein, murein transglycosylase, N-(5′-phosphoribosyl)anthranilate isomerase, NAD-dependent epimerase/dehydratase, NADP-dependent malic enzyme, O-antigen chain length determinant, phosphatidylserine decarboxylase, phospho-2-dehydro-3-deoxyheptonate aldolase, phosphoadenosine phosphosulfate reductase , phosphofructokinase, photosystem II protein, polygalacturonase, polyphosphate kinase, precorrin methylase, protein kinase, protein phosphatase, putative transport system permease protein, pyruvate kinase, RNA-binding protein, Sec-independent protein translocase subunit, serine protease, sll1761-protein, slr1917-protein, small nuclear ribonucleoprotein , ThiF family protein, thioredoxin, thioredoxin family protein, threonine synthase, transcription factor, transcriptional regulator, transport protein, trehalose-phosphatase, TTC1386-protein, ycl049c-protein, ydl235c-protein, ydr183w-protein, yer106w-protein, ylr178c-protein, ypr170c-protein, or zinc finger protein, the respective protein comprising a polypeptide encoded by one or more respective nucleic acid sequences as shown in Table I, application no. 7, column 5 or 8, (preferably the coding region thereof), or a homolog or fragment thereof, the respective protein comprising a respective polypeptide as depicted in Table II, application no. 7, column 5 or 8, or a homolog or fragment thereof, the respective protein comprising a sequence corresponding to the consensus sequence as shown in Table IV, application no. 7, column 8, and/or the respective protein comprising at least one polypeptide motif as shown in Table IV, application no. 7, column 8 is also referred to as Fine Chemical Related Protein” or “FCRP”.

Thus, in one embodiment, the present invention provides a process of the production of alpha-tocopherol, and/or gamma-tocopherol, by increasing or generating one or more activities, especially selected from the group consisting of 3-deoxy-7-phosphoheptulonate synthase, 3-deoxy-D-arabinoheptulosonate-7-phosphatesynthase, ABC transporter permease subunit, acetyltransferase, acid shock protein, adenylate kinase, adenylosuccinate lyase, amino-acid acetyltransferase, ankyrin repeat family protein, At1g29350-protein, ATP-binding component of a transport system, b1163-protein, b1219-protein, b1234-protein, b1522-protein, b1921-protein, b2360-protein, b2548-protein, b2613-protein, b2673-protein, b2812-protein, b3890-protein, b3989-protein, b4029-protein, b4050-protein, b4056-protein, calcium-dependent protein kinase, carbohydrate kinase, colanic acid biosynthesis protein, coproporphyrinogen oxidase, cyclin D , DNA polymerase subunit, electron transfer flavoprotein subunit beta, geranylgeranyl pyrophosphate synthase, geranyltranstransferase, gluconate transport system permease 3, glucose dehydrogenase, glucose-6-phosphate 1-dehydrogenase, glutaredoxin, glycerol dehydrogenase, glycerol-3-phosphate dehydrogenase, glycolate utilization protein, harpininduced family protein, homocitrate synthase, hydrolase, isopropylmalate isomerase large subunit, lipopolysaccharide biosynthesis protein, MADS box protein transcription factor, major facilitator superfamily transporter protein, malate dehydrogenase, monothiol glutaredoxin, monthiol glutaredoxin, multiple antibiotic resistance protein, murein transglycosylase, N-(5′-phosphoribosyl) anthranilate isomerase, NAD-dependent epimerase/dehydratase, NADP-dependent malic enzyme, O-antigen chain length determinant, phosphatidylserine decarboxylase, phospho-2-dehydro-3-deoxyheptonate aldolase, phosphoadenosine phosphosulfate reductase , phosphofructokinase, photosystem II protein, polygalacturonase, polyphosphate kinase, precorrin methylase, protein kinase, protein phosphatase, putative transport system permease protein, pyruvate kinase, RNA-binding protein, Sec-independent protein translocase subunit, serine protease, sll1761-protein, slr1917-protein, small nuclear ribonucleoprotein , ThiF family protein, thioredoxin, thioredoxin family protein, threonine synthase, transcription factor, transcriptional regulator, transport protein, trehalose-phosphatase, TTC1386-protein, ycl049c-protein, ydl235c-protein, ydr183w-protein, yer106w-protein, ylr178c-protein, ypr170c-protein, and zinc finger protein, which is conferred by one or more FCRPs or the gene product of one or more FCRP-genes, for example by the gene product of a nucleic acid sequences comprising a polynucleotide selected from the group as shown in Table I, application no. 7, column 5 or 8, (preferably by the coding region thereof), or a homolog or a fragment thereof, e.g. or by one or more proteins each comprising a polypeptide encoded by one or more nucleic acid sequences selected from the group as shown in Table I, application no. 7, column 5 or 8, (preferably by the coding region thereof), or a homolog or a fragment thereof, or by one or more protein(s) each comprising a polypeptide selected from the group as depicted in Table II, application no. 7, column 5 and 8, or a homolog thereof, or a protein comprising a sequence corresponding to the consensus sequence or comprising at least one polypeptide motif as shown in Table IV, application no. 7, column 8.

As mentioned, the process for the production of the fine chemical according to the present invention, in particular showing a generation or an increase of the respective fine chemical in a non-human organism or a part thereof as compared to a corresponding wild-type non-human organism or part thereof, can be mediated by one or more FCRP-genes or FCPRs.

In an embodiment, the process comprises increasing or generating the activity of one or more polypeptides having said activity, e.g.

by generating or increasing the amount and/or specific activity in the cell or a compartment of a cell of one of more FCRP, especially selected from the group consisting of 3-deoxy-7-phosphoheptulonate synthase, 3-deoxy-D-arabinoheptulosonate-7-phosphatesynthase, ABC transporter permease subunit, acetyltransferase, acid shock protein, adenylate kinase, adenylosuccinate lyase, amino-acid acetyltransferase, ankyrin repeat family protein, At1g29350-protein,

ATP-binding component of a transport system, b1163-protein, b1219-protein, b1234-protein, b1522-protein, b1921-protein, b2360-protein, b2548-protein, b2613-protein, b2673-protein, b2812-protein, b3890-protein, b3989-protein, b4029-protein, b4050-protein, b4056-protein, calcium-dependent protein kinase, carbohydrate kinase, colanic acid biosynthesis protein, coproporphyrinogen oxidase, cyclin D , DNA polymerase subunit, electron transfer flavoprotein subunit beta, geranylgeranyl pyrophosphate synthase, geranyltranstransferase, gluconate transport system permease 3, glucose dehydrogenase, glucose-6-phosphate 1-dehydrogenase, glutaredoxin, glycerol dehydrogenase, glycerol-3-phosphate dehydrogenase, glycolate utilization protein, harpin-induced family protein, homocitrate synthase, hydrolase, isopropylmalate isomerase large subunit, lipopolysaccharide biosynthesis protein, MADS box protein transcription factor, major facilitator superfamily transporter protein, malate dehydrogenase, monothiol glutaredoxin, monthiol glutaredoxin, multiple antibiotic resistance protein, murein transglycosylase, N-(5′-phosphoribosyl) anthranilate isomerase, NAD-dependent epimerase/dehydratase, NADP-dependent malic enzyme, O-antigen chain length determinant, phosphatidylserine decarboxylase, phospho-2-dehydro-3-deoxyheptonate aldolase, phosphoadenosine phosphosulfate reductase , phosphofructokinase, photosystem II protein, polygalacturonase, polyphosphate kinase, precorrin methylase, protein kinase, protein phosphatase, putative transport system permease protein, pyruvate kinase, RNA-binding protein, Sec-independent protein translocase subunit, serine protease, sll1761-protein, slr1917-protein, small nuclear ribonucleoprotein , ThiF family protein, thioredoxin, thioredoxin family protein, threonine synthase, transcription factor, transcriptional regulator, transport protein, trehalose-phosphatase, TTC1386-protein, ycl049c-protein, ydl235c-protein, ydr183w-protein, yer106w-protein, ylr178c-protein, ypr170c-protein, and zinc finger protein, for example of the respective polypeptide as depicted in Table II, application no. 7, column 5 and 8, or a homolog or a fragment thereof, or the respective polypeptide comprising a s equence corresponding to the consensus sequences as shown in Table IV, application no. 7, column 8, or the respective polypeptide comprising at least one polypeptide motif as depicted in Table IV, application no. 7, column 8.

A further embodiment of the present invention relates to a process for the production of alpha-tocopherol, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a glucose-6-phosphate 1-dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a At1g29350-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating the activity of a MADS box protein transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a ankyrin repeat family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating the activity of a thioredoxin family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating the activity of a harpin-induced family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a harpin-induced family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a monthiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating the activity of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a RNA-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a RNA-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a zinc finger protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a NAD-dependent epimerase/dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a ThiF family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating the activity of a isopropylmalate isomerase large subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating the activity of a hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a electron transfer flavoprotein subunit beta non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a threonine synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a glucose dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a glucose dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a serine protease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a geranyltranstransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a ATP-binding component of a transport system non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a 3-deoxy-D-arabinoheptulosonate-7-phosphatesynthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a major facilitator superfamily transporter protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a b1163-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a DNA polymerase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a b1219-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a b1234-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a NADP-dependent malic enzyme non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a b1522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a acid shock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating the activity of a transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a trehalose-phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a b1921-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a O-antigen chain length determinant non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a colanic acid biosynthesis protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a b2360-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a putative transport system permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a b2548-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a b2613-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a b2673-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a murein transglycosylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a b2812-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a amino-acid acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a amino-acid acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a glycolate utilization protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating the activity of a ABC transporter permease subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a lipopolysaccharide biosynthesis protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a b3890-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating the activity of a glycerol dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating the activity of a b3989-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a b3989-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a b4029-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a b4050-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating the activity of a b4056-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a gluconate transport system permease 3 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a gluconate transport system permease 3 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a thioredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a polyphosphate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a phosphofructokinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a coproporphyrinogen oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a sll1761-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a adenylate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a precorrin methylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating the activity of a malate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a malate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a geranylgeranyl pyrophosphate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a geranylgeranyl pyrophosphate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a carbohydrate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a photosystem II protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a glycerol-3-phosphate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a phosphoadenosine phosphosulfate reductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a slr1917-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a slr1917-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a Sec-independent protein translocase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a Sec-independent protein translocase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a multiple antibiotic resistance protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a TTC1386-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a TTC1386-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a homocitrate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating the activity of a 3-deoxy-7-phosphoheptulonate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a 3-deoxy-7-phosphoheptulonate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a ycl049c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a homocitrate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating the activity of a ydl235c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a N-(5′-phosphoribosyl)anthranilate isomerase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a phospho-2-dehydro-3-deoxyheptonate aldolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a ydr183w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a yer106w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a small nuclear ribonucleoprotein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a phosphatidylserine decarboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a polygalacturonase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating the activity of a ylr178c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating the activity of a adenylosuccinate lyase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating the activity of a ypr170c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating the activity of a polypeptide comprising a polypeptide as depicted in the respective line in column 5 or 8 of Table II, application no. 7, or a homolog or a fragment thereof, a consensus sequence or at least one polypeptide motif as depicted in the respective line in column 8 of Table IV, application no. 7, whereby the respective line disclose in column 7 the fine chemical alpha-tocopherol; or
increasing or generating the activity of an expression product of one or more nucleic acid molecule(s) comprising a polynucleotide as depicted in the respective line in column 5 or 8 of Table I, application no. 7, preferably the coding region thereof, or a homolog or a fragment thereof, whereby the respective line disclose in column 7 the fine chemical alpha-tocopherol;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of alpha-tocopherol, or a composition comprising alpha-tocopherol in said non-human organism or in the culture medium surrounding said non-human organism.

A further embodiment of the present invention relates to a process for the production of gamma-tocopherol, which comprises
(a) increasing or generating the activity of a polypeptide comprising a polypeptide as depicted in the respective line in column 5 or 8 of Table II, application no. 7, or a homolog or a fragment thereof, a consensus sequence or at least one polypeptide motif as depicted in the respective line in column 8 of Table IV, application no. 7, whereby the respective line disclose in column 7 the fine chemical gamma-tocopherol; or
increasing or generating the activity of an expression product of one or more nucleic acid molecule(s) comprising a polynucleotide as depicted in the respective line in column 5 or 8 of Table I, application no. 7, preferably the coding region thereof, or a homolog or a fragment thereof, whereby the respective line disclose in column 7 the fine chemical gamma-tocopherol;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of gamma-tocopherol, or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of glucose-6-phosphate 1-dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of At1g29350-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of MADS box protein transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of ankyrin repeat family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of thioredoxin family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of harpin-induced family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of harpin-induced family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of monthiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of RNA-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of RNA-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of zinc finger protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of NAD-dependent epimerase/dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of ThiF family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of isopropylmalate isomerase large subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of electron transfer flavoprotein subunit beta in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of threonine synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of glucose dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of glucose dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of serine protease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of geranyltranstransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of ATP-binding component of a transport system in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of 3-deoxy-D-arabinoheptulosonate-7-phosphatesynthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of major facilitator superfamily transporter protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of b1163-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of DNA polymerase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of b1219-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of b1234-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of NADP-dependent malic enzyme in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of b1522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of acid shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of trehalose-phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of b1921-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of O-antigen chain length determinant in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of colanic acid biosynthesis protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of b2360-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of putative transport system permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of b2548-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of b2613-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of b2673-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of murein transglycosylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of b2812-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of amino-acid acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of amino-acid acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of glycolate utilization protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of ABC transporter permease subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of lipopolysaccharide biosynthesis protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of b3890-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of glycerol dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of b3989-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of b3989-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of b4029-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of b4050-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of b4056-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of gluconate transport system permease 3 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of gluconate transport system permease 3 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of thioredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of polyphosphate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of phosphofructokinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of coproporphyrinogen oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of sll1761-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of adenylate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of precorrin methylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of malate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of malate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of geranylgeranyl pyrophosphate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of geranylgeranyl pyrophosphate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of carbohydrate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of photosystem II protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of glycerol-3-phosphate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of phosphoadenosine phosphosulfate reductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of slr1917-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of slr1917-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of Sec-independent protein translocase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of Sec-independent protein translocase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of multiple antibiotic resistance protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of TTC1386-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of TTC1386-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of homocitrate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of 3-deoxy-7-phosphoheptulonate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of 3-deoxy-7-phosphoheptulonate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of ycl049c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of homocitrate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of ydl235c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of N(5′-phosphoribosyl)anthranilate isomerase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of phospho-2-dehydro-3-deoxyheptonate aldolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of ydr183w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of yer106w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of small nuclear ribonucleoprotein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of phosphatidylserine decarboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of polygalacturonase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of gamma-tocopherol, which comprises
(a) increasing or generating one or more activities selected from the group consisting of ylr178c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of adenylosuccinate lyase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a) increasing or generating one or more activities selected from the group consisting of ypr170c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

A further embodiment of the present invention relates to a process for the production of alpha-tocopherol, which comprises
(a1) increasing or generating the activity of a polypeptide comprising a polypeptide as depicted in the respective line in column 5 or 8 of Table II, application no. 7, or a homolog or fragment thereof, a consensus sequence or at least one polypeptide motif as depicted in column 8 of Table IV, application no. 7, whereby the respective line disclose in column 7 the fine chemical alpha-tocopherol; or
increasing or generating the activity of an expression product of one or more nucleic acid molecule(s) comprising a polynucleotide as depicted in the respective line in column 5 or 8 of Table I, application no. 7, preferably the coding region thereof, or a homolog or a fragment thereof, whereby the respective line disclose in column 7 the fine chemical alpha-tocopherol;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 7, or a homolog or a fragment thereof, a consensus sequence or at least one polypeptide motif as depicted in the respective line in column 8 of Table IV, application no. 7, which is joined to a transit peptide, whereby the respective line disclose in column 7 the fine chemical alpha-tocopherol; or
increasing or generating the activity of an expression product of one or more nucleic acid molecule(s) comprising a polynucleotide as depicted in the respective line in column 5 or 8 of Table I, application no. 7, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical alpha-tocopherol;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 7, or a homolog or a fragment thereof, a consensus sequence or at least one polypeptide motif as depicted in the respective line in column 8 of Table IV, application no. 7, whereby the respective line disclose in column 7 the fine chemical alpha-tocopherol; or
increasing or generating the activity of an expression product of one or more nucleic acid molecule(s) comprising a polynucleotide as depicted in the respective line in column 5 or 8 of Table I, application no. 7, preferably the coding region thereof, or a homolog or a fragment thereof, whereby the respective line disclose in column 7 the fine chemical alpha-tocopherol;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of alpha-tocopherol, or a composition comprising alpha-tocopherol in said non-human organism or in the culture medium surrounding said non-human organism.

A further embodiment of the present invention relates to a process for the production of gamma-tocopherol, which comprises
(a1) increasing or generating the activity of a polypeptide comprising a polypeptide as depicted in the respective line in column 5 or 8 of Table II, application no. 7, or a homolog or fragment thereof, a consensus sequence or at least one polypeptide motif as depicted in column 8 of Table IV, application no. 7, whereby the respective line disclose in column 7 the fine chemical gamma-tocopherol; or
increasing or generating the activity of an expression product of one or more nucleic acid molecule(s) comprising a polynucleotide as depicted in the respective line in column 5 or 8 of Table I, application no. 7, preferably the coding region thereof, or a homolog or a fragment thereof, whereby the respective line disclose in column 7 the fine chemical gamma-tocopherol;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 7, or a homolog or a fragment thereof, a consensus sequence or at least one polypeptide motif as depicted in the respective line in column 8 of Table IV, application no. 7, which is joined to a transit peptide, whereby the respective line disclose in column 7 the fine chemical gamma-tocopherol; or
increasing or generating the activity of an expression product of one or more nucleic acid molecule(s) comprising a polynucleotide as depicted in the respective line in column 5 or 8 of Table I, application no. 7, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical gamma-tocopherol;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 7, or a homolog or a fragment thereof, a consensus sequence or at least one polypeptide motif as depicted in the respective line in column 8 of Table IV, application no. 7, whereby the respective line disclose in column 7 the fine chemical gamma-tocopherol; or
increasing or generating the activity of an expression product of one or more nucleic acid molecule(s) comprising a polynucleotide as depicted in the respective line in column 5 or 8 of Table I, application no. 7, preferably the coding region thereof, or a homolog or a fragment thereof, whereby the respective line disclose in column 7 the fine chemical gamma-tocopherol;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of gamma-tocopherol, or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a glucose-6-phosphate 1-dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a At1g29350-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating the activity of a MADS box protein transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a ankyrin repeat family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating the activity of a thioredoxin family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating the activity of a harpin-induced family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a harpin-induced family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a monthiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a RNA-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a RNA-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a zinc finger protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a NAD-dependent epimerase/dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a ThiF family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating the activity of a isopropylmalate isomerase large subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating the activity of a hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a electron transfer flavoprotein subunit beta in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a threonine synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a glucose dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a glucose dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a serine protease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a geranyltranstransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a ATP-binding component of a transport system in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a 3-deoxy-D-arabinoheptulosonate-7-phosphatesynthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a major facilitator superfamily transporter protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a b1163-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a DNA polymerase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a b1219-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a b1234-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a NADP-dependent malic enzyme in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a b1522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a acid shock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating the activity of a transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a trehalose-phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a b1921-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a O-antigen chain length determinant in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a colanic acid biosynthesis protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a b2360-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a putative transport system permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a b2548-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a b2613-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a b2673-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a murein transglycosylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a b2812-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a amino-acid acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a amino-acid acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a glycolate utilization protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating the activity of a ABC transporter permease subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a lipopolysaccharide biosynthesis protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a b3890-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating the activity of a glycerol dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating the activity of a b3989-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a b3989-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a b4029-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a b4050-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating the activity of a b4056-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a gluconate transport system permease 3 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a gluconate transport system permease 3 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a thioredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a polyphosphate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a phosphofructokinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a coproporphyrinogen oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a sll1761-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a adenylate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a precorrin methylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating the activity of a malate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a malate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a geranylgeranyl pyrophosphate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a geranylgeranyl pyrophosphate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a carbohydrate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a photosystem II protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a glycerol-3-phosphate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a phosphoadenosine phosphosulfate reductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a slr1917-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a slr1917-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a Sec-independent protein translocase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a Sec-independent protein translocase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a multiple antibiotic resistance protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a TTC1386-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a TTC1386-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a homocitrate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating the activity of a 3-deoxy-7-phosphoheptulonate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a 3-deoxy-7-phosphoheptulonate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a ycl049c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a homocitrate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating the activity of a ydl235c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a N-(5′-phosphoribosyl)anthranilate isomerase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a phospho-2-dehydro-3-deoxyheptonate aldolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a ydr183w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a yer106w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a small nuclear ribonucleoprotein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a phosphatidylserine decarboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a polygalacturonase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of gamma-tocopherol, which comprises
(a) increasing or generating the activity of a ylr178c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of gamma-tocopherol or a composition comprising gamma-tocopherol in said non-human organism or in the culture medium surrounding said non-human organism.

Accordingly, the present invention relates to a process for the production of alpha-tocopherol, which comprises
(a) increasing or generating the activity of a adenylosuccinate lyase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating the activity of a ypr170c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol or a composition comprising alpha-tocopherol in said non-human organism or in the culture medium surrounding said non-human organism.

Accordingly, the present invention relates to a process for the production of alpha-tocopherol, which comprises
(a) increasing or generating the activity of a polypeptide comprising a polypeptide as depicted in the respective line in column 5 or 8 of Table II, application no. 7, or a homolog or a fragment thereof, a consensus sequence or at least one polypeptide motif as depicted in the respective line in column 8 of Table IV, application no. 7, whereby the respective line disclose in column 7 the fine chemical alpha-tocopherol; or
increasing or generating the activity of an expression product of one or more nucleic acid molecule(s) comprising a polynucleotide as depicted in the respective line in column 5 or 8 of Table I, application no. 7, preferably the coding region thereof, or a homolog or a fragment thereof, whereby the respective line disclose in column 7 the fine chemical alpha-tocopherol;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of alpha-tocopherol, or a composition comprising alpha-tocopherol in said non-human organism or in the culture medium surrounding said non-human organism.

Accordingly, the present invention relates to a process for the production of gamma-tocopherol, which comprises
(a) increasing or generating the activity of a polypeptide comprising a polypeptide as depicted in the respective line in column 5 or 8 of Table II, application no. 7, or a homolog or a fragment thereof, a consensus sequence or at least one polypeptide motif as depicted in the respective line in column 8 of Table IV, application no. 7, whereby the respective line disclose in column 7 the fine chemical gamma-tocopherol; or
increasing or generating the activity of an expression product of one or more nucleic acid molecule(s) comprising a polynucleotide as depicted in the respective line in column 5 or 8 of Table I, application no. 7, preferably the coding region thereof, or a homolog or a fragment thereof, whereby the respective line disclose in column 7 the fine chemical gamma-tocopherol;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of gamma-tocopherol, or a composition comprising gamma-tocopherol in said non-human organism or in the culture medium surrounding said non-human organism.

In a further embodiment the activity of the polypeptide comprising a polypeptide as depicted in the respective line in column 5 or 8 of Table II, application no. 7, or a homolog or a fragment thereof, a consensus sequence or at least one polypeptide motif as depicted in the respective line in column 8 of Table IV, application no. 7, is increased or generated non-targeted in the above-mentioned process in a microorganism or plant or a part thereof.

In a further embodiment said polypeptide has the activity of the respective polypeptide represented by a protein comprising a polypeptide as depicted in the respective line in column 5 of Table II, application no. 7.

In a further embodiment the activity of the expression product of one or more nucleic acid molecule(s) comprising a polynucleotide as depicted in the respective line in column 5 or 8 of Table I, application no. 7, preferably the coding region thereof, or a homolog or a fragment thereof, is increased or generated non-targeted in the above-mentioned process in a microorganism or plant or a part thereof.

In a further embodiment the activity of the polypeptide comprising a polypeptide as depicted in the respective line in column 5 or 8 of Table II, application no. 7, or a homolog or a fragment thereof, a consensus sequence or at least one polypeptide motif as depicted in the respective line in column 8 of Table IV, application no. 7, is increased or generated in the above-mentioned process in an organelle preferably in plastids or mitochondria, especially in plastids, of a microorganism or plant.

In a further embodiment said polypeptide has the activity of the respective polypeptide represented by a protein comprising a polypeptide as depicted in the respective line in column 5 of Table II, application no. 7.

In a further embodiment the activity of the expression product of one or more nucleic acid molecule(s) comprising a polynucleotide as depicted in the respective line in column 5 or 8 of Table I, application no. 7, preferably the coding region thereof, or a homolog or a fragment thereof, is increased or generated in the above-mentioned process in an organelle, preferably in plasids or mitochondria, especially in plastids, of a microorganism or plant.

In a further embodiment the activity of the polypeptide comprising a polypeptide as depicted in the respective line in column 5 or 8 of Table II, application no. 7, or a homolog or a fragment thereof, a consensus sequence or at least one polypeptide motif as depicted in the respective line in column 8 of Table IV, application no. 7, is increased or generated in the above-mentioned process in the cyctosol of a cell, of a microorganism or plant.

In a further embodiment said polypeptide has the activity of the respective polypeptide represented by a protein comprising a polypeptide as depicted in the respective line in column 5 of Table II, application no. 7.

In a further embodiment the activity of the expression product of one or more nucleic acid molecule(s) comprising a polynucleotide as depicted in the respective line in column 5 or 8 of Table I, application no. 7, preferably the coding region thereof, or a homolog or a fragment thereof, is increased or generated in the above-mentioned process in the cytosol of a cell, of a microorganism or plant.

In a further embodiment of the present invention the process further comprises the step of recovering the fine chemical, which is synthesized by the organism from the organism and/or from the culture medium used for the growth or maintenance of the organism.

For the purposes of the present invention, as a rule the plural is intended to encompass the singular and vice versa, unless otherwise specified.

For the purposes of the present invention, as a rule the term “mitochondric” is intended to encompass the term “mitochondrial” and vice versa, unless otherwise specified.

Comprises/comprising and grammatical variations thereof when used in this specification are to be taken to specify the presence of stated features, integers, steps or components or groups thereof, but not to preclude the presence or addition of one or more other features, integers, steps, components or groups thereof.

The term “Table I” used in this specification is to be taken to specify the content of Table I A and Table I B. The term “Table II” used in this specification is to be taken to specify the content of Table II A and Table II B. The term “Table I A” used in this specification is to be taken to specify the content of Table I A. The term “Table I B” used in this specification is to be taken to specify the content of Table I B. The term “Table II A” used in this specification is to be taken to specify the content of Table II A. The term “Table II B” used in this specification is to be taken to specify the content of Table II B. In one preferred embodiment, the term “Table I” means Table I B. In one preferred embodiment, the term “Table II” means Table II B.

In a line of Table I related nucleic acid molecules are listed. In column 3 the locus name, often also referred to as gene name, is given, in column 5 the lead sequence ID No. thereto and in column 8 the sequence ID No. of homologues thereof. In the corresponding line of Table II the respective polypeptides are listed. In column 3 the protein name is given (which is according to the common understanding of the skilled person in the art usually used for the gene as well as the polypeptide and therefore identical with the gene name/locus name), in column 5 the (corresponding) lead sequence ID No. thereto and in column 8 the (corresponding) sequence ID No. of homologues thereof.

In Tables I and II in column 4 information is given from which organism the lead sequence according to column 5 has been identified, in column 7 information is given which fine chemical is generated or increased, and in an especial embodiment in column 6 information is given about non-targeted expression or expression in plastids or mitochondria.

Tables III and IV are arranged accordingly whereby in column 8 of table III primers are listed which can be used to amplify the sequence of the corresponding lead sequence indicated in column 5 of the same line and whereby in column 8 of table IV consensus and pattern sequences are listed which are shared by the lead sequence as indicated in column 5 of the same line and their homologs listed in the same line in table II column 8. How theconsensus and pattern sequences are determined to be shared by their respective lead or homolog sequence is defined in a respective section of the application in more detail.

The terms “increase”, “raise”, “extend”, “enhance”, “improve” and “amplify” as well as the grammatical versions thereof relate to a corresponding change of a property in a non-human organism, a part of an organism such as a tissue, seed, root, leave, flower, pollen etc. or in a cell and are interchangeable. Preferably, the overall activity in the volume is increased or enhanced in cases if the increase or enhancement is related to the increase or enhancement of an activity of a gene product, independent whether the amount of gene product or the specific activity of the gene product or both is increased or enhanced or whether the amount, stability or translation efficacy of the nucleic acid sequence or gene encoding for the gene product is increased or enhanced.

Under “change of a property” it is understood that the activity, expression level or amount of a gene product or the metabolite content is changed in a specific volume relative to a corresponding volume of a control, reference or wild type, including the de novo creation of the activity or expression.

The term “increase” may be directed to a change of said property in the subject of the present invention or only in a part thereof, for example, the change can be found in a compartment of a cell, like an organelle, or in a part of an non-human organism, like plant tissue, plant seed, plant root, pollen, leave, flower etc. but is not detectable in the overall subject, i.e. complete cell or plant, if tested.

Accordingly, the term “increase” means that the specific activity of a polypeptide or the amount of a compound or of a metabolite, e.g. of a polypeptide, a nucleic acid molecule or an encoding mRNA or DNA or the fine chemical, can be increased in a volume.

The term “increase” includes that a compound or an activity is introduced into a cell or a subcellular compartment or organelle de novo or that the compound or the activity has not been detectable before, in other words it is “generated”.

Accordingly, in the following, the term “increasing” also comprises the term “generating” or “stimulating”. The increased activity manifests itself in an increase of the fine chemical.

The terms “wild type”, “control” or “reference” are exchangeable and can be a cell or a part of a non-human organism such as an organelle like a chloroplast or a tissue, or a non-human organism, in particular a microorganism or a plant, which was not modified or treated according to the herein described process according to the invention. Accordingly, the cell or a part of a non-human organism such as an organelle like a chloroplast or a tissue, or an organism, in particular a microorganism or a plant used as wild type, control or reference corresponds to the cell, non-human organism, microorganism, plant, or a part thereof, as much as possible and is in any other property but in the result of the process of the invention as identical to the subject matter of the invention as possible. Thus, the wild type, control or reference is treated identically or as identical as possible, saying that only conditions or properties might be different which do not influence the quality of the tested property.

Preferably, any comparison is carried out under analogous conditions. The term “analogous conditions” means that all conditions such as, for example, culture or growing conditions (such as soil, nutrient, water content of the soil, temperature, humidity or surrounding air), or assay conditions (such as buffer composition, temperature, substrates, pathogen strain, concentrations and the like) are kept identical between the experiments to be compared.

The “reference”, “control” or “wild type” is preferably a subject, e.g. an organelle, a cell, a tissue, an organism, in particular a microorganism or a plant, which was not modified or treated according to the herein described process of the invention and is in any other property as similar to the subject matter of the invention as possible. The reference, control or wild type is in its genome, transcriptome, proteome or metabolome as similar as possible to the subject of the present invention. Preferably, the term “reference-”, “control-” or “wild type-”organelle, -cell, -tissue or -organism, in particular a microorganism or a plant, relates to an organelle, cell, tissue or organism, in particular a microorganism or a plant, which is nearly genetically identical to the organelle, cell, tissue or organism, in particular a microorganism or plant, or a part thereof, of the present invention, preferably 95%, more preferred are 98%, even more preferred are 99,00%, in particular 99,10%, 99,30%, 99,50%, 99,70%, 99.90%, 99.99%, 99.999% or more. Most preferable the “reference”, “control”, or “wild type” is a subject, e.g. an organelle, a cell, a tissue, an organism, in particular a microorganism or a plant, which is genetically identical to the organism, in particular plant, cell, a tissue or organelle used according to the process of the invention except that the responsible or activity conferring nucleic acid molecules or the gene product encoded by them are amended, manipulated, exchanged or introduced according to the inventive process.

In case, a control, reference or wild type differing from the subject of the present invention only by not being subject of the process of the invention can not be provided, a control, reference or wild type can be a non-human organism in which the cause for the modulation of an activity conferring the generation/increase of the fine chemical or expression of the nucleic acid molecule of the invention as described herein has been switched back or off, e.g. by knocking out the expression of responsible gene product, e.g. by antisense inhibition, by inactivation of an activator or agonist, by activation of an inhibitor or antagonist, by inhibition through adding inhibitory antibodies, by adding active compounds as e.g. hormones, by introducing negative dominant mutants, etc. A gene production can for example be knocked out by introducing inactivating point mutations, which lead to an enzymatic activity inhibition or a destabilization or an inhibition of the ability to bind to cofactors etc.

Accordingly, preferred reference subject is the starting subject of the present process of the invention. Preferably, the reference and the subject matter of the invention are compared after standardization and normalization, e.g. to the amount of total RNA, DNA, or protein or activity or expression of reference genes, like housekeeping genes, such as ubiquitin, actin or ribosomal proteins.

In accordance with the invention, the term “non-human organism” as understood herein relates always to an organism with the exception of a human being, in particular to an animal or a plant or a microorganism. Further, the term “animal” as understood herein relates always to a non-human animal. Preferably the term “non-human organism” shall mean a microorganism, in particular those containing plastids, such as algae, or a plant or the like.

As used herein, “plant” is meant to include not only a whole plant but also a part thereof i.e., one or more cells, tissues, including for example, leaves, stems, shoots, roots, flowers, fruits, seeds and pollen.

The term “organelle” according to the invention shall mean for example “mitochondrion” or “plastid”. The term “plastid” according to the invention is intended to include various forms of plastids including proplastids, chloroplasts, chromoplasts, gerontoplasts, leucoplasts, amyloplasts, elaioplasts and etioplasts, preferably chloroplasts. They all have as a common ancestor the aforementioned proplasts.

Unless otherwise specified, the terms “polynucleotides”, “nucleic acid” and “nucleic acid molecule” are interchangeable in the present context. Unless otherwise specified, the terms “peptide”, “polypeptide” and “protein” are interchangeably in the present context. The term “sequence” may relate to polynucleotides, nucleic acids, nucleic acid molecules, peptides, polypeptides and proteins, depending on the context in which the term “sequence” is used. The terms “gene(s)”, “polynucleotide”, “nucleic acid sequence”, “nucleotide sequence”, or “nucleic acid molecule(s)” as used herein refer to a polymeric form of nucleotides of any length, either ribonucleotides or deoxyribonucleotides. The terms refer only to the primary structure of the molecule.

Thus, the terms “gene(s)”, “polynucleotide”, “nucleic acid sequence”, “nucleotide sequence”, or “nucleic acid molecule(s)” as used herein include double- and singlestranded DNA and/or RNA. They also include known types of modifications, for example, methylation, “caps”, substitutions of one or more of the naturally occurring nucleotides with an analog. Preferably, the DNA or RNA sequence comprises a coding sequence encoding the herein defined polypeptide.

A “coding sequence” is a nucleotide sequence, which is transcribed into a RNA, e.g. a regulatory RNA, such as a miRNA, a ta-siRNA, cosuppression molecule, an RNAi, a ribozyme, etc. or preferably into a mRNA which is translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleotide sequences or genomic DNA, while introns may be present as well under certain circumstances. The terms “coding sequence” and “coding region” are interchangeable in the present context.

As used in the present context a nucleic acid molecule may also encompass the untranslated sequence located at the 3′ and/or at the 5′ end of the coding gene region, for example at least 500, preferably 200, especially preferably 100, nucleotides of the sequence upstream of the 5′ end of the coding region and/or at least 100, preferably 50, especially preferably 20, nucleotides of the sequence downstream of the 3′ end of the coding gene region. In the event for example the antisense, RNAi, snRNA, dsRNA, siRNA, miRNA, ta-siRNA, cosuppression molecule, ribozyme etc. technology is used coding regions as well as the 5′- and/or 3′-regions can advantageously be used.

However, it is often advantageous only to choose the coding region of a nucleic acid sequence for cloning and expression purposes. Except as not otherwise specified in the sequence listing according to WI PO Standard 25 for a respective CDS usually the coding region of a nucleic acid molecule is depicted at numeric identifier <222>, whereby the coding region starts at the position given by the first number (given in brackets) and ends at the position given by the second number (given in brackets) and thereafter under numeric identifier <400>the respective sequence is disclosed.

Unless otherwise specified, the term “polypeptide” refers to a polymer of amino acids (amino acid sequence) and does not refer to a specific length of the molecule. Thus, peptides and oligopeptides are included within the definition of polypeptide. This term does also refer to or include post-translational modifications of the polypeptide, for example, glycosylations, acetylations, phosphorylations and the like. Included within the definition are, for example, polypeptides containing one or more analogs of an amino acid (including, for example, unnatural amino acids, etc.), polypeptides with substituted linkages, as well as other modifications known in the art, both naturally occurring and non-naturally occurring. The terms “protein” and “polypeptide” used in this application are interchangeable.

The term “recovering” means the isolation of the fine chemical in different purities, that means on the one hand harvesting of the biological material, which contains the fine chemical without further purification and on the other hand purities of the fine chemical between 5% and 100% purity, preferred purities are in the range of 10% and 99%. In one embodiment, the purities are at least 20%, 30%, 40%, 50%, 60%, 70%, 80%, 90%, 95% or 99%.

A “transit peptide” is an amino acid sequence, whose encoding nucleic acid sequence is translated together with the corresponding structural gene. That means the transit peptide is an integral part of the translated protein and forms an amino terminal extension of the protein. Both are translated as so called “preprotein”. In general the transit peptide is cleaved off from the preprotein during or just after import of the protein into the correct cell organelle such as a plastid or mitochondrion to yield the mature protein. The transit peptide ensures correct localization of the mature protein by facilitating the transport of proteins through intracellular membranes. In principle the nucleic acid sequence encoding a transit peptide can be isolated from every organism such as microorganisms such as algae or plants containing plastids, preferably chloroplasts, or mitochondria. Preferred nucleic acid sequences encoding a transit peptide are derived from a nucleic acid sequence encoding a protein finally resided in the plastid or mitochondria, respectively, and stemming from an organism selected from the group consisting of the genera:

Acetabularia, Arabidopsis, Brassica, Capsicum, Chlamydomonas, Cucurbita, Dunaliella, Euglena, Flaveria, Glycine, Helianthus, Hordeum, Lemna, Lolium, Lycopersion, Malus, Medicago, Mesembryanthemum, Nicotiana, Oenotherea, Oryza, Petunia, Phaseolus, Physcomitrella, Pinus, Pisum, Raphanus, Silene, Sinapis, Solanum, Spinacea, Stevia, Synechococcus, Triticum and Zea.

Advantageously plastidial transit peptides, which are beneficially used in an embodiment of the process of the present invention, are derived from the nucleic acid sequence encoding a protein selected from the group consisting of

ribulose bisphosphate carboxylase/oxygenase, 5-enolpyruvyl-shikimate-3-phosphate synthase, acetolactate synthase, chloroplast ribosomal protein CS17, Cs protein, ferredoxin, plastocyanin, ribulose bisphosphate carboxylase activase, tryptophan synthase, acyl carrier protein, plastid chaperonin-60, cytochrome c552, 22-kDA heat shock protein, 33-kDa Oxygen-evolving enhancer protein 1, ATP synthase γ subunit, ATP synthase δ subunit, chlorophyll-a/b-binding proteinII-1, Oxygen-evolving enhancer protein 2, Oxygen-evolving enhancer protein 3, photosystem I: P21, photosystem I: P28, photosystem I: P30, photosystem I: P35, photosystem I: P37, glycerol-3-phosphate acyltransferases, chlorophyll a/b binding protein, CAB2 protein, hydroxymethyl-bilane synthase, pyruvate-orthophosphate dikinase, CAB3 protein, plastid ferritin, ferritin, early light-inducible protein, glutamate-1-semialdehyde aminotransferase, protochlorophyllide reductase, starch-granule-bound amylase synthase, light-harvesting chlorophyll a/b-binding protein of photosystem II, major pollen allergen Lol p 5a, plastid CIpB ATP-dependent protease, superoxide dismutase, ferredoxin NADP oxidoreductase, 28-kDa ribonucleoprotein, 31-kDa ribonucleoprotein, 33-kDa ribonucleoprotein, acetolactate synthase, ATP synthase CFO subunit 1, ATP synthase CFO subunit 2, ATP synthase CFO subunit 3, ATP synthase CFO subunit 4, cytochrome f, ADP-glucose pyrophosphorylase, glutamine synthase, glutamine synthase 2, carbonic anhydrase, GapA protein, heat-shock-protein hsp21, phosphate translocator, plastid CIpA ATP-dependent protease, plastid ribosomal protein CL24, plastid ribosomal protein CL9, plastid ribosomal protein PsCL18, plastid ribosomal protein PsCL25, DAHP synthase, starch phosphorylase, root acyl carrier protein II, betaine-aldehyde dehydrogenase, GapB protein, glutamine synthetase 2, phosphoribulokinase, nitrite reductase, ribosomal protein L12, ribosomal protein L13, ribosomal protein L21, ribosomal protein L35, ribosomal protein L40, triose phosphate-3-phosphoglyerate-phosphate translocator, ferredoxin-dependent glutamate synthase, glyceraldehyde-3-phosphate dehydrogenase, NADP-dependent malic enzyme and NADP-malate dehydrogenase.

More preferred the nucleic acid sequence encoding a plastidal transit peptide is derived from a nucleic acid sequence encoding a protein finally resided in the plastid and stemming from an organism selected from the group consisting of the species:

Acetabularia mediterranea, Arabidopsis thaliana, Brassica campestris, Brassica napus, Capsicum annuum, Chlamydomonas reinhardtii, Cucurbita moschata, Dunaliella salina, Dunaliella tertiolecta, Euglena gracilis, Flaveria trinervia, Glycine max, Helianthus annuus, Hordeum vulgare, Lemna gibba, Lolium perenne, Lycopersion esculentum, Malus domestica, Medicago falcata, Medicago sativa, Mesembryanthemum crystallinum, Nicotiana plumbaginifolia, Nicotiana sylvestris, Nicotiana tabacum, Oenotherea hookeri, Oryza sativa, Petunia hybrida, Phaseolus vulgaris, Physcomitrella patens, Pinus tunbergii, Pisum sativum, Raphanus sativus, Silene pratensis, Sinapis alba, Solanum tuberosum, Spinacea oleracea, Stevia rebaudiana, Synechococcus, Synechocystis, Triticum aestivum and Zea mays.

Even more preferred nucleic acid sequences are encoding plastidal transit peptides as disclosed by von Heijne et al. (Plant Molecular Biology Reporter, 9 (2), 104 (1991)), which are hereby incorporated by reference. Table a shows some examples of the transit peptide sequences disclosed by von Heijne et al. According to the disclosure of the invention especially in the examples the skilled worker is able to link other nucleic acid sequences disclosed by von Heijne et al. to the respective nucleic acid sequences shown in Table I, columns 5 or 8, preferably the respective coding region thereof, or homologs or fragments thereof. Most preferred nucleic acid sequences encoding transit peptides are derived from the genus Spinacia such as chloroplast 30S ribosomal protein PSrp-1, root acyl carrier protein II, acyl carrier protein, ATP synthase: γ subunit, ATP synthase: δ subunit, cytochrom f, ferredoxin I, ferredoxin NADP oxidoreductase (=FNR), nitrite reductase, phosphoribulokinase, plastocyanin or carbonic anhydrase. The skilled worker will recognize that various other nucleic acid sequences encoding transit peptides can easily isolated from plastid-localized proteins, which are expressed from nuclear genes as precursors and are then targeted to plastids. Such transit peptides encoding sequences can be used for the construction of other expression constructs. The transit peptides advantageously used in the inventive process and which are part of the inventive nucleic acid sequences and proteins are typically 20 to 120 amino acids, preferably 25 to 110, 30 to 100 or 35 to 90 amino acids, more preferably 40 to 85 amino acids and most preferably 45 to 80 amino acids in length and function post-translationally to direct the protein to the plastid, preferably to the chloroplast. The nucleic acid sequences encoding such transit peptides are localized upstream of nucleic acid sequence encoding the mature protein. For the correct molecular joining of the transit peptide encoding nucleic acid and the nucleic acid encoding the protein to be targeted it is sometimes necessary to introduce additional base pairs at the joining position, which form restriction enzyme recognition sequences useful for the molecular joining of the different nucleic acid molecules. This procedure might lead to very few additional amino acids at the Nterminal of the mature imported protein, which usually and preferably do not interfere with the protein function. In any case, the additional base pairs at the joining position which form restriction enzyme recognition sequences have to be chosen with care, in order to avoid the formation of stop codons or codons which encode amino acids with a strong influence on protein folding, like e.g. proline. It is preferred that such additional codons encode small n.d. structural flexible amino acids such as glycine or alanine.

As mentioned above the nucleic acid sequences coding for the proteins as shown in Table II, application no. 7, column 5 or 8, or homologs or fragments thereof, are joined to a nucleic acid sequence encoding a plastidic transit peptide, e.g, if for the nucleic acid molecule in Table I, column 6 the term “plastidic” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide coding sequence is fused in frame to the nucleic acid sequence coding for proteins as shown in Table II, application no. 7, column 5 or 8, or homologs or fragments thereof, e.g., if for the nucleic acid molecule in Table I, column 6 the term “plastidic” is indicated.

Other plastid transit peptides are disclosed by Schmidt et al. (J. Biol. Chem. 268 (36), 27447 (1993)), Della-Cioppa et al. (Plant. Physiol. 84, 965 (1987)), de Castro Silva Filho et al. (Plant Mol. Biol. 30, 769 (1996)), Zhao et al. (J. Biol. Chem. 270 (11), 6081 (1995)), Römer et al. (Biochem. Biophys. Res. Commun., 196 (3), 1414 (1993)), Keegstra et al. (Annu. Rev. Plant Physiol. Plant Mol. Biol. 40, 471 (1989)), Lubben et al. (Photosynthesis Res. 17, 173 (1988)) and Lawrence et al. (J. Biol. Chem. 272, (33), 20357 (1997)), Cho et al. (Planta 224, 598 (2006)), Lucia et al. (Transgenic R. 17 (4), 529 (2008)), Murayama et al. (Planta 225 (5), 1193 (2007)). A general review about targeting is disclosed by Allison R. Kermode in Critical

Reviews in Plant Science 15 (4), 285 (1996).

Favored plastidal transit peptide sequences, which are used in an embodiment of the inventive process and which form part of the inventive nucleic acid sequences are generally enriched in hydroxylated amino acid residues (serine and threonine), with these two residues generally constituting 20-35% of the total. They often have an amino-terminal region empty of Gly, Pro, and charged residues. Furthermore they have a number of small hydrophobic amino acids such as valine and alanine and generally acidic amino acids are lacking. In addition they generally have a middle region rich in Ser, Thr, Lys and Arg. Overall they have very often a net positive charge.

Advantageously mitochondrial transit peptides, which are beneficially used in an embodiment of the process of the present invention, are derived from the nucleic acid sequence encoding a protein selected from the group consisting of 22 kDA heat shock protein; 70 kDA heat shock protein; 83 kDA heat shock protein; 40S ribosomal protein S19; 50S ribosomal protein L15; ribosomal protein L29; 22 kDA PSST protein of complex I; 2-oxoacid dehydrogenase family protein; 2-oxoglutarate/malate translocator; 3-methyl-2-oxobutanoate hydroxymethyltransferase; 3-Methylcrotonyl-coenzyme A carboxylase

(MCCase); 7,8 - Dihydropteroate synthase (DHPS)/6-hydroxymethyl-7,8-dihydropterine pyrophosphokinase (HPPK); aconitate hydratase; acyl carrier protein (ACP); ADP/ATP translocase; alanyl-tRNA synthetase; alcohol dehydrogenase (ADH); alternative oxidase (AOX); aminoacyltRNA ligase; asparate aminotransferase; ATP synthase alpha subunit; ATP synthase beta subunit; ATP synthase delta subunit; ATP synthase epsilon subunit; ATP synthase gamma subunit; ATP-dependent Clp protease - proteolytic subunit; Chaperonin 60—CPN60; Chaperonin 60 (2)—CPN60-2; Chaperonin 60(1) - CPN60-1; citrate synthase; cytochrome b-cl complex subunit Rieske FeS Protein; cytochrome c reductase-processing peptidase subunit II; dihydrolipoamide S-acetyltransferase; farnesyl-diphosphate synthase 1; formate dehydrogenase ; fumarate hydratase; gamma carbonic anhydrase protein (gammaCA); gamma carbonic anhydrase-like protein 1 (gammaCAL1); gamma carbonic anhydrase-like protein 2 (gammaCAL2); gammaaminobutyric acid transaminase (GABA-T); glutathione reductase (GR); glycine decarboxylase subunit H; glycine decarboxylase subunit L; glycine decarboxylase subunit P; glycine decarboxylase subunit T; isovaleryl-CoA dehydrogenase (IVD); lipoamide dehydrogenase; malate oxidoreductase; manganese superoxide dismutase (Mn)SOD; methylmalonate-semialdehyde dehydrogenase; mitochondrial-processing peptidase beta subunit (MPP); mitochondrialprocessing peptidase subunit alpha (MPP); monodehydroascorbate reductase (MDHAR; NAD dependent isocitrate dehydrogenase; NAD dependent malate dehydrogenase; NAD-dependent malic enzyme; NAD-dependent malic enzyme 59 kDa isoform; NAD-dependent malic enzyme 62 kDa isoform; NAD-dependent malic enzyme 65 kDa isoform; NADH ubiquinone oxidoreductase 29 kDa subunit; NADH-ubiquinone oxidoreductase 18 kDa subunit; NADH-ubiquinone oxidoreductase 20 kDa subunit; NADH-ubiquinone oxidoreductase 23 kDa subunit; NADH-ubiquinone oxidoreductase 75 kDa subunit; NADP dependent isocitrate dehydrogenase; NADP dependent malate dehydrogenase; nucleoside diphosphate kinase; nucleoside diphosphate kinase III; o-acetylserine (thiol) lyase (OAS-TL) ; propionyl-CoA carboxylase; protoporphyrinogen IX oxidase; pyruvate dehydrogenase E1 component subunit alpha; serine acetyltransferase (SAT); serine hydroxymethyltransferase; succinate dehydrogenase (SDH); succinic semialdehyde dehydrogenase (SSADH); succinyl-CoA ligase (GDP-forming) alpha-chain; succinyl-CoA ligase [GDP-forming] subunit beta; thiosulfate sulfurtransferase; threonyl-tRNA synthetase; trans-2-enoyl-CoA reductase; translocase inner membrane (TIM); translocase outer membrane (TOM); tRNA synthetase class I and ubiquinol cytochrome C oxidoreductase complex.

More preferred the nucleic acid sequence encoding a mitochondrial transit peptide is derived from a nucleic acid sequence encoding a protein finally resided in the mitochondrion and stemming from an organism selected from the group consisting of the species:

Acetabularia mediterranea, Arabidopsis thaliana, Brassica campestris, Brassica napus, Capsicum annuum, Chlamydomonas reinhardtii, CururbitaCucurbita moschata, Daucus carota, Dunaliella salina, Dunaliella tertiolecta, Euglena gracilis, Flaveria trinervia, Glycine max, Helianthus annuus, Hordeum vulgare, Lactuca sativa, Lemna gibba, Lolium perenne, Lycopersion esculentum, Malus domestica, Medicago falcata, Medicago sativa, Mesembryanthemum crystallinum, Nicotiana plumbaginifolia, Nicotiana sylvestris, Nicotiana tabacum, Oenotherea hookeri, Oryza sativa, Petunia hybrida, Phaseolus vulgaris, Physcomitrella patens, Pinus tunbergii, Pisum sativum, Pyrus pyrifolia, Raphanus sativus, Saccharum officinarum, Silene pratensis, Sinapis alba, Solanum tuberosum, Spinacea oleracea, Stevia rebaudiana, Synechococcus, Synechocystis, Triticum aestivum and Zea mays.

Even more preferred nucleic acid sequences are encoding mitochondrial transit peptides as disclosed by White and Scandalios (Proc. Natl. Acad. Sci., 86 (10), 3534 (1989)), Cho et al (Plant Physiol. 149, 745 (2009)), Chatre et al (Journal of Experimental Botany, 60 (3), 741 (2009)), Murayama et al (Planta 225 (5), 1193 (2007) and Manzano et al (2006) , which are hereby incorporated by reference. Table b shows some examples of the transit peptide sequences disclosed by White and Scandalios (1989), Cho et al (2009), Chatre et al (2009), Murayama et al (2007) and Manzano et al (2006), Glaser et al (Plant Mol Biol. 38 (1-2), 311 (1998) and Huang et al (Plant Phys. 150, 1272 (2009)). According to the disclosure of the invention especially in the examples the skilled worker is able to link other nucleic acid sequences disclosed by Glaser et al (Plant Mol Biol. 38 (1-2), 311 (1998)) and Huang et al (Plant Phys. 150, 1272 (2009)) to the respective nucleic acid sequences shown in Table I, columns 5 or 8, preferably the coding region thereof, or a homolog or a fragment thereof. Most preferred nucleic acid sequences encoding transit peptides are derived from the genus Arabidopsis thaliana such as mitochondrial malate oxidoreductase, manganese superoxide dismutase (Mn)SOD, translocase inner membrane (TIM), translocase outer membrane (TOM), ATP synthase alpha subunit, ATP synthase beta subunit, ATP synthase gamma subunit, ATP synthase delta subunit, ATP synthase epsilon subunit, ADP/ATP translocase, aconitate hydratase and isovaleryl-CoA dehydrogenase (IVD). The skilled worker will recognize that various other nucleic acid sequences encoding transit peptides can easily isolated from mitochondrion-localized proteins, which are expressed from nuclear genes as precursors and are then targeted to mitochondria. Such transit peptides encoding sequences can be used for the construction of other expression constructs. The transit peptides advantageously used in the inventive process and which are part of the inventive nucleic acid sequences and proteins are typically 20 to 120 amino acids, preferably 25 to 110, 30 to 100 or 35 to 90 amino acids, more preferably 40 to 85 amino acids and most preferably 45 to 80 amino acids in length and function post-translationally to direct the protein to the mitochondrion. The nucleic acid sequences encoding such transit peptides are localized upstream of nucleic acid sequence encoding the mature protein. For the correct molecular joining of the transit peptide encoding nucleic acid and the nucleic acid encoding the protein to be targeted it is sometimes necessary to introduce additional base pairs at the joining position, which forms restriction enzyme recognition sequences useful for the molecular joining of the different nucleic acid molecules. This procedure might lead to very few additional amino acids at the N-terminal of the mature imported protein, which usually and preferably do not interfere with the protein function. In any case, the additional base pairs at the joining position which forms restriction enzyme recognition sequences have to be chosen with care, in order to avoid the formation of stop codons or codons which encode amino acids with a strong influence on protein folding, like e.g. proline. It is preferred that such additional codons encode small n.d. structural flexible amino acids such as glycine or alanine.

As mentioned above the nucleic acid sequences coding for the proteins as shown in the respective line in Table II, application no. 7, column 5 or 8, or homologs or fragments thereof, are joined to a nucleic acid sequence encoding a mitochondric transit peptide, e.g., if for the respective nucleic acid molecule in Table I, column 6 the term “mitochondric” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in the respective line in Table II, application no. 7, column 5 or 8, or homologs or fragments thereof, e.g., if for the nucleic acid molecule in the respective line in Table I, column 6 the term “mitochondric” is indicated.

Other mitochondric transit peptides are disclosed by Cho et al. (Plant Physiol. 149, 745 (2009)), Gnanasambandam et al. (Functional Plant Biology 35(2), 166, (2008)), Subbaiah et al. (J. Biol.Chem. 281 (23), 15625 (2006)), Murcha et al. (Plant Physiol. 138, 2134 (2005)), Chatre et al. (Journal of Experimental Botany, 60 (3), 741 (2009)), Millar and Heazlewood (Plant Physiol. 131, 443 (2003)), White and Scandalios (Proc. Natl. Acad. Sci., 86 (10), 3534 (1989)), Laloi et al. (Proc. Natl. Acad. Sci., 98 (24), 14144 (2001)), Barranco-Medina et al. (Plant Physiol. and Biochem. 45, 729 (2007)), Olejnik et al. (FEBS J. 274 (18), 4877 (2007)), Fabre et al. (Plant Cell and Env. 30 (5), 617 (2007)), Murayama et al. (Planta 225 (5), 1193 (2007)), Bathgate et al. (Eur. J. Biochem. 183, 303 (1989)), Winning et al. (Plant J. 2, 763 (1992)), Murcha et al. (Plant Physiol. 143, 199 (2007)), Lehnerer et al. (Botanica Acta, 107, 306 (1994)), Kimura et al. (J. Biol. Chem. 265 (11), 6079 (1990)), Yang et al. (Plant Mol. Biol., 62, 951 (2006)).

Favored mitochondric transit peptide sequences, which are used in an embodiment of the inventive process and which form part of the inventive nucleic acid sequences are generally enriched in hydroxylated amino acid residues (serine and threonine), with these two residues generally constituting 20-35% of the total. They often have an amino-terminal region empty of Gly, Pro, and charged residues. Furthermore they have a number of small hydrophobic amino acids such as valine and alanine and generally acidic amino acids are lacking.

In addition they generally have a middle region rich in Ser, Thr, Lys and Arg. Overall they have very often a net positive charge.

Nucleic acid sequences coding for the transit peptides may be chemically synthesized either in part or wholly according to structure of transit peptide sequences disclosed in the prior art. Said natural or chemically synthesized sequences can be directly linked to the sequences encoding the mature protein or via a linker nucleic acid sequence, which may be typically less than 500 base pairs, preferably less than 450, 400, 350, 300, 250 or 200 base pairs, more preferably less than 150, 100, 90, 80, 70, 60, 50, 40 or 30 base pairs and most preferably less than 25, 20, 15, 12, 9, 6 or 3 base pairs in length and are in frame to the coding sequence. Furthermore favorable nucleic acid sequences encoding transit peptides may comprise sequences derived from more than one biological and/or chemical source and may include a nucleic acid sequence derived from the amino-terminal region of the mature protein, which in its native state is linked to the transit peptide. In a preferred embodiment of the invention said amino-terminal region of the mature protein is typically less than 150 amino acids, preferably less than 140, 130, 120, 110, 100 or 90 amino acids, more preferably less than 80, 70, 60, 50, 40, 35, 30, 25 or 20 amino acids and most preferably less than 19, 18, 17, 16, 15, 14, 13, 12, 11 or 10 amino acids in length. But even shorter or longer stretches are also possible. In addition target sequences, which facilitate the transport of proteins to other cell compartments such as the vacuole, endoplasmic reticulum, golgi complex, glyoxysomes, peroxisomes, mitochondria (in case of a plastidal transit peptide) or plastids, especially chloroplasts (in case of a mitochondric transit peptide) may be also part of the inventive nucleic acid sequence. The proteins translated from said inventive nucleic acid sequences are a kind of fusion proteins that means the nucleic acid sequences encoding the plastidal transit peptide for example the ones shown in Table a, preferably the last one of the Table a are joint to the respective nucleic acid sequences shown in Table I, application no. 7, columns 5 or 8, especially the coding region thereof, or homologs or fragments thereof, in case of nucleic acid sequences being expressed in plastids, and that means the nucleic acid sequences encoding the mitochondric transit peptide for example the ones shown in Table b, preferably the last one of the Table b are joint to the respective nucleic acid sequences shown in Table I, application no. 7, columns 5 or 8, especially the coding region thereof, or homologs or fragments thereof, in case of nucleic acid sequences being expressed in mitochondria. The person skilled in the art is able to join said sequences in a functional manner. Advantageously the transit peptide part is cleaved off from the protein part shown in Table II, application no. 7, columns 5 or 8, or a homolog or fragment thereof, during or after the transport into the organelles, preferably into the plastids or mitochondria, respectively.

All products of the cleavage of the preferred transit peptide shown in the last line of Table a have preferably the N-terminal amino acid sequences QIA CSS or QIA EFQLTT in front of the start methionine of the respective protein mentioned in Table II, application no. 7, column 5 or 8.

or homologs or fragments thereof. Other short amino acid sequences of an range of 1 to 20 amino acids preferable 2 to 15 amino acids, more preferable 3 to 10 amino acids most preferably 4 to 8 amino acids are also possible in front of the start methionine of the protein mentioned in Table II, application no. 7, columns 5 or 8, or homologs or fragments thereof. In case of the amino acid sequence QIA CSS the three amino acids in front of the start methionine are stemming from the LIC (=ligatation independent cloning) cassette. Said short amino acid sequence is preferred in the case of the expression of E. coli genes. In case of the amino acid sequence QIA EFQLTT the six amino acids in front of the start methionine are stemming from the LIC cassette. Said short amino acid sequence is preferred in the case of the expression of S. cerevisiae genes. The skilled worker knowns that other short sequences are also useful in the expression of the respective nucleic acid molecule comprising a polynucleotide as depicted in Table I, application no. 7, column 5 or 8, preferably the coding region thereof, or a homolog or a fragment thereof. Furthermore the skilled worker is aware of the fact that there is not a need for such short sequences in the expression of the genes.

TABLE A
Examples of plastidal transit peptides disclosed by von Heijne et al.

| | | | SEQ | |
|---|---|---|---|---|
| Trans | | | ID | |
| Pep | Organism | Transit Peptide | NO. | Reference |
| 1 | Acetabularia | MASIMMNKSVVLSKECAKPLATPK | 1 | Mol. Gen. Genet. |
| | mediterranea | VTLNKRGFATTIATKNREMMVWQP | | 218, 445(1989) |
| | | FNNKMFETFSFLPP | | |

| | | | | |
|---|---|---|---|---|
| 2 | Arabidopsis | MAASLQSTATFLQSAKIATAPSRG | 2 | EMBO J. 8, 3187 |
| | thaliana | SSHLRSTQAVGKSFGLETSSARLT | | (1989) |
| | | CSFQSDFKDFTGKCSDAVKIAGFA | | |
| | | LATSALVVSGASAEGAPK | | |
| 3 | Arabidopsis | MAQVSRICNGVQNPSLICNLSKSS | 3 | Mol. Gen. Genet. |
| | thaliana | QRKSPLSVSLKTQQHPRAYPISSS | | 210, 437 (1987) |
| | | WGLKKSGMTLIGSELRPLKVMSSV | | |
| | | STAEKASEIVLQPIREISGLIKLP | | |
| 4 | Arabidopsis | MAAATTTTTTSSSISFSTKPSPSS | 4 | Plant Physiol. 85, |
| | thaliana | SKSPLPISRFSLPFSLNPNKSSSS | | 1110 (1987) |
| | | SRRRGIKSSSP SS ISAVLNTTTNV | | |
| | | TTTPSPTKPTKPETF ISRFAPDQP | | |
| | | RKGA | | |
| 5 | Arabidopsis | MITSSLTCSLQALKLSSPFAHGST | 5 | J. Biol. Chem. 265, |
| | thaliana | PLSSLSKPNSFPNHRMPALVPV | | 2763 (1990) |
| 6 | Arabidopsis | MASLLGTSSSAIWASPSLSSPSSK | 6 | EMBO J. 9,1337 |
| | thaliana | PSSSPICFRPGKLFGSKLNAGIQI | | (1990) |
| | | RPKKNRSRYHVSVMNVATEINSTE | | |
| | | QVVGKFDSKKSARPVYPFAAI | | |
| 7 | Arabidopsis | MASTALSSAIVGTSFIRRSPAPISL | 7 | Plant Physiol. 93, |
| | thaliana | RSLPSANTQSLFGLKSGTARGG | | 572 (1990) |
| | | RVVAM | | |
| 8 | Arabidopsis | MAASTMALSSPAFAGKAVNLSPAA | 8 | Nucl. Acids Res. 14, |
| | thaliana | SEVLGSGRVTNRKTV | | 4051 (1986) |
| 9 | Arabidopsis | MAAITSATVTIPSFTGLKLAVSSK | 9 | Gene 65, 59 (1988) |
| | thaliana | PKTLSTISRSSSATRAPPKLALKS | | |
| | | SLKDFGVIAVATAASIVLAGNAMA | | |
| | | MEVLLGSDDGSLAFVPSEFT | | |
| 10 | Arabidopsis | MAAAVSTVGAINRAPLSLNGSGSG | 10 | Nucl. Acids Res. 17, |
| | thaliana | AVSAPASTFLGKKWTVSRFAQSN | | 2871 (1989) |
| | | KKSNGSFKVLAVKEDKQTDGDRWR | | |
| | | GLAYDTSDDQIDI | | |
| 11 | Arabidopsis | MkSSMLSSTAWTSPAQATMVAPF | 11 | Plant Mol. Biol. 11, |
| | thaliana | TGLKSSASFPVTRKANNDITSITS | | 745 (1988) |
| | | NGGRVSC | | |
| 12 | Arabidopsis | MAASGTSATFRASVSSAPSSSSQL | 12 | Proc. Natl. Acad. |
| | thaliana | THLKSPFKAVKY TPLPS SRSKSSS | | Sci. USA 86, 4604 |
| | | FSVSCTIAKDPPVLMAAGSDPALW | | (1989) |
| | | QRPDSFGRFGKFGGKYVPE | | |
| 13 | Brassica | MSTTFCSSVCMQATSLAATTRISF | 13 | Nucl. Acids Res. 15, |
| | campestris | QKPALVSTTNLSFNLRRSIPTRFS | | 7197 (1987) |
| | | ISCAAKPETVEKVSKIVKKQLSLK | | |
| | | DDQKVVAE | | |
| 14 | Brassica | MATTFSASVSMQATSLATTTRISF | 14 | Eur. J. Biochem. |
| | napus | QKPVLVSNHGRTNLSFNLSRTRLSISC | | 174, 287 (1988) |
| 15 | Chlamydomonas | MQALSSRVNIAAKPQRAQRLWRA | 15 | Plant Mol. Biol. 12, |
| | reinhardtii | EEVKAAPKKEVGPKRGSLVK | | 463 (1989) |
| 16 | Cucurbita | MAELIQDKESAQSAATAAAASSGY | 16 | FEBS Lett. 238, 424 |
| | moschata | ERRNEPAHSRKFLEVRSEEELLSCIKK | | (1988) |
| 17 | Spinacea | MSTINGCLTSISPSRTQLKNTSTL | 17 | J. Biol. Chem. 265, |
| | oleracea | RPTFIANSRVNPSSSVPPSLIRNQ | | 5414 (1990) |
| | | PVFAAPAPIITPTL | | |
| 18 | Spinacea | MTTAVTAAVSFPSTKTTSLSARCS | 18 | Curr. Genet. 13, 517 |
| | oleracea | SVISPDKISYKKVPLYYRNVSATG | | (1988) |
| | | KMGPIRAQIASDVEAPPPAPAKVEKMS | | |
| 19 | Spinacea | MTTAVTAAVSFPSTKTTSLSARSS | 19 | |
| | oleracea | SVISPDKISYKKVPLYYRNVSATG | | |
| | | KMGPIRA | | |

TABLE B
Examples of mitochondric transit peptides disclosed by White and
Scandalios (1989); Cho et al (2009); Chatre et al (2008);
Murayama et al (2007); Manzano et al (2006):

| | | | SEQ | |
|---|---|---|---|---|
| Trans | | | ID | |
| Pep | Organism | Transit Peptide | NO. | Reference |
| 20 | Zea mays | MALRTLASKKVLSFPFGGAGRPL | 63 | Proc. Nat. Acad.Sci. |
| | | AAAASARGV | | 86, 3534 (1989) |
| 21 | Oryza | MGKAAAVGTAVVVAAAVGVAVVLA | 64 | Plant Physiol 149, |
| | sativa | | | 745, (2009) |
| 22 | Oryza | MGKGTVVGTAVVVCAAAAAAVGVAVV | 65 | Plant Physiol 149, |
| | sativa | VS | | 745, (2009) |
| 23 | Arabidosis | MIITTHKRDINLLVLQLGAALAV | 66 | J. Exp. Bot. 60 (3), |
| | thaliana | SFAGFLFARFRKNTKR | | 741 (2009) |
| 24 | Oryza | MAAAAISHLRRGAPRHARALYLSTR- | 67 | Planta 225 (5), 1193 |
| | sativa | RFSSSSAAGVAPLAAVAASARR- | | (2007) |
| | | LLSTSVDSGASSGESYKPPLFDPF- | | |
| | | RAASLASSAPPLESPPIEELPDDA- | | |
| | | TPPPEEEPGLPAPEKDPVATACQHE- | | |
| | | LEGLKAVETVRSRKESTEEKEAWSL- | | |
| | | LGRSVVSYCGTA | | |
| 25 | Arabidosis | MSVSCCCRNLGKTIKKAIPSHHLHL- | 68 | Plant Mol. Bio. 61, |
| | thaliana | RSLGGSLYRRRIQSSSMETDLKS | | 195 (2006) |

Alternatively to the targeting of the respective sequences shown in Table II, application no. 7, columns 5 or 8, or homologs or fragments thereof, preferably of sequences in general encoded in the nucleus with the aid of the targeting sequences mentioned for example in Table a alone, Table b alone, or in combination with other targeting sequences preferably into plastids or mitochondria, respectively, the nucleic acids of the invention can directly be introduced into the plastidal or mitochondrial genome. Therefore in a preferred embodiment the respective nucleic acid sequences shown in the respective line in Table I, application no. 7, columns 5 or 8, preferably the coding region thereof, or homologs or fragments thereof, are directly introduced and expressed in plastids e.g., if for the nucleic acid molecule in the respective line in Table I, column 6, the term “plastidic” is indicated, or are directly introduced and expressed in mitochondria e.g., if for the nucleic acid molecule in the respective line in Table I, column 6 the term “mitochondric” is indicated.

The term “introduced” in the context of this specification shall mean the insertion of a nucleic acid sequence into the organism by means of a “transfection”, “transduction” or preferably by “transformation”.

A plastid, such as a chloroplast, or a mitochondrion, has been “transformed” by an exogenous (preferably foreign) nucleic acid sequence if a nucleic acid sequence has been introduced into the plastid or mitochondrion that means that this sequence has crossed the membrane or the membranes of the plastid or of the mitochondrion. The exogenous, preferably foreign, DNA may be integrated (covalently linked) into plastid or mitochondrial DNA making up the genome of the plastid or mitochondrion, or it may remain unintegrated (e.g., by including a chloroplast origin of replication). “Stably” integrated DNA sequences are those, which are inherited through plastid or mitochondrion replication, thereby transferring new plastids or mitochondria, with the features of the integrated DNA sequence to the progeny.

For expression a person skilled in the art is familiar with different methods to introduce the nucleic acid sequences into different organelles, such as the preferred plastids or mitochondria. Such methods are for example disclosed by Pal Maiga (Annu. Rev. Plant Biol. 55, 289, (2004)), Evans T. (WO 2004/040973), McBride K.E. et al. (U.S. Pat. No. 5,455,818), Daniell H. et al. (U.S. Pat. No. 5,932,479 and U.S. Pat. No. 5,693,507) and Straub J. M. et al. (U.S. Pat. No. 6,781,033). A preferred method is the transformation of microspore-derived hypocotyl or cotyledonary tissue (which are green and thus contain numerous plastids) leaf tissue and afterwards the regeneration of shoots from said transformed plant material on selective medium. As methods for the transformation bombarding of the plant material or the use of independently replicating shuttle vectors are well known by the skilled worker. But also a PEG-mediated transformation of plastids or Agrobacterium transformation with binary vectors are possible. Useful markers for the transformation of plastids are positive selection markers for example the chloramphenicol-, streptomycin-, kanamycin-, neomycin-, amikamycin-, spectinomycin-, triazine- and/or lincomycin-resistance genes. As additional markers named in the literature often as secondary markers, genes coding for the resistance against herbicides such as phosphinothricin (=glufosinate, BASTA™, Liberty™, encoded by the bar gene), glyphosate (=N-(phosphonomethyl)glycine, Roundup Ready™, encoded by the 5-enolpyruvylshikimaete-3-phosphate synthase gene =epsps), sulfonylurea (e.g. =Staple™, encoded by the acetolactate synthase gene), imidazolinone (=IMI, e.g. imazethapyr, imazamox, Clearfield™, encoded by the acetohydroxyacid synthase (AHAS) gene, also known as acetolactate synthase (ALS) gene) or bromoxynil (=Buctril™, encoded by the oxy gene) or genes coding for antibiotics such as hygromycin or G418 are useful for further selection. Such secondary markers are useful in the case when most genome copies are transformed. In addition negative selection markers such as the bacterial cytosine deaminase (encoded by the codA gene) are also useful for the transformation of organelles, especially plastids.

Thus, in one embodiment, an activity disclosed herein as being conferred by a polypeptide shown in the respective line in Table II, or a homolog or a fragment thereof, is increased or generated by linking the polypeptide disclosed in respective line in Table II, or a homolog or a fragment thereof, or a polypeptide conferring the same said activity with a targeting signal as herein described, if in the respective line in column 6 of Table II the term “plastidic” is listed for said polypeptide. For example, the polypeptide described can be linked to the targeting signal shown in Table a.

Accordingly, in the method of the invention for producing a transgenic plant with increased fine chemical production as compared to a corresponding, e.g. non-transformed, wild type plant, comprising transforming a non-human organism, preferably a plant cell, a plant or a part thereof, with the mentioned nucleic acid molecule, said nucleic acid molecule selected from said mentioned group encodes for a polypeptide conferring said activity being linked to a targeting signal as mentioned herein, e.g. as mentioned in Table a, e.g. if in the respective line in column 6 of Table II the term “plastidic” is listed for the encoded polypeptide.

To increase the possibility of identification of transformants it is possible to use reporter genes other then the aforementioned resistance genes or in addition to said genes. Reporter genes are for example [3-galactosidase-, [3-glucuronidase-(GUS), alkaline phosphatase- and/or green-fluorescent protein-genes (GFP).

For the process of the present invention it may be of great advantage that by transforming the plastids the intraspecies specific transgene flow is blocked, because a lot of species such as corn, cotton and rice have a strict maternal inheritance of plastids. By placing the respective genes specified in the respective line in Table I, application no. 7, columns 5 or 8, preferably the coding region, or homologs or fragments thereof, e.g. if for the nucleic acid molecule in the respective line in column 6 of Table I the term “plastidic” is indicated, in the plastids of plants, these genes will not be present in the pollen of said plants.

A further preferred embodiment of the invention relates to the use of so called “chloroplast localization sequences”, in which a first RNA sequence or molecule is capable of transporting or “chaperoning” a second RNA sequence, such as a RNA sequence transcribed from the sequences depicted in the respective line in Table I, application no. 7, columns 5 or 8, preferably the coding region thereof, or a homolog or a fragment thereof, or a sequence encoding a protein, as depicted in the respective line in Table II, application no. 7, columns 5 and 8, or a homolog or a fragment thereof, from an external environment inside a cell or outside a plastid into a chloroplast. In one embodiment the chloroplast localization signal is substantially similar or complementary to a complete or intact viroid sequence. The chloroplast localization signal may be encoded by a DNA sequence, which is transcribed into the chloroplast localization RNA. The term “viroid” refers to a naturally occurring single stranded RNA molecule (Flores, C. R. Acad. Sci. III. 324 (10), 943 (2001)). Viroids usually contain about 200-500 nucleotides and generally exist as circular molecules. Examples of viroids that contain chloroplast localization signals include but are not limited to ASBVd, PLMVd, CChMVd and ELVd. The viroid sequence or a functional part of it can be fused to the sequences depicted in the respective line in Table I, application no. 7, columns 5 or 8, preferably the coding region thereof, or a homolog or a fragment thereof, or a sequence encoding a protein, as depicted in the respective line in Table II, application no. 7, columns 5 or 8, or a homolog or a fragment thereof, in such a manner that the viroid sequence transports a sequence transcribed from a sequence as depicted in the respective line in Table I, application no. 7 columns 5 or 8, preferably the coding region thereof, or a homolog or a fragment thereof, or a sequence encoding a protein as depicted in the respective line in Table II, application no. 7 columns 5 or 8, or a homolog or a fragment thereof, into the chloroplasts, e.g. if for the nucleic acid molecule in column 6 of Table I the term “plastidic” is indicated. A preferred embodiment uses a modified ASBVd (Navarro et al., Virology. 268 (1), 218 (2000)).

In a further specific embodiment the protein to be expressed in the plastids such as the proteins depicted in the respective line in Table II, application no. 7, columns 5 or 8, or a homolog or a fragment thereof, e.g. if for the nucleic acid molecule in the respective line in column 6 of Table I the term “plastidic” is indicated, are encoded by different nucleic acids. Such a method is disclosed in WO 2004/040973, which shall be incorporated by reference. WO 2004/040973 teaches a method, which relates to the translocation of an RNA corresponding to a gene or gene fragment into the chloroplast by means of a chloroplast localization sequence. The genes, which should be expressed in the plant or plant cells, are split into nucleic acid fragments, which are introduced into different compartments in the plant e.g. the nucleus, the plastids and/or mitochondria. Additionally plant cells are described in which the chloroplast contains a ribozyme fused at one end to an RNA encoding a fragment of a protein used in the inventive process such that the ribozyme can trans-splice the translocated fusion RNA to the RNA encoding the gene fragment to form and as the case may be reunite the nucleic acid fragments to an intact mRNA encoding a functional protein for example as disclosed in the respective line in Table II, application no. 7, columns 5 or 8, or a homolog or a fragment thereof.

In a preferred embodiment of the invention the nucleic acid sequences as shown in the respective line in Table I, application no. 7, columns 5 or 8, preferably the coding region thereof, or homologs or fragments thereof, e.g. if for the nucleic acid molecule in the respective line in column 6 of Table I the term “plastidic” is indicated, used in the inventive process are transformed into plastids, which are metabolically active. Those plastids should preferably maintain at a high copy number in the plant or plant tissue of interest, most preferably the chloroplasts found in green plant tissues, such as leaves or cotyledons or in seeds.

For a good expression in the plastids the nucleic acid sequences as shown in the respective line in Table I, application no. 7, columns 5 or 8, preferably the coding region thereof, or homologs or fragments thereof, e.g. if for the nucleic acid molecule in the respective line in column 6 of Table I the term “plastidic” is indicated, are introduced into an expression cassette using preferably a promoter and terminater, which are active in plastids preferably a chloroplast promoter. Examples of such promoters include the psbA promoter from the gene from spinach or pea, the rbcL promoter, and the atpB promoter from corn.

In another embodiment, an activity disclosed herein as being conferred by a polypeptide shown in the respective line in Table II is increased or generated by linking the polypeptide disclosed in the respective line in Table II or a polypeptide conferring the same said activity with an targeting signal as herein described, if in the respective line in column 6 of Table II the term “mitochondric” is listed for said polypeptide. For example, the polypeptide described can be linked to the targeting signal shown in Table b.

In these cases the respective organelle is a mitochondrion. However, the remarks made regarding the plastids are applicable in analogy.

In another embodiment, an activity disclosed herein as being conferred by a polypeptide shown in Table II is increased or generated by the polypeptide disclosed in the respective line in Table II or a polypeptide conferring the same said activity without linking said polypeptide shown in the respective line in Table II to a targeting signal, if in the respective line in column 6 of Table II the term “non-targeted” is listed for said polypeptide.

For the purposes of the description of the present invention, the terms “non-targeted” shall indicate, that the nucleic acid of the invention is expressed without the addition of a “non-natural transit peptide encoding sequence”. A “non-natural transit peptide encoding sequence” is a sequence which is not a natural part of a nucleic acid of the invention, e.g. of the nucleic acids depicted in the respective line in Table I column 5 or 8, preferably the coding region thereof, or homologs or fragments thereof, but which is rather added by molecular manipulation steps as for example described in the example under “plastid targeted expression”. However, the terms “non-targeted” shall not exclude a targeted localization to any cell compartment for the products of the inventive nucleic acid sequences by their naturally occurring sequence properties within the background of the transgenic organism. The subcellular location of the mature polypeptide derived from the enclosed sequences can be predicted by a skilled person for the organism (plant) by using software tools like TargetP (Emanuelsson et al., J. Mol. Biol. 300, 1005 (2000)), ChloroP (Emanuelsson et al., Protein Science 8, 978 (1999)) or other predictive software tools (Emanuelsson et al., Nature Protocols 2, 953 (2007)).

Preferably, the process according to the invention further comprises the step of recovering the fine chemical, which is synthesized by the organism from the organism and/or from the culture medium used for the growth or maintenance of the organism. The term “recovering” means the isolation of the fine chemical in different purities, that means on the one hand harvesting of the biological material, which contains the fine chemical without further purification and on the other hand purities of the fine chemical between 5% and 100% purity, preferred purities are in the range of 10% and 99%. In one embodiment, the purities are at least 20%, 30%, 40%, 50%, 60%, 70%, 80%, 90%, 95% or 99%.

Advantageously the process for the production of the fine chemical alpha-tocopherol, and/or gamma-tocopherol leads to an enhanced production of the respective fine chemical. The terms “enhanced” or “increase” mean at least a 10%, 20%, 30%, 40% or 50%, preferably at least 60%, 70%, 80%, 90% or 100%, more preferably 150%, 200%, 300%, 400% or 500% higher production of the respective fine chemical alpha-tocopherol, and/or gamma-tocopherol in comparison to the wild-type as defined above, e.g. that means in comparison to a non-human organism without the aforementioned modification of the activity of a protein as shown in the respective line in Table II, application no. 7, column 5 or 8, or a fragment or a homolog thereof. The modification of the activity of a protein as shown in the respective line in Table II, application no. 7, column 5 or 8, or a homolog or a fragment thereof, or their combination can be achieved by joining the protein to a respective transit peptide, e.g. if for the respective encoding nucleic acid molecule in column 6 of Table I the term “plastidic” or “mitochondric” is indicated.

Surprisingly it was found, that the transgenic expression of a protein as shown in the respective line in Table II, application no. 7, column 5, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, conferred a production of or an increase in alpha-tocopherol, and/or gamma-tocopherol, respectively, to the transgenic non-human organism as compared to a corresponding non-transformed wild type.

In one embodiment the transgenic expression of a protein as shown in the respective line in Table II, application no. 7, column 5 or 8, or a homolog or a fragment thereof, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, confers a production of or an increase in alpha-tocopherol, and/or gamma-tocopherol,respectively, to the transgenic non-human organism as compared to a corresponding non-transformed wild type.

Accordingly, in one embodiment, an increase of alpha-tocopherol in a non-human organism, as compared to a corresponding non-transformed wild type non-human organism, is conferred in the process of the invention, if the activity of a polypeptide showing the activity of a pyruvate kinase, or if the activity of the polypeptide 51340801_CANOLA, preferably represented by SEQ ID NO. 15533, or a homolog or fragment thereof, or if the activity of a polypeptide encoded by a nucleic acid molecule comprising the nucleic acid SEQ ID NO. 15532, preferably the coding region thereof, or a homolog or fragment thereof, e.g. derived from Brassica napus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 15532 or polypeptide SEQ ID NO. 15533, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line disclose in column 7 the fine chemical alpha-tocopherol. For example, an increase of the alpha-tocopherol of at least 1 percent, particularly in a range of 45 to 65-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a pyruvate kinase, or if the activity of the polypeptide 51340801_CANOLA, preferably represented by SEQ ID NO. 15533, or a homolog or fragment thereof, or if the activity of a polypeptide encoded by a nucleic acid molecule comprising the nucleic acid SEQ ID NO. 15532, preferably the coding region thereof, or a homolog or fragment thereof, e.g. derived from Brassica napus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 15532 or polypeptide SEQ ID NO. 15533, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line disclose in column 7 the fine chemical gamma-tocopherol. For example, an increase of the gamma-tocopherol of at least 1 percent, particularly in a range of 38 to 134-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a glucose-6-phosphate 1-dehydrogenase, or if the activity of the polypeptide 59582753_SOYBEAN, preferably represented by SEQ ID NO. 16884, or a homolog or fragment thereof, or if the activity of a polypeptide encoded by a nucleic acid molecule comprising the nucleic acid SEQ ID NO. 16883, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 16883 or polypeptide SEQ ID NO. 16884, respectively, or a homolog or a fragment thereof, is increased or generated, or if the activity glucose-6-phosphate 1-dehydrogenase is increased or generated in a non-human organism, like a microorganism or a plant cell, plant or part thereof, especially with plastidic localization, whereby the respective line disclose in column 7 the fine chemical alpha-tocopherol. For example, an increase of the alpha-tocopherol of at least 1 percent, particularly in a range of 50 to 120-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a At1g29350-protein, or if the activity of the polypeptide At1g29350, preferably represented by SEQ ID NO. 17902, or a homolog or fragment thereof, or if the activity of a polypeptide encoded by a nucleic acid molecule comprising the nucleic acid SEQ ID NO. 17901, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 17901 or polypeptide SEQ ID NO. 17902, respectively, or a homolog or a fragment thereof, is increased or generated, or if the activity At1g29350-protein is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 40 to 88-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a transcriptional regulator, or if the activity of the polypeptide At1g43850, preferably represented by SEQ ID NO. 18071, or a homolog or fragment thereof, or if the activity of a polypeptide encoded by a nucleic acid molecule comprising the nucleic acid SEQ ID NO. 18070, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 18070 or polypeptide SEQ ID NO. 18071, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 55 to 134-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a protein kinase, or if the activity of the polypeptide At1g48260, preferably represented by SEQ ID NO. 18236, or a homolog or fragment thereof, or if the activity of a polypeptide encoded by a nucleic acid molecule comprising the nucleic acid SEQ ID NO. 18235, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 18235 or polypeptide SEQ ID NO. 18236, respectively, or a homolog or a fragment thereof, is increased or generated, or if the activity protein kinase is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 66 to 160-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a calcium-dependent protein kinase, or if the activity of the polypeptide At1g61950, preferably represented by SEQ ID NO. 18870, or a homolog or fragment thereof, or if the activity of a polypeptide encoded by a nucleic acid molecule comprising the nucleic acid SEQ ID NO. 18869, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 18869 or polypeptide SEQ ID NO. 18870, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 44 to 91-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a protein phosphatase, or if the activity of the polypeptide At1g72770, preferably represented by SEQ ID NO. 19420, or a homolog or fragment thereof, or if the activity of a polypeptide encoded by a nucleic acid molecule comprising the nucleic acid SEQ ID NO. 19419, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 19419 or polypeptide SEQ ID NO. 19420, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 63 to 133 -percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a MADS box protein transcription factor, or if the activity of the polypeptide At2g42830, preferably represented by SEQ ID NO. 108728, or a homolog or fragment thereof, or if the activity of a polypeptide encoded by a nucleic acid molecule comprising the nucleic acid SEQ ID NO. 108727, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 108727 or polypeptide SEQ ID NO. 108728, respectively, or a homolog or a fragment thereof, is increased or generated, or if the activity MADS box protein transcription factor is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 36 to 55-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a glutaredoxin, or if the activity of the polypeptide At2g47880, preferably represented by SEQ ID NO. 21160, or a homolog or fragment thereof, or if the activity of a polypeptide encoded by a nucleic acid molecule comprising the nucleic acid SEQ ID NO. 21159, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 21159 or polypeptide SEQ ID NO. 21160, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 87 to 232-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a pyruvate kinase, or if the activity of the polypeptide At3g04050, preferably represented by SEQ ID NO. 21498, or a homolog or fragment thereof, or if the activity of a polypeptide encoded by a nucleic acid molecule comprising the nucleic acid SEQ ID NO. 21497, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 21497 or polypeptide SEQ ID NO. 21498, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line disclose in column 7 the fine chemical gamma-tocopherol. For example, an increase of the gamma-tocopherol of at least 1 percent, particularly in a range of 74 to 160-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a ankyrin repeat family protein, or if the activity of the polypeptide At3g04710, preferably represented by SEQ ID NO. 21903, or a homolog or fragment thereof, or if the activity of a polypeptide encoded by a nucleic acid molecule comprising the nucleic acid SEQ ID NO. 21902, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 21902 or polypeptide SEQ ID NO. 21903, respectively, or a homolog or a fragment thereof, is increased or generated, or if the activity ankyrin repeat family protein is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 39 to 64-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a protein phosphatase, or if the activity of the polypeptide At3g06270, preferably represented by SEQ ID NO. 22016, or a homolog or fragment thereof, or if the activity of a polypeptide encoded by a nucleic acid molecule comprising the nucleic acid SEQ ID NO. 22015, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 22015 or polypeptide SEQ ID NO. 22016, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 56 to 56-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a thioredoxin family protein, or if the activity of the polypeptide At3g08710, preferably represented by SEQ ID NO. 22250, or a homolog or fragment thereof, or if the activity of a polypeptide encoded by a nucleic acid molecule comprising the nucleic acid SEQ ID NO. 22249, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 22249 or polypeptide SEQ ID NO. 22250, respectively, or a homolog or a fragment thereof, is increased or generated, or if the activity thioredoxin family protein is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 57 to 140-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a harpininduced family protein, or if the activity of the polypeptide At3g11650, preferably represented by SEQ ID NO. 22612, or a homolog or fragment thereof, or if the activity of a polypeptide encoded by a nucleic acid molecule comprising the nucleic acid SEQ ID NO. 22611, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 22611 or polypeptide SEQ ID NO. 22612, respectively, or a homolog or a fragment thereof, is increased or generated, or if the activity harpin-induced family protein is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 40 to 67-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a harpininduced family protein, or if the activity of the polypeptide At3g11650, preferably represented by SEQ ID NO. 22612, or a homolog or fragment thereof, or if the activity of a polypeptide encoded by a nucleic acid molecule comprising the nucleic acid SEQ ID NO. 22611, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 22611 or polypeptide SEQ ID NO. 22612, respectively, or a homolog or a fragment thereof, is increased or generated, or if the activity harpin-induced family protein is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 43 to 300-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a protein kinase, or if the activity of the polypeptide At3g23000, preferably represented by SEQ ID NO. 1816, or a homolog or fragment thereof, or if the activity of a polypeptide encoded by a nucleic acid molecule comprising the nucleic acid SEQ ID NO. 1815, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 1815 or polypeptide SEQ ID NO. 1816, respectively, or a homolog or a fragment thereof, is increased or generated, or if the activity protein kinase is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 60 to 223-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a monthiol glutaredoxin, or if the activity of the polypeptide At3g62930, preferably represented by SEQ ID NO. 2574, or a homolog or fragment thereof, or if the activity of a polypeptide encoded by a nucleic acid molecule comprising the nucleic acid SEQ ID NO. 2573, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 2573 or polypeptide SEQ ID NO. 2574, respectively, or a homolog or a fragment thereof, is increased or generated, or if the activity monthiol glutaredoxin is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 63 to 110-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 77 to 155-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a transcription factor, or if the activity of the polypeptide At4g09960, preferably represented by SEQ ID NO. 109718, or a homolog or fragment thereof, or if the activity of a polypeptide encoded by a nucleic acid molecule comprising the nucleic acid SEQ ID NO. 109717, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 109717 or polypeptide SEQ ID NO. 109718, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 59 to 154-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 50 to 139 -percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 38 to 81-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a monothiol glutaredoxin, or if the activity of the polypeptide At4g15670, preferably represented by SEQ ID NO. 3280, or a homolog or fragment thereof, or if the activity of a polypeptide encoded by a nucleic acid molecule comprising the nucleic acid SEQ ID NO. 3279, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 3279 or polypeptide SEQ ID NO. 3280, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 53 to 364 -percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a monothiol glutaredoxin, or if the activity of the polypeptide At4g15690, preferably represented by SEQ ID NO. 23845, or a homolog or fragment thereof, or if the activity of a polypeptide encoded by a nucleic acid molecule comprising the nucleic acid SEQ ID NO. 23844, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 23844 or polypeptide SEQ ID NO. 23845, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 53 to 125-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a protein phosphatase, or if the activity of the polypeptide At4g26080, preferably represented by SEQ ID NO. 68850, or a homolog or fragment thereof, or if the activity of a polypeptide encoded by a nucleic acid molecule comprising the nucleic acid SEQ ID NO. 68849, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 68849 or polypeptide SEQ ID NO. 68850, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 54 to 99-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a cyclin D , or if the activity of the polypeptide At4g34160, preferably represented by SEQ ID NO. 24312, or a homolog or fragment thereof, or if the activity of a polypeptide encoded by a nucleic acid molecule comprising the nucleic acid SEQ ID NO. 24311, preferably the coding region thereof, or a homolog or fragment thereof, e.g. derived from Arabidopsis thaliana, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 24311 or polypeptide SEQ ID NO. 24312, respectively, or a homolog or a fragment thereof, is increased or generated, or if the activity cyclin D is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 51 to 200-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 91 to 195-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 69 to 192-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a RNA-binding protein, or if the activity of the polypeptide At5g60110, preferably represented by SEQ ID NO. 70007, or a homolog or fragment thereof, or if the activity of a polypeptide encoded by a nucleic acid molecule comprising the nucleic acid SEQ ID NO. 70006, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 70006 or polypeptide SEQ ID NO. 70007, respectively, or a homolog or a fragment thereof, is increased or generated, or if the activity RNA-binding protein is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 58 to 69-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a RNAbinding protein, or if the activity of the polypeptide At5g60110, preferably represented by SEQ ID NO. 70007, or a homolog or fragment thereof, or if the activity of a polypeptide encoded by a nucleic acid molecule comprising the nucleic acid SEQ ID NO. 70006, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 70006 or polypeptide SEQ ID NO. 70007, respectively, or a homolog or a fragment thereof, is increased or generated, or if the activity RNA-binding protein is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 48 to 108-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a zinc finger protein, or if the activity of the polypeptide At5g64920, preferably represented by SEQ ID NO. 5494, or a homolog or fragment thereof, or if the activity of a polypeptide encoded by a nucleic acid molecule comprising the nucleic acid SEQ ID NO. 5493, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 5493 or polypeptide SEQ ID NO. 5494, respectively, or a homolog or a fragment thereof, is increased or generated, or if the activity zinc finger protein is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 42 to 75-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a NAD-dependent epimerase/dehydratase, or if the activity of the polypeptide AvinDRAFT_{—}3107, preferably represented by SEQ ID NO. 110652, or a homolog or fragment thereof, or if the activity of a polypeptide encoded by a nucleic acid molecule comprising the nucleic acid SEQ ID NO. 110651, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 110651 or polypeptide SEQ ID NO. 110652, respectively, or a homolog or a fragment thereof, is increased or generated, or if the activity NAD-dependent epimerase/dehydratase is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 39 to 66-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a transcriptional regulator, or if the activity of the polypeptide AvinDRAFT_{—}3209, preferably represented by SEQ ID NO. 29287, or a homolog or fragment thereof, or if the activity of a polypeptide encoded by a nucleic acid molecule comprising the nucleic acid SEQ ID NO. 29286, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 29286 or polypeptide SEQ ID NO. 29287, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 53 to 89-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a ThiF family protein, or if the activity of the polypeptide AvinDRAFT_{—}3577, preferably represented by SEQ ID NO. 111156, or a homolog or fragment thereof, or if the activity of a polypeptide encoded by a nucleic acid molecule comprising the nucleic acid SEQ ID NO. 111155, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 111155 or polypeptide SEQ ID NO. 111156, respectively, or a homolog or a fragment thereof, is increased or generated, or if the activity ThiF family protein is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 37 to 38-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a isopropylmalate isomerase large subunit, or if the activity of the polypeptide AvinDRAFT_{—}4847, preferably represented by SEQ ID NO. 102942, or a homolog or fragment thereof, or if the activity of a polypeptide encoded by a nucleic acid molecule comprising the nucleic acid SEQ ID NO. 102941, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 102941 or polypeptide SEQ ID NO. 102942, respectively, or a homolog or a fragment thereof, is increased or generated, or if the activity isopropylmalate isomerase large subunit is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 51 to 68-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a hydrolase, or if the activity of the polypeptide AvinDRAFT_{—}5103, preferably represented by SEQ ID NO. 6511, or a homolog or fragment thereof, or if the activity of a polypeptide encoded by a nucleic acid molecule comprising the nucleic acid SEQ ID NO. 6510, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 6510 or polypeptide SEQ ID NO. 6511, respectively, or a homolog or a fragment thereof, is increased or generated, or if the activity hydrolase is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 145 to 201-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a hydrolase, or if the activity of the polypeptide AvinDRAFT_{—}5103, preferably represented by SEQ ID NO. 6511, or a homolog or fragment thereof, or if the activity of a polypeptide encoded by a nucleic acid molecule comprising the nucleic acid SEQ ID NO. 6510, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 6510 or polypeptide SEQ ID NO. 6511, respectively, or a homolog or a fragment thereof, is increased or generated, or if the activity hydrolase is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 40 to 237-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a electron transfer flavoprotein subunit beta, or if the activity of the polypeptide AvinDRAFT_{—}6679, preferably represented by SEQ ID NO. 74730, or a homolog or fragment thereof, or if the activity of a polypeptide encoded by a nucleic acid molecule comprising the nucleic acid SEQ ID NO. 74729, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 74729 or polypeptide SEQ ID NO. 74730, respectively, or a homolog or a fragment thereof, is increased or generated, or if the activity electron transfer flavoprotein subunit beta is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 44 to 243-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a threonine synthase, or if the activity of the polypeptide B0004, preferably represented by SEQ ID NO. 34890, or a homolog or fragment thereof, or if the activity of a polypeptide encoded by a nucleic acid molecule comprising the nucleic acid SEQ ID NO. 34889, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 34889 or polypeptide SEQ ID NO. 34890, respectively, or a homolog or a fragment thereof, is increased or generated, or if the activity threonine synthase is increased or generated in a non-human organism, like a microorganism or a plant cell, plant or part thereof, especially with plastidic localization, whereby the respective line disclose in column 7 the fine chemical gamma-tocopherol. For example, an increase of the gamma-tocopherol of at least 1 percent, particularly in a range of 75 to 443-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a glucose dehydrogenase, or if the activity of the polypeptide B0124, preferably represented by SEQ ID NO. 35483, or a homolog or fragment thereof, or if the activity of a polypeptide encoded by a nucleic acid molecule comprising the nucleic acid SEQ ID NO. 35482, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 35482 or polypeptide SEQ ID NO. 35483, respectively, or a homolog or a fragment thereof, is increased or generated, or if the activity glucose dehydrogenase is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 38 to 103 -percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a glucose dehydrogenase, or if the activity of the polypeptide B0124, preferably represented by SEQ ID NO. 35483, or a homolog or fragment thereof, or if the activity of a polypeptide encoded by a nucleic acid molecule comprising the nucleic acid SEQ ID NO. 35482, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 35482 or polypeptide SEQ ID NO. 35483, respectively, or a homolog or a fragment thereof, is increased or generated, or if the activity glucose dehydrogenase is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 175 to 277-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a serine protease, or if the activity of the polypeptide B0161, preferably represented by SEQ ID NO. 7082, or a homolog or fragment thereof, or if the activity of a polypeptide encoded by a nucleic acid molecule comprising the nucleic acid SEQ ID NO. 7081, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 7081 or polypeptide SEQ ID NO. 7082, respectively, or a homolog or a fragment thereof, is increased or generated, or if the activity serine protease is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 98 to 238-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a geranyltranstransferase, or if the activity of the polypeptide B0421, preferably represented by SEQ ID NO. 111341, or a homolog or fragment thereof, or if the activity of a polypeptide encoded by a nucleic acid molecule comprising the nucleic acid SEQ ID NO. 111340, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 111340 or polypeptide SEQ ID NO. 111341, respectively, or a homolog or a fragment thereof, is increased or generated, or if the activity geranyltranstransferase is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 42 to 44-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a ATP-binding component of a transport system, or if the activity of the polypeptide B0449, preferably represented by SEQ ID NO. 7334, or a homolog or fragment thereof, or if the activity of a polypeptide encoded by a nucleic acid molecule comprising the nucleic acid SEQ ID NO. 7333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 7333 or polypeptide SEQ ID NO. 7334, respectively, or a homolog or a fragment thereof, is increased or generated, or if the activity ATP-binding component of a transport system is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 42 to 248-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a 3-deoxy-D-arabinoheptulosonate-7-phosphatesynthase, or if the activity of the polypeptide B0754, preferably represented by SEQ ID NO. 75287, or a homolog or fragment thereof, or if the activity of a polypeptide encoded by a nucleic acid molecule comprising the nucleic acid SEQ ID NO. 75286, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 75286 or polypeptide SEQ ID NO. 75287, respectively, or a homolog or a fragment thereof, is increased or generated, or if the activity 3-deoxy-D-arabinoheptulosonate-7-phosphatesynthase is increased or generated in a non-human organism, like a microorganism or a plant cell, plant or part thereof, especially with plastidic localization, whereby the respective line disclose in column 7 the fine chemical alpha-tocopherol. For example, an increase of the alpha-tocopherol of at least 1 percent, particularly in a range of 35 to 68-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a major facilitator superfamily transporter protein, or if the activity of the polypeptide B0898, preferably represented by SEQ ID NO. 7918, or a homolog or fragment thereof, or if the activity of a polypeptide encoded by a nucleic acid molecule comprising the nucleic acid SEQ ID NO. 7917, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 7917 or polypeptide SEQ ID NO. 7918, respectively, or a homolog or a fragment thereof, is increased or generated, or if the activity major facilitator superfamily transporter protein is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 76 to 379-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a b1163-protein, or if the activity of the polypeptide B1163, preferably represented by SEQ ID NO. 37395, or a homolog or fragment thereof, or if the activity of a polypeptide encoded by a nucleic acid molecule comprising the nucleic acid SEQ ID NO. 37394, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 37394 or polypeptide SEQ ID NO. 37395, respectively, or a homolog or a fragment thereof, is increased or generated, or if the activity b1163-protein is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 52 to 56-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a DNA polymerase subunit, or if the activity of the polypeptide B1183, preferably represented by SEQ ID NO. 111911, or a homolog or fragment thereof, or if the activity of a polypeptide encoded by a nucleic acid molecule comprising the nucleic acid SEQ ID NO. 111910, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 111910 or polypeptide SEQ ID NO. 111911, respectively, or a homolog or a fragment thereof, is increased or generated, or if the activity DNA polymerase subunit is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 33 to 45-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a b1219-protein, or if the activity of the polypeptide B1219, preferably represented by SEQ ID NO. 112073, or a homolog or fragment thereof, or if the activity of a polypeptide encoded by a nucleic acid molecule comprising the nucleic acid SEQ ID NO. 112072, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 112072 or polypeptide SEQ ID NO. 112073, respectively, or a homolog or a fragment thereof, is increased or generated, or if the activity b1219-protein is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 51 to 98-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a b1234-protein, or if the activity of the polypeptide B1234, preferably represented by SEQ ID NO. 112112, or a homolog or fragment thereof, or if the activity of a polypeptide encoded by a nucleic acid molecule comprising the nucleic acid SEQ ID NO. 112111, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 112111 or polypeptide SEQ ID NO. 112112, respectively, or a homolog or a fragment thereof, is increased or generated, or if the activity b1234-protein is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 33 to 57-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a NADP-dependent malic enzyme, or if the activity of the polypeptide B1479, preferably represented by SEQ ID NO. 77775, or a homolog or fragment thereof, or if the activity of a polypeptide encoded by a nucleic acid molecule comprising the nucleic acid SEQ ID NO. 77774, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 77774 or polypeptide SEQ ID NO. 77775, respectively, or a homolog or a fragment thereof, is increased or generated, or if the activity NADP-dependent malic enzyme is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 33 to 79-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a b1522-protein, or if the activity of the polypeptide B1522, preferably represented by SEQ ID NO. 7948, or a homolog or fragment thereof, or if the activity of a polypeptide encoded by a nucleic acid molecule comprising the nucleic acid SEQ ID NO. 7947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 7947 or polypeptide SEQ ID NO. 7948, respectively, or a homolog or a fragment thereof, is increased or generated, or if the activity b1522-protein is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 51 to 301-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a acid shock protein, or if the activity of the polypeptide B1597, preferably represented by SEQ ID NO. 38301, or a homolog or fragment thereof, or if the activity of a polypeptide encoded by a nucleic acid molecule comprising the nucleic acid SEQ ID NO. 38300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 38300 or polypeptide SEQ ID NO. 38301, respectively, or a homolog or a fragment thereof, is increased or generated, or if the activity acid shock protein is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 50 to 302-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a transport protein, or if the activity of the polypeptide B1601, preferably represented by SEQ ID NO. 7993, or a homolog or fragment thereof, or if the activity of a polypeptide encoded by a nucleic acid molecule comprising the nucleic acid SEQ ID NO. 7992, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 7992 or polypeptide SEQ ID NO. 7993, respectively, or a homolog or a fragment thereof, is increased or generated, or if the activity transport protein is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 52 to 112-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a trehalosephosphatase, or if the activity of the polypeptide B1897, preferably represented by SEQ ID NO. 79057, or a homolog or fragment thereof, or if the activity of a polypeptide encoded by a nucleic acid molecule comprising the nucleic acid SEQ ID NO. 79056, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 79056 or polypeptide SEQ ID NO. 79057, respectively, or a homolog or a fragment thereof, is increased or generated, or if the activity trehalose-phosphatase is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 36 to 153-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a b1921-protein, or if the activity of the polypeptide B1921, preferably represented by SEQ ID NO. 112170, or a homolog or fragment thereof, or if the activity of a polypeptide encoded by a nucleic acid molecule comprising the nucleic acid SEQ ID NO. 112169, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 112169 or polypeptide SEQ ID NO. 112170, respectively, or a homolog or a fragment thereof, is increased or generated, or if the activity b1921-protein is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 46 to 84-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a O-antigen chain length determinant, or if the activity of the polypeptide B2027, preferably represented by SEQ ID NO. 79218, or a homolog or fragment thereof, or if the activity of a polypeptide encoded by a nucleic acid molecule comprising the nucleic acid SEQ ID NO. 79217, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 79217 or polypeptide SEQ ID NO. 79218, respectively, or a homolog or a fragment thereof, is increased or generated, or if the activity O-antigen chain length determinant is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 39 to 203-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a colanic acid biosynthesis protein, or if the activity of the polypeptide B2043, preferably represented by SEQ ID NO. 112194, or a homolog or fragment thereof, or if the activity of a polypeptide encoded by a nucleic acid molecule comprising the nucleic acid SEQ ID NO. 112193, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 112193 or polypeptide SEQ ID NO. 112194, respectively, or a homolog or a fragment thereof, is increased or generated, or if the activity colanic acid biosynthesis protein is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 38 to 60-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a b2360-protein, or if the activity of the polypeptide B2360, preferably represented by SEQ ID NO. 39220, or a homolog or fragment thereof, or if the activity of a polypeptide encoded by a nucleic acid molecule comprising the nucleic acid SEQ ID NO. 39219, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 39219 or polypeptide SEQ ID NO. 39220, respectively, or a homolog or a fragment thereof, is increased or generated, or if the activity b2360-protein is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 48 to 93-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a putative transport system permease protein, or if the activity of the polypeptide B2546, preferably represented by SEQ ID NO. 99899, or a homolog or fragment thereof, or if the activity of a polypeptide encoded by a nucleic acid molecule comprising the nucleic acid SEQ ID NO. 99898, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 99898 or polypeptide SEQ ID NO. 99899, respectively, or a homolog or a fragment thereof, is increased or generated, or if the activity putative transport system permease protein is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 53 to 87-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a b2548-protein, or if the activity of the polypeptide B2548, preferably represented by SEQ ID NO. 40638, or a homolog or fragment thereof, or if the activity of a polypeptide encoded by a nucleic acid molecule comprising the nucleic acid SEQ ID NO. 40637, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 40637 or polypeptide SEQ ID NO. 40638, respectively, or a homolog or a fragment thereof, is increased or generated, or if the activity b2548-protein is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 35 to 100-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a b2613-protein, or if the activity of the polypeptide B2613, preferably represented by SEQ ID NO. 40666, or a homolog or fragment thereof, or if the activity of a polypeptide encoded by a nucleic acid molecule comprising the nucleic acid SEQ ID NO. 40665, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 40665 or polypeptide SEQ ID NO. 40666, respectively, or a homolog or a fragment thereof, is increased or generated, or if the activity b2613-protein is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 39 to 100-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a b2673-protein, or if the activity of the polypeptide B2673, preferably represented by SEQ ID NO. 9245, or a homolog or fragment thereof, or if the activity of a polypeptide encoded by a nucleic acid molecule comprising the nucleic acid SEQ ID NO. 9244, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 9244 or polypeptide SEQ ID NO. 9245, respectively, or a homolog or a fragment thereof, is increased or generated, or if the activity b2673-protein is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 36 to 55-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a murein transglycosylase, or if the activity of the polypeptide B2701, preferably represented by SEQ ID NO. 80757, or a homolog or fragment thereof, or if the activity of a polypeptide encoded by a nucleic acid molecule comprising the nucleic acid SEQ ID NO. 80756, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 80756 or polypeptide SEQ ID NO. 80757, respectively, or a homolog or a fragment thereof, is increased or generated, or if the activity murein transglycosylase is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 49 to 174-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a b2812-protein, or if the activity of the polypeptide B2812, preferably represented by SEQ ID NO. 40796, or a homolog or fragment thereof, or if the activity of a polypeptide encoded by a nucleic acid molecule comprising the nucleic acid SEQ ID NO. 40795, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 40795 or polypeptide SEQ ID NO. 40796, respectively, or a homolog or a fragment thereof, is increased or generated, or if the activity b2812-protein is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 34 to 83-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a amino-acid acetyltransferase, or if the activity of the polypeptide B2818, preferably represented by SEQ ID NO. 81577, or a homolog or fragment thereof, or if the activity of a polypeptide encoded by a nucleic acid molecule comprising the nucleic acid SEQ ID NO. 81576, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 81576 or polypeptide SEQ ID NO. 81577, respectively, or a homolog or a fragment thereof, is increased or generated, or if the activity amino-acid acetyltransferase is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 36 to 39-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a aminoacid acetyltransferase, or if the activity of the polypeptide B2818, preferably represented by SEQ ID NO. 81577, or a homolog or fragment thereof, or if the activity of a polypeptide encoded by a nucleic acid molecule comprising the nucleic acid SEQ ID NO. 81576, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 81576 or polypeptide SEQ ID NO. 81577, respectively, or a homolog or a fragment thereof, is increased or generated, or if the activity amino-acid acetyltransferase is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 33 to 153-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a glycolate utilization protein, or if the activity of the polypeptide B2977, preferably represented by SEQ ID NO. 112217, or a homolog or fragment thereof, or if the activity of a polypeptide encoded by a nucleic acid molecule comprising the nucleic acid SEQ ID NO. 112216, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 112216 or polypeptide SEQ ID NO. 112217, respectively, or a homolog or a fragment thereof, is increased or generated, or if the activity glycolate utilization protein is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 42 to 53-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a ABC transporter permease subunit, or if the activity of the polypeptide B3194, preferably represented by SEQ ID NO. 112381, or a homolog or fragment thereof, or if the activity of a polypeptide encoded by a nucleic acid molecule comprising the nucleic acid SEQ ID NO. 112380, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 112380 or polypeptide SEQ ID NO. 112381, respectively, or a homolog or a fragment thereof, is increased or generated, or if the activity ABC transporter permease subunit is increased or generated in a non-human organism, like a microorganism or a plant cell, plant or part thereof, especially with plastidic localization, whereby the respective line disclose in column 7 the fine chemical alpha-tocopherol. For example, an increase of the alpha-tocopherol of at least 1 percent, particularly in a range of 25 to 26-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a lipopolysaccharide biosynthesis protein, or if the activity of the polypeptide B3790, preferably represented by SEQ ID NO. 112607, or a homolog or fragment thereof, or if the activity of a polypeptide encoded by a nucleic acid molecule comprising the nucleic acid SEQ ID NO. 112606, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 112606 or polypeptide SEQ ID NO. 112607, respectively, or a homolog or a fragment thereof, is increased or generated, or if the activity lipopolysaccharide biosynthesis protein is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1-percent, particularly in a range of 35 to 61-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a b3890-protein, or if the activity of the polypeptide B3890, preferably represented by SEQ ID NO. 112635, or a homolog or fragment thereof, or if the activity of a polypeptide encoded by a nucleic acid molecule comprising the nucleic acid SEQ ID NO. 112634, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 112634 or polypeptide SEQ ID NO. 112635, respectively, or a homolog or a fragment thereof, is increased or generated, or if the activity b3890-protein is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 67 to 290-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a glycerol dehydrogenase, or if the activity of the polypeptide B3945, preferably represented by SEQ ID NO. 44224, or a homolog or fragment thereof, or if the activity of a polypeptide encoded by a nucleic acid molecule comprising the nucleic acid SEQ ID NO. 44223, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 44223 or polypeptide SEQ ID NO. 44224, respectively, or a homolog or a fragment thereof, is increased or generated, or if the activity glycerol dehydrogenase is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 31 to 56-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a b3989-protein, or if the activity of the polypeptide B3989, preferably represented by SEQ ID NO. 44373, or a homolog or fragment thereof, or if the activity of a polypeptide encoded by a nucleic acid molecule comprising the nucleic acid SEQ ID NO. 44372, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 44372 or polypeptide SEQ ID NO. 44373, respectively, or a homolog or a fragment thereof, is increased or generated, or if the activity b3989-protein is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 55 to 120-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a b3989-protein, or if the activity of the polypeptide B3989, preferably represented by SEQ ID NO. 44373, or a homolog or fragment thereof, or if the activity of a polypeptide encoded by a nucleic acid molecule comprising the nucleic acid SEQ ID NO. 44372, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 44372 or polypeptide SEQ ID NO. 44373, respectively, or a homolog or a fragment thereof, is increased or generated, or if the activity b3989-protein is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 35 to 102-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a b4029-protein, or if the activity of the polypeptide B4029, preferably represented by SEQ ID NO. 10741, or a homolog or fragment thereof, or if the activity of a polypeptide encoded by a nucleic acid molecule comprising the nucleic acid SEQ ID NO. 10740, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 10740 or polypeptide SEQ ID NO. 10741, respectively, or a homolog or a fragment thereof, is increased or generated, or if the activity b4029-protein is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 38 to 244-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a b4050-protein, or if the activity of the polypeptide B4050, preferably represented by SEQ ID NO. 106533, or a homolog or fragment thereof, or if the activity of a polypeptide encoded by a nucleic acid molecule comprising the nucleic acid SEQ ID NO. 106532, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 106532 or polypeptide SEQ ID NO. 106533, respectively, or a homolog or a fragment thereof, is increased or generated, or if the activity b4050-protein is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 27 to 39-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a b4056-protein, or if the activity of the polypeptide B4056, preferably represented by SEQ ID NO. 94543, or a homolog or fragment thereof, or if the activity of a polypeptide encoded by a nucleic acid molecule comprising the nucleic acid SEQ ID NO. 94542, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 94542 or polypeptide SEQ ID NO. 94543, respectively, or a homolog or a fragment thereof, is increased or generated, or if the activity b4056-protein is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 41 to 55-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a acetyltransferase, or if the activity of the polypeptide B4256, preferably represented by SEQ ID NO. 45322, or a homolog or fragment thereof, or if the activity of a polypeptide encoded by a nucleic acid molecule comprising the nucleic acid SEQ ID NO. 45321, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 45321 or polypeptide SEQ ID NO. 45322, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 69 to 93-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a acetyltransferase, or if the activity of the polypeptide B4256, preferably represented by SEQ ID NO. 45322, or a homolog or fragment thereof, or if the activity of a polypeptide encoded by a nucleic acid molecule comprising the nucleic acid SEQ ID NO. 45321, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 45321 or polypeptide SEQ ID NO. 45322, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 61 to 132-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a gluconate transport system permease 3, or if the activity of the polypeptide B4321, preferably represented by SEQ ID NO. 45395, or a homolog or fragment thereof, or if the activity of a polypeptide encoded by a nucleic acid molecule comprising the nucleic acid SEQ ID NO. 45394, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 45394 or polypeptide SEQ ID NO. 45395, respectively, or a homolog or a fragment thereof, is increased or generated, or if the activity gluconate transport system permease 3 is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 39 to 72-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a gluconate transport system permease 3, or if the activity of the polypeptide B4321, preferably represented by SEQ ID NO. 45395, or a homolog or fragment thereof, or if the activity of a polypeptide encoded by a nucleic acid molecule comprising the nucleic acid SEQ ID NO. 45394, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 45394 or polypeptide SEQ ID NO. 45395, respectively, or a homolog or a fragment thereof, is increased or generated, or if the activity gluconate transport system permease 3 is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 97 to 248-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a thioredoxin, or if the activity of the polypeptide C_pp004096192r, preferably represented by SEQ ID NO. 45758, or a homolog or fragment thereof, or if the activity of a polypeptide encoded by a nucleic acid molecule comprising the nucleic acid SEQ ID NO. 45757, preferably the coding region thereof, or a homolog or fragment thereof, e.g. derived from Physcomitrella patens, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 45757 or polypeptide SEQ ID NO. 45758, respectively, or a homolog or a fragment thereof, is increased or generated, or if the activity thioredoxin is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 35 to 147-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a glutaredoxin, or if the activity of the polypeptide GM02LC12622, preferably represented by SEQ ID NO. 10812, or a homolog or fragment thereof, or if the activity of a polypeptide encoded by a nucleic acid molecule comprising the nucleic acid SEQ ID NO. 10811, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 10811 or polypeptide SEQ ID NO. 10812, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 57 to 105-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a glutaredoxin, or if the activity of the polypeptide GM02LC12622, preferably represented by SEQ ID NO. 10812, or a homolog or fragment thereof, or if the activity of a polypeptide encoded by a nucleic acid molecule comprising the nucleic acid SEQ ID NO. 10811, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 10811 or polypeptide SEQ ID NO. 10812, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 34 to 204-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a transcription factor, or if the activity of the polypeptide GM02LC21368, preferably represented by SEQ ID NO. 84199, or a homolog or fragment thereof, or if the activity of a polypeptide encoded by a nucleic acid molecule comprising the nucleic acid SEQ ID NO. 84198, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 84198 or polypeptide SEQ ID NO. 84199, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 48 to 185-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a polyphosphate kinase, or if the activity of the polypeptide Sll0290, preferably represented by SEQ ID NO. 49829, or a homolog or fragment thereof, or if the activity of a polypeptide encoded by a nucleic acid molecule comprising the nucleic acid SEQ ID NO. 49828, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 49828 or polypeptide SEQ ID NO. 49829, respectively, or a homolog or a fragment thereof, is increased or generated, or if the activity polyphosphate kinase is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 27 to 49-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a phosphofructokinase, or if the activity of the polypeptide Sll0745, preferably represented by SEQ ID NO. 112662, or a homolog or fragment thereof, or if the activity of a polypeptide encoded by a nucleic acid molecule comprising the nucleic acid SEQ ID NO. 112661, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 112661 or polypeptide SEQ ID NO. 112662, respectively, or a homolog or a fragment thereof, is increased or generated, or if the activity phosphofructokinase is increased or generated in a non-human organism, like a microorganism or a plant cell, plant or part thereof, especially with mitochondrial localization, whereby the respective line disclose in column 7 the fine chemical gamma-tocopherol. For example, an increase of the gamma-tocopherol of at least 1 percent, particularly in a range of 33 to 76-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a coproporphyrinogen oxidase, or if the activity of the polypeptide Sll1185, preferably represented by SEQ ID NO. 53190, or a homolog or fragment thereof, or if the activity of a polypeptide encoded by a nucleic acid molecule comprising the nucleic acid SEQ ID NO. 53189, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 53189 or polypeptide SEQ ID NO. 53190, respectively, or a homolog or a fragment thereof, is increased or generated, or if the activity coproporphyrinogen oxidase is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 28 to 42-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a sll1761-protein, or if the activity of the polypeptide Sll1761, preferably represented by SEQ ID NO. 55380, or a homolog or fragment thereof, or if the activity of a polypeptide encoded by a nucleic acid molecule comprising the nucleic acid SEQ ID NO. 55379, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 55379 or polypeptide SEQ ID NO. 55380, respectively, or a homolog or a fragment thereof, is increased or generated, or if the activity sll1761-protein is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 33 to 68-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a adenylate kinase, or if the activity of the polypeptide Sll1815, preferably represented by SEQ ID NO. 85809, or a homolog or fragment thereof, or if the activity of a polypeptide encoded by a nucleic acid molecule comprising the nucleic acid SEQ ID NO. 85808, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 85808 or polypeptide SEQ ID NO. 85809, respectively, or a homolog or a fragment thereof, is increased or generated, or if the activity adenylate kinase is increased or generated in a non-human organism, like a microorganism or a plant cell, plant or part thereof, especially with mitochondrial localization, whereby the respective line disclose in column 7 the fine chemical gamma-tocopherol. For example, an increase of the gamma-tocopherol of at least 1 percent, particularly in a range of 32 to 45-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a precorrin methylase, or if the activity of the polypeptide Slr0239, preferably represented by SEQ ID NO. 86541, or a homolog or fragment thereof, or if the activity of a polypeptide encoded by a nucleic acid molecule comprising the nucleic acid SEQ ID NO. 86540, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 86540 or polypeptide SEQ ID NO. 86541, respectively, or a homolog or a fragment thereof, is increased or generated, or if the activity precorrin methylase is increased or generated in a non-human organism, like a microorganism or a plant cell, plant or part thereof, especially with mitochondrial localization, whereby the respective line disclose in column 7 the fine chemical alpha-tocopherol. For example, an increase of the alpha-tocopherol of at least 1 percent, particularly in a range of 39 to 46-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a malate dehydrogenase, or if the activity of the polypeptide Slr0721, preferably represented by SEQ ID NO. 87398, or a homolog or fragment thereof, or if the activity of a polypeptide encoded by a nucleic acid molecule comprising the nucleic acid SEQ ID NO. 87397, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 87397 or polypeptide SEQ ID NO. 87398, respectively, or a homolog or a fragment thereof, is increased or generated, or if the activity malate dehydrogenase is increased or generated in a non-human organism, like a microorganism or a plant cell, plant or part thereof, especially with plastidic localization, whereby the respective line disclose in column 7 the fine chemical alpha-tocopherol. For example, an increase of the alpha-tocopherol of at least 1 percent, particularly in a range of 60 to 125-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a malate dehydrogenase, or if the activity of the polypeptide Slr0721, preferably represented by SEQ ID NO. 87398, or a homolog or fragment thereof, or if the activity of a polypeptide encoded by a nucleic acid molecule comprising the nucleic acid SEQ ID NO. 87397, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 87397 or polypeptide SEQ ID NO. 87398, respectively, or a homolog or a fragment thereof, is increased or generated, or if the activity malate dehydrogenase is increased or generated in a non-human organism, like a microorganism or a plant cell, plant or part thereof, especially with plastidic localization, whereby the respective line disclose in column 7 the fine chemical gamma-tocopherol. For example, an increase of the gamma-tocopherol of at least 1 percent, particularly in a range of 43 to 268-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a geranylgeranyl pyrophosphate synthase, or if the activity of the polypeptide Slr0739, preferably represented by SEQ ID NO. 57236, or a homolog or fragment thereof, or if the activity of a polypeptide encoded by a nucleic acid molecule comprising the nucleic acid SEQ ID NO. 57235, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 57235 or polypeptide SEQ ID NO. 57236, respectively, or a homolog or a fragment thereof, is increased or generated, or if the activity geranylgeranyl pyrophosphate synthase is increased or generated in a non-human organism, like a microorganism or a plant cell, plant or part thereof, especially with plastidic localization, whereby the respective line disclose in column 7 the fine chemical alpha-tocopherol. For example, an increase of the alpha-tocopherol of at least 1 percent, partitularly in a range of 45 to 82-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a geranylgeranyl pyrophosphate synthase, or if the activity of the polypeptide Slr0739, preferably represented by SEQ ID NO. 57236, or a homolog or fragment thereof, or if the activity of a polypeptide encoded by a nucleic acid molecule comprising the nucleic acid SEQ ID NO. 57235, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 57235 or polypeptide SEQ ID NO. 57236, respectively, or a homolog or a fragment thereof, is increased or generated, or if the activity geranylgeranyl pyrophosphate synthase is increased or generated in a non-human organism, like a microorganism or a plant cell, plant or part thereof, especially with plastidic localization, whereby the respective line disclose in column 7 the fine chemical gamma-tocopherol. For example, an increase of the gamma-tocopherol of at least 1 percent, particularly in a range of 91 to 144-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a carbohydrate kinase, or if the activity of the polypeptide Slr1420, preferably represented by SEQ ID NO. 58591, or a homolog or fragment thereof, or if the activity of a polypeptide encoded by a nucleic acid molecule comprising the nucleic acid SEQ ID NO. 58590, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 58590 or polypeptide SEQ ID NO. 58591, respectively, or a homolog or a fragment thereof, is increased or generated, or if the activity carbohydrate kinase is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 33 to 50-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a photosystem II protein, or if the activity of the polypeptide Slr1739, preferably represented by SEQ ID NO. 58732, or a homolog or fragment thereof, or if the activity of a polypeptide encoded by a nucleic acid molecule comprising the nucleic acid SEQ ID NO. 58731, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 58731 or polypeptide SEQ ID NO. 58732, respectively, or a homolog or a fragment thereof, is increased or generated, or if the activity photosystem II protein is increased or generated in a non-human organism, like a microorganism or a plant cell, plant or part thereof, especially with plastidic localization, whereby the respective line disclose in column 7 the fine chemical gamma-tocopherol. For example, an increase of the gamma-tocopherol of at least 1 percent, particularly in a range of 77 to 203-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a glycerol-3-phosphate dehydrogenase, or if the activity of the polypeptide Slr1755, preferably represented by SEQ ID NO. 58824, or a homolog or fragment thereof, or if the activity of a polypeptide encoded by a nucleic acid molecule comprising the nucleic acid SEQ ID NO. 58823, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 58823 or polypeptide SEQ ID NO. 58824, respectively, or a homolog or a fragment thereof, is increased or generated, or if the activity glycerol-3-phosphate dehydrogenase is increased or generated in a non-human organism, like a microorganism or a plant cell, plant or part thereof, especially with plastidic localization, whereby the respective line disclose in column 7 the fine chemical alpha-tocopherol. For example, an increase of the alpha-tocopherol of at least 1 percent, particularly in a range of 37 to 81-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a phosphoadenosine phosphosulfate reductase , or if the activity of the polypeptide Slr1791, preferably represented by SEQ ID NO. 12141, or a homolog or fragment thereof, or if the activity of a polypeptide encoded by a nucleic acid molecule comprising the nucleic acid SEQ ID NO. 12140, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 12140 or polypeptide SEQ ID NO. 12141, respectively, or a homolog or a fragment thereof, is increased or generated, or if the activity phosphoadenosine phosphosulfate reductase is increased or generated in a non-human organism, like a microorganism or a plant cell, plant or part thereof, especially with plastidic localization, whereby the respective line disclose in column 7 the fine chemical gamma-tocopherol. For example, an increase of the gamma-tocopherol of at least 1 percent, particularly in a range of 33 to 58-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a slr1917-protein, or if the activity of the polypeptide Slr1917, preferably represented by SEQ ID NO. 113039, or a homolog or fragment thereof, or if the activity of a polypeptide encoded by a nucleic acid molecule comprising the nucleic acid SEQ ID NO. 113038, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 113038 or polypeptide SEQ ID NO. 113039, respectively, or a homolog or a fragment thereof, is increased or generated, or if the activity slr1917-protein is increased or generated in a non-human organism, like a microorganism or a plant cell, plant or part thereof, especially with plastidic localization, whereby the respective line disclose in column 7 the fine chemical alpha-tocopherol. For example, an increase of the alpha-tocopherol of at least 1 percent, particularly in a range of 36 to 70-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a slr1917-protein, or if the activity of the polypeptide Slr1917, preferably represented by SEQ ID NO. 113039, or a homolog or fragment thereof, or if the activity of a polypeptide encoded by a nucleic acid molecule comprising the nucleic acid SEQ ID NO. 113038, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 113038 or polypeptide SEQ ID NO. 113039, respectively, or a homolog or a fragment thereof, is increased or generated, or if the activity slr1917-protein is increased or generated in a non-human organism, like a microorganism or a plant cell, plant or part thereof, especially with plastidic localization, whereby the respective line disclose in column 7 the fine chemical gamma-tocopherol. For example, an increase of the gamma-tocopherol of at least 1 percent, particularly in a range of 144 to 540-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a Sec-independent protein translocase subunit, or if the activity of the polypeptide TTC0019, preferably represented by SEQ ID NO. 12699, or a homolog or fragment thereof, or if the activity of a polypeptide encoded by a nucleic acid molecule comprising the nucleic acid SEQ ID NO. 12698, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 12698 or polypeptide SEQ ID NO. 12699, respectively, or a homolog or a fragment thereof, is increased or generated, or if the activity Sec-independent protein translocase subunit is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 53 to 129-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a Sec-independent protein translocase subunit, or if the activity of the polypeptide TTC0019, preferably represented by SEQ ID NO. 12699, or a homolog or fragment thereof, or if the activity of a polypeptide encoded by a nucleic acid molecule comprising the nucleic acid SEQ ID NO. 12698, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 12698 or polypeptide SEQ ID NO. 12699, respectively, or a homolog or a fragment thereof, is increased or generated, or if the activity Sec-independent protein translocase subunit is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 35 to 264-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a multiple antibiotic resistance protein, or if the activity of the polypeptide TTC1193, preferably represented by SEQ ID NO. 61724, or a homolog or fragment thereof, or if the activity of a polypeptide encoded by a nucleic acid molecule comprising the nucleic acid SEQ ID NO. 61723, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 61723 or polypeptide SEQ ID NO. 61724, respectively, or a homolog or a fragment thereof, is increased or generated, or if the activity multiple antibiotic resistance protein is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 88 to 166-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a TTC1386-protein, or if the activity of the polypeptide TTC1386, preferably represented by SEQ ID NO. 62080, or a homolog or fragment thereof, or if the activity of a polypeptide encoded by a nucleic acid molecule comprising the nucleic acid SEQ ID NO. 62079, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 62079 or polypeptide SEQ ID NO. 62080, respectively, or a homolog or a fragment thereof, is increased or generated, or if the activity TTC1386-protein is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 52 to 132-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a TTC1386-protein, or if the activity of the polypeptide TTC1386, preferably represented by SEQ ID NO. 62080, or a homolog or fragment thereof, or if the activity of a polypeptide encoded by a nucleic acid molecule comprising the nucleic acid SEQ ID NO. 62079, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 62079 or polypeptide SEQ ID NO. 62080, respectively, or a homolog or a fragment thereof, is increased or generated, or if the activity TTC1386-protein is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 38 to 171-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a homocitrate synthase, or if the activity of the polypeptide TTC1550, preferably represented by SEQ ID NO. 12975, or a homolog or fragment thereof, or if the activity of a polypeptide encoded by a nucleic acid molecule comprising the nucleic acid SEQ ID NO. 12974, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 12974 or polypeptide SEQ ID NO. 12975, respectively, or a homolog or a fragment thereof, is increased or generated, or if the activity homocitrate synthase is increased or generated in a non-human organism, like a microorganism or a plant cell, plant or part thereof, especially with non-targeted localization, whereby the respective line disclose in column 7 the fine chemical alpha-tocopherol. For example, an increase of the alpha-tocopherol of at least 1 percent, particularly in a range of 86 to 106-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a 3-deoxy-7-phosphoheptulonate synthase, or if the activity of the polypeptide Ybr249c, preferably represented by SEQ ID NO. 88880, or a homolog or fragment thereof, or if the activity of a polypeptide encoded by a nucleic acid molecule comprising the nucleic acid SEQ ID NO. 88879, preferably the coding region thereof, or a homolog or fragment thereof, e.g. derived from Sac-charomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 88879 or polypeptide SEQ ID NO. 88880, respectively, or a homolog or a fragment thereof, is increased or generated, or if the activity 3-deoxy-7-phosphoheptulonate synthase is increased or generated in a non-human organism, like a microorganism or a plant cell, plant or part thereof, especially with plastidic localization, whereby the respective line disclose in column 7 the fine chemical alpha-tocopherol. For example, an increase of the alpha-tocopherol of at least 1 percent, particularly in a range of 76 to 142-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a 3-deoxy-7-phosphoheptulonate synthase, or if the activity of the polypeptide Ybr249c, preferably represented by SEQ ID NO. 88880, or a homolog or fragment thereof, or if the activity of a polypeptide encoded by a nucleic acid molecule comprising the nucleic acid SEQ ID NO. 88879, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 88879 or polypeptide SEQ ID NO. 88880, respectively, or a homolog or a fragment thereof, is increased or generated, or if the activity 3-deoxy-7-phosphoheptulonate synthase is increased or generated in a non-human organism, like a microorganism or a plant cell, plant or part thereof, especially with plastidic localization, whereby the respective line disclose in column 7 the fine chemical gamma-tocopherol. For example, an increase of the gamma-tocopherol of at least 1 percent, particularly in a range of 51 to 168-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a ycl049c-protein, or if the activity of the polypeptide Yc1049c, preferably represented by SEQ ID NO. 113221, or a homolog or fragment thereof, or if the activity of a polypeptide encoded by a nucleic acid molecule comprising the nucleic acid SEQ ID NO. 113220, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 113220 or polypeptide SEQ ID NO. 113221, respectively, or a homolog or a fragment thereof, is increased or generated, or if the activity ycl049c-protein is increased or generated in a non-human organism, like a microorganism or a plant cell, plant or part thereof, especially with plastidic localization, whereby the respective line disclose in column 7 the fine chemical gamma-tocopherol. For example, an increase of the gamma-tocopherol of at least 1 percent, particularly in a range of 57 to 92-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a homocitrate synthase, or if the activity of the polypeptide Ydl131w, preferably represented by SEQ ID NO. 113225, or a homolog or fragment thereof, or if the activity of a polypeptide encoded by a nucleic acid molecule comprising the nucleic acid SEQ ID NO. 113224, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 113224 or polypeptide SEQ ID NO. 113225, respectively, or a homolog or a fragment thereof, is increased or generated, or if the activity homocitrate synthase is increased or generated in a non-human organism, like a microorganism or a plant cell, plant or part thereof, especially with plastidic localization, whereby the respective line disclose in column 7 the fine chemical alpha-tocopherol. For example, an increase of the alpha-tocopherol of at least 1 percent, particularly in a range of 66 to 285-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a ydl235c-protein, or if the activity of the polypeptide Ydl235c, preferably represented by SEQ ID NO. 113314, or a homolog or fragment thereof, or if the activity of a polypeptide encoded by a nucleic acid molecule comprising the nucleic acid SEQ ID NO. 113313, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 113313 or polypeptide SEQ ID NO. 113314, respectively, or a homolog or a fragment thereof, is increased or generated, or if the activity ydl235c-protein is increased or generated in a non-human organism, like a microorganism or a plant cell, plant or part thereof, especially with plastidic localization, whereby the respective line disclose in column 7 the fine chemical alpha-tocopherol. For example, an increase of the alpha-tocopherol of at least 1 percent, particularly in a range of 55 to 199-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a N-(5′-phosphoribosyl)anthranilate isomerase, or if the activity of the polypeptide Ydr007w, preferably represented by SEQ ID NO. 89974, or a homolog or fragment thereof, or if the activity of a polypeptide encoded by a nucleic acid molecule comprising the nucleic acid SEQ ID NO. 89973, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 89973 or polypeptide SEQ ID NO. 89974, respectively, or a homolog or a fragment thereof, is increased or generated, or if the activity N-(5′-phosphoribosyl)anthranilate isomerase is increased or generated in a non-human organism, like a microorganism or a plant cell, plant or part thereof, especially with plastidic localization, whereby the respective line disclose in column 7 the fine chemical gamma-tocopherol. For example, an increase of the gamma-tocopherol of at least 1 percent, particularly in a range of 70 to 140-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a phospho 2-dehydro-3-deoxyheptonate aldolase, or if the activity of the polypeptide Ydr035w, preferably represented by SEQ ID NO. 113331, or a homolog or fragment thereof, or if the activity of a polypeptide encoded by a nucleic acid molecule comprising the nucleic acid SEQ ID NO. 113330, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 113330 or polypeptide SEQ ID NO. 113331, respectively, or a homolog or a fragment thereof, is increased or generated, or if the activity phospho-2-dehydro-3-deoxyheptonate aldolase is increased or generated in a non-human organism, like a microorganism or a plant cell, plant or part thereof, especially with plastidic localization, whereby the respective line disclose in column 7 the fine chemical gamma-tocopherol. For example, an increase of the gamma-tocopherol of at least 1 percent, particularly in a range of 39 to 106-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a ydr183w-protein, or if the activity of the polypeptide Ydr183w, preferably represented by SEQ ID NO. 90104, or a homolog or fragment thereof, or if the activity of a polypeptide encoded by a nucleic acid molecule comprising the nucleic acid SEQ ID NO. 90103, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 90103 or polypeptide SEQ ID NO. 90104, respectively, or a homolog or a fragment thereof, is increased or generated, or if the activity ydr183w-protein is increased or generated in a non-human organism, like a microorganism or a plant cell, plant or part thereof, especially with plastidic localization, whereby the respective line disclose in column 7 the fine chemical gamma-tocopherol. For example, an increase of the gamma-tocopherol of at least 1 percent, particularly in a range of 35 to 105-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a yer106w-protein, or if the activity of the polypeptide Yer106w, preferably represented by SEQ ID NO. 63804, or a homolog or fragment thereof, or if the activity of a polypeptide encoded by a nucleic acid molecule comprising the nucleic acid SEQ ID NO. 63803, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 63803 or polypeptide SEQ ID NO. 63804, respectively, or a homolog or a fragment thereof, is increased or generated, or if the activity yer106w-protein is increased or generated in a non-human organism, like a microorganism or a plant cell, plant or part thereof, especially with plastidic localization, whereby the respective line disclose in column 7 the fine chemical gamma-tocopherol. For example, an increase of the gamma-tocopherol of at least 1 percent, particularly in a range of 34 to 302-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a small nuclear ribonucleoprotein , or if the activity of the polypeptide Yer112w, preferably represented by SEQ ID NO. 113599, or a homolog or fragment thereof, or if the activity of a polypeptide encoded by a nucleic acid molecule comprising the nucleic acid SEQ ID NO. 113598, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 113598 or polypeptide SEQ ID NO. 113599, respectively, or a homolog or a fragment thereof, is increased or generated, or if the activity small nuclear ribonucleoprotein is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 51 to 252-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a phosphatidylserine decarboxylase, or if the activity of the polypeptide Ygr170w, preferably represented by SEQ ID NO. 113637, or a homolog or fragment thereof, or if the activity of a polypeptide encoded by a nucleic acid molecule comprising the nucleic acid SEQ ID NO. 113636, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 113636 or polypeptide SEQ ID NO. 113637, respectively, or a homolog or a fragment thereof, is increased or generated, or if the activity phosphatidylserine decarboxylase is increased or generated in a non-human organism, like a microorganism or a plant cell, plant or part thereof, especially with plastidic localization, whereby the respective line disclose in column 7 the fine chemical alpha-tocopherol. For example, an increase of the alpha-tocopherol of at least 1 percent, particularly in a range of 42 to 62-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a polygalacturonase, or if the activity of the polypeptide Yjr153w, preferably represented by SEQ ID NO. 66275, or a homolog or fragment thereof, or if the activity of a polypeptide encoded by a nucleic acid molecule comprising the nucleic acid SEQ ID NO. 66274, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 66274 or polypeptide SEQ ID NO. 66275, respectively, or a homolog or a fragment thereof, is increased or generated, or if the activity polygalacturonase is increased or generated in a non-human organism, like a microorganism or a plant cell, plant or part thereof, especially with non-targeted localization, whereby the respective line disclose in column 7 the fine chemical gamma-tocopherol. For example, an increase of the gamma-tocopherol of at least 1 percent, particularly in a range of 89 to 115-percent is conferred as compared to a corresponding non-transformed wild type non-human organism.

Accordingly, in one embodiment, an increase of gamma-tocopherol in a non-human organism, as compared to a corresponding non-transformed wild type non-human organism, is conferred in the process of the invention, if the activity of a polypeptide showing the activity of a ylr178c-protein, or if the activity of the polypeptide Ylr178c, preferably represented by SEQ ID NO. 66716, or a homolog or fragment thereof, or if the activity of a polypeptide encoded by a nucleic acid molecule comprising the nucleic acid SEQ ID NO. 66715, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 66715 or polypeptide SEQ ID NO. 66716, respectively, or a homolog or a fragment thereof, is increased or generated, or if the activity ylr178c-protein is increased or generated in a non-human organism, like a microorganism or a plant cell, plant or part thereof, especially with plastidic localization, whereby the respective line disclose in column 7 the fine chemical gamma-tocopherol. For example, an increase of the gamma-tocopherol of at least 1 percent, particularly in a range of 39 to 62-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a adenylosuccinate lyase, or if the activity of the polypeptide Ylr359w, preferably represented by SEQ ID NO. 67191, or a homolog or fragment thereof, or if the activity of a polypeptide encoded by a nucleic acid molecule comprising the nucleic acid SEQ ID NO. 67190, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 67190 or polypeptide SEQ ID NO. 67191, respectively, or a homolog or a fragment thereof, is increased or generated, or if the activity adenylosuccinate lyase is increased or generated in a non-human organism, like a microorganism or a plant cell, plant or part thereof, especially with plastidic localization, whereby the respective line disclose in column 7 the fine chemical alpha-tocopherol. For example, an increase of the alpha-tocopherol of at least 1 percent, particularly in a range of 54 to 78-percent is conferred as compared to a corresponding non-transformed wild type non-human organism.

Accordingly, in one embodiment, an increase of alpha-tocopherol in a non-human organism, as compared to a corresponding non-transformed wild type non-human organism, is conferred in the process of the invention, if the activity of a polypeptide showing the activity of a ypr170c-protein, or if the activity of the polypeptide Ypr170c, preferably represented by SEQ ID NO. 113648, or a homolog or fragment thereof, or if the activity of a polypeptide encoded by a nucleic acid molecule comprising the nucleic acid SEQ ID NO. 113647, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 7, column 5 or 8 in the respective same line as the nucleic acid molecule SEQ ID NO. 113647 or polypeptide SEQ ID NO. 113648, respectively, or a homolog or a fragment thereof, is increased or generated, or if the activity ypr170c-protein is increased or generated in a non-human organism, like a microorganism or a plant cell, plant or part thereof, especially with plastidic localization, whereby the respective line disclose in column 7 the fine chemical alpha-tocopherol. For example, an increase of the alpha-tocopherol of at least 1 percent, particularly in a range of 59 to 73-percent is conferred as compared to a corresponding non-transformed wild type non-human organism.

In an embodiment of the invention, a protein or polypeptide has the “activity of a protein as shown in the respective line in Table II, column 3” if its de novo activity, or its increased expression directly or indirectly leads to an increase in the fine chemical level in the organism or a part thereof, preferably in a cell of said organism, and the protein has the above-mentioned activities of a protein as shown in the respective line in Table II, column 3. Throughout the specification the activity or preferably the biological activity of such a protein or polypeptide or an nucleic acid molecule or sequence encoding such protein or polypeptide is identical or similar if it still has the biological or enzymatic activity of a protein as shown in the respective line in Table II, column 3, or which has at least 10% of the original enzymatic activity, preferably at least 20%, 30%, 40%, 50%, 60%, 70%, 80%, 90%, 95% or 99% in comparison to a protein as shown in Table II, column 3.

A series of mechanisms exists via which a modification of a protein, e.g. the polypeptide of the invention can directly or indirectly affect the yield, production and/or production efficiency of the fine chemical.

For example, the molecule number or the specific activity of the polypeptide or the nucleic acid molecule may be increased. Larger amounts of the fine chemical can be produced if the polypeptide or the nucleic acid of the invention is expressed de novo in an organism lacking the activity of said protein, preferably the nucleic acid molecules as mentioned in the respective line in Table I, columns 5 or 8, preferably the coding region thereof, or a homolog or a fragment thereof, alone or, if desired in combination with a transit peptide for example as mentioned in Table a or b, respectively, or in another embodiment by introducing said nucleic acid molecules into an organelle, such as a plastid, in the transgenic organism. However, it is also possible to modify the expression of the gene which is naturally present in the organisms, for example by integrating a nucleic acid sequence, encoding an organelle targeting sequence in front (5 prime) of the coding sequence, leading to a functional preprotein, which is directed for example to the organelle.

This also applies analogously to the combined increased expression of the nucleic acid molecule of the present invention or its gene product with that of further enzymes or regulators of the fine chemical biosynthesis pathways, e.g. which are useful for the synthesis of the fine chemicals.

The increase or modulation according to this invention can be constitutive, e.g. due to a stable permanent transgenic expression or to a stable mutation in the corresponding endogenous gene encoding the nucleic acid molecule of the invention or to a modulation of the expression or of the behaviour of a gene conferring the expression of the polypeptide of the invention, or transient, e.g. due to a transient transformation or temporary addition of a modulator such as an agonist or antagonist or inducible, e.g. after transformation with a inducible construct carrying the nucleic acid molecule of the invention under control of an inducible promoter and adding the inducer, e.g. tetracycline or as described herein below.

The increase in activity of the polypeptide amounts in a cell, an organelle, an organ or a non-human organism or a part thereof preferably to at least 1%, 5%, 10%, 20%, 50%, 70%, 80%, 90%, 100%, 150% or 200%, 250% or more in comparison to the control, reference or wild type.

In an embodiment the increase in activity of the polypeptide amounts in an organelle such as a plastid or a mitochondrion. In another embodiment the increase in activity of the polypeptide amounts in the cytoplasm. In another embodiment the increase in activity of the polypeptide amounts in the cytosol.

In the context of the application the term “cytoplasm” defines the compartment of the cell to which the protein is directed by the host cell without the addition of artificial targeting sequences, which are naturally not part of the protein.

In the context of the application the term “cytosol” defines the fluid of the cell between the plasma membrane and the organelles; that means that the organelles as well as the nucleus are not part of the cytosol.

The specific activity of a polypeptide encoded by a nucleic acid molecule of the present invention or of the polypeptide of the present invention can be tested as described in the examples. In particular, the expression of a protein in question in a cell, e.g. a plant cell or a microorganism and the detection of an increase the fine chemical level in comparison to a control is an easy test and can be performed as described in the state of the art.

In this paragraph “A” means “Goffeau et al., Science 274 (5287), 546 (1996)”, “B” means “Blattner et al., Science 277 (5331), 1453 (1997)”, “C” means “Nakamura et al., Nucleic Acids Res. 28(1), 72 (2000)”, “D” means “deposition of the sequences to the EMBL/GenBank/DDBJ databases under “Sequencing of the draft genome assembly of Azotobacter vinelandii AvOP”. in June 2005 by Copeland et al.”, “E” means “Henne et al., Nat. Biotechnol. 22 (5), 547 (2004)”, “F” means “Kaul et al., Nature 408 (6814), 796 (2000)”, and “G” means “Matsumoto et al., Nature 436 (7052), 793 (2005)”.

The nucleic acid sequence of 51340801_CANOLA from Brassica napus, e.g. as shown in the respective line in column 5 of Table I, application no. 7, is unpublished. And the activity of the gene product thereof is the activity of pyruvate kinase.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “pyruvate kinase”, especially from Brassica napus or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said 51340801_CANOLA, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said 51340801_CANOLA, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said 51340801_CANOLA, or a functional equivalent or a homolog thereof as depicted in column 8 of Table II, application no. 7, preferably a homolog or functional equivalent as depicted in column 8 of Table II B, application no. 7, and being depicted in the same respective line as said 51340801_CANOLA, and preferably the activity is increased plastidic, whereby the respective line disclose in column 7 the fine chemical alpha-tocopherol.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 15532, preferably the coding region thereof, conferred the production of or the increase in alpha-tocopherol compared with the wild type control.

The nucleic acid sequence of 51340801_CANOLA from Brassica napus, e.g. as shown in the respective line in column 5 of Table I, application no. 7, is unpublished. And the activity of the gene product thereof is the activity of pyruvate kinase.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “pyruvate kinase”, especially from Brassica napus or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said 51340801_CANOLA, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said 51340801_CANOLA, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said 51340801_CANOLA, or a functional equivalent or a homolog thereof as depicted in column 8 of Table II, application no. 7, preferably a homolog or functional equivalent as depicted in column 8 of Table II B, application no. 7, and being depicted in the same respective line as said 51340801_CANOLA, and preferably the activity is increased plastidic, whereby the respective line disclose in column 7 the fine chemical gamma-tocopherol.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 15532, preferably the coding region thereof, conferred the production of or the increase in gamma-tocopherol compared with the wild type control.

The nucleic acid sequence of 59582753_SOYBEAN from Glycine max, e.g. as shown in the respective line in column 5 of Table I, application no. 7, is unpublished. And the activity of the gene product thereof is the activity of glucose-6-phosphate 1-dehydrogenase.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “glucose-6-phosphate 1-dehydrogenase”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said 59582753_SOYBEAN, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said 59582753_SOYBEAN, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said 59582753_SOYBEAN, or a functional equivalent or a homolog thereof as depicted in column 8 of Table II, application no. 7, preferably a homolog or functional equivalent as depicted in column 8 of Table II B, application no. 7, and being depicted in the same respective line as said 59582753_SOYBEAN, and preferably the activity is increased plastidic, whereby the respective line disclose in column 7 the fine chemical alpha-tocopherol.

Accordingly, in one embodiment, the molecule which activity is to be increased in the process of the invention is the gene product with an activity as a “glucose-6-phosphate 1-dehydrogenase”, preferably it is the molecule of section (a) or (b) of this paragraph.

In particular, it was observed that in plants, especially in Arabidopsis thaliana, increasing or generating the activity of a gene product plastidic with the activity of a “glucose-6-phosphate 1-dehydrogenase”, preferably being encoded by a gene comprising the nucleic acid sequence SEQ ID NO. 16883, preferably the coding region thereof, conferred the production of or the increase in alpha-tocopherol compared with the wild type control.

The nucleic acid sequence of At1g29350 from Arabidopsis thaliana, e.g. as shown in the respective line in column 5 of Table I, application no. 7, has been published in F. And the activity of the gene product thereof is the activity of At1g29350-protein.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “At1g29350-protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said At1g29350, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said At1g29350, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said At1g29350, or a functional equivalent or a homolog thereof as depicted in column 8 of Table II, application no. 7, preferably a homolog or functional equivalent as depicted in column 8 of Table II B, application no. 7, and being depicted in the same respective line as said At1g29350, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “At1g29350-protein”, preferably it is the molecule of section (a) or (b) of this paragraph.

In particular, it was observed that in plants, especially in Arabidopsis thaliana, increasing or generating the activity of a gene product non-targeted with the activity of a “At1g29350-protein”, preferably being encoded by a gene comprising the nucleic acid sequence SEQ ID NO. 17901, preferably the coding region thereof, conferred the production of or the increase in gamma-tocopherol compared with the wild type control.

The nucleic acid sequence of At1g43850 from Arabidopsis thaliana, e.g. as shown in the respective line in column 5 of Table I, application no. 7, has been published in F. And the activity of the gene product thereof is the activity of transcriptional regulator.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “transcriptional regulator”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said At1g43850, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said At1g43850, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said At1g43850, or a functional equivalent or a homolog thereof as depicted in column 8 of Table II, application no. 7, preferably a homolog or functional equivalent as depicted in column 8 of Table II B, application no. 7, and being depicted in the same respective line as said At1g43850, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 18070, preferably the coding region thereof, conferred the production of or the increase in gamma-tocopherol compared with the wild type control.

The nucleic acid sequence of At1g48260 from Arabidopsis thaliana, e.g. as shown in the respective line in column 5 of Table I, application no. 7, has been published in F. And the activity of the gene product thereof is the activity of protein kinase.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said At1g48260, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said At1g48260, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said At1g48260, or a functional equivalent or a homolog thereof as depicted in column 8 of Table II, application no. 7, preferably a homolog or functional equivalent as depicted in column 8 of Table II B, application no. 7, and being depicted in the same respective line as said At1g48260, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 18235, preferably the coding region thereof, conferred the production of or the increase in gamma-tocopherol compared with the wild type control.

The nucleic acid sequence of At1g61950 from Arabidopsis thaliana, e.g. as shown in the respective line in column 5 of Table I, application no. 7, has been published in F. And the activity of the gene product thereof is the activity of calcium-dependent protein kinase.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said At1g61950, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said At1g61950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said At1g61950, or a functional equivalent or a homolog thereof as depicted in column 8 of Table II, application no. 7, preferably a homolog or functional equivalent as depicted in column 8 of Table II B, application no. 7, and being depicted in the same respective line as said At1g61950, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 18869, preferably the coding region thereof, conferred the production of or the increase in gamma-tocopherol compared with the wild type control.

The nucleic acid sequence of At1g72770 from Arabidopsis thaliana, e.g. as shown in the respective line in column 5 of Table I, application no. 7, has been published in F. And the activity of the gene product thereof is the activity of protein phosphatase.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said At1g72770, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said At1g72770, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said At1g72770, or a functional equivalent or a homolog thereof as depicted in column 8 of Table II, application no. 7, preferably a homolog or functional equivalent as depicted in column 8 of Table II B, application no. 7, and being depicted in the same respective line as said At1g72770, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19419, preferably the coding region thereof, conferred the production of or the increase in alpha-tocopherol compared with the wild type control.

The nucleic acid sequence of At2g42830 from Arabidopsis thaliana, e.g. as shown in the respective line in column 5 of Table I, application no. 7, has been published in F. And the activity of the gene product thereof is the activity of MADS box protein transcription factor.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “MADS box protein transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said At2g42830, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said At2g42830, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said At2g42830, or a functional equivalent or a homolog thereof as depicted in column 8 of Table II, application no. 7, preferably a homolog or functional equivalent as depicted in column 8 of Table II B, application no. 7, and being depicted in the same respective line as said At2g42830, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “MADS box protein transcription factor”, preferably it is the molecule of section (a) or (b) of this paragraph.

In particular, it was observed that in plants, especially in Arabidopsis thaliana, increasing or generating the activity of a gene product non-targeted with the activity of a “MADS box protein transcription factor”, preferably being encoded by a gene comprising the nucleic acid sequence SEQ ID NO. 108727, preferably the coding region thereof, conferred the production of or the increase in alpha-tocopherol compared with the wild type control.

The nucleic acid sequence of At2g47880 from Arabidopsis thaliana, e.g. as shown in the respective line in column 5 of Table I, application no. 7, has been published in F. And the activity of the gene product thereof is the activity of glutaredoxin.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said At2g47880, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said At2g47880, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said

At2g47880, or a functional equivalent or a homolog thereof as depicted in column 8 of Table II, application no. 7, preferably a homolog or functional equivalent as depicted in column 8 of Table II B, application no. 7, and being depicted in the same respective line as said At2g47880, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 21159, preferably the coding region thereof, conferred the production of or the increase in gamma-tocopherol compared with the wild type control.

The nucleic acid sequence of At3g04050 from Arabidopsis thaliana, e.g. as shown in the respective line in column 5 of Table I, application no. 7, has been published in F. And the activity of the gene product thereof is the activity of pyruvate kinase.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “pyruvate kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said At3g04050, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said At3g04050, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said At3g04050, or a functional equivalent or a homolog thereof as depicted in column 8 of Table II, application no. 7, preferably a homolog or functional equivalent as depicted in column 8 of Table II B, application no. 7, and being depicted in the same respective line as said At3g04050, and preferably the activity is increased plastidic, whereby the respective line disclose in column 7 the fine chemical gamma-tocopherol.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 21497, preferably the coding region thereof, conferred the production of or the increase in gamma-tocopherol compared with the wild type control.

The nucleic acid sequence of At3g04710 from Arabidopsis thaliana, e.g. as shown in the respective line in column 5 of Table I, application no. 7, has been published in F. And the activity of the gene product thereof is the activity of ankyrin repeat family protein.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “ankyrin repeat family protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said At3g04710, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said At3g04710, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said At3g04710, or a functional equivalent or a homolog thereof as depicted in column 8 of Table II, application no. 7, preferably a homolog or functional equivalent as depicted in column 8 of Table II B, application no. 7, and being depicted in the same respective line as said At3g04710, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “ankyrin repeat family protein”, preferably it is the molecule of section (a) or (b) of this paragraph.

In particular, it was observed that in plants, especially in Arabidopsis thaliana, increasing or generating the activity of a gene product non-targeted with the activity of a “ankyrin repeat family protein”, preferably being encoded by a gene comprising the nucleic acid sequence SEQ ID NO. 21902, preferably the coding region thereof, conferred the production of or the increase in alpha-tocopherol compared with the wild type control.

The nucleic acid sequence of At3g06270 from Arabidopsis thaliana, e.g. as shown in the respective line in column 5 of Table I, application no. 7, has been published in F. And the activity of the gene product thereof is the activity of protein phosphatase.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said At3g06270, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said At3g06270, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said At3g06270, or a functional equivalent or a homolog thereof as depicted in column 8 of Table II, application no. 7, preferably a homolog or functional equivalent as depicted in column 8 of Table II B, application no. 7, and being depicted in the same respective line as said At3g06270, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 22015, preferably the coding region thereof, conferred the production of or the increase in alpha-tocopherol compared with the wild type control.

The nucleic acid sequence of At3g08710 from Arabidopsis thaliana, e.g. as shown in the respective line in column 5 of Table I, application no. 7, has been published in F. And the activity of the gene product thereof is the activity of thioredoxin family protein.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “thioredoxin family protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said At3g08710, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said At3g08710, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said At3g08710, or a functional equivalent or a homolog thereof as depicted in column 8 of Table II, application no. 7, preferably a homolog or functional equivalent as depicted in column 8 of Table II B, application no. 7, and being depicted in the same respective line as said At3g08710, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “thioredoxin family protein”, preferably it is the molecule of section (a) or (b) of this paragraph.

In particular, it was observed that in plants, especially in Arabidopsis thaliana, increasing or generating the activity of a gene product non-targeted with the activity of a “thioredoxin family protein”, preferably being encoded by a gene comprising the nucleic acid sequence SEQ ID NO. 22249, preferably the coding region thereof, conferred the production of or the increase in alpha-tocopherol compared with the wild type control.

The nucleic acid sequence of At3g11650 from Arabidopsis thaliana, e.g. as shown in the respective line in column 5 of Table I, application no. 7, has been published in F. And the activity of the gene product thereof is the activity of harpin-induced family protein.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “harpin-induced family protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said At3g11650, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said At3g11650, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said At3g11650, or a functional equivalent or a homolog thereof as depicted in column 8 of Table II, application no. 7, preferably a homolog or functional equivalent as depicted in column 8 of Table II B, application no. 7, and being depicted in the same respective line as said At3g11650, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “harpin-induced family protein”, preferably it is the molecule of section (a) or (b) of this paragraph.

In particular, it was observed that in plants, especially in Arabidopsis thaliana, increasing or generating the activity of a gene product non-targeted with the activity of a “harpin-induced family protein”, preferably being encoded by a gene comprising the nucleic acid sequence SEQ ID NO. 22611, preferably the coding region thereof, conferred the production of or the increase in alpha-tocopherol compared with the wild type control.

The nucleic acid sequence of At3g11650 from Arabidopsis thaliana, e.g. as shown in the respective line in column 5 of Table I, application no. 7, has been published in F. And the activity of the gene product thereof is the activity of harpin-induced family protein.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “harpin-induced family protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said At3g11650, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said At3g11650, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said At3g11650, or a functional equivalent or a homolog thereof as depicted in column 8 of Table II, application no. 7, preferably a homolog or functional equivalent as depicted in column 8 of Table II B, application no. 7, and being depicted in the same respective line as said At3g11650, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “harpin-induced family protein”, preferably it is the molecule of section (a) or (b) of this paragraph.

In particular, it was observed that in plants, especially in Arabidopsis thaliana, increasing or generating the activity of a gene product non-targeted with the activity of a “harpin-induced family protein”, preferably being encoded by a gene comprising the nucleic acid sequence SEQ ID NO. 22611, preferably the coding region thereof, conferred the production of or the increase in gamma-tocopherol compared with the wild type control.

The nucleic acid sequence of At3g23000 from Arabidopsis thaliana, e.g. as shown in the respective line in column 5 of Table I, application no. 7, has been published in F. And the activity of the gene product thereof is the activity of protein kinase.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said At3g23000, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said At3g23000, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said At3g23000, or a functional equivalent or a homolog thereof as depicted in column 8 of Table II, application no. 7, preferably a homolog or functional equivalent as depicted in column 8 of Table II B, application no. 7, and being depicted in the same respective line as said At3g23000, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 1815, preferably the coding region thereof, conferred the production of or the increase in gamma-tocopherol compared with the wild type control.

The nucleic acid sequence of At3g62930 from Arabidopsis thaliana, e.g. as shown in the respective line in column 5 of Table I, application no. 7, has been published in F. And the activity of the gene product thereof is the activity of monthiol glutaredoxin.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “monthiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said At3g62930, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said At3g62930, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said At3g62930, or a functional equivalent or a homolog thereof as depicted in column 8 of Table II, application no. 7, preferably a homolog or functional equivalent as depicted in column 8 of Table II B, application no. 7, and being depicted in the same respective line as said At3g62930, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “monthiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monthiol glutaredoxin”, preferably being encoded by a gene comprising the nucleic acid sequence SEQ ID NO. 2573, preferably the coding region thereof, conferred the production of or the increase in gamma-tocopherol compared with the wild type control.

The nucleic acid sequence of At3g62950 from Arabidopsis thaliana, e.g. as shown in the respective line in column 5 of Table I, application no. 7, has been published in F. And the activity of the gene product thereof is the activity of glutaredoxin.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said At3g62950, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said At3g62950, or a functional equivalent or a homolog thereof as depicted in column 8 of Table II, application no. 7, preferably a homolog or functional equivalent as depicted in column 8 of Table II B, application no. 7, and being depicted in the same respective line as said At3g62950, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in gamma-tocopherol compared with the wild type control.

The nucleic acid sequence of At4g09960 from Arabidopsis thaliana, e.g. as shown in the respective line in column 5 of Table I, application no. 7, has been published in F. And the activity of the gene product thereof is the activity of transcription factor.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said At4g09960, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said At4g09960, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said At4g09960, or a functional equivalent or a homolog thereof as depicted in column 8 of Table II, application no. 7, preferably a homolog or functional equivalent as depicted in column 8 of Table II B, application no. 7, and being depicted in the same respective line as said At4g09960, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 109717, preferably the coding region thereof, conferred the production of or the increase in alpha-tocopherol compared with the wild type control.

The nucleic acid sequence of At4g15660 from Arabidopsis thaliana, e.g. as shown in the respective line in column 5 of Table I, application no. 7, has been published in F. And the activity of the gene product thereof is the activity of monothiol glutaredoxin.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said At4g15660, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said At4g15660, or a functional equivalent or a homolog thereof as depicted in column 8 of Table II, application no. 7, preferably a homolog or functional equivalent as depicted in column 8 of Table II B, application no. 7, and being depicted in the same respective line as said At4g15660, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in alpha-tocopherol compared with the wild type control.

The nucleic acid sequence of At4g15660 from Arabidopsis thaliana, e.g. as shown in the respective line in column 5 of Table I, application no. 7, has been published in F. And the activity of the gene product thereof is the activity of monothiol glutaredoxin.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said At4g15660, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said At4g15660, or a functional equivalent or a homolog thereof as depicted in column 8 of Table II, application no. 7, preferably a homolog or functional equivalent as depicted in column 8 of Table II B, application no. 7, and being depicted in the same respective line as said At4g15660, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in gamma-tocopherol compared with the wild type control.

The nucleic acid sequence of At4g15670 from Arabidopsis thaliana, e.g. as shown in the respective line in column 5 of Table I, application no. 7, has been published in F. And the activity of the gene product thereof is the activity of monothiol glutaredoxin.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said At4g15670, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said At4g15670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said At4g15670, or a functional equivalent or a homolog thereof as depicted in column 8 of Table II, application no. 7, preferably a homolog or functional equivalent as depicted in column 8 of Table II B, application no. 7, and being depicted in the same respective line as said At4g15670, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3279, preferably the coding region thereof, conferred the production of or the increase in alpha-tocopherol compared with the wild type control.

The nucleic acid sequence of At4g15690 from Arabidopsis thaliana, e.g. as shown in the respective line in column 5 of Table I, application no. 7, has been published in F. And the activity of the gene product thereof is the activity of monothiol glutaredoxin.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said At4g15690, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said At4g15690, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said At4g15690, or a functional equivalent or a homolog thereof as depicted in column 8 of Table II, application no. 7, preferably a homolog or functional equivalent as depicted in column 8 of Table II B, application no. 7, and being depicted in the same respective line as said At4g15690, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23844, preferably the coding region thereof, conferred the production of or the increase in alpha-tocopherol compared with the wild type control.

The nucleic acid sequence of At4g26080 from Arabidopsis thaliana, e.g. as shown in the respective line in column 5 of Table I, application no. 7, has been published in F. And the activity of the gene product thereof is the activity of protein phosphatase.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said At4g26080, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said At4g26080, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said At4g26080, or a functional equivalent or a homolog thereof as depicted in column 8 of Table II, application no. 7, preferably a homolog or functional equivalent as depicted in column 8 of Table II B, application no. 7, and being depicted in the same respective line as said At4g26080, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 68849, preferably the coding region thereof, conferred the production of or the increase in alpha-tocopherol compared with the wild type control.

The nucleic acid sequence of At4g34160 from Arabidopsis thaliana, e.g. as shown in the respective line in column 5 of Table I, application no. 7, has been published in F. And the activity of the gene product thereof is the activity of cyclin D .

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “cyclin D”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said At4g34160, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said At4g34160, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said At4g34160, or a functional equivalent or a homolog thereof as depicted in column 8 of Table II, application no. 7, preferably a homolog or functional equivalent as depicted in column 8 of Table II B, application no. 7, and being depicted in the same respective line as said At4g34160, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 24311, preferably the coding region thereof, conferred the production of or the increase in alpha-tocopherol compared with the wild type control.

The nucleic acid sequence of At4g35310 from Arabidopsis thaliana, e.g. as shown in the respective line in column 5 of Table I, application no. 7, has been published in F. And the activity of the gene product thereof is the activity of calcium-dependent protein kinase.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said At4g35310, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said At4g35310, or a functional equivalent or a homolog thereof as depicted in column 8 of Table II, application no. 7, preferably a homolog or functional equivalent as depicted in column 8 of Table II B, application no. 7, and being depicted in the same respective line as said At4g35310, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in alpha-tocopherol compared with the wild type control.

The nucleic acid sequence of At4g35310 from Arabidopsis thaliana, e.g. as shown in the respective line in column 5 of Table I, application no. 7, has been published in F. And the activity of the gene product thereof is the activity of calcium-dependent protein kinase.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said At4g35310, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said At4g35310, or a functional equivalent or a homolog thereof as depicted in column 8 of Table II, application no. 7, preferably a homolog or functional equivalent as depicted in column 8 of Table II B, application no. 7, and being depicted in the same respective line as said At4g35310, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in gamma-tocopherol compared with the wild type control.

The nucleic acid sequence of At5g60110 from Arabidopsis thaliana, e.g. as shown in the respective line in column 5 of Table I, application no. 7, has been published in F. And the activity of the gene product thereof is the activity of RNA-binding protein.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “RNA-binding protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said At5g60110, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said At5g60110, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said At5g60110, or a functional equivalent or a homolog thereof as depicted in column 8 of Table II, application no. 7, preferably a homolog or functional equivalent as depicted in column 8 of Table II B, application no. 7, and being depicted in the same respective line as said At5g60110, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “RNA-binding protein”, preferably it is the molecule of section (a) or (b) of this paragraph.

In particular, it was observed that in plants, especially in Arabidopsis thaliana, increasing or generating the activity of a gene product non-targeted with the activity of a “RNA-binding protein”, preferably being encoded by a gene comprising the nucleic acid sequence SEQ ID NO. 70006, preferably the coding region thereof, conferred the production of or the increase in alpha-tocopherol compared with the wild type control.

The nucleic acid sequence of At5g60110 from Arabidopsis thaliana, e.g. as shown in the respective line in column 5 of Table I, application no. 7, has been published in F. And the activity of the gene product thereof is the activity of RNA-binding protein.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “RNA-binding protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said At5g60110, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said At5g60110, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said At5g60110, or a functional equivalent or a homolog thereof as depicted in column 8 of Table II, application no. 7, preferably a homolog or functional equivalent as depicted in column 8 of Table II B, application no. 7, and being depicted in the same respective line as said At5g60110, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “RNA-binding protein”, preferably it is the molecule of section (a) or (b) of this paragraph.

In particular, it was observed that in plants, especially in Arabidopsis thaliana, increasing or generating the activity of a gene product non-targeted with the activity of a “RNA-binding protein”, preferably being encoded by a gene comprising the nucleic acid sequence SEQ ID NO. 70006, preferably the coding region thereof, conferred the production of or the increase in gamma-tocopherol compared with the wild type control.

The nucleic acid sequence of At5g64920 from Arabidopsis thaliana, e.g. as shown in the respective line in column 5 of Table I, application no. 7, has been published in F. And the activity of the gene product thereof is the activity of zinc finger protein.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “zinc finger protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said At5g64920, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said At5g64920, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said At5g64920, or a functional equivalent or a homolog thereof as depicted in column 8 of Table II, application no. 7, preferably a homolog or functional equivalent as depicted in column 8 of Table II B, application no. 7, and being depicted in the same respective line as said At5g64920, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “zinc finger protein”, preferably it is the molecule of section (a) or (b) of this paragraph.

In particular, it was observed that in plants, especially in Arabidopsis thaliana, increasing or generating the activity of a gene product non-targeted with the activity of a “zinc finger protein”, preferably being encoded by a gene comprising the nucleic acid sequence SEQ ID NO. 5493, preferably the coding region thereof, conferred the production of or the increase in gamma-tocopherol compared with the wild type control.

The nucleic acid sequence of AvinDRAFT_{—}3107 from Azotobacter vinelandii, e.g. as shown in the respective line in column 5 of Table I, application no. 7, has been published in D. And the activity of the gene product thereof is the activity of NAD-dependent epimerase/dehydratase.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “NAD-dependent epimerase/dehydratase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said AvinDRAFT_{—}3107, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said AvinDRAFT_{—}3107, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said AvinDRAFT_{—}3107, or a functional equivalent or a homolog thereof as depicted in column 8 of Table II, application no. 7, preferably a homolog or functional equivalent as depicted in column 8 of Table II B, application no. 7, and being depicted in the same respective line as said AvinDRAFT_{—}3107, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “NAD-dependent epimerase/dehydratase”, preferably it is the molecule of section (a) or (b) of this paragraph.

In particular, it was observed that in plants, especially in Arabidopsis thaliana, increasing or generating the activity of a gene product non-targeted with the activity of a “NAD-dependent epimerase/dehydratase”, preferably being encoded by a gene comprising the nucleic acid sequence SEQ ID NO. 110651, preferably the coding region thereof, conferred the production of or the increase in gamma-tocopherol compared with the wild type control.

The nucleic acid sequence of AvinDRAFT_{—}3209 from Azotobacter vinelandii, e.g. as shown in the respective line in column 5 of Table I, application no. 7, has been published in D. And the activity of the gene product thereof is the activity of transcriptional regulator.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “transcriptional regulator”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said AvinDRAFT_{—}3209, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said AvinDRAFT_{—}3209, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said AvinDRAFT_{—}3209, or a functional equivalent or a homolog thereof as depicted in column 8 of Table II, application no. 7, preferably a homolog or functional equivalent as depicted in column 8 of Table II B, application no. 7, and being depicted in the same respective line as said AvinDRAFT_{—}3209, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 29286, preferably the coding region thereof, conferred the production of or the increase in gamma-tocopherol compared with the wild type control.

The nucleic acid sequence of AvinDRAFT_{—}3577 from Azotobacter vinelandii, e.g. as shown in the respective line in column 5 of Table I, application no. 7, has been published in D. And the activity of the gene product thereof is the activity of ThiF family protein.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “ThiF family protein”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said AvinDRAFT_{—}3577, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said AvinDRAFT_{—}3577, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said AvinDRAFT_{—}3577, or a functional equivalent or a homolog thereof as depicted in column 8 of Table II, application no. 7, preferably a homolog or functional equivalent as depicted in column 8 of Table II B, application no. 7, and being depicted in the same respective line as said AvinDRAFT_{—}3577, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “ThiF family protein”, preferably it is the molecule of section (a) or (b) of this paragraph.

In particular, it was observed that in plants, especially in Arabidopsis thaliana, increasing or generating the activity of a gene product non-targeted with the activity of a “ThiF family protein”, preferably being encoded by a gene comprising the nucleic acid sequence SEQ ID NO. 111155, preferably the coding region thereof, conferred the production of or the increase in alpha-tocopherol compared with the wild type control.

The nucleic acid sequence of AvinDRAFT_{—}4847 from Azotobacter vinelandii, e.g. as shown in the respective line in column 5 of Table I, application no. 7, has been published in D. And the activity of the gene product thereof is the activity of isopropylmalate isomerase large subunit.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “isopropylmalate isomerase large subunit”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said AvinDRAFT_{—}4847, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said AvinDRAFT_{—}4847, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said AvinDRAFT_{—}4847, or a functional equivalent or a homolog thereof as depicted in column 8 of Table II, application no. 7, preferably a homolog or functional equivalent as depicted in column 8 of Table II B, application no. 7, and being depicted in the same respective line as said AvinDRAFT_{—}4847, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “isopropylmalate isomerase large subunit”, preferably it is the molecule of section (a) or (b) of this paragraph.

In particular, it was observed that in plants, especially in Arabidopsis thaliana, increasing or generating the activity of a gene product non-targeted with the activity of a “isopropylmalate isomerase large subunit”, preferably being encoded by a gene comprising the nucleic acid sequence SEQ ID NO. 102941, preferably the coding region thereof, conferred the production of or the increase in alpha-tocopherol compared with the wild type control.

The nucleic acid sequence of AvinDRAFT_{—}5103 from Azotobacter vinelandii, e.g. as shown in the respective line in column 5 of Table I, application no. 7, has been published in D. And the activity of the gene product thereof is the activity of hydrolase.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “hydrolase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said AvinDRAFT_{—}5103, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said AvinDRAFT_{—}5103, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said AvinDRAFT_{—}5103, or a functional equivalent or a homolog thereof as depicted in column 8 of Table II, application no. 7, preferably a homolog or functional equivalent as depicted in column 8 of Table II B, application no. 7, and being depicted in the same respective line as said AvinDRAFT_{—}5103, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “hydrolase”, preferably it is the molecule of section (a) or (b) of this paragraph.

In particular, it was observed that in plants, especially in Arabidopsis thaliana, increasing or generating the activity of a gene product non-targeted with the activity of a “hydrolase”, preferably being encoded by a gene comprising the nucleic acid sequence SEQ ID NO. 6510, preferably the coding region thereof, conferred the production of or the increase in alpha-tocopherol compared with the wild type control.

The nucleic acid sequence of AvinDRAFT_{—}5103 from Azotobacter vinelandii, e.g. as shown in the respective line in column 5 of Table I, application no. 7, has been published in D. And the activity of the gene product thereof is the activity of hydrolase.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “hydrolase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said AvinDRAFT_{—}5103, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said AvinDRAFT_{—}5103, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said AvinDRAFT _{—}5103, or a functional equivalent or a homolog thereof as depicted in column 8 of Table II, application no. 7, preferably a homolog or functional equivalent as depicted in column 8 of Table II B, application no. 7, and being depicted in the same respective line as said AvinDRAFT_{—}5103, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “hydrolase”, preferably it is the molecule of section (a) or (b) of this paragraph.

In particular, it was observed that in plants, especially in Arabidopsis thaliana, increasing or generating the activity of a gene product non-targeted with the activity of a “hydrolase”, preferably being encoded by a gene comprising the nucleic acid sequence SEQ ID NO. 6510, preferably the coding region thereof, conferred the production of or the increase in gamma-tocopherol compared with the wild type control.

The nucleic acid sequence of AvinDRAFT_{—}6679 from Azotobacter vinelandii, e.g. as shown in the respective line in column 5 of Table I, application no. 7, has been published in D. And the activity of the gene product thereof is the activity of electron transfer flavoprotein subunit beta.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “electron transfer flavoprotein subunit beta”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said AvinDRAFT_{—}6679, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said AvinDRAFT_{—}6679, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said AvinDRAFT_{—}6679, or a functional equivalent or a homolog thereof as depicted in column 8 of Table II, application no. 7, preferably a homolog or functional equivalent as depicted in column 8 of Table II B, application no. 7, and being depicted in the same respective line as said AvinDRAFT_{—}6679, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “electron transfer flavoprotein subunit beta”, preferably it is the molecule of section (a) or (b) of this paragraph.

In particular, it was observed that in plants, especially in Arabidopsis thaliana, increasing or generating the activity of a gene product non-targeted with the activity of a “electron transfer flavoprotein subunit beta”, preferably being encoded by a gene comprising the nucleic acid sequence SEQ ID NO. 74729, preferably the coding region thereof, conferred the production of or the increase in gamma-tocopherol compared with the wild type control.

The nucleic acid sequence of B0004 from Escherichia coli, e.g. as shown in the respective line in column 5 of Table I, application no. 7, has been published in B. And the activity of the gene product thereof is the activity of threonine synthase.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “threonine synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said B0004, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B0004, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B0004, or a functional equivalent or a homolog thereof as depicted in column 8 of Table II, application no. 7, preferably a homolog or functional equivalent as depicted in column 8 of Table II B, application no. 7, and being depicted in the same respective line as said B0004, and preferably the activity is increased plastidic, whereby the respective line disclose in column 7 the fine chemical gamma-tocopherol.

Accordingly, in one embodiment, the molecule which activity is to be increased in the process of the invention is the gene product with an activity as a “threonine synthase”, preferably it is the molecule of section (a) or (b) of this paragraph.

In particular, it was observed that in plants, especially in Arabidopsis thaliana, increasing or generating the activity of a gene product plastidic with the activity of a “threonine synthase”, preferably being encoded by a gene comprising the nucleic acid sequence SEQ ID NO. 34889, preferably the coding region thereof, conferred the production of or the increase in gamma-tocopherol compared with the wild type control.

The nucleic acid sequence of B0124 from Escherichia coli, e.g. as shown in the respective line in column 5 of Table I, application no. 7, has been published in B. And the activity of the gene product thereof is the activity of glucose dehydrogenase.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “glucose dehydrogenase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said B0124, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B0124, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B0124, or a functional equivalent or a homolog thereof as depicted in column 8 of Table II, application no. 7, preferably a homolog or functional equivalent as depicted in column 8 of Table II B, application no. 7, and being depicted in the same respective line as said B0124, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “glucose dehydrogenase”, preferably it is the molecule of section (a) or (b) of this paragraph.

In particular, it was observed that in plants, especially in Arabidopsis thaliana, increasing or generating the activity of a gene product non-targeted with the activity of a “glucose dehydrogenase”, preferably being encoded by a gene comprising the nucleic acid sequence SEQ ID NO. 35482, preferably the coding region thereof, conferred the production of or the increase in alpha-tocopherol compared with the wild type control.

The nucleic acid sequence of B0124 from Escherichia coli, e.g. as shown in the respective line in column 5 of Table I, application no. 7, has been published in B. And the activity of the gene product thereof is the activity of glucose dehydrogenase.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “glucose dehydrogenase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said B0124, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B0124, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B0124, or a functional equivalent or a homolog thereof as depicted in column 8 of Table II, application no. 7, preferably a homolog or functional equivalent as depicted in column 8 of Table II B, application no. 7, and being depicted in the same respective line as said B0124, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “glucose dehydrogenase”, preferably it is the molecule of section (a) or (b) of this paragraph.

In particular, it was observed that in plants, especially in Arabidopsis thaliana, increasing or generating the activity of a gene product non-targeted with the activity of a “glucose dehydrogenase”, preferably being encoded by a gene comprising the nucleic acid sequence SEQ ID NO. 35482, preferably the coding region thereof, conferred the production of or the increase in gamma-tocopherol compared with the wild type control.

The nucleic acid sequence of B0161 from Escherichia coli, e.g. as shown in the respective line in column 5 of Table I, application no. 7, has been published in B. And the activity of the gene product thereof is the activity of serine protease.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “serine protease”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said B0161, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B0161, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B0161, or a functional equivalent or a homolog thereof as depicted in column 8 of Table II, application no. 7, preferably a homolog or functional equivalent as depicted in column 8 of Table II B, application no. 7, and being depicted in the same respective line as said B0161, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “serine protease”, preferably it is the molecule of section (a) or (b) of this paragraph.

In particular, it was observed that in plants, especially in Arabidopsis thaliana, increasing or generating the activity of a gene product non-targeted with the activity of a “serine protease”, preferably being encoded by a gene comprising the nucleic acid sequence SEQ ID NO. 7081, preferably the coding region thereof, conferred the production of or the increase in gamma-tocopherol compared with the wild type control.

The nucleic acid sequence of B0421 from Escherichia coli, e.g. as shown in the respective line in column 5 of Table I, application no. 7, has been published in B. And the activity of the gene product thereof is the activity of geranyltranstransferase.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “geranyltranstransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said B0421, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B0421, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B0421, or a functional equivalent or a homolog thereof as depicted in column 8 of Table II, application no. 7, preferably a homolog or functional equivalent as depicted in column 8 of Table II B, application no. 7, and being depicted in the same respective line as said B0421, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “geranyltranstransferase”, preferably it is the molecule of section (a) or (b) of this paragraph.

In particular, it was observed that in plants, especially in Arabidopsis thaliana, increasing or generating the activity of a gene product non-targeted with the activity of a “geranyltranstransferase”, preferably being encoded by a gene comprising the nucleic acid sequence SEQ ID NO. 111340, preferably the coding region thereof, conferred the production of or the increase in alpha-tocopherol compared with the wild type control.

The nucleic acid sequence of B0449 from Escherichia coli, e.g. as shown in the respective line in column 5 of Table I, application no. 7, has been published in B. And the activity of the gene product thereof is the activity of ATP-binding component of a transport system.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “ATP-binding component of a transport system”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said B0449, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B0449, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B0449, or a functional equivalent or a homolog thereof as depicted in column 8 of Table II, application no. 7, preferably a homolog or functional equivalent as depicted in column 8 of Table II B, application no. 7, and being depicted in the same respective line as said B0449, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “ATP-binding component of a transport system”, preferably it is the molecule of section (a) or (b) of this paragraph.

In particular, it was observed that in plants, especially in Arabidopsis thaliana, increasing or generating the activity of a gene product non-targeted with the activity of a “ATP-binding component of a transport system”, preferably being encoded by a gene comprising the nucleic acid sequence SEQ ID NO. 7333, preferably the coding region thereof, conferred the production of or the increase in gamma-tocopherol compared with the wild type control.

The nucleic acid sequence of B0754 from Escherichia coli, e.g. as shown in the respective line in column 5 of Table I, application no. 7, has been published in B. And the activity of the gene product thereof is the activity of 3-deoxy-D-arabinoheptulosonate-7-phosphatesynthase.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “3-deoxy-D-arabinoheptulosonate-7-phosphatesynthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said B0754, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B0754, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B0754, or a functional equivalent or a homolog thereof as depicted in column 8 of Table II, application no. 7, preferably a homolog or functional equivalent as depicted in column 8 of Table II B, application no. 7, and being depicted in the same respective line as said B0754, and preferably the activity is increased plastidic, whereby the respective line disclose in column 7 the fine chemical alpha-tocopherol.

Accordingly, in one embodiment, the molecule which activity is to be increased in the process of the invention is the gene product with an activity as a “3-deoxy-D-arabinoheptulosonate-7-phosphatesynthase”, preferably it is the molecule of section (a) or (b) of this paragraph.

In particular, it was observed that in plants, especially in Arabidopsis thaliana, increasing or generating the activity of a gene product plastidic with the activity of a “3-deoxy-D-arabinoheptulosonate-7-phosphatesynthase”, preferably being encoded by a gene comprising the nucleic acid sequence SEQ ID NO. 75286, preferably the coding region thereof, conferred the production of or the increase in alpha-tocopherol compared with the wild type control.

The nucleic acid sequence of B0898 from Escherichia coli, e.g. as shown in the respective line in column 5 of Table I, application no. 7, has been published in B. And the activity of the gene product thereof is the activity of major facilitator superfamily transporter protein.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “major facilitator superfamily transporter protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said B0898, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B0898, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B0898, or a functional equivalent or a homolog thereof as depicted in column 8 of Table II, application no. 7, preferably a homolog or functional equivalent as depicted in column 8 of Table II B, application no. 7, and being depicted in the same respective line as said B0898, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “major facilitator superfamily transporter protein”, preferably it is the molecule of section (a) or (b) of this paragraph.

In particular, it was observed that in plants, especially in Arabidopsis thaliana, increasing or generating the activity of a gene product non-targeted with the activity of a “major facilitator superfamily transporter protein”, preferably being encoded by a gene comprising the nucleic acid sequence SEQ ID NO. 7917, preferably the coding region thereof, conferred the production of or the increase in gamma-tocopherol compared with the wild type control.

The nucleic acid sequence of B1163 from Escherichia coli, e.g. as shown in the respective line in column 5 of Table I, application no. 7, has been published in B. And the activity of the gene product thereof is the activity of b1163-protein.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “b1163-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said B1163, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B1163, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B1163, or a functional equivalent or a homolog thereof as depicted in column 8 of Table II, application no. 7, preferably a homolog or functional equivalent as depicted in column 8 of Table II B, application no. 7, and being depicted in the same respective line as said B1163, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “b1163-protein”, preferably it is the molecule of section (a) or (b) of this paragraph.

In particular, it was observed that in plants, especially in Arabidopsis thaliana, increasing or generating the activity of a gene product non-targeted with the activity of a “b1163-protein”, preferably being encoded by a gene comprising the nucleic acid sequence SEQ ID NO. 37394, preferably the coding region thereof, conferred the production of or the increase in alpha-tocopherol compared with the wild type control.

The nucleic acid sequence of B1183 from Escherichia coli, e.g. as shown in the respective line in column 5 of Table I, application no. 7, has been published in B. And the activity of the gene product thereof is the activity of DNA polymerase subunit.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “DNA polymerase subunit”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said B1183, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B1183, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B1183, or a functional equivalent or a homolog thereof as depicted in column 8 of Table II, application no. 7, preferably a homolog or functional equivalent as depicted in column 8 of Table II B, application no. 7, and being depicted in the same respective line as said B1183, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “DNA polymerase subunit”, preferably it is the molecule of section (a) or (b) of this paragraph.

In particular, it was observed that in plants, especially in Arabidopsis thaliana, increasing or generating the activity of a gene product non-targeted with the activity of a “DNA polymerase subunit”, preferably being encoded by a gene comprising the nucleic acid sequence SEQ ID NO. 111910, preferably the coding region thereof, conferred the production of or the increase in gamma-tocopherol compared with the wild type control.

The nucleic acid sequence of B1219 from Escherichia coli, e.g. as shown in the respective line in column 5 of Table I, application no. 7, has been published in B. And the activity of the gene product thereof is the activity of b1219-protein.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “b1219-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said B1219, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B1219, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B1219, or a functional equivalent or a homolog thereof as depicted in column 8 of Table II, application no. 7, preferably a homolog or functional equivalent as depicted in column 8 of Table II B, application no. 7, and being depicted in the same respective line as said B1219, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “b1219-protein”, preferably it is the molecule of section (a) or (b) of this paragraph.

In particular, it was observed that in plants, especially in Arabidopsis thaliana, increasing or generating the activity of a gene product non-targeted with the activity of a “b1219-protein”, preferably being encoded by a gene comprising the nucleic acid sequence SEQ ID NO. 112072, preferably the coding region thereof, conferred the production of or the increase in alpha-tocopherol compared with the wild type control.

The nucleic acid sequence of B1234 from Escherichia coli, e.g. as shown in the respective line in column 5 of Table I, application no. 7, has been published in B. And the activity of the gene product thereof is the activity of b1234-protein.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “b1234-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said B1234, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B1234, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B1234, or a functional equivalent or a homolog thereof as depicted in column 8 of Table II, application no. 7, preferably a homolog or functional equivalent as depicted in column 8 of Table II B, application no. 7, and being depicted in the same respective line as said B1234, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “b1234-protein”, preferably it is the molecule of section (a) or (b) of this paragraph.

In particular, it was observed that in plants, especially in Arabidopsis thaliana, increasing or generating the activity of a gene product non-targeted with the activity of a “b1234-protein”, preferably being encoded by a gene comprising the nucleic acid sequence SEQ ID NO. 112111, preferably the coding region thereof, conferred the production of or the increase in gamma-tocopherol compared with the wild type control.

The nucleic acid sequence of B1479 from Escherichia coli, e.g. as shown in the respective line in column 5 of Table I, application no. 7, has been published in B. And the activity of the gene product thereof is the activity of NADP-dependent malic enzyme.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “NADP-dependent malic enzyme”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said B1479, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B1479, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B1479, or a functional equivalent or a homolog thereof as depicted in column 8 of Table II, application no. 7, preferably a homolog or functional equivalent as depicted in column 8 of Table II B, application no. 7, and being depicted in the same respective line as said B1479, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “NADP-dependent malic enzyme”, preferably it is the molecule of section (a) or (b) of this paragraph.

In particular, it was observed that in plants, especially in Arabidopsis thaliana, increasing or generating the activity of a gene product non-targeted with the activity of a “NADP-dependent malic enzyme”, preferably being encoded by a gene comprising the nucleic acid sequence SEQ ID NO. 77774, preferably the coding region thereof, conferred the production of or the increase in gamma-tocopherol compared with the wild type control.

The nucleic acid sequence of B1522 from Escherichia coli, e.g. as shown in the respective line in column 5 of Table I, application no. 7, has been published in B. And the activity of the gene product thereof is the activity of b1522-protein.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “b1522-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said B1522, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B1522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B1522, or a functional equivalent or a homolog thereof as depicted in column 8 of Table II, application no. 7, preferably a homolog or functional equivalent as depicted in column 8 of Table II B, application no. 7, and being depicted in the same respective line as said B1522, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “b1522-protein”, preferably it is the molecule of section (a) or (b) of this paragraph.

In particular, it was observed that in plants, especially in Arabidopsis thaliana, increasing or generating the activity of a gene product non-targeted with the activity of a “b1522-protein”, preferably being encoded by a gene comprising the nucleic acid sequence SEQ ID NO. 7947, preferably the coding region thereof, conferred the production of or the increase in gamma-tocopherol compared with the wild type control.

The nucleic acid sequence of B1597 from Escherichia coli, e.g. as shown in the respective line in column 5 of Table I, application no. 7, has been published in B. And the activity of the gene product thereof is the activity of acid shock protein.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “acid shock protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said B1597, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B1597, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B1597, or a functional equivalent or a homolog thereof as depicted in column 8 of Table II, application no. 7, preferably a homolog or functional equivalent as depicted in column 8 of Table II B, application no. 7, and being depicted in the same respective line as said B1597, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “acid shock protein”, preferably it is the molecule of section (a) or (b) of this paragraph.

In particular, it was observed that in plants, especially in Arabidopsis thaliana, increasing or generating the activity of a gene product non-targeted with the activity of a “acid shock protein”, preferably being encoded by a gene comprising the nucleic acid sequence SEQ ID NO. 38300, preferably the coding region thereof, conferred the production of or the increase in alpha-tocopherol compared with the wild type control.

The nucleic acid sequence of B1601 from Escherichia coli, e.g. as shown in the respective line in column 5 of Table I, application no. 7, has been published in B. And the activity of the gene product thereof is the activity of transport protein.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “transport protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said B1601, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B1601, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B1601, or a functional equivalent or a homolog thereof as depicted in column 8 of Table II, application no. 7, preferably a homolog or functional equivalent as depicted in column 8 of Table II B, application no. 7, and being depicted in the same respective line as said B1601, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “transport protein”, preferably it is the molecule of section (a) or (b) of this paragraph.

In particular, it was observed that in plants, especially in Arabidopsis thaliana, increasing or generating the activity of a gene product non-targeted with the activity of a “transport protein”, preferably being encoded by a gene comprising the nucleic acid sequence SEQ ID NO. 7992, preferably the coding region thereof, conferred the production of or the increase in alpha-tocopherol compared with the wild type control.

The nucleic acid sequence of B1897 from Escherichia coli, e.g. as shown in the respective line in column 5 of Table I, application no. 7, has been published in B. And the activity of the gene product thereof is the activity of trehalose-phosphatase.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “trehalose-phosphatase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said B1897, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B1897, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B1897, or a functional equivalent or a homolog thereof as depicted in column 8 of Table II, application no. 7, preferably a homolog or functional equivalent as depicted in column 8 of Table II B, application no. 7, and being depicted in the same respective line as said B1897, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “trehalose-phosphatase”, preferably it is the molecule of section (a) or (b) of this paragraph.

In particular, it was observed that in plants, especially in Arabidopsis thaliana, increasing or generating the activity of a gene product non-targeted with the activity of a “trehalosephosphatase”, preferably being encoded by a gene comprising the nucleic acid sequence SEQ ID NO. 79056, preferably the coding region thereof, conferred the production of or the increase in gamma-tocopherol compared with the wild type control.

The nucleic acid sequence of B1921 from Escherichia coli, e.g. as shown in the respective line in column 5 of Table I, application no. 7, has been published in B. And the activity of the gene product thereof is the activity of b1921-protein.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “b1921-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said B1921, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B1921, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B1921, or a functional equivalent or a homolog thereof as depicted in column 8 of Table II, application no. 7, preferably a homolog or functional equivalent as depicted in column 8 of Table II B, application no. 7, and being depicted in the same respective line as said B1921, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “b1921-protein”, preferably it is the molecule of section (a) or (b) of this paragraph.

In particular, it was observed that in plants, especially in Arabidopsis thaliana, increasing or generating the activity of a gene product non-targeted with the activity of a “b1921-protein”, preferably being encoded by a gene comprising the nucleic acid sequence SEQ ID NO. 112169, preferably the coding region thereof, conferred the production of or the increase in gamma-tocopherol compared with the wild type control.

The nucleic acid sequence of B2027 from Escherichia coli, e.g. as shown in the respective line in column 5 of Table I, application no. 7, has been published in B. And the activity of the gene product thereof is the activity of O-antigen chain length determinant.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “O-antigen chain length determinant”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said B2027, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B2027, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B2027, or a functional equivalent or a homolog thereof as depicted in column 8 of Table II, application no. 7, preferably a homolog or functional equivalent as depicted in column 8 of Table II B, application no. 7, and being depicted in the same respective line as said B2027, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “O-antigen chain length determinant”, preferably it is the molecule of section (a) or (b) of this paragraph.

In particular, it was observed that in plants, especially in Arabidopsis thaliana, increasing or generating the activity of a gene product non-targeted with the activity of a “O-antigen chain length determinant”, preferably being encoded by a gene comprising the nucleic acid sequence SEQ ID NO. 79217, preferably the coding region thereof, conferred the production of or the increase in gamma-tocopherol compared with the wild type control.

The nucleic acid sequence of B2043 from Escherichia coli, e.g. as shown in the respective line in column 5 of Table I, application no. 7, has been published in B. And the activity of the gene product thereof is the activity of colanic acid biosynthesis protein.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “colanic acid biosynthesis protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said B2043, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B2043, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B2043, or a functional equivalent or a homolog thereof as depicted in column 8 of Table II, application no. 7, preferably a homolog or functional equivalent as depicted in column 8 of Table II B, application no. 7, and being depicted in the same respective line as said B2043, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “colanic acid biosynthesis protein”, preferably it is the molecule of section (a) or (b) of this paragraph.

In particular, it was observed that in plants, especially in Arabidopsis thaliana, increasing or generating the activity of a gene product non-targeted with the activity of a “colanic acid biosynthesis protein”, preferably being encoded by a gene comprising the nucleic acid sequence SEQ ID NO. 112193, preferably the coding region thereof, conferred the production of or the increase in alpha-tocopherol compared with the wild type control.

The nucleic acid sequence of B2360 from Escherichia coli, e.g. as shown in the respective line in column 5 of Table I, application no. 7, has been published in B. And the activity of the gene product thereof is the activity of b2360-protein.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “b2360-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said B2360, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B2360, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B2360, or a functional equivalent or a homolog thereof as depicted in column 8 of Table II, application no. 7, preferably a homolog or functional equivalent as depicted in column 8 of Table II B, application no. 7, and being depicted in the same respective line as said B2360, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “b2360-protein”, preferably it is the molecule of section (a) or (b) of this paragraph.

In particular, it was observed that in plants, especially in Arabidopsis thaliana, increasing or generating the activity of a gene product non-targeted with the activity of a “b2360-protein”, preferably being encoded by a gene comprising the nucleic acid sequence SEQ ID NO. 39219, preferably the coding region thereof, conferred the production of or the increase in gamma-tocopherol compared with the wild type control.

The nucleic acid sequence of B2546 from Escherichia coli, e.g. as shown in the respective line in column 5 of Table I, application no. 7, has been published in B. And the activity of the gene product thereof is the activity of putative transport system permease protein.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “putative transport system permease protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said B2546, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B2546, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B2546, or a functional equivalent or a homolog thereof as depicted in column 8 of Table II, application no. 7, preferably a homolog or functional equivalent as depicted in column 8 of Table II B, application no. 7, and being depicted in the same respective line as said B2546, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “putative transport system permease protein”, preferably it is the molecule of section (a) or (b) of this paragraph.

In particular, it was observed that in plants, especially in Arabidopsis thaliana, increasing or generating the activity of a gene product non-targeted with the activity of a “putative transport system permease protein”, preferably being encoded by a gene comprising the nucleic acid sequence SEQ ID NO. 99898, preferably the coding region thereof, conferred the production of or the increase in alpha-tocopherol compared with the wild type control.

The nucleic acid sequence of B2548 from Escherichia coli, e.g. as shown in the respective line in column 5 of Table I, application no. 7, has been published in B. And the activity of the gene product thereof is the activity of b2548-protein.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “b2548-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said B2548, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B2548, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B2548, or a functional equivalent or a homolog thereof as depicted in column 8 of Table II, application no. 7, preferably a homolog or functional equivalent as depicted in column 8 of Table II B, application no. 7, and being depicted in the same respective line as said B2548, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “b2548-protein”, preferably it is the molecule of section (a) or (b) of this paragraph.

In particular, it was observed that in plants, especially in Arabidopsis thaliana, increasing or generating the activity of a gene product non-targeted with the activity of a “b2548-protein”, preferably being encoded by a gene comprising the nucleic acid sequence SEQ ID NO. 40637, preferably the coding region thereof, conferred the production of or the increase in gamma-tocopherol compared with the wild type control.

The nucleic acid sequence of B2613 from Escherichia coli, e.g. as shown in the respective line in column 5 of Table I, application no. 7, has been published in B. And the activity of the gene product thereof is the activity of b2613-protein.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “b2613-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said B2613, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B2613, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B2613, or a functional equivalent or a homolog thereof as depicted in column 8 of Table II, application no. 7, preferably a homolog or functional equivalent as depicted in column 8 of Table II B, application no. 7, and being depicted in the same respective line as said B2613, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “b2613-protein”, preferably it is the molecule of section (a) or (b) of this paragraph.

In particular, it was observed that in plants, especially in Arabidopsis thaliana, increasing or generating the activity of a gene product non-targeted with the activity of a “b2613-protein”, preferably being encoded by a gene comprising the nucleic acid sequence SEQ ID NO. 40665, preferably the coding region thereof, conferred the production of or the increase in gamma-tocopherol compared with the wild type control.

The nucleic acid sequence of B2673 from Escherichia coli, e.g. as shown in the respective line in column 5 of Table I, application no. 7, has been published in B. And the activity of the gene product thereof is the activity of b2673-protein.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “b2673-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said B2673, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B2673, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B2673, or a functional equivalent or a homolog thereof as depicted in column 8 of Table II, application no. 7, preferably a homolog or functional equivalent as depicted in column 8 of Table II B, application no. 7, and being depicted in the same respective line as said B2673, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “b2673-protein”, preferably it is the molecule of section (a) or (b) of this paragraph.

In particular, it was observed that in plants, especially in Arabidopsis thaliana, increasing or generating the activity of a gene product non-targeted with the activity of a “b2673-protein”, preferably being encoded by a gene comprising the nucleic acid sequence SEQ ID NO. 9244, preferably the coding region thereof, conferred the production of or the increase in gamma-tocopherol compared with the wild type control.

The nucleic acid sequence of B2701 from Escherichia coli, e.g. as shown in the respective line in column 5 of Table I, application no. 7, has been published in B. And the activity of the gene product thereof is the activity of murein transglycosylase.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “murein transglycosylase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said B2701, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B2701, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B2701, or a functional equivalent or a homolog thereof as depicted in column 8 of Table II, application no. 7, preferably a homolog or functional equivalent as depicted in column 8 of Table II B, application no. 7, and being depicted in the same respective line as said B2701, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “murein transglycosylase”, preferably it is the molecule of section (a) or (b) of this paragraph.

In particular, it was observed that in plants, especially in Arabidopsis thaliana, increasing or generating the activity of a gene product non-targeted with the activity of a “murein transglycosylase”, preferably being encoded by a gene comprising the nucleic acid sequence SEQ ID NO. 80756, preferably the coding region thereof, conferred the production of or the increase in gamma-tocopherol compared with the wild type control.

The nucleic acid sequence of B2812 from Escherichia coli, e.g. as shown in the respective line in column 5 of Table I, application no. 7, has been published in B. And the activity of the gene product thereof is the activity of b2812-protein.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “b2812-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said B2812, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B2812, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B2812, or a functional equivalent or a homolog thereof as depicted in column 8 of Table II, application no. 7, preferably a homolog or functional equivalent as depicted in column 8 of Table II B, application no. 7, and being depicted in the same respective line as said B2812, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “b2812-protein”, preferably it is the molecule of section (a) or (b) of this paragraph.

In particular, it was observed that in plants, especially in Arabidopsis thaliana, increasing or generating the activity of a gene product non-targeted with the activity of a “b2812-protein”, preferably being encoded by a gene comprising the nucleic acid sequence SEQ ID NO. 40795, preferably the coding region thereof, conferred the production of or the increase in gamma-tocopherol compared with the wild type control.

The nucleic acid sequence of B2818 from Escherichia coli, e.g. as shown in the respective line in column 5 of Table I, application no. 7, has been published in B. And the activity of the gene product thereof is the activity of amino-acid acetyltransferase.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “amino-acid acetyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said B2818, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B2818, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B2818, or a functional equivalent or a homolog thereof as depicted in column 8 of Table II, application no. 7, preferably a homolog or functional equivalent as depicted in column 8 of Table II B, application no. 7, and being depicted in the same respective line as said B2818, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “amino-acid acetyltransferase”, preferably it is the molecule of section (a) or (b) of this paragraph.

In particular, it was observed that in plants, especially in Arabidopsis thaliana, increasing or generating the activity of a gene product non-targeted with the activity of a “amino-acid acetyltransferase”, preferably being encoded by a gene comprising the nucleic acid sequence SEQ ID NO. 81576, preferably the coding region thereof, conferred the production of or the increase in alpha-tocopherol compared with the wild type control.

The nucleic acid sequence of B2818 from Escherichia coli, e.g. as shown in the respective line in column 5 of Table I, application no. 7, has been published in B. And the activity of the gene product thereof is the activity of amino-acid acetyltransferase.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “amino-acid acetyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said B2818, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B2818, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B2818, or a functional equivalent or a homolog thereof as depicted in column 8 of Table II, application no. 7, preferably a homolog or functional equivalent as depicted in column 8 of Table II B, application no. 7, and being depicted in the same respective line as said B2818, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “amino-acid acetyltransferase”, preferably it is the molecule of section (a) or (b) of this paragraph.

In particular, it was observed that in plants, especially in Arabidopsis thaliana, increasing or generating the activity of a gene product non-targeted with the activity of a “amino-acid acetyltransferase”, preferably being encoded by a gene comprising the nucleic acid sequence SEQ ID NO. 81576, preferably the coding region thereof, conferred the production of or the increase in gamma-tocopherol compared with the wild type control.

The nucleic acid sequence of B2977 from Escherichia coli, e.g. as shown in the respective line in column 5 of Table I, application no. 7, has been published in B. And the activity of the gene product thereof is the activity of glycolate utilization protein.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “glycolate utilization protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said B2977, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B2977, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B2977, or a functional equivalent or a homolog thereof as depicted in column 8 of Table II, application no. 7, preferably a homolog or functional equivalent as depicted in column 8 of Table II B, application no. 7, and being depicted in the same respective line as said B2977, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “glycolate utilization protein”, preferably it is the molecule of section (a) or (b) of this paragraph.

In particular, it was observed that in plants, especially in Arabidopsis thaliana, increasing or generating the activity of a gene product non-targeted with the activity of a “glycolate utilization protein”, preferably being encoded by a gene comprising the nucleic acid sequence SEQ ID NO. 112216, preferably the coding region thereof, conferred the production of or the increase in alpha-tocopherol compared with the wild type control.

The nucleic acid sequence of B3194 from Escherichia coli, e.g. as shown in the respective line in column 5 of Table I, application no. 7, has been published in B. And the activity of the gene product thereof is the activity of ABC transporter permease subunit.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “ABC transporter permease subunit”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said B3194, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B3194, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B3194, or a functional equivalent or a homolog thereof as depicted in column 8 of Table II, application no. 7, preferably a homolog or functional equivalent as depicted in column 8 of Table II B, application no. 7, and being depicted in the same respective line as said B3194, and preferably the activity is increased plastidic, whereby the respective line disclose in column 7 the fine chemical alpha-tocopherol.

Accordingly, in one embodiment, the molecule which activity is to be increased in the process of the invention is the gene product with an activity as a “ABC transporter permease subunit”, preferably it is the molecule of section (a) or (b) of this paragraph.

In particular, it was observed that in plants, especially in Arabidopsis thaliana, increasing or generating the activity of a gene product plastidic with the activity of a “ABC transporter permease subunit”, preferably being encoded by a gene comprising the nucleic acid sequence SEQ ID NO. 112380, preferably the coding region thereof, conferred the production of or the increase in alpha-tocopherol compared with the wild type control.

The nucleic acid sequence of B3790 from Escherichia coli, e.g. as shown in the respective line in column 5 of Table I, application no. 7, has been published in B. And the activity of the gene product thereof is the activity of lipopolysaccharide biosynthesis protein.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “lipopolysaccharide biosynthesis protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said B3790, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B3790, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B3790, or a functional equivalent or a homolog thereof as depicted in column 8 of Table II, application no. 7, preferably a homolog or functional equivalent as depicted in column 8 of Table II B, application no. 7, and being depicted in the same respective line as said B3790, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “lipopolysaccharide biosynthesis protein”, preferably it is the molecule of section (a) or (b) of this paragraph.

In particular, it was observed that in plants, especially in Arabidopsis thaliana, increasing or generating the activity of a gene product non-targeted with the activity of a “lipopolysaccharide biosynthesis protein”, preferably being encoded by a gene comprising the nucleic acid sequence SEQ ID NO. 112606, preferably the coding region thereof, conferred the production of or the increase in gamma-tocopherol compared with the wild type control.

The nucleic acid sequence of B3890 from Escherichia coli, e.g. as shown in the respective line in column 5 of Table I, application no. 7, has been published in B. And the activity of the gene product thereof is the activity of b3890-protein.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “b3890-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said B3890, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B3890, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B3890, or a functional equivalent or a homolog thereof as depicted in column 8 of Table II, application no. 7, preferably a homolog or functional equivalent as depicted in column 8 of Table II B, application no. 7, and being depicted in the same respective line as said B3890, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “b3890-protein”, preferably it is the molecule of section (a) or (b) of this paragraph.

In particular, it was observed that in plants, especially in Arabidopsis thaliana, increasing or generating the activity of a gene product non-targeted with the activity of a “b3890-protein”, preferably being encoded by a gene comprising the nucleic acid sequence SEQ ID NO. 112634, preferably the coding region thereof, conferred the production of or the increase in alpha-tocopherol compared with the wild type control.

The nucleic acid sequence of B3945 from Escherichia coli, e.g. as shown in the respective line in column 5 of Table I, application no. 7, has been published in B. And the activity of the gene product thereof is the activity of glycerol dehydrogenase.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “glycerol dehydrogenase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said B3945, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B3945, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B3945, or a functional equivalent or a homolog thereof as depicted in column 8 of Table II, application no. 7, preferably a homolog or functional equivalent as depicted in column 8 of Table II B, application no. 7, and being depicted in the same respective line as said B3945, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “glycerol dehydrogenase”, preferably it is the molecule of section (a) or (b) of this paragraph.

In particular, it was observed that in plants, especially in Arabidopsis thaliana, increasing or generating the activity of a gene product non-targeted with the activity of a “glycerol dehydrogenase”, preferably being encoded by a gene comprising the nucleic acid sequence SEQ ID NO. 44223, preferably the coding region thereof, conferred the production of or the increase in alpha-tocopherol compared with the wild type control.

The nucleic acid sequence of B3989 from Escherichia coli, e.g. as shown in the respective line in column 5 of Table I, application no. 7, has been published in B. And the activity of the gene product thereof is the activity of b3989-protein.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “b3989-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said B3989, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B3989, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B3989, or a functional equivalent or a homolog thereof as depicted in column 8 of Table II, application no. 7, preferably a homolog or functional equivalent as depicted in column 8 of Table II B, application no. 7, and being depicted in the same respective line as said B3989, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “b3989-protein”, preferably it is the molecule of section (a) or (b) of this paragraph.

In particular, it was observed that in plants, especially in Arabidopsis thaliana, increasing or generating the activity of a gene product non-targeted with the activity of a “b3989-protein”, preferably being encoded by a gene comprising the nucleic acid sequence SEQ ID NO. 44372, preferably the coding region thereof, conferred the production of or the increase in alpha-tocopherol compared with the wild type control.

The nucleic acid sequence of B3989 from Escherichia coli, e.g. as shown in the respective line in column 5 of Table I, application no. 7, has been published in B. And the activity of the gene product thereof is the activity of b3989-protein.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “b3989-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said B3989, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B3989, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B3989, or a functional equivalent or a homolog thereof as depicted in column 8 of Table II, application no. 7, preferably a homolog or functional equivalent as depicted in column 8 of Table II B, application no. 7, and being depicted in the same respective line as said B3989, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “b3989-protein”, preferably it is the molecule of section (a) or (b) of this paragraph.

In particular, it was observed that in plants, especially in Arabidopsis thaliana, increasing or generating the activity of a gene product non-targeted with the activity of a “b3989-protein”, preferably being encoded by a gene comprising the nucleic acid sequence SEQ ID NO. 44372, preferably the coding region thereof, conferred the production of or the increase in gamma-tocopherol compared with the wild type control.

The nucleic acid sequence of B4029 from Escherichia coli, e.g. as shown in the respective line in column 5 of Table I, application no. 7, has been published in B. And the activity of the gene product thereof is the activity of b4029-protein.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “b4029-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said B4029, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B4029, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B4029, or a functional equivalent or a homolog thereof as depicted in column 8 of Table II, application no. 7, preferably a homolog or functional equivalent as depicted in column 8 of Table II B, application no. 7, and being depicted in the same respective line as said B4029, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “b4029-protein”, preferably it is the molecule of section (a) or (b) of this paragraph.

In particular, it was observed that in plants, especially in Arabidopsis thaliana, increasing or generating the activity of a gene product non-targeted with the activity of a “b4029-protein”, preferably being encoded by a gene comprising the nucleic acid sequence SEQ ID NO. 10740, preferably the coding region thereof, conferred the production of or the increase in gamma-tocopherol compared with the wild type control.

The nucleic acid sequence of B4050 from Escherichia coli, e.g. as shown in the respective line in column 5 of Table I, application no. 7, has been published in B. And the activity of the gene product thereof is the activity of b4050-protein.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “b4050-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said B4050, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B4050, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B4050, or a functional equivalent or a homolog thereof as depicted in column 8 of Table II, application no. 7, preferably a homolog or functional equivalent as depicted in column 8 of Table II B, application no. 7, and being depicted in the same respective line as said B4050, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “b4050-protein”, preferably it is the molecule of section (a) or (b) of this paragraph.

In particular, it was observed that in plants, especially in Arabidopsis thaliana, increasing or generating the activity of a gene product non-targeted with the activity of a “b4050-protein”, preferably being encoded by a gene comprising the nucleic acid sequence SEQ ID NO. 106532, preferably the coding region thereof, conferred the production of or the increase in alpha-tocopherol compared with the wild type control.

The nucleic acid sequence of B4056 from Escherichia coli, e.g. as shown in the respective line in column 5 of Table I, application no. 7, has been published in B. And the activity of the gene product thereof is the activity of b4056-protein.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “b4056-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said B4056, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B4056, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B4056, or a functional equivalent or a homolog thereof as depicted in column 8 of Table II, application no. 7, preferably a homolog or functional equivalent as depicted in column 8 of Table II B, application no. 7, and being depicted in the same respective line as said B4056, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “b4056-protein”, preferably it is the molecule of section (a) or (b) of this paragraph.

In particular, it was observed that in plants, especially in Arabidopsis thaliana, increasing or generating the activity of a gene product non-targeted with the activity of a “b4056-protein”, preferably being encoded by a gene comprising the nucleic acid sequence SEQ ID NO. 94542, preferably the coding region thereof, conferred the production of or the increase in alpha-tocopherol compared with the wild type control.

The nucleic acid sequence of B4256 from Escherichia coli, e.g. as shown in the respective line in column 5 of Table I, application no. 7, has been published in B. And the activity of the gene product thereof is the activity of acetyltransferase.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said B4256, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B4256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B4256, or a functional equivalent or a homolog thereof as depicted in column 8 of Table II, application no. 7, preferably a homolog or functional equivalent as depicted in column 8 of Table II B, application no. 7, and being depicted in the same respective line as said B4256, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 45321, preferably the coding region thereof, conferred the production of or the increase in alpha-tocopherol compared with the wild type control.

The nucleic acid sequence of B4256 from Escherichia coli, e.g. as shown in the respective line in column 5 of Table I, application no. 7, has been published in B. And the activity of the gene product thereof is the activity of acetyltransferase.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said B4256, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B4256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B4256, or a functional equivalent or a homolog thereof as depicted in column 8 of Table II, application no. 7, preferably a homolog or functional equivalent as depicted in column 8 of Table II B, application no. 7, and being depicted in the same respective line as said B4256, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 45321, preferably the coding region thereof, conferred the production of or the increase in gamma-tocopherol compared with the wild type control.

The nucleic acid sequence of B4321 from Escherichia coli, e.g. as shown in the respective line in column 5 of Table I, application no. 7, has been published in B. And the activity of the gene product thereof is the activity of gluconate transport system permease 3.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “gluconate transport system permease 3”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said B4321, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B4321, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B4321, or a functional equivalent or a homolog thereof as depicted in column 8 of Table II, application no. 7, preferably a homolog or functional equivalent as depicted in column 8 of Table II B, application no. 7, and being depicted in the same respective line as said B4321, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “gluconate transport system permease 3”, preferably it is the molecule of section (a) or (b) of this paragraph.

In particular, it was observed that in plants, especially in Arabidopsis thaliana, increasing or generating the activity of a gene product non-targeted with the activity of a “gluconate transport system permease 3”, preferably being encoded by a gene comprising the nucleic acid sequence SEQ ID NO. 45394, preferably the coding region thereof, conferred the production of or the increase in alpha-tocopherol compared with the wild type control.

The nucleic acid sequence of B4321 from Escherichia coli, e.g. as shown in the respective line in column 5 of Table I, application no. 7, has been published in B. And the activity of the gene product thereof is the activity of gluconate transport system permease 3.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “gluconate transport system permease 3”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said B4321, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said B4321, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said B4321, or a functional equivalent or a homolog thereof as depicted in column 8 of Table II, application no. 7, preferably a homolog or functional equivalent as depicted in column 8 of Table II B, application no. 7, and being depicted in the same respective line as said B4321, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “gluconate transport system permease 3”, preferably it is the molecule of section (a) or (b) of this paragraph.

In particular, it was observed that in plants, especially in Arabidopsis thaliana, increasing or generating the activity of a gene product non-targeted with the activity of a “gluconate transport system permease 3”, preferably being encoded by a gene comprising the nucleic acid sequence SEQ ID NO. 45394, preferably the coding region thereof, conferred the production of or the increase in gamma-tocopherol compared with the wild type control.

The nucleic acid sequence of C_pp004096192r from Physcomitrella patens, e.g. as shown in the respective line in column 5 of Table I, application no. 7, is unpublished. And the activity of the gene product thereof is the activity of thioredoxin.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “thioredoxin”, especially from Physcomitrella patens or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said C_pp004096192r, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said C_pp004096192r, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said C_pp004096192r, or a functional equivalent or a homolog thereof as depicted in column 8 of Table II, application no. 7, preferably a homolog or functional equivalent as depicted in column 8 of Table II B, application no. 7, and being depicted in the same respective line as said C_pp004096192r, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “thioredoxin”, preferably it is the molecule of section (a) or (b) of this paragraph.

In particular, it was observed that in plants, especially in Arabidopsis thaliana, increasing or generating the activity of a gene product non-targeted with the activity of a “thioredoxin”, preferably being encoded by a gene comprising the nucleic acid sequence SEQ ID NO. 45757, preferably the coding region thereof, conferred the production of or the increase in gamma-tocopherol compared with the wild type control.

The nucleic acid sequence of GM02LC12622 from Glycine max, e.g. as shown in the respective line in column 5 of Table I, application no. 7, is unpublished. And the activity of the gene product thereof is the activity of glutaredoxin.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “glutaredoxin”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said GM02LC12622, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said GM02LC12622, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said GM02LC12622, or a functional equivalent or a homolog thereof as depicted in column 8 of Table II, application no. 7, preferably a homolog or functional equivalent as depicted in column 8 of Table II B, application no. 7, and being depicted in the same respective line as said GM02LC12622, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 10811, preferably the coding region thereof, conferred the production of or the increase in alpha-tocopherol compared with the wild type control.

The nucleic acid sequence of GM02LC12622 from Glycine max, e.g. as shown in the respective line in column 5 of Table I, application no. 7, is unpublished. And the activity of the gene product thereof is the activity of glutaredoxin.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “glutaredoxin”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said GM02LC12622, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said GM02LC12622, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said GM02LC12622, or a functional equivalent or a homolog thereof as depicted in column 8 of Table II, application no. 7, preferably a homolog or functional equivalent as depicted in column 8 of Table II B, application no. 7, and being depicted in the same respective line as said GM02LC12622, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 10811, preferably the coding region thereof, conferred the production of or the increase in gamma-tocopherol compared with the wild type control.

The nucleic acid sequence of GM02LC21368 from Glycine max, e.g. as shown in the respective line in column 5 of Table I, application no. 7, is unpublished. And the activity of the gene product thereof is the activity of transcription factor.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said GM02LC21368, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said GM02LC21368, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said GM02LC21368, or a functional equivalent or a homolog thereof as depicted in column 8 of Table II, application no. 7, preferably a homolog or functional equivalent as depicted in column 8 of Table II B, application no. 7, and being depicted in the same respective line as said GM02LC21368, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 84198, preferably the coding region thereof, conferred the production of or the increase in gamma-tocopherol compared with the wild type control.

The nucleic acid sequence of Sll0290 from Synechocystis sp., e.g. as shown in the respective line in column 5 of Table I, application no. 7, has been published in C. And the activity of the gene product thereof is the activity of polyphosphate kinase.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “polyphosphate kinase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said Sll0290, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Sll0290, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Sll0290, or a functional equivalent or a homolog thereof as depicted in column 8 of Table II, application no. 7, preferably a homolog or functional equivalent as depicted in column 8 of Table II B, application no. 7, and being depicted in the same respective line as said Sll0290, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “polyphosphate kinase”, preferably it is the molecule of section (a) or (b) of this paragraph.

In particular, it was observed that in plants, especially in Arabidopsis thaliana, increasing or generating the activity of a gene product non-targeted with the activity of a “polyphosphate kinase”, preferably being encoded by a gene comprising the nucleic acid sequence SEQ ID NO. 49828, preferably the coding region thereof, conferred the production of or the increase in alpha-tocopherol compared with the wild type control.

The nucleic acid sequence of Sll0745 from Synechocystis sp., e.g. as shown in the respective line in column 5 of Table I, application no. 7, has been published in C. And the activity of the gene product thereof is the activity of phosphofructokinase.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “phosphofructokinase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said Sll0745, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Sll0745, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Sll0745, or a functional equivalent or a homolog thereof as depicted in column 8 of Table II, application no. 7, preferably a homolog or functional equivalent as depicted in column 8 of Table II B, application no. 7, and being depicted in the same respective line as said Sll0745, and preferably the activity is increased mitochondrial, whereby the respective line disclose in column 7 the fine chemical gamma-tocopherol.

Accordingly, in one embodiment, the molecule which activity is to be increased in the process of the invention is the gene product with an activity as a “phosphofructokinase”, preferably it is the molecule of section (a) or (b) of this paragraph.

In particular, it was observed that in plants, especially in Arabidopsis thaliana, increasing or generating the activity of a gene product mitochondrial with the activity of a “phosphofructokinase”, preferably being encoded by a gene comprising the nucleic acid sequence SEQ ID NO. 112661, preferably the coding region thereof, conferred the production of or the increase in gamma-tocopherol compared with the wild type control.

The nucleic acid sequence of Sll1185 from Synechocystis sp., e.g. as shown in the respective line in column 5 of Table I, application no. 7, has been published in C. And the activity of the gene product thereof is the activity of coproporphyrinogen oxidase.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “coproporphyrinogen oxidase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said Sll1185, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Sll1185, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Sll1185, or a functional equivalent or a homolog thereof as depicted in column 8 of Table II, application no. 7, preferably a homolog or functional equivalent as depicted in column 8 of Table II B, application no. 7, and being depicted in the same respective line as said Sll1185, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “coproporphyrinogen oxidase”, preferably it is the molecule of section (a) or (b) of this paragraph.

In particular, it was observed that in plants, especially in Arabidopsis thaliana, increasing or generating the activity of a gene product non-targeted with the activity of a “coproporphyrinogen oxidase”, preferably being encoded by a gene comprising the nucleic acid sequence SEQ ID NO. 53189, preferably the coding region thereof, conferred the production of or the increase in gamma-tocopherol compared with the wild type control.

The nucleic acid sequence of Sll1761 from Synechocystis sp., e.g. as shown in the respective line in column 5 of Table I, application no. 7, has been published in C. And the activity of the gene product thereof is the activity of sll1761-protein.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “sll1761-protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said Sll1761, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Sll1761, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Sll1761, or a functional equivalent or a homolog thereof as depicted in column 8 of Table II, application no. 7, preferably a homolog or functional equivalent as depicted in column 8 of Table II B, application no. 7, and being depicted in the same respective line as said Sll1761, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “sll1761-protein”, preferably it is the molecule of section (a) or (b) of this paragraph.

In particular, it was observed that in plants, especially in Arabidopsis thaliana, increasing or generating the activity of a gene product non-targeted with the activity of a “sll1761-protein”, preferably being encoded by a gene comprising the nucleic acid sequence SEQ ID NO. 55379, preferably the coding region thereof, conferred the production of or the increase in gamma-tocopherol compared with the wild type control.

The nucleic acid sequence of Sll1815 from Synechocystis sp., e.g. as shown in the respective line in column 5 of Table I, application no. 7, has been published in C. And the activity of the gene product thereof is the activity of adenylate kinase.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “adenylate kinase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said Sll1815, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Sll1815, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Sll1815, or a functional equivalent or a homolog thereof as depicted in column 8 of Table II, application no. 7, preferably a homolog or functional equivalent as depicted in column 8 of Table II B, application no. 7, and being depicted in the same respective line as said Sll1815, and preferably the activity is increased mitochondrial, whereby the respective line disclose in column 7 the fine chemical gamma-tocopherol.

Accordingly, in one embodiment, the molecule which activity is to be increased in the process of the invention is the gene product with an activity as a “adenylate kinase”, preferably it is the molecule of section (a) or (b) of this paragraph.

In particular, it was observed that in plants, especially in Arabidopsis thaliana, increasing or generating the activity of a gene product mitochondrial with the activity of a “adenylate kinase”, preferably being encoded by a gene comprising the nucleic acid sequence SEQ ID NO. 85808, preferably the coding region thereof, conferred the production of or the increase in gamma-tocopherol compared with the wild type control.

The nucleic acid sequence of Slr0239 from Synechocystis sp., e.g. as shown in the respective line in column 5 of Table I, application no. 7, has been published in C. And the activity of the gene product thereof is the activity of precorrin methylase.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “precorrin methylase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said Slr0239, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Slr0239, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Slr0239, or a functional equivalent or a homolog thereof as depicted in column 8 of Table II, application no. 7, preferably a homolog or functional equivalent as depicted in column 8 of Table II B, application no. 7, and being depicted in the same respective line as said Slr0239, and preferably the activity is increased mitochondrial, whereby the respective line disclose in column 7 the fine chemical alpha-tocopherol.

Accordingly, in one embodiment, the molecule which activity is to be increased in the process of the invention is the gene product with an activity as a “precorrin methylase”, preferably it is the molecule of section (a) or (b) of this paragraph.

In particular, it was observed that in plants, especially in Arabidopsis thaliana, increasing or generating the activity of a gene product mitochondrial with the activity of a “precorrin methylase”, preferably being encoded by a gene comprising the nucleic acid sequence SEQ ID NO. 86540, preferably the coding region thereof, conferred the production of or the increase in alpha-tocopherol compared with the wild type control.

The nucleic acid sequence of Slr0721 from Synechocystis sp., e.g. as shown in the respective line in column 5 of Table I, application no. 7, has been published in C. And the activity of the gene product thereof is the activity of malate dehydrogenase.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “malate dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said Slr0721, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Slr0721, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Slr0721, or a functional equivalent or a homolog thereof as depicted in column 8 of Table II, application no. 7, preferably a homolog or functional equivalent as depicted in column 8 of Table II B, application no. 7, and being depicted in the same respective line as said Slr0721, and preferably the activity is increased plastidic, whereby the respective line disclose in column 7 the fine chemical alpha-tocopherol.

Accordingly, in one embodiment, the molecule which activity is to be increased in the process of the invention is the gene product with an activity as a “malate dehydrogenase”, preferably it is the molecule of section (a) or (b) of this paragraph.

In particular, it was observed that in plants, especially in Arabidopsis thaliana, increasing or generating the activity of a gene product plastidic with the activity of a “malate dehydrogenase”, preferably being encoded by a gene comprising the nucleic acid sequence SEQ ID NO. 87397, preferably the coding region thereof, conferred the production of or the increase in alpha-tocopherol compared with the wild type control.

The nucleic acid sequence of Slr0721 from Synechocystis sp., e.g. as shown in the respective line in column 5 of Table I, application no. 7, has been published in C. And the activity of the gene product thereof is the activity of malate dehydrogenase.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “malate dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said Slr0721, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Slr0721, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Slr0721, or a functional equivalent or a homolog thereof as depicted in column 8 of Table II, application no. 7, preferably a homolog or functional equivalent as depicted in column 8 of Table II B, application no. 7, and being depicted in the same respective line as said Slr0721, and preferably the activity is increased plastidic, whereby the respective line disclose in column 7 the fine chemical gamma-tocopherol.

Accordingly, in one embodiment, the molecule which activity is to be increased in the process of the invention is the gene product with an activity as a “malate dehydrogenase”, preferably it is the molecule of section (a) or (b) of this paragraph.

In particular, it was observed that in plants, especially in Arabidopsis thaliana, increasing or generating the activity of a gene product plastidic with the activity of a “malate dehydrogenase”, preferably being encoded by a gene comprising the nucleic acid sequence SEQ ID NO. 87397, preferably the coding region thereof, conferred the production of or the increase in gamma-tocopherol compared with the wild type control.

The nucleic acid sequence of Slr0739 from Synechocystis sp., e.g. as shown in the respective line in column 5 of Table I, application no. 7, has been published in C. And the activity of the gene product thereof is the activity of geranylgeranyl pyrophosphate synthase.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “geranylgeranyl pyrophosphate synthase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said Slr0739, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Slr0739, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Slr0739, or a functional equivalent or a homolog thereof as depicted in column 8 of Table II, application no. 7, preferably a homolog or functional equivalent as depicted in column 8 of Table II B, application no. 7, and being depicted in the same respective line as said Slr0739, and preferably the activity is increased plastidic, whereby the respective line disclose in column 7 the fine chemical alpha-tocopherol.

Accordingly, in one embodiment, the molecule which activity is to be increased in the process of the invention is the gene product with an activity as a “geranylgeranyl pyrophosphate synthase”, preferably it is the molecule of section (a) or (b) of this paragraph.

In particular, it was observed that in plants, especially in Arabidopsis thaliana, increasing or generating the activity of a gene product plastidic with the activity of a “geranylgeranyl pyrophosphate synthase”, preferably being encoded by a gene comprising the nucleic acid sequence SEQ ID NO. 57235, preferably the coding region thereof, conferred the production of or the increase in alpha-tocopherol compared with the wild type control.

The nucleic acid sequence of Slr0739 from Synechocystis sp., e.g. as shown in the respective line in column 5 of Table I, application no. 7, has been published in C. And the activity of the gene product thereof is the activity of geranylgeranyl pyrophosphate synthase.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “geranylgeranyl pyrophosphate synthase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said Slr0739, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Slr0739, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Slr0739, or a functional equivalent or a homolog thereof as depicted in column 8 of Table II, application no. 7, preferably a homolog or functional equivalent as depicted in column 8 of Table II B, application no. 7, and being depicted in the same respective line as said Slr0739, and preferably the activity is increased plastidic, whereby the respective line disclose in column 7 the fine chemical gamma-tocopherol.

Accordingly, in one embodiment, the molecule which activity is to be increased in the process of the invention is the gene product with an activity as a “geranylgeranyl pyrophosphate synthase”, preferably it is the molecule of section (a) or (b) of this paragraph.

In particular, it was observed that in plants, especially in Arabidopsis thaliana, increasing or generating the activity of a gene product plastidic with the activity of a “geranylgeranyl pyrophosphate synthase”, preferably being encoded by a gene comprising the nucleic acid sequence SEQ ID NO. 57235, preferably the coding region thereof, conferred the production of or the increase in gamma-tocopherol compared with the wild type control.

The nucleic acid sequence of Slr1420 from Synechocystis sp., e.g. as shown in the respective line in column 5 of Table I, application no. 7, has been published in C. And the activity of the gene product thereof is the activity of carbohydrate kinase.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “carbohydrate kinase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said Slr1420, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Slr1420, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Slr1420, or a functional equivalent or a homolog thereof as depicted in column 8 of Table II, application no. 7, preferably a homolog or functional equivalent as depicted in column 8 of Table II B, application no. 7, and being depicted in the same respective line as said Slr1420, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “carbohydrate kinase”, preferably it is the molecule of section (a) or (b) of this paragraph.

In particular, it was observed that in plants, especially in Arabidopsis thaliana, increasing or generating the activity of a gene product non-targeted with the activity of a “carbohydrate kinase”, preferably being encoded by a gene comprising the nucleic acid sequence SEQ ID NO. 58590, preferably the coding region thereof, conferred the production of or the increase in gamma-tocopherol compared with the wild type control.

The nucleic acid sequence of Slr1739 from Synechocystis sp., e.g. as shown in the respective line in column 5 of Table I, application no. 7, has been published in C. And the activity of the gene product thereof is the activity of photosystem II protein.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “photosystem II protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said Slr1739, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Slr1739, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Slr1739, or a functional equivalent or a homolog thereof as depicted in column 8 of Table II, application no. 7, preferably a homolog or functional equivalent as depicted in column 8 of Table II B, application no. 7, and being depicted in the same respective line as said Slr1739, and preferably the activity is increased plastidic, whereby the respective line disclose in column 7 the fine chemical gamma-tocopherol.

Accordingly, in one embodiment, the molecule which activity is to be increased in the process of the invention is the gene product with an activity as a “photosystem II protein”, preferably it is the molecule of section (a) or (b) of this paragraph.

In particular, it was observed that in plants, especially in Arabidopsis thaliana, increasing or generating the activity of a gene product plastidic with the activity of a “photosystem II protein”, preferably being encoded by a gene comprising the nucleic acid sequence SEQ ID NO. 58731, preferably the coding region thereof, conferred the production of or the increase in gamma-tocopherol compared with the wild type control.

The nucleic acid sequence of Slr1755 from Synechocystis sp., e.g. as shown in the respective line in column 5 of Table I, application no. 7, has been published in C. And the activity of the gene product thereof is the activity of glycerol-3-phosphate dehydrogenase.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “glycerol-3-phosphate dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said Slr1755, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Slr1755, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Slr1755, or a functional equivalent or a homolog thereof as depicted in column 8 of Table II, application no. 7, preferably a homolog or functional equivalent as depicted in column 8 of Table II B, application no. 7, and being depicted in the same respective line as said Slr1755, and preferably the activity is increased plastidic, whereby the respective line disclose in column 7 the fine chemical alpha-tocopherol.

Accordingly, in one embodiment, the molecule which activity is to be increased in the process of the invention is the gene product with an activity as a “glycerol-3-phosphate dehydrogenase”, preferably it is the molecule of section (a) or (b) of this paragraph.

In particular, it was observed that in plants, especially in Arabidopsis thaliana, increasing or generating the activity of a gene product plastidic with the activity of a “glycerol-3-phosphate dehydrogenase”, preferably being encoded by a gene comprising the nucleic acid sequence SEQ ID NO. 58823, preferably the coding region thereof, conferred the production of or the increase in alpha-tocopherol compared with the wild type control.

The nucleic acid sequence of Slr1791 from Synechocystis sp., e.g. as shown in the respective line in column 5 of Table I, application no. 7, has been published in C. And the activity of the gene product thereof is the activity of phosphoadenosine phosphosulfate reductase .

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “phosphoadenosine phosphosulfate reductase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said Slr1791, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Slr1791, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Slr1791, or a functional equivalent or a homolog thereof as depicted in column 8 of Table II, application no. 7, preferably a homolog or functional equivalent as depicted in column 8 of Table II B, application no. 7, and being depicted in the same respective line as said Slr1791, and preferably the activity is increased plastidic, whereby the respective line disclose in column 7 the fine chemical gamma-tocopherol.

Accordingly, in one embodiment, the molecule which activity is to be increased in the process of the invention is the gene product with an activity as a “phosphoadenosine phosphosulfate reductase”, preferably it is the molecule of section (a) or (b) of this paragraph.

In particular, it was observed that in plants, especially in Arabidopsis thaliana, increasing or generating the activity of a gene product plastidic with the activity of a “phosphoadenosine phosphosulfate reductase”, preferably being encoded by a gene comprising the nucleic acid sequence SEQ ID NO. 12140, preferably the coding region thereof, conferred the production of or the increase in gamma-tocopherol compared with the wild type control.

The nucleic acid sequence of Slr1917 from Synechocystis sp., e.g. as shown in the respective line in column 5 of Table I, application no. 7, has been published in C. And the activity of the gene product thereof is the activity of slr1917-protein.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “slr1917-protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said Slr1917, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Slr1917, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Slr1917, or a functional equivalent or a homolog thereof as depicted in column 8 of Table II, application no. 7, preferably a homolog or functional equivalent as depicted in column 8 of Table II B, application no. 7, and being depicted in the same respective line as said Slr1917, and preferably the activity is increased plastidic, whereby the respective line disclose in column 7 the fine chemical alpha-tocopherol.

Accordingly, in one embodiment, the molecule which activity is to be increased in the process of the invention is the gene product with an activity as a “slr1917-protein”, preferably it is the molecule of section (a) or (b) of this paragraph.

In particular, it was observed that in plants, especially in Arabidopsis thaliana, increasing or generating the activity of a gene product plastidic with the activity of a “slr1917-protein”, preferably being encoded by a gene comprising the nucleic acid sequence SEQ ID NO. 113038, preferably the coding region thereof, conferred the production of or the increase in alpha-tocopherol compared with the wild type control.

The nucleic acid sequence of Slr1917 from Synechocystis sp., e.g. as shown in the respective line in column 5 of Table I, application no. 7, has been published in C. And the activity of the gene product thereof is the activity of slr1917-protein.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “slr1917-protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said Slr1917, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Slr1917, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Slr1917, or a functional equivalent or a homolog thereof as depicted in column 8 of Table II, application no. 7, preferably a homolog or functional equivalent as depicted in column 8 of Table II B, application no. 7, and being depicted in the same respective line as said Slr1917, and preferably the activity is increased plastidic, whereby the respective line disclose in column 7 the fine chemical gamma-tocopherol.

Accordingly, in one embodiment, the molecule which activity is to be increased in the process of the invention is the gene product with an activity as a “slr1917-protein”, preferably it is the molecule of section (a) or (b) of this paragraph.

In particular, it was observed that in plants, especially in Arabidopsis thaliana, increasing or generating the activity of a gene product plastidic with the activity of a “slr1917-protein”, preferably being encoded by a gene comprising the nucleic acid sequence SEQ ID NO. 113038, preferably the coding region thereof, conferred the production of or the increase in gamma-tocopherol compared with the wild type control.

The nucleic acid sequence of TTC0019 from Thermus thermophilus, e.g. as shown in the respective line in column 5 of Table I, application no. 7, has been published in E. And the activity of the gene product thereof is the activity of Sec-independent protein translocase subunit.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “Sec-independent protein translocase subunit”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said TTC0019, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said TTC0019, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said TTC0019, or a functional equivalent or a homolog thereof as depicted in column 8 of Table II, application no. 7, preferably a homolog or functional equivalent as depicted in column 8 of Table II B, application no. 7, and being depicted in the same respective line as said TTC0019, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “Sec-independent protein translocase subunit”, preferably it is the molecule of section (a) or (b) of this paragraph.

In particular, it was observed that in plants, especially in Arabidopsis thaliana, increasing or generating the activity of a gene product non-targeted with the activity of a “Sec-independent protein translocase subunit”, preferably being encoded by a gene comprising the nucleic acid sequence SEQ ID NO. 12698, preferably the coding region thereof, conferred the production of or the increase in alpha-tocopherol compared with the wild type control.

The nucleic acid sequence of TTC0019 from Thermus thermophilus, e.g. as shown in the respective line in column 5 of Table I, application no. 7, has been published in E. And the activity of the gene product thereof is the activity of Sec-independent protein translocase subunit.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “Sec-independent protein translocase subunit”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said TTC0019, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said TTC0019, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said TTC0019, or a functional equivalent or a homolog thereof as depicted in column 8 of Table II, application no. 7, preferably a homolog or functional equivalent as depicted in column 8 of Table II B, application no. 7, and being depicted in the same respective line as said TTC0019, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “Sec-independent protein translocase subunit”, preferably it is the molecule of section (a) or (b) of this paragraph.

In particular, it was observed that in plants, especially in Arabidopsis thaliana, increasing or generating the activity of a gene product non-targeted with the activity of a “Sec-independent protein translocase subunit”, preferably being encoded by a gene comprising the nucleic acid sequence SEQ ID NO. 12698, preferably the coding region thereof, conferred the production of or the increase in gamma-tocopherol compared with the wild type control.

The nucleic acid sequence of TTC1193 from Thermus thermophilus, e.g. as shown in the respective line in column 5 of Table I, application no. 7, has been published in E. And the activity of the gene product thereof is the activity of multiple antibiotic resistance protein.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “multiple antibiotic resistance protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said TTC1193, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said TTC1193, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said TTC1193, or a functional equivalent or a homolog thereof as depicted in column 8 of Table II, application no. 7, preferably a homolog or functional equivalent as depicted in column 8 of Table II B, application no. 7, and being depicted in the same respective line as said TTC1193, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “multiple antibiotic resistance protein”, preferably it is the molecule of section (a) or (b) of this paragraph.

In particular, it was observed that in plants, especially in Arabidopsis thaliana, increasing or generating the activity of a gene product non-targeted with the activity of a “multiple antibiotic resistance protein”, preferably being encoded by a gene comprising the nucleic acid sequence SEQ ID NO. 61723, preferably the coding region thereof, conferred the production of or the increase in gamma-tocopherol compared with the wild type control.

The nucleic acid sequence of TTC1386 from Thermus thermophilus, e.g. as shown in the respective line in column 5 of Table I, application no. 7, has been published in E. And the activity of the gene product thereof is the activity of TTC1386-protein.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “TTC1386-protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said TTC1386, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said TTC1386, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said TTC1386, or a functional equivalent or a homolog thereof as depicted in column 8 of Table II, application no. 7, preferably a homolog or functional equivalent as depicted in column 8 of Table II B, application no. 7, and being depicted in the same respective line as said TTC1386, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “TTC1386-protein”, preferably it is the molecule of section (a) or (b) of this paragraph.

In particular, it was observed that in plants, especially in Arabidopsis thaliana, increasing or generating the activity of a gene product non-targeted with the activity of a “TTC1386-protein”, preferably being encoded by a gene comprising the nucleic acid sequence SEQ ID NO. 62079, preferably the coding region thereof, conferred the production of or the increase in alpha-tocopherol compared with the wild type control.

The nucleic acid sequence of TTC1386 from Thermus thermophilus, e.g. as shown in the respective line in column 5 of Table I, application no. 7, has been published in E. And the activity of the gene product thereof is the activity of TTC1386-protein.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “TTC1386-protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said TTC1386, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said TTC1386, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said TTC1386, or a functional equivalent or a homolog thereof as depicted in column 8 of Table II, application no. 7, preferably a homolog or functional equivalent as depicted in column 8 of Table II B, application no. 7, and being depicted in the same respective line as said TTC1386, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “TTC1386-protein”, preferably it is the molecule of section (a) or (b) of this paragraph.

In particular, it was observed that in plants, especially in Arabidopsis thaliana, increasing or generating the activity of a gene product non-targeted with the activity of a “TTC1386-protein”, preferably being encoded by a gene comprising the nucleic acid sequence SEQ ID NO. 62079, preferably the coding region thereof, conferred the production of or the increase in gamma-tocopherol compared with the wild type control.

The nucleic acid sequence of TTC1550 from Thermus thermophilus, e.g. as shown in the respective line in column 5 of Table I, application no. 7, has been published in E. And the activity of the gene product thereof is the activity of homocitrate synthase.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “homocitrate synthase”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said TTC1550, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said TTC1550, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said TTC1550, or a functional equivalent or a homolog thereof as depicted in column 8 of Table II, application no. 7, preferably a homolog or functional equivalent as depicted in column 8 of Table II B, application no. 7, and being depicted in the same respective line as said TTC1550, and preferably the activity is increased non-targeted, whereby the respective line disclose in column 7 the fine chemical alpha-tocopherol.

Accordingly, in one embodiment, the molecule which activity is to be increased in the process of the invention is the gene product with an activity as a “homocitrate synthase”, preferably it is the molecule of section (a) or (b) of this paragraph.

In particular, it was observed that in plants, especially in Arabidopsis thaliana, increasing or generating the activity of a gene product non-targeted with the activity of a “homocitrate synthase”, preferably being encoded by a gene comprising the nucleic acid sequence SEQ ID NO. 12974, preferably the coding region thereof, conferred the production of or the increase in alpha-tocopherol compared with the wild type control.

The nucleic acid sequence of Ybr249c from Saccharomyces cerevisiae, e.g. as shown in the respective line in column 5 of Table I, application no. 7, has been published in A. And the activity of the gene product thereof is the activity of 3-deoxy-7-phosphoheptulonate synthase.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “3-deoxy-7-phosphoheptulonate synthase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said Ybr249c, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Ybr249c, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Ybr249c, or a functional equivalent or a homolog thereof as depicted in column 8 of Table II, application no. 7, preferably a homolog or functional equivalent as depicted in column 8 of Table II B, application no. 7, and being depicted in the same respective line as said Ybr249c, and preferably the activity is increased plastidic, whereby the respective line disclose in column 7 the fine chemical alpha-tocopherol.

Accordingly, in one embodiment, the molecule which activity is to be increased in the process of the invention is the gene product with an activity as a “3-deoxy-7-phosphoheptulonate synthase”, preferably it is the molecule of section (a) or (b) of this paragraph.

In particular, it was observed that in plants, especially in Arabidopsis thaliana, increasing or generating the activity of a gene product plastidic with the activity of a “3-deoxy-7-phosphoheptulonate synthase”, preferably being encoded by a gene comprising the nucleic acid sequence SEQ ID NO. 88879, preferably the coding region thereof, conferred the production of or the increase in alpha-tocopherol compared with the wild type control.

The nucleic acid sequence of Ybr249c from Saccharomyces cerevisiae, e.g. as shown in the respective line in column 5 of Table I, application no. 7, has been published in A. And the activity of the gene product thereof is the activity of 3-deoxy-7-phosphoheptulonate synthase.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “3-deoxy-7-phosphoheptulonate synthase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said Ybr249c, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Ybr249c, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Ybr249c, or a functional equivalent or a homolog thereof as depicted in column 8 of Table II, application no. 7, preferably a homolog or functional equivalent as depicted in column 8 of Table II B, application no. 7, and being depicted in the same respective line as said Ybr249c, and preferably the activity is increased plastidic, whereby the respective line disclose in column 7 the fine chemical gamma-tocopherol.

Accordingly, in one embodiment, the molecule which activity is to be increased in the process of the invention is the gene product with an activity as a “3-deoxy-7-phosphoheptulonate synthase”, preferably it is the molecule of section (a) or (b) of this paragraph.

In particular, it was observed that in plants, especially in Arabidopsis thaliana, increasing or generating the activity of a gene product plastidic with the activity of a “3-deoxy-7-phosphoheptulonate synthase”, preferably being encoded by a gene comprising the nucleic acid sequence SEQ ID NO. 88879, preferably the coding region thereof, conferred the production of or the increase in gamma-tocopherol compared with the wild type control.

The nucleic acid sequence of Ycl049c from Saccharomyces cerevisiae, e.g. as shown in the respective line in column 5 of Table I, application no. 7, has been published in A. And the activity of the gene product thereof is the activity of ycl049c-protein.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “ycl049c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said Ycl049c, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Ycl049c, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Ycl049c, or a functional equivalent or a homolog thereof as depicted in column 8 of Table II, application no. 7, preferably a homolog or functional equivalent as depicted in column 8 of Table II B, application no. 7, and being depicted in the same respective line as said Ycl049c, and preferably the activity is increased plastidic, whereby the respective line disclose in column 7 the fine chemical gamma-tocopherol.

Accordingly, in one embodiment, the molecule which activity is to be increased in the process of the invention is the gene product with an activity as a “ycl049c-protein”, preferably it is the molecule of section (a) or (b) of this paragraph.

In particular, it was observed that in plants, especially in Arabidopsis thaliana, increasing or generating the activity of a gene product plastidic with the activity of a “ycl049c-protein”, preferably being encoded by a gene comprising the nucleic acid sequence SEQ ID NO. 113220, preferably the coding region thereof, conferred the production of or the increase in gamma-tocopherol compared with the wild type control.

The nucleic acid sequence of Ydl131w from Saccharomyces cerevisiae, e.g. as shown in the respective line in column 5 of Table I, application no. 7, has been published in A. And the activity of the gene product thereof is the activity of homocitrate synthase.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “homocitrate synthase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said Ydl131w, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Ydl131w, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Ydl131w, or a functional equivalent or a homolog thereof as depicted in column 8 of Table II, application no. 7, preferably a homolog or functional equivalent as depicted in column 8 of Table II B, application no. 7, and being depicted in the same respective line as said Ydl131w, and preferably the activity is increased plastidic, whereby the respective line disclose in column 7 the fine chemical alpha-tocopherol.

Accordingly, in one embodiment, the molecule which activity is to be increased in the process of the invention is the gene product with an activity as a “homocitrate synthase”, preferably it is the molecule of section (a) or (b) of this paragraph.

In particular, it was observed that in plants, especially in Arabidopsis thaliana, increasing or generating the activity of a gene product plastidic with the activity of a “homocitrate synthase”, preferably being encoded by a gene comprising the nucleic acid sequence SEQ ID NO. 113224, preferably the coding region thereof, conferred the production of or the increase in alpha-tocopherol compared with the wild type control.

The nucleic acid sequence of Ydl235c from Saccharomyces cerevisiae, e.g. as shown in the respective line in column 5 of Table I, application no. 7, has been published in A. And the activity of the gene product thereof is the activity of ydl235c-protein.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “ydl235c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said Ydl235c, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Ydl235c, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Ydl235c, or a functional equivalent or a homolog thereof as depicted in column 8 of Table II, application no. 7, preferably a homolog or functional equivalent as depicted in column 8 of Table II B, application no. 7, and being depicted in the same respective line as said Ydl235c, and preferably the activity is increased plastidic, whereby the respective line disclose in column 7 the fine chemical alpha-tocopherol.

Accordingly, in one embodiment, the molecule which activity is to be increased in the process of the invention is the gene product with an activity as a “ydl235c-protein”, preferably it is the molecule of section (a) or (b) of this paragraph.

In particular, it was observed that in plants, especially in Arabidopsis thaliana, increasing or generating the activity of a gene product plastidic with the activity of a “ydl235c-protein”, preferably being encoded by a gene comprising the nucleic acid sequence SEQ ID NO. 113313, preferably the coding region thereof, conferred the production of or the increase in alpha-tocopherol compared with the wild type control.

The nucleic acid sequence of Ydr007w from Saccharomyces cerevisiae, e.g. as shown in the respective line in column 5 of Table I, application no. 7, has been published in A. And the activity of the gene product thereof is the activity of N-(5′-phosphoribosyl)anthranilate isomerase.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “N-(5′-phosphoribosyl)anthranilate isomerase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said Ydr007w, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Ydr007w, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Ydr007w, or a functional equivalent or a homolog thereof as depicted in column 8 of Table II, application no. 7, preferably a homolog or functional equivalent as depicted in column 8 of Table II B, application no. 7, and being depicted in the same respective line as said Ydr007w, and preferably the activity is increased plastidic, whereby the respective line disclose in column 7 the fine chemical gamma-tocopherol.

Accordingly, in one embodiment, the molecule which activity is to be increased in the process of the invention is the gene product with an activity as a “N-(5′-phosphoribosyl)anthranilate isomerase”, preferably it is the molecule of section (a) or (b) of this paragraph.

In particular, it was observed that in plants, especially in Arabidopsis thaliana, increasing or generating the activity of a gene product plastidic with the activity of a “N-(5′-phosphoribosyl) anthranilate isomerase”, preferably being encoded by a gene comprising the nucleic acid sequence SEQ ID NO. 89973, preferably the coding region thereof, conferred the production of or the increase in gamma-tocopherol compared with the wild type control.

The nucleic acid sequence of Ydr035w from Saccharomyces cerevisiae, e.g. as shown in the respective line in column 5 of Table I, application no. 7, has been published in A. And the activity of the gene product thereof is the activity of phospho-2-dehydro-3-deoxyheptonate aldolase.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “phospho-2-dehydro-3-deoxyheptonate aldolase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said Ydr035w, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Ydr035w, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Ydr035w, or a functional equivalent or a homolog thereof as depicted in column 8 of Table II, application no. 7, preferably a homolog or functional equivalent as depicted in column 8 of Table II B, application no. 7, and being depicted in the same respective line as said Ydr035w, and preferably the activity is increased plastidic, whereby the respective line disclose in column 7 the fine chemical gamma-tocopherol.

Accordingly, in one embodiment, the molecule which activity is to be increased in the process of the invention is the gene product with an activity as a “phospho-2-dehydro-3-deoxyheptonate aldolase”, preferably it is the molecule of section (a) or (b) of this paragraph.

In particular, it was observed that in plants, especially in Arabidopsis thaliana, increasing or generating the activity of a gene product plastidic with the activity of a “phospho-2-dehydro-3-deoxyheptonate aldolase”, preferably being encoded by a gene comprising the nucleic acid sequence SEQ ID NO. 113330, preferably the coding region thereof, conferred the production of or the increase in gamma-tocopherol compared with the wild type control.

The nucleic acid sequence of Ydr183w from Saccharomyces cerevisiae, e.g. as shown in the respective line in column 5 of Table I, application no. 7, has been published in A. And the activity of the gene product thereof is the activity of ydr183w-protein.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “ydr183w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said Ydr183w, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Ydr183w, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Ydr183w, or a functional equivalent or a homolog thereof as depicted in column 8 of Table II, application no. 7, preferably a homolog or functional equivalent as depicted in column 8 of Table II B, application no. 7, and being depicted in the same respective line as said Ydr183w, and preferably the activity is increased plastidic, whereby the respective line disclose in column 7 the fine chemical gamma-tocopherol.

Accordingly, in one embodiment, the molecule which activity is to be increased in the process of the invention is the gene product with an activity as a “ydr183w-protein”, preferably it is the molecule of section (a) or (b) of this paragraph.

In particular, it was observed that in plants, especially in Arabidopsis thaliana, increasing or generating the activity of a gene product plastidic with the activity of a “ydr183w-protein”, preferably being encoded by a gene comprising the nucleic acid sequence SEQ ID NO. 90103, preferably the coding region thereof, conferred the production of or the increase in gamma-tocopherol compared with the wild type control.

The nucleic acid sequence of Yer106w from Saccharomyces cerevisiae, e.g. as shown in the respective line in column 5 of Table I, application no. 7, has been published in A. And the activity of the gene product thereof is the activity of yer106w-protein.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “yer106w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said Yer106w, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Yer106w, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Yer106w, or a functional equivalent or a homolog thereof as depicted in column 8 of Table II, application no. 7, preferably a homolog or functional equivalent as depicted in column 8 of Table II B, application no. 7, and being depicted in the same respective line as said Yer106w, and preferably the activity is increased plastidic, whereby the respective line disclose in column 7 the fine chemical gamma-tocopherol.

Accordingly, in one embodiment, the molecule which activity is to be increased in the process of the invention is the gene product with an activity as a “yer106w-protein”, preferably it is the molecule of section (a) or (b) of this paragraph.

In particular, it was observed that in plants, especially in Arabidopsis thaliana, increasing or generating the activity of a gene product plastidic with the activity of a “yer106w-protein”, preferably being encoded by a gene comprising the nucleic acid sequence SEQ ID NO. 63803, preferably the coding region thereof, conferred the production of or the increase in gamma-tocopherol compared with the wild type control.

The nucleic acid sequence of Yer112w from Saccharomyces cerevisiae, e.g. as shown in the respective line in column 5 of Table I, application no. 7, has been published in A. And the activity of the gene product thereof is the activity of small nuclear ribonucleoprotein .

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “small nuclear ribonucleoprotein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said Yer112w, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Yer112w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Yer112w, or a functional equivalent or a homolog thereof as depicted in column 8 of Table II, application no. 7, preferably a homolog or functional equivalent as depicted in column 8 of Table II B, application no. 7, and being depicted in the same respective line as said Yer112w, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “small nuclear ribonucleoprotein”, preferably it is the molecule of section (a) or (b) of this paragraph.

In particular, it was observed that in plants, especially in Arabidopsis thaliana, increasing or generating the activity of a gene product non-targeted with the activity of a “small nuclear ribonucleoprotein”, preferably being encoded by a gene comprising the nucleic acid sequence SEQ ID NO. 113598, preferably the coding region thereof, conferred the production of or the increase in gamma-tocopherol compared with the wild type control.

The nucleic acid sequence of Ygr170w from Saccharomyces cerevisiae, e.g. as shown in the respective line in column 5 of Table I, application no. 7, has been published in A. And the activity of the gene product thereof is the activity of phosphatidylserine decarboxylase.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “phosphatidylserine decarboxylase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said Ygr170w, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Ygr170w, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Ygr170w, or a functional equivalent or a homolog thereof as depicted in column 8 of Table II, application no. 7, preferably a homolog or functional equivalent as depicted in column 8 of Table II B, application no. 7, and being depicted in the same respective line as said Ygr170w, and preferably the activity is increased plastidic, whereby the respective line disclose in column 7 the fine chemical alpha-tocopherol.

Accordingly, in one embodiment, the molecule which activity is to be increased in the process of the invention is the gene product with an activity as a “phosphatidylserine decarboxylase”, preferably it is the molecule of section (a) or (b) of this paragraph.

In particular, it was observed that in plants, especially in Arabidopsis thaliana, increasing or generating the activity of a gene product plastidic with the activity of a “phosphatidylserine decarboxylase”, preferably being encoded by a gene comprising the nucleic acid sequence SEQ ID NO. 113636, preferably the coding region thereof, conferred the production of or the increase in alpha-tocopherol compared with the wild type control.

The nucleic acid sequence of Yjr153w from Saccharomyces cerevisiae, e.g. as shown in the respective line in column 5 of Table I, application no. 7, has been published in A. And the activity of the gene product thereof is the activity of polygalacturonase.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “polygalacturonase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said Yjr153w, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Yjr153w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Yjr153w, or a functional equivalent or a homolog thereof as depicted in column 8 of Table II, application no. 7, preferably a homolog or functional equivalent as depicted in column 8 of Table II B, application no. 7, and being depicted in the same respective line as said Yjr153w, and preferably the activity is increased non-targeted, whereby the respective line disclose in column 7 the fine chemical gamma-tocopherol.

Accordingly, in one embodiment, the molecule which activity is to be increased in the process of the invention is the gene product with an activity as a “polygalacturonase”, preferably it is the molecule of section (a) or (b) of this paragraph.

In particular, it was observed that in plants, especially in Arabidopsis thaliana, increasing or generating the activity of a gene product non-targeted with the activity of a “polygalacturonase”, preferably being encoded by a gene comprising the nucleic acid sequence SEQ ID NO. 66274, preferably the coding region thereof, conferred the production of or the increase in gamma-tocopherol compared with the wild type control.

The nucleic acid sequence of Ylr178c from Saccharomyces cerevisiae, e.g. as shown in the respective line in column 5 of Table I, application no. 7, has been published in A. And the activity of the gene product thereof is the activity of ylr178c-protein.

Accordingly, in one embodiment, the process of the present invention for producing gamma-tocopherol in a non-human organism, like a microorganism or a plant or a part thereof, comprises increasing or generating the activity of a gene product with the activity of a gene product conferring the activity of “yid 78c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical gamma-tocopherol), application no. 7, preferably the coding region thereof, or a homolog or a fragment thereof, and being depicted in the same respective line as said Ylr178c, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Ylr178c, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Ylr178c, or a functional equivalent or a homolog thereof as depicted in column 8 of Table II, application no. 7, preferably a homolog or functional equivalent as depicted in column 8 of Table II B, application no. 7, and being depicted in the same respective line as said Ylr178c, and preferably the activity is increased plastidic, whereby the respective line disclose in column 7 the fine chemical gamma-tocopherol.

Accordingly, in one embodiment, the molecule which activity is to be increased in the process of the invention is the gene product with an activity as a “ylr178c-protein”, preferably it is the molecule of section (a) or (b) of this paragraph.

In particular, it was observed that in plants, especially in Arabidopsis thaliana, increasing or generating the activity of a gene product plastidic with the activity of a “yid 78c-protein”, preferably being encoded by a gene comprising the nucleic acid sequence SEQ ID NO. 66715, preferably the coding region thereof, conferred the production of or the increase in gamma-tocopherol compared with the wild type control.

The nucleic acid sequence of Ylr359w from Saccharomyces cerevisiae, e.g. as shown in the respective line in column 5 of Table I, application no. 7, has been published in A. And the activity of the gene product thereof is the activity of adenylosuccinate lyase.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “adenylosuccinate lyase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said Ylr359w, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Ylr359w, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Ylr359w, or a functional equivalent or a homolog thereof as depicted in column 8 of Table II, application no. 7, preferably a homolog or functional equivalent as depicted in column 8 of Table II B, application no. 7, and being depicted in the same respective line as said Ylr359w, and preferably the activity is increased plastidic, whereby the respective line disclose in column 7 the fine chemical alpha-tocopherol.

Accordingly, in one embodiment, the molecule which activity is to be increased in the process of the invention is the gene product with an activity as a “adenylosuccinate lyase”, preferably it is the molecule of section (a) or (b) of this paragraph.

In particular, it was observed that in plants, especially in Arabidopsis thaliana, increasing or generating the activity of a gene product plastidic with the activity of a “adenylosuccinate lyase”, preferably being encoded by a gene comprising the nucleic acid sequence SEQ ID NO. 67190, preferably the coding region thereof, conferred the production of or the increase in alpha-tocopherol compared with the wild type control.

The nucleic acid sequence of Ypr170c from Saccharomyces cerevisiae, e.g. as shown in the respective line in column 5 of Table I, application no. 7, has been published in A. And the activity of the gene product thereof is the activity of ypr170c-protein.

Accordingly, in one embodiment, the process of the present invention for producing alpha-tocopherol in a non-human organism, like a microorganism or a plant or a part thereof, comprises increasing or generating the activity of a gene product with the activity of a gene product conferring the activity of “ypr170c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical alpha-tocopherol), application no. 7, preferably the coding region thereof, or a homolog or a fragment thereof, and being depicted in the same respective line as said Ypr170c, or a functional equivalent or a homolog thereof as shown in column 8 of Table I, application no. 7, preferably the coding region thereof, particularly a homolog or functional equivalent as shown in column 8 of Table I B, application no. 7, and being depicted in the same respective line as said Ypr170c, and preferably the activity is increased plastidic, or
(b) a polypeptide comprising a polypeptide, a consensus sequence or at least a polypeptide motif as shown in the respective line in column 5 of Table II or in column 8 of Table IV, application no. 7, respectively, and being depicted in the same respective line as said Ypr170c, or a functional equivalent or a homolog thereof as depicted in column 8 of Table II, application no. 7, preferably a homolog or functional equivalent as depicted in column 8 of Table II B, application no. 7, and being depicted in the same respective line as said Ypr170c, and preferably the activity is increased plastidic, whereby the respective line disclose in column 7 the fine chemical alpha-tocopherol.

Accordingly, in one embodiment, the molecule which activity is to be increased in the process of the invention is the gene product with an activity as a “ypr170c-protein”, preferably it is the molecule of section (a) or (b) of this paragraph.

In particular, it was observed that in plants, especially in Arabidopsis thaliana, increasing or generating the activity of a gene product plastidic with the activity of a “ypr170c-protein”, preferably being encoded by a gene comprising the nucleic acid sequence SEQ ID NO. 113647, preferably the coding region thereof, conferred the production of or the increase in alpha-tocopherol compared with the wild type control.

It was further observed that increasing or generating the activity of a FCRP gene shown in Table d, e.g. a nucleic acid molecule derived from the coding region of the nucleic acid molecule shown in Table d in A. thaliana conferred the production of or the increase in fine chemical, compared with the wild type control. Thus, in one embodiment, a nucleic acid molecule, preferably the coding region thereof, indicated in Table d or its homolog as indicated in the respective line in Table I or the expression product is used in the method of the present invention to generate or to increase the production of fine chemical in a non-human organism, like a microorganism or a plant, compared with the wild type control.

In this context, the fine chemical amount in a cell, in a non-human organism, like a plant or a microorganism or a part thereof, is increased by 1%, 3%, 5%, 10%, 20%, 30%, 40%, 50%, 60%, 70%, 80%, 90%, 100%, 120, 150% or 200% or more.

The fine chemical can be contained in the organism either in its free form and/or bound to proteins or polypeptides or mixtures thereof. Accordingly, in one embodiment, the amount of the free form in a cell, in a non-human organism, like a plant or a microorganism or part thereof, is increased byl%, 3%, 5%, 10%, 20%, 30%, 40%, 50%, 60%, 70%, 80%, 90%, 100%, 120, 150% or 200% or more. Accordingly, in another embodiment, the amount of the bound fine chemical in a cell, in a non-human organism, like a plant or a microorganism or part thereof, is increased by 1%, 3%, 5%, 10%, 20%, 30%, 40%, 50%, 60%, 70%, 80%, 90%, 100%, 120, 150% or 200% or more.

A protein having an activity conferring an increase in the amount or level of the fine chemical alpha-tocopherol, or gamma-tocopherol, upon targeting to the plastids or mitochondria or upon non-targeting, preferably has the structure of the respective polypeptide described herein, in particular of the polypeptides comprising the consensus sequence or at least one polypeptide motifs as shown in the respective line in Table IV, application no. 7, column 8 or of the polypeptide comprising an amino acid sequence as disclosed in the respective line in Table II, application no. 7, columns 5 or 8, or homologs or fragments thereof as described herein, or is encoded by the nucleic acid molecule characterized herein or the nucleic acid molecule according to the invention, for example by the nucleic acid molecule as shown in the respective line in Table I, application no. 7, columns 5 or 8, preferably the coding region thereof, or homologs or fragments thereof and has the herein mentioned activity.

Owing to the biological activity of the proteins which are used in the process according to the invention and which are encoded by nucleic acid molecules according to the invention, it is possible to produce compositions comprising the fine chemical, i.e. an increased amount of the fine chemical free or bound, e.g. amino acid compositions. Depending on the choice of the non-human organism used for the process according to the present invention, for example a microorganism or a plant, compositions or mixtures comprising the fine chemical can be produced.

The term “expression” refers to the transcription and/or translation of a codogenic gene segment or gene. As a rule, the resulting product is an mRNA or a protein. However, expression products can also include functional RNAs such as, for example, antisense, nucleic acids, tRNAs, snRNAs, rRNAs, RNAi, siRNA, ribozymes etc. Expression may be systemic, local or temporal, for example limited to certain cell types, tissues organs or organelles or time periods.

In one embodiment, the process of the present invention comprises one or more of the following steps
(a) stabilizing a protein conferring the generation or increased expression of a protein encoded by the nucleic acid molecule of the invention or of the polypeptide of the invention, e.g. of a polypeptide having the activity selected from the group consisting of 3-deoxy-7-phosphoheptulonate synthase, 3-deoxy-D-arabinoheptulosonate-7-phosphatesynthase, ABC transporter permease subunit, acetyltransferase, acid shock protein, adenylate kinase, adenylosuccinate lyase, amino-acid acetyltransferase, ankyrin repeat family protein, At1g29350-protein, ATP-binding component of a transport system, b1163-protein, b1219-protein, b1234-protein, b1522-protein, b1921-protein, b2360-protein, b2548-protein, b2613-protein, b2673-protein, b2812-protein, b3890-protein, b3989-protein, b4029-protein, b4050-protein, b4056-protein, calcium-dependent protein kinase, carbohydrate kinase, colanic acid biosynthesis protein, coproporphyrinogen oxidase, cyclin D , DNA polymerase subunit, electron transfer flavoprotein subunit beta, geranylgeranyl pyrophosphate synthase, geranyltranstransferase, gluconate transport system permease 3, glucose dehydrogenase, glucose-6-phosphate 1-dehydrogenase, glutaredoxin, glycerol dehydrogenase, glycerol-3-phosphate dehydrogenase, glycolate utilization protein, harpin-induced family protein, homocitrate synthase, hydrolase, isopropylmalate isomerase large subunit, lipopolysaccharide biosynthesis protein, MADS box protein transcription factor, major facilitator superfamily transporter protein, malate dehydrogenase, monothiol glutaredoxin, monthiol glutaredoxin, multiple antibiotic resistance protein, murein transglycosylase, N-(5′-phosphoribosyl)anthranilate isomerase, NAD-dependent epimerase/dehydratase, NADP-dependent malic enzyme, O-antigen chain length determinant, phosphatidylserine decarboxylase, phospho-2-dehydro-3-deoxyheptonate aldolase, phosphoadenosine phosphosulfate reductase , phosphofructokinase, photosystem II protein, polygalacturonase, polyphosphate kinase, precorrin methylase, protein kinase, protein phosphatase, putative transport system permease protein, pyruvate kinase, RNAbinding protein, Sec-independent protein translocase subunit, serine protease, sll1761-protein, slr1917-protein, small nuclear ribonucleoprotein , ThiF family protein, thioredoxin, thioredoxin family protein, threonine synthase, transcription factor, transcriptional regulator, transport protein, trehalose-phosphatase, TTC1386-protein, ycl049c-protein, ydl235c-protein, ydr183w-protein, yer106w-protein, ylr178c-protein, ypr170c-protein, and zinc finger protein, or of a polypeptide as indicated in the respective line in Table II, application no. 7, columns 5 or 8, or its homologs or fragments, and conferring the production of or an increase in alpha-tocopherol, or gamma-tocopherol, respectively; and/or
(b) stabilizing a mRNA conferring the generation or increased expression of a FCRP, e.g. protein encoded by the nucleic acid molecule of the invention or its homologs or fragments, or of a mRNA encoding the polypeptide of the present invention having the hereinmentioned activity selected from the group consisting of said activities mentioned in (a) and conferring the production of or an increase in alpha-tocopherol, or gamma-tocopherol, respectively; and/or
(c) increasing the specific activity of a protein conferring the increased expression of a FCRP, e.g. a protein encoded by the nucleic acid molecule of the invention or of the polypeptide of the present invention having herein-mentioned alpha-tocopherol, or gamma-tocopherol generating or increasing activity, respectively, e.g. of a polypeptide having the activity of a protein as indicated in the respective line in Table II, application no. 7, columns 5 or 8, or its homologs or fragments, or decreasing the inhibitory regulation of the polypeptide of the invention; and/or
(d) generating or increasing the expression of an endogenous or artificial transcription factor mediating the expression of a protein conferring the generation or increased expression of a FCRP, e.g. a protein encoded by the nucleic acid molecule of the invention or of the polypeptide of the invention having herein-mentioned activity selected from the group consisting of said activities mentioned in (a) and conferring a alpha-tocopherol, or gamma-tocopherol increasing activity, respectively, e.g. of a polypeptide having the activity of a protein as indicated in the respective line in Table II, application no. 7, columns 5 or 8 or its homologs; and/or
(e) stimulating activity of a protein conferring the increased expression of a FCRP, e.g. a protein encoded by the nucleic acid molecule of the present invention or a polypeptide of the present invention having herein-mentioned activity selected from the group consisting of said activities mentioned in (a) and conferring a alpha-tocopherol, or gamma-tocopherol increasing activity, respectively, e.g. of a polypeptide having the activity of a protein as indicated in the respective line in Table II, application no. 7, columns 5 or 8, or its homologs or fragments, by adding one or more exogenous inducing factors to the non-human organism or parts thereof; and/or
(f) expressing a transgenic gene encoding a protein conferring the increased expression of a FCRP, e.g. a polypeptide encoded by the nucleic acid molecule of the present invention or a polypeptide of the present invention, having herein-mentioned activity selected from the group consisting of said activities mentioned in (a) and conferring a alpha-tocopherol, or gamma-tocopherol increasing activity, respectively, e.g. of a polypeptide having the activity of a protein as indicated in the respective line in Table II, application no. 7, columns 5 or 8, or its homologs or fragments, and/or
(g) increasing the copy number of a gene conferring the increased expression of a nucleic acid molecule encoding a FCRP, e.g. a polypeptide encoded by the nucleic acid molecule of the invention or the polypeptide of the invention having herein-mentioned activity selected from the group consisting of said activities mentioned in (a) and conferring a alpha-tocopherol, or gamma-tocopherol increasing activity, respectively, e.g. of a polypeptide having the activity of a protein as indicated in the respective line in Table II, application no. 7, columns 5 or 8, or its homologs or fragments; and/or
(h) increasing the expression of the endogenous gene encoding the FCRP, polypeptide of the invention, e.g. a polypeptide encoded by the nucleic acid molecule of the invention or the polypeptide of the invention having herein-mentioned activity selected from the group consisting of said activities mentioned in (a) and conferring a alpha-tocopherol, or gamma-tocopherol; increasing activity, respectively, e.g. of a polypeptide having the activity of a protein as indicated in the respective line in Table II, application no. 7, columns 5 or 8, or its homologs or fragments, by adding positive expression or removing negative expression elements, e.g. homologous recombination can be used to either introduce positive regulatory elements like for plants the 35S enhancer into the promoter or to remove repressor elements form regulatory regions. Further gene conversion methods can be used to disrupt repressor elements or to enhance to activity of positive elements. Positive elements can be randomly introduced in plants by T-DNA or transposon mutagenesis and lines can be identified in which the positive elements have be integrated near to a gene of the invention, the expression of which is thereby enhanced; and/or
(i) modulating growth conditions of the non-human organism in such a manner, that the expression or activity of the gene encoding the FCRP, e.g. a protein of the invention or the protein itself is enhanced, for example microorganisms or plants can be grown for example under a higher temperature regime leading to an enhanced expression of heat shock proteins, which can lead to an enhanced alpha-tocopherol, or gamma-tocopherol production, respectively; and/or
(j) selecting of non-human organisms with especially high activity of the proteins of the invention from natural or from mutagenized resources and breeding them into the target organisms, e.g. the elite crops; and/or
(k) directing a protein encoded by the nucleic acid molecule of the invention or of the polypeptide of the present invention having herein-mentioned activity selected from the group consisting of said activities mentioned in (a) and conferring a alpha-tocopherol, or gamma-tocopherol increasing activity, respectively, e.g. of a polypeptide having the activity of a protein as indicated in the respective line in Table II, application no. 7, columns 5 or 8, or its homologs or fragments, if for the corresponding nucleic acid molecule in the respective line in column 6 of Table I the term “plastidic” is indicated, to the plastids by the addition of a plastidial targeting sequence or if for the corresponding nucleic acid molecule in the respective line in column 6 of Table I the term “mitochondric” is indicated, to the mitochondria by the addition of a mitochondrial targeting sequence; and/or
(l) generating the expression of a protein encoded by the nucleic acid molecule of the invention or of the polypeptide of the present invention having herein-mentioned activity selected from the group consisting of said activities mentioned in (a) and conferring a alpha-tocopherol, or gamma-tocopherol increasing activity, respectively, e.g. of a polypeptide having the activity of a protein as indicated in the respective line in Table II, application no. 7, columns 5 or 8, or its homologs or fragments, if for the corresponding nucleic acid molecule in the respective line in column 6 of Table I the term “plastidic” or “mitochondric” is indicated, in these organelles by the stable or transient transformation, advantageously stable transformation, of organelles, preferably plastids or mitochondria, with an inventive nucleic acid sequence preferably in form of an expression cassette containing said sequence leading to the expression of the nucleic acids or polypeptides of the invention in the respective organelle; and/or
(m) generating the expression of a protein encoded by the nucleic acid molecule of the invention or of the polypeptide of the present invention having herein-mentioned activity selected from the group consisting of said activities mentioned in (a) and conferring a alpha-tocopherol, and/or gamma-tocopherol increasing activity, respectively, e.g. of a polypeptide having the activity of a protein as indicated in the respective line in Table II, application no. 7, columns 5 or 8, or its homologs or fragments, if for the corresponding nucleic acid molecule in the respective line in column 6 of Table I the term “plastidic” or “mitochondric” is indicated, in these organelles by integration of a nucleic acid of the invention into the genome of the respective organelle under control of preferable a promoter selective for the respective organelle.

Preferably, said mRNA is the coding region of the nucleic acid molecule of the present invention and/or the protein conferring the increased expression of a protein encoded by the coding region of the nucleic acid molecule of the present invention alone or linked to a transit nucleic acid sequence or transit peptide encoding nucleic acid sequence or the polypeptide having the herein mentioned activity, e.g. conferring the generation of or increase of alpha-tocopherol, or gamma-tocopherol, respectively, after increasing the expression or activity of the encoded polypeptide, non-targeted or in organelles such as plastids and/or mitochondria, preferably plastids, or having the activity of a polypeptide having an activity as the protein as shown in the respective line in Table II, application no. 7, column 3, or its homologs. Preferably the increase of alpha-tocopherol, or gamma-tocopherol, respectively, takes place non-targeted or in plastids and/or mitochondria, preferably non-targeted or in plastids.

In general, the amount of mRNA or polypeptide in a cell or a compartment of a non-human organism correlates with the amount of encoded protein and thus with the overall activity of the encoded protein in said volume. Said correlation is not always linear, the activity in the volume is dependent on the stability of the molecules, the degradation of the molecules or the presence of activating or inhibiting co-factors. Further, product and educt inhibitions of enzymes are well known and described in Textbooks, e.g. Stryer, Biochemistry or Zinser et al. “Enzyminhibitoren”/Enzyme inhibitors”.

The activity of the above-mentioned proteins and/or polypeptide encoded by the nucleic acid molecule of the present invention can be increased in various ways. For example, the activity in an organism or in a part thereof, like a cell or in an organelle of the cell, is increased for example via targeting of the nucleic acid sequence or the encoded gene product to an organelle such as plastids or mitochondria, preferentially to plastids, and/or increasing the gene product number, e.g. by increasing the expression rate, like introducing a stronger promoter, or by increasing the stability of the mRNA expressed, thus increasing the translation rate, and/or increasing the stability of the gene product, thus reducing the proteins decayed. Further, the activity or turnover of enzymes can be influenced in such a way that a reduction or increase of the reaction rate or a modification (reduction or increase) of the affinity to the substrate results, is reached. A mutation in the catalytic centre of an polypeptide of the invention, e.g. as enzyme, can modulate the turn over rate of the enzyme, e.g. an exchange of an amino acid in the catalytic center can lead to an increased activity of the enzyme, or the deletion of regulator binding sites can reduce a negative regulation like a feedback inhibition (or a substrate inhibition, if the substrate level is also increased). The specific activity of an enzyme of the present invention can be increased such that the turn over rate is increased or the binding of a co-factor is improved. Improving the stability of the encoding mRNA or the protein can also increase the activity of a gene product. The stimulation of the activity is also under the scope of the term “increased activity”.

Moreover, the regulation of the above-mentioned nucleic acid sequences may be modified so that gene expression is increased. This can be achieved advantageously by means of heterologous regulatory sequences or by modifying, for example mutating, the natural regulatory sequences which are present. The advantageous methods may also be combined with each other.

In general, an activity of a gene product in a non-human organism or part thereof, in particular in a plant cell or organelle of a plant cell, a plant, or a plant or a part thereof or in a microorganism can be increased by increasing the amount of the specific encoding mRNA or the corresponding protein in said organism or part thereof. “Amount of protein or mRNA” is understood as meaning the molecule number of polypeptides or mRNA molecules in an organism, a tissue, a cell or a cell compartment. “Increase” in the amount of a protein means the quantitative increase of the molecule number of said protein in a non-human organism, a cell or a cell compartment, such as an organelle like a plastid or mitochondrion, or part thereof for example by one of the methods described herein below - in comparison to a wild type, controt or reference.

The increase in molecule number amounts preferably to at least 1%, 5%, 10%, 20%, 30%, 50%, 70%, 100%, 150%, 200% or more. However, a de novo expression is also regarded as subject of the present invention.

A modification, i.e. an increase, can be caused by endogenous or exogenous factors. For example, an increase in activity in a non-human organism or a part thereof can be caused by adding a gene product or a precursor or an activator or an agonist to the media or nutrition or can be caused by introducing said subjects into an organism, transient or stable. Such an increase can be reached by the introduction of the inventive nucleic acid sequence or the encoded protein into an organism, transient or stable. Furthermore such an increase can be reached by the introduction of the inventive nucleic acid sequence, preferably the coding region thereof, or the encoded protein in the correct cell compartment, for example into organelles, such as plastids or mitochondria, either by transformation and/or targeting.

In one embodiment the increase in the amount of the fine chemical in the non-human organism or a part thereof, e.g. in a cell, an organ, an organelle etc., is achieved by increasing the endogenous level of the polypeptide of the invention. Accordingly, in an embodiment of the present invention, the present invention relates to a process wherein the gene copy number of a gene encoding the polynucleotide or nucleic acid molecule of the invention is increased. Further, the endogenous level of the polypeptide of the invention can for example be increased by modifying the transcriptional or translational regulation of the polypeptide.

In one embodiment the amount of the fine chemical in the non-human organism or part thereof can be increase by targeted or random mutagenesis of the endogenous genes of the invention. For example homologous recombination can be used to either introduce positive regulatory elements like for plants the 35S enhancer into the promoter or to remove repressor elements form regulatory regions. In addition gene conversion like methods described by Kochevenko and Willmitzer (Plant Physiol. 132 (1), 174 (2003)) and citations therein can be used to disrupt repressor elements or to enhance to activity of positive regulatory elements.

Furthermore positive elements can be randomly introduced in (plant) genomes by T-DNA or transposon mutagenesis and lines can be screened for, in which the positive elements has be integrated near to a gene of the invention, the expression of which is thereby enhanced. The activation of plant genes by random integrations of enhancer elements has been described by Hayashi et al. (Science 258,1350 (1992)) or Weigel et al. (Plant Physiol. 122, 1003 (2000)) and others citated therein.

Reverse genetic strategies to identify insertions (which eventually carrying the activation elements) near in genes of interest have been described for various cases e.g. Krysan et al. (Plant Cell 11, 2283 (1999)); Sessions et al. (Plant Cell 14, 2985 (2002)); Young et al. (Plant Physiol. 125, 513 (2001)); Koprek et al. (Plant J. 24, 253 (2000)); Jeon et al.(Plant J. 22, 561 (2000)); Tissier et al., (Plant Cell 11, 1841 (1999)); Speulmann et al. (Plant Cell 11, 1853 (1999)). Briefly, material from all plants of a large T-DNA or transposon mutagenized plant population is harvested and genomic DNA prepared. Then the genomic DNA is pooled following specific architectures as described for example in Krysan et al. (Plant Cell 11, 2283 (1999)). Pools of genomics DNAs are then screened by specific multiplex PCR reactions detecting the combination of the insertional mutagen (e.g. T-DNA or Transposon) and the gene of interest. Therefore PCR reactions are run on the DNA pools with specific combinations of T-DNA or transposon border primers and gene specific primers. General rules for primer design can again be taken from Krysan et al. (Plant Cell 11, 2283 (1999)). Rescreening of lower levels DNA pools lead to the identification of individual plants in which the gene of interest is disrupted by the insertional mutagen.

The enhancement of positive regulatory elements or the disruption or weaking of negative regulatory elements can also be achieved through common mutagenesis techniques: The production of chemically or radiation mutated populations is a common technique and known to the skilled worker. Methods for plants are described by Koorneef et al. 1982 and the citations therein and by Lightner and Caspar in “Methods in Molecular Biology” Vol. 82. These techniques usually induce point mutations that can be identified in any known gene using methods such as TILLING (Colbert et al., Plant Physiol.126 (2), 480 (2001)). One can also envisage to introduce nucleic acids sequences, encoding organelle-, such as plastidal- or mitochondrial-targeting signals, like for example present in Table a or Table b, respectively, by homologous recombination or other methods of site specific integration, into the genome in that way, that an endogenous gene is functionally fused to the targeting sequence and the protein is redirected to the plastids or mitochondria, respectively. Eventually the integration can also occur randomly and the desired fusion event is selected for.

Accordingly, the expression level can be increased if the endogenous genes encoding a polypeptide conferring an increased expression of the polypeptide of the present invention, in particular genes comprising the nucleic acid molecule of the present invention, are modified via homologous recombination, Tilling approaches or gene conversion. It is also possible to add as mentioned herein targeting sequences to the inventive nucleic acid sequences.

Regulatory sequences, preferably in addition to a targeting sequence or part thereof (if present), can be operatively linked to the coding region of an endogenous nucleic acid and control its transcription and translation or the stability or decay of the encoding mRNA or the expressed protein. In order to modify and control the expression, promoter, UTRs, splicing sites, processing signals, polyadenylation sites, terminators, enhancers, repressors, post transcriptional or posttranslational modification sites can be changed, added or amended. For example, the activation of plant genes by random integrations of enhancer elements has been described by Hayashi et al. (Science 258, 1350 (1992)) or Weigel et al. (Plant Physiol. 122, 1003 (2000)) and others citated therein. For example, the expression level of the endogenous protein can be modulated by replacing the endogenous promoter with a stronger transgenic promoter or by replacing the endogenous 3′UTR with a 3′UTR, which provides more stability without amending the coding region. Further, the transcriptional regulation can be modulated by introduction of an artificial transcription factor as described in the examples. Alternative promoters, terminators and UTR are described below.

The activation of an endogenous polypeptide having above-mentioned activity, e.g. having the activity of a protein as shown in the respective line in Table II, application no. 7, column 3 or of the polypeptide of the invention, e.g. conferring the increase of the fine chemical alpha-tocopherol, or gamma-tocopherol, respectively, by increase of expression or activity in the cytoplasm, and/or in the cytosol, and/or in an organelle, such as plastids or mitochondria, can also be increased by introducing a synthetic transcription factor, which binds close to the coding region of the gene encoding the protein as shown in the respective line in Table II, application no. 7, column 5 or 8, or homologs or fragments thereof, and activates its transcription. A chimeric zinc finger protein can be constructed, which comprises a specific DNA-binding domain and an activation domain as e.g. the VP16 domain of Herpes Simplex virus. The specific binding domain can bind to the regulatory region of the gene encoding the protein as shown in the respective line in Table II, application no. 7, column 5 or 8, or homologs or fragments thereof. The expression of the chimeric transcription factor in a non-human organism, in particular in a plant, leads to a specific expression of the protein as shown in the respective line in Table II, application no. 7, column 5 or 8, or homologs or fragments thereof, see e.g. in WO01/52620, Oriz, Proc. Natl. Acad. Sci. USA 99, 13290 (2002), or Guan, Proc. Natl. Acad. Sci. USA 99, 13296 (2002).

In one further embodiment of the process according to the invention, organisms are used in which one of the above-mentioned genes, or one of the above-mentioned nucleic acids, is mutated in a way that the activity of the encoded gene products is less influenced by cellular factors, or not at all, in comparison with the unmutated proteins. For example, well known regulation mechanisms of enzymic activity are substrate inhibition or feed back regulation mechanisms. Ways and techniques for the introduction of substitution, deletions and additions of one or more bases, nucleotides or amino acids of a corresponding sequence are described herein below in the corresponding paragraphs and the references listed there, e.g. in Sambrook et al., Molecular Cloning, Cold Spring Habour, N.Y., 1989. The person skilled in the art will be able to identify regulation domains and binding sites of regulators by comparing the sequence of the nucleic acid molecule of the present invention or the expression product thereof with the state of the art by computer software means which comprise algorithms for the identifying of binding sites and regulation domains or by introducing into a nucleic acid molecule or in a protein systematically mutations and assaying for those mutations which will lead to an increased specify activity or an increased activity per volume, in particular per cell.

It can therefore be advantageous to express in a non-human organism a nucleic acid molecule of the invention or a polypeptide of the invention derived from a evolutionary distantly related organism, as e.g. using a prokaryotic gene in a eukaryotic host, as in these cases the regulation mechanism of the host cell may not weaken the activity (cellular or specific) of the gene or its expression product.

In another embodiment it can be advantageous to express in a non-human organism a nucleic acid molecule of the invention or a polypeptide of the invention in a compartment by the use of a respective targeting sequence, in which it is naturally not expressed, as in these cases the regulation mechanism of the host cell may not weaken the activity (cellular or specific) of the gene or its expression product.

The mutation is introduced in such a way that the production of the fine chemical is not adversely affected by e.g. undesired mutations.

Less influence on the regulation of a gene or its gene product is understood as meaning a reduced regulation of the enzymatic activity leading to an increased specific or cellular activity of the gene or its product. An increase of the enzymatic activity is understood as meaning an enzymatic activity, which is increased by at least 10%, 20%, 30%, 40%, 50%, 60% or 70% in comparison with the starting organism. This leads to an increased productivity of the desired fine chemical(s).

Owing to the introduction of a gene or a plurality of genes conferring the expression of the nucleic acid molecule of the invention or the polypeptide of the invention, for example the nucleic acid construct mentioned below, or encoding the protein as shown in the respective line in Table II, application no. 7, column 5 or 8, or homologs or fragments thereof, into a non-human organism alone or in combination with other genes, it is possible not only to increase the biosynthetic flux towards the end product, but also to increase, modify or create de novo an advantageous, preferably novel metabolites composition in the non-human organism, e.g. an advantageous composition comprising a higher content of (from a viewpoint of nutritional physiology limited) Vitamin E, alpha-tocopherol, or gamma-tocopherol and if desired other vitamins, and/or other metabolites, in free or bound form.

In another embodiment the composition comprises further higher amounts of metabolites positively affecting or lower amounts of metabolites negatively affecting the nutrition or health of animals or humans provided with said compositions or non-human organisms of the invention or parts thereof. Likewise, the number or activity of further genes which are required for the import or export of nutrients or metabolites, including amino acids, fatty acids, vitamins etc. or its precursors, required for the cell's biosynthesis of the fine chemical may be increased so that the concentration of necessary or relevant precursors, cofactors or intermediates within the cell(s) or within the corresponding storage compartments is increased. Owing to the increased or novel generated activity of the polypeptide of the invention or owing to the increased number of nucleic acid sequences of the invention and/or to the modulation of further genes which are involved in the biosynthesis of the fine chemical, e.g. by increasing the activity of enzymes synthesizing precursors or by destroying the activity of one or more genes which are involved in the breakdown of the fine chemical, it is possible to increase the yield, production and/or production efficiency of the fine chemical in the host organism, such as plants or microorganisms.

Accordingly, in one embodiment, the process according to the invention relates to a process, which comprises:
(a) providing a non-human organism, preferably a microorganism, a plant cell, a plant tissue, a plant or a part thereof, more preferably a microorganism, a plant tissue, a plant or a part thereof;
(b) increasing the activity of a protein as shown in the respective line in Table II, application no. 7, column 3 or of a polypeptide being encoded by the respective nucleic acid molecule of the present invention and described below, e.g. conferring an increase of the fine chemical Vitamin E, alpha-tocopherol, or gamma-tocopherol, respectively, in the non-human organism, preferably in the microorganism, the plant cell, the plant tissue, the plant or a part thereof, more preferably a microorganism, a plant tissue, a plant or a part thereof, especially cytoplasmic or in an organelle, like plastids or mitochondria,
(c) growing the non-human organism, preferably the microorganism, the plant cell, the plant tissue, the plant or a part thereof, under conditions which permit the production of said chemical in the non-human organism, preferably the microorganism, the plant cell, the plant tissue, the plant or a part thereof; and
(d) if desired, recovering, optionally isolating, said fine chemical, in free and/or bound form, and, optionally further free and/or bound vitamins, and/or other metabolites synthesized by the non-human organism, the microorganism, the plant cell, the plant tissue, the plant or a part thereof.

After the above-described increasing (which as defined above also encompasses the generating of an activity in a non-human organism, i.e. a de novo activity), for example after the introduction and the expression of the nucleic acid molecules of the invention or described in the methods or processes according to the invention, the organism according to the invention, advantageously, a microorganism, a non-human animal, a plant, plant or animal tissue or plant or animal cell, is grown and subsequently harvested.

Suitable non-human organisms or host organisms (transgenic organism) for the nucleic acid molecule used according to the invention and for the inventive process, the nucleic acid construct or the vector (both as described below) are, in principle, all organisms which are capable of synthesizing the fine chemical, and which are suitable for the activation, introduction or stimulation of recombinant genes. Examples which may be mentioned are transgenic plants, transgenic microorganisms such as fungi, bacteria, yeasts, alga or diatom preferably alga. Preferred organisms are those which are naturally capable of synthesizing the fine chemical in substantial amounts, like fungi, yeasts, bacteria or plants preferably alga and plants.

In the event that the transgenic non-human organism is a microorganism, such as a eukaryotic organism, for example a fungus, an alga, diatom or a yeast, in particular a fungus, alga, diatom or yeast selected from the families Tuberculariaceae, Adelotheciaceae, Dinophyceae, Ditrichaceae or Prasinophyceae. Preferred non-human organisms are microorganisms such as green algae or plants. After the growing phase, the organisms can be harvested.

The non-human organism, in particular the microorganism, plant or part thereof, is advantageously grown in such a way that it is not only possible to recover, if desired to isolate the free or bound fine chemical or the free and bound fine chemical but as option it is also possible to produce, recover and, if desired to isolate, other free or/and bound vitamins and/or other metabolites.

The organisms are harvested in a manner known per se and appropriate for the particular organism. Microorganisms such as bacteria, mosses, yeasts and fungi or plant cells which are cultured in liquid media by fermentation may be removed, for example, by centrifugation, decanting or filtration. Plants are grown on solid media in a manner known per se and harvested accordingly.

Vitamin E or its precursor 2,3-dimethyl-5-phytylquinol are isolated from the harvested biomass in a manner known per se, for example by extraction and, where appropriate, further chemical or physical purification processes such as, for example, precipitation methods, crystallography, thermal separation methods such as rectification methods or physical separation methods such as, for example, chromatography.

Vitamin E is isolated from oil-containing plants, for example, preferably by chemical conversion and distillation from vegetable oils or from the steam distillates obtained in the deodorization of vegetable oils (deodorizer condensates).

Further methods of isolating vitamin E from deodorizer condensates are described, for example, in DE 31 26 110 A1, EP 171 009 A2, GB 2 145 079, EP 333 472 A2 and WO 94/05650.

Preferred microorganisms are selected from the group consisting of Charophyceae such as the genera Chara, Nitella e.g. the species Chara globularis, Chara vulgaris, Nitella flexilis, Chlorophyceae such as the genera Acrosiphonia, Spongomorpha, Urospora, Bryopsis, Pseudobryopsis, Trichosolen, Dichotomosiphon, Caulerpa, Rhipilia, Blastophysa, Avrainvillea, Chlorodesmis, Codium, Espera, Halicystis, Halimeda, Penicillus, Pseudocodium, Rhipiliopsis, Rhipocephalus, Tydemania, Udotea, Derbesia, Acrochaete, Aphanochaete, Bolbocoleon, Chaetobolus, Chaetonema, Chaetophora, Chlorotylium, Desmococcus, Draparnaldia, Draparnaldiopsis, Ectochaete, Endophyton, Entocladia, Epicladia, Internoretia, Microthamnion, Ochlochaete, Phaeophila, Pilinella, Pringsheimiella, Protoderma, Pseudendoclonium, Pseudodictyon, Pseudopringsheimia, Pseudulvella, Schizomeris, Stigeoclonium, Thamniochaete, Ulvella, Pilinia, Tellamia, Helicodictyon, Actidesmium, Ankyra, Characium, Codiolum, Sykidion, Keratococcus, Prototheca, Bracteacoccus Chlorococcum, Excentrosphaera, Hormidium, Oophila, Schroederia, Tetraedron, Trebouxia Chlorosarcinopsis, Gomphonitzschia, Coccomyxa, Dactylothece, Diógenes, Disporá, Gloeocystis, Mycanthococcus, Ourococcus, Coelastrum, Dicranochaete, Botryococcus, Dictyosphaerium, Dimorphococcus, Chlorochytrium, Kentrosphaera, Phyllobium, Gomontia, Hormotila, Euastropsis, Hydrodictyon, Pectodictyon, Pediastrum, Sorastrum, Tetrapedia, Acanthosphaera, Echinosphaerella, Echinosphaeridium, Errerella, Gloeoactinium, Golenkeniopsis Golenkinia, Micractinium, Ankistrodesmus, Chlorella, Chodatella, Closteriopsis, Cryocystis, Dactylococcus, Dematractum, Eremosphaera, Eutetramorus, Franceia, Glaucocystis, Gloeotaenium, Kirchneriella, Lagerheimiella, Monoraphidium, Nannochloris, Nephrochlamys, Nephrocytium, Oocystis, Oonephris, Pachycladon, Palmellococcus, Planktosphaeria, Polyedriopsis, Pseudoraciborskia, Quadrigula, Radiococcus, Rochiscia, Scotiella, Selanastrum, Thorakochloris, Treubaria, Trochiscia, Westella, Zoochlorella, Ostreobium, Phyllosiphon, Protosiphon, Rhodochytrium, Actinastrum, Coronastrum, Crucigenia, Dictymocystis, Enallax, Scenedesmus, Selenastrum, Tetradesmus, Tetrallantos, Tetrastrum, Chlorosarcina, Anadyomene, Valoniopsis, Ventricaria, Basicladia, Chaetomorpha, Cladophora, Lola Pithophora Rhizoclonium Chaetosphaeridium, Conochaete, Coleochaete, Oligochaetophora, Polychaetophora, Cylindrocapsa, Gongrosira, Protococcus, Acetabularia, Batophora, Bornetella, Dasycladus, Halicoryne, Neomeris, Elakatothrix, Raphidonema, Microspora, Bulbochaete, Oedocladium, Oedogonium, Prasiola, Rosenvingiella, Schizogonium, Apjohnia, Chamaedoris, Cladophoropsis, Siphonocladus, Spongocladia, Boergesenia, Boodlea, Cystodictyon, Dictyosphaeria, Ernodesmis, Microdictyon, Struvea, Valonia, Sphaeroplea, Malleochloris, Stylosphaeridium, Gloeococcus, Palmella, Palmodictyon, Palmophyllum, Pseudospherocystis, Sphaerocystis, Urococcus, Apiocystis, Chaetopeltis, Gemellicystis, Paulschulzia, Phacomyxa, Pseudotetraspora, Schizochlamys, Tetraspora, Cephaleuros, Ctenocladus, Epibolium, Leptosira, Trentepohlia, Diplochaete, Monostroma, Binuclearia, Geminella, Klebsormidium, Planetonema, Radiofilum, Stichococcus, Ulothrix, Uronema, Blidingia, Capsosiphon, Chloropelta, Enteromorpha, Percursaria, Ulva, Ulvaria, Brachiomonas, Carteria, Chlainomonas, Chlamydomonas, Chlamydonephris, Chlorangium, Chlorogonium, Cyanidium, Fortiella, Glenomonas, Gloeomonas, Hyalogonium, Lobomonas Polytoma, Pyramichlamys, Scourfieldia, Smithsonimonas, Sphaerellopsis, Sphenochloris, Spirogonium, Collodictyon, Dunaliella, Haematococcus, Stephanosphaera, Coccomonas, Dysmorphococcus, Phacotus, Pteromonas, Thoracomonas, Wislouchiella, Mascherina, Pyrobotrys, Spondylomorum, Eudorina, Gonium, Oltmannsiella, Pandorina, Platydorina, Pleodorina, Stephanoon, Volvox, Volvulina, Actinotaenium, Arthrodesmus, Bambusina Closterium, Cosmarium, Desmidium, Euastrum, Groenbladia, Hyalotheca, Micrasterias, Penium, Phymatodocis, Pleurotaenium, Sphaerozosma, Spinoclosterium, Spinocosmarium, Spondylosium, Staurastrum, Tetmemorus, Triploceras, Xanthidium, Cylindrocystis, Genicularia, Gonatozygon, Mesotaenium, Netrium, Roya, Spirotaenia, Cosmocladium, Debarya, Docidium, Euastridium, Hallasia, Mougeotia, Mougeotiopsis, Sirogonium, Spirogyra, Staurodesmus, Teilingia, Zygnema, Zygogonium, e.g. the species Caulerpa taxifolia, Prototheca wickerhamii, Ankistrodesmus falcatus, Chlorella ellipsoidea, Chlorella pyrenoidosa, Clorella sorokiniana, Chlorella vulgaris, Scenedesmus obliquus, Scenedesmus quadricauda, Selenastrum capricornutum, Selenastrum undecimnotata, Cladophora glomerata, Chlamydomonas eugametos, Chlamydomonas reinhardtii, Cyanidium caldarium, Dunaliella salina, Dunaliella tertiolecta, Euglena gracilis, Haematococcus pluvialis, Coniugatophyceae, Prasinophyceae Trebouxiophyceae, Ulvophyceae, Chlorodendraceae, Pedinomonadales, Halosphaeraceae, Pterospermataceae, Monomastigaceae, Pyramimonadaceae, Chlorodendraceae such as the genera Prasinocladus e.g. the species Prasinocladus ascus, Halosphaeraceae, Pedinomonadales, Pedinomonadaceae such as the genera Pedinomonas, Pterospermataceae such as the genera Pachysphaera, Pterosperma, Halosphaera, Pyramimonas, Bacillariophyceae, Chrysophyceae, Craspedophyceae, Euglenophyceae, Prymnesiophyceae, Phaeophyceae, Dinophyceae, Rhodophyceae, Xanthophyceae, Prasinophyceae such as the genera Nephroselmis, Prasinococcus, Scherffelia, Tetraselmis, Mantoniella, Ostreococcus e.g. the species Nephroselmis olivacea, Prasinococcus capsulatus, Scherffelia dubia, Tetraselmis chui, Tetraselmis suecica, Mantoniella squamata or Ostreococcus tauri.

Preferred plants are monocotyledonous or dicotyledonous plants, preferably selected from the group consisting of Anacardiaceae such as the genera Pistacia, Mangifera, Anacardium e.g. the species Pistacia vera [pistachios, Pistazie], Mangifer indica [Mango] or Anacardium occidentale [Cashew]; Asteraceae such as the genera Calendula, Carthamus, Centaurea, Cichorium, Cynara, Helianthus, Lactuca, Locusta, Tagetes, Valeriana e.g. the species Calendula officinalis [Marigold], Carthamus tinctorius [safflower], Centaurea cyanus [cornflower], Cichorium intybus [blue daisy], Cynara scolymus [Artichoke], Helianthus annus [sunflower], Lactuca sativa, Lactuca crispa, Lactuca esculenta, Lactuca scariola L. ssp. sativa, Lactuca scariola L. var. integrate, Lactuca scariola L. var. integrifolia, Lactuca sativa subsp. romana, Locusta communis, Valeriana locusta [lettuce], Tagetes lucida, Tagetes erecta or Tagetes tenuifolia [Marigold]; Apiaceae such as the genera Daucus e.g. the species Daucus carota [carrot]; Betulaceae such as the genera Corylus e.g. the species Corylus avellana or Corylus colurna [hazelnut]; Boraginaceae such as the genera Borago e.g. the species Borago officinalis [borage]; Brassicaceae such as the genera Brassica, Melanosinapis, Sinapis, Arabadopsis e.g. the species Brassica napus, Brassica rapa ssp. [canola, oilseed rape, turnip rape], Sinapis arvensis Brassica juncea, Brassica juncea var. juncea, Brassica juncea var. crispifolia, Brassica juncea var. foliosa, Brassica nigra, Brassica sinapioides, Melanosinapis communis [mustard], Brassica oleracea [fodder beet] or Arabidopsis thaliana; Bromeliaceae such as the genera Anana, Bromelia e.g. the species Anana comosus, Ananas ananas or Bromelia comosa [pineapple]; Caricaceae such as the genera Carica e.g. the species Carica papaya [papaya]; Cannabaceae such as the genera Cannabis e.g. the species Cannabis sative [hemp], Convolvulaceae such as the genera Ipomea, Convolvulus e.g. the species Ipomoea batatus, Ipomoea pandurata, Convolvulus batatas, Convolvulus tiliaceus, Ipomoea fastigiata, Ipomoea tiliacea, Ipomoea triloba or Convolvulus panduratus [sweet potato, Man of the Earth, wild potato], Chenopodiaceae such as the genera Beta, i.e. the species Beta vulgaris, Beta vulgaris var. altissima, Beta vulgaris var. Vulgaris, Beta maritima, Beta vulgaris var. perennis, Beta vulgaris var. conditiva or Beta vulgaris var. esculenta [sugar beet]; Cucurbitaceae such as the genera Cucubita e.g. the species Cucurbita maxima, Cucurbita mixta, Cucurbita pepo or Cucurbita moschata [pumpkin, squash]; Elaeagnaceae such as the genera Elaeagnus e.g. the species Olea europaea [olive]; Ericaceae such as the genera Kalmia e.g. the species Kalmia latifolia, Kalmia angustifolia, Kalmia microphylla, Kalmia polifolia, Kalmia occidentalis, Cistus chamaerhodendros or Kalmia lucida [American laurel, broad-leafed laurel, calico bush, spoon wood, sheep laurel, alpine laurel, bog laurel, western bog-laurel, swamp-laurel]; Euphorbiaceae such as the genera Manihot, Janipha, Jatropha, Ricinus e.g. the species Manihot utilissima, Janipha manihot, Jatropha manihot, Manihot aipil, Manihot dulcis, Manihot manihot, Manihot melanobasis, Manihot esculenta [manihot, arrowroot, tapioca, cassava] or Ricinus communis [castor bean, Castor Oil Bush, Castor Oil Plant, Palma Christi, Wonder Tree]; Fabaceae such as the genera Pisum, Albizia, Cathormion, Feuillea, Inga, Pithecolobium, Acacia, Mimosa, Medicajo, Glycine, Dolichos, Phaseolus, Soja e.g. the species Pisum sativum, Pisum arvense, Pisum humile [pea], Albizia berteriana, Albizia julibrissin, Albizia lebbeck, Acacia berteriana, Acacia littoralis, Albizia berteriana, Albizzia berteriana, Cathormion berteriana, Feuillea berteriana, Inga fragrans, Pithecellobium berterianum, Pithecellobium fragrans, Pithecolobium berterianum, Pseudalbizzia berteriana, Acacia julibrissin, Acacia nemu, Albizia nemu, Feuilleea julibrissin, Mimosa julibrissin, Mimosa speciosa, Sericanrda julibrissin, Acacia lebbeck, Acacia macrophylla, Albizia lebbek, Feuilleea lebbeck, Mimosa lebbeck, Mimosa speciosa [bastard logwood, silk tree, East Indian Walnut], Medicago sativa, Medicago falcata, Medicago varia [alfalfa], Glycine max [soybean], Dolichos soja, Glycine gracilis, Glycine hispida, Phaseolus max or Soja hispida; Geraniaceae such as the genera Pelargonium, Cocos, Oleum e.g. the species Cocos nucifera, Pelargonium grossularioides or Oleum cocois [coconut]; Gramineae such as the genera Saccharum e.g. the species Saccharum officinarum; Juglandaceae such as the genera Juglans, Wallia e.g. the species Juglans regia, Juglans ailanthifolia, Juglans sieboldiana, Juglans cinerea, Wallia cinerea, Juglans bixbyi, Juglans californica, Juglans hindsii, Juglans intermedia, Juglans jamaicensis, Juglans major, Juglans microcarpa, Juglans nigra or Wallia nigra [walnut, black walnut, common walnut, persian walnut, white walnut, butternut, black walnut]; Lauraceae such as the genera Persea, Laurus e.g. the species laurel Laurus nobilis [bay, laurel, bay laurel, sweet bay], Persea americana Persea americana, Persea gratissima or Persea persea [avocado]; Leguminosae such as the genera Arachis e.g. the species Arachis hypogaea [peanut]; Linaceae such as the genera Linum, Adenolinum e.g. the species Linum usitatissimum, Linum humile, Linum austriacum, Linum bienne, Linum angustifolium, Linum catharticum, Linum flavum, Linum grandiflorum, Adenolinum grandiflorum, Linum lewisii, Linum narbonense, Linum perenne, Linum perenne var. lewisii, Linum pratense or Linum trigynum [flax, linseed]; Lythrarieae such as the genera Punica e.g. the species Punica granatum [pomegranate]; Malvaceae such as the genera Gossypium e.g. the species Gossypium hirsutum, Gossypium arboreum, Gossypium barbadense, Gossypium herbaceum or Gossypium thurberi [cotton]; Musaceae such as the genera Musa e.g. the species Musa nana, Musa acuminata, Musa paradisiaca, Musa spp. [banana]; Onagraceae such as the genera Camissonia, Oenothera e.g. the species Oenothera biennis or Camissonia brevipes [primrose, evening primrose]; Palmae such as the genera Elacis e.g. the species Elaeis guineensis [oil plam]; Papaveraceae such as the genera Papaver e.g. the species Papaver orientale, Papaver rhoeas, Papaver dubium [poppy, oriental poppy, corn poppy, field poppy, shirley poppies, field poppy, long-headed poppy, long-pod poppy]; Pedaliaceae such as the genera Sesamum e.g. the species Sesamum indicum [sesame];

Piperaceae such as the genera Piper, Artanthe, Peperomia, Steffensia e.g. the species Piper aduncum, Piper amalago, Piper angustifolium, Piper auritum, Piper betel, Piper cubeba, Piper longum, Piper nigrum, Piper retrofractum, Artanthe adunca, Artanthe elongata, Peperomia elongata, Piper elongatum, Steffensia elongata. [Cayenne pepper, wild pepper]; Poaceae such as the genera Hordeum, Secale, Avena, Sorghum, Andropogon, Holcus, Panicum, Oryza, Zea, Triticum e.g. the species Hordeum vulgare, Hordeum jubatum, Hordeum murinum, Hordeum secalinum, Hordeum distichon Hordeum aegiceras, Hordeum hexastichon, Hordeum hexastichum, Hordeum irregulare, Hordeum sativum, Hordeum secalinum [barley, pearl barley, foxtail barley, wall barley, meadow barley], Secale cereale [rye], Avena sativa, Avena fatua, Avena byzantina, Avena fatua var. sativa, Avena hybrida [oat], Sorghum bicolor, Sorghum halepense, Sorghum saccharatum, Sorghum vulgare, Andropogon drummondii, Holcus bicolor, Holcus sorghum, Sorghum aethiopicum, Sorghum arundinaceum, Sorghum caffrorum, Sorghum cernuum, Sorghum dochna, Sorghum drummondii, Sorghum durra, Sorghum guineense, Sorghum lanceolatum, Sorghum nervosum, Sorghum saccharatum, Sorghum subglabrescens, Sorghum verticilliflorum, Sorghum vulgare, Holcus halepensis, Sorghum miliaceum millet, Panicum militaceum [Sorghum, millet], Oryza sativa, Oryza latifolia [rice], Zea mays [corn, maize] Triticum aestivum, Triticum durum, Triticum turgidum, Triticum hybernum, Triticum macha, Triticum sativum or Triticum vulgare [wheat, bread wheat, common wheat], Proteaceae such as the genera Macadamia e.g. the species Macadamia intergrifolia [macadamia]; Rubiaceae such as the genera Coffea e.g. the species Cofea spp., Coffea arabica, Coffea canephora or Coffea liberica [coffee]; Scrophulariaceae such as the genera Verbascum e.g. the species Verbascum blattaria, Verbascum chaixii, Verbascum densiflorum, Verbascum lagurus, Verbascum longifolium, Verbascum lychnitis, Verbascum nigrum, Verbascum olympicum, Verbascum phlomoides, Verbascum phoenicum, Verbascum pulverulentum or Verbascum thapsus [mullein, white moth mullein, nettle-leaved mullein, dense-flowered mullein, silver mullein, long-leaved mullein, white mullein, dark mullein, greek mullein, orange mullein, purple mullein, hoary mullein, great mullein]; Solanaceae such as the genera Capsicum, Nicotiana, Solanum, Lycopersicon e.g. the species Capsicum annuum, Capsicum annuum var. glabriusculum, Capsicum frutescens [pepper], Capsicum annuum [paprika], Nicotiana tabacum, Nicotiana alata, Nicotiana attenuata, Nicotiana glauca, Nicotiana langsdorffii, Nicotiana obtusifolia, Nicotiana quadrivalvis, Nicotiana repanda, Nicotiana rustica, Nicotiana sylvestris [tobacco], Solanum tuberosum [potato], Solanum melongena [egg-plant] (Lycopersicon esculentum, Lycopersicon lycopersicum, Lycopersicon pyriforme, Solanum integrifolium or Solanum lycopersicum [tomato]; Sterculiaceae such as the genera Theobroma e.g. the species Theobroma cacao [cacao]; Theaceae such as the genera Camellia e.g. the species Camellia sinensis) [tea].

Particular preferred plants are monocotyledonous or dicotyledonous plants selected from the group consisting of Asteraceae such as the genera Helianthus, Tagetes e.g. the species Helianthus annus [sunflower], Tagetes lucida, Tagetes erecta or Tagetes tenuifolia [Marigold]; Brassicaceae such as the genera Brassica, Arabadopsis e.g. the species Brassica napus, Brassica rapa ssp. [canola, oilseed rape, turnip rape] or Arabidopsis thaliana; Fabaceae such as the genera Glycine e.g. the species Glycine max [soybean] or Soja hispida; Linaceae such as the genera Linum e.g. the species Linum usitatissimum, [flax, linseed]; Poaceae such as the genera Hordeum, Secale, Avena, Sorghum, Oryza, Zea, Triticum e.g. the species Hordeum vulgare [barley], Secale cereale [rye], Avena sativa, Avena fatua, Avena byzantina, Avena fatua var. sativa, Avena hybrida [oat], Sorghum bicolor [Sorghum, millet], Oryza sativa, Oryza latifolia [rice], Zea mays [corn, maize], Triticum aestivum, Triticum durum, Triticum turgidum, Triticum hybernum, Triticum macha, Triticum sativum or Triticum vulgare [wheat, bread wheat, common wheat]; Solanaceae such as the genera Solanum, Lycopersicon e.g. the species Solanum tuberosum [potato], Lycopersicon esculentum, Lycopersicon lycopersicum, Lycopersicon pyriforme, Solanum integrifolium or Solanum lycopersicum [tomato]; Gramineae such as the genera Saccharum e.g. the species Saccharum officinarum or Helianthus annus [sunflower] or Chenopodiaceae such as the genera Beta, i.e. Beta vulgaris var. esculenta [sugar beet].

All above-mentioned organisms can be used as host organisms and/or can be used as donor organism.

The invention is not limited to specific nucleic acids, specific polypeptides, specific cell types, specific host cells, specific conditions or specific methods etc. as such, but may vary and numerous modifications and variations therein will be apparent to those skilled in the art. It is also to be understood that the terminology used herein is for the purpose of describing specific embodiments only and is not intended to be limiting.

In another embodiment, the invention relates to a (isolated) nucleic acid molecule, which comprises a nucleic acid molecule selected from the group consisting of:
(a) a nucleic acid molecule encoding a polypeptide shown in Table II, application no. 7, column 5 or 8, preferably shown in Table II B, application no. 7, column 8, or a homolog or a fragment thereof;
(b) a nucleic acid molecule shown in Table I, application no. 7, column 5 or 8, preferably shown in Table I B, application no. 7, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 7, preferably in column 8 of Table II B, application no. 7;
(d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 7, preferably shown in column 8 of Table I B, application no. 7, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 7;
(i) a nucleic acid molecule encoding a polypeptide having the activity represented by a respective protein as depicted in column 5 of Table II, application no. 7;
(j) a nucleic acid molecule comprising a polynucleotide which is obtained by amplifying a cDNA library or a genomic library using the primers in Table III, application no. 7, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 7.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II application no. 7 and confers a generation or an increase of the respective fine chemical in a non-human organism or a part thereof as compared to a corresponding wild-type non-human organism or part thereof.

In another embodiment, the invention relates to a (isolated) nucleic acid molecule, which comprises a nucleic acid molecule selected from the group consisting of:
(a) a nucleic acid molecule encoding a polypeptide shown in Table II, column 5 or 8, application no. 7, preferably shown in Table II A, application no. 7, in column 5 or in Table II A, application no. 7, column 8 or in Table II B, application no. 7, column 8, or a homolog or a fragment thereof;
(b) a nucleic acid molecule shown in Table I, application no. 7, column 5 or 8, preferably shown in Table I A, application no. 7, in column 5 or in Table I A, application no. 7, column 8 or in Table I B, application no. 7, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 7, preferably shown in Table II A, application no. 7, in column 5 or in Table II A, application no. 7, column 8 or in Table II B, application no. 7, column 8;
(d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 7, preferably shown in Table I A, application no. 7, in column 5 or in Table I A, application no. 7, column 8 or in Table I B column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 7;
(i) a nucleic acid molecule encoding a polypeptide having the activity represented by a respective protein as depicted in column 5 of Table II, application no. 7;
(j) a nucleic acid molecule comprising a polynucleotide which is obtained by amplifying a cDNA library or a genomic library using the primers in Table III, application no. 7, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt,500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto;
   whereby, preferably, the nucleic acid molecule according to (a), (b), (c), (d), (e), (f), (g), (h), (i), (j) or (k) distinguishes over the sequence depicted in Table I A and/or I B, application no. 7, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 7, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 7, column 5 or 8, or the coding region thereof, respectively. In a further embodiment the nucleic acid molecule does not encode the polypeptide sequence shown in Table II A and/or II B, application no. 7, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 7, column 5 or 8 and does not encode a protein of the sequence shown in Table II A and/or II B, application no. 7, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 7, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 7, column 5 or 8 but less than 100%, preferably less than 99.999%, 99.99%, 99.9%, 99%, 98%, 97%, 96% or 95% identical to the sequence shown in Table II A and/or II B, application no. 7, column 5 or 8.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 7.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II, application no. 7, and confers a generation or an increase of the respective fine chemical in a non-human organism or a part thereof as compared to a corresponding wild-type non-human organism or part thereof.

In one embodiment the invention relates to the aforementioned nucleic acid molecules, which can be isolated advantageously from yeast, fungi, viruses, algae, bacteria, for example from Charophyceae such as the genera Chara, Nitella e.g. the species Chara globularis, Chara vulgaris, Nitella flexilis, Chlorophyceae such as the genera Acrosiphonia, Spongomorpha, Urospora, Bryopsis, Pseudobryopsis, Trichosolen, Dichotomosiphon, Caulerpa, Rhipilia, Blastophysa, Avrainvillea, Chlorodesmis, Codium, Espera, Halicystis, Halimeda, Penicillus, Pseudocodium, Rhipiliopsis, Rhipocephalus, Tydemania, Udotea, Derbesia, Acrochaete, Aphanochaete, Bolbocoleon, Chaetobolus, Chaetonema, Chaetophora, Chlorotylium, Desmococcus, Draparnaldia, Draparnaldiopsis, Ectochaete, Endophyton, Entocladia, Epicladia, Internoretia, Microthamnion, Ochlochaete, Phaeophila, Pilinella, Pringsheimiella, Protoderma, Pseudendoclonium, Pseudodictyon, Pseudopringsheimia, Pseudulvella, Schizomeris, Stigeoclonium, Thamniochaete, Ulvella, Pilinia, Tellamia, Helicodictyon, Actidesmium, Ankyra, Characium, Codiolum, Sykidion, Keratococcus, Prototheca, Bracteacoccus Chlorococcum, Excentrosphaera, Hormidium, Oophila, Schroederia, Tetraedron, Trebouxia Chlorosarcinopsis, Gomphonitzschia, Coccomyxa, Dactylothece, DiÓgenes, Disporá, Gloeocystis, Mycanthococcus, Ourococcus, Coelastrum, Dicranochaete, Botryococcus, Dictyosphaerium, Dimorphococcus, Chlorochytrium, Kentrosphaera, Phyllobium, Gomontia, Hormotila, Euastropsis, Hydrodictyon, Pectodictyon, Pediastrum, Sorastrum, Tetrapedia, Acanthosphaera, Echinosphaerella, Echinosphaeridium, Errerella, Gloeoactinium, Golenkeniopsis Golenkinia, Micractinium, Ankistrodesmus, Chlorella, Chodatella, Closteriopsis, Cryocystis, Dactylococcus, Dematractum, Eremosphaera, Eutetramorus, Franceia, Glaucocystis, Gloeotaenium, Kirchneriella, Lagerheimiella, Monoraphidium, Nannochloris, Nephrochlamys, Nephrocytium, Oocystis, Oonephris, Pachycladon, Palmellococcus, Planktosphaeria, Polyedriopsis, Pseudoraciborskia, Quadrigula, Radiococcus, Rochiscia, Scotiella, Selanastrum, Thorakochloris, Treubaria, Trochiscia, Westella, Zoochlorella, Ostreobium, Phyllosiphon, Protosiphon, Rhodochytrium, Actinastrum, Coronastrum, Crucigenia, Dictymocystis, Enallax, Scenedesmus, Selenastrum, Tetradesmus, Tetrallantos, Tetrastrum, Chlorosarcina, Anadyomene, Valoniopsis, Ventricaria, Basicladia,

Chaetomorpha, Cladophora, LolaPithophoraRhizoclonium Chaetosphaeridium, Conochaete, Coleochaete, Oligochaetophora, Polychaetophora, Cylindrocapsa, Gongrosira, Protococcus, Acetabularia, Batophora, Bornetella, Dasycladus, Halicoryne, Neomeris, Elakatothrix, Raphidonema, Microspora, Bulbochaete, Oedocladium, Oedogonium, Prasiola, Rosenvingiella, Schizogonium, Apjohnia, Chamaedoris, Cladophoropsis, Siphonocladus, Spongocladia, Boergesenia, Boodlea, Cystodictyon, Dictyosphaeria, Ernodesmis, Microdictyon, Struvea, Valonia, Sphaeroplea, Malleochloris, Stylosphaeridium, Gloeococcus, Palmella, Palmodictyon, Palmophyllum, Pseudospherocystis, Sphaerocystis, Urococcus, Apiocystis, Chaetopeltis, Gemellicystis, Paulschulzia, Phacomyxa, Pseudotetraspora, Schizochlamys, Tetraspora, Cephaleuros, Ctenocladus, Epibolium, Leptosira, Trentepohlia, Diplochaete, Monostroma, Binuclearia, Geminella, Klebsormidium, Planetonema, Radiofilum, Stichococcus, Ulothrix, Uronema, Blidingia, Capsosiphon, Chloropelta, Enteromorpha, Percursaria, Ulva, Ulvaria, Brachiomonas, Carteria, Chlainomonas, Chlamydomonas, Chlamydonephris, Chlorangium, Chlorogonium, Cyanidium, Fortiella, Glenomonas, Gloeomonas, Hyalogonium, Lobomonas Polytoma, Pyramichlamys, Scourfieldia, Smithsonimonas, Sphaerellopsis, Sphenochloris, Spirogonium, Collodictyon, Dunaliella, Haematococcus, Stephanosphaera, Coccomonas, Dysmorphococcus, Phacotus, Pteromonas, Thoracomonas, Wislouchiella, Mascherina, Pyrobotrys, Spondylomorum, Eudorina, Gonium, Oltmannsiella, Pandorina, Platydorina, Pleodorina, Stephanoon, Volvox, Volvulina, Actinotaenium, Arthrodesmus, Bambusina Closterium, Cosmarium, Desmidium, Euastrum, Groenbladia, Hyalotheca, Micrasterias, Penium, Phymatodocis, Pleurotaenium, Sphaerozosma, Spinoclosterium, Spinocosmarium, Spondylosium, Staurastrum, Tetmemorus, Triploceras, Xanthidium, Cylindrocystis, Genicularia, Gonatozygon, Mesotaenium, Netrium, Roya, Spirotaenia, Cosmocladium, Debarya, Docidium, Euastridium, Hallasia, Mougeotia, Mougeotiopsis, Sirogonium, Spirogyra, Staurodesmus, Teilingia, Zygnema, Zygogonium, e.g. the species Caulerpa taxifolia, Prototheca wickerhamii, Ankistrodesmus falcatus, Chlorella ellipsoidea, Chlorella pyrenoidosa, Clorella sorokiniana, Chlorella vulgaris, Scenedesmus obliquus, Scenedesmus quadricauda, Selenastrum capricornutum, Selenastrum undecimnotata, Cladophora glomerata, Chlamydomonas eugametos, Chlamydomonas reinhardtii, Cyanidium caldarium, Dunaliella salina, Dunaliella tertiolecta, Euglena gracilis, Haematococcus pluvialis, Coniugatophyceae, Prasinophyceae Trebouxiophyceae, Ulvophyceae, Chlorodendraceae, Pedinomonadales, Halosphaeraceae, Pterospermataceae, Monomastigaceae, Pyramimonadaceae, Chlorodendraceae such as the genera Prasinocladus e.g. the species Prasinocladus ascus, Halosphaeraceae, Pedinomonadales, Pedinomonadaceae such as the genera Pedinomonas, Pterospermataceae such as the genera Pachysphaera, Pterosperma, Halosphaera, Pyramimonas, Bacillariophyceae, Chrysophyceae, Craspedophyceae, Euglenophyceae, Prymnesiophyceae, Phaeophyceae, Dinophyceae, Rhodophyceae, Xanthophyceae, Prasinophyceae such as the genera Nephroselmis, Prasinococcus, Scherffelia, Tetraselmis, Mantoniella, Ostreococcus e.g. the species Nephroselmis olivacea, Prasinococcus capsulatus, Scherffelia dubia, Tetraselmis chui, Tetraselmis suecica, Mantoniella squamata, Ostreococcus tauri, especially such as Acetobacter (subgen. Acetobacter) aceti; Acidithiobacillus ferrooxidans; Acinetobacter sp.; Actinobacillus sp; Aeromonas salmonicida; Agrobacterium tumefaciens; Aquifex aeolicus; Arcanobacterium pyogenes; Aster yellows phytoplasma; Bacillus sp.; Bifidobacterium sp.; Borrelia burgdorferi; Brevibacterium linens; Brucella melitensis; Buchnera sp.; Butyrivibrio fibrisolvens; Campylobacter jejuni; Caulobacter crescentus; Chlamydia sp.; Chlamydophila sp.; Chlorobium limicola; Citrobacter rodentium; Clostridium sp.; Comamonas testosteroni; Corynebacterium sp.; Coxiella burnetii; Deinococcus radiodurans; Dichelobacter nodosus; Edwardsiella ictaluri; Enterobacter sp.; Erysipelothrix rhusiopathiae; E. coli; Flavobacterium sp.; Francisella tularensis; Frankia sp. Cpl1; Fusobacterium nucleatum; Geobacillus stearothermophilus; Gluconobacter oxydans; Haemophilus sp.; Helicobacter pylori; Klebsiella pneumoniae; Lactobacillus sp.; Lactococcus lactis; Listeria sp.; Mannheimia haemolytica; Mesorhizobium loti; Methylophaga thalassica; Microcystis aeruginosa; Microscilla sp. PRE1; Moraxella sp. TA144; Mycobacterium sp.; Mycoplasma sp.; Neisseria sp.; Nitrosomonas sp.; Nostoc sp. PCC 7120; Novosphingobium aromaticivorans; Oenococcus oeni; Pantoea citrea; Pasteurella multocida; Pediococcus pentosaceus; Phormidium foveolarum; Phytoplasma sp.; Plectonema boryanum; Prevotella ruminicola; Propionibacterium sp.; Proteus vulgaris; Pseudomonas sp.; Ralstonia sp.; Rhizobium sp.; Rhodococcus equi; Rhodothermus marinus; Rickettsia sp.; Riemerella anatipestifer; Ruminococcus flavefaciens; Salmonella sp.; Selenomonas ruminantium; Serratia entomophila; Shigella sp.; Sinorhizobium meliloti; Staphylococcus sp.; Streptococcus sp.; Streptomyces sp.; Synechococcus sp.; Synechocystis sp. PCC 6803; Thermotoga maritima; Treponema sp.; Ureaplasma urealyticum; Vibrio cholerae; Vibrio parahaemolyticus; Xylella fastidiosa; Yersinia sp.; Zymomonas mobilis, preferably Salmonella sp. or E. coli, preferably from yeasts such as from the genera Saccharomyces, Pichia, Candida, Hansenula, Torulopsis or Schizosaccharomyces, or from plants, such as monocotyledonous or dicotyledonous plants, preferably selected from the group consisting of Anacardiaceae such as the genera Pistacia, Mangifera, Anacardium e.g. the species Pistacia vera [pistachios, Pistazie], Mangifer indica [Mango] or Anacardium occidentale [Cashew]; Asteraceae such as the genera Calendula, Carthamus, Centaurea, Cichorium, Cynara, Helianthus, Lactuca, Locusta, Tagetes, Valeriana e.g. the species Calendula officinalis [Marigold], Carthamus tinctorius [safflower], Centaurea cyanus [cornflower], Cichorium intybus [blue daisy], Cynara scolymus [Artichoke], Helianthus annus [sunflower], Lactuca sativa, Lactuca crispa, Lactuca esculenta, Lactuca scariola L. ssp. sativa, Lactuca scariola L. var. integrata, Lactuca scariola L. var. integrifolia, Lactuca sativa subsp. romana, Locusta communis, Valeriana locusta [lettuce], Tagetes lucida, Tagetes erecta or Tagetes tenuifolia [Marigold]; Apiaceae such as the genera Daucus e.g. the species Daucus carota [carrot]; Betulaceae such as the genera Corylus e.g. the species Corylus avellana or Corylus colurna [hazelnut]; Boraginaceae such as the genera Borago e.g. the species Borago officinalis [borage]; Brassicaceae such as the genera Brassica, Melanosinapis, Sinapis, Arabadopsis e.g. the species Brassica napus, Brassica rapa ssp. [canola, oilseed rape, turnip rape], Sinapis arvensis Brassica juncea, Brassica juncea var. juncea, Brassica juncea var. crispifolia, Brassica juncea var. foliosa, Brassica nigra, Brassica sinapioides, Melanosinapis communis [mustard], Brassica oleracea [fodder beet] or Arabidopsis thaliana; Bromeliaceae such as the genera Anana, Bromelia e.g. the species Anana comosus, Ananas ananas or Bromelia comosa [pineapple]; Caricaceae such as the genera Carica e.g. the species Carica papaya [papaya]; Cannabaceae such as the genera Cannabis e.g. the species Cannabis sative [hemp], Convolvulaceae such as the genera Ipomea, Convolvulus e.g. the species Ipomoea batatus, Ipomoea pandurata, Convolvulus batatas, Convolvulus tiliaceus, Ipomoea fastigiata, Ipomoea tiliacea, Ipomoea triloba or Convolvulus panduratus [sweet potato, Man of the Earth, wild potato], Chenopodiaceae such as the genera Beta, i.e. the species Beta vulgaris, Beta vulgaris var. altissima, Beta vulgaris var. Vulgaris, Beta maritima, Beta vulgaris var. perennis, Beta vulgaris var. conditiva or Beta vulgaris var. esculenta [sugar beet]; Cucurbitaceae such as the genera Cucubita e.g. the species Cucurbita maxima, Cucurbita mixta, Cucurbita pepo or Cucurbita moschata [pumpkin, squash]; Elaeagnaceae such as the genera Elaeagnus e.g. the species Olea europaea [olive]; Ericaceae such as the genera Kalmia e.g. the species Kalmia latifolia, Kalmia angustifolia, Kalmia microphylla, Kalmia polifolia, Kalmia occidentalis, Cistus chamaerhodendros or Kalmia lucida [American laurel, broad-leafed laurel, calico bush, spoon wood, sheep laurel, alpine laurel, bog laurel, western bog-laurel, swamp-laurel]; Euphorbiaceae such as the genera Manihot, Janipha, Jatropha, Ricinus e.g. the species Manihot utilissima, Janipha manihot, Jatropha manihot, Manihot aipil, Manihot dulcis, Manihot manihot, Manihot melanobasis, Manihot esculenta [manihot, arrowroot, tapioca, cassava] or Ricinus communis [castor bean, Castor Oil Bush, Castor Oil Plant, Palma Christi, Wonder Tree]; Fabaceae such as the genera Pisum, Albizia, Cathormion, Feuillea, Inga, Pithecolobium, Acacia, Mimosa, Medicajo, Glycine, Dolichos, Phaseolus, Soja e.g. the species Pisum sativum, Pisum arvense, Pisum humile [pea], Albizia berteriana, Albizia julibrissin, Albizia lebbeck, Acacia berteriana, Acacia littoralis, Albizia berteriana, Albizzia berteriana, Cathormion berteriana, Feuillea berteriana, Inga fragrans, Pithecellobium berterianum, Pithecellobium fragrans, Pithecolobium berterianum, Pseudalbizzia berteriana, Acacia julibrissin, Acacia nemu, Albizia nemu, Feuilleea julibrissin, Mimosa julibrissin, Mimosa speciosa, Sericanrda julibrissin, Acacia lebbeck, Acacia macrophylla, Albizia lebbek, Feuilleea lebbeck, Mimosa lebbeck, Mimosa speciosa [bastard logwood, silk tree, East Indian Walnut], Medicago sativa, Medicago falcata, Medicago varia [alfalfa], Glycine max [soybean], Dolichos soja, Glycine gracilis, Glycine hispida, Phaseolus max or Soja hispida; Geraniaceae such as the genera Pelargonium, Cocos, Oleum e.g. the species Cocos nucifera, Pelargonium grossularioides or Oleum cocois [coconut]; Gramineae such as the genera Saccharum e.g. the species Saccharum officinarum; Juglandaceae such as the genera Juglans, Wallia e.g. the species Juglans regia, Juglans ailanthifolia, Juglans sieboldiana, Juglans cinerea, Wallia cinerea, Juglans bixbyi, Juglans californica, Juglans hindsii, Juglans intermedia, Juglans jamaicensis, Juglans major, Juglans microcarpa, Juglans nigra or Wallia nigra [walnut, black walnut, common walnut, persian walnut, white walnut, butternut, black walnut]; Lauraceae such as the genera Persea, Laurus e.g. the species laurel Laurus nobilis [bay, laurel, bay laurel, sweet bay], Persea americana Persea americana, Persea gratissima or Persea persea [avocado]; Leguminosae such as the genera Arachis e.g. the species Arachis hypogaea [peanut]; Linaceae such as the genera Linum, Adenolinum e.g. the species Linum usitatissimum, Linum humile, Linum austriacum, Linum bienne, Linum angustifolium, Linum catharticum, Linum flavum, Linum grandiflorum, Adenolinum grandiflorum, Linum lewisii, Linum narbonense, Linum perenne, Linum perenne var. lewisii, Linum pratense or Linum trigynum [flax, linseed]; Lythrarieae such as the genera Punica e.g. the species Punica granatum [pomegranate]; Malvaceae such as the genera Gossypium e.g. the species Gossypium hirsutum, Gossypium arboreum, Gossypium barbadense, Gossypium herbaceum or Gossypium thurberi [cotton]; Musaceae such as the genera Musa e.g. the species Musa nana, Musa acuminate, Musa paradisiaca, Musa spp. [banana]; Onagraceae such as the genera Camissonia, Oenothera e.g. the species Oenothera biennis or Camissonia brevipes [primrose, evening primrose]; Palmae such as the genera Elacis e.g. the species Elaeis guineensis [oil plam]; Papaveraceae such as the genera Papaver e.g. the species Papaver orientale, Papaver rhoeas, Papaver dubium [poppy, oriental poppy, corn poppy, field poppy, shirley poppies, field poppy, longheaded poppy, long-pod poppy]; Pedaliaceae such as the genera Sesamum e.g. the species Sesamum indicum [sesame]; Piperaceae such as the genera Piper, Artanthe, Peperomia, Steffensia e.g. the species Piper aduncum, Piper amalago, Piper angustifolium, Piper auritum, Piper betel, Piper cubeba, Piper longum, Piper nigrum, Piper retrofractum, Artanthe adunca, Artanthe elongata, Peperomia elongata, Piper elongatum, Steffensia elongata. [Cayenne pepper, wild pepper]; Poaceae such as the genera Hordeum, Secale, Avena, Sorghum, Andropogon, Holcus, Panicum, Oryza, Zea, Triticum e.g. the species Hordeum vulgare, Hordeum jubaturn, Hordeum murinum, Hordeum secalinum, Hordeum distichon Hordeum aegiceras, Hordeum hexastichon, Hordeum hexastichum, Hordeum irregulare, Hordeum sativum, Hordeum secalinum [barley, pearl barley, foxtail barley, wall barley, meadow barley], Secale cereale [rye], Avena sativa, Avena fatua, Avena byzantina, Avena fatua var. sativa, Avena hybrida [oat], Sorghum bicolor, Sorghum halepense, Sorghum saccharatum, Sorghum vulgare, Andropogon drummondii, Holcus bicolor, Holcus sorghum, Sorghum aethiopicum, Sorghum arundinaceum, Sorghum caffrorum, Sorghum cernuum, Sorghum dochna, Sorghum drummondii, Sorghum durra, Sorghum guineense, Sorghum lanceolatum, Sorghum nervosum, Sorghum saccharatum, Sorghum subglabrescens, Sorghum verticilliflorum, Sorghum vulgare, Holcus halepensis, Sorghum miliaceum millet, Panicum militaceum [Sorghum, millet], Oryza sativa, Oryza latifolia [rice], Zea mays [corn, maize] Triticum aestivum, Triticum durum, Triticum turgidum, Triticum hybernum, Triticum macha, Triticum sativum or Triticum vulgare [wheat, bread wheat, common wheat], Proteaceae such as the genera Macadamia e.g. the species Macadamia intergrifolia [macadamia]; Rubiaceae such as the genera Coffea e.g. the species Cofea spp., Coffea arabica, Coffea canephora or Coffea liberica [coffee]; Scrophulariaceae such as the genera Verbascum e.g. the species Verbascum blattaria, Verbascum chaixii, Verbascum densiflorum, Verbascum lagurus, Verbascum longifolium, Verbascum lychnitis, Verbascum nigrum, Verbascum olympicum, Verbascum phlomoides, Verbascum phoenicum, Verbascum pulverulentum or Verbascum thapsus [mullein, white moth mullein, nettle-leaved mullein, dense-flowered mullein, silver mullein, long-leaved mullein, white mullein, dark mullein, greek mullein, orange mullein, purple mullein, hoary mullein, great mullein]; Solanaceae such as the genera Capsicum, Nicotiana, Solanum, Lycopersicon e.g. the species Capsicum annuum, Capsicum annuum var. glabriusculum, Capsicum frutescens [pepper], Capsicum annuum [paprika], Nicotiana tabacum, Nicotiana alata, Nicotiana attenuata, Nicotiana glauca, Nicotiana langsdorffii, Nicotiana obtusifolia, Nicotiana quadrivalvis, Nicotiana repanda, Nicotiana rustica, Nicotiana sylvestris [tobacco], Solanum tuberosum [potato], Solanum melongena [egg-plant] (Lycopersicon esculentum, Lycopersicon lycopersicum, Lycopersicon pyriforme, Solanum integrifolium or Solanum lycopersicum [tomato]; Sterculiaceae such as the genera Theobroma e.g. the species Theobroma cacao [cacao]; Theaceae such as the genera Camellia e.g. the species Camellia sinensis) [tea]; especially A. thaliana, maize, wheat, rye, oat, triticale, rice, barley, soybean, peanut, cotton, borage, sunflower, sugar cane, sugar beet, linseed, primrose, rapeseed, canola and turnip rape, manihot, pepper, sunflower, tagetes, solanaceous plant such as potato, tobacco, eggplant and tomato, Vicia species, pea, alfalfa, bushy plants such as coffee, cacao, tea, Salix species, trees such as oil palm, coconut, perennial grass, such as ryegrass and fescue, and forage crops, such as alfalfa and clover and from spruce, pine or fir for example.

More preferably said nucleic acid molecules can be isolated from S. cerevisiae, E. coli, Azotobacter vinelandii, Thermus thermophilus or i Synechocystis sp. or plants, preferably Arabidopsis thaliana, Brassica napus, Glycine max, Zea mays, Gossypium or Oryza sativa.

The FCRPs of the present invention are preferably produced by recombinant DNA techniques. For example, a nucleic acid molecule encoding the protein is cloned into an expression vector, for example into a binary vector, the expression vector is introduced into a host cell, for example into cells of the Arabidopsis thaliana wild type NASC N906 or any other plant cell as described above and/or below and/or in the examples, and the FCRP is expressed in said host cell.

In an embodiment of the present invention, the respective FCRP is localized after expression as indicated in column 6 of Table II, e.g. non-targeted, mitochondric or plastidic.

In an embodiment of the present invention the FCRP is preferably produced in an compartment of the cell, e.g. in an organelle, for example in plastids or mitochondria. Ways of introducing nucleic acids into organelles, for example into plastids or mitochondria, and producing proteins in this compartment are known to the person skilled in the art.

The respective nucleic acid sequence according to the invention mentioned above is advantageously functionally joined to a nucleic acid sequence encoding a transit peptide, in such a manner that a preprotein is translated, which is able to direct the polypeptide to the organelle such as to plastids or mitochondria. In another preferred embodiment the nucleic acids according to the invention mentioned above is advantageously functionally joined to a promoter region functional in plastids or mitochondria like for example the RNA operon promoter fused to the 5s UTR of the rbcL gene and in another preferred embodiment joined to a plastome sequences homologous to the integration sites. Example for useful integration sites are the trnV-rps12/7 (Skidar et al., Plant Cell Rep. 18, 20 (1998), and other reports), thr rbvL-aacD site (Svab et al., Proc. Natl. Acad. Sci. USA 90, 913 (1993)), the trnl-trnA site (De Cosa et al., Nat. Biotech. 19, 71 (2001)), the rps7-ndhB site (Hou et al., Transgenic Res. 12, 111 (2003)) and the ndhF-trnL site Zhang et al., Plant Physiol. 127, 131 (2001c)).

The nucleic acid sequence coding for the transit peptide is advantageously derived from a nucleic acid sequence encoding a protein finally resided in the plastids or mitochondria and is stemming from an organism selected from the group consisting of the genera Acetabularia, Arabidopsis, Brassica, Capsicum, Chlamydomonas, Cucurbita, Daucus, Dunaliella, Euglena, Flaveria, Glycine, Helianthus, Hordeum, Lactua, Lemna, Lolium, Lycopersion, Malus, Meticago, Mesembryanthemum, Nicotiana, Oenotherea, Oryza, PetOnia, Phaseolus, Physcomitrella, Pinus, Pisum, Pyrus, Raphanus, Saccharum, Silene, Sinapis, Solanum, Spinacea, Stevia, Synechococcus,Triticum and Zea.

Preferably the plastidal transit peptide is derived from the nucleic acid sequence encoding a protein selected from the group consisting of ribulose bisphosphate carboxylase/oxygenase, 5-enolpyruvyl-shikimate-3-phosphate synthase, acetolactate synthase, chloroplast ribosomal protein CS17, Cs protein, ferredoxin, plastocyanin, ribulose bisphosphate carboxylase activase, tryptophan synthase, acyl carrier protein, plastid chaperonin-60, cytochrome c552, 22-kDA heat shock protein, 33-kDa Oxygen-evolving enhancer protein 1, ATP synthase γ subunit, ATP synthase δ subunit, chlorophyll-a/b-binding proteinII-1, Oxygen-evolving enhancer protein 2, Oxygen-evolving enhancer protein 3, photosystem I: P21, photosystem I: P28, photosystem I: P30, photosystem I: P35, photosystem I: P37, glycerol-3-phosphate acyltransferases, chlorophyll a/b binding protein, CAB2 protein, hydroxymethyl-bilane synthase, pyruvate-orthophosphate dikinase, CAB3 protein, plastid ferritin, ferritin, early light-inducible protein, glutamate-1-semialdehyde aminotransferase, protochlorophyllide reductase, starch-granule-bound amylase synthase, light-harvesting chlorophyll a/b-binding protein of photosystem II, major pollen allergen Lol p 5a, plastid CIpB ATP-dependent protease, superoxide dismutase, ferredoxin NADP oxidoreductase, 28-kDa ribonucleoprotein, 31-kDa ribonucleoprotein, 33-kDa ribonucleoprotein, acetolactate synthase, ATP synthase CFO subunit 1, ATP synthase CFO subunit 2, ATP synthase CFO subunit 3, ATP synthase CFO subunit 4, cytochrome f, ADP-glucose pyrophosphorylase, glutamine synthase, glutamine synthase 2, carbonic anhydrase, GapA protein, heatshock-protein hsp21, phosphate translocator, plastid CIpA ATP-dependent protease, plastid ribosomal protein CL24, plastid ribosomal protein CL9, plastid ribosomal protein PsCL18, plastid ribosomal protein PsCL25, DAHP synthase, starch phosphorylase, root acyl carrier protein II, betaine-aldehyde dehydrogenase, GapB protein, glutamine synthetase 2, phosphoribulokinase, nitrite reductase, ribosomal protein L12, ribosomal protein L13, ribosomal protein L21, ribosomal protein L35, ribosomal protein L40, triose phosphate-3-phosphoglyerate-phosphate translocator, ferredoxin-dependent glutamate synthase, glyceraldehyde-3-phosphate dehydrogenase, NADP-dependent malic enzyme and NADP-malate dehydrogenase.

In an embodiment the plastome sequences are preferential derived from the plastome of the target organisms themselves and are advantegously derived from one of the following intergration sites: trnV-rps12/7 (Skidar et al., Plant Cell Rep. 18, 20 (1998) and other reports), rbvLaacD (Svab et al., Proc. Natl. Acad. Sci. USA 90, 913 (1993)), trnl-trnA (De Cosa et al., Nat. Biotech. 19, 71 (2001)), rps7-ndhB (Hou et al., Transgenic Res. 12, 111 (2003)) or ndhF-trnL site Zhang et al., Plant Physiol. 127, 131 (2001c)).

Advantageously the mitochondrial transit peptide is derived from the nucleic acid sequence encoding a protein selected from the group consisting of 22 kDA heat shock protein; 70 kDA heat shock protein; 83 kDA heat shock protein; 40S ribosomal protein S19; 50S ribosomal protein L15; ribosomal protein L29; 22 kDA PSST protein of complex I; 2-oxoacid dehydrogenase family protein; 2-oxoglutarate/malate translocator; 3methyl-2-oxobutanoate hydroxymethyltransferase; 3-Methylcrotonyl-coenzyme A carboxylase (MCCase); 7,8 -Dihydropteroate synthase (DHPS)/6-hydroxymethyl-7,8-dihydropterine pyrophosphokinase (HPPK); aconitate hydratase; acyl carrier protein (ACP); ADP/ATP translocase; alanyl-tRNA synthetase; alcohol dehydrogenase (ADH); alternative oxidase (AOX); aminoacyl-tRNA ligase; asparate aminotransferase; ATP synthase alpha subunit; ATP synthase beta subunit; ATP synthase delta subunit; ATP synthase epsilon subunit; ATP synthase gamma subunit; ATP-dependent Clp protease-proteolytic subunit; Chaperonin 60 - CPN60; Chaperonin 60 (2) CPN 60-2; Chaperonin 60(1)-CPN60-1; citrate synthase; cytochrome b-c1 complex subunit Rieske FeS Protein; cytochrome c reductase-processing peptidase subunit II; dihydrolipoamide S-acetyltransferase; farnesyl-diphosphate synthase 1; formate dehydrogenase; fumarate hydratase; gamma carbonic anhydrase protein (gammaCA); gamma carbonic anhydrase-like protein 1 (gammaCAL1); gamma carbonic anhydrase-like protein 2 (gammaCAL2); gamma-aminobutyric acid transaminase (GABA-T); glutathione reductase (GR); glycine decarboxylase subunit H; glycine decarboxylase subunit L; glycine decarboxylase subunit P; glycine decarboxylase subunit T; isovaleryl-CoA dehydrogenase (IVD); lipoamide dehydrogenase; malate oxidoreductase; manganese superoxide dismutase (Mn)SOD; methylmalonate-semialdehyde dehydrogenase; mitochondrial-processing peptidase beta subunit (MPP); mitochondrial processing peptidase subunit alpha (MPP); monodehydroascorbate reductase (MDHAR); NAD dependent isocitrate dehydrogenase; NAD dependent malate dehydrogenase; NAD-dependent malic enzyme; NAD-dependent malic enzyme 59 kDa isoform; NAD-dependent malic enzyme 62 kDa isoform; NAD-dependent malic enzyme 65 kDa isoform; NADH ubiquinone oxidoreductase 29 kDa subunit; NADH-ubiquinone oxidoreductase 18 kDa subunit; NADH-ubiquinone oxidoreductase 20 kDa subunit; NADH-ubiquinone oxidoreductase 23 kDa subunit; NADHubiquinone oxidoreductase 75 kDa subunit; NADP dependent isocitrate dehydrogenase; NADP dependent malate dehydrogenase; nucleoside diphosphate kinase; nucleoside diphosphate kinase III; o-acetylserine (thiol) lyase (OAS-TL); propionyl-CoA carboxylase; protoporphyrinogen IX oxidase; pyruvate dehydrogenase E1 component subunit alpha; serine acetyltransferase (SAT); serine hydroxymethyltransferase; succinate dehydrogenase (SDH); succinic semialdehyde dehydrogenase (SSADH); succinyl-CoA ligase (GDP-forming) alpha-chain; succinyl-CoA ligase [GDP-forming] subunit beta; thiosulfate sulfurtransferase; threonyl-tRNA synthetase; trans-2-enoyl-CoA reductase; translocase inner membrane (TIM); translocase outer membrane (TOM); tRNA synthetase class I and ubiquinol cytochrome C oxidoreductase complex.

Further transit peptides and details thereto have been described above.

In another embodiment of the present invention the FCRP is produced without further targeting signal (e.g. as mentioned herein), e.g. in the cytoplasm of the cell. Ways of producing proteins without artificial targeting are known to the person skilled in the art.

The nucleic acid sequences used in the process are advantageously introduced in a nucleic acid construct, preferably an expression cassette, which makes possible the expression of the nucleic acid molecules in a non-human organism, advantageously a plant or a microorganism such as an algae, advantageously non-targeted or in an organelle, like plastids or mitochondria , respectively, of those non-human organisms.

Accordingly, the invention also relates to a nucleic acid construct, preferably to an expression construct, comprising the nucleic acid molecule of the present invention functionally linked to one or more regulatory elements or signals.

As described herein, the nucleic acid construct can also comprise further genes, which are to be introduced into the non-human organisms or cells. It is possible and advantageous to introduce into, and express in, the host organisms regulatory genes such as genes for inductors, repressors or enzymes, which, owing to their enzymatic activity, engage in the regulation of one or more genes of the respective biosynthetic pathway. These genes can be of heterologous or homologous origin. Moreover, further biosynthesis genes may advantageously be present, or else these genes may be located on one or more further nucleic acid constructs. Genes, which are advantageously employed as further biosynthesis genes are genes of the fatty acid metabolism, amino acid metabolism, of glycolysis, of the tricarboxylic acid metabolism or their combinations. As described herein, regulator sequences or factors can have a positive effect on preferably the gene expression of the genes introduced, thus increasing it. Thus, an enhancement of the regulator elements may advantageously take place at the transcriptional level by using strong transcription signals such as promoters and/or enhancers. In addition, however, an enhancement of translation is also possible, for example by increasing mRNA stability or by inserting a translation enhancer sequence.

In principle, the nucleic acid construct can comprise the herein described regulator sequences and further sequences relevant for the expression of the comprised genes. Thus, the nucleic acid construct of the invention can be used as expression cassette and thus can be used directly for introduction into the plant, or else they may be introduced into a vector. Accordingly in one embodiment the nucleic acid construct is an expression cassette comprising a microorganism promoter or a microorganism terminator or both, beneath the nucleic acid molecule according to the invention. In another embodiment the expression cassette encompasses a plant promoter or a plant terminator or both, beneath the nucleic acid molecule according to the invention. In another embodiment the expression cassette encompasses sequences for transcription by organelle RNA polymerases, beneath the nucleic acid molecule according to the invention. In another embodiment an expression cassette encompasses a nucleic acid molecule encoding for a transit peptide, beneath the gene according to the invention.

In a preferred embodiment a nucleic acid construct, for example an expression cassette, comprises upstream, i.e. at the 5′ end of the encoding sequence, a promoter and downstream, i.e. at the 3′ end, a polyadenylation signal and optionally other regulatory elements which are operably linked to the intervening encoding sequence with one of the nucleic acids as depicted in the respective line in Table I, application no. 7, column 5 or 8, preferably the coding region thereof, or a homolog or a fragment thereof. By an operable linkage is meant the sequential arrangement of promoter, encoding sequence, terminator, optionally other regulatory elements and optionally targeting sequences in such a way that each of the regulatory elements/targeting sequence can fulfill its function in the expression of the encoding sequence in due manner. In one embodiment the sequences preferred for operable linkage are targeting sequences for ensuring subcellular localization in organelles, like plastids or mitochondria. However, targeting sequences for ensuring subcellular localization in the endoplasmic reticulum (=ER), in the nucleus, in oil corpuscles or other compartments may also be employed as well as translation promoters such as the 5′ lead sequence in tobacco mosaic virus (Gallie et al., Nucl. Acids Res. 15, 8693 (1987)).

As an example, a nucleic acid construct, like an expression cassette may, for example, contain a constitutive promoter or a tissue-specific promoter (preferably the USP or napin promoter), the gene to be expressed and a ER retention signal. For the ER retention signal the KDEL amino acid sequence (lysine, aspartic acid, glutamic acid, leucine) or the KKX amino acid sequence (lysine-lysine-X-stop, wherein X means every other known amino acid) is preferably employed.

In an embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 7, column 5 or 8, preferably shown in Table II A, application no. 7, column 5, or in Table II A, application no. 7, column 8, or in Table II B, application no. 7, column 8, or a homolog or a fragment thereof;
   (b) a nucleic acid molecule shown in Table I, application no. 7, column 5 or 8, preferably shown in Table I A, application no. 7, column 5, or in Table I A, application no. 7, column 8, or in Table I B, application no. 7, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 7, preferably shown in Table II A, application no. 7, column 5, or in Table II A, application no. 7, column 8, or in Table II B, application no. 7, column 8;
   (d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 7, preferably shown in Table I A, application no. 7, column 5, or in Table I A, application no. 7, column 8, or in Table I B, application no. 7, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 7;
   (i) a nucleic acid molecule encoding a polypeptide having the activity represented by a respective protein as depicted in column 5 of Table II, application no. 7;
   (j) a nucleic acid molecule comprising a polynucleotide which is obtained by amplifying a cDNA library or a genomic library using the primers in Table III, application no. 7, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto.
      and, optionally
3) nucleic acid molecule encoding a transit peptide, preferably encoding a plastidal transit peptide or a mitochondrial transit peptide;
   which are operable linked.
   (All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 7.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 7 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 7, in the same line as of the respective nucleic acid molecule according to 2) “plastidic” is depicted, or encoding a mitochondrial transit peptide, especially in case in column 6 of Table I, application no. 7, in the same line as of the respective nucleic acid molecule according to 2) “mitochondric” is depicted.

In another embodiment thereof the expression cassette does not comprise 3) a nucleic acid molecule encoding a transit peptide, especially in case in column 6 of Table I, application no. 7, in the same line as of the respective nucleic acid molecule according to 2) “non-targeted” is depicted.

In a further embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 7, column 5 or 8, preferably shown in Table II A, application no. 7, column 5, or in Table II A, application no. 7, column 8, or in Table II B, application no. 7, column 8, or a homolog or a fragment thereof;
   (b) a nucleic acid molecule shown in Table I, application no. 7, column 5 or 8, preferably shown in Table I A, application no. 7, column 5, or in Table I A, application no. 7, column 8, or in Table I B, application no. 7, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 7, preferably shown in Table II A, application no. 7, column 5, or in Table II A, application no. 7, column 8, or in Table II B, application no. 7, column 8;
   (d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 7, preferably shown in Table I A, application no. 7, column 5, or in Table I A, application no. 7, column 8,or in Table I B, application no. 7, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 7;
   (i) a nucleic acid molecule encoding a polypeptide having the activity represented by a respective protein as depicted in column 5 of Table II, application no. 7;
   (j) a nucleic acid molecule comprising a polynucleotide which is obtained by amplifying a cDNA library or a genomic library using the primers in Table III, application no. 7, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, optionally
3) nucleic acid molecule encoding a transit peptide, preferably encoding a plastidal transit peptide or a mitochondrial transit peptide;
   which are operable linked and
   whereby, preferably, the nucleic acid molecule according to 2 (a), (b), (c), (d), (e), (f), (g), (h), (i), (j) or (k) distinguishes over the sequence depicted in Table I A and/or I B, application no. 7, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention according to 2) does not consist of the sequence shown in Table I A and/or I B, application no. 7, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 7, column 5 or 8, or the coding region thereof, respectively. In a further embodiment the nucleic acid molecule does not encode the polypeptide sequence shown in Table II A and/or II B, application no. 7, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 7, column 5 or 8 and does not encode a protein of the sequence shown in Table II A and/or II B, application no. 7, column 5 or 8. Accordingly, in one embodiment, the protein encoded by a sequence of a nucleic acid according to 2(a), (b), (c), (d), (e), (f), (g), (h), (i), (j) or (k) does not consist of the sequence shown in Table I A and/or I B, application no. 7, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 7, column 5 or 8 but less than 100%, 99.999%, 99.99%, 99.9%, 99%, 98%, 97%, 96% or 95% identical to the sequence shown in Table II A and/or II B, application no. 7, column 5 or 8.
   (All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 7.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 7 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 7, in the same line as of the respective nucleic acid molecule according to 2) “plastidic” is depicted, or encoding a mitochondrial transit peptide, especially in case in column 6 of Table I, application no. 7, in the same line as of the respective nucleic acid molecule according to 2) “mitochondric” is depicted.

In another embodiment thereof the expression cassette does not comprise 3) a nucleic acid molecule encoding a transit peptide, especially in case in column 6 of Table I, application no. 7, in the same line as of the respective nucleic acid molecule according to 2) “non-targeted” is depicted.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 15532, or a homolog or fragment thereof, or a homolog thereof being depicted in Table I, application no. 7, column 8, in the same line as SEQ ID NO. 15532, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 15532 is depicted, plastidic is mentioned, or a mitochondrial transit peptide in case in Table I, application no. 7, in column 6 in the same line as SEQ ID NO. 15532 is depicted, mitochondric is mentioned; which is operable linked, too.

In another embodiment thereof the expression cassette does not comprise an additional transit peptide in case in Table I, application no. 7, in column 6 in the same line as SEQ ID NO. 15532 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 16883, or a homolog or fragment thereof, or a homolog thereof being depicted in Table I, application no. 7, column 8, in the same line as SEQ ID NO. 16883, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 16883 is depicted, plastidic is mentioned, or a mitochondrial transit peptide in case in Table I, application no. 7, in column 6 in the same line as SEQ ID NO. 16883 is depicted, mitochondric is mentioned; which is operable linked, too.

In another embodiment thereof the expression cassette does not comprise an additional transit peptide in case in Table I, application no. 7, in column 6 in the same line as SEQ ID NO. 16883 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17901, or a homolog or fragment thereof, or a homolog thereof being depicted in Table I, application no. 7, column 8, in the same line as SEQ ID NO. 17901, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 17901 is depicted, plastidic is mentioned, or a mitochondrial transit peptide in case in Table I, application no. 7, in column 6 in the same line as SEQ ID NO. 17901 is depicted, mitochondric is mentioned; which is operable linked, too.

In another embodiment thereof the expression cassette does not comprise an additional transit peptide in case in Table I, application no. 7, in column 6 in the same line as SEQ ID NO. 17901 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 18070, or a homolog or fragment thereof, or a homolog thereof being depicted in Table I, application no. 7, column 8, in the same line as SEQ ID NO. 18070, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 18070 is depicted, plastidic is mentioned, or a mitochondrial transit peptide in case in Table I, application no. 7, in column 6 in the same line as SEQ ID NO. 18070 is depicted, mitochondric is mentioned; which is operable linked, too.

In another embodiment thereof the expression cassette does not comprise an additional transit peptide in case in Table I, application no. 7, in column 6 in the same line as SEQ ID NO. 18070 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 18235, or a homolog or fragment thereof, or a homolog thereof being depicted in Table I, application no. 7, column 8, in the same line as SEQ ID NO. 18235, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 18235 is depicted, plastidic is mentioned, or a mitochondrial transit peptide in case in Table I, application no. 7, in column 6 in the same line as SEQ ID NO. 18235 is depicted, mitochondric is mentioned; which is operable linked, too.

In another embodiment thereof the expression cassette does not comprise an additional transit peptide in case in Table I, application no. 7, in column 6 in the same line as SEQ ID NO. 18235 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 18869, or a homolog or fragment thereof, or a homolog thereof being depicted in Table I, application no. 7, column 8, in the same line as SEQ ID NO. 18869, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 18869 is depicted, plastidic is mentioned, or a mitochondrial transit peptide in case in Table I, application no. 7, in column 6 in the same line as SEQ ID NO. 18869 is depicted, mitochondric is mentioned; which is operable linked, too.

In another embodiment thereof the expression cassette does not comprise an additional transit peptide in case in Table I, application no. 7, in column 6 in the same line as SEQ ID NO. 18869 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 19419, or a homolog or fragment thereof, or a homolog thereof being depicted in Table I, application no. 7, column 8, in the same line as SEQ ID NO. 19419, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 19419 is depicted, plastidic is mentioned, or a mitochondrial transit peptide in case in Table I, application no. 7, in column 6 in the same line as SEQ ID NO. 19419 is depicted, mitochondric is mentioned; which is operable linked, too.

In another embodiment thereof the expression cassette does not comprise an additional transit peptide in case in Table I, application no. 7, in column 6 in the same line as SEQ ID NO. 19419 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 108727, or a homolog or fragment thereof, or a homolog thereof being depicted in Table I, application no. 7, column 8, in the same line as SEQ ID NO. 108727, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 108727 is depicted, plastidic is mentioned, or a mitochondrial transit peptide in case in Table I, application no. 7, in column 6 in the same line as SEQ ID NO. 108727 is depicted, mitochondric is mentioned; which is operable linked, too.

In another embodiment thereof the expression cassette does not comprise an additional transit peptide in case in Table I, application no. 7, in column 6 in the same line as SEQ ID NO. 108727 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 21159, or a homolog or fragment thereof, or a homolog thereof being depicted in Table I, application no. 7, column 8, in the same line as SEQ ID NO. 21159, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 21159 is depicted, plastidic is mentioned, or a mitochondrial transit peptide in case in Table I, application no. 7, in column 6 in the same line as SEQ ID NO. 21159 is depicted, mitochondric is mentioned; which is operable linked, too.

In another embodiment thereof the expression cassette does not comprise an additional transit peptide in case in Table I, application no. 7, in column 6 in the same line as SEQ ID NO. 21159 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 21497, or a homolog or fragment thereof, or a homolog thereof being depicted in Table I, application no. 7, column 8, in the same line as SEQ ID NO. 21497, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 21497 is depicted, plastidic is mentioned, or a mitochondrial transit peptide in case in Table I, application no. 7, in column 6 in the same line as SEQ ID NO. 21497 is depicted, mitochondric is mentioned; which is operable linked, too.

In another embodiment thereof the expression cassette does not comprise an additional transit peptide in case in Table I, application no. 7, in column 6 in the same line as SEQ ID NO. 21497 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 21902, or a homolog or fragment thereof, or a homolog thereof being depicted in Table I, application no. 7, column 8, in the same line as SEQ ID NO. 21902, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 21902 is depicted, plastidic is mentioned, or a mitochondrial transit peptide in case in Table I, application no. 7, in column 6 in the same line as SEQ ID NO. 21902 is depicted, mitochondric is mentioned; which is operable linked, too.

In another embodiment thereof the expression cassette does not comprise an additional transit peptide in case in Table I, application no. 7, in column 6 in the same line as SEQ ID NO. 21902 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 22015, or a homolog or fragment thereof, or a homolog thereof being depicted in Table I, application no. 7, column 8, in the same line as SEQ ID NO. 22015, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 22015 is depicted, plastidic is mentioned, or a mitochondrial transit peptide in case in Table I, application no. 7, in column 6 in the same line as SEQ ID NO. 22015 is depicted, mitochondric is mentioned; which is operable linked, too.

In another embodiment thereof the expression cassette does not comprise an additional transit peptide in case in Table I, application no. 7, in column 6 in the same line as SEQ ID NO. 22015 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 22249, or a homolog or fragment thereof, or a homolog thereof being depicted in Table I, application no. 7, column 8, in the same line as SEQ ID NO. 22249, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 22249 is depicted, plastidic is mentioned, or a mitochondrial transit peptide in case in Table I, application no. 7, in column 6 in the same line as SEQ ID NO. 22249 is depicted, mitochondric is mentioned; which is operable linked, too.

In another embodiment thereof the expression cassette does not comprise an additional transit peptide in case in Table I, application no. 7, in column 6 in the same line as SEQ ID NO. 22249 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 22611, or a homolog or fragment thereof, or a homolog thereof being depicted in Table I, application no. 7, column 8, in the same line as SEQ ID NO. 22611, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 22611 is depicted, plastidic is mentioned, or a mitochondrial transit peptide in case in Table I, application no. 7, in column 6 in the same line as SEQ ID NO. 22611 is depicted, mitochondric is mentioned; which is operable linked, too.

In another embodiment thereof the expression cassette does not comprise an additional transit peptide in case in Table I, application no. 7, in column 6 in the same line as SEQ ID NO. 22611 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 1815, or a homolog or fragment thereof, or a homolog thereof being depicted in Table I, application no. 7, column 8, in the same line as SEQ ID NO. 1815, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 1815 is depicted, plastidic is mentioned, or a mitochondrial transit peptide in case in Table I, application no. 7, in column 6 in the same line as SEQ ID NO. 1815 is depicted, mitochondric is mentioned; which is operable linked, too.

In another embodiment thereof the expression cassette does not comprise an additional transit peptide in case in Table I, application no. 7, in column 6 in the same line as SEQ ID NO. 1815 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 2573, or a homolog or fragment thereof, or a homolog thereof being depicted in Table I, application no. 7, column 8, in the same line as SEQ ID NO. 2573, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 2573 is depicted, plastidic is mentioned, or a mitochondrial transit peptide in case in Table I, application no. 7, in column 6 in the same line as SEQ ID NO. 2573 is depicted, mitochondric is mentioned; which is operable linked, too.

In another embodiment thereof the expression cassette does not comprise an additional transit peptide in case in Table I, application no. 7, in column 6 in the same line as SEQ ID NO. 2573 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 2935, or a homolog or fragment thereof, or a homolog thereof being depicted in Table I, application no. 7, column 8, in the same line as SEQ ID NO. 2935, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 2935 is depicted, plastidic is mentioned, or a mitochondrial transit peptide in case in Table I, application no. 7, in column 6 in the same line as SEQ ID NO. 2935 is depicted, mitochondric is mentioned; which is operable linked, too.

In another embodiment thereof the expression cassette does not comprise an additional transit peptide in case in Table I, application no. 7, in column 6 in the same line as SEQ ID NO. 2935 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 109717, or a homolog or fragment thereof, or a homolog thereof being depicted in Table I, application no. 7, column 8, in the same line as SEQ ID NO. 109717, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 109717 is depicted, plastidic is mentioned, or a mitochondrial transit peptide in case in Table I, application no. 7, in column 6 in the same line as SEQ ID NO. 109717 is depicted, mitochondric is mentioned; which is operable linked, too.

In another embodiment thereof the expression cassette does not comprise an additional transit peptide in case in Table I, application no. 7, in column 6 in the same line as SEQ ID NO. 109717 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 23482, or a homolog or fragment thereof, or a homolog thereof being depicted in Table I, application no. 7, column 8, in the same line as SEQ ID NO. 23482, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 23482 is depicted, plastidic is mentioned, or a mitochondrial transit peptide in case in Table I, application no. 7, in column 6 in the same line as SEQ ID NO. 23482 is depicted, mitochondric is mentioned; which is operable linked, too.

In another embodiment thereof the expression cassette does not comprise an additional transit peptide in case in Table I, application no. 7, in column 6 in the same line as SEQ ID NO. 23482 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 3279, or a homolog or fragment thereof, or a homolog thereof being depicted in Table I, application no. 7, column 8, in the same line as SEQ ID NO. 3279, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 3279 is depicted, plastidic is mentioned, or a mitochondrial transit peptide in case in Table I, application no. 7, in column 6 in the same line as SEQ ID NO. 3279 is depicted, mitochondric is mentioned; which is operable linked, too.

In another embodiment thereof the expression cassette does not comprise an additional transit peptide in case in Table I, application no. 7, in column 6 in the same line as SEQ ID NO. 3279 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 23844, or a homolog or fragment thereof, or a homolog thereof being depicted in Table I, application no. 7, column 8, in the same line as SEQ ID NO. 23844, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 23844 is depicted, plastidic is mentioned, or a mitochondrial transit peptide in case in Table I, application no. 7, in column 6 in the same line as SEQ ID NO. 23844 is depicted, mitochondric is mentioned; which is operable linked, too.

In another embodiment thereof the expression cassette does not comprise an additional transit peptide in case in Table I, application no. 7, in column 6 in the same line as SEQ ID NO. 23844 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68849, or a homolog or fragment thereof, or a homolog thereof being depicted in Table I, application no. 7, column 8, in the same line as SEQ ID NO. 68849, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 68849 is depicted, plastidic is mentioned, or a mitochondrial transit peptide in case in Table I, application no. 7, in column 6 in the same line as SEQ ID NO. 68849 is depicted, mitochondric is mentioned; which is operable linked, too.

In another embodiment thereof the expression cassette does not comprise an additional transit peptide in case in Table I, application no. 7, in column 6 in the same line as SEQ ID NO. 68849 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 24311, or a homolog or fragment thereof, or a homolog thereof being depicted in Table I, application no. 7, column 8, in the same line as SEQ ID NO. 24311, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 24311 is depicted, plastidic is mentioned, or a mitochondrial transit peptide in case in Table I, application no. 7, in column 6 in the same line as SEQ ID NO. 24311 is depicted, mitochondric is mentioned; which is operable linked, too.

In another embodiment thereof the expression cassette does not comprise an additional transit peptide in case in Table I, application no. 7, in column 6 in the same line as SEQ ID NO. 24311 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4348, or a homolog or fragment thereof, or a homolog thereof being depicted in Table I, application no. 7, column 8, in the same line as SEQ ID NO. 4348, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 4348 is depicted, plastidic is mentioned, or a mitochondrial transit peptide in case in Table I, application no. 7, in column 6 in the same line as SEQ ID NO. 4348 is depicted, mitochondric is mentioned; which is operable linked, too.

In another embodiment thereof the expression cassette does not comprise an additional transit peptide in case in Table I, application no. 7, in column 6 in the same line as SEQ ID NO. 4348 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 70006, or a homolog or fragment thereof, or a homolog thereof being depicted in Table I, application no. 7, column 8, in the same line as SEQ ID NO. 70006, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 70006 is depicted, plastidic is mentioned, or a mitochondrial transit peptide in case in Table I, application no. 7, in column 6 in the same line as SEQ ID NO. 70006 is depicted, mitochondric is mentioned; which is operable linked, too.

In another embodiment thereof the expression cassette does not comprise an additional transit peptide in case in Table I, application no. 7, in column 6 in the same line as SEQ ID NO. 70006 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 5493, or a homolog or fragment thereof, or a homolog thereof being depicted in Table I, application no. 7, column 8, in the same line as SEQ ID NO. 5493, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 5493 is depicted, plastidic is mentioned, or a mitochondrial transit peptide in case in Table I, application no. 7, in column 6 in the same line as SEQ ID NO. 5493 is depicted, mitochondric is mentioned; which is operable linked, too.

In another embodiment thereof the expression cassette does not comprise an additional transit peptide in case in Table I, application no. 7, in column 6 in the same line as SEQ ID NO. 5493 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 110651, or a homolog or fragment thereof, or a homolog thereof being depicted in Table I, application no. 7, column 8, in the same line as SEQ ID NO. 110651, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 110651 is depicted, plastidic is mentioned, or a mitochondrial transit peptide in case in Table I, application no. 7, in column 6 in the same line as SEQ ID NO. 110651 is depicted, mitochondric is mentioned; which is operable linked, too.

In another embodiment thereof the expression cassette does not comprise an additional transit peptide in case in Table I, application no. 7, in column 6 in the same line as SEQ ID NO. 110651 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 29286, or a homolog or fragment thereof, or a homolog thereof being depicted in Table I, application no. 7, column 8, in the same line as SEQ ID NO. 29286, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 29286 is depicted, plastidic is mentioned, or a mitochondrial transit peptide in case in Table I, application no. 7, in column 6 in the same line as SEQ ID NO. 29286 is depicted, mitochondric is mentioned; which is operable linked, too.

In another embodiment thereof the expression cassette does not comprise an additional transit peptide in case in Table I, application no. 7, in column 6 in the same line as SEQ ID NO. 29286 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 111155, or a homolog or fragment thereof, or a homolog thereof being depicted in Table I, application no. 7, column 8, in the same line as SEQ ID NO. 111155, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 111155 is depicted, plastidic is mentioned, or a mitochondrial transit peptide in case in Table I, application no. 7, in column 6 in the same line as SEQ ID NO. 111155 is depicted, mitochondric is mentioned; which is operable linked, too.

In another embodiment thereof the expression cassette does not comprise an additional transit peptide in case in Table I, application no. 7, in column 6 in the same line as SEQ ID NO. 111155 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 102941, or a homolog or fragment thereof, or a homolog thereof being depicted in Table I, application no. 7, column 8, in the same line as SEQ ID NO. 102941, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 102941 is depicted, plastidic is mentioned, or a mitochondrial transit peptide in case in Table I, application no. 7, in column 6 in the same line as SEQ ID NO. 102941 is depicted, mitochondric is mentioned; which is operable linked, too.

In another embodiment thereof the expression cassette does not comprise an additional transit peptide in case in Table I, application no. 7, in column 6 in the same line as SEQ ID NO. 102941 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6510, or a homolog or fragment thereof, or a homolog thereof being depicted in Table I, application no. 7, column 8, in the same line as SEQ ID NO. 6510, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 6510 is depicted, plastidic is mentioned, or a mitochondrial transit peptide in case in Table I, application no. 7, in column 6 in the same line as SEQ ID NO. 6510 is depicted, mitochondric is mentioned; which is operable linked, too.

In another embodiment thereof the expression cassette does not comprise an additional transit peptide in case in Table I, application no. 7, in column 6 in the same line as SEQ ID NO. 6510 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 74729, or a homolog or fragment thereof, or a homolog thereof being depicted in Table I, application no. 7, column 8, in the same line as SEQ ID NO. 74729, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 74729 is depicted, plastidic is mentioned, or a mitochondrial transit peptide in case in Table I, application no. 7, in column 6 in the same line as SEQ ID NO. 74729 is depicted, mitochondric is mentioned; which is operable linked, too.

In another embodiment thereof the expression cassette does not comprise an additional transit peptide in case in Table I, application no. 7, in column 6 in the same line as SEQ ID NO. 74729 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 34889, or a homolog or fragment thereof, or a homolog thereof being depicted in Table I, application no. 7, column 8, in the same line as SEQ ID NO. 34889, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 34889 is depicted, plastidic is mentioned, or a mitochondrial transit peptide in case in Table I, application no. 7, in column 6 in the same line as SEQ ID NO. 34889 is depicted, mitochondric is mentioned; which is operable linked, too.

In another embodiment thereof the expression cassette does not comprise an additional transit peptide in case in Table I, application no. 7, in column 6 in the same line as SEQ ID NO. 34889 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 35482, or a homolog or fragment thereof, or a homolog thereof being depicted in Table I, application no. 7, column 8, in the same line as SEQ ID NO. 35482, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 35482 is depicted, plastidic is mentioned, or a mitochondrial transit peptide in case in Table I, application no. 7, in column 6 in the same line as SEQ ID NO. 35482 is depicted, mitochondric is mentioned; which is operable linked, too.

In another embodiment thereof the expression cassette does not comprise an additional transit peptide in case in Table I, application no. 7, in column 6 in the same line as SEQ ID NO. 35482 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7081, or a homolog or fragment thereof, or a homolog thereof being depicted in Table I, application no. 7, column 8, in the same line as SEQ ID NO. 7081, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 7081 is depicted, plastidic is mentioned, or a mitochondrial transit peptide in case in Table I, application no. 7, in column 6 in the same line as SEQ ID NO. 7081 is depicted, mitochondric is mentioned; which is operable linked, too.

In another embodiment thereof the expression cassette does not comprise an additional transit peptide in case in Table I, application no. 7, in column 6 in the same line as SEQ ID NO. 7081 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 111340, or a homolog or fragment thereof, or a homolog thereof being depicted in Table I, application no. 7, column 8, in the same line as SEQ ID NO. 111340, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 111340 is depicted, plastidic is mentioned, or a mitochondrial transit peptide in case in Table I, application no. 7, in column 6 in the same line as SEQ ID NO. 111340 is depicted, mitochondric is mentioned; which is operable linked, too.

In another embodiment thereof the expression cassette does not comprise an additional transit peptide in case in Table I, application no. 7, in column 6 in the same line as SEQ ID NO. 111340 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7333, or a homolog or fragment thereof, or a homolog thereof being depicted in Table I, application no. 7, column 8, in the same line as SEQ ID NO. 7333, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 7333 is depicted, plastidic is mentioned, or a mitochondrial transit peptide in case in Table I, application no. 7, in column 6 in the same line as SEQ ID NO. 7333 is depicted, mitochondric is mentioned; which is operable linked, too.

In another embodiment thereof the expression cassette does not comprise an additional transit peptide in case in Table I, application no. 7, in column 6 in the same line as SEQ ID NO. 7333 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 75286, or a homolog or fragment thereof, or a homolog thereof being depicted in Table I, application no. 7, column 8, in the same line as SEQ ID NO. 75286, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 75286 is depicted, plastidic is mentioned, or a mitochondrial transit peptide in case in Table I, application no. 7, in column 6 in the same line as SEQ ID NO. 75286 is depicted, mitochondric is mentioned; which is operable linked, too.

In another embodiment thereof the expression cassette does not comprise an additional transit peptide in case in Table I, application no. 7, in column 6 in the same line as SEQ ID NO. 75286 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7917, or a homolog or fragment thereof, or a homolog thereof being depicted in Table I, application no. 7, column 8, in the same line as SEQ ID NO. 7917, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 7917 is depicted, plastidic is mentioned, or a mitochondrial transit peptide in case in Table I, application no. 7, in column 6 in the same line as SEQ ID NO. 7917 is depicted, mitochondric is mentioned; which is operable linked, too.

In another embodiment thereof the expression cassette does not comprise an additional transit peptide in case in Table I, application no. 7, in column 6 in the same line as SEQ ID NO. 7917 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 37394, or a homolog or fragment thereof, or a homolog thereof being depicted in Table I, application no. 7, column 8, in the same line as SEQ ID NO. 37394, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 37394 is depicted, plastidic is mentioned, or a mitochondrial transit peptide in case in Table I, application no. 7, in column 6 in the same line as SEQ ID NO. 37394 is depicted, mitochondric is mentioned; which is operable linked, too.

In another embodiment thereof the expression cassette does not comprise an additional transit peptide in case in Table I, application no. 7, in column 6 in the same line as SEQ ID NO. 37394 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 111910, or a homolog or fragment thereof, or a homolog thereof being depicted in Table I, application no. 7, column 8, in the same line as SEQ ID NO. 111910, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 111910 is depicted, plastidic is mentioned, or a mitochondrial transit peptide in case in Table I, application no. 7, in column 6 in the same line as SEQ ID NO. 111910 is depicted, mitochondric is mentioned; which is operable linked, too.

In another embodiment thereof the expression cassette does not comprise an additional transit peptide in case in Table I, application no. 7, in column 6 in the same line as SEQ ID NO. 111910 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 112072, or a homolog or fragment thereof, or a homolog thereof being depicted in Table I, application no. 7, column 8, in the same line as SEQ ID NO. 112072, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 112072 is depicted, plastidic is mentioned, or a mitochondrial transit peptide in case in Table I, application no. 7, in column 6 in the same line as SEQ ID NO. 112072 is depicted, mitochondric is mentioned; which is operable linked, too.

In another embodiment thereof the expression cassette does not comprise an additional transit peptide in case in Table I, application no. 7, in column 6 in the same line as SEQ ID NO. 112072 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 112111, or a homolog or fragment thereof, or a homolog thereof being depicted in Table I, application no. 7, column 8, in the same line as SEQ ID NO. 112111, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 112111 is depicted, plastidic is mentioned, or a mitochondrial transit peptide in case in Table I, application no. 7, in column 6 in the same line as SEQ ID NO. 112111 is depicted, mitochondric is mentioned; which is operable linked, too.

In another embodiment thereof the expression cassette does not comprise an additional transit peptide in case in Table I, application no. 7, in column 6 in the same line as SEQ ID NO. 112111 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 77774, or a homolog or fragment thereof, or a homolog thereof being depicted in Table I, application no. 7, column 8, in the same line as SEQ ID NO. 77774, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 77774 is depicted, plastidic is mentioned, or a mitochondrial transit peptide in case in Table I, application no. 7, in column 6 in the same line as SEQ ID NO. 77774 is depicted, mitochondric is mentioned; which is operable linked, too.

In another embodiment thereof the expression cassette does not comprise an additional transit peptide in case in Table I, application no. 7, in column 6 in the same line as SEQ ID NO. 77774 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7947, or a homolog or fragment thereof, or a homolog thereof being depicted in Table I, application no. 7, column 8, in the same line as SEQ ID NO. 7947, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 7947 is depicted, plastidic is mentioned, or a mitochondrial transit peptide in case in Table I, application no. 7, in column 6 in the same line as SEQ ID NO. 7947 is depicted, mitochondric is mentioned; which is operable linked, too.

In another embodiment thereof the expression cassette does not comprise an additional transit peptide in case in Table I, application no. 7, in column 6 in the same line as SEQ ID NO. 7947 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38300, or a homolog or fragment thereof, or a homolog thereof being depicted in Table I, application no. 7, column 8, in the same line as SEQ ID NO. 38300, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 38300 is depicted, plastidic is mentioned, or a mitochondrial transit peptide in case in Table I, application no. 7, in column 6 in the same line as SEQ ID NO. 38300 is depicted, mitochondric is mentioned; which is operable linked, too.

In another embodiment thereof the expression cassette does not comprise an additional transit peptide in case in Table I, application no. 7, in column 6 in the same line as SEQ ID NO. 38300 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7992, or a homolog or fragment thereof, or a homolog thereof being depicted in Table I, application no. 7, column 8, in the same line as SEQ ID NO. 7992, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 7992 is depicted, plastidic is mentioned, or a mitochondrial transit peptide in case in Table I, application no. 7, in column 6 in the same line as SEQ ID NO. 7992 is depicted, mitochondric is mentioned; which is operable linked, too.

In another embodiment thereof the expression cassette does not comprise an additional transit peptide in case in Table I, application no. 7, in column 6 in the same line as SEQ ID NO. 7992 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 79056, or a homolog or fragment thereof, or a homolog thereof being depicted in Table I, application no. 7, column 8, in the same line as SEQ ID NO. 79056, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 79056 is depicted, plastidic is mentioned, or a mitochondrial transit peptide in case in Table I, application no. 7, in column 6 in the same line as SEQ ID NO. 79056 is depicted, mitochondric is mentioned; which is operable linked, too.

In another embodiment thereof the expression cassette does not comprise an additional transit peptide in case in Table I, application no. 7, in column 6 in the same line as SEQ ID NO. 79056 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 112169, or a homolog or fragment thereof, or a homolog thereof being depicted in Table I, application no. 7, column 8, in the same line as SEQ ID NO. 112169, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 112169 is depicted, plastidic is mentioned, or a mitochondrial transit peptide in case in Table I, application no. 7, in column 6 in the same line as SEQ ID NO. 112169 is depicted, mitochondric is mentioned; which is operable linked, too.

In another embodiment thereof the expression cassette does not comprise an additional transit peptide in case in Table I, application no. 7, in column 6 in the same line as SEQ ID NO. 112169 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 79217, or a homolog or fragment thereof, or a homolog thereof being depicted in Table I, application no. 7, column 8, in the same line as SEQ ID NO. 79217, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 79217 is depicted, plastidic is mentioned, or a mitochondrial transit peptide in case in Table I, application no. 7, in column 6 in the same line as SEQ ID NO. 79217 is depicted, mitochondric is mentioned; which is operable linked, too.

In another embodiment thereof the expression cassette does not comprise an additional transit peptide in case in Table I, application no. 7, in column 6 in the same line as SEQ ID NO. 79217 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 112193, or a homolog or fragment thereof, or a homolog thereof being depicted in Table I, application no. 7, column 8, in the same line as SEQ ID NO. 112193, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 112193 is depicted, plastidic is mentioned, or a mitochondrial transit peptide in case in Table I, application no. 7, in column 6 in the same line as SEQ ID NO. 112193 is depicted, mitochondric is mentioned; which is operable linked, too.

In another embodiment thereof the expression cassette does not comprise an additional transit peptide in case in Table I, application no. 7, in column 6 in the same line as SEQ ID NO. 112193 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39219, or a homolog or fragment thereof, or a homolog thereof being depicted in Table I, application no. 7, column 8, in the same line as SEQ ID NO. 39219, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 39219 is depicted, plastidic is mentioned, or a mitochondrial transit peptide in case in Table I, application no. 7, in column 6 in the same line as SEQ ID NO. 39219 is depicted, mitochondric is mentioned; which is operable linked, too.

In another embodiment thereof the expression cassette does not comprise an additional transit peptide in case in Table I, application no. 7, in column 6 in the same line as SEQ ID NO. 39219 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 99898, or a homolog or fragment thereof, or a homolog thereof being depicted in Table I, application no. 7, column 8, in the same line as SEQ ID NO. 99898, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 99898 is depicted, plastidic is mentioned, or a mitochondrial transit peptide in case in Table I, application no. 7, in column 6 in the same line as SEQ ID NO. 99898 is depicted, mitochondric is mentioned; which is operable linked, too.

In another embodiment thereof the expression cassette does not comprise an additional transit peptide in case in Table I, application no. 7, in column 6 in the same line as SEQ ID NO. 99898 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40637, or a homolog or fragment thereof, or a homolog thereof being depicted in Table I, application no. 7, column 8, in the same line as SEQ ID NO. 40637, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 40637 is depicted, plastidic is mentioned, or a mitochondrial transit peptide in case in Table I, application no. 7, in column 6 in the same line as SEQ ID NO. 40637 is depicted, mitochondric is mentioned; which is operable linked, too.

In another embodiment thereof the expression cassette does not comprise an additional transit peptide in case in Table I, application no. 7, in column 6 in the same line as SEQ ID NO. 40637 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40665, or a homolog or fragment thereof, or a homolog thereof being depicted in Table I, application no. 7, column 8, in the same line as SEQ ID NO. 40665, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 40665 is depicted, plastidic is mentioned, or a mitochondrial transit peptide in case in Table I, application no. 7, in column 6 in the same line as SEQ ID NO. 40665 is depicted, mitochondric is mentioned; which is operable linked, too.

In another embodiment thereof the expression cassette does not comprise an additional transit peptide in case in Table I, application no. 7, in column 6 in the same line as SEQ ID NO. 40665 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 9244, or a homolog or fragment thereof, or a homolog thereof being depicted in Table I, application no. 7, column 8, in the same line as SEQ ID NO. 9244, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 9244 is depicted, plastidic is mentioned, or a mitochondrial transit peptide in case in Table I, application no. 7, in column 6 in the same line as SEQ ID NO. 9244 is depicted, mitochondric is mentioned; which is operable linked, too.

In another embodiment thereof the expression cassette does not comprise an additional transit peptide in case in Table I, application no. 7, in column 6 in the same line as SEQ ID NO. 9244 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 80756, or a homolog or fragment thereof, or a homolog thereof being depicted in Table I, application no. 7, column 8, in the same line as SEQ ID NO. 80756, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 80756 is depicted, plastidic is mentioned, or a mitochondrial transit peptide in case in Table I, application no. 7, in column 6 in the same line as SEQ ID NO. 80756 is depicted, mitochondric is mentioned; which is operable linked, too.

In another embodiment thereof the expression cassette does not comprise an additional transit peptide in case in Table I, application no. 7, in column 6 in the same line as SEQ ID NO. 80756 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40795, or a homolog or fragment thereof, or a homolog thereof being depicted in Table I, application no. 7, column 8, in the same line as SEQ ID NO. 40795, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 40795 is depicted, plastidic is mentioned, or a mitochondrial transit peptide in case in Table I, application no. 7, in column 6 in the same line as SEQ ID NO. 40795 is depicted, mitochondric is mentioned; which is operable linked, too.

In another embodiment thereof the expression cassette does not comprise an additional transit peptide in case in Table I, application no. 7, in column 6 in the same line as SEQ ID NO. 40795 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 81576, or a homolog or fragment thereof, or a homolog thereof being depicted in Table I, application no. 7, column 8, in the same line as SEQ ID NO. 81576, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 81576 is depicted, plastidic is mentioned, or a mitochondrial transit peptide in case in Table I, application no. 7, in column 6 in the same line as SEQ ID NO. 81576 is depicted, mitochondric is mentioned; which is operable linked, too.

In another embodiment thereof the expression cassette does not comprise an additional transit peptide in case in Table I, application no. 7, in column 6 in the same line as SEQ ID NO. 81576 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 112216, or a homolog or fragment thereof, or a homolog thereof being depicted in Table I, application no. 7, column 8, in the same line as SEQ ID NO. 112216, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 112216 is depicted, plastidic is mentioned, or a mitochondrial transit peptide in case in Table I, application no. 7, in column 6 in the same line as SEQ ID NO. 112216 is depicted, mitochondric is mentioned; which is operable linked, too.

In another embodiment thereof the expression cassette does not comprise an additional transit peptide in case in Table I, application no. 7, in column 6 in the same line as SEQ ID NO. 112216 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 112380, or a homolog or fragment thereof, or a homolog thereof being depicted in Table I, application no. 7, column 8, in the same line as SEQ ID NO. 112380, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 112380 is depicted, plastidic is mentioned, or a mitochondrial transit peptide in case in Table I, application no. 7, in column 6 in the same line as SEQ ID NO. 112380 is depicted, mitochondric is mentioned; which is operable linked, too.

In another embodiment thereof the expression cassette does not comprise an additional transit peptide in case in Table I, application no. 7, in column 6 in the same line as SEQ ID NO. 112380 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 112606, or a homolog or fragment thereof, or a homolog thereof being depicted in Table I, application no. 7, column 8, in the same line as SEQ ID NO. 112606, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 112606 is depicted, plastidic is mentioned, or a mitochondrial transit peptide in case in Table I, application no. 7, in column 6 in the same line as SEQ ID NO. 112606 is depicted, mitochondric is mentioned; which is operable linked, too.

In another embodiment thereof the expression cassette does not comprise an additional transit peptide in case in Table I, application no. 7, in column 6 in the same line as SEQ ID NO. 112606 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 112634, or a homolog or fragment thereof, or a homolog thereof being depicted in Table I, application no. 7, column 8, in the same line as SEQ ID NO. 112634, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 112634 is depicted, plastidic is mentioned, or a mitochondrial transit peptide in case in Table I, application no. 7, in column 6 in the same line as SEQ ID NO. 112634 is depicted, mitochondric is mentioned; which is operable linked, too.

In another embodiment thereof the expression cassette does not comprise an additional transit peptide in case in Table I, application no. 7, in column 6 in the same line as SEQ ID NO. 112634 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 44223, or a homolog or fragment thereof, or a homolog thereof being depicted in Table I, application no. 7, column 8, in the same line as SEQ ID NO. 44223, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 44223 is depicted, plastidic is mentioned, or a mitochondrial transit peptide in case in Table I, application no. 7, in column 6 in the same line as SEQ ID NO. 44223 is depicted, mitochondric is mentioned; which is operable linked, too.

In another embodiment thereof the expression cassette does not comprise an additional transit peptide in case in Table I, application no. 7, in column 6 in the same line as SEQ ID NO. 44223 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 44372, or a homolog or fragment thereof, or a homolog thereof being depicted in Table I, application no. 7, column 8, in the same line as SEQ ID NO. 44372, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 44372 is depicted, plastidic is mentioned, or a mitochondrial transit peptide in case in Table I, application no. 7, in column 6 in the same line as SEQ ID NO. 44372 is depicted, mitochondric is mentioned; which is operable linked, too.

In another embodiment thereof the expression cassette does not comprise an additional transit peptide in case in Table I, application no. 7, in column 6 in the same line as SEQ ID NO. 44372 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 10740, or a homolog or fragment thereof, or a homolog thereof being depicted in Table I, application no. 7, column 8, in the same line as SEQ ID NO. 10740, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 10740 is depicted, plastidic is mentioned, or a mitochondrial transit peptide in case in Table I, application no. 7, in column 6 in the same line as SEQ ID NO. 10740 is depicted, mitochondric is mentioned; which is operable linked, too.

In another embodiment thereof the expression cassette does not comprise an additional transit peptide in case in Table I, application no. 7, in column 6 in the same line as SEQ ID NO. 10740 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 106532, or a homolog or fragment thereof, or a homolog thereof being depicted in Table I, application no. 7, column 8, in the same line as SEQ ID NO. 106532, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 106532 is depicted, plastidic is mentioned, or a mitochondrial transit peptide in case in Table I, application no. 7, in column 6 in the same line as SEQ ID NO. 106532 is depicted, mitochondric is mentioned; which is operable linked, too.

In another embodiment thereof the expression cassette does not comprise an additional transit peptide in case in Table I, application no. 7, in column 6 in the same line as SEQ ID NO. 106532 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 94542, or a homolog or fragment thereof, or a homolog thereof being depicted in Table I, application no. 7, column 8, in the same line as SEQ ID NO. 94542, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 94542 is depicted, plastidic is mentioned, or a mitochondrial transit peptide in case in Table I, application no. 7, in column 6 in the same line as SEQ ID NO. 94542 is depicted, mitochondric is mentioned; which is operable linked, too.

In another embodiment thereof the expression cassette does not comprise an additional transit peptide in case in Table I, application no. 7, in column 6 in the same line as SEQ ID NO. 94542 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 45321, or a homolog or fragment thereof, or a homolog thereof being depicted in Table I, application no. 7, column 8, in the same line as SEQ ID NO. 45321, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 45321 is depicted, plastidic is mentioned, or a mitochondrial transit peptide in case in Table I, application no. 7, in column 6 in the same line as SEQ ID NO. 45321 is depicted, mitochondric is mentioned; which is operable linked, too.

In another embodiment thereof the expression cassette does not comprise an additional transit peptide in case in Table I, application no. 7, in column 6 in the same line as SEQ ID NO. 45321 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 45394, or a homolog or fragment thereof, or a homolog thereof being depicted in Table I, application no. 7, column 8, in the same line as SEQ ID NO. 45394, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 45394 is depicted, plastidic is mentioned, or a mitochondrial transit peptide in case in Table I, application no. 7, in column 6 in the same line as SEQ ID NO. 45394 is depicted, mitochondric is mentioned; which is operable linked, too.

In another embodiment thereof the expression cassette does not comprise an additional transit peptide in case in Table I, application no. 7, in column 6 in the same line as SEQ ID NO. 45394 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 45757, or a homolog or fragment thereof, or a homolog thereof being depicted in Table I, application no. 7, column 8, in the same line as SEQ ID NO. 45757, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 45757 is depicted, plastidic is mentioned, or a mitochondrial transit peptide in case in Table I, application no. 7, in column 6 in the same line as SEQ ID NO. 45757 is depicted, mitochondric is mentioned; which is operable linked, too.

In another embodiment thereof the expression cassette does not comprise an additional transit peptide in case in Table I, application no. 7, in column 6 in the same line as SEQ ID NO. 45757 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 10811, or a homolog or fragment thereof, or a homolog thereof being depicted in Table I, application no. 7, column 8, in the same line as SEQ ID NO. 10811, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 10811 is depicted, plastidic is mentioned, or a mitochondrial transit peptide in case in Table I, application no. 7, in column 6 in the same line as SEQ ID NO. 10811 is depicted, mitochondric is mentioned; which is operable linked, too.

In another embodiment thereof the expression cassette does not comprise an additional transit peptide in case in Table I, application no. 7, in column 6 in the same line as SEQ ID NO. 10811 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 84198, or a homolog or fragment thereof, or a homolog thereof being depicted in Table I, application no. 7, column 8, in the same line as SEQ ID NO. 84198, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 84198 is depicted, plastidic is mentioned, or a mitochondrial transit peptide in case in Table I, application no. 7, in column 6 in the same line as SEQ ID NO. 84198 is depicted, mitochondric is mentioned; which is operable linked, too.

In another embodiment thereof the expression cassette does not comprise an additional transit peptide in case in Table I, application no. 7, in column 6 in the same line as SEQ ID NO. 84198 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 49828, or a homolog or fragment thereof, or a homolog thereof being depicted in Table I, application no. 7, column 8, in the same line as SEQ ID NO. 49828, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 49828 is depicted, plastidic is mentioned, or a mitochondrial transit peptide in case in Table I, application no. 7, in column 6 in the same line as SEQ ID NO. 49828 is depicted, mitochondric is mentioned; which is operable linked, too.

In another embodiment thereof the expression cassette does not comprise an additional transit peptide in case in Table I, application no. 7, in column 6 in the same line as SEQ ID NO. 49828 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 112661, or a homolog or fragment thereof, or a homolog thereof being depicted in Table I, application no. 7, column 8, in the same line as SEQ ID NO. 112661, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 112661 is depicted, plastidic is mentioned, or a mitochondrial transit peptide in case in Table I, application no. 7, in column 6 in the same line as SEQ ID NO. 112661 is depicted, mitochondric is mentioned; which is operable linked, too.

In another embodiment thereof the expression cassette does not comprise an additional transit peptide in case in Table I, application no. 7, in column 6 in the same line as SEQ ID NO. 112661 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 53189, or a homolog or fragment thereof, or a homolog thereof being depicted in Table I, application no. 7, column 8, in the same line as SEQ ID NO. 53189, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 53189 is depicted, plastidic is mentioned, or a mitochondrial transit peptide in case in Table I, application no. 7, in column 6 in the same line as SEQ ID NO. 53189 is depicted, mitochondric is mentioned; which is operable linked, too.

In another embodiment thereof the expression cassette does not comprise an additional transit peptide in case in Table I, application no. 7, in column 6 in the same line as SEQ ID NO. 53189 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 55379, or a homolog or fragment thereof, or a homolog thereof being depicted in Table I, application no. 7, column 8, in the same line as SEQ ID NO. 55379, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 55379 is depicted, plastidic is mentioned, or a mitochondrial transit peptide in case in Table I, application no. 7, in column 6 in the same line as SEQ ID NO. 55379 is depicted, mitochondric is mentioned; which is operable linked, too.

In another embodiment thereof the expression cassette does not comprise an additional transit peptide in case in Table I, application no. 7, in column 6 in the same line as SEQ ID NO. 55379 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 85808, or a homolog or fragment thereof, or a homolog thereof being depicted in Table I, application no. 7, column 8, in the same line as SEQ ID NO. 85808, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 85808 is depicted, plastidic is mentioned, or a mitochondrial transit peptide in case in Table I, application no. 7, in column 6 in the same line as SEQ ID NO. 85808 is depicted, mitochondric is mentioned; which is operable linked, too.

In another embodiment thereof the expression cassette does not comprise an additional transit peptide in case in Table I, application no. 7, in column 6 in the same line as SEQ ID NO. 85808 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 86540, or a homolog or fragment thereof, or a homolog thereof being depicted in Table I, application no. 7, column 8, in the same line as SEQ ID NO. 86540, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 86540 is depicted, plastidic is mentioned, or a mitochondrial transit peptide in case in Table I, application no. 7, in column 6 in the same line as SEQ ID NO. 86540 is depicted, mitochondric is mentioned; which is operable linked, too.

In another embodiment thereof the expression cassette does not comprise an additional transit peptide in case in Table I, application no. 7, in column 6 in the same line as SEQ ID NO. 86540 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 87397, or a homolog or fragment thereof, or a homolog thereof being depicted in Table I, application no. 7, column 8, in the same line as SEQ ID NO. 87397, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 87397 is depicted, plastidic is mentioned, or a mitochondrial transit peptide in case in Table I, application no. 7, in column 6 in the same line as SEQ ID NO. 87397 is depicted, mitochondric is mentioned; which is operable linked, too.

In another embodiment thereof the expression cassette does not comprise an additional transit peptide in case in Table I, application no. 7, in column 6 in the same line as SEQ ID NO. 87397 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 57235, or a homolog or fragment thereof, or a homolog thereof being depicted in Table I, application no. 7, column 8, in the same line as SEQ ID NO. 57235, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 57235 is depicted, plastidic is mentioned, or a mitochondrial transit peptide in case in Table I, application no. 7, in column 6 in the same line as SEQ ID NO. 57235 is depicted, mitochondric is mentioned; which is operable linked, too.

In another embodiment thereof the expression cassette does not comprise an additional transit peptide in case in Table I, application no. 7, in column 6 in the same line as SEQ ID NO. 57235 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 58590, or a homolog or fragment thereof, or a homolog thereof being depicted in Table I, application no. 7, column 8, in the same line as SEQ ID NO. 58590, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 58590 is depicted, plastidic is mentioned, or a mitochondrial transit peptide in case in Table I, application no. 7, in column 6 in the same line as SEQ ID NO. 58590 is depicted, mitochondric is mentioned; which is operable linked, too.

In another embodiment thereof the expression cassette does not comprise an additional transit peptide in case in Table I, application no. 7, in column 6 in the same line as SEQ ID NO. 58590 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 58731, or a homolog or fragment thereof, or a homolog thereof being depicted in Table I, application no. 7, column 8, in the same line as SEQ ID NO. 58731, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 58731 is depicted, plastidic is mentioned, or a mitochondrial transit peptide in case in Table I, application no. 7, in column 6 in the same line as SEQ ID NO. 58731 is depicted, mitochondric is mentioned; which is operable linked, too.

In another embodiment thereof the expression cassette does not comprise an additional transit peptide in case in Table I, application no. 7, in column 6 in the same line as SEQ ID NO. 58731 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 58823, or a homolog or fragment thereof, or a homolog thereof being depicted in Table I, application no. 7, column 8, in the same line as SEQ ID NO. 58823, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 58823 is depicted, plastidic is mentioned, or a mitochondrial transit peptide in case in Table I, application no. 7, in column 6 in the same line as SEQ ID NO. 58823 is depicted, mitochondric is mentioned; which is operable linked, too.

In another embodiment thereof the expression cassette does not comprise an additional transit peptide in case in Table I, application no. 7, in column 6 in the same line as SEQ ID NO. 58823 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12140, or a homolog or fragment thereof, or a homolog thereof being depicted in Table I, application no. 7, column 8, in the same line as SEQ ID NO. 12140, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 12140 is depicted, plastidic is mentioned, or a mitochondrial transit peptide in case in Table I, application no. 7, in column 6 in the same line as SEQ ID NO. 12140 is depicted, mitochondric is mentioned; which is operable linked, too.

In another embodiment thereof the expression cassette does not comprise an additional transit peptide in case in Table I, application no. 7, in column 6 in the same line as SEQ ID NO. 12140 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 113038, or a homolog or fragment thereof, or a homolog thereof being depicted in Table I, application no. 7, column 8, in the same line as SEQ ID NO. 113038, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 113038 is depicted, plastidic is mentioned, or a mitochondrial transit peptide in case in Table I, application no. 7, in column 6 in the same line as SEQ ID NO. 113038 is depicted, mitochondric is mentioned; which is operable linked, too.

In another embodiment thereof the expression cassette does not comprise an additional transit peptide in case in Table I, application no. 7, in column 6 in the same line as SEQ ID NO. 113038 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12698, or a homolog or fragment thereof, or a homolog thereof being depicted in Table I, application no. 7, column 8, in the same line as SEQ ID NO. 12698, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 12698 is depicted, plastidic is mentioned, or a mitochondrial transit peptide in case in Table I, application no. 7, in column 6 in the same line as SEQ ID NO. 12698 is depicted, mitochondric is mentioned; which is operable linked, too.

In another embodiment thereof the expression cassette does not comprise an additional transit peptide in case in Table I, application no. 7, in column 6 in the same line as SEQ ID NO. 12698 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 61723, or a homolog or fragment thereof, or a homolog thereof being depicted in Table I, application no. 7, column 8, in the same line as SEQ ID NO. 61723, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 61723 is depicted, plastidic is mentioned, or a mitochondrial transit peptide in case in Table I, application no. 7, in column 6 in the same line as SEQ ID NO. 61723 is depicted, mitochondric is mentioned; which is operable linked, too.

In another embodiment thereof the expression cassette does not comprise an additional transit peptide in case in Table I, application no. 7, in column 6 in the same line as SEQ ID NO. 61723 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 62079, or a homolog or fragment thereof, or a homolog thereof being depicted in Table I, application no. 7, column 8, in the same line as SEQ ID NO. 62079, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 62079 is depicted, plastidic is mentioned, or a mitochondrial transit peptide in case in Table I, application no. 7, in column 6 in the same line as SEQ ID NO. 62079 is depicted, mitochondric is mentioned; which is operable linked, too.

In another embodiment thereof the expression cassette does not comprise an additional transit peptide in case in Table I, application no. 7, in column 6 in the same line as SEQ ID NO. 62079 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12974, or a homolog or fragment thereof, or a homolog thereof being depicted in Table I, application no. 7, column 8, in the same line as SEQ ID NO. 12974, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 12974 is depicted, plastidic is mentioned, or a mitochondrial transit peptide in case in Table I, application no. 7, in column 6 in the same line as SEQ ID NO. 12974 is depicted, mitochondric is mentioned; which is operable linked, too.

In another embodiment thereof the expression cassette does not comprise an additional transit peptide in case in Table I, application no. 7, in column 6 in the same line as SEQ ID NO. 12974 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 88879, or a homolog or fragment thereof, or a homolog thereof being depicted in Table I, application no. 7, column 8, in the same line as SEQ ID NO. 88879, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 88879 is depicted, plastidic is mentioned, or a mitochondrial transit peptide in case in Table I, application no. 7, in column 6 in the same line as SEQ ID NO. 88879 is depicted, mitochondric is mentioned; which is operable linked, too.

In another embodiment thereof the expression cassette does not comprise an additional transit peptide in case in Table I, application no. 7, in column 6 in the same line as SEQ ID NO. 88879 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 113220, or a homolog or fragment thereof, or a homolog thereof being depicted in Table I, application no. 7, column 8, in the same line as SEQ ID NO. 113220, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 113220 is depicted, plastidic is mentioned, or a mitochondrial transit peptide in case in Table I, application no. 7, in column 6 in the same line as SEQ ID NO. 113220 is depicted, mitochondric is mentioned; which is operable linked, too.

In another embodiment thereof the expression cassette does not comprise an additional transit peptide in case in Table I, application no. 7, in column 6 in the same line as SEQ ID NO. 113220 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 113224, or a homolog or fragment thereof, or a homolog thereof being depicted in Table I, application no. 7, column 8, in the same line as SEQ ID NO. 113224, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 113224 is depicted, plastidic is mentioned, or a mitochondrial transit peptide in case in Table I, application no. 7, in column 6 in the same line as SEQ ID NO. 113224 is depicted, mitochondric is mentioned; which is operable linked, too.

In another embodiment thereof the expression cassette does not comprise an additional transit peptide in case in Table I, application no. 7, in column 6 in the same line as SEQ ID NO. 113224 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 113313, or a homolog or fragment thereof, or a homolog thereof being depicted in Table I, application no. 7, column 8, in the same line as SEQ ID NO. 113313, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 113313 is depicted, plastidic is mentioned, or a mitochondrial transit peptide in case in Table I, application no. 7, in column 6 in the same line as SEQ ID NO. 113313 is depicted, mitochondric is mentioned; which is operable linked, too.

In another embodiment thereof the expression cassette does not comprise an additional transit peptide in case in Table I, application no. 7, in column 6 in the same line as SEQ ID NO. 113313 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 89973, or a homolog or fragment thereof, or a homolog thereof being depicted in Table I, application no. 7, column 8, in the same line as SEQ ID NO. 89973, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 89973 is depicted, plastidic is mentioned, or a mitochondrial transit peptide in case in Table I, application no. 7, in column 6 in the same line as SEQ ID NO. 89973 is depicted, mitochondric is mentioned; which is operable linked, too.

In another embodiment thereof the expression cassette does not comprise an additional transit peptide in case in Table I, application no. 7, in column 6 in the same line as SEQ ID NO. 89973 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 113330, or a homolog or fragment thereof, or a homolog thereof being depicted in Table I, application no. 7, column 8, in the same line as SEQ ID NO. 113330, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 113330 is depicted, plastidic is mentioned, or a mitochondrial transit peptide in case in Table I, application no. 7, in column 6 in the same line as SEQ ID NO. 113330 is depicted, mitochondric is mentioned; which is operable linked, too.

In another embodiment thereof the expression cassette does not comprise an additional transit peptide in case in Table I, application no. 7, in column 6 in the same line as SEQ ID NO. 113330 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 90103, or a homolog or fragment thereof, or a homolog thereof being depicted in Table I, application no. 7, column 8, in the same line as SEQ ID NO. 90103, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 90103 is depicted, plastidic is mentioned, or a mitochondrial transit peptide in case in Table I, application no. 7, in column 6 in the same line as SEQ ID NO. 90103 is depicted, mitochondric is mentioned; which is operable linked, too.

In another embodiment thereof the expression cassette does not comprise an additional transit peptide in case in Table I, application no. 7, in column 6 in the same line as SEQ ID NO. 90103 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 63803, or a homolog or fragment thereof, or a homolog thereof being depicted in Table I, application no. 7, column 8, in the same line as SEQ ID NO. 63803, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 63803 is depicted, plastidic is mentioned, or a mitochondrial transit peptide in case in Table I, application no. 7, in column 6 in the same line as SEQ ID NO. 63803 is depicted, mitochondric is mentioned; which is operable linked, too.

In another embodiment thereof the expression cassette does not comprise an additional transit peptide in case in Table I, application no. 7, in column 6 in the same line as SEQ ID NO. 63803 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 113598, or a homolog or fragment thereof, or a homolog thereof being depicted in Table I, application no. 7, column 8, in the same line as SEQ ID NO. 113598, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 113598 is depicted, plastidic is mentioned, or a mitochondrial transit peptide in case in Table I, application no. 7, in column 6 in the same line as SEQ ID NO. 113598 is depicted, mitochondric is mentioned; which is operable linked, too.

In another embodiment thereof the expression cassette does not comprise an additional transit peptide in case in Table I, application no. 7, in column 6 in the same line as SEQ ID NO. 113598 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 113636, or a homolog or fragment thereof, or a homolog thereof being depicted in Table I, application no. 7, column 8, in the same line as SEQ ID NO. 113636, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 113636 is depicted, plastidic is mentioned, or a mitochondrial transit peptide in case in Table I, application no. 7, in column 6 in the same line as SEQ ID NO. 113636 is depicted, mitochondric is mentioned; which is operable linked, too.

In another embodiment thereof the expression cassette does not comprise an additional transit peptide in case in Table I, application no. 7, in column 6 in the same line as SEQ ID NO. 113636 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 66274, or a homolog or fragment thereof, or a homolog thereof being depicted in Table I, application no. 7, column 8, in the same line as SEQ ID NO. 66274, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 66274 is depicted, plastidic is mentioned, or a mitochondrial transit peptide in case in Table I, application no. 7, in column 6 in the same line as SEQ ID NO. 66274 is depicted, mitochondric is mentioned; which is operable linked, too.

In another embodiment thereof the expression cassette does not comprise an additional transit peptide in case in Table I, application no. 7, in column 6 in the same line as SEQ ID NO. 66274 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 66715, or a homolog or fragment thereof, or a homolog thereof being depicted in Table I, application no. 7, column 8, in the same line as SEQ ID NO. 66715, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 66715 is depicted, plastidic is mentioned, or a mitochondrial transit peptide in case in Table I, application no. 7, in column 6 in the same line as SEQ ID NO. 66715 is depicted, mitochondric is mentioned; which is operable linked, too.

In another embodiment thereof the expression cassette does not comprise an additional transit peptide in case in Table I, application no. 7, in column 6 in the same line as SEQ ID NO. 66715 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 67190, or a homolog or fragment thereof, or a homolog thereof being depicted in Table I, application no. 7, column 8, in the same line as SEQ ID NO. 67190, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 67190 is depicted, plastidic is mentioned, or a mitochondrial transit peptide in case in Table I, application no. 7, in column 6 in the same line as SEQ ID NO. 67190 is depicted, mitochondric is mentioned; which is operable linked, too.

In another embodiment thereof the expression cassette does not comprise an additional transit peptide in case in Table I, application no. 7, in column 6 in the same line as SEQ ID NO. 67190 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 113647, or a homolog or fragment thereof, or a homolog thereof being depicted in Table I, application no. 7, column 8, in the same line as SEQ ID NO. 113647, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 7, in column 6 in the same line as SEQ ID NO. 113647 is depicted, plastidic is mentioned, or a mitochondrial transit peptide in case in Table I, application no. 7, in column 6 in the same line as SEQ ID NO. 113647 is depicted, mitochondric is mentioned; which is operable linked, too.

In another embodiment thereof the expression cassette does not comprise an additional transit peptide in case in Table I, application no. 7, in column 6 in the same line as SEQ ID NO. 113647 is depicted, non-targeted is mentioned.

Accordingly, in one embodiment, the process according to the invention comprises the following steps:
(a) introducing of a nucleic acid construct comprising the nucleic acid molecule of the invention (and if desired, in combination with the nucleic acid encoding a respective transit peptide) or used in the process of the invention or encoding the polypeptide of the present invention or used in the process of the invention; or
(b) introducing of a nucleic acid molecule, including regulatory sequences and/or factors (and if desired, in combination with the nucleic acid encoding a respective transit peptide), which expression increases the expression of the nucleic acid molecule of the invention or used in the process of the invention or encoding the polypeptide of the present invention or used in the process of the invention;
in a cell, or a non-human organism or a part thereof, preferably in a plant, plant cell or a microorganism, preferably in the organelles such as the plastids or mitochondria thereof, or preferably non-targeted, or
(c) introducing an expression cassette as mentioned above,
and
(d) expressing of the gene product encoded by the nucleic acid construct or the nucleic acid molecule (and if desired, in combination with the nucleic acid encoding a respective transit peptide) or expression cassette mentioned under (a), (b) or (c) in the cell or the non-human organism or part thereof.

For expression in a host organism, for example a plant, the expression cassette is advantageously inserted into a vector such as by way of example a plasmid, a phage or other DNA which allows optimal expression of the genes in the host organism. Examples of suitable plasmids are: in E. coli pLG338, pACYC184, pBR series such as e.g. pBR322, pUC series such as pUC18 or pUC19, M113mp series, pKC30, pRep4, pHS1, pHS2, pPLc236, pMBL24, pLG200, pUR290, pIN-III113-B1, λgt11 or pBdCI; in Streptomyces pIJ101, pIJ364, pIJ702 or pIJ361; in Bacillus pUB110, pC194 or pBD214; in Corynebacterium pSA77 or pAJ667; in fungi pALS1, pIL2 or pBB116; other advantageous fungal vectors are described by Romanos M. A. et al., Yeast 8, 423 (1992) and by van den Hondel C. A. M. J. J. et al. [(1991) “Heterologous gene expression in filamentous fungi”] as well as in “More Gene Manipulations” in “Fungi” in Bennet J. W. & Lasure L. L., eds., pp. 396-428, Academic Press, San Diego, and in “Gene transfer systems and vector development for filamentous fungi” [van den Hondel C. A. M. J. J. & Punt P. J. (1991) in: Applied Molecular Genetics of Fungi, Peberdy J. F. et al., eds., pp. 1-28, Cambridge University Press: Cambridge]. Examples of advantageous yeast vectors are 2 μM, pAG-1, YEp6, YEp13 or pEMBLYe23. Examples of algal or plant plasmids are pLGV23, pGHlac+, pBIN19, pAK2004, pVKH or pDH51 (see Schmidt R., Willmitzer L., Plant Cell Rep. 7, 583 (1988)). The vectors identified above or derivatives of the vectors identified above are a small selection of the possible plasmids. Further plasmids are well known to those skilled in the art and may be found, for example, in “Cloning Vectors” (Eds. P. H. Pouwels et al., Elsevier, Amsterdam-New York-Oxford, 1985, ISBN 0 444 904018). Suitable plant vectors are described inter alia in “Methods in Plant Molecular Biology and Biotechnology” (CRC Press, Ch. 6/7, pp. 71-119). Advantageous vectors are known as shuttle vectors or binary vectors which replicate in E. coli and Agrobacterium. Examples for binary vectors are pBIN19, pBI101, pBinAR, pGPTV, pCAMBIA, pBIB-HYG, pBecks, pGreen or pPZP (Hajukiewicz P. et al., Plant Mol. Biol. 25, 989 (1994), and Hellens et al, Trends in Plant Science 5, 446 (2000)).

In a further embodiment of the vector the expression cassette according to the invention may also advantageously be introduced into the organisms in the form of a linear DNA and be integrated into the genome of the host organism by way of heterologous or homologous recombination. This linear DNA may be composed of a linearized plasmid or only of the expression cassette as vector or the respective nucleic acid sequences (if desired, in combination with the nucleic acid encoding a respective transit peptide) according to the invention.

In a further advantageous embodiment the nucleic acid sequence (if desired, in combination with the nucleic acid encoding a respective transit peptide) according to the invention can also be introduced into an organism on its own.

If in addition to the nucleic acid sequence according to the invention (and if desired, in combination with the nucleic acid encoding a respective transit peptide) further genes are to be introduced into the organism, all genes altogether with a marker in a single vector or each single gene with a marker in a vector can be introduced into the organism, whereby the different vectors can be introduced simultaneously or successively.

The vector advantageously contains at least one copy of the nucleic acid sequences according to the invention (and if desired, in combination with the nucleic acid encoding a respective transit peptide) or of the expression cassette (=gene construct) according to the invention.

The invention further provides a vector, comprising a nucleic acid molecule comprising 1) a nucleic acid molecule selected from the group consisting of:
(a) a nucleic acid molecule encoding a polypeptide shown in Table II, column 5 or 8, application no. 7, preferably shown in Table II A, application no. 7, in column 5, or in Table II A, application no. 7, column 8, or in Table II B, application no. 7, column 8, or a homolog or a fragment thereof;
(b) a nucleic acid molecule shown in Table I, application no. 7, column 5 or 8, preferably shown in Table I A, application no. 7, in column 5, or in Table I A, application no. 7, column 8, or in Table I B, application no. 7, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 7, preferably shown in Table II A, application no. 7, in column 5, or in Table II A, application no. 7, column 8, or in Table II B, application no. 7, column 8;
(d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 7, preferably shown in Table I A, application no. 7, in column 5, or in Table I A, application no. 7, column 8, or in Table I B, application no. 7, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 7;
(i) a nucleic acid molecule encoding a polypeptide having the activity represented by a protein as depicted in column 5 of Table II, application no. 7;
(j) a nucleic acid molecule comprising a polynucleotide which is obtained by amplifying a cDNA library or a genomic library using the primers in Table III, application no. 7, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 7.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 7, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 7.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 7, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 7, in the same line as of the respective nucleic acid molecule according to 1) “plastidic” is depicted, or encoding a mitochondrial transit peptide, especially in case in column 6 of Table I, application no. 7, in the same line as of the respective nucleic acid molecule according to 1) “mitochondric” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 7, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 7, in the same line as of the respective nucleic acid molecule according to 1) “plastidic” is depicted, or encoding a mitochondrial transit peptide, especially in case in column 6 of Table I, application no. 7, in the same line as of the respective nucleic acid molecule according to 1) “mitochondric” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operable linked; a nucleic acid molecule encoding a transit peptide, especially in case in column 6 of Table I, application no. 7, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 7, in the same line as of the respective nucleic acid molecule according to 1) “plastidic” is depicted, or encoding a mitochondrial transit peptide, especially in case in column 6 of Table I, application no. 7, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 7 (in the same line).

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 7, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 7.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 7, in the same line as of the respective nucleic acid molecule according to 1) “plastidic” is depicted, or encoding a mitochondrial transit peptide, especially in case in column 6 of Table I, application no. 7, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 7, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 7, in the same line as of the respective nucleic acid molecule according to 1) “plastidic” is depicted, or encoding a mitochondrial transit peptide, especially in case in column 6 of Table I, application no. 7, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 7 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 7, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 7 (in the same line) and confers a generation or an increase of the respective fine chemical in a non-human organism or a part thereof as compared to a corresponding wild-type non-human organism or part thereof.

In a preferred embodiment of said vector said nucleic acid molecule according to 1 (a), (b), (c), (d), (e), (f), (g), (h), (i), (j) or (k), said promoter and optionally said nucleic acid molecule encoding a transit peptide are encompassed in an expression cassette. Accordingly in an embodiment the invention provides a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

The invention further provides a vector comprising a nucleic acid molecule comprising 1) a nucleic acid molecule selected from the group consisting of:
(a) a nucleic acid molecule encoding a polypeptide shown in Table II, column 5 or 8, application no. 7, preferably shown in Table II A, application no. 7, in column 5, or in Table II A, application no. 7, column 8, or in Table II B, application no. 7, column 8, or a homolog or a fragment thereof;
(b) a nucleic acid molecule shown in Table I, application no. 7, column 5 or 8, preferably shown in Table I A, application no. 7, in column 5, or in Table I A, application no. 7, column 8, or in Table I B, application no. 7, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 7, preferably shown in Table II A, application no. 7, in column 5, or in Table II A, application no. 7, column 8, or in Table II B, application no. 7, column 8;
(d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 7, preferably shown in Table I A, application no. 7, in column 5, or in Table I A, application no. 7, column 8, or in Table I B, application no. 7, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 7;
(i) a nucleic acid molecule encoding a polypeptide having the activity represented by a protein as depicted in column 5 of Table II, application no. 7;
(j) a nucleic acid molecule comprising a polynucleotide which is obtained by amplifying a cDNA library or a genomic library using the primers in Table III, application no. 7, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto;
   whereby, preferably, the nucleic acid molecule according to 1 (a), (b), (c), (d), (e), (f), (g), (h), (i), (j) or (k) distinguishes over the sequence depicted in Table I A and/or I B, application no. 7, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 7, column 5 or 8, or the coding region thereof, respectively. In another embodiment, the nucleic acid molecule of the present invention, preferably the coding region thereof, is 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 7, column 5 or 8, or the coding region thereof, respectively. In a further embodiment the nucleic acid molecule does not encode the polypeptide sequence shown in Table II A and/or II B, application no. 7, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 7, column 5 or 8 and does not encode a protein of the sequence shown in Table II A and/or II B, application no. 7, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 7, column 5 and 8, or the coding region thereof. In a further embodiment, the protein of the present invention is at least 30%,40%, 50%, 60%, 70%, 80%, 90%, 95% identical to protein sequence depicted in Table II A and/or II B, application no. 7, column 5 or 8 but less than 100%, preferably less than 99.999%, 99.99% or 99.9%, more preferably less than 99%, 985, 97%, 96% or 95% identical to the sequence shown in Table II A and/or II B, application no. 7, column 5 or 8.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 7.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 7, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 7.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 7, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 7, in the same line as of the respective nucleic acid molecule according to 1) “plastidic” is depicted, or encoding a mitochondrial transit peptide, especially in case in column 6 of Table I, application no. 7, in the same line as of the respective nucleic acid molecule according to 1) “mitochondric” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 7, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 7, in the same line as of the respective nucleic acid molecule according to 1) “plastidic” is depicted, or encoding a mitochondrial transit peptide, especially in case in column 6 of Table I, application no. 7, in the same line as of the respective nucleic acid molecule according to 1) “mitochondric” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 7, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 7, in the same line as of the respective nucleic acid molecule according to 1) “plastidic” is depicted, or encoding a mitochondrial transit peptide, especially in case in column 6 of Table I, application no. 7, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 7.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 7, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 7.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 7, in the same line as of the respective nucleic acid molecule according to 1) “plastidic” is depicted, or encoding a mitochondrial transit peptide, especially in case in column 6 of Table I, application no. 7, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 7, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 7, in the same line as of the respective nucleic acid molecule according to 1) “plastidic” is depicted, or encoding a mitochondrial transit peptide, especially in case in column 6 of Table I, application no. 7, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 7 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 7, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 7 and confers a generation or an increase of the respective fine chemical in a non-human organism or a part thereof as compared to a corresponding wild-type non-human organism or part thereof.

In a preferred embodiment of said vector said nucleic acid molecule according to 1 (a), (b), (c), (d), (e), (f), (g), (h), (i), (j) or (k), said promoter and optionally said nucleic acid molecule encoding a transit peptide are encompassed in an expression cassette. Accordingly in an embodiment the invention provides a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

The invention further provides a vector, especially an expression vector, comprising a FCRP encoding nucleic acid as described above, wherein expression of the vector or FCRP encoding nucleic acid, respectively in a host cell results in the production of a the respective fine chemical, preferably in an increased production of the respective fine chemical, as compared to a wild type of the host cell.

As used herein, the term “vector” refers to a nucleic acid molecule capable of transporting another nucleic acid which has been linked thereto. One type of vector is a “plasmid”, which refers to a circular double stranded DNA loop into which additional DNA segments can be ligated. Another type of vector is a viral vector, wherein additional DNA segments can be ligated into the viral genome. Further types of vectors can be linearized nucleic acid sequences, such as transposons, which are pieces of DNA which can copy and insert themselves. There have been two types of transposons found: simple transposons, known as Insertion Sequences and composite transposons, which can have several genes as well as the genes that are required for transposition. Certain vectors are capable of autonomous replication in a host cell into which they are introduced (e.g. bacterial vectors having a bacterial origin of replication and episomal mammalian vectors). Other vectors (e.g. non-episomal mammalian vectors) are integrated into the genome of a host cell or an organelle upon introduction into the host cell, and thereby are replicated along with the host or organelle genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include other forms of expression vectors, such as viral vectors (e.g., replication defective retroviruses, adenoviruses, and adeno-associated viruses), which exert similar functions.

The term vector furthermore encompasses other vectors known to those skilled in the art such as by way of example phages, viruses such as SV40, CMV, baculovirus, adenovirus, transposons, IS elements, phasmids, phagemids, cosmids, linear or circular DNA.

These vectors can be replicated autonomously in the host organism or be chromosomally replicated, chromosomal replication being preferred.

The recombinant expression vectors of the invention comprise a nucleic acid molecule of the invention in a form suitable for expression of the nucleic acid molecule in a host cell, which means that the recombinant expression vectors include one or more regulatory sequences, selected on the basis of the host cells to be used for expression, and, if desired a targeting sequence, which are operatively linked to the nucleic acid sequence to be expressed. As used herein with respect to a recombinant expression vector, “operatively linked” is intended to mean that the nucleotide sequence of interest is linked to the regulatory sequence(s)/targeting sequence in a manner which allows for expression of the nucleotide sequence in such way that the predicted function assigned to the nucleic sequence is fulfilled (e.g. in an in vitro transcription/translation system or in a host cell when the vector is introduced into the host cell). The term “regulatory sequence” is intended to include promoters, enhancers, and other expression control elements (e.g. polyadenylation signals). Such regulatory sequences are described, for example, in Goeddel “Gene Expression Technology: Methods in Enzymology 185”, Academic Press, San Diego, Calif. (1990), and Gruber and Crosby, in: Methods in Plant Molecular Biology and Biotechnology, eds. Glick and Thompson, Chapter 7, 89-108, CRC Press; Boca Raton, Fla., including the references therein. Regulatory sequences include those that direct constitutive expression of a nucleotide sequence in many types of host cells and those that direct expression of the nucleotide sequence only in certain host cells or under certain conditions. It will be appreciated by those skilled in the art that the design of the expression vector can depend on such factors as the choice of the host cell to be transformed, the level of expression of polypeptide desired, etc. The term “regulatory sequence” is to be considered as being encompassed by the term “regulatory signal”. The expression vectors of the invention can be introduced into host cells to thereby produce polypeptides or peptides, including fusion polypeptides or peptides, encoded by nucleic acid molecules as described herein (e.g., fusion polypeptides, FCRPs etc.).

In another embodiment the recombinant expression vectors of the invention can be designed specifically for expression of the polypeptides of the invention in plant cells. For example, FCRP genes can be expressed in plant cells (see R. Schmidt, L. Willmitzer, Plant Cell Rep. 7, 583 (1988); Plant Molecular Biology and Biotechnology, C Press, Boca Raton, Fla., Chapter 6/7, p. 71-119 (1993); F. F. White, B. Jenes et al., Techniques for Gene Transfer, in: Transgenic Plants, Vol. 1, Engineering and Utilization, eds. Kung and Wu R., 128-43, Academic Press: 1993; Potrykus, Annu. Rev. Plant Physiol. Plant Molec. Biol. 42, 205 (1991) and references cited therein). Suitable host cells are discussed further in Goeddel, “Gene Expression Technology: Methods in Enzymology 185”, Academic Press: San Diego, Calif. (1990). By way of example the plant expression cassette can be installed in the pRT transformation vector ((a) Toepfer et al., Methods Enzymol. 217, 66 (1993), (b) Toepfer et al., Nucl. Acids. Res. 15, 5890 (1987)). Alternatively, the recombinant expression vector can be transcribed and translated in vitro, for example using T7 promoter regulatory sequences and T7 polymerase.

Expression vectors employed in prokaryotes frequently make use of inducible systems with and without fusion proteins or fusion oligopeptides, wherein these fusions can ensue in both N-terminal and C-terminal manner or in other useful domains of a protein. Such fusion vectors usually have the following purposes: 1) to increase the RNA expression rate; 2) to increase the achievable protein synthesis rate; 3) to increase the solubility of the protein; 4) or to simplify purification by means of a binding sequence usable for affinity chromatography. Proteolytic cleavage points are also frequently introduced via fusion proteins, which allow cleavage of a portion of the fusion protein and purification. Such recognition sequences for proteases are recognized, e.g. factor Xa, thrombin and enterokinase.

Typical advantageous fusion and expression vectors are pGEX (Pharmacia Biotech Inc; D. B. Smith and K. S. Johnson, Gene 67, 31 (1988)), pMAL (New England Biolabs, Beverly, Mass.) and pRIT5 (Pharmacia, Piscataway, N.J.) which contains glutathione S-transferase (GST), maltose binding protein or protein A.

In an embodiment of the present invention, the coding sequence for the polypeptide of the invention is cloned into a pGEX expression vector to create a vector encoding a fusion polypeptide comprising, from the N-terminus to the C-terminus, GST-thrombin cleavage site-X polypeptide. The fusion polypeptide can be purified by affinity chromatography using glutathione-agarose resin. Recombinant FCRP unfused to GST can be recovered by cleavage of the fusion polypeptide with thrombin. Other examples of E. coli expression vectors are pTrc (Amann et al., Gene 69, 301 (1988)) and pET vectors (Studier et al., “Gene Expression Technology: Methods in Enzymology 185”, Academic Press, San Diego, Calif. (1990) 60-89; Stratagene, Amsterdam, The Netherlands).

Target gene expression from the pTrc vector relies on host RNA polymerase transcription from a hybrid trp-lac fusion promoter. Target gene expression from the pET 11d vector relies on transcription from a T7 gn10-lac fusion promoter mediated by a co-expressed viral RNA polymerase (T7 gn1). This viral polymerase is supplied by host strains BL21(DE3) or HMS174(DE3) from a resident I prophage harboring a T7 gn1 gene under the transcriptional control of the lacUV 5 promoter.

In a further embodiment of the present invention, the FCRPs are expressed in plants and plant cells such as unicellular plant cells (e.g. algae) (see Falciatore et al., Marine Biotechnology 1 (3), 239 (1999) and references therein) and plant cells from higher plants (e.g., the spermatophytes, such as crop plants), for example to regenerate plants from the plant cells. A nucleic acid molecule coding for FCRP as depicted in Table II, column 5 or 8, or a homolog or a fragment thereof, (and, if desired, in combination with the nucleic acid encoding a respective transit peptide) may be “introduced” into a plant cell by any means, including transfection, transformation or transduction, electroporation, particle bombardment, agroinfection, and the like. One transformation method known to those of skill in the art is the dipping of a flowering plant into an Agrobacteria solution, wherein the Agrobacteria contains the nucleic acid of the invention, followed by breeding of the transformed gametes.

Other suitable methods for transforming or transfecting host cells including plant cells can be found in Sambrook et al., “Molecular Cloning: A Laboratory Manual”. 2nd ed., Cold Spring Harbor Laboratory, Cold Spring Harbor Laboratory Press, Cold Spring Harbor, N.Y., 1989, and other laboratory manuals such as “Methods in Molecular Biology”, 1995, Vol. 44,

Agrobacterium protocols, ed: Gartland and Davey, Humana Press, Totowa, N.J. As generated or increased production of the fine chemical is generally wished to be inherited into a wide variety of monocotyledonous or dicotyledonous plants, preferably plants like maize, wheat, rye, oat, triticale, rice, barley, sugar cane, soybean, peanut, cotton, rapeseed and canola, manihot, pepper, sunflower and tagetes, sugar beet, solanaceous plants like potato, tobacco, eggplant, and tomato, Vicia species, pea, alfalfa, bushy plants (coffee, cacao, tea), Salix species, trees (oil palm, coconut), perennial grasses, and forage crops, these crop plants are also preferred target plants for a genetic engineering as one further embodiment of the present invention. Forage crops include, but are not limited to Wheatgrass, Canarygrass, Bromegrass, Wildrye Grass, Bluegrass, Orchardgrass, Alfalfa, Salfoin, Birdsfoot Trefoil, Alsike Clover, Red Clover and Sweet Clover.

In one embodiment of the present invention, transfection of a nucleic acid molecule coding for FCRP e.g. as depicted in Table II column 5 or 8, or a homolog or a fragment thereof, (and, if desired, in combination with the nucleic acid encoding a respective transit peptide) into a plant is achieved by Agrobacterium mediated gene transfer. Agrobacterium mediated plant transformation can be performed using for example the GV3101(pMP90) (Koncz and Schell, Mol. Gen. Genet. 204, 383 (1986)) or LBA4404 (Clontech) Agrobacterium tumefaciens strain. Transformation can be performed by standard transformation and regeneration techniques (Deblaere et al., Nucl. Acids Res. 13, 4777 (1994), Gelvin, B. Stanton and Robert A. Schilperoort, Plant Molecular Biology Manual, 2nd ed.—Dordrecht : Kluwer Academic Publ., 1995.—in Sect., Ringbuc Zentrale Signatur: BT11-P ISBN 0-7923-2731-4; Bernard R. Glick., John E. Thompson, “Methods in Plant Molecular Biology and Biotechnology”, Boca Raton: CRC Press, 1993 p. 360, ISBN 0-8493-5164-2). For example, rapeseed can be transformed via cotyledon or hypocotyl transformation (Moloney et al., Plant Cell Report 8, 238 (1989); De Block et al., Plant Physiol. 91, 694 (1989)). Use of antibiotics for Agrobacterium and plant selection depends on the binary vector and the Agrobacterium strain used for transformation. Rapeseed selection is normally performed using kanamycin as selectable plant marker. Agrobacterium mediated gene transfer to flax can be performed using, for example, a technique described by Mlynarova et al., Plant Cell Report 13, 282 (1994). Additionally, transformation of soybean can be performed using for example a technique described in European Patent No. 424 047, U.S. Pat. No. 5,322,783, European Patent No. 397 687, U.S. Pat. No. 5,376,543 or U.S. Pat. No. 5,169,770. Transformation of maize can be achieved by particle bombardment, polyethylene glycol mediated DNA uptake or via the silicon carbide fiber technique. (See, for example, Freeling and Walbot “The maize handbook” Springer Verlag: New York (1993) ISBN 3-540. 97826-7). A specific example of maize transformation is found in U.S. Pat. No. 5,990,387, and a specific example of wheat transformation can be found in PCT Application No. WO 93/07256. Examples for rice transformation can be found e.g. in EP 1 728 418 or EP 897 013.

According to the present invention, the introduced nucleic acid molecule coding for FCRP e.g. as depicted in Table II, column 5 or 8, or a homolog or a fragment thereof, may be maintained in the plant cell stably if it is incorporated into a non-chromosomal autonomous replicon or integrated into the plant chromosomes or organelle genome. Alternatively, the introduced FCRP may be present on an extra-chromosomal non-replicating vector and be transiently expressed or transiently active.

Whether present in an extra-chromosomal non-replicating vector or a vector that is integrated into a chromosome, the nucleic acid molecule coding for FCRP as depicted in Table II, column 5 or 8, a homolog or a fragment thereof, preferably resides in a non-human organism expression cassette, preferably a microorganism expression cassette or a plant expression cassette. A plant expression cassette preferably contains regulatory sequences capable of driving gene expression in plant cells that are operatively linked so that each sequence can fulfill its function, for example, termination of transcription by polyadenylation signals. Preferred polyadenylation signals are those originating from Agrobacterium tumefaciens t-DNA such as the gene 3 known as octopine synthase of the Ti-plasmid pTiACH5 (Gielen et al., EMBO J. 3, 835 (1984)) or functional equivalents thereof but also all other terminators functionally active in plants are suitable. As plant gene expression is very often not limited on transcriptional levels, a plant expression cassette preferably contains other operatively linked sequences like translational enhancers such as the overdrive-sequence containing the 5″-untranslated leader sequence from tobacco mosaic virus enhancing the polypeptide per RNA ratio (Gallie et al., Nucl. Acids Research 15, 8693 (1987)). Examples of plant expression vectors include those detailed in: D. Becker et al., Plant Mol. Biol. 20, 1195 (1992); and M. W. Bevan, Nucl. Acid. Res. 12, 8711 (1984); and “Vectors for Gene Transfer in Higher Plants” in: Transgenic Plants, Vol. 1, Engineering and Utilization, eds. Kung and Wu R., Academic Press, 1993, S. 15-38. “Transformation” is defined herein as a process for introducing DNA into a non-human organism, preferably a microorganism or a plant or a part thereof, like a plant cell or plant tissue. In an embodiment the transformation is performed with heterologous DNA. In another embodiment the transformation is performed with “additional” homologous DNA. Transformation may occur under natural or artificial conditions using various methods well known in the art. Transformation may rely on any known method for the insertion of (foreign, additional) nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome. They also include cells which transiently express the inserted DNA or RNA for limited periods of time. Transformed plant cells, plant tissue, or plants are understood to encompass not only the end product of a transformation process, but also transgenic progeny thereof.

The terms “transformed,” “transgenic,” and “recombinant” refer to a host organism such as a microorganism, e.g. a bacterium, or a plant into which a nucleic acid molecule has been introduced. In an embodiment the introduced nucleic acid molecule is heterogenous. In another embodiment the introduced DNA is homologous. The nucleic acid molecule can be stably integrated into the genome of the host or the nucleic acid molecule can also be present as an extra-chromosomal molecule. Such an extra-chromosomal molecule can be autoreplicating. A “non-transformed”, “non-transgenic” or “non-recombinant” host refers to a wild-type organism, e.g. a bacterium or plant, which does not contain the heterologous nucleic acid molecule or the additional homologous nucleic acid molecule.

In an embodiment a “transgenic plant”, as used herein, refers to a plant which contains a nucleotide sequence inserted into either its nuclear genome or organelle genome. In an embodiment the introduced nucleic acid molecule is heterogenous. In another embodiment the introduced DNA is homologous. In each case it encompasses further the offspring generations i.e. the Ti-, T2- and consecutively generations or BC1-, BC2- and consecutively generation as well as crossbreeds thereof with non-transgenic or other transgenic plants, as long as it contains said nucleic acid sequence.

The host organism (=transgenic organism) advantageously contains at least one copy of the nucleic acid in addition according to the invention and/or of the nucleic acid construct in addition according to the invention.

In principle all non-human organism can be used as host organism. In an embodiment the transgenic non-human organism or cell is a prokaryotic organism. In an embodiment the transgenic non-human organism or cell is an eukaryotic organism, like an alga, a non-human animal or a plant, in particular an algae or a plant. Preferred transgenic plants are monocotyledonous or dicotyledonous plants, preferably, for example, selected from the families Aceraceae, Anacardiaceae, Apiaceae, Asteraceae, Brassicaceae, Cactaceae, Cucurbitaceae, Euphorbiaceae, Fabaceae, Malvaceae, Nymphaeaceae, Papaveraceae, Rosaceae, Salicaceae, Solanaceae, Arecaceae, Bromeliaceae, Cyperaceae, lridaceae, Liliaceae, Orchidaceae, Gentianaceae, Labiaceae, Magnoliaceae, Ranunculaceae, Carifolaceae, Rubiaceae, Scrophulariaceae, Caryophyllaceae, Ericaceae, Polygonaceae, Violaceae, Juncaceae or Poaceae and preferably from a plant selected from the group of the families Apiaceae, Asteraceae, Brassicaceae, Cucurbitaceae, Fabaceae, Papaveraceae, Rosaceae, Solanaceae, Liliaceae or Poaceae. Preferred are crop plants such as plants advantageously selected from the group of the genus peanut, oilseed rape, canola, sunflower, safflower, olive, sesame, hazelnut, almond, avocado, bay, pumpkin/squash, linseed, soya, pistachio, borage, maize, wheat, rye, oats, sorghum and millet, triticale, rice, barley, sugarcane, cotton, cassava, potato, sugarbeet, egg plant, alfalfa, and perennial grasses and forage plants, oil palm, vegetables (brassicas, root vegetables, tuber vegetables, pod vegetables, fruiting vegetables, onion vegetables, leafy vegetables and stem vegetables), buckwheat, Jerusalem artichoke, broad bean, vetches, lentil, dwarf bean, lupin, clover and lucerne for mentioning only some of them.

In one embodiment of the invention transgenic plants are selected from the group comprising monocotyledonous or dicotyledonous plants, preferably cereals, soybean, rapeseed (including oil seed rape, especially canola and winter oil seed rape), cotton, sugarcane and potato, especially corn, soy, rapeseed (including oil seed rape, especially canola and winter oil seed rape), cotton, wheat and rice.

In one embodiment, the transgenic plant is a crop plant, in particular a transgenic plant belonging to e.g. Anacardium occidentale, Calendula officinalis, Carthamus tinctorius, Cichorium intybus, Cynara scolymus, Helianthus annus, Tagetes lucida, Tagetes erecta, Tagetes tenuifolia; Daucus carota; Corylus avellana, Corylus colurna, Borago officinalis; Brassica napus, Brassica rapa ssp., Sinapis arvensis Brassica juncea, Brassica juncea var. juncea, Brassica juncea var. crispifolia, Brassica juncea var. foliosa, Brassica nigra, Brassica sinapioides, Melanosinapis communis, Brassica oleracea, Arabidopsis thaliana, Anana comosus, Ananas ananas, Bromelia comosa, Carica papaya, Cannabis sative, Ipomoea batatus, Ipomoea pandurata, Convolvulus batatas, Convolvulus tiliaceus, Ipomoea fastigiata, Ipomoea tiliacea, Ipomoea triloba, Convolvulus panduratus, Beta vulgaris, Beta vulgaris var. altissima, Beta vulgaris var. vulgaris, Beta maritima, Beta vulgaris var. perennis, Beta vulgaris var. conditiva, Beta vulgaris var. esculenta, Cucurbita maxima, Cucurbita mixta, Cucurbita pepo, Cucurbita moschata, Olea europaea, Manihot utilissima, Janipha manihot, Jatropha manihot, Manihot aipil, Manihot dulcis, Manihot manihot, Manihot melanobasis, Manihot esculenta, Ricinus communis, Pisum sativum, Pisum arvense, Pisum humile, Medicago sativa, Medicago falcata, Medicago varia, Glycine max Dolichos soja, Glycine gracilis, Glycine hispida, Phaseolus max, Soja hispida, Glycine max, Cocos nucifera, Pelargonium grossularioides, Oleum cocoas, Laurus nobilis, Persea americana, Arachis hypogaea, Linum usitatissimum, Linum humile, Linum austriacum, Linum bienne, Linum angustifolium, Linum catharticum, Linum flavum, Linum grandiflorum, Adenolinum grandiflorum, Linum lewisii, Linum narbonense, Linum perenne, Linum perenne var. lewisii, Linum pratense, Linum trigynum, Punica granatum, Gossypium hirsutum, Gossypium arboreum, Gossypium barbadense, Gossypium herbaceum, Gossypium thurberi, Musa nana, Musa acuminata, Musa paradisiaca, Musa spp., Elaeis guineensis, Papaver orientale, Papaver rhoeas, Papaver dubium, Sesamum indicum, Piper aduncum, Piper amalago, Piper angustifolium, Piper auritum, Piper betel, Piper cubeba, Piper longum, Piper nigrum, Piper retrofractum, Artanthe adunca, Artanthe elongata, Peperomia elongata, Piper elongatum, Steffensia elongata, Hordeum vulgare, Hordeum jubatum, Hordeum murinum, Hordeum secalinum, Hordeum distichon Hordeum aegiceras, Hordeum hexastichon., Hordeum hexastichum, Hordeum irregulare, Hordeum sativum, Hordeum secalinum, Avena sativa, Avena fatua, Avena byzantina, Avena fatua var. sativa, Avena hybrida, Sorghum bicolor, Sorghum halepense, Sorghum saccharatum, Sorghum vulgare, Andropogon drummondii, Holcus bicolor, Holcus sorghum, Sorghum aethiopicum, Sorghum arundinaceum, Sorghum caffrorum, Sorghum cernuum, Sorghum dochna, Sorghum drummondii, Sorghum durra, Sorghum guineense, Sorghum lanceolatum, Sorghum nervosum, Sorghum saccharatum, Sorghum subglabrescens, Sorghum verticilliflorum, Sorghum vulgare, Holcus halepensis, Sorghum miliaceum millet, Panicum militaceum, Zea mays, Triticum aestivum, Triticum durum, Triticum turgidum, Triticum hybernum, Triticum macha, Triticum sativum or Triticum vulgare, Cofea spp., Coffea arabica, Coffea canephora, Coffea liberica, Capsicum annuum, Capsicum annuum var. glabriusculum, Capsicum frutescens, Capsicum annuum, Nicotiana tabacum, Solanum tuberosum, Solanum melongena, Lycopersicon esculentum, Lycopersicon lycopersicum, Lycopersicon pyriforme, Solanum integrifolium, Solanum lycopersicum Theobroma cacao, Panicum virgatum or Camellia sinensis.

In another embodiment, the transgenic plant belongs to monocotyledonous or dicotyledonous plants, preferably to Anacardiaceae such as the genera Pistacia, Mangifera, Anacardium e.g. the species Pistacia vera [pistachios, Pistazie], Mangifer indica [Mango] or Anacardium occidentale [Cashew]; Asteraceae such as the genera Calendula, Carthamus, Centaurea, Cichorium, Cynara, Helianthus, Lactuca, Locusta, Tagetes, Valeriana e.g. the species Calendula officinalis [Marigold], Carthamus tinctorius [safflower], Centaurea cyanus [cornflower], Cichorium intybus [blue daisy], Cynara scolymus [Artichoke], Helianthus annus [sunflower], Lactuca sativa, Lactuca crispa, Lactuca esculenta, Lactuca scariola L. ssp. sativa, Lactuca scariola L. var. integrate, Lactuca scariola L. var. integrifolia, Lactuca sativa subsp. romana, Locusta communis, Valeriana locusta [lettuce], Tagetes lucida, Tagetes erecta or Tagetes tenuifolia [Marigold]; Apiaceae such as the genera Daucus e.g. the species Daucus carota [carrot]; Betulaceae such as the genera Corylus e.g. the species Corylus avellana or Corylus colurna [hazelnut]; Boraginaceae such as the genera Borago e.g. the species Borago officinalis [borage]; Brassicaceae such as the genera Brassica, Melanosinapis, Sinapis, Arabadopsis e.g. the species Brassica napus, Brassica rapa ssp. [canola, oilseed rape, turnip rape], Sinapis arvensis Brassica juncea, Brassica juncea var. juncea, Brassica juncea var. crispifolia, Brassica juncea var. foliosa, Brassica nigra, Brassica sinapioides, Melanosinapis communis [mustard], Brassica oleracea [fodder beet] or Arabidopsis thaliana; Bromeliaceae such as the genera Anana, Bromelia e.g. the species Anana comosus, Ananas ananas or Bromelia comosa [pineapple]; Caricaceae such as the genera Carica e.g. the species Carica papaya [papaya]; Cannabaceae such as the genera Cannabis e.g. the species Cannabis sative [hemp], Convolvulaceae such as the genera Ipomea, Convolvulus e.g. the species Ipomoea batatus, Ipomoea pandurata, Convolvulus batatas, Convolvulus tiliaceus, Ipomoea fastigiata, Ipomoea tiliacea, Ipomoea triloba or Convolvulus panduratus [sweet potato, Man of the Earth, wild potato], Chenopodiaceae such as the genera Beta, i.e. the species Beta vulgaris, Beta vulgaris var. altissima, Beta vulgaris var. Vulgaris, Beta maritima, Beta vulgaris var. perennis, Beta vulgaris var. conditiva or Beta vulgaris var. esculenta [sugar beet]; Cucurbitaceae such as the genera Cucubita e.g. the species Cucurbita maxima, Cucurbita mixta, Cucurbita pepo or Cucurbita moschata [pumpkin, squash]; Elaeagnaceae such as the genera Elaeagnus e.g. the species Olea europaea [olive]; Ericaceae such as the genera Kalmia e.g. the species Kalmia latifolia, Kalmia angustifolia, Kalmia microphylla, Kalmia polifolia, Kalmia occidentalis, Cistus chamaerhodendros or Kalmia lucida [American laurel, broad-leafed laurel, calico bush, spoon wood, sheep laurel, alpine laurel, bog laurel, western bog-laurel, swamp-laurel]; Euphorbiaceae such as the genera Manihot, Janipha, Jatropha, Ricinus e.g. the species Manihot utilissima, Janipha manihot, Jatropha manihot, Manihot aipil, Manihot dulcis, Manihot manihot, Manihot melanobasis, Manihot esculenta [manihot, arrowroot, tapioca, cassava] or Ricinus communis [castor bean, Castor Oil Bush, Castor Oil Plant, Palma Christi, Wonder Tree]; Fabaceae such as the genera Pisum, Albizia, Cathormion, Feuillea, Inga, Pithecolobium, Acacia, Mimosa, Medicajo, Glycine, Dolichos, Phaseolus, Soja e.g. the species Pisum sativum, Pisum arvense, Pisum humile [pea], Albizia berteriana, Albizia julibrissin, Albizia lebbeck, Acacia berteriana, Acacia littoralis, Albizia berteriana, Albizzia berteriana, Cathormion berteriana, Feuillea berteriana, Inga fragrans, Pithecellobium berterianum, Pithecellobium fragrans, Pithecolobium berterianum, Pseudalbizzia berteriana, Acacia julibrissin, Acacia nemu, Albizia nemu, Feuilleea julibrissin, Mimosa julibrissin, Mimosa speciosa, Sericanrda julibrissin, Acacia lebbeck, Acacia macrophylla, Albizia lebbek, Feuilleea lebbeck, Mimosa lebbeck, Mimosa speciosa [bastard logwood, silk tree, East Indian Walnut], Medicago sativa, Medicago falcata, Medicago varia [alfalfa], Glycine max [soybean ], Dolichos soja, Glycine gracilis, Glycine hispida, Phaseolus max or Soja hispida; Geraniaceae such as the genera Pelargonium, Cocos, Oleum e.g. the species Cocos nucifera, Pelargonium grossularioides or Oleum cocois [coconut]; Gramineae such as the genera Saccharum e.g. the species Saccharum officinarum; Juglandaceae such as the genera Juglans, Wallia e.g. the species Juglans regia, Juglans ailanthifolia, Juglans sieboldiana, Juglans cinerea, Wallia cinerea, Juglans bixbyi, Juglans californica, Juglans hindsii, Juglans intermedia, Juglans jamaicensis, Juglans major, Juglans microcarpa, Juglans nigra or Wallia nigra [walnut, black walnut, common walnut, persian walnut, white walnut, butternut, black walnut]; Lauraceae such as the genera Persea, Laurus e.g. the species laurel Laurus nobilis [bay, laurel, bay laurel, sweet bay], Persea americana Persea americana, Persea gratissima or Persea persea [avocado]; Leguminosae such as the genera Arachis e.g. the species Arachis hypogaea [peanut]; Linaceae such as the genera Linum, Adenolinum e.g. the species Linum usitatissimum, Linum humile, Linum austriacum, Linum bienne, Linum angustifolium, Linum catharticum, Linum flavum, Linum grandiflorum, Adenolinum grandiflorum, Linum lewisii, Linum narbonense, Linum perenne, Linum perenne var. lewisii, Linum pratense or Linum trigynum [flax, linseed]; Lythrarieae such as the genera Punica e.g. the species Punica granatum [pomegranate]; Malvaceae such as the genera Gossypium e.g. the species Gossypium hirsutum, Gossypium arboreum, Gossypium barbadense, Gossypium herbaceum or Gossypium thurberi [cotton]; Musaceae such as the genera Musa e.g. the species Musa nana, Musa acuminata, Musa paradisiaca, Musa spp. [banana ]; Onagraceae such as the genera Camissonia, Oenothera e.g. the species Oenothera biennis or Camissonia brevipes [primrose, evening primrose]; Palmae such as the genera Elacis e.g. the species Elaeis guineensis [oil plam]; Papaveraceae such as the genera Papaver e.g. the species Papaver orientale, Papaver rhoeas, Papaver dubium [poppy, oriental poppy, corn poppy, field poppy, shirley poppies, field poppy, long-headed poppy, long-pod poppy]; Pedaliaceae such as the genera Sesamum e.g. the species Sesamum indicum [sesame]; Piperaceae such as the genera Piper, Artanthe, Peperomia, Steffensia e.g. the species Piper aduncum, Piper amalago, Piper angustifolium, Piper auritum, Piper betel, Piper cubeba, Piper longum, Piper nigrum, Piper retrofractum, Artanthe adunca, Artanthe elongata, Peperomia elongata, Piper elongatum, Steffensia elongata. [Cayenne pepper, wild pepper]; Poaceae such as the genera Hordeum, Secale, Avena, Sorghum, Andropogon, Holcus, Panicum, Oryza, Zea, Triticum e.g. the species Hordeum vulgare, Hordeum jubatum, Hordeum murinum, Hordeum secalinum, Hordeum distichon Hordeum aegiceras, Hordeum hexastichon, Hordeum hexastichum, Hordeum irregulare, Hordeum sativum, Hordeum secalinum [barley, pearl barley, foxtail barley, wall barley, meadow barley], Secale cereale [rye], Avena sativa, Avena fatua, Avena byzantina, Avena fatua var. sativa, Avena hybrida [oat], Sorghum bicolor, Sorghum halepense, Sorghum saccharatum, Sorghum vulgare, Andropogon drummondii, Holcus bicolor, Holcus sorghum, Sorghum aethiopicum, Sorghum arundinaceum, Sorghum caffrorum, Sorghum cernuum, Sorghum dochna, Sorghum drummondii, Sorghum durra, Sorghum guineense, Sorghum lanceolatum, Sorghum nervosum, Sorghum saccharatum, Sorghum subglabrescens, Sorghum verticilliflorum, Sorghum vulgare, Holcus halepensis, Sorghum miliaceum millet, Panicum militaceum [Sorghum, millet], Oryza sativa, Oryza latifolia [rice], Zea mays [corn, maize] Triticum aestivum, Triticum durum, Triticum turgidum, Triticum hybernum, Triticum macha, Triticum sativum or Triticum vulgare [wheat, bread wheat, common wheat], Proteaceae such as the genera Macadamia e.g. the species Macadamia intergrifolia [macadamia]; Rubiaceae such as the genera Coffea e.g. the species Cofea spp., Coffea arabica, Coffea canephora or Coffea liberica [coffee]; Scrophulariaceae such as the genera Verbascum e.g. the species Verbascum blattaria, Verbascum chaixii, Verbascum densiflorum, Verbascum lagurus, Verbascum longifolium, Verbascum lychnitis, Verbascum nigrum, Verbascum olympicum, Verbascum phlomoides, Verbascum phoenicum, Verbascum pulverulentum or Verbascum thapsus [mullein, white moth mullein, nettle-leaved mullein, dense-flowered mullein, silver mullein, long-leaved mullein, white mullein, dark mullein, greek mullein, orange mullein, purple mullein, hoary mullein, great mullein]; Solanaceae such as the genera Capsicum, Nicotiana, Solanum, Lycopersicon e.g. the species Capsicum annuum, Capsicum annuum var. glabriusculum, Capsicum frutescens [pepper], Capsicum annuum [paprika], Nicotiana tabacum, Nicotiana alata, Nicotiana attenuata, Nicotiana glauca, Nicotiana langsdorffii, Nicotiana obtusifolia, Nicotiana quadrivalvis, Nicotiana repanda, Nicotiana rustica, Nicotiana sylvestris [tobacco], Solanum tuberosum [potato], Solanum melongena [egg-plant] (Lycopersicon esculentum, Lycopersicon lycopersicum, Lycopersicon pyriforme, Solanum integrifolium or Solanum lycopersicum [tomato]; Sterculiaceae such as the genera Theobroma e.g. the species Theobroma cacao [cacao]; Theaceae such as the genera Camellia e.g. the species Camellia sinensis) [tea].

In another embodiment of the invention the transgenic non-human organism is a transgenic microorganism like selected from the group consisting of Charophyceae such as the genera Chara, Nitella e.g. the species Chara globularis, Chara vulgaris, Nitella flexilis, Chlorophyceae such as the genera Acrosiphonia, Spongomorpha, Urospora, Bryopsis, Pseudobryopsis, Trichosolen, Dichotomosiphon, Caulerpa, Rhipilia, Blastophysa, Avrainvillea, Chlorodesmis, Codium, Espera, Halicystis, Halimeda, Penicillus, Pseudocodium, Rhipiliopsis, Rhipocephalus, Tydemania, Udotea, Derbesia, Acrochaete, Aphanochaete, Bolbocoleon, Chaetobolus, Chaetonema, Chaetophora, Chlorotylium, Desmococcus, Draparnaldia, Draparnaldiopsis, Ectochaete, Endophyton, Entocladia, Epicladia, Internoretia, Microthamnion, Ochlochaete, Phaeophila, Pilinella, Pringsheimiella, Protoderma, Pseudendoclonium, Pseudodictyon, Pseudopringsheimia, Pseudulvella, Schizomeris, Stigeoclonium, Thamniochaete, Ulvella, Pilinia, Tellamia, Helicodictyon, Actidesmium, Ankyra, Characium, Codiolum, Sykidion, Keratococcus, Prototheca, Bracteacoccus Chlorococcum, Excentrosphaera, Hormidium, Oophila, Schroederia, Tetraedron, Trebouxia Chlorosarcinopsis, Gomphonitzschia, Coccomyxa, Dactylothece, Diógenes, Disporá, Gloeocystis, Mycanthococcus, Ourococcus, Coelastrum, Dicranochaete, Botryococcus, Dictyosphaerium, Dimorphococcus, Chlorochytrium, Kentrosphaera, Phyllobium, Gomontia, Hormotila, Euastropsis, Hydrodictyon, Pectodictyon, Pediastrum, Sorastrum, Tetrapedia, Acanthosphaera, Echinosphaerella, Echinosphaeridium, Errerella, Gloeoactinium, Golenkeniopsis Golenkinia, Micractinium, Ankistrodesmus, Chlorella, Chodatella, Closteriopsis, Cryocystis, Dactylococcus, Dematractum, Eremosphaera, Eutetramorus, Franceia, Glaucocystis, Gloeotaenium, Kirchneriella, Lagerheimiella, Monoraphidium, Nannochloris, Nephrochlamys, Nephrocytium, Oocystis, Oonephris, Pachycladon, Palmellococcus, Planktosphaeria, Polyedriopsis, Pseudoraciborskia, Quadrigula, Radiococcus, Rochiscia, Scotiella, Selanastrum, Thorakochloris, Treubaria, Trochiscia, Westella, Zoochlorella, Ostreobium, Phyllosiphon, Protosiphon, Rhodochytrium, Actinastrum, Coronastrum, Crucigenia, Dictymocystis, Enallax, Scenedesmus, Selenastrum, Tetradesmus, Tetrallantos, Tetrastrum, Chlorosarcina, Anadyomene, Valoniopsis, Ventricaria, Basicladia, Chaetomorpha, Cladophora, LolaPithophoraRhizoclonium Chaetosphaeridium, Conochaete, Coleochaete, Oligochaetophora, Polychaetophora, Cylindrocapsa, Gongrosira, Protococcus, Acetabularia, Batophora, Bornetella, Dasycladus, Halicoryne, Neomeris, Elakatothrix, Raphidonema, Microspora, Bulbochaete, Oedocladium, Oedogonium, Prasiola, Rosenvingiella, Schizogonium, Apjohnia, Chamaedoris, Cladophoropsis, Siphonocladus, Spongocladia, Boergesenia, Boodlea, Cystodictyon, Dictyosphaeria, Ernodesmis, Microdictyon, Struvea, Valonia, Sphaeroplea, Malleochloris, Stylosphaeridium, Gloeococcus, Palmella, Palmodictyon, Palmophyllum, Pseudospherocystis, Sphaerocystis, Urococcus, Apiocystis, Chaetopeltis, Gemellicystis, Paulschulzia, Phacomyxa, Pseudotetraspora, Schizochlamys, Tetraspora, Cephaleuros, Ctenocladus, Epibolium, Leptosira, Trentepohlia, Diplochaete, Monostroma, Binuclearia, Geminella, Klebsormidium, Planetonema, Radiofilum, Stichococcus, Ulothrix, Uronema, Blidingia, Capsosiphon, Chloropelta, Enteromorpha, Percursaria, Ulva, Ulvaria, Brachiomonas, Carteria, Chlainomonas, Chlamydomonas, Chlamydonephris, Chlorangium, Chlorogonium, Cyanidium, Fortiella, Glenomonas, Gloeomonas, Hyalogonium, Lobomonas Polytoma, Pyramichlamys, Scourfieldia, Smithsonimonas, Sphaerellopsis, Sphenochloris, Spirogonium, Collodictyon, Dunaliella, Haematococcus, Stephanosphaera, Coccomonas, Dysmorphococcus, Phacotus, Pteromonas, Thoracomonas, Wislouchiella, Mascherina, Pyrobotrys, Spondylomorum, Eudorina, Gonium, Oltmannsiella, Pandorina, Platydorina, Pleodorina, Stephanoon, Volvox, Volvulina, Actinotaenium, Arthrodesmus, Bambusina Closterium, Cosmarium, Desmidium, Euastrum, Groenbladia, Hyalotheca, Micrasterias, Penium, Phymatodocis, Pleurotaenium, Sphaerozosma, Spinoclosterium, Spinocosmarium, Spondylosium, Staurastrum, Tetmemorus, Triploceras, Xanthidium, Cylindrocystis, Genicularia, Gonatozygon, Mesotaenium, Netrium, Roya, Spirotaenia, Cosmocladium, Debarya, Docidium, Euastridium, Hallasia, Mougeotia, Mougeotiopsis, Sirogonium, Spirogyra, Staurodesmus, Teilingia, Zygnema, Zygogonium, e.g. the species Caulerpa taxifolia, Prototheca wickerhamii, Ankistrodesmus falcatus, Chlorella ellipsoidea, Chlorella pyrenoidosa, Clorella sorokiniana, Chlorella vulgaris, Scenedesmus obliquus, Scenedesmus quadricauda, Selenastrum capricornutum, Selenastrum undecimnotata, Cladophora glomerata, Chlamydomonas eugametos, Chlamydomonas reinhardtii, Cyanidium caldarium, Dunaliella salina, Dunaliella tertiolecta, Euglena gracilis, Haematococcus pluvialis, Coniugatophyceae, Prasinophyceae Trebouxiophyceae, Ulvophyceae, Chlorodendraceae, Pedinomonadales, Halosphaeraceae, Pterospermataceae, Monomastigaceae, Pyramimonadaceae, Chlorodendraceae such as the genera Prasinocladus e.g. the species Prasinocladus ascus, Halosphaeraceae, Pedinomonadales, Pedinomonadaceae such as the genera Pedinomonas, Pterospermataceae such as the genera Pachysphaera, Pterosperma, Halosphaera, Pyramimonas, Bacillariophyceae, Chrysophyceae, Craspedophyceae, Euglenophyceae, Prymnesiophyceae, Phaeophyceae, Dinophyceae, Rhodophyceae, Xanthophyceae, Prasinophyceae such as the genera Nephroselmis, Prasinococcus, Scherffelia, Tetraselmis, Mantoniella, Ostreococcus e.g. the species Nephroselmis olivacea, Prasinococcus capsulatus, Scherffelia dubia, Tetraselmis chui, Tetraselmis suecica, Mantoniella squamata or Ostreococcus tauri.

Other useful organism are prokaryotic host organism, which may be useful for the cloning of the desired nucleic acid constructs or vectors such as the genera Escherichia, for example the species Escherichia coli, in particular E. coli K12 and its described strains or Agrobacterium, for example Agrobacterium tumefaciens.

The introduction of the nucleic acids according to the invention, the expression cassette or the vector into the non-human organism, like a microorganism or a plant, can in principle be done by all of the methods known to those skilled in the art. The introduction of the nucleic acid sequences gives rise to recombinant or transgenic organisms.

After the introduction of the nucleic acid, nucleic acid construct, expression cassette or vector, and the expression thereof the transgenic organism or cell is advantageously cultured and subsequently harvested.

Unless otherwise specified, the terms “polynucleotides”, “nucleic acid” and “nucleic acid molecule” as used herein are interchangeably. Unless otherwise specified, the terms “peptide”, “polypeptide” and “protein” are interchangeably in the present context. The term “sequence” may relate to polynucleotides, nucleic acids, nucleic acid molecules, peptides, polypeptides and proteins, depending on the context in which the term “sequence” is used. The terms “gene(s)”, “polynucleotide”, “nucleic acid sequence”, “nucleotide sequence”, or “nucleic acid molecule(s)” as used herein refers to a polymeric form of nucleotides of any length, either ribonucleotides or deoxyribonucleotides. The terms refer only to the primary structure of the molecule.

Thus, the terms “gene(s)”, “polynucleotide”, “nucleic acid sequence”, “nucleotide sequence”, or “nucleic acid molecule(s)” as used herein include double- and single-stranded DNA and RNA. They also include known types of modifications, for example, methylation, “caps”, substitutions of one or more of the naturally occurring nucleotides with an analog. Preferably, the DNA or RNA sequence of the invention comprises a coding sequence encoding the herein defined polypeptide.

The genes of the invention, coding in one embodiment for an activity selected from the group consisting of 3-deoxy-7-phosphoheptulonate synthase, 3-deoxy-D-arabinoheptulosonate-7-phosphatesynthase, ABC transporter permease subunit, acetyltransferase, acid shock protein, adenylate kinase, adenylosuccinate lyase, amino-acid acetyltransferase, ankyrin repeat family protein, At1g29350-protein, ATP-binding component of a transport system, b1163-protein, b1219-protein, b1234-protein, b1522-protein, b1921-protein, b2360-protein, b2548-protein, b2613-protein, b2673-protein, b2812-protein, b3890-protein, b3989-protein, b4029-protein, b4050-protein, b4056-protein, calcium-dependent protein kinase, carbohydrate kinase, colanic acid biosynthesis protein, coproporphyrinogen oxidase, cyclin D , DNA polymerase subunit, electron transfer flavoprotein subunit beta, geranylgeranyl pyrophosphate synthase, geranyltranstransferase, gluconate transport system permease 3, glucose dehydrogenase, glucose-6-phosphate 1-dehydrogenase, glutaredoxin, glycerol dehydrogenase, glycerol-3-phosphate dehydrogenase, glycolate utilization protein, harpin-induced family protein, homocitrate synthase, hydrolase, isopropylmalate isomerase large subunit, lipopolysaccharide biosynthesis protein, MADS box protein transcription factor, major facilitator superfamily transporter protein, malate dehydrogenase, monothiol glutaredoxin, monthiol glutaredoxin, multiple antibiotic resistance protein, murein transglycosylase, N-(5′-phosphoribosyl)anthranilate isomerase, NAD-dependent epimerase/dehydratase, NADP-dependent malic enzyme, O-antigen chain length determinant, phosphatidylserine decarboxylase, phospho-2-dehydro-3-deoxyheptonate aldolase, phosphoadenosine phosphosulfate reductase , phosphofructokinase, photosystem II protein, polygalacturonase, polyphosphate kinase, precorrin methylase, protein kinase, protein phosphatase, putative transport system permease protein, pyruvate kinase, RNA-binding protein, Sec-independent protein translocase subunit, serine protease, sll1761-protein, slr1917-protein, small nuclear ribonucleoprotein , ThiF family protein, thioredoxin, thioredoxin family protein, threonine synthase, transcription factor, transcriptional regulator, transport protein, trehalose-phosphatase, TTC1386-protein, ycl049c-protein, ydl235c-protein, ydr183w-protein, yer106w-protein, ylr178c-protein, ypr170c-protein, and zinc finger protein are also called “FCRP genes”.

A “coding sequence” is a nucleotide sequence, which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. The triplets taa, tga and tag represent the (usual) stop codons which are interchangeable. A coding sequence can include, but is not limited to mRNA, cDNA, recombinant nucleotide sequences or genomic DNA, while introns may be present as well under certain circumstances.

To introduce a nucleic acid molecule into a nucleic acid construct, e.g. as part of an expression cassette, the codogenic gene segment is advantageously subjected to an amplification and ligation reaction in the manner known by a skilled person. It is preferred to follow a procedure similar to the protocol for the Pfu DNA polymerase or a Pfu/Taq DNA polymerase mixture. The primers are selected according to the sequence to be amplified. The primers should expediently be chosen in such a way that the amplificate comprise the codogenic sequence from the start to the stop codon. After the amplification, the amplificate is expediently analyzed. For example, the analysis may consider quality and quantity and be carried out following separation by gel electrophoresis. Thereafter, the amplificate can be purified following a standard protocol (for example Qiagen). An aliquot of the purified amplificate is then available for the subsequent cloning step. The skilled worker generally knows suitable cloning vectors.

They include, in particular, vectors which are capable of replication in easy to handle cloning systems like as bacterial, yeast or insect cell based (e.g. baculovirus expression) systems, that is to say especially vectors which ensure efficient cloning in E. coli, and which make possible the stable transformation of plants. Vectors, which must be mentioned, in particular are various binary and cointegrated vector systems, which are suitable for the T-DNA-mediated transformation. Such vector systems are generally characterized in that they contain at least the vir genes, which are required for the Agrobacterium-mediated transformation, and the T-DNA border sequences.

In general, vector systems preferably also comprise further cisregulatory regions such as promoters and terminators and/or selection markers by means of which suitably transformed organisms can be identified. While vir genes and T-DNA sequences are located on the same vector in the case of cointegrated vector systems, binary systems are based on at least two vectors, one of which bears vir genes, but no T-DNA, while a second one bears T-DNA, but no vir gene. Owing to this fact, the last-mentioned vectors are relatively small, easy to manipulate and capable of replication in E. coli and in Agrobacterium. These binary vectors include vectors from the series pBIB-HYG, pPZP, pBecks, pGreen. Those which are preferably used in accordance with the invention are Bin19, pBI101, pBinAR, pGPTV and pCAMBIA. An overview of binary vectors and their use is given by Hellens et al, Trends in Plant Science 5, 446-451 (2000). In case of a targeted expression the vectors are preferably modified in such a manner, that they already contain the nucleic acid coding for the transit peptide and that the nuleic acids of the invention, preferentially the nucleic acid sequences encoding the polypeptides shown in the respective line in Table II, column 5 or 8, or a homolog or a fragment thereof, can be cloned 3″prime to the transit peptide encoding sequence, leading to a functional preprotein, which is directed to the intended compartment, like plastids or mitochondria, and which means that the mature protein fulfills its biological activity.

For a vector preparation, vectors may first be linearized using restriction endonuclease(s) and then be modified enzymatically in a suitable manner. Thereafter, the vector is purified, and an aliquot is employed in the cloning step. In the cloning step, the enzymecleaved and, if required, purified amplificate is cloned together with similarly prepared vector fragments, using ligase. In this context, a specific nucleic acid construct, or vector or plasmid construct, may have one or else more codogenic gene segments. The codogenic gene segments in these constructs are preferably linked operably to regulatory sequences. The regulatory sequences include, in particular, plant sequences like the above-described promoters and terminators. The constructs can advantageously be propagated stably in microorganisms, in particular Escherichia coli and/or Agrobacterium tumefaciens, under selective conditions and enable the transfer of homologous or heterologous DNA into non-human organisms, like plants or other microorganisms. In accordance with a particular embodiment, the constructs are based on binary vectors (overview of a binary vector: Hellens et al., 2000). As a rule, they contain prokaryotic regulatory sequences, such as replication origin and selection markers, for the multiplication in microorganisms such as Escherichia coli and Agrobacterium tumefaciens. Vectors can further contain agrobacterial T-DNA sequences for the transfer of DNA into plant genomes or other eukaryotic regulatory sequences for transfer into other eukaryotic cells, e.g. Saccharomyces sp. or other prokaryotic regulatory sequences for the transfer into other prokaryotic cells, e.g. Corynebacterium sp. or Bacillus sp. For the transformation of plants, the right border sequence, which comprises approximately 25 base pairs, of the total agrobacterial T-DNA sequence is advantageously included. Usually, the plant transformation vector constructs according to the invention contain T-DNA sequences both from the right and from the left border region, which contain expedient recognition sites for site-specific acting enzymes, which, in turn, are encoded by some of the vir genes.

Alternatively the nuleic acids of the invention are cloned into vectors, which are designed for the direct transformation of organelles such as for example plastids. Generally such vectors additionally carry a specific resistance gene (as mentioned above), like the spectomycin resistance gene (aad) under control of a plastid regulatory sequence and two adjacent plastome sequences of the target organism, which mediated the directed insertion of the sequences of interest, e.g. the resistance gene and the expression cassette, into the plastidal genome through homologous recombination. As transformation can be achieved by particle bombardment or other physical or chemical methods e.g. PEG treatment or microinjection, the vectors do not need to contain the elements necessary for agrobacterial T-DNA transfer (see below).

In order to introduce, into a plant, the nucleic acid molecule of the invention or used in the process according to the invention, it has proved advantageous first to transfer them into an intermediate host, for example a bacterium or a eukaryotic unicellular cell. The transformation into E. coli, which can be carried out in a manner known per se, for example by means of heat shock or electroporation, has proved itself expedient in this context. Thus, the transformed E. coli colonies can be analyzed for their cloning efficiency. This can be carried out with the aid of a PCR. Here, not only the identity, but also the integrity, of the plasmid construct can be verified with the aid of a defined colony number by subjecting an aliquot of the colonies to said PCR. As a rule, universal primers which are derived from vector sequences are used for this purpose, it being possible, for example, for a forward primer to be arranged upstream of the start ATG and a reverse primer to be arranged downstream of the stop codon of the codogenic gene segment. The amplificates are separated by electrophoresis and assessed with regard to quantity and quality.

The nucleic acid constructs, which are optionally verified, are subsequently used for the transformation of the plants or other hosts, e.g. other eukaryotic cells or other prokaryotic cells. To this end, it may first be necessary to obtain the constructs from the intermediate host. For example, the constructs may be obtained as plasmids from bacterial hosts by a method similar to conventional plasmid isolation.

The nucleic acid molecule of the invention or used in the process according to the invention can also be introduced into modified viral vectors like baculovirus vectors for expression in insect cells or plant viral vectors like tobacco mosaic virus or potato virus X-based vectors. Approaches leading to the expression of proteins from the modified viral genome including the nucleic acid molecule of the invention or used in the process according to the invention involve for example the inoculation of tobacco plants with infectious RNA transcribed in vitro from a cDNA copy of the recombinant viral genome. Another approach utilizes the transfection of whole plants from wounds inoculated with Agrobacterium tumefaciens containing cDNA copies of recombinant plus-sense RNA viruses. Different vectors and virus are known to the skilled worker for expression in different target e.g. production plants.

A large number of methods for the transformation of plants are known. Since, in accordance with the invention, a stable integration of heterologous or additional homologous DNA into the genome of plants is advantageous, the T-DNA-mediated transformation has proved expedient in particular. For this purpose, it is first necessary to transform suitable vehicles, in particular agrobacteria, with a codogenic gene segment or the corresponding plasmid construct comprising the nucleic acid molecule of the invention or an expression cassette according to the invention. This can be carried out in a manner known per se. For example, said nucleic acid construct of the invention, like said expression cassette or said plasmid construct, which has been generated in accordance with what has been detailed above, can be transformed into competent agrobacteria by means of electroporation or heat shock. In principle, one must differentiate between the formation of cointegrated vectors on the one hand and the transformation with binary vectors on the other hand. In the case of the first alternative, the constructs, which comprise the codogenic gene segment or the nucleic acid molecule of the invention have no T-DNA sequences, but the formation of the cointegrated vectors or constructs takes place in the agrobacteria by homologous recombination of the construct with T-DNA. The T-DNA is present in the agrobacteria in the form of Ti or Ri plasmids in which exogenous DNA has expediently replaced the oncogenes. If binary vectors are used, they can be transferred to agrobacteria either by bacterial conjugation or by direct transfer. These agrobacteria expediently already comprise the vector bearing the vir genes (currently referred to as helper Ti (Ri) plasmid). As mentioned before the stable integration of the heterologous (or additional homologous) nucleic acids into the plastidial genome may also be advantageously.

One or more markers may expediently also be used together with the nucleic acid construct, like the expression cassette, or the vector of the invention and, if plants or plant cells shall be transformed together with the T-DNA, with the aid of which the isolation or selection of transformed organisms, such as agrobacteria or transformed plant cells, is possible. These marker genes enable the identification of a successful transfer of the nucleic acid molecules according to the invention via a series of different principles, for example via visual identification with the aid of fluorescence, luminescence or in the wavelength range of light which is discernible for the human eye, by a resistance to herbicides or antibiotics, via what are known as nutritive markers (auxotrophism markers) or antinutritive markers, via enzyme assays or via phytohormones. Examples of such markers which may be mentioned are GFP (=green fluorescent protein); the luciferin/luceferase system, the [3-galactosidase with its colored substrates, for example X-Gal, the herbicide resistances to, for example, imidazolinone, glyphosate, phosphinothricin or sulfonylurea, the antibiotic resistances to, for example, bleomycin, hygromycin, streptomycin, kanamycin, tetracyclin, chloramphenicol, ampicillin, gentamycin, geneticin (G418), spectinomycin or blasticidin, to mention only a few, nutritive markers such as the utilization of mannose or xylose, or antinutritive markers such as the resistance to 2-deoxyglucose. This list is a small number of possible markers. The skilled worker is very familiar with such markers. Different markers are preferred, depending on the organism and the selection method. In case of plastidal transformation methods other marker genes known to a person skilled in the art may be used, but also the ones mentioned above, preferably e.g. the spectomycin resistance gene (aadA).

As a rule, it is desired that the plant nucleic acid constructs, plant expression cassettes, are flanked by T-DNA at one or both sides of the codogenic gene segment. This is particularly useful when bacteria of the species Agrobacterium tumefaciens or Agrobacterium rhizogenes are used for the transformation. A method, which is preferred in accordance with the invention, is the transformation with the aid of Agrobacterium tumefaciens. However, biolistic methods may also be used advantageously for introducing the sequences in the process according to the invention, and the introduction by means of PEG is also possible. The transformed agrobacteria can be grown in the manner known per se and are thus available for the expedient transformation of the plants. The plants or plant parts to be transformed are grown or provided in the customary manner. The transformed agrobacteria are subsequently allowed to act on the plants or plant parts until a sufficient transformation rate is reached. Allowing the agrobacteria to act on the plants or plant parts can take different forms. For example, a culture of morphogenic plant cells or tissue may be used. After the T-DNA transfer, the bacteria are, as a rule, eliminated by antibiotics, and the regeneration of plant tissue is induced. This is done in particular using suitable plant hormones in order to initially induce callus formation and then to promote shoot development.

Plant cells, plant tissues etc. may be transformed transient or stable. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least the flower primordia. The plant is subsequently grown on until the seeds of the treated plant are obtained (Clough and Bent, Plant J. 16, 735 (1998)). To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Further advantageous transformation methods, in particular for plants, are known to the skilled worker and are described herein.

Further advantageous and suitable methods are protoplast transformation by poly(ethylene glycol)-induced DNA uptake, the “biolistic” method using the gene cannon—referred to as the particle bombardment method, electroporation, the incubation of dry embryos in DNA solution, microinjection and gene transfer mediated by Agrobacterium. Said methods are described by way of example in Jenes B. et al., Techniques for Gene Transfer, in: Transgenic Plants, Vol. 1, Engineering and Utilization, eds. Kung S. D. and Wu R., Academic Press (1993) 128-143 and in Potrykus, Annu. Rev. Plant Physiol. Plant Molec. Biol. 42, 205 (1991)). The nucleic acids or the construct, e.g. the expression cassette, to be expressed is preferably cloned into a vector, which is suitable for transforming Agrobacterium tumefaciens, for example pBin19 (Bevan et al., Nucl. Acids Res. 12, 8711 (1984)). Agrobacteria transformed by an expression vector according to the invention may likewise be used in known manner for the transformation of plants such as test plants like Arabidopsis or crop plants such as cereal crops, corn, oats, rye, barley, wheat, soybean, rice, cotton, sugar beet, canola, sunflower, flax, hemp, potatoes, tobacco, tomatoes, carrots, paprika, oilseed rape, tapioca, cassava, arrowroot, tagetes, alfalfa, lettuce and the various tree, nut and vine species, in particular oil-containing crop plants such as soybean, peanut, castor oil plant, sunflower, corn, cotton, flax, oilseed rape, coconut, oil palm, safflower (Carthamus tinctorius) or cocoa bean, or in particular corn, wheat, soybean, rice, cotton and canola, e.g. by bathing bruised leaves or chopped leaves in an agrobacterial solution and then culturing them in suitable media. The transformation of plants by means of Agrobacterium tumefaciens is described, for example, by Hofgen and Willmitzer in Nucl. Acid Res. 16, 9877 (1988) or is known inter alia from White F. F., “Vectors for Gene Transfer in

Higher Plants”; in Transgenic Plants, Vol. 1, Engineering and Utilization, eds. Kung S. D. and Wu R., Academic Press, 1993, pp. 15-38. For the transformation of plastids physical methods like PEG-treatment (O'Neil et al., Plant Journal. 3, 729 (1993), Golds et al., BioTechnology 11, 95 (1993)), microinjection (Knoblauch et al., Nat. Biotech. 17, 906 (1999)) or biolistics (Svab et al., Proc. Natl. Acad. Sci. USA 90, 8526 (1990)) are preferred. Such transformation methods are especially useful for the direct transformation of plastids and are well known to the skilled worker.

The expression of the nucleic acid molecules used in the process according the present invention may be desired alone or in combination with other genes or nucleic acid molecules. Multiple nucleic acid molecules conferring the expression of advantageous genes can be introduced via the simultaneous transformation of several individual suitable nucleic acid constructs, i.e. expression constructs, or, preferably, by combining several expression cassettes on one construct. It is also possible to transform the recipient non-human organisms stepwise with several vectors which in each case comprises a single expression cassette or several expression cassettes.

In addition to the sequence mentioned in Table I, application no. 7, column 5 or 8, preferably the coding region thereof, or its homologs or fragments, it may be advantageous additionally to express and/or mutate further genes in the non-human organisms. Especially advantageously, additionally at least one further gene of the biosynthetic pathway of the fine chemical is expressed in the non-human organisms such as plants or microorganisms. It is also possible that the regulation of the natural genes has been modified advantageously so that the gene and/or its gene product is no longer subject to the regulatory mechanisms which exist in the non-human organisms. This leads to an increased synthesis of the respective fine chemical vitamin E or its precursor 2,3-dimethyl-5-phytylquinol resp., in particular, of alpha- and/or gamma-tocopherol, resp. since, for example, feedback regulations no longer exist to the same extent or not at all. In addition it might be advantageously to combine the nucleic acids sequences of the invention containing the sequences shown in the respective line in Table I, application no. 7, column 5 or 8, preferably the coding region thereof, or homologs or fragments thereof, with genes which generally support or enhance the growth or yield of the target non-human organisms, for example genes which lead to faster growth rate of non human organism like microorganisms or plants or genes which produce stress-, pathogen-, or herbicide-resistant plants.

In a further embodiment of the process of the invention, non-human organisms are grown, in which there is in addition to the expression of the nucleic acid molecule according to the invention simultaneous expression of at least one nucleic acid molecule or one of the genes which code for proteins involved in the vitamin metabolism, in particular in the vitamin E or its precursor 2,3-dimethyl-5-phytylquinol resp., in particular, of alpha-, beta-, and/or gamma-tocopherol synthesi , such as the homogentisate phytyltransferase (HPT) or the enzymes catalysing the subsequent cyclization and methylation reactions, γ-tocopherol methyl transferase (γTMT), prenyltransferases that condense prenyl groups with allylic chains and those that condense prenyl chains with aromatic groups and others. The expression of these genes can lead to an increased synthesis of the essential vitamin E or its precursor 2,3-dimethyl-5-phytylquinol resp., in particular, of alpha- and/or gamma-tocopherol, resp.

In a further advantageous embodiment of the process of the invention, the non-human organisms used in the process are those in which simultaneously in addition a fine chemical degrading protein is attenuated, in particular by reducing the rate of expression of the corresponding gene.

In another embodiment of the process of the invention, the non-human organisms used in the process are those in which simultaneously in addition at least one of the aforementioned nucleic acids or of the aforementioned genes is mutated in such a way that the enzymatic or biological activity of the corresponding fine chemical degrading protein is partially reduced or completely blocked. A reduction in the enzymatic or biological activity means an enzymatic or biological activity, which is reduced by at least 10%, advantageously at least 20%, 30%, 40%, 50%, 60%, 70% or more, compared with the starting organism.

If it is intended to transform the host cell, in particular the plant cell, with several constructs, expression cassettes or vectors, the marker of a preceding transformation must be removed or a further marker employed in a following transformation. The markers can be removed from the host cell, in particular the plant cell, as described herein below via methods with which the skilled worker is familiar. These methods may be not used for multiple transformed host cell only but also for single transformed host cells. In particular plants without a marker, in particular without resistance to antibiotics, are an especially preferred embodiment of the present invention.

In the process according to the invention, the nucleic acid molecules used in the process according to the invention are advantageously linked operably to one or more regulatory signals in order to increase gene expression. These regulatory sequences are intended to enable the specific expression of the genes and the expression of protein. Depending on the host organism for example plant or microorganism, this may mean, for example, that the gene is expressed and/or overexpressed after induction only, or that it is expressed and/or overexpressed constitutively. These regulatory sequences are, for example, sequences to which the inductors or repressors bind and which thus regulate the expression of the nucleic acid. In addition to these novel regulatory sequences, or instead of these sequences, the natural regulation of these sequences may still be present before the actual structural genes and, if appropriate, may have been genetically modified so that the natural regulation has been switched off and gene expression has been increased. However, the nucleic acid construct of the invention suitable as expression cassette (=expression construct =gene construct) can also be simpler in construction, that is to say no additional regulatory signals have been inserted before the nucleic acid molecule or the homolog or fragment thereof, and the natural promoter together with its regulation has not been removed. Instead, the natural regulatory sequence has been mutated in such a way that regulation no longer takes place and/or gene expression is increased.

These modified promoters can also be introduced on their own before the natural gene in the form of part sequences (=promoter with parts of the nucleic acid sequences according to the invention) in order to increase the activity. Moreover, the gene construct can advantageously also comprise one or more of what are known as enhancer sequences in operable linkage with the promoter, and these enable an increased expression of the nucleic acid sequence. Also, it is possible to insert additional advantageous sequences at the 3′ end of the DNA sequences, such as, for example, further regulatory elements or terminators. In another preferred embodiment, the natural or created expression cassette is further modified in such a manner, that a nucleic acid sequence encoding a transit peptide is functionally introduced between the regulatory and the coding region such, that a functionally preprotein is expressed, which is targeted to the organelles such as plastids or mitochondria, preferably the plastids.

The nucleic acid molecules, which encode proteins according to the invention and nucleic acid molecules, which encode other polypeptides may be present in one nucleic acid construct, expression cassette or vector or in respective several ones. Advantageously, only one copy of the nucleic acid molecule of the invention or its encoding genes is present in the nucleic acid construct, expression cassette or vector. Several vectors, nucleic acid construct, or expression cassettes can be expressed together in the host organism. The nucleic acid molecule, the nucleic acid construct or the expression cassette according to the invention can be inserted in a vector and be present in the cell in a free form. If a stable transformation is preferred, a vector is used, which is stably duplicated over several generations or which is else inserted into the genome. In the case of plants, integration into the genome of e.g. plastids or mitochondria or, in particular, into the nuclear genome may have taken place. For the insertion of more than one gene in the host genome the genes to be expressed are present together in one gene construct, for example in above-described vectors bearing a plurality of genes.

As a rule, regulatory sequences for the expression rate of a gene are located upstream (5′), within, and/or downstream (3′) relative to the coding sequence of the nucleic acid molecule of the invention or another codogenic gene segment. They control in particular transcription and/or translation and/or the transcript stability. The expression level is dependent on the conjunction of further cellular regulatory systems, such as the protein biosynthesis and degradation systems of the cell.

Regulatory sequences include transcription and translation regulating sequences or signals, e.g. sequences located upstream (5′), which concern in particular the regulation of transcription or translation initiation, such as promoters or start codons, and sequences located downstream (3′), which concern in particular the regulation of transcription or translation termination and transcript stability, such as polyadenylation signals or stop codons. Regulatory sequences can also be present in transcribed coding regions as well in transcribed non-coding regions, e.g. in introns, as for example splicing sites, promoters for the regulation of expression of the nucleic acid molecule according to the invention in a cell and which can be employed are, in principle, all those which are capable of stimulating the transcription of genes in the organisms in question, such as microorganisms or plants. Suitable promoters, which are functional in these non-human organisms are generally known. They may take the form of constitutive or inducible promoters. Suitable promoters can enable the development- and/or tissue-specific expression in multi-cell eukaryotes; thus, leaf-, root-, flower-, seed-, stomata-, tuber-, fruit- or pollen-specific promoters may advantageously be used in plants. Furthermore in case of direct transformation of organelles such as plastids, promoters recognized by the plastid RNA-polymerases such as the plastid encoded Escherichia coli-like RNA polymerase or the nuclear encoded plastid RNA polymerase may advantageously be used.

The regulatory sequences or factors can, as described above, have a positive effect on, the expression of the genes introduced, thus increasing their expression. Thus, an enhancement of the expression can advantageously take place at the transcriptional level by using strong transcription signals such as strong promoters and/or strong enhancers. In addition, enhancement of expression on the translational level is also possible, for example by introducing translation enhancer sequences, e.g., the omega-enhancer e.g. improving the ribosomal binding to the transcript, or by increasing the stability of the mRNA, e.g. by replacing the 3′UTR coding region by a region encoding a 3′UTR known as conferring an high stability of the transcript or by stabilization of the transcript through the elimination of transcript instability, so that the mRNA molecule is translated more often than the wild type. For example in plants AU-rich elements (AREs) and DST (downstream) elements destabilized transcripts. Mutagenesis studies have demonstrated that residues within two of the conserved domains, the ATAGAT and the GTA regions, are necessary for instability function. Therefore removal or mutation of such elements would obviously lead to more stable transcripts, higher transcript rates and higher protein activity. Translation enhancers are also the “overdrive sequence”, which comprises the tobacco mosaic virus 5′-untranslated leader sequence and which increases the protein/RNA ratio (Gallie et al., Nucl. Acids Research 15, 8693 (1987)).

Enhancers are generally defined as cis active elements, which can stimulate gene transcription independent of position and orientation. Different enhancers have been identified in plants, which can either stimulate transcription constitutively or tissue or stimuli specific.

Well known examples for constitutive enhancers are the enhancer from the 35S promoter (Odell et al., Nature 313, 810 (1985)) or the ocs enhancer (Fromm et al., Plant Cell 1, 977 (1989)). Other examples are the G-Box motif tetramer which confers high-level constitutive expression in dicotyledonous and monocotyledonous plants (Ishige et al., Plant Journal 18, 443 (1999)) or the petE, a NT-rich sequence which act as quantitative enhancers of gene expression in transgenic tobacco and potato plants (Sandhu et al., Plant Mol Biol. 37 (5), 885 (1998)). Beside that, a large variety of cis-active elements have been described which contribute to specific expression pattern, like organ specific expression or induced expression in response to biotic or abiotic stress. Examples are elements, which provide pathogen or wound-induced expression (Rushton, Plant Cell 14, 749 (2002)) or guard cell-specific expression (Plesch, Plant Journal 28, 455 (2001)).

Advantageous regulatory sequences for the expression of the nucleic acid molecule according to the invention in microorganisms are present for example in promoters such as the cos, tac, rha, trp, tet, trp-tet, Ipp, lac, !pp-lac, laclq-, T7, T5, T3, gal, trc, ara, SP6, Λ-PR or Λ-PL promoter, which are advantageously used in Gram-negative bacteria. Further advantageous regulatory sequences are present for example in the Gram-positive promoters amy, dnaK, xylS and SPO2, in the yeast or fungal promoters ADC1, MFα, AC, P-60, UASH, MCB, PHO, CYC1, GAPDH, TEF, rp28, ADH. Promoters, which are particularly advantageous, are constitutive, tissue or compartment specific or inducible promoters. In general, “promoter” is understood as meaning, in the present context, a regulatory sequence in a nucleic acid molecule, which mediates the expression of a coding sequence segment of a nucleic acid molecule. In general, the promoter is located upstream to the coding sequence segment. Some elements, for example expression-enhancing elements such as enhancer may, however, also be located downstream or even in the transcribed region.

In principle it is possible to use all natural promoters with their regulation sequences like those named above for the expression cassette according to the invention and the method according to the invention. Also, synthetic promoters may advantageously be used, either additionally or alone, for example synthetic promoters mediating seed-specific expression such as described in, for example, WO 99/16890. In the preparation of an expression cassette various DNA fragments can be manipulated in order to obtain a nucleotide sequence, which usefully reads in the correct direction and is equipped with a correct reading frame. To connect the DNA fragments (=nucleic acids according to the invention) to one another adaptors or linkers may be attached to the fragments. The promoter and the terminator regions can usefully be provided in the transcription direction with a linker or polylinker containing one or more restriction points for the insertion of this sequence. Generally, the linker has 1 to 10, mostly 1 to 8, preferably 2 to 6, restriction points. In general the size of the linker inside the regulatory region is less than 100 bp, frequently less than 60 bp, but at least 5 bp. The promoter may be both native or homologous as well as foreign or heterologous to the host non-human organism, for example to the host plant. In the 5′-3′ transcription direction the expression cassette contains the promoter, a nucleic acid molecule which is shown in the respective line in Table I, column 5 or 8, preferably the coding region, a homolog or a fragment thereof, and a region for transcription termination. Different termination regions can be exchanged for one another in any desired fashion.

As described above, the transcription of the genes introduced should advantageously be terminated by suitable terminators at the 3′ end of the genes introduced (behind the stop codon). A terminator, which may be used for this purpose is, for example, the OCS1 terminator, the nos3 terminator or the 35S terminator. As is the case with the promoters, different terminator sequences should be used for each gene in case several genes are introduced. Terminators, which are useful in microorganisms are for example the fimA terminator, txn terminator or trp terminator. Such terminators can be rho-dependent or rho-independent.

In an embodiment of the present invention the nucleic acid construct of the present invention comprises a nucleic acid molecule as depicted in the respective line in Table I, application no. 7, column 5 or 8, preferably the coding region thereof, a fragment or a homolog thereof, and a plant promoter, such as the Big 35S promoter, the DC3 promoter, the PC Ubi promoter, the LegB4-promoter, the Super promoter, the USP promoter or the like. In another embodiment the nucleic acid construct of the present invention comprises a nucleic acid molecule as depicted in the respective line in Table I, application no. 7, column 5 or 8, preferably the coding region thereof, a fragment or a homolog thereof, and a plant promoter such as, for example, the USP, the LegB4-, the DC3 promoter or the ubiquitin promoter from parsley or other herein mentioned promoters. In a further embodiment the nucleic acid construct of the present invention comprises a nucleic acid molecule as depicted in the respective line in Table I, application no. 7, column 5 or 8, preferably the coding region thereof, a fragment or a homolog thereof, and a plant promoter, such as the Big 35S promoter, the PC Ubi promoter, the Super promoter, the USP promoter or the like. In addition different terminators may advantageously be used in these nucleic acid constructs; examples for transcriptional termination are polyadenylation signals.

In order to ensure the stable integration, into the transgenic plant, of nucleic acid molecules used in the process according to the invention in combination with further genes, especially biosynthesis genes, over a plurality of generations, it may be advantageous to express each of the respective coding regions used in the process under the control of its own, preferably unique, promoter.

The nucleic acid construct is advantageously constructed in such a way that a promoter is followed by a suitable cleavage site for insertion of the nucleic acid to be expressed, advantageously in a polylinker, followed, if appropriate, by a terminator located behind the polylinker. If appropriate, this order is repeated several times so that several genes are combined in one construct and thus can be introduced into the transgenic plant in order to be expressed. The sequence is advantageously repeated up to three times. For the expression, the nucleic acid sequences are inserted via the above-mentioned suitable cleavage site, for example in the polylinker behind the promoter. It is advantageous for each nucleic acid sequence to have its own promoter and, if appropriate, its own terminator, as mentioned above. However, it is also possible to insert several nucleic acid sequences behind a promoter and, if appropriate, before a terminator if a polycistronic transcription is possible in the host or target cells. In this context, the insertion site, or the sequence of the nucleic acid molecules inserted, in the nucleic acid construct is not decisive, that is to say a nucleic acid molecule can be inserted in the first or last position in the cassette without this having a substantial effect on the expression. However, it is also possible to use only one promoter type in the construct.

Accordingly, in a preferred embodiment, the nucleic acid construct according to the invention confers expression of the nucleic acid molecule of the invention, and, optionally further genes, in a plant and comprises one or more plant regulatory elements. Said nucleic acid construct according to the invention advantageously encompasses beneath the nucleic acid molecule of the invention a plant promoter or a plant terminator or a plant promoter and a plant terminator. In another embodiment said nucleic acid construct according to the invention advantageously encompasses beneath the nucleic acid molecule of the invention a microorganism promoter or a microorganism terminator or a microorganism promoter and a microorganism terminator.

A “plant” promoter comprises regulatory elements, which mediate the expression of a coding sequence segment in plant cells. The plant promoter can originate from a plant cell, e.g. from the plant, which is transformed with the nucleic acid construct, expression cassette, or vector as described herein. However, a plant promoter does not need to be of plant origin, but may originate from viruses or microorganisms, in particular for example from viruses which attack plant cells. This also applies to other plant regulatory signals, for example in plant terminators The term plant promoter shall also encompass organelle, especially plastidal promoters.

A nucleic acid construct suitable for plant expression preferably comprises regulatory elements which are capable of controlling the expression of genes in plant cells and which are operably linked so that each sequence can fulfill its function. Accordingly, the nucleic acid construct can also comprise transcription terminators. Examples for transcriptional termination are polyadenylation signals. Preferred polyadenylation signals are those which originate from Agrobacterium tumefaciens T-DNA, such as the gene 3 of the Ti plasmid pTiACH5, which is known as octopine synthase (Gielen et al., EMBO J. 3, 835 (1984)) or functional equivalents thereof, but all the other terminators which are functionally active in plants are also suitable.

Other preferred sequences for use in operable linkage in gene expression constructs are targeting sequences, which are required for targeting the gene product into specific cell compartments (for a review, see Kermode, Crit. Rev. Plant Sci. 15 (4), 285 (1996) and references cited therein), for example into the vacuole, the nucleus, all types of plastids, such as amyloplasts, chloroplasts, chromoplasts, the extracellular space, the mitochondria, the endoplasmic reticulum, elaioplasts, peroxisomes, glycosomes, and other compartments of cells or extracellular; preferred are sequences, which are involved in targeting to plastids as mentioned above. Sequences, which must be mentioned in this context are, in particular, the signal-peptide- or transit-peptide-encoding sequences which are known per se. For example, plastid-transit-peptide-encoding sequences enable the targeting of the expression product into the plastids of a plant cell. Targeting sequences are also known for eukaryotic and to a lower extent for prokaryotic organisms and can advantageously be operable linked with the nucleic acid molecule of the present invention as shown in Table I, application no. 7, columns 5 and 8, preferably the coding region thereof, or homologs or fragments thereof, and described herein to achieve an expression in one of said compartments or extracellular.

For expression in plants, the nucleic acid molecule must, as described above, be linked operably to or comprise a suitable promoter which expresses the gene at the right point in time and in a cell- or tissue-specific manner. Usable promoters are constitutive promoters (Benfey et al., EMBO J. 8, 2195 (1989)), such as those which originate from plant viruses, such as 35S CAMV (Franck et al., Cell 21, 285 (1980)), 19S CaMV (see also U.S. Pat. No. 5,352,605 and WO 84/02913), 34S FMV (Sanger et al., Plant. Mol. Biol., 14, 433 (1990)), the parsley ubiquitin promoter, or plant promoters such as the Rubisco small subunit promoter described in U.S. Pat. No. 4,962,028 or the plant promoters PRP1 (Ward et al., Plant. Mol. Biol. 22 (1993)), SSU, PGEL1, OCS (Leisner, Proc. Natl. Acad. Sci. USA 85 (5), 2553 (1988)), lib4, usp, mas (Comai, Plant Mol. Biol. 15 (3), 373 (1990)), STLS1, ScBV (Schenk, Plant Mol. Biol. 39 (6),1221 (1999)), B33, SAD1 or SAD2 (flax promoters, Jain et al., Crop Science, 39 (6), 1696 (1999)) or nos (Shaw et al., Nucleic Acids Res. 12 (20), 7831 (1984)). Stable, constitutive expression of the proteins according to the invention into a plant can be advantageous. However, inducible expression of the polypeptide of the invention may be advantageous, if, for example, a late expression before the harvest is of advantage.

The expression of plant genes can also be facilitated as described above via a chemical inducible promoter (for a review, see Gatz, Annu. Rev. Plant Physiol. Plant Mol. Biol. 48, 89 (1997)). Chemically inducible promoters are particularly suitable when it is desired to express the gene in a time-specific manner. Examples of such promoters are a salicylic acid inducible promoter (WO 95/19443), a benzenesulfonamide inducible promoter (EP 388 186), and abscisic acid-inducible promoter (EP 335 528), a tetracyclin-inducible promoter (Gatz et al., Plant J. 2, 397 (1992)), a cyclohexanol- or ethanol-inducible promoter (WO 93/21334) or others as described herein.

Other suitable promoters are those which react to biotic or abiotic stress conditions, for example the pathogen-induced PRP1 gene promoter (Ward et al., Plant. Mol. Biol. 22, 361 (1993)), the auxin-induced GH3-promotor (Liu et al., Plant Cell 6, 645 (1994)), the cold/dehydration/ABA-induced Cor15A-promoter (Baker et al., Plant Mol. Biol. 24, 701 (1994)), the cold/drought/salt/ABA/wound-induced Cor78 promoter (Ishitani et al., Plant Cell 9, 1935 (1997), Yamaguchi-Shinozaki and Shinozaki, Plant Cell 6, 251 (1994)), the cold/dehydrationinduced Rci2A-promoter (Capel et al., Plant Physiol. 115, 569 (1997)), the drought/salt-induced Rd22-promoter (Yamaguchi-Shinozaki and Shinozaki, Mol. Gen. Genet. 238, 17 (1993)), the salt-induced RD29A-promoter (Yamaguchi-Shinozalei et al., Mol. Gen. Genet. 236, 331 (1993)), the salt-induced ARSK1-root promoter (Hwang and Goodman, Plant J. 8, 37 (1995), the saltinduced PtxA-root promoter (GenBank accession X67427), the tomato heat-inducible hsp80 promoter (U.S. Pat. No. 5,187,267), the potato chill-inducible alpha-amylase promoter (WO 96/12814) or the wound-inducible pinII promoter (EP-A-0 375 091) or others as described herein.

Preferred promoters are in particular those which bring about gene expression in tissues and organs, such as leaves, roots, seeds, pollen or xylem. Examples of tissue preferred or organ preferred promoters include, but are not limited to fruit-preferred, ovule-preferred, male tissue-preferred, seed-preferred, pollen-preferred integument-preferred, tuber-preferred, stalk-preferred, pericarp-preferred, leaf-preferred, stigma-preferred, pollen-preferred, anther-preferred, petal-preferred, sepal-preferred, pedicel-preferred, silique-preferred, stem-preferred, root-preferred promoters, and the like. Especially those promoters are preferred which bring about gene expression in tissues and organs in which the biosynthesis of vitamin E or its precursor 2,3-dimethyl-5-phytylquinol resp., in particular, of alpha- and/or gamma-tocopherol takes place, like in seed cells, such as endosperm cells and cells of the developing embryo. Seed promoters are preferentially expressed during seed development and/or germination. For example, seed preferred promoters can be embryo-preferred, endosperm preferred and seed coat-preferred (see Thompson et al., BioEssays 10, 108 (1989)). Examples of seed preferred promoters include, but are not limited to, cellulose synthase (celA), Cim1, gammazein, globulin-1, maize 19 kD zein (cZ19B1), and the like. Other suitable promoters are the oilseed rape napin gene promoter (U.S. Pat. No. 5,608,152), the Vicia faba USP promoter (Baeumlein et al., Mol Gen Genet, 225 (3), 459 (1991)), the Arabidopsis oleosin promoter (WO 98/45461), the Phaseolus vulgaris phaseolin promoter (U.S. Pat. No. 5,504,200), the Brassica Bce4 promoter (WO 91/13980), the bean arcs promoter, the carrot DcG3 promoter, or the Legumin B4 promoter (LeB4) (Baeumlein et al., Plant Journal, 2 (2), 233 (1992)), and promoters which bring about the seed-specific expression in monocotyledonous plants such as maize, barley, wheat, rye, rice and the like. Advantageous seed-specific promoters are the sucrose binding protein promoter (WO 00/26388), the phaseolin promoter and the napin promoter. Suitable promoters which must be considered are the barley Ipt2 or Ipt1 gene promoter (WO 95/15389 and WO 95/23230), and the promoters described in WO 99/16890 (promoters from the barley hordein gene, the rice glutelin gene, the rice oryzin gene, the rice prolamin gene, the wheat gliadin gene, the wheat glutelin gene, the maize zein gene, the oat glutelin gene, the sorghum kasirin gene and the rye secalin gene). Further suitable promoters are Amy32b, Amy 6-6 and Aleurain (U.S. Pat. No. 5,677,474), Bce4 (oilseed rape) (U.S. Pat. No. 5,530,149), glycinin (soya) (EP 571 741], phosphoenolpyruvate carboxylase (soya) (JP 06/62870), ADR12-2 (soya) (WO 98/08962), isocitrate lyase (oilseed rape) (U.S. Pat. No. 5,689,040]) or α-amylase (barley) (EP 781 8499. Other promoters which are available for the expression of genes in plants are leaf-specific promoters such as those described in DE-A 19 644 478 or light-regulated promoters such as, for example, the pea petE promoter.

Further suitable plant promoters are the cytosolic FBPase promoter or the potato ST-LSI promoter (Stockhaus et al., EMBO J. 8, 2445 (1989)), the Glycine max phosphoribosylpyrophosphate amidotransferase promoter (GenBank Accession No. U87999) or the noduline-specific promoter described in EP 249 676.

Promoters, which are particularly suitable, are those which bring about plastidspecific expression. Suitable promoters such as the viral RNA polymerase promoter are described in WO 95/16783 and WO 97/06250, and the Arabidopsis clpP promoter, which is described in WO 99/46394.

Promoters, which are used for the strong expression of heterologous sequences as well as additional homologous sequences in as many tissues as possible, in particular also in leaves, are, in addition to several of the above-mentioned viral and bacterial promoters, preferably, plant promoters of actin or ubiquitin genes such as, for example, the rice actin1 promoter. Further examples of constitutive plant promoters are the sugarbeet V-ATPase promoters (WO 01/14572). Examples of synthetic constitutive promoters are the Super promoter (WO 95/14098) and promoters derived from G-boxes (WO 94/12015). If appropriate, chemical inducible promoters may furthermore also be used, as described in EP 388 186, EP 335 528, WO 97/06268.

Promoters, which are particularly suitable, are e.g. the super-promoter (Ni et al., Plant Journal 7, 661 (1995)), the ubiquitin promoter (Callis et al., J. Biol. Chem., 265, 12486 (1990); U.S. Pat. No. 5,510,474; U.S. Pat. No. 6,020,190; Kawalleck et al., Plant. Molecular Biology, 21, 673 (1993)) or the 34S promoter (GenBank Accession numbers M59930 and X16673). Promoters useful in the expression cassettes of the invention include, but are not limited to, the major chlorophyll a/b binding protein promoter, histone promoters, the Ap3 promoter, the [3-conglycin promoter, the napin promoter, the soybean lectin promoter, the maize 15kD zein promoter, the 22kD zein promoter, the 27kD zein promoter, the g-zein promoter, the waxy, shrunken 1, shrunken 2 and bronze promoters, the Zm13 promoter (U.S. Pat. No. 5,086,169), the maize polygalacturonase promoters (PG) (U.S. Pat. Nos. 5,412,085 and 5,545,546), and the SGB6 promoter (U.S. Pat. No. 5,470,359), the rice cyclophilin promoter (Buchholz et al., Plant Mol. Biol.25 (5), 837 (1994)), the glutelin (rice) promoter (Takaiwa et al., Mol. Gen. Genet. 208, 15 (1986), Takaiwa et al., FEBS letts. 221, 43 (1987), the rice prolamin NRP33 promoter and the rice aglobulin Glb-1 promoter (Wu et al., Plant cell Physiology 39 (8), 885 (1998),as well as synthetic or other natural promoters.

As already mentioned herein, further regulatory sequences, which may be expedient, if appropriate, also include sequences, which target the transport and/or the localization of the expression products. Sequences, which must be mentioned in this context are, in particular, the signal-peptide- or transit-peptide-encoding sequences which are known per se. For example, plastid-transit-peptide-encoding sequences enable the targeting of the expression product into the plastids of a plant cell.

One embodiment of the present invention also relates to a method for generating a vector, which comprises the insertion of the nucleic acid molecule according to the invention or the expression cassette according to the invention into a nucleic acid molecule to give the vector. The vector can, for example, be introduced in to a cell, e.g. a microorganism or a plant cell, a plant or a part therof, as described herein for the nucleic acid construct, or below under transformation or transfection or shown in the examples. A transient or stable transformation of the host or target cell is possible, however, a stable transformation is preferred. The vector according to the invention is preferably a vector, which is suitable for expressing the polypeptide according to the invention in a microorganism or a plant cell, a plant or a part thereof.

The method can thus also encompass one or more steps for integrating regulatory signals into the vector, in particular signals, which mediate the expression in microorganisms or plant cells, plants or respective parts thereof.

Accordingly, the present invention also relates to a vector comprising the nucleic acid molecule characterized herein as part of a nucleic acid construct suitable for expression in plants and/or microorganisms or the nucleic acid molecule according to the invention.

The advantageous vectors of the invention comprise the nucleic acid molecules which encode proteins according to the invention, nucleic acid molecules which are used in the process, or nucleic acid construct suitable for plant expression or for microorganism expression comprising the nucleic acid molecules used, or expression cassettes according to the invention, either alone or in combination with further genes such as the biosynthesis or regulatory genes of the fine chemical metabolism e.g. with the genes mentioned herein above.

The recombinant expression vectors which are advantageously used in the process comprise the nucleic acid molecules according to the invention or the nucleic acid construct according to the invention, or the expression cassettes according to the invention, in a form which is suitable for expressing, in a host cell, the nucleic acid molecules according to the invention or described herein. Accordingly, the recombinant expression vectors in addition may comprise one or more regulatory signals selected on the basis of the host cells to be used for the expression, in operable linkage with the nucleic acid sequence to be expressed. Furthermore, if desired, in addition the vector can comprise plastome sequences of the recipient organism to facilitate integration into the plastidal genome by homologous recombination as mentioned above.

The recombinant expression vectors used can be designed specifically for the expression, in prokaryotic and/or eukaryotic cells, of nucleic acid molecules used in the process. This is advantageous since intermediate steps of the vector construction are frequently carried out in microorganisms for the sake of simplicity. For example, the genes according to the invention and other genes can be expressed in bacterial cells, insect cells (using baculovirus expression vectors), yeast cells and other fungal cells (Romanos, Yeast 8, 423 (1992); van den Hondel, in “More Gene Manipulations in Fungi”, ed. Bennet J. W., Lasure L. L., pp. 396-428, Academic Press, San Diego (1991); van den Hondel C. A. M. J. J., in “Applied Molecular Genetics of Fungi”, ed. Peberdy J. F. et al., pp. 1-28, Cambridge University Press, Cambridge(1991)), algae (Falciatore et al., Marine Biotechnology.1 (3), 239 (1999)) using vectors and following a transformation method as described in WO 98/01572, and preferably in cells of multi-celled plants (see Schmidt R. and Willmitzer L., Plant Cell Rep. 7, 583 (1988); “Plant Molecular Biology and Biotechnology”, C Press, Boca Raton, Fla., chapter 6/7, pp.71-119 (1993); White F. F., in “Transgenic Plants”, Bd. 1, Engineering and Utilization, ed.Kung and Wu R., Academic Press, 128-43 (1993); Potrykus, Annu. Rev. Plant Physiol. Plant Molec. Biol. 42, 205 (1991) (and references cited therein)). Suitable host cells are furthermore discussed in Goeddel, “Gene Expression Technology: Methods in Enzymology 185”, Academic Press, San Diego, Calif. (1990). As an alternative, the sequence of the recombinant expression vector can be transcribed and translated in vitro, for example using T7 promoter-regulatory sequences and T7 polymerase.

In the event it is necessary proteins can be expressed in prokaryotes using vectors comprising constitutive or inducible promoters, which control the expression of fusion proteins or nonfusion proteins as mentioned above.

Other vectors which are suitable in prokaryotic organisms are known to the skilled worker; these vectors are for example in E. coli pLG338, pACYC184, the pBR series, such as pBR322, the pUC series such as pUC18 or pUC19, the M113mp series, pKC30, pRep4, pHS1, pHS2, pPLc236, pMBL24, pLG200, pUR290, pIN-111113-B1, lambda-gt11 or pBdCI, in Streptomyces pIJ101, pIJ364, pIJ702 or pIJ361, in Bacillus pUB110, pC194 or pBD214, in Corynebacterium pSA77 or pAJ667.

In a further embodiment, the expression vector is a yeast expression vector. Examples of vectors for expression in the yeasts S. cerevisiae encompass pYeDesaturasecl (Baldari et al., Embo J. 6, 229 (1987)), pMFa (Kurjan and Herskowitz, Cell 30, 933 (1982)), pJRY88 (Schultz et al., Gene 54, 113(1987)) and pYES2 (Invitrogen Corporation, San Diego, Calif.). Vectors and methods for the construction of vectors which are suitable for use in other fungi, such as the filamentous fungi, encompass those which are described in detail in: van den Hondel C. A. M. J. J. in “Applied Molecular Genetics of Fungi”, Peberdy J. F., ed., pp. 1-28, Cambridge University Press, Cambridge (1991); or van den Hondel C. A. M. J. J. in “More Gene Manipulations in Fungi”, Bennet J. W. & Lasure L. L., ed., pp. 396-428, Academic Press, San Diego (1991). Examples of other suitable yeast vectors are 2alphaM, pAG-1, YEp6, YEp13 or pEM-BLYe23.

Further vectors, which may be mentioned by way of example, are pALS1, pIL2 or pBB116 in fungi or pLGV23, pGHlac+, pBIN19, pAK2004 or pDH51 in plants.

As an alternative, the nucleic acid sequences can be expressed in insect cells using baculovirus expression vectors. Baculovirus vectors, which are available for expressing proteins in cultured insect cells (for example Sf9 cells) encompass the pAc series (Smith et al., Mol. Cell Biol. 3, 2156 (1983)) and the pVL series (Lucklow and Summers, Virology 170, 31 (1989)).

The above-mentioned vectors are only a small overview of potentially suitable vectors. Further plasmids are known to the skilled worker and are described, for example, in “Cloning Vectors” (ed. Pouwels P. H., et al., Elsevier, Amsterdam-New York-Oxford, 1985, ISBN 0 444 904018). Further suitable expression systems for prokaryotic and eukaryotic cells, see the chapters 16 and 17 by Sambrook J., Fritsch E. F. and Maniatis T., “Molecular Cloning: A Laboratory Manual”, 2nd edition, Cold Spring Harbor Laboratory, Cold Spring Harbor Laboratory Press, Cold Spring Harbor, N.Y., 1989.

An embodiment of the invention relates to a vector where the nucleic acid molecule according to the invention is linked operably to regulatory sequences which permit the expression in a prokaryotic or eukaryotic host. A further embodiment of the invention relates to a vector in which a nucleic acid sequence encoding one of the polypeptides shown in the respective line in Table II, application no. 7, columns 5 or 8, or homologs or fragments thereof, is functionally linked to a targeting sequence such as a plastidial or mitochondrial targeting sequence. A further embodiment of the invention relates to a vector in which a nucleic acid sequence encoding one of the polypeptides shown in the respective line in Table II, application no. 7, columns 5 or 8, or homologs or fragments thereof, is functionally linked to (a) regulatory sequence which permit the expression in an organelle, such as plastids or mitochondria, preferably plastids.

A further embodiment of the invention relates to a vector which comprises a nucleic acid sequence encoding one of the polypeptides shown in the respective line in Table II, application no. 7, columns 5 or 8, or homologs or fragments thereof, but no additional targeting sequence.

Accordingly, one embodiment of the invention relates to a host cell, which has been transformed stably or transiently with the vector according to the invention or the nucleic acid molecule according to the invention or the nucleic acid construct according to the invention or the expression construct according to the invention.

The genetically modified cells may be regenerated to the respective non-human organism, for example a plant cell to a plant, by all of the methods known to those skilled in the art. Appropriate methods can be found in the publications referred to above by Kung S. D. and Wu R., Potrykus or Höfgen and Willmitzer.

Accordingly, a further aspect of the invention relates to transgenic non-human organisms transformed by at least one nucleic acid molecule, expression cassette or vector according to the invention as well as cells, cell cultures, tissue, parts—such as, for example, leaves, roots, pollen, etc. in the case of plant organisms—or reproductive material derived from such non-human organisms. The terms “host organism”, “host cell”, “recombinant (host) non-human organism” and “transgenic (host) cell” are used here interchangeably. Of course these terms relate not only to the particular host non-human organism or the particular target cell but also to the descendants or potential descendants of these non-human organisms or cells. Since, due to mutation or environmental effects certain modifications may arise in successive generations, these descendants need not necessarily be identical with the parental cell but nevertheless are still encompassed by the term as used here.

For the purposes of the invention “transgenic” or “recombinant” means with regard for example to a nucleic acid molecule, a nucleic acid construct, an expression cassette or a vector containing the nucleic acid molecule according to the invention or an non-human organism transformed with/by the nucleic acid molecule, nucleic acid construct, expression cassette or vector according to the invention all those constructions produced by genetic engineering methods in which either
(a) the nucleic acid molecule depicted in the respective line in Table I, column 5 or 8, preferably the coding region thereof, or homologs or fragments thereof; or
(b) a genetic control sequence functionally linked to the nucleic acid molecule described under (a), for example a 3′- and/or 5′-genetic control sequence such as a promoter or terminator, or
(c)
(a) and (b);
   are not found in their natural, genetic environment or have been modified by genetic engineering methods, wherein the modification may by way of example be a substitution, addition, deletion, inversion or insertion of one or more nucleotide residues. Natural genetic environment means the natural genomic or chromosomal locus in the organism of origin or inside the host non-human organism or presence in a genomic library. In the case of a genomic library the natural genetic environment of the nucleic acid sequence is preferably retained at least in part. The environment sequence borders the nucleic acid sequence at least on one side and has a sequence length of at least 50 bp, preferably at least 500 bp, particularly preferably at least 1,000 bp, most particularly preferably at least 5,000 bp. A naturally occurring expression cassette—for example the naturally occurring combination of the natural promoter of the nucleic acid sequence according to the invention with the corresponding gene—turns into a transgenic expression cassette when the latter is modified by unnatural, synthetic (“artificial”) methods such as by way of example a mutagenesis. In case a non-human organism is transformed by a homologous nucleic acid molecule or by a homologous expression cassette the resulting non-human organism represents also a respective transgenic non-human organism since the additional homologous nucleic acid molecule or the additional homologous expression cassette is not located in its original environment. Appropriate methods are described by way of example in U.S. Pat. No. 5,565,350 or WO 00/15815. In another embodiment the non-human organism comprises a heterologous nucleic acid molecule.

Suitable organisms or host organisms for the nucleic acid molecule, nucleic acid construct, expression cassette or vector according to the invention are advantageously in principle all non-human organisms, which are suitable for the expression of recombinant genes as described above. Preferred non-human organism are microorganism or plants as described above, in particular those plants, which can be transformed in a suitable manner. These include monocotyledonous and dicotyledonous plants. In an embodiment of the present invention plants like Arabidopsis, Asteraceae such as Calendula or crop plants such as soybean, peanut, castor oil plant, sunflower, flax, corn, cotton, oilseed rape, coconut, oil palm, safflower (Carthamus tinctorius) or cocoa bean are preferred. In another embodiment agriculturally useful plants such as cereals and grasses, for example Triticum spp., Zea mays, Hordeum vulgare, oats, Secale cereale, Oryza sativa, Pennisetum glaucum, Sorghum bicolor, Triticale, Agrostis spp., Cenchrus ciliaris, Dactylis glomerata, Festuca arundinacea, Lolium spp., Medicago spp. and Saccharum spp., legumes and oil crops, for example Brassica juncea, Brassica napus, Glycine max, Arachis hypogaea, Gossypium hirsutum, Cicer arietinum, Helianthus annuus, Lens culinaris, Linum usitatissimum, Sinapis alba, Trifolium repens and Vicia narbonensis, vegetables and fruits, for example bananas, grapes, Lycopersicon esculentum, asparagus, cabbage, watermelons, kiwi fruit, Solanum tuberosum, Beta vulgaris, cassava and chicory, trees, for example Coffea species, Citrus spp., Eucalyptus spp., Picea spp., Pinus spp. and Populus spp., medicinal plants and trees, and flowers are preferred.

In another embodiment of the invention host plants for the nucleic acid molecule, nucleic acid construct, expression cassette or vector according to the invention are selected from the group comprising corn, soy, oil seed rape (including canola and winter oil seed rape), cotton, wheat and rice.

A further object of the invention relates to the use of a nucleic acid construct, e.g. an expression cassette, comprising one or more nucleic acid molecules encoding one or more polypeptides as depicted in the respective line(s) in Table II, application no. 7, columns 5 or 8, or homologs or fragments thereof, or comprising one or more nucleic acid molecules as depicted in the respective line(s) in Table I, application no. 7, columns 5 or 8, preferably the coding region thereof, or homologs or fragments thereof, or comprising a nucleic acid molecule hybridizing therewith for the transformation of a microorganism or a plant cell, plant tissues or a part of a plant

In doing so, depending on the choice of promoter, the nucleic acid molecules shown in the respective line in Table I, application no. 7, column 5 or 8, preferably the coding region thereof, or homologs or fragments thereof, or the nucleic acid molecules encoding a polypeptide as depicted in the respective line in Table II, application no. 7, column 5 or 8, or homologs or fragments thereof, can be expressed in the whole plant or specifically e.g. in the leaves, in the seeds, the nodules, in roots, in the stem or other parts of the plant. Those transgenic plants overproducing sequences, e.g. as depicted in the respective line in Table I, application no. 7, column 5 or 8, preferably the coding region thereof, or homologs or fragments thereof, the plant cell, plant tissues, plants, the reproductive material thereof, or parts thereof are a further object of the present invention.

Within the framework of the present invention, the production of the alpha-tocopherol, or gamma-tocopherol is due to the generation or over-expression of one or more polypeptides as depicted in the respective line(s) in Table II, application no. 7, column 5 or 8, or homologs or fragments thereof, or encoded by the corresponding nucleic acid molecules as depicted in the respective line(s) in Table I, application no. 7, column 5 or 8, preferably the coding region thereof, or homologs or fragments thereof, in the non-human organism according to the invention, advantageously in the transgenic microorganism or plant according to the invention, at least for the duration of at least one plant generation.

A constitutive expression of the polypeptide as depicted in the respective line in Table II, application no. 7, column 5 or 8, or homologs or fragments thereof, or encoded by the respective nucleic acid molecule as depicted in the respective line in Table I, application no. 7, column 5 or 8, preferably the coding region thereof, or homologs or fragments thereof, may be advantageous. On the other hand, an inducible expression may also appear desirable. Expression of the polypeptide sequences of the invention can be either directed to the cytosol or to the organelles, such as plastids or mitochondria, preferably the plastids of the host cells, preferably the plant cells, or non-targeted.

The efficiency of the expression of the polypeptides depicted in the respective line in Table II, column 5 or 8, or homologs or fragments thereof, or encoded by the respective nucleic acid molecule as depicted in the respective line in Table I, column 5 or 8, preferably the coding region thereof, or homologs or fragments thereof, can be determined, for example, on test plants in greenhouse trials.

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 7, column 5 or 8, preferably shown in Table II A, application no. 7, column 5, or in Table II A, application no. 7, column 8, or in Table II B, application no. 7, column 8, or a homolog or a fragment thereof;
   (b) a nucleic acid molecule shown in Table I, application no. 7, column 5 or 8, preferably shown in Table I A, application no. 7, column 5, or in Table I A, application no. 7, column 8, or in Table I B, application no. 7, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 7, preferably shown in Table II A, application no. 7, column 5, or in Table II A, application no. 7, column 8, or in Table II B, application no. 7, column 8;
   (d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 7, preferably shown in Table I A, application no. 7, column 5, or in Table I A, application no. 7, column 8, or in Table I B, application no. 7, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 7;
   (i) a nucleic acid molecule encoding a polypeptide having the activity represented by a respective protein as depicted in column 5 of Table II, application no. 7;
   (j) a nucleic acid molecule comprising a polynucleotide which is obtained by amplifying a cDNA library or a genomic library using the primers in Table III, application no. 7, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising an expression cassette comprising
[0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 7, column 5 or 8, preferably shown in Table II A, application no. 7, column 5, or in Table II A, application no. 7, column 8, or in Table II B, application no. 7, column 8, or a homolog or a fragment thereof;
   (b) a nucleic acid molecule shown in Table I, application no. 7, column 5 or 8, preferably shown in Table I A, application no. 7, column 5, or in Table I A, application no. 7, column 8, or in Table I B, application no. 7, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 7, preferably shown in Table II A, application no. 7, column 5, or in Table II A, application no. 7, column 8, or in Table II B, application no. 7, column 8;
   (d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 7, preferably shown in Table I A, application no. 7, column 5, or in Table I A, application no. 7, column 8, or in Table I B, application no. 7, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 7;
   (i) a nucleic acid molecule encoding a polypeptide having the activity represented by a respective protein as depicted in column 5 of Table II, application no. 7;
   (j) a nucleic acid molecule comprising a polynucleotide which is obtained by amplifying a cDNA library or a genomic library using the primers in Table III, application no. 7, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide; which are operable linked;]
   or comprising or being transformed by a vector comprising
[1) a nucleic acid molecule selected from the group consisting of
   (a) a nucleic acid molecule encoding a polypeptide shown in Table II, application no. 7, column 5 or 8, preferably shown in Table II A, application no. 7, column 5, or in Ta ble II A, application no. 7, column 8, or in Table II B, application no. 7, column 8, or a homolog or a fragment thereof;
   (b) a nucleic acid molecule shown in Table I, application no. 7, column 5 or 8, preferably shown in Table I A, application no. 7, column 5, or in Table I A, application no. 7, column 8, or in Table I B, application no. 7, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 7, preferably shown in Table II A, application no. 7, column 5, or in Table II A, application no. 7, column 8, or in Table II B, application no. 7, column 8;
   (d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 7, preferably shown in Table I A, application no. 7, column 5, or in Table I A, application no. 7, column 8, or in Table I B, application no. 7, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 7;
   (i) a nucleic acid molecule encoding a polypeptide having the activity represented by a respective protein as depicted in column 5 of Table II, application no. 7;
   (j) a nucleic acid molecule comprising a polynucleotide which is obtained by amplifying a cDNA library or a genomic library using the primers in Table III, application no. 7, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 7.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 7 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 7, in the same line as of the respective nucleic acid molecule according to 1) “plastidic” is depicted, or encoding a mitochondrial transit peptide, especially in case in column 6 of Table I, application no. 7, in the same line as of the respective nucleic acid molecule according to 1) “mitochondric” is depicted.

In another embodiment of said transgenic non-human organism the expression cassette does not comprise 2) a nucleic acid molecule encoding a transit peptide, especially in case in column 6 of Table I, application no. 7, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 7.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 7, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 7, in the same line as of the respective nucleic acid molecule according to 1) “plastidic” is depicted, or encoding a mitochondrial transit peptide, especially in case in column 6 of Table I, application no. 7, in the same line as of the respective nucleic acid molecule according to 1) “mitochondric” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 7, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 7, in the same line as of the respective nucleic acid molecule according to 1) “plastidic” is depicted, or encoding a mitochondrial transit peptide, especially in case in column 6 of Table I, application no. 7, in the same line as of the respective nucleic acid molecule according to 1) “mitochondric” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 7, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 7, in the same line as of the respective nucleic acid molecule according to 1) “plastidic” is depicted, or encoding a mitochondrial transit peptide, especially in case in column 6 of Table I, application no. 7, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 7.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 7, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 7.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 7, in the same line as of the respective nucleic acid molecule according to 1) “plastidic” is depicted, or encoding a mitochondrial transit peptide, especially in case in column 6 of Table I, application no. 7, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 7, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 7, in the same line as of the respective nucleic acid molecule according to 1) “plastidic” is depicted, or encoding a mitochondrial transit peptide, especially in case in column 6 of Table I, application no. 7, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 7 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 7, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 7 (in the same line) and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 7, column 5 or 8, preferably shown in Table II A, application no. 7, column 5, or in Table II A, application no. 7, column 8, or in Table II B, application no. 7, column 8, or a homolog or a fragment thereof;
   (b) a nucleic acid molecule shown in Table I, application no. 7, column 5 or 8, preferably shown in Table I A, application no. 7, column 5, or in Table I A, application no. 7, column 8, or in Table I B, application no. 7, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 7, preferably shown in Table II A, application no. 7, column 5, or in Table II A, application no. 7, column 8, or in Table II B, application no. 7, column 8;
   (d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 7, preferably shown in Table I A, application no. 7, column 5, or in Table I A, application no. 7, column 8, or in Table I B, application no. 7, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 7;
   (i) a nucleic acid molecule encoding a polypeptide having the activity represented by a respective protein as depicted in column 5 of Table II, application no. 7;
   (j) a nucleic acid molecule comprising a polynucleotide which is obtained by amplifying a cDNA library or a genomic library using the primers in Table III, application no. 7, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide; which are operable linked;]
   or comprising an expression cassette comprising
[0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 7, column 5 or 8, preferably shown in Table II A, application no. 7, column 5, or in Table II A, application no. 7, column 8, or in Table II B, application no. 7, column 8, or a homolog or a fragment thereof;
   (b) a nucleic acid molecule shown in Table I, application no. 7, column 5 or 8, preferably shown in Table I A, application no. 7, column 5, or in Table I A, application no. 7, column 8, or in Table I B, application no. 7, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 7, preferably shown in Table II A, application no. 7, column 5, or in Table II A, application no. 7, column 8, or in Table II B, application no. 7, column 8;
   (d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 7, preferably shown in Table I A, application no. 7, column 5, or in Table I A, application no. 7, column 8, or in Table I B, application no. 7, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 7;
   (i) a nucleic acid molecule encoding a polypeptide having the activity represented by a respective protein as depicted in column 5 of Table II, application no. 7;
   (j) a nucleic acid molecule comprising a polynucleotide which is obtained by amplifying a cDNA library or a genomic library using the primers in Table III, application no. 7, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
   and, if desired
2) nucleic acid molecule encoding a transit peptide, preferably encoding a plastidal transit peptide or a mitochondrial transit peptide;
   which are operable linked;]
   or comprising or being transformed by a vector comprising [1) a nucleic acid molecule selected from the group consisting of
   (a) a nucleic acid molecule encoding a polypeptide shown in Table II, application no. 7, column 5 or 8, preferably shown in Table II A, application no. 7, column 5, or in Table II A, application no. 7, column 8, or in Table II B, application no. 7, column 8, or a homolog or a fragment thereof;
   (b) a nucleic acid molecule shown in Table I, application no. 7, column 5 or 8, preferably shown in Table I A, application no. 7, column 5, or in Table I A, application no. 7, column 8, or in Table I B, application no. 7, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 7, preferably shown in Table II A, application no. 7, column 5, or in Table II A, application no. 7, column 8, or in Table II B, application no. 7, column 8;
   (d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 7, preferably shown in Table I A, application no. 7, column 5, or in Table I A, application no. 7, column 8, or in Table I B, application no. 7, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 7;
   (i) a nucleic acid molecule encoding a polypeptide having the activity represented by a respective protein as depicted in column 5 of Table II, application no. 7;
   (j) a nucleic acid molecule comprising a polynucleotide which is obtained by amplifying a cDNA library or a genomic library using the primers in Table III, application no. 7, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      whereby, preferably, the nucleic acid molecule according to 1 (a), (b), (c), (d), (e), (f), (g), (h), (i), (j) or (k) distinguishes over the sequence depicted in Table I A and/or I B, application no. 7, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 7, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 7, column 5 or 8, or the coding region thereof, respectively. In a further embodiment the nucleic acid molecule does not encode the polypeptide sequence shown in Table II A and/or II B, application no. 7, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 7, column 5 or 8 and does not encode a protein of the sequence shown in Table II A and/or II B, application no. 7, column 5 or 8. Accordingly, in one embodiment, the protein encoded by a sequence of a nucleic acid according to 1 (a), (b), (c), (d), (e), (f), (g), (h), (i), (j) or (k) does not consist of the sequence shown in Table I A and/or I B, application no. 7, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 7, column 5 or 8 but less than 100%, preferably less than 99.999%, 99.99%, 99.9%, 99%, 98%, 97%, 96% or 95% identical to the sequence shown in Table II A and/or II B, application no. 7, column 5 or 8;
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 7.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 7 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 7, in the same line as of the respective nucleic acid molecule according to 1) “plastidic” is depicted, or encoding a mitochondrial transit peptide, especially in case in column 6 of Table I, application no. 7, in the same line as of the respective nucleic acid molecule according to 1) “mitochondric” is depicted.

In another embodiment thereof the expression cassette does not comprise 2) a nucleic acid molecule encoding a transit peptide, especially in case in column 6 of Table I, application no. 7, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 7.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the same line in column 5 of Table II, application no. 7, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 7, in the same line as of the respective nucleic acid molecule according to 1) “plastidic” is depicted, or encoding a mitochondrial transit peptide, especially in case in column 6 of Table I, application no. 7, in the same line as of the respective nucleic acid molecule according to 1) “mitochondric” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 7, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 7, in the same line as of the respective nucleic acid molecule according to 1) “plastidic” is depicted, or encoding a mitochondrial transit peptide, especially in case in column 6 of Table I, application no. 7, in the same line as of the respective nucleic acid molecule according to 1) “mitochondric” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 7, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 7, in the same line as of the respective nucleic acid molecule according to 1) “plastidic” is depicted, or encoding a mitochondrial transit peptide, especially in case in column 6 of Table I, application no. 7, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 7.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 7, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 7.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 7, in the same line as of the respective nucleic acid molecule according to 1) “plastidic” is depicted, or encoding a mitochondrial transit peptide, especially in case in column 6 of Table I, application no. 7, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 7, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 7, in the same line as of the respective nucleic acid molecule according to 1) “plastidic” is depicted, or encoding a mitochondrial transit peptide, especially in case in column 6 of Table I, application no. 7, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 7 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 7, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 7 and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

In an embodiment thereof the transgenic non-human organism is a microorganism selected from the group consisting of Charophyceae such as the genera Chara, Nitella e.g. the species Chara globularis, Chara vulgaris, Nitella flexilis, Chlorophyceae such as the genera Acrosiphonia, Spongomorpha, Urospora, Bryopsis, Pseudobryopsis, Trichosolen, Dichotomosiphon, Caulerpa, Rhipilia, Blastophysa, Avrainvillea, Chlorodesmis, Codium, Espera, Halicystis, Halimeda, Penicillus, Pseudocodium, Rhipiliopsis, Rhipocephalus, Tydemania, Udotea, Derbesia, Acrochaete, Aphanochaete, Bolbocoleon, Chaetobolus, Chaetonema,

Chaetophora, Chlorotylium, Desmococcus, Draparnaldia, Draparnaldiopsis, Ectochaete, Endophyton, Entocladia, Epicladia, Internoretia, Microthamnion, Ochlochaete, Phaeophila, Pilinella, Pringsheimiella, Protoderma, Pseudendoclonium, Pseudodictyon, Pseudopringsheimia, Pseudulvella, Schizomeris, Stigeoclonium, Thamniochaete, Ulvella, Pilinia, Tellamia, Helicodictyon, Actidesmium, Ankyra, Characium, Codiolum, Sykidion, Keratococcus, Prototheca, Bracteacoccus Chlorococcum, Excentrosphaera, Hormidium, Oophila, Schroederia, Tetraedron, Trebouxia Chlorosarcinopsis, Gomphonitzschia, Coccomyxa, Dactylothece, Diógenes, Disporá, Gloeocystis, Mycanthococcus, Ourococcus, Coelastrum, Dicranochaete, Botryococcus, Dictyosphaerium, Dimorphococcus, Chlorochytrium, Kentrosphaera, Phyllobium, Gomontia, Hormotila, Euastropsis, Hydrodictyon, Pectodictyon, Pediastrum, Sorastrum, Tetrapedia, Acanthosphaera, Echinosphaerella, Echinosphaeridium, Errerella, Gloeoactinium, Golenkeniopsis Golenkinia, Micractinium, Ankistrodesmus, Chlorella, Chodatella, Closteriopsis, Cryocystis, Dactylococcus, Dematractum, Eremosphaera, Eutetramorus, Franceia, Glaucocystis, Gloeotaenium, Kirchneriella, Lagerheimiella, Monoraphidium, Nannochloris, Nephrochlamys, Nephrocytium, Oocystis, Oonephris, Pachycladon, Palmellococcus, Planktosphaeria, Polyedriopsis, Pseudoraciborskia, Quadrigula, Radiococcus, Rochiscia, Scotiella, Selanastrum, Thorakochloris, Treubaria, Trochiscia, Westella, Zoochlorella, Ostreobium, Phyllosiphon, Protosiphon, Rhodochytrium, Actinastrum, Coronastrum, Crucigenia, Dictymocystis, Enallax, Scenedesmus, Selenastrum, Tetradesmus, Tetrallantos, Tetrastrum, Chlorosarcina, Anadyomene, Valoniopsis, Ventricaria, Basicladia, Chaetomorpha, Cladophora, LolaPithophoraRhizoclonium Chaetosphaeridium, Conochaete, Coleochaete, Oligochaetophora, Polychaetophora, Cylindrocapsa, Gongrosira, Protococcus, Acetabularia, Batophora, Bornetella, Dasycladus, Halicoryne, Neomeris, Elakatothrix, Raphidonema, Microspora, Bulbochaete, Oedocladium, Oedogonium, Prasiola, Rosenvingiella, Schizogonium, Apjohnia, Chamaedoris, Cladophoropsis, Siphonocladus, Spongocladia, Boergesenia, Boodlea, Cystodictyon, Dictyosphaeria, Ernodesmis, Microdictyon, Struvea, Valonia, Sphaeroplea, Malleochloris, Stylosphaeridium, Gloeococcus, Palmella, Palmodictyon, Palmophyllum, Pseudospherocystis, Sphaerocystis, Urococcus, Apiocystis, Chaetopeltis, Gemellicystis, Paulschulzia, Phacomyxa, Pseudotetraspora, Schizochlamys, Tetraspora, Cephaleuros, Ctenocladus, Epibolium, Leptosira, Trentepohlia, Diplochaete, Monostroma, Binuclearia, Geminella, Klebsormidium, Planetonema, Radiofilum, Stichococcus, Ulothrix, Uronema, Blidingia, Capsosiphon, Chloropelta, Enteromorpha, Percursaria, Ulva, Ulvaria, Brachiomonas, Carteria, Chlainomonas, Chlamydomonas, Chlamydonephris, Chlorangium, Chlorogonium, Cyanidium, Fortiella, Glenomonas, Gloeomonas, Hyalogonium, Lobomonas Polytoma, Pyramichlamys, Scourfieldia, Smithsonimonas, Sphaerellopsis, Sphenochloris, Spirogonium, Collodictyon, Dunaliella, Haematococcus, Stephanosphaera, Coccomonas, Dysmorphococcus, Phacotus, Pteromonas, Thoracomonas, Wislouchiella, Mascherina, Pyrobotrys, Spondylomorum, Eudorina, Gonium, Oltmannsiella, Pandorina, Platydorina, Pleodorina, Stephanoon, Volvox, Volvulina, Actinotaenium, Arthrodesmus, Bambusina Closterium, Cosmarium, Desmidium, Euastrum, Groenbladia, Hyalotheca, Micrasterias, Penium, Phymatodocis, Pleurotaenium, Sphaerozosma, Spinoclosterium, Spinocosmarium, Spondylosium, Staurastrum, Tetmemorus, Triploceras, Xanthidium, Cylindrocystis, Genicularia, Gonatozygon, Mesotaenium, Netrium, Roya, Spirotaenia, Cosmocladium, Debarya, Docidium, Euastridium, Hallasia, Mougeotia, Mougeotiopsis, Sirogonium, Spirogyra, Staurodesmus, Teilingia, Zygnema, Zygogonium, e.g. the species Caulerpa taxifolia, Prototheca wickerhamii, Ankistrodesmus falcatus, Chlorella ellipsoidea, Chlorella pyrenoidosa, Clorella sorokiniana, Chlorella vulgaris, Scenedesmus obliquus, Scenedesmus quadricauda, Selenastrum capricornutum, Selenastrum undecimnotata, Cladophora glomerata, Chlamydomonas eugametos, Chlamydomonas reinhardtii, Cyanidium caldarium, Dunaliella salina, Dunaliella tertiolecta, Euglena gracilis, Haematococcus pluvialis, Coniugatophyceae, Prasinophyceae Trebouxiophyceae, Ulvophyceae, Chlorodendraceae, Pedinomonadales, Halosphaeraceae, Pterospermataceae, Monomastigaceae, Pyramimonadaceae, Chlorodendraceae such as the genera Prasinocladus e.g. the species Prasinocladus ascus, Halosphaeraceae, Pedinomonadales,Pedinomonadaceae such as the genera Pedinomonas, Pterospermataceae such as the genera Pachysphaera, Pterosperma, Halosphaera, Pyramimonas, Bacillariophyceae, Chrysophyceae, Craspedophyceae, Euglenophyceae, Prymnesiophyceae, Phaeophyceae, Dinophyceae, Rhodophyceae, Xanthophyceae, Prasinophyceae such as the genera Nephroselmis, Prasinococcus, Scherffelia, Tetraselmis, Mantoniella, Ostreococcus e.g. the species Nephroselmis olivacea, Prasinococcus capsulatus, Scherffelia dubia, Tetraselmis chui, Tetraselmis suecica, Mantoniella squamata or Ostreococcus tauri.

In an embodiment thereof the transgenic non-human organism is a plant (or a part thereof), preferably a monocotyledonous or a dicotyledonous plant. Preferably the plant is selected from the group consisting of Anacardiaceae such as the genera Pistacia, Mangifera, Anacardium e.g. the species Pistacia vera [pistachios, Pistazie], Mangifer indica [Mango] or Anacardium occidentale [Cashew]; Asteraceae such as the genera Calendula, Carthamus, Centaurea, Cichorium, Cynara, Helianthus, Lactuca, Locusta, Tagetes, Valeriana e.g. the species Calendula officinalis [Marigold], Carthamus tinctorius [safflower], Centaurea cyanus [cornflower], Cichorium intybus [blue daisy], Cynara scolymus [Artichoke], Helianthus annus [sunflower], Lactuca sativa, Lactuca crispa, Lactuca esculenta, Lactuca scariola L. ssp. sativa, Lactuca scariola L. var. integrate, Lactuca scariola L. var. integrifolia, Lactuca sativa subsp. romana, Locusta communis, Valeriana locusta [lettuce], Tagetes lucida, Tagetes erecta or Tagetes tenuifolia [Marigold]; Apiaceae such as the genera Daucus e.g. the species Daucus carota [carrot]; Betulaceae such as the genera Corylus e.g. the species Corylus avellana or Corylus colurna [hazelnut]; Boraginaceae such as the genera Borago e.g. the species Borago officinalis [borage]; Brassicaceae such as the genera Brassica, Melanosinapis, Sinapis, Arabadopsis e.g. the species Brassica napus, Brassica rapa ssp. [canola, oilseed rape, turnip rape], Sinapis arvensis Brassica juncea, Brassica juncea var. juncea, Brassica juncea var. crispifolia, Brassica juncea var. foliosa, Brassica nigra, Brassica sinapioides, Melanosinapis communis [mustard], Brassica oleracea [fodder beet] or Arabidopsis thaliana; Bromeliaceae such as the genera Anana, Bromelia e.g. the species Anana comosus, Ananas ananas or Bromelia comosa [pineapple]; Caricaceae such as the genera Carica e.g. the species Carica papaya [papaya]; Cannabaceae such as the genera Cannabis e.g. the species Cannabis sative [hemp], Convolvulaceae such as the genera Ipomea, Convolvulus e.g. the species Ipomoea batatus, Ipomoea pandurata, Convolvulus batatas, Convolvulus tiliaceus, Ipomoea fastigiata, Ipomoea tiliacea, Ipomoea triloba or Convolvulus panduratus [sweet potato, Man of the Earth, wild potato], Chenopodiaceae such as the genera Beta, i.e. the species Beta vulgaris, Beta vulgaris var. altissima, Beta vulgaris var. Vulgaris, Beta maritima, Beta vulgaris var. perennis, Beta vulgaris var. conditiva or Beta vulgaris var. esculenta [sugar beet]; Cucurbitaceae such as the genera Cucubita e.g. the species Cucurbita maxima, Cucurbita mixta, Cucurbita pepo or Cucurbita moschata [pumpkin, squash]; Elaeagnaceae such as the genera Elaeagnus e.g. the species Olea europaea [olive]; Ericaceae such as the genera Kalmia e.g. the species Kalmia latifolia, Kalmia angustifolia, Kalmia microphylla, Kalmia polifolia, Kalmia occidentalis, Cistus chamaerhodendros or Kalmia lucida [American laurel, broad-leafed laurel, calico bush, spoon wood, sheep laurel, alpine laurel, bog laurel, western bog-laurel, swamp-laurel]; Euphorbiaceae such as the genera Manihot, Janipha, Jatropha, Ricinus e.g. the species Manihot utilissima, Janipha manihot, Jatropha manihot, Manihot aipil, Manihot dulcis, Manihot manihot, Manihot melanobasis, Manihot esculenta [manihot, arrowroot, tapioca, cassava] or Ricinus communis [castor bean, Castor Oil Bush, Castor Oil Plant, Palma Christi, Wonder Tree]; Fabaceae such as the genera Pisum, Albizia, Cathormion, Feuillea, Inga, Pithecolobium, Acacia, Mimosa, Medicajo, Glycine, Dolichos, Phaseolus, Soja e.g. the species Pisum sativum, Pisum arvense, Pisum humile [pea], Albizia berteriana, Albizia julibrissin, Albizia lebbeck, Acacia berteriana, Acacia littoralis, Albizia berteriana, Albizzia berteriana, Cathormion berteriana, Feuillea berteriana, Inga fragrans, Pithecellobium berterianum, Pithecellobium fragrans, Pithecolobium berterianum, Pseudalbizzia berteriana, Acacia julibrissin, Acacia nemu, Albizia nemu, Feuilleea julibrissin, Mimosa julibrissin, Mimosa speciosa, Sericanrda julibrissin, Acacia lebbeck, Acacia macrophylla, Albizia lebbek, Feuilleea lebbeck, Mimosa lebbeck, Mimosa speciosa [bastard logwood, silk tree, East Indian Walnut], Medicago sativa, Medicago falcata, Medicago varia [alfalfa], Glycine max [soybean], Dolichos soja, Glycine gracilis, Glycine hispida, Phaseolus max or Soja hispida; Geraniaceae such as the genera Pelargonium, Cocos, Oleum e.g. the species Cocos nucifera, Pelargonium grossularioides or Oleum cocois [coconut]; Gramineae such as the genera Saccharum e.g. the species Saccharum officinarum; Juglandaceae such as the genera Juglans, Wallia e.g. the species Juglans regia, Juglans ailanthifolia, Juglans sieboldiana, Juglans cinerea, Wallia cinerea, Juglans bixbyi, Juglans californica, Juglans hindsii, Juglans intermedia, Juglans jamaicensis, Juglans major, Juglans microcarpa, Juglans nigra or Wallia nigra [walnut, black walnut, common walnut, persian walnut, white walnut, butternut, black walnut]; Lauraceae such as the genera Persea, Laurus e.g. the species laurel Laurus nobilis [bay, laurel, bay laurel, sweet bay], Persea americana Persea americana, Persea gratissima or Persea persea [avocado]; Leguminosae such as the genera Arachis e.g. the species Arachis hypogaea [peanut]; Linaceae such as the genera Linum, Adenolinum e.g. the species Linum usitatissimum, Linum humile, Linum austriacum, Linum bienne, Linum angustifolium, Linum catharticum, Linum flavum, Linum grandiflorum, Adenolinum grandiflorum, Linum lewisii, Linum narbonense, Linum perenne, Linum perenne var. lewisii, Linum pratense or Linum trigynum [flax, linseed]; Lythrarieae such as the genera Punica e.g. the species Punica granatum [pomegranate]; Malvaceae such as the genera Gossypium e.g. the species Gossypium hirsutum, Gossypium arboreum, Gossypium barbadense, Gossypium herbaceum or Gossypium thurberi [cotton]; Musaceae such as the genera Musa e.g. the species Musa nana, Musa acuminata, Musa paradisiaca, Musa spp. [banana]; Onagraceae such as the genera Camissonia, Oenothera e.g. the species Oenothera biennis or Camissonia brevipes [primrose, evening primrose]; Palmae such as the genera Elacis e.g. the species Elaeis guineensis [oil plam]; Papaveraceae such as the genera Papaver e.g. the species Papaver orientale, Papaver rhoeas, Papaver dubium [poppy, oriental poppy, corn poppy, field poppy, shirley poppies, field poppy, long-headed poppy, long-pod poppy]; Pedaliaceae such as the genera Sesamum e.g. the species Sesamum indicum [sesame]; Piperaceae such as the genera Piper, Artanthe, Peperomia, Steffensia e.g. the species Piper aduncum, Piper amalago, Piper angustifolium, Piper auritum, Piper betel, Piper cubeba, Piper longum, Piper nigrum, Piper retrofractum, Artanthe adunca, Artanthe elongata, Peperomia elongata, Piper elongatum, Steffensia elongata. [Cayenne pepper, wild pepper]; Poaceae such as the genera Hordeum, Secale, Avena, Sorghum, Andropogon, Holcus, Panicum, Oryza, Zea, Triticum e.g. the species Hordeum vulgare, Hordeum jubatum, Hordeum murinum, Hordeum secalinum, Hordeum distichon Hordeum aegiceras, Hordeum hexastichon, Hordeum hexastichum, Hordeum irregulare, Hordeum sativum, Hordeum secalinum [barley, pearl barley, foxtail barley, wall barley, meadow barley], Secale cereale [rye], Avena sativa, Avena fatua, Avena byzantina, Avena fatua var. sativa, Avena hybrida [oat], Sorghum bicolor, Sorghum halepense, Sorghum saccharatum, Sorghum vulgare, Andropogon drummondii, Holcus bicolor, Holcus sorghum, Sorghum aethiopicum, Sorghum arundinaceum, Sorghum caffrorum, Sorghum cernuum, Sorghum dochna, Sorghum drummondii, Sorghum durra, Sorghum guineense, Sorghum lanceolatum, Sorghum nervosum, Sorghum saccharatum, Sorghum subglabrescens, Sorghum verticilliflorum, Sorghum vulgare, Holcus halepensis, Sorghum miliaceum millet, Panicum militaceum [Sorghum, millet], Oryza sativa, Oryza latifolia [rice], Zea mays [corn, maize] Triticum aestivum, Triticum durum, Triticum turgidum, Triticum hybernum, Triticum macha, Triticum sativum or Triticum vulgare [wheat, bread wheat, common wheat], Proteaceae such as the genera Macadamia e.g. the species Macadamia intergrifolia [macadamia]; Rubiaceae such as the genera Coffea e.g. the species Cofea spp., Coffea arabica, Coffea canephora or Coffea liberica [coffee]; Scrophulariaceae such as the genera Verbascum e.g. the species Verbascum blattaria, Verbascum chaixii, Verbascum densiflorum, Verbascum lagurus, Verbascum longifolium, Verbascum lychnitis, Verbascum nigrum, Verbascum olympicum, Verbascum phlomoides, Verbascum phoenicum, Verbascum pulverulentum or Verbascum thapsus [mullein, white moth mullein, nettle-leaved mullein, dense-flowered mullein, silver mullein, long-leaved mullein, white mullein, dark mullein, greek mullein, orange mullein, purple mullein, hoary mullein, great mullein]; Solanaceae such as the genera Capsicum, Nicotiana, Solanum, Lycopersicon e.g. the species Capsicum annuum, Capsicum annuum var. glabriusculum, Capsicum frutescens [pepper], Capsicum annuum [paprika], Nicotiana tabacum, Nicotiana alata, Nicotiana attenuata, Nicotiana glauca, Nicotiana langsdorffii, Nicotiana obtusifolia, Nicotiana quadrivalvis, Nicotiana repanda, Nicotiana rustica, Nicotiana sylvestris [tobacco], Solanum tuberosum [potato], Solanum melongena [egg-plant] (Lycopersicon esculentum, Lycopersicon lycopersicum, Lycopersicon pyriforme, Solanum integrifolium or Solanum lycopersicum [tomato]; Sterculiaceae such as the genera Theobroma e.g. the species Theobroma cacao [cacao]; Theaceae such as the genera Camellia e.g. the species Camellia sinensis) [tea]

Particular preference is given to transgenic crop plants such as by way of example barley, wheat, rye, oats, corn, soybean, rice, cotton, sugar beet, oilseed rape and canola, sunflower, flax, hemp, thistle, potatoes, tobacco, tomatoes, tapioca, cassava, arrowroot, alfalfa, lettuce and the various tree, nut and vine species; especial preference is given to monocotyledonous crop plants like corn, wheat or rice; in another embodiment special preference is given to dicotyledonous crop plants like soy bean, oil seed rape (including canola and winter oil seed rape), cotton.

The term “transgenic plants” used in accordance with the invention also refers to the progeny of a transgenic plant, for example the T₁, T₂, T₃ and subsequent plant generations or the BC₁, BC₂, BC₃ and subsequent plant generations. Thus, the transgenic plants according to the invention can be raised and selfed or crossed with other individuals in order to obtain further transgenic plants according to the invention. Transgenic plants may also be obtained by propagating transgenic plant cells vegetatively. The present invention also relates to transgenic plant material, which can be derived from a transgenic plant population according to the invention. Such material includes plant cells and certain tissues, organs and parts of plants in all their manifestations, such as seeds, leaves, anthers, fibers, tubers, roots, root hairs, stems, embryo, calli, cotyledons, petioles, harvested material, plant tissue, reproductive tissue, pollen, and cell cultures, which are derived from the actual transgenic plant and/or can be used for bringing about the transgenic plant. Any transformed plant obtained according to the invention can be used in a conventional breeding scheme or in vitro plant propagation to produce more transformed plants with the same characteristics and/or can be used to introduce the same characteristic in other varieties of the same or related species. Such plants are also part of the invention. Seeds obtained from the transformed plants genetically also contain the same characteristic and are part of the invention. As mentioned before, the present invention is in principle applicable to any plant and crop that can be transformed with any of the transformation method known to those skilled in the art.

An “isolated” nucleic acid molecule is one that is substantially separated from other nucleic acid molecules, which are present in the natural source of the nucleic acid. That means other nucleic acid molecules are present in an amount less than 5% based on weight of the amount of the desired nucleic acid, preferably less than 2% by weight, more preferably less than 1% by weight, most preferably less than 0.5% by weight. Preferably, an “isolated” nucleic acid is free of some of the sequences that naturally flank the nucleic acid (i.e., sequences located at the 5′ and 3′ ends of the nucleic acid) in the genomic DNA of the non-human organism from which the nucleic acid is derived. For example, in various embodiments, the FCRP encoding nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be free from some of the other cellular material with which it is naturally associated, or culture medium when produced by recombinant techniques, or chemical precursors or other chemicals when chemically synthesized.

A nucleic acid molecule of the present invention, e.g., a nucleic acid molecule encoding a FCRP, especially the coding region thereof, or a portion thereof which confers the production or increased production of the fine chemical, can be isolated using standard molecular biological techniques and the sequence information provided herein. For example, an A. thaliana FCRP encoding cDNA can be isolated from a A. thaliana c-DNA library or a E. coli, Saccharomyces cerevisiae, Synechocystis sp., Brassica napus, Glycine max, Zea mays or Oryza sativa FCRP encoding cDNA can be isolated from a E. coli, Saccharomyces cerevisiae, Synechocystis sp., Brassica napus, Glycine max, Zea mays or Oryza sativa c-DNA library respectively using all or portion of one of the respective sequences. Moreover, a nucleic acid molecule encompassing all or a portion of one of the sequences of Table I can be isolated by the polymerase chain reaction using oligonucleotide primers designed based upon this sequence. For example, mRNA can be isolated from plant cells (e.g., by the guanidiniumthiocyanate extraction procedure of Chirgwin et al., Biochemistry 18, 5294 (1979)) and cDNA can be prepared using reverse transcriptase (e.g., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for polymerase chain reaction amplification can be designed based upon one of the nucleotide sequences shown in Table I. A nucleic acid molecule of the invention can be amplified using cDNA or, alternatively, genomic DNA, as a template and appropriate oligonucleotide primers according to standard PCR amplification techniques. The nucleic acid molecule so amplified can be cloned into an appropriate vector and characterized by DNA sequence analysis. Furthermore, oligonucleotides corresponding to a FCRP encoding nucleotide sequence can be prepared by standard synthetic techniques, e.g., using an automated DNA synthesizer.

In an embodiment, an isolated nucleic acid molecule of the invention comprises one of the nucleic acid molecules as shown in Table I, especially the coding region thereof, and if desired, as well as a 5′ untranslated sequence and 3′ untranslated sequence.

Moreover, the nucleic acid molecule of the invention can comprise only a portion of the coding region of one of the nucleic acid molecule of Table I, for example, a fragment which can be used as a probe or primer or a fragment encoding a biologically active portion of a FCRP.

Portions of proteins encoded by the FCRP encoding nucleic acid molecules of the invention are preferably biologically active portions described herein. As used herein, the term “biologically active portion of” a FCRP is intended to include a portion, e.g. a domain/motif, of the protein being responsible for the ability of the protein to enable the production or increased production of the fine chemical. To determine whether a FCRP, or a biologically active portion thereof, results in a production or an increased production of the fine chemical in the non-human organism, like a microorganism or a plant, an analysis of the non-human organism comprising the FCRP may be performed. Such analysis methods are well known to those skilled in the art, as detailed in the Examples. More specifically, nucleic acid fragments encoding biologically active portions of a FCRP can be prepared by isolating a portion of one of the sequences of the nucleic acid of Table I, e.g. expressing the encoded portion of the FCRP or peptide (e.g. by recombinant expression in vitro) and assessing the activity of the encoded portion of the FCRP or peptide.

Biologically active portions of a FCRP are encompassed by the present invention and include peptides comprising amino acid sequences derived from the amino acid sequence of a FCRP encoding gene, or the amino acid sequence of a protein homologous to a FCRP, which include fewer amino acids than a full length FCRP or the full length protein which is homologous to a FCRP, and exhibits at least some enzymatic or biological activity of a FCRP. Typically, biologically active portions (e.g., peptides which are, for example, 5, 10, 15, 20, 30, 35, 36, 37, 38, 39, 40, 50, 75, 100, 125, 150 or more amino acids in length) comprise a domain or motif with at least one activity of a FCRP. Moreover, other biologically active portions in which other regions of the protein are deleted, can be prepared by recombinant techniques and evaluated for one or more of the activities described herein. Preferably, the biologically active portions of a FCRP include one or more selected domains/motifs or portions thereof having biological activity.

The term “biological active portion” or “biological activity” means a polypeptide as depicted in the respective line in Table II, application no. 7, column 3 or a portion of said polypeptide which still has at least 10% or 20%, preferably 30%, 40%, 50% or 60%, especially preferably 70%, 75%, 80%, 90% or 95% of the enzymatic or biological activity of the natural or starting enzyme or protein.

In the process according to the invention nucleic acid sequences or molecules can be used, which, if appropriate, contain synthetic, non-natural or modified nucleotide bases, which can be incorporated into DNA or RNA. Said synthetic, non-natural or modified bases can for example increase the stability of the nucleic acid molecule outside or inside a cell. The nucleic acid molecules of the invention can contain the same modifications as aforementioned.

As used in the present context the term “nucleic acid molecule” may also encompass the untranslated sequence or molecule located at the 3′ and at the 5′ end of the coding gene region, for example at least 500, preferably 200, especially preferably 100, nucleotides of the sequence upstream of the 5′ end of the coding region and at least 100, preferably 50, especially preferably 20, nucleotides of the sequence downstream of the 3′ end of the coding gene region. It is often advantageous only to choose the coding region for cloning and expression purposes.

Preferably, the nucleic acid molecule used in the process according to the invention or the nucleic acid molecule of the invention is an isolated nucleic acid molecule. In one embodiment, the nucleic acid molecule of the invention is the nucleic acid molecule used in the process of the invention.

The nucleic acid molecules used in the process, for example the polynucleotide of the invention or of a part thereof can be isolated using molecular-biological standard techniques and the sequence information provided herein. Also, for example a homologous sequence or homologous, conserved sequence regions at the DNA or amino acid level can be identified with the aid of comparison algorithms. The former can be used as hybridization probes under standard hybridization techniques (for example those described in Sambrook et al., “Molecular Cloning: A Laboratory Manual” 2nd ed., Cold Spring Harbor Laboratory, Cold Spring Harbor Laboratory Press, Cold Spring Harbor, N.Y., 1989) for isolating further nucleic acid sequences useful in this process.

Synthetic oligonucleotide primers for the amplification, e.g. as shown in the respective line in Table III, application no. 7, column 8, by means of polymerase chain reaction can be generated on the basis of a sequence shown herein, for example the sequence shown in the respective line in Table I, application no. 7, columns 5 or 8, or the sequences derived from Table II, application no. 7, columns 5 or 8, respectively.

Moreover, it is possible to identify a conserved region of a protein from various organism by carrying out protein sequence alignments with the polypeptide encoded by the nucleic acid molecules of the present invention, in particular with the sequences encoded by the nucleic acid molecule shown in the respective line in column 5 or 8 of Table I, application no. 7, from which conserved regions, and in turn, degenerate primers can be derived. Conserved regions are those, which show a very little variation in the amino acid in one particular position of several homologs from different origin. The consensus sequence and polypeptide motifs shown in the respective line in column 8 of Table IV, application no. 7, are derived from such alignments of homologous proteins.

In an embodiment of the present invention, in the method of the present invention the activity of a polypeptide comprising or consisting of a consensus sequence or at least one polypeptide motif shown in the respective line in Table IV, application no. 7, column 8, is increased.

In another embodiment thereof, the method of the present invention relates to a polypeptide comprising or consisting of a consensus sequence shown in the respective line in Table IV, application no. 7, column 8 or comprising at least one polypeptide motif shown in the respective line in Table IV, application no. 7, column 8, whereby not more than 20, 15, 10, 9, 8, 7, 6, 5 4, 3, 2 or 1, or 0 of the amino acid positions indicated can be replaced by any amino acid.

In another embodiment thereof, the method of the present invention relates to a polypeptide comprising or consisting of a consensus sequence shown in the respective line in Table IV, application no. 7, column 8 or comprising at least one polypeptide motif shown in the respective line in Table IV, application no. 7, column 8, whereby not more than 15%, 10%, 5%, 4%, 3%, 2% or 1%, or 0% of the amino acid positions defined as distinct specific amino acids are/is replaced by another amino acid.

In another embodiment thereof, the method of the present invention relates to a polypeptide comprising or consisting of a consensus sequence shown in the respective line in Table IV, application no. 7, column 8 or comprising at least one polypeptide motif shown in the respective line in Table IV, application no. 7, column 8, whereby not more than 20, 15, 10, 9, 8, 7, 6, 5, 4, 3, 2 or 1, or 0 amino acids are inserted into a consensus sequence or polypeptide motif.

In an embodiment of the present invention, in the method of the present invention the activity of a polypeptide comprising or consisting of a consensus sequence or at least one polypeptide motif shown in the respective line in Table IV, application no. 7, column 8, is increased, whereby said polypeptide distinguishes over the sequence depicted in Table II, application no. 7, columns 5 or 8 by one or more amino acids.

In one embodiment, said polypeptide of the invention distinguishes over the sequence shown in the respective line in Table II, application no. 7, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II B, application no. 7, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in Table II, application no. 7, columns 5 or 8.

The consensus sequence was derived from a multiple alignment of the sequences as listed in Table II. The amino acids are given in their three letter code. In case a specific amino acid is indicated this specific amino acid is conserved in at least 80% of the aligned proteins whereas the three letter code XAA stands for amino acids, which are not conserved in at least 80% of the aligned sequences. The consensus sequence starts with the first conserved amino acid in the alignment, and ends with the last conserved amino acid in the alignment of the investigated sequences.

Patterns had to match at least 80% of the investigated proteins. Conserved patterns were identified with the software tool MEME version 3.5.1 or manually by using first standard prosite annotation. The number of given x indicates the distances between conserved amino acid residues (given in the one letter code), e.g. (1) the pattern Y-x(21,23)-F means that conserved tyrosine and phenylalanine residues in the alignment are separated from each other by minimum 21 and maximum 23 amino acid residues in the alignment of all investigated sequences, (2) the pattern Y-x(21,23)-[FW] means that a conserved tyrosine is separated by minimum 21 and maximum 23 amino acid residues from either a phenylalanine or tryptophane. However, these patterns have been “translated” into the WIPO standard 25. MEME was developed by Bailey Timothy L. and Elkan Charles, Dept. of Computer Science and Engineering, University of California, San Diego, USA and is described by Bailey Timothy L. and Elkan Charles (Proceedings of the Second International Conference on Intelligent Systems for Molecular Biology, pp. 28-36, AAAI Press, Menlo Park, Calif., 1994). The source code for the stand-alone program is public available from the San Diego Supercomputer centre (http://meme.sdsc.edu). For identifying common motifs in all sequences with the software tool MEME, the following settings were used:—maxsize 500000,—nmotifs 15,—evt 0.001,—maxw 60,—distance 1e−3,—minsites number of sequences used for the analysis. Input sequences for MEME were non-aligned sequences in Fasta format. Other parameters were used in the default settings in this software version. Prosite patterns for conserved domains were generated with the software tool Pratt version 2.1 or manually. Pratt was developed by Jonassen Inge, Dept. of Informatics, University of Bergen, Norway and is described by Jonassen et al. (Jonassen I., Collins J. F. and Higgins D. G., Protein Science 4, 1587 (1995); Jonassen I., Efficient discovery of conserved patterns using a pattern graph, Submitted to CABIOS Febr. 1997). The source code (ANSI C) for the stand-alone program is public available, e.g. at established Bioinformatic centers like EBI (European Bioinformatics Institute). For generating patterns with the software tool Pratt, following settings were used: PL (max Pattern Length): 100, PN (max Nr of Pattern Symbols): 100, PX (max Nr of consecutive x's): 30, FN (max Nr of flexible spacers): 5, FL (max Flexibility): 30, FP (max Flex.Product): 10, ON (max number patterns): 50. Input sequences for Pratt were distinct regions of the protein sequences exhibiting high similarity as identified from software tool MEME. The minimum number of sequences, which have to match the generated patterns (CM, min Nr of Seqs to Match) was set to at least 80% of the provided sequences. Parameters not mentioned here were used in their default settings. The Prosite patterns of the conserved domains can be used to search for protein sequences matching this pattern. Various established Bioinformatic centres provide public internet portals for using those patterns in database searches (e.g. PIR (Protein Information Resource, located at Georgetown University Medical Center) or ExPASy (Expert Protein Analysis System)). Alternatively, stand-alone software is available, like the program Fuzzpro, which is part of the EMBOSS software package.

For example, the program Fuzzpro not only allows searching for an exact pattern-protein match but also allows setting various ambiguities in the performed search.

The alignment was performed with the software ClustalW (version 1.83) and is described by Thompson et al. (Nucleic Acids Research 22, 4673 (1994)). The source code for the stand-alone program is public available from the European Molecular Biology Laboratory; Heidelberg, Germany. The analysis was performed using the default parameters of ClustalW v1.83 (gap open penalty: 10.0; gap extension penalty: 0.2; protein matrix: Gonnet; protein/DNA endgap: −1; protein/DNA gapdist: 4).

Degenerated primers can then be utilized by PCR for the amplification of fragments of novel proteins having above-mentioned activity, e.g. conferring the production or the increased production of the fine chemical as compared to a corresponding, non-transformed, wild type cell, or non-human organism, like a plant cell or a plant or a part thereof, after generating or increasing the expression or activity or having the activity of a protein as shown in the respective line in Table II, column 3 or further functional homologs of the polypeptide of the invention from other non-human organisms.

These fragments can then be utilized as hybridization probe for isolating the complete gene sequence. As an alternative, the missing 5′ and 3′ sequences can be isolated by means of RACE-PCR. A nucleic acid molecule according to the invention can be amplified using cDNA or, as an alternative, genomic DNA as template and suitable oligonucleotide primers, following standard PCR amplification techniques. The nucleic acid molecule amplified thus can be cloned into a suitable vector and characterized by means of DNA sequence analysis. Oligonucleotides, which correspond to one of the nucleic acid molecules used in the process, can be generated by standard synthesis methods, for example using an automatic DNA synthesizer.

Nucleic acid molecules which are advantageously for the process according to the invention can be isolated based on their homology to the nucleic acid molecules disclosed herein using the sequences or part thereof as or for the generation of a hybridization probe and following standard hybridization techniques under stringent hybridization conditions. In this context, it is possible to use, for example, isolated one or more nucleic acid molecules of at least 15, 20, 25, 30, 35, 40, 50, 60 or more nucleotides, preferably of at least 15, 20 or 25 nucleotides in length which hybridize under stringent conditions with the above-described nucleic acid molecules, in particular with those which encompass a nucleotide sequence of the nucleic acid molecule used in the process of the invention or encoding a protein used in the invention or of the nucleic acid molecule of the invention. Nucleic acid molecules with 30, 50, 100, 250 or more nucleotides may also be used.

The term “homology” means that the respective nucleic acid molecules or the encoded proteins are functionally and/or structurally equivalent. The nucleic acid molecules that are homologous to the nucleic acid molecules described above and that are derivatives of said nucleic acid molecules are, for example, variations of said nucleic acid molecules which represent modifications having the same biological function, in particular encoding proteins with the same or substantially the same biological function. They may be naturally occurring variations, such as sequences from other plant varieties or species, or mutations. These mutations may occur naturally or may be obtained by mutagenesis techniques. The allelic variations may be naturally occurring allelic variants as well as synthetically produced or genetically engineered variants. Structurally equivalents can, for example, be identified by testing the binding of said polypeptide to antibodies or computer based predictions. Structurally equivalents have the similar immunological characteristic, e.g. comprise similar epitopes.

By “hybridizing” it is meant that such nucleic acid molecules hybridize under conventional hybridization conditions, preferably under stringent conditions such as described by, e.g., Sambrook (“Molecular Cloning; A Laboratory Manual”, 2nd edition, Cold Spring Harbor Laboratory Press, Cold Spring Harbor, NY (1989)) or in “Current Protocols in Molecular Biology”, John Wiley & Sons, N.Y. (1989), chapter 6.3.1-6.3.6.)

According to the invention, DNA as well as RNA molecules of the corresponding nucleic acid molecules of the invention can be used as probes. Further, as template for the identification of functional homologues Northern blot assays as well as Southern blot assays can be performed. The Northern blot assay advantageously provides further information about the expressed gene product: e.g. expression pattern, occurrence of processing steps, like splicing and capping, etc. The Southern blot assay provides additional information about the chromosomal localization and organization of the gene being related to the nucleic acid molecule of the invention.

A preferred, non-limiting example of stringent hybridization conditions are hybridizations in 6× sodium chloride/sodium citrate (=SSC) at approximately 45° C., followed by one or more wash steps in 0.2×SSC, 0.1% SDS at 50 to 65° C., for example at 50° C., 55° C. or 60° C. The skilled worker knows that these hybridization conditions differ as a function of the type of the nucleic acid and, for example when organic solvents are present, with regard to the temperature and concentration of the buffer. The temperature under “standard hybridization conditions” differs for example as a function of the type of the nucleic acid between 42° C. and 65° C., preferably between 45° C. and 50° C. in an aqueous buffer with a concentration of 0.1×, 0.5×, 1×, 2×, 3×, 4×or 5×SSC (pH 7.2). If organic solvent(s) is/are present in the above-mentioned buffer, for example 50% formamide, the temperature under standard conditions is approximately 40° C., 42° C. or 45° C. The hybridization conditions for DNA:DNA hybrids are preferably for example 0.1×SSC and 20° C., 25° C., 30° C., 35° C., 40° C. or 45° C., preferably between 30° C. and 45° C. The hybridization conditions for DNA:RNA hybrids are preferably for example 0.1×SSC and 30° C., 35° C., 40° C., 45° C., 50° C. or 55° C., preferably between 45° C. and 55° C. The above-mentioned hybridization temperatures are determined for example for a nucleic acid approximately 100 by (=base pairs) in length and a G+C content of 50% in the absence of formamide. The skilled worker knows to determine the hybridization conditions required with the aid of textbooks, for example the ones mentioned above, or from the following textbooks: Sambrook et al., “Molecular Cloning”, Cold Spring Harbor Laboratory, 1989; Hames and Higgins (ed.), “Nucleic Acids Hybridization: A Practical Approach”, IRL Press at Oxford University Press, Oxford, 1985; Brown (ed.), “Essential Molecular Biology: A Practical Approach”, IRL Press at Oxford University Press, Oxford, 1991.

A further example of one such stringent hybridization condition is hybridization at 4×SSC at 65° C., followed by a washing in 0.1×SSC at 65° C. for one hour. Alternatively, an exemplary stringent hybridization condition is in 50% formamide, 4×SSC at 42° C. Further, the conditions during the wash step can be selected from the range of conditions delimited by low-stringency conditions (approximately 2×SSC at 50° C.) and high-stringency conditions (approximately 0.2×SSC at 50° C., preferably at 65° C.) (20×SSC : 0.3 M sodium citrate, 3 M NaCl, pH 7.0). In addition, the temperature during the wash step can be raised from low-stringency conditions at room temperature, approximately 22° C., to higher-stringency conditions at approximately 65° C. Both of the parameters salt concentration and temperature can be varied simultaneously, or else one of the two parameters can be kept constant while only the other is varied. Denaturants, for example formamide or SDS, may also be employed during the hybridization. In the presence of 50% formamide, hybridization is preferably effected at 42° C. Relevant factors like 1) length of treatment, 2) salt conditions, 3) detergent conditions, 4) competitor DNAs, 5) temperature and 6) probe selection can be combined case by case so that not all possibilities can be mentioned herein.

Thus, in a preferred embodiment, Northern blots are prehybridized with RothiHybri-Quick buffer (Roth, Karlsruhe) at 68° C. for 2 h. Hybridization with radioactive labeled probe is done overnight at 68° C. Subsequent washing steps are performed at 68° C. with 1×SSC. For Southern blot assays the membrane is prehybridized with Rothi-Hybri-Quick buffer (Roth, Karlsruhe) at 68° C. for 2 h. The hybridization with radioactive labeled probe is conducted over night at 68° C. Subsequently the hybridization buffer is discarded and the filter shortly washed using 2×SSC; 0.1% SDS. After discarding the washing buffer new 2×SSC; 0.1% SDS buffer is added and incubated at 68° C. for 15 minutes. This washing step is performed twice followed by an additional washing step using 1×SSC; 0.1% SDS at 68° C. for 10 min.

Some examples of conditions for DNA hybridization (Southern blot assays) and wash step are shown herein below:

(1) Hybridization conditions can be selected, for example, from the following conditions:
(a) 4×SSC at 65° C.,
(b) 6×SSC at 45° C.,
(c) 6×SSC, 100 mg/ml denatured fragmented fish sperm DNA at 68° C.,
(d) 6×SSC, 0.5% SDS, 100 mg/ml denatured salmon sperm DNA at 68° C.,
(e) 6×SSC, 0.5% SDS, 100 mg/ml denatured fragmented salmon sperm DNA, 50% formamide at 42° C.,
(f) 50% formamide, 4×SSC at 42° C.,
(g) 50% (v/v) formamide, 0.1% bovine serum albumin, 0.1% Ficoll, 0.1% polyvinylpyrrolidone, 50 mM sodium phosphate buffer pH 6.5, 750 mM NaCl, 75 mM sodium citrate at 42° C.,
(h) 2× or 4×SSC at 50° C. (low-stringency condition), or
(i) 30 to 40% formamide, 2 x or 4×SSC at 42° C. (low-stringency condition).
   (2) Wash steps can be selected, for example, from the following conditions:
(a) 0.015 M NaCl/0.0015 M sodium citrate/0.1% SDS at 50° C.,
(b) 0.1×SSC at 65° C.,
(c) 0.1×SSC, 0.5% SDS at 68° C.,
(d) 0.1×SSC, 0.5% SDS, 50% formamide at 42° C.,
(e) 0.2×SSC, 0.1% SDS at 42° C.,
(f) 2×SSC at 65° C. (low-stringency condition),

Polypeptides having above-mentioned activity, i.e. conferring the production or the increased production of the fine chemical alpha-tocopherol, or gamma-tocopherol as compared to a corresponding, e.g. non-transformed, wild type non-human organism, like a microorganism or a plant cell, plant or part thereof, can be encoded by other DNA sequences which hybridize to the sequences shown in the respective line in Table I, application no. 7, columns 5 and 8, preferably the coding region thereof, at least under relaxed hybridization conditions and which encode the expression of polypeptides conferring the production or the increased production of the respective fine chemical alpha-tocopherol, or gamma-tocopherol as compared to a corresponding, e.g. non-transformed, wild type non-human organism, like a microorganism or a plant cell, plant or part thereof.

Further, some applications have to be performed at low stringency hybridization conditions, without any consequences for the specificity of the hybridization. For example, a Southern blot analysis of total DNA could be probed with a nucleic acid molecule of the present invention and washed at low stringency (55° C. in 2×SSPE, 0,1% SDS). The hybridization analysis could reveal a simple pattern of only genes encoding polypeptides of the present invention or used in the process of the invention, e.g. having the herein-mentioned activity of conferring the production or the increased production of the fine chemical as compared to a corresponding, e.g. non-transformed, wild type non-human organism, like a microorganism or a plant cell, plant or part thereof. A further example of such low-stringent hybridization conditions is 4×SSC at 50° C. or hybridization with 30 to 40% formamide at 42° C. Such molecules comprise those which are fragments, analogues or derivatives of the polypeptide of the invention or used in the process of the invention and differ, for example, by way of amino acid and/or nucleotide deletion(s), insertion(s), substitution (s), addition(s) and/or recombination (s) or any other modification(s) known in the art either alone or in combination from the above-described amino acid sequences or their underlying nucleotide sequence(s). However, it is preferred to use high stringency hybridization conditions.

Hybridization should advantageously be carried out with fragments of at least 5, 10, 15, 20, 25, 30, 35 or 40 bp, advantageously at least 50, 60, 70 or 80 bp, preferably at least 90, 100 or 110 bp. Most preferably are fragments of at least 15, 20, 25 or 30 bp. Preferably are also hybridizations with at least 100 by or 200 bp, very especially preferably at least 400 by in length. In an especially preferred embodiment, the hybridization should be carried out with the entire nucleic acid sequence with conditions described above.

The terms “fragment”, “fragment of a sequence” or “part of a sequence” mean a truncated sequence of the original sequence referred to. The truncated sequence (nucleic acid or protein sequence) can vary widely in length; the minimum size being a sequence of sufficient size to provide a sequence with at least a comparable function and/or activity of the original sequence or molecule referred to, or hybridizing with the nucleic acid molecule of the invention or used in the process of the invention under stringent conditions, while the maximum size is not critical. In some applications, the maximum size usually is not substantially greater than that required to provide the desired activity and/or function(s) of the original sequence.

Typically, the truncated amino acid sequence or molecule will range from about 5 to about 310 amino acids in length. More typically, however, the sequence will be a maximum of about 250 amino acids in length, preferably a maximum of about 200 or 100 amino acids. It is usually desirable to select sequences of at least about 10, 12 or 15 amino acids, up to a maximum of about 20 or 25 amino acids.

The term “epitope” relates to specific immunoreactive sites within an antigen, also known as antigenic determinates. These epitopes can be a linear array of monomers in a polymeric composition—such as amino acids in a protein—or consist of or comprise a more complex secondary or tertiary structure. Those of skill will recognize that immunogens (i.e. substances capable of eliciting an immune response) are antigens; however, some antigen, such as haptens, are not immunogens but may be made immunogenic by coupling to a carrier molecule. The term “antigen” includes references to a substance to which an antibody can be generated and/or to which the antibody is specifically immunoreactive.

In an embodiment the present invention relates to an epitope of the polypeptide of the present invention or used in the process of the present invention and conferring the production or the increased production of the fine chemical as compared to a corresponding, e.g. non-transformed, wild type non-human organism, like a microorganism or a plant cell, plant or part thereof.

The term “one or several amino acids” relates to at least one amino acid but not more than that number of amino acids, which would result in a homology of below 50% identity. Preferably, the identity is more than 70%, 80%, 85%, 90%, 91%, 92%, 93%, 94%, 95%, 96%, 97%, 98%, 99% or 99.5% identity.

Further, the nucleic acid molecule of the invention comprises a nucleic acid molecule, which is a complement of one of the nucleotide sequences of above-mentioned nucleic acid molecules or a portion thereof. A nucleic acid molecule or its sequence which is complementary to one of the nucleotide molecules or sequences shown in the respective line in Table I, application no. 7, columns 5 or 8, preferably the coding region thereof, homologs or fragments thereof, is one which is sufficiently complementary to one of the nucleotide molecules or sequences shown in the respective line in Table I, application no. 7, columns 5 or 8, preferably the coding region thereof, homologs or fragments thereof, such that it can hybridize to one of the nucleotide sequences shown in Table I, application no. 7, columns 5 or 8, preferably the coding region thereof, homologs or fragments thereof, thereby forming a stable duplex. Preferably, the hybridization is performed under stringent hybridization conditions. However, a complement of one of the herein disclosed sequences is preferably a sequence complement thereto according to the base pairing of nucleic acid molecules well known to the skilled person. For example, the bases A and G undergo base pairing with the bases T and U or C, resp. and visa versa. Modifications of the bases can influence the base-pairing partner.

The nucleic acid molecule of the invention comprises a nucleotide sequence which is at least about 30%, 35%, 40%, 45%, 50%, 55%, 60%, 65%, 70%, 75%, 80%, 90%, 95%, 96%, 97%, 98%, 99%, 99.5% or more homologous to a nucleotide sequence shown in Table I, application no. 7, columns 5 or 8, preferably the coding region thereof, homologs or fragments thereof, and preferably has above-mentioned activity, in particular conferring the production or the increased production of the fine chemical alpha-tocopherol, or gamma-tocopherol, respectively, after increasing the activity or an activity of a gene as shown in the respective line in Table I or of a gene product, e.g. as shown in the respective line in Table II, application no. 7, column 5 or 8, by for example in one embodiment expression either in the cytosol or in an organelle such as a plastid or mitochondria or both, preferably in a plastid, or in another embodiment non-targeted or targeted.

The nucleic acid molecule of the invention comprises a nucleotide sequence or molecule which hybridizes, preferably hybridizes under stringent conditions as defined herein, to one of the nucleic acid molecule shown in the respective line in Table I, preferably Table IB, application no. 7, columns 5 or 8, preferably the coding region thereof, fragments or homologs thereof, and encodes a protein having above-mentioned activity, e.g. conferring the production or the increased production of the respective fine chemical alpha-tocopherol, or gamma-tocopherol as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted, and optionally, the activity thereof is selected from the group consisting of 3-deoxy-7-phosphoheptulonate synthase, 3-deoxy-D-arabinoheptulosonate-7-phosphatesynthase, ABC transporter permease subunit, acetyltransferase, acid shock protein, adenylate kinase, adenylosuccinate lyase, amino-acid acetyltransferase, ankyrin repeat family protein, At1g29350-protein, ATP-binding component of a transport system, b1163-protein, b1219-protein, b1234-protein, b1522-protein, b1921-protein, b2360-protein, b2548-protein, b2613-protein, b2673-protein, b2812-protein, b3890-protein, b3989-protein, b4029-protein, b4050-protein, b4056-protein, calcium-dependent protein kinase, carbohydrate kinase, colanic acid biosynthesis protein, coproporphyrinogen oxidase, cyclin D , DNA polymerase subunit, electron transfer flavoprotein subunit beta, geranylgeranyl pyrophosphate synthase, geranyltranstransferase, gluconate transport system permease 3, glucose dehydrogenase, glucose-6-phosphate 1-dehydrogenase, glutaredoxin, glycerol dehydrogenase, glycerol-3-phosphate dehydrogenase, glycolate utilization protein, harpin-induced family protein, homocitrate synthase, hydrolase, isopropylmalate isomerase large subunit, lipopolysaccharide biosynthesis protein, MADS box protein transcription factor, major facilitator superfamily transporter protein, malate dehydrogenase, monothiol glutaredoxin, monthiol glutaredoxin, multiple antibiotic resistance protein, murein transglycosylase, N-(5′-phosphoribosyl) anthranilate isomerase, NAD-dependent epimerase/dehydratase, NADP-dependent malic enzyme, O-antigen chain length determinant, phosphatidylserine decarboxylase, phospho-2-dehydro-3-deoxyheptonate aldolase, phosphoadenosine phosphosulfate reductase, phosphofructokinase, photosystem II protein, polygalacturonase, polyphosphate kinase, precorrin methylase, protein kinase, protein phosphatase, putative transport system permease protein, pyruvate kinase, RNA-binding protein, Sec-independent protein translocase subunit, serine protease, sll1761-protein, slr1917-protein, small nuclear ribonucleo-protein , ThiF family protein, thioredoxin, thioredoxin family protein, threonine synthase, transcription factor, transcriptional regulator, transport protein, trehalose-phosphatase, TTC1386-protein, ycl049c-protein, ydl235c-protein, ydr183w-protein, yer106w-protein, ylr178c-protein, ypr170c-protein, and zinc finger protein, respectively.

Moreover, the nucleic acid molecule of the invention can comprise only a portion of the coding region of one of the nucleic acid molecules depicted in Table I, application no. 7, columns 5 or 8, for example a fragment which can be used as a probe or primer or a fragment encoding a biologically active portion of the polypeptide of the present invention or of a polypeptide used in the process of the present invention, i.e. having above-mentioned activity, e.g. conferring the production or the increased production of the respective fine chemical alpha-tocopherol, or gamma-tocopherol as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted. The nucleotide sequences determined from the cloning of the present protein-according-to-the-invention-encoding gene allows for the generation of probes and primers designed for use in identifying and/or cloning its homologues from other cell types and organisms. The probe/primer typically comprises a substantially purified oligonucleotide. The oligonucleotide typically comprises a region of nucleotide sequence that hybridizes under stringent conditions to at least about 12, 15 preferably about 20 or 25, more preferably about 40, 50 or 75 consecutive nucleotides of a sense strand of one of the sequences set forth, e.g., in the respective line in Table I, application no. 7, column 5 or 8, preferably the coding region thereof, or a homolog or a fragment thereof, an anti-sense sequence of one of the sequences, e.g., set forth in the respective line in Table I, application no. 7, columns 5 or 8, preferably the coding region thereof, or a homolog or a fragment thereof or naturally occurring mutants thereof. Primers based on a nucleic acid molecule of invention can be used in PCR reactions to clone homologs of the polypeptide of the invention or of the polypeptide used in the process of the invention, e.g. as the primers described in the examples of the present invention, e.g. as shown in the examples. A PCR with the primers shown in the respective line in Table III, column 8 will result in a fragment of the gene product as shown in Table II, application no. 7, column 5.

Primer sets are interchangeable. The person skilled in the art knows how to design and combine said primers to result in the desired product, e.g. in a full length clone or a partial sequence. Probes based on the sequences of the nucleic acid molecule of the invention or used in the process of the present invention can be used to detect transcripts or genomic sequences encoding the same or homologous proteins. The probe can further comprise a label group attached thereto, e.g. the label group can be a radioisotope, a fluorescent compound, an enzyme, or an enzyme co-factor. Such probes can be used as a part of a genomic marker test kit for identifying cells which express a polypeptide of the invention or used in the process of the present invention, such as by measuring a level of an encoding nucleic acid molecule in a sample of cells, e.g. detecting mRNA levels or determining, whether a genomic gene comprising the sequence of the polynucleotide of the invention or used in the processes of the present invention has been mutated or deleted.

As the expression of a respective nucleic acids of the invention is related to the synthesis of the respective fine chemical alpha-tocopherol, or gamma-tocopherol its function as a probe extends to the detection of microorganisms, plant tissues, plants, plant variets, plant ecotypes or plant genera with varying capability or potential for synthesis of the respective fine chemical alpha-tocopherol, or gamma-tocopherol. Therefore in one embodiment the present invention relates to a method for analyzing the capability or potential of a plant tissue, a plant, a plant variety or plant ecotype to produce the fine chemical alpha-tocopherol, or gamma-tocopherol by using the nucleic acid of the invention or parts thereof as a probe to detect the amount of the nucleic acid of the invention in the non-human organism or a part thereof in comparison to another non-human organism.

The nucleic acid molecule of the invention encodes a polypeptide or portion thereof which includes an amino acid sequence which is sufficiently homologous to the amino acid sequence shown in respective line in Table II, application no. 7, columns 5 or 8 such that the protein or portion thereof maintains the ability to confer the production or the increased production of the respective fine chemical alpha-tocopherol, or gamma-tocopherol as compared to a corresponding, e.g. non-transformed, wild type non-human organism, like a microorganism or a plant cell, plant or part thereof, in particular increasing the activity as mentioned above or as described in the examples in microorganisms or plants.

As used herein, the language “sufficiently homologous” refers to proteins or portions thereof which have amino acid sequences which include a minimum number of identical or equivalent amino acids (e.g. an amino acid which has a similar side chain as an amino acid in one of the sequences of the polypeptide of the present invention) to an amino acid sequence shown in the respective line in Table II, application no. 7, columns 5 or 8 such that the protein or portion thereof is able to confer the production or the increased production of the respective fine chemical alpha-tocopherol, or gamma-tocopherol as compared to a corresponding, e.g. non-transformed, wild type non-human organism, like a microorganism or a plant cell, plant or part thereof. For example having the activity of a protein as shown in the respective line in Table II, application no. 7, column 3 and as described herein.

In an embodiment of the present invention, the nucleic acid molecule of the present invention comprises a nucleic acid molecule that encodes a portion of the protein of the present invention. The protein is at least about 30%, 35%, 40%, 45% 50%, 55%, 60%, 65%, 70%, 75%, 80%, 85%, 90%, 91%, 92%, 93%, 94%, 95%, 96%, 97%, 98%, 99%, 99.5% or more homologous to an entire amino acid sequence shown in the respective line of Table II, application no. 7, column 5 or 8 and has the above-mentioned activity, e.g. conferring the production or the increased production of the respective fine chemical alpha-tocopherol, or gamma-tocopherol as compared to a corresponding, e.g. non-transformed, wild type non-human organism, like a microorganism or a plant cell, plant or part thereof by, for example in a embodiment expression either in the cytosol or in an organelle such as a plastid or mitochondria or both, preferably in a plastid, or in another embodiment by targeted or non-targeted expression.

Portions of proteins encoded by the nucleic acid molecule of the invention show preferably the above-mentioned activity, e.g. conferring the production or the increased production of the fine chemical as compared to a corresponding, e.g. non-transformed, wild type non-human organism, like a microorganism or a plant cell, plant or part thereof.

As mentioned herein, the term “biologically active portion” is intended to include a portion, e.g., a domain/motif, that confers the production or the increased production of the fine chemical as compared to a corresponding, e.g. non-transformed, wild type non-human organism, like a microorganism or a plant cell, plant or part thereof or has an immunological activity such that it binds to an antibody binding specifically to the polypeptide of the present invention or a polypeptide used in the process of the present invention for conferring the production or the increased production of the fine chemical as compared to a corresponding, e.g. non-transformed, wild type non-human organism, like a microorganism or a plant cell, plant or part thereof.

The invention further relates to nucleic acid molecules or methods using said nucleic acid molecules that differ from one of the nucleotide sequences shown in the respective line in Table I, application no. 7, columns 5 or 8, preferably the coding region thereof, or fragments or homologs thereof, due to degeneracy of the genetic code but encode a polypeptide of the present invention, in particular a polypeptide having the above-mentioned activity, e.g. as those polypeptides depicted in the respective line in Table II, application no. 7, columns 5 or 8, fragments or homologs thereof. Advantageously, the nucleic acid molecule of the invention comprises, or in an other embodiment has, a nucleotide sequence encoding a protein comprising, or in an other embodiment having, an amino acid sequence shown in the respective line in Table II, application no. 7, columns 5 or 8, fragments or homologs thereof. In a further embodiment, the nucleic acid molecule of the invention encodes a full length protein which is substantially homologous to an amino acid sequence shown in the respective line in Table II, application no. 7, columns 5 or 8, or homologs thereof. However, in an embodiment, the nucleic acid molecule of the present invention does not consist of the sequence shown in the respective line in Table I, preferably Table IA, application no. 7, columns 5 or 8, preferably the coding region thereof, but the functional homologs thereof.

In addition, it will be appreciated by those skilled in the art that DNA sequence polymorphisms that lead to changes in the amino acid sequences may exist within a population of the non-human organism of the present invention. Also these non-human organisms are encompassed by the respective non-human organism according to the invention.

As used herein, the terms “gene” and “recombinant gene” refer to nucleic acid molecules comprising an open reading frame encoding the polypeptide of the invention or comprising the nucleic acid molecule of the invention or encoding the polypeptide used in the process of the present invention, preferably from a plant, especially a crop plant, or from a microorganism useful for the method of the invention. Such genetic polymorphism in the gene encoding the polypeptide of the invention or comprising the nucleic acid molecule of the invention may exist among individuals within a population due to natural variation. Such natural variations can typically result in 1 to 5% variance in the nucleotide sequence of the gene. Any and all such nucleotide variations and resulting amino acid polymorphisms in genes encoding a polypeptide of the invention or comprising a the nucleic acid molecule of the invention that are the result of natural variation and that do not alter the functional activity as described are intended to be within the scope of the invention.

Nucleic acid molecules corresponding to natural variants of a nucleic acid molecule of the invention, which can also be a cDNA, can be isolated based on their homology to the nucleic acid molecules disclosed herein using the nucleic acid molecule of the invention, or a portion thereof, as a hybridization probe according to standard hybridization techniques under stringent hybridization conditions.

Accordingly, in another embodiment, a nucleic acid molecule of the invention is at least 15, 20, 25 or 30 nucleotides in length. Preferably, it hybridizes under stringent conditions to a nucleic acid molecule comprising a nucleotide sequence of the nucleic acid molecule of the present invention or used in the process of the present invention, e.g. comprising the sequence shown in the respective line in Table I, application no. 7, columns 5 or 8, preferably the coding region thereof. In another embodiment the nucleic acid molecule is preferably at least 20, 30, 50, 100, 250 or more nucleotides in length.

The term “hybridizes under stringent conditions” is defined above. In an embodiment, the term “hybridizes under stringent conditions” is intended to describe conditions for hybridization and washing under which nucleotide sequences at least 30%, 40%, 50% or 65% identical to each other typically remain hybridized to each other. Preferably, the conditions are such that sequences at least about 70%, 75%, 80%, 85%, 90% or 95% or more identical to each other typically remain hybridized to each other.

Preferably, a nucleic acid molecule of the invention that hybridizes under stringent conditions to a sequence shown in the respective line in Table I, application no. 7, columns 5 or 8, preferably the coding region thereof, corresponds to a naturally-occurring nucleic acid molecule of the invention. As used herein, a “naturally-occurring” nucleic acid molecule refers to a RNA or DNA molecule having a nucleotide sequence that occurs in nature (e.g. encodes a natural protein). Preferably, the nucleic acid molecule encodes a natural protein having above-mentioned activity, e.g. conferring the production or the increased production of the respective fine chemical alpha-tocopherol, or gamma-tocopherol as compared to a corresponding, e.g. non-transformed, wild type non-human organism, like a microorganism or a plant cell, plant or part thereof after increasing the expression or activity thereof or the activity of a protein of the invention or used in the process of the invention, in an embodiment for example expression either in the cytosol or in an organelle such as a plastid or mitochondria, preferably in plastids, or, in another embodiment by targeted or non-targeted expression.

In addition to naturally-occurring variants of the sequences of the polypeptide or nucleic acid molecule of the invention as well as of the polypeptide or nucleic acid molecule used in the process of the invention that may exist in the population, the skilled artisan will further appreciate that changes can be introduced by mutation into a nucleotide sequence of the nucleic acid molecule of the invention, e.g. encoding the polypeptide of the invention or used in the process of the present invention, thereby leading to changes in the amino acid sequence of the encoded said polypeptide, without altering the functional ability of the polypeptide, preferably not decreasing said activity, preferably increasing said activity.

For example, nucleotide substitutions leading to amino acid substitutions at “non-essential” amino acid residues can be made in a sequence of the nucleic acid molecule of the invention or used in the process of the invention, e.g. shown in the respective line in Table I, application no. 7, columns 5 or 8, preferably the coding region thereof, or fragments or homologs thereof.

With regard to the activity of a polypeptide a “non-essential” amino acid residue is a residue that can be altered without altering the activity of said polypeptide, whereas an “essential” amino acid residue is required for an activity as mentioned above, e.g. conferring the production or the increased production of the fine chemical as compared to a corresponding, e.g. non-transformed, wild type non-human organism, like a microorganism or a plant cell, plant or part thereof. Other amino acid residues, however, (e.g., those that are not conserved or only semi-conserved in the domain having said activity) may not be essential for activity and thus are likely to be amenable to alteration without altering said activity.

Further, a person skilled in the art knows that the codon usage between organisms can differ. In the enclosed sequence protocol according to WIPO ST 25 the respective donor organism codon usage “translation” is used.The person skilled in the art refore, he may adapt the codon usage in the nucleic acid molecule of the present invention to the usage of the organism or the cell compartment for example of the plastid or mitochondria in which the polynucleotide or polypeptide is expressed.

Accordingly, the invention relates to nucleic acid molecules encoding a polypeptide having above-mentioned activity, in a non-human organisms or parts thereof by for example expression either in the cytosol or in an organelle such as a plastid or mitochondria or both, preferably in plastids, that contain changes in amino acid residues that are not essential for said activity. Such polypeptides differ in amino acid sequence from a sequence depicted in the respective line in Table II, especially Table IIA, application no. 7, columns 5 or 8, but retain said activity described herein. The nucleic acid molecule can comprise a nucleotide sequence encoding a polypeptide, wherein the polypeptide comprises an amino acid sequence at least about 50% identical to an amino acid sequence shown in the respective line in Table II, application no. 7, columns 5 or 8, and is capable of conferring the production or the increased production of the respective fine chemical alpha-tocopherol, or gamma-tocopherol as compared to a corresponding, e.g. non-transformed, wild type non-human organism, like a microorganism or a plant cell, plant or part thereof, after increasing its activity for example in an embodiment by expression either in the cytosol or in an organelle such as a plastid or mitochondria or both, preferably in plastids, or, in another embodiment by targeted or non-targeted expression. Preferably, the protein encoded by the nucleic acid molecule is at least about 60%, 70%, 80%, 90%, 95%, 96%, 97%, 98%, 99% or 99.5% identical to the sequence shown in the respective line in Table II, application no. 7, columns 5 or 8.

To determine the-percentage identity (=homology, herein used interchangeably) of two amino acid sequences or of two nucleic acid molecules, the sequences are written one underneath the other for an optimal comparison (for example gaps may be inserted into the sequence of a protein or of a nucleic acid in order to generate an optimal alignment with the other protein or the other nucleic acid). The amino acids or nucleobases at the respective corresponding positions are then compared. If a position in one sequence is occupied by the same amino acid or the same nucleobase as the corresponding position in the other sequence, the molecules are homologous at this position. The-percentage identity between the two sequences is a function of the number of identical positions shared by the sequences (i.e. % homology=identity=number of identical positions/total number of positions×100).

For the determination of the-percentage identity (=homology) of two or more amino acid sequences or of two or more nucleic acid molecules several computer software programs have been developed. The homology of two or more sequences can be calculated with for example the software Fasta, which presently has been used in the version Fasta 3 (Pearson W. R. and Lipman D. J., PNAS 85, 2444 (1988); Pearson W. R., Methods in Enzymology 183, 63 (1990)). Another useful program for the calculation of homologies of different sequences is the standard blast program, which is included in the Biomax PEDANTTM software (Biomax, Munich, Federal Republic of Germany). This leads unfortunately sometimes to suboptimal results since blast does not always include complete sequences of the subject and the query. Nevertheless as this program is very efficient it can be used for the comparison of a huge number of sequences. The following settings are typically used for such a comparison of sequences: -p Program Name [String]; -d Database [String]; default =nr; -i Query File [File In]; default=stdin; -e Expectation value (E) [Real]; default=10.0; -m alignment view options: 0 =pairwise; 1=query-anchored showing identities; 2=query-anchored no identities; 3=flat query-anchored, show identities; 4=flat query-anchored, no identities; 5=query-anchored no identities and blunt ends; 6=flat query-anchored, no identities and blunt ends; 7=XML Blast output; 8=tabular; 9 tabular with comment lines [Integer]; default=0; -o BLAST report Output File [File Out] Optional; default=stdout; -F Filter query sequence (DUST with blastn, SEG with others) [String]; default=T; -G Cost to open a gap (zero invokes default behavior) [Integer]; default=0; -E Cost to extend a gap (zero invokes default behavior) [Integer]; default=0; -X X dropoff value for gapped alignment (in bits) (zero invokes default behavior); blastn 30, megablast 20, tblastx 0, all others 15 [Integer]; default=0; -I Show GI's in deflines [T/F]; default=F; -q Penalty for a nucleotide mismatch (blastn only) [Integer]; default=-3; -r Reward for a nucleotide match (blastn only) [Integer]; default=1; -v Number of database sequences to show one-line descriptions for (V) [Integer]; default=500; -b Number of database sequence to show alignments for (B) [Integer]; default=250; -f Threshold for extending hits, default if zero; blastp 11, blastn 0, blastx 12, tblastn 13; tblastx 13, megablast 0 [Integer]; default=0; -g Perfom gapped alignment (not available with tblastx) [T/F]; default=T; -Q Query Genetic code to use [Integer]; default=1; -D DB Genetic code (for tblast[nx] only) [Integer]; default=1; -a Number of processors to use [Integer]; default=1; -O SeqAlign file [File Out] Optional; -J Believe the query defline [T/F]; default=F; -M Matrix [String]; default=BLOSUM62; -W Word size, default if zero (blastn 11, megablast 28, all others 3) [Integer]; default=0; -z Effective length of the database (use zero for the real size) [Real]; default=0; -K Number of best hits from a region to keep (off by default, if used a value of 100 is recommended) [Integer]; default=0; -P 0 for multiple hit, 1 for single hit [Integer]; default=0; -Y Effective length of the search space (use zero for the real size) [Real]; default=0; -S Query strands to search against database (for blast[nx], and tblastx); 3 is both, 1 is top, 2 is bottom [Integer]; default=3; -T Produce HTML output [T/F]; default=F; -I Restrict search of database to list of GI's [String] Optional; -U Use lower case filtering of FASTA sequence [T/F] Optional; default=F; -y X dropoff value for ungapped extensions in bits (0.0 invokes default behavior); blastn 20, megablast 10, all others 7 [Real]; default =0.0; -Z X dropoff value for final gapped alignment in bits (0.0 invokes default behavior); blastn/megablast 50, tblastx 0, all others 25 [Integer]; default=0; -R PSI-TBLASTN checkpoint file [File In] Optional; -n MegaBlast search [T/F]; default=F; -L Location on query sequence [String] Optional; -A Multiple Hits window size, default if zero (blastn/megablast 0, all others 40 [Integer]; default=0; -w Frame shift penalty (00F algorithm for blastx) [Integer]; default=0; -t Length of the largest intron allowed in tblastn for linking HSPs (0 disables linking) [Integer]; default=0.

Results of high quality are reached by using the algorithm of Needleman and Wunsch or Smith and Waterman. Therefore programs based on said algorithms are preferred. Advantageously the comparisons of sequences can be done with the program PileUp (J. Mol. Evolution., 25, 351 (1987), Higgins et al., CABIOS 5, 151 (1989)) or preferably with the programs “Gap” and “Needle”, which are both based on the algorithms of Needleman and Wunsch (J. Mol. Biol. 48, 443 (1970)), and “BestFit”, which is based on the algorithm of Smith and Waterman (Adv. Appl. Math. 2, 482 (1981)). “Gap” and “BestFit” are part of the GCG softwarepackage (Genetics Computer Group, 575 Science Drive, Madison, Wis., USA 53711 (1991); Altschul et al., (Nucleic Acids Res. 25, 3389 (1997)), “Needle” is part of the The European Molecular Biology Open Software Suite (EMBOSS) (Trends in Genetics 16 (6), 276 (2000)). Therefore preferably the calculations to determine the-percentages of sequence homology are done with the programs “Gap” or “Needle” over the whole range of the sequences. The following standard adjustments for the comparison of nucleic acid sequences were used for “Needle”: matrix: EDNAFULL, Gap_penalty: 10.0, Extend_penalty: 0.5. The following standard adjustments for the comparison of nucleic acid sequences were used for “Gap”: gap weight: 50, length weight: 3, average match: 10.000, average mismatch: 0.000.

For example a sequence, which has 80% homology with sequence SEQ ID NO: 1815 at the nucleic acid level is understood as meaning a sequence which, upon comparison with the sequence SEQ ID NO: 1815 by the above-mentioned program “Needle” with the above-mentioned parameter set, has a 80% homology.

Homology between two polypeptides is understood as meaning the identity of the amino acid sequence over in each case the entire sequence length which is calculated by comparison with the aid of the above program “Needle” using Matrix: EBLOSUM62, Gap_penalty: 8.0, Extend_penalty: 2.0.

For example a sequence which has a 80% homology with sequence SEQ ID NO: 1816 at the protein level is understood as meaning a sequence which, upon comparison with the sequence SEQ ID NO: 1816 by the above-mentioned program “Needle” with the above-mentioned parameter set, has a 80% homology.

Functional equivalents of the nucleic acid molecules according to the present invention and/or used in the process according to the present invention represent an embodiment of homologs. Functional equivalents derived from the nucleic acid molecules as shown in the respective line in Table I, application no. 7, columns 5 or 8, preferably the coding region thereof, according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the nucleic acid molecules as shown in the respective line in Table I, application no. 7, columns 5 or 8, preferably the coding region thereof, according to the invention and encode polypeptides having essentially the same properties as the polypeptide as shown in the respective line in Table II, application no. 7, columns 5 or 8.

Functional equivalents derived from one of the polypeptides as shown in the respective line in Table II, application no. 7, columns 5 or 8 according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the polypeptides as shown in the respective line in Table II, application no. 7, columns 5 or 8 according to the invention and having essentially the same properties as the polypeptide as shown in the respective line in Table II, application no. 7, columns 5 or 8.

“Essentially the same properties” of a functional equivalent is above all understood as meaning that the functional equivalent has the above-mentioned activity, by for example in an embodiment expression either in the cytosol or in an organelle such as a plastid or mitochondria or both, or, in another embodiment by targeted or non-targeted expression, while increasing the amount of protein, activity or function of said functional equivalent in an organism, e.g. a microorganism, a plant or plant tissue or animal tissue, plant or animal cells or a part of the same.

A nucleic acid molecule encoding a homolog to a polypeptide depicted in the respective line in Table II, application no. 7, columns 5 or 8 can be created by introducing one or more nucleotide substitutions, additions or deletions into the nucleic acid molecule of the present invention, in particular of Table I, application no. 7, columns 5 or 8, in the respective line, such that one or more amino acid substitutions, additions or deletions are introduced into the encoded protein. Mutations can be introduced into the nucleic acid molecules as depicted in the respective line in Table I, application no. 7, columns 5 or 8, preferably the coding region thereof, by standard techniques, such as site-directed mutagenesis, PCR-mediated mutagenesis or other methods known to the person skilled in the art.

Preferably, conservative amino acid substitutions are made at one or more predicted non-essential amino acid. A “conservative amino acid substitution” is one in which the amino acid is replaced with an amino acid having a similar side chain. Families of amino acids having similar side chains have been defined in the art. These families include amino acids with basic side chains (e.g. lysine, arginine, histidine), acidic side chains (e.g. aspartic acid, glutamic acid), uncharged polar side chains (e.g. glycine, asparagine, glutamine, serine, threonine, tyrosine, cysteine), nonpolar side chains (e.g. alanine, valine, leucine, isoleucine, proline, phenylalanine, methionine, tryptophane), beta-branched side chains (e.g. threonine, valine, isoleucine) and aromatic side chains (e.g. tyrosine, phenylalanine, tryptophane, histidine).

Thus, a predicted non-essential amino acid in a polypeptide of the invention or a polypeptide used in the process of the invention is preferably replaced with another amino acid from the same family. Alternatively, in another embodiment, mutations can be introduced randomly along all or part of a coding sequence of a nucleic acid molecule of the invention or used in the process of the invention, such as by saturation mutagenesis, and the resultant mutants can be screened for activity described herein to identify mutants that retain or even have increased activity, e.g. conferring the production or the increased production of the fine chemical as compared to a corresponding, e.g. non-transformed, wild type non-human organism, like a microorganism or a plant cell, plant or part thereof.

Following mutagenesis of one of the sequences as shown herein, the encoded protein can be expressed recombinantly and the activity of the protein can be determined using, for example, assays described herein (see Examples) or by measuring the resulting fine chemical production in an organism expressing the mutagenized form of the sequence in comparison to an organism expressing the original non-mutagenized form of the sequence.

Homologues of the nucleic acid sequences used, with the sequences shown in the respective line in Table I, application no. 7, columns 5 or 8, preferably the coding region thereof, comprise also allelic variants with at least approximately 30%, 35%, 40%, 45%, 50%, 55%, 60%, 65%, 70%, 75%, 80%, 85%, 90%, 91%, 92%, 93%, 94% 95%. 96%, 97%, 98%, 99%, 99.5% or more % homology with one of the nucleotide sequences shown or the above-mentioned derived nucleic acid sequences or their homologues or derivatives or parts of these.

Allelic variants encompass in particular functional variants which can be obtained by deletion, insertion or substitution of nucleotides from the sequences shown in the respective line, preferably in Table I, columns 5 or 8, preferably the coding region thereof, or from the derived nucleic acid sequences, the intention being, however, that the enzymatic activity or the biological activity of the resulting proteins synthesized is advantageously retained or increased.

In one embodiment of the present invention, the nucleic acid molecule of the invention or used in the process of the invention comprises the sequences shown in the respective line in any of the Table I, application no. 7, columns 5 or 8, preferably the coding region thereof, fragments or homologs thereof. It is preferred that the nucleic acid molecule comprises as little as possible other nucleotides not shown in the respective line in any one of Table I, application no. 7, columns 5 or 8, preferably the coding region thereof, fragments or homologs thereof. In one embodiment, the nucleic acid molecule comprises less than 500, 400, 300, 200, 100, 90, 80, 70, 60, 50 or 40 further nucleotides. In a further embodiment, the nucleic acid molecule comprises less than 30, 20 or 10 further nucleotides. In one embodiment, the nucleic acid molecule used in the process of the invention is identical to the sequences shown in the respective line in Table I, application no. 7, columns 5 or 8, preferably the coding region thereof.

Also preferred is that the nucleic acid molecule used in the process of the invention encodes a polypeptide comprising the sequence shown in the respective line in Table II, application no. 7, columns 5 or 8, fragments or homologs thereof. In one embodiment, the nucleic acid molecule encodes less than 150, 130, 100, 80, 60, 50, 40 or 30 further amino acids. In a further embodiment, the encoded polypeptide comprises less than 20, 15, 10, 9, 8, 7, 6 or 5 further amino acids. In one embodiment used in the inventive process, the encoded polypeptide is identical to the sequences shown in the respective line in Table II, application no. 7, columns 5 or 8.

In one embodiment, the nucleic acid molecule of the invention or used in the process encodes a polypeptide comprising the sequence shown in the respective line in Table II, application no. 7, columns 5 or 8, or fragments or homologs thereof, and comprises less than 100 further nucleotides. In a further embodiment, said nucleic acid molecule comprises less than 30 further nucleotides. In one embodiment, the nucleic acid molecule used in the process is identical to a coding sequence of the sequences shown in the respective line in Table I, application no. 7, columns 5 or 8.

Polypeptides (=proteins), which still have the essential biological or enzymatic activity of the polypeptide of the present invention conferring the production or the increased production of the respective fine chemical alpha-tocopherol, or gamma-tocopherol as compared to a corresponding, e.g. non-transformed, wild type non-human organism, like a microorganism or a plant cell, plant or part thereof, i.e. whose activity is essentially not reduced, are polypeptides with at least 10%, 20%, 30%, 40%, 50%, 60%, 70%, 80%, 90%, 95% or more of the wild type biological activity or enzymatic activity, advantageously, the activity is essentially not reduced in comparison with the activity of a polypeptide shown in the respective line in Table II, application no. 7, columns 5 or 8 expressed under identical conditions.

Homologues of nucleic acid molecules shown in the respective line in Table I, application no. 7, columns 5 or 8, preferably the coding region thereof, or fragments thereof, or of the derived sequences shown in the respective line in Table II, application no. 7, columns 5 or 8, or fragments thereof, also mean truncated sequences, cDNA, single-stranded DNA or RNA of the coding and noncoding DNA sequence. Homologues of said sequences are also understood as meaning derivatives, which comprise noncoding regions such as, for example,

UTRs, introns, terminators, enhancers or promoter variants. The promoters upstream of the nucleotide sequences stated can be modified by one or more nucleotide substitution(s), insertion(s) and/or deletion(s) without, however, interfering with the functionality or activity either of the promoters, the open reading frame (=ORF) or with the 3′-regulatory region such as terminators or other 3′-regulatory regions, which are far away from the ORF. It is furthermore possible that the activity of the promoters is increased by modification of their sequence, or that they are replaced completely by more active promoters, even promoters from heterologous organisms. Appropriate promoters are known to the person skilled in the art and are mentioned herein below.

Accordingly, one embodiment of the invention relates to a host cell, which has been transformed stably or transiently with the vector according to the invention or the nucleic acid molecule according to the invention or the nucleic acid construct or the expression construct according to the invention.

Depending on the non-human host organism, the non-human organisms used in the process according to the invention are cultured or grown in a manner with which the skilled worker is familiar.

As a rule, microorganisms are grown in a liquid medium comprising a carbon source, usually in the form of sugars, a nitrogen source, usually in the form of organic nitrogen sources such as yeast extract or salts such as ammonium sulfate, trace elements such as iron salts, manganese salts, magnesium salts, and, if appropriate, vitamins, at temperatures between 0° C. and 100° C., preferably between 10° C. and 60° C., while passing in oxygen. In the event the microorganism is anaerobe, no oxygen is blown through the culture medium. The pH value of the liquid nutrient medium may be kept constant, that is to say regulated during the culturing phase, or not. The organisms may be cultured batch wise, semi batch wise or continuously. Nutrients may be provided at the beginning of the fermentation or fed in semi continuously or continuously. Advantageously microorganisms such as algae are grown under sunlight in open ponds or in fermentors illuminated with a light intensity between 10 to 2000 μmol×m⁻²×s⁻¹, preferred between 100 to 1000 μmol×m⁻²×s⁻¹, more preferred between 200 to 800 μmol×m⁻²×s⁻¹, most preferred between 300 to 600 μmol×m⁻²×⁻¹. The cells are grown between several hours for example 3 to 48 h and several days 1 to 20 days, preferably 2 to 10 days. Algae as autotrophic organisms grow well in the presence of light as energy source, inorganic hydrogen donors and CO₂ as sole carbon source.

As a rule, plants used in the process according to the invention are grown according to general knowledge of a skilled worker.

The fine chemical produced can be isolated from the non-human organism by methods with which the skilled worker is familiar. For example via extraction, salt precipitation or chromatography, like ion-exchange chromatography. To this end, the non-human organisms, especially the microorganism or the plant, may be disrupted beforehand. The process according to the invention can be conducted batch wise, semi batch wise or continuously. A summary of known culture and isolation techniques can be found in the textbook by Chmiel (“Bioprozeβtechnik 1, Einführung in die Bioverfahrenstechnik”, Gustav Fischer Verlag, Stuttgart, 1991)), Demain et al. (“Industrial Microbiology and Biotechnology”, 2ndedition, ASM Press, Washington, D.C., 1999, ISBN 1-55581-128-0) or in the textbook by Storhas (“Bioreaktoren und periphere Einrichtungen”, Vieweg Verlag, Braunschweig/Wiesbaden, 1994)).

In one embodiment, the present invention relates to a polypeptide encoded by the nucleic acid molecule according to the present invention, preferably conferring the production of or an increase in the fine chemical content in an non-human organism or cell after generating or increasing the expression or activity of said nucleic acid molecule, either in the cytosol or in an organelles such as a plastid or mitochondrion or both, preferably in a plastid, or in another embodiment by targeted or non-targeted expression.

The present invention also relates to a process for the production of a polypeptide according to the present invention, the polypeptide being expressed in a host cell according to the invention, preferably in a microorganism or a plant cell, plant or a part thereof, especially in a transgenic microorganism or a transgenic plant cell, plant or a part thereof.

In an embodiment, the nucleic acid molecule used in the process for the production of the polypeptide (FCRP) is derived from a microorganism such as a eukaryotic or prokaryotic microorganism, preferably from a eukaryotic microorganism, such as an algae or Saccharomyces cerevisiae; in an embodiment thereof this polypeptide is produced in another microorganism or in another embodiment thereof this polypeptide is produced in a plant cell, plant or a part thereof. In another embodiment, the nucleic acid molecule used in the process for the production of the polypeptide is derived from a plant, in an embodiment thereof the polypeptide is produced in a microorganism or in another embodiment thereof this polypeptide is produced in another plant, a part or cell thereof.

The skilled worker knows that protein and DNA expressed in different non-human organisms differ in many respects and properties, e. g. DNA modulation and imprinting, such as methylation or post-translational modification, as for example glucosylation, phosphorylation, acetylation, myristoylation, ADP-ribosylation, farnesylation, carboxylation, sulfation, ubiquination, etc. though having the same coding sequence. Preferably, the cellular expression control of the corresponding protein differs accordingly in the control mechanisms controlling the activity and expression of an endogenous protein or another prokaryotic or eukaryotic protein. One major difference between proteins expressed in prokaryotic or eukaryotic organisms is the amount and pattern of glycosylation. For example in E. coli there are no glycosylated proteins. Proteins expressed in yeasts have a high mannose content in the glycosylated proteins, whereas in plants the glycosylation pattern is complex.

The polypeptide (FCRP) of the present invention is preferably produced by recombinant DNA techniques. For example, a nucleic acid molecule encoding the protein is cloned into a vector (as described above), the vector is introduced into a host cell (as described above) and said polypeptide is expressed in the host cell. Said polypeptide (FCRP) can then be isolated from the cells by an appropriate purification scheme using standard protein purification techniques. Alternative to recombinant expression, the polypeptide or peptide of the present invention can be synthesized chemically using standard peptide synthesis techniques.

Moreover, a native polypeptide conferring the increase of the respective fine chemical alpha-tocopherol, or gamma-tocopherol in a non-human organism or a part thereof can be isolated from cells (e.g., endothelial cells), for example using the antibody of the present invention as described below, in particular, an antibody against proteins having 3-deoxy-7-phosphoheptulonate synthase, 3-deoxy-D-arabinoheptulosonate-7-phosphatesynthase, ABC transporter permease subunit, acetyltransferase, acid shock protein, adenylate kinase, adenylosuccinate lyase, amino-acid acetyltransferase, ankyrin repeat family protein, At1g29350-protein, ATP-binding component of a transport system, b1163-protein, b1219-protein, b1234-protein, b1522-protein, b1921-protein, b2360-protein, b2548-protein, b2613-protein, b2673-protein, b2812-protein, b3890-protein, b3989-protein, b4029-protein, b4050-protein, b4056-protein, calcium-dependent protein kinase, carbohydrate kinase, colanic acid biosynthesis protein, coproporphyrinogen oxidase, cyclin D , DNA polymerase subunit, electron transfer flavoprotein subunit beta, geranylgeranyl pyrophosphate synthase, geranyltranstransferase, gluconate transport system permease 3, glucose dehydrogenase, glucose-6-phosphate 1-dehydrogenase, glutaredoxin, glycerol dehydrogenase, glycerol-3-phosphate dehydrogenase, glycolate utilization protein, harpin-induced family protein, homocitrate synthase, hydrolase, isopropylmalate isomerase large subunit, lipopolysaccharide biosynthesis protein, MADS box protein transcription factor, major facilitator superfamily transporter protein, malate dehydrogenase, monothiol glutaredoxin, monthiol glutaredoxin, multiple antibiotic resistance protein, murein transglycosylase, N-(5′-phosphoribosyl) anthranilate isomerase, NAD-dependent epimerase/dehydratase, NADP-dependent malic enzyme, O-antigen chain length determinant, phosphatidylserine decarboxylase, phospho-2-dehydro-3-deoxyheptonate aldolase, phosphoadenosine phosphosulfate reductase , phosphofructokinase, photosystem II protein, polygalacturonase, polyphosphate kinase, precorrin methylase, protein kinase, protein phosphatase, putative transport system permease protein, pyruvate kinase, RNA-binding protein, Sec-independent protein translocase subunit, serine protease, sll1761-protein, slr1917-protein, small nuclear ribonucleoprotein , ThiF family protein, thioredoxin, thioredoxin family protein, threonine synthase, transcription factor, transcriptional regulator, transport protein, trehalose-phosphatase, TTC1386-protein, ycl049c-protein, ydl235c-protein, ydr183w-protein, yer106w-protein, ylr178c-protein, ypr170c-protein, or zinc finger protein activity, respectively, or an antibody against polypeptides as shown in the respective line in Table II, application no. 7, columns 5 or 8, or fragments or homologs thereof which can be produced by standard techniques utilizing the polypeptid of the present invention or fragment thereof, i.e., the polypeptide of this invention (FCRP). Preferred are monoclonal antibodies.

In an embodiment, the present invention relates to a polypeptide encoded by a nucleic acid molecule of the invention or obtainable by a process of the invention. Said polypeptide confers preferably the aforementioned activity, in particular, the polypeptide confers the generation or the increased production of the fine chemical in a cell or an organism or a part thereof after generating or increasing the cellular activity, e.g. by generating or increasing the expression or the specific activity of the polypeptide.

In one embodiment, the present invention relates to a polypeptide as depicted in the respective line in Table II, especially in Table IIA or especially in Table IIB, application no. 7, columns 5 or 8, or fragment or homolog thereof or as coded by the nucleic acid molecule shown in the respective line in Table I, especially Table IA or especially Table IB, application no. 7, columns 5 or 8, preferably the coding region thereof, or fragment or homolog thereof.

In an embodiment, the present invention relates to a polypeptide comprising or consisting of a consensus sequence shown in the respective line in Table IV, application no. 7, column 8 or at least one polypeptide motif shown in the respective line in Table IV, application no. 7, column 8.

In an embodiment thereof, the present invention relates to a polypeptide comprising or consisting of a consensus sequence shown in the respective line in Table IV, application no. 7, column 8 or comprising at least one polypeptide motif shown in the respective line in Table IV, application no. 7, column 8, whereby not more than 20, 15, 10, 9, 8, 7, 6, 5, 4, 3, 2, or 1, or 0 of the amino acids can be replaced by any amino acid.

In another embodiment thereof, the present invention relates to a polypeptide comprising or consisting of a consensus sequence shown in the respective line in Table IV, application no. 7, column 8 or comprising at least one polypeptide motif shown in the respective line in Table IV, application no. 7, column 8, whereby not more than 15%, 10%, 5%, 4%, 3%, 2% or1% or 0% of the amino acids defined as distinct specific amino acids are/is replaced by another amino acid.

In another embodiment thereof, the present invention relates to a polypeptide comprising or consisting of a consensus sequence shown in the respective line in Table IV, application no. 7, column 8 or comprising at least one polypeptide motif shown in the respective line in Table IV, application no. 7, column 8, whereby not more than 20, 15, 10, 9, 8, 7, 6, 5, 4, 3, 2, or 1, or 0 amino acids are inserted into the consensus sequence or polypeptide motif.

In an embodiment of the present invention, the polypeptide of the present invention comprises or consists of a consensus sequence or at least one polypeptide motif shown in the respective line in Table IV, application no. 7, column 8, whereby said polypeptide distinguishes over the sequence depicted in the respective line in Table II, application no. 7, columns 5 or 8 by one or more amino acids. In one embodiment, said polypeptide of the invention distinguishes over the sequence shown in the respective line in Table II, application no. 7, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 7, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in the respective line in Table II, application no. 7, columns 5 and 8.

In an embodiment, the invention relates to a polypeptide conferring an increase in the respective fine chemical alpha-tocopherol, or gamma-tocopherol in a non-human organism, especially a microorganism or a plant, or a part thereof, being encoded by the nucleic acid molecule of the invention or used in the process of the invention and having a sequence which distinguishes over the sequence as shown in the respective line in Table II, application no. 7, columns 5 or 8 by one or more amino acids (but not exceeding 5%, preferably 4%, 3%, 2%, 1%, 0.5%, 0.2% or 0.1%). In an embodiment, said polypeptide of the invention does not comprise or consist of the sequence shown in the respective line in Table II, application no. 7, columns 5 or 8. In an embodiment, said polypeptide of the present invention is less than 100%, 99.999%, 99.99%, 99.9% or 99% identical. In one embodiment, said polypeptide which differs at least in one or more amino acids (but not exceeding 5%, preferably 4%, 3%, 2%, 1%, 0.5%, 0.2% or 0.1%) from the polypeptide shown in the respective line in Table II, application no. 7, columns 5 and 8 does not comprise a protein of the sequence shown in the respective line in Table II A and/or II B, application no. 7, columns 5 or 8.

In an embodiment, the present invention relates to a polypeptide having the activity of the protein as shown in the respective line in Table II, application no. 7, column 3, which distinguishes over the sequence depicted in the respective line in Table II, application no. 7, columns 5 or 8 by one or more amino acids, preferably by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 7, columns 5 and 8 by not more than 80%, 70%, 60%, 50%, 40%, 30%, 20%, 10%, 5%, 4%, 3%, 2%, 1% or 0.5%.

In another embodiment the polypeptide of the invention takes the form of a preprotein consisting of a plastidial or mitochondrial transit peptide joint to a polypeptide having the activity of the protein as shown in Table II, column 3, from which the transit peptide is preferably cleaved off upon transport of the preprotein into the organelle for example into the plastid or mitochondrion.

In another embodiment the polypeptide of the invention takes not the form of a preprotein.

The terms “protein” and “polypeptide” used in this application are interchangeable. “Polypeptide” refers to a polymer of amino acids (amino acid sequence) and does not refer to a specific length of the molecule. Thus peptides and oligopeptides are included within the definition of polypeptide. This term does also refer to or include post-translational modifications of the polypeptide, for example, glycosylations, acetylations, phosphorylations and the like. Included within the definition are, for example, polypeptides containing one or more analogs of an amino acid (including, for example, unnatural amino acids, etc.), polypeptides with substituted linkages, as well as other modifications known in the art, both naturally occurring and nonnaturally occurring.

Another aspect of the present invention pertains to isolated FCRP, or biologically active portions thereof. An “isolated” or “purified” protein or biologically active portion thereof is substantially free of cellular material when produced by recombinant DNA techniques or chemical precursors or other chemicals when chemically synthesized.

The language “substantially free of cellular material” includes preparations of the polypeptide of the invention in which the protein is separated from cellular components of the cells in which it is naturally or recombinantly produced. In an embodiment, the language “substantially free of cellular material” includes preparations having less than about 30% (by dry weight) of “contaminating protein”, more preferably less than about 20% of “contaminating protein”, still more preferably less than about 10% of “contaminating protein”, and most preferably less than about 5% “contaminating protein”. The term “contaminating protein” relates to polypeptides, which differ from the polypeptide in question of the present invention. When the polypeptide of the present invention or biologically active portion thereof is recombinantly produced, it is also preferably substantially free of culture medium, i.e., culture medium represents less than about 20%, more preferably less than about 10%, and most preferably less than about 5% of the volume of the protein preparation. The language “substantially free of chemical precursors or other chemicals” includes preparations in which the polypeptide of the present invention is separated from chemical precursors or other chemicals, which are involved in the synthesis of the protein. The language “substantially free of chemical precursors or other chemicals” includes preparations having less than about 30% (by dry weight) of chemical precursors, other chemicals or other proteins, which differ from the polypeptide in question. Other chemical precursors, other chemicals or other proteins, which are not identical to the proteins as shown in the respective line in Table II, column 5 or 8, or fragments or homologs thereof, are all collectively named as impurities. The term “chemical precursors” shall mean in the sense of the specification chemical substances, which are intermediates of the biochemical pathway within the organism or within the cell(s) of the non-human organism for example glucose-6-phoshat, citrate, fumarate, homoserine etc. The term “other chemicals” shall mean in the sense of the specification chemical substances, which are end products of the biochemical pathway within the organism or within the cell(s) of the organism for example amino acids such as lysine, alanine etc; fatty acids such as linolenic acid, eicosapantaenoic acid etc, sugars such as glucose, mannose, ribose, desoxy ribose etc, vitamins such as vitamin C, vitamin B2 etc. and all other chemical substances of the cell. The term “other proteins” shall mean in the sense of the specification all other proteins, which are not identical to the proteins mentioned in the respective line in Table II, columns 5 or 8, or fragments or homologs thereof. The fine chemical preparations advantageously shall have less than about 25% impurities, preferably less than about 20% impurities, still more preferably less than about 10% impurities, and most preferably less than about 5% impurities. In preferred embodiments, isolated proteins or biologically active portions thereof lack contaminating proteins from the same organism from which the polypeptide of the present invention is derived. Typically, such proteins are produced by recombinant techniques.

A polypeptide of the invention can participate in the process of the present invention. The polypeptide or a portion thereof comprises preferably an amino acid sequence, which is sufficiently homologous to an amino acid sequence shown in the respective line in Table II A and/or II B, application no. 7, columns 5 or 8.

Further, the polypeptide can have an amino acid sequence which is encoded by a nucleotide sequence which hybridizes, preferably hybridizes under stringent conditions as described above, to a nucleotide sequence of the nucleic acid molecule of the present invention. Accordingly, the polypeptide has an amino acid sequence which is encoded by a nucleotide sequence that is at least about 35%, 40%, 45%, 50%, 55%, 60%, 65%, 70%, 75%, 80%, 85%, 90%, 91%, 92%, 93%, 94%, 95%, 96%, 97%, 98%, 99%, 99.5% or more homologous to one of the amino acid sequences as shown in the respective line in Table II A and/or II B, application no. 7, columns 5 or 8, or fragments thereof. The preferred polypeptide of the present invention preferably possesses at least one of the activities according to the invention and described herein. A preferred polypeptide of the present invention includes an amino acid sequence encoded by a nucleotide sequence which hybridizes, preferably hybridizes under stringent conditions, to a nucleotide sequence shown in the respective line in Table I A and/or I B, application no. 7, columns 5 or 8, preferably the coding region thereof, or fragments thereto or which is homologous thereto, as defined above.

The polypeptide of the present invention can vary from the sequences shown in the respective line in Table II A and/or II B, application no. 7, columns 5 or 8 in amino acid sequence due to natural variation or mutagenesis, as described in detail herein. Accordingly, the polypeptide comprise an amino acid sequence which is at least about 35%, 40%, 45%, 50%, 55%, 60%, 65%, 70%, 75%, 80%, 85% or 90, 91%, 92%, 93%, 94%, 95%, 96%, 97%, 98%, 99%, 99.5% or more homologous to an entire amino acid sequence shown in the respective line in Table II A and/or II B, application no. 7, columns 5 or 8.

Biologically active portions of a polypeptide of the present invention include peptides comprising amino acid sequences derived from the amino acid sequence of the polypeptide of the present invention or used in the process of the present invention, e.g., the amino acid sequence shown in the respective line in Table II, application no. 7, columns 5 or 8 or the amino acid sequence of a protein homologous thereto, which include fewer amino acids than a full length polypeptide of the present invention or used in the process of the present invention or the full length protein which is homologous to a polypeptide of the present invention or used in the process of the present invention depicted herein, and exhibit at least one activity of a polypeptide of the present invention or used in the process of the present invention.

Typically, biologically (or immunologically) active portions of i.e. peptides are, for example, 5, 10, 15, 20, 30, 35, 36, 37, 38, 39, 40, 50, 75, 100, 125, 150 or more amino acids in length; especially they comprise a domain or motif with at least one activity or epitope of a polypeptide of the present invention or used in the process of the present invention. Moreover, other biologically active portions, in which other regions of the polypeptide are deleted, can be prepared by recombinant techniques and evaluated for one or more of the activities described herein.

Manipulation of the nucleic acid molecule of the invention may result in the production of a protein having differences from the protein as shown in the respective line in Table II, application no. 7, column 5 or 8. Differences shall mean at least one amino acid different from the sequences as shown in the respective line in Table II, application no. 7, column 5 or 8, preferably at least 2, 3, 4, 5, 6, 7, 8, 9 or 10 amino acids (especially but not exceeding 0.1%, preferably 0.2%, 0.5%, 1%, 2%, 3% 4% or 5%), more preferably at least 15, 20, 25, 30, 35, 40, 45 or 50 amino acids (especially but not exceeding 0.5%, preferably 1%, 2%, 3% 4%, 5%, 10% or 25%) different from the sequences as shown in the respective line in Table II, application no. 7, column 5 or 8. These proteins may be improved in efficiency or activity, or may be more stable and therefore present in greater numbers in the cell as compared to the wild-type cell.

Any mutagenesis strategies for the polypeptide of the present invention or the polypeptide used in the process of the present invention to result in increasing said activity are not meant to be limiting; variations on these strategies will be readily apparent to one skilled in the art. Using such strategies, and incorporating the mechanisms disclosed herein, the nucleic acid molecule and polypeptide of the invention may be utilized to generate algae, ciliates, fungi, other microorganisms like C. glutamicum or plants or parts thereof, expressing proteins as shown in the respective line in Table II, application no. 7, column 5 or 8, or mutated proteins thereof. The nucleic acid molecules and polypeptide molecules of the invention are expressed such that the yield, production, and/or efficiency of production of a desired compound is improved.

This desired compound may be any natural product of the respective non-human organism, especially a microorganism or a plant, which includes the final products of biosynthesis pathways and intermediates of naturally-occurring metabolic pathways, as well as molecules which do not naturally occur in the metabolism of said cells of said non-human organism, but which are produced by said cells of the invention.

The invention also provides chimeric or fusion proteins.

As used herein, a “chimeric protein” or “fusion protein” comprises a polypeptide of the present invention operatively linked to a polypeptide whereby the latter does not confer on its own above-mentioned activity, in particular, which does not confer an increase of content of the fine chemical in a cell or a non-human organism or a part thereof, if its activity is increased.

In one embodiment, a protein (=polypeptide) as shown in the respective line in Table II, application no. 7, column 5 or 8 refers to a polypeptide having an amino acid sequence corresponding to the polypeptide of the invention, especially to a polypeptide as shown in Table II, application no. 7, columns 5 or 8 or homologs thereof, or used in the process of the invention, whereas a “non- inventive protein or polypeptide” or “other polypeptide” refers to a polypeptide having an amino acid sequence corresponding to a protein which is not substantially homologous to a polypeptide of the invention, preferably which is not substantially homologous to a polypeptide or protein as shown in the respective line in Table II, application no. 7, column 5 or 8 or homologs thereof, e.g., a protein which does not confer the activity described herein and which is derived from the same or a different organism.

Within the fusion protein, the term “operatively linked” is intended to indicate that the polypeptide of the invention or a polypeptide used in the process of the invention and the “other polypeptide” or a part thereof are fused to each other so that both sequences fulfill the proposed function addicted to the sequence used. The “other polypeptide” can be fused to the N-terminus or C-terminus preferable to the C-terminus of the polypeptide of the invention or used in the process of the invention. For example, in one embodiment the fusion protein is a GST-LMRP fusion protein in which the sequences of the polypeptide of the invention or the polypeptide used in the process of the invention are fused to the C-terminus of the GST sequences. Such fusion proteins can facilitate the purification of recombinant polypeptides of the invention or a polypeptide useful in the process of the invention.

In another preferred embodiment, the fusion protein is a polypeptide of the invention or a polypeptide used in the process of the invention containing a heterologous signal sequence, preferably at its N-terminus. In certain host cells (e.g., mammalian host cells), expression and/or secretion of a polypeptide of the invention or a polypeptide used in the process of the invention can be increased through use of a heterologous signal sequence. As already mentioned above, targeting sequences, are required for targeting the gene product into specific cell compartment (for a review, see Kermode, Crit. Rev. Plant Sci. 15 (4), 285 (1996) and references cited therein), for example into the vacuole, the nucleus, all types of plastids, such as amyloplasts, chloroplasts, chromoplasts, the extracellular space, the mitochondria, the endoplasmic reticulum, elaioplasts, peroxisomes, glycosomes, and other compartments of cells or extracellular. Sequences, which must be mentioned in this context, are, in particular, the signal-peptide- or transit-peptide-sequences which are known by the person skilled in the art. For example, plastid or mitochondrial-transit-peptide- sequences enable the targeting of the expression product into the plastids or the mitochondria, respectively, of a plant cell or the mitochondria of a microorganism. Targeting sequences are especially known for eukaryotic and to a lower extent for prokaryotic organisms and can advantageously be operable linked with the nucleic acid molecule of the present invention to achieve an expression in one of said compartments or extracellular.

Preferably, a chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. The fusion gene can be synthesized by conventional techniques including automated DNA synthesizers. Alternatively, PCR amplification of gene fragments can be carried out using anchor primers, which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons (1992)). Moreover, many expression vectors are commercially available that already encode a fusion moiety (e.g., a GST polypeptide). The nucleic acid molecule of the invention can be cloned into such an expression vector such that the fusion moiety is linked in-frame to the encoded protein.

Furthermore, folding simulations and computer redesign of structural motifs of the protein of the invention can be performed using appropriate computer programs (Olszewski, Proteins 25, 286 (1996); Hoffman, Comput. Appl. Biosci. 11, 675 (1995)). Computer modeling of protein folding can be used for the conformational and energetic analysis of detailed peptide and protein models (Monge, J. Mol. Biol. 247, 995 (1995); Renouf, Adv. Exp. Med. Biol. 376, 37 (1995)). The appropriate programs can be used for the identification of interactive sites the polypeptide of the invention or polypeptides used in the process of the invention and its substrates or binding factors or other interacting proteins by computer assistant searches for complementary peptide sequences (Fassina, Immunomethods 114 (1994)). This can be used to identify motifs and domains of the polypeptides according to the present invention or used in the process of the present invention. Further appropriate computer systems for the design of protein and peptides are described in the prior art, for example in Berry, Biochem. Soc. Trans. 22, 1033 (1994); Wodak, Ann. N.Y. Acad. Sci. 501, 1 (1987); Pabo, Biochemistry 25, 5987 (1986). The results obtained from the above-described computer analysis can be used for, e.g., the preparation of peptidomimetics of the protein of the invention or fragments thereof. Such pseudopeptide analogues of the natural amino acid sequence of the protein may very efficiently mimic the parent protein (Benkirane, J. Biol. Chem. 271, 33218 (1996)). For example, the replacement of Gly-Gly in a protein of the present invention or a fragment thereof by the easily available achiral omega-amino acid delta-amino valeric acid results in the substitution of —CH₂CONHCH₂— by CH₂CH₂CH₂CH₂—, thereby providing a replacement of an amido group by an ethylen group, and a convenient strategy for constructing a peptidomimetic (Banerjee, Biopolymers 39, 769 (1996)).

Furthermore, a three-dimensional and/or crystallographic structure of the protein of the invention or used in the process of the present invention and the identification of motifs and/or interactive sites of the polypeptide of the invention or used in the process of the present invention and its substrates or binding factors can be used for the design of mutants with modulated binding or turnover activities. For example, the active center of the polypeptide of the present invention can be modeled and amino acids participating in the catalytic reaction can be modulated to increase or decrease the binding of the substrate to activate or improve the polypeptide. The identification of the active center, respective motifs and the amino acids involved in the catalytic reaction facilitates the screening for mutants having an increased activity.

Another embodiment of the invention relates to an antibody, which binds specifically to the polypeptide according to the invention or a portion thereof, i.e. specific fragments or epitopes of such a polypeptide.

The antibodies of the invention can be used to identify and isolate the polypeptide according to the invention in any organism, preferably microorganism or plants. These antibodies can be monoclonal antibodies, polyclonal antibodies or synthetic antibodies as well as fragments of antibodies, such as Fab, Fv or scFv fragments etc. Antibodies can be made by many well-known methods (see, e.g. Harlow and Lane, “Antibodies; A Laboratory Manual”, Cold Spring Harbor Laboratory, Cold Spring Harbor, N.Y., (1988)). Briefly, purified antigen can be injected into an animal in an amount and in intervals sufficient to elicit an immune response.

Antibodies can either be purified directly, or spleen cells can be obtained from the animal. The cells can then be fused with an immortal cell line and screened for antibody secretion. The antibodies can be used to screen nucleic acid clone libraries for cells secreting the antigen. Those positive clones can then be sequenced. See, for example, Kelly et al., Bio/Technology 10, 163 (1992); Bebbington et al., Bio/Technology 10, 169 (1992).

As the nucleic acid molecules of the invention and/or the expression thereof is related to the synthesis of the respective fine chemical alpha-tocopherol, or gamma-tocopherol its function as a probe extends to the detection of microorganisms, plant tissues, plants, plant variets, plant ecotypes or plant genera with varying, advantageously increased, capability or potential for synthesis of the respective fine chemical alpha-tocopherol, or gamma-tocopherol.

Therefore in one embodiment the present invention relates to a method for analyzing the capability or potential of a plant tissue, a plant, a plant variety or ecotype to produce the respective fine chemical alpha-tocopherol, or gamma-tocopherol by using the respective antibody of the invention as a probe to detect the amount of the polypeptide encoded by said nucleic acid molecule of the invention in a non-human organism in comparison to another organism.

Therefore in one embodiment the invention relates to a method to probe a non-human organism for its capability for the production of the respective fine chemical comprising the steps of
a) analyzing the sequence and/or the expression of the respective nucleic acid molecule of the invention in least two non-human organisms which differ in the capability to produce the respective fine chemical;
b) associate the different sequence and/or expression level of the nucleic acid molecules of the invention with their different capability to produce the respective fine chemical;
c) analyze the sequence and/or the expression of the nucleic acid molecule of the invention in at least a third non-human organism;
d) predict the capability of the at least third organism to produce the respective fine chemical based by comparing the sequence and/or expression of the nucleic acid of the invention to the sequences and/or expression levels of the nucleic acid molecule of the invention of the at least two organisms analyzed in step a.

The phrases “selectively binds” and “specifically binds” with the polypeptide refer to a binding reaction that is determinative of the presence of the polypeptide in a heterogeneous population of polypeptides and other biologics. Thus, under designated immunoassay conditions, the specified antibodies bound to a particular polypeptide do not bind in a significant amount to other polypeptides present in the sample. Selective binding of an antibody under such conditions may require an antibody that is selected for its specificity for a particular polypeptide. A variety of immunoassay formats may be used to select antibodies that selectively bind with a particular polypeptide. For example, solid-phase ELISA immunoassays are routinely used to select antibodies selectively immunoreactive with a polypeptide. See Harlow and Lane, “Antibodies, A Laboratory Manual,” Cold Spring Harbor Publications, New York, (1988), for a description of immunoassay formats and conditions that could be used to determine selective binding.

In some instances, it is desirable to prepare monoclonal antibodies from various hosts. A description of techniques for preparing such monoclonal antibodies may be found in Stites et al., eds., “Basic and Clinical Immunology,” (Lange Medical Publications, Los Altos, Calif., Fourth Edition) and references cited therein, and in Harlow and Lane, “Antibodies, A Laboratory Manual,” Cold Spring Harbor Publications, New York (1988).

Gene expression in non-human organism, like microorganism or plants, especially plants, is regulated by the interaction of protein transcription factors with specific nucleotide sequences within the regulatory region of a gene. One example of transcription factors are polypeptides that contain zinc finger (ZF) motifs. Each ZF module is approximately 30 amino acids long and folds around a zinc ion. The DNA recognition domain of a ZF protein is an α-helical structure that inserts into the major groove of the DNA double helix. The module contains three amino acids that bind to the DNA with each amino acid contacting a single base pair in the target DNA sequence. ZF motifs are arranged in a modular repeating fashion to form a set of fingers that recognize a contiguous DNA sequence. For example, a three-fingered ZF motif will recognize 9 bp of DNA. Hundreds of proteins have been shown to contain ZF motifs with between 2 and 37 ZF modules in each protein (Isalan M. et al., Biochemistry 37 (35), 12026 (1998); Moore M. et al., Proc. Natl. Acad. Sci. USA 98 (4), 1432 (2001) and Moore M. et al., Proc. Natl. Acad. Sci. USA 98 (4), 1437 (2001); U.S. Pat. No. 6,007,988 and U.S. Pat. No. 6,013,453).

The regulatory region of a gene contains many short DNA sequences (cis-acting elements) that serve as recognition domains for transcription factors, including ZF proteins. Similar recognition domains in different genes allow the coordinate expression of several genes encoding enzymes in a metabolic pathway by common transcription factors. Variation in the recognition domains among members of a gene family facilitates differences in gene expression within the same gene family, for example, among tissues and stages of development and in response to environmental conditions.

Typical ZF proteins contain not only a DNA recognition domain but also a functional domain that enables the ZF protein to activate or repress transcription of a specific gene. Experimentally, an activation domain has been used to activate transcription of the target gene (U.S. Pat. No. 5,789,538 and patent application WO 95/19431), but it is also possible to link a transcription repressor domain to the ZF and thereby inhibit transcription (WO 00/47754 and WO 01/002019). It has been reported that an enzymatic function such as nucleic acid cleavage can be linked to the ZF (WO 00/20622).

The invention provides a method that allows one skilled in the art to isolate the regulatory region of one or more FCRP encoding genes from the genome of a non-human organism, preferably a plant cell and to design zinc finger transcription factors linked to a functional domain that will interact with the regulatory region of the gene. The interaction of the zinc finger protein with the gene can be designed in such a manner as to alter expression of the gene and preferably thereby to confer the production or the increased production of the fine chemical as compared to a corresponding, e.g. non-transformed, wild type non-human organism, like a microorganism or a plant cell, plant or part thereof.

A further embodiment of the invention also relates to a method for the generation of a transgenic host or host cell, e.g. a eukaryotic or prokaryotic cell, preferably a transgenic microorganism, a transgenic plant cell or a transgenic plant tissue or a transgenic plant, which comprises introducing, into the plant cell, the plant or a part thereof , the nucleic acid molecule according to the invention, the nucleic acid construct according to the invention or the vector according to the invention or the expression cassette according to the invention.

A further embodiment of the invention also relates to a method for the stable generation of a host or host cell, prokaryotic or eukaryotic cell, preferably a transgenic microorganism such as a transgenic algae, a transgenic plant cell or a transgenic plant or a transgenic part thereof, which comprises introducing, into the plant cell, the plant or a part thereof, the nucleic acid construct according to the invention, the vector according to the invention, the expression cassette according to the invention, or the nucleic acid molecule according to the invention, whereby the introduction of the nucleic acid molecule, nucleic acid construct, expression cassette and/or vector is in such manner that the transformants are stable during the propagation of the host in respect of the introduced nucleic acid molecules, nucleic acid construct, expression cassette and/or vector.

A further embodiment of the invention also relates to a method for the transient generation of a host or host cell, prokaryotic or eukaryotic cell, preferably a transgenic microorganism such as a transgenic algae, a transgenic plant cell or a transgenic plant or a transgenic part thereof, which comprises introducing, into the plant cell, the plant or a part thereof, the nucleic acid construct according to the invention, the vector according to the invention, the expression cassette according to the invention or the nucleic acid molecule according to the invention, whereby the introduced nucleic acid molecule, nucleic acid construct, expression cassette and/or vector is not integrated into the genome of the host or host cell. Therefore the transformants are not stable during the propagation of the host in respect of the introduced nucleic acid molecules, nucleic acid construct, expressions cassette and/or vector.

In the process according to the invention, transgenic non-human organisms are also to be understood as meaning—if they take the form of plants—plant cells, plant tissues, plant organs such as root, shoot, stem, seed, flower, tuber or leaf, or intact plants which are grown for the production of the fine chemical.

Growing is to be understood as meaning for example culturing the transgenic plant cells, plant tissue or plant organs on or in a nutrient medium or the intact plant or part thereof on or in a substrate, for example in hydroponic culture, potting compost or on a field soil.

In a further advantageous embodiment of the process, the nucleic acid molecules can be expressed in single-celled plant cells (such as algae), see Falciatore et al., Marine

Biotechnology 1 (3), 239 (1999), and references cited therein, and plant cells from higher plants (for example spermatophytes such as crops). Examples of plant expression vectors encompass those which are described in detail herein or in D. Becker, Plant Mol. Biol. 20,1195 (1992), M.W Bevan, [Nucl. Acids Res. 12, 8711(1984), and in “Vectors for Gene Transfer in Higher Plants” in: Transgenic Plants, Vol. 1, Engineering and Utilization, eds. S. D. Kung and R. Wu, Academic Press, 1993, pp. 15-38]. An overview of binary vectors and their use is also found in R. Hellens, Trends in Plant Science,5 (10), 446 (2000).

Vector DNA can be introduced into prokaryotic or eukaryotic cells via conventional transformation or transfection techniques. The terms “transformation” and “transfection” include conjugation and transduction and, as used in the present context, are intended to encompass a multiplicity of prior-art methods for introducing foreign nucleic acid molecules (for example DNA) into a host cell, including calcium phosphate coprecipitation or calcium chloride coprecipitation, DEAE-dextran-mediated transfection, PEG-mediated transfection, lipofection, natural competence, chemically mediated transfer, electroporation or particle bombardment. Suitable methods for the transformation or transfection of host cells, including plant cells, can be found in Sambrook et al. (Molecular Cloning: A Laboratory Manual., 2^{nd} Ed., Cold Spring Harbor Laboratory, Cold Spring Harbor Laboratory Press, Cold Spring Harbor, N.Y., 1989) and in other laboratory handbooks such as Methods in Molecular Biology, 1995, Vol. 44, Agrobacterium protocols, eds. Gartland and Davey, Humana Press, Totowa, N.J.

The above-described methods for the transformation and, if desired, regeneration of plants from plant cells, plant tissues or parts of a plant are exploited for transient or stable transformation of plants. Suitable methods are the transformation of protoplasts by polyethylene-glycol-induced DNA uptake, the biolistic method with the gene gun—known as the particle bombardment method—, electroporation, the incubation of dry embryos in DNA-containing solution, microinjection and the Agrobacterium-mediated gene transfer. The above-mentioned methods are described for example in B. Jenes, Techniques for Gene Transfer, in: Transgenic Plants, Vol. 1, Engineering and Utilization, edited by Kung S.D. and Wu R., Academic Press, pp. 128-143 (1993) and in Potrykus, Annu. Rev. Plant Physiol. Plant Molec. Biol. 42, 205 (1991). The construct to be expressed is preferably cloned into a vector, which is suitable for transforming Agrobacterium tumefaciens, for example pBin19 (Bevan, Nucl. Acids Res. 12, 8711 (1984)). Agrobacteria transformed with such a vector can then be used in the known manner for the transformation of plants, in particular crop plants, such as, for example, tobacco plants, for example by bathing scarified leaves or leaf segments in an agrobacterial solution and subsequently culturing them in suitable media. The transformation of plants with Agrobacterium tumefaciens is described for example by Hofgen and Willmitzer in Nucl. Acid Res. 16, 9877 (1988) or known from, inter alia, White F.F., “Vectors for Gene Transfer in Higher Plants” in Transgenic Plants, Vol. 1, Engineering and Utilization, edited by Kung S. D. and Wu R., Academic Press, 1993, pp. 15-38. Alternatively the construct to be expressed can be cloned into vectors suitable for plastid transformation, as for example described in WO 2004/029256, WO 20040/04445 or Dufourmantel et al., Plant Mol. Biol. 55, 479 (2004).

To select for the successful transfer of the nucleic acid molecule, vector or nucleic acid construct of the invention according to the invention into a host organism, it is advantageous to use marker genes as have already been described above in detail. It is known of the stable or transient integration of nucleic acids into plant cells that only a minority of the cells takes up the foreign DNA and, if desired, integrates it into its genome, depending on the expression vector used and the transfection technique used. To identify and select these integrants, a gene encoding for a selectable marker (as described above, for example resistance to antibiotics) is usually introduced into the host cells together with the gene of interest. Preferred selectable markers in plants comprise those, which confer resistance to an herbicide such as glyphosate or gluphosinate. Other suitable markers are, for example, markers, which encode genes involved in biosynthetic pathways of, for example, sugars or amino acids, such as R-galactosidase, ura3 or ilv2. Markers, which encode genes such as luciferase, gfp or other fluorescence genes, are likewise suitable. These markers and the aforementioned markers can be used in mutants in which these genes are not functional since, for example, they have been deleted by conventional methods. Furthermore, nucleic acid molecules, which encode a selectable marker, can be introduced into a host cell on the same vector as those, which encode the polypeptides of the invention or used in the process or else in a separate vector. Cells which have been transfected stably with the nucleic acid introduced can be identified for example by selection (for example, cells which have integrated the selectable marker survive whereas the other cells die).

Since the marker genes, as a rule specifically the gene for resistance to antibiotics and herbicides, are no longer required or are undesired in the transgenic host cell once the nucleic acids have been introduced successfully, the process according to the invention for introducing the nucleic acids advantageously employs techniques which enable the removal, or excision, of these marker genes. One such a method is what is known as cotransformation. The cotransformation method employs two vectors simultaneously for the transformation, one vector bearing the nucleic acid according to the invention and a second bearing the marker gene(s). A large proportion of transformants receives or, in the case of plants, comprises (up to 40% of the transformants and above), both vectors. In case of transformation with Agrobacteria, the transformants usually receive only a part of the vector, the sequence flanked by the T-DNA, which usually represents the expression cassette. The marker genes can subsequently be removed from the transformed plant by performing crosses. In another method, marker genes integrated into a transposon are used for the transformation together with desired nucleic acid (known as the Ac/Ds technology). The transformants can be crossed with a transposase resource or the transformants are transformed with a nucleic acid construct conferring expression of a transposase, transiently or stable. In some cases (approx. 10%), the transposon jumps out of the genome of the host cell once transformation has taken place successfully and is lost. In a further number of cases, the transposon jumps to a different location. In these cases, the marker gene must be eliminated by performing crosses. In microbiology, techniques were developed which make possible, or facilitate, the detection of such events. A further advantageous method relies on what are known as recombination systems, whose advantage is that elimination by crossing can be dispensed with. The best-known system of this type is what is known as the Cre/lox system. Crel is a recombinase, which removes the sequences located between the loxP sequences. If the marker gene is integrated between the loxP sequences, it is removed, once transformation has taken place successfully, by expression of the recombinase. Further recombination systems are the HI N/H IX, FLP/FRT and REP/STB system (Tribble et al., J. Biol. Chem., 275, 22255 (2000); Velmurugan et al., J. Cell Biol., 149, 553 (2000)). A site-specific integration into the plant genome of the nucleic acid sequences according to the invention is possible. Naturally, these methods can also be applied to microorganisms such as yeast, fungi or bacteria. Also methods for the production of marker-free plastid transformants using a transiently cointegrated selection gene have been described for example by Koop et al., Nature Biotechology, 22 (2), 225 (2004).

Agrobacteria transformed with an expression vector according to the invention may also be used in the manner known per se for the transformation of plants such as experimental plants like Arabidopsis or crop plants, such as, for example, cereals, maize, oats, rye, barley, wheat, soya, rice, cotton, sugarbeet, canola, sunflower, flax, hemp, potato, tobacco, tomato, carrot, bell peppers, oilseed rape, tapioca, cassava, arrow root, tagetes, alfalfa, lettuce and the various tree, nut, and grapevine species, in particular oil-containing crop plants such as soya, peanut, castor-oil plant, sunflower, maize, cotton, flax, oilseed rape, coconut, oil palm, safflower (Carthamus tinctorius) or cocoa beans, or in particular crop plants, like cereals, maize, oats, rye, barley, wheat, soya, rice, cotton, sugarbeet, canola, sunflower, potato or oilseed rape, for example by bathing scarified leaves or leaf segments in an agrobacterial solution and subsequently growing them in suitable media.

In addition to the transformation of somatic cells, which then have to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic (Feldman K. A. and Marks M. D., Mol. Gen. Genet. 208, 274 (1987), Feldmann K . in: Koncz C., Chua N.-H. and Shell J., eds., “Methods in Arabidopsis Research”, Word Scientific, Singapore, (1992) pp. 274-289). Alternative methods are based on the repeated removal of the influorescences and incubation of the excision site in the center of the rosette with transformed agrobacteria, whereby transformed seeds can likewise be obtained at a later point in time (Chang, Plant J., 5, 551 (1994), Katavic, Mol. Gen. Genet. 245, 363 (1994)). However, an especially effective method is the vacuum infiltration method with its modifications such as the “floral dip” method. In the case of vacuum infiltration of Arabidopsis, intact plants under reduced pressure are treated with an agrobacterial suspension (Bechthold N., C. R. Acad. Sci. Paris Life Sci, 316, 1194 (1993)), while in the case of the” floral dip” method the developing floral tissue is incubated briefly with a surfactanttreated agrobacterial suspension (Clough S. J., and Bent A. F., Plant J. 16, 735 (1998)). A certain proportion of transgenic seeds are harvested in both cases, and these seeds can be distinguished from non-transgenic seeds by growing under the above-described selective conditions. In addition the stable transformation of plastids is of advantages because plastids are inherited maternally in most crops reducing or eliminating the risk of transgene flow through pollen. The transformation of the chloroplast genome is generally achieved by a process, which has been schematically displayed in Klaus et al., (Nature Biotechnology 22 (2), 225 (2004)). Briefly the sequences to be transformed are cloned together with a selectable marker gene between flanking sequences homologous to the chloroplast genome. These homologous flanking sequences direct site specific integration into the plastome. Plastidal transformation has been described for many different plant species and an overview can be taken from Bock R., J. Mol. Biol. 312 (3), 425 (2001) or Maliga P., Trends Biotechnol. 21, 20 (2003). Further biotechnological progress has recently been reported in form of marker free plastid transformants, which can be produced by a transient cointegrated maker gene (Klaus et al., Nature Biotechnology 22 (2), 225 (2004)).

The genetically modified plant cells can be regenerated via all methods with which the skilled worker is familiar. Suitable methods can be found in the above-mentioned publications by Kung S. D. and Wu R., Potrykus or Hofgen and Willmitzer.

Accordingly, the present invention thus also relates to a plant cell comprising the nucleic acid construct according to the invention, the expression cassette according to the invention, the nucleic acid molecule according to the invention or the vector according to the invention, as well as a process to generate such a plant cell.

Accordingly the present invention relates to any cell transgenic for any nucleic acid characterized as part of the invention, e.g. conferring the increase of the respective fine chemical alpha-tocopherol, or gamma-tocopherol in a cell or a non-human organism or a part thereof, e.g. the nucleic acid molecule of the invention, the nucleic acid construct of the invention, the vector of the invention, the expression cassette according to the invention, or a nucleic acid molecule encoding the polypeptide of the invention, e.g. encoding a polypeptide having an activity as the protein as shown in the respective line in Table II, application no. 7, column 3. Due to the above-mentioned activity the respective fine chemical alpha-tocopherol, or gamma-tocopherol content in a cell or a non-human organism is increased. For example, due to modulation or manipulation, the cellular activity is increased, in a preferred embodiment in organelles such as plastids or mitochondria, e.g. due to an increased expression or specific activity or specific targeting of the subject matters of the invention in a cell or a non-human organism or a part thereof especially in organelles such as plastids or mitochondria, or in another embodiment in the cytosol. Transgenic for a polypeptide having a protein or a protein activity means herein that due to modulation or manipulation of the genome, the activity of protein as shown in the respective line in Table II, application no. 7, column 3 or a protein as shown in the respective line in Table II, application no. 7, column 3-like activity is increased in the cell or non-human organism or part thereof, especially in organelles such as plastids or mitochondria, or especially in the cytosol. Examples are described above in context with the process of the invention.

“Transgenic”, for example regarding a nucleic acid molecule, a nucleic acid construct, an expression cassette or a vector comprising said nucleic acid molecule or a non-human organism transformed with said nucleic acid molecule, nucleic acid construct, expression cassette or vector, refers to all those subjects originating by recombinant methods in which either
a) the nucleic acid sequence, or
b) a genetic control sequence linked operably to the nucleic acid sequence, for example a promoter, or
c) (a) and (b);

are not located in their natural genetic environment or have been modified by recombinant methods, an example of a modification being a substitution, addition, deletion, inversion or insertion of one or more nucleotide residues. Natural genetic environment refers to the natural chromosomal locus in the organism of origin, or to the presence in a genomic library. In the case of a genomic library, the natural genetic environment of the nucleic acid sequence is preferably retained, at least in part. The environment flanks the nucleic acid sequence at least at one side and has a sequence of at least 50 bp, preferably at least 500 bp, especially preferably at least 1000 bp, very especially preferably at least 5000 bp, in length.

A naturally occurring expression cassette—for example the naturally occurring combination of the promoter of the gene encoding a protein as shown in the respective line in Table II, application no. 7, column 5 or 8, preferably the coding region thereof, or fragments or homologs thereof with the corresponding encoding gene—becomes a transgenic expression cassette when it is modified by non-natural, synthetic “artificial” methods such as, for example, mutagenesis. Such methods have been described (U.S. Pat. No. 5,565,350; WO 00/15815; also see above).

Further, the plant cell, plant or a part thereof can also be transformed such that further enzymes and proteins are (over)expressed which expression supports an increase of the fine chemical.

However, transgenic also means that the nucleic acids according to the invention are located at their natural position in the genome of a non-human organism, but that the sequence has been modified in comparison with the natural sequence and/or that the regulatory sequences of the natural sequences have been modified. Preferably, transgenic/recombinant is to be understood as meaning the transcription of the nucleic acids used in the process according to the invention occurs at a non-natural position in the genome, that is to say the expression of the nucleic acids is homologous or, preferably, heterologous. This expression can be performed with transiently transformed organism or with stably transformed organism.

Transgenic plants comprising the respective fine chemical(s) synthesized in the process according to the invention can be marketed directly without isolation of the fine chemical synthesized. In the process according to the invention, plants are understood as meaning all plant parts, plant organs such as leaf, stalk, root, tubers or seeds or propagation material or harvested material or the intact plant. In this context, the seed encompasses all parts of the seed such as the seed coats, epidermal cells, seed cells, endosperm or embryonic tissue. The fine chemical produced in the process according to the invention may, however, also be isolated from the plant. In case of alpha-tocopherol, or gamma-tocopherol this can be in free form or bound to proteins. Fine chemical(s) produced by this process can be harvested by harvesting the non-human organisms either from the culture in which they grow or from the field. For example, this can be done via squeezing, grinding and/or extraction, salt precipitation and/or ion-exchange chromatography of the plant parts, preferably the plant seeds, plant fruits, plant tubers and the like.

In a further embodiment, the present invention relates to a process for the generation of a microorganism, comprising the introduction, into the microorganism or parts thereof, of the nucleic acid construct of the invention, or the expression cassette of the invention, or the vector of the invention or the nucleic acid molecule of the invention.

In another embodiment, the present invention relates also to a transgenic microorganism comprising the nucleic acid molecule of the invention, the nucleic acid construct of the invention, or the expression cassette of the invention, or the vector of the invention. Appropriate microorganisms have been described herein before under source organism, preferred are in particular aforementioned strains suitable for the production of fine chemicals.

In principle all microorganisms can be used as host organism especially the ones mentioned under source organism above. It is advantageous to use in the process of the invention transgenic microorganisms such as algae selected from the group of the families Bacillariophyceae, Charophyceae, Chlorophyceae, Chrysophyceae, Craspedophyceae, Euglenophyceae, Prymnesiophyceae, Phaeophyceae, Dinophyceae, Rhodophyceae, Xanthophyceae, Prasinophyceae and its described species and strains. Examples for such algae are the following species Isochrysis galbana, Chaetoceros gracilis, Chaetoceros calcitrans, Tetraselmis suecica, Thalassiosira pseudonana, Pavlova lutheri, Isochrysis sp., Skeletonema costatum, Chroomonas salina, Dunaliella tertiolecta, Chaetoceros simplex, Chaetoceros muelleri, Nannochloropsis sp., Cyclotella sp., Phaeodactylum tricornutum, Tetraselmis chui, Pavlova salina, Dicruteria sp., Tetraselmis levis, Dunaliella perva, Thalassiosira weissfloggii, Chlamydomonas sp., Chlorella vulgaris, Neochloris oleoabundans or Chlorella sp, which are only small overview.

The process of the invention is, when the host organisms are microorganisms, advantageously carried out at a temperature between 0° C. and 95° C., preferably between 10° C. and 85° C., particularly preferably between 15° C. and 75° C., very particularly preferably between 15° C. and 45° C. The pH is advantageously kept at between pH 4 and 12, preferably between pH 6 and 9, particularly preferably between pH 7 and 8, during this. The process of the invention can be operated batch wise, semi batch wise or continuously. A summary of known cultivation methods is to be found in the textbook by Chmiel (Bioprozeβtechnik 1. Einführung in die Bioverfahrenstechnik (Gustav Fischer Verlag, Stuttgart, 1991)) or in the textbook by Storhas (Bioreaktoren and periphere Einrichtungen (Vieweg Verlag, Braunschweig/Wiesbaden, 1994)). The culture medium to be used must meet the requirements of the respective strains in a suitable manner. Descriptions of culture media for various microorganisms are present in the handbook “Manual of Methods for General Bacteriology” of the American Society for Bacteriology (Washington D.C., USA, 1981) and for algae in McLellan et al. (“Maintenance of algae and protozoa”, in “Maintenance of Microorganisms”, eds. Doyle A. and Kirsop B., London pp. 183-208 (1991)), Provasoli et al. (“Artificial media for freshwater algae: problems and suggestions”, in “The Ecology of Algae”, eds. Hartman R. T., Pymatunig Laboratory of Field Biology Special publication 2, University of Pittsburgh, pp. 84-96 (1960)) or Starr R. C. (“Algal cultures-sources and methods of cultivation”, in “Photosynthesis Part A, Methods in Enzymology 23”, eds. San Pietro A., N.Y., pp 29-53 (1971)). These media, which can be employed according to the invention include, as described above, usually one or more carbon sources, nitrogen sources, inorganic salts, vitamins and/or trace elements. Preferred carbon sources are sugars such as mono-, di- or polysaccharides. Examples of very good carbon sources are glucose, fructose, mannose, galactose, ribose, sorbose, ribulose, lactose, maltose, sucrose, raffinose, starch or cellulose. Sugars can also be added to the media via complex compounds such as molasses, or other byproducts of sugar refining. It may also be advantageous to add mixtures of various carbon sources. Other possible carbon sources are oils and fats such as, for example, soybean oil, sunflower oil, peanut oil and/or coconut fat, fatty acids such as, for example, palmitic acid, stearic acid and/or linoleic acid, alcohols and/or polyalcohols such as, for example, glycerol, methanol and/or ethanol and/or organic acids such as, for example, acetic acid and/or lactic acid. Nitrogen sources are usually organic or inorganic nitrogen compounds or materials, which contain these compounds. Examples of nitrogen sources include ammonia in liquid or gaseous form or ammonium salts such as ammonium sulfate, ammonium chloride, ammonium phosphate, ammonium carbonate or ammonium nitrate, nitrates, urea, amino acids or complex nitrogen sources such as corn steep liquor, soybean meal, soybean protein, yeast extract, meat extract and others. The nitrogen sources may be used singly or as a mixture. Inorganic salt compounds, which may be present in the media include the chloride, phosphorus or sulfate salts of calcium, magnesium, sodium, cobalt, molybdenum, potassium, manganese, zinc, copper and iron. For the cultivation of algae the so called soilwater media are preferred. Such media are composed of soil extract, trace element solutions, filtered seawater, a nitrogen source and a buffer substance. Such culture media are well known by the skilled person and are available for example from culture collections such as the culture collection of algae (SAG) at the University of Gottingen, the Culture collection of algae in Coimbra, Portugal (ACOI) or the culture collection of algae (UTEX) in Tex., USA.

For preparing sulfur-containing amino acids, in particular methionine or cystein, it is possible to use as sulfur source inorganic sulfur-containing compounds such as, for example, sulfates, sulfites, dithionites, tetrathionates, thiosulfates, sulfides or else organic sulfur compounds such as mercaptans and thiols.

It is possible to use as phosphorus source phosphoric acid, potassium dihydrogenphosphate or dipotassium hydrogenphosphate or the corresponding sodium-containing salts. Chelating agents can be added to the medium in order to keep the metal ions in solution. Particularly suitable chelating agents include dihydroxyphenols such as catechol or protocatechuate, or organic acids such as citric acid. The fermentation media employed according to the invention for cultivating microorganisms normally also contain other growth factors such as vitamins or growth promoters, which include, for example, biotin, riboflavin, thiamine, folic acid, nicotinic acid, pantothenate and pyridoxine. All media components are sterilized either by heat (1.5 bar and 121° C. for 20 min) or by sterilizing filtration. The components can be sterilized either together or, if necessary, separately. All media components can be present at the start of the cultivation or optionally be added continuously or batch wise. The temperature of the culture is normally between 0° C. and 55° C., preferably at 10° C. to 30° C., and can be kept constant or changed during the experiment. The pH of the medium should be in the range from 3.5 to 8.5, preferably in the range between 5 to 7. The pH for the cultivation can be controlled during the cultivation by adding basic compounds such as sodium hydroxide, potassium hydroxide, ammonia or aqueous ammonia or acidic compounds such as phosphoric acid or sulfuric acid. Foaming can be controlled by employing antifoams such as, for example, fatty acid polyglycol esters. In addition the stability of plasmids can be maintained by adding to the medium suitable substances having a selective effect, for example antibiotics, if the plasmids carry the genetic information to overcome the selective effect. Aerobic conditions are maintained by introducing oxygen or oxygen-containing gas mixtures such as, for example, ambient air into the culture. The temperature of the culture is normally from 20° C. to 45° C. and preferably from 25° C. to 40° C. The culture is continued until formation of the desired product is at a maximum. This aim is normally achieved within 10 hours to 160 hours.

The fermentation broths obtained in this way, normally have a dry matter content of 7.5 to 25% by weight. The fermentation broth can be processed further. Depending on requirements, the biomass can be removed entirely or partly by separation methods, such as, for example, centrifugation, filtration, decantation or a combination of these methods, from the fermentation broth or left completely in it. The fermentation broth can then be thickened or concentrated by known methods, such as, for example, with the aid of a rotary evaporator, thin-film evaporator, falling film evaporator, by reverse osmosis or by nanofiltration. This concentrated fermentation broth can then be worked up by freeze-drying, spray drying, spray granulation or by other processes.

In another embodiment, the present invention relates to a method of producing a transgenic non-human organism, especially a microorganism or a plant, with a FCRP coding nucleic acid, wherein expression of the nucleic acid(s) in the microorganism or a plant confers the production or the increased production of the fine chemical as compared to a wild type non-human organism comprising: (a) transforming a non-human cell with a vector comprising a FCRP encoding nucleic acid, and (b) generating from the non-human cell a transgenic non-human organism with production or enhanced production of the fine chemical.

The present invention relates also to a process according to the present invention whereby the produced fine chemical composition or the produced the fine chemical is isolated.

In this manner, more than 50% by weight, advantageously more than 60% by weight, preferably more than 70% by weight, especially preferably more than 80% by weight, very especially preferably more than 90% by weight, of the fine chemical produced in the process can be isolated. The resulting fine chemical can, if appropriate, subsequently be further purified, if desired mixed with other active ingredients such as vitamins, amino acids, carbohydrates, antibiotics and the like, and, if appropriate, formulated.

However, it is also possible to purify the fine chemical produced further with methods known by a person skilled in the art, like extraction, precipitation, crystallization etc. For this purpose, the product-containing composition is for example subjected to a chromatography on a suitable resin, in which case the desired product or the impurities are retained wholly or partly on the chromatography resin. These chromatography steps can be repeated if necessary, using the same or different chromatography resins. The skilled worker is familiar with the choice of suitable chromatography resins and their most effective use. The purified product can be concentrated by filtration or ultrafiltration and stored at a temperature at which the stability of the product is a maximum.

The identity and purity of the isolated compound(s) can be determined by prior art techniques. These include high performance liquid chromatography (HPLC), spectroscopic methods, mass spectrometry (MS), staining methods, thin-layer chromatography, NIRS, enzyme assay or microbiological assays. These analytical methods are summarized for example in: Patek et al., Appl. Environ. Microbiol. 60, 133 (1994), Malakhova et al., Biotekhnologiya 11, 27 (1996); Schmidt et al., Bioprocess Engineer. 19, 67 (1998), Ullmann's Encyclopedia of Industrial Chemistry (1996) Vol. A27, VCH, Weinheim, pp. 89-90, pp. 521-540, pp. 540-547, pp. 559 566, 575-581 and pp. 581-587, Michal G., “Biochemical Pathways: An Atlas of Biochemistry and Molecular Biology”, John Wiley and Sons (1999); Fallon A. et al. “Applications of HPLC in Biochemistry” in “Laboratory Techniques in Biochemistry and Molecular Biology”, Vol. 17 (1987).

The fine chemical of the invention can for example be analyzed advantageously via HPLC or GC separation methods and detected by MS or MSMS methods. The unambiguous detection for the presence of the fine chemical containing products can be obtained by analyzing recombinant organisms using analytical standard methods: GC, GC-MS or TLC, as described on several occasions by Christie and the references therein (1997, in: Advances on Lipid Methodology, Fourth Edition: Christie, Oily Press, Dundee, 119-169; 1998, Gaschromatographie-Massenspektrometrie-Verfahren [Gas chromatography/mass spectrometric methods], Lipide 33:343-353). The material to be analyzed can be disrupted by sonication, grinding in a glass mill, liquid nitrogen and grinding, cooking, or via other applicable methods; see also Biotechnology of Vitamins, Pigments and Growth Factors, Edited by Erik J. Vandamme, London, 1989, p.96 to 103.

The fine chemical obtained in the process are suitable as starting material for the synthesis of further products of value.

For example, in case of vitamins they can be used in combination with each other or alone for the production of pharmaceuticals, foodstuffs, animal feeds or cosmetics. The respective fine chemical can then be processed further directly into foodstuffs or animal feeds or for other applications, for example according to the disclosures made in U.S. Pat. No. 6,399,059: Thermally stable enzyme composition and method of preparing the same, U.S. Pat. No. 6,361,800: Multivitamin and mineral supplement , U.S. Pat. No. 6,348,200: Cosmetic composition, U.S. Pat. No. 6,338,854: Photoaging skin-care preparation and method of treating wrinkled skin, U.S. Pat. No. 6,323,188: Treatment and prevention of cardiovascular diseases, heart attack, and stroke, primary and subsequent, with help of aspirin and certain vitamins, U.S. Pat. No. 6,299,896: Multi-vitamin and mineral supplement, U.S. Pat. No. 6,262,279: Preparation of tocopherols, U.S. Pat. No. 6,362,221: Compositions containing natural lycopene and natural tocopherol, US 6,358,997: Tocopherol and tocotrienol compositions, U.S. Pat. No. 6,344,573: Process for extraction and concentration of liposoluble vitamins and provitamins, growth factors and animal and vegetable hormones from residues and by-products of industrialized animal and vegetable products, U.S. Pat. No. 6,242,227: Method of vitamin production, U.S. Pat. No. 6,207,187: Compositions based on tocopherols, U.S. Pat. No. 6,177,114: Refining of edible oil rich in natural carotenes and Vitamin E which are expressly incorporated herein by reference. The fermentation broth, fermentation products, plants or plant products can be purified as described in above mentioned applications or by other methods known to the person skilled in the art and described herein below.

Accordingly, the present invention relates a method for the production of a pharmaceuticals, food stuff, animal feeds, nutrients or cosmetics comprising the steps of the process according to the invention, including the isolation of the fine chemical produced or a composition produced comprising the fine chemical, and, if desired, formulating the product with a pharmaceutical acceptable carrier or formulating the product in a form acceptable for an application in agriculture or formulating the product in a form acceptable for an application as food stuff, animal feed, nutrient or cosmetic. A further embodiment according to the invention is the use of the fine chemical produced in the process or of the transgenic non-human organisms in animal feeds, foodstuffs, medicines, food supplements, cosmetics or pharmaceuticals.

In yet another aspect, the invention also relates to harvestable parts and to propagation material of the transgenic plants according to the invention which either contain transgenic plant cells expressing a nucleic acid molecule according to the invention or which contains cells which show an increased cellular activity of the polypeptide of the invention, e.g. an increased expression level or higher activity of the described protein.

Harvestable parts can be in principle any useful parts of a plant, for example, flowers, pollen, seedlings, tubers, leaves, stems, fruit, seeds, roots etc. Propagation material includes, for example, seeds, fruits, cuttings, seedlings, tubers, rootstocks etc. Preferred are seeds, fruits, seedlings or tubers as harvestable or propagation material.

The invention furthermore relates to the use of the transgenic non-human organisms according to the invention and of the cells, cell cultures, parts—such as, for example, roots, leaves and the like as mentioned above in the case of transgenic plant organisms—derived from them, and to transgenic propagation material such as seeds or fruits and the like as mentioned above, for the production of foodstuffs or feeding stuffs, pharmaceuticals or fine chemicals.

Further in another embodiment, the present invention relates to the use of the nucleic acid molecule of the invention or used in the method of the invention alone or in combination with other genes of the respective fine chemical synthesis for example of the vitamin E or its precursor 2,3-dimethyl-5-phytylquinol resp., in particular, of alpha- and/or gamma-tocopherol biosynthesis, the polypeptide of the invention or used in the method of the invention, the nucleic acid construct of the invention, the expression cassette of the invention, the vector of the invention, the plant or plant tissue or the host cell of the invention, for the production of plant resistant to a herbicide inhibiting eventually the production of the fine chemical.

Furthermore preferred is a method for the recombinant production of fine chemicals in non-human host organisms, wherein a non-human host organism is transformed with one of the above-described nucleic acid molecules or nucleic acid constructs, expression cassettes or vectors comprising one or more nucleic acid molecules, wherein said nucleic acid molecule participates in the biosynthesis of the desired fine chemical or encodes a polypeptide that catalyze the biosynthesis of the desired fine chemical, the transformed non-human host organism is cultured, and the desired fine chemical is isolated from the non-human organism and/or culture medium. In another embodiment s the additional production of further amino acids, tocopherols and tocotrienols and/or carotenoids or compositions comprising said compounds and/or of pharmaceuticals is preferred. The transformed non-human host organisms are cultured and the products are recovered from the non-human host organisms and/or the culture medium by methods known to the skilled worker or the non-human organism itself serves as food or feed or food or feed supplement. The production of pharmaceuticals such as, for example, antibodies or vaccines, is described by Hood E.E. et al., J, Curr. Opin. Biotechnol. 10 (4), 382 (1999), Ma J.K., Vine N.D., Curr. Top. Microbiol. Immunol. 236, 275 (1999).

In one embodiment, the present invention relates to a method for the identification of a gene product conferring the production of or an increase in the fine chemical production in a non-human cell, comprising the following steps:
(a) contacting, e.g. hybridizing, a, some or all nucleic acid molecules of a sample, e.g. cells, tissues, plants or microorganisms or a nucleic acid library, which can contain a candidate gene encoding a gene product conferring the production of or an increase in the fine chemical after expression, with the nucleic acid molecule of the present invention as shown in the respective line in column 5 or 8 of Table I A or B of application no. 7, preferably the coding region thereof, or a fragment or homolog thereof;
(b) identifying the nucleic acid molecules, which hybridize under relaxed stringent conditions with the nucleic acid molecule of the present invention in particular to the nucleic acid molecule sequence shown in the respective line in Table I, application no. 7, columns 5 or 8, preferably the coding region thereof, of a fragment or a homolog thereof, preferably in Table I B, application no. 7, columns 5 or 8, preferably the coding region thereof, of a fragment or a homolog thereof, and, optionally, isolating the full length cDNA clone or complete genomic clone;
(c) introducing the candidate nucleic acid molecules or a fragment thereof in host cells, preferably in a plant cell or a microorganism, appropriate for producing the fine chemical;
(d) expressing the identified nucleic acid molecules in the host cells for which the production of or the increased production of the fine chemical is desired;
(e) assaying the fine chemical level in the non-human host cells; and
(f) identifying the nucleic acid molecule and/or its gene product which expression confers the production of or an increase in the fine chemical level in the non-human host cell after expression compared to the wild type.

Relaxed hybridization conditions are: After standard hybridization procedures washing steps can be performed at low to medium stringency conditions usually with washing conditions of 40° -55° C. and salt conditions between 2×SSC and 0.2×SSC with 0.1% SDS in comparison to stringent washing conditions as e.g. 60° -68° C. with 0.1% SDS. Further examples can be found in the references listed above for the stringent hybridization conditions. Usually washing steps are repeated with increasing stringency and length until a useful signal to noise ratio is detected and depend on many factors as the target, e.g. its purity, GC-content, size etc, the probe, e.g. its length, is it a RNA or a DNA probe, salt conditions, washing or hybridization temperature, washing or hybridization time etc.

In another embodiment, the present invention relates to a method for the identification of a gene product conferring the production of or an increase in the fine chemical production in a cell, comprising the following steps:
(a) identifying a nucleic acid molecule of a non-human organism, which is at least 20%, 25%, 30%, 35%, 40%, 50%, 60%, 70%, 80%, 90%, 95%, 96%, 97%, 98%, 99%, 99.5% or more homolog to the nucleic acid molecule encoding a polypeptide comprising the polypeptide molecule as shown in the respective line in column 5 or 8 of Table II, application no. 7, or comprising a consensus sequence or a polypeptide motif as shown in the respective line in column 8 of Table IV, application no. 7, or being encoded by a nucleic acid molecule comprising a polynucleotide as shown in the respective line in column 5 or 8 of Table I, application no. 7, preferably the coding region thereof, or a homologue thereof as described herein, for example via homology search in a data bank;
(b) introducing the candidate nucleic acid molecule in non-human host cells, preferably in a plant cell or a microorganism, appropriate for producing the fine chemical;
(c) expressing the identified nucleic acid molecule in the non-human host cells;
(d) assaying the fine chemical level in the non-human host cells; and
(e) identifying the nucleic acid molecule and its gene product which expression confers the production of or an increase in the fine chemical level in the non-human host cell after expression compared to the wild type.

The nucleic acid molecules identified can then be used for the production of the fine chemical in the same way as the nucleic acid molecule of the present invention. Accordingly, in one embodiment, the present invention relates to a process for the production of the fine chemical, comprising (i) identifying a nucleic acid molecule according to aforementioned steps (a) to (f) or (a) to (e) and (ii) recovering the free or bound fine chemical from a non-human organism having an increased cellular activity of a polypeptide encoded by the nucleic acid molecule compared to a wild type and/or the respective culture medium.

Further, the nucleic acid molecules disclosed herein, in particular the nucleic acid molecules shown in the respective line in column 5 or 8 of Table I A or B of application no. 7, may be sufficiently homologous to the sequences of related species such that these nucleic acid molecules may serve as markers for the construction of a genomic map in related organism or for association mapping. Furthermore natural variation in the genomic regions corresponding to nucleic acids disclosed herein, in particular the nucleic acid molecule shown in the respective line in column 5 or 8 of Table I A or B, of application no. 7 or homologous thereof, may lead to variation in the activity of the proteins disclosed herein, in particular the proteins comprising polypeptides as shown in the respective lines in column 5 or 8 of Table II A or B, of application no. 7 or their homologous, or comprising the consensus sequence or the polypeptide motif as shown in the respective line in column 8 of Table IV, and in consequence in a natural variation of an increased fine chemical production.

In consequence natural variation eventually also exists in form of more active allelic variants leading already to a relative increase in the fine chemical. Different variants of the nucleic acid molecules disclosed herein, in particular the nucleic acid molecules comprising the nucleic acid molecules as shown in the respective line in column 5 or 8 of Table I A or B of application no. 7, preferably the coding region thereof, which corresponds to different levels of increase in fine chemical, e.g. different levels of increase in fine chemical, can be indentified and used for marker assisted breeding for an enhanced production of the fine chemical.

Natural variation may also exist in the regulatory regions, e.g. the promoters of the natural genes comprising the nucleic acid molecules as shown in the respective line column 5 or 8 of Table I A or B of application no. 7, preferably the coding region thereof, which can similarly correspond to different levels of increase in the fine chemical, e.g. different levels of increase in the fine chemical can be identified and used for marker assisted breeding for an enhanced production of the fine chemical.

Accordingly, the present invention relates to a method for breeding plants for the production of the fine chemical, comprising
(a) selecting a first plant variety with an increased production of the fine chemical, e.g. based on an increased expression of a nucleic acid molecule of the invention as disclosed herein, in particular of a nucleic acid molecule comprising a nucleic acid molecule as shown in the respective line in column 5 or 8 of Table I A or B of application no. 7, preferably the coding region thereof, or fragments or homologs thereof, or a polypeptide comprising a polypeptide as shown in the respective line in column 5 or 8 of Table II A or B of application no. 7, or fragments or homologs thereof, or comprising a consensus sequence or a polypeptide motif as shown in the respective line in column 8 of Table IV, or a homolog thereof, as described herein;
(b) associating the level of increased production of the fine chemical, with the expression level or the genomic structure of a gene encoding said polypeptide or said nucleic acid molecule;
(c) crossing the first plant variety with a second plant variety, which significantly differs in its level of fine chemical; and
(d) identifying, which of the offspring varieties has got increased production of the fine chemical by the expression level of said polypeptide or nucleic acid molecule or the genomic structure of the gene(s) encoding said polypeptide or nucleic acid molecule of the invention.

In one embodiment, the expression level of the gene according to step (b) is increased.

In another embodiment, the present invention relates to a method for the identification of a compound stimulating production of the fine chemical in a non-human organism, especially a microorganism or a plant, comprising:
(a) contacting the microorganism or plant cells which express the polypeptide of the present invention or its mRNA with a candidate compound under appropriate conditions or in case of plant cells under cell cultivation conditions;
(b) assaying the production of or an increase in expression of said polypeptide or said mRNA;
(c) comparing the expression level to a standard response made in the absence of said candidate compound; whereby, the production of or an increased expression over the standard indicates that the compound is stimulating production of the fine chemical.

Furthermore, in one embodiment, the present invention relates to a process for the identification of a compound conferring the production of or an increase in the fine chemical production in a non-human organism, especially a plant or microorganism, comprising the steps:
(a) culturing a cell or a part of a plant or a microorganism or maintaining a plant expressing the polypeptide according to the invention or a nucleic acid molecule encoding said polypeptide and a readout system capable of interacting with the polypeptide under suitable conditions which permit the interaction of the polypeptide with said readout system in the presence of a compound or a sample comprising a plurality of compounds and capable of providing a detectable signal in response to the binding of a compound to said polypeptide under conditions which permit the expression of said readout system and the polypeptide of the present invention or used in the process of the invention; and
(b) identifying if the compound is an effective agonist by detecting the presence or absence or increase of a signal produced by said readout system.

The screen for a gene product or an agonist conferring the production of or an increase in the fine chemical can be performed by growth of a non-human organism for example a microorganism in the presence of growth reducing amounts of an inhibitor of the synthesis of the fine chemical. Better growth, e.g. higher dividing rate or high dry mass in comparison to the control, e.g. with a gene product or an agonist conferring the production of or an increase in the fine chemical, under such conditions would identify a gene or gene product or an agonist conferring the production of or an increase in fine chemical.

Said compound may be chemically synthesized or microbiologically produced and/or comprised in, for example, samples, e.g., cell extracts from, e.g., plants, non-human animals or microorganisms, e.g. pathogens. Furthermore, said compound(s) may be known in the art but hitherto not known to be capable of suppressing or activating the polypeptide of the present invention. The reaction mixture may be a cell free extract or may comprise a cell or tissue culture. Suitable set ups for the method of the invention are known to the person skilled in the art and are, for example, generally described in Alberts et al., “Molecular Biology of the Cell”, third edition (1994), in particular Chapter 17. The compounds may be, e.g., added to the reaction mixture, culture medium, injected into the cell or sprayed onto the plant.

If a sample containing a compound is identified in the method of the invention, then it is either possible to isolate the compound from the original sample identified as containing the compound capable of activating or conferring the production of or an increase in the fine chemical in a non-human organism or part thereof, or one can further subdivide the original sample, for example, if it consists of a plurality of different compounds, so as to reduce the number of different compounds per sample and repeat the method with the subdivisions of the original sample. Depending on the complexity of the samples, the steps described above can be performed several times, preferably until the sample identified according to the method of the invention only comprises a limited number of or only one compound(s). Preferably said sample comprises compounds of similar chemical and/or physical properties. Preferably, the compound identified according to the above-described method or a respective “active” derivative thereof is further formulated in a form suitable for the application in plant breeding or plant cell and tissue culture.

The compounds which can be tested and identified according to a method of the invention may be expression libraries, e.g., cDNA expression libraries, peptides, proteins, nucleic acids, antibodies, small organic compounds, hormones, peptidomimetics, PNAs or the like (Milner, Nature Medicine 1, 879 (1995); Hupp, Cell 83, 237 (1995); Gibbs, Cell 79, 193 (1994) and references cited supra). Said compounds can also be functional derivatives or analogues of known inhibitors or activators. Methods for the preparation of chemical derivatives and analogues are well known to those skilled in the art and are described in, for example, Beilstein, Handbook of Organic Chemistry, Springer edition New York Inc., 175 Fifth Avenue, New York, N.Y. 10010 U.S.A. and Organic Synthesis, Wiley, New York, USA. Furthermore, said derivatives and analogues can be tested for their effects according to methods known in the art. Furthermore, peptidomimetics and/or computer aided design of appropriate derivatives and analogues can be used, for example, according to the methods described above. The non-human cell or tissue that may be employed in the method of the invention preferably is a non-human host cell, plant cell or plant tissue of the invention described in the embodiments hereinbefore.

Thus, in a further embodiment the invention relates to a compound obtained or identified according to the method for identifying an agonist of the invention said compound being an agonist of the polypeptide of the present invention or used in the process of the present invention.

Accordingly, in one embodiment, the present invention further relates to a compound identified by the method for identifying a compound of the present invention.

Said compound is, for example, a homolog of the polypeptide of the present invention. Homologs of the polypeptide of the present invention can be generated by mutagenesis, e.g., discrete point mutation or truncation of the polypeptide of the present invention. As used herein, the term “homolog” refers to a variant form of the protein, which acts as an agonist of the activity of the polypeptide of the present invention. An agonist of said protein can retain substantially the same, or a subset, of the biological activities of the polypeptide of the present invention. In particular, said agonist confers the increase of the expression level of the polypeptide of the present invention and/or the expression of said agonist in a non-human organisms or part thereof confers the increase of free and/or bound fine chemical in the non-human organism or part thereof.

In another embodiment, the invention relates to an antibody specifically recognizing the compound or agonist of the present invention.

The invention also relates to a diagnostic composition comprising at least one of the aforementioned nucleic acid molecules, expression cassettes, vectors, proteins, antibodies or compounds of the invention and optionally suitable means for detection.

The diagnostic composition of the present invention is suitable for the isolation of mRNA from a non-human cell and contacting the mRNA so obtained with a probe comprising a nucleic acid probe as described above under hybridizing conditions, detecting the presence of mRNA hybridized to the probe, and thereby detecting the expression of the protein in the non-human cell. Further methods of detecting the presence of a protein according to the present invention comprise immunotechniques well known in the art, for example enzyme linked immunoadsorbent assay. Furthermore, it is possible to use the nucleic acid molecules according to the invention as molecular markers or primers in plant breeding. Suitable means for detection are well known to a person skilled in the art, e.g. buffers and solutions for hybridization assays, e.g. the afore-mentioned solutions and buffers, and further means for Southern-, Western-, Northern- etc. -blots, as e.g. described in Sambrook et al. are known. In one embodiment diagnostic compositions contain PCR primers designed to specifically detect the presence or the expression level of the nucleic acid molecule to be expressed or to be expressed on an enhanced level in the process of the invention, e.g. of the nucleic acid molecule of the invention, or to discriminate between different variants or alleles of the nucleic acid molecule of the invention or nucleic acid molecules the activity of which is to be increased in the process of the invention.

In another embodiment, the present invention relates to a kit comprising the nucleic acid molecule, the vector, the host cell, the polypeptide, or the, viral nucleic acid molecule, antibody, plant cell, plant or part thereof, the harvestable part, the propagation material and/or the compound and/or agonist identified according to the method of the invention.

The compounds of the kit of the present invention may be packaged in containers such as vials, optionally with/in buffers and/or solvents. If appropriate, one or more of the components of said kits might be packaged in one and the same container or in different ones. Additionally or alternatively, one or more of said components might be adsorbed to a solid support, like a nitrocellulose filter, a glass plate, a chip, a nylon membrane or to the well of a micro titer plate. The kit can be used for any of the herein described methods and embodiments, e.g. for the production of the host cells, transgenic plants, pharmaceutical compositions, detection of homologous sequences, identification of agonists, as food or feed or as a supplement thereof or as supplement for the treating of plants, etc. Further, the kit can comprise instructions for the use of the kit for any of said embodiments. In one embodiment said kit comprises further a nucleic acid molecule encoding one or more of the aforementioned protein, and/or an antibody, a vector, a host cell, a plant cell, a plant or a part thereof. In another embodiment said kit comprises PCR primers to detect and increase the nucleic acid molecule to be increased in the process of the invention, e.g. of the nucleic acid molecule of the invention.

In a further embodiment, the present invention relates to a method for the production of a agricultural composition providing the nucleic acid molecule, the vector, or antibody of the present invention, the viral nucleic acid of the invention, or the polypeptide of the invention, the compound or agonist or comprising the steps of the method according to the invention for the identification of said compound or agonist; and formulating the nucleic acid molecule, the vector, or antibody of the present invention, the viral nucleic acid of the invention, the polypeptide of the invention or the agonist, or compound identified according to the methods or processes of the present invention or with use of the subject matters of the present invention in a form applicable as plant agricultural composition.

In another embodiment, the present invention relates to a method for the production of “the fine chemical”-production supporting plant culture composition comprising the steps of the method of the present invention; and formulating the compound identified in a form acceptable as agricultural composition.

Under “acceptable as agricultural composition” is understood, that such a composition optionally comprises auxiliaries which are customarily used for formulating crop protection agents.

In a further embodiment the nucleic acid molecule of the invention, the polypeptide of the invention, the nucleic acid construct of the invention, the expression cassette of the invention, the non-human organisms, the non-human host cell, the microorganisms, the plant, plant cell, or the part thereof, of the invention, the vector of the invention, the agonist identified with the method of the invention, the nucleic acid molecule identified with the method of the present invention, can be used for the production of the fine chemical or of the fine chemical and one or more other vitamines.

Accordingly, the nucleic acid molecule of the invention, or the nucleic acid molecule identified with the method of the present invention or the complement sequences thereof, the polypeptide of the invention, the nucleic acid construct of the invention, the expression cassette of the invention, the non-human organism, the non-human host cell, the microorganism, the plant, plant cell, or the part thereof of the invention, the vector of the invention, the agonist identified with the method of the invention, the antibody of the present invention, can be used for the production of or the increase in the fine chemical in a non-human organism or part thereof, e.g. in a cell.

These and other embodiments are disclosed and encompassed by the description and examples of the present invention. Further literature concerning any one of the methods, uses and compounds to be employed in accordance with the present invention may be retrieved from public libraries, using for example electronic devices. For example the public database “Medline” may be utilized which is available on the Internet, for example under http://www.ncbi.nlm.nih.gov/PubMed/medline.html. Further databases and addresses, such as http://www.ncbi.nlm.nih.gov/, http://www.infobiogen.fr/, http://www.fmi.ch/biology/researchtools. html, hftp://www.tigr.org/, are known to the person skilled in the art and can also be obtained using, e.g., http://www.lycos.com.

The present invention is illustrated by the examples, which follow. The present examples illustrate the basic invention without being intended as limiting the subject of the invention. The content of all of the references, patent applications, patents and published patent applications cited in the present patent application is herewith incorporated by reference.

A special embodiment of this invention is related to genes which increase or generate the production of the fine chemical Vitamin E, alpha- or gamma-tocopherol in plant cells, plants or part thereof. Phenotypes thereto are associated with yield of plants (=yield related phenotypes). In accordance with the invention, therefore, the respective genes identified in Table I, wherein in column 7 alpha- or gamma-tocopherol are mentioned, especially the coding region thereof, or homologs or fragments thereof, may be employed to enhance any yield-related phenotype. Increased yield may be determined in field trials of transgenic plants and suitable control plants. Alternatively, a transgene's ability to increase yield may be determined in a model plant. An increased yield phenotype may be determined in the field test or in a model plant by measuring any one or any combination of the following phenotypes, in comparison to a control plant: yield of dry harvestable parts of the plant, yield of dry aerial harvestable parts of the plant, yield of underground dry harvestable parts of the plant, yield of fresh weight harvestable parts of the plant, yield of aerial fresh weight harvestable parts of the plant yield of underground fresh weight harvestable parts of the plant, yield of the plant's fruit (both fresh and dried), grain dry weight, yield of seeds (both fresh and dry), and the like.

The most basic yield-related phenotype is increased yield associated with the presence of the gene or a homolog or a fragment thereof as a transgene in the plant, i.e., the intrinsic yield of the plant. Intrinsic yield capacity of a plant can be, for example, manifested in a field test or in a model system by demonstrating an improvement of seed yield (e.g. in terms of increased seed/grain size, increased ear number, increased seed number per ear, improvement of seed filling, improvement of seed composition, embryo and/or endosperm improvements, and the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under non-stressed conditions), improvement in plant architecture. Increased yield-related phenotypes may also be measured to determine tolerance to abiotic environmental stress. Abiotic stresses include drought, low temperature, salinity, osmotic stress, shade, high plant density, mechanical stresses, and oxidative stress, and yield-related phenotypes are encompassed by tolerance to such abiotic stresses. Additional phenotypes that can be monitored to determine enhanced tolerance to abiotic environmental stress include, without limitation, wilting; leaf browning; loss of turgor, which results in drooping of leaves or needles stems, and flowers; drooping and/or shedding of leaves or needles; the leaves are green but leaf angled slightly toward the ground compared with controls; leaf blades begun to fold (curl) inward; premature senescence of leaves or needles; loss of chlorophyll in leaves or needles and/or yellowing. Any of the yield-related phenotypes described above may be monitored in field tests or in model plants to demonstrate that a transgenic plant has increased tolerance to abiotic environmental stress. In accordance with the invention, the respective genes identified in Table 1, in case in column 7 alpha- or gamma-tocopherol is indicated, especially the coding region thereof, or homologs or fragments thereof, may be employed to enhance tolerance to abiotic environmental stress in a plant means that the plant, when confronted with abiotic environmental stress.

A polypeptide conferring an yield-increasing activity can be encoded by a respective nucleic acid sequence as shown in Table I, column 5 or 8, and/or comprises or consists of a respective polypeptide as depicted in Table II, column 5 and 8, and/or can be amplified with the respective primer set shown in Table III, column 8, in case in column 7 alpha- or gamma-tocopherol is indicated.

“Improved adaptation” to environmental stress like e.g. freezing and/or chilling temperatures refers to an improved plant performance under environmental stress conditions.

A modification, i.e. an increase, can be caused by endogenous or exogenous factors. For example, an increase in activity in an organism or a part thereof can be caused by adding a gene product or a precursor or an activator or an agonist to the media or nutrition or can be caused by introducing said subjects into an organism, transient or stable. Furthermore such an increase can be reached by the introduction of the respective inventive nucleic acid sequence or the encoded protein in the correct cell compartment for example into the nucleus or cytoplasmic respectively or into plastids either by transformation and/or targeting.

The term “yield” as used herein generally refers to a measurable produce from a plant, particularly a crop. Yield and yield increase (in comparison to a non-transformed starting or wild-type plant) can be measured in a number of ways, and it is understood that a skilled person will be able to apply the correct meaning in view of the particular embodiments, the particular crop concerned and the specific purpose or application concerned. The terms “improved yield” or “increased yield” can be used interchangeable.

As used herein, the term “improved yield” or the term “increased yield” means any improvement in the yield of any measured plant product, such as grain, fruit or fiber. In accordance with the invention, changes in different phenotypic traits may improve yield. For example, and without limitation, parameters such as floral organ development, root initiation, root biomass, seed number, seed weight, harvest index, tolerance to abiotic environmental stress, leaf formation, phototropism, apical dominance, and fruit development, are suitable measurements of improved yield. Any increase in yield is an improved yield in accordance with the invention. For example, the improvement in yield can comprise a 0.1%, 0.5%, 1%, 3%, 5%, 10%, 15%, 20%, 30%, 40%, 50%, 60%, 70%, 80%, 90% or greater increase in any measured parameter. For example, an increase in the bushel/acre yield of soybeans or corn derived from a crop comprising plants which are transgenic for the nucleotides and polypeptides of Table I, in case in column 7 alpha- or gamma-tocopherol is mentioned, as compared with the bushel/acre yield from untreated soybeans or corn cultivated under the same conditions, is an improved yield in accordance with the invention. The increased or improved yield can be achieved in the absence or presence of stress conditions.

For example, enhanced or increased “yield” refers to one or more yield parameters selected from the group consisting of biomass yield, dry biomass yield, aerial dry biomass yield, underground dry biomass yield, fresh-weight biomass yield, aerial fresh-weight biomass yield, underground fresh-weight biomass yield; enhanced yield of harvestable parts, either dry or freshweight or both, either aerial or underground or both; enhanced yield of crop fruit, either dry or fresh-weight or both, either aerial or underground or both; and preferably enhanced yield of seeds, either dry or fresh-weight or both, either aerial or underground or both.

Crop yield is defined herein as the number of bushels of relevant agricultural product (such as grain, forage, or seed) harvested per acre. Crop yield is impacted by abiotic stresses, such as drought, heat, salinity, and cold stress, and by the size (biomass) of the plant. Traditional plant breeding strategies are relatively slow and have in general not been successful in conferring increased tolerance to abiotic stresses. Grain yield improvements by conventional breeding have nearly reached a plateau in maize.

Accordingly, the yield of a plant can depend on the specific plant/crop of interest as well as its intended application (such as food production, feed production, processed food production, biofuel, biogas or alcohol production, or the like) of interest in each particular case. Thus, in one embodiment, yield is calculated as harvest index (expressed as a ratio of the weight of the respective harvestable parts divided by the total biomass), harvestable parts weight per area (acre, square meter, or the like); and the like. The harvest index, i.e., the ratio of yield biomass to the total cumulative biomass at harvest, in maize has remained essentially unchanged during selective breeding for grain yield over the last hundred years. Accordingly, recent yield improvements that have occurred in maize are the result of the increased total biomass production per unit land area. This increased total biomass has been achieved by increasing planting density, which has led to adaptive phenotypic alterations, such as a reduction in leaf angle, which may reduce shading of lower leaves, and tassel size, which may increase harvest index. Harvest index is relatively stable under many environmental conditions, and so a robust correlation between plant size and grain yield is possible. Plant size and grain yield are intrinsically linked, because the majority of grain biomass is dependent on current or stored photosynthetic productivity by the leaves and stem of the plant. As with abiotic stress tolerance, measurements of plant size in early development, under standardized conditions in a growth chamber or greenhouse, are standard practices to measure potential yield advantages conferred by the presence of a transgene.

Acccordingly, the yield of a plant can be increased by improving one or more of the yield-related phenotypes.

Such yield-related phenotypes or traits of a plant the improvement of which results in increased yield comprise, without limitation, the increase of the intrinsic yield capacity of a plant, improved nutrient use efficiency, and/or increased stress tolerance.

For example, yield refers to biomass yield, e.g. to dry weight biomass yield and/or fresh-weight biomass yield. Biomass yield refers to the aerial or underground parts of a plant, depending on the specific circumstances (test conditions, specific crop of interest, application of interest, and the like). In one embodiment, biomass yield refers to the aerial and underground parts. Biomass yield may be calculated as fresh-weight, dry weight or a moisture adjusted basis. Biomass yield may be calculated on a per plant basis or in relation to a specific area (e.g. biomass yield per acre/ square meter/ or the like).

In other embodiment, “yield” refers to seed yield which can be measured by one or more of the following parameters: number of seeds or number of filled seeds (per plant or per area (acre/square meter/ or the like)); seed filling rate (ratio between number of filled seeds and total number of seeds); number of flowers per plant; seed biomass or total seeds weight (per plant or per area (acre/square meter/ or the like); thousand kernel weight (TKW; extrapolated from the number of filled seeds counted and their total weight; an increase in TKW may be caused by an increased seed size, an increased seed weight, an increased embryo size, and/or an increased endosperm). Other parameters allowing to measure seed yield are also known in the art. Seed yield may be determined on a dry weight or on a fresh weight basis, or typically on a moisture adjusted basis, e.g. at 15.5-percent moisture.

For example, the term “increased yield” means that the plant, exhibits an increased growth rate, under conditions of abiotic environmental stress, compared to the corresponding wild-type plant.

An increased growth rate may be reflected inter alia by or confers an increased biomass production of the whole plant, or an increased biomass production of the aerial parts of a plant, or by an increased biomass production of the underground parts of a plant, or by an increased biomass production of parts of a plant, like stems, leaves, blossoms, fruits, and/or seeds. Increased yield includes higher fruit yields, higher seed yields, higher fresh matter production, and/or higher dry matter production.

The term “increased yield” means that the plant, exhibits a prolonged growth under conditions of abiotic environmental stress, as compared to the corresponding, e.g. non-transformed, wild type organism. A prolonged growth comprises survival and/or continued growth of the plant, at the moment when the non-transformed wild type organism shows visual symptoms of deficiency and/or death.

When the plant of the invention is a corn plant increased yield for corn plants means, for example, increased seed yield, in particular for corn varieties used for feed or food. Increased seed yield of corn refers to an increased kernel size or weight, an increased kernel per ear, or increased ears per plant. Alternatively or in addition the cob yield may be increased, or the length or size of the cob is increased, or the kernel per cob ratio is improved.

When the plant of the invention is a soy plant, increased yield for soy plants means increased seed yield, in particular for soy varieties used for feed or food. Increased seed yield of soy refers for example to an increased kernel size or weight, an increased kernel per pod, or increased pods per plant.

When the plant of the invention is an oil seed rape (OSR) plant, increased yield for OSR plants means increased seed yield, in particular for OSR varieties used for feed or food. Increased seed yield of OSR refers to an increased seed size or weight, an increased seed number per silique, or increased siliques per plant.

When the plant of the invention is a cotton plant,increased yield for cotton plants means increased lint yield. Increased lint yield of cotton refers in one embodiment to an increased length of lint.

When the plant of the invention is a wheat plant increased yield for wheat plants means, for example, increased seed yield, in particular for wheat varieties used for feed or food. Increased seed yield of wheat refers to an increased kernel size or weight, an increased kernel per ear, or increased ears per plant. Increased seed yield of wheat refers to an increased grain size or weight, an increased number of grains per spike, or an increased number of spikes per plant. Additionally or as an alternative increased seed yield of wheat refers to an increased 1000 grain weight, an increased length of spikes, or an increased number of spikelets per spikes, or an increased number of spikes per square meter, or or an increased flag leaf area (flag leaf is the leaf directly below the spike).

Said increased yield can typically be achieved by enhancing or improving, one or more yieldrelated traits of the plant. Such yield-related traits of a plant comprise, without limitation, the increase of the intrinsic yield capacity of a plant, improved nutrient use efficiency, and/or increased stress tolerance, in particular increased abiotic stress tolerance.

Intrinsic yield capacity of a plant can be, for example, manifested by improving the specific (intrinsic) seed yield (e.g. in terms of increased seed/ grain size, increased ear number, increased seed number per ear, improvement of seed filling, improvement of seed composition, embryo and/or endosperm improvements, or the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under stressed or non-stressed conditions), improvement in plant architecture, cell cycle modifications, photosynthesis modifications, various signaling pathway modifications, modification of transcriptional regulation, modification of translational regulation, modification of enzyme activities, and the like); and/or the like. The improvement or increase of stress tolerance of a plant can for example be manifested by improving or increasing a plant's tolerance against stress, particularly abiotic stress. In the present application, abiotic stress refers generally to abiotic environmental conditions a plant is typically confronted with, including, but not limited to, drought (tolerance to drought may be achieved as a result of improved water use efficiency), heat, low temperatures and cold conditions (such as freezing and chilling conditions), salinity, osmotic stress , shade, high plant density, mechanical stress, oxidative stress, and the like. The increased plant yield can also be mediated by increasing the “nutrient use efficiency of a plant”, e.g. by improving the use efficiency of nutrients including, but not limited to, phosphorus, potassium, and nitrogen. Further, higher yields may be obtained with current or standard levels of nitrogen use.

Generally, the term “increased tolerance to stress” can be defined as survival of plants, and/or higher yield production, under stress conditions as compared to a non-transformed wild type or starting plant: For example, the plant of the invention or produced according to the method of the invention is better adapted to the stress conditions. “Improved adaptation” to environmental stress like e.g. drought, heat, nutrient depletion, freezing and/or chilling temperatures refers herein to an improved plant performance resulting in an increased yield, particularly with regard to one or more of the yield related traits as defined in more detail above.

During its life-cycle, a plant is generally confronted with a diversity of environmental conditions. Any such conditions, which may, under certain circumstances, have an impact on plant yield, are herein referred to as “stress” condition. Environmental stresses may generally be divided into biotic and abiotic (environmental) stresses. Unfavorable nutrient conditions are sometimes also referred to as “environmental stress”. The present invention does also contemplate solutions for this kind of environmental stress, e.g. referring to increased nutrient use efficiency. For the purposes of the description of the present invention, the terms “enhanced tolerance to abiotic stress”, “enhanced resistance to abiotic environmental stress”, “enhanced tolerance to environmental stress”, “improved adaptation to environmental stress” and other variations and expressions similar in its meaning are used interchangeably and refer, without limitation, to an improvement in tolerance to one or more abiotic environmental stress(es) as described herein and as compared to a corresponding origin or wild type plant or a part thereof.

The term abiotic stress tolerance(s) refers for example low temperature tolerance, drought tolerance or improved water use efficiency (WUE), heat tolerance, salt stress tolerance and others. Studies of a plant's response to desiccation, osmotic shock, and temperature extremes are also employed to determine the plant's tolerance or resistance to abiotic stresses. Water use efficiency (WUE) is a parameter often correlated with drought tolerance. In selecting traits for improving crops, a decrease in water use, without a change in growth would have particular merit in an irrigated agricultural system where the water input costs were high. An increase in growth without a corresponding jump in water use would have applicability to all agricultural systems. In many agricultural systems where water supply is not limiting, an increase in growth, even if it came at the expense of an increase in water use also increases yield. Drought stress means any environmental stress which leads to a lack of water in plants or reduction of water supply to plants, including a secondary stress by low temperature and/or salt, and/or a primary stress during drought or heat, e.g. desiccation etc.

Accordingly, this invention provides respective measures and methods to produce plants with increased yield, e.g. genes conferring an increased yield-related trait, for example enhanced tolerance to abiotic environmental stress, for example an increased drought tolerance and/or low temperature tolerance, intrinsic yield and/or another increased yield-related trait, upon expression or over-expression. Accordingly, the present invention provides such genes in case in column 7 of the respective Table or alpha- or gamma-tocopherol is indicated. In particular, such genes are described in column 5 as well as in column 8 of Tables I, especially the coding region thereof, or homologs or fragments thereof, in case alpha- or gamma-tocopherol are indicated in column 7 or the respective polypeptides are described in column 5 as well as in column 8 of Table II, or homologs or fragments thereof, in case alpha- or gamma-tocopherol are indicated in column 7.

Accordingly, the present invention provides respective transgenic plants showing one or more improved yield-related traits as compared to the corresponding origin or the wild type plant and methods for producing such transgenic plants with increased yield in case in column 7 of the respective Table “alpha-tocopherol” or “gamma-tocopherol” is indicated.

In one embodiment, one or more of said yield-increasing activities are increased by increasing the amount and/or the specific activity of one or more proteins listed in Table II, column 5 or 8 in a compartment of a cell indicated in Table II, column 6, in case in column 7 of the respective Table “alpha-tocopherol” or “gamma-tocopherol” is indicated.

Accordingly to present invention, the yield of the plant of the invention is increased by improving one or more of the yield-related traits as defined herein. Said increased yield in accordance with the present invention can typically be achieved by enhancing or improving, in comparison to an origin or wild-type plant, one or more yield-related traits of said plant. Such yield-related traits of a plant the improvement of which results in increased yield comprise, without limitation, the increase of the intrinsic yield capacity of a plant, improved nutrient use efficiency, and/or increased stress tolerance, preferably an increased drought tolerance and/or low temperature tolerance.

In one embodiment, the present invention provides respective transgenic plants showing one or more improved yield-related traits, preferably increased drought tolerance as compared to the corresponding origin or the wild type plant and methods for producing such transgenic plants with increased yield in case in column 7 of the respective Table “alpha-tocopherol” or “gamma-tocopherol” is indicated.

In one embodiment, the present invention provides respective transgenic plants showing one or more improved yield-related traits, preferably increased stress tolerance against low temperatures and cold conditions (such as freezing and chilling conditions) as compared to the corresponding origin or the wild type plant and methods for producing such transgenic plants with increased yield in case in column 7 of the respective Table “alpha-tocopherol” or “gamma-tocopherol” is indicated.

In a further embodiment the present invention is characterized by the features as disclosed in the following items:

Item 1. A process for the production of a fine chemical selected from the group consisting of:
alpha-tocopherol and/or gamma-tocopherol
(a) increasing or generating one or more activities selected from the group consisting of 3-deoxy-7-phosphoheptulonate synthase, 3-deoxy-D-arabinoheptulosonate-7-phosphatesynthase, ABC transporter permease subunit, acetyltransferase, acid shock protein, adenylate kinase, adenylosuccinate lyase, amino-acid acetyltransferase, ankyrin repeat family protein, At1g29350-protein, ATP-binding component of a transport system, b1163-protein, b1219-protein, b1234-protein, b1522-protein, b1921-protein, b2360-protein, b2548-protein, b2613-protein, b2673-protein, b2812-protein, b3890-protein, b3989-protein, b4029-protein, b4050-protein, b4056-protein, calcium-dependent protein kinase, carbohydrate kinase, colanic acid biosynthesis protein, coproporphyrinogen oxidase, cyclin D , DNA polymerase subunit, electron transfer flavoprotein subunit beta, geranylgeranyl pyrophosphate synthase, geranyltranstransferase, gluconate transport system permease 3, glucose dehydrogenase, glucose-6-phosphate 1-dehydrogenase, glutaredoxin, glycerol dehydrogenase, glycerol-3-phosphate dehydrogenase, glycolate utilization protein, harpin-induced family protein, homocitrate synthase, hydrolase, isopropylmalate isomerase large subunit, lipopolysaccharide biosynthesis protein, MADS box protein transcription factor, major facilitator superfamily transporter protein, malate dehydrogenase, monothiol glutaredoxin, monthiol glutaredoxin, multiple antibiotic resistance protein, murein transglycosylase, N-(5′-phosphoribosyl)anthranilate isomerase, NAD-dependent epimerase/dehydratase, NADP-dependent malic enzyme, O-antigen chain length determinant, phosphatidylserine decarboxylase, phospho-2-dehydro-3-deoxyheptonate aldolase, phosphoadenosine phosphosulfate reductase , phosphofructokinase, photosystem II protein, polygalacturonase, polyphosphate kinase, precorrin methylase, protein kinase, protein phosphatase, putative transport system permease protein, pyruvate kinase, RNA-binding protein, Sec-independent protein translocase subunit, serine protease, sll1761-protein, slr1917-protein, small nuclear ribonucleoprotein , ThiF family protein, thioredoxin, thioredoxin family protein, threonine synthase, transcription factor, transcriptional regulator, transport protein, trehalose-phosphatase, TTC1386-protein, ycl049c-protein, ydl235c-protein, ydr183w-protein, yer106w-protein, ylr178c-protein, ypr170c-protein, and zinc finger protein, in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of alpha-tocopherol and/or gamma-tocopherol or a composition comprising alpha-tocopherol and/or gamma-tocopherol in said non-human organism or in the culture medium surrounding said non-human organism.

Item 2. A process for the production of a fine chemical selected from the group consisting of: alpha-tocopherol and/or gamma-tocopherol, which comprises
(A)
   (i) increasing or generating of the expression of; and/or
   (ii) increasing or generating the expression of an expression product of; and/or
   (iii) increasing or generating one or more activities of an expression product encoded by;
   at least one respective nucleic acid molecule comprising a nucleic acid molecule selected from the group consisting of:
   (a) a nucleic acid molecule encoding the polypeptide shown in column 5 or 8 of Table II, preferably Table II B, application no.7, or a homolog or a fragment thereof;
   (b) a nucleic acid molecule shown in column 5 or 8 of Table I, preferably Table I B, application no.7, or a homolog or a fragment thereof (preferably the coding region thereof);
   (c) a nucleic acid molecule, which, as a result of the degeneracy of the genetic code, can be derived from a polypeptide sequence depicted in column 5 or 8 of Table II, preferably Table II B, application no.7;
   (d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 7, or the coding region thereof;
   (e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no.7;
   (i) a nucleic acid molecule encoding a polypeptide having the activity represented by a respective protein as depicted in column 5 of Table II, application no. 7;
   (j) a nucleic acid molecule which comprises a polynucleotide, which is obtained by amplifying a cDNA library or a genomic library using the primers as depicted in column 8 of Table III, application no. 7; and
   (k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
   or a nucleic acid molecule comprising a sequence which is complementary thereto; in a non-human organism or a part thereof, preferably a microorganism, a plant cell, a plant or a part thereof, as compared to a corresponding non-transformed wild type non-human organism or a part thereof; and
(B) growing the non-human organism or a part thereof under conditions which permit the production of alpha-tocopherol and/or gamma-tocopherol or a composition comprising alpha-tocopherol and/or gamma-tocopherol in said non-human organism or in the culture medium surrounding said non-human organism.

Item 3. A process of items 1 or 2, comprising of recovering alpha-tocopherol and/or gamma-tocopherol in its free or bound form.

Item 4. The process of any one of items 1 to 3, comprising the following steps:
(a) selecting a non-human organism or a part thereof expressing a polypeptide encoded by the nucleic acid molecule characterized in item 2;
(b) mutagenizing the selected non-human organism or the part thereof;
(c) comparing the activity or the expression level of said polypeptide in the mutagenized non-human organism or the part thereof with the activity or the expression of said polypeptide of the selected non-human organisms or the part thereof;

(d) selecting the mutated non-human organisms or parts thereof, which comprise an increased activity or expression level of said polypeptide compared to the selected non-human organism or the part thereof;
(e) optionally, growing and cultivating the non-human organisms or the parts thereof; and
(f) recovering, and optionally isolating, the free or bound alpha-tocopherol and/or gamma-tocopherol produced by the selected mutated non-human organisms or parts thereof.

Item 5. The process of any one of items 1 to 4 wherein the activity of said protein or the expression of said nucleic acid molecule is increased or generated transiently or stably.

Item 6. An isolated nucleic acid molecule comprising a nucleic acid molecule selected from the group consisting of:
(a) a nucleic acid molecule encoding a polypeptide shown in Table II, application no. 7, column 5 or 8, preferably shown in Table II B, application no. 7, column 8, or a homolog or a fragment thereof;
(b) a nucleic acid molecule shown in Table I, application no. 7, column 5 or 8, preferably shown in Table I B, application no. 7, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 7, preferably in column 8 of Table II B, application no. application no.;
(d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 3, preferably shown in column 8 of Table I B, application no. 7, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 7;
(i) a nucleic acid molecule encoding a polypeptide having the activity represented by a respective protein as depicted in column 5 of Table II, application no. 7;
(j) a nucleic acid molecule comprising a polynucleotide which is obtained by amplifying a cDNA library or a genomic library using the primers in Table III, application no. 7, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (i), or a nucleic acid molecule comprising a sequence which is complementary thereto.

Item 7. A nucleic acid construct which confers the expression of the nucleic acid molecule as defined in item 6, comprising one or more regulatory elements.

Item 8. An expression cassette comprising
(a) a promoter, preferably selected from the group consisting of Big35S, PCUbi, Super and USP;
(b) a nucleic acid molecule as defined in item 6.

Item 9. A vector comprising the nucleic acid molecule as defined in item 6 or the nucleic acid construct as claimed in item 7 or the expression cassette as claimed in item 8.

Item 10. A host cell or a plant organelle, which has been transformed with the vector as claimed in item 9 or the nucleic acid molecule as defined in item 6 or the nucleic acid construct as claimed in item 7 or the expression cassette as claimed in item 8.

Item 11. The host cell of item 10, which is a plant cell or a microorganism.

Item 12. A process for producing a polypeptide, wherein the polypeptide is expressed in a host cell as claimed in item 10 or 11.

Item 13. A polypeptide produced by the process as claimed in item 12 or encoded by the nucleic acid molecule as defined in item 6.

Item 14. An antibody, which binds specifically to the polypeptide as claimed in item 13.

Item 15. A plant tissue, propagation material, harvested material or a plant comprising the host cell as claimed in item 10 or 11.

Item 16. A process for the identification of a compound conferring an increase in alpha-tocopherol and/or gamma-tocopherol production in a non-human organism, comprising the steps:
(a) culturing a plant cell or tissue or microorganism or maintaining a plant expressing the polypeptide encoded by the nucleic acid molecule as defined in item 6 conferring an increase in the amount of alpha-tocopherol and/or gamma-tocopherol in a non-human organism or a part thereof and a readout system capable of interacting with the polypeptide under suitable conditions which permit the interaction of the polypeptide with said readout system in the presence of a compound or a sample comprising a plurality of compounds and capable of providing a detectable signal in response to the binding of a compound to said polypeptide under conditions which permit the expression of said readout system and of the polypeptide encoded by the nucleic acid molecule of item 6 conferring an increase in the amount of alpha-tocopherol and/or gamma-tocopherolin a non-human organism or a part thereof;
(b) identifying if the compound is an effective agonist by detecting the presence or absence or increase of a signal produced by said readout system.

Item 17. A method for the production of an agricultural composition comprising the steps of the method of item 16 and formulating the compound identified in item 16 in a form acceptable for an application in agriculture.

Item 18. A composition comprising the nucleic acid molecule as defined in item 6, the polypeptide as claimed in item 13, the nucleic acid construct as claimed in item 7, the expression cassette as claimed in item 8, the vector as claimed in item 9, the compound as claimed in item 16, the antibody as claimed in item 14, and optionally an agricultural acceptable carrier.

Item 19. Use of the nucleic acid molecule as defined in item 6 for the identification of a nucleic acid molecule conferring an increase in alpha-tocopherol and/or gamma-tocopherol after expression.

Item 20. Cosmetic, pharmaceutical, food or feed composition comprising the nucleic acid moleculeas defined in item 6, the polypeptide as claimed in item 13, the nucleic acid construct as claimed in item 7, the expression cassette as claimed in item 8, the vector as claimed in item 9, the antibody as claimed in item 14 , the plant or plant tissue as claimed in item 15, the harvested material as claimed in item 15 or the host cell of item as claimed in items 10 or 11.

Item 21. Use of the nuclei acid molecule as defined in item 6, the polypeptide as claimed in item 13, the nucleic acid construct as claimed in item 7, the expression cassette as claimed in item 8, the vector as claimed in item 9, the plant or plant tissue as claimed in item 15, or the host cell of item 10 to 11 for the production of plant resistant to a herbicide inhibiting the production of alpha-tocopherol and/or gamma-tocopherol.

Item 22. A method for producing a plant with increased yield as compared to a corresponding non-transformed wild type plant, whereby the method comprises
(i) increasing or generating of the expression of; and/or
(ii) increasing or generating the expression of an expression product of; and/or
(iii) increasing or generating one or more activities of an expression product encoded by;
at least one respective nucleic acid molecule comprising a nucleic acid molecule selected from the group consisting of:
(a) a nucleic acid molecule encoding the polypeptide shown in column 5 or 8 of Table II, preferably Table II B, application no. 7, or a homolog or a fragment thereof, in case in column 7 alpha-tocopherol and/or gamma-tocopherol is indicated;
(b) a nucleic acid molecule shown in column 5 or 8 of Table I, preferably Table I B, application no. 7, or a homolog or a fragment thereof (preferably the coding region thereof), in case in column 7 alpha-tocopherol and/or gamma-tocopherol is indicated,
(c) a nucleic acid molecule, which, as a result of the degeneracy of the genetic code, can be derived from a polypeptide sequence depicted in column 5 or 8 of Table II, preferably Table II B, application no. 7, in case in column 7 alpha-tocopherol and/or gamma-tocopherolis indicated;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 7, or the coding region thereof, in case in column 7 alpha-tocopherol and/or gamma-tocopherol is indicated;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 7, in case in column 7 alpha-tocopherol and/or gamma-tocopherol is indicated;
(i) a nucleic acid molecule encoding a polypeptide having the activity represented by a respective protein as depicted in column 5 of Table II, application no. 7, in case in column 7 alpha-tocopherol and/or gamma-tocopherol is indicated;
(j) a nucleic acid molecule which comprises a polynucleotide, which is obtained by amplifying a cDNA library or a genomic library using the primers as depicted in column 8 of Table III, application no. 7, in case in column 7 alpha-tocopherol and/or gamma-tocopherol is indicated; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto; in a plant cell, plant or a part thereof.

Item 23. A method for producing a plant with increased yield according to item 22, which comprises in addition the regeneration of a plant from such transformed plant cell, plant cell compartment, plant cell nucleus, plant cell or plant tissue with increased yield.

### EXAMPLES

### Example 1

### Cloning of the Sequences as Shown in Table I, Column 5 or 8 in Escherichia Coli

The inventive sequences as shown in the respective line in Table I, column 5 or 8 were cloned into the plasmids pBR322 (Sutcliffe J. G., Proc. Natl. Acad. Sci. USA, 75, 3737 (1979)), pACYC 177 (Change and Cohen, J. Bacteriol. 134, 1141 (1978)), plasmids of the pBS series (pBSSK+, pBSSK− and others; Stratagene, LaJolla, USA) or cosmids such as SuperCosi (Stratagene, LaJolla, USA) or Lorist6 (Gibson T. J., Rosenthal A. and Waterson R. H., Gene 53, 283 (1987) for expression in E. coli using known, well-established procedures (see, for example, J. Sambrook et al. “Molecular Cloning: A Laboratory Manual”. Cold Spring Harbor Laboratory Press (1989) or F. M. Ausubel et al., “Current Protocols in Molecular Biology”, John Wiley & Sons (1994)).

### Example 2

### DNA Sequencing and Computerized Functional Analysis

The DNA was sequenced by standard procedures, in particular the chain determination method, using AB1377 sequencers (see, for example, Fleischman R. D. et al., Science 269, 496 (1995)).

### Example 3

### In-Vivo and In-Vitro Mutagenesis

An in vivo mutagenesis of organisms such as Saccharomyces, Mortierella, Escherichia and others mentioned above, which are beneficial for the production of a fine chemical can be carried out by passing a plasmid DNA (or another vector DNA) containing the desired nucleic acid sequence or nucleic acid sequences through E. coli and other microorganisms (for example Bacillus spp. or yeasts such as Saccharomyces cerevisiae) which are not capable of maintaining the integrity of its genetic information. Usual mutator strains have mutations in the genes for the DNA repair system (for example mutHLS, mutD, mutT and the like; for comparison, see Rupp W. D., “DNA repair mechanisms in Escherichia coli and Salmonella”, pp. 2277-2294, ASM, Washington (1996)]. The skilled worker knows these strains. The use of these strains is illustrated for example in Greener A., and Callahan M., Strategies 7, 32 (1994).

In-vitro mutation methods such as increasing the spontaneous mutation rates by chemical or physical treatment are well known to the skilled person. Mutagens like 5-bromo-uracil, N-methyl-N-nitro-N-nitrosoguanidine (=NTG), ethyl methanesulfonate (=EMS), hydroxylamine and/or nitrous acid are widely used as chemical agents for random in-vitro mutagenesis. The most common physical method for mutagenesis is the treatment with UV irradiation. Another random mutagenesis technique is the error-prone PCR for introducing amino acid changes into proteins. Mutations are deliberately introduced during PCR through the use of error-prone DNA polymerases and special reaction conditions known to a person skilled in the art. For this method randomized DNA sequences are cloned into expression vectors and the resulting mutant libraries screened for altered or improved protein activity as described below.

Site-directed mutagenesis method such as the introduction of desired mutations with an M13 or phagemid vector and short oligonucleotides primers is a well-known approach for site-directed mutagenesis. The clou of this method involves cloning of the nucleic acid sequence of the invention into an M13 or phagemid vector, which permits recovery of single-stranded recombinant nucleic acid sequence. A mutagenic oligonucleotide primer is then designed whose sequence is perfectly complementary to nucleic acid sequence in the region to be mutated, but with a single difference: at the intended mutation site it bears a base that is complementary to the desired mutant nucleotide rather than the original. The mutagenic oligonucleotide is then allowed to prime new DNA synthesis to create a complementary full-length sequence containing the desired mutation. Another site-directed mutagenesis method is the PCR mismatch primer mutagenesis method also known to the skilled person. Dpnl site-directed mutagenesis is a further known method as described for example in the Stratagene Quickchange™ site-directed mutagenesis kit protocol. A huge number of other methods are also known and used in common practice.

Positive mutation events can be selected by screening the organisms for the production of the desired fine chemical.

### Example 4

### DNA transfer between Escherichia Coli, Saccharomyces Cerevisiae and Mortierella Alpina

Shuttle vectors such as pYE22m, pPAC-ResQ, pClasper, pAUR224, pAMH10, pAML10, pAMT10, pAMU10, pGMH10, pGML10, pGMT10, pGMU10, pPGAL1, pPADH1, pTADH1, pTAex3, pNGA142, pHT3101 and derivatives thereof which allow the transfer of nucleic acid sequences between Escherichia coli, Saccharomyces cerevisiae and/or Mortierella alpina are available to the skilled worker. An easy method to isolate such shuttle vectors is disclosed by Soni R. and Murray J. A. H., Nucleic Acid Research, 20 (21), 5852 (1992). If necessary such shuttle vectors can be constructed easily using standard vectors for E. coli (Sambrook J. et al. “Molecular Cloning: A Laboratory Manual”, Cold Spring Harbor Laboratory Press (1989) or Ausubel F. M. et al. “Current Protocols in Molecular Biology”, John Wiley & Sons (1994)) and/or the aforementioned vectors, which have a replication origin for, and suitable marker from, Escherichia coli, Saccharomyces cerevisiae or Mortierella alpina added. Such replication origins are preferably taken from endogenous plasmids, which have been isolated from species used in the inventive process. Genes, which are used in particular as transformation markers for these species are genes for kanamycin resistance (such as those which originate from the Tn5 or Tn 903 transposon) or for chloramphenicol resistance (Winnacker E.L., “From Genes to Clones Introduction to Gene Technology”, VCH, Weinheim (1987)) or for other antibiotic resistance genes such as for G418, gentamycin, neomycin, hygromycin or tetracycline resistance.

Using standard methods, it is possible to clone a gene of interest into one of the abovedescribed shuttle vectors and to introduce such hybrid vectors into the microorganism strains used in the inventive process. The transformation of Saccharomyces can be achieved for example by LiCI or sheroplast transformation (Bishop et al., Mol. Cell. Biol., 6, 3401 (1986), Sherman et al., “Methods in Yeasts in Genetics”, Cold Spring Harbor Lab. Cold Spring Harbor, N.Y. (1982), Agatep et al., Technical Tips Online 1998, 1:51: P01525 or Gietz et al., Methods Mol. Cell. Biol. 5,255 (1995)) or electroporation (Delorme E., Appl. Environ. Microbiol., 55 (9), 2242 (1989)).

If the transformed sequence(s) is/are to be integrated advantageously into the genome of the microorganism used in the inventive process for example into the yeast or fungi genome, standard techniques known to the skilled worker also exist for this purpose. Solinger et al. (Proc. Natl. Acad. Sci. U S A., 15, 8447 (2001)) and Freedman et al. (Genetics, 162, 15 (2002)) teaches a homolog recombination system dependent on rad 50, rad51, rad54 and rad59 in yeasts. Vectors using this system for homologous recombination are vectors derived from the Ylp series. Plasmid vectors derived for example from the 2p-Vector are known by the skilled worker and used for the expression in yeasts. Other preferred vectors are for example pART1, pCHY21 or pEVP11 as they have been described by McLeod et al. (EMBO J., 6, 729 (1987)) and Hoffman et al. (Genes Dev. 5, 561 (1991)) or Russell et al. (J. Biol. Chem. 258, 143 (1983)). Other beneficial yeast vectors are plasmids of the REP, REP-X, pYZ or RIP series.

### Example 5

### Determining the Expression of the Mutant/Transgenic Protein

The observations of the activity of a mutated, or transgenic, protein in a transformed host cell are based on the fact that the protein is expressed in a similar manner and in a similar quantity as the wild-type protein. A suitable method for determining the transcription quantity of the mutant, or transgenic, gene (a sign for the amount of mRNA which is available for the translation of the gene product) is to carry out a Northern blot (see, for example, Ausubel et al., “Current Protocols in Molecular Biology”, Wiley, New York (1988)), where a primer which is designed in such a way that it binds to the gene of interest is provided with a detectable marker (usually a radioactive or chemiluminescent marker) so that, when the total RNA of a culture of the organism is extracted, separated on a gel, applied to a stable matrix and incubated with this probe, the binding and quantity of the binding of the probe indicates the presence and also the amount of mRNA for this gene. Another method is a quantitative PCR. This information detects the extent to which the gene has been transcribed. Total cell RNA can be isolated for example from yeasts or E. coli by a variety of methods, which are known in the art, for example with the Ambion kit according to the instructions of the manufacturer or as described in Edgington et al., Promega Notes Magazine Number 41, 14 (1993).

Standard techniques, such as Western blot, may be employed to determine the presence or relative amount of protein translated from this mRNA (see, for example, Ausubel et al. “Current Protocols in Molecular Biology”, Wiley, New York (1988)). In this method, total cell proteins are extracted, separated by gel electrophoresis, transferred to a matrix such as nitrocellulose and incubated with a probe, such as an antibody, which binds specifically to the desired protein. This probe is usually provided directly or indirectly with a chemiluminescent or colorimetric marker, which can be detected readily. The presence and the observed amount of marker indicate the presence and the amount of the sought mutant protein in the cell. However, other methods are also known.

### Example 6

### Growth of Genetically Modified Organism: Media and Culture Conditions

Genetically modified Yeast, Mortierella or Escherichia coli are grown in synthetic or natural growth media known by the skilled worker. A number of different growth media for Yeast, Mortierella or Escherichia coli are well known and widely available. A method for culturing Mortierella is disclosed by Jang et al. (Bot. Bull. Acad. Sin. 41, 41 (2000)). Mortierella can be grown at 20° C. in a culture medium containing: 10 g/l glucose, 5 g/l yeast extract at pH 6.5. Furthermore Jang et al. teaches a submerged basal medium containing 20 g/l soluble starch, 5 g/l Bacto yeast extract, 10 g/l KNO3, 1 g/l KH2PO4, and 0.5 g/l MgSO₄.7H₂O, pH 6.5.

Said media, which can be used according to the invention usually consist of one or more carbon sources, nitrogen sources, inorganic salts, vitamins and trace elements. Preferred carbon sources are sugars such as mono-, di- or polysaccharides. Examples of very good carbon sources are glucose, fructose, mannose, galactose, ribose, sorbose, ribulose, lactose, maltose, sucrose, raffinose, starch or cellulose. Sugars may also be added to the media via complex compounds such as molasses or other by-products of sugar refining. It may also be advantageous to add mixtures of various carbon sources. Other possible carbon sources are alcohols and/or organic acids such as methanol, ethanol, acetic acid or lactic acid. Nitrogen sources are usually organic or inorganic nitrogen compounds or materials containing said compounds. Examples of nitrogen sources include ammonia gas, aqueous ammonia solutions or ammonium salts such as NH₄Cl, or (NH4)2SO₄, NH₄OH, nitrates, urea, amino acids or complex nitrogen sources such as cornsteep liquor, soybean flour, soybean protein, yeast extract, meat extract and others. Mixtures of the above nitrogen sources may be used advantageously.

Inorganic salt compounds, which may be included in the media comprise the chloride, phosphorus or sulfate salts of calcium, magnesium, sodium, cobalt, molybdenum, potassium, manganese, zinc, copper and iron. Chelating agents may be added to the medium in order to keep the metal ions in solution. Particularly suitable chelating agents include dihydroxyphenols such as catechol or protocatechulate or organic acids such as citric acid. The media usually also contain other growth factors such as vitamins or growth promoters, which include, for example, biotin, riboflavin, thiamine, folic acid, nicotinic acid, panthothenate and pyridoxine. Growth factors and salts are frequently derived from complex media components such as yeast extract, molasses, cornsteep liquor and the like. The exact composition of the compounds used in the media depends heavily on the particular experiment and is decided upon individually for each specific case. Information on the optimization of media can be found in the textbook “Applied Microbiol. Physiology, A Practical Approach” (eds. Rhodes P. M., Stanbury P. F., IRL Press pp. 53-73 (1997)). Growth media can also be obtained from commercial suppliers, for example Standard 1 (Merck) or BHI (Brain heart infusion, DIFCO) and the like.

All media components are sterilized, either by heat (20 min at 1.5 bar and 121° C.) or by filter sterilization. The components may be sterilized either together or, if required, separately. All media components may be present at the start of the cultivation or added continuously or batchwise, as desired.

The culture conditions are defined separately for each experiment. The temperature is normally between 15° C. and 45° C. and may be kept constant or may be altered during the experiment.

The pH of the medium should be in the range from 5 to 8.5, preferably around 7.0, and can be maintained by adding buffers to the media. An example of a buffer for this purpose is a potassium phosphate buffer. Synthetic buffers such as MOPS, HEPES, ACES and the like may be used as an alternative or simultaneously. The culture pH value may also be kept constant during the culture period by addition of, for example, NaOH or NH₄OH. If complex media components such as yeast extract are used, additional buffers are required less since many complex compounds have a high buffer capacity. When using a fermenter for the culture of microorganisms, the pH value can also be regulated using gaseous ammonia.

The incubation period is generally in a range of from several hours to several days. This time period is selected in such a way that the maximum amount of product accumulates in the fermentation broth. The growth experiments, which are disclosed, can be carried out in a multiplicity of containers such as microtiter plates, glass tubes, glass flasks or glass or metal fermenters of various sizes. To screen a large number of clones, the microorganisms should be grown in microtiter plates, glass tubes or shake flasks, either using simple flasks or baffle flasks. 100 ml shake flasks filled with 10% (based on the volume) of the growth medium required are preferably used. The flasks should be shaken on an orbital shaker (amplitude 25 mm) at a rate ranging from 100 to 300 rpm. Evaporation losses can be reduced by maintaining a humid atmosphere; as an alternative, a mathematical correction should be carried out for the evaporation losses.

If genetically modified clones are examined, an unmodified control clone, or a control clone, which contains the basic plasmid without insertion, should also be included in the tests. If a transgenic sequence is expressed, a control clone should advantageously again be included in these tests. The medium is advantageously inoculated to an OD600 of 0.5 to 1.5 using cells which have been grown on agar plates, such as CM plates (10 g/l glucose, 2.5 g/l NaCl, 2 g/I urea, 10 g/l polypeptone, 5 g/l yeast extract, 5 g/l meat extract, 22 g/l agar, pH value 6.8 established with 2M NaOH), which have been incubated at 30° C. The media are inoculated for example by addition of a liquid preculture of seed organism such as E. coli or S. cerevisiae.

### Example 7

### Growth of Genetically Modified Algae: Media and Culture Conditions

### Growing Chlamydomonas:

Chlamydomonas reinhardtii is able to grow under various growth conditions. It is a unicellular alga. The cells of Chlamydomonas reinhardtii can be normally cultured autotrophically in the media mentioned below. Cells of Chlamydomonas reinhardtii can be cultivated at 25° C. under cool-white fluorescence light at 10,000 lux (120 μmol m²s¹ photosynthetically active radiation) as described by Ghirardi et al., Appl. Biochem. Biotechnol. 63, 141 (1997) or Semin et al., Plant. Physiol., 131, 1756 (2003).

### Chlamydomonas Growth Medium:

1 l growth medium is prepared by adding the following volumes of the stock solutions as mentioned below:

1 ml solution A, 5 ml solution B, 1 ml solution C, 1 ml solution D, 3 ml solution E, 3 ml solution F, 1 ml solution G, 1 ml solution H.

| | |
|---|---|
| A) Trace elements solution: | 1 g/l H₃BO₃ |
| | 1 g/l ZnSO₄ × 7 H₂O |
| | 0.3 g/l MnSO₄ × H₂O |
| | 0.2 g/l CoCl₂ × 6 H₂O |
| | 0.2 g/l Na₂MoO₄ × 2 H₂O |
| | 0.04 g/l CuSO₄ |
| B) Na Citrate solution: | 10% w/v Na citrate × 2 H₂O |
| C) Iron solution: | 1% w/v FeCl₃ × 6 H₂O |
| D) Calcium solution: | 5.3% w/v CaCl₂ × H₂O |
| E) Magnesium solution: | 10% w/v MgSO₄ × 7 H₂O |
| F) Ammonium solution: | 10% w/v NH₄NO₃ |
| G) Potassium solution: | 10% w/v KH₂PO₄ |
| H) Dipotassium solution | 10% w/v K₂HPO₄ |

### Bristol's Soil Extract Medium:

Soil extract medium can generally be used for the growth of axenic and xenic algae cultures. The soil extract is prepared by adding a teaspoon of dry garden soil and a pinch of CaCO₃ to 200 ml distilled water and steaming said solution for approximately 2 h on two consecutive days. Afterwards the supernatant is decanted and added to the desired medium.

To 940 ml bristol's solution 40 ml of soil extract medium is added.

### Bristol's Solution:

To 940 ml of distilled water, the following stock solutions are added:

| | | |
|---|---|---|
| 10 ml | NaNO₃ | (25 g/l) |
| 10 ml | CaCl₂ × 2 H₂O | (2.5 g/l) |
| 10 ml | MgSO₄ × 7 H₂O | (7.5 g/l) |
| 10 ml | K₂HPO₄ | (7.5 g/l) |
| 10 ml | KH₂PO₄ | (17.5 g/l) |
| 10 ml | NaCl | (2.5 g/l) |

Amplification and Cloning of DNA from Chlamydomonas Reinhardtii:

The DNA can be amplified by the polymerase chain reaction (PCR) from Chlamydomonas reinhardtii by the method of Howitt Crispin A. (BioTechniques 21, 32 (1996)).

### Fine Chemical Production in Chlamydomonas Reinhardtii:

The fine chemical production can be analysed as mentioned above. The proteins and nucleic acids can be analyzed as mentioned below.

### Example 8

### In-Vitro Analysis of the Function of the Proteins Encoded by the Transformed Sequences

The determination of the activities and kinetic parameters of enzymes is well known in the art. Experiments for determining the activity of a specific modified enzyme must be adapted to the specific activity of the wild-enzyme type, which is well within the capabilities of the skilled worker. Overviews of enzymes in general and specific details regarding the structure, kinetics, principles, methods, applications and examples for the determination of many enzyme activities can be found for example in the following literature: Dixon M. and Webb E. C.: “Enzymes”, Longmans, London (1979); Fersht “Enzyme Structure and Mechanism”, Freeman, New York(1985); Walsh “Enzymatic Reaction Mechanisms” Freeman, San Francisco (1979); Price N. C., Stevens L., “Fundamentals of Enzymology” Oxford Univ. Press, Oxford (1982); Boyer P. D. (ed.), “The Enzymes”, 3rd ed. Academic Press, New York (1983); Bisswanger H., “Enzymkinetik”, 2^{nd} ed. VCH, Weinheim (1994); Bergmeyer H.U., Bergmeyer J., GraRl M., (eds.) “Methods of Enzymatic Analysis”, 3rd rd. Vol. 1-XII, Verlag Chemie: Weinheim (1983-1986); and “Ullmann's Encyclopedia of Industrial Chemistry” Vol. A9, “Enzymes”, VCH, Weinheim, pp. 352-363 (1987).

### Example 9

### Analysis of the Effect of the Nucleic Acid Molecule on the Production of the Fine Chemical

The effect of the genetic modification in plants, fungi, algae, ciliates on the production of a fine chemical can be determined by growing the modified microorganisms for example Chlamydomonas reinhardtii under suitable conditions (such as those described above) and analyzing the medium and/or the cellular components for the increased production of the fine chemical. Such analytical techniques are well known to the skilled worker and encompass spectroscopy, thinlayer chromatography, various types of staining methods, enzymatic and microbiological methods and analytical chromatography such as high-performance liquid chromatography (see, for example, “Ullmann Encyclopedia of Industrial Chemistry”, Vol. A2, pp. 89-90 and pp. 443-613, V C H, Weinheim (1985); Fallon A. et al., “Applications of HPLC in Biochemistry” in “Laboratory Techniques in Biochemistry and Molecular Biology”, Vol. 17 (1987); Rehm et al. “Biotechnology”, Vol. 3, Chapter III: “Product recovery and purification”, pp. 469-714, VCH: Weinheim (1993); Belter P. A. et al. “Bioseparations: downstream processing for Biotechnology”, John Wiley and Sons (1988); Kennedy J. F. and Cabral J. M. S., “Recovery processes for biological Materials”, John Wiley and Sons(1992); Shaeiwitzand J. A., Henry J. D., “Biochemical Separations” in “Ullmann's Encyclopedia of Industrial Chemistry”, Vol. B3; chapter 11, pp. 1-27, VCH, Weinheim (1988); and Dechow, “Separation and purification techniques in biotechnology”, Noyes Publications (1989)).

In addition to the determination of the fermentation end product, other components of the metabolic pathways which are used for the production of the desired compound, such as intermediates and by-products, may also be analyzed in order to determine the total productivity of the organism, the yield and/or production efficiency of the compound. The analytical methods encompass determining the amounts of nutrients in the medium (for example sugars, hydrocarbons, nitrogen sources, phosphate and other ions), determining biomass composition and growth, analyzing the production of ordinary metabolites from biosynthetic pathways and measuring gases generated during the fermentation. Standard methods for these are described in “Applied Microbial Physiology” A Practical Approach, Rhodes P. M. and Stanbury P. F., eds. IRL Press, pp. 103-129; 131-163 and 165-192, and the references cited therein.

### Example 10

### Purification of a Fine Chemical e.g. Vitamin E, like Alpha-, Beta-, or Gamma-Tocopherol or its Precursor 2,3-dimethyl-5-phytylquinol

The unambiguous detection for the presence of vitamin E, like alpha-, beta-, or gamma-tocopherol, or its precursor 2,3-dimethyl-5-phytylquinol can be obtained by analyzing recombinant organisms using analytical standard methods: GC, GC-MS or TLC, as described (1997, in: Advances on Lipid Methodology, Fourth Edition: Christie, Oily Press, Dundee, 119-169; 1998, Gaschromatographie-Massenspektrometrie-Verfahren [Gas chromatography/mass spectrometric methods], Lipide 33:343-353).

The total vitamin E produced in the organism for example in yeasts used in the inventive process can be analysed for example according to the following procedure:

The material such as yeasts, E. coli or plants to be analyzed can be disrupted by sonication, grinding in a glass mill, liquid nitrogen and grinding or via other applicable methods.

Plant material is initially homogenized mechanically by comminuting in a pestle and mortar to make it more amenable to extraction.

A typical sample pretreatment consists of a total lipid extraction using such polar organic solvents as acetone or alcohols as methanol, or ethers, saponification , partition between phases, seperation of non-polar epiphase from more polar hypophasic derivatives and chromatography.

In order to avoid oxidation, the resulting extracts can be analyzed immediate after the extraction with the aid of a Waters Allience 2690 HPLC system. Tocopherols and vitamin E is separated on a reverse phase column (ProntoSil 200-3-C30, Bischoff) with a mobile phase of 100% methanol, and identified by means of a standard (Merck). The fluorescence of the substances (excitation 295 nm, emission 320 nm), which is detected with the aid of a Jasco FP 920 fluorescence detector, can serve as detection system.

If required and desired, further chromatography steps with a suitable resin may follow. Advantageously, the vitamin E, like alpha-, beta-, or gamma-tocopherol, or its precursor 2,3-dimethyl-5phytylquinol, can be further purified with a so-called RTH PLC. As eluent acetonitrile/water or chloroform/acetonitrile mixtures can be used. If necessary, these chromatography steps may be repeated, using identical or other chromatography resins. The skilled worker is familiar with the selection of suitable chromatography resin and the most effective use for a particular molecule to be purified.

### Example 11

### Cloning of the Sequences as Shown in Table I, Column 5 or 8 for the Expression in Plants

### Example 11a

### PCR Amplification of the Sequences

Unless otherwise specified, standard methods as described in Sambrook et al., Molecular Cloning: A laboratory manual, Cold Spring Harbor 1989, Cold Spring Harbor Laboratory Press are used.

The inventive sequences as shown in the respective line in Table I, column 5, preferably the coding region thereof, were amplified by PCR as described in the protocol of the Pfu Ultra, Pfu Turbo or Herculase DNA polymerase (Stratagene). The composition for the protocol of the Pfu Ultra, Pfu Turbo or Herculase DNA polymerase was as follows: 1×PCR buffer (Stratagene), 0.2 mM of each dNTP, 100 ng genomic DNA of Saccharomyces cerevisiae (strain S288C; Research Genetics, Inc., now Invitrogen), Escherichia coli (strain MG1655; E. coli Genetic Stock Center), Synechocystis sp. (strain PCC6803), Azotobacter vinelandii (strain N.R. Smith,16), Thermus thermophilus (HB8) or 50 ng cDNA from various tissues and development stages of Arabidopsis thaliana (ecotype Columbia), Physcomitrella patens, Glycine max (variety Resnick), Brassica napus, Oryza sativa or Zea mays (variety B73, Mo17, A188), 50 pmol forward primer, 50 pmol reverse primer, with or without 1 M Betaine, 2.5 u Pfu Ultra, Pfu Turbo or Herculase DNA polymerase.

The amplification cycles were as follows:

1 cycle of 2-3 minutes at 94-95° C., then 25-36 cycles with 30-60 seconds at 94-95° C., 30-45 seconds at 50-60° C. and 210-480 seconds at 72° C., followed by 1 cycle of 5-10 minutes at 72° C., then 4-16° C.—preferably for Saccharomyces cerevisiae, Escherichia coli, Synechocystis sp., Azotobacter vinelandii, Thermus thermophilus.

In case of Arabidopsis thaliana, Brassica napus, Glycine max, Oryza sativa, Physcomitrella patens, Zea mays the amplification cycles were as follows:

1 cycle with 30 seconds at 94° C., 30 seconds at 61° C., 15 minutes at 72° C., then 2 cycles with 30 seconds at 94° C., 30 seconds at 60° C., 15 minutes at 72° C., then 3 cycles with 30 seconds at 94° C., 30 seconds at 59° C., 15 minutes at 72° C., then 4 cycles with 30 seconds at 94° C., 30 seconds at 58° C., 15 minutes at 72° C., then 25 cycles with 30 seconds at 94° C., 30 seconds at 57° C., 15 minutes at 72° C., then 1 cycle with 10 minutes at 72° C., then finally 4-16° C.

RNAs were generated with the RNeasy Plant Kit according to the standard protocol (Qiagen) and Superscript II Reverse Transkriptase was used to produce double stranded cDNA according to the standard protocol (Invitrogen).

ORF specific primer pairs for the genes to be expressed are shown in the respective line in Table III, column 8. The following adapter sequences were added to Saccharomyces cerevisiae ORF specific primers (see Table III) for cloning purposes:

SEQ ID NO: 20
i) foward primer: 5′-GGAATTCCAGCTGACCACC-3′
SEQ ID NO: 21
ii) reverse primer: 5′-GATCCCCGGGAATTGCCATG-3′

These adaptor sequences allow cloning of the ORF into the various vectors containing the Resgen adaptors, see table column E of Table c.

The following adapter sequences were added to Saccharomyces cerevisiae, Escherichia coli, Synechocystis sp., Azotobacter vinelandii, Thermus thermophilus, Arabidopsis thaliana, Brassica napus, Glycine max, Oryza sativa , Physcomitrella patens, or Zea mays ORF specific primers for cloning purposes:

SEQ ID NO: 22
iii) forward primer: 5′-TTGCTCTTCC-3′
SEQ ID NO: 23
iiii) reverse primer: 5′-TTGCTCTTCG-3′

The adaptor sequences allow cloning of the ORF into the various vectors containing the Colic adaptors, see table column E of Table c.

Therefore for amplification and cloning of Saccharomyces cerevisiae SEQ ID NO: 113598, a primer consisting of the adaptor sequence i) and the ORF specific sequence SEQ ID NO: 113632 and a second primer consisting of the adaptor sequence ii) and the ORF specific sequence SEQ ID NO: 113633 were used.

For amplification and cloning of Escherichia coli SEQ ID NO: 7081, a primer consisting of the adaptor sequence iii) and the ORF specific sequence SEQ ID NO: 7261 and a second primer consisting of the adaptor sequence iiii) and the ORF specific sequence SEQ ID NO: 7262 were used.

For amplification and cloning of Synechocystis sp. SEQ ID NO: 12140, a primer consisting of the adaptor sequence iii) and the ORF specific sequence SEQ ID NO: 12336 and a second primer consisting of the adaptor sequence iiii) and the ORF specific sequence SEQ ID NO: 12337 were used.

For amplification and cloning of Arabidopsis thaliana SEQ ID NO: 1815, a primer consisting of the adaptor sequence iii) and the ORF specific sequence SEQ ID NO: 2361 and a second primer consisting of the adaptor sequence iiii) and the ORF specific sequence SEQ ID NO: 2362 were used.

For amplification and cloning of Azotobacter vinelandii SEQ ID NO: 6510, a primer consisting of the adaptor sequence iii) and the ORF specific sequence SEQ ID NO: 6670 and a second primer consisting of the adaptor sequence iiii) and the ORF specific sequence SEQ ID NO: 6671 were used.

For amplification and cloning of Brassica napus SEQ ID NO: 15532, a primer consisting of the adaptor sequence iii) and the ORF specific sequence SEQ ID NO: 16150 and a second primer consisting of the adaptor sequence iiii) and the ORF specific sequence SEQ ID NO: 16151 were used.

For amplification and cloning of Glycine max SEQ ID NO: 10811, a primer consisting of the adaptor sequence iii) and the ORF specific sequence SEQ ID NO: 11207 and a second primer consisting of the adaptor sequence iiii) and the ORF specific sequence SEQ ID NO: 11208 were used.

For amplification and cloning of Physcomytrella patens SEQ ID NO: 45757, a primer consisting of the adaptor sequence iii) and the ORF specific sequence SEQ ID NO: 45791 and a second primer consisting of the adaptor sequence iiii) and the ORF specific sequence SEQ ID NO: 45792 were used.

For amplification and cloning of Thermus thermophilus SEQ ID NO: 12698, a primer consisting of the adaptor sequence iii) and the ORF specific sequence SEQ ID NO: 12970 and a second primer consisting of the adaptor sequence iiii) and the ORF specific sequence SEQ ID NO: 12971 were used.

Following these examples every sequence disclosed in Table I, preferably column 5, especially the coding region thereof can be cloned by fusing the adaptor sequences to the respective specific primers sequences as disclosed in Table III, column 8 using the respective vectors shown in Table c. Table c showing binary vectors used in Example 11

TABLE c
Overview of the different vectors used for cloning the ORFs; showing their
SEQ ID NOs (column A), their vector names (column B), the promoters they
contain for expression of the ORFs (column C), if present, the additional artificial
targeting sequence (column D), the adapter sequence (column E), the expression
type conferred by the promoter mentioned in column B (column F)
and the figure number (column G).

| | B | C | D | E | | |
|---|---|---|---|---|---|---|
| A | Vector | Promoter | Target | Adapter | F | G |
| Seq ID | Name | Name | Sequence | Sequence | Expression Type | FIG. |
| 30 | pMTX0270p | Super | | Colic | non targeted constitutive | 6 |
| | | | | | expression preferentially | |
| | | | | | in green tissues | |
| 31 | pMTX155 | Big35S | | Resgen | non targeted constitutive | 7 |
| | | | | | expression preferentially | |
| | | | | | in green tissues | |
| 32 | VC- | Super | FNR | Resgen | plastidic targeted constitutive | 3 |
| | MME354- | | | | expression preferentially | |
| | 1QCZ | | | | in green tissues | |
| 34 | VC- | Super | IVD | Resgen | mitochondric targeted | 8 |
| | MME356- | | | | constitutive expression | |
| | 1QCZ | | | | preferentially in green | |
| | | | | | tissues | |
| 36 | VC- | USP | | Resgen | non targeted expression | 9 |
| | MME301- | | | | preferentially in seeds | |
| | 1QCZ | | | | | |
| 37 | pMTX461korrp | USP | FNR | Resgen | plastidic targeted expression | 10 |
| | | | | | preferentially in | |
| | | | | | seeds | |
| 39 | VC- | USP | IVD | Resgen | mitochondric targeted | 11 |
| | MME462- | | | | expression preferentially | |
| | 1QCZ | | | | in seeds | |
| 41 | VC- | Super | | Colic | non targeted constitutive | 1 |
| | MME220- | | | | expression preferentially | |
| | 1qcz | | | | in green tissues | |
| 42 | VC- | Super | FNR | Colic | plastidic targeted constitutive | 4 |
| | MME432- | | | | expression preferentially | |
| | 1qcz | | | | in green tissues | |
| 44 | VC- | Super | IVD | Colic | mitochondric targeted | 12 |
| | MME431- | | | | constitutive expression | |
| | 1qcz | | | | preferentially in green | |
| | | | | | tissues | |
| 46 | VC- | PcUbi | | Colic | non targeted constitutive | 2 |
| | MME221- | | | | expression preferentially | |
| | 1qcz | | | | in green tissues | |
| 47 | pMTX447korr | PcUbi | FNR | Colic | plastidic targeted constitutive | 13 |
| | | | | | expression preferentially | |
| | | | | | in green tissues | |
| 49 | VC- | PcUbi | IVD | Colic | mitochondric targeted | 14 |
| | MME445- | | | | constitutive expression | |
| | 1qcz | | | | preferentially in green | |
| | | | | | tissues | |
| 51 | VC- | USP | | Colic | non targeted expression | 15 |
| | MME289- | | | | preferentially in seeds | |
| | 1qcz | | | | | |
| 52 | VC- | USP | FNR | Colic | plastidic targeted expression | 16 |
| | MME464- | | | | preferentially in | |
| | 1qcz | | | | seeds | |
| 54 | VC- | USP | IVD | Colic | mitochondric targeted | 17 |
| | MME465- | | | | expression in preferentially | |
| | 1qcz | | | | seeds | |
| 56 | VC- | Super | | Resgen | non targeted constitutive | 5 |
| | MME489- | | | | expression preferentially | |
| | 1QCZ | | | | in green tissues | |

In column C PcUbi refers to the PcUbi promoter (Kawalleck et al., Plant. Molecular Biology, 21, 673 (1993)) also named p-PcUBI in table d, Super to the Super promoter (Ni et al,. Plant Journal 7, 661 (1995), WO 95/14098) also named p-Super in table d, Big35S to the enhanced 35S promoter (Comai et al., Plant Mol Biol 15, 373-383 (1990) and USP to the USP promoter (Bumlein et al., Mol Gen Genet. 225(3):459-67 (1991)) als named p-USP in table d.

### Example 11 b

### Construction of Binary Vectors for Non-Targeted Expression of Proteins

“Non-targeted” expression in this context means, that no additional targeting sequences were added to the ORF to be expressed.

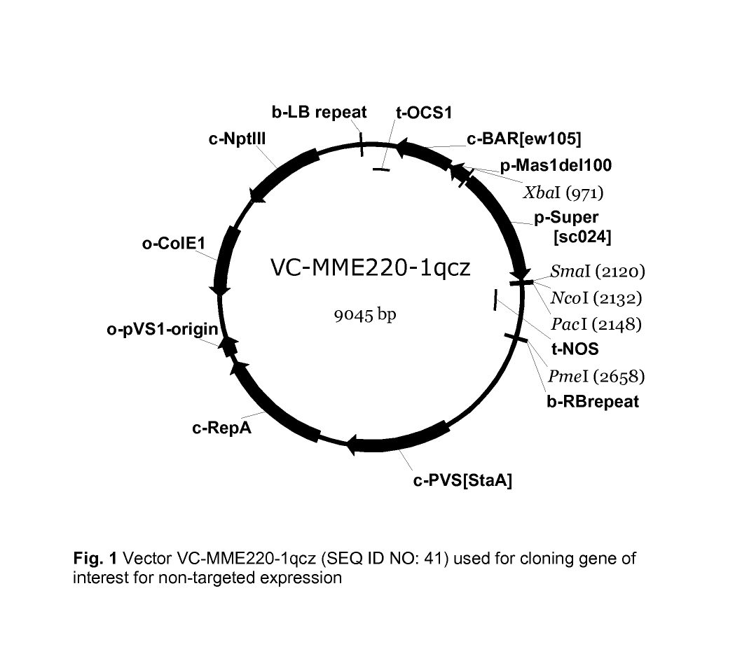

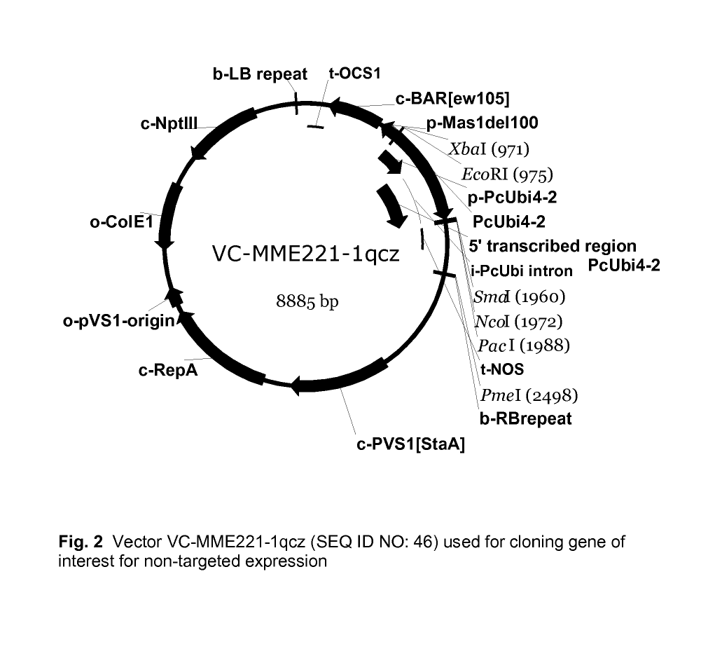

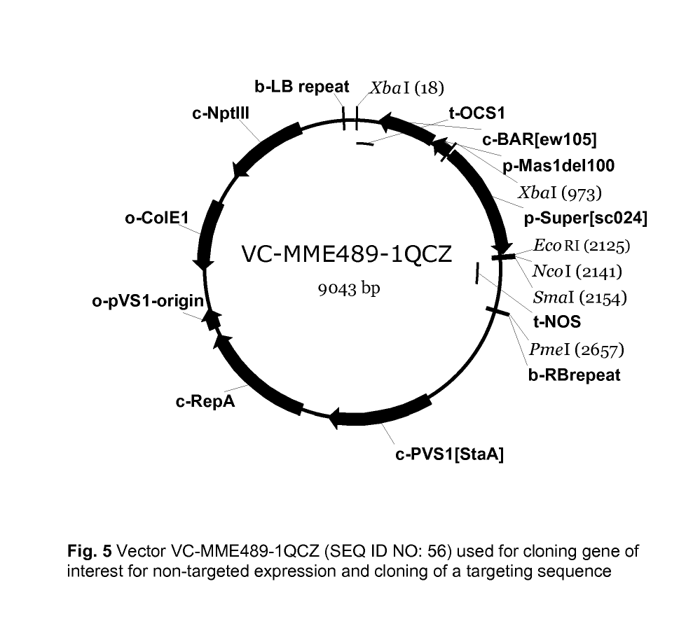

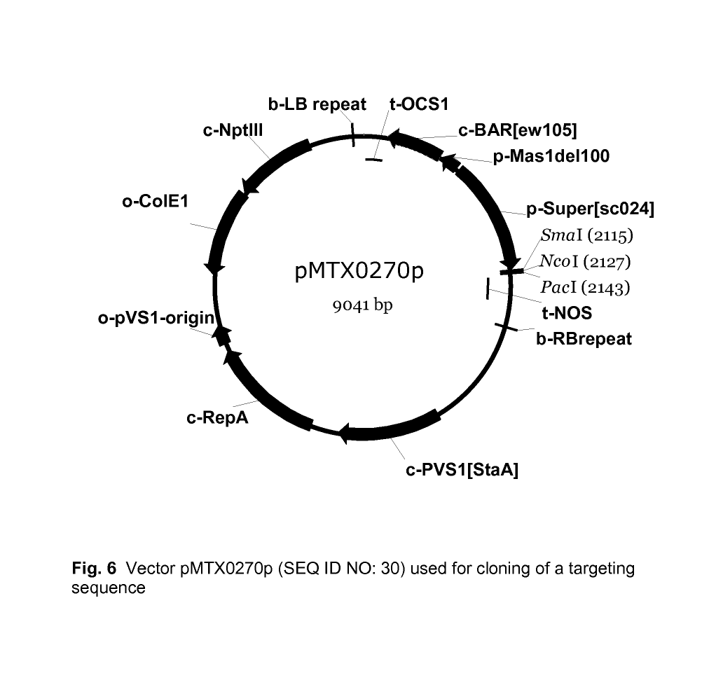

For non-targeted expression the binary vectors used for cloning were VC-MME220-1 qcz SEQ ID NO 41 (FIG. 1), VC-MME221-1qcz SEQ ID NO 46 (FIG. 2), and VC-MME489-1QCZ SEQ ID NO: 56 (FIG. 5), respectively. The binary vectors used for cloning the targeting sequence were VC-MME489-1QCZ SEQ ID NO: 56 (FIG. 5) and pMTX0270p SEQ ID NO 30 (FIG. 6), respectively. Other useful binary vectors are known to the skilled worker; an overview of binary vectors and their use can be found in Hellens R., Mullineaux P. and Klee H. (Trends in Plant Science, 5 (10), 446 (2000)). Such vectors have to be equally equipped with appropriate promoters and targeting sequences.

### Example 11c

### Construction of Binary Vectors for Plastidic-Targeted Expression of Proteins

For construction of a vector for plastid-targeted expression in preferential green tissues or preferential in seeds, the plastidic targeting sequence of the gene FNR from Spinacia oleracea was amplified. In order to amplify the targeting sequence, genomic DNA was extracted from leaves of 4 weeks old S. oleracea plants (DNeasy Plant Mini Kit, Qiagen, Hilden). The gDNA was used as the template for a PCR.

To enable cloning of the transit sequence into the vector VC-MME489-1QCZ and VC-MME301 1QCZ an EcoRl restriction enzyme recognition sequence was added to both the forward and reverse primers, whereas for cloning in the vectors pMTX0270p, VC-MME220-1 qcz, VCMME 221-1qcz and VC-MME289-1qcz a Pmel restriction enzyme recognition sequence was added to the forward primer and a Ncol site was added to the reverse primer.

| | |
|---|---|
| FNR5EcoResgen | ATA GAA TTC GCA TAA ACT TAT CTT CAT AGT TGC C |
| | SEQ ID NO: 24 |
| FNR3EcoResgen | ATA GAA TTC AGA GGC GAT CTG GGC CCT |
| | SEQ ID NO: 25 |
| FNR5PmeColic | ATA GTT TAA ACG CAT AAA CTT ATC TTC ATA GTT GCC |
| | SEQ ID NO: 26 |
| FNR3NcoColic | ATA CCA TGG AAG AGC AAG AGG CGA TCT GGG CCC T |
| | SEQ ID NO: 27 |

The resulting sequence SEQ ID NO: 28 amplified from genomic spinach DNA, comprised a 511TR (bp 1-165), and the coding region (bp 166-273 and 351-419). The coding sequence is interrupted by an intronic sequence from by 274 to by 350:

(SEQ ID NO: 28)
gcataaacttatcttcatagttgccactccaatttgctccttgaatctcctccacccaatacataatccac
tcctccatcacccacttcactactaaatcaaacttaactctgtttttctctctcctcctttcatttcttat
tcttccaatcatcgtactccgccatgaccaccgctgtcaccgccgctgtttctttcccctctaccaaaacc
acctctctctccgcccgaagctcctccgtcatttcccctgacaaaatcagctacaaaaaggtgattcccaa
tttcactgtgttttttattaataatttgttattttgatgatgagatgattaatttgggtgctgcaggttcc
tttgtactacaggaatgtatctgcaactgggaaaatgggacccatcagggcccagatcgcctct

The PCR fragment derived with the primers FNR5EcoResgen and FNR3EcoResgen was digested with EcoRI and ligated in the vectors VC-MME489-1QCZ and VC-MME301-1QCZ, that had also been digested with EcoRl. The correct orientation of the FNR targeting sequence was tested by sequencing. The vector generated in this ligation step were VC-MME354-1 QCZ and pMTX461 korrp, respectively.

The PCR fragment derived with the primers FNR5PmeColic and FNR3NcoColic was digested with Pmel and Ncol and ligated in the vectors pMTX0270p, VC-MME220-1qcz, VC-MME221 1qcz and VC-MME289-1qcz that had been digested with Smal and Ncol. The vectors generated in this ligation step were VC-MME432-1 qcz, VC-MME464-1 qcz and pMTX447korr, respectively.

For plastidic-targeted constitutive expression in preferentially green tissues an artifical promoter A(ocs)3AmasPmas promoter (Super promoter)) (Ni et al, Plant Journal 7, 661 (1995), WO 95/14098) was used in context of the vector VC-MME354-1QCZ for ORFs from Saccharomyces cerevisiae as well as Arabidopsis thaliana and in context of the vector VC-MME432-1 qcz for ORFs from Escherichia coli as well as Arabidopsis thaliana, resulting in each case in an “inframe” fusion of the FNR targeting sequence with the ORFs.

For plastidic-targeted expression in preferentially seeds the USP promoter (Baumlein et al., Mol Gen Genet. 225(3):459-67 (1991)) was used in context of either the vector pMTX461 korrp for ORFs from Saccharomyces cerevisiae or in context of the vector VC-MME464-1 qcz for ORFs from Escherichia coli, resulting in each case in an “in-frame” fusion of the FNR targeting sequence with the ORFs.

For plastidic-targeted constitutive expression in preferentially green tissues and seeds the PcUbi promoter was used in context of the vector pMTX447korr for ORFs from Saccharomyces cerevisiae, Escherichia coli, Synechocystis sp., Azotobacter vinelandii, Thermus thermophilus, Arabidopsis thaliana, Brassica napus, Glycine max, Oryza sativa, Physcomitrella patens, or Zea mays, resulting in each case in an “in-frame” fusion of the FNR targeting sequence with the ORFs.

### Example 11d

### Construction of Binary Vectors for Mitochondric-Targeted Expression of Proteins

Amplification of the mitochondrial targeting sequence of the gene IVD from Arabidopsis thaliana and construction of vector for mitochondrial-targeted expression in preferential green tissues or preferential in seeds.

In order to amplify the targeting sequence of the IVD gene from A. thaliana, genomic DNA was extracted from leaves of A. thaliana plants (DNeasy Plant Mini Kit, Qiagen, Hilden). The gDNA was used as the template for a PCR.

To enable cloning of the transit sequence into the vectors VC-MME489-1 QCZ and VC-MME-301-1QCZ an EcoRI restriction enzyme recognition sequence was added to both the forward and reverse primers, whereas for cloning in the vectors VC-MME220-1qcz, VC-MME-221-1qcz and VC-MME289-1qcz a Pmel restriction enzyme recognition sequence was added to the forward primer and a Ncol site was added to the reverse primer.

| | |
|---|---|
| IVD5EcoResgen | ATA GAA TTC ATG CAG AGG TTT TTC TCC GC |
| | SEQ ID NO: 57 |
| IVD3EcoResgen | ATA GAA TTC CGA AGA ACG AGA AGA GAA AG |
| | SEQ ID NO: 58 |
| IVD5PmeColic | ATA GTT TAA ACA TGC AGA GGT TTT TCT CCG C |
| | SEQ ID NO: 59 |
| IVD3NcoColic | ATA CCA TGG AAG AGC AAA GGA GAG ACG AAG AAC GAG |
| | SEQ ID NO: 60 |

The resulting sequence (SEQ ID NO: 61) amplified from genomic A. thaliana DNA with IVD5EcoResgen and IVD3EcoResgen comprised 81 bp:

(SEQ ID NO: 61)
atgcagaggtttttctccgccagatcgattctcggttacgccgtcaag
acgcggaggaggtctttctcttctcgttcttcg

The resulting sequence (SEQ ID NO: 62) amplified from genomic A. thaliana DNA with IVD5PmeColic and IVD3NcoColic comprised 89 bp:

(SEQ ID NO: 62)
atgcagaggtttttctccgccagatcgattctcggttacgcc
gtcaagacgcggaggaggtctttctcttctcgttcttcgtctctcct

The PCR fragment derived with the primers IVD5EcoResgen and IVD3EcoResgen was digested with EcoRl and ligated in the vectors VC-MME489-1QCZ and VC-MME301-1QCZ that had also been digested with EcoRl. The correct orientation of the IVD targeting sequence was tested by sequencing. The vectors generated in this ligation step were VC-MME356-1 QCZ and VC-MME462-1 QCZ, respectively.

The PCR fragment derived with the primers IVD5PmeColic and IVD3NcoColic was digested with Pmel and Ncol and ligated in the vectors VC-MME220-1qcz, VC-MME221-1qcz and VC-MME289-1qcz that had been digested with Smal and Ncol. The vectors generated in this ligation step were VC-MME431-1qcz, VC-MME465-1qcz and VC-MME445-1qcz, respectively.

For mitochondrial-targeted constitutive expression in preferentially green tissues an artificial promoter A(ocs)3AmasPmas promoter (Super promoter) (Ni et al, Plant Journal 7, 661 (1995), WO 95/14098) was used in context of the vector VC-MME356-1 QCZ for ORFs from Saccharomyces cerevisiae and in context of the vector VC-MME431-1qcz for ORFs from Escherichia coli, resulting in each case in an “in-frame” fusion between the IVD sequence and the respective ORFs.

For mitochondrial-targeted constitutive expression in preferentially seeds the USP promoter (Bäumlein et al., Mol Gen Genet. 225 (3), 459 (1991)) was used in context of the vector VC-MME462-1QCZ for ORFs from Saccharomyces cerevisiae and in context of the vector VC-MME465-1qcz for ORFs from Escherichia coli, resulting in each case in an “in-frame” fusion between the IVD sequence and the respective ORFs.

For mitochondrial-targeted constitutive expression in preferentially green tissues and seeds the

PcUbi promoter was used in context of the vector VC-MME445-1 qcz for ORFs from Saccharomyces cerevisiae, Escherichia coli, Synechocystis sp., Azotobacter vinelandii, Thermus thermophilus, Arabidopsis thaliana, Brassica napus, Glycine max, Oryza sativa, Physcomitrella patens, or Zea mays, resulting in each case in an “in-frame” fusion between the IVD sequence and the respective ORFs.

Other useful binary vectors are known to the skilled worker; an overview of binary vectors and their use can be found in Hellens R., Mullineaux P. and Klee H., (Trends in Plant Science, 5 (10), 446 (2000)). Such vectors have to be equally equipped with appropriate promoters and targeting sequences.

### Example 11e

### Cloning of Inventive Sequences as Shown in Table I, Column 5 in the Different Expression Vectors

For cloning of for example SEQ ID NO: 7081 from Escherichia coli or any other ORF from Escherichia coli, Synechocystis sp., Azotobacter vinelandii, Thermus thermophilus, Arabidopsis thaliana, Brassica napus, Glycine max or Physcomitrella patens the vector DNA was treated with the restriction enzymes Pacl and Ncol following the standard protocol (MBI Fermentas).

For cloning for example the ORFs of SEQ ID NO: 113598 from Saccharomyces cerevisiae or any other ORF from Saccharomyces cerevisiae into vectors containing the Resgen adaptor sequence the respective vector DNA was treated with the restriction enzyme Ncol.

For cloning of for example ORFs SEQ ID NO: 66274 from Saccharomyces cerevisiae or any other ORF from Saccharomyces cerevisiae into vectors containing the Colic adaptor sequence, the respective vector DNA was treated with the restriction enzymes Pacl and Ncol following the standard protocol (MBI Fermentas).

In all cases the reaction was stopped by inactivation at 70° C. for 20 minutes and purified over QlAquick or NucleoSpin Extract II columns following the standard protocol (Qiagen or Macherey-Nagel).

Then the PCR-product representing the amplified ORF with the respective adapter sequences and the vector DNA were treated with T4 DNA polymerase according to the standard protocol (MBI Fermentas) to produce single stranded overhangs with the parameters 1 unit T4 DNA polymerase at 37° C. for 2-10 minutes for the vector and 1-2 u T4 DNA polymerase at 15-17° C. for 10-60 minutes for the PCR product representing SEQ ID NO: 7081.

The reaction was stopped by addition of high-salt buffer and purified over QlAquick or NucleoSpin Extract II columns following the standard protocol (Qiagen or Macherey-Nagel).

According to this example the skilled person is able to clone all sequences disclosed in Table I, preferably column 5 or column 8, especially the coding region thereof.

Approximately 30-60 ng of prepared vector and a defined amount of prepared amplificate were mixed and hybridized at 65° C. for 15 minutes followed by 37° C. 0.1° C./1 seconds, followed by 37° C. 10 minutes, followed by 0.1° C./1 seconds, then 4-10° C.

The ligated constructs were transformed in the same reaction vessel by addition of competent E. coli cells (strain DHSalpha) and incubation for 20 minutes at 1° C. followed by a heat shock for 90 seconds at 42° C. and cooling to 1-4° C. Then, complete medium (SOC) was added and the mixture was incubated for 45 minutes at 37° C. The entire mixture was subsequently plated onto an agar plate with 0.05 mg/ml kanamycin and incubated overnight at 37° C.

The outcome of the cloning step was verified by amplification with the aid of primers which bind upstream and downstream of the integration site, thus allowing the amplification of the insertion.

The amplifications were carried out as described in the protocol of Taq DNA polymerase (Gibco-BRL).

The amplification cycles were as follows: 1 cycle of 1-5 minutes at 94° C., followed by 35 cycles of in each case 15-60 seconds at 94° C., 15-60 seconds at 50-66° C. and 5-15 minutes at 72° C., followed by 1 cycle of 10 minutes at 72° C., then 4-16° C. Several colonies were checked, but only one colony for which a PCR product of the expected size was detected was used in the following steps.

A portion of this positive colony was transferred into a reaction vessel filled with complete medium (LB) supplemented with kanamycin and incubated overnight at 37° C.

The plasmid preparation was carried out as specified in the Qiaprep or NucleoSpin Multi-96 Plus standard protocol (Qiagen or Macherey-Nagel).

### Example 11f

### Generation of Transgenic Plants which Express Sequences as Shown in Table I, Preferably Column 5

1-5 ng of the plasmid DNA isolated was transformed by electroporation or transformation into competent cells of Agrobacterium tumefaciens, of strain GV 3101 pMP90 (Koncz and Schell, Mol. Gen. Gent. 204, 383 (1986)). Thereafter, complete medium (YEP) was added and the mixture was transferred into a fresh reaction vessel for 3 hours at 28° C. Thereafter, all of the reaction mixture was plated onto YEP agar plates supplemented with the respective antibiotics, e.g. rifampicine (0.1 mg/ml), gentamycine (0.025 mg/ml and kanamycin (0.05 mg/ml) and incubated for 48 hours at 28° C.

The agrobacteria that contains the plasmid construct were then used for the transformation of plants.

A colony was picked from the agar plate with the aid of a pipette tip and taken up in 3 ml of liquid TB medium, which also contained suitable antibiotics as described above. The preculture was grown for 48 hours at 28° C. and 120 rpm.

400 ml of LB medium containing the same antibiotics as above were used for the main culture. The preculture was transferred into the main culture. It was grown for 18 hours at 28° C. and 120 rpm. After centrifugation at 4 000 rpm, the pellet was resuspended in infiltration medium (MS medium, 10% sucrose).

In order to grow the plants for the transformation, dishes (Piki Saat 80, green, provided with a screen bottom, 30×20×4.5 cm, from Wiesauplast, Kunststofftechnik, Germany) were half-filled with a GS 90 substrate (standard soil, Werkverband E.V., Germany). The dishes were watered overnight with 0.05% Proplant solution (Chimac-Apriphar, Belgium). A. thaliana C24 seeds (Nottingham Arabidopsis Stock Centre, UK; NASC Stock N906) were scattered over the dish, approximately 1 000 seeds per dish. The dishes were covered with a hood and placed in the stratification facility (8 h, 110 μmol m⁻²s⁻¹, 22° C.; 16 h, dark, 6° C.). After 5 days, the dishes were placed into the short-day controlled environment chamber (8 h, 130 μmol m⁻²s⁻¹, 22° C.; 16 h, dark, 20° C.), where they remained for approximately 10 days until the first true leaves had formed.

The seedlings were transferred into pots containing the same substrate (Teku pots, 7 cm, LC series, manufactured by Poppelmann GmbH & Co, Germany). Five plants were pricked out into each pot. The pots were then returned into the short-day controlled environment chamber for the plant to continue growing.

After 10 days, the plants were transferred into the greenhouse cabinet (supplementary illumination, 16 h, 340 μmol m⁻²s⁻¹, 22° C.; 8 h, dark, 20° C.), where they were allowed to grow for further 17 days.

For the transformation, 6-week-old Arabidopsis plants, which had just started flowering were immersed for 10 seconds into the above-described agrobacterial suspension which had previously been treated with 10 μl Silwett L77 (Crompton S. A., Osi Specialties, Switzerland). The method in question is described by Clough J. C. and Bent A. F. (Plant J. 16, 735 (1998)).

The plants were subsequently placed for 18 hours into a humid chamber. Thereafter, the pots were returned to the greenhouse for the plants to continue growing. The plants remained in the greenhouse for another 10 weeks until the seeds were ready for harvesting.

Depending on the tolerance marker used for the selection of the transformed plants the harvested seeds were planted in the greenhouse and subjected to a spray selection or else first sterilized and then grown on agar plates supplemented with the respective selection agent. Since the vector contained the bar gene as the tolerance marker, plantlets were sprayed four times at an interval of 2 to 3 days with 0.02% BASTA® and transformed plants were allowed to set seeds.

The seeds of the transgenic A. thaliana plants were stored in the freezer (at −20° C.).

### Example 11g

### Plant Culture (Arabidopsis) for Bioanalytical Analyses

For the bioanalytical analyses of the transgenic plants, the latter were grown uniformly a specific culture facility. To this end the GS-90 substrate as the compost mixture was introduced into the potting machine (Laible System GmbH, Singen, Germany) and filled into the pots. Thereafter, 35 pots were combined in one dish and treated with Previcur. For the treatment, 25 ml of Previcur were taken up in 10 l of tap water. This amount was sufficient for the treatment of approximately 200 pots. The pots were placed into the Previcur solution and additionally irrigated overhead with tap water without Previcur. They were used within four days.

For the sowing, the seeds, which had been stored in the refrigerator (at −20° C.), were removed from the Eppendorf tubes with the aid of a toothpick and transferred into the pots with the compost. In total, approximately 5 to 12 seeds were distributed in the middle of the pot.

After the seeds had been sown, the dishes with the pots were covered with matching plastic hood and placed into the stratification chamber for 4 days in the dark at 4° C. The humidity was approximately 90%. After the stratification, the test plants were grown for 22 to 23 days at a 16-h-light, 8-h-dark rhythm at 20° C., an atmospheric humidity of 60% and a CO2 concentration of approximately 400 ppm. The light sources used were Powerstar HQI-T 250 W/D Daylight lamps from Osram, which generate a light resembling the solar color spectrum with a light intensity of approximately 220 E/m2/s−1.

Selection of transgenic plants was depending on the use resistance marker. In case of the bar gene as the resistance marker plantlets were sprayed three times at days 8-10 after sowing with 0.02% BASTA®, (Glufosinate ammonium;) Bayer CropScience, Germany, Leverkusen .The resistance plants were thinned when they had reached the age of 14 days. The plants, which had grown best in the center of the pot were considered the target plants. All the remaining plants were removed carefully with the aid of metal tweezers and discarded.

During their growth, the plants received overhead irrigation with distilled water (onto the compost) and bottom irrigation into the placement grooves. Once the grown plants had reached the age of 23 days, they were harvested. In case their seeds are desired these had been harvested 10 to 12 weeks after sowing (once they are ripe).

### Example 11h

### Metabolic Analysis of Transformed Plants

The modifications identified in accordance with the invention, in the content of above-described metabolites, were identified by the following procedure.

a) sampling and Storage of the Samples

Sampling was performed directly in the controlled-environment chamber. The plants, or respective parts thereof, like leafs, were cut using small laboratory scissors, rapidly weighed on laboratory scales, transferred into a pre-cooled extraction thimble and placed into an aluminum rack cooled by liquid nitrogen. If required, the extraction thimbles can be stored in the freezer at −80° C. The time elapsing between cutting the plant/plant parts to freezing it in liquid nitrogen amounted to not more than 10 to 20 seconds.

### b) Lyophilization

During the experiment, care was taken that the plants either remained in the deep-frozen state (temperatures <−40° C.) or were freed from water by lyophilization until the first contact with solvents.

The aluminum rack with the plant samples in the extraction thimbles was placed into the precooled (−40° C.) lyophilization facility. The initial temperature during the main drying phase was −35° C. and the pressure was 0.120 mbar. During the drying phase, the parameters were altered following a pressure and temperature program. The final temperature after 12 hours was +30° C. and the final pressure was 0.001 to 0.004 mbar. After the vacuum pump and the refrigerating machine had been switched off, the system was flushed with air (dried via a drying tube) or argon.

### c) Extraction

### Extraction of Arabidopsis Green Tissue:

Immediately after the lyophilization apparatus had been flushed, the extraction thimbles with the lyophilized plant material were transferred into the 5 ml extraction cartridges of the ASE device (Accelerated Solvent Extractor ASE 200 with Solvent Controller and AutoASE software (DIONEX)).

The 24 sample positions of an ASE device (Accelerated Solvent Extractor ASE 200 with Solvent

Controller and AutoASE software (DIONEX)) were filled with plant samples, including some samples for testing quality control.

The polar substances were extracted with approximately 10 ml of methanol/water (80/20, v/v) at T=70° C. and p=140 bar, 5 minutes heating-up phase, 1 minute static extraction. The more lipophilic substances were extracted with approximately 10 ml of methanol/dichloromethane (40/60, v/v) at T=70° C. and p=140 bar, 5 minute heating-up phase, 1 minute static extraction. The two solvent mixtures were extracted into the same glass tubes (centrifuge tubes, 50 ml, equipped with screw cap and pierceable septum for the ASE (DIONEX)).

The solution was treated with commercial available internal standards, such as ribitol, L-glycine-2,2-d2, L-alanine-2,3,3,3-d4, methionine-d3, Arginine_(13C), Tryptophan-d5, and α-methylglucopyranoside and methyl nonadecanoate, methyl undecanoate, methyl tridecanoate, methyl pentadecanoate, methyl nonacosanoate

The total extract was treated with 8 ml of water. The solid residue of the plant sample and the extraction thimbles were discarded.

The extract was shaken and then centrifuged for 5 to 10 minutes at at least 1400 g in order to accelerate phase separation. 1 ml of the supernatant methanol/water phase (“polar phase”, colorless) was removed for the further GC analysis, and 1 ml was removed for the LC analysis. The remainder of the methanol/water phase was discarded. 0.5 ml of the organic phase (“lipid phase”, dark green) was removed for the further GC analysis and 0.5 ml was removed for the LC analysis. All the portions removed were evaporated to dryness using the IR Dancer infrared vacuum evaporator (Hettich). The maximum temperature during the evaporation process did not exceed 40° C. Pressure in the apparatus was not less than 10 mbar.

Extraction of Arabidopsis Seeds:

3 mg of Arabidopsis seeds are transferred into a 1.2-mL-stainless steel grinding jar and ground and extracted with a mixture of 770 μL methanol and 290 μL water. A solution containing commercially available standard substances (ribitol, L-glycine-2,2-d2, L-alanine-2,3,3,3-d4, methionine-methyl-d3, tryptophane-d5, Arginine 13C615N4, Pep3 (Boc-Ala-Gly-Gly-Gly-OH) and amethylglucopyranoside) is added as internal standard. The extraction is performed using a stainless steel ball and a ball mill (Retsch MM 200, Retsch, Germany) operated at 30 Hz for 3 minutes. After centrifugation at 6000 rpm for 5 min 800 μL of the extraction solvent is transferred into a 2-mL-reaction tube (Eppendorf).

A solution of commercially available internal standard substances (Coenzyme Q1, Coenzyme Q2, Coenzyme Q4, and methyl nonadecanoate, undecanoic acid, tridecanoic acid, pentadecanoic acid, methyl nonacosanoate) is added as internal standard. For the extraction of lipophilic metabolites, 640 μL methylene chloride and 170 μL methanol are added and the sample is extracted in a ball mill operated at 30 Hz for 3 minutes. After centrifugation at 6000 rpm for 5 min 800 μL of the extraction solvent is transferred and combined with the extract of the first extraction step. After the addition of 400 μL of water and a centrifugation step to ensure proper separation of the organic and aqueous layer, two aliquots of 500 μL of the aqueous top layer (polar phase) are taken for GC and LC analysis, respectively.

Two aliquots of 100 μL of the organic bottom layer (lipid phase) are take for GC and LC analysis, respectively.

All the portions removed were evaporated to dryness using the IR Dancer infrared vacuum evaporator (Hettich). The maximum temperature during the evaporation process did not exceed 40° C. Pressure in the apparatus was not less than 10 mbar.

### Extraction of Rice or Corn Seed Material:

20 rice or corn kernels are homogenized with a 50-mL-stainless steel grinding jar and ground with a stainless steel grinding ball using a ball mill (Retsch MM 200, Retsch, Germany) operated at 30 Hz for 3 minutes. The ground samples are lyophilized over night The initial temperature during the main drying phase was −35° C. and the pressure was 0.120 mbar. During the drying phase, the parameters were altered following a pressure and temperature program. The final temperature after 12 hours was +30° C. and the final pressure was 0.001 to 0.004 mbar. After the vacuum pump and the refrigerating machine had been switched off, the system was flushed with air (dried via a drying tube) or argon.

50 mg of the lyophilized kernel material are weighed into glass fibre extraction thimbles and extracted and further processed as described for the Extraction of Arabidopsis green tissue.

d) Processing the Lipid and Polar Phase for the LC/MS or LC/MS/MS Analysis The lipid extract, which had been evaporated to dryness was taken up in mobile phase. The polar extract, which had been evaporated to dryness was taken up in mobile phase.

### LC-MS Analysis:

The LC part was carried out on a commercially available LCMS system from Agilent Technologies, USA. For polar extracts 10 μl are injected into the system at a flow rate of 200μl/min. The separation column (Reversed Phase C18) was maintained at 15° C. during chromatography. For lipid extracts 5 pl are injected into the system at a flow rate of 200 μl/min. The separation column (Reversed Phase C18) was maintained at 30° C. HPLC was performed with gradient elution.

The mass spectrometric analysis was performed on an Applied Biosystems API 4000 triple quadrupole instrument with turbo ion spray source. For polar extracts the instrument measured in negative ion mode in MRM-mode and fullscan mode from 100-1000 amu. For lipid extracts the instrument measured in positive ion mode in MRM-mode fullscan mode from 100-1000 amu. MS analysis is described in more detail in patent publication number WO 03/073464 (Walk and Dostler).

### e) Derivatization of the Lipid and Polar Phase for the GC/MS Analysis

Derivatization of the lipid phase for the GC/MS analysis: For the transmethanolysis, a mixture of 140 μl of chloroform, 37 μl of hydrochloric acid (37% by weight HCl in water), 320 μl of methanol and 20 μl of toluene was added to the evaporated extract of the lipid phase. The vessel was sealed tightly and heated for 2 hours at 100° C., with shaking. The solution was subsequently evaporated to dryness. The residue was dried completely.

The methoximation of the carbonyl groups was carried out by reaction with methoxyamine hydrochloride (5 mg/ml in pyridine, 100 μl for 1.5 hours at 60° C.) in a tightly sealed vessel. 20 μl of a solution of odd-numbered, straight-chain fatty acids (solution of each 0.3 mg/mL of fatty acids from 7 to 25 carbon atoms and each 0.6 mg/mL of fatty acids with 27, 29 and 31 carbon atoms in 3/7 (v/v) pyridine/toluene) were added as time standards. Finally, the derivatization with 100 μl of N-methyl-N-(trimethylsilyl)-2,2,2-trifluoroacetamide (MSTFA) was carried out for 30 minutes at 60° C., again in the tightly sealed vessel. The final volume before injection into the GC was 220 pl.

### Derivatization of the Polar Phase for the GC/MS Analysis:

The methoximation of the carbonyl groups was carried out by reaction with methoxyamine hydrochloride (5 mg/ml in pyridine, 50 pl for 1.5 hours at 60° C.) in a tightly sealed vessel. 10 pl of a solution of odd-numbered, straight-chain fatty acids (solution of each 0.3 mg/mL of fatty acids from 7 to 25 carbon atoms and each 0.6 mg/mL of fatty acids with 27, 29 and 31 carbon atoms in 3/7 (v/v) pyridine/toluene) were added as time standards. Finally, the derivatization with 50 pl of N-methyl-N-(trimethylsilyl)-2,2,2-trifluoroacetamide (MSTFA) was carried out for 30 minutes at 60° C., again in the tightly sealed vessel. The final volume before injection into the GC was 110 μl.

### f) GC-MS Analysis

The GC-MS systems consisted of an Agilent 6890 GC coupled to an Agilent 5973 MSD. The autosamplers were CompiPal or GCPal from CTC. For the analysis usual commercial capillary separation columns (30 m×0.25 mm×0.25 μm) with different poly-methyl-siloxane stationary phases containing 0% up to 35% of aromatic moieties, depending on the analysed sample materials and fractions from the phase separation step, were used (for example: DB-1ms, HP-5ms, DB-XLB, DB-35ms, Agilent Technologies). Up to 1 μL of the final volume was injected splitless and the oven temperature program was started at 70° C. and ended at 340° C. with different heating rates depending on the sample material and fraction from the phase separation step in order to achieve a sufficient chromatographic separation and number of scans within each analyte peak. Usual GC-MS standard conditions, for example constant flow with nominal 1 to 1.7 ml/min. and helium as the mobile phase gas were used. Ionisation was done by electron impact with 70 eV, scanning within a m/z range from 15 to 600 with scan rates from 2.5 to 3 scans/sec and standard tune conditions.

### g) Analysis of the Various Plant Samples

The samples were measured in individual series of 20 to 21 plant or seed samples each (also referred to as sequences), each sequence containing at least 5 wild-type plants or seed samples as controls. Seed samples were from individual plants. The peak area of each analyte was divided by the peak area of the respective internal standard. The data were standardized for the fresh weight established for the plant or seed sample, respectively. The values calculated thus were related to the wild-type control group by being divided by the mean of the corresponding data of the wild-type control group of the same sequence. The values obtained were referred to as ratio_by_WT, they are comparable between sequences and indicate how much the analyte concentration in the mutant differs in relation to the wild-type control. Appropriate controls were done before to proof that the vector and transformation procedure itself has no significant influence on the metabolic composition of the plants. Therefore the described changes in comparison with wild types were caused by the introduced gene constructs. At least 3-5 independent lines were analyzed in two independent experiments for each construct.

The results of the different plant analyses can be seen from the Table d.

Table d showing results of plant analyses

TABLE d

| SeqID | Target | Locus | Metabolite | Source | Promotor | Method | Min | Max |
|---|---|---|---|---|---|---|---|---|
| 15532 | plastidic | 51340801_CANOLA | a-toco | ARA_LEAF | p-PcUBI | GC | 45 | 65 |
| 15532 | plastidic | 51340801_CANOLA | g-toco | ARA_LEAF | p-PcUBI | LC | 38 | 134 |
| 16883 | plastidic | 59582753_SOYBEAN | a-toco | ARA_LEAF | p-PcUBI | GC | 50 | 120 |
| 17901 | non-targ | At1g29350 | g-toco | ARA_LEAF | p-PcUBI | LC | 40 | 88 |
| 18070 | non-targ | At1g43850 | g-toco | ARA_LEAF | p-PcUBI | LC | 55 | 134 |
| 18235 | non-targ | At1g48260 | g-toco | ARA_LEAF | p-PcUBI | LC | 66 | 160 |
| 18869 | non-targ | At1g61950 | g-toco | ARA_LEAF | p-PcUBI | LC | 44 | 91 |
| 19419 | non-targ | At1g72770 | a-toco | ARA_LEAF | p-PcUBI | GC | 63 | 133 |
| 108727 | non-targ | At2g42830 | a-toco | ARA_LEAF | p-PcUBI | LC | 36 | 55 |
| 21159 | non-targ | At2g47880 | g-toco | ARA_LEAF | p-PcUBI | LC | 87 | 232 |
| 21497 | plastidic | At3g04050 | g-toco | ARA_LEAF | p-PcUBI | LC | 74 | 160 |
| 21902 | non-targ | At3g04710 | a-toco | ARA_LEAF | p-PcUBI | LC | 39 | 64 |
| 22015 | non-targ | At3g06270 | a-toco | ARA_LEAF | p-PcUBI | GC | 56 | 56 |
| 22249 | non-targ | At3g08710 | a-toco | ARA_LEAF | p-PcUBI | GC | 57 | 140 |
| 22611 | non-targ | At3g11650 | a-toco | ARA_LEAF | p-PcUBI | LC | 40 | 67 |
| 22611 | non-targ | At3g11650 | g-toco | ARA_LEAF | p-PcUBI | LC | 43 | 300 |
| 1815 | non-targ | At3g23000 | g-toco | ARA_LEAF | p-PcUBI | LC | 60 | 223 |
| 2573 | non-targ | At3g62930 | g-toco | ARA_LEAF | p-PcUBI | LC | 63 | 110 |
| 2935 | non-targ | At3g62950 | g-toco | ARA_LEAF | p-PcUBI | LC | 77 | 155 |
| 109717 | non-targ | At4g09960 | a-toco | ARA_LEAF | p-PcUBI | GC | 59 | 154 |
| 23482 | non-targ | At4g15660 | a-toco | ARA_LEAF | p-PcUBI | GC | 50 | 139 |
| 23482 | non-targ | At4g15660 | g-toco | ARA_LEAF | p-PcUBI | LC | 38 | 81 |
| 3279 | non-targ | At4g15670 | a-toco | ARA_LEAF | p-PcUBI | GC | 53 | 364 |
| 23844 | non-targ | At4g15690 | a-toco | ARA_LEAF | p-PcUBI | GC | 53 | 125 |
| 68849 | non-targ | At4g26080 | a-toco | ARA_LEAF | p-PcUBI | GC | 54 | 99 |
| 24311 | non-targ | At4g34160 | a-toco | ARA_LEAF | p-PcUBI | GC | 51 | 200 |
| 4348 | non-targ | At4g35310 | a-toco | ARA_LEAF | p-PcUBI | GC | 91 | 195 |
| 4348 | non-targ | At4g35310 | g-toco | ARA_LEAF | p-PcUBI | LC | 69 | 192 |
| 70006 | non-targ | At5g60110 | a-toco | ARA_LEAF | p-PcUBI | GC | 58 | 69 |
| 70006 | non-targ | At5g60110 | g-toco | ARA_LEAF | p-PcUBI | LC | 48 | 108 |
| 5493 | non-targ | At5g64920 | g-toco | ARA_LEAF | p-PcUBI | LC | 42 | 75 |
| 110651 | non-targ | Avin- | g-toco | ARA_LEAF | p-PcUBI | LC | 39 | 66 |
| | | DRAFT_3107 | | | | | | |
| 29286 | non-targ | Avin- | g-toco | ARA_LEAF | p-PcUBI | LC | 53 | 89 |
| | | DRAFT_3209 | | | | | | |
| 111155 | non-targ | Avin- | a-toco | ARA_LEAF | p-PcUBI | LC | 37 | 38 |
| | | DRAFT_3577 | | | | | | |
| 102941 | non-targ | Avin- | a-toco | ARA_LEAF | p-PcUBI | GC | 51 | 68 |
| | | DRAFT_4847 | | | | | | |
| 6510 | non-targ | Avin- | a-toco | ARA_LEAF | p-PcUBI | GC | 145 | 201 |
| | | DRAFT_5103 | | | | | | |
| 6510 | non-targ | Avin- | g-toco | ARA_LEAF | p-PcUBI | LC | 40 | 237 |
| | | DRAFT_5103 | | | | | | |
| 74729 | non-targ | Avin- | g-toco | ARA_LEAF | p-PcUBI | LC | 44 | 243 |
| | | DRAFT_6679 | | | | | | |
| 34889 | plastidic | B0004 | g-toco | ARA_LEAF | p-Super | LC | 75 | 443 |
| 35482 | non-targ | B0124 | a-toco | ARA_LEAF | p-Super | LC | 38 | 103 |
| 35482 | non-targ | B0124 | g-toco | ARA_LEAF | p-Super | LC | 175 | 277 |
| 7081 | non-targ | B0161 | g-toco | ARA_LEAF | p-Super | LC | 98 | 238 |
| 111340 | non-targ | B0421 | a-toco | ARA_LEAF | p-Super | LC | 42 | 44 |
| 7333 | non-targ | B0449 | g-toco | ARA_LEAF | p-Super | LC | 42 | 248 |
| 75286 | plastidic | B0754 | a-toco | ARA_LEAF | p-USP | LC | 35 | 68 |
| 7917 | non-targ | B0898 | g-toco | ARA_LEAF | p-Super | LC | 76 | 379 |
| 37394 | non-targ | B1163 | a-toco | ARA_LEAF | p-Super | GC | 52 | 56 |
| 111910 | non-targ | B1183 | g-toco | ARA_LEAF | p-Super | GC | 33 | 45 |
| 112072 | non-targ | B1219 | a-toco | ARA_LEAF | p-Super | GC | 51 | 98 |
| 112111 | non-targ | B1234 | g-toco | ARA_LEAF | p-Super | LC | 33 | 57 |
| 77774 | non-targ | B1479 | g-toco | ARA_LEAF | p-Super | LC | 33 | 79 |
| 7947 | non-targ | B1522 | g-toco | ARA_LEAF | p-Super | LC | 51 | 301 |
| 38300 | non-targ | B1597 | a-toco | ARA_LEAF | p-Super | GC | 50 | 302 |
| 7992 | non-targ | B1601 | a-toco | ARA_LEAF | p-Super | GC | 52 | 112 |
| 79056 | non-targ | B1897 | g-toco | ARA_LEAF | p-Super | LC | 36 | 153 |
| 112169 | non-targ | B1921 | g-toco | ARA_LEAF | p-Super | LC | 46 | 84 |
| 79217 | non-targ | B2027 | g-toco | ARA_LEAF | p-Super | LC | 39 | 203 |
| 112193 | non-targ | B2043 | a-toco | ARA_LEAF | p-Super | LC | 38 | 60 |
| 39219 | non-targ | B2360 | g-toco | ARA_LEAF | p-Super | LC | 48 | 93 |
| 99898 | non-targ | B2546 | a-toco | ARA_LEAF | p-Super | GC | 53 | 87 |
| 40637 | non-targ | B2548 | g-toco | ARA_LEAF | p-Super | LC | 35 | 100 |
| 40665 | non-targ | B2613 | g-toco | ARA_LEAF | p-Super | LC | 39 | 100 |
| 9244 | non-targ | B2673 | g-toco | ARA_LEAF | p-Super | LC | 36 | 55 |
| 80756 | non-targ | B2701 | g-toco | ARA_LEAF | p-Super | LC | 49 | 174 |
| 40795 | non-targ | B2812 | g-toco | ARA_LEAF | p-Super | LC | 34 | 83 |
| 81576 | non-targ | B2818 | a-toco | ARA_LEAF | p-Super | LC | 36 | 39 |
| 81576 | non-targ | B2818 | g-toco | ARA_LEAF | p-Super | LC | 33 | 153 |
| 112216 | non-targ | B2977 | a-toco | ARA_LEAF | p-Super | LC | 42 | 53 |
| 112380 | plastidic | B3194 | a-toco | ARA_LEAF | p-Super | LC | 25 | 26 |
| 112606 | non-targ | B3790 | g-toco | ARA_LEAF | p-Super | LC | 35 | 61 |
| 112634 | non-targ | B3890 | a-toco | ARA_LEAF | p-Super | GC | 67 | 290 |
| 44223 | non-targ | B3945 | a-toco | ARA_SEED_2 | p-USP | LC | 31 | 56 |
| 44372 | non-targ | B3989 | a-toco | ARA_LEAF | p-Super | GC | 55 | 120 |
| 44372 | non-targ | B3989 | g-toco | ARA_LEAF | p-Super | LC | 35 | 102 |
| 10740 | non-targ | B4029 | g-toco | ARA_LEAF | p-Super | LC | 38 | 244 |
| 106532 | non-targ | B4050 | a-toco | ARA_LEAF | p-Super | LC | 27 | 39 |
| 94542 | non-targ | B4056 | a-toco | ARA_LEAF | p-Super | LC | 41 | 55 |
| 45321 | non-targ | B4256 | a-toco | ARA_LEAF | p-Super | LC | 69 | 93 |
| 45321 | non-targ | B4256 | g-toco | ARA_LEAF | p-Super | LC | 61 | 132 |
| 45394 | non-targ | B4321 | a-toco | ARA_LEAF | p-Super | LC | 39 | 72 |
| 45394 | non-targ | B4321 | g-toco | ARA_LEAF | p-Super | LC | 97 | 248 |
| 45757 | non-targ | C_pp004096192r | g-toco | ARA_LEAF | p-Super | LC | 35 | 147 |
| 10811 | non-targ | GM02LC12622 | a-toco | ARA_LEAF | p-PcUBI | GC | 57 | 105 |
| 10811 | non-targ | GM02LC12622 | g-toco | ARA_LEAF | p-PcUBI | LC | 34 | 204 |
| 84198 | non-targ | GM02LC21368 | g-toco | ARA_LEAF | p-PcUBI | LC | 48 | 185 |
| 49828 | non-targ | Sll0290 | a-toco | ARA_LEAF | p-PcUBI | LC | 27 | 49 |
| 112661 | mitochondrial | Sll0745 | g-toco | ARA_LEAF | p-PcUBI | LC | 33 | 76 |
| 53189 | non-targ | Sll1185 | g-toco | ARA_LEAF | p-PcUBI | LC | 28 | 42 |
| 55379 | non-targ | Sll1761 | g-toco | ARA_LEAF | p-PcUBI | LC | 33 | 68 |
| 85808 | mitochondrial | Sll1815 | g-toco | ARA_LEAF | p-PcUBI | LC | 32 | 45 |
| 86540 | mitochondrial | Slr0239 | a-toco | ARA_LEAF | p-PcUBI | LC | 39 | 46 |
| 87397 | plastidic | Slr0721 | a-toco | ARA_LEAF | p-PcUBI | GC | 60 | 125 |
| 87397 | plastidic | Slr0721 | g-toco | ARA_LEAF | p-PcUBI | LC | 43 | 268 |
| 57235 | plastidic | Slr0739 | a-toco | ARA_LEAF | p-PcUBI | LC | 45 | 82 |
| 57235 | plastidic | Slr0739 | g-toco | ARA_LEAF | p-PcUBI | LC | 91 | 144 |
| 58590 | non-targ | Slr1420 | g-toco | ARA_LEAF | p-PcUBI | LC | 33 | 50 |
| 58731 | plastidic | Slr1739 | g-toco | ARA_LEAF | p-PcUBI | LC | 77 | 203 |
| 58823 | plastidic | Slr1755 | a-toco | ARA_LEAF | p-PcUBI | LC | 37 | 81 |
| 12140 | plastidic | Slr1791 | g-toco | ARA_LEAF | p-PcUBI | LC | 33 | 58 |
| 113038 | plastidic | Slr1917 | a-toco | ARA_LEAF | p-PcUBI | LC | 36 | 70 |
| 113038 | plastidic | Slr1917 | g-toco | ARA_LEAF | p-PcUBI | LC | 144 | 540 |
| 12698 | non-targ | TTC0019 | a-toco | ARA_LEAF | p-PcUBI | GC | 53 | 129 |
| 12698 | non-targ | TTC0019 | g-toco | ARA_LEAF | p-PcUBI | LC | 35 | 264 |
| 61723 | non-targ | TTC1193 | g-toco | ARA_LEAF | p-PcUBI | LC | 88 | 166 |
| 62079 | non-targ | TTC1386 | a-toco | ARA_LEAF | p-PcUBI | GC | 52 | 132 |
| 62079 | non-targ | TTC1386 | g-toco | ARA_LEAF | p-PcUBI | LC | 38 | 171 |
| 12974 | non-targ | TTC1550 | a-toco | ARA_LEAF | p-PcUBI | GC | 86 | 106 |
| 88879 | plastidic | Ybr249c | a-toco | ARA_LEAF | p-Super | GC | 76 | 142 |
| 88879 | plastidic | Ybr249c | g-toco | ARA_LEAF | p-Super | LC | 51 | 168 |
| 113220 | plastidic | Ycl049c | g-toco | ARA_LEAF | p-Super | LC | 57 | 92 |
| 113224 | plastidic | Ydl131w | a-toco | ARA_LEAF | p-Super | GC | 66 | 285 |
| 113313 | plastidic | Ydl235c | a-toco | ARA_LEAF | p-Super | GC | 55 | 199 |
| 89973 | plastidic | Ydr007w | g-toco | ARA_LEAF | p-Super | LC | 70 | 140 |
| 113330 | plastidic | Ydr035w | g-toco | ARA_LEAF | p-Super | LC | 39 | 106 |
| 90103 | plastidic | Ydr183w | g-toco | ARA_LEAF | p-Super | LC | 35 | 105 |
| 63803 | plastidic | Yer106w | g-toco | ARA_LEAF | p-Super | LC | 34 | 302 |
| 113598 | non-targ | Yer112w | g-toco | ARA_LEAF | Big35S | GC | 51 | 252 |
| 113636 | plastidic | Ygr170w | a-toco | ARA_LEAF | p-PcUBI | LC | 42 | 62 |
| 66274 | non-targ | Yjr153w | g-toco | ARA_LEAF | p-PcUBI | LC | 89 | 115 |
| 66715 | plastidic | Ylr178c | g-toco | ARA_LEAF | p-Super | LC | 39 | 62 |
| 67190 | plastidic | Ylr359w | a-toco | ARA_LEAF | p-Super | GC | 54 | 78 |
| 113647 | plastidic | Ypr170c | a-toco | ARA_LEAF | p-Super | GC | 59 | 73 |

Column 1 shows the SEQ ID NO, Column 2 shows the expression type (targeted or non-targeted), Column 3 shows the “gene name” (locus), Column 4 shows the metabolite analyzed, Column 5 indicates the A. thaliana source tissue analyzed, Column 6 indicates the used promoter for expression, Column 7 indicates the analytical method. Columns 8 and 9 show the minimum and the maximum increase of the analyzed metabolite (in-percent) in comparison to the wild type (ratio_by_WT, given as-percent increase).

The term “non-targ” in Table d, Column 2 which shows the expression type means “non-targeted”. The term “a-toco” in Table d, Column 4 which shows the metabolite means “alpha-tocopherol”.

The term “g-toco” in Table d, Column 4 which shows the metabolite means “gamma-tocopherol”.

When the analyses were repeated independently, all results proved to be significant.

### Example 12

### Engineering Arabidopsis Plants with an Increased Production of a Fine Chemical by (Over)Expressing a FCRP-Protein Encoded by a Gene from Saccharomyces Cerevisiae or Synechocystis or E. Coli, Azotobacter Vinelandii, Thermus Thermophilus, Physcomitrella Patens, Arabidopsis. Thaliana, Brassica Napus, Glycine Max, Zea Mays or Oryza Sativa using Tissue-Specific and/or Stress Inducible Promoters

Transgenic Arabidopsis plants are created as in example 11 to express the FCRP under the control of a tissue-specific and/or stress inducible promoter.

T2 generation plants are produced and are grown under standard conditions. The fine chemical production is determined after a total time of 29 to 30 days starting with the sowing. The transgenic Arabidopsis plant produces more fine chemical then non-transgenic control plants.

### Example 13

### Engineering Alfalfa Plants with an Increased Production of a Fine Chemical by (Over)Expressing a FCRP-Protein Encoded by a Gene from Saccharomyces Cerevisiae or Synechocystis or E. Coli, Azotobacter Vinelandii, Thermus Thermophilus, Physcomitrella Patens, Arabidopsis. Thaliana, Brassica Napus, Glycine Max, Zea Mays or Oryza Sativa

A regenerating clone of alfalfa (Medicago sativa) is transformed using the method of (McKersie et al., Plant Physiol 119, 839 (1999)). Regeneration and transformation of alfalfa is genotype dependent and therefore a regenerating plant is required. Methods to obtain regenerating plants have been described. For example, these can be selected from the cultivar Rangelander (Agriculture Canada) or any other commercial alfalfa variety as described by Brown D. C. W. and Atanassov A. (Plant Cell Tissue Organ Culture 4, 111 (1985)). Alternatively, the RA3 variety (University of Wisconsin) is selected for use in tissue culture (Walker et al., Am. J. Bot. 65, 654 (1978)).

Petiole explants are cocultivated with an overnight culture of Agrobacterium tumefaciens C58C1 pMP90 (McKersie et al., Plant Physiol 119, 839 (1999)) or LBA4404 containing a binary vector. Many different binary vector systems have been described for plant transformation (e.g. G. An, in “Agrobacterium Protocols, Methods in Molecular Biology”, Vol. 44, pp. 47-62, Gartland K. M. A. and Davey M. R., eds. Humana Press, Totowa, N.J.). Many are based on the vector pBIN19 described by Bevan (Nucleic Acid Research. 12, 8711 (1984)) that includes a plant gene expression cassette flanked by the left and right border sequences from the Ti plasmid of Agrobacterium tumefaciens. A plant gene expression cassette consists of at least two genes—a selection marker gene and a plant promoter regulating the transcription of the cDNA or genomic DNA of the trait gene. Various selection marker genes can be used including the Arabidopsis gene encoding a mutated acetohydroxy acid synthase (AHAS) enzyme (U.S. Pat. No. 5,7673,666 and U.S. Pat. No. 6,225,105). Similarly, various promoters can be used to regulate the trait gene that provides constitutive, developmental, tissue or environmental regulation of gene transcription. In this example, the 34S promoter (GenBank Accession numbers M59930 and X16673) is used to provide constitutive expression of the trait gene.

The explants are cocultivated for 3 days in the dark on SH induction medium containing 288 mg/ L Pro, 53 mg/ L thioproline, 4.35 g/L K_{2SO}₄, and 100 μm acetosyringinone. The explants are washed in half-strength Murashige-Skoog medium (Murashige and Skoog, 1962) and plated on the same SH induction medium without acetosyringinone but with a suitable selection agent and suitable antibiotic to inhibit Agrobacterium growth. After several weeks, somatic embryos are transferred to BOi2Y development medium containing no growth regulators, no antibiotics, and 50 g/L sucrose. Somatic embryos are subsequently germinated on half-strength Murashige-Skoog medium. Rooted seedlings are transplanted into pots and grown in a greenhouse.

T1 or T2 generation plants are produced, grown and processed and evaluated for their fine chemical produced in analogy as described above.

### Example 14

### Engineering Ryegrass Plants with an Increased Production of Fine Chemical by (Over)-Expressing a FCRP Encoded by a Gene from Saccharomyces Cerevisiae or Synechocystis or E. Coli, Azotobacter Vinelandii, Thermus Thermophilus, Physcomitrella Patens, Arabidopsis. Thaliana, Brassica Napus, Glycine Max, Zea Mays or Oryza Sativa

Seeds of several different ryegrass varieties may be used as explant sources for transformation, including the commercial variety Gunne available from Svalof Weibull seed company or the variety Affinity. Seeds are surface-sterilized sequentially with 1% Tween-20 for 1 minute, 100% bleach for 60 minutes, 3 rinses with 5 minutes each with deionized and distilled H2O, and then germinated for 3-4 days on moist, sterile filter paper in the dark. Seedlings are further sterilized for 1 minute with 1% Tween-20, 5 minutes with 75% bleach, and rinsed 3 times with double distilled H₂O, 5 min each.

Surface-sterilized seeds are placed on the callus induction medium containing Murashige and Skoog basal salts and vitamins, 20 g/L sucrose, 150 mg/L asparagine, 500 mg/L casein hydrolysate, 3 g/L Phytagel, 10 mg/L BAP, and 5 mg/L dicamba. Plates are incubated in the dark at 25° C. for 4 weeks for seed germination and embryogenic callus induction.

After 4 weeks on the callus induction medium, the shoots and roots of the seedlings are trimmed away, the callus is transferred to fresh media, maintained in culture for another 4 weeks, and then transferred to MSO medium in light for 2 weeks. Several pieces of callus (11-17 weeks old) are either strained through a 10 mesh sieve and put onto callus induction medium, or cultured in 100 ml of liquid ryegrass callus induction media (same medium as for callus induction with agar) in a 250 ml flask. The flask is wrapped in foil and shaken at 175 rpm in the dark at 23° C. for 1 week. Sieving the liquid culture with a 40-mesh sieve collected the cells. The fraction collected on the sieve is plated and cultured on solid ryegrass callus induction medium for 1 week in the dark at 25° C. The callus is then transferred to and cultured on MS medium containing 1% sucrose for 2 weeks.

Transformation can be accomplished with either Agrobacterium of with particle bombardment methods. An expression vector is created containing a constitutive plant promoter and the cDNA of the gene in a pUC vector. The plasmid DNA is prepared from E. coli cells using with

Qiagen kit according to manufacturer's instruction. Approximately 2 g of embryogenic callus is spread in the center of a sterile filter paper in a Petri dish. An aliquot of liquid MSO with 10 g/L sucrose is added to the filter paper. Gold particles (1.0 pm in size) are coated with plasmid DNA according to method of Sanford et al., 1993 and delivered to the embryogenic callus with the following parameters: 500 pg particles and 2 pg DNA per shot, 1300 psi and a target distance of 8.5 cm from stopping plate to plate of callus and 1 shot per plate of callus.

After the bombardment, calli are transferred back to the fresh callus development medium and maintained in the dark at room temperature for a 1-week period. The callus is then transferred to growth conditions in the light at 25° C. to initiate embryo differentiation with the appropriate selection agent, e.g. 250 nM Arsenal, 5 mg/L PPT or 50 mg/L kanamycin. Shoots resistant to the selection agent are appearing and once rooted are transferred to soil.

Samples of the primary transgenic plants (T0) are analyzed by PCR to confirm the presence of T-DNA. These results are confirmed by Southern hybridization in which DNA is electrophoresed on a 1% agarose gel and transferred to a positively charged nylon membrane (Roche Diagnostics). The PCR DIG Probe Synthesis Kit (Roche Diagnostics) is used to prepare a digoxigeninlabelled probe by PCR, and used as recommended by the manufacturer.

Transgenic T0 ryegrass plants are propagated vegetatively by excising tillers. The transplanted tillers are maintained in the greenhouse for 2 months until well established. The shoots are defoliated and allowed to grow for 2 weeks.

T1 or T2 generation plants are produced, grown and processed and evaluated for their fine chemical produced in analogy as described above to determine the fine chemical content.

### Example 15

### Engineering Soybean Plants with an Increased Production of Fine Chemical by (Over)-Expressing a FCRP Encoded by a Gene from Saccharomyces Cerevisiae or Synechocystis or E. Coli, Azotobacter Vinelandii, Thermus Thermophilus, Physcomitrella Patens, Arabidopsis. Thaliana, Brassica Napus, Glycine Max, Zea Mays or Oryza Sativa

Soybean is transformed according to the following modification of the method described in the Texas A & M U.S. Pat. No. 5,164,310. Several commercial soybean varieties are amenable to transformation by this method. The cultivar Jack (available from the Illinois Seed Foundation) is commonly used for transformation. Seeds are sterilized by immersion in 70% (v/v) ethanol for 6 min and in 25% commercial bleach (NaOCI) supplemented with 0.1% (v/v) Tween for 20 min, followed by rinsing 4 times with sterile double distilled water. Seven-day seedlings are propagated by removing the radicle, hypocotyl and one cotyledon from each seedling. Then, the epicotyl with one cotyledon is transferred to fresh germination media in petri dishes and incubated at 25° C. under a 16-h photoperiod (approx. 100 μmol m⁻²s−1) for three weeks. Axillary nodes (approx. 4 mm in length) were cut from 3-4 week-old plants. Axillary nodes are excised and incubated in Agrobacterium LBA4404 culture.

Many different binary vector systems have been described for plant transformation (e.g. An G., in Agrobacterium Protocols. Methods in Molecular Biology Vol. 44, pp. 47-62, Gartland K.M.A. and Davey M. R. eds. Humana Press, Totowa, N.Jersey.). Many are based on the vector pBIN19 described by Bevan (Nucleic Acid Research. 12, 8711 (1984)) that includes a plant gene expression cassette flanked by the left and right border sequences from the Ti plasmid of Agrobacterium tumefaciens. A plant gene expression cassette consists of at least two genes—a selection marker gene and a plant promoter regulating the transcription of the cDNA or genomic DNA of the trait gene. Various selection marker genes can be used including the Arabidopsis gene encoding a mutated acetohydroxy acid synthase (AHAS) enzyme (U.S. Pat. No. 5,7673,666 and U.S. Pat. No. 6,225,105). Similarly, various promoters can be used to regulate the trait gene to provide constitutive, developmental, tissue or environmental regulation of gene transcription. In this example, the 34S promoter (GenBank Accession numbers M59930 and X16673) can be used to provide constitutive expression of the trait gene.

After the co-cultivation treatment, the explants are washed and transferred to selection media supplemented with 500 mg/L timentin. Shoots are excised and placed on a shoot elongation medium. Shoots longer than 1 cm are placed on rooting medium for two to four weeks prior to transplanting to soil.

The primary transgenic plants (TO) are analyzed by PCR to confirm the presence of T-DNA.

These results are confirmed by Southern hybridization in which DNA is electrophoresed on a 1% agarose gel and transferred to a positively charged nylon membrane (Roche Diagnostics). The PCR DIG Probe Synthesis Kit (Roche Diagnostics) is used to prepare a digoxigeninlabelled probe by PCR, and used as recommended by the manufacturer.

T1 or T2 generation plants are produced, grown and processed and evaluated for their fine chemical produced in analogy as described above.

### Example 16

### Engineering Rapeseed/Canola Plants with an Increased Production of Fine Chemical by (Over)-Expressing a FCRP Encoded by a Gene from Saccharomyces Cerevisiae or Synechocystis or E. Coli, Azotobacter Vinelandii, Thermus Thermophilus, Physcomitrella Patens, Arabidopsis. Thaliana, Brassica Napus, Glycine Max, Zea Mays or Oryza Sativa

Cotyledonary petioles and hypocotyls of 5-6 day-old young seedlings are used as explants for tissue culture and transformed according to Babic et al. (Plant Cell Rep 17, 183 (1998)). The commercial cultivar Westar (Agriculture Canada) is the standard variety used for transformation, but other varieties can be used.

Agrobacterium tumefaciens LBA4404 containing a binary vector can be used for canola transformation. Many different binary vector systems have been described for plant transformation (e.g. An G., in Agrobacterium Protocols. Methods in Molecular Biology Vol. 44, p. 47-62, Gartland K. M. A. and Davey M. R. eds. Humana Press, Totowa, N.J.). Many are based on the vector pBIN19 described by Bevan (Nucleic Acid Research. 12, 8711(1984)) that includes a plant gene expression cassette flanked by the left and right border sequences from the Ti plasmid of Agrobacterium tumefaciens. A plant gene expression cassette consists of at least two genes—a selection marker gene and a plant promoter regulating the transcription of the cDNA or genomic DNA of the trait gene. Various selection marker genes can be used including the Arabidopsis gene encoding a mutated acetohydroxy acid synthase (AHAS) enzyme (U.S. Pat. No. 5,7673,666 and U.S. Pat. No. 6,225,105). Similarly, various promoters can be used to regulate the trait gene to provide constitutive, developmental, tissue or environmental regulation of gene transcription. In this example, the 34S promoter (GenBank Accession numbers M59930 and X16673) can be used to provide constitutive expression of the trait gene.

Canola seeds are surface-sterilized in 70% ethanol for 2 min., and then in 30% Clorox with a drop of Tween-20 for 10 min, followed by three rinses with sterilized distilled water. Seeds are then germinated in vitro 5 days on half strength MS medium without hormones, 1% sucrose, 0.7% Phytagar at 23° C., 16 h light. The cotyledon petiole explants with the cotyledon attached are excised from the in vitro seedlings, and inoculated with Agrobacterium by dipping the cut end of the petiole explant into the bacterial suspension. The explants are then cultured for 2 days on MSBAP-3 medium containing 3 mg/L BAP, 3% sucrose, 0.7% Phytagar at 23° C., 16 h light. After two days of co-cultivation with Agrobacterium, the petiole explants are transferred to MSBAP-3 medium containing 3 mg/L BAP, cefotaxime, carbenicillin, or timentin (300 mg/L) for 7 days, and then cultured on MSBAP-3 medium with cefotaxime, carbenicillin, or timentin and selection agent until shoot regeneration. When the shoots were 5-10 mm in length, they are cut and transferred to shoot elongation medium (MSBAP-0.5, containing 0.5 mg/L BAP). Shoots of about 2 cm in length are transferred to the rooting medium (MSO) for root induction.

Samples of the primary transgenic plants (TO) are analyzed by PCR to confirm the presence of T-DNA. These results are confirmed by Southern hybridization in which DNA is electrophoresed on a 1% agarose gel and transferred to a positively charged nylon membrane (Roche Diagnostics). The PCR DIG Probe Synthesis Kit (Roche Diagnostics) is used to prepare a digoxigeninlabelled probe by PCR, and used as recommended by the manufacturer.

T1 or T2 generation plants are produced, grown and processed and evaluated for their fine chemical produced in analogy as described above.

### Example 17

### Engineering Corn Plants with an Increased Production of Fine Chemical by (Over)-Expressing a FCRP Encoded by a Gene from Saccharomyces Cerevisiae or Synechocystis or E. Coli, Azotobacter Vinelandii, Thermus Thermophilus, Physcomitrella Patens, Arabidopsis. Thaliana, Brassica Napus, Glycine Max, Zea Mays or Oryza Sativa

Transformation of maize (Zea Mays L.) is performed with a modification of the method described by Ishida et al. (Nature Biotech 14745 (1996)). Transformation is genotype-dependent in corn and only specific genotypes are amenable to transformation and regeneration. The inbred line A188 (University of Minnesota) or hybrids with A188 as a parent are good sources of donor material for transformation (Fromm et al., Biotech 8, 833 (1990)), but other genotypes can be used successfully as well. Ears are harvested from corn plants at approximately 11 days after pollination (DAP) when the length of immature embryos is about 1 to 1.2 mm. Immature embryos are co-cultivated with Agrobacterium tumefaciens that carry “super binary” vectors and transgenic plants are recovered through organogenesis. The super binary vector system of Japan Tobacco is described in WO patents WO 94/00977 and WO 95/06722. Vectors were constructed as described. Various selection marker genes can be used including the maize gene encoding a mutated acetohydroxy acid synthase (AHAS) enzyme (U.S. Pat. No. 6,025,541). Similarly, various promoters can be used to regulate the trait gene to provide constitutive, developmental, tissue or environmental regulation of gene transcription. In this example, the 34S promoter (GenBank Accession numbers M59930 and X16673) was used to provide constitutive expression of the trait gene.

Excised embryos are grown on callus induction medium, then maize regeneration medium, containing imidazolinone as a selection agent. The Petri plates are incubated in the light at 25° C. for 2-3 weeks, or until shoots develop. The green shoots are transferred from each embryo to maize rooting medium and incubated at 25° C. for 2-3 weeks, until roots develop. The rooted shoots are transplanted to soil in the greenhouse. T1 seeds are produced from plants that exhibit tolerance to the imidazolinone herbicides and which are PCR positive for the transgenes.

The T1 transgenic plants are then evaluated for their fine chemical producted as described above. The T1 generation of single locus insertions of the T-DNA will segregate for the transgene in a 3:1 ratio. Those progeny containing one or two copies of the transgene are tolerant regarding the imidazolinone herbicide, and exhibit an increased fine chemical production then those progeny lacking the transgenes.

T1 or T2 generation plants are produced, grown, processed and evaluated for their fine chemical produced in analogy as described above. Homozygous T2 plants exhibited similar phenotypes. Hybrid plants (F1 progeny) of homozygous transgenic plants and non-transgenic plants also exhibited enhanced fine chemical production.

### Example 18

### Engineering Wheat Plants with an Increased Production of Fine Chemical by (Over)-Expressing a FCRP Encoded by a Gene from Saccharomyces cerevisiae or Synechocystis or E. Coli, Azotobacter Vinelandii, Thermus Thermophilus, Physcomitrella Patens, Arabidopsis. Thaliana, Brassica Napus, Glycine Max, Zea Mays or Oryza Sativa

Transformation of wheat is performed with the method described by Ishida et al. (Nature Biotech. 14745 (1996)). The cultivar Bobwhite (available from CYMMIT, Mexico) is commonly used in transformation. Immature embryos are co-cultivated with Agrobacterium tumefaciens that carry “super binary” vectors, and transgenic plants are recovered through organogenesis. The super binary vector system of Japan Tobacco is described in WO patents WO 94/00977 and WO 95/06722. Vectors were constructed as described. Various selection marker genes can be used including the maize gene encoding a mutated acetohydroxy acid synthase (AHAS) enzyme (U.S. Pat. No. 6,025,541). Similarly, various promoters can be used to regulate the trait gene to provide constitutive, developmental, tissue or environmental regulation of gene transcription. In this example, the 34S promoter (GenBank Accession numbers M59930 and X16673) was used to provide constitutive expression of the trait gene.

After incubation with Agrobacterium, the embryos are grown on callus induction medium, then regeneration medium, containing imidazolinone as a selection agent. The Petri plates are incubated in the light at 25° C. for 2-3 weeks, or until shoots develop. The green shoots are transferred from each embryo to rooting medium and incubated at 25° C. for 2-3 weeks, until roots develop. The rooted shoots are transplanted to soil in the greenhouse. T1 seeds are produced from plants that exhibit tolerance to the imidazolinone herbicides and which are PCR positive for the transgenes.

The T1 transgenic plants are then evaluated for their enhanced production of fine chemical according to the methods described in the previous examples. The T1 generation of single locus insertions of the T-DNA will segregate for the transgene in a 3:1 ratio. Those progeny containing one or two copies of the transgene are tolerant regarding the imidazolinone herbicide, and exhibit an enhanced production of fine chemical then those progeny lacking the transgenes. Homozygous T2 plants exhibited similar phenotypes.

### Example 19

### Engineering Rice Plants with an Increased Production of Fine Chemical by (Over)-Expressing a FCRP Encoded by a Gene from Saccharomyces Cerevisiae or Synechocystis or E. Coli, Azotobacter Vinelandii, Thermus Thermophilus, Physcomitrella Patens, Arabidopsis. Thaliana, Brassica Napus, Glycine Max, Zea Mays or Oryza Sativa

The Agrobacterium containing the expression vector of the invention is used to transform Oryza sativa plants. Mature dry seeds of the rice japonica cultivar Nipponbare are dehusked. Sterilization is carried out by incubating for one minute in 70% ethanol, followed by 30 minutes in 0.2% HgCl₂, followed by a 6 times 15 minutes can beh with sterile distilled water. The sterile seeds are then germinated on a medium containing 2,4-D (callus induction medium). After incubation in the dark for four weeks, embryogenic, scutellum-derived calli are excised and propagated on the same medium. After two weeks, the calli are multiplied or propagated by subculture on the same medium for another 2 weeks. Embryogenic callus pieces are sub-cultured on fresh medium 3 days before co-cultivation (to boost cell division activity).

Agrobacterium strain LBA4404 containing the expression vector of the invention is used for cocultivation. Agrobacterium is inoculated on AB medium with the appropriate antibiotics and cultured for 3 days at 28° C. The bacteria are then collected and suspended in liquid co-cultivation medium to a density (0D600) of about 1. The suspension is then transferred to a Petri dish and the calli immersed in the suspension for 15 minutes. The callus tissues are then blotted dry on a filter paper and transferred to solidified, co-cultivation medium and incubated for 3 days in the dark at 25° C. Co-cultivated calli are grown on 2,4-D-containing medium for 4 weeks in the dark at 28° C. in the presence of a selection agent. During this period, rapidly growing resistant callus islands developed. After transfer of this material to a regeneration medium and incubation in the light, the embryogenic potential is released and shoots developed in the next four to five weeks. Shoots are excised from the calli and incubated for 2 to 3 weeks on an auxin-containing medium from which they are transferred to soil. Hardened shoots are grown under high humidity and short days in a greenhouse.

Approximately 35 independent TO rice transformants are generated for one construct. The primary transformants are transferred from a tissue culture chamber to a greenhouse. After a quantitative PCR analysis to verify copy number of the T-DNA insert, only single copy transgenic plants that exhibit tolerance to the selection agent are kept for harvest of T1 seed. Seeds are then harvested three to five months after transplanting. The method yielded single locus transformants at a rate of over 50% (Aldemita and Hodges1996, Chan et al. 1993, Hiei et al. 1994).

T1 or T2 generation plants are produced, grown, processed and evaluated for their fine chemical produced in analogy as described above

### Example 20

### Identification of Identical and Heterologous Genes

Gene sequences can be used to identify identical or heterologous genes from cDNA or genomic libraries. Identical genes (e. g. full-length cDNA clones) can be isolated via nucleic acid hybridization using for example cDNA libraries. Depending on the abundance of the gene of interest, 100,000 up to 1,000,000 recombinant bacteriophages are plated and transferred to nylon membranes. After denaturation with alkali, DNA is immobilized on the membrane by e. g. UV cross linking. Hybridization is carried out at high stringency conditions. In aqueous solution, hybridization and washing is performed at an ionic strength of 1 M NaCl and a temperature of 68° C. Hybridization probes are generated by e.g. radioactive (32P) nick transcription labeling (High Prime, Roche, Mannheim, Germany). Signals are detected by autoradiography.

Partially identical or heterologous genes that are related but not identical can be identified in a manner analogous to the above-described procedure using low stringency hybridization and washing conditions. For aqueous hybridization, the ionic strength is normally kept at 1 M NaCl while the temperature is progressively lowered from 68 to 42° C.

Isolation of gene sequences with homology (or sequence identity/similarity) only in a distinct domain of (for example 10-20 amino acids) can be carried out by using synthetic radio labeled oligonucleotide probes. Radiolabeled oligonucleotides are prepared by phosphorylation of the 5-prime end of two complementary oligonucleotides with T4 polynucleotide kinase. The complementary oligonucleotides are annealed and ligated to form concatemers. The double stranded concatemers are than radiolabeled by, for example, nick transcription. Hybridization is normally performed at low stringency conditions using high oligonucleotide concentrations.

### Oligonucleotide Hybridization Solution:

6×SSC
0.01 M sodium phosphate
1 mM EDTA (pH 8)
0.5% SDS
100 μg/ml denatured salmon sperm DNA
0.1% nonfat dried milk

During hybridization, temperature is lowered stepwise to 5-10° C. below the estimated oligonucleotide Tm or down to room temperature followed by washing steps and autoradiography.

Washing is performed with low stringency such as 3 washing steps using 4×SSC. Further details are described by Sambrook J. et al., 1989, “Molecular Cloning: A Laboratory Manual,” Cold Spring Harbor Laboratory Press or Ausubel F.M. et al., 1994, “Current Protocols in Molecular Biology,” John Wiley & Sons.

### Example 21

### Identification of Identical Genes by Screening Expression Libraries with Antibodies

c-DNA clones can be used to produce recombinant polypeptide for example in E. coli (e.g. Qiagen QlAexpress pQE system). Recombinant polypeptides are then normally affinity purified via Ni-NTA affinity chromatography (Qiagen). Recombinant polypeptides are then used to produce specific antibodies for example by using standard techniques for rabbit immunization. Antibodies are affinity purified using a Ni-NTA column saturated with the recombinant antigen as described by Gu et al., BioTechniques 17, 257 (1994). The antibody can than be used to screen expression cDNA libraries to identify identical or heterologous genes via an immunological screening (Sambrook J., et al., “Molecular Cloning: A Laboratory Manual,” Cold Spring Harbor Laboratory Press, 1989, or Ausubel F. M. et al., “Current Protocols in Molecular Biology”, John Wiley & Sons, 1994,).

### FIGURES

FIG. 1. Vector VC-MME220-1 qcz (SEQ ID NO: 41) used for cloning gene of interest for non-targeted expression.

FIG. 2. Vector VC-MME221-1qcz (SEQ ID NO: 46) used for cloning gene of interest for non-targeted expression.

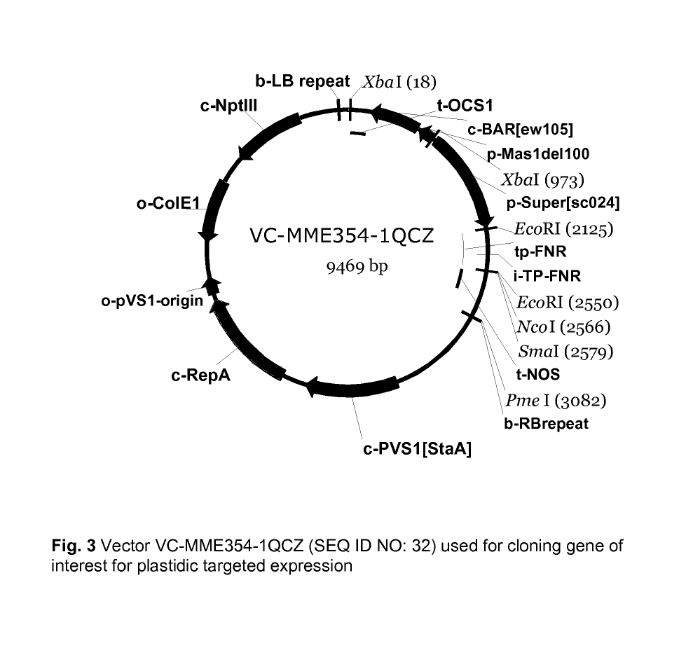

FIG. 3. Vector VC-MME354-1 QCZ (SEQ ID NO: 32) used for cloning gene of interest for plastidic targeted expression.

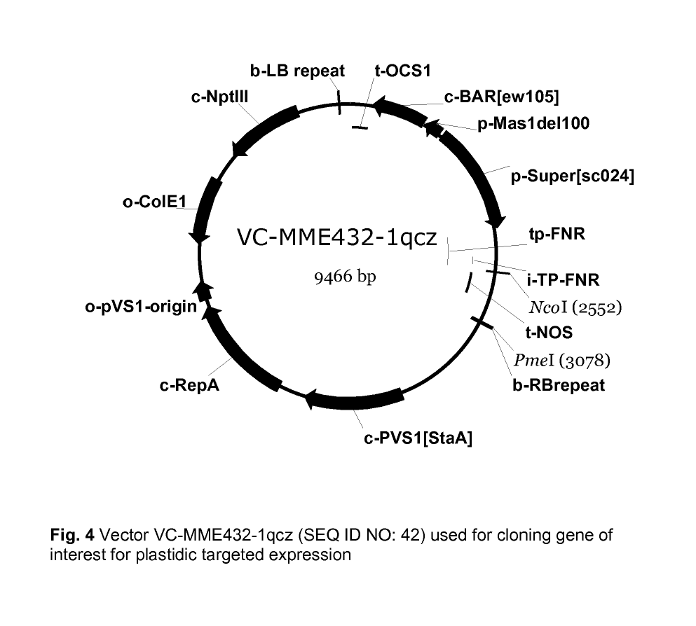

FIG. 4. Vector VC-MME432-1 qcz (SEQ ID NO: 42) used for cloning gene of interest for plastidic targeted expression.

FIG. 5. Vector VC-MME489-1 QCZ (SEQ ID NO: 56) used for cloning gene of interest for non-targeted expression and cloning of a targeting sequence.

FIG. 6. Vector pMTX0270p (SEQ ID NO: 30) used for cloning of a targeting sequence.

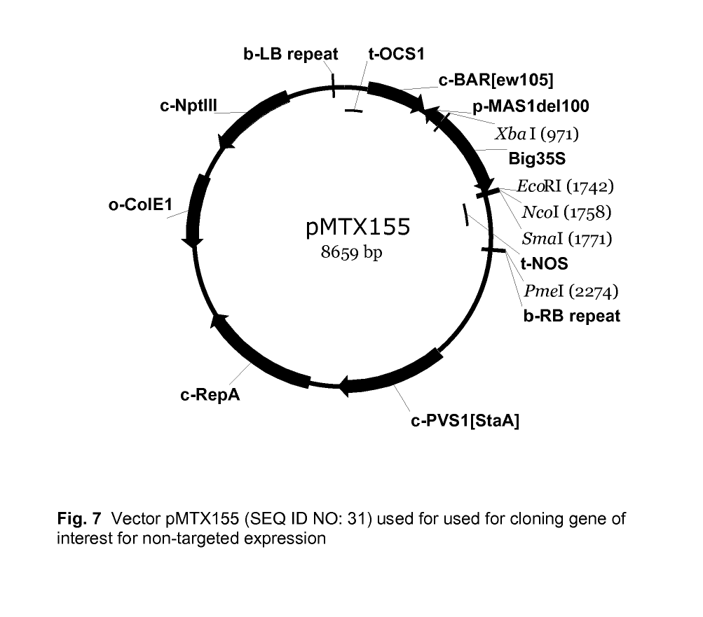

FIG. 7. Vector pMTX155 (SEQ ID NO: 31) used for used for cloning gene of interest for non-targeted expression.

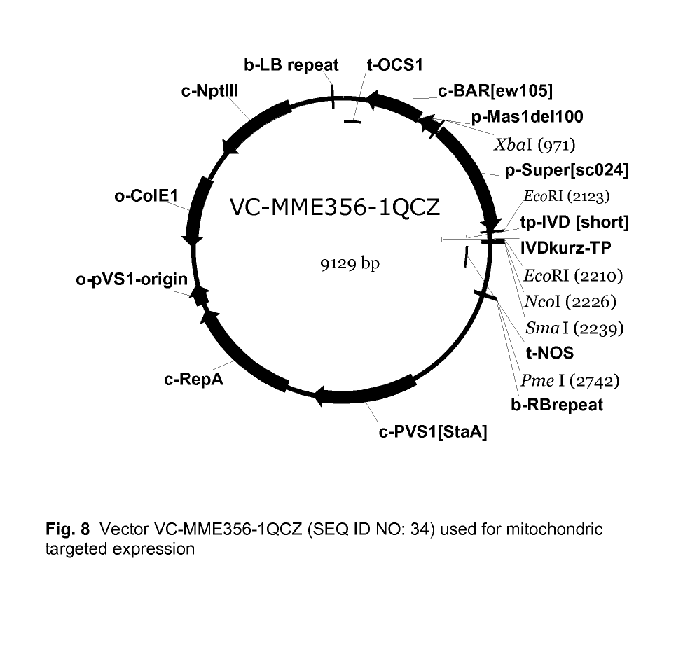

FIG. 8. Vector VC-MME356-1 QCZ (SEQ ID NO: 34) used for mitochondric targeted expression.

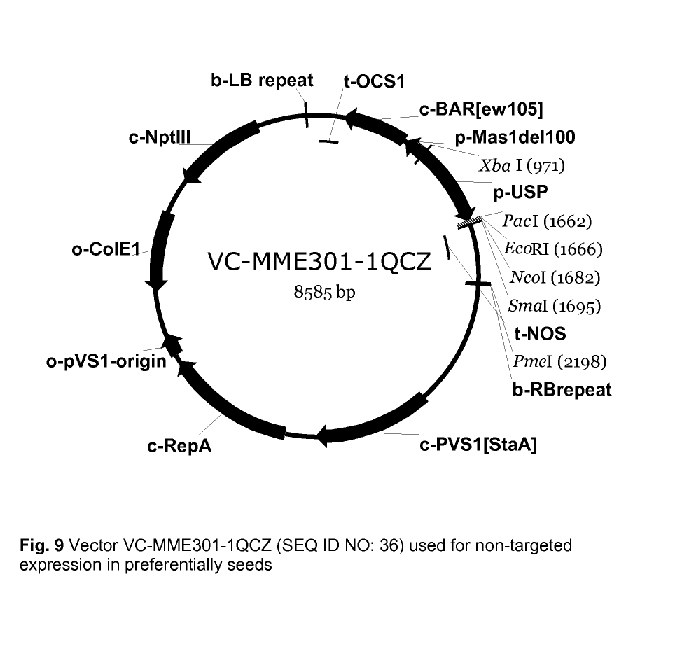

FIG. 9. Vector VC-MME301-1 QCZ (SEQ ID NO: 36) used for non-targeted expression in preferentially seeds.

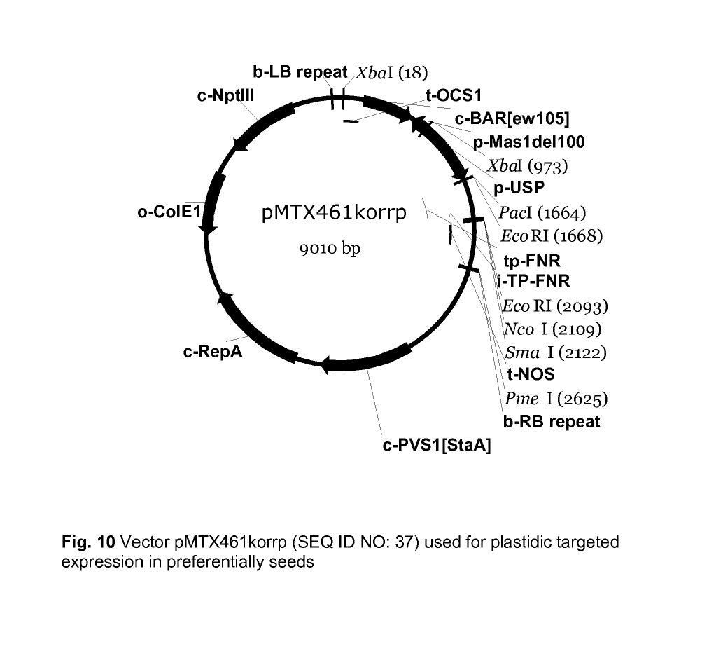

FIG. 10. Vector pMTX461korrp (SEQ ID NO: 37) used for plastidic targeted expression in preferentially seeds.

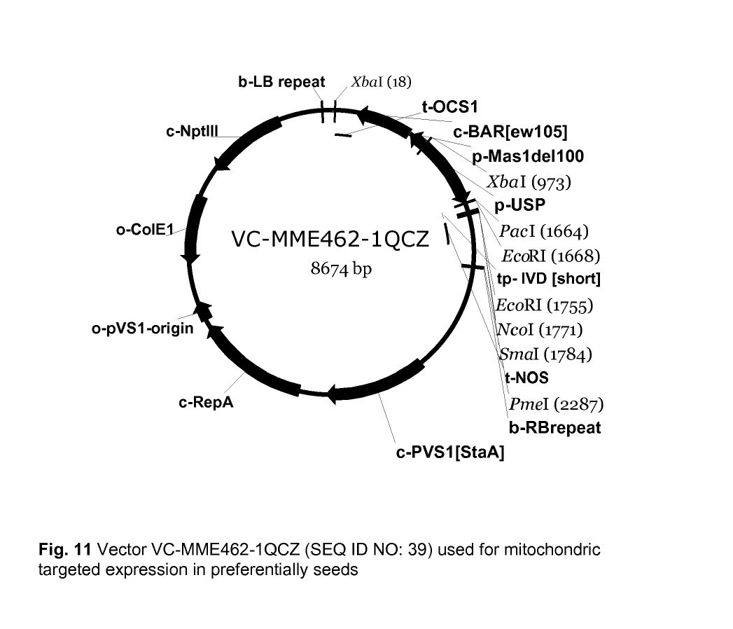

FIG. 11. Vector VC-MME462-1 QCZ (SEQ ID NO: 39) used for mitochondric targeted expression in preferentially seeds.

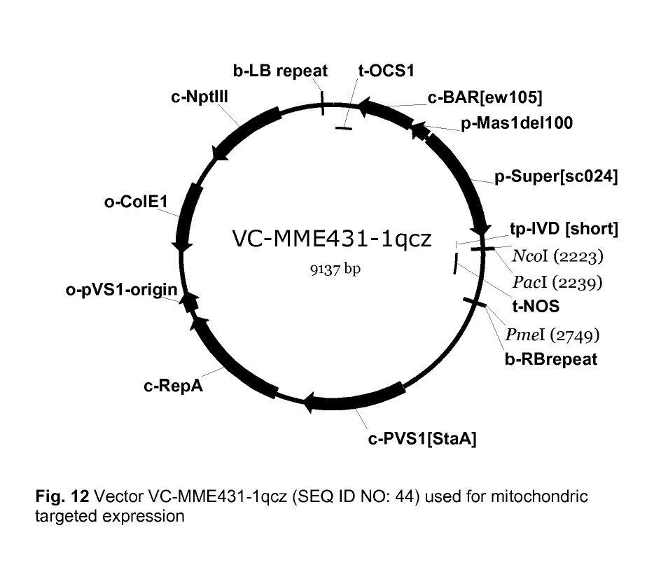

FIG. 12. Vector VC-MME431-1qcz (SEQ ID NO: 44) used for mitochondric targeted expression.

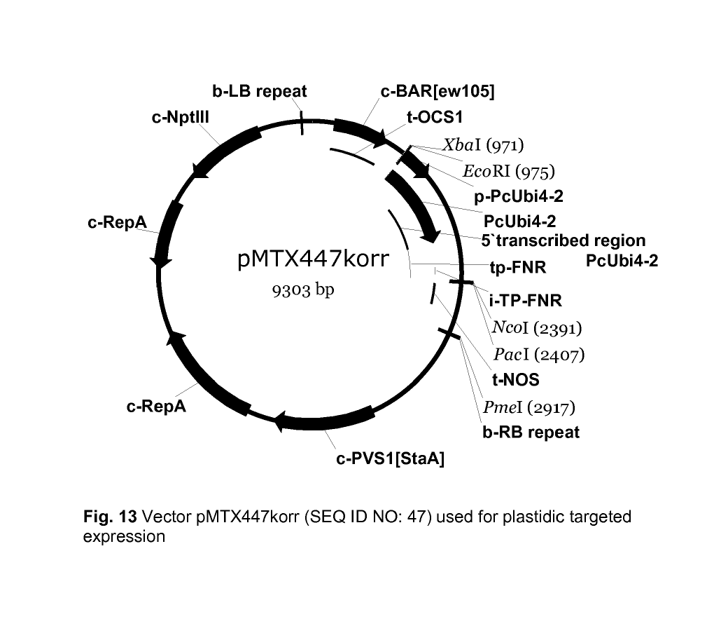

FIG. 13. Vector pMTX447korr (SEQ ID NO: 47) used for plastidic targeted expression.

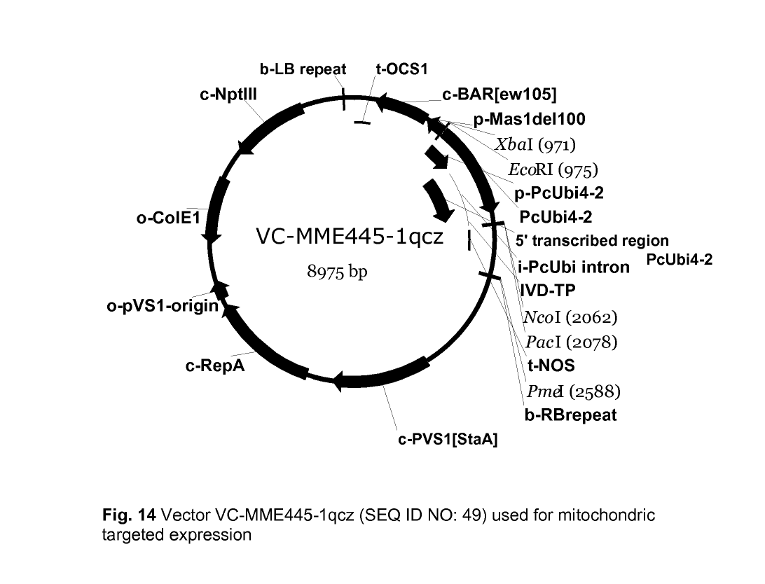

FIG. 14. Vector VC-MME445-1 qcz (SEQ ID NO: 49) used for mitochondric targeted expression.

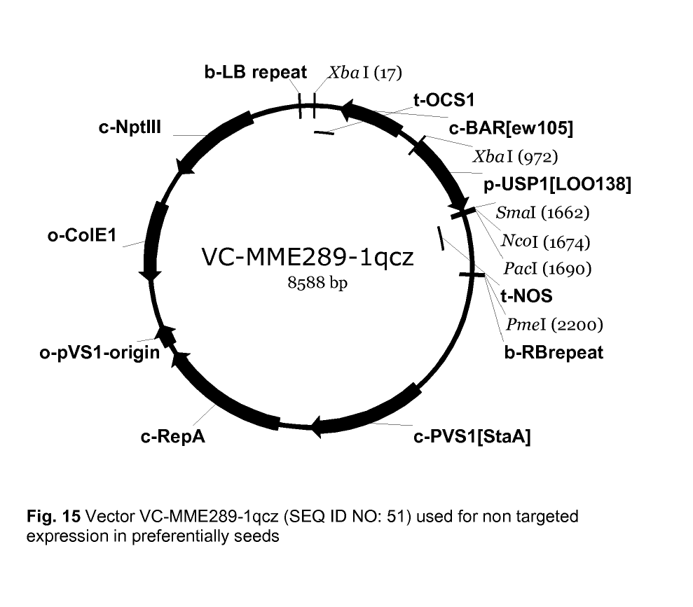

FIG. 15. Vector VC-MME289-1 qcz (SEQ ID NO: 51) used for non targeted expression in preferentially seeds.

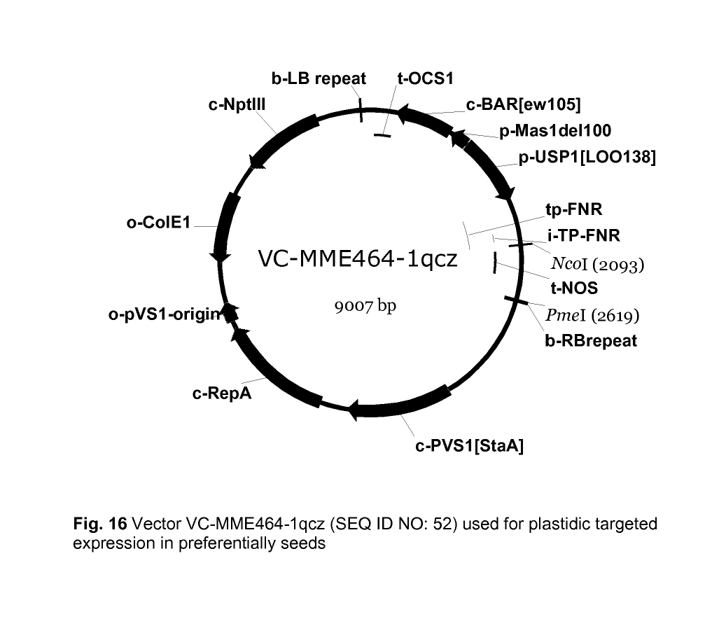

FIG. 16. Vector VC-MME464-1 qcz (SEQ ID NO: 52) used for plastidic targeted expression in preferentially seeds.

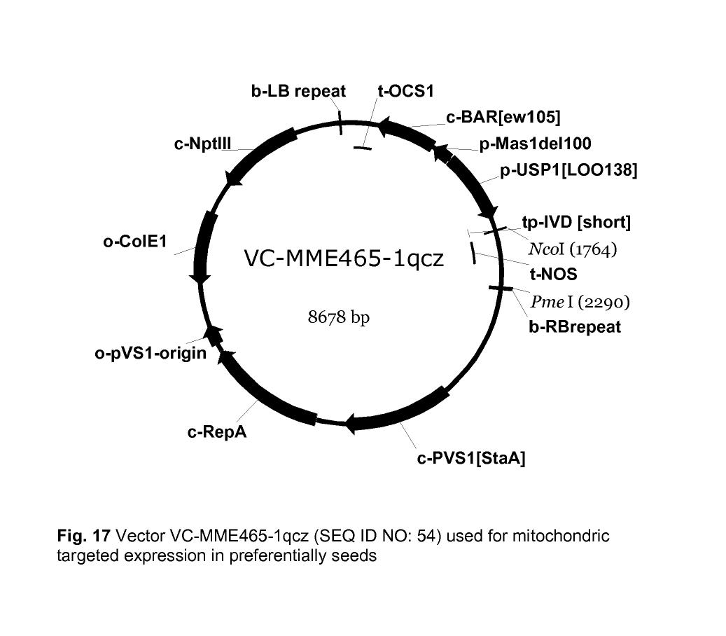

FIG. 17. Vector VC-MME465-1 qcz (SEQ ID NO: 54) used for mitochondric targeted expression in preferentially seeds.

In a further embodiment, the present invention relates in paragraphs [0000.1.8.8.] to [0514.1.8.8.] to a further process for the production of the fine chemical linoleic acid as defined below and corresponding embodiments as described herein as follows.

The present invention relates to a process for the production of fine chemical in a microorganism, a plant cell, a plant or a part thereof. The invention furthermore relates to nucleic acid molecules, polypeptides, nucleic acid constructs, vectors, antibodies, host cells, plant tissue, propagation material, harvested material, plants, microorganisms as well as agricultural compositions and to their use.

Fatty acids are the building blocks of triglycerides, phospholipids, lipids, oils and fats. Some of the fatty acids such as linoleic or linolenic acid are “essential” because the human body is not able to synthesize them but needs them, so humans must ingest them through the diet. The human body can synthesize other fatty acids therefore they are not labeled as “essential”. Nevertheless very often the amount of production of for example fatty acids such as eicosapentaenoic acid (=EPA, C20:5Δ^{5,8,11,14,17})or docosahexaenoic acid (=DHA, C22:6Δ_{4,7,10,13,16,19}) in the body is not sufficient for an optimal body function. Polyunsaturated fatty acids (=PU FA) that mean fatty acids, which have more than 1 double bond in the carbon chain are divided into families depending on where their end-most double bond is located. There are two main subtypes of fatty acids: the omega-3 and omega-6 fatty acids. The Omega 3′s are those with their endmost double bond 3 carbons from their methyl end. The Omega-6′s are those with their endmost double bond 6 carbons from their methyl end. Linoleic acid (an omega-6) and alpha-linolenic acid (an omega-3) are the only true “essential” fatty acids. Both are used inside the body as starting material to synthesize others such as EPA or DHA.

Fatty acids and triglycerides have numerous applications in the food and feed industry, in cosmetics and in the drug sector. Depending on whether they are free saturated or unsaturated fatty acids or bound , e.g. in form of triglycerides with an increased content of saturated or unsaturated fatty acids, they are suitable for the most varied applications; thus, for example, polyunsaturated fatty acids (=PUFAs) are added to infant formula to increase its nutritional value. The various fatty acids and triglycerides are mainly obtained from microorganisms such as fungi, from animals such as fish or from oil-producing plants including phytoplankton and algae, such as soybean, oilseed rape, sunflower and others, where they are usually obtained in the form of their triacylglycerides.

In principal microorganisms such as Mortierella or oil producing plants such as soybean, rapeseed or sunflower, or algae such as Crytocodinium or Phaeodactylum are common sources for oils containing PUFAs, where they are usually obtained in the form of their triacylglycerides. Alternatively, they are obtained advantageously from animals, such as fish. The free fatty acids are prepared advantageously by hydrolysis with a strong base such as potassium or sodium hydroxide. Higher poly unsaturated fatty acids such as DHA, EPA, ARA, Di-homo-γ-linoleic acid (C20:3Δ^{8,11,14}) or Docosapentaenoic acid (=DPA, C22:5Δ^{7,10,13,16,19})are rare or not produced by oil producing plants such as soybean, rapeseed, safflower or sunflower. Natural sources for said fatty acids are fish for example herring, salmon, sardine, redfish, eel, carp, trout, halibut, mackerel, pike-perch or tuna, or algae.

Whether oils with unsaturated or with saturated fatty acids are preferred depends on the intended purpose; thus, for example, lipids with unsaturated fatty acids, specifically polyunsaturated fatty acids, are preferred in human nutrition since they have a positive effect on the cholesterol level in the blood and thus on the possibility of heart disease. They are used in a variety of dietetic foodstuffs or medicaments. In addition PUFAs are commonly used in food, feed and in the cosmetic industry. Polyunsaturated ω-3- and/or ω-6-fatty acids are an important part of animal feed and human food. Because of the common composition of human food polyunsaturated ω-3-fatty acids, which are an essential component of fish oil, should be added to the food to increase the nutritional value of the food; thus, for example, polyunsaturated fatty acids such as DHA or EPA are added as mentioned above to infant formula to increase its nutritional value. The true essential fatty acids linoleic and linolenic fatty acid have a lot of positive effects in the human body such as a positive effect on healthy heart, arteries and skin. They bring for example relieve from eczema, diabetic neuropathy or PMS and cyclical breast pain.

Polyunsaturated ω-3- and ω-6-fatty acids are for example precursor of a family of paracrine hormones called eicosanoids such as prostaglandins which are products of the metabolism of dihomo-γ-linoleic acid, ARA or EPA. Eicosanoids are involved in the regulation of lipolysis, the initiation of inflammatory responses, the regulation of blood circulation and pressure and other central functions of the body. Eicosanoids comprise prostaglandins, leukotrienes, thromboxanes, and prostacyclins. ω-3-fatty acids seem to prevent artherosclerosis and cardiovascular diseases primarily by regulating the levels of different eicosanoids. Other Eicosanoids are the thromboxanes and leukotrienes, which are products of the metabolism of ARA or EPA.

On account of their positive properties there has been no shortage of attempts in the past to make available genes which participate in the synthesis of fatty acids or triglycerides for the production of oils in various organisms having a modified content of unsaturated fatty acids.

Methods of recombinant DNA technology have also been used for some years to improve the oil content in microorganisms or plants by amplifying individual fatty acid biosynthesis genes and investigating the effect on fatty acid production. For example in WO 91/13972 a Δ-9-desaturase is described, which is involved in the synthesis of polyunsaturated fatty acids. In WO 93/11245 a A-15-desaturase and in WO 94/11516 a Δ-12-desaturase is claimed. Other desaturases are described, for example, in WO 95/18222 or Huang et al., Lipids 34, 649 (1999). To date, however, the various desaturases have been only inadequately characterized biochemically since the enzymes in the form of membrane-bound proteins are isolable and characterizable only with very great difficulty (Wang et al., Plant Physiol. Biochem. 26, 777 (1988)). Generally, membrane-bound desaturases are characterized by introduction into a suitable organism, which is then investigated for enzyme activity by means of analysis of starting materials and products. With regard to the effectiveness of the expression of desaturases and their effect on the formation of polyunsaturated fatty acids it may be noted that through expression of desaturases and elongases as described to date only low contents of poly-unsaturated fatty acids/lipids have been achieved. Therefore, an alternative and more effective pathway with higher product yield is desirable. Beside the desaturases mentioned above several other genes have been described to modify the accumulation of fatty acid, for example in WO 2007/087815.

As described above, the essential fatty acids are necessary for humans and many mammals, for example for livestock. In a study of middle-aged men disclosed by Finnish researchers (International Journal of Cancer, Sep. 1, 2004), high intake of linoleic acid seemed to lower the risk of prostate and other cancers. In another publication the positive influence on stroke is disclosed (Umemura et al., Stroke 33, 2086 (2002)).

Improving the quality of foodstuffs and animal feeds is an important task of the food-and-feed industry. This is necessary since, for example, certain fatty acids E, which occur in plants are limited with regard to the supply of mammals. Especially advantageous for the quality of foodstuffs and animal feeds is an as balanced as possible fatty acid profile since a great excess of of certain fatty acids like omega-3-fatty acids above a specific concentration in the food has no further positive effect unless the omega-3-fatty acid content is in balance to the omega-6-fatty acid content of the diet. A further increase in quality is only possible via addition of further fatty acids, which are limiting under these conditions. The targeted addition of the limiting fatty acid in form of synthetic products must be carried out with extreme caution in order to avoid fatty acid imbalance.

To ensure a high quality of foods and animal feeds, it is therefore necessary to add a plurality of fatty acids in a balanced manner to suit the respective organism. Accordingly, there is still a great demand for new and more suitable genes, which encode enzymes or regulators, which participate in the biosynthesis of fatty acids and make it possible to produce certain fatty acids specifically on an industrial scale without unwanted byproducts being formed. In the selection of genes for biosynthesis or regulation two characteristics above all are particularly important. On the one hand, there is as ever a need for improved processes for obtaining the highest possible contents of fatty acids and on the other hand as less as possible byproducts should be produced in the production process.

It is an object of the present invention to develop an inexpensive process for the synthesis of linoleic acid. Linoleic acid is one of the fatty acids , which are most frequently limiting.

It was now found that this object is achieved by providing the process according to the invention described herein and the embodiments characterized herein as well as in the claims.

Accordingly, in a first embodiment, the invention relates to a process for the production of at least one fine chemical selected from the group consisting of: linoleic acid, or, in other words, of the “fine chemical” or “fine chemical of the invention”.

The terms “fine chemical of the invention”, “fine chemical” or “the fine chemical” are used herein equally and relate in context of the paragraphs or sections [0014.1.8.8] to [0514.1.8.8] essentially to the metabolite or the metabolites indicated in column 7, application no. 8 of Tables I, II and IV in the respective line.

Further, the term “in context of any of the paragraphs [0014.1.8.8] to [0514.1.8.8]” as used herein means that for any of said paragraphs [0014.1.8.8] to [0514.1.8.8] the term “the fine chemical” is understood to follow the definition of paragraphs or sections [0014.1.8.8] and [0015.1.8.8], independently whether it refers to any other paragraph or not and whether the reference recites the term “fine chemical” in an other context.

Thus, in cases where one or more paragraphs or sections are incorporated by reference into any of the present paragraphs [0014.1.8.8] to [0514.1.8.8], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to the definition of paragraph [0014.1.8.8] and [0015.1.8.8].

Accordingly, in one embodiment, the term “fine chemical of the invention”, “fine chemical” or “the fine chemical” as used herein (alone or in combination, like in FCRP) means “linoleic acid” in context of the nucleic acid or polypeptide sequences listed in the respective same line of any one of Tables I to IV of application no. 8 and indicating in column 7 the metabolite “linoleic acid”. In one embodiment, the term linoleic acid or the term “fine chemical” mean in context of the paragraphs or sections [0014.1.8.8] to [0514.1.8.8] at least one chemical compound with an activity of the above mentioned linoleic acid, respectively.

Accordingly, in one embodiment, the terms “fine chemical of the invention”, “fine chemical” or “the fine chemical” mean in context of any of the paragraphs [0014.1.8.8] to [0514.1.8.8] linoleic acid, its tryglycerides, lipids, oils and/or fats in free form or bound form. In a preferred embodiment, the term “the fine chemical” means linoleic acid in free form or e.g. in form of its tryglycerides, lipids, oils and/or fats. In a preferred embodiment “linoleic acid” means linoleic acid in free form.

In one embodiment, the term “the fine chemical” or “the respective fine chemical” mean in context of any of the paragraphs [0014.1.8.8] to [0514.1.8.8] linoleic acid and/or tryglycerides, lipids, oils and/or fats of linoleic acid, and/or its salts, ester, thioester or linoleic acid in free form or bound to other compounds such as triglycerides, glycolipids, phospholipids etc.ln a preferred embodiment, the term “the fine chemical” means linoleic acid., in free form or its salts or bound to triglycerides. Triglycerides, lipids, oils, fats or lipid mixture thereof shall mean any triglyceride, lipid, oil and/or fat containing any bound or free linoleic acid for example sphingolipids, phosphoglycerides, lipids, glycolipids such as glycosphingolipids, phospholipids such as phosphatidylethanolamine, phosphatidylcholine, phosphatidylserine, phosphatidylglycerol, phosphatidylinositol or diphosphatidylglycerol, or as monoacylglyceride, diacylglyceride or triacylglyceride or other fatty acid esters such as acetyl-Coenzym A thioester, which contain further saturated or unsaturated fatty acids in the fatty acid molecule.

In one embodiment, the term “the fine chemical” and the term “the respective fine chemical” mean at least one chemical compound with an activity of the above-mentioned fine chemical. Further, the term “in context of any of the paragraphs [0014.1.8.8] to [0514.1.8.8]” as used herein means that for any of said paragraphs [0014.1.8.8] to [0514.1.8.8] the term “the fine chemical” is understood to follow the definition of section [0014.1.8.8] or section [0015.1.8.8], independently whether it refers to any other paragraph or not and whether the reference recites the term “fine chemical” in an other context.

Thus, in cases where one or more paragraphs or sections are incorporated by reference into any of the present paragraphs [0014.1.8.8] to [0514.1.8.8], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to this definition of this paragraph [0015.1.8.8].

Further, the term “fine chemicals” as used herein relates to compositions comprising said fine chemical(s), i.e. comprising linoleic acid, respectively.

Accordingly, the present invention relates to a process for the production of linoleic acid, which comprises
(a) increasing or generating one or more activities selected from the group consisting of chlorophyllase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of At1g19800-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of At2g45420-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of CCAAT-binding transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of transcriptional regulator protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of beta-hydroxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of ThiF family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of recombinase A in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of membrane transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of b0801-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of 3-oxoacyl-[acyl-carrier-protein] synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of outer membrane receptor protein precursor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of b1330-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of lipoprotein precursor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of b1470-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of Fe—S subunit of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of acid shock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of fumarate hydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of electron transport complex protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of b1670-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of b1837-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of b2032-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of colanic acid biosynthesis protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of b2099-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of b2107-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of b2399-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of ethanolamine utilization protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of b2474-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of b2613-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of b2682-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of flavodoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of AAS bifunctional protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of b2849-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of xanthine dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of b2909-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of phosphotriesterase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of sn-glycerol-3-phosphate transport system permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of threonine efflux protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of 3-ketoacyl-CoA thiolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of GM02LC38418-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of sll1032-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of phosphoribosylformyl glycinamidine synthase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of CTP synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of carbamoyl-phosphate synthase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of coproporphyrinogen III oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of cation-transporting ATPase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of tryptophan synthase alpha chain in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of aromatic acid decarboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of phosphate import ATP-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of gamma-glutamyltranspeptidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of arginine decarboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of fatty acid desaturase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of threonine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of TTC0490-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of metal-dependent hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of multiple antibiotic resistance protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of glycin cleavage system protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of aspartate-semialdehyde dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of ydr507c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of YGR104C-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of DnaJ-like chaperone in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of aconitate hydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of molecular chaperone in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of ATPase epsilon subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating one or more activities selected from the group consisting of glucose-6-phosphate 1-dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

An embodiment of the present invention relates to a process for the production of linoleic acid, which comprises
(a1) increasing or generating the activity of a polypeptide comprising a polypeptide as depicted in a respective line in column 5 or 8 of Table II, application no. 8, or a homolog or a fragment thereof, a consensus sequence or at least one polypeptide motif as depicted in the respective line in column 8 of Table IV, application no. 8; or
(a2) increasing or generating the activity of an expression product of one or more nucleic acid molecule(s) comprising a polynucleotide as depicted in the respective line in column 5 or 8 of Table I, application no. 8, preferably the coding region thereof, or a homolog or a fragment thereof; or
(a3) increasing or generating the activity of a functional equivalent of
(a1) or
(a2); in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of linoleic acid or a composition comprising linoleic acid in said non-human organism or in the culture medium surrounding said non-human organism.

Accordingly, the increase or generation of one or more said activities is for example conferred by one or more expression products of said nucleic acid molecule, e.g. proteins. Accordingly, in the present invention described above, the increase or generation of one or more said activities is for example conferred by one or more protein(s) each comprising a polypeptide selected from the group as depicted in Table II, application no. 8, column 5 and 8, or a homolog or a fragment thereof.

The process of the invention comprises in one embodiment the following steps:
(i) increasing or generating of the expression of; and/or
(ii) increasing or generating the expression of an expression product of; and/or
(iii) increasing or generating one or more activities of an expression product encoded by;
   at least one nucleic acid molecule (in the following “Fine Chemical Related Protein (FCRP)”encoding gene or “FCRP”-gene) comprising a nucleic acid molecule selected from the group consisting of:
(a) a nucleic acid molecule encoding the polypeptide shown in column 5 or 8 of Table II, preferably Table II B, application no. 8, or a homolog or a fragment thereof;
(b) a nucleic acid molecule shown in column 5 or 8 of Table I, preferably Table I B, application no. 8, or a homolog or a fragment thereof (preferably the coding region thereof);
(c) a nucleic acid molecule, which, as a result of the degeneracy of the genetic code, can be derived from a polypeptide sequence depicted in column 5 or 8 of Table II, preferably Table II B, application no. 8;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 8, or the coding region thereof;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d),
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 8;
(i) a nucleic acid molecule encoding a polypeptide having the activity represented by a respective protein as depicted in column 5 of Table II, application no. 8;
(j) a nucleic acid molecule which comprises a polynucleotide, which is obtained by amplifying a cDNA library or a genomic library using the primers as depicted in column 8 of Table III, application no. 8; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

The term “increasing or generating of the expression of” may also be interchanged by the term “overexpressing”.

In a preferred embodiment thereof said nucleic acid molecule encodes a polypeptide which has the activity of the polypeptide represented by a protein comprising a polypeptide as depicted in the corresponding hit in column 5 of Table II, application no. 8. In another preferred embodiment thereof said nucleic acid molecule confers a generation or an increase of the respective fine chemical in a non-human organism or a part thereof as compared to a corresponding wild-type non-human organism or part thereof.

In another preferred embodiment thereof said nucleic acid molecule encodes a polypeptide which has the activity of the polypeptide represented by a protein as depicted in the corresponding hit in column 5 of Table II, application no. 8, and confers a generation or an increase of the respective fine chemical in a non-human organism or a part thereof as compared to a corresponding wild-type non-human organism or part thereof.

Accordingly, the genes of the present invention or used in accordance with the present invention, which respectively encode a protein having an activity of 3-ketoacyl-CoA thiolase, 3-oxoacyl-[acyl-carrier-protein] synthase, AAS bifunctional protein, ABC transporter permease protein, acid shock protein, aconitate hydratase, arginine decarboxylase, aromatic acid decarboxylase, aspartate-semialdehyde dehydrogenase, At1g19800-protein, At2g45420-protein, ATPase epsilon subunit, b0801-protein, b1330-protein, b1470-protein, b1670-protein, b1837-protein, b2032-protein, b2099-protein, b2107-protein, b2399-protein, b2474-protein, b2613-protein, b2682-protein, b2849-protein, b2909-protein, beta-hydroxylase, carbamoylphosphate synthase subunit, cation-transporting ATPase, CCAAT-binding transcription factor, chlorophyllase, colanic acid biosynthesis protein, coproporphyrinogen III oxidase, CTP synthetase, DnaJ-like chaperone, electron transport complex protein, ethanolamine utilization protein, fatty acid desaturase, Fe—S subunit of oxidoreductase, flavodoxin, fumarate hydratase, gamma-glutamyltranspeptidase, glucose-6-phosphate 1-dehydrogenase, glutaredoxin, glycin cleavage system protein, GM02LC38418-protein, heat shock transcription factor, hydrolase, lipoprotein precursor, membrane transport protein, metal-dependent hydrolase, molecular chaperone, monothiol glutaredoxin, multiple antibiotic resistance protein, outer membrane receptor protein precursor, oxidoreductase, phosphate import ATP-binding protein, phosphoribosylformyl glycinamidine synthase subunit, phosphotriesterase, protein kinase, protein phosphatase, pyruvate kinase, recombinase A, sll1032-protein, sn-glycerol-3-phosphate transport system permease protein, ThiF family protein, threonine dehydratase, threonine efflux protein, transcription factor, transcriptional regulator protein, tryptophan synthase alpha chain, TTC0490-protein, xanthine dehydrogenase, ydr507c-protein, or YGR104C-protein, which respectively encode a protein comprising a polypeptide encoded by a nucleic acid sequence as shown in Table I, application no. 8, column 5 or 8, (preferably the coding region thereof), or a homolog or a fragment thereof, which respectively encode a protein comprising a polypeptide as depicted in Table II, application no. 8, column 5 or 8, or a homolg or a fragment thereof, and/or which respectively can be amplified with the primer set shown in Table III, application no. 8, column 8, are also referred to as “FCRP genes”.

Proteins or polypeptides encoded by “FCRP-genes” are referred to as “Fine Chemical Related Proteins” or “FCRP”. For the purposes of the description of the present invention, the respective protein having an activity of 3-ketoacyl-CoA thiolase, 3-oxoacyl-[acyl-carrierprotein] synthase, AAS bifunctional protein, ABC transporter permease protein, acid shock protein, aconitate hydratase, arginine decarboxylase, aromatic acid decarboxylase, aspartate-semialdehyde dehydrogenase, At1g19800-protein, At2g45420-protein, ATPase epsilon subunit, b0801-protein, b1330-protein, b1470-protein, b1670-protein, b1837-protein, b2032-protein, b2099-protein, b2107-protein, b2399-protein, b2474-protein, b2613-protein, b2682-protein, b2849-protein, b2909-protein, beta-hydroxylase, carbamoyl-phosphate synthase subunit, cation-transporting ATPase, CCAAT-binding transcription factor, chlorophyllase, colanic acid biosynthesis protein, coproporphyrinogen III oxidase, CTP synthetase, DnaJ-like chaperone, electron transport complex protein, ethanolamine utilization protein, fatty acid desaturase, Fe—S subunit of oxidoreductase, flavodoxin, fumarate hydratase, gamma-glutamyltranspeptidase, glucose-6-phosphate 1-dehydrogenase, glutaredoxin, glycin cleavage system protein, GM02LC38418-protein, heat shock transcription factor, hydrolase, lipoprotein precursor, membrane transport protein, metal-dependent hydrolase, molecular chaperone, monothiol glutaredoxin, multiple antibiotic resistance protein, outer membrane receptor protein precursor, oxidoreductase, phosphate import ATP-binding protein, phosphoribosylformyl glycinamidine synthase subunit, phosphotriesterase, protein kinase, protein phosphatase, pyruvate kinase, recombinase A, sll1032-protein, sn-glycerol-3-phosphate transport system permease protein, ThiF family protein, threonine dehydratase, threonine efflux protein, transcription factor, transcriptional regulator protein, tryptophan synthase alpha chain, TTC0490-protein, xanthine dehydrogenase, ydr507c-protein, or YGR104C-protein, the respective protein comprising a polypeptide encoded by one or more respective nucleic acid sequences as shown in Table I, application no. 8, column 5 or 8, (preferably the coding region thereof), or a homolog or fragment thereof, the respective protein comprising a respective polypeptide as depicted in Table II, application no. 8, column 5 or 8, or a homolog or fragment thereof, the respective protein comprising a sequence corresponding to the consensus sequence as shown in Table IV, application no. 8, column 8, and/or the respective protein comprising at least one polypeptide motif as shown in Table IV, application no. 8, column 8 is also referred to as Fine Chemical Related Protein” or “FCRP”.

Thus, in one embodiment, the present invention provides a process of the production of linoleic acid, by increasing or generating one or more activities, especially selected from the group consisting of 3-ketoacyl-CoA thiolase, 3-oxoacyl-[acyl-carrier-protein] synthase, AAS bifunctional protein, ABC transporter permease protein, acid shock protein, aconitate hydratase, arginine decarboxylase, aromatic acid decarboxylase, aspartate-semialdehyde dehydrogenase, At1g19800-protein, At2g45420-protein, ATPase epsilon subunit, b0801-protein, b1330-protein, b1470-protein, b1670-protein, b1837-protein, b2032-protein, b2099-protein, b2107-protein, b2399-protein, b2474-protein, b2613-protein, b2682-protein, b2849-protein, b2909-protein, beta-hydroxylase, carbamoyl-phosphate synthase subunit, cationtransporting ATPase, CCAAT-binding transcription factor, chlorophyllase, colanic acid biosynthesis protein, coproporphyrinogen III oxidase, CTP synthetase, DnaJ-like chaperone, electron transport complex protein, ethanolamine utilization protein, fatty acid desaturase, Fe—S subunit of oxidoreductase, flavodoxin, fumarate hydratase, gamma-glutamyltranspeptidase, glucose-6-phosphate 1-dehydrogenase, glutaredoxin, glycin cleavage system protein, GM02LC38418-protein, heat shock transcription factor, hydrolase, lipoprotein precursor, membrane transport protein, metal-dependent hydrolase, molecular chaperone, monothiol glutaredoxin, multiple antibiotic resistance protein, outer membrane receptor protein precursor, oxidoreductase, phosphate import ATP-binding protein, phosphoribosylformyl glycinamidine synthase subunit, phosphotriesterase, protein kinase, protein phosphatase, pyruvate kinase, recombinase A, sll1032-protein, sn-glycerol-3-phosphate transport system permease protein, ThiF family protein, threonine dehydratase, threonine efflux protein, transcription factor, transcriptional regulator protein, tryptophan synthase alpha chain, TTC0490-protein, xanthine dehydrogenase, ydr507c-protein, and YGR104C-protein, which is conferred by one or more FCRPs or the gene product of one or more FCRP-genes, for example by the gene product of a nucleic acid sequences comprising a polynucleotide selected from the group as shown in Table I, application no. 8, column 5 or 8, (preferably by the coding region thereof), or a homolog or a fragment thereof, e.g. or by one or more proteins each comprising a polypeptide encoded by one or more nucleic acid sequences selected from the group as shown in Table I, application no. 8, column 5 or 8, (preferably by the coding region thereof), or a homolog or a fragment thereof, or by one or more protein(s) each comprising a polypeptide selected from the group as depicted in Table II, application no. 8, column 5 and 8, or a homolog thereof, or a protein comprising a sequence corresponding to the consensus sequence or comprising at least one polypeptide motif as shown in Table IV, application no. 8, column 8.

As mentioned, the process for the production of the fine chemical according to the present invention, in particular showing a generation or an increase of the respective fine chemical in a non-human organism or a part thereof as compared to a corresponding wild-type non-human organism or part thereof, can be mediated by one or more FCRP-genes or FCPRs.

In an embodiment, the process comprises increasing or generating the activity of one or more polypeptides having said activity, e.g.

by generating or increasing the amount and/or specific activity in the cell or a compartment of a cell of one of more FCRP, especially selected from the group consisting of 3-ketoacyl-CoA thiolase, 3-oxoacyl-[acyl-carrier-protein] synthase, AAS bifunctional protein, ABC transporter permease protein, acid shock protein, aconitate hydratase, arginine decarboxylase, aromatic acid decarboxylase, aspartate-semialdehyde dehydrogenase, At1g19800-protein, At2g45420-protein, ATPase epsilon subunit, b0801-protein, b1330-protein, b1470-protein, b1670-protein, b1837-protein, b2032-protein, b2099-protein, b2107-protein, b2399-protein, b2474-protein, b2613-protein, b2682-protein, b2849-protein, b2909-protein, beta-hydroxylase, carbamoylphosphate synthase subunit, cation-transporting ATPase, CCAAT-binding transcription factor, chlorophyllase, colanic acid biosynthesis protein, coproporphyrinogen III oxidase, CTP synthetase, DnaJ-like chaperone, electron transport complex protein, ethanolamine utilization protein, fatty acid desaturase, Fe—S subunit of oxidoreductase, flavodoxin, fumarate hydratase, gamma-glutamyltranspeptidase, glucose-6-phosphate 1-dehydrogenase, glutaredoxin, glycin cleavage system protein, GM02LC38418-protein, heat shock transcription factor, hydrolase, lipoprotein precursor, membrane transport protein, metal-dependent hydrolase, molecular chaperone, monothiol glutaredoxin, multiple antibiotic resistance protein, outer membrane receptor protein precursor, oxidoreductase, phosphate import ATP-binding protein, phosphoribosylformyl glycinamidine synthase subunit, phosphotriesterase, protein kinase, protein phosphatase, pyruvate kinase, recombinase A, sll1032-protein, sn-glycerol-3-phosphate transport system permease protein, ThiF family protein, threonine dehydratase, threonine efflux protein, transcription factor, transcriptional regulator protein, tryptophan synthase alpha chain, TTC0490-protein, xanthine dehydrogenase, ydr507c-protein, and YGR104C-protein, for example of the respective polypeptide as depicted in Table II, application no. 8, column 5 and 8, or a homolog or a fragment thereof, or the respective polypeptide comprising a sequence corresponding to the consensus sequences as shown in Table IV, application no. 8, column 8, or the respective polypeptide comprising at least one polypeptide motif as depicted in Table IV, application no. 8, column 8.

A further embodiment of the present invention relates to a process for the production of linoleic acid, which comprises
(a) increasing or generating the activity of a chlorophyllase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a At1g19800-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a At2g45420-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a CCAAT-binding transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a transcriptional regulator protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a beta-hydroxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a ThiF family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a recombinase A non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a membrane transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a b0801-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a 3-oxoacyl-[acyl-carrier-protein] synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a outer membrane receptor protein precursor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a b1330-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a lipoprotein precursor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a b1470-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a Fe—S subunit of oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a acid shock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a fumarate hydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a electron transport complex protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a b1670-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a b1837-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a b2032-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a colanic acid biosynthesis protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a b2099-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a b2107-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a b2399-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a ethanolamine utilization protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a b2474-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a b2613-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a b2682-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a flavodoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a AAS bifunctional protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a b2849-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a xanthine dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a b2909-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a phosphotriesterase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a sn-glycerol-3-phosphate transport system permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a threonine efflux protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a 3-ketoacyl-CoA thiolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a GM02LC38418-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a sll1032-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a phosphoribosylformyl glycinamidine synthase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a CTP synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a carbamoyl-phosphate synthase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a coproporphyrinogen III oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a cation-transporting ATPase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a tryptophan synthase alpha chain non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a aromatic acid decarboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a phosphate import ATP-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a gamma-glutamyltranspeptidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a arginine decarboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a fatty acid desaturase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a threonine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a TTC0490-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a metal-dependent hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a multiple antibiotic resistance protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a glycin cleavage system protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a aspartate-semialdehyde dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a ydr507c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a YGR104C-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a DnaJ-like chaperone non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a aconitate hydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a molecular chaperone non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a ATPase epsilon subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a glucose-6-phosphate 1-dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating the activity of a polypeptide comprising a polypeptide as depicted in the respective line in column 5 or 8 of Table II, application no. 8, or a homolog or a fragment thereof, a consensus sequence or at least one polypeptide motif as depicted in the respective line in column 8 of Table IV, application no. 8; or increasing or generating the activity of an expression product of one or more nucleic acid molecule(s) comprising a polynucleotide as depicted in the respective line in column 5 or 8 of Table I, application no. 8, preferably the coding region thereof, or a homolog or a fragment thereof; non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of linoleic acid, or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of chlorophyllase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of At1g19800-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of At2g45420-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of CCAAT-binding transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of transcriptional regulator protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of betahydroxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of ThiF family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of recombinase A in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of membrane transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of b0801-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of 3-oxoacyl-[acyl-carrier-protein] synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of outer membrane receptor protein precursor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of b1330-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of lipoprotein precursor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of b1470-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of Fe—S subunit of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of acid shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of fumarate hydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of electron transport complex protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of b1670-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of b1837-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of b2032-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of colanic acid biosynthesis protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of b2099-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of b2107-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of b2399-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of ethanolamine utilization protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of b2474-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of b2613-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of b2682-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of flavodoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of AAS bifunctional protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of b2849-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of xanthine dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of b2909-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of phosphotriesterase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of sn-glycerol-3-phosphate transport system permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of threonine efflux protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of 3-ketoacyl-CoA thiolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of GM02LC38418-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of sll1032-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of phosphoribosylformyl glycinamidine synthase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of CTP synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of carbamoyl-phosphate synthase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of coproporphyrinogen III oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of cation-transporting ATPase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of tryptophan synthase alpha chain in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of aromatic acid decarboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of phosphate import ATP-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of gamma-glutamyltranspeptidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of arginine decarboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of fatty acid desaturase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of threonine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of TTC0490-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of metal-dependent hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of multiple antibiotic resistance protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of glycin cleavage system protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of aspartate-semialdehyde dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of ydr507c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of YGR104C-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of DnaJ-like chaperone in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of aconitate hydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of molecular chaperone in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of ATPase epsilon subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a) increasing or generating one or more activities selected from the group consisting of glucose-6-phosphate 1-dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

A further embodiment of the present invention relates to a process for the production of linoleic acid, which comprises
(a1) increasing or generating the activity of a polypeptide comprising a polypeptide as depicted in the respective line in column 5 or 8 of Table II, application no. 8, or a homolog or fragment thereof, a consensus sequence or at least one polypeptide motif as depicted in column 8 of Table IV, application no. 8; or
increasing or generating the activity of an expression product of one or more nucleic acid molecule(s) comprising a polynucleotide as depicted in the respective line in column 5 or 8 of Table I, application no. 8, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 8, or a homolog or a fragment thereof, a consensus sequence or at least one polypeptide motif as depicted in the respective line in column 8 of Table IV, application no. 8, which is joined to a transit peptide; or
increasing or generating the activity of an expression product of one or more nucleic acid molecule(s) comprising a polynucleotide as depicted in the respective line in column 5 or 8 of Table I, application no. 8, preferably the coding region thereof, or a homolog or a fragment thereof, which is joined to a nucleic acid sequence encoding an organelle localization sequence, preferably a plastid or a mitochondrion localization sequence, especially a plastid localization sequence;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 8, or a homolog or a fragment thereof, a consensus sequence or at least one polypeptide motif as depicted in the respective line in column 8 of Table IV, application no. 8; or increasing or generating the activity of an expression product of one or more nucleic acid molecule(s) comprising a polynucleotide as depicted in the respective line in column 5 or 8 of Table I, application no. 8, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of linoleic acid, or a composition comprising linoleic acid in said non-human organism or in the culture medium surrounding said non-human organism.

Accordingly, the present invention relates to a process for the production of linoleic acid, which comprises
(a) increasing or generating the activity of a chlorophyllase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a At1g19800-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a At2g45420-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a CCAAT-binding transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a transcriptional regulator protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a beta-hydroxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a ThiF family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a recombinase A in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a membrane transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a b0801-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a 3-oxoacyl-[acyl-carrier-protein] synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a outer membrane receptor protein precursor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a b1330-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a lipoprotein precursor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a b1470-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a Fe—S subunit of oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a acid shock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a fumarate hydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a electron transport complex protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a b1670-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a b1837-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a b2032-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a colanic acid biosynthesis protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a b2099-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a b2107-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a b2399-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a ethanolamine utilization protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a b2474-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a b2613-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a b2682-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a flavodoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a AAS bifunctional protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a b2849-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a xanthine dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a b2909-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a phosphotriesterase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a sn-glycerol-3-phosphate transport system permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a threonine efflux protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a 3-ketoacyl-CoA thiolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a GM02LC38418-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a sll1032-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a phosphoribosylformyl glycinamidine synthase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a CTP synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a carbamoyl-phosphate synthase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a coproporphyrinogen III oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a cation-transporting ATPase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a tryptophan synthase alpha chain in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a aromatic acid decarboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a phosphate import ATP-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a gamma-glutamyltranspeptidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a arginine decarboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a fatty acid desaturase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a threonine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a TTC0490-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a metal-dependent hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a multiple antibiotic resistance protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a glycin cleavage system protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a aspartate-semialdehyde dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a ydr507c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a YGR104C-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a DnaJ-like chaperone in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a aconitate hydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a molecular chaperone in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a ATPase epsilon subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a glucose-6-phosphate 1-dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Accordingly, the present invention relates to a process for the production of linoleic acid, which comprises
(a) increasing or generating the activity of a polypeptide comprising a polypeptide as depicted in the respective line in column 5 or 8 of Table II, application no. 8, or a homolog or a fragment thereof, a consensus sequence or at least one polypeptide motif as depicted in the respective line in column 8 of Table IV, application no. 8; or
increasing or generating the activity of an expression product of one or more nucleic acid molecule(s) comprising a polynucleotide as depicted in the respective line in column 5 or 8 of Table I, application no. 8, preferably the coding region thereof, or a homolog or a fragment thereof;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of linoleic acid, or a composition comprising linoleic acid in said non-human organism or in the culture medium surrounding said non-human organism.

In a further embodiment the activity of the polypeptide comprising a polypeptide as depicted in the respective line in column 5 or 8 of Table II, application no. 8, or a homolog or a fragment thereof, a consensus sequence or at least one polypeptide motif as depicted in the respective line in column 8 of Table IV, application no. 8, is increased or generated non-targeted in the above-mentioned process in a microorganism or plant or a part thereof.

In a further embodiment said polypeptide has the activity of the respective polypeptide represented by a protein comprising a polypeptide as depicted in the respective line in column 5 of Table II, application no. 8.

In a further embodiment the activity of the expression product of one or more nucleic acid molecule(s) comprising a polynucleotide as depicted in the respective line in column 5 or 8 of Table I, application no. 8, preferably the coding region thereof, or a homolog or a fragment thereof, is increased or generated non-targeted in the above-mentioned process in a microorganism or plant or a part thereof.

In a further embodiment the activity of the polypeptide comprising a polypeptide as depicted in the respective line in column 5 or 8 of Table II, application no. 8, or a homolog or a fragment thereof, a consensus sequence or at least one polypeptide motif as depicted in the respective line in column 8 of Table IV, application no. 8, is increased or generated in the above-mentioned process in an organelle preferably in plastids or mitochondria, especially in plastids, of a microorganism or plant.

In a further embodiment said polypeptide has the activity of the respective polypeptide represented by a protein comprising a polypeptide as depicted in the respective line in column 5 of Table II, application no. 8.

In a further embodiment the activity of the expression product of one or more nucleic acid molecule(s) comprising a polynucleotide as depicted in the respective line in column 5 or 8 of Table I, application no. 8, preferably the coding region thereof, or a homolog or a fragment thereof, is increased or generated in the above-mentioned process in an organelle, preferably in plasids or mitochondria, especially in plastids, of a microorganism or plant.

In a further embodiment the activity of the polypeptide comprising a polypeptide as depicted in the respective line in column 5 or 8 of Table II, application no. 8, or a homolog or a fragment thereof, a consensus sequence or at least one polypeptide motif as depicted in the respective line in column 8 of Table IV, application no. 8, is increased or generated in the above-mentioned process in the cyctosol of a cell, of a microorganism or plant.

In a further embodiment said polypeptide has the activity of the respective polypeptide represented by a protein comprising a polypeptide as depicted in the respective line in column 5 of Table II, application no. 8.

In a further embodiment the activity of the expression product of one or more nucleic acid molecule(s) comprising a polynucleotide as depicted in the respective line in column 5 or 8 of Table I, application no. 8, preferably the coding region thereof, or a homolog or a fragment thereof, is increased or generated in the above-mentioned process in the cytosol of a cell, of a microorganism or plant.

In a further embodiment of the present invention the process further comprises the step of recovering the fine chemical, which is synthesized by the organism from the organism and/or from the culture medium used for the growth or maintenance of the organism.

For the purposes of the present invention, as a rule the plural is intended to encompass the singular and vice versa, unless otherwise specified.

Comprises/comprising and grammatical variations thereof when used in this specification are to be taken to specify the presence of stated features, integers, steps or components or groups thereof, but not to preclude the presence or addition of one or more other features, integers, steps, components or groups thereof.

The term “Table I” used in this specification is to be taken to specify the content of Table I A and Table I B. The term “Table II” used in this specification is to be taken to specify the content of Table II A and Table II B. The term “Table I A” used in this specification is to be taken to specify the content of Table I A. The term “Table I B” used in this specification is to be taken to specify the content of Table I B. The term “Table II A” used in this specification is to be taken to specify the content of Table II A. The term “Table II B” used in this specification is to be taken to specify the content of Table II B. In one preferred embodiment, the term “Table I” means Table I B. In one preferred embodiment, the term “Table II” means Table II B.

In a line of Table I related nucleic acid molecules are listed. In column 3 the locus name, often also referred to as gene name, is given, in column 5 the lead sequence ID No. thereto and in column 8 the sequence ID No. of homologues thereof. In the corresponding line of Table II the respective polypeptides are listed. In column 3 the protein name is given (which is according to the common understanding of the skilled person in the art usually used for the gene as well as the polypeptide and therefore identical with the gene name/locus name), in column 5 the (corresponding) lead sequence ID No. thereto and in column 8 the (corresponding) sequence ID No. of homologues thereof.

In Tables I and II in column 4 information is given from which organism the lead sequence according to column 5 has been identified, in column 7 information is given which fine chemical is generated or increased, and in an especial embodiment in column 6 information is given about non-targeted expression or expression in plastids or mitochondria. Tables III and IV are arranged accordingly whereby in column 8 of Table III primers are listed which can be used to amplify the sequence of the corresponding lead sequence indicated in column 5 of the same line and whereby in column 8 of Table IV consensus and pattern sequences are listed which are shared by the lead sequence as indicated in column 5 of the same line and their homologs listed in the same line in Table II column 8. How the consensus and pattern sequences are determinedis described lateron in the application in more detail.

The terms “increase”, “raise”, “extend”, “enhance”, “improve” and “amplify” as well as the grammatical versions thereof relate to a corresponding change of a property in a non-human organism, a part of an organism such as a tissue, seed, root, leave, flower, pollen etc. or in a cell and are interchangeable. Preferably, the overall activity in the volume is increased or enhanced in cases if the increase or enhancement is related to the increase or enhancement of an activity of a gene product, independent whether the amount of gene product or the specific activity of the gene product or both is increased or enhanced or whether the amount, stability or translation efficacy of the nucleic acid sequence or gene encoding for the gene product is increased or enhanced.

Under “change of a property” it is understood that the activity, expression level or amount of a gene product or the metabolite content is changed in a specific volume relative to a corresponding volume of a control, reference or wild type, including the de novo creation of the activity or expression.

The term “increase” may be directed to a change of said property in the subject of the present invention or only in a part thereof, for example, the change can be found in a compartment of a cell, like an organelle, or in a part of an non-human organism, like plant tissue, plant seed, plant root, pollen, leave, flower etc. but is not detectable in the overall subject, i.e. complete cell or plant, if tested.

Accordingly, the term “increase” means that the specific activity of a polypeptide or the amount of a compound or of a metabolite, e.g. of a polypeptide, a nucleic acid molecule or an encoding mRNA or DNA or the fine chemical, can be increased in a volume.

The term “increase” includes that a compound or an activity is introduced into a cell or a subcellular compartment or organelle de novo or that the compound or the activity has not been detectable before, in other words it is “generated”.

Accordingly, in the following, the term “increasing” also comprises the term “generating” or “stimulating”. The increased activity manifests itself in an increase of the fine chemical.

The terms “wild type”, “control” or “reference” are exchangeable and can be a cell or a part of a non-human organism such as an organelle like a chloroplast or a tissue, or a non-human organism, in particular a microorganism or a plant, which was not modified or treated according to the herein described process according to the invention. Accordingly, the cell or a part of a non-human organism such as an organelle like a chloroplast or a tissue, or an organism, in particular a microorganism or a plant used as wild type, control or reference corresponds to the cell, non-human organism, microorganism, plant, or a part thereof, as much as possible and is in any other property but in the result of the process of the invention as identical to the subject matter of the invention as possible. Thus, the wild type, control or reference is treated identically or as identical as possible, saying that only conditions or properties might be different which do not influence the quality of the tested property.

Preferably, any comparison is carried out under analogous conditions. The term “analogous conditions” means that all conditions such as, for example, culture or growing conditions (such as soil, nutrient, water content of the soil, temperature, humidity or surrounding air), or assay conditions (such as buffer composition, temperature, substrates, pathogen strain, concentrations and the like) are kept identical between the experiments to be compared.

The “reference”, “control” or “wild type” is preferably a subject, e.g. an organelle, a cell, a tissue, an organism, in particular a microorganism or a plant, which was not modified or treated according to the herein described process of the invention and is in any other property as similar to the subject matter of the invention as possible. The reference, control or wild type is in its genome, transcriptome, proteome or metabolome as similar as possible to the subject of the present invention. Preferably, the term “reference-”, “control-” or “wild type-”-organelle, -cell, -tissue or -organism, in particular a microorganism or a plant, relates to an organelle, cell, tissue or organism, in particular a microorganism or a plant, which is nearly genetically identical to the organelle, cell, tissue or organism, in particular a microorganism or plant, or a part thereof, of the present invention, preferably 95%, more preferred are 98%, even more preferred are 99.00%, in particular 99.10%, 99.30%, 99.50%, 99.70%, 99.90%, 99.99%, 99.999% or more. Most preferable the “reference”, “control”, or “wild type” is a subject, e.g. an organelle, a cell, a tissue, an organism, in particular a microorganism or a plant, which is genetically identical to the organism, in particular plant, cell, a tissue or organelle used according to the process of the invention except that the responsible or activity conferring nucleic acid molecules or the gene product encoded by them are amended, manipulated, exchanged or introduced according to the inventive process.

In case, a control, reference or wild type differing from the subject of the present invention only by not being subject of the process of the invention can not be provided, a control, reference or wild type can be a non-human organism in which the cause for the modulation of an activity conferring the generation/increase of the fine chemical or expression of the nucleic acid molecule of the invention as described herein has been switched back or off, e.g. by knocking out the expression of responsible gene product, e.g. by antisense inhibition, by inactivation of an activator or agonist, by activation of an inhibitor or antagonist, by inhibition through adding inhibitory antibodies, by adding active compounds as e.g. hormones, by introducing negative dominant mutants, etc. A gene production can for example be knocked out by introducing inactivating point mutations, which lead to an enzymatic activity inhibition or a destabilization or an inhibition of the ability to bind to cofactors etc.

Accordingly, preferred reference subject is the starting subject of the present process of the invention. Preferably, the reference and the subject matter of the invention are compared after standardization and normalization, e.g. to the amount of total RNA, DNA, or protein or activity or expression of reference genes, like housekeeping genes, such as ubiquitin, actin or ribosomal proteins.

In accordance with the invention, the term “non-human organism” as understood herein relates always to an organism with the exception of a human being, in particular to an animal or a plant or a microorganism. Further, the term “animal” as understood herein relates always to a non-human animal. Preferably the term “non-human organism” shall mean a microorganism, in particular those containing plastids, such as algae, or a plant or the like.

As used herein, “plant” is meant to include not only a whole plant but also a part thereof i.e., one or more cells, tissues, including for example, leaves, stems, shoots, roots, flowers, fruits, seeds and pollen.

The term “organelle” according to the invention shall mean for example “mitochondrion” or “plastid”. The term “plastid” according to the invention is intended to include vahous forms of plastids including proplastids, chloroplasts, chromoplasts, gerontoplasts, leucoplasts, amyloplasts, elaioplasts and etioplasts, preferably chloroplasts. They all have as a common ancestor the aforementioned proplasts.

Unless otherwise specified, the terms “polynucleotides”, “nucleic acid” and “nucleic acid molecule” are interchangeable in the present context. Unless otherwise specified, the terms “peptide”, “polypeptide” and “protein” are interchangeably in the present context. The term “sequence” may relate to polynucleotides, nucleic acids, nucleic acid molecules, peptides, polypeptides and proteins, depending on the context in which the term “sequence” is used. The terms “gene(s)”, “polynucleotide”, “nucleic acid sequence”, “nucleotide sequence”, or “nucleic acid molecule(s)” as used herein refer to a polymeric form of nucleotides of any length, either ribonucleotides or deoxyribonucleotides. The terms refer only to the primary structure of the molecule.

Thus, the terms “gene(s)”, “polynucleotide”, “nucleic acid sequence”, “nucleotide sequence”, or “nucleic acid molecule(s)” as used herein include double- and single-stranded DNA and/or RNA. They also include known types of modifications, for example, methylation, “caps”, substitutions of one or more of the naturally occurring nucleotides with an analog. Preferably, the DNA or RNA sequence comprises a coding sequence encoding the herein defined polypeptide.

A “coding sequence” is a nucleotide sequence, which is transcribed into a RNA, e.g. a regulatory RNA, such as a miRNA, a ta-siRNA, cosuppression molecule, an RNAi, a ribozyme, etc. or preferably into a mRNA which is translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleotide sequences or genomic DNA, while introns may be present as well under certain circumstances. The terms “coding sequence” and “coding region” are interchangeable in the present context.

As used in the present context a nucleic acid molecule may also encompass the untranslated sequence located at the 3′ and/or at the 5′ end of the coding gene region, for example at least 500, preferably 200, especially preferably 100, nucleotides of the sequence upstream of the 5′ end of the coding region and/or at least 100, preferably 50, especially preferably 20, nucleotides of the sequence downstream of the 3′ end of the coding gene region. In the event for example the antisense, RNAi, snRNA, dsRNA, siRNA, miRNA, ta-siRNA, cosuppression molecule, ribozyme etc. technology is used coding regions as well as the 5′- and/or 3′-regions can advantageously be used.

However, it is often advantageous only to choose the coding region of a nucleic acid sequence for cloning and expression purposes. Except as not otherwise specified in the sequence listing according to WI PO Standard 25 for a respective CDS usually the coding region of a nucleic acid molecule is depicted at numeric identifier <222>, whereby the coding region starts at the position given by the first number (given in brackets) and ends at the position given by the second number (given in brackets) and thereafter under numeric identifier <400>the respective sequence is disclosed.

Unless otherwise specified, the term “polypeptide” refers to a polymer of amino acids (amino acid sequence) and does not refer to a specific length of the molecule. Thus, peptides and oligopeptides are included within the definition of polypeptide. This term does also refer to or include post-translational modifications of the polypeptide, for example, glycosylations, acetylations, phosphorylations and the like. Included within the definition are, for example, polypeptides containing one or more analogs of an amino acid (including, for example, unnatural amino acids, etc.), polypeptides with substituted linkages, as well as other modifications known in the art, both naturally occurring and non-naturally occurring. The terms “protein” and “polypeptide” used in this application are interchangeable.

The term “recovering” means the isolation of the fine chemical in different purities, that means on the one hand harvesting of the biological material, which contains the fine chemical without further purification and on the other hand purities of the fine chemical between 5% and 100% purity, preferred purities are in the range of 10% and 99%. In one embodiment, the purities are at least 20%, 30%, 40%, 50%, 60%, 70%, 80%, 90%, 95% or 99%.

A “transit peptide” is an amino acid sequence, whose encoding nucleic acid sequence is translated together with the corresponding structural gene. That means the transit peptide is an integral part of the translated protein and forms an amino terminal extension of the protein. Both are translated as so called “preprotein”. In general the transit peptide is cleaved off from the preprotein during or just after import of the protein into the correct cell organelle such as a plastid or mitochondrion to yield the mature protein. The transit peptide ensures correct localization of the mature protein by facilitating the transport of proteins through intracellular membranes. In principle the nucleic acid sequence encoding a transit peptide can be isolated from every organism such as microorganisms such as algae or plants containing plastids, preferably chloroplasts, or mitochondria. Preferred nucleic acid sequences encoding a transit peptide are derived from a nucleic acid sequence encoding a protein finally resided in the plastid or mitochondria, respectively, and stemming from an organism selected from the group consisting of the genera:

Acetabularia, Arabidopsis, Brassica, Capsicum, Chlamydomonas, Cucurbita, Dunaliella, Euglena, Flaveria, Glycine, Helianthus, Hordeum, Lemna, Lolium, Lycopersion, Malus, Medicago, Mesembryanthemum, Nicotiana, Oenotherea, Oryza, Petunia, Phaseolus, Physcomitrella, Pinus, Pisum, Raphanus, Silene, Sinapis, Solanum, Spinacea, Stevia, Synechococcus, Triticum and Zea.

Advantageously plastidial transit peptides, which are beneficially used in an embodiment of the process of the present invention, are derived from the nucleic acid sequence encoding a protein selected from the group consisting of ribulose bisphosphate carboxylase/oxygenase, 5-enolpyruvyl-shikimate-3-phosphate synthase, acetolactate synthase, chloroplast ribosomal protein CS17, Cs protein, ferredoxin, plastocyanin, ribulose bisphosphate carboxylase activase, tryptophan synthase, acyl carrier protein, plastid chaperonin-60, cytochrome c552, 22-kDA heat shock protein, 33-kDa Oxygen-evolving enhancer protein 1, ATP synthase γ subunit, ATP synthase δ subunit, chlorophyll-a/b-binding proteinII-1, Oxygen-evolving enhancer protein 2, Oxygen-evolving enhancer protein 3, photosystem I: P21, photosystem I: P28, photosystem I: P30, photosystem I: P35, photosystem I: P37, glycerol-3-phosphate acyltransferases, chlorophyll a/b binding protein, CAB2 protein, hydroxymethyl-bilane synthase, pyruvate-orthophosphate dikinase, CAB3 protein, plastid ferritin, ferritin, early light-inducible protein, glutamate-1-semialdehyde aminotransferase, protochlorophyllide reductase, starch-granule-bound amylase synthase, light-harvesting chlorophyll a/b-binding protein of photosystem II, major pollen allergen Lol p 5a, plastid CIpB ATP-dependent protease, superoxide dismutase, ferredoxin NADP oxidoreductase, 28-kDa ribonucleoprotein, 31-kDa ribonucleoprotein, 33-kDa ribonucleoprotein, acetolactate synthase, ATP synthase CFO subunit 1, ATP synthase CFO subunit 2, ATP synthase CFO subunit 3, ATP synthase CFO subunit 4, cytochrome f, ADP-glucose pyrophosphorylase, glutamine synthase, glutamine synthase 2, carbonic anhydrase, GapA protein, heat-shock-protein hsp21, phosphate translocator, plastid CIpA ATP-dependent protease, plastid ribosomal protein CL24, plastid ribosomal protein CL9, plastid ribosomal protein PsCL18, plastid ribosomal protein PsCL25, DAHP synthase, starch phosphorylase, root acyl carrier protein II, betaine-aldehyde dehydrogenase, GapB protein, glutamine synthetase 2, phosphoribulokinase, nitrite reductase, ribosomal protein L12, ribosomal protein L13, ribosomal protein L21, ribosomal protein L35, ribosomal protein L40, triose phosphate-3-phosphoglyerate-phosphate translocator, ferredoxin-dependent glutamate synthase, glyceraldehyde-3-phosphate dehydrogenase, NADP-dependent malic enzyme and NADP-malate dehydrogenase.

More preferred the nucleic acid sequence encoding a plastidal transit peptide is derived from a nucleic acid sequence encoding a protein finally resided in the plastid and stemming from an organism selected from the group consisting of the species:

Acetabularia mediterranea, Arabidopsis thaliana, Brassica campestris, Brassica napus, Capsicum annuum, Chlamydomonas reinhardtii, Cucurbita moschata, Dunaliella salina, Dunaliella tertiolecta, Euglena gracilis, Flaveria trinervia, Glycine max, Helianthus annuus, Hordeum vulgare, Lemna gibba, Lolium perenne, Lycopersion esculentum, Malus domestica, Medicago falcata, Medicago sativa, Mesembryanthemum crystallinum, Nicotiana plumbaginifolia, Nicotiana sylvestris, Nicotiana tabacum, Oenotherea hookeri, Oryza sativa, Petunia hybrida, Phaseolus vulgaris, Physcomitrella patens, Pinus tunbergii, Pisum sativum, Raphanus sativus, Silene pratensis, Sinapis alba, Solanum tuberosum, Spinacea oleracea, Stevia rebaudiana, Synechococcus, Synechocystis, Triticum aestivum and Zea mays.

Even more preferred nucleic acid sequences are encoding plastidal transit peptides as disclosed by von Heijne et al. (Plant Molecular Biology Reporter, 9 (2), 104 (1991)), which are hereby incorporated by reference. Table a shows some examples of the transit peptide sequences disclosed by von Heijne et al. According to the disclosure of the invention especially in the examples the skilled worker is able to link other nucleic acid sequences disclosed by von Heijne et al. to the respective nucleic acid sequences shown in Table I, columns 5 or 8, preferably the respective coding region thereof, or homologs or fragments thereof. Most preferred nucleic acid sequences encoding transit peptides are derived from the genus Spinacia such as chloroplast 30S ribosomal protein PSrp-1, root acyl carrier protein II, acyl carrier protein, ATP synthase: γ subunit, ATP synthase: δ subunit, cytochrom f, ferredoxin I, ferredoxin NADP oxidoreductase (=FNR), nitrite reductase, phosphoribulokinase, plastocyanin or carbonic anhydrase. The skilled worker will recognize that various other nucleic acid sequences encoding transit peptides can easily isolated from plastid-localized proteins, which are expressed from nuclear genes as precursors and are then targeted to plastids. Such transit peptides encoding sequences can be used for the construction of other expression constructs. The transit peptides advantageously used in the inventive process and which are part of the inventive nucleic acid sequences and proteins are typically 20 to 120 amino acids, preferably 25 to 110, 30 to 100 or 35 to 90 amino acids, more preferably 40 to 85 amino acids and most preferably 45 to 80 amino acids in length and function post-translationally to direct the protein to the plastid, preferably to the chloroplast. The nucleic acid sequences encoding such transit peptides are localized upstream of nucleic acid sequence encoding the mature protein. For the correct molecular joining of the transit peptide encoding nucleic acid and the nucleic acid encoding the protein to be targeted it is sometimes necessary to introduce additional base pairs at the joining position, which form restriction enzyme recognition sequences useful for the molecular joining of the different nucleic acid molecules. This procedure might lead to very few additional amino acids at the N-terminal of the mature imported protein, which usually and preferably do not interfere with the protein function. In any case, the additional base pairs at the joining position which form restriction enzyme recognition sequences have to be chosen with care, in order to avoid the formation of stop codons or codons which encode amino acids with a strong influence on protein folding, like e.g. proline. It is preferred that such additional codons encode small n.d. structural flexible amino acids such as glycine or alanine.

As mentioned above the nucleic acid sequences coding for the proteins as shown in Table II, application no. 8, column 5 or 8, or homologs or fragments thereof, are joined to a nucleic acid sequence encoding a plastidic transit peptide, e.g, if for the nucleic acid molecule in Table I, column 6 the term “plastidic” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in Table II, application no. 8, column 5 or 8, or homologs or fragments thereof, e.g., if for the nucleic acid molecule in Table I, column 6 the term “plastidic” is indicated.

Other plastid transit peptides are disclosed by Schmidt et al. (J. Biol. Chem. 268 (36), 27447 (1993)), Della-Cioppa et al. (Plant. Physiol. 84, 965 (1987)), de Castro Silva Filho et al. (Plant Mol. Biol. 30, 769 (1996)), Zhao et al. (J. Biol. Chem. 270 (11), 6081 (1995)), Römer et al. (Biochem. Biophys. Res. Commun., 196 (3), 1414 (1993)), Keegstra et al. (Annu. Rev. Plant Physiol. Plant Mol. Biol. 40, 471 (1989)), Lubben et al. (Photosynthesis Res. 17, 173 (1988)) and Lawrence et al. (J. Biol. Chem. 272, (33), 20357 (1997)), Cho et al. (Planta 224, 598 (2006)), Lucia et al. (Transgenic R. 17 (4), 529 (2008)), Murayama et al. (Planta 225 (5), 1193 (2007)). A general review about targeting is disclosed by Allison R. Kermode in Critical Reviews in Plant Science 15 (4), 285 (1996).

Favored plastidal transit peptide sequences, which are used in an embodiment of the inventive process and which form part of the inventive nucleic acid sequences are generally enriched in hydroxylated amino acid residues (serine and threonine), with these two residues generally constituting 20-35% of the total. They often have an amino-terminal region empty of Gly, Pro, and charged residues. Furthermore they have a number of small hydrophobic amino acids such as valine and alanine and generally acidic amino acids are lacking. In addition they generally have a middle region rich in Ser, Thr, Lys and Arg. Overall they have very often a net positive charge.

Advantageously mitochondrial transit peptides, which are beneficially used in an embodiment of the process of the present invention, are derived from the nucleic acid sequence encoding a protein selected from the group consisting of 22 kDA heat shock protein; 70 kDA heat shock protein; 83 kDA heat shock protein; 40S ribosomal protein S19; 50S ribosomal protein L15; ribosomal protein L29; 22 kDA PSST protein of complex I; 2-oxoacid dehydrogenase family protein; 2-oxoglutarate/malate translocator; 3methyl-2-oxobutanoate hydroxymethyltransferase; 3-Methylcrotonyl-coenzyme A carboxylase (MCCase); 7,8-Dihydropteroate synthase (DHPS)/6-hydroxymethyl-7,8-dihydropterine pyrophosphokinase (HPPK); aconitate hydratase; acyl carrier protein (ACP); ADP/ATP translocase; alanyl-tRNA synthetase; alcohol dehydrogenase (ADH); alternative oxidase (AOX); aminoacyl-tRNA ligase; asparate aminotransferase; ATP synthase alpha subunit; ATP synthase beta subunit; ATP synthase delta subunit; ATP synthase epsilon subunit; ATP synthase gamma subunit; ATP-dependent Clp protease-proteolytic subunit; Chaperonin 60-CPN60; Chaperonin 60 (2)-CPN60-2; Chaperonin 60(1)-CPN60-1; citrate synthase; cytochrome b-c1 complex subunit Rieske FeS Protein; cytochrome c reductase-processing peptidase subunit II; dihydrolipoamide S-acetyltransferase; farnesyl-diphosphate synthase 1; formate dehydrogenase; fumarate hydratase; gamma carbonic anhydrase protein (gammaCA); gamma carbonic anhydrase-like protein 1 (gammaCAL1); gamma carbonic anhydrase-like protein 2 (gammaCAL2); gamma-aminobutyric acid transaminase (GABA-T); glutathione reductase (GR); glycine decarboxylase subunit H; glycine decarboxylase subunit L; glycine decarboxylase subunit P; glycine decarboxylase subunit T; isovaleryl-CoA dehydrogenase (IVD); lipoamide dehydrogenase; malate oxidoreductase; manganese superoxide dismutase (Mn)SOD; methylmalonate-semialdehyde dehydrogenase; mitochondrial-processing peptidase beta subunit (MPP); mitochondrialprocessing peptidase subunit alpha (MPP); monodehydroascorbate reductase (MDHAR; NAD dependent isocitrate dehydrogenase; NAD dependent malate dehydrogenase; NAD-dependent malic enzyme; NAD-dependent malic enzyme 59 kDa isoform; NAD-dependent malic enzyme 62 kDa isoform; NAD-dependent malic enzyme 65 kDa isoform; NADH ubiquinone oxidoreductase 29 kDa subunit; NADH-ubiquinone oxidoreductase 18 kDa subunit; NADH-ubiquinone oxidoreductase 20 kDa subunit; NADH-ubiquinone oxidoreductase 23 kDa subunit; NADH-ubiquinone oxidoreductase 75 kDa subunit; NADP dependent isocitrate dehydrogenase; NADP dependent malate dehydrogenase; nucleoside diphosphate kinase; nucleoside diphosphate kinase III; o-acetylserine (thiol) lyase (OAS-TL) ; propionyl-CoA carboxylase; protoporphyrinogen IX oxidase; pyruvate dehydrogenase E1 component subunit alpha; serine acetyltransferase (SAT); serine hydroxymethyltransferase; succinate dehydrogenase (SDH); succinic semialdehyde dehydrogenase (SSADH); succinyl-CoA ligase (GDP-forming) alpha-chain; succinyl-CoA ligase [GDP-forming] subunit beta; thiosulfate sulfurtransferase; threonyl-tRNA synthetase; trans-2-enoyl-CoA reductase; translocase inner membrane (TIM); translocase outer membrane (TOM); tRNA synthetase class I and ubiquinol cytochrome C oxidoreductase complex.

More preferred the nucleic acid sequence encoding a mitochondrial transit peptide is derived from a nucleic acid sequence encoding a protein finally resided in the mitochondrion and stemming from an organism selected from the group consisting of the species:

Acetabularia mediterranea, Arabidopsis thaliana, Brassica campestris, Brassica napus, Capsicum annuum, Chlamydomonas reinhardtii, CururbitaCucurbita moschata, Daucus carota, Dunaliella salina, Dunaliella tertiolecta, Euglena gracilis, Flaveria trinervia, Glycine max, Helianthus annuus, Hordeum vulgare, Lactuca sativa, Lemna gibba, Lolium perenne, Lycopersion esculentum, Malus domestica, Medicago falcata, Medicago sativa, Mesembryanthemum crystallinum, Nicotiana plumbaginifolia, Nicotiana sylvestris, Nicotiana tabacum, Oenotherea hookeri, Oryza sativa, Petunia hybrida, Phaseolus vulgaris, Physcomitrella patens, Pinus tunbergii, Pisum sativum, Pyrus pyrifolia, Raphanus sativus, Saccharum officinarum, Silene pratensis, Sinapis alba, Solanum tuberosum, Spinacea oleracea, Stevia rebaudiana, Synechococcus, Synechocystis, Triticum aestivum and Zea mays.

Even more preferred nucleic acid sequences are encoding mitochondrial transit peptides as disclosed by White and Scandalios (Proc. Natl. Acad. Sci., 86 (10), 3534 (1989)), Cho et al (Plant Physiol. 149, 745 (2009)), Chatre et al (Journal of Experimental Botany, 60 (3), 741 (2009)), Murayama et al (Planta 225 (5), 1193 (2007) and Manzano et al (2006) , which are hereby incorporated by reference. Table b shows some examples of the transit peptide sequences disclosed by White and Scandalios (1989), Cho et al (2009), Chatre et al (2009), Murayama et al (2007) and Manzano et al (2006), Glaser et al (Plant Mol Biol. 38 (1-2), 311 (1998) and Huang et al (Plant Phys. 150, 1272 (2009)). According to the disclosure of the invention especially in the examples the skilled worker is able to link other nucleic acid sequences disclosed by Glaser et al (Plant Mol Biol. 38 (1-2), 311 (1998)) and Huang et al (Plant Phys. 150, 1272 (2009)) to the respective nucleic acid sequences shown in Table I, columns 5 or 8, preferably the coding region thereof, or a homolog or a fragment thereof. Most preferred nucleic acid sequences encoding transit peptides are derived from the genus Arabidopsis thaliana such as mitochondrial malate oxidoreductase, manganese superoxide dismutase (Mn)SOD, translocase inner membrane (TIM), translocase outer membrane (TOM), ATP synthase alpha subunit, ATP synthase beta subunit, ATP synthase gamma subunit, ATP synthase delta subunit, ATP synthase epsilon subunit, ADP/ATP translocase, aconitate hydratase and isovaleryl-CoA dehydrogenase (IVD). The skilled worker will recognize that various other nucleic acid sequences encoding transit peptides can easily isolated from mitochondrion-localized proteins, which are expressed from nuclear genes as precursors and are then targeted to mitochondria. Such transit peptides encoding sequences can be used for the construction of other expression constructs. The transit peptides advantageously used in the inventive process and which are part of the inventive nucleic acid sequences and proteins are typically 20 to 120 amino acids, preferably 25 to 110, 30 to 100 or 35 to 90 amino acids, more preferably 40 to 85 amino acids and most preferably 45 to 80 amino acids in length and function post-translationally to direct the protein to the mitochondrion. The nucleic acid sequences encoding such transit peptides are localized upstream of nucleic acid sequence encoding the mature protein. For the correct molecular joining of the transit peptide encoding nucleic acid and the nucleic acid encoding the protein to be targeted it is sometimes necessary to introduce additional base pairs at the joining position, which forms restriction enzyme recognition sequences useful for the molecular joining of the different nucleic acid molecules. This procedure might lead to very few additional amino acids at the N-terminal of the mature imported protein, which usually and preferably do not interfere with the protein function. In any case, the additional base pairs at the joining position which forms restriction enzyme recognition sequences have to be chosen with care, in order to avoid the formation of stop codons or codons which encode amino acids with a strong influence on protein folding, like e.g. proline. It is preferred that such additional codons encode small n.d. structural flexible amino acids such as glycine or alanine.

As mentioned above the nucleic acid sequences coding for the proteins as shown in the respective line in Table II, application no. 8, column 5 or 8, or homologs or fragments thereof, are joined to a nucleic acid sequence encoding a mitochondric transit peptide, e.g., if for the respective nucleic acid molecule in Table I, column 6 the term “mitochondrial” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in the respective line in Table II, application no. 8, column 5 or 8, or homologs or fragments thereof, e.g., if for the nucleic acid molecule in the respective line in Table I, column 6 the term “mitochondrial” is indicated. (For the avoidance of doubt, the term “mitochondric” and “mitochondrial” are interchangeable.)

Other mitochondric transit peptides are disclosed by Cho et al. (Plant Physiol. 149, 745 (2009)), Gnanasambandam et al. (Functional Plant Biology 35(2), 166, (2008)), Subbaiah et al. (J. Biol.Chem. 281 (23), 15625 (2006)), Murcha et al. (Plant Physiol. 138, 2134 (2005)), Chatre et al. (Journal of Experimental Botany, 60 (3), 741 (2009)), Millar and Heazlewood (Plant Physiol. 131, 443 (2003)), White and Scandalios (Proc. Natl. Acad. Sci., 86 (10), 3534 (1989)), Laloi et al. (Proc. Natl. Acad. Sci., 98 (24), 14144 (2001)), Barranco-Medina et al. (Plant Physiol. and Biochem. 45, 729 (2007)), Olejnik et al. (FEBS J. 274 (18), 4877 (2007)), Fabre et al. (Plant Cell and Env. 30 (5), 617 (2007)), Murayama et al. (Planta 225 (5), 1193 (2007)), Bathgate et al. (Eur. J. Biochem. 183, 303 (1989)), Winning et al. (Plant J. 2, 763 (1992)), Murcha et al. (Plant Physiol. 143, 199 (2007)), Lehnerer et al. (Botanica Acta, 107, 306 (1994)), Kimura et al. (J. Biol. Chem. 265 (11), 6079 (1990)), Yang et al. (Plant Mol. Biol., 62, 951 (2006)).

Favored mitochondric transit peptide sequences, which are used in an embodiment of the inventive process and which form part of the inventive nucleic acid sequences are generally enriched in hydroxylated amino acid residues (serine and threonine), with these two residues generally constituting 20-35% of the total. They often have an amino-terminal region empty of Gly, Pro, and charged residues. Furthermore they have a number of small hydrophobic amino acids such as valine and alanine and generally acidic amino acids are lacking.

In addition they generally have a middle region rich in Ser, Thr, Lys and Arg. Overall they have very often a net positive charge.

Nucleic acid sequences coding for the transit peptides may be chemically synthesized either in part or wholly according to structure of transit peptide sequences disclosed in the prior art. Said natural or chemically synthesized sequences can be directly linked to the sequences encoding the mature protein or via a linker nucleic acid sequence, which may be typically less than 500 base pairs, preferably less than 450, 400, 350, 300, 250 or 200 base pairs, more preferably less than 150, 100, 90, 80, 70, 60, 50, 40 or 30 base pairs and most preferably less than 25, 20, 15, 12, 9, 6 or 3 base pairs in length and are in frame to the coding sequence. Furthermore favorable nucleic acid sequences encoding transit peptides may comprise sequences derived from more than one biological and/or chemical source and may include a nucleic acid sequence derived from the amino-terminal region of the mature protein, which in its native state is linked to the transit peptide. In a preferred embodiment of the invention said amino-terminal region of the mature protein is typically less than 150 amino acids, preferably less than 140, 130, 120, 110, 100 or 90 amino acids, more preferably less than 80, 70, 60, 50, 40, 35, 30, 25 or 20 amino acids and most preferably less than 19, 18, 17, 16, 15, 14, 13, 12, 11 or 10 amino acids in length. But even shorter or longer stretches are also possible. In addition target sequences, which facilitate the transport of proteins to other cell compartments such as the vacuole, endoplasmic reticulum, golgi complex, glyoxysomes, peroxisomes, mitochondria (in case of a plastidal transit peptide) or plastids, especially chloroplasts (in case of a mitochondric transit peptide) may be also part of the inventive nucleic acid sequence. The proteins translated from said inventive nucleic acid sequences are a kind of fusion proteins that means the nucleic acid sequences encoding the plastidal transit peptide for example the ones shown in Table a, preferably the last one of the Table a are joint to the respective nucleic acid sequences shown in Table I, application no. 8, columns 5 or 8, especially the coding region thereof, or homologs or fragments thereof, in case of nucleic acid sequences being expressed in plastids, and that means the nucleic acid sequences encoding the mitochondric transit peptide for example the ones shown in Table b, preferably the last one of the Table b are joint to the respective nucleic acid sequences shown in Table I, application no. 8, columns 5 or 8, especially the coding region thereof, or homologs or fragments thereof, in case of nucleic acid sequences being expressed in mitochondria. The person skilled in the art is able to join said sequences in a functional manner. Advantageously the transit peptide part is cleaved off from the protein part shown in Table II, application no. 8, columns 5 or 8, or a homolog or fragment thereof, during or after the transport into the organelles, preferably into the plastids or mitochondria, respectively.

All products of the cleavage of the preferred transit peptide shown in the last line of Table a have preferably the N-terminal amino acid sequences QIA CSS or QIA EFQLTT in front of the start methionine of the respective protein mentioned in Table II, application no. 8, column 5 or 8. or homologs or fragments thereof. Other short amino acid sequences of an range of 1 to 20 amino acids preferable 2 to 15 amino acids, more preferable 3 to 10 amino acids most preferably 4 to 8 amino acids are also possible in front of the start methionine of the protein mentioned in Table II, application no. 8, columns 5 or 8, or homologs or fragments thereof. In case of the amino acid sequence QIA CSS the three amino acids in front of the start methionine are stemming from the LIC (=ligatation independent cloning) cassette. Said short amino acid sequence is preferred in the case of the expression of E. coli genes. In case of the amino acid sequence QIA EFQLTT the six amino acids in front of the start methionine are stemming from the LIC cassette. Said short amino acid sequence is preferred in the case of the expression of S. cerevisiae genes. The skilled worker knowns that other short sequences are also useful in the expression of the respective nucleic acid molecule comprising a polynucleotide as depicted in Table I, application no. 8, column 5 or 8, preferably the coding region thereof, or a homolog or a fragment thereof. Furthermore the skilled worker is aware of the fact that there is not a need for such short sequences in the expression of the genes.

TABLE A
Examples of plastidal transit peptides disclosed by von Heijne et al.

| Trans | | | SEQ | |
|---|---|---|---|---|
| Pep | Organism | Transit Peptide | ID NO. | Reference |
| 1 | Acetabularia | MASIMMNKSVVLSKECAKPLATPK | 1 | Mol. Gen. |
| | mediterranea | VTLNKRGFATTIATKNREMMVWQP | | Genet. 218, |
| | | FNNKMFETFSFLPP | | 445(1989) |
| 2 | Arabidopsis | MAASLQSTATFLQSAKIATAPSRG | 2 | EMBO J. 8, |
| | thaliana | SSHLRSTQAVGKSFGLETSSARLT | | 3187 |
| | | CSFQSDFKDFTGKCSDAVKIAGFA | | (1989) |
| | | LATSALVVSGASAEGAPK | | |
| 3 | Arabidopsis | MAQVSRICNGVQNPSLICNLSKSS | 3 | Mol. Gen. |
| | thaliana | QRKSPLSVSLKTQQHPRAYPISSS | | Genet. 210, |
| | | WGLKKSGMTLIGSELRPLKVMSSV | | 437 (1987) |
| | | STAEKASEIVLQPIREISGLIKLP | | |
| 4 | Arabidopsis | MAAATTTTTTSSSISFSTKPSPSS | 4 | Plant |
| | thaliana | SKSPLPISRFSLPFSLNPNKSSSS | | Physiol. 85, |
| | | SRRRGIKSSSP SS ISAVLNTTTNV | | 1110 |
| | | TTTPSPTKPTKPETF ISRFAPDQP | | (1987) |
| | | RKGA | | |
| 5 | Arabidopsis | MITSSLTCSLQALKLSSPFAHGST | 5 | J. Biol. |
| | thaliana | PLSSLSKPNSFPNHRMPALVPV | | Chem. 265, |
| | | | | 2763 |
| | | | | (1990) |
| 6 | Arabidopsis | MASLLGTSSSAIWASPSLSSPSSK | 6 | EMBO J. |
| | thaliana | PSSSPICFRPGKLFGSKLNAGIQI | | 9,1337 |
| | | RPKKNRSRYHVSVMNVATEINSTE | | (1990) |
| | | QVVGKFDSKKSARPVYPFAAI | | |
| 7 | Arabidopsis | MASTALSSAIVGTSFIRRSPAPISL | 7 | Plant |
| | thaliana | RSLPSANTQSLFGLKSGTARGG | | Physiol. 93, |
| | | RVVAM | | 572 (1990) |
| 8 | Arabidopsis | MAASTMALSSPAFAGKAVNLSPAA | 8 | Nucl. Acids |
| | thaliana | SEVLGSGRVTNRKTV | | Res. 14, |
| | | | | 4051 |
| | | | | (1986) |
| 9 | Arabidopsis | MAAITSATVTIPSFTGLKLAVSSK | 9 | Gene 65, |
| | thaliana | PKTLSTISRSSSATRAPPKLALKS | | 59 (1988) |
| | | SLKDFGVIAVATAASIVLAGNAMA | | |
| | | MEVLLGSDDGSLAFVPSEFT | | |
| 10 | Arabidopsis | MAAAVSTVGAINRAPLSLNGSGSG | 10 | Nucl. Acids |
| | thaliana | AVSAPASTFLGKKVVTVSRFAQSN | | Res. 17, |
| | | KKSNGSFKVLAVKEDKQTDGDRWR | | 2871 |
| | | GLAYDTSDDQIDI | | (1989) |
| 11 | Arabidopsis | MkSSMLSSTAWTSPAQATMVAPF | 11 | Plant Mol. |
| | thaliana | TGLKSSASFPVTRKANNDITSITS | | Biol. 11, |
| | | NGGRVSC | | 745 (1988) |
| 12 | Arabidopsis | MAASGTSATFRASVSSAPSSSSQL | 12 | Proc. Natl. |
| | thaliana | THLKSPFKAVKY TPLPS SRSKSSS | | Acad. Sci. |
| | | FSVSCTIAKDPPVLMAAGSDPALW | | USA 86, |
| | | QRPDSFGRFGKFGGKYVPE | | 4604 |
| | | | | (1989) |
| 13 | Brassica | MSTTFCSSVCMQATSLAATTRISF | 13 | Nucl. Acids |
| | campestris | QKPALVSTTNLSFNLRRSIPTRFS | | Res. 15, |
| | | ISCAAKPETVEKVSKIVKKQLSLK | | 7197 |
| | | DDQKVVAE | | (1987) |
| 14 | Brassica | MATTFSASVSMQATSLATTTRISF | 14 | Eur. J. Bio- |
| | napus | QKPVLVSNHGRTNLSFNLSRTRLSISC | | chem. 174, |
| | | | | 287 (1988) |
| 15 | Chlamydomonas | MQALSSRVNIAAKPQRAQRLVVRA | 15 | Plant Mol. |
| | reinhardtii | EEVKAAPKKEVGPKRGSLVK | | Biol. 12, |
| | | | | 463 (1989) |
| 16 | Cucurbita | MAELIQDKESAQSAATAAAASSGY | 16 | FEBS Lett. |
| | moschata | ERRNEPAHSRKFLEVRSEEELLSCIKK | | 238, 424 |
| | | | | (1988) |
| 17 | Spinacea | MSTINGCLTSISPSRTQLKNTSTL | 17 | J. Biol. |
| | oleracea | RPTFIANSRVNPSSSVPPSLIRNQ | | Chem. 265, |
| | | PVFAAPAPIITPTL | | 5414 |
| | | | | (1990) |
| 18 | Spinacea | MTTAVTAAVSFPSTKTTSLSARCS | 18 | Curr. |
| | oleracea | SVISPDKISYKKVPLYYRNVSATG | | Genet. 13, |
| | | KMGPIRAQIASDVEAPPPAPAKVEKMS | | 517 (1988) |
| 19 | Spinacea | MTTAVTAAVSFPSTKTTSLSARSS | 19 | |
| | oleracea | SVISPDKISYKKVPLYYRNVSATG | | |
| | | KMGPIRA | | |

TABLE b
Examples of mitochondric transit peptides disclosed by White and
Scandalios (1989); Cho et al (2009); Chatre et al (2008);
Murayama et al (2007); Manzano et al (2006):

| Trans | | | SEQ | |
|---|---|---|---|---|
| Pep | Organism | Transit Peptide | ID NO. | Reference |
| 20 | Zea mays | MALRTLASKKVLSFPFGGAGRPLAAAAS | 63 | Proc. Nat. |
| | | ARGV | | Acad.Sci. |
| | | | | 86, 3534 |
| | | | | (1989) |
| 21 | Oryza sativa | MGKAAAVGTAVVVAAAVGVAVVLA | 64 | Plant |
| | | | | Physiol |
| | | | | 149, 745 |
| | | | | (2009) |
| 22 | Oryza sativa | MGKGTVVGTAVVVCAAAAAAVGVAVVVS | 65 | Plant |
| | | | | Physiol |
| | | | | 149, 745 |
| | | | | (2009) |
| 23 | Arabidosis | MIITTHKRDINLLVLQLGAALAVSFAGF | 66 | J. Exp. Bot. |
| | thaliana | LFARFRKNTKR | | 60 (3), 741 |
| | | | | (2009) |
| 24 | Oryza sativa | MAAAAISHLRRGAPRHARALYLSTR- | 67 | Planta 225 |
| | | RFSSSSAAGVAPLAAVAASARR- | | (5), 1193 |
| | | LLSTSVDSGASSGESYKPPLFDPF- | | (2007) |
| | | RAASLASSAPPLESPPIEELPDDA- | | |
| | | TPPPEEEPGLPAPEKDPVATACQHE- | | |
| | | LEGLKAVETVRSRKESTEEKEAWSL- | | |
| | | LGRSVVSYCGTA | | |
| 25 | Arabidosis | MSVSCCCRNLGKTIKKAIPSHHLHL- | 68 | Plant Mol. |
| | thaliana | RSLGGSLYRRRIQSSSMETDLKS | | Bio. 61, |
| | | | | 195 (2006) |

Alternatively to the targeting of the respective sequences shown in Table II, application no. 8, columns 5 or 8, or homologs or fragments thereof, preferably of sequences in general encoded in the nucleus with the aid of the targeting sequences mentioned for example in Table a alone, Table b alone, or in combination with other targeting sequences preferably into plastids or mitochondria, respectively, the nucleic acids of the invention can directly be introduced into the plastidal or mitochondrial genome. Therefore in a preferred embodiment the respective nucleic acid sequences shown in the respective line in Table I, application no. 8, columns 5 or 8, preferably the coding region thereof, or homologs or fragments thereof, are directly introduced and expressed in plastids e.g., if for the nucleic acid molecule in the respective line in Table I, column 6, the term “plastidic” is indicated, or are directly introduced and expressed in mitochondria e.g., if for the nucleic acid molecule in the respective line in Table I, column 6 the term “mitochondrial” is indicated.

The term “introduced” in the context of this specification shall mean the insertion of a nucleic acid sequence into the organism by means of a “transfection”, “transduction” or preferably by “transformation”.

A plastid, such as a chloroplast, or a mitochondrion, has been “transformed” by an exogenous (preferably foreign) nucleic acid sequence if a nucleic acid sequence has been introduced into the plastid or mitochondrion that means that this sequence has crossed the membrane or the membranes of the plastid or of the mitochondrion. The exogenous, preferably foreign, DNA may be integrated (covalently linked) into plastid or mitochondrial DNA making up the genome of the plastid or mitochondrion, or it may remain unintegrated (e.g., by including a chloroplast origin of replication). “Stably” integrated DNA sequences are those, which are inherited through plastid or mitochondrion replication, thereby transferring new plastids or mitochondria, with the features of the integrated DNA sequence to the progeny.

For expression a person skilled in the art is familiar with different methods to introduce the nucleic acid sequences into different organelles, such as the preferred plastids or mitochondria. Such methods are for example disclosed by Pal Maiga (Annu. Rev. Plant Biol. 55, 289, (2004)), Evans T. (WO 2004/040973), McBride K.E. et al. (U.S. Pat. No. 5,455,818), Daniell H. et al. (U.S. Pat. No. 5,932,479 and U.S. Pat. No. 5,693,507) and Straub J. M. et al. (US 6,781,033). A preferred method is the transformation of microspore-derived hypocotyl or cotyledonary tissue (which are green and thus contain numerous plastids) leaf tissue and afterwards the regeneration of shoots from said transformed plant material on selective medium. As methods for the transformation bombarding of the plant material or the use of independently replicating shuttle vectors are well known by the skilled worker. But also a PEG-mediated transformation of plastids or Agrobacterium transformation with binary vectors are possible. Useful markers for the transformation of plastids are positive selection markers for example the chloramphenicol-, streptomycin-, kanamycin-, neomycin-, amikamycin-, spectinomycin-, triazine- and/or lincomycin-resistance genes. As additional markers named in the literature often as secondary markers, genes coding for the resistance against herbicides such as phosphinothricin (=glufosinate, BASTA™, Liberty™, encoded by the bar gene), glyphosate (=N-(phosphonomethyl)glycine, Roundup Ready™, encoded by the 5-enolpyruvylshikimaete-3-phosphate synthase gene=epsps), sulfonylurea (e.g. =Staple™, encoded by the acetolactate synthase gene), imidazolinone (=IMI, e.g. imazethapyr, imazamox, Clearfield™, encoded by the acetohydroxyacid synthase (AHAS) gene, also known as acetolactate synthase (ALS) gene) or bromoxynil (=Buctril™, encoded by the oxy gene) or genes coding for antibiotics such as hygromycin or G418 are useful for further selection. Such secondary markers are useful in the case when most genome copies are transformed. In addition negative selection markers such as the bacterial cytosine deaminase (encoded by the codA gene) are also useful for the transformation of organelles, especially plastids.

Thus, in one embodiment, an activity disclosed herein as being conferred by a polypeptide shown in the respective line in Table II, or a homolog or a fragment thereof, is increased or generated by linking the polypeptide disclosed in respective line in Table II, or a homolog or a fragment thereof, or a polypeptide conferring the same said activity with a targeting signal as herein described, if in the respective line in column 6 of Table II the term “plastidic” is listed for said polypeptide. For example, the polypeptide described can be linked to the targeting signal shown in Table a.

Accordingly, in the method of the invention for producing a transgenic plant with increased fine chemical production as compared to a corresponding, e.g. non-transformed, wild type plant, comprising transforming a non-human organism, preferably a plant cell, a plant or a part thereof, with the mentioned nucleic acid molecule, said nucleic acid molecule selected from said mentioned group encodes for a polypeptide conferring said activity being linked to a targeting signal as mentioned herein, e.g. as mentioned in Table a, e.g. if in the respective line in column 6 of Table II the term “plastidic” is listed for the encoded polypeptide.

To increase the possibility of identification of transformants it is possible to use reporter genes other then the aforementioned resistance genes or in addition to said genes. Reporter genes are for example β-galactosidase-, β-glucuronidase-(GUS), alkaline phosphatase- and/or green-fluorescent protein-genes (GFP).

For the process of the present invention it may be of great advantage that by transforming the plastids the intraspecies specific transgene flow is blocked, because a lot of species such as corn, cotton and rice have a strict maternal inheritance of plastids. By placing the respective genes specified in the respective line in Table I, application no. 8, columns 5 or 8, preferably the coding region, or homologs or fragments thereof, e.g. if for the nucleic acid molecule in the respective line in column 6 of Table I the term “plastidic” is indicated, in the plastids of plants, these genes will not be present in the pollen of said plants.

A further preferred embodiment of the invention relates to the use of so called “chloroplast localization sequences”, in which a first RNA sequence or molecule is capable of transporting or “chaperoning” a second RNA sequence, such as a RNA sequence transcribed from the sequences depicted in the respective line in Table I, application no. 8, columns 5 or 8, preferably the coding region thereof, or a homolog or a fragment thereof, or a sequence encoding a protein, as depicted in the respective line in Table II, application no. 8, columns 5 and 8, or a homolog or a fragment thereof, from an external environment inside a cell or outside a plastid into a chloroplast. In one embodiment the chloroplast localization signal is substantially similar or complementary to a complete or intact viroid sequence. The chloroplast localization signal may be encoded by a DNA sequence, which is transcribed into the chloroplast localization RNA. The term “viroid” refers to a naturally occurring single stranded RNA molecule (Flores, C. R. Acad. Sci. III. 324 (10), 943 (2001)). Viroids usually contain about 200-500 nucleotides and generally exist as circular molecules. Examples of viroids that contain chloroplast localization signals include but are not limited to ASBVd, PLMVd, CChMVd and ELVd. The viroid sequence or a functional part of it can be fused to the sequences depicted in the respective line in Table I, application no. 8, columns 5 or 8, preferably the coding region thereof, or a homolog or a fragment thereof, or a sequence encoding a protein, as depicted in the respective line in Table II, application no. 8, columns 5 or 8, or a homolog or a fragment thereof, in such a manner that the viroid sequence transports a sequence transcribed from a sequence as depicted in the respective line in Table I, application no. 8 columns 5 or 8, preferably the coding region thereof, or a homolog or a fragment thereof, or a sequence encoding a protein as depicted in the respective line in Table II, application no. 8 columns 5 or 8, or a homolog or a fragment thereof, into the chloroplasts, e.g. if for the nucleic acid molecule in column 6 of Table I the term “plastidic” is indicated. A preferred embodiment uses a modified ASBVd (Navarro et al., Virology. 268 (1), 218 (2000)).

In a further specific embodiment the protein to be expressed in the plastids such as the proteins depicted in the respective line in Table II, application no. 8, columns 5 or 8, or a homolog or a fragment thereof, e.g. if for the nucleic acid molecule in the respective line in column 6 of Table I the term “plastidic” is indicated, are encoded by different nucleic acids. Such a method is disclosed in WO 2004/040973, which shall be incorporated by reference. WO 2004/040973 teaches a method, which relates to the translocation of an RNA corresponding to a gene or gene fragment into the chloroplast by means of a chloroplast localization sequence.

The genes, which should be expressed in the plant or plant cells, are split into nucleic acid fragments, which are introduced into different compartments in the plant e.g. the nucleus, the plastids and/or mitochondria. Additionally plant cells are described in which the chloroplast contains a ribozyme fused at one end to an RNA encoding a fragment of a protein used in the inventive process such that the ribozyme can trans-splice the translocated fusion RNA to the RNA encoding the gene fragment to form and as the case may be reunite the nucleic acid fragments to an intact mRNA encoding a functional protein for example as disclosed in the respective line in Table II, application no. 8, columns 5 or 8, or a homolog or a fragment thereof.

In a preferred embodiment of the invention the nucleic acid sequences as shown in the respective line in Table I, application no. 8, columns 5 or 8, preferably the coding region thereof, or homologs or fragments thereof, e.g. if for the nucleic acid molecule in the respective line in column 6 of Table I the term “plastidic” is indicated, used in the inventive process are transformed into plastids, which are metabolically active. Those plastids should preferably maintain at a high copy number in the plant or plant tissue of interest, most preferably the chloroplasts found in green plant tissues, such as leaves or cotyledons or in seeds.

For a good expression in the plastids the nucleic acid sequences as shown in the respective line in Table I, application no. 8, columns 5 or 8, preferably the coding region thereof, or homologs or fragments thereof, e.g. if for the nucleic acid molecule in the respective line in column 6 of Table I the term “plastidic” is indicated, are introduced into an expression cassette using preferably a promoter and terminater, which are active in plastids preferably a chloroplast promoter. Examples of such promoters include the psbA promoter from the gene from spinach or pea, the rbcL promoter, and the atpB promoter from corn.

In another embodiment, an activity disclosed herein as being conferred by a polypeptide shown in the respective line in Table II is increased or generated by linking the polypeptide disclosed in the respective line in Table II or a polypeptide conferring the same said activity with an targeting signal as herein described, if in the respective line in column 6 of Table II the term “mitochondrial” is listed for said polypeptide. For example, the polypeptide described can be linked to the targeting signal shown in Table b.

In these cases the respective organelle is a mitochondrion. However, the remarks made regarding the plastids are applicable in analogy.

In another embodiment, an activity disclosed herein as being conferred by a polypeptide shown in Table II is increased or generated by the polypeptide disclosed in the respective line in Table II or a polypeptide conferring the same said activity without linking said polypeptide shown in the respective line in Table II to a targeting signal, if in the respective line in column 6 of Table II the term “non-targeted” is listed for said polypeptide.

For the purposes of the description of the present invention, the terms “non-targeted” shall indicate, that the nucleic acid of the invention is expressed without the addition of a “non-natural transit peptide encoding sequence”. A “non-natural transit peptide encoding sequence” is a sequence which is not a natural part of a nucleic acid of the invention, e.g. of the nucleic acids depicted in the respective line in Table I column 5 or 8, preferably the coding region thereof, or homologs or fragments thereof, but which is rather added by molecular manipulation steps as for example described in the example under “plastid targeted expression”. However, the terms “non-targeted” shall not exclude a targeted localization to any cell compartment for the products of the inventive nucleic acid sequences by their naturally occurring sequence properties within the background of the transgenic organism. The subcellular location of the mature polypeptide derived from the enclosed sequences can be predicted by a skilled person for the organism (plant) by using software tools like TargetP (Emanuelsson et al., J. Mol. Biol. 300, 1005 (2000)), ChloroP (Emanuelsson et al., Protein Science 8, 978 (1999)) or other predictive software tools (Emanuelsson et al., Nature Protocols 2, 953 (2007)).

Preferably, the process according to the invention further comprises the step of recovering the fine chemical, which is synthesized by the organism from the organism and/or from the culture medium used for the growth or maintenance of the organism. The term “recovering” means the isolation of the fine chemical in different purities, that means on the one hand harvesting of the biological material, which contains the fine chemical without further purification and on the other hand purities of the fine chemical between 5% and 100% purity, preferred purities are in the range of 10% and 99%. In one embodiment, the purities are at least 20%, 30%, 40%, 50%, 60%, 70%, 80%, 90%, 95% or 99%.

Advantageously the process for the production of the fine chemical linoleic acid leads to an enhanced production of the respective fine chemical. The terms “enhanced” or “increase” mean at least a 10%, 20%, 30%, 40% or 50%, preferably at least 60%, 70%, 80%, 90% or 100%, more preferably 150%, 200%, 300%, 400% or 500% higher production of the respective fine chemical linoleic acid in comparison to the wild-type as defined above, e.g. that means in comparison to a non-human organism without the aforementioned modification of the activity of a protein as shown in the respective line in Table II, application no. 8, column 5 or 8, or a fragment or a homolog thereof. The modification of the activity of a protein as shown in the respective line in Table II, application no. 8, column 5 or 8, or a homolog or a fragment thereof, or their combination can be achieved by joining the protein to a respective transit peptide, e.g. if for the respective encoding nucleic acid molecule in column 6 of Table I the term “plastidic” or “mitochondrial” is indicated.

Surprisingly it was found, that the transgenic expression of a protein as shown in the respective line in Table II, application no. 8, column 5, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, conferred a production of or an increase in linoleic acid,respectively, to the transgenic non-human organism as compared to a corresponding non-transformed wild type.

In one embodiment the transgenic expression of a protein as shown in the respective line in Table II, application no. 8, column 5 or 8, or a homolog or a fragment thereof, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, confers a production of or an increase in linoleic acid,respectively, to the transgenic non-human organism as compared to a corresponding non-transformed wild type.

Accordingly, in one embodiment, an increase of linoleic acid in a non-human organism, as compared to a corresponding non-transformed wild type non-human organism, is conferred in the process of the invention, if the activity of a polypeptide showing the activity of a chlorophyllase, or if the activity of the polypeptide At1g19670, preferably represented by SEQ ID NO. 113652, or a homolog or fragment thereof, or if the activity of a polypeptide encoded by a nucleic acid molecule comprising the nucleic acid SEQ ID NO. 113651, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 113651 or polypeptide SEQ ID NO. 113652, respectively, or a homolog or a fragment thereof, is increased or generated, or if the activity chlorophyllase is increased or generated in a non-human organism, like a microorganism or a plant cell, plant or part thereof, especially with non-targeted localization. For example, an increase of the linoleic acid of at least 1 percent, particularly in a range of 15 to 24-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a At1g19800-protein, or if the activity of the polypeptide At1g19800, preferably represented by SEQ ID NO. 17638, or a homolog or fragment thereof, or if the activity of a polypeptide encoded by a nucleic acid molecule comprising the nucleic acid SEQ ID NO. 17637, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 17637 or polypeptide SEQ ID NO. 17638, respectively, or a homolog or a fragment thereof, is increased or generated, or if the activity At1g19800-protein is increased or generated in a non-human organism, like a microorganism or a plant cell, plant or part thereof, especially with non-targeted localization. For example, an increase of the linoleic acid of at least 1 percent, particularly in a range of 14 to 29-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a transcription factor, or if the activity of the polypeptide At1g68320, preferably represented by SEQ ID NO. 1062, or a homolog or fragment thereof, or if the activity of a polypeptide encoded by a nucleic acid molecule comprising the nucleic acid SEQ ID NO. 1061, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 1061 or polypeptide SEQ ID NO. 1062, respectively, or a homolog or a fragment thereof, is increased or generated, or if the activity transcription factor is increased or generated in a non-human organism, like a microorganism or a plant cell, plant or part thereof, especially with non-targeted localization. For example, an increase of the linoleic acid of at least 1 percent, particularly in a range of 15 to 79-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a pyruvate kinase, or if the activity of the polypeptide At2g36580, preferably represented by SEQ ID NO. 113715, or a homolog or fragment thereof, or if the activity of a polypeptide encoded by a nucleic acid molecule comprising the nucleic acid SEQ ID NO. 113714, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 113714 or polypeptide SEQ ID NO. 113715, respectively, or a homolog or a fragment thereof, is increased or generated, or if the activity pyruvate kinase is increased or generated in a non-human organism, like a microorganism or a plant cell, plant or part thereof, especially with plastidic localization. For example, an increase of the linoleic acid of at least 1 percent, particularly in a range of 14 to 19-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a At2g45420-protein, or if the activity of the polypeptide At2g45420, preferably represented by SEQ ID NO. 1624, or a homolog or fragment thereof, or if the activity of a polypeptide encoded by a nucleic acid molecule comprising the nucleic acid SEQ ID NO. 1623, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 1623 or polypeptide SEQ ID NO. 1624, respectively, or a homolog or a fragment thereof, is increased or generated, or if the activity At2g45420-protein is increased or generated in a non-human organism, like a microorganism or a plant cell, plant or part thereof, especially with non-targeted localization. For example, an increase of the linoleic acid of at least 1 percent, particularly in a range of 16 to 42-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a heat shock transcription factor, or if the activity of the polypeptide At3g02990, preferably represented by SEQ ID NO. 68658, or a homolog or fragment thereof, or if the activity of a polypeptide encoded by a nucleic acid molecule comprising the nucleic acid SEQ ID NO. 68657, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 68657 or polypeptide SEQ ID NO. 68658, respectively, or a homolog or a fragment thereof, is increased or generated, or if the activity heat shock transcription factor is increased or generated in a non-human organism, like a microorganism or a plant cell, plant or part thereof, especially with non-targeted localization. For example, an increase of the linoleic acid of at least 1 percent, particularly in a range of 19 to 30-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a CCAAT-binding transcription factor, or if the activity of the polypeptide At3g20910, preferably represented by SEQ ID NO. 22922, or a homolog or fragment thereof, or if the activity of a polypeptide encoded by a nucleic acid molecule comprising the nucleic acid SEQ ID NO. 22921, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 22921 or polypeptide SEQ ID NO. 22922, respectively, or a homolog or a fragment thereof, is increased or generated, or if the activity CCAAT-binding transcription factor is increased or generated in a non-human organism, like a microorganism or a plant cell, plant or part thereof, especially with non-targeted localization. For example, an increase of the linoleic acid of at least 1 percent, particularly in a range of 14 to 29-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization. For example, an increase of the linoleic acid of at least 1 percent, particularly in a range of 16 to 30-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a transcription factor, or if the activity of the polypeptide At4g09960, preferably represented by SEQ ID NO. 109718, or a homolog or fragment thereof, or if the activity of a polypeptide encoded by a nucleic acid molecule comprising the nucleic acid SEQ ID NO. 109717, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 109717 or polypeptide SEQ ID NO. 109718, respectively, or a homolog or a fragment thereof, is increased or generated, or if the activity transcription factor is increased or generated in a non-human organism, like a microorganism or a plant cell, plant or part thereof, especially with non-targeted localization. For example, an increase of the linoleic acid of at least 1 percent, particularly in a range of 16 to 32-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization. For example, an increase of the linoleic acid of at least 1 percent, particularly in a range of 16 to 57-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a monothiol glutaredoxin, or if the activity of the polypeptide At4g15670, preferably represented by SEQ ID NO. 3280, or a homolog or fragment thereof, or if the activity of a polypeptide encoded by a nucleic acid molecule comprising the nucleic acid SEQ ID NO. 3279, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 3279 or polypeptide SEQ ID NO. 3280, respectively, or a homolog or a fragment thereof, is increased or generated, or if the activity monothiol glutaredoxin is increased or generated in a non-human organism, like a microorganism or a plant cell, plant or part thereof, especially with non-targeted localization. For example, an increase of the linoleic acid of at least 1 percent, particularly in a range of 21 to 59-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a protein phosphatase, or if the activity of the polypeptide At4g26080, preferably represented by SEQ ID NO. 68850, or a homolog or fragment thereof, or if the activity of a polypeptide encoded by a nucleic acid molecule comprising the nucleic acid SEQ ID NO. 68849, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 68849 or polypeptide SEQ ID NO. 68850, respectively, or a homolog or a fragment thereof, is increased or generated, or if the activity protein phosphatase is increased or generated in a non-human organism, like a microorganism or a plant cell, plant or part thereof, especially with non-targeted localization. For example, an increase of the linoleic acid of at least 1 percent, particularly in a range of 16 to 23-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a glutaredoxin, or if the activity of the polypeptide At4g33040, preferably represented by SEQ ID NO. 4103, or a homolog or fragment thereof, or if the activity of a polypeptide encoded by a nucleic acid molecule comprising the nucleic acid SEQ ID NO. 4102, preferably the coding region thereof, or a homolog or fragment thereof, e.g. derived from Arabidopsis thaliana, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 4102 or polypeptide SEQ ID NO. 4103, respectively, or a homolog or a fragment thereof, is increased or generated, or if the activity glutaredoxin is increased or generated in a non-human organism, like a microorganism or a plant cell, plant or part thereof, especially with non-targeted localization. For example, an increase of the linoleic acid of at least 1 percent, particularly in a range of 14 to 39-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a heat shock transcription factor, or if the activity of the polypeptide At5g03720, preferably represented by SEQ ID NO. 24439, or a homolog or fragment thereof, or if the activity of a polypeptide encoded by a nucleic acid molecule comprising the nucleic acid SEQ ID NO. 24438, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 24438 or polypeptide SEQ ID NO. 24439, respectively, or a homolog or a fragment thereof, is increased or generated, or if the activity heat shock transcription factor is increased or generated in a non-human organism, like a microorganism or a plant cell, plant or part thereof, especially with non-targeted localization. For example, an increase of the linoleic acid of at least 1 percent, particularly in a range of 22 to 42-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a protein phosphatase, or if the activity of the polypeptide At5g59220, preferably represented by SEQ ID NO. 25499, or a homolog or fragment thereof, or if the activity of a polypeptide encoded by a nucleic acid molecule comprising the nucleic acid SEQ ID NO. 25498, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 25498 or polypeptide SEQ ID NO. 25499, respectively, or a homolog or a fragment thereof, is increased or generated, or if the activity protein phosphatase is increased or generated in a non-human organism, like a microorganism or a plant cell, plant or part thereof, especially with non-targeted localization. For example, an increase of the linoleic acid of at least 1 percent, particularly in a range of 18 to 48-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a oxidoreductase, or if the activity of the polypeptide AvinDRAFT_{—}1788, preferably represented by SEQ ID NO. 114084, or a homolog or fragment thereof, or if the activity of a polypeptide encoded by a nucleic acid molecule comprising the nucleic acid SEQ ID NO. 114083, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 114083 or polypeptide SEQ ID NO. 114084, respectively, or a homolog or a fragment thereof, is increased or generated, or if the activity oxidoreductase is increased or generated in a non-human organism, like a microorganism or a plant cell, plant or part thereof, especially with non-targeted localization. For example, an increase of the linoleic acid of at least 1 percent, particularly in a range of 15 to 27-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a transcriptional regulator protein, or if the activity of the polypeptide AvinDRAFT_{—}2010, preferably represented by SEQ ID NO. 114199, or a homolog or fragment thereof, or if the activity of a polypeptide encoded by a nucleic acid molecule comprising the nucleic acid SEQ ID NO. 114198, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 114198 or polypeptide SEQ ID NO. 114199, respectively, or a homolog or a fragment thereof, is increased or generated, or if the activity transcriptional regulator protein is increased or generated in a non-human organism, like a microorganism or a plant cell, plant or part thereof, especially with non-targeted localization. For example, an increase of the linoleic acid of at least 1 percent, partitularly in a range of 15 to 38-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a beta-hydroxylase, or if the activity of the polypeptide AvinDRAFT_{—}2091, preferably represented by SEQ ID NO. 6041, or a homolog or fragment thereof, or if the activity of a polypeptide encoded by a nucleic acid molecule comprising the nucleic acid SEQ ID NO. 6040, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 6040 or polypeptide SEQ ID NO. 6041, respectively, or a homolog or a fragment thereof, is increased or generated, or if the activity beta-hydroxylase is increased or generated in a non-human organism, like a microorganism or a plant cell, plant or part thereof, especially with non-targeted localization. For example, an increase of the linoleic acid of at least 1 percent, particularly in a range of 18 to 29-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a oxidoreductase, or if the activity of the polypeptide AvinDRAFT_{—}3253, preferably represented by SEQ ID NO. 29501, or a homolog or fragment thereof, or if the activity of a polypeptide encoded by a nucleic acid molecule comprising the nucleic acid SEQ ID NO. 29500, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 29500 or polypeptide SEQ ID NO. 29501, respectively, or a homolog or a fragment thereof, is increased or generated, or if the activity oxidoreductase is increased or generated in a non-human organism, like a microorganism or a plant cell, plant or part thereof, especially with non-targeted localization. For example, an increase of the linoleic acid of at least 1 percent, particularly in a range of 14 to 44-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a ThiF family protein, or if the activity of the polypeptide AvinDRAFT_{—}3577, preferably represented by SEQ ID NO. 111156, or a homolog or fragment thereof, or if the activity of a polypeptide encoded by a nucleic acid molecule comprising the nucleic acid SEQ ID NO. 111155, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 111155 or polypeptide SEQ ID NO. 111156, respectively, or a homolog or a fragment thereof, is increased or generated, or if the activity ThiF family protein is increased or generated in a non-human organism, like a microorganism or a plant cell, plant or part thereof, especially with non-targeted localization. For example, an increase of the linoleic acid of at least 1 percent, particularly in a range of 15 to 43-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a recombinase A, or if the activity of the polypeptide AvinDRAFT_{—}3629, preferably represented by SEQ ID NO. 114232, or a homolog or fragment thereof, or if the activity of a polypeptide encoded by a nucleic acid molecule comprising the nucleic acid SEQ ID NO. 114231, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 114231 or polypeptide SEQ ID NO. 114232, respectively, or a homolog or a fragment thereof, is increased or generated, or if the activity recombinase A is increased or generated in a non-human organism, like a microorganism or a plant cell, plant or part thereof, especially with non-targeted localization. For example, an increase of the linoleic acid of at least 1 percent, particularly in a range of 16 to 30-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a hydrolase, or if the activity of the polypeptide AvinDRAFT_{—}5103, preferably represented by SEQ ID NO. 6511, or a homolog or fragment thereof, or if the activity of a polypeptide encoded by a nucleic acid molecule comprising the nucleic acid SEQ ID NO. 6510, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 6510 or polypeptide SEQ ID NO. 6511, respectively, or a homolog or a fragment thereof, is increased or generated, or if the activity hydrolase is increased or generated in a non-human organism, like a microorganism or a plant cell, plant or part thereof, especially with non-targeted localization. For example, an increase of the linoleic acid of at least 1 percent, particularly in a range of 20 to 36-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a membrane transport protein, or if the activity of the polypeptide B0486, preferably represented by SEQ ID NO. 7687, or a homolog or fragment thereof, or if the activity of a polypeptide encoded by a nucleic acid molecule comprising the nucleic acid SEQ ID NO. 7686, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 7686 or polypeptide SEQ ID NO. 7687, respectively, or a homolog or a fragment thereof, is increased or generated, or if the activity membrane transport protein is increased or generated in a non-human organism, like a microorganism or a plant cell, plant or part thereof, especially with non-targeted localization. For example, an increase of the linoleic acid of at least 1 percent, particularly in a range of 16 to 26-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a b0801-protein, or if the activity of the polypeptide B0801, preferably represented by SEQ ID NO. 75808, or a homolog or fragment thereof, or if the activity of a polypeptide encoded by a nucleic acid molecule comprising the nucleic acid SEQ ID NO. 75807, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 75807 or polypeptide SEQ ID NO. 75808, respectively, or a homolog or a fragment thereof, is increased or generated, or if the activity b0801-protein is increased or generated in a non-human organism, like a microorganism or a plant cell, plant or part thereof, especially with non-targeted localization. For example, an increase of the linoleic acid of at least 1 percent, particularly in a range of 15 to 30-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a 3-oxoacyl-[acylcarrier-protein] synthase, or if the activity of the polypeptide B1095, preferably represented by SEQ ID NO. 114553, or a homolog or fragment thereof, or if the activity of a polypeptide encoded by a nucleic acid molecule comprising the nucleic acid SEQ ID NO. 114552, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 114552 or polypeptide SEQ ID NO. 114553, respectively, or a homolog or a fragment thereof, is increased or generated, or if the activity 3-oxoacyl-[acyl-carrier-protein] synthase is increased or generated in a non-human organism, like a microorganism or a plant cell, plant or part thereof, especially with plastidic localization. For example, an increase of the linoleic acid of at least 1 percent, particularly in a range of 18 to 46-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a outer membrane receptor protein precursor, or if the activity of the polypeptide B1102, preferably represented by SEQ ID NO. 114980, or a homolog or fragment thereof, or if the activity of a polypeptide encoded by a nucleic acid molecule comprising the nucleic acid SEQ ID NO. 114979, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 114979 or polypeptide SEQ ID NO. 114980, respectively, or a homolog or a fragment thereof, is increased or generated, or if the activity outer membrane receptor protein precursor is increased or generated in a non-human organism, like a microorganism or a plant cell, plant or part thereof, especially with non-targeted localization. For example, an increase of the linoleic acid of at least 1 percent, particularly in a range of 15 to 21-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a b1330-protein, or if the activity of the polypeptide B1330, preferably represented by SEQ ID NO. 38227, or a homolog or fragment thereof, or if the activity of a polypeptide encoded by a nucleic acid molecule comprising the nucleic acid SEQ ID NO. 38226, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 38226 or polypeptide SEQ ID NO. 38227, respectively, or a homolog or a fragment thereof, is increased or generated, or if the activity b1330-protein is increased or generated in a non-human organism, like a microorganism or a plant cell, plant or part thereof, especially with non-targeted localization. For example, an increase of the linoleic acid of at least 1 percent, particularly in a range of 15 to 35-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a lipoprotein precursor, or if the activity of the polypeptide B1431, preferably represented by SEQ ID NO. 38267, or a homolog or fragment thereof, or if the activity of a polypeptide encoded by a nucleic acid molecule comprising the nucleic acid SEQ ID NO. 38266, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 38266 or polypeptide SEQ ID NO. 38267, respectively, or a homolog or a fragment thereof, is increased or generated, or if the activity lipoprotein precursor is increased or generated in a non-human organism, like a microorganism or a plant cell, plant or part thereof, especially with plastidic localization. For example, an increase of the linoleic acid of at least 1 percent, particularly in a range of 15 to 40-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a b1470-protein, or if the activity of the polypeptide B1470, preferably represented by SEQ ID NO. 115057, or a homolog or fragment thereof, or if the activity of a polypeptide encoded by a nucleic acid molecule comprising the nucleic acid SEQ ID NO. 115056, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 115056 or polypeptide SEQ ID NO. 115057, respectively, or a homolog or a fragment thereof, is increased or generated, or if the activity b1470-protein is increased or generated in a non-human organism, like a microorganism or a plant cell, plant or part thereof, especially with non-targeted localization. For example, an increase of the linoleic acid of at least 1 percent, particularly in a range of 14 to 39-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a Fe—S subunit of oxidoreductase, or if the activity of the polypeptide B1589, preferably represented by SEQ ID NO. 78155, or a homolog or fragment thereof, or if the activity of a polypeptide encoded by a nucleic acid molecule comprising the nucleic acid SEQ ID NO. 78154, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 78154 or polypeptide SEQ ID NO. 78155, respectively, or a homolog or a fragment thereof, is increased or generated, or if the activity Fe—S subunit of oxidoreductase is increased or generated in a non-human organism, like a microorganism or a plant cell, plant or part thereof, especially with non-targeted localization. For example, an increase of the linoleic acid of at least 1 percent, particularly in a range of 14 to 33-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a acid shock protein, or if the activity of the polypeptide B1597, preferably represented by SEQ ID NO. 38301, or a homolog or fragment thereof, or if the activity of a polypeptide encoded by a nucleic acid molecule comprising the nucleic acid SEQ ID NO. 38300, preferably the coding region thereof, or a homolog or fragment thereof, e.g. derived from Escherichia coli, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 38300 or polypeptide SEQ ID NO. 38301, respectively, or a homolog or a fragment thereof, is increased or generated, or if the activity acid shock protein is increased or generated in a non-human organism, like a microorganism or a plant cell, plant or part thereof, especially with non-targeted localization. For example, an increase of the linoleic acid of at least 1 percent, particularly in a range of 14 to 35-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a fumarate hydratase, or if the activity of the polypeptide B1611, preferably represented by SEQ ID NO. 115065, or a homolog or fragment thereof, or if the activity of a polypeptide encoded by a nucleic acid molecule comprising the nucleic acid SEQ ID NO. 115064, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 115064 or polypeptide SEQ ID NO. 115065, respectively, or a homolog or a fragment thereof, is increased or generated, or if the activity fumarate hydratase is increased or generated in a non-human organism, like a microorganism or a plant cell, plant or part thereof, especially with plastidic localization. For example, an increase of the linoleic acid of at least 1 percent, particularly in a range of 14 to 33-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a electron transport complex protein, or if the activity of the polypeptide B1627, preferably represented by SEQ ID NO. 38346, or a homolog or fragment thereof, or if the activity of a polypeptide encoded by a nucleic acid molecule comprising the nucleic acid SEQ ID NO. 38345, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 38345 or polypeptide SEQ ID NO. 38346, respectively, or a homolog or a fragment thereof, is increased or generated, or if the activity electron transport complex protein is increased or generated in a non-human organism, like a microorganism or a plant cell, plant or part thereof, especially with non-targeted localization. For example, an increase of the linoleic acid of at least 1 percent, particularly in a range of 18 to 25-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a b1670-protein, or if the activity of the polypeptide B1670, preferably represented by SEQ ID NO. 78754, or a homolog or fragment thereof, or if the activity of a polypeptide encoded by a nucleic acid molecule comprising the nucleic acid SEQ ID NO. 78753, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 78753 or polypeptide SEQ ID NO. 78754, respectively, or a homolog or a fragment thereof, is increased or generated, or if the activity b1670-protein is increased or generated in a non-human organism, like a microorganism or a plant cell, plant or part thereof, especially with non-targeted localization. For example, an increase of the linoleic acid of at least 1 percent, particularly in a range of 13 to 33-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a b1837-protein, or if the activity of the polypeptide B1837, preferably represented by SEQ ID NO. 78954, or a homolog or fragment thereof, or if the activity of a polypeptide encoded by a nucleic acid molecule comprising the nucleic acid SEQ ID NO. 78953, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 78953 or polypeptide SEQ ID NO. 78954, respectively, or a homolog or a fragment thereof, is increased or generated, or if the activity b1837-protein is increased or generated in a non-human organism, like a microorganism or a plant cell, plant or part thereof, especially with non-targeted localization. For example, an increase of the linoleic acid of at least 1 percent, particularly in a range of 13 to 37-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a b2032-protein, or if the activity of the polypeptide B2032, preferably represented by SEQ ID NO. 8921, or a homolog or fragment thereof, or if the activity of a polypeptide encoded by a nucleic acid molecule comprising the nucleic acid SEQ ID NO. 8920, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 8920 or polypeptide SEQ ID NO. 8921, respectively, or a homolog or a fragment thereof, is increased or generated, or if the activity b2032-protein is increased or generated in a non-human organism, like a microorganism or a plant cell, plant or part thereof, especially with non-targeted localization. For example, an increase of the linoleic acid of at least 1 percent, particularly in a range of 15 to 74-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a colanic acid biosynthesis protein, or if the activity of the polypeptide B2043, preferably represented by SEQ ID NO. 112194, or a homolog or fragment thereof, or if the activity of a polypeptide encoded by a nucleic acid molecule comprising the nucleic acid SEQ ID NO. 112193, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 112193 or polypeptide SEQ ID NO. 112194, respectively, or a homolog or a fragment thereof, is increased or generated, or if the activity colanic acid biosynthesis protein is increased or generated in a non-human organism, like a microorganism or a plant cell, plant or part thereof, especially with non-targeted localization. For example, an increase of the linoleic acid of at least 1 percent, particularly in a range of 14 to 27-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a b2099-protein, or if the activity of the polypeptide B2099, preferably represented by SEQ ID NO. 115596, or a homolog or fragment thereof, or if the activity of a polypeptide encoded by a nucleic acid molecule comprising the nucleic acid SEQ ID NO. 115595, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 115595 or polypeptide SEQ ID NO. 115596, respectively, or a homolog or a fragment thereof, is increased or generated, or if the activity b2099-protein is increased or generated in a non-human organism, like a microorganism or a plant cell, plant or part thereof, especially with non-targeted localization. For example, an increase of the linoleic acid of at least 1 percent, particularly in a range of 16 to 31-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a b2107-protein, or if the activity of the polypeptide B2107, preferably represented by SEQ ID NO. 39003, or a homolog or fragment thereof, or if the activity of a polypeptide encoded by a nucleic acid molecule comprising the nucleic acid SEQ ID NO. 39002, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 39002 or polypeptide SEQ ID NO. 39003, respectively, or a homolog or a fragment thereof, is increased or generated, or if the activity b2107-protein is increased or generated in a non-human organism, like a microorganism or a plant cell, plant or part thereof, especially with non-targeted localization. For example, an increase of the linoleic acid of at least 1 percent, particularly in a range of 15 to 47-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a ABC transporter permease protein, or if the activity of the polypeptide B2178, preferably represented by SEQ ID NO. 39041, or a homolog or fragment thereof, or if the activity of a polypeptide encoded by a nucleic acid molecule comprising the nucleic acid SEQ ID NO. 39040, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 39040 or polypeptide SEQ ID NO. 39041, respectively, or a homolog or a fragment thereof, is increased or generated, or if the activity ABC transporter permease protein is increased or generated in a non-human organism, like a microorganism or a plant cell, plant or part thereof, especially with non-targeted localization. For example, an increase of the linoleic acid of at least 1 percent, particularly in a range of 16 to 40-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a b2399-protein, or if the activity of the polypeptide B2399, preferably represented by SEQ ID NO. 39238, or a homolog or fragment thereof, or if the activity of a polypeptide encoded by a nucleic acid molecule comprising the nucleic acid SEQ ID NO. 39237, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 39237 or polypeptide SEQ ID NO. 39238, respectively, or a homolog or a fragment thereof, is increased or generated, or if the activity b2399-protein is increased or generated in a non-human organism, like a microorganism or a plant cell, plant or part thereof, especially with non-targeted localization. For example, an increase of the linoleic acid of at least 1 percent, particularly in a range of 24 to 35-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a ethanolamine utilization protein, or if the activity of the polypeptide B2439, preferably represented by SEQ ID NO. 115629, or a homolog or fragment thereof, or if the activity of a polypeptide encoded by a nucleic acid molecule comprising the nucleic acid SEQ ID NO. 115628, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 115628 or polypeptide SEQ ID NO. 115629, respectively, or a homolog or a fragment thereof, is increased or generated, or if the activity ethanolamine utilization protein is increased or generated in a non-human organism, like a microorganism or a plant cell, plant or part thereof, especially with non-targeted localization. For example, an increase of the linoleic acid of at least 1 percent, particularly in a range of 20 to 34-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a b2474-protein, or if the activity of the polypeptide B2474, preferably represented by SEQ ID NO. 40330, or a homolog or fragment thereof, or if the activity of a polypeptide encoded by a nucleic acid molecule comprising the nucleic acid SEQ ID NO. 40329, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 40329 or polypeptide SEQ ID NO. 40330, respectively, or a homolog or a fragment thereof, is increased or generated, or if the activity b2474-protein is increased or generated in a non-human organism, like a microorganism or a plant cell, plant or part thereof, especially with non-targeted localization. For example, an increase of the linoleic acid of at least 1 percent, particularly in a range of 19 to 39-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a b2613-protein, or if the activity of the polypeptide B2613, preferably represented by SEQ ID NO. 40666, or a homolog or fragment thereof, or if the activity of a polypeptide encoded by a nucleic acid molecule comprising the nucleic acid SEQ ID NO. 40665, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 40665 or polypeptide SEQ ID NO. 40666, respectively, or a homolog or a fragment thereof, is increased or generated, or if the activity b2613-protein is increased or generated in a non-human organism, like a microorganism or a plant cell, plant or part thereof, especially with non-targeted localization. For example, an increase of the linoleic acid of at least 1 percent, particularly in a range of 14 to 48-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a b2682-protein, or if the activity of the polypeptide B2682, preferably represented by SEQ ID NO. 115656, or a homolog or fragment thereof, or if the activity of a polypeptide encoded by a nucleic acid molecule comprising the nucleic acid SEQ ID NO. 115655, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 115655 or polypeptide SEQ ID NO. 115656, respectively, or a homolog or a fragment thereof, is increased or generated, or if the activity b2682-protein is increased or generated in a non-human organism, like a microorganism or a plant cell, plant or part thereof, especially with non-targeted localization. For example, an increase of the linoleic acid of at least 1 percent, particularly in a range of 20 to 23-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a flavodoxin, or if the activity of the polypeptide B2790, preferably represented by SEQ ID NO. 115720, or a homolog or fragment thereof, or if the activity of a polypeptide encoded by a nucleic acid molecule comprising the nucleic acid SEQ ID NO. 115719, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 115719 or polypeptide SEQ ID NO. 115720, respectively, or a homolog or a fragment thereof, is increased or generated, or if the activity flavodoxin is increased or generated in a non-human organism, like a microorganism or a plant cell, plant or part thereof, especially with non-targeted localization. For example, an increase of the linoleic acid of at least 1 percent, particularly in a range of 17 to 25-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a AAS bifunctional protein, or if the activity of the polypeptide B2836, preferably represented by SEQ ID NO. 115792, or a homolog or fragment thereof, or if the activity of a polypeptide encoded by a nucleic acid molecule comprising the nucleic acid SEQ ID NO. 115791, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 115791 or polypeptide SEQ ID NO. 115792, respectively, or a homolog or a fragment thereof, is increased or generated, or if the activity AAS bifunctional protein is increased or generated in a non-human organism, like a microorganism or a plant cell, plant or part thereof, especially with plastidic localization. For example, an increase of the linoleic acid of at least 1 percent, particularly in a range of 19 to 43-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a b2849-protein, or if the activity of the polypeptide B2849, preferably represented by SEQ ID NO. 81981, or a homolog or fragment thereof, or if the activity of a polypeptide encoded by a nucleic acid molecule comprising the nucleic acid SEQ ID NO. 81980, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 81980 or polypeptide SEQ ID NO. 81981, respectively, or a homolog or a fragment thereof, is increased or generated, or if the activity b2849-protein is increased or generated in a non-human organism, like a microorganism or a plant cell, plant or part thereof, especially with non-targeted localization. For example, an increase of the linoleic acid of at least 1 percent, particularly in a range of 15 to 40-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a xanthine dehydrogenase, or if the activity of the polypeptide B2866, preferably represented by SEQ ID NO. 115865, or a homolog or fragment thereof, or if the activity of a polypeptide encoded by a nucleic acid molecule comprising the nucleic acid SEQ ID NO. 115864, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 115864 or polypeptide SEQ ID NO. 115865, respectively, or a homolog or a fragment thereof, is increased or generated, or if the activity xanthine dehydrogenase is increased or generated in a non-human organism, like a microorganism or a plant cell, plant or part thereof, especially with non-targeted localization. For example, an increase of the linoleic acid of at least 1 percent, particularly in a range of 16 to 20-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a b2909-protein, or if the activity of the polypeptide B2909, preferably represented by SEQ ID NO. 41007, or a homolog or fragment thereof, or if the activity of a polypeptide encoded by a nucleic acid molecule comprising the nucleic acid SEQ ID NO. 41006, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 41006 or polypeptide SEQ ID NO. 41007, respectively, or a homolog or a fragment thereof, is increased or generated, or if the activity b2909-protein is increased or generated in a non-human organism, like a microorganism or a plant cell, plant or part thereof, especially with non-targeted localization. For example, an increase of the linoleic acid of at least 1 percent, particularly in a range of 20 to 28-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a phosphotriesterase, or if the activity of the polypeptide B3379, preferably represented by SEQ ID NO. 116111, or a homolog or fragment thereof, or if the activity of a polypeptide encoded by a nucleic acid molecule comprising the nucleic acid SEQ ID NO. 116110, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 116110 or polypeptide SEQ ID NO. 116111, respectively, or a homolog or a fragment thereof, is increased or generated, or if the activity phosphotriesterase is increased or generated in a non-human organism, like a microorganism or a plant cell, plant or part thereof, especially with non-targeted localization. For example, an increase of the linoleic acid of at least 1 percent, particularly in a range of 16 to 34-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a sn-glycerol-3-phosphate transport system permease protein, or if the activity of the polypeptide B3451, preferably represented by SEQ ID NO. 116173, or a homolog or fragment thereof, or if the activity of a polypeptide encoded by a nucleic acid molecule comprising the nucleic acid SEQ ID NO. 116172, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 116172 or polypeptide SEQ ID NO. 116173, respectively, or a homolog or a fragment thereof, is increased or generated, or if the activity sn-glycerol-3-phosphate transport system permease protein is increased or generated in a non-human organism, like a microorganism or a plant cell, plant or part thereof, especially with plastidic localization. For example, an increase of the linoleic acid of at least 1 percent, particularly in a range of 14 to 32-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a threonine efflux protein, or if the activity of the polypeptide B3823, preferably represented by SEQ ID NO. 10727, or a homolog or fragment thereof, or if the activity of a polypeptide encoded by a nucleic acid molecule comprising the nucleic acid SEQ ID NO. 10726, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 10726 or polypeptide SEQ ID NO. 10727, respectively, or a homolog or a fragment thereof, is increased or generated, or if the activity threonine efflux protein is increased or generated in a non-human organism, like a microorganism or a plant cell, plant or part thereof, especially with non-targeted localization. For example, an increase of the linoleic acid of at least 1 percent, particularly in a range of 14 to 42-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a 3-ketoacyl-CoA thiolase, or if the activity of the polypeptide B3845, preferably represented by SEQ ID NO. 100676, or a homolog or fragment thereof, or if the activity of a polypeptide encoded by a nucleic acid molecule comprising the nucleic acid SEQ ID NO. 100675, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 100675 or polypeptide SEQ ID NO. 100676, respectively, or a homolog or a fragment thereof, is increased or generated, or if the activity 3-ketoacyl-CoA thiolase is increased or generated in a non-human organism, like a microorganism or a plant cell, plant or part thereof, especially with plastidic localization. For example, an increase of the linoleic acid of at least 1 percent, particularly in a range of 11 to 29-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a GM02LC38418-protein, or if the activity of the polypeptide GM02LC38418, preferably represented by SEQ ID NO. 116313, or a homolog or fragment thereof, or if the activity of a polypeptide encoded by a nucleic acid molecule comprising the nucleic acid SEQ ID NO. 116312, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 116312 or polypeptide SEQ ID NO. 116313, respectively, or a homolog or a fragment thereof, is increased or generated, or if the activity GM02LC38418-protein is increased or generated in a non-human organism, like a microorganism or a plant cell, plant or part thereof, especially with non-targeted localization. For example, an increase of the linoleic acid of at least 1 percent, particularly in a range of 14 to 23-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a sll1032-protein, or if the activity of the polypeptide Sll1032, preferably represented by SEQ ID NO. 85724, or a homolog or fragment thereof, or if the activity of a polypeptide encoded by a nucleic acid molecule comprising the nucleic acid SEQ ID NO. 85723, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 85723 or polypeptide SEQ ID NO. 85724, respectively, or a homolog or a fragment thereof, is increased or generated, or if the activity sll1032-protein is increased or generated in a non-human organism, like a microorganism or a plant cell, plant or part thereof, especially with mitochondrial localization. For example, an increase of the linoleic acid of at least 1 percent, particularly in a range of 16 to 22-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a phosphoribosylformyl glycinamidine synthase subunit, or if the activity of the polypeptide Sll1056, preferably represented by SEQ ID NO. 52661, or a homolog or fragment thereof, or if the activity of a polypeptide encoded by a nucleic acid molecule comprising the nucleic acid SEQ ID NO. 52660, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 52660 or polypeptide SEQ ID NO. 52661, respectively, or a homolog or a fragment thereof, is increased or generated, or if the activity phosphoribosylformyl glycinamidine synthase subunit is increased or generated in a non-human organism, like a microorganism or a plant cell, plant or part thereof, especially with plastidic localization. For example, an increase of the linoleic acid of at least 1 percent, particularly in a range of 18 to 40-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a CTP synthetase, or if the activity of the polypeptide Sll1443, preferably represented by SEQ ID NO. 53879, or a homolog or fragment thereof, or if the activity of a polypeptide encoded by a nucleic acid molecule comprising the nucleic acid SEQ ID NO. 53878, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 53878 or polypeptide SEQ ID NO. 53879, respectively, or a homolog or a fragment thereof, is increased or generated, or if the activity CTP synthetase is increased or generated in a non-human organism, like a microorganism or a plant cell, plant or part thereof, especially with mitochondrial localization. For example, an increase of the linoleic acid of at least 1 percent, particularly in a range of 15 to 33-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a carbamoyl-phosphate synthase subunit, or if the activity of the polypeptide Sll1498, preferably represented by SEQ ID NO. 95048, or a homolog or fragment thereof, or if the activity of a polypeptide encoded by a nucleic acid molecule comprising the nucleic acid SEQ ID NO. 95047, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 95047 or polypeptide SEQ ID NO. 95048, respectively, or a homolog or a fragment thereof, is increased or generated, or if the activity carbamoyl-phosphate synthase subunit is increased or generated in a non-human organism, like a microorganism or a plant cell, plant or part thereof, especially with non-targeted localization. For example, an increase of the linoleic acid of at least 1 percent, particularly in a range of 18 to 34-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a coproporphyrinogen III oxidase, or if the activity of the polypeptide Sll1917, preferably represented by SEQ ID NO. 11472, or a homolog or fragment thereof, or if the activity of a polypeptide encoded by a nucleic acid molecule comprising the nucleic acid SEQ ID NO. 11471, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 11471 or polypeptide SEQ ID NO. 11472, respectively, or a homolog or a fragment thereof, is increased or generated, or if the activity coproporphyrinogen III oxidase is increased or generated in a non-human organism, like a microorganism or a plant cell, plant or part thereof, especially with mitochondrial localization. For example, an increase of the linoleic acid of at least 1 percent, particularly in a range of 14 to 29-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a cationtransporting ATPase, or if the activity of the polypeptide Sll1920, preferably represented by SEQ ID NO. 55386, or a homolog or fragment thereof, or if the activity of a polypeptide encoded by a nucleic acid molecule comprising the nucleic acid SEQ ID NO. 55385, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 55385 or polypeptide SEQ ID NO. 55386, respectively, or a homolog or a fragment thereof, is increased or generated, or if the activity cation-transporting ATPase is increased or generated in a non-human organism, like a microorganism or a plant cell, plant or part thereof, especially with mitochondrial localization. For example, an increase of the linoleic acid of at least 1 percent, particularly in a range of 14 to 22-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a oxidoreductase, or if the activity of the polypeptide Slr0338, preferably represented by SEQ ID NO. 11991, or a homolog or fragment thereof, or if the activity of a polypeptide encoded by a nucleic acid molecule comprising the nucleic acid SEQ ID NO. 11990, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 11990 or polypeptide SEQ ID NO. 11991, respectively, or a homolog or a fragment thereof, is increased or generated, or if the activity oxidoreductase is increased or generated in a non-human organism, like a microorganism or a plant cell, plant or part thereof, especially with non-targeted localization. For example, an increase of the linoleic acid of at least 1 percent, particularly in a range of 14 to 42-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a tryptophan synthase alpha chain, or if the activity of the polypeptide Slr0966, preferably represented by SEQ ID NO. 87656, or a homolog or fragment thereof, or if the activity of a polypeptide encoded by a nucleic acid molecule comprising the nucleic acid SEQ ID NO. 87655, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 87655 or polypeptide SEQ ID NO. 87656, respectively, or a homolog or a fragment thereof, is increased or generated, or if the activity tryptophan synthase alpha chain is increased or generated in a non-human organism, like a microorganism or a plant cell, plant or part thereof, especially with plastidic localization. For example, an increase of the linoleic acid of at least 1 percent, particularly in a range of 17 to 43-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a aromatic acid decarboxylase, or if the activity of the polypeptide Slr1099, preferably represented by SEQ ID NO. 116342, or a homolog or fragment thereof, or if the activity of a polypeptide encoded by a nucleic acid molecule comprising the nucleic acid SEQ ID NO. 116341, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 116341 or polypeptide SEQ ID NO. 116342, respectively, or a homolog or a fragment thereof, is increased or generated, or if the activity aromatic acid decarboxylase is increased or generated in a non-human organism, like a microorganism or a plant cell, plant or part thereof, especially with mitochondrial localization. For example, an increase of the linoleic acid of at least 1 percent, particularly in a range of 14 to 44-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a phosphate import ATP-binding protein, or if the activity of the polypeptide Slr1250, preferably represented by SEQ ID NO. 116461, or a homolog or fragment thereof, or if the activity of a polypeptide encoded by a nucleic acid molecule comprising the nucleic acid SEQ ID NO. 116460, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 116460 or polypeptide SEQ ID NO. 116461, respectively, or a homolog or a fragment thereof, is increased or generated, or if the activity phosphate import ATP-binding protein is increased or generated in a non-human organism, like a microorganism or a plant cell, plant or part thereof, especially with mitochondrial localization. For example, an increase of the linoleic acid of at least 1 percent, particularly in a range of 14 to 29-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a gamma-glutamyltranspeptidase, or if the activity of the polypeptide Slr1269, preferably represented by SEQ ID NO. 58059, or a homolog or fragment thereof, or if the activity of a polypeptide encoded by a nucleic acid molecule comprising the nucleic acid SEQ ID NO. 58058, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 58058 or polypeptide SEQ ID NO. 58059, respectively, or a homolog or a fragment thereof, is increased or generated, or if the activity gamma-glutamyltranspeptidase is increased or generated in a non-human organism, like a microorganism or a plant cell, plant or part thereof, especially with non-targeted localization. For example, an increase of the linoleic acid of at least 1 percent, particularly in a range of 18 to 24-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a arginine decarboxylase, or if the activity of the polypeptide Slr1312, preferably represented by SEQ ID NO. 58325, or a homolog or fragment thereof, or if the activity of a polypeptide encoded by a nucleic acid molecule comprising the nucleic acid SEQ ID NO. 58324, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 58324 or polypeptide SEQ ID NO. 58325, respectively, or a homolog or a fragment thereof, is increased or generated, or if the activity arginine decarboxylase is increased or generated in a non-human organism, like a microorganism or a plant cell, plant or part thereof, especially with non-targeted localization. For example, an increase of the linoleic acid of at least 1 percent, particularly in a range of 14 to 45-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a fatty acid desaturase, or if the activity of the polypeptide Slr1350, preferably represented by SEQ ID NO. 116714, or a homolog or fragment thereof, or if the activity of a polypeptide encoded by a nucleic acid molecule comprising the nucleic acid SEQ ID NO. 116713, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 116713 or polypeptide SEQ ID NO. 116714, respectively, or a homolog or a fragment thereof, is increased or generated, or if the activity fatty acid desaturase is increased or generated in a non-human organism, like a microorganism or a plant cell, plant or part thereof, especially with non-targeted localization. For example, an increase of the linoleic acid of at least 1 percent, particularly in a range of 14 to 29-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a threonine dehydratase, or if the activity of the polypeptide Slr2072, preferably represented by SEQ ID NO. 12342, or a homolog or fragment thereof, or if the activity of a polypeptide encoded by a nucleic acid molecule comprising the nucleic acid SEQ ID NO. 12341, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 12341 or polypeptide SEQ ID NO. 12342, respectively, or a homolog or a fragment thereof, is increased or generated, or if the activity threonine dehydratase is increased or generated in a non-human organism, like a microorganism or a plant cell, plant or part thereof, especially with mitochondrial localization. For example, an increase of the linoleic acid of at least 1 percent, particularly in a range of 13 to 35-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a TTC0490-protein, or if the activity of the polypeptide TTC0490, preferably represented by SEQ ID NO. 116849, or a homolog or fragment thereof, or if the activity of a polypeptide encoded by a nucleic acid molecule comprising the nucleic acid SEQ ID NO. 116848, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 116848 or polypeptide SEQ ID NO. 116849, respectively, or a homolog or a fragment thereof, is increased or generated, or if the activity TTC0490-protein is increased or generated in a non-human organism, like a microorganism or a plant cell, plant or part thereof, especially with non-targeted localization.

For example, an increase of the linoleic acid of at least 1 percent, particularly in a range of 16 to 31-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a metal-dependent hydrolase, or if the activity of the polypeptide TTC0917, preferably represented by SEQ ID NO. 61554, or a homolog or fragment thereof, or if the activity of a polypeptide encoded by a nucleic acid molecule comprising the nucleic acid SEQ ID NO. 61553, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 61553 or polypeptide SEQ ID NO. 61554, respectively, or a homolog or a fragment thereof, is increased or generated, or if the activity metal-dependent hydrolase is increased or generated in a non-human organism, like a microorganism or a plant cell, plant or part thereof, especially with non-targeted localization. For example, an increase of the linoleic acid of at least 1 percent, particularly in a range of 23 to 98-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a multiple antibiotic resistance protein, or if the activity of the polypeptide TTC1193, preferably represented by SEQ ID NO. 61724, or a homolog or fragment thereof, or if the activity of a polypeptide encoded by a nucleic acid molecule comprising the nucleic acid SEQ ID NO. 61723, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 61723 or polypeptide SEQ ID NO. 61724, respectively, or a homolog or a fragment thereof, is increased or generated, or if the activity multiple antibiotic resistance protein is increased or generated in a non-human organism, like a microorganism or a plant cell, plant or part thereof, especially with non-targeted localization. For example, an increase of the linoleic acid of at least 1 percent, particularly in a range of 17 to 22-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a glycin cleavage system protein, or if the activity of the polypeptide Yal044c, preferably represented by SEQ ID NO. 116949, or a homolog or fragment thereof, or if the activity of a polypeptide encoded by a nucleic acid molecule comprising the nucleic acid SEQ ID NO. 116948, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 116948 or polypeptide SEQ ID NO. 116949, respectively, or a homolog or a fragment thereof, is increased or generated, or if the activity glycin cleavage system protein is increased or generated in a non-human organism, like a microorganism or a plant cell, plant or part thereof, especially with plastidic localization. For example, an increase of the linoleic acid of at least 1 percent, particularly in a range of 17 to 29-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a protein kinase, or if the activity of the polypeptide Ydl159w, preferably represented by SEQ ID NO. 14276, or a homolog or fragment thereof, or if the activity of a polypeptide encoded by a nucleic acid molecule comprising the nucleic acid SEQ ID NO. 14275, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 14275 or polypeptide SEQ ID NO. 14276, respectively, or a homolog or a fragment thereof, is increased or generated, or if the activity protein kinase is increased or generated in a non-human organism, like a microorganism or a plant cell, plant or part thereof, especially with plastidic localization. For example, an increase of the linoleic acid of at least 1 percent, particularly in a range of 16 to 34-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a aspartatesemialdehyde dehydrogenase, or if the activity of the polypeptide Ydr158w, preferably represented by SEQ ID NO. 117353, or a homolog or fragment thereof, or if the activity of a polypeptide encoded by a nucleic acid molecule comprising the nucleic acid SEQ ID NO. 117352, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 117352 or polypeptide SEQ ID NO. 117353, respectively, or a homolog or a fragment thereof, is increased or generated, or if the activity aspartate-semialdehyde dehydrogenase is increased or generated in a non-human organism, like a microorganism or a plant cell, plant or part thereof, especially with plastidic localization. For example, an increase of the linoleic acid of at least 1 percent, particularly in a range of 14 to 38-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a ydr507c-protein, or if the activity of the polypeptide Ydr507c, preferably represented by SEQ ID NO. 90166, or a homolog or fragment thereof, or if the activity of a polypeptide encoded by a nucleic acid molecule comprising the nucleic acid SEQ ID NO. 90165, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 90165 or polypeptide SEQ ID NO. 90166, respectively, or a homolog or a fragment thereof, is increased or generated, or if the activity ydr507c-protein is increased or generated in a non-human organism, like a microorganism or a plant cell, plant or part thereof, especially with non-targeted localization. For example, an increase of the linoleic acid of at least 1 percent, particularly in a range of 14 to 34-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a YGR104C-protein, or if the activity of the polypeptide Ygr104c, preferably represented by SEQ ID NO. 117483, or a homolog or fragment thereof, or if the activity of a polypeptide encoded by a nucleic acid molecule comprising the nucleic acid SEQ ID NO. 117482, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 117482 or polypeptide SEQ ID NO. 117483, respectively, or a homolog or a fragment thereof, is increased or generated, or if the activity YGR104C-protein is increased or generated in a non-human organism, like a microorganism or a plant cell, plant or part thereof, especially with non-targeted localization. For example, an increase of the linoleic acid of at least 1 percent, particularly in a range of 15 to 19-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a DnaJ-like chaperone, or if the activity of the polypeptide Yj1073w, preferably represented by SEQ ID NO. 64965, or a homolog or fragment thereof, or if the activity of a polypeptide encoded by a nucleic acid molecule comprising the nucleic acid SEQ ID NO. 64964, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 64964 or polypeptide SEQ ID NO. 64965, respectively, or a homolog or a fragment thereof, is increased or generated, or if the activity DnaJ-like chaperone is increased or generated in a non-human organism, like a microorganism or a plant cell, plant or part thereof, especially with plastidic localization. For example, an increase of the linoleic acid of at least 1 percent, particularly in a range of 14 to 21-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a aconitate hydratase, or if the activity of the polypeptide Ylr304c, preferably represented by SEQ ID NO. 66773, or a homolog or fragment thereof, or if the activity of a polypeptide encoded by a nucleic acid molecule comprising the nucleic acid SEQ ID NO. 66772, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 66772 or polypeptide SEQ ID NO. 66773, respectively, or a homolog or a fragment thereof, is increased or generated, or if the activity aconitate hydratase is increased or generated in a non-human organism, like a microorganism or a plant cell, plant or part thereof, especially with non-targeted localization. For example, an increase of the linoleic acid of at least 1 percent, particularly in a range of 16 to 34-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a molecular chaperone, or if the activity of the polypeptide Ynl064c, preferably represented by SEQ ID NO. 117496, or a homolog or fragment thereof, or if the activity of a polypeptide encoded by a nucleic acid molecule comprising the nucleic acid SEQ ID NO. 117495, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 117495 or polypeptide SEQ ID NO. 117496, respectively, or a homolog or a fragment thereof, is increased or generated, or if the activity molecular chaperone is increased or generated in a non-human organism, like a microorganism or a plant cell, plant or part thereof, especially with non-targeted localization. For example, an increase of the linoleic acid of at least 1 percent, particularly in a range of 15 to 25-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a ATPase epsilon subunit, or if the activity of the polypeptide Ypl271w, preferably represented by SEQ ID NO. 118213, or a homolog or fragment thereof, or if the activity of a polypeptide encoded by a nucleic acid molecule comprising the nucleic acid SEQ ID NO. 118212, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 118212 or polypeptide SEQ ID NO. 118213, respectively, or a homolog or a fragment thereof, is increased or generated, or if the activity ATPase epsilon subunit is increased or generated in a non-human organism, like a microorganism or a plant cell, plant or part thereof, especially with plastidic localization. For example, an increase of the linoleic acid of at least 1 percent, particularly in a range of 14 to 34-percent is conferred as compared to a corresponding non-transformed wild type non-human organism.

Accordingly, in one embodiment, an increase of linoleic acid in a non-human organism, as compared to a corresponding non-transformed wild type non-human organism, is conferred in the process of the invention, if the activity of a polypeptide showing the activity of a glucose-6-phosphate 1-dehydrogenase, or if the activity of the polypeptide 59582753_SOYBEAN, preferably represented by SEQ ID NO. 16884, or a homolog or fragment thereof, or if the activity of a polypeptide encoded by a nucleic acid molecule comprising the nucleic acid SEQ ID NO. 16883, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 8, column 5 or 8 in the respective same line as the nucleic acid molecule SEQ ID NO. 16883 or polypeptide SEQ ID NO. 16884, respectively, or a homolog or a fragment thereof, is increased or generated, or if the activity glucose-6-phosphate 1-dehydrogenase is increased or generated in a non-human organism, like a microorganism or a plant cell, plant or part thereof, especially with plastidic localization. For example, an increase of the linoleic acid of at least 1 percent, particularly in a range of 14 to 37-percent is conferred as compared to a corresponding non-transformed wild type non-human organism.

In an embodiment of the invention, a protein or polypeptide has the “activity of a protein as shown in the respective line in Table II, column 3” if its de novo activity, or its increased expression directly or indirectly leads to an increase in the fine chemical level in the organism or a part thereof, preferably in a cell of said organism, and the protein has the above-mentioned activities of a protein as shown in the respective line in Table II, column 3. Throughout the specification the activity or preferably the biological activity of such a protein or polypeptide or an nucleic acid molecule or sequence encoding such protein or polypeptide is identical or similar if it still has the biological or enzymatic activity of a protein as shown in the respective line in Table II, column 3, or which has at least 10% of the original biological or enzymatic activity, preferably at least 20%, 30%, 40%, 50%, 60%, 70%, 80%, 90%, 95% or 99% in comparison to a protein as shown in Table II, column 3.

A series of mechanisms exists via which a modification of a protein, e.g. the polypeptide of the invention can directly or indirectly affect the yield, production and/or production efficiency of the fine chemical.

For example, the molecule number or the specific activity of the polypeptide or the nucleic acid molecule may be increased. Larger amounts of the fine chemical can be produced if the polypeptide or the nucleic acid of the invention is expressed de novo in an organism lacking the activity of said protein, preferably the nucleic acid molecules as mentioned in the respective line in Table I, columns 5 or 8, preferably the coding region thereof, or a homolog or a fragment thereof, alone or, if desired in combination with a transit peptide for example as mentioned in Table a or b, respectively, or in another embodiment by introducing said nucleic acid molecules into an organelle, such as a plastid, in the transgenic organism. However, it is also possible to modify the expression of the gene which is naturally present in the organisms, for example by integrating a nucleic acid sequence, encoding an organelle targeting sequence in front (5 prime) of the coding sequence, leading to a functional preprotein, which is directed for example to the organelle.

This also applies analogously to the combined increased expression of the nucleic acid molecule of the present invention or its gene product with that of further enzymes or regulators of the fine chemical biosynthesis pathways, e.g. which are useful for the synthesis of the fine chemicals.

The increase or modulation according to this invention can be constitutive, e.g. due to a stable permanent transgenic expression or to a stable mutation in the corresponding endogenous gene encoding the nucleic acid molecule of the invention or to a modulation of the expression or of the behaviour of a gene conferring the expression of the polypeptide of the invention, or transient, e.g. due to a transient transformation or temporary addition of a modulafor such as an agonist or antagonist or inducible, e.g. after transformation with a inducible construct carrying the nucleic acid molecule of the invention under control of an inducible promoter and adding the inducer, e.g. tetracycline or as described herein below.

The increase in activity of the polypeptide amounts in a cell, an organelle, an organ or a non-human organism or a part thereof preferably to at least 1%, 5%, 10%, 20%, 50%, 70%, 80%, 90%, 100%, 150% or 200%, 250% or more in comparison to the control, reference or wild type.

In an embodiment the increase in activity of the polypeptide amounts in an organelle such as a plastid or a mitochondrion. In another embodiment the increase in activity of the polypeptide amounts in the cytoplasm. In another embodiment the increase in activity of the polypeptide amounts in the cytosol.

In the context of the application the term “cytoplasm” defines the compartment of the cell to which the protein is directed by the host cell without the addition of artificial targeting sequences, which are naturally not part of the protein.

In the context of the application the term “cytosol” defines the fluid of the cell between the plasma membrane and the organelles; that means that the organelles as well as the nucleus are not part of the cytosol.

The specific activity of a polypeptide encoded by a nucleic acid molecule of the present invention or of the polypeptide of the present invention can be tested as described in the examples. In particular, the expression of a protein in question in a cell, e.g. a plant cell or a microorganism and the detection of an increase the fine chemical level in comparison to a control is an easy test and can be performed as described in the state of the art.

In this paragraph “A” means “Goffeau et al., Science 274 (5287), 546 (1996)”, “B” means “Blattner et al., Science 277 (5331), 1453 (1997)”, “C” means “Nakamura et al., Nucleic Acids Res. 28(1), 72 (2000)”, “D” means “deposition of the sequences to the EMBL/GenBank/DDBJ databases under “Sequencing of the draft genome assembly of Azotobacter vinelandii AvOP”. in June 2005 by Copeland et al.”, “E” means “Henne et al., Nat. Biotechnol. 22 (5), 547 (2004)”, “F” means “Kaul et al., Nature 408 (6814), 796 (2000)”, and “G” means “Matsumoto et al., Nature 436 (7052), 793 (2005)”.

The nucleic acid sequence of At1g19670 from Arabidopsis thaliana, e.g. as shown in the respective line in column 5 of Table I, application no. 8, has been published in F. And the activity of the gene product thereof is the activity of chlorophyllase.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “chlorophyllase”, especially from Arabidopsis thaliana or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said At1g19670, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said At1g19670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said At1g19670, or a functional equivalent or a homolog thereof as depicted in column 8 of Table II, application no. 8, preferably a homolog or functional equivalent as depicted in column 8 of Table II B, application no. 8, and being depicted in the same respective line as said At1g19670, and preferably the activity is increased non-targeted.

Accordingly, in one embodiment, the molecule which activity is to be increased in the process of the invention is the gene product with an activity as a “chlorophyllase”, preferably it is the molecule of section (a) or (b) of this paragraph.

In particular, it was observed that in plants, especially in Arabidopsis thaliana, increasing or generating the activity of a gene product non-targeted with the activity of a “chlorophyllase”, preferably being encoded by a gene comprising the nucleic acid sequence SEQ ID NO. 113651, preferably the coding region thereof, conferred the production of or the increase in linoleic acid compared with the wild type control.

The nucleic acid sequence of At1g19800 from Arabidopsis thaliana, e.g. as shown in the respective line in column 5 of Table I, application no. 8, has been published in F. And the activity of the gene product thereof is the activity of At1g19800-protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “At1g19800-protein”, especially from Arabidopsis thaliana or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said At1g19800, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said At1g19800, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said At1g19800, or a functional equivalent or a homolog thereof as depicted in column 8 of Table II, application no. 8, preferably a homolog or functional equivalent as depicted in column 8 of Table II B, application no. 8, and being depicted in the same respective line as said At1g19800, and preferably the activity is increased non-targeted.

Accordingly, in one embodiment, the molecule which activity is to be increased in the process of the invention is the gene product with an activity as a “At1g19800-protein”, preferably it is the molecule of section (a) or (b) of this paragraph.

In particular, it was observed that in plants, especially in Arabidopsis thaliana, increasing or generating the activity of a gene product non-targeted with the activity of a “At1g19800-protein”, preferably being encoded by a gene comprising the nucleic acid sequence SEQ ID NO. 17637, preferably the coding region thereof, conferred the production of or the increase in linoleic acid compared with the wild type control.

The nucleic acid sequence of At1g68320 from Arabidopsis thaliana, e.g. as shown in the respective line in column 5 of Table I, application no. 8, has been published in F. And the activity of the gene product thereof is the activity of transcription factor.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said At1g68320, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said At1g68320, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said At1g68320, or a functional equivalent or a homolog thereof as depicted in column 8 of Table II, application no. 8, preferably a homolog or functional equivalent as depicted in column 8 of Table II B, application no. 8, and being depicted in the same respective line as said At1g68320, and preferably the activity is increased non-targeted.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 1061, preferably the coding region thereof, conferred the production of or the increase in linoleic acid compared with the wild type control.

The nucleic acid sequence of At2g36580 from Arabidopsis thaliana, e.g. as shown in the respective line in column 5 of Table I, application no. 8, has been published in F. And the activity of the gene product thereof is the activity of pyruvate kinase.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “pyruvate kinase”, especially from Arabidopsis thaliana or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said At2g36580, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said At2g36580, and preferably the activity is increased plastidic, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said At2g36580, or a functional equivalent or a homolog thereof as depicted in column 8 of Table II, application no. 8, preferably a homolog or functional equivalent as depicted in column 8 of Table II B, application no. 8, and being depicted in the same respective line as said At2g36580, and preferably the activity is increased plastidic.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 113714, preferably the coding region thereof, conferred the production of or the increase in linoleic acid compared with the wild type control.

The nucleic acid sequence of At2g45420 from Arabidopsis thaliana, e.g. as shown in the respective line in column 5 of Table I, application no. 8, has been published in F. And the activity of the gene product thereof is the activity of At2g45420-protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “At2g45420-protein”, especially from Arabidopsis thaliana or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said At2g45420, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said At2g45420, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said At2g45420, or a functional equivalent or a homolog thereof as depicted in column 8 of Table II, application no. 8, preferably a homolog or functional equivalent as depicted in column 8 of Table II B, application no. 8, and being depicted in the same respective line as said At2g45420, and preferably the activity is increased non-targeted.

Accordingly, in one embodiment, the molecule which activity is to be increased in the process of the invention is the gene product with an activity as a “At2g45420-protein”, preferably it is the molecule of section (a) or (b) of this paragraph.

In particular, it was observed that in plants, especially in Arabidopsis thaliana, increasing or generating the activity of a gene product non-targeted with the activity of a “At2g45420-protein”, preferably being encoded by a gene comprising the nucleic acid sequence SEQ ID NO. 1623, preferably the coding region thereof, conferred the production of or the increase in linoleic acid compared with the wild type control.

The nucleic acid sequence of At3g02990 from Arabidopsis thaliana, e.g. as shown in the respective line in column 5 of Table I, application no. 8, has been published in F. And the activity of the gene product thereof is the activity of heat shock transcription factor.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said At3g02990, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said At3g02990, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said At3g02990, or a functional equivalent or a homolog thereof as depicted in column 8 of Table II, application no. 8, preferably a homolog or functional equivalent as depicted in column 8 of Table II B, application no. 8, and being depicted in the same respective line as said At3g02990, and preferably the activity is increased non-targeted.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 68657, preferably the coding region thereof, conferred the production of or the increase in linoleic acid compared with the wild type control.

The nucleic acid sequence of At3g20910 from Arabidopsis thaliana, e.g. as shown in the respective line in column 5 of Table I, application no. 8, has been published in F. And the activity of the gene product thereof is the activity of CCAAT-binding transcription factor.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “CCAAT-binding transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said At3g20910, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said At3g20910, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said At3g20910, or a functional equivalent or a homolog thereof as depicted in column 8 of Table II, application no. 8, preferably a homolog or functional equivalent as depicted in column 8 of Table II B, application no. 8, and being depicted in the same respective line as said At3g20910, and preferably the activity is increased non-targeted.

Accordingly, in one embodiment, the molecule which activity is to be increased in the process of the invention is the gene product with an activity as a “CCAAT-binding transcription factor”, preferably it is the molecule of section (a) or (b) of this paragraph.

In particular, it was observed that in plants, especially in Arabidopsis thaliana, increasing or generating the activity of a gene product non-targeted with the activity of a “CCAAT-binding transcription factor”, preferably being encoded by a gene comprising the nucleic acid sequence SEQ ID NO. 22921, preferably the coding region thereof, conferred the production of or the increase in linoleic acid compared with the wild type control.

The nucleic acid sequence of At3g62950 from Arabidopsis thaliana, e.g. as shown in the respective line in column 5 of Table I, application no. 8, has been published in F. And the activity of the gene product thereof is the activity of glutaredoxin.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said At3g62950, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said At3g62950, or a functional equivalent or a homolog thereof as depicted in column 8 of Table II, application no. 8, preferably a homolog or functional equivalent as depicted in column 8 of Table II B, application no. 8, and being depicted in the same respective line as said At3g62950, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in linoleic acid compared with the wild type control.

The nucleic acid sequence of At4g09960 from Arabidopsis thaliana, e.g. as shown in the respective line in column 5 of Table I, application no. 8, has been published in F. And the activity of the gene product thereof is the activity of transcription factor.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said At4g09960, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said At4g09960, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said At4g09960, or a functional equivalent or a homolog thereof as depicted in column 8 of Table II, application no. 8, preferably a homolog or functional equivalent as depicted in column 8 of Table II B, application no. 8, and being depicted in the same respective line as said At4g09960, and preferably the activity is increased non-targeted.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 109717, preferably the coding region thereof, conferred the production of or the increase in linoleic acid compared with the wild type control.

The nucleic acid sequence of At4g15660 from Arabidopsis thaliana, e.g. as shown in the respective line in column 5 of Table I, application no. 8, has been published in F. And the activity of the gene product thereof is the activity of monothiol glutaredoxin.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said At4g15660, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said At4g15660, or a functional equivalent or a homolog thereof as depicted in column 8 of Table II, application no. 8, preferably a homolog or functional equivalent as depicted in column 8 of Table II B, application no. 8, and being depicted in the same respective line as said At4g15660, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in linoleic acid compared with the wild type control.

The nucleic acid sequence of At4g15670 from Arabidopsis thaliana, e.g. as shown in the respective line in column 5 of Table I, application no. 8, has been published in F. And the activity of the gene product thereof is the activity of monothiol glutaredoxin.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said At4g15670, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said At4g15670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said At4g15670, or a functional equivalent or a homolog thereof as depicted in column 8 of Table II, application no. 8, preferably a homolog or functional equivalent as depicted in column 8 of Table II B, application no. 8, and being depicted in the same respective line as said At4g15670, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3279, preferably the coding region thereof, conferred the production of or the increase in linoleic acid compared with the wild type control.

The nucleic acid sequence of At4g26080 from Arabidopsis thaliana, e.g. as shown in the respective line in column 5 of Table I, application no. 8, has been published in F. And the activity of the gene product thereof is the activity of protein phosphatase.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said At4g26080, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said At4g26080, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said At4g26080, or a functional equivalent or a homolog thereof as depicted in column 8 of Table II, application no. 8, preferably a homolog or functional equivalent as depicted in column 8 of Table II B, application no. 8, and being depicted in the same respective line as said At4g26080, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 68849, preferably the coding region thereof, conferred the production of or the increase in linoleic acid compared with the wild type control.

The nucleic acid sequence of At4g33040 from Arabidopsis thaliana, e.g. as shown in the respective line in column 5 of Table I, application no. 8, has been published in F. And the activity of the gene product thereof is the activity of glutaredoxin.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said At4g33040, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said At4g33040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said At4g33040, or a functional equivalent or a homolog thereof as depicted in column 8 of Table II, application no. 8, preferably a homolog or functional equivalent as depicted in column 8 of Table II B, application no. 8, and being depicted in the same respective line as said At4g33040, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 4102, preferably the coding region thereof, conferred the production of or the increase in linoleic acid compared with the wild type control.

The nucleic acid sequence of At5g03720 from Arabidopsis thaliana, e.g. as shown in the respective line in column 5 of Table I, application no. 8, has been published in F. And the activity of the gene product thereof is the activity of heat shock transcription factor.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said At5g03720, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said At5g03720, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said At5g03720, or a functional equivalent or a homolog thereof as depicted in column 8 of Table II, application no. 8, preferably a homolog or functional equivalent as depicted in column 8 of Table II B, application no. 8, and being depicted in the same respective line as said At5g03720, and preferably the activity is increased non-targeted.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 24438, preferably the coding region thereof, conferred the production of or the increase in linoleic acid compared with the wild type control.

The nucleic acid sequence of At5g59220 from Arabidopsis thaliana, e.g. as shown in the respective line in column 5 of Table I, application no. 8, has been published in F. And the activity of the gene product thereof is the activity of protein phosphatase.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said At5g59220, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said At5g59220, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said At5g59220, or a functional equivalent or a homolog thereof as depicted in column 8 of Table II, application no. 8, preferably a homolog or functional equivalent as depicted in column 8 of Table II B, application no. 8, and being depicted in the same respective line as said At5g59220, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 25498, preferably the coding region thereof, conferred the production of or the increase in linoleic acid compared with the wild type control.

The nucleic acid sequence of AvinDRAFT_{—}1788 from Azotobacter vinelandii, e.g. as shown in the respective line in column 5 of Table I, application no. 8, has been published in D. And the activity of the gene product thereof is the activity of oxidoreductase.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “oxidoreductase”, especially from Azotobacter vinelandii or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said AvinDRAFT_{—}1788, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said AvinDRAFT_{—}1788, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said AvinDRAFT_{—}1788, or a functional equivalent or a homolog thereof as depicted in column 8 of Table II, application no. 8, preferably a homolog or functional equivalent as depicted in column 8 of Table II B, application no. 8, and being depicted in the same respective line as said AvinDRAFT_{—}1788, and preferably the activity is increased non-targeted.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 114083, preferably the coding region thereof, conferred the production of or the increase in linoleic acid compared with the wild type control.

The nucleic acid sequence of AvinDRAFT_{—}2010 from Azotobacter vinelandii, e.g. as shown in the respective line in column 5 of Table I, application no. 8, has been published in D. And the activity of the gene product thereof is the activity of transcriptional regulator protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “transcriptional regulator protein”, especially from Azotobacter vinelandii or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said AvinDRAFT_{—}2010, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said AvinDRAFT_{—}2010, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said AvinDRAFT_{—}2010, or a functional equivalent or a homolog thereof as depicted in column 8 of Table II, application no. 8, preferably a homolog or functional equivalent as depicted in column 8 of Table II B, application no. 8, and being depicted in the same respective line as said AvinDRAFT_{—}2010, and preferably the activity is increased non-targeted.

Accordingly, in one embodiment, the molecule which activity is to be increased in the process of the invention is the gene product with an activity as a “transcriptional regulator protein”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator protein”, preferably being encoded by a gene comprising the nucleic acid sequence SEQ ID NO. 114198, preferably the coding region thereof, conferred the production of or the increase in linoleic acid compared with the wild type control.

The nucleic acid sequence of AvinDRAFT_{—}2091 from Azotobacter vinelandii, e.g. as shown in the respective line in column 5 of Table I, application no. 8, has been published in D. And the activity of the gene product thereof is the activity of beta-hydroxylase.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “beta-hydroxylase”, especially from Azotobacter vinelandii or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said AvinDRAFT_{—}2091, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said AvinDRAFT_{—}2091, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said AvinDRAFT_{—}2091, or a functional equivalent or a homolog thereof as depicted in column 8 of Table II, application no. 8, preferably a homolog or functional equivalent as depicted in column 8 of Table II B, application no. 8, and being depicted in the same respective line as said AvinDRAFT_{—}2091, and preferably the activity is increased non-targeted.

Accordingly, in one embodiment, the molecule which activity is to be increased in the process of the invention is the gene product with an activity as a “beta-hydroxylase”, preferably it is the molecule of section (a) or (b) of this paragraph.

In particular, it was observed that in plants, especially in Arabidopsis thaliana, increasing or generating the activity of a gene product non-targeted with the activity of a “beta-hydroxylase”, preferably being encoded by a gene comprising the nucleic acid sequence SEQ ID NO. 6040, preferably the coding region thereof, conferred the production of or the increase in linoleic acid compared with the wild type control.

The nucleic acid sequence of AvinDRAFT_{—}3253 from Azotobacter vinelandii, e.g. as shown in the respective line in column 5 of Table I, application no. 8, has been published in D. And the activity of the gene product thereof is the activity of oxidoreductase.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “oxidoreductase”, especially from Azotobacter vinelandii or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said AvinDRAFT_{—}3253, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said AvinDRAFT_{—}3253, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said AvinDRAFT_{—}3253, or a functional equivalent or a homolog thereof as depicted in column 8 of Table II, application no. 8, preferably a homolog or functional equivalent as depicted in column 8 of Table II B, application no. 8, and being depicted in the same respective line as said AvinDRAFT_{—}3253, and preferably the activity is increased non-targeted.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 29500, preferably the coding region thereof, conferred the production of or the increase in linoleic acid compared with the wild type control.

The nucleic acid sequence of AvinDRAFT_{—}3577 from Azotobacter vinelandii, e.g. as shown in the respective line in column 5 of Table I, application no. 8, has been published in D. And the activity of the gene product thereof is the activity of ThiF family protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “ThiF family protein”, especially from Azotobacter vinelandii or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said AvinDRAFT_{—}3577, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said AvinDRAFT_{—}3577, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said AvinDRAFT_{—}3577, or a functional equivalent or a homolog thereof as depicted in column 8 of Table II, application no. 8, preferably a homolog or functional equivalent as depicted in column 8 of Table II B, application no. 8, and being depicted in the same respective line as said AvinDRAFT_{—}3577, and preferably the activity is increased non-targeted.

Accordingly, in one embodiment, the molecule which activity is to be increased in the process of the invention is the gene product with an activity as a “ThiF family protein”, preferably it is the molecule of section (a) or (b) of this paragraph.

In particular, it was observed that in plants, especially in Arabidopsis thaliana, increasing or generating the activity of a gene product non-targeted with the activity of a “ThiF family protein”, preferably being encoded by a gene comprising the nucleic acid sequence SEQ ID NO. 111155, preferably the coding region thereof, conferred the production of or the increase in linoleic acid compared with the wild type control.

The nucleic acid sequence of AvinDRAFT_{—}3629 from Azotobacter vinelandii, e.g. as shown in the respective line in column 5 of Table I, application no. 8, has been published in D. And the activity of the gene product thereof is the activity of recombinase A.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “recombinase A”, especially from Azotobacter vinelandii or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said AvinDRAFT_{—}3629, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said AvinDRAFT_{—}3629, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said AvinDRAFT_{—}3629, or a functional equivalent or a homolog thereof as depicted in column 8 of Table II, application no. 8, preferably a homolog or functional equivalent as depicted in column 8 of Table II B, application no. 8, and being depicted in the same respective line as said AvinDRAFT_{—}3629, and preferably the activity is increased non-targeted.

Accordingly, in one embodiment, the molecule which activity is to be increased in the process of the invention is the gene product with an activity as a “recombinase A”, preferably it is the molecule of section (a) or (b) of this paragraph.

In particular, it was observed that in plants, especially in Arabidopsis thaliana, increasing or generating the activity of a gene product non-targeted with the activity of a “recombinase A”, preferably being encoded by a gene comprising the nucleic acid sequence SEQ ID NO. 114231, preferably the coding region thereof, conferred the production of or the increase in linoleic acid compared with the wild type control.

The nucleic acid sequence of AvinDRAFT_{—}5103 from Azotobacter vinelandii, e.g. as shown in the respective line in column 5 of Table I, application no. 8, has been published in D. And the activity of the gene product thereof is the activity of hydrolase.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “hydrolase”, especially from Azotobacter vinelandii or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said AvinDRAFT_{—}5103, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said AvinDRAFT_{—}5103, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said AvinDRAFT_{—}5103, or a functional equivalent or a homolog thereof as depicted in column 8 of Table II, application no. 8, preferably a homolog or functional equivalent as depicted in column 8 of Table II B, application no. 8, and being depicted in the same respective line as said AvinDRAFT_{—}5103, and preferably the activity is increased non-targeted.

Accordingly, in one embodiment, the molecule which activity is to be increased in the process of the invention is the gene product with an activity as a “hydrolase”, preferably it is the molecule of section (a) or (b) of this paragraph.

In particular, it was observed that in plants, especially in Arabidopsis thaliana, increasing or generating the activity of a gene product non-targeted with the activity of a “hydrolase”, preferably being encoded by a gene comprising the nucleic acid sequence SEQ ID NO. 6510, preferably the coding region thereof, conferred the production of or the increase in linoleic acid compared with the wild type control.

The nucleic acid sequence of B0486 from Escherichia coli, e.g. as shown in the respective line in column 5 of Table I, application no. 8, has been published in B. And the activity of the gene product thereof is the activity of membrane transport protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “membrane transport protein”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B0486, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B0486, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B0486, or a functional equivalent or a homolog thereof as depicted in column 8 of Table II, application no. 8, preferably a homolog or functional equivalent as depicted in column 8 of Table II B, application no. 8, and being depicted in the same respective line as said B0486, and preferably the activity is increased non-targeted.

Accordingly, in one embodiment, the molecule which activity is to be increased in the process of the invention is the gene product with an activity as a “membrane transport protein”, preferably it is the molecule of section (a) or (b) of this paragraph.

In particular, it was observed that in plants, especially in Arabidopsis thaliana, increasing or generating the activity of a gene product non-targeted with the activity of a “membrane transport protein”, preferably being encoded by a gene comprising the nucleic acid sequence SEQ ID NO. 7686, preferably the coding region thereof, conferred the production of or the increase in linoleic acid compared with the wild type control.

The nucleic acid sequence of B0801 from Escherichia coli, e.g. as shown in the respective line in column 5 of Table I, application no. 8, has been published in B. And the activity of the gene product thereof is the activity of b0801-protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “b0801-protein”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B0801, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B0801, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B0801, or a functional equivalent or a homolog thereof as depicted in column 8 of Table II, application no. 8, preferably a homolog or functional equivalent as depicted in column 8 of Table II B, application no. 8, and being depicted in the same respective line as said B0801, and preferably the activity is increased non-targeted.

Accordingly, in one embodiment, the molecule which activity is to be increased in the process of the invention is the gene product with an activity as a “b0801-protein”, preferably it is the molecule of section (a) or (b) of this paragraph.

In particular, it was observed that in plants, especially in Arabidopsis thaliana, increasing or generating the activity of a gene product non-targeted with the activity of a “b0801-protein”, preferably being encoded by a gene comprising the nucleic acid sequence SEQ ID NO. 75807, preferably the coding region thereof, conferred the production of or the increase in linoleic acid compared with the wild type control.

The nucleic acid sequence of B1095 from Escherichia coli, e.g. as shown in the respective line in column 5 of Table I, application no. 8, has been published in B. And the activity of the gene product thereof is the activity of 3-oxoacyl-[acyl-carrier-protein] synthase.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “3-oxoacyl-[acyl-carrier-protein] synthase”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B1095, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B1095, and preferably the activity is increased plastidic, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B1095, or a functional equivalent or a homolog thereof as depicted in column 8 of Table II, application no. 8, preferably a homolog or functional equivalent as depicted in column 8 of Table II B, application no. 8, and being depicted in the same respective line as said B1095, and preferably the activity is increased plastidic.

Accordingly, in one embodiment, the molecule which activity is to be increased in the process of the invention is the gene product with an activity as a “3-oxoacyl-[acyl-carrier-protein] synthase”, preferably it is the molecule of section (a) or (b) of this paragraph.

In particular, it was observed that in plants, especially in Arabidopsis thaliana, increasing or generating the activity of a gene product plastidic with the activity of a “3-oxoacyl-[acyl-carrier-protein] synthase”, preferably being encoded by a gene comprising the nucleic acid sequence SEQ ID NO. 114552, preferably the coding region thereof, conferred the production of or the increase in linoleic acid compared with the wild type control.

The nucleic acid sequence of B1102 from Escherichia coli, e.g. as shown in the respective line in column 5 of Table I, application no. 8, has been published in B. And the activity of the gene product thereof is the activity of outer membrane receptor protein precursor.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “outer membrane receptor protein precursor”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B1102, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B1102, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B1102, or a functional equivalent or a homolog thereof as depicted in column 8 of Table II, application no. 8, preferably a homolog or functional equivalent as depicted in column 8 of Table II B, application no. 8, and being depicted in the same respective line as said B1102, and preferably the activity is increased non-targeted.

Accordingly, in one embodiment, the molecule which activity is to be increased in the process of the invention is the gene product with an activity as a “outer membrane receptor protein precursor”, preferably it is the molecule of section (a) or (b) of this paragraph.

In particular, it was observed that in plants, especially in Arabidopsis thaliana, increasing or generating the activity of a gene product non-targeted with the activity of a “outer membrane receptor protein precursor”, preferably being encoded by a gene comprising the nucleic acid sequence SEQ ID NO. 114979, preferably the coding region thereof, conferred the production of or the increase in linoleic acid compared with the wild type control.

The nucleic acid sequence of B1330 from Escherichia coli, e.g. as shown in the respective line in column 5 of Table I, application no. 8, has been published in B. And the activity of the gene product thereof is the activity of b1330-protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “b1330-protein”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B1330, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B1330, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B1330, or a functional equivalent or a homolog thereof as depicted in column 8 of Table II, application no. 8, preferably a homolog or functional equivalent as depicted in column 8 of Table II B, application no. 8, and being depicted in the same respective line as said B1330, and preferably the activity is increased non-targeted.

Accordingly, in one embodiment, the molecule which activity is to be increased in the process of the invention is the gene product with an activity as a “b1330-protein”, preferably it is the molecule of section (a) or (b) of this paragraph.

In particular, it was observed that in plants, especially in Arabidopsis thaliana, increasing or generating the activity of a gene product non-targeted with the activity of a “b1330-protein”, preferably being encoded by a gene comprising the nucleic acid sequence SEQ ID NO. 38226, preferably the coding region thereof, conferred the production of or the increase in linoleic acid compared with the wild type control.

The nucleic acid sequence of B1431 from Escherichia coli, e.g. as shown in the respective line in column 5 of Table I, application no. 8, has been published in B. And the activity of the gene product thereof is the activity of lipoprotein precursor.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “lipoprotein precursor”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B1431, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B1431, and preferably the activity is increased plastidic, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B1431, or a functional equivalent or a homolog thereof as depicted in column 8 of Table II, application no. 8, preferably a homolog or functional equivalent as depicted in column 8 of Table II B, application no. 8, and being depicted in the same respective line as said B1431, and preferably the activity is increased plastidic.

Accordingly, in one embodiment, the molecule which activity is to be increased in the process of the invention is the gene product with an activity as a “lipoprotein precursor”, preferably it is the molecule of section (a) or (b) of this paragraph.

In particular, it was observed that in plants, especially in Arabidopsis thaliana, increasing or generating the activity of a gene product plastidic with the activity of a “lipoprotein precursor”, preferably being encoded by a gene comprising the nucleic acid sequence SEQ ID NO. 38266, preferably the coding region thereof, conferred the production of or the increase in linoleic acid compared with the wild type control.

The nucleic acid sequence of B1470 from Escherichia coli, e.g. as shown in the respective line in column 5 of Table I, application no. 8, has been published in B. And the activity of the gene product thereof is the activity of b1470-protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “b1470-protein”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B1470, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B1470, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B1470, or a functional equivalent or a homolog thereof as depicted in column 8 of Table II, application no. 8, preferably a homolog or functional equivalent as depicted in column 8 of Table II B, application no. 8, and being depicted in the same respective line as said B1470, and preferably the activity is increased non-targeted.

Accordingly, in one embodiment, the molecule which activity is to be increased in the process of the invention is the gene product with an activity as a “b1470-protein”, preferably it is the molecule of section (a) or (b) of this paragraph.

In particular, it was observed that in plants, especially in Arabidopsis thaliana, increasing or generating the activity of a gene product non-targeted with the activity of a “b1470-protein”, preferably being encoded by a gene comprising the nucleic acid sequence SEQ ID NO. 115056, preferably the coding region thereof, conferred the production of or the increase in linoleic acid compared with the wild type control.

The nucleic acid sequence of B1589 from Escherichia coli, e.g. as shown in the respective line in column 5 of Table I, application no. 8, has been published in B. And the activity of the gene product thereof is the activity of Fe—S subunit of oxidoreductase.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “Fe—S subunit of oxidoreductase”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B1589, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B1589, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B1589, or a functional equivalent or a homolog thereof as depicted in column 8 of Table II, application no. 8, preferably a homolog or functional equivalent as depicted in column 8 of Table II B, application no. 8, and being depicted in the same respective line as said B1589, and preferably the activity is increased non-targeted.

Accordingly, in one embodiment, the molecule which activity is to be increased in the process of the invention is the gene product with an activity as a “Fe—S subunit of oxidoreductase”, preferably it is the molecule of section (a) or (b) of this paragraph.

In particular, it was observed that in plants, especially in Arabidopsis thaliana, increasing or generating the activity of a gene product non-targeted with the activity of a “Fe—S subunit of oxidoreductase”, preferably being encoded by a gene comprising the nucleic acid sequence SEQ ID NO. 78154, preferably the coding region thereof, conferred the production of or the increase in linoleic acid compared with the wild type control.

The nucleic acid sequence of B1597 from Escherichia coli, e.g. as shown in the respective line in column 5 of Table I, application no. 8, has been published in B. And the activity of the gene product thereof is the activity of acid shock protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “acid shock protein”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B1597, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B1597, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B1597, or a functional equivalent or a homolog thereof as depicted in column 8 of Table II, application no. 8, preferably a homolog or functional equivalent as depicted in column 8 of Table II B, application no. 8, and being depicted in the same respective line as said B1597, and preferably the activity is increased non-targeted.

Accordingly, in one embodiment, the molecule which activity is to be increased in the process of the invention is the gene product with an activity as a “acid shock protein”, preferably it is the molecule of section (a) or (b) of this paragraph.

In particular, it was observed that in plants, especially in Arabidopsis thaliana, increasing or generating the activity of a gene product non-targeted with the activity of a “acid shock protein”, preferably being encoded by a gene comprising the nucleic acid sequence SEQ ID NO. 38300, preferably the coding region thereof, conferred the production of or the increase in linoleic acid compared with the wild type control.

The nucleic acid sequence of B1611 from Escherichia coli, e.g. as shown in the respective line in column 5 of Table I, application no. 8, has been published in B. And the activity of the gene product thereof is the activity of fumarate hydratase.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “fumarate hydratase”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B1611, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B1611, and preferably the activity is increased plastidic, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B1611, or a functional equivalent or a homolog thereof as depicted in column 8 of Table II, application no. 8, preferably a homolog or functional equivalent as depicted in column 8 of Table II B, application no. 8, and being depicted in the same respective line as said B1611, and preferably the activity is increased plastidic.

Accordingly, in one embodiment, the molecule which activity is to be increased in the process of the invention is the gene product with an activity as a “fumarate hydratase”, preferably it is the molecule of section (a) or (b) of this paragraph.

In particular, it was observed that in plants, especially in Arabidopsis thaliana, increasing or generating the activity of a gene product plastidic with the activity of a “fumarate hydratase”, preferably being encoded by a gene comprising the nucleic acid sequence SEQ ID NO. 115064, preferably the coding region thereof, conferred the production of or the increase in linoleic acid compared with the wild type control.

The nucleic acid sequence of B1627 from Escherichia coli, e.g. as shown in the respective line in column 5 of Table I, application no. 8, has been published in B. And the activity of the gene product thereof is the activity of electron transport complex protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “electron transport complex protein”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B1627, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B1627, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B1627, or a functional equivalent or a homolog thereof as depicted in column 8 of Table II, application no. 8, preferably a homolog or functional equivalent as depicted in column 8 of Table II B, application no. 8, and being depicted in the same respective line as said B1627, and preferably the activity is increased non-targeted.

Accordingly, in one embodiment, the molecule which activity is to be increased in the process of the invention is the gene product with an activity as a “electron transport complex protein”, preferably it is the molecule of section (a) or (b) of this paragraph.

In particular, it was observed that in plants, especially in Arabidopsis thaliana, increasing or generating the activity of a gene product non-targeted with the activity of a “electron transport complex protein”, preferably being encoded by a gene comprising the nucleic acid sequence SEQ ID NO. 38345, preferably the coding region thereof, conferred the production of or the increase in linoleic acid compared with the wild type control.

The nucleic acid sequence of B1670 from Escherichia coli, e.g. as shown in the respective line in column 5 of Table I, application no. 8, has been published in B. And the activity of the gene product thereof is the activity of b1670-protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “b1670-protein”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B1670, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B1670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B1670, or a functional equivalent or a homolog thereof as depicted in column 8 of Table II, application no. 8, preferably a homolog or functional equivalent as depicted in column 8 of Table II B, application no. 8, and being depicted in the same respective line as said B1670, and preferably the activity is increased non-targeted.

Accordingly, in one embodiment, the molecule which activity is to be increased in the process of the invention is the gene product with an activity as a “b1670-protein”, preferably it is the molecule of section (a) or (b) of this paragraph.

In particular, it was observed that in plants, especially in Arabidopsis thaliana, increasing or generating the activity of a gene product non-targeted with the activity of a “b1670-protein”, preferably being encoded by a gene comprising the nucleic acid sequence SEQ ID NO. 78753, preferably the coding region thereof, conferred the production of or the increase in linoleic acid compared with the wild type control.

The nucleic acid sequence of B1837 from Escherichia coli, e.g. as shown in the respective line in column 5 of Table I, application no. 8, has been published in B. And the activity of the gene product thereof is the activity of b1837-protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “b1837-protein”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B1837, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B1837, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B1837, or a functional equivalent or a homolog thereof as depicted in column 8 of Table II, application no. 8, preferably a homolog or functional equivalent as depicted in column 8 of Table II B, application no. 8, and being depicted in the same respective line as said B1837, and preferably the activity is increased non-targeted.

Accordingly, in one embodiment, the molecule which activity is to be increased in the process of the invention is the gene product with an activity as a “b1837-protein”, preferably it is the molecule of section (a) or (b) of this paragraph.

In particular, it was observed that in plants, especially in Arabidopsis thaliana, increasing or generating the activity of a gene product non-targeted with the activity of a “b1837-protein”, preferably being encoded by a gene comprising the nucleic acid sequence SEQ ID NO. 78953, preferably the coding region thereof, conferred the production of or the increase in linoleic acid compared with the wild type control.

The nucleic acid sequence of B2032 from Escherichia coli, e.g. as shown in the respective line in column 5 of Table I, application no. 8, has been published in B. And the activity of the gene product thereof is the activity of b2032-protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “b2032-protein”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B2032, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B2032, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B2032, or a functional equivalent or a homolog thereof as depicted in column 8 of Table II, application no. 8, preferably a homolog or functional equivalent as depicted in column 8 of Table II B, application no. 8, and being depicted in the same respective line as said B2032, and preferably the activity is increased non-targeted.

Accordingly, in one embodiment, the molecule which activity is to be increased in the process of the invention is the gene product with an activity as a “b2032-protein”, preferably it is the molecule of section (a) or (b) of this paragraph.

In particular, it was observed that in plants, especially in Arabidopsis thaliana, increasing or generating the activity of a gene product non-targeted with the activity of a “b2032-protein”, preferably being encoded by a gene comprising the nucleic acid sequence SEQ ID NO. 8920, preferably the coding region thereof, conferred the production of or the increase in linoleic acid compared with the wild type control.

The nucleic acid sequence of B2043 from Escherichia coli, e.g. as shown in the respective line in column 5 of Table I, application no. 8, has been published in B. And the activity of the gene product thereof is the activity of colanic acid biosynthesis protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “colanic acid biosynthesis protein”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B2043, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B2043, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B2043, or a functional equivalent or a homolog thereof as depicted in column 8 of Table II, application no. 8, preferably a homolog or functional equivalent as depicted in column 8 of Table II B, application no. 8, and being depicted in the same respective line as said B2043, and preferably the activity is increased non-targeted.

Accordingly, in one embodiment, the molecule which activity is to be increased in the process of the invention is the gene product with an activity as a “colanic acid biosynthesis protein”, preferably it is the molecule of section (a) or (b) of this paragraph.

In particular, it was observed that in plants, especially in Arabidopsis thaliana, increasing or generating the activity of a gene product non-targeted with the activity of a “colanic acid biosynthesis protein”, preferably being encoded by a gene comprising the nucleic acid sequence SEQ ID NO. 112193, preferably the coding region thereof, conferred the production of or the increase in linoleic acid compared with the wild type control.

The nucleic acid sequence of B2099 from Escherichia coli, e.g. as shown in the respective line in column 5 of Table I, application no. 8, has been published in B. And the activity of the gene product thereof is the activity of b2099-protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “b2099-protein”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B2099, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B2099, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B2099, or a functional equivalent or a homolog thereof as depicted in column 8 of Table II, application no. 8, preferably a homolog or functional equivalent as depicted in column 8 of Table II B, application no. 8, and being depicted in the same respective line as said B2099, and preferably the activity is increased non-targeted.

Accordingly, in one embodiment, the molecule which activity is to be increased in the process of the invention is the gene product with an activity as a “b2099-protein”, preferably it is the molecule of section (a) or (b) of this paragraph.

In particular, it was observed that in plants, especially in Arabidopsis thaliana, increasing or generating the activity of a gene product non-targeted with the activity of a “b2099-protein”, preferably being encoded by a gene comprising the nucleic acid sequence SEQ ID NO. 115595, preferably the coding region thereof, conferred the production of or the increase in linoleic acid compared with the wild type control.

The nucleic acid sequence of B2107 from Escherichia coli, e.g. as shown in the respective line in column 5 of Table I, application no. 8, has been published in B. And the activity of the gene product thereof is the activity of b2107-protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “b2107-protein”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B2107, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B2107, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B2107, or a functional equivalent or a homolog thereof as depicted in column 8 of Table II, application no. 8, preferably a homolog or functional equivalent as depicted in column 8 of Table II B, application no. 8, and being depicted in the same respective line as said B2107, and preferably the activity is increased non-targeted.

Accordingly, in one embodiment, the molecule which activity is to be increased in the process of the invention is the gene product with an activity as a “b2107-protein”, preferably it is the molecule of section (a) or (b) of this paragraph.

In particular, it was observed that in plants, especially in Arabidopsis thaliana, increasing or generating the activity of a gene product non-targeted with the activity of a “b2107-protein”, preferably being encoded by a gene comprising the nucleic acid sequence SEQ ID NO. 39002, preferably the coding region thereof, conferred the production of or the increase in linoleic acid compared with the wild type control.

The nucleic acid sequence of B2178 from Escherichia coli, e.g. as shown in the respective line in column 5 of Table I, application no. 8, has been published in B. And the activity of the gene product thereof is the activity of ABC transporter permease protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “ABC transporter permease protein”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B2178, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B2178, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B2178, or a functional equivalent or a homolog thereof as depicted in column 8 of Table II, application no. 8, preferably a homolog or functional equivalent as depicted in column 8 of Table II B, application no. 8, and being depicted in the same respective line as said B2178, and preferably the activity is increased non-targeted.

Accordingly, in one embodiment, the molecule which activity is to be increased in the process of the invention is the gene product with an activity as a “ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “ABC transporter permease protein”, preferably being encoded by a gene comprising the nucleic acid sequence SEQ ID NO. 39040, preferably the coding region thereof, conferred the production of or the increase in linoleic acid compared with the wild type control.

The nucleic acid sequence of B2399 from Escherichia coli, e.g. as shown in the respective line in column 5 of Table I, application no. 8, has been published in B. And the activity of the gene product thereof is the activity of b2399-protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “b2399-protein”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B2399, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B2399, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B2399, or a functional equivalent or a homolog thereof as depicted in column 8 of Table II, application no. 8, preferably a homolog or functional equivalent as depicted in column 8 of Table II B, application no. 8, and being depicted in the same respective line as said B2399, and preferably the activity is increased non-targeted.

Accordingly, in one embodiment, the molecule which activity is to be increased in the process of the invention is the gene product with an activity as a “b2399-protein”, preferably it is the molecule of section (a) or (b) of this paragraph.

In particular, it was observed that in plants, especially in Arabidopsis thaliana, increasing or generating the activity of a gene product non-targeted with the activity of a “b2399-protein”, preferably being encoded by a gene comprising the nucleic acid sequence SEQ ID NO. 39237, preferably the coding region thereof, conferred the production of or the increase in linoleic acid compared with the wild type control.

The nucleic acid sequence of B2439 from Escherichia coli, e.g. as shown in the respective line in column 5 of Table I, application no. 8, has been published in B. And the activity of the gene product thereof is the activity of ethanolamine utilization protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “ethanolamine utilization protein”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B2439, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B2439, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B2439, or a functional equivalent or a homolog thereof as depicted in column 8 of Table II, application no. 8, preferably a homolog or functional equivalent as depicted in column 8 of Table II B, application no. 8, and being depicted in the same respective line as said B2439, and preferably the activity is increased non-targeted.

Accordingly, in one embodiment, the molecule which activity is to be increased in the process of the invention is the gene product with an activity as a “ethanolamine utilization protein”, preferably it is the molecule of section (a) or (b) of this paragraph.

In particular, it was observed that in plants, especially in Arabidopsis thaliana, increasing or generating the activity of a gene product non-targeted with the activity of a “ethanolamine utilization protein”, preferably being encoded by a gene comprising the nucleic acid sequence SEQ ID NO. 115628, preferably the coding region thereof, conferred the production of or the increase in linoleic acid compared with the wild type control.

The nucleic acid sequence of B2474 from Escherichia coli, e.g. as shown in the respective line in column 5 of Table I, application no. 8, has been published in B. And the activity of the gene product thereof is the activity of b2474-protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “b2474-protein”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B2474, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B2474, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B2474, or a functional equivalent or a homolog thereof as depicted in column 8 of Table II, application no. 8, preferably a homolog or functional equivalent as depicted in column 8 of Table II B, application no. 8, and being depicted in the same respective line as said B2474, and preferably the activity is increased non-targeted.

Accordingly, in one embodiment, the molecule which activity is to be increased in the process of the invention is the gene product with an activity as a “b2474-protein”, preferably it is the molecule of section (a) or (b) of this paragraph.

In particular, it was observed that in plants, especially in Arabidopsis thaliana, increasing or generating the activity of a gene product non-targeted with the activity of a “b2474-protein”, preferably being encoded by a gene comprising the nucleic acid sequence SEQ ID NO. 40329, preferably the coding region thereof, conferred the production of or the increase in linoleic acid compared with the wild type control.

The nucleic acid sequence of B2613 from Escherichia coli, e.g. as shown in the respective line in column 5 of Table I, application no. 8, has been published in B. And the activity of the gene product thereof is the activity of b2613-protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “b2613-protein”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B2613, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B2613, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B2613, or a functional equivalent or a homolog thereof as depicted in column 8 of Table II, application no. 8, preferably a homolog or functional equivalent as depicted in column 8 of Table II B, application no. 8, and being depicted in the same respective line as said B2613, and preferably the activity is increased non-targeted.

Accordingly, in one embodiment, the molecule which activity is to be increased in the process of the invention is the gene product with an activity as a “b2613-protein”, preferably it is the molecule of section (a) or (b) of this paragraph.

In particular, it was observed that in plants, especially in Arabidopsis thaliana, increasing or generating the activity of a gene product non-targeted with the activity of a “b2613-protein”, preferably being encoded by a gene comprising the nucleic acid sequence SEQ ID NO. 40665, preferably the coding region thereof, conferred the production of or the increase in linoleic acid compared with the wild type control.

The nucleic acid sequence of B2682 from Escherichia coli, e.g. as shown in the respective line in column 5 of Table I, application no. 8, has been published in B. And the activity of the gene product thereof is the activity of b2682-protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “b2682-protein”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B2682, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B2682, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B2682, or a functional equivalent or a homolog thereof as depicted in column 8 of Table II, application no. 8, preferably a homolog or functional equivalent as depicted in column 8 of Table II B, application no. 8, and being depicted in the same respective line as said B2682, and preferably the activity is increased non-targeted.

Accordingly, in one embodiment, the molecule which activity is to be increased in the process of the invention is the gene product with an activity as a “b2682-protein”, preferably it is the molecule of section (a) or (b) of this paragraph.

In particular, it was observed that in plants, especially in Arabidopsis thaliana, increasing or generating the activity of a gene product non-targeted with the activity of a “b2682-protein”, preferably being encoded by a gene comprising the nucleic acid sequence SEQ ID NO. 115655, preferably the coding region thereof, conferred the production of or the increase in linoleic acid compared with the wild type control.

The nucleic acid sequence of B2790 from Escherichia coli, e.g. as shown in the respective line in column 5 of Table I, application no. 8, has been published in B. And the activity of the gene product thereof is the activity of flavodoxin.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “flavodoxin”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B2790, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B2790, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B2790, or a functional equivalent or a homolog thereof as depicted in column 8 of Table II, application no. 8, preferably a homolog or functional equivalent as depicted in column 8 of Table II B, application no. 8, and being depicted in the same respective line as said B2790, and preferably the activity is increased non-targeted.

Accordingly, in one embodiment, the molecule which activity is to be increased in the process of the invention is the gene product with an activity as a “flavodoxin”, preferably it is the molecule of section (a) or (b) of this paragraph.

In particular, it was observed that in plants, especially in Arabidopsis thaliana, increasing or generating the activity of a gene product non-targeted with the activity of a “flavodoxin”, preferably being encoded by a gene comprising the nucleic acid sequence SEQ ID NO. 115719, preferably the coding region thereof, conferred the production of or the increase in linoleic acid compared with the wild type control.

The nucleic acid sequence of B2836 from Escherichia coli, e.g. as shown in the respective line in column 5 of Table I, application no. 8, has been published in B. And the activity of the gene product thereof is the activity of AAS bifunctional protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “AAS bifunctional protein”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B2836, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B2836, and preferably the activity is increased plastidic, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B2836, or a functional equivalent or a homolog thereof as depicted in column 8 of Table II, application no. 8, preferably a homolog or functional equivalent as depicted in column 8 of Table II B, application no. 8, and being depicted in the same respective line as said B2836, and preferably the activity is increased plastidic.

Accordingly, in one embodiment, the molecule which activity is to be increased in the process of the invention is the gene product with an activity as a “AAS bifunctional protein”, preferably it is the molecule of section (a) or (b) of this paragraph.

In particular, it was observed that in plants, especially in Arabidopsis thaliana, increasing or generating the activity of a gene product plastidic with the activity of a “AAS bifunctional protein”, preferably being encoded by a gene comprising the nucleic acid sequence SEQ ID NO. 115791, preferably the coding region thereof, conferred the production of or the increase in linoleic acid compared with the wild type control.

The nucleic acid sequence of B2849 from Escherichia coli, e.g. as shown in the respective line in column 5 of Table I, application no. 8, has been published in B. And the activity of the gene product thereof is the activity of b2849-protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “b2849-protein”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B2849, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B2849, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B2849, or a functional equivalent or a homolog thereof as depicted in column 8 of Table II, application no. 8, preferably a homolog or functional equivalent as depicted in column 8 of Table II B, application no. 8, and being depicted in the same respective line as said B2849, and preferably the activity is increased non-targeted.

Accordingly, in one embodiment, the molecule which activity is to be increased in the process of the invention is the gene product with an activity as a “b2849-protein”, preferably it is the molecule of section (a) or (b) of this paragraph.

In particular, it was observed that in plants, especially in Arabidopsis thaliana, increasing or generating the activity of a gene product non-targeted with the activity of a “b2849-protein”, preferably being encoded by a gene comprising the nucleic acid sequence SEQ ID NO. 81980, preferably the coding region thereof, conferred the production of or the increase in linoleic acid compared with the wild type control.

The nucleic acid sequence of B2866 from Escherichia coli, e.g. as shown in the respective line in column 5 of Table I, application no. 8, has been published in B. And the activity of the gene product thereof is the activity of xanthine dehydrogenase.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “xanthine dehydrogenase”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B2866, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B2866, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B2866, or a functional equivalent or a homolog thereof as depicted in column 8 of Table II, application no. 8, preferably a homolog or functional equivalent as depicted in column 8 of Table II B, application no. 8, and being depicted in the same respective line as said B2866, and preferably the activity is increased non-targeted.

Accordingly, in one embodiment, the molecule which activity is to be increased in the process of the invention is the gene product with an activity as a “xanthine dehydrogenase”, preferably it is the molecule of section (a) or (b) of this paragraph.

In particular, it was observed that in plants, especially in Arabidopsis thaliana, increasing or generating the activity of a gene product non-targeted with the activity of a “xanthine dehydrogenase”, preferably being encoded by a gene comprising the nucleic acid sequence SEQ ID NO. 115864, preferably the coding region thereof, conferred the production of or the increase in linoleic acid compared with the wild type control.

The nucleic acid sequence of B2909 from Escherichia coli, e.g. as shown in the respective line in column 5 of Table I, application no. 8, has been published in B. And the activity of the gene product thereof is the activity of b2909-protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “b2909-protein”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B2909, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B2909, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B2909, or a functional equivalent or a homolog thereof as depicted in column 8 of Table II, application no. 8, preferably a homolog or functional equivalent as depicted in column 8 of Table II B, application no. 8, and being depicted in the same respective line as said B2909, and preferably the activity is increased non-targeted.

Accordingly, in one embodiment, the molecule which activity is to be increased in the process of the invention is the gene product with an activity as a “b2909-protein”, preferably it is the molecule of section (a) or (b) of this paragraph.

In particular, it was observed that in plants, especially in Arabidopsis thaliana, increasing or generating the activity of a gene product non-targeted with the activity of a “b2909-protein”, preferably being encoded by a gene comprising the nucleic acid sequence SEQ ID NO. 41006, preferably the coding region thereof, conferred the production of or the increase in linoleic acid compared with the wild type control.

The nucleic acid sequence of B3379 from Escherichia coli, e.g. as shown in the respective line in column 5 of Table I, application no. 8, has been published in B. And the activity of the gene product thereof is the activity of phosphotriesterase.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “phosphotriesterase”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B3379, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B3379, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B3379, or a functional equivalent or a homolog thereof as depicted in column 8 of Table II, application no. 8, preferably a homolog or functional equivalent as depicted in column 8 of Table II B, application no. 8, and being depicted in the same respective line as said B3379, and preferably the activity is increased non-targeted.

Accordingly, in one embodiment, the molecule which activity is to be increased in the process of the invention is the gene product with an activity as a “phosphotriesterase”, preferably it is the molecule of section (a) or (b) of this paragraph.

In particular, it was observed that in plants, especially in Arabidopsis thaliana, increasing or generating the activity of a gene product non-targeted with the activity of a “phosphotriesterase”, preferably being encoded by a gene comprising the nucleic acid sequence SEQ ID NO. 116110, preferably the coding region thereof, conferred the production of or the increase in linoleic acid compared with the wild type control.

The nucleic acid sequence of B3451 from Escherichia coli, e.g. as shown in the respective line in column 5 of Table I, application no. 8, has been published in B. And the activity of the gene product thereof is the activity of sn-glycerol-3-phosphate transport system permease protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “sn-glycerol-3-phosphate transport system permease protein”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B3451, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B3451, and preferably the activity is increased plastidic, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B3451, or a functional equivalent or a homolog thereof as depicted in column 8 of Table II, application no. 8, preferably a homolog or functional equivalent as depicted in column 8 of Table II B, application no. 8, and being depicted in the same respective line as said B3451, and preferably the activity is increased plastidic.

Accordingly, in one embodiment, the molecule which activity is to be increased in the process of the invention is the gene product with an activity as a “sn-glycerol-3-phosphate transport system permease protein”, preferably it is the molecule of section (a) or (b) of this paragraph.

In particular, it was observed that in plants, especially in Arabidopsis thaliana, increasing or generating the activity of a gene product plastidic with the activity of a “sn-glycerol-3-phosphate transport system permease protein”, preferably being encoded by a gene comprising the nucleic acid sequence SEQ ID NO. 116172, preferably the coding region thereof, conferred the production of or the increase in linoleic acid compared with the wild type control.

The nucleic acid sequence of B3823 from Escherichia coli, e.g. as shown in the respective line in column 5 of Table I, application no. 8, has been published in B. And the activity of the gene product thereof is the activity of threonine efflux protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “threonine efflux protein”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B3823, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B3823, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B3823, or a functional equivalent or a homolog thereof as depicted in column 8 of Table II, application no. 8, preferably a homolog or functional equivalent as depicted in column 8 of Table II B, application no. 8, and being depicted in the same respective line as said B3823, and preferably the activity is increased non-targeted.

Accordingly, in one embodiment, the molecule which activity is to be increased in the process of the invention is the gene product with an activity as a “threonine efflux protein”, preferably it is the molecule of section (a) or (b) of this paragraph.

In particular, it was observed that in plants, especially in Arabidopsis thaliana, increasing or generating the activity of a gene product non-targeted with the activity of a “threonine efflux protein”, preferably being encoded by a gene comprising the nucleic acid sequence SEQ ID NO. 10726, preferably the coding region thereof, conferred the production of or the increase in linoleic acid compared with the wild type control.

The nucleic acid sequence of B3845 from Escherichia coli, e.g. as shown in the respective line in column 5 of Table I, application no. 8, has been published in B. And the activity of the gene product thereof is the activity of 3-ketoacyl-CoA thiolase.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “3-ketoacyl-CoA thiolase”, especially from Escherichia coli or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said B3845, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said B3845, and preferably the activity is increased plastidic, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said B3845, or a functional equivalent or a homolog thereof as depicted in column 8 of Table II, application no. 8, preferably a homolog or functional equivalent as depicted in column 8 of Table II B, application no. 8, and being depicted in the same respective line as said B3845, and preferably the activity is increased plastidic.

Accordingly, in one embodiment, the molecule which activity is to be increased in the process of the invention is the gene product with an activity as a “3-ketoacyl-CoA thiolase”, preferably it is the molecule of section (a) or (b) of this paragraph.

In particular, it was observed that in plants, especially in Arabidopsis thaliana, increasing or generating the activity of a gene product plastidic with the activity of a “3-ketoacyl-CoA thiolase”, preferably being encoded by a gene comprising the nucleic acid sequence SEQ ID NO. 100675, preferably the coding region thereof, conferred the production of or the increase in linoleic acid compared with the wild type control.

The nucleic acid sequence of GM02LC38418 from Glycine max, e.g. as shown in the respective line in column 5 of Table I, application no. 8, is unpublished. And the activity of the gene product thereof is the activity of GM02LC38418-protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “GM02LC38418-protein”, especially from Glycine max or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said GM02LC38418, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said GM02LC38418, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said GM02LC38418, or a functional equivalent or a homolog thereof as depicted in column 8 of Table II, application no. 8, preferably a homolog or functional equivalent as depicted in column 8 of Table II B, application no. 8, and being depicted in the same respective line as said GM02LC38418, and preferably the activity is increased non-targeted.

Accordingly, in one embodiment, the molecule which activity is to be increased in the process of the invention is the gene product with an activity as a “GM02LC38418-protein”, preferably it is the molecule of section (a) or (b) of this paragraph.

In particular, it was observed that in plants, especially in Arabidopsis thaliana, increasing or generating the activity of a gene product non-targeted with the activity of a “GM02LC38418-protein”, preferably being encoded by a gene comprising the nucleic acid sequence SEQ ID NO. 116312, preferably the coding region thereof, conferred the production of or the increase in linoleic acid compared with the wild type control.

The nucleic acid sequence of Sll1032 from Synechocystis sp., e.g. as shown in the respective line in column 5 of Table I, application no. 8, has been published in C. And the activity of the gene product thereof is the activity of sll1032-protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “sll1032-protein”, especially from Synechocystis sp. or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said Sll1032, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said Sll1032, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said Sll1032, or a functional equivalent or a homolog thereof as depicted in column 8 of Table II, application no. 8, preferably a homolog or functional equivalent as depicted in column 8 of Table II B, application no. 8, and being depicted in the same respective line as said Sll1032, and preferably the activity is increased mitochondrial.

Accordingly, in one embodiment, the molecule which activity is to be increased in the process of the invention is the gene product with an activity as a “sll1032-protein”, preferably it is the molecule of section (a) or (b) of this paragraph.

In particular, it was observed that in plants, especially in Arabidopsis thaliana, increasing or generating the activity of a gene product mitochondrial with the activity of a “sll1032-protein”, preferably being encoded by a gene comprising the nucleic acid sequence SEQ ID NO. 85723, preferably the coding region thereof, conferred the production of or the increase in linoleic acid compared with the wild type control.

The nucleic acid sequence of Sll1056 from Synechocystis sp., e.g. as shown in the respective line in column 5 of Table I, application no. 8, has been published in C. And the activity of the gene product thereof is the activity of phosphoribosylformyl glycinamidine synthase subunit.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “phosphoribosylformyl glycinamidine synthase subunit”, especially from Synechocystis sp. or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said Sll1056, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said Sll1056, and preferably the activity is increased plastidic, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said Sll1056, or a functional equivalent or a homolog thereof as depicted in column 8 of Table II, application no. 8, preferably a homolog or functional equivalent as depicted in column 8 of Table II B, application no. 8, and being depicted in the same respective line as said Sll1056, and preferably the activity is increased plastidic.

Accordingly, in one embodiment, the molecule which activity is to be increased in the process of the invention is the gene product with an activity as a “phosphoribosylformyl glycinamidine synthase subunit”, preferably it is the molecule of section (a) or (b) of this paragraph.

In particular, it was observed that in plants, especially in Arabidopsis thaliana, increasing or generating the activity of a gene product plastidic with the activity of a “phosphoribosylformyl glycinamidine synthase subunit”, preferably being encoded by a gene comprising the nucleic acid sequence SEQ ID NO. 52660, preferably the coding region thereof, conferred the production of or the increase in linoleic acid compared with the wild type control.

The nucleic acid sequence of Sll1443 from Synechocystis sp., e.g. as shown in the respective line in column 5 of Table I, application no. 8, has been published in C. And the activity of the gene product thereof is the activity of CTP synthetase.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “CTP synthetase”, especially from Synechocystis sp. or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said Sll1443, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said Sll1443, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said Sll1443, or a functional equivalent or a homolog thereof as depicted in column 8 of Table II, application no. 8, preferably a homolog or functional equivalent as depicted in column 8 of Table II B, application no. 8, and being depicted in the same respective line as said Sll1443, and preferably the activity is increased mitochondrial.

Accordingly, in one embodiment, the molecule which activity is to be increased in the process of the invention is the gene product with an activity as a “CTP synthetase”, preferably it is the molecule of section (a) or (b) of this paragraph.

In particular, it was observed that in plants, especially in Arabidopsis thaliana, increasing or generating the activity of a gene product mitochondrial with the activity of a “CTP synthetase”, preferably being encoded by a gene comprising the nucleic acid sequence SEQ ID NO. 53878, preferably the coding region thereof, conferred the production of or the increase in linoleic acid compared with the wild type control.

The nucleic acid sequence of Sll1498 from Synechocystis sp., e.g. as shown in the respective line in column 5 of Table I, application no. 8, has been published in C. And the activity of the gene product thereof is the activity of carbamoyl-phosphate synthase subunit.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “carbamoyl-phosphate synthase subunit”, especially from Synechocystis sp. or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said Sll1498, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said Sll1498, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said Sll1498, or a functional equivalent or a homolog thereof as depicted in column 8 of Table II, application no. 8, preferably a homolog or functional equivalent as depicted in column 8 of Table II B, application no. 8, and being depicted in the same respective line as said Sll1498, and preferably the activity is increased non-targeted.

Accordingly, in one embodiment, the molecule which activity is to be increased in the process of the invention is the gene product with an activity as a “carbamoyl-phosphate synthase subunit”, preferably it is the molecule of section (a) or (b) of this paragraph.

In particular, it was observed that in plants, especially in Arabidopsis thaliana, increasing or generating the activity of a gene product non-targeted with the activity of a “carbamoylphosphate synthase subunit”, preferably being encoded by a gene comprising the nucleic acid sequence SEQ ID NO. 95047, preferably the coding region thereof, conferred the production of or the increase in linoleic acid compared with the wild type control.

The nucleic acid sequence of Sll1917 from Synechocystis sp., e.g. as shown in the respective line in column 5 of Table I, application no. 8, has been published in C. And the activity of the gene product thereof is the activity of coproporphyrinogen III oxidase.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “coproporphyrinogen III oxidase”, especially from Synechocystis sp. or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said Sll1917, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said Sll1917, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said Sll1917, or a functional equivalent or a homolog thereof as depicted in column 8 of Table II, application no. 8, preferably a homolog or functional equivalent as depicted in column 8 of Table II B, application no. 8, and being depicted in the same respective line as said Sll1917, and preferably the activity is increased mitochondrial.

Accordingly, in one embodiment, the molecule which activity is to be increased in the process of the invention is the gene product with an activity as a “coproporphyrinogen III oxidase”, preferably it is the molecule of section (a) or (b) of this paragraph.

In particular, it was observed that in plants, especially in Arabidopsis thaliana, increasing or generating the activity of a gene product mitochondrial with the activity of a “coproporphyrinogen III oxidase”, preferably being encoded by a gene comprising the nucleic acid sequence SEQ ID NO. 11471, preferably the coding region thereof, conferred the production of or the increase in linoleic acid compared with the wild type control.

The nucleic acid sequence of Sll1920 from Synechocystis sp., e.g. as shown in the respective line in column 5 of Table I, application no. 8, has been published in C. And the activity of the gene product thereof is the activity of cation-transporting ATPase.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “cation-transporting ATPase”, especially from Synechocystis sp. or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said Sll1920, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said Sll1920, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said Sll1920, or a functional equivalent or a homolog thereof as depicted in column 8 of Table II, application no. 8, preferably a homolog or functional equivalent as depicted in column 8 of Table II B, application no. 8, and being depicted in the same respective line as said Sll1920, and preferably the activity is increased mitochondrial.

Accordingly, in one embodiment, the molecule which activity is to be increased in the process of the invention is the gene product with an activity as a “cation-transporting ATPase”, preferably it is the molecule of section (a) or (b) of this paragraph.

In particular, it was observed that in plants, especially in Arabidopsis thaliana, increasing or generating the activity of a gene product mitochondrial with the activity of a “cation-transporting ATPase”, preferably being encoded by a gene comprising the nucleic acid sequence SEQ ID NO. 55385, preferably the coding region thereof, conferred the production of or the increase in linoleic acid compared with the wild type control.

The nucleic acid sequence of Slr0338 from Synechocystis sp., e.g. as shown in the respective line in column 5 of Table I, application no. 8, has been published in C. And the activity of the gene product thereof is the activity of oxidoreductase.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “oxidoreductase”, especially from Synechocystis sp. or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said Slr0338, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said Slr0338, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said Slr0338, or a functional equivalent or a homolog thereof as depicted in column 8 of Table II, application no. 8, preferably a homolog or functional equivalent as depicted in column 8 of Table II B, application no. 8, and being depicted in the same respective line as said Slr0338, and preferably the activity is increased non-targeted.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 11990, preferably the coding region thereof, conferred the production of or the increase in linoleic acid compared with the wild type control.

The nucleic acid sequence of Slr0966 from Synechocystis sp., e.g. as shown in the respective line in column 5 of Table I, application no. 8, has been published in C. And the activity of the gene product thereof is the activity of tryptophan synthase alpha chain.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “tryptophan synthase alpha chain”, especially from Synechocystis sp. or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said Slr0966, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said Slr0966, and preferably the activity is increased plastidic, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said Slr0966, or a functional equivalent or a homolog thereof as depicted in column 8 of Table II, application no. 8, preferably a homolog or functional equivalent as depicted in column 8 of Table II B, application no. 8, and being depicted in the same respective line as said Slr0966, and preferably the activity is increased plastidic.

Accordingly, in one embodiment, the molecule which activity is to be increased in the process of the invention is the gene product with an activity as a “tryptophan synthase alpha chain”, preferably it is the molecule of section (a) or (b) of this paragraph.

In particular, it was observed that in plants, especially in Arabidopsis thaliana, increasing or generating the activity of a gene product plastidic with the activity of a “tryptophan synthase alpha chain”, preferably being encoded by a gene comprising the nucleic acid sequence SEQ ID NO. 87655, preferably the coding region thereof, conferred the production of or the increase in linoleic acid compared with the wild type control.

The nucleic acid sequence of Slr1099 from Synechocystis sp., e.g. as shown in the respective line in column 5 of Table I, application no. 8, has been published in C. And the activity of the gene product thereof is the activity of aromatic acid decarboxylase.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “aromatic acid decarboxylase”, especially from Synechocystis sp. or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said Slr1099, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said Slr1099, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said Slr1099, or a functional equivalent or a homolog thereof as depicted in column 8 of Table II, application no. 8, preferably a homolog or functional equivalent as depicted in column 8 of Table II B, application no. 8, and being depicted in the same respective line as said Slr1099, and preferably the activity is increased mitochondrial.

Accordingly, in one embodiment, the molecule which activity is to be increased in the process of the invention is the gene product with an activity as a “aromatic acid decarboxylase”, preferably it is the molecule of section (a) or (b) of this paragraph.

In particular, it was observed that in plants, especially in Arabidopsis thaliana, increasing or generating the activity of a gene product mitochondrial with the activity of a “aromatic acid decarboxylase”, preferably being encoded by a gene comprising the nucleic acid sequence SEQ ID NO. 116341, preferably the coding region thereof, conferred the production of or the increase in linoleic acid compared with the wild type control.

The nucleic acid sequence of Slr1250 from Synechocystis sp., e.g. as shown in the respective line in column 5 of Table I, application no. 8, has been published in C. And the activity of the gene product thereof is the activity of phosphate import ATP-binding protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “phosphate import ATP-binding protein”, especially from Synechocystis sp. or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said Slr1250, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said Slr1250, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said Slr1250, or a functional equivalent or a homolog thereof as depicted in column 8 of Table II, application no. 8, preferably a homolog or functional equivalent as depicted in column 8 of Table II B, application no. 8, and being depicted in the same respective line as said Slr1250, and preferably the activity is increased mitochondrial.

Accordingly, in one embodiment, the molecule which activity is to be increased in the process of the invention is the gene product with an activity as a “phosphate import ATP-binding protein”, preferably it is the molecule of section (a) or (b) of this paragraph.

In particular, it was observed that in plants, especially in Arabidopsis thaliana, increasing or generating the activity of a gene product mitochondrial with the activity of a “phosphate import ATP-binding protein”, preferably being encoded by a gene comprising the nucleic acid sequence SEQ ID NO. 116460, preferably the coding region thereof, conferred the production of or the increase in linoleic acid compared with the wild type control.

The nucleic acid sequence of Slr1269 from Synechocystis sp., e.g. as shown in the respective line in column 5 of Table I, application no. 8, has been published in C. And the activity of the gene product thereof is the activity of gamma-glutamyltranspeptidase.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “gamma-glutamyltranspeptidase”, especially from Synechocystis sp. or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said Slr1269, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said Slr1269, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said Slr1269, or a functional equivalent or a homolog thereof as depicted in column 8 of Table II, application no. 8, preferably a homolog or functional equivalent as depicted in column 8 of Table II B, application no. 8, and being depicted in the same respective line as said Slr1269, and preferably the activity is increased non-targeted.

Accordingly, in one embodiment, the molecule which activity is to be increased in the process of the invention is the gene product with an activity as a “gamma-glutamyltranspeptidase”, preferably it is the molecule of section (a) or (b) of this paragraph.

In particular, it was observed that in plants, especially in Arabidopsis thaliana, increasing or generating the activity of a gene product non-targeted with the activity of a “gammaglutamyltranspeptidase”, preferably being encoded by a gene comprising the nucleic acid sequence SEQ ID NO. 58058, preferably the coding region thereof, conferred the production of or the increase in linoleic acid compared with the wild type control.

The nucleic acid sequence of Slr1312 from Synechocystis sp., e.g. as shown in the respective line in column 5 of Table I, application no. 8, has been published in C. And the activity of the gene product thereof is the activity of arginine decarboxylase.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “arginine decarboxylase”, especially from Synechocystis sp. or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said Slr1312, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said Slr1312, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said Slr1312, or a functional equivalent or a homolog thereof as depicted in column 8 of Table II, application no. 8, preferably a homolog or functional equivalent as depicted in column 8 of Table II B, application no. 8, and being depicted in the same respective line as said Slr1312, and preferably the activity is increased non-targeted.

Accordingly, in one embodiment, the molecule which activity is to be increased in the process of the invention is the gene product with an activity as a “arginine decarboxylase”, preferably it is the molecule of section (a) or (b) of this paragraph.

In particular, it was observed that in plants, especially in Arabidopsis thaliana, increasing or generating the activity of a gene product non-targeted with the activity of a “arginine decarboxylase”, preferably being encoded by a gene comprising the nucleic acid sequence SEQ ID NO. 58324, preferably the coding region thereof, conferred the production of or the increase in linoleic acid compared with the wild type control.

The nucleic acid sequence of Slr1350 from Synechocystis sp., e.g. as shown in the respective line in column 5 of Table I, application no. 8, has been published in C. And the activity of the gene product thereof is the activity of fatty acid desaturase.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “fatty acid desaturase”, especially from Synechocystis sp. or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said Slr1350, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said Slr1350, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said Slr1350, or a functional equivalent or a homolog thereof as depicted in column 8 of Table II, application no. 8, preferably a homolog or functional equivalent as depicted in column 8 of Table II B, application no. 8, and being depicted in the same respective line as said Slr1350, and preferably the activity is increased non-targeted.

Accordingly, in one embodiment, the molecule which activity is to be increased in the process of the invention is the gene product with an activity as a “fatty acid desaturase”, preferably it is the molecule of section (a) or (b) of this paragraph.

In particular, it was observed that in plants, especially in Arabidopsis thaliana, increasing or generating the activity of a gene product non-targeted with the activity of a “fatty acid desaturase”, preferably being encoded by a gene comprising the nucleic acid sequence SEQ ID NO. 116713, preferably the coding region thereof, conferred the production of or the increase in linoleic acid compared with the wild type control.

The nucleic acid sequence of Slr2072 from Synechocystis sp., e.g. as shown in the respective line in column 5 of Table I, application no. 8, has been published in C. And the activity of the gene product thereof is the activity of threonine dehydratase.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “threonine dehydratase”, especially from Synechocystis sp. or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said Slr2072, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said Slr2072, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said Slr2072, or a functional equivalent or a homolog thereof as depicted in column 8 of Table II, application no. 8, preferably a homolog or functional equivalent as depicted in column 8 of Table II B, application no. 8, and being depicted in the same respective line as said Slr2072, and preferably the activity is increased mitochondrial.

Accordingly, in one embodiment, the molecule which activity is to be increased in the process of the invention is the gene product with an activity as a “threonine dehydratase”, preferably it is the molecule of section (a) or (b) of this paragraph.

In particular, it was observed that in plants, especially in Arabidopsis thaliana, increasing or generating the activity of a gene product mitochondrial with the activity of a “threonine dehydratase”, preferably being encoded by a gene comprising the nucleic acid sequence SEQ ID NO. 12341, preferably the coding region thereof, conferred the production of or the increase in linoleic acid compared with the wild type control.

The nucleic acid sequence of TTC0490 from Thermus thermophilus, e.g. as shown in the respective line in column 5 of Table I, application no. 8, has been published in E. And the activity of the gene product thereof is the activity of TTC0490-protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “TTC0490-protein”, especially from Thermus thermophilus or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said TTC0490, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said TTC0490, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said TTC0490, or a functional equivalent or a homolog thereof as depicted in column 8 of Table II, application no. 8, preferably a homolog or functional equivalent as depicted in column 8 of Table II B, application no. 8, and being depicted in the same respective line as said TTC0490, and preferably the activity is increased non-targeted.

Accordingly, in one embodiment, the molecule which activity is to be increased in the process of the invention is the gene product with an activity as a “TTC0490-protein”, preferably it is the molecule of section (a) or (b) of this paragraph.

In particular, it was observed that in plants, especially in Arabidopsis thaliana, increasing or generating the activity of a gene product non-targeted with the activity of a “TTC0490-protein”, preferably being encoded by a gene comprising the nucleic acid sequence SEQ ID NO. 116848, preferably the coding region thereof, conferred the production of or the increase in linoleic acid compared with the wild type control.

The nucleic acid sequence of TTC0917 from Thermus thermophilus, e.g. as shown in the respective line in column 5 of Table I, application no. 8, has been published in E. And the activity of the gene product thereof is the activity of metal-dependent hydrolase.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “metal-dependent hydrolase”, especially from Thermus thermophilus or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said TTC0917, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said TTC0917, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said TTC0917, or a functional equivalent or a homolog thereof as depicted in column 8 of Table II, application no. 8, preferably a homolog or functional equivalent as depicted in column 8 of Table II B, application no. 8, and being depicted in the same respective line as said TTC0917, and preferably the activity is increased non-targeted.

Accordingly, in one embodiment, the molecule which activity is to be increased in the process of the invention is the gene product with an activity as a “metal-dependent hydrolase”, preferably it is the molecule of section (a) or (b) of this paragraph.

In particular, it was observed that in plants, especially in Arabidopsis thaliana, increasing or generating the activity of a gene product non-targeted with the activity of a “metal-dependent hydrolase”, preferably being encoded by a gene comprising the nucleic acid sequence SEQ ID NO. 61553, preferably the coding region thereof, conferred the production of or the increase in linoleic acid compared with the wild type control.

The nucleic acid sequence of TTC1193 from Thermus thermophilus, e.g. as shown in the respective line in column 5 of Table I, application no. 8, has been published in E. And the activity of the gene product thereof is the activity of multiple antibiotic resistance protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “multiple antibiotic resistance protein”, especially from Thermus thermophilus or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said TTC1193, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said TTC1193, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said TTC1193, or a functional equivalent or a homolog thereof as depicted in column 8 of Table II, application no. 8, preferably a homolog or functional equivalent as depicted in column 8 of Table II B, application no. 8, and being depicted in the same respective line as said TTC1193, and preferably the activity is increased non-targeted.

Accordingly, in one embodiment, the molecule which activity is to be increased in the process of the invention is the gene product with an activity as a “multiple antibiotic resistance protein”, preferably it is the molecule of section (a) or (b) of this paragraph.

In particular, it was observed that in plants, especially in Arabidopsis thaliana, increasing or generating the activity of a gene product non-targeted with the activity of a “multiple antibiotic resistance protein”, preferably being encoded by a gene comprising the nucleic acid sequence SEQ ID NO. 61723, preferably the coding region thereof, conferred the production of or the increase in linoleic acid compared with the wild type control.

The nucleic acid sequence of Yal044c from Saccharomyces cerevisiae, e.g. as shown in the respective line in column 5 of Table I, application no. 8, has been published in A. And the activity of the gene product thereof is the activity of glycin cleavage system protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “glycin cleavage system protein”, especially from Saccharomyces cerevisiae or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said Yal044c, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said Yal044c, and preferably the activity is increased plastidic, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said Yal044c, or a functional equivalent or a homolog thereof as depicted in column 8 of Table II, application no. 8, preferably a homolog or functional equivalent as depicted in column 8 of Table II B, application no. 8, and being depicted in the same respective line as said Yal044c, and preferably the activity is increased plastidic.

Accordingly, in one embodiment, the molecule which activity is to be increased in the process of the invention is the gene product with an activity as a “glycin cleavage system protein”, preferably it is the molecule of section (a) or (b) of this paragraph.

In particular, it was observed that in plants, especially in Arabidopsis thaliana, increasing or generating the activity of a gene product plastidic with the activity of a “glycin cleavage system protein”, preferably being encoded by a gene comprising the nucleic acid sequence SEQ ID NO. 116948, preferably the coding region thereof, conferred the production of or the increase in linoleic acid compared with the wild type control.

The nucleic acid sequence of Ydl159w from Saccharomyces cerevisiae, e.g. as shown in the respective line in column 5 of Table I, application no. 8, has been published in A. And the activity of the gene product thereof is the activity of protein kinase.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “protein kinase”, especially from Saccharomyces cerevisiae or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said Ydl159w, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said Ydl159w, and preferably the activity is increased plastidic, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said Ydl159w, or a functional equivalent or a homolog thereof as depicted in column 8 of Table II, application no. 8, preferably a homolog or functional equivalent as depicted in column 8 of Table II B, application no. 8, and being depicted in the same respective line as said Ydl159w, and preferably the activity is increased plastidic.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product plastidic with the activity of a “protein kinase”, preferably being encoded by a gene comprising the nucleic acid sequence SEQ ID NO. 14275, preferably the coding region thereof, conferred the production of or the increase in linoleic acid compared with the wild type control.

The nucleic acid sequence of Ydr158w from Saccharomyces cerevisiae, e.g. as shown in the respective line in column 5 of Table I, application no. 8, has been published in A. And the activity of the gene product thereof is the activity of aspartate-semialdehyde dehydrogenase.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “aspartate-semialdehyde dehydrogenase”, especially from Saccharomyces cerevisiae or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said Ydr158w, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said Ydr158w, and preferably the activity is increased plastidic, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said Ydr158w, or a functional equivalent or a homolog thereof as depicted in column 8 of Table II, application no. 8, preferably a homolog or functional equivalent as depicted in column 8 of Table II B, application no. 8, and being depicted in the same respective line as said Ydr158w, and preferably the activity is increased plastidic.

Accordingly, in one embodiment, the molecule which activity is to be increased in the process of the invention is the gene product with an activity as a “aspartate-semialdehyde dehydrogenase”, preferably it is the molecule of section (a) or (b) of this paragraph.

In particular, it was observed that in plants, especially in Arabidopsis thaliana, increasing or generating the activity of a gene product plastidic with the activity of a “aspartate-semialdehyde dehydrogenase”, preferably being encoded by a gene comprising the nucleic acid sequence SEQ ID NO. 117352, preferably the coding region thereof, conferred the production of or the increase in linoleic acid compared with the wild type control.

The nucleic acid sequence of Ydr507c from Saccharomyces cerevisiae, e.g. as shown in the respective line in column 5 of Table I, application no. 8, has been published in A. And the activity of the gene product thereof is the activity of ydr507c-protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “ydr507c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said Ydr507c, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said Ydr507c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said Ydr507c, or a functional equivalent or a homolog thereof as depicted in column 8 of Table II, application no. 8, preferably a homolog or functional equivalent as depicted in column 8 of Table II B, application no. 8, and being depicted in the same respective line as said Ydr507c, and preferably the activity is increased non-targeted.

Accordingly, in one embodiment, the molecule which activity is to be increased in the process of the invention is the gene product with an activity as a “ydr507c-protein”, preferably it is the molecule of section (a) or (b) of this paragraph.

In particular, it was observed that in plants, especially in Arabidopsis thaliana, increasing or generating the activity of a gene product non-targeted with the activity of a “ydr507c-protein”, preferably being encoded by a gene comprising the nucleic acid sequence SEQ ID NO. 90165, preferably the coding region thereof, conferred the production of or the increase in linoleic acid compared with the wild type control.

The nucleic acid sequence of Ygr104c from Saccharomyces cerevisiae, e.g. as shown in the respective line in column 5 of Table I, application no. 8, has been published in A. And the activity of the gene product thereof is the activity of YGR104C-protein.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “YGR104C-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said Ygr104c, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said Ygr104c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said Ygr104c, or a functional equivalent or a homolog thereof as depicted in column 8 of Table II, application no. 8, preferably a homolog or functional equivalent as depicted in column 8 of Table II B, application no. 8, and being depicted in the same respective line as said Ygr104c, and preferably the activity is increased non-targeted.

Accordingly, in one embodiment, the molecule which activity is to be increased in the process of the invention is the gene product with an activity as a “YGR104C-protein”, preferably it is the molecule of section (a) or (b) of this paragraph.

In particular, it was observed that in plants, especially in Arabidopsis thaliana, increasing or generating the activity of a gene product non-targeted with the activity of a “YGR104C-protein”, preferably being encoded by a gene comprising the nucleic acid sequence SEQ ID NO. 117482, preferably the coding region thereof, conferred the production of or the increase in linoleic acid compared with the wild type control.

The nucleic acid sequence of Yjl073w from Saccharomyces cerevisiae, e.g. as shown in the respective line in column 5 of Table I, application no. 8, has been published in A. And the activity of the gene product thereof is the activity of DnaJ-like chaperone.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “DnaJ-like chaperone”, especially from Saccharomyces cerevisiae or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said Yjl073w, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said Yjl073w, and preferably the activity is increased plastidic, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said Yj1073w, or a functional equivalent or a homolog thereof as depicted in column 8 of Table II, application no. 8, preferably a homolog or functional equivalent as depicted in column 8 of Table II B, application no. 8, and being depicted in the same respective line as said Yjl073w, and preferably the activity is increased plastidic.

Accordingly, in one embodiment, the molecule which activity is to be increased in the process of the invention is the gene product with an activity as a “DnaJ-like chaperone”, preferably it is the molecule of section (a) or (b) of this paragraph.

In particular, it was observed that in plants, especially in Arabidopsis thaliana, increasing or generating the activity of a gene product plastidic with the activity of a “DnaJ-like chaperone”, preferably being encoded by a gene comprising the nucleic acid sequence SEQ ID NO. 64964, preferably the coding region thereof, conferred the production of or the increase in linoleic acid compared with the wild type control.

The nucleic acid sequence of Ylr304c from Saccharomyces cerevisiae, e.g. as shown in the respective line in column 5 of Table I, application no. 8, has been published in A. And the activity of the gene product thereof is the activity of aconitate hydratase.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “aconitate hydratase”, especially from Saccharomyces cerevisiae or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said Ylr304c, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said Ylr304c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said Ylr304c, or a functional equivalent or a homolog thereof as depicted in column 8 of Table II, application no. 8, preferably a homolog or functional equivalent as depicted in column 8 of Table II B, application no. 8, and being depicted in the same respective line as said Ylr304c, and preferably the activity is increased non-targeted.

Accordingly, in one embodiment, the molecule which activity is to be increased in the process of the invention is the gene product with an activity as a “aconitate hydratase”, preferably it is the molecule of section (a) or (b) of this paragraph.

In particular, it was observed that in plants, especially in Arabidopsis thaliana, increasing or generating the activity of a gene product non-targeted with the activity of a “aconitate hydratase”, preferably being encoded by a gene comprising the nucleic acid sequence SEQ ID NO. 66772, preferably the coding region thereof, conferred the production of or the increase in linoleic acid compared with the wild type control.

The nucleic acid sequence of Ynl064c from Saccharomyces cerevisiae, e.g. as shown in the respective line in column 5 of Table I, application no. 8, has been published in A. And the activity of the gene product thereof is the activity of molecular chaperone.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “molecular chaperone”, especially from Saccharomyces cerevisiae or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said Ynl064c, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said YnI064c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said Ynl064c, or a functional equivalent or a homolog thereof as depicted in column 8 of Table II, application no. 8, preferably a homolog or functional equivalent as depicted in column 8 of Table II B, application no. 8, and being depicted in the same respective line as said Ynl064c, and preferably the activity is increased non-targeted.

Accordingly, in one embodiment, the molecule which activity is to be increased in the process of the invention is the gene product with an activity as a “molecular chaperone”, preferably it is the molecule of section (a) or (b) of this paragraph.

In particular, it was observed that in plants, especially in Arabidopsis thaliana, increasing or generating the activity of a gene product non-targeted with the activity of a “molecular chaperone”, preferably being encoded by a gene comprising the nucleic acid sequence SEQ ID NO. 117495, preferably the coding region thereof, conferred the production of or the increase in linoleic acid compared with the wild type control.

The nucleic acid sequence of Ypl271w from Saccharomyces cerevisiae, e.g. as shown in the respective line in column 5 of Table I, application no. 8, has been published in A. And the activity of the gene product thereof is the activity of ATPase epsilon subunit.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “ATPase epsilon subunit”, especially from Saccharomyces cerevisiae or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said Ypl271w, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said Ypl271w, and preferably the activity is increased plastidic, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said Ypl271w, or a functional equivalent or a homolog thereof as depicted in column 8 of Table II, application no. 8, preferably a homolog or functional equivalent as depicted in column 8 of Table II B, application no. 8, and being depicted in the same respective line as said Ypl271w, and preferably the activity is increased plastidic.

Accordingly, in one embodiment, the molecule which activity is to be increased in the process of the invention is the gene product with an activity as a “ATPase epsilon subunit”, preferably it is the molecule of section (a) or (b) of this paragraph.

In particular, it was observed that in plants, especially in Arabidopsis thaliana, increasing or generating the activity of a gene product plastidic with the activity of a “ATPase epsilon subunit”, preferably being encoded by a gene comprising the nucleic acid sequence SEQ ID NO. 118212, preferably the coding region thereof, conferred the production of or the increase in linoleic acid compared with the wild type control.

The nucleic acid sequence of 59582753_SOYBEAN from Glycine max, e.g. as shown in the respective line in column 5 of Table I, application no. 8, is unpublished. And the activity of the gene product thereof is the activity of glucose-6-phosphate 1-dehydrogenase.

Accordingly, in one embodiment, the process of the present invention for producing linoleic acid in a non-human organism, like a microorganism or a plant or a part thereof, comprises increasing or generating the activity of a gene product with the activity of a gene product conferring the activity of “glucose-6-phosphate 1-dehydrogenase”, especially from Glycine max or its functional equivalent or its homolog, e.g. the increase of
(a) a gene product of a gene comprising the nucleic acid molecule as shown in the respective line in column 5 of Table I, application no. 8, preferably the coding region thereof, or a homolog or a fragment thereof, and being depicted in the same respective line as said 59582753_SOYBEAN, or a functional equivalent or a homolog thereof as shown in column 8 of Table I, application no. 8, preferably the coding region thereof, particularly a homolog or functional equivalent as shown in column 8 of Table I B, application no. 8, and being depicted in the same respective line as said 59582753_SOYBEAN, and preferably the activity is increased plastidic, or
(b) a polypeptide comprising a polypeptide, a consensus sequence or at least a polypeptide motif as shown in the respective line in column 5 of Table II or in column 8 of Table IV, application no. 8, respectively, and being depicted in the same respective line as said 59582753_SOYBEAN, or a functional equivalent or a homolog thereof as depicted in column 8 of Table II, application no. 8, preferably a homolog or functional equivalent as depicted in column 8 of Table II B, application no. 8, and being depicted in the same respective line as said 59582753_SOYBEAN, and preferably the activity is increased plastidic.

Accordingly, in one embodiment, the molecule which activity is to be increased in the process of the invention is the gene product with an activity as a “glucose-6-phosphate 1-dehydrogenase”, preferably it is the molecule of section (a) or (b) of this paragraph.

In particular, it was observed that in plants, especially in Arabidopsis thaliana, increasing or generating the activity of a gene product plastidic with the activity of a “glucose-6-phosphate 1-dehydrogenase”, preferably being encoded by a gene comprising the nucleic acid sequence SEQ ID NO. 16883, preferably the coding region thereof, conferred the production of or the increase in linoleic acid compared with the wild type control.

It was further observed that increasing or generating the activity of a FCRP gene shown in Table d, e.g. a nucleic acid molecule derived from the coding region of the nucleic acid molecule shown in Table d in A. thaliana conferred the production of or the increase in fine chemical, compared with the wild type control. Thus, in one embodiment, a nucleic acid molecule, preferably the coding region thereof, indicated in Table d or its homolog as indicated in the respective line in Table I or the expression product is used in the method of the present invention to generate or to increase the production of fine chemical in a non-human organism, like a microorganism or a plant, compared with the wild type control.

In this context, the fine chemical amount in a cell, in a non-human organism, like a plant or a microorganism or a part thereof, is increased by 1%, 3%, 5%, 10%, 20%, 30%, 40%, 50%, 60%, 70%, 80%, 90%, 100%, 120, 150% or 200% or more.

The fine chemical can be contained in the organism either in its free form and/or bound, e.g. in triglycerides, phospholipids, lipids, oils and/or fatsor mixtures thereof. Accordingly, in one embodiment, the amount of the free form in a cell, in a non-human organism, like a plant or a microorganism or part thereof, is increased by 1%, 3%, 5%, 10%, 20%, 30%, 40%, 50%, 60%, 70%, 80%, 90%, 100%, 120, 150% or 200% or more. Accordingly, in another embodiment, the amount of the bound fine chemical in a cell, in a non-human organism, like a plant or a microorganism or part thereof, is increased byl %, 3%, 5%, 10%, 20%, 30%, 40%, 50%, 60%, 70%, 80%, 90%, 100%, 120, 150% or 200% or more.

A protein having an activity conferring an increase in the amount or level of the fine chemical linoleic acid, upon targeting to the plastids or mitochondria or upon non-targeting, preferably has the structure of the respective polypeptide described herein, in particular of the polypeptides comprising the consensus sequence or at least one polypeptide motifs as shown in the respective line in Table IV, application no. 8, column 8 or of the polypeptide comprising an amino acid sequence as disclosed in the respective line in Table II, application no. 8, columns 5 or 8, or homologs or fragments thereof as described herein, or is encoded by the nucleic acid molecule characterized herein or the nucleic acid molecule according to the invention, for example by the nucleic acid molecule as shown in the respective line in Table I, application no. 8, columns 5 or 8, preferably the coding region thereof, or homologs or fragments thereof and has the herein mentioned activity.

Owing to the biological activity of the proteins which are used in the process according to the invention and which are encoded by nucleic acid molecules according to the invention, it is possible to produce compositions comprising the fine chemical, i.e. an increased amount of the fine chemical free or bound, e.g. in triglyceride, phospholipid,lipid, oil and/or fat compositions. Depending on the choice of the non-human organism used for the process according to the present invention, for example a microorganism or a plant, compositions or mixtures comprising the fine chemical can be produced.

The term “expression” refers to the transcription and/or translation of a codogenic gene segment or gene. As a rule, the resulting product is an mRNA or a protein. However, expression products can also include functional RNAs such as, for example, antisense, nucleic acids, tRNAs, snRNAs, rRNAs, RNAi, siRNA, ribozymes etc. Expression may be systemic, local or temporal, for example limited to certain cell types, tissues organs or organelles or time periods.

In one embodiment, the process of the present invention comprises one or more of the following steps
(a) stabilizing a protein conferring the generation or increased expression of a protein encoded by the nucleic acid molecule of the invention or of the polypeptide of the invention, e.g. of a polypeptide having the activity selected from the group consisting of 3-ketoacyl-CoA thiolase, 3-oxoacyl-[acyl-carrier-protein] synthase, AAS bifunctional protein, ABC transporter permease protein, acid shock protein, aconitate hydratase, arginine decarboxylase, aromatic acid decarboxylase, aspartate-semialdehyde dehydrogenase, At1g19800-protein, At2g45420-protein, ATPase epsilon subunit, b0801-protein, b1330-protein, b1470-protein, b1670-protein, b1837-protein, b2032-protein, b2099-protein, b2107-protein, b2399-protein, b2474-protein, b2613-protein, b2682-protein, b2849-protein, b2909-protein, beta-hydroxylase, carbamoyl-phosphate synthase subunit, cation-transporting ATPase, CCAAT-binding transcription factor, chlorophyllase, colanic acid biosynthesis protein, coproporphyrinogen III oxidase, CTP synthetase, DnaJ-like chaperone, electron transport complex protein, ethanolamine utilization protein, fatty acid desaturase, Fe—S subunit of oxidoreductase, flavodoxin, fumarate hydratase, gamma-glutamyltranspeptidase, glucose-6-phosphate 1-dehydrogenase, glutaredoxin, glycin cleavage system protein, GM02LC38418-protein, heat shock transcription factor, hydrolase, lipoprotein precursor, membrane transport protein, metal-dependent hydrolase, molecular chaperone, monothiol glutaredoxin, multiple antibiotic resistance protein, outer membrane receptor protein precursor, oxidoreductase, phosphate import ATP-binding protein, phosphoribosylformyl glycinamidine synthase subunit, phosphotriesterase, protein kinase, protein phosphatase, pyruvate kinase, recombinase A, sll1032-protein, sn-glycerol-3-phosphate transport system permease protein, ThiF family protein, threonine dehydratase, threonine efflux protein, transcription factor, transcriptional regulator protein, tryptophan synthase alpha chain, TTC0490-protein, xanthine dehydrogenase, ydr507c-protein, and YGR104C-protein, or of a polypeptide as indicated in the respective line in Table II, application no. 8, columns 5 or 8, or its homologs or fragments, and conferring the production of or an increase in linoleic acid, respectively; and/or
(b) stabilizing a mRNA conferring the generation or increased expression of a FCRP, e.g. protein encoded by the nucleic acid molecule of the invention or its homologs or fragments, or of a mRNA encoding the polypeptide of the present invention having the hereinmentioned activity selected from the group consisting of said activities mentioned in (a) and conferring the production of or an increase in linoleic acid, respectively; and/or
(c) increasing the specific activity of a protein conferring the increased expression of a FCRP, e.g. a protein encoded by the nucleic acid molecule of the invention or of the polypeptide of the present invention having herein-mentioned linoleic acid generating or increasing activity, respectively, e.g. of a polypeptide having the activity of a protein as indicated in the respective line in Table II, application no. 8, columns 5 or 8, or its homologs or fragments, or decreasing the inhibitory regulation of the polypeptide of the invention; and/or
(d) generating or increasing the expression of an endogenous or artificial transcription factor mediating the expression of a protein conferring the generation or increased expression of a FCRP, e.g. a protein encoded by the nucleic acid molecule of the invention or of the polypeptide of the invention having herein-mentioned activity selected from the group consisting of said activities mentioned in (a) and conferring a linoleic acid increasing activity, respectively, e.g. of a polypeptide having the activity of a protein as indicated in the respective line in Table II, application no. 8, columns 5 or 8 or its homologs; and/or
(e) stimulating activity of a protein conferring the increased expression of a FCRP, e.g. a protein encoded by the nucleic acid molecule of the present invention or a polypeptide of the present invention having herein-mentioned activity selected from the group consisting of said activities mentioned in (a) and conferring a linoleic acid increasing activity, respectively, e.g. of a polypeptide having the activity of a protein as indicated in the respective line in Table II, application no. 8, columns 5 or 8, or its homologs or fragments, by adding one or more exogenous inducing factors to the non-human organism or parts thereof; and/or
(f) expressing a transgenic gene encoding a protein conferring the increased expression of a FCRP, e.g. a polypeptide encoded by the nucleic acid molecule of the present invention or a polypeptide of the present invention, having herein-mentioned activity selected from the group consisting of said activities mentioned in (a) and conferring a linoleic acid increasing activity, respectively, e.g. of a polypeptide having the activity of a protein as indicated in the respective line in Table II, application no. 8, columns 5 or 8, or its homologs or fragments, and/or
(g) increasing the copy number of a gene conferring the increased expression of a nucleic acid molecule encoding a FCRP, e.g. a polypeptide encoded by the nucleic acid molecule of the invention or the polypeptide of the invention having herein-mentioned activity selected from the group consisting of said activities mentioned in (a) and conferring a linoleic acid increasing activity, respectively, e.g. of a polypeptide having the activity of a protein as indicated in the respective line in Table II, application no. 8, columns 5 or 8, or its homologs or fragments; and/or
(h) increasing the expression of the endogenous gene encoding the FCRP, polypeptide of the invention, e.g. a polypeptide encoded by the nucleic acid molecule of the invention or the polypeptide of the invention having herein-mentioned activity selected from the group consisting of said activities mentioned in (a) and conferring a linoleic acid; increasing activity, respectively, e.g. of a polypeptide having the activity of a protein as indicated in the respective line in Table II, application no. 8, columns 5 or 8, or its homologs or fragments, by adding positive expression or removing negative expression elements, e.g. homologous recombination can be used to either introduce positive regulatory elements like for plants the 35S enhancer into the promoter or to remove repressor elements form regulatory regions. Further gene conversion methods can be used to disrupt repressor elements or to enhance to activity of positive elements. Positive elements can be randomly introduced in plants by T-DNA or transposon mutagenesis and lines can be identified in which the positive elements have be integrated near to a gene of the invention, the expression of which is thereby enhanced; and/or
(i) modulating growth conditions of the non-human organism in such a manner, that the expression or activity of the gene encoding the FCRP, e.g. a protein of the invention or the protein itself is enhanced, for example microorganisms or plants can be grown for example under a higher temperature regime leading to an enhanced expression of heat shock proteins, which can lead to an enhanced linoleic acid production, respectively; and/or
(j) selecting of non-human organisms with especially high activity of the proteins of the invention from natural or from mutagenized resources and breeding them into the target organisms, e.g. the elite crops; and/or
(k) directing a protein encoded by the nucleic acid molecule of the invention or of the polypeptide of the present invention having herein-mentioned activity selected from the group consisting of said activities mentioned in (a) and conferring a linoleic acid increasing activity, respectively, e.g. of a polypeptide having the activity of a protein as indicated in the respective line in Table II, application no. 8, columns 5 or 8, or its homologs or fragments, if for the corresponding nucleic acid molecule in the respective line in column 6 of Table I the term “plastidic” is indicated, to the plastids by the addition of a plastidial targeting sequence or if for the corresponding nucleic acid molecule in the respective line in column 6 of Table I the term “mitochondrial” is indicated, to the mitochondria by the addition of a mitochondrial targeting sequence; and/or
(l) generating the expression of a protein encoded by the nucleic acid molecule of the invention or of the polypeptide of the present invention having herein-mentioned activity selected from the group consisting of said activities mentioned in (a) and conferring a linoleic acid increasing activity, respectively, e.g. of a polypeptide having the activity of a protein as indicated in the respective line in Table II, application no. 8, columns 5 or 8, or its homologs or fragments, if for the corresponding nucleic acid molecule in the respective line in column 6 of Table I the term “plastidic” or “mitochondrial” is indicated, in these organelles by the stable or transient transformation, advantageously stable transformation, of organelles, preferably plastids or mitochondria, with an inventive nucleic acid sequence preferably in form of an expression cassette containing said sequence leading to the expression of the nucleic acids or polypeptides of the invention in the respective organelle; and/or
(m) generating the expression of a protein encoded by the nucleic acid molecule of the invention or of the polypeptide of the present invention having herein-mentioned activity selected from the group consisting of said activities mentioned in (a) and conferring a linoleic acid increasing activity, respectively, e.g. of a polypeptide having the activity of a protein as indicated in the respective line in Table II, application no. 8, columns 5 or 8, or its homologs or fragments, if for the corresponding nucleic acid molecule in the respective line in column 6 of Table I the term “plastidic” or “mitochondrial” is indicated, in these organelles by integration of a nucleic acid of the invention into the genome of the respective organelle under control of preferable a promoter selective for the respective organelle.

Preferably, said mRNA is the coding region of the nucleic acid molecule of the present invention and/or the protein conferring the increased expression of a protein encoded by the coding region of the nucleic acid molecule of the present invention alone or linked to a transit nucleic acid sequence or transit peptide encoding nucleic acid sequence or the polypeptide having the herein mentioned activity, e.g. conferring the generation of or increase of linoleic acid, respectively, after increasing the expression or activity of the encoded polypeptide, non-targeted or in organelles such as plastids and/or mitochondria, preferably plastids, or having the activity of a polypeptide having an activity as the protein as shown in the respective line in Table II, application no. 8, column 3, or its homologs. Preferably the increase of linoleic acid, respectively, takes place non-targeted or in plastids and/or mitochondria, preferably non-targeted or in plastids.

In general, the amount of mRNA or polypeptide in a cell or a compartment of a non-human organism correlates with the amount of encoded protein and thus with the overall activity of the encoded protein in said volume. Said correlation is not always linear, the activity in the volume is dependent on the stability of the molecules, the degradation of the molecules or the presence of activating or inhibiting co-factors. Further, product and educt inhibitions of enzymes are well known and described in Textbooks, e.g. Stryer, Biochemistry or Zinser et al. “Enzyminhibitoren”/Enzyme inhibitors”.

The activity of the above-mentioned proteins and/or polypeptide encoded by the nucleic acid molecule of the present invention can be increased in various ways. For example, the activity in an organism or in a part thereof, like a cell or in an organelle of the cell, is increased for example via targeting of the nucleic acid sequence or the encoded gene product to an organelle such as plastids or mitochondria, preferentially to plastids, and/or increasing the gene product number, e.g. by increasing the expression rate, like introducing a stronger promoter, or by increasing the stability of the mRNA expressed, thus increasing the translation rate, and/or increasing the stability of the gene product, thus reducing the proteins decayed. Further, the activity or turnover of enzymes can be influenced in such a way that a reduction or increase of the reaction rate or a modification (reduction or increase) of the affinity to the substrate results, is reached. A mutation in the catalytic centre of an polypeptide of the invention, e.g. as enzyme, can modulate the turn over rate of the enzyme, e.g. an exchange of an amino acid in the catalytic center can lead to an increased activity of the enzyme, or the deletion of regulator binding sites can reduce a negative regulation like a feedback inhibition (or a substrate inhibition, if the substrate level is also increased). The specific activity of an enzyme of the present invention can be increased such that the turn over rate is increased or the binding of a co-factor is improved. Improving the stability of the encoding mRNA or the protein can also increase the activity of a gene product. The stimulation of the activity is also under the scope of the term “increased activity”.

Moreover, the regulation of the above-mentioned nucleic acid sequences may be modified so that gene expression is increased. This can be achieved advantageously by means of heterologous regulatory sequences or by modifying, for example mutating, the natural regulatory sequences which are present. The advantageous methods may also be combined with each other.

In general, an activity of a gene product in a non-human organism or part thereof, in particular in a plant cell or organelle of a plant cell, a plant, or a plant or a part thereof or in a microorganism can be increased by increasing the amount of the specific encoding mRNA or the corresponding protein in said organism or part thereof. “Amount of protein or mRNA” is understood as meaning the molecule number of polypeptides or mRNA molecules in an organism, a tissue, a cell or a cell compartment. “Increase” in the amount of a protein means the quantitative increase of the molecule number of said protein in a non-human organism, a cell or a cell compartment, such as an organelle like a plastid or mitochondrion, or part thereof for example by one of the methods described herein below—in comparison to a wild type, control or reference.

The increase in molecule number amounts preferably to at least 1%, 5%, 10%, 20%, 30%, 50%, 70%, 100%, 150%, 200% or more. However, a de novo expression is also regarded as subject of the present invention.

A modification, i.e. an increase, can be caused by endogenous or exogenous factors. For example, an increase in activity in a non-human organism or a part thereof can be caused by adding a gene product or a precursor or an activator or an agonist to the media or nutrition or can be caused by introducing said subjects into an organism, transient or stable. Such an increase can be reached by the introduction of the inventive nucleic acid sequence or the encoded protein into an organism, transient or stable. Furthermore such an increase can be reached by the introduction of the inventive nucleic acid sequence, preferably the coding region thereof, or the encoded protein in the correct cell compartment, for example into organelles, such as plastids or mitochondria, either by transformation and/or targeting.

In one embodiment the increase in the amount of the fine chemical in the non-human organism or a part thereof, e.g. in a cell, an organ, an organelle etc., is achieved by increasing the endogenous level of the polypeptide of the invention. Accordingly, in an embodiment of the present invention, the present invention relates to a process wherein the gene copy number of a gene encoding the polynucleotide or nucleic acid molecule of the invention is increased. Further, the endogenous level of the polypeptide of the invention can for example be increased by modifying the transcriptional or translational regulation of the polypeptide.

In one embodiment the amount of the fine chemical in the non-human organism or part thereof can be increase by targeted or random mutagenesis of the endogenous genes of the invention. For example homologous recombination can be used to either introduce positive regulatory elements like for plants the 35S enhancer into the promoter or to remove repressor elements form regulatory regions. In addition gene conversion like methods described by Kochevenko and Willmitzer (Plant Physiol. 132 (1), 174 (2003)) and citations therein can be used to disrupt repressor elements or to enhance to activity of positive regulatory elements.

Furthermore positive elements can be randomly introduced in (plant) genomes by T-DNA or transposon mutagenesis and lines can be screened for, in which the positive elements has be integrated near to a gene of the invention, the expression of which is thereby enhanced. The activation of plant genes by random integrations of enhancer elements has been described by Hayashi et al. (Science 258,1350 (1992)) or Weigel et al. (Plant Physiol. 122, 1003 (2000)) and others citated therein. Reverse genetic strategies to identify insertions (which eventually carrying the activation elements) near in genes of interest have been described for various cases e.g. Krysan et al. (Plant Cell 11, 2283 (1999)); Sessions et al. (Plant Cell 14, 2985 (2002)); Young et al. (Plant Physiol. 125, 513 (2001)); Koprek et al. (Plant J. 24, 253 (2000)); Jeon et al.(Plant J. 22, 561 (2000)); Tissier et al., (Plant Cell 11, 1841 (1999)); Speulmann et al. (Plant Cell 11, 1853 (1999)). Briefly, material from all plants of a large T-DNA or transposon mutagenized plant population is harvested and genomic DNA prepared. Then the genomic DNA is pooled following specific architectures as described for example in Krysan et al. (Plant Cell 11, 2283 (1999)). Pools of genomics DNAs are then screened by specific multiplex PCR reactions detecting the combination of the insertional mutagen (e.g. T-DNA or Transposon) and the gene of interest. Therefore PCR reactions are run on the DNA pools with specific combinations of T-DNA or transposon border primers and gene specific primers. General rules for primer design can again be taken from Krysan et al. (Plant Cell 11, 2283 (1999)). Rescreening of lower levels DNA pools lead to the identification of individual plants in which the gene of interest is disrupted by the insertional mutagen.

The enhancement of positive regulatory elements or the disruption or weaking of negative regulatory elements can also be achieved through common mutagenesis techniques: The production of chemically or radiation mutated populations is a common technique and known to the skilled worker. Methods for plants are described by Koorneef et al. 1982 and the citations therein and by Lightner and Caspar in “Methods in Molecular Biology” Vol. 82. These techniques usually induce point mutations that can be identified in any known gene using methods such as TILLING (Colbert et al., Plant Physiol.126 (2), 480 (2001)). One can also envisage to introduce nucleic acids sequences, encoding organelle- ,such as plastidal- or mitochondrial-targeting signals, like for example present in Table a or Table b, respectively, by homologous recombination or other methods of site specific integration, into the genome in that way, that an endogenous gene is functionally fused to the targeting sequence and the protein is redirected to the plastids or mitochondria, respectively. Eventually the integration can also occur randomly and the desired fusion event is selected for.

Accordingly, the expression level can be increased if the endogenous genes encoding a polypeptide conferring an increased expression of the polypeptide of the present invention, in particular genes comprising the nucleic acid molecule of the present invention, are modified via homologous recombination, Tilling approaches or gene conversion. It is also possible to add as mentioned herein targeting sequences to the inventive nucleic acid sequences.

Regulatory sequences, preferably in addition to a targeting sequence or part thereof (if present), can be operatively linked to the coding region of an endogenous nucleic acid and control its transcription and translation or the stability or decay of the encoding mRNA or the expressed protein. In order to modify and control the expression, promoter, UTRs, splicing sites, processing signals, polyadenylation sites, terminators, enhancers, repressors, post transcriptional or posttranslational modification sites can be changed, added or amended. For example, the activation of plant genes by random integrations of enhancer elements has been described by Hayashi et al. (Science 258, 1350 (1992)) or Weigel et al. (Plant Physiol. 122, 1003 (2000)) and others citated therein. For example, the expression level of the endogenous protein can be modulated by replacing the endogenous promoter with a stronger transgenic promoter or by replacing the endogenous 3′UTR with a 3′UTR, which provides more stability without amending the coding region. Further, the transcriptional regulation can be modulated by introduction of an artificial transcription factor as described in the examples. Alternative promoters, terminators and UTR are described below.

The activation of an endogenous polypeptide having above-mentioned activity, e.g. having the activity of a protein as shown in the respective line in Table II, application no. 8, column 3 or of the polypeptide of the invention, e.g. conferring the increase of the fine chemical linoleic acid, respectively, by increase of expression or activity in the cytoplasm, and/or in the cytosol, and/or in an organelle, such as plastids or mitochondria, can also be increased by introducing a synthetic transcription factor, which binds close to the coding region of the gene encoding the protein as shown in the respective line in Table II, application no. 8, column 5 or 8, or homologs or fragments thereof, and activates its transcription. A chimeric zinc finger protein can be constructed, which comprises a specific DNA-binding domain and an activation domain as e.g. the VP16 domain of Herpes Simplex virus. The specific binding domain can bind to the regulatory region of the gene encoding the protein as shown in the respective line in Table II, application no. 8, column 5 or 8, or homologs or fragments thereof. The expression of the chimeric transcription factor in a non-human organism, in particular in a plant, leads to a specific expression of the protein as shown in the respective line in Table II, application no. 8, column 5 or 8, or homologs or fragments thereof, see e.g. in WO01/52620, Oriz, Proc. Natl. Acad. Sci. USA 99, 13290 (2002), or Guan, Proc. Natl. Acad. Sci. USA 99, 13296 (2002).

In one further embodiment of the process according to the invention, organisms are used in which one of the above-mentioned genes, or one of the above-mentioned nucleic acids, is mutated in a way that the activity of the encoded gene products is less influenced by cellular factors, or not at all, in comparison with the unmutated proteins. For example, well known regulation mechanisms of enzymic activity are substrate inhibition or feed back regulation mechanisms. Ways and techniques for the introduction of substitution, deletions and additions of one or more bases, nucleotides or amino acids of a corresponding sequence are described herein below in the corresponding paragraphs and the references listed there, e.g. in Sambrook et al., Molecular Cloning, Cold Spring Habour, N.Y., 1989. The person skilled in the art will be able to identify regulation domains and binding sites of regulators by comparing the sequence of the nucleic acid molecule of the present invention or the expression product thereof with the state of the art by computer software means which comprise algorithms for the identifying of binding sites and regulation domains or by introducing into a nucleic acid molecule or in a protein systematically mutations and assaying for those mutations which will lead to an increased specify activity or an increased activity per volume, in particular per cell.

It can therefore be advantageous to express in a non-human organism a nucleic acid molecule of the invention or a polypeptide of the invention derived from a evolutionary distantly related organism, as e.g. using a prokaryotic gene in a eukaryotic host, as in these cases the regulation mechanism of the host cell may not weaken the activity (cellular or specific) of the gene or its expression product.

In another embodiment it can be advantageous to express in a non-human organism a nucleic acid molecule of the invention or a polypeptide of the invention in a compartment by the use of a respective targeting sequence, in which it is naturally not expressed, as in these cases the regulation mechanism of the host cell may not weaken the activity (cellular or specific) of the gene or its expression product.

The mutation is introduced in such a way that the production of the fine chemical is not adversely affected by e.g. undesired mutations.

Less influence on the regulation of a gene or its gene product is understood as meaning a reduced regulation of the enzymatic or biological activity leading to an increased specific or cellular activity of the gene or its product. An increase of the enzymatic activity is understood as meaning an enzymatic or biological activity, which is increased by at least 10%, 20%, 30%, 40%, 50%, 60% or 70% in comparison with the starting organism. This leads to an increased productivity of the desired fine chemical(s).

Owing to the introduction of a gene or a plurality of genes conferring the expression of the nucleic acid molecule of the invention or the polypeptide of the invention, for example the nucleic acid construct mentioned below, or encoding the protein as shown in the respective line in Table II, application no. 8, column 5 or 8, or homologs or fragments thereof, into a non-human organism alone or in combination with other genes, it is possible not only to increase the biosynthetic flux towards the end product, but also to increase, modify or create de novo an advantageous, preferably novel metabolites composition in the non-human organism, e.g. an advantageous composition comprising a higher content of (from a viewpoint of nutritional physiology limited) linoleic acid and if desired other fatty acids, and/or other metabolites, in free or bound form.

In another embodiment the composition comprises further higher amounts of metabolites positively affecting or lower amounts of metabolites negatively affecting the nutrition or health of animals or humans provided with said compositions or non-human organisms of the invention or parts thereof. Likewise, the number or activity of further genes which are required for the import or export of nutrients or metabolites, including amino acids, fatty acids, vitamins etc. or its precursors, required for the cell's biosynthesis of the fine chemical may be increased so that the concentration of necessary or relevant precursors, cofactors or intermediates within the cell(s) or within the corresponding storage compartments is increased. Owing to the increased or novel generated activity of the polypeptide of the invention or owing to the increased number of nucleic acid sequences of the invention and/or to the modulation of further genes which are involved in the biosynthesis of the fine chemical, e.g. by increasing the activity of enzymes synthesizing precursors or by destroying the activity of one or more genes which are involved in the breakdown of the fine chemical, it is possible to increase the yield, production and/or production efficiency of the fine chemical in the host organism, such as plants or microorganisms.

Accordingly, in one embodiment, the process according to the invention relates to a process, which comprises:
(a) providing a non-human organism, preferably a microorganism, a plant cell, a plant tissue, a plant or a part thereof, more preferably a microorganism, a plant tissue, a plant or a part thereof;
(b) increasing the activity of a protein as shown in the respective line in Table II, application no. 8, column 3 or of a polypeptide being encoded by the respective nucleic acid molecule of the present invention and described below, e.g. conferring an increase of the fine chemical linoleic acid, respectively, in the non-human organism, preferably in the microorganism, the plant cell, the plant tissue, the plant or a part thereof, more preferably a microorganism, a plant tissue, a plant or a part thereof, especially cytoplasmic or in an organelle, like plastids or mitochondria,
(c) growing the non-human organism, preferably the microorganism, the plant cell, the plant tissue, the plant or a part thereof, under conditions which permit the production of said fine chemical in the non-human organism, preferably the microorganism, the plant cell, the plant tissue, the plant or a part thereof; and
(d) if desired, recovering, optionally isolating, said fine chemical, in free and/or bound form, and, optionally further free and/or bound fatty acids, and/or other metabolites synthesized by the non-human organism, the microorganism, the plant cell, the plant tissue, the plant or a part thereof.

After the above-described increasing (which as defined above also encompasses the generating of an activity in a non-human organism, i.e. a de novo activity), for example after the introduction and the expression of the nucleic acid molecules of the invention or described in the methods or processes according to the invention, the organism according to the invention, advantageously, a microorganism, a non-human animal, a plant, plant or animal tissue or plant or animal cell, is grown and subsequently harvested.

Suitable non-human organisms or host organisms (transgenic organism) for the nucleic acid molecule used according to the invention and for the inventive process, the nucleic acid construct or the vector (both as described below) are, in principle, all organisms which are capable of synthesizing the fine chemical, and which are suitable for the activation, introduction or stimulation of recombinant genes. Examples which may be mentioned are transgenic plants, transgenic microorganisms such as fungi, bacteria, yeasts, alga or diatom preferably alga. Preferred organisms are those which are naturally capable of synthesizing the fine chemical in substantial amounts, like fungi, yeasts, bacteria or plants preferably alga and plants.

In the event that the transgenic non-human organism is a microorganism, such as a eukaryotic organism, for example a fungus, an alga, diatom or a yeast, in particular a fungus, alga, diatom or yeast selected from the families Tuberculariaceae, Adelotheciaceae, Dinophyceae, Ditrichaceae or Prasinophyceae. Preferred non-human organisms are microorganisms such as green algae or plants. After the growing phase, the organisms can be harvested.

The non-human organism, in particular the microorganism, plant or part thereof, is advantageously grown in such a way that it is not only possible to recover, if desired to isolate the free or bound fine chemical or the free and bound fine chemical but as option it is also possible to produce, recover and, if desired to isolate, other free or/and bound fatty acids and/or other metabolites.

The organisms are harvested in a manner known per se and appropriate for the particular organism. Microorganisms such as bacteria, mosses, yeasts and fungi or plant cells which are cultured in liquid media by fermentation may be removed, for example, by centrifugation, decanting or filtration. Plants are grown on solid media in a manner known per se and harvested accordingly.

Fatty Acids are isolated from the harvested biomass in a manner known per se, for example by extraction and, where appropriate, further chemical or physical purification processes such as, for example, precipitation methods, crystallography, thermal separation methods such as rectification methods or physical separation methods such as, for example, chromatography. Especially extraction methods using plant materials are described by Cahoon et al. (Proc. Natl. Acad. Sci. USA 96 (22), 12935 (1999)) or Browse et al., Analytic Biochemistry 152,141 (1986)).

Preferred microorganisms are selected from the group consisting of Charophyceae such as the genera Chara, Nitella e.g. the species Chara globularis, Chara vulgaris, Nitella flexilis, Chlorophyceae such as the genera Acrosiphonia, Spongomorpha, Urospora, Bryopsis, Pseudobryopsis, Trichosolen, Dichotomosiphon, Caulerpa, Rhipilia, Blastophysa, Avrainvillea, Chlorodesmis, Codium, Espera, Halicystis, Halimeda, Penicillus, Pseudocodium, Rhipiliopsis, Rhipocephalus, Tydemania, Udotea, Derbesia, Acrochaete, Aphanochaete, Bolbocoleon, Chaetobolus, Chaetonema, Chaetophora, Chlorotylium, Desmococcus, Draparnaldia, Draparnaldiopsis, Ectochaete, Endophyton, Entocladia, Epicladia, Internoretia, Microthamnion, Ochlochaete, Phaeophila, Pilinella, Pringsheimiella, Protoderma, Pseudendoclonium, Pseudodictyon, Pseudopringsheimia, Pseudulvella, Schizomeris, Stigeoclonium, Thamniochaete, Ulvella, Pilinia, Tellamia, Helicodictyon, Actidesmium, Ankyra, Characium, Codiolum, Sykidion, Keratococcus, Prototheca, Bracteacoccus Chlorococcum, Excentrosphaera, Hormidium, Oophila, Schroederia, Tetraedron, Trebouxia Chlorosarcinopsis, Gomphonitzschia, Coccomyxa, Dactylothece, Diógenes, Disporá, Gloeocystis, Mycanthococcus, Ourococcus, Coelastrum, Dicranochaete, Botryococcus, Dictyosphaerium, Dimorphococcus, Chlorochytrium, Kentrosphaera, Phyllobium, Gomontia, Hormotila, Euastropsis, Hydrodictyon, Pectodictyon, Pediastrum, Sorastrum, Tetrapedia, Acanthosphaera, Echinosphaerella, Echinosphaeridium, Errerella, Gloeoactinium, Golenkeniopsis Golenkinia, Micractinium, Ankistrodesmus, Chlorella, Chodatella, Closteriopsis, Cryocystis, Dactylococcus, Dematractum, Eremosphaera, Eutetramorus, Franceia, Glaucocystis, Gloeotaenium, Kirchneriella, Lagerheimiella, Monoraphidium, Nannochloris, Nephrochlamys, Nephrocytium, Oocystis, Oonephris, Pachycladon, Palmellococcus, Planktosphaeria, Polyedriopsis, Pseudoraciborskia, Quadrigula, Radiococcus, Rochiscia, Scotiella, Selanastrum, Thorakochloris, Treubaria, Trochiscia, Westella, Zoochlorella, Ostreobium, Phyllosiphon, Protosiphon, Rhodochytrium, Actinastrum, Coronastrum, Crucigenia, Dictymocystis, Enallax, Scenedesmus, Selenastrum, Tetradesmus, Tetrallantos, Tetrastrum, Chlorosarcina, Anadyomene, Valoniopsis, Ventricaria, Basicladia, Chaetomorpha, Cladophora, Lola Pithophora Rhizoclonium Chaetosphaeridium, Conochaete, Coleochaete, Oligochaetophora, Polychaetophora, Cylindrocapsa, Gongrosira, Protococcus, Acetabularia, Batophora, Bornetella, Dasycladus, Halicoryne, Neomeris, Elakatothrix, Raphidonema, Microspora, Bulbochaete, Oedocladium, Oedogonium, Prasiola, Rosenvingiella, Schizogonium, Apjohnia, Chamaedoris, Cladophoropsis, Siphonocladus, Spongocladia, Boergesenia, Boodlea, Cystodictyon, Dictyosphaeria, Ernodesmis, Microdictyon, Struvea, Valonia, Sphaeroplea, Malleochloris, Stylosphaeridium, Gloeococcus, Palmella, Palmodictyon, Palmophyllum, Pseudospherocystis, Sphaerocystis, Urococcus, Apiocystis, Chaetopeltis, Gemellicystis, Paulschulzia, Phacomyxa, Pseudotetraspora, Schizochlamys, Tetraspora, Cephaleuros, Ctenocladus, Epibolium, Leptosira, Trentepohlia, Diplochaete, Monostroma, Binuclearia, Geminella, Klebsormidium, Planetonema, Radiofilum, Stichococcus, Ulothrix, Uronema, Blidingia, Capsosiphon, Chloropelta, Enteromorpha, Percursaria, Ulva, Ulvaria, Brachiomonas, Carteria, Chlainomonas, Chlamydomonas, Chlamydonephris, Chlorangium, Chlorogonium, Cyanidium, Fortiella, Glenomonas, Gloeomonas, Hyalogonium, Lobomonas Polytoma, Pyramichlamys, Scourfieldia, Smithsonimonas, Sphaerellopsis, Sphenochloris, Spirogonium, Collodictyon, Dunaliella, Haematococcus, Stephanosphaera, Coccomonas, Dysmorphococcus, Phacotus, Pteromonas, Thoracomonas, Wislouchiella, Mascherina, Pyrobotrys, Spondylomorum, Eudorina, Gonium, Oltmannsiella, Pandorina, Platydorina, Pleodorina, Stephanoon, Volvox, Volvulina, Actinotaenium, Arthrodesmus, Bambusina Closterium, Cosmarium, Desmidium, Euastrum, Groenbladia, Hyalotheca, Micrasterias, Penium, Phymatodocis, Pleurotaenium, Sphaerozosma, Spinoclosterium, Spinocosmarium, Spondylosium, Staurastrum, Tetmemorus, Triploceras, Xanthidium, Cylindrocystis, Genicularia, Gonatozygon, Mesotaenium, Netrium, Roya, Spirotaenia, Cosmocladium, Debarya, Docidium, Euastridium, Hallasia, Mougeotia, Mougeotiopsis, Sirogonium, Spirogyra, Staurodesmus, Teilingia, Zygnema, Zygogonium, e.g. the species Caulerpa taxifolia, Prototheca wickerhamii, Ankistrodesmus falcatus, Chlorella ellipsoidea, Chlorella pyrenoidosa, Clorella sorokiniana, Chlorella vulgaris, Scenedesmus obliquus, Scenedesmus quadricauda, Selenastrum capricornutum, Selenastrum undecimnotata, Cladophora glomerata, Chlamydomonas eugametos, Chlamydomonas reinhardtii, Cyanidium caldarium, Dunaliella salina, Dunaliella tertiolecta, Euglena gracilis, Haematococcus pluvialis, Coniugatophyceae, Prasinophyceae Trebouxiophyceae, Ulvophyceae, Chlorodendraceae, Pedinomonadales, Halosphaeraceae, Pterospermataceae, Monomastigaceae, Pyramimonadaceae, Chlorodendraceae such as the genera Prasinocladus e.g. the species Prasinocladus ascus, Halosphaeraceae, Pedinomonadales, Pedinomonadaceae such as the genera Pedinomonas, Pterospermataceae such as the genera Pachysphaera, Pterosperma, Halosphaera, Pyramimonas, Bacillariophyceae, Chrysophyceae, Craspedophyceae, Euglenophyceae, Prymnesiophyceae, Phaeophyceae, Dinophyceae, Rhodophyceae, Xanthophyceae, Prasinophyceae such as the genera Nephroselmis, Prasinococcus, Scherffelia, Tetraselmis, Mantoniella, Ostreococcus e.g. the species Nephroselmis olivacea, Prasinococcus capsulatus, Scherffelia dubia, Tetraselmis chui, Tetraselmis suecica, Mantoniella squamata or Ostreococcus tauri.

Preferred plants are monocotyledonous or dicotyledonous plants, preferably selected from the group consisting of Anacardiaceae such as the genera Pistacia, Mangifera, Anacardium e.g. the species Pistacia vera [pistachios, Pistazie], Mangifer indica [Mango] or Anacardium occidentale [Cashew]; Asteraceae such as the genera Calendula, Carthamus, Centaurea, Cichorium, Cynara, Helianthus, Lactuca, Locusta, Tagetes, Valeriana e.g. the species Calendula officinalis [Marigold], Carthamus tinctorius [safflower], Centaurea cyanus [cornflower], Cichorium intybus [blue daisy], Cynara scolymus [Artichoke], Helianthus annus [sunflower], Lactuca sativa, Lactuca crispa, Lactuca esculenta, Lactuca scariola L. ssp. sativa, Lactuca scariola L. var. integrate, Lactuca scariola L. var. integrifolia, Lactuca sativa subsp. romana, Locusta communis, Valeriana locusta [lettuce], Tagetes lucida, Tagetes erecta or Tagetes tenuifolia [Marigold]; Apiaceae such as the genera Daucus e.g. the species Daucus carota [carrot]; Betulaceae such as the genera Corylus e.g. the species Corylus avellana or Corylus colurna [hazelnut]; Boraginaceae such as the genera Borago e.g. the species Borago officinalis [borage]; Brassicaceae such as the genera Brassica, Melanosinapis, Sinapis, Arabadopsis e.g. the species Brassica napus, Brassica rapa ssp. [canola, oilseed rape, turnip rape], Sinapis arvensis Brassica juncea, Brassica juncea var. juncea, Brassica juncea var. crispifolia, Brassica juncea var. foliosa, Brassica nigra, Brassica sinapioides, Melanosinapis communis [mustard], Brassica oleracea [fodder beet] or Arabidopsis thaliana; Bromeliaceae such as the genera Anana, Bromelia e.g. the species Anana comosus, Ananas ananas or Bromelia comosa [pineapple]; Caricaceae such as the genera Carica e.g. the species Carica papaya [papaya]; Cannabaceae such as the genera Cannabis e.g. the species Cannabis sative [hemp], Convolvulaceae such as the genera Ipomea, Convolvulus e.g. the species Ipomoea batatus, Ipomoea pandurata, Convolvulus batatas, Convolvulus tiliaceus, Ipomoea fastigiata, Ipomoea tiliacea, Ipomoea triloba or Convolvulus panduratus [sweet potato, Man of the Earth, wild potato], Chenopodiaceae such as the genera Beta, i.e. the species Beta vulgaris, Beta vulgaris var. altissima, Beta vulgaris var. Vulgaris, Beta maritima, Beta vulgaris var. perennis, Beta vulgaris var. conditiva or Beta vulgaris var. esculenta [sugar beet]; Cucurbitaceae such as the genera Cucubita e.g. the species Cucurbita maxima, Cucurbita mixta, Cucurbita pepo or Cucurbita moschata [pumpkin, squash]; Elaeagnaceae such as the genera Elaeagnus e.g. the species Olea europaea [olive]; Ericaceae such as the genera Kalmia e.g. the species Kalmia latifolia, Kalmia angustifolia, Kalmia microphylla, Kalmia polifolia, Kalmia occidentalis, Cistus chamaerhodendros or Kalmia lucida [American laurel, broad-leafed laurel, calico bush, spoon wood, sheep laurel, alpine laurel, bog laurel, western bog-laurel, swamp-laurel]; Euphorbiaceae such as the genera Manihot, Janipha, Jatropha, Ricinus e.g. the species Manihot utilissima, Janipha manihot, Jatropha manihot, Manihot aipil, Manihot dulcis, Manihot manihot, Manihot melanobasis, Manihot esculenta [manihot, arrowroot, tapioca, cassava] or Ricinus communis [castor bean, Castor Oil Bush, Castor Oil Plant, Palma Christi, Wonder Tree]; Fabaceae such as the genera Pisum, Albizia, Cathormion, Feuillea, Inga, Pithecolobium, Acacia, Mimosa, Medicajo, Glycine, Dolichos, Phaseolus, Soja e.g. the species Pisum sativum, Pisum arvense, Pisum humile [pea], Albizia berteriana, Albizia julibrissin, Albizia lebbeck, Acacia berteriana, Acacia littoralis, Albizia berteriana, Albizzia berteriana, Cathormion berteriana, Feuillea berteriana, Inga fragrans, Pithecellobium berterianum, Pithecellobium fragrans, Pithecolobium berterianum, Pseudalbizzia berteriana, Acacia julibrissin, Acacia nemu, Albizia nemu, Feuilleea julibrissin, Mimosa julibrissin, Mimosa speciosa, Sericanrda julibrissin, Acacia lebbeck, Acacia macrophylla, Albizia lebbek, Feuilleea lebbeck, Mimosa lebbeck, Mimosa speciosa [bastard logwood, silk tree, East Indian Walnut], Medicago sativa, Medicago falcata, Medicago varia [alfalfa], Glycine max [soybean], Dolichos soja, Glycine gracilis, Glycine hispida, Phaseolus max or Soja hispida; Geraniaceae such as the genera Pelargonium, Cocos, Oleum e.g. the species Cocos nucifera, Pelargonium grossularioides or Oleum cocois [coconut]; Gramineae such as the genera Saccharum e.g. the species Saccharum officinarum; Juglandaceae such as the genera Juglans, Wallia e.g. the species Juglans regia, Juglans ailanthifolia, Juglans sieboldiana, Juglans cinerea, Wallia cinerea, Juglans bixbyi, Juglans californica, Juglans hindsii, Juglans intermedia, Juglans jamaicensis, Juglans major, Juglans microcarpa, Juglans nigra or Wallia nigra [walnut, black walnut, common walnut, persian walnut, white walnut, butternut, black walnut]; Lauraceae such as the genera Persea, Laurus e.g. the species laurel Laurus nobilis [bay, laurel, bay laurel, sweet bay], Persea americana Persea americana, Persea gratissima or Persea persea [avocado]; Leguminosae such as the genera Arachis e.g. the species Arachis hypogaea [peanut]; Linaceae such as the genera Linum, Adenolinum e.g. the species Linum usitatissimum, Linum humile, Linum austriacum, Linum bienne, Linum angustifolium, Linum catharticum, Linum flavum, Linum grandiflorum, Adenolinum grandiflorum, Linum lewisii, Linum narbonense, Linum perenne, Linum perenne var. lewisii, Linum pratense or Linum trigynum [flax, linseed]; Lythrarieae such as the genera Punica e.g. the species Punica granatum [pomegranate]; Malvaceae such as the genera Gossypium e.g. the species Gossypium hirsutum, Gossypium arboreum, Gossypium barbadense, Gossypium herbaceum or Gossypium thurberi [cotton]; Musaceae such as the genera Musa e.g. the species Musa nana, Musa acuminata, Musa paradisiaca, Musa spp. [banana]; Onagraceae such as the genera Camissonia, Oenothera e.g. the species Oenothera biennis or Camissonia brevipes [primrose, evening primrose]; Palmae such as the genera Elacis e.g. the species Elaeis guineensis [oil plam]; Papaveraceae such as the genera Papaver e.g. the species Papaver orientale, Papaver rhoeas, Papaver dubium [poppy, oriental poppy, corn poppy, field poppy, shirley poppies, field poppy, long-headed poppy, long-pod poppy]; Pedaliaceae such as the genera Sesamum e.g. the species Sesamum indicum [sesame]; Piperaceae such as the genera Piper, Artanthe, Peperomia, Steffensia e.g. the species Piper aduncum, Piper amalago, Piper angustifolium, Piper auritum, Piper betel, Piper cubeba, Piper longum, Piper nigrum, Piper retrofractum, Artanthe adunca, Artanthe elongata, Peperomia elongata, Piper elongatum, Steffensia elongata. [Cayenne pepper, wild pepper]; Poaceae such as the genera Hordeum, Secale, Avena, Sorghum, Andropogon, Holcus, Panicum, Oryza, Zea, Triticum e.g. the species Hordeum vulgare, Hordeum jubatum, Hordeum murinum, Hordeum secalinum, Hordeum distichon Hordeum aegiceras, Hordeum hexastichon, Hordeum hexastichum, Hordeum irregulare, Hordeum sativum, Hordeum secalinum [barley, pearl barley, foxtail barley, wall barley, meadow barley], Secale cereale [rye], Avena sativa, Avena fatua, Avena byzantina, Avena fatua var. sativa, Avena hybrida [oat], Sorghum bicolor, Sorghum halepense, Sorghum saccharatum, Sorghum vulgare, Andropogon drummondii, Holcus bicolor, Holcus sorghum, Sorghum aethiopicum, Sorghum arundinaceum, Sorghum caffrorum, Sorghum cernuum, Sorghum dochna, Sorghum drummondii, Sorghum durra, Sorghum guineense, Sorghum lanceolatum, Sorghum nervosum, Sorghum saccharatum, Sorghum subglabrescens, Sorghum verticilliflorum, Sorghum vulgare, Holcus halepensis, Sorghum miliaceum millet, Panicum militaceum [Sorghum, millet], Oryza sativa, Oryza latifolia [rice], Zea mays [corn, maize] Triticum aestivum, Triticum durum, Triticum turgidum, Triticum hybernum, Triticum macha, Triticum sativum or Triticum vulgare [wheat, bread wheat, common wheat], Proteaceae such as the genera Macadamia e.g. the species Macadamia intergrifolia [macadamia]; Rubiaceae such as the genera Coffea e.g. the species Cofea spp., Coffea arabica, Coffea canephora or Coffea liberica [coffee]; Scrophulariaceae such as the genera Verbascum e.g. the species Verbascum blattaria, Verbascum chaixii, Verbascum densiflorum, Verbascum lagurus, Verbascum longifolium, Verbascum lychnitis, Verbascum nigrum, Verbascum olympicum, Verbascum phlomoides, Verbascum phoenicum, Verbascum pulverulentum or Verbascum thapsus [mullein, white moth mullein, nettle-leaved mullein, dense-flowered mullein, silver mullein, long-leaved mullein, white mullein, dark mullein, greek mullein, orange mullein, purple mullein, hoary mullein, great mullein]; Solanaceae such as the genera Capsicum, Nicotiana, Solanum, Lycopersicon e.g. the species Capsicum annuum, Capsicum annuum var. glabriusculum, Capsicum frutescens [pepper], Capsicum annuum [paprika], Nicotiana tabacum, Nicotiana alata, Nicotiana attenuata, Nicotiana glauca, Nicotiana langsdorffii, Nicotiana obtusifolia, Nicotiana quadrivalvis, Nicotiana repanda, Nicotiana rustica, Nicotiana sylvestris [tobacco], Solanum tuberosum [potato], Solanum melongena [egg-plant] (Lycopersicon esculentum, Lycopersicon lycopersicum, Lycopersicon pyriforme, Solanum integrifolium or Solanum lycopersicum [tomato]; Sterculiaceae such as the genera Theobroma e.g. the species Theobroma cacao [cacao]; Theaceae such as the genera Camellia e.g. the species Camellia sinensis) [tea].

Particular preferred plants are monocotyledonous or dicotyledonous plants selected from the group consisting of Asteraceae such as the genera Helianthus, Tagetes e.g. the species Helianthus annus [sunflower], Tagetes lucida, Tagetes erecta or Tagetes tenuifolia [Marigold]; Brassicaceae such as the genera Brassica, Arabadopsis e.g. the species Brassica napus, Brassica rapa ssp. [canola, oilseed rape, turnip rape] or Arabidopsis thaliana; Fabaceae such as the genera Glycine e.g. the species Glycine max [soybean] or Soja hispida; Linaceae such as the genera Linum e.g. the species Linum usitatissimum, [flax, linseed]; Poaceae such as the genera Hordeum, Secale, Avena, Sorghum, Oryza, Zea, Triticum e.g. the species Hordeum vulgare [barley], Secale cereale [rye], Avena sativa, Avena fatua, Avena byzantina, Avena fatua var. sativa, Avena hybrida [oat], Sorghum bicolor [Sorghum, millet], Oryza sativa, Oryza latifolia [rice], Zea mays [corn, maize], Triticum aestivum, Triticum durum, Triticum turgidum, Triticum hybernum, Triticum macha, Triticum sativum or Triticum vulgare [wheat, bread wheat, common wheat]; Solanaceae such as the genera Solanum, Lycopersicon e.g. the species Solanum tuberosum [potato], Lycopersicon esculentum, Lycopersicon lycopersicum, Lycopersicon pyriforme, Solanum integrifolium or Solanum lycopersicum [tomato]; Gramineae such as the genera Saccharum e.g. the species Saccharum officinarum or Helianthus annus [sunflower] or Chenopodiaceae such as the genera Beta, i.e. Beta vulgaris var. esculents [sugar beet].

All above-mentioned organisms can be used as host organisms and/or can be used as donor organism.

The invention is not limited to specific nucleic acids, specific polypeptides, specific cell types, specific host cells, specific conditions or specific methods etc. as such, but may vary and numerous modifications and variations therein will be apparent to those skilled in the art. It is also to be understood that the terminology used herein is for the purpose of describing specific embodiments only and is not intended to be limiting.

In another embodiment, the invention relates to a (isolated) nucleic acid molecule, which comprises a nucleic acid molecule selected from the group consisting of:
(a) a nucleic acid molecule encoding a polypeptide shown in Table II, application no. 8, column 5 or 8, preferably shown in Table II B, application no. 8, column 8, or a homolog or a fragment thereof;
(b) a nucleic acid molecule shown in Table I, application no. 8, column 5 or 8, preferably shown in Table I B, application no. 8, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 8, preferably in column 8 of Table II B, application no. 8;
(d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 8, preferably shown in column 8 of Table I B, application no. 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 8;
(i) a nucleic acid molecule encoding a polypeptide having the activity represented by a respective protein as depicted in column 5 of Table II, application no. 8;
(j) a nucleic acid molecule comprising a polynucleotide which is obtained by amplifying a cDNA library or a genomic library using the primers in Table III, application no. 8, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j) ;
or a nucleic acid molecule comprising a sequence which is complementary thereto.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 8.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II application no. 8 and confers a generation or an increase of the respective fine chemical in a non-human organism or a part thereof as compared to a corresponding wild-type non-human organism or part thereof.

In another embodiment, the invention relates to a (isolated) nucleic acid molecule, which comprises a nucleic acid molecule selected from the group consisting of:
(a) a nucleic acid molecule encoding a polypeptide shown in Table II, column 5 or 8, application no. 8, preferably shown in Table II A, application no. 8, in column 5 or in Table II A, application no. 8, column 8 or in Table II B, application no. 8, column 8, or a homolog or a fragment thereof;
(b) a nucleic acid molecule shown in Table I, application no. 8, column 5 or 8, preferably shown in Table I A, application no. 8, in column 5 or in Table I A, application no. 8, column 8 or in Table I B, application no. 8, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 8, preferably shown in Table II A, application no. 8, in column 5 or in Table II A, application no. 8, column 8 or in Table II B, application no. 8, column 8;
(d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 8, preferably shown in Table I A, application no. 8, in column 5 or in Table I A, application no. 8, column 8 or in Table I B column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 8;
(i) a nucleic acid molecule encoding a polypeptide having the activity represented by a respective protein as depicted in column 5 of Table II, application no. 8;
(j) a nucleic acid molecule comprising a polynucleotide which is obtained by amplifying a cDNA library or a genomic library using the primers in Table III, application no. 8, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt,500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto;
   whereby, preferably, the nucleic acid molecule according to (a), (b), (c), (d), (e), (f), (g), (h), (i), (j) or (k) distinguishes over the sequence depicted in Table I A and/or I B, application no. 8, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 8, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 8, column 5 or 8, or the coding region thereof, respectively. In a further embodiment the nucleic acid molecule does not encode the polypeptide sequence shown in Table II A and/or II B, application no. 8, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 8, column 5 or 8 and does not encode a protein of the sequence shown in Table II A and/or II B, application no. 8, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 8, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 8, column 5 or 8 but less than 100%, preferably less than 99.999%, 99.99%, 99.9%, 99%, 98%, 97%, 96% or 95% identical to the sequence shown in Table II A and/or II B, application no. 8, column 5 or 8.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 8.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II, application no. 8, and confers a generation or an increase of the respective fine chemical in a non-human organism or a part thereof as compared to a corresponding wild-type non-human organism or part thereof.

In one embodiment the invention relates to the aforementioned nucleic acid molecules, which can be isolated advantageously from yeast, fungi, viruses, algae, bacteria, for example from Charophyceae such as the genera Chara, Nitella e.g. the species Chara globularis, Chara vulgaris, Nitella flexilis, Chlorophyceae such as the genera Acrosiphonia, Spongomorpha, Urospora, Bryopsis, Pseudobryopsis, Trichosolen, Dichotomosiphon, Caulerpa, Rhipilia, Blastophysa, Avrainvillea, Chlorodesmis, Codium, Espera, Halicystis, Halimeda, Penicillus, Pseudocodium, Rhipiliopsis, Rhipocephalus, Tydemania, Udotea, Derbesia, Acrochaete, Aphanochaete, Bolbocoleon, Chaetobolus, Chaetonema, Chaetophora, Chlorotylium, Desmococcus, Draparnaldia, Draparnaldiopsis, Ectochaete, Endophyton, Entocladia, Epicladia, Internoretia, Microthamnion, Ochlochaete, Phaeophila, Pilinella, Pringsheimiella, Protoderma, Pseudendoclonium, Pseudodictyon, Pseudopringsheimia, Pseudulvella, Schizomeris, Stigeoclonium, Thamniochaete, Ulvella, Pilinia, Tellamia, Helicodictyon, Actidesmium, Ankyra, Characium, Codiolum, Sykidion, Keratococcus, Prototheca, Bracteacoccus Chlorococcum, Excentrosphaera, Hormidium, Oophila, Schroederia, Tetraedron, Trebouxia Chlorosarcinopsis, Gomphonitzschia, Coccomyxa, Dactylothece, Diógenes, Disporá, Gloeocystis, Mycanthococcus, Ourococcus, Coelastrum, Dicranochaete, Botryococcus, Dictyosphaerium, Dimorphococcus, Chlorochytrium, Kentrosphaera, Phyllobium, Gomontia, Hormotila, Euastropsis, Hydrodictyon, Pectodictyon, Pediastrum, Sorastrum, Tetrapedia, Acanthosphaera, Echinosphaerella, Echinosphaeridium, Errerella, Gloeoactinium, Golenkeniopsis Golenkinia, Micractinium, Ankistrodesmus, Chlorella, Chodatella, Closteriopsis, Cryocystis, Dactylococcus, Dematractum, Eremosphaera, Eutetramorus, Franceia, Glaucocystis, Gloeotaenium, Kirchneriella, Lagerheimiella, Monoraphidium, Nannochloris, Nephrochlamys, Nephrocytium, Oocystis, Oonephris, Pachycladon, Palmellococcus, Planktosphaeria, Polyedriopsis, Pseudoraciborskia, Quadrigula, Radiococcus, Rochiscia, Scotiella, Selanastrum, Thorakochloris, Treubaria, Trochiscia, Westella, Zoochlorella, Ostreobium, Phyllosiphon, Protosiphon, Rhodochytrium, Actinastrum, Coronastrum, Crucigenia, Dictymocystis, Enallax, Scenedesmus, Selenastrum, Tetradesmus, Tetrallantos, Tetrastrum, Chlorosarcina, Anadyomene, Valoniopsis, Ventricaria, Basicladia, Chaetomorpha, Cladophora, Lola Pithophora Rhizoclonium Chaetosphaeridium, Conochaete, Coleochaete, Oligochaetophora, Polychaetophora, Cylindrocapsa, Gongrosira, Protococcus, Acetabularia, Batophora, Bornetella, Dasycladus, Halicoryne, Neomeris, Elakatothrix, Raphidonema, Microspora, Bulbochaete, Oedocladium, Oedogonium, Prasiola, Rosenvingiella, Schizogonium, Apjohnia, Chamaedoris, Cladophoropsis, Siphonocladus, Spongocladia, Boergesenia, Boodlea, Cystodictyon, Dictyosphaeria, Ernodesmis, Microdictyon, Struvea, Valonia, Sphaeroplea, Malleochloris, Stylosphaeridium, Gloeococcus, Palmella, Palmodictyon, Palmophyllum, Pseudospherocystis, Sphaerocystis, Urococcus, Apiocystis, Chaetopeltis, Gemellicystis, Paulschulzia, Phacomyxa, Pseudotetraspora, Schizochlamys, Tetraspora, Cephaleuros, Ctenocladus, Epibolium, Leptosira, Trentepohlia, Diplochaete, Monostroma, Binuclearia, Geminella, Klebsormidium, Planetonema, Radiofilum, Stichococcus, Ulothrix, Uronema, Blidingia, Capsosiphon, Chloropelta, Enteromorpha, Percursaria, Ulva, Ulvaria, Brachiomonas, Carteria, Chlainomonas, Chlamydomonas, Chlamydonephris, Chlorangium, Chlorogonium, Cyanidium, Fortiella, Glenomonas, Gloeomonas, Hyalogonium, Lobomonas Polytoma, Pyramichlamys, Scourfieldia, Smithsonimonas, Sphaerellopsis, Sphenochloris, Spirogonium, Collodictyon, Dunaliella, Haematococcus, Stephanosphaera, Coccomonas, Dysmorphococcus, Phacotus, Pteromonas, Thoracomonas, Wislouchiella, Mascherina, Pyrobotrys, Spondylomorum, Eudorina, Gonium, Oltmannsiella, Pandorina, Platydorina, Pleodorina, Stephanoon, Volvox, Volvulina, Actinotaenium, Arthrodesmus, Bambusina Closterium, Cosmarium, Desmidium, Euastrum, Groenbladia, Hyalotheca, Micrasterias, Penium, Phymatodocis, Pleurotaenium, Sphaerozosma, Spinoclosterium, Spinocosmarium, Spondylosium, Staurastrum, Tetmemorus, Triploceras, Xanthidium, Cylindrocystis, Genicularia, Gonatozygon, Mesotaenium, Netrium, Roya, Spirotaenia, Cosmocladium, Debarya, Docidium, Euastridium, Hallasia, Mougeotia, Mougeotiopsis, Sirogonium, Spirogyra, Staurodesmus, Teilingia, Zygnema, Zygogonium, e.g. the species Caulerpa taxifolia, Prototheca wickerhamii, Ankistrodesmus falcatus, Chlorella ellipsoidea, Chlorella pyrenoidosa, Clorella sorokiniana, Chlorella vulgaris, Scenedesmus obliquus, Scenedesmus quadricauda, Selenastrum capricornutum, Selenastrum undecimnotata, Cladophora glomerata, Chlamydomonas eugametos, Chlamydomonas reinhardtii, Cyanidium caldarium, Dunaliella salina, Dunaliella tertiolecta, Euglena gracilis, Haematococcus pluvialis, Coniugatophyceae, Prasinophyceae Trebouxiophyceae, Ulvophyceae, Chlorodendraceae, Pedinomonadales, Halosphaeraceae, Pterospermataceae, Monomastigaceae, Pyramimonadaceae, Chlorodendraceae such as the genera Prasinocladus e.g. the species Prasinocladus ascus, Halosphaeraceae, Pedinomonadales, Pedinomonadaceae such as the genera Pedinomonas, Pterospermataceae such as the genera Pachysphaera, Pterosperma, Halosphaera, Pyramimonas, Bacillariophyceae, Chrysophyceae, Craspedophyceae, Euglenophyceae, Prymnesiophyceae, Phaeophyceae, Dinophyceae, Rhodophyceae, Xanthophyceae, Prasinophyceae such as the genera Nephroselmis, Prasinococcus, Scherffelia, Tetraselmis, Mantoniella, Ostreococcus e.g. the species Nephroselmis olivacea, Prasinococcus capsulatus, Scherffelia dubia, Tetraselmis chui, Tetraselmis suecica, Mantoniella squamata, Ostreococcus tauri, especially such as Acetobacter (subgen. Acetobacter) aceti; Acidithiobacillus ferrooxidans; Acinetobacter sp.; Actinobacillus sp; Aeromonas salmonicida; Agrobacterium tumefaciens; Aquifex aeolicus; Arcanobacterium pyogenes; Aster yellows phytoplasma; Bacillus sp.; Bifidobacterium sp.; Borrelia burgdorferi; Brevibacterium linens; Brucella melitensis; Buchnera sp.; Butyrivibrio fibrisolvens; Campylobacter jejuni; Caulobacter crescentus; Chlamydia sp.; Chlamydophila sp.; Chlorobium limicola; Citrobacter rodentium; Clostridium sp.; Comamonas testosteroni; Corynebacterium sp.; Coxiella burnetii; Deinococcus radiodurans; Dichelobacter nodosus; Edwardsiella ictaluri; Enterobacter sp.; Erysipelothrix rhusiopathiae; E. coli; Flavobacterium sp.; Francisella tularensis; Frankia sp. Cpl1; Fusobacterium nucleatum; Geobacillus stearothermophilus; Gluconobacter oxydans; Haemophilus sp.; Helicobacter pylori; Klebsiella pneumoniae; Lactobacillus sp.; Lactococcus lactis; Listeria sp.; Mannheimia haemolytica; Mesorhizobium loti; Methylophaga thalassica; Microcystis aeruginosa; Microscilla sp. PRE1; Moraxella sp. TA144; Mycobacterium sp.; Mycoplasma sp.; Neisseria sp.; Nitrosomonas sp.; Nostoc sp. PCC 7120; Novosphingobium aromaticivorans; Oenococcus oeni; Pantoea citrea; Pasteurella multocida; Pediococcus pentosaceus; Phormidium foveolarum; Phytoplasma sp.; Plectonema boryanum; Prevotella ruminicola; Propionibacterium sp.; Proteus vulgaris; Pseudomonas sp.; Ralstonia sp.; Rhizobium sp.; Rhodococcus equi; Rhodothermus marinus; Rickettsia sp.; Riemerella anatipestifer; Ruminococcus flavefaciens; Salmonella sp.; Selenomonas ruminantium; Serratia entomophila; Shigella sp.; Sinorhizobium meliloti; Staphylococcus sp.; Streptococcus sp.; Streptomyces sp.; Synechococcus sp.; Synechocystis sp. PCC 6803; Thermotoga maritima; Treponema sp.; Ureaplasma urealyticum; Vibrio cholerae; Vibrio parahaemolyticus; Xylella fastidiosa; Yersinia sp.; Zymomonas mobilis, preferably Salmonella sp. or E. coli, preferably from yeasts such as from the genera Saccharomyces, Pichia, Candida, Hansenula, Torulopsis or Schizosaccharomyces, or from plants, such as monocotyledonous or dicotyledonous plants, preferably selected from the group consisting of Anacardiaceae such as the genera Pistacia, Mangifera, Anacardium e.g. the species Pistacia vera [pistachios, Pistazie], Mangifer indica [Mango] or Anacardium occidentale [Cashew]; Asteraceae such as the genera Calendula, Carthamus, Centaurea, Cichorium, Cynara, Helianthus, Lactuca, Locusta, Tagetes, Valeriana e.g. the species Calendula officinalis [Marigold], Carthamus tinctorius [safflower], Centaurea cyanus [cornflower], Cichorium intybus [blue daisy], Cynara scolymus [Artichoke], Helianthus annus [sunflower], Lactuca sativa, Lactuca crispa, Lactuca esculenta, Lactuca scariola L. ssp. sativa, Lactuca scariola L. var. integrata, Lactuca scariola L. var. integrifolia, Lactuca sativa subsp. romana, Locusta communis, Valeriana locusta [lettuce], Tagetes lucida, Tagetes erecta or Tagetes tenuifolia [Marigold]; Apiaceae such as the genera Daucus e.g. the species Daucus carota [carrot]; Betulaceae such as the genera Corylus e.g. the species Corylus avellana or Corylus colurna [hazelnut]; Boraginaceae such as the genera Borago e.g. the species Borago officinalis [borage]; Brassicaceae such as the genera Brassica, Melanosinapis, Sinapis, Arabadopsis e.g. the species Brassica napus, Brassica rapa ssp. [canola, oilseed rape, turnip rape], Sinapis arvensis Brassica juncea, Brassica juncea var. juncea, Brassica juncea var. crispifolia, Brassica juncea var. foliosa, Brassica nigra, Brassica sinapioides, Melanosinapis communis [mustard], Brassica oleracea [fodder beet] or Arabidopsis thaliana; Bromeliaceae such as the genera Anana, Bromelia e.g. the species Anana comosus, Ananas ananas or Bromelia comosa [pineapple]; Caricaceae such as the genera Carica e.g. the species Carica papaya [papaya]; Cannabaceae such as the genera Cannabis e.g. the species Cannabis sative [hemp], Convolvulaceae such as the genera Ipomea, Convolvulus e.g. the species Ipomoea batatus, Ipomoea pandurata, Convolvulus batatas, Convolvulus tiliaceus, Ipomoea fastigiata, Ipomoea tiliacea, Ipomoea triloba or Convolvulus panduratus [sweet potato, Man of the Earth, wild potato], Chenopodiaceae such as the genera Beta, i.e. the species Beta vulgaris, Beta vulgaris var. altissima, Beta vulgaris var. Vulgaris, Beta maritima, Beta vulgaris var. perennis, Beta vulgaris var. conditiva or Beta vulgaris var. esculenta [sugar beet]; Cucurbitaceae such as the genera Cucubita e.g. the species Cucurbita maxima, Cucurbita mixta, Cucurbita pepo or Cucurbita moschata [pumpkin, squash]; Elaeagnaceae such as the genera Elaeagnus e.g. the species Olea europaea [olive]; Ericaceae such as the genera Kalmia e.g. the species Kalmia latifolia, Kalmia angustifolia, Kalmia microphylla, Kalmia polifolia, Kalmia occidentalis, Cistus chamaerhodendros or Kalmia lucida [American laurel, broad-leafed laurel, calico bush, spoon wood, sheep laurel, alpine laurel, bog laurel, western bog-laurel, swamp-laurel]; Euphorbiaceae such as the genera Manihot, Janipha, Jatropha, Ricinus e.g. the species Manihot utilissima, Janipha manihot, Jatropha manihot, Manihot aipil, Manihot dulcis, Manihot manihot, Manihot melanobasis, Manihot esculenta [manihot, arrowroot, tapioca, cassava] or Ricinus communis [castor bean, Castor Oil Bush, Castor Oil Plant, Palma Christi, Wonder Tree]; Fabaceae such as the genera Pisum, Albizia, Cathormion, Feuillea, Inga, Pithecolobium, Acacia, Mimosa, Medicajo, Glycine, Dolichos, Phaseolus, Soja e.g. the species Pisum sativum, Pisum arvense, Pisum humile [pea], Albizia berteriana, Albizia julibrissin, Albizia lebbeck, Acacia berteriana, Acacia littoralis, Albizia berteriana, Albizzia berteriana, Cathormion berteriana, Feuillea berteriana, Inga fragrans, Pithecellobium berterianum, Pithecellobium fragrans, Pithecolobium berterianum, Pseudalbizzia berteriana, Acacia julibrissin, Acacia nemu, Albizia nemu, Feuilleea julibrissin, Mimosa julibrissin, Mimosa speciosa, Sericanrda julibrissin, Acacia lebbeck, Acacia macrophylla, Albizia lebbek, Feuilleea lebbeck, Mimosa lebbeck, Mimosa speciosa [bastard logwood, silk tree, East Indian Walnut], Medicago sativa, Medicago falcata, Medicago varia [alfalfa], Glycine max [soybean], Dolichos soja, Glycine gracilis, Glycine hispida, Phaseolus max or Soja hispida; Geraniaceae such as the genera Pelargonium, Cocos, Oleum e.g. the species Cocos nucifera, Pelargonium grossularioides or Oleum cocois [coconut]; Gramineae such as the genera Saccharum e.g. the species Saccharum officinarum; Juglandaceae such as the genera Juglans, Wallia e.g. the species Juglans regia, Juglans ailanthifolia, Juglans sieboldiana, Juglans cinerea, Wallia cinerea, Juglans bixbyi, Juglans californica, Juglans hindsii, Juglans intermedia, Juglans jamaicensis, Juglans major, Juglans microcarpa, Juglans nigra or Wallia nigra [walnut, black walnut, common walnut, persian walnut, white walnut, butternut, black walnut]; Lauraceae such as the genera Persea, Laurus e.g. the species laurel Laurus nobilis [bay, laurel, bay laurel, sweet bay], Persea americana Persea americana, Persea gratissima or Persea persea [avocado]; Leguminosae such as the genera Arachis e.g. the species Arachis hypogaea [peanut]; Linaceae such as the genera Linum, Adenolinum e.g. the species Linum usitatissimum, Linum humile, Linum austriacum, Linum bienne, Linum angustifolium, Linum catharticum, Linum flavum, Linum grandiflorum, Adenolinum grandiflorum, Linum lewisii, Linum narbonense, Linum perenne, Linum perenne var. lewisii, Linum pratense or Linum trigynum [flax, linseed]; Lythrarieae such as the genera Punica e.g. the species Punica granatum [pomegranate]; Malvaceae such as the genera Gossypium e.g. the species Gossypium hirsutum, Gossypium arboreum, Gossypium barbadense, Gossypium herbaceum or Gossypium thurberi [cotton]; Musaceae such as the genera Musa e.g. the species Musa nana, Musa acuminate, Musa paradisiaca, Musa spp. [banana]; Onagraceae such as the genera Camissonia, Oenothera e.g. the species Oenothera biennis or Camissonia brevipes [primrose, evening primrose]; Palmae such as the genera Elacis e.g. the species Elaeis guineensis [oil plam]; Papaveraceae such as the genera Papaver e.g. the species Papaver orientale, Papaver rhoeas, Papaver dubium [poppy, oriental poppy, corn poppy, field poppy, shirley poppies, field poppy, longheaded poppy, long-pod poppy]; Pedaliaceae such as the genera Sesamum e.g. the species Sesamum indicum [sesame]; Piperaceae such as the genera Piper, Artanthe, Peperomia, Steffensia e.g. the species Piper aduncum, Piper amalago, Piper angustifolium, Piper auritum, Piper betel, Piper cubeba, Piper longum, Piper nigrum, Piper retrofractum, Artanthe adunca, Artanthe elongata, Peperomia elongata, Piper elongatum, Steffensia elongata. [Cayenne pepper, wild pepper]; Poaceae such as the genera Hordeum, Secale, Avena, Sorghum, Andropogon, Holcus, Panicum, Oryza, Zea, Triticum e.g. the species Hordeum vulgare, Hordeum jubatum, Hordeum murinum, Hordeum secalinum, Hordeum distichon Hordeum aegiceras, Hordeum hexastichon, Hordeum hexastichum, Hordeum irregulare, Hordeum sativum, Hordeum secalinum [barley, pearl barley, foxtail barley, wall barley, meadow barley], Secale cereale [rye], Avena sativa, Avena fatua, Avena byzantina, Avena fatua var. sativa, Avena hybrida [oat], Sorghum bicolor, Sorghum halepense, Sorghum saccharatum, Sorghum vulgare, Andropogon drummondii, Holcus bicolor, Holcus sorghum, Sorghum aethiopicum, Sorghum arundinaceum, Sorghum caffrorum, Sorghum cernuum, Sorghum dochna, Sorghum drummondii, Sorghum durra, Sorghum guineense, Sorghum lanceolatum, Sorghum nervosum, Sorghum saccharatum, Sorghum subglabrescens, Sorghum verticilliflorum, Sorghum vulgare, Holcus halepensis, Sorghum miliaceum millet, Panicum militaceum [Sorghum, millet], Oryza sativa, Oryza latifolia [rice], Zea mays [corn, maize] Triticum aestivum, Triticum durum, Triticum turgidum, Triticum hybernum, Triticum macha, Triticum sativum or Triticum vulgare [wheat, bread wheat, common wheat], Proteaceae such as the genera Macadamia e.g. the species Macadamia intergrifolia [macadamia]; Rubiaceae such as the genera Coffea e.g. the species Cofea spp., Coffea arabica, Coffea canephora or Coffea liberica [coffee]; Scrophulariaceae such as the genera Verbascum e.g. the species Verbascum blattaria, Verbascum chaixii, Verbascum densiflorum, Verbascum lagurus, Verbascum longifolium, Verbascum lychnitis, Verbascum nigrum, Verbascum olympicum, Verbascum phlomoides, Verbascum phoenicum, Verbascum pulverulentum or Verbascum thapsus [mullein, white moth mullein, nettle-leaved mullein, dense-flowered mullein, silver mullein, long-leaved mullein, white mullein, dark mullein, greek mullein, orange mullein, purple mullein, hoary mullein, great mullein]; Solanaceae such as the genera Capsicum, Nicotiana, Solanum, Lycopersicon e.g. the species Capsicum annuum, Capsicum annuum var. glabriusculum, Capsicum frutescens [pepper], Capsicum annuum [paprika], Nicotiana tabacum, Nicotiana alata, Nicotiana attenuata, Nicotiana glauca, Nicotiana langsdorffii, Nicotiana obtusifolia, Nicotiana quadrivalvis, Nicotiana repanda, Nicotiana rustica, Nicotiana sylvestris [tobacco], Solanum tuberosum [potato], Solanum melongena [egg-plant] (Lycopersicon esculentum, Lycopersicon lycopersicum, Lycopersicon pyriforme, Solanum integrifolium or Solanum lycopersicum [tomato]; Sterculiaceae such as the genera Theobroma e.g. the species Theobroma cacao [cacao]; Theaceae such as the genera Camellia e.g. the species Camellia sinensis) [tea]; especially A. thaliana, maize, wheat, rye, oat, triticale, rice, barley, soybean, peanut, cotton, borage, sunflower, sugar cane, sugar beet, linseed, primrose, rapeseed, canola and turnip rape, manihot, pepper, sunflower, tagetes, solanaceous plant such as potato, tobacco, eggplant and tomato, Vicia species, pea, alfalfa, bushy plants such as coffee, cacao, tea, Salix species, trees such as oil palm, coconut, perennial grass, such as ryegrass and fescue, and forage crops, such as alfalfa and clover and from spruce, pine or fir for example.

More preferably said nucleic acid molecules can be isolated from S. cerevisiae, E. coli, Azotobacter vinelandii, Thermus thermophilus or Synechocystis sp. or plants, preferably Arabidopsis thaliana, Brassica napus, Glycine max, Zea mays, Gossypium or Oryza sativa.

The FCRPs of the present invention are preferably produced by recombinant DNA techniques. For example, a nucleic acid molecule encoding the protein is cloned into an expression vector, for example into a binary vector, the expression vector is introduced into a host cell, for example into cells of the Arabidopsis thaliana wild type NASC N906 or any other plant cell as described above and/or below and/or in the examples, and the FCRP is expressed in said host cell.

In an embodiment of the present invention, the respective FCRP is localized after expression as indicated in column 6 of Table II, e.g. non-targeted, mitochondrial or plastidic.

In an embodiment of the present invention the FCRP is preferably produced in an compartment of the cell, e.g. in an organelle, for example in plastids or mitochondria. Ways of introducing nucleic acids into organelles, for example into plastids or mitochondria, and producing proteins in this compartment are known to the person skilled in the art.

The respective nucleic acid sequence according to the invention mentioned above is advantageously functionally joined to a nucleic acid sequence encoding a transit peptide, in such a manner that a preprotein is translated, which is able to direct the polypeptide to the organelle such as to plastids or mitochondria. In another preferred embodiment the nucleic acids according to the invention mentioned above is advantageously functionally joined to a promoter region functional in plastids or mitochondria like for example the RNA operon promoter fused to the 5′ UTR of the rbcL gene and in another preferred embodiment joined to a plastome sequences homologous to the integration sites. Example for useful integration sites are the trnV-rps12/7 (Skidar et al., Plant Cell Rep. 18, 20 (1998), and other reports), thr rbvL-aacD site (Svab et al., Proc. Natl. Acad. Sci. USA 90, 913 (1993)), the trnl-trnA site (De Cosa et al., Nat. Biotech. 19, 71 (2001)), the rps7-ndhB site (Hou et al., Transgenic Res. 12, 111 (2003)) and the ndhF-trnL site Zhang et al., Plant Physiol. 127, 131 (2001c)).

The nucleic acid sequence coding for the transit peptide is advantageously derived from a nucleic acid sequence encoding a protein finally resided in the plastids or mitochondria and is stemming from an organism selected from the group consisting of the genera Acetabularia, Arabidopsis, Brassica, Capsicum, Chlamydomonas, Cucurbita, Daucus, Dunaliella, Euglena, Flaveria, Glycine, Helianthus, Hordeum, Lactua, Lemna, Lolium, Lycopersion, Malus, Meticago, Mesembryanthemum, Nicotiana, Oenotherea, Oryza, Petúnia, Phaseolus, Physcomitrella, Pinus, Pisum, Pyrus, Raphanus, Saccharum, Silene, Sinapis, Solanum, Spinacea, Stevia, Synechococcus,Triticum and Zea.

Preferably the plastidal transit peptide is derived from the nucleic acid sequence encoding a protein selected from the group consisting of ribulose bisphosphate carboxylase/oxygenase, 5-enolpyruvyl-shikimate-3-phosphate synthase, acetolactate synthase, chloroplast ribosomal protein CS17, Cs protein, ferredoxin, plastocyanin, ribulose bisphosphate carboxylase activase, tryptophan synthase, acyl carrier protein, plastid chaperonin-60, cytochrome c552, 22-kDA heat shock protein, 33-kDa Oxygen-evolving enhancer protein 1, ATP synthase γ subunit, ATP synthase δ subunit, chlorophyll-a/b-binding proteinll-1, Oxygen-evolving enhancer protein 2, Oxygen-evolving enhancer protein 3, photosystem I: P21, photosystem I: P28, photosystem I: P30, photosystem I: P35, photosystem I: P37, glycerol-3-phosphate acyltransferases, chlorophyll a/b binding protein, CAB2 protein, hydroxymethyl-bilane synthase, pyruvate-orthophosphate dikinase, CAB3 protein, plastid ferritin, ferritin, early light-inducible protein, glutamate-1semialdehyde aminotransferase, protochlorophyllide reductase, starch-granule-bound amylase synthase, light-harvesting chlorophyll a/b-binding protein of photosystem II, major pollen allergen Lol p 5a, plastid CIpB ATP-dependent protease, superoxide dismutase, ferredoxin NADP oxidoreductase, 28-kDa ribonucleoprotein, 31-kDa ribonucleoprotein, 33-kDa ribonucleoprotein, acetolactate synthase, ATP synthase CFO subunit 1, ATP synthase CFO subunit 2, ATP synthase CFO subunit 3, ATP synthase CFO subunit 4, cytochrome f, ADP-glucose pyrophosphorylase, glutamine synthase, glutamine synthase 2, carbonic anhydrase, GapA protein, heatshock-protein hsp21, phosphate translocator, plastid CIpA ATP-dependent protease, plastid ribosomal protein CL24, plastid ribosomal protein CL9, plastid ribosomal protein PsCL18, plastid ribosomal protein PsCL25, DAHP synthase, starch phosphorylase, root acyl carrier protein II, betaine-aldehyde dehydrogenase, GapB protein, glutamine synthetase 2, phosphoribulokinase, nitrite reductase, ribosomal protein L12, ribosomal protein L13, ribosomal protein L21, ribosomal protein L35, ribosomal protein L40, triose phosphate-3-phosphoglyerate-phosphate translocator, ferredoxin-dependent glutamate synthase, glyceraldehyde-3-phosphate dehydrogenase, NADP-dependent malic enzyme and NADP-malate dehydrogenase. In an embodiment the plastome sequences are preferential derived from the plastome of the target organisms themselves and are advantegously derived from one of the following intergration sites: trnV-rps12/7 (Skidar et al., Plant Cell Rep. 18, 20 (1998) and other reports), rbvLaacD (Svab et al., Proc. Natl. Acad. Sci. USA 90, 913 (1993)), trnl-trnA (De Cosa et al., Nat. Biotech. 19, 71 (2001)), rps7-ndhB (Hou et al., Transgenic Res. 12, 111 (2003)) or ndhF-trnL site Zhang et al., Plant Physiol. 127, 131 (2001c)).

Advantageously the mitochondrial transit peptide is derived from the nucleic acid sequence encoding a protein selected from the group consisting of

22 kDA heat shock protein; 70 kDA heat shock protein; 83 kDA heat shock protein; 40S ribosomal protein S19; 50S ribosomal protein L15; ribosomal protein L29; 22 kDA PSST protein of complex I; 2-oxoacid dehydrogenase family protein; 2-oxoglutarate/malate translocator; 3-methyl-2-oxobutanoate hydroxymethyltransferase; 3-Methylcrotonyl-coenzyme A carboxylase (MCCase); 7,8-Dihydropteroate synthase (DHPS)/6-hydroxymethyl-7,8-dihydropterine pyrophosphokinase (HPPK); aconitate hydratase; acyl carrier protein (ACP); ADP/ATP translocase; alanyl-tRNA synthetase; alcohol dehydrogenase (ADH); alternative oxidase (AOX); aminoacyl-tRNA ligase; asparate aminotransferase; ATP synthase alpha subunit; ATP synthase beta subunit; ATP synthase delta subunit; ATP synthase epsilon subunit; ATP synthase gamma subunit; ATP-dependent Clp protease-proteolytic subunit; Chaperonin 60—- CPN60; Chaperonin 60 (2)—CPN60-2; Chaperonin 60(1)—CPN60-1; citrate synthase; cytochrome b-c1 complex subunit Rieske FeS Protein; cytochrome c reductase-processing peptidase subunit II; dihydrolipoamide S-acetyltransferase; farnesyl-diphosphate synthase 1; formate dehydrogenase; fumarate hydratase; gamma carbonic anhydrase protein (gammaCA); gamma carbonic anhydrase-like protein 1 (gammaCAL1); gamma carbonic anhydrase-like protein 2 (gammaCAL2); gamma-aminobutyric acid transaminase (GABA-T); glutathione reductase (GR); glycine decarboxylase subunit H; glycine decarboxylase subunit L; glycine decarboxylase subunit P; glycine decarboxylase subunit T; isovaleryl-CoA dehydrogenase (IVD); lipoamide dehydrogenase; malate oxidoreductase; manganese superoxide dismutase (Mn)SOD; methylmalonate-semialdehyde dehydrogenase; mitochondrial-processing peptidase beta subunit (MPP); mitochondrial-processing peptidase subunit alpha (MPP); monodehydroascorbate reductase (MDHAR); NAD dependent isocitrate dehydrogenase; NAD dependent malate dehydrogenase; NAD-dependent malic enzyme; NAD-dependent malic enzyme 59 kDa isoform; NAD-dependent malic enzyme 62 kDa isoform; NAD-dependent malic enzyme 65 kDa isoform; NADH ubiquinone oxidoreductase 29 kDa subunit; NADH-ubiquinone oxidoreductase 18 kDa subunit; NADH-ubiquinone oxidoreductase 20 kDa subunit; NADH-ubiquinone oxidoreductase 23 kDa subunit; NADH-ubiquinone oxidoreductase 75 kDa subunit; NADP dependent isocitrate dehydrogenase; NADP dependent malate dehydrogenase; nucleoside diphosphate kinase; nucleoside diphosphate kinase III; o-acetylserine (thiol) lyase (OAS-TL); propionyl-CoA carboxylase; protoporphyrinogen IX oxidase; pyruvate dehydrogenase E1 component subunit alpha; serine acetyltransferase (SAT); serine hydroxymethyltransferase; succinate dehydrogenase (SDH); succinic semialdehyde dehydrogenase (SSADH); succinyl-CoA ligase (GDP-forming) alpha-chain; succinyl-CoA ligase [GDP-forming] subunit beta; thiosulfate sulfurtransferase; threonyl-tRNA synthetase; trans-2-enoyl-CoA reductase; translocase inner membrane (TIM); translocase outer membrane (TOM); tRNA synthetase class I and ubiquinol cytochrome C oxidoreductase complex.

Further transit peptides and details thereto have been described above.

In another embodiment of the present invention the FCRP is produced without further targeting signal (e.g. as mentioned herein), e.g. in the cytoplasm of the cell. Ways of producing proteins without artificial targeting are known to the person skilled in the art.

The nucleic acid sequences used in the process are advantageously introduced in a nucleic acid construct, preferably an expression cassette, which makes possible the expression of the nucleic acid molecules in a non-human organism, advantageously a plant or a microorganism such as an algae, advantageously non-targeted or in an organelle, like plastids or mitochondria , respectively, of those non-human organisms.

Accordingly, the invention also relates to a nucleic acid construct, preferably to an expression construct, comprising the nucleic acid molecule of the present invention functionally linked to one or more regulatory elements or signals.

As described herein, the nucleic acid construct can also comprise further genes, which are to be introduced into the non-human organisms or cells. It is possible and advantageous to introduce into, and express in, the host organisms regulatory genes such as genes for inductors, repressors or enzymes, which, owing to their biological or enzymatic activity, engage in the regulation of one or more genes of the respective biosynthetic pathway. These genes can be of heterologous or homologous origin. Moreover, further biosynthesis genes may advantageously be present, or else these genes may be located on one or more further nucleic acid constructs. Genes, which are advantageously employed as further biosynthesis genes are genes of the fatty acid metabolism, amino acid metabolism, of glycolysis, of the tricarboxylic acid metabolism or their combinations. As described herein, regulator sequences or factors can have a positive effect on preferably the gene expression of the genes introduced, thus increasing it. Thus, an enhancement of the regulator elements may advantageously take place at the transcriptional level by using strong transcription signals such as promoters and/or enhancers. In addition, however, an enhancement of translation is also possible, for example by increasing mRNA stability or by inserting a translation enhancer sequence.

In principle, the nucleic acid construct can comprise the herein described regulator sequences and further sequences relevant for the expression of the comprised genes. Thus, the nucleic acid construct of the invention can be used as expression cassette and thus can be used directly for introduction into the plant, or else they may be introduced into a vector. Accordingly in one embodiment the nucleic acid construct is an expression cassette comprising a microorganism promoter or a microorganism terminator or both, beneath the nucleic acid molecule according to the invention. In another embodiment the expression cassette encompasses a plant promoter or a plant terminator or both, beneath the nucleic acid molecule according to the invention. In another embodiment the expression cassette encompasses sequences for transcription by organelle RNA polymerases, beneath the nucleic acid molecule according to the invention. In another embodiment an expression cassette encompasses a nucleic acid molecule encoding for a transit peptide, beneath the gene according to the invention.

In a preferred embodiment a nucleic acid construct, for example an expression cassette, comprises upstream, i.e. at the 5′ end of the encoding sequence, a promoter and downstream, i.e. at the 3′ end, a polyadenylation signal and optionally other regulatory elements which are operably linked to the intervening encoding sequence with one of the nucleic acids as depicted in the respective line in Table I, application no. 8, column 5 or 8, preferably the coding region thereof, or a homolog or a fragment thereof. By an operable linkage is meant the sequential arrangement of promoter, encoding sequence, terminator, optionally other regulatory elements and optionally targeting sequences in such a way that each of the regulatory elements/targeting sequence can fulfill its function in the expression of the encoding sequence in due manner. In one embodiment the sequences preferred for operable linkage are targeting sequences for ensuring subcellular localization in organelles, like plastids or mitochondria. However, targeting sequences for ensuring subcellular localization in the endoplasmic reticulum (=ER), in the nucleus, in oil corpuscles or other compartments may also be employed as well as translation promoters such as the 5′ lead sequence in tobacco mosaic virus (Gallie et al., Nucl. Acids Res. 15, 8693 (1987)).

As an example, a nucleic acid construct, like an expression cassette may, for example, contain a constitutive promoter or a tissue-specific promoter (preferably the USP or napin promoter), the gene to be expressed and a ER retention signal. For the ER retention signal the KDEL amino acid sequence (lysine, aspartic acid, glutamic acid, leucine) or the KKX amino acid sequence (lysine-lysine-X-stop, wherein X means every other known amino acid) is preferably employed.

In an embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 8, column 5 or 8, preferably shown in Table II A, application no. 8, column 5, or in Table II A, application no. 8, column 8, or in Table II B, application no. 8, column 8, or a homolog or a fragment thereof;
   (b) a nucleic acid molecule shown in Table I, application no. 8, column 5 or 8, preferably shown in Table I A, application no. 8, column 5, or in Table I A, application no. 8, column 8, or in Table I B, application no. 8, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 8, preferably shown in Table II A, application no. 8, column 5, or in Table II A, application no. 8, column 8, or in Table II B, application no. 8, column 8;
   (d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 8, preferably shown in Table I A, application no. 8, column 5, or in Table I A, application no. 8, column 8, or in Table I B, application no. 8, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 8;
   (i) a nucleic acid molecule encoding a polypeptide having the activity represented by a respective protein as depicted in column 5 of Table II, application no. 8;
   (j) a nucleic acid molecule comprising a polynucleotide which is obtained by amplifying a cDNA library or a genomic library using the primers in Table III, application no. 8, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto.
      and, optionally
3) nucleic acid molecule encoding a transit peptide, preferably encoding a plastidal transit peptide or a mitochondrial transit peptide;
   which are operable linked.
   (All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 8.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 8 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 8, in the same line as of the respective nucleic acid molecule according to 2) “plastidic” is depicted, or encoding a mitochondrial transit peptide, especially in case in column 6 of Table I, application no. 8, in the same line as of the respective nucleic acid molecule according to 2) “mitochondrial” is depicted.

In another embodiment thereof the expression cassette does not comprise 3) a nucleic acid molecule encoding a transit peptide, especially in case in column 6 of Table I, application no. 8, in the same line as of the respective nucleic acid molecule according to 2) “non-targeted” is depicted.

In a further embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 8, column 5 or 8, preferably shown in Table II A, application no. 8, column 5, or in Table II A, application no. 8, column 8, or in Table II B, application no. 8, column 8, or a homolog or a fragment thereof;
   (b) a nucleic acid molecule shown in Table I, application no. 8, column 5 or 8, preferably shown in Table I A, application no. 8, column 5, or in Table I A, application no. 8, column 8, or in Table I B, application no. 8, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 8, preferably shown in Table II A, application no. 8, column 5, or in Table II A, application no. 8, column 8, or in Table II B, application no. 8, column 8;
   (d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 8, preferably shown in Table I A, application no. 8, column 5, or in Table I A, application no. 8, column 8, or in Table I B, application no. 8, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 8;
   (i) a nucleic acid molecule encoding a polypeptide having the activity represented by a respective protein as depicted in column 5 of Table II, application no. 8;
   (j) a nucleic acid molecule comprising a polynucleotide which is obtained by amplifying a cDNA library or a genomic library using the primers in Table III, application no. 8, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, optionally
3) nucleic acid molecule encoding a transit peptide, preferably encoding a plastidal transit peptide or a mitochondrial transit peptide;
   which are operable linked and
   whereby, preferably, the nucleic acid molecule according to 2 (a), (b), (c), (d), (e), (f), (g), (h), (i), (j) or (k) distinguishes over the sequence depicted in Table I A and/or I B, application no. 8, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention according to 2) does not consist of the sequence shown in Table I A and/or I B, application no. 8, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 8, column 5 or 8, or the coding region thereof, respectively. In a further embodiment the nucleic acid molecule does not encode the polypeptide sequence shown in Table II A and/or II B, application no. 8, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 8, column 5 or 8 and does not encode a protein of the sequence shown in Table II A and/or II B, application no. 8, column 5 or 8. Accordingly, in one embodiment, the protein encoded by a sequence of a nucleic acid according to 2 (a), (b), (c), (d), (e), (f), (g), (h), (i), (j) or (k) does not consist of the sequence shown in Table I A and/or I B, application no. 8, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 8, column 5 or 8 but less than 100%, 99.999%, 99.99%, 99.9%, 99%, 98%, 97%, 96% or 95% identical to the sequence shown in Table II A and/or II B, application no. 8, column 5 or 8.
   (All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 8.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 8 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 8, in the same line as of the respective nu oleic acid molecule according to 2) “plastidic” is depicted, or encoding a mitochondrial transit peptide, especially in case in column 6 of Table I, application no. 8, in the same line as of the respective nucleic acid molecule according to 2) “mitochondrial” is depicted.

In another embodiment thereof the expression cassette does not comprise 3) a nucleic acid molecule encoding a transit peptide, especially in case in column 6 of Table I, application no. 8, in the same line as of the respective nucleic acid molecule according to 2) “non-targeted” is depicted.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 113651, or a homolog or fragment thereof, or a homolog thereof being depicted in Table I, application no. 8, column 8, in the same line as SEQ ID NO. 113651, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 113651 is depicted, plastidic is mentioned, or a mitochondrial transit peptide in case in Table I, application no. 8, in column 6 in the same line as SEQ ID NO. 113651 is depicted, mitochondrial is mentioned; which is operable linked, too.

In another embodiment thereof the expression cassette does not comprise an additional transit peptide in case in Table I, application no. 8, in column 6 in the same line as SEQ ID NO. 113651 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17637, or a homolog or fragment thereof, or a homolog thereof being depicted in Table I, application no. 8, column 8, in the same line as SEQ ID NO. 17637, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 17637 is depicted, plastidic is mentioned, or a mitochondrial transit peptide in case in Table I, application no. 8, in column 6 in the same line as SEQ ID NO. 17637 is depicted, mitochondrial is mentioned; which is operable linked, too.

In another embodiment thereof the expression cassette does not comprise an additional transit peptide in case in Table I, application no. 8, in column 6 in the same line as SEQ ID NO. 17637 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 1061, or a homolog or fragment thereof, or a homolog thereof being depicted in Table I, application no. 8, column 8, in the same line as SEQ ID NO. 1061, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 1061 is depicted, plastidic is mentioned, or a mitochondrial transit peptide in case in Table I, application no. 8, in column 6 in the same line as SEQ ID NO. 1061 is depicted, mitochondrial is mentioned; which is operable linked, too.

In another embodiment thereof the expression cassette does not comprise an additional transit peptide in case in Table I, application no. 8, in column 6 in the same line as SEQ ID NO. 1061 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 113714, or a homolog or fragment thereof, or a homolog thereof being depicted in Table I, application no. 8, column 8, in the same line as SEQ ID NO. 113714, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 113714 is depicted, plastidic is mentioned, or a mitochondrial transit peptide in case in Table I, application no. 8, in column 6 in the same line as SEQ ID NO. 113714 is depicted, mitochondrial is mentioned; which is operable linked, too.

In another embodiment thereof the expression cassette does not comprise an additional transit peptide in case in Table I, application no. 8, in column 6 in the same line as SEQ ID NO. 113714 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 1623, or a homolog or fragment thereof, or a homolog thereof being depicted in Table I, application no. 8, column 8, in the same line as SEQ ID NO. 1623, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 1623 is depicted, plastidic is mentioned, or a mitochondrial transit peptide in case in Table I, application no. 8, in column 6 in the same line as SEQ ID NO. 1623 is depicted, mitochondrial is mentioned; which is operable linked, too.

In another embodiment thereof the expression cassette does not comprise an additional transit peptide in case in Table I, application no. 8, in column 6 in the same line as SEQ ID NO. 1623 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68657, or a homolog or fragment thereof, or a homolog thereof being depicted in Table I, application no. 8, column 8, in the same line as SEQ ID NO. 68657, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 68657 is depicted, plastidic is mentioned, or a mitochondrial transit peptide in case in Table I, application no. 8, in column 6 in the same line as SEQ ID NO. 68657 is depicted, mitochondrial is mentioned; which is operable linked, too.

In another embodiment thereof the expression cassette does not comprise an additional transit peptide in case in Table I, application no. 8, in column 6 in the same line as SEQ ID NO. 68657 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 22921, or a homolog or fragment thereof, or a homolog thereof being depicted in Table I, application no. 8, column 8, in the same line as SEQ ID NO. 22921, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 22921 is depicted, plastidic is mentioned, or a mitochondrial transit peptide in case in Table I, application no. 8, in column 6 in the same line as SEQ ID NO. 22921 is depicted, mitochondrial is mentioned; which is operable linked, too.

In another embodiment thereof the expression cassette does not comprise an additional transit peptide in case in Table I, application no. 8, in column 6 in the same line as SEQ ID NO. 22921 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 2935, or a homolog or fragment thereof, or a homolog thereof being depicted in Table I, application no. 8, column 8, in the same line as SEQ ID NO. 2935, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 2935 is depicted, plastidic is mentioned, or a mitochondrial transit peptide in case in Table I, application no. 8, in column 6 in the same line as SEQ ID NO. 2935 is depicted, mitochondrial is mentioned; which is operable linked, too.

In another embodiment thereof the expression cassette does not comprise an additional transit peptide in case in Table I, application no. 8, in column 6 in the same line as SEQ ID NO. 2935 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 109717, or a homolog or fragment thereof, or a homolog thereof being depicted in Table I, application no. 8, column 8, in the same line as SEQ ID NO. 109717, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 109717 is depicted, plastidic is mentioned, or a mitochondrial transit peptide in case in Table I, application no. 8, in column 6 in the same line as SEQ ID NO. 109717 is depicted, mitochondrial is mentioned; which is operable linked, too.

In another embodiment thereof the expression cassette does not comprise an additional transit peptide in case in Table I, application no. 8, in column 6 in the same line as SEQ ID NO. 109717 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 23482, or a homolog or fragment thereof, or a homolog thereof being depicted in Table I, application no. 8, column 8, in the same line as SEQ ID NO. 23482, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 23482 is depicted, plastidic is mentioned, or a mitochondrial transit peptide in case in Table I, application no. 8, in column 6 in the same line as SEQ ID NO. 23482 is depicted, mitochondrial is mentioned; which is operable linked, too.

In another embodiment thereof the expression cassette does not comprise an additional transit peptide in case in Table I, application no. 8, in column 6 in the same line as SEQ ID NO. 23482 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 3279, or a homolog or fragment thereof, or a homolog thereof being depicted in Table I, application no. 8, column 8, in the same line as SEQ ID NO. 3279, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 3279 is depicted, plastidic is mentioned, or a mitochondrial transit peptide in case in Table I, application no. 8, in column 6 in the same line as SEQ ID NO. 3279 is depicted, mitochondrial is mentioned; which is operable linked, too.

In another embodiment thereof the expression cassette does not comprise an additional transit peptide in case in Table I, application no. 8, in column 6 in the same line as SEQ ID NO. 3279 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68849, or a homolog or fragment thereof, or a homolog thereof being depicted in Table I, application no. 8, column 8, in the same line as SEQ ID NO. 68849, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 68849 is depicted, plastidic is mentioned, or a mitochondrial transit peptide in case in Table I, application no. 8, in column 6 in the same line as SEQ ID NO. 68849 is depicted, mitochondrial is mentioned; which is operable linked, too.

In another embodiment thereof the expression cassette does not comprise an additional transit peptide in case in Table I, application no. 8, in column 6 in the same line as SEQ ID NO. 68849 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4102, or a homolog or fragment thereof, or a homolog thereof being depicted in Table I, application no. 8, column 8, in the same line as SEQ ID NO. 4102, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 4102 is depicted, plastidic is mentioned, or a mitochondrial transit peptide in case in Table I, application no. 8, in column 6 in the same line as SEQ ID NO. 4102 is depicted, mitochondrial is mentioned; which is operable linked, too.

In another embodiment thereof the expression cassette does not comprise an additional transit peptide in case in Table I, application no. 8, in column 6 in the same line as SEQ ID NO. 4102 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 24438, or a homolog or fragment thereof, or a homolog thereof being depicted in Table I, application no. 8, column 8, in the same line as SEQ ID NO. 24438, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 24438 is depicted, plastidic is mentioned, or a mitochondrial transit peptide in case in Table I, application no. 8, in column 6 in the same line as SEQ ID NO. 24438 is depicted, mitochondrial is mentioned; which is operable linked, too.

In another embodiment thereof the expression cassette does not comprise an additional transit peptide in case in Table I, application no. 8, in column 6 in the same line as SEQ ID NO. 24438 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 25498, or a homolog or fragment thereof, or a homolog thereof being depicted in Table I, application no. 8, column 8, in the same line as SEQ ID NO. 25498, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 25498 is depicted, plastidic is mentioned, or a mitochondrial transit peptide in case in Table I, application no. 8, in column 6 in the same line as SEQ ID NO. 25498 is depicted, mitochondrial is mentioned; which is operable linked, too.

In another embodiment thereof the expression cassette does not comprise an additional transit peptide in case in Table I, application no. 8, in column 6 in the same line as SEQ ID NO. 25498 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 114083, or a homolog or fragment thereof, or a homolog thereof being depicted in Table I, application no. 8, column 8, in the same line as SEQ ID NO. 114083, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 114083 is depicted, plastidic is mentioned, or a mitochondrial transit peptide in case in Table I, application no. 8, in column 6 in the same line as SEQ ID NO. 114083 is depicted, mitochondrial is mentioned; which is operable linked, too.

In another embodiment thereof the expression cassette does not comprise an additional transit peptide in case in Table I, application no. 8, in column 6 in the same line as SEQ ID NO. 114083 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 114198, or a homolog or fragment thereof, or a homolog thereof being depicted in Table I, application no. 8, column 8, in the same line as SEQ ID NO. 114198, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 114198 is depicted, plastidic is mentioned, or a mitochondrial transit peptide in case in Table I, application no. 8, in column 6 in the same line as SEQ ID NO. 114198 is depicted, mitochondrial is mentioned; which is operable linked, too.

In another embodiment thereof the expression cassette does not comprise an additional transit peptide in case in Table I, application no. 8, in column 6 in the same line as SEQ ID NO. 114198 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6040, or a homolog or fragment thereof, or a homolog thereof being depicted in Table I, application no. 8, column 8, in the same line as SEQ ID NO. 6040, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 6040 is depicted, plastidic is mentioned, or a mitochondrial transit peptide in case in Table I, application no. 8, in column 6 in the same line as SEQ ID NO. 6040 is depicted, mitochondrial is mentioned; which is operable linked, too.

In another embodiment thereof the expression cassette does not comprise an additional transit peptide in case in Table I, application no. 8, in column 6 in the same line as SEQ ID NO. 6040 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 29500, or a homolog or fragment thereof, or a homolog thereof being depicted in Table I, application no. 8, column 8, in the same line as SEQ ID NO. 29500, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 29500 is depicted, plastidic is mentioned, or a mitochondrial transit peptide in case in Table I, application no. 8, in column 6 in the same line as SEQ ID NO. 29500 is depicted, mitochondrial is mentioned; which is operable linked, too.

In another embodiment thereof the expression cassette does not comprise an additional transit peptide in case in Table I, application no. 8, in column 6 in the same line as SEQ ID NO. 29500 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 111155, or a homolog or fragment thereof, or a homolog thereof being depicted in Table I, application no. 8, column 8, in the same line as SEQ ID NO. 111155, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 111155 is depicted, plastidic is mentioned, or a mitochondrial transit peptide in case in Table I, application no. 8, in column 6 in the same line as SEQ ID NO. 111155 is depicted, mitochondrial is mentioned; which is operable linked, too.

In another embodiment thereof the expression cassette does not comprise an additional transit peptide in case in Table I, application no. 8, in column 6 in the same line as SEQ ID NO. 111155 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 114231, or a homolog or fragment thereof, or a homolog thereof being depicted in Table I, application no. 8, column 8, in the same line as SEQ ID NO. 114231, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 114231 is depicted, plastidic is mentioned, or a mitochondrial transit peptide in case in Table I, application no. 8, in column 6 in the same line as SEQ ID NO. 114231 is depicted, mitochondrial is mentioned; which is operable linked, too.

In another embodiment thereof the expression cassette does not comprise an additional transit peptide in case in Table I, application no. 8, in column 6 in the same line as SEQ ID NO. 114231 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6510, or a homolog or fragment thereof, or a homolog thereof being depicted in Table I, application no. 8, column 8, in the same line as SEQ ID NO. 6510, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 6510 is depicted, plastidic is mentioned, or a mitochondrial transit peptide in case in Table I, application no. 8, in column 6 in the same line as SEQ ID NO. 6510 is depicted, mitochondrial is mentioned; which is operable linked, too.

In another embodiment thereof the expression cassette does not comprise an additional transit peptide in case in Table I, application no. 8, in column 6 in the same line as SEQ ID NO. 6510 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7686, or a homolog or fragment thereof, or a homolog thereof being depicted in Table I, application no. 8, column 8, in the same line as SEQ ID NO. 7686, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 7686 is depicted, plastidic is mentioned, or a mitochondrial transit peptide in case in Table I, application no. 8, in column 6 in the same line as SEQ ID NO. 7686 is depicted, mitochondrial is mentioned; which is operable linked, too.

In another embodiment thereof the expression cassette does not comprise an additional transit peptide in case in Table I, application no. 8, in column 6 in the same line as SEQ ID NO. 7686 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 75807, or a homolog or fragment thereof, or a homolog thereof being depicted in Table I, application no. 8, column 8, in the same line as SEQ ID NO. 75807, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 75807 is depicted, plastidic is mentioned, or a mitochondrial transit peptide in case in Table I, application no. 8, in column 6 in the same line as SEQ ID NO. 75807 is depicted, mitochondrial is mentioned; which is operable linked, too.

In another embodiment thereof the expression cassette does not comprise an additional transit peptide in case in Table I, application no. 8, in column 6 in the same line as SEQ ID NO. 75807 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 114552, or a homolog or fragment thereof, or a homolog thereof being depicted in Table I, application no. 8, column 8, in the same line as SEQ ID NO. 114552, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 114552 is depicted, plastidic is mentioned, or a mitochondrial transit peptide in case in Table I, application no. 8, in column 6 in the same line as SEQ ID NO. 114552 is depicted, mitochondrial is mentioned; which is operable linked, too.

In another embodiment thereof the expression cassette does not comprise an additional transit peptide in case in Table I, application no. 8, in column 6 in the same line as SEQ ID NO. 114552 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 114979, or a homolog or fragment thereof, or a homolog thereof being depicted in Table I, application no. 8, column 8, in the same line as SEQ ID NO. 114979, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 114979 is depicted, plastidic is mentioned, or a mitochondrial transit peptide in case in Table I, application no. 8, in column 6 in the same line as SEQ ID NO. 114979 is depicted, mitochondrial is mentioned; which is operable linked, too.

In another embodiment thereof the expression cassette does not comprise an additional transit peptide in case in Table I, application no. 8, in column 6 in the same line as SEQ ID NO. 114979 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38226, or a homolog or fragment thereof, or a homolog thereof being depicted in Table I, application no. 8, column 8, in the same line as SEQ ID NO. 38226, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 38226 is depicted, plastidic is mentioned, or a mitochondrial transit peptide in case in Table I, application no. 8, in column 6 in the same line as SEQ ID NO. 38226 is depicted, mitochondrial is mentioned; which is operable linked, too.

In another embodiment thereof the expression cassette does not comprise an additional transit peptide in case in Table I, application no. 8, in column 6 in the same line as SEQ ID NO. 38226 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38266, or a homolog or fragment thereof, or a homolog thereof being depicted in Table I, application no. 8, column 8, in the same line as SEQ ID NO. 38266, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 38266 is depicted, plastidic is mentioned, or a mitochondrial transit peptide in case in Table I, application no. 8, in column 6 in the same line as SEQ ID NO. 38266 is depicted, mitochondrial is mentioned; which is operable linked, too.

In another embodiment thereof the expression cassette does not comprise an additional transit peptide in case in Table I, application no. 8, in column 6 in the same line as SEQ ID NO. 38266 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115056, or a homolog or fragment thereof, or a homolog thereof being depicted in Table I, application no. 8, column 8, in the same line as SEQ ID NO. 115056, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 115056 is depicted, plastidic is mentioned, or a mitochondrial transit peptide in case in Table I, application no. 8, in column 6 in the same line as SEQ ID NO. 115056 is depicted, mitochondrial is mentioned; which is operable linked, too.

In another embodiment thereof the expression cassette does not comprise an additional transit peptide in case in Table I, application no. 8, in column 6 in the same line as SEQ ID NO. 115056 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78154, or a homolog or fragment thereof, or a homolog thereof being depicted in Table I, application no. 8, column 8, in the same line as SEQ ID NO. 78154, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 78154 is depicted, plastidic is mentioned, or a mitochondrial transit peptide in case in Table I, application no. 8, in column 6 in the same line as SEQ ID NO. 78154 is depicted, mitochondrial is mentioned; which is operable linked, too.

In another embodiment thereof the expression cassette does not comprise an additional transit peptide in case in Table I, application no. 8, in column 6 in the same line as SEQ ID NO. 78154 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38300, or a homolog or fragment thereof, or a homolog thereof being depicted in Table I, application no. 8, column 8, in the same line as SEQ ID NO. 38300, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 38300 is depicted, plastidic is mentioned, or a mitochondrial transit peptide in case in Table I, application no. 8, in column 6 in the same line as SEQ ID NO. 38300 is depicted, mitochondrial is mentioned; which is operable linked, too.

In another embodiment thereof the expression cassette does not comprise an additional transit peptide in case in Table I, application no. 8, in column 6 in the same line as SEQ ID NO. 38300 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115064, or a homolog or fragment thereof, or a homolog thereof being depicted in Table I, application no. 8, column 8, in the same line as SEQ ID NO. 115064, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 115064 is depicted, plastidic is mentioned, or a mitochondrial transit peptide in case in Table I, application no. 8, in column 6 in the same line as SEQ ID NO. 115064 is depicted, mitochondrial is mentioned; which is operable linked, too.

In another embodiment thereof the expression cassette does not comprise an additional transit peptide in case in Table I, application no. 8, in column 6 in the same line as SEQ ID NO. 115064 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38345, or a homolog or fragment thereof, or a homolog thereof being depicted in Table I, application no. 8, column 8, in the same line as SEQ ID NO. 38345, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 38345 is depicted, plastidic is mentioned, or a mitochondrial transit peptide in case in Table I, application no. 8, in column 6 in the same line as SEQ ID NO. 38345 is depicted, mitochondrial is mentioned; which is operable linked, too.

In another embodiment thereof the expression cassette does not comprise an additional transit peptide in case in Table I, application no. 8, in column 6 in the same line as SEQ ID NO. 38345 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78753, or a homolog or fragment thereof, or a homolog thereof being depicted in Table I, application no. 8, column 8, in the same line as SEQ ID NO. 78753, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 78753 is depicted, plastidic is mentioned, or a mitochondrial transit peptide in case in Table I, application no. 8, in column 6 in the same line as SEQ ID NO. 78753 is depicted, mitochondrial is mentioned; which is operable linked, too.

In another embodiment thereof the expression cassette does not comprise an additional transit peptide in case in Table I, application no. 8, in column 6 in the same line as SEQ ID NO. 78753 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78953, or a homolog or fragment thereof, or a homolog thereof being depicted in Table I, application no. 8, column 8, in the same line as SEQ ID NO. 78953, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 78953 is depicted, plastidic is mentioned, or a mitochondrial transit peptide in case in Table I, application no. 8, in column 6 in the same line as SEQ ID NO. 78953 is depicted, mitochondrial is mentioned; which is operable linked, too.

In another embodiment thereof the expression cassette does not comprise an additional transit peptide in case in Table I, application no. 8, in column 6 in the same line as SEQ ID NO. 78953 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 8920, or a homolog or fragment thereof, or a homolog thereof being depicted in Table I, application no. 8, column 8, in the same line as SEQ ID NO. 8920, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 8920 is depicted, plastidic is mentioned, or a mitochondrial transit peptide in case in Table I, application no. 8, in column 6 in the same line as SEQ ID NO. 8920 is depicted, mitochondrial is mentioned; which is operable linked, too.

In another embodiment thereof the expression cassette does not comprise an additional transit peptide in case in Table I, application no. 8, in column 6 in the same line as SEQ ID NO. 8920 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 112193, or a homolog or fragment thereof, or a homolog thereof being depicted in Table I, application no. 8, column 8, in the same line as SEQ ID NO. 112193, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 112193 is depicted, plastidic is mentioned, or a mitochondrial transit peptide in case in Table I, application no. 8, in column 6 in the same line as SEQ ID NO. 112193 is depicted, mitochondrial is mentioned; which is operable linked, too.

In another embodiment thereof the expression cassette does not comprise an additional transit peptide in case in Table I, application no. 8, in column 6 in the same line as SEQ ID NO. 112193 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115595, or a homolog or fragment thereof, or a homolog thereof being depicted in Table I, application no. 8, column 8, in the same line as SEQ ID NO. 115595, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 115595 is depicted, plastidic is mentioned, or a mitochondrial transit peptide in case in Table I, application no. 8, in column 6 in the same line as SEQ ID NO. 115595 is depicted, mitochondrial is mentioned; which is operable linked, too.

In another embodiment thereof the expression cassette does not comprise an additional transit peptide in case in Table I, application no. 8, in column 6 in the same line as SEQ ID NO. 115595 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39002, or a homolog or fragment thereof, or a homolog thereof being depicted in Table I, application no. 8, column 8, in the same line as SEQ ID NO. 39002, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 39002 is depicted, plastidic is mentioned, or a mitochondrial transit peptide in case in Table I, application no. 8, in column 6 in the same line as SEQ ID NO. 39002 is depicted, mitochondrial is mentioned; which is operable linked, too.

In another embodiment thereof the expression cassette does not comprise an additional transit peptide in case in Table I, application no. 8, in column 6 in the same line as SEQ ID NO. 39002 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39040, or a homolog or fragment thereof, or a homolog thereof being depicted in Table I, application no. 8, column 8, in the same line as SEQ ID NO. 39040, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 39040 is depicted, plastidic is mentioned, or a mitochondrial transit peptide in case in Table I, application no. 8, in column 6 in the same line as SEQ ID NO. 39040 is depicted, mitochondrial is mentioned; which is operable linked, too.

In another embodiment thereof the expression cassette does not comprise an additional transit peptide in case in Table I, application no. 8, in column 6 in the same line as SEQ ID NO. 39040 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39237, or a homolog or fragment thereof, or a homolog thereof being depicted in Table I, application no. 8, column 8, in the same line as SEQ ID NO. 39237, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 39237 is depicted, plastidic is mentioned, or a mitochondrial transit peptide in case in Table I, application no. 8, in column 6 in the same line as SEQ ID NO. 39237 is depicted, mitochondrial is mentioned; which is operable linked, too.

In another embodiment thereof the expression cassette does not comprise an additional transit peptide in case in Table I, application no. 8, in column 6 in the same line as SEQ ID NO. 39237 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115628, or a homolog or fragment thereof, or a homolog thereof being depicted in Table I, application no. 8, column 8, in the same line as SEQ ID NO. 115628, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 115628 is depicted, plastidic is mentioned, or a mitochondrial transit peptide in case in Table I, application no. 8, in column 6 in the same line as SEQ ID NO. 115628 is depicted, mitochondrial is mentioned; which is operable linked, too.

In another embodiment thereof the expression cassette does not comprise an additional transit peptide in case in Table I, application no. 8, in column 6 in the same line as SEQ ID NO. 115628 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40329, or a homolog or fragment thereof, or a homolog thereof being depicted in Table I, application no. 8, column 8, in the same line as SEQ ID NO. 40329, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 40329 is depicted, plastidic is mentioned, or a mitochondrial transit peptide in case in Table I, application no. 8, in column 6 in the same line as SEQ ID NO. 40329 is depicted, mitochondrial is mentioned; which is operable linked, too.

In another embodiment thereof the expression cassette does not comprise an additional transit peptide in case in Table I, application no. 8, in column 6 in the same line as SEQ ID NO. 40329 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40665, or a homolog or fragment thereof, or a homolog thereof being depicted in Table I, application no. 8, column 8, in the same line as SEQ ID NO. 40665, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 40665 is depicted, plastidic is mentioned, or a mitochondrial transit peptide in case in Table I, application no. 8, in column 6 in the same line as SEQ ID NO. 40665 is depicted, mitochondrial is mentioned; which is operable linked, too.

In another embodiment thereof the expression cassette does not comprise an additional transit peptide in case in Table I, application no. 8, in column 6 in the same line as SEQ ID NO. 40665 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115655, or a homolog or fragment thereof, or a homolog thereof being depicted in Table I, application no. 8, column 8, in the same line as SEQ ID NO. 115655, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 115655 is depicted, plastidic is mentioned, or a mitochondrial transit peptide in case in Table I, application no. 8, in column 6 in the same line as SEQ ID NO. 115655 is depicted, mitochondrial is mentioned; which is operable linked, too.

In another embodiment thereof the expression cassette does not comprise an additional transit peptide in case in Table I, application no. 8, in column 6 in the same line as SEQ ID NO. 115655 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115719, or a homolog or fragment thereof, or a homolog thereof being depicted in Table I, application no. 8, column 8, in the same line as SEQ ID NO. 115719, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 115719 is depicted, plastidic is mentioned, or a mitochondrial transit peptide in case in Table I, application no. 8, in column 6 in the same line as SEQ ID NO. 115719 is depicted, mitochondrial is mentioned; which is operable linked, too.

In another embodiment thereof the expression cassette does not comprise an additional transit peptide in case in Table I, application no. 8, in column 6 in the same line as SEQ ID NO. 115719 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115791, or a homolog or fragment thereof, or a homolog thereof being depicted in Table I, application no. 8, column 8, in the same line as SEQ ID NO. 115791, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 115791 is depicted, plastidic is mentioned, or a mitochondrial transit peptide in case in Table I, application no. 8, in column 6 in the same line as SEQ ID NO. 115791 is depicted, mitochondrial is mentioned; which is operable linked, too.

In another embodiment thereof the expression cassette does not comprise an additional transit peptide in case in Table I, application no. 8, in column 6 in the same line as SEQ ID NO. 115791 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 81980, or a homolog or fragment thereof, or a homolog thereof being depicted in Table I, application no. 8, column 8, in the same line as SEQ ID NO. 81980, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 81980 is depicted, plastidic is mentioned, or a mitochondrial transit peptide in case in Table I, application no. 8, in column 6 in the same line as SEQ ID NO. 81980 is depicted, mitochondrial is mentioned; which is operable linked, too.

In another embodiment thereof the expression cassette does not comprise an additional transit peptide in case in Table I, application no. 8, in column 6 in the same line as SEQ ID NO. 81980 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115864, or a homolog or fragment thereof, or a homolog thereof being depicted in Table I, application no. 8, column 8, in the same line as SEQ ID NO. 115864, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 115864 is depicted, plastidic is mentioned, or a mitochondrial transit peptide in case in Table I, application no. 8, in column 6 in the same line as SEQ ID NO. 115864 is depicted, mitochondrial is mentioned; which is operable linked, too.

In another embodiment thereof the expression cassette does not comprise an additional transit peptide in case in Table I, application no. 8, in column 6 in the same line as SEQ ID NO. 115864 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 41006, or a homolog or fragment thereof, or a homolog thereof being depicted in Table I, application no. 8, column 8, in the same line as SEQ ID NO. 41006, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 41006 is depicted, plastidic is mentioned, or a mitochondrial transit peptide in case in Table I, application no. 8, in column 6 in the same line as SEQ ID NO. 41006 is depicted, mitochondrial is mentioned; which is operable linked, too.

In another embodiment thereof the expression cassette does not comprise an additional transit peptide in case in Table I, application no. 8, in column 6 in the same line as SEQ ID NO. 41006 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 116110, or a homolog or fragment thereof, or a homolog thereof being depicted in Table I, application no. 8, column 8, in the same line as SEQ ID NO. 116110, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 116110 is depicted, plastidic is mentioned, or a mitochondrial transit peptide in case in Table I, application no. 8, in column 6 in the same line as SEQ ID NO. 116110 is depicted, mitochondrial is mentioned; which is operable linked, too.

In another embodiment thereof the expression cassette does not comprise an additional transit peptide in case in Table I, application no. 8, in column 6 in the same line as SEQ ID NO. 116110 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 116172, or a homolog or fragment thereof, or a homolog thereof being depicted in Table I, application no. 8, column 8, in the same line as SEQ ID NO. 116172, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 116172 is depicted, plastidic is mentioned, or a mitochondrial transit peptide in case in Table I, application no. 8, in column 6 in the same line as SEQ ID NO. 116172 is depicted, mitochondrial is mentioned; which is operable linked, too.

In another embodiment thereof the expression cassette does not comprise an additional transit peptide in case in Table I, application no. 8, in column 6 in the same line as SEQ ID NO. 116172 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 10726, or a homolog or fragment thereof, or a homolog thereof being depicted in Table I, application no. 8, column 8, in the same line as SEQ ID NO. 10726, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 10726 is depicted, plastidic is mentioned, or a mitochondrial transit peptide in case in Table I, application no. 8, in column 6 in the same line as SEQ ID NO. 10726 is depicted, mitochondrial is mentioned; which is operable linked, too.

In another embodiment thereof the expression cassette does not comprise an additional transit peptide in case in Table I, application no. 8, in column 6 in the same line as SEQ ID NO. 10726 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 100675, or a homolog or fragment thereof, or a homolog thereof being depicted in Table I, application no. 8, column 8, in the same line as SEQ ID NO. 100675, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 100675 is depicted, plastidic is mentioned, or a mitochondrial transit peptide in case in Table I, application no. 8, in column 6 in the same line as SEQ ID NO. 100675 is depicted, mitochondrial is mentioned; which is operable linked, too.

In another embodiment thereof the expression cassette does not comprise an additional transit peptide in case in Table I, application no. 8, in column 6 in the same line as SEQ ID NO. 100675 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 116312, or a homolog or fragment thereof, or a homolog thereof being depicted in Table I, application no. 8, column 8, in the same line as SEQ ID NO. 116312, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 116312 is depicted, plastidic is mentioned, or a mitochondrial transit peptide in case in Table I, application no. 8, in column 6 in the same line as SEQ ID NO. 116312 is depicted, mitochondrial is mentioned; which is operable linked, too.

In another embodiment thereof the expression cassette does not comprise an additional transit peptide in case in Table I, application no. 8, in column 6 in the same line as SEQ ID NO. 116312 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 85723, or a homolog or fragment thereof, or a homolog thereof being depicted in Table I, application no. 8, column 8, in the same line as SEQ ID NO. 85723, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 85723 is depicted, plastidic is mentioned, or a mitochondrial transit peptide in case in Table I, application no. 8, in column 6 in the same line as SEQ ID NO. 85723 is depicted, mitochondrial is mentioned; which is operable linked, too.

In another embodiment thereof the expression cassette does not comprise an additional transit peptide in case in Table I, application no. 8, in column 6 in the same line as SEQ ID NO. 85723 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 52660, or a homolog or fragment thereof, or a homolog thereof being depicted in Table I, application no. 8, column 8, in the same line as SEQ ID NO. 52660, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 52660 is depicted, plastidic is mentioned, or a mitochondrial transit peptide in case in Table I, application no. 8, in column 6 in the same line as SEQ ID NO. 52660 is depicted, mitochondrial is mentioned; which is operable linked, too.

In another embodiment thereof the expression cassette does not comprise an additional transit peptide in case in Table I, application no. 8, in column 6 in the same line as SEQ ID NO. 52660 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 53878, or a homolog or fragment thereof, or a homolog thereof being depicted in Table I, application no. 8, column 8, in the same line as SEQ ID NO. 53878, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 53878 is depicted, plastidic is mentioned, or a mitochondrial transit peptide in case in Table I, application no. 8, in column 6 in the same line as SEQ ID NO. 53878 is depicted, mitochondrial is mentioned; which is operable linked, too.

In another embodiment thereof the expression cassette does not comprise an additional transit peptide in case in Table I, application no. 8, in column 6 in the same line as SEQ ID NO. 53878 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 95047, or a homolog or fragment thereof, or a homolog thereof being depicted in Table I, application no. 8, column 8, in the same line as SEQ ID NO. 95047, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 95047 is depicted, plastidic is mentioned, or a mitochondrial transit peptide in case in Table I, application no. 8, in column 6 in the same line as SEQ ID NO. 95047 is depicted, mitochondrial is mentioned; which is operable linked, too.

In another embodiment thereof the expression cassette does not comprise an additional transit peptide in case in Table I, application no. 8, in column 6 in the same line as SEQ ID NO. 95047 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 11471, or a homolog or fragment thereof, or a homolog thereof being depicted in Table I, application no. 8, column 8, in the same line as SEQ ID NO. 11471, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 11471 is depicted, plastidic is mentioned, or a mitochondrial transit peptide in case in Table I, application no. 8, in column 6 in the same line as SEQ ID NO. 11471 is depicted, mitochondrial is mentioned; which is operable linked, too.

In another embodiment thereof the expression cassette does not comprise an additional transit peptide in case in Table I, application no. 8, in column 6 in the same line as SEQ ID NO. 11471 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 55385, or a homolog or fragment thereof, or a homolog thereof being depicted in Table I, application no. 8, column 8, in the same line as SEQ ID NO. 55385, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 55385 is depicted, plastidic is mentioned, or a mitochondrial transit peptide in case in Table I, application no. 8, in column 6 in the same line as SEQ ID NO. 55385 is depicted, mitochondrial is mentioned; which is operable linked, too.

In another embodiment thereof the expression cassette does not comprise an additional transit peptide in case in Table I, application no. 8, in column 6 in the same line as SEQ ID NO. 55385 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 11990, or a homolog or fragment thereof, or a homolog thereof being depicted in Table I, application no. 8, column 8, in the same line as SEQ ID NO. 11990, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 11990 is depicted, plastidic is mentioned, or a mitochondrial transit peptide in case in Table I, application no. 8, in column 6 in the same line as SEQ ID NO. 11990 is depicted, mitochondrial is mentioned; which is operable linked, too.

In another embodiment thereof the expression cassette does not comprise an additional transit peptide in case in Table I, application no. 8, in column 6 in the same line as SEQ ID NO. 11990 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 87655, or a homolog or fragment thereof, or a homolog thereof being depicted in Table I, application no. 8, column 8, in the same line as SEQ ID NO. 87655, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 87655 is depicted, plastidic is mentioned, or a mitochondrial transit peptide in case in Table I, application no. 8, in column 6 in the same line as SEQ ID NO. 87655 is depicted, mitochondrial is mentioned; which is operable linked, too.

In another embodiment thereof the expression cassette does not comprise an additional transit peptide in case in Table I, application no. 8, in column 6 in the same line as SEQ ID NO. 87655 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 116341, or a homolog or fragment thereof, or a homolog thereof being depicted in Table I, application no. 8, column 8, in the same line as SEQ ID NO. 116341, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 116341 is depicted, plastidic is mentioned, or a mitochondrial transit peptide in case in Table I, application no. 8, in column 6 in the same line as SEQ ID NO. 116341 is depicted, mitochondrial is mentioned; which is operable linked, too.

In another embodiment thereof the expression cassette does not comprise an additional transit peptide in case in Table I, application no. 8, in column 6 in the same line as SEQ ID NO. 116341 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 116460, or a homolog or fragment thereof, or a homolog thereof being depicted in Table I, application no. 8, column 8, in the same line as SEQ ID NO. 116460, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 116460 is depicted, plastidic is mentioned, or a mitochondrial transit peptide in case in Table I, application no. 8, in column 6 in the same line as SEQ ID NO. 116460 is depicted, mitochondrial is mentioned; which is operable linked, too.

In another embodiment thereof the expression cassette does not comprise an additional transit peptide in case in Table I, application no. 8, in column 6 in the same line as SEQ ID NO. 116460 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 58058, or a homolog or fragment thereof, or a homolog thereof being depicted in Table I, application no. 8, column 8, in the same line as SEQ ID NO. 58058, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 58058 is depicted, plastidic is mentioned, or a mitochondrial transit peptide in case in Table I, application no. 8, in column 6 in the same line as SEQ ID NO. 58058 is depicted, mitochondrial is mentioned; which is operable linked, too.

In another embodiment thereof the expression cassette does not comprise an additional transit peptide in case in Table I, application no. 8, in column 6 in the same line as SEQ ID NO. 58058 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 58324, or a homolog or fragment thereof, or a homolog thereof being depicted in Table I, application no. 8, column 8, in the same line as SEQ ID NO. 58324, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 58324 is depicted, plastidic is mentioned, or a mitochondrial transit peptide in case in Table I, application no. 8, in column 6 in the same line as SEQ ID NO. 58324 is depicted, mitochondrial is mentioned; which is operable linked, too.

In another embodiment thereof the expression cassette does not comprise an additional transit peptide in case in Table I, application no. 8, in column 6 in the same line as SEQ ID NO. 58324 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 116713, or a homolog or fragment thereof, or a homolog thereof being depicted in Table I, application no. 8, column 8, in the same line as SEQ ID NO. 116713, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 116713 is depicted, plastidic is mentioned, or a mitochondrial transit peptide in case in Table I, application no. 8, in column 6 in the same line as SEQ ID NO. 116713 is depicted, mitochondrial is mentioned; which is operable linked, too.

In another embodiment thereof the expression cassette does not comprise an additional transit peptide in case in Table I, application no. 8, in column 6 in the same line as SEQ ID NO. 116713 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12341, or a homolog or fragment thereof, or a homolog thereof being depicted in Table I, application no. 8, column 8, in the same line as SEQ ID NO. 12341, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 12341 is depicted, plastidic is mentioned, or a mitochondrial transit peptide in case in Table I, application no. 8, in column 6 in the same line as SEQ ID NO. 12341 is depicted, mitochondrial is mentioned; which is operable linked, too.

In another embodiment thereof the expression cassette does not comprise an additional transit peptide in case in Table I, application no. 8, in column 6 in the same line as SEQ ID NO. 12341 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 116848, or a homolog or fragment thereof, or a homolog thereof being depicted in Table I, application no. 8, column 8, in the same line as SEQ ID NO. 116848, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 116848 is depicted, plastidic is mentioned, or a mitochondrial transit peptide in case in Table I, application no. 8, in column 6 in the same line as SEQ ID NO. 116848 is depicted, mitochondrial is mentioned; which is operable linked, too.

In another embodiment thereof the expression cassette does not comprise an additional transit peptide in case in Table I, application no. 8, in column 6 in the same line as SEQ ID NO. 116848 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 61553, or a homolog or fragment thereof, or a homolog thereof being depicted in Table I, application no. 8, column 8, in the same line as SEQ ID NO. 61553, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 61553 is depicted, plastidic is mentioned, or a mitochondrial transit peptide in case in Table I, application no. 8, in column 6 in the same line as SEQ ID NO. 61553 is depicted, mitochondrial is mentioned; which is operable linked, too.

In another embodiment thereof the expression cassette does not comprise an additional transit peptide in case in Table I, application no. 8, in column 6 in the same line as SEQ ID NO. 61553 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 61723, or a homolog or fragment thereof, or a homolog thereof being depicted in Table I, application no. 8, column 8, in the same line as SEQ ID NO. 61723, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 61723 is depicted, plastidic is mentioned, or a mitochondrial transit peptide in case in Table I, application no. 8, in column 6 in the same line as SEQ ID NO. 61723 is depicted, mitochondrial is mentioned; which is operable linked, too.

In another embodiment thereof the expression cassette does not comprise an additional transit peptide in case in Table I, application no. 8, in column 6 in the same line as SEQ ID NO. 61723 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 116948, or a homolog or fragment thereof, or a homolog thereof being depicted in Table I, application no. 8, column 8, in the same line as SEQ ID NO. 116948, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 116948 is depicted, plastidic is mentioned, or a mitochondrial transit peptide in case in Table I, application no. 8, in column 6 in the same line as SEQ ID NO. 116948 is depicted, mitochondrial is mentioned; which is operable linked, too.

In another embodiment thereof the expression cassette does not comprise an additional transit peptide in case in Table I, application no. 8, in column 6 in the same line as SEQ ID NO. 116948 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 14275, or a homolog or fragment thereof, or a homolog thereof being depicted in Table I, application no. 8, column 8, in the same line as SEQ ID NO. 14275, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 14275 is depicted, plastidic is mentioned, or a mitochondrial transit peptide in case in Table I, application no. 8, in column 6 in the same line as SEQ ID NO. 14275 is depicted, mitochondrial is mentioned; which is operable linked, too.

In another embodiment thereof the expression cassette does not comprise an additional transit peptide in case in Table I, application no. 8, in column 6 in the same line as SEQ ID NO. 14275 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 117352, or a homolog or fragment thereof, or a homolog thereof being depicted in Table I, application no. 8, column 8, in the same line as SEQ ID NO. 117352, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 117352 is depicted, plastidic is mentioned, or a mitochondrial transit peptide in case in Table I, application no. 8, in column 6 in the same line as SEQ ID NO. 117352 is depicted, mitochondrial is mentioned; which is operable linked, too.

In another embodiment thereof the expression cassette does not comprise an additional transit peptide in case in Table I, application no. 8, in column 6 in the same line as SEQ ID NO. 117352 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 90165, or a homolog or fragment thereof, or a homolog thereof being depicted in Table I, application no. 8, column 8, in the same line as SEQ ID NO. 90165, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 90165 is depicted, plastidic is mentioned, or a mitochondrial transit peptide in case in Table I, application no. 8, in column 6 in the same line as SEQ ID NO. 90165 is depicted, mitochondrial is mentioned; which is operable linked, too.

In another embodiment thereof the expression cassette does not comprise an additional transit peptide in case in Table I, application no. 8, in column 6 in the same line as SEQ ID NO. 90165 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 117482, or a homolog or fragment thereof, or a homolog thereof being depicted in Table I, application no. 8, column 8, in the same line as SEQ ID NO. 117482, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 117482 is depicted, plastidic is mentioned, or a mitochondrial transit peptide in case in Table I, application no. 8, in column 6 in the same line as SEQ ID NO. 117482 is depicted, mitochondrial is mentioned; which is operable linked, too.

In another embodiment thereof the expression cassette does not comprise an additional transit peptide in case in Table I, application no. 8, in column 6 in the same line as SEQ ID NO. 117482 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 64964, or a homolog or fragment thereof, or a homolog thereof being depicted in Table I, application no. 8, column 8, in the same line as SEQ ID NO. 64964, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 64964 is depicted, plastidic is mentioned, or a mitochondrial transit peptide in case in Table I, application no. 8, in column 6 in the same line as SEQ ID NO. 64964 is depicted, mitochondrial is mentioned; which is operable linked, too.

In another embodiment thereof the expression cassette does not comprise an additional transit peptide in case in Table I, application no. 8, in column 6 in the same line as SEQ ID NO. 64964 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 66772, or a homolog or fragment thereof, or a homolog thereof being depicted in Table I, application no. 8, column 8, in the same line as SEQ ID NO. 66772, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 66772 is depicted, plastidic is mentioned, or a mitochondrial transit peptide in case in Table I, application no. 8, in column 6 in the same line as SEQ ID NO. 66772 is depicted, mitochondrial is mentioned; which is operable linked, too.

In another embodiment thereof the expression cassette does not comprise an additional transit peptide in case in Table I, application no. 8, in column 6 in the same line as SEQ ID NO. 66772 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 117495, or a homolog or fragment thereof, or a homolog thereof being depicted in Table I, application no. 8, column 8, in the same line as SEQ ID NO. 117495, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 117495 is depicted, plastidic is mentioned, or a mitochondrial transit peptide in case in Table I, application no. 8, in column 6 in the same line as SEQ ID NO. 117495 is depicted, mitochondrial is mentioned; which is operable linked, too.

In another embodiment thereof the expression cassette does not comprise an additional transit peptide in case in Table I, application no. 8, in column 6 in the same line as SEQ ID NO. 117495 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 118212, or a homolog or fragment thereof, or a homolog thereof being depicted in Table I, application no. 8, column 8, in the same line as SEQ ID NO. 118212, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 118212 is depicted, plastidic is mentioned, or a mitochondrial transit peptide in case in Table I, application no. 8, in column 6 in the same line as SEQ ID NO. 118212 is depicted, mitochondrial is mentioned; which is operable linked, too.

In another embodiment thereof the expression cassette does not comprise an additional transit peptide in case in Table I, application no. 8, in column 6 in the same line as SEQ ID NO. 118212 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 16883, or a homolog or fragment thereof, or a homolog thereof being depicted in Table I, application no. 8, column 8, in the same line as SEQ ID NO. 16883, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 8, in column 6 in the same line as SEQ ID NO. 16883 is depicted, plastidic is mentioned, or a mitochondrial transit peptide in case in Table I, application no. 8, in column 6 in the same line as SEQ ID NO. 16883 is depicted, mitochondrial is mentioned; which is operable linked, too.

In another embodiment thereof the expression cassette does not comprise an additional transit peptide in case in Table I, application no. 8, in column 6 in the same line as SEQ ID NO. 16883 is depicted, non-targeted is mentioned.

Accordingly, in one embodiment, the process according to the invention comprises the following steps:
(a) introducing of a nucleic acid construct comprising the nucleic acid molecule of the invention (and if desired, in combination with the nucleic acid encoding a respective transit peptide) or used in the process of the invention or encoding the polypeptide of the present invention or used in the process of the invention; or
(b) introducing of a nucleic acid molecule, including regulatory sequences and/or factors (and if desired, in combination with the nucleic acid encoding a respective transit peptide), which expression increases the expression of the nucleic acid molecule of the invention or used in the process of the invention or encoding the polypeptide of the present invention or used in the process of the invention; in a cell, or a non-human organism or a part thereof, preferably in a plant, plant cell or a microorganism, preferably in the organelles such as the plastids or mitochondria thereof, or preferably non-targeted, or
(c) introducing an expression cassette as mentioned above, and
(d) expressing of the gene product encoded by the nucleic acid construct or the nucleic acid molecule (and if desired, in combination with the nucleic acid encoding a respective transit peptide) or expression cassette mentioned under (a), (b) or (c) in the cell or the non-human organism or part thereof.

For expression in a host organism, for example a plant, the expression cassette is advantageously inserted into a vector such as by way of example a plasmid, a phage or other DNA which allows optimal expression of the genes in the host organism. Examples of suitable plasmids are: in E. coli pLG338, pACYC184, pBR series such as e.g. pBR322, pUC series such as pUC18 or pUC19, M113mp series, pKC30, pRep4, pHS1, pHS2, pPLc236, pMBL24, μLG200, pUR290, pIN-111113-B1, λgt11 or pBdCI; in Streptomyces pIJ101, pIJ364, pIJ702 or pIJ361; in Bacillus pUB110, pC194 or pBD214; in Corynebacterium pSA77 or pAJ667; in fungi pALS1, pl L2 or pBB116; other advantageous fungal vectors are described by Romanos M. A. et al., Yeast 8, 423 (1992) and by van den Hondel C. A. M. J. J. et al. [(1991) “Heterologous gene expression in filamentous fungi”] as well as in “More Gene Manipulations” in “Fungi” in Bennet J. W. & Lasure L. L., eds., pp. 396-428, Academic Press, San Diego, and in “Gene transfer systems and vector development for filamentous fungi” [van den Hondel C. A. M. J. J. & Punt P. J. (1991) in: Applied Molecular Genetics of Fungi, Peberdy J. F. et al., eds., pp. 1-28, Cambridge University Press: Cambridge]. Examples of advantageous yeast vectors are 2pM, pAG-1, YEp6, YEp13 or pEMBLYe23. Examples of algal or plant plasmids are pLGV23, pGHlac+, pBIN19, pAK2004, pVKH or pDH51 (see Schmidt R., Willmitzer L., Plant Cell Rep. 7, 583 (1988)). The vectors identified above or derivatives of the vectors identified above are a small selection of the possible plasmids. Further plasmids are well known to those skilled in the art and may be found, for example, in “Cloning Vectors” (Eds. P. H. Pouwels et al., Elsevier, Amsterdam-New York-Oxford, 1985, ISBN 0 444 904018). Suitable plant vectors are described inter alia in “Methods in Plant Molecular Biology and Biotechnology” (CRC Press, Ch. 6/7, pp. 71-119). Advantageous vectors are known as shuttle vectors or binary vectors which replicate in E. coli and Agrobacterium. Examples for binary vectors are pBIN19, pBI101, pBinAR, pGPTV, pCAMBIA, pBIB-HYG, pBecks, pGreen or pPZP (Hajukiewicz P. et al., Plant Mol. Biol. 25, 989 (1994), and Hellens et al, Trends in Plant Science 5, 446 (2000)).

In a further embodiment of the vector the expression cassette according to the invention may also advantageously be introduced into the organisms in the form of a linear DNA and be integrated into the genome of the host organism by way of heterologous or homologous recombination. This linear DNA may be composed of a linearized plasmid or only of the expression cassette as vector or the respective nucleic acid sequences (if desired, in combination with the nucleic acid encoding a respective transit peptide) according to the invention.

In a further advantageous embodiment the nucleic acid sequence (if desired, in combination with the nucleic acid encoding a respective transit peptide) according to the invention can also be introduced into an organism on its own.

If in addition to the nucleic acid sequence according to the invention (and if desired, in combination with the nucleic acid encoding a respective transit peptide) further genes are to be introduced into the organism, all genes altogether with a marker in a single vector or each single gene with a marker in a vector can be introduced into the organism, whereby the different vectors can be introduced simultaneously or successively.

The vector advantageously contains at least one copy of the nucleic acid sequences according to the invention (and if desired, in combination with the nucleic acid encoding a respective transit peptide) or of the expression cassette (=gene construct) according to the invention.

The invention further provides a vector, comprising a nucleic acid molecule comprising 1) a nucleic acid molecule selected from the group consisting of:
(a) a nucleic acid molecule encoding a polypeptide shown in Table II, column 5 or 8, application no. 8, preferably shown in Table II A, application no. 8, in column 5, or in Table II A, application no. 8, column 8, or in Table II B, application no. 8, column 8, or a homolog or a fragment thereof;
(b) a nucleic acid molecule shown in Table I, application no. 8, column 5 or 8, preferably shown in Table I A, application no. 8, in column 5, or in Table I A, application no. 8, column 8, or in Table I B, application no. 8, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 8, preferably shown in Table II A, application no. 8, in column 5, or in Table II A, application no. 8, column 8, or in Table II B, application no. 8, column 8;
(d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 8, preferably shown in Table I A, application no. 8, in column 5, or in Table I A, application no. 8, column 8, or in Table I B, application no. 8, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 8;
(i) a nucleic acid molecule encoding a polypeptide having the activity represented by a protein as depicted in column 5 of Table II, application no. 8;
(j) a nucleic acid molecule comprising a polynucleotide which is obtained by amplifying a cDNA library or a genomic library using the primers in Table III, application no. 8, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 8.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 8, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 8.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 8, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 8, in the same line as of the respective nucleic acid molecule according to 1) “plastidic” is depicted, or encoding a mitochondrial transit peptide, especially in case in column 6 of Table I, application no. 8, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 8, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 8, in the same line as of the respective nucleic acid molecule according to 1) “plastidic” is depicted, or encoding a mitochondrial transit peptide, especially in case in column 6 of Table I, application no. 8, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operable linked; a nucleic acid molecule encoding a transit peptide, especially in case in column 6 of Table I, application no. 8, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 8, in the same line as of the respective nucleic acid molecule according to 1) “plastidic” is depicted, or encoding a mitochondrial transit peptide, especially in case in column 6 of Table I, application no. 8, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 8 (in the same line).

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 8, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 8.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 8, in the same line as of the respective nucleic acid molecule according to 1) “plastidic” is depicted, or encoding a mitochondrial transit peptide, especially in case in column 6 of Table I, application no. 8, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 8, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 8, in the same line as of the respective nucleic acid molecule according to 1) “plastidic” is depicted, or encoding a mitochondrial transit peptide, especially in case in column 6 of Table I, application no. 8, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 8 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 8, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 8 (in the same line) and confers a generation or an increase of the respective fine chemical in a non-human organism or a part thereof as compared to a corresponding wild-type non-human organism or part thereof.

In a preferred embodiment of said vector said nucleic acid molecule according to 1 (a), (b), (c), (d), (e), (f), (g), (h), (i), (j) or (k), said promoter and optionally said nucleic acid molecule encoding a transit peptide are encompassed in an expression cassette. Accordingly in an embodiment the invention provides a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

The invention further provides a vector comprising a nucleic acid molecule comprising 1) a nucleic acid molecule selected from the group consisting of:
(a) a nucleic acid molecule encoding a polypeptide shown in Table II, column 5 or 8, application no. 8, preferably shown in Table II A, application no. 8, in column 5, or in Table II A, application no. 8, column 8, or in Table II B, application no. 8, column 8, or a homolog or a fragment thereof;
(b) a nucleic acid molecule shown in Table I, application no. 8, column 5 or 8, preferably shown in Table I A, application no. 8, in column 5, or in Table I A, application no. 8, column 8, or in Table I B, application no. 8, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 8, preferably shown in Table II A, application no. 8, in column 5, or in Table II A, application no. 8, column 8, or in Table II B, application no. 8, column 8;
(d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 8, preferably shown in Table I A, application no. 8, in column 5, or in Table I A, application no. 8, column 8, or in Table I B, application no. 8, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 8;
(i) a nucleic acid molecule encoding a polypeptide having the activity represented by a protein as depicted in column 5 of Table II, application no. 8;
(j) a nucleic acid molecule comprising a polynucleotide which is obtained by amplifying a cDNA library or a genomic library using the primers in Table III, application no. 8, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto;
   whereby, preferably, the nucleic acid molecule according to 1 (a), (b), (c), (d), (e), (f), (g), (h), (i), (j) or (k) distinguishes over the sequence depicted in Table I A and/or I B, application no. 8, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 8, column 5 or 8, or the coding region thereof, respectively. In another embodiment, the nucleic acid molecule of the present invention, preferably the coding region thereof, is 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 8, column 5 or 8, or the coding region thereof, respectively. In a further embodiment the nucleic acid molecule does not encode the polypeptide sequence shown in Table II A and/or II B, application no. 8, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 8, column 5 or 8 and does not encode a protein of the sequence shown in Table II A and/or II B, application no. 8, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 8, column 5 and 8, or the coding region thereof. In a further embodiment, the protein of the present invention is at least 30%,40%, 50%, 60%, 70%, 80%, 90%, 95% identical to protein sequence depicted in Table II A and/or II B, application no. 8, column 5 or 8 but less than 100%, preferably less than 99.999%, 99.99% or 99.9%, more preferably less than 99%, 985, 97%, 96% or 95% identical to the sequence shown in Table II A and/or II B, application no. 8, column 5 or 8.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 8.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 8, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 8.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 8, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 8, in the same line as of the respective nucleic acid molecule according to 1) “plastidic” is depicted, or encoding a mitochondrial transit peptide, especially in case in column 6 of Table I, application no. 8, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 8, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 8, in the same line as of the respective nucleic acid molecule according to 1) “plastidic” is depicted, or encoding a mitochondrial transit peptide, especially in case in column 6 of Table I, application no. 8, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 8, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 8, in the same line as of the respective nucleic acid molecule according to 1) “plastidic” is depicted, or encoding a mitochondrial transit peptide, especially in case in column 6 of Table I, application no. 8, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 8.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 8, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 8.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 8, in the same line as of the respective nucleic acid molecule according to 1) “plastidic” is depicted, or encoding a mitochondrial transit peptide, especially in case in column 6 of Table I, application no. 8, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 8, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 8, in the same line as of the respective nucleic acid molecule according to 1) “plastidic” is depicted, or encoding a mitochondrial transit peptide, especially in case in column 6 of Table I, application no. 8, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 8 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 8, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 8 and confers a generation or an increase of the respective fine chemical in a non-human organism or a part thereof as compared to a corresponding wild-type non-human organism or part thereof.

In a preferred embodiment of said vector said nucleic acid molecule according to 1 (a), (b), (c), (d), (e), (f), (g), (h), (i), (j) or (k), said promoter and optionally said nucleic acid molecule encoding a transit peptide are encompassed in an expression cassette. Accordingly in an embodiment the invention provides a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

The invention further provides a vector, especially an expression vector, comprising a FCRP encoding nucleic acid as described above, wherein expression of the vector or FCRP encoding nucleic acid, respectively in a host cell results in the production of a the respective fine chemical, preferably in an increased production of the respective fine chemical, as compared to a wild type of the host cell.

As used herein, the term “vector” refers to a nucleic acid molecule capable of transporting another nucleic acid which has been linked thereto. One type of vector is a “plasmid”, which refers to a circular double stranded DNA loop into which additional DNA segments can be ligated. Another type of vector is a viral vector, wherein additional DNA segments can be ligated into the viral genome. Further types of vectors can be linearized nucleic acid sequences, such as transposons, which are pieces of DNA which can copy and insert themselves. There have been two types of transposons found: simple transposons, known as Insertion Sequences and composite transposons, which can have several genes as well as the genes that are required for transposition. Certain vectors are capable of autonomous replication in a host cell into which they are introduced (e.g. bacterial vectors having a bacterial origin of replication and episomal mammalian vectors). Other vectors (e.g. non-episomal mammalian vectors) are integrated into the genome of a host cell or an organelle upon introduction into the host cell, and thereby are replicated along with the host or organelle genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include other forms of expression vectors, such as viral vectors (e.g., replication defective retroviruses, adenoviruses, and adeno-associated viruses), which exert similar functions.

The term vector furthermore encompasses other vectors known to those skilled in the art such as by way of example phages, viruses such as SV40, CMV, baculovirus, adenovirus, transposons, IS elements, phasmids, phagemids, cosmids, linear or circular DNA. These vectors can be replicated autonomously in the host organism or be chromosomally replicated, chromosomal replication being preferred.

The recombinant expression vectors of the invention comprise a nucleic acid molecule of the invention in a form suitable for expression of the nucleic acid molecule in a host cell, which means that the recombinant expression vectors include one or more regulatory sequences, selected on the basis of the host cells to be used for expression, and, if desired a targeting sequence, which are operatively linked to the nucleic acid sequence to be expressed. As used herein with respect to a recombinant expression vector, “operatively linked” is intended to mean that the nucleotide sequence of interest is linked to the regulatory sequence(s)/targeting sequence in a manner which allows for expression of the nucleotide sequence in such way that the predicted function assigned to the nucleic sequence is fulfilled (e.g. in an in vitro transcription/translation system or in a host cell when the vector is introduced into the host cell). The term “regulatory sequence” is intended to include promoters, enhancers, and other expression control elements (e.g. polyadenylation signals). Such regulatory sequences are described, for example, in Goeddel “Gene Expression Technology: Methods in Enzymology 185”, Academic Press, San Diego, Calif. (1990), and Gruber and Crosby, in: Methods in Plant Molecular Biology and Biotechnology, eds. Glick and Thompson, Chapter 7, 89-108, CRC Press; Boca Raton, Fla., including the references therein. Regulatory sequences include those that direct constitutive expression of a nucleotide sequence in many types of host cells and those that direct expression of the nucleotide sequence only in certain host cells or under certain conditions. It will be appreciated by those skilled in the art that the design of the expression vector can depend on such factors as the choice of the host cell to be transformed, the level of expression of polypeptide desired, etc. The term “regulatory sequence” is to be considered as being encompassed by the term “regulatory signal”. The expression vectors of the invention can be introduced into host cells to thereby produce polypeptides or peptides, including fusion polypeptides or peptides, encoded by nucleic acid molecules as described herein (e.g., fusion polypeptides, FCRPs etc.).

In another embodiment the recombinant expression vectors of the invention can be designed specifically for expression of the polypeptides of the invention in plant cells.

For example, FCRP genes can be expressed in plant cells (see R. Schmidt, L. Willmitzer, Plant Cell Rep. 7, 583 (1988); Plant Molecular Biology and Biotechnology, C Press, Boca Raton, Fla., Chapter 6/7, p. 71-119 (1993); F. F. White, B. Jenes et al., Techniques for Gene Transfer, in: Transgenic Plants, Vol. 1, Engineering and Utilization, eds. Kung and Wu R., 128-43, Academic Press: 1993; Potrykus, Annu. Rev. Plant Physiol. Plant Molec. Biol. 42, 205 (1991) and references cited therein). Suitable host cells are discussed further in Goeddel, “Gene Expression Technology: Methods in Enzymology 185”, Academic Press: San Diego, Calif. (1990). By way of example the plant expression cassette can be installed in the pRT transformation vector ((a) Toepfer et al., Methods Enzymol. 217, 66 (1993), (b) Toepfer et al., Nucl. Acids. Res. 15, 5890 (1987)). Alternatively, the recombinant expression vector can be transcribed and translated in vitro, for example using T7 promoter regulatory sequences and T7 polymerase.

Expression vectors employed in prokaryotes frequently make use of inducible systems with and without fusion proteins or fusion oligopeptides, wherein these fusions can ensue in both N-terminal and C-terminal manner or in other useful domains of a protein. Such fusion vectors usually have the following purposes: 1) to increase the RNA expression rate; 2) to increase the achievable protein synthesis rate; 3) to increase the solubility of the protein; 4) or to simplify purification by means of a binding sequence usable for affinity chromatography. Proteolytic cleavage points are also frequently introduced via fusion proteins, which allow cleavage of a portion of the fusion protein and purification. Such recognition sequences for proteases are recognized, e.g. factor Xa, thrombin and enterokinase.

Typical advantageous fusion and expression vectors are pGEX (Pharmacia Biotech Inc; D. B. Smith and K. S. Johnson, Gene 67, 31 (1988)), pMAL (New England Biolabs, Beverly, Mass.) and pRIT5 (Pharmacia, Piscataway, N.J.) which contains glutathione S-transferase (GST), maltose binding protein or protein A.

In an embodiment of the present invention, the coding sequence for the polypeptide of the invention is cloned into a pGEX expression vector to create a vector encoding a fusion polypeptide comprising, from the N-terminus to the C-terminus, GST-thrombin cleavage site-X polypeptide. The fusion polypeptide can be purified by affinity chromatography using glutathione-agarose resin. Recombinant FCRP unfused to GST can be recovered by cleavage of the fusion polypeptide with thrombin. Other examples of E. coli expression vectors are pTrc (Amann et al., Gene 69, 301 (1988)) and pET vectors (Studier et al., “Gene Expression Technology: Methods in Enzymology 185”, Academic Press, San Diego, Calif. (1990) 60-89; Stratagene, Amsterdam, The Netherlands).

Target gene expression from the pTrc vector relies on host RNA polymerase transcription from a hybrid trp-lac fusion promoter. Target gene expression from the pET 11d vector relies on transcription from a T7 gn10-lac fusion promoter mediated by a co-expressed viral RNA polymerase (T7 gn1). This viral polymerase is supplied by host strains BL21(DE3) or HMS174(DE3) from a resident I prophage harboring a T7 gn1 gene under the transcriptional control of the lacUV 5 promoter.

In a further embodiment of the present invention, the FCRPs are expressed in plants and plant cells such as unicellular plant cells (e.g. algae) (see Falciatore et al., Marine Biotechnology 1 (3), 239 (1999) and references therein) and plant cells from higher plants (e.g., the spermatophytes, such as crop plants), for example to regenerate plants from the plant cells. A nucleic acid molecule coding for FCRP as depicted in Table II, column 5 or 8, or a homolog or a fragment thereof, (and, if desired, in combination with the nucleic acid encoding a respective transit peptide) may be “introduced” into a plant cell by any means, including transfection, transformation or transduction, electroporation, particle bombardment, agroinfection, and the like.

One transformation method known to those of skill in the art is the dipping of a flowering plant into an Agrobacteria solution, wherein the Agrobacteria contains the nucleic acid of the invention, followed by breeding of the transformed gametes.

Other suitable methods for transforming or transfecting host cells including plant cells can be found in Sambrook et al., “Molecular Cloning: A Laboratory Manual”. 2nd ed., Cold Spring Harbor Laboratory, Cold Spring Harbor Laboratory Press, Cold Spring Harbor, N.Y., 1989, and other laboratory manuals such as “Methods in Molecular Biology”, 1995, Vol. 44, Agrobacterium protocols, ed: Gartland and Davey, Humana Press, Totowa, New Jersey. As generated or increased production of the fine chemical is generally wished to be inherited into a wide variety of monocotyledonous or dicotyledonous plants, preferably plants like maize, wheat, rye, oat, triticale, rice, barley, sugar cane, soybean, peanut, cotton, rapeseed and canola, manihot, pepper, sunflower and tagetes, sugar beet, solanaceous plants like potato, tobacco, eggplant, and tomato, Vicia species, pea, alfalfa, bushy plants (coffee, cacao, tea), Salix species, trees (oil palm, coconut), perennial grasses, and forage crops, these crop plants are also preferred target plants for a genetic engineering as one further embodiment of the present invention. Forage crops include, but are not limited to Wheatgrass, Canarygrass, Bromegrass, Wildrye Grass, Bluegrass, Orchardgrass, Alfalfa, Salfoin, Birdsfoot Trefoil, Alsike Clover, Red Clover and Sweet Clover.

In one embodiment of the present invention, transfection of a nucleic acid molecule coding for FCRP e.g. as depicted in Table II column 5 or 8, or a homolog or a fragment thereof, (and, if desired, in combination with the nucleic acid encoding a respective transit peptide) into a plant is achieved by Agrobacterium mediated gene transfer. Agrobacterium mediated plant transformation can be performed using for example the GV3101(pMP90) (Koncz and Schell, Mol. Gen. Genet. 204, 383 (1986)) or LBA4404 (Clontech) Agrobacterium tumefaciens strain. Transformation can be performed by standard transformation and regeneration techniques (Deblaere et al., Nucl. Acids Res. 13, 4777 (1994), Gelvin, B. Stanton and Robert A. Schilperoort, Plant Molecular Biology Manual, 2nd ed.—Dordrecht : Kluwer Academic Publ., 1995.—in Sect., Ringbuc Zentrale Signatur: BT11-P ISBN 0-7923-2731-4; Bernard R. Glick., John E. Thompson, “Methods in Plant Molecular Biology and Biotechnology”, Boca Raton: CRC Press, 1993 p. 360, ISBN 0-8493-5164-2). For example, rapeseed can be transformed via cotyledon or hypocotyl transformation (Moloney et al., Plant Cell Report 8, 238 (1989); De Block et al., Plant Physiol. 91, 694 (1989)). Use of antibiotics for Agrobacterium and plant selection depends on the binary vector and the Agrobacterium strain used for transformation. Rapeseed selection is normally performed using kanamycin as selectable plant marker. Agrobacterium mediated gene transfer to flax can be performed using, for example, a technique described by Mlynarova et al., Plant Cell Report 13, 282 (1994). Additionally, transformation of soybean can be performed using for example a technique described in European Patent No. 424 047, U.S. Pat. No. 5,322,783, European Patent No. 397 687, U.S. Pat. No. 5,376,543 or U.S. Pat. No. 5,169,770. Transformation of maize can be achieved by particle bombardment, polyethylene glycol mediated DNA uptake or via the silicon carbide fiber technique. (See, for example, Freeling and Walbot “The maize handbook” Springer Verlag: New York (1993) ISBN 3-540 -97826-7). A specific example of maize transformation is found in U.S. Pat. No. 5,990,387, and a specific example of wheat transformation can be found in PCT Application No. WO 93/07256. Examples for rice transformation can be found e.g. in EP 1 728 418 or EP 897 013.

According to the present invention, the introduced nucleic acid molecule coding for FCRP e.g. as depicted in Table II, column 5 or 8, or a homolog or a fragment thereof, may be maintained in the plant cell stably if it is incorporated into a non-chromosomal autonomous replicon or integrated into the plant chromosomes or organelle genome. Alternatively, the introduced FCRP may be present on an extra-chromosomal non-replicating vector and be transiently expressed or transiently active.

Whether present in an extra-chromosomal non-replicating vector or a vector that is integrated into a chromosome, the nucleic acid molecule coding for FCRP as depicted in Table II, column 5 or 8, a homolog or a fragment thereof, preferably resides in a non-human organism expression cassette, preferably a microorganism expression cassette or a plant expression cassette. A plant expression cassette preferably contains regulatory sequences capable of driving gene expression in plant cells that are operatively linked so that each sequence can fulfill its function, for example, termination of transcription by polyadenylation signals. Preferred polyadenylation signals are those originating from Agrobacterium tumefaciens t-DNA such as the gene 3 known as octopine synthase of the Ti-plasmid pTiACH5 (Gielen et al., EMBO J. 3, 835 (1984)) or functional equivalents thereof but also all other terminators functionally active in plants are suitable. As plant gene expression is very often not limited on transcriptional levels, a plant expression cassette preferably contains other operatively linked sequences like translational enhancers such as the overdrive-sequence containing the 5″-untranslated leader sequence from tobacco mosaic virus enhancing the polypeptide per RNA ratio (Gallie et al., Nucl. Acids Research 15, 8693 (1987)). Examples of plant expression vectors include those detailed in: D. Becker et al., Plant Mol. Biol. 20, 1195 (1992); and M. W. Bevan, Nucl. Acid. Res. 12, 8711 (1984); and “Vectors for Gene Transfer in Higher Plants” in: Transgenic Plants, Vol. 1, Engineering and Utilization, eds. Kung and Wu R., Academic Press, 1993, S. 15-38. “Transformation” is defined herein as a process for introducing DNA into a non-human organism, preferably a microorganism or a plant or a part thereof, like a plant cell or plant tissue. In an embodiment the transformation is performed with heterologous DNA. In another embodiment the transformation is performed with “additional” homologous DNA. Transformation may occur under natural or artificial conditions using various methods well known in the art. Transformation may rely on any known method for the insertion of (foreign, additional) nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome. They also include cells which transiently express the inserted DNA or RNA for limited periods of time. Transformed plant cells, plant tissue, or plants are understood to encompass not only the end product of a transformation process, but also transgenic progeny thereof.

The terms “transformed,” “transgenic,” and “recombinant” refer to a host organism such as a microorganism, e.g. a bacterium, or a plant into which a nucleic acid molecule has been introduced. In an embodiment the introduced nucleic acid molecule is heterogenous. In another embodiment the introduced DNA is homologous. The nucleic acid molecule can be stably integrated into the genome of the host or the nucleic acid molecule can also be present as an extra-chromosomal molecule. Such an extra-chromosomal molecule can be autoreplicating. A “non-transformed”, “non-transgenic” or “non-recombinant” host refers to a wild-type organism, e.g. a bacterium or plant, which does not contain the heterologous nucleic acid molecule or the additional homologous nucleic acid molecule.

In an embodiment a “transgenic plant”, as used herein, refers to a plant which contains a nucleotide sequence inserted into either its nuclear genome or organelle genome. In an embodiment the introduced nucleic acid molecule is heterogenous. In another embodiment the introduced DNA is homologous. In each case it encompasses further the offspring generations i.e. the Ti-, T2- and consecutively generations or BC1-, BC2- and consecutively generation as well as crossbreeds thereof with non-transgenic or other transgenic plants, as long as it contains said nucleic acid sequence.

The host organism (=transgenic organism) advantageously contains at least one copy of the nucleic acid in addition according to the invention and/or of the nucleic acid construct in addition according to the invention.

In principle all non-human organism can be used as host organism. In an embodiment the transgenic non-human organism or cell is a prokaryotic organism. In an embodiment the transgenic non-human organism or cell is an eukaryotic organism, like an alga, a non-human animal or a plant, in particular an algae or a plant. Preferred transgenic plants are monocotyledonous or dicotyledonous plants, preferably, for example, selected from the families Aceraceae, Anacardiaceae, Apiaceae, Asteraceae, Brassicaceae, Cactaceae, Cucurbitaceae, Euphorbiaceae, Fabaceae, Malvaceae, Nymphaeaceae, Papaveraceae, Rosaceae, Salicaceae, Solanaceae, Arecaceae, Bromeliaceae, Cyperaceae, lridaceae, Liliaceae, Orchidaceae, Gentianaceae, Labiaceae, Magnoliaceae, Ranunculaceae, Carifolaceae, Rubiaceae, Scrophulariaceae, Caryophyllaceae, Ericaceae, Polygonaceae, Violaceae, Juncaceae or Poaceae and preferably from a plant selected from the group of the families Apiaceae, Asteraceae, Brassicaceae, Cucurbitaceae, Fabaceae, Papaveraceae, Rosaceae, Solanaceae, Liliaceae or Poaceae. Preferred are crop plants such as plants advantageously selected from the group of the genus peanut, oilseed rape, canola, sunflower, safflower, olive, sesame, hazelnut, almond, avocado, bay, pumpkin/squash, linseed, soya, pistachio, borage, maize, wheat, rye, oats, sorghum and millet, triticale, rice, barley, sugarcane, cotton, cassava, potato, sugarbeet, egg plant, alfalfa, and perennial grasses and forage plants, oil palm, vegetables (brassicas, root vegetables, tuber vegetables, pod vegetables, fruiting vegetables, onion vegetables, leafy vegetables and stem vegetables), buckwheat, Jerusalem artichoke, broad bean, vetches, lentil, dwarf bean, lupin, clover and lucerne for mentioning only some of them.

In one embodiment of the invention transgenic plants are selected from the group comprising monocotyledonous or dicotyledonous plants, preferably cereals, soybean, rapeseed (including oil seed rape, especially canola and winter oil seed rape), cotton, sugarcane and potato, especially corn, soy, rapeseed (including oil seed rape, especially canola and winter oil seed rape), cotton, wheat and rice.

In one embodiment, the transgenic plant is a crop plant, in particular a transgenic plant belonging to e.g. Anacardium occidentale, Calendula officinalis, Carthamus tinctorius, Cichorium intybus, Cynara scolymus, Helianthus annus, Tagetes lucida, Tagetes erecta, Tagetes tenuifolia; Daucus carota; Corylus avellana, Corylus colurna, Borago officinalis; Brassica napus, Brassica rapa ssp., Sinapis arvensis Brassica juncea, Brassica juncea var. juncea, Brassica juncea var. crispifolia, Brassica juncea var. foliosa, Brassica nigra, Brassica sinapioides, Melanosinapis communis, Brassica oleracea, Arabidopsis thaliana, Anana comosus, Ananas ananas, Bromelia comosa, Carica papaya, Cannabis sative, Ipomoea batatus, Ipomoea pandurata, Convolvulus batatas, Convolvulus tiliaceus, Ipomoea fastigiata, Ipomoea tiliacea, Ipomoea triloba, Convolvulus panduratus, Beta vulgaris, Beta vulgaris var. altissima, Beta vulgaris var. vulgaris, Beta maritima, Beta vulgaris var. perennis, Beta vulgaris var. conditiva, Beta vulgaris var. esculenta, Cucurbita maxima, Cucurbita mixta, Cucurbita pepo, Cucurbita moschata, Olea europaea, Manihot utilissima, Janipha manihot, Jatropha manihot., Manihot aipil, Manihot dulcis, Manihot manihot, Manihot melanobasis, Manihot esculenta, Ricinus communis, Pisum sativum, Pisum arvense, Pisum humile, Medicago sativa, Medicago falcata, Medicago varia, Glycine max Dolichos soja, Glycine gracilis, Glycine hispida, Phaseolus max, Soja hispida, Glycine max, Cocos nucifera, Pelargonium grossularioides, Oleum cocoas, Laurus nobilis, Persea americana, Arachis hypogaea, Linum usitatissimum, Linum humile, Linum austriacum, Linum bienne, Linum angustifolium, Linum catharticum, Linum flavum, Linum grandiflorum, Adenolinum grandiflorum, Linum lewisii, Linum narbonense, Linum perenne, Linum perenne var. lewisii, Linum pratense, Linum trigynum, Punica granatum, Gossypium hirsutum, Gossypium arboreum, Gossypium barbadense, Gossypium herbaceum, Gossypium thurberi, Musa nana, Musa acuminata, Musa paradisiaca, Musa spp., Elaeis guineensis, Papaver orientale, Papaver rhoeas, Papaver dubium, Sesamum indicum, Piper aduncum, Piper amalago, Piper angustifolium, Piper auritum, Piper betel, Piper cubeba, Piper longum, Piper nigrum, Piper retrofractum, Artanthe adunca, Artanthe elongata, Peperomia elongata, Piper elongatum, Steffensia elongata, Hordeum vulgare, Hordeum jubatum, Hordeum murinum, Hordeum secalinum, Hordeum distichon Hordeum aegiceras, Hordeum hexastichon., Hordeum hexastichum, Hordeum irregulare, Hordeum sativum, Hordeum secalinum, Avena sativa, Avena fatua, Avena byzantina, Avena fatua var. sativa, Avena hybrida, Sorghum bicolor, Sorghum halepense, Sorghum saccharatum, Sorghum vulgare, Andropogon drummondii, Holcus bicolor, Holcus sorghum, Sorghum aethiopicum, Sorghum arundinaceum, Sorghum caffrorum, Sorghum cernuum, Sorghum dochna, Sorghum drummondii, Sorghum durra, Sorghum guineense, Sorghum lanceolatum, Sorghum nervosum, Sorghum saccharatum, Sorghum subglabrescens, Sorghum verticilliflorum, Sorghum vulgare, Holcus halepensis, Sorghum miliaceum millet, Panicum militaceum, Zea mays, Triticum aestivum, Triticum durum, Triticum turgidum, Triticum hybernum, Triticum macha, Triticum sativum or Triticum vulgare, Cofea spp., Coffea arabica, Coffea canephora, Coffea liberica, Capsicum annuum, Capsicum annuum var. glabriusculum, Capsicum frutescens, Capsicum annuum, Nicotiana tabacum, Solanum tuberosum, Solanum melongena, Lycopersicon esculentum, Lycopersicon lycopersicum., Lycopersicon pyriforme, Solanum integrifolium, Solanum lycopersicum Theobroma cacao, Panicum virgatum or Camellia sinensis.

In another embodiment, the transgenic plant belongs to monocotyledonous or dicotyledonous plants, preferably to Anacardiaceae such as the genera Pistacia, Mangifera, Anacardium e.g. the species Pistacia vera [pistachios, Pistazie], Mangifer indica [Mango] or Anacardium occidentale [Cashew]; Asteraceae such as the genera Calendula, Carthamus, Centaurea, Cichorium, Cynara, Helianthus, Lactuca, Locusta, Tagetes, Valeriana e.g. the species Calendula officinalis [Marigold], Carthamus tinctorius [safflower], Centaurea cyanus [cornflower], Cichorium intybus [blue daisy], Cynara scolymus [Artichoke], Helianthus annus [sunflower], Lactuca sativa, Lactuca crispa, Lactuca esculenta, Lactuca scariola L. ssp. sativa, Lactuca scariola L. var. integrate, Lactuca scariola L. var. integrifolia, Lactuca sativa subsp. romana, Locusta communis, Valeriana locusta [lettuce], Tagetes lucida, Tagetes erecta or Tagetes tenuifolia [Marigold]; Apiaceae such as the genera Daucus e.g. the species Daucus carota [carrot]; Betulaceae such as the genera Corylus e.g. the species Corylus avellana or Corylus colurna [hazelnut]; Boraginaceae such as the genera Borago e.g. the species Borago officinalis [borage]; Brassicaceae such as the genera Brassica, Melanosinapis, Sinapis, Arabadopsis e.g. the species Brassica napus, Brassica rapa ssp. [canola, oilseed rape, turnip rape], Sinapis arvensis Brassica juncea, Brassica juncea var. juncea, Brassica juncea var. crispifolia, Brassica juncea var. foliosa, Brassica nigra, Brassica sinapioides, Melanosinapis communis [mustard], Brassica oleracea [fodder beet] or Arabidopsis thaliana; Bromeliaceae such as the genera Anana, Bromelia e.g. the species Anana comosus, Ananas ananas or Bromelia comosa [pineapple]; Caricaceae such as the genera Carica e.g. the species Carica papaya [papaya]; Cannabaceae such as the genera Cannabis e.g. the species Cannabis sative [hemp], Convolvulaceae such as the genera Ipomea, Convolvulus e.g. the species Ipomoea batatus, Ipomoea pandurata, Convolvulus batatas, Convolvulus tiliaceus, Ipomoea fastigiata, Ipomoea tiliacea, Ipomoea triloba or Convolvulus panduratus [sweet potato, Man of the Earth, wild potato], Chenopodiaceae such as the genera Beta, i.e. the species Beta vulgaris, Beta vulgaris var. altissima, Beta vulgaris var. Vulgaris, Beta maritima, Beta vulgaris var. perennis, Beta vulgaris var. conditiva or Beta vulgaris var. esculenta [sugar beet]; Cucurbitaceae such as the genera Cucubita e.g. the species Cucurbita maxima, Cucurbita mixta, Cucurbita pepo or Cucurbita moschata [pumpkin, squash]; Elaeagnaceae such as the genera Elaeagnus e.g. the species Olea europaea [olive]; Ericaceae such as the genera Kalmia e.g. the species Kalmia latifolia, Kalmia angustifolia, Kalmia microphylla, Kalmia polifolia, Kalmia occidentalis, Cistus chamaerhodendros or Kalmia lucida [American laurel, broad-leafed laurel, calico bush, spoon wood, sheep laurel, alpine laurel, bog laurel, western bog-laurel, swamp-laurel]; Euphorbiaceae such as the genera Manihot, Janipha, Jatropha, Ricinus e.g. the species Manihot utilissima, Janipha manihot, Jatropha manihot, Manihot aipil, Manihot dulcis, Manihot manihot, Manihot melanobasis, Manihot esculenta [manihot, arrowroot, tapioca, cassava] or Ricinus communis [castor bean, Castor Oil Bush, Castor Oil Plant, Palma Christi, Wonder Tree]; Fabaceae such as the genera Pisum, Albizia, Cathormion, Feuillea, Inga, Pithecolobium, Acacia, Mimosa, Medicajo, Glycine, Dolichos, Phaseolus, Soja e.g. the species Pisum sativum, Pisum arvense, Pisum humile [pea], Albizia berteriana, Albizia julibrissin, Albizia lebbeck, Acacia berteriana, Acacia littoralis, Albizia berteriana, Albizzia berteriana, Cathormion berteriana, Feuillea berteriana, Inga fragrans, Pithecellobium berterianum, Pithecellobium fragrans, Pithecolobium berterianum, Pseudalbizzia berteriana, Acacia julibrissin, Acacia nemu, Albizia nemu, Feuilleea julibrissin, Mimosa julibrissin, Mimosa speciosa, Sericanrda julibrissin, Acacia lebbeck, Acacia macrophylla, Albizia lebbek, Feuilleea lebbeck, Mimosa lebbeck, Mimosa speciosa [bastard logwood, silk tree, East Indian Walnut], Medicago sativa, Medicago falcata, Medicago varia [alfalfa], Glycine max [soybean], Dolichos soja, Glycine gracilis, Glycine hispida, Phaseolus max or Soja hispida; Geraniaceae such as the genera Pelargonium, Cocos, Oleum e.g. the species Cocos nucifera, Pelargonium grossularioides or Oleum cocois [coconut]; Gramineae such as the genera Saccharum e.g. the species Saccharum officinarum; Juglandaceae such as the genera Juglans, Wallia e.g. the species Juglans regia, Juglans ailanthifolia, Juglans sieboldiana, Juglans cinerea, Wallia cinerea, Juglans bixbyi, Juglans californica, Juglans hindsii, Juglans intermedia, Juglans jamaicensis, Juglans major, Juglans microcarpa, Juglans nigra or Wallia nigra [walnut, black walnut, common walnut, persian walnut, white walnut, butternut, black walnut]; Lauraceae such as the genera Persea, Laurus e.g. the species laurel Laurus nobilis [bay, laurel, bay laurel, sweet bay], Persea americana Persea americana, Persea gratissima or Persea persea [avocado]; Leguminosae such as the genera Arachis e.g. the species Arachis hypogaea [peanut]; Linaceae such as the genera Linum, Adenolinum e.g. the species Linum usitatissimum, Linum humile, Linum austriacum, Linum bienne, Linum angustifolium, Linum catharticum, Linum flavum, Linum grandiflorum, Adenolinum grandiflorum, Linum lewisii, Linum narbonense, Linum perenne, Linum perenne var. lewisii, Linum pratense or Linum trigynum [flax, linseed]; Lythrarieae such as the genera Punica e.g. the species Punica granatum [pomegranate]; Malvaceae such as the genera Gossypium e.g. the species Gossypium hirsutum, Gossypium arboreum, Gossypium barbadense, Gossypium herbaceum or Gossypium thurberi [cotton]; Musaceae such as the genera Musa e.g. the species Musa nana, Musa acuminata, Musa paradisiaca, Musa spp. [banana]; Onagraceae such as the genera Camissonia, Oenothera e.g. the species Oenothera biennis or Camissonia brevipes [primrose, evening primrose]; Palmae such as the genera Elacis e.g. the species Elaeis guineensis [oil plam]; Papaveraceae such as the genera Papaver e.g. the species Papaver orientale, Papaver rhoeas, Papaver dubium [poppy, oriental poppy, corn poppy, field poppy, shirley poppies, field poppy, long-headed poppy, long-pod poppy]; Pedaliaceae such as the genera Sesamum e.g. the species Sesamum indicum [sesame]; Piperaceae such as the genera Piper, Artanthe, Peperomia, Steffensia e.g. the species Piper aduncum, Piper amalago, Piper angustifolium, Piper auritum, Piper betel, Piper cubeba, Piper longum, Piper nigrum, Piper retrofractum, Artanthe adunca, Artanthe elongata, Peperomia elongata, Piper elongatum, Steffensia elongata. [Cayenne pepper, wild pepper]; Poaceae such as the genera Hordeum, Secale, Avena, Sorghum, Andropogon, Holcus, Panicum, Oryza, Zea, Triticum e.g. the species Hordeum vulgare, Hordeum jubatum, Hordeum murinum, Hordeum secalinum, Hordeum distichon Hordeum aegiceras, Hordeum hexastichon., Hordeum hexastichum, Hordeum irregulare, Hordeum sativum, Hordeum secalinum [barley, pearl barley, foxtail barley, wall barley, meadow barley], Secale cereale [rye], Avena sativa, Avena fatua, Avena byzantina, Avena fatua var. sativa, Avena hybrida [oat], Sorghum bicolor, Sorghum halepense, Sorghum saccharatum, Sorghum vulgare, Andropogon drummondii, Holcus bicolor, Holcus sorghum, Sorghum aethiopicum, Sorghum arundinaceum, Sorghum caffrorum, Sorghum cernuum, Sorghum dochna, Sorghum drummondii, Sorghum durra, Sorghum guineense, Sorghum lanceolatum, Sorghum nervosum, Sorghum saccharatum, Sorghum subglabrescens, Sorghum verticilliflorum, Sorghum vulgare, Holcus halepensis, Sorghum miliaceum millet, Panicum militaceum [Sorghum, millet], Oryza sativa, Oryza latifolia [rice], Zea mays [corn, maize] Triticum aestivum, Triticum durum, Triticum turgidum, Triticum hybernum, Triticum macha, Triticum sativum or Triticum vulgare [wheat, bread wheat, common wheat], Proteaceae such as the genera Macadamia e.g. the species Macadamia intergrifolia [macadamia]; Rubiaceae such as the genera Coffea e.g. the species Cofea spp., Coffea arabica, Coffea canephora or Coffea liberica [coffee]; Scrophulariaceae such as the genera Verbascum e.g. the species Verbascum blattaria, Verbascum chaixii, Verbascum densiflorum, Verbascum lagurus, Verbascum longifolium, Verbascum lychnitis, Verbascum nigrum, Verbascum olympicum, Verbascum phlomoides, Verbascum phoenicum, Verbascum pulverulentum or Verbascum thapsus [mullein, white moth mullein, nettle-leaved mullein, dense-flowered mullein, silver mullein, long-leaved mullein, white mullein, dark mullein, greek mullein, orange mullein, purple mullein, hoary mullein, great mullein]; Solanaceae such as the genera Capsicum, Nicotiana, Solanum, Lycopersicon e.g. the species Capsicum annuum, Capsicum annuum var. glabriusculum, Capsicum frutescens [pepper], Capsicum annuum [paprika], Nicotiana tabacum, Nicotiana alata, Nicotiana attenuata, Nicotiana glauca, Nicotiana langsdorffii, Nicotiana obtusifolia, Nicotiana quadrivalvis, Nicotiana repanda, Nicotiana rustica, Nicotiana sylvestris [tobacco], Solanum tuberosum [potato], Solanum melongena [egg-plant] (Lycopersicon esculentum, Lycopersicon lycopersicum, Lycopersicon pyriforme, Solanum integrifolium or Solanum lycopersicum [tomato]; Sterculiaceae such as the genera Theobroma e.g. the species Theobroma cacao [cacao]; Theaceae such as the genera Camellia e.g. the species Camellia sinensis) [tea].

In another embodiment of the invention the transgenic non-human organism is a transgenic microorganism like selected from the group consisting of Charophyceae such as the genera Chara, Nitella e.g. the species Chara globularis, Chara vulgaris, Nitella flexilis, Chlorophyceae such as the genera Acrosiphonia, Spongomorpha, Urospora, Bryopsis, Pseudobryopsis, Trichosolen, Dichotomosiphon, Caulerpa, Rhipilia, Blastophysa, Avrainvillea, Chlorodesmis, Codium, Espera, Halicystis, Halimeda, Penicillus, Pseudocodium, Rhipiliopsis, Rhipocephalus, Tydemania, Udotea, Derbesia, Acrochaete, Aphanochaete, Bolbocoleon, Chaetobolus, Chaetonema, Chaetophora, Chlorotylium, Desmococcus, Draparnaldia, Draparnaldiopsis, Ectochaete, Endophyton, Entocladia, Epicladia, Internoretia, Microthamnion, Ochlochaete, Phaeophila, Pilinella, Pringsheimiella, Protoderma, Pseudendoclonium, Pseudodictyon, Pseudopringsheimia, Pseudulvella, Schizomeris, Stigeoclonium, Thamniochaete, Ulvella, Pilinia, Tellamia, Helicodictyon, Actidesmium, Ankyra, Characium, Codiolum, Sykidion, Keratococcus, Prototheca, Bracteacoccus Chlorococcum, Excentrosphaera, Hormidium, Oophila, Schroederia, Tetraedron, Trebouxia Chlorosarcinopsis, Gomphonitzschia, Coccomyxa, Dactylothece, Diógenes, Disporá, Gloeocystis, Mycanthococcus, Ourococcus, Coelastrum, Dicranochaete, Botryococcus, Dictyosphaerium, Dimorphococcus, Chlorochytrium, Kentrosphaera, Phyllobium, Gomontia, Hormotila, Euastropsis, Hydrodictyon, Pectodictyon, Pediastrum, Sorastrum, Tetrapedia, Acanthosphaera, Echinosphaerella, Echinosphaeridium, Errerella, Gloeoactinium, Golenkeniopsis Golenkinia, Micractinium, Ankistrodesmus, Chlorella, Chodatella, Closteriopsis, Cryocystis, Dactylococcus, Dematractum, Eremosphaera, Eutetramorus, Franceia, Glaucocystis, Gloeotaenium, Kirchneriella, Lagerheimiella, Monoraphidium, Nannochloris, Nephrochlamys, Nephrocytium, Oocystis, Oonephris, Pachycladon, Palmellococcus, Planktosphaeria, Polyedriopsis, Pseudoraciborskia, Quadrigula, Radiococcus, Rochiscia, Scotiella, Selanastrum, Thorakochloris, Treubaria, Trochiscia, Westella, Zoochlorella, Ostreobium, Phyllosiphon, Protosiphon, Rhodochytrium, Actinastrum, Coronastrum, Crucigenia, Dictymocystis, Enallax, Scenedesmus, Selenastrum, Tetradesmus, Tetrallantos, Tetrastrum, Chlorosarcina, Anadyomene, Valoniopsis, Ventricaria, Basicladia, Chaetomorpha, Cladophora, Lola Pithophora Rhizoclonium Chaetosphaeridium, Conochaete, Coleochaete, Oligochaetophora, Polychaetophora, Cylindrocapsa, Gongrosira, Protococcus, Acetabularia, Batophora, Bornetella, Dasycladus, Halicoryne, Neomeris, Elakatothrix, Raphidonema, Microspora, Bulbochaete, Oedocladium, Oedogonium, Prasiola, Rosenvingiella, Schizogonium, Apjohnia, Chamaedoris, Cladophoropsis, Siphonocladus, Spongocladia, Boergesenia, Boodlea, Cystodictyon, Dictyosphaeria, Ernodesmis, Microdictyon, Struvea, Valonia, Sphaeroplea, Malleochloris, Stylosphaeridium, Gloeococcus, Palmella, Palmodictyon, Palmophyllum, Pseudospherocystis, Sphaerocystis, Urococcus, Apiocystis, Chaetopeltis, Gemellicystis, Paulschulzia, Phacomyxa, Pseudotetraspora, Schizochlamys, Tetraspora, Cephaleuros, Ctenocladus, Epibolium, Leptosira, Trentepohlia, Diplochaete, Monostroma, Binuclearia, Geminella, Klebsormidium, Planetonema, Radiofilum, Stichococcus, Ulothrix, Uronema, Blidingia, Capsosiphon, Chloropelta, Enteromorpha, Percursaria, Ulva, Ulvaria, Brachiomonas, Carteria, Chlainomonas, Chlamydomonas, Chlamydonephris, Chlorangium, Chlorogonium, Cyanidium, Fortiella, Glenomonas, Gloeomonas, Hyalogonium, Lobomonas Polytoma, Pyramichlamys, Scourfieldia, Smithsonimonas, Sphaerellopsis, Sphenochloris, Spirogonium, Collodictyon, Dunaliella, Haematococcus, Stephanosphaera, Coccomonas, Dysmorphococcus, Phacotus, Pteromonas, Thoracomonas, Wislouchiella, Mascherina, Pyrobotrys, Spondylomorum, Eudorina, Gonium, Oltmannsiella, Pandorina, Platydorina, Pleodorina, Stephanoon, Volvox, Volvulina, Actinotaenium, Arthrodesmus, Bambusina Closterium, Cosmarium, Desmidium, Euastrum, Groenbladia, Hyalotheca, Micrasterias, Penium, Phymatodocis, Pleurotaenium, Sphaerozosma, Spinoclosterium, Spinocosmarium, Spondylosium, Staurastrum, Tetmemorus, Triploceras, Xanthidium, Cylindrocystis, Genicularia, Gonatozygon, Mesotaenium, Netrium, Roya, Spirotaenia, Cosmocladium, Debarya, Docidium, Euastridium, Hallasia, Mougeotia, Mougeotiopsis, Sirogonium, Spirogyra, Staurodesmus, Teilingia, Zygnema, Zygogonium, e.g. the species Caulerpa taxifolia, Prototheca wickerhamii, Ankistrodesmus falcatus, Chlorella ellipsoidea, Chlorella pyrenoidosa, Clorella sorokiniana, Chlorella vulgaris, Scenedesmus obliquus, Scenedesmus quadricauda, Selenastrum capricornutum, Selenastrum undecimnotata, Cladophora glomerata, Chlamydomonas eugametos, Chlamydomonas reinhardtii, Cyanidium caldarium, Dunaliella salina, Dunaliella tertiolecta, Euglena gracilis, Haematococcus pluvialis, Coniugatophyceae, Prasinophyceae Trebouxiophyceae, Ulvophyceae, Chlorodendraceae, Pedinomonadales, Halosphaeraceae, Pterospermataceae, Monomastigaceae, Pyramimonadaceae, Chlorodendraceae such as the genera Prasinocladus e.g. the species Prasinocladus ascus, Halosphaeraceae, Pedinomonadales, Pedinomonadaceae such as the genera Pedinomonas, Pterospermataceae such as the genera Pachysphaera, Pterosperma, Halosphaera, Pyramimonas, Bacillariophyceae, Chrysophyceae, Craspedophyceae, Euglenophyceae, Prymnesiophyceae, Phaeophyceae, Dinophyceae, Rhodophyceae, Xanthophyceae, Prasinophyceae such as the genera Nephroselmis, Prasinococcus, Scherffelia, Tetraselmis, Mantoniella, Ostreococcus e.g. the species Nephroselmis olivacea, Prasinococcus capsulatus, Scherffelia dubia, Tetraselmis chui, Tetraselmis suecica, Mantoniella squamata or Ostreococcus tauri.

Other useful organism are prokaryotic host organism, which may be useful for the cloning of the desired nucleic acid constructs or vectors such as the genera Escherichia, for example the species Escherichia coli, in particular E. coli K12 and its described strains or Agrobacterium, for example Agrobacterium tumefaciens.

The introduction of the nucleic acids according to the invention, the expression cassette or the vector into the non-human organism, like a microorganism or a plant, can in principle be done by all of the methods known to those skilled in the art. The introduction of the nucleic acid sequences gives rise to recombinant or transgenic organisms.

After the introduction of the nucleic acid, nucleic acid construct, expression cassette or vector, and the expression thereof the transgenic organism or cell is advantageously cultured and subsequently harvested.

Unless otherwise specified, the terms “polynucleotides”, “nucleic acid” and “nucleic acid molecule” as used herein are interchangeably. Unless otherwise specified, the terms “peptide”, “polypeptide” and “protein” are interchangeably in the present context. The term “sequence” may relate to polynucleotides, nucleic acids, nucleic acid molecules, peptides, polypeptides and proteins, depending on the context in which the term “sequence” is used. The terms “gene(s)”, “polynucleotide”, “nucleic acid sequence”, “nucleotide sequence”, or “nucleic acid molecule(s)” as used herein refers to a polymeric form of nucleotides of any length, either ribonucleotides or deoxyribonucleotides. The terms refer only to the primary structure of the molecule.

Thus, the terms “gene(s)”, “polynucleotide”, “nucleic acid sequence”, “nucleotide sequence”, or “nucleic acid molecule(s)” as used herein include double- and single-stranded DNA and RNA. They also include known types of modifications, for example, methylation, “caps”, substitutions of one or more of the naturally occurring nucleotides with an analog. Preferably, the DNA or RNA sequence of the invention comprises a coding sequence encoding the herein defined polypeptide.

The genes of the invention, coding for an activity selected from the group consisting of 3-ketoacyl-CoA thiolase, 3-oxoacyl-[acyl-carrier-protein] synthase, AAS bifunctional protein, ABC transporter permease protein, acid shock protein, aconitate hydratase, arginine decarboxylase, aromatic acid decarboxylase, aspartate-semialdehyde dehydrogenase, At1g19800-protein, At2g45420-protein, ATPase epsilon subunit, b0801-protein, b1330-protein, b1470-protein, b1670-protein, b1837-protein, b2032-protein, b2099-protein, b2107-protein, b2399-protein, b2474-protein, b2613-protein, b2682-protein, b2849-protein, b2909-protein, beta-hydroxylase, carbamoyl-phosphate synthase subunit, cation-transporting ATPase, CCAAT-binding transcription factor, chlorophyllase, colanic acid biosynthesis protein, coproporphyrinogen III oxidase, CTP synthetase, DnaJ-like chaperone, electron transport complex protein, ethanolamine utilization protein, fatty acid desaturase, Fe—S subunit of oxidoreductase, flavodoxin, fumarate hydratase, gamma-glutamyltranspeptidase, glucose-6-phosphate 1-dehydrogenase, glutaredoxin, glycin cleavage system protein, GM02LC38418-protein, heat shock transcription factor, hydrolase, lipoprotein precursor, membrane transport protein, metal-dependent hydrolase, molecular chaperone, monothiol glutaredoxin, multiple antibiotic resistance protein, outer membrane receptor protein precursor, oxidoreductase, phosphate import ATP-binding protein, phosphoribosylformyl glycinamidine synthase subunit, phosphotriesterase, protein kinase, protein phosphatase, pyruvate kinase, recombinase A, sll1032-protein, sn-glycerol-3-phosphate transport system permease protein, ThiF family protein, threonine dehydratase, threonine efflux protein, transcription factor, transcriptional regulator protein, tryptophan synthase alpha chain, TTC0490-protein, xanthine dehydrogenase, ydr507c-protein, and YGR104C-protein are also called “FCRP genes”.

A “coding sequence” is a nucleotide sequence, which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. The triplets taa, tga and tag represent the (usual) stop codons which are interchangeable. A coding sequence can include, but is not limited to mRNA, cDNA, recombinant nucleotide sequences or genomic DNA, while introns may be present as well under certain circumstances.

To introduce a nucleic acid molecule into a nucleic acid construct, e.g. as part of an expression cassette, the codogenic gene segment is advantageously subjected to an amplification and ligation reaction in the manner known by a skilled person. It is preferred to follow a procedure similar to the protocol for the Pfu DNA polymerase or a Pfu/Taq DNA polymerase mixture. The primers are selected according to the sequence to be amplified. The primers should expediently be chosen in such a way that the amplificate comprise the codogenic sequence from the start to the stop codon. After the amplification, the amplificate is expediently analyzed. For example, the analysis may consider quality and quantity and be carried out following separation by gel electrophoresis. Thereafter, the amplificate can be purified following a standard protocol (for example Qiagen). An aliquot of the purified amplificate is then available for the subsequent cloning step. The skilled worker generally knows suitable cloning vectors.

They include, in particular, vectors which are capable of replication in easy to handle cloning systems like as bacterial, yeast or insect cell based (e.g. baculovirus expression) systems, that is to say especially vectors which ensure efficient cloning in E. coli, and which make possible the stable transformation of plants. Vectors, which must be mentioned, in particular are various binary and cointegrated vector systems, which are suitable for the T-DNA-mediated transformation. Such vector systems are generally characterized in that they contain at least the vir genes, which are required for the Agrobacterium-mediated transformation, and the T-DNA border sequences.

In general, vector systems preferably also comprise further cisregulatory regions such as promoters and terminators and/or selection markers by means of which suitably transformed organisms can be identified. While vir genes and T-DNA sequences are located on the same vector in the case of cointegrated vector systems, binary systems are based on at least two vectors, one of which bears vir genes, but no T-DNA, while a second one bears T-DNA, but no vir gene. Owing to this fact, the last-mentioned vectors are relatively small, easy to manipulate and capable of replication in E. coli and in Agrobacterium. These binary vectors include vectors from the series pBIB-HYG, pPZP, pBecks, pGreen. Those which are preferably used in accordance with the invention are Bin19, pBI101, pBinAR, pGPTV and pCAMBIA. An overview of binary vectors and their use is given by Hellens et al, Trends in Plant Science 5, 446-451 (2000). In case of a targeted expression the vectors are preferably modified in such a manner, that they already contain the nucleic acid coding for the transit peptide and that the nuleic acids of the invention, preferentially the nucleic acid sequences encoding the polypeptides shown in the respective line in Table II, column 5 or 8, or a homolog or a fragment thereof, can be cloned 3″prime to the transit peptide encoding sequence, leading to a functional preprotein, which is directed to the intended compartment, like plastids or mitochondria, and which means that the mature protein fulfills its biological activity.

For a vector preparation, vectors may first be linearized using restriction endonuclease(s) and then be modified enzymatically in a suitable manner. Thereafter, the vector is purified, and an aliquot is employed in the cloning step. In the cloning step, the enzymecleaved and, if required, purified amplificate is cloned together with similarly prepared vector fragments, using ligase. In this context, a specific nucleic acid construct, or vector or plasmid construct, may have one or else more codogenic gene segments. The codogenic gene segments in these constructs are preferably linked operably to regulatory sequences. The regulatory sequences include, in particular, plant sequences like the above-described promoters and terminators. The constructs can advantageously be propagated stably in microorganisms, in particular Escherichia coli and/or Agrobacterium tumefaciens, under selective conditions and enable the transfer of homologous or heterologous DNA into non-human organisms, like plants or other microorganisms. In accordance with a particular embodiment, the constructs are based on binary vectors (overview of a binary vector: Hellens et al., 2000). As a rule, they contain prokaryotic regulatory sequences, such as replication origin and selection markers, for the multiplication in microorganisms such as Escherichia coli and Agrobacterium tumefaciens. Vectors can further contain agrobacterial T-DNA sequences for the transfer of DNA into plant genomes or other eukaryotic regulatory sequences for transfer into other eukaryotic cells, e.g. Saccharomyces sp. or other prokaryotic regulatory sequences for the transfer into other prokaryotic cells, e.g. Corynebacterium sp. or Bacillus sp. For the transformation of plants, the right border sequence, which comprises approximately 25 base pairs, of the total agrobacterial T-DNA sequence is advantageously included. Usually, the plant transformation vector constructs according to the invention contain T-DNA sequences both from the right and from the left border region, which contain expedient recognition sites for site-specific acting enzymes, which, in turn, are encoded by some of the vir genes.

Alternatively the nuleic acids of the invention are cloned into vectors, which are designed for the direct transformation of organelles such as for example plastids. Generally such vectors additionally carry a specific resistance gene (as mentioned above), like the spectomycin resistance gene (aad) under control of a plastid regulatory sequence and two adjacent plastome sequences of the target organism, which mediated the directed insertion of the sequences of interest, e.g. the resistance gene and the expression cassette, into the plastidal genome through homologous recombination. As transformation can be achieved by particle bombardment or other physical or chemical methods e.g. PEG treatment or microinjection, the vectors do not need to contain the elements necessary for agrobacterial T-DNA transfer (see below).

In order to introduce, into a plant, the nucleic acid molecule of the invention or used in the process according to the invention, it has proved advantageous first to transfer them into an intermediate host, for example a bacterium or a eukaryotic unicellular cell. The transformation into E. coli, which can be carried out in a manner known per se, for example by means of heat shock or electroporation, has proved itself expedient in this context. Thus, the transformed E. coli colonies can be analyzed for their cloning efficiency. This can be carried out with the aid of a PCR. Here, not only the identity, but also the integrity, of the plasmid construct can be verified with the aid of a defined colony number by subjecting an aliquot of the colonies to said PCR. As a rule, universal primers which are derived from vector sequences are used for this purpose, it being possible, for example, for a forward primer to be arranged upstream of the start ATG and a reverse primer to be arranged downstream of the stop codon of the codogenic gene segment. The amplificates are separated by electrophoresis and assessed with regard to quantity and quality.

The nucleic acid constructs, which are optionally verified, are subsequently used for the transformation of the plants or other hosts, e.g. other eukaryotic cells or other prokaryotic cells. To this end, it may first be necessary to obtain the constructs from the intermediate host. For example, the constructs may be obtained as plasmids from bacterial hosts by a method similar to conventional plasmid isolation.

The nucleic acid molecule of the invention or used in the process according to the invention can also be introduced into modified viral vectors like baculovirus vectors for expression in insect cells or plant viral vectors like tobacco mosaic virus or potato virus X-based vectors. Approaches leading to the expression of proteins from the modified viral genome including the nucleic acid molecule of the invention or used in the process according to the invention involve for example the inoculation of tobacco plants with infectious RNA transcribed in vitro from a cDNA copy of the recombinant viral genome. Another approach utilizes the transfection of whole plants from wounds inoculated with Agrobacterium tumefaciens containing cDNA copies of recombinant plus-sense RNA viruses. Different vectors and virus are known to the skilled worker for expression in different target e.g. production plants.

A large number of methods for the transformation of plants are known. Since, in accordance with the invention, a stable integration of heterologous or additional homologous DNA into the genome of plants is advantageous, the T-DNA-mediated transformation has proved expedient in particular. For this purpose, it is first necessary to transform suitable vehicles, in particular agrobacteria, with a codogenic gene segment or the corresponding plasmid construct comprising the nucleic acid molecule of the invention or an expression cassette according to the invention. This can be carried out in a manner known per se. For example, said nucleic acid construct of the invention, like said expression cassette or said plasmid construct, which has been generated in accordance with what has been detailed above, can be transformed into competent agrobacteria by means of electroporation or heat shock. In principle, one must differentiate between the formation of cointegrated vectors on the one hand and the transformation with binary vectors on the other hand. In the case of the first alternative, the constructs, which comprise the codogenic gene segment or the nucleic acid molecule of the invention have no T-DNA sequences, but the formation of the cointegrated vectors or constructs takes place in the agrobacteria by homologous recombination of the construct with T-DNA. The T-DNA is present in the agrobacteria in the form of Ti or Ri plasmids in which exogenous DNA has expediently replaced the oncogenes. If binary vectors are used, they can be transferred to agrobacteria either by bacterial conjugation or by direct transfer. These agrobacteria expediently already comprise the vector bearing the vir genes (currently referred to as helper Ti (Ri) plasmid). As mentioned before the stable integration of the heterologous (or additional homologous) nucleic acids into the plastidial genome may also be advantageously.

One or more markers may expediently also be used together with the nucleic acid construct, like the expression cassette, or the vector of the invention and, if plants or plant cells shall be transformed together with the T-DNA, with the aid of which the isolation or selection of transformed organisms, such as agrobacteria or transformed plant cells, is possible. These marker genes enable the identification of a successful transfer of the nucleic acid molecules according to the invention via a series of different principles, for example via visual identification with the aid of fluorescence, luminescence or in the wavelength range of light which is discernible for the human eye, by a resistance to herbicides or antibiotics, via what are known as nutritive markers (auxotrophism markers) or antinutritive markers, via enzyme assays or via phytohormones. Examples of such markers which may be mentioned are GFP (=green fluorescent protein); the luciferin/luceferase system, the [3-galactosidase with its colored substrates, for example X-Gal, the herbicide resistances to, for example, imidazolinone, glyphosate, phosphinothricin or sulfonylurea, the antibiotic resistances to, for example, bleomycin, hygromycin, streptomycin, kanamycin, tetracyclin, chloramphenicol, ampicillin, gentamycin, geneticin (G418), spectinomycin or blasticidin, to mention only a few, nutritive markers such as the utilization of mannose or xylose, or antinutritive markers such as the resistance to 2-deoxyglucose. This list is a small number of possible markers. The skilled worker is very familiar with such markers. Different markers are preferred, depending on the organism and the selection method. In case of plastidal transformation methods other marker genes known to a person skilled in the art may be used, but also the ones mentioned above, preferably e.g. the spectomycin resistance gene (aadA).

As a rule, it is desired that the plant nucleic acid constructs, plant expression cassettes, are flanked by T-DNA at one or both sides of the codogenic gene segment. This is particularly useful when bacteria of the species Agrobacterium tumefaciens or Agrobacterium rhizogenes are used for the transformation. A method, which is preferred in accordance with the invention, is the transformation with the aid of Agrobacterium tumefaciens. However, biolistic methods may also be used advantageously for introducing the sequences in the process according to the invention, and the introduction by means of PEG is also possible. The transformed agrobacteria can be grown in the manner known per se and are thus available for the expedient transformation of the plants. The plants or plant parts to be transformed are grown or provided in the customary manner. The transformed agrobacteria are subsequently allowed to act on the plants or plant parts until a sufficient transformation rate is reached. Allowing the agrobacteria to act on the plants or plant parts can take different forms. For example, a culture of morphogenic plant cells or tissue may be used. After the T-DNA transfer, the bacteria are, as a rule, eliminated by antibiotics, and the regeneration of plant tissue is induced. This is done in particular using suitable plant hormones in order to initially induce callus formation and then to promote shoot development.

Plant cells, plant tissues etc. may be transformed transient or stable. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least the flower primordia. The plant is subsequently grown on until the seeds of the treated plant are obtained (Clough and Bent, Plant J. 16, 735 (1998)). To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Further advantageous transformation methods, in particular for plants, are known to the skilled worker and are described herein.

Further advantageous and suitable methods are protoplast transformation by poly(ethylene glycol)-induced DNA uptake, the “biolistic” method using the gene cannon—referred to as the particle bombardment method, electroporation, the incubation of dry embryos in DNA solution, microinjection and gene transfer mediated by Agrobacterium. Said methods are described by way of example in Jenes B. et al., Techniques for Gene Transfer, in: Transgenic Plants, Vol. 1, Engineering and Utilization, eds. Kung S. D. and Wu R., Academic Press (1993) 128-143 and in Potrykus, Annu. Rev. Plant Physiol. Plant Molec. Biol. 42, 205 (1991)). The nucleic acids or the construct, e.g. the expression cassette, to be expressed is preferably cloned into a vector, which is suitable for transforming Agrobacterium tumefaciens, for example pBin19 (Bevan et al., Nucl. Acids Res. 12, 8711 (1984)). Agrobacteria transformed by an expression vector according to the invention may likewise be used in known manner for the transformation of plants such as test plants like Arabidopsis or crop plants such as cereal crops, corn, oats, rye, barley, wheat, soybean, rice, cotton, sugar beet, canola, sunflower, flax, hemp, potatoes, tobacco, tomatoes, carrots, paprika, oilseed rape, tapioca, cassava, arrowroot, tagetes, alfalfa, lettuce and the various tree, nut and vine species, in particular oil-containing crop plants such as soybean, peanut, castor oil plant, sunflower, corn, cotton, flax, oilseed rape, coconut, oil palm, safflower (Carthamus tinctorius) or cocoa bean, or in particular corn, wheat, soybean, rice, cotton and canola, e.g. by bathing bruised leaves or chopped leaves in an agrobacterial solution and then culturing them in suitable media. The transformation of plants by means of Agrobacterium tumefaciens is described, for example, by Höfgen and Willmitzer in Nucl. Acid Res. 16, 9877 (1988) or is known inter alia from White F. F., “Vectors for Gene Transfer in Higher Plants”; in Transgenic Plants, Vol. 1, Engineering and Utilization, eds. Kung S. D. and Wu R., Academic Press, 1993, pp. 15-38. For the transformation of plastids physical methods like PEG-treatment (O'Neil et al., Plant Journal. 3, 729 (1993), Golds et al., BioTechnology 11, 95 (1993)), microinjection (Knoblauch et al., Nat. Biotech. 17, 906 (1999)) or biolistics (Svab et al., Proc. Natl. Acad. Sci. USA 90, 8526 (1990)) are preferred. Such transformation methods are especially useful for the direct transformation of plastids and are well known to the skilled worker.

The expression of the nucleic acid molecules used in the process according the present invention may be desired alone or in combination with other genes or nucleic acid molecules. Multiple nucleic acid molecules conferring the expression of advantageous genes can be introduced via the simultaneous transformation of several individual suitable nucleic acid constructs, i.e. expression constructs, or, preferably, by combining several expression cassettes on one construct. It is also possible to transform the recipient non-human organisms stepwise with several vectors which in each case comprises a single expression cassette or several expression cassettes.

In addition to the sequence mentioned in Table I, application no. 8, column 5 or 8, preferably the coding region thereof, or its homologs or fragments, it may be advantageous additionally to express and/or mutate further genes in the non-human organisms. Especially advantageously, additionally at least one further gene of the biosynthetic pathway of the fine chemical is expressed in the non-human organisms such as plants or microorganisms. It is also possible that the regulation of the natural genes has been modified advantageously so that the gene and/or its gene product is no longer subject to the regulatory mechanisms which exist in the non-human organisms. This leads to an increased synthesis of the respective fatty acid since, for example, feedback regulations no longer exist to the same extent or not at all. In addition it might be advantageously to combine the nucleic acids sequences of the invention containing the sequences shown in the respective line in Table I, application no. 8, column 5 or 8, preferably the coding region thereof, or homologs or fragments thereof, with genes which generally support or enhance the growth or yield of the target non-human organisms, for example genes which lead to faster growth rate of non human organism like microorganisms or plants or genes which produce stress-, pathogen-, or herbicide-resistant plants.

In a further embodiment of the process of the invention, non-human organisms are grown, in which there is in addition to the expression of the nucleic acid molecule according to the invention simultaneous expression of at least one nucleic acid molecule or one of the genes which code for proteins involved in the fatty acid metabolism, in particular in fatty acid synthesis, especially genes coding for proteins selected from the group of acyl-CoA dehydrogenase(s), acyl-ACP [=acyl carrier protein] desaturase(s), acyl-ACP thioesterase(s), fatty acid acyltransferase(s), acyl- CoA: lysophospholipid acyltransferases, fatty acid synthase(s) , fatty acid hydroxylase(s), acetyl-coenzyme A carboxylase(s), acyl-coenzyme A oxidase(s), fatty acid desaturase(s), fatty acid acetylenases, lipoxygenases, triacylglycerol lipases, allene oxide synthases, hydroperoxide lyases or fatty acid elongase(s), Δ4-desaturases, Δ5-desaturases, Δ6-desaturases, Δ8-desaturases, Δ9-desaturases, Δ12-desaturases, Δ15-desaturases, Δ12- and Δ15-desaturases, ω-3-desaturases, Δ6-elongases, Δ9-elongases or Δ5-elongases in the cytosol or in an organelle, like plastids or mitochondria.

In a further advantageous embodiment of the process of the invention, the non-human organisms used in the process are those in which simultaneously in addition a fine chemical degrading protein is attenuated, in particular by reducing the rate of expression of the corresponding gene.

In another embodiment of the process of the invention, the non-human organisms used in the process are those in which simultaneously in addition at least one of the aforementioned nucleic acids or of the aforementioned genes is mutated in such a way that the enzymatic or biological activity of the corresponding fine chemical degrading protein is partially reduced or completely blocked. A reduction in the enzymatic or biological activity means an enzymatic or biological activity, which is reduced by at least 10%, advantageously at least 20%, 30%, 40%, 50%, 60%, 70% or more, compared with the starting organism.

If it is intended to transform the host cell, in particular the plant cell, with several constructs, expression cassettes or vectors, the marker of a preceding transformation must be removed or a further marker employed in a following transformation. The markers can be removed from the host cell, in particular the plant cell, as described herein below via methods with which the skilled worker is familiar. These methods may be not used for multiple transformed host cell only but also for single transformed host cells. In particular plants without a marker, in particular without resistance to antibiotics, are an especially preferred embodiment of the present invention.

In the process according to the invention, the nucleic acid molecules used in the process according to the invention are advantageously linked operably to one or more regulatory signals in order to increase gene expression. These regulatory sequences are intended to enable the specific expression of the genes and the expression of protein. Depending on the host organism for example plant or microorganism, this may mean, for example, that the gene is expressed and/or overexpressed after induction only, or that it is expressed and/or overexpressed constitutively. These regulatory sequences are, for example, sequences to which the inductors or repressors bind and which thus regulate the expression of the nucleic acid. In addition to these novel regulatory sequences, or instead of these sequences, the natural regulation of these sequences may still be present before the actual structural genes and, if appropriate, may have been genetically modified so that the natural regulation has been switched off and gene expression has been increased. However, the nucleic acid construct of the invention suitable as expression cassette (=expression construct=gene construct) can also be simpler in construction, that is to say no additional regulatory signals have been inserted before the nu oleic acid molecule or the homolog or fragment thereof, and the natural promoter together with its regulation has not been removed. Instead, the natural regulatory sequence has been mutated in such a way that regulation no longer takes place and/or gene expression is increased. These modified promoters can also be introduced on their own before the natural gene in the form of part sequences (=promoter with parts of the nucleic acid sequences according to the invention) in order to increase the activity. Moreover, the gene construct can advantageously also comprise one or more of what are known as enhancer sequences in operable linkage with the promoter, and these enable an increased expression of the nucleic acid sequence. Also, it is possible to insert additional advantageous sequences at the 3′ end of the DNA sequences, such as, for example, further regulatory elements or terminators. In another preferred embodiment, the natural or created expression cassette is further modified in such a manner, that a nucleic acid sequence encoding a transit peptide is functionally introduced between the regulatory and the coding region such, that a functionally preprotein is expressed, which is targeted to the organelles such as plastids or mitochondria, preferably the plastids.

The nucleic acid molecules, which encode proteins according to the invention and nucleic acid molecules, which encode other polypeptides may be present in one nucleic acid construct, expression cassette or vector or in respective several ones. Advantageously, only one copy of the nucleic acid molecule of the invention or its encoding genes is present in the nucleic acid construct, expression cassette or vector. Several vectors, nucleic acid construct, or expression cassettes can be expressed together in the host organism. The nucleic acid molecule, the nucleic acid construct or the expression cassette according to the invention can be inserted in a vector and be present in the cell in a free form. If a stable transformation is preferred, a vector is used, which is stably duplicated over several generations or which is else inserted into the genome. In the case of plants, integration into the genome of e.g. plastids or mitochondria or, in particular, into the nuclear genome may have taken place. For the insertion of more than one gene in the host genome the genes to be expressed are present together in one gene construct, for example in above-described vectors bearing a plurality of genes.

As a rule, regulatory sequences for the expression rate of a gene are located upstream (5′), within, and/or downstream (3′) relative to the coding sequence of the nucleic acid molecule of the invention or another codogenic gene segment. They control in particular transcription and/or translation and/or the transcript stability. The expression level is dependent on the conjunction of further cellular regulatory systems, such as the protein biosynthesis and degradation systems of the cell.

Regulatory sequences include transcription and translation regulating sequences or signals, e.g. sequences located upstream (5′), which concern in particular the regulation of transcription or translation initiation, such as promoters or start codons, and sequences located downstream (3′), which concern in particular the regulation of transcription or translation termination and transcript stability, such as polyadenylation signals or stop codons. Regulatory sequences can also be present in transcribed coding regions as well in transcribed non-coding regions, e.g. in introns, as for example splicing sites, promoters for the regulation of expression of the nucleic acid molecule according to the invention in a cell and which can be employed are, in principle, all those which are capable of stimulating the transcription of genes in the organisms in question, such as microorganisms or plants. Suitable promoters, which are functional in these non-human organisms are generally known. They may take the form of constitutive or inducible promoters. Suitable promoters can enable the development- and/or tissue-specific expression in multi-cell eukaryotes; thus, leaf-, root-, flower-, seed-, stomata-, tuber-, fruit- or pollen-specific promoters may advantageously be used in plants. Furthermore in case of direct transformation of organelles such as plastids, promoters recognized by the plastid RNA-polymerases such as the plastid encoded Escherichia coli-like RNA polymerase or the nuclear encoded plastid RNA polymerase may advantageously be used.

The regulatory sequences or factors can, as described above, have a positive effect on, the expression of the genes introduced, thus increasing their expression. Thus, an enhancement of the expression can advantageously take place at the transcriptional level by using strong transcription signals such as strong promoters and/or strong enhancers. In addition, enhancement of expression on the translational level is also possible, for example by introducing translation enhancer sequences, e.g., the omega-enhancer e.g. improving the ribosomal binding to the transcript, or by increasing the stability of the mRNA, e.g. by replacing the 3′UTR coding region by a region encoding a 3′UTR known as conferring an high stability of the transcript or by stabilization of the transcript through the elimination of transcript instability, so that the mRNA molecule is translated more often than the wild type. For example in plants AU-rich elements (AREs) and DST (downstream) elements destabilized transcripts. Mutagenesis studies have demonstrated that residues within two of the conserved domains, the ATAGAT and the GTA regions, are necessary for instability function. Therefore removal or mutation of such elements would obviously lead to more stable transcripts, higher transcript rates and higher protein activity. Translation enhancers are also the “overdrive sequence”, which comprises the tobacco mosaic virus 5′-untranslated leader sequence and which increases the protein/RNA ratio (Gallie et al., Nucl. Acids Research 15, 8693 (1987)).

Enhancers are generally defined as cis active elements, which can stimulate gene transcription independent of position and orientation. Different enhancers have been identified in plants, which can either stimulate transcription constitutively or tissue or stimuli specific. Well known examples for constitutive enhancers are the enhancer from the 35S promoter (Odell et al., Nature 313, 810 (1985)) or the ocs enhancer (Fromm et al., Plant Cell 1, 977 (1989)). Other examples are the G-Box motif tetramer which confers high-level constitutive expression in dicotyledonous and monocotyledonous plants (Ishige et al., Plant Journal 18, 443 (1999)) or the petE, a NT-rich sequence which act as quantitative enhancers of gene expression in transgenic tobacco and potato plants (Sandhu et al., Plant Mol Biol. 37 (5), 885 (1998)). Beside that, a large variety of cis-active elements have been described which contribute to specific expression pattern, like organ specific expression or induced expression in response to biotic or abiotic stress. Examples are elements, which provide pathogen or wound-induced expression (Rushton, Plant Cell 14, 749 (2002)) or guard cell-specific expression (Plesch, Plant Journal 28, 455 (2001)).

Advantageous regulatory sequences for the expression of the nucleic acid molecule according to the invention in microorganisms are present for example in promoters such as the cos, tac, rha, trp, tet, trp-tet, Ipp, lac, lpp-lac, laclq-, T7, T5, T3, gal, trc, ara, SP6, Λ-PR or Λ-PL promoter, which are advantageously used in Gram-negative bacteria. Further advantageous regulatory sequences are present for example in the Gram-positive promoters amy, dnaK, xylS and SPO2, in the yeast or fungal promoters ADC1, MFα, AC, P-60, UASH, MCB, PHO, CYC1, GAPDH, TEF, rp28, ADH. Promoters, which are particularly advantageous, are constitutive, tissue or compartment specific or inducible promoters. In general, “promoter” is understood as meaning, in the present context, a regulatory sequence in a nucleic acid molecule, which mediates the expression of a coding sequence segment of a nucleic acid molecule. In general, the promoter is located upstream to the coding sequence segment. Some elements, for example expression-enhancing elements such as enhancer may, however, also be located downstream or even in the transcribed region.

In principle it is possible to use all natural promoters with their regulation sequences like those named above for the expression cassette according to the invention and the method according to the invention. Also, synthetic promoters may advantageously be used, either additionally or alone, for example synthetic promoters mediating seed-specific expression such as described in, for example, WO 99/16890. In the preparation of an expression cassette various DNA fragments can be manipulated in order to obtain a nucleotide sequence, which usefully reads in the correct direction and is equipped with a correct reading frame. To connect the DNA fragments (=nucleic acids according to the invention) to one another adaptors or linkers may be attached to the fragments. The promoter and the terminator regions can usefully be provided in the transcription direction with a linker or polylinker containing one or more restriction points for the insertion of this sequence. Generally, the linker has 1 to 10, mostly 1 to 8, preferably 2 to 6, restriction points. In general the size of the linker inside the regulatory region is less than 100 bp, frequently less than 60 bp, but at least 5 bp. The promoter may be both native or homologous as well as foreign or heterologous to the host non-human organism, for example to the host plant. In the 5′-3′ transcription direction the expression cassette contains the promoter, a nucleic acid molecule which is shown in the respective line in Table I, column 5 or 8, preferably the coding region, a homolog or a fragment thereof, and a region for transcription termination. Different termination regions can be exchanged for one another in any desired fashion.

As described above, the transcription of the genes introduced should advantageously be terminated by suitable terminators at the 3′ end of the genes introduced (behind the stop codon). A terminator, which may be used for this purpose is, for example, the OCS1 terminator, the nos3 terminator or the 35S terminator. As is the case with the promoters, different terminator sequences should be used for each gene in case several genes are introduced. Terminators, which are useful in microorganisms are for example the fimA terminator, txn terminator or trp terminator. Such terminators can be rho-dependent or rho-independent.

In an embodiment of the present invention the nucleic acid construct of the present invention comprises a nucleic acid molecule as depicted in the respective line in Table I, application no. 8, column 5 or 8, preferably the coding region thereof, a fragment or a homolog thereof, and a plant promoter, such as the Big 35S promoter, the DC3 promoter, the PC Ubi promoter, the LegB4-promoter, the Super promoter, the USP promoter or the like. In another embodiment the nucleic acid construct of the present invention comprises a nucleic acid molecule as depicted in the respective line in Table I, application no. 8, column 5 or 8, preferably the coding region thereof, a fragment or a homolog thereof, and a plant promoter such as, for example, the USP, the LegB4-, the DC3 promoter or the ubiquitin promoter from parsley or other herein mentioned promoters. In a further embodiment the nucleic acid construct of the present invention comprises a nucleic acid molecule as depicted in the respective line in Table I, application no. 8, column 5 or 8, preferably the coding region thereof, a fragment or a homolog thereof, and a plant promoter, such as the Big 35S promoter, the PC Ubi promoter, the

Super promoter, the USP promoter or the like. In addition different terminators may advantageously be used in these nucleic acid constructs; examples for transcriptional termination are polyadenylation signals.

In order to ensure the stable integration, into the transgenic plant, of nucleic acid molecules used in the process according to the invention in combination with further genes, especially biosynthesis genes, over a plurality of generations, it may be advantageous to express each of the respective coding regions used in the process under the control of its own, preferably unique, promoter.

The nucleic acid construct is advantageously constructed in such a way that a promoter is followed by a suitable cleavage site for insertion of the nucleic acid to be expressed, advantageously in a polylinker, followed, if appropriate, by a terminator located behind the polylinker. If appropriate, this order is repeated several times so that several genes are combined in one construct and thus can be introduced into the transgenic plant in order to be expressed.

The sequence is advantageously repeated up to three times. For the expression, the nucleic acid sequences are inserted via the above-mentioned suitable cleavage site, for example in the polylinker behind the promoter. It is advantageous for each nucleic acid sequence to have its own promoter and, if appropriate, its own terminator, as mentioned above. However, it is also possible to insert several nucleic acid sequences behind a promoter and, if appropriate, before a terminator if a polycistronic transcription is possible in the host or target cells. In this context, the insertion site, or the sequence of the nucleic acid molecules inserted, in the nucleic acid construct is not decisive, that is to say a nucleic acid molecule can be inserted in the first or last position in the cassette without this having a substantial effect on the expression. However, it is also possible to use only one promoter type in the construct.

Accordingly, in a preferred embodiment, the nucleic acid construct according to the invention confers expression of the nucleic acid molecule of the invention, and, optionally further genes, in a plant and comprises one or more plant regulatory elements. Said nucleic acid construct according to the invention advantageously encompasses beneath the nucleic acid molecule of the invention a plant promoter or a plant terminator or a plant promoter and a plant terminator. In another embodiment said nucleic acid construct according to the invention advantageously encompasses beneath the nucleic acid molecule of the invention a microorganism promoter or a microorganism terminator or a microorganism promoter and a microorganism terminator.

A “plant” promoter comprises regulatory elements, which mediate the expression of a coding sequence segment in plant cells. The plant promoter can originate from a plant cell, e.g. from the plant, which is transformed with the nucleic acid construct, expression cassette, or vector as described herein. However, a plant promoter does not need to be of plant origin, but may originate from viruses or microorganisms, in particular for example from viruses which attack plant cells. This also applies to other plant regulatory signals, for example in plant terminators. The term plant promoter shall also encompass organelle, especially plastidal promoters.

A nucleic acid construct suitable for plant expression preferably comprises regulatory elements which are capable of controlling the expression of genes in plant cells and which are operably linked so that each sequence can fulfill its function. Accordingly, the nucleic acid construct can also comprise transcription terminators. Examples for transcriptional termination are polyadenylation signals. Preferred polyadenylation signals are those which originate from Agrobacterium tumefaciens T-DNA, such as the gene 3 of the Ti plasmid pTiACH5, which is known as octopine synthase (Gielen et al., EMBO J. 3, 835 (1984)) or functional equivalents thereof, but all the other terminators which are functionally active in plants are also suitable.

Other preferred sequences for use in operable linkage in gene expression constructs are targeting sequences, which are required for targeting the gene product into specific cell compartments (for a review, see Kermode, Crit. Rev. Plant Sci. 15 (4), 285 (1996) and references cited therein), for example into the vacuole, the nucleus, all types of plastids, such as amyloplasts, chloroplasts, chromoplasts, the extracellular space, the mitochondria, the endoplasmic reticulum, elaioplasts, peroxisomes, glycosomes, and other compartments of cells or extracellular; preferred are sequences, which are involved in targeting to plastids as mentioned above. Sequences, which must be mentioned in this context are, in particular, the signal-peptide- or transit-peptide-encoding sequences which are known per se. For example, plastid-transit-peptide-encoding sequences enable the targeting of the expression product into the plastids of a plant cell. Targeting sequences are also known for eukaryotic and to a lower extent for prokaryotic organisms and can advantageously be operable linked with the nucleic acid molecule of the present invention as shown in Table I, application no. 8, columns 5 and 8, preferably the coding region thereof, or homologs or fragments thereof, and described herein to achieve an expression in one of said compartments or extracellular.

For expression in plants, the nucleic acid molecule must, as described above, be linked operably to or comprise a suitable promoter which expresses the gene at the right point in time and in a cell- or tissue-specific manner. Usable promoters are constitutive promoters (Benfey et al., EMBO J. 8, 2195 (1989)), such as those which originate from plant viruses, such as 35S CAMV (Franck et al., Cell 21, 285 (1980)), 19S CaMV (see also U.S. Pat. No. 5,352,605 and WO 84/02913), 34S FMV (Sanger et al., Plant. Mol. Biol., 14, 433 (1990)), the parsley ubiquitin promoter, or plant promoters such as the Rubisco small subunit promoter described in U.S. Pat. No. 4,962,028 or the plant promoters PRP1 (Ward et al., Plant. Mol. Biol. 22 (1993)), SSU, PGEL1, OCS (Leisner, Proc. Natl. Acad. Sci. USA 85 (5), 2553 (1988)), lib4, usp, mas (Comai, Plant Mol. Biol. 15 (3), 373 (1990)), STLS1, ScBV (Schenk, Plant Mol. Biol. 39 (6),1221 (1999)), B33, SAD1 or SAD2 (flax promoters, Jain et al., Crop Science, 39 (6), 1696 (1999)) or nos (Shaw et al., Nucleic Acids Res. 12 (20), 7831 (1984)). Stable, constitutive expression of the proteins according to the invention into a plant can be advantageous. However, inducible expression of the polypeptide of the invention may be advantageous, if, for example, a late expression before the harvest is of advantage.

The expression of plant genes can also be facilitated as described above via a chemical inducible promoter (for a review, see Gatz, Annu. Rev. Plant Physiol. Plant Mol. Biol. 48, 89 (1997)). Chemically inducible promoters are particularly suitable when it is desired to express the gene in a time-specific manner. Examples of such promoters are a salicylic acid inducible promoter (WO 95/19443), a benzenesulfonamide inducible promoter (EP 388 186), and abscisic acid-inducible promoter (EP 335 528), a tetracyclin-inducible promoter (Gatz et al., Plant J. 2, 397 (1992)), a cyclohexanol- or ethanol-inducible promoter (WO 93/21334) or others as described herein.

Other suitable promoters are those which react to biotic or abiotic stress conditions, for example the pathogen-induced PRP1 gene promoter (Ward et al., Plant. Mol. Biol. 22, 361 (1993)), the auxin-induced GH3-promotor (Liu et al., Plant Cell 6, 645 (1994)), the cold/dehydration/ABA-induced Cor15A-promoter (Baker et al., Plant Mol. Biol. 24, 701 (1994)), the cold/drought/salt/ABA/wound-induced Cor78 promoter (Ishitani et al., Plant Cell 9, 1935 (1997), Yamaguchi-Shinozaki and Shinozaki, Plant Cell 6, 251 (1994)), the cold/dehydration-induced Rci2A-promoter (Capel et al., Plant Physiol. 115, 569 (1997)), the drought/salt-induced Rd22-promoter (Yamaguchi-Shinozaki and Shinozaki, Mol. Gen. Genet. 238, 17 (1993)), the salt-induced RD29A-promoter (Yamaguchi-Shinozalei et al., Mol. Gen. Genet. 236, 331 (1993)), the salt-induced ARSK1-root promoter (Hwang and Goodman, Plant J. 8, 37 (1995), the salt-induced PtxA-root promoter (GenBank accession X67427), the tomato heat-inducible hsp80 promoter (U.S. Pat. No. 5,187,267), the potato chill-inducible alpha-amylase promoter (WO 96/12814) or the wound-inducible pinl I promoter (EP-A-0 375 091) or others as described herein.

Preferred promoters are in particular those which bring about gene expression in tissues and organs, such as leaves, roots, seeds, pollen or xylem. Examples of tissue preferred or organ preferred promoters include, but are not limited to fruit-preferred, ovulepreferred, male tissue-preferred, seed-preferred, pollen-preferred integument-preferred, tuberpreferred, stalk-preferred, pericarp-preferred, leaf-preferred, stigma-preferred, pollen-preferred, anther-preferred, petal-preferred, sepal-preferred, pedicel-preferred, silique-preferred, stem-preferred, root-preferred promoters, and the like. Especially those promoters are preferred which bring about gene expression in tissues and organs in which the biosynthesis of fatty acids takes place, like in seed cells, such as endosperm cells and cells of the developing embryo. Seed promoters are preferentially expressed during seed development and/or germination. For example, seed preferred promoters can be embryo-preferred, endosperm preferred and seed coat-preferred (see Thompson et al., BioEssays 10, 108 (1989)). Examples of seed preferred promoters include, but are not limited to, cellulose synthase (celA), Cim1, gamma-zein, globulin 1, maize 19 kD zein (cZ19B1), and the like. Other suitable promoters are the oilseed rape napin gene promoter (U.S. Pat. No. 5,608,152), the Vicia faba USP promoter (Baeumlein et al., Mol Gen Genet, 225 (3), 459 (1991)), the Arabidopsis oleosin promoter (WO 98/45461), the Phaseolus vulgaris phaseolin promoter (U.S. Pat. No. 5,504,200), the Brassica Bce4 promoter (WO 91/13980), the bean arc5 promoter, the carrot DcG3 promoter, or the Legumin B4 promoter (LeB4) (Baeumlein et al., Plant Journal, 2 (2), 233 (1992)), and promoters which bring about the seed-specific expression in monocotyledonous plants such as maize, barley, wheat, rye, rice and the like. Advantageous seed-specific promoters are the sucrose binding protein promoter (WO 00/26388), the phaseolin promoter and the napin promoter. Suitable promoters which must be considered are the barley lpt2 or lpt1 gene promoter (WO 95/15389 and WO 95/23230), and the promoters described in WO 99/16890 (promoters from the barley hordein gene, the rice glutelin gene, the rice oryzin gene, the rice prolamin gene, the wheat gliadin gene, the wheat glutelin gene, the maize zein gene, the oat glutelin gene, the sorghum kasirin gene and the rye secalin gene). Further suitable promoters are Amy32b, Amy 6-6 and Aleurain (U.S. Pat. No. 5,677,474), Bce4 (oilseed rape) (U.S. Pat. No. 5,530,149), glycinin (soya) (EP 571 741], phosphoenolpyruvate carboxylase (soya) (JP 06/62870), ADR12-2 (soya) (WO 98/08962), isocitrate lyase (oilseed rape) (U.S. Pat. No. 5,689,040]) or a-amylase (barley) (EP 781 8499. Other promoters which are available for the expression of genes in plants are leaf-specific promoters such as those described in DE-A 19 644 478 or lightregulated promoters such as, for example, the pea petE promoter.

Further suitable plant promoters are the cytosolic FBPase promoter or the potato ST-LSI promoter (Stockhaus et al., EMBO J. 8, 2445 (1989)), the Glycine max phosphoribosylpyrophosphate amidotransferase promoter (GenBank Accession No. U87999) or the noduline-specific promoter described in EP 249 676.

Promoters, which are particularly suitable, are those which bring about plastid-specific expression. Suitable promoters such as the viral RNA polymerase promoter are described in WO 95/16783 and WO 97/06250, and the Arabidopsis clpP promoter, which is described in WO 99/46394.

Promoters, which are used for the strong expression of heterologous sequences as well as additional homologous sequences in as many tissues as possible, in particular also in leaves, are, in addition to several of the above-mentioned viral and bacterial promoters, preferably, plant promoters of actin or ubiquitin genes such as, for example, the rice actin1 promoter. Further examples of constitutive plant promoters are the sugarbeet V-ATPase promoters (WO 01/14572). Examples of synthetic constitutive promoters are the Super promoter (WO 95/14098) and promoters derived from G-boxes (WO 94/12015). If appropriate, chemical inducible promoters may furthermore also be used, as described in EP 388 186, EP 335 528, WO 97/06268.

Promoters, which are particularly suitable, are e.g. the super-promoter (Ni et al., Plant Journal 7, 661 (1995)), the ubiquitin promoter (Callis et al., J. Biol. Chem., 265, 12486 (1990); U.S. Pat. No. 5,510,474; U.S. Pat. No. 6,020,190; Kawalleck et al., Plant. Molecular Biology, 21, 673 (1993)) or the 34S promoter (GenBank Accession numbers M59930 and X16673). Promoters useful in the expression cassettes of the invention include, but are not limited to, the major chlorophyll a/b binding protein promoter, histone promoters, the Ap3 promoter, the 6-conglycin promoter, the napin promoter, the soybean lectin promoter, the maize 15kD zein promoter, the 22kD zein promoter, the 27kD zein promoter, the g-zein promoter, the waxy, shrunken 1, shrunken 2 and bronze promoters, the Zm13 promoter (U.S. Pat. No. 5,086,169), the maize polygalacturonase promoters (PG) (U.S. Pat. Nos. 5,412,085 and 5,545,546), and the SGB6 promoter (U.S. Pat. No. 5,470,359), the rice cyclophilin promoter (Buchholz et al., Plant Mol. Biol.25 (5), 837 (1994)), the glutelin (rice) promoter (Takaiwa et al., Mol. Gen. Genet. 208, 15 (1986), Takaiwa et al., FEBS letts. 221, 43 (1987), the rice prolamin NRP33 promoter and the rice aglobulin Glb-1 promoter (Wu et al., Plant cell Physiology 39 (8), 885 (1998),as well as synthetic or other natural promoters.

As already mentioned herein, further regulatory sequences, which may be expedient, if appropriate, also include sequences, which target the transport and/or the localization of the expression products. Sequences, which must be mentioned in this context are, in particular, the signal-peptide- or transit-peptide-encoding sequences which are known per se.

For example, plastid-transit-peptide-encoding sequences enable the targeting of the expression product into the plastids of a plant cell.

One embodiment of the present invention also relates to a method for generating a vector, which comprises the insertion of the nucleic acid molecule according to the invention or the expression cassette according to the invention into a nucleic acid molecule to give the vector. The vector can, for example, be introduced in to a cell, e.g. a microorganism or a plant cell, a plant or a part therof, as described herein for the nucleic acid construct, or below under transformation or transfection or shown in the examples. A transient or stable transformation of the host or target cell is possible, however, a stable transformation is preferred. The vector according to the invention is preferably a vector, which is suitable for expressing the polypeptide according to the invention in a microorganism or a plant cell, a plant or a part thereof. The method can thus also encompass one or more steps for integrating regulatory signals into the vector, in particular signals, which mediate the expression in microorganisms or plant cells, plants or respective parts thereof.

Accordingly, the present invention also relates to a vector comprising the nucleic acid molecule characterized herein as part of a nucleic acid construct suitable for expression in plants and/or microorganisms or the nucleic acid molecule according to the invention.

The advantageous vectors of the invention comprise the nucleic acid molecules which encode proteins according to the invention, nucleic acid molecules which are used in the process, or nucleic acid construct suitable for plant expression or for microorganism expression comprising the nucleic acid molecules used, or expression cassettes according to the invention, either alone or in combination with further genes such as the biosynthesis or regulatory genes of the fine chemical metabolism e.g. with the genes mentioned herein above.

The recombinant expression vectors which are advantageously used in the process comprise the nucleic acid molecules according to the invention or the nucleic acid construct according to the invention, or the expression cassettes according to the invention, in a form which is suitable for expressing, in a host cell, the nucleic acid molecules according to the invention or described herein. Accordingly, the recombinant expression vectors in addition may comprise one or more regulatory signals selected on the basis of the host cells to be used for the expression, in operable linkage with the nucleic acid sequence to be expressed. Furthermore, if desired, in addition the vector can comprise plastome sequences of the recipient organism to facilitate integration into the plastidal genome by homologous recombination as mentioned above.

The recombinant expression vectors used can be designed specifically for the expression, in prokaryotic and/or eukaryotic cells, of nucleic acid molecules used in the process. This is advantageous since intermediate steps of the vector construction are frequently carried out in microorganisms for the sake of simplicity. For example, the genes according to the invention and other genes can be expressed in bacterial cells, insect cells (using baculovirus expression vectors), yeast cells and other fungal cells (Romanos, Yeast 8, 423 (1992); van den Hondel, in “More Gene Manipulations in Fungi”, ed. Bennet J. W., Lasure L. L., pp. 396-428, Academic Press, San Diego (1991); van den Hondel C. A. M. J. J., in “Applied Molecular Genetics of Fungi”, ed. Peberdy J. F. et al., pp. 1-28, Cambridge University Press, Cambridge(1991)), algae (Falciatore et al., Marine Biotechnology.1 (3), 239 (1999)) using vectors and following a transformation method as described in WO 98/01572, and preferably in cells of multi-celled plants (see Schmidt R. and Willmitzer L., Plant Cell Rep. 7, 583 (1988); “Plant Molecular Biology and Biotechnology”, C Press, Boca Raton, Fla., chapter 6/7, pp.71-119 (1993); White F. F., in “Transgenic Plants”, Bd. 1, Engineering and Utilization, ed. Kung and Wu R., Academic Press, 128-43 (1993); Potrykus, Annu. Rev. Plant Physiol. Plant Molec. Biol. 42, 205 (1991) (and references cited therein)). Suitable host cells are furthermore discussed in Goeddel, “Gene Expression Technology: Methods in Enzymology 185”, Academic Press, San Diego, Calif. (1990). As an alternative, the sequence of the recombinant expression vector can be transcribed and translated in vitro, for example using T7 promoter-regulatory sequences and T7 polymerase.

In the event it is necessary proteins can be expressed in prokaryotes using vectors comprising constitutive or inducible promoters, which control the expression of fusion proteins or nonfusion proteins as mentioned above.

Other vectors which are suitable in prokaryotic organisms are known to the skilled worker; these vectors are for example in E. coli pLG338, pACYC184, the pBR series, such as pBR322, the pUC series such as pUC18 or pUC19, the M113mp series, pKC30, pRep4, pHS1, pHS2, pPLc236, pMBL24, μLG200, pUR290, pIN-III-B1, lambda-gt11 or pBdCI, in Streptomyces pIJ101, pIJ364, pIJ702 or pIJ361, in Bacillus pUB110, pC194 or pBD214, in Corynebacterium pSA77 or pAJ667.

In a further embodiment, the expression vector is a yeast expression vector. Examples of vectors for expression in the yeasts S. cerevisiae encompass pYeDesaturasec1 (Baldari et al., Embo J. 6, 229 (1987)), pMFa (Kurjan and Herskowitz, Cell 30, 933 (1982)), pJRY88 (Schultz et al., Gene 54, 113(1987)) and pYES2 (Invitrogen Corporation, San Diego, Calif.). Vectors and methods for the construction of vectors which are suitable for use in other fungi, such as the filamentous fungi, encompass those which are described in detail in: van den Hondel C. A. M. J. J.in “Applied Molecular Genetics of Fungi”, Peberdy J. F., ed., pp. 1-28, Cambridge University Press, Cambridge (1991); or van den Hondel C. A. M. J. J. in “More Gene Manipulations in Fungi”, Bennet J. W. & Lasure L. L., ed., pp. 396-428, Academic Press, San Diego (1991). Examples of other suitable yeast vectors are 2alphaM, pAG-1, YEp6, YEp13 or pEMBLYe 23.

Further vectors, which may be mentioned by way of example, are pALS1, pl L2 or pBB116 in fungi or pLGV23, pGHlac+, pBIN19, pAK2004 or pDH51 in plants.

As an alternative, the nucleic acid sequences can be expressed in insect cells using baculovirus expression vectors. Baculovirus vectors, which are available for expressing proteins in cultured insect cells (for example Sf9 cells) encompass the pAc series (Smith et al., Mol. Cell Biol. 3, 2156 (1983)) and the pVL series (Lucklow and Summers, Virology 170, 31 (1989)).

The above-mentioned vectors are only a small overview of potentially suitable vectors. Further plasmids are known to the skilled worker and are described, for example, in “Cloning Vectors” (ed. Pouwels P. H., et al., Elsevier, Amsterdam-New York-Oxford, 1985, ISBN 0 444 904018). Further suitable expression systems for prokaryotic and eukaryotic cells, see the chapters 16 and 17 by Sambrook J., Fritsch E.F. and Maniatis T., “Molecular Cloning: A Laboratory Manual”, 2nd edition, Cold Spring Harbor Laboratory, Cold Spring Harbor Laboratory Press, Cold Spring Harbor, N.Y., 1989.

An embodiment of the invention relates to a vector where the nucleic acid molecule according to the invention is linked operably to regulatory sequences which permit the expression in a prokaryotic or eukaryotic host. A further embodiment of the invention relates to a vector in which a nucleic acid sequence encoding one of the polypeptides shown in the respective line in Table II, application no. 8, columns 5 or 8, or homologs or fragments thereof, is functionally linked to a targeting sequence such as a plastidial or mitochondrial targeting sequence. A further embodiment of the invention relates to a vector in which a nucleic acid sequence encoding one of the polypeptides shown in the respective line in Table II, application no. 8, columns 5 or 8, or homologs or fragments thereof, is functionally linked to (a) regulatory sequence which permit the expression in an organelle, such as plastids or mitochondria, preferably plastids.

A further embodiment of the invention relates to a vector which comprises a nucleic acid sequence encoding one of the polypeptides shown in the respective line in Table II, application no. 8, columns 5 or 8, or homologs or fragments thereof, but no additional targeting sequence.

Accordingly, one embodiment of the invention relates to a host cell, which has been transformed stably or transiently with the vector according to the invention or the nucleic acid molecule according to the invention or the nucleic acid construct according to the invention or the expression construct according to the invention.

The genetically modified cells may be regenerated to the respective non-human organism, for example a plant cell to a plant, by all of the methods known to those skilled in the art. Appropriate methods can be found in the publications referred to above by Kung S. D. and Wu R., Potrykus or Hofgen and Willmitzer.

Accordingly, a further aspect of the invention relates to transgenic non-human organisms transformed by at least one nucleic acid molecule, expression cassette or vector according to the invention as well as cells, cell cultures, tissue, parts—such as, for example, leaves, roots, pollen, etc. in the case of plant organisms—or reproductive material derived from such non-human organisms. The terms “host organism”, “host cell”, “recombinant (host) non-human organism” and “transgenic (host) cell” are used here interchangeably. Of course these terms relate not only to the particular host non-human organism or the particular target cell but also to the descendants or potential descendants of these non-human organisms or cells. Since, due to mutation or environmental effects certain modifications may arise in successive generations, these descendants need not necessarily be identical with the parental cell but nevertheless are still encompassed by the term as used here.

For the purposes of the invention “transgenic” or “recombinant” means with regard for example to a nucleic acid molecule, a nucleic acid construct, an expression cassette or a vector containing the nucleic acid molecule according to the invention or an non-human organism transformed with/by the nucleic acid molecule, nucleic acid construct, expression cassette or vector according to the invention all those constructions produced by genetic engineering methods in which either
(a) the nucleic acid molecule depicted in the respective line in Table I, column 5 or 8, preferably the coding region thereof, or homologs or fragments thereof; or
(b) a genetic control sequence functionally linked to the nucleic acid molecule described under (a), for example a 3′- and/or 5′- genetic control sequence such as a promoter or terminator, or
(c)
(a) and (b);
   are not found in their natural, genetic environment or have been modified by genetic engineering methods, wherein the modification may by way of example be a substitution, addition, deletion, inversion or insertion of one or more nucleotide residues. Natural genetic environment means the natural genomic or chromosomal locus in the organism of origin or inside the host non-human organism or presence in a genomic library. In the case of a genomic library the natural genetic environment of the nucleic acid sequence is preferably retained at least in part. The environment sequence borders the nucleic acid sequence at least on one side and has a sequence length of at least 50 bp, preferably at least 500 bp, particularly preferably at least 1,000 bp, most particularly preferably at least 5,000 bp. A naturally occurring expression cassette—for example the naturally occurring combination of the natural promoter of the nucleic acid sequence according to the invention with the corresponding gene—turns into a transgenic expression cassette when the latter is modified by unnatural, synthetic (“artificial”) methods such as by way of example a mutagenesis. In case a non-human organism is transformed by a homologous nucleic acid molecule or by a homologous expression cassette the resulting non-human organism represents also a respective transgenic non-human organism since the additional homologous nucleic acid molecule or the additional homologous expression cassette is not located in its original environment. Appropriate methods are described by way of example in U.S. Pat. No. 5,565,350 or WO 00/15815. In another embodiment the non-human organism comprises a heterologous nucleic acid molecule.

Suitable organisms or host organisms for the nucleic acid molecule, nucleic acid construct, expression cassette or vector according to the invention are advantageously in principle all non-human organisms, which are suitable for the expression of recombinant genes as described above. Preferred non-human organism are microorganism or plants as described above, in particular those plants, which can be transformed in a suitable manner. These include monocotyledonous and dicotyledonous plants. In an embodiment of the present invention plants like Arabidopsis, Asteraceae such as Calendula or crop plants such as soybean, peanut, castor oil plant, sunflower, flax, corn, cotton, oilseed rape, coconut, oil palm, safflower (Carthamus tinctorius) or cocoa bean are preferred. In another embodiment agriculturally useful plants such as cereals and grasses, for example Triticum spp., Zea mays, Hordeum vulgare, oats, Secale cereale, Oryza sativa, Pennisetum glaucum, Sorghum bicolor, Triticale, Agrostis spp., Cenchrus ciliaris, Dactylis glomerata, Festuca arundinacea, Lolium spp., Medicago spp. and Saccharum spp., legumes and oil crops, for example Brassica juncea, Brassica napus, Glycine max, Arachis hypogaea, Gossypium hirsutum, Cicer arietinum, Helianthus annuus, Lens culinaris, Linum usitatissimum, Sinapis alba, Trifolium repens and Vicia narbonensis, vegetables and fruits, for example bananas, grapes, Lycopersicon esculentum, asparagus, cabbage, watermelons, kiwi fruit, Solanum tuberosum, Beta vulgaris, cassava and chicory, trees, for example Coffea species, Citrus spp., Eucalyptus spp., Picea spp., Pinus spp. and Populus spp., medicinal plants and trees, and flowers are preferred.

In another embodiment of the invention host plants for the nucleic acid molecule, nucleic acid construct, expression cassette or vector according to the invention are selected from the group comprising corn, soy, oil seed rape (including canola and winter oil seed rape), cotton, wheat and rice.

A further object of the invention relates to the use of a nucleic acid construct, e.g. an expression cassette, comprising one or more nucleic acid molecules encoding one or more polypeptides as depicted in the respective line(s) in Table II, application no. 8, columns 5 or 8, or homologs or fragments thereof, or comprising one or more nucleic acid molecules as depicted in the respective line(s) in Table I, application no. 8, columns 5 or 8, preferably the coding region thereof, or homologs or fragments thereof, or comprising a nucleic acid molecule hybridizing therewith for the transformation of a microorganism or a plant cell, plant tissues or a part of a plant.

In doing so, depending on the choice of promoter, the nucleic acid molecules shown in the respective line in Table I, application no. 8, column 5 or 8, preferably the coding region thereof, or homologs or fragments thereof, or the nucleic acid molecules encoding a polypeptide as depicted in the respective line in Table II, application no. 8, column 5 or 8, or homologs or fragments thereof, can be expressed in the whole plant or specifically e.g. in the leaves, in the seeds, the nodules, in roots, in the stem or other parts of the plant. Those transgenic plants overproducing sequences, e.g. as depicted in the respective line in Table I, application no. 8, column 5 or 8, preferably the coding region thereof, or homologs or fragments thereof, the plant cell, plant tissues, plants, the reproductive material thereof, or parts thereof are a further object of the present invention.

Within the framework of the present invention, the production of the linoleic acid is due to the generation or over-expression of one or more polypeptides as depicted in the respective line(s) in Table II, application no. 8, column 5 or 8, or homologs or fragments thereof, or encoded by the corresponding nucleic acid molecules as depicted in the respective line(s) in Table I, application no. 8, column 5 or 8, preferably the coding region thereof, or homologs or fragments thereof, in the non-human organism according to the invention, advantageously in the transgenic microorganism or plant according to the invention, at least for the duration of at least one plant generation.

A constitutive expression of the polypeptide as depicted in the respective line in Table II, application no. 8, column 5 or 8, or homologs or fragments thereof, or encoded by the respective nucleic acid molecule as depicted in the respective line in Table I, application no. 8, column 5 or 8, preferably the coding region thereof, or homologs or fragments thereof, may be advantageous. On the other hand, an inducible expression may also appear desirable. Expression of the polypeptide sequences of the invention can be either directed to the cytosol or to the organelles, such as plastids or mitochondria, preferably the plastids of the host cells, preferably the plant cells, or non-targeted.

The efficiency of the expression of the polypeptides depicted in the respective line in Table II, column 5 or 8, or homologs or fragments thereof, or encoded by the respective nucleic acid molecule as depicted in the respective line in Table I, column 5 or 8, preferably the coding region thereof, or homologs or fragments thereof, can be determined, for example, on test plants in greenhouse trials.

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 8, column 5 or 8, preferably shown in Table II A, application no. 8, column 5, or in Table II A, application no. 8, column 8, or in Table II B, application no. 8, column 8, or a homolog or a fragment thereof;
   (b) a nucleic acid molecule shown in Table I, application no. 8, column 5 or 8, preferably shown in Table I A, application no. 8, column 5, or in Table I A, application no. 8, column 8, or in Table I B, application no. 8, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 8, preferably shown in Table II A, application no. 8, column 5, or in Table II A, application no. 8, column 8, or in Table II B, application no. 8, column 8;
   (d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 8, preferably shown in Table I A, application no. 8, column 5, or in Table I A, application no. 8, column 8, or in Table I B, application no. 8, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 8;
   (i) a nucleic acid molecule encoding a polypeptide having the activity represented by a respective protein as depicted in column 5 of Table II, application no. 8;
   (j) a nucleic acid molecule comprising a polynucleotide which is obtained by amplifying a cDNA library or a genomic library using the primers in Table III, application no. 8, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising an expression cassette comprising
[0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 8, column 5 or 8, preferably shown in Table II A, application no. 8, column 5, or in Table II A, application no. 8, column 8, or in Table II B, application no. 8, column 8, or a homolog or a fragment thereof;
   (b) a nucleic acid molecule shown in Table I, application no. 8, column 5 or 8, preferably shown in Table I A, application no. 8, column 5, or in Table I A, application no. 8, column 8, or in Table I B, application no. 8, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 8, preferably shown in Table II A, application no. 8, column 5, or in Table II A, application no. 8, column 8, or in Table II B, application no. 8, column 8;
   (d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 8, preferably shown in Table I A, application no. 8, column 5, or in Table I A, application no. 8, column 8, or in Table I B, application no. 8, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99,5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 8;
   (i) a nucleic acid molecule encoding a polypeptide having the activity represented by a respective protein as depicted in column 5 of Table II, application no. 8;
   (j) a nucleic acid molecule comprising a polynucleotide which is obtained by amplifying a cDNA library or a genomic library using the primers in Table III, application no. 8, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising or being transformed by a vector comprising
[1) a nucleic acid molecule selected from the group consisting of
   (a) a nucleic acid molecule encoding a polypeptide shown in Table II, application no. 8, column 5 or 8, preferably shown in Table II A, application no. 8, column 5, or in Table II A, application no. 8, column 8, or in Table II B, application no. 8, column 8, or a homolog or a fragment thereof;
   (b) a nucleic acid molecule shown in Table I, application no. 8, column 5 or 8, preferably shown in Table I A, application no. 8, column 5, or in Table I A, application no. 8, column 8, or in Table I B, application no. 8, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 8, preferably shown in Table II A, application no. 8, column 5, or in Table II A, application no. 8, column 8, or in Table II B, application no. 8, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 8, preferably shown in Table I A, application no. 8, column 5, or in Table I A, application no. 8, column 8, or in Table I B, application no. 8, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 8;
   (i) a nucleic acid molecule encoding a polypeptide having the activity represented by a respective protein as depicted in column 5 of Table II, application no. 8;
   (j) a nucleic acid molecule comprising a polynucleotide which is obtained by amplifying a cDNA library or a genomic library using the primers in Table III, application no. 8, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 8.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 8 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 8, in the same line as of the respective nucleic acid molecule according to 1) “plastidic” is depicted, or encoding a mitochondrial transit peptide, especially in case in column 6 of Table I, application no. 8, in the same line as of the respective nucleic acid molecule according to 1) “mitochondrial” is depicted.

In another embodiment of said transgenic non-human organism the expression cassette does not comprise 2) a nucleic acid molecule encoding a transit peptide, especially in case in column 6 of Table I, application no. 8, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 8.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 8, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 8, in the same line as of the respective nucleic acid molecule according to 1) “plastidic” is depicted, or encoding a mitochondrial transit peptide, especially in case in column 6 of Table I, application no. 8, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 8, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 8, in the same line as of the respective nucleic acid molecule according to 1) “plastidic” is depicted, or encoding a mitochondrial transit peptide, especially in case in column 6 of Table I, application no. 8, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 8, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 8, in the same line as of the respective nucleic acid molecule according to 1) “plastidic” is depicted, or encoding a mitochondrial transit peptide, especially in case in column 6 of Table I, application no. 8, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 8.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 8, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 8.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 8, in the same line as of the respective nucleic acid molecule according to 1) “plastidic” is depicted, or encoding a mitochondrial transit peptide, especially in case in column 6 of Table I, application no. 8, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 8, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 8, in the same line as of the respective nucleic acid molecule according to 1) “plastidic” is depicted, or encoding a mitochondrial transit peptide, especially in case in column 6 of Table I, application no. 8, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 8 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 8, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 8 (in the same line) and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 8, column 5 or 8, preferably shown in Table II A, application no. 8, column 5, or in Table II A, application no. 8, column 8, or in Table II B, application no. 8, column 8, or a homolog or a fragment thereof;
   (b) a nucleic acid molecule shown in Table I, application no. 8, column 5 or 8, preferably shown in Table I A, application no. 8, column 5, or in Table I A, application no. 8, column 8, or in Table I B, application no. 8, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 8, preferably shown in Table II A, application no. 8, column 5, or in Table II A, application no. 8, column 8, or in Table II B, application no. 8, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 8, preferably shown in Table I A, application no. 8, column 5, or in Table I A, application no. 8, column 8, or in Table I B, application no. 8, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 8;
   (i) a nucleic acid molecule encoding a polypeptide having the activity represented by a respective protein as depicted in column 5 of Table II, application no. 8;
   (j) a nucleic acid molecule comprising a polynucleotide which is obtained by amplifying a cDNA library or a genomic library using the primers in Table III, application no. 8, column 8;
   and
   (k) nucleic acid molecule which is obtainable by screening a suitable nucleic acid library, specially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising an expression cassette comprising
[0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 8, column 5 or 8, preferably shown in Table II A, application no. 8, column 5, or in Table II A, application no. 8, column 8, or in Table II B, application no. 8, column 8, or a homolog or a fragment thereof;
   (b) a nucleic acid molecule shown in Table I, application no. 8, column 5 or 8, preferably shown in Table I A, application no. 8, column 5, or in Table I A, application no. 8, column 8, or in Table I B, application no. 8, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 8, preferably shown in Table II A, application no. 8, column 5, or in Table II A, application no. 8, column 8, or in Table II B, application no. 8, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 8, preferably shown in Table I A, application no. 8, column 5, or in Table I A, application no. 8, column 8, or in Table I B, application no. 8, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 8;
   (i) a nucleic acid molecule encoding a polypeptide having the activity represented by a respective protein as depicted in column 5 of Table II, application no. 8;
   (j) a nucleic acid molecule comprising a polynucleotide which is obtained by amplifying a cDNA library or a genomic library using the primers in Table III, application no. 8, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      and, if desired
2) nucleic acid molecule encoding a transit peptide, preferably encoding a plastidal transit peptide or a mitochondrial transit peptide;
   which are operable linked;]
   or comprising or being transformed by a vector comprising
[1) a nucleic acid molecule selected from the group consisting of
   (a) a nucleic acid molecule encoding a polypeptide shown in Table II, application no. 8, column 5 or 8, preferably shown in Table II A, application no. 8, column 5, or in Table II A, application no. 8, column 8, or in Table II B, application no. 8, column 8, or a homolog or a fragment thereof;
   (b) a nucleic acid molecule shown in Table I, application no. 8, column 5 or 8, preferably shown in Table I A, application no. 8, column 5, or in Table I A, application no. 8, column 8, or in Table I B, application no. 8, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 8, preferably shown in Table II A, application no. 8, column 5, or in Table II A, application no. 8, column 8, or in Table II B, application no. 8, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 8, preferably shown in Table I A, application no. 8, column 5, or in Table I A, application no. 8, column 8, or in Table I B, application no. 8, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 8;
   (i) a nucleic acid molecule encoding a polypeptide having the activity represented by a respective protein as depicted in column 5 of Table II, application no. 8;
   (j) a nucleic acid molecule comprising a polynucleotide which is obtained by amplifying a cDNA library or a genomic library using the primers in Table III, application no. 8, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      whereby, preferably, the nucleic acid molecule according to 1 (a), (b), (c), (d), (e), (f), (g), (h), (i), (j) or (k) distinguishes over the sequence depicted in Table I A and/or I B, application no. 8, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 8, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 8, column 5 or 8, or the coding region thereof, respectively. In a further embodiment the nucleic acid molecule does not encode the polypeptide sequence shown in Table II A and/or II B, application no. 8, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 8, column 5 or 8 and does not encode a protein of the sequence shown in Table II A and/or II B, application no. 8, column 5 or 8. Accordingly, in one embodiment, the protein encoded by a sequence of a nucleic acid according to 1 (a), (b), (c), (d), (e), (f), (g), (h), (i), (j) or (k) does not consist of the sequence shown in Table I A and/or I B, application no. 8, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 8, column 5 or 8 but less than 100%, preferably less than 99.999%, 99.99%, 99.9%, 99%, 98%, 97%, 96% or 95% identical to the sequence shown in Table II A and/or II B, application no. 8, column 5 or 8;
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 8.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 8 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 8, in the same line as of the respective nucleic acid molecule according to 1) “plastidic” is depicted, or encoding a mitochondrial transit peptide, especially in case in column 6 of Table I, application no. 8, in the same line as of the respective nucleic acid molecule according to 1) “mitochondrial” is depicted.

In another embodiment thereof the expression cassette does not comprise 2) a nucleic acid molecule encoding a transit peptide, especially in case in column 6 of Table I, application no. 8, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 8.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the same line in column 5 of Table II, application no. 8, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 8, in the same line as of the respective nucleic acid molecule according to 1) “plastidic” is depicted, or encoding a mitochondrial transit peptide, especially in case in column 6 of Table I, application no. 8, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 8, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 8, in the same line as of the respective nucleic acid molecule according to 1) “plastidic” is depicted, or encoding a mitochondrial transit peptide, especially in case in column 6 of Table I, application no. 8, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 8, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 8, in the same line as of the respective nucleic acid molecule according to 1) “plastidic” is depicted, or encoding a mitochondrial transit peptide, especially in case in column 6 of Table I, application no. 8, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 8.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 8, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 8.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 8, in the same line as of the respective nucleic acid molecule according to 1) “plastidic” is depicted, or encoding a mitochondrial transit peptide, especially in case in column 6 of Table I, application no. 8, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 8, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 8, in the same line as of the respective nucleic acid molecule according to 1) “plastidic” is depicted, or encoding a mitochondrial transit peptide, especially in case in column 6 of Table I, application no. 8, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 8 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 8, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 8 and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

In an embodiment thereof the transgenic non-human organism is a microorganism selected from the group consisting of Charophyceae such as the genera Chara, Nitella e.g. the species Chara globularis, Chara vulgaris, Nitella flexilis, Chlorophyceae such as the genera Acrosiphonia, Spongomorpha, Urospora, Bryopsis, Pseudobryopsis, Trichosolen, Dichotomosiphon, Caulerpa, Rhipilia, Blastophysa, Avrainvillea, Chlorodesmis, Codium, Espera, Halicystis, Halimeda, Penicillus, Pseudocodium, Rhipiliopsis, Rhipocephalus, Tydemania, Udotea, Derbesia, Acrochaete, Aphanochaete, Bolbocoleon, Chaetobolus, Chaetonema, Chaetophora, Chlorotylium, Desmococcus, Draparnaldia, Draparnaldiopsis, Ectochaete, Endophyton, Entocladia, Epicladia, Internoretia, Microthamnion, Ochlochaete, Phaeophila, Pilinella, Pringsheimiella, Protoderma, Pseudendoclonium, Pseudodictyon, Pseudopringsheimia, Pseudulvella, Schizomeris, Stigeoclonium, Thamniochaete, Ulvella, Pilinia, Tellamia, Helicodictyon, Actidesmium, Ankyra, Characium, Codiolum, Sykidion, Keratococcus, Prototheca, Bracteacoccus Chlorococcum, Excentrosphaera, Hormidium, Oophila, Schroederia, Tetraedron, Trebouxia Chlorosarcinopsis, Gomphonitzschia, Coccomyxa, Dactylothece, Diógenes, Disporá, Gloeocystis, Mycanthococcus, Ourococcus, Coelastrum, Dicranochaete, Botryococcus, Dictyosphaerium, Dimorphococcus, Chlorochytrium, Kentrosphaera, Phyllobium, Gomontia, Hormotila, Euastropsis, Hydrodictyon, Pectodictyon, Pediastrum, Sorastrum, Tetrapedia, Acanthosphaera, Echinosphaerella, Echinosphaeridium, Errerella, Gloeoactinium, Golenkeniopsis Golenkinia, Micractinium, Ankistrodesmus, Chlorella, Chodatella, Closteriopsis, Cryocystis, Dactylococcus, Dematractum, Eremosphaera, Eutetramorus, Franceia, Glaucocystis, Gloeotaenium, Kirchneriella, Lagerheimiella, Monoraphidium, Nannochloris, Nephrochlamys, Nephrocytium, Oocystis, Oonephris, Pachycladon, Palmellococcus, Planktosphaeria, Polyedriopsis, Pseudoraciborskia, Quadrigula, Radiococcus, Rochiscia, Scotiella, Selanastrum, Thorakochloris, Treubaria, Trochiscia, Westella, Zoochlorella, Ostreobium, Phyllosiphon, Protosiphon, Rhodochytrium, Actinastrum, Coronastrum, Crucigenia, Dictymocystis, Enallax, Scenedesmus, Selenastrum, Tetradesmus, Tetrallantos, Tetrastrum, Chlorosarcina, Anadyomene, Valoniopsis,

Ventricaria, Basicladia, Chaetomorpha, Cladophora, Lola Pithophora Rhizoclonium Chaetosphaeridium, Conochaete, Coleochaete, Oligochaetophora, Polychaetophora, Cylindrocapsa, Gongrosira, Protococcus, Acetabularia, Batophora, Bornetella, Dasycladus, Halicoryne, Neomeris, Elakatothrix, Raphidonema, Microspora, Bulbochaete, Oedocladium, Oedogonium, Prasiola, Rosenvingiella, Schizogonium, Apjohnia, Chamaedoris, Cladophoropsis, Siphonocladus, Spongocladia, Boergesenia, Boodlea, Cystodictyon, Dictyosphaeria, Ernodesmis, Microdictyon, Struvea, Valonia, Sphaeroplea, Malleochloris, Stylosphaeridium, Gloeococcus, Palmella, Palmodictyon, Palmophyllum, Pseudospherocystis, Sphaerocystis, Urococcus, Apiocystis, Chaetopeltis, Gemellicystis, Paulschulzia, Phacomyxa, Pseudotetraspora, Schizochlamys, Tetraspora, Cephaleuros, Ctenocladus, Epibolium, Leptosira, Trentepohlia, Diplochaete, Monostroma, Binuclearia, Geminella, Klebsormidium, Planetonema, Radiofilum, Stichococcus, Ulothrix, Uronema, Blidingia, Capsosiphon, Chloropelta, Enteromorpha, Percursaria, Ulva, Ulvaria, Brachiomonas, Carteria, Chlainomonas, Chlamydomonas, Chlamydonephris, Chlorangium, Chlorogonium, Cyanidium, Fortiella, Glenomonas, Gloeomonas, Hyalogonium, Lobomonas Polytoma, Pyramichlamys, Scourfieldia, Smithsonimonas, Sphaerellopsis, Sphenochloris, Spirogonium, Collodictyon, Dunaliella, Haematococcus, Stephanosphaera, Coccomonas, Dysmorphococcus, Phacotus, Pteromonas, Thoracomonas, Wislouchiella, Mascherina, Pyrobotrys, Spondylomorum, Eudorina, Gonium, Oltmannsiella, Pandorina, Platydorina, Pleodorina,

Stephanoon, Volvox, Volvulina, Actinotaenium, Arthrodesmus, Bambusina Closterium, Cosmarium, Desmidium, Euastrum, Groenbladia, Hyalotheca, Micrasterias, Penium, Phymatodocis, Pleurotaenium, Sphaerozosma, Spinoclosterium, Spinocosmarium, Spondylosium, Staurastrum, Tetmemorus, Triploceras, Xanthidium, Cylindrocystis, Genicularia, Gonatozygon, Mesotaenium, Netrium, Roya, Spirotaenia, Cosmocladium, Debarya, Docidium, Euastridium, Hallasia, Mougeotia, Mougeotiopsis, Sirogonium, Spirogyra, Staurodesmus, Teilingia, Zygnema, Zygogonium, e.g. the species Caulerpa taxifolia, Prototheca wickerhamii, Ankistrodesmus falcatus, Chlorella ellipsoidea, Chlorella pyrenoidosa, Clorella sorokiniana, Chlorella vulgaris, Scenedesmus obliquus, Scenedesmus quadricauda, Selenastrum capricornutum, Selenastrum undecimnotata, Cladophora glomerata, Chlamydomonas eugametos, Chlamydomonas reinhardtii, Cyanidium caldarium, Dunaliella salina, Dunaliella tertiolecta, Euglena gracilis, Haematococcus pluvialis, Coniugatophyceae, Prasinophyceae Trebouxiophyceae, Ulvophyceae, Chlorodendraceae, Pedinomonadales, Halosphaeraceae, Pterospermataceae, Monomastigaceae, Pyramimonadaceae, Chlorodendraceae such as the genera Prasinocladus e.g. the species Prasinocladus ascus, Halosphaeraceae, Pedinomonadales, Pedinomonadaceae such as the genera Pedinomonas, Pterospermataceae such as the genera Pachysphaera, Pterosperma, Halosphaera, Pyramimonas, Bacillariophyceae, Chrysophyceae, Craspedophyceae, Euglenophyceae, Prymnesiophyceae, Phaeophyceae, Dinophyceae, Rhodophyceae, Xanthophyceae, Prasinophyceae such as the genera Nephroselmis, Prasinococcus, Scherffelia, Tetraselmis, Mantoniella, Ostreococcus e.g. the species Nephroselmis olivacea, Prasinococcus capsulatus, Scherffelia dubia, Tetraselmis chui, Tetraselmis suecica, Mantoniella squamata or Ostreococcus tauri.

In an embodiment thereof the transgenic non-human organism is a plant (or a part thereof), preferably a monocotyledonous or a dicotyledonous plant. Preferably the plant is selected from the group consisting of Anacardiaceae such as the genera Pistacia, Mangifera, Anacardium e.g. the species Pistacia vera [pistachios, Pistazie], Mangifer indica [Mango] or Anacardium occidentale [Cashew]; Asteraceae such as the genera Calendula, Carthamus, Centaurea, Cichorium, Cynara, Helianthus, Lactuca, Locusta, Tagetes, Valeriana e.g. the species Calendula officinalis [Marigold], Carthamus tinctorius [safflower], Centaurea cyanus [cornflower], Cichorium intybus [blue daisy], Cynara scolymus [Artichoke], Helianthus annus [sunflower], Lactuca sativa, Lactuca crispa, Lactuca esculenta, Lactuca scariola L. ssp. sativa, Lactuca scariola L. var. integrate, Lactuca scariola L. var. integrifolia, Lactuca sativa subsp. romana, Locusta communis, Valeriana locusta [lettuce], Tagetes lucida, Tagetes erecta or Tagetes tenuifolia [Marigold]; Apiaceae such as the genera Daucus e.g. the species Daucus carota [carrot]; Betulaceae such as the genera Corylus e.g. the species Corylus avellana or Corylus colurna [hazelnut]; Boraginaceae such as the genera Borago e.g. the species Borago officinalis [borage]; Brassicaceae such as the genera Brassica, Melanosinapis, Sinapis, Arabadopsis e.g. the species Brassica napus, Brassica rapa ssp. [canola, oilseed rape, turnip rape], Sinapis arvensis Brassica juncea, Brassica juncea var. juncea, Brassica juncea var. crispifolia, Brassica juncea var. foliosa, Brassica nigra, Brassica sinapioides, Melanosinapis communis [mustard], Brassica oleracea [fodder beet] or Arabidopsis thaliana; Bromeliaceae such as the genera Anana, Bromelia e.g. the species Anana comosus, Ananas ananas or Bromelia comosa [pineapple]; Caricaceae such as the genera Carica e.g. the species Carica papaya [papaya]; Cannabaceae such as the genera Cannabis e.g. the species Cannabis sative [hemp], Convolvulaceae such as the genera Ipomea, Convolvulus e.g. the species Ipomoea batatus, Ipomoea pandurata, Convolvulus batatas, Convolvulus tiliaceus, Ipomoea fastigiata, Ipomoea tiliacea, Ipomoea triloba or Convolvulus panduratus [sweet potato, Man of the Earth, wild potato], Chenopodiaceae such as the genera Beta, i.e. the species Beta vulgaris, Beta vulgaris var. altissima, Beta vulgaris var. Vulgaris, Beta maritima, Beta vulgaris var. perennis, Beta vulgaris var. conditiva or Beta vulgaris var. esculenta [sugar beet]; Cucurbitaceae such as the genera Cucubita e.g. the species Cucurbita maxima, Cucurbita mixta, Cucurbita pepo or Cucurbita moschata [pumpkin, squash]; Elaeagnaceae such as the genera Elaeagnus e.g. the species Olea europaea [olive]; Ericaceae such as the genera Kalmia e.g. the species Kalmia latifolia, Kalmia angustifolia, Kalmia microphylla, Kalmia polifolia, Kalmia occidentalis, Cistus chamaerhodendros or Kalmia lucida [American laurel, broad-leafed laurel, calico bush, spoon wood, sheep laurel, alpine laurel, bog laurel, western bog-laurel, swamp-laurel]; Euphorbiaceae such as the genera Manihot, Janipha, Jatropha, Ricinus e.g. the species Manihot utilissima, Janipha manihot, Jatropha manihot, Manihot aipil, Manihot dulcis, Manihot manihot, Manihot melanobasis, Manihot esculenta [manihot, arrowroot, tapioca, cassava] or Ricinus communis [castor bean, Castor Oil Bush, Castor Oil Plant, Palma Christi, Wonder Tree]; Fabaceae such as the genera Pisum, Albizia, Cathormion, Feuillea, Inga, Pithecolobium, Acacia, Mimosa, Medicajo, Glycine, Dolichos, Phaseolus, Soja e.g. the species Pisum sativum, Pisum arvense, Pisum humile [pea], Albizia berteriana, Albizia julibrissin, Albizia lebbeck, Acacia berteriana, Acacia littoralis, Albizia berteriana, Albizzia berteriana, Cathormion berteriana, Feuillea berteriana, Inga fragrans, Pithecellobium berterianum, Pithecellobium fragrans, Pithecolobium berterianum, Pseudalbizzia berteriana, Acacia julibrissin, Acacia nemu, Albizia nemu, Feuilleea julibrissin, Mimosa julibrissin, Mimosa speciosa, Sericanrda julibrissin, Acacia lebbeck, Acacia macrophylla, Albizia lebbek, Feuilleea lebbeck, Mimosa lebbeck, Mimosa speciosa [bastard logwood, silk tree, East Indian Walnut], Medicago sativa, Medicago falcata, Medicago varia [alfalfa], Glycine max [soybean], Dolichos soja, Glycine gracilis, Glycine hispida, Phaseolus max or Soja hispida; Geraniaceae such as the genera Pelargonium, Cocos, Oleum e.g. the species Cocos nucifera, Pelargonium grossularioides or Oleum cocois [coconut]; Gramineae such as the genera Saccharum e.g. the species Saccharum officinarum; Juglandaceae such as the genera Juglans, Wallia e.g. the species Juglans regia, Juglans ailanthifolia, Juglans sieboldiana, Juglans cinerea, Wallia cinerea, Juglans bixbyi, Juglans californica, Juglans hindsii, Juglans intermedia, Juglans jamaicensis, Juglans major, Juglans microcarpa, Juglans nigra or Wallia nigra [walnut, black walnut, common walnut, persian walnut, white walnut, butternut, black walnut]; Lauraceae such as the genera Persea, Laurus e.g. the species laurel Laurus nobilis [bay, laurel, bay laurel, sweet bay], Persea americana Persea americana, Persea gratissima or Persea persea [avocado]; Leguminosae such as the genera Arachis e.g. the species Arachis hypogaea [peanut]; Linaceae such as the genera Linum, Adenolinum e.g. the species Linum usitatissimum, Linum humile, Linum austriacum, Linum bienne, Linum angustifolium, Linum catharticum, Linum flavum, Linum grandiflorum, Adenolinum grandiflorum, Linum lewisii, Linum narbonense, Linum perenne, Linum perenne var. lewisii, Linum pratense or Linum trigynum [flax, linseed]; Lythrarieae such as the genera Punica e.g. the species Punica granatum [pomegranate]; Malvaceae such as the genera Gossypium e.g. the species Gossypium hirsutum, Gossypium arboreum, Gossypium barbadense, Gossypium herbaceum or Gossypium thurberi [cotton]; Musaceae such as the genera Musa e.g. the species Musa nana, Musa acuminata, Musa paradisiaca, Musa spp. [banana]; Onagraceae such as the genera Camissonia, Oenothera e.g. the species Oenothera biennis or Camissonia brevipes [primrose, evening primrose]; Palmae such as the genera Elacis e.g. the species Elaeis guineensis [oil plam]; Papaveraceae such as the genera Papaver e.g. the species Papaver orientale, Papaver rhoeas, Papaver dubium [poppy, oriental poppy, corn poppy, field poppy, shirley poppies, field poppy, long-headed poppy, long-pod poppy]; Pedaliaceae such as the genera Sesamum e.g. the species Sesamum indicum [sesame]; Piperaceae such as the genera Piper, Artanthe, Peperomia, Steffensia e.g. the species Piper aduncum, Piper amalago, Piper angustifolium, Piper auritum, Piper betel, Piper cubeba, Piper longum, Piper nigrum, Piper retrofractum, Artanthe adunca, Artanthe elongata, Peperomia elongata, Piper elongatum, Steffensia elongata. [Cayenne pepper, wild pepper]; Poaceae such as the genera Hordeum, Secale, Avena, Sorghum, Andropogon, Holcus, Panicum, Oryza, Zea, Triticum e.g. the species Hordeum vulgare, Hordeum jubatum, Hordeum murinum, Hordeum secalinum, Hordeum distichon Hordeum aegiceras, Hordeum hexastichon, Hordeum hexastichum, Hordeum irregulare, Hordeum sativum, Hordeum secalinum [barley, pearl barley, foxtail barley, wall barley, meadow barley], Secale cereale [rye], Avena sativa, Avena fatua, Avena byzantina, Avena fatua var. sativa, Avena hybrida [oat], Sorghum bicolor, Sorghum halepense, Sorghum saccharatum, Sorghum vulgare, Andropogon drummondii, Holcus bicolor, Holcus sorghum, Sorghum aethiopicum, Sorghum arundinaceum, Sorghum caffrorum, Sorghum cernuum, Sorghum dochna, Sorghum drummondii, Sorghum durra, Sorghum guineense, Sorghum lanceolatum, Sorghum nervosum, Sorghum saccharatum, Sorghum subglabrescens, Sorghum verticilliflorum, Sorghum vulgare, Holcus halepensis, Sorghum miliaceum millet, Panicum militaceum [Sorghum, millet], Oryza sativa, Oryza latifolia [rice], Zea mays [corn, maize] Triticum aestivum, Triticum durum, Triticum turgidum, Triticum hybernum, Triticum macha, Triticum sativum or Triticum vulgare [wheat, bread wheat, common wheat], Proteaceae such as the genera Macadamia e.g. the species Macadamia intergrifolia [macadamia]; Rubiaceae such as the genera Coffea e.g. the species Cofea spp., Coffea arabica, Coffea canephora or Coffea liberica [coffee]; Scrophulariaceae such as the genera Verbascum e.g. the species Verbascum blattaria, Verbascum chaixii, Verbascum densiflorum, Verbascum lagurus, Verbascum longifolium, Verbascum lychnitis, Verbascum nigrum, Verbascum olympicum, Verbascum phlomoides, Verbascum phoenicum, Verbascum pulverulentum or Verbascum thapsus [mullein, white moth mullein, nettle-leaved mullein, dense-flowered mullein, silver mullein, long-leaved mullein, white mullein, dark mullein, greek mullein, orange mullein, purple mullein, hoary mullein, great mullein]; Solanaceae such as the genera Capsicum, Nicotiana, Solanum, Lycopersicon e.g. the species Capsicum annuum, Capsicum annuum var. glabriusculum, Capsicum frutescens [pepper], Capsicum annuum [paprika], Nicotiana tabacum, Nicotiana alata, Nicotiana attenuata, Nicotiana glauca, Nicotiana langsdorffii, Nicotiana obtusifolia, Nicotiana quadrivalvis, Nicotiana repanda, Nicotiana rustica, Nicotiana sylvestris [tobacco], Solanum tuberosum [potato], Solanum melongena [egg-plant] (Lycopersicon esculentum, Lycopersicon lycopersicum, Lycopersicon pyriforme, Solanum integrifolium or Solanum lycopersicum [tomato]; Sterculiaceae such as the genera Theobroma e.g. the species Theobroma cacao [cacao]; Theaceae such as the genera Camellia e.g. the species Camellia sinensis) [tea]

Particular preference is given to transgenic crop plants such as by way of example barley, wheat, rye, oats, corn, soybean, rice, cotton, sugar beet, oilseed rape and canola, sunflower, flax, hemp, thistle, potatoes, tobacco, tomatoes, tapioca, cassava, arrowroot, alfalfa, lettuce and the various tree, nut and vine species; especial preference is given to monocotyledonous crop plants like corn, wheat or rice; in another embodiment special preference is given to dicotyledonous crop plants like soy bean, oil seed rape (including canola and winter oil seed rape), cotton.

The term “transgenic plants” used in accordance with the invention also refers to the progeny of a transgenic plant, for example the T₁, T₂, T₃ and subsequent plant generations or the BC₁, BC₂, BC₃ and subsequent plant generations. Thus, the transgenic plants according to the invention can be raised and selfed or crossed with other individuals in order to obtain further transgenic plants according to the invention. Transgenic plants may also be obtained by propagating transgenic plant cells vegetatively. The present invention also relates to transgenic plant material, which can be derived from a transgenic plant population according to the invention. Such material includes plant cells and certain tissues, organs and parts of plants in all their manifestations, such as seeds, leaves, anthers, fibers, tubers, roots, root hairs, stems, embryo, calli, cotyledons, petioles, harvested material, plant tissue, reproductive tissue, pollen, and cell cultures, which are derived from the actual transgenic plant and/or can be used for bringing about the transgenic plant. Any transformed plant obtained according to the invention can be used in a conventional breeding scheme or in vitro plant propagation to produce more transformed plants with the same characteristics and/or can be used to introduce the same characteristic in other varieties of the same or related species. Such plants are also part of the invention. Seeds obtained from the transformed plants genetically also contain the same characteristic and are part of the invention. As mentioned before, the present invention is in principle applicable to any plant and crop that can be transformed with any of the transformation method known to those skilled in the art.

An “isolated” nucleic acid molecule is one that is substantially separated from other nucleic acid molecules, which are present in the natural source of the nucleic acid. That means other nucleic acid molecules are present in an amount less than 5% based on weight of the amount of the desired nucleic acid, preferably less than 2% by weight, more preferably less than 1% by weight, most preferably less than 0.5% by weight. Preferably, an “isolated” nucleic acid is free of some of the sequences that naturally flank the nucleic acid (i.e., sequences located at the 5′ and 3′ ends of the nucleic acid) in the genomic DNA of the non-human organism from which the nucleic acid is derived. For example, in various embodiments, the FCRP encoding nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be free from some of the other cellular material with which it is naturally associated, or culture medium when produced by recombinant techniques, or chemical precursors or other chemicals when chemically synthesized.

A nucleic acid molecule of the present invention, e.g., a nucleic acid molecule encoding a FCRP, especially the coding region thereof, or a portion thereof which confers the production or increased production of the fine chemical, can be isolated using standard molecular biological techniques and the sequence information provided herein. For example, an A. thaliana FCRP encoding cDNA can be isolated from a A. thaliana c-DNA library or a E. coli, Saccharomyces cerevisiae, Synechocystis sp., Brassica napus, Glycine max, Zea mays or Oryza sativa FCRP encoding cDNA can be isolated from a E. coli, Saccharomyces cerevisiae, Synechocystis sp., Brassica napus, Glycine max, Zea mays or Oryza sativa c-DNA library respectively using all or portion of one of the respective sequences. Moreover, a nucleic acid molecule encompassing all or a portion of one of the sequences of Table I can be isolated by the polymerase chain reaction using oligonucleotide primers designed based upon this sequence. For example, mRNA can be isolated from plant cells (e.g., by the guanidiniumthiocyanate extraction procedure of Chirgwin et al., Biochemistry 18, 5294 (1979)) and cDNA can be prepared using reverse transcriptase (e.g., Moloney MLV reverse transcriptase, available from Gibco/BRL, Bethesda, MD; or AMV reverse transcriptase, available from Seikagaku America, Inc., St. Petersburg, Fla.). Synthetic oligonucleotide primers for polymerase chain reaction amplification can be designed based upon one of the nucleotide sequences shown in Table I. A nucleic acid molecule of the invention can be amplified using cDNA or, alternatively, genomic DNA, as a template and appropriate oligonucleotide primers according to standard PCR amplification techniques. The nucleic acid molecule so amplified can be cloned into an appropriate vector and characterized by DNA sequence analysis. Furthermore, oligonucleotides corresponding to a FCRP encoding nucleotide sequence can be prepared by standard synthetic techniques, e.g., using an automated DNA synthesizer.

In an embodiment, an isolated nucleic acid molecule of the invention comprises one of the nucleic acid molecules as shown in Table I, especially the coding region thereof, and if desired, as well as a 5′ untranslated sequence and 3′ untranslated sequence.

Moreover, the nucleic acid molecule of the invention can comprise only a portion of the coding region of one of the nucleic acid molecule of Table I, for example, a fragment which can be used as a probe or primer or a fragment encoding a biologically active portion of a FCRP.

Portions of proteins encoded by the FCRP encoding nucleic acid molecules of the invention are preferably biologically active portions described herein. As used herein, the term “biologically active portion of” a FCRP is intended to include a portion, e.g. a domain/motif, of the protein being responsible for the ability of the protein to enable the production or increased production of the fine chemical. To determine whether a FCRP, or a biologically active portion thereof, results in a production or an increased production of the fine chemical in the non-human organism, like a microorganism or a plant, an analysis of the non-human organism comprising the FCRP may be performed. Such analysis methods are well known to those skilled in the art, as detailed in the Examples. More specifically, nucleic acid fragments encoding biologically active portions of a FCRP can be prepared by isolating a portion of one of the sequences of the nucleic acid of Table I, e.g. expressing the encoded portion of the FCRP or peptide (e.g. by recombinant expression in vitro) and assessing the activity of the encoded portion of the FCRP or peptide.

Biologically active portions of a FCRP are encompassed by the present invention and include peptides comprising amino acid sequences derived from the amino acid sequence of a FCRP encoding gene, or the amino acid sequence of a protein homologous to a FCRP, which include fewer amino acids than a full length FCRP or the full length protein which is homologous to a FCRP, and exhibits at least some enzymatic or biological activity of a FCRP. Typically, biologically active portions (e.g., peptides which are, for example, 5, 10, 15, 20, 30, 35, 36, 37, 38, 39, 40, 50, 75, 100, 125, 150 or more amino acids in length) comprise a domain or motif with at least one activity of a FCRP. Moreover, other biologically active portions in which other regions of the protein are deleted, can be prepared by recombinant techniques and evaluated for one or more of the activities described herein. Preferably, the biologically active portions of a FCRP include one or more selected domains/motifs or portions thereof having biological activity.

The term “biological active portion” or “biological activity” means a polypeptide as depicted in the respective line in Table II, application no. 8, column 3 or a portion of said polypeptide which still has at least 10% or 20%, preferably 30%, 40%, 50% or 60%, especially preferably 70%, 75%, 80%, 90% or 95% of the enzymatic or biological activity of the natural or starting enzyme or protein.

In the process according to the invention nucleic acid sequences or molecules can be used, which, if appropriate, contain synthetic, non-natural or modified nucleotide bases, which can be incorporated into DNA or RNA. Said synthetic, non-natural or modified bases can for example increase the stability of the nucleic acid molecule outside or inside a cell. The nucleic acid molecules of the invention can contain the same modifications as aforementioned.

As used in the present context the term “nucleic acid molecule” may also encompass the untranslated sequence or molecule located at the 3′ and at the 5′ end of the coding gene region, for example at least 500, preferably 200, especially preferably 100, nucleotides of the sequence upstream of the 5′ end of the coding region and at least 100, preferably 50, especially preferably 20, nucleotides of the sequence downstream of the 3′ end of the coding gene region. It is often advantageous only to choose the coding region for cloning and expression purposes.

Preferably, the nucleic acid molecule used in the process according to the invention or the nucleic acid molecule of the invention is an isolated nucleic acid molecule. In one embodiment, the nucleic acid molecule of the invention is the nucleic acid molecule used in the process of the invention.

The nucleic acid molecules used in the process, for example the polynucleotide of the invention or of a part thereof can be isolated using molecular-biological standard techniques and the sequence information provided herein. Also, for example a homologous sequence or homologous, conserved sequence regions at the DNA or amino acid level can be identified with the aid of comparison algorithms. The former can be used as hybridization probes under standard hybridization techniques (for example those described in Sambrook et al., “Molecular Cloning: A Laboratory Manual” 2^{nd} ed., Cold Spring Harbor Laboratory, Cold Spring Harbor Laboratory Press, Cold Spring Harbor, N.Y., 1989) for isolating further nucleic acid sequences useful in this process.

Synthetic oligonucleotide primers for the amplification, e.g. as shown in the respective line in Table III, application no. 8, column 8, by means of polymerase chain reaction can be generated on the basis of a sequence shown herein, for example the sequence shown in the respective line in Table I, application no. 8, columns 5 or 8, or the sequences derived from Table II, application no. 8, columns 5 or 8, respectively.

Moreover, it is possible to identify a conserved region of a protein from various organism by carrying out protein sequence alignments with the polypeptide encoded by the nucleic acid molecules of the present invention, in particular with the sequences encoded by the nucleic acid molecule shown in the respective line in column 5 or 8 of Table I, application no. 8, from which conserved regions, and in turn, degenerate primers can be derived. Conserved regions are those, which show a very little variation in the amino acid in one particular position of several homologs from different origin. The consensus sequence and polypeptide motifs shown in the respective line in column 8 of Table IV, application no. 8, are derived from such alignments of homologous proteins.

In an embodiment of the present invention, in the method of the present invention the activity of a polypeptide comprising or consisting of a consensus sequence or at least one polypeptide motif shown in the respective line in Table IV, application no. 8, column 8, is increased. In another embodiment thereof, the method of the present invention relates to a polypeptide comprising or consisting of a consensus sequence shown in the respective line in Table IV, application no. 8, column 8 or comprising at least one polypeptide motif shown in the respective line in Table IV, application no. 8, column 8, whereby not more than 20, 15, 10, 9, 8, 7, 6, 5 4, 3, 2 or 1, or 0 of the amino acid positions indicated can be replaced by any amino acid. In another embodiment thereof, the method of the present invention relates to a polypeptide comprising or consisting of a consensus sequence shown in the respective line in Table IV, application no. 8, column 8 or comprising at least one polypeptide motif shown in the respective line in Table IV, application no. 8, column 8, whereby not more than 15%, 10%, 5%, 4%, 3%, 2% or 1%, or 0% of the amino acid positions defined as distinct specific amino acids are/is replaced by another amino acid. In another embodiment thereof, the method of the present invention relates to a polypeptide comprising or consisting of a consensus sequence shown in the respective line in Table IV, application no. 8, column 8 or comprising at least one polypeptide motif shown in the respective line in Table IV, application no. 8, column 8, whereby not more than 20, 15, 10, 9, 8, 7, 6, 5, 4, 3, 2 or 1, or 0 amino acids are inserted into a consensus sequence or polypeptide motif.

In an embodiment of the present invention, in the method of the present invention the activity of a polypeptide comprising or consisting of a consensus sequence or at least one polypeptide motif shown in the respective line in Table IV, application no. 8, column 8, is increased, whereby said polypeptide distinguishes over the sequence depicted in Table II, application no. 8, columns 5 or 8 by one or more amino acids.

In one embodiment, said polypeptide of the invention distinguishes over the sequence shown in the respective line in Table II, application no. 8, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II B, application no. 8, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in Table II, application no. 8, columns 5 or 8.

The consensus sequence was derived from a multiple alignment of the sequences as listed in a single line of Table II. The amino acids are given in their three letter code. In case a specific amino acid is indicated this specific amino acid is conserved in at least 80% of the aligned proteins whereas the three letter code XAA stands for amino acids, which are not conserved in at least 80% of the aligned sequences. The consensus sequence starts with the first conserved amino acid in the alignment, and ends with the last conserved amino acid in the alignment of the investigated sequences.

Patterns had to match at least 80% of the investigated proteins. Conserved patterns were identified with the software tool MEME version 3.5.1 or manually by using first standard prosite annotation. The number of given x indicates the distances between conserved amino acid residues (given in the one letter code), e.g. (1) the pattern Y-x(21,23)-F means that conserved tyrosine and phenylalanine residues in the alignment are separated from each other by minimum 21 and maximum 23 amino acid residues in the alignment of all investigated sequences, (2) the pattern Y-x(21,23)-[FW] means that a conserved tyrosine is separated by minimum 21 and maximum 23 amino acid residues from either a phenylalanine or tryptophane. However, these patterns have been “translated” into the WIPO standard 25. MEME was developed by Bailey Timothy L. and Elkan Charles, Dept. of Computer Science and Engineering, University of California, San Diego, USA and is described by Bailey Timothy L. and Elkan Charles (Proceedings of the Second International Conference on Intelligent Systems for Molecular Biology, pp. 28-36, AAAI Press, Menlo Park, Calif., 1994). The source code for the stand-alone program is public available from the San Diego Supercomputer centre (http://meme.sdsc.edu). For identifying common motifs in all sequences with the software tool MEME, the following settings were used:—maxsize 500000,—nmotifs 15,—evt 0.001,—maxw 60, Distance 1e−3,—minsites number of sequences used for the analysis. Input sequences for MEME were non-aligned sequences in Fasta format. Other parameters were used in the default settings in this software version. Prosite patterns for conserved domains were generated with the software tool Pratt version 2.1 or manually. Pratt was developed by Jonassen Inge, Dept. of Informatics, University of Bergen, Norway and is described by Jonassen et al. (Jonassen I., Collins J. F. and Higgins D. G., Protein Science 4, 1587 (1995); Jonassen I., Efficient discovery of conserved patterns using a pattern graph, Submitted to CABIOS February 1997). The source code (ANSI C) for the stand-alone program is public available, e.g. at established Bioinformatic centers like EBI (European Bioinformatics Institute). For generating patterns with the software tool Pratt, following settings were used: PL (max Pattern Length): 100, PN (max Nr of Pattern Symbols): 100, PX (max Nr of consecutive x's): 30, FN (max Nr of flexible spacers): 5, FL (max Flexibility): 30, FP (max Flex.Product): 10, ON (max number patterns): 50. Input sequences for

Pratt were distinct regions of the protein sequences exhibiting high similarity as identified from software tool MEME. The minimum number of sequences, which have to match the generated patterns (CM, min Nr of Seqs to Match) was set to at least 80% of the provided sequences. Parameters not mentioned here were used in their default settings. The Prosite patterns of the conserved domains can be used to search for protein sequences matching this pattern. Various established Bioinformatic centres provide public internet portals for using those patterns in database searches (e.g. PIR (Protein Information Resource, located at Georgetown University Medical Center) or ExPASy (Expert Protein Analysis System)). Alternatively, stand-alone software is available, like the program Fuzzpro, which is part of the EMBOSS software package. For example, the program Fuzzpro not only allows searching for an exact pattern-protein match but also allows setting various ambiguities in the performed search.

The alignment was performed with the software ClustalW (version 1.83) and is described by Thompson et al. (Nucleic Acids Research 22, 4673 (1994)). The source code for the stand-alone program is public available from the European Molecular Biology Laboratory; Heidelberg, Germany. The analysis was performed using the default parameters of ClustalW v1.83 (gap open penalty: 10.0; gap extension penalty: 0.2; protein matrix: Gonnet; protein/DNA endgap: −1; protein/DNA gapdist: 4).

Degenerated primers can then be utilized by PCR for the amplification of fragments of novel proteins having above-mentioned activity, e.g. conferring the production or the increased production of the fine chemical as compared to a corresponding, non-transformed, wild type cell, or non-human organism, like a plant cell or a plant or a part thereof, after generating or increasing the expression or activity or having the activity of a protein as shown in the respective line in Table II, column 3 or further functional homologs of the polypeptide of the invention from other non-human organisms.

These fragments can then be utilized as hybridization probe for isolating the complete gene sequence. As an alternative, the missing 5′ and 3′ sequences can be isolated by means of RACE-PCR. A nucleic acid molecule according to the invention can be amplified using cDNA or, as an alternative, genomic DNA as template and suitable oligonucleotide primers, following standard PCR amplification techniques. The nucleic acid molecule amplified thus can be cloned into a suitable vector and characterized by means of DNA sequence analysis. Oligonucleotides, which correspond to one of the nucleic acid molecules used in the process, can be generated by standard synthesis methods, for example using an automatic DNA synthesizer.

Nucleic acid molecules which are advantageously for the process according to the invention can be isolated based on their homology to the nucleic acid molecules disclosed herein using the sequences or part thereof as or for the generation of a hybridization probe and following standard hybridization techniques under stringent hybridization conditions. In this context, it is possible to use, for example, isolated one or more nucleic acid molecules of at least 15, 20, 25, 30, 35, 40, 50, 60 or more nucleotides, preferably of at least 15, 20 or 25 nucleotides in length which hybridize under stringent conditions with the above-described nucleic acid molecules, in particular with those which encompass a nucleotide sequence of the nucleic acid molecule used in the process of the invention or encoding a protein used in the invention or of the nucleic acid molecule of the invention. Nucleic acid molecules with 30, 50, 100, 250 or more nucleotides may also be used.

The term “homology” means that the respective nucleic acid molecules or the encoded proteins are functionally and/or structurally equivalent. The nucleic acid molecules that are homologous to the nucleic acid molecules described above and that are derivatives of said nucleic acid molecules are, for example, variations of said nucleic acid molecules which represent modifications having the same biological function, in particular encoding proteins with the same or substantially the same biological function. They may be naturally occurring variations, such as sequences from other plant varieties or species, or mutations. These mutations may occur naturally or may be obtained by mutagenesis techniques. The allelic variations may be naturally occurring allelic variants as well as synthetically produced or genetically engineered variants. Structurally equivalents can, for example, be identified by testing the binding of said polypeptide to antibodies or computer based predictions. Structurally equivalents have the similar immunological characteristic, e.g. comprise similar epitopes.

By “hybridizing” it is meant that such nucleic acid molecules hybridize under conventional hybridization conditions, preferably under stringent conditions such as described by, e.g., Sambrook (“Molecular Cloning; A Laboratory Manual”, 2nd edition, Cold Spring Harbor Laboratory Press, Cold Spring Harbor, N.Y. (1989)) or in “Current Protocols in Molecular Biology”, John Wiley & Sons, N.Y. (1989), chapter 6.3.1-6.3.6.)

According to the invention, DNA as well as RNA molecules of the corresponding nucleic acid molecules of the invention can be used as probes. Further, as template for the identification of functional homologues Northern blot assays as well as Southern blot assays can be performed. The Northern blot assay advantageously provides further information about the expressed gene product: e.g. expression pattern, occurrence of processing steps, like splicing and capping, etc. The Southern blot assay provides additional information about the chromosomal localization and organization of the gene being related to the nucleic acid molecule of the invention.

A preferred, non-limiting example of stringent hybridization conditions are hybridizations in 6×sodium chloride/sodium citrate (=SSC) at approximately 45° C., followed by one or more wash steps in 0.2×SSC, 0.1% SDS at 50 to 65° C., for example at 50° C., 55° C. or 60° C. The skilled worker knows that these hybridization conditions differ as a function of the type of the nucleic acid and, for example when organic solvents are present, with regard to the temperature and concentration of the buffer. The temperature under “standard hybridization conditions” differs for example as a function of the type of the nucleic acid between 42° C. and 65° C., preferably between 45° C. and 50° C. in an aqueous buffer with a concentration of 0.1×, 0.5×, 1×, 2×, 3×, 4× or 5×SSC (pH 7.2). If organic solvent(s) is/are present in the abovementioned buffer, for example 50% formamide, the temperature under standard conditions is approximately 40° C., 42° C. or 45° C. The hybridization conditions for DNA:DNA hybrids are preferably for example 0.1×SSC and 20° C., 25° C., 30° C., 35° C., 40° C. or 45° C., preferably between 30° C. and 45° C. The hybridization conditions for DNA:RNA hybrids are preferably for example 0.1×SSC and 30° C., 35° C., 40° C., 45° C., 50° C. or 55° C., preferably between 45° C. and 55° C. The above-mentioned hybridization temperatures are determined for example for a nucleic acid approximately 100 by (=base pairs) in length and a G+C content of 50% in the absence of formamide. The skilled worker knows to determine the hybridization conditions required with the aid of textbooks, for example the ones mentioned above, or from the following textbooks: Sambrook et al., “Molecular Cloning”, Cold Spring Harbor Laboratory, 1989; Hames and Higgins (ed.), “Nucleic Acids Hybridization: A Practical Approach”, IRL Press at Oxford University Press, Oxford, 1985; Brown (ed.), “Essential Molecular Biology: A Practical Approach”, IRL Press at Oxford University Press, Oxford, 1991.

A further example of one such stringent hybridization condition is hybridization at 4×SSC at 65° C., followed by a washing in 0.1×SSC at 65° C. for one hour. Alternatively, an exemplary stringent hybridization condition is in 50% formamide, 4×SSC at 42° C. Further, the conditions during the wash step can be selected from the range of conditions delimited by lowstringency conditions (approximately 2×SSC at 50° C.) and high-stringency conditions (approximately 0.2×SSC at 50° C., preferably at 65° C.) (20×SSC : 0.3 M sodium citrate, 3 M NaCl, pH 7.0). In addition, the temperature during the wash step can be raised from low-stringency conditions at room temperature, approximately 22° C., to higher-stringency conditions at approximately 65° C. Both of the parameters salt concentration and temperature can be varied simultaneously, or else one of the two parameters can be kept constant while only the other is varied. Denaturants, for example formamide or SDS, may also be employed during the hybridization. In the presence of 50% formamide, hybridization is preferably effected at 42° C. Relevant factors like 1) length of treatment, 2) salt conditions, 3) detergent conditions, 4) competitor DNAs, 5) temperature and 6) probe selection can be combined case by case so that not all possibilities can be mentioned herein.

Thus, in a preferred embodiment, Northern blots are prehybridized with RothiHybri-Quick buffer (Roth, Karlsruhe) at 68° C. for 2 h. Hybridization with radioactive labeled probe is done overnight at 68° C. Subsequent washing steps are performed at 68° C. with 1×SSC. For Southern blot assays the membrane is prehybridized with Rothi-Hybri-Quick buffer (Roth, Karlsruhe) at 68° C. for 2 h. The hybridization with radioactive labeled probe is conducted over night at 68° C. Subsequently the hybridization buffer is discarded and the filter shortly washed using 2×SSC; 0.1% SDS. After discarding the washing buffer new 2×SSC; 0.1% SDS buffer is added and incubated at 68° C. for 15 minutes. This washing step is performed twice followed by an additional washing step using 1×SSC; 0.1% SDS at 68° C. for 10 min.

Some examples of conditions for DNA hybridization (Southern blot assays) and wash step are shown herein below:

(1) Hybridization conditions can be selected, for example, from the following conditions:
(a) 4×SSC at 65° C.,
(b) 6×SSC at 45° C.,
(c) 6×SSC, 100 mg/ml denatured fragmented fish sperm DNA at 68° C.,
(d) 6×SSC, 0.5% SDS, 100 mg/ml denatured salmon sperm DNA at 68° C.,
(e) 6×SSC, 0.5% SDS, 100 mg/ml denatured fragmented salmon sperm DNA, 50% formamide at 42° C.,
(f) 50% formamide, 4×SSC at 42° C.,
(g) 50% (v/v) formamide, 0.1% bovine serum albumin, 0.1% Ficoll, 0.1% polyvinylpyrrolidone, 50 mM sodium phosphate buffer pH 6.5, 750 mM NaCl, 75 mM sodium citrate at 42° C.,
(h) 2× or 4×SSC at 50° C. (low-stringency condition), or
(i) 30 to 40% formamide, 2× or 4×SSC at 42° C. (low-stringency condition).
   (2) Wash steps can be selected, for example, from the following conditions:
(a) 0.015 M NaCl/0.0015 M sodium citrate/0.1% SDS at 50° C.,
(b) 0.1×SSC at 65° C.,
(c) 0.1×SSC, 0.5% SDS at 68° C.,
(d) 0.1×SSC, 0.5% SDS, 50% formamide at 42° C.,
(e) 0.2×SSC, 0.1% SDS at 42° C.,
(f) 2×SSC at 65° C. (low-stringency condition),

Polypeptides having above-mentioned activity, i.e. conferring the production or the increased production of the fine chemical linoleic acid as compared to a corresponding, e.g. non-transformed, wild type non-human organism, like a microorganism or a plant cell, plant or part thereof, can be encoded by other DNA sequences which hybridize to the sequences shown in the respective line in Table I, application no. 8, columns 5 and 8, preferably the coding region thereof, at least under relaxed hybridization conditions and which encode the expression of polypeptides conferring the production or the increased production of the respective fine chemical linoleic acid as compared to a corresponding, e.g. non-transformed, wild type non-human organism, like a microorganism or a plant cell, plant or part thereof.

Further, some applications have to be performed at low stringency hybridization conditions, without any consequences for the specificity of the hybridization. For example, a Southern blot analysis of total DNA could be probed with a nucleic acid molecule of the present invention and washed at low stringency (55° C. in 2×SSPE, 0.1% SDS). The hybridization analysis could reveal a simple pattern of only genes encoding polypeptides of the present invention or used in the process of the invention, e.g. having the herein-mentioned activity of conferring the production or the increased production of the fine chemical as compared to a corresponding, e.g. non-transformed, wild type non-human organism, like a microorganism or a plant cell, plant or part thereof. A further example of such low-stringent hybridization conditions is 4×SSC at 50° C. or hybridization with 30 to 40% formamide at 42° C. Such molecules comprise those which are fragments, analogues or derivatives of the polypeptide of the invention or used in the process of the invention and differ, for example, by way of amino acid and/or nucleotide deletion(s), insertion(s), substitution (s), addition(s) and/or recombination (s) or any other modification(s) known in the art either alone or in combination from the above-described amino acid sequences or their underlying nucleotide sequence(s). However, it is preferred to use high stringency hybridization conditions.

Hybridization should advantageously be carried out with fragments of at least 5, 10, 15, 20, 25, 30, 35 or 40 bp, advantageously at least 50, 60, 70 or 80 bp, preferably at least 90, 100 or 110 bp. Most preferably are fragments of at least 15, 20, 25 or 30 bp. Preferably are also hybridizations with at least 100 by or 200 bp, very especially preferably at least 400 by in length. In an especially preferred embodiment, the hybridization should be carried out with the entire nucleic acid sequence with conditions described above.

The terms “fragment”, “fragment of a sequence” or “part of a sequence” mean a truncated sequence of the original sequence referred to. The truncated sequence (nucleic acid or protein sequence) can vary widely in length; the minimum size being a sequence of sufficient size to provide a sequence with at least a comparable function and/or activity of the original sequence or molecule referred to, or hybridizing with the nucleic acid molecule of the invention or used in the process of the invention under stringent conditions, while the maximum size is not critical. In some applications, the maximum size usually is not substantially greater than that required to provide the desired activity and/or function(s) of the original sequence.

Typically, the truncated amino acid sequence or molecule will range from about 5 to about 310 amino acids in length. More typically, however, the sequence will be a maximum of about 250 amino acids in length, preferably a maximum of about 200 or 100 amino acids. It is usually desirable to select sequences of at least about 10, 12 or 15 amino acids, up to a maximum of about 20 or 25 amino acids.

The term “epitope” relates to specific immunoreactive sites within an antigen, also known as antigenic determinates. These epitopes can be a linear array of monomers in a polymeric composition—such as amino acids in a protein—or consist of or comprise a more complex secondary or tertiary structure. Those of skill will recognize that immunogens (i.e. substances capable of eliciting an immune response) are antigens; however, some antigen, such as haptens, are not immunogens but may be made immunogenic by coupling to a carrier molecute. The term “antigen” includes references to a substance to which an antibody can be generated and/or to which the antibody is specifically immunoreactive.

In an embodiment the present invention relates to an epitope of the polypeptide of the present invention or used in the process of the present invention and conferring the production or the increased production of the fine chemical as compared to a corresponding, e.g. non-transformed, wild type non-human organism, like a microorganism or a plant cell, plant or part thereof.

The term “one or several amino acids” relates to at least one amino acid but not more than that number of amino acids, which would result in a homology of below 50% identity. Preferably, the identity is more than 70%, 80%, 85%, 90%, 91%, 92%, 93%, 94%, 95%, 96%, 97%, 98%, 99% or 99.5% identity.

Further, the nucleic acid molecule of the invention comprises a nucleic acid molecule, which is a complement of one of the nucleotide sequences of above-mentioned nucleic acid molecules or a portion thereof. A nucleic acid molecule or its sequence which is complementary to one of the nucleotide molecules or sequences shown in the respective line in Table I, application no. 8, columns 5 or 8, preferably the coding region thereof, homologs or fragments thereof, is one which is sufficiently complementary to one of the nucleotide molecules or sequences shown in the respective line in Table I, application no. 8, columns 5 or 8, preferably the coding region thereof, homologs or fragments thereof, such that it can hybridize to one of the nucleotide sequences shown in Table I, application no. 8, columns 5 or 8, preferably the coding region thereof, homologs or fragments thereof, thereby forming a stable duplex. Preferably, the hybridization is performed under stringent hybridization conditions. However, a complement of one of the herein disclosed sequences is preferably a sequence complement thereto according to the base pairing of nucleic acid molecules well known to the skilled person. For example, the bases A and G undergo base pairing with the bases T and U or C, resp. and visa versa. Modifications of the bases can influence the base-pairing partner.

The nucleic acid molecule of the invention comprises a nucleotide sequence which is at least about 30%, 35%, 40%, 45%, 50%, 55%, 60%, 65%, 70%, 75%, 80%, 90%, 95%, 96%, 97%, 98%, 99%, 99.5% or more homologous to a nucleotide sequence shown in Table I, application no. 8, columns 5 or 8, preferably the coding region thereof, homologs or fragments thereof, and preferably has above-mentioned activity, in particular conferring the production or the increased production of the fine chemical linoleic acid, respectively, after increasing the activity or an activity of a gene as shown in the respective line in Table I or of a gene product, e.g. as shown in the respective line in Table II, application no. 8, column 5 or 8, by for example in one embodiment expression either in the cytosol or in an organelle such as a plastid or mitochondria or both, preferably in a plastid, or in another embodiment non-targeted or targeted.

The nucleic acid molecule of the invention comprises a nucleotide sequence or molecule which hybridizes, preferably hybridizes under stringent conditions as defined herein, to one of the nucleic acid molecule shown in the respective line in Table I, preferably Table IB, application no. 8, columns 5 or 8, preferably the coding region thereof, fragments or homologs thereof, and encodes a protein having above-mentioned activity, e.g. conferring the production or the increased production of the respective fine chemical linoleic acid as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted, and optionally, the activity thereof is selected from the group consisting of 3-ketoacyl-CoA thiolase, 3-oxoacyl-[acyl-carrier-protein] synthase, AAS bifunctional protein, ABC transporter permease protein, acid shock protein, aconitate hydratase, arginine decarboxylase, aromatic acid decarboxylase, aspartate-semialdehyde dehydrogenase, At1g19800-protein, At2g45420-protein, ATPase epsilon subunit, b0801-protein, b1330-protein, b1470-protein, b1670-protein, b1837-protein, b2032-protein, b2099-protein, b2107-protein, b2399-protein, b2474-protein, b2613-protein, b2682-protein, b2849-protein, b2909-protein, beta-hydroxylase, carbamoyl-phosphate synthase subunit, cation-transporting ATPase, CCAAT-binding transcription factor, chlorophyllase, colanic acid biosynthesis protein, coproporphyrinogen III oxidase, CTP synthetase, DnaJ-like chaperone, electron transport complex protein, ethanolamine utilization protein, fatty acid desaturase, Fe—S subunit of oxidoreductase, flavodoxin, fumarate hydratase, gamma-glutamyltranspeptidase, glucose-6-phosphate 1-dehydrogenase, glutaredoxin, glycin cleavage system protein, GMO2LC38418-protein, heat shock transcription factor, hydrolase, lipoprotein precursor, membrane transport protein, metal-dependent hydrolase, molecular chaperone, monothiol glutaredoxin, multiple antibiotic resistance protein, outer membrane receptor protein precursor, oxidoreductase, phosphate import ATP-binding protein, phosphoribosylformyl glycinamidine synthase subunit, phosphotriesterase, protein kinase, protein phosphatase, pyruvate kinase, recombinase A, sll1032-protein, sn-glycerol-3-phosphate transport system permease protein, ThiF family protein, threonine dehydratase, threonine efflux protein, transcription factor, transcriptional regulator protein, tryptophan synthase alpha chain, TTC0490-protein, xanthine dehydrogenase, ydr507c-protein, and YGR104C-protein, respectively.

Moreover, the nucleic acid molecule of the invention can comprise only a portion of the coding region of one of the nucleic acid molecules depicted in Table I, application no. 8, columns 5 or 8, for example a fragment which can be used as a probe or primer or a fragment encoding a biologically active portion of the polypeptide of the present invention or of a polypeptide used in the process of the present invention, i.e. having above-mentioned activity, e.g. conferring the production or the increased production of the respective fine chemical linoleic acid as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted. The nucleotide sequences determined from the cloning of the present protein-according-to-the-invention-encoding gene allows for the generation of probes and primers designed for use in identifying and/or cloning its homologues from other cell types and organisms. The probe/primer typically comprises a substantially purified oligonucleotide. The oligonucleotide typically comprises a region of nucleotide sequence that hybridizes under stringent conditions to at least about 12, 15 preferably about 20 or 25, more preferably about 40, 50 or 75 consecutive nucleotides of a sense strand of one of the sequences set forth, e.g., in the respective line in Table I, application no. 8, column 5 or 8, preferably the coding region thereof, or a homolog or a fragment thereof, an anti-sense sequence of one of the sequences, e.g., set forth in the respective line in Table I, application no. 8, columns 5 or 8, preferably the coding region thereof, or a homolog or a fragment thereof or naturally occurring mutants thereof. Primers based on a nucleic acid molecule of the invention can be used in PCR reactions to clone homologs of the polypeptide of the invention or of the polypeptide used in the process of the invention, e.g. as the primers described in the examples of the present invention, e.g. as shown in the examples. A PCR with the primers shown in the respective line in Table III, column 8 will result in a fragment of the gene product as shown in Table II, application no. 8, column 5.

Primer sets are interchangeable. The person skilled in the art knows how to design and combine said primers to result in the desired product, e.g. in a full length clone or a partial sequence. Probes based on the sequences of the nucleic acid molecule of the invention or used in the process of the present invention can be used to detect transcripts or genomic sequences encoding the same or homologous proteins. The probe can further comprise a label group attached thereto, e.g. the label group can be a radioisotope, a fluorescent compound, an enzyme, or an enzyme co-factor. Such probes can be used as a part of a genomic marker test kit for identifying cells which express a polypeptide of the invention or used in the process of the present invention, such as by measuring a level of an encoding nucleic acid molecule in a sample of cells, e.g. detecting mRNA levels or determining, whether a genomic gene comprising the sequence of the polynucleotide of the invention or used in the processes of the present invention has been mutated or deleted.

As the expression of a respective nucleic acids of the invention is related to the synthesis of the respective fine chemical linoleic acid its function as a probe extends to the detection of microorganisms, plant tissues, plants, plant variets, plant ecotypes or plant genera with varying capability or potential for synthesis of the respective fine chemical linoleic acid. Therefore in one embodiment the present invention relates to a method for analyzing the capability or potential of a plant tissue, a plant, a plant variety or plant ecotype to produce the fine chemical linoleic acid by using the nucleic acid of the invention or parts thereof as a probe to detect the amount of the nucleic acid of the invention in the non-human organism or a part thereof in comparison to another non-human organism.

The nucleic acid molecule of the invention encodes a polypeptide or portion thereof which includes an amino acid sequence which is sufficiently homologous to the amino acid sequence shown in respective line in Table II, application no. 8, columns 5 or 8 such that the protein or portion thereof maintains the ability to confer the production or the increased production of the respective fine chemical linoleic acid as compared to a corresponding, e.g. non-transformed, wild type non-human organism, like a microorganism or a plant cell, plant or part thereof, in particular increasing the activity as mentioned above or as described in the examples in microorganisms or plants.

As used herein, the language “sufficiently homologous” refers to proteins or portions thereof which have amino acid sequences which include a minimum number of identical or equivalent amino acids (e.g. an amino acid which has a similar side chain as an amino acid in one of the sequences of the polypeptide of the present invention) to an amino acid sequence shown in the respective line in Table II, application no. 8, columns 5 or 8 such that the protein or portion thereof is able to confer the production or the increased production of the respective fine chemical linoleic acid as compared to a corresponding, e.g. non-transformed, wild type non-human organism, like a microorganism or a plant cell, plant or part thereof. For example having the activity of a protein as shown in the respective line in Table II, application no. 8, column 3 and as described herein.

In an embodiment of the present invention, the nucleic acid molecule of the present invention comprises a nucleic acid molecule that encodes a portion of the protein of the present invention. The protein is at least about 30%, 35%, 40%, 45% 50%, 55%, 60%, 65%, 70%, 75%, 80%, 85%, 90%, 91%, 92%, 93%, 94%, 95%, 96%, 97%, 98%, 99%, 99.5% or more homologous to an entire amino acid sequence shown in the respective line of Table II, application no. 8, column 5 or 8 and has the above-mentioned activity, e.g. conferring the production or the increased production of the respective fine chemical linoleic acid as compared to a corresponding, e.g. non-transformed, wild type non-human organism, like a microorganism or a plant cell, plant or part thereof by, for example in a embodiment expression either in the cytosol or in an organelle such as a plastid or mitochondria or both, preferably in a plastid, or in another embodiment by targeted or non-targeted expression.

Portions of proteins encoded by the nucleic acid molecule of the invention show preferably the above-mentioned activity, e.g. conferring the production or the increased production of the fine chemical as compared to a corresponding, e.g. non-transformed, wild type non-human organism, like a microorganism or a plant cell, plant or part thereof.

As mentioned herein, the term “biologically active portion” is intended to include a portion, e.g., a domain/motif, that confers the production or the increased production of the fine chemical as compared to a corresponding, e.g. non-transformed, wild type non-human organism, like a microorganism or a plant cell, plant or part thereof or has an immunological activity such that it binds to an antibody binding specifically to the polypeptide of the present invention or a polypeptide used in the process of the present invention for conferring the production or the increased production of the fine chemical as compared to a corresponding, e.g.

non-transformed, wild type non-human organism, like a microorganism or a plant cell, plant or part thereof.

The invention further relates to nucleic acid molecules or methods using said nucleic acid molecules that differ from one of the nucleotide sequences shown in the respective line in Table I, application no. 8, columns 5 or 8, preferably the coding region thereof, or fragments or homologs thereof, due to degeneracy of the genetic code but encode a polypeptide of the present invention, in particular a polypeptide having the above-mentioned activity, e.g. as those polypeptides depicted in the respective line in Table II, application no. 8, columns 5 or 8, fragments or homologs thereof. Advantageously, the nucleic acid molecule of the invention comprises, or in an other embodiment has, a nucleotide sequence encoding a protein comprising, or in an other embodiment having, an amino acid sequence shown in the respective line in Table II, application no. 8, columns 5 or 8, fragments or homologs thereof. In a further embodiment, the nucleic acid molecule of the invention encodes a full length protein which is substantially homologous to an amino acid sequence shown in the respective line in Table II, application no. 8, columns 5 or 8, or homologs thereof. However, in an embodiment, the nucleic acid molecule of the present invention does not consist of the sequence shown in the respective line in Table I, preferably Table IA, application no. 8, columns 5 or 8, preferably the coding region thereof, but the functional homologs thereof.

In addition, it will be appreciated by those skilled in the art that DNA sequence polymorphisms that lead to changes in the amino acid sequences may exist within a population of the non-human organism of the present invention. Also these non-human organisms are encompassed by the respective non-human organism according to the invention.

As used herein, the terms “gene” and “recombinant gene” refer to nucleic acid molecules comprising an open reading frame encoding the polypeptide of the invention or comprising the nucleic acid molecule of the invention or encoding the polypeptide used in the process of the present invention, preferably from a plant, especially a crop plant, or from a microorganism useful for the method of the invention. Such genetic polymorphism in the gene encoding the polypeptide of the invention or comprising the nucleic acid molecule of the invention may exist among individuals within a population due to natural variation. Such natural variations can typically result in 1 to 5% variance in the nucleotide sequence of the gene. Any and all such nucleotide variations and resulting amino acid polymorphisms in genes encoding a polypeptide of the invention or comprising a the nucleic acid molecule of the invention that are the result of natural variation and that do not alter the functional activity as described are intended to be within the scope of the invention.

Nucleic acid molecules corresponding to natural variants of a nucleic acid molecule of the invention, which can also be a cDNA, can be isolated based on their homology to the nucleic acid molecules disclosed herein using the nucleic acid molecule of the invention, or a portion thereof, as a hybridization probe according to standard hybridization techniques under stringent hybridization conditions.

Accordingly, in another embodiment, a nucleic acid molecule of the invention is at least 15, 20, 25 or 30 nucleotides in length. Preferably, it hybridizes under stringent conditions to a nucleic acid molecule comprising a nucleotide sequence of the nucleic acid molecule of the present invention or used in the process of the present invention, e.g. comprising the sequence shown in the respective line in Table I, application no. 8, columns 5 or 8, preferably the coding region thereof. In another embodiment the nucleic acid molecule is preferably at least 20, 30, 50, 100, 250 or more nucleotides in length.

The term “hybridizes under stringent conditions” is defined above. In an embodiment, the term “hybridizes under stringent conditions” is intended to describe conditions for hybridization and washing under which nucleotide sequences at least 30%, 40%, 50% or 65% identical to each other typically remain hybridized to each other. Preferably, the conditions are such that sequences at least about 70%, 75%, 80%, 85%, 90% or 95% or more identical to each other typically remain hybridized to each other.

Preferably, a nucleic acid molecule of the invention that hybridizes under stringent conditions to a sequence shown in the respective line in Table I, application no. 8, columns 5 or 8, preferably the coding region thereof, corresponds to a naturally-occurring nucleic acid molecule of the invention. As used herein, a “naturally-occurring” nucleic acid molecule refers to a RNA or DNA molecule having a nucleotide sequence that occurs in nature (e.g. encodes a natural protein). Preferably, the nucleic acid molecule encodes a natural protein having above-mentioned activity, e.g. conferring the production or the increased production of the respective fine chemical linoleic acid as compared to a corresponding, e.g. non-transformed, wild type non-human organism, like a microorganism or a plant cell, plant or part thereof after increasing the expression or activity thereof or the activity of a protein of the invention or used in the process of the invention, in an embodiment for example expression either in the cytosol or in an organelle such as a plastid or mitochondria, preferably in plastids, or, in another embodiment by targeted or non-targeted expression.

In addition to naturally-occurring variants of the sequences of the polypeptide or nucleic acid molecule of the invention as well as of the polypeptide or nucleic acid molecule used in the process of the invention that may exist in the population, the skilled artisan will further appreciate that changes can be introduced by mutation into a nucleotide sequence of the nucleic acid molecule of the invention, e.g. encoding the polypeptide of the invention or used in the process of the present invention, thereby leading to changes in the amino acid sequence of the encoded said polypeptide, without altering the functional ability of the polypeptide, preferably not decreasing said activity, preferably increasing said activity.

For example, nucleotide substitutions leading to amino acid substitutions at “non-essential” amino acid residues can be made in a sequence of the nucleic acid molecule of the invention or used in the process of the invention, e.g. shown in the respective line in Table I, application no. 8, columns 5 or 8, preferably the coding region thereof, or fragments or homologs thereof.

With regard to the activity of a polypeptide a “non-essential” amino acid residue is a residue that can be altered without altering the activity of said polypeptide, whereas an “essential” amino acid residue is required for an activity as mentioned above, e.g. conferring the production or the increased production of the fine chemical as compared to a corresponding, e.g. non-transformed, wild type non-human organism, like a microorganism or a plant cell, plant or part thereof. Other amino acid residues, however, (e.g., those that are not conserved or only semi-conserved in the domain having said activity) may not be essential for activity and thus are likely to be amenable to alteration without altering said activity.

Further, a person skilled in the art knows that the codon usage between organisms can differ. In the enclosed sequence potocol according to WIPO ST 25 the respective donor organism codon usage “translation” is used.The person skilled in the art may adapt the codon usage in the nucleic acid molecule of the present invention to the usage of the organism or the cell compartment for example of the plastid or mitochondria in which the polynucleotide or polypeptide is expressed.

Accordingly, the invention relates to nucleic acid molecules encoding a polypeptide having above-mentioned activity, in a non-human organisms or parts thereof by for example expression either in the cytosol or in an organelle such as a plastid or mitochondria or both, preferably in plastids, that contain changes in amino acid residues that are not essential for said activity. Such polypeptides differ in amino acid sequence from a sequence depicted in the respective line in Table II, especially Table IIA, application no. 8, columns 5 or 8, but retain said activity described herein. The nucleic acid molecule can comprise a nucleotide sequence encoding a polypeptide, wherein the polypeptide comprises an amino acid sequence at least about 50% identical to an amino acid sequence shown in the respective line in Table II, application no. 8, columns 5 or 8, and is capable of conferring the production or the increased production of the respective fine chemical linoleic acid as compared to a corresponding, e.g. non-transformed, wild type non-human organism, like a microorganism or a plant cell, plant or part thereof, after increasing its activity for example in an embodiment by expression either in the cytosol or in an organelle such as a plastid or mitochondria or both, preferably in plastids, or, in another embodiment by targeted or non-targeted expression. Preferably, the protein encoded by the nucleic acid molecule is at least about 60%, 70%, 80%, 90%, 95%, 96%, 97%, 98%, 99% or 99.5% identical to the sequence shown in the respective line in Table II, application no. 8, columns 5 or 8.

To determine the percentage identity (=homology, herein used interchangeably) of two amino acid sequences or of two nucleic acid molecules, the sequences are written one underneath the other for an optimal comparison (for example gaps may be inserted into the sequence of a protein or of a nucleic acid in order to generate an optimal alignment with the other protein or the other nucleic acid). The amino acids or nucleobases at the respective corresponding positions are then compared. If a position in one sequence is occupied by the same amino acid or the same nucleobase as the corresponding position in the other sequence, the molecules are homologous at this position. The percentage identity between the two sequences is a function of the number of identical positions shared by the sequences (i.e. % homology=% identity=number of identical positions/total number of positions×100).

For the determination of the percentage identity (=homology) of two or more amino acid sequences or of two or more nucleic acid molecules several computer software programs have been developed. The homology of two or more sequences can be calculated with for example the software Fasta, which presently has been used in the version Fasta 3 (Pearson W. R. and Lipman D. J., PNAS 85, 2444 (1988); Pearson W. R., Methods in Enzymology 183, 63 (1990)). Another useful program for the calculation of homologies of different sequences is the standard blast program, which is included in the Biomax PEDANT™ software (Biomax, Munich, Federal Republic of Germany). This leads unfortunately sometimes to suboptimal results since blast does not always include complete sequences of the subject and the query. Nevertheless as this program is very efficient it can be used for the comparison of a huge number of sequences. The following settings are typically used for such a comparison of sequences: -p Program Name [String]; -d Database [String]; default=nr; -i Query File [File In]; default=stdin; -e Expectation value (E) [Real]; default=10.0; -m alignment view options: 0=pairwise; 1=query-anchored showing identities; 2=query-anchored no identities; 3=flat query-anchored, show identities; 4=flat query-anchored, no identities; 5=query-anchored no identities and blunt ends; 6=flat query-anchored, no identities and blunt ends; 7=XML Blast output; 8=tabular; 9 tabular with comment lines [Integer]; default=0; -o BLAST report Output File [File Out] Optional; default=stdout; -F Filter query sequence (DUST with blastn, SEG with others) [String]; default=T; -G Cost to open a gap (zero invokes default behavior) [Integer]; default=0; -E Cost to extend a gap (zero invokes default behavior) [Integer]; default=0; -X X dropoff value for gapped alignment (in bits) (zero invokes default behavior); blastn 30, megablast 20, tblastx 0, all others 15 [Integer]; default=0; -I Show GI's in deflines [T/F]; default=F; -q Penalty for a nucleotide mismatch (blastn only) [Integer]; default=−3; -r Reward for a nucleotide match (blastn only) [Integer]; default=1; -v Number of database sequences to show one-line descriptions for (V) [Integer]; default=500; -b Number of database sequence to show alignments for (B) [Integer]; default=250; -f Threshold for extending hits, default if zero; blastp 11, blastn 0, blastx 12, tblastn 13; tblastx 13, megablast 0 [Integer]; default=0; -g Perfom gapped alignment (not available with tblastx) [T/F]; default=T; -Q Query Genetic code to use [Integer]; default=1; -D DB Genetic code (for tblast[nx] only) [Integer]; default=1; -a Number of processors to use [Integer]; default=1; -O SeqAlign file [File Out] Optional; -J Believe the query defline [T/F]; default=F; -M Matrix [String]; default=BLOSUM62; -W Word size, default if zero (blastn 11, megablast 28, all others 3) [Integer]; default=0; -z Effective length of the database (use zero for the real size) [Real]; default=0; -K Number of best hits from a region to keep (off by default, if used a value of 100 is recommended) [Integer]; default=0; -P 0 for multiple hit, 1 for single hit [Integer]; default=0; -Y Effective length of the search space (use zero for the real size) [Real]; default=0; -S Query strands to search against database (for blast[nx], and tblastx); 3 is both, 1 is top, 2 is bottom [Integer]; default=3; -T Produce HTML output [T/F]; default=F; -I Restrict search of database to list of GI's [String] Optional; -U Use lower case filtering of FASTA sequence [T/F] Optional; default=F; -y X dropoff value for ungapped extensions in bits (0.0 invokes default behavior); blastn 20, megablast 10, all others 7 [Real]; default=0.0; -Z X dropoff value for final gapped alignment in bits (0.0 invokes default behavior); blastn/megablast 50, tblastx 0, all others 25 [Integer]; default=0; -R PSI-TBLASTN checkpoint file [File In] Optional; -n MegaBlast search [T/F]; default=F; -L Location on query sequence [String] Optional; -A Multiple Hits window size, default if zero (blastn/megablast 0, all others 40 [Integer]; default=0; -w Frame shift penalty (OOF algorithm for blastx) [Integer]; default=0; -t Length of the largest intron allowed in tblastn for linking HSPs (0 disables linking) [Integer]; default=O.

Results of high quality are reached by using the algorithm of Needleman and Wunsch or Smith and Waterman. Therefore programs based on said algorithms are preferred. Advantageously the comparisons of sequences can be done with the program PileUp (J. Mol. Evolution., 25, 351 (1987), Higgins et al., CABIOS 5, 151 (1989)) or preferably with the programs “Gap” and “Needle”, which are both based on the algorithms of Needleman and Wunsch (J. Mol. Biol. 48, 443 (1970)), and “BestFit”, which is based on the algorithm of Smith and Waterman (Adv. Appl. Math. 2, 482 (1981)). “Gap” and “BestFit” are part of the GCG softwarepackage (Genetics Computer Group, 575 Science Drive, Madison, Wis., USA 53711 (1991); Altschul et al., (Nucleic Acids Res. 25, 3389 (1997)), “Needle” is part of the The European Molecular Biology Open Software Suite (EMBOSS) (Trends in Genetics 16 (6), 276 (2000)). Therefore preferably the calculations to determine the percentages of sequence homology are done with the programs “Gap” or “Needle” over the whole range of the sequences. The following standard adjustments for the comparison of nucleic acid sequences were used for “Needle”: matrix: EDNAFULL, Gap_penalty: 10.0, Extend_penalty: 0.5. The following standard adjustments for the comparison of nucleic acid sequences were used for “Gap”: gap weight: 50, length weight: 3, average match: 10.000, average mismatch: 0.000.

For example a sequence, which has 80% homology with sequence SEQ ID NO: 1061 at the nucleic acid level is understood as meaning a sequence which, upon comparison with the sequence SEQ ID NO: 1061 by the above-mentioned program “Needle” with the above-mentioned parameter set, has a 80% homology.

Homology between two polypeptides is understood as meaning the identity of the amino acid sequence over in each case the entire sequence length which is calculated by comparison with the aid of the above program “Needle” using Matrix: EBLOSUM62, Gap_penalty: 8.0, Extend_penalty: 2.0.

For example a sequence which has a 80% homology with sequence SEQ ID NO: 1062 at the protein level is understood as meaning a sequence which, upon comparison with the sequence SEQ ID NO: 1062 by the above-mentioned program “Needle” with the abovementioned parameter set, has a 80% homology.

Functional equivalents of the nucleic acid molecules according to the present invention and/or used in the process according to the present invention represent an embodiment of homologs. Functional equivalents derived from the nucleic acid molecules as shown in the respective line in Table I, application no. 8, columns 5 or 8, preferably the coding region thereof, according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the nucleic acid molecules as shown in the respective line in Table I, application no. 8, columns 5 or 8, preferably the coding region thereof, according to the invention and encode polypeptides having essentially the same properties as the polypeptide as shown in the respective line in Table II, application no. 8, columns 5 or 8.

Functional equivalents derived from one of the polypeptides as shown in the respective line in Table II, application no. 8, columns 5 or 8 according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the polypeptides as shown in the respective line in Table II, application no. 8, columns 5 or 8 according to the invention and having essentially the same properties as the polypeptide as shown in the respective line in Table II, application no. 8, columns 5 or 8.

“Essentially the same properties” of a functional equivalent is above all understood as meaning that the functional equivalent has the above-mentioned activity, by for example in an embodiment expression either in the cytosol or in an organelle such as a plastid or mitochondria or both, or, in another embodiment by targeted or non-targeted expression, while increasing the amount of protein, activity or function of said functional equivalent in an organism, e.g. a microorganism, a plant or plant tissue or animal tissue, plant or animal cells or a part of the same.

A nucleic acid molecule encoding a homolog to a polypeptide depicted in the respective line in Table II, application no. 8, columns 5 or 8 can be created by introducing one or more nucleotide substitutions, additions or deletions into the nucleic acid molecule of the present invention, in particular of Table I, application no. 8, columns 5 or 8, in the respective line, such that one or more amino acid substitutions, additions or deletions are introduced into the encoded protein. Mutations can be introduced into the nucleic acid molecules as depicted in the respective line in Table I, application no. 8, columns 5 or 8, preferably the coding region thereof, by standard techniques, such as site-directed mutagenesis, PCR-mediated mutagenesis or other methods known to the person skilled in the art.

Preferably, conservative amino acid substitutions are made at one or more predicted non-essential amino acid. A “conservative amino acid substitution” is one in which the amino acid is replaced with an amino acid having a similar side chain. Families of amino acids having similar side chains have been defined in the art. These families include amino acids with basic side chains (e.g. lysine, arginine, histidine), acidic side chains (e.g. aspartic acid, glutamic acid), uncharged polar side chains (e.g. glycine, asparagine, glutamine, serine, threonine, tyrosine, cysteine), nonpolar side chains (e.g. alanine, valine, leucine, isoleucine, proline, phenylalanine, methionine, tryptophane), beta-branched side chains (e.g. threonine, valine, isoleucine) and aromatic side chains (e.g. tyrosine, phenylalanine, tryptophane, histidine).

Thus, a predicted non-essential amino acid in a polypeptide of the invention or a polypeptide used in the process of the invention is preferably replaced with another amino acid from the same family. Alternatively, in another embodiment, mutations can be introduced randomly along all or part of a coding sequence of a nucleic acid molecule of the invention or used in the process of the invention, such as by saturation mutagenesis, and the resultant mutants can be screened for activity described herein to identify mutants that retain or even have increased activity, e.g. conferring the production or the increased production of the fine chemical as compared to a corresponding, e.g. non-transformed, wild type non-human organism, like a microorganism or a plant cell, plant or part thereof.

Following mutagenesis of one of the sequences as shown herein, the encoded protein can be expressed recombinantly and the activity of the protein can be determined using, for example, assays described herein (see Examples) or by measuring the resulting fine chemical production in an organism expressing the mutagenized form of the sequence in comparison to an organism expressing the original non-mutagenized form of the sequence.

Homologues of the nucleic acid sequences used, with the sequences shown in the respective line in Table I, application no. 8, columns 5 or 8, preferably the coding region thereof, comprise also allelic variants with at least approximately 30%, 35%, 40%, 45%, 50%, 55%, 60%, 65%, 70%, 75%, 80%, 85%, 90%, 91%, 92%, 93%, 94% 95%. 96%, 97%, 98%, 99%, 99.5% or more % homology with one of the nucleotide sequences shown or the abovementioned derived nucleic acid sequences or their homologues or derivatives or parts of these. Allelic variants encompass in particular functional variants which can be obtained by deletion, insertion or substitution of nucleotides from the sequences shown in the respective line, preferably in Table I, columns 5 or 8, preferably the coding region thereof, or from the derived nucleic acid sequences, the intention being, however, that the enzymatic activity or the biological activity of the resulting proteins synthesized is advantageously retained or increased.

In one embodiment of the present invention, the nucleic acid molecule of the invention or used in the process of the invention comprises the sequences shown in the respective line in any of the Table I, application no. 8, columns 5 or 8, preferably the coding region thereof, fragments or homologs thereof. It is preferred that the nucleic acid molecule comprises as little as possible other nucleotides not shown in the respective line in any one of Table I, application no. 8, columns 5 or 8, preferably the coding region thereof, fragments or homologs thereof. In one embodiment, the nucleic acid molecule comprises less than 500, 400, 300, 200, 100, 90, 80, 70, 60, 50 or 40 further nucleotides. In a further embodiment, the nucleic acid molecule comprises less than 30, 20 or 10 further nucleotides. In one embodiment, the nucleic acid molecule used in the process of the invention is identical to the sequences shown in the respective line in Table I, application no. 8, columns 5 or 8, preferably the coding region thereof.

Also preferred is that the nucleic acid molecule used in the process of the invention encodes a polypeptide comprising the sequence shown in the respective line in Table II, application no. 8, columns 5 or 8, fragments or homologs thereof. In one embodiment, the nucleic acid molecule encodes less than 150, 130, 100, 80, 60, 50, 40 or 30 further amino acids. In a further embodiment, the encoded polypeptide comprises less than 20, 15, 10, 9, 8, 7, 6 or 5 further amino acids. In one embodiment used in the inventive process, the encoded polypeptide is identical to the sequences shown in the respective line in Table II, application no. 8, columns 5 or 8.

In one embodiment, the nucleic acid molecule of the invention or used in the process encodes a polypeptide comprising the sequence shown in the respective line in Table II, application no. 8, columns 5 or 8, or fragments or homologs thereof, and comprises less than 100 further nucleotides. In a further embodiment, said nucleic acid molecule comprises less than 30 further nucleotides. In one embodiment, the nucleic acid molecule used in the process is identical to a coding sequence of the sequences shown in the respective line in Table I, application no. 8, columns 5 or 8.

Polypeptides (=proteins), which still have the essential biological or enzymatic activity of the polypeptide of the present invention conferring the production or the increased production of the respective fine chemical linoleic acid as compared to a corresponding, e.g. non-transformed, wild type non-human organism, like a microorganism or a plant cell, plant or part thereof, i.e. whose activity is essentially not reduced, are polypeptides with at least 10%, 20%, 30%, 40%, 50%, 60%, 70%, 80%, 90%, 95% or more of the wild type biological activity or enzymatic activity, advantageously, the activity is essentially not reduced in comparison with the activity of a polypeptide shown in the respective line in Table II, application no. 8, columns 5 or 8 expressed under identical conditions.

Homologues of nucleic acid molecules shown in the respective line in Table I, application no. 8, columns 5 or 8, preferably the coding region thereof, or fragments thereof, or of the derived sequences shown in the respective line in Table II, application no. 8, columns 5 or 8, or fragments thereof, also mean truncated sequences, cDNA, single-stranded DNA or RNA of the coding and noncoding DNA sequence. Homologues of said sequences are also understood as meaning derivatives, which comprise noncoding regions such as, for example, UTRs, introns, terminators, enhancers or promoter variants. The promoters upstream of the nucleotide sequences stated can be modified by one or more nucleotide substitution(s), insertion(s) and/or deletion(s) without, however, interfering with the functionality or activity either of the promoters, the open reading frame (=ORF) or with the 3′-regulatory region such as terminators or other 3′-regulatory regions, which are far away from the ORF. It is furthermore possible that the activity of the promoters is increased by modification of their sequence, or that they are replaced completely by more active promoters, even promoters from heterologous organisms. Appropriate promoters are known to the person skilled in the art and are mentioned herein below.

Accordingly, one embodiment of the invention relates to a host cell, which has been transformed stably or transiently with the vector according to the invention or the nucleic acid molecule according to the invention or the nucleic acid construct or the expression construct according to the invention.

Depending on the non-human host organism, the non-human organisms used in the process according to the invention are cultured or grown in a manner with which the skilled worker is familiar.

As a rule, microorganisms are grown in a liquid medium comprising a carbon source, usually in the form of sugars, a nitrogen source, usually in the form of organic nitrogen sources such as yeast extract or salts such as ammonium sulfate, trace elements such as iron salts, manganese salts, magnesium salts, and, if appropriate, vitamins, at temperatures between 0° C. and 100° C., preferably between 10° C. and 60° C., while passing in oxygen. In the event the microorganism is anaerobe, no oxygen is blown through the culture medium. The pH value of the liquid nutrient medium may be kept constant, that is to say regulated during the culturing phase, or not. The organisms may be cultured batch wise, semi batch wise or continuously. Nutrients may be provided at the beginning of the fermentation or fed in semi continuously or continuously. Advantageously microorganisms such as algae are grown under sunlight in open ponds or in fermentors illuminated with a light intensity between 10 to 2000 μmol×m⁻²×s⁻¹, preferred between 100 to 1000 μmol×m⁻²×s⁻¹, more preferred between 200 to 800 μmol×m^{-b 2}×s⁻¹, most preferred between 300 to 600 μmol×m⁻²×s⁻¹. The cells are grown between several hours for example 3 to 48 h and several days 1 to 20 days, preferably 2 to 10 days. Algae as autotrophic organisms grow well in the presence of light as energy source, inorganic hydrogen donors and CO₂ as sole carbon source.

As a rule, plants used in the process according to the invention are grown according to general knowledge of a skilled worker.

The fine chemical produced can be isolated from the non-human organism by methods with which the skilled worker is familiar. For example via extraction, salt precipitation or chromatography, like ion-exchange chromatography. To this end, the non-human organisms, especially the microorganism or the plant, may be disrupted beforehand. The process according to the invention can be conducted batch wise, semi batch wise or continuously. A summary of known culture and isolation techniques can be found in the textbook by Chmiel (“Bioprozeβtechnik 1, Einührung in die Bioverfahrenstechnik”, Gustav Fischer Verlag, Stuttgart, 1991)), Demain et al. (“Industrial Microbiology and Biotechnology”, 2^{nd} edition, ASM Press, Washington, D.C., 1999, ISBN 1-55581-128-0) or in the textbook by Storhas (“Bioreaktoren and periphere Einrichtungen”, Vieweg Verlag, Braunschweig/Wiesbaden, 1994)).

In one embodiment, the present invention relates to a polypeptide encoded by the nucleic acid molecule according to the present invention, preferably conferring the production of or an increase in the fine chemical content in an non-human organism or cell after generating or increasing the expression or activity of said nucleic acid molecule, either in the cytosol or in an organelles such as a plastid or mitochondrion or both, preferably in a plastid, or in another embodiment by targeted or non-targeted expression.

The present invention also relates to a process for the production of a polypeptide according to the present invention, the polypeptide being expressed in a host cell according to the invention, preferably in a microorganism or a plant cell, plant or a part thereof, especially in a transgenic microorganism or a transgenic plant cell, plant or a part thereof.

In an embodiment, the nucleic acid molecule used in the process for the production of the polypeptide (FCRP) is derived from a microorganism such as a eukaryotic or prokaryotic microorganism, preferably from a eukaryotic microorganism, such as an algae or Saccharomyces cerevisiae; in an embodiment thereof this polypeptide is produced in another microorganism or in another embodiment thereof this polypeptide is produced in a plant cell, plant or a part thereof. In another embodiment, the nucleic acid molecule used in the process for the production of the polypeptide is derived from a plant, in an embodiment thereof the polypeptide is produced in a microorganism or in another embodiment thereof this polypeptide is produced in another plant, a part or cell thereof.

The skilled worker knows that protein and DNA expressed in different non-human organisms differ in many respects and properties, e. g. DNA modulation and imprinting, such as methylation or post-translational modification, as for example glucosylation, phosphorylation, acetylation, myristoylation, ADP-ribosylation, farnesylation, carboxylation, sulfation, ubiquination, etc. though having the same coding sequence. Preferably, the cellular expression control of the corresponding protein differs accordingly in the control mechanisms controlling the activity and expression of an endogenous protein or another prokaryotic or eukaryotic protein. One major difference between proteins expressed in prokaryotic or eukaryotic organisms is the amount and pattern of glycosylation. For example in E. coli there are no glycosylated proteins.

Proteins expressed in yeasts have a high mannose content in the glycosylated proteins, whereas in plants the glycosylation pattern is complex.

The polypeptide (FCRP) of the present invention is preferably produced by recombinant DNA techniques. For example, a nucleic acid molecule encoding the protein is cloned into a vector (as described above), the vector is introduced into a host cell (as described above) and said polypeptide is expressed in the host cell. Said polypeptide (FCRP) can then be isolated from the cells by an appropriate purification scheme using standard protein purification techniques. Alternative to recombinant expression, the polypeptide or peptide of the present invention can be synthesized chemically using standard peptide synthesis techniques.

Moreover, a native polypeptide conferring the increase of the respective fine chemical linoleic acid in a non-human organism or a part thereof can be isolated from cells (e.g., endothelial cells), for example using the antibody of the present invention as described below, in particular, an antibody against proteins having 3-ketoacyl-CoA thiolase, 3-oxoacyl-[acyl-carrier-protein] synthase, AAS bifunctional protein, ABC transporter permease protein, acid shock protein, aconitate hydratase, arginine decarboxylase, aromatic acid decarboxylase, aspartate-semialdehyde dehydrogenase, At1g19800-protein, At2g45420-protein, ATPase epsilon subunit, b0801-protein, b1330-protein, b1470-protein, b1670-protein, b1837-protein, b2032-protein, b2099-protein, b2107-protein, b2399-protein, b2474-protein, b2613-protein, b2682-protein, b2849-protein, b2909-protein, beta-hydroxylase, carbamoyl-phosphate synthase subunit, cation-transporting ATPase, CCAAT-binding transcription factor, chlorophyllase, colanic acid biosynthesis protein, coproporphyrinogen III oxidase, CTP synthetase, DnaJ-like chaperone, electron transport complex protein, ethanolamine utilization protein, fatty acid desaturase, Fe—S subunit of oxidoreductase, flavodoxin, fumarate hydratase, gammaglutamyltranspeptidase, glucose-6-phosphate 1-dehydrogenase, glutaredoxin, glycin cleavage system protein, GMO2LC38418-protein, heat shock transcription factor, hydrolase, lipoprotein precursor, membrane transport protein, metal-dependent hydrolase, molecular chaperone, monothiol glutaredoxin, multiple antibiotic resistance protein, outer membrane receptor protein precursor, oxidoreductase, phosphate import ATP-binding protein, phosphoribosylformyl glycinamidine synthase subunit, phosphotriesterase, protein kinase, protein phosphatase, pyruvate kinase, recombinase A, sll1032-protein, sn-glycerol-3-phosphate transport system permease protein, ThiF family protein, threonine dehydratase, threonine efflux protein, transcription factor, transcriptional regulator protein, tryptophan synthase alpha chain, TTC0490-protein, xanthine dehydrogenase, ydr507c-protein, or YGR104C-protein activity, respectively, or an antibody against polypeptides as shown in the respective line in Table II, application no. 8, columns 5 or 8, or fragments or homologs thereof which can be produced by standard techniques utilizing the polypeptid of the present invention or fragment thereof, i.e., the polypeptide of this invention (FCRP). Preferred are monoclonal antibodies.

In an embodiment, the present invention relates to a polypeptide encoded by a nucleic acid molecule of the invention or obtainable by a process of the invention. Said polypeptide confers preferably the aforementioned activity, in particular, the polypeptide confers the generation or the increased production of the fine chemical in a cell or an organism or a part thereof after generating or increasing the cellular activity, e.g. by generating or increasing the expression or the specific activity of the polypeptide.

In one embodiment, the present invention relates to a polypeptide as depicted in the respective line in Table II, especially in Table IIA or especially in Table IIB, application no. 8, columns 5 or 8, or fragment or homolog thereof or as coded by the nucleic acid molecule shown in the respective line in Table I, especially Table IA or especially Table IB, application no. 8, columns 5 or 8, preferably the coding region thereof, or fragment or homolog thereof.

In an embodiment, the present invention relates to a polypeptide comprising or consisting of a consensus sequence shown in the respective line in Table IV, application no. 8, column 8 or at least one polypeptide motif shown in the respective line in Table IV, application no. 8, column 8.

In an embodiment thereof, the present invention relates to a polypeptide comprising or consisting of a consensus sequence shown in the respective line in Table IV, application no. 8, column 8 or comprising at least one polypeptide motif shown in the respective line in Table IV, application no. 8, column 8, whereby not more than 20, 15, 10, 9, 8, 7, 6, 5, 4, 3, 2, or 1, or 0 of the amino acids can be replaced by any amino acid.

In another embodiment thereof, the present invention relates to a polypeptide comprising or consisting of a consensus sequence shown in the respective line in Table IV, application no. 8, column 8 or comprising at least one polypeptide motif shown in the respective line in Table IV, application no. 8, column 8, whereby not more than 15%, 10%, 5%, 4%, 3%, 2% or1% or 0% of the amino acids defined as distinct specific amino acids are/is replaced by another amino acid.

In another embodiment thereof, the present invention relates to a polypeptide comprising or consisting of a consensus sequence shown in the respective line in Table IV, application no. 8, column 8 or comprising at least one polypeptide motif shown in the respective line in Table IV, application no. 8, column 8, whereby not more than 20, 15, 10, 9, 8, 7, 6, 5, 4, 3, 2, or 1, or 0 amino acids are inserted into the consensus sequence or polypeptide motif.

In an embodiment of the present invention, the polypeptide of the present invention comprises or consists of a consensus sequence or at least one polypeptide motif shown in the respective line in Table IV, application no. 8, column 8, whereby said polypeptide distinguishes over the sequence depicted in the respective line in Table II, application no. 8, columns 5 or 8 by one or more amino acids. In one embodiment, said polypeptide of the invention distinguishes over the sequence shown in the respective line in Table II, application no. 8, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 8, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in the respective line in Table II, application no. 8, columns 5 and 8.

In an embodiment, the invention relates to a polypeptide conferring an increase in the respective fine chemical linoleic acid in a non-human organism, especially a microorganism or a plant, or a part thereof, being encoded by the nucleic acid molecule of the invention or used in the process of the invention and having a sequence which distinguishes over the sequence as shown in the respective line in Table II, application no. 8, columns 5 or 8 by one or more amino acids (but not exceeding 5%, preferably 4%, 3%, 2%, 1%, 0.5%, 0.2% or 0.1%). In an embodiment, said polypeptide of the invention does not comprise or consist of the sequence shown in the respective line in Table II, application no. 8, columns 5 or 8. In an embodiment, said polypeptide of the present invention is less than 100%, 99.999%, 99.99%, 99.9% or 99% identical. In one embodiment, said polypeptide which differs at least in one or more amino acids (but not exceeding 5%, preferably 4%, 3%, 2%, 1%, 0.5%, 0.2% or 0.1%) from the polypeptide shown in the respective line in Table II, application no. 8, columns 5 and 8 does not comprise a protein of the sequence shown in the respective line in Table II A and/or II B, application no. 8, columns 5 or 8.

In an embodiment, the present invention relates to a polypeptide having the activity of the protein as shown in the respective line in Table II, application no. 8, column 3, which distinguishes over the sequence depicted in the respective line in Table II, application no. 8, columns 5 or 8 by one or more amino acids, preferably by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 8, columns 5 and 8 by not more than 80%, 70%, 60%, 50%, 40%, 30%, 20%, 10%, 5%, 4%, 3%, 2%, 1% or 0.5%.

In another embodiment the polypeptide of the invention takes the form of a preprotein consisting of a plastidial or mitochondrial transit peptide joint to a polypeptide having the activity of the protein as shown in Table II, column 3, from which the transit peptide is preferably cleaved off upon transport of the preprotein into the organelle for example into the plastid or mitochondrion.

In another embodiment the polypeptide of the invention takes not the form of a preprotein.

The terms “protein” and “polypeptide” used in this application are interchangeable. “Polypeptide” refers to a polymer of amino acids (amino acid sequence) and does not refer to a specific length of the molecule. Thus peptides and oligopeptides are included within the definition of polypeptide. This term does also refer to or include post-translational modifications of the polypeptide, for example, glycosylations, acetylations, phosphorylations and the like. Included within the definition are, for example, polypeptides containing one or more analogs of an amino acid (including, for example, unnatural amino acids, etc.), polypeptides with substituted linkages, as well as other modifications known in the art, both naturally occurring and nonnaturally occurring.

Another aspect of the present invention pertains to isolated FCRP, or biologically active portions thereof. An “isolated” or “purified” protein or biologically active portion thereof is substantially free of cellular material when produced by recombinant DNA techniques or chemical precursors or other chemicals when chemically synthesized.

The language “substantially free of cellular material” includes preparations of the polypeptide of the invention in which the protein is separated from cellular components of the cells in which it is naturally or recombinantly produced. In an embodiment, the language “substantially free of cellular material” includes preparations having less than about 30% (by dry weight) of “contaminating protein”, more preferably less than about 20% of “contaminating protein”, still more preferably less than about 10% of “contaminating protein”, and most preferably less than about 5% “contaminating protein”. The term “contaminating protein” relates to polypeptides, which differ from the polypeptide in question of the present invention. When the polypeptide of the present invention or biologically active portion thereof is recombinantly produced, it is also preferably substantially free of culture medium, i.e., culture medium represents less than about 20%, more preferably less than about 10%, and most preferably less than about 5% of the volume of the protein preparation. The language “substantially free of chemical precursors or other chemicals” includes preparations in which the polypeptide of the present invention is separated from chemical precursors or other chemicals, which are involved in the synthesis of the protein. The language “substantially free of chemical precursors or other chemicals” includes preparations having less than about 30% (by dry weight) of chemical precursors, other chemicals or other proteins, which differ from the polypeptide in question. Other chemical precursors, other chemicals or other proteins, which are not identical to the proteins as shown in the respective line in Table II, column 5 or 8, or fragments or homologs thereof, are all collectively named as impurities. The term “chemical precursors” shall mean in the sense of the specification chemical substances, which are intermediates of the biochemical pathway within the organism or within the cell(s) of the non-human organism for example glucose-6-phoshat, citrate, fumarate, homoserine etc. The term “other chemicals” shall mean in the sense of the specification chemical substances, which are end products of the biochemical pathway within the organism or within the cell(s) of the organism for example amino acids such as lysine, alanine etc; fatty acids such as linolenic acid, eicosapantaenoic acid etc, sugars such as glucose, mannose, ribose, desoxy ribose etc, vitamins such as vitamin C, vitamin B2 etc. and all other chemical substances of the cell. The term “other proteins” shall mean in the sense of the specification all other proteins, which are not identical to the proteins mentioned in the respective line in Table II, columns 5 or 8, or fragments or homologs thereof. The fine chemical preparations advantageously shall have less than about 25% impurities, preferably less than about 20% impurities, still more preferably less than about 10% impurities, and most preferably less than about 5% impurities. In preferred embodiments, isolated proteins or biologically active portions thereof lack contaminating proteins from the same organism from which the polypeptide of the present invention is derived. Typically, such proteins are produced by recombinant techniques.

A polypeptide of the invention can participate in the process of the present invention. The polypeptide or a portion thereof comprises preferably an amino acid sequence, which is sufficiently homologous to an amino acid sequence shown in the respective line in Table II A and/or II B, application no. 8, columns 5 or 8.

Further, the polypeptide can have an amino acid sequence which is encoded by a nucleotide sequence which hybridizes, preferably hybridizes under stringent conditions as described above, to a nucleotide sequence of the nucleic acid molecule of the present invention. Accordingly, the polypeptide has an amino acid sequence which is encoded by a nucleotide sequence that is at least about 35%, 40%, 45%, 50%, 55%, 60%, 65%, 70%, 75%, 80%, 85%, 90%, 91%, 92%, 93%, 94%, 95%, 96%, 97%, 98%, 99%, 99.5% or more homologous to one of the amino acid sequences as shown in the respective line in Table II A and/or II B, application no. 8, columns 5 or 8, or fragments thereof. The preferred polypeptide of the present invention preferably possesses at least one of the activities according to the invention and described herein. A preferred polypeptide of the present invention includes an amino acid sequence encoded by a nucleotide sequence which hybridizes, preferably hybridizes under stringent conditions, to a nucleotide sequence shown in the respective line in Table I A and/or I B, application no. 8, columns 5 or 8, preferably the coding region thereof, or fragments thereto or which is homologous thereto, as defined above.

The polypeptide of the present invention can vary from the sequences shown in the respective line in Table II A and/or II B, application no. 8, columns 5 or 8 in amino acid sequence due to natural variation or mutagenesis, as described in detail herein. Accordingly, the polypeptide comprise an amino acid sequence which is at least about 35%, 40%, 45%, 50%, 55%, 60%, 65%, 70%, 75%, 80%, 85% or 90, 91%, 92%, 93%, 94%, 95%, 96%, 97%, 98%, 99%, 99.5% or more homologous to an entire amino acid sequence shown in the respective line in Table II A and/or II B, application no. 8, columns 5 or 8.

Biologically active portions of a polypeptide of the present invention include peptides comprising amino acid sequences derived from the amino acid sequence of the polypeptide of the present invention or used in the process of the present invention, e.g., the amino acid sequence shown in the respective line in Table II, application no. 8, columns 5 or 8 or the amino acid sequence of a protein homologous thereto, which include fewer amino acids than a full length polypeptide of the present invention or used in the process of the present invention or the full length protein which is homologous to a polypeptide of the present invention or used in the process of the present invention depicted herein, and exhibit at least one activity of a polypeptide of the present invention or used in the process of the present invention.

Typically, biologically (or immunologically) active portions of i.e. peptides are, for example, 5, 10, 15, 20, 30, 35, 36, 37, 38, 39, 40, 50, 75, 100, 125, 150 or more amino acids in length; especially they comprise a domain or motif with at least one activity or epitope of a polypeptide of the present invention or used in the process of the present invention. Moreover, other biologically active portions, in which other regions of the polypeptide are deleted, can be prepared by recombinant techniques and evaluated for one or more of the activities described herein.

Manipulation of the nucleic acid molecule of the invention may result in the production of a protein having differences from the protein as shown in the respective line in Table II, application no. 8, column 5 or 8. Differences shall mean at least one amino acid different from the sequences as shown in the respective line in Table II, application no. 8, column 5 or 8, preferably at least 2, 3, 4, 5, 6, 7, 8, 9 or 10 amino acids (especially but not exceeding 0.1%, preferably 0.2%, 0.5%, 1%, 2%, 3% 4% or 5%), more preferably at least 15, 20, 25, 30, 35, 40, 45 or 50 amino acids (especially but not exceeding 0.5%, preferably 1%, 2%, 3% 4%, 5%, 10% or 25%) different from the sequences as shown in the respective line in Table II, application no. 8, column 5 or 8. These proteins may be improved in efficiency or activity, or may be more stable and therefore present in greater numbers in the cell as compared to the wild-type cell.

Any mutagenesis strategies for the polypeptide of the present invention or the polypeptide used in the process of the present invention to result in increasing said activity are not meant to be limiting; variations on these strategies will be readily apparent to one skilled in the art. Using such strategies, and incorporating the mechanisms disclosed herein, the nucleic acid molecule and polypeptide of the invention may be utilized to generate algae, ciliates, fungi, other microorganisms like C. glutamicum or plants or parts thereof, expressing proteins as shown in the respective line in Table II, application no. 8, column 5 or 8, or mutated proteins thereof. The nucleic acid molecules and polypeptide molecules of the invention are expressed such that the yield, production, and/or efficiency of production of a desired compound is improved.

This desired compound may be any natural product of the respective non-human organism, especially a microorganism or a plant, which includes the final products of biosynthesis pathways and intermediates of naturally-occurring metabolic pathways, as well as molecules which do not naturally occur in the metabolism of said cells of said non-human organism, but which are produced by said cells of the invention.

The invention also provides chimeric or fusion proteins.

As used herein, a “chimeric protein” or “fusion protein” comprises a polypeptide of the present invention operatively linked to a polypeptide whereby the latter does not confer on its own above-mentioned activity, in particular, which does not confer an increase of content of the fine chemical in a cell or a non-human organism or a part thereof, if its activity is increased.

In one embodiment, a protein (=polypeptide) as shown in the respective line in Table II, application no. 8, column 5 or 8 refers to a polypeptide having an amino acid sequence corresponding to the polypeptide of the invention, especially to a polypeptide as shown in Table II, application no. 8, columns 5 or 8 or homologs thereof, or used in the process of the invention, whereas a “non-inventive protein or polypeptide” or “other polypeptide” refers to a polypeptide having an amino acid sequence corresponding to a protein which is not substantially homologous to a polypeptide of the invention, preferably which is not substantially homologous to a polypeptide or protein as shown in the respective line in Table II, application no. 8, column 5 or 8 or homologs thereof, e.g., a protein which does not confer the activity described herein and which is derived from the same or a different organism.

Within the fusion protein, the term “operatively linked” is intended to indicate that the polypeptide of the invention or a polypeptide used in the process of the invention and the “other polypeptide” or a part thereof are fused to each other so that both sequences fulfill the proposed function addicted to the sequence used. The “other polypeptide” can be fused to the N-terminus or C-terminus preferable to the C-terminus of the polypeptide of the invention or used in the process of the invention. For example, in one embodiment the fusion protein is a GST-LMRP fusion protein in which the sequences of the polypeptide of the invention or the polypeptide used in the process of the invention are fused to the C-terminus of the GST sequences. Such fusion proteins can facilitate the purification of recombinant polypeptides of the invention or a polypeptide useful in the process of the invention.

In another preferred embodiment, the fusion protein is a polypeptide of the invention or a polypeptide used in the process of the invention containing a heterologous signal sequence, preferably at its N-terminus. In certain host cells (e.g., mammalian host cells), expression and/or secretion of a polypeptide of the invention or a polypeptide used in the process of the invention can be increased through use of a heterologous signal sequence. As already mentioned above, targeting sequences, are required for targeting the gene product into specific cell compartment (for a review, see Kermode, Crit. Rev. Plant Sci. 15 (4), 285 (1996) and references cited therein), for example into the vacuole, the nucleus, all types of plastids, such as amyloplasts, chloroplasts, chromoplasts, the extracellular space, the mitochondria, the endoplasmic reticulum, elaioplasts, peroxisomes, glycosomes, and other compartments of cells or extracellular. Sequences, which must be mentioned in this context, are, in particular, the signalpeptide-or transit-peptide-sequences which are known by the person skilled in the art. For example, plastid or mitochondrial-transit-peptide- sequences enable the targeting of the expression product into the plastids or the mitochondria, respectively, of a plant cell or the mitochondria of a microorganism. Targeting sequences are especially known for eukaryotic and to a lower extent for prokaryotic organisms and can advantageously be operable linked with the nucleic acid molecule of the present invention to achieve an expression in one of said compartments or extracellular.

Preferably, a chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. The fusion gene can be synthesized by conventional techniques including automated DNA synthesizers. Alternatively, PCR amplification of gene fragments can be carried out using anchor primers, which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons (1992)). Moreover, many expression vectors are commercially available that already encode a fusion moiety (e.g., a GST polypeptide). The nucleic acid molecule of the invention can be cloned into such an expression vector such that the fusion moiety is linked in-frame to the encoded protein.

Furthermore, folding simulations and computer redesign of structural motifs of the protein of the invention can be performed using appropriate computer programs (Olszewski, Proteins 25, 286 (1996); Hoffman, Comput. Appl. Biosci. 11, 675 (1995)). Computer modeling of protein folding can be used for the conformational and energetic analysis of detailed peptide and protein models (Monge, J. Mol. Biol. 247, 995 (1995); Renouf, Adv. Exp. Med. Biol. 376, 37 (1995)). The appropriate programs can be used for the identification of interactive sites the polypeptide of the invention or polypeptides used in the process of the invention and its substrates or binding factors or other interacting proteins by computer assistant searches for complementary peptide sequences (Fassina, Immunomethods 114 (1994)). This can be used to identify motifs and domains of the polypeptides according to the present invention or used in the process of the present invention. Further appropriate computer systems for the design of protein and peptides are described in the prior art, for example in Berry, Biochem. Soc. Trans. 22, 1033 (1994); Wodak, Ann. N.Y. Acad. Sci. 501, 1 (1987); Pabo, Biochemistry 25, 5987 (1986). The results obtained from the above-described computer analysis can be used for, e.g., the preparation of peptidomimetics of the protein of the invention or fragments thereof. Such pseudopeptide analogues of the natural amino acid sequence of the protein may very efficiently mimic the parent protein (Benkirane, J. Biol. Chem. 271, 33218 (1996)). For example, the replacement of Gly-Gly in a protein of the present invention or a fragment thereof by the easily available achiral omega-amino acid delta-amino valeric acid results in the substitution of —CH2CONHCH₂—by —CH₂CH₂CH₂CH₂—, thereby providing a replacement of an amido group by an ethylen group, and a convenient strategy for constructing a peptidomimetic (Banerjee, Biopolymers 39, 769 (1996)).

Furthermore, a three-dimensional and/or crystallographic structure of the protein of the invention or used in the process of the present invention and the identification of motifs and/or interactive sites of the polypeptide of the invention or used in the process of the present invention and its substrates or binding factors can be used for the design of mutants with modulated binding or turnover activities. For example, the active center of the polypeptide of the present invention can be modeled and amino acids participating in the catalytic reaction can be modulated to increase or decrease the binding of the substrate to activate or improve the polypeptide. The identification of the active center, respective motifs and the amino acids involved in the catalytic reaction facilitates the screening for mutants having an increased activity.

Another embodiment of the invention relates to an antibody, which binds specifically to the polypeptide according to the invention or a portion thereof, i.e. specific fragments or epitopes of such a polypeptide.

The antibodies of the invention can be used to identify and isolate the polypeptide according to the invention in any organism, preferably microorganism or plants. These antibodies can be monoclonal antibodies, polyclonal antibodies or synthetic antibodies as well as fragments of antibodies, such as Fab, Fv or scFv fragments etc. Antibodies can be made by many well-known methods (see, e.g. Harlow and Lane, “Antibodies; A Laboratory Manual”, Cold Spring Harbor Laboratory, Cold Spring Harbor, N.Y., (1988)). Briefly, purified antigen can be injected into an animal in an amount and in intervals sufficient to elicit an immune response. Antibodies can either be purified directly, or spleen cells can be obtained from the animal. The cells can then be fused with an immortal cell line and screened for antibody secretion. The antibodies can be used to screen nucleic acid clone libraries for cells secreting the antigen. Those positive clones can then be sequenced. See, for example, Kelly et al., Bio/Technology 10, 163 (1992); Bebbington et al., Bio/Technology 10, 169 (1992).

As the nucleic acid molecules of the invention and/or the expression thereof is related to the synthesis of the respective fine chemical linoleic acid its function as a probe extends to the detection of microorganisms, plant tissues, plants, plant variets, plant ecotypes or plant genera with varying, advantageously increased, capability or potential for synthesis of the respective fine chemical linoleic acid. Therefore in one embodiment the present invention relates to a method for analyzing the capability or potential of a plant tissue, a plant, a plant variety or ecotype to produce the respective fine chemical linoleic acid by using the respective antibody of the invention as a probe to detect the amount of the polypeptide encoded by said nucleic acid molecule of the invention in a non-human organism in comparison to another organism.

Therefore in one embodiment the invention relates to a method to probe a non-human organism for its capability for the production of the respective fine chemical comprising the steps of
a) analyzing the sequence and/or the expression of the respective nucleic acid molecule of the invention in least two non-human organisms which differ in the capability to produce the respective fine chemical;
b) associate the different sequence and/or expression level of the nucleic acid molecules of the invention with their different capability to produce the respective fine chemical;
c) analyze the sequence and/or the expression of the nucleic acid molecule of the invention in at least a third non-human organism;
d) predict the capability of the at least third organism to produce the respective fine chemical based by comparing the sequence and/or expression of the nucleic acid of the invention to the sequences and/or expression levels of the nucleic acid molecule of the invention of the at least two organisms analyzed in step a.

The phrases “selectively binds” and “specifically binds” with the polypeptide refer to a binding reaction that is determinative of the presence of the polypeptide in a heterogeneous population of polypeptides and other biologics. Thus, under designated immunoassay conditions, the specified antibodies bound to a particular polypeptide but do not bind in a significant amount to other polypeptides present in the sample. Selective binding of an antibody under such conditions may require an antibody that is selected for its specificity for a particular polypeptide. A variety of immunoassay formats may be used to select antibodies that selectively bind with a particular polypeptide. For example, solid-phase ELISA immunoassays are routinely used to select antibodies selectively immunoreactive with a polypeptide. See Harlow and Lane,

“Antibodies, A Laboratory Manual,” Cold Spring Harbor Publications, New York, (1988), for a description of immunoassay formats and conditions that could be used to determine selective binding.

In some instances, it is desirable to prepare monoclonal antibodies from various hosts. A description of techniques for preparing such monoclonal antibodies may be found in Stites et al., eds., “Basic and Clinical Immunology,” (Lange Medical Publications, Los Altos, Calif., Fourth Edition) and references cited therein, and in Harlow and Lane, “Antibodies, A Laboratory Manual,” Cold Spring Harbor Publications, New York (1988).

Gene expression in non-human organism, like microorganism or plants, especially plants, is regulated by the interaction of protein transcription factors with specific nucleotide sequences within the regulatory region of a gene. One example of transcription factors are polypeptides that contain zinc finger (ZF) motifs. Each ZF module is approximately 30 amino acids long and folds around a zinc ion. The DNA recognition domain of a ZF protein is an ahelical structure that inserts into the major groove of the DNA double helix. The module contains three amino acids that bind to the DNA with each amino acid contacting a single base pair in the target DNA sequence. ZF motifs are arranged in a modular repeating fashion to form a set of fingers that recognize a contiguous DNA sequence. For example, a three-fingered ZF motif will recognize 9 by of DNA. Hundreds of proteins have been shown to contain ZF motifs with between 2 and 37 ZF modules in each protein (Isalan M. et al., Biochemistry 37 (35), 12026 (1998); Moore M. et al., Proc. Natl. Acad. Sci. USA 98 (4), 1432 (2001) and Moore M. et al., Proc. Natl. Acad. Sci. USA 98 (4), 1437 (2001); U.S. Pat Nos. 6,007,988 and 6,013,453).

The regulatory region of a gene contains many short DNA sequences (cisacting elements) that serve as recognition domains for transcription factors, including ZF proteins. Similar recognition domains in different genes allow the coordinate expression of several genes encoding enzymes in a metabolic pathway by common transcription factors. Variation in the recognition domains among members of a gene family facilitates differences in gene expression within the same gene family, for example, among tissues and stages of development and in response to environmental conditions.

Typical ZF proteins contain not only a DNA recognition domain but also a functional domain that enables the ZF protein to activate or repress transcription of a specific gene. Experimentally, an activation domain has been used to activate transcription of the target gene (U.S. Pat No. 5,789,538 and patent application WO 95/19431), but it is also possible to link a transcription repressor domain to the ZF and thereby inhibit transcription (WO 00/47754 and WO 01/002019). It has been reported that an enzymatic function such as nucleic acid cleavage can be linked to the ZF (WO 00/20622).

The invention provides a method that allows one skilled in the art to isolate the regulatory region of one or more FCRP encoding genes from the genome of a non-human organism, preferably a plant cell and to design zinc finger transcription factors linked to a functional domain that will interact with the regulatory region of the gene. The interaction of the zinc finger protein with the gene can be designed in such a manner as to alter expression of the gene and preferably thereby to confer the production or the increased production of the fine chemical as compared to a corresponding, e.g. non-transformed, wild type non-human organism, like a microorganism or a plant cell, plant or part thereof.

A further embodiment of the invention also relates to a method for the generation of a transgenic host or host cell, e.g. a eukaryotic or prokaryotic cell, preferably a transgenic microorganism, a transgenic plant cell or a transgenic plant tissue or a transgenic plant, which comprises introducing, into the plant cell, the plant or a part thereof , the nucleic acid molecule according to the invention, the nucleic acid construct according to the invention or the vector according to the invention or the expression cassette according to the invention.

A further embodiment of the invention also relates to a method for the stable generation of a host or host cell, prokaryotic or eukaryotic cell, preferably a transgenic microorganism such as a transgenic algae, a transgenic plant cell or a transgenic plant or a transgenic part thereof, which comprises introducing, into the plant cell, the plant or a part thereof, the nucleic acid construct according to the invention, the vector according to the invention, the expression cassette according to the invention, or the nucleic acid molecule according to the invention, whereby the introduction of the nucleic acid molecule, nucleic acid construct, expression cassette and/or vector is in such manner that the transformants are stable during the propagation of the host in respect of the introduced nucleic acid molecules, nucleic acid construct, expression cassette and/or vector.

A further embodiment of the invention also relates to a method for the transient generation of a host or host cell, prokaryotic or eukaryotic cell, preferably a transgenic microorganism such as a transgenic algae, a transgenic plant cell or a transgenic plant or a transgenic part thereof, which comprises introducing, into the plant cell, the plant or a part thereof, the nucleic acid construct according to the invention, the vector according to the invention, the expression cassette according to the invention or the nucleic acid molecule according to the invention, whereby the introduced nucleic acid molecule, nucleic acid construct, expression cassette and/or vector is not integrated into the genome of the host or host cell. Therefore the transformants are not stable during the propagation of the host in respect of the introduced nucleic acid molecules, nucleic acid construct, expressions cassette and/or vector.

In the process according to the invention, transgenic non-human organisms are also to be understood as meaning—if they take the form of plants—plant cells, plant tissues, plant organs such as root, shoot, stem, seed, flower, tuber or leaf, or intact plants which are grown for the production of the fine chemical.

Growing is to be understood as meaning for example culturing the transgenic plant cells, plant tissue or plant organs on or in a nutrient medium or the intact plant or part thereof on or in a substrate, for example in hydroponic culture, potting compost or on a field soil.

In a further advantageous embodiment of the process, the nucleic acid molecules can be expressed in single-celled plant cells (such as algae), see Falciatore et al., Marine Biotechnology 1 (3), 239 (1999), and references cited therein, and plant cells from higher plants (for example spermatophytes such as crops). Examples of plant expression vectors encompass those which are described in detail herein or in D. Becker, Plant Mol. Biol. 20,1195 (1992), M. W Bevan, [Nucl. Acids Res. 12, 8711(1984), and in “Vectors for Gene Transfer in Higher Plants” in: Transgenic Plants, Vol. 1, Engineering and Utilization, eds. S. D. Kung and R. Wu, Academic Press, 1993, pp. 15-38]. An overview of binary vectors and their use is also found in R. Hellens, Trends in Plant Science,5 (10), 446 (2000).

Vector DNA can be introduced into prokaryotic or eukaryotic cells via conventional transformation or transfection techniques. The terms “transformation” and “transfection” include conjugation and transduction and, as used in the present context, are intended to encompass a multiplicity of prior-art methods for introducing foreign nucleic acid molecules (for example DNA) into a host cell, including calcium phosphate coprecipitation or calcium chloride coprecipitation, DEAE-dextran-mediated transfection, PEG-mediated transfection, lipofection, natural competence, chemically mediated transfer, electroporation or particle bombardment. Suitable methods for the transformation or transfection of host cells, including plant cells, can be found in Sambrook et al. (Molecular Cloning: A Laboratory Manual., 2^{nd} Ed., Cold Spring Harbor Laboratory, Cold Spring Harbor Laboratory Press, Cold Spring Harbor, N.Y., 1989) and in other laboratory handbooks such as Methods in Molecular Biology, 1995, Vol. 44, Agrobacterium protocols, eds. Gartland and Davey, Humana Press, Totowa, N.J.

The above-described methods for the transformation and, if desired, regeneration of plants from plant cells, plant tissues or parts of a plant are exploited for transient or stable transformation of plants. Suitable methods are the transformation of protoplasts by polyethylene-glycol-induced DNA uptake, the biolistic method with the gene gun—known as the particle bombardment method—electroporation, the incubation of dry embryos in DNA-containing solution, microinjection and the Agrobacterium-mediated gene transfer. The above-mentioned methods are described for example in B. Jenes, Techniques for Gene Transfer, in: Transgenic Plants, Vol. 1, Engineering and Utilization, edited by Kung S. D. and Wu R., Academic Press, pp. 128-143 (1993) and in Potrykus, Annu. Rev. Plant Physiol. Plant Molec. Biol. 42, 205 (1991). The construct to be expressed is preferably cloned into a vector, which is suitable for transforming Agrobacterium tumefaciens, for example pBin19 (Bevan, Nucl. Acids Res. 12, 8711 (1984)). Agrobacteria transformed with such a vector can then be used in the known manner for the transformation of plants, in particular crop plants, such as, for example, tobacco plants, for example by bathing scarified leaves or leaf segments in an agrobacterial solution and subsequently culturing them in suitable media. The transformation of plants with Agrobacterium tumefaciens is described for example by Hofgen and Willmitzer in Nucl. Acid Res. 16, 9877 (1988) or known from, inter alia, White F.F., “Vectors for Gene Transfer in Higher Plants” in Transgenic Plants, Vol. 1, Engineering and Utilization, edited by Kung S. D. and Wu R., Academic Press, 1993, pp. 15-38. Alternatively the construct to be expressed can be cloned into vectors suitable for plastid transformation, as for example described in WO 2004/029256, WO 20040/04445 or Dufourmantel et al., Plant Mol. Biol. 55, 479 (2004).

To select for the successful transfer of the nucleic acid molecule, vector or nucleic acid construct of the invention according to the invention into a host organism, it is advantageous to use marker genes as have already been described above in detail. It is known of the stable or transient integration of nucleic acids into plant cells that only a minority of the cells takes up the foreign DNA and, if desired, integrates it into its genome, depending on the expression vector used and the transfection technique used. To identify and select these integrants, a gene encoding for a selectable marker (as described above, for example resistance to antibiotics) is usually introduced into the host cells together with the gene of interest. Preferred selectable markers in plants comprise those, which confer resistance to an herbicide such as glyphosate or gluphosinate. Other suitable markers are, for example, markers, which encode genes involved in biosynthetic pathways of, for example, sugars or amino acids, such as β-galactosidase, ura3 or ilv2. Markers, which encode genes such as luciferase, gfp or other fluorescence genes, are likewise suitable. These markers and the aforementioned markers can be used in mutants in which these genes are not functional since, for example, they have been deleted by conventional methods. Furthermore, nucleic acid molecules, which encode a selectable marker, can be introduced into a host cell on the same vector as those, which encode the polypeptides of the invention or used in the process or else in a separate vector. Cells which have been transfected stably with the nucleic acid introduced can be identified for example by selection (for example, cells which have integrated the selectable marker survive whereas the other cells die).

Since the marker genes, as a rule specifically the gene for resistance to antibiotics and herbicides, are no longer required or are undesired in the transgenic host cell once the nucleic acids have been introduced successfully, the process according to the invention for introducing the nucleic acids advantageously employs techniques which enable the removal, or excision, of these marker genes. One such a method is what is known as cotransformation. The cotransformation method employs two vectors simultaneously for the transformation, one vector bearing the nucleic acid according to the invention and a second bearing the marker gene(s). A large proportion of transformants receives or, in the case of plants, comprises (up to 40% of the transformants and above), both vectors. In case of transformation with Agrobacteria, the transformants usually receive only a part of the vector, the sequence flanked by the T-DNA, which usually represents the expression cassette. The marker genes can subsequently be removed from the transformed plant by performing crosses. In another method, marker genes integrated into a transposon are used for the transformation together with desired nucleic acid (known as the Ac/Ds technology). The transformants can be crossed with a transposase resource or the transformants are transformed with a nucleic acid construct conferring expression of a transposase, transiently or stable. In some cases (approx. 10%), the transposon jumps out of the genome of the host cell once transformation has taken place successfully and is lost. In a further number of cases, the transposon jumps to a different location. In these cases, the marker gene must be eliminated by performing crosses. In microbiology, techniques were developed which make possible, or facilitate, the detection of such events. A further advantageous method relies on what are known as recombination systems, whose advantage is that elimination by crossing can be dispensed with. The best-known system of this type is what is known as the Cre/lox system. Crel is a recombinase, which removes the sequences located between the loxP sequences. If the marker gene is integrated between the loxP sequences, it is removed, once transformation has taken place successfully, by expression of the recombinase. Further recombination systems are the HI N/H IX, FLP/FRT and REP/STB system (Tribble et al., J. Biol. Chem., 275, 22255 (2000); Velmurugan et al., J. Cell Biol., 149, 553 (2000)). A site-specific integration into the plant genome of the nucleic acid sequences according to the invention is possible.

Naturally, these methods can also be applied to microorganisms such as yeast, fungi or bacteria. Also methods for the production of marker-free plastid transformants using a transiently cointegrated selection gene have been described for example by Koop et al., Nature Biotechology, 22 (2), 225 (2004).

Agrobacteria transformed with an expression vector according to the invention may also be used in the manner known per se for the transformation of plants such as experimental plants like Arabidopsis or crop plants, such as, for example, cereals, maize, oats, rye, barley, wheat, soya, rice, cotton, sugarbeet, canola, sunflower, flax, hemp, potato, tobacco, tomato, carrot, bell peppers, oilseed rape, tapioca, cassava, arrow root, tagetes, alfalfa, lettuce and the various tree, nut, and grapevine species, in particular oil-containing crop plants such as soya, peanut, castor-oil plant, sunflower, maize, cotton, flax, oilseed rape, coconut, oil palm, safflower (Carthamus tinctorius) or cocoa beans, or in particular crop plants, like cereals, maize, oats, rye, barley, wheat, soya, rice, cotton, sugarbeet, canola, sunflower, potato or oilseed rape, for example by bathing scarified leaves or leaf segments in an agrobacterial solution and subsequently growing them in suitable media.

In addition to the transformation of somatic cells, which then have to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic (Feldman K. A. and Marks M. D., Mol. Gen. Genet. 208, 274 (1987), Feldmann K . in: Koncz C., Chua N.-H. and Shell J., eds., “Methods in Arabidopsis Research”, Word Scientific, Singapore, (1992) pp. 274-289). Alternative methods are based on the repeated removal of the influorescences and incubation of the excision site in the center of the rosette with transformed agrobacteria, whereby transformed seeds can likewise be obtained at a later point in time (Chang, Plant J., 5, 551 (1994), Katavic, Mol. Gen. Genet. 245, 363 (1994)). However, an especially effective method is the vacuum infiltration method with its modifications such as the “floral dip” method. In the case of vacuum infiltration of Arabidopsis, intact plants under reduced pressure are treated with an agrobacterial suspension (Bechthold N., C. R. Acad. Sci. Paris Life Sci, 316, 1194 (1993)), while in the case of the” floral dip” method the developing floral tissue is incubated briefly with a surfactanttreated agrobacterial suspension (Clough S. J., and Bent A. F., Plant J. 16, 735 (1998)). A certain proportion of transgenic seeds are harvested in both cases, and these seeds can be distinguished from non-transgenic seeds by growing under the above-described selective conditions. In addition the stable transformation of plastids is of advantages because plastids are inherited maternally in most crops reducing or eliminating the risk of transgene flow through pollen. The transformation of the chloroplast genome is generally achieved by a process, which has been schematically displayed in Klaus et al., (Nature Biotechnology 22 (2), 225 (2004)). Briefly the sequences to be transformed are cloned together with a selectable marker gene between flanking sequences homologous to the chloroplast genome. These homologous flanking sequences direct site specific integration into the plastome. Plastidal transformation has been described for many different plant species and an overview can be taken from Bock R., J. Mol. Biol. 312 (3), 425 (2001) or Maliga P., Trends Biotechnol. 21, 20 (2003). Further biotechnological progress has recently been reported in form of marker free plastid transformants, which can be produced by a transient cointegrated maker gene (Klaus et al., Nature Biotechnology 22 (2), 225 (2004)).

The genetically modified plant cells can be regenerated via all methods with which the skilled worker is familiar. Suitable methods can be found in the above-mentioned publications by Kung S. D. and Wu R., Potrykus or Hofgen and Willmitzer.

Accordingly, the present invention thus also relates to a plant cell comprising the nucleic acid construct according to the invention, the expression cassette according to the invention, the nucleic acid molecule according to the invention or the vector according to the invention, as well as a process to generate such a plant cell.

Accordingly the present invention relates to any cell transgenic for any nucleic acid characterized as part of the invention, e.g. conferring the increase of the respective fine chemical linoleic acid in a cell or a non-human organism or a part thereof, e.g. the nucleic acid molecule of the invention, the nucleic acid construct of the invention, the vector of the invention, the expression cassette according to the invention, or a nucleic acid molecule encoding the polypeptide of the invention, e.g. encoding a polypeptide having an activity as the protein as shown in the respective line in Table II, application no. 8, column 3. Due to the abovementioned activity the respective fine chemical linoleic acid content in a cell or a non-human organism is increased. For example, due to modulation or manipulation, the cellular activity is increased, in a preferred embodiment in organelles such as plastids or mitochondria, e.g. due to an increased expression or specific activity or specific targeting of the subject matters of the invention in a cell or a non-human organism or a part thereof especially in organelles such as plastids or mitochondria, or in another embodiment in the cytosol. Transgenic for a polypeptide having a protein or a protein activity means herein that due to modulation or manipulation of the genome, the activity of protein as shown in the respective line in Table II, application no. 8, column 3 or a protein as shown in the respective line in Table II, application no. 8, column 3-like activity is increased in the cell or non-human organism or part thereof, especially in organelles such as plastids or mitochondria, or especially in the cytosol. Examples are described above in context with the process of the invention.

“Transgenic”, for example regarding a nucleic acid molecule, a nucleic acid construct, an expression cassette or a vector comprising said nucleic acid molecule or a non-human organism transformed with said nucleic acid molecule, nucleic acid construct, expression cassette or vector, refers to all those subjects originating by recombinant methods in which either
a) the nucleic acid sequence, or
b) a genetic control sequence linked operably to the nucleic acid sequence, for example a promoter, or
c) (a) and (b);

are not located in their natural genetic environment or have been modified by recombinant methods, an example of a modification being a substitution, addition, deletion, inversion or insertion of one or more nucleotide residues. Natural genetic environment refers to the natural chromosomal locus in the organism of origin, or to the presence in a genomic library. In the case of a genomic library, the natural genetic environment of the nucleic acid sequence is preferably retained, at least in part. The environment flanks the nucleic acid sequence at least at one side and has a sequence of at least 50 bp, preferably at least 500 bp, especially preferably at least 1000 bp, very especially preferably at least 5000 bp, in length.

A naturally occurring expression cassette—for example the naturally occurring combination of the promoter of the gene encoding a protein as shown in the respective line in Table II, application no. 8, column 5 or 8, preferably the coding region thereof, or fragments or homologs thereof with the corresponding encoding gene—becomes a transgenic expression cassette when it is modified by non-natural, synthetic “artificial” methods such as, for example, mutagenesis. Such methods have been described (U.S. Pat. No. 5,565,350; WO 00/15815; also see above).

Further, the plant cell, plant or a part thereof can also be transformed such that further enzymes and proteins are (over)expressed which expression supports an increase of the fine chemical.

However, transgenic also means that the nucleic acids according to the invention are located at their natural position in the genome of a non-human organism, but that the sequence has been modified in comparison with the natural sequence and/or that the regulatory sequences of the natural sequences have been modified. Preferably, transgenic/recombinant is to be understood as meaning the transcription of the nucleic acids used in the process according to the invention occurs at a non-natural position in the genome, that is to say the expression of the nucleic acids is homologous or, preferably, heterologous. This expression can be performed with transiently transformed organism or with stably transformed organism.

Transgenic plants comprising the respective fine chemical(s) synthesized in the process according to the invention can be marketed directly without isolation of the fine chemical synthesized. In the process according to the invention, plants are understood as meaning all plant parts, plant organs such as leaf, stalk, root, tubers or seeds or propagation material or harvested material or the intact plant. In this context, the seed encompasses all parts of the seed such as the seed coats, epidermal cells, seed cells, endosperm or embryonic tissue. The fine chemical produced in the process according to the invention may, however, also be isolated from the plant. In case of linoleic acid this can be in free form or bound to proteins. Fine chemical(s) produced by this process can be harvested by harvesting the non-human organisms either from the culture in which they grow or from the field. For example, this can be done via squeezing, grinding and/or extraction, salt precipitation and/or ion-exchange chromatography of the plant parts, preferably the plant seeds, plant fruits, plant tubers and the like.

In a further embodiment, the present invention relates to a process for the generation of a microorganism, comprising the introduction, into the microorganism or parts thereof, of the nucleic acid construct of the invention, or the expression cassette of the invention, or the vector of the invention or the nucleic acid molecule of the invention.

In another embodiment, the present invention relates also to a transgenic microorganism comprising the nucleic acid molecule of the invention, the nucleic acid construct of the invention, or the expression cassette of the invention, or the vector of the invention. Appropriate microorganisms have been described herein before under source organism, preferred are in particular aforementioned strains suitable for the production of fine chemicals.

In principle all microorganisms can be used as host organism especially the ones mentioned under source organism above. It is advantageous to use in the process of the invention transgenic microorganisms such as algae selected from the group of the families Bacillariophyceae, Charophyceae, Chlorophyceae, Chrysophyceae, Craspedophyceae, Euglenophyceae, Prymnesiophyceae, Phaeophyceae, Dinophyceae, Rhodophyceae, Xanthophyceae, Prasinophyceae and its described species and strains. Examples for such algae are the following species Isochrysis galbana, Chaetoceros gracilis, Chaetoceros calcitrans, Tetraselmis suecica, Thalassiosira pseudonana, Pavlova lutheri, lsochrysis sp., Skeletonema costatum, Chroomonas salina, Dunaliella tertiolecta, Chaetoceros simplex, Chaetoceros muelleri, Nannochloropsis sp., Cyclotella sp., Phaeodactylum tricornutum, Tetraselmis chui, Pavlova salina, Dicruteria sp., Tetraselmis levis, Dunaliella perva, Thalassiosira weissfloggii, Chlamydomonas sp., Chlorella vulgaris, Neochloris oleoabundans or Chlorella sp, which are only small overview.

The process of the invention is, when the host organisms are microorganisms, advantageously carried out at a temperature between 0° C. and 95° C., preferably between 10° C. and 85° C., particularly preferably between 15° C. and 75° C., very particularly preferably between 15° C. and 45° C. The pH is advantageously kept at between pH 4 and 12, preferably between pH 6 and 9, particularly preferably between pH 7 and 8, during this. The process of the invention can be operated batch wise, semi batch wise or continuously. A summary of known cultivation methods is to be found in the textbook by Chmiel (Bioprozeβtechnik 1. Einführung in die Bioverfahrenstechnik (Gustav Fischer Verlag, Stuttgart, 1991)) or in the textbook by Storhas (Bioreaktoren and periphere Einrichtungen (Vieweg Verlag, Braunschweig/Wiesbaden, 1994)). The culture medium to be used must meet the requirements of the respective strains in a suitable manner. Descriptions of culture media for various microorganisms are present in the handbook “Manual of Methods for General Bacteriology” of the American Society for Bacteriology (Washington D.C., USA, 1981) and for algae in McLellan et al. (“Maintenance of algae and protozoa”, in “Maintenance of Microorganisms”, eds. Doyle A. and Kirsop B., London pp. 183-208 (1991)), Provasoli et al. (“Artificial media for freshwater algae: problems and suggestions”, in “The Ecology of Algae”, eds. Hartman R. T., Pymatunig Laboratory of Field Biology Special publication 2, University of Pittsburgh, pp. 84-96 (1960)) or Starr R. C. (“Algal cultures-sources and methods of cultivation”, in “Photosynthesis Part A, Methods in Enzymology 23”, eds. San Pietro A., N. Y., pp 29-53 (1971)). These media, which can be employed according to the invention include, as described above, usually one or more carbon sources, nitrogen sources, inorganic salts, vitamins and/or trace elements. Preferred carbon sources are sugars such as mono-, di- or polysaccharides. Examples of very good carbon sources are glucose, fructose, mannose, galactose, ribose, sorbose, ribulose, lactose, maltose, sucrose, raffinose, starch or cellulose. Sugars can also be added to the media via complex compounds such as molasses, or other byproducts of sugar refining. It may also be advantageous to add mixtures of various carbon sources. Other possible carbon sources are oils and fats such as, for example, soybean oil, sunflower oil, peanut oil and/or coconut fat, fatty acids such as, for example, palmitic acid, stearic acid and/or linoleic acid, alcohols and/or polyalcohols such as, for example, glycerol, methanol and/or ethanol and/or organic acids such as, for example, acetic acid and/or lactic acid. Nitrogen sources are usually organic or inorganic nitrogen compounds or materials, which contain these compounds. Examples of nitrogen sources include ammonia in liquid or gaseous form or ammonium salts such as ammonium sulfate, ammonium chloride, ammonium phosphate, ammonium carbonate or ammonium nitrate, nitrates, urea, amino acids or complex nitrogen sources such as corn steep liquor, soybean meal, soybean protein, yeast extract, meat extract and others. The nitrogen sources may be used singly or as a mixture. Inorganic salt compounds, which may be present in the media include the chloride, phosphorus or sulfate salts of calcium, magnesium, sodium, cobalt, molybdenum, potassium, manganese, zinc, copper and iron. For the cultivation of algae the so called soilwater media are preferred. Such media are composed of soil extract, trace element solutions, filtered seawater, a nitrogen source and a buffer substance. Such culture media are well known by the skilled person and are available for example from culture collections such as the culture collection of algae (SAG) at the University of Gottingen, the Culture collection of algae in Coimbra, Portugal (ACOI) or the culture collection of algae (UTEX) in Texas, USA.

It is possible to use as phosphorus source phosphoric acid, potassium dihydrogenphosphate or dipotassium hydrogenphosphate or the corresponding sodium-containing salts. Chelating agents can be added to the medium in order to keep the metal ions in solution. Particularly suitable chelating agents include dihydroxyphenols such as catechol or protocatechuate, or organic acids such as citric acid. The fermentation media employed according to the invention for cultivating microorganisms normally also contain other growth factors such as vitamins or growth promoters, which include, for example, biotin, riboflavin, thiamine, folic acid, nicotinic acid, pantothenate and pyridoxine. All media components are sterilized either by heat (1.5 bar and 121° C. for 20 min) or by sterilizing filtration. The components can be sterilized either together or, if necessary, separately. All media components can be present at the start of the cultivation or optionally be added continuously or batch wise. The temperature of the culture is normally between 0° C. and 55° C., preferably at 10° C. to 30° C., and can be kept constant or changed during the experiment. The pH of the medium should be in the range from 3,5 to 8.5, preferably in the range between 5 to 7. The pH for the cultivation can be controlled during the cultivation by adding basic compounds such as sodium hydroxide, potassium hydroxide, ammonia or aqueous ammonia or acidic compounds such as phosphoric acid or sulfuric acid. Foaming can be controlled by employing antifoams such as, for example, fatty acid polyglycol esters. In addition the stability of plasmids can be maintained by adding to the medium suitable substances having a selective effect, for example antibiotics, if the plasmids carry the genetic information to overcome the selective effect. Aerobic conditions are maintained by introducing oxygen or oxygen-containing gas mixtures such as, for example, ambient air into the culture. The temperature of the culture is normally from 20° C. to 45° C. and preferably from 25° C. to 40° C. The culture is continued until formation of the desired product is at a maximum. This aim is normally achieved within 10 hours to 160 hours.

The fermentation broths obtained in this way, normally have a dry matter content of 7.5 to 25% by weight. The fermentation broth can be processed further. Depending on requirements, the biomass can be removed entirely or partly by separation methods, such as, for example, centrifugation, filtration, decantation or a combination of these methods, from the fermentation broth or left completely in it. The fermentation broth can then be thickened or concentrated by known methods, such as, for example, with the aid of a rotary evaporator, thin-film evaporator, falling film evaporator, by reverse osmosis or by nanofiltration. This concentrated fermentation broth can then be worked up by freeze-drying, spray drying, spray granulation or by other processes.

In another embodiment, the present invention relates to a method of producing a transgenic non-human organism, especially a microorganism or a plant, with a FCRP coding nucleic acid, wherein expression of the nucleic acid(s) in the microorganism or a plant confers the production or the increased production of the fine chemical as compared to a wild type non-human organism comprising: (a) transforming a non-human cell with a vector comprising a FCRP encoding nucleic acid, and (b) generating from the non-human cell a transgenic non-human organism with production or enhanced production of the fine chemical.

The present invention relates also to a process according to the present invention whereby the produced fine chemical composition or the produced the fine chemical is isolated.

In this manner, more than 50% by weight, advantageously more than 60% by weight, preferably more than 70% by weight, especially preferably more than 80% by weight, very especially preferably more than 90% by weight, of the fine chemical produced in the process can be isolated. The resulting fine chemical can, if appropriate, subsequently be further purifled, if desired mixed with other active ingredients such as vitamins, amino acids, carbohydrates, antibiotics and the like, and, if appropriate, formulated.

However, it is also possible to purify the fine chemical produced further with methods known by a person skilled in the art, like extraction, precipitation, crystallization etc. For this purpose, the product-containing composition is for example subjected to a chromatography on a suitable resin, in which case the desired product or the impurities are retained wholly or partly on the chromatography resin. These chromatography steps can be repeated if necessary, using the same or different chromatography resins. The skilled worker is familiar with the choice of suitable chromatography resins and their most effective use. The purified product can be concentrated by filtration or ultrafiltration and stored at a temperature at which the stability of the product is a maximum.

The identity and purity of the isolated compound(s) can be determined by prior art techniques. These include gas chromatography (GC), high performance liquid chromatography (HPLC), spectroscopic methods, mass spectrometry (MS), staining methods, thin-layer chromatography, NIRS, enzyme assay or microbiological assays. These analytical methods are summarized for example in: Patek et al., Appl. Environ. Microbiol. 60, 133 (1994), Malakhova et al., Biotekhnologiya 11, 27 (1996); Schmidt et al., Bioprocess Engineer. 19, 67 (1998), Ullmann's Encyclopedia of Industrial Chemistry (1996) Vol. A27, VCH, Weinheim, pp. 89-90, pp. 521-540, pp. 540-547, pp. 559-566, 575-581 and pp. 581-587, Michal G., “Biochemical Pathways: An Atlas of Biochemistry and Molecular Biology”, John Wiley and Sons (1999); Fallon A. et al. “Applications of HPLC in Biochemistry” in “Laboratory Techniques in Biochemistry and Molecular Biology”, Vol. 17 (1987).

The fine chemical obtained in the process are suitable as starting material for the synthesis of further products of value. For example, in case of fatty acids they can be used in combination with each other or alone for the production of pharmaceuticals, foodstuffs, animal feeds or cosmetics.

Accordingly, the present invention relates a method for the production of a pharmaceuticals, food stuff, animal feeds, nutrients or cosmetics comprising the steps of the process according to the invention, including the isolation of the fine chemical produced or a composition produced comprising the fine chemical, and, if desired, formulating the product with a pharmaceutical acceptable carrier or formulating the product in a form acceptable for an application in agriculture or formulating the product in a form acceptable for an application as food stuff, animal feed, nutrient or cosmetic. A further embodiment according to the invention is the use of the fine chemical produced in the process or of the transgenic non-human organisms in animal feeds, foodstuffs, medicines, food supplements, cosmetics or pharmaceuticals.

In yet another aspect, the invention also relates to harvestable parts and to propagation material of the transgenic plants according to the invention which either contain transgenic plant cells expressing a nucleic acid molecule according to the invention or which contains cells which show an increased cellular activity of the polypeptide of the invention, e.g. an increased expression level or higher activity of the described protein.

Harvestable parts can be in principle any useful parts of a plant, for example, flowers, pollen, seedlings, tubers, leaves, stems, fruit, seeds, roots etc. Propagation material includes, for example, seeds, fruits, cuttings, seedlings, tubers, rootstocks etc. Preferred are seeds, fruits, seedlings or tubers as harvestable or propagation material.

The invention furthermore relates to the use of the transgenic non-human organisms according to the invention and of the cells, cell cultures, parts—such as, for example, roots, leaves and the like as mentioned above in the case of transgenic plant organisms—derived from them, and to transgenic propagation material such as seeds or fruits and the like as mentioned above, for the production of foodstuffs or feeding stuffs, pharmaceuticals or fine chemicals.

Further in another embodiment, the present invention relates to the use of the nucleic acid molecule of the invention or used in the method of the invention alone or in combination with other genes of the respective fine chemical synthesis for example of the fatty acid biosynthesis, the polypeptide of the invention or used in the method of the invention, the nucleic acid construct of the invention, the expression cassette of the invention, the vector of the invention, the plant or plant tissue or the host cell of the invention, for the production of plant resistant to a herbicide inhibiting eventually the production of the fine chemical.

Furthermore preferred is a method for the recombinant production of fine chemicals in non-human host organisms, wherein a non-human host organism is transformed with one of the above-described nucleic acid molecules or nucleic acid constructs, expression cassettes or vectors comprising one or more nucleic acid molecules, wherein said nucleic acid molecule participates in the biosynthesis of the desired fine chemical or encodes a polypeptide that catalyze the biosynthesis of the desired fine chemical, the transformed non-human host organism is cultured, and the desired fine chemical is isolated from the non-human organism and/or culture medium. In another embodiment the additional production of further fatty acids or compositions comprising said compounds and/or of pharmaceuticals is preferred. The transformed non-human host organisms are cultured and the products are recovered from the non-human host organisms and/or the culture medium by methods known to the skilled worker or the non-human organism itself serves as food or feed or food or feed supplement. The production of pharmaceuticals such as, for example, antibodies or vaccines, is described by Hood E. E. et al., J, Curr. Opin. Biotechnol. 10 (4), 382 (1999), Ma J. K., Vine N. D., Curr. Top. Microbiol. Immunol. 236, 275 (1999).

In one embodiment, the present invention relates to a method for the identification of a gene product conferring the production of or an increase in the fine chemical production in a non-human cell, comprising the following steps:
(a) contacting, e.g. hybridizing, a, some or all nucleic acid molecules of a sample, e.g. cells, tissues, plants or microorganisms or a nucleic acid library, which can contain a candidate gene encoding a gene product conferring the production of or an increase in the fine chemical after expression, with the nucleic acid molecule of the present invention as shown in the respective line in column 5 or 8 of Table I A or B of application no. 8, preferably the coding region thereof, or a fragment or homolog thereof;
(b) identifying the nucleic acid molecules, which hybridize under relaxed stringent conditions with the nucleic acid molecule of the present invention in particular to the nucleic acid molecule sequence shown in the respective line in Table I, application no. 8, columns 5 or 8, preferably the coding region thereof, of a fragment or a homolog thereof, preferably in Table I B, application no. 8, columns 5 or 8, preferably the coding region thereof, of a fragment or a homolog thereof, and, optionally, isolating the full length cDNA clone or complete genomic clone;
(c) introducing the candidate nucleic acid molecules or a fragment thereof in host cells, preferably in a plant cell or a microorganism, appropriate for producing the fine chemical;
(d) expressing the identified nucleic acid molecules in the host cells for which the production of or the increased production of the fine chemical is desired;
(e) assaying the fine chemical level in the non-human host cells; and
(f) identifying the nucleic acid molecule and/or its gene product which expression confers the production of or an increase in the fine chemical level in the non-human host cell after expression compared to the wild type.

Relaxed hybridization conditions are: After standard hybridization procedures washing steps can be performed at low to medium stringency conditions usually with washing conditions of 40° -55° C. and salt conditions between 2×SSC and 0.2×SSC with 0.1% SDS in comparison to stringent washing conditions as e.g. 60° -68° C. with 0.1% SDS. Further examples can be found in the references listed above for the stringent hybridization conditions. Usually washing steps are repeated with increasing stringency and length until a useful signal to noise ratio is detected and depend on many factors as the target, e.g. its purity, GC-content, size etc, the probe, e.g. its length, is it a RNA or a DNA probe, salt conditions, washing or hybridization temperature, washing or hybridization time etc.

In another embodiment, the present invention relates to a method for the identification of a gene product conferring the production of or an increase in the fine chemical production in a cell, comprising the following steps:
(a) identifying a nucleic acid molecule of a non-human organism, which is at least 20%, 25%, 30%, 35%, 40%, 50%, 60%, 70%, 80%, 90%, 95%, 96%, 97%, 98%, 99%, 99.5% or more homolog to the nucleic acid molecule encoding a polypeptide comprising the polypeptide molecule as shown in the respective line in column 5 or 8 of Table II, application no. 8, or comprising a consensus sequence or a polypeptide motif as shown in the respective line in column 8 of Table IV, application no. 8, or being encoded by a nucleic acid molecule comprising a polynucleotide as shown in the respective line in column 5 or 8 of Table I, application no. 8, preferably the coding region thereof, or a homologue thereof as described herein, for example via homology search in a data bank;
(b) introducing the candidate nucleic acid molecule in non-human host cells, preferably in a plant cell or a microorganism, appropriate for producing the fine chemical;
(c) expressing the identified nucleic acid molecule in the non-human host cells;
(d) assaying the fine chemical level in the non-human host cells; and
(e) identifying the nucleic acid molecule and its gene product which expression confers the production of or an increase in the fine chemical level in the non-human host cell after expression compared to the wild type.

The nucleic acid molecules identified can then be used for the production of the fine chemical in the same way as the nucleic acid molecule of the present invention. Accordingly, in one embodiment, the present invention relates to a process for the production of the fine chemical, comprising (i) identifying a nucleic acid molecule according to aforementioned steps (a) to (f) or (a) to (e) and (ii) recovering the free or bound fine chemical from a non-human organism having an increased cellular activity of a polypeptide encoded by the nucleic acid molecule compared to a wild type and/or the respective culture medium.

Further, the nucleic acid molecules disclosed herein, in particular the nucleic acid molecules shown in the respective line in column 5 or 8 of Table I A or B of application no. 8, may be sufficiently homologous to the sequences of related species such that these nucleic acid molecules may serve as markers for the construction of a genomic map in related organism or for association mapping. Furthermore natural variation in the genomic regions corresponding to nucleic acids disclosed herein, in particular the nucleic acid molecule shown in the respective line in column 5 or 8 of Table I A or B, of application no. 8 or homologous thereof, may lead to variation in the activity of the proteins disclosed herein, in particular the proteins comprising polypeptides as shown in the respective lines in column 5 or 8 of Table II A or B, of application no. 8 or their homologous, or comprising the consensus sequence or the polypeptide motif as shown in the respective line in column 8 of Table IV, and in consequence in a natural variation of an increased fine chemical production.

In consequence natural variation eventually also exists in form of more active allelic variants leading already to a relative increase in the fine chemical. Different variants of the nucleic acid molecules disclosed herein, in particular the nucleic acid molecules comprising the nucleic acid molecules as shown in the respective line in column 5 or 8 of Table I A or B of application no. 8, preferably the coding region thereof, which corresponds to different levels of increase in fine chemical, e.g. different levels of increase in fine chemical, can be indentified and used for marker assisted breeding for an enhanced production of the fine chemical.

Natural variation may also exist in the regulatory regions, e.g. the promoters of the natural genes comprising the nucleic acid molecules as shown in the respective line column 5 or 8 of Table I A or B of application no. 8, preferably the coding region thereof, which can similarly correspond to different levels of increase in the fine chemical, e.g. different levels of increase in the fine chemical can be identified and used for marker assisted breeding for an enhanced production of the fine chemical.

Accordingly, the present invention relates to a method for breeding plants for the production of the fine chemical, comprising
(a) selecting a first plant variety with an increased production of the fine chemical, e.g. based on an increased expression of a nucleic acid molecule of the invention as disclosed herein, in particular of a nucleic acid molecule comprising a nucleic acid molecule as shown in the respective line in column 5 or 8 of Table I A or B of application no. 8, preferably the coding region thereof, or fragments or homologs thereof, or a polypeptide comprising a polypeptide as shown in the respective line in column 5 or 8 of Table II A or B of application no. 8, or fragments or homologs thereof, or comprising a consensus sequence or a polypeptide motif as shown in the respective line in column 8 of Table IV, or a homolog thereof, as described herein;
(b) associating the level of increased production of the fine chemical, with the expression level or the genomic structure of a gene encoding said polypeptide or said nucleic acid molecule;
(c) crossing the first plant variety with a second plant variety, which significantly differs in its level of fine chemical; and
(d) identifying, which of the offspring varieties has got increased production of the fine chemical by the expression level of said polypeptide or nucleic acid molecule or the genomic structure of the gene(s) encoding said polypeptide or nucleic acid molecule of the invention.

In one embodiment, the expression level of the gene according to step (b) is increased.

In another embodiment, the present invention relates to a method for the identification of a compound stimulating production of the fine chemical in a non-human organism, especially a microorganism or a plant, comprising:
(a) contacting the microorganism or plant cells which express the polypeptide of the present invention or its mRNA with a candidate compound under appropriate conditions or in case of plant cells under cell cultivation conditions;
(b) assaying the production of or an increase in expression of said polypeptide or said mRNA;
(c) comparing the expression level to a standard response made in the absence of said candidate compound; whereby, the production of or an increased expression over the standard indicates that the compound is stimulating production of the fine chemical.

Furthermore, in one embodiment, the present invention relates to a process for the identification of a compound conferring the production of or an increase in the fine chemical production in a non-human organism, especially a plant or microorganism, comprising the steps:
(a) culturing a cell or a part of a plant or a microorganism or maintaining a plant expressing the polypeptide according to the invention or a nucleic acid molecule encoding said polypeptide and a readout system capable of interacting with the polypeptide under suitable conditions which permit the interaction of the polypeptide with said readout system in the presence of a compound or a sample comprising a plurality of compounds and capable of providing a detectable signal in response to the binding of a compound to said polypeptide under conditions which permit the expression of said readout system and the polypeptide of the present invention or used in the process of the invention; and
(b) identifying if the compound is an effective agonist by detecting the presence or absence or increase of a signal produced by said readout system.

The screen for a gene product or an agonist conferring the production of or an increase in the fine chemical can be performed by growth of a non-human organism for example a microorganism in the presence of growth reducing amounts of an inhibitor of the synthesis of the fine chemical. Better growth, e.g. higher dividing rate or high dry mass in comparison to the control, e.g. with a gene product or an agonist conferring the production of or an increase in the fine chemical, under such conditions would identify a gene or gene product or an agonist conferring the production of or an increase in fine chemical.

Said compound may be chemically synthesized or microbiologically produced and/or comprised in, for example, samples, e.g., cell extracts from, e.g., plants, non-human animals or microorganisms, e.g. pathogens. Furthermore, said compound(s) may be known in the art but hitherto not known to be capable of suppressing or activating the polypeptide of the present invention. The reaction mixture may be a cell free extract or may comprise a cell or tissue culture. Suitable set ups for the method of the invention are known to the person skilled in the art and are, for example, generally described in Alberts et al., “Molecular Biology of the Cell”, third edition (1994), in particular Chapter 17. The compounds may be, e.g., added to the reaction mixture, culture medium, injected into the cell or sprayed onto the plant.

If a sample containing a compound is identified in the method of the invention, then it is either possible to isolate the compound from the original sample identified as containing the compound capable of activating or conferring the production of or an increase in the fine chemical in a non-human organism or part thereof, or one can further subdivide the original sample, for example, if it consists of a plurality of different compounds, so as to reduce the number of different compounds per sample and repeat the method with the subdivisions of the original sample. Depending on the complexity of the samples, the steps described above can be performed several times, preferably until the sample identified according to the method of the invention only comprises a limited number of or only one compound(s). Preferably said sample comprises compounds of similar chemical and/or physical properties. Preferably, the compound identified according to the above-described method or a respective “active” derivative thereof is further formulated in a form suitable for the application in plant breeding or plant cell and tissue culture.

The compounds which can be tested and identified according to a method of the invention may be expression libraries, e.g., cDNA expression libraries, peptides, proteins, nucleic acids, antibodies, small organic compounds, hormones, peptidomimetics, PNAs or the like (Milner, Nature Medicine 1, 879 (1995); Hupp, Cell 83, 237 (1995); Gibbs, Cell 79, 193 (1994) and references cited supra). Said compounds can also be functional derivatives or analogues of known inhibitors or activators. Methods for the preparation of chemical derivatives and analogues are well known to those skilled in the art and are described in, for example, Beilstein,

Handbook of Organic Chemistry, Springer edition New York Inc., 175 Fifth Avenue, New York, N.Y. 10010 U.S.A. and Organic Synthesis, Wiley, New York, USA. Furthermore, said derivatives and analogues can be tested for their effects according to methods known in the art. Furthermore, peptidomimetics and/or computer aided design of appropriate derivatives and analogues can be used, for example, according to the methods described above. The non-human cell or tissue that may be employed in the method of the invention preferably is a non-human host cell, plant cell or plant tissue of the invention described in the embodiments hereinbefore.

Thus, in a further embodiment the invention relates to a compound obtained or identified according to the method for identifying an agonist of the invention said compound being an agonist of the polypeptide of the present invention or used in the process of the present invention.

Accordingly, in one embodiment, the present invention further relates to a compound identified by the method for identifying a compound of the present invention.

Said compound is, for example, a homolog of the polypeptide of the present invention. Homologs of the polypeptide of the present invention can be generated by mutagenesis, e.g., discrete point mutation or truncation of the polypeptide of the present invention. As used herein, the term “homolog” refers to a variant form of the protein, which acts as an agonist of the activity of the polypeptide of the present invention. An agonist of said protein can retain substantially the same, or a subset, of the biological activities of the polypeptide of the present invention. In particular, said agonist confers the increase of the expression level of the polypeptide of the present invention and/or the expression of said agonist in a non-human organisms or part thereof confers the increase of free and/or bound fine chemical in the non-human organism or part thereof.

In another embodiment, the invention relates to an antibody specifically recognizing the compound or agonist of the present invention.

The invention also relates to a diagnostic composition comprising at least one of the aforementioned nucleic acid molecules, expression cassettes, vectors, proteins, antibodies or compounds of the invention and optionally suitable means for detection.

The diagnostic composition of the present invention is suitable for the isolation of mRNA from a non-human cell and contacting the mRNA so obtained with a probe comprising a nucleic acid probe as described above under hybridizing conditions, detecting the presence of mRNA hybridized to the probe, and thereby detecting the expression of the protein in the non-human cell. Further methods of detecting the presence of a protein according to the present invention comprise immunotechniques well known in the art, for example enzyme linked immuno-adsorbent assay. Furthermore, it is possible to use the nucleic acid molecules according to the invention as molecular markers or primers in plant breeding. Suitable means for detection are well known to a person skilled in the art, e.g. buffers and solutions for hybridization assays, e.g. the afore-mentioned solutions and buffers, and further means for Southern-, Western-, Northern- etc. -blots, as e.g. described in Sambrook et al. are known. In one embodiment diagnostic compositions contain PCR primers designed to specifically detect the presence or the expression level of the nucleic acid molecule to be expressed or to be expressed on an enhanced level in the process of the invention, e.g. of the nucleic acid molecule of the invention, or to discriminate between different variants or alleles of the nucleic acid molecule of the invention or nucleic acid molecules the activity of which is to be increased in the process of the invention.

In another embodiment, the present invention relates to a kit comprising the nucleic acid molecule, the vector, the host cell, the polypeptide, or the, viral nucleic acid molecule, antibody, plant cell, plant or part thereof, the harvestable part, the propagation material and/or the compound and/or agonist identified according to the method of the invention.

The compounds of the kit of the present invention may be packaged in containers such as vials, optionally with/in buffers and/or solvents. If appropriate, one or more of the components of said kits might be packaged in one and the same container or in different ones. Additionally or alternatively, one or more of said components might be adsorbed to a solid support, like a nitrocellulose filter, a glass plate, a chip, a nylon membrane or to the well of a micro titer plate. The kit can be used for any of the herein described methods and embodiments, e.g.

for the production of the host cells, transgenic plants, pharmaceutical compositions, detection of homologous sequences, identification of agonists, as food or feed or as a supplement thereof or as supplement for the treating of plants, etc. Further, the kit can comprise instructions for the use of the kit for any of said embodiments. In one embodiment said kit comprises further a nuoleic acid molecule encoding one or more of the aforementioned protein, and/or an antibody, a vector, a host cell, a plant cell, a plant or a part thereof. In another embodiment said kit comprises PCR primers to detect and increase the nucleic acid molecule to be increased in the process of the invention, e.g. of the nucleic acid molecule of the invention.

In a further embodiment, the present invention relates to a method for the production of a agricultural composition providing the nucleic acid molecule, the vector, or antibody of the present invention, the viral nucleic acid of the invention, or the polypeptide of the invention, the compound or agonist or comprising the steps of the method according to the invention for the identification of said compound or agonist; and formulating the nucleic acid molecule, the vector, or antibody of the present invention, the viral nucleic acid of the invention, the polypeptide of the invention or the agonist, or compound identified according to the methods or processes of the present invention or with use of the subject matters of the present invention in a form applicable as plant agricultural composition.

In another embodiment, the present invention relates to a method for the production of “the fine chemical”-production supporting plant culture composition comprising the steps of the method of the present invention; and formulating the compound identified in a form acceptable as agricultural composition.

Under “acceptable as agricultural composition” is understood, that such a composition optionally comprises auxiliaries which are customarily used for formulating crop protection agents.

In a further embodiment the nucleic acid molecule of the invention, the polypeptide of the invention, the nucleic acid construct of the invention, the expression cassette of the invention, the non-human organisms, the non-human host cell, the microorganisms, the plant, plant cell, or the part thereof, of the invention, the vector of the invention, the agonist identified with the method of the invention, the nucleic acid molecule identified with the method of the present invention, can be used for the production of the fine chemical or of the fine chemical and one or more other fatty acids, e.g. polyunsaturated long-chain w-3-fatty acids such as eicosapentaenoic acid (=EPA) or docosahexaenoic acid (=DHA)

Accordingly, the nucleic acid molecule of the invention, or the nucleic acid molecule identified with the method of the present invention or the complement sequences thereof, the polypeptide of the invention, the nucleic acid construct of the invention, the expression cassette of the invention, the non-human organism, the non-human host cell, the microorganism, the plant, plant cell, or the part thereof of the invention, the vector of the invention, the agonist identified with the method of the invention, the antibody of the present invention, can be used for the production of or the increase in the fine chemical in a non-human organism or part thereof, e.g. in a cell.

These and other embodiments are disclosed and encompassed by the description and examples of the present invention. Further literature concerning any one of the methods, uses and compounds to be employed in accordance with the present invention may be retrieved from public libraries, using for example electronic devices. For example the public database “Medline” may be utilized which is available on the Internet, for example under http://www.ncbi.nlm.nih.gov/PubMed/medline.html. Further databases and addresses, such as http://www.ncbi.nlm.nih.gov/, http://www.infobiogen.fr/, http://www.fmi.ch/biology/researchtools.html, hftp://www.tigr.org/, are known to the person skilled in the art and can also be obtained using, e.g., http://www.lycos.com.

The present invention is illustrated by the examples, which follow. The present examples illustrate the basic invention without being intended as limiting the subject of the invention. The content of all of the references, patent applications, patents and published patent applications cited in the present patent application is herewith incorporated by reference.

A further embodiment of this invention is related to genes which increase or generate the production of the fine chemical linoleic acid in plant cells, plants or part thereof. Phenotypes thereto are associated with yield of plants (=yield related phenotypes). In accordance with the invention, therefore, the respective genes identified in Table I, wherein in column 7 linoleic acid is mentioned, especially the coding region thereof, or homologs or fragments thereof, may be employed to enhance any yield-related phenotype. Increased yield may be determined in field trials of transgenic plants and suitable control plants. Alternatively, a transgene's ability to increase yield may be determined in a model plant. An increased yield phenotype may be determined in the field test or in a model plant by measuring any one or any combination of the following phenotypes, in comparison to a control plant: yield of dry harvestable parts of the plant, yield of dry aerial harvestable parts of the plant, yield of underground dry harvestable parts of the plant, yield of fresh weight harvestable parts of the plant, yield of aerial fresh weight harvestable parts of the plant yield of underground fresh weight harvestable parts of the plant, yield of the plant's fruit (both fresh and dried), grain dry weight, yield of seeds (both fresh and dry), and the like.

The most basic yield-related phenotype is increased yield associated with the presence of the gene or a homolog or a fragment thereof as a transgene in the plant, i.e., the intrinsic yield of the plant. Intrinsic yield capacity of a plant can be, for example, manifested in a field test or in a model system by demonstrating an improvement of seed yield (e.g. in terms of increased seed/grain size, increased ear number, increased seed number per ear, improvement of seed filling, improvement of seed composition, embryo and/or endosperm improvements, and the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under non-stressed conditions), improvement in plant architecture. In accordance with the invention, the respective genes identified in Table I, application 8, especially the coding region thereof, or homologs or fragments thereof, wherein in the respective line linoleic acid is mentioned, may be employed to enhance intrinsic yield capacity.

Increased yield-related phenotypes may also be measured to determine tolerance to abiotic environmental stress. Abiotic stresses include drought, low temperature, nutrient deficiency, salinity, osmotic stress, shade, high plant density, mechanical stresses, and oxidative stress, and yield-related phenotypes are encompassed by tolerance to such abiotic stresses. Additional phenotypes that can be monitored to determine enhanced tolerance to abiotic environmental stress include, without limitation, wilting; leaf browning; loss of turgor, which results in drooping of leaves or needles stems, and flowers; drooping and/or shedding of leaves or needles; the leaves are green but leaf angled slightly toward the ground compared with controls; leaf blades begun to fold (curl) inward; premature senescence of leaves or needles; loss of chlorophyll in leaves or needles and/or yellowing. Any of the yield-related phenotypes described above may be monitored in field tests or in model plants to demonstrate that a transgenic plant has increased tolerance to abiotic environmental stress.

A polypeptide conferring an yield-increasing activity can be encoded by a respective nucleic acid sequence as shown in Table I, column 5 or 8, and/or comprises or consists of a respective polypeptide as depicted in Table II, column 5 and 8, and/or can be amplified with the respective primer set shown in Table III, column 8, in case in column 8 linoleic acid is indicated.

“Improved adaptation” to environmental stress like e.g. freezing and/or chilling temperatures refers to an improved plant performance under environmental stress conditions.

A modification, i.e. an increase, can be caused by endogenous or exogenous factors. For example, an increase in activity in an organism or a part thereof can be caused by adding a gene product or a precursor or an activator or an agonist to the media or nutrition or can be caused by introducing said subjects into an organism, transient or stable. Furthermore such an increase can be reached by the introduction of the respective inventive nucleic acid sequence or the encoded protein in the correct cell compartment for example into the nucleus or cytoplasmic respectively or into plastids either by transformation and/or targeting.

The term “yield” as used herein generally refers to a measurable produce from a plant, particularly a crop. Yield and yield increase (in comparison to a non-transformed starting or wild-type plant) can be measured in a number of ways, and it is understood that a skilled person will be able to apply the correct meaning in view of the particular embodiments, the particular crop concerned and the specific purpose or application concerned. The terms “improved yield” or “increased yield” can be used interchangeable.

As used herein, the term “improved yield” or the term “increased yield” means any improvement in the yield of any measured plant product, such as grain, fruit or fiber. In accordance with the invention, changes in different phenotypic traits may improve yield. For example, and without limitation, parameters such as floral organ development, root initiation, root biomass, seed number, seed weight, harvest index, tolerance to abiotic environmental stress, leaf formation, phototropism, apical dominance, and fruit development, are suitable measurements of improved yield. Any increase in yield is an improved yield in accordance with the invention. For example, the improvement in yield can comprise a 0.1%, 0.5%, 1%, 3%, 5%, 10%, 15%, 20%, 30%, 40%, 50%, 60%, 70%, 80%, 90% or greater increase in any measured parameter. For example, an increase in the bushel/acre yield of soybeans or corn derived from a crop comprising plants which are transgenic for the nucleotides and polypeptides of Table I, in case in column 7 linoleic acid is mentioned, as compared with the bushel/acre yield from untreated soybeans or corn cultivated under the same conditions, is an improved yield in accordance with the invention. The increased or improved yield can be achieved in the absence or presence of stress conditions.

For example, enhanced or increased “yield” refers to one or more yield parameters selected from the group consisting of biomass yield, dry biomass yield, aerial dry biomass yield, underground dry biomass yield, fresh-weight biomass yield, aerial fresh-weight biomass yield, underground fresh-weight biomass yield; enhanced yield of harvestable parts, either dry or fresh-weight or both, either aerial or underground or both; enhanced yield of crop fruit, either dry or fresh-weight or both, either aerial or underground or both; and preferably enhanced yield of seeds, either dry or fresh-weight or both, either aerial or underground or both.

Crop yield is defined herein as the number of bushels of relevant agricultural product (such as grain, forage, or seed) harvested per acre. Crop yield is impacted by abiotic stresses, such as drought, heat, salinity, and cold stress, and by the size (biomass) of the plant. Traditional plant breeding strategies are relatively slow and have in general not been successful in conferring increased tolerance to abiotic stresses. Grain yield improvements by conventional breeding have nearly reached a plateau in maize.

Accordingly, the yield of a plant can depend on the specific plant/crop of interest as well as its intended application (such as food production, feed production, processed food production, biofuel, biogas or alcohol production, or the like) of interest in each particular case. Thus, in one embodiment, yield is calculated as harvest index (expressed as a ratio of the weight of the respective harvestable parts divided by the total biomass), harvestable parts weight per area (acre, square meter, or the like); and the like. The harvest index, i.e., the ratio of yield biomass to the total cumulative biomass at harvest, in maize has remained essentially unchanged during selective breeding for grain yield over the last hundred years. Accordingly, recent yield improvements that have occurred in maize are the result of the increased total biomass production per unit land area. This increased total biomass has been achieved by increasing planting density, which has led to adaptive phenotypic alterations, such as a reduction in leaf angle, which may reduce shading of lower leaves, and tassel size, which may increase harvest index. Harvest index is relatively stable under many environmental conditions, and so a robust correlation between plant size and grain yield is possible. Plant size and grain yield are intrinsically linked, because the majority of grain biomass is dependent on current or stored photosynthetic productivity by the leaves and stem of the plant. As with abiotic stress tolerance, measurements of plant size in early development, under standardized conditions in a growth chamber or greenhouse, are standard practices to measure potential yield advantages conferred by the presence of a transgene.

Acccordingly, the yield of a plant can be increased by improving one or more of the yield-related phenotypes.

Such yield-related phenotypes or traits of a plant the improvement of which results in increased yield comprise, without limitation, the increase of the intrinsic yield capacity of a plant, and/or increased stress tolerance, e.g improved drought tolerance or improved nutrient use efficiency.

For example, yield refers to biomass yield, e.g. to dry weight biomass yield and/or fresh-weight biomass yield. Biomass yield refers to the aerial or underground parts of a plant, depending on the specific circumstances (test conditions, specific crop of interest, application of interest, and the like). In one embodiment, biomass yield refers to the aerial and underground parts. Biomass yield may be calculated as fresh-weight, dry weight or a moisture adjusted basis. Biomass yield may be calculated on a per plant basis or in relation to a specific area (e.g. biomass yield per acre/ square meter/ or the like).

In other embodiment, “yield” refers to seed yield which can be measured by one or more of the following parameters: number of seeds or number of filled seeds (per plant or per area (acre/square meter/ or the like)); seed filling rate (ratio between number of filled seeds and total number of seeds); number of flowers per plant; seed biomass or total seeds weight (per plant or per area (acre/square meter/ or the like); thousand kernel weight (TKW; extrapolated from the number of filled seeds counted and their total weight; an increase in TKW may be caused by an increased seed size, an increased seed weight, an increased embryo size, and/or an increased endosperm). Other parameters allowing to measure seed yield are also known in the art. Seed yield may be determined on a dry weight or on a fresh weight basis, or typically on a moisture adjusted basis, e.g. at 15.5 percent moisture.

For example, the term “increased yield” means that the a plant, exhibits an increased growth rate, under conditions of abiotic environmental stress, compared to the corresponding wild-type plant.

An increased growth rate may be reflected inter alia by or confers an increased biomass production of the whole plant, or an increased biomass production of the aerial parts of a plant, or by an increased biomass production of the underground parts of a plant, or by an increased biomass production of parts of a plant, like stems, leaves, blossoms, fruits, and/or seeds.

Increased yield includes higher fruit yields, higher seed yields, higher fresh matter production, and/or higher dry matter production.

The term “increased yield” means that the plant, exhibits a prolonged growth under conditions of abiotic environmental stress, as compared to the corresponding, e.g. non-transformed, wild type organism. A prolonged growth comprises survival and/or continued growth of the plant, at the moment when the non-transformed wild type organism shows visual symptoms of deficiency and/or death.

When the plant of the invention is a corn plant increased yield for corn plants means, for example, increased seed yield, in particular for corn varieties used for feed or food. Increased seed yield of corn refers to an increased kernel size or weight, an increased kernel per ear, or increased ears per plant. Alternatively or in addition the cob yield may be increased, or the length or size of the cob is increased, or the kernel per cob ratio is improved.

When the plant of the invention is a soy plant, increased yield for soy plants means increased seed yield, in particular for soy varieties used for feed or food. Increased seed yield of soy refers for example to an increased kernel size or weight, an increased kernel per pod, or increased pods per plant.

When the plant of the invention is an oil seed rape (OSR) plant, increased yield for OSR plants means increased seed yield, in particular for OSR varieties used for feed or food. Increased seed yield of OSR refers to an increased seed size or weight, an increased seed number per silique, or increased siliques per plant.

When the plant of the invention is a cotton plant, increased yield for cotton plants means increased lint yield. Increased lint yield of cotton refers in one embodiment to an increased length of lint.

Said increased yield can typically be achieved by enhancing or improving, one or more yield-related traits of the plant. Such yield-related traits of a plant comprise, without limitation, the increase of the intrinsic yield capacity of a plant, and/or increased stress tolerance, in particular increased abiotic stress tolerance, like for example improved nutrient use efficiency, e.g. nitrogen use efficiency, water use efficiency.

Intrinsic yield capacity of a plant can be, for example, manifested by improving the specific (intrinsic) seed yield (e.g. in terms of increased seed/ grain size, increased ear number, increased seed number per ear, improvement of seed filling, improvement of seed composition, embryo and/or endosperm improvements, or the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under stressed or non-stressed conditions), improvement in plant architecture, cell cycle modifications, photosynthesis modifications, various signaling pathway modifications, modification of transcriptional regulation, modification of translational regulation, modification of enzyme activities, and the like); and/or the like.

The improvement or increase of stress tolerance of a plant can for example be manifested by improving or increasing a plant's tolerance against stress, particularly abiotic stress. In the present application, abiotic stress refers generally to abiotic environmental conditions a plant is typically confronted with, including, but not limited to, drought (tolerance to drought may be achieved as a result of improved water use efficiency), heat, low temperatures and cold conditions (such as freezing and chilling conditions), nutrient depletion, salinity, osmotic stress, shade, high plant density, mechanical stress, oxidative stress, and the like.

The increased plant yield can for example be mediated by increasing the “nutrient use efficiency of a plant”, e.g. by improving the use efficiency of nutrients including, but not limited to, phosphorus, potassium, and nitrogen. Further, higher yields may be obtained with current or standard levels of nitrogen use.

Generally, the term “increased tolerance to stress” can be defined as survival of plants, and/or higher yield production, under stress conditions as compared to a non-transformed wild type or starting plant: For example, the plant of the invention or produced according to the method of the invention is better adapted to the stress conditions. “Improved adaptation” to environmental stress like e.g. drought, heat, nutrient depletion, freezing and/or chilling temperatures refers herein to an improved plant performance resulting in an increased yield, particularly with regard to one or more of the yield related traits as defined in more detail above.

During its life-cycle, a plant is generally confronted with a diversity of environmental conditions. Any such conditions, which may, under certain circumstances, have an impact on plant yield, are herein referred to as “stress” condition. Environmental stresses may generally be divided into biotic and abiotic (environmental) stresses. Unfavorable nutrient conditions are also referred to as “environmental stress”. The present invention does also contemplate solutions for this kind of environmental stress, e.g. referring to increased nutrient use efficiency.

For the purposes of the description of the present invention, the terms “enhanced tolerance to abiotic stress”, “enhanced resistance to abiotic environmental stress”, “enhanced tolerance to environmental stress”, “improved adaptation to environmental stress” and other variations and expressions similar in its meaning are used interchangeably and refer, without limitation, to an improvement in tolerance to one or more abiotic environmental stress(es) as described herein and as compared to a corresponding origin or wild type plant or a part thereof.

The term abiotic stress tolerance(s) refers for example low temperature tolerance, drought tolerance or improved water use efficiency (WUE), heat tolerance, salt stress tolerance, improved nutrient efficiency, especially nitrogen use efficiency (NUE) and others. Studies of a plant's response to desiccation, osmotic shock, and temperature extremes are also employed to determine the plant's tolerance or resistance to abiotic stresses. Water use efficiency (WUE) is a parameter often correlated with drought tolerance. In selecting traits for improving crops, a decrease in water use, without a change in growth would have particular merit in an irrigated agricultural system where the water input costs were high. An increase in growth without a corresponding jump in water use would have applicability to all agricultural systems. In many agricultural systems where water supply is not limiting, an increase in growth, even if it came at the expense of an increase in water use also increases yield.

Drought stress means any environmental stress which leads to a lack of water in plants or reduction of water supply to plants, including a secondary stress by low temperature and/or salt, and/or a primary stress during drought or heat, e.g. desiccation etc.

### Specific Embodiments

Accordingly, this invention provides respective measures and methods to produce plants with increased yield, e.g. genes conferring an increased yield-related trait, for example enhanced tolerance to abiotic environmental stress, for example an increased drought tolerance and/or low temperature tolerance and/or an increased nutrient use efficiency, intrinsic yield and/or another increased yield-related trait, upon expression or over-expression, especially increased intrinsic yield. Accordingly, the present invention provides such genes in case in column 7 of the respective Table “linoleic acid” is indicated. In particular, such genes are described in column 5 as well as in column 8 of Tables I, especially the coding region thereof, or homologs or fragments thereof, in case linoleic acid is indicated in column 7 or the respective polypeptides are described in column 5 as well as in column 8 of Table II, or homologs or fragments thereof, in case linoleic acid is indicated in column 7.

Accordingly, the present invention provides respective transgenic plants showing one or more improved yield-related traits as compared to the corresponding origin or the wild type plant and methods for producing such transgenic plants with increased yield in case in column 7 of the respective Table “linoleic acid” is indicated.

In one embodiment, one or more of said yield-increasing activities are increased by increasing the amount and/or the specific activity of one or more proteins listed in Table I, column 5 or 8 in a compartment of a cell indicated in Table I, column 6, in case in column 7 of the respective Table “linoleic acid” is indicated.

Accordingly to present invention, the yield of the plant of the invention is increased by improving one or more of the yield-related traits as defined herein. Said increased yield in accordance with the present invention can typically be achieved by enhancing or improving, in comparison to an origin or wild-type plant, one or more yield-related traits of said plant. Such yield-related traits of a plant the improvement of which results in increased yield comprise, without limitation, the increase of the intrinsic yield capacity of a plant, and/or increased stress tolerance, e.g. improved nutrient use efficiency, like nitrogen use efficiency; especially enhanced intrinsic yield capacity.

In another embodiment the present invention relates to plants with enhanced intrinsic yield capacity in case a plant is transformed with a gene being depicted in Table I, and wherein in the same line of said gene in column 7 linoleic acid is depicted.

In a further embodiment the present invention is characterized by the features as disclosed in the following items:

Item 1. A process for the production of the fine chemical linoleic acid
(a) increasing or generating one or more activities selected from the group consisting of 3-ketoacyl-CoA thiolase, 3-oxoacyl-[acyl-carrier-protein] synthase, AAS bifunctional protein, ABC transporter permease protein, acid shock protein, aconitate hydratase, arginine decarboxylase, aromatic acid decarboxylase, aspartate-semialdehyde dehydrogenase, At1g19800-protein, At2g45420-protein, ATPase epsilon subunit, b0801-protein, b1330-protein, b1470-protein, b1670-protein, b1837-protein, b2032-protein, b2099-protein, b2107-protein, b2399-protein, b2474-protein, b2613-protein, b2682-protein, b2849-protein, b2909-protein, beta-hydroxylase, carbamoylphosphate synthase subunit, cation-transporting ATPase, CCAAT-binding transcription factor, chlorophyllase, colanic acid biosynthesis protein, coproporphyrinogen III oxidase, CTP synthetase, DnaJ-like chaperone, electron transport complex protein, ethanolamine utilization protein, fatty acid desaturase, Fe—S subunit of oxidoreductase, flavodoxin, fumarate hydratase, gamma-glutamyltranspeptidase, glucose-6-phosphate 1-dehydrogenase, glutaredoxin, glycin cleavage system protein, GMO2LC38418-protein, heat shock transcription factor, hydrolase, lipoprotein precursor, membrane transport protein, metal-dependent hydrolase, molecular chaperone, monothiol glutaredoxin, multiple antibiotic resistance protein, outer membrane receptor protein precursor, oxidoreductase, phosphate import ATP-binding protein, phosphoribosylformyl glycinamidine synthase subunit, phosphotriesterase, protein kinase, protein phosphatase, pyruvate kinase, recombinase A, sll1032-protein, sn-glycerol-3-phosphate transport system permease protein, ThiF family protein, threonine dehydratase, threonine efflux protein, transcription factor, transcriptional regulator protein, tryptophan synthase alpha chain, TTC0490-protein, xanthine dehydrogenase, ydr507c-protein, and YGR104C-protein, in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linoleic acid or a composition comprising linoleic acid in said non-human organism or in the culture medium surrounding said non-human organism.

Item 2. A process for the production of a respective fine chemical linoleic acid, which comprises
(A)
   (i) increasing or generating of the expression of; and/or
   (ii) increasing or generating the expression of an expression product of; and/or
   (iii) increasing or generating one or more activities of an expression product encoded by;
   at least one respective nucleic acid molecule comprising a nucleic acid molecule selected from the group consisting of:
   (a) a nucleic acid molecule encoding the polypeptide shown in column 5 or 8 of Table II, preferably Table II B, application no. 8, or a homolog or a fragment thereof;
   (b) a nucleic acid molecule shown in column 5 or 8 of Table I, preferably Table I B, application no.8, or a homolog or a fragment thereof (preferably the coding region thereof);
   (c) a nucleic acid molecule, which, as a result of the degeneracy of the genetic code, can be derived from a polypeptide sequence depicted in column 5 or 8 of Table II, preferably Table II B, application no. 8;
   (d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 8, or the coding region thereof;
   (e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 8;
   (i) a nucleic acid molecule encoding a polypeptide having the activity represented by a respective protein as depicted in column 5 of Table II, application no. 8;
   (j) a nucleic acid molecule which comprises a polynucleotide, which is obtained by amplifying a cDNA library or a genomic library using the primers as depicted in column 8 of Table III, application no. 8; and
   (k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
   or a nucleic acid molecule comprising a sequence which is complementary thereto; in a non-human organism or a part thereof, preferably a microorganism, a plant cell, a plant or a part thereof, as compared to a corresponding non-transformed wild type non-human organism or a part thereof; and
(B) growing the non-human organism or a part thereof under conditions which permit the production of linoleic acid or a composition comprising linoleic acid in said non-human organism or in the culture medium surrounding said non-human organism.

Item 3. A process of items 1 or 2, comprising of recovering linoleic acid in its free or bound form.

Item 4. The process of any one of items 1 to 3, comprising the following steps:
(a) selecting a non-human organism or a part thereof expressing a polypeptide encoded by the nucleic acid molecule characterized in item 2;
(b) mutagenizing the selected non-human organism or the part thereof;
(c) comparing the activity or the expression level of said polypeptide in the mutagenized non-human organism or the part thereof with the activity or the expression of said polypeptide of the selected non-human organisms or the part thereof;
(d) selecting the mutated non-human organisms or parts thereof, which comprise an increased activity or expression level of said polypeptide compared to the selected non-human organism or the part thereof;
(e) optionally, growing and cultivating the non-human organisms or the parts thereof;
and
(f) recovering, and optionally isolating, the free or bound linoleic acid produced by the selected mutated non-human organisms or parts thereof.

Item 5. The process of any one of items 1 to 4 wherein the activity of said protein or the expression of said nucleic acid molecule is increased or generated transiently or stably.

Item 6. An isolated nucleic acid molecule comprising a nucleic acid molecule selected from the group consisting of:
(a) a nucleic acid molecule encoding a polypeptide shown in Table II, application no. 8, column 5 or 8, preferably shown in Table II B, application no. 8, column 8, or a homolog or a fragment thereof;
(b) a nucleic acid molecule shown in Table I, application no. 8, column 5 or 8, preferably shown in Table I B, application no. 8, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 8, preferably in column 8 of Table II B, application no. application no.;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 3, preferably shown in column 8 of Table I B, application no. 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 8;
(i) a nucleic acid molecule encoding a polypeptide having the activity represented by a respective protein as depicted in column 5 of Table II, application no. 8;
(j) a nucleic acid molecule comprising a polynucleotide which is obtained by amplifying a cDNA library or a genomic library using the primers in Table III, application no. 8, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j); or a nucleic acid molecule comprising a sequence which is complementary thereto.

Item 7. A nucleic acid construct which confers the expression of the nucleic acid molecule as defined in item 6, comprising one or more regulatory elements.

Item 8. An expression cassette comprising
(a) a promoter, preferably selected from the group consisting of Big35S, PCUbi, Super and USP;
(b) a nucleic acid molecule as defined in item 6.

Item 9. A vector comprising the nucleic acid molecule as defined in item 6 or the nucleic acid construct as claimed in item 7 or the expression cassette as claimed in item 8.

Item 10. A host cell or a plant organelle, which has been transformed with the vector as claimed in item 9 or the nucleic acid molecule as defined in item 6 or the nucleic acid construct as claimed in item 7 or the expression cassette as claimed in item 8.

Item 11. The host cell of item 10, which is a plant cell or a microorganism.

Item 12. A process for producing a polypeptide, wherein the polypeptide is expressed in a host cell as claimed in item 10 or 11.

Item 13. A polypeptide produced by the process as claimed in item 12 or encoded by the nucleic acid molecule as defined in item 6.

Item 14. An antibody, which binds specifically to the polypeptide as claimed in item 13.

Item 15. A plant tissue, propagation material, harvested material or a plant comprising the host cell as claimed in item 10 or 11.

Item 16. A process for the identification of a compound conferring an increase in linoleic acid production in a non-human organism, comprising the steps:
(a) culturing a plant cell or tissue or microorganism or maintaining a plant expressing the polypeptide encoded by the nucleic acid molecule as defined in item 6 conferring an increase in the amount of linoleic acid in a non-human organism or a part thereof and a readout system capable of interacting with the polypeptide under suitable conditions which permit the interaction of the polypeptide with said readout system in the presence of a compound or a sample comprising a plurality of compounds and capable of providing a detectable signal in response to the binding of a compound to said polypeptide under conditions which permit the expression of said readout system and of the polypeptide encoded by the nucleic acid molecule of item 6 conferring an increase in the amount of linoleic acidin a non-human organism or a part thereof;
(b) identifying if the compound is an effective agonist by detecting the presence or absence or increase of a signal produced by said readout system.

Item 17. A method for the production of an agricultural composition comprising the steps of the method of item 16 and formulating the compound identified in item 16 in a form acceptable for an application in agriculture.

Item 18. A composition comprising the nucleic acid molecule as defined in item 6, the polypeptide as claimed in item 13, the nucleic acid construct as claimed in item 7, the expression cassette as claimed in item 8, the vector as claimed in item 9, the compound as claimed in item 16, the antibody as claimed in item 14, and optionally an agricultural acceptable carrier.

Item 19. Use of the nucleic acid molecule as defined in item 6 for the identification of a nucleic acid molecule conferring an increase in linoleic acid after expression.

Item 20. Cosmetic, pharmaceutical, food or feed composition comprising the nucleic acid molecule as defined in item 6, the polypeptide as claimed in item 13, the nucleic acid construct as claimed in item 7, the expression cassette as claimed in item 8, the vector as claimed in item 9, the antibody as claimed in item 14, the plant or a part thereof, or plant tissue as claimed in item 15, the harvested material or propagation material as claimed in item 15 or the host cell of item as claimed in items 10 or 11.

Item 21. Use of the nuclei acid molecule as defined in item 6, the polypeptide as claimed in item 13, the nucleic acid construct as claimed in item 7, the expression cassette as claimed in item 8, the vector as claimed in claim9, the plant or plant tissue as claimed in item 15, or the host cell of item 10 to 11 for the production of plant resistant to a herbicide inhibiting the production of linoleic acid.

Item 22. A method for producing a plant with increased yield as compared to a corresponding non-transformed wild type plant, whereby the method comprises
(i) increasing or generating of the expression of; and/or
(ii) increasing or generating the expression of an expression product of; and/or
(iii) increasing or generating one or more activities of an expression product encoded by;
at least one respective nucleic acid molecule comprising a nucleic acid molecule selected from the group consisting of:
(a) a nucleic acid molecule encoding the polypeptide shown in column 5 or 8 of Table II, preferably Table II B, application no. 8, or a homolog or a fragment thereof, in case in column 7 linoleic acid is indicated;
(b) a nucleic acid molecule shown in column 5 or 8 of Table I, preferably Table I B, application no. 8, or a homolog or a fragment thereof (preferably the coding region thereof) , in case in column 7 linoleic acid is indicated;
(c) a nucleic acid molecule, which, as a result of the degeneracy of the genetic code, can be derived from a polypeptide sequence depicted in column 5 or 8 of Table II, preferably Table II B, application no. 8, in case in column 7 linoleic acidis indicated;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 8, or the coding region thereof, in case in column 7 linoleic acid is indicated;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 8, in case in column 7 linoleic acid is indicated;
(i) a nucleic acid molecule encoding a polypeptide having the activity represented by a respective protein as depicted in column 5 of Table II, application no. 8, in case in column 7 linoleic acid is indicated;
(j) a nucleic acid molecule which comprises a polynucleotide, which is obtained by amplifying a cDNA library or a genomic library using the primers as depicted in column 8 of Table III, application no. 8, in case in column 7 linoleic acid is indicated; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto; in a plant cell, plant or a part thereof.

Item 23. A method for producing a plant with increased yield according to item 22, which comprises in addition the regeneration of a plant from such transformed plant cell, plant cell compartment, plant cell nucleus, plant cell or plant tissue with increased yield.

### EXAMPLES

### Example 1

### Cloning of the Sequences as Shown in Table I, Column 5 or 8 in Escherichia Coli

The inventive sequences as shown in the respective line in Table I, column 5 or 8 were cloned into the plasmids pBR322 (Sutcliffe J.G., Proc. Natl. Acad. Sci. USA, 75, 3737 (1979)), pA-CYC177 (Change and Cohen, J. Bacteriol. 134, 1141 (1978)), plasmids of the pBS series (pBSSK+, pBSSK− and others; Stratagene, LaJolla, USA) or cosmids such as SuperCosi (Stratagene, LaJolla, USA) or Lorist6 (Gibson T. J., Rosenthal A. and Waterson R. H., Gene 53, 283 (1987) for expression in E. coli using known, well-established procedures (see, for example, J. Sambrook et al. “Molecular Cloning: A Laboratory Manual”. Cold Spring Harbor Laboratory Press (1989) or F. M. Ausubel et al., “Current Protocols in Molecular Biology”, John Wiley & Sons (1994)).

### Example 2

### DNA Sequencing and Computerized Functional Analysis

The DNA was sequenced by standard procedures, in particular the chain determination method, using AB1377 sequencers (see, for example, Fleischman R. D. et al., Science 269, 496 (1995)).

### Example 3

### In-Vivo and In-Vitro Mutagenesis

An in vivo mutagenesis of organisms such as Saccharomyces, Mortierella, Escherichia and others mentioned above, which are beneficial for the production of a fine chemical can be carried out by passing a plasmid DNA (or another vector DNA) containing the desired nucleic acid sequence or nucleic acid sequences through E. coli and other microorganisms (for example Bacillus spp. or yeasts such as Saccharomyces cerevisiae) which are not capable of maintaining the integrity of its genetic information. Usual mutator strains have mutations in the genes for the DNA repair system (for example mutHLS, mutD, mutT and the like; for comparison, see Rupp W.D., “DNA repair mechanisms in Escherichia coli and Salmonella”, pp. 2277-2294, ASM, Washington (1996)]. The skilled worker knows these strains. The use of these strains is illustrated for example in Greener A., and Callahan M., Strategies 7, 32 (1994).

In-vitro mutation methods such as increasing the spontaneous mutation rates by chemical or physical treatment are well known to the skilled person. Mutagens like 5-bromo-uracil, N-methyl-N-nitro-N-nitrosoguanidine (=NTG), ethyl methanesulfonate (=EMS), hydroxylamine and/or nitrous acid are widely used as chemical agents for random in-vitro mutagenesis. The most common physical method for mutagenesis is the treatment with UV irradiation. Another random mutagenesis technique is the error-prone PCR for introducing amino acid changes into proteins. Mutations are deliberately introduced during PCR through the use of error-prone DNA polymerases and special reaction conditions known to a person skilled in the art. For this method randomized DNA sequences are cloned into expression vectors and the resulting mutant libraries screened for altered or improved protein activity as described below.

Site-directed mutagenesis method such as the introduction of desired mutations with an M13 or phagemid vector and short oligonucleotides primers is a well-known approach for site-directed mutagenesis. The clou of this method involves cloning of the nucleic acid sequence of the invention into an M13 or phagemid vector, which permits recovery of single-stranded recombinant nucleic acid sequence. A mutagenic oligonucleotide primer is then designed whose sequence is perfectly complementary to nucleic acid sequence in the region to be mutated, but with a single difference: at the intended mutation site it bears a base that is complementary to the desired mutant nucleotide rather than the original. The mutagenic oligonucleotide is then allowed to prime new DNA synthesis to create a complementary full-length sequence containing the desired mutation. Another site-directed mutagenesis method is the PCR mismatch primer mutagenesis method also known to the skilled person. Dpnl site-directed mutagenesis is a further known method as described for example in the Stratagene Quickchange™ site-directed mutagenesis kit protocol. A huge number of other methods are also known and used in common practice.

Positive mutation events can be selected by screening the organisms for the production of the desired fine chemical.

### Example 4

### DNA Transfer between Escherichia Coli, Saccharomyces Cerevisiae and Mortierella Alpina

Shuttle vectors such as pYE22m, pPAC-ResQ, pClasper, pAUR224, pAMH10, pAML10, pAMT10, pAMU10, pGMH10, pGML10, pGMT10, pGMU10, pPGAL1, pPADH1, pTADH1, pTAex3, pNGA142, pHT3101 and derivatives thereof which allow the transfer of nucleic acid sequences between Escherichia coli, Saccharomyces cerevisiae and/or Mortierella alpina are available to the skilled worker. An easy method to isolate such shuttle vectors is disclosed by Soni R. and Murray J. A. H., Nucleic Acid Research, 20 (21), 5852 (1992). If necessary such shuttle vectors can be constructed easily using standard vectors for E. coli (Sambrook J. et al. “Molecular Cloning: A Laboratory Manual”, Cold Spring Harbor Laboratory Press (1989) or Ausubel F. M. et al. “Current Protocols in Molecular Biology”, John Wiley & Sons (1994)) and/or the aforementioned vectors, which have a replication origin for, and suitable marker from, Escherichia coli, Saccharomyces cerevisiae or Mortierella alpina added. Such replication origins are preferably taken from endogenous plasmids, which have been isolated from species used in the inventive process. Genes, which are used in particular as transformation markers for these species are genes for kanamycin resistance (such as those which originate from the Tn5 or Tn903 transposon) or for chloramphenicol resistance (Winnacker E. L., “From Genes to Clones—Introduction to Gene Technology”, VCH, Weinheim (1987)) or for other antibiotic resistance genes such as for G418, gentamycin, neomycin, hygromycin or tetracycline resistance.

Using standard methods, it is possible to clone a gene of interest into one of the above-described shuttle vectors and to introduce such hybrid vectors into the microorganism strains used in the inventive process. The transformation of Saccharomyces can be achieved for example by LiCl or sheroplast transformation (Bishop et al., Mol. Cell. Biol., 6, 3401 (1986), Sherman et al., “Methods in Yeasts in Genetics”, Cold Spring Harbor Lab. Cold Spring Harbor, N.Y. (1982), Agatep et al., Technical Tips Online 1998, 1:51: P01525 or Gietz et al., Methods Mol. Cell. Biol. 5,255 (1995)) or electroporation (Delorme E., Appl. Environ. Microbiol., 55 (9), 2242 (1989)).

If the transformed sequence(s) is/are to be integrated advantageously into the genome of the microorganism used in the inventive process for example into the yeast or fungi genome, standard techniques known to the skilled worker also exist for this purpose. Solinger et al. (Proc. Natl. Acad. Sci. U S A., 15, 8447 (2001)) and Freedman et al. (Genetics, 162, 15 (2002)) teaches a homolog recombination system dependent on rad 50, rad51, rad54 and rad59 in yeasts. Vectors using this system for homologous recombination are vectors derived from the Ylp series. Plasmid vectors derived for example from the 2p-Vector are known by the skilled worker and used for the expression in yeasts. Other preferred vectors are for example pART1, pCHY21 or pEVP11 as they have been described by McLeod et al. (EMBO J., 6, 729 (1987)) and Hoffman et al. (Genes Dev. 5, 561 (1991)) or Russell et al. (J. Biol. Chem. 258, 143 (1983)). Other beneficial yeast vectors are plasmids of the REP, REP-X, pYZ or RIP series.

### Example 5

### Determining the Expression of the Mutant/Transgenic Protein

The observations of the activity of a mutated, or transgenic, protein in a transformed host cell are based on the fact that the protein is expressed in a similar manner and in a similar quantity as the wild-type protein. A suitable method for determining the transcription quantity of the mutant, or transgenic, gene (a sign for the amount of mRNA which is available for the translation of the gene product) is to carry out a Northern blot (see, for example, Ausubel et al., “Current Protocols in Molecular Biology”, Wiley, New York (1988)), where a primer which is designed in such a way that it binds to the gene of interest is provided with a detectable marker (usually a radioactive or chemiluminescent marker) so that, when the total RNA of a culture of the organism is extracted, separated on a gel, applied to a stable matrix and incubated with this probe, the binding and quantity of the binding of the probe indicates the presence and also the amount of mRNA for this gene. Another method is a quantitative PCR. This information detects the extent to which the gene has been transcribed. Total cell RNA can be isolated for example from yeasts or E. coli by a variety of methods, which are known in the art, for example with the Ambion kit according to the instructions of the manufacturer or as described in Edgington et al., Promega Notes Magazine Number 41, 14 (1993).

Standard techniques, such as Western blot, may be employed to determine the presence or relative amount of protein translated from this mRNA (see, for example, Ausubel et al. “Current Protocols in Molecular Biology”, Wiley, New York (1988)). In this method, total cell proteins are extracted, separated by gel electrophoresis, transferred to a matrix such as nitrocellulose and incubated with a probe, such as an antibody, which binds specifically to the desired protein.

This probe is usually provided directly or indirectly with a chemiluminescent or colorimetric marker, which can be detected readily. The presence and the observed amount of marker indicate the presence and the amount of the sought mutant protein in the cell. However, other methods are also known.

### Example 6

### Growth of Genetically Modified Organism: Media and Culture Conditions

Genetically modified Yeast, Mortierella or Escherichia coli are grown in synthetic or natural growth media known by the skilled worker. A number of different growth media for Yeast, Mortierella or Escherichia coli are well known and widely available. A method for culturing Mortierella is disclosed by Jang et al. (Bot. Bull. Acad. Sin. 41, 41 (2000)). Mortierella can be grown at 20° C. in a culture medium containing: 10 g/l glucose, 5 g/l yeast extract at pH 6.5. Furthermore Jang et al. teaches a submerged basal medium containing 20 g/l soluble starch, 5 g/l Bacto yeast extract, 10 g/l KNO₃, 1 g/l KH₂PO₄, and 0.5 g/l MgSO₄x7H₂O, pH 6.5.

Said media, which can be used according to the invention usually consist of one or more carbon sources, nitrogen sources, inorganic salts, vitamins and trace elements. Preferred carbon sources are sugars such as mono-, di- or polysaccharides. Examples of very good carbon sources are glucose, fructose, mannose, galactose, ribose, sorbose, ribulose, lactose, maltose, sucrose, raffinose, starch or cellulose. Sugars may also be added to the media via complex compounds such as molasses or other by-products of sugar refining. It may also be advantageous to add mixtures of various carbon sources. Other possible carbon sources are alcohols and/or organic acids such as methanol, ethanol, acetic acid or lactic acid. Nitrogen sources are usually organic or inorganic nitrogen compounds or materials containing said compounds. Examples of nitrogen sources include ammonia gas, aqueous ammonia solutions or ammonium salts such as NH₄Cl, or (NH₄)₂SO₄, NH₄OH, nitrates, urea, amino acids or complex nitrogen sources such as cornsteep liquor, soybean flour, soybean protein, yeast extract, meat extract and others. Mixtures of the above nitrogen sources may be used advantageously.

Inorganic salt compounds, which may be included in the media comprise the chloride, phosphorus or sulfate salts of calcium, magnesium, sodium, cobalt, molybdenum, potassium, manganese, zinc, copper and iron. Chelating agents may be added to the medium in order to keep the metal ions in solution. Particularly suitable chelating agents include dihydroxyphenols such as catechol or protocatechulate or organic acids such as citric acid. The media usually also contain other growth factors such as vitamins or growth promoters, which include, for example, biotin, riboflavin, thiamine, folic acid, nicotinic acid, panthothenate and pyridoxine. Growth factors and salts are frequently derived from complex media components such as yeast extract, molasses, cornsteep liquor and the like. The exact composition of the compounds used in the media depends heavily on the particular experiment and is decided upon individually for each specific case. Information on the optimization of media can be found in the textbook “Applied Microbiol. Physiology, A Practical Approach” (eds. Rhodes P. M., Stanbury P. F., IRL Press pp. 53-73 (1997)). Growth media can also be obtained from commercial suppliers, for example Standard 1 (Merck) or BHI (Brain heart infusion, DIFCO) and the like.

All media components are sterilized, either by heat (20 min at 1.5 bar and 121° C.) or by filter sterilization. The components may be sterilized either together or, if required, separately. All media components may be present at the start of the cultivation or added continuously or batchwise, as desired.

The culture conditions are defined separately for each experiment. The temperature is normally between 15° C. and 45° C. and may be kept constant or may be altered during the experiment. The pH of the medium should be in the range from 5 to 8.5, preferably around 7.0, and can be maintained by adding buffers to the media. An example of a buffer for this purpose is a potassium phosphate buffer. Synthetic buffers such as MOPS, HEPES, ACES and the like may be used as an alternative or simultaneously. The culture pH value may also be kept constant during the culture period by addition of, for example, NaOH or NH₄OH. If complex media components such as yeast extract are used, additional buffers are required less since many complex compounds have a high buffer capacity. When using a fermenter for the culture of microorganisms, the pH value can also be regulated using gaseous ammonia.

The incubation period is generally in a range of from several hours to several days. This time period is selected in such a way that the maximum amount of product accumulates in the fermentation broth. The growth experiments, which are disclosed, can be carried out in a multiplicity of containers such as microtiter plates, glass tubes, glass flasks or glass or metal fermenters of various sizes. To screen a large number of clones, the microorganisms should be grown in microtiter plates, glass tubes or shake flasks, either using simple flasks or baffle flasks. 100 ml shake flasks filled with 10% (based on the volume) of the growth medium required are preferably used. The flasks should be shaken on an orbital shaker (amplitude 25 mm) at a rate ranging from 100 to 300 rpm. Evaporation losses can be reduced by maintaining a humid atmosphere; as an alternative, a mathematical correction should be carried out for the evaporation losses.

If genetically modified clones are examined, an unmodified control clone, or a control clone, which contains the basic plasmid without insertion, should also be included in the tests. If a transgenic sequence is expressed, a control clone should advantageously again be included in these tests. The medium is advantageously inoculated to an OD600 of 0.5 to 1.5 using cells which have been grown on agar plates, such as CM plates (10 g/l glucose, 2.5 g/l NaCl, 2 g/l urea, 10 g/l polypeptone, 5 g/l yeast extract, 5 g/l meat extract, 22 g/l agar, pH value 6.8 established with 2M NaOH), which have been incubated at 30° C. The media are inoculated for example by addition of a liquid preculture of seed organism such as E. coli or S. cerevisiae.

### Example 7

### Growth of Genetically Modified Algae: Media and Culture Conditions

### Growing Chlamydomonas:

Chlamydomonas reinhardtii is able to grow under various growth conditions. It is a unicellular alga. The cells of Chlamydomonas reinhardtii can be normally cultured autotrophically in the media mentioned below. Cells of Chlamydomonas reinhardtii can be cultivated at 25° C. under cool-white fluorescence light at 10,000 lux (120 μmol m⁻²s⁻¹ photosynthetically active radiation) as described by Ghirardi et al., Appl. Biochem. Biotechnol. 63, 141 (1997) or Semin et al., Plant. Physiol., 131, 1756 (2003).

### Chlamydomonas Growth Medium:

1 l growth medium is prepared by adding the following volumes of the stock solutions as mentioned below:

1 ml solution A, 5 ml solution B, 1 ml solution C, 1 ml solution D, 3 ml solution E, 3 ml solution F, 1 ml solution G, 1 ml solution H.

| | |
|---|---|
| A) Trace elements solution: | 1 g/l H₃BO₃ |
| | 1 g/l ZnSO₄ × 7 H₂O |
| | 0.3 g/l MnSO₄ × H₂O |
| | 0.2 g/l CoCl₂ × 6 H₂O |
| | 0.2 g/l Na₂MoO₄ × 2 H₂O |
| | 0.04 g/l CuSO₄ |
| B) Na Citrate solution: | 10% w/v Na citrate × 2 H₂O |
| C) Iron solution: | 1% w/v FeCl₃ × 6 H₂O |
| D) Calcium solution: | 5.3% w/v CaCl₂ × H₂O |
| E) Magnesium solution: | 10% w/v MgSO₄ × 7 H₂O |
| F) Ammonium solution: | 10% w/v NH₄NO₃ |
| G) Potassium solution: | 10% w/v KH₂PO₄ |
| H) Dipotassium solution | 10% w/v K₂HPO₄ |

### Bristol's Soil Extract Medium:

Soil extract medium can generally be used for the growth of axenic and xenic algae cultures. The soil extract is prepared by adding a teaspoon of dry garden soil and a pinch of CaCO₃ to 200 ml distilled water and steaming said solution for approximately 2 h on two consecutive days. Afterwards the supernatant is decanted and added to the desired medium. To 940 ml bristol's solution 40 ml of soil extract medium is added.

### Bristol's Solution:

To 940 ml of distilled water, the following stock solutions are added:

| | | |
|---|---|---|
| 10 ml | NaNO₃ | (25 g/l) |
| 10 ml | CaCl₂ × 2 H₂O | (2.5 g/l) |
| 10 ml | MgSO₄ × 7 H₂O | (7.5 g/l) |
| 10 ml | K₂HPO₄ | (7.5 g/l) |
| 10 ml | KH₂PO₄ | (17.5 g/l) |
| 10 ml | NaCl | (2.5 g/l) |

Amplification and Cloning of DNA from Chlamydomonas Reinhardtii:

The DNA can be amplified by the polymerase chain reaction (PCR) from Chlamydomonas reinhardtii by the method of Howitt Crispin A. (BioTechniques 21, 32 (1996)).

### Fine Chemical Production in Chlamydomonas Reinhardtii:

The fine chemical production can be analysed as mentioned above. The proteins and nucleic acids can be analyzed as mentioned below.

### Example 8

### In-Vitro Analysis of the Function of the Proteins Encoded by the Transformed Sequences

The determination of the activities and kinetic parameters of enzymes is well known in the art. Experiments for determining the activity of a specific modified enzyme must be adapted to the specific activity of the wild-enzyme type, which is well within the capabilities of the skilled worker. Overviews of enzymes in general and specific details regarding the structure, kinetics, principles, methods, applications and examples for the determination of many enzyme activities can be found for example in the following literature: Dixon M. and Webb E. C.: “Enzymes”, Longmans, London (1979); Fersht “Enzyme Structure and Mechanism”, Freeman, New York (1985); Walsh “Enzymatic Reaction Mechanisms” Freeman, San Francisco (1979); Price N. C., Stevens L., “Fundamentals of Enzymology” Oxford Univ. Press, Oxford (1982); Boyer P. D. (ed.), “The Enzymes”, 3rd ed. Academic Press, New York (1983); Bisswanger H., “Enzymkinetik”, 2^{nd} ed. VCH, Weinheim (1994); Bergmeyer H. U., Bergmeyer J., GraRl M., (eds.) “Methods of Enzymatic Analysis”, 3rd rd. Vol. I-XII, Verlag Chemie: Weinheim (1983-1986); and “Ullmann's Encyclopedia of Industrial Chemistry” Vol. A9, “Enzymes”, VCH, Weinheim, pp. 352-363 (1987).

### Example 9

### Analysis of the Effect of the Nucleic Acid Molecule on the Production of the Fine Chemical

The effect of the genetic modification in plants, fungi, algae, ciliates on the production of a fine chemical can be determined by growing the modified microorganisms for example Chlamydomonas reinhardtii under suitable conditions (such as those described above) and analyzing the medium and/or the cellular components for the increased production of the fine chemical. Such analytical techniques are well known to the skilled worker and encompass spectroscopy, thin-layer chromatography, various types of staining methods, enzymatic and microbiological methods and analytical chromatography such as high-performance liquid chromatography (see, for example, “Ullmann Encyclopedia of Industrial Chemistry”, Vol. A2, pp. 89-90 and pp. 443-613, VCH, Weinheim (1985); Fallon A.et al., “Applications of HPLC in Biochemistry” in “Laboratory Techniques in Biochemistry and Molecular Biology”, Vol. 17 (1987); Rehm et al. “Biotechnology”, Vol. 3, Chapter III: “Product recovery and purification”, pp. 469-714, VCH: Weinheim (1993); Better P.A. et al. “Bioseparations: downstream processing for Biotechnology”, John Wiley and Sons (1988); Kennedy J.F. and Cabral J.M.S., “Recovery processes for biological Materials”, John Wiley and Sons(1992); Shaeiwitzand J. A., Henry J. D., “Biochemical Separations” in “Ullmann's Encyclopedia of Industrial Chemistry”, Vol. B3; chapter 11, pp. 1-27, VCH, Weinheim (1988); and Dechow, “Separation and purification techniques in biotechnology”, Noyes Publications (1989)).

In addition to the determination of the fermentation end product, other components of the metabolic pathways which are used for the production of the desired compound, such as intermediates and by-products, may also be analyzed in order to determine the total productivity of the organism, the yield and/or production efficiency of the compound. The analytical methods encompass determining the amounts of nutrients in the medium (for example sugars, hydrocarbons, nitrogen sources, phosphate and other ions), determining biomass composition and growth, analyzing the production of ordinary metabolites from biosynthetic pathways and measuring gases generated during the fermentation. Standard methods for these are described in “Applied Microbial Physiology” A Practical Approach, Rhodes P. M. and Stanbury P. F., eds. IRL Press, pp. 103-129; 131-163 and 165-192, and the references cited therein.

### Example 10

### Purification of a Fine Chemical e.g. Fatty Acids

The microorganism can be disrupted by sonication, grinding in a glass mill, liquid nitrogen and grinding, cooking, or via other applicable methods. After disruption centrifugation may follow. The sediment is resuspended in distilled water, heated for 10 minutes at 100° C., cooled on ice and recentrifuged, followed by extraction for one hour at 90° C. in 0.5 M sulfuric acid in methanol with 2% dimethoxypropane, which leads to hydrolyzed oil and lipid compounds, which give transmethylated lipids. These fatty acid methyl esters are extracted with petroleum ether and the solvent is evaporated lateron. (Analysis of the so obtained fatty acid ester(s) will be performed by GC analysis using a capillary column (Chrompack, WCOT Fused Silica, CP-Wax-52 CB, 25 micrometer, 0.32 mm) at a temperature gradient of between 170° C. and 240° C. for 20 minutes and 5 minutes at 240° C. The identity of the resulting fatty acid methyl esters can be determined using standards which are available from commercial sources (i.e. Sigma).)

### Example 11

### Cloning of the Sequences as Shown in Table I, Column 5 or 8 for the Expression in Plants

### Example 11a

### PCR Amplification of the Sequences

Unless otherwise specified, standard methods as described in Sambrook et al., Molecular Cloning: A laboratory manual, Cold Spring Harbor 1989, Cold Spring Harbor Laboratory Press are used.

The inventive sequences as shown in the respective line in Table I, column 5, preferably the coding region thereof, were amplified by PCR as described in the protocol of the Pfu Ultra, Pfu Turbo or Herculase DNA polymerase (Stratagene). The composition for the protocol of the Pfu Ultra, Pfu Turbo or Herculase DNA polymerase was as follows: 1×PCR buffer (Stratagene), 0.2 mM of each dNTP, 100 ng genomic DNA of Saccharomyces cerevisiae (strain S288C; Research Genetics, Inc., now Invitrogen), Escherichia coli (strain MG1655; E. coli Genetic Stock Center), Synechocystis sp. (strain PCC6803), Azotobacter vinelandii (strain N.R. Smith,16), Thermus thermophilus (HB8) or 50 ng cDNA from various tissues and development stages of Arabidopsis thaliana (ecotype Columbia), Physcomitrella patens, Glycine max (variety Resnick), Brassica napus, Oryza sativa or Zea mays (variety B73, Mo17, A188), 50 μmol forward primer, 50 μmol reverse primer, with or without 1 M Betaine, 2.5 u Pfu Ultra, Pfu Turbo or Herculase DNA polymerase.

The amplification cycles were as follows:

1 cycle of 2-3 minutes at 94-95° C., then 25-36 cycles with 30-60 seconds at 94-95° C., 30-45 seconds at 50-60° C. and 210-480 seconds at 72° C., followed by 1 cycle of 5-10 minutes at 72° C., then 4-16° C.—preferably for Saccharomyces cerevisiae, Escherichia coli, Synechocystis sp., Azotobacter vinelandii, Thermus thermophilus.

In case of Arabidopsis thaliana, Brassica napus, Glycine max, Oryza sativa, Physcomitrella patens, Zea mays the amplification cycles were as follows:

1 cycle with 30 seconds at 94° C., 30 seconds at 61° C., 15 minutes at 72° C., then 2 cycles with 30 seconds at 94° C., 30 seconds at 60° C., 15 minutes at 72° C., then 3 cycles with 30 seconds at 94° C., 30 seconds at 59° C., 15 minutes at 72° C., then 4 cycles with 30 seconds at 94° C., 30 seconds at 58° C., 15 minutes at 72° C., then 25 cycles with 30 seconds at 94° C., 30 seconds at 57° C., 15 minutes at 72° C., then 1 cycle with 10 minutes at 72° C., then finally 4-16° C. RNAs were generated with the RNeasy Plant Kit according to the standard protocol (Qiagen) and Superscript II Reverse Transkriptase was used to produce double stranded cDNA according to the standard protocol (Invitrogen).

ORF specific primer pairs for the genes to be expressed are shown in the respective line in Table III, column 8. The following adapter sequences were added to Saccharomyces cerevisiae ORF specific primers (see Table III) for cloning purposes:

SEQ ID NO: 20

| | | |
|---|---|---|
| i) | foward primer: | 5′-GGAATTCCAGCTGACCACC-3′ |

SEQ ID NO: 21

| | | |
|---|---|---|
| ii) | reverse primer: | 5′-GATCCCCGGGAATTGCCATG-3′ |

These adaptor sequences allow cloning of the ORF into the various vectors containing the Resgen adaptors, see table column E of Table c.

The following adapter sequences were added to Saccharomyces cerevisiae, Escherichia coli, Synechocystis sp., Azotobacter vinelandii, Thermus thermophilus, Arabidopsis thaliana, Brassica napus, Glycine max, Oryza sativa , Physcomitrella patens, or Zea mays ORF specific primers for cloning purposes:

| SEQ ID NO: 22 | | |
|---|---|---|
| iii) | forward primer: | 5′-TTGCTCTTCC- 3′ |
| SEQ ID NO: 23 | | |
| iiii) | reverse primer: | 5′-TTGCTCTTCG-3′ |

The adaptor sequences allow cloning of the ORF into the various vectors containing the Colic adaptors, see table column E of Table c.

Therefore for amplification and cloning of Saccharomyces cerevisiae SEQ ID NO: 66772, a primer consisting of the adaptor sequence i) and the ORF specific sequence SEQ ID NO: 67180 and a second primer consisting of the adaptor sequence ii) and the ORF specific sequence SEQ ID NO: 67181 were used.

For amplification and cloning of Escherichia coli SEQ ID NO: 7686, a primer consisting of the adaptor sequence iii) and the ORF specific sequence SEQ ID NO: 7726 and a second primer consisting of the adaptor sequence iiii) and the ORF specific sequence SEQ ID NO: 7727 were used.

For amplification and cloning of Synechocystis sp. SEQ ID NO: 11471, a primer consisting of the adaptor sequence iii) and the ORF specific sequence SEQ ID NO: 11983 and a second primer consisting of the adaptor sequence iiii) and the ORF specific sequence SEQ ID NO: 11984 were used.

For amplification and cloning of Arabidopsis thaliana SEQ ID NO: 1061, a primer consisting of the adaptor sequence iii) and the ORF specific sequence SEQ ID NO: 1293 and a second primer consisting of the adaptor sequence iiii) and the ORF specific sequence SEQ ID NO: 1294 were used.

For amplification and cloning of Azotobacter vinelandii SEQ ID NO: 6040, a primer consisting of the adaptor sequence iii) and the ORF specific sequence SEQ ID NO: 6070 and a second primer consisting of the adaptor sequence iiii) and the ORF specific sequence SEQ ID NO: 6071 were used.

For amplification and cloning of Glycine max SEQ ID NO: 16883, a primer consisting of the adaptor sequence iii) and the ORF specific sequence SEQ ID NO: 17345 and a second primer consisting of the adaptor sequence iiii) and the ORF specific sequence SEQ ID NO: 17346 were used.

For amplification and cloning of Thermus thermophilus SEQ ID NO: 61553, a primer consisting of the adaptor sequence iii) and the ORF specific sequence SEQ ID NO: 61719 and a second primer consisting of the adaptor sequence iiii) and the ORF specific sequence SEQ ID NO: 61720 were used.

Following these examples every sequence disclosed in Table I, preferably column 5, especially the coding region thereof can be cloned by fusing the adaptor sequences to the respective specific primers sequences as disclosed in Table III, column 8 using the respective vectors shown in Table c.

Table c showing binary vectors used in Example 11

TABLE c
Overview of the different vectors used for cloning the ORFs; showing their
SEQ ID NOs (column A), their vector names (column B), the promoters they
contain for expression of the ORFs (column C), if present, the additional artificial
targeting sequence (column D), the adapter sequence (column E), the expression
type conferred by the promoter mentioned in column B (column F)
and the figure number (column G).

| | B | C | D | E | | |
|---|---|---|---|---|---|---|
| A | Vector | Promoter | Target | Adapter | F | G |
| SeqID | Name | Name | Sequence | Sequence | Expression Type | FIG. |
| 30 | pMTX0270p | Super | | Colic | non targeted constitutive | 6 |
| | | | | | expression preferentially | |
| | | | | | in green tissues | |
| 31 | pMTX155 | Big35S | | Resgen | non targeted constitutive | 7 |
| | | | | | expression preferentially | |
| | | | | | in green tissues | |
| 32 | VC- | Super | FNR | Resgen | plastidic targeted constitutive | 3 |
| | MME354- | | | | expression preferentially | |
| | 1QCZ | | | | in green tissues | |
| 34 | VC- | Super | IVD | Resgen | mitochondric targeted | 8 |
| | MME356- | | | | constitutive expression | |
| | 1QCZ | | | | preferentially in green | |
| | | | | | tissues | |
| 36 | VC- | USP | | Resgen | non targeted expression | 9 |
| | MME301- | | | | preferentially in seeds | |
| | 1QCZ | | | | | |
| 37 | pMTX461korrp | USP | FNR | Resgen | plastidic targeted expression | 10 |
| | | | | | preferentially in | |
| | | | | | seeds | |
| 39 | VC- | USP | IVD | Resgen | mitochondric targeted | 11 |
| | MME462- | | | | expression preferentially | |
| | 1QCZ | | | | in seeds | |
| 41 | VC- | Super | | Colic | non targeted constitutive | 1 |
| | MME220- | | | | expression preferentially | |
| | 1qcz | | | | in green tissues | |
| 42 | VC- | Super | FNR | Colic | plastidic targeted constitutive | 4 |
| | MME432- | | | | expression preferentially | |
| | 1qcz | | | | in green tissues | |
| 44 | VC- | Super | IVD | Colic | mitochondric targeted | 12 |
| | MME431- | | | | constitutive expression | |
| | 1qcz | | | | preferentially in green | |
| | | | | | tissues | |
| 46 | VC- | PcUbi | | Colic | non targeted constitutive | 2 |
| | MME221- | | | | expression preferentially | |
| | 1qcz | | | | in green tissues | |
| 47 | pMTX447korr | PcUbi | FNR | Colic | plastidic targeted constitutive | 13 |
| | | | | | expression preferentially | |
| | | | | | in green tissues | |
| 49 | VC- | PcUbi | IVD | Colic | mitochondric targeted | 14 |
| | MME445- | | | | constitutive expression | |
| | 1qcz | | | | preferentially in green | |
| | | | | | tissues | |
| 51 | VC- | USP | | Colic | non targeted expression | 15 |
| | MME289- | | | | preferentially in seeds | |
| | 1qcz | | | | | |
| 52 | VC- | USP | FNR | Colic | plastidic targeted expression | 16 |
| | MME464- | | | | preferentially in | |
| | 1qcz | | | | seeds | |
| 54 | VC- | USP | IVD | Colic | mitochondric targeted | 17 |
| | MME465- | | | | expression in preferentially | |
| | 1qcz | | | | seeds | |
| 56 | VC- | Super | | Resgen | non targeted constitutive | 5 |
| | MME489- | | | | expression preferentially | |
| | 1QCZ | | | | in green tissues | |

In this Table c PcUbi refers to the PcUbi promoter (Kawalleck et al., Plant. Molecular Biology, 21, 673 (1993)) also being named p-PcUBI (e.g. in Table d), Super refers to the Super promoter (Ni et al,. Plant Journal 7, 661 (1995)) also being named p-Super (e.g. in Table d), Big35S refers to the enhanced 35S promoter (Comai et al., Plant Mol Biol 15, 373 (1990)) and USP to the USP promoter (Baumlein et al., Mol Gen Genet. 225(3), 459 (1991)) also being named p-USP in Table d.

### Example 11 b

### Construction of Binary Vectors for Non-Targeted Expression of Proteins

“Non-targeted” expression in this context means, that no additional targeting sequences were added to the ORF to be expressed.

For non-targeted expression the binary vectors used for cloning were VC-MME220-1 qcz SEQ ID NO 41 (FIG. 1), VC-MME221-1qcz SEQ ID NO 46 (FIG. 2), and VC-MME489-1QCZ SEQ ID NO: 56 (FIG. 5), respectively. The binary vectors used for cloning the targeting sequence were VC-MME489-1QCZ SEQ ID NO: 56 (FIG. 5) and pMTX0270p SEQ ID NO 30 (FIG. 6), respectively. Other useful binary vectors are known to the skilled worker; an overview of binary vectors and their use can be found in Hellens R., Mullineaux P. and Klee H. (Trends in Plant Science, 5 (10), 446 (2000)). Such vectors have to be equally equipped with appropriate promoters and targeting sequences.

### Example 11c

### Construction of Binary Vectors for Plastidic-Targeted Expression of Proteins

For construction of a vector for plastid-targeted expression in preferential green tissues or preferential in seeds, the plastidic targeting sequence of the gene FNR from Spinacia oleracea was amplified. In order to amplify the targeting sequence, genomic DNA was extracted from leaves of 4 weeks old S. oleracea plants (DNeasy Plant Mini Kit, Qiagen, Hilden). The gDNA was used as the template for a PCR.

To enable cloning of the transit sequence into the vector VC-MME489-1QCZ and VC-MME301-1QCZ an EcoRl restriction enzyme recognition sequence was added to both the forward and reverse primers, whereas for cloning in the vectors pMTX0270p, VC-MME220-1 qcz, VC-MME221-1qcz and VC-MME289-1qcz a Pmel restriction enzyme recognition sequence was added to the forward primer and a Ncol site was added to the reverse primer.

FNR5EcoResgen
SEQ ID NO: 24
ATA GAA TTC GCA TAA ACT TAT CTT CAT AGT TGC C
FNR3EcoResgen
SEQ ID NO: 25
ATA GAA TTC AGA GGC GAT CTG GGC CCT
FNR5PmeColic
SEQ ID NO: 26
ATA GTT TAA ACG CAT AAA CTT ATC TTC ATA GTT GCC
FNR3NcoColic
SEQ ID NO: 27
ATA CCA TGG AAG AGC AAG AGG CGA TCT GGG CCC T

The resulting sequence SEQ ID NO: 28 amplified from genomic spinach DNA, comprised a 5″UTR (bp 1-165), and the coding region (bp 166-273 and 351-419). The coding sequence is interrupted by an intronic sequence from by 274 to by 350:

(SEQ ID NO: 28)
gcataaacttatcttcatagttgccactccaatttgctccttgaatc
tcctccacccaatacataatccactcctccatcacccacttcactac
taaatcaaacttaactctgtttttctctctcctcctttcatttctta
ttcttccaatcatcgtactccgccatgaccaccgctgtcaccgccgc
tgtttctttcccctctaccaaaaccacctctctctccgcccgaagct
cctccgtcatttcccctgacaaaatcagctacaaaaaggtgattccc
aatttcactgtgttttttattaataatttgttattttgatgatgaga
tgattaatttgggtgctgcaggttcctttgtactacaggaatgtatc
tgcaactgggaaaatgggacccatcagggcccagatcgcctct

The PCR fragment derived with the primers FNR5EcoResgen and FNR3EcoResgen was digested with EcoRl and ligated in the vectors VC-MME489-1QCZ and VC-MME301-1QCZ, that had also been digested with EcoRl. The correct orientation of the FNR targeting sequence was tested by sequencing. The vector generated in this ligation step were VC-MME354-1 QCZ and pMTX461 korrp, respectively.

The PCR fragment derived with the primers FNR5PmeColic and FNR3NcoColic was digested with Pmel and Ncol and ligated in the vectors pMTX0270p, VC-MME220-1qcz, VC-MME221-1qcz and VC-MME289-1qcz that had been digested with Smal and Ncol. The vectors generated in this ligation step were VC-MME432-1qcz, VC-MME464-1qcz and pMTX447korr, respectively.

For plastidic-targeted constitutive expression in preferentially green tissues an artifical promoter A(ocs)3AmasPmas promoter (Super promoter)) (Ni et al, Plant Journal 7, 661 (1995), WO 95/14098) was used in context of the vector VC-MME354-1QCZ for ORFs from Saccharomyces cerevisiae as well as Arabidopsis thaliana and in context of the vector VC-MME432-1 qcz for ORFs from Escherichia coli as well as Arabidopsis thaliana, resulting in each case in an “inframe” fusion of the FNR targeting sequence with the ORFs.

For plastidic-targeted expression in preferentially seeds the USP promoter (Baumlein et al., Mol Gen Genet. 225(3):459-67 (1991)) was used in context of either the vector pMTX461 korrp for ORFs from Saccharomyces cerevisiae or in context of the vector VC-MME464-1 qcz for ORFs from Escherichia coli, resulting in each case in an “in-frame” fusion of the FNR targeting sequence with the ORFs.

For plastidic-targeted constitutive expression in preferentially green tissues and seeds the PcUbi promoter was used in context of the vector pMTX447korr for ORFs from Saccharomyces cerevisiae, Escherichia coli, Synechocystis sp., Azotobacter vinelandii, Thermus thermophilus, Arabidopsis thaliana, Brassica napus, Glycine max, Oryza sativa, Physcomitrella patens, or Zea mays, resulting in each case in an “in-frame” fusion of the FNR targeting sequence with the ORFs.

### Example 11d

### Construction of Binary Vectors for Mitochondric-Targeted Expression of Proteins

Amplification of the mitochondrial targeting sequence of the gene IVD from Arabidopsis thaliana and construction of vector for mitochondrial-targeted expression in preferential green tissues or preferential in seeds.

In order to amplify the targeting sequence of the IVD gene from A. thaliana, genomic DNA was extracted from leaves of A. thaliana plants (DNeasy Plant Mini Kit, Qiagen, Hilden). The gDNA was used as the template for a PCR.

To enable cloning of the transit sequence into the vectors VC-MME489-1 QCZ and VC-MME301-1QCZ an EcoRl restriction enzyme recognition sequence was added to both the for-ward and reverse primers, whereas for cloning in the vectors VC-MME220-1qcz, VC-MME221-1qcz and VC-MME289-1qcz a Pmel restriction enzyme recognition sequence was added to the forward primer and a Ncol site was added to the reverse primer.

IVD5EcoResgen
SEQ ID NO: 57
ATA GAA TTC ATG CAG AGG TTT TTC TCC GC
IVD3EcoResgen
SEQ ID NO: 58
ATA GAA TTC CGA AGA ACG AGA AGA GAA AG
IVD5PmeColic
SEQ ID NO: 59
ATA GTT TAA ACA TGC AGA GGT TTT TCT CCG C
IVD3NcoColic
SEQ ID NO: 60
ATA CCA TGG AAG AGC AAA GGA GAG ACG AAG AAC GAG

The resulting sequence (SEQ ID NO: 61) amplified from genomic A. thaliana DNA with IVD5EcoResgen and IVD3EcoResgen comprised 81 bp:

(SEQ ID NO: 61)
atgcagaggtttttctccgccagatcgattctcggttacgccgtca
agacgcggaggaggtctttctcttctcgttcttcg

The resulting sequence (SEQ ID NO: 62) amplified from genomic A. thaliana DNA with IVD5PmeColic and IVD3NcoColic comprised 89 bp:

(SEQ ID NO: 62)
atgcagaggtttttctccgccagatcgattctcggttacgccgtcaa
gacgcggaggaggtctttctcttctcgttcttcgtctctcct

The PCR fragment derived with the primers IVD5EcoResgen and IVD3EcoResgen was digested with EcoRl and ligated in the vectors VC-MME489-1QCZ and VC-MME301-1QCZ that had also been digested with EcoRl. The correct orientation of the IVD targeting sequence was tested by sequencing. The vectors generated in this ligation step were VC-MME356-1 QCZ and VC-MME462-1 QCZ, respectively.

The PCR fragment derived with the primers IVD5PmeColic and IVD3NcoColic was digested with Pmel and Ncol and ligated in the vectors VC-MME220-1qcz, VC-MME221-1qcz and VCMME289-1qcz that had been digested with Smal and Ncol. The vectors generated in this ligation step were VC-MME431-1qcz, VC-MME465-1qcz and VC-MME445-1qcz, respectively.

For mitochondrial-targeted constitutive expression in preferentially green tissues an artificial promoter A(ocs)3AmasPmas promoter (Super promoter) (Ni et al, Plant Journal 7, 661 (1995), WO 95/14098) was used in context of the vector VC-MME356-1 QCZ for ORFs from Saccharomyces cerevisiae and in context of the vector VC-MME431-1qcz for ORFs from Escherichia coli, resulting in each case in an “in-frame” fusion between the IVD sequence and the respective ORFs.

For mitochondrial-targeted constitutive expression in preferentially seeds the USP promoter (Bäumlein et al., Mol Gen Genet. 225 (3), 459 (1991)) was used in context of the vector VC-MME462-1 QCZ for ORFs from Saccharomyces cerevisiae and in context of the vector VC-MME465-1qcz for ORFs from Escherichia coli, resulting in each case in an “in-frame” fusion between the IVD sequence and the respective ORFs.

For mitochondrial-targeted constitutive expression in preferentially green tissues and seeds the PcUbi promoter was used in context of the vector VC-MME445-1 qcz for ORFs from Saccharomyces cerevisiae, Escherichia coli, Synechocystis sp., Azotobacter vinelandii, Thermus thermophilus, Arabidopsis thaliana, Brassica napus, Glycine max, Oryza sativa, Physcomitrella patens, or Zea mays, resulting in each case in an “in-frame” fusion between the IVD sequence and the respective ORFs.

Other useful binary vectors are known to the skilled worker; an overview of binary vectors and their use can be found in Hellens R., Mullineaux P. and Klee H., (Trends in Plant Science, 5 (10), 446 (2000)). Such vectors have to be equally equipped with appropriate promoters and targeting sequences.

### Example 11e

### Cloning of Inventive Sequences as Shown in Table I, Column 5 in the Different Expression Vectors

For cloning of for example SEQ ID NO: 7686 from Escherichia coli or any other ORF from Escherichia coli, Synechocystis sp., Azotobacter vinelandii, Thermus thermophilus, Arabidopsis thaliana, or Glycine max the vector DNA was treated with the restriction enzymes Pacl and Ncol following the standard protocol (MBI Fermentas).

For cloning for example the ORFs of SEQ ID NO: 66772 from Saccharomyces cerevisiae or any other ORF from Saccharomyces cerevisiae into vectors containing the Resgen adaptor sequence the respective vector DNA was treated with the restriction enzyme Ncol.

For cloning of for example ORFs SEQ ID NO: 117352 from Saccharomyces cerevisiae or any other ORF from Saccharomyces cerevisiae into vectors containing the Colic adaptor sequence, the respective vector DNA was treated with the restriction enzymes Pacl and Ncol following the standard protocol (MBI Fermentas).

In all cases the reaction was stopped by inactivation at 70° C. for 20 minutes and purified over QlAquick or NucleoSpin Extract II columns following the standard protocol (Qiagen or Macherey-Nagel).

Then the PCR-product representing the amplified ORF with the respective adapter sequences and the vector DNA were treated with T4 DNA polymerase according to the standard protocol

(MBI Fermentas) to produce single stranded overhangs with the parameters 1 unit T4 DNA polymerase at 37° C. for 2-10 minutes for the vector and 1-2 u T4 DNA polymerase at 15-17° C. for 10-60 minutes for the PCR product representing SEQ ID NO: 7686.

The reaction was stopped by addition of high-salt buffer and purified over QlAquick or NucleoSpin Extract II columns following the standard protocol (Qiagen or Macherey-Nagel).

According to this example the skilled person is able to clone all sequences disclosed in Table I, preferably column 5 or column 8, especially the coding region thereof.

Approximately 30-60 ng of prepared vector and a defined amount of prepared amplificate were mixed and hybridized at 65° C. for 15 minutes followed by 37° C. 0.1° C/1 seconds, followed by 37° C. 10 minutes, followed by 0.1° C/1 seconds, then 4-10° C.

The ligated constructs were transformed in the same reaction vessel by addition of competent E. coli cells (strain DHSalpha) and incubation for 20 minutes at 1° C. followed by a heat shock for 90 seconds at 42° C. and cooling to 1-4° C. Then, complete medium (SOC) was added and the mixture was incubated for 45 minutes at 37° C. The entire mixture was subsequently plated onto an agar plate with 0.05 mg/ml kanamycin and incubated overnight at 37° C.

The outcome of the cloning step was verified by amplification with the aid of primers which bind upstream and downstream of the integration site, thus allowing the amplification of the insertion. The amplifications were carried out as described in the protocol of Taq DNA polymerase (Gibco-BRL).

The amplification cycles were as follows:

1 cycle of 1-5 minutes at 94° C., followed by 35 cycles of in each case 15-60 seconds at 94° C., 15-60 seconds at 50-66° C. and 5-15 minutes at 72° C., followed by 1 cycle of 10 minutes at 72° C., then 4-16° C.

Several colonies were checked, but only one colony for which a PCR product of the expected size was detected was used in the following steps.

A portion of this positive colony was transferred into a reaction vessel filled with complete medium (LB) supplemented with kanamycin and incubated overnight at 37° C.

The plasmid preparation was carried out as specified in the Qiaprep or NucleoSpin Multi-96 Plus standard protocol (Qiagen or Macherey-Nagel).

### Example 11f

### Generation of Transgenic Plants which Express Sequences as Shown in Table I, Preferably Column 5

1-5 ng of the plasmid DNA isolated was transformed by electroporation or transformation into competent cells of Agrobacterium tumefaciens, of strain GV 3101 pMP90 (Koncz and Schell, Mol. Gen. Gent. 204, 383 (1986)). Thereafter, complete medium (YEP) was added and the mixture was transferred into a fresh reaction vessel for 3 hours at 28° C. Thereafter, all of the reaction mixture was plated onto YEP agar plates supplemented with the respective antibiotics, e.g. rifampicine (0.1 mg/ml), gentamycine (0.025 mg/ml and kanamycin (0.05 mg/ml) and incubated for 48 hours at 28° C.

The agrobacteria that contains the plasmid construct were then used for the transformation of plants.

A colony was picked from the agar plate with the aid of a pipette tip and taken up in 3 ml of liquid TB medium, which also contained suitable antibiotics as described above. The preculture was grown for 48 hours at 28° C. and 120 rpm.

400 ml of LB medium containing the same antibiotics as above were used for the main culture. The preculture was transferred into the main culture. It was grown for 18 hours at 28° C. and 120 rpm. After centrifugation at 4 000 rpm, the pellet was resuspended in infiltration medium (MS medium, 10% sucrose).

In order to grow the plants for the transformation, dishes (Piki Saat 80, green, provided with a screen bottom, 30×20×4.5 cm, from Wiesauplast, Kunststofftechnik, Germany) were half-filled with a GS 90 substrate (standard soil, Werkverband E. V., Germany). The dishes were watered overnight with 0.05% Proplant solution (Chimac-Apriphar, Belgium). A. thaliana C24 seeds (Nottingham Arabidopsis Stock Centre, UK; NASC Stock N906) were scattered over the dish, approximately 1 000 seeds per dish. The dishes were covered with a hood and placed in the stratification facility (8 h, 110 μmol m⁻² s⁻¹, 22° C.; 16 h, dark, 6° C.). After 5 days, the dishes were placed into the short-day controlled environment chamber (8 h, 130 μmol m⁻2 s⁻1, 22° C.; 16 h, dark, 20° C.), where they remained for approximately 10 days until the first true leaves had formed.

The seedlings were transferred into pots containing the same substrate (Teku pots, 7 cm, LC series, manufactured by Pöppelmann GmbH & Co, Germany). Five plants were pricked out into each pot. The pots were then returned into the short-day controlled environment chamber for the plant to continue growing.

After 10 days, the plants were transferred into the greenhouse cabinet (supplementary illumination, 16 h, 340 μmol m⁻² s⁻¹, 22° C.; 8 h, dark, 20° C.), where they were allowed to grow for further 17 days.

For the transformation, 6-week-old Arabidopsis plants, which had just started flowering were immersed for 10 seconds into the above-described agrobacterial suspension which had previously been treated with 10 μl Silwett L77 (Crompton S. A., Osi Specialties, Switzerland). The method in question is described by Clough J.C.and Bent A. F. (Plant J. 16, 735 (1998)).

The plants were subsequently placed for 18 hours into a humid chamber. Thereafter, the pots were returned to the greenhouse for the plants to continue growing. The plants remained in the greenhouse for another 10 weeks until the seeds were ready for harvesting.

Depending on the tolerance marker used for the selection of the transformed plants the harvested seeds were planted in the greenhouse and subjected to a spray selection or else first sterilized and then grown on agar plates supplemented with the respective selection agent. Since the vector contained the bar gene as the tolerance marker, plantlets were sprayed four times at an interval of 2 to 3 days with 0.02% BASTA® and transformed plants were allowed to set seeds.

The seeds of the transgenic A. thaliana plants were stored in the freezer (at −20° C.).

### Example 11g

### Plant Culture (Arabidopsis) for Bioanalytical Analyses

For the bioanalytical analyses of the transgenic plants, the latter were grown uniformly a specific culture facility. To this end the GS-90 substrate as the compost mixture was introduced into the potting machine (Laible System GmbH, Singen, Germany) and filled into the pots. Thereafter, 35 pots were combined in one dish and treated with Previcur. For the treatment, 25 ml of Previcur were taken up in 10 I of tap water. This amount was sufficient for the treatment of approximately 200 pots. The pots were placed into the Previcur solution and additionally irrigated overhead with tap water without Previcur. They were used within four days.

For the sowing, the seeds, which had been stored in the refrigerator (at −20° C.), were removed from the Eppendorf tubes with the aid of a toothpick and transferred into the pots with the compost. In total, approximately 5 to 12 seeds were distributed in the middle of the pot.

After the seeds had been sown, the dishes with the pots were covered with matching plastic hood and placed into the stratification chamber for 4 days in the dark at 4° C. The humidity was approximately 90%. After the stratification, the test plants were grown for 22 to 23 days at a 16-h-light, 8-h-dark rhythm at 20° C., an atmospheric humidity of 60% and a CO₂ concentration of approximately 400 ppm. The light sources used were Powerstar HQI-T 250 W/D Daylight lamps from Osram, which generate a light resembling the solar color spectrum with a light intensity of approximately 220 E m²s⁻¹.

Selection of transgenic plants was depending on the use resistance marker. In case of the bar gene as the resistance marker plantlets were sprayed three times at days 8-10 after sowing with 0.02% BASTA® (Glufosinate ammonium; Bayer CropScience, Germany).The resistant plants were thinned when they had reached the age of 14 days. The plants, which had grown best in the center of the pot were considered the target plants. All the remaining plants were removed carefully with the aid of metal tweezers and discarded.

During their growth, the plants received overhead irrigation with distilled water (onto the compost) and bottom irrigation into the placement grooves. Once the grown plants had reached the age of 23 days, they were harvested. In case their seeds are desired these had been harvested 10 to 12 weeks after sowing (once they are ripe).

### Example 11h

### Metabolic Analysis of Transformed Plants

The modifications identified in accordance with the invention, in the content of above-described metabolites, were identified by the following procedure.

### a) Sampling and Storage of the Samples

Sampling was performed directly in the controlled-environment chamber. The plants, or respective parts thereof, like leafs, were cut using small laboratory scissors, rapidly weighed on laboratory scales, transferred into a pre-cooled extraction sleeve and placed into an aluminum rack cooled by liquid nitrogen. If required, the extraction sleeves can be stored in the freezer at −80° C. The time elapsing between cutting the plant/plant parts to freezing it in liquid nitrogen amounted to not more than 10 to 20 seconds.

### b) Lyophilization

During the experiment, care was taken that the plants either remained in the deep-frozen state (temperatures <−40° C.) or were freed from water by lyophilization until the first contact with solvents.

The aluminum rack with the plant samples in the extraction sleeves was placed into the precooled (−40° C.) lyophilization facility. The initial temperature during the main drying phase was −35° C. and the pressure was 0.120 mbar. During the drying phase, the parameters were altered following a pressure and temperature program. The final temperature after 12 hours was +30° C. and the final pressure was 0.001 to 0.004 mbar. After the vacuum pump and the refrigerating machine had been switched off, the system was flushed with air (dried via a drying tube) or argon.

### c) Extraction

### Extraction of Arabidopsis Green Tissue:

Immediately after the lyophilization apparatus had been flushed, the extraction sleeves with the lyophilized plant material were transferred into the 5 ml extraction cartridges of the ASE device (Accelerated Solvent Extractor ASE 200 with Solvent Controller and AutoASE software (DIONEX)).

The 24 sample positions of an ASE device (Accelerated Solvent Extractor ASE 200 with Solvent Controller and AutoASE software (DIONEX)) were filled with plant samples, including some samples for testing quality control.

The polar substances were extracted with approximately 10 ml of methanol/water (80/20, v/v) at T=70° C. and p=140 bar, 5 minutes heating-up phase, 1 minute static extraction. The more lipophilic substances were extracted with approximately 10 ml of methanol/dichloromethane (40/60, v/v) at T=70° C. and p=140 bar, 5 minute heating-up phase, 1 minute static extraction. The two solvent mixtures were extracted into the same glass tubes (centrifuge tubes, 50 ml, equipped with screw cap and pierceable septum for the ASE (DIONEX)).

The solution was treated with internal standards: ribitol, L-glycine-2,2-d2, L-alanine-2,3,3,3-d₄, methionine-methyl-d₃, and α-methylglucopyranoside and methyl nonadecanoate, methyl undecanoate, methyl tridecanoate, methyl pentadecanoate, methyl nonacosanoate.

The total extract was treated with 8 ml of water. The solid residue of the plant sample and the extraction sleeve were discarded.

The extract was shaken and then centrifuged for 5 to 10 minutes at at least 1400 g in order to accelerate phase separation. 1 ml of the supernatant methanol/water phase (“polar phase”, colorless) was removed for the further GC analysis, and 1 ml was removed for the LC analysis. The remainder of the methanol/water phase was discarded. 0.5 ml of the organic phase (“lipid phase”, dark green) was removed for the further GC analysis and 0.5 ml was removed for the LC analysis. All the portions removed were evaporated to dryness using the IR Dancer infrared vacuum evaporator (Hettich). The maximum temperature during the evaporation process did not exceed 40° C. Pressure in the apparatus was not less than 10 mbar.

### Extraction of Arabidopsis Seeds:

3 mg of Arabidopsis seeds are transferred into a 1.2-ml-stainless steel grinding jar and ground and extracted with a mixture of 770 μl methanol and 290 μl water. A solution containing commercially available standard substances (ribitol, L-glycine-2,2-d2, L-alanine-2,3,3,3-d4, methionine-methyl-d3, tryptophane-d5, Arginine 13C615N4, Pep3 (Boc-Ala-Gly-Gly-Gly-OH) and α-methylglucopyranoside) is added as internal standard. The extraction is performed using a stainless steel ball and a ball mill (Retsch MM 200, Retsch, Germany) operated at 30 Hz for 3 minutes. After centrifugation at 6000 rpm for 5 minutes 800 μl of the extraction solvent is transferred into a 2-ml-reaction tube (Eppendorf).

A solution of commercially available internal standard substances (Coenzyme Q1, Coenzyme Q2, Coenzyme Q4, and methyl nonadecanoate, undecanoic acid, tridecanoic acid, pentadecanoic acid, methyl nonacosanoate) is added to the residue as internal standard. For the extraction of lipophilic metabolites, 640 μl methylene chloride and 170 μl methanol are added and the sample is extracted in a ball mill operated at 30 Hz for 3 minutes. After centrifugation at 6000 rpm for 5 minutes 800 μl of the extraction solvent is transferred and combined with the extract of the first extraction step. After the addition of 400 μl of water and a centrifugation step to ensure proper separation of the organic and aqueous layer, two aliquots of 500 μl of the aqueous layer (polar phase) are taken for GC and LC analysis, respectively. Also two aliquots of 100 μl of the organic layer (lipid phase) are take for GC and LC analysis, respectively. All the portions removed were evaporated to dryness using the IR Dancer infrared vacuum evaporator (Hettich). The maximum temperature during the evaporation process did not exceed 40° C. Pressure in the apparatus was not less than 10 mbar.

### Extraction of Rice or Corn Seed Material:

20 rice or corn kernels are homogenized with a 50-ml-stainless steel grinding jar and ground with a stainless steel grinding ball using a ball mill (Retsch MM 200, Retsch, Germany) operated at 30 Hz for 3 minutes. The ground samples are lyophilized over night The initial temperature during the main drying phase was −35 ° C. and the pressure was 0.120 mbar. During the drying phase, the parameters were altered following a pressure and temperature program. The final temperature after 12 hours was +30° C. and the final pressure was 0.001 to 0.004 mbar. After the vacuum pump and the refrigerating machine had been switched off, the system was flushed with air (dried via a drying tube) or argon.

50 mg of the lyophilized kernel material are weighed into glass fibre extraction thimbles and extracted and further processed as described for the extraction of Arabidopsis green tissue.

### d) Processing the Lipid and Polar Phase for the LC/MS or LC/MS/MS Analysis

The lipid extract, which had been evaporated to dryness was taken up in mobile phase.

The polar extract, which had been evaporated to dryness was taken up in mobile phase.

### LC-MS Analysis:

The LC part was carried out on a commercially available LC/MS system from Agilent Technologies, USA. For polar extracts 10 μl are injected into the system at a flow rate of 200 μl/min. The separation column (Reversed Phase C18) was maintained at 15 ° C. during chromatography. For lipid extracts 5 μl are injected into the system at a flow rate of 200 μl/min. The separation column (Reversed Phase C18) was maintained at 30 ° C. HPLC was performed with gradient elution. The mass spectrometric analysis was performed on an Applied Biosystems API 4000 triple quadrupole instrument with turbo ion spray source. For polar extracts the instrument measured in negative ion mode in MRM-mode and fullscan mode from 100-1000 amu. For lipid extracts the instrument measured in positive ion mode in MRM-mode fullscan mode from 100-1000 amu. MS analysis is described in more detail in patent publication number WO 03/073464.

### e) Derivatization of the Lipid and Polar Phases for the GC/MS Analysis

### Derivatization of the Lipid Phase for the GC/MS Analysis:

For the transmethanolysis, a mixture of 140 μl of chloroform, 37 μl of hydrochloric acid (37% by weight HCl in water), 320 μl of methanol and 20 μl of toluene was added to the evaporated extract of the lipid phase . The vessel was sealed tightly and heated for 2 hours at 100° C., with shaking. The solution was subsequently evaporated to dryness. The residue was dried completely.

The methoximation of the carbonyl groups was carried out by reaction with methoxyamine hydrochloride (5 mg/ml in pyridine, 100 μl for 1.5 hours at 60° C.) in a tightly sealed vessel. 20 μl of a solution of odd-numbered, straight-chain fatty acids (solution of each 0.3 mg/ml of fatty acids from 7 to 25 carbon atoms and each 0.6 mg/ml of fatty acids with 27, 29 and 31 carbon atoms in 3/7 (v/v) pyridine/toluene) were added as time standards. Finally, the derivatization with 100 μl of N-methyl-N-(trimethylsilyl)-2,2,2-trifluoroacetamide (MSTFA) was carried out for 30 minutes at 60° C., again in the tightly sealed vessel. The final volume before injection into the GC was 220 μl.

### Derivatization of the Polar Phase for the GC/MS Analysis:

The methoximation of the carbonyl groups was carried out by reaction with methoxyamine hydrochloride (5 mg/ml in pyridine, 50 μl for 1.5 hours at 60° C.) in a tightly sealed vessel. 10 μl of a solution of odd-numbered, straight-chain fatty acids (solution of each 0.3 mg/ml of fatty acids from 7 to 25 carbon atoms and each 0.6 mg/ml of fatty acids with 27, 29 and 31 carbon atoms in 3/7 (v/v) pyridine/toluene) were added as time standards. Finally, the derivatization with 50 μl of N-methyl-N-(trimethylsilyl)-2,2,2-trifluoroacetamide (MSTFA) was carried out for 30 minutes at 60° C., again in the tightly sealed vessel. The final volume before injection into the GC was 110 μl.

### f) GC-MS Analysis

The GC-MS systems consisted of an Agilent 6890 GC coupled to an Agilent 5973 MSD. The autosamplers were CompiPal or GCPal from CTC. For the analysis usual commercial capillary separation columns (30 m×0.25 mm×0.25 pm) with different poly-methyl-siloxane stationary phases containing 0% up to 35% of aromatic moieties, depending on the analysed sample materials and fractions from the phase separation step, were used (for example: DB-1ms, HP-5ms, DB-XLB, DB-35ms, Agilent Technologies). Up to 1 μl of the final volume was injected splitless and the oven temperature program was started at 70 ° C. and ended at 340 ° C. with different heating rates depending on the sample material and fraction from the phase separation step in order to achieve a sufficient chromatographic separation and number of scans within each analyte peak. Usual GC-MS standard conditions, for example constant flow with nominal 1 to 1.7 ml/min. and helium as the mobile phase gas were used. Ionisation was done by electron impact with 70 eV, scanning within a m/z range from 15 to 600 with scan rates from 2.5 to 3 scans/sec and standard tune conditions.

### g) Analysis of the Various Plant Samples

The samples were measured in individual series of 20 to 21 plant or seed samples each (also referred to as sequences), each sequence containing at least 5 wild-type plants or seed samples as controls. Seed samples were from individual plants. The peak area of each analyte was divided by the peak area of the respective internal standard. The data were standardized for the fresh weight established for the plant or seed sample, respectively. The values calculated thus were related to the wild-type control group by being divided by the mean of the corresponding data of the wild-type control group of the same sequence. The values obtained were referred to as ratio_by_WT, they are comparable between sequences and indicate how much the analyte concentration in the transgenic plant sample differs in relation to the wild-type control. Appropriate controls were done before to proof that the vector and transformation procedure itself has no significant influence on the metabolic composition of the plants. Therefore the described changes in comparison with wild types were caused by the introduced gene constructs. At least 3-5 independent lines were analyzed in two independent experiments for each construct.

The results of the different plant analyses can be seen from the Table d.

Table d showing results of plant analyses

TABLE d

| SeqID | Target | Locus | Metabolite | Source | Promotor | Method | Min | Max |
|---|---|---|---|---|---|---|---|---|
| 113651 | non- | At1g19670 | linoleic | ARA_LEAF | p-PcUBI | GC | 15 | 24 |
| | targeted | | acid | | | | | |
| 17637 | non- | At1g19800 | linoleic | ARA_LEAF | p-PcUBI | GC | 14 | 29 |
| | targeted | | acid | | | | | |
| 1061 | non- | At1g68320 | linoleic | ARA_LEAF | p-PcUBI | GC | 15 | 79 |
| | targeted | | acid | | | | | |
| 113714 | plastidic | At2g36580 | linoleic | ARA_LEAF | p-PcUBI | GC | 14 | 19 |
| | | | acid | | | | | |
| 1623 | non- | At2g45420 | linoleic | ARA_LEAF | p-PcUBI | GC | 16 | 42 |
| | targeted | | acid | | | | | |
| 68657 | non- | At3g02990 | linoleic | ARA_LEAF | p-PcUBI | GC | 19 | 30 |
| | targeted | | acid | | | | | |
| 22921 | non- | At3g20910 | linoleic | ARA_LEAF | p-PcUBI | GC | 14 | 29 |
| | targeted | | acid | | | | | |
| 2935 | non- | At3g62950 | linoleic | ARA_LEAF | p-PcUBI | GC | 16 | 30 |
| | targeted | | acid | | | | | |
| 109717 | non- | At4g09960 | linoleic | ARA_LEAF | p-PcUBI | GC | 16 | 32 |
| | targeted | | acid | | | | | |
| 23482 | non- | At4g15660 | linoleic | ARA_LEAF | p-PcUBI | GC | 16 | 57 |
| | targeted | | acid | | | | | |
| 3279 | non- | At4g15670 | linoleic | ARA_LEAF | p-PcUBI | GC | 21 | 59 |
| | targeted | | acid | | | | | |
| 68849 | non- | At4g26080 | linoleic | ARA_LEAF | p-PcUBI | GC | 16 | 23 |
| | targeted | | acid | | | | | |
| 4102 | non- | At4g33040 | linoleic | ARA_LEAF | p-PcUBI | GC | 14 | 39 |
| | targeted | | acid | | | | | |
| 24438 | non- | At5g03720 | linoleic | ARA_LEAF | p-PcUBI | GC | 22 | 42 |
| | targeted | | acid | | | | | |
| 25498 | non- | At5g59220 | linoleic | ARA_LEAF | p-PcUBI | GC | 18 | 48 |
| | targeted | | acid | | | | | |
| 114083 | non- | Avin-DRAFT_1788 | linoleic | ARA_LEAF | p-PcUBI | GC | 15 | 27 |
| | targeted | | acid | | | | | |
| 114198 | non- | Avin-DRAFT_2010 | linoleic | ARA_LEAF | p-PcUBI | GC | 15 | 38 |
| | targeted | | acid | | | | | |
| 6040 | non- | Avin-DRAFT_2091 | linoleic | ARA_LEAF | p-PcUBI | GC | 18 | 29 |
| | targeted | | acid | | | | | |
| 29500 | non- | Avin-DRAFT_3253 | linoleic | ARA_LEAF | p-PcUBI | GC | 14 | 44 |
| | targeted | | acid | | | | | |
| 111155 | non- | Avin-DRAFT_3577 | linoleic | ARA_LEAF | p-PcUBI | GC | 15 | 43 |
| | targeted | | acid | | | | | |
| 114231 | non- | Avin-DRAFT_3629 | linoleic | ARA_LEAF | p-PcUBI | GC | 16 | 30 |
| | targeted | | acid | | | | | |
| 6510 | non- | Avin-DRAFT_5103 | linoleic | ARA_LEAF | p-PcUBI | GC | 20 | 36 |
| | targeted | | acid | | | | | |
| 7686 | non- | B0486 | linoleic | ARA_LEAF | p-Super | GC | 16 | 26 |
| | targeted | | acid | | | | | |
| 75807 | non- | B0801 | linoleic | ARA_LEAF | p-Super | GC | 15 | 30 |
| | targeted | | acid | | | | | |
| 114552 | plastidic | B1095 | linoleic | ARA_LEAF | p-Super | GC | 18 | 46 |
| | | | acid | | | | | |
| 114979 | non- | B1102 | linoleic | ARA_LEAF | p-Super | GC | 15 | 21 |
| | targeted | | acid | | | | | |
| 38226 | non- | B1330 | linoleic | ARA_LEAF | p-Super | GC | 15 | 35 |
| | targeted | | acid | | | | | |
| 38266 | plastidic | B1431 | linoleic | ARA_LEAF | p-Super | GC | 15 | 40 |
| | | | acid | | | | | |
| 115056 | non- | B1470 | linoleic | ARA_LEAF | p-Super | GC | 14 | 39 |
| | targeted | | acid | | | | | |
| 78154 | non- | B1589 | linoleic | ARA_LEAF | p-Super | GC | 14 | 33 |
| | targeted | | acid | | | | | |
| 38300 | non- | B1597 | linoleic | ARA_LEAF | p-Super | GC | 14 | 35 |
| | targeted | | acid | | | | | |
| 115064 | plastidic | B1611 | linoleic | ARA_LEAF | p-Super | GC | 14 | 33 |
| | | | acid | | | | | |
| 38345 | non- | B1627 | linoleic | ARA_LEAF | p-Super | GC | 18 | 25 |
| | targeted | | acid | | | | | |
| 78753 | non- | B1670 | linoleic | ARA_LEAF | p-Super | GC | 13 | 33 |
| | targeted | | acid | | | | | |
| 78953 | non- | B1837 | linoleic | ARA_LEAF | p-Super | GC | 13 | 37 |
| | targeted | | acid | | | | | |
| 8920 | non- | B2032 | linoleic | ARA_LEAF | p-Super | GC | 15 | 74 |
| | targeted | | acid | | | | | |
| 112193 | non- | B2043 | linoleic | ARA_LEAF | p-Super | GC | 14 | 27 |
| | targeted | | acid | | | | | |
| 115595 | non- | B2099 | linoleic | ARA_LEAF | p-Super | GC | 16 | 31 |
| | targeted | | acid | | | | | |
| 39002 | non- | B2107 | linoleic | ARA_LEAF | p-Super | GC | 15 | 47 |
| | targeted | | acid | | | | | |
| 39040 | non- | B2178 | linoleic | ARA_LEAF | p-Super | GC | 16 | 40 |
| | targeted | | acid | | | | | |
| 39237 | non- | B2399 | linoleic | ARA_LEAF | p-Super | GC | 24 | 35 |
| | targeted | | acid | | | | | |
| 115628 | non- | B2439 | linoleic | ARA_LEAF | p-Super | GC | 20 | 34 |
| | targeted | | acid | | | | | |
| 40329 | non- | B2474 | linoleic | ARA_LEAF | p-Super | GC | 19 | 39 |
| | targeted | | acid | | | | | |
| 40665 | non- | B2613 | linoleic | ARA_LEAF | p-Super | GC | 14 | 48 |
| | targeted | | acid | | | | | |
| 115655 | non- | B2682 | linoleic | ARA_LEAF | p-Super | GC | 20 | 23 |
| | targeted | | acid | | | | | |
| 115719 | non- | B2790 | linoleic | ARA_LEAF | p-Super | GC | 17 | 25 |
| | targeted | | acid | | | | | |
| 115791 | plastidic | B2836 | linoleic | ARA_LEAF | p-Super | GC | 19 | 43 |
| | | | acid | | | | | |
| 81980 | non- | B2849 | linoleic | ARA_LEAF | p-Super | GC | 15 | 40 |
| | targeted | | acid | | | | | |
| 115864 | non- | B2866 | linoleic | ARA_LEAF | p-Super | GC | 16 | 20 |
| | targeted | | acid | | | | | |
| 41006 | non- | B2909 | linoleic | ARA_LEAF | p-Super | GC | 20 | 28 |
| | targeted | | acid | | | | | |
| 116110 | non- | B3379 | linoleic | ARA_LEAF | p-Super | GC | 16 | 34 |
| | targeted | | acid | | | | | |
| 116172 | plastidic | B3451 | linoleic | ARA_LEAF | p-Super | GC | 14 | 32 |
| | | | acid | | | | | |
| 10726 | non- | B3823 | linoleic | ARA_LEAF | p-Super | GC | 14 | 42 |
| | targeted | | acid | | | | | |
| 100675 | plastidic | B3845 | linoleic | ARA_SEED_2 | p-USP | GC | 11 | 29 |
| | | | acid | | | | | |
| 116312 | non- | GM02LC38418 | linoleic | ARA_LEAF | p-PcUBI | GC | 14 | 23 |
| | targeted | | acid | | | | | |
| 85723 | mitochondrial | Sll1032 | linoleic | ARA_LEAF | p-PcUBI | GC | 16 | 22 |
| | | | acid | | | | | |
| 52660 | plastidic | Sll1056 | linoleic | ARA_LEAF | p-PcUBI | GC | 18 | 40 |
| | | | acid | | | | | |
| 53878 | mitochondrial | Sll1443 | linoleic | ARA_LEAF | p-PcUBI | GC | 15 | 33 |
| | | | acid | | | | | |
| 95047 | non- | Sll1498 | linoleic | ARA_LEAF | p-PcUBI | GC | 18 | 34 |
| | targeted | | acid | | | | | |
| 11471 | mitochondrial | Sll1917 | linoleic | ARA_LEAF | p-PcUBI | GC | 14 | 29 |
| | | | acid | | | | | |
| 55385 | mitochondrial | Sll1920 | linoleic | ARA_LEAF | p-PcUBI | GC | 14 | 22 |
| | | | acid | | | | | |
| 11990 | non- | Slr0338 | linoleic | ARA_LEAF | p-PcUBI | GC | 14 | 42 |
| | targeted | | acid | | | | | |
| 87655 | plastidic | Slr0966 | linoleic | ARA_LEAF | p-PcUBI | GC | 17 | 43 |
| | | | acid | | | | | |
| 116341 | mitochondrial | Slr1099 | linoleic | ARA_LEAF | p-PcUBI | GC | 14 | 44 |
| | | | acid | | | | | |
| 116460 | mitochondrial | Slr1250 | linoleic | ARA_LEAF | p-PcUBI | GC | 14 | 29 |
| | | | acid | | | | | |
| 58058 | non- | Slr1269 | linoleic | ARA_LEAF | p-PcUBI | GC | 18 | 24 |
| | targeted | | acid | | | | | |
| 58324 | non- | Slr1312 | linoleic | ARA_LEAF | p-PcUBI | GC | 14 | 45 |
| | targeted | | acid | | | | | |
| 116713 | non- | Slr1350 | linoleic | ARA_LEAF | p-PcUBI | GC | 14 | 29 |
| | targeted | | acid | | | | | |
| 12341 | mitochondrial | Slr2072 | linoleic | ARA_LEAF | p-PcUBI | GC | 13 | 35 |
| | | | acid | | | | | |
| 116848 | non- | TTC0490 | linoleic | ARA_LEAF | p-PcUBI | GC | 16 | 31 |
| | targeted | | acid | | | | | |
| 61553 | non- | TTC0917 | linoleic | ARA_LEAF | p-PcUBI | GC | 23 | 98 |
| | targeted | | acid | | | | | |
| 61723 | non- | TTC1193 | linoleic | ARA_LEAF | p-PcUBI | GC | 17 | 22 |
| | targeted | | acid | | | | | |
| 116948 | plastidic | Yal044c | linoleic | ARA_LEAF | p-Super | GC | 17 | 29 |
| | | | acid | | | | | |
| 14275 | plastidic | Ydl159w | linoleic | ARA_LEAF | p-Super | GC | 16 | 34 |
| | | | acid | | | | | |
| 117352 | plastidic | Ydr158w | linoleic | ARA_LEAF | p-PcUBI | GC | 14 | 38 |
| | | | acid | | | | | |
| 90165 | non- | Ydr507c | linoleic | ARA_LEAF | Big35S | GC | 14 | 34 |
| | targeted | | acid | | | | | |
| 117482 | non- | Ygr104c | linoleic | ARA_SEED_2 | p-PcUBI | GC | 15 | 19 |
| | targeted | | acid | | | | | |
| 64964 | plastidic | Yjl073w | linoleic | ARA_LEAF | p-Super | GC | 14 | 21 |
| | | | acid | | | | | |
| 66772 | non- | Ylr304c | linoleic | ARA_LEAF | Big35S | GC | 16 | 34 |
| | targeted | | acid | | | | | |
| 117495 | non- | Ynl064c | linoleic | ARA_LEAF | Big35S | GC | 15 | 25 |
| | targeted | | acid | | | | | |
| 118212 | plastidic | Ypl271w | linoleic | ARA_LEAF | p-Super | GC | 14 | 34 |
| | | | acid | | | | | |
| 16883 | plastidic | 59582753_SOYBEAN | linoleic | ARA_LEAF | p-PcUBI | GC | 14 | 37 |
| | | | acid | | | | | |

Column 1 shows the SEQ ID NO, Column 2 shows the expression type (targeted or non-targeted), Column 3 shows the “gene name” (locus), Column 4 shows the metabolite analyzed, Column 5 indicates the A. thaliana source tissue analyzed, Column 6 indicates the used promoter for expression, Column 7 indicates the analytical method. Columns 8 and 9 show the minimum and the maximum increase of the analyzed metabolite (in percent) in comparison to the wild type (=ratio_by_weight, given as percent increase).

When the analyses were repeated independently, all results proved to be significant.

### Example 12

### Engineering Arabidopsis Plants with an Increased Production of a Fine Chemical by (Over)Expressing a FCRP-Protein Encoded by a Gene from Saccharomyces Cerevisiae or Synechocystis or E. Coli, Azotobacter Vinelandii, Thermus Thermophilus, Physcomitrella Patens, Arabidopsis. Thaliana, Brassica Napus, Glycine Max, Zea Mays or Oryza Sativa using Tissue-Specific and/or Stress Inducible Promoters

Transgenic Arabidopsis plants are created as in example 11 to express the FCRP under the control of a tissue-specific and/or stress inducible promoter.

T2 generation plants are produced and are grown under standard conditions. The fine chemical production is determined after a total time of 29 to 30 days starting with the sowing. The transgenic Arabidopsis plant produces more fine chemical then non-transgenic control plants.

### Example 13

### Engineering Alfalfa Plants with an Increased Production of a Fine Chemical by (Over)Expressing a FCRP-Protein Encoded by a Gene From Saccharomyces Cerevisiae or Synechocystis or E. Coli, Azotobacter Vinelandii, Thermus Thermophilus, Physcomitrella Patens, Arabidopsis. Thaliana, Brassica Napus, Glycine Max, Zea Mays or Oryza Sativa

A regenerating clone of alfalfa (Medicago sativa) is transformed using the method of (McKersie et al., Plant Physiol 119, 839 (1999)). Regeneration and transformation of alfalfa is genotype dependent and therefore a regenerating plant is required. Methods to obtain regenerating plants have been described. For example, these can be selected from the cultivar Rangelander (Agriculture Canada) or any other commercial alfalfa variety as described by Brown D. C. W. and Atanassov A. (Plant Cell Tissue Organ Culture 4, 111 (1985)). Alternatively, the RA3 variety (University of Wisconsin) is selected for use in tissue culture (Walker et al., Am. J. Bot. 65, 654 (1978)).

Petiole explants are cocultivated with an overnight culture of Agrobacterium tumefaciens C58C1 pMP90 (McKersie et al., Plant Physiol 119, 839 (1999)) or LBA4404 containing a binary vector. Many different binary vector systems have been described for plant transformation (e.g. G. An, in “Agrobacterium Protocols, Methods in Molecular Biology”, Vol. 44, pp. 47-62, Gartland K. M. A. and Davey M. R., eds. Humana Press, Totowa, N.J.). Many are based on the vector pBI N19 described by Bevan (Nucleic Acid Research. 12, 8711 (1984)) that includes a plant gene expression cassette flanked by the left and right border sequences from the Ti plasmid of Agrobacterium tumefaciens. A plant gene expression cassette consists of at least two genes—a selection marker gene and a plant promoter regulating the transcription of the cDNA or genomic DNA of the trait gene. Various selection marker genes can be used including the Arabidopsis gene encoding a mutated acetohydroxy acid synthase (AHAS) enzyme (U.S. Pat. No. 5,7673,666 and U.S. Pat. No. 6,225,105). Similarly, various promoters can be used to regulate the trait gene that provides constitutive, developmental, tissue or environmental regulation of gene transcription. In this example, the 34S promoter (GenBank Accession numbers M59930 and X16673) is used to provide constitutive expression of the trait gene.

The explants are cocultivated for 3 days in the dark on SH induction medium containing 288 mg/L Pro, 53 mg/L thioproline, 4.35 g/L K₂SO₄, and 100 μm acetosyringinone. The explants are washed in half-strength Murashige-Skoog medium (Murashige and Skoog, 1962) and plated on the same SH induction medium without acetosyringinone but with a suitable selection agent and suitable antibiotic to inhibit Agrobacterium growth. After several weeks, somatic embryos are transferred to BOi2Y development medium containing no growth regulators, no antibiotics, and 50 g/L sucrose. Somatic embryos are subsequently germinated on half-strength Murashige-Skoog medium. Rooted seedlings are transplanted into pots and grown in a greenhouse.

T1 or T2 generation plants are produced, grown and processed and evaluated for their fine chemical produced in analogy as described above.

### Example 14

### Engineering Ryegrass Plants with an Increased Production of Fine Chemical by (Over)-Expressing a FCRP encoded by a Gene from Saccharomyces Cerevisiae or Synechocystis or E. Coli, Azotobacter Vinelandii, Thermus Thermophilus, Physcomitrella Patens, Arabidopsis Thaliana, Brassica Napus, Glycine Max, Zea Mays or Oryza Sativa

Seeds of several different ryegrass varieties may be used as explant sources for transformation, including the commercial variety Gunne available from Svalof Weibull seed company or the variety Affinity. Seeds are surface-sterilized sequentially with 1% Tween-20 for 1 minute, 100% bleach for 60 minutes, 3 rinses with 5 minutes each with deionized and distilled H₂O, and then germinated for 3-4 days on moist, sterile filter paper in the dark. Seedlings are further sterilized for 1 minute with 1% Tween-20, 5 minutes with 75% bleach, and rinsed 3 times with double distilled H₂O, 5 min each.

Surface-sterilized seeds are placed on the callus induction medium containing Murashige and Skoog basal salts and vitamins, 20 g/L sucrose, 150 mg/L asparagine, 500 mg/L casein hydrolysate, 3 g/L Phytagel, 10 mg/L BAP, and 5 mg/L dicamba. Plates are incubated in the dark at 25° C. for 4 weeks for seed germination and embryogenic callus induction.

After 4 weeks on the callus induction medium, the shoots and roots of the seedlings are trimmed away, the callus is transferred to fresh media, maintained in culture for another 4 weeks, and then transferred to MSO medium in light for 2 weeks. Several pieces of callus (11-17 weeks old) are either strained through a 10 mesh sieve and put onto callus induction medium, or cultured in 100 ml of liquid ryegrass callus induction media (same medium as for callus induction with agar) in a 250 ml flask. The flask is wrapped in foil and shaken at 175 rpm in the dark at 23° C. for 1 week. Sieving the liquid culture with a 40-mesh sieve collected the cells. The fraction collected on the sieve is plated and cultured on solid ryegrass callus induction medium for 1 week in the dark at 25° C. The callus is then transferred to and cultured on MS medium containing 1% sucrose for 2 weeks.

Transformation can be accomplished with either Agrobacterium of with particle bombardment methods. An expression vector is created containing a constitutive plant promoter and the cDNA of the gene in a pUC vector. The plasmid DNA is prepared from E. coli cells using with Qiagen kit according to manufacturer's instruction. Approximately 2 g of embryogenic callus is spread in the center of a sterile filter paper in a Petri dish. An aliquot of liquid MSO with 10 g/L sucrose is added to the filter paper. Gold particles (1.0 μm in size) are coated with plasmid DNA according to method of Sanford et al., 1993 and delivered to the embryogenic callus with the following parameters: 500 μg particles and 2 pg DNA per shot, 1300 psi and a target distance of 8.5 cm from stopping plate to plate of callus and 1 shot per plate of callus.

After the bombardment, calli are transferred back to the fresh callus development medium and maintained in the dark at room temperature for a 1-week period. The callus is then transferred to growth conditions in the light at 25° C. to initiate embryo differentiation with the appropriate selection agent, e.g. 250 nM Arsenal, 5 mg/L PPT or 50 mg/L kanamycin. Shoots resistant to the selection agent are appearing and once rooted are transferred to soil.

Samples of the primary transgenic plants (T0) are analyzed by PCR to confirm the presence of T-DNA. These results are confirmed by Southern hybridization in which DNA is electrophoresed on a 1% agarose gel and transferred to a positively charged nylon membrane (Roche Diagnostics). The PCR DIG Probe Synthesis Kit (Roche Diagnostics) is used to prepare a digoxigenin-labelled probe by PCR, and used as recommended by the manufacturer.

Transgenic T0 ryegrass plants are propagated vegetatively by excising tillers. The transplanted tillers are maintained in the greenhouse for 2 months until well established. The shoots are defoliated and allowed to grow for 2 weeks.

T1 or T2 generation plants are produced, grown and processed and evaluated for their fine chemical produced in analogy as described above to determine the fine chemical content.

### Example 15

### Engineering Soybean Plants with an Increased Production of Fine Chemical by (Over)-Expressing a FCRP Encoded by a Gene from Saccharomyces Cerevisiae or Synechocystis or E. Coli, Azotobacter Vinelandii, Thermus Thermophilus, Physcomitrella Patens, Arabidopsis Thaliana, Brassica Napus, Glycine Max, Zea Mays or Oryza Sativa

Soybean is transformed according to the following modification of the method described in the Texas A & M U.S. Pat. No. 5,164,310. Several commercial soybean varieties are amenable to transformation by this method. The cultivar Jack (available from the Illinois Seed Foundation) is commonly used for transformation. Seeds are sterilized by immersion in 70% (v/v) ethanol for 6 min and in 25% commercial bleach (NaOCI) supplemented with 0.1% (v/v) Tween for 20 min, followed by rinsing 4 times with sterile double distilled water. Seven-day seedlings are propagated by removing the radicle, hypocotyl and one cotyledon from each seedling. Then, the epicotyl with one cotyledon is transferred to fresh germination media in petri dishes and incubated at 25 ° C. under a 16-h photoperiod (approx. 100 μmol m⁻²s⁻¹) for three weeks. Axillary nodes (approx. 4 mm in length) were cut from 3-4 week-old plants. Axillary nodes are excised and incubated in Agrobacterium LBA4404 culture.

Many different binary vector systems have been described for plant transformation (e.g. An G., in Agrobacterium Protocols. Methods in Molecular Biology Vol. 44, pp. 47-62, Gartland K. M. A. and Davey M. R. eds. Humana Press, Totowa, N.J.). Many are based on the vector pBI N19 described by Bevan (Nucleic Acid Research. 12, 8711 (1984)) that includes a plant gene expression cassette flanked by the left and right border sequences from the Ti plasmid of Agrobacterium tumefaciens. A plant gene expression cassette consists of at least two genes—a selection marker gene and a plant promoter regulating the transcription of the cDNA or genomic DNA of the trait gene. Various selection marker genes can be used including the Arabidopsis gene encoding a mutated acetohydroxy acid synthase (AHAS) enzyme (U.S. Pat. No. 5,7673,666 and U.S. Pat. No. 6,225,105). Similarly, various promoters can be used to regulate the trait gene to provide constitutive, developmental, tissue or environmental regulation of gene transcription. In this example, the 34S promoter (GenBank Accession numbers M59930 and X16673) can be used to provide constitutive expression of the trait gene.

After the co-cultivation treatment, the explants are washed and transferred to selection media supplemented with 500 mg/L timentin. Shoots are excised and placed on a shoot elongation medium. Shoots longer than 1 cm are placed on rooting medium for two to four weeks prior to transplanting to soil.

The primary transgenic plants (TO) are analyzed by PCR to confirm the presence of T-DNA. These results are confirmed by Southern hybridization in which DNA is electrophoresed on a 1% agarose gel and transferred to a positively charged nylon membrane (Roche Diagnostics). The PCR DIG Probe Synthesis Kit (Roche Diagnostics) is used to prepare a digoxigenin-labelled probe by PCR, and used as recommended by the manufacturer.

T1 or T2 generation plants are produced, grown and processed and evaluated for their fine chemical produced in analogy as described above.

### Example 16

### Engineering Rapeseed/Canola Plants with an Increased Production of Fine Chemical by (Over)-Expressing a FCRP Encoded by a Gene from Saccharomyces Cerevisiae or Synechocystis or E. Coli, Azotobacter Vinelandii, Thermus Thermophilus, Physcomitrella Patens, Arabidopsis. Thaliana, Brassica Napus, Glycine Max, Zea Mays or Oryza Sativa

Cotyledonary petioles and hypocotyls of 5-6 day-old young seedlings are used as explants for tissue culture and transformed according to Babic et al. (Plant Cell Rep 17, 183 (1998)). The commercial cultivar Westar (Agriculture Canada) is the standard variety used for transformation, but other varieties can be used.

Agrobacterium tumefaciens LBA4404 containing a binary vector can be used for canola transformation. Many different binary vector systems have been described for plant transformation (e.g. An G., in Agrobacterium Protocols. Methods in Molecular Biology Vol. 44, p. 47-62, Gartland K. M. A.and Davey M. R. eds. Humana Press, Totowa, N.J.). Many are based on the vector pBI N19 described by Bevan (Nucleic Acid Research. 12, 8711(1984)) that includes a plant gene expression cassette flanked by the left and right border sequences from the Ti plasmid of Agrobacterium tumefaciens. A plant gene expression cassette consists of at least two genes—a selection marker gene and a plant promoter regulating the transcription of the cDNA or genomic DNA of the trait gene. Various selection marker genes can be used including the Arabidopsis gene encoding a mutated acetohydroxy acid synthase (AHAS) enzyme (U.S. Pat. No. 5,7673,666 and U.S. Pat. No. 6,225,105). Similarly, various promoters can be used to regulate the trait gene to provide constitutive, developmental, tissue or environmental regulation of gene transcription. In this example, the 34S promoter (GenBank Accession numbers M59930 and X16673) can be used to provide constitutive expression of the trait gene.

Canola seeds are surface-sterilized in 70% ethanol for 2 min., and then in 30% Clorox with a drop of Tween-20 for 10 min, followed by three rinses with sterilized distilled water. Seeds are then germinated in vitro 5 days on half strength MS medium without hormones, 1% sucrose, 0.7% Phytagar at 23° C., 16 h light. The cotyledon petiole explants with the cotyledon attached are excised from the in vitro seedlings, and inoculated with Agrobacterium by dipping the cut end of the petiole explant into the bacterial suspension. The explants are then cultured for 2 days on MSBAP-3 medium containing 3 mg/L BAP, 3% sucrose, 0.7% Phytagar at 23° C., 16 h light. After two days of co-cultivation with Agrobacterium, the petiole explants are transferred to MSBAP-3 medium containing 3 mg/L BAP, cefotaxime, carbenicillin, or timentin (300 mg/L) for 7 days, and then cultured on MSBAP-3 medium with cefotaxime, carbenicillin, or timentin and selection agent until shoot regeneration. When the shoots were 5-10 mm in length, they are cut and transferred to shoot elongation medium (MSBAP-0.5, containing 0.5 mg/L BAP). Shoots of about 2 cm in length are transferred to the rooting medium (MSO) for root induction.

Samples of the primary transgenic plants (T0) are analyzed by PCR to confirm the presence of T-DNA. These results are confirmed by Southern hybridization in which DNA is electrophoresed on a 1% agarose gel and transferred to a positively charged nylon membrane (Roche Diagnostics). The PCR DIG Probe Synthesis Kit (Roche Diagnostics) is used to prepare a digoxigenin-labelled probe by PCR, and used as recommended by the manufacturer.

T1 or T2 generation plants are produced, grown and processed and evaluated for their fine chemical produced in analogy as described above.

### Example 17

### Engineering corn plants with an Increased Production of Fine Chemical by (Over)-Expressing a FCRP Encoded by a Gene from Saccharomyces Cerevisiae or Synechocystis or E. Coli, Azotobacter Vinelandii, Thermus Thermophilus, Physcomitrella Patens, Arabidopsis Thaliana, Brassica Napus, Glycine Max, Zea Mays or Oryza Sativa

Transformation of maize (Zea Mays L.) is performed with a modification of the method described by Ishida et al. (Nature Biotech 14745 (1996)). Transformation is genotype-dependent in corn and only specific genotypes are amenable to transformation and regeneration. The inbred line A188 (University of Minnesota) or hybrids with A188 as a parent are good sources of donor material for transformation (Fromm et al., Biotech 8, 833 (1990)), but other genotypes can be used successfully as well. Ears are harvested from corn plants at approximately 11 days after pollination (DAP) when the length of immature embryos is about 1 to 1.2 mm. Immature embryos are co-cultivated with Agrobacterium tumefaciens that carry “super binary” vectors and transgenic plants are recovered through organogenesis. The super binary vector system of Japan Tobacco is described in WO patents WO 94/00977 and WO 95/06722. Vectors were constructed as described. Various selection marker genes can be used including the maize gene encoding a mutated acetohydroxy acid synthase (AHAS) enzyme (US patent 6,025,541). Similarly, various promoters can be used to regulate the trait gene to provide constitutive, developmental, tissue or environmental regulation of gene transcription. In this example, the 34S promoter (GenBank Accession numbers M59930 and X16673) was used to provide constitutive expression of the trait gene.

Excised embryos are grown on callus induction medium, then maize regeneration medium, containing imidazolinone as a selection agent. The Petri plates are incubated in the light at 25 ° C. for 2-3 weeks, or until shoots develop. The green shoots are transferred from each embryo to maize rooting medium and incubated at 25° C. for 2-3 weeks, until roots develop. The rooted shoots are transplanted to soil in the greenhouse. T1 seeds are produced from plants that exhibit tolerance to the imidazolinone herbicides and which are PCR positive for the transgenes.

The T1 transgenic plants are then evaluated for their fine chemical producted as described above. The T1 generation of single locus insertions of the T-DNA will segregate for the transgene in a 3:1 ratio. Those progeny containing one or two copies of the transgene are tolerant regarding the imidazolinone herbicide, and exhibit an increased fine chemical production then those progeny lacking the transgenes.

T1 or T2 generation plants are produced, grown, processed and evaluated for their fine chemial produced in analogy as described above. Homozygous T2 plants exhibited similar phenotypes. Hybrid plants (F1 progeny) of homozygous transgenic plants and non-transgenic plants also exhibited enhanced fine chemical production.

### Example 18

### Engineering Wheat Plants with an Increased Production of Fine Chemical by (Over)-Expressing a FCRP Encoded by a Gene from Saccharomyces Cerevisiae or Synechocystis or E. Coli, AzotoBacter Vinelandii, Thermus Thermophilus, Physcomitrella Patens, Arabidopsis Thaliana, Brassica Napus, Glycine Max, Zea Mays or Oryza Sativa

Transformation of wheat is performed with the method described by Ishida et al. (Nature Biotech. 14745 (1996)). The cultivar Bobwhite (available from CYMMIT, Mexico) is commonly used in transformation. Immature embryos are co-cultivated with Agrobacterium tumefaciens that carry “super binary” vectors, and transgenic plants are recovered through organogenesis. The super binary vector system of Japan Tobacco is described in WO patents WO 94/00977 and WO 95/06722. Vectors were constructed as described. Various selection marker genes can be used including the maize gene encoding a mutated acetohydroxy acid synthase (AHAS) enzyme (U.S. Pat. No. 6,025,541). Similarly, various promoters can be used to regulate the trait gene to provide constitutive, developmental, tissue or environmental regulation of gene transcription. In this example, the 34S promoter (GenBank Accession numbers M59930 and X16673) was used to provide constitutive expression of the trait gene.

After incubation with Agrobacterium, the embryos are grown on callus induction medium, then regeneration medium, containing imidazolinone as a selection agent. The Petri plates are incubated in the light at 25 ° C. for 2-3 weeks, or until shoots develop. The green shoots are transferred from each embryo to rooting medium and incubated at 25 ° C. for 2-3 weeks, until roots develop. The rooted shoots are transplanted to soil in the greenhouse. T1 seeds are produced from plants that exhibit tolerance to the imidazolinone herbicides and which are PCR positive for the transgenes.

The T1 transgenic plants are then evaluated for their enhanced production of fine chemical according to the methods described in the previous examples. The T1 generation of single locus insertions of the T-DNA will segregate for the transgene in a 3:1 ratio. Those progeny containing one or two copies of the transgene are tolerant regarding the imidazolinone herbicide, and exhibit an enhanced production of fine chemical then those progeny lacking the transgenes. Homozygous T2 plants exhibited similar phenotypes.

### Example 19

### Engineering Rice Plants with an Increased Production of Fine Chemical by (Over)-Expressing a FCRP Encoded by a Gene from Saccharomyces Cerevisiae or Synechocystis or E. Coli, Azotobacter Vinelandii, Thermus Thermophilus, Physcomitrella Patens, Arabidopsis Thaliana, Brassica Napus, Glycine Max, Zea Mays or Oryza Sativa

The Agrobacterium containing the expression vector of the invention is used to transform Oryza sativa plants. Mature dry seeds of the rice japonica cultivar Nipponbare are dehusked. Sterilization is carried out by incubating for one minute in 70% ethanol, followed by 30 minutes in 0.2% HgC12, followed by a 6 times 15 minutes can beh with sterile distilled water. The sterile seeds are then germinated on a medium containing 2,4-D (callus induction medium). After incubation in the dark for four weeks, embryogenic, scutellum-derived calli are excised and propagated on the same medium. After two weeks, the calli are multiplied or propagated by subculture on the same medium for another 2 weeks. Embryogenic callus pieces are sub-cultured on fresh medium 3 days before co-cultivation (to boost cell division activity).

Agrobacterium strain LBA4404 containing the expression vector of the invention is used for cocultivation. Agrobacterium is inoculated on AB medium with the appropriate antibiotics and cultured for 3 days at 28° C. The bacteria are then collected and suspended in liquid co-cultivation medium to a density (0D600) of about 1. The suspension is then transferred to a Petri dish and the calli immersed in the suspension for 15 minutes. The callus tissues are then blotted dry on a filter paper and transferred to solidified, co-cultivation medium and incubated for 3 days in the dark at 25° C. Co-cultivated calli are grown on 2,4-D-containing medium for 4 weeks in the dark at 28° C. in the presence of a selection agent. During this period, rapidly growing resistant callus islands developed. After transfer of this material to a regeneration medium and incubation in the light, the embryogenic potential is released and shoots developed in the next four to five weeks.

Shoots are excised from the calli and incubated for 2 to 3 weeks on an auxin-containing medium from which they are transferred to soil. Hardened shoots are grown under high humidity and short days in a greenhouse.

Approximately 35 independent TO rice transformants are generated for one construct. The primary transformants are transferred from a tissue culture chamber to a greenhouse. After a quantitative PCR analysis to verify copy number of the T-DNA insert, only single copy transgenic plants that exhibit tolerance to the selection agent are kept for harvest of T1 seed. Seeds are then harvested three to five months after transplanting. The method yielded single locus transformants at a rate of over 50% (Aldemita and Hodges1996, Chan et al. 1993, Hiei et al. 1994).

T1 or T2 generation plants are produced, grown, processed and evaluated for their fine chemical produced in analogy as described above

### Example 20

### Identification of Identical and Heterologous Genes

Gene sequences can be used to identify identical or heterologous genes from cDNA or genomic libraries. Identical genes (e. g. full-length cDNA clones) can be isolated via nucleic acid hybridization using for example cDNA libraries. Depending on the abundance of the gene of interest, 100,000 up to 1,000,000 recombinant bacteriophages are plated and transferred to nylon membranes. After denaturation with alkali, DNA is immobilized on the membrane by e. g. UV cross linking. Hybridization is carried out at high stringency conditions. In aqueous solution, hybridization and washing is performed at an ionic strength of 1 M NaCl and a temperature of 68° C. Hybridization probes are generated by e.g. radioactive (32P) nick transcription labeling (High Prime, Roche, Mannheim, Germany). Signals are detected by autoradiography.

Partially identical or heterologous genes that are related but not identical can be identified in a manner analogous to the above-described procedure using low stringency hybridization and washing conditions. For aqueous hybridization, the ionic strength is normally kept at 1 M NaCl while the temperature is progressively lowered from 68 to 42° C.

Isolation of gene sequences with homology (or sequence identity/similarity) only in a distinct domain of (for example 10-20 amino acids) can be carried out by using synthetic radio labeled oligonucleotide probes. Radiolabeled oligonucleotides are prepared by phosphorylation of the 5-prime end of two complementary oligonucleotides with T4 polynucleotide kinase. The complementary oligonucleotides are annealed and ligated to form concatemers. The double stranded concatemers are than radiolabeled by, for example, nick transcription. Hybridization is normally performed at low stringency conditions using high oligonucleotide concentrations.

### Oligonucleotide Hybridization Solution:

6×SSC
0.01 M sodium phosphate
1 mM EDTA (pH 8)
0.5% SDS
100 pg/ml denatured salmon sperm DNA
0.1% nonfat dried milk

During hybridization, temperature is lowered stepwise to 5-10° C. below the estimated oligonucleotide Tm or down to room temperature followed by washing steps and autoradiography. Washing is performed with low stringency such as 3 washing steps using 4×SSC. Further details are described by Sambrook J. et al., 1989, “Molecular Cloning: A Laboratory Manual,” Cold Spring Harbor Laboratory Press or Ausubel F.M. et al., 1994, “Current Protocols in Molecular Biology,” John Wiley & Sons.

### Example 21

### Identification of Identical Genes by Screening Expression Libraries with Antibodies

c-DNA clones can be used to produce recombinant polypeptide for example in E. coli (e.g. Qiagen QlAexpress pQE system). Recombinant polypeptides are then normally affinity purified via Ni-NTA affinity chromatography (Qiagen). Recombinant polypeptides are then used to produce specific antibodies for example by using standard techniques for rabbit immunization. Antibodies are affinity purified using a Ni-NTA column saturated with the recombinant antigen as described by Gu et al., BioTechniques 17, 257 (1994). The antibody can than be used to screen expression cDNA libraries to identify identical or heterologous genes via an immunological screening (Sambrook J., et al., “Molecular Cloning: A Laboratory Manual,” Cold Spring Harbor Laboratory Press, 1989, or Ausubel F. M. et al., “Current Protocols in Molecular Biology”, John Wiley & Sons, 1994,).

### FIGURES

FIG. 1. Vector VC-MME220-1 qcz (SEQ ID NO: 41) used for cloning gene of interest for non-targeted expression.

FIG. 2. Vector VC-MME221-1qcz (SEQ ID NO: 46) used for cloning gene of interest for non-targeted expression.

FIG. 3. Vector VC-MME354-1 QCZ (SEQ ID NO: 32) used for cloning gene of interest for plastidic targeted expression.

FIG. 4. Vector VC-MME432-1qcz (SEQ ID NO: 42) used for cloning gene of interest for plastidic targeted expression.

FIG. 5. Vector VC-MME489-1 QCZ (SEQ ID NO: 56) used for cloning gene of interest for non-targeted expression and cloning of a targeting sequence.

FIG. 6. Vector pMTX0270p (SEQ ID NO: 30) used for cloning of a targeting sequence.

FIG. 7. Vector pMTX155 (SEQ ID NO: 31) used for used for cloning gene of interest for non-targeted expression.

FIG. 8. Vector VC-MME356-1 QCZ (SEQ ID NO: 34) used for mitochondric targeted expression.

FIG. 9. Vector VC-MME301-1 QCZ (SEQ ID NO: 36) used for non-targeted expression in preferentially seeds.

FIG. 10. Vector pMTX461korrp (SEQ ID NO: 37) used for plastidic targeted expression in preferentially seeds.

FIG. 11. Vector VC-MME462-1 QCZ (SEQ ID NO: 39) used for mitochondric targeted expression in preferentially seeds.

FIG. 12. Vector VC-MME431-1qcz (SEQ ID NO: 44) used for mitochondric targeted expression.

FIG. 13. Vector pMTX447korr (SEQ ID NO: 47) used for plastidic targeted expression.

FIG. 14. Vector VC-MME445-1 qcz (SEQ ID NO: 49) used for mitochondric targeted expression.

FIG. 15. Vector VC-MME289-1 qcz (SEQ ID NO: 51) used for non targeted expression in preferentially seeds. FIG. 16. Vector VC-MME464-1 qcz (SEQ ID NO: 52) used for plastidic targeted expression in preferentially seeds.

FIG. 17. Vector VC-MME465-1 qcz (SEQ ID NO: 54) used for mitochondric targeted expression in preferentially seeds.

In a further embodiment, the present invention relates in paragraphs [0000.1.9.9.] to [0514.1.8.9.] to a further process for the production of the fine chemical linolenic acid as defined below and corresponding embodiments as described herein as follows.

In a further embodiment, the present invention relates to a further process for the production of the fine chemical linolenic acid as defined below and corrresponding embodiments as described herein as follows.

[0001.1.8.9] to [008.1.8.9] for the disclosure of these paragraphs see [0001.1.8.8] to [008.1.8.8] above.

As described above, the essential fatty acids are necessary for humans and many mammals, for example for livestock.Essential fatty acids, such as alpha-linolenic acid, are extremely important for healing as well as maintaining good health. Compounds made from alpha-linolenic acid have been shown to decrease blood clotting and to decrease inflammatory proceses in the boody.

[0010.1.8.9] to [0011.1.8.9] for the disclosure of these paragraphs see [0010.1.8.8] to [0011.1.8.8] above.

It is an object of the present invention to develop an inexpensive process for the synthesis of linolenic acid. linolenic acid is one of the fatty acids , which are most frequently limiting.

for the disclosure of this paragraph see [0013.1.8.8] above.

Accordingly, in a first embodiment, the invention relates to a process for the production of at least one fine chemical selected from the group consisting of: linolenic acid, or, in other words, of the “fine chemical” or “fine chemical of the invention”.

The terms “fine chemical of the invention”, “fine chemical” or “the fine chemical” are used herein equally and relate in context of the paragraphs or sections [0014.1.9.9] to [0514.1.8.9] essentially to the metabolite or the metabolites indicated in column 7, application no. 9 of Tables I, II and IV in the respective line.

Further, the term “in context of any of the paragraphs [0014.1.9.9] to [0514.1.8.9]” as used herein means that for any of said paragraphs [0014.1.9.9] to [0514.1.8.9] the term “the fine chemical” is understood to follow the definition of paragraphs or sections [0014.1.9.9] and [0015.1.9.9], independently whether it refers to any other paragraph or not and whether the reference recites the term “fine chemical” in an other context.

Thus, in cases where one or more paragraphs or sections are incorporated by reference into any of the present paragraphs [0014.1.9.9] to [0514.1.8.9], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to the definition of paragraph [0014.1.9.9] and [0015.1.9.9].

Accordingly, in one embodiment, the term “fine chemical of the invention”, “fine chemical” or “the fine chemical” as used herein (alone or in combination, like in FCRP) means “linolenic acid in context of the nucleic acid or polypeptide sequences listed in the respective same line of any one of Tables I to IV of application no. 9 and indicating in column 7 the metabolite “linolenic acid”.

In one embodiment, the term linolenic acid or the term “fine chemical” mean in context of the paragraphs or sections [0014.1.9.9] to [0514.1.8.9] at least one chemical compound with an activity of the above mentioned linolenic acid, respectively.

Accordingly, in one embodiment, the terms “fine chemical of the invention”, “fine chemical” or “the fine chemical” mean in context of any of the paragraphs [0014.1.9.9] to

linolenic acid, its tryglycerides, lipids, oils and/or fats in free form or bound form. In a preferred embodiment, the term “the fine chemical” means linolenic acid in free form or e.g. in form of its tryglycerides, lipids, oils and/or fats. In a preferred embodiment “linolenic acid” means linolenic acid in free form.

In one embodiment, the term “the fine chemical” or “the respective fine chemical” mean in context of any of the paragraphs [0014.1.9.9] to [0514.1.8.9] linolenic acid and/or tryglycerides, lipids, oils and/or fats of linolenic acid, and its salts, ester, thioester or linolenic acid in free form or bound to other compounds such as triglycerides, glycolipids, phospholipids etc. In a preferred embodiment, the term “the fine chemical” means linolenic acid, in free form or its salts or bound to triglycerides. Triglycerides, lipids, oils, fats or lipid mixture thereof shall mean any triglyceride, lipid, oil and/or fat containing any bound or free linolenic acid for example sphingolipids, phosphoglycerides, lipids, glycolipids such as glycosphingolipids, phospholipids such as phosphatidylethanolamine, phosphatidylcholine, phosphatidylserine, phosphatidylglycerol, phosphatidylinositol or diphosphatidylglycerol, or as monoacylglyceride, diacylglyceride or triacylglyceride or other fatty acid esters such as acetyl-Coenzym A thioester, which contain further saturated or unsaturated fatty acids in the fatty acid molecule.

In one embodiment, the term “the fine chemical” and the term “the respective fine chemical” mean at least one chemical compound with an activity of the abovementioned fine chemical. Further, the term “in context of any of the paragraphs [0014.1.9.9] to [0514.1.8.9]” as used herein means that for any of said paragraphs [0014.1.9.9] to [0514.1.8.9] the term “the fine chemical” is understood to follow the definition of section [0014.1.9.9] or section [0015.1.9.9], independently whether it refers to any other paragraph or not and whether the reference recites the term “fine chemical” in an other context.

Thus, in cases where one or more paragraphs or sections are incorporated by reference into any of the present paragraphs [0014.1.9.9] to [0514.1.8.9], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to this definition of this paragraph [0015.1.9.9].

Further, the term “fine chemicals” as used herein relates to compositions comprising said fine chemical(s), i.e. comprising linolenic acid, respectively.

Accordingly, the present invention relates to a process for the production of linolenic acid, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of At2g45420-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of secindependent protein translocase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of acyl-CoA synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of ThiF family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of CTP synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of recombinase A in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of isopropylmalate isomerase large subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of HesB/YadR/YfhF family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of b0195-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of asparaginase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of multidrug resistance protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of 3-oxoacyl-[acyl-carrier-protein] reductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of 3-oxoacyl-[acyl-carrier-protein] synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of b1234-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of b1248-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of b1280-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of b1330-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of b1470-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of acid shock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of fumarate hydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of electron transport complex protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of b1670-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of b1672-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of b1837-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of b2032-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of b2107-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of b2399-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of ethanolamine utilization protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of b2474-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of b2613-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of b2682-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of flavodoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of AAS bifunctional protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of phosphotriesterase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of b3442-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of b3534-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of threonine efflux protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of b4023-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of branched chain amino acid ABC transporter ATP-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of phosphofructokinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of carbon dioxide concentrating mechanism protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of sll1032-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of CTP synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of carbamoyl-phosphate synthase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of acyl transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of coproporphyrinogen III oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of cation-transporting ATPase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of bifunctional purine biosynthesis protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of permease protein of ABC transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of aromatic acid decarboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of phosphate import ATP-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of arginine decarboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of phosphoadenosine phosphosulfate reductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of threonine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of metal-dependent hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of TTC1386-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of acid phosphatase precursor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of glycerol-3-phosphate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of cell division control protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of branched-chain amino acid permease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of F-box protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of ydr339c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of ydr507c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of dihydroxyacetone kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of DnaJ-like chaperone in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of ymr013c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of molecular chaperone in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of ATPase epsilon subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating one or more activities selected from the group consisting of glucose-6-phosphate 1-dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

An embodiment of the present invention relates to a process for the production of linolenic acid, which comprises
(a1) increasing or generating the activity of a polypeptide comprising a polypeptide as depicted in a respective line in column 5 or 8 of Table II, application no. 9, or a homolog or a fragment thereof, a consensus sequence or at least one polypeptide motif as depicted in the respective line in column 8 of Table IV, application no. 9; or
(a2) increasing or generating the activity of an expression product of one or more nucleic acid molecule(s) comprising a polynucleotide as depicted in the respective line in column 5 or 8 of Table I, application no. 9, preferably the coding region thereof, or a homolog or a fragment thereof; or
(a3) increasing or generating the activity of a functional equivalent of (a1) or (a2); in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of linolenic acid or a composition comprising linolenic acid in said non-human organism or in the culture medium surrounding said non-human organism.

Accordingly, the increase or generation of one or more said activities is for example conferred by one or more expression products of said nucleic acid molecule, e.g. proteins. Accordingly, in the present invention described above, the increase or generation of one or more said activities is for example conferred by one or more protein(s) each comprising a polypeptide selected from the group as depicted in Table II, application no. 9, column 5 and 8, or a homolog or a fragment thereof.

The process of the invention comprises in one embodiment the following steps:
(i) increasing or generating of the expression of; and/or
(ii) increasing or generating the expression of an expression product of; and/or
(iii) increasing or generating one or more activities of an expression product encoded by;
at least one nucleic acid molecule (in the following “Fine Chemical Related Protein (FCRP)”encoding gene or “FCRP”-gene) comprising a nucleic acid molecule selected from the group consisting of:
(a) a nucleic acid molecule encoding the polypeptide shown in column 5 or 8 of Table II, preferably Table II B, application no. 9, or a homolog or a fragment thereof;
(b) a nucleic acid molecule shown in column 5 or 8 of Table I, preferably Table I B, application no. 9, or a homolog or a fragment thereof (preferably the coding region thereof);
(c) a nucleic acid molecule, which, as a result of the degeneracy of the genetic code, can be derived from a polypeptide sequence depicted in column 5 or 8 of Table II, preferably Table II B, application no. 9;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 9, or the coding region thereof;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d),
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 9;
(i) a nucleic acid molecule encoding a polypeptide having the activity represented by a respective protein as depicted in column 5 of Table II, application no. 9;
(j) a nucleic acid molecule which comprises a polynucleotide, which is obtained by amplifying a cDNA library or a genomic library using the primers as depicted in column 8 of Table III, application no. 9; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

The term “increasing or generating of the expression of” may also be interchanged by the term “overexpressing”.

In a preferred embodiment thereof said nucleic acid molecule encodes a polypeptide which has the activity of the polypeptide represented by a protein comprising a polypeptide as depicted in the corresponding hit in column 5 of Table II, application no. 9.

In another preferred embodiment thereof said nucleic acid molecule confers a generation or an increase of the respective fine chemical in a non-human organism or a part thereof as compared to a corresponding wild-type non-human organism or part thereof.

In another preferred embodiment thereof said nucleic acid molecule encodes a polypeptide which has the activity of the polypeptide represented by a protein as depicted in the corresponding hit in column 5 of Table II, application no. 9, and confers a generation or an increase of the respective fine chemical in a non-human organism or a part thereof as compared to a corresponding wild-type non-human organism or part thereof.

Accordingly, the genes of the present invention or used in accordance with the present invention, which respectively encode a protein having an activity of 3-oxoacyl-[acyl-carrier-protein] reductase, 3-oxoacyl-[acyl-carrier-protein] synthase, AAS bifunctional protein, acid phosphatase precursor, acid shock protein, acyl transferase, acyl-CoA synthase, arginine decarboxylase, aromatic acid decarboxylase, asparaginase, At2g45420-protein, ATPase epsilon subunit, b0195-protein, b1234-protein, b1248-protein, b1280-protein, b1330-protein, b1470-protein, b1670-protein, b1672-protein, b1837-protein, b2032-protein, b2107-protein, b2399-protein, b2474-protein, b2613-protein, b2682-protein, b3442-protein, b3534-protein, b4023-protein, bifunctional purine biosynthesis protein, branched chain amino acid ABC transporter ATP-binding protein, branched-chain amino acid permease, carbamoyl-phosphate synthase subunit, carbon dioxide concentrating mechanism protein, cation-transporting ATPase, cell division control protein, coproporphyrinogen III oxidase, CTP synthase, CTP synthetase, dihydroxyacetone kinase, DnaJ-like chaperone, electron transport complex protein, ethanolamine utilization protein, F-box protein, flavodoxin, fumarate hydratase, glucose-6-phosphate 1-dehydrogenase, glutaredoxin, glycerol-3-phosphate dehydrogenase, heat shock transcription factor, HesB/YadR/YfhF family protein, isopropylmalate isomerase large subunit, metal-dependent hydrolase, molecular chaperone, monothiol glutaredoxin, multidrug resistance protein, oxidoreductase, permease protein of ABC transporter, phosphate import ATP-binding protein, phosphoadenosine phosphosulfate reductase , phosphofructokinase, phosphotriesterase, protein phosphatase, pyruvate kinase, recombinase A, sec-independent protein translocase, sll1032-protein, ThiF family protein, threonine dehydratase, threonine efflux protein, transcription factor, TTC1386-protein, ydr339c-protein, ydr507c-protein, or ymr013c-protein, which respectively encode a protein comprising a polypeptide encoded by a nucleic acid sequence as shown in Table I, application no. 9, column 5 or 8, (preferably the coding region thereof), or a homolog or a fragment thereof, which respectively encode a protein comprising a polypeptide as depicted in Table II, application no. 9, column 5 or 8, or a homolg or a fragment thereof, and/or which respectively can be amplified with the primer set shown in Table III, application no. 9, column 8, are also referred to as “FCRP genes”.

Proteins or polypeptides encoded by “FCRP-genes” are referred to as “Fine Chemical Related Proteins” or “FCRP”. For the purposes of the description of the present invention, the respective protein having an activity of 3-oxoacyl-[acyl-carrier-protein] reductase, 3-oxoacyl-[acyl-carrier-protein] synthase, AAS bifunctional protein, acid phosphatase precursor, acid shock protein, acyl transferase, acyl-CoA synthase, arginine decarboxylase, aromatic acid decarboxylase, asparaginase, At2g45420-protein, ATPase epsilon subunit, b0195-protein, b1234-protein, b1248-protein, b1280-protein, b1330-protein, b1470-protein, b1670-protein, b1672-protein, b1837-protein, b2032-protein, b2107-protein, b2399-protein, b2474-protein, b2613-protein, b2682-protein, b3442-protein, b3534-protein, b4023-protein, bifunctional purine biosynthesis protein, branched chain amino acid ABC transporter ATP-binding protein, branched-chain amino acid permease, carbamoyl-phosphate synthase subunit, carbon dioxide concentrating mechanism protein, cation-transporting ATPase, cell division control protein, coproporphyrinogen III oxidase, CTP synthase, CTP synthetase, dihydroxyacetone kinase, DnaJ-like chaperone, electron transport complex protein, ethanolamine utilization protein, F-box protein, flavodoxin, fumarate hydratase, glucose-6-phosphate 1-dehydrogenase, glutaredoxin, glycerol-3-phosphate dehydrogenase, heat shock transcription factor, HesB/YadR/YfhF family protein, isopropylmalate isomerase large subunit, metal-dependent hydrolase, molecular chaperone, monothiol glutaredoxin, multidrug resistance protein, oxidoreductase, permease protein of ABC transporter, phosphate import ATP-binding protein, phosphoadenosine phosphosulfate reductase , phosphofructokinase, phosphotriesterase, protein phosphatase, pyruvate kinase, recombinase A, sec-independent protein translocase, sll1032-protein, ThiF family protein, threonine dehydratase, threonine efflux protein, transcription factor, TTC1386-protein, ydr339c-protein, ydr507c-protein, or ymr013c-protein, the respective protein comprising a polypeptide encoded by one or more respective nucleic acid sequences as shown in Table I, application no. 9, column 5 or 8, (preferably the coding region thereof), or a homolog or fragment thereof, the respective protein comprising a respective polypeptide as depicted in Table II, application no. 9, column 5 or 8, or a homolog or fragment thereof, the respective protein comprising a sequence corresponding to the consensus sequence as shown in Table IV, application no. 9, column 8, and/or the respective protein comprising at least one polypeptide motif as shown in Table IV, application no. 9, column 8 is also referred to as Fine Chemical Related Protein” or “FCRP”.

Thus, in one embodiment, the present invention provides a process of the production of linolenic acid, by increasing or generating one or more activities, especially selected from the group consisting of 3-oxoacyl-[acyl-carrier-protein] reductase, 3-oxoacyl-[acyl-carrier-protein] synthase, AAS bifunctional protein, acid phosphatase precursor, acid shock protein, acyl transferase, acyl-CoA synthase, arginine decarboxylase, aromatic acid decarboxylase, asparaginase, At2g45420-protein, ATPase epsilon subunit, b0195-protein, b1234-protein, b1248-protein, b1280-protein, b1330-protein, b1470-protein, b1670-protein, b1672-protein, b1837-protein, b2032-protein, b2107-protein, b2399-protein, b2474-protein, b2613-protein, b2682-protein, b3442-protein, b3534-protein, b4023-protein, bifunctional purine biosynthesis protein, branched chain amino acid ABC transporter ATP-binding protein, branched-chain amino acid permease, carbamoyl-phosphate synthase subunit, carbon dioxide concentrating mechanism protein, cation-transporting ATPase, cell division control protein, coproporphyrinogen III oxidase, CTP synthase, CTP synthetase, dihydroxyacetone kinase, DnaJ-like chaperone, electron transport complex protein, ethanolamine utilization protein, F-box protein, flavodoxin, fumarate hydratase, glucose-6-phosphate 1-dehydrogenase, glutaredoxin, glycerol-3-phosphate dehydrogenase, heat shock transcription factor, HesB/YadR/YfhF family protein, isopropylmalate isomerase large subunit, metal-dependent hydrolase, molecular chaperone, monothiol glutaredoxin, multidrug resistance protein, oxidoreductase, permease protein of ABC transporter, phosphate import ATP-binding protein, phosphoadenosine phosphosulfate reductase, phosphofructokinase, phosphotriesterase, protein phosphatase, pyruvate kinase, recombinase A, sec-independent protein translocase, sll1032-protein, ThiF family protein, threonine dehydratase, threonine efflux protein, transcription factor, TTC1386-protein, ydr339c-protein, ydr507c-protein, and ymr013c-protein, which is conferred by one or more FCRPs or the gene product of one or more FCRP-genes, for example by the gene product of a nucleic acid sequences comprising a polynucleotide selected from the group as shown in Table I, application no. 9, column 5 or 8, (preferably by the coding region thereof), or a homolog or a fragment thereof, e.g. or by one or more proteins each comprising a polypeptide encoded by one or more nucleic acid sequences selected from the group as shown in Table I, application no. 9, column 5 or 8, (preferably by the coding region thereof), or a homolog or a fragment thereof, or by one or more protein(s) each comprising a polypeptide selected from the group as depicted in Table II, application no. 9, column 5 and 8, or a homolog thereof, or a protein comprising a sequence corresponding to the consensus sequence or comprising at least one polypeptide motif as shown in Table IV, application no. 9, column 8.

for the disclosure of this paragraph see [0025.1.8.8] above.

In an embodiment, the process comprises increasing or generating the activity of one or more polypeptides having said activity, e.g.

by generating or increasing the amount and/or specific activity in the cell or a compartment of a cell of one of more FCRP, especially selected from the group consisting of 3-oxoacyl-[acyl-carrier-protein] reductase, 3-oxoacyl-[acyl-carrier-protein] synthase, AAS bifunctional protein, acid phosphatase precursor, acid shock protein, acyl transferase, acyl-CoA synthase, arginine decarboxylase, aromatic acid decarboxylase, asparaginase, At2g45420-protein, ATPase epsilon subunit, b0195-protein, b1234-protein, b1248-protein, b1280-protein, b1330-protein, b1470-protein, b1670-protein, b1672-protein, b1837-protein, b2032-protein, b2107-protein, b2399-protein, b2474-protein, b2613-protein, b2682-protein, b3442-protein, b3534-protein, b4023-protein, bifunctional purine biosynthesis protein, branched chain amino acid ABC transporter ATP-binding protein, branched-chain amino acid permease, carbamoyl-phosphate synthase subunit, carbon dioxide concentrating mechanism protein, cation-transporting ATPase, cell division control protein, coproporphyrinogen III oxidase, CTP synthase, CTP synthetase, dihydroxyacetone kinase, DnaJ-like chaperone, electron transport complex protein, ethanolamine utilization protein, F-box protein, flavodoxin, fumarate hydratase, glucose-6-phosphate 1-dehydrogenase, glutaredoxin, glycerol-3-phosphate dehydrogenase, heat shock transcription factor, HesB/YadR/YfhF family protein, isopropylmalate isomerase large subunit, metal-dependent hydrolase, molecular chaperone, monothiol glutaredoxin, multidrug resistance protein, oxidoreductase, permease protein of ABC transporter, phosphate import ATP-binding protein, phosphoadenosine phosphosulfate reductase , phosphofructokinase, phosphotriesterase, protein phosphatase, pyruvate kinase, recombinase A, sec-independent protein translocase, sll1032-protein, ThiF family protein, threonine dehydratase, threonine efflux protein, transcription factor, TTC1386-protein, ydr339c-protein, ydr507c-protein, and ymr013c-protein, for example of the respective polypeptide as depicted in Table II, application no. 9, column 5 and 8, or a homolog or a fragment thereof, or the respective polypeptide comprising a sequence corresponding to the consensus sequences as shown in Table IV, application no. 9, column 8, or the respective polypeptide comprising at least one polypeptide motif as depicted in Table IV, application no. 9, column 8.

A further embodiment of the present invention relates to a process for the production of linolenic acid, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a At2g45420-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a sec-independent protein translocase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a acyl-CoA synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a ThiF family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a CTP synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a recombinase A non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a isopropylmalate isomerase large subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a HesB/YadR/YfhF family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a b0195-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a asparaginase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a multidrug resistance protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a 3-oxoacyl-[acyl-carrier-protein] reductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a 3-oxoacyl-[acyl-carrier-protein] synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a b1234-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a b1248-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a b1280-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a b1330-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a b1470-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a acid shock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a fumarate hydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a electron transport complex protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a b1670-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a b1672-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a b1837-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a b2032-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a b2107-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a b2399-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a ethanolamine utilization protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a b2474-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a b2613-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a b2682-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a flavodoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a AAS bifunctional protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a phosphotriesterase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a b3442-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a b3534-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a threonine efflux protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a b4023-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a branched chain amino acid ABC transporter

ATP-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a phosphofructokinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a carbon dioxide concentrating mechanism protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a sill 032-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a CTP synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a carbamoyl-phosphate synthase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a acyl transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a coproporphyrinogen III oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a cation-transporting ATPase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a bifunctional purine biosynthesis protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a permease protein of ABC transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a aromatic acid decarboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a phosphate import ATP-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a arginine decarboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a phosphoadenosine phosphosulfate reductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a threonine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a metal-dependent hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a TTC1386-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a acid phosphatase precursor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a glycerol-3-phosphate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a cell division control protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a branched-chain amino acid permease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a F-box protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a ydr339c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a ydr507c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a dihydroxyacetone kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a DnaJ-like chaperone non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a ymr013c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a molecular chaperone non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a ATPase epsilon subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a glucose-6-phosphate 1-dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating the activity of a polypeptide comprising a polypeptide as depicted in the respective line in column 5 or 8 of Table II, application no. 9, or a homolog or a fragment thereof, a consensus sequence or at least one polypeptide motif as depicted in the respective line in column 8 of Table IV, application no. 9; or
increasing or generating the activity of an expression product of one or more nucleic acid molecule(s) comprising a polynucleotide as depicted in the respective line in column 5 or 8 of Table I, application no. 9, preferably the coding region thereof, or a homolog or a fragment thereof;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of linolenic acid, or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of At2g45420-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of secindependent protein translocase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of acyl-CoA synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of ThiF family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of CTP synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of recombinase A in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of isopropylmalate isomerase large subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of HesB/YadR/YfhF family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of b0195-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of asparaginase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of multidrug resistance protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of 3-oxoacyl-[acyl-carrier-protein] reductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of 3-oxoacyl-[acyl-carrier-protein] synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of b1234-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of b1248-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of b1280-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of b1330-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of b1470-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of acid shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of fumarate hydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of electron transport complex protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of b1670-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of b1672-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of b1837-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of b2032-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of b2107-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of b2399-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of ethanolamine utilization protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of b2474-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of b2613-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of b2682-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of flavodoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of AAS bifunctional protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of phosphotriesterase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of b3442-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of b3534-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of threonine efflux protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of b4023-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of branched chain amino acid ABC transporter ATP-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of phosphofructokinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of carbon dioxide concentrating mechanism protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of sll1032-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of CTP synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of carbamoyl-phosphate synthase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of acyl transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of coproporphyrinogen III oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of cation-transporting ATPase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of bifunctional purine biosynthesis protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of permease protein of ABC transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of aromatic acid decarboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of phosphate import ATP-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of arginine decarboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of phosphoadenosine phosphosulfate reductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of threonine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of metal-dependent hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of TTC1386-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of acid phosphatase precursor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of glycerol-3-phosphate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of cell division control protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of branched-chain amino acid permease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of F-box protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of ydr339c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of ydr507c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of dihydroxyacetone kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of DnaJ-like chaperone in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of ymr013c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of molecular chaperone in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of ATPase epsilon subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a) increasing or generating one or more activities selected from the group consisting of glucose-6-phosphate 1-dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

A further embodiment of the present invention relates to a process for the production of linolenic acid, which comprises
(a1) increasing or generating the activity of a polypeptide comprising a polypeptide as depicted in the respective line in column 5 or 8 of Table II, application no. 9, or a homolog or fragment thereof, a consensus sequence or at least one polypeptide motif as depicted in column 8 of Table IV, application no. 9; or
increasing or generating the activity of an expression product of one or more nucleic acid molecule(s) comprising a polynucleotide as depicted in the respective line in column 5 or 8 of Table I, application no. 9, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 9, or a homolog or a fragment thereof, a consensus sequence or at least one polypeptide motif as depicted in the respective line in column 8 of Table IV, application no. 9, which is joined to a transit peptide; or
increasing or generating the activity of an expression product of one or more nucleic acid molecule(s) comprising a polynucleotide as depicted in the respective line in column 5 or 8 of Table I, application no. 9, preferably the coding region thereof, or a homolog or a fragment thereof, which is joined to a nucleic acid sequence encoding an organelle localization sequence, preferably a plastid or a mitochondrion localization sequence, especially a plastid localization sequence;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 9, or a homolog or a fragment thereof, a consensus sequence or at least one polypeptide motif as depicted in the respective line in column 8 of Table IV, application no. 9; or increasing or generating the activity of an expression product of one or more nucleic acid molecule(s) comprising a polynucleotide as depicted in the respective line in column 5 or 8 of Table I, application no. 9, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of linolenic acid, or a composition comprising linolenic acid in said non-human organism or in the culture medium surrounding said non-human organism.

Accordingly, the present invention relates to a process for the production of linolenic acid, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a At2g45420-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a sec-independent protein translocase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a acyl-CoA synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a ThiF family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a CTP synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a recombinase A in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a isopropylmalate isomerase large subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a HesB/YadR/YfhF family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a b0195-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a asparaginase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a multidrug resistance protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a 3-oxoacyl-[acyl-carrier-protein] reductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a 3-oxoacyl-[acyl-carrier-protein] synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a b1234-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a b1248-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a b1280-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a b1330-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a b1470-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a acid shock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a fumarate hydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a electron transport complex protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a b1670-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a b1672-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a b1837-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a b2032-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a b2107-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a b2399-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a ethanolamine utilization protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a b2474-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a b2613-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a b2682-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a flavodoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a AAS bifunctional protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a phosphotriesterase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a b3442-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a b3534-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a threonine efflux protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a b4023-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a branched chain amino acid ABC transporter ATP-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a phosphofructokinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a carbon dioxide concentrating mechanism protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a sll1032-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a CTP synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a carbamoyl-phosphate synthase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a acyl transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a coproporphyrinogen III oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a cation-transporting ATPase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a bifunctional purine biosynthesis protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a permease protein of ABC transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a aromatic acid decarboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a phosphate import ATP-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a arginine decarboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a phosphoadenosine phosphosulfate reductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a threonine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a metal-dependent hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a TTC1386-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a acid phosphatase precursor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a glycerol-3-phosphate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a cell division control protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a branched-chain amino acid permease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a F-box protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a ydr339c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a ydr507c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a dihydroxyacetone kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a DnaJ-like chaperone in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a ymr013c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a molecular chaperone in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a ATPase epsilon subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a glucose-6-phosphate 1-dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Accordingly, the present invention relates to a process for the production of linolenic acid, which comprises
(a) increasing or generating the activity of a polypeptide comprising a polypeptide as depicted in the respective line in column 5 or 8 of Table II, application no. 9, or a homolog or a fragment thereof, a consensus sequence or at least one polypeptide motif as depicted in the respective line in column 8 of Table IV, application no. 9; or
increasing or generating the activity of an expression product of one or more nucleic acid molecule(s) comprising a polynucleotide as depicted in the respective line in column 5 or 8 of Table I, application no. 9, preferably the coding region thereof, or a homolog or a fragment thereof;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of linolenic acid, or a composition comprising linolenic acid in said non-human organism or in the culture medium surrounding said non-human organism.

In a further embodiment the activity of the polypeptide comprising a polypep-tide as depicted in the respective line in column 5 or 8 of Table II, application no. 9, or a homolog or a fragment thereof, a consensus sequence or at least one polypeptide motif as depicted in the respective line in column 8 of Table IV, application no. 9, is increased or generated non-targeted in the above-mentioned process in a microorganism or plant or a part thereof.

In a further embodiment said polypeptide has the activity of the respective polypeptide represented by a protein comprising a polypeptide as depicted in the respective line in column 5 of Table II, application no. 9.

In a further embodiment the activity of the expression product of one or more nucleic acid molecule(s) comprising a polynucleotide as depicted in the respective line in column 5 or 8 of Table I, application no. 9, preferably the coding region thereof, or a homolog or a fragment thereof, is increased or generated non-targeted in the above-mentioned process in a microorganism or plant or a part thereof.

In a further embodiment the activity of the polypeptide comprising a polypeptide as depicted in the respective line in column 5 or 8 of Table II, application no. 9, or a homolog or a fragment thereof, a consensus sequence or at least one polypeptide motif as depicted in the respective line in column 8 of Table IV, application no. 9, is increased or generated in the above-mentioned process in an organelle preferably in plastids or mitochondria, especially in plastids, of a microorganism or plant.

In a further embodiment said polypeptide has the activity of the respective polypeptide represented by a protein comprising a polypeptide as depicted in the respective line in column 5 of Table II, application no. 9.

In a further embodiment the activity of the expression product of one or more nucleic acid molecule(s) comprising a polynucleotide as depicted in the respective line in column 5 or 8 of Table I, application no. 9, preferably the coding region thereof, or a homolog or a fragment thereof, is increased or generated in the above-mentioned process in an organelle, preferably in plasids or mitochondria, especially in plastids, of a microorganism or plant.

In a further embodiment the activity of the polypeptide comprising a polypeptide as depicted in the respective line in column 5 or 8 of Table II, application no. 9, or a homolog or a fragment thereof, a consensus sequence or at least one polypeptide motif as depicted in the respective line in column 8 of Table IV, application no. 9, is increased or generated in the above-mentioned process in the cyctosol of a cell, of a microorganism or plant.

In a further embodiment said polypeptide has the activity of the respective polypeptide represented by a protein comprising a polypeptide as depicted in the respective line in column 5 of Table II, application no. 9.

In a further embodiment the activity of the expression product of one or more nucleic acid molecule(s) comprising a polynucleotide as depicted in the respective line in column 5 or 8 of Table I, application no. 9, preferably the coding region thereof, or a homolog or a fragment thereof, is increased or generated in the above-mentioned process in the cytosol of a cell, of a microorganism or plant.

[0039.1.8.9] to [0066.1.8.9] for the disclosure of these paragraphs see [0039.1.8.8] to [0066.1.8.8] above.

As mentioned above the nucleic acid sequences coding for the proteins as shown in Table II, application no. 9, column 5 or 8, or homologs or fragments thereof, are joined to a nucleic acid sequence encoding a plastidic transit peptide, e.g, if for the nucleic acid molecule in Table I, column 6 the term “plastidic” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in Table II, application no. 9, column 5 or 8, or homologs or fragments thereof, e.g., if for the nucleic acid molecule in Table I, column 6 the term “plastidic” is indicated.

[0068.1.8.9] to [0072.1.8.9] for the disclosure of these paragraphs see [0068.1.8.8] to [0072.1.8.8] above.

As mentioned above the nucleic acid sequences coding for the proteins as shown in the respective line in Table II, application no. 9, column 5 or 8, or homologs or fragments thereof, are joined to a nucleic acid sequence encoding a mitochondric transit peptide, e.g., if for the respective nucleic acid molecule in Table I, column 6 the term “mitochondrial” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in the respective line in Table II, application no. 9, column 5 or 8, or homologs or fragments thereof, e.g., if for the nucleic acid molecule in the respective line in Table I, column 6 the term “mitochondrial” is indicated. (For the avoidance of doubt, the term “mitochondric” and “mitochondrial” are interchangeable.)

[0074.1.8.9] to [0075.1.8.9] for the disclosure of these paragraphs see [0074.1.8.8] to [0075.1.8.8] above.

Nucleic acid sequences coding for the transit peptides may be chemically synthesized either in part or wholly according to structure of transit peptide sequences disclosed in the prior art. Said natural or chemically synthesized sequences can be directly linked to the sequences encoding the mature protein or via a linker nucleic acid sequence, which may be typically less than 500 base pairs, preferably less than 450, 400, 350, 300, 250 or 200 base pairs, more preferably less than 150, 100, 90, 80, 70, 60, 50, 40 or 30 base pairs and most preferably less than 25, 20, 15, 12, 9, 6 or 3 base pairs in length and are in frame to the coding sequence.

Furthermore favorable nucleic acid sequences encoding transit peptides may comprise sequences derived from more than one biological and/or chemical source and may include a nucleic acid sequence derived from the amino-terminal region of the mature protein, which in its native state is linked to the transit peptide. In a preferred embodiment of the invention said amino-terminal region of the mature protein is typically less than 150 amino acids, preferably less than 140, 130, 120, 110, 100 or 90 amino acids, more preferably less than 80, 70, 60, 50, 40, 35, 30, 25 or 20 amino acids and most preferably less than 19, 18, 17, 16, 15, 14, 13, 12, 11 or 10 amino acids in length. But even shorter or longer stretches are also possible. In addition target sequences, which facilitate the transport of proteins to other cell compartments such as the vacuole, endoplasmic reticulum, golgi complex, glyoxysomes, peroxisomes, mitochondria (in case of a plastidal transit peptide) or plastids, especially chloroplasts (in case of a mitochondric transit peptide) may be also part of the inventive nucleic acid sequence. The proteins translated from said inventive nucleic acid sequences are a kind of fusion proteins that means the nucleic acid sequences encoding the plastidal transit peptide for example the ones shown in Table a, preferably the last one of the Table a are joint to the respective nucleic acid sequences shown in Table I, application no. 9, columns 5 or 8, especially the coding region thereof, or homologs or fragments thereof, in case of nucleic acid sequences being expressed in plastids, and that means the nucleic acid sequences encoding the mitochondric transit peptide for example the ones shown in Table b, preferably the last one of the Table b are joint to the respective nucleic acid sequences shown in Table I, application no. 9, columns 5 or 8, especially the coding region thereof, or homologs or fragments thereof, in case of nucleic acid sequences being expressed in mitochondria. The person skilled in the art is able to join said sequences in a functional manner. Advantageously the transit peptide part is cleaved off from the protein part shown in Table II, application no. 9, columns 5 or 8, or a homolog or fragment thereof, during or after the transport into the organelles, preferably into the plastids or mitochondria, respectively.

All products of the cleavage of the preferred transit peptide shown in the last line of Table a have preferably the N-terminal amino acid sequences QIA CSS or QIA EFQLTT in front of the start methionine of the respective protein mentioned in Table II, application no. 9, column 5 or 8. or homologs or fragments thereof. Other short amino acid sequences of an range of 1 to 20 amino acids preferable 2 to 15 amino acids, more preferable 3 to 10 amino acids most preferably 4 to 8 amino acids are also possible in front of the start methionine of the protein mentioned in Table II, application no. 9, columns 5 or 8, or homologs or fragments thereof. In case of the amino acid sequence QIA CSS the three amino acids in front of the start methionine are stemming from the LIC (=ligatation independent cloning) cassette. Said short amino acid sequence is preferred in the case of the expression of E. coli genes. In case of the amino acid sequence QIA EFQLTT the six amino acids in front of the start methionine are stemming from the LIC cassette. Said short amino acid sequence is preferred in the case of the expression of S. cerevisiae genes. The skilled worker knowns that other short sequences are also useful in the expression of the respective nucleic acid molecule comprising a polynucleotide as depicted in Table I, application no. 9, column 5 or 8, preferably the coding region thereof, or a homolog or a fragment thereof. Furthermore the skilled worker is aware of the fact that there is not a need for such short sequences in the expression of the genes.

[0077.1.8.9] to [0078.1.8.9] for the disclosure of these paragraphs see [0077.1.8.8] to [0078.1.8.8] above.

Alternatively to the targeting of the respective sequences shown in Table II, application no. 9, columns 5 or 8, or homologs or fragments thereof, preferably of sequences in general encoded in the nucleus with the aid of the targeting sequences mentioned for example in Table a alone, Table b alone, or in combination with other targeting sequences preferably into plastids or mitochondria, respectively, the nucleic acids of the invention can directly be introduced into the plastidal or mitochondrial genome. Therefore in a preferred embodiment the respective nucleic acid sequences shown in the respective line in Table I, application no. 9, columns 5 or 8, preferably the coding region thereof, or homologs or fragments thereof, are directly introduced and expressed in plastids e.g., if for the nucleic acid molecule in the respective line in Table I, column 6, the term “plastidic” is indicated, or are directly introduced and expressed in mitochondria e.g., if for the nucleic acid molecule in the respective line in Table I, column 6 the term “mitochondrial” is indicated.

The term “introduced” in the context of this specification shall mean the insertion of a nucleic acid sequence into the organism by means of a “transfection”, “transduction” or preferably by “transformation”.

A plastid, such as a chloroplast, or a mitochondrion, has been “transformed” by an exogenous (preferably foreign) nucleic acid sequence if a nucleic acid sequence has been introduced into the plastid or mitochondrion that means that this sequence has crossed the membrane or the membranes of the plastid or of the mitochondrion. The exogenous, preferably foreign, DNA may be integrated (covalently linked) into plastid or mitochondrial DNA making up the genome of the plastid or mitochondrion, or it may remain unintegrated (e.g., by including a chloroplast origin of replication). “Stably” integrated DNA sequences are those, which are inherited through plastid or mitochondrion replication, thereby transferring new plastids or mitochondria, with the features of the integrated DNA sequence to the progeny.

[0080.1.8.9] to [0083.1.8.9] for the disclosure of these paragraphs see [0080.1.8.8] to [0083.1.8.8] above.

For the process of the present invention it may be of great advantage that by transforming the plastids the intraspecies specific transgene flow is blocked, because a lot of species such as corn, cotton and rice have a strict maternal inheritance of plastids. By placing the respective genes specified in the respective line in Table I, application no. 9, columns 5 or 8, preferably the coding region, or homologs or fragments thereof, e.g. if for the nucleic acid molecule in the respective line in column 6 of Table I the term “plastidic” is indicated, in the plastids of plants, these genes will not be present in the pollen of said plants.

A further preferred embodiment of the invention relates to the use of so called “chloroplast localization sequences”, in which a first RNA sequence or molecule is capable of transporting or “chaperoning” a second RNA sequence, such as a RNA sequence transcribed from the sequences depicted in the respective line in Table I, application no. 9, columns 5 or 8, preferably the coding region thereof, or a homolog or a fragment thereof, or a sequence encoding a protein, as depicted in the respective line in Table II, application no. 9, columns 5 and 8, or a homolog or a fragment thereof, from an external environment inside a cell or outside a plastid into a chloroplast. In one embodiment the chloroplast localization signal is substantially similar or complementary to a complete or intact viroid sequence. The chloroplast localization signal may be encoded by a DNA sequence, which is transcribed into the chloroplast localization RNA. The term “viroid” refers to a naturally occurring single stranded RNA molecule (Flores, C. R. Acad. Sci. III. 324 (10), 943 (2001)). Viroids usually contain about 200-500 nucleotides and generally exist as circular molecules. Examples of viroids that contain chloroplast localization signals include but are not limited to ASBVd, PLMVd, CChMVd and ELVd. The viroid sequence or a functional part of it can be fused to the sequences depicted in the respective line in Table I, application no. 9, columns 5 or 8, preferably the coding region thereof, or a homolog or a fragment thereof, or a sequence encoding a protein, as depicted in the respective line in Table II, application no. 9, columns 5 or 8, or a homolog or a fragment thereof, in such a manner that the viroid sequence transports a sequence transcribed from a sequence as depicted in the respective line in Table I, application no. 9 columns 5 or 8, preferably the coding region thereof, or a homolog or a fragment thereof, or a sequence encoding a protein as depicted in the respective line in Table II, application no. 9 columns 5 or 8, or a homolog or a fragment thereof, into the chloroplasts, e.g. if for the nucleic acid molecule in column 6 of Table I the term “plastidic” is indicated. A preferred embodiment uses a modified ASBVd (Navarro et al., Virology. 268 (1), 218 (2000)).

In a further specific embodiment the protein to be expressed in the plastids such as the proteins depicted in the respective line in Table II, application no. 9, columns 5 or 8, or a homolog or a fragment thereof, e.g. if for the nucleic acid molecule in the respective line in column 6 of Table I the term “plastidic” is indicated, are encoded by different nucleic acids. Such a method is disclosed in WO 2004/040973, which shall be incorporated by reference. WO 2004/040973 teaches a method, which relates to the translocation of an RNA corresponding to a gene or gene fragment into the chloroplast by means of a chloroplast localization sequence.

The genes, which should be expressed in the plant or plant cells, are split into nucleic acid fragments, which are introduced into different compartments in the plant e.g. the nucleus, the plastids and/or mitochondria. Additionally plant cells are described in which the chloroplast contains a ribozyme fused at one end to an RNA encoding a fragment of a protein used in the inventive process such that the ribozyme can trans-splice the translocated fusion RNA to the RNA encoding the gene fragment to form and as the case may be reunite the nucleic acid fragments to an intact mRNA encoding a functional protein for example as disclosed in the respective line in Table II, application no. 9, columns 5 or 8, or a homolog or a fragment thereof.

In a preferred embodiment of the invention the nucleic acid sequences as shown in the respective line in Table I, application no. 9, columns 5 or 8, preferably the coding region thereof, or homologs or fragments thereof, e.g. if for the nucleic acid molecule in the respective line in column 6 of Table I the term “plastidic” is indicated, used in the inventive process are transformed into plastids, which are metabolically active. Those plastids should preferably maintain at a high copy number in the plant or plant tissue of interest, most preferably the chloroplasts found in green plant tissues, such as leaves or cotyledons or in seeds.

For a good expression in the plastids the nucleic acid sequences as shown in the respective line in Table I, application no. 9, columns 5 or 8, preferably the coding region thereof, or homologs or fragments thereof, e.g. if for the nucleic acid molecule in the respective line in column 6 of Table I the term “plastidic” is indicated, are introduced into an expression cassette using preferably a promoter and terminater, which are active in plastids preferably a chloroplast promoter. Examples of such promoters include the psbA promoter from the gene from spinach or pea, the rbcL promoter, and the atpB promoter from corn.

[0089.1.8.9] to [0092.1.8.9] for the disclosure of these paragraphs see [0089.1.8.8] to [0092.1.8.8] above.

Advantageously the process for the production of the fine chemical linolenic acid leads to an enhanced production of the respective fine chemical. The terms “enhanced” or “increase” mean at least a 10%, 20%, 30%, 40% or 50%, preferably at least 60%, 70%, 80%, 90% or 100%, more preferably 150%, 200%, 300%, 400% or 500% higher production of the respective fine chemical linolenic acid in comparison to the wild-type as defined above, e.g. that means in comparison to a non-human organism without the aforementioned modification of the activity of a protein as shown in the respective line in Table II, application no. 9, column 5 or 8, or a fragment or a homolog thereof. The modification of the activity of a protein as shown in the respective line in Table II, application no. 9, column 5 or 8, or a homolog or a fragment thereof, or their combination can be achieved by joining the protein to a respective transit peptide, e.g. if for the respective encoding nucleic acid molecule in column 6 of Table I the term “plastidic” or “mitochondrial” is indicated.

Surprisingly it was found, that the transgenic expression of a protein as shown in the respective line in Table II, application no. 9, column 5, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, conferred a production of or an increase in linolenic acid,respectively, to the transgenic non-human organism as compared to a corresponding non-transformed wild type.

In one embodiment the transgenic expression of a protein as shown in the respective line in Table II, application no. 9, column 5 or 8, or a homolog or a fragment thereof, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, confers a production of or an increase in linolenic acid,respectively, to the transgenic non-human organism as compared to a corresponding non-transformed wild type.

Accordingly, in one embodiment, an increase of linolenic acid in a non-human organism, as compared to a corresponding non-transformed wild type non-human organism, is conferred in the process of the invention, if the activity of a polypeptide showing the activity of a protein phosphatase, or if the activity of the polypeptide At1g07430, preferably represented by SEQ ID NO. 17452, or a homolog or fragment thereof, or if the activity of a polypeptide encoded by a nucleic acid molecule comprising the nucleic acid SEQ ID NO. 17451, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 17451 or polypeptide SEQ ID NO. 17452, respectively, or a homolog or a fragment thereof, is increased or generated, or if the activity protein phosphatase is increased or generated in a non-human organism, like a microorganism or a plant cell, plant or part thereof, especially with non-targeted localization. For example, an increase of the linolenic acid of at least 1 percent, particularly in a range of 12 to 52-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a transcription factor, or if the activity of the polypeptide At1g68320, preferably represented by SEQ ID NO. 1062, or a homolog or fragment thereof, or if the activity of a polypeptide encoded by a nucleic acid molecule comprising the nucleic acid SEQ ID NO. 1061, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 1061 or polypeptide SEQ ID NO. 1062, respectively, or a homolog or a fragment thereof, is increased or generated, or if the activity transcription factor is increased or generated in a non-human organism, like a microorganism or a plant cell, plant or part thereof, especially with non-targeted localization. For example, an increase of the linolenic acid of at least 1 percent, particularly in a range of 15 to 74-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a protein phosphatase, or if the activity of the polypeptide At1g72770, preferably represented by SEQ ID NO. 19420, or a homolog or fragment thereof, or if the activity of a polypeptide encoded by a nucleic acid molecule comprising the nucleic acid SEQ ID NO. 19419, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 19419 or polypeptide SEQ ID NO. 19420, respectively, or a homolog or a fragment thereof, is increased or generated, or if the activity protein phosphatase is increased or generated in a non-human organism, like a microorganism or a plant cell, plant or part thereof, especially with non-targeted localization. For example, an increase of the linolenic acid of at least 1 percent, particularly in a range of 21 to 49-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a pyruvate kinase, or if the activity of the polypeptide At2g36580, preferably represented by SEQ ID NO. 113715, or a homolog or fragment thereof, or if the activity of a polypeptide encoded by a nucleic acid molecule comprising the nucleic acid SEQ ID NO. 113714, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 113714 or polypeptide SEQ ID NO. 113715, respectively, or a homolog or a fragment thereof, is increased or generated, or if the activity pyruvate kinase is increased or generated in a non-human organism, like a microorganism or a plant cell, plant or part thereof, especially with plastidic localization. For example, an increase of the linolenic acid of at least 1 percent, particularly in a range of 12 to 22-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a At2g45420-protein, or if the activity of the polypeptide At2g45420, preferably represented by SEQ ID NO. 1624, or a homolog or fragment thereof, or if the activity of a polypeptide encoded by a nucleic acid molecule comprising the nucleic acid SEQ ID NO. 1623, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 1623 or polypeptide SEQ ID NO. 1624, respectively, or a homolog or a fragment thereof, is increased or generated, or if the activity At2g45420-protein is increased or generated in a non-human organism, like a microorganism or a plant cell, plant or part thereof, especially with non-targeted localization. For example, an increase of the linolenic acid of at least 1 percent, particularly in a range of 13 to 27-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a heat shock transcription factor, or if the activity of the polypeptide At3g02990, preferably represented by SEQ ID NO. 68658, or a homolog or fragment thereof, or if the activity of a polypeptide encoded by a nucleic acid molecule comprising the nucleic acid SEQ ID NO. 68657, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 68657 or polypeptide SEQ ID NO. 68658, respectively, or a homolog or a fragment thereof, is increased or generated, or if the activity heat shock transcription factor is increased or generated in a non-human organism, like a microorganism or a plant cell, plant or part thereof, especially with non-targeted localization. For example, an increase of the linolenic acid of at least 1 percent, particularly in a range of 14 to 39-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization. For example, an increase of the linolenic acid of at least 1 percent, particularly in a range of 14 to 36-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a monothiol glutaredoxin, or if the activity of the polypeptide At4g15670, preferably represented by SEQ ID NO. 3280, or a homolog or fragment thereof, or if the activity of a polypeptide encoded by a nucleic acid molecule comprising the nucleic acid SEQ ID NO. 3279, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 3279 or polypeptide SEQ ID NO. 3280, respectively, or a homolog or a fragment thereof, is increased or generated, or if the activity monothiol glutaredoxin is increased or generated in a non-human organism, like a microorganism or a plant cell, plant or part thereof, especially with non-targeted localization. For example, an increase of the linolenic acid of at least 1 percent, particularly in a range of 13 to 67-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a heat shock transcription factor, or if the activity of the polypeptide At4g18880, preferably represented by SEQ ID NO. 24233, or a homolog or fragment thereof, or if the activity of a polypeptide encoded by a nucleic acid molecule comprising the nucleic acid SEQ ID NO. 24232, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 24232 or polypeptide SEQ ID NO. 24233, respectively, or a homolog or a fragment thereof, is increased or generated, or if the activity heat shock transcription factor is increased or generated in a non-human organism, like a microorganism or a plant cell, plant or part thereof, especially with non-targeted localization. For example, an increase of the linolenic acid of at least 1 percent, particularly in a range of 11 to 18-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a protein phosphatase, or if the activity of the polypeptide At4g26080, preferably represented by SEQ ID NO. 68850, or a homolog or fragment thereof, or if the activity of a polypeptide encoded by a nucleic acid molecule comprising the nucleic acid SEQ ID NO. 68849, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 68849 or polypeptide SEQ ID NO. 68850, respectively, or a homolog or a fragment thereof, is increased or generated, or if the activity protein phosphatase is increased or generated in a non-human organism, like a microorganism or a plant cell, plant or part thereof, especially with non-targeted localization. For example, an increase of the linolenic acid of at least 1 percent, particularly in a range of 14 to 32-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a glutaredoxin, or if the activity of the polypeptide At4g33040, preferably represented by SEQ ID NO. 4103, or a homolog or fragment thereof, or if the activity of a polypeptide encoded by a nucleic acid molecule comprising the nucleic acid SEQ ID NO. 4102, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 4102 or polypeptide SEQ ID NO. 4103, respectively, or a homolog or a fragment thereof, is increased or generated, or if the activity glutaredoxin is increased or generated in a non-human organism, like a microorganism or a plant cell, plant or part thereof, especially with non-targeted localization. For example, an increase of the linolenic acid of at least 1 percent, particularly in a range of 15 to 43-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a heat shock transcription factor, or if the activity of the polypeptide At5g03720, preferably represented by SEQ ID NO. 24439, or a homolog or fragment thereof, or if the activity of a polypeptide encoded by a nucleic acid molecule comprising the nucleic acid SEQ ID NO. 24438, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 24438 or polypeptide SEQ ID NO. 24439, respectively, or a homolog or a fragment thereof, is increased or generated, or if the activity heat shock transcription factor is increased or generated in a non-human organism, like a microorganism or a plant cell, plant or part thereof, especially with non-targeted localization. For example, an increase of the linolenic acid of at least 1 percent, particularly in a range of 13 to 35-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a monothiol glutaredoxin, or if the activity of the polypeptide At5g18600, preferably represented by SEQ ID NO. 4905, or a homolog or fragment thereof, or if the activity of a polypeptide encoded by a nucleic acid molecule comprising the nucleic acid SEQ ID NO. 4904, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 4904 or polypeptide SEQ ID NO. 4905, respectively, or a homolog or a fragment thereof, is increased or generated, or if the activity monothiol glutaredoxin is increased or generated in a non-human organism, like a microorganism or a plant cell, plant or part thereof, especially with non-targeted localization. For example, an increase of the linolenic acid of at least 1 percent, particularly in a range of 13 to 35-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a protein phosphatase, or if the activity of the polypeptide At5g57050, preferably represented by SEQ ID NO. 5319, or a homolog or fragment thereof, or if the activity of a polypeptide encoded by a nucleic acid molecule comprising the nucleic acid SEQ ID NO. 5318, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 5318 or polypeptide SEQ ID NO. 5319, respectively, or a homolog or a fragment thereof, is increased or generated, or if the activity protein phosphatase is increased or generated in a non-human organism, like a microorganism or a plant cell, plant or part thereof, especially with non-targeted localization. For example, an increase of the linolenic acid of at least 1 percent, particularly in a range of 15 to 40-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a protein phosphatase, or if the activity of the polypeptide At5g59220, preferably represented by SEQ ID NO. 25499, or a homolog or fragment thereof, or if the activity of a polypeptide encoded by a nucleic acid molecule comprising the nucleic acid SEQ ID NO. 25498, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 25498 or polypeptide SEQ ID NO. 25499, respectively, or a homolog or a fragment thereof, is increased or generated, or if the activity protein phosphatase is increased or generated in a non-human organism, like a microorganism or a plant cell, plant or part thereof, especially with non-targeted localization. For example, an increase of the linolenic acid of at least 1 percent, particularly in a range of 24 to 60-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a sec-independent protein translocase, or if the activity of the polypeptide AvinDRAFT_{—}1624, preferably represented by SEQ ID NO. 26197, or a homolog or fragment thereof, or if the activity of a polypeptide encoded by a nucleic acid molecule comprising the nucleic acid SEQ ID NO. 26196, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 26196 or polypeptide SEQ ID NO. 26197, respectively, or a homolog or a fragment thereof, is increased or generated, or if the activity sec-independent protein translocase is increased or generated in a non-human organism, like a microorganism or a plant cell, plant or part thereof, especially with non-targeted localization. For example, an increase of the linolenic acid of at least 1 percent, particularly in a range of 11 to 26-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a oxidoreductase, or if the activity of the polypeptide AvinDRAFT_{—}1788, preferably represented by SEQ ID NO. 114084, or a homolog or fragment thereof, or if the activity of a polypeptide encoded by a nucleic acid molecule comprising the nucleic acid SEQ ID NO. 114083, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 114083 or polypeptide SEQ ID NO. 114084, respectively, or a homolog or a fragment thereof, is increased or generated, or if the activity oxidoreductase is increased or generated in a non-human organism, like a microorganism or a plant cell, plant or part thereof, especially with non-targeted localization. For example, an increase of the linolenic acid of at least 1 percent, particularly in a range of 16 to 26-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a acyl-CoA synthase, or if the activity of the polypeptide AvinDRAFT_{—}2754, preferably represented by SEQ ID NO. 28041, or a homolog or fragment thereof, or if the activity of a polypeptide encoded by a nucleic acid molecule comprising the nucleic acid SEQ ID NO. 28040, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 28040 or polypeptide SEQ ID NO. 28041, respectively, or a homolog or a fragment thereof, is increased or generated, or if the activity acyl-CoA synthase is increased or generated in a non-human organism, like a microorganism or a plant cell, plant or part thereof, especially with non-targeted localization. For example, an increase of the linolenic acid of at least 1 percent, particularly in a range of 13 to 26-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a oxidoreductase, or if the activity of the polypeptide AvinDRAFT_{—}3253, preferably represented by SEQ ID NO. 29501, or a homolog or fragment thereof, or if the activity of a polypeptide encoded by a nucleic acid molecule comprising the nucleic acid SEQ ID NO. 29500, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 29500 or polypeptide SEQ ID NO. 29501, respectively, or a homolog or a fragment thereof, is increased or generated, or if the activity oxidoreductase is increased or generated in a non-human organism, like a microorganism or a plant cell, plant or part thereof, especially with non-targeted localization. For example, an increase of the linolenic acid of at least 1 percent, particularly in a range of 14 to 58-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a ThiF family protein, or if the activity of the polypeptide AvinDRAFT_{—}3577, preferably represented by SEQ ID NO. 111156, or a homolog or fragment thereof, or if the activity of a polypeptide encoded by a nucleic acid molecule comprising the nucleic acid SEQ ID NO. 111155, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 111155 or polypeptide SEQ ID NO. 111156, respectively, or a homolog or a fragment thereof, is increased or generated, or if the activity ThiF family protein is increased or generated in a non-human organism, like a microorganism or a plant cell, plant or part thereof, especially with non-targeted localization. For example, an increase of the linolenic acid of at least 1 percent, particularly in a range of 12 to 43-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a CTP synthase, or if the activity of the polypeptide AvinDRAFT_{—}3605, preferably represented by SEQ ID NO. 31027, or a homolog or fragment thereof, or if the activity of a polypeptide encoded by a nucleic acid molecule comprising the nucleic acid SEQ ID NO. 31026, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 31026 or polypeptide SEQ ID NO. 31027, respectively, or a homolog or a fragment thereof, is increased or generated, or if the activity CTP synthase is increased or generated in a non-human organism, like a microorganism or a plant cell, plant or part thereof, especially with non-targeted localization. For example, an increase of the linolenic acid of at least 1 percent, particularly in a range of 11 to 46-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a recombinase A, or if the activity of the polypeptide AvinDRAFT_{—}3629, preferably represented by SEQ ID NO. 114232, or a homolog or fragment thereof, or if the activity of a polypeptide encoded by a nucleic acid molecule comprising the nucleic acid SEQ ID NO. 114231, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 114231 or polypeptide SEQ ID NO. 114232, respectively, or a homolog or a fragment thereof, is increased or generated, or if the activity recombinase A is increased or generated in a non-human organism, like a microorganism or a plant cell, plant or part thereof, especially with non-targeted localization. For example, an increase of the linolenic acid of at least 1 percent, particularly in a range of 15 to 47-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a isopropylmalate isomerase large subunit, or if the activity of the polypeptide AvinDRAFT_{—}4847, preferably represented by SEQ ID NO. 102942, or a homolog or fragment thereof, or if the activity of a polypeptide encoded by a nucleic acid molecule comprising the nucleic acid SEQ ID NO. 102941, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 102941 or polypeptide SEQ ID NO. 102942, respectively, or a homolog or a fragment thereof, is increased or generated, or if the activity isopropylmalate isomerase large subunit is increased or generated in a non-human organism, like a microorganism or a plant cell, plant or part thereof, especially with non-targeted localization. For example, an increase of the linolenic acid of at least 1 percent, particularly in a range of 12 to 26-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a HesB/YadR/YfhF family protein, or if the activity of the polypeptide AvinDRAFT_{—}5467, preferably represented by SEQ ID NO. 32649, or a homolog or fragment thereof, or if the activity of a polypeptide encoded by a nucleic acid molecule comprising the nucleic acid SEQ ID NO. 32648, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 32648 or polypeptide SEQ ID NO. 32649, respectively, or a homolog or a fragment thereof, is increased or generated, or if the activity HesB/YadR/YfhF family protein is increased or generated in a non-human organism, like a microorganism or a plant cell, plant or part thereof, especially with non-targeted localization. For example, an increase of the linolenic acid of at least 1 percent, particularly in a range of 21 to 54-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a b0195-protein, or if the activity of the polypeptide B0195, preferably represented by SEQ ID NO. 118239, or a homolog or fragment thereof, or if the activity of a polypeptide encoded by a nucleic acid molecule comprising the nucleic acid SEQ ID NO. 118238, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 118238 or polypeptide SEQ ID NO. 118239, respectively, or a homolog or a fragment thereof, is increased or generated, or if the activity b0195-protein is increased or generated in a non-human organism, like a microorganism or a plant cell, plant or part thereof, especially with plastidic localization. For example, an increase of the linolenic acid of at least 1 percent, particularly in a range of 14 to 31-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a asparaginase, or if the activity of the polypeptide B0828, preferably represented by SEQ ID NO. 36300, or a homolog or fragment thereof, or if the activity of a polypeptide encoded by a nucleic acid molecule comprising the nucleic acid SEQ ID NO. 36299, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 36299 or polypeptide SEQ ID NO. 36300, respectively, or a homolog or a fragment thereof, is increased or generated, or if the activity asparaginase is increased or generated in a non-human organism, like a microorganism or a plant cell, plant or part thereof, especially with plastidic localization. For example, an increase of the linolenic acid of at least 1 percent, particularly in a range of 12 to 28-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a multidrug resistance protein, or if the activity of the polypeptide B1065, preferably represented by SEQ ID NO. 76033, or a homolog or fragment thereof, or if the activity of a polypeptide encoded by a nucleic acid molecule comprising the nucleic acid SEQ ID NO. 76032, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 76032 or polypeptide SEQ ID NO. 76033, respectively, or a homolog or a fragment thereof, is increased or generated, or if the activity multidrug resistance protein is increased or generated in a non-human organism, like a microorganism or a plant cell, plant or part thereof, especially with plastidic localization. For example, an increase of the linolenic acid of at least 1 percent, particularly in a range of 15 to 48-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a 3-oxoacyl-[acyl-carrier-protein] reductase, or if the activity of the polypeptide B1093, preferably represented by SEQ ID NO. 118372, or a homolog or fragment thereof, or if the activity of a polypeptide encoded by a nucleic acid molecule comprising the nucleic acid SEQ ID NO. 118371, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 118371 or polypeptide SEQ ID NO. 118372, respectively, or a homolog or a fragment thereof, is increased or generated, or if the activity 3-oxoacyl-[acyl-carrier-protein] reductase is increased or generated in a non-human organism, like a microorganism or a plant cell, plant or part thereof, especially with non-targeted localization. For example, an increase of the linolenic acid of at least 1 percent, particularly in a range of 11 to 25-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a 3-oxoacyl-[acyl-carrier-protein] synthase, or if the activity of the polypeptide B1095, preferably represented by SEQ ID NO. 114553, or a homolog or fragment thereof, or if the activity of a polypeptide encoded by a nucleic acid molecule comprising the nucleic acid SEQ ID NO. 114552, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 114552 or polypeptide SEQ ID NO. 114553, respectively, or a homolog or a fragment thereof, is increased or generated, or if the activity 3-oxoacyl-[acyl-carrier-protein] synthase is increased or generated in a non-human organism, like a microorganism or a plant cell, plant or part thereof, especially with plastidic localization. For example, an increase of the linolenic acid of at least 1 percent, particularly in a range of 12 to 47-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a b1234-protein, or if the activity of the polypeptide B1234, preferably represented by SEQ ID NO. 112112, or a homolog or fragment thereof, or if the activity of a polypeptide encoded by a nucleic acid molecule comprising the nucleic acid SEQ ID NO. 112111, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 112111 or polypeptide SEQ ID NO. 112112, respectively, or a homolog or a fragment thereof, is increased or generated, or if the activity b1234-protein is increased or generated in a non-human organism, like a microorganism or a plant cell, plant or part thereof, especially with non-targeted localization. For example, an increase of the linolenic acid of at least 1 percent, particularly in a range of 11 to 30-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a b1248-protein, or if the activity of the polypeptide B1248, preferably represented by SEQ ID NO. 119136, or a homolog or fragment thereof, or if the activity of a polypeptide encoded by a nucleic acid molecule comprising the nucleic acid SEQ ID NO. 119135, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 119135 or polypeptide SEQ ID NO. 119136, respectively, or a homolog or a fragment thereof, is increased or generated, or if the activity b1248-protein is increased or generated in a non-human organism, like a microorganism or a plant cell, plant or part thereof, especially with non-targeted localization. For example, an increase of the linolenic acid of at least 1 percent, particularly in a range of 12 to 19-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a b1280-protein, or if the activity of the polypeptide B1280, preferably represented by SEQ ID NO. 37574, or a homolog or fragment thereof, or if the activity of a polypeptide encoded by a nucleic acid molecule comprising the nucleic acid SEQ ID NO. 37573, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 37573 or polypeptide SEQ ID NO. 37574, respectively, or a homolog or a fragment thereof, is increased or generated, or if the activity b1280-protein is increased or generated in a non-human organism, like a microorganism or a plant cell, plant or part thereof, especially with non-targeted localization. For example, an increase of the linolenic acid of at least 1 percent, particularly in a range of 13 to 28-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a b1330-protein, or if the activity of the polypeptide B1330, preferably represented by SEQ ID NO. 38227, or a homolog or fragment thereof, or if the activity of a polypeptide encoded by a nucleic acid molecule comprising the nucleic acid SEQ ID NO. 38226, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 38226 or polypeptide SEQ ID NO. 38227, respectively, or a homolog or a fragment thereof, is increased or generated, or if the activity b1330-protein is increased or generated in a non-human organism, like a microorganism or a plant cell, plant or part thereof, especially with non-targeted localization. For example, an increase of the linolenic acid of at least 1 percent, particularly in a range of 15 to 29-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a b1470-protein, or if the activity of the polypeptide B1470, preferably represented by SEQ ID NO. 115057, or a homolog or fragment thereof, or if the activity of a polypeptide encoded by a nucleic acid molecule comprising the nucleic acid SEQ ID NO. 115056, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 115056 or polypeptide SEQ ID NO. 115057, respectively, or a homolog or a fragment thereof, is increased or generated, or if the activity b1470-protein is increased or generated in a non-human organism, like a microorganism or a plant cell, plant or part thereof, especially with non-targeted localization. For example, an increase of the linolenic acid of at least 1 percent, particularly in a range of 12 to 30-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a acid shock protein, or if the activity of the polypeptide B1597, preferably represented by SEQ ID NO. 38301, or a homolog or fragment thereof, or if the activity of a polypeptide encoded by a nucleic acid molecule comprising the nucleic acid SEQ ID NO. 38300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 38300 or polypeptide SEQ ID NO. 38301, respectively, or a homolog or a fragment thereof, is increased or generated, or if the activity acid shock protein is increased or generated in a non-human organism, like a microorganism or a plant cell, plant or part thereof, especially with non-targeted localization. For example, an increase of the linolenic acid of at least 1 percent, particularly in a range of 17 to 33-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a fumarate hydratase, or if the activity of the polypeptide B1611, preferably represented by SEQ ID NO. 115065, or a homolog or fragment thereof, or if the activity of a polypeptide encoded by a nucleic acid molecule comprising the nucleic acid SEQ ID NO. 115064, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 115064 or polypeptide SEQ ID NO. 115065, respectively, or a homolog or a fragment thereof, is increased or generated, or if the activity fumarate hydratase is increased or generated in a non-human organism, like a microorganism or a plant cell, plant or part thereof, especially with plastidic localization. For example, an increase of the linolenic acid of at least 1 percent, particularly in a range of 13 to 43-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a electron transport complex protein, or if the activity of the polypeptide B1627, preferably represented by SEQ ID NO. 38346, or a homolog or fragment thereof, or if the activity of a polypeptide encoded by a nucleic acid molecule comprising the nucleic acid SEQ ID NO. 38345, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 38345 or polypeptide SEQ ID NO. 38346, respectively, or a homolog or a fragment thereof, is increased or generated, or if the activity electron transport complex protein is increased or generated in a non-human organism, like a microorganism or a plant cell, plant or part thereof, especially with non-targeted localization. For example, an increase of the linolenic acid of at least 1 percent, particularly in a range of 11 to 19-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a b1670-protein, or if the activity of the polypeptide B1670, preferably represented by SEQ ID NO. 78754, or a homolog or fragment thereof, or if the activity of a polypeptide encoded by a nucleic acid molecule comprising the nucleic acid SEQ ID NO. 78753, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 78753 or polypeptide SEQ ID NO. 78754, respectively, or a homolog or a fragment thereof, is increased or generated, or if the activity b1670-protein is increased or generated in a non-human organism, like a microorganism or a plant cell, plant or part thereof, especially with non-targeted localization. For example, an increase of the linolenic acid of at least 1 percent, particularly in a range of 12 to 30-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a b1672-protein, or if the activity of the polypeptide B1672, preferably represented by SEQ ID NO. 78772, or a homolog or fragment thereof, or if the activity of a polypeptide encoded by a nucleic acid molecule comprising the nucleic acid SEQ ID NO. 78771, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 78771 or polypeptide SEQ ID NO. 78772, respectively, or a homolog or a fragment thereof, is increased or generated, or if the activity b1672-protein is increased or generated in a non-human organism, like a microorganism or a plant cell, plant or part thereof, especially with non-targeted localization. For example, an increase of the linolenic acid of at least 1 percent, particularly in a range of 12 to 26-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a b1837-protein, or if the activity of the polypeptide B1837, preferably represented by SEQ ID NO. 78954, or a homolog or fragment thereof, or if the activity of a polypeptide encoded by a nucleic acid molecule comprising the nucleic acid SEQ ID NO. 78953, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 78953 or polypeptide SEQ ID NO. 78954, respectively, or a homolog or a fragment thereof, is increased or generated, or if the activity b1837-protein is increased or generated in a non-human organism, like a microorganism or a plant cell, plant or part thereof, especially with non-targeted localization. For example, an increase of the linolenic acid of at least 1 percent, particularly in a range of 14 to 32-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a b2032-protein, or if the activity of the polypeptide B2032, preferably represented by SEQ ID NO. 8921, or a homolog or fragment thereof, or if the activity of a polypeptide encoded by a nucleic acid molecule comprising the nucleic acid SEQ ID NO. 8920, preferably the coding region thereof, or a homolog or fragment thereof, e.g. derived from Escherichia coli, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 8920 or polypeptide SEQ ID NO. 8921, respectively, or a homolog or a fragment thereof, is increased or generated, or if the activity b2032-protein is increased or generated in a non-human organism, like a microorganism or a plant cell, plant or part thereof, especially with non-targeted localization. For example, an increase of the linolenic acid of at least 1 percent, particularly in a range of 12 to 90-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a b2107-protein, or if the activity of the polypeptide B2107, preferably represented by SEQ ID NO. 39003, or a homolog or fragment thereof, or if the activity of a polypeptide encoded by a nucleic acid molecule comprising the nucleic acid SEQ ID NO. 39002, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 39002 or polypeptide SEQ ID NO. 39003, respectively, or a homolog or a fragment thereof, is increased or generated, or if the activity b2107-protein is increased or generated in a non-human organism, like a microorganism or a plant cell, plant or part thereof, especially with non-targeted localization. For example, an increase of the linolenic acid of at least 1 percent, particularly in a range of 13 to 31-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a b2399-protein, or if the activity of the polypeptide B2399, preferably represented by SEQ ID NO. 39238, or a homolog or fragment thereof, or if the activity of a polypeptide encoded by a nucleic acid molecule comprising the nucleic acid SEQ ID NO. 39237, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 39237 or polypeptide SEQ ID NO. 39238, respectively, or a homolog or a fragment thereof, is increased or generated, or if the activity b2399-protein is increased or generated in a non-human organism, like a microorganism or a plant cell, plant or part thereof, especially with non-targeted localization. For example, an increase of the linolenic acid of at least 1 percent, particularly in a range of 30 to 52-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a ethanolamine utilization protein, or if the activity of the polypeptide B2439, preferably represented by SEQ ID NO. 115629, or a homolog or fragment thereof, or if the activity of a polypeptide encoded by a nucleic acid molecule comprising the nucleic acid SEQ ID NO. 115628, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 115628 or polypeptide SEQ ID NO. 115629, respectively, or a homolog or a fragment thereof, is increased or generated, or if the activity ethanolamine utilization protein is increased or generated in a non-human organism, like a microorganism or a plant cell, plant or part thereof, especially with non-targeted localization. For example, an increase of the linolenic acid of at least 1 percent, particularly in a range of 14 to 33-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a b2474-protein, or if the activity of the polypeptide B2474, preferably represented by SEQ ID NO. 40330, or a homolog or fragment thereof, or if the activity of a polypeptide encoded by a nucleic acid molecule comprising the nucleic acid SEQ ID NO. 40329, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 40329 or polypeptide SEQ ID NO. 40330, respectively, or a homolog or a fragment thereof, is increased or generated, or if the activity b2474-protein is increased or generated in a non-human organism, like a microorganism or a plant cell, plant or part thereof, especially with non-targeted localization. For example, an increase of the linolenic acid of at least 1 percent, particularly in a range of 12 to 38-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a b2613-protein, or if the activity of the polypeptide B2613, preferably represented by SEQ ID NO. 40666, or a homolog or fragment thereof, or if the activity of a polypeptide encoded by a nucleic acid molecule comprising the nucleic acid SEQ ID NO. 40665, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 40665 or polypeptide SEQ ID NO. 40666, respectively, or a homolog or a fragment thereof, is increased or generated, or if the activity b2613-protein is increased or generated in a non-human organism, like a microorganism or a plant cell, plant or part thereof, especially with non-targeted localization. For example, an increase of the linolenic acid of at least 1 percent, particularly in a range of 13 to 50-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a b2682-protein, or if the activity of the polypeptide B2682, preferably represented by SEQ ID NO. 115656, or a homolog or fragment thereof, or if the activity of a polypeptide encoded by a nucleic acid molecule comprising the nucleic acid SEQ ID NO. 115655, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 115655 or polypeptide SEQ ID NO. 115656, respectively, or a homolog or a fragment thereof, is increased or generated, or if the activity b2682-protein is increased or generated in a non-human organism, like a microorganism or a plant cell, plant or part thereof, especially with non-targeted localization. For example, an increase of the linolenic acid of at least 1 percent, particularly in a range of 16 to 25-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a flavodoxin, or if the activity of the polypeptide B2790, preferably represented by SEQ ID NO. 115720, or a homolog or fragment thereof, or if the activity of a polypeptide encoded by a nucleic acid molecule comprising the nucleic acid SEQ ID NO. 115719, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 115719 or polypeptide SEQ ID NO. 115720, respectively, or a homolog or a fragment thereof, is increased or generated, or if the activity flavodoxin is increased or generated in a non-human organism, like a microorganism or a plant cell, plant or part thereof, especially with non-targeted localization. For example, an increase of the linolenic acid of at least 1 percent, particularly in a range of 18 to 31-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a AAS bifunctional protein, or if the activity of the polypeptide B2836, preferably represented by SEQ ID NO. 115792, or a homolog or fragment thereof, or if the activity of a polypeptide encoded by a nucleic acid molecule comprising the nucleic acid SEQ ID NO. 115791, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 115791 or polypeptide SEQ ID NO. 115792, respectively, or a homolog or a fragment thereof, is increased or generated, or if the activity AAS bifunctional protein is increased or generated in a non-human organism, like a microorganism or a plant cell, plant or part thereof, especially with plastidic localization. For example, an increase of the linolenic acid of at least 1 percent, particularly in a range of 14 to 38-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a phosphotriesterase, or if the activity of the polypeptide B3379, preferably represented by SEQ ID NO. 116111, or a homolog or fragment thereof, or if the activity of a polypeptide encoded by a nucleic acid molecule comprising the nucleic acid SEQ ID NO. 116110, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 116110 or polypeptide SEQ ID NO. 116111, respectively, or a homolog or a fragment thereof, is increased or generated, or if the activity phosphotriesterase is increased or generated in a non-human organism, like a microorganism or a plant cell, plant or part thereof, especially with non-targeted localization. For example, an increase of the linolenic acid of at least 1 percent, particularly in a range of 11 to 26-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a b3442-protein, or if the activity of the polypeptide B3442, preferably represented by SEQ ID NO. 94381, or a homolog or fragment thereof, or if the activity of a polypeptide encoded by a nucleic acid molecule comprising the nucleic acid SEQ ID NO. 94380, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 94380 or polypeptide SEQ ID NO. 94381, respectively, or a homolog or a fragment thereof, is increased or generated, or if the activity b3442-protein is increased or generated in a non-human organism, like a microorganism or a plant cell, plant or part thereof, especially with non-targeted localization. For example, an increase of the linolenic acid of at least 1 percent, particularly in a range of 12 to 33-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a b3534-protein, or if the activity of the polypeptide B3534, preferably represented by SEQ ID NO. 119182, or a homolog or fragment thereof, or if the activity of a polypeptide encoded by a nucleic acid molecule comprising the nucleic acid SEQ ID NO. 119181, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 119181 or polypeptide SEQ ID NO. 119182, respectively, or a homolog or a fragment thereof, is increased or generated, or if the activity b3534-protein is increased or generated in a non-human organism, like a microorganism or a plant cell, plant or part thereof, especially with plastidic localization. For ex-ample, an increase of the linolenic acid of at least 1 percent, particularly in a range of 12 to 29-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a threonine efflux protein, or if the activity of the polypeptide B3823, preferably represented by SEQ ID NO. 10727, or a homolog or fragment thereof, or if the activity of a polypeptide encoded by a nucleic acid molecule comprising the nucleic acid SEQ ID NO. 10726, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 10726 or polypeptide SEQ ID NO. 10727, respectively, or a homolog or a fragment thereof, is increased or generated, or if the activity threonine efflux protein is increased or generated in a non-human organism, like a microorganism or a plant cell, plant or part thereof, especially with non-targeted localization. For example, an increase of the linolenic acid of at least 1 percent, particularly in a range of 11 to 29-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a b4023-protein, or if the activity of the polypeptide B4023, preferably represented by SEQ ID NO. 119210, or a homolog or fragment thereof, or if the activity of a polypeptide encoded by a nucleic acid molecule comprising the nucleic acid SEQ ID NO. 119209, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 119209 or polypeptide SEQ ID NO. 119210, respectively, or a homolog or a fragment thereof, is increased or generated, or if the activity b4023-protein is increased or generated in a non-human organism, like a microorganism or a plant cell, plant or part thereof, especially with non-targeted localization. For example, an increase of the linolenic acid of at least 1 percent, particularly in a range of 13 to 24-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a glutaredoxin, or if the activity of the polypeptide GM02LC12622, preferably represented by SEQ ID NO. 10812, or a homolog or fragment thereof, or if the activity of a polypeptide encoded by a nucleic acid molecule comprising the nucleic acid SEQ ID NO. 10811, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 10811 or polypeptide SEQ ID NO. 10812, respectively, or a homolog or a fragment thereof, is increased or generated, or if the activity glutaredoxin is increased or generated in a non-human organism, like a microorganism or a plant cell, plant or part thereof, especially with non-targeted localization. For example, an increase of the linolenic acid of at least 1 percent, particularly in a range of 13 to 42-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a transcription factor, or if the activity of the polypeptide GM02LC19289, preferably represented by SEQ ID NO. 46752, or a homolog or fragment thereof, or if the activity of a polypeptide encoded by a nucleic acid molecule comprising the nucleic acid SEQ ID NO. 46751, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 46751 or polypeptide SEQ ID NO. 46752, respectively, or a homolog or a fragment thereof, is increased or generated, or if the activity transcription factor is increased or generated in a non-human organism, like a microorganism or a plant cell, plant or part thereof, especially with non-targeted localization. For example, an increase of the linolenic acid of at least 1 percent, particularly in a range of 16 to 89-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a branched chain amino acid ABC transporter ATP-binding protein, or if the activity of the polypeptide Sll0374, preferably represented by SEQ ID NO. 94742, or a homolog or fragment thereof, or if the activity of a polypeptide encoded by a nucleic acid molecule comprising the nucleic acid SEQ ID NO. 94741, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table 1, 11 or IV, application No. 9, column 5 or 8 in the respective same line as the nucleic acid molecule SEQ ID NO. 94741 or polypeptide SEQ ID NO. 94742, respectively, or a homolog or a fragment thereof, is increased or generated, or if the activity branched chain amino acid ABC transporter ATP-binding protein is increased or generated in a non-human organism, like a microorganism or a plant cell, plant or part thereof, especially with plastidic localization. For example, an increase of the linolenic acid of at least 1 percent, particularly in a range of 15 to 18-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a phosphofructokinase, or if the activity of the polypeptide Sll0745, preferably represented by SEQ ID NO. 112662, or a homolog or fragment thereof, or if the activity of a polypeptide encoded by a nucleic acid molecule comprising the nucleic acid SEQ ID NO. 112661, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 112661 or polypeptide SEQ ID NO. 112662, respectively, or a homolog or a fragment thereof, is increased or generated, or if the activity phosphofructokinase is increased or generated in a non-human organism, like a microorganism or a plant cell, plant or part thereof, especially with mitochondrial localization. For example, an increase of the linolenic acid of at least 1 percent, particularly in a range of 13 to 29-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a carbon dioxide concentrating mechanism protein, or if the activity of the polypeptide Sll1031, preferably represented by SEQ ID NO. 52635, or a homolog or fragment thereof, or if the activity of a polypeptide encoded by a nucleic acid molecule comprising the nucleic acid SEQ ID NO. 52634, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table 1, 11 or IV, application No. 9, column 5 or 8 in the respective same line as the nucleic acid molecule SEQ ID NO. 52634 or polypeptide SEQ ID NO. 52635, respectively, or a homolog or a fragment thereof, is increased or generated, or if the activity carbon dioxide concentrating mechanism protein is increased or generated in a non-human organism, like a microorganism or a plant cell, plant or part thereof, especially with mitochondrial localization. For example, an increase of the linolenic acid of at least 1 percent, particularly in a range of 12 to 46-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a sll1032-protein, or if the activity of the polypeptide Sll1032, preferably represented by SEQ ID NO. 85724, or a homolog or fragment thereof, or if the activity of a polypeptide encoded by a nucleic acid molecule comprising the nucleic acid SEQ ID NO. 85723, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, 11 or IV, application No. 9, column 5 or 8 in the respective same line as the nucleic acid molecule SEQ ID NO. 85723 or polypeptide SEQ ID NO. 85724, respectively, or a homolog or a fragment thereof, is increased or generated, or if the activity sll1032-protein is increased or generated in a non-human organism, like a microorganism or a plant cell, plant or part thereof, especially with mitochondrial localization. For example, an increase of the linolenic acid of at least 1 percent, particularly in a range of 12 to 30-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a CTP synthetase, or if the activity of the poylpeptide Sll1443, preferably represented by SEQ ID NO. 53879, or a homolog or fragment thereof, or if the activity of a polypeptide encoded by a nucleic acid molecule comprising the nucleic acid SEQ ID NO. 53878, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 53878 or polypeptide SEQ ID NO. 53879, respectively, or a homolog or a fragment thereof, is increased or generated, or if the activity CTP synthetase is increased or generated in a non-human organism, like a microorganism or a plant cell, plant or part thereof, especially with mitochondrial localization. For example, an increase of the linolenic acid of at least 1 percent, particularly in a range of 14 to 33-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a carbamoylphosphate synthase subunit, or if the activity of the polypeptide Sll1498, preferably represented by SEQ ID NO. 95048, or a homolog or fragment thereof, or if the activity of a polypeptide encoded by a nucleic acid molecule comprising the nucleic acid SEQ ID NO. 95047, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, 11 or IV, application No. 9, column 5 or 8 in the respective same line as the nucleic acid molecule SEQ ID NO. 95047 or polypeptide SEQ ID NO. 95048, respectively, or a homolog or a fragment thereof, is increased or generated, or if the activity carbamoyl-phosphate synthase subunit is increased or generated in a non-human organism, like a microorganism or a plant cell, plant or part thereof, especially with non-targeted localization. For example, an increase of the linolenic acid of at least 1 percent, particularly in a range of 14 to 30-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a acyl transferase, or if the activity of the polypeptide Sll1848, preferably represented by SEQ ID NO. 86448, or a homolog or fragment thereof, or if the activity of a polypeptide encoded by a nucleic acid molecule comprising the nucleic acid SEQ ID NO. 86447, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 86447 or polypeptide SEQ ID NO. 86448, respectively, or a homolog or a fragment thereof, is increased or generated, or if the activity acyl transferase is increased or generated in a non-human organism, like a microorganism or a plant cell, plant or part thereof, especially with plastidic localization. For example, an increase of the linolenic acid of at least 1 percent, particularly in a range of 17 to 23-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a coproporphyrinogen III oxidase, or if the activity of the poylpeptide Sll1917, preferably represented by SEQ ID NO. 11472, or a homolog or fragment thereof, or if the activity of a polypeptide encoded by a nucleic acid molecule comprising the nucleic acid SEQ ID NO. 11471, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, 11 or IV, application No. 9, column 5 or 8 in the respective same line as the nucleic acid molecule SEQ ID NO. 11471 or polypeptide SEQ ID NO. 11472, respectively, or a homolog or a fragment thereof, is increased or generated, or if the activity coproporphyrinogen III oxidase is increased or generated in a non-human organism, like a microorganism or a plant cell, plant or part thereof, especially with mitochondrial localization. For example, an increase of the linolenic acid of at least 1 percent, particularly in a range of 12 to 24-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a cation-transporting ATPase, or if the activity of the polypeptide Sll1920, preferably represented by SEQ ID NO. 55386, or a homolog or fragment thereof, or if the activity of a polypeptide encoded by a nucleic acid molecule comprising the nucleic acid SEQ ID NO. 55385, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table 1, 11 or IV, application No. 9, column 5 or 8 in the respective same line as the nucleic acid molecule SEQ ID NO. 55385 or polypeptide SEQ ID NO. 55386, respectively, or a homolog or a fragment thereof, is increased or generated, or if the activity cation-transporting ATPase is increased or generated in a non-human organism, like a microorganism or a plant cell, plant or part thereof, especially with mitochondrial localization. For example, an increase of the linolenic acid of at least 1 percent, particularly in a range of 14 to 34-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a oxidoreductase, or if the activity of the polypeptide Slr0338, preferably represented by SEQ ID NO. 11991, or a homolog or fragment thereof, or if the activity of a polypeptide encoded by a nucleic acid molecule comprising the nucleic acid SEQ ID NO. 11990, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 11990 or polypeptide SEQ ID NO. 11991, respectively, or a homolog or a fragment thereof, is increased or generated, or if the activity oxidoreductase is increased or generated in a non-human organism, like a microorganism or a plant cell, plant or part thereof, especially with non-targeted localization. For example, an increase of the linolenic acid of at least 1 percent, particularly in a range of 12 to 25-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a bifunctional purine biosynthesis protein, or if the activity of the polypeptide Slr0597, preferably represented by SEQ ID NO. 56154, or a homolog or fragment thereof, or if the activity of a polypeptide encoded by a nucleic acid molecule comprising the nucleic acid SEQ ID NO. 56153, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 56153 or polypeptide SEQ ID NO. 56154, respectively, or a homolog or a fragment thereof, is increased or generated, or if the activity bifunctional purine biosynthesis protein is increased or generated in a non-human organism, like a microorganism or a plant cell, plant or part thereof, especially with plastidic localization. For example, an increase of the linolenic acid of at least 1 percent, particularly in a range of 13 to 36-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a permease protein of ABC transporter, or if the activity of the polypeptide Slr0949, preferably represented by SEQ ID NO. 119223, or a homolog or fragment thereof, or if the activity of a polypeptide encoded by a nucleic acid molecule comprising the nucleic acid SEQ ID NO. 119222, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the con-sensus sequence or the polypeptide motif, as depicted in Table I, II or IV, application No. 9, column 5 or 8 in the respective same line as the nucleic acid molecule SEQ ID NO. 119222 or polypeptide SEQ ID NO. 119223, respectively, or a homolog or a fragment thereof, is increased or generated, or if the activity permease protein of ABC transporter is increased or generated in a non-human organism, like a microorganism or a plant cell, plant or part thereof, especially with mitochondrial localization. For example, an increase of the linolenic acid of at least 1 percent, particularly in a range of 13 to 26-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a aromatic acid decarboxylase, or if the activity of the polypeptide Slr1099, preferably represented by SEQ ID NO. 116342, or a homolog or fragment thereof, or if the activity of a polypeptide encoded by a nucleic acid molecule comprising the nucleic acid SEQ ID NO. 116341, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 116341 or polypeptide SEQ ID NO. 116342, respectively, or a homolog or a fragment thereof, is increased or generated, or if the activity aromatic acid decarboxylase is increased or generated in a non-human organism, like a microorganism or a plant cell, plant or part thereof, especially with mitochondrial localization. For example, an increase of the linolenic acid of at least 1 percent, particularly in a range of 12 to 41-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a phosphate import ATP-binding protein, or if the activity of the polypeptide Slr1250, preferably represented by SEQ ID NO. 116461, or a homolog or fragment thereof, or if the activity of a polypeptide encoded by a nucleic acid molecule comprising the nucleic acid SEQ ID NO. 116460, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the con-sensus sequence or the polypeptide motif, as depicted in Table I, II or IV, application No. 9, column 5 or 8 in the respective same line as the nucleic acid molecule SEQ ID NO. 116460 or polypeptide SEQ ID NO. 116461, respectively, or a homolog or a fragment thereof, is increased or generated, or if the activity phosphate import ATP-binding protein is increased or generated in a non-human organism, like a microorganism or a plant cell, plant or part thereof, especially with mitochondrial localization. For example, an increase of the linolenic acid of at least 1 percent, particularly in a range of 13 to 26-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a arginine decarboxylase, or if the activity of the polypeptide Slr1312, preferably represented by SEQ ID NO. 58325, or a homolog or fragment thereof, or if the activity of a polypeptide encoded by a nucleic acid molecule comprising the nucleic acid SEQ ID NO. 58324, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 58324 or polypeptide SEQ ID NO. 58325, respectively, or a homolog or a fragment thereof, is increased or generated, or if the activity arginine decarboxylase is increased or generated in a non-human organism, like a microorganism or a plant cell, plant or part thereof, especially with non-targeted localization. For example, an increase of the linolenic acid of at least 1 percent, particularly in a range of 11 to 19-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a phosphoadenosine phosphosulfate reductase , or if the activity of the polypeptide Slr1791, preferably represented by SEQ ID NO. 12141, or a homolog or fragment thereof, or if the activity of a polypeptide encoded by a nucleic acid molecule comprising the nucleic acid SEQ ID NO. 12140, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 12140 or polypeptide SEQ ID NO. 12141, respectively, or a homolog or a fragment thereof, is increased or generated, or if the activity phosphoadenosine phosphosulfate reductase is increased or generated in a non-human organism, like a microorganism or a plant cell, plant or part thereof, especially with mitochondrial localization. For example, an increase of the linolenic acid of at least 1 percent, particularly in a range of 12 to 25-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a threonine dehydratase, or if the activity of the polypeptide Slr2072, preferably represented by SEQ ID NO. 12342, or a homolog or fragment thereof, or if the activity of a polypeptide encoded by a nucleic acid molecule comprising the nucleic acid SEQ ID NO. 12341, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 12341 or polypeptide SEQ ID NO. 12342, respectively, or a homolog or a fragment thereof, is increased or generated, or if the activity threonine dehydratase is increased or generated in a non-human organism, like a microorganism or a plant cell, plant or part thereof, especially with mitochondrial localization. For example, an increase of the linolenic acid of at least 1 percent, particularly in a range of 18 to 43-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a metal-dependent hydrolase, or if the activity of the polypeptide TTC0917, preferably represented by SEQ ID NO. 61554, or a homolog or fragment thereof, or if the activity of a polypeptide encoded by a nucleic acid molecule comprising the nucleic acid SEQ ID NO. 61553, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consen-sus sequence or the polypeptide motif, as depicted in Table I, II or IV, application No. 9, column 5 or 8 in the respective same line as the nucleic acid molecule SEQ ID NO. 61553 or polypeptide SEQ ID NO. 61554, respectively, or a homolog or a fragment thereof, is increased or generated, or if the activity metal-dependent hydrolase is increased or generated in a non-human organism, like a microorganism or a plant cell, plant or part thereof, especially with non-targeted localization. For example, an increase of the linolenic acid of at least 1 percent, particularly in a range of 15 to 105-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a TTC1386-protein, or if the activity of the polypeptide TTC1386, preferably represented by SEQ ID NO. 62080, or a homolog or fragment thereof, or if the activity of a polypeptide encoded by a nucleic acid molecule comprising the nucleic acid SEQ ID NO. 62079, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 62079 or polypeptide SEQ ID NO. 62080, respectively, or a homolog or a fragment thereof, is increased or generated, or if the activity TTC1386-protein is increased or generated in a non-human organism, like a microorganism or a plant cell, plant or part thereof, especially with non-targeted localization. For example, an increase of the linolenic acid of at least 1 percent, particularly in a range of 16 to 24-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a acid phosphatase precursor, or if the activity of the polypeptide Ybr092c, preferably represented by SEQ ID NO. 119348, or a homolog or fragment thereof, or if the activity of a polypeptide encoded by a nucleic acid molecule comprising the nucleic acid SEQ ID NO. 119347, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 119347 or polypeptide SEQ ID NO. 119348, respectively, or a homolog or a fragment thereof, is increased or generated, or if the activity acid phosphatase precursor is increased or generated in a non-human organism, like a microorganism or a plant cell, plant or part thereof, especially with plastidic localization. For example, an increase of the linolenic acid of at least 1 percent, particularly in a range of 12 to 25-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a glycerol-3-phosphate dehydrogenase, or if the activity of the polypeptide Ydl022w, preferably represented by SEQ ID NO. 119409, or a homolog or fragment thereof, or if the activity of a polypeptide encoded by a nucleic acid molecule comprising the nucleic acid SEQ ID NO. 119408, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 119408 or polypeptide SEQ ID NO. 119409, respectively, or a homolog or a fragment thereof, is increased or generated, or if the activity glycerol-3-phosphate dehydrogenase is increased or generated in a non-human organism, like a microorganism or a plant cell, plant or part thereof, especially with non-targeted localization. For example, an increase of the linolenic acid of at least 1 percent, particularly in a range of 13 to 34-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a cell division control protein, or if the activity of the polypeptide Yd1126c, preferably represented by SEQ ID NO. 120046, or a homolog or fragment thereof, or if the activity of a polypeptide encoded by a nucleic acid molecule comprising the nucleic acid SEQ ID NO. 120045, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table 1, 11 or IV, application No. 9, column 5 or 8 in the respective same line as the nucleic acid molecule SEQ ID NO. 120045 or polypeptide SEQ ID NO. 120046, respectively, or a homolog or a fragment thereof, is increased or generated, or if the activity cell division control protein is increased or generated in a non-human organism, like a microorganism or a plant cell, plant or part thereof, especially with non-targeted localization. For example, an increase of the linolenic acid of at least 1 percent, particularly in a range of 16 to 16-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a branched-chain amino acid permease, or if the activity of the polypeptide Ydr046c, preferably represented by SEQ ID NO. 63545, or a homolog or fragment thereof, or if the activity of a polypeptide encoded by a nucleic acid molecule comprising the nucleic acid SEQ ID NO. 63544, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 63544 or polypeptide SEQ ID NO. 63545, respectively, or a homolog or a fragment thereof, is increased or generated, or if the activity branched-chain amino acid permease is increased or generated in a non-human organism, like a microorganism or a plant cell, plant or part thereof, especially with plastidic localization. For example, an increase of the linolenic acid of at least 1 percent, particularly in a range of 17 to 22-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a F-box protein, or if the activity of the polypeptide Ydr131c, preferably represented by SEQ ID NO. 14707, or a homolog or fragment thereof, or if the activity of a polypeptide encoded by a nucleic acid molecule comprising the nucleic acid SEQ ID NO. 14706, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 14706 or polypeptide SEQ ID NO. 14707, respectively, or a homolog or a fragment thereof, is increased or generated, or if the activity F-box protein is increased or generated in a non-human organism, like a microorganism or a plant cell, plant or part thereof, especially with plastidic localization. For example, an increase of the linolenic acid of at least 1 percent, particularly in a range of 19 to 38-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a ydr339c-protein, or if the activity of the polypeptide Ydr339c, preferably represented by SEQ ID NO. 120242, or a homolog or fragment thereof, or if the activity of a polypeptide encoded by a nucleic acid molecule comprising the nucleic acid SEQ ID NO. 120241, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 120241 or polypeptide SEQ ID NO. 120242, respectively, or a homolog or a fragment thereof, is increased or generated, or if the activity ydr339c-protein is increased or generated in a non-human organism, like a microorganism or a plant cell, plant or part thereof, especially with plastidic localization. For example, an increase of the linolenic acid of at least 1 percent, particularly in a range of 15 to 18-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a ydr507c-protein, or if the activity of the polypeptide Ydr507c, preferably represented by SEQ ID NO. 90166, or a homolog or fragment thereof, or if the activity of a polypeptide encoded by a nucleic acid molecule comprising the nucleic acid SEQ ID NO. 90165, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 90165 or polypeptide SEQ ID NO. 90166, respectively, or a homolog or a fragment thereof, is increased or generated, or if the activity ydr507c-protein is increased or generated in a non-human organism, like a microorganism or a plant cell, plant or part thereof, especially with non-targeted localization. For example, an increase of the linolenic acid of at least 1 percent, particularly in a range of 11 to 45-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a dihydroxyacetone kinase, or if the activity of the polypeptide Yf1053w, preferably represented by SEQ ID NO. 120310, or a homolog or fragment thereof, or if the activity of a polypeptide encoded by a nucleic acid molecule comprising the nucleic acid SEQ ID NO. 120309, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 120309 or polypeptide SEQ ID NO. 120310, respectively, or a homolog or a fragment thereof, is increased or generated, or if the activity dihydroxyacetone kinase is increased or generated in a non-human organism, like a microorganism or a plant cell, plant or part thereof, especially with non-targeted localization. For example, an increase of the linolenic acid of at least 1 percent, particularly in a range of 11 to 41-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a oxidoreductase, or if the activity of the polypeptide Ygl039w, preferably represented by SEQ ID NO. 120437, or a homolog or fragment thereof, or if the activity of a polypeptide encoded by a nucleic acid molecule comprising the nucleic acid SEQ ID NO. 120436, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, 11 or IV, application No. 9, column 5 or 8 in the respective same line as the nucleic acid molecule SEQ ID NO. 120436 or polypeptide SEQ ID NO. 120437, respectively, or a homolog or a fragment thereof, is increased or generated, or if the activity oxidoreductase is increased or generated in a non-human organism, like a microorganism or a plant cell, plant or part thereof, especially with plastidic localization. For example, an increase of the linolenic acid of at least 1 percent, particularly in a range of 17 to 26-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a DnaJ-like chaperone, or if the activity of the polypeptide Yj1073w, preferably represented by SEQ ID NO. 64965, or a homolog or fragment thereof, or if the activity of a polypeptide encoded by a nucleic acid molecule comprising the nucleic acid SEQ ID NO. 64964, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 64964 or polypeptide SEQ ID NO. 64965, respectively, or a homolog or a fragment thereof, is increased or generated, or if the activity DnaJ-like chaperone is increased or generated in a non-human organism, like a microorganism or a plant cell, plant or part thereof, especially with plastidic localization. For example, an increase of the linolenic acid of at least 1 percent, particularly in a range of 15 to 27-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a ymr013c-protein, or if the activity of the polypeptide Ymr013c, preferably represented by SEQ ID NO. 91959, or a homolog or fragment thereof, or if the activity of a polypeptide encoded by a nucleic acid molecule comprising the nucleic acid SEQ ID NO. 91958, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 91958 or polypeptide SEQ ID NO. 91959, respectively, or a homolog or a fragment thereof, is increased or generated, or if the activity ymr013c-protein is increased or generated in a non-human organism, like a microorganism or a plant cell, plant or part thereof, especially with plastidic localization. For example, an increase of the linolenic acid of at least 1 percent, particularly in a range of 13 to 17-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a molecular chaperone, or if the activity of the polypeptide Ynl064c, preferably represented by SEQ ID NO. 117496, or a homolog or fragment thereof, or if the activity of a polypeptide encoded by a nucleic acid molecule comprising the nucleic acid SEQ ID NO. 117495, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 117495 or polypeptide SEQ ID NO. 117496, respectively, or a homolog or a fragment thereof, is increased or generated, or if the activity molecular chaperone is increased or generated in a non-human organism, like a microorganism or a plant cell, plant or part thereof, especially with non-targeted localization. For example, an increase of the linolenic acid of at least 1 percent, particularly in a range of 13 to 24-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a ATPase epsilon subunit, or if the activity of the polypeptide Ypl271w, preferably represented by SEQ ID NO. 118213, or a homolog or fragment thereof, or if the activity of a polypeptide encoded by a nucleic acid molecule comprising the nucleic acid SEQ ID NO. 118212, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 118212 or polypeptide SEQ ID NO. 118213, respectively, or a homolog or a fragment thereof, is increased or generated, or if the activity ATPase epsilon subunit is increased or generated in a non-human organism, like a microorganism or a plant cell, plant or part thereof, especially with plastidic localization. For example, an increase of the linolenic acid of at least 1 percent, particularly in a range of 13 to 27-percent is conferred as compared to a corresponding non-transformed wild type non-human organism.

Accordingly, in one embodiment, an increase of linolenic acid in a non-human organism, as compared to a corresponding non-transformed wild type non-human organism, is conferred in the process of the invention, if the activity of a polypeptide showing the activity of a glucose-6-phosphate 1-dehydrogenase, or if the activity of the polypeptide 59582753_SOYBEAN, preferably represented by SEQ ID NO. 16884, or a homolog or fragment thereof, or if the activity of a polypeptide encoded by a nucleic acid molecule comprising the nucleic acid SEQ ID NO. 16883, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 9, column 5 or 8 in the respective same line as the nucleic acid molecule SEQ ID NO. 16883 or polypeptide SEQ ID NO. 16884, respectively, or a homolog or a fragment thereof, is increased or generated, or if the activity glucose-6-phosphate 1-dehydrogenase is increased or generated in a non-human organism, like a microorganism or a plant cell, plant or part thereof, especially with plastidic localization. For example, an increase of the linolenic acid of at least 1 percent, particularly in a range of 12 to 54-percent is conferred as compared to a corresponding non-transformed wild type non-human organism.

[0096.1.8.9] to [0103.1.8.9] for the disclosure of these paragraphs see [0096.1.8.8] to [0103.1.8.8] above.

In this paragraph “A” means “Goffeau et al., Science 274 (5287), 546 (1996)”, “B” means “Blattner et al., Science 277 (5331), 1453 (1997)”, “C” means “Nakamura et al., Nucleic Acids Res. 28(1), 72 (2000)”, “D” means “deposition of the sequences to the EMBL/GenBank/DDBJ databases under “Sequencing of the draft genome assembly of Azotobacter vinelandii AvOP”. in June 2005 by Copeland et al.”, “E” means “Henne et al., Nat. Biotechnol. 22 (5), 547 (2004)”, “F” means “Kaul et al., Nature 408 (6814), 796 (2000)”, and “G” means “Matsumoto et al., Nature 436 (7052), 793 (2005)”.

The nucleic acid sequence of At1g07430 from Arabidopsis thaliana, e.g. as shown in the respective line in column 5 of Table I, application no. 9, has been published in F. And the activity of the gene product thereof is the activity of protein phosphatase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said At1g07430, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said At1g07430, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said At1g07430, or a functional equivalent or a homolog thereof as depicted in column 8 of Table II, application no. 9, preferably a homolog or functional equivalent as depicted in column 8 of Table II B, application no. 9, and being depicted in the same respective line as said At1g07430, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phos-phatase”, preferably being encoded by a gene comprising the nucleic acid sequence SEQ ID NO. 17451, preferably the coding region thereof, conferred the production of or the increase in linolenic acid compared with the wild type control.

The nucleic acid sequence of At1g68320 from Arabidopsis thaliana, e.g. as shown in the respective line in column 5 of Table I, application no. 9, has been published in F. And the activity of the gene product thereof is the activity of transcription factor.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said At1g68320, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said At1g68320, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said At1g68320, or a functional equivalent or a homolog thereof as depicted in column 8 of Table II, application no. 9, preferably a homolog or functional equivalent as depicted in column 8 of Table II B, application no. 9, and being depicted in the same respective line as said At1g68320, and preferably the activity is increased non-targeted.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 1061, preferably the coding region thereof, conferred the production of or the increase in linolenic acid compared with the wild type control.

The nucleic acid sequence of At1g72770 from Arabidopsis thaliana, e.g. as shown in the respective line in column 5 of Table I, application no. 9, has been published in F. And the activity of the gene product thereof is the activity of protein phosphatase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said At1g72770, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said At1g72770, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said At1g72770, or a functional equivalent or a homolog thereof as depicted in column 8 of Table II, application no. 9, preferably a homolog or functional equivalent as depicted in column 8 of Table II B, application no. 9, and being depicted in the same respective line as said At1g72770, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19419, preferably the coding region thereof, conferred the production of or the increase in linolenic acid compared with the wild type control.

The nucleic acid sequence of At2g36580 from Arabidopsis thaliana, e.g. as shown in the respective line in column 5 of Table I, application no. 9, has been published in F. And the activity of the gene product thereof is the activity of pyruvate kinase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “pyruvate kinase”, especially from Arabidopsis thaliana or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said At2g36580, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said At2g36580, and preferably the activity is increased plastidic, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said At2g36580, or a functional equivalent or a homolog thereof as depicted in column 8 of

Table II, application no. 9, preferably a homolog or functional equivalent as depicted in column 8 of Table II B, application no. 9, and being depicted in the same respective line as said At2g36580, and preferably the activity is increased plastidic.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 113714, preferably the coding region thereof, conferred the production of or the increase in linolenic acid compared with the wild type control.

The nucleic acid sequence of At2g45420 from Arabidopsis thaliana, e.g. as shown in the respective line in column 5 of Table I, application no. 9, has been published in F. And the activity of the gene product thereof is the activity of At2g45420-protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “At2g45420-protein”, especially from Arabidopsis thaliana or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said At2g45420, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said At2g45420, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said At2g45420, or a functional equivalent or a homolog thereof as depicted in column 8 of Table II, application no. 9, preferably a homolog or functional equivalent as depicted in column 8 of Table II B, application no. 9, and being depicted in the same respective line as said At2g45420, and preferably the activity is increased non-targeted.

Accordingly, in one embodiment, the molecule which activity is to be increased in the process of the invention is the gene product with an activity as a “At2g45420-protein”, preferably it is the molecule of section (a) or (b) of this paragraph.

In particular, it was observed that in plants, especially in Arabidopsis thaliana, increasing or generating the activity of a gene product non-targeted with the activity of a “At2g45420-protein”, preferably being encoded by a gene comprising the nucleic acid sequence SEQ ID NO. 1623, preferably the coding region thereof, conferred the production of or the increase in linolenic acid compared with the wild type control.

The nucleic acid sequence of At3g02990 from Arabidopsis thaliana, e.g. as shown in the respective line in column 5 of Table I, application no. 9, has been published in F. And the activity of the gene product thereof is the activity of heat shock transcription factor.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said At3g02990, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said At3g02990, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said At3g02990, or a functional equivalent or a homolog thereof as depicted in column 8 of Table II, application no. 9, preferably a homolog or functional equivalent as depicted in column 8 of Table II B, application no. 9, and being depicted in the same respective line as said At3g02990, and preferably the activity is increased non-targeted.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 68657, preferably the coding region thereof, conferred the production of or the increase in linolenic acid compared with the wild type control.

The nucleic acid sequence of At3g62950 from Arabidopsis thaliana, e.g. as shown in the respective line in column 5 of Table I, application no. 9, has been published in F. And the activity of the gene product thereof is the activity of glutaredoxin.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said At3g62950, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said At3g62950, or a functional equivalent or a homolog thereof as depicted in column 8 of Table II, application no. 9, preferably a homolog or functional equivalent as depicted in column 8 of Table II B, application no. 9, and being depicted in the same respective line as said At3g62950, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in linolenic acid compared with the wild type control.

The nucleic acid sequence of At4g15670 from Arabidopsis thaliana, e.g. as shown in the respective line in column 5 of Table I, application no. 9, has been published in F. And the activity of the gene product thereof is the activity of monothiol glutaredoxin.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said At4g15670, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said At4g15670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said At4g15670, or a functional equivalent or a homolog thereof as depicted in column 8 of

Table II, application no. 9, preferably a homolog or functional equivalent as depicted in column 8 of Table II B, application no. 9, and being depicted in the same respective line as said At4g15670, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutare-doxin”, preferably being encoded by a gene comprising the nucleic acid sequence SEQ ID NO. 3279, preferably the coding region thereof, conferred the production of or the increase in linolenic acid compared with the wild type control.

The nucleic acid sequence of At4g18880 from Arabidopsis thaliana, e.g. as shown in the respective line in column 5 of Table I, application no. 9, has been published in F. And the activity of the gene product thereof is the activity of heat shock transcription factor.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said At4g18880, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said At4g18880, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said At4g18880, or a functional equivalent or a homolog thereof as depicted in column 8 of Table II, application no. 9, preferably a homolog or functional equivalent as depicted in column 8 of Table II B, application no. 9, and being depicted in the same respective line as said At4g18880, and preferably the activity is increased non-targeted.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 24232, preferably the coding region thereof, conferred the production of or the increase in linolenic acid compared with the wild type control.

The nucleic acid sequence of At4g26080 from Arabidopsis thaliana, e.g. as shown in the respective line in column 5 of Table I, application no. 9, has been published in F. And the activity of the gene product thereof is the activity of protein phosphatase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said At4g26080, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said At4g26080, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said At4g26080, or a functional equivalent or a homolog thereof as depicted in column 8 of Table II, application no. 9, preferably a homolog or functional equivalent as depicted in column 8 of Table II B, application no. 9, and being depicted in the same respective line as said At4g26080, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 68849, preferably the coding region thereof, conferred the production of or the increase in linolenic acid compared with the wild type control.

The nucleic acid sequence of At4g33040 from Arabidopsis thaliana, e.g. as shown in the respective line in column 5 of Table I, application no. 9, has been published in F. And the activity of the gene product thereof is the activity of glutaredoxin.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said At4g33040, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said At4g33040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said At4g33040, or a functional equivalent or a homolog thereof as depicted in column 8 of Table II, application no. 9, preferably a homolog or functional equivalent as depicted in column 8 of Table II B, application no. 9, and being depicted in the same respective line as said At4g33040, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 4102, preferably the coding region thereof, conferred the production of or the increase in linolenic acid compared with the wild type control.

The nucleic acid sequence of At5g03720 from Arabidopsis thaliana, e.g. as shown in the respective line in column 5 of Table I, application no. 9, has been published in F. And the activity of the gene product thereof is the activity of heat shock transcription factor.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said At5g03720, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said At5g03720, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said At5g03720, or a functional equivalent or a homolog thereof as depicted in column 8 of

Table II, application no. 9, preferably a homolog or functional equivalent as depicted in column 8 of Table II B, application no. 9, and being depicted in the same respective line as said At5g03720, and preferably the activity is increased non-targeted.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 24438, preferably the coding region thereof, conferred the production of or the increase in linolenic acid compared with the wild type control.

The nucleic acid sequence of At5g18600 from Arabidopsis thaliana, e.g. as shown in the respective line in column 5 of Table I, application no. 9, has been published in F. And the activity of the gene product thereof is the activity of monothiol glutaredoxin.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said At5g18600, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said At5g18600, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said At5g18600, or a functional equivalent or a homolog thereof as depicted in column 8 of Table II, application no. 9, preferably a homolog or functional equivalent as depicted in column 8 of Table II B, application no. 9, and being depicted in the same respective line as said At5g18600, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutare-doxin”, preferably being encoded by a gene comprising the nucleic acid sequence SEQ ID NO. 4904, preferably the coding region thereof, conferred the production of or the increase in linolenic acid compared with the wild type control.

The nucleic acid sequence of At5g57050 from Arabidopsis thaliana, e.g. as shown in the respective line in column 5 of Table I, application no. 9, has been published in F. And the activity of the gene product thereof is the activity of protein phosphatase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said At5g57050, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said At5g57050, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said At5g57050, or a functional equivalent or a homolog thereof as depicted in column 8 of Table II, application no. 9, preferably a homolog or functional equivalent as depicted in column 8 of Table II B, application no. 9, and being depicted in the same respective line as said At5g57050, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 5318, preferably the coding region thereof, conferred the production of or the increase in linolenic acid compared with the wild type control.

The nucleic acid sequence of At5g59220 from Arabidopsis thaliana, e.g. as shown in the respective line in column 5 of Table I, application no. 9, has been published in F. And the activity of the gene product thereof is the activity of protein phosphatase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said At5g59220, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said At5g59220, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said At5g59220, or a functional equivalent or a homolog thereof as depicted in column 8 of Table II, application no. 9, preferably a homolog or functional equivalent as depicted in column 8 of Table II B, application no. 9, and being depicted in the same respective line as said At5g59220, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phos-phatase”, preferably being encoded by a gene comprising the nucleic acid sequence SEQ ID NO. 25498, preferably the coding region thereof, conferred the production of or the increase in linolenic acid compared with the wild type control.

The nucleic acid sequence of AvinDRAFT_{—}1624 from Azotobacter vinelandii, e.g. as shown in the respective line in column 5 of Table I, application no. 9, has been published in D. And the activity of the gene product thereof is the activity of sec-independent protein translocase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “sec-independent protein translocase”, especially from Azotobacter vinelandii or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said AvinDRAFT_{—}1624, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said AvinDRAFT_{—}1624, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said AvinDRAFT_{—}1624, or a functional equivalent or a homolog thereof as depicted in column 8 of Table II, application no. 9, preferably a homolog or functional equivalent as depicted in column 8 of Table II B, application no. 9, and being depicted in the same respective line as said AvinDRAFT_{—}1624, and preferably the activity is increased non-targeted.

Accordingly, in one embodiment, the molecule which activity is to be increased in the process of the invention is the gene product with an activity as a “sec-independent protein translocase”, preferably it is the molecule of section (a) or (b) of this paragraph.

In particular, it was observed that in plants, especially in Arabidopsis thaliana, increasing or generating the activity of a gene product non-targeted with the activity of a “sec-independent protein translocase”, preferably being encoded by a gene comprising the nucleic acid sequence SEQ ID NO. 26196, preferably the coding region thereof, conferred the production of or the increase in linolenic acid compared with the wild type control.

The nucleic acid sequence of AvinDRAFT_{—}1788 from Azotobacter vinelandii, e.g. as shown in the respective line in column 5 of Table I, application no. 9, has been published in D. And the activity of the gene product thereof is the activity of oxidoreductase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “oxidoreductase”, especially from Azotobacter vinelandii or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said AvinDRAFT_{—}1788, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said AvinDRAFT_{—}1788, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said AvinDRAFT_{—}1788, or a functional equivalent or a homolog thereof as depicted in column 8 of Table II, application no. 9, preferably a homolog or functional equivalent as depicted in column 8 of Table II B, application no. 9, and being depicted in the same respective line as said AvinDRAFT_{—}1788, and preferably the activity is increased non-targeted.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 114083, preferably the coding region thereof, conferred the production of or the increase in linolenic acid compared with the wild type control.

The nucleic acid sequence of AvinDRAFT_{—}2754 from Azotobacter vinelandii, e.g. as shown in the respective line in column 5 of Table I, application no. 9, has been published in D. And the activity of the gene product thereof is the activity of acyl-CoA synthase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “acyl-CoA synthase”, especially from Azotobacter vinelandii or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said AvinDRAFT_{—}2754, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said AvinDRAFT_{—}2754, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said AvinDRAFT_{—}2754, or a functional equivalent or a homolog thereof as depicted in column 8 of Table II, application no. 9, preferably a homolog or functional equivalent as depicted in column 8 of Table II B, application no. 9, and being depicted in the same respective line as said AvinDRAFT_{—}2754, and preferably the activity is increased non-targeted.

Accordingly, in one embodiment, the molecule which activity is to be increased in the process of the invention is the gene product with an activity as a “acyl-CoA synthase”, preferably it is the molecule of section (a) or (b) of this paragraph.

In particular, it was observed that in plants, especially in Arabidopsis thaliana, increasing or generating the activity of a gene product non-targeted with the activity of a “acyl-CoA synthase”, preferably being encoded by a gene comprising the nucleic acid sequence SEQ ID NO. 28040, preferably the coding region thereof, conferred the production of or the increase in linolenic acid compared with the wild type control.

The nucleic acid sequence of AvinDRAFT_{—}3253 from Azotobacter vinelandii, e.g. as shown in the respective line in column 5 of Table I, application no. 9, has been published in D. And the activity of the gene product thereof is the activity of oxidoreductase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “oxidoreductase”, especially from Azotobacter vinelandii or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said AvinDRAFT_{—}3253, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said AvinDRAFT_{—}3253, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said AvinDRAFT_{—}3253, or a functional equivalent or a homolog thereof as depicted in column 8 of Table II, application no. 9, preferably a homolog or functional equivalent as depicted in column 8 of Table II B, application no. 9, and being depicted in the same respective line as said AvinDRAFT_{—}3253, and preferably the activity is increased non-targeted.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 29500, preferably the coding region thereof, conferred the production of or the increase in linolenic acid compared with the wild type control.

The nucleic acid sequence of AvinDRAFT_{—}3577 from Azotobacter vinelandii, e.g. as shown in the respective line in column 5 of Table I, application no. 9, has been published in D. And the activity of the gene product thereof is the activity of ThiF family protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “ThiF family protein”, especially from Azotobacter vinelandii or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said AvinDRAFT_{—}3577, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said AvinDRAFT_{—}3577, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said AvinDRAFT_{—}3577, or a functional equivalent or a homolog thereof as depicted in column 8 of Table II, application no. 9, preferably a homolog or functional equivalent as depicted in column 8 of Table II B, application no. 9, and being depicted in the same respective line as said AvinDRAFT_{—}3577, and preferably the activity is increased non-targeted.

Accordingly, in one embodiment, the molecule which activity is to be increased in the process of the invention is the gene product with an activity as a “ThiF family protein”, preferably it is the molecule of section (a) or (b) of this paragraph.

In particular, it was observed that in plants, especially in Arabidopsis thaliana, increasing or generating the activity of a gene product non-targeted with the activity of a “ThiF family protein”, preferably being encoded by a gene comprising the nucleic acid sequence SEQ ID NO. 111155, preferably the coding region thereof, conferred the production of or the increase in linolenic acid compared with the wild type control.

The nucleic acid sequence of AvinDRAFT_{—}3605 from Azotobacter vinelandii, e.g. as shown in the respective line in column 5 of Table I, application no. 9, has been published in D. And the activity of the gene product thereof is the activity of CTP synthase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “CTP synthase”, especially from Azotobacter vinelandii or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said AvinDRAFT_{—}3605, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said AvinDRAFT_{—}3605, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said AvinDRAFT_{—}3605, or a functional equivalent or a homolog thereof as depicted in column 8 of Table II, application no. 9, preferably a homolog or functional equivalent as depicted in column 8 of Table II B, application no. 9, and being depicted in the same respective line as said AvinDRAFT_{—}3605, and preferably the activity is increased non-targeted.

Accordingly, in one embodiment, the molecule which activity is to be increased in the process of the invention is the gene product with an activity as a “CTP synthase”, preferably it is the molecule of section (a) or (b) of this paragraph.

In particular, it was observed that in plants, especially in Arabidopsis thaliana, increasing or generating the activity of a gene product non-targeted with the activity of a “CTP synthase”, preferably being encoded by a gene comprising the nucleic acid sequence SEQ ID NO. 31026, preferably the coding region thereof, conferred the production of or the increase in linolenic acid compared with the wild type control.

The nucleic acid sequence of AvinDRAFT_{—}3629 from Azotobacter vinelandii, e.g. as shown in the respective line in column 5 of Table I, application no. 9, has been published in D. And the activity of the gene product thereof is the activity of recombinase A.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “recombinase A”, especially from Azotobacter vinelandii or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said AvinDRAFT_{—}3629, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said AvinDRAFT_{—}3629, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said AvinDRAFT_{—}3629, or a functional equivalent or a homolog thereof as depicted in column 8 of Table II, application no. 9, preferably a homolog or functional equivalent as depicted in column 8 of Table II B, application no. 9, and being depicted in the same respective line as said AvinDRAFT_{—}3629, and preferably the activity is increased non-targeted.

Accordingly, in one embodiment, the molecule which activity is to be increased in the process of the invention is the gene product with an activity as a “recombinase A”, preferably it is the molecule of section (a) or (b) of this paragraph.

In particular, it was observed that in plants, especially in Arabidopsis thaliana, increasing or generating the activity of a gene product non-targeted with the activity of a “recombinase A”, preferably being encoded by a gene comprising the nucleic acid sequence SEQ ID NO. 114231, preferably the coding region thereof, conferred the production of or the increase in linolenic acid compared with the wild type control.

The nucleic acid sequence of AvinDRAFT_{—}4847 from Azotobacter vinelandii, e.g. as shown in the respective line in column 5 of Table I, application no. 9, has been published in D. And the activity of the gene product thereof is the activity of isopropylmalate isomerase large subunit.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “isopropylmalate isomerase large subunit”, especially from Azotobacter vinelandii or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said AvinDRAFT_{—}4847, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said AvinDRAFT_{—}4847, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said AvinDRAFT_{—}4847, or a functional equivalent or a homolog thereof as depicted in column 8 of Table II, application no. 9, preferably a homolog or functional equivalent as depicted in column 8 of Table II B, application no. 9, and being depicted in the same respective line as said AvinDRAFT_{—}4847, and preferably the activity is increased non-targeted.

Accordingly, in one embodiment, the molecule which activity is to be increased in the process of the invention is the gene product with an activity as a “isopropylmalate isomerase large sub-unit”, preferably it is the molecule of section (a) or (b) of this paragraph.

In particular, it was observed that in plants, especially in Arabidopsis thaliana, increasing or generating the activity of a gene product non-targeted with the activity of a “isopropylmalate isomerase large subunit”, preferably being encoded by a gene comprising the nucleic acid sequence SEQ ID NO. 102941, preferably the coding region thereof, conferred the production of or the increase in linolenic acid compared with the wild type control.

The nucleic acid sequence of AvinDRAFT_{—}5467 from Azotobacter vinelandii, e.g. as shown in the respective line in column 5 of Table I, application no. 9, has been published in D. And the activity of the gene product thereof is the activity of HesB/YadR/YfhF family protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “HesB/YadR/YfhF family protein”, especially from Azotobacter vinelandii or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said AvinDRAFT_{—}5467, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said AvinDRAFT_{—}5467, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said AvinDRAFT_{—}5467, or a functional equivalent or a homolog thereof as depicted in column 8 of Table II, application no. 9, preferably a homolog or functional equivalent as depicted in column 8 of Table II B, application no. 9, and being depicted in the same respective line as said AvinDRAFT_{—}5467, and preferably the activity is increased non-targeted.

Accordingly, in one embodiment, the molecule which activity is to be increased in the process of the invention is the gene product with an activity as a “HesB/YadR/YfhF family protein”, preferably it is the molecule of section (a) or (b) of this paragraph.

In particular, it was observed that in plants, especially in Arabidopsis thaliana, increasing or generating the activity of a gene product non-targeted with the activity of a “HesB/YadR/YfhF family protein”, preferably being encoded by a gene comprising the nucleic acid sequence SEQ ID NO. 32648, preferably the coding region thereof, conferred the production of or the increase in linolenic acid compared with the wild type control.

The nucleic acid sequence of B0195 from Escherichia coli, e.g. as shown in the respective line in column 5 of Table I, application no. 9, has been published in B. And the activity of the gene product thereof is the activity of b0195-protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “b0195-protein”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B0195, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B0195, and preferably the activity is increased plastidic, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B0195, or a functional equivalent or a homolog thereof as depicted in column 8 of Table II, application no. 9, preferably a homolog or functional equivalent as depicted in column 8 of Table II B, application no. 9, and being depicted in the same respective line as said B0195, and preferably the activity is increased plastidic.

Accordingly, in one embodiment, the molecule which activity is to be increased in the process of the invention is the gene product with an activity as a “b0195-protein”, preferably it is the molecule of section (a) or (b) of this paragraph.

In particular, it was observed that in plants, especially in Arabidopsis thaliana, increasing or generating the activity of a gene product plastidic with the activity of a “b0195-protein”, preferably being encoded by a gene comprising the nucleic acid sequence SEQ ID NO. 118238, preferably the coding region thereof, conferred the production of or the increase in linolenic acid compared with the wild type control.

The nucleic acid sequence of B0828 from Escherichia coli, e.g. as shown in the respective line in column 5 of Table I, application no. 9, has been published in B. And the activity of the gene product thereof is the activity of asparaginase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “asparaginase”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B0828, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B0828, and preferably the activity is increased plastidic, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B0828, or a functional equivalent or a homolog thereof as depicted in column 8 of Table II, application no. 9, preferably a homolog or functional equivalent as depicted in column 8 of Table II B, application no. 9, and being depicted in the same respective line as said B0828, and preferably the activity is increased plastidic.

Accordingly, in one embodiment, the molecule which activity is to be increased in the process of the invention is the gene product with an activity as a “asparaginase”, preferably it is the molecule of section (a) or (b) of this paragraph.

In particular, it was observed that in plants, especially in Arabidopsis thaliana, increasing or generating the activity of a gene product plastidic with the activity of a “asparaginase”, preferably being encoded by a gene comprising the nucleic acid sequence SEQ ID NO. 36299, preferably the coding region thereof, conferred the production of or the increase in linolenic acid compared with the wild type control.

The nucleic acid sequence of B1065 from Escherichia coli, e.g. as shown in the respective line in column 5 of Table I, application no. 9, has been published in B. And the activity of the gene product thereof is the activity of multidrug resistance protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “multidrug resistance protein”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B1065, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B1065, and preferably the activity is increased plastidic, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B1065, or a functional equivalent or a homolog thereof as depicted in column 8 of Table II, application no. 9, preferably a homolog or functional equivalent as depicted in column 8 of Table II B, application no. 9, and being depicted in the same respective line as said B1065, and preferably the activity is increased plastidic.

Accordingly, in one embodiment, the molecule which activity is to be increased in the process of the invention is the gene product with an activity as a “multidrug resistance protein”, preferably it is the molecule of section (a) or (b) of this paragraph.

In particular, it was observed that in plants, especially in Arabidopsis thaliana, increasing or generating the activity of a gene product plastidic with the activity of a “multidrug resistance protein”, preferably being encoded by a gene comprising the nucleic acid sequence SEQ ID NO. 76032, preferably the coding region thereof, conferred the production of or the increase in linolenic acid compared with the wild type control.

The nucleic acid sequence of B1093 from Escherichia coli, e.g. as shown in the respective line in column 5 of Table I, application no. 9, has been published in B. And the activity of the gene product thereof is the activity of 3-oxoacyl-[acyl-carrier-protein] reductase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “3-oxoacyl-[acyl-carrier-protein] reductase”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B1093, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B1093, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B1093, or a functional equivalent or a homolog thereof as depicted in column 8 of Table II, application no. 9, preferably a homolog or functional equivalent as depicted in column 8 of Table II B, application no. 9, and being depicted in the same respective line as said B1093, and preferably the activity is increased non-targeted.

Accordingly, in one embodiment, the molecule which activity is to be increased in the process of the invention is the gene product with an activity as a “3-oxoacyl-[acyl-carrier-protein] reductase”, preferably it is the molecule of section (a) or (b) of this paragraph.

In particular, it was observed that in plants, especially in Arabidopsis thaliana, increasing or generating the activity of a gene product non-targeted with the activity of a “3-oxoacyl-[acyl-carrier-protein] reductase”, preferably being encoded by a gene comprising the nucleic acid sequence SEQ ID NO. 118371, preferably the coding region thereof, conferred the production of or the increase in linolenic acid compared with the wild type control.

The nucleic acid sequence of B1095 from Escherichia coli, e.g. as shown in the respective line in column 5 of Table I, application no. 9, has been published in B. And the activity of the gene product thereof is the activity of 3-oxoacyl-[acyl-carrier-protein] synthase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “3-oxoacyl-[acyl-carrier-protein] synthase”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B1095, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B1095, and preferably the activity is increased plastidic, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B1095, or a functional equivalent or a homolog thereof as depicted in column 8 of Table II, application no. 9, preferably a homolog or functional equivalent as depicted in column 8 of Table II B, application no. 9, and being depicted in the same respective line as said B1095, and preferably the activity is increased plastidic.

Accordingly, in one embodiment, the molecule which activity is to be increased in the process of the invention is the gene product with an activity as a “3-oxoacyl-[acyl-carrier-protein] synthase”, preferably it is the molecule of section (a) or (b) of this paragraph.

In particular, it was observed that in plants, especially in Arabidopsis thaliana, increasing or generating the activity of a gene product plastidic with the activity of a “3-oxoacyl-[acyl-carrier-protein] synthase”, preferably being encoded by a gene comprising the nucleic acid sequence SEQ ID NO. 114552, preferably the coding region thereof, conferred the production of or the increase in linolenic acid compared with the wild type control.

The nucleic acid sequence of B1234 from Escherichia coli, e.g. as shown in the respective line in column 5 of Table I, application no. 9, has been published in B. And the activity of the gene product thereof is the activity of b1234-protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “b1234-protein”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B1234, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B1234, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B1234, or a functional equivalent or a homolog thereof as depicted in column 8 of Table II, application no. 9, preferably a homolog or functional equivalent as depicted in column 8 of Table II B, application no. 9, and being depicted in the same respective line as said B1234, and preferably the activity is increased non-targeted.

Accordingly, in one embodiment, the molecule which activity is to be increased in the process of the invention is the gene product with an activity as a “b1234-protein”, preferably it is the molecule of section (a) or (b) of this paragraph.

In particular, it was observed that in plants, especially in Arabidopsis thaliana, increasing or generating the activity of a gene product non-targeted with the activity of a “b1234-protein”, preferably being encoded by a gene comprising the nucleic acid sequence SEQ ID NO. 112111, preferably the coding region thereof, conferred the production of or the increase in linolenic acid compared with the wild type control.

The nucleic acid sequence of B1248 from Escherichia coli, e.g. as shown in the respective line in column 5 of Table I, application no. 9, has been published in B. And the activity of the gene product thereof is the activity of b1248-protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “b1248-protein”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B1248, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B1248, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B1248, or a functional equivalent or a homolog thereof as depicted in column 8 of Table II, application no. 9, preferably a homolog or functional equivalent as depicted in column 8 of Table II B, application no. 9, and being depicted in the same respective line as said B1248, and preferably the activity is increased non-targeted.

Accordingly, in one embodiment, the molecule which activity is to be increased in the process of the invention is the gene product with an activity as a “b1248-protein”, preferably it is the molecule of section (a) or (b) of this paragraph.

In particular, it was observed that in plants, especially in Arabidopsis thaliana, increasing or generating the activity of a gene product non-targeted with the activity of a “b1248-protein”, preferably being encoded by a gene comprising the nucleic acid sequence SEQ ID NO. 119135, preferably the coding region thereof, conferred the production of or the increase in linolenic acid compared with the wild type control.

The nucleic acid sequence of B1280 from Escherichia coli, e.g. as shown in the respective line in column 5 of Table I, application no. 9, has been published in B. And the activity of the gene product thereof is the activity of b1280-protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “b1280-protein”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B1280, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B1280, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B1280, or a functional equivalent or a homolog thereof as depicted in column 8 of Table II, application no. 9, preferably a homolog or functional equivalent as depicted in column 8 of Table II B, application no. 9, and being depicted in the same respective line as said B1280, and preferably the activity is increased non-targeted.

Accordingly, in one embodiment, the molecule which activity is to be increased in the process of the invention is the gene product with an activity as a “b1280-protein”, preferably it is the molecule of section (a) or (b) of this paragraph.

In particular, it was observed that in plants, especially in Arabidopsis thaliana, increasing or generating the activity of a gene product non-targeted with the activity of a “b1280-protein”, preferably being encoded by a gene comprising the nucleic acid sequence SEQ ID NO. 37573, preferably the coding region thereof, conferred the production of or the increase in linolenic acid compared with the wild type control.

The nucleic acid sequence of B1330 from Escherichia coli, e.g. as shown in the respective line in column 5 of Table I, application no. 9, has been published in B. And the activity of the gene product thereof is the activity of b1330-protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “b1330-protein”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B1330, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B1330, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B1330, or a functional equivalent or a homolog thereof as depicted in column 8 of Table II, application no. 9, preferably a homolog or functional equivalent as depicted in column 8 of Table II B, application no. 9, and being depicted in the same respective line as said B1330, and preferably the activity is increased non-targeted.

Accordingly, in one embodiment, the molecule which activity is to be increased in the process of the invention is the gene product with an activity as a “b1330-protein”, preferably it is the molecule of section (a) or (b) of this paragraph.

In particular, it was observed that in plants, especially in Arabidopsis thaliana, increasing or generating the activity of a gene product non-targeted with the activity of a “b1330-protein”, preferably being encoded by a gene comprising the nucleic acid sequence SEQ ID NO. 38226, preferably the coding region thereof, conferred the production of or the increase in linolenic acid compared with the wild type control.

The nucleic acid sequence of B1470 from Escherichia coli, e.g. as shown in the respective line in column 5 of Table I, application no. 9, has been published in B. And the activity of the gene product thereof is the activity of b1470-protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “b1470-protein”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B1470, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B1470, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B1470, or a functional equivalent or a homolog thereof as depicted in column 8 of Table II, application no. 9, preferably a homolog or functional equivalent as depicted in column 8 of Table II B, application no. 9, and being depicted in the same respective line as said B1470, and preferably the activity is increased non-targeted.

Accordingly, in one embodiment, the molecule which activity is to be increased in the process of the invention is the gene product with an activity as a “b1470-protein”, preferably it is the molecule of section (a) or (b) of this paragraph.

In particular, it was observed that in plants, especially in Arabidopsis thaliana, increasing or generating the activity of a gene product non-targeted with the activity of a “b1470-protein”, preferably being encoded by a gene comprising the nucleic acid sequence SEQ ID NO. 115056, preferably the coding region thereof, conferred the production of or the increase in linolenic acid compared with the wild type control.

The nucleic acid sequence of B1597 from Escherichia coli, e.g. as shown in the respective line in column 5 of Table I, application no. 9, has been published in B. And the activity of the gene product thereof is the activity of acid shock protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “acid shock protein”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B1597, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B1597, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B1597, or a functional equivalent or a homolog thereof as depicted in column 8 of Table II, application no. 9, preferably a homolog or functional equivalent as depicted in column 8 of Table II B, application no. 9, and being depicted in the same respective line as said B1597, and preferably the activity is increased non-targeted.

Accordingly, in one embodiment, the molecule which activity is to be increased in the process of the invention is the gene product with an activity as a “acid shock protein”, preferably it is the molecule of section (a) or (b) of this paragraph.

In particular, it was observed that in plants, especially in Arabidopsis thaliana, increasing or generating the activity of a gene product non-targeted with the activity of a “acid shock protein”, preferably being encoded by a gene comprising the nucleic acid sequence SEQ ID NO. 38300, preferably the coding region thereof, conferred the production of or the increase in linolenic acid compared with the wild type control.

The nucleic acid sequence of B1611 from Escherichia coli, e.g. as shown in the respective line in column 5 of Table I, application no. 9, has been published in B. And the activity of the gene product thereof is the activity of fumarate hydratase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “fumarate hydratase”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B1611, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B1611, and preferably the activity is increased plastidic, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B1611, or a functional equivalent or a homolog thereof as depicted in column 8 of Table II, application no. 9, preferably a homolog or functional equivalent as depicted in column 8 of Table II B, application no. 9, and being depicted in the same respective line as said B1611, and preferably the activity is increased plastidic.

Accordingly, in one embodiment, the molecule which activity is to be increased in the process of the invention is the gene product with an activity as a “fumarate hydratase”, preferably it is the molecule of section (a) or (b) of this paragraph.

In particular, it was observed that in plants, especially in Arabidopsis thaliana, increasing or generating the activity of a gene product plastidic with the activity of a “fumarate hydratase”, preferably being encoded by a gene comprising the nucleic acid sequence SEQ ID NO. 115064, preferably the coding region thereof, conferred the production of or the increase in linolenic acid compared with the wild type control.

The nucleic acid sequence of B1627 from Escherichia coli, e.g. as shown in the respective line in column 5 of Table I, application no. 9, has been published in B. And the activity of the gene product thereof is the activity of electron transport complex protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “electron transport complex protein”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B1627, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B1627, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B1627, or a functional equivalent or a homolog thereof as depicted in column 8 of Table II, application no. 9, preferably a homolog or functional equivalent as depicted in column 8 of Table II B, application no. 9, and being depicted in the same respective line as said B1627, and preferably the activity is increased non-targeted.

Accordingly, in one embodiment, the molecule which activity is to be increased in the process of the invention is the gene product with an activity as a “electron transport complex protein”, preferably it is the molecule of section (a) or (b) of this paragraph.

In particular, it was observed that in plants, especially in Arabidopsis thaliana, increasing or generating the activity of a gene product non-targeted with the activity of a “electron transport complex protein”, preferably being encoded by a gene comprising the nucleic acid sequence SEQ ID NO. 38345, preferably the coding region thereof, conferred the production of or the increase in linolenic acid compared with the wild type control.

The nucleic acid sequence of B1670 from Escherichia coli, e.g. as shown in the respective line in column 5 of Table I, application no. 9, has been published in B. And the activity of the gene product thereof is the activity of b1670-protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “b1670-protein”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B1670, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B1670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B1670, or a functional equivalent or a homolog thereof as depicted in column 8 of Table II, application no. 9, preferably a homolog or functional equivalent as depicted in column 8 of Table II B, application no. 9, and being depicted in the same respective line as said B1670, and preferably the activity is increased non-targeted.

Accordingly, in one embodiment, the molecule which activity is to be increased in the process of the invention is the gene product with an activity as a “b1670-protein”, preferably it is the molecule of section (a) or (b) of this paragraph.

In particular, it was observed that in plants, especially in Arabidopsis thaliana, increasing or generating the activity of a gene product non-targeted with the activity of a “b1670-protein”, preferably being encoded by a gene comprising the nucleic acid sequence SEQ ID NO. 78753, preferably the coding region thereof, conferred the production of or the increase in linolenic acid compared with the wild type control.

The nucleic acid sequence of B1672 from Escherichia coli, e.g. as shown in the respective line in column 5 of Table I, application no. 9, has been published in B. And the activity of the gene product thereof is the activity of b1672-protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “b1672-protein”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B1672, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B1672, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B1672, or a functional equivalent or a homolog thereof as depicted in column 8 of Table II, application no. 9, preferably a homolog or functional equivalent as depicted in column 8 of Table II B, application no. 9, and being depicted in the same respective line as said B1672, and preferably the activity is increased non-targeted.

Accordingly, in one embodiment, the molecule which activity is to be increased in the process of the invention is the gene product with an activity as a “b1672-protein”, preferably it is the molecule of section (a) or (b) of this paragraph.

In particular, it was observed that in plants, especially in Arabidopsis thaliana, increasing or generating the activity of a gene product non-targeted with the activity of a “b1672-protein”, preferably being encoded by a gene comprising the nucleic acid sequence SEQ ID NO. 78771, preferably the coding region thereof, conferred the production of or the increase in linolenic acid compared with the wild type control.

The nucleic acid sequence of B1837 from Escherichia coli, e.g. as shown in the respective line in column 5 of Table I, application no. 9, has been published in B. And the activity of the gene product thereof is the activity of b1837-protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “b1837-protein”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B1837, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B1837, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B1837, or a functional equivalent or a homolog thereof as depicted in column 8 of Table II, application no. 9, preferably a homolog or functional equivalent as depicted in column 8 of Table II B, application no. 9, and being depicted in the same respective line as said B1837, and preferably the activity is increased non-targeted.

Accordingly, in one embodiment, the molecule which activity is to be increased in the process of the invention is the gene product with an activity as a “b1837-protein”, preferably it is the molecule of section (a) or (b) of this paragraph.

In particular, it was observed that in plants, especially in Arabidopsis thaliana, increasing or generating the activity of a gene product non-targeted with the activity of a “b1837-protein”, preferably being encoded by a gene comprising the nucleic acid sequence SEQ ID NO. 78953, preferably the coding region thereof, conferred the production of or the increase in linolenic acid compared with the wild type control.

The nucleic acid sequence of B2032 from Escherichia coli, e.g. as shown in the respective line in column 5 of Table I, application no. 9, has been published in B. And the activity of the gene product thereof is the activity of b2032-protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “b2032-protein”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B2032, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B2032, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B2032, or a functional equivalent or a homolog thereof as depicted in column 8 of Table II, application no. 9, preferably a homolog or functional equivalent as depicted in column 8 of Table II B, application no. 9, and being depicted in the same respective line as said B2032, and preferably the activity is increased non-targeted.

Accordingly, in one embodiment, the molecule which activity is to be increased in the process of the invention is the gene product with an activity as a “b2032-protein”, preferably it is the molecule of section (a) or (b) of this paragraph.

In particular, it was observed that in plants, especially in Arabidopsis thaliana, increasing or generating the activity of a gene product non-targeted with the activity of a “b2032-protein”, preferably being encoded by a gene comprising the nucleic acid sequence SEQ ID NO. 8920, preferably the coding region thereof, conferred the production of or the increase in linolenic acid compared with the wild type control.

The nucleic acid sequence of B2107 from Escherichia coli, e.g. as shown in the respective line in column 5 of Table I, application no. 9, has been published in B. And the activity of the gene product thereof is the activity of b2107-protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “b2107-protein”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B2107, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B2107, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B2107, or a functional equivalent or a homolog thereof as depicted in column 8 of Table II, application no. 9, preferably a homolog or functional equivalent as depicted in column 8 of Table II B, application no. 9, and being depicted in the same respective line as said B2107, and preferably the activity is increased non-targeted.

Accordingly, in one embodiment, the molecule which activity is to be increased in the process of the invention is the gene product with an activity as a “b2107-protein”, preferably it is the molecule of section (a) or (b) of this paragraph.

In particular, it was observed that in plants, especially in Arabidopsis thaliana, increasing or generating the activity of a gene product non-targeted with the activity of a “b2107-protein”, preferably being encoded by a gene comprising the nucleic acid sequence SEQ ID NO. 39002, preferably the coding region thereof, conferred the production of or the increase in linolenic acid compared with the wild type control.

The nucleic acid sequence of B2399 from Escherichia coli, e.g. as shown in the respective line in column 5 of Table I, application no. 9, has been published in B. And the activity of the gene product thereof is the activity of b2399-protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “b2399-protein”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B2399, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B2399, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said functional2399, or a functional equivalent or a homolog thereof as depicted in column 8 of Table II, application no. 9, preferably a homolog or functional equivalent as depicted in column 8 of Table II B, application no. 9, and being depicted in the same respective line as said B2399, and preferably the activity is increased non-targeted.

Accordingly, in one embodiment, the molecule which activity is to be increased in the process of the invention is the gene product with an activity as a “b2399-protein”, preferably it is the molecule of section (a) or (b) of this paragraph.

In particular, it was observed that in plants, especially in Arabidopsis thaliana, increasing or generating the activity of a gene product non-targeted with the activity of a “b2399-protein”, preferably being encoded by a gene comprising the nucleic acid sequence SEQ ID NO. 39237, preferably the coding region thereof, conferred the production of or the increase in linolenic acid compared with the wild type control.

The nucleic acid sequence of B2439 from Escherichia coli, e.g. as shown in the respective line in column 5 of Table I, application no. 9, has been published in B. And the activity of the gene product thereof is the activity of ethanolamine utilization protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “ethanolamine utilization protein”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B2439, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B2439, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B2439, or a functional equivalent or a homolog thereof as depicted in column 8 of Table II, application no. 9, preferably a homolog or functional equivalent as depicted in column 8 of Table II B, application no. 9, and being depicted in the same respective line as said B2439, and preferably the activity is increased non-targeted.

Accordingly, in one embodiment, the molecule which activity is to be increased in the process of the invention is the gene product with an activity as a “ethanolamine utilization protein”, preferably it is the molecule of section (a) or (b) of this paragraph.

In particular, it was observed that in plants, especially in Arabidopsis thaliana, increasing or generating the activity of a gene product non-targeted with the activity of a “ethanolamine utilization protein”, preferably being encoded by a gene comprising the nucleic acid sequence SEQ ID NO. 115628, preferably the coding region thereof, conferred the production of or the increase in linolenic acid compared with the wild type control.

The nucleic acid sequence of B2474 from Escherichia coli, e.g. as shown in the respective line in column 5 of Table I, application no. 9, has been published in B. And the activity of the gene product thereof is the activity of b2474-protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “b2474-protein”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B2474, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B2474, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B2474, or a functional equivalent or a homolog thereof as depicted in column 8 of Table II, application no. 9, preferably a homolog or functional equivalent as depicted in column 8 of Table II B, application no. 9, and being depicted in the same respective line as said B2474, and preferably the activity is increased non-targeted.

Accordingly, in one embodiment, the molecule which activity is to be increased in the process of the invention is the gene product with an activity as a “b2474-protein”, preferably it is the molecule of section (a) or (b) of this paragraph.

In particular, it was observed that in plants, especially in Arabidopsis thaliana, increasing or generating the activity of a gene product non-targeted with the activity of a “b2474-protein”, preferably being encoded by a gene comprising the nucleic acid sequence SEQ ID NO. 40329, preferably the coding region thereof, conferred the production of or the increase in linolenic acid compared with the wild type control.

The nucleic acid sequence of B2613 from Escherichia coli, e.g. as shown in the respective line in column 5 of Table I, application no. 9, has been published in B. And the activity of the gene product thereof is the activity of b2613-protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “b2613-protein”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B2613, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B2613, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B2613, or a functional equivalent or a homolog thereof as depicted in column 8 of Table II, application no. 9, preferably a homolog or functional equivalent as depicted in column 8 of Table II B, application no. 9, and being depicted in the same respective line as said B2613, and preferably the activity is increased non-targeted.

Accordingly, in one embodiment, the molecule which activity is to be increased in the process of the invention is the gene product with an activity as a “b2613-protein”, preferably it is the molecule of section (a) or (b) of this paragraph.

In particular, it was observed that in plants, especially in Arabidopsis thaliana, increasing or generating the activity of a gene product non-targeted with the activity of a “b2613-protein”, preferably being encoded by a gene comprising the nucleic acid sequence SEQ ID NO. 40665, preferably the coding region thereof, conferred the production of or the increase in linolenic acid compared with the wild type control.

The nucleic acid sequence of B2682 from Escherichia coli, e.g. as shown in the respective line in column 5 of Table I, application no. 9, has been published in B. And the activity of the gene product thereof is the activity of b2682-protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “b2682-protein”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B2682, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B2682, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B2682, or a functional equivalent or a homolog thereof as depicted in column 8 of Table II, application no. 9, preferably a homolog or functional equivalent as depicted in column 8 of Table II B, application no. 9, and being depicted in the same respective line as said B2682, and preferably the activity is increased non-targeted.

Accordingly, in one embodiment, the molecule which activity is to be increased in the process of the invention is the gene product with an activity as a “b2682-protein”, preferably it is the molecule of section (a) or (b) of this paragraph.

In particular, it was observed that in plants, especially in Arabidopsis thaliana, increasing or generating the activity of a gene product non-targeted with the activity of a “b2682-protein”, preferably being encoded by a gene comprising the nucleic acid sequence SEQ ID NO. 115655, preferably the coding region thereof, conferred the production of or the increase in linolenic acid compared with the wild type control.

The nucleic acid sequence of B2790 from Escherichia coli, e.g. as shown in the respective line in column 5 of Table I, application no. 9, has been published in B. And the activity of the gene product thereof is the activity of flavodoxin.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “flavodoxin”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B2790, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B2790, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B2790, or a functional equivalent or a homolog thereof as depicted in column 8 of Table II, application no. 9, preferably a homolog or functional equivalent as depicted in column 8 of Table II B, application no. 9, and being depicted in the same respective line as said B2790, and preferably the activity is increased non-targeted.

Accordingly, in one embodiment, the molecule which activity is to be increased in the process of the invention is the gene product with an activity as a “flavodoxin”, preferably it is the molecule of section (a) or (b) of this paragraph.

In particular, it was observed that in plants, especially in Arabidopsis thaliana, increasing or generating the activity of a gene product non-targeted with the activity of a “flavodoxin”, preferably being encoded by a gene comprising the nucleic acid sequence SEQ ID NO. 115719, preferably the coding region thereof, conferred the production of or the increase in linolenic acid compared with the wild type control.

The nucleic acid sequence of B2836 from Escherichia coli, e.g. as shown in the respective line in column 5 of Table I, application no. 9, has been published in B. And the activity of the gene product thereof is the activity of AAS bifunctional protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “AAS bifunctional protein”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B2836, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B2836, and preferably the activity is increased plastidic, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B2836, or a functional equivalent or a homolog thereof as depicted in column 8 of Table II, application no. 9, preferably a homolog or functional equivalent as depicted in column 8 of Table II B, application no. 9, and being depicted in the same respective line as said B2836, and preferably the activity is increased plastidic.

Accordingly, in one embodiment, the molecule which activity is to be increased in the process of the invention is the gene product with an activity as a “AAS bifunctional protein”, preferably it is the molecule of section (a) or (b) of this paragraph.

In particular, it was observed that in plants, especially in Arabidopsis thaliana, increasing or generating the activity of a gene product plastidic with the activity of a “AAS bifunctional protein”, preferably being encoded by a gene comprising the nucleic acid sequence SEQ ID NO. 115791, preferably the coding region thereof, conferred the production of or the increase in linolenic acid compared with the wild type control.

The nucleic acid sequence of B3379 from Escherichia coli, e.g. as shown in the respective line in column 5 of Table I, application no. 9, has been published in B. And the activity of the gene product thereof is the activity of phosphotriesterase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “phosphotriesterase”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B3379, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B3379, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B3379, or a functional equivalent or a homolog thereof as depicted in column 8 of Table II, application no. 9, preferably a homolog or functional equivalent as depicted in column 8 of Table II B, application no. 9, and being depicted in the same respective line as said B3379, and preferably the activity is increased non-targeted.

Accordingly, in one embodiment, the molecule which activity is to be increased in the process of the invention is the gene product with an activity as a “phosphotriesterase”, preferably it is the molecule of section (a) or (b) of this paragraph.

In particular, it was observed that in plants, especially in Arabidopsis thaliana, increasing or generating the activity of a gene product non-targeted with the activity of a “phosphotriesterase”, preferably being encoded by a gene comprising the nucleic acid sequence SEQ ID NO. 116110, preferably the coding region thereof, conferred the production of or the increase in linolenic acid compared with the wild type control.

The nucleic acid sequence of B3442 from Escherichia coli, e.g. as shown in the respective line in column 5 of Table I, application no. 9, has been published in B. And the activity of the gene product thereof is the activity of b3442-protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “b3442-protein”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B3442, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B3442, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B3442, or a functional equivalent or a homolog thereof as depicted in column 8 of Table II, application no. 9, preferably a homolog or functional equivalent as depicted in column 8 of Table II B, application no. 9, and being depicted in the same respective line as said B3442, and preferably the activity is increased non-targeted.

Accordingly, in one embodiment, the molecule which activity is to be increased in the process of the invention is the gene product with an activity as a “b3442-protein”, preferably it is the molecule of section (a) or (b) of this paragraph.

In particular, it was observed that in plants, especially in Arabidopsis thaliana, increasing or generating the activity of a gene product non-targeted with the activity of a “b3442-protein”, preferably being encoded by a gene comprising the nucleic acid sequence SEQ ID NO. 94380, preferably the coding region thereof, conferred the production of or the increase in linolenic acid compared with the wild type control.

The nucleic acid sequence of B3534 from Escherichia coli, e.g. as shown in the respective line in column 5 of Table I, application no. 9, has been published in B. And the activity of the gene product thereof is the activity of b3534-protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “b3534-protein”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B3534, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B3534, and preferably the activity is increased plastidic, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B3534, or a functional equivalent or a homolog thereof as depicted in column 8 of Table II, application no. 9, preferably a homolog or functional equivalent as depicted in column 8 of Table II B, application no. 9, and being depicted in the same respective line as said B3534, and preferably the activity is increased plastidic.

Accordingly, in one embodiment, the molecule which activity is to be increased in the process of the invention is the gene product with an activity as a “b3534-protein”, preferably it is the molecule of section (a) or (b) of this paragraph.

In particular, it was observed that in plants, especially in Arabidopsis thaliana, increasing or generating the activity of a gene product plastidic with the activity of a “b3534-protein”, preferably being encoded by a gene comprising the nucleic acid sequence SEQ ID NO. 119181, preferably the coding region thereof, conferred the production of or the increase in linolenic acid compared with the wild type control.

The nucleic acid sequence of B3823 from Escherichia coli, e.g. as shown in the respective line in column 5 of Table I, application no. 9, has been published in B. And the activity of the gene product thereof is the activity of threonine efflux protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “threonine efflux protein”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B3823, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B3823, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B3823, or a functional equivalent or a homolog thereof as depicted in column 8 of Table II, application no. 9, preferably a homolog or functional equivalent as depicted in column 8 of Table II B, application no. 9, and being depicted in the same respective line as said B3823, and preferably the activity is increased non-targeted.

Accordingly, in one embodiment, the molecule which activity is to be increased in the process of the invention is the gene product with an activity as a “threonine efflux protein”, preferably it is the molecule of section (a) or (b) of this paragraph.

In particular, it was observed that in plants, especially in Arabidopsis thaliana, increasing or generating the activity of a gene product non-targeted with the activity of a “threonine efflux protein”, preferably being encoded by a gene comprising the nucleic acid sequence SEQ ID NO. 10726, preferably the coding region thereof, conferred the production of or the increase in linolenic acid compared with the wild type control.

The nucleic acid sequence of B4023 from Escherichia coli, e.g. as shown in the respective line in column 5 of Table I, application no. 9, has been published in B. And the activity of the gene product thereof is the activity of b4023-protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “b4023-protein”, especially from Escherichia coli or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said B4023, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said B4023, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said B4023, or a functional equivalent or a homolog thereof as depicted in column 8 of Table II, application no. 9, preferably a homolog or functional equivalent as depicted in column 8 of Table II B, application no. 9, and being depicted in the same respective line as said B4023, and preferably the activity is increased non-targeted.

Accordingly, in one embodiment, the molecule which activity is to be increased in the process of the invention is the gene product with an activity as a “b4023-protein”, preferably it is the molecule of section (a) or (b) of this paragraph.

In particular, it was observed that in plants, especially in Arabidopsis thaliana, increasing or generating the activity of a gene product non-targeted with the activity of a “b4023-protein”, preferably being encoded by a gene comprising the nucleic acid sequence SEQ ID NO. 119209, preferably the coding region thereof, conferred the production of or the increase in linolenic acid compared with the wild type control.

The nucleic acid sequence of GM02LC12622 from Glycine max, e.g. as shown in the respective line in column 5 of Table I, application no. 9, is unpublished. And the activity of the gene product thereof is the activity of glutaredoxin.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “glutaredoxin”, especially from Glycine max or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said GM02LC12622, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said GM02LC12622, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said GM02LC12622, or a functional equivalent or a homolog thereof as depicted in column 8 of Table II, application no. 9, preferably a homolog or functional equivalent as depicted in column 8 of Table II B, application no. 9, and being depicted in the same respective line as said GM02LC12622, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 10811, preferably the coding region thereof, conferred the production of or the increase in linolenic acid compared with the wild type control.

The nucleic acid sequence of GM02LC19289 from Glycine max, e.g. as shown in the respective line in column 5 of Table I, application no. 9, is unpublished. And the activity of the gene product thereof is the activity of transcription factor.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said GM02LC19289, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said GM02LC19289, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said GM02LC19289, or a functional equivalent or a homolog thereof as depicted in column 8 of Table II, application no. 9, preferably a homolog or functional equivalent as depicted in column 8 of Table II B, application no. 9, and being depicted in the same respective line as said GM02LC19289, and preferably the activity is increased non-targeted.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 46751, preferably the coding region thereof, conferred the production of or the increase in linolenic acid compared with the wild type control.

The nucleic acid sequence of Sll0374 from Synechocystis sp., e.g. as shown in the respective line in column 5 of Table I, application no. 9, has been published in C. And the activity of the gene product thereof is the activity of branched chain amino acid ABC transporter ATP-binding protein

Accordingly, in one embodiment, the process of the present invention for producing linolenic acid in a non-human organism, like a microorganism or a plant or a part thereof, comprises increasing or generating the activity of a gene product with the activity of a gene product conferring the activity of “branched chain amino acid ABC transporter ATP-binding protein”, especially from Synechocystis sp. or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Sll0374, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said Sll0374, and preferably the activity is increased plastidic, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Sll0374, or a functional equivalent or a homolog thereof as depicted in column 8 of Table II, application no. 9, preferably a homolog or functional equivalent as depicted in column 8 of Table II B, application no. 9, and being depicted in the same respective line as said Sll0374, and preferably the activity is increased plastidic.

Accordingly, in one embodiment, the molecule which activity is to be increased in the process of the invention is the gene product with an activity as a “branched chain amino acid ABC trans-porter ATP-binding protein”, preferably it is the molecule of section (a) or (b) of this paragraph.

In particular, it was observed that in plants, especially in Arabidopsis thaliana, increasing or generating the activity of a gene product plastidic with the activity of a “branched chain amino acid ABC transporter ATP-binding protein”, preferably being encoded by a gene comprising the nucleic acid sequence SEQ ID NO. 94741, preferably the coding region thereof, conferred the production of or the increase in linolenic acid compared with the wild type control.

The nucleic acid sequence of Sll0745 from Synechocystis sp., e.g. as shown in the respective line in column 5 of Table I, application no. 9, has been published in C. And the activity of the gene product thereof is the activity of phosphofructokinase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “phosphofructokinase”, especially from Synechocystis sp. or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Sll0745, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said Sll0745, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Sll0745, or a functional equivalent or a homolog thereof as depicted in column 8 of Table II, application no. 9, preferably a homolog or functional equivalent as depicted in column 8 of Table II B, application no. 9, and being depicted in the same respective line as said Sll0745, and preferably the activity is increased mitochondrial.

Accordingly, in one embodiment, the molecule which activity is to be increased in the process of the invention is the gene product with an activity as a “phosphofructokinase”, preferably it is the molecule of section (a) or (b) of this paragraph.

In particular, it was observed that in plants, especially in Arabidopsis thaliana, increasing or generating the activity of a gene product mitochondrial with the activity of a “phosphofructokinase”, preferably being encoded by a gene comprising the nucleic acid sequence SEQ ID NO. 112661, preferably the coding region thereof, conferred the production of or the increase in linolenic acid compared with the wild type control.

The nucleic acid sequence of Sll1031 from Synechocystis sp., e.g. as shown in the respective line in column 5 of Table I, application no. 9, has been published in C. And the activity of the gene product thereof is the activity of carbon dioxide concentrating mechanism protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “carbon dioxide concentrating mechanism protein”, especially from Synechocystis sp. or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Sll1031, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said Sll1031, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Sll1031, or a functional equivalent or a homolog thereof as depicted in column 8 of Table II, application no. 9, preferably a homolog or functional equivalent as depicted in column 8 of Table II B, application no. 9, and being depicted in the same respective line as said Sll1031, and preferably the activity is increased mitochondrial.

Accordingly, in one embodiment, the molecule which activity is to be increased in the process of the invention is the gene product with an activity as a “carbon dioxide concentrating mechanism protein”, preferably it is the molecule of section (a) or (b) of this paragraph.

In particular, it was observed that in plants, especially in Arabidopsis thaliana, increasing or generating the activity of a gene product mitochondrial with the activity of a “carbon dioxide concentrating mechanism protein”, preferably being encoded by a gene comprising the nucleic acid sequence SEQ ID NO. 52634, preferably the coding region thereof, conferred the production of or the increase in linolenic acid compared with the wild type control.

The nucleic acid sequence of Sll1032 from Synechocystis sp., e.g. as shown in the respective line in column 5 of Table I, application no. 9, has been published in C. And the activity of the gene product thereof is the activity of sll1032-protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “sll1032-protein”, especially from Synechocystis sp. or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Sll1032, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said Sll1032, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Sll1032, or a functional equivalent or a homolog thereof as depicted in column 8 of Table II, application no. 9, preferably a homolog or functional equivalent as depicted in column 8 of Table II B, application no. 9, and being depicted in the same respective line as said Sll1032, and preferably the activity is increased mitochondrial.

Accordingly, in one embodiment, the molecule which activity is to be increased in the process of the invention is the gene product with an activity as a “sll1032-protein”, preferably it is the molecule of section (a) or (b) of this paragraph.

In particular, it was observed that in plants, especially in Arabidopsis thaliana, increasing or generating the activity of a gene product mitochondrial with the activity of a “sll1032-protein”, preferably being encoded by a gene comprising the nucleic acid sequence SEQ ID NO. 85723, preferably the coding region thereof, conferred the production of or the increase in linolenic acid compared with the wild type control.

The nucleic acid sequence of Sll1443 from Synechocystis sp., e.g. as shown in the respective line in column 5 of Table I, application no. 9, has been published in C. And the activity of the gene product thereof is the activity of CTP synthetase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “CTP synthetase”, especially from Synechocystis sp. or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Sll1443, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said Sll1443, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Sll1443, or a functional equivalent or a homolog thereof as depicted in column 8 of Table II, application no. 9, preferably a homolog or functional equivalent as depicted in column 8 of Table II B, application no. 9, and being depicted in the same respective line as said Sll1443, and preferably the activity is increased mitochondrial.

Accordingly, in one embodiment, the molecule which activity is to be increased in the process of the invention is the gene product with an activity as a “CTP synthetase”, preferably it is the molecule of section (a) or (b) of this paragraph.

In particular, it was observed that in plants, especially in Arabidopsis thaliana, increasing or generating the activity of a gene product mitochondrial with the activity of a “CTP synthetase”, preferably being encoded by a gene comprising the nucleic acid sequence SEQ ID NO. 53878, preferably the coding region thereof, conferred the production of or the increase in linolenic acid compared with the wild type control.

The nucleic acid sequence of Sll1498 from Synechocystis sp., e.g. as shown in the respective line in column 5 of Table I, application no. 9, has been published in C. And the activity of the gene product thereof is the activity of carbamoyl-phosphate synthase subunit.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “carbamoyl-phosphate synthase subunit”, especially from Synechocystis sp. or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Sll1498, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said Sll1498, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Sll1498, or a functional equivalent or a homolog thereof as depicted in column 8 of Table II, application no. 9, preferably a homolog or functional equivalent as depicted in column 8 of Table II B, application no. 9, and being depicted in the same respective line as said Sll1498, and preferably the activity is increased non-targeted.

Accordingly, in one embodiment, the molecule which activity is to be increased in the process of the invention is the gene product with an activity as a “carbamoyl-phosphate synthase subunit”, preferably it is the molecule of section (a) or (b) of this paragraph.

In particular, it was observed that in plants, especially in Arabidopsis thaliana, increasing or generating the activity of a gene product non-targeted with the activity of a “carbamoyl-phosphate synthase subunit”, preferably being encoded by a gene comprising the nucleic acid sequence SEQ ID NO. 95047, preferably the coding region thereof, conferred the production of or the increase in linolenic acid compared with the wild type control.

The nucleic acid sequence of Sll1848 from Synechocystis sp., e.g. as shown in the respective line in column 5 of Table I, application no. 9, has been published in C. And the activity of the gene product thereof is the activity of acyl transferase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “acyl transferase”, especially from Synechocystis sp. or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Sll1848, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said Sll1848, and preferably the activity is increased plastidic, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Sll1848, or a functional equivalent or a homolog thereof as depicted in column 8 of Table II, application no. 9, preferably a homolog or functional equivalent as depicted in column 8 of Table II B, application no. 9, and being depicted in the same respective line as said Sll1848, and preferably the activity is increased plastidic.

Accordingly, in one embodiment, the molecule which activity is to be increased in the process of the invention is the gene product with an activity as a “acyl transferase”, preferably it is the molecule of section (a) or (b) of this paragraph.

In particular, it was observed that in plants, especially in Arabidopsis thaliana, increasing or generating the activity of a gene product plastidic with the activity of a “acyl transferase”, preferably being encoded by a gene comprising the nucleic acid sequence SEQ ID NO. 86447, preferably the coding region thereof, conferred the production of or the increase in linolenic acid compared with the wild type control.

The nucleic acid sequence of Sll1917 from Synechocystis sp., e.g. as shown in the respective line in column 5 of Table I, application no. 9, has been published in C. And the activity of the gene product thereof is the activity of coproporphyrinogen III oxidase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “coproporphyrinogen III oxidase”, especially from Synechocystis sp. or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Sll11917, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said Sll1917, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Sll1917, or a functional equivalent or a homolog thereof as depicted in column 8 of Table II, application no. 9, preferably a homolog or functional equivalent as depicted in column 8 of Table II B, application no. 9, and being depicted in the same respective line as said Sll1917, and preferably the activity is increased mitochondrial.

Accordingly, in one embodiment, the molecule which activity is to be increased in the process of the invention is the gene product with an activity as a “coproporphyrinogen III oxidase”, preferably it is the molecule of section (a) or (b) of this paragraph.

In particular, it was observed that in plants, especially in Arabidopsis thaliana, increasing or generating the activity of a gene product mitochondrial with the activity of a “coproporphyrinogen III oxidase”, preferably being encoded by a gene comprising the nucleic acid sequence SEQ ID NO. 11471, preferably the coding region thereof, conferred the production of or the increase in linolenic acid compared with the wild type control.

The nucleic acid sequence of Sll1920 from Synechocystis sp., e.g. as shown in the respective line in column 5 of Table I, application no. 9, has been published in C. And the activity of the gene product thereof is the activity of cation-transporting ATPase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “cation-transporting ATPase”, especially from Synechocystis sp. or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Sll1920,or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said SI11920, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Sll1920, or a functional equivalent or a homolog thereof as depicted in column 8 of Table II, application no. 9, preferably a homolog or functional equivalent as depicted in column 8 of Table II B, application no. 9, and being depicted in the same respective line as said Sll1920, and preferably the activity is increased mitochondrial.

Accordingly, in one embodiment, the molecule which activity is to be increased in the process of the invention is the gene product with an activity as a “cation-transporting ATPase”, preferably it is the molecule of section (a) or (b) of this paragraph.

In particular, it was observed that in plants, especially in Arabidopsis thaliana, increasing or generating the activity of a gene product mitochondrial with the activity of a “cation-transporting ATPase”, preferably being encoded by a gene comprising the nucleic acid sequence SEQ ID NO. 55385, preferably the coding region thereof, conferred the production of or the increase in linolenic acid compared with the wild type control.

The nucleic acid sequence of Slr0338 from Synechocystis sp., e.g. as shown in the respective line in column 5 of Table I, application no. 9, has been published in C. And the activity of the gene product thereof is the activity of oxidoreductase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “oxidoreductase”, especially from Synechocystis sp. or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Slr0338, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said Slr0338, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Slr0338, or a functional equivalent or a homolog thereof as depicted in column 8 of Table II, application no. 9, preferably a homolog or functional equivalent as depicted in column 8 of Table II B, application no. 9, and being depicted in the same respective line as said Slr0338, and preferably the activity is increased non-targeted.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 11990, preferably the coding region thereof, conferred the production of or the increase in linolenic acid compared with the wild type control.

The nucleic acid sequence of Slr0597 from Synechocystis sp., e.g. as shown in the respective line in column 5 of Table I, application no. 9, has been published in C. And the activity of the gene product thereof is the activity of bifunctional purine biosynthesis protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “bifunctional purine biosynthesis protein”, especially from Synechocystis sp. or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Slr0597, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said Slr0597, and preferably the activity is increased plastidic, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Slr0597, or a functional equivalent or a homolog thereof as depicted in column 8 of Table II, application no. 9, preferably a homolog or functional equivalent as depicted in column 8 of Table II B, application no. 9, and being depicted in the same respective line as said Slr0597, and preferably the activity is increased plastidic.

Accordingly, in one embodiment, the molecule which activity is to be increased in the process of the invention is the gene product with an activity as a “bifunctional purine biosynthesis protein”, preferably it is the molecule of section (a) or (b) of this paragraph.

In particular, it was observed that in plants, especially in Arabidopsis thaliana, increasing or generating the activity of a gene product plastidic with the activity of a “bifunctional purine biosynthesis protein”, preferably being encoded by a gene comprising the nucleic acid sequence SEQ ID NO. 56153, preferably the coding region thereof, conferred the production of or the increase in linolenic acid compared with the wild type control.

The nucleic acid sequence of Slr0949 from Synechocystis sp., e.g. as shown in the respective line in column 5 of Table I, application no. 9, has been published in C. And the activity of the gene product thereof is the activity of permease protein of ABC transporter.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “permease protein of ABC transporter”, especially from Synechocystis sp. or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Slr0949, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said Slr0949, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Slr0949, or a functional equivalent or a homolog thereof as depicted in column 8 of Table II, application no. 9, preferably a homolog or functional equivalent as depicted in column 8 of Table II B, application no. 9, and being depicted in the same respective line as said Slr0949, and preferably the activity is increased mitochondrial.

Accordingly, in one embodiment, the molecule which activity is to be increased in the process of the invention is the gene product with an activity as a “permease protein of ABC transporter”, preferably it is the molecule of section (a) or (b) of this paragraph.

In particular, it was observed that in plants, especially in Arabidopsis thaliana, increasing or generating the activity of a gene product mitochondrial with the activity of a “permease protein of ABC transporter”, preferably being encoded by a gene comprising the nucleic acid sequence SEQ ID NO. 119222, preferably the coding region thereof, conferred the production of or the increase in linolenic acid compared with the wild type control.

The nucleic acid sequence of Slr1099 from Synechocystis sp., e.g. as shown in the respective line in column 5 of Table I, application no. 9, has been published in C. And the activity of the gene product thereof is the activity of aromatic acid decarboxylase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “aromatic acid decarboxylase”, especially from Synechocystis sp. or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Slr1099, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said Slr1099, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Slr1099, or a functional equivalent or a homolog thereof as depicted in column 8 of Table II, application no. 9, preferably a homolog or functional equivalent as depicted in column 8 of Table II B, application no. 9, and being depicted in the same respective line as said Slr1099, and preferably the activity is increased mitochondrial.

Accordingly, in one embodiment, the molecule which activity is to be increased in the process of the invention is the gene product with an activity as a “aromatic acid decarboxylase”, preferably it is the molecule of section (a) or (b) of this paragraph.

In particular, it was observed that in plants, especially in Arabidopsis thaliana, increasing or generating the activity of a gene product mitochondrial with the activity of a “aromatic acid decarboxylase”, preferably being encoded by a gene comprising the nucleic acid sequence SEQ ID NO. 116341, preferably the coding region thereof, conferred the production of or the increase in linolenic acid compared with the wild type control.

The nucleic acid sequence of Slr1250 from Synechocystis sp., e.g. as shown in the respective line in column 5 of Table I, application no. 9, has been published in C. And the activity of the gene product thereof is the activity of phosphate import ATP-binding protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “phosphate import ATP-binding protein”, especially from Synechocystis sp. or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Slr1250, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said Slr1250, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Slr1250, or a functional equivalent or a homolog thereof as depicted in column 8 of Table II, application no. 9, preferably a homolog or functional equivalent as depicted in column 8 of Table II B, application no. 9, and being depicted in the same respective line as said Slr1250, and preferably the activity is increased mitochondrial.

Accordingly, in one embodiment, the molecule which activity is to be increased in the process of the invention is the gene product with an activity as a “phosphate import ATP-binding protein”, preferably it is the molecule of section (a) or (b) of this paragraph.

In particular, it was observed that in plants, especially in Arabidopsis thaliana, increasing or generating the activity of a gene product mitochondrial with the activity of a “phosphate import ATP-binding protein”, preferably being encoded by a gene comprising the nucleic acid sequence SEQ ID NO. 116460, preferably the coding region thereof, conferred the production of or the increase in linolenic acid compared with the wild type control.

The nucleic acid sequence of Slr1312 from Synechocystis sp., e.g. as shown in the respective line in column 5 of Table I, application no. 9, has been published in C. And the activity of the gene product thereof is the activity of arginine decarboxylase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “arginine decarboxylase”, especially from Synechocystis sp. or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Slr1312, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said Slr1312, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Slr1312, or a functional equivalent or a homolog thereof as depicted in column 8 of Table II, application no. 9, preferably a homolog or functional equivalent as depicted in column 8 of Table II B, application no. 9, and being depicted in the same respective line as said Slr1312, and preferably the activity is increased non-targeted.

Accordingly, in one embodiment, the molecule which activity is to be increased in the process of the invention is the gene product with an activity as a “arginine decarboxylase”, preferably it is the molecule of section (a) or (b) of this paragraph.

In particular, it was observed that in plants, especially in Arabidopsis thaliana, increasing or generating the activity of a gene product non-targeted with the activity of a “arginine decarboxylase”, preferably being encoded by a gene comprising the nucleic acid sequence SEQ ID NO. 58324, preferably the coding region thereof, conferred the production of or the increase in linolenic acid compared with the wild type control.

The nucleic acid sequence of Slr1791 from Synechocystis sp., e.g. as shown in the respective line in column 5 of Table I, application no. 9, has been published in C. And the activity of the gene product thereof is the activity of phosphoadenosine phosphosulfate reductase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “phosphoadenosine phosphosulfate reductase”, especially from Synechocystis sp. or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Slr1791, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said Slr1791, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Slr1791, or a functional equivalent or a homolog thereof as depicted in column 8 of Table II, application no. 9, preferably a homolog or functional equivalent as depicted in column 8 of Table II B, application no. 9, and being depicted in the same respective line as said Slr1791, and preferably the activity is increased mitochondrial.

Accordingly, in one embodiment, the molecule which activity is to be increased in the process of the invention is the gene product with an activity as a “phosphoadenosine phosphosulfate reductase”, preferably it is the molecule of section (a) or (b) of this paragraph.

In particular, it was observed that in plants, especially in Arabidopsis thaliana, increasing or generating the activity of a gene product mitochondrial with the activity of a “phosphoadenosine phosphosulfate reductase”, preferably being encoded by a gene comprising the nucleic acid sequence SEQ ID NO. 12140, preferably the coding region thereof, conferred the production of or the increase in linolenic acid compared with the wild type control.

The nucleic acid sequence of Slr2072 from Synechocystis sp., e.g. as shown in the respective line in column 5 of Table I, application no. 9, has been published in C. And the activity of the gene product thereof is the activity of threonine dehydratase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “threonine dehydratase”, especially from Synechocystis sp. or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Slr2072, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said Slr2072, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Slr2072, or a functional equivalent or a homolog thereof as depicted in column 8 of Table II, application no. 9, preferably a homolog or functional equivalent as depicted in column 8 of Table II B, application no. 9, and being depicted in the same respective line as said Slr2072, and preferably the activity is increased mitochondrial.

Accordingly, in one embodiment, the molecule which activity is to be increased in the process of the invention is the gene product with an activity as a “threonine dehydratase”, preferably it is the molecule of section (a) or (b) of this paragraph.

In particular, it was observed that in plants, especially in Arabidopsis thaliana, increasing or generating the activity of a gene product mitochondrial with the activity of a “threonine dehydratase”, preferably being encoded by a gene comprising the nucleic acid sequence SEQ ID NO. 12341, preferably the coding region thereof, conferred the production of or the increase in linolenic acid compared with the wild type control.

The nucleic acid sequence of TTC0917 from Thermus thermophilus, e.g. as shown in the respective line in column 5 of Table I, application no. 9, has been published in E. And the activity of the gene product thereof is the activity of metal-dependent hydrolase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “metal-dependent hydrolase”, especially from Thermus thermophilus or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said TTC0917, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said TTC0917, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said TTC0917, or a functional equivalent or a homolog thereof as depicted in column 8 of Table II, application no. 9, preferably a homolog or functional equivalent as depicted in column 8 of Table II B, application no. 9, and being depicted in the same respective line as said TTC0917, and preferably the activity is increased non-targeted.

Accordingly, in one embodiment, the molecule which activity is to be increased in the process of the invention is the gene product with an activity as a “metal-dependent hydrolase”, preferably it is the molecule of section (a) or (b) of this paragraph.

In particular, it was observed that in plants, especially in Arabidopsis thaliana, increasing or generating the activity of a gene product non-targeted with the activity of a “metal-dependent hydrolase”, preferably being encoded by a gene comprising the nucleic acid sequence SEQ ID NO. 61553, preferably the coding region thereof, conferred the production of or the increase in linolenic acid compared with the wild type control.

The nucleic acid sequence of TTC1386 from Thermus thermophilus, e.g. as shown in the respective line in column 5 of Table I, application no. 9, has been published in E. And the activity of the gene product thereof is the activity of TTC1386-protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “TTC1386-protein”, especially from Thermus thermophilus or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said TTC1386, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said TTC1386, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said TTC1386, or a functional equivalent or a homolog thereof as depicted in column 8 of Table II, application no. 9, preferably a homolog or functional equivalent as depicted in column 8 of Table II B, application no. 9, and being depicted in the same respective line as said TTC1386, and preferably the activity is increased non-targeted.

Accordingly, in one embodiment, the molecule which activity is to be increased in the process of the invention is the gene product with an activity as a “TTC1386-protein”, preferably it is the molecule of section (a) or (b) of this paragraph.

In particular, it was observed that in plants, especially in Arabidopsis thaliana, increasing or generating the activity of a gene product non-targeted with the activity of a “TTC1386-protein”, preferably being encoded by a gene comprising the nucleic acid sequence SEQ ID NO. 62079, preferably the coding region thereof, conferred the production of or the increase in linolenic acid compared with the wild type control.

The nucleic acid sequence of Ybr092c from Saccharomyces cerevisiae, e.g. as shown in the respective line in column 5 of Table I, application no. 9, has been published in A. And the activity of the gene product thereof is the activity of acid phosphatase precursor.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “acid phosphatase precursor”, especially from Saccharomyces cerevisiae or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Ybr092c, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said Ybr092c, and preferably the activity is increased plastidic, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Ybr092c, or a functional equivalent or a homolog thereof as depicted in column 8 of Table II, application no. 9, preferably a homolog or functional equivalent as depicted in column 8 of Table II B, application no. 9, and being depicted in the same respective line as said Ybr092c, and preferably the activity is increased plastidic.

Accordingly, in one embodiment, the molecule which activity is to be increased in the process of the invention is the gene product with an activity as a “acid phosphatase precursor”, preferably it is the molecule of section (a) or (b) of this paragraph.

In particular, it was observed that in plants, especially in Arabidopsis thaliana, increasing or generating the activity of a gene product plastidic with the activity of a “acid phosphatase precursor”, preferably being encoded by a gene comprising the nucleic acid sequence SEQ ID NO. 119347, preferably the coding region thereof, conferred the production of or the increase in linolenic acid compared with the wild type control.

The nucleic acid sequence of Ydl022w from Saccharomyces cerevisiae, e.g. as shown in the respective line in column 5 of Table I, application no. 9, has been published in A. And the activity of the gene product thereof is the activity of glycerol-3-phosphate dehydrogenase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “glycerol-3-phosphate dehydrogenase”, especially from Saccharomyces cerevisiae or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Ydl022w, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said Ydl022w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Ydl022w, or a functional equivalent or a homolog thereof as depicted in column 8 of Table II, application no. 9, preferably a homolog or functional equivalent as depicted in column 8 of Table II B, application no. 9, and being depicted in the same respective line as said Ydl022w, and preferably the activity is increased non-targeted.

Accordingly, in one embodiment, the molecule which activity is to be increased in the process of the invention is the gene product with an activity as a “glycerol-3-phosphate dehydrogenase”, preferably it is the molecule of section (a) or (b) of this paragraph.

In particular, it was observed that in plants, especially in Arabidopsis thaliana, increasing or generating the activity of a gene product non-targeted with the activity of a “glycerol-3-phosphate dehydrogenase”, preferably being encoded by a gene comprising the nucleic acid sequence SEQ ID NO. 119408, preferably the coding region thereof, conferred the production of or the increase in linolenic acid compared with the wild type control.

The nucleic acid sequence of Ydl126c from Saccharomyces cerevisiae, e.g. as shown in the respective line in column 5 of Table I, application no. 9, has been published in A. And the activity of the gene product thereof is the activity of cell division control protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “cell division control protein”, especially from Saccharomyces cerevisiae or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Ydl126c, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said Ydl126c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Ydl126c, or a functional equivalent or a homolog thereof as depicted in column 8 of Table II, application no. 9, preferably a homolog or functional equivalent as depicted in column 8 of Table II B, application no. 9, and being depicted in the same respective line as said Ydl126c, and preferably the activity is increased non-targeted.

Accordingly, in one embodiment, the molecule which activity is to be increased in the process of the invention is the gene product with an activity as a “cell division control protein”, preferably it is the molecule of section (a) or (b) of this paragraph.

In particular, it was observed that in plants, especially in Arabidopsis thaliana, increasing or generating the activity of a gene product non-targeted with the activity of a “cell division control protein”, preferably being encoded by a gene comprising the nucleic acid sequence SEQ ID NO. 120045, preferably the coding region thereof, conferred the production of or the increase in linolenic acid compared with the wild type control.

The nucleic acid sequence of Ydr046c from Saccharomyces cerevisiae, e.g. as shown in the respective line in column 5 of Table I, application no. 9, has been published in A. And the activity of the gene product thereof is the activity of branched-chain amino acid permease.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “branched-chain amino acid permease”, especially from Saccharomyces cerevisiae or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Ydr046c, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said Ydr046c, and preferably the activity is increased plastidic, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Ydr046c, or a functional equivalent or a homolog thereof as depicted in column 8 of Table II, application no. 9, preferably a homolog or functional equivalent as depicted in column 8 of Table II B, application no. 9, and being depicted in the same respective line as said Ydr046c, and preferably the activity is increased plastidic.

Accordingly, in one embodiment, the molecule which activity is to be increased in the process of the invention is the gene product with an activity as a “branched-chain amino acid permease”, preferably it is the molecule of section (a) or (b) of this paragraph.

In particular, it was observed that in plants, especially in Arabidopsis thaliana, increasing or generating the activity of a gene product plastidic with the activity of a “branched-chain amino acid permease”, preferably being encoded by a gene comprising the nucleic acid sequence SEQ ID NO. 63544, preferably the coding region thereof, conferred the production of or the increase in linolenic acid compared with the wild type control.

The nucleic acid sequence of Ydr131c from Saccharomyces cerevisiae, e.g. as shown in the respective line in column 5 of Table I, application no. 9, has been published in A. And the activity of the gene product thereof is the activity of F-box protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “F-box protein”, especially from Saccharomyces cerevisiae or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Ydr131c, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said Ydr131c, and preferably the activity is increased plastidic, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Ydr131c, or a functional equivalent or a homolog thereof as depicted in column 8 of Table II, application no. 9, preferably a homolog or functional equivalent as depicted in column 8 of Table II B, application no. 9, and being depicted in the same respective line as said Ydr131c, and preferably the activity is increased plastidic.

Accordingly, in one embodiment, the molecule which activity is to be increased in the process of the invention is the gene product with an activity as a “F-box protein”, preferably it is the molecule of section (a) or (b) of this paragraph.

In particular, it was observed that in plants, especially in Arabidopsis thaliana, increasing or generating the activity of a gene product plastidic with the activity of a “F-box protein”, preferably being encoded by a gene comprising the nucleic acid sequence SEQ ID NO. 14706, preferably the coding region thereof, conferred the production of or the increase in linolenic acid compared with the wild type control.

The nucleic acid sequence of Ydr339c from Saccharomyces cerevisiae, e.g. as shown in the respective line in column 5 of Table I, application no. 9, has been published in A. And the activity of the gene product thereof is the activity of ydr339c-protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “ydr339c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Ydr339c, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said Ydr339c, and preferably the activity is increased plastidic, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Ydr339c, or a functional equivalent or a homolog thereof as depicted in column 8 of Table II, application no. 9, preferably a homolog or functional equivalent as depicted in column 8 of Table II B, application no. 9, and being depicted in the same respective line as said Ydr339c, and preferably the activity is increased plastidic.

Accordingly, in one embodiment, the molecule which activity is to be increased in the process of the invention is the gene product with an activity as a “ydr339c-protein”, preferably it is the molecule of section (a) or (b) of this paragraph.

In particular, it was observed that in plants, especially in Arabidopsis thaliana, increasing or generating the activity of a gene product plastidic with the activity of a “ydr339c-protein”, preferably being encoded by a gene comprising the nucleic acid sequence SEQ ID NO. 120241, preferably the coding region thereof, conferred the production of or the increase in linolenic acid compared with the wild type control.

The nucleic acid sequence of Ydr507c from Saccharomyces cerevisiae, e.g. as shown in the respective line in column 5 of Table I, application no. 9, has been published in A. And the activity of the gene product thereof is the activity of ydr507c-protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “ydr507c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Ydr507c, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said Ydr507c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Ydr507c, or a functional equivalent or a homolog thereof as depicted in column 8 of Table II, application no. 9, preferably a homolog or functional equivalent as depicted in column 8 of Table II B, application no. 9, and being depicted in the same respective line as said Ydr507c, and preferably the activity is increased non-targeted.

Accordingly, in one embodiment, the molecule which activity is to be increased in the process of the invention is the gene product with an activity as a “ydr507c-protein”, preferably it is the molecule of section (a) or (b) of this paragraph.

In particular, it was observed that in plants, especially in Arabidopsis thaliana, increasing or generating the activity of a gene product non-targeted with the activity of a “ydr507c-protein”, preferably being encoded by a gene comprising the nucleic acid sequence SEQ ID NO. 90165, preferably the coding region thereof, conferred the production of or the increase in linolenic acid compared with the wild type control.

The nucleic acid sequence of Yfl053w from Saccharomyces cerevisiae, e.g. as shown in the respective line in column 5 of Table I, application no. 9, has been published in A. And the activity of the gene product thereof is the activity of dihydroxyacetone kinase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “dihydroxyacetone kinase”, especially from Saccharomyces cerevisiae or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Yfl053w, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said Yfl053w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Yfl053w, or a functional equivalent or a homolog thereof as depicted in column 8 of Table II, application no. 9, preferably a homolog or functional equivalent as depicted in column 8 of Table II B, application no. 9, and being depicted in the same respective line as said Yfl053w, and preferably the activity is increased non-targeted.

Accordingly, in one embodiment, the molecule which activity is to be increased in the process of the invention is the gene product with an activity as a “dihydroxyacetone kinase”, preferably it is the molecule of section (a) or (b) of this paragraph.

In particular, it was observed that in plants, especially in Arabidopsis thaliana, increasing or generating the activity of a gene product non-targeted with the activity of a “dihydroxyacetone kinase”, preferably being encoded by a gene comprising the nucleic acid sequence SEQ ID NO. 120309, preferably the coding region thereof, conferred the production of or the increase in linolenic acid compared with the wild type control.

The nucleic acid sequence of Ygl039w from Saccharomyces cerevisiae, e.g. as shown in the respective line in column 5 of Table I, application no. 9, has been published in A. And the activity of the gene product thereof is the activity of oxidoreductase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “oxidoreductase”, especially from Saccharomyces cerevisiae or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Ygl039w, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said Ygl039w, and preferably the activity is increased plastidic, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Ygl039w, or a functional equivalent or a homolog thereof as depicted in column 8 of Table II, application no. 9, preferably a homolog or functional equivalent as depicted in column 8 of Table II B, application no. 9, and being depicted in the same respective line as said Ygl039w, and preferably the activity is increased plastidic.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product plastidic with the activity of a “oxidoreductase”, preferably being encoded by a gene comprising the nucleic acid sequence SEQ ID NO. 120436, preferably the coding region thereof, conferred the production of or the increase in linolenic acid compared with the wild type control.

The nucleic acid sequence of Yjl073w from Saccharomyces cerevisiae, e.g. as shown in the respective line in column 5 of Table I, application no. 9, has been published in A. And the activity of the gene product thereof is the activity of DnaJ-like chaperone.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “DnaJ-like chaperone”, especially from Saccharomyces cerevisiae or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Yjl073w, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said Yjl073w, and preferably the activity is increased plastidic, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Yjl073w, or a functional equivalent or a homolog thereof as depicted in column 8 of Table II, application no. 9, preferably a homolog or functional equivalent as depicted in column 8 of Table II B, application no. 9, and being depicted in the same respective line as said Yjl073w, and preferably the activity is increased plastidic.

Accordingly, in one embodiment, the molecule which activity is to be increased in the process of the invention is the gene product with an activity as a “DnaJ-like chaperone”, preferably it is the molecule of section (a) or (b) of this paragraph.

In particular, it was observed that in plants, especially in Arabidopsis thaliana, increasing or generating the activity of a gene product plastidic with the activity of a “DnaJ-like chaperone”, preferably being encoded by a gene comprising the nucleic acid sequence SEQ ID NO. 64964, preferably the coding region thereof, conferred the production of or the increase in linolenic acid compared with the wild type control.

The nucleic acid sequence of Ymr013c from Saccharomyces cerevisiae, e.g. as shown in the respective line in column 5 of Table I, application no. 9, has been published in A. And the activity of the gene product thereof is the activity of ymr013c-protein.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “ymr013c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Ymr013c, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said Ymr013c, and preferably the activity is increased plastidic, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Ymr013c, or a functional equivalent or a homolog thereof as depicted in column 8 of Table II, application no. 9, preferably a homolog or functional equivalent as depicted in column 8 of Table II B, application no. 9, and being depicted in the same respective line as said Ymr013c, and preferably the activity is increased plastidic.

Accordingly, in one embodiment, the molecule which activity is to be increased in the process of the invention is the gene product with an activity as a “ymr013c-protein”, preferably it is the molecule of section (a) or (b) of this paragraph.

In particular, it was observed that in plants, especially in Arabidopsis thaliana, increasing or generating the activity of a gene product plastidic with the activity of a “ymr013c-protein”, preferably being encoded by a gene comprising the nucleic acid sequence SEQ ID NO. 91958, preferably the coding region thereof, conferred the production of or the increase in linolenic acid compared with the wild type control.

The nucleic acid sequence of Ynl064c from Saccharomyces cerevisiae, e.g. as shown in the respective line in column 5 of Table I, application no. 9, has been published in A. And the activity of the gene product thereof is the activity of molecular chaperone.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “molecular chaperone”, especially from Saccharomyces cerevisiae or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Ynl064c, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said Ynl064c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Ynl064c, or a functional equivalent or a homolog thereof as depicted in column 8 of Table II, application no. 9, preferably a homolog or functional equivalent as depicted in column 8 of Table II B, application no. 9, and being depicted in the same respective line as said Ynl064c, and preferably the activity is increased non-targeted.

Accordingly, in one embodiment, the molecule which activity is to be increased in the process of the invention is the gene product with an activity as a “molecular chaperone”, preferably it is the molecule of section (a) or (b) of this paragraph.

In particular, it was observed that in plants, especially in Arabidopsis thaliana, increasing or generating the activity of a gene product non-targeted with the activity of a “molecular chaperone”, preferably being encoded by a gene comprising the nucleic acid sequence SEQ ID NO. 117495, preferably the coding region thereof, conferred the production of or the increase in linolenic acid compared with the wild type control.

The nucleic acid sequence of Ypl271w from Saccharomyces cerevisiae, e.g. as shown in the respective line in column 5 of Table I, application no. 9, has been published in A. And the activity of the gene product thereof is the activity of ATPase epsilon subunit.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “ATPase epsilon subunit”, especially from Saccharomyces cerevisiae or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said Ypl271w, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said YpI271w, and preferably the activity is increased plastidic, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said Ypl271w, or a functional equivalent or a homolog thereof as depicted in column 8 of Table II, application no. 9, preferably a homolog or functional equivalent as depicted in column 8 of Table II B, application no. 9, and being depicted in the same respective line as said Ypl271w, and preferably the activity is increased plastidic.

Accordingly, in one embodiment, the molecule which activity is to be increased in the process of the invention is the gene product with an activity as a “ATPase epsilon subunit”, preferably it is the molecule of section (a) or (b) of this paragraph.

In particular, it was observed that in plants, especially in Arabidopsis thaliana, increasing or generating the activity of a gene product plastidic with the activity of a “ATPase epsilon subunit”, preferably being encoded by a gene comprising the nucleic acid sequence SEQ ID NO. 118212, preferably the coding region thereof, conferred the production of or the increase in linolenic acid compared with the wild type control.

The nucleic acid sequence of 59582753_SOYBEAN from Glycine max, e.g. as shown in the respective line in column 5 of Table I, application no. 9, is unpublished. And the activity of the gene product thereof is the activity of glucose-6-phosphate 1-dehydrogenase.

Accordingly, in one embodiment, the process of the present invention for producing linolenic acid in a non-human organism, like a microorganism or a plant or a part thereof, comprises increasing or generating the activity of a gene product with the activity of a gene product conferring the activity of “glucose-6-phosphate 1-dehydrogenase”, especially from Glycine max or its functional equivalent or its homolog, e.g. the increase of
(a) a gene product of a gene comprising the nucleic acid molecule as shown in the respective line in column 5 of Table I, application no. 9, preferably the coding region thereof, or a homolog or a fragment thereof, and being depicted in the same respective line as said 59582753_SOYBEAN, or a functional equivalent or a homolog thereof as shown in column 8 of Table I, application no. 9, preferably the coding region thereof, particularly a homolog or functional equivalent as shown in column 8 of Table I B, application no. 9, and being depicted in the same respective line as said 59582753_SOYBEAN, and preferably the activity is increased plastidic, or
(b) a polypeptide comprising a polypeptide, a consensus sequence or at least a polypeptide motif as shown in the respective line in column 5 of Table II or in column 8 of Table IV, application no. 9, respectively, and being depicted in the same respective line as said 59582753_SOYBEAN, or a functional equivalent or a homolog thereof as depicted in column 8 of Table II, application no. 9, preferably a homolog or functional equivalent as depicted in column 8 of Table II B, application no. 9, and being depicted in the same respective line as said 59582753_SOYBEAN, and preferably the activity is increased plastidic.

Accordingly, in one embodiment, the molecule which activity is to be increased in the process of the invention is the gene product with an activity as a “glucose-6-phosphate 1-dehydrogenase”, preferably it is the molecule of section (a) or (b) of this paragraph.

In particular, it was observed that in plants, especially in Arabidopsis thaliana, increasing or generating the activity of a gene product plastidic with the activity of a “glucose-6-phosphate 1-dehydrogenase”, preferably being encoded by a gene comprising the nucleic acid sequence SEQ ID NO. 16883, preferably the coding region thereof, conferred the production of or the increase in linolenic acid compared with the wild type control.

[0105.1.8.9] to [0107.1.8.9] for the disclosure of these paragraphs see [0105.1.8.8] to [0107.1.8.8] above.

A protein having an activity conferring an increase in the amount or level of the fine chemical linolenic acid, upon targeting to the plastids or mitochondria or upon non-targeting, preferably has the structure of the respective polypeptide described herein, in particular of the polypeptides comprising the consensus sequence or at least one polypeptide motifs as shown in the respective line in Table IV, application no. 9, column 8 or of the polypeptide comprising an amino acid sequence as disclosed in the respective line in Table II, application no. 9, columns 5 or 8, or homologs or fragments thereof as described herein, or is encoded by the nucleic acid molecule characterized herein or the nucleic acid molecule according to the invention, for example by the nucleic acid molecule as shown in the respective line in Table I, application no. 9, columns 5 or 8, preferably the coding region thereof, or homologs or fragments thereof and has the herein mentioned activity.

[0109.1.8.9] to [0110.1.8.9] for the disclosure of these paragraphs see [0109.1.8.8] to [0110.1.8.8] above.

In one embodiment, the process of the present invention comprises one or more of the following steps
(a) stabilizing a protein conferring the generation or increased expression of a protein encoded by the nucleic acid molecule of the invention or of the polypeptide of the invention, e.g. of a polypeptide having the activity selected from the group consisting of 3-oxoacyl-[acyl-carrier-protein] reductase, 3-oxoacyl-[acyl-carrier-protein] synthase, AAS bifunctional protein, acid phosphatase precursor, acid shock protein, acyl transferase, acyl-CoA synthase, arginine decarboxylase, aromatic acid decarboxylase, asparaginase, At2g45420-protein, ATPase epsilon subunit, b0195-protein, b1234-protein, b1248-protein, b1280-protein, b1330-protein, b1470-protein, b1670-protein, b1672-protein, b1837-protein, b2032-protein, b2107-protein, b2399-protein, b2474-protein, b2613-protein, b2682-protein, b3442-protein, b3534-protein, b4023-protein, bifunctional purine biosynthesis protein, branched chain amino acid ABC transporter ATP-binding protein, branched-chain amino acid permease, carbamoyl-phosphate synthase subunit, carbon dioxide concentrating mechanism protein, cation-transporting ATPase, cell division control protein, coproporphyrinogen III oxidase, CTP synthase, CTP synthetase, dihydroxyacetone kinase, DnaJ-like chaperone, electron transport complex protein, ethanolamine utilization protein, F-box protein, flavodoxin, fumarate hydratase, glucose-6-phosphate 1-dehydrogenase, glutaredoxin, glycerol-3-phosphate dehydrogenase, heat shock transcription factor, HesB/YadR/YfhF family protein, isopropylmalate isomerase large subunit, metal-dependent hydrolase, molecular chaperone, monothiol glutaredoxin, multidrug resistance protein, oxidoreductase, permease protein of ABC transporter, phosphate import ATP-binding protein, phosphoadenosine phosphosulfate reductase , phosphofructokinase, phosphotriesterase, protein phosphatase, pyruvate kinase, recombinase A, sec-independent protein translocase, sll1032-protein, ThiF family protein, threonine dehydratase, threonine efflux protein, transcription factor, TTC1386-protein, ydr339c-protein, ydr507c-protein, and ymr013c-protein, or of a polypeptide as indicated in the respective line in Table II, application no. 9, columns 5 or 8, or its homologs or fragments, and conferring the production of or an increase in linolenic acid, respectively; and/or
(b) stabilizing a mRNA conferring the generation or increased expression of a FCRP, e.g. protein encoded by the nucleic acid molecule of the invention or its homologs or fragments, or of a mRNA encoding the polypeptide of the present invention having the herein-mentioned activity selected from the group consisting of said activities mentioned in (a) and conferring the production of or an increase in linolenic acid, respectively; and/or
(c) increasing the specific activity of a protein conferring the increased expression of a FCRP, e.g. a protein encoded by the nucleic acid molecule of the invention or of the polypeptide of the present invention having herein-mentioned linolenic acid generating or increasing activity, respectively, e.g. of a polypeptide having the activity of a protein as indicated in the respective line in Table II, application no. 9, columns 5 or 8, or its homologs or fragments, or decreasing the inhibitory regulation of the polypeptide of the invention; and/or
(d) generating or increasing the expression of an endogenous or artificial transcription factor mediating the expression of a protein conferring the generation or increased expression of a FCRP, e.g. a protein encoded by the nucleic acid molecule of the invention or of the polypeptide of the invention having herein-mentioned activity selected from the group consisting of said activities mentioned in (a) and conferring a linolenic acid increasing activity, respectively, e.g. of a polypeptide having the activity of a protein as indicated in the respective line in Table II, application no. 9, columns 5 or 8 or its homologs; and/or
(e) stimulating activity of a protein conferring the increased expression of a FCRP, e.g. a protein encoded by the nucleic acid molecule of the present invention or a polypeptide of the present invention having herein-mentioned activity selected from the group consisting of said activities mentioned in (a) and conferring a linolenic acid increasing activity, respectively, e.g. of a polypeptide having the activity of a protein as indicated in the respective line in Table II, application no. 9, columns 5 or 8, or its homologs or fragments, by adding one or more exogenous inducing factors to the non-human organism or parts thereof; and/or
(f) expressing a transgenic gene encoding a protein conferring the increased expression of a FCRP, e.g. a polypeptide encoded by the nucleic acid molecule of the present invention or a polypeptide of the present invention, having herein-mentioned activity selected from the group consisting of said activities mentioned in (a) and conferring a linolenic acid increasing activity, respectively, e.g. of a polypeptide having the activity of a protein as indicated in the respective line in Table II, application no. 9, columns 5 or 8, or its homologs or fragments, and/or
(g) increasing the copy number of a gene conferring the increased expression of a nucleic acid molecule encoding a FCRP, e.g. a polypeptide encoded by the nucleic acid molecule of the invention or the polypeptide of the invention having herein-mentioned activity selected from the group consisting of said activities mentioned in (a) and conferring a linolenic acid increasing activity, respectively, e.g. of a polypeptide having the activity of a protein as indicated in the respective line in Table II, application no. 9, columns 5 or 8, or its homologs or fragments; and/or
(h) increasing the expression of the endogenous gene encoding the FCRP, polypeptide of the invention, e.g. a polypeptide encoded by the nucleic acid molecule of the invention or the polypeptide of the invention having herein-mentioned activity selected from the group consisting of said activities mentioned in (a) and conferring a linolenic acid; increasing activity, respectively, e.g. of a polypeptide having the activity of a protein as indicated in the respective line in Table II, application no. 9, columns 5 or 8, or its homologs or fragments, by adding positive expression or removing negative expression elements, e.g. homologous recombination can be used to either introduce positive regulatory elements like for plants the 35S enhancer into the promoter or to remove repressor elements form regulatory regions. Further gene conversion methods can be used to disrupt repressor elements or to enhance to activity of positive elements. Positive elements can be randomly introduced in plants by T-DNA or transposon mutagenesis and lines can be identified in which the positive elements have be integrated near to a gene of the invention, the expression of which is thereby enhanced; and/or
(i) modulating growth conditions of the non-human organism in such a manner, that the expression or activity of the gene encoding the FCRP, e.g. a protein of the invention or the protein itself is enhanced, for example microorganisms or plants can be grown for example under a higher temperature regime leading to an enhanced expression of heat shock proteins, which can lead to an enhanced linolenic acid production, respectively; and/or
(j) selecting of non-human organisms with especially high activity of the proteins of the invention from natural or from mutagenized resources and breeding them into the target organisms, e.g. the elite crops; and/or
(k) directing a protein encoded by the nucleic acid molecule of the invention or of the polypeptide of the present invention having herein-mentioned activity selected from the group consisting of said activities mentioned in (a) and conferring a linolenic acid increasing activity, respectively, e.g. of a polypeptide having the activity of a protein as indicated in the respective line in Table II, application no. 9, columns 5 or 8, or its homologs or fragments, if for the corresponding nucleic acid molecule in the respective line in column 6 of Table I the term “plastidic” is indicated, to the plastids by the addition of a plastidial targeting sequence or if for the corresponding nucleic acid molecule in the respective line in column 6 of Table I the term “mitochondrial” is indicated, to the mitochondria by the addition of a mitochondrial targeting sequence; and/or
(l) generating the expression of a protein encoded by the nucleic acid molecule of the invention or of the polypeptide of the present invention having herein-mentioned activity selected from the group consisting of said activities mentioned in (a) and conferring a linolenic acid increasing activity, respectively, e.g. of a polypeptide having the activity of a protein as indicated in the respective line in Table II, application no. 9, columns 5 or 8, or its homologs or fragments, if for the corresponding nucleic acid molecule in the respective line in column 6 of Table I the term “plastidic” or “mitochondrial” is indicated, in these organelles by the stable or transient transformation, advantageously stable transformation, of organelles, preferably plastids or mitochondria, with an inventive nucleic acid sequence preferably in form of an expression cassette containing said sequence leading to the expression of the nucleic acids or polypeptides of the invention in the respective organelle; and/or
(m) generating the expression of a protein encoded by the nucleic acid molecule of the invention or of the polypeptide of the present invention having herein-mentioned activity selected from the group consisting of said activities mentioned in (a) and conferring a linolenic acid increasing activity, respectively, e.g. of a polypeptide having the activity of a protein as indicated in the respective line in Table II, application no. 9, columns 5 or 8, or its homologs or fragments, if for the corresponding nucleic acid molecule in the respective line in column 6 of Table I the term “plastidic” or “mitochondrial” is indicated, in these organelles by integration of a nucleic acid of the invention into the genome of the respective organelle under control of preferable a promoter selective for the respective organelle.

Preferably, said mRNA is the coding region of the nucleic acid molecule of the present invention and/or the protein conferring the increased expression of a protein encoded by the coding region of the nucleic acid molecule of the present invention alone or linked to a transit nucleic acid sequence or transit peptide encoding nucleic acid sequence or the polypeptide having the herein mentioned activity, e.g. conferring the generation of or increase of linolenic acid, respectively, after increasing the expression or activity of the encoded polypeptide, non-targeted or in organelles such as plastids and/or mitochondria, preferably plastids, or having the activity of a polypeptide having an activity as the protein as shown in the respective line in Table II, application no. 9, column 3, or its homologs. Preferably the increase of linolenic acid, respectively, takes place non-targeted or in plastids and/or mitochondria, preferably non-targeted or in plastids.

[0113.1.8.9] to [0122.1.8.9] for the disclosure of these paragraphs see [0113.1.8.8] to [0122.1.8.8] above.

The activation of an endogenous polypeptide having above-mentioned activity, e.g. having the activity of a protein as shown in the respective line in Table II, application no. 9, column 3 or of the polypeptide of the invention, e.g. conferring the increase of the fine chemical linolenic acid, respectively, by increase of expression or activity in the cytoplasm, and/or in the cytosol, and/or in an organelle, such as plastids or mitochondria, can also be increased by introducing a synthetic transcription factor, which binds close to the coding region of the gene encoding the protein as shown in the respective line in Table II, application no. 9, column 5 or 8, or homologs or fragments thereof, and activates its transcription. A chimeric zinc finger protein can be constructed, which comprises a specific DNA-binding domain and an activation domain as e.g. the VP16 domain of Herpes Simplex virus. The specific binding domain can bind to the regulatory region of the gene encoding the protein as shown in the respective line in Table II, application no. 9, column 5 or 8, or homologs or fragments thereof. The expression of the chimeric transcription factor in a non-human organism, in particular in a plant, leads to a specific expression of the protein as shown in the respective line in Table II, application no. 9, column 5 or 8, or homologs or fragments thereof, see e.g. in WO01/52620, Oriz, Proc. Natl. Acad. Sci. USA 99, 13290 (2002), or Guan, Proc. Natl. Acad. Sci. USA 99, 13296 (2002).

[0124.1.8.9] to [0127.1.8.9] for the disclosure of these paragraphs see [0124.1.8.8] to [0127.1.8.8] above.

Owing to the introduction of a gene or a plurality of genes conferring the expression of the nucleic acid molecule of the invention or the polypeptide of the invention, for example the nucleic acid construct mentioned below, or encoding the protein as shown in the respective line in Table II, application no. 9, column 5 or 8, or homologs or fragments thereof, into a non-human organism alone or in combination with other genes, it is possible not only to increase the biosynthetic flux towards the end product, but also to increase, modify or create de novo an advantageous, preferably novel metabolites composition in the non-human organism, e.g. an advantageous composition comprising a higher content of (from a viewpoint of nutritional physiology limited) linolenic acid and if desired other fatty acids, and/or other metabolites, in free or bound form.

for the disclosure of this paragraph see [0129.1.8.8] above.

Accordingly, in one embodiment, the process according to the invention relates to a process, which comprises:
(a) providing a non-human organism, preferably a microorganism, a plant cell, a plant tissue, a plant or a part thereof, more preferably a microorganism, a plant tissue, a plant or a part thereof;
(b) increasing the activity of a protein as shown in the respective line in Table II, application no. 9, column 3 or of a polypeptide being encoded by the respective nucleic acid molecule of the present invention and described below, e.g. conferring an increase of the fine chemical linolenic acid, respectively, in the non-human organism, preferably in the microorganism, the plant cell, the plant tissue, the plant or a part thereof, more preferably a microorganism, a plant tissue, a plant or a part thereof, especially cytoplasmic or in an organelle, like plastids or mitochondria,
(c) growing the non-human organism, preferably the microorganism, the plant cell, the plant tissue, the plant or a part thereof, under conditions which permit the production of said fine chemical in the non-human organism, preferably the microorganism, the plant cell, the plant tissue, the plant or a part thereof; and
(d) if desired, recovering, optionally isolating, said fine chemical, in free and/or bound form, and, optionally further free and/or bound fatty acids, and/or other metabolites synthesized by the non-human organism, the microorganism, the plant cell, the plant tissue, the plant or a part thereof.

[0131.1.8.9] to [0139.1.8.9] for the disclosure of these paragraphs see [0131.1.8.8] to [0139.1.8.8] above.

In another embodiment, the invention relates to a (isolated) nucleic acid molecule, which comprises a nucleic acid molecule selected from the group consisting of:
(a) a nucleic acid molecule encoding a polypeptide shown in Table II, application no. 9, column 5 or 8, preferably shown in Table II B, application no. 9, column 8, or a homolog or a fragment thereof;
(b) a nucleic acid molecule shown in Table I, application no. 9, column 5 or 8, preferably shown in Table I B, application no. 9, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 9, preferably in column 8 of Table II B, application no. 9;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 9, preferably shown in column 8 of Table I B, application no. 9, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 9;
(i) a nucleic acid molecule encoding a polypeptide having the activity represented by a respective protein as depicted in column 5 of Table II, application no. 9;
(j) a nucleic acid molecule comprising a polynucleotide which is obtained by amplifying a cDNA library or a genomic library using the primers in Table III, application no. 9, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 9.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II application no. 9 and confers a generation or an increase of the respective fine chemical in a non-human organism or a part thereof as compared to a corresponding wild-type non-human organism or part thereof.

In another embodiment, the invention relates to a (isolated) nucleic acid molecule, which comprises a nucleic acid molecule selected from the group consisting of:
(a) a nucleic acid molecule encoding a polypeptide shown in Table II, column 5 or 8, application no. 9, preferably shown in Table II A, application no. 9, in column 5 or in Table II A, application no. 9, column 8 or in Table II B, application no. 9, column 8, or a homolog or a fragment thereof;
(b) a nucleic acid molecule shown in Table I, application no. 9, column 5 or 8, preferably shown in Table I A, application no. 9, in column 5 or in Table I A, application no. 9, column 8 or in Table I B, application no. 9, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 9, preferably shown in Table II A, application no. 9, in column 5 or in Table II A, application no. 9, column 8 or in Table II B, application no. 9, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 9, preferably shown in Table I A, application no. 9, in column 5 or in Table I A, application no. 9, column 8 or in Table I B column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 9;
(i) a nucleic acid molecule encoding a polypeptide having the activity represented by a respective protein as depicted in column 5 of Table II, application no. 9;
(j) a nucleic acid molecule comprising a polynucleotide which is obtained by amplifying a cDNA library or a genomic library using the primers in Table III, application no. 9, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt,500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto;
   whereby, preferably, the nucleic acid molecule according to (a), (b), (c), (d), (e), (f), (g), (h), (i), (j) or (k) distinguishes over the sequence depicted in Table I A and/or I B, application no. 9, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 9, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 9, column 5 or 8, or the coding region thereof, respectively. In a further embodiment the nucleic acid molecule does not encode the polypeptide sequence shown in Table II A and/or II B, application no. 9, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 9, column 5 or 8 and does not encode a protein of the sequence shown in Table II A and/or II B, application no. 9, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 9, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 9, column 5 or 8 but less than 100%, preferably less than 99.999%, 99.99%, 99.9%, 99%, 98%, 97%, 96% or 95% identical to the sequence shown in Table II A and/or II B, application no. 9, column 5 or 8.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 9.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II, application no. 9, and confers a generation or an increase of the respective fine chemical in a non-human organism or a part thereof as compared to a corresponding wild-type non-human organism or part thereof.

[0144.1.8.9] to [0155.1.8.9] for the disclosure of these paragraphs see [0144.1.8.8] to [0155.1.8.8] above.

In a preferred embodiment a nucleic acid construct, for example an expression cassette, comprises upstream, i.e. at the 5′ end of the encoding sequence, a promoter and downstream, i.e. at the 3′ end, a polyadenylation signal and optionally other regulatory elements which are operably linked to the intervening encoding sequence with one of the nucleic acids as depicted in the respective line in Table I, application no. 9, column 5 or 8, preferably the coding region thereof, or a homolog or a fragment thereof. By an operable linkage is meant the sequential arrangement of promoter, encoding sequence, terminator, optionally other regulatory elements and optionally targeting sequences in such a way that each of the regulatory elements/targeting sequence can fulfill its function in the expression of the encoding sequence in due manner. In one embodiment the sequences preferred for operable linkage are targeting sequences for ensuring subcellular localization in organelles, like plastids or mitochondria.

However, targeting sequences for ensuring subcellular localization in the endoplasmic reticulum (=ER), in the nucleus, in oil corpuscles or other compartments may also be employed as well as translation promoters such as the 5′ lead sequence in tobacco mosaic virus (Gallie et al., Nucl. Acids Res. 15, 8693 (1987)).

for the disclosure of this paragraph see [0157.1.8.8] above.

In an embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 9, column 5 or 8, preferably shown in Table II A, application no. 9, column 5, or in Table II A, application no. 9, column 8, or in Table II B, application no. 9, column 8, or a homolog or a fragment thereof;
   (b) a nucleic acid molecule shown in Table I, application no. 9, column 5 or 8, preferably shown in Table I A, application no. 9, column 5, or in Table I A, application no. 9, column 8, or in Table I B, application no. 9, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 9, preferably shown in Table II A, application no. 9, column 5, or in Table II A, application no. 9, column 8, or in Table II B, application no. 9, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 9, preferably shown in Table I A, application no. 9, column 5, or in Table I A, application no. 9, column 8, or in Table I B, application no. 9, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 9;
   (i) a nucleic acid molecule encoding a polypeptide having the activity represented by a respective protein as depicted in column 5 of Table II, application no. 9;
   (j) a nucleic acid molecule comprising a polynucleotide which is obtained by amplifying a cDNA library or a genomic library using the primers in Table III, application no. 9, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto. and, optionally
3) nucleic acid molecule encoding a transit peptide, preferably encoding a plastidal transit peptide or a mitochondrial transit peptide;
   which are operable linked.
   (All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 9.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 9 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 9, in the same line as of the respective nucleic acid molecule according to 2) “plastidic” is depicted, or encoding a mitochondrial transit peptide, especially in case in column 6 of Table I, application no. 9, in the same line as of the respective nucleic acid molecule according to 2) “mitochondrial” is depicted.

In another embodiment thereof the expression cassette does not comprise 3) a nucleic acid molecule encoding a transit peptide, especially in case in column 6 of Table I, application no. 9, in the same line as of the respective nucleic acid molecule according to 2) “non-targeted” is depicted.

In a further embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 9, column 5 or 8, preferably shown in Table II A, application no. 9, column 5, or in Table II A, application no. 9, column 8, or in Table II B, application no. 9, column 8, or a homolog or a fragment thereof;
   (b) a nucleic acid molecule shown in Table I, application no. 9, column 5 or 8, preferably shown in Table I A, application no. 9, column 5, or in Table I A, application no. 9, column 8, or in Table I B, application no. 9, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 9, preferably shown in Table II A, application no. 9, column 5, or in Table II A, application no. 9, column 8, or in Table II B, application no. 9, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 9, preferably shown in Table I A, application no. 9, column 5, or in Table I A, application no. 9, column 8, or in Table I B, application no. 9, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 9;
   (i) a nucleic acid molecule encoding a polypeptide having the activity represented by a respective protein as depicted in column 5 of Table II, application no. 9;
   (j) a nucleic acid molecule comprising a polynucleotide which is obtained by amplifying a cDNA library or a genomic library using the primers in Table III, application no. 9, column 8;
   and
      (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, optionally
3) nucleic acid molecule encoding a transit peptide, preferably encoding a plastidal transit peptide or a mitochondrial transit peptide;
   which are operable linked and
   whereby, preferably, the nucleic acid molecule according to 2 (a), (b), (c), (d), (e), (f), (g), (h), (i), (j) or (k) distinguishes over the sequence depicted in Table I A and/or I B, application no. 9, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention according to 2) does not consist of the sequence shown in Table I A and/or I B, application no. 9, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 9, column 5 or 8, or the coding region thereof, respectively. In a fur-ther embodiment the nucleic acid molecule does not encode the polypeptide sequence shown in Table II A and/or II B, application no. 9, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 9, column 5 or 8 and does not encode a protein of the sequence shown in Table II A and/or II B, application no. 9, column 5 or 8. Accordingly, in one embodiment, the protein encoded by a sequence of a nucleic acid according to 2(a), (b), (c), (d), (e), (f), (g), (h), (i), (j) or (k) does not consist of the sequence shown in Table I A and/or I B, application no. 9, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 9, column 5 or 8 but less than 100%, 99.999%, 99.99%, 99.9%, 99%, 98%, 97%, 96% or 95% identical to the sequence shown in Table II A and/or II B, application no. 9, column 5 or 8.
   (All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 9.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 9 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 9, in the same line as of the respective nucleic acid molecule according to 2) “plastidic” is depicted, or encoding a mitochondrial transit peptide, especially in case in column 6 of Table I, application no. 9, in the same line as of the respective nucleic acid molecule according to 2) “mitochondrial” is depicted.

In another embodiment thereof the expression cassette does not comprise 3) a nucleic acid molecule encoding a transit peptide, especially in case in column 6 of Table I, application no. 9, in the same line as of the respective nucleic acid molecule according to 2) “non-targeted” is depicted.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17451, or a homolog or fragment thereof, or a homolog thereof being depicted in Table I, application no. 9, column 8, in the same line as SEQ ID NO. 17451, preferably the coding region thereof, a ho-molog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 17451 is depicted, plastidic is mentioned, or a mitochondrial transit peptide in case in Table I, application no. 9, in column 6 in the same line as SEQ ID NO. 17451 is depicted, mitochondrial is mentioned; which is operable linked, too.

In another embodiment thereof the expression cassette does not comprise an additional transit peptide in case in Table I, application no. 9, in column 6 in the same line as SEQ ID NO. 17451 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 1061, or a homolog or fragment thereof, or a homolog thereof being depicted in Table I, application no. 9, column 8, in the same line as SEQ ID NO. 1061, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 1061 is depicted, plastidic is mentioned, or a mitochondrial transit peptide in case in Table I, application no. 9, in column 6 in the same line as SEQ ID NO. 1061 is depicted, mitochondrial is mentioned; which is operable linked, too.

In another embodiment thereof the expression cassette does not comprise an additional transit peptide in case in Table I, application no. 9, in column 6 in the same line as SEQ ID NO. 1061 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 19419, or a homolog or fragment thereof, or a homolog thereof being depicted in Table I, application no. 9, column 8, in the same line as SEQ ID NO. 19419, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 19419 is depicted, plastidic is mentioned, or a mitochondrial transit peptide in case in Table I, application no. 9, in column 6 in the same line as SEQ ID NO. 19419 is depicted, mitochondrial is mentioned; which is operable linked, too. In another embodiment thereof the expression cassette does not comprise an additional transit peptide in case in Table I, application no. 9, in column 6 in the same line as SEQ ID NO. 19419 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 113714, or a homolog or fragment thereof, or a homolog thereof being depicted in Table I, application no. 9, column 8, in the same line as SEQ ID NO. 113714, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 113714 is depicted, plastidic is mentioned, or a mitochondrial transit peptide in case in Table I, application no. 9, in column 6 in the same line as SEQ ID NO. 113714 is depicted, mitochondrial is mentioned; which is operable linked, too.

In another embodiment thereof the expression cassette does not comprise an additional transit peptide in case in Table I, application no. 9, in column 6 in the same line as SEQ ID NO. 113714 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 1623, or a homolog or fragment thereof, or a homolog thereof being depicted in Table I, application no. 9, column 8, in the same line as SEQ ID NO. 1623, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 1623 is depicted, plastidic is mentioned, or a mitochondrial transit peptide in case in Table I, application no. 9, in column 6 in the same line as SEQ ID NO. 1623 is depicted, mitochondrial is mentioned; which is operable linked, too.

In another embodiment thereof the expression cassette does not comprise an additional transit peptide in case in Table I, application no. 9, in column 6 in the same line as SEQ ID NO. 1623 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68657, or a homolog or fragment thereof, or a homolog thereof being depicted in Table I, application no. 9, column 8, in the same line as SEQ ID NO. 68657, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 68657 is depicted, plastidic is mentioned, or a mitochondrial transit peptide in case in Table I, application no. 9, in column 6 in the same line as SEQ ID NO. 68657 is depicted, mitochondrial is mentioned; which is operable linked, too.

In another embodiment thereof the expression cassette does not comprise an additional transit peptide in case in Table I, application no. 9, in column 6 in the same line as SEQ ID NO. 68657 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 2935, or a homolog or fragment thereof, or a homolog thereof being depicted in Table I, application no. 9, column 8, in the same line as SEQ ID NO. 2935, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 2935 is depicted, plastidic is mentioned, or a mitochondrial transit peptide in case in Table I, application no. 9, in column 6 in the same line as SEQ ID NO. 2935 is depicted, mitochondrial is mentioned; which is operable linked, too.

In another embodiment thereof the expression cassette does not comprise an additional transit peptide in case in Table I, application no. 9, in column 6 in the same line as SEQ ID NO. 2935 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 3279, or a homolog or fragment thereof, or a homolog thereof being depicted in Table I, application no. 9, column 8, in the same line as SEQ ID NO. 3279, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 3279 is depicted, plastidic is mentioned, or a mitochondrial transit peptide in case in Table I, application no. 9, in column 6 in the same line as SEQ ID NO. 3279 is depicted, mitochondrial is mentioned; which is operable linked, too.

In another embodiment thereof the expression cassette does not comprise an additional transit peptide in case in Table I, application no. 9, in column 6 in the same line as SEQ ID NO. 3279 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 24232, or a homolog or fragment thereof, or a homolog thereof being depicted in Table I, application no. 9, column 8, in the same line as SEQ ID NO. 24232, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 24232 is depicted, plastidic is mentioned, or a mitochondrial transit peptide in case in Table I, application no. 9, in column 6 in the same line as SEQ ID NO. 24232 is depicted, mitochondrial is mentioned; which is operable linked, too.

In another embodiment thereof the expression cassette does not comprise an additional transit peptide in case in Table I, application no. 9, in column 6 in the same line as SEQ ID NO. 24232 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68849, or a homolog or fragment thereof, or a homolog thereof being depicted in Table I, application no. 9, column 8, in the same line as SEQ ID NO. 68849, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 68849 is depicted, plastidic is mentioned, or a mitochondrial transit peptide in case in Table I, application no. 9, in column 6 in the same line as SEQ ID NO. 68849 is depicted, mitochondrial is mentioned; which is operable linked, too.

In another embodiment thereof the expression cassette does not comprise an additional transit peptide in case in Table I, application no. 9, in column 6 in the same line as SEQ ID NO. 68849 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4102, or a homolog or fragment thereof, or a homolog thereof being depicted in Table I, application no. 9, column 8, in the same line as SEQ ID NO. 4102, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 4102 is depicted, plastidic is mentioned, or a mitochondrial transit peptide in case in Table I, application no. 9, in column 6 in the same line as SEQ ID NO. 4102 is depicted, mitochondrial is mentioned; which is operable linked, too.

In another embodiment thereof the expression cassette does not comprise an additional transit peptide in case in Table I, application no. 9, in column 6 in the same line as SEQ ID NO. 4102 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 24438, or a homolog or fragment thereof, or a homolog thereof being depicted in Table I, application no. 9, column 8, in the same line as SEQ ID NO. 24438, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 24438 is depicted, plastidic is mentioned, or a mitochondrial transit peptide in case in Table I, application no. 9, in column 6 in the same line as SEQ ID NO. 24438 is depicted, mitochondrial is mentioned; which is operable linked, too.

In another embodiment thereof the expression cassette does not comprise an additional transit peptide in case in Table I, application no. 9, in column 6 in the same line as SEQ ID NO. 24438 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4904, or a homolog or fragment thereof, or a homolog thereof being depicted in Table I, application no. 9, column 8, in the same line as SEQ ID NO. 4904, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 4904 is depicted, plastidic is mentioned, or a mitochondrial transit peptide in case in Table I, application no. 9, in column 6 in the same line as SEQ ID NO. 4904 is depicted, mitochondrial is mentioned; which is operable linked, too.

In another embodiment thereof the expression cassette does not comprise an additional transit peptide in case in Table I, application no. 9, in column 6 in the same line as SEQ ID NO. 4904 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 5318, or a homolog or fragment thereof, or a homolog thereof being depicted in Table I, application no. 9, column 8, in the same line as SEQ ID NO. 5318, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 5318 is depicted, plastidic is mentioned, or a mitochondrial transit peptide in case in Table I, application no. 9, in column 6 in the same line as SEQ ID NO. 5318 is depicted, mitochondrial is mentioned; which is operable linked, too.

In another embodiment thereof the expression cassette does not comprise an additional transit peptide in case in Table I, application no. 9, in column 6 in the same line as SEQ ID NO. 5318 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 25498, or a homolog or fragment thereof, or a homolog thereof being depicted in Table I, application no. 9, column 8, in the same line as SEQ ID NO. 25498, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 25498 is depicted, plastidic is mentioned, or a mitochondrial transit peptide in case in Table I, application no. 9, in column 6 in the same line as SEQ ID NO. 25498 is depicted, mitochondrial is mentioned; which is operable linked, too.

In another embodiment thereof the expression cassette does not comprise an additional transit peptide in case in Table I, application no. 9, in column 6 in the same line as SEQ ID NO. 25498 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 26196, or a homolog or fragment thereof, or a homolog thereof being depicted in Table I, application no. 9, column 8, in the same line as SEQ ID NO. 26196, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 26196 is depicted, plastidic is mentioned, or a mitochondrial transit peptide in case in Table I, application no. 9, in column 6 in the same line as SEQ ID NO. 26196 is depicted, mitochondrial is mentioned; which is operable linked, too.

In another embodiment thereof the expression cassette does not comprise an additional transit peptide in case in Table I, application no. 9, in column 6 in the same line as SEQ ID NO. 26196 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 114083, or a homolog or fragment thereof, or a homolog thereof being depicted in Table I, application no. 9, column 8, in the same line as SEQ ID NO. 114083, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 114083 is depicted, plastidic is mentioned, or a mitochondrial transit peptide in case in Table I, application no. 9, in column 6 in the same line as SEQ ID NO. 114083 is depicted, mitochondrial is mentioned; which is operable linked, too.

In another embodiment thereof the expression cassette does not comprise an additional transit peptide in case in Table I, application no. 9, in column 6 in the same line as SEQ ID NO. 114083 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 28040, or a homolog or fragment thereof, or a homolog thereof being depicted in Table I, application no. 9, column 8, in the same line as SEQ ID NO. 28040, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 28040 is depicted, plastidic is mentioned, or a mitochondrial transit peptide in case in Table I, application no. 9, in column 6 in the same line as SEQ ID NO. 28040 is depicted, mitochondrial is mentioned; which is operable linked, too.

In another embodiment thereof the expression cassette does not comprise an additional transit peptide in case in Table I, application no. 9, in column 6 in the same line as SEQ ID NO. 28040 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 29500, or a homolog or fragment thereof, or a homolog thereof being depicted in Table I, application no. 9, column 8, in the same line as SEQ ID NO. 29500, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 29500 is depicted, plastidic is mentioned, or a mitochondrial transit peptide in case in Table I, application no. 9, in column 6 in the same line as SEQ ID NO. 29500 is depicted, mitochondrial is mentioned; which is operable linked, too.

In another embodiment thereof the expression cassette does not comprise an additional transit peptide in case in Table I, application no. 9, in column 6 in the same line as SEQ ID NO. 29500 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 111155, or a homolog or fragment thereof, or a homolog thereof being depicted in Table I, application no. 9, column 8, in the same line as SEQ ID NO. 111155, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 111155 is depicted, plastidic is mentioned, or a mitochondrial transit peptide in case in Table I, application no. 9, in column 6 in the same line as SEQ ID NO. 111155 is depicted, mitochondrial is mentioned; which is operable linked, too.

In another embodiment thereof the expression cassette does not comprise an additional transit peptide in case in Table I, application no. 9, in column 6 in the same line as SEQ ID NO. 111155 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 31026, or a homolog or fragment thereof, or a homolog thereof being depicted in Table I, application no. 9, column 8, in the same line as SEQ ID NO. 31026, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 31026 is depicted, plastidic is mentioned, or a mitochondrial transit peptide in case in Table I, application no. 9, in column 6 in the same line as SEQ ID NO. 31026 is depicted, mitochondrial is mentioned; which is operable linked, too.

In another embodiment thereof the expression cassette does not comprise an additional transit peptide in case in Table I, application no. 9, in column 6 in the same line as SEQ ID NO. 31026 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 114231, or a homolog or fragment thereof, or a homolog thereof being depicted in Table I, application no. 9, column 8, in the same line as SEQ ID NO. 114231, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 114231 is depicted, plastidic is mentioned, or a mitochondrial transit peptide in case in Table I, application no. 9, in column 6 in the same line as SEQ ID NO. 114231 is depicted, mitochondrial is mentioned; which is operable linked, too.

In another embodiment thereof the expression cassette does not comprise an additional transit peptide in case in Table I, application no. 9, in column 6 in the same line as SEQ ID NO. 114231 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 102941, or a homolog or fragment thereof, or a homolog thereof being depicted in Table I, application no. 9, column 8, in the same line as SEQ ID NO. 102941, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 102941 is depicted, plastidic is mentioned, or a mitochondrial transit peptide in case in Table I, application no. 9, in column 6 in the same line as SEQ ID NO. 102941 is depicted, mitochondrial is mentioned; which is operable linked, too.

In another embodiment thereof the expression cassette does not comprise an additional transit peptide in case in Table I, application no. 9, in column 6 in the same line as SEQ ID NO. 102941 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 32648, or a homolog or fragment thereof, or a homolog thereof being depicted in Table I, application no. 9, column 8, in the same line as SEQ ID NO. 32648, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 32648 is depicted, plastidic is mentioned, or a mitochondrial transit peptide in case in Table I, application no. 9, in column 6 in the same line as SEQ ID NO. 32648 is depicted, mitochondrial is mentioned; which is operable linked, too.

In another embodiment thereof the expression cassette does not comprise an additional transit peptide in case in Table I, application no. 9, in column 6 in the same line as SEQ ID NO. 32648 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 118238, or a homolog or fragment thereof, or a homolog thereof being depicted in Table I, application no. 9, column 8, in the same line as SEQ ID NO. 118238, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 118238 is depicted, plastidic is mentioned, or a mitochondrial transit peptide in case in Table I, application no. 9, in column 6 in the same line as SEQ ID NO. 118238 is depicted, mitochondrial is mentioned; which is operable linked, too.

In another embodiment thereof the expression cassette does not comprise an additional transit peptide in case in Table I, application no. 9, in column 6 in the same line as SEQ ID NO. 118238 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 36299, or a homolog or fragment thereof, or a homolog thereof being depicted in Table I, application no. 9, column 8, in the same line as SEQ ID NO. 36299, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 36299 is depicted, plastidic is mentioned, or a mitochondrial transit peptide in case in Table I, application no. 9, in column 6 in the same line as SEQ ID NO. 36299 is depicted, mitochondrial is mentioned; which is operable linked, too.

In another embodiment thereof the expression cassette does not comprise an additional transit peptide in case in Table I, application no. 9, in column 6 in the same line as SEQ ID NO. 36299 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 76032, or a homolog or fragment thereof, or a homolog thereof being depicted in Table I, application no. 9, column 8, in the same line as SEQ ID NO. 76032, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 76032 is depicted, plastidic is mentioned, or a mitochondrial transit peptide in case in Table I, application no. 9, in column 6 in the same line as SEQ ID NO. 76032 is depicted, mitochondrial is mentioned; which is operable linked, too.

In another embodiment thereof the expression cassette does not comprise an additional transit peptide in case in Table I, application no. 9, in column 6 in the same line as SEQ ID NO. 76032 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 118371, or a homolog or fragment thereof, or a homolog thereof being depicted in Table I, application no. 9, column 8, in the same line as SEQ ID NO. 118371, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 118371 is depicted, plastidic is mentioned, or a mitochondrial transit peptide in case in Table I, application no. 9, in column 6 in the same line as SEQ ID NO. 118371 is depicted, mitochondrial is mentioned; which is operable linked, too.

In another embodiment thereof the expression cassette does not comprise an additional transit peptide in case in Table I, application no. 9, in column 6 in the same line as SEQ ID NO. 118371 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 114552, or a homolog or fragment thereof, or a homolog thereof being depicted in Table I, application no. 9, column 8, in the same line as SEQ ID NO. 114552, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 114552 is depicted, plastidic is mentioned, or a mitochondrial transit peptide in case in Table I, application no. 9, in column 6 in the same line as SEQ ID NO. 114552 is depicted, mitochondrial is mentioned; which is operable linked, too.

In another embodiment thereof the expression cassette does not comprise an additional transit peptide in case in Table I, application no. 9, in column 6 in the same line as SEQ ID NO. 114552 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 112111, or a homolog or fragment thereof, or a homolog thereof being depicted in Table I, application no. 9, column 8, in the same line as SEQ ID NO. 112111, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 112111 is depicted, plastidic is mentioned, or a mitochondrial transit peptide in case in Table I, application no. 9, in column 6 in the same line as SEQ ID NO. 112111 is depicted, mitochondrial is mentioned; which is operable linked, too.

In another embodiment thereof the expression cassette does not comprise an additional transit peptide in case in Table I, application no. 9, in column 6 in the same line as SEQ ID NO. 112111 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 119135, or a homolog or fragment thereof, or a homolog thereof being depicted in Table I, application no. 9, column 8, in the same line as SEQ ID NO. 119135, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 119135 is depicted, plastidic is mentioned, or a mitochondrial transit peptide in case in Table I, application no. 9, in column 6 in the same line as SEQ ID NO. 119135 is depicted, mitochondrial is mentioned; which is operable linked, too.

In another embodiment thereof the expression cassette does not comprise an additional transit peptide in case in Table I, application no. 9, in column 6 in the same line as SEQ ID NO. 119135 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 37573, or a homolog or fragment thereof, or a homolog thereof being depicted in Table I, application no. 9, column 8, in the same line as SEQ ID NO. 37573, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 37573 is depicted, plastidic is mentioned, or a mitochondrial transit peptide in case in Table I, application no. 9, in column 6 in the same line as SEQ ID NO. 37573 is depicted, mitochondrial is mentioned; which is operable linked, too.

In another embodiment thereof the expression cassette does not comprise an additional transit peptide in case in Table I, application no. 9, in column 6 in the same line as SEQ ID NO. 37573 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38226, or a homolog or fragment thereof, or a homolog thereof being depicted in Table I, application no. 9, column 8, in the same line as SEQ ID NO. 38226, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 38226 is depicted, plastidic is mentioned, or a mitochondrial transit peptide in case in Table I, application no. 9, in column 6 in the same line as SEQ ID NO. 38226 is depicted, mitochondrial is mentioned; which is operable linked, too.

In another embodiment thereof the expression cassette does not comprise an additional transit peptide in case in Table I, application no. 9, in column 6 in the same line as SEQ ID NO. 38226 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115056, or a homolog or fragment thereof, or a homolog thereof being depicted in Table I, application no. 9, column 8, in the same line as SEQ ID NO. 115056, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 115056 is depicted, plastidic is mentioned, or a mitochondrial transit peptide in case in Table I, application no. 9, in column 6 in the same line as SEQ ID NO. 115056 is depicted, mitochondrial is mentioned; which is operable linked, too.

In another embodiment thereof the expression cassette does not comprise an additional transit peptide in case in Table I, application no. 9, in column 6 in the same line as SEQ ID NO. 115056 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38300, or a homolog or fragment thereof, or a homolog thereof being depicted in Table I, application no. 9, column 8, in the same line as SEQ ID NO. 38300, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 38300 is depicted, plastidic is mentioned, or a mitochondrial transit peptide in case in Table I, application no. 9, in column 6 in the same line as SEQ ID NO. 38300 is depicted, mitochondrial is mentioned; which is operable linked, too.

In another embodiment thereof the expression cassette does not comprise an additional transit peptide in case in Table I, application no. 9, in column 6 in the same line as SEQ ID NO. 38300 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115064, or a homolog or fragment thereof, or a homolog thereof being depicted in Table I, application no. 9, column 8, in the same line as SEQ ID NO. 115064, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 115064 is depicted, plastidic is mentioned, or a mitochondrial transit peptide in case in Table I, application no. 9, in column 6 in the same line as SEQ ID NO. 115064 is depicted, mitochondrial is mentioned; which is operable linked, too.

In another embodiment thereof the expression cassette does not comprise an additional transit peptide in case in Table I, application no. 9, in column 6 in the same line as SEQ ID NO. 115064 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38345, or a homolog or fragment thereof, or a homolog thereof being depicted in Table I, application no. 9, column 8, in the same line as SEQ ID NO. 38345, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 38345 is depicted, plastidic is mentioned, or a mitochondrial transit peptide in case in Table I, application no. 9, in column 6 in the same line as SEQ ID NO. 38345 is depicted, mitochondrial is mentioned; which is operable linked, too.

In another embodiment thereof the expression cassette does not comprise an additional transit peptide in case in Table I, application no. 9, in column 6 in the same line as SEQ ID NO. 38345 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78753, or a homolog or fragment thereof, or a homolog thereof being depicted in Table I, application no. 9, column 8, in the same line as SEQ ID NO. 78753, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 78753 is depicted, plastidic is mentioned, or a mitochondrial transit peptide in case in Table I, application no. 9, in column 6 in the same line as SEQ ID NO. 78753 is depicted, mitochondrial is mentioned; which is operable linked, too.

In another embodiment thereof the expression cassette does not comprise an additional transit peptide in case in Table I, application no. 9, in column 6 in the same line as SEQ ID NO. 78753 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78771, or a homolog or fragment thereof, or a homolog thereof being depicted in Table I, application no. 9, column 8, in the same line as SEQ ID NO. 78771, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 78771 is depicted, plastidic is mentioned, or a mitochondrial transit peptide in case in Table I, application no. 9, in column 6 in the same line as SEQ ID NO. 78771 is depicted, mitochondrial is mentioned; which is operable linked, too.

In another embodiment thereof the expression cassette does not comprise an additional transit peptide in case in Table I, application no. 9, in column 6 in the same line as SEQ ID NO. 78771 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78953, or a homolog or fragment thereof, or a homolog thereof being depicted in Table I, application no. 9, column 8, in the same line as SEQ ID NO. 78953, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 78953 is depicted, plastidic is mentioned, or a mitochondrial transit peptide in case in Table I, application no. 9, in column 6 in the same line as SEQ ID NO. 78953 is depicted, mitochondrial is mentioned; which is operable linked, too.

In another embodiment thereof the expression cassette does not comprise an additional transit peptide in case in Table I, application no. 9, in column 6 in the same line as SEQ ID NO. 78953 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 8920, or a homolog or fragment thereof, or a homolog thereof being depicted in Table I, application no. 9, column 8, in the same line as SEQ ID NO. 8920, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 8920 is depicted, plastidic is mentioned, or a mitochondrial transit peptide in case in Table I, application no. 9, in column 6 in the same line as SEQ ID NO. 8920 is depicted, mitochondrial is mentioned; which is operable linked, too.

In another embodiment thereof the expression cassette does not comprise an additional transit peptide in case in Table I, application no. 9, in column 6 in the same line as SEQ ID NO. 8920 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39002, or a homolog or fragment thereof, or a homolog thereof being depicted in Table I, application no. 9, column 8, in the same line as SEQ ID NO. 39002, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 39002 is depicted, plastidic is mentioned, or a mitochondrial transit peptide in case in Table I, application no. 9, in column 6 in the same line as SEQ ID NO. 39002 is depicted, mitochondrial is mentioned; which is operable linked, too.

In another embodiment thereof the expression cassette does not comprise an additional transit peptide in case in Table I, application no. 9, in column 6 in the same line as SEQ ID NO. 39002 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39237, or a homolog or fragment thereof, or a homolog thereof being depicted in Table I, application no. 9, column 8, in the same line as SEQ ID NO. 39237, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 39237 is depicted, plastidic is mentioned, or a mitochondrial transit peptide in case in Table I, application no. 9, in column 6 in the same line as SEQ ID NO. 39237 is depicted, mitochondrial is mentioned; which is operable linked, too.

In another embodiment thereof the expression cassette does not comprise an additional transit peptide in case in Table I, application no. 9, in column 6 in the same line as SEQ ID NO. 39237 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115628, or a homolog or fragment thereof, or a homolog thereof being depicted in Table I, application no. 9, column 8, in the same line as SEQ ID NO. 115628, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 115628 is depicted, plastidic is mentioned, or a mitochondrial transit peptide in case in Table I, application no. 9, in column 6 in the same line as SEQ ID NO. 115628 is depicted, mitochondrial is mentioned; which is operable linked, too.

In another embodiment thereof the expression cassette does not comprise an additional transit peptide in case in Table I, application no. 9, in column 6 in the same line as SEQ ID NO. 115628 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40329, or a homolog or fragment thereof, or a homolog thereof being depicted in Table I, application no. 9, column 8, in the same line as SEQ ID NO. 40329, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 40329 is depicted, plastidic is mentioned, or a mitochondrial transit peptide in case in Table I, application no. 9, in column 6 in the same line as SEQ ID NO. 40329 is depicted, mitochondrial is mentioned; which is operable linked, too.

In another embodiment thereof the expression cassette does not comprise an additional transit peptide in case in Table I, application no. 9, in column 6 in the same line as SEQ ID NO. 40329 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40665, or a homolog or fragment thereof, or a homolog thereof being depicted in Table I, application no. 9, column 8, in the same line as SEQ ID NO. 40665, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 40665 is depicted, plastidic is mentioned, or a mitochondrial transit peptide in case in Table I, application no. 9, in column 6 in the same line as SEQ ID NO. 40665 is depicted, mitochondrial is mentioned; which is operable linked, too.

In another embodiment thereof the expression cassette does not comprise an additional transit peptide in case in Table I, application no. 9, in column 6 in the same line as SEQ ID NO. 40665 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115655, or a homolog or fragment thereof, or a homolog thereof being depicted in Table I, application no. 9, column 8, in the same line as SEQ ID NO. 115655, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 115655 is depicted, plastidic is mentioned, or a mitochondrial transit peptide in case in Table I, application no. 9, in column 6 in the same line as SEQ ID NO. 115655 is depicted, mitochondrial is mentioned; which is operable linked, too.

In another embodiment thereof the expression cassette does not comprise an additional transit peptide in case in Table I, application no. 9, in column 6 in the same line as SEQ ID NO. 115655 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115719, or a homolog or fragment thereof, or a homolog thereof being depicted in Table I, application no. 9, column 8, in the same line as SEQ ID NO. 115719, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 115719 is depicted, plastidic is mentioned, or a mitochondrial transit peptide in case in Table I, application no. 9, in column 6 in the same line as SEQ ID NO. 115719 is depicted, mitochondrial is mentioned; which is operable linked, too.

In another embodiment thereof the expression cassette does not comprise an additional transit peptide in case in Table I, application no. 9, in column 6 in the same line as SEQ ID NO. 115719 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115791, or a homolog or fragment thereof, or a homolog thereof being depicted in Table I, application no. 9, column 8, in the same line as SEQ ID NO. 115791, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 115791 is depicted, plastidic is mentioned, or a mitochondrial transit peptide in case in Table I, application no. 9, in column 6 in the same line as SEQ ID NO. 115791 is depicted, mitochondrial is mentioned; which is operable linked, too.

In another embodiment thereof the expression cassette does not comprise an additional transit peptide in case in Table I, application no. 9, in column 6 in the same line as SEQ ID NO. 115791 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 116110, or a homolog or fragment thereof, or a homolog thereof being depicted in Table I, application no. 9, column 8, in the same line as SEQ ID NO. 116110, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 116110 is depicted, plastidic is mentioned, or a mitochondrial transit peptide in case in Table I, application no. 9, in column 6 in the same line as SEQ ID NO. 116110 is depicted, mitochondrial is mentioned; which is operable linked, too.

In another embodiment thereof the expression cassette does not comprise an additional transit peptide in case in Table I, application no. 9, in column 6 in the same line as SEQ ID NO. 116110 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 94380, or a homolog or fragment thereof, or a homolog thereof being depicted in Table I, application no. 9, column 8, in the same line as SEQ ID NO. 94380, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 94380 is depicted, plastidic is mentioned, or a mitochondrial transit peptide in case in Table I, application no. 9, in column 6 in the same line as SEQ ID NO. 94380 is depicted, mitochondrial is mentioned; which is operable linked, too.

In another embodiment thereof the expression cassette does not comprise an additional transit peptide in case in Table I, application no. 9, in column 6 in the same line as SEQ ID NO. 94380 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 119181, or a homolog or fragment thereof, or a homolog thereof being depicted in Table I, application no. 9, column 8, in the same line as SEQ ID NO. 119181, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 119181 is depicted, plastidic is mentioned, or a mitochondrial transit peptide in case in Table I, application no. 9, in column 6 in the same line as SEQ ID NO. 119181 is depicted, mitochondrial is mentioned; which is operable linked, too.

In another embodiment thereof the expression cassette does not comprise an additional transit peptide in case in Table I, application no. 9, in column 6 in the same line as SEQ ID NO. 119181 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 10726, or a homolog or fragment thereof, or a homolog thereof being depicted in Table I, application no. 9, column 8, in the same line as SEQ ID NO. 10726, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 10726 is depicted, plastidic is mentioned, or a mitochondrial transit peptide in case in Table I, application no. 9, in column 6 in the same line as SEQ ID NO. 10726 is depicted, mitochondrial is mentioned; which is operable linked, too.

In another embodiment thereof the expression cassette does not comprise an additional transit peptide in case in Table I, application no. 9, in column 6 in the same line as SEQ ID NO. 10726 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 119209, or a homolog or fragment thereof, or a homolog thereof being depicted in Table I, application no. 9, column 8, in the same line as SEQ ID NO. 119209, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 119209 is depicted, plastidic is mentioned, or a mitochondrial transit peptide in case in Table I, application no. 9, in column 6 in the same line as SEQ ID NO. 119209 is depicted, mitochondrial is mentioned; which is operable linked, too.

In another embodiment thereof the expression cassette does not comprise an additional transit peptide in case in Table I, application no. 9, in column 6 in the same line as SEQ ID NO. 119209 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 10811, or a homolog or fragment thereof, or a homolog thereof being depicted in Table I, application no. 9, column 8, in the same line as SEQ ID NO. 10811, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 10811 is depicted, plastidic is mentioned, or a mitochondrial transit peptide in case in Table I, application no. 9, in column 6 in the same line as SEQ ID NO. 10811 is depicted, mitochondrial is mentioned; which is operable linked, too.

In another embodiment thereof the expression cassette does not comprise an additional transit peptide in case in Table I, application no. 9, in column 6 in the same line as SEQ ID NO. 10811 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 46751, or a homolog or fragment thereof, or a homolog thereof being depicted in Table I, application no. 9, column 8, in the same line as SEQ ID NO. 46751, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 46751 is depicted, plastidic is mentioned, or a mitochondrial transit peptide in case in Table I, application no. 9, in column 6 in the same line as SEQ ID NO. 46751 is depicted, mitochondrial is mentioned; which is operable linked, too.

In another embodiment thereof the expression cassette does not comprise an additional transit peptide in case in Table I, application no. 9, in column 6 in the same line as SEQ ID NO. 46751 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 94741, or a homolog or fragment thereof, or a homolog thereof being depicted in Table I, application no. 9, column 8, in the same line as SEQ ID NO. 94741, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 94741 is depicted, plastidic is mentioned, or a mitochondrial transit peptide in case in Table I, application no. 9, in column 6 in the same line as SEQ ID NO. 94741 is depicted, mitochondrial is mentioned; which is operable linked, too.

In another embodiment thereof the expression cassette does not comprise an additional transit peptide in case in Table I, application no. 9, in column 6 in the same line as SEQ ID NO. 94741 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 112661, or a homolog or fragment thereof, or a homolog thereof being depicted in Table I, application no. 9, column 8, in the same line as SEQ ID NO. 112661, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 112661 is depicted, plastidic is mentioned, or a mitochondrial transit peptide in case in Table I, application no. 9, in column 6 in the same line as SEQ ID NO. 112661 is depicted, mitochondrial is mentioned; which is operable linked, too.

In another embodiment thereof the expression cassette does not comprise an additional transit peptide in case in Table I, application no. 9, in column 6 in the same line as SEQ ID NO. 112661 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 52634, or a homolog or fragment thereof, or a homolog thereof being depicted in Table I, application no. 9, column 8, in the same line as SEQ ID NO. 52634, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 52634 is depicted, plastidic is mentioned, or a mitochondrial transit peptide in case in Table I, application no. 9, in column 6 in the same line as SEQ ID NO. 52634 is depicted, mitochondrial is mentioned; which is operable linked, too.

In another embodiment thereof the expression cassette does not comprise an additional transit peptide in case in Table I, application no. 9, in column 6 in the same line as SEQ ID NO. 52634 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 85723, or a homolog or fragment thereof, or a homolog thereof being depicted in Table I, application no. 9, column 8, in the same line as SEQ ID NO. 85723, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 85723 is depicted, plastidic is mentioned, or a mitochondrial transit peptide in case in Table I, application no. 9, in column 6 in the same line as SEQ ID NO. 85723 is depicted, mitochondrial is mentioned; which is operable linked, too.

In another embodiment thereof the expression cassette does not comprise an additional transit peptide in case in Table I, application no. 9, in column 6 in the same line as SEQ ID NO. 85723 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 53878, or a homolog or fragment thereof, or a homolog thereof being depicted in Table I, application no. 9, column 8, in the same line as SEQ ID NO. 53878, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 53878 is depicted, plastidic is mentioned, or a mitochondrial transit peptide in case in Table I, application no. 9, in column 6 in the same line as SEQ ID NO. 53878 is depicted, mitochondrial is mentioned; which is operable linked, too.

In another embodiment thereof the expression cassette does not comprise an additional transit peptide in case in Table I, application no. 9, in column 6 in the same line as SEQ ID NO. 53878 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 95047, or a homolog or fragment thereof, or a homolog thereof being depicted in Table I, application no. 9, column 8, in the same line as SEQ ID NO. 95047, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 95047 is depicted, plastidic is mentioned, or a mitochondrial transit peptide in case in Table I, application no. 9, in column 6 in the same line as SEQ ID NO. 95047 is depicted, mitochondrial is mentioned; which is operable linked, too.

In another embodiment thereof the expression cassette does not comprise an additional transit peptide in case in Table I, application no. 9, in column 6 in the same line as SEQ ID NO. 95047 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 86447, or a homolog or fragment thereof, or a homolog thereof being depicted in Table I, application no. 9, column 8, in the same line as SEQ ID NO. 86447, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 86447 is depicted, plastidic is mentioned, or a mitochondrial transit peptide in case in Table I, application no. 9, in column 6 in the same line as SEQ ID NO. 86447 is depicted, mitochondrial is mentioned; which is operable linked, too.

In another embodiment thereof the expression cassette does not comprise an additional transit peptide in case in Table I, application no. 9, in column 6 in the same line as SEQ ID NO. 86447 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 11471, or a homolog or fragment thereof, or a homolog thereof being depicted in Table I, application no. 9, column 8, in the same line as SEQ ID NO. 11471, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 11471 is depicted, plastidic is mentioned, or a mitochondrial transit peptide in case in Table I, application no. 9, in column 6 in the same line as SEQ ID NO. 11471 is depicted, mitochondrial is mentioned; which is operable linked, too.

In another embodiment thereof the expression cassette does not comprise an additional transit peptide in case in Table I, application no. 9, in column 6 in the same line as SEQ ID NO. 11471 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 55385, or a homolog or fragment thereof, or a homolog thereof being depicted in Table I, application no. 9, column 8, in the same line as SEQ ID NO. 55385, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 55385 is depicted, plastidic is mentioned, or a mitochondrial transit peptide in case in Table I, application no. 9, in column 6 in the same line as SEQ ID NO. 55385 is depicted, mitochondrial is mentioned; which is operable linked, too.

In another embodiment thereof the expression cassette does not comprise an additional transit peptide in case in Table I, application no. 9, in column 6 in the same line as SEQ ID NO. 55385 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 11990, or a homolog or fragment thereof, or a homolog thereof being depicted in Table I, application no. 9, column 8, in the same line as SEQ ID NO. 11990, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 11990 is depicted, plastidic is mentioned, or a mitochondrial transit peptide in case in Table I, application no. 9, in column 6 in the same line as SEQ ID NO. 11990 is depicted, mitochondrial is mentioned; which is operable linked, too.

In another embodiment thereof the expression cassette does not comprise an additional transit peptide in case in Table I, application no. 9, in column 6 in the same line as SEQ ID NO. 11990 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 56153, or a homolog or fragment thereof, or a homolog thereof being depicted in Table I, application no. 9, column 8, in the same line as SEQ ID NO. 56153, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 56153 is depicted, plastidic is mentioned, or a mitochondrial transit peptide in case in Table I, application no. 9, in column 6 in the same line as SEQ ID NO. 56153 is depicted, mitochondrial is mentioned; which is operable linked, too.

In another embodiment thereof the expression cassette does not comprise an additional transit peptide in case in Table I, application no. 9, in column 6 in the same line as SEQ ID NO. 56153 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 119222, or a homolog or fragment thereof, or a homolog thereof being depicted in Table I, application no. 9, column 8, in the same line as SEQ ID NO. 119222, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 119222 is depicted, plastidic is mentioned, or a mitochondrial transit peptide in case in Table I, application no. 9, in column 6 in the same line as SEQ ID NO. 119222 is depicted, mitochondrial is mentioned; which is operable linked, too.

In another embodiment thereof the expression cassette does not comprise an additional transit peptide in case in Table I, application no. 9, in column 6 in the same line as SEQ ID NO. 119222 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 116341, or a homolog or fragment thereof, or a homolog thereof being depicted in Table I, application no. 9, column 8, in the same line as SEQ ID NO. 116341, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 116341 is depicted, plastidic is mentioned, or a mitochondrial transit peptide in case in Table I, application no. 9, in column 6 in the same line as SEQ ID NO. 116341 is depicted, mitochondrial is mentioned; which is operable linked, too.

In another embodiment thereof the expression cassette does not comprise an additional transit peptide in case in Table I, application no. 9, in column 6 in the same line as SEQ ID NO. 116341 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 116460, or a homolog or fragment thereof, or a homolog thereof being depicted in Table I, application no. 9, column 8, in the same line as SEQ ID NO. 116460, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 116460 is depicted, plastidic is mentioned, or a mitochondrial transit peptide in case in Table I, application no. 9, in column 6 in the same line as SEQ ID NO. 116460 is depicted, mitochondrial is mentioned; which is operable linked, too.

In another embodiment thereof the expression cassette does not comprise an additional transit peptide in case in Table I, application no. 9, in column 6 in the same line as SEQ ID NO. 116460 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 58324, or a homolog or fragment thereof, or a homolog thereof being depicted in Table I, application no. 9, column 8, in the same line as SEQ ID NO. 58324, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 58324 is depicted, plastidic is mentioned, or a mitochondrial transit peptide in case in Table I, application no. 9, in column 6 in the same line as SEQ ID NO. 58324 is depicted, mitochondrial is mentioned; which is operable linked, too.

In another embodiment thereof the expression cassette does not comprise an additional transit peptide in case in Table I, application no. 9, in column 6 in the same line as SEQ ID NO. 58324 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12140, or a homolog or fragment thereof, or a homolog thereof being depicted in Table I, application no. 9, column 8, in the same line as SEQ ID NO. 12140, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 12140 is depicted, plastidic is mentioned, or a mitochondrial transit peptide in case in Table I, application no. 9, in column 6 in the same line as SEQ ID NO. 12140 is depicted, mitochondrial is mentioned; which is operable linked, too.

In another embodiment thereof the expression cassette does not comprise an additional transit peptide in case in Table I, application no. 9, in column 6 in the same line as SEQ ID NO. 12140 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12341, or a homolog or fragment thereof, or a homolog thereof being depicted in Table I, application no. 9, column 8, in the same line as SEQ ID NO. 12341, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 12341 is depicted, plastidic is mentioned, or a mitochondrial transit peptide in case in Table I, application no. 9, in column 6 in the same line as SEQ ID NO. 12341 is depicted, mitochondrial is mentioned; which is operable linked, too.

In another embodiment thereof the expression cassette does not comprise an additional transit peptide in case in Table I, application no. 9, in column 6 in the same line as SEQ ID NO. 12341 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 61553, or a homolog or fragment thereof, or a homolog thereof being depicted in Table I, application no. 9, column 8, in the same line as SEQ ID NO. 61553, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 61553 is depicted, plastidic is mentioned, or a mitochondrial transit peptide in case in Table I, application no. 9, in column 6 in the same line as SEQ ID NO. 61553 is depicted, mitochondrial is mentioned; which is operable linked, too.

In another embodiment thereof the expression cassette does not comprise an additional transit peptide in case in Table I, application no. 9, in column 6 in the same line as SEQ ID NO. 61553 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 62079, or a homolog or fragment thereof, or a homolog thereof being depicted in Table I, application no. 9, column 8, in the same line as SEQ ID NO. 62079, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 62079 is depicted, plastidic is mentioned, or a mitochondrial transit peptide in case in Table I, application no. 9, in column 6 in the same line as SEQ ID NO. 62079 is depicted, mitochondrial is mentioned; which is operable linked, too.

In another embodiment thereof the expression cassette does not comprise an additional transit peptide in case in Table I, application no. 9, in column 6 in the same line as SEQ ID NO. 62079 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 119347, or a homolog or fragment thereof, or a homolog thereof being depicted in Table I, application no. 9, column 8, in the same line as SEQ ID NO. 119347, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 119347 is depicted, plastidic is mentioned, or a mitochondrial transit peptide in case in Table I, application no. 9, in column 6 in the same line as SEQ ID NO. 119347 is depicted, mitochondrial is mentioned; which is operable linked, too.

In another embodiment thereof the expression cassette does not comprise an additional transit peptide in case in Table I, application no. 9, in column 6 in the same line as SEQ ID NO. 119347 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 119408, or a homolog or fragment thereof, or a homolog thereof being depicted in Table I, application no. 9, column 8, in the same line as SEQ ID NO. 119408, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 119408 is depicted, plastidic is mentioned, or a mitochondrial transit peptide in case in Table I, application no. 9, in column 6 in the same line as SEQ ID NO. 119408 is depicted, mitochondrial is mentioned; which is operable linked, too.

In another embodiment thereof the expression cassette does not comprise an additional transit peptide in case in Table I, application no. 9, in column 6 in the same line as SEQ ID NO. 119408 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 120045, or a homolog or fragment thereof, or a homolog thereof being depicted in Table I, application no. 9, column 8, in the same line as SEQ ID NO. 120045, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 120045 is depicted, plastidic is mentioned, or a mitochondrial transit peptide in case in Table I, application no. 9, in column 6 in the same line as SEQ ID NO. 120045 is depicted, mitochondrial is mentioned; which is operable linked, too.

In another embodiment thereof the expression cassette does not comprise an additional transit peptide in case in Table I, application no. 9, in column 6 in the same line as SEQ ID NO. 120045 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 63544, or a homolog or fragment thereof, or a homolog thereof being depicted in Table I, application no. 9, column 8, in the same line as SEQ ID NO. 63544, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 63544 is depicted, plastidic is mentioned, or a mitochondrial transit peptide in case in Table I, application no. 9, in column 6 in the same line as SEQ ID NO. 63544 is depicted, mitochondrial is mentioned; which is operable linked, too.

In another embodiment thereof the expression cassette does not comprise an additional transit peptide in case in Table I, application no. 9, in column 6 in the same line as SEQ ID NO. 63544 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 14706, or a homolog or fragment thereof, or a homolog thereof being depicted in Table I, application no. 9, column 8, in the same line as SEQ ID NO. 14706, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 14706 is depicted, plastidic is mentioned, or a mitochondrial transit peptide in case in Table I, application no. 9, in column 6 in the same line as SEQ ID NO. 14706 is depicted, mitochondrial is mentioned; which is operable linked, too.

In another embodiment thereof the expression cassette does not comprise an additional transit peptide in case in Table I, application no. 9, in column 6 in the same line as SEQ ID NO. 14706 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 120241, or a homolog or fragment thereof, or a homolog thereof being depicted in Table I, application no. 9, column 8, in the same line as SEQ ID NO. 120241, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 120241 is depicted, plastidic is mentioned, or a mitochondrial transit peptide in case in Table I, application no. 9, in column 6 in the same line as SEQ ID NO. 120241 is depicted, mitochondrial is mentioned; which is operable linked, too.

In another embodiment thereof the expression cassette does not comprise an additional transit peptide in case in Table I, application no. 9, in column 6 in the same line as SEQ ID NO. 120241 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 90165, or a homolog or fragment thereof, or a homolog thereof being depicted in Table I, application no. 9, column 8, in the same line as SEQ ID NO. 90165, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 90165 is depicted, plastidic is mentioned, or a mitochondrial transit peptide in case in Table I, application no. 9, in column 6 in the same line as SEQ ID NO. 90165 is depicted, mitochondrial is mentioned; which is operable linked, too.

In another embodiment thereof the expression cassette does not comprise an additional transit peptide in case in Table I, application no. 9, in column 6 in the same line as SEQ ID NO. 90165 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 120309, or a homolog or fragment thereof, or a homolog thereof being depicted in Table I, application no. 9, column 8, in the same line as SEQ ID NO. 120309, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 120309 is depicted, plastidic is mentioned, or a mitochondrial transit peptide in case in Table I, application no. 9, in column 6 in the same line as SEQ ID NO. 120309 is depicted, mitochondrial is mentioned; which is operable linked, too.

In another embodiment thereof the expression cassette does not comprise an additional transit peptide in case in Table I, application no. 9, in column 6 in the same line as SEQ ID NO. 120309 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 120436, or a homolog or fragment thereof, or a homolog thereof being depicted in Table I, application no. 9, column 8, in the same line as SEQ ID NO. 120436, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 120436 is depicted, plastidic is mentioned, or a mitochondrial transit peptide in case in Table I, application no. 9, in column 6 in the same line as SEQ ID NO. 120436 is depicted, mitochondrial is mentioned; which is operable linked, too.

In another embodiment thereof the expression cassette does not comprise an additional transit peptide in case in Table I, application no. 9, in column 6 in the same line as SEQ ID NO. 120436 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 64964, or a homolog or fragment thereof, or a homolog thereof being depicted in Table I, application no. 9, column 8, in the same line as SEQ ID NO. 64964, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 64964 is depicted, plastidic is mentioned, or a mitochondrial transit peptide in case in Table I, application no. 9, in column 6 in the same line as SEQ ID NO. 64964 is depicted, mitochondrial is mentioned; which is operable linked, too.

In another embodiment thereof the expression cassette does not comprise an additional transit peptide in case in Table I, application no. 9, in column 6 in the same line as SEQ ID NO. 64964 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 91958, or a homolog or fragment thereof, or a homolog thereof being depicted in Table I, application no. 9, column 8, in the same line as SEQ ID NO. 91958, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 91958 is depicted, plastidic is mentioned, or a mitochondrial transit peptide in case in Table I, application no. 9, in column 6 in the same line as SEQ ID NO. 91958 is depicted, mitochondrial is mentioned; which is operable linked, too.

In another embodiment thereof the expression cassette does not comprise an additional transit peptide in case in Table I, application no. 9, in column 6 in the same line as SEQ ID NO. 91958 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 117495, or a homolog or fragment thereof, or a homolog thereof being depicted in Table I, application no. 9, column 8, in the same line as SEQ ID NO. 117495, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 117495 is depicted, plastidic is mentioned, or a mitochondrial transit peptide in case in Table I, application no. 9, in column 6 in the same line as SEQ ID NO. 117495 is depicted, mitochondrial is mentioned; which is operable linked, too.

In another embodiment thereof the expression cassette does not comprise an additional transit peptide in case in Table I, application no. 9, in column 6 in the same line as SEQ ID NO. 117495 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 118212, or a homolog or fragment thereof, or a homolog thereof being depicted in Table I, application no. 9, column 8, in the same line as SEQ ID NO. 118212, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 118212 is depicted, plastidic is mentioned, or a mitochondrial transit peptide in case in Table I, application no. 9, in column 6 in the same line as SEQ ID NO. 118212 is depicted, mitochondrial is mentioned; which is operable linked, too.

In another embodiment thereof the expression cassette does not comprise an additional transit peptide in case in Table I, application no. 9, in column 6 in the same line as SEQ ID NO. 118212 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 16883, or a homolog or fragment thereof, or a homolog thereof being depicted in Table I, application no. 9, column 8, in the same line as SEQ ID NO. 16883, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 9, in column 6 in the same line as SEQ ID NO. 16883 is depicted, plastidic is mentioned, or a mitochondrial transit peptide in case in Table I, application no. 9, in column 6 in the same line as SEQ ID NO. 16883 is depicted, mitochondrial is mentioned; which is operable linked, too.

In another embodiment thereof the expression cassette does not comprise an additional transit peptide in case in Table I, application no. 9, in column 6 in the same line as SEQ ID NO. 16883 is depicted, non-targeted is mentioned.

[0165.1.8.9] to [0170.1.8.9] for the disclosure of these paragraphs see [0165.1.8.8] to [0170.1.8.8] above.

The invention further provides a vector, comprising a nucleic acid molecule comprising 1) a nucleic acid molecule selected from the group consisting of:
(a) a nucleic acid molecule encoding a polypeptide shown in Table II, column 5 or 8, application no. 9, preferably shown in Table II A, application no. 9, in column 5, or in Table II A, application no. 9, column 8, or in Table II B, application no. 9, column 8, or a homolog or a fragment thereof;
(b) a nucleic acid molecule shown in Table I, application no. 9, column 5 or 8, preferably shown in Table I A, application no. 9, in column 5, or in Table I A, application no. 9, column 8, or in Table I B, application no. 9, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 9, preferably shown in Table II A, application no. 9, in column 5, or in Table II A, application no. 9, column 8, or in Table II B, application no. 9, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 9, preferably shown in Table I A, application no. 9, in column 5, or in Table I A, application no. 9, column 8, or in Table I B, application no. 9, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 9;
(i) a nucleic acid molecule encoding a polypeptide having the activity represented by a protein as depicted in column 5 of Table II, application no. 9;
(j) a nucleic acid molecule comprising a polynucleotide which is obtained by amplifying a cDNA library or a genomic library using the primers in Table III, application no. 9, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 9.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of

Table II, application no. 9, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 9.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 9, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 9, in the same line as of the respective nucleic acid molecule according to 1) “plastidic” is depicted, or encoding a mitochondrial transit peptide, especially in case in column 6 of Table I, application no. 9, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 9, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 9, in the same line as of the respective nucleic acid molecule according to 1) “plastidic” is depicted, or encoding a mitochondrial transit peptide, especially in case in column 6 of Table I, application no. 9, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operable linked; a nucleic acid molecule encoding a transit peptide, especially in case in column 6 of Table I, application no. 9, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 9, in the same line as of the respective nucleic acid molecule according to 1) “plastidic” is depicted, or encoding a mitochondrial transit peptide, especially in case in column 6 of Table I, application no. 9, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 9 (in the same line).

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 9, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 9.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 9, in the same line as of the respective nucleic acid molecule according to 1) “plastidic” is depicted, or encoding a mitochondrial transit peptide, especially in case in column 6 of Table I, application no. 9, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 9, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 9, in the same line as of the respective nucleic acid molecule according to 1) “plastidic” is depicted, or encoding a mitochondrial transit peptide, especially in case in column 6 of Table I, application no. 9, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 9 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 9, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 9 (in the same line) and confers a generation or an increase of the respective fine chemical in a non-human organism or a part thereof as compared to a corresponding wild-type non-human organism or part thereof.

In a preferred embodiment of said vector said nucleic acid molecule according to 1 (a), (b), (c), (d), (e), (f), (g), (h), (i), (j) or (k), said promoter and optionally said nucleic acid molecule encoding a transit peptide are encompassed in an expression cassette. Accordingly in an embodiment the invention provides a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

The invention further provides a vector comprising a nucleic acid molecule comprising 1) a nucleic acid molecule selected from the group consisting of:
(a) a nucleic acid molecule encoding a polypeptide shown in Table II, column 5 or 8, application no. 9, preferably shown in Table II A, application no. 9, in column 5, or in Table II A, application no. 9, column 8, or in Table II B, application no. 9, column 8, or a homolog or a fragment thereof;
(b) a nucleic acid molecule shown in Table I, application no. 9, column 5 or 8, preferably shown in Table I A, application no. 9, in column 5, or in Table I A, application no. 9, column 8, or in Table I B, application no. 9, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 9, preferably shown in Table II A, application no. 9, in column 5, or in Table II A, application no. 9, column 8, or in Table II B, application no. 9, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 9, preferably shown in Table I A, application no. 9, in column 5, or in Table I A, application no. 9, column 8, or in Table I B, application no. 9, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 9;
(i) a nucleic acid molecule encoding a polypeptide having the activity represented by a protein as depicted in column 5 of Table II, application no. 9;
(j) a nucleic acid molecule comprising a polynucleotide which is obtained by amplifying a cDNA library or a genomic library using the primers in Table III, application no. 9, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto;
   whereby, preferably, the nucleic acid molecule according to 1 (a), (b), (c), (d), (e), (f), (g), (h), (i), (j) or (k) distinguishes over the sequence depicted in Table I A and/or I B, application no. 9, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 9, column 5 or 8, or the coding region thereof, respectively. In another embodiment, the nucleic acid molecule of the present invention, preferably the coding region thereof, is 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 9, column 5 or 8, or the coding region thereof, respectively. In a further embodiment the nucleic acid molecule does not encode the polypeptide sequence shown in Table II A and/or II B, application no. 9, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 9, column 5 or 8 and does not encode a protein of the sequence shown in Table II A and/or II B, application no. 9, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 9, column 5 and 8, or the coding region thereof. In a further embodiment, the protein of the present invention is at least 30%,40%, 50%, 60%, 70%, 80%, 90%, 95% identical to protein sequence depicted in Table II A and/or II B, application no. 9, column 5 or 8 but less than 100%, preferably less than 99.999%, 99.99% or 99.9%, more preferably less than 99%, 985, 97%, 96% or 95% identical to the sequence shown in Table II A and/or II B, application no. 9, column 5 or 8.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 9.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of

Table II, application no. 9, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 9.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 9, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 9, in the same line as of the respective nucleic acid molecule according to 1) “plastidic” is depicted, or encoding a mitochondrial transit peptide, especially in case in column 6 of Table I, application no. 9, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 9, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 9, in the same line as of the respective nucleic acid molecule according to 1) “plastidic” is depicted, or encoding a mitochondrial transit peptide, especially in case in column 6 of Table I, application no. 9, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 9, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 9, in the same line as of the respective nucleic acid molecule according to 1) “plastidic” is depicted, or encoding a mitochondrial transit peptide, especially in case in column 6 of Table I, application no. 9, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 9.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 9, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 9.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 9, in the same line as of the respective nucleic acid molecule according to 1) “plastidic” is depicted, or encoding a mitochondrial transit peptide, especially in case in column 6 of Table I, application no. 9, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 9, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 9, in the same line as of the respective nucleic acid molecule according to 1) “plastidic” is depicted, or encoding a mitochondrial transit peptide, especially in case in column 6 of Table I, application no. 9, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 9 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 9, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 9 and confers a generation or an increase of the respective fine chemical in a non-human organism or a part thereof as compared to a corresponding wild-type non-human organism or part thereof.

[0181.1.8.9] to [0209.1.8.9] for the disclosure of these paragraphs see [0181.1.8.8] to [0209.1.8.8] above.

The genes of the invention, coding for an activity selected from the group consisting of 3-oxoacyl-[acyl-carrier-protein] reductase, 3-oxoacyl-[acyl-carrier-protein] synthase, AAS bifunctional protein, acid phosphatase precursor, acid shock protein, acyl transferase, acylCoA synthase, arginine decarboxylase, aromatic acid decarboxylase, asparaginase, At2g45420-protein, ATPase epsilon subunit, b0195-protein, b1234-protein, b1248-protein, b1280-protein, b1330-protein, b1470-protein, b1670-protein, b1672-protein, b1837-protein, b2032-protein, b2107-protein, b2399-protein, b2474-protein, b2613-protein, b2682-protein, b3442-protein, b3534-protein, b4023-protein, bifunctional purine biosynthesis protein, branched chain amino acid ABC transporter ATP-binding protein, branched-chain amino acid permease, carbamoyl-phosphate synthase subunit, carbon dioxide concentrating mechanism protein, cation-transporting ATPase, cell division control protein, coproporphyrinogen III oxidase, CTP synthase, CTP synthetase, dihydroxyacetone kinase, DnaJ-like chaperone, electron transport complex protein, ethanolamine utilization protein, F-box protein, flavodoxin, fumarate hydratase, glucose-6-phosphate 1-dehydrogenase, glutaredoxin, glycerol-3-phosphate dehydrogenase, heat shock transcription factor, HesB/YadR/YfhF family protein, isopropylmalate isomerase large subunit, metal-dependent hydrolase, molecular chaperone, monothiol glutaredoxin, multidrug resistance protein, oxidoreductase, permease protein of ABC transporter, phosphate import ATP-binding protein, phosphoadenosine phosphosulfate reductase , phosphofructokinase, phosphotriesterase, protein phosphatase, pyruvate kinase, recombinase A, sec-independent protein translocase, sll1032-protein, ThiF family protein, threonine dehydratase, threonine efflux protein, transcription factor, TTC1386-protein, ydr339c-protein, ydr507c-protein, and ymr013c-protein are also called “FCRP genes”.

[0211.1.8.9] to [0225.1.8.9] for the disclosure of these paragraphs see [0211.1.8.8] to [0225.1.8.8] above.

In addition to the sequence mentioned in Table I, application no. 9, column 5 or 8, preferably the coding region thereof, or its homologs or fragments, it may be advantageous additionally to express and/or mutate further genes in the non-human organisms. Especially advantageously, additionally at least one further gene of the biosynthetic pathway of the fine chemical is expressed in the non-human organisms such as plants or microorganisms. It is also possible that the regulation of the natural genes has been modified advantageously so that the gene and/or its gene product is no longer subject to the regulatory mechanisms which exist in the non-human organisms. This leads to an increased synthesis of the respective fatty acid since, for example, feedback regulations no longer exist to the same extent or not at all. In addition it might be advantageously to combine the nucleic acids sequences of the invention containing the sequences shown in the respective line in Table I, application no. 9, column 5 or 8, preferably the coding region thereof, or homologs or fragments thereof, with genes which generally support or enhance the growth or yield of the target non-human organisms, for example genes which lead to faster growth rate of non human organism like microorganisms or plants or genes which produce stress-, pathogen-, or herbicide-resistant plants.

[0227.1.8.9] to [0239.1.8.9] for the disclosure of these paragraphs see [0227.1.8.8] to [0239.1.8.8] above.

In an embodiment of the present invention the nucleic acid construct of the present invention comprises a nucleic acid molecule as depicted in the respective line in Table I, application no. 9, column 5 or 8, preferably the coding region thereof, a fragment or a homolog thereof, and a plant promoter, such as the Big 35S promoter, the DC3 promoter, the PC

Ubi promoter, the LegB4-promoter, the Super promoter, the USP promoter or the like. In another embodiment the nucleic acid construct of the present invention comprises a nucleic acid molecule as depicted in the respective line in Table I, application no. 9, column 5 or 8, preferably the coding region thereof, a fragment or a homolog thereof, and a plant promoter such as, for example, the USP, the LegB4-, the DC3 promoter or the ubiquitin promoter from parsley or other herein mentioned promoters. In a further embodiment the nucleic acid construct of the present invention comprises a nucleic acid molecule as depicted in the respective line in Table I, application no. 9, column 5 or 8, preferably the coding region thereof, a fragment or a homolog thereof, and a plant promoter, such as the Big 35S promoter, the PC Ubi promoter, the Super promoter, the USP promoter or the like. In addition different terminators may advantageously be used in these nucleic acid constructs; examples for transcriptional termination are polyadenylation signals.

[0241.1.8.9] to [0245.1.8.9] for the disclosure of these paragraphs see [0241.1.8.8] to [0245.1.8.8] above.

Other preferred sequences for use in operable linkage in gene expression constructs are targeting sequences, which are required for targeting the gene product into specific cell compartments (for a review, see Kermode, Crit. Rev. Plant Sci. 15 (4), 285 (1996) and references cited therein), for example into the vacuole, the nucleus, all types of plastids, such as amyloplasts, chloroplasts, chromoplasts, the extracellular space, the mitochondria, the endoplasmic reticulum, elaioplasts, peroxisomes, glycosomes, and other compartments of cells or extracellular; preferred are sequences, which are involved in targeting to plastids as mentioned above. Sequences, which must be mentioned in this context are, in particular, the signalpeptideor transit-peptide-encoding sequences which are known per se. For example, plastidtransit-peptide-encoding sequences enable the targeting of the expression product into the plastids of a plant cell. Targeting sequences are also known for eukaryotic and to a lower extent for prokaryotic organisms and can advantageously be operable linked with the nucleic acid molecule of the present invention as shown in Table I, application no. 9, columns 5 and 8, preferably the coding region thereof, or homologs or fragments thereof, and described herein to achieve an expression in one of said compartments or extracellular.

[0247.1.8.9] to [0266.1.8.9] for the disclosure of these paragraphs see [0247.1.8.8] to [0266.1.8.8] above.

An embodiment of the invention relates to a vector where the nucleic acid molecule according to the invention is linked operably to regulatory sequences which permit the expression in a prokaryotic or eukaryotic host. A further embodiment of the invention relates to a vector in which a nucleic acid sequence encoding one of the polypeptides shown in the respective line in Table II, application no. 9, columns 5 or 8, or homologs or fragments thereof, is functionally linked to a targeting sequence such as a plastidial or mitochondrial targeting sequence. A further embodiment of the invention relates to a vector in which a nucleic acid sequence encoding one of the polypeptides shown in the respective line in Table II, application no. 9, columns 5 or 8, or homologs or fragments thereof, is functionally linked to (a) regulatory sequence which permit the expression in an organelle, such as plastids or mitochondria, preferably plastids.

A further embodiment of the invention relates to a vector which comprises a nucleic acid sequence encoding one of the polypeptides shown in the respective line in Table II, application no. 9, columns 5 or 8, or homologs or fragments thereof, but no additional targeting sequence.

[0268.1.8.9] to [0273.1.8.9] for the disclosure of these paragraphs see [0268.1.8.8] to [0273.1.8.8] above.

A further object of the invention relates to the use of a nucleic acid construct, e.g. an expression cassette, comprising one or more nucleic acid molecules encoding one or more polypeptides as depicted in the respective line(s) in Table II, application no. 9, columns 5 or 8, or homologs or fragments thereof, or comprising one or more nucleic acid molecules as depicted in the respective line(s) in Table I, application no. 9, columns 5 or 8, preferably the coding region thereof, or homologs or fragments thereof, or comprising a nucleic acid molecule hybridizing therewith for the transformation of a microorganism or a plant cell, plant tissues or a part of a plant.

In doing so, depending on the choice of promoter, the nucleic acid molecules shown in the respective line in Table I, application no. 9, column 5 or 8, preferably the coding region thereof, or homologs or fragments thereof, or the nucleic acid molecules encoding a polypeptide as depicted in the respective line in Table II, application no. 9, column 5 or 8, or homologs or fragments thereof, can be expressed in the whole plant or specifically e.g. in the leaves, in the seeds, the nodules, in roots, in the stem or other parts of the plant. Those transgenic plants overproducing sequences, e.g. as depicted in the respective line in Table I, application no. 9, column 5 or 8, preferably the coding region thereof, or homologs or fragments thereof, the plant cell, plant tissues, plants, the reproductive material thereof, or parts thereof are a further object of the present invention.

Within the framework of the present invention, the production of the linolenic acid is due to the generation or over-expression of one or more polypeptides as depicted in the respective line(s) in Table II, application no. 9, column 5 or 8, or homologs or fragments thereof, or encoded by the corresponding nucleic acid molecules as depicted in the respective line(s) in Table I, application no. 9, column 5 or 8, preferably the coding region thereof, or homologs or fragments thereof, in the non-human organism according to the invention, advantageously in the transgenic microorganism or plant according to the invention, at least for the duration of at least one plant generation.

A constitutive expression of the polypeptide as depicted in the respective line in Table II, application no. 9, column 5 or 8, or homologs or fragments thereof, or encoded by the respective nucleic acid molecule as depicted in the respective line in Table I, application no. 9, column 5 or 8, preferably the coding region thereof, or homologs or fragments thereof, may be advantageous. On the other hand, an inducible expression may also appear desirable. Expression of the polypeptide sequences of the invention can be either directed to the cytosol or to the organelles, such as plastids or mitochondria, preferably the plastids of the host cells, preferably the plant cells, or non-targeted.

for the disclosure of this paragraph see [0278.1.8.8] above.

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 9, column 5 or 8, preferably shown in Table II A, application no. 9, column 5, or in Table II A, application no. 9, column 8, or in Table II B, application no. 9, column 8, or a homolog or a fragment thereof;
   (b) a nucleic acid molecule shown in Table I, application no. 9, column 5 or 8, preferably shown in Table I A, application no. 9, column 5, or in Table I A, application no. 9, column 8, or in Table I B, application no. 9, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 9, preferably shown in Table II A, application no. 9, column 5, or in Table II A, application no. 9, column 8, or in Table II B, application no. 9, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 9, preferably shown in Table I A, application no. 9, column 5, or in Table I A, application no. 9, column 8, or in Table I B, application no. 9, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 9;
   (i) a nucleic acid molecule encoding a polypeptide having the activity represented by a respective protein as depicted in column 5 of Table II, application no. 9;
   (j) a nucleic acid molecule comprising a polynucleotide which is obtained by amplifying a cDNA library or a genomic library using the primers in Table III, application no. 9, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising an expression cassette comprising
[0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 9, column 5 or 8, preferably shown in Table II A, application no. 9, column 5, or in Table II A, application no. 9, column 8, or in Table II B, application no. 9, column 8, or a homolog or a fragment thereof;
   (b) a nucleic acid molecule shown in Table I, application no. 9, column 5 or 8, preferably shown in Table I A, application no. 9, column 5, or in Table I A, application no. 9, column 8, or in Table I B, application no. 9, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 9, preferably shown in Table II A, application no. 9, column 5, or in Table II A, application no. 9, column 8, or in Table II B, application no. 9, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 9, preferably shown in Table I A, application no. 9, column 5, or in Table I A, application no. 9, column 8, or in Table I B, application no. 9, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 9;
   (i) a nucleic acid molecule encoding a polypeptide having the activity represented by a respective protein as depicted in column 5 of Table II, application no. 9;
   (j) a nucleic acid molecule comprising a polynucleotide which is obtained by amplifying a cDNA library or a genomic library using the primers in Table III, application no. 9, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide; which are operable linked;]
   or comprising or being transformed by a vector comprising
[1) a nucleic acid molecule selected from the group consisting of
   (a) a nucleic acid molecule encoding a polypeptide shown in Table II, application no. 9, column 5 or 8, preferably shown in Table II A, application no. 9, column 5, or in Table II A, application no. 9, column 8, or in Table II B, application no. 9, column 8, or a homolog or a fragment thereof;
   (b) a nucleic acid molecule shown in Table I, application no. 9, column 5 or 8, preferably shown in Table I A, application no. 9, column 5, or in Table I A, application no. 9, column 8, or in Table I B, application no. 9, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 9, preferably shown in Table II A, application no. 9, column 5, or in Table II A, application no. 9, column 8, or in Table II B, application no. 9, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 9, preferably shown in Table I A, application no. 9, column 5, or in Table I A, application no. 9, column 8, or in Table I B, application no. 9, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 9;
   (i) a nucleic acid molecule encoding a polypeptide having the activity represented by a respective protein as depicted in column 5 of Table II, application no. 9;
   (j) a nucleic acid molecule comprising a polynucleotide which is obtained by amplifying a cDNA library or a genomic library using the primers in Table III, application no. 9, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 9.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 9 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 9, in the same line as of the respective nucleic acid molecule according to 1) “plastidic” is depicted, or encoding a mitochondrial transit peptide, especially in case in column 6 of Table I, application no. 9, in the same line as of the respective nucleic acid molecule according to 1) “mitochondrial” is depicted.

In another embodiment of said transgenic non-human organism the expression cassette does not comprise 2) a nucleic acid molecule encoding a transit peptide, especially in case in column 6 of Table I, application no. 9, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 9.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 9, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 9, in the same line as of the respective nucleic acid molecule according to 1) “plastidic” is depicted, or encoding a mitochondrial transit peptide, especially in case in column 6 of Table I, application no. 9, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 9, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 9, in the same line as of the respective nucleic acid molecule according to 1) “plastidic” is depicted, or encoding a mitochondrial transit peptide, especially in case in column 6 of Table I, application no. 9, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 9, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 9, in the same line as of the respective nucleic acid molecule according to 1) “plastidic” is depicted, or encoding a mitochondrial transit peptide, especially in case in column 6 of Table I, application no. 9, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 9.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 9, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 9.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 9, in the same line as of the respective nucleic acid molecule according to 1) “plastidic” is depicted, or encoding a mitochondrial transit peptide, especially in case in column 6 of Table I, application no. 9, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 9, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 9, in the same line as of the respective nucleic acid molecule according to 1) “plastidic” is depicted, or encoding a mitochondrial transit peptide, especially in case in column 6 of Table I, application no. 9, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 9 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 9, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 9 (in the same line) and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 9, column 5 or 8, preferably shown in Table II A, application no. 9, column 5, or in Table II A, application no. 9, column 8, or in Table II B, application no. 9, column 8, or a homolog or a fragment thereof;
   (b) a nucleic acid molecule shown in Table I, application no. 9, column 5 or 8, preferably shown in Table I A, application no. 9, column 5, or in Table I A, application no. 9, column 8, or in Table I B, application no. 9, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 9, preferably shown in Table II A, application no. 9, column 5, or in Table II A, application no. 9, column 8, or in Table II B, application no. 9, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 9, preferably shown in Table I A, application no. 9, column 5, or in Table I A, application no. 9, column 8, or in Table I B, application no. 9, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 9;
   (i) a nucleic acid molecule encoding a polypeptide having the activity represented by a respective protein as depicted in column 5 of Table II, application no. 9;
   (j) a nucleic acid molecule comprising a polynucleotide which is obtained by amplifying a cDNA library or a genomic library using the primers in Table III, application no. 9, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising an expression cassette comprising
[0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 9, column 5 or 8, preferably shown in Table II A, application no. 9, column 5, or in Table II A, application no. 9, column 8, or in Table II B, application no. 9, column 8, or a homolog or a fragment thereof;
   (b) a nucleic acid molecule shown in Table I, application no. 9, column 5 or 8, preferably shown in Table I A, application no. 9, column 5, or in Table I A, application no. 9, column 8, or in Table I B, application no. 9, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 9, preferably shown in Table II A, application no. 9, column 5, or in Table II A, application no. 9, column 8, or in Table II B, application no. 9, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 9, preferably shown in Table I A, application no. 9, column 5, or in Table I A, application no. 9, column 8, or in Table I B, application no. 9, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 9;
   (i) a nucleic acid molecule encoding a polypeptide having the activity represented by a respective protein as depicted in column 5 of Table II, application no. 9;
   (j) a nucleic acid molecule comprising a polynucleotide which is obtained by amplifying a cDNA library or a genomic library using the primers in Table III, application no. 9, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      and, if desired
2) nucleic acid molecule encoding a transit peptide, preferably encoding a plastidal transit peptide or a mitochondrial transit peptide;
   which are operable linked;]
   or comprising or being transformed by a vector comprising
[1) a nucleic acid molecule selected from the group consisting of
   (a) a nucleic acid molecule encoding a polypeptide shown in Table II, application no. 9, column 5 or 8, preferably shown in Table II A, application no. 9, column 5, or in Table II A, application no. 9, column 8, or in Table II B, application no. 9, column 8, or a homolog or a fragment thereof;
   (b) a nucleic acid molecule shown in Table I, application no. 9, column 5 or 8, preferably shown in Table I A, application no. 9, column 5, or in Table I A, application no. 9, column 8, or in Table I B, application no. 9, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 9, preferably shown in Table II A, application no. 9, column 5, or in Table II A, application no. 9, column 8, or in Table II B, application no. 9, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 9, preferably shown in Table I A, application no. 9, column 5, or in Table I A, application no. 9, column 8, or in Table I B, application no. 9, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 9;
   (i) a nucleic acid molecule encoding a polypeptide having the activity represented by a respective protein as depicted in column 5 of Table II, application no. 9;
   (j) a nucleic acid molecule comprising a polynucleotide which is obtained by amplifying a cDNA library or a genomic library using the primers in Table III, application no. 9, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      whereby, preferably, the nucleic acid molecule according to 1 (a), (b), (c), (d), (e), (f), (g), (h), (i), (j) or (k) distinguishes over the sequence depicted in Table I A and/or I B, application no. 9, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or

I B, application no. 9, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 9, column 5 or 8, or the coding region thereof, respectively. In a further embodiment the nucleic acid molecule does not encode the polypeptide sequence shown in Table II A and/or II B, application no. 9, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 9, column 5 or 8 and does not encode a protein of the sequence shown in Table II A and/or II B, application no. 9, column 5 or 8. Accordingly, in one embodiment, the protein encoded by a sequence of a nucleic acid according to 1 (a), (b), (c), (d), (e), (f), (g), (h), (i), (j) or (k) does not consist of the sequence shown in Table I A and/or I B, application no. 9, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 9, column 5 or 8 but less than 100%, preferably less than 99.999%, 99.99%, 99.9%, 99%, 98%, 97%, 96% or 95% identical to the sequence shown in Table II A and/or II B, application no. 9, column 5 or 8;

as well as respective transgenic cells, tissue, parts of such non-human organism, e.g. plant cells, plant tissue, part of plants, like leaves, roots, stems, blossoms, seeds, fruits, pollen and the like.
(All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 9.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 9 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 9, in the same line as of the respective nucleic acid molecule according to 1) “plastidic” is depicted, or encoding a mitochondrial transit peptide, especially in case in column 6 of Table I, application no. 9, in the same line as of the respective nucleic acid molecule according to 1) “mitochondrial” is depicted.

In another embodiment thereof the expression cassette does not comprise 2) a nucleic acid molecule encoding a transit peptide, especially in case in column 6 of Table I, application no. 9, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 9.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the same line in column 5 of Table II, application no. 9, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 9, in the same line as of the respective nucleic acid molecule according to 1) “plastidic” is depicted, or encoding a mitochondrial transit peptide, especially in case in column 6 of Table I, application no. 9, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 9, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 9, in the same line as of the respective nucleic acid molecule according to 1) “plastidic” is depicted, or encoding a mitochondrial transit peptide, especially in case in column 6 of Table I, application no. 9, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 9, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 9, in the same line as of the respective nucleic acid molecule according to 1) “plastidic” is depicted, or encoding a mitochondrial transit peptide, especially in case in column 6 of Table I, application no. 9, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 9.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 9, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 9.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 9, in the same line as of the respective nucleic acid molecule according to 1) “plastidic” is depicted, or encoding a mitochondrial transit peptide, especially in case in column 6 of Table I, application no. 9, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 9, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 9, in the same line as of the respective nucleic acid molecule according to 1) “plastidic” is depicted, or encoding a mitochondrial transit peptide, especially in case in column 6 of Table I, application no. 9, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 9 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 9, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 9 and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

[0291.1.8.9] to [0299.1.8.9] for the disclosure of these paragraphs see [0291.1.8.8] to [0299.1.8.8] above.

The term “biological active portion” or “biological activity” means a polypeptide as depicted in the respective line in Table II, application no. 9, column 3 or a portion of said polypeptide which still has at least 10% or 20%, preferably 30%, 40%, 50% or 60%, especially preferably 70%, 75%, 80%, 90% or 95% of the enzymatic or biological activity of the natural or starting enzyme or protein.

[0301.1.8.9] to [0304.1.8.9] for the disclosure of these paragraphs see [0301.1.8.8] to [0304.1.8.8] above.

Synthetic oligonucleotide primers for the amplification, e.g. as shown in the respective line in Table III, application no. 9, column 8, by means of polymerase chain reaction can be generated on the basis of a sequence shown herein, for example the sequence shown in the respective line in Table I, application no. 9, columns 5 or 8, or the sequences derived from Table II, application no. 9, columns 5 or 8, respectively.

Moreover, it is possible to identify a conserved region of a protein from various organism by carrying out protein sequence alignments with the polypeptide encoded by the nucleic acid molecules of the present invention, in particular with the sequences encoded by the nucleic acid molecule shown in the respective line in column 5 or 8 of Table I, application no. 9, from which conserved regions, and in turn, degenerate primers can be derived. Conserved regions are those, which show a very little variation in the amino acid in one particular position of several homologs from different origin. The consensus sequence and polypeptide motifs shown in the respective line in column 8 of Table IV, application no. 9, are derived from such alignments of homologous proteins.

In an embodiment of the present invention, in the method of the present invention the activity of a polypeptide comprising or consisting of a consensus sequence or at least one polypeptide motif shown in the respective line in Table IV, application no. 9, column 8, is increased. In another embodiment thereof, the method of the present invention relates to a polypeptide comprising or consisting of a consensus sequence shown in the respective line in Table IV, application no. 9, column 8 or comprising at least one polypeptide motif shown in the respective line in Table IV, application no. 9, column 8, whereby not more than 20, 15, 10, 9, 8, 7, 6, 5 4, 3, 2 or 1, or 0 of the amino acid positions indicated can be replaced by any amino acid. In another embodiment thereof, the method of the present invention relates to a polypeptide comprising or consisting of a consensus sequence shown in the respective line in Table IV, application no. 9, column 8 or comprising at least one polypeptide motif shown in the respective line in Table IV, application no. 9, column 8, whereby not more than 15%, 10%, 5%, 4%, 3%, 2% or 1%, or 0% of the amino acid positions defined as distinct specific amino acids are/is replaced by another amino acid. In another embodiment thereof, the method of the present invention relates to a polypeptide comprising or consisting of a consensus sequence shown in the respective line in Table IV, application no. 9, column 8 or comprising at least one polypeptide motif shown in the respective line in Table IV, application no. 9, column 8, whereby not more than 20, 15, 10, 9, 8, 7, 6, 5, 4, 3, 2 or 1, or 0 amino acids are inserted into a consensus sequence or polypeptide motif.

In an embodiment of the present invention, in the method of the present invention the activity of a polypeptide comprising or consisting of a consensus sequence or at least one polypeptide motif shown in the respective line in Table IV, application no. 9, column 8, is increased, whereby said polypeptide distinguishes over the sequence depicted in Table II, application no. 9, columns 5 or 8 by one or more amino acids.

In one embodiment, said polypeptide of the invention distinguishes over the sequence shown in the respective line in Table II, application no. 9, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II B, application no. 9, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in Table II, application no. 9, columns 5 or 8.

[0309.1.8.9] to [0321.1.8.9] for the disclosure of these paragraphs see [0309.1.8.8] to [0321.1.8.8] above.

Polypeptides having above-mentioned activity, i.e. conferring the production or the increased production of the fine chemical linolenic acid as compared to a corresponding, e.g. non-transformed, wild type non-human organism, like a microorganism or a plant cell, plant or part thereof, can be encoded by other DNA sequences which hybridize to the sequences shown in the respective line in Table I, application no. 9, columns 5 and 8, preferably the coding region thereof, at least under relaxed hybridization conditions and which encode the expression of polypeptides conferring the production or the increased production of the respective fine chemical linolenic acid as compared to a corresponding, e.g. non-transformed, wild type non-human organism, like a microorganism or a plant cell, plant or part thereof.

[0323.1.8.9] to [0329.1.8.9] for the disclosure of these paragraphs see [0323.1.8.8] to [0329.1.8.8] above.

Further, the nucleic acid molecule of the invention comprises a nucleic acid molecule, which is a complement of one of the nucleotide sequences of above-mentioned nucleic acid molecules or a portion thereof. A nucleic acid molecule or its sequence which is complementary to one of the nucleotide molecules or sequences shown in the respective line in Table I, application no. 9, columns 5 or 8, preferably the coding region thereof, homologs or fragments thereof, is one which is sufficiently complementary to one of the nucleotide molecules or sequences shown in the respective line in Table I, application no. 9, columns 5 or 8, preferably the coding region thereof, homologs or fragments thereof, such that it can hybridize to one of the nucleotide sequences shown in Table I, application no. 9, columns 5 or 8, preferably the coding region thereof, homologs or fragments thereof, thereby forming a stable duplex. Preferably, the hybridization is performed under stringent hybridization conditions. However, a complement of one of the herein disclosed sequences is preferably a sequence complement thereto according to the base pairing of nucleic acid molecules well known to the skilled person. For example, the bases A and G undergo base pairing with the bases T and U or C, resp. and visa versa. Modifications of the bases can influence the base-pairing partner.

The nucleic acid molecule of the invention comprises a nucleotide sequence which is at least about 30%, 35%, 40%, 45%, 50%, 55%, 60%, 65%, 70%, 75%, 80%, 90%, 95%, 96%, 97%, 98%, 99%, 99.5% or more homologous to a nucleotide sequence shown in Table I, application no. 9, columns 5 or 8, preferably the coding region thereof, homologs or fragments thereof, and preferably has above-mentioned activity, in particular conferring the production or the increased production of the fine chemical linolenic acid, respectively, after increasing the activity or an activity of a gene as shown in the respective line in Table I or of a gene product, e.g. as shown in the respective line in Table II, application no. 9, column 5 or 8, by for example in one embodiment expression either in the cytosol or in an organelle such as a plastid or mitochondria or both, preferably in a plastid, or in another embodiment non-targeted or targeted.

The nucleic acid molecule of the invention comprises a nucleotide sequence or molecule which hybridizes, preferably hybridizes under stringent conditions as defined herein, to one of the nucleic acid molecule shown in the respective line in Table I, preferably Table IB, application no. 9, columns 5 or 8, preferably the coding region thereof, fragments or homologs thereof, and encodes a protein having above-mentioned activity, e.g. conferring the production or the increased production of the respective fine chemical linolenic acid as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted, and optionally, the activity thereof is selected from the group consisting of 3-oxoacyl-[acyl-carrier-protein] reductase, 3-oxoacyl-[acyl-carrier-protein] synthase, AAS bifunctional protein, acid phosphatase precursor, acid shock protein, acyl transferase, acyl-CoA synthase, arginine decarboxylase, aromatic acid decarboxylase, asparaginase, At2g45420-protein, ATPase epsilon subunit, b0195-protein, b1234-protein, b1248-protein, b1280-protein, b1330-protein, b1470-protein, b1670-protein, b1672-protein, b1837-protein, b2032-protein, b2107-protein, b2399-protein, b2474-protein, b2613-protein, b2682-protein, b3442-protein, b3534-protein, b4023-protein, bifunctional purine biosynthesis protein, branched chain amino acid ABC transporter ATP-binding protein, branched-chain amino acid permease, carbamoyl-phosphate synthase subunit, carbon dioxide concentrating mechanism protein, cation-transporting ATPase, cell division control protein, coproporphyrinogen III oxidase, CTP synthase, CTP synthetase, dihydroxyacetone kinase, DnaJ-like chaperone, electron transport complex protein, ethanolamine utilization protein, F-box protein, flavodoxin, fumarate hydratase, glucose-6-phosphate 1-dehydrogenase, glutaredoxin, glycerol-3-phosphate dehydrogenase, heat shock transcription factor, HesB/YadR/YfhF family protein, isopropylmalate isomerase large subunit, metal-dependent hydrolase, molecular chaperone, monothiol glutaredoxin, multidrug resistance protein, oxidoreductase, permease protein of ABC transporter, phosphate import ATP-binding protein, phosphoadenosine phosphosulfate reductase, phosphofructokinase, phosphotriesterase, protein phosphatase, pyruvate kinase, recombinase A, sec-independent protein translocase, sll1032-protein, ThiF family protein, threonine dehydratase, threonine efflux protein, transcription factor, TTC1386-protein, ydr339c-protein, ydr507c-protein, and ymr013c-protein, respectively.

Moreover, the nucleic acid molecule of the invention can comprise only a portion of the coding region of one of the nucleic acid molecules depicted in Table I, application no. 9, columns 5 or 8, for example a fragment which can be used as a probe or primer or a fragment encoding a biologically active portion of the polypeptide of the present invention or of a polypeptide used in the process of the present invention, i.e. having above-mentioned activity, e.g. conferring the production or the increased production of the respective fine chemical linolenic acid as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted. The nucleotide sequences determined from the cloning of the present protein-according-to-the-inventionencoding gene allows for the generation of probes and primers designed for use in identifying and/or cloning its homologues from other cell types and organisms. The probe/primer typically comprises a substantially purified oligonucleotide. The oligonucleotide typically comprises a region of nucleotide sequence that hybridizes under stringent conditions to at least about 12, 15 preferably about 20 or 25, more preferably about 40, 50 or 75 consecutive nucleotides of a sense strand of one of the sequences set forth, e.g., in the respective line in Table I, application no. 9, column 5 or 8, preferably the coding region thereof, or a homolog or a fragment thereof, an anti-sense sequence of one of the sequences, e.g., set forth in the respective line in Table I, application no. 9, columns 5 or 8, preferably the coding region thereof, or a homolog or a fragment thereof or naturally occurring mutants thereof. Primers based on a nucleic acid molecule of the invention can be used in PCR reactions to clone homologs of the polypeptide of the invention or of the polypeptide used in the process of the invention, e.g. as the primers described in the examples of the present invention, e.g. as shown in the examples. A PCR with the primers shown in the respective line in Table III, column 8 will result in a fragment of the gene product as shown in Table II, application no. 9, column 5.

Primer sets are interchangeable. The person skilled in the art knows how to design and combine said primers to result in the desired product, e.g. in a full length clone or a partial sequence. Probes based on the sequences of the nucleic acid molecule of the invention or used in the process of the present invention can be used to detect transcripts or genomic sequences encoding the same or homologous proteins. The probe can further comprise a label group attached thereto, e.g. the label group can be a radioisotope, a fluorescent compound, an enzyme, or an enzyme co-factor. Such probes can be used as a part of a genomic marker test kit for identifying cells which express a polypeptide of the invention or used in the process of the present invention, such as by measuring a level of an encoding nucleic acid molecule in a sample of cells, e.g. detecting mRNA levels or determining, whether a genomic gene comprising the sequence of the polynucleotide of the invention or used in the processes of the present invention has been mutated or deleted.

As the expression of a respective nucleic acids of the invention is related to the synthesis of the respective fine chemical linolenic acid its function as a probe extends to the detection of microorganisms, plant tissues, plants, plant variets, plant ecotypes or plant genera with varying capability or potential for synthesis of the respective fine chemical linolenic acid. Therefore in one embodiment the present invention relates to a method for analyzing the capability or potential of a plant tissue, a plant, a plant variety or plant ecotype to produce the fine chemical linolenic acid by using the nucleic acid of the invention or parts thereof as a probe to detect the amount of the nucleic acid of the invention in the non-human organism or a part thereof in comparison to another non-human organism.

The nucleic acid molecule of the invention encodes a polypeptide or portion thereof which includes an amino acid sequence which is sufficiently homologous to the amino acid sequence shown in respective line in Table II, application no. 9, columns 5 or 8 such that the protein or portion thereof maintains the ability to confer the production or the increased production of the respective fine chemical linolenic acid as compared to a corresponding, e.g. non-transformed, wild type non-human organism, like a microorganism or a plant cell, plant or part thereof, in particular increasing the activity as mentioned above or as described in the examples in microorganisms or plants.

As used herein, the language “sufficiently homologous” refers to proteins or portions thereof which have amino acid sequences which include a minimum number of identical or equivalent amino acids (e.g. an amino acid which has a similar side chain as an amino acid in one of the sequences of the polypeptide of the present invention) to an amino acid sequence shown in the respective line in Table II, application no. 9, columns 5 or 8 such that the protein or portion thereof is able to confer the production or the increased production of the respective fine chemical linolenic acid as compared to a corresponding, e.g. non-transformed, wild type non-human organism, like a microorganism or a plant cell, plant or part thereof. For example having the activity of a protein as shown in the respective line in Table II, application no. 9, column 3 and as described herein.

In an embodiment of the present invention, the nucleic acid molecule of the present invention comprises a nucleic acid molecule that encodes a portion of the protein of the present invention. The protein is at least about 30%, 35%, 40%, 45% 50%, 55%, 60%, 65%, 70%, 75%, 80%, 85%, 90%, 91%, 92%, 93%, 94%, 95%, 96%, 97%, 98%, 99%, 99.5% or more homologous to an entire amino acid sequence shown in the respective line of Table II, application no. 9, column 5 or 8 and has the above-mentioned activity, e.g. conferring the production or the increased production of the respective fine chemical linolenic acid as compared to a corresponding, e.g. non-transformed, wild type non-human organism, like a microorganism or a plant cell, plant or part thereof by, for example in a embodiment expression either in the cytosol or in an organelle such as a plastid or mitochondria or both, preferably in a plastid, or in another embodiment by targeted or non-targeted expression.

[0338.1.8.9] to [0339.1.8.9] for the disclosure of these paragraphs see [0338.1.8.8] to [0339.1.8.8] above.

The invention further relates to nucleic acid molecules or methods using said nucleic acid molecules that differ from one of the nucleotide sequences shown in the respective line in Table I, application no. 9, columns 5 or 8, preferably the coding region thereof, or fragments or homologs thereof, due to degeneracy of the genetic code but encode a polypeptide of the present invention, in particular a polypeptide having the above-mentioned activity, e.g. as those polypeptides depicted in the respective line in Table II, application no. 9, columns 5 or 8, fragments or homologs thereof. Advantageously, the nucleic acid molecule of the invention comprises, or in an other embodiment has, a nucleotide sequence encoding a protein comprising, or in an other embodiment having, an amino acid sequence shown in the respective line in Table II, application no. 9, columns 5 or 8, fragments or homologs thereof. In a further embodiment, the nucleic acid molecule of the invention encodes a full length protein which is substantially homologous to an amino acid sequence shown in the respective line in Table II, application no. 9, columns 5 or 8, or homologs thereof. However, in an embodiment, the nucleic acid molecule of the present invention does not consist of the sequence shown in the respective line in Table I, preferably Table IA, application no. 9, columns 5 or 8, preferably the coding region thereof, but the functional homologs thereof.

[0341.1.8.9] to [0343.1.8.9] for the disclosure of these paragraphs see [0341.1.8.8] to [0343.1.8.8] above.

Accordingly, in another embodiment, a nucleic acid molecule of the invention is at least 15, 20, 25 or 30 nucleotides in length. Preferably, it hybridizes under stringent conditions to a nucleic acid molecule comprising a nucleotide sequence of the nucleic acid molecule of the present invention or used in the process of the present invention, e.g. comprising the sequence shown in the respective line in Table I, application no. 9, columns 5 or 8, preferably the coding region thereof. In another embodiment the nucleic acid molecule is preferably at least 20, 30, 50, 100, 250 or more nucleotides in length.

for the disclosure of this paragraph see [0345.1.8.8] above.

Preferably, a nucleic acid molecule of the invention that hybridizes under stringent conditions to a sequence shown in the respective line in Table I, application no. 9, columns 5 or 8, preferably the coding region thereof, corresponds to a naturally-occurring nucleic acid molecule of the invention. As used herein, a “naturally-occurring” nucleic acid molecule refers to a RNA or DNA molecule having a nucleotide sequence that occurs in nature (e.g. encodes a natural protein). Preferably, the nucleic acid molecule encodes a natural protein having above-mentioned activity, e.g. conferring the production or the increased production of the respective fine chemical linolenic acid as compared to a corresponding, e.g. non-transformed, wild type non-human organism, like a microorganism or a plant cell, plant or part thereof after increasing the expression or activity thereof or the activity of a protein of the invention or used in the process of the invention, in an embodiment for example expression either in the cytosol or in an organelle such as a plastid or mitochondria, preferably in plastids, or, in another embodiment by targeted or non-targeted expression.

for the disclosure of this paragraph see [0347.1.8.8] above.

For example, nucleotide substitutions leading to amino acid substitutions at “non-essential” amino acid residues can be made in a sequence of the nucleic acid molecule of the invention or used in the process of the invention, e.g. shown in the respective line in Table I, application no. 9, columns 5 or 8, preferably the coding region thereof, or fragments or homologs thereof.

[0349.1.8.9] to [0350.1.8.9] for the disclosure of these paragraphs see [0349.1.8.8] to [0350.1.8.8] above.

Accordingly, the invention relates to nucleic acid molecules encoding a polypeptide having above-mentioned activity, in a non-human organisms or parts thereof by for example expression either in the cytosol or in an organelle such as a plastid or mitochondria or both, preferably in plastids, that contain changes in amino acid residues that are not essential for said activity. Such polypeptides differ in amino acid sequence from a sequence depicted in the respective line in Table II, especially Table IIA, application no. 9, columns 5 or 8, but retain said activity described herein. The nucleic acid molecule can comprise a nucleotide sequence encoding a polypeptide, wherein the polypeptide comprises an amino acid sequence at least about 50% identical to an amino acid sequence shown in the respective line in Table II, application no. 9, columns 5 or 8, and is capable of conferring the production or the increased production of the respective fine chemical linolenic acid as compared to a corresponding, e.g. non-transformed, wild type non-human organism, like a microorganism or a plant cell, plant or part thereof, after increasing its activity for example in an embodiment by expression either in the cytosol or in an organelle such as a plastid or mitochondria or both, preferably in plastids, or, in another embodiment by targeted or non-targeted expression. Preferably, the protein encoded by the nucleic acid molecule is at least about 60%, 70%, 80%, 90%, 95%, 96%, 97%, 98%, 99% or 99.5% identical to the sequence shown in the respective line in Table II, application no. 9, columns 5 or 8.

[0352.1.8.9] to [0357.1.8.9] for the disclosure of these paragraphs see [0352.1.8.8] to [0357.1.8.8] above.

Functional equivalents of the nucleic acid molecules according to the present invention and/or used in the process according to the present invention represent an embodiment of homologs. Functional equivalents derived from the nucleic acid molecules as shown in the respective line in Table I, application no. 9, columns 5 or 8, preferably the coding region thereof, according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the nucleic acid molecules as shown in the respective line in Table I, application no. 9, columns 5 or 8, preferably the coding region thereof, according to the invention and encode polypeptides having essentially the same properties as the polypeptide as shown in the respective line in Table II, application no. 9, columns 5 or 8.

Functional equivalents derived from one of the polypeptides as shown in the respective line in Table II, application no. 9, columns 5 or 8 according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the polypeptides as shown in the respective line in Table II, application no. 9, columns 5 or 8 according to the invention and having essentially the same properties as the polypeptide as shown in the respective line in Table II, application no. 9, columns 5 or 8.

for the disclosure of this paragraph see [0359.1.8.8] above.

A nucleic acid molecule encoding a homolog to a polypeptide depicted in the respective line in Table II, application no. 9, columns 5 or 8 can be created by introducing one or more nucleotide substitutions, additions or deletions into the nucleic acid molecule of the present invention, in particular of Table I, application no. 9, columns 5 or 8, in the respective line, such that one or more amino acid substitutions, additions or deletions are introduced into the encoded protein. Mutations can be introduced into the nucleic acid molecules as depicted in the respective line in Table I, application no. 9, columns 5 or 8, preferably the coding region thereof, by standard techniques, such as site-directed mutagenesis, PCR-mediated mutagenesis or other methods known to the person skilled in the art.

[0361.1.8.9] to [0363.1.8.9] for the disclosure of these paragraphs see [0361.1.8.8] to [0363.1.8.8] above.

Homologues of the nucleic acid sequences used, with the sequences shown in the respective line in Table I, application no. 9, columns 5 or 8, preferably the coding region thereof, comprise also allelic variants with at least approximately 30%, 35%, 40%, 45%, 50%, 55%, 60%, 65%, 70%, 75%, 80%, 85%, 90%, 91%, 92%, 93%, 94% 95%. 96%, 97%, 98%, 99%, 99.5% or more % homology with one of the nucleotide sequences shown or the abovementioned derived nucleic acid sequences or their homologues or derivatives or parts of these. Allelic variants encompass in particular functional variants which can be obtained by deletion, insertion or substitution of nucleotides from the sequences shown in the respective line, preferably in Table I, columns 5 or 8, preferably the coding region thereof, or from the derived nucleic acid sequences, the intention being, however, that the enzymatic activity or the biological activity of the resulting proteins synthesized is advantageously retained or increased.

In one embodiment of the present invention, the nucleic acid molecule of the invention or used in the process of the invention comprises the sequences shown in the respective line in any of the Table I, application no. 9, columns 5 or 8, preferably the coding region thereof, fragments or homologs thereof. It is preferred that the nucleic acid molecule comprises as little as possible other nucleotides not shown in the respective line in any one of Table I, application no. 9, columns 5 or 8, preferably the coding region thereof, fragments or homologs thereof. In one embodiment, the nucleic acid molecule comprises less than 500, 400, 300, 200, 100, 90, 80, 70, 60, 50 or 40 further nucleotides. In a further embodiment, the nucleic acid molecule comprises less than 30, 20 or 10 further nucleotides. In one embodiment, the nucleic acid molecule used in the process of the invention is identical to the sequences shown in the respective line in Table I, application no. 9, columns 5 or 8, preferably the coding region thereof.

Also preferred is that the nucleic acid molecule used in the process of the invention encodes a polypeptide comprising the sequence shown in the respective line in Table II, application no. 9, columns 5 or 8, fragments or homologs thereof. In one embodiment, the nucleic acid molecule encodes less than 150, 130, 100, 80, 60, 50, 40 or 30 further amino acids. In a further embodiment, the encoded polypeptide comprises less than 20, 15, 10, 9, 8, 7, 6 or 5 further amino acids. In one embodiment used in the inventive process, the encoded polypeptide is identical to the sequences shown in the respective line in Table II, application no. 9, columns 5 or 8.

In one embodiment, the nucleic acid molecule of the invention or used in the process encodes a polypeptide comprising the sequence shown in the respective line in Table II, application no. 9, columns 5 or 8, or fragments or homologs thereof, and comprises less than 100 further nucleotides. In a further embodiment, said nucleic acid molecule comprises less than 30 further nucleotides. In one embodiment, the nucleic acid molecule used in the process is identical to a coding sequence of the sequences shown in the respective line in Table I, application no. 9, columns 5 or 8.

Polypeptides (=proteins), which still have the essential biological or enzymatic activity of the polypeptide of the present invention conferring the production or the increased production of the respective fine chemical linolenic acid as compared to a corresponding, e.g. non-transformed, wild type non-human organism, like a microorganism or a plant cell, plant or part thereof, i.e. whose activity is essentially not reduced, are polypeptides with at least 10%, 20%, 30%, 40%, 50%, 60%, 70%, 80%, 90%, 95% or more of the wild type biological activity or enzymatic activity, advantageously, the activity is essentially not reduced in comparison with the activity of a polypeptide shown in the respective line in Table II, application no. 9, columns 5 or 8 expressed under identical conditions.

Homologues of nucleic acid molecules shown in the respective line in Table I, application no. 9, columns 5 or 8, preferably the coding region thereof, or fragments thereof, or of the derived sequences shown in the respective line in Table II, application no. 9, columns 5 or 8, or fragments thereof, also mean truncated sequences, cDNA, single-stranded DNA or RNA of the coding and noncoding DNA sequence. Homologues of said sequences are also understood as meaning derivatives, which comprise noncoding regions such as, for example, UTRs, introns, terminators, enhancers or promoter variants. The promoters upstream of the nucleotide sequences stated can be modified by one or more nucleotide substitution(s), insertion(s) and/or deletion(s) without, however, interfering with the functionality or activity either of the promoters, the open reading frame (=ORF) or with the 3′-regulatory region such as terminators or other 3′-regulatory regions, which are far away from the ORF. It is furthermore possible that the activity of the promoters is increased by modification of their sequence, or that they are replaced completely by more active promoters, even promoters from heterologous organisms. Appropriate promoters are known to the person skilled in the art and are mentioned herein below.

[0370.1.8.9] to [0379.1.8.9] for the disclosure of these paragraphs see [0370.1.8.8] to [0379.1.8.8] above.

Moreover, a native polypeptide conferring the increase of the respective fine chemical linolenic acid in a non-human organism or a part thereof can be isolated from cells (e.g., endothelial cells), for example using the antibody of the present invention as described below, in particular, an antibody against proteins having 3-oxoacyl-[acyl-carrier-protein] reductase, 3-oxoacyl-[acyl-carrier-protein] synthase, AAS bifunctional protein, acid phosphatase precursor, acid shock protein, acyl transferase, acyl-CoA synthase, arginine decarboxylase, aromatic acid decarboxylase, asparaginase, At2g45420-protein, ATPase epsilon subunit, b0195-protein, b1234-protein, b1248-protein, b1280-protein, b1330-protein, b1470-protein, b1670-protein, b1672-protein, b1837-protein, b2032-protein, b2107-protein, b2399-protein, b2474-protein, b2613-protein, b2682-protein, b3442-protein, b3534-protein, b4023-protein, bifunctional purine biosynthesis protein, branched chain amino acid ABC transporter ATP-binding protein, branched-chain amino acid permease, carbamoyl-phosphate synthase subunit, carbon dioxide concentrating mechanism protein, cation-transporting ATPase, cell division control protein, coproporphyrinogen III oxidase, CTP synthase, CTP synthetase, dihydroxyacetone kinase, DnaJ-like chaperone, electron transport complex protein, ethanolamine utilization protein, F-box protein, flavodoxin, fumarate hydratase, glucose-6-phosphate 1-dehydrogenase, glutaredoxin, glycerol-3-phosphate dehydrogenase, heat shock transcription factor, HesB/YadR/YfhF family protein, isopropylmalate isomerase large subunit, metal-dependent hydrolase, molecular chaperone, monothiol glutaredoxin, multidrug resistance protein, oxidoreductase, permease protein of ABC transporter, phosphate import ATP-binding protein, phosphoadenosine phosphosulfate reductase , phosphofructokinase, phosphotriesterase, protein phosphatase, pyruvate kinase, recombinase A, sec-independent protein translocase, sll1032-protein, ThiF family protein, threonine dehydratase, threonine efflux protein, transcription factor, TTC1386-protein, ydr339c-protein, ydr507c-protein, or ymr013c-protein activity, respectively, or an antibody against polypeptides as shown in the respective line in Table II, application no. 9, columns 5 or 8, or fragments or homologs thereof which can be produced by standard techniques utilizing the polypeptid of the present invention or fragment thereof, i.e., the polypeptide of this invention (FCRP). Preferred are monoclonal antibodies.

for the disclosure of this paragraph see [0381.1.8.8] above.

In one embodiment, the present invention relates to a polypeptide as depicted in the respective line in Table II, especially in Table IIA or especially in Table IIB, application no. 9, columns 5 or 8, or fragment or homolog thereof or as coded by the nucleic acid molecule shown in the respective line in Table I, especially Table IA or especially Table IB, application no. 9, columns 5 or 8, preferably the coding region thereof, or fragment or homolog thereof.

In an embodiment, the present invention relates to a polypeptide comprising or consisting of a consensus sequence shown in the respective line in Table IV, application no. 9, column 8 or at least one polypeptide motif shown in the respective line in Table IV, application no. 9, column 8.

In an embodiment thereof, the present invention relates to a polypeptide comprising or consisting of a consensus sequence shown in the respective line in Table IV, application no. 9, column 8 or comprising at least one polypeptide motif shown in the respective line in Table IV, application no. 9, column 8, whereby not more than 20, 15, 10, 9, 8, 7, 6, 5, 4, 3, 2, or 1, or 0 of the amino acids can be replaced by any amino acid.

In another embodiment thereof, the present invention relates to a polypeptide comprising or consisting of a consensus sequence shown in the respective line in Table IV, application no. 9, column 8 or comprising at least one polypeptide motif shown in the respective line in Table IV, application no. 9, column 8, whereby not more than 15%, 10%, 5%, 4%, 3%, 2% or1% or 0% of the amino acids defined as distinct specific amino acids are/is replaced by another amino acid.

In another embodiment thereof, the present invention relates to a polypeptide comprising or consisting of a consensus sequence shown in the respective line in Table IV, application no. 9, column 8 or comprising at least one polypeptide motif shown in the respective line in Table IV, application no. 9, column 8, whereby not more than 20, 15, 10, 9, 8, 7, 6, 5, 4, 3, 2, or 1, or 0 amino acids are inserted into the consensus sequence or polypeptide motif.

In an embodiment of the present invention, the polypeptide of the present invention comprises or consists of a consensus sequence or at least one polypeptide motif shown in the respective line in Table IV, application no. 9, column 8, whereby said polypeptide distinguishes over the sequence depicted in the respective line in Table II, application no. 9, columns 5 or 8 by one or more amino acids. In one embodiment, said polypeptide of the invention distinguishes over the sequence shown in the respective line in Table II, application no. 9, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 9, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in the respective line in Table II, application no. 9, columns 5 and 8.

In an embodiment, the invention relates to a polypeptide conferring an increase in the respective fine chemical linolenic acid in a non-human organism, especially a microorganism or a plant, or a part thereof, being encoded by the nucleic acid molecule of the invention or used in the process of the invention and having a sequence which distinguishes over the sequence as shown in the respective line in Table II, application no. 9, columns 5 or 8 by one or more amino acids (but not exceeding 5%, preferably 4%, 3%, 2%, 1%, 0.5%, 0.2% or 0.1%). In an embodiment, said polypeptide of the invention does not comprise or consist of the sequence shown in the respective line in Table II, application no. 9, columns 5 or 8. In an embodiment, said polypeptide of the present invention is less than 100%, 99.999%, 99.99%, 99.9% or 99% identical. In one embodiment, said polypeptide which differs at least in one or more amino acids (but not exceeding 5%, preferably 4%, 3%, 2%, 1%, 0.5%, 0.2% or 0.1%) from the polypeptide shown in the respective line in Table II, application no. 9, columns 5 and 8 does not comprise a protein of the sequence shown in the respective line in Table II A and/or II B, application no. 9, columns 5 or 8.

In an embodiment, the present invention relates to a polypeptide having the activity of the protein as shown in the respective line in Table II, application no. 9, column 3, which distinguishes over the sequence depicted in the respective line in Table II, application no. 9, columns 5 or 8 by one or more amino acids, preferably by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 9, columns 5 and 8 by not more than 80%, 70%, 60%, 50%, 40%, 30%, 20%, 10%, 5%, 4%, 3%, 2%, 1% or 0.5%.

In another embodiment the polypeptide of the invention takes the form of a preprotein consisting of a plastidial or mitochondrial transit peptide joint to a polypeptide having the activity of the protein as shown in Table II, column 3, from which the transit peptide is preferably cleaved off upon transport of the preprotein into the organelle for example into the plastid or mitochondrion.

In another embodiment the polypeptide of the invention takes not the form of a preprotein.

[0390.1.8.9] to [0391.1.8.9] for the disclosure of these paragraphs see [0390.1.8.8] to [0391.1.8.8] above.

A polypeptide of the invention can participate in the process of the present invention. The polypeptide or a portion thereof comprises preferably an amino acid sequence, which is sufficiently homologous to an amino acid sequence shown in the respective line in Table II A and/or II B, application no. 9, columns 5 or 8.

Further, the polypeptide can have an amino acid sequence which is encoded by a nucleotide sequence which hybridizes, preferably hybridizes under stringent conditions as described above, to a nucleotide sequence of the nucleic acid molecule of the present invention. Accordingly, the polypeptide has an amino acid sequence which is encoded by a nucleotide sequence that is at least about 35%, 40%, 45%, 50%, 55%, 60%, 65%, 70%, 75%, 80%, 85%, 90%, 91%, 92%, 93%, 94%, 95%, 96%, 97%, 98%, 99%, 99.5% or more homologous to one of the amino acid sequences as shown in the respective line in Table II A and/or II B, application no. 9, columns 5 or 8, or fragments thereof. The preferred polypeptide of the present invention preferably possesses at least one of the activities according to the invention and described herein. A preferred polypeptide of the present invention includes an amino acid sequence encoded by a nucleotide sequence which hybridizes, preferably hybridizes under stringent conditions, to a nucleotide sequence shown in the respective line in Table I A and/or I B, application no. 9, columns 5 or 8, preferably the coding region thereof, or fragments thereto or which is homologous thereto, as defined above.

The polypeptide of the present invention can vary from the sequences shown in the respective line in Table II A and/or II B, application no. 9, columns 5 or 8 in amino acid sequence due to natural variation or mutagenesis, as described in detail herein. Accordingly, the polypeptide comprise an amino acid sequence which is at least about 35%, 40%, 45%, 50%, 55%, 60%, 65%, 70%, 75%, 80%, 85% or 90, 91%, 92%, 93%, 94%, 95%, 96%, 97%, 98%, 99%, 99.5% or more homologous to an entire amino acid sequence shown in the respective line in Table II A and/or II B, application no. 9, columns 5 or 8.

Biologically active portions of a polypeptide of the present invention include peptides comprising amino acid sequences derived from the amino acid sequence of the polypeptide of the present invention or used in the process of the present invention, e.g., the amino acid sequence shown in the respective line in Table II, application no. 9, columns 5 or 8 or the amino acid sequence of a protein homologous thereto, which include fewer amino acids than a full length polypeptide of the present invention or used in the process of the present invention or the full length protein which is homologous to a polypeptide of the present invention or used in the process of the present invention depicted herein, and exhibit at least one activity of a polypeptide of the present invention or used in the process of the present invention.

for the disclosure of this paragraph see [0396.1.8.8] above.

Manipulation of the nucleic acid molecule of the invention may result in the production of a protein having differences from the protein as shown in the respective line in Table II, application no. 9, column 5 or 8. Differences shall mean at least one amino acid different from the sequences as shown in the respective line in Table II, application no. 9, column 5 or 8, preferably at least 2, 3, 4, 5, 6, 7, 8, 9 or 10 amino acids (especially but not exceeding 0.1%, preferably 0.2%, 0.5%, 1%, 2%, 3% 4% or 5%), more preferably at least 15, 20, 25, 30, 35, 40, 45 or 50 amino acids (especially but not exceeding 0.5%, preferably 1%, 2%, 3% 4%, 5%, 10% or 25%) different from the sequences as shown in the respective line in Table II, application no. 9, column 5 or 8. These proteins may be improved in efficiency or activity, or may be more stable and therefore present in greater numbers in the cell as compared to the wild-type cell.

Any mutagenesis strategies for the polypeptide of the present invention or the polypeptide used in the process of the present invention to result in increasing said activity are not meant to be limiting; variations on these strategies will be readily apparent to one skilled in the art. Using such strategies, and incorporating the mechanisms disclosed herein, the nucleic acid molecule and polypeptide of the invention may be utilized to generate algae, ciliates, fungi, other microorganisms like C. glutamicum or plants or parts thereof, expressing proteins as shown in the respective line in Table II, application no. 9, column 5 or 8, or mutated proteins thereof. The nucleic acid molecules and polypeptide molecules of the invention are expressed such that the yield, production, and/or efficiency of production of a desired compound is improved.

[0399.1.8.9] to [0401.1.8.9] for the disclosure of these paragraphs see [0399.1.8.8] to [0401.1.8.8] above.

In one embodiment, a protein (=polypeptide) as shown in the respective line in Table II, application no. 9, column 5 or 8 refers to a polypeptide having an amino acid sequence corresponding to the polypeptide of the invention, especially to a polypeptide as shown in Table II, application no. 9, columns 5 or 8 or homologs thereof, or used in the process of the invention, whereas a “non-inventive protein or polypeptide” or “other polypeptide” refers to a polypeptide having an amino acid sequence corresponding to a protein which is not substantially homologous to a polypeptide of the invention, preferably which is not substantially homologous to a polypeptide or protein as shown in the respective line in Table II, application no. 9, column 5 or 8 or homologs thereof, e.g., a protein which does not confer the activity described herein and which is derived from the same or a different organism.

[0403.1.8.9] to [0409.1.8.9] for the disclosure of these paragraphs see [0403.1.8.8] to [0409.1.8.8] above.

As the nucleic acid molecules of the invention and/or the expression thereof is related to the synthesis of the respective fine chemical linolenic acid its function as a probe extends to the detection of microorganisms, plant tissues, plants, plant variets, plant ecotypes or plant genera with varying, advantageously increased, capability or potential for synthesis of the respective fine chemical linolenic acid. Therefore in one embodiment the present invention relates to a method for analyzing the capability or potential of a plant tissue, a plant, a plant variety or ecotype to produce the respective fine chemical linolenic acid by using the respective antibody of the invention as a probe to detect the amount of the polypeptide encoded by said nucleic acid molecule of the invention in a non-human organism in comparison to another organism.

Therefore in one embodiment the invention relates to a method to probe a non-human organism for its capability for the production of the respective fine chemical comprising the steps of
a) analyzing the sequence and/or the expression of the respective nucleic acid molecule of the invention in least two non-human organisms which differ in the capability to produce the respective fine chemical;
b) associate the different sequence and/or expression level of the nucleic acid molecules of the invention with their different capability to produce the respective fine chemical;
c) analyze the sequence and/or the expression of the nucleic acid molecule of the invention in at least a third non-human organism;
d) predict the capability of the at least third organism to produce the respective fine chemical based by comparing the sequence and/or expression of the nucleic acid of the invention to the sequences and/or expression levels of the nucleic acid molecule of the invention of the at least two organisms analyzed in step a.

[0411.1.8.9] to [0430.1.8.9] for the disclosure of these paragraphs see [0411.1.8.8] to [0430.1.8.8] above.

Accordingly the present invention relates to any cell transgenic for any nucleic acid characterized as part of the invention, e.g. conferring the increase of the respective fine chemical linolenic acid in a cell or a non-human organism or a part thereof, e.g. the nucleic acid molecule of the invention, the nucleic acid construct of the invention, the vector of the invention, the expression cassette according to the invention, or a nucleic acid molecule encoding the polypeptide of the invention, e.g. encoding a polypeptide having an activity as the protein as shown in the respective line in Table II, application no. 9, column 3. Due to the abovementioned activity the respective fine chemical linolenic acid content in a cell or a non-human organism is increased. For example, due to modulation or manipulation, the cellular activity is increased, in a preferred embodiment in organelles such as plastids or mitochondria, e.g. due to an increased expression or specific activity or specific targeting of the subject matters of the invention in a cell or a non-human organism or a part thereof especially in organelles such as plastids or mitochondria, or in another embodiment in the cytosol. Transgenic for a polypeptide having a protein or a protein activity means herein that due to modulation or manipulation of the genome, the activity of protein as shown in the respective line in Table II, application no. 9, column 3 or a protein as shown in the respective line in Table II, application no. 9, column 3-like activity is increased in the cell or non-human organism or part thereof, especially in organelles such as plastids or mitochondria, or especially in the cytosol. Examples are described above in context with the process of the invention.

for the disclosure of this paragraph see [0432.1.8.8] above.

A naturally occurring expression cassette—for example the naturally occurring combination of the promoter of the gene encoding a protein as shown in the respective line in Table II, application no. 9, column 5 or 8, preferably the coding region thereof, or fragments or homologs thereof with the corresponding encoding gene—becomes a transgenic expression cassette when it is modified by non-natural, synthetic “artificial” methods such as, for example, mutagenesis. Such methods have been described (U.S. Pat. No. 5,565,350; WO 00/15815; also see above).

[0434.1.8.9] to [0435.1.8.9] for the disclosure of these paragraphs see [0434.1.8.8] to [0435.1.8.8] above.

Transgenic plants comprising the respective fine chemical(s) synthesized in the process according to the invention can be marketed directly without isolation of the fine chemical synthesized. In the process according to the invention, plants are understood as meaning all plant parts, plant organs such as leaf, stalk, root, tubers or seeds or propagation material or harvested material or the intact plant. In this context, the seed encompasses all parts of the seed such as the seed coats, epidermal cells, seed cells, endosperm or embryonic tissue. The fine chemical produced in the process according to the invention may, however, also be isolated from the plant. In case of linolenic acid this can be in free form or bound to proteins. Fine chemical(s) produced by this process can be harvested by harvesting the non-human organisms either from the culture in which they grow or from the field. For example, this can be done via squeezing, grinding and/or extraction, salt precipitation and/or ion-exchange chromatography of the plant parts, preferably the plant seeds, plant fruits, plant tubers and the like.

[0437.1.8.9] to [0454.1.8.9] for the disclosure of these paragraphs see [0437.1.8.8] to [0454.1.8.8] above.

In one embodiment, the present invention relates to a method for the identification of a gene product conferring the production of or an increase in the fine chemical production in a non-human cell, comprising the following steps:
(a) contacting, e.g. hybridizing, a, some or all nucleic acid molecules of a sample, e.g. cells, tissues, plants or microorganisms or a nucleic acid library, which can contain a candidate gene encoding a gene product conferring the production of or an increase in the fine chemical after expression, with the nucleic acid molecule of the present invention as shown in the respective line in column 5 or 8 of Table I A or B of application no. 9, preferably the coding region thereof, or a fragment or homolog thereof;
(b) identifying the nucleic acid molecules, which hybridize under relaxed stringent conditions with the nucleic acid molecule of the present invention in particular to the nucleic acid molecule sequence shown in the respective line in Table I, application no. 9, columns 5 or 8, preferably the coding region thereof, of a fragment or a homolog thereof, preferably in Table I B, application no. 9, columns 5 or 8, preferably the coding region thereof, of a fragment or a homolog thereof, and, optionally, isolating the full length cDNA clone or complete genomic clone;
(c) introducing the candidate nucleic acid molecules or a fragment thereof in host cells, preferably in a plant cell or a microorganism, appropriate for producing the fine chemical;
(d) expressing the identified nucleic acid molecules in the host cells for which the production of or the increased production of the fine chemical is desired;
(e) assaying the fine chemical level in the non-human host cells; and
(f) identifying the nucleic acid molecule and/or its gene product which expression confers the production of or an increase in the fine chemical level in the non-human host cell after expression compared to the wild type.

for the disclosure of this paragraph see [0456.1.8.8] above.

In another embodiment, the present invention relates to a method for the identification of a gene product conferring the production of or an increase in the fine chemical production in a cell, comprising the following steps:
(a) identifying a nucleic acid molecule of a non-human organism, which is at least 20%, 25%, 30%, 35%, 40%, 50%, 60%, 70%, 80%, 90%, 95%, 96%, 97%, 98%, 99%, 99.5% or more homolog to the nucleic acid molecule encoding a polypeptide comprising the polypeptide molecule as shown in the respective line in column 5 or 8 of Table II, application no. 9, or comprising a consensus sequence or a polypeptide motif as shown in the respective line in column 8 of Table IV, application no. 9, or being encoded by a nucleic acid molecule comprising a polynucleotide as shown in the respective line in column 5 or 8 of Table I, application no. 9, preferably the coding region thereof, or a homologue thereof as described herein, for example via homology search in a data bank;
(b) introducing the candidate nucleic acid molecule in non-human host cells, preferably in a plant cell or a microorganism, appropriate for producing the fine chemical;
(c) expressing the identified nucleic acid molecule in the non-human host cells;
(d) assaying the fine chemical level in the non-human host cells; and
(e) identifying the nucleic acid molecule and its gene product which expression confers the production of or an increase in the fine chemical level in the non-human host cell after expression compared to the wild type.

for the disclosure of this paragraph see [0458.1.8.8] above.

Further, the nucleic acid molecules disclosed herein, in particular the nucleic acid molecules shown in the respective line in column 5 or 8 of Table I A or B of application no. 9, may be sufficiently homologous to the sequences of related species such that these nucleic acid molecules may serve as markers for the construction of a genomic map in related organism or for association mapping. Furthermore natural variation in the genomic regions corresponding to nucleic acids disclosed herein, in particular the nucleic acid molecule shown in the respective line in column 5 or 8 of Table I A or B, of application no. 9 or homologous thereof, may lead to variation in the activity of the proteins disclosed herein, in particular the proteins comprising polypeptides as shown in the respective lines in column 5 or 8 of Table II A or B, of application no. 9 or their homologous, or comprising the consensus sequence or the polypeptide motif as shown in the respective line in column 8 of Table IV, and in consequence in a natural variation of an increased fine chemical production.

In consequence natural variation eventually also exists in form of more active allelic variants leading already to a relative increase in the fine chemical. Different variants of the nucleic acid molecules disclosed herein, in particular the nucleic acid molecules comprising the nucleic acid molecules as shown in the respective line in column 5 or 8 of Table I A or B of application no. 9, preferably the coding region thereof, which corresponds to different levels of increase in fine chemical, e.g. different levels of increase in fine chemical, can be indentified and used for marker assisted breeding for an enhanced production of the fine chemical.

Natural variation may also exist in the regulatory regions, e.g. the promoters of the natural genes comprising the nucleic acid molecules as shown in the respective line column 5 or 8 of Table I A or B of application no. 9, preferably the coding region thereof, which can similarly correspond to different levels of increase in the fine chemical, e.g. different levels of increase in the fine chemical can be identified and used for marker assisted breeding for an enhanced production of the fine chemical.

Accordingly, the present invention relates to a method for breeding plants for the production of the fine chemical, comprising
(a) selecting a first plant variety with an increased production of the fine chemical, e.g. based on an increased expression of a nucleic acid molecule of the invention as disclosed herein, in particular of a nucleic acid molecule comprising a nucleic acid molecule as shown in the respective line in column 5 or 8 of Table I A or B of application no. 9, preferably the coding region thereof, or fragments or homologs thereof, or a polypeptide comprising a polypeptide as shown in the respective line in column 5 or 8 of Table II A or B of application no. 9, or fragments or homologs thereof, or comprising a consensus sequence or a polypeptide motif as shown in the respective line in column 8 of Table IV, or a homolog thereof, as described herein;
(b) associating the level of increased production of the fine chemical, with the expression level or the genomic structure of a gene encoding said polypeptide or said nucleic acid molecule;
(c) crossing the first plant variety with a second plant variety, which significantly differs in its level of fine chemical; and
(d) identifying, which of the offspring varieties has got increased production of the fine chemical by the expression level of said polypeptide or nucleic acid molecule or the genomic structure of the gene(s) encoding said polypeptide or nucleic acid molecule of the invention.

[0462.1.8.9] to [0482.1.8.9] for the disclosure of these paragraphs see [0462.1.8.8] to [0482.1.8.8] above.

In a further embodiment the present invention is characterized by the features as disclosed in the following items:

Item 1. A process for the production of the fine chemical linolenic acid
(a) increasing or generating one or more activities selected from the group consisting of 3-oxoacyl-[acyl-carrier-protein] reductase, 3-oxoacyl-[acyl-carrier-protein] synthase, AAS bifunctional protein, acid phosphatase precursor, acid shock protein, acyl transferase, acyl-CoA synthase, arginine decarboxylase, aromatic acid decarboxylase, asparaginase, At2g45420-protein, ATPase epsilon subunit, b0195-protein, b1234-protein, b1248-protein, b1280-protein, b1330-protein, b1470-protein, b1670-protein, b1672-protein, b1837-protein, b2032-protein, b2107-protein, b2399-protein, b2474-protein, b2613-protein, b2682-protein, b3442-protein, b3534-protein, b4023-protein, bifunctional purine biosynthesis protein, branched chain amino acid ABC transporter ATP-binding protein, branched-chain amino acid permease, carbamoyl-phosphate synthase subunit, carbon dioxide concentrating mechanism protein, cationtransporting ATPase, cell division control protein, coproporphyrinogen III oxidase, CTP synthase, CTP synthetase, dihydroxyacetone kinase, DnaJ-like chaperone, electron transport complex protein, ethanolamine utilization protein, F-box protein, flavodoxin, fumarate hydratase, glucose-6-phosphate 1-dehydrogenase, glutaredoxin, glycerol-3-phosphate dehydrogenase, heat shock transcription factor, HesB/YadR/YfhF family protein, isopropylmalate isomerase large subunit, metal-dependent hydrolase, molecular chaperone, monothiol glutaredoxin, multidrug resistance protein, oxidoreductase, permease protein of ABC transporter, phosphate import ATP-binding protein, phosphoadenosine phosphosulfate reductase , phosphofructokinase, phosphotriesterase, protein phosphatase, pyruvate kinase, recombinase A, sec-independent protein translocase, sll1032-protein, ThiF family protein, threonine dehydratase, threonine efflux protein, transcription factor, TTC1386-protein, ydr339c-protein, ydr507c-protein, and ymr013c-protein, in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of linolenic acid or a composition comprising linolenic acid in said non-human organism or in the culture medium surrounding said non-human organism.

Item 2. A process for the production of the respective fine chemical linolenic acid, which comprises
(A)
(i) increasing or generating of the expression of; and/or
(ii) increasing or generating the expression of an expression product of; and/or
(iii) increasing or generating one or more activities of an expression product encoded by;
at least one respective nucleic acid molecule comprising a nucleic acid molecule selected from the group consisting of:
(a) a nucleic acid molecule encoding the polypeptide shown in column 5 or 8 of Table II, preferably Table II B, application no.9, or a homolog or a fragment thereof;
(b) a nucleic acid molecule shown in column 5 or 8 of Table I, preferably Table I B, application no.9, or a homolog or a fragment thereof (preferably the coding region thereof);
(c) a nucleic acid molecule, which, as a result of the degeneracy of the genetic code, can be derived from a polypeptide sequence depicted in column 5 or 8 of Table II, preferably Table II B, application no.9;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 9, or the coding region thereof;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no.9;
(i) a nucleic acid molecule encoding a polypeptide having the activity represented by a respective protein as depicted in column 5 of Table II, application no. 9;
(j) a nucleic acid molecule which comprises a polynucleotide, which is obtained by amplifying a cDNA library or a genomic library using the primers as depicted in column 8 of Table III, application no. 9; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto;
in a non-human organism or a part thereof, preferably a microorganism, a plant cell, a plant or a part thereof, as compared to a corresponding non-transformed wild type non-human organism or a part thereof; and
(B) growing the non-human organism or a part thereof under conditions which permit the production of linolenic acid or a composition comprising linolenic acid in said non-human organism or in the culture medium surrounding said non-human organism.

Item 3. A process of items 1 or 2, comprising of recovering linolenic acid in its free or bound form.

Item 4. The process of any one of items 1 to 3, comprising the following steps:
(a) selecting a non-human organism or a part thereof expressing a polypeptide encoded by the nucleic acid molecule characterized in item 2;
(b) mutagenizing the selected non-human organism or the part thereof;
(c) comparing the activity or the expression level of said polypeptide in the mutagenized non-human organism or the part thereof with the activity or the expression of said polypeptide of the selected non-human organisms or the part thereof;
(d) selecting the mutated non-human organisms or parts thereof, which comprise an increased activity or expression level of said polypeptide compared to the selected non-human organism or the part thereof;
(e) optionally, growing and cultivating the non-human organisms or the parts thereof;
and
(f) recovering, and optionally isolating, the free or bound linolenic acid produced by the selected mutated non-human organisms or parts thereof.

Item 5. The process of any one of items 1 to 4 wherein the activity of said protein or the expression of said nucleic acid molecule is increased or generated transiently or stably.

Item 6. An isolated nucleic acid molecule comprising a nucleic acid molecule selected from the group consisting of:
(a) a nucleic acid molecule encoding a polypeptide shown in Table II, application no. 9, column 5 or 8, preferably shown in Table II B, application no. 9, column 8, or a homolog or a fragment thereof;
(b) a nucleic acid molecule shown in Table I, application no. 9, column 5 or 8, preferably shown in Table I B, application no. 9, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 9, preferably in column 8 of Table II B, application no. application no.;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 3, preferably shown in column 8 of Table I B, application no. 9, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 9;
(i) a nucleic acid molecule encoding a polypeptide having the activity represented by a respective protein as depicted in column 5 of Table II, application no. 9;
(j) a nucleic acid molecule comprising a polynucleotide which is obtained by amplifying a cDNA library or a genomic library using the primers in Table III, application no. 9, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (i),
or a nucleic acid molecule comprising a sequence which is complementary thereto.

Item 7. A nucleic acid construct which confers the expression of the nucleic acid molecule as defined in item 6, comprising one or more regulatory elements.

Item 8. An expression cassette comprising
(a) a promoter, preferably selected from the group consisting of Big35S, PCUbi, Super and USP;
(b) a nucleic acid molecule as defined in item 6.

Item 9. A vector comprising the nucleic acid molecule as defined in item 6 or the nucleic acid construct as claimed in item 7 or the expression cassette as claimed in item 8.

Item 10. A host cell or a plant organelle, which has been transformed with the vector as claimed in item 9 or the nucleic acid molecule as defined in item 6 or the nucleic acid construct as claimed in item 7 or the expression cassette as claimed in item 8.

Item 11. The host cell of item 10, which is a plant cell or a microorganism.

Item 12. A process for producing a polypeptide, wherein the polypeptide is expressed in a host cell as claimed in item 10 or 11.

Item 13. A polypeptide produced by the process as claimed in item 12 or encoded by the nucleic acid molecule as defined in item 6.

Item 14. An antibody, which binds specifically to the polypeptide as claimed in item 13.

Item 15. A plant tissue, propagation material, harvested material or a plant comprising the host cell as claimed in item 10 or 11.

Item 16. A process for the identification of a compound conferring an increase in linolenic acid production in a non-human organism, comprising the steps:
(a) culturing a plant cell or tissue or microorganism or maintaining a plant expressing the polypeptide encoded by the nucleic acid molecule as defined in item 6 conferring an increase in the amount of linolenic acid in a non-human organism or a part thereof and a readout system capable of interacting with the polypeptide under suitable conditions which permit the interaction of the polypeptide with said readout system in the presence of a compound or a sample comprising a plurality of compounds and capable of providing a detectable signal in response to the binding of a compound to said polypeptide under conditions which permit the expression of said readout system and of the polypeptide encoded by the nucleic acid molecule of item 6 conferring an increase in the amount of linolenic acidin a non-human organism or a part thereof;
(b) identifying if the compound is an effective agonist by detecting the presence or absence or increase of a signal produced by said readout system.

Item 17. A method for the production of an agricultural composition comprising the steps of the method of item 16 and formulating the compound identified in item 16 in a form acceptable for an application in agriculture.

Item 18. A composition comprising the nucleic acid molecule as defined in item 6, the polypeptide as claimed in item 13, the nucleic acid construct as claimed in item 7, the expression cassette as claimed in item 8, the vector as claimed in item 9, the compound as claimed in item 16, the antibody as claimed in item 14, and optionally an agricultural acceptable carrier.

Item 19. Use of the nucleic acid molecule as defined in item 6 for the identification of a nucleic acid molecule conferring an increase in linolenic acid after expression.

Item 20. Cosmetic, pharmaceutical, food or feed composition comprising the nucleic acid molecule as defined in item 6, the polypeptide as claimed in item 13, the nucleic acid construct as claimed in item 7, the expression cassette as claimed in item 8, the vector as claimed in item 9, the antibody as claimed in item 14 , the plant or a part thereof, or plant tissue as claimed in item 15, the harvested material or as claimed in item 15 or the host cell of item as claimed in items 10 or 11.

Item 21. Use of the nuclei acid molecule as defined in item 6, the polypeptide as claimed in item 13, the nucleic acid construct as claimed in item 7, the expression cassette as claimed in item 8, the vector as claimed in claim 9, the plant or plant tissue as claimed in item 15, or the host cell of item 10 to 11 for the production of plant resistant to a herbicide inhibiting the production of linolenic acid.

[0483.1.8.9] to [0494.1.9.9] for the disclosure of these paragraphs see [0483.1.8.8] to [0494.1.9.8] above.

### Example 11a

### PCR Amplification of the Sequences

Unless otherwise specified, standard methods as described in Sambrook et al., Molecular Cloning: A laboratory manual, Cold Spring Harbor 1989, Cold Spring Harbor Laboratory Press are used.

The inventive sequences as shown in the respective line in Table I, column 5, preferably the coding region thereof, were amplified by PCR as described in the protocol of the Pfu Ultra, Pfu Turbo or Herculase DNA polymerase (Stratagene). The composition for the protocol of the Pfu Ultra, Pfu Turbo or Herculase DNA polymerase was as follows: 1× PCR buffer (Stratagene), 0.2 mM of each dNTP, 100 ng genomic DNA of Saccharomyces cerevisiae (strain S288C; Research Genetics, Inc., now Invitrogen), Escherichia coli (strain MG1655; E. coli Genetic Stock Center), Synechocystis sp. (strain PCC6803), Azotobacter vinelandii (strain N.R. Smith,16), Thermus thermophilus (HB8) or 50 ng cDNA from various tissues and development stages of Arabidopsis thaliana (ecotype Columbia), Physcomitrella patens, Glycine max (variety Resnick), Brassica napus, Oryza sativa or Zea mays (variety B73, Mo17, A188), 50 pmol forward primer, 50 pmol reverse primer, with or without 1 M Betaine, 2.5 u Pfu Ultra, Pfu Turbo or Herculase DNA polymerase. The amplification cycles were as follows: 1 cycle of 2-3 minutes at 94-95° C., then 25-36 cycles with 30-60 seconds at 94-95° C., 30-45 seconds at 50-60° C. and 210-480 seconds at 72° C., followed by 1 cycle of 5-10 minutes at 72° C., then 4-16° C.—preferably for Saccharomyces cerevisiae, Escherichia coli, Synechocystis sp., Azotobacter vinelandii, Thermus thermophilus.

In case of Arabidopsis thaliana, Brassica napus, Glycine max, Oryza sativa, Physcomitrella patens, Zea mays the amplification cycles were as follows: 1 cycle with 30 seconds at 94° C., 30 seconds at 61° C., 15 minutes at 72° C., then 2 cycles with 30 seconds at 94° C., 30 seconds at 60° C., 15 minutes at 72° C., then 3 cycles with 30 seconds at 94° C., 30 seconds at 59° C., 15 minutes at 72° C., then 4 cycles with 30 seconds at 94° C., 30 seconds at 58° C., 15 minutes at 72° C., then 25 cycles with 30 seconds at 94° C., 30 seconds at 57° C., 15 minutes at 72° C., then 1 cycle with 10 minutes at 72° C., then finally 4-16° C.

RNAs were generated with the RNeasy Plant Kit according to the standard protocol (Qiagen) and Superscript II Reverse Transkriptase was used to produce double stranded cDNA according to the standard protocol (Invitrogen).

ORF specific primer pairs for the genes to be expressed are shown in the respective line in Table III, column 8. The following adapter sequences were added to Saccharomyces cerevisiae ORF specific primers (see Table III) for cloning purposes:

SEQ ID NO: 20

| | | |
|---|---|---|
| i) | foward primer: | 5′-GGAATTCCAGCTGACCACC-3′ |

SEQ ID NO: 21

| | | |
|---|---|---|
| ii) | reverse primer: | 5′-GATCCCCGGGAATTGCCATG-3′ |

These adaptor sequences allow cloning of the ORF into the various vectors containing the Resgen adaptors, see table column E of Table c.

The following adapter sequences were added to Saccharomyces cerevisiae, Escherichia coli, Synechocystis sp., Azotobacter vinelandii, Thermus thermophilus, Arabidopsis thaliana, Brassica napus, Glycine max, Oryza sativa , Physcomitrella patens, or Zea mays ORF specific primers for cloning purposes:

| SEQ ID NO: 22 | | |
|---|---|---|
| iii) | forward primer: | 5′-TTGCTCTTCC- 3′ |
| SEQ ID NO: 23 | | |
| iiii) | reverse primer: | 5′-TTGCTCTTCG-3′ |

The adaptor sequences allow cloning of the ORF into the various vectors containing the Colic adaptors, see table column E of Table c.

Therefore for amplification and cloning of Saccharomyces cerevisiae SEQ ID NO: 90165, a primer consisting of the adaptor sequence i) and the ORF specific sequence SEQ ID NO: 90175 and a second primer consisting of the adaptor sequence ii) and the ORF specific sequence SEQ ID NO: 90176 were used.

For amplification and cloning of Escherichia coli SEQ ID NO: 8920, a primer consisting of the adaptor sequence iii) and the ORF specific sequence SEQ ID NO: 8930 and a second primer consisting of the adaptor sequence iiii) and the ORF specific sequence SEQ ID NO: 8931 were used.

For amplification and cloning of Synechocystis sp. SEQ ID NO: 11471, a primer consisting of the adaptor sequence iii) and the ORF specific sequence SEQ ID NO: 11983 and a second primer consisting of the adaptor sequence iiii) and the ORF specific sequence SEQ ID NO: 11984 were used.

For amplification and cloning of Arabidopsis thaliana SEQ ID NO: 1061, a primer consisting of the adaptor sequence iii) and the ORF specific sequence SEQ ID NO: 1293 and a second primer consisting of the adaptor sequence iiii) and the ORF specific sequence SEQ ID NO: 1294 were used.

For amplification and cloning of Azotobacter vinelandii SEQ ID NO: 26196, a primer consisting of the adaptor sequence iii) and the ORF specific sequence SEQ ID NO: 26428 and a second primer consisting of the adaptor sequence iiii) and the ORF specific sequence SEQ ID NO: 26429 were used.

For amplification and cloning of Glycine max SEQ ID NO: 10811, a primer consisting of the adaptor sequence iii) and the ORF specific sequence SEQ ID NO: 11207 and a second primer consisting of the adaptor sequence iiii) and the ORF specific sequence SEQ ID NO: 11208 were used.

For amplification and cloning of Thermus thermophilus SEQ ID NO: 61553, a primer consisting of the adaptor sequence iii) and the ORF specific sequence SEQ ID NO: 61719 and a second primer consisting of the adaptor sequence iiii) and the ORF specific sequence SEQ ID NO: 61720 were used.

Following these examples every sequence disclosed in Table I, preferably column 5, especially the coding region thereof can be cloned by fusing the adaptor sequences to the respective specific primers sequences as disclosed in Table III, column 8 using the respective vectors shown in Table c.

[0496.1.8.9] to [0499.1.8.9] for the disclosure of these paragraphs see [0496.1.8.8] to [0499.1.8.8] above.

### Example 11e

### Cloning of Inventive Sequences as Shown in Table I, Column 5 in the Different Expression Vectors

For cloning of for example SEQ ID NO: 8920 from Escherichia coli or any other ORF from Escherichia coli, Synechocystis sp., Azotobacter vinelandii, Thermus thermophilus, Arabidopsis thaliana, or Glycine max the vector DNA was treated with the restriction enzymes Pacl and Ncol following the standard protocol (MBI Fermentas).

For cloning for example the ORFs of SEQ ID NO: 90165 from Saccharomyces cerevisiae or any other ORF from Saccharomyces cerevisiae into vectors containing the Resgen adaptor sequence the respective vector DNA was treated with the restriction enzyme Ncol.

For cloning of for example ORFs SEQ ID NO: 119408 from Saccharomyces cerevisiae or any other ORF from Saccharomyces cerevisiae into vectors containing the Colic adaptor sequence, the respective vector DNA was treated with the restriction enzymes Pacl and Ncol following the standard protocol (MBI Fermentas).

In all cases the reaction was stopped by inactivation at 70° C. for 20 minutes and purified over QlAquick or NucleoSpin Extract II columns following the standard protocol (Qiagen or Macherey-Nagel).

Then the PCR-product representing the amplified ORF with the respective adapter sequences and the vector DNA were treated with T4 DNA polymerase according to the standard protocol (MBI Fermentas) to produce single stranded overhangs with the parameters 1 unit T4 DNA polymerase at 37° C. for 2-10 minutes for the vector and 1-2 u T4 DNA polymerase at 15-17° C. for 10-60 minutes for the PCR product representing SEQ ID NO: 8920.

The reaction was stopped by addition of high-salt buffer and purified over QlAquick or NucleoSpin Extract II columns following the standard protocol (Qiagen or Macherey-Nagel).

According to this example the skilled person is able to clone all sequences disclosed in Table I, preferably column 5 or column 8, especially the coding region thereof.

Approximately 30-60 ng of prepared vector and a defined amount of prepared amplificate were mixed and hybridized at 65° C. for 15 minutes followed by 37° C. 0.1 ° C/1 seconds, followed by 37° C. 10 minutes, followed by 0.1 ° C/1 seconds, then 4-10 ° C.

The ligated constructs were transformed in the same reaction vessel by addition of competent E. coli cells (strain DHSalpha) and incubation for 20 minutes at 1° C. followed by a heat shock for 90 seconds at 42° C. and cooling to 1-4° C. Then, complete medium (SOC) was added and the mixture was incubated for 45 minutes at 37° C. The entire mixture was subsequently plated onto an agar plate with 0.05 mg/ml kanamycin and incubated overnight at 37° C.

The outcome of the cloning step was verified by amplification with the aid of primers which bind upstream and downstream of the integration site, thus allowing the amplification of the insertion. The amplifications were carried out as described in the protocol of Taq DNA polymerase (Gibco-BRL).

The amplification cycles were as follows:

1 cycle of 1-5 minutes at 94° C., followed by 35 cycles of in each case 15-60 seconds at 94° C., 15-60 seconds at 50-66° C. and 5-15 minutes at 72° C., followed by 1 cycle of 10 minutes at 72° C., then 4-16° C.

Several colonies were checked, but only one colony for which a PCR product of the expected size was detected was used in the following steps.

A portion of this positive colony was transferred into a reaction vessel filled with complete medium (LB) supplemented with kanamycin and incubated overnight at 37° C.

The plasmid preparation was carried out as specified in the Qiaprep or NucleoSpin Multi-96 Plus standard protocol (Qiagen or Macherey-Nagel).

[501.1.8.9] to [0503.1.8.9] for the disclosure of these paragraphs see [0501.1.8.8] to [0503.1.8.8] above. Table d showing results of plant analyses

TABLE d

| SeqID | Target | Locus | Metabolite | Source | Promotor | Method | Min | Max |
|---|---|---|---|---|---|---|---|---|
| 17451 | non- | At1g07430 | linolenic | ARA_LEAF | p-PcUBI | GC | 12 | 52 |
| | targeted | | acid | | | | | |
| 1061 | non- | At1g68320 | linolenic | ARA_LEAF | p-PcUBI | GC | 15 | 74 |
| | targeted | | acid | | | | | |
| 19419 | non- | At1g72770 | linolenic | ARA_LEAF | p-PcUBI | GC | 21 | 49 |
| | targeted | | acid | | | | | |
| 113714 | plastidic | At2g36580 | linolenic | ARA_LEAF | p-PcUBI | GC | 12 | 22 |
| | | | acid | | | | | |
| 1623 | non- | At2g45420 | linolenic | ARA_LEAF | p-PcUBI | GC | 13 | 27 |
| | targeted | | acid | | | | | |
| 68657 | non- | At3g02990 | linolenic | ARA_LEAF | p-PcUBI | GC | 14 | 39 |
| | targeted | | acid | | | | | |
| 2935 | non- | At3g62950 | linolenic | ARA_LEAF | p-PcUBI | GC | 14 | 36 |
| | targeted | | acid | | | | | |
| 3279 | non- | At4g15670 | linolenic | ARA_LEAF | p-PcUBI | GC | 13 | 67 |
| | targeted | | acid | | | | | |
| 24232 | non- | At4g18880 | linolenic | ARA_LEAF | p-PcUBI | GC | 11 | 18 |
| | targeted | | acid | | | | | |
| 68849 | non- | At4g26080 | linolenic | ARA_LEAF | p-PcUBI | GC | 14 | 32 |
| | targeted | | acid | | | | | |
| 4102 | non- | At4g33040 | linolenic | ARA_LEAF | p-PcUBI | GC | 15 | 43 |
| | targeted | | acid | | | | | |
| 24438 | non- | At5g03720 | linolenic | ARA_LEAF | p-PcUBI | GC | 13 | 35 |
| | targeted | | acid | | | | | |
| 4904 | non- | At5g18600 | linolenic | ARA_LEAF | p-PcUBI | GC | 13 | 35 |
| | targeted | | acid | | | | | |
| 5318 | non- | At5g57050 | linolenic | ARA_LEAF | p-PcUBI | GC | 15 | 40 |
| | targeted | | acid | | | | | |
| 25498 | non- | At5g59220 | linolenic | ARA_LEAF | p-PcUBI | GC | 24 | 60 |
| | targeted | | acid | | | | | |
| 26196 | non- | AvinDRAFT_1624 | linolenic | ARA_LEAF | p-PcUBI | GC | 11 | 26 |
| | targeted | | acid | | | | | |
| 114083 | non- | AvinDRAFT_1788 | linolenic | ARA_LEAF | p-PcUBI | GC | 16 | 26 |
| | targeted | | acid | | | | | |
| 28040 | non- | AvinDRAFT_2754 | linolenic | ARA_LEAF | p-PcUBI | GC | 13 | 26 |
| | targeted | | acid | | | | | |
| 29500 | non- | AvinDRAFT_3253 | linolenic | ARA_LEAF | p-PcUBI | GC | 14 | 58 |
| | targeted | | acid | | | | | |
| 111155 | non- | AvinDRAFT_3577 | linolenic | ARA_LEAF | p-PcUBI | GC | 12 | 43 |
| | targeted | | acid | | | | | |
| 31026 | non- | AvinDRAFT_3605 | linolenic | ARA_LEAF | p-PcUBI | GC | 11 | 46 |
| | targeted | | acid | | | | | |
| 114231 | non- | AvinDRAFT_3629 | linolenic | ARA_LEAF | p-PcUBI | GC | 15 | 47 |
| | targeted | | acid | | | | | |
| 102941 | non- | AvinDRAFT_4847 | linolenic | ARA_LEAF | p-PcUBI | GC | 12 | 26 |
| | targeted | | acid | | | | | |
| 32648 | non- | AvinDRAFT_5467 | linolenic | ARA_LEAF | p-PcUBI | GC | 21 | 54 |
| | targeted | | acid | | | | | |
| 118238 | plastidic | B0195 | linolenic | ARA_LEAF | p-Super | GC | 14 | 31 |
| | | | acid | | | | | |
| 36299 | plastidic | B0828 | linolenic | ARA_SEED_2 | p-USP | GC | 12 | 28 |
| | | | acid | | | | | |
| 76032 | plastidic | B1065 | linolenic | ARA_LEAF | p-Super | GC | 15 | 48 |
| | | | acid | | | | | |
| 118371 | non- | B1093 | linolenic | ARA_LEAF | p-Super | GC | 11 | 25 |
| | targeted | | acid | | | | | |
| 114552 | plastidic | B1095 | linolenic | ARA_LEAF | p-Super | GC | 12 | 47 |
| | | | acid | | | | | |
| 112111 | non- | B1234 | linolenic | ARA_LEAF | p-Super | GC | 11 | 30 |
| | targeted | | acid | | | | | |
| 119135 | non- | B1248 | linolenic | ARA_LEAF | p-Super | GC | 12 | 19 |
| | targeted | | acid | | | | | |
| 37573 | non- | B1280 | linolenic | ARA_LEAF | p-Super | GC | 13 | 28 |
| | targeted | | acid | | | | | |
| 38226 | non- | B1330 | linolenic | ARA_LEAF | p-Super | GC | 15 | 29 |
| | targeted | | acid | | | | | |
| 115056 | non- | B1470 | linolenic | ARA_LEAF | p-Super | GC | 12 | 30 |
| | targeted | | acid | | | | | |
| 38300 | non- | B1597 | linolenic | ARA_LEAF | p-Super | GC | 17 | 33 |
| | targeted | | acid | | | | | |
| 115064 | plastidic | B1611 | linolenic | ARA_LEAF | p-Super | GC | 13 | 43 |
| | | | acid | | | | | |
| 38345 | non- | B1627 | linolenic | ARA_LEAF | p-Super | GC | 11 | 19 |
| | targeted | | acid | | | | | |
| 78753 | non- | B1670 | linolenic | ARA_LEAF | p-Super | GC | 12 | 30 |
| | targeted | | acid | | | | | |
| 78771 | non- | B1672 | linolenic | ARA_LEAF | p-Super | GC | 12 | 26 |
| | targeted | | acid | | | | | |
| 78953 | non- | B1837 | linolenic | ARA_LEAF | p-Super | GC | 14 | 32 |
| | targeted | | acid | | | | | |
| 8920 | non- | B2032 | linolenic | ARA_LEAF | p-Super | GC | 12 | 90 |
| | targeted | | acid | | | | | |
| 39002 | non- | B2107 | linolenic | ARA_LEAF | p-Super | GC | 13 | 31 |
| | targeted | | acid | | | | | |
| 39237 | non- | B2399 | linolenic | ARA_LEAF | p-Super | GC | 30 | 52 |
| | targeted | | acid | | | | | |
| 115628 | non- | B2439 | linolenic | ARA_LEAF | p-Super | GC | 14 | 33 |
| | targeted | | acid | | | | | |
| 40329 | non- | B2474 | linolenic | ARA_LEAF | p-Super | GC | 12 | 38 |
| | targeted | | acid | | | | | |
| 40665 | non- | B2613 | linolenic | ARA_LEAF | p-Super | GC | 13 | 50 |
| | targeted | | acid | | | | | |
| 115655 | non- | B2682 | linolenic | ARA_LEAF | p-Super | GC | 16 | 25 |
| | targeted | | acid | | | | | |
| 115719 | non- | B2790 | linolenic | ARA_LEAF | p-Super | GC | 18 | 31 |
| | targeted | | acid | | | | | |
| 115791 | plastidic | B2836 | linolenic | ARA_LEAF | p-Super | GC | 14 | 38 |
| | | | acid | | | | | |
| 116110 | non- | B3379 | linolenic | ARA_LEAF | p-Super | GC | 11 | 26 |
| | targeted | | acid | | | | | |
| 94380 | non- | B3442 | linolenic | ARA_LEAF | p-Super | GC | 12 | 33 |
| | targeted | | acid | | | | | |
| 119181 | plastidic | B3534 | linolenic | ARA_LEAF | p-Super | GC | 12 | 29 |
| | | | acid | | | | | |
| 10726 | non- | B3823 | linolenic | ARA_LEAF | p-Super | GC | 11 | 29 |
| | targeted | | acid | | | | | |
| 119209 | non- | B4023 | linolenic | ARA_LEAF | p-Super | GC | 13 | 24 |
| | targeted | | acid | | | | | |
| 10811 | non- | GM02LC12622 | linolenic | ARA_LEAF | p-PcUBI | GC | 13 | 42 |
| | targeted | | acid | | | | | |
| 46751 | non- | GM02LC19289 | linolenic | ARA_LEAF | p-PcUBI | GC | 16 | 89 |
| | targeted | | acid | | | | | |
| 94741 | plastidic | Sll0374 | linolenic | ARA_LEAF | p-PcUBI | GC | 15 | 18 |
| | | | acid | | | | | |
| 112661 | mitochondrial | Sll0745 | linolenic | ARA_LEAF | p-PcUBI | GC | 13 | 29 |
| | | | acid | | | | | |
| 52634 | mitochondrial | Sll1031 | linolenic | ARA_LEAF | p-PcUBI | GC | 12 | 46 |
| | | | acid | | | | | |
| 85723 | mitochondrial | Sll1032 | linolenic | ARA_LEAF | p-PcUBI | GC | 12 | 30 |
| | | | acid | | | | | |
| 53878 | mitochondrial | Sll1443 | linolenic | ARA_LEAF | p-PcUBI | GC | 14 | 33 |
| | | | acid | | | | | |
| 95047 | non- | Sll1498 | linolenic | ARA_LEAF | p-PcUBI | GC | 14 | 30 |
| | targeted | | acid | | | | | |
| 86447 | plastidic | Sll1848 | linolenic | ARA_LEAF | p-PcUBI | GC | 17 | 23 |
| | | | acid | | | | | |
| 11471 | mitochondrial | Sll1917 | linolenic | ARA_LEAF | p-PcUBI | GC | 12 | 24 |
| | | | acid | | | | | |
| 55385 | mitochondrial | Sll1920 | linolenic | ARA_LEAF | p-PcUBI | GC | 14 | 34 |
| | | | acid | | | | | |
| 11990 | non- | Slr0338 | linolenic | ARA_LEAF | p-PcUBI | GC | 12 | 25 |
| | targeted | | acid | | | | | |
| 56153 | plastidic | Slr0597 | linolenic | ARA_LEAF | p-PcUBI | GC | 13 | 36 |
| | | | acid | | | | | |
| 119222 | mitochondrial | Slr0949 | linolenic | ARA_LEAF | p-PcUBI | GC | 13 | 26 |
| | | | acid | | | | | |
| 116341 | mitochondrial | Slr1099 | linolenic | ARA_LEAF | p-PcUBI | GC | 12 | 41 |
| | | | acid | | | | | |
| 116460 | mitochondrial | Slr1250 | linolenic | ARA_LEAF | p-PcUBI | GC | 13 | 26 |
| | | | acid | | | | | |
| 58324 | non- | Slr1312 | linolenic | ARA_LEAF | p-PcUBI | GC | 11 | 19 |
| | targeted | | acid | | | | | |
| 12140 | mitochondrial | Slr1791 | linolenic | ARA_LEAF | p-PcUBI | GC | 12 | 25 |
| | | | acid | | | | | |
| 12341 | mitochondrial | Slr2072 | linolenic | ARA_LEAF | p-PcUBI | GC | 18 | 43 |
| | | | acid | | | | | |
| 61553 | non- | TTC0917 | linolenic | ARA_LEAF | p-PcUBI | GC | 15 | 105 |
| | targeted | | acid | | | | | |
| 62079 | non- | TTC1386 | linolenic | ARA_LEAF | p-PcUBI | GC | 16 | 24 |
| | targeted | | acid | | | | | |
| 119347 | plastidic | Ybr092c | linolenic | ARA_LEAF | p-Super | GC | 12 | 25 |
| | | | acid | | | | | |
| 119408 | non- | Ydl022w | linolenic | ARA_SEED_2 | p-PcUBI | GC | 13 | 34 |
| | targeted | | acid | | | | | |
| 120045 | non- | Ydl126c | linolenic | ARA_LEAF | Big35S | GC | 16 | 16 |
| | targeted | | acid | | | | | |
| 63544 | plastidic | Ydr046c | linolenic | ARA_LEAF | p-Super | GC | 17 | 22 |
| | | | acid | | | | | |
| 14706 | plastidic | Ydr131c | linolenic | ARA_LEAF | p-Super | GC | 19 | 38 |
| | | | acid | | | | | |
| 120241 | plastidic | Ydr339c | linolenic | ARA_LEAF | p-Super | GC | 15 | 18 |
| | | | acid | | | | | |
| 90165 | non- | Ydr507c | linolenic | ARA_LEAF | Big35S | GC | 11 | 45 |
| | targeted | | acid | | | | | |
| 120309 | non- | Yfl053w | linolenic | ARA_LEAF | Big35S | GC | 11 | 41 |
| | targeted | | acid | | | | | |
| 120436 | plastidic | Ygl039w | linolenic | ARA_LEAF | p-PcUBI | GC | 17 | 26 |
| | | | acid | | | | | |
| 64964 | plastidic | Yjl073w | linolenic | ARA_LEAF | p-Super | GC | 15 | 27 |
| | | | acid | | | | | |
| 91958 | plastidic | Ymr013c | linolenic | ARA_LEAF | p-Super | GC | 13 | 17 |
| | | | acid | | | | | |
| 117495 | non- | Ynl064c | linolenic | ARA_LEAF | Big35S | GC | 13 | 24 |
| | targeted | | acid | | | | | |
| 118212 | plastidic | Ypl271w | linolenic | ARA_LEAF | p-Super | GC | 13 | 27 |
| | | | acid | | | | | |
| 16883 | plastidic | 59582753_SOYBEAN | linolenic | ARA_LEAF | p-PcUBI | GC | 12 | 54 |
| | | | acid | | | | | |

Column 1 shows the SEQ ID NO, Column 2 shows the expression type (targeted or non-targeted), Column 3 shows the “gene name” (locus), Column 4 shows the metabolite analyzed, Column 5 indicates the A. thaliana source tissue analyzed, Column 6 indicates the used promoter for expression, Column 7 indicates the analytical method. Columns 8 and 9 show the minimum and the maximum increase of the analyzed metabolite (in percent) in comparison to the wild type (=ratio_by_weight, given as percent increase).

When the analyses were repeated independently, all results proved to be significant.

[0505.1.8.9] to [0515.1.8.9] for the disclosure of these paragraphs see [0505.1.8.8] to [0515.1.8.8] above.

In a further embodiment, the present invention relates in paragraphs [0000.1.8.10.] to [0514.1.8.10.] to a further process for the production of the fine chemical stearic acid as defined below and corrresponding embodiments as described herein as follows.

for the disclosure of this paragraph see [0001.1.8.8] above.

Fatty acids and triglycerides have numerous applications in the food and feed industry, in cosmetics and in the drug sector. Depending on whether they are free saturated or unsaturated fatty acids or triglycerides with an increased content of saturated or unsaturated fatty acids, they are suitable for the most varied applications; thus, for example, polyunsaturated fatty acids (=PUFAs) are added to infant formula to increase its nutritional value. The various fatty acids and triglycerides are mainly obtained from microorganisms such as fungi or from oil-producing plants including phytoplankton and algae, such as soybean, oilseed rape, sunflower and others, where they are usually obtained in the form of their triacylglycerides.

Stearic acid (=octadecanoic acid) is one of the many useful types of saturated fatty acids that comes from many animal and vegetable fats and oils. It is a waxy solid that melts at around 70° C. Commenly stearic acid is either prepared by treating animal fat with water at a high pressure and temperature or starting with vegetable oils by hydrogenation of said oils. It is useful as an ingredient in making candles, soaps, and cosmetics and for softening rubber.

Principally microorganisms such as Mortierella or oil producing plants such as soybean, rapeseed or sunflower or algae such as Crytocodinium or Phaeodactylum are a common source for oils containing fatty acids, where they are usually obtained in the form of their triacyl glycerides. Alternatively, they are obtained advantageously from animals, such as fish.

The free fatty acids are prepared advantageously by hydrolysis with a strong base such as potassium or sodium hydroxide.

for the disclosure of this paragraph see [0005.1.8.8] above.

Unlike most saturated fats, stearic acid does not seem to increase cholesterol levels in the blood, because liver enzymes convert it to an unsaturated fat during digestion.

Stearic acid is the most common one of the long-chain fatty acids. It is found in many foods, such as beef fat, and cocoa butter. It is widely used as mentioned above as a lubricant, in soaps, cosmetics, food packaging, deodorant sticks, toothpastes, and as a softener in rubber.

Encouraging research shows that stearic acid, one of the components of the fat found in the cocoa butter of chocolate, may have some positive effects on platelets. The mechanism believed to be responsible for the potential platelet activation by stearic acid involves Arachidonic metabolism, which includes thromboxane A2, a potent aggregating compound, and prostaglandin I2, a potent anti-aggregating compound.

As described above, fatty acids are used in a lot of different applications, for example in cosmetics, pharmaceuticals and in feed and food.

Therefore improving the productivity of such fatty acids and improving the quality of foodstuffs and animal feeds is an important task of the different industries.

To ensure a high productivity of certain fatty acids in plants or microorganism, it is necessary to manipulate the natural biosynthesis of fatty acids in said organism.

Accordingly, there is still a great demand for new and more suitable genes which encode enzymes which participate in the biosynthesis of fatty acids and make it possible to produce certain fatty acids specifically on an industrial scale without unwanted byproducts forming. In the selection of genes for biosynthesis two characteristics above all are particularly important. On the one hand, there is as ever a need for improved processes for obtaining the highest possible contents of fatty acids on the other hand as less as possible byproducts should be produced in the production process. It is an object of the present invention to develop an inexpensive process for the synthesis of stearic acid. Stearic acid is one of the fatty acids , which are most frequently limiting.

for the disclosure of this paragraph see [0013.1.8.8] above.

Accordingly, in a first embodiment, the invention relates to a process for the production of at least one fine chemical selected from the group consisting of: stearic acid, or, in other words, of the “fine chemical” or “fine chemical of the invention”.

The terms “fine chemical of the invention”, “fine chemical” or “the fine chemical” are used herein equally and relate in context of the paragraphs or sections [0014.1.10.10] to [0514.1.8.10] essentially to the metabolite or the metabolites indicated in column 7, application no. 10 of Tables I, II and IV in the respective line.

Further, the term “in context of any of the paragraphs [0014.1.10.10] to [0514.1.8.10]” as used herein means that for any of said paragraphs [0014.1.10.10] to [0514.1.8.10] the term “the fine chemical” is understood to follow the definition of paragraphs or sections [0014.1.10.10] and [0015.1.10.10], independently whether it refers to any other paragraph or not and whether the reference recites the term “fine chemical” in an other context.

Thus, in cases where one or more paragraphs or sections are incorporated by reference into any of the present paragraphs [0014.1.10.10] to [0514.1.8.10], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to the definition of paragraph [0014.1.10.10] and [0015.1.10.10].

Accordingly, in one embodiment, the term “fine chemical of the invention”, “fine chemical” or “the fine chemical” as used herein (alone or in combination, like in FCRP) means “stearic acid in context of the nucleic acid or polypeptide sequences listed in the respective same line of any one of Tables I to IV of application no. 10 and indicating in column 7 the metabolite “stearic acid”.

In one embodiment, the term stearic acid or the term “fine chemical” mean in context of the paragraphs or sections [0014.1.10.10] to [0514.1.8.10] at least one chemical compound with an activity of the above mentioned stearic acid, respectively.

Accordingly, in one embodiment, the terms “fine chemical of the invention”, “fine chemical” or “the fine chemical” mean in context of any of the paragraphs [0014.1.10.10] to [0514.1.8.10] stearic acid, its tryglycerides, lipids, oils and/or fats in free form or bound form. In a preferred embodiment, the term “the fine chemical” means stearic acidin ftree form or e.g. in form of its tryglycerides, lipids, oils and/or fats. In a preferred embodiment “stearic acid” means stearic acid in free form.

In one embodiment, the term “the fine chemical” or “the respective fine chemical” mean in context of any of the paragraphs [0014.1.10.10] to [0514.1.8.10] stearic acid. and/or tryglycerides, lipids, oils and/or fats of stearic acid. and its salts, ester, thioester or stearic acid in free form or bound to other compounds such as triglycerides, glycolipids, phospholipids etc. In a preferred embodiment, the term “the fine chemical” means stearic acid., in free form or its salts or bound to triglycerides. Triglycerides, lipids, oils, fats or lipid mixture thereof shall mean any triglyceride, lipid, oil and/or fat containing any bound or free stearic acid for example sphingolipids, phosphoglycerides, lipids, glycolipids such as glycosphingolipids, phospholipids such as phosphatidylethanolamine, phosphatidylcholine, phosphatidylserine, phosphatidylglycerol, phosphatidylinositol or diphosphatidylglycerol, or as monoacylglyceride, diacylglyceride or triacylglyceride or other fatty acid esters such as acetyl-Coenzym A thioester, which contain further saturated or unsaturated fatty acids in the fatty acid molecule.

In one embodiment, the term “the fine chemical” and the term “the respective fine chemical” mean at least one chemical compound with an activity of the abovementioned fine chemical. Further, the term “in context of any of the paragraphs [0014.1.10.10] to [0514.1.8.10]” as used herein means that for any of said paragraphs [0014.1.10.10] to [0514.1.8.10] the term “the fine chemical” is understood to follow the definition of section [0014.1.10.10] or section [0015.1.10.10], independently whether it refers to any other paragraph or not and whether the reference recites the term “fine chemical” in an other context.

Thus, in cases where one or more paragraphs or sections are incorporated by reference into any of the present paragraphs [0014.1.10.10] to [0514.1.8.10], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to this definition of this paragraph [0015.1.10.10].

Further, the term “fine chemicals” as used herein relates to compositions comprising said fine chemical(s), i.e. comprising stearic acid, respectively.

Accordingly, the present invention relates to a process for the production of stearic acid, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of citrate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of At2g45420-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of F-box protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of CCAAT-binding transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of b0283-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of betaine aldehyde dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of b0371-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of ATP-binding component of a transport system in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of sodium/proton antiporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of lipoprotein precursor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of acid shock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of b1672-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of protease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of b2032-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of b2399-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of ethanolamine utilization protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of NADH dehydrogenase subunit N in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of b2513-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of phospho-2-dehydro-3-deoxyheptonate aldolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of 2-C-methyl-D-erythritol 4-phosphate cytidylyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of b2849-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of aspartate-ammonia ligase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of 3-ketoacyl-CoA thiolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of b3989-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of b4121-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of glucokinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of sll1032-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of CTP synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of coproporphyrinogen III oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of cation-transporting ATPase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of tryptophan synthase alpha chain in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of arginine decarboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of acetyl-coenzyme A synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of 3-deoxy-7-phosphoheptulonate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of ycl049c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of glycerol-3-phosphate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of isocitrate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of ydr507c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of dihydroxyacetone kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of YGR104C-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of aconitate hydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of yor392w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating one or more activities selected from the group consisting of glucose-6-phosphate 1-dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

An embodiment of the present invention relates to a process for the production of stearic acid, which comprises
(a1) increasing or generating the activity of a polypeptide comprising a polypeptide as depicted in a respective line in column 5 or 8 of Table II, application no. 10, or a homolog or a fragment thereof, a consensus sequence or at least one polypeptide motif as depicted in the respective line in column 8 of Table IV, application no. 10; or
(a2) increasing or generating the activity of an expression product of one or more nucleic acid molecule(s) comprising a polynucleotide as depicted in the respective line in column 5 or 8 of Table I, application no. 10, preferably the coding region thereof, or a homolog or a fragment thereof; or
(a3) increasing or generating the activity of a functional equivalent of (a1) or (a2); in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of stearic acid or a composition comprising stearic acid in said non-human organism or in the culture medium surrounding said non-human organism.

Accordingly, the increase or generation of one or more said activities is for example conferred by one or more expression products of said nucleic acid molecule, e.g. proteins. Accordingly, in the present invention described above, the increase or generation of one or more said activities is for example conferred by one or more protein(s) each comprising a polypeptide selected from the group as depicted in Table II, application no. 10, column 5 and 8, or a homolog or a fragment thereof.

The process of the invention comprises in one embodiment the following steps:
(i) increasing or generating of the expression of; and/or
(ii) increasing or generating the expression of an expression product of; and/or
(iii) increasing or generating one or more activities of an expression product encoded by; at least one nucleic acid molecule (in the following “Fine Chemical Related Protein (FCRP)”encoding gene or “FCRP”-gene) comprising a nucleic acid molecule selected from the group consisting of:
(a) a nucleic acid molecule encoding the polypeptide shown in column 5 or 8 of Table II, preferably Table II B, application no. 10, or a homolog or a fragment thereof;
(b) a nucleic acid molecule shown in column 5 or 8 of Table I, preferably Table I B, application no. 10, or a homolog or a fragment thereof (preferably the coding region thereof);
(c) a nucleic acid molecule, which, as a result of the degeneracy of the genetic code, can be derived from a polypeptide sequence depicted in column 5 or 8 of Table II, preferably Table II B, application no. 10;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 10, or the coding region thereof;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d),
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 10;
(i) a nucleic acid molecule encoding a polypeptide having the activity represented by a respective protein as depicted in column 5 of Table II, application no. 10;
(j) a nucleic acid molecule which comprises a polynucleotide, which is obtained by amplifying a cDNA library or a genomic library using the primers as depicted in column 8 of Table III, application no. 10; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

The term “increasing or generating of the expression of” may also be interchanged by the term “overexpressing”.

In a preferred embodiment thereof said nucleic acid molecule encodes a polypeptide which has the activity of the polypeptide represented by a protein comprising a polypeptide as depicted in the corresponding hit in column 5 of Table II, application no. 10.

In another preferred embodiment thereof said nucleic acid molecule confers a generation or an increase of the respective fine chemical in a non-human organism or a part thereof as compared to a corresponding wild-type non-human organism or part thereof.

In another preferred embodiment thereof said nucleic acid molecule encodes a polypeptide which has the activity of the polypeptide represented by a protein as depicted in the corresponding hit in column 5 of Table II, application no. 10, and confers a generation or an increase of the respective fine chemical in a non-human organism or a part thereof as compared to a corresponding wild-type non-human organism or part thereof.

Accordingly, the genes of the present invention or used in accordance with the present invention, which respectively encode a protein having an activity of 2-C-methyl-D-erythritol 4-phosphate cytidylyltransferase, 3-deoxy-7-phosphoheptulonate synthase, 3-ketoacyl-CoA thiolase, acetyl-coenzyme A synthetase, acid shock protein, aconitate hydratase, arginine decarboxylase, aspartate-ammonia ligase, At2g45420-protein, ATP-binding component of a transport system, b0283-protein, b0371-protein, b1672-protein, b2032-protein, b2399-protein, b2513-protein, b2849-protein, b3989-protein, b4121-protein, betaine aldehyde dehydrogenase, calcium-dependent protein kinase, cation-transporting ATPase, CCAAT-binding transcription factor, citrate synthase, coproporphyrinogen III oxidase, CTP synthetase, dihydroxyacetone kinase, ethanolamine utilization protein, F-box protein, glucokinase, glucose-6-phosphate 1-dehydrogenase, glutaredoxin, glycerol-3-phosphate dehydrogenase, heat shock transcription factor, isocitrate dehydrogenase, lipoprotein precursor, monothiol glutaredoxin, NADH dehydrogenase subunit N, oxidoreductase, phospho-2-dehydro-3-deoxyheptonate aldolase, protease, protein phosphatase, pyruvate kinase, sll1032-protein, sodium/proton antiporter, transcription factor, tryptophan synthase alpha chain, ycl049c-protein, ydr507c-protein, YGR104C-protein, or yor392w-protein, which respectively encode a protein comprising a polypeptide encoded by a nucleic acid sequence as shown in Table I, application no. 10, column 5 or 8, (preferably the coding region thereof), or a homolog or a fragment thereof, which respectively encode a protein comprising a polypeptide as depicted in Table II, application no. 10, column 5 or 8, or a homolg or a fragment thereof, and/or which respectively can be amplified with the primer set shown in Table III, application no. 10, column 8, are also referred to as “FCRP genes”.

Proteins or polypeptides encoded by “FCRP-genes” are referred to as “Fine Chemical Related Proteins” or “FCRP”. For the purposes of the description of the present invention, the respective protein having an activity of 2-C-methyl-D-erythritol 4-phosphate cytidylyltransferase, 3-deoxy-7-phosphoheptulonate synthase, 3-ketoacyl-CoA thiolase, acetylcoenzyme A synthetase, acid shock protein, aconitate hydratase, arginine decarboxylase, aspartate-ammonia ligase, At2g45420-protein, ATP-binding component of a transport system, b0283-protein, b0371-protein, b1672-protein, b2032-protein, b2399-protein, b2513-protein, b2849-protein, b3989-protein, b4121-protein, betaine aldehyde dehydrogenase, calcium-dependent protein kinase, cation-transporting ATPase, CCAAT-binding transcription factor, citrate synthase, coproporphyrinogen III oxidase, CTP synthetase, dihydroxyacetone kinase, ethanolamine utilization protein, F-box protein, glucokinase, glucose-6-phosphate 1-dehydrogenase, glutaredoxin, glycerol-3-phosphate dehydrogenase, heat shock transcription factor, isocitrate dehydrogenase, lipoprotein precursor, monothiol glutaredoxin, NADH dehydrogenase subunit N, oxidoreductase, phospho-2-dehydro-3-deoxyheptonate aldolase, protease, protein phosphatase, pyruvate kinase, sll1032-protein, sodium/proton antiporter, transcription factor, tryptophan synthase alpha chain, ycl049c-protein, ydr507c-protein, YGR104C-protein, or yor392w-protein, the respective protein comprising a polypeptide encoded by one or more respective nucleic acid sequences as shown in Table I, application no. 10, column 5 or 8, (preferably the coding region thereof), or a homolog or fragment thereof, the respective protein comprising a respective polypeptide as depicted in Table II, application no. 10, column 5 or 8, or a homolog or fragment thereof, the respective protein comprising a sequence corresponding to the consensus sequence as shown in Table IV, application no. 10, column 8, and/or the respective protein comprising at least one polypeptide motif as shown in Table IV, application no. 10, column 8 is also referred to as Fine Chemical Related Protein” or “FCRP”.

Thus, in one embodiment, the present invention provides a process of the production of stearic acid, by increasing or generating one or more activities, especially selected from the group consisting of 2-C-methyl-D-erythritol 4-phosphate cytidylyltransferase, 3-deoxy-7-phosphoheptulonate synthase, 3-ketoacyl-CoA thiolase, acetyl-coenzyme A synthetase, acid shock protein, aconitate hydratase, arginine decarboxylase, aspartate-ammonia ligase, At2g45420-protein, ATP-binding component of a transport system, b0283-protein, b0371-protein, b1672-protein, b2032-protein, b2399-protein, b2513-protein, b2849-protein, b3989-protein, b4121-protein, betaine aldehyde dehydrogenase, calcium-dependent protein kinase, cation-transporting ATPase, CCAAT-binding transcription factor, citrate synthase, coproporphyrinogen III oxidase, CTP synthetase, dihydroxyacetone kinase, ethanolamine utilization protein, F-box protein, glucokinase, glucose-6-phosphate 1-dehydrogenase, glutaredoxin, glycerol-3-phosphate dehydrogenase, heat shock transcription factor, isocitrate dehydrogenase, lipoprotein precursor, monothiol glutaredoxin, NADH dehydrogenase subunit N, oxidoreductase, phospho-2-dehydro-3-deoxyheptonate aldolase, protease, protein phosphatase, pyruvate kinase, sll1032-protein, sodium/proton antiporter, transcription factor, tryptophan synthase alpha chain, ycl049c-protein, ydr507c-protein, YGR104C-protein, and yor392w-protein, which is conferred by one or more FCRPs or the gene product of one or more FCRP-genes, for example by the gene product of a nucleic acid sequences comprising a polynucleotide selected from the group as shown in Table I, application no. 10, column 5 or 8, (preferably by the coding region thereof), or a homolog or a fragment thereof, e.g. or by one or more proteins each comprising a polypeptide encoded by one or more nucleic acid sequences selected from the group as shown in Table I, application no. 10, column 5 or 8, (preferably by the coding region thereof), or a homolog or a fragment thereof, or by one or more protein(s) each comprising a polypeptide selected from the group as depicted in Table II, application no. 10, column 5 and 8, or a homolog thereof, or a protein comprising a sequence corresponding to the consensus sequence or comprising at least one polypeptide motif as shown in Table IV, application no. 10, column 8.

for the disclosure of this paragraph see [0025.1.8.8] above.

In an embodiment, the process comprises increasing or generating the activity of one or more polypeptides having said activity, e.g. by generating or increasing the amount and/or specific activity in the cell or a compartment of a cell of one of more FCRP, especially selected from the group consisting of 2-C-methyl-D-erythritol 4-phosphate cytidylyltransferase, 3-deoxy-7-phosphoheptulonate synthase, 3-ketoacyl-CoA thiolase, acetyl-coenzyme A synthetase, acid shock protein, aconitate hydratase, arginine decarboxylase, aspartate-ammonia ligase, At2g45420-protein, ATP-binding component of a transport system, b0283-protein, b0371-protein, b1672-protein, b2032-protein, b2399-protein, b2513-protein, b2849-protein, b3989-protein, b4121-protein, betaine aldehyde dehydrogenase, calcium-dependent protein kinase, cation-transporting ATPase, CCAAT-binding transcription factor, citrate synthase, coproporphyrinogen III oxidase, CTP synthetase, dihydroxyacetone kinase, ethanolamine utilization protein, F-box protein, glucokinase, glucose-6-phosphate 1-dehydrogenase, glutaredoxin, glycerol-3-phosphate dehydrogenase, heat shock transcription factor, isocitrate dehydrogenase, lipoprotein precursor, monothiol glutaredoxin, NADH dehydrogenase subunit N, oxidoreductase, phospho-2-dehydro-3-deoxyheptonate aldolase, protease, protein phosphatase, pyruvate kinase, sll1032-protein, sodium/proton antiporter, transcription factor, tryptophan synthase alpha chain, ycl049c-protein, ydr507c-protein, YGR104C-protein, and yor392w-protein, for example of the respective polypeptide as depicted in Table II, application no. 10, column 5 and 8, or a homolog or a fragment thereof, or the respective polypeptide comprising a sequence corresponding to the consensus sequences as shown in Table IV, application no. 10, column 8, or the respective polypeptide comprising at least one polypeptide motif as depicted in Table IV, application no. 10, column 8.

A further embodiment of the present invention relates to a process for the production of stearic acid, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a citrate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a At2g45420-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a F-box protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a CCAAT-binding transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a b0283-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a betaine aldehyde dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a b0371-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a ATP-binding component of a transport system non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a sodium/proton antiporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a lipoprotein precursor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a acid shock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a b1672-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a protease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a b2032-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a b2399-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a ethanolamine utilization protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a NADH dehydrogenase subunit N non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a b2513-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a phospho-2-dehydro-3-deoxyheptonate aldolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a 2-C-methyl-D-erythritol 4-phosphate cytidylyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a b2849-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a aspartate-ammonia ligase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a 3-ketoacyl-CoA thiolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a b3989-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a b4121-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a glucokinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a sll1032-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a CTP synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a coproporphyrinogen III oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a cation-transporting ATPase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a tryptophan synthase alpha chain non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a arginine decarboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a acetyl-coenzyme A synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a 3-deoxy-7-phosphoheptulonate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a ycl049c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a glycerol-3-phosphate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a isocitrate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a ydr507c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a dihydroxyacetone kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a YGR104C-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a aconitate hydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a yor392w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a glucose-6-phosphate 1-dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating the activity of a polypeptide comprising a polypeptide as depicted in the respective line in column 5 or 8 of Table II, application no. 10, or a homolog or a fragment thereof, a consensus sequence or at least one polypeptide motif as depicted in the respective line in column 8 of Table IV, application no. 10; or increasing or generating the activity of an expression product of one or more nucleic acid molecule(s) comprising a polynucleotide as depicted in the respective line in column 5 or 8 of Table I, application no. 10, preferably the coding region thereof, or a homolog or a fragment thereof; non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of stearic acid, or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of citrate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of At2g45420-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of F-box protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of CCAAT-binding transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of b0283-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of betaine aldehyde dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of b0371-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of ATP-binding component of a transport system in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of sodium/proton antiporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of lipoprotein precursor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of acid shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of b1672-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of protease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of b2032-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of b2399-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of ethanolamine utilization protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of NADH dehydrogenase subunit N in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of b2513-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of phospho-2-dehydro-3-deoxyheptonate aldolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of 2-C-methyl-D-erythritol 4-phosphate cytidylyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of b2849-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of aspartate-ammonia ligase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of 3-ketoacyl-CoA thiolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of b3989-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of b4121-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of glucokinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of sll1032-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of CTP synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of coproporphyrinogen III oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of cation-transporting ATPase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of tryptophan synthase alpha chain in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of arginine decarboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of acetyl-coenzyme A synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of 3-deoxy-7-phosphoheptulonate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of ycl049c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of glycerol-3-phosphate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of isocitrate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of ydr507c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of dihydroxyacetone kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of YGR104C-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of aconitate hydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of yor392w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a) increasing or generating one or more activities selected from the group consisting of glucose-6-phosphate 1-dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

A further embodiment of the present invention relates to a process for the production of stearic acid, which comprises
(a1) increasing or generating the activity of a polypeptide comprising a polypeptide as depicted in the respective line in column 5 or 8 of Table II, application no. 10, or a homolog or fragment thereof, a consensus sequence or at least one polypeptide motif as depicted in column 8 of Table IV, application no. 10; or increasing or generating the activity of an expression product of one or more nucleic acid molecule(s) comprising a polynucleotide as depicted in the respective line in column 5 or 8 of Table I, application no. 10, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 10, or a homolog or a fragment thereof, a consensus sequence or at least one polypeptide motif as depicted in the respective line in column 8 of Table IV, application no. 10, which is joined to a transit peptide; or
increasing or generating the activity of an expression product of one or more nucleic acid molecule(s) comprising a polynucleotide as depicted in the respective line in column 5 or 8 of Table I, application no. 10, preferably the coding region thereof, or a homolog or a fragment thereof, which is joined to a nucleic acid sequence encoding an organelle localization sequence, preferably a plastid or a mitochondrion localization sequence, especially a plastid localization sequence;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 10, or a homolog or a fragment thereof, a consensus sequence or at least one polypeptide motif as depicted in the respective line in column 8 of Table IV, application no. 10; or increasing or generating the activity of an expression product of one or more nucleic acid molecule(s) comprising a polynucleotide as depicted in the respective line in column 5 or 8 of Table I, application no. 10, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of stearic acid, or a composition comprising stearic acid in said non-human organism or in the culture medium surrounding said non-human organism.

Accordingly, the present invention relates to a process for the production of stearic acid, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a citrate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a At2g45420-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a F-box protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a CCAAT-binding transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a b0283-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a betaine aldehyde dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a b0371-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a ATP-binding component of a transport system in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a sodium/proton antiporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a lipoprotein precursor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a acid shock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a b1672-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a protease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a b2032-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a b2399-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a ethanolamine utilization protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a NADH dehydrogenase subunit N in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a b2513-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a phospho-2-dehydro-3-deoxyheptonate aldolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a 2-C-methyl-D-erythritol 4-phosphate cytidylyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a b2849-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a aspartate-ammonia ligase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a 3-ketoacyl-CoA thiolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a b3989-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a b4121-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a glucokinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a sll1032-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a CTP synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a coproporphyrinogen III oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a cation-transporting ATPase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a tryptophan synthase alpha chain in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a arginine decarboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a acetyl-coenzyme A synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a 3-deoxy-7-phosphoheptulonate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a ycl049c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a glycerol-3-phosphate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a isocitrate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a ydr507c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a dihydroxyacetone kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a YGR104C-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a aconitate hydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a yor392w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a glucose-6-phosphate 1-dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Accordingly, the present invention relates to a process for the production of stearic acid, which comprises
(a) increasing or generating the activity of a polypeptide comprising a polypeptide as depicted in the respective line in column 5 or 8 of Table II, application no. 10, or a homolog or a fragment thereof, a consensus sequence or at least one polypeptide motif as depicted in the respective line in column 8 of Table IV, application no. 10; or
increasing or generating the activity of an expression product of one or more nucleic acid molecule(s) comprising a polynucleotide as depicted in the respective line in column 5 or 8 of Table I, application no. 10, preferably the coding region thereof, or a homolog or a fragment thereof;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of stearic acid, or a composition comprising stearic acid in said non-human organism or in the culture medium surrounding said non-human organism.

In a further embodiment the activity of the polypeptide comprising a polypeptide as depicted in the respective line in column 5 or 8 of Table II, application no. 10, or a homolog or a fragment thereof, a consensus sequence or at least one polypeptide motif as depicted in the respective line in column 8 of Table IV, application no. 10, is increased or generated non-targeted in the above-mentioned process in a microorganism or plant or a part thereof.

In a further embodiment said polypeptide has the activity of the respective polypeptide represented by a protein comprising a polypeptide as depicted in the respective line in column 5 of Table II, application no. 10.

In a further embodiment the activity of the expression product of one or more nucleic acid molecule(s) comprising a polynucleotide as depicted in the respective line in column 5 or 8 of Table I, application no. 10, preferably the coding region thereof, or a homolog or a fragment thereof, is increased or generated non-targeted in the above-mentioned process in a microorganism or plant or a part thereof.

In a further embodiment the activity of the polypeptide comprising a polypeptide as depicted in the respective line in column 5 or 8 of Table II, application no. 10, or a homolog or a fragment thereof, a consensus sequence or at least one polypeptide motif as depicted in the respective line in column 8 of Table IV, application no. 10, is increased or generated in the above-mentioned process in an organelle preferably in plastids or mitochondria, especially in plastids, of a microorganism or plant.

In a further embodiment said polypeptide has the activity of the respective polypeptide represented by a protein comprising a polypeptide as depicted in the respective line in column 5 of Table II, application no. 10.

In a further embodiment the activity of the expression product of one or more nucleic acid molecule(s) comprising a polynucleotide as depicted in the respective line in column 5 or 8 of Table I, application no. 10, preferably the coding region thereof, or a homolog or a fragment thereof, is increased or generated in the above-mentioned process in an organelle, preferably in plasids or mitochondria, especially in plastids, of a microorganism or plant.

In a further embodiment the activity of the polypeptide comprising a polypeptide as depicted in the respective line in column 5 or 8 of Table II, application no. 10, or a homolog or a fragment thereof, a consensus sequence or at least one polypeptide motif as depicted in the respective line in column 8 of Table IV, application no. 10, is increased or generated in the above-mentioned process in the cyctosol of a cell, of a microorganism or plant.

In a further embodiment said polypeptide has the activity of the respective polypeptide represented by a protein comprising a polypeptide as depicted in the respective line in column 5 of Table II, application no. 10.

In a further embodiment the activity of the expression product of one or more nucleic acid molecule(s) comprising a polynucleotide as depicted in the respective line in column 5 or 8 of Table I, application no. 10, preferably the coding region thereof, or a homolog or a fragment thereof, is increased or generated in the above-mentioned process in the cytosol of a cell, of a microorganism or plant.

[0039.1.8.10] to [0066.1.8.10] for the disclosure of these paragraphs see [0039.1.8.8] to [0066.1.8.8] above.

As mentioned above the nucleic acid sequences coding for the proteins as shown in Table II, application no. 10, column 5 or 8, or homologs or fragments thereof, are joined to a nucleic acid sequence encoding a plastidic transit peptide, e.g, if for the nucleic acid molecule in Table I, column 6 the term “plastidic” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in Table II, application no. 10, column 5 or 8, or homologs or fragments thereof, e.g., if for the nucleic acid molecule in Table I, column 6 the term “plastidic” is indicated.

[0068.1.8.10] to [0072.1.8.10] for the disclosure of these paragraphs see [0068.1.8.8] to [0072.1.8.8] above.

As mentioned above the nucleic acid sequences coding for the proteins as shown in the respective line in Table II, application no. 10, column 5 or 8, or homologs or fragments thereof, are joined to a nucleic acid sequence encoding a mitochondric transit peptide, e.g., if for the respective nucleic acid molecule in Table I, column 6 the term “mitochondrial” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in the respective line in Table II, application no. 10, column 5 or 8, or homologs or fragments thereof, e.g., if for the nucleic acid molecule in the respective line in Table I, column 6 the term “mitochondrial” is indicated. (For th eavoidance ofdoubt, th eterm “mitochondric” and “mitochondrial” are interchangeable.)

[0074.1.8.10] to [0075.1.8.10] for the disclosure of these paragraphs see [0074.1.8.8] to [0075.1.8.8] above.

Nucleic acid sequences coding for the transit peptides may be chemically synthesized either in part or wholly according to structure of transit peptide sequences disclosed in the prior art. Said natural or chemically synthesized sequences can be directly linked to the sequences encoding the mature protein or via a linker nucleic acid sequence, which may be typically less than 500 base pairs, preferably less than 450, 400, 350, 300, 250 or 200 base pairs, more preferably less than 150, 100, 90, 80, 70, 60, 50, 40 or 30 base pairs and most preferably less than 25, 20, 15, 12, 9, 6 or 3 base pairs in length and are in frame to the coding sequence. Furthermore favorable nucleic acid sequences encoding transit peptides may comprise sequences derived from more than one biological and/or chemical source and may include a nucleic acid sequence derived from the amino-terminal region of the mature protein, which in its native state is linked to the transit peptide. In a preferred embodiment of the invention said amino-terminal region of the mature protein is typically less than 150 amino acids, preferably less than 140, 130, 120, 110, 100 or 90 amino acids, more preferably less than 80, 70, 60, 50, 40, 35, 30, 25 or 20 amino acids and most preferably less than 19, 18, 17, 16, 15, 14, 13, 12, 11 or 10 amino acids in length. But even shorter or longer stretches are also possible. In addition target sequences, which facilitate the transport of proteins to other cell compartments such as the vacuole, endoplasmic reticulum, golgi complex, glyoxysomes, peroxisomes, mitochondria (in case of a plastidal transit peptide) or plastids, especially chloroplasts (in case of a mitochondric transit peptide) may be also part of the inventive nucleic acid sequence. The proteins translated from said inventive nucleic acid sequences are a kind of fusion proteins that means the nucleic acid sequences encoding the plastidal transit peptide for example the ones shown in Table a, preferably the last one of the Table a are joint to the respective nucleic acid sequences shown in Table I, application no. 10, columns 5 or 8, especially the coding region thereof, or homologs or fragments thereof, in case of nucleic acid sequences being expressed in plastids, and that means the nucleic acid sequences encoding the mitochondric transit peptide for example the ones shown in Table b, preferably the last one of the Table b are joint to the respective nucleic acid sequences shown in Table I, application no. 10, columns 5 or 8, especially the coding region thereof, or homologs or fragments thereof, in case of nucleic acid sequences being expressed in mitochondria. The person skilled in the art is able to join said sequences in a functional manner. Advantageously the transit peptide part is cleaved off from the protein part shown in Table II, application no. 10, columns 5 or 8, or a homolog or fragment thereof, during or after the transport into the organelles, preferably into the plastids or mitochondria, respectively.

All products of the cleavage of the preferred transit peptide shown in the last line of Table a have preferably the N-terminal amino acid sequences QIA CSS or QIA EFQLTT in front of the start methionine of the respective protein mentioned in Table II, application no. 10, column 5 or 8. or homologs or fragments thereof. Other short amino acid sequences of an range of 1 to 20 amino acids preferable 2 to 15 amino acids, more preferable 3 to 10 amino acids most preferably 4 to 8 amino acids are also possible in front of the start methionine of the protein mentioned in Table II, application no. 10, columns 5 or 8, or homologs or fragments thereof. In case of the amino acid sequence QIA CSS the three amino acids in front of the start methionine are stemming from the LIC (=ligatation independent cloning) cassette. Said short amino acid sequence is preferred in the case of the expression of E. coli genes. In case of the amino acid sequence QIA EFQLTT the six amino acids in front of the start methionine are stemming from the LIC cassette. Said short amino acid sequence is preferred in the case of the expression of S. cerevisiae genes. The skilled worker knowns that other short sequences are also useful in the expression of the respective nucleic acid molecule comprising a polynucleotide as depicted in Table I, application no. 10, column 5 or 8, preferably the coding region thereof, or a homolog or a fragment thereof. Furthermore the skilled worker is aware of the fact that there is not a need for such short sequences in the expression of the genes.

[0077.1.8.10] to [0078.1.8.10] for the disclosure of these paragraphs see [0077.1.8.8] to [0078.1.8.8] above.

Alternatively to the targeting of the respective sequences shown in Table II, application no. 10, columns 5 or 8, or homologs or fragments thereof, preferably of sequences in general encoded in the nucleus with the aid of the targeting sequences mentioned for example in Table a alone, Table b alone, or in combination with other targeting sequences preferably into plastids or mitochondria, respectively, the nucleic acids of the invention can directly be introduced into the plastidal or mitochondrial genome. Therefore in a preferred embodiment the respective nucleic acid sequences shown in the respective line in Table I, application no. 10, columns 5 or 8, preferably the coding region thereof, or homologs or fragments thereof, are directly introduced and expressed in plastids e.g., if for the nucleic acid molecule in the respective line in Table I, column 6, the term “plastidic” is indicated, or are directly introduced and expressed in mitochondria e.g., if for the nucleic acid molecule in the respective line in Table I, column 6 the term “mitochondrial” is indicated.

The term “introduced” in the context of this specification shall mean the insertion of a nucleic acid sequence into the organism by means of a “transfection”, “transduction” or preferably by “transformation”.

A plastid, such as a chloroplast, or a mitochondrion, has been “transformed” by an exogenous (preferably foreign) nucleic acid sequence if a nucleic acid sequence has been introduced into the plastid or mitochondrion that means that this sequence has crossed the membrane or the membranes of the plastid or of the mitochondrion. The exogenous, preferably foreign, DNA may be integrated (covalently linked) into plastid or mitochondrial DNA making up the genome of the plastid or mitochondrion, or it may remain unintegrated (e.g., by including a chloroplast origin of replication). “Stably” integrated DNA sequences are those, which are inherited through plastid or mitochondrion replication, thereby transferring new plastids or mitochondria, with the features of the integrated DNA sequence to the progeny.

[0080.1.8.10] to [0083.1.8.10] for the disclosure of these paragraphs see [0080.1.8.8] to [0083.1.8.8] above.

For the process of the present invention it may be of great advantage that by transforming the plastids the intraspecies specific transgene flow is blocked, because a lot of species such as corn, cotton and rice have a strict maternal inheritance of plastids. By placing the respective genes specified in the respective line in Table I, application no. 10, columns 5 or 8, preferably the coding region, or homologs or fragments thereof, e.g. if for the nucleic acid molecule in the respective line in column 6 of Table I the term “plastidic” is indicated, in the plastids of plants, these genes will not be present in the pollen of said plants.

A further preferred embodiment of the invention relates to the use of so called “chloroplast localization sequences”, in which a first RNA sequence or molecule is capable of transporting or “chaperoning” a second RNA sequence, such as a RNA sequence transcribed from the sequences depicted in the respective line in Table I, application no. 10, columns 5 or 8, preferably the coding region thereof, or a homolog or a fragment thereof, or a sequence encoding a protein, as depicted in the respective line in Table II, application no. 10, columns 5 and 8, or a homolog or a fragment thereof, from an external environment inside a cell or outside a plastid into a chloroplast. In one embodiment the chloroplast localization signal is substantially similar or complementary to a complete or intact viroid sequence. The chloroplast localization signal may be encoded by a DNA sequence, which is transcribed into the chloroplast localization RNA. The term “viroid” refers to a naturally occurring single stranded RNA molecule (Flores, C. R. Acad. Sci. III. 324 (10), 943 (2001)). Viroids usually contain about 200-500 nucleotides and generally exist as circular molecules. Examples of viroids that contain chloroplast localization signals include but are not limited to ASBVd, PLMVd, CChMVd and ELVd. The viroid sequence or a functional part of it can be fused to the sequences depicted in the respective line in Table I, application no. 10, columns 5 or 8, preferably the coding region thereof, or a homolog or a fragment thereof, or a sequence encoding a protein, as depicted in the respective line in Table II, application no. 10, columns 5 or 8, or a homolog or a fragment thereof, in such a manner that the viroid sequence transports a sequence transcribed from a sequence as depicted in the respective line in Table I, application no. 10 columns 5 or 8, preferably the coding region thereof, or a homolog or a fragment thereof, or a sequence encoding a protein as depicted in the respective line in Table II, application no. 10 columns 5 or 8, or a homolog or a fragment thereof, into the chloroplasts, e.g. if for the nucleic acid molecule in column 6 of Table I the term “plastidic” is indicated. A preferred embodiment uses a modified ASBVd (Navarro et al., Virology. 268 (1), 218 (2000)).

In a further specific embodiment the protein to be expressed in the plastids such as the proteins depicted in the respective line in Table II, application no. 10, columns 5 or 8, or a homolog or a fragment thereof, e.g. if for the nucleic acid molecule in the respective line in column 6 of Table I the term “plastidic” is indicated, are encoded by different nucleic acids. Such a method is disclosed in WO 2004/040973, which shall be incorporated by reference. WO 2004/040973 teaches a method, which relates to the translocation of an RNA corresponding to a gene or gene fragment into the chloroplast by means of a chloroplast localization sequence. The genes, which should be expressed in the plant or plant cells, are split into nucleic acid fragments, which are introduced into different compartments in the plant e.g. the nucleus, the plastids and/or mitochondria. Additionally plant cells are described in which the chloroplast contains a ribozyme fused at one end to an RNA encoding a fragment of a protein used in the inventive process such that the ribozyme can trans-splice the translocated fusion RNA to the RNA encoding the gene fragment to form and as the case may be reunite the nucleic acid fragments to an intact mRNA encoding a functional protein for example as disclosed in the respective line in Table II, application no. 10, columns 5 or 8, or a homolog or a fragment thereof.

In a preferred embodiment of the invention the nucleic acid sequences as shown in the respective line in Table I, application no. 10, columns 5 or 8, preferably the coding region thereof, or homologs or fragments thereof, e.g. if for the nucleic acid molecule in the respective line in column 6 of Table I the term “plastidic” is indicated, used in the inventive process are transformed into plastids, which are metabolically active. Those plastids should preferably maintain at a high copy number in the plant or plant tissue of interest, most preferably the chloroplasts found in green plant tissues, such as leaves or cotyledons or in seeds.

For a good expression in the plastids the nucleic acid sequences as shown in the respective line in Table I, application no. 10, columns 5 or 8, preferably the coding region thereof, or homologs or fragments thereof, e.g. if for the nucleic acid molecule in the respective line in column 6 of Table I the term “plastidic” is indicated, are introduced into an expression cassette using preferably a promoter and terminater, which are active in plastids preferably a chloroplast promoter. Examples of such promoters include the psbA promoter from the gene from spinach or pea, the rbcL promoter, and the atpB promoter from corn.

[0089.1.8.10] to [0092.1.8.10] for the disclosure of these paragraphs see [0089.1.8.8] to [0092.1.8.8] above.

Advantageously the process for the production of the fine chemical stearic acid leads to an enhanced production of the respective fine chemical. The terms “enhanced” or “increase” mean at least a 10%, 20%, 30%, 40% or 50%, preferably at least 60%, 70%, 80%, 90% or 100%, more preferably 150%, 200%, 300%, 400% or 500% higher production of the respective fine chemical stearic acid in comparison to the wild-type as defined above, e.g. that means in comparison to a non-human organism without the aforementioned modification of the activity of a protein as shown in the respective line in Table II, application no. 10, column 5 or 8, or a fragment or a homolog thereof. The modification of the activity of a protein as shown in the respective line in Table II, application no. 10, column 5 or 8, or a homolog or a fragment thereof, or their combination can be achieved by joining the protein to a respective transit peptide, e.g. if for the respective encoding nucleic acid molecule in column 6 of Table I the term “plastidic” or “mitochondrial” is indicated.

Surprisingly it was found, that the transgenic expression of a protein as shown in the respective line in Table II, application no. 10, column 5, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, conferred a production of or an increase in stearic acid,respectively, to the transgenic non-human organism as compared to a corresponding non-transformed wild type.

In one embodiment the transgenic expression of a protein as shown in the respective line in Table II, application no. 10, column 5 or 8, or a homolog or a fragment thereof, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, confers a production of or an increase in stearic acid,respectively, to the transgenic non-human organism as compared to a corresponding non-transformed wild type.

Accordingly, in one embodiment, an increase of stearic acid in a non-human organism, as compared to a corresponding non-transformed wild type non-human organism, is conferred in the process of the invention, if the activity of a polypeptide showing the activity of a transcription factor, or if the activity of the polypeptide At1g68320, preferably represented by SEQ ID NO. 1062, or a homolog or fragment thereof, or if the activity of a polypeptide encoded by a nucleic acid molecule comprising the nucleic acid SEQ ID NO. 1061, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 1061 or polypeptide SEQ ID NO. 1062, respectively, or a homolog or a fragment thereof, is increased or generated, or if the activity transcription factor is increased or generated in a non-human organism, like a microorganism or a plant cell, plant or part thereof, especially with non-targeted localization. For example, an increase of the stearic acid of at least 1 percent, particularly in a range of 17 to 84-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a protein phosphatase, or if the activity of the polypeptide At2g25070, preferably represented by SEQ ID NO. 1299, or a homolog or fragment thereof, or if the activity of a polypeptide encoded by a nucleic acid molecule comprising the nucleic acid SEQ ID NO. 1298, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 1298 or polypeptide SEQ ID NO. 1299, respectively, or a homolog or a fragment thereof, is increased or generated, or if the activity protein phosphatase is increased or generated in a non-human organism, like a microorganism or a plant cell, plant or part thereof, especially with non-targeted localization. For example, an increase of the stearic acid of at least 1 percent, particularly in a range of 18 to 44-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a citrate synthase, or if the activity of the polypeptide At2g44350, preferably represented by SEQ ID NO. 120811, or a homolog or fragment thereof, or if the activity of a polypeptide encoded by a nucleic acid molecule comprising the nucleic acid SEQ ID NO. 120810, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 120810 or polypeptide SEQ ID NO. 120811, respectively, or a homolog or a fragment thereof, is increased or generated, or if the activity citrate synthase is increased or generated in a non-human organism, like a microorganism or a plant cell, plant or part thereof, especially with plastidic localization. For example, an increase of the stearic acid of at least 1 percent, particularly in a range of 17 to 25-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a At2g45420-protein, or if the activity of the polypeptide At2g45420, preferably represented by SEQ ID NO. 1624, or a homolog or fragment thereof, or if the activity of a polypeptide encoded by a nucleic acid molecule comprising the nucleic acid SEQ ID NO. 1623, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 1623 or polypeptide SEQ ID NO. 1624, respectively, or a homolog or a fragment thereof, is increased or generated, or if the activity At2g45420-protein is increased or generated in a non-human organism, like a microorganism or a plant cell, plant or part thereof, especially with non-targeted localization. For example, an increase of the stearic acid of at least 1 percent, particularly in a range of 17 to 691-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a F-box protein, or if the activity of the polypeptide At3g16820, preferably represented by SEQ ID NO. 120953, or a homolog or fragment thereof, or if the activity of a polypeptide encoded by a nucleic acid molecule comprising the nucleic acid SEQ ID NO. 120952, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 120952 or polypeptide SEQ ID NO. 120953, respectively, or a homolog or a fragment thereof, is increased or generated, or if the activity F-box protein is increased or generated in a non-human organism, like a microorganism or a plant cell, plant or part thereof, especially with non-targeted localization. For example, an increase of the stearic acid of at least 1 percent, particularly in a range of 20 to 28-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a CCAAT-binding transcription factor, or if the activity of the polypeptide At3g20910, preferably represented by SEQ ID NO. 22922, or a homolog or fragment thereof, or if the activity of a polypeptide encoded by a nucleic acid molecule comprising the nucleic acid SEQ ID NO. 22921, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 22921 or polypeptide SEQ ID NO. 22922, respectively, or a homolog or a fragment thereof, is increased or generated, or if the activity CCAAT-binding transcription factor is increased or generated in a non-human organism, like a microorganism or a plant cell, plant or part thereof, especially with non-targeted localization. For example, an increase of the stearic acid of at least 1 percent, partitularly in a range of 28 to 60-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization. For example, an increase of the stearic acid of at least 1 percent, particularly in a range of 23 to 58-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization. For example, an increase of the stearic acid of at least 1 percent, particularly in a range of 19 to 79-percent is conferred as compared to a corresponding non-transformed wild type non-human organism

Accordingly, in one embodiment, an increase of stearic acid in a non-human organism, as compared to a corresponding non-transformed wild type non-human organism, is conferred in the process of the invention, if the activity of a polypeptide showing the activity of a monothiol glutaredoxin, or if the activity of the polypeptide At4g15670, preferably represented by SEQ ID NO. 3280, or a homolog or fragment thereof, or if the activity of a polypeptide encoded by a nucleic acid molecule comprising the nucleic acid SEQ ID NO. 3279, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 3279 or polypeptide SEQ ID NO. 3280, respectively, or a homolog or a fragment thereof, is increased or generated, or if the activity monothiol glutaredoxin is increased or generated in a non-human organism, like a microorganism or a plant cell, plant or part thereof, especially with non-targeted localization. For example, an increase of the stearic acid of at least 1 percent, particularly in a range of 15 to 89-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a heat shock transcription factor, or if the activity of the polypeptide At4g18880, preferably represented by SEQ ID NO. 24233, or a homolog or fragment thereof, or if the activity of a polypeptide encoded by a nucleic acid molecule comprising the nucleic acid SEQ ID NO. 24232, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 24232 or polypeptide SEQ ID NO. 24233, respectively, or a homolog or a fragment thereof, is increased or generated, or if the activity heat shock transcription factor is increased or generated in a non-human organism, like a microorganism or a plant cell, plant or part thereof, especially with non-targeted localization. For example, an increase of the stearic acid of at least 1 percent, particularly in a range of 20 to 28-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a protein phosphatase, or if the activity of the polypeptide At4g26080, preferably represented by SEQ ID NO. 68850, or a homolog or fragment thereof, or if the activity of a polypeptide encoded by a nucleic acid molecule comprising the nucleic acid SEQ ID NO. 68849, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 68849 or polypeptide SEQ ID NO. 68850, respectively, or a homolog or a fragment thereof, is increased or generated, or if the activity protein phosphatase is increased or generated in a non-human organism, like a microorganism or a plant cell, plant or part thereof, especially with non-targeted localization. For example, an increase of the stearic acid of at least 1 percent, particularly in a range of 17 to 20-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a glutaredoxin, or if the activity of the polypeptide At4g33040, preferably represented by SEQ ID NO. 4103, or a homolog or fragment thereof, or if the activity of a polypeptide encoded by a nucleic acid molecule comprising the nucleic acid SEQ ID NO. 4102, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 4102 or polypeptide SEQ ID NO. 4103, respectively, or a homolog or a fragment thereof, is increased or generated, or if the activity glutaredoxin is increased or generated in a non-human organism, like a microorganism or a plant cell, plant or part thereof, especially with non-targeted localization. For example, an increase of the stearic acid of at least 1 percent, particularly in a range of 21 to 40-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization. For example, an increase of the stearic acid of at least 1 percent, particularly in a range of 99 to 132-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a protein phosphatase, or if the activity of the polypeptide At5g59220, preferably represented by SEQ ID NO. 25499, or a homolog or fragment thereof, or if the activity of a polypeptide encoded by a nucleic acid molecule comprising the nucleic acid SEQ ID NO. 25498, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 25498 or polypeptide SEQ ID NO. 25499, respectively, or a homolog or a fragment thereof, is increased or generated, or if the activity protein phosphatase is increased or generated in a non-human organism, like a microorganism or a plant cell, plant or part thereof, especially with non-targeted localization. For example, an increase of the stearic acid of at least 1 percent, particularly in a range of 15 to 38-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a b0283-protein, or if the activity of the polypeptide B0283, preferably represented by SEQ ID NO. 121077, or a homolog or fragment thereof, or if the activity of a polypeptide encoded by a nucleic acid molecule comprising the nucleic acid SEQ ID NO. 121076, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 121076 or polypeptide SEQ ID NO. 121077, respectively, or a homolog or a fragment thereof, is increased or generated, or if the activity b0283-protein is increased or generated in a non-human organism, like a microorganism or a plant cell, plant or part thereof, especially with plastidic localization. For example, an increase of the stearic acid of at least 1 percent, particularly in a range of 19 to 29-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a betaine aldehyde dehydrogenase, or if the activity of the polypeptide B0312, preferably represented by SEQ ID NO. 121112, or a homolog or fragment thereof, or if the activity of a polypeptide encoded by a nucleic acid molecule comprising the nucleic acid SEQ ID NO. 121111, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 121111 or polypeptide SEQ ID NO. 121112, respectively, or a homolog or a fragment thereof, is increased or generated, or if the activity betaine aldehyde dehydrogenase is increased or generated in a non-human organism, like a microorganism or a plant cell, plant or part thereof, especially with non-targeted localization. For example, an increase of the stearic acid of at least 1 percent, particularly in a range of 12 to 21-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a b0371-protein, or if the activity of the polypeptide B0371, preferably represented by SEQ ID NO. 121638, or a homolog or fragment thereof, or if the activity of a polypeptide encoded by a nucleic acid molecule comprising the nucleic acid SEQ ID NO. 121637, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 121637 or polypeptide SEQ ID NO. 121638, respectively, or a homolog or a fragment thereof, is increased or generated, or if the activity b0371-protein is increased or generated in a non-human organism, like a microorganism or a plant cell, plant or part thereof, especially with non-targeted localization. For example, an increase of the stearic acid of at least 1 percent, particularly in a range of 26 to 60-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a ATP-binding component of a transport system, or if the activity of the polypeptide B0449, preferably represented by SEQ ID NO. 7334, or a homolog or fragment thereof, or if the activity of a polypeptide encoded by a nucleic acid molecule comprising the nucleic acid SEQ ID NO. 7333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 7333 or polypeptide SEQ ID NO. 7334, respectively, or a homolog or a fragment thereof, is increased or generated, or if the activity ATP-binding component of a transport system is increased or generated in a non-human organism, like a microorganism or a plant cell, plant or part thereof, especially with non-targeted localization. For example, an increase of the stearic acid of at least 1 percent, particularly in a range of 18 to 75-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a sodium/proton antiporter, or if the activity of the polypeptide B1186, preferably represented by SEQ ID NO. 37401, or a homolog or fragment thereof, or if the activity of a polypeptide encoded by a nucleic acid molecule comprising the nucleic acid SEQ ID NO. 37400, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 37400 or polypeptide SEQ ID NO. 37401, respectively, or a homolog or a fragment thereof, is increased or generated, or if the activity sodium/proton antiporter is increased or generated in a non-human organism, like a microorganism or a plant cell, plant or part thereof, especially with non-targeted localization. For example, an increase of the stearic acid of at least 1 percent, particularly in a range of 15 to 91-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a lipoprotein precursor, or if the activity of the polypeptide B1431, preferably represented by SEQ ID NO. 38267, or a homolog or fragment thereof, or if the activity of a polypeptide encoded by a nucleic acid molecule comprising the nucleic acid SEQ ID NO. 38266, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 38266 or polypeptide SEQ ID NO. 38267, respectively, or a homolog or a fragment thereof, is increased or generated, or if the activity lipoprotein precursor is increased or generated in a non-human organism, like a microorganism or a plant cell, plant or part thereof, especially with plastidic localization. For example, an increase of the stearic acid of at least 1 percent, particularly in a range of 19 to 31-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a acid shock protein, or if the activity of the polypeptide B1597, preferably represented by SEQ ID NO. 38301, or a homolog or fragment thereof, or if the activity of a polypeptide encoded by a nucleic acid molecule comprising the nucleic acid SEQ ID NO. 38300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 38300 or polypeptide SEQ ID NO. 38301, respectively, or a homolog or a fragment thereof, is increased or generated, or if the activity acid shock protein is increased or generated in a non-human organism, like a microorganism or a plant cell, plant or part thereof, especially with non-targeted localization. For example, an increase of the stearic acid of at least 1 percent, particularly in a range of 22 to 178-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a b1672-protein, or if the activity of the polypeptide B1672, preferably represented by SEQ ID NO. 78772, or a homolog or fragment thereof, or if the activity of a polypeptide encoded by a nucleic acid molecule comprising the nucleic acid SEQ ID NO. 78771, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 78771 or polypeptide SEQ ID NO. 78772, respectively, or a homolog or a fragment thereof, is increased or generated, or if the activity b1672-protein is increased or generated in a non-human organism, like a microorganism or a plant cell, plant or part thereof, especially with non-targeted localization. For example, an increase of the stearic acid of at least 1 percent, particularly in a range of 17 to 30-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a protease, or if the activity of the polypeptide B1845, preferably represented by SEQ ID NO. 38574, or a homolog or fragment thereof, or if the activity of a polypeptide encoded by a nucleic acid molecule comprising the nucleic acid SEQ ID NO. 38573, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 38573 or polypeptide SEQ ID NO. 38574, respectively, or a homolog or a fragment thereof, is increased or generated, or if the activity protease is increased or generated in a non-human organism, like a microorganism or a plant cell, plant or part thereof, especially with non-targeted localization. For example, an increase of the stearic acid of at least 1 percent, particularly in a range of 22 to 31-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a b2032-protein, or if the activity of the polypeptide B2032, preferably represented by SEQ ID NO. 8921, or a homolog or fragment thereof, or if the activity of a polypeptide encoded by a nucleic acid molecule comprising the nucleic acid SEQ ID NO. 8920, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 8920 or polypeptide SEQ ID NO. 8921, respectively, or a homolog or a fragment thereof, is increased or generated, or if the activity b2032-protein is increased or generated in a non-human organism, like a microorganism or a plant cell, plant or part thereof, especially with non-targeted localization. For example, an increase of the stearic acid of at least 1 percent, particularly in a range of 18 to 89-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a b2399-protein, or if the activity of the polypeptide B2399, preferably represented by SEQ ID NO. 39238, or a homolog or fragment thereof, or if the activity of a polypeptide encoded by a nucleic acid molecule comprising the nucleic acid SEQ ID NO. 39237, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 39237 or polypeptide SEQ ID NO. 39238, respectively, or a homolog or a fragment thereof, is increased or generated, or if the activity b2399-protein is increased or generated in a non-human organism, like a microorganism or a plant cell, plant or part thereof, especially with non-targeted localization. For example, an increase of the stearic acid of at least 1 percent, particularly in a range of 18 to 63-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a ethanolamine utilization protein, or if the activity of the polypeptide B2439, preferably represented by SEQ ID NO. 115629, or a homolog or fragment thereof, or if the activity of a polypeptide encoded by a nucleic acid molecule comprising the nucleic acid SEQ ID NO. 115628, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 115628 or polypeptide SEQ ID NO. 115629, respectively, or a homolog or a fragment thereof, is increased or generated, or if the activity ethanolamine utilization protein is increased or generated in a non-human organism, like a microorganism or a plant cell, plant or part thereof, especially with non-targeted localization. For example, an increase of the stearic acid of at least 1 percent, particularly in a range of 16 to 24-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a NADH dehydrogenase subunit N, or if the activity of the polypeptide B2482, preferably represented by SEQ ID NO. 79510, or a homolog or fragment thereof, or if the activity of a polypeptide encoded by a nucleic acid molecule comprising the nucleic acid SEQ ID NO. 79509, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 79509 or polypeptide SEQ ID NO. 79510, respectively, or a homolog or a fragment thereof, is increased or generated, or if the activity NADH dehydrogenase subunit N is increased or generated in a non-human organism, like a microorganism or a plant cell, plant or part thereof, especially with non-targeted localization. For example, an increase of the stearic acid of at least 1 percent, particularly in a range of 19 to 73-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a b2513-protein, or if the activity of the polypeptide B2513, preferably represented by SEQ ID NO. 9168, or a homolog or fragment thereof, or if the activity of a polypeptide encoded by a nucleic acid molecule comprising the nucleic acid SEQ ID NO. 9167, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 9167 or polypeptide SEQ ID NO. 9168, respectively, or a homolog or a fragment thereof, is increased or generated, or if the activity b2513-protein is increased or generated in a non-human organism, like a microorganism or a plant cell, plant or part thereof, especially with non-targeted localization. For example, an increase of the stearic acid of at least 1 percent, particularly in a range of 16 to 121-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a phospho-2-dehydro-3-deoxyheptonate aldolase, or if the activity of the polypeptide B2601, preferably represented by SEQ ID NO. 121658, or a homolog or fragment thereof, or if the activity of a polypeptide encoded by a nucleic acid molecule comprising the nucleic acid SEQ ID NO. 121657, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 121657 or polypeptide SEQ ID NO. 121658, respectively, or a homolog or a fragment thereof, is increased or generated, or if the activity phospho-2-dehydro-3-deoxyheptonate aldolase is increased or generated in a non-human organism, like a microorganism or a plant cell, plant or part thereof, especially with plastidic localization. For example, an increase of the stearic acid of at least 1 percent, particularly in a range of 11 to 51-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a 2-C-methyl-Derythritol 4-phosphate cytidylyltransferase, or if the activity of the polypeptide B2747, preferably represented by SEQ ID NO. 81076, or a homolog or fragment thereof, or if the activity of a polypeptide encoded by a nucleic acid molecule comprising the nucleic acid SEQ ID NO. 81075, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 81075 or polypeptide SEQ ID NO. 81076, respectively, or a homolog or a fragment thereof, is increased or generated, or if the activity 2-C-methyl-D-erythritol 4-phosphate cytidylyltransferase is increased or generated in a non-human organism, like a microorganism or a plant cell, plant or part thereof, especially with non-targeted localization. For example, an increase of the stearic acid of at least 1 percent, particularly in a range of 18 to 44-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a b2849-protein, or if the activity of the polypeptide B2849, preferably represented by SEQ ID NO. 81981, or a homolog or fragment thereof, or if the activity of a polypeptide encoded by a nucleic acid molecule comprising the nucleic acid SEQ ID NO. 81980, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 81980 or polypeptide SEQ ID NO. 81981, respectively, or a homolog or a fragment thereof, is increased or generated, or if the activity b2849-protein is increased or generated in a non-human organism, like a microorganism or a plant cell, plant or part thereof, especially with non-targeted localization. For example, an increase of the stearic acid of at least 1 percent, particularly in a range of 21 to 129-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a aspartateammonia ligase, or if the activity of the polypeptide B3744, preferably represented by SEQ ID NO. 83658, or a homolog or fragment thereof, or if the activity of a polypeptide encoded by a nucleic acid molecule comprising the nucleic acid SEQ ID NO. 83657, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 83657 or polypeptide SEQ ID NO. 83658, respectively, or a homolog or a fragment thereof, is increased or generated, or if the activity aspartate-ammonia ligase is increased or generated in a non-human organism, like a microorganism or a plant cell, plant or part thereof, especially with plastidic localization. For example, an increase of the stearic acid of at least 1 percent, particularly in a range of 9 to 27-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a 3-ketoacyl-CoA thiolase, or if the activity of the polypeptide B3845, preferably represented by SEQ ID NO. 100676, or a homolog or fragment thereof, or if the activity of a polypeptide encoded by a nucleic acid molecule comprising the nucleic acid SEQ ID NO. 100675, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 100675 or polypeptide SEQ ID NO. 100676, respectively, or a homolog or a fragment thereof, is increased or generated, or if the activity 3-ketoacyl-CoA thiolase is increased or generated in a non-human organism, like a microorganism or a plant cell, plant or part thereof, especially with plastidic localization. For example, an increase of the stearic acid of at least 1 percent, particularly in a range of 10 to 27-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a b3989-protein, or if the activity of the polypeptide B3989, preferably represented by SEQ ID NO. 44373, or a homolog or fragment thereof, or if the activity of a polypeptide encoded by a nucleic acid molecule comprising the nucleic acid SEQ ID NO. 44372, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 44372 or polypeptide SEQ ID NO. 44373, respectively, or a homolog or a fragment thereof, is increased or generated, or if the activity b3989-protein is increased or generated in a non-human organism, like a microorganism or a plant cell, plant or part thereof, especially with non-targeted localization. For example, an increase of the stearic acid of at least 1 percent, particularly in a range of 18 to 35-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a b4121-protein, or if the activity of the polypeptide B4121, preferably represented by SEQ ID NO. 44610, or a homolog or fragment thereof, or if the activity of a polypeptide encoded by a nucleic acid molecule comprising the nucleic acid SEQ ID NO. 44609, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 44609 or polypeptide SEQ ID NO. 44610, respectively, or a homolog or a fragment thereof, is increased or generated, or if the activity b4121-protein is increased or generated in a non-human organism, like a microorganism or a plant cell, plant or part thereof, especially with non-targeted localization. For example, an increase of the stearic acid of at least 1 percent, particularly in a range of 18 to 35-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a glutaredoxin, or if the activity of the polypeptide GM02LC12622, preferably represented by SEQ ID NO. 10812, or a homolog or fragment thereof, or if the activity of a polypeptide encoded by a nucleic acid molecule comprising the nucleic acid SEQ ID NO. 10811, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 10811 or polypeptide SEQ ID NO. 10812, respectively, or a homolog or a fragment thereof, is increased or generated, or if the activity glutaredoxin is increased or generated in a non-human organism, like a microorganism or a plant cell, plant or part thereof, especially with non-targeted localization. For example, an increase of the stearic acid of at least 1 percent, particularly in a range of 17 to 99-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a transcription factor, or if the activity of the polypeptide GM02LC19289, preferably represented by SEQ ID NO. 46752, or a homolog or fragment thereof, or if the activity of a polypeptide encoded by a nucleic acid molecule comprising the nucleic acid SEQ ID NO. 46751, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 46751 or polypeptide SEQ ID NO. 46752, respectively, or a homolog or a fragment thereof, is increased or generated, or if the activity transcription factor is increased or generated in a non-human organism, like a microorganism or a plant cell, plant or part thereof, especially with non-targeted localization. For example, an increase of the stearic acid of at least 1 percent, particularly in a range of 17 to 41 percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a glucokinase, or if the activity of the polypeptide Sll0593, preferably represented by SEQ ID NO. 85511, or a homolog or fragment thereof, or if the activity of a polypeptide encoded by a nucleic acid molecule comprising the nucleic acid SEQ ID NO. 85510, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, 11 or IV, application No. 10, column 5 or 8 in the respective same line as the nucleic acid molecule SEQ ID NO. 85510 or polypeptide SEQ ID NO. 85511, respectively, or a homolog or a fragment thereof, is increased or generated, or if the activity glucokinase is increased or generated in a non-human organism, like a microorganism or a plant cell, plant or part thereof, especially with plastidic localization. For example, an increase of the stearic acid of at least 1 percent, particularly in a range of 17 to 54-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a sll1032-protein, or if the activity of the polypeptide Sll1032, preferably represented by SEQ ID NO. 85724, or a homolog or fragment thereof, or if the activity of a polypeptide encoded by a nucleic acid molecule comprising the nucleic acid SEQ ID NO. 85723, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 85723 or polypeptide SEQ ID NO. 85724, respectively, or a homolog or a fragment thereof, is increased or generated, or if the activity sll1032-protein is increased or generated in a non-human organism, like a microorganism or a plant cell, plant or part thereof, especially with mitochondrial localization. For example, an increase of the stearic acid of at least 1 percent, particularly in a range of 17 to 22-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a CTP synthetase, or if the activity of the polypeptide Sll1443, preferably represented by SEQ ID NO. 53879, or a homolog or fragment thereof, or if the activity of a polypeptide encoded by a nucleic acid molecule comprising the nucleic acid SEQ ID NO. 53878, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 53878 or polypeptide SEQ ID NO. 53879, respectively, or a homolog or a fragment thereof, is increased or generated, or if the activity CTP synthetase is increased or generated in a non-human organism, like a microorganism or a plant cell, plant or part thereof, especially with mitochondrial localization. For example, an increase of the stearic acid of at least 1 percent, particularly in a range of 17 to 42-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a coproporphyrinogen III oxidase, or if the activity of the polypeptide Sll1917, preferably represented by SEQ ID NO. 11472, or a homolog or fragment thereof, or if the activity of a polypeptide encoded by a nucleic acid molecule comprising the nucleic acid SEQ ID NO. 11471, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 11471 or polypeptide SEQ ID NO. 11472, respectively, or a homolog or a fragment thereof, is increased or generated, or if the activity coproporphyrinogen III oxidase is increased or generated in a non-human organism, like a microorganism or a plant cell, plant or part thereof, especially with mitochondrial localization. For example, an increase of the stearic acid of at least 1 percent, particularly in a range of 17 to 37-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a cationtransporting ATPase, or if the activity of the polypeptide Sll1920, preferably represented by SEQ ID NO. 55386, or a homolog or fragment thereof, or if the activity of a polypeptide encoded by a nucleic acid molecule comprising the nucleic acid SEQ ID NO. 55385, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 55385 or polypeptide SEQ ID NO. 55386, respectively, or a homolog or a fragment thereof, is increased or generated, or if the activity cation-transporting ATPase is increased or generated in a non-human organism, like a microorganism or a plant cell, plant or part thereof, especially with mitochondrial localization. For example, an increase of the stearic acid of at least 1 percent, particularly in a range of 15 to 34-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a oxidoreductase, or if the activity of the polypeptide Slr0338, preferably represented by SEQ ID NO. 11991, or a homolog or fragment thereof, or if the activity of a polypeptide encoded by a nucleic acid molecule comprising the nucleic acid SEQ ID NO. 11990, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 11990 or polypeptide SEQ ID NO. 11991, respectively, or a homolog or a fragment thereof, is increased or generated, or if the activity oxidoreductase is increased or generated in a non-human organism, like a microorganism or a plant cell, plant or part thereof, especially with plastidic localization. For example, an increase of the stearic acid of at least 1 percent, particularly in a range of 20 to 36-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a tryptophan synthase alpha chain, or if the activity of the polypeptide Slr0966, preferably represented by SEQ ID NO. 87656, or a homolog or fragment thereof, or if the activity of a polypeptide encoded by a nucleic acid molecule comprising the nucleic acid SEQ ID NO. 87655, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 87655 or polypeptide SEQ ID NO. 87656, respectively, or a homolog or a fragment thereof, is increased or generated, or if the activity tryptophan synthase alpha chain is increased or generated in a non-human organism, like a microorganism or a plant cell, plant or part thereof, especially with plastidic localization. For example, an increase of the stearic acid of at least 1 percent, particularly in a range of 27 to 48-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a arginine decarboxylase, or if the activity of the polypeptide Slr1312, preferably represented by SEQ ID NO. 58325, or a homolog or fragment thereof, or if the activity of a polypeptide encoded by a nucleic acid molecule comprising the nucleic acid SEQ ID NO. 58324, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 58324 or polypeptide SEQ ID NO. 58325, respectively, or a homolog or a fragment thereof, is increased or generated, or if the activity arginine decarboxylase is increased or generated in a non-human organism, like a microorganism or a plant cell, plant or part thereof, especially with non-targeted localization. For example, an increase of the stearic acid of at least 1 percent, particularly in a range of 16 to 70-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a acetylcoenzyme A synthetase, or if the activity of the polypeptide Ya1054c, preferably represented by SEQ ID NO. 121909, or a homolog or fragment thereof, or if the activity of a polypeptide encoded by a nucleic acid molecule comprising the nucleic acid SEQ ID NO. 121908, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 121908 or polypeptide SEQ ID NO. 121909, respectively, or a homolog or a fragment thereof, is increased or generated, or if the activity acetyl-coenzyme A synthetase is increased or generated in a non-human organism, like a microorganism or a plant cell, plant or part thereof, especially with non-targeted localization. For example, an increase of the stearic acid of at least 1 percent, particularly in a range of 20 to 28-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a 3-deoxy-7-phosphoheptulonate synthase, or if the activity of the polypeptide Ybr249c, preferably represented by SEQ ID NO. 88880, or a homolog or fragment thereof, or if the activity of a polypeptide encoded by a nucleic acid molecule comprising the nucleic acid SEQ ID NO. 88879, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 88879 or polypeptide SEQ ID NO. 88880, respectively, or a homolog or a fragment thereof, is increased or generated, or if the activity 3-deoxy-7-phosphoheptulonate synthase is increased or generated in a non-human organism, like a microorganism or a plant cell, plant or part thereof, especially with plastidic localization. For example, an increase of the stearic acid of at least 1 percent, particularly in a range of 20 to 109-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a ycl049c-protein, or if the activity of the polypeptide Yc1049c, preferably represented by SEQ ID NO. 113221, or a homolog or fragment thereof, or if the activity of a polypeptide encoded by a nucleic acid molecule comprising the nucleic acid SEQ ID NO. 113220, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 113220 or polypeptide SEQ ID NO. 113221, respectively, or a homolog or a fragment thereof, is increased or generated, or if the activity ycl049c-protein is increased or generated in a non-human organism, like a microorganism or a plant cell, plant or part thereof, especially with plastidic localization. For example, an increase of the stearic acid of at least 1 percent, particularly in a range of 11 to 17-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a glycerol-3-phosphate dehydrogenase, or if the activity of the polypeptide Ydl022w, preferably represented by SEQ ID NO. 119409, or a homolog or fragment thereof, or if the activity of a polypeptide encoded by a nucleic acid molecule comprising the nucleic acid SEQ ID NO. 119408, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 119408 or polypeptide SEQ ID NO. 119409, respectively, or a homolog or a fragment thereof, is increased or generated, or if the activity glycerol-3-phosphate dehydrogenase is increased or generated in a non-human organism, like a microorganism or a plant cell, plant or part thereof, especially with non-targeted localization. For example, an increase of the stearic acid of at least 1 percent, particularly in a range of 17 to 35-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a isocitrate dehydrogenase, or if the activity of the polypeptide Ydl066w, preferably represented by SEQ ID NO. 122229, or a homolog or fragment thereof, or if the activity of a polypeptide encoded by a nucleic acid molecule comprising the nucleic acid SEQ ID NO. 122228, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, 11 or IV, application No. 10, column 5 or 8 in the respective same line as the nucleic acid molecule SEQ ID NO. 122228 or polypeptide SEQ ID NO. 122229, respectively, or a homolog or a fragment thereof, is increased or generated, or if the activity isocitrate dehydrogenase is increased or generated in a non-human organism, like a microorganism or a plant cell, plant or part thereof, especially with non-targeted localization. For example, an increase of the stearic acid of at least 1 percent, particularly in a range of 9 to 24-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a ydr507c-protein, or if the activity of the polypeptide Ydr507c, preferably represented by SEQ ID NO. 90166, or a homolog or fragment thereof, or if the activity of a polypeptide encoded by a nucleic acid molecule comprising the nucleic acid SEQ ID NO. 90165, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 90165 or polypeptide SEQ ID NO. 90166, respectively, or a homolog or a fragment thereof, is increased or generated, or if the activity ydr507c-protein is increased or generated in a non-human organism, like a microorganism or a plant cell, plant or part thereof, especially with non-targeted localization. For example, an increase of the stearic acid of at least 1 percent, particularly in a range of 16 to 51-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a dihydroxyacetone kinase, or if the activity of the polypeptide Yfl053w, preferably represented by SEQ ID NO. 120310, or a homolog or fragment thereof, or if the activity of a polypeptide encoded by a nucleic acid molecule comprising the nucleic acid SEQ ID NO. 120309, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 120309 or polypeptide SEQ ID NO. 120310, respectively, or a homolog or a fragment thereof, is increased or generated, or if the activity dihydroxyacetone kinase is increased or generated in a non-human organism, like a microorganism or a plant cell, plant or part thereof, especially with non-targeted localization. For example, an increase of the stearic acid of at least 1 percent, particularly in a range of 19 to 37-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a YGR104C-protein, or if the activity of the polypeptide Ygr104c, preferably represented by SEQ ID NO. 117483, or a homolog or fragment thereof, or if the activity of a polypeptide encoded by a nucleic acid molecule comprising the nucleic acid SEQ ID NO. 117482, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 117482 or polypeptide SEQ ID NO. 117483, respectively, or a homolog or a fragment thereof, is increased or generated, or if the activity YGR104C-protein is increased or generated in a non-human organism, like a microorganism or a plant cell, plant or part thereof, especially with non-targeted localization. For example, an increase of the stearic acid of at least 1 percent, particularly in a range of 14 to 20-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a aconitate hydratase, or if the activity of the polypeptide Ylr304c, preferably represented by SEQ ID NO. 66773, or a homolog or fragment thereof, or if the activity of a polypeptide encoded by a nucleic acid molecule comprising the nucleic acid SEQ ID NO. 66772, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 66772 or polypeptide SEQ ID NO. 66773, respectively, or a homolog or a fragment thereof, is increased or generated, or if the activity aconitate hydratase is increased or generated in a non-human organism, like a microorganism or a plant cell, plant or part thereof, especially with non-targeted localization. For example, an increase of the stearic acid of at least 1 percent, particularly in a range of 16 to 35-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a yor392w-protein, or if the activity of the polypeptide Yor392w, preferably represented by SEQ ID NO. 15184, or a homolog or fragment thereof, or if the activity of a polypeptide encoded by a nucleic acid molecule comprising the nucleic acid SEQ ID NO. 15183, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 15183 or polypeptide SEQ ID NO. 15184, respectively, or a homolog or a fragment thereof, is increased or generated, or if the activity yor392w-protein is increased or generated in a non-human organism, like a microorganism or a plant cell, plant or part thereof, especially with plastidic localization. For example, an increase of the stearic acid of at least 1 percent, particularly in a range of 20 to 32-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a pyruvate kinase, or if the activity of the polypeptide 51340801_CANOLA, preferably represented by SEQ ID NO. 15533, or a homolog or fragment thereof, or if the activity of a polypeptide encoded by a nucleic acid molecule comprising the nucleic acid SEQ ID NO. 15532, preferably the coding region thereof, or a homolog or fragment thereof, e.g. derived from Brassica napus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 15532 or polypeptide SEQ ID NO. 15533, respectively, or a homolog or a fragment thereof, is increased or generated, or if the activity pyruvate kinase is increased or generated in a non-human organism, like a microorganism or a plant cell, plant or part thereof, especially with plastidic localization. For example, an increase of the stearic acid of at least 1 percent, particularly in a range of 16 to 58-percent is conferred as compared to a corresponding non-transformed wild type non-human organism.

Accordingly, in one embodiment, an increase of stearic acid in a non-human organism, as compared to a corresponding non-transformed wild type non-human organism, is conferred in the process of the invention, if the activity of a polypeptide showing the activity of a glucose-6-phosphate 1-dehydrogenase, or if the activity of the polypeptide 59582753_SOYBEAN, preferably represented by SEQ ID NO. 16884, or a homolog or fragment thereof, or if the activity of a polypeptide encoded by a nucleic acid molecule comprising the nucleic acid SEQ ID NO. 16883, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 10, column 5 or 8 in the respective same line as the nucleic acid molecule SEQ ID NO. 16883 or polypeptide SEQ ID NO. 16884, respectively, or a homolog or a fragment thereof, is increased or generated, or if the activity glucose-6-phosphate 1-dehydrogenase is increased or generated in a non-human organism, like a microorganism or a plant cell, plant or part thereof, especially with plastidic localization. For example, an increase of the stearic acid of at least 1 percent, particularly in a range of 16 to 42-percent is conferred as compared to a corresponding non-transformed wild type non-human organism.

[0096.1.8.10] to [0103.1.8.10] for the disclosure of these paragraphs see [0096.1.8.8] to [0103.1.8.8] above.

In this paragraph “A” means “Goffeau et al., Science 274 (5287), 546 (1996)”, “B” means “Blattner et al., Science 277 (5331), 1453 (1997)”, “C” means “Nakamura et al., Nucleic Acids Res. 28(1), 72 (2000)”, “D” means “deposition of the sequences to the EMBL/GenBank/DDBJ databases under “Sequencing of the draft genome assembly of Azotobacter vinelandii AvOP”. in June 2005 by Copeland et al.”, “E” means “Henne et al., Nat. Biotechnol. 22 (5), 547 (2004)”, “F” means “Kaul et al., Nature 408 (6814), 796 (2000)”, and “G” means “Matsumoto et al., Nature 436 (7052), 793 (2005)”.

The nucleic acid sequence of At1g68320 from Arabidopsis thaliana, e.g. as shown in the respective line in column 5 of Table I, application no. 10, has been published in F. And the activity of the gene product thereof is the activity of transcription factor.

Accordingly, in one embodiment, the process of the present invention for producing stearic acid in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I , application no. 10, preferably the coding region thereof, or a homolog or a fragment thereof, and being depicted in the same respective line as said At1g68320, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said At1g68320, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said At1g68320, or a functional equivalent or a homolog thereof as depicted in column 8 of Table II, application no. 10, preferably a homolog or functional equivalent as depicted in column 8 of Table II B, application no. 10, and being depicted in the same respective line as said At1g68320, and preferably the activity is increased non-targeted.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 1061, preferably the coding region thereof, conferred the production of or the increase in stearic acid compared with the wild type control.

The nucleic acid sequence of At2g25070 from Arabidopsis thaliana, e.g. as shown in the respective line in column 5 of Table I, application no. 10, has been published in F. And the activity of the gene product thereof is the activity of protein phosphatase.

Accordingly, in one embodiment, the process of the present invention for producing stearic acid in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said At2g25070, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said At2g25070, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said At2g25070, or a functional equivalent or a homolog thereof as depicted in column 8 of Table II, application no. 10, preferably a homolog or functional equivalent as depicted in column 8 of Table II B, application no. 10, and being depicted in the same respective line as said At2g25070, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 1298, preferably the coding region thereof, conferred the production of or the increase in stearic acid compared with the wild type control.

The nucleic acid sequence of At2g44350 from Arabidopsis thaliana, e.g. as shown in the respective line in column 5 of Table I, application no. 10, has been published in F. And the activity of the gene product thereof is the activity of citrate synthase.

Accordingly, in one embodiment, the process of the present invention for producing stearic acid in a non-human organism, like a microorganism or a plant or a part thereof, comprises increasing or generating the activity of a gene product with the activity of a gene product conferring the activity of “citrate synthase”, especially from Arabidopsis thaliana or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said At2g44350, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said At2g44350, and preferably the activity is increased plastidic, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said At2g44350, or a functional equivalent or a homolog thereof as depicted in column 8 of Table II, application no. 10, preferably a homolog or functional equivalent as depicted in column 8 of Table II B, application no. 10, and being depicted in the same respective line as said At2g44350, and preferably the activity is increased plastidic.

Accordingly, in one embodiment, the molecule which activity is to be increased in the process of the invention is the gene product with an activity as a “citrate synthase”, preferably it is the molecule of section (a) or (b) of this paragraph.

In particular, it was observed that in plants, especially in Arabidopsis thaliana, increasing or generating the activity of a gene product plastidic with the activity of a “citrate synthase”, preferably being encoded by a gene comprising the nucleic acid sequence SEQ ID NO. 120810, preferably the coding region thereof, conferred the production of or the increase in stearic acid compared with the wild type control.

The nucleic acid sequence of At2g45420 from Arabidopsis thaliana, e.g. as shown in the respective line in column 5 of Table I, application no. 10, has been published in F. And the activity of the gene product thereof is the activity of At2g45420-protein.

Accordingly, in one embodiment, the process of the present invention for producing stearic acid in a non-human organism, like a microorganism or a plant or a part thereof, comprises increasing or generating the activity of a gene product with the activity of a gene product conferring the activity of “At2g45420-protein”, especially from Arabidopsis thaliana or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said At2g45420, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said At2g45420, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said At2g45420, or a functional equivalent or a homolog thereof as depicted in column 8 of Table II, application no. 10, preferably a homolog or functional equivalent as depicted in column 8 of Table II B, application no. 10, and being depicted in the same respective line as said At2g45420, and preferably the activity is increased non-targeted.

Accordingly, in one embodiment, the molecule which activity is to be increased in the process of the invention is the gene product with an activity as a “At2g45420-protein”, preferably it is the molecule of section (a) or (b) of this paragraph.

In particular, it was observed that in plants, especially in Arabidopsis thaliana, increasing or generating the activity of a gene product non-targeted with the activity of a “At2g45420-protein”, preferably being encoded by a gene comprising the nucleic acid sequence SEQ ID NO. 1623, preferably the coding region thereof, conferred the production of or the increase in stearic acid compared with the wild type control.

The nucleic acid sequence of At3g16820 from Arabidopsis thaliana, e.g. as shown in the respective line in column 5 of Table I, application no. 10, has been published in F. And the activity of the gene product thereof is the activity of F-box protein.

Accordingly, in one embodiment, the process of the present invention for producing stearic acid in a non-human organism, like a microorganism or a plant or a part thereof, comprises increasing or generating the activity of a gene product with the activity of a gene product conferring the activity of “F-box protein”, especially from Arabidopsis thaliana or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said At3g16820, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said At3g16820, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said At3g16820, or a functional equivalent or a homolog thereof as depicted in column 8 of Table II, application no. 10, preferably a homolog or functional equivalent as depicted in column 8 of Table II B, application no. 10, and being depicted in the same respective line as said At3g16820, and preferably the activity is increased non-targeted.

Accordingly, in one embodiment, the molecule which activity is to be increased in the process of the invention is the gene product with an activity as a “F-box protein”, preferably it is the molecule of section (a) or (b) of this paragraph.

In particular, it was observed that in plants, especially in Arabidopsis thaliana, increasing or generating the activity of a gene product non-targeted with the activity of a “F-box protein”, preferably being encoded by a gene comprising the nucleic acid sequence SEQ ID NO. 120952, preferably the coding region thereof, conferred the production of or the increase in stearic acid compared with the wild type control.

The nucleic acid sequence of At3g20910 from Arabidopsis thaliana, e.g. as shown in the respective line in column 5 of Table I, application no. 10, has been published in F. And the activity of the gene product thereof is the activity of CCAAT-binding transcription factor.

Accordingly, in one embodiment, the process of the present invention for producing stearic acid in a non-human organism, like a microorganism or a plant or a part thereof, comprises increasing or generating the activity of a gene product with the activity of a gene product conferring the activity of “CCAAT-binding transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said At3g20910, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said At3g20910, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said At3g20910, or a functional equivalent or a homolog thereof as depicted in column 8 of Table II, application no. 10, preferably a homolog or functional equivalent as depicted in column 8 of Table II B, application no. 10, and being depicted in the same respective line as said At3g20910, and preferably the activity is increased non-targeted.

Accordingly, in one embodiment, the molecule which activity is to be increased in the process of the invention is the gene product with an activity as a “CCAAT-binding transcription factor”, preferably it is the molecule of section (a) or (b) of this paragraph.

In particular, it was observed that in plants, especially in Arabidopsis thaliana, increasing or generating the activity of a gene product non-targeted with the activity of a “CCAAT-binding transcription factor”, preferably being encoded by a gene comprising the nucleic acid sequence SEQ ID NO. 22921, preferably the coding region thereof, conferred the production of or the increase in stearic acid compared with the wild type control.

The nucleic acid sequence of At3g62950 from Arabidopsis thaliana, e.g. as shown in the respective line in column 5 of Table I, application no. 10, has been published in F. And the activity of the gene product thereof is the activity of glutaredoxin.

Accordingly, in one embodiment, the process of the present invention for producing stearic acid in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said At3g62950, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said At3g62950, or a functional equivalent or a homolog thereof as depicted in column 8 of Table II, application no. 10, preferably a homolog or functional equivalent as depicted in column 8 of Table II B, application no. 10, and being depicted in the same respective line as said At3g62950, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in stearic acid compared with the wild type control.

The nucleic acid sequence of At4g15660 from Arabidopsis thaliana, e.g. as shown in the respective line in column 5 of Table I, application no. 10, has been published in F. And the activity of the gene product thereof is the activity of monothiol glutaredoxin.

Accordingly, in one embodiment, the process of the present invention for producing stearic acid in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said At4g15660, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said At4g15660, or a functional equivalent or a homolog thereof as depicted in column 8 of Table II, application no. 10, preferably a homolog or functional equivalent as depicted in column 8 of Table II B, application no. 10, and being depicted in the same respective line as said At4g15660, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in stearic acid compared with the wild type control.

The nucleic acid sequence of At4g15670 from Arabidopsis thaliana, e.g. as shown in the respective line in column 5 of Table I, application no. 10, has been published in F. And the activity of the gene product thereof is the activity of monothiol glutaredoxin.

Accordingly, in one embodiment, the process of the present invention for producing stearic acid in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said At4g15670, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said At4g15670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said At4g15670, or a functional equivalent or a homolog thereof as depicted in column 8 of Table II, application no. 10, preferably a homolog or functional equivalent as depicted in column 8 of Table II B, application no. 10, and being depicted in the same respective line as said At4g15670, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3279, preferably the coding region thereof, conferred the production of or the increase in stearic acid compared with the wild type control.

The nucleic acid sequence of At4g18880 from Arabidopsis thaliana, e.g. as shown in the respective line in column 5 of Table I, application no. 10, has been published in F. And the activity of the gene product thereof is the activity of heat shock transcription factor.

Accordingly, in one embodiment, the process of the present invention for producing stearic acid in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said At4g18880, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said At4g18880, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said At4g18880, or a functional equivalent or a homolog thereof as depicted in column 8 of Table II, application no. 10, preferably a homolog or functional equivalent as depicted in column 8 of Table II B, application no. 10, and being depicted in the same respective line as said At4g18880, and preferably the activity is increased non-targeted.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 24232, preferably the coding region thereof, conferred the production of or the increase in stearic acid compared with the wild type control.

The nucleic acid sequence of At4g26080 from Arabidopsis thaliana, e.g. as shown in the respective line in column 5 of Table I, application no. 10, has been published in F. And the activity of the gene product thereof is the activity of protein phosphatase.

Accordingly, in one embodiment, the process of the present invention for producing stearic acid in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said At4g26080, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said At4g26080, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said At4g26080, or a functional equivalent or a homolog thereof as depicted in column 8 of Table II, application no. 10, preferably a homolog or functional equivalent as depicted in column 8 of Table II B, application no. 10, and being depicted in the same respective line as said At4g26080, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 68849, preferably the coding region thereof, conferred the production of or the increase in stearic acid compared with the wild type control.

The nucleic acid sequence of At4g33040 from Arabidopsis thaliana, e.g. as shown in the respective line in column 5 of Table I, application no. 10, has been published in F. And the activity of the gene product thereof is the activity of glutaredoxin.

Accordingly, in one embodiment, the process of the present invention for producing stearic acid in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said At4g33040, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said At4g33040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said At4g33040, or a functional equivalent or a homolog thereof as depicted in column 8 of Table II, application no. 10, preferably a homolog or functional equivalent as depicted in column 8 of Table II B, application no. 10, and being depicted in the same respective line as said At4g33040, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 4102, preferably the coding region thereof, conferred the production of or the increase in stearic acid compared with the wild type control.

The nucleic acid sequence of At4g35310 from Arabidopsis thaliana, e.g. as shown in the respective line in column 5 of Table I, application no. 10, has been published in F. And the activity of the gene product thereof is the activity of calcium-dependent protein kinase.

Accordingly, in one embodiment, the process of the present invention for producing stearic acid in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said At4g35310, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said At4g35310, or a functional equivalent or a homolog thereof as depicted in column 8 of Table II, application no. 10, preferably a homolog or functional equivalent as depicted in column 8 of Table II B, application no. 10, and being depicted in the same respective line as said At4g35310, and preferably the activity is increased non-targeted.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in stearic acid compared with the wild type control.

The nucleic acid sequence of At5g59220 from Arabidopsis thaliana, e.g. as shown in the respective line in column 5 of Table I, application no. 10, has been published in F. And the activity of the gene product thereof is the activity of protein phosphatase.

Accordingly, in one embodiment, the process of the present invention for producing stearic acid in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said At5g59220, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said At5g59220, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said At5g59220, or a functional equivalent or a homolog thereof as depicted in column 8 of Table II, application no. 10, preferably a homolog or functional equivalent as depicted in column 8 of Table II B, application no. 10, and being depicted in the same respective line as said At5g59220, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 25498, preferably the coding region thereof, conferred the production of or the increase in stearic acid compared with the wild type control.

The nucleic acid sequence of B0283 from Escherichia coli, e.g. as shown in the respective line in column 5 of Table I, application no. 10, has been published in B. And the activity of the gene product thereof is the activity of b0283-protein.

Accordingly, in one embodiment, the process of the present invention for producing stearic acid in a non-human organism, like a microorganism or a plant or a part thereof, comprises increasing or generating the activity of a gene product with the activity of a gene product conferring the activity of “b0283-protein”, especially from Escherichia coli or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said B0283, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said B0283, and preferably the activity is increased plastidic, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said B0283, or a functional equivalent or a homolog thereof as depicted in column 8 of Table II, application no. 10, preferably a homolog or functional equivalent as depicted in column 8 of Table II B, application no. 10, and being depicted in the same respective line as said B0283, and preferably the activity is increased plastidic.

Accordingly, in one embodiment, the molecule which activity is to be increased in the process of the invention is the gene product with an activity as a “b0283-protein”, preferably it is the molecule of section (a) or (b) of this paragraph.

In particular, it was observed that in plants, especially in Arabidopsis thaliana, increasing or generating the activity of a gene product plastidic with the activity of a “b0283-protein”, preferably being encoded by a gene comprising the nucleic acid sequence SEQ ID NO. 121076, preferably the coding region thereof, conferred the production of or the increase in stearic acid compared with the wild type control.

The nucleic acid sequence of B0312 from Escherichia coli, e.g. as shown in the respective line in column 5 of Table I, application no. 10, has been published in B. And the activity of the gene product thereof is the activity of betaine aldehyde dehydrogenase.

Accordingly, in one embodiment, the process of the present invention for producing stearic acid in a non-human organism, like a microorganism or a plant or a part thereof, comprises increasing or generating the activity of a gene product with the activity of a gene product conferring the activity of “betaine aldehyde dehydrogenase”, especially from Escherichia coli or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said B0312, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said B0312, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said B0312, or a functional equivalent or a homolog thereof as depicted in column 8 of Table II, application no. 10, preferably a homolog or functional equivalent as depicted in column 8 of Table II B, application no. 10, and being depicted in the same respective line as said B0312, and preferably the activity is increased non-targeted.

Accordingly, in one embodiment, the molecule which activity is to be increased in the process of the invention is the gene product with an activity as a “betaine aldehyde dehydrogenase”, preferably it is the molecule of section (a) or (b) of this paragraph.

In particular, it was observed that in plants, especially in Arabidopsis thaliana, increasing or generating the activity of a gene product non-targeted with the activity of a “betaine aldehyde dehydrogenase”, preferably being encoded by a gene comprising the nucleic acid sequence SEQ ID NO. 121111, preferably the coding region thereof, conferred the production of or the increase in stearic acid compared with the wild type control.

The nucleic acid sequence of B0371 from Escherichia coli, e.g. as shown in the respective line in column 5 of Table I, application no. 10, has been published in B. And the activity of the gene product thereof is the activity of b0371-protein.

Accordingly, in one embodiment, the process of the present invention for producing stearic acid in a non-human organism, like a microorganism or a plant or a part thereof, comprises increasing or generating the activity of a gene product with the activity of a gene product conferring the activity of “b0371-protein”, especially from Escherichia coli or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said B0371, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said B0371, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said B0371, or a functional equivalent or a homolog thereof as depicted in column 8 of Table II, application no. 10, preferably a homolog or functional equivalent as depicted in column 8 of Table II B, application no. 10, and being depicted in the same respective line as said B0371, and preferably the activity is increased non-targeted.

Accordingly, in one embodiment, the molecule which activity is to be increased in the process of the invention is the gene product with an activity as a “b0371-protein”, preferably it is the molecule of section (a) or (b) of this paragraph.

In particular, it was observed that in plants, especially in Arabidopsis thaliana, increasing or generating the activity of a gene product non-targeted with the activity of a “b0371-protein”, preferably being encoded by a gene comprising the nucleic acid sequence SEQ ID NO. 121637, preferably the coding region thereof, conferred the production of or the increase in stearic acid compared with the wild type control.

The nucleic acid sequence of B0449 from Escherichia coli, e.g. as shown in the respective line in column 5 of Table I, application no. 10, has been published in B. And the activity of the gene product thereof is the activity of ATP-binding component of a transport system.

Accordingly, in one embodiment, the process of the present invention for producing stearic acid in a non-human organism, like a microorganism or a plant or a part thereof, comprises increasing or generating the activity of a gene product with the activity of a gene product conferring the activity of “ATP-binding component of a transport system”, especially from Escherichia coli or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said B0449, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said B0449, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said B0449, or a functional equivalent or a homolog thereof as depicted in column 8 of Table II, application no. 10, preferably a homolog or functional equivalent as depicted in column 8 of Table II B, application no. 10, and being depicted in the same respective line as said B0449, and preferably the activity is increased non-targeted.

Accordingly, in one embodiment, the molecule which activity is to be increased in the process of the invention is the gene product with an activity as a “ATP-binding component of a transport system”, preferably it is the molecule of section (a) or (b) of this paragraph.

In particular, it was observed that in plants, especially in Arabidopsis thaliana, increasing or generating the activity of a gene product non-targeted with the activity of a “ATP-binding component of a transport system”, preferably being encoded by a gene comprising the nucleic acid sequence SEQ ID NO. 7333, preferably the coding region thereof, conferred the production of or the increase in stearic acid compared with the wild type control.

The nucleic acid sequence of B1186 from Escherichia coli, e.g. as shown in the respective line in column 5 of Table I, application no. 10, has been published in B. And the activity of the gene product thereof is the activity of sodium/proton antiporter.

Accordingly, in one embodiment, the process of the present invention for producing stearic acid in a non-human organism, like a microorganism or a plant or a part thereof, comprises increasing or generating the activity of a gene product with the activity of a gene product conferring the activity of “sodium/proton antiporter”, especially from Escherichia coli or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said B1186, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said B1186, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said B1186, or a functional equivalent or a homolog thereof as depicted in column 8 of Table II, application no. 10, preferably a homolog or functional equivalent as depicted in column 8 of Table II B, application no. 10, and being depicted in the same respective line as said B1186, and preferably the activity is increased non-targeted.

Accordingly, in one embodiment, the molecule which activity is to be increased in the process of the invention is the gene product with an activity as a “sodium/proton antiporter”, preferably it is the molecule of section (a) or (b) of this paragraph.

In particular, it was observed that in plants, especially in Arabidopsis thaliana, increasing or generating the activity of a gene product non-targeted with the activity of a “sodium/proton antiporter”, preferably being encoded by a gene comprising the nucleic acid sequence SEQ ID NO. 37400, preferably the coding region thereof, conferred the production of or the increase in stearic acid compared with the wild type control.

The nucleic acid sequence of B1431 from Escherichia coli, e.g. as shown in the respective line in column 5 of Table I, application no. 10, has been published in B. And the activity of the gene product thereof is the activity of lipoprotein precursor.

Accordingly, in one embodiment, the process of the present invention for producing stearic acid in a non-human organism, like a microorganism or a plant or a part thereof, comprises increasing or generating the activity of a gene product with the activity of a gene product conferring the activity of “lipoprotein precursor”, especially from Escherichia coli or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said B1431, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said B1431, and preferably the activity is increased plastidic, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said B1431, or a functional equivalent or a homolog thereof as depicted in column 8 of Table II, application no. 10, preferably a homolog or functional equivalent as depicted in column 8 of Table II B, application no. 10, and being depicted in the same respective line as said B1431, and preferably the activity is increased plastidic.

Accordingly, in one embodiment, the molecule which activity is to be increased in the process of the invention is the gene product with an activity as a “lipoprotein precursor”, preferably it is the molecule of section (a) or (b) of this paragraph.

In particular, it was observed that in plants, especially in Arabidopsis thaliana, increasing or generating the activity of a gene product plastidic with the activity of a “lipoprotein precursor”, preferably being encoded by a gene comprising the nucleic acid sequence SEQ ID NO. 38266, preferably the coding region thereof, conferred the production of or the increase in stearic acid compared with the wild type control.

The nucleic acid sequence of B1597 from Escherichia coli, e.g. as shown in the respective line in column 5 of Table I, application no. 10, has been published in B. And the activity of the gene product thereof is the activity of acid shock protein.

Accordingly, in one embodiment, the process of the present invention for producing stearic acid in a non-human organism, like a microorganism or a plant or a part thereof, comprises increasing or generating the activity of a gene product with the activity of a gene product conferring the activity of “acid shock protein”, especially from Escherichia coli or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said B1597, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said B1597, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said B1597, or a functional equivalent or a homolog thereof as depicted in column 8 of Table II, application no. 10, preferably a homolog or functional equivalent as depicted in column 8 of Table II B, application no. 10, and being depicted in the same respective line as said B1597, and preferably the activity is increased non-targeted.

Accordingly, in one embodiment, the molecule which activity is to be increased in the process of the invention is the gene product with an activity as a “acid shock protein”, preferably it is the molecule of section (a) or (b) of this paragraph.

In particular, it was observed that in plants, especially in Arabidopsis thaliana, increasing or generating the activity of a gene product non-targeted with the activity of a “acid shock protein”, preferably being encoded by a gene comprising the nucleic acid sequence SEQ ID NO. 38300, preferably the coding region thereof, conferred the production of or the increase in stearic acid compared with the wild type control.

The nucleic acid sequence of B1672 from Escherichia coli, e.g. as shown in the respective line in column 5 of Table I, application no. 10, has been published in B. And the activity of the gene product thereof is the activity of b1672-protein.

Accordingly, in one embodiment, the process of the present invention for producing stearic acid in a non-human organism, like a microorganism or a plant or a part thereof, comprises increasing or generating the activity of a gene product with the activity of a gene product conferring the activity of “b1672-protein”, especially from Escherichia coli or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said B1672, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said B1672, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said B1672, or a functional equivalent or a homolog thereof as depicted in column 8 of Table II, application no. 10, preferably a homolog or functional equivalent as depicted in column 8 of Table II B, application no. 10, and being depicted in the same respective line as said B1672, and preferably the activity is increased non-targeted.

Accordingly, in one embodiment, the molecule which activity is to be increased in the process of the invention is the gene product with an activity as a “b1672-protein”, preferably it is the molecule of section (a) or (b) of this paragraph.

In particular, it was observed that in plants, especially in Arabidopsis thaliana, increasing or generating the activity of a gene product non-targeted with the activity of a “b1672-protein”, preferably being encoded by a gene comprising the nucleic acid sequence SEQ ID NO. 78771, preferably the coding region thereof, conferred the production of or the increase in stearic acid compared with the wild type control.

The nucleic acid sequence of B1845 from Escherichia coli, e.g. as shown in the respective line in column 5 of Table I, application no. 10, has been published in B. And the activity of the gene product thereof is the activity of protease.

Accordingly, in one embodiment, the process of the present invention for producing stearic acid in a non-human organism, like a microorganism or a plant or a part thereof, comprises increasing or generating the activity of a gene product with the activity of a gene product conferring the activity of “protease”, especially from Escherichia coli or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said B1845, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said B1845, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said B1845, or a functional equivalent or a homolog thereof as depicted in column 8 of Table II, application no. 10, preferably a homolog or functional equivalent as depicted in column 8 of Table II B, application no. 10, and being depicted in the same respective line as said B1845, and preferably the activity is increased non-targeted.

Accordingly, in one embodiment, the molecule which activity is to be increased in the process of the invention is the gene product with an activity as a “protease”, preferably it is the molecule of section (a) or (b) of this paragraph.

In particular, it was observed that in plants, especially in Arabidopsis thaliana, increasing or generating the activity of a gene product non-targeted with the activity of a “protease”, preferably being encoded by a gene comprising the nucleic acid sequence SEQ ID NO. 38573, preferably the coding region thereof, conferred the production of or the increase in stearic acid compared with the wild type control.

The nucleic acid sequence of B2032 from Escherichia coli, e.g. as shown in the respective line in column 5 of Table I, application no. 10, has been published in B. And the activity of the gene product thereof is the activity of b2032-protein.

Accordingly, in one embodiment, the process of the present invention for producing stearic acid in a non-human organism, like a microorganism or a plant or a part thereof, comprises increasing or generating the activity of a gene product with the activity of a gene product conferring the activity of “b2032-protein”, especially from Escherichia coli or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said B2032, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said B2032, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said B2032, or a functional equivalent or a homolog thereof as depicted in column 8 of Table II, application no. 10, preferably a homolog or functional equivalent as depicted in column 8 of Table II B, application no. 10, and being depicted in the same respective line as said B2032, and preferably the activity is increased non-targeted.

Accordingly, in one embodiment, the molecule which activity is to be increased in the process of the invention is the gene product with an activity as a “b2032-protein”, preferably it is the molecule of section (a) or (b) of this paragraph.

In particular, it was observed that in plants, especially in Arabidopsis thaliana, increasing or generating the activity of a gene product non-targeted with the activity of a “b2032-protein”, preferably being encoded by a gene comprising the nucleic acid sequence SEQ ID NO. 8920, preferably the coding region thereof, conferred the production of or the increase in stearic acid compared with the wild type control.

The nucleic acid sequence of B2399 from Escherichia coli, e.g. as shown in the respective line in column 5 of Table I, application no. 10, has been published in B. And the activity of the gene product thereof is the activity of b2399-protein.

Accordingly, in one embodiment, the process of the present invention for producing stearic acid in a non-human organism, like a microorganism or a plant or a part thereof, comprises increasing or generating the activity of a gene product with the activity of a gene product conferring the activity of “b2399-protein”, especially from Escherichia coli or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said B2399, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said B2399, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said B2399, or a functional equivalent or a homolog thereof as depicted in column 8 of Table II, application no. 10, preferably a homolog or functional equivalent as depicted in column 8 of Table II B, application no. 10, and being depicted in the same respective line as said B2399, and preferably the activity is increased non-targeted.

Accordingly, in one embodiment, the molecule which activity is to be increased in the process of the invention is the gene product with an activity as a “b2399-protein”, preferably it is the molecule of section (a) or (b) of this paragraph.

In particular, it was observed that in plants, especially in Arabidopsis thaliana, increasing or generating the activity of a gene product non-targeted with the activity of a “b2399-protein”, preferably being encoded by a gene comprising the nucleic acid sequence SEQ ID NO. 39237, preferably the coding region thereof, conferred the production of or the increase in stearic acid compared with the wild type control.

The nucleic acid sequence of B2439 from Escherichia coli, e.g. as shown in the respective line in column 5 of Table I, application no. 10, has been published in B. And the activity of the gene product thereof is the activity of ethanolamine utilization protein.

Accordingly, in one embodiment, the process of the present invention for producing stearic acid in a non-human organism, like a microorganism or a plant or a part thereof, comprises increasing or generating the activity of a gene product with the activity of a gene product conferring the activity of “ethanolamine utilization protein”, especially from Escherichia coli or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said B2439, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said B2439, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said B2439, or a functional equivalent or a homolog thereof as depicted in column 8 of Table II, application no. 10, preferably a homolog or functional equivalent as depicted in column 8 of Table II B, application no. 10, and being depicted in the same respective line as said B2439, and preferably the activity is increased non-targeted.

Accordingly, in one embodiment, the molecule which activity is to be increased in the process of the invention is the gene product with an activity as a “ethanolamine utilization protein”, preferably it is the molecule of section (a) or (b) of this paragraph.

In particular, it was observed that in plants, especially in Arabidopsis thaliana, increasing or generating the activity of a gene product non-targeted with the activity of a “ethanolamine utilization protein”, preferably being encoded by a gene comprising the nucleic acid sequence SEQ ID NO. 115628, preferably the coding region thereof, conferred the production of or the increase in stearic acid compared with the wild type control.

The nucleic acid sequence of B2482 from Escherichia coli, e.g. as shown in the respective line in column 5 of Table I, application no. 10, has been published in B. And the activity of the gene product thereof is the activity of NADH dehydrogenase subunit N.

Accordingly, in one embodiment, the process of the present invention for producing stearic acid in a non-human organism, like a microorganism or a plant or a part thereof, comprises increasing or generating the activity of a gene product with the activity of a gene product conferring the activity of “NADH dehydrogenase subunit N”, especially from Escherichia coli or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said B2482, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said B2482, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said B2482, or a functional equivalent or a homolog thereof as depicted in column 8 of Table II, application no. 10, preferably a homolog or functional equivalent as depicted in column 8 of Table II B, application no. 10, and being depicted in the same respective line as said B2482, and preferably the activity is increased non-targeted.

Accordingly, in one embodiment, the molecule which activity is to be increased in the process of the invention is the gene product with an activity as a “NADH dehydrogenase subunit N”, preferably it is the molecule of section (a) or (b) of this paragraph.

In particular, it was observed that in plants, especially in Arabidopsis thaliana, increasing or generating the activity of a gene product non-targeted with the activity of a “NADH dehydrogenase subunit N”, preferably being encoded by a gene comprising the nucleic acid sequence SEQ ID NO. 79509, preferably the coding region thereof, conferred the production of or the increase in stearic acid compared with the wild type control.

The nucleic acid sequence of B2513 from Escherichia coli, e.g. as shown in the respective line in column 5 of Table I, application no. 10, has been published in B. And the activity of the gene product thereof is the activity of b2513-protein.

Accordingly, in one embodiment, the process of the present invention for producing stearic acid in a non-human organism, like a microorganism or a plant or a part thereof, comprises increasing or generating the activity of a gene product with the activity of a gene product conferring the activity of “b2513-protein”, especially from Escherichia coli or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said B2513, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said B2513, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said B2513, or a functional equivalent or a homolog thereof as depicted in column 8 of Table II, application no. 10, preferably a homolog or functional equivalent as depicted in column 8 of Table II B, application no. 10, and being depicted in the same respective line as said B2513, and preferably the activity is increased non-targeted.

Accordingly, in one embodiment, the molecule which activity is to be increased in the process of the invention is the gene product with an activity as a “b2513-protein”, preferably it is the molecule of section (a) or (b) of this paragraph.

In particular, it was observed that in plants, especially in Arabidopsis thaliana, increasing or generating the activity of a gene product non-targeted with the activity of a “b2513-protein”, preferably being encoded by a gene comprising the nucleic acid sequence SEQ ID NO. 9167, preferably the coding region thereof, conferred the production of or the increase in stearic acid compared with the wild type control.

The nucleic acid sequence of B2601 from Escherichia coli, e.g. as shown in the respective line in column 5 of Table I, application no. 10, has been published in B. And the activity of the gene product thereof is the activity of phospho-2-dehydro-3-deoxyheptonate aldolase.

Accordingly, in one embodiment, the process of the present invention for producing stearic acid in a non-human organism, like a microorganism or a plant or a part thereof, comprises increasing or generating the activity of a gene product with the activity of a gene product conferring the activity of “phospho-2-dehydro-3-deoxyheptonate aldolase”, especially from Escherichia coli or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said B2601, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said B2601, and preferably the activity is increased plastidic, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said B2601, or a functional equivalent or a homolog thereof as depicted in column 8 of Table II, application no. 10, preferably a homolog or functional equivalent as depicted in column 8 of Table II B, application no. 10, and being depicted in the same respective line as said B2601, and preferably the activity is increased plastidic.

Accordingly, in one embodiment, the molecule which activity is to be increased in the process of the invention is the gene product with an activity as a “phospho-2-dehydro-3-deoxyheptonate aldolase”, preferably it is the molecule of section (a) or (b) of this paragraph.

In particular, it was observed that in plants, especially in Arabidopsis thaliana, increasing or generating the activity of a gene product plastidic with the activity of a “phospho-2-dehydro-3-deoxyheptonate aldolase”, preferably being encoded by a gene comprising the nucleic acid sequence SEQ ID NO. 121657, preferably the coding region thereof, conferred the production of or the increase in stearic acid compared with the wild type control.

The nucleic acid sequence of B2747 from Escherichia coli, e.g. as shown in the respective line in column 5 of Table I, application no. 10, has been published in B. And the activity of the gene product thereof is the activity of 2-C-methyl-D-erythritol 4-phosphate cytidylyltransferase.

Accordingly, in one embodiment, the process of the present invention for producing stearic acid in a non-human organism, like a microorganism or a plant or a part thereof, comprises increasing or generating the activity of a gene product with the activity of a gene product conferring the activity of “2-C-methyl-D-erythritol 4-phosphate cytidylyltransferase”, especially from Escherichia coli or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said B2747, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said B2747, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said B2747, or a functional equivalent or a homolog thereof as depicted in column 8 of Table II, application no. 10, preferably a homolog or functional equivalent as depicted in column 8 of Table II B, application no. 10, and being depicted in the same respective line as said B2747, and preferably the activity is increased non-targeted.

Accordingly, in one embodiment, the molecule which activity is to be increased in the process of the invention is the gene product with an activity as a “2-C-methyl-D-erythritol 4-phosphate cytidylyltransferase”, preferably it is the molecule of section (a) or (b) of this paragraph.

In particular, it was observed that in plants, especially in Arabidopsis thaliana, increasing or generating the activity of a gene product non-targeted with the activity of a “2-C-methyl-D-erythritol 4-phosphate cytidylyltransferase”, preferably being encoded by a gene comprising the nucleic acid sequence SEQ ID NO. 81075, preferably the coding region thereof, conferred the production of or the increase in stearic acid compared with the wild type control.

The nucleic acid sequence of B2849 from Escherichia coli, e.g. as shown in the respective line in column 5 of Table I, application no. 10, has been published in B. And the activity of the gene product thereof is the activity of b2849-protein.

Accordingly, in one embodiment, the process of the present invention for producing stearic acid in a non-human organism, like a microorganism or a plant or a part thereof, comprises increasing or generating the activity of a gene product with the activity of a gene product conferring the activity of “b2849-protein”, especially from Escherichia coli or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said B2849, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said B2849, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said B2849, or a functional equivalent or a homolog thereof as depicted in column 8 of Table II, application no. 10, preferably a homolog or functional equivalent as depicted in column 8 of Table II B, application no. 10, and being depicted in the same respective line as said B2849, and preferably the activity is increased non-targeted.

Accordingly, in one embodiment, the molecule which activity is to be increased in the process of the invention is the gene product with an activity as a “b2849-protein”, preferably it is the molecule of section (a) or (b) of this paragraph.

In particular, it was observed that in plants, especially in Arabidopsis thaliana, increasing or generating the activity of a gene product non-targeted with the activity of a “b2849-protein”, preferably being encoded by a gene comprising the nucleic acid sequence SEQ ID NO. 81980, preferably the coding region thereof, conferred the production of or the increase in stearic acid compared with the wild type control.

The nucleic acid sequence of B3744 from Escherichia coli, e.g. as shown in the respective line in column 5 of Table I, application no. 10, has been published in B. And the activity of the gene product thereof is the activity of aspartate-ammonia ligase.

Accordingly, in one embodiment, the process of the present invention for producing stearic acid in a non-human organism, like a microorganism or a plant or a part thereof, comprises increasing or generating the activity of a gene product with the activity of a gene product conferring the activity of “aspartate-ammonia ligase”, especially from Escherichia coli or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said B3744, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said B3744, and preferably the activity is increased plastidic, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said B3744, or a functional equivalent or a homolog thereof as depicted in column 8 of Table II, application no. 10, preferably a homolog or functional equivalent as depicted in column 8 of Table II B, application no. 10, and being depicted in the same respective line as said B3744, and preferably the activity is increased plastidic.

Accordingly, in one embodiment, the molecule which activity is to be increased in the process of the invention is the gene product with an activity as a “aspartate-ammonia ligase”, preferably it is the molecule of section (a) or (b) of this paragraph.

In particular, it was observed that in plants, especially in Arabidopsis thaliana, increasing or generating the activity of a gene product plastidic with the activity of a “aspartate-ammonia ligase”, preferably being encoded by a gene comprising the nucleic acid sequence SEQ ID NO. 83657, preferably the coding region thereof, conferred the production of or the increase in stearic acid compared with the wild type control.

The nucleic acid sequence of B3845 from Escherichia coli, e.g. as shown in the respective line in column 5 of Table I, application no. 10, has been published in B. And the activity of the gene product thereof is the activity of 3-ketoacyl-CoA thiolase.

Accordingly, in one embodiment, the process of the present invention for producing stearic acid in a non-human organism, like a microorganism or a plant or a part thereof, comprises increasing or generating the activity of a gene product with the activity of a gene product conferring the activity of “3-ketoacyl-CoA thiolase”, especially from Escherichia coli or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said B3845, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said B3845, and preferably the activity is increased plastidic, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said B3845, or a functional equivalent or a homolog thereof as depicted in column 8 of Table II, application no. 10, preferably a homolog or functional equivalent as depicted in column 8 of Table II B, application no. 10, and being depicted in the same respective line as said B3845, and preferably the activity is increased plastidic.

Accordingly, in one embodiment, the molecule which activity is to be increased in the process of the invention is the gene product with an activity as a “3-ketoacyl-CoA thiolase”, preferably it is the molecule of section (a) or (b) of this paragraph.

In particular, it was observed that in plants, especially in Arabidopsis thaliana, increasing or generating the activity of a gene product plastidic with the activity of a “3-ketoacyl-CoA thiolase”, preferably being encoded by a gene comprising the nucleic acid sequence SEQ ID NO. 100675, preferably the coding region thereof, conferred the production of or the increase in stearic acid compared with the wild type control.

The nucleic acid sequence of B3989 from Escherichia coli, e.g. as shown in the respective line in column 5 of Table I, application no. 10, has been published in B. And the activity of the gene product thereof is the activity of b3989-protein.

Accordingly, in one embodiment, the process of the present invention for producing stearic acid in a non-human organism, like a microorganism or a plant or a part thereof, comprises increasing or generating the activity of a gene product with the activity of a gene product conferring the activity of “b3989-protein”, especially from Escherichia coli or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said B3989, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said B3989, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said B3989, or a functional equivalent or a homolog thereof as depicted in column 8 of Table II, application no. 10, preferably a homolog or functional equivalent as depicted in column 8 of Table II B, application no. 10, and being depicted in the same respective line as said B3989, and preferably the activity is increased non-targeted.

Accordingly, in one embodiment, the molecule which activity is to be increased in the process of the invention is the gene product with an activity as a “b3989-protein”, preferably it is the molecule of section (a) or (b) of this paragraph.

In particular, it was observed that in plants, especially in Arabidopsis thaliana, increasing or generating the activity of a gene product non-targeted with the activity of a “b3989-protein”, preferably being encoded by a gene comprising the nucleic acid sequence SEQ ID NO. 44372, preferably the coding region thereof, conferred the production of or the increase in stearic acid compared with the wild type control.

The nucleic acid sequence of B4121 from Escherichia coli, e.g. as shown in the respective line in column 5 of Table I, application no. 10, has been published in B. And the activity of the gene product thereof is the activity of b4121-protein.

Accordingly, in one embodiment, the process of the present invention for producing stearic acid in a non-human organism, like a microorganism or a plant or a part thereof, comprises increasing or generating the activity of a gene product with the activity of a gene product conferring the activity of “b4121-protein”, especially from Escherichia coli or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said B4121, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said B4121, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said B4121, or a functional equivalent or a homolog thereof as depicted in column 8 of Table II, application no. 10, preferably a homolog or functional equivalent as depicted in column 8 of Table II B, application no. 10, and being depicted in the same respective line as said B4121, and preferably the activity is increased non-targeted.

Accordingly, in one embodiment, the molecule which activity is to be increased in the process of the invention is the gene product with an activity as a “b4121-protein”, preferably it is the molecule of section (a) or (b) of this paragraph.

In particular, it was observed that in plants, especially in Arabidopsis thaliana, increasing or generating the activity of a gene product non-targeted with the activity of a “b4121-protein”, preferably being encoded by a gene comprising the nucleic acid sequence SEQ ID NO. 44609, preferably the coding region thereof, conferred the production of or the increase in stearic acid compared with the wild type control.

The nucleic acid sequence of GM02LC12622 from Glycine max, e.g. as shown in the respective line in column 5 of Table I, application no. 10, is unpublished. And the activity of the gene product thereof is the activity of glutaredoxin.

Accordingly, in one embodiment, the process of the present invention for producing stearic acid in a non-human organism, like a microorganism or a plant or a part thereof, comprises increasing or generating the activity of a gene product with the activity of a gene product conferring the activity of “glutaredoxin”, especially from Glycine max or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said GM02LC12622, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said GM02LC12622, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said GM02LC12622, or a functional equivalent or a homolog thereof as depicted in column 8 of Table II, application no. 10, preferably a homolog or functional equivalent as depicted in column 8 of Table II B, application no. 10, and being depicted in the same respective line as said GM02LC12622, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 10811, preferably the coding region thereof, conferred the production of or the increase in stearic acid compared with the wild type control.

The nucleic acid sequence of GM02LC19289 from Glycine max, e.g. as shown in the respective line in column 5 of Table I, application no. 10, is unpublished. And the activity of the gene product thereof is the activity of transcription factor.

Accordingly, in one embodiment, the process of the present invention for producing stearic acid in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said GM02LC19289, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said GM02LC19289, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said GM02LC19289, or a functional equivalent or a homolog thereof as depicted in column 8 of Table II, application no. 10, preferably a homolog or functional equivalent as depicted in column 8 of Table II B, application no. 10, and being depicted in the same respective line as said GM02LC19289, and preferably the activity is increased non-targeted.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 46751, preferably the coding region thereof, conferred the production of or the increase in stearic acid compared with the wild type control.

The nucleic acid sequence of Sll0593 from Synechocystis sp., e.g. as shown in the respective line in column 5 of Table I, application no. 10, has been published in C. And the activity of the gene product thereof is the activity of glucokinase.

Accordingly, in one embodiment, the process of the present invention for producing stearic acid in a non-human organism, like a microorganism or a plant or a part thereof, comprises increasing or generating the activity of a gene product with the activity of a gene product conferring the activity of “glucokinase”, especially from Synechocystis sp. or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said Sll0593, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said Sll0593, and preferably the activity is increased plastidic, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said Sll0593, or a functional equivalent or a homolog thereof as depicted in column 8 of Table II, application no. 10, preferably a homolog or functional equivalent as depicted in column 8 of Table II B, application no. 10, and being depicted in the same respective line as said Sll0593, and preferably the activity is increased plastidic.

Accordingly, in one embodiment, the molecule which activity is to be increased in the process of the invention is the gene product with an activity as a “glucokinase”, preferably it is the molecule of section (a) or (b) of this paragraph.

In particular, it was observed that in plants, especially in Arabidopsis thaliana, increasing or generating the activity of a gene product plastidic with the activity of a “glucokinase”, preferably being encoded by a gene comprising the nucleic acid sequence SEQ ID NO. 85510, preferably the coding region thereof, conferred the production of or the increase in stearic acid compared with the wild type control.

The nucleic acid sequence of Sll1032 from Synechocystis sp., e.g. as shown in the respective line in column 5 of Table I, application no. 10, has been published in C. And the activity of the gene product thereof is the activity of sll1032-protein.

Accordingly, in one embodiment, the process of the present invention for producing stearic acid in a non-human organism, like a microorganism or a plant or a part thereof, comprises increasing or generating the activity of a gene product with the activity of a gene product conferring the activity of “sll1032-protein”, especially from Synechocystis sp. or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said Sll1032, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said Sll1032, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said Sll1032, or a functional equivalent or a homolog thereof as depicted in column 8 of Table II, application no. 10, preferably a homolog or functional equivalent as depicted in column 8 of Table II B, application no. 10, and being depicted in the same respective line as said Sll1032, and preferably the activity is increased mitochondrial.

Accordingly, in one embodiment, the molecule which activity is to be increased in the process of the invention is the gene product with an activity as a “sll1032-protein”, preferably it is the molecule of section (a) or (b) of this paragraph.

In particular, it was observed that in plants, especially in Arabidopsis thaliana, increasing or generating the activity of a gene product mitochondrial with the activity of a “sll1032-protein”, preferably being encoded by a gene comprising the nucleic acid sequence SEQ ID NO. 85723, preferably the coding region thereof, conferred the production of or the increase in stearic acid compared with the wild type control.

The nucleic acid sequence of Sll1443 from Synechocystis sp., e.g. as shown in the respective line in column 5 of Table I, application no. 10, has been published in C. And the activity of the gene product thereof is the activity of CTP synthetase.

Accordingly, in one embodiment, the process of the present invention for producing stearic acid in a non-human organism, like a microorganism or a plant or a part thereof, comprises increasing or generating the activity of a gene product with the activity of a gene product conferring the activity of “CTP synthetase”, especially from Synechocystis sp. or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said Sll1443, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said Sll1443, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said Sll1443, or a functional equivalent or a homolog thereof as depicted in column 8 of Table II, application no. 10, preferably a homolog or functional equivalent as depicted in column 8 of Table II B, application no. 10, and being depicted in the same respective line as said Sll1443, and preferably the activity is increased mitochondrial.

Accordingly, in one embodiment, the molecule which activity is to be increased in the process of the invention is the gene product with an activity as a “CTP synthetase”, preferably it is the molecule of section (a) or (b) of this paragraph.

In particular, it was observed that in plants, especially in Arabidopsis thaliana, increasing or generating the activity of a gene product mitochondrial with the activity of a “CTP synthetase”, preferably being encoded by a gene comprising the nucleic acid sequence SEQ ID NO. 53878, preferably the coding region thereof, conferred the production of or the increase in stearic acid compared with the wild type control.

The nucleic acid sequence of Sll1917 from Synechocystis sp., e.g. as shown in the respective line in column 5 of Table I, application no. 10, has been published in C. And the activity of the gene product thereof is the activity of coproporphyrinogen III oxidase.

Accordingly, in one embodiment, the process of the present invention for producing stearic acid in a non-human organism, like a microorganism or a plant or a part thereof, comprises increasing or generating the activity of a gene product with the activity of a gene product conferring the activity of “coproporphyrinogen III oxidase”, especially from Synechocystis sp. or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said Sll1917, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said Sll1917, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said Sll1917, or a functional equivalent or a homolog thereof as depicted in column 8 of Table II, application no. 10, preferably a homolog or functional equivalent as depicted in column 8 of Table II B, application no. 10, and being depicted in the same respective line as said Sll1917, and preferably the activity is increased mitochondrial.

Accordingly, in one embodiment, the molecule which activity is to be increased in the process of the invention is the gene product with an activity as a “coproporphyrinogen III oxidase”, preferably it is the molecule of section (a) or (b) of this paragraph.

In particular, it was observed that in plants, especially in Arabidopsis thaliana, increasing or generating the activity of a gene product mitochondrial with the activity of a “coproporphyrinogen III oxidase”, preferably being encoded by a gene comprising the nucleic acid sequence SEQ ID NO. 11471, preferably the coding region thereof, conferred the production of or the increase in stearic acid compared with the wild type control.

The nucleic acid sequence of Sll1920 from Synechocystis sp., e.g. as shown in the respective line in column 5 of Table I, application no. 10, has been published in C. And the activity of the gene product thereof is the activity of cation-transporting ATPase.

Accordingly, in one embodiment, the process of the present invention for producing stearic acid in a non-human organism, like a microorganism or a plant or a part thereof, comprises increasing or generating the activity of a gene product with the activity of a gene product conferring the activity of “cation-transporting ATPase”, especially from Synechocystis sp. or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said Sll1920, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said Sll1920, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said Sll1920, or a functional equivalent or a homolog thereof as depicted in column 8 of Table II, application no. 10, preferably a homolog or functional equivalent as depicted in column 8 of Table II B, application no. 10, and being depicted in the same respective line as said Sll1920, and preferably the activity is increased mitochondrial.

Accordingly, in one embodiment, the molecule which activity is to be increased in the process of the invention is the gene product with an activity as a “cation-transporting ATPase”, preferably it is the molecule of section (a) or (b) of this paragraph.

In particular, it was observed that in plants, especially in Arabidopsis thaliana, increasing or generating the activity of a gene product mitochondrial with the activity of a “cation-transporting ATPase”, preferably being encoded by a gene comprising the nucleic acid sequence SEQ ID NO. 55385, preferably the coding region thereof, conferred the production of or the increase in stearic acid compared with the wild type control.

The nucleic acid sequence of Slr0338 from Synechocystis sp., e.g. as shown in the respective line in column 5 of Table I, application no. 10, has been published in C. And the activity of the gene product thereof is the activity of oxidoreductase.

Accordingly, in one embodiment, the process of the present invention for producing stearic acid in a non-human organism, like a microorganism or a plant or a part thereof, comprises increasing or generating the activity of a gene product with the activity of a gene product conferring the activity of “oxidoreductase”, especially from Synechocystis sp. or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said Slr0338, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said Slr0338, and preferably the activity is increased plastidic, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said Slr0338, or a functional equivalent or a homolog thereof as depicted in column 8 of Table II, application no. 10, preferably a homolog or functional equivalent as depicted in column 8 of Table II B, application no. 10, and being depicted in the same respective line as said Slr0338, and preferably the activity is increased plastidic.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product plastidic with the activity of a “oxidoreductase”, preferably being encoded by a gene comprising the nucleic acid sequence SEQ ID NO. 11990, preferably the coding region thereof, conferred the production of or the increase in stearic acid compared with the wild type control.

The nucleic acid sequence of Slr0966 from Synechocystis sp., e.g. as shown in the respective line in column 5 of Table I, application no. 10, has been published in C. And the activity of the gene product thereof is the activity of tryptophan synthase alpha chain.

Accordingly, in one embodiment, the process of the present invention for producing stearic acid in a non-human organism, like a microorganism or a plant or a part thereof, comprises increasing or generating the activity of a gene product with the activity of a gene product conferring the activity of “tryptophan synthase alpha chain”, especially from Synechocystis sp. or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said Slr0966, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said Slr0966, and preferably the activity is increased plastidic, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said Slr0966, or a functional equivalent or a homolog thereof as depicted in column 8 of Table II, application no. 10, preferably a homolog or functional equivalent as depicted in column 8 of Table II B, application no. 10, and being depicted in the same respective line as said Slr0966, and preferably the activity is increased plastidic.

Accordingly, in one embodiment, the molecule which activity is to be increased in the process of the invention is the gene product with an activity as a “tryptophan synthase alpha chain”, preferably it is the molecule of section (a) or (b) of this paragraph.

In particular, it was observed that in plants, especially in Arabidopsis thaliana, increasing or generating the activity of a gene product plastidic with the activity of a “tryptophan synthase alpha chain”, preferably being encoded by a gene comprising the nucleic acid sequence SEQ ID NO. 87655, preferably the coding region thereof, conferred the production of or the increase in stearic acid compared with the wild type control.

The nucleic acid sequence of Slr1312 from Synechocystis sp., e.g. as shown in the respective line in column 5 of Table I, application no. 10, has been published in C. And the activity of the gene product thereof is the activity of arginine decarboxylase.

Accordingly, in one embodiment, the process of the present invention for producing stearic acid in a non-human organism, like a microorganism or a plant or a part thereof, comprises increasing or generating the activity of a gene product with the activity of a gene product conferring the activity of “arginine decarboxylase”, especially from Synechocystis sp. or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said Slr1312, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said Slr1312, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said Slr1312, or a functional equivalent or a homolog thereof as depicted in column 8 of Table II, application no. 10, preferably a homolog or functional equivalent as depicted in column 8 of Table II B, application no. 10, and being depicted in the same respective line as said Slr1312, and preferably the activity is increased non-targeted.

Accordingly, in one embodiment, the molecule which activity is to be increased in the process of the invention is the gene product with an activity as a “arginine decarboxylase”, preferably it is the molecule of section (a) or (b) of this paragraph.

In particular, it was observed that in plants, especially in Arabidopsis thaliana, increasing or generating the activity of a gene product non-targeted with the activity of a “arginine decarboxylase”, preferably being encoded by a gene comprising the nucleic acid sequence SEQ ID NO. 58324, preferably the coding region thereof, conferred the production of or the increase in stearic acid compared with the wild type control.

The nucleic acid sequence of Yal054c from Saccharomyces cerevisiae, e.g. as shown in the respective line in column 5 of Table I, application no. 10, has been published in A. And the activity of the gene product thereof is the activity of acetyl-coenzyme A synthetase.

Accordingly, in one embodiment, the process of the present invention for producing stearic acid in a non-human organism, like a microorganism or a plant or a part thereof, comprises increasing or generating the activity of a gene product with the activity of a gene product conferring the activity of “acetyl-coenzyme A synthetase”, especially from Saccharomyces cerevisiae or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said Yal054c, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said Yal054c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said Yal054c, or a functional equivalent or a homolog thereof as depicted in column 8 of Table II, application no. 10, preferably a homolog or functional equivalent as depicted in column 8 of Table II B, application no. 10, and being depicted in the same respective line as said Yal054c, and preferably the activity is increased non-targeted.

Accordingly, in one embodiment, the molecule which activity is to be increased in the process of the invention is the gene product with an activity as a “acetyl-coenzyme A synthetase”, preferably it is the molecule of section (a) or (b) of this paragraph.

In particular, it was observed that in plants, especially in Arabidopsis thaliana, increasing or generating the activity of a gene product non-targeted with the activity of a “acetyl-coenzyme A synthetase”, preferably being encoded by a gene comprising the nucleic acid sequence SEQ ID NO. 121908, preferably the coding region thereof, conferred the production of or the increase in stearic acid compared with the wild type control.

The nucleic acid sequence of Ybr249c from Saccharomyces cerevisiae, e.g. as shown in the respective line in column 5 of Table I, application no. 10, has been published in A. And the activity of the gene product thereof is the activity of 3-deoxy-7-phosphoheptulonate synthase.

Accordingly, in one embodiment, the process of the present invention for producing stearic acid in a non-human organism, like a microorganism or a plant or a part thereof, comprises increasing or generating the activity of a gene product with the activity of a gene product conferring the activity of “3-deoxy-7-phosphoheptulonate synthase”, especially from Saccharomyces cerevisiae or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said Ybr249c, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said Ybr249c, and preferably the activity is increased plastidic, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said Ybr249c, or a functional equivalent or a homolog thereof as depicted in column 8 of Table II, application no. 10, preferably a homolog or functional equivalent as depicted in column 8 of Table II B, application no. 10, and being depicted in the same respective line as said Ybr249c, and preferably the activity is increased plastidic.

Accordingly, in one embodiment, the molecule which activity is to be increased in the process of the invention is the gene product with an activity as a “3-deoxy-7-phosphoheptulonate synthase”, preferably it is the molecule of section (a) or (b) of this paragraph.

In particular, it was observed that in plants, especially in Arabidopsis thaliana, increasing or generating the activity of a gene product plastidic with the activity of a “3-deoxy-7-phosphoheptulonate synthase”, preferably being encoded by a gene comprising the nucleic acid sequence SEQ ID NO. 88879, preferably the coding region thereof, conferred the production of or the increase in stearic acid compared with the wild type control.

The nucleic acid sequence of Ycl049c from Saccharomyces cerevisiae, e.g. as shown in the respective line in column 5 of Table I, application no. 10, has been published in A. And the activity of the gene product thereof is the activity of ycl049c-protein.

Accordingly, in one embodiment, the process of the present invention for producing stearic acid in a non-human organism, like a microorganism or a plant or a part thereof, comprises increasing or generating the activity of a gene product with the activity of a gene product conferring the activity of “ycl049c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said Ycl049c, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said Ycl049c, and preferably the activity is increased plastidic, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said Ycl049c, or a functional equivalent or a homolog thereof as depicted in column 8 of Table II, application no. 10, preferably a homolog or functional equivalent as depicted in column 8 of Table II B, application no. 10, and being depicted in the same respective line as said Ycl049c, and preferably the activity is increased plastidic.

Accordingly, in one embodiment, the molecule which activity is to be increased in the process of the invention is the gene product with an activity as a “ycl049c-protein”, preferably it is the molecule of section (a) or (b) of this paragraph.

In particular, it was observed that in plants, especially in Arabidopsis thaliana, increasing or generating the activity of a gene product plastidic with the activity of a “ycl049c-protein”, preferably being encoded by a gene comprising the nucleic acid sequence SEQ ID NO. 113220, preferably the coding region thereof, conferred the production of or the increase in stearic acid compared with the wild type control.

The nucleic acid sequence of Ydl022w from Saccharomyces cerevisiae, e.g. as shown in the respective line in column 5 of Table I, application no. 10, has been published in A. And the activity of the gene product thereof is the activity of glycerol-3-phosphate dehydrogenase.

Accordingly, in one embodiment, the process of the present invention for producing stearic acid in a non-human organism, like a microorganism or a plant or a part thereof, comprises increasing or generating the activity of a gene product with the activity of a gene product conferring the activity of “glycerol-3-phosphate dehydrogenase”, especially from Saccharomyces cerevisiae or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said Ydl022w, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said Ydl022w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said Ydl022w, or a functional equivalent or a homolog thereof as depicted in column 8 of Table II, application no. 10, preferably a homolog or functional equivalent as depicted in column 8 of Table II B, application no. 10, and being depicted in the same respective line as said Ydl022w, and preferably the activity is increased non-targeted.

Accordingly, in one embodiment, the molecule which activity is to be increased in the process of the invention is the gene product with an activity as a “glycerol-3-phosphate dehydrogenase”, preferably it is the molecule of section (a) or (b) of this paragraph.

In particular, it was observed that in plants, especially in Arabidopsis thaliana, increasing or generating the activity of a gene product non-targeted with the activity of a “glycerol-3-phosphate dehydrogenase”, preferably being encoded by a gene comprising the nucleic acid sequence SEQ ID NO. 119408, preferably the coding region thereof, conferred the production of or the increase in stearic acid compared with the wild type control.

The nucleic acid sequence of Ydl066w from Saccharomyces cerevisiae, e.g. as shown in the respective line in column 5 of Table I, application no. 10, has been published in A. And the activity of the gene product thereof is the activity of isocitrate dehydrogenase.

Accordingly, in one embodiment, the process of the present invention for producing stearic acid in a non-human organism, like a microorganism or a plant or a part thereof, comprises increasing or generating the activity of a gene product with the activity of a gene product conferring the activity of “isocitrate dehydrogenase”, especially from Saccharomyces cerevisiae or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said Ydl066w, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said Ydl066w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said Ydl066w, or a functional equivalent or a homolog thereof as depicted in column 8 of Table II, application no. 10, preferably a homolog or functional equivalent as depicted in column 8 of Table II B, application no. 10, and being depicted in the same respective line as said Ydl066w, and preferably the activity is increased non-targeted.

Accordingly, in one embodiment, the molecule which activity is to be increased in the process of the invention is the gene product with an activity as a “isocitrate dehydrogenase”, preferably it is the molecule of section (a) or (b) of this paragraph.

In particular, it was observed that in plants, especially in Arabidopsis thaliana, increasing or generating the activity of a gene product non-targeted with the activity of a “isocitrate dehydrogenase”, preferably being encoded by a gene comprising the nucleic acid sequence SEQ ID NO. 122228, preferably the coding region thereof, conferred the production of or the increase in stearic acid compared with the wild type control.

The nucleic acid sequence of Ydr507c from Saccharomyces cerevisiae, e.g. as shown in the respective line in column 5 of Table I, application no. 10, has been published in A. And the activity of the gene product thereof is the activity of ydr507c-protein.

Accordingly, in one embodiment, the process of the present invention for producing stearic acid in a non-human organism, like a microorganism or a plant or a part thereof, comprises increasing or generating the activity of a gene product with the activity of a gene product conferring the activity of “ydr507c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said Ydr507c, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said Ydr507c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said Ydr507c, or a functional equivalent or a homolog thereof as depicted in column 8 of Table II, application no. 10, preferably a homolog or functional equivalent as depicted in column 8 of Table II B, application no. 10, and being depicted in the same respective line as said Ydr507c, and preferably the activity is increased non-targeted.

Accordingly, in one embodiment, the molecule which activity is to be increased in the process of the invention is the gene product with an activity as a “ydr507c-protein”, preferably it is the molecule of section (a) or (b) of this paragraph.

In particular, it was observed that in plants, especially in Arabidopsis thaliana, increasing or generating the activity of a gene product non-targeted with the activity of a “ydr507c-protein”, preferably being encoded by a gene comprising the nucleic acid sequence SEQ ID NO. 90165, preferably the coding region thereof, conferred the production of or the increase in stearic acid compared with the wild type control.

The nucleic acid sequence of Yfl053w from Saccharomyces cerevisiae, e.g. as shown in the respective line in column 5 of Table I, application no. 10, has been published in A. And the activity of the gene product thereof is the activity of dihydroxyacetone kinase.

Accordingly, in one embodiment, the process of the present invention for producing stearic acid in a non-human organism, like a microorganism or a plant or a part thereof, comprises increasing or generating the activity of a gene product with the activity of a gene product conferring the activity of “dihydroxyacetone kinase”, especially from Saccharomyces cerevisiae or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said Yfl053w, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said Yfl053w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said Yfl053w, or a functional equivalent or a homolog thereof as depicted in column 8 of Table II, application no. 10, preferably a homolog or functional equivalent as depicted in column 8 of Table II B, application no. 10, and being depicted in the same respective line as said Yfl053w, and preferably the activity is increased non-targeted.

Accordingly, in one embodiment, the molecule which activity is to be increased in the process of the invention is the gene product with an activity as a “dihydroxyacetone kinase”, preferably it is the molecule of section (a) or (b) of this paragraph.

In particular, it was observed that in plants, especially in Arabidopsis thaliana, increasing or generating the activity of a gene product non-targeted with the activity of a “dihydroxyacetone kinase”, preferably being encoded by a gene comprising the nucleic acid sequence SEQ ID NO. 120309, preferably the coding region thereof, conferred the production of or the increase in stearic acid compared with the wild type control.

The nucleic acid sequence of Ygr104c from Saccharomyces cerevisiae, e.g. as shown in the respective line in column 5 of Table I, application no. 10, has been published in A. And the activity of the gene product thereof is the activity of YGR104C-protein.

Accordingly, in one embodiment, the process of the present invention for producing stearic acid in a non-human organism, like a microorganism or a plant or a part thereof, comprises increasing or generating the activity of a gene product with the activity of a gene product conferring the activity of “YGR104C-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said Ygr104c, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said Ygr104c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said Ygr104c, or a functional equivalent or a homolog thereof as depicted in column 8 of Table II, application no. 10, preferably a homolog or functional equivalent as depicted in column 8 of Table II B, application no. 10, and being depicted in the same respective line as said Ygr104c, and preferably the activity is increased non-targeted.

Accordingly, in one embodiment, the molecule which activity is to be increased in the process of the invention is the gene product with an activity as a “YGR104C-protein”, preferably it is the molecule of section (a) or (b) of this paragraph.

In particular, it was observed that in plants, especially in Arabidopsis thaliana, increasing or generating the activity of a gene product non-targeted with the activity of a “YGR104C-protein”, preferably being encoded by a gene comprising the nucleic acid sequence SEQ ID NO. 117482, preferably the coding region thereof, conferred the production of or the increase in stearic acid compared with the wild type control.

The nucleic acid sequence of Ylr304c from Saccharomyces cerevisiae, e.g. as shown in the respective line in column 5 of Table I, application no. 10, has been published in A. And the activity of the gene product thereof is the activity of aconitate hydratase.

Accordingly, in one embodiment, the process of the present invention for producing stearic acid in a non-human organism, like a microorganism or a plant or a part thereof, comprises increasing or generating the activity of a gene product with the activity of a gene product conferring the activity of “aconitate hydratase”, especially from Saccharomyces cerevisiae or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said Ylr304c, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said Ylr304c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said Ylr304c, or a functional equivalent or a homolog thereof as depicted in column 8 of Table II, application no. 10, preferably a homolog or functional equivalent as depicted in column 8 of Table II B, application no. 10, and being depicted in the same respective line as said Ylr304c, and preferably the activity is increased non-targeted.

Accordingly, in one embodiment, the molecule which activity is to be increased in the process of the invention is the gene product with an activity as a “aconitate hydratase”, preferably it is the molecule of section (a) or (b) of this paragraph.

In particular, it was observed that in plants, especially in Arabidopsis thaliana, increasing or generating the activity of a gene product non-targeted with the activity of a “aconitate hydratase”, preferably being encoded by a gene comprising the nucleic acid sequence SEQ ID NO. 66772, preferably the coding region thereof, conferred the production of or the increase in stearic acid compared with the wild type control.

The nucleic acid sequence of Yor392w from Saccharomyces cerevisiae, e.g. as shown in the respective line in column 5 of Table I, application no. 10, has been published in A. And the activity of the gene product thereof is the activity of yor392w-protein.

Accordingly, in one embodiment, the process of the present invention for producing stearic acid in a non-human organism, like a microorganism or a plant or a part thereof, comprises increasing or generating the activity of a gene product with the activity of a gene product conferring the activity of “yor392w-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said Yor392w, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said Yor392w, and preferably the activity is increased plastidic, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said Yor392w, or a functional equivalent or a homolog thereof as depicted in column 8 of Table II, application no. 10, preferably a homolog or functional equivalent as depicted in column 8 of Table II B, application no. 10, and being depicted in the same respective line as said Yor392w, and preferably the activity is increased plastidic.

Accordingly, in one embodiment, the molecule which activity is to be increased in the process of the invention is the gene product with an activity as a “yor392w-protein”, preferably it is the molecule of section (a) or (b) of this paragraph.

In particular, it was observed that in plants, especially in Arabidopsis thaliana, increasing or generating the activity of a gene product plastidic with the activity of a “yor392w-protein”, preferably being encoded by a gene comprising the nucleic acid sequence SEQ ID NO. 15183, preferably the coding region thereof, conferred the production of or the increase in stearic acid compared with the wild type control.

The nucleic acid sequence of 51340801_CANOLA from Brassica napus, e.g. as shown in the respective line in column 5 of Table I, application no. 10, is unpublished. And the activity of the gene product thereof is the activity of pyruvate kinase.

Accordingly, in one embodiment, the process of the present invention for producing stearic acid in a non-human organism, like a microorganism or a plant or a part thereof, comprises increasing or generating the activity of a gene product with the activity of a gene product conferring the activity of “pyruvate kinase”, especially from Brassica napus or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said 51340801_CANOLA, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said 51340801_CANOLA, and preferably the activity is increased plastidic, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said 51340801_CANOLA, or a functional equivalent or a homolog thereof as depicted in column 8 of Table II, application no. 10, preferably a homolog or functional equivalent as depicted in column 8 of Table II B, application no. 10, and being depicted in the same respective line as said 51340801_CANOLA, and preferably the activity is increased plastidic.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 15532, preferably the coding region thereof, conferred the production of or the increase in stearic acid compared with the wild type control.

The nucleic acid sequence of 59582753_SOYBEAN from Glycine max, e.g. as shown in the respective line in column 5 of Table I, application no. 10, is unpublished. And the activity of the gene product thereof is the activity of glucose-6-phosphate 1-dehydrogenase.

Accordingly, in one embodiment, the process of the present invention for producing stearic acid in a non-human organism, like a microorganism or a plant or a part thereof, comprises increasing or generating the activity of a gene product with the activity of a gene product conferring the activity of “glucose-6-phosphate 1-dehydrogenase”, especially from Glycine max or its functional equivalent or its homolog, e.g. the increase of
(a) a gene product of a gene comprising the nucleic acid molecule as shown in the respective line in column 5 of Table I, application no. 10, preferably the coding region thereof, or a homolog or a fragment thereof, and being depicted in the same respective line as said 59582753_SOYBEAN, or a functional equivalent or a homolog thereof as shown in column 8 of Table I, application no. 10, preferably the coding region thereof, particularly a homolog or functional equivalent as shown in column 8 of Table I B, application no. 10, and being depicted in the same respective line as said 59582753_SOYBEAN, and preferably the activity is increased plastidic, or
(b) a polypeptide comprising a polypeptide, a consensus sequence or at least a polypeptide motif as shown in the respective line in column 5 of Table II or in column 8 of Table IV, application no. 10, respectively, and being depicted in the same respective line as said 59582753_SOYBEAN, or a functional equivalent or a homolog thereof as depicted in column 8 of Table II, application no. 10, preferably a homolog or functional equivalent as depicted in column 8 of Table II B, application no. 10, and being depicted in the same respective line as said 59582753_SOYBEAN, and preferably the activity is increased plastidic.

Accordingly, in one embodiment, the molecule which activity is to be increased in the process of the invention is the gene product with an activity as a “glucose-6-phosphate 1-dehydrogenase”, preferably it is the molecule of section (a) or (b) of this paragraph.

In particular, it was observed that in plants, especially in Arabidopsis thaliana, increasing or generating the activity of a gene product plastidic with the activity of a “glucose-6-phosphate 1-dehydrogenase”, preferably being encoded by a gene comprising the nucleic acid sequence SEQ ID NO. 16883, preferably the coding region thereof, conferred the production of or the increase in stearic acid compared with the wild type control.

[0105.1.8.10] to [0107.1.8.10] for the disclosure of these paragraphs see [0105.1.8.8] to [0107.1.8.8] above.

A protein having an activity conferring an increase in the amount or level of the fine chemical stearic acid, upon targeting to the plastids or mitochondria or upon non-targeting, preferably has the structure of the respective polypeptide described herein, in particular of the polypeptides comprising the consensus sequence or at least one polypeptide motifs as shown in the respective line in Table IV, application no. 10, column 8 or of the polypeptide comprising an amino acid sequence as disclosed in the respective line in Table II, application no. 10, columns 5 or 8, or homologs or fragments thereof as described herein, or is encoded by the nucleic acid molecule characterized herein or the nucleic acid molecule according to the invention, for example by the nucleic acid molecule as shown in the respective line in Table I, application no. 10, columns 5 or 8, preferably the coding region thereof, or homologs or fragments thereof and has the herein mentioned activity.

[0109.1.8.10] too [0110.1.8.10] for the disclosure of this paragraph see [0109.1.8.8] to 0110.1.8.8] above.

In one embodiment, the process of the present invention comprises one or more of the following steps
(a) stabilizing a protein conferring the generation or increased expression of a protein encoded by the nucleic acid molecule of the invention or of the polypeptide of the invention, e.g. of a polypeptide having the activity selected from the group consisting of 2-C-methyl-D-erythritol 4-phosphate cytidylyltransferase, 3-deoxy-7-phosphoheptulonate synthase, 3-ketoacyl-CoA thiolase, acetyl-coenzyme A synthetase, acid shock protein, aconitate hydratase, arginine decarboxylase, aspartate-ammonia ligase, At2g45420-protein, ATP-binding component of a transport system, b0283-protein, b0371-protein, b1672-protein, b2032-protein, b2399-protein, b2513-protein, b2849-protein, b3989-protein, b4121-protein, betaine aldehyde dehydrogenase, calcium-dependent protein kinase, cation-transporting ATPase, CCAAT-binding transcription factor, citrate synthase, coproporphyrinogen III oxidase, CTP synthetase, dihydroxyacetone kinase, ethanolamine utilization protein, F-box protein, glucokinase, glucose-6-phosphate 1-dehydrogenase, glutaredoxin, glycerol-3-phosphate dehydrogenase, heat shock transcription factor, isocitrate dehydrogenase, lipoprotein precursor, monothiol glutaredoxin, NADH dehydrogenase subunit N, oxidoreductase, phospho-2-dehydro-3-deoxyheptonate aldolase, protease, protein phosphatase, pyruvate kinase, sll1032-protein, sodium/proton antiporter, transcription factor, tryptophan synthase alpha chain, ycl049c-protein, ydr507c-protein, YGR104C-protein, and yor392w-protein, or of a polypeptide as indicated in the respective line in Table II, application no. 10, columns 5 or 8, or its homologs or fragments, and conferring the production of or an increase in stearic acid, respectively; and/or
(b) stabilizing a mRNA conferring the generation or increased expression of a FCRP, e.g. protein encoded by the nucleic acid molecule of the invention or its homologs or fragments, or of a mRNA encoding the polypeptide of the present invention having the hereinmentioned activity selected from the group consisting of said activities mentioned in (a) and conferring the production of or an increase in stearic acid, respectively; and/or
(c) increasing the specific activity of a protein conferring the increased expression of a FCRP, e.g. a protein encoded by the nucleic acid molecule of the invention or of the polypeptide of the present invention having herein-mentioned stearic acid generating or increasing activity, respectively, e.g. of a polypeptide having the activity of a protein as indicated in the respective line in Table II, application no. 10, columns 5 or 8, or its homologs or fragments, or decreasing the inhibitory regulation of the polypeptide of the invention; and/or
(d) generating or increasing the expression of an endogenous or artificial transcription factor mediating the expression of a protein conferring the generation or increased expression of a FCRP, e.g. a protein encoded by the nucleic acid molecule of the invention or of the polypeptide of the invention having herein-mentioned activity selected from the group consisting of said activities mentioned in (a) and conferring a stearic acid increasing activity, respectively, e.g. of a polypeptide having the activity of a protein as indicated in the respective line in Table II, application no. 10, columns 5 or 8 or its homologs; and/or
(e) stimulating activity of a protein conferring the increased expression of a FCRP, e.g. a protein encoded by the nucleic acid molecule of the present invention or a polypeptide of the present invention having herein-mentioned activity selected from the group consisting of said activities mentioned in (a) and conferring a stearic acid increasing activity, respectively, e.g. of a polypeptide having the activity of a protein as indicated in the respective line in Table II, application no. 10, columns 5 or 8, or its homologs or fragments, by adding one or more exogenous inducing factors to the non-human organism or parts thereof; and/or
(f) expressing a transgenic gene encoding a protein conferring the increased expression of a FCRP, e.g. a polypeptide encoded by the nucleic acid molecule of the present invention or a polypeptide of the present invention, having herein-mentioned activity selected from the group consisting of said activities mentioned in (a) and conferring a stearic acid increasing activity, respectively, e.g. of a polypeptide having the activity of a protein as indicated in the respective line in Table II, application no. 10, columns 5 or 8, or its homologs or fragments, and/or
(g) increasing the copy number of a gene conferring the increased expression of a nucleic acid molecule encoding a FCRP, e.g. a polypeptide encoded by the nucleic acid molecule of the invention or the polypeptide of the invention having herein-mentioned activity selected from the group consisting of said activities mentioned in (a) and conferring a stearic acid increasing activity, respectively, e.g. of a polypeptide having the activity of a protein as indicated in the respective line in Table II, application no. 10, columns 5 or 8, or its homologs or fragments; and/or
(h) increasing the expression of the endogenous gene encoding the FCRP, polypeptide of the invention, e.g. a polypeptide encoded by the nucleic acid molecule of the invention or the polypeptide of the invention having herein-mentioned activity selected from the group consisting of said activities mentioned in (a) and conferring a stearic acid; increasing activity, respectively, e.g. of a polypeptide having the activity of a protein as indicated in the respective line in Table II, application no. 10, columns 5 or 8, or its homologs or fragments, by adding positive expression or removing negative expression elements, e.g. homologous recombination can be used to either introduce positive regulatory elements like for plants the 35S enhancer into the promoter or to remove repressor elements form regulatory regions. Further gene conversion methods can be used to disrupt repressor elements or to enhance to activity of positive elements. Positive elements can be randomly introduced in plants by T-DNA or transposon mutagenesis and lines can be identifled in which the positive elements have be integrated near to a gene of the invention, the expression of which is thereby enhanced; and/or
(i) modulating growth conditions of the non-human organism in such a manner, that the expression or activity of the gene encoding the FCRP, e.g. a protein of the invention or the protein itself is enhanced, for example microorganisms or plants can be grown for example under a higher temperature regime leading to an enhanced expression of heat shock proteins, which can lead to an enhanced stearic acid production, respectively; and/or
(j) selecting of non-human organisms with especially high activity of the proteins of the invention from natural or from mutagenized resources and breeding them into the target organisms, e.g. the elite crops; and/or
(k) directing a protein encoded by the nucleic acid molecule of the invention or of the polypeptide of the present invention having herein-mentioned activity selected from the group consisting of said activities mentioned in (a) and conferring a stearic acid increasing activity, respectively, e.g. of a polypeptide having the activity of a protein as indicated in the respective line in Table II, application no. 10, columns 5 or 8, or its homologs or fragments, if for the corresponding nucleic acid molecule in the respective line in column 6 of Table I the term “plastidic” is indicated, to the plastids by the addition of a plastidial targeting sequence or if for the corresponding nucleic acid molecule in the respective line in column 6 of Table I the term “mitochondrial” is indicated, to the mitochondria by the addition of a mitochondrial targeting sequence; and/or
(l) generating the expression of a protein encoded by the nucleic acid molecule of the invention or of the polypeptide of the present invention having herein-mentioned activity selected from the group consisting of said activities mentioned in (a) and conferring a stearic acid increasing activity, respectively, e.g. of a polypeptide having the activity of a protein as indicated in the respective line in Table II, application no. 10, columns 5 or 8, or its homologs or fragments, if for the corresponding nucleic acid molecule in the respective line in column 6 of Table I the term “plastidic” or “mitochondrial” is indicated, in these organelles by the stable or transient transformation, advantageously stable transformation, of organelles, preferably plastids or mitochondria, with an inventive nucleic acid sequence preferably in form of an expression cassette containing said sequence leading to the expression of the nucleic acids or polypeptides of the invention in the respective organelle; and/or
(m) generating the expression of a protein encoded by the nucleic acid molecule of the invention or of the polypeptide of the present invention having herein-mentioned activity selected from the group consisting of said activities mentioned in (a) and conferring a stearic acid increasing activity, respectively, e.g. of a polypeptide having the activity of a protein as indicated in the respective line in Table II, application no. 10, columns 5 or 8, or its homologs or fragments, if for the corresponding nucleic acid molecule in the respective line in column 6 of Table I the term “plastidic” or “mitochondrial” is indicated, in these organelles by integration of a nucleic acid of the invention into the genome of the respective organelle under control of preferable a promoter selective for the respective organelle.

Preferably, said mRNA is the coding region of the nucleic acid molecule of the present invention and/or the protein conferring the increased expression of a protein encoded by the coding region of the nucleic acid molecule of the present invention alone or linked to a transit nucleic acid sequence or transit peptide encoding nucleic acid sequence or the polypeptide having the herein mentioned activity, e.g. conferring the generation of or increase of stearic acid, respectively, after increasing the expression or activity of the encoded polypeptide, non-targeted or in organelles such as plastids and/or mitochondria, preferably plastids, or having the activity of a polypeptide having an activity as the protein as shown in the respective line in Table II, application no. 10, column 3, or its homologs. Preferably the increase of stearic acid, respectively, takes place non-targeted or in plastids and/or mitochondria, preferably non-targeted or in plastids.

[0113.1.8.10] to [0122.1.8.10] for the disclosure of these paragraphs see [0113.1.8.8] to [0122.1.8.8] above.

The activation of an endogenous polypeptide having above-mentioned activity, e.g. having the activity of a protein as shown in the respective line in Table II, application no. 10, column 3 or of the polypeptide of the invention, e.g. conferring the increase of the fine chemical stearic acid, respectively, by increase of expression or activity in the cytoplasm, and/or in the cytosol, and/or in an organelle, such as plastids or mitochondria, can also be increased by introducing a synthetic transcription factor, which binds close to the coding region of the gene encoding the protein as shown in the respective line in Table II, application no. 10, column 5 or 8, or homologs or fragments thereof, and activates its transcription. A chimeric zinc finger protein can be constructed, which comprises a specific DNA-binding domain and an activation domain as e.g. the VP16 domain of Herpes Simplex virus. The specific binding domain can bind to the regulatory region of the gene encoding the protein as shown in the respective line in Table II, application no. 10, column 5 or 8, or homologs or fragments thereof. The expression of the chimeric transcription factor in a non-human organism, in particular in a plant, leads to a specific expression of the protein as shown in the respective line in Table II, application no. 10, column 5 or 8, or homologs or fragments thereof, see e.g. in W001/52620, Oriz, Proc. Natl. Acad. Sci. USA 99, 13290 (2002), or Guan, Proc. Natl. Acad. Sci. USA 99, 13296 (2002).

[0124.1.8.10] to [0127.1.8.10] for the disclosure of these paragraphs see [0124.1.8.8] to [0127.1.8.8] above.

Owing to the introduction of a gene or a plurality of genes conferring the expression of the nucleic acid molecule of the invention or the polypeptide of the invention, for example the nucleic acid construct mentioned below, or encoding the protein as shown in the respective line in Table II, application no. 10, column 5 or 8, or homologs or fragments thereof, into a non-human organism alone or in combination with other genes, it is possible not only to increase the biosynthetic flux towards the end product, but also to increase, modify or create de novo an advantageous, preferably novel metabolites composition in the non-human organism, e.g. an advantageous composition comprising a higher content of (from a viewpoint of nutritional physiology limited) stearic acid and if desired other fatty acids, and/or other metabolites, in free or bound form.

for the disclosure of this paragraph see [0129.1.8.8] above.

Accordingly, in one embodiment, the process according to the invention relates to a process, which comprises:
(a) providing a non-human organism, preferably a microorganism, a plant cell, a plant tissue, a plant or a part thereof, more preferably a microorganism, a plant tissue, a plant or a part thereof;
(b) increasing the activity of a protein as shown in the respective line in Table II, application no. 10, column 3 or of a polypeptide being encoded by the respective nucleic acid molecule of the present invention and described below, e.g. conferring an increase of the fine chemical stearic acid, respectively, in the non-human organism, preferably in the microorganism, the plant cell, the plant tissue, the plant or a part thereof, more preferably a microorganism, a plant tissue, a plant or a part thereof, especially cytoplasmic or in an organelle, like plastids or mitochondria,
(c) growing the non-human organism, preferably the microorganism, the plant cell, the plant tissue, the plant or a part thereof, under conditions which permit the production of said fine chemical in the non-human organism, preferably the microorganism, the plant cell, the plant tissue, the plant or a part thereof; and
(d) if desired, recovering, optionally isolating, said fine chemical, in free and/or bound form, and, optionally further free and/or bound fatty acids, and/or other metabolites synthesized by the non-human organism, the microorganism, the plant cell, the plant tissue, the plant or a part thereof.

[0131.1.8.10] to [0139.1.8.10] for the disclosure of these paragraphs see [0131.1.8.8] to [0139.1.8.8] above.

In another embodiment, the invention relates to a (isolated) nucleic acid molecule, which comprises a nucleic acid molecule selected from the group consisting of:
(a) a nucleic acid molecule encoding a polypeptide shown in Table II, application no. 10, column 5 or 8, preferably shown in Table II B, application no. 10, column 8, or a homolog or a fragment thereof;
(b) a nucleic acid molecule shown in Table I, application no. 10, column 5 or 8, preferably shown in Table I B, application no. 10, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 10, preferably in column 8 of Table II B, application no. 10;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 10, preferably shown in column 8 of Table I B, application no. 10, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 10;
(i) a nucleic acid molecule encoding a polypeptide having the activity represented by a respective protein as depicted in column 5 of Table II, application no. 10;
(j) a nucleic acid molecule comprising a polynucleotide which is obtained by amplifying a cDNA library or a genomic library using the primers in Table III, application no. 10, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 10.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II application no. 10 and confers a generation or an increase of the respective fine chemical in a non-human organism or a part thereof as compared to a corresponding wild-type non-human organism or part thereof.

In another embodiment, the invention relates to a (isolated) nucleic acid molecule, which comprises a nucleic acid molecule selected from the group consisting of:
(a) a nucleic acid molecule encoding a polypeptide shown in Table II, column 5 or 8, application no. 10, preferably shown in Table II A, application no. 10, in column 5 or in Table II A, application no. 10, column 8 or in Table II B, application no. 10, column 8, or a homolog or a fragment thereof;
(b) a nucleic acid molecule shown in Table I, application no. 10, column 5 or 8, preferably shown in Table I A, application no. 10, in column 5 or in Table I A, application no. 10, column 8 or in Table I B, application no. 10, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 10, preferably shown in Table II A, application no. 10, in column 5 or in Table II A, application no. 10, column 8 or in Table II B, application no. 10, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 10, preferably shown in Table I A, application no. 10, in column 5 or in Table I A, application no. 10, column 8 or in Table I B column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 10;
(i) a nucleic acid molecule encoding a polypeptide having the activity represented by a respective protein as depicted in column 5 of Table II, application no. 10;
(j) a nucleic acid molecule comprising a polynucleotide which is obtained by amplifying a cDNA library or a genomic library using the primers in Table III, application no. 10, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt,500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto;
   whereby, preferably, the nucleic acid molecule according to (a), (b), (c), (d), (e), (f), (g), (h), (i), (j) or (k) distinguishes over the sequence depicted in Table I A and/or I B, application no. 10, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 10, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 10, column 5 or 8, or the coding region thereof, respectively. In a further embodiment the nucleic acid molecule does not encode the polypeptide sequence shown in Table II A and/or II B, application no. 10, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 10, column 5 or 8 and does not encode a protein of the sequence shown in Table II A and/or II B, application no. 10, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 10, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 10, column 5 or 8 but less than 100%, preferably less than 99.999%, 99.99%, 99.9%, 99%, 98%, 97%, 96% or 95% identical to the sequence shown in Table II A and/or II B, application no. 10, column 5 or 8.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 10.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II, application no. 10, and confers a generation or an increase of the respective fine chemical in a non-human organism or a part thereof as compared to a corresponding wild-type non-human organism or part thereof.

[0144.1.8.10] to [0155.1.8.10] for the disclosure of these paragraphs see [0144.1.8.8] to [0155.1.8.8] above.

In a preferred embodiment a nucleic acid construct, for example an expression cassette, comprises upstream, i.e. at the 5′ end of the encoding sequence, a promoter and downstream, i.e. at the 3′ end, a polyadenylation signal and optionally other regulatory elements which are operably linked to the intervening encoding sequence with one of the nucleic acids as depicted in the respective line in Table I, application no. 10, column 5 or 8, preferably the coding region thereof, or a homolog or a fragment thereof. By an operable linkage is meant the sequential arrangement of promoter, encoding sequence, terminator, optionally other regulatory elements and optionally targeting sequences in such a way that each of the regulatory elements/targeting sequence can fulfill its function in the expression of the encoding sequence in due manner. In one embodiment the sequences preferred for operable linkage are targeting sequences for ensuring subcellular localization in organelles, like plastids or mitochondria. However, targeting sequences for ensuring subcellular localization in the endoplasmic reticulum (=ER), in the nucleus, in oil corpuscles or other compartments may also be employed as well as translation promoters such as the 5′ lead sequence in tobacco mosaic virus (Gallie et al., Nucl. Acids Res. 15, 8693 (1987)).

for the disclosure of this paragraph see [0157.1.8.8] above.

In an embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 10, column 5 or 8, preferably shown in Table II A, application no. 10, column 5, or in Table II A, application no. 10, column 8, or in Table II B, application no. 10, column 8, or a homolog or a fragment thereof;
   (b) a nucleic acid molecule shown in Table I, application no. 10, column 5 or 8, preferably shown in Table I A, application no. 10, column 5, or in Table I A, application no. 10, column 8, or in Table I B, application no. 10, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 10, preferably shown in Table II A, application no. 10, column 5, or in Table II A, application no. 10, column 8, or in Table II B, application no. 10, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 10, preferably shown in Table I A, application no. 10, column 5, or in Table I A, application no. 10, column 8, or in Table I B, application no. 10, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 10;
   (i) a nucleic acid molecule encoding a polypeptide having the activity represented by a respective protein as depicted in column 5 of Table II, application no. 10;
   (j) a nucleic acid molecule comprising a polynucleotide which is obtained by amplifying a cDNA library or a genomic library using the primers in Table III, application no. 10, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto. and, optionally
3) nucleic acid molecule encoding a transit peptide, preferably encoding a plastidal transit peptide or a mitochondrial transit peptide;
   which are operable linked.
   (All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 10.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 10 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 10, in the same line as of the respective nucleic acid molecule according to 2) “plastidic” is depicted, or encoding a mitochondrial transit peptide, especially in case in column 6 of Table I, application no. 10, in the same line as of the respective nucleic acid molecule according to 2) “mitochondrial” is depicted.

In another embodiment thereof the expression cassette does not comprise 3) a nucleic acid molecule encoding a transit peptide, especially in case in column 6 of Table I, application no. 10, in the same line as of the respective nucleic acid molecule according to 2) “non-targeted” is depicted.

In a further embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 10, column 5 or 8, preferably shown in Table II A, application no. 10, column 5, or in Table II A, application no. 10, column 8, or in Table II B, application no. 10, column 8, or a homolog or a fragment thereof;
   (b) a nucleic acid molecule shown in Table I, application no. 10, column 5 or 8, preferably shown in Table I A, application no. 10, column 5, or in Table I A, application no. 10, column 8, or in Table I B, application no. 10, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 10, preferably shown in Table II A, application no. 10, column 5, or in Table II A, application no. 10, column 8, or in Table II B, application no. 10, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 10, preferably shown in Table I A, application no. 10, column 5, or in Table I A, application no. 10, column 8,or in Table I B, application no. 10, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 10;
   (i) a nucleic acid molecule encoding a polypeptide having the activity represented by a respective protein as depicted in column 5 of Table II, application no. 10;
   (j) a nucleic acid molecule comprising a polynucleotide which is obtained by amplifying a cDNA library or a genomic library using the primers in Table III, application no. 10, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto; and, optionally
3) nucleic acid molecule encoding a transit peptide, preferably encoding a plastidal transit peptide or a mitochondrial transit peptide;
   which are operable linked and
   whereby, preferably, the nucleic acid molecule according to 2 (a), (b), (c), (d), (e), (f), (g), (h), (i), (j) or (k) distinguishes over the sequence depicted in Table I A and/or I B, application no. 10, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention according to 2) does not consist of the sequence shown in Table I A and/or I B, application no. 10, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 10, column 5 or 8, or the coding region thereof, respectively. In a further embodiment the nucleic acid molecule does not encode the polypeptide sequence shown in Table II A and/or II B, application no. 10, column 5 or 8 but homologs thereof.

Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 10, column 5 or 8 and does not encode a protein of the sequence shown in Table II A and/or II B, application no. 10, column 5 or 8. Accordingly, in one embodiment, the protein encoded by a sequence of a nucleic acid according to 2(a), (b), (c), (d), (e), (f), (g), (h), (i), (j) or (k) does not consist of the sequence shown in Table I A and/or I B, application no. 10, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 10, column 5 or 8 but less than 100%, 99.999%, 99.99%, 99.9%, 99%, 98%, 97%, 96% or 95% identical to the sequence shown in Table II A and/or II B, application no. 10, column 5 or 8.

(All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 10.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 10 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 10, in the same line as of the respective nucleic acid molecule according to 2) “plastidic” is depicted, or encoding a mitochondrial transit peptide, especially in case in column 6 of Table I, application no. 10, in the same line as of the respective nucleic acid molecule according to 2) “mitochondrial” is depicted.

In another embodiment thereof the expression cassette does not comprise 3) a nucleic acid molecule encoding a transit peptide, especially in case in column 6 of Table I, application no. 10, in the same line as of the respective nucleic acid molecule according to 2) “non-targeted” is depicted.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 1061, or a homolog or fragment thereof, or a homolog thereof being depicted in Table I, application no. 10, column 8, in the same line as SEQ ID NO. 1061, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 1061 is depicted, plastidic is mentioned, or a mitochondrial transit peptide in case in Table I, application no. 10, in column 6 in the same line as SEQ ID NO. 1061 is depicted, mitochondrial is mentioned; which is operable linked, too.

In another embodiment thereof the expression cassette does not comprise an additional transit peptide in case in Table I, application no. 10, in column 6 in the same line as SEQ ID NO. 1061 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 1298, or a homolog or fragment thereof, or a homolog thereof being depicted in Table I, application no. 10, column 8, in the same line as SEQ ID NO. 1298, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 1298 is depicted, plastidic is mentioned, or a mitochondrial transit peptide in case in Table I, application no. 10, in column 6 in the same line as SEQ ID NO. 1298 is depicted, mitochondrial is mentioned; which is operable linked, too.

In another embodiment thereof the expression cassette does not comprise an additional transit peptide in case in Table I, application no. 10, in column 6 in the same line as SEQ ID NO. 1298 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 120810, or a homolog or fragment thereof, or a homolog thereof being depicted in Table I, application no. 10, column 8, in the same line as SEQ ID NO. 120810, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 120810 is depicted, plastidic is mentioned, or a mitochondrial transit peptide in case in Table I, application no. 10, in column 6 in the same line as SEQ ID NO. 120810 is depicted, mitochondrial is mentioned; which is operable linked, too.

In another embodiment thereof the expression cassette does not comprise an additional transit peptide in case in Table I, application no. 10, in column 6 in the same line as SEQ ID NO. 120810 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 1623, or a homolog or fragment thereof, or a homolog thereof being depicted in Table I, application no. 10, column 8, in the same line as SEQ ID NO. 1623, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 1623 is depicted, plastidic is mentioned, or a mitochondrial transit peptide in case in Table I, application no. 10, in column 6 in the same line as SEQ ID NO. 1623 is depicted, mitochondrial is mentioned; which is operable linked, too.

In another embodiment thereof the expression cassette does not comprise an additional transit peptide in case in Table I, application no. 10, in column 6 in the same line as SEQ ID NO. 1623 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 120952, or a homolog or fragment thereof, or a homolog thereof being depicted in Table I, application no. 10, column 8, in the same line as SEQ ID NO. 120952, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 120952 is depicted, plastidic is mentioned, or a mitochondrial transit peptide in case in Table I, application no. 10, in column 6 in the same line as SEQ ID NO. 120952 is depicted, mitochondrial is mentioned; which is operable linked, too.

In another embodiment thereof the expression cassette does not comprise an additional transit peptide in case in Table I, application no. 10, in column 6 in the same line as SEQ ID NO. 120952 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 22921, or a homolog or fragment thereof, or a homolog thereof being depicted in Table I, application no. 10, column 8, in the same line as SEQ ID NO. 22921, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 22921 is depicted, plastidic is mentioned, or a mitochondrial transit peptide in case in Table I, application no. 10, in column 6 in the same line as SEQ ID NO. 22921 is depicted, mitochondrial is mentioned; which is operable linked, too.

In another embodiment thereof the expression cassette does not comprise an additional transit peptide in case in Table I, application no. 10, in column 6 in the same line as SEQ ID NO. 22921 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 2935, or a homolog or fragment thereof, or a homolog thereof being depicted in Table I, application no. 10, column 8, in the same line as SEQ ID NO. 2935, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 2935 is depicted, plastidic is mentioned, or a mitochondrial transit peptide in case in Table I, application no. 10, in column 6 in the same line as SEQ ID NO. 2935 is depicted, mitochondrial is mentioned; which is operable linked, too.

In another embodiment thereof the expression cassette does not comprise an additional transit peptide in case in Table I, application no. 10, in column 6 in the same line as SEQ ID NO. 2935 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 23482, or a homolog or fragment thereof, or a homolog thereof being depicted in Table I, application no. 10, column 8, in the same line as SEQ ID NO. 23482, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 23482 is depicted, plastidic is mentioned, or a mitochondrial transit peptide in case in Table I, application no. 10, in column 6 in the same line as SEQ ID NO. 23482 is depicted, mitochondrial is mentioned; which is operable linked, too.

In another embodiment thereof the expression cassette does not comprise an additional transit peptide in case in Table I, application no. 10, in column 6 in the same line as SEQ ID NO. 23482 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 3279, or a homolog or fragment thereof, or a homolog thereof being depicted in Table I, application no. 10, column 8, in the same line as SEQ ID NO. 3279, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 3279 is depicted, plastidic is mentioned, or a mitochondrial transit peptide in case in Table I, application no. 10, in column 6 in the same line as SEQ ID NO. 3279 is depicted, mitochondrial is mentioned; which is operable linked, too.

In another embodiment thereof the expression cassette does not comprise an additional transit peptide in case in Table I, application no. 10, in column 6 in the same line as SEQ ID NO. 3279 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 24232, or a homolog or fragment thereof, or a homolog thereof being depicted in Table I, application no. 10, column 8, in the same line as SEQ ID NO. 24232, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 24232 is depicted, plastidic is mentioned, or a mitochondrial transit peptide in case in Table I, application no. 10, in column 6 in the same line as SEQ ID NO. 24232 is depicted, mitochondrial is mentioned; which is operable linked, too.

In another embodiment thereof the expression cassette does not comprise an additional transit peptide in case in Table I, application no. 10, in column 6 in the same line as SEQ ID NO. 24232 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68849, or a homolog or fragment thereof, or a homolog thereof being depicted in Table I, application no. 10, column 8, in the same line as SEQ ID NO. 68849, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 68849 is depicted, plastidic is mentioned, or a mitochondrial transit peptide in case in Table I, application no. 10, in column 6 in the same line as SEQ ID NO. 68849 is depicted, mitochondrial is mentioned; which is operable linked, too.

In another embodiment thereof the expression cassette does not comprise an additional transit peptide in case in Table I, application no. 10, in column 6 in the same line as SEQ ID NO. 68849 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4102, or a homolog or fragment thereof, or a homolog thereof being depicted in Table I, application no. 10, column 8, in the same line as SEQ ID NO. 4102, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 4102 is depicted, plastidic is mentioned, or a mitochondrial transit peptide in case in Table I, application no. 10, in column 6 in the same line as SEQ ID NO. 4102 is depicted, mitochondrial is mentioned; which is operable linked, too.

In another embodiment thereof the expression cassette does not comprise an additional transit peptide in case in Table I, application no. 10, in column 6 in the same line as SEQ ID NO. 4102 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4348, or a homolog or fragment thereof, or a homolog thereof being depicted in Table I, application no. 10, column 8, in the same line as SEQ ID NO. 4348, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 4348 is depicted, plastidic is mentioned, or a mitochondrial transit peptide in case in Table I, application no. 10, in column 6 in the same line as SEQ ID NO. 4348 is depicted, mitochondrial is mentioned; which is operable linked, too.

In another embodiment thereof the expression cassette does not comprise an additional transit peptide in case in Table I, application no. 10, in column 6 in the same line as SEQ ID NO. 4348 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 25498, or a homolog or fragment thereof, or a homolog thereof being depicted in Table I, application no. 10, column 8, in the same line as SEQ ID NO. 25498, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 25498 is depicted, plastidic is mentioned, or a mitochondrial transit peptide in case in Table I, application no. 10, in column 6 in the same line as SEQ ID NO. 25498 is depicted, mitochondrial is mentioned; which is operable linked, too.

In another embodiment thereof the expression cassette does not comprise an additional transit peptide in case in Table I, application no. 10, in column 6 in the same line as SEQ ID NO. 25498 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 121076, or a homolog or fragment thereof, or a homolog thereof being depicted in Table I, application no. 10, column 8, in the same line as SEQ ID NO. 121076, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 121076 is depicted, plastidic is mentioned, or a mitochondrial transit peptide in case in Table I, application no. 10, in column 6 in the same line as SEQ ID NO. 121076 is depicted, mitochondrial is mentioned; which is operable linked, too.

In another embodiment thereof the expression cassette does not comprise an additional transit peptide in case in Table I, application no. 10, in column 6 in the same line as SEQ ID NO. 121076 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 121111, or a homolog or fragment thereof, or a homolog thereof being depicted in Table I, application no. 10, column 8, in the same line as SEQ ID NO. 121111, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 121111 is depicted, plastidic is mentioned, or a mitochondrial transit peptide in case in Table I, application no. 10, in column 6 in the same line as SEQ ID NO. 121111 is depicted, mitochondrial is mentioned; which is operable linked, too.

In another embodiment thereof the expression cassette does not comprise an additional transit peptide in case in Table I, application no. 10, in column 6 in the same line as SEQ ID NO. 121111 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 121637, or a homolog or fragment thereof, or a homolog thereof being depicted in Table I, application no. 10, column 8, in the same line as SEQ ID NO. 121637, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 121637 is depicted, plastidic is mentioned, or a mitochondrial transit peptide in case in Table I, application no. 10, in column 6 in the same line as SEQ ID NO. 121637 is depicted, mitochondrial is mentioned; which is operable linked, too.

In another embodiment thereof the expression cassette does not comprise an additional transit peptide in case in Table I, application no. 10, in column 6 in the same line as SEQ ID NO. 121637 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7333, or a homolog or fragment thereof, or a homolog thereof being depicted in Table I, application no. 10, column 8, in the same line as SEQ ID NO. 7333, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 7333 is depicted, plastidic is mentioned, or a mitochondrial transit peptide in case in Table I, application no. 10, in column 6 in the same line as SEQ ID NO. 7333 is depicted, mitochondrial is mentioned; which is operable linked, too.

In another embodiment thereof the expression cassette does not comprise an additional transit peptide in case in Table I, application no. 10, in column 6 in the same line as SEQ ID NO. 7333 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 37400, or a homolog or fragment thereof, or a homolog thereof being depicted in Table I, application no. 10, column 8, in the same line as SEQ ID NO. 37400, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 37400 is depicted, plastidic is mentioned, or a mitochondrial transit peptide in case in Table I, application no. 10, in column 6 in the same line as SEQ ID NO. 37400 is depicted, mitochondrial is mentioned; which is operable linked, too.

In another embodiment thereof the expression cassette does not comprise an additional transit peptide in case in Table I, application no. 10, in column 6 in the same line as SEQ ID NO. 37400 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38266, or a homolog or fragment thereof, or a homolog thereof being depicted in Table I, application no. 10, column 8, in the same line as SEQ ID NO. 38266, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 38266 is depicted, plastidic is mentioned, or a mitochondrial transit peptide in case in Table I, application no. 10, in column 6 in the same line as SEQ ID NO. 38266 is depicted, mitochondrial is mentioned; which is operable linked, too.

In another embodiment thereof the expression cassette does not comprise an additional transit peptide in case in Table I, application no. 10, in column 6 in the same line as SEQ ID NO. 38266 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38300, or a homolog or fragment thereof, or a homolog thereof being depicted in Table I, application no. 10, column 8, in the same line as SEQ ID NO. 38300, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 38300 is depicted, plastidic is mentioned, or a mitochondrial transit peptide in case in Table I, application no. 10, in column 6 in the same line as SEQ ID NO. 38300 is depicted, mitochondrial is mentioned; which is operable linked, too.

In another embodiment thereof the expression cassette does not comprise an additional transit peptide in case in Table I, application no. 10, in column 6 in the same line as SEQ ID NO. 38300 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78771, or a homolog or fragment thereof, or a homolog thereof being depicted in Table I, application no. 10, column 8, in the same line as SEQ ID NO. 78771, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 78771 is depicted, plastidic is mentioned, or a mitochondrial transit peptide in case in Table I, application no. 10, in column 6 in the same line as SEQ ID NO. 78771 is depicted, mitochondrial is mentioned; which is operable linked, too.

In another embodiment thereof the expression cassette does not comprise an additional transit peptide in case in Table I, application no. 10, in column 6 in the same line as SEQ ID NO. 78771 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38573, or a homolog or fragment thereof, or a homolog thereof being depicted in Table I, application no. 10, column 8, in the same line as SEQ ID NO. 38573, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 38573 is depicted, plastidic is mentioned, or a mitochondrial transit peptide in case in Table I, application no. 10, in column 6 in the same line as SEQ ID NO. 38573 is depicted, mitochondrial is mentioned; which is operable linked, too.

In another embodiment thereof the expression cassette does not comprise an additional transit peptide in case in Table I, application no. 10, in column 6 in the same line as SEQ ID NO. 38573 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 8920, or a homolog or fragment thereof, or a homolog thereof being depicted in Table I, application no. 10, column 8, in the same line as SEQ ID NO. 8920, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 8920 is depicted, plastidic is mentioned, or a mitochondrial transit peptide in case in Table I, application no. 10, in column 6 in the same line as SEQ ID NO. 8920 is depicted, mitochondrial is mentioned; which is operable linked, too.

In another embodiment thereof the expression cassette does not comprise an additional transit peptide in case in Table I, application no. 10, in column 6 in the same line as SEQ ID NO. 8920 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39237, or a homolog or fragment thereof, or a homolog thereof being depicted in Table I, application no. 10, column 8, in the same line as SEQ ID NO. 39237, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 39237 is depicted, plastidic is mentioned, or a mitochondrial transit peptide in case in Table I, application no. 10, in column 6 in the same line as SEQ ID NO. 39237 is depicted, mitochondrial is mentioned; which is operable linked, too.

In another embodiment thereof the expression cassette does not comprise an additional transit peptide in case in Table I, application no. 10, in column 6 in the same line as SEQ ID NO. 39237 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115628, or a homolog or fragment thereof, or a homolog thereof being depicted in Table I, application no. 10, column 8, in the same line as SEQ ID NO. 115628, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 115628 is depicted, plastidic is mentioned, or a mitochondrial transit peptide in case in Table I, application no. 10, in column 6 in the same line as SEQ ID NO. 115628 is depicted, mitochondrial is mentioned; which is operable linked, too.

In another embodiment thereof the expression cassette does not comprise an additional transit peptide in case in Table I, application no. 10, in column 6 in the same line as SEQ ID NO. 115628 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 79509, or a homolog or fragment thereof, or a homolog thereof being depicted in Table I, application no. 10, column 8, in the same line as SEQ ID NO. 79509, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 79509 is depicted, plastidic is mentioned, or a mitochondrial transit peptide in case in Table I, application no. 10, in column 6 in the same line as SEQ ID NO. 79509 is depicted, mitochondrial is mentioned; which is operable linked, too.

In another embodiment thereof the expression cassette does not comprise an additional transit peptide in case in Table I, application no. 10, in column 6 in the same line as SEQ ID NO. 79509 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 9167, or a homolog or fragment thereof, or a homolog thereof being depicted in Table I, application no. 10, column 8, in the same line as SEQ ID NO. 9167, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 9167 is depicted, plastidic is mentioned, or a mitochondrial transit peptide in case in Table I, application no. 10, in column 6 in the same line as SEQ ID NO. 9167 is depicted, mitochondrial is mentioned; which is operable linked, too.

In another embodiment thereof the expression cassette does not comprise an additional transit peptide in case in Table I, application no. 10, in column 6 in the same line as SEQ ID NO. 9167 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 121657, or a homolog or fragment thereof, or a homolog thereof being depicted in Table I, application no. 10, column 8, in the same line as SEQ ID NO. 121657, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 121657 is depicted, plastidic is mentioned, or a mitochondrial transit peptide in case in Table I, application no. 10, in column 6 in the same line as SEQ ID NO. 121657 is depicted, mitochondrial is mentioned; which is operable linked, too.

In another embodiment thereof the expression cassette does not comprise an additional transit peptide in case in Table I, application no. 10, in column 6 in the same line as SEQ ID NO. 121657 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 81075, or a homolog or fragment thereof, or a homolog thereof being depicted in Table I, application no. 10, column 8, in the same line as SEQ ID NO. 81075, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 81075 is depicted, plastidic is mentioned, or a mitochondrial transit peptide in case in Table I, application no. 10, in column 6 in the same line as SEQ ID NO. 81075 is depicted, mitochondrial is mentioned; which is operable linked, too.

In another embodiment thereof the expression cassette does not comprise an additional transit peptide in case in Table I, application no. 10, in column 6 in the same line as SEQ ID NO. 81075 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 81980, or a homolog or fragment thereof, or a homolog thereof being depicted in Table I, application no. 10, column 8, in the same line as SEQ ID NO. 81980, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 81980 is depicted, plastidic is mentioned, or a mitochondrial transit peptide in case in Table I, application no. 10, in column 6 in the same line as SEQ ID NO. 81980 is depicted, mitochondrial is mentioned; which is operable linked, too.

In another embodiment thereof the expression cassette does not comprise an additional transit peptide in case in Table I, application no. 10, in column 6 in the same line as SEQ ID NO. 81980 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 83657, or a homolog or fragment thereof, or a homolog thereof being depicted in Table I, application no. 10, column 8, in the same line as SEQ ID NO. 83657, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 83657 is depicted, plastidic is mentioned, or a mitochondrial transit peptide in case in Table I, application no. 10, in column 6 in the same line as SEQ ID NO. 83657 is depicted, mitochondrial is mentioned; which is operable linked, too.

In another embodiment thereof the expression cassette does not comprise an additional transit peptide in case in Table I, application no. 10, in column 6 in the same line as SEQ ID NO. 83657 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 100675, or a homolog or fragment thereof, or a homolog thereof being depicted in Table I, application no. 10, column 8, in the same line as SEQ ID NO. 100675, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 100675 is depicted, plastidic is mentioned, or a mitochondrial transit peptide in case in Table I, application no. 10, in column 6 in the same line as SEQ ID NO. 100675 is depicted, mitochondrial is mentioned; which is operable linked, too.

In another embodiment thereof the expression cassette does not comprise an additional transit peptide in case in Table I, application no. 10, in column 6 in the same line as SEQ ID NO. 100675 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 44372, or a homolog or fragment thereof, or a homolog thereof being depicted in Table I, application no. 10, column 8, in the same line as SEQ ID NO. 44372, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 44372 is depicted, plastidic is mentioned, or a mitochondrial transit peptide in case in Table I, application no. 10, in column 6 in the same line as SEQ ID NO. 44372 is depicted, mitochondrial is mentioned; which is operable linked, too.

In another embodiment thereof the expression cassette does not comprise an additional transit peptide in case in Table I, application no. 10, in column 6 in the same line as SEQ ID NO. 44372 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 44609, or a homolog or fragment thereof, or a homolog thereof being depicted in Table I, application no. 10, column 8, in the same line as SEQ ID NO. 44609, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 44609 is depicted, plastidic is mentioned, or a mitochondrial transit peptide in case in Table I, application no. 10, in column 6 in the same line as SEQ ID NO. 44609 is depicted, mitochondrial is mentioned; which is operable linked, too.

In another embodiment thereof the expression cassette does not comprise an additional transit peptide in case in Table I, application no. 10, in column 6 in the same line as SEQ ID NO. 44609 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 10811, or a homolog or fragment thereof, or a homolog thereof being depicted in Table I, application no. 10, column 8, in the same line as SEQ ID NO. 10811, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 10811 is depicted, plastidic is mentioned, or a mitochondrial transit peptide in case in Table I, application no. 10, in column 6 in the same line as SEQ ID NO. 10811 is depicted, mitochondrial is mentioned; which is operable linked, too.

In another embodiment thereof the expression cassette does not comprise an additional transit peptide in case in Table I, application no. 10, in column 6 in the same line as SEQ ID NO. 10811 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 46751, or a homolog or fragment thereof, or a homolog thereof being depicted in Table I, application no. 10, column 8, in the same line as SEQ ID NO. 46751, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 46751 is depicted, plastidic is mentioned, or a mitochondrial transit peptide in case in Table I, application no. 10, in column 6 in the same line as SEQ ID NO. 46751 is depicted, mitochondrial is mentioned; which is operable linked, too.

In another embodiment thereof the expression cassette does not comprise an additional transit peptide in case in Table I, application no. 10, in column 6 in the same line as SEQ ID NO. 46751 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 85510, or a homolog or fragment thereof, or a homolog thereof being depicted in Table I, application no. 10, column 8, in the same line as SEQ ID NO. 85510, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 85510 is depicted, plastidic is mentioned, or a mitochondrial transit peptide in case in Table I, application no. 10, in column 6 in the same line as SEQ ID NO. 85510 is depicted, mitochondrial is mentioned; which is operable linked, too.

In another embodiment thereof the expression cassette does not comprise an additional transit peptide in case in Table I, application no. 10, in column 6 in the same line as SEQ ID NO. 85510 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 85723, or a homolog or fragment thereof, or a homolog thereof being depicted in Table I, application no. 10, column 8, in the same line as SEQ ID NO. 85723, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 85723 is depicted, plastidic is mentioned, or a mitochondrial transit peptide in case in Table I, application no. 10, in column 6 in the same line as SEQ ID NO. 85723 is depicted, mitochondrial is mentioned; which is operable linked, too.

In another embodiment thereof the expression cassette does not comprise an additional transit peptide in case in Table I, application no. 10, in column 6 in the same line as SEQ ID NO. 85723 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 53878, or a homolog or fragment thereof, or a homolog thereof being depicted in Table I, application no. 10, column 8, in the same line as SEQ ID NO. 53878, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 53878 is depicted, plastidic is mentioned, or a mitochondrial transit peptide in case in Table I, application no. 10, in column 6 in the same line as SEQ ID NO. 53878 is depicted, mitochondrial is mentioned; which is operable linked, too.

In another embodiment thereof the expression cassette does not comprise an additional transit peptide in case in Table I, application no. 10, in column 6 in the same line as SEQ ID NO. 53878 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 11471, or a homolog or fragment thereof, or a homolog thereof being depicted in Table I, application no. 10, column 8, in the same line as SEQ ID NO. 11471, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 11471 is depicted, plastidic is mentioned, or a mitochondrial transit peptide in case in Table I, application no. 10, in column 6 in the same line as SEQ ID NO. 11471 is depicted, mitochondrial is mentioned; which is operable linked, too.

In another embodiment thereof the expression cassette does not comprise an additional transit peptide in case in Table I, application no. 10, in column 6 in the same line as SEQ ID NO. 11471 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 55385, or a homolog or fragment thereof, or a homolog thereof being depicted in Table I, application no. 10, column 8, in the same line as SEQ ID NO. 55385, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 55385 is depicted, plastidic is mentioned, or a mitochondrial transit peptide in case in Table I, application no. 10, in column 6 in the same line as SEQ ID NO. 55385 is depicted, mitochondrial is mentioned; which is operable linked, too.

In another embodiment thereof the expression cassette does not comprise an additional transit peptide in case in Table I, application no. 10, in column 6 in the same line as SEQ ID NO. 55385 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 11990, or a homolog or fragment thereof, or a homolog thereof being depicted in Table I, application no. 10, column 8, in the same line as SEQ ID NO. 11990, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 11990 is depicted, plastidic is mentioned, or a mitochondrial transit peptide in case in Table I, application no. 10, in column 6 in the same line as SEQ ID NO. 11990 is depicted, mitochondrial is mentioned; which is operable linked, too.

In another embodiment thereof the expression cassette does not comprise an additional transit peptide in case in Table I, application no. 10, in column 6 in the same line as SEQ ID NO. 11990 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 87655, or a homolog or fragment thereof, or a homolog thereof being depicted in Table I, application no. 10, column 8, in the same line as SEQ ID NO. 87655, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 87655 is depicted, plastidic is mentioned, or a mitochondrial transit peptide in case in Table I, application no. 10, in column 6 in the same line as SEQ ID NO. 87655 is depicted, mitochondrial is mentioned; which is operable linked, too.

In another embodiment thereof the expression cassette does not comprise an additional transit peptide in case in Table I, application no. 10, in column 6 in the same line as SEQ ID NO. 87655 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 58324, or a homolog or fragment thereof, or a homolog thereof being depicted in Table I, application no. 10, column 8, in the same line as SEQ ID NO. 58324, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 58324 is depicted, plastidic is mentioned, or a mitochondrial transit peptide in case in Table I, application no. 10, in column 6 in the same line as SEQ ID NO. 58324 is depicted, mitochondrial is mentioned; which is operable linked, too.

In another embodiment thereof the expression cassette does not comprise an additional transit peptide in case in Table I, application no. 10, in column 6 in the same line as SEQ ID NO. 58324 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 121908, or a homolog or fragment thereof, or a homolog thereof being depicted in Table I, application no. 10, column 8, in the same line as SEQ ID NO. 121908, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 121908 is depicted, plastidic is mentioned, or a mitochondrial transit peptide in case in Table I, application no. 10, in column 6 in the same line as SEQ ID NO. 121908 is depicted, mitochondrial is mentioned; which is operable linked, too.

In another embodiment thereof the expression cassette does not comprise an additional transit peptide in case in Table I, application no. 10, in column 6 in the same line as SEQ ID NO. 121908 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 88879, or a homolog or fragment thereof, or a homolog thereof being depicted in Table I, application no. 10, column 8, in the same line as SEQ ID NO. 88879, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 88879 is depicted, plastidic is mentioned, or a mitochondrial transit peptide in case in Table I, application no. 10, in column 6 in the same line as SEQ ID NO. 88879 is depicted, mitochondrial is mentioned; which is operable linked, too.

In another embodiment thereof the expression cassette does not comprise an additional transit peptide in case in Table I, application no. 10, in column 6 in the same line as SEQ ID NO. 88879 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 113220, or a homolog or fragment thereof, or a homolog thereof being depicted in Table I, application no. 10, column 8, in the same line as SEQ ID NO. 113220, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 113220 is depicted, plastidic is mentioned, or a mitochondrial transit peptide in case in Table I, application no. 10, in column 6 in the same line as SEQ ID NO. 113220 is depicted, mitochondrial is mentioned; which is operable linked, too.

In another embodiment thereof the expression cassette does not comprise an additional transit peptide in case in Table I, application no. 10, in column 6 in the same line as SEQ ID NO. 113220 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 119408, or a homolog or fragment thereof, or a homolog thereof being depicted in Table I, application no. 10, column 8, in the same line as SEQ ID NO. 119408, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 119408 is depicted, plastidic is mentioned, or a mitochondrial transit peptide in case in Table I, application no. 10, in column 6 in the same line as SEQ ID NO. 119408 is depicted, mitochondrial is mentioned; which is operable linked, too.

In another embodiment thereof the expression cassette does not comprise an additional transit peptide in case in Table I, application no. 10, in column 6 in the same line as SEQ ID NO. 119408 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 122228, or a homolog or fragment thereof, or a homolog thereof being depicted in Table I, application no. 10, column 8, in the same line as SEQ ID NO. 122228, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 122228 is depicted, plastidic is mentioned, or a mitochondrial transit peptide in case in Table I, application no. 10, in column 6 in the same line as SEQ ID NO. 122228 is depicted, mitochondrial is mentioned; which is operable linked, too.

In another embodiment thereof the expression cassette does not comprise an additional transit peptide in case in Table I, application no. 10, in column 6 in the same line as SEQ ID NO. 122228 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 90165, or a homolog or fragment thereof, or a homolog thereof being depicted in Table I, application no. 10, column 8, in the same line as SEQ ID NO. 90165, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 90165 is depicted, plastidic is mentioned, or a mitochondrial transit peptide in case in Table I, application no. 10, in column 6 in the same line as SEQ ID NO. 90165 is depicted, mitochondrial is mentioned; which is operable linked, too.

In another embodiment thereof the expression cassette does not comprise an additional transit peptide in case in Table I, application no. 10, in column 6 in the same line as SEQ ID NO. 90165 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 120309, or a homolog or fragment thereof, or a homolog thereof being depicted in Table I, application no. 10, column 8, in the same line as SEQ ID NO. 120309, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 120309 is depicted, plastidic is mentioned, or a mitochondrial transit peptide in case in Table I, application no. 10, in column 6 in the same line as SEQ ID NO. 120309 is depicted, mitochondrial is mentioned; which is operable linked, too.

In another embodiment thereof the expression cassette does not comprise an additional transit peptide in case in Table I, application no. 10, in column 6 in the same line as SEQ ID NO. 120309 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 117482, or a homolog or fragment thereof, or a homolog thereof being depicted in Table I, application no. 10, column 8, in the same line as SEQ ID NO. 117482, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 117482 is depicted, plastidic is mentioned, or a mitochondrial transit peptide in case in Table I, application no. 10, in column 6 in the same line as SEQ ID NO. 117482 is depicted, mitochondrial is mentioned; which is operable linked, too.

In another embodiment thereof the expression cassette does not comprise an additional transit peptide in case in Table I, application no. 10, in column 6 in the same line as SEQ ID NO. 117482 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 66772, or a homolog or fragment thereof, or a homolog thereof being depicted in Table I, application no. 10, column 8, in the same line as SEQ ID NO. 66772, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 66772 is depicted, plastidic is mentioned, or a mitochondrial transit peptide in case in Table I, application no. 10, in column 6 in the same line as SEQ ID NO. 66772 is depicted, mitochondrial is mentioned; which is operable linked, too.

In another embodiment thereof the expression cassette does not comprise an additional transit peptide in case in Table I, application no. 10, in column 6 in the same line as SEQ ID NO. 66772 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 15183, or a homolog or fragment thereof, or a homolog thereof being depicted in Table I, application no. 10, column 8, in the same line as SEQ ID NO. 15183, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 15183 is depicted, plastidic is mentioned, or a mitochondrial transit peptide in case in Table I, application no. 10, in column 6 in the same line as SEQ ID NO. 15183 is depicted, mitochondrial is mentioned; which is operable linked, too.

In another embodiment thereof the expression cassette does not comprise an additional transit peptide in case in Table I, application no. 10, in column 6 in the same line as SEQ ID NO. 15183 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 15532, or a homolog or fragment thereof, or a homolog thereof being depicted in Table I, application no. 10, column 8, in the same line as SEQ ID NO. 15532, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 15532 is depicted, plastidic is mentioned, or a mitochondrial transit peptide in case in Table I, application no. 10, in column 6 in the same line as SEQ ID NO. 15532 is depicted, mitochondrial is mentioned; which is operable linked, too.

In another embodiment thereof the expression cassette does not comprise an additional transit peptide in case in Table I, application no. 10, in column 6 in the same line as SEQ ID NO. 15532 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 16883, or a homolog or fragment thereof, or a homolog thereof being depicted in Table I, application no. 10, column 8, in the same line as SEQ ID NO. 16883, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 10, in column 6 in the same line as SEQ ID NO. 16883 is depicted, plastidic is mentioned, or a mitochondrial transit peptide in case in Table I, application no. 10, in column 6 in the same line as SEQ ID NO. 16883 is depicted, mitochondrial is mentioned; which is operable linked, too.

In another embodiment thereof the expression cassette does not comprise an additional transit peptide in case in Table I, application no. 10, in column 6 in the same line as SEQ ID NO. 16883 is depicted, non-targeted is mentioned.

[0165.1.8.10] to [0170.1.8.10] for the disclosure of these paragraphs see [0165.1.8.8] to [0170.1.8.8] above.

The invention further provides a vector, comprising a nucleic acid molecule comprising 1) a nucleic acid molecule selected from the group consisting of:
(a) a nucleic acid molecule encoding a polypeptide shown in Table II, column 5 or 8, application no. 10, preferably shown in Table II A, application no. 10, in column 5, or in Table II A, application no. 10, column 8, or in Table II B, application no. 10, column 8, or a homolog or a fragment thereof;
(b) a nucleic acid molecule shown in Table I, application no. 10, column 5 or 8, preferably shown in Table I A, application no. 10, in column 5, or in Table I A, application no. 10, column 8, or in Table I B, application no. 10, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 10, preferably shown in Table II A, application no. 10, in column 5, or in Table II A, application no. 10, column 8, or in Table II B, application no. 10, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 10, preferably shown in Table I A, application no. 10, in column 5, or in Table I A, application no. 10, column 8, or in Table I B, application no. 10, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 10;
(i) a nucleic acid molecule encoding a polypeptide having the activity represented by a protein as depicted in column 5 of Table II, application no. 10;
(j) a nucleic acid molecule comprising a polynucleotide which is obtained by amplifying a cDNA library or a genomic library using the primers in Table III, application no. 10, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 10.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 10, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 10.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 10, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 10, in the same line as of the respective nucleic acid molecule according to 1) “plastidic” is depicted, or encoding a mitochondrial transit peptide, especially in case in column 6 of Table I, application no. 10, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 10, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 10, in the same line as of the respective nucleic acid molecule according to 1) “plastidic” is depicted, or encoding a mitochondrial transit peptide, especially in case in column 6 of Table I, application no. 10, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operable linked; a nucleic acid molecule encoding a transit peptide, especially in case in column 6 of Table I, application no. 10, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 10, in the same line as of the respective nucleic acid molecule according to 1) “plastidic” is depicted, or encoding a mitochondrial transit peptide, especially in case in column 6 of Table I, application no. 10, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 10 (in the same line).

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 10, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 10.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 10, in the same line as of the respective nucleic acid molecule according to 1) “plastidic” is depicted, or encoding a mitochondrial transit peptide, especially in case in column 6 of Table I, application no. 10, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 10, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 10, in the same line as of the respective nucleic acid molecule according to 1) “plastidic” is depicted, or encoding a mitochondrial transit peptide, especially in case in column 6 of Table I, application no. 10, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 10 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 10, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 10 (in the same line) and confers a generation or an increase of the respective fine chemical in a non-human organism or a part thereof as compared to a corresponding wild-type non-human organism or part thereof.

In a preferred embodiment of said vector said nucleic acid molecule according to 1 (a), (b), (c), (d), (e), (f), (g), (h), (i), (j) or (k), said promoter and optionally said nucleic acid molecule encoding a transit peptide are encompassed in an expression cassette. Accordingly in an embodiment the invention provides a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

The invention further provides a vector comprising a nucleic acid molecule comprising
1) a nucleic acid molecule selected from the group consisting of:
   (a) a nucleic acid molecule encoding a polypeptide shown in Table II, column 5 or 8, application no. 10, preferably shown in Table II A, application no. 10, in column 5, or in Table II A, application no. 10, column 8, or in Table II B, application no. 10, column 8, or a homolog or a fragment thereof;
   (b) a nucleic acid molecule shown in Table I, application no. 10, column 5 or 8, preferably shown in Table I A, application no. 10, in column 5, or in Table I A, application no. 10, column 8, or in Table I B, application no. 10, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 10, preferably shown in Table II A, application no. 10, in column 5, or in Table II A, application no. 10, column 8, or in Table II B, application no. 10, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 10, preferably shown in Table I A, application no. 10, in column 5, or in Table I A, application no. 10, column 8, or in Table I B, application no. 10, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
   (f) a nucleic acid molecule which hybridizes with a nucleic acid molecule of (a), (b), (c), (d), or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 10;
   (i) a nucleic acid molecule encoding a polypeptide having the activity represented by a protein as depicted in column 5 of Table II, application no. 10;
   (j) a nucleic acid molecule comprising a polynucleotide which is obtained by amplifying a cDNA library or a genomic library using the primers in Table III, application no. 10, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
   or a nucleic acid molecule comprising a sequence which is complementary thereto;
      whereby, preferably, the nucleic acid molecule according to 1 (a), (b), (c), (d), (e), (f), (g), (h), (i), (j) or (k) distinguishes over the sequence depicted in Table I A and/or I B, application no. 10, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 10, column 5 or 8, or the coding region thereof, respectively. In another embodiment, the nucleic acid molecule of the present invention, preferably the coding region thereof, is 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 10, column 5 or 8, or the coding region thereof, respectively. In a further embodiment the nucleic acid molecule does not encode the polypeptide sequence shown in Table II A and/or II B, application no. 10, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 10, column 5 or 8 and does not encode a protein of the sequence shown in Table II A and/or II B, application no. 10, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 10, column 5 and 8, or the coding region thereof. In a further embodiment, the protein of the present invention is at least 30%,40%, 50%, 60%, 70%, 80%, 90%, 95% identical to protein sequence depicted in Table II A and/or II B, application no. 10, column 5 or 8 but less than 100%, preferably less than 99.999%, 99.99% or 99.9%, more preferably less than 99%, 985, 97%, 96% or 95% identical to the sequence shown in Table II A and/or II B, application no. 10, column 5 or 8.
      (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 10.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 10, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 10.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 10, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 10, in the same line as of the respective nucleic acid molecule according to 1) “plastidic” is depicted, or encoding a mitochondrial transit peptide, especially in case in column 6 of Table I, application no. 10, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 10, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 10, in the same line as of the respective nucleic acid molecule according to 1) “plastidic” is depicted, or encoding a mitochondrial transit peptide, especially in case in column 6 of Table I, application no. 10, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 10, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 10, in the same line as of the respective nucleic acid molecule according to 1) “plastidic” is depicted, or encoding a mitochondrial transit peptide, especially in case in column 6 of Table I, application no. 10, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 10.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 10, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 10.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 10, in the same line as of the respective nucleic acid molecule according to 1) “plastidic” is depicted, or encoding a mitochondrial transit peptide, especially in case in column 6 of Table I, application no. 10, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 10, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 10, in the same line as of the respective nucleic acid molecule according to 1) “plastidic” is depicted, or encoding a mitochondrial transit peptide, especially in case in column 6 of Table I, application no. 10, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 10 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 10, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 10 and confers a generation or an increase of the respective fine chemical in a non-human organism or a part thereof as compared to a corresponding wild-type non-human organism or part thereof.

[0181.1.8.10] to [0209.1.8.10] for the disclosure of these paragraphs see [0181.1.8.8] to [0209.1.8.8] above.

The genes of the invention, coding for an activity selected from the group consisting of 2-C-methyl-D-erythritol 4-phosphate cytidylyltransferase, 3-deoxy-7-phosphoheptulonate synthase, 3-ketoacyl-CoA thiolase, acetyl-coenzyme A synthetase, acid shock protein, aconitate hydratase, arginine decarboxylase, aspartate-ammonia ligase, At2g45420-protein, ATP-binding component of a transport system, b0283-protein, b0371-protein, b1672-protein, b2032-protein, b2399-protein, b2513-protein, b2849-protein, b3989-protein, b4121-protein, betaine aldehyde dehydrogenase, calcium-dependent protein kinase, cation-transporting ATPase, CCAAT-binding transcription factor, citrate synthase, coproporphyrinogen III oxidase, CTP synthetase, dihydroxyacetone kinase, ethanolamine utilization protein, F-box protein, glucokinase, glucose-6-phosphate 1-dehydrogenase, glutaredoxin, glycerol-3-phosphate dehydrogenase, heat shock transcription factor, isocitrate dehydrogenase, lipoprotein precursor, monothiol glutaredoxin, NADH dehydrogenase subunit N, oxidoreductase, phospho-2-dehydro-3-deoxyheptonate aldolase, protease, protein phosphatase, pyruvate kinase, sll1032-protein, sodium/proton antiporter, transcription factor, tryptophan synthase alpha chain, ycl049c-protein, ydr507c-protein, YGR104C-protein, and yor392w-protein are also called “FCRP genes”.

[0211.1.8.10] to [0225.1.8.10] for the disclosure of these paragraphs see [0211.1.8.8] to [0225.1.8.8] above.

In addition to the sequence mentioned in Table I, application no. 10, column 5 or 8, preferably the coding region thereof, or its homologs or fragments, it may be advantageous additionally to express and/or mutate further genes in the non-human organisms. Especially advantageously, additionally at least one further gene of the biosynthetic pathway of the fine chemical is expressed in the non-human organisms such as plants or microorganisms. It is also possible that the regulation of the natural genes has been modified advantageously so that the gene and/or its gene product is no longer subject to the regulatory mechanisms which exist in the non-human organisms. This leads to an increased synthesis of the respective fatty acid since, for example, feedback regulations no longer exist to the same extent or not at all. In addition it might be advantageously to combine the nucleic acids sequences of the invention containing the sequences shown in the respective line in Table I, application no. 10, column 5 or 8, preferably the coding region thereof, or homologs or fragments thereof, with genes which generally support or enhance the growth or yield of the target non-human organisms, for example genes which lead to faster growth rate of non human organism like microorganisms or plants or genes which produce stress-, pathogen-, or herbicide-resistant plants.

[0227.1.8.10] to [0239.1.8.10] for the disclosure of these paragraphs see [0227.1.8.8] to [0239.1.8.8] above.

In an embodiment of the present invention the nucleic acid construct of the present invention comprises a nucleic acid molecule as depicted in the respective line in Table I, application no. 10, column 5 or 8, preferably the coding region thereof, a fragment or a homolog thereof, and a plant promoter, such as the Big 35S promoter, the DC3 promoter, the PC Ubi promoter, the LegB4-promoter, the Super promoter, the USP promoter or the like. In another embodiment the nucleic acid construct of the present invention comprises a nucleic acid molecule as depicted in the respective line in Table I, application no. 10, column 5 or 8, preferably the coding region thereof, a fragment or a homolog thereof, and a plant promoter such as, for example, the USP, the LegB4-, the DC3 promoter or the ubiquitin promoter from parsley or other herein mentioned promoters. In a further embodiment the nucleic acid construct of the present invention comprises a nucleic acid molecule as depicted in the respective line in Table I, application no. 10, column 5 or 8, preferably the coding region thereof, a fragment or a homolog thereof, and a plant promoter, such as the Big 35S promoter, the PC Ubi promoter, the Super promoter, the USP promoter or the like. In addition different terminators may advantageously be used in these nucleic acid constructs; examples for transcriptional termination are polyadenylation signals.

[0241.1.8.10] to [0245.1.8.10] for the disclosure of these paragraphs see [0241.1.8.8] to [0245.1.8.8] above.

Other preferred sequences for use in operable linkage in gene expression constructs are targeting sequences, which are required for targeting the gene product into specific cell compartments (for a review, see Kermode, Crit. Rev. Plant Sci. 15 (4), 285 (1996) and references cited therein), for example into the vacuole, the nucleus, all types of plastids, such as amyloplasts, chloroplasts, chromoplasts, the extracellular space, the mitochondria, the endoplasmic reticulum, elaioplasts, peroxisomes, glycosomes, and other compartments of cells or extracellular; preferred are sequences, which are involved in targeting to plastids as mentioned above. Sequences, which must be mentioned in this context are, in particular, the signalpeptideor transit-peptide-encoding sequences which are known per se. For example, plastidtransit-peptide-encoding sequences enable the targeting of the expression product into the plastids of a plant cell. Targeting sequences are also known for eukaryotic and to a lower extent for prokaryotic organisms and can advantageously be operable linked with the nucleic acid molecule of the present invention as shown in Table I, application no. 10, columns 5 and 8, preferably the coding region thereof, or homologs or fragments thereof, and described herein to achieve an expression in one of said compartments or extracellular.

[0247.1.8.10] to [0266.1.8.10] for the disclosure of these paragraphs see [0247.1.8.8] to [0266.1.8.8] above.

An embodiment of the invention relates to a vector where the nucleic acid molecule according to the invention is linked operably to regulatory sequences which permit the expression in a prokaryotic or eukaryotic host. A further embodiment of the invention relates to a vector in which a nucleic acid sequence encoding one of the polypeptides shown in the respective line in Table II, application no. 10, columns 5 or 8, or homologs or fragments thereof, is functionally linked to a targeting sequence such as a plastidial or mitochondrial targeting sequence. A further embodiment of the invention relates to a vector in which a nucleic acid sequence encoding one of the polypeptides shown in the respective line in Table II, application no. 10, columns 5 or 8, or homologs or fragments thereof, is functionally linked to (a) regulatory sequence which permit the expression in an organelle, such as plastids or mitochondria, preferably plastids.

A further embodiment of the invention relates to a vector which comprises a nucleic acid sequence encoding one of the polypeptides shown in the respective line in Table II, application no. 10, columns 5 or 8, or homologs or fragments thereof, but no additional targeting sequence.

[0268.1.8.10] to [0273.1.8.10] for the disclosure of these paragraphs see [0268.1.8.8] to [0273.1.8.9] above.

A further object of the invention relates to the use of a nucleic acid construct, e.g. an expression cassette, comprising one or more nucleic acid molecules encoding one or more polypeptides as depicted in the respective line(s) in Table II, application no. 10, columns 5 or 8, or homologs or fragments thereof, or comprising one or more nucleic acid molecules as depicted in the respective line(s) in Table I, application no. 10, columns 5 or 8, preferably the coding region thereof, or homologs or fragments thereof, or comprising a nucleic acid molecule hybridizing therewith for the transformation of a microorganism or a plant cell, plant tissues or a part of a plant.

In doing so, depending on the choice of promoter, the nucleic acid molecules shown in the respective line in Table I, application no. 10, column 5 or 8, preferably the coding region thereof, or homologs or fragments thereof, or the nucleic acid molecules encoding a polypeptide as depicted in the respective line in Table II, application no. 10, column 5 or 8, or homologs or fragments thereof, can be expressed in the whole plant or specifically e.g. in the leaves, in the seeds, the nodules, in roots, in the stem or other parts of the plant. Those transgenic plants overproducing sequences, e.g. as depicted in the respective line in Table I, application no. 10, column 5 or 8, preferably the coding region thereof, or homologs or fragments thereof, the plant cell, plant tissues, plants, the reproductive material thereof, or parts thereof are a further object of the present invention.

Within the framework of the present invention, the production of the stearic acid is due to the generation or over-expression of one or more polypeptides as depicted in the respective line(s) in Table II, application no. 10, column 5 or 8, or homologs or fragments thereof, or encoded by the corresponding nucleic acid molecules as depicted in the respective line(s) in Table I, application no. 10, column 5 or 8, preferably the coding region thereof, or homologs or fragments thereof, in the non-human organism according to the invention, advantageously in the transgenic microorganism or plant according to the invention, at least for the duration of at least one plant generation.

A constitutive expression of the polypeptide as depicted in the respective line in Table II, application no. 10, column 5 or 8, or homologs or fragments thereof, or encoded by the respective nucleic acid molecule as depicted in the respective line in Table I, application no. 10, column 5 or 8, preferably the coding region thereof, or homologs or fragments thereof, may be advantageous. On the other hand, an inducible expression may also appear desirable. Expression of the polypeptide sequences of the invention can be either directed to the cytosol or to the organelles, such as plastids or mitochondria, preferably the plastids of the host cells, preferably the plant cells, or non-targeted.

for the disclosure of this paragraph see [0278.1.8.8] above.

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 10, column 5 or 8, preferably shown in Table II A, application no. 10, column 5, or in Table II A, application no. 10, column 8, or in Table II B, application no. 10, column 8, or a homolog or a fragment thereof;
   (b) a nucleic acid molecule shown in Table I, application no. 10, column 5 or 8, preferably shown in Table I A, application no. 10, column 5, or in Table I A, application no. 10, column 8, or in Table I B, application no. 10, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 10, preferably shown in Table II A, application no. 10, column 5, or in Table II A, application no. 10, column 8, or in Table II B, application no. 10, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 10, preferably shown in Table I A, application no. 10, column 5, or in Table I A, application no. 10, column 8, or in Table I B, application no. 10, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 10;
   (i) a nucleic acid molecule encoding a polypeptide having the activity represented by a respective protein as depicted in column 5 of Table II, application no. 10;
   (j) a nucleic acid molecule comprising a polynucleotide which is obtained by amplifying a cDNA library or a genomic library using the primers in Table III, application no. 10, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising an expression cassette comprising
[0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 10, column 5 or 8, preferably shown in Table II A, application no. 10, column 5, or in Table II A, application no. 10, column 8, or in Table II B, application no. 10, column 8, or a homolog or a fragment thereof;
   (b) a nucleic acid molecule shown in Table I, application no. 10, column 5 or 8, preferably shown in Table I A, application no. 10, column 5, or in Table I A, application no. 10, column 8, or in Table I B, application no. 10, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 10, preferably shown in Table II A, application no. 10, column 5, or in Table II A, application no. 10, column 8, or in Table II B, application no. 10, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 10, preferably shown in Table I A, application no. 10, column 5, or in Table I A, application no. 10, column 8, or in Table I B, application no. 10, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 10;
   (i) a nucleic acid molecule encoding a polypeptide having the activity represented by a respective protein as depicted in column 5 of Table II, application no. 10;
   (j) a nucleic acid molecule comprising a polynucleotide which is obtained by amplifying a cDNA library or a genomic library using the primers in Table III, application no. 10, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising or being transformed by a vector comprising
[1) a nucleic acid molecule selected from the group consisting of
   (a) a nucleic acid molecule encoding a polypeptide shown in Table II, application no. 10, column 5 or 8, preferably shown in Table II A, application no. 10, column 5, or in Table II A, application no. 10, column 8, or in Table II B, application no. 10, column 8, or a homolog or a fragment thereof;
   (b) a nucleic acid molecule shown in Table I, application no. 10, column 5 or 8, preferably shown in Table I A, application no. 10, column 5, or in Table I A, application no. 10, column 8, or in Table I B, application no. 10, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 10, preferably shown in Table II A, application no. 10, column 5, or in Table II A, application no. 10, column 8, or in Table II B, application no. 10, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 10, preferably shown in Table I A, application no. 10, column 5, or in Table I A, application no. 10, column 8, or in Table I B, application no. 10, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 10;
   (i) a nucleic acid molecule encoding a polypeptide having the activity represented by a respective protein as depicted in column 5 of Table II, application no. 10;
   (j) a nucleic acid molecule comprising a polynucleotide which is obtained by amplifying a cDNA library or a genomic library using the primers in Table III, application no. 10, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 10.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 10 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 10, in the same line as of the respective nucleic acid molecule according to 1) “plastidic” is depicted, or encoding a mitochondrial transit peptide, especially in case in column 6 of Table I, application no. 10, in the same line as of the respective nucleic acid molecule according to 1) “mitochondrial” is depicted.

In another embodiment of said transgenic non-human organism the expression cassette does not comprise 2) a nucleic acid molecule encoding a transit peptide, especially in case in column 6 of Table I, application no. 10, in the same line as of the respective nucleic acid molecule according to 1) “non-targeted” is depicted.

[0282.1.10.10] In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 10.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 10, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 10, in the same line as of the respective nucleic acid molecule according to 1) “plastidic” is depicted, or encoding a mitochondrial transit peptide, especially in case in column 6 of Table I, application no. 10, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 10, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 10, in the same line as of the respective nucleic acid molecule according to 1) “plastidic” is depicted, or encoding a mitochondrial transit peptide, especially in case in column 6 of Table I, application no. 10, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 10, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 10, in the same line as of the respective nucleic acid molecule according to 1) “plastidic” is depicted, or encoding a mitochondrial transit peptide, especially in case in column 6 of Table I, application no. 10, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 10.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 10, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 10.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 10, in the same line as of the respective nucleic acid molecule according to 1) “plastidic” is depicted, or encoding a mitochondrial transit peptide, especially in case in column 6 of Table I, application no. 10, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 10, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 10, in the same line as of the respective nucleic acid molecule according to 1) “plastidic” is depicted, or encoding a mitochondrial transit peptide, especially in case in column 6 of Table I, application no. 10, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 10 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 10, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 10 (in the same line) and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 10, column 5 or 8, preferably shown in Table II A, application no. 10, column 5, or in Table II A, application no. 10, column 8, or in Table II B, application no. 10, column 8, or a homolog or a fragment thereof;
   (b) a nucleic acid molecule shown in Table I, application no. 10, column 5 or 8, preferably shown in Table I A, application no. 10, column 5, or in Table I A, application no. 10, column 8, or in Table I B, application no. 10, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 10, preferably shown in Table II A, application no. 10, column 5, or in Table II A, application no. 10, column 8, or in Table II B, application no. 10, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 10, preferably shown in Table I A, application no. 10, column 5, or in Table I A, application no. 10, column 8, or in Table I B, application no. 10, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 10;
   (i) a nucleic acid molecule encoding a polypeptide having the activity represented by a respective protein as depicted in column 5 of Table II, application no. 10;
   (j) a nucleic acid molecule comprising a polynucleotide which is obtained by amplifying a cDNA library or a genomic library using the primers in Table III, application no. 10, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising an expression cassette comprising
[0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 10, column 5 or 8, preferably shown in Table II A, application no. 10, column 5, or in Table II A, application no. 10, column 8, or in Table II B, application no. 10, column 8, or a homolog or a fragment thereof;
   (b) a nucleic acid molecule shown in Table I, application no. 10, column 5 or 8, preferably shown in Table I A, application no. 10, column 5, or in Table I A, application no. 10, column 8, or in Table I B, application no. 10, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 10, preferably shown in Table II A, application no. 10, column 5, or in Table II A, application no. 10, column 8, or in Table II B, application no. 10, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 10, preferably shown in Table I A, application no. 10, column 5, or in Table I A, application no. 10, column 8, or in Table I B, application no. 10, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 10;
   (i) a nucleic acid molecule encoding a polypeptide having the activity represented by a respective protein as depicted in column 5 of Table II, application no. 10;
   (j) a nucleic acid molecule comprising a polynucleotide which is obtained by amplifying a cDNA library or a genomic library using the primers in Table III, application no. 10, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      and, if desired
2) nucleic acid molecule encoding a transit peptide, preferably encoding a plastidal transit peptide or a mitochondrial transit peptide;
   which are operable linked;]
   or comprising or being transformed by a vector comprising
[1) a nucleic acid molecule selected from the group consisting of
   (a) a nucleic acid molecule encoding a polypeptide shown in Table II, application no. 10, column 5 or 8, preferably shown in Table II A, application no. 10, column 5, or in Table II A, application no. 10, column 8, or in Table II B, application no. 10, column 8, or a homolog or a fragment thereof;
   (b) a nucleic acid molecule shown in Table I, application no. 10, column 5 or 8, preferably shown in Table I A, application no. 10, column 5, or in Table I A, application no. 10, column 8, or in Table I B, application no. 10, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 10, preferably shown in Table II A, application no. 10, column 5, or in Table II A, application no. 10, column 8, or in Table II B, application no. 10, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 10, preferably shown in Table I A, application no. 10, column 5, or in Table I A, application no. 10, column 8, or in Table I B, application no. 10, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 10;
   (i) a nucleic acid molecule encoding a polypeptide having the activity represented by a respective protein as depicted in column 5 of Table II, application no. 10;
   (j) a nucleic acid molecule comprising a polynucleotide which is obtained by amplifying a cDNA library or a genomic library using the primers in Table III, application no. 10, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      whereby, preferably, the nucleic acid molecule according to 1 (a), (b), (c), (d), (e), (f), (g), (h), (i), (j) or (k) distinguishes over the sequence depicted in Table I A and/or I B, application no. 10, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 10, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 10, column 5 or 8, or the coding region thereof, respectively. In a further embodiment the nucleic acid molecule does not encode the polypeptide sequence shown in Table II A and/or II B, application no. 10, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 10, column 5 or 8 and does not encode a protein of the sequence shown in Table II A and/or II B, application no. 10, column 5 or 8. Accordingly, in one embodiment, the protein encoded by a sequence of a nucleic acid according to 1 (a), (b), (c), (d), (e), (f), (g), (h), (i), (j) or (k) does not consist of the sequence shown in Table I A and/or I B, application no. 10, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 10, column 5 or 8 but less than 100%, preferably less than 99.999%, 99.99%, 99.9%, 99%, 98%, 97%, 96% or 95% identical to the sequence shown in Table II A and/or II B, application no. 10, column 5 or 8;

as well as respective transgenic cells, tissue, parts of such non-human organism, e.g. plant cells, plant tissue, part of plants, like leaves, roots, stems, blossoms, seeds, fruits, pollen and the like.

(All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 10.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 10 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 10, in the same line as of the respective nucleic acid molecule according to 1) “plastidic” is depicted, or encoding a mitochondrial transit peptide, especially in case in column 6 of Table I, application no. 10, in the same line as of the respective nucleic acid molecule according to 1) “mitochondrial” is depicted.

In another embodiment thereof the expression cassette does not comprise 2) a nucleic acid molecule encoding a transit peptide, especially in case in column 6 of Table I, application no. 10, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 10.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the same line in column 5 of Table II, application no. 10, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 10, in the same line as of the respective nucleic acid molecule according to 1) “plastidic” is depicted, or encoding a mitochondrial transit peptide, especially in case in column 6 of Table I, application no. 10, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 10, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 10, in the same line as of the respective nucleic acid molecule according to 1) “plastidic” is depicted, or encoding a mitochondrial transit peptide, especially in case in column 6 of Table I, application no. 10, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 10, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 10, in the same line as of the respective nucleic acid molecule according to 1) “plastidic” is depicted, or encoding a mitochondrial transit peptide, especially in case in column 6 of Table I, application no. 10, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 10.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 10, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 10.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 10, in the same line as of the respective nucleic acid molecule according to 1) “plastidic” is depicted, or encoding a mitochondrial transit peptide, especially in case in column 6 of Table I, application no. 10, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 10, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 10, in the same line as of the respective nucleic acid molecule according to 1) “plastidic” is depicted, or encoding a mitochondrial transit peptide, especially in case in column 6 of Table I, application no. 10, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 10 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 10, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 10 and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

[0291.1.8.10] to [0299.1.8.10] for the disclosure of these paragraphs see [0291.1.8.8] to [0299.1.8.8] above.

The term “biological active portion” or “biological activity” means a polypeptide as depicted in the respective line in Table II, application no. 10, column 3 or a portion of said polypeptide which still has at least 10% or 20%, preferably 30%, 40%, 50% or 60%, especially preferably 70%, 75%, 80%, 90% or 95% of the enzymatic or biological activity of the natural or starting enzyme or protein.

[0301.1.8.10] to [0304.1.8.10] for the disclosure of these paragraphs see [0301.1.8.8] to [0304.1.8.8] above.

Synthetic oligonucleotide primers for the amplification, e.g. as shown in the respective line in Table III, application no. 10, column 8, by means of polymerase chain reaction can be generated on the basis of a sequence shown herein, for example the sequence shown in the respective line in Table I, application no. 10, columns 5 or 8, or the sequences derived from Table II, application no. 10, columns 5 or 8, respectively.

Moreover, it is possible to identify a conserved region of a protein from various organism by carrying out protein sequence alignments with the polypeptide encoded by the nucleic acid molecules of the present invention, in particular with the sequences encoded by the nucleic acid molecule shown in the respective line in column 5 or 8 of Table I, application no. 10, from which conserved regions, and in turn, degenerate primers can be derived. Conserved regions are those, which show a very little variation in the amino acid in one particular position of several homologs from different origin. The consensus sequence and polypeptide motifs shown in the respective line in column 8 of Table IV, application no. 10, are derived from such alignments of homologous proteins.

In an embodiment of the present invention, in the method of the present invention the activity of a polypeptide comprising or consisting of a consensus sequence or at least one polypeptide motif shown in the respective line in Table IV, application no. 10, column 8, is increased. In another embodiment thereof, the method of the present invention relates to a polypeptide comprising or consisting of a consensus sequence shown in the respective line in Table IV, application no. 10, column 8 or comprising at least one polypeptide motif shown in the respective line in Table IV, application no. 10, column 8, whereby not more than 20, 15, 10, 9, 8, 7, 6, 5 4, 3, 2 or 1, or 0 of the amino acid positions indicated can be replaced by any amino acid. In another embodiment thereof, the method of the present invention relates to a polypeptide comprising or consisting of a consensus sequence shown in the respective line in Table IV, application no. 10, column 8 or comprising at least one polypeptide motif shown in the respective line in Table IV, application no. 10, column 8, whereby not more than 15%, 10%, 5%, 4%, 3%, 2% or 1%, or 0% of the amino acid positions defined as distinct specific amino acids are/is replaced by another amino acid. In another embodiment thereof, the method of the present invention relates to a polypeptide comprising or consisting of a consensus sequence shown in the respective line in Table IV, application no. 10, column 8 or comprising at least one polypeptide motif shown in the respective line in Table IV, application no. 10, column 8, whereby not more than 20, 15, 10, 9, 8, 7, 6, 5, 4, 3, 2 or 1, or 0 amino acids are inserted into a consensus sequence or polypeptide motif.

In an embodiment of the present invention, in the method of the present invention the activity of a polypeptide comprising or consisting of a consensus sequence or at least one polypeptide motif shown in the respective line in Table IV, application no. 10, column 8, is increased, whereby said polypeptide distinguishes over the sequence depicted in Table II, application no. 10, columns 5 or 8 by one or more amino acids.

In one embodiment, said polypeptide of the invention distinguishes over the sequence shown in the respective line in Table II, application no. 10, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II B, application no. 10, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in Table II, application no. 10, columns 5 or 8.

[0309.1.8.10] to [0321.1.8.10] for the disclosure of these paragraphs see [0309.1.8.8] to [0321.1.8.8] above.

Polypeptides having above-mentioned activity, i.e. conferring the production or the increased production of the fine chemical stearic acid as compared to a corresponding, e.g.

non-transformed, wild type non-human organism, like a microorganism or a plant cell, plant or part thereof, can be encoded by other DNA sequences which hybridize to the sequences shown in the respective line in Table I, application no. 10, columns 5 and 8, preferably the coding region thereof, at least under relaxed hybridization conditions and which encode the expression of polypeptides conferring the production or the increased production of the respective fine chemical stearic acid as compared to a corresponding, e.g. non-transformed, wild type non-human organism, like a microorganism or a plant cell, plant or part thereof.

[0323.1.8.10] to [0329.1.8.10] for the disclosure of these paragraphs see [0323.1.8.8] to [0329.1.8.8] above.

Further, the nucleic acid molecule of the invention comprises a nucleic acid molecule, which is a complement of one of the nucleotide sequences of above-mentioned nucleic acid molecules or a portion thereof. A nucleic acid molecule or its sequence which is complementary to one of the nucleotide molecules or sequences shown in the respective line in Table I, application no. 10, columns 5 or 8, preferably the coding region thereof, homologs or fragments thereof, is one which is sufficiently complementary to one of the nucleotide molecules or sequences shown in the respective line in Table I, application no. 10, columns 5 or 8, preferably the coding region thereof, homologs or fragments thereof, such that it can hybridize to one of the nucleotide sequences shown in Table I, application no. 10, columns 5 or 8, preferably the coding region thereof, homologs or fragments thereof, thereby forming a stable duplex. Preferably, the hybridization is performed under stringent hybridization conditions. However, a complement of one of the herein disclosed sequences is preferably a sequence complement thereto according to the base pairing of nucleic acid molecules well known to the skilled person. For example, the bases A and G undergo base pairing with the bases T and U or C, resp. and visa versa. Modifications of the bases can influence the base-pairing partner.

The nucleic acid molecule of the invention comprises a nucleotide sequence which is at least about 30%, 35%, 40%, 45%, 50%, 55%, 60%, 65%, 70%, 75%, 80%, 90%, 95%, 96%, 97%, 98%, 99%, 99.5% or more homologous to a nucleotide sequence shown in Table I, application no. 10, columns 5 or 8, preferably the coding region thereof, homologs or fragments thereof, and preferably has above-mentioned activity, in particular conferring the production or the increased production of the fine chemical stearic acid, respectively, after increasing the activity or an activity of a gene as shown in the respective line in Table I or of a gene product, e.g. as shown in the respective line in Table II, application no. 10, column 5 or 8, by for example in one embodiment expression either in the cytosol or in an organelle such as a plastid or mitochondria or both, preferably in a plastid, or in another embodiment non-targeted or targeted.

The nucleic acid molecule of the invention comprises a nucleotide sequence or molecule which hybridizes, preferably hybridizes under stringent conditions as defined herein, to one of the nucleic acid molecule shown in the respective line in Table I, preferably Table IB, application no. 10, columns 5 or 8, preferably the coding region thereof, fragments or homologs thereof, and encodes a protein having above-mentioned activity, e.g. conferring the production or the increased production of the respective fine chemical stearic acid as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted, and optionally, the activity thereof is selected from the group consisting of 2-C-methyl-D-erythritol 4-phosphate cytidylyltransferase, 3-deoxy7-phosphoheptulonate synthase, 3-ketoacyl-CoA thiolase, acetyl-coenzyme A synthetase, acid shock protein, aconitate hydratase, arginine decarboxylase, aspartate-ammonia ligase, At2g45420-protein, ATP-binding component of a transport system, b0283-protein, b0371-protein, b1672-protein, b2032-protein, b2399-protein, b2513-protein, b2849-protein, b3989-protein, b4121-protein, betaine aldehyde dehydrogenase, calcium-dependent protein kinase, cation-transporting ATPase, CCAAT-binding transcription factor, citrate synthase, coproporphyrinogen III oxidase, CTP synthetase, dihydroxyacetone kinase, ethanolamine utilization protein, F-box protein, glucokinase, glucose-6-phosphate 1-dehydrogenase, glutaredoxin, glycerol-3-phosphate dehydrogenase, heat shock transcription factor, isocitrate dehydrogenase, lipoprotein precursor, monothiol glutaredoxin, NADH dehydrogenase subunit N, oxidoreductase, phospho-2-dehydro-3-deoxyheptonate aldolase, protease, protein phosphatase, pyruvate kinase, sll1032-protein, sodium/proton antiporter, transcription factor, tryptophan synthase alpha chain, ycl049c-protein, ydr507c-protein, YGR104C-protein, and yor392w-protein, respectively.

Moreover, the nucleic acid molecule of the invention can comprise only a portion of the coding region of one of the nucleic acid molecules depicted in Table I, application no. 10, columns 5 or 8, for example a fragment which can be used as a probe or primer or a fragment encoding a biologically active portion of the polypeptide of the present invention or of a polypeptide used in the process of the present invention, i.e. having above-mentioned activity, e.g. conferring the production or the increased production of the respective fine chemical stearic acid as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted. The nucleotide sequences determined from the cloning of the present protein-according-to-the-invention-encoding gene allows for the generation of probes and primers designed for use in identifying and/or cloning its homologues from other cell types and organisms. The probe/primer typically comprises a substantially purified oligonucleotide. The oligonucleotide typically comprises a region of nucleotide sequence that hybridizes under stringent conditions to at least about 12, 15 preferably about 20 or 25, more preferably about 40, 50 or 75 consecutive nucleotides of a sense strand of one of the sequences set forth, e.g., in the respective line in Table I, application no. 10, column 5 or 8, preferably the coding region thereof, or a homolog or a fragment thereof, an anti-sense sequence of one of the sequences, e.g., set forth in the respective line in Table I, application no. 10, columns 5 or 8, preferably the coding region thereof, or a homolog or a fragment thereof or naturally occurring mutants thereof. Primers based on a nucleic acid molecule of the invention can be used in PCR reactions to clone homologs of the polypeptide of the invention or of the polypeptide used in the process of the invention, e.g. as the primers described in the examples of the present invention, e.g. as shown in the examples. A PCR with the primers shown in the respective line in Table III, column 8 will result in a fragment of the gene product as shown in Table II, application no. 10, column 5.

Primer sets are interchangeable. The person skilled in the art knows how to design and combine said primers to result in the desired product, e.g. in a full length clone or a partial sequence. Probes based on the sequences of the nucleic acid molecule of the invention or used in the process of the present invention can be used to detect transcripts or genomic sequences encoding the same or homologous proteins. The probe can further comprise a label group attached thereto, e.g. the label group can be a radioisotope, a fluorescent compound, an enzyme, or an enzyme co-factor. Such probes can be used as a part of a genomic marker test kit for identifying cells which express a polypeptide of the invention or used in the process of the present invention, such as by measuring a level of an encoding nucleic acid molecule in a sample of cells, e.g. detecting mRNA levels or determining, whether a genomic gene comprising the sequence of the polynucleotide of the invention or used in the processes of the present invention has been mutated or deleted.

As the expression of a respective nucleic acids of the invention is related to the synthesis of the respective fine chemical stearic acid its function as a probe extends to the detection of microorganisms, plant tissues, plants, plant variets, plant ecotypes or plant genera with varying capability or potential for synthesis of the respective fine chemical stearic acid. Therefore in one embodiment the present invention relates to a method for analyzing the capability or potential of a plant tissue, a plant, a plant variety or plant ecotype to produce the fine chemical stearic acid by using the nucleic acid of the invention or parts thereof as a probe to detect the amount of the nucleic acid of the invention in the non-human organism or a part thereof in comparison to another non-human organism.

The nucleic acid molecule of the invention encodes a polypeptide or portion thereof which includes an amino acid sequence which is sufficiently homologous to the amino acid sequence shown in respective line in Table II, application no. 10, columns 5 or 8 such that the protein or portion thereof maintains the ability to confer the production or the increased production of the respective fine chemical stearic acid as compared to a corresponding, e.g. non-transformed, wild type non-human organism, like a microorganism or a plant cell, plant or part thereof, in particular increasing the activity as mentioned above or as described in the examples in microorganisms or plants.

As used herein, the language “sufficiently homologous” refers to proteins or portions thereof which have amino acid sequences which include a minimum number of identical or equivalent amino acids (e.g. an amino acid which has a similar side chain as an amino acid in one of the sequences of the polypeptide of the present invention) to an amino acid sequence shown in the respective line in Table II, application no. 10, columns 5 or 8 such that the protein or portion thereof is able to confer the production or the increased production of the respective fine chemical stearic acid as compared to a corresponding, e.g. non-transformed, wild type non-human organism, like a microorganism or a plant cell, plant or part thereof. For example having the activity of a protein as shown in the respective line in Table II, application no. 10, column 3 and as described herein.

In an embodiment of the present invention, the nucleic acid molecule of the present invention comprises a nucleic acid molecule that encodes a portion of the protein of the present invention. The protein is at least about 30%, 35%, 40%, 45% 50%, 55%, 60%, 65%, 70%, 75%, 80%, 85%, 90%, 91%, 92%, 93%, 94%, 95%, 96%, 97%, 98%, 99%, 99.5% or more homologous to an entire amino acid sequence shown in the respective line of Table II, application no. 10, column 5 or 8 and has the above-mentioned activity, e.g. conferring the production or the increased production of the respective fine chemical stearic acid as compared to a corresponding, e.g. non-transformed, wild type non-human organism, like a microorganism or a plant cell, plant or part thereof by, for example in a embodiment expression either in the cytosol or in an organelle such as a plastid or mitochondria or both, preferably in a plastid, or in another embodiment by targeted or non-targeted expression.

[0338.1.8.10] to [0339.1.8.10] for the disclosure of these paragraphs see [0338.1.8.8] to [0339.1.8.8] above.

The invention further relates to nucleic acid molecules or methods using said nucleic acid molecules that differ from one of the nucleotide sequences shown in the respective line in Table I, application no. 10, columns 5 or 8, preferably the coding region thereof, or fragments or homologs thereof, due to degeneracy of the genetic code but encode a polypeptide of the present invention, in particular a polypeptide having the above-mentioned activity, e.g. as those polypeptides depicted in the respective line in Table II, application no. 10, columns 5 or 8, fragments or homologs thereof. Advantageously, the nucleic acid molecule of the invention comprises, or in an other embodiment has, a nucleotide sequence encoding a protein comprising, or in an other embodiment having, an amino acid sequence shown in the respective line in Table II, application no. 10, columns 5 or 8, fragments or homologs thereof. In a further embodiment, the nucleic acid molecule of the invention encodes a full length protein which is substantially homologous to an amino acid sequence shown in the respective line in Table II, application no. 10, columns 5 or 8, or homologs thereof. However, in an embodiment, the nucleic acid molecule of the present invention does not consist of the sequence shown in the respective line in Table I, preferably Table IA, application no. 10, columns 5 or 8, preferably the coding region thereof, but the functional homologs thereof.

[0341.1.8.10] to [0343.1.8.10] for the disclosure of these paragraphs see [0341.1.8.8] to [0343.1.8.8] above.

Accordingly, in another embodiment, a nucleic acid molecule of the invention is at least 15, 20, 25 or 30 nucleotides in length. Preferably, it hybridizes under stringent conditions to a nucleic acid molecule comprising a nucleotide sequence of the nucleic acid molecule of the present invention or used in the process of the present invention, e.g. comprising the sequence shown in the respective line in Table I, application no. 10, columns 5 or 8, preferably the coding region thereof. In another embodiment the nucleic acid molecule is preferably at least 20, 30, 50, 100, 250 or more nucleotides in length.

for the disclosure of this paragraph see [0345.1.8.8] above.

Preferably, a nucleic acid molecule of the invention that hybridizes under stringent conditions to a sequence shown in the respective line in Table I, application no. 10, columns 5 or 8, preferably the coding region thereof, corresponds to a naturally-occurring nucleic acid molecule of the invention. As used herein, a “naturally-occurring” nucleic acid molecule refers to a RNA or DNA molecule having a nucleotide sequence that occurs in nature (e.g. encodes a natural protein). Preferably, the nucleic acid molecule encodes a natural protein having above-mentioned activity, e.g. conferring the production or the increased production of the respective fine chemical stearic acid as compared to a corresponding, e.g. non-transformed, wild type non-human organism, like a microorganism or a plant cell, plant or part thereof after increasing the expression or activity thereof or the activity of a protein of the invention or used in the process of the invention, in an embodiment for example expression either in the cytosol or in an organelle such as a plastid or mitochondria, preferably in plastids, or, in another embodiment by targeted or non-targeted expression.

for the disclosure of this paragraph see [0347.1.8.8] above.

For example, nucleotide substitutions leading to amino acid substitutions at “non-essential” amino acid residues can be made in a sequence of the nucleic acid molecule of the invention or used in the process of the invention, e.g. shown in the respective line in Table I, application no. 10, columns 5 or 8, preferably the coding region thereof, or fragments or homologs thereof.

[0349.1.8.10] to [0350.1.8.10] for the disclosure of these paragraphs see [0349.1.8.8] to [0350.1.8.8] above.

Accordingly, the invention relates to nucleic acid molecules encoding a polypeptide having above-mentioned activity, in a non-human organisms or parts thereof by for example expression either in the cytosol or in an organelle such as a plastid or mitochondria or both, preferably in plastids, that contain changes in amino acid residues that are not essential for said activity. Such polypeptides differ in amino acid sequence from a sequence depicted in the respective line in Table II, especially Table IIA, application no. 10, columns 5 or 8, but retain said activity described herein. The nucleic acid molecule can comprise a nucleotide sequence encoding a polypeptide, wherein the polypeptide comprises an amino acid sequence at least about 50% identical to an amino acid sequence shown in the respective line in Table II, application no. 10, columns 5 or 8, and is capable of conferring the production or the increased production of the respective fine chemical stearic acid as compared to a corresponding, e.g. non-transformed, wild type non-human organism, like a microorganism or a plant cell, plant or part thereof, after increasing its activity for example in an embodiment by expression either in the cytosol or in an organelle such as a plastid or mitochondria or both, preferably in plastids, or, in another embodiment by targeted or non-targeted expression. Preferably, the protein encoded by the nucleic acid molecule is at least about 60%, 70%, 80%, 90%, 95%, 96%, 97%, 98%, 99% or 99.5% identical to the sequence shown in the respective line in Table II, application no. 10, columns 5 or 8.

[0352.1.8.10] to [0357.1.8.10] for the disclosure of these paragraphs see [0352.1.8.8] to [0357.1.8.8] above.

Functional equivalents of the nucleic acid molecules according to the present invention and/or used in the process according to the present invention represent an embodiment of homologs. Functional equivalents derived from the nucleic acid molecules as shown in the respective line in Table I, application no. 10, columns 5 or 8, preferably the coding region thereof, according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the nucleic acid molecules as shown in the respective line in Table I, application no. 10, columns 5 or 8, preferably the coding region thereof, according to the invention and encode polypeptides having essentially the same properties as the polypeptide as shown in the respective line in Table II, application no. 10, columns 5 or 8.

Functional equivalents derived from one of the polypeptides as shown in the respective line in Table II, application no. 10, columns 5 or 8 according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the polypeptides as shown in the respective line in Table II, application no. 10, columns 5 or 8 according to the invention and having essentially the same properties as the polypeptide as shown in the respective line in Table II, application no. 10, columns 5 or 8.

for the disclosure of this paragraph see [0359.1.8.8] above.

A nucleic acid molecule encoding a homolog to a polypeptide depicted in the respective line in Table II, application no. 10, columns 5 or 8 can be created by introducing one or more nucleotide substitutions, additions or deletions into the nucleic acid molecule of the present invention, in particular of Table I, application no. 10, columns 5 or 8, in the respective line, such that one or more amino acid substitutions, additions or deletions are introduced into the encoded protein. Mutations can be introduced into the nucleic acid molecules as depicted in the respective line in Table I, application no. 10, columns 5 or 8, preferably the coding region thereof, by standard techniques, such as site-directed mutagenesis, PCR-mediated mutagenesis or other methods known to the person skilled in the art.

[0361.1.8.10] to [0363.1.8.10] for the disclosure of these paragraphs see [0361.1.8.8] to [0363.1.8.8] above.

Homologues of the nucleic acid sequences used, with the sequences shown in the respective line in Table I, application no. 10, columns 5 or 8, preferably the coding region thereof, comprise also allelic variants with at least approximately 30%, 35%, 40%, 45%, 50%, 55%, 60%, 65%, 70%, 75%, 80%, 85%, 90%, 91%, 92%, 93%, 94% 95%. 96%, 97%, 98%, 99%, 99.5% or more % homology with one of the nucleotide sequences shown or the abovementioned derived nucleic acid sequences or their homologues or derivatives or parts of these. Allelic variants encompass in particular functional variants which can be obtained by deletion, insertion or substitution of nucleotides from the sequences shown in the respective line, preferably in Table I, columns 5 or 8, preferably the coding region thereof, or from the derived nucleic acid sequences, the intention being, however, that the enzymatic activity or the biological activity of the resulting proteins synthesized is advantageously retained or increased.

In one embodiment of the present invention, the nucleic acid molecule of the invention or used in the process of the invention comprises the sequences shown in the respective line in any of the Table I, application no. 10, columns 5 or 8, preferably the coding region thereof, fragments or homologs thereof. It is preferred that the nucleic acid molecule comprises as little as possible other nucleotides not shown in the respective line in any one of Table I, application no. 10, columns 5 or 8, preferably the coding region thereof, fragments or homologs thereof. In one embodiment, the nucleic acid molecule comprises less than 500, 400, 300, 200, 100, 90, 80, 70, 60, 50 or 40 further nucleotides. In a further embodiment, the nucleic acid molecule comprises less than 30, 20 or 10 further nucleotides. In one embodiment, the nucleic acid molecule used in the process of the invention is identical to the sequences shown in the respective line in Table I, application no. 10, columns 5 or 8, preferably the coding region thereof.

Also preferred is that the nucleic acid molecule used in the process of the invention encodes a polypeptide comprising the sequence shown in the respective line in Table II, application no. 10, columns 5 or 8, fragments or homologs thereof. In one embodiment, the nucleic acid molecule encodes less than 150, 130, 100, 80, 60, 50, 40 or 30 further amino acids. In a further embodiment, the encoded polypeptide comprises less than 20, 15, 10, 9, 8, 7, 6 or 5 further amino acids. In one embodiment used in the inventive process, the encoded polypeptide is identical to the sequences shown in the respective line in Table II, application no. 10, columns 5 or 8.

In one embodiment, the nucleic acid molecule of the invention or used in the process encodes a polypeptide comprising the sequence shown in the respective line in Table II, application no. 10, columns 5 or 8, or fragments or homologs thereof, and comprises less than 100 further nucleotides. In a further embodiment, said nucleic acid molecule comprises less than 30 further nucleotides. In one embodiment, the nucleic acid molecule used in the process is identical to a coding sequence of the sequences shown in the respective line in Table I, application no. 10, columns 5 or 8.

Polypeptides (=proteins), which still have the essential biological or enzymatic activity of the polypeptide of the present invention conferring the production or the increased production of the respective fine chemical stearic acid as compared to a corresponding, e.g. non-transformed, wild type non-human organism, like a microorganism or a plant cell, plant or part thereof, i.e. whose activity is essentially not reduced, are polypeptides with at least 10%, 20%, 30%, 40%, 50%, 60%, 70%, 80%, 90%, 95% or more of the wild type biological activity or enzymatic activity, advantageously, the activity is essentially not reduced in comparison with the activity of a polypeptide shown in the respective line in Table II, application no. 10, columns 5 or 8 expressed under identical conditions.

Homologues of nucleic acid molecules shown in the respective line in Table I, application no. 10, columns 5 or 8, preferably the coding region thereof, or fragments thereof, or of the derived sequences shown in the respective line in Table II, application no. 10, columns 5 or 8, or fragments thereof, also mean truncated sequences, cDNA, single-stranded DNA or RNA of the coding and noncoding DNA sequence. Homologues of said sequences are also understood as meaning derivatives, which comprise noncoding regions such as, for example, UTRs, introns, terminators, enhancers or promoter variants. The promoters upstream of the nucleotide sequences stated can be modified by one or more nucleotide substitution(s), insertion(s) and/or deletion(s) without, however, interfering with the functionality or activity either of the promoters, the open reading frame (=ORF) or with the 3′-regulatory region such as terminators or other 3′-regulatory regions, which are far away from the ORF. It is furthermore possible that the activity of the promoters is increased by modification of their sequence, or that they are replaced completely by more active promoters, even promoters from heterologous organisms. Appropriate promoters are known to the person skilled in the art and are mentioned herein below.

[0370.1.8.10] to [0379.1.8.10] for the disclosure of these paragraphs see [0370.1.8.8] to [0379.1.8.8] above.

Moreover, a native polypeptide conferring the increase of the respective fine chemical stearic acid in a non-human organism or a part thereof can be isolated from cells (e.g., endothelial cells), for example using the antibody of the present invention as described below, in particular, an antibody against proteins having 2-C-methyl-D-erythritol 4-phosphate cytidylyltransferase, 3-deoxy-7-phosphoheptulonate synthase, 3-ketoacyl-CoA thiolase, acetylcoenzyme A synthetase, acid shock protein, aconitate hydratase, arginine decarboxylase, aspartate-ammonia ligase, At2g45420-protein, ATP-binding component of a transport system, b0283-protein, b0371-protein, b1672-protein, b2032-protein, b2399-protein, b2513-protein, b2849-protein, b3989-protein, b4121-protein, betaine aldehyde dehydrogenase, calcium-dependent protein kinase, cation-transporting ATPase, CCAAT-binding transcription factor, citrate synthase, coproporphyrinogen III oxidase, CTP synthetase, dihydroxyacetone kinase, ethanolamine utilization protein, F-box protein, glucokinase, glucose-6-phosphate 1-dehydrogenase, glutaredoxin, glycerol-3-phosphate dehydrogenase, heat shock transcription factor, isocitrate dehydrogenase, lipoprotein precursor, monothiol glutaredoxin, NADH dehydrogenase subunit N, oxidoreductase, phospho-2-dehydro-3-deoxyheptonate aldolase, protease, protein phosphatase, pyruvate kinase, sll1032-protein, sodium/proton antiporter, transcription factor, tryptophan synthase alpha chain, ycl049c-protein, ydr507c-protein, YGR104C-protein, or yor392w-protein activity, respectively, or an antibody against polypeptides as shown in the respective line in Table II, application no. 10, columns 5 or 8, or fragments or homologs thereof which can be produced by standard techniques utilizing the polypeptid of the present invention or fragment thereof, i.e., the polypeptide of this invention (FCRP). Preferred are monoclonal antibodies.

for the disclosure of this paragraph see [0381.1.8.8] above.

In one embodiment, the present invention relates to a polypeptide as depicted in the respective line in Table II, especially in Table IIA or especially in Table IIB, application no. 10, columns 5 or 8, or fragment or homolog thereof or as coded by the nucleic acid molecule shown in the respective line in Table I, especially Table IA or especially Table IB, application no. 10, columns 5 or 8, preferably the coding region thereof, or fragment or homolog thereof.

In an embodiment, the present invention relates to a polypeptide comprising or consisting of a consensus sequence shown in the respective line in Table IV, application no. 10, column 8 or at least one polypeptide motif shown in the respective line in Table IV, application no. 10, column 8.

In an embodiment thereof, the present invention relates to a polypeptide comprising or consisting of a consensus sequence shown in the respective line in Table IV, application no. 10, column 8 or comprising at least one polypeptide motif shown in the respective line in Table IV, application no. 10, column 8, whereby not more than 20, 15, 10, 9, 8, 7, 6, 5, 4, 3, 2, or 1, or 0 of the amino acids can be replaced by any amino acid.

In another embodiment thereof, the present invention relates to a polypeptide comprising or consisting of a consensus sequence shown in the respective line in Table IV, application no. 10, column 8 or comprising at least one polypeptide motif shown in the respective line in Table IV, application no. 10, column 8, whereby not more than 15%, 10%, 5%, 4%, 3%, 2% or1% or 0% of the amino acids defined as distinct specific amino acids are/is replaced by another amino acid.

In another embodiment thereof, the present invention relates to a polypeptide comprising or consisting of a consensus sequence shown in the respective line in Table IV, application no. 10, column 8 or comprising at least one polypeptide motif shown in the respective line in Table IV, application no. 10, column 8, whereby not more than 20, 15, 10, 9, 8, 7, 6, 5, 4, 3, 2, or 1, or 0 amino acids are inserted into the consensus sequence or polypeptide motif.

In an embodiment of the present invention, the polypeptide of the present invention comprises or consists of a consensus sequence or at least one polypeptide motif shown in the respective line in Table IV, application no. 10, column 8, whereby said polypeptide distinguishes over the sequence depicted in the respective line in Table II, application no. 10, columns 5 or 8 by one or more amino acids. In one embodiment, said polypeptide of the invention distinguishes over the sequence shown in the respective line in Table II, application no. 10, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 10, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in the respective line in Table II, application no. 10, columns 5 and 8.

In an embodiment, the invention relates to a polypeptide conferring an increase in the respective fine chemical stearic acid in a non-human organism, especially a microorganism or a plant, or a part thereof, being encoded by the nucleic acid molecule of the invention or used in the process of the invention and having a sequence which distinguishes over the sequence as shown in the respective line in Table II, application no. 10, columns 5 or 8 by one or more amino acids (but not exceeding 5%, preferably 4%, 3%, 2%, 1%, 0.5%, 0.2% or 0.1%). In an embodiment, said polypeptide of the invention does not comprise or consist of the sequence shown in the respective line in Table II, application no. 10, columns 5 or 8. In an embodiment, said polypeptide of the present invention is less than 100%, 99.999%, 99.99%, 99.9% or 99% identical. In one embodiment, said polypeptide which differs at least in one or more amino acids (but not exceeding 5%, preferably 4%, 3%, 2%, 1%, 0.5%, 0.2% or 0.1%) from the polypeptide shown in the respective line in Table II, application no. 10, columns 5 and 8 does not comprise a protein of the sequence shown in the respective line in Table II A and/or II B, application no. 10, columns 5 or 8.

In an embodiment, the present invention relates to a polypeptide having the activity of the protein as shown in the respective line in Table II, application no. 10, column 3, which distinguishes over the sequence depicted in the respective line in Table II, application no. 10, columns 5 or 8 by one or more amino acids, preferably by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 10, columns 5 and 8 by not more than 80%, 70%, 60%, 50%, 40%, 30%, 20%, 10%, 5%, 4%, 3%, 2%, 1% or 0.5%.

In another embodiment the polypeptide of the invention takes the form of a preprotein consisting of a plastidial or mitochondrial transit peptide joint to a polypeptide having the activity of the protein as shown in Table II, column 3, from which the transit peptide is preferably cleaved off upon transport of the preprotein into the organelle for example into the plastid or mitochondrion.

In another embodiment the polypeptide of the invention takes not the form of a preprotein.

[0390.1.8.10] to [0391.1.8.10] for the disclosure of these paragraphs see [0390.1.8.8] to [0391.1.8.8] above.

A polypeptide of the invention can participate in the process of the present invention. The polypeptide or a portion thereof comprises preferably an amino acid sequence, which is sufficiently homologous to an amino acid sequence shown in the respective line in Table II A and/or II B, application no. 10, columns 5 or 8.

Further, the polypeptide can have an amino acid sequence which is encoded by a nucleotide sequence which hybridizes, preferably hybridizes under stringent conditions as described above, to a nucleotide sequence of the nucleic acid molecule of the present invention. Accordingly, the polypeptide has an amino acid sequence which is encoded by a nucleotide sequence that is at least about 35%, 40%, 45%, 50%, 55%, 60%, 65%, 70%, 75%, 80%, 85%, 90%, 91%, 92%, 93%, 94%, 95%, 96%, 97%, 98%, 99%, 99.5% or more homologous to one of the amino acid sequences as shown in the respective line in Table II A and/or II B, application no. 10, columns 5 or 8, or fragments thereof. The preferred polypeptide of the present invention preferably possesses at least one of the activities according to the invention and described herein. A preferred polypeptide of the present invention includes an amino acid sequence encoded by a nucleotide sequence which hybridizes, preferably hybridizes under stringent conditions, to a nucleotide sequence shown in the respective line in Table I A and/or I B, application no. 10, columns 5 or 8, preferably the coding region thereof, or fragments thereto or which is homologous thereto, as defined above.

The polypeptide of the present invention can vary from the sequences shown in the respective line in Table II A and/or II B, application no. 10, columns 5 or 8 in amino acid sequence due to natural variation or mutagenesis, as described in detail herein. Accordingly, the polypeptide comprise an amino acid sequence which is at least about 35%, 40%, 45%, 50%, 55%, 60%, 65%, 70%, 75%, 80%, 85% or 90, 91%, 92%, 93%, 94%, 95%, 96%, 97%, 98%, 99%, 99.5% or more homologous to an entire amino acid sequence shown in the respective line in Table II A and/or II B, application no. 10, columns 5 or 8.

Biologically active portions of a polypeptide of the present invention include peptides comprising amino acid sequences derived from the amino acid sequence of the polypeptide of the present invention or used in the process of the present invention, e.g., the amino acid sequence shown in the respective line in Table II, application no. 10, columns 5 or 8 or the amino acid sequence of a protein homologous thereto, which include fewer amino acids than a full length polypeptide of the present invention or used in the process of the present invention or the full length protein which is homologous to a polypeptide of the present invention or used in the process of the present invention depicted herein, and exhibit at least one activity of a polypeptide of the present invention or used in the process of the present invention.

for the disclosure of this paragraph see [0396.1.8.8] above.

Manipulation of the nucleic acid molecule of the invention may result in the production of a protein having differences from the protein as shown in the respective line in Table II, application no. 10, column 5 or 8. Differences shall mean at least one amino acid different from the sequences as shown in the respective line in Table II, application no. 10, column 5 or 8, preferably at least 2, 3, 4, 5, 6, 7, 8, 9 or 10 amino acids (especially but not exceeding 0.1%, preferably 0.2%, 0.5%, 1%, 2%, 3% 4% or 5%), more preferably at least 15, 20, 25, 30, 35, 40, 45 or 50 amino acids (especially but not exceeding 0.5%, preferably 1%, 2%, 3% 4%, 5%, 10% or 25%) different from the sequences as shown in the respective line in Table II, application no. 10, column 5 or 8. These proteins may be improved in efficiency or activity, or may be more stable and therefore present in greater numbers in the cell as compared to the wild-type cell.

Any mutagenesis strategies for the polypeptide of the present invention or the polypeptide used in the process of the present invention to result in increasing said activity are not meant to be limiting; variations on these strategies will be readily apparent to one skilled in the art. Using such strategies, and incorporating the mechanisms disclosed herein, the nucleic acid molecule and polypeptide of the invention may be utilized to generate algae, ciliates, fungi, other microorganisms like C. glutamicum or plants or parts thereof, expressing proteins as shown in the respective line in Table II, application no. 10, column 5 or 8, or mutated proteins thereof. The nucleic acid molecules and polypeptide molecules of the invention are expressed such that the yield, production, and/or efficiency of production of a desired compound is improved.

[0399.1.8.10] to [0401.1.8.10] for the disclosure of these paragraphs see [0399.1.8.8] to [0401.1.8.8] above.

In one embodiment, a protein (=polypeptide) as shown in the respective line in Table II, application no. 10, column 5 or 8 refers to a polypeptide having an amino acid sequence corresponding to the polypeptide of the invention, especially to a polypeptide as shown in Table II, application no. 10, columns 5 or 8 or homologs thereof, or used in the process of the invention, whereas a “non-inventive protein or polypeptide” or “other polypeptide” refers to a polypeptide having an amino acid sequence corresponding to a protein which is not substantially homologous to a polypeptide of the invention, preferably which is not substantially homologous to a polypeptide or protein as shown in the respective line in Table II, application no. 10, column 5 or 8 or homologs thereof, e.g., a protein which does not confer the activity described herein and which is derived from the same or a different organism.

[0403.1.8.10] to [0409.1.8.10] for the disclosure of these paragraphs see [0403.1.8.8] to [0409.1.8.8] above.

As the nucleic acid molecules of the invention and/or the expression thereof is related to the synthesis of the respective fine chemical stearic acid its function as a probe extends to the detection of microorganisms, plant tissues, plants, plant variets, plant ecotypes or plant genera with varying, advantageously increased, capability or potential for synthesis of the respective fine chemical stearic acid. Therefore in one embodiment the present invention relates to a method for analyzing the capability or potential of a plant tissue, a plant, a plant variety or ecotype to produce the respective fine chemical stearic acid by using the respective antibody of the invention as a probe to detect the amount of the polypeptide encoded by said nucleic acid molecule of the invention in a non-human organism in comparison to another organism.

Therefore in one embodiment the invention relates to a method to probe a non-human organism for its capability for the production of the respective fine chemical comprising the steps of
a) analyzing the sequence and/or the expression of the respective nucleic acid molecule of the invention in least two non-human organisms which differ in the capability to produce the respective fine chemical;
b) associate the different sequence and/or expression level of the nucleic acid molecules of the invention with their different capability to produce the respective fine chemical;
c) analyze the sequence and/or the expression of the nucleic acid molecule of the invention in at least a third non-human organism;
d) predict the capability of the at least third organism to produce the respective fine chemical based by comparing the sequence and/or expression of the nucleic acid of the invention to the sequences and/or expression levels of the nucleic acid molecule of the invention of the at least two organisms analyzed in step a.

[0411.1.8.10] to [0430.1.8.10] for the disclosure of these paragraphs see [0411.1.8.8] to [0430.1.8.8] above.

Accordingly the present invention relates to any cell transgenic for any nucleic acid characterized as part of the invention, e.g. conferring the increase of the respective fine chemical stearic acid in a cell or a non-human organism or a part thereof, e.g. the nucleic acid molecule of the invention, the nucleic acid construct of the invention, the vector of the invention, the expression cassette according to the invention, or a nucleic acid molecule encoding the polypeptide of the invention, e.g. encoding a polypeptide having an activity as the protein as shown in the respective line in Table II, application no. 10, column 3. Due to the abovementioned activity the respective fine chemical stearic acid content in a cell or a non-human organism is increased. For example, due to modulation or manipulation, the cellular activity is increased, in a preferred embodiment in organelles such as plastids or mitochondria, e.g. due to an increased expression or specific activity or specific targeting of the subject matters of the invention in a cell or a non-human organism or a part thereof especially in organelles such as plastids or mitochondria, or in another embodiment in the cytosol. Transgenic for a polypeptide having a protein or a protein activity means herein that due to modulation or manipulation of the genome, the activity of protein as shown in the respective line in Table II, application no. 10, column 3 or a protein as shown in the respective line in Table II, application no. 10, column alike activity is increased in the cell or non-human organism or part thereof, especially in organelles such as plastids or mitochondria, or especially in the cytosol. Examples are described above in context with the process of the invention.

for the disclosure of this paragraph see [0432.1.8.8] above.

A naturally occurring expression cassette—for example the naturally occurring combination of the promoter of the gene encoding a protein as shown in the respective line in Table II, application no. 10, column 5 or 8, preferably the coding region thereof, or fragments or homologs thereof with the corresponding encoding gene—becomes a transgenic expression cassette when it is modified by non-natural, synthetic “artificial” methods such as, for example, mutagenesis. Such methods have been described (U.S. Pat. No. 5,565,350; WO 00/15815; also see above).

[0434.1.8.10] to [0435.1.8.10] for the disclosure of these paragraphs see [0434.1.8.8] to [0435.1.8.8] above.

Transgenic plants comprising the respective fine chemical(s) synthesized in the process according to the invention can be marketed directly without isolation of the fine chemical synthesized. In the process according to the invention, plants are understood as meaning all plant parts, plant organs such as leaf, stalk, root, tubers or seeds or propagation material or harvested material or the intact plant. In this context, the seed encompasses all parts of the seed such as the seed coats, epidermal cells, seed cells, endosperm or embryonic tissue. The fine chemical produced in the process according to the invention may, however, also be isolated from the plant. In case of stearic acid this can be in free form or bound to proteins. Fine chemical(s) produced by this process can be harvested by harvesting the non-human organisms either from the culture in which they grow or from the field. For example, this can be done via squeezing, grinding and/or extraction, salt precipitation and/or ion-exchange chromatography of the plant parts, preferably the plant seeds, plant fruits, plant tubers and the like.

[0437.1.8.10] to [0454.1.8.10] for the disclosure of these paragraphs see [0437.1.8.8] to [0454.1.8.8] above above.

In one embodiment, the present invention relates to a method for the identification of a gene product conferring the production of or an increase in the fine chemical production in a non-human cell, comprising the following steps:
(a) contacting, e.g. hybridizing, a, some or all nucleic acid molecules of a sample, e.g. cells, tissues, plants or microorganisms or a nucleic acid library, which can contain a candidate gene encoding a gene product conferring the production of or an increase in the fine chemical after expression, with the nucleic acid molecule of the present invention as shown in the respective line in column 5 or 8 of Table I A or B of application no. 10, preferably the coding region thereof, or a fragment or homolog thereof;
(b) identifying the nucleic acid molecules, which hybridize under relaxed stringent conditions with the nucleic acid molecule of the present invention in particular to the nucleic acid molecule sequence shown in the respective line in Table I, application no. 10, columns 5 or 8, preferably the coding region thereof, of a fragment or a homolog thereof, preferably in Table I B, application no. 10, columns 5 or 8, preferably the coding region thereof, of a fragment or a homolog thereof, and, optionally, isolating the full length cDNA clone or complete genomic clone;
(c) introducing the candidate nucleic acid molecules or a fragment thereof in host cells, preferably in a plant cell or a microorganism, appropriate for producing the fine chemical;
(d) expressing the identified nucleic acid molecules in the host cells for which the production of or the increased production of the fine chemical is desired;
(e) assaying the fine chemical level in the non-human host cells; and
(f) identifying the nucleic acid molecule and/or its gene product which expression confers the production of or an increase in the fine chemical level in the non-human host cell after expression compared to the wild type.

for the disclosure of this paragraph see [0456.1.8.8] above.

In another embodiment, the present invention relates to a method for the identification of a gene product conferring the production of or an increase in the fine chemical production in a cell, comprising the following steps:
(a) identifying a nucleic acid molecule of a non-human organism, which is at least 20%, 25%, 30%, 35%, 40%, 50%, 60%, 70%, 80%, 90%, 95%, 96%, 97%, 98%, 99%, 99.5% or more homolog to the nucleic acid molecule encoding a polypeptide comprising the polypeptide molecule as shown in the respective line in column 5 or 8 of Table II, application no. 10, or comprising a consensus sequence or a polypeptide motif as shown in the respective line in column 8 of Table IV, application no. 10, or being encoded by a nucleic acid molecule comprising a polynucleotide as shown in the respective line in column 5 or 8 of Table I, application no. 10, preferably the coding region thereof, or a homologue thereof as described herein, for example via homology search in a data bank;
(b) introducing the candidate nucleic acid molecule in non-human host cells, preferably in a plant cell or a microorganism, appropriate for producing the fine chemical;
(c) expressing the identified nucleic acid molecule in the non-human host cells;
(d) assaying the fine chemical level in the non-human host cells; and
(e) identifying the nucleic acid molecule and its gene product which expression confers the production of or an increase in the fine chemical level in the non-human host cell after expression compared to the wild type.

for the disclosure of this paragraph see [0458.1.8.8] above.

Further, the nucleic acid molecules disclosed herein, in particular the nucleic acid molecules shown in the respective line in column 5 or 8 of Table I A or B of application no. 10, may be sufficiently homologous to the sequences of related species such that these nucleic acid molecules may serve as markers for the construction of a genomic map in related organism or for association mapping. Furthermore natural variation in the genomic regions corresponding to nucleic acids disclosed herein, in particular the nucleic acid molecule shown in the respective line in column 5 or 8 of Table I A or B, of application no. 10 or homologous thereof, may lead to variation in the activity of the proteins disclosed herein, in particular the proteins comprising polypeptides as shown in the respective lines in column 5 or 8 of Table II A or B, of application no. 10 or their homologous, or comprising the consensus sequence or the polypeptide motif as shown in the respective line in column 8 of Table IV, and in consequence in a natural variation of an increased fine chemical production.

In consequence natural variation eventually also exists in form of more active allelic variants leading already to a relative increase in the fine chemical. Different variants of the nucleic acid molecules disclosed herein, in particular the nucleic acid molecules comprising the nucleic acid molecules as shown in the respective line in column 5 or 8 of Table I A or B of application no. 10, preferably the coding region thereof, which corresponds to different levels of increase in fine chemical, e.g. different levels of increase in fine chemical, can be indentified and used for marker assisted breeding for an enhanced production of the fine chemical.

Natural variation may also exist in the regulatory regions, e.g. the promoters of the natural genes comprising the nucleic acid molecules as shown in the respective line column 5 or 8 of Table I A or B of application no. 10, preferably the coding region thereof, which can similarly correspond to different levels of increase in the fine chemical, e.g. different levels of increase in the fine chemical can be identified and used for marker assisted breeding for an enhanced production of the fine chemical.

Accordingly, the present invention relates to a method for breeding plants for the production of the fine chemical, comprising
(a) selecting a first plant variety with an increased production of the fine chemical, e.g. based on an increased expression of a nucleic acid molecule of the invention as disclosed herein, in particular of a nucleic acid molecule comprising a nucleic acid molecule as shown in the respective line in column 5 or 8 of Table I A or B of application no. 10, preferably the coding region thereof, or fragments or homologs thereof, or a polypeptide comprising a polypeptide as shown in the respective line in column 5 or 8 of Table II A or B of application no. 10, or fragments or homologs thereof, or comprising a consensus sequence or a polypeptide motif as shown in the respective line in column 8 of Table IV, or a homolog thereof, as described herein;
(b) associating the level of increased production of the fine chemical, with the expression level or the genomic structure of a gene encoding said polypeptide or said nucleic acid molecule;
(c) crossing the first plant variety with a second plant variety, which significantly differs in its level of fine chemical; and
(d) identifying, which of the offspring varieties has got increased production of the fine chemical by the expression level of said polypeptide or nucleic acid molecule or the genomic structure of the gene(s) encoding said polypeptide or nucleic acid molecule of the invention.

[0462.1.8.10] to [0482.1.8.10] for the disclosure of these paragraphs see [0462.1.8.8] to [0482.1.8.8] above.

In a further embodiment the present invention is characterized by the features as disclosed in the following items:

### Items:

Item 1. A process for the production of the fine chemical stearic acid
(a) increasing or generating one or more activities selected from the group consisting of 2-C-methyl-D-erythritol 4-phosphate cytidylyltransferase, 3-deoxy-7-phosphoheptulonate synthase, 3-ketoacyl-CoA thiolase, acetyl-coenzyme A synthetase, acid shock protein, aconitate hydratase, arginine decarboxylase, aspartate-ammonia ligase, At2g45420-protein, ATP-binding component of a transport system, b0283-protein, b0371-protein, b1672-protein, b2032-protein, b2399-protein, b2513-protein, b2849-protein, b3989-protein, b4121-protein, betaine aldehyde dehydrogenase, calcium-dependent protein kinase, cation-transporting ATPase, CCAAT-binding transcription factor, citrate synthase, coproporphyrinogen III oxidase, CTP synthetase, dihydroxyacetone kinase, ethanolamine utilization protein, F-box protein, glucokinase, glucose-6-phosphate 1-dehydrogenase, glutaredoxin, glycerol-3-phosphate dehydrogenase, heat shock transcription factor, isocitrate dehydrogenase, lipoprotein precursor, monothiol glutaredoxin, NADH dehydrogenase subunit N, oxidoreductase, phospho-2-dehydro-3-deoxyheptonate aldolase, protease, protein phosphatase, pyruvate kinase, sll1032-protein, sodium/proton antiporter, transcription factor, tryptophan synthase alpha chain, ycl049c-protein, ydr507c-protein, YGR104C-protein, and yor392w-protein, in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stearic acid or a composition comprising stearic acid in said non-human organism or in the culture medium surrounding said non-human organism.

Item 2. A process for the production of the fine chemical stearic acid, which comprises
(A)
(i) increasing or generating of the expression of; and/or
(ii) increasing or generating the expression of an expression product of; and/or
(iii) increasing or generating one or more activities of an expression product encoded by;
at least one respective nucleic acid molecule comprising a nucleic acid molecule selected from the group consisting of:
(a) a nucleic acid molecule encoding the polypeptide shown in column 5 or 8 of Table II, preferably Table II B, application no.10, or a homolog or a fragment thereof;
(b) a nucleic acid molecule shown in column 5 or 8 of Table I, preferably Table I B, application no.10, or a homolog or a fragment thereof (preferably the coding region thereof);
(c) a nucleic acid molecule, which, as a result of the degeneracy of the genetic code, can be derived from a polypeptide sequence depicted in column 5 or 8 of Table II, preferably Table II B, application no.10;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 10, or the coding region thereof;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no.10;
(i) a nucleic acid molecule encoding a polypeptide having the activity represented by a respective protein as depicted in column 5 of Table II, application no. 10;
(j) a nucleic acid molecule which comprises a polynucleotide, which is obtained by amplifying a cDNA library or a genomic library using the primers as depicted in column 8 of Table III, application no. 10; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto;
in a non-human organism or a part thereof, preferably a microorganism, a plant cell, a plant or a part thereof, as compared to a corresponding non-transformed wild type non-human organism or a part thereof; and
(B) growing the non-human organism or a part thereof under conditions which permit the production of stearic acid or a composition comprising stearic acid in said non-human organism or in the culture medium surrounding said non-human organism.

Item 3. A process of items 1 or 2, comprising of recovering stearic acid in its free or bound form.

Item 4. The process of any one of items 1 to 3, comprising the following steps:
(a) selecting a non-human organism or a part thereof expressing a polypeptide encoded by the nucleic acid molecule characterized in item 2;
(b) mutagenizing the selected non-human organism or the part thereof;
(c) comparing the activity or the expression level of said polypeptide in the mutagenized non-human organism or the part thereof with the activity or the expression of said polypeptide of the selected non-human organisms or the part thereof;
(d) selecting the mutated non-human organisms or parts thereof, which comprise an increased activity or expression level of said polypeptide compared to the selected non-human organism or the part thereof;
(e) optionally, growing and cultivating the non-human organisms or the parts thereof;
and
(f) recovering, and optionally isolating, the free or bound stearic acid produced by the selected mutated non-human organisms or parts thereof.

Item 5. The process of any one of items 1 to 4 wherein the activity of said protein or the expression of said nucleic acid molecule is increased or generated transiently or stably.

Item 6. An isolated nucleic acid molecule comprising a nucleic acid molecule selected from the group consisting of:
(a) a nucleic acid molecule encoding a polypeptide shown in Table II, application no. 10, column 5 or 8, preferably shown in Table II B, application no. 10, column 8, or a homolog or a fragment thereof;
(b) a nucleic acid molecule shown in Table I, application no. 10, column 5 or 8, preferably shown in Table I B, application no. 10, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 10, preferably in column 8 of Table II B, application no. application no.,
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 3, preferably shown in column 8 of Table I B, application no. 10, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 10;
(i) a nucleic acid molecule encoding a polypeptide having the activity represented by a respective protein as depicted in column 5 of Table II, application no. 10;
(j) a nucleic acid molecule comprising a polynucleotide which is obtained by amplifying a cDNA library or a genomic library using the primers in Table III, application no. 10, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (i),
or a nucleic acid molecule comprising a sequence which is complementary thereto.

Item 7. A nucleic acid construct which confers the expression of the nucleic acid molecule as defined in item 6, comprising one or more regulatory elements.

Item 8. An expression cassette comprising
(a) a promoter, preferably selected from the group consisting of Big35S, PCUbi, Super and USP;
(b) a nucleic acid molecule as defined in item 6.

Item 9. A vector comprising the nucleic acid molecule as defined in item 6 or the nucleic acid construct as claimed in item 7 or the expression cassette as claimed in item 8.

Item 10. A host cell or a plant organelle, which has been transformed with the vector as claimed in item 9 or the nucleic acid molecule as defined in item 6 or the nucleic acid construct as claimed in item 7 or the expression cassette as claimed in item 8.

Item 11. The host cell of item 10, which is a plant cell or a microorganism.

Item 12. A process for producing a polypeptide, wherein the polypeptide is expressed in a host cell as claimed in item 10 or 11.

Item 13. A polypeptide produced by the process as claimed in item 12 or encoded by the nucleic acid molecule as defined in item 6.

Item 14. An antibody, which binds specifically to the polypeptide as claimed in item 13.

Item 15. A plant tissue, propagation material, harvested material or a plant or a part thereof comprising the host cell as claimed in item 10 or 11.

Item 16. A process for the identification of a compound conferring an increase in stearic acid production in a non-human organism, comprising the steps:
(a) culturing a plant cell or tissue or microorganism or maintaining a plant expressing the polypeptide encoded by the nucleic acid molecule as defined in item 6 conferring an increase in the amount of stearic acid in a non-human organism or a part thereof and a readout system capable of interacting with the polypeptide under suitable conditions which permit the interaction of the polypeptide with said readout system in the presence of a compound or a sample comprising a plurality of compounds and capable of providing a detectable signal in response to the binding of a compound to said polypeptide under conditions which permit the expression of said readout system and of the polypeptide encoded by the nucleic acid molecule of item 6 conferring an increase in the amount of stearic acidin a non-human organism or a part thereof;
(b) identifying if the compound is an effective agonist by detecting the presence or absence or increase of a signal produced by said readout system.

Item 17. A method for the production of an agricultural composition comprising the steps of the method of item 16 and formulating the compound identified in item 16 in a form acceptable for an application in agriculture.

Item 18. A composition comprising the nucleic acid molecule as defined in item 6, the polypeptide as claimed in item 13, the nucleic acid construct as claimed in item 7, the expression cassette as claimed in item 8, the vector as claimed in item 9, the compound as claimed in item 16, the antibody as claimed in item 14, and optionally an agricultural acceptable carrier.

Item 19. Use of the nucleic acid molecule as defined in item 6 for the identification of a nucleic acid molecule conferring an increase in stearic acid after expression.

Item 20. Cosmetic, pharmaceutical, food or feed composition comprising the nucleic acid molecule as defined in item 6, the polypeptide as claimed in item 13, the nucleic acid construct as claimed in item 7, the expression cassette as claimed in item 8, the vector as claimed in item 9, the antibody as claimed in item 14 , the plant or a part thereof, or plant tissue as claimed in item 15, the harvested material or propagation material as claimed in item 15 or the host cell of item as claimed in items 10 or 11.

Item 21. Use of the nuclei acid molecule as defined in item 6, the polypeptide as claimed in item 13, the nucleic acid construct as claimed in item 7, the expression cassette as claimed in item 8, the vector as claimed in claim9, the plant or plant tissue as claimed in item 15, or the host cell of item 10 to 11 for the production of plant resistant to a herbicide inhibiting the production of stearic acid.

[0483.1.8.10] to [0494.1.8.10] for the disclosure of these paragraphs see [0483.1.8.8] to [0494.1.8.8] above.

### Example 11a

### PCR Amplification of the Sequences

Unless otherwise specified, standard methods as described in Sambrook et al., Molecular Cloning: A laboratory manual, Cold Spring Harbor 1989, Cold Spring Harbor Laboratory Press are used.

The inventive sequences as shown in the respective line in Table I, column 5, preferably the coding region thereof, were amplified by PCR as described in the protocol of the Pfu Ultra, Pfu Turbo or Herculase DNA polymerase (Stratagene). The composition for the protocol of the Pfu Ultra, Pfu Turbo or Herculase DNA polymerase was as follows: 1×PCR buffer (Stratagene), 0.2 mM of each dNTP, 100 ng genomic DNA of Saccharomyces cerevisiae (strain S288C; Research Genetics, Inc., now Invitrogen), Escherichia coli (strain MG1655; E. coli Genetic Stock Center), Synechocystis sp. (strain PCC6803), Azotobacter vinelandii (strain N.R. Smith,16), Thermus thermophilus (HB8) or 50 ng cDNA from various tissues and development stages of Arabidopsis thaliana (ecotype Columbia), Physcomitrella patens, Glycine max (variety Resnick), Brassica napus, Oryza sativa or Zea mays (variety B73, Mo17, A188), 50 pmol forward primer, 50 pmol reverse primer, with or without 1 M Betaine, 2.5 u Pfu Ultra, Pfu Turbo or Herculase DNA polymerase.

The amplification cycles were as follows:

1 cycle of 2-3 minutes at 94-95° C., then 25-36 cycles with 30-60 seconds at 94-95° C., 30-45 seconds at 50-60° C. and 210-480 seconds at 72° C., followed by 1 cycle of 5-10 minutes at 72° C., then 4-16° C.—preferably for Saccharomyces cerevisiae, Escherichia coli, Synechocystis sp., Azotobacter vinelandii, Thermus thermophilus.

In case of Arabidopsis thaliana, Brassica napus, Glycine max, Oryza sativa, Physcomitrella patens, Zea mays the amplification cycles were as follows:

1 cycle with 30 seconds at 94° C., 30 seconds at 61 ° C., 15 minutes at 72° C., then 2 cycles with 30 seconds at 94° C., 30 seconds at 60° C., 15 minutes at 72° C., then 3 cycles with 30 seconds at 94° C., 30 seconds at 59° C., 15 minutes at 72° C., then 4 cycles with 30 seconds at 94° C., 30 seconds at 58° C., 15 minutes at 72° C., then 25 cycles with 30 seconds at 94° C., 30 seconds at 57° C., 15 minutes at 72° C., then 1 cycle with 10 minutes at 72° C., then finally 4-16° C.

RNAs were generated with the RNeasy Plant Kit according to the standard protocol (Qiagen) and Superscript II Reverse Transkriptase was used to produce double stranded cDNA according to the standard protocol (Invitrogen).

ORF specific primer pairs for the genes to be expressed are shown in the respective line in Table III, column 8. The following adapter sequences were added to Saccharomyces cerevisiae ORF specific primers (see Table III) for cloning purposes:

SEQ ID NO: 20

| | | |
|---|---|---|
| i) | foward primer: | 5′-GGAATTCCAGCTGACCACC-3′ |

SEQ ID NO: 21

| | | |
|---|---|---|
| ii) | reverse primer: | 5′-GATCCCCGGGAATTGCCATG-3′ |

These adaptor sequences allow cloning of the ORF into the various vectors containing the Resgen adaptors, see table column E of Table c.

The following adapter sequences were added to Saccharomyces cerevisiae, Escherichia coli, Synechocystis sp., Azotobacter vinelandii, Thermus thermophilus, Arabidopsis thaliana, Brassica napus, Glycine max, Oryza sativa , Physcomitrella patens, or Zea mays ORF specific primers for cloning purposes:

| SEQ ID NO: 22 | | |
|---|---|---|
| iii) | forward primer: | 5′-TTGCTCTTCC-3′ |
| SEQ ID NO: 23 | | |
| iiii) | reverse primer: | 5′-TTGCTCTTCG-3′ |

The adaptor sequences allow cloning of the ORF into the various vectors containing the Colic adaptors, see table column E of Table c.

Therefore for amplification and cloning of Saccharomyces cerevisiae SEQ ID NO: 66772, a primer consisting of the adaptor sequence i) and the ORF specific sequence SEQ ID NO: 67180 and a second primer consisting of the adaptor sequence ii) and the ORF specific sequence SEQ ID NO: 67181 were used.

For amplification and cloning of Escherichia coli SEQ ID NO: 7333, a primer consisting of the adaptor sequence iii) and the ORF specific sequence SEQ ID NO: 7679 and a second primer consisting of the adaptor sequence iiii) and the ORF specific sequence SEQ ID NO: 7680 were used.

For amplification and cloning of Synechocystis sp. SEQ ID NO: 11471, a primer consisting of the adaptor sequence iii) and the ORF specific sequence SEQ ID NO: 11983 and a second primer consisting of the adaptor sequence iiii) and the ORF specific sequence SEQ ID NO: 11984 were used.

For amplification and cloning of Arabidopsis thaliana SEQ ID NO: 1061, a primer consisting of the adaptor sequence iii) and the ORF specific sequence SEQ ID NO: 1293 and a second primer consisting of the adaptor sequence iiii) and the ORF specific sequence SEQ ID NO: 1294 were used.

For amplification and cloning of Brassica napus SEQ ID NO: 15532, a primer consisting of the adaptor sequence iii) and the ORF specific sequence SEQ ID NO: 16150 and a second primer consisting of the adaptor sequence iiii) and the ORF specific sequence SEQ ID NO: 16151 were used.

For amplification and cloning of Glycine max SEQ ID NO: 10811, a primer consisting of the adaptor sequence iii) and the ORF specific sequence SEQ ID NO: 11207 and a second primer consisting of the adaptor sequence iiii) and the ORF specific sequence SEQ ID NO: 11208 were used.

Following these examples every sequence disclosed in Table I, preferably column 5, especially the coding region thereof can be cloned by fusing the adaptor sequences to the respective specific primers sequences as disclosed in Table III, column 8 using the respective vectors shown in Table c.

[0496.1.8.10] to [0499.1.8.10] for the disclosure of these paragraphs see [0496.1.8.8] to [0499.1.8.8] above.

### Example 11e

### Cloning of Inventive Sequences as Shown in Table I, Column 5 in the Different Expression Vectors

For cloning of for example SEQ ID NO: 7333 from Escherichia coli or any other ORF from Escherichia coli, Synechocystis sp., Arabidopsis thaliana, Brassica napus, or Glycine max the vector DNA was treated with the restriction enzymes Pacl and Ncol following the standard protocol (MBI Fermentas).

For cloning for example the ORFs of SEQ ID NO: 66772 from Saccharomyces cerevisiae or any other ORF from Saccharomyces cerevisiae into vectors containing the Resgen adaptor sequence the respective vector DNA was treated with the restriction enzyme Ncol.

For cloning of for example ORFs SEQ ID NO: 117482 from Saccharomyces cerevisiae or any other ORF from Saccharomyces cerevisiae into vectors containing the Colic adaptor sequence, the respective vector DNA was treated with the restriction enzymes Pacl and Ncol following the standard protocol (MBI Fermentas).

In all cases the reaction was stopped by inactivation at 70° C. for 20 minutes and purified over QlAquick or NucleoSpin Extract II columns following the standard protocol (Qiagen or Macherey-Nagel).

Then the PCR-product representing the amplified ORF with the respective adapter sequences and the vector DNA were treated with T4 DNA polymerase according to the standard protocol (MBI Fermentas) to produce single stranded overhangs with the parameters 1 unit T4 DNA polymerase at 37° C. for 2-10 minutes for the vector and 1-2 u T4 DNA polymerase at 15-17° C. for 10-60 minutes for the PCR product representing SEQ ID NO: 7333.

The reaction was stopped by addition of high-salt buffer and purified over QlAquick or NucleoSpin Extract II columns following the standard protocol (Qiagen or Macherey-Nagel).

According to this example the skilled person is able to clone all sequences disclosed in Table I, preferably column 5 or column 8, especially the coding region thereof.

Approximately 30-60 ng of prepared vector and a defined amount of prepared amplificate were mixed and hybridized at 65° C. for 15 minutes followed by 37° C. 0.1 ° C/1 seconds, followed by 37° C. 10 minutes, followed by 0.1 ° C/1 seconds, then 4-10 ° C.

The ligated constructs were transformed in the same reaction vessel by addition of competent E. coli cells (strain DHSalpha) and incubation for 20 minutes at 1 ° C. followed by a heat shock for 90 seconds at 42° C. and cooling to 1-4° C. Then, complete medium (SOC) was added and the mixture was incubated for 45 minutes at 37° C. The entire mixture was subsequently plated onto an agar plate with 0.05 mg/ml kanamycin and incubated overnight at 37° C.

The outcome of the cloning step was verified by amplification with the aid of primers which bind upstream and downstream of the integration site, thus allowing the amplification of the insertion. The amplifications were carried out as described in the protocol of Taq DNA polymerase (Gibco-BRL).

The amplification cycles were as follows:

1 cycle of 1-5 minutes at 94° C., followed by 35 cycles of in each case 15-60 seconds at 94° C., 15-60 seconds at 50-66° C. and 5-15 minutes at 72° C., followed by 1 cycle of 10 minutes at 72° C., then 4-16° C.

Several colonies were checked, but only one colony for which a PCR product of the expected size was detected was used in the following steps.

A portion of this positive colony was transferred into a reaction vessel filled with complete medium (LB) supplemented with kanamycin and incubated overnight at 37° C.

The plasmid preparation was carried out as specified in the Qiaprep or NucleoSpin Multi-96 Plus standard protocol (Qiagen or Macherey-Nagel).

[0501.1.8.10] to [0503.1.8.10] for the disclosure of these paragraphs see [0501.1.8.8] to [0503.1.8.8] above.

Table d showing results of plant analyses

TABLE d

| SeqID | Target | Locus | Metabolite | Source | Promotor | Method | Min | Max |
|---|---|---|---|---|---|---|---|---|
| 1061 | non- | At1g68320 | stearic | ARA_LEAF | p-PcUBI | GC | 17 | 84 |
| | targeted | | acid | | | | | |
| 1298 | non- | At2g25070 | stearic | ARA_LEAF | p-PcUBI | GC | 18 | 44 |
| | targeted | | acid | | | | | |
| 120810 | plastidic | At2g44350 | stearic | ARA_LEAF | p-Super | GC | 17 | 25 |
| | | | acid | | | | | |
| 1623 | non- | At2g45420 | stearic | ARA_LEAF | p-PcUBI | GC | 17 | 691 |
| | targeted | | acid | | | | | |
| 120952 | non- | At3g16820 | stearic | ARA_LEAF | p-PcUBI | GC | 20 | 28 |
| | targeted | | acid | | | | | |
| 22921 | non- | At3g20910 | stearic | ARA_LEAF | p-PcUBI | GC | 28 | 60 |
| | targeted | | acid | | | | | |
| 2935 | non- | At3g62950 | stearic | ARA_LEAF | p-PcUBI | GC | 23 | 58 |
| | targeted | | acid | | | | | |
| 23482 | non- | At4g15660 | stearic | ARA_LEAF | p-PcUBI | GC | 19 | 79 |
| | targeted | | acid | | | | | |
| 3279 | non- | At4g15670 | stearic | ARA_LEAF | p-PcUBI | GC | 15 | 89 |
| | targeted | | acid | | | | | |
| 24232 | non- | At4g18880 | stearic | ARA_LEAF | p-PcUBI | GC | 20 | 28 |
| | targeted | | acid | | | | | |
| 68849 | non- | At4g26080 | stearic | ARA_LEAF | p-PcUBI | GC | 17 | 20 |
| | targeted | | acid | | | | | |
| 4102 | non- | At4g33040 | stearic | ARA_LEAF | p-PcUBI | GC | 21 | 40 |
| | targeted | | acid | | | | | |
| 4348 | non- | At4g35310 | stearic | ARA_LEAF | p-PcUBI | GC | 99 | 132 |
| | targeted | | acid | | | | | |
| 25498 | non- | At5g59220 | stearic | ARA_LEAF | p-PcUBI | GC | 15 | 38 |
| | targeted | | acid | | | | | |
| 121076 | plastidic | B0283 | stearic | ARA_LEAF | p-Super | GC | 19 | 29 |
| | | | acid | | | | | |
| 121111 | non- | B0312 | stearic | ARA_SEED_2 | p-USP | GC | 12 | 21 |
| | targeted | | acid | | | | | |
| 121637 | non- | B0371 | stearic | ARA_LEAF | p-Super | GC | 26 | 60 |
| | targeted | | acid | | | | | |
| 7333 | non- | B0449 | stearic | ARA_LEAF | p-Super | GC | 18 | 75 |
| | targeted | | acid | | | | | |
| 37400 | non- | B1186 | stearic | ARA_LEAF | p-Super | GC | 15 | 91 |
| | targeted | | acid | | | | | |
| 38266 | plastidic | B1431 | stearic | ARA_LEAF | p-Super | GC | 19 | 31 |
| | | | acid | | | | | |
| 38300 | non- | B1597 | stearic | ARA_LEAF | p-Super | GC | 22 | 178 |
| | targeted | | acid | | | | | |
| 78771 | non- | B1672 | stearic | ARA_LEAF | p-Super | GC | 17 | 30 |
| | targeted | | acid | | | | | |
| 38573 | non- | B1845 | stearic | ARA_LEAF | p-Super | GC | 22 | 31 |
| | targeted | | acid | | | | | |
| 8920 | non- | B2032 | stearic | ARA_LEAF | p-Super | GC | 18 | 89 |
| | targeted | | acid | | | | | |
| 39237 | non- | B2399 | stearic | ARA_LEAF | p-Super | GC | 18 | 63 |
| | targeted | | acid | | | | | |
| 115628 | non- | B2439 | stearic | ARA_LEAF | p-Super | GC | 16 | 24 |
| | targeted | | acid | | | | | |
| 79509 | non- | B2482 | stearic | ARA_LEAF | p-Super | GC | 19 | 73 |
| | targeted | | acid | | | | | |
| 9167 | non- | B2513 | stearic | ARA_LEAF | p-Super | GC | 16 | 121 |
| | targeted | | acid | | | | | |
| 121657 | plastidic | B2601 | stearic | ARA_SEED_2 | p-USP | GC | 11 | 51 |
| | | | acid | | | | | |
| 81075 | non- | B2747 | stearic | ARA_LEAF | p-Super | GC | 18 | 44 |
| | targeted | | acid | | | | | |
| 81980 | non- | B2849 | stearic | ARA_LEAF | p-Super | GC | 21 | 129 |
| | targeted | | acid | | | | | |
| 83657 | plastidic | B3744 | stearic | ARA_SEED_2 | p-USP | GC | 9 | 27 |
| | | | acid | | | | | |
| 100675 | plastidic | B3845 | stearic | ARA_SEED_2 | p-USP | GC | 10 | 27 |
| | | | acid | | | | | |
| 44372 | non- | B3989 | stearic | ARA_LEAF | p-Super | GC | 18 | 35 |
| | targeted | | acid | | | | | |
| 44609 | non- | B4121 | stearic | ARA_LEAF | p-Super | GC | 18 | 35 |
| | targeted | | acid | | | | | |
| 10811 | non- | GM02LC12622 | stearic | ARA_LEAF | p-PcUBI | GC | 17 | 99 |
| | targeted | | acid | | | | | |
| 46751 | non- | GM02LC19289 | stearic | ARA_LEAF | p-PcUBI | GC | 17 | 41 |
| | targeted | | acid | | | | | |
| 85510 | plastidic | Sll0593 | stearic | ARA_LEAF | p-PcUBI | GC | 17 | 54 |
| | | | acid | | | | | |
| 85723 | mitochondrial | Sll1032 | stearic | ARA_LEAF | p-PcUBI | GC | 17 | 22 |
| | | | acid | | | | | |
| 53878 | mitochondrial | Sll1443 | stearic | ARA_LEAF | p-PcUBI | GC | 17 | 42 |
| | | | acid | | | | | |
| 11471 | mitochondrial | Sll1917 | stearic | ARA_LEAF | p-PcUBI | GC | 17 | 37 |
| | | | acid | | | | | |
| 55385 | mitochondrial | Sll1920 | stearic | ARA_LEAF | p-PcUBI | GC | 15 | 34 |
| | | | acid | | | | | |
| 11990 | plastidic | Slr0338 | stearic | ARA_LEAF | p-PcUBI | GC | 20 | 36 |
| | | | acid | | | | | |
| 87655 | plastidic | Slr0966 | stearic | ARA_LEAF | p-PcUBI | GC | 27 | 48 |
| | | | acid | | | | | |
| 58324 | non- | Slr1312 | stearic | ARA_LEAF | p-PcUBI | GC | 16 | 70 |
| | targeted | | acid | | | | | |
| 121908 | non- | Yal054c | stearic | ARA_LEAF | p-PcUBI | GC | 20 | 28 |
| | targeted | | acid | | | | | |
| 88879 | plastidic | Ybr249c | stearic | ARA_LEAF | p-Super | GC | 20 | 109 |
| | | | acid | | | | | |
| 113220 | plastidic | Ycl049c | stearic | ARA_LEAF | p-Super | GC | 11 | 17 |
| | | | acid | | | | | |
| 119408 | non- | Ydl022w | stearic | ARA_SEED_2 | p-PcUBI | GC | 17 | 35 |
| | targeted | | acid | | | | | |
| 122228 | non- | Ydl066w | stearic | ARA_SEED_2 | p-PcUBI | GC | 9 | 24 |
| | targeted | | acid | | | | | |
| 90165 | non- | Ydr507c | stearic | ARA_LEAF | Big35S | GC | 16 | 51 |
| | targeted | | acid | | | | | |
| 120309 | non- | Yfl053w | stearic | ARA_LEAF | Big35S | GC | 19 | 37 |
| | targeted | | acid | | | | | |
| 117482 | non- | Ygr104c | stearic | ARA_SEED_2 | p-PcUBI | GC | 14 | 20 |
| | targeted | | acid | | | | | |
| 66772 | non- | Ylr304c | stearic | ARA_LEAF | Big35S | GC | 16 | 35 |
| | targeted | | acid | | | | | |
| 15183 | plastidic | Yor392w | stearic | ARA_LEAF | p-Super | GC | 20 | 32 |
| | | | acid | | | | | |
| 15532 | plastidic | 51340801_CANOLA | stearic | ARA_LEAF | p-PcUBI | GC | 16 | 58 |
| | | | acid | | | | | |
| 16883 | plastidic | 59582753_SOYBEAN | stearic | ARA_LEAF | p-PcUBI | GC | 16 | 42 |
| | | | acid | | | | | |

Column 1 shows the SEQ ID NO, Column 2 shows the expression type (targeted or non-targeted), Column 3 shows the “gene name” (locus), Column 4 shows the metabolite analyzed, Column 5 indicates the A. thaliana source tissue analyzed, Column 6 indicates the used promoter for expression, Column 7 indicates the analytical method. Columns 8 and 9 show the minimum and the maximum increase of the analyzed metabolite (in percent) in comparison to the wild type (=ratio_by_weight, given as percent increase).

When the analyses were repeated independently, all results proved to be significant.

[0505.1.8.10] to [0515.1.8.10] for the disclosure of these paragraphs see [0505.1.8.8] to [0515.1.8.8] above.

In a further embodiment, the present invention relates in paragraphs [0000.1.8.11] to [0514.1.8.11] to a further process for the production of the fine chemical palmitic acid as defined below and corresponding embodiments as described herein as follows.

[0001.1.8.11] to [0002.1.8.11] for the disclosure of these paragraphs see [0001.1.8.8] to [0002.1.8.8] above.

Palmitic acid is a major component for manufacturing of soaps, lubricating oils and waterproofing materials. Furhermore it is used for the synthesis of metallic palmitates. Additional applications are as food additive and in the synthesis of food-grade additives; as a constituent of cosmetic formulations. Palmitic acid is a major component of many natural fats and oils in the form of a glyceryl ester, e.g. palm oil, and in most commercial-grade stearic acid products.

[0004.1.8.11] to [0005.1.8.11] for the disclosure of these paragraphs see [0004.1.8.8] to [0005.1.8.8] above.

Palmitic acid is as mentioned above the major fat in meat and dairy products.

Further uses or palmitic acid are as food ingredients raw material for emulsifiers or personal care emulsifier for facial creams and lotions.

Palmitic acid is also used in shaving cream formulations, waxes or fruit wax formulations.

[0009.1.11.11] to [0011.1.8.11] for the disclosure of these paragraphs see [0009.1.8.8] to [00011.1.8.8] above.

It is an object of the present invention to develop an inexpensive process for the synthesis of palmitic acid.

for the disclosure of this paragraph see [0013.1.8.8] above.

Accordingly, in a first embodiment, the invention relates to a process for the production of at least one fine chemical selected from the group consisting of: palmitic acid, or, in other words, of the “fine chemical” or “fine chemical of the invention”.

The terms “fine chemical of the invention”, “fine chemical” or “the fine chemical” are used herein equally and relate in context of the paragraphs or sections [0014.1.11.11] to [0514.1.8.11] essentially to the metabolite or the metabolites indicated in column 7, application no. 11 of Tables I, II and IV in the respective line.

Further, the term “in context of any of the paragraphs [0014.1.11.11] to [0514.1.8.11]” as used herein means that for any of said paragraphs [0014.1.11.11] to [0514.1.8.11] the term “the fine chemical” is understood to follow the definition of paragraphs or sections [0014.1.11.11] and [0015.1.11.11], independently whether it refers to any other paragraph or not and whether the reference recites the term “fine chemical” in an other context.

Thus, in cases where one or more paragraphs or sections are incorporated by reference into any of the present paragraphs [0014.1.11.11] to [0514.1.8.11], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to the definition of paragraph [0014.1.11.11] and [0015.1.11.11].

Accordingly, in one embodiment, the term “fine chemical of the invention”, “fine chemical” or “the fine chemical” as used herein (alone or in combination, like in FCRP) means “palmitic acid in context of the nucleic acid or polypeptide sequences listed in the respective same line of any one of Tables I to IV of application no. 11 and indicating in column 7 the metabolite “palmitic acid”.

In one embodiment, the term palmitic acid or the term “fine chemical” mean in context of the paragraphs or sections [0014.1.11.11] to [0514.1.8.11] at least one chemical compound with an activity of the above mentioned palmitic acid, respectively.

Accordingly, in one embodiment, the terms “fine chemical of the invention”, “fine chemical” or “the fine chemical” mean in context of any of the paragraphs [0014.1.11.11] to [0514.1.8.11] palmitic acid, its tryglycerides, lipids, oils and/or fats in free form or bound form. In a preferred embodiment, the term “the fine chemical” means palmitic acid in free form or e.g. in form of its tryglycerides, lipids, oils and/or fats. In a preferred embodiment “palmitic acid” means palmitic acid in free form.

In one embodiment, the term “the fine chemical” or “the respective fine chemical” mean in context of any of the paragraphs [0014.1.11.11] to [0514.1.8.11] palmitic acid. and/or tryglycerides, lipids, oils and/or fats of palmitic acid. and its salts, ester, thioester or palmitic acid in free form or bound to other compounds such as triglycerides, glycolipids, phospholipids etc. In a preferred embodiment, the term “the fine chemical” means palmitic acid, in free form or its salts or bound to triglycerides. Triglycerides, lipids, oils, fats or lipid mixture thereof shall mean any triglyceride, lipid, oil and/or fat containing any bound or free palmitic acid for example sphingolipids, phosphoglycerides, lipids, glycolipids such as glycosphingolipids, phospholipids such as phosphatidylethanolamine, phosphatidylcholine, phosphatidylserine, phosphatidylglycerol, phosphatidylinositol or diphosphatidylglycerol, or as monoacylglyceride, diacylglyceride or triacylglyceride or other fatty acid esters such as acetyl-Coenzym A thioester, which contain further saturated or unsaturated fatty acids in the fatty acid molecule.

In one embodiment, the term “the fine chemical” and the term “the respective fine chemical” mean at least one chemical compound with an activity of the abovementioned fine chemical. Further, the term “in context of any of the paragraphs [0014.1.11.11] to [0514.1.8.11]” as used herein means that for any of said paragraphs [0014.1.11.11] to [0514.1.8.11] the term “the fine chemical” is understood to follow the definition of section [0014.1.11.11] or section [0015.1.11.11], independently whether it refers to any other paragraph or not and whether the reference recites the term “fine chemical” in an other context.

Thus, in cases where one or more paragraphs or sections are incorporated by reference into any of the present paragraphs [0014.1.11.11] to [0514.1.8.11], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to this definition of this paragraph [0015.1.11.11].

Further, the term “fine chemicals” as used herein relates to compositions comprising said fine chemical(s), i.e. comprising palmitic acid, respectively.

Accordingly, the present invention relates to a process for the production of palmitic acid, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of chlorophyllase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of At1g19800-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of At1g23110-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of At2g45420-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of CCAAT-binding transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of ThiF family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of recombinase A in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of NADH-quinone oxidoreductase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of HesB/YadR/YfhF family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of peptidyl-prolyl cis-trans isomerase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of purine nucleoside phosphorylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of b0050-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of sodium/proton antiporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of b1234-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of b1248-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of b1330-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of lipoprotein precursor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of Fe—S subunit of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of acid shock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of b1670-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of b1837-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of b2032-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of colanic acid biosynthesis protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of b2099-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of b2107-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of b2399-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of ethanolamine utilization protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of b2682-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of 2-C-methyl-D-erythritol 4-phosphate cytidylyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of flavodoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of AAS bifunctional protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of b2849-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of b3814-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of threonine efflux protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of 3-ketoacyl-CoA thiolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of bifunctional protein (phosphoribosyltransferase and regulatory protein) in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of branched chain amino acid ABC transporter ATP-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of carbon dioxide concentrating mechanism protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of sll1032-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of phosphoribosylformyl glycinamidine synthase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of nitrate/nitrite transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of carbamoyl-phosphate synthase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of glutathione S-transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of ABC metal ion transporter substrate-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of acyl transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of coproporphyrinogen III oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of cation-transporting ATPase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of permease protein of ABC transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of aromatic acid decarboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of phosphate import ATP-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of arginine decarboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of phosphoadenosine phosphosulfate reductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of threonine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of metal-dependent hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of homocitrate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of acid phosphatase precursor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of glycerol-3-phosphate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of F-box protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of DnaJ-like chaperone in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of aconitate hydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of ymr013c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of ATPase epsilon subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of 49747384_SOYBEAN-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating one or more activities selected from the group consisting of glucose-6-phosphate 1-dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

An embodiment of the present invention relates to a process for the production of palmitic acid, which comprises
(a1) increasing or generating the activity of a polypeptide comprising a polypeptide as depicted in a respective line in column 5 or 8 of Table II, application no. 11, or a homolog or a fragment thereof, a consensus sequence or at least one polypeptide motif as depicted in the respective line in column 8 of Table IV, application no. 11; or
(a2) increasing or generating the activity of an expression product of one or more nucleic acid molecule(s) comprising a polynucleotide as depicted in the respective line in column 5 or 8 of Table I, application no. 11, preferably the coding region thereof, or a homolog or a fragment thereof; or
(a3) increasing or generating the activity of a functional equivalent of (a1) or (a2); in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of palmitic acid or a composition comprising palmitic acid in said non-human organism or in the culture medium surrounding said non-human organism.

Accordingly, the increase or generation of one or more said activities is for example conferred by one or more expression products of said nucleic acid molecule, e.g. proteins. Accordingly, in the present invention described above, the increase or generation of one or more said activities is for example conferred by one or more protein(s) each comprising a polypeptide selected from the group as depicted in Table II, application no. 11, column 5 and 8, or a homolog or a fragment thereof.

The process of the invention comprises in one embodiment the following steps:
(i) increasing or generating of the expression of; and/or
(ii) increasing or generating the expression of an expression product of; and/or
(iii) increasing or generating one or more activities of an expression product encoded by; at least one nucleic acid molecule (in the following “Fine Chemical Related Protein (FCRP)”encoding gene or “FCRP”-gene) comprising a nucleic acid molecule selected from the group consisting of:
(a) a nucleic acid molecule encoding the polypeptide shown in column 5 or 8 of Table II, preferably Table II B, application no. 11, or a homolog or a fragment thereof;
(b) a nucleic acid molecule shown in column 5 or 8 of Table I, preferably Table I B, application no. 11, or a homolog or a fragment thereof (preferably the coding region thereof);
(c) a nucleic acid molecule, which, as a result of the degeneracy of the genetic code, can be derived from a polypeptide sequence depicted in column 5 or 8 of Table II, preferably Table II B, application no. 11;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 11, or the coding region thereof;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d),
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 11;
(i) a nucleic acid molecule encoding a polypeptide having the activity represented by a respective protein as depicted in column 5 of Table II, application no. 11;
(j) a nucleic acid molecule which comprises a polynucleotide, which is obtained by amplifying a cDNA library or a genomic library using the primers as depicted in column 8 of Table III, application no. 11; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

The term “increasing or generating of the expression of” may also be interchanged by the term “overexpressing”.

In a preferred embodiment thereof said nucleic acid molecule encodes a polypeptide which has the activity of the polypeptide represented by a protein comprising a polypeptide as depicted in the corresponding hit in column 5 of Table II, application no. 11. In another preferred embodiment thereof said nucleic acid molecule confers a generation or an increase of the respective fine chemical in a non-human organism or a part thereof as compared to a corresponding wild-type non-human organism or part thereof.

In another preferred embodiment thereof said nucleic acid molecule encodes a polypeptide which has the activity of the polypeptide represented by a protein as depicted in the corresponding hit in column 5 of Table II, application no. 11, and confers a generation or an increase of the respective fine chemical in a non-human organism or a part thereof as compared to a corresponding wild-type non-human organism or part thereof.

Accordingly, the genes of the present invention or used in accordance with the present invention, which respectively encode a protein having an activity of 2-C-methyl-D-erythritol 4-phosphate cytidylyltransferase, 3-ketoacyl-CoA thiolase, 49747384_SOYBEAN-protein, AAS bifunctional protein, ABC metal ion transporter substrate-binding protein, acid phosphatase precursor, acid shock protein, aconitate hydratase, acyl transferase, arginine decarboxylase, aromatic acid decarboxylase, At1g19800-protein, At1g23110-protein, At2g45420-protein, ATPase epsilon subunit, b0050-protein, b1234-protein, b1248-protein, b1330-protein, b1670-protein, b1837-protein, b2032-protein, b2099-protein, b2107-protein, b2399-protein, b2682-protein, b2849-protein, b3814-protein, bifunctional protein (phosphoribosyltransferase and regulatory protein), branched chain amino acid ABC transporter ATP-binding protein, calcium-dependent protein kinase, carbamoyl-phosphate synthase subunit, carbon dioxide concentrating mechanism protein, cation-transporting ATPase, CCAAT-binding transcription factor, chlorophyllase, colanic acid biosynthesis protein, coproporphyrinogen III oxidase, DnaJ-like chaperone, ethanolamine utilization protein, F-box protein, Fe—S subunit of oxidoreductase, flavodoxin, glucose-6-phosphate 1-dehydrogenase, glutaredoxin, glutathione S-transferase, glycerol-3-phosphate dehydrogenase, heat shock transcription factor, HesB/YadR/YfhF family protein, homocitrate synthase, lipoprotein precursor, metal-dependent hydrolase, monothiol glutaredoxin, NADH-quinone oxidoreductase subunit, nitrate/nitrite transport protein, oxidoreductase, peptidyl-prolyl cis-trans isomerase, permease protein of ABC transporter, phosphate import ATP-binding protein, phosphoadenosine phosphosulfate reductase , phosphoribosylformyl glycinamidine synthase subunit, protein kinase, protein phosphatase, purine nucleoside phosphorylase, recombinase A, sll1032-protein, sodium/proton antiporter, ThiF family protein, threonine dehydratase, threonine efflux protein, transcription factor, or ymr013c-protein, which respectively encode a protein comprising a polypeptide encoded by a nucleic acid sequence as shown in Table I, application no. 11, column 5 or 8, (preferably the coding region thereof), or a homolog or a fragment thereof, which respectively encode a protein comprising a polypeptide as depicted in Table II, application no. 11, column 5 or 8, or a homolg or a fragment thereof, and/or which respectively can be amplified with the primer set shown in Table III, application no. 11, column 8, are also referred to as “FCRP genes”.

Proteins or polypeptides encoded by “FCRP-genes” are referred to as “Fine Chemical Related Proteins” or “FCRP”. For the purposes of the description of the present invention, the respective protein having an activity of 2-C-methyl-D-erythritol 4-phosphate cytidylyltransferase, 3-ketoacyl-CoA thiolase, 49747384_SOYBEAN-protein, AAS bifunctional protein, ABC metal ion transporter substrate-binding protein, acid phosphatase precursor, acid shock protein, aconitate hydratase, acyl transferase, arginine decarboxylase, aromatic acid decarboxylase, At1g19800-protein, At1g23110-protein, At2g45420-protein, ATPase epsilon subunit, b0050-protein, b1234-protein, b1248-protein, b1330-protein, b1670-protein, b1837-protein, b2032-protein, b2099-protein, b2107-protein, b2399-protein, b2682-protein, b2849-protein, b3814-protein, bifunctional protein (phosphoribosyltransferase and regulatory protein), branched chain amino acid ABC transporter ATP-binding protein, calcium-dependent protein kinase, carbamoyl-phosphate synthase subunit, carbon dioxide concentrating mechanism protein, cation-transporting ATPase, CCAAT-binding transcription factor, chlorophyllase, colanic acid biosynthesis protein, coproporphyrinogen III oxidase, DnaJ-like chaperone, ethanolamine utilization protein, F-box protein, Fe—S subunit of oxidoreductase, flavodoxin, glucose-6-phosphate 1-dehydrogenase, glutaredoxin, glutathione S-transferase, glycerol-3-phosphate dehydrogenase, heat shock transcription factor, HesB/YadR/YfhF family protein, homocitrate synthase, lipoprotein precursor, metal-dependent hydrolase, monothiol glutaredoxin, NADH-quinone oxidoreductase subunit, nitrate/nitrite transport protein, oxidoreductase, peptidyl-prolyl cis-trans isomerase, permease protein of ABC transporter, phosphate import ATP-binding protein, phosphoadenosine phosphosulfate reductase , phosphoribosylformyl glycinamidine synthase subunit, protein kinase, protein phosphatase, purine nucleoside phosphorylase, recombinase A, sll1032-protein, sodium/proton antiporter, ThiF family protein, threonine dehydratase, threonine efflux protein, transcription factor, or ymr013c-protein, the respective protein comprising a polypeptide encoded by one or more respective nucleic acid sequences as shown in Table 1, application no. 11, column 5 or 8, (preferably the coding region thereof), or a homolog or fragment thereof, the respective protein comprising a respective polypeptide as depicted in Table II, application no. 11, column 5 or 8, or a homolog or fragment thereof, the respective protein comprising a sequence corresponding to the consensus sequence as shown in Table IV, application no. 11, column 8, and/or the respective protein comprising at least one polypeptide motif as shown in Table IV, application no. 11, column 8 is also referred to as Fine Chemical Related Protein” or “FCRP”.

Thus, in one embodiment, the present invention provides a process of the production of palmitic acid, by increasing or generating one or more activities, especially selected from the group consisting of 2-C-methyl-D-erythritol 4-phosphate cytidylyltransferase, 3-ketoacyl-CoA thiolase, 49747384_SOYBEAN-protein, AAS bifunctional protein, ABC metal ion transporter substrate-binding protein, acid phosphatase precursor, acid shock protein, aconitate hydratase, acyl transferase, arginine decarboxylase, aromatic acid decarboxylase, At1g19800-protein, At1g23110-protein, At2g45420-protein, ATPase epsilon subunit, b0050-protein, b1234-protein, b1248-protein, b1330-protein, b1670-protein, b1837-protein, b2032-protein, b2099-protein, b2107-protein, b2399-protein, b2682-protein, b2849-protein, b3814-protein, bifunctional protein (phosphoribosyltransferase and regulatory protein), branched chain amino acid ABC transporter ATP-binding protein, calcium-dependent protein kinase, carbamoyl-phosphate synthase subunit, carbon dioxide concentrating mechanism protein, cation-transporting ATPase, CCAAT-binding transcription factor, chlorophyllase, colanic acid biosynthesis protein, coproporphyrinogen III oxidase, DnaJ-like chaperone, ethanolamine utilization protein, F-box protein, Fe—S subunit of oxidoreductase, flavodoxin, glucose-6-phosphate 1-dehydrogenase, glutaredoxin, glutathione S-transferase, glycerol-3-phosphate dehydrogenase, heat shock transcription factor, HesB/YadR/YfhF family protein, homocitrate synthase, lipoprotein precursor, metal-dependent hydrolase, monothiol glutaredoxin, NADH-quinone oxidoreductase subunit, nitrate/nitrite transport protein, oxidoreductase, peptidyl-prolyl cis-trans isomerase, permease protein of ABC transporter, phosphate import ATP-binding protein, phosphoadenosine phosphosulfate reductase, phosphoribosylformyl glycinamidine synthase subunit, protein kinase, protein phosphatase, purine nucleoside phosphorylase, recombinase A, sll1032-protein, sodium/proton antiporter, ThiF family protein, threonine dehydratase, threonine efflux protein, transcription factor, and ymr013c-protein, which is conferred by one or more FCRPs or the gene product of one or more FCRP-genes, for example by the gene product of a nucleic acid sequences comprising a polynucleotide selected from the group as shown in Table I, application no. 11, column 5 or 8, (preferably by the coding region thereof), or a homolog or a fragment thereof, e.g. or by one or more proteins each comprising a polypeptide encoded by one or more nucleic acid sequences selected from the group as shown in Table I, application no. 11, column 5 or 8, (preferably by the coding region thereof), or a homolog or a fragment thereof, or by one or more protein(s) each comprising a polypeptide selected from the group as depicted in Table II, application no. 11, column 5 and 8, or a homolog thereof, or a protein comprising a sequence corresponding to the consensus sequence or comprising at least one polypeptide motif as shown in Table IV, application no. 11, column 8.

for the disclosure of this paragraph see [0025.1.8.8] above.

In an embodiment, the process comprises increasing or generating the activity of one or more polypeptides having said activity, e.g. by generating or increasing the amount and/or specific activity in the cell or a compartment of a cell of one of more FCRP, especially selected from the group consisting of 2-C-methyl-D-erythritol 4-phosphate cytidylyltransferase, 3-ketoacyl-CoA thiolase, 49747384_SOYBEAN-protein, AAS bifunctional protein, ABC metal ion transporter substrate-binding protein, acid phosphatase precursor, acid shock protein, aconitate hydratase, acyl transferase, arginine decarboxylase, aromatic acid decarboxylase, At1g19800-protein, At1g23110-protein, At2g45420-protein, ATPase epsilon subunit, b0050-protein, b1234-protein, b1248-protein, b1330-protein, b1670-protein, b1837-protein, b2032-protein, b2099-protein, b2107-protein, b2399-protein, b2682-protein, b2849-protein, b3814-protein, bifunctional protein (phosphoribosyltransferase and regulatory protein), branched chain amino acid ABC transporter ATP-binding protein, calcium-dependent protein kinase, carbamoyl-phosphate synthase subunit, carbon dioxide concentrating mechanism protein, cation-transporting ATPase, CCAAT-binding transcription factor, chlorophyllase, colanic acid biosynthesis protein, coproporphyrinogen III oxidase, DnaJ-like chaperone, ethanolamine utilization protein, F-box protein, Fe—S subunit of oxidoreductase, flavodoxin, glucose-6-phosphate 1-dehydrogenase, glutaredoxin, glutathione S-transferase, glycerol-3-phosphate dehydrogenase, heat shock transcription factor, HesB/YadR/YfhF family protein, homocitrate synthase, lipoprotein precursor, metal-dependent hydrolase, monothiol glutaredoxin, NADH-quinone oxidoreductase subunit, nitrate/nitrite transport protein, oxidoreductase, peptidyl-prolyl cis-trans isomerase, permease protein of ABC transporter, phosphate import ATP-binding protein, phosphoadenosine phosphosulfate reductase , phosphoribosylformyl glycinamidine synthase subunit, protein kinase, protein phosphatase, purine nucleoside phosphorylase, recombinase A, sll1032-protein, sodium/proton antiporter, ThiF family protein, threonine dehydratase, threonine efflux protein, transcription factor, and ymr013c-protein, for example of the respective polypeptide as depicted in Table II, application no. 11, column 5 and 8, or a homolog or a fragment thereof, or the respective polypeptide comprising a sequence corresponding to the consensus sequences as shown in Table IV, application no. 11, column 8, or the respective polypeptide comprising at least one polypeptide motif as depicted in Table IV, application no. 11, column 8.

A further embodiment of the present invention relates to a process for the production of palmitic acid, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a chlorophyllase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a At1g19800-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a At1g23110-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a At2g45420-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a CCAAT-binding transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a ThiF family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a recombinase A non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a NADH-quinone oxidoreductase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a HesB/YadR/YfhF family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a peptidyl-prolyl cis-trans isomerase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a purine nucleoside phosphorylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a b0050-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a sodium/proton antiporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a b1234-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a b1248-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a b1330-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a lipoprotein precursor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a Fe—S subunit of oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a acid shock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a b1670-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a b1837-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a b2032-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a colanic acid biosynthesis protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a b2099-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a b2107-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a b2399-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a ethanolamine utilization protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a b2682-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a 2-C-methyl-D-erythritol 4-phosphate cytidylyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a flavodoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a AAS bifunctional protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a b2849-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a b3814-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a threonine efflux protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a 3-ketoacyl-CoA thiolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a bifunctional protein (phosphoribosyltransferase and regulatory protein) non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a branched chain amino acid ABC transporter

ATP-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a carbon dioxide concentrating mechanism protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a sll1032-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a phosphoribosylformyl glycinamidine synthase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a nitrate/nitrite transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a carbamoyl-phosphate synthase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a glutathione S-transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a ABC metal ion transporter substrate-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a acyl transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a coproporphyrinogen III oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a cation-transporting ATPase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a permease protein of ABC transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a aromatic acid decarboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a phosphate import ATP-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a arginine decarboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a phosphoadenosine phosphosulfate reductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a threonine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a metal-dependent hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a homocitrate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a acid phosphatase precursor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a glycerol-3-phosphate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a F-box protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a DnaJ-like chaperone non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a aconitate hydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a ymr013c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a ATPase epsilon subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a 49747384_SOYBEAN-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a glucose-6-phosphate 1-dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating the activity of a polypeptide comprising a polypeptide as depicted in the respective line in column 5 or 8 of Table II, application no. 11, or a homolog or a fragment thereof, a consensus sequence or at least one polypeptide motif as depicted in the respective line in column 8 of Table IV, application no. 11; or
increasing or generating the activity of an expression product of one or more nucleic acid molecule(s) comprising a polynucleotide as depicted in the respective line in column 5 or 8 of Table I, application no. 11, preferably the coding region thereof, or a homolog or a fragment thereof;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of palmitic acid, or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of chlorophyllase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of At1g19800-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of At1g23110-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of At2g45420-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of CCAAT-binding transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of ThiF family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of recombinase A in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of NADH-quinone oxidoreductase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of HesB/YadR/YfhF family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of peptidyl-prolyl cis-trans isomerase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of purine nucleoside phosphorylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of b0050-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of sodium/proton antiporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of b1234-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of b1248-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of b1330-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of lipoprotein precursor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of Fe—S subunit of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of acid shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of b1670-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of b1837-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of b2032-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of colanic acid biosynthesis protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of b2099-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of b2107-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of b2399-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of ethanolamine utilization protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of b2682-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of 2-C-methyl-D-erythritol 4-phosphate cytidylyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of flavodoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of AAS bifunctional protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of b2849-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of b3814-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of threonine efflux protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of 3-ketoacyl-CoA thiolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of bifunctional protein (phosphoribosyltransferase and regulatory protein) in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of branched chain amino acid ABC transporter ATP-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of carbon dioxide concentrating mechanism protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of sll1032-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of phosphoribosylformyl glycinamidine synthase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of nitrate/nitrite transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of carbamoyl-phosphate synthase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of glutathione S-transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of ABC metal ion transporter substrate-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of acyl transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of coproporphyrinogen III oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of cation-transporting ATPase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of permease protein of ABC transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of aromatic acid decarboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of phosphate import ATP-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of arginine decarboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of phosphoadenosine phosphosulfate reductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of threonine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of metal-dependent hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of homocitrate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of acid phosphatase precursor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of glycerol-3-phosphate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of F-box protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of DnaJ-like chaperone in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of aconitate hydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of ymr013c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of ATPase epsilon subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of 49747384_SOYBEAN-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a) increasing or generating one or more activities selected from the group consisting of glucose-6-phosphate 1-dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

A further embodiment of the present invention relates to a process for the production of palmitic acid, which comprises
(a1) increasing or generating the activity of a polypeptide comprising a polypeptide as depicted in the respective line in column 5 or 8 of Table II, application no. 11, or a homolog or fragment thereof, a consensus sequence or at least one polypeptide motif as depicted in column 8 of Table IV, application no. 11; or
increasing or generating the activity of an expression product of one or more nucleic acid molecule(s) comprising a polynucleotide as depicted in the respective line in column 5 or 8 of Table I, application no. 11, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 11, or a homolog or a fragment thereof, a consensus sequence or at least one polypeptide motif as depicted in the respective line in column 8 of Table IV, application no. 11, which is joined to a transit peptide; or
increasing or generating the activity of an expression product of one or more nucleic acid molecule(s) comprising a polynucleotide as depicted in the respective line in column 5 or 8 of Table I, application no. 11, preferably the coding region thereof, or a homolog or a fragment thereof, which is joined to a nucleic acid sequence encoding an organelle localization sequence, preferably a plastid or a mitochondrion localization sequence, especially a plastid localization sequence;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 11, or a homolog or a fragment thereof, a consensus sequence or at least one polypeptide motif as depicted in the respective line in column 8 of Table IV, application no. 11; or increasing or generating the activity of an expression product of one or more nucleic acid molecule(s) comprising a polynucleotide as depicted in the respective line in column 5 or 8 of Table I, application no. 11, preferably the coding region thereof, or a homolog or a fragment thereof;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of palmitic acid, or a composition comprising palmitic acid in said non-human organism or in the culture medium surrounding said non-human organism.

Accordingly, the present invention relates to a process for the production of palmitic acid, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a chlorophyllase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a At1g19800-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a At1g23110-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a At2g45420-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a CCAAT-binding transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a ThiF family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a recombinase A in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a NADH-quinone oxidoreductase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a HesB/YadR/YfhF family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a peptidyl-prolyl cis-trans isomerase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a purine nucleoside phosphorylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a b0050-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a sodium/proton antiporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a b1234-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a b1248-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a b1330-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a lipoprotein precursor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a Fe—S subunit of oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a acid shock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a b1670-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a b1837-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a b2032-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a colanic acid biosynthesis protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a b2099-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a b2107-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a b2399-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a ethanolamine utilization protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a b2682-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a 2-C-methyl-D-erythritol 4-phosphate cytidylyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a flavodoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a AAS bifunctional protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a b2849-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a b3814-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a threonine efflux protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a 3-ketoacyl-CoA thiolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a bifunctional protein (phosphoribosyltransferase and regulatory protein) in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a branched chain amino acid ABC transporter

ATP-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a carbon dioxide concentrating mechanism protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a sll1032-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a phosphoribosylformyl glycinamidine synthase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a nitrate/nitrite transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a carbamoyl-phosphate synthase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a glutathione S-transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a ABC metal ion transporter substrate-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a acyl transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a coproporphyrinogen III oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a cation-transporting ATPase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a permease protein of ABC transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a aromatic acid decarboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a phosphate import ATP-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a arginine decarboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a phosphoadenosine phosphosulfate reductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a threonine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a metal-dependent hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a homocitrate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a acid phosphatase precursor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a glycerol-3-phosphate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a F-box protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a DnaJ-like chaperone in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a aconitate hydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a ymr013c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a ATPase epsilon subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a 49747384_SOYBEAN-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a glucose-6-phosphate 1-dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Accordingly, the present invention relates to a process for the production of palmitic acid, which comprises
(a) increasing or generating the activity of a polypeptide comprising a polypeptide as depicted in the respective line in column 5 or 8 of Table II, application no. 11, or a homolog or a fragment thereof, a consensus sequence or at least one polypeptide motif as depicted in the respective line in column 8 of Table IV, application no. 11; or
increasing or generating the activity of an expression product of one or more nucleic acid molecule(s) comprising a polynucleotide as depicted in the respective line in column 5 or 8 of Table I, application no. 11, preferably the coding region thereof, or a homolog or a fragment thereof;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of palmitic acid, or a composition comprising palmitic acid in said non-human organism or in the culture medium surrounding said non-human organism.

In a further embodiment the activity of the polypeptide comprising a polypeptide as depicted in the respective line in column 5 or 8 of Table II, application no. 11, or a homolog or a fragment thereof, a consensus sequence or at least one polypeptide motif as depicted in the respective line in column 8 of Table IV, application no. 11, is increased or generated non-targeted in the above-mentioned process in a microorganism or plant or a part thereof.

In a further embodiment said polypeptide has the activity of the respective polypeptide represented by a protein comprising a polypeptide as depicted in the respective line in column 5 of Table II, application no. 11.

In a further embodiment the activity of the expression product of one or more nucleic acid molecule(s) comprising a polynucleotide as depicted in the respective line in column 5 or 8 of Table I, application no. 11, preferably the coding region thereof, or a homolog or a fragment thereof, is increased or generated non-targeted in the above-mentioned process in a microorganism or plant or a part thereof.

In a further embodiment the activity of the polypeptide comprising a polypeptide as depicted in the respective line in column 5 or 8 of Table II, application no. 11, or a homolog or a fragment thereof, a consensus sequence or at least one polypeptide motif as depicted in the respective line in column 8 of Table IV, application no. 11, is increased or generated in the above-mentioned process in an organelle preferably in plastids or mitochondria, especially in plastids, of a microorganism or plant.

In a further embodiment said polypeptide has the activity of the respective polypeptide represented by a protein comprising a polypeptide as depicted in the respective line in column 5 of Table II, application no. 11.

In a further embodiment the activity of the expression product of one or more nucleic acid molecule(s) comprising a polynucleotide as depicted in the respective line in column 5 or 8 of Table I, application no. 11, preferably the coding region thereof, or a homolog or a fragment thereof, is increased or generated in the above-mentioned process in an organelle, preferably in plasids or mitochondria, especially in plastids, of a microorganism or plant.

In a further embodiment the activity of the polypeptide comprising a polypeptide as depicted in the respective line in column 5 or 8 of Table II, application no. 11, or a homolog or a fragment thereof, a consensus sequence or at least one polypeptide motif as depicted in the respective line in column 8 of Table IV, application no. 11, is increased or generated in the above-mentioned process in the cyctosol of a cell, of a microorganism or plant.

In a further embodiment said polypeptide has the activity of the respective polypeptide represented by a protein comprising a polypeptide as depicted in the respective line in column 5 of Table II, application no. 11.

In a further embodiment the activity of the expression product of one or more nucleic acid molecule(s) comprising a polynucleotide as depicted in the respective line in column 5 or 8 of Table I, application no. 11, preferably the coding region thereof, or a homolog or a fragment thereof, is increased or generated in the above-mentioned process in the cytosol of a cell, of a microorganism or plant.

[0039.1.8.11] to [0066.1.8.11] for the disclosure of these paragraphs see [0039.1.8.8] to [0066.1.8.8] above.

As mentioned above the nucleic acid sequences coding for the proteins as shown in Table II, application no. 11, column 5 or 8, or homologs or fragments thereof, are joined to a nucleic acid sequence encoding a plastidic transit peptide, e.g, if for the nucleic acid molecule in Table I, column 6 the term “plastidic” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in Table II, application no. 11, column 5 or 8, or homologs or fragments thereof, e.g., if for the nucleic acid molecule in Table I, column 6 the term “plastidic” is indicated.

[0068.1.8.11] to [0072.1.8.11] for the disclosure of these paragraphs see [0068.1.8.8] to [0072.1.8.8] above.

As mentioned above the nucleic acid sequences coding for the proteins as shown in the respective line in Table II, application no. 11, column 5 or 8, or homologs or fragments thereof, are joined to a nucleic acid sequence encoding a mitochondric transit peptide, e.g., if for the respective nucleic acid molecule in Table I, column 6 the term “mitochondrial” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in the respective line in Table II, application no. 11, column 5 or 8, or homologs or fragments thereof, e.g., if for the nucleic acid molecule in the respective line in Table I, column 6 the term “mitochondrial” is indicated. (For th eavoidance ofdoubt, th eterm “mitochondric” and “mitochondrial” are interchangeable.)

[0074.1.8.11] to [0075.1.8.11] for the disclosure of these paragraphs see [0074.1.8.8] to [0075.1.8.8] above.

Nucleic acid sequences coding for the transit peptides may be chemically synthesized either in part or wholly according to structure of transit peptide sequences disclosed in the prior art. Said natural or chemically synthesized sequences can be directly linked to the sequences encoding the mature protein or via a linker nucleic acid sequence, which may be typically less than 500 base pairs, preferably less than 450, 400, 350, 300, 250 or 200 base pairs, more preferably less than 150, 100, 90, 80, 70, 60, 50, 40 or 30 base pairs and most preferably less than 25, 20, 15, 12, 9, 6 or 3 base pairs in length and are in frame to the coding sequence. Furthermore favorable nucleic acid sequences encoding transit peptides may comprise sequences derived from more than one biological and/or chemical source and may include a nucleic acid sequence derived from the amino-terminal region of the mature protein, which in its native state is linked to the transit peptide. In a preferred embodiment of the invention said amino-terminal region of the mature protein is typically less than 150 amino acids, preferably less than 140, 130, 120, 110, 100 or 90 amino acids, more preferably less than 80, 70, 60, 50, 40, 35, 30, 25 or 20 amino acids and most preferably less than 19, 18, 17, 16, 15, 14, 13, 12, 11 or 10 amino acids in length. But even shorter or longer stretches are also possible. In addition target sequences, which facilitate the transport of proteins to other cell compartments such as the vacuole, endoplasmic reticulum, golgi complex, glyoxysomes, peroxisomes, mitochondria (in case of a plastidal transit peptide) or plastids, especially chloroplasts (in case of a mitochondric transit peptide) may be also part of the inventive nucleic acid sequence. The proteins translated from said inventive nucleic acid sequences are a kind of fusion proteins that means the nucleic acid sequences encoding the plastidal transit peptide for example the ones shown in Table a, preferably the last one of the Table a are joint to the respective nucleic acid sequences shown in Table I, application no. 11, columns 5 or 8, especially the coding region thereof, or homologs or fragments thereof, in case of nucleic acid sequences being expressed in plastids, and that means the nucleic acid sequences encoding the mitochondric transit peptide for example the ones shown in Table b, preferably the last one of the Table b are joint to the respective nucleic acid sequences shown in Table I, application no. 11, columns 5 or 8, especially the coding region thereof, or homologs or fragments thereof, in case of nucleic acid sequences being expressed in mitochondria. The person skilled in the art is able to join said sequences in a functional manner. Advantageously the transit peptide part is cleaved off from the protein part shown in Table II, application no. 11, columns 5 or 8, or a homolog or fragment thereof, during or after the transport into the organelles, preferably into the plastids or mitochondria, respectively.

All products of the cleavage of the preferred transit peptide shown in the last line of Table a have preferably the N-terminal amino acid sequences QIA CSS or QIA EFQLTT in front of the start methionine of the respective protein mentioned in Table II, application no. 11, column 5 or 8. or homologs or fragments thereof. Other short amino acid sequences of an range of 1 to 20 amino acids preferable 2 to 15 amino acids, more preferable 3 to 10 amino acids most preferably 4 to 8 amino acids are also possible in front of the start methionine of the protein mentioned in Table II, application no. 11, columns 5 or 8, or homologs or fragments thereof. In case of the amino acid sequence QIA CSS the three amino acids in front of the start methionine are stemming from the LIC (=ligatation independent cloning) cassette. Said short amino acid sequence is preferred in the case of the expression of E. coli genes. In case of the amino acid sequence QIA EFQLTT the six amino acids in front of the start methionine are stemming from the LIC cassette. Said short amino acid sequence is preferred in the case of the expression of S. cerevisiae genes. The skilled worker knowns that other short sequences are also useful in the expression of the respective nucleic acid molecule comprising a polynucleotide as depicted in Table I, application no. 11, column 5 or 8, preferably the coding region thereof, or a homolog or a fragment thereof. Furthermore the skilled worker is aware of the fact that there is not a need for such short sequences in the expression of the genes.

[0077.1.8.11] to [0078.1.8.11] for the disclosure of these paragraphs see [0077.1.8.8] to [0078.1.8.8] above.

Alternatively to the targeting of the respective sequences shown in Table II, application no. 11, columns 5 or 8, or homologs or fragments thereof, preferably of sequences in general encoded in the nucleus with the aid of the targeting sequences mentioned for example in Table a alone, Table b alone, or in combination with other targeting sequences preferably into plastids or mitochondria, respectively, the nucleic acids of the invention can directly be introduced into the plastidal or mitochondrial genome. Therefore in a preferred embodiment the respective nucleic acid sequences shown in the respective line in Table I, application no. 11, columns 5 or 8, preferably the coding region thereof, or homologs or fragments thereof, are directly introduced and expressed in plastids e.g., if for the nucleic acid molecule in the respective line in Table I, column 6, the term “plastidic” is indicated, or are directly introduced and expressed in mitochondria e.g., if for the nucleic acid molecule in the respective line in Table I, column 6 the term “mitochondrial” is indicated.

The term “introduced” in the context of this specification shall mean the insertion of a nucleic acid sequence into the organism by means of a “transfection”, “transduction” or preferably by “transformation”.

A plastid, such as a chloroplast, or a mitochondrion, has been “transformed” by an exogenous (preferably foreign) nucleic acid sequence if a nucleic acid sequence has been introduced into the plastid or mitochondrion that means that this sequence has crossed the membrane or the membranes of the plastid or of the mitochondrion. The exogenous, preferably foreign, DNA may be integrated (covalently linked) into plastid or mitochondrial DNA making up the genome of the plastid or mitochondrion, or it may remain unintegrated (e.g., by including a chloroplast origin of replication). “Stably” integrated DNA sequences are those, which are inherited through plastid or mitochondrion replication, thereby transferring new plastids or mitochondria, with the features of the integrated DNA sequence to the progeny.

[0080.1.8.11] to [0083.1.8.11] for the disclosure of these paragraphs see [0080.1.8.8] to 0083.1.8.8] above.

For the process of the present invention it may be of great advantage that by transforming the plastids the intraspecies specific transgene flow is blocked, because a lot of species such as corn, cotton and rice have a strict maternal inheritance of plastids. By placing the respective genes specified in the respective line in Table I, application no. 11, columns 5 or 8, preferably the coding region, or homologs or fragments thereof, e.g. if for the nucleic acid molecule in the respective line in column 6 of Table I the term “plastidic” is indicated, in the plastids of plants, these genes will not be present in the pollen of said plants.

A further preferred embodiment of the invention relates to the use of so called “chloroplast localization sequences”, in which a first RNA sequence or molecule is capable of transporting or “chaperoning” a second RNA sequence, such as a RNA sequence transcribed from the sequences depicted in the respective line in Table I, application no. 11, columns 5 or 8, preferably the coding region thereof, or a homolog or a fragment thereof, or a sequence encoding a protein, as depicted in the respective line in Table II, application no. 11, columns 5 and 8, or a homolog or a fragment thereof, from an external environment inside a cell or outside a plastid into a chloroplast. In one embodiment the chloroplast localization signal is substantially similar or complementary to a complete or intact viroid sequence. The chloroplast localization signal may be encoded by a DNA sequence, which is transcribed into the chloroplast localization RNA. The term “viroid” refers to a naturally occurring single stranded RNA molecule (Flores, C. R. Acad. Sci. III. 324 (10), 943 (2001)). Viroids usually contain about 200-500 nucleotides and generally exist as circular molecules. Examples of viroids that contain chloroplast localization signals include but are not limited to ASBVd, PLMVd, CChMVd and ELVd. The viroid sequence or a functional part of it can be fused to the sequences depicted in the respective line in Table I, application no. 11, columns 5 or 8, preferably the coding region thereof, or a homolog or a fragment thereof, or a sequence encoding a protein, as depicted in the respective line in Table II, application no. 11, columns 5 or 8, or a homolog or a fragment thereof, in such a manner that the viroid sequence transports a sequence transcribed from a sequence as depicted in the respective line in Table I, application no. 11 columns 5 or 8, preferably the coding region thereof, or a homolog or a fragment thereof, or a sequence encoding a protein as depicted in the respective line in Table II, application no. 11 columns 5 or 8, or a homolog or a fragment thereof, into the chloroplasts, e.g. if for the nucleic acid molecule in column 6 of Table I the term “plastidic” is indicated. A preferred embodiment uses a modified ASBVd (Navarro et al., Virology. 268 (1), 218 (2000)).

In a further specific embodiment the protein to be expressed in the plastids such as the proteins depicted in the respective line in Table II, application no. 11, columns 5 or 8, or a homolog or a fragment thereof, e.g. if for the nucleic acid molecule in the respective line in column 6 of Table I the term “plastidic” is indicated, are encoded by different nucleic acids. Such a method is disclosed in WO 2004/040973, which shall be incorporated by reference. WO 2004/040973 teaches a method, which relates to the translocation of an RNA corresponding to a gene or gene fragment into the chloroplast by means of a chloroplast localization sequence. The genes, which should be expressed in the plant or plant cells, are split into nucleic acid fragments, which are introduced into different compartments in the plant e.g. the nucleus, the plastids and/or mitochondria. Additionally plant cells are described in which the chloroplast contains a ribozyme fused at one end to an RNA encoding a fragment of a protein used in the inventive process such that the ribozyme can trans-splice the translocated fusion RNA to the RNA encoding the gene fragment to form and as the case may be reunite the nucleic acid fragments to an intact mRNA encoding a functional protein for example as disclosed in the respective line in Table II, application no. 11, columns 5 or 8, or a homolog or a fragment thereof.

In a preferred embodiment of the invention the nucleic acid sequences as shown in the respective line in Table I, application no. 11, columns 5 or 8, preferably the coding region thereof, or homologs or fragments thereof, e.g. if for the nucleic acid molecule in the respective line in column 6 of Table I the term “plastidic” is indicated, used in the inventive process are transformed into plastids, which are metabolically active. Those plastids should preferably maintain at a high copy number in the plant or plant tissue of interest, most preferably the chloroplasts found in green plant tissues, such as leaves or cotyledons or in seeds.

For a good expression in the plastids the nucleic acid sequences as shown in the respective line in Table I, application no. 11, columns 5 or 8, preferably the coding region thereof, or homologs or fragments thereof, e.g. if for the nucleic acid molecule in the respective line in column 6 of Table I the term “plastidic” is indicated, are introduced into an expression cassette using preferably a promoter and terminater, which are active in plastids preferably a chloroplast promoter. Examples of such promoters include the psbA promoter from the gene from spinach or pea, the rbcL promoter, and the atpB promoter from corn.

[0089.1.8.11] to [0092.1.8.11] for the disclosure of these paragraphs see [0089.1.8.8] to [0092.1.8.8] above.

Advantageously the process for the production of the fine chemical palmitic acid leads to an enhanced production of the respective fine chemical. The terms “enhanced” or “increase” mean at least a 10%, 20%, 30%, 40% or 50%, preferably at least 60%, 70%, 80%, 90% or 100%, more preferably 150%, 200%, 300%, 400% or 500% higher production of the respective fine chemical palmitic acid in comparison to the wild-type as defined above, e.g. that means in comparison to a non-human organism without the aforementioned modification of the activity of a protein as shown in the respective line in Table II, application no. 11, column 5 or 8, or a fragment or a homolog thereof. The modification of the activity of a protein as shown in the respective line in Table II, application no. 11, column 5 or 8, or a homolog or a fragment thereof, or their combination can be achieved by joining the protein to a respective transit peptide, e.g. if for the respective encoding nucleic acid molecule in column 6 of Table I the term “plastidic” or “mitochondrial” is indicated.

Surprisingly it was found, that the transgenic expression of a protein as shown in the respective line in Table II, application no. 11, column 5, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, conferred a production of or an increase in palmitic acid,respectively, to the transgenic non-human organism as compared to a corresponding non-transformed wild type.

In one embodiment the transgenic expression of a protein as shown in the respective line in Table II, application no. 11, column 5 or 8, or a homolog or a fragment thereof, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, confers a production of or an increase in palmitic acid,respectively, to the transgenic non-human organism as compared to a corresponding non-transformed wild type.

Accordingly, in one embodiment, an increase of palmitic acid in a non-human organism, as compared to a corresponding non-transformed wild type non-human organism, is conferred in the process of the invention, if the activity of a polypeptide showing the activity of a protein phosphatase, or if the activity of the polypeptide At1g07430, preferably represented by SEQ ID NO. 17452, or a homolog or fragment thereof, or if the activity of a polypeptide encoded by a nucleic acid molecule comprising the nucleic acid SEQ ID NO. 17451, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 17451 or polypeptide SEQ ID NO. 17452, respectively, or a homolog or a fragment thereof, is increased or generated, or if the activity protein phosphatase is increased or generated in a non-human organism, like a microorganism or a plant cell, plant or part thereof, especially with non-targeted localization. For example, an increase of the palmitic acid of at least 1 percent, particularly in a range of 16 to 36-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a chlorophyllase, or if the activity of the polypeptide At1g19670, preferably represented by SEQ ID NO. 113652, or a homolog or fragment thereof, or if the activity of a polypeptide encoded by a nucleic acid molecule comprising the nucleic acid SEQ ID NO. 113651, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 113651 or polypeptide SEQ ID NO. 113652, respectively, or a homolog or a fragment thereof, is increased or generated, or if the activity chlorophyllase is increased or generated in a non-human organism, like a microorganism or a plant cell, plant or part thereof, especially with non-targeted localization. For example, an increase of the palmitic acid of at least 1 percent, particularly in a range of 15 to 27-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a At1g19800-protein, or if the activity of the polypeptide At1g19800, preferably represented by SEQ ID NO. 17638, or a homolog or fragment thereof, or if the activity of a polypeptide encoded by a nucleic acid molecule comprising the nucleic acid SEQ ID NO. 17637, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 17637 or polypeptide SEQ ID NO. 17638, respectively, or a homolog or a fragment thereof, is increased or generated, or if the activity At1g19800-protein is increased or generated in a non-human organism, like a microorganism or a plant cell, plant or part thereof, especially with non-targeted localization. For example, an increase of the palmitic acid of at least 1 percent, particularly in a range of 18 to 29-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a At1g23110-protein, or if the activity of the polypeptide At1g23110, preferably represented by SEQ ID NO. 102087, or a homolog or fragment thereof, or if the activity of a polypeptide encoded by a nucleic acid molecule comprising the nucleic acid SEQ ID NO. 102086, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 102086 or polypeptide SEQ ID NO. 102087, respectively, or a homolog or a fragment thereof, is increased or generated, or if the activity At1g23110-protein is increased or generated in a non-human organism, like a microorganism or a plant cell, plant or part thereof, especially with non-targeted localization. For example, an increase of the palmitic acid of at least 1 percent, particularly in a range of 24 to 58-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a transcription factor, or if the activity of the polypeptide At1g68320, preferably represented by SEQ ID NO. 1062, or a homolog or fragment thereof, or if the activity of a polypeptide encoded by a nucleic acid molecule comprising the nucleic acid SEQ ID NO. 1061, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 1061 or polypeptide SEQ ID NO. 1062, respectively, or a homolog or a fragment thereof, is increased or generated, or if the activity transcription factor is increased or generated in a non-human organism, like a microorganism or a plant cell, plant or part thereof, especially with non-targeted localization. For example, an increase of the palmitic acid of at least 1 percent, particularly in a range of 16 to 84-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a protein phosphatase, or if the activity of the polypeptide At1g72770, preferably represented by SEQ ID NO. 19420, or a homolog or fragment thereof, or if the activity of a polypeptide encoded by a nucleic acid molecule comprising the nucleic acid SEQ ID NO. 19419, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 19419 or polypeptide SEQ ID NO. 19420, respectively, or a homolog or a fragment thereof, is increased or generated, or if the activity protein phosphatase is increased or generated in a non-human organism, like a microorganism or a plant cell, plant or part thereof, especially with non-targeted localization. For example, an increase of the palmitic acid of at least 1 percent, particularly in a range of 16 to 54-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a At2g45420-protein, or if the activity of the polypeptide At2g45420, preferably represented by SEQ ID NO. 1624, or a homolog or fragment thereof, or if the activity of a polypeptide encoded by a nucleic acid molecule comprising the nucleic acid SEQ ID NO. 1623, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 1623 or polypeptide SEQ ID NO. 1624, respectively, or a homolog or a fragment thereof, is increased or generated, or if the activity At2g45420-protein is increased or generated in a non-human organism, like a microorganism or a plant cell, plant or part thereof, especially with non-targeted localization. For example, an increase of the palmitic acid of at least 1 percent, particularly in a range of 18 to 48-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a heat shock transcription factor, or if the activity of the polypeptide At3g02990, preferably represented by SEQ ID NO. 68658, or a homolog or fragment thereof, or if the activity of a polypeptide encoded by a nucleic acid molecule comprising the nucleic acid SEQ ID NO. 68657, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 68657 or polypeptide SEQ ID NO. 68658, respectively, or a homolog or a fragment thereof, is increased or generated, or if the activity heat shock transcription factor is increased or generated in a non-human organism, like a microorganism or a plant cell, plant or part thereof, especially with non-targeted localization. For example, an increase of the palmitic acid of at least 1 percent, particularly in a range of 15 to 33-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a CCAAT-binding transcription factor, or if the activity of the polypeptide At3g20910, preferably represented by SEQ ID NO. 22922, or a homolog or fragment thereof, or if the activity of a polypeptide encoded by a nucleic acid molecule comprising the nucleic acid SEQ ID NO. 22921, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 22921 or polypeptide SEQ ID NO. 22922, respectively, or a homolog or a fragment thereof, is increased or generated, or if the activity CCAAT-binding transcription factor is increased or generated in a non-human organism, like a microorganism or a plant cell, plant or part thereof, especially with non-targeted localization. For example, an increase of the palmitic acid of at least 1 percent, particularly in a range of 19 to 41-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization. For example, an increase of the palmitic acid of at least 1 percent, particularly in a range of 19 to 40 percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a monothiol glutaredoxin, or if the activity of the polypeptide At4g15670, preferably represented by SEQ ID NO. 3280, or a homolog or fragment thereof, or if the activity of a polypeptide encoded by a nucleic acid molecule comprising the nucleic acid SEQ ID NO. 3279, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 3279 or polypeptide SEQ ID NO. 3280, respectively, or a homolog or a fragment thereof, is increased or generated, or if the activity monothiol glutaredoxin is increased or generated in a non-human organism, like a microorganism or a plant cell, plant or part thereof, especially with non-targeted localization. For example, an increase of the palmitic acid of at least 1 percent, particularly in a range of 16 to 75-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a heat shock transcription factor, or if the activity of the polypeptide At4g18880, preferably represented by SEQ ID NO. 24233, or a homolog or fragment thereof, or if the activity of a polypeptide encoded by a nucleic acid molecule comprising the nucleic acid SEQ ID NO. 24232, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 24232 or polypeptide SEQ ID NO. 24233, respectively, or a homolog or a fragment thereof, is increased or generated, or if the activity heat shock transcription factor is increased or generated in a non-human organism, like a microorganism or a plant cell, plant or part thereof, especially with non-targeted localization. For example, an increase of the palmitic acid of at least 1 percent, particularly in a range of 19 to 23-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a protein phosphatase, or if the activity of the polypeptide At4g26080, preferably represented by SEQ ID NO. 68850, or a homolog or fragment thereof, or if the activity of a polypeptide encoded by a nucleic acid molecule comprising the nucleic acid SEQ ID NO. 68849, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 68849 or polypeptide SEQ ID NO. 68850, respectively, or a homolog or a fragment thereof, is increased or generated, or if the activity protein phosphatase is increased or generated in a non-human organism, like a microorganism or a plant cell, plant or part thereof, especially with non-targeted localization. For example, an increase of the palmitic acid of at least 1 percent, particularly in a range of 16 to 18-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a glutaredoxin, or if the activity of the polypeptide At4g33040, preferably represented by SEQ ID NO. 4103, or a homolog or fragment thereof, or if the activity of a polypeptide encoded by a nucleic acid molecule comprising the nucleic acid SEQ ID NO. 4102, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 4102 or polypeptide SEQ ID NO. 4103, respectively, or a homolog or a fragment thereof, is increased or generated, or if the activity glutaredoxin is increased or generated in a non-human organism, like a microorganism or a plant cell, plant or part thereof, especially with non-targeted localization. For example, an increase of the palmitic acid of at least 1 percent, particularly in a range of 19 to 36-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization. For example, an increase of the palmitic acid of at least 1 percent, particularly in a range of 16 to 40-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a protein phosphatase, or if the activity of the polypeptide At5g57050, preferably represented by SEQ ID NO. 5319, or a homolog or fragment thereof, or if the activity of a polypeptide encoded by a nucleic acid molecule comprising the nucleic acid SEQ ID NO. 5318, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 5318 or polypeptide SEQ ID NO. 5319, respectively, or a homolog or a fragment thereof, is increased or generated, or if the activity protein phosphatase is increased or generated in a non-human organism, like a microorganism or a plant cell, plant or part thereof, especially with non-targeted localization. For example, an increase of the palmitic acid of at least 1 percent, particularly in a range of 17 to 43-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a protein phosphatase, or if the activity of the polypeptide At5g59220, preferably represented by SEQ ID NO. 25499, or a homolog or fragment thereof, or if the activity of a polypeptide encoded by a nucleic acid molecule comprising the nucleic acid SEQ ID NO. 25498, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 25498 or polypeptide SEQ ID NO. 25499, respectively, or a homolog or a fragment thereof, is increased or generated, or if the activity protein phosphatase is increased or generated in a non-human organism, like a microorganism or a plant cell, plant or part thereof, especially with non-targeted localization. For example, an increase of the palmitic acid of at least 1 percent, particularly in a range of 19 to 400-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a oxidoreductase, or if the activity of the polypeptide AvinDRAFT_{—}1788, preferably represented by SEQ ID NO. 114084, or a homolog or fragment thereof, or if the activity of a polypeptide encoded by a nucleic acid molecule comprising the nucleic acid SEQ ID NO. 114083, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 114083 or polypeptide SEQ ID NO. 114084, respectively, or a homolog or a fragment thereof, is increased or generated, or if the activity oxidoreductase is increased or generated in a non-human organism, like a microorganism or a plant cell, plant or part thereof, especially with non-targeted localization. For example, an increase of the palmitic acid of at least 1 percent, particularly in a range of 17 to 33-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a ThiF family protein, or if the activity of the polypeptide AvinDRAFT_{—}3577, preferably represented by SEQ ID NO. 111156, or a homolog or fragment thereof, or if the activity of a polypeptide encoded by a nucleic acid molecule comprising the nucleic acid SEQ ID NO. 111155, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 111155 or polypeptide SEQ ID NO. 111156, respectively, or a homolog or a fragment thereof, is increased or generated, or if the activity ThiF family protein is increased or generated in a non-human organism, like a microorganism or a plant cell, plant or part thereof, especially with non-targeted localization. For example, an increase of the palmitic acid of at least 1 percent, particularly in a range of 18 to 49-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a recombinase A, or if the activity of the polypeptide AvinDRAFT_{—}3629, preferably represented by SEQ ID NO. 114232, or a homolog or fragment thereof, or if the activity of a polypeptide encoded by a nucleic acid molecule comprising the nucleic acid SEQ ID NO. 114231, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 114231 or polypeptide SEQ ID NO. 114232, respectively, or a homolog or a fragment thereof, is increased or generated, or if the activity recombinase A is increased or generated in a non-human organism, like a microorganism or a plant cell, plant or part thereof, especially with non-targeted localization. For example, an increase of the palmitic acid of at least 1 percent, particularly in a range of 16 to 51-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a NADH-quinone oxidoreductase subunit, or if the activity of the polypeptide AvinDRAFT_{—}4606, preferably represented by SEQ ID NO. 73720, or a homolog or fragment thereof, or if the activity of a polypeptide encoded by a nucleic acid molecule comprising the nucleic acid SEQ ID NO. 73719, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 73719 or polypeptide SEQ ID NO. 73720, respectively, or a homolog or a fragment thereof, is increased or generated, or if the activity NADH-quinone oxidoreductase subunit is increased or generated in a non-human organism, like a microorganism or a plant cell, plant or part thereof, especially with non-targeted localization. For example, an increase of the palmitic acid of at least 1 percent, particularly in a range of 18 to 57-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a HesB/YadR/YfhF family protein, or if the activity of the polypeptide AvinDRAFT_{—}5467, preferably represented by SEQ ID NO. 32649, or a homolog or fragment thereof, or if the activity of a polypeptide encoded by a nucleic acid molecule comprising the nucleic acid SEQ ID NO. 32648, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 32648 or polypeptide SEQ ID NO. 32649, respectively, or a homolog or a fragment thereof, is increased or generated, or if the activity HesB/YadR/YfhF family protein is increased or generated in a non-human organism, like a microorganism or a plant cell, plant or part thereof, especially with non-targeted localization. For example, an increase of the palmitic acid of at least 1 percent, particularly in a range of 17 to 61-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a peptidyl-prolyl cis-trans isomerase, or if the activity of the polypeptide AvinDRAFT_{—}6075, preferably represented by SEQ ID NO. 6675, or a homolog or fragment thereof, or if the activity of a polypeptide encoded by a nucleic acid molecule comprising the nucleic acid SEQ ID NO. 6674, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 6674 or polypeptide SEQ ID NO. 6675, respectively, or a homolog or a fragment thereof, is increased or generated, or if the activity peptidyl-prolyl cis-trans isomerase is increased or generated in a non-human organism, like a microorganism or a plant cell, plant or part thereof, especially with non-targeted localization. For example, an increase of the palmitic acid of at least 1 percent, particularly in a range of 18 to 31-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a purine nucleoside phosphorylase, or if the activity of the polypeptide AvinDRAFT_{—}6700, preferably represented by SEQ ID NO. 34045, or a homolog or fragment thereof, or if the activity of a polypeptide encoded by a nucleic acid molecule comprising the nucleic acid SEQ ID NO. 34044, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 34044 or polypeptide SEQ ID NO. 34045, respectively, or a homolog or a fragment thereof, is increased or generated, or if the activity purine nucleoside phosphorylase is increased or generated in a non-human organism, like a microorganism or a plant cell, plant or part thereof, especially with non-targeted localization. For example, an increase of the palmitic acid of at least 1 percent, particularly in a range of 21 to 29-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a b0050-protein, or if the activity of the polypeptide B0050, preferably represented by SEQ ID NO. 103959, or a homolog or fragment thereof, or if the activity of a polypeptide encoded by a nucleic acid molecule comprising the nucleic acid SEQ ID NO. 103958, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 103958 or polypeptide SEQ ID NO. 103959, respectively, or a homolog or a fragment thereof, is increased or generated, or if the activity b0050-protein is increased or generated in a non-human organism, like a microorganism or a plant cell, plant or part thereof, especially with non-targeted localization. For example, an increase of the palmitic acid of at least 1 percent, particularly in a range of 16 to 27-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a sodium/proton antiporter, or if the activity of the polypeptide B1186, preferably represented by SEQ ID NO. 37401, or a homolog or fragment thereof, or if the activity of a polypeptide encoded by a nucleic acid molecule comprising the nucleic acid SEQ ID NO. 37400, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 37400 or polypeptide SEQ ID NO. 37401, respectively, or a homolog or a fragment thereof, is increased or generated, or if the activity sodium/proton antiporter is increased or generated in a non-human organism, like a microorganism or a plant cell, plant or part thereof, especially with non-targeted localization. For example, an increase of the palmitic acid of at least 1 percent, particularly in a range of 20 to 47-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a b1234-protein, or if the activity of the polypeptide B1234, preferably represented by SEQ ID NO. 112112, or a homolog or fragment thereof, or if the activity of a polypeptide encoded by a nucleic acid molecule comprising the nucleic acid SEQ ID NO. 112111, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 112111 or polypeptide SEQ ID NO. 112112, respectively, or a homolog or a fragment thereof, is increased or generated, or if the activity b1234-protein is increased or generated in a non-human organism, like a microorganism or a plant cell, plant or part thereof, especially with non-targeted localization. For example, an increase of the palmitic acid of at least 1 percent, particularly in a range of 16 to 22-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a b1248-protein, or if the activity of the polypeptide B1248, preferably represented by SEQ ID NO. 119136, or a homolog or fragment thereof, or if the activity of a polypeptide encoded by a nucleic acid molecule comprising the nucleic acid SEQ ID NO. 119135, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 119135 or polypeptide SEQ ID NO. 119136, respectively, or a homolog or a fragment thereof, is increased or generated, or if the activity b1248-protein is increased or generated in a non-human organism, like a microorganism or a plant cell, plant or part thereof, especially with non-targeted localization. For example, an increase of the palmitic acid of at least 1 percent, particularly in a range of 15 to 22-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a b1330-protein, or if the activity of the polypeptide B1330, preferably represented by SEQ ID NO. 38227, or a homolog or fragment thereof, or if the activity of a polypeptide encoded by a nucleic acid molecule comprising the nucleic acid SEQ ID NO. 38226, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 38226 or polypeptide SEQ ID NO. 38227, respectively, or a homolog or a fragment thereof, is increased or generated, or if the activity b1330-protein is increased or generated in a non-human organism, like a microorganism or a plant cell, plant or part thereof, especially with non-targeted localization. For example, an increase of the palmitic acid of at least 1 percent, particularly in a range of 25 to 36-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a lipoprotein precursor, or if the activity of the polypeptide B1431, preferably represented by SEQ ID NO. 38267, or a homolog or fragment thereof, or if the activity of a polypeptide encoded by a nucleic acid molecule comprising the nucleic acid SEQ ID NO. 38266, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 38266 or polypeptide SEQ ID NO. 38267, respectively, or a homolog or a fragment thereof, is increased or generated, or if the activity lipoprotein precursor is increased or generated in a non-human organism, like a microorganism or a plant cell, plant or part thereof, especially with plastidic localization. For example, an increase of the palmitic acid of at least 1 percent, particularly in a range of 16 to 25-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a Fe—S subunit of oxidoreductase, or if the activity of the polypeptide B1589, preferably represented by SEQ ID NO. 78155, or a homolog or fragment thereof, or if the activity of a polypeptide encoded by a nucleic acid molecule comprising the nucleic acid SEQ ID NO. 78154, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 78154 or polypeptide SEQ ID NO. 78155, respectively, or a homolog or a fragment thereof, is increased or generated, or if the activity Fe—S subunit of oxidoreductase is increased or generated in a non-human organism, like a microorganism or a plant cell, plant or part thereof, especially with non-targeted localization. For example, an increase of the palmitic acid of at least 1 percent, particularly in a range of 16 to 25-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a acid shock protein, or if the activity of the polypeptide B1597, preferably represented by SEQ ID NO. 38301, or a homolog or fragment thereof, or if the activity of a polypeptide encoded by a nucleic acid molecule comprising the nucleic acid SEQ ID NO. 38300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 38300 or polypeptide SEQ ID NO. 38301, respectively, or a homolog or a fragment thereof, is increased or generated, or if the activity acid shock protein is increased or generated in a non-human organism, like a microorganism or a plant cell, plant or part thereof, especially with non-targeted localization. For example, an increase of the palmitic acid of at least 1 percent, particularly in a range of 20 to 44-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a b1670-protein, or if the activity of the polypeptide B1670, preferably represented by SEQ ID NO. 78754, or a homolog or fragment thereof, or if the activity of a polypeptide encoded by a nucleic acid molecule comprising the nucleic acid SEQ ID NO. 78753, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 78753 or polypeptide SEQ ID NO. 78754, respectively, or a homolog or a fragment thereof, is increased or generated, or if the activity b1670-protein is increased or generated in a non-human organism, like a microorganism or a plant cell, plant or part thereof, especially with non-targeted localization. For example, an increase of the palmitic acid of at least 1 percent, particularly in a range of 16 to 37-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a b1837-protein, or if the activity of the polypeptide B1837, preferably represented by SEQ ID NO. 78954, or a homolog or fragment thereof, or if the activity of a polypeptide encoded by a nucleic acid molecule comprising the nucleic acid SEQ ID NO. 78953, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 78953 or polypeptide SEQ ID NO. 78954, respectively, or a homolog or a fragment thereof, is increased or generated, or if the activity b1837-protein is increased or generated in a non-human organism, like a microorganism or a plant cell, plant or part thereof, especially with non-targeted localization. For example, an increase of the palmitic acid of at least 1 percent, particularly in a range of 16 to 39-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a b2032-protein, or if the activity of the polypeptide B2032, preferably represented by SEQ ID NO. 8921, or a homolog or fragment thereof, or if the activity of a polypeptide encoded by a nucleic acid molecule comprising the nucleic acid SEQ ID NO. 8920, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 8920 or polypeptide SEQ ID NO. 8921, respectively, or a homolog or a fragment thereof, is increased or generated, or if the activity b2032-protein is increased or generated in a non-human organism, like a microorganism or a plant cell, plant or part thereof, especially with non-targeted localization. For example, an increase of the palmitic acid of at least 1 percent, particularly in a range of 22 to 83-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a colanic acid biosynthesis protein, or if the activity of the polypeptide B2043, preferably represented by SEQ ID NO. 112194, or a homolog or fragment thereof, or if the activity of a polypeptide encoded by a nucleic acid molecule comprising the nucleic acid SEQ ID NO. 112193, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 112193 or polypeptide SEQ ID NO. 112194, respectively, or a homolog or a fragment thereof, is increased or generated, or if the activity colanic acid biosynthesis protein is increased or generated in a non-human organism, like a microorganism or a plant cell, plant or part thereof, especially with non-targeted localization. For example, an increase of the palmitic acid of at least 1 percent, particularly in a range of 16 to 54-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a b2099-protein, or if the activity of the polypeptide B2099, preferably represented by SEQ ID NO. 115596, or a homolog or fragment thereof, or if the activity of a polypeptide encoded by a nucleic acid molecule comprising the nucleic acid SEQ ID NO. 115595, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 115595 or polypeptide SEQ ID NO. 115596, respectively, or a homolog or a fragment thereof, is increased or generated, or if the activity b2099-protein is increased or generated in a non-human organism, like a microorganism or a plant cell, plant or part thereof, especially with non-targeted localization. For example, an increase of the palmitic acid of at least 1 percent, particularly in a range of 18 to 27-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a b2107-protein, or if the activity of the polypeptide B2107, preferably represented by SEQ ID NO. 39003, or a homolog or fragment thereof, or if the activity of a polypeptide encoded by a nucleic acid molecule comprising the nucleic acid SEQ ID NO. 39002, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 39002 or polypeptide SEQ ID NO. 39003, respectively, or a homolog or a fragment thereof, is increased or generated, or if the activity b2107-protein is increased or generated in a non-human organism, like a microorganism or a plant cell, plant or part thereof, especially with non-targeted localization. For example, an increase of the palmitic acid of at least 1 percent, particularly in a range of 15 to 37-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a b2399-protein, or if the activity of the polypeptide B2399, preferably represented by SEQ ID NO. 39238, or a homolog or fragment thereof, or if the activity of a polypeptide encoded by a nucleic acid molecule comprising the nucleic acid SEQ ID NO. 39237, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 39237 or polypeptide SEQ ID NO. 39238, respectively, or a homolog or a fragment thereof, is increased or generated, or if the activity b2399-protein is increased or generated in a non-human organism, like a microorganism or a plant cell, plant or part thereof, especially with non-targeted localization. For example, an increase of the palmitic acid of at least 1 percent, particularly in a range of 19 to 45-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a ethanolamine utilization protein, or if the activity of the polypeptide B2439, preferably represented by SEQ ID NO. 115629, or a homolog or fragment thereof, or if the activity of a polypeptide encoded by a nucleic acid molecule comprising the nucleic acid SEQ ID NO. 115628, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 115628 or polypeptide SEQ ID NO. 115629, respectively, or a homolog or a fragment thereof, is increased or generated, or if the activity ethanolamine utilization protein is increased or generated in a non-human organism, like a microorganism or a plant cell, plant or part thereof, especially with non-targeted localization. For example, an increase of the palmitic acid of at least 1 percent, particularly in a range of 16 to 34-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a b2682-protein, or if the activity of the polypeptide B2682, preferably represented by SEQ ID NO. 115656, or a homolog or fragment thereof, or if the activity of a polypeptide encoded by a nucleic acid molecule comprising the nucleic acid SEQ ID NO. 115655, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 115655 or polypeptide SEQ ID NO. 115656, respectively, or a homolog or a fragment thereof, is increased or generated, or if the activity b2682-protein is increased or generated in a non-human organism, like a microorganism or a plant cell, plant or part thereof, especially with non-targeted localization. For example, an increase of the palmitic acid of at least 1 percent, particularly in a range of 22 to 28-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a 2-C-methyl-D-erythritol 4-phosphate cytidylyltransferase, or if the activity of the polypeptide B2747, preferably represented by SEQ ID NO. 81076, or a homolog or fragment thereof, or if the activity of a polypeptide encoded by a nucleic acid molecule comprising the nucleic acid SEQ ID NO. 81075, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 81075 or polypeptide SEQ ID NO. 81076, respectively, or a homolog or a fragment thereof, is increased or generated, or if the activity 2-C-methyl-D-erythritol 4-phosphate cytidylyltransferase is increased or generated in a non-human organism, like a microorganism or a plant cell, plant or part thereof, especially with non-targeted localization. For example, an increase of the palmitic acid of at least 1 percent, particularly in a range of 23 to 38-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a flavodoxin, or if the activity of the polypeptide B2790, preferably represented by SEQ ID NO. 115720, or a homolog or fragment thereof, or if the activity of a polypeptide encoded by a nucleic acid molecule comprising the nucleic acid SEQ ID NO. 115719, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 115719 or polypeptide SEQ ID NO. 115720, respectively, or a homolog or a fragment thereof, is increased or generated, or if the activity flavodoxin is increased or generated in a non-human organism, like a microorganism or a plant cell, plant or part thereof, especially with non-targeted localization. For example, an increase of the palmitic acid of at least 1 percent, particularly in a range of 17 to 34-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a AAS bifunctional protein, or if the activity of the polypeptide B2836, preferably represented by SEQ ID NO. 115792, or a homolog or fragment thereof, or if the activity of a polypeptide encoded by a nucleic acid molecule comprising the nucleic acid SEQ ID NO. 115791, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 115791 or polypeptide SEQ ID NO. 115792, respectively, or a homolog or a fragment thereof, is increased or generated, or if the activity AAS bifunctional protein is increased or generated in a non-human organism, like a microorganism or a plant cell, plant or part thereof, especially with plastidic localization. For example, an increase of the palmitic acid of at least 1 percent, particularly in a range of 17 to 32-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a b2849-protein, or if the activity of the polypeptide B2849, preferably represented by SEQ ID NO. 81981, or a homolog or fragment thereof, or if the activity of a polypeptide encoded by a nucleic acid molecule comprising the nucleic acid SEQ ID NO. 81980, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 81980 or polypeptide SEQ ID NO. 81981, respectively, or a homolog or a fragment thereof, is increased or generated, or if the activity b2849-protein is increased or generated in a non-human organism, like a microorganism or a plant cell, plant or part thereof, especially with non-targeted localization. For example, an increase of the palmitic acid of at least 1 percent, particularly in a range of 20 to 56-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a b3814-protein, or if the activity of the polypeptide B3814, preferably represented by SEQ ID NO. 44197, or a homolog or fragment thereof, or if the activity of a polypeptide encoded by a nucleic acid molecule comprising the nucleic acid SEQ ID NO. 44196, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 44196 or polypeptide SEQ ID NO. 44197, respectively, or a homolog or a fragment thereof, is increased or generated, or if the activity b3814-protein is increased or generated in a non-human organism, like a microorganism or a plant cell, plant or part thereof, especially with non-targeted localization. For example, an increase of the palmitic acid of at least 1 percent, particularly in a range of 19 to 41-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a threonine efflux protein, or if the activity of the polypeptide B3823, preferably represented by SEQ ID NO. 10727, or a homolog or fragment thereof, or if the activity of a polypeptide encoded by a nucleic acid molecule comprising the nucleic acid SEQ ID NO. 10726, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 10726 or polypeptide SEQ ID NO. 10727, respectively, or a homolog or a fragment thereof, is increased or generated, or if the activity threonine efflux protein is increased or generated in a non-human organism, like a microorganism or a plant cell, plant or part thereof, especially with non-targeted localization. For example, an increase of the palmitic acid of at least 1 percent, particularly in a range of 21 to 35-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a 3-ketoacyl-CoA thiolase, or if the activity of the polypeptide B3845, preferably represented by SEQ ID NO. 100676, or a homolog or fragment thereof, or if the activity of a polypeptide encoded by a nucleic acid molecule comprising the nucleic acid SEQ ID NO. 100675, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 100675 or polypeptide SEQ ID NO. 100676, respectively, or a homolog or a fragment thereof, is increased or generated, or if the activity 3-ketoacyl-CoA thiolase is increased or generated in a non-human organism, like a microorganism or a plant cell, plant or part thereof, especially with plastidic localization. For example, an increase of the palmitic acid of at least 1 percent, particularly in a range of 15 to 31-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a glutaredoxin, or if the activity of the polypeptide GM02LC12622, preferably represented by SEQ ID NO. 10812, or a homolog or fragment thereof, or if the activity of a polypeptide encoded by a nucleic acid molecule comprising the nucleic acid SEQ ID NO. 10811, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 10811 or polypeptide SEQ ID NO. 10812, respectively, or a homolog or a fragment thereof, is increased or generated, or if the activity glutaredoxin is increased or generated in a non-human organism, like a microorganism or a plant cell, plant or part thereof, especially with non-targeted localization. For example, an increase of the palmitic acid of at least 1 percent, particularly in a range of 19 to 45-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a transcription factor, or if the activity of the polypeptide GM02LC19289, preferably represented by SEQ ID NO. 46752, or a homolog or fragment thereof, or if the activity of a polypeptide encoded by a nucleic acid molecule comprising the nucleic acid SEQ ID NO. 46751, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 46751 or polypeptide SEQ ID NO. 46752, respectively, or a homolog or a fragment thereof, is increased or generated, or if the activity transcription factor is increased or generated in a non-human organism, like a microorganism or a plant cell, plant or part thereof, especially with non-targeted localization. For example, an increase of the palmitic acid of at least 1 percent, particularly in a range of 17 to 80-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a bifunctional protein (phosphoribosyltransferase and regulatory protein), or if the activity of the polypeptide Sll0368, preferably represented by SEQ ID NO. 106921, or a homolog or fragment thereof, or if the activity of a polypeptide encoded by a nucleic acid molecule comprising the nucleic acid SEQ ID NO. 106920, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 106920 or polypeptide SEQ ID NO. 106921, respectively, or a homolog or a fragment thereof, is increased or generated, or if the activity bifunctional protein (phosphoribosyltransferase and regulatory protein) is increased or generated in a non-human organism, like a microorganism or a plant cell, plant or part thereof, especially with mitochondrial localization. For example, an increase of the palmitic acid of at least 1 percent, particularly in a range of 20 to 28-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a branched chain amino acid ABC transporter ATP-binding protein, or if the activity of the polypeptide Sll0374, preferably represented by SEQ ID NO. 94742, or a homolog or fragment thereof, or if the activity of a polypeptide encoded by a nucleic acid molecule comprising the nucleic acid SEQ ID NO. 94741, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 94741 or polypeptide SEQ ID NO. 94742, respectively, or a homolog or a fragment thereof, is increased or generated, or if the activity branched chain amino acid ABC transporter ATP-binding protein is increased or generated in a non-human organism, like a microorganism or a plant cell, plant or part thereof, especially with plastidic localization. For example, an increase of the palmitic acid of at least 1 percent, particularly in a range of 20 to 23-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a carbon dioxide concentrating mechanism protein, or if the activity of the polypeptide Sll1031, preferably represented by SEQ ID NO. 52635, or a homolog or fragment thereof, or if the activity of a polypeptide encoded by a nucleic acid molecule comprising the nucleic acid SEQ ID NO. 52634, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, 11 or IV, application No. 11, column 5 or 8 in the respective same line as the nucleic acid molecule SEQ ID NO. 52634 or polypeptide SEQ ID NO. 52635, respectively, or a homolog or a fragment thereof, is increased or generated, or if the activity carbon dioxide concentrating mechanism protein is increased or generated in a non-human organism, like a microorganism or a plant cell, plant or part thereof, especially with mitochondrial localization. For example, an increase of the palmitic acid of at least 1 percent, particularly in a range of 18 to 38-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a sll1032-protein, or if the activity of the polypeptide Sll1032, preferably represented by SEQ ID NO. 85724, or a homolog or fragment thereof, or if the activity of a polypeptide encoded by a nucleic acid molecule comprising the nucleic acid SEQ ID NO. 85723, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 85723 or polypeptide SEQ ID NO. 85724, respectively, or a homolog or a fragment thereof, is increased or generated, or if the activity sll1032-protein is increased or generated in a non-human organism, like a microorganism or a plant cell, plant or part thereof, especially with mitochondrial localization. For example, an increase of the palmitic acid of at least 1 percent, particularly in a range of 16 to 27-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a phosphoribosylformyl glycinamidine synthase subunit, or if the activity of the poylpeptide Sll1056, preferably represented by SEQ ID NO. 52661, or a homolog or fragment thereof, or if the activity of a polypeptide encoded by a nucleic acid molecule comprising the nucleic acid SEQ ID NO. 52660, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 52660 or polypeptide SEQ ID NO. 52661, respectively, or a homolog or a fragment thereof, is increased or generated, or if the activity phosphoribosylformyl glycinamidine synthase subunit is increased or generated in a non-human organism, like a microorganism or a plant cell, plant or part thereof, especially with plastidic localization. For example, an increase of the palmitic acid of at least 1 percent, particularly in a range of 21 to 38-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a nitrate/nitrite transport protein, or if the activity of the polypeptide Sll1450, preferably represented by SEQ ID NO. 54338, or a homolog or fragment thereof, or if the activity of a polypeptide encoded by a nucleic acid molecule comprising the nucleic acid SEQ ID NO. 54337, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 54337 or polypeptide SEQ ID NO. 54338, respectively, or a homolog or a fragment thereof, is increased or generated, or if the activity nitrate/nitrite transport protein is increased or generated in a non-human organism, like a microorganism or a plant cell, plant or part thereof, especially with mitochondrial localization. For example, an increase of the palmitic acid of at least 1 percent, particularly in a range of 19 to 29-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a carbamoylphosphate synthase subunit, or if the activity of the polypeptide Sll1498, preferably represented by SEQ ID NO. 95048, or a homolog or fragment thereof, or if the activity of a polypeptide encoded by a nucleic acid molecule comprising the nucleic acid SEQ ID NO. 95047, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 95047 or polypeptide SEQ ID NO. 95048, respectively, or a homolog or a fragment thereof, is increased or generated, or if the activity carbamoyl-phosphate synthase subunit is increased or generated in a non-human organism, like a microorganism or a plant cell, plant or part thereof, especially with non-targeted localization. For example, an increase of the palmitic acid of at least 1 percent, particularly in a range of 18 to 24-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a glutathione S-transferase, or if the activity of the polypeptide Sll1545, preferably represented by SEQ ID NO. 11424, or a homolog or fragment thereof, or if the activity of a polypeptide encoded by a nucleic acid molecule comprising the nucleic acid SEQ ID NO. 11423, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 11423 or polypeptide SEQ ID NO. 11424, respectively, or a homolog or a fragment thereof, is increased or generated, or if the activity glutathione S-transferase is increased or generated in a non-human organism, like a microorganism or a plant cell, plant or part thereof, especially with mitochondrial localization. For example, an increase of the palmitic acid of at least 1 percent, particularly in a range of 15 to 27-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a ABC metal ion transporter substrate-binding protein, or if the activity of the polypeptide Sll1598, preferably represented by SEQ ID NO. 85744, or a homolog or fragment thereof, or if the activity of a polypeptide encoded by a nucleic acid molecule comprising the nucleic acid SEQ ID NO. 85743, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 85743 or polypeptide SEQ ID NO. 85744, respectively, or a homolog or a fragment thereof, is increased or generated, or if the activity ABC metal ion transporter substrate-binding protein is increased or generated in a non-human organism, like a microorganism or a plant cell, plant or part thereof, especially with mitochondrial localization. For example, an increase of the palmitic acid of at least 1 percent, particularly in a range of 17 to 28-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a acyl transferase, or if the activity of the poylpeptide Sll1848, preferably represented by SEQ ID NO. 86448, or a homolog or fragment thereof, or if the activity of a polypeptide encoded by a nucleic acid molecule comprising the nucleic acid SEQ ID NO. 86447, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 86447 or polypeptide SEQ ID NO. 86448, respectively, or a homolog or a fragment thereof, is increased or generated, or if the activity acyl transferase is increased or generated in a non-human organism, like a microorganism or a plant cell, plant or part thereof, especially with plastidic localization. For example, an increase of the palmitic acid of at least 1 percent, particularly in a range of 18 to 115-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a coproporphyrinogen III oxidase, or if the activity of the polypeptide Sll1917, preferably represented by SEQ ID NO. 11472, or a homolog or fragment thereof, or if the activity of a polypeptide encoded by a nucleic acid molecule comprising the nucleic acid SEQ ID NO. 11471, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, 11 or IV, application No. 11, column 5 or 8 in the respective same line as the nucleic acid molecule SEQ ID NO. 11471 or polypeptide SEQ ID NO. 11472, respectively, or a homolog or a fragment thereof, is increased or generated, or if the activity coproporphyrinogen III oxidase is increased or generated in a non-human organism, like a microorganism or a plant cell, plant or part thereof, especially with mitochondrial localization. For example, an increase of the palmitic acid of at least 1 percent, particularly in a range of 16 to 23-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a cationtransporting ATPase, or if the activity of the polypeptide Sll1920, preferably represented by SEQ ID NO. 55386, or a homolog or fragment thereof, or if the activity of a polypeptide encoded by a nucleic acid molecule comprising the nucleic acid SEQ ID NO. 55385, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 55385 or polypeptide SEQ ID NO. 55386, respectively, or a homolog or a fragment thereof, is increased or generated, or if the activity cation-transporting ATPase is increased or generated in a non-human organism, like a microorganism or a plant cell, plant or part thereof, especially with mitochondrial localization. For example, an increase of the palmitic acid of at least 1 percent, particularly in a range of 18 to 34-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a permease protein of ABC transporter, or if the activity of the polypeptide Slr0949, preferably represented by SEQ ID NO. 119223, or a homolog or fragment thereof, or if the activity of a polypeptide encoded by a nucleic acid molecule comprising the nucleic acid SEQ ID NO. 119222, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 119222 or polypeptide SEQ ID NO. 119223, respectively, or a homolog or a fragment thereof, is increased or generated, or if the activity permease protein of ABC transporter is increased or generated in a non-human organism, like a microorganism or a plant cell, plant or part thereof, especially with mitochondrial localization. For example, an increase of the palmitic acid of at least 1 percent, particularly in a range of 18 to 25-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a aromatic acid decarboxylase, or if the activity of the polypeptide Slr1099, preferably represented by SEQ ID NO. 116342, or a homolog or fragment thereof, or if the activity of a polypeptide encoded by a nucleic acid molecule comprising the nucleic acid SEQ ID NO. 116341, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 116341 or polypeptide SEQ ID NO. 116342, respectively, or a homolog or a fragment thereof, is increased or generated, or if the activity aromatic acid decarboxylase is increased or generated in a non-human organism, like a microorganism or a plant cell, plant or part thereof, especially with mitochondrial localization. For example, an increase of the palmitic acid of at least 1 percent, particularly in a range of 23 to 39-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a phosphate import ATP-binding protein, or if the activity of the polypeptide Slr1250, preferably represented by SEQ ID NO. 116461, or a homolog or fragment thereof, or if the activity of a polypeptide encoded by a nucleic acid molecule comprising the nucleic acid SEQ ID NO. 116460, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 116460 or polypeptide SEQ ID NO. 116461, respectively, or a homolog or a fragment thereof, is increased or generated, or if the activity phosphate import ATP-binding protein is increased or generated in a non-human organism, like a microorganism or a plant cell, plant or part thereof, especially with mitochondrial localization. For example, an increase of the palmitic acid of at least 1 percent, particularly in a range of 19 to 33-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a arginine decarboxylase, or if the activity of the polypeptide Slr1312, preferably represented by SEQ ID NO. 58325, or a homolog or fragment thereof, or if the activity of a polypeptide encoded by a nucleic acid molecule comprising the nucleic acid SEQ ID NO. 58324, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 58324 or polypeptide SEQ ID NO. 58325, respectively, or a homolog or a fragment thereof, is increased or generated, or if the activity arginine decarboxylase is increased or generated in a non-human organism, like a microorganism or a plant cell, plant or part thereof, especially with non-targeted localization. For example, an increase of the palmitic acid of at least 1 percent, particularly in a range of 21 to 34-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a phosphoadenosine phosphosulfate reductase , or if the activity of the polypeptide Slr1791, preferably represented by SEQ ID NO. 12141, or a homolog or fragment thereof, or if the activity of a polypeptide encoded by a nucleic acid molecule comprising the nucleic acid SEQ ID NO. 12140, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 12140 or polypeptide SEQ ID NO. 12141, respectively, or a homolog or a fragment thereof, is increased or generated, or if the activity phosphoadenosine phosphosulfate reductase is increased or generated in a non-human organism, like a microorganism or a plant cell, plant or part thereof, especially with mitochondrial localization. For example, an increase of the palmitic acid of at least 1 percent, particularly in a range of 16 to 17-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a threonine dehydratase, or if the activity of the polypeptide Slr2072, preferably represented by SEQ ID NO. 12342, or a homolog or fragment thereof, or if the activity of a polypeptide encoded by a nucleic acid molecule comprising the nucleic acid SEQ ID NO. 12341, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 12341 or polypeptide SEQ ID NO. 12342, respectively, or a homolog or a fragment thereof, is increased or generated, or if the activity threonine dehydratase is increased or generated in a non-human organism, like a microorganism or a plant cell, plant or part thereof, especially with mitochondrial localization. For example, an increase of the palmitic acid of at least 1 percent, particularly in a range of 19 to 42-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a metaldependent hydrolase, or if the activity of the polypeptide TTC0917, preferably represented by SEQ ID NO. 61554, or a homolog or fragment thereof, or if the activity of a polypeptide encoded by a nucleic acid molecule comprising the nucleic acid SEQ ID NO. 61553, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 61553 or polypeptide SEQ ID NO. 61554, respectively, or a homolog or a fragment thereof, is increased or generated, or if the activity metal-dependent hydrolase is increased or generated in a non-human organism, like a microorganism or a plant cell, plant or part thereof, especially with non-targeted localization. For example, an increase of the palmitic acid of at least 1 percent, particularly in a range of 16 to 112-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a homocitrate synthase, or if the activity of the polypeptide TTC1550, preferably represented by SEQ ID NO. 12975, or a homolog or fragment thereof, or if the activity of a polypeptide encoded by a nucleic acid molecule comprising the nucleic acid SEQ ID NO. 12974, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 12974 or polypeptide SEQ ID NO. 12975, respectively, or a homolog or a fragment thereof, is increased or generated, or if the activity homocitrate synthase is increased or generated in a non-human organism, like a microorganism or a plant cell, plant or part thereof, especially with non-targeted localization. For example, an increase of the palmitic acid of at least 1 percent, particularly in a range of 19 to 35-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a acid phosphatase precursor, or if the activity of the polypeptide Ybr092c, preferably represented by SEQ ID NO. 119348, or a homolog or fragment thereof, or if the activity of a polypeptide encoded by a nucleic acid molecule comprising the nucleic acid SEQ ID NO. 119347, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 119347 or polypeptide SEQ ID NO. 119348, respectively, or a homolog or a fragment thereof, is increased or generated, or if the activity acid phosphatase precursor is increased or generated in a non-human organism, like a microorganism or a plant cell, plant or part thereof, especially with plastidic localization. For example, an increase of the palmitic acid of at least 1 percent, particularly in a range of 16 to 31-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a glycerol-3-phosphate dehydrogenase, or if the activity of the polypeptide Ydl022w, preferably represented by SEQ ID NO. 119409, or a homolog or fragment thereof, or if the activity of a polypeptide encoded by a nucleic acid molecule comprising the nucleic acid SEQ ID NO. 119408, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 119408 or polypeptide SEQ ID NO. 119409, respectively, or a homolog or a fragment thereof, is increased or generated, or if the activity glycerol-3-phosphate dehydrogenase is increased or generated in a non-human organism, like a microorganism or a plant cell, plant or part thereof, especially with non-targeted localization. For example, an increase of the palmitic acid of at least 1 percent, particularly in a range of 13 to 39-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a protein kinase, or if the activity of the polypeptide Ydl101c, preferably represented by SEQ ID NO. 122519, or a homolog or fragment thereof, or if the activity of a polypeptide encoded by a nucleic acid molecule comprising the nucleic acid SEQ ID NO. 122518, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 122518 or polypeptide SEQ ID NO. 122519, respectively, or a homolog or a fragment thereof, is increased or generated, or if the activity protein kinase is increased or generated in a non-human organism, like a microorganism or a plant cell, plant or part thereof, especially with non-targeted localization. For example, an increase of the palmitic acid of at least 1 percent, particularly in a range of 20 to 36-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a F-box protein, or if the activity of the polypeptide Ydr131c, preferably represented by SEQ ID NO. 14707, or a homolog or fragment thereof, or if the activity of a polypeptide encoded by a nucleic acid molecule comprising the nucleic acid SEQ ID NO. 14706, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 14706 or polypeptide SEQ ID NO. 14707, respectively, or a homolog or a fragment thereof, is increased or generated, or if the activity F-box protein is increased or generated in a non-human organism, like a microorganism or a plant cell, plant or part thereof, especially with plastidic localization. For example, an increase of the palmitic acid of at least 1 percent, particularly in a range of 19 to 37-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a DnaJ-like chaperone, or if the activity of the polypeptide Yjl073w, preferably represented by SEQ ID NO. 64965, or a homolog or fragment thereof, or if the activity of a polypeptide encoded by a nucleic acid molecule comprising the nucleic acid SEQ ID NO. 64964, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 64964 or polypeptide SEQ ID NO. 64965, respectively, or a homolog or a fragment thereof, is increased or generated, or if the activity DnaJ-like chaperone is increased or generated in a non-human organism, like a microorganism or a plant cell, plant or part thereof, especially with plastidic localization. For example, an increase of the palmitic acid of at least 1 percent, particularly in a range of 18 to 39-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a aconitate hydratase, or if the activity of the polypeptide Ylr304c, preferably represented by SEQ ID NO. 66773, or a homolog or fragment thereof, or if the activity of a polypeptide encoded by a nucleic acid molecule comprising the nucleic acid SEQ ID NO. 66772, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 66772 or polypeptide SEQ ID NO. 66773, respectively, or a homolog or a fragment thereof, is increased or generated, or if the activity aconitate hydratase is increased or generated in a non-human organism, like a microorganism or a plant cell, plant or part thereof, especially with non-targeted localization. For example, an increase of the palmitic acid of at least 1 percent, particularly in a range of 16 to 38-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a ymr013cprotein, or if the activity of the polypeptide Ymr013c, preferably represented by SEQ ID NO. 91959, or a homolog or fragment thereof, or if the activity of a polypeptide encoded by a nucleic acid molecule comprising the nucleic acid SEQ ID NO. 91958, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 91958 or polypeptide SEQ ID NO. 91959, respectively, or a homolog or a fragment thereof, is increased or generated, or if the activity ymr013c-protein is increased or generated in a non-human organism, like a microorganism or a plant cell, plant or part thereof, especially with plastidic localization. For example, an increase of the palmitic acid of at least 1 percent, particularly in a range of 16 to 26-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a ATPase epsilon subunit, or if the activity of the polypeptide Ypl271w, preferably represented by SEQ ID NO. 118213, or a homolog or fragment thereof, or if the activity of a polypeptide encoded by a nucleic acid molecule comprising the nucleic acid SEQ ID NO. 118212, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 118212 or polypeptide SEQ ID NO. 118213, respectively, or a homolog or a fragment thereof, is increased or generated, or if the activity ATPase epsilon subunit is increased or generated in a non-human organism, like a microorganism or a plant cell, plant or part thereof, especially with plastidic localization. For example, an increase of the palmitic acid of at least 1 percent, particularly in a range of 19 to 33-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a 49747384_SOYBEAN-protein, or if the activity of the polypeptide 49747384_SOYBEAN, preferably represented by SEQ ID NO. 70, or a homolog or fragment thereof, or if the activity of a polypeptide encoded by a nucleic acid molecule comprising the nucleic acid SEQ ID NO. 69, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 69 or polypeptide SEQ ID NO. 70, respectively, or a homolog or a fragment thereof, is increased or generated, or if the activity 49747384_SOYBEAN-protein is increased or generated in a non-human organism, like a microorganism or a plant cell, plant or part thereof, especially with non-targeted localization. For example, an increase of the palmitic acid of at least 1 percent, particularly in a range of 15 to 29-percent is conferred as compared to a corresponding non-transformed wild type non-human organism.

Accordingly, in one embodiment, an increase of palmitic acid in a non-human organism, as compared to a corresponding non-transformed wild type non-human organism, is conferred in the process of the invention, if the activity of a polypeptide showing the activity of a glucose-6-phosphate 1-dehydrogenase, or if the activity of the polypeptide 59582753_SOYBEAN, preferably represented by SEQ ID NO. 16884, or a homolog or fragment thereof, or if the activity of a polypeptide encoded by a nucleic acid molecule comprising the nucleic acid SEQ ID NO. 16883, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 11, column 5 or 8 in the respective same line as the nucleic acid molecule SEQ ID NO. 16883 or polypeptide SEQ ID NO. 16884, respectively, or a homolog or a fragment thereof, is increased or generated, or if the activity glucose-6-phosphate 1-dehydrogenase is increased or generated in a non-human organism, like a microorganism or a plant cell, plant or part thereof, especially with plastidic localization. For example, an increase of the palmitic acid of at least 1 percent, particularly in a range of 16 to 42-percent is conferred as compared to a corresponding non-transformed wild type non-human organism.

[0096.1.8.11] to [0103.1.8.11] for the disclosure of these paragraphs see [0096.1.8.8] to [0103.1.8.8] above.

In this paragraph “A” means “Goffeau et al., Science 274 (5287), 546 (1996)”, “B” means “Blattner et al., Science 277 (5331), 1453 (1997)”, “C” means “Nakamura et al., Nucleic Acids Res. 28(1), 72 (2000)”, “D” means “deposition of the sequences to the EMBL/GenBank/DDBJ databases under “Sequencing of the draft genome assembly of Azotobacter vinelandii AvOP”. in June 2005 by Copeland et al.”, “E” means “Henne et al., Nat. Biotechnol. 22 (5), 547 (2004)”, “F” means “Kaul et al., Nature 408 (6814), 796 (2000)”, and “G” means “Matsumoto et al., Nature 436 (7052), 793 (2005)”.

The nucleic acid sequence of At1g07430 from Arabidopsis thaliana, e.g. as shown in the respective line in column 5 of Table I, application no. 11, has been published in F. And the activity of the gene product thereof is the activity of protein phosphatase.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said At1g07430,or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said At1g07430, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said At1g07430, or a functional equivalent or a homolog thereof as depicted in column 8 of Table II, application no. 11, preferably a homolog or functional equivalent as depicted in column 8 of Table II B, application no. 11, and being depicted in the same respective line as said At1g07430, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 17451, preferably the coding region thereof, conferred the production of or the increase in palmitic acid compared with the wild type control.

The nucleic acid sequence of At1g19670 from Arabidopsis thaliana, e.g. as shown in the respective line in column 5 of Table I, application no. 11, has been published in F. And the activity of the gene product thereof is the activity of chlorophyllase.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “chlorophyllase”, especially from Arabidopsis thaliana or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said At1g19670, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said At1g19670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said At1g19670, or a functional equivalent or a homolog thereof as depicted in column 8 of Table II, application no. 11, preferably a homolog or functional equivalent as depicted in column 8 of Table II B, application no. 11, and being depicted in the same respective line as said At1g19670, and preferably the activity is increased non-targeted.

Accordingly, in one embodiment, the molecule which activity is to be increased in the process of the invention is the gene product with an activity as a “chlorophyllase”, preferably it is the molecule of section (a) or (b) of this paragraph.

In particular, it was observed that in plants, especially in Arabidopsis thaliana, increasing or generating the activity of a gene product non-targeted with the activity of a “chlorophyllase”, preferably being encoded by a gene comprising the nucleic acid sequence SEQ ID NO. 113651, preferably the coding region thereof, conferred the production of or the increase in palmitic acid compared with the wild type control.

The nucleic acid sequence of At1g19800 from Arabidopsis thaliana, e.g. as shown in the respective line in column 5 of Table I, application no. 11, has been published in F. And the activity of the gene product thereof is the activity of At1g19800-protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “At1g19800-protein”, especially from Arabidopsis thaliana or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said At1g19800, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said At1g19800, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said At1g19800, or a functional equivalent or a homolog thereof as depicted in column 8 of Table II, application no. 11, preferably a homolog or functional equivalent as depicted in column 8 of Table II B, application no. 11, and being depicted in the same respective line as said At1g19800, and preferably the activity is increased non-targeted.

Accordingly, in one embodiment, the molecule which activity is to be increased in the process of the invention is the gene product with an activity as a “At1g19800-protein”, preferably it is the molecule of section (a) or (b) of this paragraph.

In particular, it was observed that in plants, especially in Arabidopsis thaliana, increasing or generating the activity of a gene product non-targeted with the activity of a “At1g19800-protein”, preferably being encoded by a gene comprising the nucleic acid sequence SEQ ID NO. 17637, preferably the coding region thereof, conferred the production of or the increase in palmitic acid compared with the wild type control.

The nucleic acid sequence of At1g23110 from Arabidopsis thaliana, e.g. as shown in the respective line in column 5 of Table I, application no. 11, has been published in F. And the activity of the gene product thereof is the activity of At1g23110-protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “At1g23110-protein”, especially from Arabidopsis thaliana or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said At1g23110, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said At1g23110, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said At1g23110, or a functional equivalent or a homolog thereof as depicted in column 8 of Table II, application no. 11, preferably a homolog or functional equivalent as depicted in column 8 of Table II B, application no. 11, and being depicted in the same respective line as said At1g23110, and preferably the activity is increased non-targeted.

Accordingly, in one embodiment, the molecule which activity is to be increased in the process of the invention is the gene product with an activity as a “At1g23110-protein”, preferably it is the molecule of section (a) or (b) of this paragraph.

In particular, it was observed that in plants, especially in Arabidopsis thaliana, increasing or generating the activity of a gene product non-targeted with the activity of a “At1g23110-protein”, preferably being encoded by a gene comprising the nucleic acid sequence SEQ ID NO. 102086, preferably the coding region thereof, conferred the production of or the increase in palmitic acid compared with the wild type control.

The nucleic acid sequence of At1g68320 from Arabidopsis thaliana, e.g. as shown in the respective line in column 5 of Table I, application no. 11, has been published in F. And the activity of the gene product thereof is the activity of transcription factor.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said At1g68320, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said At1g68320, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said At1g68320, or a functional equivalent or a homolog thereof as depicted in column 8 of Table II, application no. 11, preferably a homolog or functional equivalent as depicted in column 8 of Table II B, application no. 11, and being depicted in the same respective line as said At1g68320, and preferably the activity is increased non-targeted.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 1061, preferably the coding region thereof, conferred the production of or the increase in palmitic acid compared with the wild type control.

The nucleic acid sequence of At1g72770 from Arabidopsis thaliana, e.g. as shown in the respective line in column 5 of Table I, application no. 11, has been published in F. And the activity of the gene product thereof is the activity of protein phosphatase.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said At1g72770, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said At1g72770, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said At1g72770, or a functional equivalent or a homolog thereof as depicted in column 8 of Table II, application no. 11, preferably a homolog or functional equivalent as depicted in column 8 of Table II B, application no. 11, and being depicted in the same respective line as said At1g72770, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19419, preferably the coding region thereof, conferred the production of or the increase in palmitic acid compared with the wild type control.

The nucleic acid sequence of At2g45420 from Arabidopsis thaliana, e.g. as shown in the respective line in column 5 of Table I, application no. 11, has been published in F. And the activity of the gene product thereof is the activity of At2g45420-protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “At2g45420-protein”, especially from Arabidopsis thaliana or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said At2g45420, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said At2g45420, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said At2g45420, or a functional equivalent or a homolog thereof as depicted in column 8 of Table II, application no. 11, preferably a homolog or functional equivalent as depicted in column 8 of Table II B, application no. 11, and being depicted in the same respective line as said At2g45420, and preferably the activity is increased non-targeted.

Accordingly, in one embodiment, the molecule which activity is to be increased in the process of the invention is the gene product with an activity as a “At2g45420-protein”, preferably it is the molecule of section (a) or (b) of this paragraph.

In particular, it was observed that in plants, especially in Arabidopsis thaliana, increasing or generating the activity of a gene product non-targeted with the activity of a “At2g45420-protein”, preferably being encoded by a gene comprising the nucleic acid sequence SEQ ID NO. 1623, preferably the coding region thereof, conferred the production of or the increase in palmitic acid compared with the wild type control.

The nucleic acid sequence of At3g02990 from Arabidopsis thaliana, e.g. as shown in the respective line in column 5 of Table I, application no. 11, has been published in F. And the activity of the gene product thereof is the activity of heat shock transcription factor.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said At3g02990, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said At3g02990, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said At3g02990, or a functional equivalent or a homolog thereof as depicted in column 8 of Table II, application no. 11, preferably a homolog or functional equivalent as depicted in column 8 of Table II B, application no. 11, and being depicted in the same respective line as said At3g02990, and preferably the activity is increased non-targeted.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 68657, preferably the coding region thereof, conferred the production of or the increase in palmitic acid compared with the wild type control.

The nucleic acid sequence of At3g20910 from Arabidopsis thaliana, e.g. as shown in the respective line in column 5 of Table I, application no. 11, has been published in F. And the activity of the gene product thereof is the activity of CCAAT-binding transcription factor.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “CCAAT-binding transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said At3g20910, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said At3g20910, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said At3g20910, or a functional equivalent or a homolog thereof as depicted in column 8 of Table II, application no. 11, preferably a homolog or functional equivalent as depicted in column 8 of Table II B, application no. 11, and being depicted in the same respective line as said At3g20910, and preferably the activity is increased non-targeted.

Accordingly, in one embodiment, the molecule which activity is to be increased in the process of the invention is the gene product with an activity as a “CCAAT-binding transcription factor”, preferably it is the molecule of section (a) or (b) of this paragraph.

In particular, it was observed that in plants, especially in Arabidopsis thaliana, increasing or generating the activity of a gene product non-targeted with the activity of a “CCAAT-binding transcription factor”, preferably being encoded by a gene comprising the nucleic acid sequence SEQ ID NO. 22921, preferably the coding region thereof, conferred the production of or the increase in palmitic acid compared with the wild type control.

The nucleic acid sequence of At3g62950 from Arabidopsis thaliana, e.g. as shown in the respective line in column 5 of Table I, application no. 11, has been published in F. And the activity of the gene product thereof is the activity of glutaredoxin.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said At3g62950, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said At3g62950, or a functional equivalent or a homolog thereof as depicted in column 8 of Table II, application no. 11, preferably a homolog or functional equivalent as depicted in column 8 of Table II B, application no. 11, and being depicted in the same respective line as said At3g62950, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in palmitic acid compared with the wild type control.

The nucleic acid sequence of At4g15670 from Arabidopsis thaliana, e.g. as shown in the respective line in column 5 of Table I, application no. 11, has been published in F. And the activity of the gene product thereof is the activity of monothiol glutaredoxin.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said At4g15670, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said At4g15670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said At4g15670, or a functional equivalent or a homolog thereof as depicted in column 8 of Table II, application no. 11, preferably a homolog or functional equivalent as depicted in column 8 of Table II B, application no. 11, and being depicted in the same respective line as said At4g15670, and preferably the activity is increased non-targeted.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3279, preferably the coding region thereof, conferred the production of or the increase in palmitic acid compared with the wild type control.

The nucleic acid sequence of At4g18880 from Arabidopsis thaliana, e.g. as shown in the respective line in column 5 of Table I, application no. 11, has been published in F. And the activity of the gene product thereof is the activity of heat shock transcription factor.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said At4g18880, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said At4g18880, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said At4g18880, or a functional equivalent or a homolog thereof as depicted in column 8 of Table II, application no. 11, preferably a homolog or functional equivalent as depicted in column 8 of Table II B, application no. 11, and being depicted in the same respective line as said At4g18880, and preferably the activity is increased non-targeted.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 24232, preferably the coding region thereof, conferred the production of or the increase in palmitic acid compared with the wild type control.

The nucleic acid sequence of At4g26080 from Arabidopsis thaliana, e.g. as shown in the respective line in column 5 of Table I, application no. 11, has been published in F. And the activity of the gene product thereof is the activity of protein phosphatase.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said At4g26080, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said At4g26080, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said At4g26080, or a functional equivalent or a homolog thereof as depicted in column 8 of Table II, application no. 11, preferably a homolog or functional equivalent as depicted in column 8 of Table II B, application no. 11, and being depicted in the same respective line as said At4g26080, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 68849, preferably the coding region thereof, conferred the production of or the increase in palmitic acid compared with the wild type control.

The nucleic acid sequence of At4g33040 from Arabidopsis thaliana, e.g. as shown in the respective line in column 5 of Table I, application no. 11, has been published in F. And the activity of the gene product thereof is the activity of glutaredoxin.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said At4g33040, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said At4g33040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said At4g33040, or a functional equivalent or a homolog thereof as depicted in column 8 of Table II, application no. 11, preferably a homolog or functional equivalent as depicted in column 8 of Table II B, application no. 11, and being depicted in the same respective line as said At4g33040, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 4102, preferably the coding region thereof, conferred the production of or the increase in palmitic acid compared with the wild type control.

The nucleic acid sequence of At4g35310 from Arabidopsis thaliana, e.g. as shown in the respective line in column 5 of Table I, application no. 11, has been published in F. And the activity of the gene product thereof is the activity of calcium-dependent protein kinase.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said At4g35310, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said At4g35310, or a functional equivalent or a homolog thereof as depicted in column 8 of Table II, application no. 11, preferably a homolog or functional equivalent as depicted in column 8 of Table II B, application no. 11, and being depicted in the same respective line as said At4g35310, and preferably the activity is increased non-targeted.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in palmitic acid compared with the wild type control.

The nucleic acid sequence of At5g57050 from Arabidopsis thaliana, e.g. as shown in the respective line in column 5 of Table I, application no. 11, has been published in F. And the activity of the gene product thereof is the activity of protein phosphatase.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said At5g57050, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said At5g57050, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said At5g57050, or a functional equivalent or a homolog thereof as depicted in column 8 of Table II, application no. 11, preferably a homolog or functional equivalent as depicted in column 8 of Table II B, application no. 11, and being depicted in the same respective line as said At5g57050, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 5318, preferably the coding region thereof, conferred the production of or the increase in palmitic acid compared with the wild type control.

The nucleic acid sequence of At5g59220 from Arabidopsis thaliana, e.g. as shown in the respective line in column 5 of Table I, application no. 11, has been published in F. And the activity of the gene product thereof is the activity of protein phosphatase.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said At5g59220, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said At5g59220, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said At5g59220, or a functional equivalent or a homolog thereof as depicted in column 8 of Table II, application no. 11, preferably a homolog or functional equivalent as depicted in column 8 of Table II B, application no. 11, and being depicted in the same respective line as said At5g59220, and preferably the activity is increased non-targeted.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 25498, preferably the coding region thereof, conferred the production of or the increase in palmitic acid compared with the wild type control.

The nucleic acid sequence of AvinDRAFT_{—}1788 from Azotobacter vinelandii, e.g. as shown in the respective line in column 5 of Table I, application no. 11, has been published in D. And the activity of the gene product thereof is the activity of oxidoreductase.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “oxidoreductase”, especially from Azotobacter vinelandii or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said AvinDRAFT_{—}1788, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said AvinDRAFT_{—}1788, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said AvinDRAFT_{—}1788, or a functional equivalent or a homolog thereof as depicted in column 8 of Table II, application no. 11, preferably a homolog or functional equivalent as depicted in column 8 of Table II B, application no. 11, and being depicted in the same respective line as said AvinDRAFT_{—}1788, and preferably the activity is increased non-targeted.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 114083, preferably the coding region thereof, conferred the production of or the increase in palmitic acid compared with the wild type control.

The nucleic acid sequence of AvinDRAFT_{—}3577 from Azotobacter vinelandii, e.g. as shown in the respective line in column 5 of Table I, application no. 11, has been published in D. And the activity of the gene product thereof is the activity of ThiF family protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “ThiF family protein”, especially from Azotobacter vinelandii or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said AvinDRAFT_{—}3577, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said AvinDRAFT_{—}3577, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said AvinDRAFT_{—}3577, or a functional equivalent or a homolog thereof as depicted in column 8 of Table II, application no. 11, preferably a homolog or functional equivalent as depicted in column 8 of Table II B, application no. 11, and being depicted in the same respective line as said AvinDRAFT_{—}3577, and preferably the activity is increased non-targeted.

Accordingly, in one embodiment, the molecule which activity is to be increased in the process of the invention is the gene product with an activity as a “ThiF family protein”, preferably it is the molecule of section (a) or (b) of this paragraph.

In particular, it was observed that in plants, especially in Arabidopsis thaliana, increasing or generating the activity of a gene product non-targeted with the activity of a “ThiF family protein”, preferably being encoded by a gene comprising the nucleic acid sequence SEQ ID NO. 111155, preferably the coding region thereof, conferred the production of or the increase in palmitic acid compared with the wild type control.

The nucleic acid sequence of AvinDRAFT_{—}3629 from Azotobacter vinelandii, e.g. as shown in the respective line in column 5 of Table I, application no. 11, has been published in D. And the activity of the gene product thereof is the activity of recombinase A.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “recombinase A”, especially from Azotobacter vinelandii or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said AvinDRAFT_{—}3629, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said AvinDRAFT_{—}3629, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said AvinDRAFT_{—}3629, or a functional equivalent or a homolog thereof as depicted in column 8 of Table II, application no. 11, preferably a homolog or functional equivalent as depicted in column 8 of Table II B, application no. 11, and being depicted in the same respective line as said AvinDRAFT_{—}3629, and preferably the activity is increased non-targeted.

Accordingly, in one embodiment, the molecule which activity is to be increased in the process of the invention is the gene product with an activity as a “recombinase A”, preferably it is the molecule of section (a) or (b) of this paragraph.

In particular, it was observed that in plants, especially in Arabidopsis thaliana, increasing or generating the activity of a gene product non-targeted with the activity of a “recombinase A”, preferably being encoded by a gene comprising the nucleic acid sequence SEQ ID NO. 114231, preferably the coding region thereof, conferred the production of or the increase in palmitic acid compared with the wild type control.

The nucleic acid sequence of AvinDRAFT_{—}4606 from Azotobacter vinelandii, e.g. as shown in the respective line in column 5 of Table I, application no. 11, has been published in D. And the activity of the gene product thereof is the activity of NADH-quinone oxidoreductase subunit.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “NADH-quinone oxidoreductase subunit”, especially from Azotobacter vinelandii or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said AvinDRAFT_{—}4606, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said AvinDRAFT_{—}4606, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said AvinDRAFT_{—}4606, or a functional equivalent or a homolog thereof as depicted in column 8 of Table II, application no. 11, preferably a homolog or functional equivalent as depicted in column 8 of Table II B, application no. 11, and being depicted in the same respective line as said AvinDRAFT_{—}4606, and preferably the activity is increased non-targeted.

Accordingly, in one embodiment, the molecule which activity is to be increased in the process of the invention is the gene product with an activity as a “NADH-quinone oxidoreductase subunit”, preferably it is the molecule of section (a) or (b) of this paragraph.

In particular, it was observed that in plants, especially in Arabidopsis thaliana, increasing or generating the activity of a gene product non-targeted with the activity of a “NADH-quinone oxidoreductase subunit”, preferably being encoded by a gene comprising the nucleic acid sequence SEQ ID NO. 73719, preferably the coding region thereof, conferred the production of or the increase in palmitic acid compared with the wild type control.

The nucleic acid sequence of AvinDRAFT_{—}5467 from Azotobacter vinelandii, e.g. as shown in the respective line in column 5 of Table I, application no. 11, has been published in D. And the activity of the gene product thereof is the activity of HesB/YadR/YfhF family protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “HesB/YadR/YfhF family protein”, especially from Azotobacter vinelandii or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said AvinDRAFT_{—}5467, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said AvinDRAFT_{—}5467, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said AvinDRAFT_{—}5467, or a functional equivalent or a homolog thereof as depicted in column 8 of Table II, application no. 11, preferably a homolog or functional equivalent as depicted in column 8 of Table II B, application no. 11, and being depicted in the same respective line as said AvinDRAFT_{—}5467, and preferably the activity is increased non-targeted.

Accordingly, in one embodiment, the molecule which activity is to be increased in the process of the invention is the gene product with an activity as a “HesB/YadR/YfhF family protein”, preferably it is the molecule of section (a) or (b) of this paragraph.

In particular, it was observed that in plants, especially in Arabidopsis thaliana, increasing or generating the activity of a gene product non-targeted with the activity of a “HesB/YadR/YfhF family protein”, preferably being encoded by a gene comprising the nucleic acid sequence SEQ ID NO. 32648, preferably the coding region thereof, conferred the production of or the increase in palmitic acid compared with the wild type control.

The nucleic acid sequence of AvinDRAFT_{—}6075 from Azotobacter vinelandii, e.g. as shown in the respective line in column 5 of Table I, application no. 11, has been published in D. And the activity of the gene product thereof is the activity of peptidyl-prolyl cis-trans isomerase.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “peptidyl-prolyl cis-trans isomerase”, especially from Azotobacter vinelandii or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said AvinDRAFT_{—}6075, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said AvinDRAFT_{—}6075, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said AvinDRAFT_{—}6075, or a functional equivalent or a homolog thereof as depicted in column 8 of Table II, application no. 11, preferably a homolog or functional equivalent as depicted in column 8 of Table II B, application no. 11, and being depicted in the same respective line as said AvinDRAFT_{—}6075, and preferably the activity is increased non-targeted.

Accordingly, in one embodiment, the molecule which activity is to be increased in the process of the invention is the gene product with an activity as a “peptidyl-prolyl cis-trans isomerase”, preferably it is the molecule of section (a) or (b) of this paragraph.

In particular, it was observed that in plants, especially in Arabidopsis thaliana, increasing or generating the activity of a gene product non-targeted with the activity of a “peptidyl-prolyl cis-trans isomerase”, preferably being encoded by a gene comprising the nucleic acid sequence SEQ ID NO. 6674, preferably the coding region thereof, conferred the production of or the increase in palmitic acid compared with the wild type control.

The nucleic acid sequence of AvinDRAFT_{—}6700 from Azotobacter vinelandii, e.g. as shown in the respective line in column 5 of Table I, application no. 11, has been published in D. And the activity of the gene product thereof is the activity of purine nucleoside phosphorylase.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “purine nucleoside phosphorylase”, especially from Azotobacter vinelandii or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said AvinDRAFT_{—}6700, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said AvinDRAFT_{—}6700, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said AvinDRAFT_{—}6700, or a functional equivalent or a homolog thereof as depicted in column 8 of Table II, application no. 11, preferably a homolog or functional equivalent as depicted in column 8 of Table II B, application no. 11, and being depicted in the same respective line as said AvinDRAFT_{—}6700, and preferably the activity is increased non-targeted.

Accordingly, in one embodiment, the molecule which activity is to be increased in the process of the invention is the gene product with an activity as a “purine nucleoside phosphorylase”, preferably it is the molecule of section (a) or (b) of this paragraph.

In particular, it was observed that in plants, especially in Arabidopsis thaliana, increasing or generating the activity of a gene product non-targeted with the activity of a “purine nucleoside phosphorylase”, preferably being encoded by a gene comprising the nucleic acid sequence SEQ ID NO. 34044, preferably the coding region thereof, conferred the production of or the increase in palmitic acid compared with the wild type control.

The nucleic acid sequence of B0050 from Escherichia coli, e.g. as shown in the respective line in column 5 of Table I, application no. 11, has been published in B. And the activity of the gene product thereof is the activity of b0050-protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “b0050-protein”, especially from Escherichia coli or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said B0050, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said B0050, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said B0050, or a functional equivalent or a homolog thereof as depicted in column 8 of Table II, application no. 11, preferably a homolog or functional equivalent as depicted in column 8 of Table II B, application no. 11, and being depicted in the same respective line as said B0050, and preferably the activity is increased non-targeted.

Accordingly, in one embodiment, the molecule which activity is to be increased in the process of the invention is the gene product with an activity as a “b0050-protein”, preferably it is the molecule of section (a) or (b) of this paragraph.

In particular, it was observed that in plants, especially in Arabidopsis thaliana, increasing or generating the activity of a gene product non-targeted with the activity of a “b0050-protein”, preferably being encoded by a gene comprising the nucleic acid sequence SEQ ID NO. 103958, preferably the coding region thereof, conferred the production of or the increase in palmitic acid compared with the wild type control.

The nucleic acid sequence of B1186 from Escherichia coli, e.g. as shown in the respective line in column 5 of Table I, application no. 11, has been published in B. And the activity of the gene product thereof is the activity of sodium/proton antiporter.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “sodium/proton antiporter”, especially from Escherichia coli or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said B1186, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said B1186, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said B1186, or a functional equivalent or a homolog thereof as depicted in column 8 of Table II, application no. 11, preferably a homolog or functional equivalent as depicted in column 8 of Table II B, application no. 11, and being depicted in the same respective line as said B1186, and preferably the activity is increased non-targeted.

Accordingly, in one embodiment, the molecule which activity is to be increased in the process of the invention is the gene product with an activity as a “sodium/proton antiporter”, preferably it is the molecule of section (a) or (b) of this paragraph.

In particular, it was observed that in plants, especially in Arabidopsis thaliana, increasing or generating the activity of a gene product non-targeted with the activity of a “sodium/proton antiporter”, preferably being encoded by a gene comprising the nucleic acid sequence SEQ ID NO. 37400, preferably the coding region thereof, conferred the production of or the increase in palmitic acid compared with the wild type control.

The nucleic acid sequence of B1234 from Escherichia coli, e.g. as shown in the respective line in column 5 of Table I, application no. 11, has been published in B. And the activity of the gene product thereof is the activity of b1234-protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “b1234-protein”, especially from Escherichia coli or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said B1234, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said B1234, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said B1234, or a functional equivalent or a homolog thereof as depicted in column 8 of Table II, application no. 11, preferably a homolog or functional equivalent as depicted in column 8 of Table II B, application no. 11, and being depicted in the same respective line as said B1234, and preferably the activity is increased non-targeted.

Accordingly, in one embodiment, the molecule which activity is to be increased in the process of the invention is the gene product with an activity as a “b1234-protein”, preferably it is the molecule of section (a) or (b) of this paragraph.

In particular, it was observed that in plants, especially in Arabidopsis thaliana, increasing or generating the activity of a gene product non-targeted with the activity of a “b1234-protein”, preferably being encoded by a gene comprising the nucleic acid sequence SEQ ID NO. 112111, preferably the coding region thereof, conferred the production of or the increase in palmitic acid compared with the wild type control.

The nucleic acid sequence of B1248 from Escherichia coli, e.g. as shown in the respective line in column 5 of Table I, application no. 11, has been published in B. And the activity of the gene product thereof is the activity of b1248-protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “b1248-protein”, especially from Escherichia coli or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said B1248, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said B1248, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said B1248, or a functional equivalent or a homolog thereof as depicted in column 8 of Table II, application no. 11, preferably a homolog or functional equivalent as depicted in column 8 of Table II B, application no. 11, and being depicted in the same respective line as said B1248, and preferably the activity is increased non-targeted.

Accordingly, in one embodiment, the molecule which activity is to be increased in the process of the invention is the gene product with an activity as a “b1248-protein”, preferably it is the molecule of section (a) or (b) of this paragraph.

In particular, it was observed that in plants, especially in Arabidopsis thaliana, increasing or generating the activity of a gene product non-targeted with the activity of a “b1248-protein”, preferably being encoded by a gene comprising the nucleic acid sequence SEQ ID NO. 119135, preferably the coding region thereof, conferred the production of or the increase in palmitic acid compared with the wild type control.

The nucleic acid sequence of B1330 from Escherichia coli, e.g. as shown in the respective line in column 5 of Table I, application no. 11, has been published in B. And the activity of the gene product thereof is the activity of b1330-protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “b1330-protein”, especially from Escherichia coli or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said B1330, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said B1330, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said B1330, or a functional equivalent or a homolog thereof as depicted in column 8 of Table II, application no. 11, preferably a homolog or functional equivalent as depicted in column 8 of Table II B, application no. 11, and being depicted in the same respective line as said B1330, and preferably the activity is increased non-targeted.

Accordingly, in one embodiment, the molecule which activity is to be increased in the process of the invention is the gene product with an activity as a “b1330-protein”, preferably it is the molecule of section (a) or (b) of this paragraph.

In particular, it was observed that in plants, especially in Arabidopsis thaliana, increasing or generating the activity of a gene product non-targeted with the activity of a “b1330-protein”, preferably being encoded by a gene comprising the nucleic acid sequence SEQ ID NO. 38226, preferably the coding region thereof, conferred the production of or the increase in palmitic acid compared with the wild type control.

The nucleic acid sequence of B1431 from Escherichia coli, e.g. as shown in the respective line in column 5 of Table I, application no. 11, has been published in B. And the activity of the gene product thereof is the activity of lipoprotein precursor.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “lipoprotein precursor”, especially from Escherichia coli or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said B1431, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said B1431, and preferably the activity is increased plastidic, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said B1431, or a functional equivalent or a homolog thereof as depicted in column 8 of Table II, application no. 11, preferably a homolog or functional equivalent as depicted in column 8 of Table II B, application no. 11, and being depicted in the same respective line as said B1431, and preferably the activity is increased plastidic.

Accordingly, in one embodiment, the molecule which activity is to be increased in the process of the invention is the gene product with an activity as a “lipoprotein precursor”, preferably it is the molecule of section (a) or (b) of this paragraph.

In particular, it was observed that in plants, especially in Arabidopsis thaliana, increasing or generating the activity of a gene product plastidic with the activity of a “lipoprotein precursor”, preferably being encoded by a gene comprising the nucleic acid sequence SEQ ID NO. 38266, preferably the coding region thereof, conferred the production of or the increase in palmitic acid compared with the wild type control.

The nucleic acid sequence of B1589 from Escherichia coli, e.g. as shown in the respective line in column 5 of Table I, application no. 11, has been published in B. And the activity of the gene product thereof is the activity of Fe—S subunit of oxidoreductase.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “Fe—S subunit of oxidoreductase”, especially from Escherichia coli or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said B1589, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said B1589, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said B1589, or a functional equivalent or a homolog thereof as depicted in column 8 of Table II, application no. 11, preferably a homolog or functional equivalent as depicted in column 8 of Table II B, application no. 11, and being depicted in the same respective line as said B1589, and preferably the activity is increased non-targeted.

Accordingly, in one embodiment, the molecule which activity is to be increased in the process of the invention is the gene product with an activity as a “Fe—S subunit of oxidoreductase”, preferably it is the molecule of section (a) or (b) of this paragraph.

In particular, it was observed that in plants, especially in Arabidopsis thaliana, increasing or generating the activity of a gene product non-targeted with the activity of a “Fe—S subunit of oxidoreductase”, preferably being encoded by a gene comprising the nucleic acid sequence SEQ ID NO. 78154, preferably the coding region thereof, conferred the production of or the increase in palmitic acid compared with the wild type control.

The nucleic acid sequence of B1597 from Escherichia coli, e.g. as shown in the respective line in column 5 of Table I, application no. 11, has been published in B. And the activity of the gene product thereof is the activity of acid shock protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “acid shock protein”, especially from Escherichia coli or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said B1597, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said B1597, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said B1597, or a functional equivalent or a homolog thereof as depicted in column 8 of Table II, application no. 11, preferably a homolog or functional equivalent as depicted in column 8 of Table II B, application no. 11, and being depicted in the same respective line as said B1597, and preferably the activity is increased non-targeted.

Accordingly, in one embodiment, the molecule which activity is to be increased in the process of the invention is the gene product with an activity as a “acid shock protein”, preferably it is the molecule of section (a) or (b) of this paragraph.

In particular, it was observed that in plants, especially in Arabidopsis thaliana, increasing or generating the activity of a gene product non-targeted with the activity of a “acid shock protein”, preferably being encoded by a gene comprising the nucleic acid sequence SEQ ID NO. 38300, preferably the coding region thereof, conferred the production of or the increase in palmitic acid compared with the wild type control.

The nucleic acid sequence of B1670 from Escherichia coli, e.g. as shown in the respective line in column 5 of Table I, application no. 11, has been published in B. And the activity of the gene product thereof is the activity of b1670-protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “b1670-protein”, especially from Escherichia coli or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said B1670, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said B1670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said B1670, or a functional equivalent or a homolog thereof as depicted in column 8 of Table II, application no. 11, preferably a homolog or functional equivalent as depicted in column 8 of Table II B, application no. 11, and being depicted in the same respective line as said B1670, and preferably the activity is increased non-targeted.

Accordingly, in one embodiment, the molecule which activity is to be increased in the process of the invention is the gene product with an activity as a “b1670-protein”, preferably it is the molecule of section (a) or (b) of this paragraph.

In particular, it was observed that in plants, especially in Arabidopsis thaliana, increasing or generating the activity of a gene product non-targeted with the activity of a “b1670-protein”, preferably being encoded by a gene comprising the nucleic acid sequence SEQ ID NO. 78753, preferably the coding region thereof, conferred the production of or the increase in palmitic acid compared with the wild type control.

The nucleic acid sequence of B1837 from Escherichia coli, e.g. as shown in the respective line in column 5 of Table I, application no. 11, has been published in B. And the activity of the gene product thereof is the activity of b1837-protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “b1837-protein”, especially from Escherichia coli or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said B1837, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said B1837, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said B1837, or a functional equivalent or a homolog thereof as depicted in column 8 of Table II, application no. 11, preferably a homolog or functional equivalent as depicted in column 8 of Table II B, application no. 11, and being depicted in the same respective line as said B1837, and preferably the activity is increased non-targeted.

Accordingly, in one embodiment, the molecule which activity is to be increased in the process of the invention is the gene product with an activity as a “b1837-protein”, preferably it is the molecule of section (a) or (b) of this paragraph.

In particular, it was observed that in plants, especially in Arabidopsis thaliana, increasing or generating the activity of a gene product non-targeted with the activity of a “b1837-protein”, preferably being encoded by a gene comprising the nucleic acid sequence SEQ ID NO. 78953, preferably the coding region thereof, conferred the production of or the increase in palmitic acid compared with the wild type control.

The nucleic acid sequence of B2032 from Escherichia coli, e.g. as shown in the respective line in column 5 of Table I, application no. 11, has been published in B. And the activity of the gene product thereof is the activity of b2032-protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “b2032-protein”, especially from Escherichia coli or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said B2032, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said B2032, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said B2032, or a functional equivalent or a homolog thereof as depicted in column 8 of Table II, application no. 11, preferably a homolog or functional equivalent as depicted in column 8 of Table II B, application no. 11, and being depicted in the same respective line as said B2032, and preferably the activity is increased non-targeted.

Accordingly, in one embodiment, the molecule which activity is to be increased in the process of the invention is the gene product with an activity as a “b2032-protein”, preferably it is the molecule of section (a) or (b) of this paragraph.

In particular, it was observed that in plants, especially in Arabidopsis thaliana, increasing or generating the activity of a gene product non-targeted with the activity of a “b2032-protein”, preferably being encoded by a gene comprising the nucleic acid sequence SEQ ID NO. 8920, preferably the coding region thereof, conferred the production of or the increase in palmitic acid compared with the wild type control.

The nucleic acid sequence of B2043 from Escherichia coli, e.g. as shown in the respective line in column 5 of Table I, application no. 11, has been published in B. And the activity of the gene product thereof is the activity of colanic acid biosynthesis protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “colanic acid biosynthesis protein”, especially from Escherichia coli or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said B2043, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said B2043, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said B2043, or a functional equivalent or a homolog thereof as depicted in column 8 of Table II, application no. 11, preferably a homolog or functional equivalent as depicted in column 8 of Table II B, application no. 11, and being depicted in the same respective line as said B2043, and preferably the activity is increased non-targeted.

Accordingly, in one embodiment, the molecule which activity is to be increased in the process of the invention is the gene product with an activity as a “colanic acid biosynthesis protein”, preferably it is the molecule of section (a) or (b) of this paragraph.

In particular, it was observed that in plants, especially in Arabidopsis thaliana, increasing or generating the activity of a gene product non-targeted with the activity of a “colanic acid biosynthesis protein”, preferably being encoded by a gene comprising the nucleic acid sequence SEQ ID NO. 112193, preferably the coding region thereof, conferred the production of or the increase in palmitic acid compared with the wild type control.

The nucleic acid sequence of B2099 from Escherichia coli, e.g. as shown in the respective line in column 5 of Table I, application no. 11, has been published in B. And the activity of the gene product thereof is the activity of b2099-protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “b2099-protein”, especially from Escherichia coli or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said B2099, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said B2099, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said B2099, or a functional equivalent or a homolog thereof as depicted in column 8 of Table II, application no. 11, preferably a homolog or functional equivalent as depicted in column 8 of Table II B, application no. 11, and being depicted in the same respective line as said B2099, and preferably the activity is increased non-targeted.

Accordingly, in one embodiment, the molecule which activity is to be increased in the process of the invention is the gene product with an activity as a “b2099-protein”, preferably it is the molecule of section (a) or (b) of this paragraph.

In particular, it was observed that in plants, especially in Arabidopsis thaliana, increasing or generating the activity of a gene product non-targeted with the activity of a “b2099-protein”, preferably being encoded by a gene comprising the nucleic acid sequence SEQ ID NO. 115595, preferably the coding region thereof, conferred the production of or the increase in palmitic acid compared with the wild type control.

The nucleic acid sequence of B2107 from Escherichia coli, e.g. as shown in the respective line in column 5 of Table I, application no. 11, has been published in B. And the activity of the gene product thereof is the activity of b2107-protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “b2107-protein”, especially from Escherichia coli or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said B2107, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said B2107, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said B2107, or a functional equivalent or a homolog thereof as depicted in column 8 of Table II, application no. 11, preferably a homolog or functional equivalent as depicted in column 8 of Table II B, application no. 11, and being depicted in the same respective line as said B2107, and preferably the activity is increased non-targeted.

Accordingly, in one embodiment, the molecule which activity is to be increased in the process of the invention is the gene product with an activity as a “b2107-protein”, preferably it is the molecule of section (a) or (b) of this paragraph.

In particular, it was observed that in plants, especially in Arabidopsis thaliana, increasing or generating the activity of a gene product non-targeted with the activity of a “b2107-protein”, preferably being encoded by a gene comprising the nucleic acid sequence SEQ ID NO. 39002, preferably the coding region thereof, conferred the production of or the increase in palmitic acid compared with the wild type control.

The nucleic acid sequence of B2399 from Escherichia coli, e.g. as shown in the respective line in column 5 of Table I, application no. 11, has been published in B. And the activity of the gene product thereof is the activity of b2399-protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “b2399-protein”, especially from Escherichia coli or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said B2399, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said B2399, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said B2399, or a functional equivalent or a homolog thereof as depicted in column 8 of Table II, application no. 11, preferably a homolog or functional equivalent as depicted in column 8 of Table II B, application no. 11, and being depicted in the same respective line as said B2399, and preferably the activity is increased non-targeted.

Accordingly, in one embodiment, the molecule which activity is to be increased in the process of the invention is the gene product with an activity as a “b2399-protein”, preferably it is the molecule of section (a) or (b) of this paragraph.

In particular, it was observed that in plants, especially in Arabidopsis thaliana, increasing or generating the activity of a gene product non-targeted with the activity of a “b2399-protein”, preferably being encoded by a gene comprising the nucleic acid sequence SEQ ID NO. 39237, preferably the coding region thereof, conferred the production of or the increase in palmitic acid compared with the wild type control.

The nucleic acid sequence of B2439 from Escherichia coli, e.g. as shown in the respective line in column 5 of Table I, application no. 11, has been published in B. And the activity of the gene product thereof is the activity of ethanolamine utilization protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “ethanolamine utilization protein”, especially from Escherichia coli or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said B2439, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said B2439, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said B2439, or a functional equivalent or a homolog thereof as depicted in column 8 of Table II, application no. 11, preferably a homolog or functional equivalent as depicted in column 8 of Table II B, application no. 11, and being depicted in the same respective line as said B2439, and preferably the activity is increased non-targeted.

Accordingly, in one embodiment, the molecule which activity is to be increased in the process of the invention is the gene product with an activity as a “ethanolamine utilization protein”, preferably it is the molecule of section (a) or (b) of this paragraph.

In particular, it was observed that in plants, especially in Arabidopsis thaliana, increasing or generating the activity of a gene product non-targeted with the activity of a “ethanolamine utilization protein”, preferably being encoded by a gene comprising the nucleic acid sequence SEQ ID NO. 115628, preferably the coding region thereof, conferred the production of or the increase in palmitic acid compared with the wild type control.

The nucleic acid sequence of B2682 from Escherichia coli, e.g. as shown in the respective line in column 5 of Table I, application no. 11, has been published in B. And the activity of the gene product thereof is the activity of b2682-protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “b2682-protein”, especially from Escherichia coli or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said B2682, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said B2682, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said B2682, or a functional equivalent or a homolog thereof as depicted in column 8 of Table II, application no. 11, preferably a homolog or functional equivalent as depicted in column 8 of Table II B, application no. 11, and being depicted in the same respective line as said B2682, and preferably the activity is increased non-targeted.

Accordingly, in one embodiment, the molecule which activity is to be increased in the process of the invention is the gene product with an activity as a “b2682-protein”, preferably it is the molecule of section (a) or (b) of this paragraph.

In particular, it was observed that in plants, especially in Arabidopsis thaliana, increasing or generating the activity of a gene product non-targeted with the activity of a “b2682-protein”, preferably being encoded by a gene comprising the nucleic acid sequence SEQ ID NO. 115655, preferably the coding region thereof, conferred the production of or the increase in palmitic acid compared with the wild type control.

The nucleic acid sequence of B2747 from Escherichia coli, e.g. as shown in the respective line in column 5 of Table I, application no. 11, has been published in B. And the activity of the gene product thereof is the activity of 2-C-methyl-D-erythritol 4-phosphate cytidylyltransferase.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “2-C-methyl-D-erythritol 4-phosphate cytidylyltransferase”, especially from Escherichia coli or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said B2747, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said B2747, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said B2747, or a functional equivalent or a homolog thereof as depicted in column 8 of Table II, application no. 11, preferably a homolog or functional equivalent as depicted in column 8 of Table II B, application no. 11, and being depicted in the same respective line as said B2747, and preferably the activity is increased non-targeted.

Accordingly, in one embodiment, the molecule which activity is to be increased in the process of the invention is the gene product with an activity as a “2-C-methyl-D-erythritol 4-phosphate cytidylyltransferase”, preferably it is the molecule of section (a) or (b) of this paragraph.

In particular, it was observed that in plants, especially in Arabidopsis thaliana, increasing or generating the activity of a gene product non-targeted with the activity of a “2-C-methyl-D-erythritol 4-phosphate cytidylyltransferase”, preferably being encoded by a gene comprising the nucleic acid sequence SEQ ID NO. 81075, preferably the coding region thereof, conferred the production of or the increase in palmitic acid compared with the wild type control.

The nucleic acid sequence of B2790 from Escherichia coli, e.g. as shown in the respective line in column 5 of Table I, application no. 11, has been published in B. And the activity of the gene product thereof is the activity of flavodoxin.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “flavodoxin”, especially from Escherichia coli or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said B2790, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said B2790, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said B2790, or a functional equivalent or a homolog thereof as depicted in column 8 of Table II, application no. 11, preferably a homolog or functional equivalent as depicted in column 8 of Table II B, application no. 11, and being depicted in the same respective line as said B2790, and preferably the activity is increased non-targeted.

Accordingly, in one embodiment, the molecule which activity is to be increased in the process of the invention is the gene product with an activity as a “flavodoxin”, preferably it is the molecule of section (a) or (b) of this paragraph.

In particular, it was observed that in plants, especially in Arabidopsis thaliana, increasing or generating the activity of a gene product non-targeted with the activity of a “flavodoxin”, preferably being encoded by a gene comprising the nucleic acid sequence SEQ ID NO. 115719, preferably the coding region thereof, conferred the production of or the increase in palmitic acid compared with the wild type control.

The nucleic acid sequence of B2836 from Escherichia coli, e.g. as shown in the respective line in column 5 of Table I, application no. 11, has been published in B. And the activity of the gene product thereof is the activity of AAS bifunctional protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “AAS bifunctional protein”, especially from Escherichia coli or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said B2836, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said B2836, and preferably the activity is increased plastidic, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said B2836, or a functional equivalent or a homolog thereof as depicted in column 8 of Table II, application no. 11, preferably a homolog or functional equivalent as depicted in column 8 of Table II B, application no. 11, and being depicted in the same respective line as said B2836, and preferably the activity is increased plastidic.

Accordingly, in one embodiment, the molecule which activity is to be increased in the process of the invention is the gene product with an activity as a “AAS bifunctional protein”, preferably it is the molecule of section (a) or (b) of this paragraph.

In particular, it was observed that in plants, especially in Arabidopsis thaliana, increasing or generating the activity of a gene product plastidic with the activity of a “AAS bifunctional protein”, preferably being encoded by a gene comprising the nucleic acid sequence SEQ ID NO. 115791, preferably the coding region thereof, conferred the production of or the increase in palmitic acid compared with the wild type control.

The nucleic acid sequence of B2849 from Escherichia coli, e.g. as shown in the respective line in column 5 of Table I, application no. 11, has been published in B. And the activity of the gene product thereof is the activity of b2849-protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “b2849-protein”, especially from Escherichia coli or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said B2849, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said B2849, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said B2849, or a functional equivalent or a homolog thereof as depicted in column 8 of Table II, application no. 11, preferably a homolog or functional equivalent as depicted in column 8 of Table II B, application no. 11, and being depicted in the same respective line as said B2849, and preferably the activity is increased non-targeted.

Accordingly, in one embodiment, the molecule which activity is to be increased in the process of the invention is the gene product with an activity as a “b2849-protein”, preferably it is the molecule of section (a) or (b) of this paragraph.

In particular, it was observed that in plants, especially in Arabidopsis thaliana, increasing or generating the activity of a gene product non-targeted with the activity of a “b2849-protein”, preferably being encoded by a gene comprising the nucleic acid sequence SEQ ID NO. 81980, preferably the coding region thereof, conferred the production of or the increase in palmitic acid compared with the wild type control.

The nucleic acid sequence of B3814 from Escherichia coli, e.g. as shown in the respective line in column 5 of Table I, application no. 11, has been published in B. And the activity of the gene product thereof is the activity of b3814-protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “b3814-protein”, especially from Escherichia coli or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said B3814, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said B3814, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said B3814, or a functional equivalent or a homolog thereof as depicted in column 8 of Table II, application no. 11, preferably a homolog or functional equivalent as depicted in column 8 of Table II B, application no. 11, and being depicted in the same respective line as said B3814, and preferably the activity is increased non-targeted.

Accordingly, in one embodiment, the molecule which activity is to be increased in the process of the invention is the gene product with an activity as a “b3814-protein”, preferably it is the molecule of section (a) or (b) of this paragraph.

In particular, it was observed that in plants, especially in Arabidopsis thaliana, increasing or generating the activity of a gene product non-targeted with the activity of a “b3814-protein”, preferably being encoded by a gene comprising the nucleic acid sequence SEQ ID NO. 44196, preferably the coding region thereof, conferred the production of or the increase in palmitic acid compared with the wild type control.

The nucleic acid sequence of B3823 from Escherichia coli, e.g. as shown in the respective line in column 5 of Table I, application no. 11, has been published in B. And the activity of the gene product thereof is the activity of threonine efflux protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “threonine efflux protein”, especially from Escherichia coli or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said B3823, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said B3823, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said B3823, or a functional equivalent or a homolog thereof as depicted in column 8 of Table II, application no. 11, preferably a homolog or functional equivalent as depicted in column 8 of Table II B, application no. 11, and being depicted in the same respective line as said B3823, and preferably the activity is increased non-targeted.

Accordingly, in one embodiment, the molecule which activity is to be increased in the process of the invention is the gene product with an activity as a “threonine efflux protein”, preferably it is the molecule of section (a) or (b) of this paragraph.

In particular, it was observed that in plants, especially in Arabidopsis thaliana, increasing or generating the activity of a gene product non-targeted with the activity of a “threonine efflux protein”, preferably being encoded by a gene comprising the nucleic acid sequence SEQ ID NO. 10726, preferably the coding region thereof, conferred the production of or the increase in palmitic acid compared with the wild type control.

The nucleic acid sequence of B3845 from Escherichia coli, e.g. as shown in the respective line in column 5 of Table I, application no. 11, has been published in B. And the activity of the gene product thereof is the activity of 3-ketoacyl-CoA thiolase.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “3-ketoacyl-CoA thiolase”, especially from Escherichia coli or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said B3845, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said B3845, and preferably the activity is increased plastidic, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said B3845, or a functional equivalent or a homolog thereof as depicted in column 8 of Table II, application no. 11, preferably a homolog or functional equivalent as depicted in column 8 of Table II B, application no. 11, and being depicted in the same respective line as said B3845, and preferably the activity is increased plastidic.

Accordingly, in one embodiment, the molecule which activity is to be increased in the process of the invention is the gene product with an activity as a “3-ketoacyl-CoA thiolase”, preferably it is the molecule of section (a) or (b) of this paragraph.

In particular, it was observed that in plants, especially in Arabidopsis thaliana, increasing or generating the activity of a gene product plastidic with the activity of a “3-ketoacyl-CoA thiolase”, preferably being encoded by a gene comprising the nucleic acid sequence SEQ ID NO. 100675, preferably the coding region thereof, conferred the production of or the increase in palmitic acid compared with the wild type control.

The nucleic acid sequence of GM02LC12622 from Glycine max, e.g. as shown in the respective line in column 5 of Table I, application no. 11, is unpublished. And the activity of the gene product thereof is the activity of glutaredoxin.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “glutaredoxin”, especially from Glycine max or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said GM02LC12622, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said GM02LC12622, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said GM02LC12622, or a functional equivalent or a homolog thereof as depicted in column 8 of Table II, application no. 11, preferably a homolog or functional equivalent as depicted in column 8 of Table II B, application no. 11, and being depicted in the same respective line as said GM02LC12622, and preferably the activity is increased non-targeted.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 10811, preferably the coding region thereof, conferred the production of or the increase in palmitic acid compared with the wild type control.

The nucleic acid sequence of GM02LC19289 from Glycine max, e.g. as shown in the respective line in column 5 of Table I, application no. 11, is unpublished. And the activity of the gene product thereof is the activity of transcription factor.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said GM02LC19289, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said GM02LC19289, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said GM02LC19289, or a functional equivalent or a homolog thereof as depicted in column 8 of Table II, application no. 11, preferably a homolog or functional equivalent as depicted in column 8 of Table II B, application no. 11, and being depicted in the same respective line as said GM02LC19289, and preferably the activity is increased non-targeted.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 46751, preferably the coding region thereof, conferred the production of or the increase in palmitic acid compared with the wild type control.

The nucleic acid sequence of Sll0368 from Synechocystis sp., e.g. as shown in the respective line in column 5 of Table I, application no. 11, has been published in C. And the activity of the gene product thereof is the activity of bifunctional protein (phosphoribosyltransferase and regulatory protein).

Accordingly, in one embodiment, the process of the present invention for producing palmitic acid in a non-human organism, like a microorganism or a plant or a part thereof, comprises increasing or generating the activity of a gene product with the activity of a gene product conferring the activity of “bifunctional protein (phosphoribosyltransferase and regulatory protein)”, especially from Synechocystis sp. or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said Sll0368, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said Sll0368, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said Sll0368, or a functional equivalent or a homolog thereof as depicted in column 8 of Table II, application no. 11, preferably a homolog or functional equivalent as depicted in column 8 of Table II B, application no. 11, and being depicted in the same respective line as said Sll0368, and preferably the activity is increased mitochondrial.

Accordingly, in one embodiment, the molecule which activity is to be increased in the process of the invention is the gene product with an activity as a “bifunctional protein (phosphoribosyltransferase and regulatory protein)”, preferably it is the molecule of section (a) or (b) of this paragraph.

In particular, it was observed that in plants, especially in Arabidopsis thaliana, increasing or generating the activity of a gene product mitochondrial with the activity of a “bifunctional protein (phosphoribosyltransferase and regulatory protein)”, preferably being encoded by a gene comprising the nucleic acid sequence SEQ ID NO. 106920, preferably the coding region thereof, conferred the production of or the increase in palmitic acid compared with the wild type control.

The nucleic acid sequence of Sll0374 from Synechocystis sp., e.g. as shown in the respective line in column 5 of Table I, application no. 11, has been published in C. And the activity of the gene product thereof is the activity of branched chain amino acid ABC transporter ATP-binding protein

Accordingly, in one embodiment, the process of the present invention for producing palmitic acid in a non-human organism, like a microorganism or a plant or a part thereof, comprises increasing or generating the activity of a gene product with the activity of a gene product conferring the activity of “branched chain amino acid ABC transporter ATP-binding protein”, especially from Synechocystis sp. or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said Sll0374, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said Sll0374, and preferably the activity is increased plastidic, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said Sll0374, or a functional equivalent or a homolog thereof as depicted in column 8 of Table II, application no. 11, preferably a homolog or functional equivalent as depicted in column 8 of Table II B, application no. 11, and being depicted in the same respective line as said Sll0374, and preferably the activity is increased plastidic.

Accordingly, in one embodiment, the molecule which activity is to be increased in the process of the invention is the gene product with an activity as a “branched chain amino acid ABC transporter ATP-binding protein”, preferably it is the molecule of section (a) or (b) of this paragraph.

In particular, it was observed that in plants, especially in Arabidopsis thaliana, increasing or generating the activity of a gene product plastidic with the activity of a “branched chain amino acid ABC transporter ATP-binding protein”, preferably being encoded by a gene comprising the nucleic acid sequence SEQ ID NO. 94741, preferably the coding region thereof, conferred the production of or the increase in palmitic acid compared with the wild type control.

The nucleic acid sequence of Sll1031 from Synechocystis sp., e.g. as shown in the respective line in column 5 of Table I, application no. 11, has been published in C. And the activity of the gene product thereof is the activity of carbon dioxide concentrating mechanism protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “carbon dioxide concentrating mechanism protein”, especially from Synechocystis sp. or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said Sll1031, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said Sll1031, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said Sll1031, or a functional equivalent or a homolog thereof as depicted in column 8 of Table II, application no. 11, preferably a homolog or functional equivalent as depicted in column 8 of Table II B, application no. 11, and being depicted in the same respective line as said Sll1031, and preferably the activity is increased mitochondrial.

Accordingly, in one embodiment, the molecule which activity is to be increased in the process of the invention is the gene product with an activity as a “carbon dioxide concentrating mechanism protein”, preferably it is the molecule of section (a) or (b) of this paragraph.

In particular, it was observed that in plants, especially in Arabidopsis thaliana, increasing or generating the activity of a gene product mitochondrial with the activity of a “carbon dioxide concentrating mechanism protein”, preferably being encoded by a gene comprising the nucleic acid sequence SEQ ID NO. 52634, preferably the coding region thereof, conferred the production of or the increase in palmitic acid compared with the wild type control.

The nucleic acid sequence of Sll1032 from Synechocystis sp., e.g. as shown in the respective line in column 5 of Table I, application no. 11, has been published in C. And the activity of the gene product thereof is the activity of sll1032-protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “sll1032-protein”, especially from Synechocystis sp. or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said Sll1032, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said Sll1032, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said Sll1032, or a functional equivalent or a homolog thereof as depicted in column 8 of Table II, application no. 11, preferably a homolog or functional equivalent as depicted in column 8 of Table II B, application no. 11, and being depicted in the same respective line as said Sll1032, and preferably the activity is increased mitochondrial.

Accordingly, in one embodiment, the molecule which activity is to be increased in the process of the invention is the gene product with an activity as a “sll1032-protein”, preferably it is the molecule of section (a) or (b) of this paragraph.

In particular, it was observed that in plants, especially in Arabidopsis thaliana, increasing or generating the activity of a gene product mitochondrial with the activity of a “sll1032-protein”, preferably being encoded by a gene comprising the nucleic acid sequence SEQ ID NO. 85723, preferably the coding region thereof, conferred the production of or the increase in palmitic acid compared with the wild type control.

The nucleic acid sequence of Sll1056 from Synechocystis sp., e.g. as shown in the respective line in column 5 of Table I, application no. 11, has been published in C. And the activity of the gene product thereof is the activity of phosphoribosylformyl glycinamidine synthase subunit.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “phosphoribosylformyl glycinamidine synthase subunit”, especially from Synechocystis sp. or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said Sll1056, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said Sll1056, and preferably the activity is increased plastidic, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said Sll1056, or a functional equivalent or a homolog thereof as depicted in column 8 of Table II, application no. 11, preferably a homolog or functional equivalent as depicted in column 8 of Table II B, application no. 11, and being depicted in the same respective line as said Sll1056, and preferably the activity is increased plastidic.

Accordingly, in one embodiment, the molecule which activity is to be increased in the process of the invention is the gene product with an activity as a “phosphoribosylformyl glycinamidine synthase subunit”, preferably it is the molecule of section (a) or (b) of this paragraph.

In particular, it was observed that in plants, especially in Arabidopsis thaliana, increasing or generating the activity of a gene product plastidic with the activity of a “phosphoribosylformyl glycinamidine synthase subunit”, preferably being encoded by a gene comprising the nucleic acid sequence SEQ ID NO. 52660, preferably the coding region thereof, conferred the production of or the increase in palmitic acid compared with the wild type control.

The nucleic acid sequence of Sll1450 from Synechocystis sp., e.g. as shown in the respective line in column 5 of Table I, application no. 11, has been published in C. And the activity of the gene product thereof is the activity of nitrate/nitrite transport protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “nitrate/nitrite transport protein”, especially from Synechocystis sp. or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said Sll1450, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said Sll1450, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said Sll1450, or a functional equivalent or a homolog thereof as depicted in column 8 of Table II, application no. 11, preferably a homolog or functional equivalent as depicted in column 8 of Table II B, application no. 11, and being depicted in the same respective line as said Sll1450, and preferably the activity is increased mitochondrial.

Accordingly, in one embodiment, the molecule which activity is to be increased in the process of the invention is the gene product with an activity as a “nitrate/nitrite transport protein”, preferably it is the molecule of section (a) or (b) of this paragraph.

In particular, it was observed that in plants, especially in Arabidopsis thaliana, increasing or generating the activity of a gene product mitochondrial with the activity of a “nitrate/nitrite transport protein”, preferably being encoded by a gene comprising the nucleic acid sequence SEQ ID NO. 54337, preferably the coding region thereof, conferred the production of or the increase in palmitic acid compared with the wild type control.

The nucleic acid sequence of Sll1498 from Synechocystis sp., e.g. as shown in the respective line in column 5 of Table I, application no. 11, has been published in C. And the activity of the gene product thereof is the activity of carbamoyl-phosphate synthase subunit.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “carbamoyl-phosphate synthase subunit”, especially from Synechocystis sp. or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said Sll1498, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said Sll1498, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said Sll1498, or a functional equivalent or a homolog thereof as depicted in column 8 of Table II, application no. 11, preferably a homolog or functional equivalent as depicted in column 8 of Table II B, application no. 11, and being depicted in the same respective line as said Sll1498, and preferably the activity is increased non-targeted.

Accordingly, in one embodiment, the molecule which activity is to be increased in the process of the invention is the gene product with an activity as a “carbamoyl-phosphate synthase subunit”, preferably it is the molecule of section (a) or (b) of this paragraph.

In particular, it was observed that in plants, especially in Arabidopsis thaliana, increasing or generating the activity of a gene product non-targeted with the activity of a “carbamoylphosphate synthase subunit”, preferably being encoded by a gene comprising the nucleic acid sequence SEQ ID NO. 95047, preferably the coding region thereof, conferred the production of or the increase in palmitic acid compared with the wild type control.

The nucleic acid sequence of Sll1545 from Synechocystis sp., e.g. as shown in the respective line in column 5 of Table I, application no. 11, has been published in C. And the activity of the gene product thereof is the activity of glutathione S-transferase.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “glutathione S-transferase”, especially from Synechocystis sp. or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said Sll1545, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said Sll1545, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said Sll1545, or a functional equivalent or a homolog thereof as depicted in column 8 of Table II, application no. 11, preferably a homolog or functional equivalent as depicted in column 8 of Table II B, application no. 11, and being depicted in the same respective line as said Sll1545, and preferably the activity is increased mitochondrial.

Accordingly, in one embodiment, the molecule which activity is to be increased in the process of the invention is the gene product with an activity as a “glutathione S-transferase”, preferably it is the molecule of section (a) or (b) of this paragraph.

In particular, it was observed that in plants, especially in Arabidopsis thaliana, increasing or generating the activity of a gene product mitochondrial with the activity of a “glutathione 5transferase”, preferably being encoded by a gene comprising the nucleic acid sequence SEQ ID NO. 11423, preferably the coding region thereof, conferred the production of or the increase in palmitic acid compared with the wild type control.

The nucleic acid sequence of Sll1598 from Synechocystis sp., e.g. as shown in the respective line in column 5 of Table I, application no. 11, has been published in C. And the activity of the gene product thereof is the activity of ABC metal ion transporter substrate-binding protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “ABC metal ion transporter substrate-binding protein”, especially from Synechocystis sp. or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said Sll1598, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said Sll1598, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said Sll1598, or a functional equivalent or a homolog thereof as depicted in column 8 of Table II, application no. 11, preferably a homolog or functional equivalent as depicted in column 8 of Table II B, application no. 11, and being depicted in the same respective line as said Sll1598, and preferably the activity is increased mitochondrial.

Accordingly, in one embodiment, the molecule which activity is to be increased in the process of the invention is the gene product with an activity as a “ABC metal ion transporter substratebinding protein”, preferably it is the molecule of section (a) or (b) of this paragraph.

In particular, it was observed that in plants, especially in Arabidopsis thaliana, increasing or generating the activity of a gene product mitochondrial with the activity of a “ABC metal ion transporter substrate-binding protein”, preferably being encoded by a gene comprising the nucleic acid sequence SEQ ID NO. 85743, preferably the coding region thereof, conferred the production of or the increase in palmitic acid compared with the wild type control.

The nucleic acid sequence of Sll1848 from Synechocystis sp., e.g. as shown in the respective line in column 5 of Table I, application no. 11, has been published in C. And the activity of the gene product thereof is the activity of acyl transferase.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “acyl transferase”, especially from Synechocystis sp. or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said Sll1848, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said Sll1848, and preferably the activity is increased plastidic, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said Sll1848, or a functional equivalent or a homolog thereof as depicted in column 8 of Table II, application no. 11, preferably a homolog or functional equivalent as depicted in column 8 of Table II B, application no. 11, and being depicted in the same respective line as said Sll1848, and preferably the activity is increased plastidic.

Accordingly, in one embodiment, the molecule which activity is to be increased in the process of the invention is the gene product with an activity as a “acyl transferase”, preferably it is the molecule of section (a) or (b) of this paragraph.

In particular, it was observed that in plants, especially in Arabidopsis thaliana, increasing or generating the activity of a gene product plastidic with the activity of a “acyl transferase”, preferably being encoded by a gene comprising the nucleic acid sequence SEQ ID NO. 86447, preferably the coding region thereof, conferred the production of or the increase in palmitic acid compared with the wild type control.

The nucleic acid sequence of Sll1917 from Synechocystis sp., e.g. as shown in the respective line in column 5 of Table I, application no. 11, has been published in C. And the activity of the gene product thereof is the activity of coproporphyrinogen III oxidase.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “coproporphyrinogen III oxidase”, especially from Synechocystis sp. or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said Sll1917, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said Sll1917, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said Sll1917, or a functional equivalent or a homolog thereof as depicted in column 8 of Table II, application no. 11, preferably a homolog or functional equivalent as depicted in column 8 of Table II B, application no. 11, and being depicted in the same respective line as said Sll1917, and preferably the activity is increased mitochondrial.

Accordingly, in one embodiment, the molecule which activity is to be increased in the process of the invention is the gene product with an activity as a “coproporphyrinogen III oxidase”, preferably it is the molecule of section (a) or (b) of this paragraph.

In particular, it was observed that in plants, especially in Arabidopsis thaliana, increasing or generating the activity of a gene product mitochondrial with the activity of a “coproporphyrinogen III oxidase”, preferably being encoded by a gene comprising the nucleic acid sequence SEQ ID NO. 11471, preferably the coding region thereof, conferred the production of or the increase in palmitic acid compared with the wild type control.

The nucleic acid sequence of Sll1920 from Synechocystis sp., e.g. as shown in the respective line in column 5 of Table I, application no. 11, has been published in C. And the activity of the gene product thereof is the activity of cation-transporting ATPase.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “cation-transporting ATPase”, especially from Synechocystis sp. or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said Sll1920, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said Sll1920, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said Sll1920, or a functional equivalent or a homolog thereof as depicted in column 8 of Table II, application no. 11, preferably a homolog or functional equivalent as depicted in column 8 of Table II B, application no. 11, and being depicted in the same respective line as said Sll1920, and preferably the activity is increased mitochondrial.

Accordingly, in one embodiment, the molecule which activity is to be increased in the process of the invention is the gene product with an activity as a “cation-transporting ATPase”, preferably it is the molecule of section (a) or (b) of this paragraph.

In particular, it was observed that in plants, especially in Arabidopsis thaliana, increasing or generating the activity of a gene product mitochondrial with the activity of a “cation-transporting ATPase”, preferably being encoded by a gene comprising the nucleic acid sequence SEQ ID NO. 55385, preferably the coding region thereof, conferred the production of or the increase in palmitic acid compared with the wild type control.

The nucleic acid sequence of Slr0949 from Synechocystis sp., e.g. as shown in the respective line in column 5 of Table I, application no. 11, has been published in C. And the activity of the gene product thereof is the activity of permease protein of ABC transporter.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “permease protein of ABC transporter”, especially from Synechocystis sp. or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said Slr0949, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said Slr0949, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said Slr0949, or a functional equivalent or a homolog thereof as depicted in column 8 of Table II, application no. 11, preferably a homolog or functional equivalent as depicted in column 8 of Table II B, application no. 11, and being depicted in the same respective line as said Slr0949, and preferably the activity is increased mitochondrial.

Accordingly, in one embodiment, the molecule which activity is to be increased in the process of the invention is the gene product with an activity as a “permease protein of ABC transporter”, preferably it is the molecule of section (a) or (b) of this paragraph.

In particular, it was observed that in plants, especially in Arabidopsis thaliana, increasing or generating the activity of a gene product mitochondrial with the activity of a “permease protein of ABC transporter”, preferably being encoded by a gene comprising the nucleic acid sequence SEQ ID NO. 119222, preferably the coding region thereof, conferred the production of or the increase in palmitic acid compared with the wild type control.

The nucleic acid sequence of Slr1099 from Synechocystis sp., e.g. as shown in the respective line in column 5 of Table I, application no. 11, has been published in C. And the activity of the gene product thereof is the activity of aromatic acid decarboxylase.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “aromatic acid decarboxylase”, especially from Synechocystis sp. or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said Slr1099, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said Slr1099, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said Slr1099, or a functional equivalent or a homolog thereof as depicted in column 8 of Table II, application no. 11, preferably a homolog or functional equivalent as depicted in column 8 of Table II B, application no. 11, and being depicted in the same respective line as said Slr1099, and preferably the activity is increased mitochondrial.

Accordingly, in one embodiment, the molecule which activity is to be increased in the process of the invention is the gene product with an activity as a “aromatic acid decarboxylase”, preferably it is the molecule of section (a) or (b) of this paragraph.

In particular, it was observed that in plants, especially in Arabidopsis thaliana, increasing or generating the activity of a gene product mitochondrial with the activity of a “aromatic acid decarboxylase”, preferably being encoded by a gene comprising the nucleic acid sequence SEQ ID NO. 116341, preferably the coding region thereof, conferred the production of or the increase in palmitic acid compared with the wild type control.

The nucleic acid sequence of Slr1250 from Synechocystis sp., e.g. as shown in the respective line in column 5 of Table I, application no. 11, has been published in C. And the activity of the gene product thereof is the activity of phosphate import ATP-binding protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “phosphate import ATP-binding protein”, especially from Synechocystis sp. or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said Slr1250, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said Slr1250, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said Slr1250, or a functional equivalent or a homolog thereof as depicted in column 8 of Table II, application no. 11, preferably a homolog or functional equivalent as depicted in column 8 of Table II B, application no. 11, and being depicted in the same respective line as said Slr1250, and preferably the activity is increased mitochondrial.

Accordingly, in one embodiment, the molecule which activity is to be increased in the process of the invention is the gene product with an activity as a “phosphate import ATP-binding protein”, preferably it is the molecule of section (a) or (b) of this paragraph.

In particular, it was observed that in plants, especially in Arabidopsis thaliana, increasing or generating the activity of a gene product mitochondrial with the activity of a “phosphate import ATP-binding protein”, preferably being encoded by a gene comprising the nucleic acid sequence SEQ ID NO. 116460, preferably the coding region thereof, conferred the production of or the increase in palmitic acid compared with the wild type control.

The nucleic acid sequence of Slr1312 from Synechocystis sp., e.g. as shown in the respective line in column 5 of Table I, application no. 11, has been published in C. And the activity of the gene product thereof is the activity of arginine decarboxylase.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “arginine decarboxylase”, especially from Synechocystis sp. or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said Slr1312, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said Slr1312, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said Slr1312, or a functional equivalent or a homolog thereof as depicted in column 8 of Table II, application no. 11, preferably a homolog or functional equivalent as depicted in column 8 of Table II B, application no. 11, and being depicted in the same respective line as said Slr1312, and preferably the activity is increased non-targeted.

Accordingly, in one embodiment, the molecule which activity is to be increased in the process of the invention is the gene product with an activity as a “arginine decarboxylase”, preferably it is the molecule of section (a) or (b) of this paragraph.

In particular, it was observed that in plants, especially in Arabidopsis thaliana, increasing or generating the activity of a gene product non-targeted with the activity of a “arginine decarboxylase”, preferably being encoded by a gene comprising the nucleic acid sequence SEQ ID NO. 58324, preferably the coding region thereof, conferred the production of or the increase in palmitic acid compared with the wild type control.

The nucleic acid sequence of Slr1791 from Synechocystis sp., e.g. as shown in the respective line in column 5 of Table I, application no. 11, has been published in C. And the activity of the gene product thereof is the activity of phosphoadenosine phosphosulfate reductase .

Accordingly, in one embodiment, the process of the present invention for producing palmitic acid in a non-human organism, like a microorganism or a plant or a part thereof, comprises increasing or generating the activity of a gene product with the activity of a gene product conferring the activity of “phosphoadenosine phosphosulfate reductase”, especially from Synechocystis sp. or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said Slr1791, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said Slr1791, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said Slr1791, or a functional equivalent or a homolog thereof as depicted in column 8 of Table II, application no. 11, preferably a homolog or functional equivalent as depicted in column 8 of Table II B, application no. 11, and being depicted in the same respective line as said Slr1791, and preferably the activity is increased mitochondrial.

Accordingly, in one embodiment, the molecule which activity is to be increased in the process of the invention is the gene product with an activity as a “phosphoadenosine phosphosulfate reductase”, preferably it is the molecule of section (a) or (b) of this paragraph.

In particular, it was observed that in plants, especially in Arabidopsis thaliana, increasing or generating the activity of a gene product mitochondrial with the activity of a “phosphoadenosine phosphosulfate reductase”, preferably being encoded by a gene comprising the nucleic acid sequence SEQ ID NO. 12140, preferably the coding region thereof, conferred the production of or the increase in palmitic acid compared with the wild type control.

The nucleic acid sequence of Slr2072 from Synechocystis sp., e.g. as shown in the respective line in column 5 of Table I, application no. 11, has been published in C. And the activity of the gene product thereof is the activity of threonine dehydratase.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “threonine dehydratase”, especially from Synechocystis sp. or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said Slr2072, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said Slr2072, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said Slr2072, or a functional equivalent or a homolog thereof as depicted in column 8 of Table II, application no. 11, preferably a homolog or functional equivalent as depicted in column 8 of Table II B, application no. 11, and being depicted in the same respective line as said Slr2072, and preferably the activity is increased mitochondrial.

Accordingly, in one embodiment, the molecule which activity is to be increased in the process of the invention is the gene product with an activity as a “threonine dehydratase”, preferably it is the molecule of section (a) or (b) of this paragraph.

In particular, it was observed that in plants, especially in Arabidopsis thaliana, increasing or generating the activity of a gene product mitochondrial with the activity of a “threonine dehydratase”, preferably being encoded by a gene comprising the nucleic acid sequence SEQ ID NO. 12341, preferably the coding region thereof, conferred the production of or the increase in palmitic acid compared with the wild type control.

The nucleic acid sequence of TTC0917 from Thermus thermophilus, e.g. as shown in the respective line in column 5 of Table I, application no. 11, has been published in E. And the activity of the gene product thereof is the activity of metal-dependent hydrolase.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “metal-dependent hydrolase”, especially from Thermus thermophilus or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said TTC0917, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said TTC0917, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said TTC0917, or a functional equivalent or a homolog thereof as depicted in column 8 of Table II, application no. 11, preferably a homolog or functional equivalent as depicted in column 8 of Table II B, application no. 11, and being depicted in the same respective line as said TTC0917, and preferably the activity is increased non-targeted.

Accordingly, in one embodiment, the molecule which activity is to be increased in the process of the invention is the gene product with an activity as a “metal-dependent hydrolase”, preferably it is the molecule of section (a) or (b) of this paragraph.

In particular, it was observed that in plants, especially in Arabidopsis thaliana, increasing or generating the activity of a gene product non-targeted with the activity of a “metal-dependent hydrolase”, preferably being encoded by a gene comprising the nucleic acid sequence SEQ ID NO. 61553, preferably the coding region thereof, conferred the production of or the increase in palmitic acid compared with the wild type control.

The nucleic acid sequence of TTC1550 from Thermus thermophilus, e.g. as shown in the respective line in column 5 of Table I, application no. 11, has been published in E. And the activity of the gene product thereof is the activity of homocitrate synthase.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “homocitrate synthase”, especially from Thermus thermophilus or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said TTC1550, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said TTC1550, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said TTC1550, or a functional equivalent or a homolog thereof as depicted in column 8 of Table II, application no. 11, preferably a homolog or functional equivalent as depicted in column 8 of Table II B, application no. 11, and being depicted in the same respective line as said TTC1550, and preferably the activity is increased non-targeted.

Accordingly, in one embodiment, the molecule which activity is to be increased in the process of the invention is the gene product with an activity as a “homocitrate synthase”, preferably it is the molecule of section (a) or (b) of this paragraph.

In particular, it was observed that in plants, especially in Arabidopsis thaliana, increasing or generating the activity of a gene product non-targeted with the activity of a “homocitrate synthase”, preferably being encoded by a gene comprising the nucleic acid sequence SEQ ID NO. 12974, preferably the coding region thereof, conferred the production of or the increase in palmitic acid compared with the wild type control.

The nucleic acid sequence of Ybr092c from Saccharomyces cerevisiae, e.g. as shown in the respective line in column 5 of Table I, application no. 11, has been published in A. And the activity of the gene product thereof is the activity of acid phosphatase precursor.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “acid phosphatase precursor”, especially from Saccharomyces cerevisiae or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said Ybr092c, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said Ybr092c, and preferably the activity is increased plastidic, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said Ybr092c, or a functional equivalent or a homolog thereof as depicted in column 8 of Table II, application no. 11, preferably a homolog or functional equivalent as depicted in column 8 of Table II B, application no. 11, and being depicted in the same respective line as said Ybr092c, and preferably the activity is increased plastidic.

Accordingly, in one embodiment, the molecule which activity is to be increased in the process of the invention is the gene product with an activity as a “acid phosphatase precursor”, preferably it is the molecule of section (a) or (b) of this paragraph.

In particular, it was observed that in plants, especially in Arabidopsis thaliana, increasing or generating the activity of a gene product plastidic with the activity of a “acid phosphatase precursor”, preferably being encoded by a gene comprising the nucleic acid sequence SEQ ID NO. 119347, preferably the coding region thereof, conferred the production of or the increase in palmitic acid compared with the wild type control.

The nucleic acid sequence of Ydl022w from Saccharomyces cerevisiae, e.g. as shown in the respective line in column 5 of Table I, application no. 11, has been published in A. And the activity of the gene product thereof is the activity of glycerol-3-phosphate dehydrogenase.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “glycerol-3-phosphate dehydrogenase”, especially from Saccharomyces cerevisiae or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said Ydl022w, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said Ydl022w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said Ydl022w, or a functional equivalent or a homolog thereof as depicted in column 8 of Table II, application no. 11, preferably a homolog or functional equivalent as depicted in column 8 of Table II B, application no. 11, and being depicted in the same respective line as said Ydl022w, and preferably the activity is increased non-targeted.

Accordingly, in one embodiment, the molecule which activity is to be increased in the process of the invention is the gene product with an activity as a “glycerol-3-phosphate dehydrogenase”, preferably it is the molecule of section (a) or (b) of this paragraph.

In particular, it was observed that in plants, especially in Arabidopsis thaliana, increasing or generating the activity of a gene product non-targeted with the activity of a “glycerol-3-phosphate dehydrogenase”, preferably being encoded by a gene comprising the nucleic acid sequence SEQ ID NO. 119408, preferably the coding region thereof, conferred the production of or the increase in palmitic acid compared with the wild type control.

The nucleic acid sequence of Ydl101c from Saccharomyces cerevisiae, e.g. as shown in the respective line in column 5 of Table I, application no. 11, has been published in A. And the activity of the gene product thereof is the activity of protein kinase.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “protein kinase”, especially from Saccharomyces cerevisiae or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said Ydl101c, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said Ydl101c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said Ydl101c, or a functional equivalent or a homolog thereof as depicted in column 8 of Table II, application no. 11, preferably a homolog or functional equivalent as depicted in column 8 of Table II B, application no. 11, and being depicted in the same respective line as said Ydl101c, and preferably the activity is increased non-targeted.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 122518, preferably the coding region thereof, conferred the production of or the increase in palmitic acid compared with the wild type control.

The nucleic acid sequence of Ydr131c from Saccharomyces cerevisiae, e.g. as shown in the respective line in column 5 of Table I, application no. 11, has been published in A. And the activity of the gene product thereof is the activity of F-box protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “F-box protein”, especially from Saccharomyces cerevisiae or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said Ydr131c, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said Ydr131c, and preferably the activity is increased plastidic, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said Ydr131c, or a functional equivalent or a homolog thereof as depicted in column 8 of Table II, application no. 11, preferably a homolog or functional equivalent as depicted in column 8 of Table II B, application no. 11, and being depicted in the same respective line as said Ydr131c, and preferably the activity is increased plastidic.

Accordingly, in one embodiment, the molecule which activity is to be increased in the process of the invention is the gene product with an activity as a “F-box protein”, preferably it is the molecule of section (a) or (b) of this paragraph.

In particular, it was observed that in plants, especially in Arabidopsis thaliana, increasing or generating the activity of a gene product plastidic with the activity of a “F-box protein”, preferably being encoded by a gene comprising the nucleic acid sequence SEQ ID NO. 14706, preferably the coding region thereof, conferred the production of or the increase in palmitic acid compared with the wild type control.

The nucleic acid sequence of Yjl073w from Saccharomyces cerevisiae, e.g. as shown in the respective line in column 5 of Table I, application no. 11, has been published in A. And the activity of the gene product thereof is the activity of DnaJ-like chaperone.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “DnaJ-like chaperone”, especially from Saccharomyces cerevisiae or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said Yjl073w, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said Yjl073w, and preferably the activity is increased plastidic, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said Yjl073w, or a functional equivalent or a homolog thereof as depicted in column 8 of Table II, application no. 11, preferably a homolog or functional equivalent as depicted in column 8 of Table II B, application no. 11, and being depicted in the same respective line as said Yjl073w, and preferably the activity is increased plastidic.

Accordingly, in one embodiment, the molecule which activity is to be increased in the process of the invention is the gene product with an activity as a “DnaJ-like chaperone”, preferably it is the molecule of section (a) or (b) of this paragraph.

In particular, it was observed that in plants, especially in Arabidopsis thaliana, increasing or generating the activity of a gene product plastidic with the activity of a “DnaJ-like chaperone”, preferably being encoded by a gene comprising the nucleic acid sequence SEQ ID NO. 64964, preferably the coding region thereof, conferred the production of or the increase in palmitic acid compared with the wild type control.

The nucleic acid sequence of Ylr304c from Saccharomyces cerevisiae, e.g. as shown in the respective line in column 5 of Table I, application no. 11, has been published in A. And the activity of the gene product thereof is the activity of aconitate hydratase.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “aconitate hydratase”, especially from Saccharomyces cerevisiae or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said Ylr304c, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said Ylr304c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said Ylr304c, or a functional equivalent or a homolog thereof as depicted in column 8 of Table II, application no. 11, preferably a homolog or functional equivalent as depicted in column 8 of Table II B, application no. 11, and being depicted in the same respective line as said Ylr304c, and preferably the activity is increased non-targeted.

Accordingly, in one embodiment, the molecule which activity is to be increased in the process of the invention is the gene product with an activity as a “aconitate hydratase”, preferably it is the molecule of section (a) or (b) of this paragraph.

In particular, it was observed that in plants, especially in Arabidopsis thaliana, increasing or generating the activity of a gene product non-targeted with the activity of a “aconitate hydratase”, preferably being encoded by a gene comprising the nucleic acid sequence SEQ ID NO. 66772, preferably the coding region thereof, conferred the production of or the increase in palmitic acid compared with the wild type control.

The nucleic acid sequence of Ymr013c from Saccharomyces cerevisiae, e.g. as shown in the respective line in column 5 of Table I, application no. 11, has been published in A. And the activity of the gene product thereof is the activity of ymr013c-protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “ymr013c-protein”, especially from Saccharomyces cerevisiae or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said Ymr013c, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said Ymr013c, and preferably the activity is increased plastidic, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said Ymr013c, or a functional equivalent or a homolog thereof as depicted in column 8 of Table II, application no. 11, preferably a homolog or functional equivalent as depicted in column 8 of Table II B, application no. 11, and being depicted in the same respective line as said Ymr013c, and preferably the activity is increased plastidic.

Accordingly, in one embodiment, the molecule which activity is to be increased in the process of the invention is the gene product with an activity as a “ymr013c-protein”, preferably it is the molecule of section (a) or (b) of this paragraph.

In particular, it was observed that in plants, especially in Arabidopsis thaliana, increasing or generating the activity of a gene product plastidic with the activity of a “ymr013c-protein”, preferably being encoded by a gene comprising the nucleic acid sequence SEQ ID NO. 91958, preferably the coding region thereof, conferred the production of or the increase in palmitic acid compared with the wild type control.

The nucleic acid sequence of Ypl271w from Saccharomyces cerevisiae, e.g. as shown in the respective line in column 5 of Table I, application no. 11, has been published in A. And the activity of the gene product thereof is the activity of ATPase epsilon subunit.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “ATPase epsilon subunit”, especially from Saccharomyces cerevisiae or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said Ypl271w, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said Ypl271w, and preferably the activity is increased plastidic, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said Ypl271w, or a functional equivalent or a homolog thereof as depicted in column 8 of Table II, application no. 11, preferably a homolog or functional equivalent as depicted in column 8 of Table II B, application no. 11, and being depicted in the same respective line as said Ypl271w, and preferably the activity is increased plastidic.

Accordingly, in one embodiment, the molecule which activity is to be increased in the process of the invention is the gene product with an activity as a “ATPase epsilon subunit”, preferably it is the molecule of section (a) or (b) of this paragraph.

In particular, it was observed that in plants, especially in Arabidopsis thaliana, increasing or generating the activity of a gene product plastidic with the activity of a “ATPase epsilon subunit”, preferably being encoded by a gene comprising the nucleic acid sequence SEQ ID NO. 118212, preferably the coding region thereof, conferred the production of or the increase in palmitic acid compared with the wild type control.

The nucleic acid sequence of 49747384_SOYBEAN from Glycine max, e.g. as shown in the respective line in column 5 of Table I, application no. 11, is unpublished. And the activity of the gene product thereof is the activity of 49747384_SOYBEAN-protein.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “49747384_SOYBEAN-protein”, especially from Glycine max or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said 49747384_SOYBEAN, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said 49747384_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said 49747384_SOYBEAN, or a functional equivalent or a homolog thereof as depicted in column 8 of Table II, application no. 11, preferably a homolog or functional equivalent as depicted in column 8 of Table II B, application no. 11, and being depicted in the same respective line as said 49747384_SOYBEAN, and preferably the activity is increased non-targeted.

Accordingly, in one embodiment, the molecule which activity is to be increased in the process of the invention is the gene product with an activity as a “49747384_SOYBEAN-protein”, preferably it is the molecule of section (a) or (b) of this paragraph.

In particular, it was observed that in plants, especially in Arabidopsis thaliana, increasing or generating the activity of a gene product non-targeted with the activity of a “49747384_SOYBEAN-protein”, preferably being encoded by a gene comprising the nucleic acid sequence SEQ ID NO. 69, preferably the coding region thereof, conferred the production of or the increase in palmitic acid compared with the wild type control.

The nucleic acid sequence of 59582753_SOYBEAN from Glycine max, e.g. as shown in the respective line in column 5 of Table I, application no. 11, is unpublished. And the activity of the gene product thereof is the activity of glucose-6-phosphate 1-dehydrogenase.

Accordingly, in one embodiment, the process of the present invention for producing palmitic acid in a non-human organism, like a microorganism or a plant or a part thereof, comprises increasing or generating the activity of a gene product with the activity of a gene product conferring the activity of “glucose-6-phosphate 1-dehydrogenase”, especially from Glycine max or its functional equivalent or its homolog, e.g. the increase of
(a) a gene product of a gene comprising the nucleic acid molecule as shown in the respective line in column 5 of Table I, application no. 11, preferably the coding region thereof, or a homolog or a fragment thereof, and being depicted in the same respective line as said 59582753_SOYBEAN, or a functional equivalent or a homolog thereof as shown in column 8 of Table I, application no. 11, preferably the coding region thereof, particularly a homolog or functional equivalent as shown in column 8 of Table I B, application no. 11, and being depicted in the same respective line as said 59582753_SOYBEAN, and preferably the activity is increased plastidic, or
(b) a polypeptide comprising a polypeptide, a consensus sequence or at least a polypeptide motif as shown in the respective line in column 5 of Table II or in column 8 of Table IV, application no. 11, respectively, and being depicted in the same respective line as said 59582753_SOYBEAN, or a functional equivalent or a homolog thereof as depicted in column 8 of Table II, application no. 11, preferably a homolog or functional equivalent as depicted in column 8 of Table II B, application no. 11, and being depicted in the same respective line as said 59582753_SOYBEAN, and preferably the activity is increased plastidic.

Accordingly, in one embodiment, the molecule which activity is to be increased in the process of the invention is the gene product with an activity as a “glucose-6-phosphate 1-dehydrogenase”, preferably it is the molecule of section (a) or (b) of this paragraph.

In particular, it was observed that in plants, especially in Arabidopsis thaliana, increasing or generating the activity of a gene product plastidic with the activity of a “glucose-6-phosphate 1dehydrogenase”, preferably being encoded by a gene comprising the nucleic acid sequence SEQ ID NO. 16883, preferably the coding region thereof, conferred the production of or the increase in palmitic acid compared with the wild type control.

[0105.1.8.11] to [0107.1.8.11] for the disclosure of these paragraphs see [0105.1.8.8] to [0107.1.8.8] above.

A protein having an activity conferring an increase in the amount or level of the fine chemical palmitic acid, upon targeting to the plastids or mitochondria or upon non-targeting, preferably has the structure of the respective polypeptide described herein, in particular of the polypeptides comprising the consensus sequence or at least one polypeptide motifs as shown in the respective line in Table IV, application no. 11, column 8 or of the polypeptide comprising an amino acid sequence as disclosed in the respective line in Table II, application no. 11, columns 5 or 8, or homologs or fragments thereof as described herein, or is encoded by the nucleic acid molecule characterized herein or the nucleic acid molecule according to the invention, for example by the nucleic acid molecule as shown in the respective line in Table I, application no. 11, columns 5 or 8, preferably the coding region thereof, or homologs or fragments thereof and has the herein mentioned activity.

[0109.1.8.11] to [0110.1.8.11] for the disclosure of these paragraphs see [0109.1.8.8] to [0110.1.8.8] above.

In one embodiment, the process of the present invention comprises one or more of the following steps
(a) stabilizing a protein conferring the generation or increased expression of a protein encoded by the nucleic acid molecule of the invention or of the polypeptide of the invention, e.g. of a polypeptide having the activity selected from the group consisting of 2-C-methyl-D-erythritol 4-phosphate cytidylyltransferase, 3-ketoacyl-CoA thiolase, 49747384_SOYBEAN-protein, AAS bifunctional protein, ABC metal ion transporter substrate-binding protein, acid phosphatase precursor, acid shock protein, aconitate hydratase, acyl transferase, arginine decarboxylase, aromatic acid decarboxylase, At1g19800-protein, At1g23110-protein, At2g45420-protein, ATPase epsilon subunit, b0050-protein, b1234-protein, b1248-protein, b1330-protein, b1670-protein, b1837-protein, b2032-protein, b2099-protein, b2107-protein, b2399-protein, b2682-protein, b2849-protein, b3814-protein, bifunctional protein (phosphoribosyltransferase and regulatory protein), branched chain amino acid ABC transporter ATP-binding protein, calcium-dependent protein kinase, carbamoyl-phosphate synthase subunit, carbon dioxide concentrating mechanism protein, cation-transporting ATPase, CCAAT-binding transcription factor, chlorophyllase, colanic acid biosynthesis protein, coproporphyrinogen III oxidase, DnaJ-like chaperone, ethanolamine utilization protein, F-box protein, Fe—S subunit of oxidoreductase, flavodoxin, glucose-6-phosphate 1-dehydrogenase, glutaredoxin, glutathione S-transferase, glycerol-3-phosphate dehydrogenase, heat shock transcription factor, HesB/YadR/YfhF family protein, homocitrate synthase, lipoprotein precursor, metal-dependent hydrolase, monothiol glutaredoxin, NADH-quinone oxidoreductase subunit, nitrate/nitrite transport protein, oxidoreductase, peptidyl-prolyl cis-trans isomerase, permease protein of ABC transporter, phosphate import ATP-binding protein, phosphoadenosine phosphosulfate reductase , phosphoribosylformyl glycinamidine synthase subunit, protein kinase, protein phosphatase, purine nucleoside phosphorylase, recombinase A, sll1032-protein, sodium/proton antiporter, ThiF family protein, threonine dehydratase, threonine efflux protein, transcription factor, and ymr013c-protein, or of a polypeptide as indicated in the respective line in Table II, application no. 11, columns 5 or 8, or its homologs or fragments, and conferring the production of or an increase in palmitic acid, respectively; and/or
(b) stabilizing a mRNA conferring the generation or increased expression of a FCRP, e.g. protein encoded by the nucleic acid molecule of the invention or its homologs or fragments, or of a mRNA encoding the polypeptide of the present invention having the hereinmentioned activity selected from the group consisting of said activities mentioned in (a) and conferring the production of or an increase in palmitic acid, respectively; and/or
(c) increasing the specific activity of a protein conferring the increased expression of a FCRP, e.g. a protein encoded by the nucleic acid molecule of the invention or of the polypeptide of the present invention having herein-mentioned palmitic acid generating or increasing activity, respectively, e.g. of a polypeptide having the activity of a protein as indicated in the respective line in Table II, application no. 11, columns 5 or 8, or its homologs or fragments, or decreasing the inhibitory regulation of the polypeptide of the invention; and/or
(d) generating or increasing the expression of an endogenous or artificial transcription factor mediating the expression of a protein conferring the generation or increased expression of a FCRP, e.g. a protein encoded by the nucleic acid molecule of the invention or of the polypeptide of the invention having herein-mentioned activity selected from the group consisting of said activities mentioned in (a) and conferring a palmitic acid increasing activity, respectively, e.g. of a polypeptide having the activity of a protein as indicated in the respective line in Table II, application no. 11, columns 5 or 8 or its homologs; and/or
(e) stimulating activity of a protein conferring the increased expression of a FCRP, e.g. a protein encoded by the nucleic acid molecule of the present invention or a polypeptide of the present invention having herein-mentioned activity selected from the group consisting of said activities mentioned in (a) and conferring a palmitic acid increasing activity, respectively, e.g. of a polypeptide having the activity of a protein as indicated in the respective line in Table II, application no. 11, columns 5 or 8, or its homologs or fragments, by adding one or more exogenous inducing factors to the non-human organism or parts thereof; and/or
(f) expressing a transgenic gene encoding a protein conferring the increased expression of a FCRP, e.g. a polypeptide encoded by the nucleic acid molecule of the present invention or a polypeptide of the present invention, having herein-mentioned activity selected from the group consisting of said activities mentioned in (a) and conferring a palmitic acid increasing activity, respectively, e.g. of a polypeptide having the activity of a protein as indicated in the respective line in Table II, application no. 11, columns 5 or 8, or its homologs or fragments, and/or
(g) increasing the copy number of a gene conferring the increased expression of a nucleic acid molecule encoding a FCRP, e.g. a polypeptide encoded by the nucleic acid molecule of the invention or the polypeptide of the invention having herein-mentioned activity selected from the group consisting of said activities mentioned in (a) and conferring a palmitic acid increasing activity, respectively, e.g. of a polypeptide having the activity of a protein as indicated in the respective line in Table II, application no. 11, columns 5 or 8, or its homologs or fragments; and/or
(h) increasing the expression of the endogenous gene encoding the FCRP, polypeptide of the invention, e.g. a polypeptide encoded by the nucleic acid molecule of the invention or the polypeptide of the invention having herein-mentioned activity selected from the group consisting of said activities mentioned in (a) and conferring a palmitic acid; increasing activity, respectively, e.g. of a polypeptide having the activity of a protein as indicated in the respective line in Table II, application no. 11, columns 5 or 8, or its homologs or fragments, by adding positive expression or removing negative expression elements, e.g. homologous recombination can be used to either introduce positive regulatory elements like for plants the 35S enhancer into the promoter or to remove repressor elements form regulatory regions. Further gene conversion methods can be used to disrupt repressor elements or to enhance to activity of positive elements. Positive elements can be randomly introduced in plants by T-DNA or transposon mutagenesis and lines can be identified in which the positive elements have be integrated near to a gene of the invention, the expression of which is thereby enhanced; and/or
(i) modulating growth conditions of the non-human organism in such a manner, that the expression or activity of the gene encoding the FCRP, e.g. a protein of the invention or the protein itself is enhanced, for example microorganisms or plants can be grown for example under a higher temperature regime leading to an enhanced expression of heat shock proteins, which can lead to an enhanced palmitic acid production, respectively; and/or
(j) selecting of non-human organisms with especially high activity of the proteins of the invention from natural or from mutagenized resources and breeding them into the target organisms, e.g. the elite crops; and/or
(k) directing a protein encoded by the nucleic acid molecule of the invention or of the polypeptide of the present invention having herein-mentioned activity selected from the group consisting of said activities mentioned in (a) and conferring a palmitic acid increasing activity, respectively, e.g. of a polypeptide having the activity of a protein as indicated in the respective line in Table II, application no. 11, columns 5 or 8, or its homologs or fragments, if for the corresponding nucleic acid molecule in the respective line in column 6 of Table I the term “plastidic” is indicated, to the plastids by the addition of a plastidial targeting sequence or if for the corresponding nucleic acid molecule in the respective line in column 6 of Table I the term “mitochondrial” is indicated, to the mitochondria by the addition of a mitochondrial targeting sequence; and/or
(l) generating the expression of a protein encoded by the nucleic acid molecule of the invention or of the polypeptide of the present invention having herein-mentioned activity selected from the group consisting of said activities mentioned in (a) and conferring a palmitic acid increasing activity, respectively, e.g. of a polypeptide having the activity of a protein as indicated in the respective line in Table II, application no. 11, columns 5 or 8, or its homologs or fragments, if for the corresponding nucleic acid molecule in the respective line in column 6 of Table I the term “plastidic” or “mitochondrial” is indicated, in these organelles by the stable or transient transformation, advantageously stable transformation, of organelles, preferably plastids or mitochondria, with an inventive nucleic acid sequence preferably in form of an expression cassette containing said sequence leading to the expression of the nucleic acids or polypeptides of the invention in the respective organelle; and/or
(m) generating the expression of a protein encoded by the nucleic acid molecule of the invention or of the polypeptide of the present invention having herein-mentioned activity selected from the group consisting of said activities mentioned in (a) and conferring a palmitic acid increasing activity, respectively, e.g. of a polypeptide having the activity of a protein as indicated in the respective line in Table II, application no. 11, columns 5 or 8, or its homologs or fragments, if for the corresponding nucleic acid molecule in the respective line in column 6 of Table I the term “plastidic” or “mitochondrial” is indicated, in these organelles by integration of a nucleic acid of the invention into the genome of the respective organelle under control of preferable a promoter selective for the respective organelle.

Preferably, said mRNA is the coding region of the nucleic acid molecule of the present invention and/or the protein conferring the increased expression of a protein encoded by the coding region of the nucleic acid molecule of the present invention alone or linked to a transit nucleic acid sequence or transit peptide encoding nucleic acid sequence or the polypeptide having the herein mentioned activity, e.g. conferring the generation of or increase of palmitic acid, respectively, after increasing the expression or activity of the encoded polypeptide, non-targeted or in organelles such as plastids and/or mitochondria, preferably plastids, or having the activity of a polypeptide having an activity as the protein as shown in the respective line in Table II, application no. 11, column 3, or its homologs. Preferably the increase of palmitic acid, respectively, takes place non-targeted or in plastids and/or mitochondria, preferably non-targeted or in plastids.

[0113.1.8.11] to [0122.1.8.11] for the disclosure of these paragraphs see [0113.1.8.8] to [0122.1.8.8] above.

The activation of an endogenous polypeptide having above-mentioned activity, e.g. having the activity of a protein as shown in the respective line in Table II, application no. 11, column 3 or of the polypeptide of the invention, e.g. conferring the increase of the fine chemical palmitic acid, respectively, by increase of expression or activity in the cytoplasm, and/or in the cytosol, and/or in an organelle, such as plastids or mitochondria, can also be increased by introducing a synthetic transcription factor, which binds close to the coding region of the gene encoding the protein as shown in the respective line in Table II, application no. 11, column 5 or 8, or homologs or fragments thereof, and activates its transcription. A chimeric zinc finger protein can be constructed, which comprises a specific DNA-binding domain and an activation domain as e.g. the VP16 domain of Herpes Simplex virus. The specific binding domain can bind to the regulatory region of the gene encoding the protein as shown in the respective line in Table II, application no. 11, column 5 or 8, or homologs or fragments thereof. The expression of the chimeric transcription factor in a non-human organism, in particular in a plant, leads to a specific expression of the protein as shown in the respective line in Table II, application no. 11, column 5 or 8, or homologs or fragments thereof, see e.g. in WO01/52620, Oriz, Proc. Natl. Acad. Sci. USA 99, 13290 (2002), or Guan, Proc. Natl. Acad. Sci. USA 99, 13296 (2002).

[0124.1.8.11] to [0127.1.8.11] for the disclosure of these paragraphs see [0124.1.8.8] to [0127.1.8.8] above.

Owing to the introduction of a gene or a plurality of genes conferring the expression of the nucleic acid molecule of the invention or the polypeptide of the invention, for example the nucleic acid construct mentioned below, or encoding the protein as shown in the respective line in Table II, application no. 11, column 5 or 8, or homologs or fragments thereof, into a non-human organism alone or in combination with other genes, it is possible not only to increase the biosynthetic flux towards the end product, but also to increase, modify or create de novo an advantageous, preferably novel metabolites composition in the non-human organism, e.g. an advantageous composition comprising a higher content of (from a viewpoint of nutritional physiology limited) palmitic acid and if desired other fatty acids, and/or other metabolites, in free or bound form.

for the disclosure of this paragraph see [0129.1.8.8] above.

Accordingly, in one embodiment, the process according to the invention relates to a process, which comprises:
(a) providing a non-human organism, preferably a microorganism, a plant cell, a plant tissue, a plant or a part thereof, more preferably a microorganism, a plant tissue, a plant or a part thereof;
(b) increasing the activity of a protein as shown in the respective line in Table II, application no. 11, column 3 or of a polypeptide being encoded by the respective nucleic acid molecule of the present invention and described below, e.g. conferring an increase of the fine chemical palmitic acid, respectively, in the non-human organism, preferably in the microorganism, the plant cell, the plant tissue, the plant or a part thereof, more preferably a microorganism, a plant tissue, a plant or a part thereof, especially cytoplasmic or in an organelle, like plastids or mitochondria,
(c) growing the non-human organism, preferably the microorganism, the plant cell, the plant tissue, the plant or a part thereof, under conditions which permit the production of said fine chemical in the non-human organism, preferably the microorganism, the plant cell, the plant tissue, the plant or a part thereof; and
(d) if desired, recovering, optionally isolating, said fine chemical, in free and/or bound form, and, optionally further free and/or bound fatty acids, and/or other metabolites synthesized by the non-human organism, the microorganism, the plant cell, the plant tissue, the plant or a part thereof.

[0131.1.8.11] to [0139.1.8.11] for the disclosure of these paragraphs see [0131.1.8.8] to [0139.1.8.8] above.

In another embodiment, the invention relates to a (isolated) nucleic acid molecule, which comprises a nucleic acid molecule selected from the group consisting of:
(a) a nucleic acid molecule encoding a polypeptide shown in Table II, application no. 11, column 5 or 8, preferably shown in Table II B, application no. 11, column 8, or a homolog or a fragment thereof;
(b) a nucleic acid molecule shown in Table I, application no. 11, column 5 or 8, preferably shown in Table I B, application no. 11, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 11, preferably in column 8 of Table II B, application no. 11;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 11, preferably shown in column 8 of Table I B, application no. 11, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 11;
(i) a nucleic acid molecule encoding a polypeptide having the activity represented by a respective protein as depicted in column 5 of Table II, application no. 11;
(j) a nucleic acid molecule comprising a polynucleotide which is obtained by amplifying a cDNA library or a genomic library using the primers in Table III, application no. 11, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 11.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II application no. 11 and confers a generation or an increase of the respective fine chemical in a non-human organism or a part thereof as compared to a corresponding wild-type non-human organism or part thereof.

In another embodiment, the invention relates to a (isolated) nucleic acid molecule, which comprises a nucleic acid molecule selected from the group consisting of:
(a) a nucleic acid molecule encoding a polypeptide shown in Table II, column 5 or 8, application no. 11, preferably shown in Table II A, application no. 11, in column 5 or in Table II A, application no. 11, column 8 or in Table II B, application no. 11, column 8, or a homolog or a fragment thereof;
(b) a nucleic acid molecule shown in Table I, application no. 11, column 5 or 8, preferably shown in Table I A, application no. 11, in column 5 or in Table I A, application no. 11, column 8 or in Table I B, application no. 11, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 11, preferably shown in Table II A, application no. 11, in column 5 or in Table II A, application no. 11, column 8 or in Table II B, application no. 11, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 11, preferably shown in Table I A, application no. 11, in column 5 or in Table I A, application no. 11, column 8 or in Table I B column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 11;
(i) a nucleic acid molecule encoding a polypeptide having the activity represented by a respective protein as depicted in column 5 of Table II, application no. 11;
(j) a nucleic acid molecule comprising a polynucleotide which is obtained by amplifying a cDNA library or a genomic library using the primers in Table III, application no. 11, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt,500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto;
   whereby, preferably, the nucleic acid molecule according to (a), (b), (c), (d), (e), (f), (g), (h), (i), (j) or (k) distinguishes over the sequence depicted in Table I A and/or I B, application no. 11, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 11, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 11, column 5 or 8, or the coding region thereof, respectively. In a further embodiment the nucleic acid molecule does not encode the polypeptide sequence shown in Table II A and/or II B, application no. 11, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 11, column 5 or 8 and does not encode a protein of the sequence shown in Table II A and/or II B, application no. 11, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 11, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 11, column 5 or 8 but less than 100%, preferably less than 99.999%, 99.99%, 99.9%, 99%, 98%, 97%, 96% or 95% identical to the sequence shown in Table II A and/or II B, application no. 11, column 5 or 8.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 11.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II, application no. 11, and confers a generation or an increase of the respective fine chemical in a non-human organism or a part thereof as compared to a corresponding wild-type non-human organism or part thereof.

[0144.1.8.11] to [0155.1.8.11] for the disclosure of these paragraphs see [0144.1.8.8] to [0155.1.8.8] above.

In a preferred embodiment a nucleic acid construct, for example an expression cassette, comprises upstream, i.e. at the 5′ end of the encoding sequence, a promoter and downstream, i.e. at the 3′ end, a polyadenylation signal and optionally other regulatory elements which are operably linked to the intervening encoding sequence with one of the nucleic acids as depicted in the respective line in Table I, application no. 11, column 5 or 8, preferably the coding region thereof, or a homolog or a fragment thereof. By an operable linkage is meant the sequential arrangement of promoter, encoding sequence, terminator, optionally other regulatory elements and optionally targeting sequences in such a way that each of the regulatory elements/targeting sequence can fulfill its function in the expression of the encoding sequence in due manner. In one embodiment the sequences preferred for operable linkage are targeting sequences for ensuring subcellular localization in organelles, like plastids or mitochondria. However, targeting sequences for ensuring subcellular localization in the endoplasmic reticulum (=ER), in the nucleus, in oil corpuscles or other compartments may also be employed as well as translation promoters such as the 5′ lead sequence in tobacco mosaic virus (Gallie et al., Nucl. Acids Res. 15, 8693 (1987)).

[0157.1.8.11] for the disclosure of this paragraph see [0157.1.8.8] above.

In an embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 11, column 5 or 8, preferably shown in Table II A, application no. 11, column 5, or in Table II A, application no. 11, column 8, or in Table II B, application no. 11, column 8, or a homolog or a fragment thereof;
   (b) a nucleic acid molecule shown in Table I, application no. 11, column 5 or 8, preferably shown in Table I A, application no. 11, column 5, or in Table I A, application no. 11, column 8, or in Table I B, application no. 11, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 11, preferably shown in Table II A, application no. 11, column 5, or in Table II A, application no. 11, column 8, or in Table II B, application no. 11, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 11, preferably shown in Table I A, application no. 11, column 5, or in Table I A, application no. 11, column 8, or in Table I B, application no. 11, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 11;
   (i) a nucleic acid molecule encoding a polypeptide having the activity represented by a respective protein as depicted in column 5 of Table II, application no. 11;
   (j) a nucleic acid molecule comprising a polynucleotide which is obtained by amplifying a cDNA library or a genomic library using the primers in Table III, application no. 11, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto. and, optionally
3) nucleic acid molecule encoding a transit peptide, preferably encoding a plastidal transit peptide or a mitochondrial transit peptide;
   which are operable linked.
   (All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 11.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 11 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 11, in the same line as of the respective nucleic acid molecule according to 2) “plastidic” is depicted, or encoding a mitochondrial transit peptide, especially in case in column 6 of Table I, application no. 11, in the same line as of the respective nucleic acid molecule according to 2) “mitochondrial” is depicted.

In another embodiment thereof the expression cassette does not comprise 3) a nucleic acid molecule encoding a transit peptide, especially in case in column 6 of Table I, application no. 11, in the same line as of the respective nucleic acid molecule according to 2) “non-targeted” is depicted.

In a further embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 11, column 5 or 8, preferably shown in Table II A, application no. 11, column 5, or in Table II A, application no. 11, column 8, or in Table II B, application no. 11, column 8, or a homolog or a fragment thereof;
   (b) a nucleic acid molecule shown in Table I, application no. 11, column 5 or 8, preferably shown in Table I A, application no. 11, column 5, or in Table I A, application no. 11, column 8, or in Table I B, application no. 11, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 11, preferably shown in Table II A, application no. 11, column 5, or in Table II A, application no. 11, column 8, or in Table II B, application no. 11, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 11, preferably shown in Table I A, application no. 11, column 5, or in Table I A, application no. 11, column 8,or in Table I B, application no. 11, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 11;
   (i) a nucleic acid molecule encoding a polypeptide having the activity represented by a respective protein as depicted in column 5 of Table II, application no. 11;
   (j) a nucleic acid molecule comprising a polynucleotide which is obtained by amplifying a cDNA library or a genomic library using the primers in Table III, application no. 11, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, optionally
3) nucleic acid molecule encoding a transit peptide, preferably encoding a plastidal transit peptide or a mitochondrial transit peptide;
   which are operable linked and
   whereby, preferably, the nucleic acid molecule according to 2 (a), (b), (c), (d), (e), (f), (g), (h), (i), (j) or (k) distinguishes over the sequence depicted in Table I A and/or I B, application no. 11, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention according to 2) does not consist of the sequence shown in Table I A and/or I B, application no. 11, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 11, column 5 or 8, or the coding region thereof, respectively. In a further embodiment the nucleic acid molecule does not encode the polypeptide sequence shown in Table II A and/or II B, application no. 11, column 5 or 8 but homologs thereof.

Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 11, column 5 or 8 and does not encode a protein of the sequence shown in Table II A and/or II B, application no. 11, column 5 or 8. Accordingly, in one embodiment, the protein encoded by a sequence of a nucleic acid according to 2(a), (b), (c), (d), (e), (f), (g), (h), (i), (j) or (k) does not consist of the sequence shown in Table I A and/or I B, application no. 11, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 11, column 5 or 8 but less than 100%, 99.999%, 99.99%, 99.9%, 99%, 98%, 97%, 96% or 95% identical to the sequence shown in Table II A and/or II B, application no. 11, column 5 or 8.

(All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 11.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 11 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 11, in the same line as of the respective nucleic acid molecule according to 2) “plastidic” is depicted, or encoding a mitochondrial transit peptide, especially in case in column 6 of Table I, application no. 11, in the same line as of the respective nucleic acid molecule according to 2) “mitochondrial” is depicted.

In another embodiment thereof the expression cassette does not comprise 3) a nucleic acid molecule encoding a transit peptide, especially in case in column 6 of Table I, application no. 11, in the same line as of the respective nucleic acid molecule according to 2) “non-targeted” is depicted.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17451, or a homolog or fragment thereof, or a homolog thereof being depicted in Table I, application no. 11, column 8, in the same line as SEQ ID NO. 17451, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 17451 is depicted, plastidic is mentioned, or a mitochondrial transit peptide in case in Table I, application no. 11, in column 6 in the same line as SEQ ID NO. 17451 is depicted, mitochondrial is mentioned; which is operable linked, too.

In another embodiment thereof the expression cassette does not comprise an additional transit peptide in case in Table I, application no. 11, in column 6 in the same line as SEQ ID NO. 17451 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 113651, or a homolog or fragment thereof, or a homolog thereof being depicted in Table I, application no. 11, column 8, in the same line as SEQ ID NO. 113651, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 113651 is depicted, plastidic is mentioned, or a mitochondrial transit peptide in case in Table I, application no. 11, in column 6 in the same line as SEQ ID NO. 113651 is depicted, mitochondrial is mentioned; which is operable linked, too.

In another embodiment thereof the expression cassette does not comprise an additional transit peptide in case in Table I, application no. 11, in column 6 in the same line as SEQ ID NO. 113651 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17637, or a homolog or fragment thereof, or a homolog thereof being depicted in Table I, application no. 11, column 8, in the same line as SEQ ID NO. 17637, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 17637 is depicted, plastidic is mentioned, or a mitochondrial transit peptide in case in Table I, application no. 11, in column 6 in the same line as SEQ ID NO. 17637 is depicted, mitochondrial is mentioned; which is operable linked, too.

In another embodiment thereof the expression cassette does not comprise an additional transit peptide in case in Table I, application no. 11, in column 6 in the same line as SEQ ID NO. 17637 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 102086, or a homolog or fragment thereof, or a homolog thereof being depicted in Table I, application no. 11, column 8, in the same line as SEQ ID NO. 102086, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 102086 is depicted, plastidic is mentioned, or a mitochondrial transit peptide in case in Table I, application no. 11, in column 6 in the same line as SEQ ID NO. 102086 is depicted, mitochondrial is mentioned; which is operable linked, too.

In another embodiment thereof the expression cassette does not comprise an additional transit peptide in case in Table I, application no. 11, in column 6 in the same line as SEQ ID NO. 102086 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 1061, or a homolog or fragment thereof, or a homolog thereof being depicted in Table I, application no. 11, column 8, in the same line as SEQ ID NO. 1061, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 1061 is depicted, plastidic is mentioned, or a mitochondrial transit peptide in case in Table I, application no. 11, in column 6 in the same line as SEQ ID NO. 1061 is depicted, mitochondrial is mentioned; which is operable linked, too.

In another embodiment thereof the expression cassette does not comprise an additional transit peptide in case in Table I, application no. 11, in column 6 in the same line as SEQ ID NO. 1061 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 19419, or a homolog or fragment thereof, or a homolog thereof being depicted in Table I, application no. 11, column 8, in the same line as SEQ ID NO. 19419, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 19419 is depicted, plastidic is mentioned, or a mitochondrial transit peptide in case in Table I, application no. 11, in column 6 in the same line as SEQ ID NO. 19419 is depicted, mitochondrial is mentioned; which is operable linked, too.

In another embodiment thereof the expression cassette does not comprise an additional transit peptide in case in Table I, application no. 11, in column 6 in the same line as SEQ ID NO. 19419 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 1623, or a homolog or fragment thereof, or a homolog thereof being depicted in Table I, application no. 11, column 8, in the same line as SEQ ID NO. 1623, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 1623 is depicted, plastidic is mentioned, or a mitochondrial transit peptide in case in Table I, application no. 11, in column 6 in the same line as SEQ ID NO. 1623 is depicted, mitochondrial is mentioned; which is operable linked, too.

In another embodiment thereof the expression cassette does not comprise an additional transit peptide in case in Table I, application no. 11, in column 6 in the same line as SEQ ID NO. 1623 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68657, or a homolog or fragment thereof, or a homolog thereof being depicted in Table I, application no. 11, column 8, in the same line as SEQ ID NO. 68657, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 68657 is depicted, plastidic is mentioned, or a mitochondrial transit peptide in case in Table I, application no. 11, in column 6 in the same line as SEQ ID NO. 68657 is depicted, mitochondrial is mentioned; which is operable linked, too.

In another embodiment thereof the expression cassette does not comprise an additional transit peptide in case in Table I, application no. 11, in column 6 in the same line as SEQ ID NO. 68657 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 22921, or a homolog or fragment thereof, or a homolog thereof being depicted in Table I, application no. 11, column 8, in the same line as SEQ ID NO. 22921, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 22921 is depicted, plastidic is mentioned, or a mitochondrial transit peptide in case in Table I, application no. 11, in column 6 in the same line as SEQ ID NO. 22921 is depicted, mitochondrial is mentioned; which is operable linked, too.

In another embodiment thereof the expression cassette does not comprise an additional transit peptide in case in Table I, application no. 11, in column 6 in the same line as SEQ ID NO. 22921 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 2935, or a homolog or fragment thereof, or a homolog thereof being depicted in Table I, application no. 11, column 8, in the same line as SEQ ID NO. 2935, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 2935 is depicted, plastidic is mentioned, or a mitochondrial transit peptide in case in Table I, application no. 11, in column 6 in the same line as SEQ ID NO. 2935 is depicted, mitochondrial is mentioned; which is operable linked, too.

In another embodiment thereof the expression cassette does not comprise an additional transit peptide in case in Table I, application no. 11, in column 6 in the same line as SEQ ID NO. 2935 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 3279, or a homolog or fragment thereof, or a homolog thereof being depicted in Table I, application no. 11, column 8, in the same line as SEQ ID NO. 3279, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 3279 is depicted, plastidic is mentioned, or a mitochondrial transit peptide in case in Table I, application no. 11, in column 6 in the same line as SEQ ID NO. 3279 is depicted, mitochondrial is mentioned; which is operable linked, too.

In another embodiment thereof the expression cassette does not comprise an additional transit peptide in case in Table I, application no. 11, in column 6 in the same line as SEQ ID NO. 3279 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 24232, or a homolog or fragment thereof, or a homolog thereof being depicted in Table I, application no. 11, column 8, in the same line as SEQ ID NO. 24232, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 24232 is depicted, plastidic is mentioned, or a mitochondrial transit peptide in case in Table I, application no. 11, in column 6 in the same line as SEQ ID NO. 24232 is depicted, mitochondrial is mentioned; which is operable linked, too.

In another embodiment thereof the expression cassette does not comprise an additional transit peptide in case in Table I, application no. 11, in column 6 in the same line as SEQ ID NO. 24232 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68849, or a homolog or fragment thereof, or a homolog thereof being depicted in Table I, application no. 11, column 8, in the same line as SEQ ID NO. 68849, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 68849 is depicted, plastidic is mentioned, or a mitochondrial transit peptide in case in Table I, application no. 11, in column 6 in the same line as SEQ ID NO. 68849 is depicted, mitochondrial is mentioned; which is operable linked, too.

In another embodiment thereof the expression cassette does not comprise an additional transit peptide in case in Table I, application no. 11, in column 6 in the same line as SEQ ID NO. 68849 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4102, or a homolog or fragment thereof, or a homolog thereof being depicted in Table I, application no. 11, column 8, in the same line as SEQ ID NO. 4102, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 4102 is depicted, plastidic is mentioned, or a mitochondrial transit peptide in case in Table I, application no. 11, in column 6 in the same line as SEQ ID NO. 4102 is depicted, mitochondrial is mentioned; which is operable linked, too.

In another embodiment thereof the expression cassette does not comprise an additional transit peptide in case in Table I, application no. 11, in column 6 in the same line as SEQ ID NO. 4102 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4348, or a homolog or fragment thereof, or a homolog thereof being depicted in Table I, application no. 11, column 8, in the same line as SEQ ID NO. 4348, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 4348 is depicted, plastidic is mentioned, or a mitochondrial transit peptide in case in Table I, application no. 11, in column 6 in the same line as SEQ ID NO. 4348 is depicted, mitochondrial is mentioned; which is operable linked, too.

In another embodiment thereof the expression cassette does not comprise an additional transit peptide in case in Table I, application no. 11, in column 6 in the same line as SEQ ID NO. 4348 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 5318, or a homolog or fragment thereof, or a homolog thereof being depicted in Table I, application no. 11, column 8, in the same line as SEQ ID NO. 5318, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 5318 is depicted, plastidic is mentioned, or a mitochondrial transit peptide in case in Table I, application no. 11, in column 6 in the same line as SEQ ID NO. 5318 is depicted, mitochondrial is mentioned; which is operable linked, too.

In another embodiment thereof the expression cassette does not comprise an additional transit peptide in case in Table I, application no. 11, in column 6 in the same line as SEQ ID NO. 5318 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 25498, or a homolog or fragment thereof, or a homolog thereof being depicted in Table I, application no. 11, column 8, in the same line as SEQ ID NO. 25498, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 25498 is depicted, plastidic is mentioned, or a mitochondrial transit peptide in case in Table I, application no. 11, in column 6 in the same line as SEQ ID NO. 25498 is depicted, mitochondrial is mentioned; which is operable linked, too.

In another embodiment thereof the expression cassette does not comprise an additional transit peptide in case in Table I, application no. 11, in column 6 in the same line as SEQ ID NO. 25498 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 114083, or a homolog or fragment thereof, or a homolog thereof being depicted in Table I, application no. 11, column 8, in the same line as SEQ ID NO. 114083, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 114083 is depicted, plastidic is mentioned, or a mitochondrial transit peptide in case in Table I, application no. 11, in column 6 in the same line as SEQ ID NO. 114083 is depicted, mitochondrial is mentioned; which is operable linked, too.

In another embodiment thereof the expression cassette does not comprise an additional transit peptide in case in Table I, application no. 11, in column 6 in the same line as SEQ ID NO. 114083 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 111155, or a homolog or fragment thereof, or a homolog thereof being depicted in Table I, application no. 11, column 8, in the same line as SEQ ID NO. 111155, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 111155 is depicted, plastidic is mentioned, or a mitochondrial transit peptide in case in Table I, application no. 11, in column 6 in the same line as SEQ ID NO. 111155 is depicted, mitochondrial is mentioned; which is operable linked, too.

In another embodiment thereof the expression cassette does not comprise an additional transit peptide in case in Table I, application no. 11, in column 6 in the same line as SEQ ID NO. 111155 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 114231, or a homolog or fragment thereof, or a homolog thereof being depicted in Table I, application no. 11, column 8, in the same line as SEQ ID NO. 114231, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 114231 is depicted, plastidic is mentioned, or a mitochondrial transit peptide in case in Table I, application no. 11, in column 6 in the same line as SEQ ID NO. 114231 is depicted, mitochondrial is mentioned; which is operable linked, too.

In another embodiment thereof the expression cassette does not comprise an additional transit peptide in case in Table I, application no. 11, in column 6 in the same line as SEQ ID NO. 114231 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 73719, or a homolog or fragment thereof, or a homolog thereof being depicted in Table I, application no. 11, column 8, in the same line as SEQ ID NO. 73719, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 73719 is depicted, plastidic is mentioned, or a mitochondrial transit peptide in case in Table I, application no. 11, in column 6 in the same line as SEQ ID NO. 73719 is depicted, mitochondrial is mentioned; which is operable linked, too.

In another embodiment thereof the expression cassette does not comprise an additional transit peptide in case in Table I, application no. 11, in column 6 in the same line as SEQ ID NO. 73719 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 32648, or a homolog or fragment thereof, or a homolog thereof being depicted in Table I, application no. 11, column 8, in the same line as SEQ ID NO. 32648, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 32648 is depicted, plastidic is mentioned, or a mitochondrial transit peptide in case in Table I, application no. 11, in column 6 in the same line as SEQ ID NO. 32648 is depicted, mitochondrial is mentioned; which is operable linked, too.

In another embodiment thereof the expression cassette does not comprise an additional transit peptide in case in Table I, application no. 11, in column 6 in the same line as SEQ ID NO. 32648 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6674, or a homolog or fragment thereof, or a homolog thereof being depicted in Table I, application no. 11, column 8, in the same line as SEQ ID NO. 6674, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 6674 is depicted, plastidic is mentioned, or a mitochondrial transit peptide in case in Table I, application no. 11, in column 6 in the same line as SEQ ID NO. 6674 is depicted, mitochondrial is mentioned; which is operable linked, too.

In another embodiment thereof the expression cassette does not comprise an additional transit peptide in case in Table I, application no. 11, in column 6 in the same line as SEQ ID NO. 6674 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 34044, or a homolog or fragment thereof, or a homolog thereof being depicted in Table I, application no. 11, column 8, in the same line as SEQ ID NO. 34044, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 34044 is depicted, plastidic is mentioned, or a mitochondrial transit peptide in case in Table I, application no. 11, in column 6 in the same line as SEQ ID NO. 34044 is depicted, mitochondrial is mentioned; which is operable linked, too.

In another embodiment thereof the expression cassette does not comprise an additional transit peptide in case in Table I, application no. 11, in column 6 in the same line as SEQ ID NO. 34044 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 103958, or a homolog or fragment thereof, or a homolog thereof being depicted in Table I, application no. 11, column 8, in the same line as SEQ ID NO. 103958, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 103958 is depicted, plastidic is mentioned, or a mitochondrial transit peptide in case in Table I, application no. 11, in column 6 in the same line as SEQ ID NO. 103958 is depicted, mitochondrial is mentioned; which is operable linked, too.

In another embodiment thereof the expression cassette does not comprise an additional transit peptide in case in Table I, application no. 11, in column 6 in the same line as SEQ ID NO. 103958 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 37400, or a homolog or fragment thereof, or a homolog thereof being depicted in Table I, application no. 11, column 8, in the same line as SEQ ID NO. 37400, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 37400 is depicted, plastidic is mentioned, or a mitochondrial transit peptide in case in Table I, application no. 11, in column 6 in the same line as SEQ ID NO. 37400 is depicted, mitochondrial is mentioned; which is operable linked, too.

In another embodiment thereof the expression cassette does not comprise an additional transit peptide in case in Table I, application no. 11, in column 6 in the same line as SEQ ID NO. 37400 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 112111, or a homolog or fragment thereof, or a homolog thereof being depicted in Table I, application no. 11, column 8, in the same line as SEQ ID NO. 112111, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 112111 is depicted, plastidic is mentioned, or a mitochondrial transit peptide in case in Table I, application no. 11, in column 6 in the same line as SEQ ID NO. 112111 is depicted, mitochondrial is mentioned; which is operable linked, too.

In another embodiment thereof the expression cassette does not comprise an additional transit peptide in case in Table I, application no. 11, in column 6 in the same line as SEQ ID NO. 112111 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 119135, or a homolog or fragment thereof, or a homolog thereof being depicted in Table I, application no. 11, column 8, in the same line as SEQ ID NO. 119135, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 119135 is depicted, plastidic is mentioned, or a mitochondrial transit peptide in case in Table I, application no. 11, in column 6 in the same line as SEQ ID NO. 119135 is depicted, mitochondrial is mentioned; which is operable linked, too.

In another embodiment thereof the expression cassette does not comprise an additional transit peptide in case in Table I, application no. 11, in column 6 in the same line as SEQ ID NO. 119135 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38226, or a homolog or fragment thereof, or a homolog thereof being depicted in Table I, application no. 11, column 8, in the same line as SEQ ID NO. 38226, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 38226 is depicted, plastidic is mentioned, or a mitochondrial transit peptide in case in Table I, application no. 11, in column 6 in the same line as SEQ ID NO. 38226 is depicted, mitochondrial is mentioned; which is operable linked, too.

In another embodiment thereof the expression cassette does not comprise an additional transit peptide in case in Table I, application no. 11, in column 6 in the same line as SEQ ID NO. 38226 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38266, or a homolog or fragment thereof, or a homolog thereof being depicted in Table I, application no. 11, column 8, in the same line as SEQ ID NO. 38266, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 38266 is depicted, plastidic is mentioned, or a mitochondrial transit peptide in case in Table I, application no. 11, in column 6 in the same line as SEQ ID NO. 38266 is depicted, mitochondrial is mentioned; which is operable linked, too.

In another embodiment thereof the expression cassette does not comprise an additional transit peptide in case in Table I, application no. 11, in column 6 in the same line as SEQ ID NO. 38266 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78154, or a homolog or fragment thereof, or a homolog thereof being depicted in Table I, application no. 11, column 8, in the same line as SEQ ID NO. 78154, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 78154 is depicted, plastidic is mentioned, or a mitochondrial transit peptide in case in Table I, application no. 11, in column 6 in the same line as SEQ ID NO. 78154 is depicted, mitochondrial is mentioned; which is operable linked, too.

In another embodiment thereof the expression cassette does not comprise an additional transit peptide in case in Table I, application no. 11, in column 6 in the same line as SEQ ID NO. 78154 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38300, or a homolog or fragment thereof, or a homolog thereof being depicted in Table I, application no. 11, column 8, in the same line as SEQ ID NO. 38300, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 38300 is depicted, plastidic is mentioned, or a mitochondrial transit peptide in case in Table I, application no. 11, in column 6 in the same line as SEQ ID NO. 38300 is depicted, mitochondrial is mentioned; which is operable linked, too.

In another embodiment thereof the expression cassette does not comprise an additional transit peptide in case in Table I, application no. 11, in column 6 in the same line as SEQ ID NO. 38300 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78753, or a homolog or fragment thereof, or a homolog thereof being depicted in Table I, application no. 11, column 8, in the same line as SEQ ID NO. 78753, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 78753 is depicted, plastidic is mentioned, or a mitochondrial transit peptide in case in Table I, application no. 11, in column 6 in the same line as SEQ ID NO. 78753 is depicted, mitochondrial is mentioned; which is operable linked, too.

In another embodiment thereof the expression cassette does not comprise an additional transit peptide in case in Table I, application no. 11, in column 6 in the same line as SEQ ID NO. 78753 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78953, or a homolog or fragment thereof, or a homolog thereof being depicted in Table I, application no. 11, column 8, in the same line as SEQ ID NO. 78953, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 78953 is depicted, plastidic is mentioned, or a mitochondrial transit peptide in case in Table I, application no. 11, in column 6 in the same line as SEQ ID NO. 78953 is depicted, mitochondrial is mentioned; which is operable linked, too.

In another embodiment thereof the expression cassette does not comprise an additional transit peptide in case in Table I, application no. 11, in column 6 in the same line as SEQ ID NO. 78953 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 8920, or a homolog or fragment thereof, or a homolog thereof being depicted in Table I, application no. 11, column 8, in the same line as SEQ ID NO. 8920, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 8920 is depicted, plastidic is mentioned, or a mitochondrial transit peptide in case in Table I, application no. 11, in column 6 in the same line as SEQ ID NO. 8920 is depicted, mitochondrial is mentioned; which is operable linked, too.

In another embodiment thereof the expression cassette does not comprise an additional transit peptide in case in Table I, application no. 11, in column 6 in the same line as SEQ ID NO. 8920 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 112193, or a homolog or fragment thereof, or a homolog thereof being depicted in Table I, application no. 11, column 8, in the same line as SEQ ID NO. 112193, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 112193 is depicted, plastidic is mentioned, or a mitochondrial transit peptide in case in Table I, application no. 11, in column 6 in the same line as SEQ ID NO. 112193 is depicted, mitochondrial is mentioned; which is operable linked, too.

In another embodiment thereof the expression cassette does not comprise an additional transit peptide in case in Table I, application no. 11, in column 6 in the same line as SEQ ID NO. 112193 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115595, or a homolog or fragment thereof, or a homolog thereof being depicted in Table I, application no. 11, column 8, in the same line as SEQ ID NO. 115595, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 115595 is depicted, plastidic is mentioned, or a mitochondrial transit peptide in case in Table I, application no. 11, in column 6 in the same line as SEQ ID NO. 115595 is depicted, mitochondrial is mentioned; which is operable linked, too.

In another embodiment thereof the expression cassette does not comprise an additional transit peptide in case in Table I, application no. 11, in column 6 in the same line as SEQ ID NO. 115595 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39002, or a homolog or fragment thereof, or a homolog thereof being depicted in Table I, application no. 11, column 8, in the same line as SEQ ID NO. 39002, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 39002 is depicted, plastidic is mentioned, or a mitochondrial transit peptide in case in Table I, application no. 11, in column 6 in the same line as SEQ ID NO. 39002 is depicted, mitochondrial is mentioned; which is operable linked, too.

In another embodiment thereof the expression cassette does not comprise an additional transit peptide in case in Table I, application no. 11, in column 6 in the same line as SEQ ID NO. 39002 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39237, or a homolog or fragment thereof, or a homolog thereof being depicted in Table I, application no. 11, column 8, in the same line as SEQ ID NO. 39237, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 39237 is depicted, plastidic is mentioned, or a mitochondrial transit peptide in case in Table I, application no. 11, in column 6 in the same line as SEQ ID NO. 39237 is depicted, mitochondrial is mentioned; which is operable linked, too.

In another embodiment thereof the expression cassette does not comprise an additional transit peptide in case in Table I, application no. 11, in column 6 in the same line as SEQ ID NO. 39237 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115628, or a homolog or fragment thereof, or a homolog thereof being depicted in Table I, application no. 11, column 8, in the same line as SEQ ID NO. 115628, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 115628 is depicted, plastidic is mentioned, or a mitochondrial transit peptide in case in Table I, application no. 11, in column 6 in the same line as SEQ ID NO. 115628 is depicted, mitochondrial is mentioned; which is operable linked, too.

In another embodiment thereof the expression cassette does not comprise an additional transit peptide in case in Table I, application no. 11, in column 6 in the same line as SEQ ID NO. 115628 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115655, or a homolog or fragment thereof, or a homolog thereof being depicted in Table I, application no. 11, column 8, in the same line as SEQ ID NO. 115655, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 115655 is depicted, plastidic is mentioned, or a mitochondrial transit peptide in case in Table I, application no. 11, in column 6 in the same line as SEQ ID NO. 115655 is depicted, mitochondrial is mentioned; which is operable linked, too.

In another embodiment thereof the expression cassette does not comprise an additional transit peptide in case in Table I, application no. 11, in column 6 in the same line as SEQ ID NO. 115655 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 81075, or a homolog or fragment thereof, or a homolog thereof being depicted in Table I, application no. 11, column 8, in the same line as SEQ ID NO. 81075, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 81075 is depicted, plastidic is mentioned, or a mitochondrial transit peptide in case in Table I, application no. 11, in column 6 in the same line as SEQ ID NO. 81075 is depicted, mitochondrial is mentioned; which is operable linked, too.

In another embodiment thereof the expression cassette does not comprise an additional transit peptide in case in Table I, application no. 11, in column 6 in the same line as SEQ ID NO. 81075 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115719, or a homolog or fragment thereof, or a homolog thereof being depicted in Table I, application no. 11, column 8, in the same line as SEQ ID NO. 115719, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 115719 is depicted, plastidic is mentioned, or a mitochondrial transit peptide in case in Table I, application no. 11, in column 6 in the same line as SEQ ID NO. 115719 is depicted, mitochondrial is mentioned; which is operable linked, too.

In another embodiment thereof the expression cassette does not comprise an additional transit peptide in case in Table I, application no. 11, in column 6 in the same line as SEQ ID NO. 115719 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115791, or a homolog or fragment thereof, or a homolog thereof being depicted in Table I, application no. 11, column 8, in the same line as SEQ ID NO. 115791, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 115791 is depicted, plastidic is mentioned, or a mitochondrial transit peptide in case in Table I, application no. 11, in column 6 in the same line as SEQ ID NO. 115791 is depicted, mitochondrial is mentioned; which is operable linked, too.

In another embodiment thereof the expression cassette does not comprise an additional transit peptide in case in Table I, application no. 11, in column 6 in the same line as SEQ ID NO. 115791 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 81980, or a homolog or fragment thereof, or a homolog thereof being depicted in Table I, application no. 11, column 8, in the same line as SEQ ID NO. 81980, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 81980 is depicted, plastidic is mentioned, or a mitochondrial transit peptide in case in Table I, application no. 11, in column 6 in the same line as SEQ ID NO. 81980 is depicted, mitochondrial is mentioned; which is operable linked, too.

In another embodiment thereof the expression cassette does not comprise an additional transit peptide in case in Table I, application no. 11, in column 6 in the same line as SEQ ID NO. 81980 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 44196, or a homolog or fragment thereof, or a homolog thereof being depicted in Table I, application no. 11, column 8, in the same line as SEQ ID NO. 44196, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 44196 is depicted, plastidic is mentioned, or a mitochondrial transit peptide in case in Table I, application no. 11, in column 6 in the same line as SEQ ID NO. 44196 is depicted, mitochondrial is mentioned; which is operable linked, too.

In another embodiment thereof the expression cassette does not comprise an additional transit peptide in case in Table I, application no. 11, in column 6 in the same line as SEQ ID NO. 44196 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 10726, or a homolog or fragment thereof, or a homolog thereof being depicted in Table I, application no. 11, column 8, in the same line as SEQ ID NO. 10726, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 10726 is depicted, plastidic is mentioned, or a mitochondrial transit peptide in case in Table I, application no. 11, in column 6 in the same line as SEQ ID NO. 10726 is depicted, mitochondrial is mentioned; which is operable linked, too.

In another embodiment thereof the expression cassette does not comprise an additional transit peptide in case in Table I, application no. 11, in column 6 in the same line as SEQ ID NO. 10726 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 100675, or a homolog or fragment thereof, or a homolog thereof being depicted in Table I, application no. 11, column 8, in the same line as SEQ ID NO. 100675, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 100675 is depicted, plastidic is mentioned, or a mitochondrial transit peptide in case in Table I, application no. 11, in column 6 in the same line as SEQ ID NO. 100675 is depicted, mitochondrial is mentioned; which is operable linked, too.

In another embodiment thereof the expression cassette does not comprise an additional transit peptide in case in Table I, application no. 11, in column 6 in the same line as SEQ ID NO. 100675 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 10811, or a homolog or fragment thereof, or a homolog thereof being depicted in Table I, application no. 11, column 8, in the same line as SEQ ID NO. 10811, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 10811 is depicted, plastidic is mentioned, or a mitochondrial transit peptide in case in Table I, application no. 11, in column 6 in the same line as SEQ ID NO. 10811 is depicted, mitochondrial is mentioned; which is operable linked, too.

In another embodiment thereof the expression cassette does not comprise an additional transit peptide in case in Table I, application no. 11, in column 6 in the same line as SEQ ID NO. 10811 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 46751, or a homolog or fragment thereof, or a homolog thereof being depicted in Table I, application no. 11, column 8, in the same line as SEQ ID NO. 46751, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 46751 is depicted, plastidic is mentioned, or a mitochondrial transit peptide in case in Table I, application no. 11, in column 6 in the same line as SEQ ID NO. 46751 is depicted, mitochondrial is mentioned; which is operable linked, too.

In another embodiment thereof the expression cassette does not comprise an additional transit peptide in case in Table I, application no. 11, in column 6 in the same line as SEQ ID NO. 46751 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 106920, or a homolog or fragment thereof, or a homolog thereof being depicted in Table I, application no. 11, column 8, in the same line as SEQ ID NO. 106920, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 106920 is depicted, plastidic is mentioned, or a mitochondrial transit peptide in case in Table I, application no. 11, in column 6 in the same line as SEQ ID NO. 106920 is depicted, mitochondrial is mentioned; which is operable linked, too.

In another embodiment thereof the expression cassette does not comprise an additional transit peptide in case in Table I, application no. 11, in column 6 in the same line as SEQ ID NO. 106920 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 94741, or a homolog or fragment thereof, or a homolog thereof being depicted in Table I, application no. 11, column 8, in the same line as SEQ ID NO. 94741, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 94741 is depicted, plastidic is mentioned, or a mitochondrial transit peptide in case in Table I, application no. 11, in column 6 in the same line as SEQ ID NO. 94741 is depicted, mitochondrial is mentioned; which is operable linked, too.

In another embodiment thereof the expression cassette does not comprise an additional transit peptide in case in Table I, application no. 11, in column 6 in the same line as SEQ ID NO. 94741 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 52634, or a homolog or fragment thereof, or a homolog thereof being depicted in Table I, application no. 11, column 8, in the same line as SEQ ID NO. 52634, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 52634 is depicted, plastidic is mentioned, or a mitochondrial transit peptide in case in Table I, application no. 11, in column 6 in the same line as SEQ ID NO. 52634 is depicted, mitochondrial is mentioned; which is operable linked, too.

In another embodiment thereof the expression cassette does not comprise an additional transit peptide in case in Table I, application no. 11, in column 6 in the same line as SEQ ID NO. 52634 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 85723, or a homolog or fragment thereof, or a homolog thereof being depicted in Table I, application no. 11, column 8, in the same line as SEQ ID NO. 85723, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 85723 is depicted, plastidic is mentioned, or a mitochondrial transit peptide in case in Table I, application no. 11, in column 6 in the same line as SEQ ID NO. 85723 is depicted, mitochondrial is mentioned; which is operable linked, too.

In another embodiment thereof the expression cassette does not comprise an additional transit peptide in case in Table I, application no. 11, in column 6 in the same line as SEQ ID NO. 85723 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 52660, or a homolog or fragment thereof, or a homolog thereof being depicted in Table I, application no. 11, column 8, in the same line as SEQ ID NO. 52660, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 52660 is depicted, plastidic is mentioned, or a mitochondrial transit peptide in case in Table I, application no. 11, in column 6 in the same line as SEQ ID NO. 52660 is depicted, mitochondrial is mentioned; which is operable linked, too.

In another embodiment thereof the expression cassette does not comprise an additional transit peptide in case in Table I, application no. 11, in column 6 in the same line as SEQ ID NO. 52660 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 54337, or a homolog or fragment thereof, or a homolog thereof being depicted in Table I, application no. 11, column 8, in the same line as SEQ ID NO. 54337, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 54337 is depicted, plastidic is mentioned, or a mitochondrial transit peptide in case in Table I, application no. 11, in column 6 in the same line as SEQ ID NO. 54337 is depicted, mitochondrial is mentioned; which is operable linked, too.

In another embodiment thereof the expression cassette does not comprise an additional transit peptide in case in Table I, application no. 11, in column 6 in the same line as SEQ ID NO. 54337 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 95047, or a homolog or fragment thereof, or a homolog thereof being depicted in Table I, application no. 11, column 8, in the same line as SEQ ID NO. 95047, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 95047 is depicted, plastidic is mentioned, or a mitochondrial transit peptide in case in Table I, application no. 11, in column 6 in the same line as SEQ ID NO. 95047 is depicted, mitochondrial is mentioned; which is operable linked, too.

In another embodiment thereof the expression cassette does not comprise an additional transit peptide in case in Table I, application no. 11, in column 6 in the same line as SEQ ID NO. 95047 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 11423, or a homolog or fragment thereof, or a homolog thereof being depicted in Table I, application no. 11, column 8, in the same line as SEQ ID NO. 11423, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 11423 is depicted, plastidic is mentioned, or a mitochondrial transit peptide in case in Table I, application no. 11, in column 6 in the same line as SEQ ID NO. 11423 is depicted, mitochondrial is mentioned; which is operable linked, too.

In another embodiment thereof the expression cassette does not comprise an additional transit peptide in case in Table I, application no. 11, in column 6 in the same line as SEQ ID NO. 11423 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 85743, or a homolog or fragment thereof, or a homolog thereof being depicted in Table I, application no. 11, column 8, in the same line as SEQ ID NO. 85743, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 85743 is depicted, plastidic is mentioned, or a mitochondrial transit peptide in case in Table I, application no. 11, in column 6 in the same line as SEQ ID NO. 85743 is depicted, mitochondrial is mentioned; which is operable linked, too.

In another embodiment thereof the expression cassette does not comprise an additional transit peptide in case in Table I, application no. 11, in column 6 in the same line as SEQ ID NO. 85743 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 86447, or a homolog or fragment thereof, or a homolog thereof being depicted in Table I, application no. 11, column 8, in the same line as SEQ ID NO. 86447, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 86447 is depicted, plastidic is mentioned, or a mitochondrial transit peptide in case in Table I, application no. 11, in column 6 in the same line as SEQ ID NO. 86447 is depicted, mitochondrial is mentioned; which is operable linked, too.

In another embodiment thereof the expression cassette does not comprise an additional transit peptide in case in Table I, application no. 11, in column 6 in the same line as SEQ ID NO. 86447 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 11471, or a homolog or fragment thereof, or a homolog thereof being depicted in Table I, application no. 11, column 8, in the same line as SEQ ID NO. 11471, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 11471 is depicted, plastidic is mentioned, or a mitochondrial transit peptide in case in Table I, application no. 11, in column 6 in the same line as SEQ ID NO. 11471 is depicted, mitochondrial is mentioned; which is operable linked, too.

In another embodiment thereof the expression cassette does not comprise an additional transit peptide in case in Table I, application no. 11, in column 6 in the same line as SEQ ID NO. 11471 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 55385, or a homolog or fragment thereof, or a homolog thereof being depicted in Table I, application no. 11, column 8, in the same line as SEQ ID NO. 55385, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 55385 is depicted, plastidic is mentioned, or a mitochondrial transit peptide in case in Table I, application no. 11, in column 6 in the same line as SEQ ID NO. 55385 is depicted, mitochondrial is mentioned; which is operable linked, too.

In another embodiment thereof the expression cassette does not comprise an additional transit peptide in case in Table I, application no. 11, in column 6 in the same line as SEQ ID NO. 55385 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 119222, or a homolog or fragment thereof, or a homolog thereof being depicted in Table I, application no. 11, column 8, in the same line as SEQ ID NO. 119222, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 119222 is depicted, plastidic is mentioned, or a mitochondrial transit peptide in case in Table I, application no. 11, in column 6 in the same line as SEQ ID NO. 119222 is depicted, mitochondrial is mentioned; which is operable linked, too.

In another embodiment thereof the expression cassette does not comprise an additional transit peptide in case in Table I, application no. 11, in column 6 in the same line as SEQ ID NO. 119222 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 116341, or a homolog or fragment thereof, or a homolog thereof being depicted in Table I, application no. 11, column 8, in the same line as SEQ ID NO. 116341, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 116341 is depicted, plastidic is mentioned, or a mitochondrial transit peptide in case in Table I, application no. 11, in column 6 in the same line as SEQ ID NO. 116341 is depicted, mitochondrial is mentioned; which is operable linked, too.

In another embodiment thereof the expression cassette does not comprise an additional transit peptide in case in Table I, application no. 11, in column 6 in the same line as SEQ ID NO. 116341 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 116460, or a homolog or fragment thereof, or a homolog thereof being depicted in Table I, application no. 11, column 8, in the same line as SEQ ID NO. 116460, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 116460 is depicted, plastidic is mentioned, or a mitochondrial transit peptide in case in Table I, application no. 11, in column 6 in the same line as SEQ ID NO. 116460 is depicted, mitochondrial is mentioned; which is operable linked, too.

In another embodiment thereof the expression cassette does not comprise an additional transit peptide in case in Table I, application no. 11, in column 6 in the same line as SEQ ID NO. 116460 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 58324, or a homolog or fragment thereof, or a homolog thereof being depicted in Table I, application no. 11, column 8, in the same line as SEQ ID NO. 58324, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 58324 is depicted, plastidic is mentioned, or a mitochondrial transit peptide in case in Table I, application no. 11, in column 6 in the same line as SEQ ID NO. 58324 is depicted, mitochondrial is mentioned; which is operable linked, too.

In another embodiment thereof the expression cassette does not comprise an additional transit peptide in case in Table I, application no. 11, in column 6 in the same line as SEQ ID NO. 58324 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12140, or a homolog or fragment thereof, or a homolog thereof being depicted in Table I, application no. 11, column 8, in the same line as SEQ ID NO. 12140, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 12140 is depicted, plastidic is mentioned, or a mitochondrial transit peptide in case in Table I, application no. 11, in column 6 in the same line as SEQ ID NO. 12140 is depicted, mitochondrial is mentioned; which is operable linked, too.

In another embodiment thereof the expression cassette does not comprise an additional transit peptide in case in Table I, application no. 11, in column 6 in the same line as SEQ ID NO. 12140 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12341, or a homolog or fragment thereof, or a homolog thereof being depicted in Table I, application no. 11, column 8, in the same line as SEQ ID NO. 12341, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 12341 is depicted, plastidic is mentioned, or a mitochondrial transit peptide in case in Table I, application no. 11, in column 6 in the same line as SEQ ID NO. 12341 is depicted, mitochondrial is mentioned; which is operable linked, too.

In another embodiment thereof the expression cassette does not comprise an additional transit peptide in case in Table I, application no. 11, in column 6 in the same line as SEQ ID NO. 12341 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 61553, or a homolog or fragment thereof, or a homolog thereof being depicted in Table I, application no. 11, column 8, in the same line as SEQ ID NO. 61553, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 61553 is depicted, plastidic is mentioned, or a mitochondrial transit peptide in case in Table I, application no. 11, in column 6 in the same line as SEQ ID NO. 61553 is depicted, mitochondrial is mentioned; which is operable linked, too.

In another embodiment thereof the expression cassette does not comprise an additional transit peptide in case in Table I, application no. 11, in column 6 in the same line as SEQ ID NO. 61553 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12974, or a homolog or fragment thereof, or a homolog thereof being depicted in Table I, application no. 11, column 8, in the same line as SEQ ID NO. 12974, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 12974 is depicted, plastidic is mentioned, or a mitochondrial transit peptide in case in Table I, application no. 11, in column 6 in the same line as SEQ ID NO. 12974 is depicted, mitochondrial is mentioned; which is operable linked, too.

In another embodiment thereof the expression cassette does not comprise an additional transit peptide in case in Table I, application no. 11, in column 6 in the same line as SEQ ID NO. 12974 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 119347, or a homolog or fragment thereof, or a homolog thereof being depicted in Table I, application no. 11, column 8, in the same line as SEQ ID NO. 119347, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 119347 is depicted, plastidic is mentioned, or a mitochondrial transit peptide in case in Table I, application no. 11, in column 6 in the same line as SEQ ID NO. 119347 is depicted, mitochondrial is mentioned; which is operable linked, too.

In another embodiment thereof the expression cassette does not comprise an additional transit peptide in case in Table I, application no. 11, in column 6 in the same line as SEQ ID NO. 119347 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 119408, or a homolog or fragment thereof, or a homolog thereof being depicted in Table I, application no. 11, column 8, in the same line as SEQ ID NO. 119408, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 119408 is depicted, plastidic is mentioned, or a mitochondrial transit peptide in case in Table I, application no. 11, in column 6 in the same line as SEQ ID NO. 119408 is depicted, mitochondrial is mentioned; which is operable linked, too.

In another embodiment thereof the expression cassette does not comprise an additional transit peptide in case in Table I, application no. 11, in column 6 in the same line as SEQ ID NO. 119408 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 122518, or a homolog or fragment thereof, or a homolog thereof being depicted in Table I, application no. 11, column 8, in the same line as SEQ ID NO. 122518, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 122518 is depicted, plastidic is mentioned, or a mitochondrial transit peptide in case in Table I, application no. 11, in column 6 in the same line as SEQ ID NO. 122518 is depicted, mitochondrial is mentioned; which is operable linked, too.

In another embodiment thereof the expression cassette does not comprise an additional transit peptide in case in Table I, application no. 11, in column 6 in the same line as SEQ ID NO. 122518 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 14706, or a homolog or fragment thereof, or a homolog thereof being depicted in Table I, application no. 11, column 8, in the same line as SEQ ID NO. 14706, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 14706 is depicted, plastidic is mentioned, or a mitochondrial transit peptide in case in Table I, application no. 11, in column 6 in the same line as SEQ ID NO. 14706 is depicted, mitochondrial is mentioned; which is operable linked, too.

In another embodiment thereof the expression cassette does not comprise an additional transit peptide in case in Table I, application no. 11, in column 6 in the same line as SEQ ID NO. 14706 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 64964, or a homolog or fragment thereof, or a homolog thereof being depicted in Table I, application no. 11, column 8, in the same line as SEQ ID NO. 64964, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 64964 is depicted, plastidic is mentioned, or a mitochondrial transit peptide in case in Table I, application no. 11, in column 6 in the same line as SEQ ID NO. 64964 is depicted, mitochondrial is mentioned; which is operable linked, too.

In another embodiment thereof the expression cassette does not comprise an additional transit peptide in case in Table I, application no. 11, in column 6 in the same line as SEQ ID NO. 64964 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 66772, or a homolog or fragment thereof, or a homolog thereof being depicted in Table I, application no. 11, column 8, in the same line as SEQ ID NO. 66772, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 66772 is depicted, plastidic is mentioned, or a mitochondrial transit peptide in case in Table I, application no. 11, in column 6 in the same line as SEQ ID NO. 66772 is depicted, mitochondrial is mentioned; which is operable linked, too.

In another embodiment thereof the expression cassette does not comprise an additional transit peptide in case in Table I, application no. 11, in column 6 in the same line as SEQ ID NO. 66772 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 91958, or a homolog or fragment thereof, or a homolog thereof being depicted in Table I, application no. 11, column 8, in the same line as SEQ ID NO. 91958, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 91958 is depicted, plastidic is mentioned, or a mitochondrial transit peptide in case in Table I, application no. 11, in column 6 in the same line as SEQ ID NO. 91958 is depicted, mitochondrial is mentioned; which is operable linked, too.

In another embodiment thereof the expression cassette does not comprise an additional transit peptide in case in Table I, application no. 11, in column 6 in the same line as SEQ ID NO. 91958 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 118212, or a homolog or fragment thereof, or a homolog thereof being depicted in Table I, application no. 11, column 8, in the same line as SEQ ID NO. 118212, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 118212 is depicted, plastidic is mentioned, or a mitochondrial transit peptide in case in Table I, application no. 11, in column 6 in the same line as SEQ ID NO. 118212 is depicted, mitochondrial is mentioned; which is operable linked, too.

In another embodiment thereof the expression cassette does not comprise an additional transit peptide in case in Table I, application no. 11, in column 6 in the same line as SEQ ID NO. 118212 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 69, or a homolog or fragment thereof, or a homolog thereof being depicted in Table I, application no. 11, column 8, in the same line as SEQ ID NO. 69, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 69 is depicted, plastidic is mentioned, or a mitochondrial transit peptide in case in Table I, application no. 11, in column 6 in the same line as SEQ ID NO. 69 is depicted, mitochondrial is mentioned; which is operable linked, too.

In another embodiment thereof the expression cassette does not comprise an additional transit peptide in case in Table I, application no. 11, in column 6 in the same line as SEQ ID NO. 69 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 16883, or a homolog or fragment thereof, or a homolog thereof being depicted in Table I, application no. 11, column 8, in the same line as SEQ ID NO. 16883, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 11, in column 6 in the same line as SEQ ID NO. 16883 is depicted, plastidic is mentioned, or a mitochondrial transit peptide in case in Table I, application no. 11, in column 6 in the same line as SEQ ID NO. 16883 is depicted, mitochondrial is mentioned; which is operable linked, too.

In another embodiment thereof the expression cassette does not comprise an additional transit peptide in case in Table I, application no. 11, in column 6 in the same line as SEQ ID NO. 16883 is depicted, non-targeted is mentioned.

[0165.1.8.11] to [0170.1.8.11] for the disclosure of these paragraphs see [0165.1.8.8] to [0170.1.8.8] above.

The invention further provides a vector, comprising a nucleic acid molecule comprising 1) a nucleic acid molecule selected from the group consisting of:
(a) a nucleic acid molecule encoding a polypeptide shown in Table II, column 5 or 8, application no. 11, preferably shown in Table II A, application no. 11, in column 5, or in Table II A, application no. 11, column 8, or in Table II B, application no. 11, column 8, or a homolog or a fragment thereof;
(b) a nucleic acid molecule shown in Table I, application no. 11, column 5 or 8, preferably shown in Table I A, application no. 11, in column 5, or in Table I A, application no. 11, column 8, or in Table I B, application no. 11, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 11, preferably shown in Table II A, application no. 11, in column 5, or in Table II A, application no. 11, column 8, or in Table II B, application no. 11, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 11, preferably shown in Table I A, application no. 11, in column 5, or in Table I A, application no. 11, column 8, or in Table I B, application no. 11, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 11;
(i) a nucleic acid molecule encoding a polypeptide having the activity represented by a protein as depicted in column 5 of Table II, application no. 11;
(j) a nucleic acid molecule comprising a polynucleotide which is obtained by amplifying a cDNA library or a genomic library using the primers in Table III, application no. 11, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 11.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 11, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 11.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 11, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 11, in the same line as of the respective nucleic acid molecule according to 1) “plastidic” is depicted, or encoding a mitochondrial transit peptide, especially in case in column 6 of Table I, application no. 11, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 11, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 11, in the same line as of the respective nucleic acid molecule according to 1) “plastidic” is depicted, or encoding a mitochondrial transit peptide, especially in case in column 6 of Table I, application no. 11, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operable linked; a nucleic acid molecule encoding a transit peptide, especially in case in column 6 of Table I, application no. 11, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 11, in the same line as of the respective nucleic acid molecule according to 1) “plastidic” is depicted, or encoding a mitochondrial transit peptide, especially in case in column 6 of Table I, application no. 11, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 11 (in the same line).

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 11, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 11.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 11, in the same line as of the respective nucleic acid molecule according to 1) “plastidic” is depicted, or encoding a mitochondrial transit peptide, especially in case in column 6 of Table I, application no. 11, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 11, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 11, in the same line as of the respective nucleic acid molecule according to 1) “plastidic” is depicted, or encoding a mitochondrial transit peptide, especially in case in column 6 of Table I, application no. 11, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 11 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 11, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 11 (in the same line) and confers a generation or an increase of the respective fine chemical in a non-human organism or a part thereof as compared to a corresponding wild-type non-human organism or part thereof.

In a preferred embodiment of said vector said nucleic acid molecule according to 1 (a), (b), (c), (d), (e), (f), (g), (h), (i), (j) or (k), said promoter and optionally said nucleic acid molecule encoding a transit peptide are encompassed in an expression cassette. Accordingly in an embodiment the invention provides a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

The invention further provides a vector comprising a nucleic acid molecule comprising 1) a nucleic acid molecule selected from the group consisting of:
(a) a nucleic acid molecule encoding a polypeptide shown in Table II, column 5 or 8, application no. 11, preferably shown in Table II A, application no. 11, in column 5, or in Table II A, application no. 11, column 8, or in Table II B, application no. 11, column 8, or a homolog or a fragment thereof;
(b) a nucleic acid molecule shown in Table I, application no. 11, column 5 or 8, preferably shown in Table I A, application no. 11, in column 5, or in Table I A, application no. 11, column 8, or in Table I B, application no. 11, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 11, preferably shown in Table II A, application no. 11, in column 5, or in Table II A, application no. 11, column 8, or in Table II B, application no. 11, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 11, preferably shown in Table I A, application no. 11, in column 5, or in Table I A, application no. 11, column 8, or in Table I B, application no. 11, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 11;
(i) a nucleic acid molecule encoding a polypeptide having the activity represented by a protein as depicted in column 5 of Table II, application no. 11;
(j) a nucleic acid molecule comprising a polynucleotide which is obtained by amplifying a cDNA library or a genomic library using the primers in Table III, application no. 11, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto;
   whereby, preferably, the nucleic acid molecule according to 1 (a), (b), (c), (d), (e), (f), (g), (h), (i), (j) or (k) distinguishes over the sequence depicted in Table I A and/or I B, application no. 11, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 11, column 5 or 8, or the coding region thereof, respectively. In another embodiment, the nucleic acid molecule of the present invention, preferably the coding region thereof, is 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 11, column 5 or 8, or the coding region thereof, respectively. In a further embodiment the nucleic acid molecule does not encode the polypeptide sequence shown in Table II A and/or II B, application no. 11, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 11, column 5 or 8 and does not encode a protein of the sequence shown in Table II A and/or II B, application no. 11, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 11, column 5 and 8, or the coding region thereof. In a further embodiment, the protein of the present invention is at least 30%,40%, 50%, 60%, 70%, 80%, 90%, 95% identical to protein sequence depicted in Table II A and/or II B, application no. 11, column 5 or 8 but less than 100%, preferably less than 99.999%, 99.99% or 99.9%, more preferably less than 99%, 985, 97%, 96% or 95% identical to the sequence shown in Table II A and/or II B, application no. 11, column 5 or 8.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 11.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 11, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 11.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 11, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 11, in the same line as of the respective nucleic acid molecule according to 1) “plastidic” is depicted, or encoding a mitochondrial transit peptide, especially in case in column 6 of Table I, application no. 11, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 11, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 11, in the same line as of the respective nucleic acid molecule according to 1) “plastidic” is depicted, or encoding a mitochondrial transit peptide, especially in case in column 6 of Table I, application no. 11, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 11, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 11, in the same line as of the respective nucleic acid molecule according to 1) “plastidic” is depicted, or encoding a mitochondrial transit peptide, especially in case in column 6 of Table I, application no. 11, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 11.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 11, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 11.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 11, in the same line as of the respective nucleic acid molecule according to 1) “plastidic” is depicted, or encoding a mitochondrial transit peptide, especially in case in column 6 of Table I, application no. 11, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 11, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 11, in the same line as of the respective nucleic acid molecule according to 1) “plastidic” is depicted, or encoding a mitochondrial transit peptide, especially in case in column 6 of Table I, application no. 11, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 11 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 11, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 11 and confers a generation or an increase of the respective fine chemical in a non-human organism or a part thereof as compared to a corresponding wild-type non-human organism or part thereof.

[0181.1.8.11] to [0209.1.8.11] for the disclosure of these paragraphs see [0181.1.8.8] to [0209.1.8.8] above.

The genes of the invention, coding for an activity selected from the group consisting of 2-C-methyl-D-erythritol 4-phosphate cytidylyltransferase, 3-ketoacyl-CoA thiolase, 49747384_SOYBEAN-protein, AAS bifunctional protein, ABC metal ion transporter substrate-binding protein, acid phosphatase precursor, acid shock protein, aconitate hydratase, acyl transferase, arginine decarboxylase, aromatic acid decarboxylase, At1g19800-protein, At1g23110-protein, At2g45420-protein, ATPase epsilon subunit, b0050-protein, b1234-protein, b1248-protein, b1330-protein, b1670-protein, b1837-protein, b2032-protein, b2099-protein, b2107-protein, b2399-protein, b2682-protein, b2849-protein, b3814-protein, bifunctional protein (phosphoribosyltransferase and regulatory protein), branched chain amino acid ABC transporter ATP-binding protein, calcium-dependent protein kinase, carbamoyl-phosphate synthase subunit, carbon dioxide concentrating mechanism protein, cation-transporting ATPase, CCAAT-binding transcription factor, chlorophyllase, colanic acid biosynthesis protein, coproporphyrinogen III oxidase, DnaJ-like chaperone, ethanolamine utilization protein, F-box protein, Fe—S subunit of oxidoreductase, flavodoxin, glucose-6-phosphate 1-dehydrogenase, glutaredoxin, glutathione S-transferase, glycerol-3-phosphate dehydrogenase, heat shock transcription factor, HesB/YadR/YfhF family protein, homocitrate synthase, lipoprotein precursor, metal-dependent hydrolase, monothiol glutaredoxin, NADH-quinone oxidoreductase subunit, nitrate/nitrite transport protein, oxidoreductase, peptidyl-prolyl cis-trans isomerase, permease protein of ABC transporter, phosphate import ATP-binding protein, phosphoadenosine phosphosulfate reductase , phosphoribosylformyl glycinamidine synthase subunit, protein kinase, protein phosphatase, purine nucleoside phosphorylase, recombinase A, sll1032-protein, sodium/proton antiporter, ThiF family protein, threonine dehydratase, threonine efflux protein, transcription factor, and ymr013c-protein are also called “FCRP genes”.

[0211.1.8.11] to [0225.1.8.11] for the disclosure of these paragraphs see [0211.1.8.8] to [0225.1.8.8] above.

In addition to the sequence mentioned in Table I, application no. 11, column 5 or 8, preferably the coding region thereof, or its homologs or fragments, it may be advantageous additionally to express and/or mutate further genes in the non-human organisms. Especially advantageously, additionally at least one further gene of the biosynthetic pathway of the fine chemical is expressed in the non-human organisms such as plants or microorganisms. It is also possible that the regulation of the natural genes has been modified advantageously so that the gene and/or its gene product is no longer subject to the regulatory mechanisms which exist in the non-human organisms. This leads to an increased synthesis of the respective fatty acid since, for example, feedback regulations no longer exist to the same extent or not at all. In addition it might be advantageously to combine the nucleic acids sequences of the invention containing the sequences shown in the respective line in Table I, application no. 11, column 5 or 8, preferably the coding region thereof, or homologs or fragments thereof, with genes which generally support or enhance the growth or yield of the target non-human organisms, for example genes which lead to faster growth rate of non human organism like microorganisms or plants or genes which produce stress-, pathogen-, or herbicide-resistant plants.

[0227.1.8.11] to [0239.1.8.11] for the disclosure of these paragraphs see [0227.1.8.8] to [0239.1.8.8] above.

In an embodiment of the present invention the nucleic acid construct of the present invention comprises a nucleic acid molecule as depicted in the respective line in Table I, application no. 11, column 5 or 8, preferably the coding region thereof, a fragment or a homolog thereof, and a plant promoter, such as the Big 35S promoter, the DC3 promoter, the PC

Ubi promoter, the LegB4-promoter, the Super promoter, the USP promoter or the like. In another embodiment the nucleic acid construct of the present invention comprises a nucleic acid molecule as depicted in the respective line in Table I, application no. 11, column 5 or 8, preferably the coding region thereof, a fragment or a homolog thereof, and a plant promoter such as, for example, the USP, the LegB4-, the DC3 promoter or the ubiquitin promoter from parsley or other herein mentioned promoters. In a further embodiment the nucleic acid construct of the present invention comprises a nucleic acid molecule as depicted in the respective line in Table I, application no. 11, column 5 or 8, preferably the coding region thereof, a fragment or a homolog thereof, and a plant promoter, such as the Big 35S promoter, the PC Ubi promoter, the Super promoter, the USP promoter or the like. In addition different terminators may advantageously be used in these nucleic acid constructs; examples for transcriptional termination are polyadenylation signals.

[0241.1.8.11] to [0245.1.8.11] for the disclosure of this paragraph see [0241.1.8.8] to [0245.1.8.8] above.

Other preferred sequences for use in operable linkage in gene expression constructs are targeting sequences, which are required for targeting the gene product into specific cell compartments (for a review, see Kermode, Crit. Rev. Plant Sci. 15 (4), 285 (1996) and references cited therein), for example into the vacuole, the nucleus, all types of plastids, such as amyloplasts, chloroplasts, chromoplasts, the extracellular space, the mitochondria, the endoplasmic reticulum, elaioplasts, peroxisomes, glycosomes, and other compartments of cells or extracellular; preferred are sequences, which are involved in targeting to plastids as mentioned above. Sequences, which must be mentioned in this context are, in particular, the signal-peptideor transit-peptide-encoding sequences which are known per se. For example, plastidtransit-peptide-encoding sequences enable the targeting of the expression product into the plastids of a plant cell. Targeting sequences are also known for eukaryotic and to a lower extent for prokaryotic organisms and can advantageously be operable linked with the nucleic acid molecule of the present invention as shown in Table I, application no. 11, columns 5 and 8, preferably the coding region thereof, or homologs or fragments thereof, and described herein to achieve an expression in one of said compartments or extracellular.

[0247.1.8.11] to [0266.1.8.11] for the disclosure of these paragraphs see [0247.1.8.8] to [0266.1.8.8] above.

An embodiment of the invention relates to a vector where the nucleic acid molecule according to the invention is linked operably to regulatory sequences which permit the expression in a prokaryotic or eukaryotic host. A further embodiment of the invention relates to a vector in which a nucleic acid sequence encoding one of the polypeptides shown in the respective line in Table II, application no. 11, columns 5 or 8, or homologs or fragments thereof, is functionally linked to a targeting sequence such as a plastidial or mitochondrial targeting sequence. A further embodiment of the invention relates to a vector in which a nucleic acid sequence encoding one of the polypeptides shown in the respective line in Table II, application no. 11, columns 5 or 8, or homologs or fragments thereof, is functionally linked to (a) regulatory sequence which permit the expression in an organelle, such as plastids or mitochondria, preferably plastids.

A further embodiment of the invention relates to a vector which comprises a nucleic acid sequence encoding one of the polypeptides shown in the respective line in Table II, application no. 11, columns 5 or 8, or homologs or fragments thereof, but no additional targeting sequence.

[0268.1.8.11] to [0273.1.8.11] for the disclosure of these paragraphs see [0268.1.8.8] to [0273.1.8.8] above.

A further object of the invention relates to the use of a nucleic acid construct, e.g. an expression cassette, comprising one or more nucleic acid molecules encoding one or more polypeptides as depicted in the respective line(s) in Table II, application no. 11, columns 5 or 8, or homologs or fragments thereof, or comprising one or more nucleic acid molecules as depicted in the respective line(s) in Table I, application no. 11, columns 5 or 8, preferably the coding region thereof, or homologs or fragments thereof, or comprising a nucleic acid molecule hybridizing therewith for the transformation of a microorganism or a plant cell, plant tissues or a part of a plant.

In doing so, depending on the choice of promoter, the nucleic acid molecules shown in the respective line in Table I, application no. 11, column 5 or 8, preferably the coding region thereof, or homologs or fragments thereof, or the nucleic acid molecules encoding a polypeptide as depicted in the respective line in Table II, application no. 11, column 5 or 8, or homologs or fragments thereof, can be expressed in the whole plant or specifically e.g. in the leaves, in the seeds, the nodules, in roots, in the stem or other parts of the plant. Those transgenic plants overproducing sequences, e.g. as depicted in the respective line in Table I, application no. 11, column 5 or 8, preferably the coding region thereof, or homologs or fragments thereof, the plant cell, plant tissues, plants, the reproductive material thereof, or parts thereof are a further object of the present invention.

Within the framework of the present invention, the production of the palmitic acid is due to the generation or over-expression of one or more polypeptides as depicted in the respective line(s) in Table II, application no. 11, column 5 or 8, or homologs or fragments thereof, or encoded by the corresponding nucleic acid molecules as depicted in the respective line(s) in Table I, application no. 11, column 5 or 8, preferably the coding region thereof, or homologs or fragments thereof, in the non-human organism according to the invention, advantageously in the transgenic microorganism or plant according to the invention, at least for the duration of at least one plant generation.

A constitutive expression of the polypeptide as depicted in the respective line in Table II, application no. 11, column 5 or 8, or homologs or fragments thereof, or encoded by the respective nucleic acid molecule as depicted in the respective line in Table I, application no. 11, column 5 or 8, preferably the coding region thereof, or homologs or fragments thereof, may be advantageous. On the other hand, an inducible expression may also appear desirable. Expression of the polypeptide sequences of the invention can be either directed to the cytosol or to the organelles, such as plastids or mitochondria, preferably the plastids of the host cells, preferably the plant cells, or non-targeted.

for the disclosure of this paragraph see [0278.1.8.8] above.

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 11, column 5 or 8, preferably shown in Table II A, application no. 11, column 5, or in Table II A, application no. 11, column 8, or in Table II B, application no. 11, column 8, or a homolog or a fragment thereof;
   (b) a nucleic acid molecule shown in Table I, application no. 11, column 5 or 8, preferably shown in Table I A, application no. 11, column 5, or in Table I A, application no. 11, column 8, or in Table I B, application no. 11, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 11, preferably shown in Table II A, application no. 11, column 5, or in Table II A, application no. 11, column 8, or in Table II B, application no. 11, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 11, preferably shown in Table I A, application no. 11, column 5, or in Table I A, application no. 11, column 8, or in Table I B, application no. 11, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 11;
   (i) a nucleic acid molecule encoding a polypeptide having the activity represented by a respective protein as depicted in column 5 of Table II, application no. 11;
   (j) a nucleic acid molecule comprising a polynucleotide which is obtained by amplifying a cDNA library or a genomic library using the primers in Table III, application no. 11, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising an expression cassette comprising
[0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 11, column 5 or 8, preferably shown in Table II A, application no. 11, column 5, or in Table II A, application no. 11, column 8, or in Table II B, application no. 11, column 8, or a homolog or a fragment thereof;
   (b) a nucleic acid molecule shown in Table I, application no. 11, column 5 or 8, preferably shown in Table I A, application no. 11, column 5, or in Table I A, application no. 11, column 8, or in Table I B, application no. 11, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 11, preferably shown in Table II A, application no. 11, column 5, or in Table II A, application no. 11, column 8, or in Table II B, application no. 11, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 11, preferably shown in Table I A, application no. 11, column 5, or in Table I A, application no. 11, column 8, or in Table I B, application no. 11, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 11;
   (i) a nucleic acid molecule encoding a polypeptide having the activity represented by a respective protein as depicted in column 5 of Table II, application no. 11;
   (j) a nucleic acid molecule comprising a polynucleotide which is obtained by amplifying a cDNA library or a genomic library using the primers in Table III, application no. 11, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising or being transformed by a vector comprising
[1) a nucleic acid molecule selected from the group consisting of
   (a) a nucleic acid molecule encoding a polypeptide shown in Table II, application no. 11, column 5 or 8, preferably shown in Table II A, application no. 11, column 5, or in Table II A, application no. 11, column 8, or in Table II B, application no. 11, column 8, or a homolog or a fragment thereof;
   (b) a nucleic acid molecule shown in Table I, application no. 11, column 5 or 8, preferably shown in Table I A, application no. 11, column 5, or in Table I A, application no. 11, column 8, or in Table I B, application no. 11, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 11, preferably shown in Table II A, application no. 11, column 5, or in Table II A, application no. 11, column 8, or in Table II B, application no. 11, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 11, preferably shown in Table I A, application no. 11, column 5, or in Table I A, application no. 11, column 8, or in Table I B, application no. 11, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 11;
   (i) a nucleic acid molecule encoding a polypeptide having the activity represented by a respective protein as depicted in column 5 of Table II, application no. 11;
   (j) a nucleic acid molecule comprising a polynucleotide which is obtained by amplifying a cDNA library or a genomic library using the primers in Table III, application no. 11, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 11.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 11 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 11, in the same line as of the respective nucleic acid molecule according to 1) “plastidic” is depicted, or encoding a mitochondrial transit peptide, especially in case in column 6 of Table I, application no. 11, in the same line as of the respective nucleic acid molecule according to 1) “mitochondrial” is depicted.

In another embodiment of said transgenic non-human organism the expression cassette does not comprise 2) a nucleic acid molecule encoding a transit peptide, especially in case in column 6 of Table I, application no. 11, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 11.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 11, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 11, in the same line as of the respective nucleic acid molecule according to 1) “plastidic” is depicted, or encoding a mitochondrial transit peptide, especially in case in column 6 of Table I, application no. 11, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 11, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 11, in the same line as of the respective nucleic acid molecule according to 1) “plastidic” is depicted, or encoding a mitochondrial transit peptide, especially in case in column 6 of Table I, application no. 11, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 11, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 11, in the same line as of the respective nucleic acid molecule according to 1) “plastidic” is depicted, or encoding a mitochondrial transit peptide, especially in case in column 6 of Table I, application no. 11, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 11.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 11, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 11.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 11, in the same line as of the respective nucleic acid molecule according to 1) “plastidic” is depicted, or encoding a mitochondrial transit peptide, especially in case in column 6 of Table I, application no. 11, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 11, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 11, in the same line as of the respective nucleic acid molecule according to 1) “plastidic” is depicted, or encoding a mitochondrial transit peptide, especially in case in column 6 of Table I, application no. 11, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 11 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 11, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 11 (in the same line) and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 11, column 5 or 8, preferably shown in Table II A, application no. 11, column 5, or in Table II A, application no. 11, column 8, or in Table II B, application no. 11, column 8, or a homolog or a fragment thereof;
   (b) a nucleic acid molecule shown in Table I, application no. 11, column 5 or 8, preferably shown in Table I A, application no. 11, column 5, or in Table I A, application no. 11, column 8, or in Table I B, application no. 11, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 11, preferably shown in Table II A, application no. 11, column 5, or in Table II A, application no. 11, column 8, or in Table II B, application no. 11, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 11, preferably shown in Table I A, application no. 11, column 5, or in Table I A, application no. 11, column 8, or in Table I B, application no. 11, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 11;
   (i) a nucleic acid molecule encoding a polypeptide having the activity represented by a respective protein as depicted in column 5 of Table II, application no. 11;
   (j) a nucleic acid molecule comprising a polynucleotide which is obtained by amplifying a cDNA library or a genomic library using the primers in Table III, application no. 11, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising an expression cassette comprising
[0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 11, column 5 or 8, preferably shown in Table II A, application no. 11, column 5, or in Table II A, application no. 11, column 8, or in Table II B, application no. 11, column 8, or a homolog or a fragment thereof;
   (b) a nucleic acid molecule shown in Table I, application no. 11, column 5 or 8, preferably shown in Table I A, application no. 11, column 5, or in Table I A, application no. 11, column 8, or in Table I B, application no. 11, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 11, preferably shown in Table II A, application no. 11, column 5, or in Table II A, application no. 11, column 8, or in Table II B, application no. 11, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 11, preferably shown in Table I A, application no. 11, column 5, or in Table I A, application no. 11, column 8, or in Table I B, application no. 11, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 11;
   (i) a nucleic acid molecule encoding a polypeptide having the activity represented by a respective protein as depicted in column 5 of Table II, application no. 11;
   (j) a nucleic acid molecule comprising a polynucleotide which is obtained by amplifying a cDNA library or a genomic library using the primers in Table III, application no. 11, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      and, if desired
2) nucleic acid molecule encoding a transit peptide, preferably encoding a plastidal transit peptide or a mitochondrial transit peptide;
   which are operable linked;]
   or comprising or being transformed by a vector comprising
[1) a nucleic acid molecule selected from the group consisting of
   (a) a nucleic acid molecule encoding a polypeptide shown in Table II, application no. 11, column 5 or 8, preferably shown in Table II A, application no. 11, column 5, or in Table II A, application no. 11, column 8, or in Table II B, application no. 11, column 8, or a homolog or a fragment thereof;
   (b) a nucleic acid molecule shown in Table I, application no. 11, column 5 or 8, preferably shown in Table I A, application no. 11, column 5, or in Table I A, application no. 11, column 8, or in Table I B, application no. 11, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 11, preferably shown in Table II A, application no. 11, column 5, or in Table II A, application no. 11, column 8, or in Table II B, application no. 11, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 11, preferably shown in Table I A, application no. 11, column 5, or in Table I A, application no. 11, column 8, or in Table I B, application no. 11, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 11;
   (i) a nucleic acid molecule encoding a polypeptide having the activity represented by a respective protein as depicted in column 5 of Table II, application no. 11;
   (j) a nucleic acid molecule comprising a polynucleotide which is obtained by amplifying a cDNA library or a genomic library using the primers in Table III, application no. 11, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      whereby, preferably, the nucleic acid molecule according to 1 (a), (b), (c), (d), (e), (f), (g), (h), (i), (j) or (k) distinguishes over the sequence depicted in Table I A and/or I B, application no. 11, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 11, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 11, column 5 or 8, or the coding region thereof, respectively. In a further embodiment the nucleic acid molecule does not encode the polypeptide sequence shown in Table II A and/or II B, application no. 11, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 11, column 5 or 8 and does not encode a protein of the sequence shown in Table II A and/or II B, application no. 11, column 5 or 8. Accordingly, in one embodiment, the protein encoded by a sequence of a nucleic acid according to 1 (a), (b), (c), (d), (e), (f), (g), (h), (i), (j) or (k) does not consist of the sequence shown in Table I A and/or I B, application no. 11, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 11, column 5 or 8 but less than 100%, preferably less than 99.999%, 99.99%, 99.9%, 99%, 98%, 97%, 96% or 95% identical to the sequence shown in Table II A and/or II B, application no. 11, column 5 or 8;
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 11.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 11 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 11, in the same line as of the respective nucleic acid molecule according to 1) “plastidic” is depicted, or encoding a mitochondrial transit peptide, especially in case in column 6 of Table I, application no. 11, in the same line as of the respective nucleic acid molecule according to 1) “mitochondrial” is depicted.

In another embodiment thereof the expression cassette does not comprise 2) a nucleic acid molecule encoding a transit peptide, especially in case in column 6 of Table I, application no. 11, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 11.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the same line in column 5 of Table II, application no. 11, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 11, in the same line as of the respective nucleic acid molecule according to 1) “plastidic” is depicted, or encoding a mitochondrial transit peptide, especially in case in column 6 of Table I, application no. 11, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 11, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 11, in the same line as of the respective nucleic acid molecule according to 1) “plastidic” is depicted, or encoding a mitochondrial transit peptide, especially in case in column 6 of Table I, application no. 11, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 11, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 11, in the same line as of the respective nucleic acid molecule according to 1) “plastidic” is depicted, or encoding a mitochondrial transit peptide, especially in case in column 6 of Table I, application no. 11, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 11.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 11, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 11.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 11, in the same line as of the respective nucleic acid molecule according to 1) “plastidic” is depicted, or encoding a mitochondrial transit peptide, especially in case in column 6 of Table I, application no. 11, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 11, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 11, in the same line as of the respective nucleic acid molecule according to 1) “plastidic” is depicted, or encoding a mitochondrial transit peptide, especially in case in column 6 of Table I, application no. 11, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 11 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 11, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 11 and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

[0291.1.8.11] to [0299.1.8.11] for the disclosure of these paragraphs see [0291.1.8.8] to [0299.1.8.8] above.

The term “biological active portion” or “biological activity” means a polypeptide as depicted in the respective line in Table II, application no. 11, column 3 or a portion of said polypeptide which still has at least 10% or 20%, preferably 30%, 40%, 50% or 60%, especially preferably 70%, 75%, 80%, 90% or 95% of the enzymatic or biological activity of the natural or starting enzyme or protein.

[0301.1.8.11] to [0304.1.8.11] for the disclosure of these paragraphs see [0301.1.8.8] to [0304.1.8.8] above.

Synthetic oligonucleotide primers for the amplification, e.g. as shown in the respective line in Table III, application no. 11, column 8, by means of polymerase chain reaction can be generated on the basis of a sequence shown herein, for example the sequence shown in the respective line in Table I, application no. 11, columns 5 or 8, or the sequences derived from Table II, application no. 11, columns 5 or 8, respectively.

Moreover, it is possible to identify a conserved region of a protein from various organism by carrying out protein sequence alignments with the polypeptide encoded by the nucleic acid molecules of the present invention, in particular with the sequences encoded by the nucleic acid molecule shown in the respective line in column 5 or 8 of Table I, application no. 11, from which conserved regions, and in turn, degenerate primers can be derived. Conserved regions are those, which show a very little variation in the amino acid in one particular position of several homologs from different origin. The consensus sequence and polypeptide motifs shown in the respective line in column 8 of Table IV, application no. 11, are derived from such alignments of homologous proteins.

In an embodiment of the present invention, in the method of the present invention the activity of a polypeptide comprising or consisting of a consensus sequence or at least one polypeptide motif shown in the respective line in Table IV, application no. 11, column 8, is increased. In another embodiment thereof, the method of the present invention relates to a polypeptide comprising or consisting of a consensus sequence shown in the respective line in Table IV, application no. 11, column 8 or comprising at least one polypeptide motif shown in the respective line in Table IV, application no. 11, column 8, whereby not more than 20, 15, 10, 9, 8, 7, 6, 5 4, 3, 2 or 1, or 0 of the amino acid positions indicated can be replaced by any amino acid. In another embodiment thereof, the method of the present invention relates to a polypeptide comprising or consisting of a consensus sequence shown in the respective line in Table IV, application no. 11, column 8 or comprising at least one polypeptide motif shown in the respective line in Table IV, application no. 11, column 8, whereby not more than 15%, 10%, 5%, 4%, 3%, 2% or 1%, or 0% of the amino acid positions defined as distinct specific amino acids are/is replaced by another amino acid. In another embodiment thereof, the method of the present invention relates to a polypeptide comprising or consisting of a consensus sequence shown in the respective line in Table IV, application no. 11, column 8 or comprising at least one polypeptide motif shown in the respective line in Table IV, application no. 11, column 8, whereby not more than 20, 15, 10, 9, 8, 7, 6, 5, 4, 3, 2 or 1, or 0 amino acids are inserted into a consensus sequence or polypeptide motif.

In an embodiment of the present invention, in the method of the present invention the activity of a polypeptide comprising or consisting of a consensus sequence or at least one polypeptide motif shown in the respective line in Table IV, application no. 11, column 8, is increased, whereby said polypeptide distinguishes over the sequence depicted in Table II, application no. 11, columns 5 or 8 by one or more amino acids.

In one embodiment, said polypeptide of the invention distinguishes over the sequence shown in the respective line in Table II, application no. 11, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II B, application no. 11, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in Table II, application no. 11, columns 5 or 8.

[0309.1.8.11] to [0321.1.8.11] for the disclosure of these paragraphs see [0309.1.8.8] to [0321.1.8.8] above.

Polypeptides having above-mentioned activity, i.e. conferring the production or the increased production of the fine chemical palmitic acid as compared to a corresponding, e.g. non-transformed, wild type non-human organism, like a microorganism or a plant cell, plant or part thereof, can be encoded by other DNA sequences which hybridize to the sequences shown in the respective line in Table I, application no. 11, columns 5 and 8, preferably the coding region thereof, at least under relaxed hybridization conditions and which encode the expression of polypeptides conferring the production or the increased production of the respective fine chemical palmitic acid as compared to a corresponding, e.g. non-transformed, wild type non-human organism, like a microorganism or a plant cell, plant or part thereof.

[0323.1.8.11] to for the disclosure of these paragraphs see [0323.1.8.8] to [0329.1.8.8] above [Further, the nucleic acid molecule of the invention comprises a nucleic acid molecule, which is a complement of one of the nucleotide sequences of above-mentioned nucleic acid molecules or a portion thereof. A nucleic acid molecule or its sequence which is complementary to one of the nucleotide molecules or sequences shown in the respective line in Table I, application no. 11, columns 5 or 8, preferably the coding region thereof, homologs or fragments thereof, is one which is sufficiently complementary to one of the nucleotide molecules or sequences shown in the respective line in Table I, application no. 11, columns 5 or 8, preferably the coding region thereof, homologs or fragments thereof, such that it can hybridize to one of the nucleotide sequences shown in Table I, application no. 11, columns 5 or 8, preferably the coding region thereof, homologs or fragments thereof, thereby forming a stable duplex. Preferably, the hybridization is performed under stringent hybridization conditions. However, a complement of one of the herein disclosed sequences is preferably a sequence complement thereto according to the base pairing of nucleic acid molecules well known to the skilled person. For example, the bases A and G undergo base pairing with the bases T and U or C, resp. and visa versa. Modifications of the bases can influence the base-pairing partner.

The nucleic acid molecule of the invention comprises a nucleotide sequence which is at least about 30%, 35%, 40%, 45%, 50%, 55%, 60%, 65%, 70%, 75%, 80%, 90%, 95%, 96%, 97%, 98%, 99%, 99.5% or more homologous to a nucleotide sequence shown in Table I, application no. 11, columns 5 or 8, preferably the coding region thereof, homologs or fragments thereof, and preferably has above-mentioned activity, in particular conferring the production or the increased production of the fine chemical palmitic acid, respectively, after increasing the activity or an activity of a gene as shown in the respective line in Table I or of a gene product, e.g. as shown in the respective line in Table II, application no. 11, column 5 or 8, by for example in one embodiment expression either in the cytosol or in an organelle such as a plastid or mitochondria or both, preferably in a plastid, or in another embodiment non-targeted or targeted.

The nucleic acid molecule of the invention comprises a nucleotide sequence or molecule which hybridizes, preferably hybridizes under stringent conditions as defined herein, to one of the nucleic acid molecule shown in the respective line in Table I, preferably Table IB, application no. 11, columns 5 or 8, preferably the coding region thereof, fragments or homologs thereof, and encodes a protein having above-mentioned activity, e.g. conferring the production or the increased production of the respective fine chemical palmitic acid as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted, and optionally, the activity thereof is selected from the group consisting of 2-C-methyl-D-erythritol 4-phosphate cytidylyltransferase, 3-ketoacyl-CoA thiolase, 49747384_SOYBEAN-protein, AAS bifunctional protein, ABC metal ion transporter substrate-binding protein, acid phosphatase precursor, acid shock protein, aconitate hydratase, acyl transferase, arginine decarboxylase, aromatic acid decarboxylase, At1g19800-protein, At1g23110-protein, At2g45420-protein, ATPase epsilon subunit, b0050-protein, b1234-protein, b1248-protein, b1330-protein, b1670-protein, b1837-protein, b2032-protein, b2099-protein, b2107-protein, b2399-protein, b2682-protein, b2849-protein, b3814-protein, bifunctional protein (phosphoribosyltransferase and regulatory protein), branched chain amino acid ABC transporter ATP-binding protein, calcium-dependent protein kinase, carbamoyl-phosphate synthase subunit, carbon dioxide concentrating mechanism protein, cation-transporting ATPase, CCAAT-binding transcription factor, chlorophyllase, colanic acid biosynthesis protein, coproporphyrinogen III oxidase, DnaJ-like chaperone, ethanolamine utilization protein, F-box protein, Fe—S subunit of oxidoreductase, flavodoxin, glucose-6-phosphate 1-dehydrogenase, glutaredoxin, glutathione S-transferase, glycerol-3-phosphate dehydrogenase, heat shock transcription factor, HesB/YadR/YfhF family protein, homocitrate synthase, lipoprotein precursor, metal-dependent hydrolase, monothiol glutaredoxin, NADH-quinone oxidoreductase subunit, nitrate/nitrite transport protein, oxidoreductase, peptidyl-prolyl cis-trans isomerase, permease protein of ABC transporter, phosphate import ATP-binding protein, phosphoadenosine phosphosulfate reductase, phosphoribosylformyl glycinamidine synthase subunit, protein kinase, protein phosphatase, purine nucleoside phosphorylase, recombinase A, sll1032-protein, sodium/proton antiporter, ThiF family protein, threonine dehydratase, threonine efflux protein, transcription factor, and ymr013c-protein, respectively.

Moreover, the nucleic acid molecule of the invention can comprise only a portion of the coding region of one of the nucleic acid molecules depicted in Table I, application no. 11, columns 5 or 8, for example a fragment which can be used as a probe or primer or a fragment encoding a biologically active portion of the polypeptide of the present invention or of a polypeptide used in the process of the present invention, i.e. having above-mentioned activity, e.g. conferring the production or the increased production of the respective fine chemical palmitic acid as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted. The nucleotide sequences determined from the cloning of the present protein-according-to-the-inventionencoding gene allows for the generation of probes and primers designed for use in identifying and/or cloning its homologues from other cell types and organisms. The probe/primer typically comprises a substantially purified oligonucleotide. The oligonucleotide typically comprises a region of nucleotide sequence that hybridizes under stringent conditions to at least about 12, 15 preferably about 20 or 25, more preferably about 40, 50 or 75 consecutive nucleotides of a sense strand of one of the sequences set forth, e.g., in the respective line in Table I, application no. 11, column 5 or 8, preferably the coding region thereof, or a homolog or a fragment thereof, an anti-sense sequence of one of the sequences, e.g., set forth in the respective line in Table I, application no. 11, columns 5 or 8, preferably the coding region thereof, or a homolog or a fragment thereof or naturally occurring mutants thereof. Primers based on a nucleic acid molecule of the invention can be used in PCR reactions to clone homologs of the polypeptide of the invention or of the polypeptide used in the process of the invention, e.g. as the primers described in the examples of the present invention, e.g. as shown in the examples. A PCR with the primers shown in the respective line in Table III, column 8 will result in a fragment of the gene product as shown in Table II, application no. 11, column 5.

Primer sets are interchangeable. The person skilled in the art knows how to design and combine said primers to result in the desired product, e.g. in a full length clone or a partial sequence. Probes based on the sequences of the nucleic acid molecule of the invention or used in the process of the present invention can be used to detect transcripts or genomic sequences encoding the same or homologous proteins. The probe can further comprise a label group attached thereto, e.g. the label group can be a radioisotope, a fluorescent compound, an enzyme, or an enzyme co-factor. Such probes can be used as a part of a genomic marker test kit for identifying cells which express a polypeptide of the invention or used in the process of the present invention, such as by measuring a level of an encoding nucleic acid molecule in a sample of cells, e.g. detecting mRNA levels or determining, whether a genomic gene comprising the sequence of the polynucleotide of the invention or used in the processes of the present invention has been mutated or deleted.

As the expression of a respective nucleic acids of the invention is related to the synthesis of the respective fine chemical palmitic acid its function as a probe extends to the detection of microorganisms, plant tissues, plants, plant variets, plant ecotypes or plant genera with varying capability or potential for synthesis of the respective fine chemical palmitic acid. Therefore in one embodiment the present invention relates to a method for analyzing the capability or potential of a plant tissue, a plant, a plant variety or plant ecotype to produce the fine chemical palmitic acid by using the nucleic acid of the invention or parts thereof as a probe to detect the amount of the nucleic acid of the invention in the non-human organism or a part thereof in comparison to another non-human organism.

The nucleic acid molecule of the invention encodes a polypeptide or portion thereof which includes an amino acid sequence which is sufficiently homologous to the amino acid sequence shown in respective line in Table II, application no. 11, columns 5 or 8 such that the protein or portion thereof maintains the ability to confer the production or the increased production of the respective fine chemical palmitic acid as compared to a corresponding, e.g. non-transformed, wild type non-human organism, like a microorganism or a plant cell, plant or part thereof, in particular increasing the activity as mentioned above or as described in the examples in microorganisms or plants.

As used herein, the language “sufficiently homologous” refers to proteins or portions thereof which have amino acid sequences which include a minimum number of identical or equivalent amino acids (e.g. an amino acid which has a similar side chain as an amino acid in one of the sequences of the polypeptide of the present invention) to an amino acid sequence shown in the respective line in Table II, application no. 11, columns 5 or 8 such that the protein or portion thereof is able to confer the production or the increased production of the respective fine chemical palmitic acid as compared to a corresponding, e.g. non-transformed, wild type non-human organism, like a microorganism or a plant cell, plant or part thereof. For example having the activity of a protein as shown in the respective line in Table II, application no. 11, column 3 and as described herein.

In an embodiment of the present invention, the nucleic acid molecule of the present invention comprises a nucleic acid molecule that encodes a portion of the protein of the present invention. The protein is at least about 30%, 35%, 40%, 45% 50%, 55%, 60%, 65%, 70%, 75%, 80%, 85%, 90%, 91%, 92%, 93%, 94%, 95%, 96%, 97%, 98%, 99%, 99.5% or more homologous to an entire amino acid sequence shown in the respective line of Table II, application no. 11, column 5 or 8 and has the above-mentioned activity, e.g. conferring the production or the increased production of the respective fine chemical palmitic acid as compared to a corresponding, e.g. non-transformed, wild type non-human organism, like a microorganism or a plant cell, plant or part thereof by, for example in a embodiment expression either in the cytosol or in an organelle such as a plastid or mitochondria or both, preferably in a plastid, or in another embodiment by targeted or non-targeted expression.

[0338.1.8.11] to [0339.1.8.11] for the disclosure of these paragraphs see [0338.1.8.8] to [0339.1.8.8] above.

The invention further relates to nucleic acid molecules or methods using said nucleic acid molecules that differ from one of the nucleotide sequences shown in the respective line in Table I, application no. 11, columns 5 or 8, preferably the coding region thereof, or fragments or homologs thereof, due to degeneracy of the genetic code but encode a polypeptide of the present invention, in particular a polypeptide having the above-mentioned activity, e.g. as those polypeptides depicted in the respective line in Table II, application no. 11, columns 5 or 8, fragments or homologs thereof. Advantageously, the nucleic acid molecule of the invention comprises, or in an other embodiment has, a nucleotide sequence encoding a protein comprising, or in an other embodiment having, an amino acid sequence shown in the respective line in Table II, application no. 11, columns 5 or 8, fragments or homologs thereof. In a further embodiment, the nucleic acid molecule of the invention encodes a full length protein which is substantially homologous to an amino acid sequence shown in the respective line in Table II, application no. 11, columns 5 or 8, or homologs thereof. However, in an embodiment, the nucleic acid molecule of the present invention does not consist of the sequence shown in the respective line in Table I, preferably Table IA, application no. 11, columns 5 or 8, preferably the coding region thereof, but the functional homologs thereof.

[0341.1.8.11] to [0343.1.8.11] for the disclosure of these paragraphs see [0341.1.8.8] to [0343.1.8.8] above.

Accordingly, in another embodiment, a nucleic acid molecule of the invention is at least 15, 20, 25 or 30 nucleotides in length. Preferably, it hybridizes under stringent conditions to a nucleic acid molecule comprising a nucleotide sequence of the nucleic acid molecule of the present invention or used in the process of the present invention, e.g. comprising the sequence shown in the respective line in Table I, application no. 11, columns 5 or 8, preferably the coding region thereof. In another embodiment the nucleic acid molecule is preferably at least 20, 30, 50, 100, 250 or more nucleotides in length.

for the disclosure of this paragraph see [0345.1.8.8] above.

Preferably, a nucleic acid molecule of the invention that hybridizes under stringent conditions to a sequence shown in the respective line in Table I, application no. 11, columns 5 or 8, preferably the coding region thereof, corresponds to a naturally-occurring nucleic acid molecule of the invention. As used herein, a “naturally-occurring” nucleic acid molecule refers to a RNA or DNA molecule having a nucleotide sequence that occurs in nature (e.g. encodes a natural protein). Preferably, the nucleic acid molecule encodes a natural protein having above-mentioned activity, e.g. conferring the production or the increased production of the respective fine chemical palmitic acid as compared to a corresponding, e.g. non-transformed, wild type non-human organism, like a microorganism or a plant cell, plant or part thereof after increasing the expression or activity thereof or the activity of a protein of the invention or used in the process of the invention, in an embodiment for example expression either in the cytosol or in an organelle such as a plastid or mitochondria, preferably in plastids, or, in another embodiment by targeted or non-targeted expression.

for the disclosure of this paragraph see [0347.1.8.8] above.

For example, nucleotide substitutions leading to amino acid substitutions at “non-essential” amino acid residues can be made in a sequence of the nucleic acid molecule of the invention or used in the process of the invention, e.g. shown in the respective line in Table I, application no. 11, columns 5 or 8, preferably the coding region thereof, or fragments or homologs thereof.

[0349.1.8.11] to [0350.1.8.11] for the disclosure of these paragraphs see [0349.1.8.8] to [0350.1.8.8] above.

Accordingly, the invention relates to nucleic acid molecules encoding a polypeptide having above-mentioned activity, in a non-human organisms or parts thereof by for example expression either in the cytosol or in an organelle such as a plastid or mitochondria or both, preferably in plastids, that contain changes in amino acid residues that are not essential for said activity. Such polypeptides differ in amino acid sequence from a sequence depicted in the respective line in Table II, especially Table IIA, application no. 11, columns 5 or 8, but retain said activity described herein. The nucleic acid molecule can comprise a nucleotide sequence encoding a polypeptide, wherein the polypeptide comprises an amino acid sequence at least about 50% identical to an amino acid sequence shown in the respective line in Table II, application no. 11, columns 5 or 8, and is capable of conferring the production or the increased production of the respective fine chemical palmitic acid as compared to a corresponding, e.g. non-transformed, wild type non-human organism, like a microorganism or a plant cell, plant or part thereof, after increasing its activity for example in an embodiment by expression either in the cytosol or in an organelle such as a plastid or mitochondria or both, preferably in plastids, or, in another embodiment by targeted or non-targeted expression. Preferably, the protein encoded by the nucleic acid molecule is at least about 60%, 70%, 80%, 90%, 95%, 96%, 97%, 98%, 99% or 99.5% identical to the sequence shown in the respective line in Table II, application no. 11, columns 5 or 8.

[0352.1.8.11] to [0357.1.8.11] for the disclosure of these paragraphs see [0352.1.8.8] to [0357.1.8.8] above.

Functional equivalents of the nucleic acid molecules according to the present invention and/or used in the process according to the present invention represent an embodiment of homologs. Functional equivalents derived from the nucleic acid molecules as shown in the respective line in Table I, application no. 11, columns 5 or 8, preferably the coding region thereof, according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the nucleic acid molecules as shown in the respective line in Table I, application no. 11, columns 5 or 8, preferably the coding region thereof, according to the invention and encode polypeptides having essentially the same properties as the polypeptide as shown in the respective line in Table II, application no. 11, columns 5 or 8.

Functional equivalents derived from one of the polypeptides as shown in the respective line in Table II, application no. 11, columns 5 or 8 according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the polypeptides as shown in the respective line in Table II, application no. 11, columns 5 or 8 according to the invention and having essentially the same properties as the polypeptide as shown in the respective line in Table II, application no. 11, columns 5 or 8.

for the disclosure of this paragraph see [0359.1.8.8] above.

A nucleic acid molecule encoding a homolog to a polypeptide depicted in the respective line in Table II, application no. 11, columns 5 or 8 can be created by introducing one or more nucleotide substitutions, additions or deletions into the nucleic acid molecule of the present invention, in particular of Table I, application no. 11, columns 5 or 8, in the respective line, such that one or more amino acid substitutions, additions or deletions are introduced into the encoded protein. Mutations can be introduced into the nucleic acid molecules as depicted in the respective line in Table I, application no. 11, columns 5 or 8, preferably the coding region thereof, by standard techniques, such as site-directed mutagenesis, PCR-mediated mutagenesis or other methods known to the person skilled in the art.

[0361.1.8.11] to [0363.1.8.11] for the disclosure of these paragraphs see [0361.1.8.8] to [0363.1.8.8] above.

Homologues of the nucleic acid sequences used, with the sequences shown in the respective line in Table I, application no. 11, columns 5 or 8, preferably the coding region thereof, comprise also allelic variants with at least approximately 30%, 35%, 40%, 45%, 50%, 55%, 60%, 65%, 70%, 75%, 80%, 85%, 90%, 91%, 92%, 93%, 94% 95%. 96%, 97%, 98%, 99%, 99.5% or more % homology with one of the nucleotide sequences shown or the abovementioned derived nucleic acid sequences or their homologues or derivatives or parts of these. Allelic variants encompass in particular functional variants which can be obtained by deletion, insertion or substitution of nucleotides from the sequences shown in the respective line, preferably in Table I, columns 5 or 8, preferably the coding region thereof, or from the derived nuoleic acid sequences, the intention being, however, that the enzymatic activity or the biological activity of the resulting proteins synthesized is advantageously retained or increased.

In one embodiment of the present invention, the nucleic acid molecule of the invention or used in the process of the invention comprises the sequences shown in the respective line in any of the Table I, application no. 11, columns 5 or 8, preferably the coding region thereof, fragments or homologs thereof. It is preferred that the nucleic acid molecule comprises as little as possible other nucleotides not shown in the respective line in any one of Table I, application no. 11, columns 5 or 8, preferably the coding region thereof, fragments or homologs thereof. In one embodiment, the nucleic acid molecule comprises less than 500, 400, 300, 200, 100, 90, 80, 70, 60, 50 or 40 further nucleotides. In a further embodiment, the nucleic acid molecule comprises less than 30, 20 or 10 further nucleotides. In one embodiment, the nucleic acid molecule used in the process of the invention is identical to the sequences shown in the respective line in Table I, application no. 11, columns 5 or 8, preferably the coding region thereof.

Also preferred is that the nucleic acid molecule used in the process of the invention encodes a polypeptide comprising the sequence shown in the respective line in Table II, application no. 11, columns 5 or 8, fragments or homologs thereof. In one embodiment, the nucleic acid molecule encodes less than 150, 130, 100, 80, 60, 50, 40 or 30 further amino acids. In a further embodiment, the encoded polypeptide comprises less than 20, 15, 10, 9, 8, 7, 6 or 5 further amino acids. In one embodiment used in the inventive process, the encoded polypeptide is identical to the sequences shown in the respective line in Table II, application no. 11, columns 5 or 8.

In one embodiment, the nucleic acid molecule of the invention or used in the process encodes a polypeptide comprising the sequence shown in the respective line in Table II, application no. 11, columns 5 or 8, or fragments or homologs thereof, and comprises less than 100 further nucleotides. In a further embodiment, said nucleic acid molecule comprises less than 30 further nucleotides. In one embodiment, the nucleic acid molecule used in the process is identical to a coding sequence of the sequences shown in the respective line in Table I, application no. 11, columns 5 or 8.

Polypeptides (=proteins), which still have the essential biological or enzymatic activity of the polypeptide of the present invention conferring the production or the increased production of the respective fine chemical palmitic acid as compared to a corresponding, e.g. non-transformed, wild type non-human organism, like a microorganism or a plant cell, plant or part thereof, i.e. whose activity is essentially not reduced, are polypeptides with at least 10%, 20%, 30%, 40%, 50%, 60%, 70%, 80%, 90%, 95% or more of the wild type biological activity or enzymatic activity, advantageously, the activity is essentially not reduced in comparison with the activity of a polypeptide shown in the respective line in Table II, application no. 11, columns 5 or 8 expressed under identical conditions.

Homologues of nucleic acid molecules shown in the respective line in Table I, application no. 11, columns 5 or 8, preferably the coding region thereof, or fragments thereof, or of the derived sequences shown in the respective line in Table II, application no. 11, columns 5 or 8, or fragments thereof, also mean truncated sequences, cDNA, single-stranded DNA or RNA of the coding and noncoding DNA sequence. Homologues of said sequences are also understood as meaning derivatives, which comprise noncoding regions such as, for example, UTRs, introns, terminators, enhancers or promoter variants. The promoters upstream of the nucleotide sequences stated can be modified by one or more nucleotide substitution(s), insertion(s) and/or deletion(s) without, however, interfering with the functionality or activity either of the promoters, the open reading frame (=ORF) or with the 3′-regulatory region such as terminators or other 3′-regulatory regions, which are far away from the ORF. It is furthermore possible that the activity of the promoters is increased by modification of their sequence, or that they are replaced completely by more active promoters, even promoters from heterologous organisms. Appropriate promoters are known to the person skilled in the art and are mentioned herein below.

[0370.1.8.11] to [0379.1.8.11] for the disclosure of these paragraphs see [0370.1.8.8] to [0379.1.8.8] above.

Moreover, a native polypeptide conferring the increase of the respective fine chemical palmitic acid in a non-human organism or a part thereof can be isolated from cells (e.g., endothelial cells), for example using the antibody of the present invention as described below, in particular, an antibody against proteins having 2-C-methyl-D-erythritol 4-phosphate cytidylyltransferase, 3-ketoacyl-CoA thiolase, 49747384_SOYBEAN-protein, AAS bifunctional protein, ABC metal ion transporter substrate-binding protein, acid phosphatase precursor, acid shock protein, aconitate hydratase, acyl transferase, arginine decarboxylase, aromatic acid decarboxylase, At1g19800-protein, At1g23110-protein, At2g45420-protein, ATPase epsilon subunit, b0050-protein, b1234-protein, b1248-protein, b1330-protein, b1670-protein, b1837-protein, b2032-protein, b2099-protein, b2107-protein, b2399-protein, b2682-protein, b2849-protein, b3814-protein, bifunctional protein (phosphoribosyltransferase and regulatory protein), branched chain amino acid ABC transporter ATP-binding protein, calcium-dependent protein kinase, carbamoyl-phosphate synthase subunit, carbon dioxide concentrating mechanism protein, cation-transporting ATPase, CCAAT-binding transcription factor, chlorophyllase, colanic acid biosynthesis protein, coproporphyrinogen III oxidase, DnaJ-like chaperone, ethanolamine utilization protein, F-box protein, Fe—S subunit of oxidoreductase, flavodoxin, glucose-6-phosphate 1-dehydrogenase, glutaredoxin, glutathione S-transferase, glycerol-3-phosphate dehydrogenase, heat shock transcription factor, HesB/YadR/YfhF family protein, homocitrate synthase, lipoprotein precursor, metal-dependent hydrolase, monothiol glutaredoxin, NADH-quinone oxidoreductase subunit, nitrate/nitrite transport protein, oxidoreductase, peptidyl-prolyl cis-trans isomerase, permease protein of ABC transporter, phosphate import ATP-binding protein, phosphoadenosine phosphosulfate reductase , phosphoribosylformyl glycinamidine synthase subunit, protein kinase, protein phosphatase, purine nucleoside phosphorylase, recombinase A, sll1032-protein, sodium/proton antiporter, ThiF family protein, threonine dehydratase, threonine efflux protein, transcription factor, or ymr013c-protein activity, respectively, or an antibody against polypeptides as shown in the respective line in Table II, application no. 11, columns 5 or 8, or fragments or homologs thereof which can be produced by standard techniques utilizing the polypeptid of the present invention or fragment thereof, i.e., the polypeptide of this invention (FCRP). Preferred are monoclonal antibodies.

for the disclosure of this paragraph see [0381.1.8.8] above.

In one embodiment, the present invention relates to a polypeptide as depicted in the respective line in Table II, especially in Table IIA or especially in Table IIB, application no. 11, columns 5 or 8, or fragment or homolog thereof or as coded by the nucleic acid molecule shown in the respective line in Table I, especially Table IA or especially Table IB, application no. 11, columns 5 or 8, preferably the coding region thereof, or fragment or homolog thereof.

In an embodiment, the present invention relates to a polypeptide comprising or consisting of a consensus sequence shown in the respective line in Table IV, application no. 11, column 8 or at least one polypeptide motif shown in the respective line in Table IV, application no. 11, column 8.

In an embodiment thereof, the present invention relates to a polypeptide comprising or consisting of a consensus sequence shown in the respective line in Table IV, application no. 11, column 8 or comprising at least one polypeptide motif shown in the respective line in Table IV, application no. 11, column 8, whereby not more than 20, 15, 10, 9, 8, 7, 6, 5, 4, 3, 2, or 1, or 0 of the amino acids can be replaced by any amino acid.

In another embodiment thereof, the present invention relates to a polypeptide comprising or consisting of a consensus sequence shown in the respective line in Table IV, application no. 11, column 8 or comprising at least one polypeptide motif shown in the respective line in Table IV, application no. 11, column 8, whereby not more than 15%, 10%, 5%, 4%, 3%, 2% or1% or 0% of the amino acids defined as distinct specific amino acids are/is replaced by another amino acid.

In another embodiment thereof, the present invention relates to a polypeptide comprising or consisting of a consensus sequence shown in the respective line in Table IV, application no. 11, column 8 or comprising at least one polypeptide motif shown in the respective line in Table IV, application no. 11, column 8, whereby not more than 20, 15, 10, 9, 8, 7, 6, 5, 4, 3, 2, or 1, or 0 amino acids are inserted into the consensus sequence or polypeptide motif.

In an embodiment of the present invention, the polypeptide of the present invention comprises or consists of a consensus sequence or at least one polypeptide motif shown in the respective line in Table IV, application no. 11, column 8, whereby said polypeptide distinguishes over the sequence depicted in the respective line in Table II, application no. 11, columns 5 or 8 by one or more amino acids. In one embodiment, said polypeptide of the invention distinguishes over the sequence shown in the respective line in Table II, application no. 11, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 11, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in the respective line in Table II, application no. 11, columns 5 and 8.

In an embodiment, the invention relates to a polypeptide conferring an increase in the respective fine chemical palmitic acid in a non-human organism, especially a microorganism or a plant, or a part thereof, being encoded by the nucleic acid molecule of the invention or used in the process of the invention and having a sequence which distinguishes over the sequence as shown in the respective line in Table II, application no. 11, columns 5 or 8 by one or more amino acids (but not exceeding 5%, preferably 4%, 3%, 2%, 1%, 0.5%, 0.2% or 0.1%). In an embodiment, said polypeptide of the invention does not comprise or consist of the sequence shown in the respective line in Table II, application no. 11, columns 5 or 8. In an embodiment, said polypeptide of the present invention is less than 100%, 99.999%, 99.99%, 99.9% or 99% identical. In one embodiment, said polypeptide which differs at least in one or more amino acids (but not exceeding 5%, preferably 4%, 3%, 2%, 1%, 0.5%, 0.2% or 0.1%) from the polypeptide shown in the respective line in Table II, application no. 11, columns 5 and 8 does not comprise a protein of the sequence shown in the respective line in Table II A and/or II B, application no. 11, columns 5 or 8.

In an embodiment, the present invention relates to a polypeptide having the activity of the protein as shown in the respective line in Table II, application no. 11, column 3, which distinguishes over the sequence depicted in the respective line in Table II, application no. 11, columns 5 or 8 by one or more amino acids, preferably by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 11, columns 5 and 8 by not more than 80%, 70%, 60%, 50%, 40%, 30%, 20%, 10%, 5%, 4%, 3%, 2%, 1% or 0.5%.

In another embodiment the polypeptide of the invention takes the form of a preprotein consisting of a plastidial or mitochondrial transit peptide joint to a polypeptide having the activity of the protein as shown in Table II, column 3, from which the transit peptide is preferably cleaved off upon transport of the preprotein into the organelle for example into the plastid or mitochondrion.

In another embodiment the polypeptide of the invention takes not the form of a preprotein.

[0390.1.8.11] to [0391.1.8.11] for the disclosure of these paragraphs see [0390.1.8.8] to [0391.1.8.8] above.

A polypeptide of the invention can participate in the process of the present invention. The polypeptide or a portion thereof comprises preferably an amino acid sequence, which is sufficiently homologous to an amino acid sequence shown in the respective line in Table II A and/or II B, application no. 11, columns 5 or 8.

Further, the polypeptide can have an amino acid sequence which is encoded by a nucleotide sequence which hybridizes, preferably hybridizes under stringent conditions as described above, to a nucleotide sequence of the nucleic acid molecule of the present invention. Accordingly, the polypeptide has an amino acid sequence which is encoded by a nucleotide sequence that is at least about 35%, 40%, 45%, 50%, 55%, 60%, 65%, 70%, 75%, 80%, 85%, 90%, 91%, 92%, 93%, 94%, 95%, 96%, 97%, 98%, 99%, 99.5% or more homologous to one of the amino acid sequences as shown in the respective line in Table II A and/or II B, application no. 11, columns 5 or 8, or fragments thereof. The preferred polypeptide of the present invention preferably possesses at least one of the activities according to the invention and described herein. A preferred polypeptide of the present invention includes an amino acid sequence encoded by a nucleotide sequence which hybridizes, preferably hybridizes under stringent conditions, to a nucleotide sequence shown in the respective line in Table I A and/or I B, application no. 11, columns 5 or 8, preferably the coding region thereof, or fragments thereto or which is homologous thereto, as defined above.

The polypeptide of the present invention can vary from the sequences shown in the respective line in Table II A and/or II B, application no. 11, columns 5 or 8 in amino acid sequence due to natural variation or mutagenesis, as described in detail herein. Accordingly, the polypeptide comprise an amino acid sequence which is at least about 35%, 40%, 45%, 50%, 55%, 60%, 65%, 70%, 75%, 80%, 85% or 90, 91%, 92%, 93%, 94%, 95%, 96%, 97%, 98%, 99%, 99.5% or more homologous to an entire amino acid sequence shown in the respective line in Table II A and/or II B, application no. 11, columns 5 or 8.

Biologically active portions of a polypeptide of the present invention include peptides comprising amino acid sequences derived from the amino acid sequence of the polypeptide of the present invention or used in the process of the present invention, e.g., the amino acid sequence shown in the respective line in Table II, application no. 11, columns 5 or 8 or the amino acid sequence of a protein homologous thereto, which include fewer amino acids than a full length polypeptide of the present invention or used in the process of the present invention or the full length protein which is homologous to a polypeptide of the present invention or used in the process of the present invention depicted herein, and exhibit at least one activity of a polypeptide of the present invention or used in the process of the present invention.

for the disclosure of this paragraph see [0396.1.8.8] above.

Manipulation of the nucleic acid molecule of the invention may result in the production of a protein having differences from the protein as shown in the respective line in Table II, application no. 11, column 5 or 8. Differences shall mean at least one amino acid different from the sequences as shown in the respective line in Table II, application no. 11, column 5 or 8, preferably at least 2, 3, 4, 5, 6, 7, 8, 9 or 10 amino acids (especially but not exceeding 0.1%, preferably 0.2%, 0.5%, 1%, 2%, 3% 4% or 5%), more preferably at least 15, 20, 25, 30, 35, 40, 45 or 50 amino acids (especially but not exceeding 0.5%, preferably 1%, 2%, 3% 4%, 5%, 10% or 25%) different from the sequences as shown in the respective line in Table II, application no. 11, column 5 or 8. These proteins may be improved in efficiency or activity, or may be more stable and therefore present in greater numbers in the cell as compared to the wild-type cell.

Any mutagenesis strategies for the polypeptide of the present invention or the polypeptide used in the process of the present invention to result in increasing said activity are not meant to be limiting; variations on these strategies will be readily apparent to one skilled in the art. Using such strategies, and incorporating the mechanisms disclosed herein, the nucleic acid molecule and polypeptide of the invention may be utilized to generate algae, ciliates, fungi, other microorganisms like C. glutamicum or plants or parts thereof, expressing proteins as shown in the respective line in Table II, application no. 11, column 5 or 8, or mutated proteins thereof. The nucleic acid molecules and polypeptide molecules of the invention are expressed such that the yield, production, and/or efficiency of production of a desired compound is improved.

[0399.1.8.11] to [0401.1.8.11] for the disclosure of these paragraphs see [0399.1.8.8] to [0401.1.8.8] above.

In one embodiment, a protein (=polypeptide) as shown in the respective line in Table II, application no. 11, column 5 or 8 refers to a polypeptide having an amino acid sequence corresponding to the polypeptide of the invention, especially to a polypeptide as shown in Table II, application no. 11, columns 5 or 8 or homologs thereof, or used in the process of the invention, whereas a “non- inventive protein or polypeptide” or “other polypeptide” refers to a polypeptide having an amino acid sequence corresponding to a protein which is not substantially homologous to a polypeptide of the invention, preferably which is not substantially homologous to a polypeptide or protein as shown in the respective line in Table II, application no. 11, column 5 or 8 or homologs thereof, e.g., a protein which does not confer the activity described herein and which is derived from the same or a different organism.

[0403.1.8.11] to [0409.1.8.11] for the disclosure of these paragraphs see [0403.1.8.8] to [0409.1.8.8] above.

As the nucleic acid molecules of the invention and/or the expression thereof is related to the synthesis of the respective fine chemical palmitic acid its function as a probe extends to the detection of microorganisms, plant tissues, plants, plant variets, plant ecotypes or plant genera with varying, advantageously increased, capability or potential for synthesis of the respective fine chemical palmitic acid. Therefore in one embodiment the present invention relates to a method for analyzing the capability or potential of a plant tissue, a plant, a plant variety or ecotype to produce the respective fine chemical palmitic acid by using the respective antibody of the invention as a probe to detect the amount of the polypeptide encoded by said nucleic acid molecule of the invention in a non-human organism in comparison to another organism.

Therefore in one embodiment the invention relates to a method to probe a non-human organism for its capability for the production of the respective fine chemical comprising the steps of
a) analyzing the sequence and/or the expression of the respective nucleic acid molecule of the invention in least two non-human organisms which differ in the capability to produce the respective fine chemical;
b) associate the different sequence and/or expression level of the nucleic acid molecules of the invention with their different capability to produce the respective fine chemical;
c) analyze the sequence and/or the expression of the nucleic acid molecule of the invention in at least a third non-human organism;
d) predict the capability of the at least third organism to produce the respective fine chemical based by comparing the sequence and/or expression of the nucleic acid of the invention to the sequences and/or expression levels of the nucleic acid molecule of the invention of the at least two organisms analyzed in step a.

[0411.1.8.11] to [0430.1.8.11] for the disclosure of these paragraphs see [0411.1.8.8] to [0430.1.8.8] above.

Accordingly the present invention relates to any cell transgenic for any nucleic acid characterized as part of the invention, e.g. conferring the increase of the respective fine chemical palmitic acid in a cell or a non-human organism or a part thereof, e.g. the nucleic acid molecule of the invention, the nucleic acid construct of the invention, the vector of the invention, the expression cassette according to the invention, or a nucleic acid molecule encoding the polypeptide of the invention, e.g. encoding a polypeptide having an activity as the protein as shown in the respective line in Table II, application no. 11, column 3. Due to the abovementioned activity the respective fine chemical palmitic acid content in a cell or a non-human organism is increased. For example, due to modulation or manipulation, the cellular activity is increased, in a preferred embodiment in organelles such as plastids or mitochondria, e.g. due to an increased expression or specific activity or specific targeting of the subject matters of the invention in a cell or a non-human organism or a part thereof especially in organelles such as plastids or mitochondria, or in another embodiment in the cytosol. Transgenic for a polypeptide having a protein or a protein activity means herein that due to modulation or manipulation of the genome, the activity of protein as shown in the respective line in Table II, application no. 11, column 3 or a protein as shown in the respective line in Table II, application no. 11, column 3like activity is increased in the cell or non-human organism or part thereof, especially in organelles such as plastids or mitochondria, or especially in the cytosol. Examples are described above in context with the process of the invention.

for the disclosure of this paragraph see [0432.1.8.8] above.

A naturally occurring expression cassette—for example the naturally occurring combination of the promoter of the gene encoding a protein as shown in the respective line in Table II, application no. 11, column 5 or 8, preferably the coding region thereof, or fragments or homologs thereof with the corresponding encoding gene—becomes a transgenic expression cassette when it is modified by non-natural, synthetic “artificial” methods such as, for example, mutagenesis. Such methods have been described (U.S. Pat. No. 5,565,350; WO 00/15815; also see above).

[0434.1.8.11] to [0435.1.8.11] for the disclosure of these paragraphs see [0434.1.8.8] to [0435.1.8.8] above.

Transgenic plants comprising the respective fine chemical(s) synthesized in the process according to the invention can be marketed directly without isolation of the fine chemical synthesized. In the process according to the invention, plants are understood as meaning all plant parts, plant organs such as leaf, stalk, root, tubers or seeds or propagation material or harvested material or the intact plant. In this context, the seed encompasses all parts of the seed such as the seed coats, epidermal cells, seed cells, endosperm or embryonic tissue. The fine chemical produced in the process according to the invention may, however, also be isolated from the plant. In case of palmitic acid this can be in free form or bound to proteins. Fine chemical(s) produced by this process can be harvested by harvesting the non-human organisms either from the culture in which they grow or from the field. For example, this can be done via squeezing, grinding and/or extraction, salt precipitation and/or ion-exchange chromatography of the plant parts, preferably the plant seeds, plant fruits, plant tubers and the like.

[0437.1.8.11] to [0454.1.8.11] for the disclosure of these paragraphs see [0437.1.8.8] to [0454.1.8.8] above.

In one embodiment, the present invention relates to a method for the identification of a gene product conferring the production of or an increase in the fine chemical production in a non-human cell, comprising the following steps:
(a) contacting, e.g. hybridizing, a, some or all nucleic acid molecules of a sample, e.g. cells, tissues, plants or microorganisms or a nucleic acid library, which can contain a candidate gene encoding a gene product conferring the production of or an increase in the fine chemical after expression, with the nucleic acid molecule of the present invention as shown in the respective line in column 5 or 8 of Table I A or B of application no. 11, preferably the coding region thereof, or a fragment or homolog thereof;
(b) identifying the nucleic acid molecules, which hybridize under relaxed stringent conditions with the nucleic acid molecule of the present invention in particular to the nucleic acid molecule sequence shown in the respective line in Table I, application no. 11, columns 5 or 8, preferably the coding region thereof, of a fragment or a homolog thereof, preferably in Table I B, application no. 11, columns 5 or 8, preferably the coding region thereof, of a fragment or a homolog thereof, and, optionally, isolating the full length cDNA clone or complete genomic clone;
(c) introducing the candidate nucleic acid molecules or a fragment thereof in host cells, preferably in a plant cell or a microorganism, appropriate for producing the fine chemical;
(d) expressing the identified nucleic acid molecules in the host cells for which the production of or the increased production of the fine chemical is desired;
(e) assaying the fine chemical level in the non-human host cells; and
(f) identifying the nucleic acid molecule and/or its gene product which expression confers the production of or an increase in the fine chemical level in the non-human host cell after expression compared to the wild type.

for the disclosure of this paragraph see [0456.1.8.8] above.

In another embodiment, the present invention relates to a method for the identification of a gene product conferring the production of or an increase in the fine chemical production in a cell, comprising the following steps:
(a) identifying a nucleic acid molecule of a non-human organism, which is at least 20%, 25%, 30%, 35%, 40%, 50%, 60%, 70%, 80%, 90%, 95%, 96%, 97%, 98%, 99%, 99.5% or more homolog to the nucleic acid molecule encoding a polypeptide comprising the polypeptide molecule as shown in the respective line in column 5 or 8 of Table II, application no. 11, or comprising a consensus sequence or a polypeptide motif as shown in the respective line in column 8 of Table IV, application no. 11, or being encoded by a nucleic acid molecule comprising a polynucleotide as shown in the respective line in column 5 or 8 of Table I, application no. 11, preferably the coding region thereof, or a homologue thereof as described herein, for example via homology search in a data bank;
(b) introducing the candidate nucleic acid molecule in non-human host cells, preferably in a plant cell or a microorganism, appropriate for producing the fine chemical;
(c) expressing the identified nucleic acid molecule in the non-human host cells;
(d) assaying the fine chemical level in the non-human host cells; and
(e) identifying the nucleic acid molecule and its gene product which expression confers the production of or an increase in the fine chemical level in the non-human host cell after expression compared to the wild type.

for the disclosure of this paragraph see [0458.1.8.8] above.

Further, the nucleic acid molecules disclosed herein, in particular the nucleic acid molecules shown in the respective line in column 5 or 8 of Table I A or B of application no. 11, may be sufficiently homologous to the sequences of related species such that these nucleic acid molecules may serve as markers for the construction of a genomic map in related organism or for association mapping. Furthermore natural variation in the genomic regions corresponding to nucleic acids disclosed herein, in particular the nucleic acid molecule shown in the respective line in column 5 or 8 of Table I A or B, of application no. 11 or homologous thereof, may lead to variation in the activity of the proteins disclosed herein, in particular the proteins comprising polypeptides as shown in the respective lines in column 5 or 8 of Table II A or B, of application no. 11 or their homologous, or comprising the consensus sequence or the polypeptide motif as shown in the respective line in column 8 of Table IV, and in consequence in a natural variation of an increased fine chemical production.

In consequence natural variation eventually also exists in form of more active allelic variants leading already to a relative increase in the fine chemical. Different variants of the nucleic acid molecules disclosed herein, in particular the nucleic acid molecules comprising the nucleic acid molecules as shown in the respective line in column 5 or 8 of Table I A or B of application no. 11, preferably the coding region thereof, which corresponds to different levels of increase in fine chemical, e.g. different levels of increase in fine chemical, can be indentified and used for marker assisted breeding for an enhanced production of the fine chemical.

Natural variation may also exist in the regulatory regions, e.g. the promoters of the natural genes comprising the nucleic acid molecules as shown in the respective line column 5 or 8 of Table I A or B of application no. 11, preferably the coding region thereof, which can similarly correspond to different levels of increase in the fine chemical, e.g. different levels of increase in the fine chemical can be identified and used for marker assisted breeding for an enhanced production of the fine chemical.

Accordingly, the present invention relates to a method for breeding plants for the production of the fine chemical, comprising
(a) selecting a first plant variety with an increased production of the fine chemical, e.g. based on an increased expression of a nucleic acid molecule of the invention as disclosed herein, in particular of a nucleic acid molecule comprising a nucleic acid molecule as shown in the respective line in column 5 or 8 of Table I A or B of application no. 11, preferably the coding region thereof, or fragments or homologs thereof, or a polypeptide comprising a polypeptide as shown in the respective line in column 5 or 8 of Table II A or B of application no. 11, or fragments or homologs thereof, or comprising a consensus sequence or a polypeptide motif as shown in the respective line in column 8 of Table IV, or a homolog thereof, as described herein;
(b) associating the level of increased production of the fine chemical, with the expression level or the genomic structure of a gene encoding said polypeptide or said nucleic acid molecule;
(c) crossing the first plant variety with a second plant variety, which significantly differs in its level of fine chemical; and
(d) identifying, which of the offspring varieties has got increased production of the fine chemical by the expression level of said polypeptide or nucleic acid molecule or the genomic structure of the gene(s) encoding said polypeptide or nucleic acid molecule of the invention.

[0462.1.8.11] to [0482.1.8.11] for the disclosure of these paragraphs see [0462.1.8.8] to [0482.1.8.8] above.

In a further embodiment the present invention is characterized by the features as disclosed in the following items:

### Items:

Item 1. A process for the production of the fine chemical palmitic acid
(a) increasing or generating one or more activities selected from the group consisting of 2-C-methyl-D-erythritol 4-phosphate cytidylyltransferase, 3-ketoacyl-CoA thiolase, 49747384_SOYBEAN-protein, AAS bifunctional protein, ABC metal ion transporter substrate-binding protein, acid phosphatase precursor, acid shock protein, aconitate hydratase, acyl transferase, arginine decarboxylase, aromatic acid decarboxylase, At1g19800-protein, At1g23110-protein, At2g45420-protein, ATPase epsilon subunit, b0050-protein, b1234-protein, b1248-protein, b1330-protein, b1670-protein, b1837-protein, b2032-protein, b2099-protein, b2107-protein, b2399-protein, b2682-protein, b2849-protein, b3814-protein, bifunctional protein (phosphoribosyltransferase and regulatory protein), branched chain amino acid ABC transporter ATP-binding protein, calcium-dependent protein kinase, carbamoyl-phosphate synthase subunit, carbon dioxide concentrating mechanism protein, cation-transporting ATPase, CCAAT-binding transcription factor, chlorophyllase, colanic acid biosynthesis protein, coproporphyrinogen III oxidase, DnaJ-like chaperone, ethanolamine utilization protein, F-box protein, Fe—S subunit of oxidoreductase, flavodoxin, glucose-6-phosphate 1-dehydrogenase, glutaredoxin, glutathione S-transferase, glycerol-3-phosphate dehydrogenase, heat shock transcription factor, HesB/YadR/YfhF family protein, homocitrate synthase, lipoprotein precursor, metal-dependent hydrolase, monothiol glutaredoxin, NADH-quinone oxidoreductase subunit, nitrate/nitrite transport protein, oxidoreductase, peptidyl-prolyl cis-trans isomerase, permease protein of ABC transporter, phosphate import ATP-binding protein, phosphoadenosine phosphosulfate reductase , phosphoribosylformyl glycinamidine synthase subunit, protein kinase, protein phosphatase, purine nucleoside phosphorylase, recombinase A, sll1032-protein, sodium/proton antiporter, ThiF family protein, threonine dehydratase, threonine efflux protein, transcription factor, and ymr013c-protein, in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of palmitic acid or a composition comprising palmitic acid in said non-human organism or in the culture medium surrounding said non-human organism.

Item 2. A process for the production of the fine chemical palmitic acid, which comprises
(A)
   (i) increasing or generating of the expression of; and/or
   (ii) increasing or generating the expression of an expression product of; and/or
   (iii) increasing or generating one or more activities of an expression product encoded by;
   at least one respective nucleic acid molecule comprising a nucleic acid molecule selected from the group consisting of:
   (a) a nucleic acid molecule encoding the polypeptide shown in column 5 or 8 of Table II, preferably Table II B, application no. 11, or a homolog or a fragment thereof;
   (b) a nucleic acid molecule shown in column 5 or 8 of Table I, preferably Table I B, application no.11, or a homolog or a fragment thereof (preferably the coding region thereof);
   (c) a nucleic acid molecule, which, as a result of the degeneracy of the genetic code, can be derived from a polypeptide sequence depicted in column 5 or 8 of Table II, preferably Table II B, application no.11;
   (d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 11, or the coding region thereof;
   (e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no.11;
   (i) a nucleic acid molecule encoding a polypeptide having the activity represented by a respective protein as depicted in column 5 of Table II, application no. 11;
   (j) a nucleic acid molecule which comprises a polynucleotide, which is obtained by amplifying a cDNA library or a genomic library using the primers as depicted in column 8 of Table III, application no. 11; and
   (k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
   or a nucleic acid molecule comprising a sequence which is complementary thereto; in a non-human organism or a part thereof, preferably a microorganism, a plant cell, a plant or a part thereof, as compared to a corresponding non-transformed wild type non-human organism or a part thereof; and
(B) growing the non-human organism or a part thereof under conditions which permit the production of palmitic acid or a composition comprising palmitic acid in said non-human organism or in the culture medium surrounding said non-human organism.

Item 3. A process of items 1 or 2, comprising of recovering palmitic acid in its free or bound form.

Item 4. The process of any one of items 1 to 3, comprising the following steps:
(a) selecting a non-human organism or a part thereof expressing a polypeptide encoded by the nucleic acid molecule characterized in item 2;
(b) mutagenizing the selected non-human organism or the part thereof;
(c) comparing the activity or the expression level of said polypeptide in the mutagenized non-human organism or the part thereof with the activity or the expression of said polypeptide of the selected non-human organisms or the part thereof;
(d) selecting the mutated non-human organisms or parts thereof, which comprise an increased activity or expression level of said polypeptide compared to the selected non-human organism or the part thereof;
(e) optionally, growing and cultivating the non-human organisms or the parts thereof;
and
(f) recovering, and optionally isolating, the free or bound palmitic acid produced by the selected mutated non-human organisms or parts thereof.

Item 5. The process of any one of items 1 to 4 wherein the activity of said protein or the expression of said nucleic acid molecule is increased or generated transiently or stably.

Item 6. An isolated nucleic acid molecule comprising a nucleic acid molecule selected from the group consisting of:
(a) a nucleic acid molecule encoding a polypeptide shown in Table II, application no. 11, column 5 or 8, preferably shown in Table II B, application no. 11, column 8, or a homolog or a fragment thereof;
(b) a nucleic acid molecule shown in Table I, application no. 11, column 5 or 8, preferably shown in Table I B, application no. 11, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 11, preferably in column 8 of Table II B, application no. application no.,
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 3, preferably shown in column 8 of Table I B, application no. 11, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 11;
(i) a nucleic acid molecule encoding a polypeptide having the activity represented by a respective protein as depicted in column 5 of Table II, application no. 11;
(j) a nucleic acid molecule comprising a polynucleotide which is obtained by amplifying a cDNA library or a genomic library using the primers in Table III, application no. 11, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
or a nucleic acid molecule comprising a sequence which is complementary thereto.

Item 7. A nucleic acid construct which confers the expression of the nucleic acid molecule as defined in item 6, comprising one or more regulatory elements.

Item 8. An expression cassette comprising
(a) a promoter, preferably selected from the group consisting of Big35S, PCUbi, Super and USP;
(b) a nucleic acid molecule as defined in item 6.

Item 9. A vector comprising the nucleic acid molecule as defined in item 6 or the nucleic acid construct as claimed in item 7 or the expression cassette as claimed in item 8.

Item 10. A host cell or a plant organelle, which has been transformed with the vector as claimed in item 9 or the nucleic acid molecule as defined in item 6 or the nucleic acid construct as claimed in item 7 or the expression cassette as claimed in item 8.

Item 11. The host cell of item 10, which is a plant cell or a microorganism.

Item 12. A process for producing a polypeptide, wherein the polypeptide is expressed in a host cell as claimed in item 10 or 11.

Item 13. A polypeptide produced by the process as claimed in item 12 or encoded by the nucleic acid molecule as defined in item 6.

Item 14. An antibody, which binds specifically to the polypeptide as claimed in item 13.

Item 15. A plant tissue, propagation material, harvested material or a plant or a part thereof comprising the host cell as claimed in item 10 or 11.

Item 16. A process for the identification of a compound conferring an increase in palmitic acid production in a non-human organism, comprising the steps:
(a) culturing a plant cell or tissue or microorganism or maintaining a plant expressing the polypeptide encoded by the nucleic acid molecule as defined in item 6 conferring an increase in the amount of palmitic acid in a non-human organism or a part thereof and a readout system capable of interacting with the polypeptide under suitable conditions which permit the interaction of the polypeptide with said readout system in the presence of a compound or a sample comprising a plurality of compounds and capable of providing a detectable signal in response to the binding of a compound to said polypeptide under conditions which permit the expression of said readout system and of the polypeptide encoded by the nucleic acid molecule of item 6 conferring an increase in the amount of palmitic acidin a non-human organism or a part thereof;
(b) identifying if the compound is an effective agonist by detecting the presence or absence or increase of a signal produced by said readout system.

Item 17. A method for the production of an agricultural composition comprising the steps of the method of item 16 and formulating the compound identified in item 16 in a form acceptable for an application in agriculture.

Item 18. A composition comprising the nucleic acid molecule as defined in item 6, the polypeptide as claimed in item 13, the nucleic acid construct as claimed in item 7, the expression cassette as claimed in item 8, the vector as claimed in item 9, the compound as claimed in item 16, the antibody as claimed in item 14, and optionally an agricultural acceptable carrier.

Item 19. Use of the nucleic acid molecule as defined in item 6 for the identification of a nucleic acid molecule conferring an increase in palmitic acid after expression.

Item 20. Cosmetic, pharmaceutical, food or feed composition comprising the nucleic acid molecule as defined in item 6, the polypeptide as claimed in item 13, the nucleic acid construct as claimed in item 7, the expression cassette as claimed in item 8, the vector as claimed in item 9, the antibody as claimed in item 14, the plant or a part thereof, or plant tissue as claimed in item 15, the harvested material or propagation material as claimed in item 15 or the host cell as claimed in items 10 or 11.

Item 21. Use of the nuclei acid molecule as defined in item 6, the polypeptide as claimed in item 13, the nucleic acid construct as claimed in item 7, the expression cassette as claimed in item 8, the vector as claimed in claim 9, the plant or plant tissue as claimed in item 15, or the host cell as claimed in item 10 to 11 for the production of plant resistant to a herbicide inhibiting the production of palmitic acid.

[0483.1.8.11] to [0494.1.8.11] for the disclosure of this paragraph see [0483.1.8.8] to [0494.1.8.8] above.

### Example 11a

### PCR Amplification of the Sequences

Unless otherwise specified, standard methods as described in Sambrook et al., Molecular Cloning: A laboratory manual, Cold Spring Harbor 1989, Cold Spring Harbor Laboratory Press are used.

The inventive sequences as shown in the respective line in Table I, column 5, preferably the coding region thereof, were amplified by PCR as described in the protocol of the Pfu Ultra, Pfu Turbo or Herculase DNA polymerase (Stratagene). The composition for the protocol of the Pfu Ultra, Pfu Turbo or Herculase DNA polymerase was as follows: 1× PCR buffer (Stratagene), 0.2 mM of each dNTP, 100 ng genomic DNA of Saccharomyces cerevisiae (strain S288C; Research Genetics, Inc., now Invitrogen), Escherichia coli (strain MG1655; E. coli Genetic Stock Center), Synechocystis sp. (strain PCC6803), Azotobacter vinelandii (strain N.R. Smith,16), Thermus thermophilus (HB8) or 50 ng cDNA from various tissues and development stages of Arabidopsis thaliana (ecotype Columbia), Physcomitrella patens, Glycine max (variety Resnick), Brassica napus, Oryza sativa or Zea mays (variety B73, Mo17, A188), 50 pmol forward primer, 50 pmol reverse primer, with or without 1 M Betaine, 2.5 u Pfu Ultra, Pfu Turbo or Herculase DNA polymerase.

The amplification cycles were as follows:

1 cycle of 2-3 minutes at 94-95° C., then 25-36 cycles with 30-60 seconds at 94-95° C., 30-45 seconds at 50-60° C. and 210-480 seconds at 72° C., followed by 1 cycle of 5-10 minutes at 72° C., then 4-16° C.—preferably for Saccharomyces cerevisiae, Escherichia coli, Synechocystis sp., Azotobacter vinelandii, Thermus thermophilus.

In case of Arabidopsis thaliana, Brassica napus, Glycine max, Oryza sativa, Physcomitrella patens, Zea mays the amplification cycles were as follows:

1 cycle with 30 seconds at 94° C., 30 seconds at 61° C., 15 minutes at 72° C., then 2 cycles with 30 seconds at 94° C., 30 seconds at 60° C., 15 minutes at 72° C., then 3 cycles with 30 seconds at 94° C., 30 seconds at 59° C., 15 minutes at 72° C., then 4 cycles with 30 seconds at 94° C., 30 seconds at 58° C., 15 minutes at 72° C., then 25 cycles with 30 seconds at 94° C., 30 seconds at 57° C., 15 minutes at 72° C., then 1 cycle with 10 minutes at 72° C., then finally 4-16° C. RNAs were generated with the RNeasy Plant Kit according to the standard protocol (Qiagen) and Superscript II Reverse Transkriptase was used to produce double stranded cDNA according to the standard protocol (Invitrogen).

ORF specific primer pairs for the genes to be expressed are shown in the respective line in Table III, column 8. The following adapter sequences were added to Saccharomyces cerevisiae ORF specific primers (see Table III) for cloning purposes:

SEQ ID NO: 20

| | | |
|---|---|---|
| i) | foward primer: | 5′-GGAATTCCAGCTGACCACC-3′ |

SEQ ID NO: 21

| | | |
|---|---|---|
| ii) | reverse primer: | 5′-GATCCCCGGGAATTGCCATG-3′ |

These adaptor sequences allow cloning of the ORF into the various vectors containing the Resgen adaptors, see table column E of Table c.

The following adapter sequences were added to Saccharomyces cerevisiae, Escherichia coli, Synechocystis sp., Azotobacter vinelandii, Thermus thermophilus, Arabidopsis thaliana, Brassica napus, Glycine max, Oryza sativa, Physcomitrella patens, or Zea mays ORF specific primers for cloning purposes:

| SEQ ID NO: 22 | | |
|---|---|---|
| iii) | forward primer: | 5′-TTGCTCTTCC-3′ |
| SEQ ID NO: 23 | | |
| iiii) | reverse primer: | 5′-TTGCTCTTCG-3′ |

The adaptor sequences allow cloning of the ORF into the various vectors containing the Colic adaptors, see table column E of Table c.

Therefore for amplification and cloning of Saccharomyces cerevisiae SEQ ID NO: 66772, a primer consisting of the adaptor sequence i) and the ORF specific sequence SEQ ID NO: 67180 and a second primer consisting of the adaptor sequence ii) and the ORF specific sequence SEQ ID NO: 67181 were used.

For amplification and cloning of Escherichia coli SEQ ID NO: 8920, a primer consisting of the adaptor sequence iii) and the ORF specific sequence SEQ ID NO: 8930 and a second primer consisting of the adaptor sequence iiii) and the ORF specific sequence SEQ ID NO: 8931 were used.

For amplification and cloning of Synechocystis sp. SEQ ID NO: 11423, a primer consisting of the adaptor sequence iii) and the ORF specific sequence SEQ ID NO: 11465 and a second primer consisting of the adaptor sequence iiii) and the ORF specific sequence SEQ ID NO: 11466 were used.

For amplification and cloning of Arabidopsis thaliana SEQ ID NO: 1061, a primer consisting of the adaptor sequence iii) and the ORF specific sequence SEQ ID NO: 1293 and a second primer consisting of the adaptor sequence iiii) and the ORF specific sequence SEQ ID NO: 1294 were used.

For amplification and cloning of Azotobacter vinelandii SEQ ID NO: 6674, a primer consisting of the adaptor sequence iii) and the ORF specific sequence SEQ ID NO: 6806 and a second primer consisting of the adaptor sequence iiii) and the ORF specific sequence SEQ ID NO: 6807 were used.

For amplification and cloning of Glycine max SEQ ID NO: 69, a primer consisting of the adaptor sequence iii) and the ORF specific sequence SEQ ID NO: 343 and a second primer consisting of the adaptor sequence iiii) and the ORF specific sequence SEQ ID NO: 344 were used.

For amplification and cloning of Thermus thermophilus SEQ ID NO: 12974, a primer consisting of the adaptor sequence iii) and the ORF specific sequence SEQ ID NO: 13372 and a second primer consisting of the adaptor sequence iiii) and the ORF specific sequence SEQ ID NO: 13373 were used.

Following these examples every sequence disclosed in Table I, preferably column 5, especially the coding region thereof can be cloned by fusing the adaptor sequences to the respective specific primers sequences as disclosed in Table III, column 8 using the respective vectors shown in Table c.

[0496.1.8.11] to [0499.1.8.11] for the disclosure of these paragraphs see [0496.1.8.8] to [0499.1.8.8] above.

### Example 11e

### Cloning of Inventive Sequences as Shown in Table I, Column 5 in the Different Expression Vectors

For cloning of for example SEQ ID NO: 8920 from Escherichia coli or any other ORF from Escherichia coli, Synechocystis sp., Azotobacter vinelandii, Thermus thermophilus, Arabidopsis thaliana, or Glycine max the vector DNA was treated with the restriction enzymes Pacl and Ncol following the standard protocol (MBI Fermentas).

For cloning for example the ORFs of SEQ ID NO: 66772 from Saccharomyces cerevisiae or any other ORF from Saccharomyces cerevisiae into vectors containing the Resgen adaptor sequence the respective vector DNA was treated with the restriction enzyme Ncol.

For cloning of for example ORFs SEQ ID NO: 119408 from Saccharomyces cerevisiae or any other ORF from Saccharomyces cerevisiae into vectors containing the Colic adaptor sequence, the respective vector DNA was treated with the restriction enzymes Pacl and Ncol following the standard protocol (MBI Fermentas).

In all cases the reaction was stopped by inactivation at 70° C. for 20 minutes and purified over QlAquick or NucleoSpin Extract II columns following the standard protocol (Qiagen or Macherey-Nagel).

Then the PCR-product representing the amplified ORF with the respective adapter sequences and the vector DNA were treated with T4 DNA polymerase according to the standard protocol (MBI Fermentas) to produce single stranded overhangs with the parameters 1 unit T4 DNA polymerase at 37° C. for 2-10 minutes for the vector and 1-2 u T4 DNA polymerase at 15-17° C. for 10-60 minutes for the PCR product representing SEQ ID NO: 8920.

The reaction was stopped by addition of high-salt buffer and purified over QlAquick or NucleoSpin Extract II columns following the standard protocol (Qiagen or Macherey-Nagel).

According to this example the skilled person is able to clone all sequences disclosed in Table I, preferably column 5 or column 8, especially the coding region thereof.

Approximately 30-60 ng of prepared vector and a defined amount of prepared amplificate were mixed and hybridized at 65° C. for 15 minutes followed by 37° C. 0.1 ° C/1 seconds, followed by 37° C. 10 minutes, followed by 0.1 ° C/1 seconds, then 4-10 ° C.

The ligated constructs were transformed in the same reaction vessel by addition of competent E. coli cells (strain DHSalpha) and incubation for 20 minutes at 1 ° C. followed by a heat shock for 90 seconds at 42° C. and cooling to 1-4° C. Then, complete medium (SOC) was added and the mixture was incubated for 45 minutes at 37° C. The entire mixture was subsequently plated onto an agar plate with 0.05 mg/ml kanamycin and incubated overnight at 37° C.

The outcome of the cloning step was verified by amplification with the aid of primers which bind upstream and downstream of the integration site, thus allowing the amplification of the insertion. The amplifications were carried out as described in the protocol of Taq DNA polymerase (Gibco-BRL).

The amplification cycles were as follows:

1 cycle of 1-5 minutes at 94° C., followed by 35 cycles of in each case 15-60 seconds at 94° C., 15-60 seconds at 50-66° C. and 5-15 minutes at 72° C., followed by 1 cycle of 10 minutes at 72° C., then 4-16° C.

Several colonies were checked, but only one colony for which a PCR product of the expected size was detected was used in the following steps. A portion of this positive colony was transferred into a reaction vessel filled with complete medium (LB) supplemented with kanamycin and incubated overnight at 37° C.

The plasmid preparation was carried out as specified in the Qiaprep or NucleoSpin Multi-96 Plus standard protocol (Qiagen or Macherey-Nagel).

[0501.1.8.11] to [0503.1.8.11] for the disclosure of these paragraphs see [0501.1.8.8] to [0503.1.8.8] above.

Table d showing results of plant analyses

TABLE d

| SeqID | Target | Locus | Metabolite | Source | Promotor | Method | Min | Max |
|---|---|---|---|---|---|---|---|---|
| 17451 | non- | At1g07430 | palmitic | ARA_LEAF | p-PcUBI | GC | 16 | 36 |
| | targeted | | acid | | | | | |
| 113651 | non- | At1g19670 | palmitic | ARA_LEAF | p-PcUBI | GC | 15 | 27 |
| | targeted | | acid | | | | | |
| 17637 | non- | At1g19800 | palmitic | ARA_LEAF | p-PcUBI | GC | 18 | 29 |
| | targeted | | acid | | | | | |
| 102086 | non- | At1g23110 | palmitic | ARA_LEAF | p-PcUBI | GC | 24 | 58 |
| | targeted | | acid | | | | | |
| 1061 | non- | At1g68320 | palmitic | ARA_LEAF | p-PcUBI | GC | 16 | 84 |
| | targeted | | acid | | | | | |
| 19419 | non- | At1g72770 | palmitic | ARA_LEAF | p-PcUBI | GC | 16 | 54 |
| | targeted | | acid | | | | | |
| 1623 | non- | At2g45420 | palmitic | ARA_LEAF | p-PcUBI | GC | 18 | 48 |
| | targeted | | acid | | | | | |
| 68657 | non- | At3g02990 | palmitic | ARA_LEAF | p-PcUBI | GC | 15 | 33 |
| | targeted | | acid | | | | | |
| 22921 | non- | At3g20910 | palmitic | ARA_LEAF | p-PcUBI | GC | 19 | 41 |
| | targeted | | acid | | | | | |
| 2935 | non- | At3g62950 | palmitic | ARA_LEAF | p-PcUBI | GC | 19 | 40 |
| | targeted | | acid | | | | | |
| 3279 | non- | At4g15670 | palmitic | ARA_LEAF | p-PcUBI | GC | 16 | 75 |
| | targeted | | acid | | | | | |
| 24232 | non- | At4g18880 | palmitic | ARA_LEAF | p-PcUBI | GC | 19 | 23 |
| | targeted | | acid | | | | | |
| 68849 | non- | At4g26080 | palmitic | ARA_LEAF | p-PcUBI | GC | 16 | 18 |
| | targeted | | acid | | | | | |
| 4102 | non- | At4g33040 | palmitic | ARA_LEAF | p-PcUBI | GC | 19 | 36 |
| | targeted | | acid | | | | | |
| 4348 | non- | At4g35310 | palmitic | ARA_LEAF | p-PcUBI | GC | 16 | 40 |
| | targeted | | acid | | | | | |
| 5318 | non- | At5g57050 | palmitic | ARA_LEAF | p-PcUBI | GC | 17 | 43 |
| | targeted | | acid | | | | | |
| 25498 | non- | At5g59220 | palmitic | ARA_LEAF | p-PcUBI | GC | 19 | 400 |
| | targeted | | acid | | | | | |
| 114083 | non- | AvinDRAFT_1788 | palmitic | ARA_LEAF | p-PcUBI | GC | 17 | 33 |
| | targeted | | acid | | | | | |
| 111155 | non- | AvinDRAFT_3577 | palmitic | ARA_LEAF | p-PcUBI | GC | 18 | 49 |
| | targeted | | acid | | | | | |
| 114231 | non- | AvinDRAFT_3629 | palmitic | ARA_LEAF | p-PcUBI | GC | 16 | 51 |
| | targeted | | acid | | | | | |
| 73719 | non- | AvinDRAFT_4606 | palmitic | ARA_LEAF | p-PcUBI | GC | 18 | 57 |
| | targeted | | acid | | | | | |
| 32648 | non- | AvinDRAFT_5467 | palmitic | ARA_LEAF | p-PcUBI | GC | 17 | 61 |
| | targeted | | acid | | | | | |
| 6674 | non- | AvinDRAFT_6075 | palmitic | ARA_LEAF | p-PcUBI | GC | 18 | 31 |
| | targeted | | acid | | | | | |
| 34044 | non- | AvinDRAFT_6700 | palmitic | ARA_LEAF | p-PcUBI | GC | 21 | 29 |
| | targeted | | acid | | | | | |
| 103958 | non- | B0050 | palmitic | ARA_LEAF | p-Super | GC | 16 | 27 |
| | targeted | | acid | | | | | |
| 37400 | non- | B1186 | palmitic | ARA_LEAF | p-Super | GC | 20 | 47 |
| | targeted | | acid | | | | | |
| 112111 | non- | B1234 | palmitic | ARA_LEAF | p-Super | GC | 16 | 22 |
| | targeted | | acid | | | | | |
| 119135 | non- | B1248 | palmitic | ARA_LEAF | p-Super | GC | 15 | 22 |
| | targeted | | acid | | | | | |
| 38226 | non- | B1330 | palmitic | ARA_LEAF | p-Super | GC | 25 | 36 |
| | targeted | | acid | | | | | |
| 38266 | plastidic | B1431 | palmitic | ARA_LEAF | p-Super | GC | 16 | 25 |
| | | | acid | | | | | |
| 78154 | non- | B1589 | palmitic | ARA_LEAF | p-Super | GC | 16 | 25 |
| | targeted | | acid | | | | | |
| 38300 | non- | B1597 | palmitic | ARA_LEAF | p-Super | GC | 20 | 44 |
| | targeted | | acid | | | | | |
| 78753 | non- | B1670 | palmitic | ARA_LEAF | p-Super | GC | 16 | 37 |
| | targeted | | acid | | | | | |
| 78953 | non- | B1837 | palmitic | ARA_LEAF | p-Super | GC | 16 | 39 |
| | targeted | | acid | | | | | |
| 8920 | non- | B2032 | palmitic | ARA_LEAF | p-Super | GC | 22 | 83 |
| | targeted | | acid | | | | | |
| 112193 | non- | B2043 | palmitic | ARA_LEAF | p-Super | GC | 16 | 54 |
| | targeted | | acid | | | | | |
| 115595 | non- | B2099 | palmitic | ARA_LEAF | p-Super | GC | 18 | 27 |
| | targeted | | acid | | | | | |
| 39002 | non- | B2107 | palmitic | ARA_LEAF | p-Super | GC | 15 | 37 |
| | targeted | | acid | | | | | |
| 39237 | non- | B2399 | palmitic | ARA_LEAF | p-Super | GC | 19 | 45 |
| | targeted | | acid | | | | | |
| 115628 | non- | B2439 | palmitic | ARA_LEAF | p-Super | GC | 16 | 34 |
| | targeted | | acid | | | | | |
| 115655 | non- | B2682 | palmitic | ARA_LEAF | p-Super | GC | 22 | 28 |
| | targeted | | acid | | | | | |
| 81075 | non- | B2747 | palmitic | ARA_LEAF | p-Super | GC | 23 | 38 |
| | targeted | | acid | | | | | |
| 115719 | non- | B2790 | palmitic | ARA_LEAF | p-Super | GC | 17 | 34 |
| | targeted | | acid | | | | | |
| 115791 | plastidic | B2836 | palmitic | ARA_LEAF | p-Super | GC | 17 | 32 |
| | | | acid | | | | | |
| 81980 | non- | B2849 | palmitic | ARA_LEAF | p-Super | GC | 20 | 56 |
| | targeted | | acid | | | | | |
| 44196 | non- | B3814 | palmitic | ARA_LEAF | p-Super | GC | 19 | 41 |
| | targeted | | acid | | | | | |
| 10726 | non- | B3823 | palmitic | ARA_LEAF | p-Super | GC | 21 | 35 |
| | targeted | | acid | | | | | |
| 100675 | plastidic | B3845 | palmitic | ARA_SEED_2 | p-USP | GC | 15 | 31 |
| | | | acid | | | | | |
| 10811 | non- | GM02LC12622 | palmitic | ARA_LEAF | p-PcUBI | GC | 19 | 45 |
| | targeted | | acid | | | | | |
| 46751 | non- | GM02LC19289 | palmitic | ARA_LEAF | p-PcUBI | GC | 17 | 80 |
| | targeted | | acid | | | | | |
| 106920 | mitochondrial | Sll0368 | palmitic | ARA_LEAF | p-PcUBI | GC | 20 | 28 |
| | | | acid | | | | | |
| 94741 | plastidic | Sll0374 | palmitic | ARA_LEAF | p-PcUBI | GC | 20 | 23 |
| | | | acid | | | | | |
| 52634 | mitochondrial | Sll1031 | palmitic | ARA_LEAF | p-PcUBI | GC | 18 | 38 |
| | | | acid | | | | | |
| 85723 | mitochondrial | Sll1032 | palmitic | ARA_LEAF | p-PcUBI | GC | 16 | 27 |
| | | | acid | | | | | |
| 52660 | plastidic | Sll1056 | palmitic | ARA_LEAF | p-PcUBI | GC | 21 | 38 |
| | | | acid | | | | | |
| 54337 | mitochondrial | Sll1450 | palmitic | ARA_LEAF | p-PcUBI | GC | 19 | 29 |
| | | | acid | | | | | |
| 95047 | non- | Sll1498 | palmitic | ARA_LEAF | p-PcUBI | GC | 18 | 24 |
| | targeted | | acid | | | | | |
| 11423 | mitochondrial | Sll1545 | palmitic | ARA_LEAF | p-PcUBI | GC | 15 | 27 |
| | | | acid | | | | | |
| 85743 | mitochondrial | Sll1598 | palmitic | ARA_LEAF | p-PcUBI | GC | 17 | 28 |
| | | | acid | | | | | |
| 86447 | plastidic | Sll1848 | palmitic | ARA_LEAF | p-PcUBI | GC | 18 | 115 |
| | | | acid | | | | | |
| 11471 | mitochondrial | Sll1917 | palmitic | ARA_LEAF | p-PcUBI | GC | 16 | 23 |
| | | | acid | | | | | |
| 55385 | mitochondrial | Sll1920 | palmitic | ARA_LEAF | p-PcUBI | GC | 18 | 34 |
| | | | acid | | | | | |
| 119222 | mitochondrial | Slr0949 | palmitic | ARA_LEAF | p-PcUBI | GC | 18 | 25 |
| | | | acid | | | | | |
| 116341 | mitochondrial | Slr1099 | palmitic | ARA_LEAF | p-PcUBI | GC | 23 | 39 |
| | | | acid | | | | | |
| 116460 | mitochondrial | Slr1250 | palmitic | ARA_LEAF | p-PcUBI | GC | 19 | 33 |
| | | | acid | | | | | |
| 58324 | non- | Slr1312 | palmitic | ARA_LEAF | p-PcUBI | GC | 21 | 34 |
| | targeted | | acid | | | | | |
| 12140 | mitochondrial | Slr1791 | palmitic | ARA_LEAF | p-PcUBI | GC | 16 | 17 |
| | | | acid | | | | | |
| 12341 | mitochondrial | Slr2072 | palmitic | ARA_LEAF | p-PcUBI | GC | 19 | 42 |
| | | | acid | | | | | |
| 61553 | non- | TTC0917 | palmitic | ARA_LEAF | p-PcUBI | GC | 16 | 112 |
| | targeted | | acid | | | | | |
| 12974 | non- | TTC1550 | palmitic | ARA_LEAF | p-PcUBI | GC | 19 | 35 |
| | targeted | | acid | | | | | |
| 119347 | plastidic | Ybr092c | palmitic | ARA_LEAF | p-Super | GC | 16 | 31 |
| | | | acid | | | | | |
| 119408 | non- | Ydl022w | palmitic | ARA_SEED_2 | p-PcUBI | GC | 13 | 39 |
| | targeted | | acid | | | | | |
| 122518 | non- | Ydl101c | palmitic | ARA_LEAF | Big35S | GC | 20 | 36 |
| | targeted | | acid | | | | | |
| 14706 | plastidic | Ydr131c | palmitic | ARA_LEAF | p-Super | GC | 19 | 37 |
| | | | acid | | | | | |
| 64964 | plastidic | Yjl073w | palmitic | ARA_LEAF | p-Super | GC | 18 | 39 |
| | | | acid | | | | | |
| 66772 | non- | Ylr304c | palmitic | ARA_LEAF | Big35S | GC | 16 | 38 |
| | targeted | | acid | | | | | |
| 91958 | plastidic | Ymr013c | palmitic | ARA_LEAF | p-Super | GC | 16 | 26 |
| | | | acid | | | | | |
| 118212 | plastidic | Ypl271w | palmitic | ARA_LEAF | p-Super | GC | 19 | 33 |
| | | | acid | | | | | |
| 69 | non- | 49747384_SOYBEAN | palmitic | ARA_LEAF | p-PcUBI | GC | 15 | 29 |
| | targeted | | acid | | | | | |
| 16883 | plastidic | 59582753_SOYBEAN | palmitic | ARA_LEAF | p-PcUBI | GC | 16 | 42 |
| | | | acid | | | | | |

Column 1 shows the SEQ ID NO, Column 2 shows the expression type (targeted or non-targeted), Column 3 shows the “gene name” (locus), Column 4 shows the metabolite analyzed, Column 5 indicates the A. thaliana source tissue analyzed, Column 6 indicates the used promoter for expression, Column 7 indicates the analytical method. Columns 8 and 9 show the minimum and the maximum increase of the analyzed metabolite (in percent) in comparison to the wild type (=ratio_by_weight, given as percent increase).

When the analyses were repeated independently, all results proved to be significant.

[0505.1.8.11] to [0515.1.8.11] for the disclosure of these paragraphs see [0505.1.8.8] to [0515.1.8.8] above.

In a further embodiment, the present invention relates in paragraphs [0000.1.8.12] to [0514.1.8.12] to a further process for the production of the fine chemical selected from the group comprising 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid and oleic acid, as defined below and corresponding embodiments as described herein as follows.

for the disclosure of this paragraph see [0001.1.8.8] above.

Oils and fats, which chemically are glycerol esters of fatty acids (triacylglycerols (TAGs)), play a major role in nutrition but more and more in nonfood applications such as lubricants, hydraulic oil, biofuel, or oleochemicals for coatings, plasticizer, soaps, and detergents (W. Lohs and W. Friedt, in “Designer Oil Crops”, Ed. D. J. Murphy, (VCH, Weinheirn, Germany, 1993)). The ideal oil for industrial application would consist of a particular type of fatty acid that could be supplied constantly at a competitively low price as compared with raw materials based on mineral oil products. Furthermore, such a fatty acid may have a reactive group in addition to the carboxyl function to provide an additional target for chemical modifications (Töpfer et al., Science 268, 681, (1995)).

The various fatty acids and triglycerides are mainly obtained from microorganisms such as fungi, from animals such as fish or from oil-producing plants including phytoplankton and algae, such as soybean, oilseed rape, sunflower and others, where they are usually obtained in the form of their triacylglycerides.

Further sources of fatty acids are membrane lipids of organisms. Preferably lipids are phopholipids and/or glycolipids, more preferably glycerophospholipids, galactolipids and/or sphingolipids.

The saturated fatty acid with 12 carbon atoms lauric acid is the main fatty acid in coconut oil and in palm kernel oil and is believed to have antimiicrobial properties, It is also found in human, cow's and goat's milk.

Myristic acid is a saturated fatty acid with forteen carbon atoms and is found in form of its triglyceride in Mysterica fragans, but also in palm kernel oil, cocunut oil and it is a minor component in may animal fats. It is also found in the spermacetin of the sperm whale. The isopropylester of myristic acid is used in cosmetics and topical medical preparations where good absorption through the skin is desired.

Heptadecanoic (17:0) is occuring in nature only in minor amounts. This fatty acid with an odd number of carbon atoms is present in trace amounts in plants, in triglycerides from brazil-nut oil, Dracocephalum moldavica oil, poppy-seed, palm, almond, sunflower or soybean. It can be isolated from tallow (1%), specially from subcutaneous adipose tissue in subcutaneous fat from lambs.

Heptadecanoic acid can be ingredient of satiety agents or fungicide composition. It is further used as ingredient in cosmetics, pharmaceuticals and in feed and food, like baking adjuvants as disclosed in US 2003/143312 or according to US 2004/097392 as component in surfactant systems.

Heptadecanoic acid is mainly used as an internal standard in quantification of fatty acids. It can be further useful in treatment of neurological diseases which may be caused by yeast, fungi or prions based on yeast or fungal etiology (U.S. Pat. No. 6,652,866) or in antikeratolytic-wound healing compositions (U.S. Pat. No. 5,641,814).Heptadecanoic acid was produced up to now in higher amount primarily by organic synthesis.

Very long chain fatty acids (VLCFAs) such as eicosanoic acid (20:0) and behenic acid (C22:0) are produced by plants. Hereby a membrane-bound fatty acid elongation complex (elongase, FAE) using acyl-CoA substrates is used. The first reaction of elongation involves condensation of malonyl-CoA with a long chain substrate producing a β3-ketoacyl-CoA. Subsequent reactions are reduction of β3-hydroxyacyl-CoA, dehydration to an enoyl-CoA, followed by a second reduction to form the elongated acyl-CoA. The β3-ketoacyl-CoA synthase (KCS) catalyzing the condensation reaction plays a key role in determining the chain length of fatty acid products found in seed oils and is the rate-limiting enzyme for seed VLCFA production (Lassner et al., Plant Cell, 8, 281 (1996)).

Although such very long chain fatty acids are minor components of the lipid membranes of the body, they undoubtedly perform valuable functions, apparently helping to stabilize membranes, especially those in peripheral nerve cells.

Eicosanoic acid occurs in appreciable quantities in peanut oil and Behenic acid is a component of rapeseed oil (up to 2%) as well as peanut oil (1-5%). It seems that these two acids are beneath other very-long-chain fatty acid sessential components for the vitality of heigher plants.

Eicosanoid acid is used as a lubricant and as an emulsifier in industrial applications. Behenic acid is used to give hair conditioners and moisturizers their smoothin properties.

Due to the interesting physiological roles and the nutritional, cosmetic and agrotechnical potential of these acids there is a need to identify genes encoding enzymes and proteins being involved in th ebiosynthesis of such acids.

2-Hydroxy fatty acids are synthesised in animal and plant tissues, and are often major constituents of the sphingolipids. Sphingolipids with 2-hydroxy fatty acid are found in most organisms including plants, yeast, worms, vertebrate animals, and some bacterial species.

In plants more than 95% of the fatty acid component of the ceramides and sphingolipids is alpha-hydroxylated. The acyl groups of ceramides tend to consist of long-chain (C16 up to C26 but occasionally longer) odd- and even-numbered saturated or monoenoic fatty acids and related 2-D-hydroxy fatty acids, both in plant and animal tissues.

Typical plant sphingolipids are made up by the long-chain sphingosine backbone which is glycosylated and amide-linked to an usually hydroxylated very-long-chain fatty acid, called cerebroside. Cerebrosides are essential constituents of the plasma membrane involved in various physiological functions including signaling, exocytosis, anchoring of proteins, and vesicular protein transport (Matthes et al., Z. Naturforsch. 57C, 843 (2002)).

In mammals, 2-hydroxysphingolipids are present abundantly in brain because the major myelin lipids galactosylceramides and sulfatides contain 2-hydroxy fatty acids. In mammals, 2-hydroxy fatty acid-containing sphingolipids are uniquely abundant in nervous and epidermal tissues. In mammalian central and peripheral nervous systems, galactosylceramides and sulfatides (3-sulfate ester of galactosylceramide) are major lipid components of myelin. These glycosphingolipids contain a high proportion (about 50%) of 2-hydroxy fatty acid and are critical components of myelin.

In the yeast Saccharomyces cerevisiae most sphingolipids contain 2-hydroxy fatty acid. COS7 cells expressing human FA2H contained 3-20-fold higher levels of 2-hydroxyceramides (C16, C18, C24, and C24:1) and 2-hydroxy fatty acids compared with control cells (Alderson et al., J. Biol. Chem. 279 (47) 48562 (2004).

The 2-hydroxylation occurs during de novo ceramide synthesis and is catalyzed by fatty acid 2-hydroxylase (also known as fatty acid alpha-hydroxylase). No free hydroxy fatty acid or hydroxy fatty acid CoA has ever been reported; the hydroxylated product always appeared as a component of ceramide or cerebroside (Hoshi et al., J. Biol. Chem. 248, 4123 (1973). The alphahydroxylation involves the direct hydroxylation of a sphingolipid-bound fatty acid. (Kayal et al., J. Biol. Chem. 259 (6), 3548 (1984)).

Hydroxylated fatty acids initiate inflammation in the soft tissues and regulate the immune response.

The 2-hydroxyl group in sphingolipids has a profound effect in the lipid organization within membranes because of its hydrogenbonding capability.

Alpha-hydroxy-palmitic acid (OH 16:0) is mainly a building block of plant sphingolipids, for example soy glucosylceramide (GIcCer), which consists predominantly of a 4,8-sphingadiene backbone and alpha-hydroxy-palmitic acid. Soy GIcCer suppress tumorigenesis and gene expression in mice (Symolon et al., J. Nutr. 134(5), 1157 (2004)).

Another hydroxylated fatty acid being a building block of cerebrosides is the 2-hydroxy-nervonic acid (OH 24:1). 2-hydroxy-15-tetracosenoic acid (hydroxynervonic acid) is constituent of the ceramide part of cerebrosides (glycosphingolipides found mainly in nervous tissue and in little amount in plants). The occurrence of 2-hydroxy nervonic acid is characteristic for the leaf cerebrosides of some chilling-resistant cereals (Sperling et al., BBA 1632, 1 (2003)).

Further alpha-hydroxylated saturated long chain fatty acids are cerebronic acid and (24:0; OH) and 2-Hydroxy behenic acid (22:0).

The hydroxylated fatty acids may be used in a method for producing fats or oils according to US 2003/054509 or in lanolin-free cosmetic composition according to US 2004/166130.

Whether oils with unsaturated or with saturated fatty acids are preferred depends on the intended purpose; thus, for example, lipids with unsaturated fatty acids, specifically polyunsaturated fatty acids, are preferred in human nutrition since they have a positive effect on the cholesterol level in the blood and thus on the possibility of heart disease. They are used in a variety of dietetic foodstuffs or medicaments. In addition PUFAs are commonly used in food, feed and in the cosmetic industry. Poly unsaturated w-3- and/or w-6-fatty acids are an important part of animal feed and human food. Because of the common composition of human food polyunsaturated w-3-fatty acids, which are an essential component of fish oil, should be added to the food to increase the nutritional value of the food; thus, for example, polyunsaturated fatty acids such as DHA or EPA are added as mentioned above to infant formula to increase its nutritional value. The true essential fatty acids linoleic and linolenic fatty acid have a lot of positive effects in the human body such as a positive effect on healthy heart, arteries and skin. They bring for example relieve from eczema, diabetic neuropathy or PMS and cyclical breast pain.

Further poly unsaturated w-3- and/or w-6-fatty acids important part of animal feed and human food are delta 7, 10 hexadecadienic acid (16:2(n-6)) and delta 7,10,13 hexadecatrienic acid (16:3(n-3)). Hexadecadienic acid is a minor component of some seed and fish oils, and of plant leaves but the precursor of hexadecatrienic acid 16:3(n-3), which is a common constituent of leaf lipids. This acid is known to occur in photosynthetic leaves, such as for example Arabidopsis thaliana, rape leaves, fern lipid, ginko leaves, potato leaves, tomato leaves and spinach. It may also occur in the leaves of Brassicaceae plants, such as horse radish, cabbage, turnip, Chinese mustard, cauliflower and watercress.

In higher plants, the galactolipids contain a high proportion of polyunsaturated fatty acids, up to 95% of which can be linolenic acid (18:3(n-3)). In this instance, the most abundant molecular species of mono- and digalactosyldiacylglycerols must have 18:3 at both sn-I and sn-2 positions of the glycerol backbone. Plants such as the pea, which have 18:3 as almost the only fatty acid in the monogalactosyldiacylglycerols, have been termed “18:3 plants”. Other species, and Arabidopsis thaliana is an example, contain appreciable amounts of hexadecatrienoic acid (16:3(n-3)) in the monogalactosyldiacylglycerols, and they are termed “16:3 plants”.

As mentioned, polyunsaturated fatty acid are further used in the cosmetic industry. The application US 20030039672 discloses a cosmetic method for treating aged, sensitive, dry, flaky, wrinkled and/or photodamaged skin through topical application of a composition which comprises an unsaturated C16 fatty acid having at least three double bonds, which may be preferably hexadecatrienoic acid.

It is an object of the present invention to develop an inexpensive process for the synthesis of 2-hydroxybehenic acid, 2-hydroxypalmitic acid, 2-hydroxytetracosenoic acid, (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, and/or oleic acid.

for the disclosure of this paragraph see [0013.1.8.8] above.

Accordingly, in a first embodiment, the invention relates to a process for the production of at least one (preferably a) fine chemical selected from the group consisting of: 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, and oleic acid, or, in other words, of the “fine chemical” or “fine chemical of the invention”.

The terms “fine chemical of the invention”, “fine chemical” or “the fine chemical” are used herein equally and relate in context of the paragraphs or sections [0014.1.12.12] to [0514.1.8.12] essentially to the metabolite or the metabolites indicated in column 7, application no. 12 of Tables I, II and IV in the respective line.

Further, the term “in context of any of the paragraphs [0014.1.12.12] to [0514.1.8.12]” as used herein means that for any of said paragraphs [0014.1.12.12] to [0514.1.8.12] the term “the fine chemical” is understood to follow the definition of paragraphs or sections [0014.1.12.12] and [0015.1.12.12], independently whether it refers to any other paragraph or not and whether the reference recites the term “fine chemical” in an other context.

Thus, in cases where one or more paragraphs or sections are incorporated by reference into any of the present paragraphs [0014.1.12.12] to [0514.1.8.12], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to the definition of paragraph [0014.1.12.12] and [0015.1.12.12].

Accordingly, in one embodiment, the term “fine chemical of the invention”, “fine chemical” or “the fine chemical” as used herein (alone or in combination, like in FCRP) means “2-hydroxybehenic acid” in context of the nucleic acid or polypeptide sequences listed in the respective same line of any one of Tables I to IV of application no. 12 and indicating in column 7 the metabolite “2-hydroxybehenic acid”.

In one embodiment, the term 2-hydroxybehenic acid or the term “fine chemical” mean in context of the paragraphs or sections [0014.1.12.12] to [0514.1.8.12] at least one chemical compound with an activity of the above mentioned 2-hydroxybehenic acid, respectively.

Accordingly, in one embodiment, the term “fine chemical of the invention”, “fine chemical” or “the fine chemical” as used herein (alone or in combination, like in FCRP) means “2-hydroxypalmitic acid” in context of the nucleic acid or polypeptide sequences listed in the respective same line of any one of Tables I to IV of application no. 12 and indicating in column 7 the metabolite “2-hydroxypalmitic acid”.

In one embodiment, the term 2-hydroxypalmitic acid or the term “fine chemical” mean in context of the paragraphs or sections [0014.1.12.12] to [0514.1.8.12] at least one chemical compound with an activity of the above mentioned 2-hydroxypalmitic acid, respectively.

Accordingly, in one embodiment, the term “fine chemical of the invention”, “fine chemical” or “the fine chemical” as used herein (alone or in combination, like in FCRP) means “2-hydroxytetracosenoic acid (preferably 2-hydroxynervonic acid)” in context of the nucleic acid or polypeptide sequences listed in the respective same line of any one of Tables I to IV of application no. 12 and indicating in column 7 the metabolite “2-hydroxytetracosenoic acid (preferably 2-hydroxynervonic acid)”.

In one embodiment, the term 2-hydroxytetracosenoic acid (preferably 2-hydroxynervonic acid) or the term “fine chemical” mean in context of the paragraphs or sections [0014.1.12.12] to [0514.1.8.12] at least one chemical compound with an activity of the above mentioned 2-hydroxytetracosenoic acid (preferably 2-hydroxynervonic acid), respectively.

Accordingly, in one embodiment, the term “fine chemical of the invention”, “fine chemical” or “the fine chemical” as used herein (alone or in combination, like in FCRP) means “behenic acid” in context of the nucleic acid or polypeptide sequences listed in the respective same line of any one of Tables I to IV of application no. 12 and indicating in column 7 the metabolite “behenic acid”.

In one embodiment, the term behenic acid or the term “fine chemical” mean in context of the paragraphs or sections [0014.1.12.12] to [0514.1.8.12] at least one chemical compound with an activity of the above mentioned behenic acid, respectively.

Accordingly, in one embodiment, the term “fine chemical of the invention”, “fine chemical” or “the fine chemical” as used herein (alone or in combination, like in FCRP) means “cerebronic acid” in context of the nucleic acid or polypeptide sequences listed in the respective same line of any one of Tables I to IV of application no. 12 and indicating in column 7 the metabolite “cerebronic acid”.

In one embodiment, the term cerebronic acid or the term “fine chemical” mean in context of the paragraphs or sections [0014.1.12.12] to [0514.1.8.12] at least one chemical compound with an activity of the above mentioned cerebronic acid, respectively.

Accordingly, in one embodiment, the term “fine chemical of the invention”, “fine chemical” or “the fine chemical” as used herein (alone or in combination, like in FCRP) means “eicosanoic acid” in context of the nucleic acid or polypeptide sequences listed in the respective same line of any one of Tables I to IV of application no. 12 and indicating in column 7 the metabolite “eicosanoic acid”.

In one embodiment, the term eicosanoic acid or the term “fine chemical” mean in context of the paragraphs or sections [0014.1.12.12] to [0514.1.8.12] at least one chemical compound with an activity of the above mentioned eicosanoic acid, respectively.

Accordingly, in one embodiment, the term “fine chemical of the invention”, “fine chemical” or “the fine chemical” as used herein (alone or in combination, like in FCRP) means “heptadecanoic acid” in context of the nucleic acid or polypeptide sequences listed in the respective same line of any one of Tables I to IV of application no. 12 and indicating in column 7 the metabolite “heptadecanoic acid”.

In one embodiment, the term heptadecanoic acid or the term “fine chemical” mean in context of the paragraphs or sections [0014.1.12.12] to [0514.1.8.12] at least one chemical compound with an activity of the above mentioned heptadecanoic acid, respectively.

Accordingly, in one embodiment, the term “fine chemical of the invention”, “fine chemical” or “the fine chemical” as used herein (alone or in combination, like in FCRP) means “hexadecadienoic acid” in context of the nucleic acid or polypeptide sequences listed in the respective same line of any one of Tables I to IV of application no. 12 and indicating in column 7 the metabolite “hexadecadienoic acid”.

In one embodiment, the term hexadecadienoic acid or the term “fine chemical” mean in context of the paragraphs or sections [0014.1.12.12] to [0514.1.8.12] at least one chemical compound with an activity of the above mentioned hexadecadienoic acid, respectively.

Accordingly, in one embodiment, the term “fine chemical of the invention”, “fine chemical” or “the fine chemical” as used herein (alone or in combination, like in FCRP) means “hexadecatrienoic acid” in context of the nucleic acid or polypeptide sequences listed in the respective same line of any one of Tables I to IV of application no. 12 and indicating in column 7 the metabolite “hexadecatrienoic acid”.

In one embodiment, the term hexadecatrienoic acid or the term “fine chemical” mean in context of the paragraphs or sections [0014.1.12.12] to [0514.1.8.12] at least one chemical compound with an activity of the above mentioned hexadecatrienoic acid, respectively.

Accordingly, in one embodiment, the term “fine chemical of the invention”, “fine chemical” or “the fine chemical” as used herein (alone or in combination, like in FCRP) means “lauric acid” in context of the nucleic acid or polypeptide sequences listed in the respective same line of any one of Tables I to IV of application no. 12 and indicating in column 7 the metabolite “lauric acid”.

In one embodiment, the term lauric acid or the term “fine chemical” mean in context of the paragraphs or sections [0014.1.12.12] to [0514.1.8.12] at least one chemical compound with an activity of the above mentioned lauric acid, respectively.

Accordingly, in one embodiment, the term “fine chemical of the invention”, “fine chemical” or “the fine chemical” as used herein (alone or in combination, like in FCRP) means “myristic acid” in context of the nucleic acid or polypeptide sequences listed in the respective same line of any one of Tables I to IV of application no. 12 and indicating in column 7 the metabolite “myristic acid”.

In one embodiment, the term myristic acid or the term “fine chemical” mean in context of the paragraphs or sections [0014.1.12.12] to [0514.1.8.12] at least one chemical compound with an activity of the above mentioned myristic acid, respectively.

Accordingly, in one embodiment, the term “fine chemical of the invention”, “fine chemical” or “the fine chemical” as used herein (alone or in combination, like in FCRP) means “nervonic acid” in context of the nucleic acid or polypeptide sequences listed in the respective same line of any one of Tables I to IV of application no. 12 and indicating in column 7 the metabolite “nervonic acid”.

In one embodiment, the term nervonic acid or the term “fine chemical” mean in context of the paragraphs or sections [0014.1.12.12] to [0514.1.8.12] at least one chemical compound with an activity of the above mentioned nervonic acid, respectively.

Accordingly, in one embodiment, the term “fine chemical of the invention”, “fine chemical” or “the fine chemical” as used herein (alone or in combination, like in FCRP) means “oleic acid” in context of the nucleic acid or polypeptide sequences listed in the respective same line of any one of Tables I to IV of application no. 12 and indicating in column 7 the metabolite “oleic acid”.

In one embodiment, the term oleic acid or the term “fine chemical” mean in context of the paragraphs or sections [0014.1.12.12] to [0514.1.8.12] at least one chemical compound with an activity of the above mentioned oleic acid, respectively.

Accordingly, in one embodiment, the terms “fine chemical of the invention”, “fine chemical” or “the fine chemical” mean in context of any of the paragraphs [0014.1.12.12] to [0514.1.8.12] 2-hydroxybehenic acid, its tryglycerides, lipids, oils and/or fats in free form or bound form. In a preferred embodiment, the term “the fine chemical” means 2-hydroxybehenic acid in free form or e.g. in form of its tryglycerides, lipids, oils and/or fats. In a preferred embodiment “2-hydroxybehenic acid” means 2-hydroxybehenic acid in free form.

Accordingly, in one embodiment, the terms “fine chemical of the invention”, “fine chemical” or “the fine chemical” mean in context of any of the paragraphs [0014.1.12.12] to [0514.1.8.12] 2-hydroxypalmitic acid, its tryglycerides, lipids, oils and/or fats in free form or bound form. In a preferred embodiment, the term “the fine chemical” means 2-hydroxypalmitic acid in free form or e.g. in form of its tryglycerides, lipids, oils and/or fats. In a preferred embodiment “2-hydroxypalmitic acid” means 2-hydroxypalmitic acid in free form.

Accordingly, in one embodiment, the terms “fine chemical of the invention”, “fine chemical” or “the fine chemical” mean in context of any of the paragraphs [0014.1.12.12] to [0514.1.8.12] 2-hydroxytetracosenoic acid (preferably 2-hydroxynervonic acid), its tryglycerides, lipids, oils and/or fats in free form or bound form. In a preferred embodiment, the term “the fine chemical” means 2-hydroxytetracosenoic acid (preferably 2-hydroxynervonic acid) in free form or e.g. in form of its tryglycerides, lipids, oils and/or fats. In a preferred embodiment “2-hydroxytetracosenoic acid (preferably 2-hydroxynervonic acid)” means 2-hydroxytetracosenoic acid (preferably 2-hydroxynervonic acid) in free form.

Accordingly, in one embodiment, the terms “fine chemical of the invention”, “fine chemical” or “the fine chemical” mean in context of any of the paragraphs [0014.1.12.12] to [0514.1.8.12] behenic acid, its tryglycerides, lipids, oils and/or fats in free form or bound form. In a preferred embodiment, the term “the fine chemical” means behenic acid in free form or e.g. in form of its its tryglycerides, lipids, oils and/or fats. In a preferred embodiment “behenic acid” means behenic acid in free form.

Accordingly, in one embodiment, the terms “fine chemical of the invention”, “fine chemical” or “the fine chemical” mean in context of any of the paragraphs [0014.1.12.12] to [0514.1.8.12] cerebronic acid, its tryglycerides, lipids, oils and/or fats in free form or bound form. In a preferred embodiment, the term “the fine chemical” means cerebronic acid in free form or e.g. in form of its tryglycerides, lipids, oils and/or fats. In a preferred embodiment “cerebronic acid” means cerebronic acid in free form.

Accordingly, in one embodiment, the terms “fine chemical of the invention”, “fine chemical” or “the fine chemical” mean in context of any of the paragraphs [0014.1.12.12] to [0514.1.8.12] eicosanoic acid, its tryglycerides, lipids, oils and/or fats in free form or bound form. In a preferred embodiment, the term “the fine chemical” means eicosanoic acid in free form or e.g. in form of its tryglycerides, lipids, oils and/or fats. In a preferred embodiment “eicosanoic acid” means eicosanoic acid in free form.

Accordingly, in one embodiment, the terms “fine chemical of the invention”, “fine chemical” or “the fine chemical” mean in context of any of the paragraphs [0014.1.12.12] to [0514.1.8.12] heptadecanoic acid, its tryglycerides, lipids, oils and/or fats in free form or bound form. In a preferred embodiment, the term “the fine chemical” means heptadecanoic acid in free form or e.g in form of its tryglycerides, lipids, oils and/or fats. In a preferred embodiment “heptadecanoic acid” means heptadecanoic acid in free form.

Accordingly, in one embodiment, the terms “fine chemical of the invention”, “fine chemical” or “the fine chemical” mean in context of any of the paragraphs [0014.1.12.12] to [0514.1.8.12] hexadecadienoic acid, its tryglycerides, lipids, oils and/or fats in free form or bound form. In a preferred embodiment, the term “the fine chemical” means hexadecadienoic acid in free form or e.g. in form of its tryglycerides, lipids, oils and/or fats. In a preferred embodiment “hexadecadienoic acid” means hexadecadienoic acid in free form.

Accordingly, in one embodiment, the terms “fine chemical of the invention”, “fine chemical” or “the fine chemical” mean in context of any of the paragraphs [0014.1.12.12] to [0514.1.8.12] hexadecatrienoic acid, its tryglycerides, lipids, oils and/or fats in free form or bound form. In a preferred embodiment, the term “the fine chemical” means hexadecatrienoic acid in free form e.g. in form of its tryglycerides, lipids, oils and/or fats. In a preferred embodiment “hexadecatrienoic acid” means hexadecatrienoic acid in free form.

Accordingly, in one embodiment, the terms “fine chemical of the invention”, “fine chemical” or “the fine chemical” mean in context of any of the paragraphs [0014.1.12.12] to [0514.1.8.12] lauric acid, its tryglycerides, lipids, oils and/or fats in free form or bound form. In a preferred embodiment, the term “the fine chemical” means lauric acid in free form or e.g. in form of its tryglycerides, lipids, oils and/or fats. In a preferred embodiment “lauric acid” means lauric acid in free form.

Accordingly, in one embodiment, the terms “fine chemical of the invention”, “fine chemical” or “the fine chemical” mean in context of any of the paragraphs [0014.1.12.12] to [0514.1.8.12] myristic acid, its tryglycerides, lipids, oils and/or fats in free form or bound form. In a preferred embodiment, the term “the fine chemical” means myristic acid in free form or e.g. in form of its tryglycerides, lipids, oils and/or fats. In a preferred embodiment “myristic acid” means myristic acid in free form.

Accordingly, in one embodiment, the terms “fine chemical of the invention”, “fine chemical” or “the fine chemical” mean in context of any of the paragraphs [0014.1.12.12] to [0514.1.8.12] nervonic acid, its tryglycerides, lipids, oils and/or fats in free form or bound form. In a preferred embodiment, the term “the fine chemical” means nervonic acid in free form or e.g. in form of its tryglycerides, lipids, oils and/or fats. In a preferred embodiment “nervonic acid” means nervonic acid in free form.

Accordingly, in one embodiment, the terms “fine chemical of the invention”, “fine chemical” or “the fine chemical” mean in context of any of the paragraphs [0014.1.12.12] to [0514.1.8.12] oleic acid, its tryglycerides, lipids, oils and/or fats in free form or bound form. In a preferred embodiment, the term “the fine chemical” means oleic acid in free form or e.g. in form of its tryglycerides, lipids, oils and/or fats. In a preferred embodiment “oleic acid” means oleic acid in free form.

In one embodiment, the term “the fine chemical” or “the respective fine chemical” mean in context of any of the paragraphs [0014.1.12.12] to [0514.1.8.12] 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, and/or oleic acid, respectively and/or tryglycerides, lipids, oils and/or fats of 2-hydroxybehenic acid, 2-hydroxypalmitic acid, 2-hydroxytetracosenoic acid(preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, and/or oleic acid, respectively and its salts, ester, thioester or 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, and/or oleic acid in free form or bound to other compounds such as triglycerides, glycolipids, phospholipids etc. In a preferred embodiment, the term “the fine chemical” means 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, and/or oleic acid., respectively, in free form or its salts or bound to triglycerides. Triglycerides, lipids, oils, fats or lipid mixture thereof shall mean any triglyceride, lipid, oil and/or fat containing any bound or free 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, and/or oleic acid, respectively, for example sphingolipids, phosphoglycerides, lipids, glycolipids such as glycosphingolipids, phospholipids such as phosphatidylethanolamine, phosphatidylcholine, phosphatidylserine, phosphatidylglycerol, phosphatidylinositol or diphosphatidylglycerol, or as monoacylglyceride, diacylglyceride or triacylglyceride or other fatty acid esters such as acetyl-Coenzym A thioester, which contain further saturated or unsaturated fatty acids in the fatty acid molecule.

In one embodiment, the term “the fine chemical” and the term “the respective fine chemical” mean at least one chemical compound with an activity of the abovementioned fine chemical. Further, the term “in context of any of the paragraphs [0014.1.12.12] to [0514.1.8.12]” as used herein means that for any of said paragraphs [0014.1.12.12] to [0514.1.8.12] the term “the fine chemical” is understood to follow the definition of section [0014.1.12.12] or section [0015.1.12.12], independently whether it refers to any other paragraph or not and whether the reference recites the term “fine chemical” in an other context.

Thus, in cases where one or more paragraphs or sections are incorporated by reference into any of the present paragraphs [0014.1.12.12] to [0514.1.8.12], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to this definition of this paragraph [0015.1.12.12].

Further, the term “fine chemicals” as used herein relates to compositions comprising said fine chemical(s), i.e. comprising 2-hydroxybehenic acid, 2-hydroxypalmitic acid, 2-hydroxytetracosenoic acid(preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, and/or oleic acid, respectively.

Accordingly, the present invention relates to a process for the production of cerebronic acid, which comprises
(a) increasing or generating one or more activities selected from the group consisting of chlorophyllase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of chlorophyllase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human

Accordingly, the present invention relates to a process for the production of hexadecatrienoic acid, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of cerebronic acid, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of hexadecatrienoic acid, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxybehenic acid, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxypalmitic acid, which comprises
(a) increasing or generating one or more activities selected from the group consisting of At2g33510-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of cerebronic acid, which comprises
(a) increasing or generating one or more activities selected from the group consisting of adenosine kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of 2-hydroxybehenic acid, which comprises
(a) increasing or generating one or more activities selected from the group consisting of adenosine kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of DNA mismatch repair protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of cerebronic acid, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of hexadecatrienoic acid, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxybehenic acid, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxypalmitic acid, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of 2-hydroxybehenic acid, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxypalmitic acid, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of hexadecatrienoic acid, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxybehenic acid, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of cerebronic acid, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of 2-hydroxybehenic acid, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of cerebronic acid, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of hexadecadienoic acid, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecatrienoic acid, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxybehenic acid, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxypalmitic acid, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of oleic acid, which comprises
(a) increasing or generating one or more activities selected from the group consisting of acyldesaturase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

Accordingly, the present invention relates to a process for the production of hexadecatrienoic acid, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of hexadecatrienoic acid, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of hexadecadienoic acid, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of cerebronic acid, which comprises
(a) increasing or generating one or more activities selected from the group consisting of CDP-diacylglycerol-serine O-phosphatidyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of 2-hydroxybehenic acid, which comprises
(a) increasing or generating one or more activities selected from the group consisting of

CDP-diacylglycerol-serine O-phosphatidyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of CDP-diacylglycerol-serine O-phosphatidyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxypalmitic acid, which comprises
(a) increasing or generating one or more activities selected from the group consisting of CDP-diacylglycerol-serine O-phosphatidyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of hexadecadienoic acid, which comprises
(a) increasing or generating one or more activities selected from the group consisting of sec-independent protein translocase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of transcriptional regulator protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human

Accord ivp,iAhre present invention relates to a process for the production of 2-hydroxypalmitic acid, which comprises
(a) increasing or generating one or more activities selected from the group consisting of beta-hydroxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of hexadecadienoic acid, which comprises
(a) increasing or generating one or more activities selected from the group consisting of 30S ribosomal protein S3 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of 2-hydroxybehenic acid, which comprises
(a) increasing or generating one or more activities selected from the group consisting of acyl-CoA synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of heat shock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human

AccordirgjiNAhre present invention relates to a process for the production of 2-hydroxytetracosenoic acid (preferably 2-hydroxynervonic acid), which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of hexadecadienoic acid, which comprises
(a) increasing or generating one or more activities selected from the group consisting of recombinase A in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of lauric acid, which comprises
(a) increasing or generating one or more activities selected from the group consisting of ABC transporter component in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lauric acid or a composition comprising lauric acid in said non-human organism or in the culture medium surrounding said non-human organism.

Accordingly, the present invention relates to a process for the production of 2-hydroxypalmitic acid, which comprises
(a) increasing or generating one or more activities selected from the group consisting of hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of 2-hydroxypalmitic acid, which comprises
(a) increasing or generating one or more activities selected from the group consisting of glutamate-ammonia-ligase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of 2-hydroxybehenic acid, which comprises
(a) increasing or generating one or more activities selected from the group consisting of fumarylacetoacetate hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of N-acetyl-gamma-glutamyl-phosphate reductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of HesB/YadR/YfhF family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human Accord ivp,iAhre present invention relates to a process for the production of 2-hydroxytetracosenoic acid (preferably 2-hydroxynervonic acid), which comprises
(a) increasing or generating one or more activities selected from the group consisting of purine nucleoside phosphorylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human Accord ivp,iAhre present invention relates to a process for the production of 2-hydroxybehenic acid, which comprises
(a) increasing or generating one or more activities selected from the group consisting of b0050-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of b0050-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of cerebronic acid, which comprises
(a) increasing or generating one or more activities selected from the group consisting of b0249-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of behenic acid, which comprises
(a) increasing or generating one or more activities selected from the group consisting of betaine aldehyde dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of behenic acid or a composition comprising behenic acid in said non-human organism or in the culture medium surrounding said non-human organism.

Accordingly, the present invention relates to a process for the production of eicosanoic acid, which comprises
(a) increasing or generating one or more activities selected from the group consisting of betaine aldehyde dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of eicosanoic acid or a composition comprising eicosanoic acid in said non-human organism or in the culture medium surrounding said non-human organism.

Accordingly, the present invention relates to a process for the production of myristic acid, which comprises
(a) increasing or generating one or more activities selected from the group consisting of 2,3-dihydroxyphenylpropionate 1,2-dioxygenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myristic acid or a composition comprising myristic acid in said non-human organism or in the culture medium surrounding said non-human organism.

Accordingly, the present invention relates to a process for the production of oleic acid, which comprises
(a) increasing or generating one or more activities selected from the group consisting of 2,3-dihydroxyphenylpropionate 1,2-dioxygenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

Accordingly, the present invention relates to a process for the production of hexadecatrienoic acid, which comprises
(a) increasing or generating one or more activities selected from the group consisting of b0371-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of cerebronic acid, which comprises
(a) increasing or generating one or more activities selected from the group consisting of ATP-binding component of a transport system in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of 2-hydroxybehenic acid, which comprises
(a) increasing or generating one or more activities selected from the group consisting of ATP-binding component of a transport system in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxypalmitic acid, which comprises
(a) increasing or generating one or more activities selected from the group consisting of ATP-binding component of a transport system in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of hexadecadienoic acid, which comprises
(a) increasing or generating one or more activities selected from the group consisting of b0518-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of 2-hydroxybehenic acid, which comprises
(a) increasing or generating one or more activities selected from the group consisting of sensor histidine kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of sensor histidine kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human Accord ivp,iAhre present invention relates to a process for the production of cerebronic acid, which comprises
(a) increasing or generating one or more activities selected from the group consisting of citrate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of b0801-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human Accordirgjip,iAhre present invention relates to a process for the production of cerebronic acid, which comprises
(a) increasing or generating one or more activities selected from the group consisting of b1046-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of hexadecatrienoic acid, which comprises
(a) increasing or generating one or more activities selected from the group consisting of malonyl CoA-acyl carrier protein transacylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of hexadecadienoic acid, which comprises
(a) increasing or generating one or more activities selected from the group consisting of 3-oxoacyl-[acyl-carrier-protein] reductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecatrienoic acid, which comprises
(a) increasing or generating one or more activities selected from the group consisting of 3-oxoacyl-[acyl-carrier-protein] reductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of hexadecatrienoic acid, which comprises
(a) increasing or generating one or more activities selected from the group consisting of 3-oxoacyl-[acyl-carrier-protein] synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxybehenic acid, which comprises
(a) increasing or generating one or more activities selected from the group consisting of 3-oxoacyl-[acyl-carrier-protein] synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of oleic acid, which comprises
(a) increasing or generating one or more activities selected from the group consisting of sodium/proton antiporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

Accordingly, the present invention relates to a process for the production of 2-hydroxypalmitic acid, which comprises
(a) increasing or generating one or more activities selected from the group consisting of sodium/proton antiporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of behenic acid, which comprises
(a) increasing or generating one or more activities selected from the group consisting of outer-membrane lipoprotein precursor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of behenic acid or a composition comprising behenic acid in said non-human organism or in the culture medium surrounding said non-human organism.

Accordingly, the present invention relates to a process for the production of behenic acid, which comprises
(a) increasing or generating one or more activities selected from the group consisting of cardiolipin synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of behenic acid or a composition comprising behenic acid in said non-human organism or in the culture medium surrounding said non-human organism.

Accordingly, the present invention relates to a process for the production of heptadecanoic acid, which comprises
(a) increasing or generating one or more activities selected from the group consisting of cardiolipin synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of heptadecanoic acid or a composition comprising heptadecanoic acid in said non-human organism or in the culture medium surrounding said non-human organism.

Accordingly, the present invention relates to a process for the production of hexadecatrienoic acid, which comprises
(a) increasing or generating one or more activities selected from the group consisting of b1346-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of lauric acid, which comprises
(a) increasing or generating one or more activities selected from the group consisting of phage-related repressor protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lauric acid or a composition comprising lauric acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of lipoprotein precursor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of hexadecatrienoic acid, which comprises
(a) increasing or generating one or more activities selected from the group consisting of b1470-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of hexadecatrienoic acid, which comprises
(a) increasing or generating one or more activities selected from the group consisting of NADP-dependent malic enzyme in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxybehenic acid, which comprises
(a) increasing or generating one or more activities selected from the group consisting of b1522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of hexadecatrienoic acid, which comprises
(a) increasing or generating one or more activities selected from the group consisting of acid shock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of phosphotransferase system component in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxybehenic acid, which comprises
(a) increasing or generating one or more activities selected from the group consisting of electron transport complex protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of hexadecatrienoic acid, which comprises
(a) increasing or generating one or more activities selected from the group consisting of pyridoxamine kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of b1670-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, tre present invention relates to a process for the production of hexadecadienoic acid, which comprises
(a) increasing or generating one or more activities selected from the group consisting of b1703-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecatrienoic acid, which comprises
(a) increasing or generating one or more activities selected from the group consisting of b1703-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of hexadecadienoic acid, which comprises
(a) increasing or generating one or more activities selected from the group consisting of transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecadienoic acid, which comprises
(a) increasing or generating one or more activities selected from the group consisting of b1837-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecatrienoic acid, which comprises
(a) increasing or generating one or more activities selected from the group consisting of b1837-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of hexadecadienoic acid, which comprises
(a) increasing or generating one or more activities selected from the group consisting of b2032-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecadienoic acid, which comprises
(a) increasing or generating one or more activities selected from the group consisting of b2099-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecatrienoic acid, which comprises
(a) increasing or generating one or more activities selected from the group consisting of b2099-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of b2107-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of lauric acid, which comprises
(a) increasing or generating one or more activities selected from the group consisting of b2126-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lauric acid or a composition comprising lauric acid in said non-human organism or in the culture medium surrounding said non-human organism.

Accordingly, the present invention relates to a process for the production of 2-hydroxypalmitic acid, which comprises
(a) increasing or generating one or more activities selected from the group consisting of b2139-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of hexadecatrienoic acid, which comprises
(a) increasing or generating one or more activities selected from the group consisting of ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxybehenic acid, which comprises
(a) increasing or generating one or more activities selected from the group consisting of sensor kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of b2360-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of cysteine synthase A in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of hexadecadienoic acid, which comprises
(a) increasing or generating one or more activities selected from the group consisting of ethanolamine utilization protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecatrienoic acid, which comprises
(a) increasing or generating one or more activities selected from the group consisting of ethanolamine utilization protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of oleic acid, which comprises
(a) increasing or generating one or more activities selected from the group consisting of ethanolamine utilization protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

Accordingly, the present invention relates to a process for the production of cerebronic acid, which comprises
(a) increasing or generating one or more activities selected from the group consisting of b2474-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of hexadecatrienoic acid, which comprises
(a) increasing or generating one or more activities selected from the group consisting of b2474-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of b2474-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human

Accord ivp,iAhre present invention relates to a process for the production of hexadecadienoic acid, which comprises
(a) increasing or generating one or more activities selected from the group consisting of b2513-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecadienoic acid, which comprises
(a) increasing or generating one or more activities selected from the group consisting of phosphatidylserine synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of b2654-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of hexadecatrienoic acid, which comprises
(a) increasing or generating one or more activities selected from the group consisting of 2-C-methyl-D-erythritol 4-phosphate cytidylyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of eicosanoic acid, which comprises
(a) increasing or generating one or more activities selected from the group consisting of alkaline phosphatase isozyme conversion protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of eicosanoic acid or a composition comprising eicosanoic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of murein transglycosylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of hexadecadienoic acid, which comprises
(a) increasing or generating one or more activities selected from the group consisting of b3442-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of cerebronic acid, which comprises
(a) increasing or generating one or more activities selected from the group consisting of sugar transport system permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of 2-hydroxybehenic acid, which comprises
(a) increasing or generating one or more activities selected from the group consisting of b3812-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of hexadecadienoic acid, which comprises
(a) increasing or generating one or more activities selected from the group consisting of 3-ketoacyl-CoA thiolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of oleic acid, which comprises
(a) increasing or generating one or more activities selected from the group consisting of 3-ketoacyl-CoA thiolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

Accordingly, the present invention relates to a process for the production of hexadecadienoic acid, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecadienoic acid, which comprises
(a) increasing or generating one or more activities selected from the group consisting of fructose 1,6-bisphosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of behenic acid, which comprises
(a) increasing or generating one or more activities selected from the group consisting of cystathionine gamma-synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of behenic acid or a composition comprising behenic acid in said non-human organism or in the culture medium surrounding said non-human organism.

Accordingly, the present invention relates to a process for the production of heptadecanoic acid, which comprises
(a) increasing or generating one or more activities selected from the group consisting of cystathionine gamma-synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of heptadecanoic acid or a composition comprising heptadecanoic acid in said non-human organism or in the culture medium surrounding said non-human organism.

Accordingly, the present invention relates to a process for the production of oleic acid, which comprises
(a) increasing or generating one or more activities selected from the group consisting of cystathionine gamma-synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

Accordingly, the present invention relates to a process for the production of 2-hydroxypalmitic acid, which comprises
(a) increasing or generating one or more activities selected from the group consisting of b3944-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of 2-hydroxybehenic acid, which comprises
(a) increasing or generating one or more activities selected from the group consisting of ankyrin repeat protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of hexadecadienoic acid, which comprises
(a) increasing or generating one or more activities selected from the group consisting of b4023-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of heptadecanoic acid, which comprises
(a) increasing or generating one or more activities selected from the group consisting of acetyl-coenzyme A synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of heptadecanoic acid or a composition comprising heptadecanoic acid in said non-human organism or in the culture medium surrounding said non-human organism.

Accordingly, the present invention relates to a process for the production of 2-hydroxypalmitic acid, which comprises
(a) increasing or generating one or more activities selected from the group consisting of b4205-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of 2-hydroxybehenic acid, which comprises
(a) increasing or generating one or more activities selected from the group consisting of vacuolar processing enzyme in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxypalmitic acid, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of cerebronic acid, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of hexadecatrienoic acid, which comprises P1 (a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of GM02LC38418-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of hexadecadienoic acid, which comprises
(a) increasing or generating one or more activities selected from the group consisting of sll0281-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of bifunctional protein (phosphoribosyltransferase and regulatory protein) in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of sll0406-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of cerebronic acid, which comprises
(a) increasing or generating one or more activities selected from the group consisting of binding-protein-dependent transport systems inner membrane component in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of cerebronic acid, which comprises
(a) increasing or generating one or more activities selected from the group consisting of phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of hexadecadienoic acid, which comprises
(a) increasing or generating one or more activities selected from the group consisting of carbon dioxide concentrating mechanism protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecatrienoic acid, which comprises
(a) increasing or generating one or more activities selected from the group consisting of carbon dioxide concentrating mechanism protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of cerebronic acid, which comprises
(a) increasing or generating one or more activities selected from the group consisting of coproporphyrinogen III oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of coproporphyrinogen III oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxypalmitic acid, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of hexadecatrienoic acid, which comprises
(a) increasing or generating one or more activities selected from the group consisting of bifunctional purine biosynthesis protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of cerebronic acid, which comprises
(a) increasing or generating one or more activities selected from the group consisting of malate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of hexadecatrienoic acid, which comprises
(a) increasing or generating one or more activities selected from the group consisting of glycine cleavage system H protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of permease protein of ABC transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of hexadecadienoic acid, which comprises
(a) increasing or generating one or more activities selected from the group consisting of tryptophan synthase alpha chain in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of 2-hydroxybehenic acid, which comprises
(a) increasing or generating one or more activities selected from the group consisting of dihydrolipoamide dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxypalmitic acid, which comprises
(a) increasing or generating one or more activities selected from the group consisting of phosphate import ATP-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of hexadecatrienoic acid, which comprises
(a) increasing or generating one or more activities selected from the group consisting of glutamate 5-kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of cerebronic acid, which comprises
(a) increasing or generating one or more activities selected from the group consisting of threonine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of hexadecatrienoic acid, which comprises
(a) increasing or generating one or more activities selected from the group consisting of threonine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of threonine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of ferredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxybehenic acid, which comprises
(a) increasing or generating one or more activities selected from the group consisting of Sec-independent protein translocase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of amino acid ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of cerebronic acid, which comprises
(a) increasing or generating one or more activities selected from the group consisting of metal-dependent hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of hexadecadienoic acid, which comprises
(a) increasing or generating one or more activities selected from the group consisting of metal-dependent hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of 2-hydroxybehenic acid, which comprises
(a) increasing or generating one or more activities selected from the group consisting of multiple antibiotic resistance protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of hexadecatrienoic acid, which comprises
(a) increasing or generating one or more activities selected from the group consisting of TTC1386-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxybehenic acid, which comprises
(a) increasing or generating one or more activities selected from the group consisting of homocitrate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of hexadecadienoic acid, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecatrienoic acid, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of hexadecadienoic acid, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecatrienoic acid, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of hexadecatrienoic acid, which comprises
(a) increasing or generating one or more activities selected from the group consisting of cell division control protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of cerebronic acid, which comprises
(a) increasing or generating one or more activities selected from the group consisting of ycl027w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of ycl027w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxypalmitic acid, which comprises
(a) increasing or generating one or more activities selected from the group consisting of ycl049c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of oleic acid, which comprises
(a) increasing or generating one or more activities selected from the group consisting of glycerol-3-phosphate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

Accordingly, the present invention relates to a process for the production of eicosanoic acid, which comprises
(a) increasing or generating one or more activities selected from the group consisting of isocitrate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of eicosanoic acid or a composition comprising eicosanoic acid in said non-human organism or in the culture medium surrounding said non-human organism.

Accordingly, the present invention relates to a process for the production of heptadecanoic acid, which comprises
(a) increasing or generating one or more activities selected from the group consisting of isocitrate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of heptadecanoic acid or a composition comprising heptadecanoic acid in said non-human organism or in the culture medium surrounding said non-human organism.

Accordingly, the present invention relates to a process for the production of hexadecadienoic acid, which comprises
(a) increasing or generating one or more activities selected from the group consisting of cell division control protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecatrienoic acid, which comprises
(a) increasing or generating one or more activities selected from the group consisting of cell division control protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxybehenic acid, which comprises
(a) increasing or generating one or more activities selected from the group consisting of homocitrate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of behenic acid, which comprises
(a) increasing or generating one or more activities selected from the group consisting of phospho-2-dehydro-3-deoxyheptonate aldolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of behenic acid or a composition comprising behenic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of cell wall protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of F-box protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of cerebronic acid, which comprises
(a) increasing or generating one or more activities selected from the group consisting of ydr273w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of ydr273w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human Accordirgjip,iAhre present invention relates to a process for the production of hexadecatrienoic acid, which comprises
(a) increasing or generating one or more activities selected from the group consisting of ydr339c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of heptadecanoic acid, which comprises
(a) increasing or generating one or more activities selected from the group consisting of aldolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of heptadecanoic acid or a composition comprising heptadecanoic acid in said non-human organism or in the culture medium surrounding said non-human organism.

Accordingly, the present invention relates to a process for the production of myristic acid, which comprises
(a) increasing or generating one or more activities selected from the group consisting of small nuclear ribonucleoprotein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myristic acid or a composition comprising myristic acid in said non-human organism or in the culture medium surrounding said non-human organism.

Accordingly, the present invention relates to a process for the production of hexadecadienoic acid, which comprises
(a) increasing or generating one or more activities selected from the group consisting of molecular chaperone portein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecatrienoic acid, which comprises
(a) increasing or generating one or more activities selected from the group consisting of molecular chaperone portein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxybehenic acid, which comprises
(a) increasing or generating one or more activities selected from the group consisting of YFL019C-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxypalmitic acid, which comprises
(a) increasing or generating one or more activities selected from the group consisting of YFL019C-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of hexadecadienoic acid, which comprises
(a) increasing or generating one or more activities selected from the group consisting of aminotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecatrienoic acid, which comprises
(a) increasing or generating one or more activities selected from the group consisting of ygl237c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of eicosanoic acid, which comprises
(a) increasing or generating one or more activities selected from the group consisting of YGR104C-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of eicosanoic acid or a composition comprising eicosanoic acid in said non-human organism or in the culture medium surrounding said non-human organism.

Accordingly, the present invention relates to a process for the production of cerebronic acid, which comprises
(a) increasing or generating one or more activities selected from the group consisting of squalene monooxygenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of hexadecadienoic acid, which comprises
(a) increasing or generating one or more activities selected from the group consisting of biotin synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of cerebronic acid, which comprises
(a) increasing or generating one or more activities selected from the group consisting of yhr033w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of cerebronic acid, which comprises
(a) increasing or generating one or more activities selected from the group consisting of DnaJ-like chaperone in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of 2-hydroxybehenic acid, which comprises
(a) increasing or generating one or more activities selected from the group consisting of YJL127W-A-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of cystathionine gamma-synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxypalmitic acid, which comprises
(a) increasing or generating one or more activities selected from the group consisting of cystathionine gamma-synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of 2-hydroxypalmitic acid, which comprises
(a) increasing or generating one or more activities selected from the group consisting of polygalacturonase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of hexadecatrienoic acid, which comprises
(a) increasing or generating one or more activities selected from the group consisting of cyclin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of hexadecadienoic acid, which comprises
(a) increasing or generating one or more activities selected from the group consisting of ylr326w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecatrienoic acid, which comprises
(a) increasing or generating one or more activities selected from the group consisting of ylr326w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxybehenic acid, which comprises
(a) increasing or generating one or more activities selected from the group consisting of yml083c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of yml084w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxypalmitic acid, which comprises
(a) increasing or generating one or more activities selected from the group consisting of molecular chaperone in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of oleic acid, which comprises
(a) increasing or generating one or more activities selected from the group consisting of phosphotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

Accordingly, the present invention relates to a process for the production of 2-hydroxybehenic acid, which comprises
(a) increasing or generating one or more activities selected from the group consisting of ynl321w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of hexadecatrienoic acid, which comprises
(a) increasing or generating one or more activities selected from the group consisting of Elongation factor Tu in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating one or more activities selected from the group consisting of ATPase epsilon subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxypalmitic acid, which comprises
(a) increasing or generating one or more activities selected from the group consisting of phosphoadenosine phosphosulfate reductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of oleic acid, which comprises
(a) increasing or generating one or more activities selected from the group consisting of 40S ribosomal protein S17 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

Accordingly, the present invention relates to a process for the production of 2-hydroxybehenic acid, which comprises
(a) increasing or generating one or more activities selected from the group consisting of b3644-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of behenic acid, which comprises
(a) increasing or generating one or more activities selected from the group consisting of b3644-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of behenic acid or a composition comprising behenic acid in said non-human organism or in the culture medium surrounding said non-human organism.

Accordingly, the present invention relates to a process for the production of nervonic acid, which comprises
(a) increasing or generating one or more activities selected from the group consisting of b3644-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of nervonic acid or a composition comprising nervonic acid in said non-human organism or in the culture medium surrounding said non-human organism.

An embodiment of the present invention relates to a process for the production of 2-hydroxybehenic acid, which comprises
(a1) increasing or generating the activity of a polypeptide comprising a polypeptide as depicted in a respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical 2-hydroxybehenic acid; or
(a2) increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical 2-hydroxybehenic acid; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of 2-hydroxybehenic acid or a composition comprising 2-hydroxybehenic acid in said non-human organism or in the culture medium surrounding said non-human organism.

An embodiment of the present invention relates to a process for the production of 2-hydroxypalmitic acid, which comprises
(a1) increasing or generating the activity of a polypeptide comprising a polypeptide as depicted in a respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical 2-hydroxypalmitic acid; or
(a2) increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical 2-hydroxypalmitic acid; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of 2-hydroxypalmitic acid or a composition comprising 2-hydroxypalmitic acid in said non-human organism or in the culture medium surrounding said non-human organism.

An embodiment of the present invention relates to a process for the production of 2-hydroxytetracosenoic acid (preferably 2-hydroxynervonic acid), which comprises
(a1) increasing or generating the activity of a polypeptide comprising a polypeptide as depicted in a respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical 2-hydroxytetracosenoic acid (preferably 2-hydroxynervonic acid); or
(a2) increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical 2-hydroxytetracosenoic acid (preferably 2-hydroxynervonic acid); or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

An embodiment of the present invention relates to a process for the production of behenic acid, which comprises
(a1) increasing or generating the activity of a polypeptide comprising a polypeptide as depicted in a respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical behenic acid; or
(a2) increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical behenic acid; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of behenic acid or a composition comprising behenic acid in said non-human organism or in the culture medium surrounding said non-human organism.

An embodiment of the present invention relates to a process for the production of cerebronic acid, which comprises
(a1) increasing or generating the activity of a polypeptide comprising a polypeptide as depicted in a respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical cerebronic acid; or
(a2) increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical cerebronic acid; or
(a3) increasing or generating the activity of a functional equivalent of (a1) or (a2); in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of cerebronic acid or a composition comprising cerebronic acid in said non-human organism or in the culture medium surrounding said non-human organism.

An embodiment of the present invention relates to a process for the production of eicosanoic acid, which comprises
(a1) increasing or generating the activity of a polypeptide comprising a polypeptide as depicted in a respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical eicosanoic acid; or
(a2) increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical eicosanoic acid; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of eicosanoic acid or a composition comprising eicosanoic acid in said non-human organism or in the culture medium surrounding said non-human organism.

An embodiment of the present invention relates to a process for the production of heptadecanoic acid, which comprises
(a1) increasing or generating the activity of a polypeptide comprising a polypeptide as depicted in a respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical heptadecanoic acid; or
(a2) increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical heptadecanoic acid; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of heptadecanoic acid or a composition comprising heptadecanoic acid in said non-human organism or in the culture medium surrounding said non-human organism. An embodiment of the present invention relates to a process for the production of hexadecadienoic acid, which comprises
(a1) increasing or generating the activity of a polypeptide comprising a polypeptide as depicted in a respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical hexadecadienoic acid; or
(a2) increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical hexadecadienoic acid; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of hexadecadienoic acid or a composition comprising hexadecadienoic acid in said non-human organism or in the culture medium surrounding said non-human organism. An embodiment of the present invention relates to a process for the production of hexadecatrienoic acid, which comprises
(a1) increasing or generating the activity of a polypeptide comprising a polypeptide as depicted in a respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical hexadecatrienoic acid; or
(a2) increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical hexadecatrienoic acid; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of hexadecatrienoic acid or a composition comprising hexadecatrienoic acid in said non-human organism or in the culture medium surrounding said non-human organism.

An embodiment of the present invention relates to a process for the production of lauric acid, which comprises
(a1) increasing or generating the activity of a polypeptide comprising a polypeptide as depicted in a respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical lauric acid; or
(a2) increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical lauric acid; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of lauric acid or a composition comprising lauric acid in said non-human organism or in the culture medium surrounding said non-human organism.

An embodiment of the present invention relates to a process for the production of myristic acid, which comprises
(a1) increasing or generating the activity of a polypeptide comprising a polypeptide as depicted in a respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical myristic acid; or
(a2) increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical myristic acid; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of myristic acid or a composition comprising myristic acid in said non-human organism or in the culture medium surrounding said non-human organism.

An embodiment of the present invention relates to a process for the production of nervonic acid, which comprises
(a1) increasing or generating the activity of a polypeptide comprising a polypeptide as depicted in a respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical nervonic acid; or
(a2) increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical nervonic acid; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of nervonic acid or a composition comprising nervonic acid in said non-human organism or in the culture medium surrounding said non-human organism.

An embodiment of the present invention relates to a process for the production of oleic acid, which comprises
(a1) increasing or generating the activity of a polypeptide comprising a polypeptide as depicted in a respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical oleic acid; or
(a2) increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical oleic acid; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of oleic acid or a composition comprising oleic acid in said non-human organism or in the culture medium surrounding said non-human organism.

Accordingly, the increase or generation of one or more said activities is for example conferred by one or more expression products of said nucleic acid molecule, e.g. proteins. Accordingly, in the present invention described above, the increase or generation of one or more said activities is for example conferred by one or more protein(s) each comprising a polypeptide selected from the group as depicted in Table II, application no. 12, column 5 and 8, or a homolog or a fragment thereof.

The process of the invention comprises in one embodiment the following steps:
(i) increasing or generating of the expression of; and/or
(ii) increasing or generating the expression of an expression product of; and/or
(iii) increasing or generating one or more activities of an expression product encoded by; at least one nucleic acid molecule (in the following “Fine Chemical Related Protein (FCRP)”-encoding gene or “FCRP”-gene) comprising a nucleic acid molecule selected from the group consisting of:
(a) a nucleic acid molecule encoding the polypeptide shown in column 5 or 8 of Table II, preferably Table II B, application no. 12, or a homolog or a fragment thereof;
(b) a nucleic acid molecule shown in column 5 or 8 of Table I, preferably Table I B, application no. 12, or a homolog or a fragment thereof (preferably the coding region thereof);
(c) a nucleic acid molecule, which, as a result of the degeneracy of the genetic code, can be derived from a polypeptide sequence depicted in column 5 or 8 of Table II, preferably Table II B, application no. 12;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 12, or the coding region thereof;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d),
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 12;
(i) a nucleic acid molecule encoding a polypeptide having the activity represented by a respective protein as depicted in column 5 of Table II, application no. 12;
(j) a nucleic acid molecule which comprises a polynucleotide, which is obtained by amplifying a cDNA library or a genomic library using the primers as depicted in column 8 of Table III, application no. 12; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

The term “increasing or generating of the expression of” may also be interchanged by the term “overexpressing”.

In a preferred embodiment thereof said nucleic acid molecule encodes a polypeptide which has the activity of the polypeptide represented by a protein comprising a polypeptide as depicted in the corresponding hit in column 5 of Table II, application no. 12.

In another preferred embodiment thereof said nucleic acid molecule confers a generation or an increase of the respective fine chemical in a non-human organism or a part thereof as compared to a corresponding wild-type non-human organism or part thereof.

In another preferred embodiment thereof said nucleic acid molecule encodes a polypeptide which has the activity of the polypeptide represented by a protein as depicted in the corresponding hit in column 5 of Table II, application no. 12, and confers a generation or an increase of the respective fine chemical in a non-human organism or a part thereof as compared to a corresponding wild-type non-human organism or part thereof.

Accordingly, the genes of the present invention or used in accordance with the present invention, which respectively encode a protein having an activity of 2,3-dihydroxyphenylpropionate 1,2-dioxygenase, 2-C-methyl-D-erythritol 4-phosphate cytidylyltransferase, 30S ribosomal protein S3, 3-ketoacyl-CoA thiolase, 3-oxoacyl-[acyl-carrier-protein] reductase, 3-oxoacyl-[acyl-carrier-protein] synthase, 40S ribosomal protein S17, ABC transporter component, ABC transporter permease protein, acetyl-coenzyme A synthetase, acid shock protein, acyl-CoA synthase, acyl-desaturase, adenosine kinase, aldolase, alkaline phosphatase isozyme conversion protein, amino acid ABC transporter permease protein, aminotransferase, ankyrin repeat protein, At2g33510-protein, ATPase epsilon subunit, ATP-binding component of a transport system, b0050-protein, b0249-protein, b0371-protein, b0518-protein, b0801-protein, b1046-protein, b1346-protein, b1470-protein, b1522-protein, b1670-protein, b1703-protein, b1837-protein, b2032-protein, b2099-protein, b2107-protein, b2126-protein, b2139-protein, b2360-protein, b2474-protein, b2513-protein, b2654-protein, b3442-protein, b3644-protein, b3812-protein, b3944-protein, b4023-protein, b4205-protein, beta-hydroxylase, betaine aldehyde dehydrogenase, bifunctional protein (phosphoribosyltransferase and regulatory protein), bifunctional purine biosynthesis protein, binding-protein-dependent transport systems inner membrane component, biotin synthase, calcium-dependent protein kinase, carbon dioxide concentrating mechanism protein, cardiolipin synthase, CDP-diacylglycerol-serine O-phosphatidyltransferase, cell division control protein, cell wall protein, chlorophyllase, citrate synthase, coproporphyrinogen III oxidase, cyclin, cystathionine gamma-synthase, cysteine synthase A, dihydrolipoamide dehydrogenase, DNA mismatch repair protein, DnaJ-like chaperone, electron transport complex protein, Elongation factor Tu, ethanolamine utilization protein, F-box protein, ferredoxin, fructose 1,6-bisphosphatase, fumarylacetoacetate hydrolase, glutamate 5-kinase, glutamate-ammonia-ligase, glutaredoxin, glycerol-3-phosphate dehydrogenase, glycine cleavage system H protein, GM02LC38418-protein, heat shock protein, heat shock transcription factor, HesB/YadR/YfhF family protein, homocitrate synthase, hydrolase, isocitrate dehydrogenase, lipoprotein precursor, malate dehydrogenase, malonyl CoA-acyl carrier protein transacylase, metal-dependent hydrolase, molecular chaperone, molecular chaperone portein, monothiol glutaredoxin, multiple antibiotic resistance protein, murein transglycosylase, N-acetyl-gamma-glutamyl-phosphate reductase, NADP-dependent malic enzyme, outer-membrane lipoprotein precursor, oxidoreductase, permease protein of ABC transporter, phage-related repressor protein, phosphatase, phosphate import ATP-binding protein, phosphatidylserine synthase, phospho-2-dehydro-3-deoxyheptonate aldolase, phosphoadenosine phosphosulfate reductase, phosphotransferase, phosphotransferase system component, polygalacturonase, protein kinase, protein phosphatase, purine nucleoside phosphorylase, pyridoxamine kinase, recombinase A, sec-independent protein translocase, Sec-independent protein translocase subunit, sensor histidine kinase, sensor kinase, sll0281-protein, sll0406-protein, small nuclear ribonucleoprotein , sodium/proton antiporter, squalene monooxygenase, sugar transport system permease protein, threonine dehydratase, transcription factor, transcriptional regulator, transcriptional regulator protein, transport protein, tryptophan synthase alpha chain, TTC1386-protein, vacuolar processing enzyme, ycl027w-protein, ycl049c-protein, ydr273w-protein, ydr339c-protein, YFL019C-protein, ygl237c-protein, YGR104C-protein, yhr033w-protein, YJL127W-A-protein, ylr326w-protein, yml083c-protein, yml084w-protein, or ynl321w-protein, which respectively encode a protein comprising a polypeptide encoded by a nucleic acid sequence as shown in Table I, application no. 12, column 5 or 8, (preferably the coding region thereof), or a homolog or a fragment thereof, which respectively encode a protein comprising a polypeptide as depicted in Table II, application no. 12, column 5 or 8, or a homolg or a fragment thereof, and/or which respectively can be amplified with the primer set shown in Table III, application no. 12, column 8, are also referred to as “FCRP genes”.

Proteins or polypeptides encoded by “FCRP-genes” are referred to as “Fine Chemical Related Proteins” or “FCRP”. For the purposes of the description of the present invention, the respective protein having an activity of 2,3-dihydroxyphenylpropionate 1,2-dioxygenase, 2-C-methyl-D-erythritol 4-phosphate cytidylyltransferase, 30S ribosomal protein S3, 3-ketoacyl-CoA thiolase, 3-oxoacyl-[acyl-carrier-protein] reductase, 3-oxoacyl-[acyl-carrier-protein] synthase, 40S ribosomal protein S17, ABC transporter component, ABC transporter permease protein, acetyl-coenzyme A synthetase, acid shock protein, acyl-CoA synthase, acyldesaturase, adenosine kinase, aldolase, alkaline phosphatase isozyme conversion protein, amino acid ABC transporter permease protein, aminotransferase, ankyrin repeat protein, At2g33510-protein, ATPase epsilon subunit, ATP-binding component of a transport system, b0050-protein, b0249-protein, b0371-protein, b0518-protein, b0801-protein, b1046-protein, b1346-protein, b1470-protein, b1522-protein, b1670-protein, b1703-protein, b1837-protein, b2032-protein, b2099-protein, b2107-protein, b2126-protein, b2139-protein, b2360-protein, b2474-protein, b2513-protein, b2654-protein, b3442-protein, b3644-protein, b3812-protein, b3944-protein, b4023-protein, b4205-protein, beta-hydroxylase, betaine aldehyde dehydrogenase, bifunctional protein (phosphoribosyltransferase and regulatory protein), bifunctional purine biosynthesis protein, binding-protein-dependent transport systems inner membrane component, biotin synthase, calcium-dependent protein kinase, carbon dioxide concentrating mechanism protein, cardiolipin synthase, CDP-diacylglycerol-serine O-phosphatidyltransferase, cell division control protein, cell wall protein, chlorophyllase, citrate synthase, coproporphyrinogen III oxidase, cyclin, cystathionine gamma-synthase, cysteine synthase A, dihydrolipoamide dehydrogenase, DNA mismatch repair protein, DnaJ-like chaperone, electron transport complex protein, Elongation factor Tu, ethanolamine utilization protein, F-box protein, ferredoxin, fructose 1,6-bisphosphatase, fumarylacetoacetate hydrolase, glutamate 5-kinase, glutamate-ammonia-ligase, glutaredoxin, glycerol-3-phosphate dehydrogenase, glycine cleavage system H protein, GM02LC38418-protein, heat shock protein, heat shock transcription factor, HesB/YadR/YfhF family protein, homocitrate synthase, hydrolase, isocitrate dehydrogenase, lipoprotein precursor, malate dehydrogenase, malonyl CoA-acyl carrier protein transacylase, metal-dependent hydrolase, molecular chaperone, molecular chaperone portein, monothiol glutaredoxin, multiple antibiotic resistance protein, murein transglycosylase, N-acetyl-gamma-glutamyl-phosphate reductase, NADP-dependent malic enzyme, outer-membrane lipoprotein precursor, oxidoreductase, permease protein of ABC transporter, phage-related repressor protein, phosphatase, phosphate import ATP-binding protein, phosphatidylserine synthase, phospho-2-dehydro-3-deoxyheptonate aldolase, phosphoadenosine phosphosulfate reductase, phosphotransferase, phosphotransferase system component, polygalacturonase, protein kinase, protein phosphatase, purine nucleoside phosphorylase, pyridoxamine kinase, recombinase A, secindependent protein translocase, Sec-independent protein translocase subunit, sensor histidine kinase, sensor kinase, sll0281-protein, sll0406-protein, small nuclear ribonucleoprotein, sodium/proton antiporter, squalene monooxygenase, sugar transport system permease protein, threonine dehydratase, transcription factor, transcriptional regulator, transcriptional regulator protein, transport protein, tryptophan synthase alpha chain, TTC1386-protein, vacuolar processing enzyme, ycl027w-protein, ycl049c-protein, ydr273w-protein, ydr339c-protein, YFL019C-protein, ygl237c-protein, YGR104C-protein, yhr033w-protein, YJL127W-A-protein, ylr326w-protein, yml083c-protein, yml084w-protein, or ynl321w-protein, the respective protein comprising a polypeptide encoded by one or more respective nucleic acid sequences as shown in Table I, application no. 12, column 5 or 8, (preferably the coding region thereof), or a homolog or fragment thereof, the respective protein comprising a respective polypeptide as depicted in Table II, application no. 12, column 5 or 8, or a homolog or fragment thereof, the respective protein comprising a sequence corresponding to the consensus sequence as shown in Table IV, application no. 12, column 8, and/or the respective protein comprising at least one polypeptide motif as shown in Table IV, application no. 12, column 8 is also referred to as Fine Chemical Related Protein” or “FCRP”.

Thus, in one embodiment, the present invention provides a process of the production of 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, and/or oleic acid,respectively, by increasing or generating one or more activities, especially selected from the group consisting of 2,3-dihydroxyphenylpropionate 1,2-dioxygenase, 2-C-methyl-D-erythritol 4-phosphate cytidylyltransferase, 30S ribosomal protein S3, 3-ketoacyl-CoA thiolase, 3-oxoacyl-[acyl-carrier-protein] reductase, 3-oxoacyl-[acyl-carrier-protein] synthase, 40S ribosomal protein S17, ABC transporter component, ABC transporter permease protein, acetyl-coenzyme A synthetase, acid shock protein, acyl-CoA synthase, acyl-desaturase, adenosine kinase, aldolase, alkaline phosphatase isozyme conversion protein, amino acid ABC transporter permease protein, aminotransferase, ankyrin repeat protein, At2g33510-protein, ATPase epsilon subunit, ATP-binding component of a transport system, b0050-protein, b0249-protein, b0371-protein, b0518-protein, b0801-protein, b1046-protein, b1346-protein, b1470-protein, b1522-protein, b1670-protein, b1703-protein, b1837-protein, b2032-protein, b2099-protein, b2107-protein, b2126-protein, b2139-protein, b2360-protein, b2474-protein, b2513-protein, b2654-protein, b3442-protein, b3644-protein, b3812-protein, b3944-protein, b4023-protein, b4205-protein, beta-hydroxylase, betaine aldehyde dehydrogenase, bifunctional protein (phosphoribosyltransferase and regulatory protein), bifunctional purine biosynthesis protein, binding-protein-dependent transport systems inner membrane component, biotin synthase, calcium-dependent protein kinase, carbon dioxide concentrating mechanism protein, cardiolipin synthase, CDP-diacylglycerol-serine O-phosphatidyltransferase, cell division control protein, cell wall protein, chlorophyllase, citrate synthase, coproporphyrinogen III oxidase, cyclin, cystathionine gamma-synthase, cysteine synthase A, dihydrolipoamide dehydrogenase, DNA mismatch repair protein, DnaJ-like chaperone, electron transport complex protein, Elongation factor Tu, ethanolamine utilization protein, F-box protein, ferredoxin, fructose 1,6-bisphosphatase, fumarylacetoacetate hydrolase, glutamate 5-kinase, glutamate-ammonia-ligase, glutaredoxin, glycerol-3-phosphate dehydrogenase, glycine cleavage system H protein, GM02LC38418-protein, heat shock protein, heat shock transcription factor, HesB/YadR/YfhF family protein, homocitrate synthase, hydrolase, isocitrate dehydrogenase, lipoprotein precursor, malate dehydrogenase, malonyl CoA-acyl carrier protein transacylase, metal-dependent hydrolase, molecular chaperone, molecular chaperone portein, monothiol glutaredoxin, multiple antibiotic resistance protein, murein transglycosylase, N-acetyl-gamma-glutamyl-phosphate reductase, NADP-dependent malic enzyme, outer-membrane lipoprotein precursor, oxidoreductase, permease protein of ABC transporter, phage-related repressor protein, phosphatase, phosphate import ATP-binding protein, phosphatidylserine synthase, phospho-2-dehydro-3-deoxyheptonate aldolase, phosphoadenosine phosphosulfate reductase, phosphotransferase, phosphotransferase system component, polygalacturonase, protein kinase, protein phosphatase, purine nucleoside phosphorylase, pyridoxamine kinase, recombinase A, sec-independent protein translocase, Sec-independent protein translocase subunit, sensor histidine kinase, sensor kinase, sll0281-protein, sll0406-protein, small nuclear ribonucleoprotein , sodium/proton antiporter, squalene monooxygenase, sugar transport system permease protein, threonine dehydratase, transcription factor, transcriptional regulator, transcriptional regulator protein, transport protein, tryptophan synthase alpha chain, TTC1386-protein, vacuolar processing enzyme, ycl027w-protein, ycl049c-protein, ydr273w-protein, ydr339c-protein, YFL019C-protein, ygl237c-protein, YGR104C-protein, yhr033w-protein, YJL127W-A-protein, ylr326w-protein, yml083c-protein, yml084w-protein, and ynl321w-protein, which is conferred by one or more FCRPs or the gene product of one or more FCRP-genes, for example by the gene product of a nucleic acid sequences comprising a polynucleotide selected from the group as shown in Table I, application no. 12, column 5 or 8, (preferably by the coding region thereof), or a homolog or a fragment thereof, e.g. or by one or more proteins each comprising a polypeptide encoded by one or more nucleic acid sequences selected from the group as shown in Table I, application no. 12, column 5 or 8, (preferably by the coding region thereof), or a homolog or a fragment thereof, or by one or more protein(s) each comprising a polypeptide selected from the group as depicted in Table II, application no. 12, column 5 and 8, or a homolog thereof, or a protein comprising a sequence corresponding to the consensus sequence or comprising at least one polypeptide motif as shown in Table IV, application no. 12, column 8.

for the disclosure of this paragraph see [0025.1.8.8] above.

In an embodiment, the process comprises increasing or generating the activity of one or more polypeptides having said activity, e.g. by generating or increasing the amount and/or specific activity in the cell or a compartment of a cell of one of more FCRP, especially selected from the group consisting of 2,3-dihydroxyphenylpropionate 1,2-dioxygenase, 2-C-methyl-D-erythritol 4-phosphate cytidylyltransferase, 30S ribosomal protein S3, 3-ketoacyl-CoA thiolase, 3-oxoacyl-[acyl-carrier-protein] reductase, 3-oxoacyl-[acyl-carrier-protein] synthase, 40S ribosomal protein S17, ABC transporter component, ABC transporter permease protein, acetyl-coenzyme A synthetase, acid shock protein, acyl-CoA synthase, acyl-desaturase, adenosine kinase, aldolase, alkaline phosphatase isozyme conversion protein, amino acid ABC transporter permease protein, aminotransferase, ankyrin repeat protein, At2g33510-protein, ATPase epsilon subunit, ATP-binding component of a transport system, b0050-protein, b0249-protein, b0371-protein, b0518-protein, b0801-protein, b1046-protein, b1346-protein, b1470-protein, b1522-protein, b1670-protein, b1703-protein, b1837-protein, b2032-protein, b2099-protein, b2107-protein, b2126-protein, b2139-protein, b2360-protein, b2474-protein, b2513-protein, b2654-protein, b3442-protein, b3644-protein, b3812-protein, b3944-protein, b4023-protein, b4205-protein, beta-hydroxylase, betaine aldehyde dehydrogenase, bifunctional protein (phosphoribosyltransferase and regulatory protein), bifunctional purine biosynthesis protein, binding-protein-dependent transport systems inner membrane component, biotin synthase, calcium-dependent protein kinase, carbon dioxide concentrating mechanism protein, cardiolipin synthase, CDP-diacylglycerol-serine O-phosphatidyltransferase, cell division control protein, cell wall protein, chlorophyllase, citrate synthase, coproporphyrinogen III oxidase, cyclin, cystathionine gamma-synthase, cysteine synthase A, dihydrolipoamide dehydrogenase, DNA mismatch repair protein, DnaJ-like chaperone, electron transport complex protein, Elongation factor Tu, ethanolamine utilization protein, F-box protein, ferredoxin, fructose 1,6-bisphosphatase, fumarylacetoacetate hydrolase, glutamate 5-kinase, glutamate-ammonia-ligase, glutaredoxin, glycerol-3-phosphate dehydrogenase, glycine cleavage system H protein, GM02LC38418-protein, heat shock protein, heat shock transcription factor, HesB/YadR/YfhF family protein, homocitrate synthase, hydrolase, isocitrate dehydrogenase, lipoprotein precursor, malate dehydrogenase, malonyl CoA-acyl carrier protein transacylase, metal-dependent hydrolase, molecular chaperone, molecular chaperone portein, monothiol glutaredoxin, multiple antibiotic resistance protein, murein transglycosylase, N-acetyl-gamma-glutamyl-phosphate reductase, NADP-dependent malic enzyme, outer-membrane lipoprotein precursor, oxidoreductase, permease protein of ABC transporter, phage-related repressor protein, phosphatase, phosphate import ATP-binding protein, phosphatidylserine synthase, phospho-2-dehydro-3-deoxyheptonate aldolase, phosphoadenosine phosphosulfate reductase, phosphotransferase, phosphotransferase system component, polygalacturonase, protein kinase, protein phosphatase, purine nucleoside phosphorylase, pyridoxamine kinase, recombinase A, sec-independent protein translocase, Sec-independent protein translocase subunit, sensor histidine kinase, sensor kinase, sll0281-protein, sll0406-protein, small nuclear ribonucleoprotein, sodium/proton antiporter, squalene monooxygenase, sugar transport system permease protein, threonine dehydratase, transcription factor, transcriptional regulator, transcriptional regulator protein, transport protein, tryptophan synthase alpha chain, TTC1386-protein, vacuolar processing enzyme, ycl027w-protein, ycl049c-protein, ydr273w-protein, ydr339c-protein, YFL019C-protein, yg1237c-protein, YGR104C-protein, yhr033w-protein, YJL127W-A-protein, ylr326w-protein, yml083c-protein, yml084w-protein, and ynl321w-protein, for example of the respective polypeptide as depicted in Table II, application no. 12, column 5 and 8, or a homolog or a fragment thereof, or the respective polypeptide comprising a sequence corresponding to the consensus sequences as shown in Table IV, application no. 12, column 8, or the respective polypeptide comprising at least one polypeptide motif as depicted in Table IV, application no. 12, column 8.

A further embodiment of the present invention relates to a process for the production of cerebronic acid, which comprises
(a) increasing or generating the activity of a chlorophyllase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a chlorophyllase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of hexadecatrienoic acid, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of cerebronic acid, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of hexadecatrienoic acid, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxybehenic acid, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxypalmitic acid, which comprises
(a) increasing or generating the activity of a At2g33510-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of cerebronic acid, which comprises
(a) increasing or generating the activity of a adenosine kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of 2-hydroxybehenic acid, which comprises
(a) increasing or generating the activity of a adenosine kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a DNA mismatch repair protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of cerebronic acid, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of hexadecatrienoic acid, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxybehenic acid, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxypalmitic acid, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of 2-hydroxybehenic acid, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxypalmitic acid, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of hexadecatrienoic acid, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxybehenic acid, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of cerebronic acid, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of 2-hydroxybehenic acid, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human

A furthogenribardiment of the present invention relates to a process for the production of cerebronic acid, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of hexadecadienoic acid, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecatrienoic acid, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxybehenic acid, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxypalmitic acid, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of oleic acid, which comprises
(a) increasing or generating the activity of a acyl-desaturase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

A further embodiment of the present invention relates to a process for the production of hexadecatrienoic acid, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of hexadecatrienoic acid, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of hexadecadienoic acid, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of cerebronic acid, which comprises
(a) increasing or generating the activity of a CDP-diacylglycerol-serine O-phosphatidyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of 2-hydroxybehenic acid, which comprises
(a) increasing or generating the activity of a CDP-diacylglycerol-serine O-phosphatidyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a CDP-diacylglycerol-serine O-phosphatidyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxypalmitic acid, which comprises
(a) increasing or generating the activity of a CDP-diacylglycerol-serine O-phosphatidyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of hexadecadienoic acid, which comprises
(a) increasing or generating the activity of a sec-independent protein translocase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a transcriptional regulator protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxypalmitic acid, which comprises
(a) increasing or generating the activity of a beta-hydroxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of hexadecadienoic acid, which comprises
(a) increasing or generating the activity of a 30S ribosomal protein S3 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of 2-hydroxybehenic acid, which comprises
(a) increasing or generating the activity of a acyl-CoA synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a heat shock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of hexadecadienoic acid, which comprises
(a) increasing or generating the activity of a recombinase A non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of lauric acid, which comprises
(a) increasing or generating the activity of a ABC transporter component non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lauric acid or a composition comprising lauric acid in said non-human organism or in the culture medium surrounding said non-human organism.

A further embodiment of the present invention relates to a process for the production of 2-hydroxypalmitic acid, which comprises
(a) increasing or generating the activity of a hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of 2-hydroxypalmitic acid, which comprises
(a) increasing or generating the activity of a glutamate-ammonia-ligase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of 2-hydroxybehenic acid, which comprises
(a) increasing or generating the activity of a fumarylacetoacetate hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a N-acetyl-gamma-glutamyl-phosphate reductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a HesB/YadR/YfhF family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a purine nucleoside phosphorylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxybehenic acid, which comprises
(a) increasing or generating the activity of a b0050-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a b0050-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of cerebronic acid, which comprises
(a) increasing or generating the activity of a b0249-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of behenic acid, which comprises
(a) increasing or generating the activity of a betaine aldehyde dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of behenic acid or a composition comprising behenic acid in said non-human organism or in the culture medium surrounding said non-human organism.

A further embodiment of the present invention relates to a process for the production of eicosanoic acid, which comprises
(a) increasing or generating the activity of a betaine aldehyde dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of eicosanoic acid or a composition comprising eicosanoic acid in said non-human organism or in the culture medium surrounding said non-human organism.

A further embodiment of the present invention relates to a process for the production of myristic acid, which comprises
(a) increasing or generating the activity of a 2,3-dihydroxyphenylpropionate 1,2-dioxygenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myristic acid or a composition comprising myristic acid in said non-human organism or in the culture medium surrounding said non-human organism.

A further embodiment of the present invention relates to a process for the production of oleic acid, which comprises
(a) increasing or generating the activity of a 2,3-dihydroxyphenylpropionate 1,2-dioxygenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

A further embodiment of the present invention relates to a process for the production of hexadecatrienoic acid, which comprises
(a) increasing or generating the activity of a b0371-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of cerebronic acid, which comprises
(a) increasing or generating the activity of a ATP-binding component of a transport system non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of 2-hydroxybehenic acid, which comprises
(a) increasing or generating the activity of a ATP-binding component of a transport system non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxypalmitic acid, which comprises
(a) increasing or generating the activity of a ATP-binding component of a transport system non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of hexadecadienoic acid, which comprises
(a) increasing or generating the activity of a b0518-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of 2-hydroxybehenic acid, which comprises
(a) increasing or generating the activity of a sensor histidine kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a sensor histidine kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of cerebronic acid, which comprises
(a) increasing or generating the activity of a citrate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a b0801-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of cerebronic acid, which comprises
(a) increasing or generating the activity of a b1046-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of hexadecatrienoic acid, which comprises
(a) increasing or generating the activity of a malonyl CoA-acyl carrier protein transacylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of hexadecadienoic acid, which comprises
(a) increasing or generating the activity of a 3-oxoacyl-[acyl-carrier-protein] reductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecatrienoic acid, which comprises
(a) increasing or generating the activity of a 3-oxoacyl-[acyl-carrier-protein] reductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of hexadecatrienoic acid, which comprises
(a) increasing or generating the activity of a 3-oxoacyl-[acyl-carrier-protein] synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxybehenic acid, which comprises
(a) increasing or generating the activity of a 3-oxoacyl-[acyl-carrier-protein] synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of oleic acid, which comprises
(a) increasing or generating the activity of a sodium/proton antiporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

A further embodiment of the present invention relates to a process for the production of 2-hydroxypalmitic acid, which comprises
(a) increasing or generating the activity of a sodium/proton antiporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of behenic acid, which comprises
(a) increasing or generating the activity of a outer-membrane lipoprotein precursor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of behenic acid or a composition comprising behenic acid in said non-human organism or in the culture medium surrounding said non-human organism.

A further embodiment of the present invention relates to a process for the production of behenic acid, which comprises
(a) increasing or generating the activity of a cardiolipin synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of behenic acid or a composition comprising behenic acid in said non-human organism or in the culture medium surrounding said non-human organism.

A further embodiment of the present invention relates to a process for the production of heptadecanoic acid, which comprises
(a) increasing or generating the activity of a cardiolipin synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of heptadecanoic acid or a composition comprising heptadecanoic acid in said non-human organism or in the culture medium surrounding said non-human organism.

A further embodiment of the present invention relates to a process for the production of hexadecatrienoic acid, which comprises
(a) increasing or generating the activity of a b1346-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of lauric acid, which comprises
(a) increasing or generating the activity of a phage-related repressor protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lauric acid or a composition comprising lauric acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a lipoprotein precursor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of hexadecatrienoic acid, which comprises
(a) increasing or generating the activity of a b1470-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of hexadecatrienoic acid, which comprises
(a) increasing or generating the activity of a NADP-dependent malic enzyme non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxybehenic acid, which comprises
(a) increasing or generating the activity of a b1522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of hexadecatrienoic acid, which comprises
(a) increasing or generating the activity of a acid shock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a phosphotransferase system component non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxybehenic acid, which comprises
(a) increasing or generating the activity of a electron transport complex protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of hexadecatrienoic acid, which comprises
(a) increasing or generating the activity of a pyridoxamine kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a b1670-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of hexadecadienoic acid, which comprises
(a) increasing or generating the activity of a b1703-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecatrienoic acid, which comprises
(a) increasing or generating the activity of a b1703-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of hexadecadienoic acid, which comprises
(a) increasing or generating the activity of a transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecadienoic acid, which comprises
(a) increasing or generating the activity of a b1837-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecatrienoic acid, which comprises
(a) increasing or generating the activity of a b1837-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of hexadecadienoic acid, which comprises
(a) increasing or generating the activity of a b2032-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecadienoic acid, which comprises
(a) increasing or generating the activity of a b2099-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecatrienoic acid, which comprises
(a) increasing or generating the activity of a b2099-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a b2107-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of lauric acid, which comprises
(a) increasing or generating the activity of a b2126-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lauric acid or a composition comprising lauric acid in said non-human organism or in the culture medium surrounding said non-human organism.

A further embodiment of the present invention relates to a process for the production of 2-hydroxypalmitic acid, which comprises
(a) increasing or generating the activity of a b2139-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of hexadecatrienoic acid, which comprises
(a) increasing or generating the activity of a ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxybehenic acid, which comprises
(a) increasing or generating the activity of a sensor kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a b2360-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a cysteine synthase A non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of hexadecadienoic acid, which comprises
(a) increasing or generating the activity of a ethanolamine utilization protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecatrienoic acid, which comprises
(a) increasing or generating the activity of a ethanolamine utilization protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of oleic acid, which comprises
(a) increasing or generating the activity of a ethanolamine utilization protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

A further embodiment of the present invention relates to a process for the production of cerebronic acid, which comprises
(a) increasing or generating the activity of a b2474-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of hexadecatrienoic acid, which comprises
(a) increasing or generating the activity of a b2474-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a b2474-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of hexadecadienoic acid, which comprises
(a) increasing or generating the activity of a b2513-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecadienoic acid, which comprises
(a) increasing or generating the activity of a phosphatidylserine synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a b2654-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of hexadecatrienoic acid, which comprises
(a) increasing or generating the activity of a 2-C-methyl-D-erythritol 4-phosphate cytidylyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of eicosanoic acid, which comprises
(a) increasing or generating the activity of a alkaline phosphatase isozyme conversion protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of eicosanoic acid or a composition comprising eicosanoic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a murein transglycosylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of hexadecadienoic acid, which comprises
(a) increasing or generating the activity of a b3442-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of cerebronic acid, which comprises
(a) increasing or generating the activity of a sugar transport system permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of 2-hydroxybehenic acid, which comprises
(a) increasing or generating the activity of a b3812-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of hexadecadienoic acid, which comprises
(a) increasing or generating the activity of a 3-ketoacyl-CoA thiolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of oleic acid, which comprises
(a) increasing or generating the activity of a 3-ketoacyl-CoA thiolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

A further embodiment of the present invention relates to a process for the production of hexadecadienoic acid, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecadienoic acid, which comprises
(a) increasing or generating the activity of a fructose 1,6-bisphosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of behenic acid, which comprises
(a) increasing or generating the activity of a cystathionine gamma-synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of behenic acid or a composition comprising behenic acid in said non-human organism or in the culture medium surrounding said non-human organism.

A further embodiment of the present invention relates to a process for the production of heptadecanoic acid, which comprises
(a) increasing or generating the activity of a cystathionine gamma-synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of heptadecanoic acid or a composition comprising heptadecanoic acid in said non-human organism or in the culture medium surrounding said non-human organism.

A further embodiment of the present invention relates to a process for the production of oleic acid, which comprises
(a) increasing or generating the activity of a cystathionine gamma-synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

A further embodiment of the present invention relates to a process for the production of 2-hydroxypalmitic acid, which comprises
(a) increasing or generating the activity of a b3944-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of 2-hydroxybehenic acid, which comprises
(a) increasing or generating the activity of a ankyrin repeat protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of hexadecadienoic acid, which comprises
(a) increasing or generating the activity of a b4023-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of heptadecanoic acid, which comprises
(a) increasing or generating the activity of a acetyl-coenzyme A synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of heptadecanoic acid or a composition comprising heptadecanoic acid in said non-human organism or in the culture medium surrounding said non-human organism.

A further embodiment of the present invention relates to a process for the production of 2-hydroxypalmitic acid, which comprises
(a) increasing or generating the activity of a b4205-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of 2-hydroxybehenic acid, which comprises
(a) increasing or generating the activity of a vacuolar processing enzyme non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxypalmitic acid, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of cerebronic acid, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of hexadecatrienoic acid, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a GM02LC38418-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of hexadecadienoic acid, which comprises
(a) increasing or generating the activity of a sll0281-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a bifunctional protein (phosphoribosyltransferase and regulatory protein) non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a sll0406-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of cerebronic acid, which comprises
(a) increasing or generating the activity of a binding-protein-dependent transport systems inner membrane component non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of cerebronic acid, which comprises
(a) increasing or generating the activity of a phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of hexadecadienoic acid, which comprises
(a) increasing or generating the activity of a carbon dioxide concentrating mechanism protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecatrienoic acid, which comprises
(a) increasing or generating the activity of a carbon dioxide concentrating mechanism protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of cerebronic acid, which comprises
(a) increasing or generating the activity of a coproporphyrinogen III oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a coproporphyrinogen III oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxypalmitic acid, which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of hexadecatrienoic acid, which comprises
(a) increasing or generating the activity of a bifunctional purine biosynthesis protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of cerebronic acid, which comprises
(a) increasing or generating the activity of a malate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of hexadecatrienoic acid, which comprises
(a) increasing or generating the activity of a glycine cleavage system H protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a permease protein of ABC transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of hexadecadienoic acid, which comprises
(a) increasing or generating the activity of a tryptophan synthase alpha chain non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of 2-hydroxybehenic acid, which comprises
(a) increasing or generating the activity of a dihydrolipoamide dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxypalmitic acid, which comprises
(a) increasing or generating the activity of a phosphate import ATP-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of hexadecatrienoic acid, which comprises
(a) increasing or generating the activity of a glutamate 5-kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of cerebronic acid, which comprises
(a) increasing or generating the activity of a threonine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of hexadecatrienoic acid, which comprises
(a) increasing or generating the activity of a threonine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a threonine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a ferredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxybehenic acid, which comprises
(a) increasing or generating the activity of a Sec-independent protein translocase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a amino acid ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of cerebronic acid, which comprises
(a) increasing or generating the activity of a metal-dependent hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of hexadecadienoic acid, which comprises
(a) increasing or generating the activity of a metal-dependent hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of 2-hydroxybehenic acid, which comprises
(a) increasing or generating the activity of a multiple antibiotic resistance protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of hexadecatrienoic acid, which comprises
(a) increasing or generating the activity of a TTC1386-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxybehenic acid, which comprises
(a) increasing or generating the activity of a homocitrate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of hexadecadienoic acid, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecatrienoic acid, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of hexadecadienoic acid, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecatrienoic acid, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of hexadecatrienoic acid, which comprises
(a) increasing or generating the activity of a cell division control protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of cerebronic acid, which comprises
(a) increasing or generating the activity of a ycl027w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a ycl027w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxypalmitic acid, which comprises
(a) increasing or generating the activity of a ycl049c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of oleic acid, which comprises
(a) increasing or generating the activity of a glycerol-3-phosphate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

A further embodiment of the present invention relates to a process for the production of eicosanoic acid, which comprises
(a) increasing or generating the activity of a isocitrate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of eicosanoic acid or a composition comprising eicosanoic acid in said non-human organism or in the culture medium surrounding said non-human organism.

A further embodiment of the present invention relates to a process for the production of heptadecanoic acid, which comprises
(a) increasing or generating the activity of a isocitrate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of heptadecanoic acid or a composition comprising heptadecanoic acid in said non-human organism or in the culture medium surrounding said non-human organism.

A further embodiment of the present invention relates to a process for the production of hexadecadienoic acid, which comprises
(a) increasing or generating the activity of a cell division control protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecatrienoic acid, which comprises
(a) increasing or generating the activity of a cell division control protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxybehenic acid, which comprises
(a) increasing or generating the activity of a homocitrate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of behenic acid, which comprises
(a) increasing or generating the activity of a phospho-2-dehydro-3-deoxyheptonate aldolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of behenic acid or a composition comprising behenic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a cell wall protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a F-box protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of cerebronic acid, which comprises
(a) increasing or generating the activity of a ydr273w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a ydr273w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of hexadecatrienoic acid, which comprises
(a) increasing or generating the activity of a ydr339c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of heptadecanoic acid, which comprises
(a) increasing or generating the activity of a aldolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of heptadecanoic acid or a composition comprising heptadecanoic acid in said non-human organism or in the culture medium surrounding said non-human organism.

A further embodiment of the present invention relates to a process for the production of myristic acid, which comprises
(a) increasing or generating the activity of a small nuclear ribonucleoprotein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myristic acid or a composition comprising myristic acid in said non-human organism or in the culture medium surrounding said non-human organism.

A further embodiment of the present invention relates to a process for the production of hexadecadienoic acid, which comprises
(a) increasing or generating the activity of a molecular chaperone portein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecatrienoic acid, which comprises
(a) increasing or generating the activity of a molecular chaperone portein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxybehenic acid, which comprises
(a) increasing or generating the activity of a YFL019C-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxypalmitic acid, which comprises
(a) increasing or generating the activity of a YFL019C-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of hexadecadienoic acid, which comprises
(a) increasing or generating the activity of a aminotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecatrienoic acid, which comprises
(a) increasing or generating the activity of a ygl237c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of eicosanoic acid, which comprises
(a) increasing or generating the activity of a YGR104C-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of eicosanoic acid or a composition comprising eicosanoic acid in said non-human organism or in the culture medium surrounding said non-human organism.

A further embodiment of the present invention relates to a process for the production of cerebronic acid, which comprises
(a) increasing or generating the activity of a squalene monooxygenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of hexadecadienoic acid, which comprises
(a) increasing or generating the activity of a biotin synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of cerebronic acid, which comprises
(a) increasing or generating the activity of a yhr033w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of cerebronic acid, which comprises
(a) increasing or generating the activity of a DnaJ-like chaperone non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of 2-hydroxybehenic acid, which comprises
(a) increasing or generating the activity of a YJL127W-A-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a cystathionine gamma-synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxypalmitic acid, which comprises
(a) increasing or generating the activity of a cystathionine gamma-synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of 2-hydroxypalmitic acid, which comprises
(a) increasing or generating the activity of a polygalacturonase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of hexadecatrienoic acid, which comprises
(a) increasing or generating the activity of a cyclin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of hexadecadienoic acid, which comprises
(a) increasing or generating the activity of a ylr326w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecatrienoic acid, which comprises
(a) increasing or generating the activity of a ylr326w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxybehenic acid, which comprises
(a) increasing or generating the activity of a yml083c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a yml084w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxypalmitic acid, which comprises
(a) increasing or generating the activity of a molecular chaperone non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of oleic acid, which comprises
(a) increasing or generating the activity of a phosphotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

A further embodiment of the present invention relates to a process for the production of 2-hydroxybehenic acid, which comprises
(a) increasing or generating the activity of a ynl321w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of hexadecatrienoic acid, which comprises
(a) increasing or generating the activity of a Elongation factor Tu non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a ATPase epsilon subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxypalmitic acid, which comprises
(a) increasing or generating the activity of a phosphoadenosine phosphosulfate reductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of oleic acid, which comprises
(a) increasing or generating the activity of a 40S ribosomal protein S17 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

A further embodiment of the present invention relates to a process for the production of 2-hydroxybehenic acid, which comprises
(a) increasing or generating the activity of a b3644-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of behenic acid, which comprises
(a) increasing or generating the activity of a b3644-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of behenic acid or a composition comprising behenic acid in said non-human organism or in the culture medium surrounding said non-human organism.

A further embodiment of the present invention relates to a process for the production of nervonic acid, which comprises
(a) increasing or generating the activity of a b3644-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of nervonic acid or a composition comprising nervonic acid in said non-human organism or in the culture medium surrounding said non-human organism.

A further embodiment of the present invention relates to a process for the production of 2-hydroxybehenic acid, which comprises
(a) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical 2-hydroxybehenic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical 2-hydroxybehenic acid;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of 2-hydroxybehenic acid, or a composition comprising 2-hydroxybehenic acid in said non-human organism or in the culture medium surrounding said non-human organism.

A further embodiment of the present invention relates to a process for the production of 2-hydroxypalmitic acid, which comprises
(a) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical 2-hydroxypalmitic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical 2-hydroxypalmitic acid;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of 2-hydroxypalmitic acid, or a composition comprising 2-hydroxypalmitic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical 2-hydroxytetracosenoic acid (preferably 2-hydroxynervonic acid); or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical 2-hydroxytetracosenoic acid (preferably 2-hydroxynervonic acid);
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of 2-hydroxytetracosenoic acid (preferably 2-hydroxynervonic acid), or a composition comprising 2-hydroxytetracosenoic acid (preferably 2-hydroxynervonic acid) in said non-human organism or in the culture medium surrounding said non-human organism.

A further embodiment of the present invention relates to a process for the production of behenic acid, which comprises
(a) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical behenic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical behenic acid;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of behenic acid, or a composition comprising behenic acid in said non-human organism or in the culture medium surrounding said non-human organism.

A further embodiment of the present invention relates to a process for the production of cerebronic acid, which comprises
(a) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical cerebronic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical cerebronic acid;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of cerebronic acid, or a composition comprising cerebronic acid in said non-human organism or in the culture medium surrounding said non-human organism.

A further embodiment of the present invention relates to a process for the production of eicosanoic acid, which comprises
(a) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical eicosanoic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical eicosanoic acid;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of eicosanoic acid, or a composition comprising eicosanoic acid in said non-human organism or in the culture medium surrounding said non-human organism.

A further embodiment of the present invention relates to a process for the production of heptadecanoic acid, which comprises
(a) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical heptadecanoic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical heptadecanoic acid;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of heptadecanoic acid, or a composition comprising heptadecanoic acid in said non-human organism or in the culture medium surrounding said non-human organism.

A further embodiment of the present invention relates to a process for the production of hexadecadienoic acid, which comprises
(a) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical hexadecadienoic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical hexadecadienoic acid;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of hexadecadienoic acid, or a composition comprising hexadecadienoic acid in said non-human organism or in the culture medium surrounding said non-human organism.

A further embodiment of the present invention relates to a process for the production of hexadecatrienoic acid, which comprises
(a) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical hexadecatrienoic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical hexadecatrienoic acid;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of hexadecatrienoic acid, or a composition comprising hexadecatrienoic acid in said non-human organism or in the culture medium surrounding said non-human organism.

A further embodiment of the present invention relates to a process for the production of lauric acid, which comprises
(a) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical lauric acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical lauric acid;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of lauric acid, or a composition comprising lauric acid in said non-human organism or in the culture medium surrounding said non-human organism.

A further embodiment of the present invention relates to a process for the production of myristic acid, which comprises
(a) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical myristic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical myristic acid;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of myristic acid, or a composition comprising myristic acid in said non-human organism or in the culture medium surrounding said non-human organism.

A further embodiment of the present invention relates to a process for the production of nervonic acid, which comprises
(a) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical nervonic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical nervonic acid;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of nervonic acid, or a composition comprising nervonic acid in said non-human organism or in the culture medium surrounding said non-human organism.

A further embodiment of the present invention relates to a process for the production of oleic acid, which comprises
(a) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical oleic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical oleic acid;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of oleic acid, or a composition comprising oleic acid in said non-human organism or in the culture medium surrounding said non-human organism.

A further embodiment of the present invention relates to a process for the production of cerebronic acid, which comprises
(a) increasing or generating one or more activities selected from the group consisting of chlorophyllase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of chlorophyllase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of hexadecatrienoic acid, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of cerebronic acid, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of hexadecatrienoic acid, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxybehenic acid, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxypalmitic acid, which comprises
(a) increasing or generating one or more activities selected from the group consisting of At2g33510-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of cerebronic acid, which comprises
(a) increasing or generating one or more activities selected from the group consisting of adenosine kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of 2-hydroxybehenic acid, which comprises
(a) increasing or generating one or more activities selected from the group consisting of adenosine kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of DNA mismatch repair protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of cerebronic acid, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of hexadecatrienoic acid, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxybehenic acid, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxypalmitic acid, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of 2-hydroxybehenic acid, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxypalmitic acid, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of hexadecatrienoic acid, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxybehenic acid, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of cerebronic acid, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of 2-hydroxybehenic acid, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of cerebronic acid, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of hexadecadienoic acid, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecatrienoic acid, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxybehenic acid, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxypalmitic acid, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of oleic acid, which comprises
(a) increasing or generating one or more activities selected from the group consisting of acyldesaturase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

A further embodiment of the present invention relates to a process for the production of hexadecatrienoic acid, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of hexadecatrienoic acid, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of hexadecadienoic acid, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of cerebronic acid, which comprises
(a) increasing or generating one or more activities selected from the group consisting of CDP-diacylglycerol-serine O-phosphatidyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of 2-hydroxybehenic acid, which comprises
(a) increasing or generating one or more activities selected from the group consisting of CDP-diacylglycerol-serine O-phosphatidyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of CDP-diacylglycerol-serine O-phosphatidyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxypalmitic acid, which comprises
(a) increasing or generating one or more activities selected from the group consisting of CDP-diacylglycerol-serine O-phosphatidyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of hexadecadienoic acid, which comprises
(a) increasing or generating one or more activities selected from the group consisting of secindependent protein translocase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of transcriptional regulator protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxypalmitic acid, which comprises
(a) increasing or generating one or more activities selected from the group consisting of betahydroxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of hexadecadienoic acid, which comprises
(a) increasing or generating one or more activities selected from the group consisting of 30S ribosomal protein S3 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of 2-hydroxybehenic acid, which comprises
(a) increasing or generating one or more activities selected from the group consisting of acylCoA synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of heat shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of hexadecadienoic acid, which comprises
(a) increasing or generating one or more activities selected from the group consisting of recombinase A in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of lauric acid, which comprises
(a) increasing or generating one or more activities selected from the group consisting of ABC transporter component in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lauric acid or a composition comprising lauric acid in said non-human organism or in the culture medium surrounding said non-human organism.

A further embodiment of the present invention relates to a process for the production of 2-hydroxypalmitic acid, which comprises
(a) increasing or generating one or more activities selected from the group consisting of hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of 2-hydroxypalmitic acid, which comprises
(a) increasing or generating one or more activities selected from the group consisting of glutamate-ammonia-ligase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of 2-hydroxybehenic acid, which comprises
(a) increasing or generating one or more activities selected from the group consisting of fumarylacetoacetate hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of N-acetyl-gamma-glutamyl-phosphate reductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of HesB/YadR/YfhF family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of purine nucleoside phosphorylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxybehenic acid, which comprises
(a) increasing or generating one or more activities selected from the group consisting of b0050-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of b0050-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of cerebronic acid, which comprises
(a) increasing or generating one or more activities selected from the group consisting of b0249-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of behenic acid, which comprises
(a) increasing or generating one or more activities selected from the group consisting of betaine aldehyde dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of behenic acid or a composition comprising behenic acid in said non-human organism or in the culture medium surrounding said non-human organism.

A further embodiment of the present invention relates to a process for the production of eicosanoic acid, which comprises
(a) increasing or generating one or more activities selected from the group consisting of betaine aldehyde dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of eicosanoic acid or a composition comprising eicosanoic acid in said non-human organism or in the culture medium surrounding said non-human organism.

A further embodiment of the present invention relates to a process for the production of myristic acid, which comprises
(a) increasing or generating one or more activities selected from the group consisting of 2,3-dihydroxyphenylpropionate 1,2-dioxygenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myristic acid or a composition comprising myristic acid in said non-human organism or in the culture medium surrounding said non-human organism.

A further embodiment of the present invention relates to a process for the production of oleic acid, which comprises
(a) increasing or generating one or more activities selected from the group consisting of 2,3-dihydroxyphenylpropionate 1,2-dioxygenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

A further embodiment of the present invention relates to a process for the production of hexadecatrienoic acid, which comprises
(a) increasing or generating one or more activities selected from the group consisting of b0371-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of cerebronic acid, which comprises
(a) increasing or generating one or more activities selected from the group consisting of ATP-binding component of a transport system in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of 2-hydroxybehenic acid, which comprises
(a) increasing or generating one or more activities selected from the group consisting of ATP-binding component of a transport system in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxypalmitic acid, which comprises
(a) increasing or generating one or more activities selected from the group consisting of ATP-binding component of a transport system in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of hexadecadienoic acid, which comprises
(a) increasing or generating one or more activities selected from the group consisting of b0518-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of 2-hydroxybehenic acid, which comprises
(a) increasing or generating one or more activities selected from the group consisting of sensor histidine kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of sensor histidine kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of cerebronic acid, which comprises
(a) increasing or generating one or more activities selected from the group consisting of citrate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of b0801-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of cerebronic acid, which comprises
(a) increasing or generating one or more activities selected from the group consisting of b1046-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of hexadecatrienoic acid, which comprises
(a) increasing or generating one or more activities selected from the group consisting of malonyl CoA-acyl carrier protein transacylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of hexadecadienoic acid, which comprises
(a) increasing or generating one or more activities selected from the group consisting of 3-oxoacyl-[acyl-carrier-protein] reductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecatrienoic acid, which comprises
(a) increasing or generating one or more activities selected from the group consisting of 3-oxoacyl-[acyl-carrier-protein] reductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of hexadecatrienoic acid, which comprises
(a) increasing or generating one or more activities selected from the group consisting of 3-oxoacyl-[acyl-carrier-protein] synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxybehenic acid, which comprises
(a) increasing or generating one or more activities selected from the group consisting of 3-oxoacyl-[acyl-carrier-protein] synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of oleic acid, which comprises
(a) increasing or generating one or more activities selected from the group consisting of sodium/proton antiporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

A further embodiment of the present invention relates to a process for the production of 2-hydroxypalmitic acid, which comprises
(a) increasing or generating one or more activities selected from the group consisting of sodium/proton antiporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of behenic acid, which comprises
(a) increasing or generating one or more activities selected from the group consisting of outer-membrane lipoprotein precursor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of behenic acid or a composition comprising behenic acid in said non-human organism or in the culture medium surrounding said non-human organism.

A further embodiment of the present invention relates to a process for the production of behenic acid, which comprises
(a) increasing or generating one or more activities selected from the group consisting of cardiolipin synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of behenic acid or a composition comprising behenic acid in said non-human organism or in the culture medium surrounding said non-human organism.

A further embodiment of the present invention relates to a process for the production of heptadecanoic acid, which comprises
(a) increasing or generating one or more activities selected from the group consisting of cardiolipin synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of heptadecanoic acid or a composition comprising heptadecanoic acid in said non-human organism or in the culture medium surrounding said non-human organism.

A further embodiment of the present invention relates to a process for the production of hexadecatrienoic acid, which comprises
(a) increasing or generating one or more activities selected from the group consisting of b1346-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of lauric acid, which comprises
(a) increasing or generating one or more activities selected from the group consisting of phage-related repressor protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lauric acid or a composition comprising lauric acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of lipoprotein precursor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of hexadecatrienoic acid, which comprises
(a) increasing or generating one or more activities selected from the group consisting of b1470-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of hexadecatrienoic acid, which comprises
(a) increasing or generating one or more activities selected from the group consisting of NADP-dependent malic enzyme in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxybehenic acid, which comprises
(a) increasing or generating one or more activities selected from the group consisting of b1522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of hexadecatrienoic acid, which comprises
(a) increasing or generating one or more activities selected from the group consisting of acid shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of phosphotransferase system component in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxybehenic acid, which comprises
(a) increasing or generating one or more activities selected from the group consisting of electron transport complex protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of hexadecatrienoic acid, which comprises
(a) increasing or generating one or more activities selected from the group consisting of pyridoxamine kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of b1670-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of hexadecadienoic acid, which comprises
(a) increasing or generating one or more activities selected from the group consisting of b1703-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecatrienoic acid, which comprises
(a) increasing or generating one or more activities selected from the group consisting of b1703-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of hexadecadienoic acid, which comprises
(a) increasing or generating one or more activities selected from the group consisting of transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecadienoic acid, which comprises
(a) increasing or generating one or more activities selected from the group consisting of b1837-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecatrienoic acid, which comprises
(a) increasing or generating one or more activities selected from the group consisting of b1837-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of hexadecadienoic acid, which comprises
(a) increasing or generating one or more activities selected from the group consisting of b2032-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecadienoic acid, which comprises
(a) increasing or generating one or more activities selected from the group consisting of b2099-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecatrienoic acid, which comprises
(a) increasing or generating one or more activities selected from the group consisting of b2099-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of b2107-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of lauric acid, which comprises
(a) increasing or generating one or more activities selected from the group consisting of b2126-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lauric acid or a composition comprising lauric acid in said non-human organism or in the culture medium surrounding said non-human organism.

A further embodiment of the present invention relates to a process for the production of 2-hydroxypalmitic acid, which comprises
(a) increasing or generating one or more activities selected from the group consisting of b2139-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of hexadecatrienoic acid, which comprises
(a) increasing or generating one or more activities selected from the group consisting of ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxybehenic acid, which comprises
(a) increasing or generating one or more activities selected from the group consisting of sensor kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of b2360-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of cysteine synthase A in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of hexadecadienoic acid, which comprises
(a) increasing or generating one or more activities selected from the group consisting of ethanolamine utilization protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecatrienoic acid, which comprises
(a) increasing or generating one or more activities selected from the group consisting of ethanolamine utilization protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of oleic acid, which comprises
(a) increasing or generating one or more activities selected from the group consisting of ethanolamine utilization protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

A further embodiment of the present invention relates to a process for the production of cerebronic acid, which comprises
(a) increasing or generating one or more activities selected from the group consisting of b2474-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of hexadecatrienoic acid, which comprises
(a) increasing or generating one or more activities selected from the group consisting of b2474-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of b2474-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of hexadecadienoic acid, which comprises
(a) increasing or generating one or more activities selected from the group consisting of b2513-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecadienoic acid, which comprises
(a) increasing or generating one or more activities selected from the group consisting of phosphatidylserine synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of b2654-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human

A furthogenribardiment of the present invention relates to a process for the production of hexadecatrienoic acid, which comprises
(a) increasing or generating one or more activities selected from the group consisting of 2-C-methyl-D-erythritol 4-phosphate cytidylyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of eicosanoic acid, which comprises
(a) increasing or generating one or more activities selected from the group consisting of alkaline phosphatase isozyme conversion protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of eicosanoic acid or a composition comprising eicosanoic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of murein transglycosylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of hexadecadienoic acid, which comprises
(a) increasing or generating one or more activities selected from the group consisting of b3442-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of cerebronic acid, which comprises
(a) increasing or generating one or more activities selected from the group consisting of sugar transport system permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of 2-hydroxybehenic acid, which comprises
(a) increasing or generating one or more activities selected from the group consisting of b3812-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of hexadecadienoic acid, which comprises
(a) increasing or generating one or more activities selected from the group consisting of 3-ketoacyl-CoA thiolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of oleic acid, which comprises
(a) increasing or generating one or more activities selected from the group consisting of 3-ketoacyl-CoA thiolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

A further embodiment of the present invention relates to a process for the production of hexadecadienoic acid, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecadienoic acid, which comprises
(a) increasing or generating one or more activities selected from the group consisting of fructose 1,6-bisphosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of behenic acid, which comprises
(a) increasing or generating one or more activities selected from the group consisting of cystathionine gamma-synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of behenic acid or a composition comprising behenic acid in said non-human organism or in the culture medium surrounding said non-human organism.

A further embodiment of the present invention relates to a process for the production of heptadecanoic acid, which comprises
(a) increasing or generating one or more activities selected from the group consisting of cystathionine gamma-synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of heptadecanoic acid or a composition comprising heptadecanoic acid in said non-human organism or in the culture medium surrounding said non-human organism.

A further embodiment of the present invention relates to a process for the production of oleic acid, which comprises
(a) increasing or generating one or more activities selected from the group consisting of cystathionine gamma-synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

A further embodiment of the present invention relates to a process for the production of 2-hydroxypalmitic acid, which comprises
(a) increasing or generating one or more activities selected from the group consisting of b3944-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of 2-hydroxybehenic acid, which comprises
(a) increasing or generating one or more activities selected from the group consisting of ankyrin repeat protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of hexadecadienoic acid, which comprises
(a) increasing or generating one or more activities selected from the group consisting of b4023-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of heptadecanoic acid, which comprises
(a) increasing or generating one or more activities selected from the group consisting of acetyl-coenzyme A synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of heptadecanoic acid or a composition comprising heptadecanoic acid in said non-human organism or in the culture medium surrounding said non-human organism.

A further embodiment of the present invention relates to a process for the production of 2-hydroxypalmitic acid, which comprises
(a) increasing or generating one or more activities selected from the group consisting of b4205-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of 2-hydroxybehenic acid, which comprises
(a) increasing or generating one or more activities selected from the group consisting of vacuolar processing enzyme in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxypalmitic acid, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of cerebronic acid, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of hexadecatrienoic acid, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of GM02LC38418-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of hexadecadienoic acid, which comprises
(a) increasing or generating one or more activities selected from the group consisting of sll0281-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of bifunctional protein (phosphoribosyltransferase and regulatory protein) in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of sll0406-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of cerebronic acid, which comprises
(a) increasing or generating one or more activities selected from the group consisting of binding-protein-dependent transport systems inner membrane component in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of cerebronic acid, which comprises
(a) increasing or generating one or more activities selected from the group consisting of phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of hexadecadienoic acid, which comprises
(a) increasing or generating one or more activities selected from the group consisting of carbon dioxide concentrating mechanism protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecatrienoic acid, which comprises
(a) increasing or generating one or more activities selected from the group consisting of carbon dioxide concentrating mechanism protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of cerebronic acid, which comprises
(a) increasing or generating one or more activities selected from the group consisting of coproporphyrinogen III oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of coproporphyrinogen III oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxypalmitic acid, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of hexadecatrienoic acid, which comprises
(a) increasing or generating one or more activities selected from the group consisting of bifunctional purine biosynthesis protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of cerebronic acid, which comprises
(a) increasing or generating one or more activities selected from the group consisting of malate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of hexadecatrienoic acid, which comprises
(a) increasing or generating one or more activities selected from the group consisting of glycine cleavage system H protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of permease protein of ABC transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of hexadecadienoic acid, which comprises
(a) increasing or generating one or more activities selected from the group consisting of tryptophan synthase alpha chain in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of 2-hydroxybehenic acid, which comprises
(a) increasing or generating one or more activities selected from the group consisting of dihydrolipoamide dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxypalmitic acid, which comprises
(a) increasing or generating one or more activities selected from the group consisting of phosphate import ATP-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of hexadecatrienoic acid, which comprises
(a) increasing or generating one or more activities selected from the group consisting of glutamate 5-kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of cerebronic acid, which comprises
(a) increasing or generating one or more activities selected from the group consisting of threonine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of hexadecatrienoic acid, which comprises
(a) increasing or generating one or more activities selected from the group consisting of threonine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of threonine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of ferredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxybehenic acid, which comprises
(a) increasing or generating one or more activities selected from the group consisting of Sec-independent protein translocase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of amino acid ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of cerebronic acid, which comprises
(a) increasing or generating one or more activities selected from the group consisting of metal-dependent hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of hexadecadienoic acid, which comprises
(a) increasing or generating one or more activities selected from the group consisting of metal-dependent hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of 2-hydroxybehenic acid, which comprises
(a) increasing or generating one or more activities selected from the group consisting of multiple antibiotic resistance protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of hexadecatrienoic acid, which comprises
(a) increasing or generating one or more activities selected from the group consisting of TTC1386-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxybehenic acid, which comprises
(a) increasing or generating one or more activities selected from the group consisting of homocitrate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of hexadecadienoic acid, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecatrienoic acid, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of hexadecadienoic acid, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecatrienoic acid, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of hexadecatrienoic acid, which comprises
(a) increasing or generating one or more activities selected from the group consisting of cell division control protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of cerebronic acid, which comprises
(a) increasing or generating one or more activities selected from the group consisting of ycl027w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of ycl027w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxypalmitic acid, which comprises
(a) increasing or generating one or more activities selected from the group consisting of ycl049c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of oleic acid, which comprises
(a) increasing or generating one or more activities selected from the group consisting of glycerol-3-phosphate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

A further embodiment of the present invention relates to a process for the production of eicosanoic acid, which comprises
(a) increasing or generating one or more activities selected from the group consisting of isocitrate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of eicosanoic acid or a composition comprising eicosanoic acid in said non-human organism or in the culture medium surrounding said non-human organism.

A further embodiment of the present invention relates to a process for the production of heptadecanoic acid, which comprises
(a) increasing or generating one or more activities selected from the group consisting of isocitrate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of heptadecanoic acid or a composition comprising heptadecanoic acid in said non-human organism or in the culture medium surrounding said non-human organism.

A further embodiment of the present invention relates to a process for the production of hexadecadienoic acid, which comprises
(a) increasing or generating one or more activities selected from the group consisting of cell division control protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecatrienoic acid, which comprises
(a) increasing or generating one or more activities selected from the group consisting of cell division control protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxybehenic acid, which comprises
(a) increasing or generating one or more activities selected from the group consisting of homocitrate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of behenic acid, which comprises
(a) increasing or generating one or more activities selected from the group consisting of phospho-2-dehydro-3-deoxyheptonate aldolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of behenic acid or a composition comprising behenic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of cell wall protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of F-box protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of cerebronic acid, which comprises
(a) increasing or generating one or more activities selected from the group consisting of ydr273w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of ydr273w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of hexadecatrienoic acid, which comprises
(a) increasing or generating one or more activities selected from the group consisting of ydr339c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of heptadecanoic acid, which comprises
(a) increasing or generating one or more activities selected from the group consisting of aldolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of heptadecanoic acid or a composition comprising heptadecanoic acid in said non-human organism or in the culture medium surrounding said non-human organism.

A further embodiment of the present invention relates to a process for the production of myristic acid, which comprises
(a) increasing or generating one or more activities selected from the group consisting of small nuclear ribonucleoprotein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myristic acid or a composition comprising myristic acid in said non-human organism or in the culture medium surrounding said non-human organism.

A further embodiment of the present invention relates to a process for the production of hexadecadienoic acid, which comprises
(a) increasing or generating one or more activities selected from the group consisting of molecular chaperone portein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecatrienoic acid, which comprises
(a) increasing or generating one or more activities selected from the group consisting of molecular chaperone portein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxybehenic acid, which comprises
(a) increasing or generating one or more activities selected from the group consisting of YFL019C-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxypalmitic acid, which comprises
(a) increasing or generating one or more activities selected from the group consisting of YFL019C-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of hexadecadienoic acid, which comprises
(a) increasing or generating one or more activities selected from the group consisting of aminotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecatrienoic acid, which comprises
(a) increasing or generating one or more activities selected from the group consisting of ygl237c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of eicosanoic acid, which comprises
(a) increasing or generating one or more activities selected from the group consisting of YGR104C-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of eicosanoic acid or a composition comprising eicosanoic acid in said non-human organism or in the culture medium surrounding said non-human organism.

A further embodiment of the present invention relates to a process for the production of cerebronic acid, which comprises
(a) increasing or generating one or more activities selected from the group consisting of squalene monooxygenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of hexadecadienoic acid, which comprises
(a) increasing or generating one or more activities selected from the group consisting of biotin synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of cerebronic acid, which comprises
(a) increasing or generating one or more activities selected from the group consisting of yhr033w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of cerebronic acid, which comprises
(a) increasing or generating one or more activities selected from the group consisting of DnaJ-like chaperone in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

A further embodiment of the present invention relates to a process for the production of 2-hydroxybehenic acid, which comprises
(a) increasing or generating one or more activities selected from the group consisting of YJL127W-A-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of cystathionine gamma-synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxypalmitic acid, which comprises
(a) increasing or generating one or more activities selected from the group consisting of cystathionine gamma-synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of 2-hydroxypalmitic acid, which comprises
(a) increasing or generating one or more activities selected from the group consisting of polygalacturonase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of hexadecatrienoic acid, which comprises
(a) increasing or generating one or more activities selected from the group consisting of cyclin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of hexadecadienoic acid, which comprises
(a) increasing or generating one or more activities selected from the group consisting of ylr326w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

A further embodiment of the present invention relates to a process for the production of hexadecatrienoic acid, which comprises
(a) increasing or generating one or more activities selected from the group consisting of ylr326w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxybehenic acid, which comprises
(a) increasing or generating one or more activities selected from the group consisting of yml083c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of yml084w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human

A furthangenribardiment of the present invention relates to a process for the production of 2-hydroxypalmitic acid, which comprises
(a) increasing or generating one or more activities selected from the group consisting of molecular chaperone in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of oleic acid, which comprises
(a) increasing or generating one or more activities selected from the group consisting of phosphotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

A further embodiment of the present invention relates to a process for the production of 2-hydroxybehenic acid, which comprises
(a) increasing or generating one or more activities selected from the group consisting of ynl321w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of hexadecatrienoic acid, which comprises
(a) increasing or generating one or more activities selected from the group consisting of Elongation factor Tu in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a) increasing or generating one or more activities selected from the group consisting of ATPase epsilon subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

A further embodiment of the present invention relates to a process for the production of 2-hydroxypalmitic acid, which comprises
(a) increasing or generating one or more activities selected from the group consisting of phosphoadenosine phosphosulfate reductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

A further embodiment of the present invention relates to a process for the production of oleic acid, which comprises
(a) increasing or generating one or more activities selected from the group consisting of 40S ribosomal protein S17 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

A further embodiment of the present invention relates to a process for the production of 2-hydroxybehenic acid, which comprises
(a) increasing or generating one or more activities selected from the group consisting of b3644-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

A further embodiment of the present invention relates to a process for the production of behenic acid, which comprises
(a) increasing or generating one or more activities selected from the group consisting of b3644-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of behenic acid or a composition comprising behenic acid in said non-human organism or in the culture medium surrounding said non-human organism.

A further embodiment of the present invention relates to a process for the production of nervonic acid, which comprises
(a) increasing or generating one or more activities selected from the group consisting of b3644-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of nervonic acid or a composition comprising nervonic acid in said non-human organism or in the culture medium surrounding said non-human organism.

A further embodiment of the present invention relates to a process for the production of 2-hydroxybehenic acid, which comprises
(a1) increasing or generating the activity of a polypeptide comprising a polypeptide as depicted in the respective line in column 5 or 8 of Table II, application no. 12, or a homolog or fragment thereof, a consensus sequence or at least one polypeptide motif as depicted in column 8 of Table IV, application no. 12, whereby the respective line discloses in column 7 the fine chemical 2-hydroxybehenic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical 2-hydroxybehenic acid;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, which is joined to a transit peptide, whereby the respective line discloses in column 7 the fine chemical 2-hydroxybehenic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line discloses in column 7 the fine chemical 2-hydroxybehenic acid;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical 2-hydroxybehenic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical 2-hydroxybehenic acid;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of 2-hydroxybehenic acid, or a composition comprising 2-hydroxybehenic acid in said non-human organism or in the culture medium surrounding said non-human organism.

A further embodiment of the present invention relates to a process for the production of 2-hydroxypalmitic acid, which comprises
(a1) increasing or generating the activity of a polypeptide comprising a polypeptide as depicted in the respective line in column 5 or 8 of Table II, application no. 12, or a homolog or fragment thereof, a consensus sequence or at least one polypeptide motif as depicted in column 8 of Table IV, application no. 12, whereby the respective line discloses in column 7 the fine chemical 2-hydroxypalmitic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical 2-hydroxypalmitic acid;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, which is joined to a transit peptide, whereby the respective line discloses in column 7 the fine chemical 2-hydroxypalmitic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line discloses in column 7 the fine chemical 2-hydroxypalmitic acid;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical 2-hydroxypalmitic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical 2-hydroxypalmitic acid;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of 2-hydroxypalmitic acid, or a composition comprising 2-hydroxypalmitic acid in said non-human organism or in the culture medium surrounding said non-human organism.

A further embodiment of the present invention relates to a process for the production of 2-hydroxytetracosenoic acid (preferably 2-hydroxynervonic acid), which comprises
(a1) increasing or generating the activity of a polypeptide comprising a polypeptide as depicted in the respective line in column 5 or 8 of Table II, application no. 12, or a homolog or fragment thereof, a consensus sequence or at least one polypeptide motif as depicted in column 8 of Table IV, application no. 12, whereby the respective line discloses in column 7 the fine chemical 2-hydroxytetracosenoic acid (preferably 2-hydroxynervonic acid); or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical 2-hydroxytetracosenoic acid (preferably 2-hydroxynervonic acid);
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, which is joined to a transit peptide, whereby the respective line discloses in column 7 the fine chemical 2-hydroxytetracosenoic acid (preferably 2-hydroxynervonic acid); or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line discloses in column 7 the fine chemical 2-hydroxytetracosenoic acid (preferably 2-hydroxynervonic acid);
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical 2-hydroxytetracosenoic acid (preferably 2-hydroxynervonic acid); or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical 2-hydroxytetracosenoic acid (preferably 2-hydroxynervonic acid);
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of 2-hydroxytetracosenoic acid (preferably 2-hydroxynervonic acid), or a composition comprising 2-hydroxytetracosenoic acid (preferably 2-hydroxynervonic acid) in said non-human organism or in the culture medium surrounding said non-human organism.

A further embodiment of the present invention relates to a process for the production of behenic acid, which comprises
(a1) increasing or generating the activity of a polypeptide comprising a polypeptide as depicted in the respective line in column 5 or 8 of Table II, application no. 12, or a homolog or fragment thereof, a consensus sequence or at least one polypeptide motif as depicted in column 8 of Table IV, application no. 12, whereby the respective line discloses in column 7 the fine chemical behenic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical behenic acid;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, which is joined to a transit peptide, whereby the respective line discloses in column 7 the fine chemical behenic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line discloses in column 7 the fine chemical behenic acid;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical behenic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical behenic acid;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of behenic acid, or a composition comprising behenic acid in said non-human organism or in the culture medium surrounding said non-human organism.

A further embodiment of the present invention relates to a process for the production of cerebronic acid, which comprises
(a1) increasing or generating the activity of a polypeptide comprising a polypeptide as depicted in the respective line in column 5 or 8 of Table II, application no. 12, or a homolog or fragment thereof, a consensus sequence or at least one polypeptide motif as depicted in column 8 of Table IV, application no. 12, whereby the respective line discloses in column 7 the fine chemical cerebronic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical cerebronic acid;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, which is joined to a transit peptide, whereby the respective line discloses in column 7 the fine chemical cerebronic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line discloses in column 7 the fine chemical cerebronic acid;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical cerebronic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical cerebronic acid;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of cerebronic acid, or a composition comprising cerebronic acid in said non-human organism or in the culture medium surrounding said non-human organism.

A further embodiment of the present invention relates to a process for the production of eicosanoic acid, which comprises
(a1) increasing or generating the activity of a polypeptide comprising a polypeptide as depicted in the respective line in column 5 or 8 of Table II, application no. 12, or a homolog or fragment thereof, a consensus sequence or at least one polypeptide motif as depicted in column 8 of Table IV, application no. 12, whereby the respective line discloses in column 7 the fine chemical eicosanoic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical eicosanoic acid;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, which is joined to a transit peptide, whereby the respective line discloses in column 7 the fine chemical eicosanoic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line discloses in column 7 the fine chemical eicosanoic acid;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical eicosanoic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical eicosanoic acid;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of eicosanoic acid, or a composition comprising eicosanoic acid in said non-human organism or in the culture medium surrounding said non-human organism.

A further embodiment of the present invention relates to a process for the production of heptadecanoic acid, which comprises
(a1) increasing or generating the activity of a polypeptide comprising a polypeptide as depicted in the respective line in column 5 or 8 of Table II, application no. 12, or a homolog or fragment thereof, a consensus sequence or at least one polypeptide motif as depicted in column 8 of Table IV, application no. 12, whereby the respective line discloses in column 7 the fine chemical heptadecanoic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical heptadecanoic acid;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, which is joined to a transit peptide, whereby the respective line discloses in column 7 the fine chemical heptadecanoic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line discloses in column 7 the fine chemical heptadecanoic acid;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical heptadecanoic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical heptadecanoic acid;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of heptadecanoic acid, or a composition comprising heptadecanoic acid in said non-human organism or in the culture medium surrounding said non-human organism.

A further embodiment of the present invention relates to a process for the production of hexadecadienoic acid, which comprises
(a1) increasing or generating the activity of a polypeptide comprising a polypeptide as depicted in the respective line in column 5 or 8 of Table II, application no. 12, or a homolog or fragment thereof, a consensus sequence or at least one polypeptide motif as depicted in column 8 of Table IV, application no. 12, whereby the respective line discloses in column 7 the fine chemical hexadecadienoic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical hexadecadienoic acid;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, which is joined to a transit peptide, whereby the respective line discloses in column 7 the fine chemical hexadecadienoic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line discloses in column 7 the fine chemical hexadecadienoic acid;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical hexadecadienoic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical hexadecadienoic acid;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of hexadecadienoic acid, or a composition comprising hexadecadienoic acid in said non-human organism or in the culture medium surrounding said non-human organism.

A further embodiment of the present invention relates to a process for the production of hexadecatrienoic acid, which comprises
(a1) increasing or generating the activity of a polypeptide comprising a polypeptide as depicted in the respective line in column 5 or 8 of Table II, application no. 12, or a homolog or fragment thereof, a consensus sequence or at least one polypeptide motif as depicted in column 8 of Table IV, application no. 12, whereby the respective line discloses
in column 7 the fine chemical hexadecatrienoic acid; or increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical hexadecatrienoic acid;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, which is joined to a transit peptide, whereby the respective line discloses in column 7 the fine chemical hexadecatrienoic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line discloses in column 7 the fine chemical hexadecatrienoic acid;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical hexadecatrienoic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical hexadecatrienoic acid;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of hexadecatrienoic acid, or a composition comprising hexadecatrienoic acid in said non-human organism or in the culture medium surrounding said non-human organism.

A further embodiment of the present invention relates to a process for the production of lauric acid, which comprises
(a1) increasing or generating the activity of a polypeptide comprising a polypeptide as depicted in the respective line in column 5 or 8 of Table II, application no. 12, or a homolog or fragment thereof, a consensus sequence or at least one polypeptide motif as depicted in column 8 of Table IV, application no. 12, whereby the respective line discloses in column 7 the fine chemical lauric acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical lauric acid;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, which is joined to a transit peptide, whereby the respective line discloses in column 7 the fine chemical lauric acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line discloses in column 7 the fine chemical lauric acid;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical lauric acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical lauric acid;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of lauric acid, or a composition comprising lauric acid in said non-human organism or in the culture medium surrounding said non-human organism.

A further embodiment of the present invention relates to a process for the production of myristic acid, which comprises
(a1) increasing or generating the activity of a polypeptide comprising a polypeptide as depicted in the respective line in column 5 or 8 of Table II, application no. 12, or a homolog or fragment thereof, a consensus sequence or at least one polypeptide motif as depicted in column 8 of Table IV, application no. 12, whereby the respective line discloses in column 7 the fine chemical myristic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical myristic acid;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, which is joined to a transit peptide, whereby the respective line discloses in column 7 the fine chemical myristic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line discloses in column 7 the fine chemical myristic acid;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical myristic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical myristic acid;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of myristic acid, or a composition comprising myristic acid in said non-human organism or in the culture medium surrounding said non-human organism.

A further embodiment of the present invention relates to a process for the production of nervonic acid, which comprises
(a1) increasing or generating the activity of a polypeptide comprising a polypeptide as depicted in the respective line in column 5 or 8 of Table II, application no. 12, or a homolog or fragment thereof, a consensus sequence or at least one polypeptide motif as depicted in column 8 of Table IV, application no. 12, whereby the respective line discloses in column 7 the fine chemical nervonic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical nervonic acid;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, which is joined to a transit peptide, whereby the respective line discloses in column 7 the fine chemical nervonic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line discloses in column 7 the fine chemical nervonic acid;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical nervonic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical nervonic acid;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of nervonic acid, or a composition comprising nervonic acid in said non-human organism or in the culture medium surrounding said non-human organism.

A further embodiment of the present invention relates to a process for the production of oleic acid, which comprises
(a1) increasing or generating the activity of a polypeptide comprising a polypeptide as depicted in the respective line in column 5 or 8 of Table II, application no. 12, or a homolog or fragment thereof, a consensus sequence or at least one polypeptide motif as depicted in column 8 of Table IV, application no. 12, whereby the respective line discloses in column 7 the fine chemical oleic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical oleic acid;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, which is joined to a transit peptide, whereby the respective line discloses in column 7 the fine chemical oleic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line discloses in column 7 the fine chemical oleic acid;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical oleic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical oleic acid;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of oleic acid, or a composition comprising oleic acid in said non-human organism or in the culture medium surrounding said non-human organism.

Accordingly, the present invention relates to a process for the production of cerebronic acid, which comprises
(a) increasing or generating the activity of a chlorophyllase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a chlorophyllase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of hexadecatrienoic acid, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of cerebronic acid, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of hexadecatrienoic acid, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxybehenic acid, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxypalmitic acid, which comprises
(a) increasing or generating the activity of a At2g33510-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of cerebronic acid, which comprises
(a) increasing or generating the activity of a adenosine kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of 2-hydroxybehenic acid, which comprises
(a) increasing or generating the activity of a adenosine kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a DNA mismatch repair protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of cerebronic acid, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of hexadecatrienoic acid, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxybehenic acid, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxypalmitic acid, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of 2-hydroxybehenic acid, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxypalmitic acid, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of hexadecatrienoic acid, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxybehenic acid, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of cerebronic acid, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of 2-hydroxybehenic acid, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of cerebronic acid, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of hexadecadienoic acid, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecatrienoic acid, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxybehenic acid, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxypalmitic acid, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of oleic acid, which comprises
(a) increasing or generating the activity of a acyl-desaturase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

Accordingly, the present invention relates to a process for the production of hexadecatrienoic acid, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of hexadecatrienoic acid, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of hexadecadienoic acid, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of cerebronic acid, which comprises
(a) increasing or generating the activity of a CDP-diacylglycerol-serine O-phosphatidyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of 2-hydroxybehenic acid, which comprises
(a) increasing or generating the activity of a CDP-diacylglycerol-serine O-phosphatidyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a CDP-diacylglycerol-serine O-phosphatidyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxypalmitic acid, which comprises
(a) increasing or generating the activity of a CDP-diacylglycerol-serine O-phosphatidyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of hexadecadienoic acid, which comprises
(a) increasing or generating the activity of a sec-independent protein translocase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a transcriptional regulator protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxypalmitic acid, which comprises
(a) increasing or generating the activity of a beta-hydroxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of hexadecadienoic acid, which comprises
(a) increasing or generating the activity of a 30S ribosomal protein S3 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of 2-hydroxybehenic acid, which comprises
(a) increasing or generating the activity of a acyl-CoA synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a heat shock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of hexadecadienoic acid, which comprises
(a) increasing or generating the activity of a recombinase A in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of lauric acid, which comprises
(a) increasing or generating the activity of a ABC transporter component in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lauric acid or a composition comprising lauric acid in said non-human organism or in the culture medium surrounding said non-human organism.

Accordingly, the present invention relates to a process for the production of 2-hydroxypalmitic acid, which comprises
(a) increasing or generating the activity of a hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of 2-hydroxypalmitic acid, which comprises
(a) increasing or generating the activity of a glutamate-ammonia-ligase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of 2-hydroxybehenic acid, which comprises
(a) increasing or generating the activity of a fumarylacetoacetate hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a N-acetyl-gamma-glutamyl-phosphate reductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a HesB/YadR/YfhF family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a purine nucleoside phosphorylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human

Accord ivp,iAhre present invention relates to a process for the production of 2-hydroxybehenic acid, which comprises
(a) increasing or generating the activity of a b0050-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a b0050-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of cerebronic acid, which comprises
(a) increasing or generating the activity of a b0249-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of behenic acid, which comprises
(a) increasing or generating the activity of a betaine aldehyde dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of behenic acid or a composition comprising behenic acid in said non-human organism or in the culture medium surrounding said non-human organism.

Accordingly, the present invention relates to a process for the production of eicosanoic acid, which comprises
(a) increasing or generating the activity of a betaine aldehyde dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of eicosanoic acid or a composition comprising eicosanoic acid in said non-human organism or in the culture medium surrounding said non-human organism.

Accordingly, the present invention relates to a process for the production of myristic acid, which comprises
(a) increasing or generating the activity of a 2,3-dihydroxyphenylpropionate 1,2-dioxygenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myristic acid or a composition comprising myristic acid in said non-human organism or in the culture medium surrounding said non-human organism.

Accordingly, the present invention relates to a process for the production of oleic acid, which comprises
(a) increasing or generating the activity of a 2,3-dihydroxyphenylpropionate 1,2-dioxygenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

Accordingly, the present invention relates to a process for the production of hexadecatrienoic acid, which comprises
(a) increasing or generating the activity of a b0371-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of cerebronic acid, which comprises
(a) increasing or generating the activity of a ATP-binding component of a transport system in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of 2-hydroxybehenic acid, which comprises
(a) increasing or generating the activity of a ATP-binding component of a transport system in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxypalmitic acid, which comprises
(a) increasing or generating the activity of a ATP-binding component of a transport system in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of hexadecadienoic acid, which comprises
(a) increasing or generating the activity of a b0518-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of 2-hydroxybehenic acid, which comprises
(a) increasing or generating the activity of a sensor histidine kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a sensor histidine kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of cerebronic acid, which comprises
(a) increasing or generating the activity of a citrate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a b0801-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of cerebronic acid, which comprises
(a) increasing or generating the activity of a b1046-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of hexadecatrienoic acid, which comprises
(a) increasing or generating the activity of a malonyl CoA-acyl carrier protein transacylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of hexadecadienoic acid, which comprises
(a) increasing or generating the activity of a 3-oxoacyl-[acyl-carrier-protein] reductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecatrienoic acid, which comprises
(a) increasing or generating the activity of a 3-oxoacyl-[acyl-carrier-protein] reductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of hexadecatrienoic acid, which comprises
(a) increasing or generating the activity of a 3-oxoacyl-[acyl-carrier-protein] synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxybehenic acid, which comprises
(a) increasing or generating the activity of a 3-oxoacyl-[acyl-carrier-protein] synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of oleic acid, which comprises
(a) increasing or generating the activity of a sodium/proton antiporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

Accordingly, the present invention relates to a process for the production of 2-hydroxypalmitic acid, which comprises
(a) increasing or generating the activity of a sodium/proton antiporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of behenic acid, which comprises
(a) increasing or generating the activity of a outer-membrane lipoprotein precursor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of behenic acid or a composition comprising behenic acid in said non-human organism or in the culture medium surrounding said non-human organism.

Accordingly, the present invention relates to a process for the production of behenic acid, which comprises
(a) increasing or generating the activity of a cardiolipin synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of behenic acid or a composition comprising behenic acid in said non-human organism or in the culture medium surrounding said non-human organism.

Accordingly, the present invention relates to a process for the production of heptadecanoic acid, which comprises
(a) increasing or generating the activity of a cardiolipin synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of heptadecanoic acid or a composition comprising heptadecanoic acid in said non-human organism or in the culture medium surrounding said non-human organism.

Accordingly, the present invention relates to a process for the production of hexadecatrienoic acid, which comprises
(a) increasing or generating the activity of a b1346-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of lauric acid, which comprises
(a) increasing or generating the activity of a phage-related repressor protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lauric acid or a composition comprising lauric acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a lipoprotein precursor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of hexadecatrienoic acid, which comprises
(a) increasing or generating the activity of a b1470-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of hexadecatrienoic acid, which comprises
(a) increasing or generating the activity of a NADP-dependent malic enzyme in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxybehenic acid, which comprises
(a) increasing or generating the activity of a b1522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of hexadecatrienoic acid, which comprises
(a) increasing or generating the activity of a acid shock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a phosphotransferase system component in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxybehenic acid, which comprises
(a) increasing or generating the activity of a electron transport complex protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of hexadecatrienoic acid, which comprises
(a) increasing or generating the activity of a pyridoxamine kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a b1670-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of hexadecadienoic acid, which comprises
(a) increasing or generating the activity of a b1703-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecatrienoic acid, which comprises
(a) increasing or generating the activity of a b1703-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of hexadecadienoic acid, which comprises
(a) increasing or generating the activity of a transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecadienoic acid, which comprises
(a) increasing or generating the activity of a b1837-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecatrienoic acid, which comprises
(a) increasing or generating the activity of a b1837-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of hexadecadienoic acid, which comprises
(a) increasing or generating the activity of a b2032-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecadienoic acid, which comprises
(a) increasing or generating the activity of a b2099-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecatrienoic acid, which comprises
(a) increasing or generating the activity of a b2099-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a b2107-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of lauric acid, which comprises
(a) increasing or generating the activity of a b2126-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lauric acid or a composition comprising lauric acid in said non-human organism or in the culture medium surrounding said non-human organism.

Accordingly, the present invention relates to a process for the production of 2-hydroxypalmitic acid, which comprises
(a) increasing or generating the activity of a b2139-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of hexadecatrienoic acid, which comprises
(a) increasing or generating the activity of a ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxybehenic acid, which comprises
(a) increasing or generating the activity of a sensor kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a b2360-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a cysteine synthase A in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of hexadecadienoic acid, which comprises
(a) increasing or generating the activity of a ethanolamine utilization protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecatrienoic acid, which comprises
(a) increasing or generating the activity of a ethanolamine utilization protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of oleic acid, which comprises
(a) increasing or generating the activity of a ethanolamine utilization protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

Accordingly, the present invention relates to a process for the production of cerebronic acid, which comprises
(a) increasing or generating the activity of a b2474-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of hexadecatrienoic acid, which comprises
(a) increasing or generating the activity of a b2474-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a b2474-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of hexadecadienoic acid, which comprises
(a) increasing or generating the activity of a b2513-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecadienoic acid, which comprises
(a) increasing or generating the activity of a phosphatidylserine synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a b2654-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of hexadecatrienoic acid, which comprises
(a) increasing or generating the activity of a 2-C-methyl-D-erythritol 4-phosphate cytidylyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of eicosanoic acid, which comprises
(a) increasing or generating the activity of a alkaline phosphatase isozyme conversion protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of eicosanoic acid or a composition comprising eicosanoic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a murein transglycosylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of hexadecadienoic acid, which comprises
(a) increasing or generating the activity of a b3442-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of cerebronic acid, which comprises
(a) increasing or generating the activity of a sugar transport system permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of 2-hydroxybehenic acid, which comprises
(a) increasing or generating the activity of a b3812-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of hexadecadienoic acid, which comprises
(a) increasing or generating the activity of a 3-ketoacyl-CoA thiolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of oleic acid, which comprises
(a) increasing or generating the activity of a 3-ketoacyl-CoA thiolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

Accordingly, the present invention relates to a process for the production of hexadecadienoic acid, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecadienoic acid, which comprises
(a) increasing or generating the activity of a fructose 1,6-bisphosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of behenic acid, which comprises
(a) increasing or generating the activity of a cystathionine gamma-synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of behenic acid or a composition comprising behenic acid in said non-human organism or in the culture medium surrounding said non-human organism.

Accordingly, the present invention relates to a process for the production of heptadecanoic acid, which comprises
(a) increasing or generating the activity of a cystathionine gamma-synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of heptadecanoic acid or a composition comprising heptadecanoic acid in said non-human organism or in the culture medium surrounding said non-human organism.

Accordingly, the present invention relates to a process for the production of oleic acid, which comprises
(a) increasing or generating the activity of a cystathionine gamma-synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

Accordingly, the present invention relates to a process for the production of 2-hydroxypalmitic acid, which comprises
(a) increasing or generating the activity of a b3944-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of 2-hydroxybehenic acid, which comprises
(a) increasing or generating the activity of a ankyrin repeat protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of hexadecadienoic acid, which comprises
(a) increasing or generating the activity of a b4023-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of heptadecanoic acid, which comprises
(a) increasing or generating the activity of a acetyl-coenzyme A synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of heptadecanoic acid or a composition comprising heptadecanoic acid in said non-human organism or in the culture medium surrounding said non-human organism.

Accordingly, the present invention relates to a process for the production of 2-hydroxypalmitic acid, which comprises
(a) increasing or generating the activity of a b4205-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of 2-hydroxybehenic acid, which comprises
(a) increasing or generating the activity of a vacuolar processing enzyme in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxypalmitic acid, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of cerebronic acid, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of hexadecatrienoic acid, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a GM02LC38418-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of hexadecadienoic acid, which comprises
(a) increasing or generating the activity of a sll0281-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a bifunctional protein (phosphoribosyltransferase and regulatory protein) in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a sll0406-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of cerebronic acid, which comprises
(a) increasing or generating the activity of a binding-protein-dependent transport systems inner membrane component in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of cerebronic acid, which comprises
(a) increasing or generating the activity of a phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of hexadecadienoic acid, which comprises
(a) increasing or generating the activity of a carbon dioxide concentrating mechanism protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecatrienoic acid, which comprises
(a) increasing or generating the activity of a carbon dioxide concentrating mechanism protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of cerebronic acid, which comprises
(a) increasing or generating the activity of a coproporphyrinogen III oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a coproporphyrinogen III oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxypalmitic acid, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of hexadecatrienoic acid, which comprises
(a) increasing or generating the activity of a bifunctional purine biosynthesis protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of cerebronic acid, which comprises
(a) increasing or generating the activity of a malate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of hexadecatrienoic acid, which comprises
(a) increasing or generating the activity of a glycine cleavage system H protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a permease protein of ABC transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of hexadecadienoic acid, which comprises
(a) increasing or generating the activity of a tryptophan synthase alpha chain in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of 2-hydroxybehenic acid, which comprises
(a) increasing or generating the activity of a dihydrolipoamide dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxypalmitic acid, which comprises
(a) increasing or generating the activity of a phosphate import ATP-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of hexadecatrienoic acid, which comprises
(a) increasing or generating the activity of a glutamate 5-kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of cerebronic acid, which comprises
(a) increasing or generating the activity of a threonine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of hexadecatrienoic acid, which comprises
(a) increasing or generating the activity of a threonine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a threonine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a ferredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human

Accord ivp,iArre present invention relates to a process for the production of 2-hydroxybehenic acid, which comprises
(a) increasing or generating the activity of a Sec-independent protein translocase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a amino acid ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of cerebronic acid, which comprises
(a) increasing or generating the activity of a metal-dependent hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of hexadecadienoic acid, which comprises
(a) increasing or generating the activity of a metal-dependent hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of 2-hydroxybehenic acid, which comprises
(a) increasing or generating the activity of a multiple antibiotic resistance protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of hexadecatrienoic acid, which comprises
(a) increasing or generating the activity of a TTC1386-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxybehenic acid, which comprises
(a) increasing or generating the activity of a homocitrate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of hexadecadienoic acid, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecatrienoic acid, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of hexadecadienoic acid, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecatrienoic acid, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of hexadecatrienoic acid, which comprises
(a) increasing or generating the activity of a cell division control protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of cerebronic acid, which comprises
(a) increasing or generating the activity of a ycl027w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a ycl027w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxypalmitic acid, which comprises
(a) increasing or generating the activity of a ycl049c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of oleic acid, which comprises
(a) increasing or generating the activity of a glycerol-3-phosphate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

Accordingly, the present invention relates to a process for the production of eicosanoic acid, which comprises
(a) increasing or generating the activity of a isocitrate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of eicosanoic acid or a composition comprising eicosanoic acid in said non-human organism or in the culture medium surrounding said non-human organism.

Accordingly, the present invention relates to a process for the production of heptadecanoic acid, which comprises
(a) increasing or generating the activity of a isocitrate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of heptadecanoic acid or a composition comprising heptadecanoic acid in said non-human organism or in the culture medium surrounding said non-human organism.

Accordingly, the present invention relates to a process for the production of hexadecadienoic acid, which comprises
(a) increasing or generating the activity of a cell division control protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecatrienoic acid, which comprises
(a) increasing or generating the activity of a cell division control protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxybehenic acid, which comprises
(a) increasing or generating the activity of a homocitrate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of behenic acid, which comprises
(a) increasing or generating the activity of a phospho-2-dehydro-3-deoxyheptonate aldolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of behenic acid or a composition comprising behenic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a cell wall protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a F-box protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of cerebronic acid, which comprises
(a) increasing or generating the activity of a ydr273w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a ydr273w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human

Accordirgjip,iAhre present invention relates to a process for the production of hexadecatrienoic acid, which comprises
(a) increasing or generating the activity of a ydr339c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of heptadecanoic acid, which comprises
(a) increasing or generating the activity of a aldolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of heptadecanoic acid or a composition comprising heptadecanoic acid in said non-human organism or in the culture medium surrounding said non-human organism.

Accordingly, the present invention relates to a process for the production of myristic acid, which comprises
(a) increasing or generating the activity of a small nuclear ribonucleoprotein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myristic acid or a composition comprising myristic acid in said non-human organism or in the culture medium surrounding said non-human organism.

Accordingly, the present invention relates to a process for the production of hexadecadienoic acid, which comprises
(a) increasing or generating the activity of a molecular chaperone portein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecatrienoic acid, which comprises
(a) increasing or generating the activity of a molecular chaperone portein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxybehenic acid, which comprises
(a) increasing or generating the activity of a YFL019C-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxypalmitic acid, which comprises
(a) increasing or generating the activity of a YFL019C-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of hexadecadienoic acid, which comprises
(a) increasing or generating the activity of a aminotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecatrienoic acid, which comprises
(a) increasing or generating the activity of a ygl237c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of eicosanoic acid, which comprises
(a) increasing or generating the activity of a YGR104C-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of eicosanoic acid or a composition comprising eicosanoic acid in said non-human organism or in the culture medium surrounding said non-human organism.

Accordingly, the present invention relates to a process for the production of cerebronic acid, which comprises
(a) increasing or generating the activity of a squalene monooxygenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of hexadecadienoic acid, which comprises
(a) increasing or generating the activity of a biotin synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of cerebronic acid, which comprises
(a) increasing or generating the activity of a yhr033w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of cerebronic acid, which comprises
(a) increasing or generating the activity of a DnaJ-like chaperone in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerebronic acid or a composition comprising cerebronic acid in said non-human organism or in the culture medium surrounding said non-human organism.

Accordingly, the present invention relates to a process for the production of 2-hydroxybehenic acid, which comprises
(a) increasing or generating the activity of a YJL127W-A-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a cystathionine gamma-synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxypalmitic acid, which comprises
(a) increasing or generating the activity of a cystathionine gamma-synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of 2-hydroxypalmitic acid, which comprises
(a) increasing or generating the activity of a polygalacturonase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of hexadecatrienoic acid, which comprises
(a) increasing or generating the activity of a cyclin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of hexadecadienoic acid, which comprises
(a) increasing or generating the activity of a ylr326w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecadienoic acid or a composition comprising hexadecadienoic acid in said non-human organism or in the culture medium surrounding said non-human organism.

Accordingly, the present invention relates to a process for the production of hexadecatrienoic acid, which comprises
(a) increasing or generating the activity of a ylr326w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxybehenic acid, which comprises
(a) increasing or generating the activity of a yml083c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a yml084w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxypalmitic acid, which comprises
(a) increasing or generating the activity of a molecular chaperone in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of oleic acid, which comprises
(a) increasing or generating the activity of a phosphotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

Accordingly, the present invention relates to a process for the production of 2-hydroxybehenic acid, which comprises
(a) increasing or generating the activity of a ynl321w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of hexadecatrienoic acid, which comprises
(a) increasing or generating the activity of a Elongation factor Tu in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of hexadecatrienoic acid or a composition comprising hexadecatrienoic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a ATPase epsilon subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxytetracosenoic acid (preferably 2-hydroxynervonic acid) or a composition comprising 2-hydroxytetracosenoic acid (preferably 2-hydroxynervonic acid) in said non-human organism or in the culture medium surrounding said non-human organism.

Accordingly, the present invention relates to a process for the production of 2-hydroxypalmitic acid, which comprises
(a) increasing or generating the activity of a phosphoadenosine phosphosulfate reductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxypalmitic acid or a composition comprising 2-hydroxypalmitic acid in said non-human organism or in the culture medium surrounding said non-human organism.

Accordingly, the present invention relates to a process for the production of oleic acid, which comprises
(a) increasing or generating the activity of a 40S ribosomal protein S17 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oleic acid or a composition comprising oleic acid in said non-human organism or in the culture medium surrounding said non-human organism.

Accordingly, the present invention relates to a process for the production of 2-hydroxybehenic acid, which comprises
(a) increasing or generating the activity of a b3644-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid or a composition comprising 2-hydroxybehenic acid in said non-human organism or in the culture medium surrounding said non-human organism.

Accordingly, the present invention relates to a process for the production of behenic acid, which comprises
(a) increasing or generating the activity of a b3644-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of behenic acid or a composition comprising behenic acid in said non-human organism or in the culture medium surrounding said non-human organism.

Accordingly, the present invention relates to a process for the production of nervonic acid, which comprises
(a) increasing or generating the activity of a b3644-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of nervonic acid or a composition comprising nervonic acid in said non-human organism or in the culture medium surrounding said non-human organism.

Accordingly, the present invention relates to a process for the production of 2-hydroxybehenic acid, which comprises
(a) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical 2-hydroxybehenic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical 2-hydroxybehenic acid;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of 2-hydroxybehenic acid, or a composition comprising 2-hydroxybehenic acid in said non-human organism or in the culture medium surrounding said non-human organism.

Accordingly, the present invention relates to a process for the production of 2-hydroxypalmitic acid, which comprises
(a) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical 2-hydroxypalmitic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical 2-hydroxypalmitic acid;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of 2-hydroxypalmitic acid, or a composition comprising 2-hydroxypalmitic acid in said non-human organism or in the culture medium surrounding said non-human organism.

Accordingly, the present invention relates to a process for the production of 2-hydroxytetracosenoic acid (preferably 2-hydroxynervonic acid), which comprises
(a) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical 2-hydroxytetracosenoic acid (preferably 2-hydroxynervonic acid); or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical 2-hydroxytetracosenoic acid (preferably 2-hydroxynervonic acid);
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of 2-hydroxytetracosenoic acid (preferably 2-hydroxynervonic acid), or a composition comprising 2-hydroxytetracosenoic acid (preferably 2-hydroxynervonic acid) in said non-human organism or in the culture medium surrounding said non-human organism.

Accordingly, the present invention relates to a process for the production of behenic acid, which comprises
(a) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical behenic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical behenic acid;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of behenic acid, or a composition comprising behenic acid in said non-human organism or in the culture medium surrounding said non-human organism.

Accordingly, the present invention relates to a process for the production of cerebronic acid, which comprises
(a) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical cerebronic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical cerebronic acid;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of cerebronic acid, or a composition comprising cerebronic acid in said non-human organism or in the culture medium surrounding said non-human organism.

Accordingly, the present invention relates to a process for the production of eicosanoic acid, which comprises
(a) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical eicosanoic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical eicosanoic acid;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of eicosanoic acid, or a composition comprising eicosanoic acid in said non-human organism or in the culture medium surrounding said non-human organism.

Accordingly, the present invention relates to a process for the production of heptadecanoic acid, which comprises
(a) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical heptadecanoic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical heptadecanoic acid;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of heptadecanoic acid, or a composition comprising heptadecanoic acid in said non-human organism or in the culture medium surrounding said non-human organism.

Accordingly, the present invention relates to a process for the production of hexadecadienoic acid, which comprises
(a) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical hexadecadienoic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical hexadecadienoic acid;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of hexadecadienoic acid, or a composition comprising hexadecadienoic acid in said non-human organism or in the culture medium surrounding said non-human organism.

Accordingly, the present invention relates to a process for the production of hexadecatrienoic acid, which comprises
(a) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical hexadecatrienoic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical hexadecatrienoic acid;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of hexadecatrienoic acid, or a composition comprising hexadecatrienoic acid in said non-human organism or in the culture medium surrounding said non-human organism.

Accordingly, the present invention relates to a process for the production of lauric acid, which comprises
(a) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical lauric acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical lauric acid;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of lauric acid, or a composition comprising lauric acid in said non-human organism or in the culture medium surrounding said non-human organism.

Accordingly, the present invention relates to a process for the production of myristic acid, which comprises
(a) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical myristic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical myristic acid;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of myristic acid, or a composition comprising myristic acid in said non-human organism or in the culture medium surrounding said non-human organism.

Accordingly, the present invention relates to a process for the production of nervonic acid, which comprises
(a) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical nervonic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical nervonic acid;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of nervonic acid, or a composition comprising nervonic acid in said non-human organism or in the culture medium surrounding said non-human organism.

Accordingly, the present invention relates to a process for the production of oleic acid, which comprises
(a) increasing or generating the activity of a polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, whereby the respective line discloses in column 7 the fine chemical oleic acid; or
increasing or generating the activity of an expression product of one or more nucleic acid molecule(s) comprising a polynucleotide as depicted in the respective line in column 5 or 8 of Table I, application no. 12, preferably the coding region thereof, or a homolog or a fragment thereof, whereby the respective line discloses in column 7 the fine chemical oleic acid;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of oleic acid, or a composition comprising oleic acid in said non-human organism or in the culture medium surrounding said non-human organism.

In a further embodiment the activity of the polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, is increased or generated non-targeted in the above-mentioned process in a microorganism or plant or a part thereof.

In a further embodiment said polypeptide has the activity of the respective polypeptide represented by a protein comprising a polypeptide as depicted in the respective line in column 5 of Table II, application no. 12.

In a further embodiment the activity of the expression product of one or more nucleic acid molecule(s) comprising a polynucleotide as depicted in the respective line in column 5 or 8 of Table I, application no. 12, preferably the coding region thereof, or a homolog or a fragment thereof, is increased or generated non-targeted in the above-mentioned process in a microorganism or plant or a part thereof.

In a further embodiment the activity of the polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, is increased or generated in the above-mentioned process in an organelle preferably in plastids or mitochondria, especially in plastids, of a microorganism or plant.

In a further embodiment said polypeptide has the activity of the respective polypeptide represented by a protein comprising a polypeptide as depicted in the respective line in column 5 of Table II, application no. 12.

In a further embodiment the activity of the expression product of one or more nucleic acid molecule(s) comprising a polynucleotide as depicted in the respective line in column 5 or 8 of Table I, application no. 12, preferably the coding region thereof, or a homolog or a fragment thereof, is increased or generated in the above-mentioned process in an organelle, preferably in plasids or mitochondria, especially in plastids, of a microorganism or plant.

In a further embodiment the activity of the polypeptide comprising a polypeptide as depicted in the respective line in column 5 or 8 of Table II, application no. 12, or a homolog or a fragment thereof, a consensus sequence or at least one polypeptide motif as depicted in the respective line in column 8 of Table IV, application no. 12, is increased or generated in the above-mentioned process in the cyctosol of a cell, of a microorganism or plant.

In a further embodiment said polypeptide has the activity of the respective polypeptide represented by a protein comprising a polypeptide as depicted in the respective line in column 5 of Table II, application no. 12.

In a further embodiment the activity of the expression product of one or more nucleic acid molecule(s) comprising a polynucleotide as depicted in the respective line in column 5 or 8 of Table I, application no. 12, preferably the coding region thereof, or a homolog or a fragment thereof, is increased or generated in the above-mentioned process in the cytosol of a cell, of a microorganism or plant.

[0039.1.8.12] to [0066.1.8.12] for the disclosure of these paragraphs see [0039.1.8.8] to [0066.1.8.8] above.

As mentioned above the nucleic acid sequences coding for the proteins as shown in Table II, application no. 12, column 5 or 8, or homologs or fragments thereof, are joined to a nucleic acid sequence encoding a plastidic transit peptide, e.g, if for the nucleic acid molecule in Table I, column 6 the term “plastidic” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in Table II, application no. 12, column 5 or 8, or homologs or fragments thereof, e.g., if for the nucleic acid molecule in Table I, column 6 the term “plastidic” is indicated.

[0068.1.8.12] to [0072.1.8.12] for the disclosure of these paragraphs see [0068.1.8.8] to [0072.1.8.8] above.

As mentioned above the nucleic acid sequences coding for the proteins as shown in the respective line in Table II, application no. 12, column 5 or 8, or homologs or fragments thereof, are joined to a nucleic acid sequence encoding a mitochondric transit peptide, e.g., if for the respective nucleic acid molecule in Table I, column 6 the term “mitochondrial” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in the respective line in Table II, application no. 12, column 5 or 8, or homologs or fragments thereof, e.g., if for the nucleic acid molecule in the respective line in Table I, column 6 the term “mitochondrial” is indicated. (For th eavoidance ofdoubt, th eterm “mitochondric” and “mitochondrial” are interchangeable.)

[0074.1.8.12] to [0075.1.8.12] for the disclosure of these paragraphs see [0074.1.8.8] to 0075.1.8.8] above.

Nucleic acid sequences coding for the transit peptides may be chemically synthesized either in part or wholly according to structure of transit peptide sequences disclosed in the prior art. Said natural or chemically synthesized sequences can be directly linked to the sequences encoding the mature protein or via a linker nucleic acid sequence, which may be typically less than 500 base pairs, preferably less than 450, 400, 350, 300, 250 or 200 base pairs, more preferably less than 150, 100, 90, 80, 70, 60, 50, 40 or 30 base pairs and most preferably less than 25, 20, 15, 12, 9, 6 or 3 base pairs in length and are in frame to the coding sequence. Furthermore favorable nucleic acid sequences encoding transit peptides may comprise sequences derived from more than one biological and/or chemical source and may include a nucleic acid sequence derived from the amino-terminal region of the mature protein, which in its native state is linked to the transit peptide. In a preferred embodiment of the invention said amino-terminal region of the mature protein is typically less than 150 amino acids, preferably less than 140, 130, 120, 110, 100 or 90 amino acids, more preferably less than 80, 70, 60, 50, 40, 35, 30, 25 or 20 amino acids and most preferably less than 19, 18, 17, 16, 15, 14, 13, 12, 11 or 10 amino acids in length. But even shorter or longer stretches are also possible. In addition target sequences, which facilitate the transport of proteins to other cell compartments such as the vacuole, endoplasmic reticulum, golgi complex, glyoxysomes, peroxisomes, mitochondria (in case of a plastidal transit peptide) or plastids, especially chloroplasts (in case of a mitochondric transit peptide) may be also part of the inventive nucleic acid sequence. The proteins translated from said inventive nucleic acid sequences are a kind of fusion proteins that means the nucleic acid sequences encoding the plastidal transit peptide for example the ones shown in Table a, preferably the last one of the Table a are joint to the respective nucleic acid sequences shown in Table I, application no. 12, columns 5 or 8, especially the coding region thereof, or homologs or fragments thereof, in case of nucleic acid sequences being expressed in plastids, and that means the nucleic acid sequences encoding the mitochondric transit peptide for example the ones shown in Table b, preferably the last one of the Table b are joint to the respective nucleic acid sequences shown in Table I, application no. 12, columns 5 or 8, especially the cod ing region thereof, or homologs or fragments thereof, in case of nucleic acid sequences being expressed in mitochondria. The person skilled in the art is able to join said sequences in a functional manner. Advantageously the transit peptide part is cleaved off from the protein part shown in Table II, application no. 12, columns 5 or 8, or a homolog or fragment thereof, during or after the transport into the organelles, preferably into the plastids or mitochondria, respectively.

All products of the cleavage of the preferred transit peptide shown in the last line of Table a have preferably the N-terminal amino acid sequences QIA CSS or QIA EFQLTT in front of the start methionine of the respective protein mentioned in Table II, application no. 12, column 5 or 8. or homologs or fragments thereof. Other short amino acid sequences of an range of 1 to 20 amino acids preferable 2 to 15 amino acids, more preferable 3 to 10 amino acids most preferably 4 to 8 amino acids are also possible in front of the start methionine of the protein mentioned in Table II, application no. 12, columns 5 or 8, or homologs or fragments thereof. In case of the amino acid sequence QIA CSS the three amino acids in front of the start methionine are stemming from the LIC (=ligatation independent cloning) cassette. Said short amino acid sequence is preferred in the case of the expression of E. coli genes. In case of the amino acid sequence QIA EFQLTT the six amino acids in front of the start methionine are stemming from the LIC cassette. Said short amino acid sequence is preferred in the case of the expression of S. cerevisiae genes. The skilled worker knowns that other short sequences are also useful in the expression of the respective nucleic acid molecule comprising a polynucleotide as depicted in Table I, application no. 12, column 5 or 8, preferably the coding region thereof, or a homolog or a fragment thereof. Furthermore the skilled worker is aware of the fact that there is not a need for such short sequences in the expression of the genes.

[0077.1.8.12] to [0078.1.8.12] for the disclosure of these paragraphs see [0077.1.8.8] to [0078.1.8.8] above.

Alternatively to the targeting of the respective sequences shown in Table II, application no. 12, columns 5 or 8, or homologs or fragments thereof, preferably of sequences in general encoded in the nucleus with the aid of the targeting sequences mentioned for example in Table a alone, Table b alone, or in combination with other targeting sequences preferably into plastids or mitochondria, respectively, the nucleic acids of the invention can directly be introduced into the plastidal or mitochondrial genome. Therefore in a preferred embodiment the respective nucleic acid sequences shown in the respective line in Table I, application no. 12, columns 5 or 8, preferably the coding region thereof, or homologs or fragments thereof, are directly introduced and expressed in plastids e.g., if for the nucleic acid molecule in the respective line in Table I, column 6, the term “plastidic” is indicated, or are directly introduced and expressed in mitochondria e.g., if for the nucleic acid molecule in the respective line in Table I, column 6 the term “mitochondrial” is indicated.

The term “introduced” in the context of this specification shall mean the insertion of a nucleic acid sequence into the organism by means of a “transfection”, “transduction” or preferably by “transformation”.

A plastid, such as a chloroplast, or a mitochondrion, has been “transformed” by an exogenous (preferably foreign) nucleic acid sequence if a nucleic acid sequence has been introduced into the plastid or mitochondrion that means that this sequence has crossed the membrane or the membranes of the plastid or of the mitochondrion. The exogenous, preferably foreign, DNA may be integrated (covalently linked) into plastid or mitochondrial DNA making up the genome of the plastid or mitochondrion, or it may remain unintegrated (e.g., by including a chloroplast origin of replication). “Stably” integrated DNA sequences are those, which are inherited through plastid or mitochondrion replication, thereby transferring new plastids or mitochondria, with the features of the integrated DNA sequence to the progeny.

[0080.1.8.12] to [0083.1.8.12] for the disclosure of these paragraphs see [0080.1.8.8] to [0083.1.8.8] above.

For the process of the present invention it may be of great advantage that by transforming the plastids the intraspecies specific transgene flow is blocked, because a lot of species such as corn, cotton and rice have a strict maternal inheritance of plastids. By placing the respective genes specified in the respective line in Table I, application no. 12, columns 5 or 8, preferably the coding region, or homologs or fragments thereof, e.g. if for the nucleic acid molecule in the respective line in column 6 of Table I the term “plastidic” is indicated, in the plastids of plants, these genes will not be present in the pollen of said plants.

A further preferred embodiment of the invention relates to the use of so called “chloroplast localization sequences”, in which a first RNA sequence or molecule is capable of transporting or “chaperoning” a second RNA sequence, such as a RNA sequence transcribed from the sequences depicted in the respective line in Table I, application no. 12, columns 5 or 8, preferably the coding region thereof, or a homolog or a fragment thereof, or a sequence encoding a protein, as depicted in the respective line in Table II, application no. 12, columns 5 and 8, or a homolog or a fragment thereof, from an external environment inside a cell or outside a plastid into a chloroplast. In one embodiment the chloroplast localization signal is substantially similar or complementary to a complete or intact viroid sequence. The chloroplast localization signal may be encoded by a DNA sequence, which is transcribed into the chloroplast localization RNA.

The term “viroid” refers to a naturally occurring single stranded RNA molecule (Flores, C. R. Acad. Sci. III. 324 (10), 943 (2001)). Viroids usually contain about 200-500 nucleotides and generally exist as circular molecules. Examples of viroids that contain chloroplast localization signals include but are not limited to ASBVd, PLMVd, CChMVd and ELVd. The viroid sequence or a functional part of it can be fused to the sequences depicted in the respective line in Table I, application no. 12, columns 5 or 8, preferably the coding region thereof, or a homolog or a fragment thereof, or a sequence encoding a protein, as depicted in the respective line in Table II, application no. 12, columns 5 or 8, or a homolog or a fragment thereof, in such a manner that the viroid sequence transports a sequence transcribed from a sequence as depicted in the respective line in Table I, application no. 12 columns 5 or 8, preferably the coding region thereof, or a homolog or a fragment thereof, or a sequence encoding a protein as depicted in the respective line in Table II, application no. 12 columns 5 or 8, or a homolog or a fragment thereof, into the chloroplasts, e.g. if for the nucleic acid molecule in column 6 of Table I the term “plastidic” is indicated. A preferred embodiment uses a modified ASBVd (Navarro et al., Virology. 268 (1), 218 (2000)).

In a further specific embodiment the protein to be expressed in the plastids such as the proteins depicted in the respective line in Table II, application no. 12, columns 5 or 8, or a homolog or a fragment thereof, e.g. if for the nucleic acid molecule in the respective line in column 6 of Table I the term “plastidic” is indicated, are encoded by different nucleic acids. Such a method is disclosed in WO 2004/040973, which shall be incorporated by reference. WO 2004/040973 teaches a method, which relates to the translocation of an RNA corresponding to a gene or gene fragment into the chloroplast by means of a chloroplast localization sequence.

The genes, which should be expressed in the plant or plant cells, are split into nucleic acid fragments, which are introduced into different compartments in the plant e.g. the nucleus, the plastids and/or mitochondria. Additionally plant cells are described in which the chloroplast contains a ribozyme fused at one end to an RNA encoding a fragment of a protein used in the inventive process such that the ribozyme can trans-splice the translocated fusion RNA to the RNA encoding the gene fragment to form and as the case may be reunite the nucleic acid fragments to an intact mRNA encoding a functional protein for example as disclosed in the respective line in Table II, application no. 12, columns 5 or 8, or a homolog or a fragment thereof.

In a preferred embodiment of the invention the nucleic acid sequences as shown in the respective line in Table I, application no. 12, columns 5 or 8, preferably the coding region thereof, or homologs or fragments thereof, e.g. if for the nucleic acid molecule in the respective line in column 6 of Table I the term “plastidic” is indicated, used in the inventive process are transformed into plastids, which are metabolically active. Those plastids should preferably maintain at a high copy number in the plant or plant tissue of interest, most preferably the chloroplasts found in green plant tissues, such as leaves or cotyledons or in seeds.

For a good expression in the plastids the nucleic acid sequences as shown in the respective line in Table I, application no. 12, columns 5 or 8, preferably the coding region thereof, or homologs or fragments thereof, e.g. if for the nucleic acid molecule in the respective line in column 6 of Table I the term “plastidic” is indicated, are introduced into an expression cassette using preferably a promoter and terminater, which are active in plastids preferably a chloroplast promoter. Examples of such promoters include the psbA promoter from the gene from spinach or pea, the rbcL promoter, and the atpB promoter from corn.

[0089.1.8.12] to [0092.1.8.12] for the disclosure of these paragraphs see [0089.1.8.8] to [0092.1.8.8] above.

Advantageously the process for the production of the fine chemical 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, and/or oleic acid, respectively, leads to an enhanced production of the respective fine chemical. The terms “enhanced” or “increase” mean at least a 10%, 20%, 30%, 40% or 50%, preferably at least 60%, 70%, 80%, 90% or 100%, more preferably 150%, 200%, 300%, 400% or 500% higher production of the respective fine chemical 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, and/or oleic acid in comparison to the wild-type as defined above, e.g. that means in comparison to a non-human organism without the aforementioned modification of the activity of a protein as shown in the respective line in Table II, application no. 12, column 5 or 8, or a fragment or a homolog thereof. The modification of the activity of a protein as shown in the respective line in Table II, application no. 12, column 5 or 8, or a homolog or a fragment thereof, or their combination can be achieved by joining the protein to a respective transit peptide, e.g. if for the respective encoding nucleic acid molecule in column 6 of Table I the term “plastidic” or “mitochondrial” is indicated.

Surprisingly it was found, that the transgenic expression of a protein as shown in the respective line in Table II, application no. 12, column 5, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, conferred a production of or an increase in 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, and/or oleic acid, respectively, to the transgenic non-human organism as compared to a corresponding non-transformed wild type.

In one embodiment the transgenic expression of a protein as shown in the respective line in Table II, application no. 12, column 5 or 8, or a homolog or a fragment thereof, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, confers a production of or an increase in 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, and/or oleic acid,respectively, to the transgenic non-human organism as compared to a corresponding non-transformed wild type.

Accordingly, in one embodiment, an increase of cerebronic acid in a non-human organism, as compared to a corresponding non-transformed wild type non-human organism, is conferred in the process of the invention, if the activity of a polypeptide showing the activity of a chlorophyllase, or if the activity of the polypeptide At1g19670, preferably represented by SEQ ID NO. 113652, or a homolog or fragment thereof, or if the activity of a polypeptide encoded by a nucleic acid molecule comprising the nucleic acid SEQ ID NO. 113651, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 113651 or polypeptide SEQ ID NO. 113652, respectively, or a homolog or a fragment thereof, is increased or generated, or if the activity chlorophyllase is increased or generated in a non-human organism, like a microorganism or a plant cell, plant or part thereof, especially with non-targeted localization, whereby the respective line discloses in column 7 the fine chemical cerebronic acid. For example, an increase of the cerebronic acid of at least 1 percent, particularly in a range of 25 to 38-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a chlorophyllase, or if the activity of the polypeptide At1g19670, preferably represented by SEQ ID NO. 113652, or a homolog or fragment thereof, or if the activity of a polypeptide encoded by a nucleic acid molecule comprising the nucleic acid SEQ ID NO. 113651, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 113651 or polypeptide SEQ ID NO. 113652, respectively, or a homolog or a fragment thereof, is increased or generated, or if the activity chlorophyllase is increased or generated in a non-human organism, like a microorganism or a plant cell, plant or part thereof, especially with non-targeted localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 29 to 47-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a transcription factor, or if the activity of the polypeptide At1g68320, preferably represented by SEQ ID NO. 1062, or a homolog or fragment thereof, or if the activity of a polypeptide encoded by a nucleic acid molecule comprising the nucleic acid SEQ ID NO. 1061, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 1061 or polypeptide SEQ ID NO. 1062, respectively, or a homolog or a fragment thereof, is increased or generated, or if the activity transcription factor is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 13 to 69-percent is conferred as compared to a corresponding non-transformed wild type non-human organism.

Accordingly, in one embodiment, an increase of cerebronic acid in a non-human organism, as compared to a corresponding non-transformed wild type non-human organism, is conferred in the process of the invention, if the activity of a polypeptide showing the activity of a protein phosphatase, or if the activity of the polypeptide At1g72770, preferably represented by SEQ ID NO. 19420, or a homolog or fragment thereof, or if the activity of a polypeptide encoded by a nucleic acid molecule comprising the nucleic acid SEQ ID NO. 19419, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 19419 or polypeptide SEQ ID NO. 19420, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 the fine chemical cerebronic acid. For example, an increase of the cerebronic acid of at least 1 percent, particularly in a range of 32 to 62-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a protein phosphatase, or if the activity of the polypeptide At1g72770, preferably represented by SEQ ID NO. 19420, or a homolog or fragment thereof, or if the activity of a polypeptide encoded by a nucleic acid molecule comprising the nucleic acid SEQ ID NO. 19419, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 19419 or polypeptide SEQ ID NO. 19420, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 14 to 58-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a protein phosphatase, or if the activity of the polypeptide At1g72770, preferably represented by SEQ ID NO. 19420, or a homolog or fragment thereof, or if the activity of a polypeptide encoded by a nucleic acid molecule comprising the nucleic acid SEQ ID NO. 19419, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 19419 or polypeptide SEQ ID NO. 19420, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 the fine chemical 2-hydroxybehenic acid. For example, an increase of the 2-hydroxybehenic acid of at least 1 percent, particularly in a range of 36 to 74-percent is conferred as compared to a corresponding non-transformed wild type non-human organism.

Accordingly, in one embodiment, an increase of 2-hydroxypalmitic acid in a non-human organism, as compared to a corresponding non-transformed wild type non-human organism, is conferred in the process of the invention, if the activity of a polypeptide showing the activity of a At2g33510-protein, or if the activity of the polypeptide At2g33510, preferably represented by SEQ ID NO. 98523, or a homolog or fragment thereof, or if the activity of a polypeptide encoded by a nucleic acid molecule comprising the nucleic acid SEQ ID NO. 98522, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 98522 or polypeptide SEQ ID NO. 98523, respectively, or a homolog or a fragment thereof, is increased or generated, or if the activity At2g33510-protein is increased or generated in a non-human organism, like a microorganism or a plant cell, plant or part thereof, especially with non-targeted localization, whereby the respective line discloses in column 7 the fine chemical 2-hydroxypalmitic acid. For example, an increase of the 2-hydroxypalmitic acid of at least 1 percent, particularly in a range of 20 to 24-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a heat shock transcription factor, or if the activity of the polypeptide At3g02990, preferably represented by SEQ ID NO. 68658, or a homolog or fragment thereof, or if the activity of a polypeptide encoded by a nucleic acid molecule comprising the nucleic acid SEQ ID NO. 68657, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 68657 or polypeptide SEQ ID NO. 68658, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 23 to 71-percent is conferred as compared to a corresponding non-transformed wild type non-human organism.

Accordingly, in one embodiment, an increase of cerebronic acid in a non-human organism, as compared to a corresponding non-transformed wild type non-human organism, is conferred in the process of the invention, if the activity of a polypeptide showing the activity of a adenosine kinase, or if the activity of the polypeptide At3g09820, preferably represented by SEQ ID NO. 1697, or a homolog or fragment thereof, or if the activity of a polypeptide encoded by a nucleic acid molecule comprising the nucleic acid SEQ ID NO. 1696, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 1696 or polypeptide SEQ ID NO. 1697, respectively, or a homolog or a fragment thereof, is increased or generated, or if the activity adenosine kinase is increased or generated in a non-human organism, like a microorganism or a plant cell, plant or part thereof, especially with non-targeted localization, whereby the respective line discloses in column 7 the fine chemical cerebronic acid. For example, an increase of the cerebronic acid of at least 1 percent, particularly in a range of 26 to 45-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a adenosine kinase, or if the activity of the polypeptide At3g09820, preferably represented by SEQ ID NO. 1697, or a homolog or fragment thereof, or if the activity of a polypeptide encoded by a nucleic acid molecule comprising the nucleic acid SEQ ID NO. 1696, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 1696 or polypeptide SEQ ID NO. 1697, respectively, or a homolog or a fragment thereof, is increased or generated, or if the activity adenosine kinase is increased or generated in a non-human organism, like a microorganism or a plant cell, plant or part thereof, especially with non-targeted localization, whereby the respective line discloses in column 7 the fine chemical 2-hydroxybehenic acid. For example, an increase of the 2-hydroxybehenic acid of at least 1 percent, particularly in a range of 25 to 52-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a DNA mismatch repair protein, or if the activity of the polypeptide At3g18524, preferably represented by SEQ ID NO. 22833, or a homolog or fragment thereof, or if the activity of a polypeptide encoded by a nucleic acid molecule comprising the nucleic acid SEQ ID NO. 22832, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 22832 or polypeptide SEQ ID NO. 22833, respectively, or a homolog or a fragment thereof, is increased or generated, or if the activity DNA mismatch repair protein is increased or generated in a non-human organism, like a microorganism or a plant cell, plant or part thereof, especially with non-targeted localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 25 to 36-percent is conferred as compared to a corresponding non-transformed wild type non-human organism.

Accordingly, in one embodiment, an increase of cerebronic acid in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 the fine chemical cerebronic acid. For example, an increase of the cerebronic acid of at least 1 percent, particularly in a range of 25 to 61-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 17 to 33-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 the fine chemical 2-hydroxybehenic acid. For example, an increase of the 2-hydroxybehenic acid of at least 1 percent, particularly in a range of 29 to 57-percent is conferred as compared to a corresponding non-transformed wild type non-human organism.

Accordingly, in one embodiment, an increase of 2-hydroxypalmitic acid in a non-human organism, as compared to a corresponding non-transformed wild type non-human organism, is conferred in the process of the invention, if the activity of a polypeptide showing the activity of a transcription factor, or if the activity of the polypeptide At4g09960, preferably represented by SEQ ID NO. 109718, or a homolog or fragment thereof, or if the activity of a polypeptide encoded by a nucleic acid molecule comprising the nucleic acid SEQ ID NO. 109717, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 109717 or polypeptide SEQ ID NO. 109718, respectively, or a homolog or a fragment thereof, is increased or generated, or if the activity transcription factor is increased or generated in a non-human organism, like a microorganism or a plant cell, plant or part thereof, especially with non-targeted localization, whereby the respective line discloses in column 7 the fine chemical 2-hydroxypalmitic acid. For example, an increase of the 2-hydroxypalmitic acid of at least 1 percent, particularly in a range of 22 to 50-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line discloses in column 7 the fine chemical 2-hydroxybehenic acid. For example, an increase of the 2-hydroxybehenic acid of at least 1 percent, particularly in a range of 28 to 59-percent is conferred as compared to a corresponding non-transformed wild type non-human organism.

Accordingly, in one embodiment, an increase of 2-hydroxypalmitic acid in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line discloses in column 7 the fine chemical 2-hydroxypalmitic acid. For example, an increase of the 2-hydroxypalmitic acid of at least 1 percent, particularly in a range of 23 to 52-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a monothiol glutaredoxin, or if the activity of the polypeptide At4g15670, preferably represented by SEQ ID NO. 3280, or a homolog or fragment thereof, or if the activity of a polypeptide encoded by a nucleic acid molecule comprising the nucleic acid SEQ ID NO. 3279, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 3279 or polypeptide SEQ ID NO. 3280, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 26 to 39-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a monothiol glutaredoxin, or if the activity of the polypeptide At4g15670, preferably represented by SEQ ID NO. 3280, or a homolog or fragment thereof, or if the activity of a polypeptide encoded by a nucleic acid molecule comprising the nucleic acid SEQ ID NO. 3279, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 3279 or polypeptide SEQ ID NO. 3280, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line discloses in column 7 the fine chemical 2-hydroxybehenic acid. For example, an increase of the 2-hydroxybehenic acid of at least 1 percent, particularly in a range of 24 to 104-percent is conferred as compared to a corresponding non-transformed wild type non-human organism.

Accordingly, in one embodiment, an increase of cerebronic acid in a non-human organism, as compared to a corresponding non-transformed wild type non-human organism, is conferred in the process of the invention, if the activity of a polypeptide showing the activity of a monothiol glutaredoxin, or if the activity of the polypeptide At4g15690, preferably represented by SEQ ID NO. 23845, or a homolog or fragment thereof, or if the activity of a polypeptide encoded by a nucleic acid molecule comprising the nucleic acid SEQ ID NO. 23844, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 23844 or polypeptide SEQ ID NO. 23845, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line discloses in column 7 the fine chemical cerebronic acid. For example, an increase of the cerebronic acid of at least 1 percent, particularly in a range of 23 to 48-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a protein phosphatase, or if the activity of the polypeptide At4g26080, preferably represented by SEQ ID NO. 68850, or a homolog or fragment thereof, or if the activity of a polypeptide encoded by a nucleic acid molecule comprising the nucleic acid SEQ ID NO. 68849, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 68849 or polypeptide SEQ ID NO. 68850, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 the fine chemical 2-hydroxybehenic acid. For example, an increase of the 2-hydroxybehenic acid of at least 1 percent, particularly in a range of 26 to 48-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a protein phosphatase, or if the activity of the polypeptide At4g26080, preferably represented by SEQ ID NO. 68850, or a homolog or fragment thereof, or if the activity of a polypeptide encoded by a nucleic acid molecule comprising the nucleic acid SEQ ID NO. 68849, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 68849 or polypeptide SEQ ID NO. 68850, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 21 to 43-percent is conferred as compared to a corresponding non-transformed wild type non-human organism.

Accordingly, in one embodiment, an increase of cerebronic acid in a non-human organism, as compared to a corresponding non-transformed wild type non-human organism, is conferred in the process of the invention, if the activity of a polypeptide showing the activity of a glutaredoxin, or if the activity of the polypeptide At4g33040, preferably represented by SEQ ID NO. 4103, or a homolog or fragment thereof, or if the activity of a polypeptide encoded by a nucleic acid molecule comprising the nucleic acid SEQ ID NO. 4102, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 4102 or polypeptide SEQ ID NO. 4103, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 the fine chemical cerebronic acid. For example, an increase of the cerebronic acid of at least 1 percent, particularly in a range of 25 to 58-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a glutaredoxin, or if the activity of the polypeptide At4g33040, preferably represented by SEQ ID NO. 4103, or a homolog or fragment thereof, or if the activity of a polypeptide encoded by a nucleic acid molecule comprising the nucleic acid SEQ ID NO. 4102, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 4102 or polypeptide SEQ ID NO. 4103, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 the fine chemical hexadecadienoic acid. For example, an increase of the hexadecadienoic acid of at least 1 percent, particularly in a range of 20 to 61-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a glutaredoxin, or if the activity of the polypeptide At4g33040, preferably represented by SEQ ID NO. 4103, or a homolog or fragment thereof, or if the activity of a polypeptide encoded by a nucleic acid molecule comprising the nucleic acid SEQ ID NO. 4102, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 4102 or polypeptide SEQ ID NO. 4103, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 13 to 35-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line discloses in column 7 the fine chemical 2-hydroxybehenic acid. For example, an increase of the 2-hydroxybehenic acid of at least 1 percent, particularly in a range of 32 to 125-percent is conferred as compared to a corresponding non-transformed wild type non-human organism.

Accordingly, in one embodiment, an increase of 2-hydroxypalmitic acid in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line discloses in column 7 the fine chemical 2-hydroxypalmitic acid. For example, an increase of the 2-hydroxypalmitic acid of at least 1 percent, particularly in a range of 18 to 97-percent is conferred as compared to a corresponding non-transformed wild type non-human organism.

Accordingly, in one embodiment, an increase of oleic acid in a non-human organism, as compared to a corresponding non-transformed wild type non-human organism, is conferred in the process of the invention, if the activity of a polypeptide showing the activity of a acyldesaturase, or if the activity of the polypeptide At5g16230, preferably represented by SEQ ID NO. 122566, or a homolog or fragment thereof, or if the activity of a polypeptide encoded by a nucleic acid molecule comprising the nucleic acid SEQ ID NO. 122565, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 122565 or polypeptide SEQ ID NO. 122566, respectively, or a homolog or a fragment thereof, is increased or generated, or if the activity acyl-desaturase is increased or generated in a non-human organism, like a microorganism or a plant cell, plant or part thereof, especially with non-targeted localization, whereby the respective line discloses in column 7 the fine chemical oleic acid. For example, an increase of the oleic acid of at least 1 percent, particularly in a range of 137 to 225-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a monothiol glutaredoxin, or if the activity of the polypeptide At5g18600, preferably represented by SEQ ID NO. 4905, or a homolog or fragment thereof, or if the activity of a polypeptide encoded by a nucleic acid molecule comprising the nucleic acid SEQ ID NO. 4904, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 4904 or polypeptide SEQ ID NO. 4905, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 17 to 37-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a protein phosphatase, or if the activity of the polypeptide At5g57050, preferably represented by SEQ ID NO. 5319, or a homolog or fragment thereof, or if the activity of a polypeptide encoded by a nucleic acid molecule comprising the nucleic acid SEQ ID NO. 5318, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 5318 or polypeptide SEQ ID NO. 5319, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 15 to 33-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a protein kinase, or if the activity of the polypeptide At5g66710, preferably represented by SEQ ID NO. 122804, or a homolog or fragment thereof, or if the activity of a polypeptide encoded by a nucleic acid molecule comprising the nucleic acid SEQ ID NO. 122803, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 122803 or polypeptide SEQ ID NO. 122804, respectively, or a homolog or a fragment thereof, is increased or generated, or if the activity protein kinase is increased or generated in a non-human organism, like a microorganism or a plant cell, plant or part thereof, especially with non-targeted localization, whereby the respective line discloses in column 7 the fine chemical hexadecadienoic acid. For example, an increase of the hexadecadienoic acid of at least 1 percent, particularly in a range of 29 to 120-percent is conferred as compared to a corresponding non-transformed wild type non-human organism.

Accordingly, in one embodiment, an increase of cerebronic acid in a non-human organism, as compared to a corresponding non-transformed wild type non-human organism, is conferred in the process of the invention, if the activity of a polypeptide showing the activity of a CDP-diacylglycerol-serine O-phosphatidyltransferase, or if the activity of the polypeptide AvinDRAFT_{—}0035, preferably represented by SEQ ID NO. 70584, or a homolog or fragment thereof, or if the activity of a polypeptide encoded by a nucleic acid molecule comprising the nucleic acid SEQ ID NO. 70583, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 70583 or polypeptide SEQ ID NO. 70584, respectively, or a homolog or a fragment thereof, is increased or generated, or if the activity CDP-diacylglycerolserine O-phosphatidyltransferase is increased or generated in a non-human organism, like a microorganism or a plant cell, plant or part thereof, especially with non-targeted localization, whereby the respective line discloses in column 7 the fine chemical cerebronic acid. For example, an increase of the cerebronic acid of at least 1 percent, particularly in a range of 22 to 38-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a CDP-diacylglycerol-serine O-phosphatidyltransferase, or if the activity of the polypeptide AvinDRAFT_{—}0035, preferably represented by SEQ ID NO. 70584, or a homolog or fragment thereof, or if the activity of a polypeptide encoded by a nucleic acid molecule comprising the nucleic acid SEQ ID NO. 70583, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 70583 or polypeptide SEQ ID NO. 70584, respectively, or a homolog or a fragment thereof, is increased or generated, or if the activity CDP-diacylglycerolserine O-phosphatidyltransferase is increased or generated in a non-human organism, like a microorganism or a plant cell, plant or part thereof, especially with non-targeted localization, whereby the respective line discloses in column 7 the fine chemical 2-hydroxybehenic acid. For example, an increase of the 2-hydroxybehenic acid of at least 1 percent, particularly in a range of 29 to 56-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a CDP-diacylglycerol-serine O-phosphatidyltransferase, or if the activity of the polypeptide AvinDRAFT_{—}0035, preferably represented by SEQ ID NO. 70584, or a homolog or fragment thereof, or if the activity of a polypeptide encoded by a nucleic acid molecule comprising the nucleic acid SEQ ID NO. 70583, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 70583 or polypeptide SEQ ID NO. 70584, respectively, or a homolog or a fragment thereof, is increased or generated, or if the activity CDP-diacylglycerol-serine O-phosphatidyltransferase is increased or generated in a non-human organism, like a microorganism or a plant cell, plant or part thereof, especially with non-targeted localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 28 to 48-percent is conferred as compared to a corresponding non-transformed wild type non-human organism.

Accordingly, in one embodiment, an increase of 2-hydroxypalmitic acid in a non-human organism, as compared to a corresponding non-transformed wild type non-human organism, is conferred in the process of the invention, if the activity of a polypeptide showing the activity of a CDP-diacylglycerol-serine O-phosphatidyltransferase, or if the activity of the polypeptide AvinDRAFT_{—}0035, preferably represented by SEQ ID NO. 70584, or a homolog or fragment thereof, or if the activity of a polypeptide encoded by a nucleic acid molecule comprising the nucleic acid SEQ ID NO. 70583, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 70583 or polypeptide SEQ ID NO. 70584, respectively, or a homolog or a fragment thereof, is increased or generated, or if the activity CDP-diacylglycerolserine O-phosphatidyltransferase is increased or generated in a non-human organism, like a microorganism or a plant cell, plant or part thereof, especially with non-targeted localization, whereby the respective line discloses in column 7 the fine chemical 2-hydroxypalmitic acid. For example, an increase of the 2-hydroxypalmitic acid of at least 1 percent, particularly in a range of 20 to 53-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a sec-independent protein translocase, or if the activity of the polypeptide AvinDRAFT_{—}1624, preferably represented by SEQ ID NO. 26197, or a homolog or fragment thereof, or if the activity of a polypeptide encoded by a nucleic acid molecule comprising the nucleic acid SEQ ID NO. 26196, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 26196 or polypeptide SEQ ID NO. 26197, respectively, or a homolog or a fragment thereof, is increased or generated, or if the activity sec-independent protein translocase is increased or generated in a non-human organism, like a microorganism or a plant cell, plant or part thereof, especially with non-targeted localization, whereby the respective line discloses in column 7 the fine chemical hexadecadienoic acid. For example, an increase of the hexadecadienoic acid of at least 1 percent, particularly in a range of 22 to 50-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a transcriptional regulator protein, or if the activity of the polypeptide AvinDRAFT_{—}2010, preferably represented by SEQ ID NO. 114199, or a homolog or fragment thereof, or if the activity of a polypeptide encoded by a nucleic acid molecule comprising the nucleic acid SEQ ID NO. 114198, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 114198 or polypeptide SEQ ID NO. 114199, respectively, or a homolog or a fragment thereof, is increased or generated, or if the activity transcriptional regulator protein is increased or generated in a non-human organism, like a microorganism or a plant cell, plant or part thereof, especially with non-targeted localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 24 to 38-percent is conferred as compared to a corresponding non-transformed wild type non-human organism.

Accordingly, in one embodiment, an increase of 2-hydroxypalmitic acid in a non-human organism, as compared to a corresponding non-transformed wild type non-human organism, is conferred in the process of the invention, if the activity of a polypeptide showing the activity of a beta-hydroxylase, or if the activity of the polypeptide AvinDRAFT_{—}2091, preferably represented by SEQ ID NO. 6041, or a homolog or fragment thereof, or if the activity of a polypeptide encoded by a nucleic acid molecule comprising the nucleic acid SEQ ID NO. 6040, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 6040 or polypeptide SEQ ID NO. 6041, respectively, or a homolog or a fragment thereof, is increased or generated, or if the activity beta-hydroxylase is increased or generated in a non-human organism, like a microorganism or a plant cell, plant or part thereof, especially with non-targeted localization, whereby the respective line discloses in column 7 the fine chemical 2-hydroxypalmitic acid. For example, an increase of the 2-hydroxypalmitic acid of at least 1 percent, particularly in a range of 24 to 44-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a 30S ribosomal protein S3, or if the activity of the polypeptide AvinDRAFT_{—}2365, preferably represented by SEQ ID NO. 71326, or a homolog or fragment thereof, or if the activity of a polypeptide encoded by a nucleic acid molecule comprising the nucleic acid SEQ ID NO. 71325, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 71325 or polypeptide SEQ ID NO. 71326, respectively, or a homolog or a fragment thereof, is increased or generated, or if the activity 30S ribosomal protein S3 is increased or generated in a non-human organism, like a microorganism or a plant cell, plant or part thereof, especially with non-targeted localization, whereby the respective line discloses in column 7 the fine chemical hexadecadienoic acid. For example, an increase of the hexadecadienoic acid of at least 1 percent, particularly in a range of 38 to 99-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a acyl-CoA synthase, or if the activity of the polypeptide AvinDRAFT_{—}2754, preferably represented by SEQ ID NO. 28041, or a homolog or fragment thereof, or if the activity of a polypeptide encoded by a nucleic acid molecule comprising the nucleic acid SEQ ID NO. 28040, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 28040 or polypeptide SEQ ID NO. 28041, respectively, or a homolog or a fragment thereof, is increased or generated, or if the activity acyl-CoA synthase is increased or generated in a non-human organism, like a microorganism or a plant cell, plant or part thereof, especially with non-targeted localization, whereby the respective line discloses in column 7 the fine chemical 2-hydroxybehenic acid. For example, an increase of the 2-hydroxybehenic acid of at least 1 percent, particularly in a range of 28 to 72-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a heat shock protein, or if the activity of the polypeptide AvinDRAFT_{—}2827, preferably represented by SEQ ID NO. 122949, or a homolog or fragment thereof, or if the activity of a polypeptide encoded by a nucleic acid molecule comprising the nucleic acid SEQ ID NO. 122948, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 122948 or polypeptide SEQ ID NO. 122949, respectively, or a homolog or a fragment thereof, is increased or generated, or if the activity heat shock protein is increased or generated in a non-human organism, like a microorganism or a plant cell, plant or part thereof, especially with non-targeted localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 23 to 80-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a oxidoreductase, or if the activity of the polypeptide AvinDRAFT_{—}3253, preferably represented by SEQ ID NO. 29501, or a homolog or fragment thereof, or if the activity of a polypeptide encoded by a nucleic acid molecule comprising the nucleic acid SEQ ID NO. 29500, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 29500 or polypeptide SEQ ID NO. 29501, respectively, or a homolog or a fragment thereof, is increased or generated, or if the activity oxidoreductase is increased or generated in a non-human organism, like a microorganism or a plant cell, plant or part thereof, especially with non-targeted localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 26 to 56-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a recombinase A, or if the activity of the polypeptide AvinDRAFT_{—}3629, preferably represented by SEQ ID NO. 114232, or a homolog or fragment thereof, or if the activity of a polypeptide encoded by a nucleic acid molecule comprising the nucleic acid SEQ ID NO. 114231, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 114231 or polypeptide SEQ ID NO. 114232, respectively, or a homolog or a fragment thereof, is increased or generated, or if the activity recombinase A is increased or generated in a non-human organism, like a microorganism or a plant cell, plant or part thereof, especially with non-targeted localization, whereby the respective line discloses in column 7 the fine chemical hexadecadienoic acid. For example, an increase of the hexadecadienoic acid of at least 1 percent, particularly in a range of 23 to 113-percent is conferred as compared to a corresponding non-transformed wild type non-human organism.

Accordingly, in one embodiment, an increase of lauric acid in a non-human organism, as compared to a corresponding non-transformed wild type non-human organism, is conferred in the process of the invention, if the activity of a polypeptide showing the activity of a ABC transporter component, or if the activity of the polypeptide AVINDRAFT_{—}4128, preferably represented by SEQ ID NO. 72521, or a homolog or fragment thereof, or if the activity of a polypeptide encoded by a nucleic acid molecule comprising the nucleic acid SEQ ID NO. 72520, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 72520 or polypeptide SEQ ID NO. 72521, respectively, or a homolog or a fragment thereof, is increased or generated, or if the activity ABC transporter component is increased or generated in a non-human organism, like a microorganism or a plant cell, plant or part thereof, especially with non-targeted localization, whereby the respective line discloses in column 7 the fine chemical lauric acid. For example, an increase of the lauric acid of at least 1 percent, particularly in a range of 44 to 99-percent is conferred as compared to a corresponding non-transformed wild type non-human organism.

Accordingly, in one embodiment, an increase of 2-hydroxypalmitic acid in a non-human organism, as compared to a corresponding non-transformed wild type non-human organism, is conferred in the process of the invention, if the activity of a polypeptide showing the activity of a hydrolase, or if the activity of the polypeptide AvinDRAFT_{—}5103, preferably represented by SEQ ID NO. 6511, or a homolog or fragment thereof, or if the activity of a polypeptide encoded by a nucleic acid molecule comprising the nucleic acid SEQ ID NO. 6510, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 6510 or polypeptide SEQ ID NO. 6511, respectively, or a homolog or a fragment thereof, is increased or generated, or if the activity hydrolase is increased or generated in a non-human organism, like a microorganism or a plant cell, plant or part thereof, especially with non-targeted localization, whereby the respective line discloses in column 7 the fine chemical 2-hydroxypalmitic acid. For example, an increase of the 2-hydroxypalmitic acid of at least 1 percent, particularly in a range of 20 to 76-percent is conferred as compared to a corresponding non-transformed wild type non-human organism.

Accordingly, in one embodiment, an increase of 2-hydroxypalmitic acid in a non-human organism, as compared to a corresponding non-transformed wild type non-human organism, is conferred in the process of the invention, if the activity of a polypeptide showing the activity of a glutamate-ammonia-ligase, or if the activity of the polypeptide AvinDRAFT_{—}5246, preferably represented by SEQ ID NO. 32038, or a homolog or fragment thereof, or if the activity of a polypeptide encoded by a nucleic acid molecule comprising the nucleic acid SEQ ID NO. 32037, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 32037 or polypeptide SEQ ID NO. 32038, respectively, or a homolog or a fragment thereof, is increased or generated, or if the activity glutamate-ammonia-ligase is increased or generated in a non-human organism, like a microorganism or a plant cell, plant or part thereof, especially with non-targeted localization, whereby the respective line discloses in column 7 the fine chemical 2-hydroxypalmitic acid. For example, an increase of the 2-hydroxypalmitic acid of at least 1 percent, particularly in a range of 21 to 40-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a fumarylacetoacetate hydrolase, or if the activity of the polypeptide AvinDRAFT_{—}5292, preferably represented by SEQ ID NO. 32309, or a homolog or fragment thereof, or if the activity of a polypeptide encoded by a nucleic acid molecule comprising the nucleic acid SEQ ID NO. 32308, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 32308 or polypeptide SEQ ID NO. 32309, respectively, or a homolog or a fragment thereof, is increased or generated, or if the activity fumarylacetoacetate hydrolase is increased or generated in a non-human organism, like a microorganism or a plant cell, plant or part thereof, especially with non-targeted localization, whereby the respective line discloses in column 7 the fine chemical 2-hydroxybehenic acid. For example, an increase of the 2-hydroxybehenic acid of at least 1 percent, particularly in a range of 64 to 74-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a N-acetyl-gamma-glutamyl-phosphate reductase, or if the activity of the polypeptide AvinDRAFT_{—}5466, preferably represented by SEQ ID NO. 123088, or a homolog or fragment thereof, or if the activity of a polypeptide encoded by a nucleic acid molecule comprising the nucleic acid SEQ ID NO. 123087, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 123087 or polypeptide SEQ ID NO. 123088, respectively, or a homolog or a fragment thereof, is increased or generated, or if the activity N-acetyl-gamma-glutamyl-phosphate reductase is increased or generated in a non-human organism, like a microorganism or a plant cell, plant or part thereof, especially with non-targeted localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 22 to 55-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a HesB/YadR/YfhF family protein, or if the activity of the polypeptide AvinDRAFT_{—}5467, preferably represented by SEQ ID NO. 32649, or a homolog or fragment thereof, or if the activity of a polypeptide encoded by a nucleic acid molecule comprising the nucleic acid SEQ ID NO. 32648, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 32648 or polypeptide SEQ ID NO. 32649, respectively, or a homolog or a fragment thereof, is increased or generated, or if the activity HesB/YadR/YfhF family protein is increased or generated in a non-human organism, like a microorganism or a plant cell, plant or part thereof, especially with non-targeted localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 22 to 32-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a purine nucleoside phosphorylase, or if the activity of the polypeptide AvinDRAFT_{—}6700, preferably represented by SEQ ID NO. 34045, or a homolog or fragment thereof, or if the activity of a polypeptide encoded by a nucleic acid molecule comprising the nucleic acid SEQ ID NO. 34044, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 34044 or polypeptide SEQ ID NO. 34045, respectively, or a homolog or a fragment thereof, is increased or generated, or if the activity purine nucleoside phosphorylase is increased or generated in a non-human organism, like a microorganism or a plant cell, plant or part thereof, especially with non-targeted localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 25 to 48-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a b0050-protein, or if the activity of the polypeptide B0050, preferably represented by SEQ ID NO. 103959, or a homolog or fragment thereof, or if the activity of a polypeptide encoded by a nucleic acid molecule comprising the nucleic acid SEQ ID NO. 103958, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 103958 or polypeptide SEQ ID NO. 103959, respectively, or a homolog or a fragment thereof, is increased or generated, or if the activity b0050-protein is increased or generated in a non-human organism, like a microorganism or a plant cell, plant or part thereof, especially with non-targeted localization, whereby the respective line discloses in column 7 the fine chemical 2-hydroxybehenic acid. For example, an increase of the 2-hydroxybehenic acid of at least 1 percent, particularly in a range of 33 to 55-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a b0050-protein, or if the activity of the polypeptide B0050, preferably represented by SEQ ID NO. 103959, or a homolog or fragment thereof, or if the activity of a polypeptide encoded by a nucleic acid molecule comprising the nucleic acid SEQ ID NO. 103958, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 103958 or polypeptide SEQ ID NO. 103959, respectively, or a homolog or a fragment thereof, is increased or generated, or if the activity b0050-protein is increased or generated in a non-human organism, like a microorganism or a plant cell, plant or part thereof, especially with non-targeted localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 26 to 38-percent is conferred as compared to a corresponding non-transformed wild type non-human organism.

Accordingly, in one embodiment, an increase of cerebronic acid in a non-human organism, as compared to a corresponding non-transformed wild type non-human organism, is conferred in the process of the invention, if the activity of a polypeptide showing the activity of a b0249-protein, or if the activity of the polypeptide B0249, preferably represented by SEQ ID NO. 123474, or a homolog or fragment thereof, or if the activity of a polypeptide encoded by a nucleic acid molecule comprising the nucleic acid SEQ ID NO. 123473, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 123473 or polypeptide SEQ ID NO. 123474, respectively, or a homolog or a fragment thereof, is increased or generated, or if the activity b0249-protein is increased or generated in a non-human organism, like a microorganism or a plant cell, plant or part thereof, especially with non-targeted localization, whereby the respective line discloses in column 7 the fine chemical cerebronic acid. For example, an increase of the cerebronic acid of at least 1 percent, particularly in a range of 27 to 48-percent is conferred as compared to a corresponding non-transformed wild type non-human organism.

Accordingly, in one embodiment, an increase of behenic acid in a non-human organism, as compared to a corresponding non-transformed wild type non-human organism, is conferred in the process of the invention, if the activity of a polypeptide showing the activity of a betaine aldehyde dehydrogenase, or if the activity of the polypeptide B0312, preferably represented by SEQ ID NO. 121112, or a homolog or fragment thereof, or if the activity of a polypeptide encoded by a nucleic acid molecule comprising the nucleic acid SEQ ID NO. 121111, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 121111 or polypeptide SEQ ID NO. 121112, respectively, or a homolog or a fragment thereof, is increased or generated, or if the activity betaine aldehyde dehydrogenase is increased or generated in a non-human organism, like a microorganism or a plant cell, plant or part thereof, especially with non-targeted localization, whereby the respective line discloses in column 7 the fine chemical behenic acid. For example, an increase of the behenic acid of at least 1 percent, particularly in a range of 11 to 14-percent is conferred as compared to a corresponding non-transformed wild type non-human organism.

Accordingly, in one embodiment, an increase of eicosanoic acid in a non-human organism, as compared to a corresponding non-transformed wild type non-human organism, is conferred in the process of the invention, if the activity of a polypeptide showing the activity of a betaine aldehyde dehydrogenase, or if the activity of the polypeptide B0312, preferably represented by SEQ ID NO. 121112, or a homolog or fragment thereof, or if the activity of a polypeptide encoded by a nucleic acid molecule comprising the nucleic acid SEQ ID NO. 121111, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 121111 or polypeptide SEQ ID NO. 121112, respectively, or a homolog or a fragment thereof, is increased or generated, or if the activity betaine aldehyde dehydrogenase is increased or generated in a non-human organism, like a microorganism or a plant cell, plant or part thereof, especially with non-targeted localization, whereby the respective line discloses in column 7 the fine chemical eicosanoic acid. For example, an increase of the eicosanoic acid of at least 1 percent, particularly in a range of 11 to 14-percent is conferred as compared to a corresponding non-transformed wild type non-human organism.

Accordingly, in one embodiment, an increase of myristic acid in a non-human organism, as compared to a corresponding non-transformed wild type non-human organism, is conferred in the process of the invention, if the activity of a polypeptide showing the activity of a 2,3-dihydroxyphenylpropionate 1,2-dioxygenase, or if the activity of the polypeptide B0348, preferably represented by SEQ ID NO. 7270, or a homolog or fragment thereof, or if the activity of a polypeptide encoded by a nucleic acid molecule comprising the nucleic acid SEQ ID NO. 7269, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 7269 or polypeptide SEQ ID NO. 7270, respectively, or a homolog or a fragment thereof, is increased or generated, or if the activity 2,3-dihydroxyphenylpropionate 1,2-dioxygenase is increased or generated in a non-human organism, like a microorganism or a plant cell, plant or part thereof, especially with plastidic localization, whereby the respective line discloses in column 7 the fine chemical myristic acid. For example, an increase of the myristic acid of at least 1 percent, particularly in a range of 21 to 39-percent is conferred as compared to a corresponding non-transformed wild type non-human organism.

Accordingly, in one embodiment, an increase of oleic acid in a non-human organism, as compared to a corresponding non-transformed wild type non-human organism, is conferred in the process of the invention, if the activity of a polypeptide showing the activity of a 2,3-dihydroxyphenylpropionate 1,2-dioxygenase, or if the activity of the polypeptide B0348, preferably represented by SEQ ID NO. 7270, or a homolog or fragment thereof, or if the activity of a polypeptide encoded by a nucleic acid molecule comprising the nucleic acid SEQ ID NO. 7269, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 7269 or polypeptide SEQ ID NO. 7270, respectively, or a homolog or a fragment thereof, is increased or generated, or if the activity 2,3-dihydroxyphenylpropionate 1,2-dioxygenase is increased or generated in a non-human organism, like a microorganism or a plant cell, plant or part thereof, especially with plastidic localization, whereby the respective line discloses in column 7 the fine chemical oleic acid. For example, an increase of the oleic acid of at least 1 percent, particularly in a range of 24 to 43-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a b0371-protein, or if the activity of the polypeptide B0371, preferably represented by SEQ ID NO. 121638, or a homolog or fragment thereof, or if the activity of a polypeptide encoded by a nucleic acid molecule comprising the nucleic acid SEQ ID NO. 121637, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 121637 or polypeptide SEQ ID NO. 121638, respectively, or a homolog or a fragment thereof, is increased or generated, or if the activity b0371-protein is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 17 to 32-percent is conferred as compared to a corresponding non-transformed wild type non-human organism.

Accordingly, in one embodiment, an increase of cerebronic acid in a non-human organism, as compared to a corresponding non-transformed wild type non-human organism, is conferred in the process of the invention, if the activity of a polypeptide showing the activity of a ATP-binding component of a transport system, or if the activity of the polypeptide B0449, preferably represented by SEQ ID NO. 7334, or a homolog or fragment thereof, or if the activity of a polypeptide encoded by a nucleic acid molecule comprising the nucleic acid SEQ ID NO. 7333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 7333 or polypeptide SEQ ID NO. 7334, respectively, or a homolog or a fragment thereof, is increased or generated, or if the activity ATP-binding component of a transport system is increased or generated in a non-human organism, like a microorganism or a plant cell, plant or part thereof, especially with non-targeted localization, whereby the respective line discloses in column 7 the fine chemical cerebronic acid. For example, an increase of the cerebronic acid of at least 1 percent, particularly in a range of 24 to 205-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a ATP-binding component of a transport system, or if the activity of the polypeptide B0449, preferably represented by SEQ ID NO. 7334, or a homolog or fragment thereof, or if the activity of a polypeptide encoded by a nucleic acid molecule comprising the nucleic acid SEQ ID NO. 7333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 7333 or polypeptide SEQ ID NO. 7334, respectively, or a homolog or a fragment thereof, is increased or generated, or if the activity ATP-binding component of a transport system is increased or generated in a non-human organism, like a microorganism or a plant cell, plant or part thereof, especially with non-targeted localization, whereby the respective line discloses in column 7 the fine chemical 2-hydroxybehenic acid. For example, an increase of the 2-hydroxybehenic acid of at least 1 percent, particularly in a range of 28 to 193-percent is conferred as compared to a corresponding non-transformed wild type non-human organism.

Accordingly, in one embodiment, an increase of 2-hydroxypalmitic acid in a non-human organism, as compared to a corresponding non-transformed wild type non-human organism, is conferred in the process of the invention, if the activity of a polypeptide showing the activity of a ATP-binding component of a transport system, or if the activity of the polypeptide B0449, preferably represented by SEQ ID NO. 7334, or a homolog or fragment thereof, or if the activity of a polypeptide encoded by a nucleic acid molecule comprising the nucleic acid SEQ ID NO. 7333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 7333 or polypeptide SEQ ID NO. 7334, respectively, or a homolog or a fragment thereof, is increased or generated, or if the activity ATP-binding component of a transport system is increased or generated in a non-human organism, like a microorganism or a plant cell, plant or part thereof, especially with non-targeted localization, whereby the respective line discloses in column 7 the fine chemical 2-hydroxypalmitic acid. For example, an increase of the 2-hydroxypalmitic acid of at least 1 percent, particularly in a range of 19 to 207-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a b0518-protein, or if the activity of the polypeptide B0518, preferably represented by SEQ ID NO. 35937, or a homolog or fragment thereof, or if the activity of a polypeptide encoded by a nucleic acid molecule comprising the nucleic acid SEQ ID NO. 35936, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 35936 or polypeptide SEQ ID NO. 35937, respectively, or a homolog or a fragment thereof, is increased or generated, or if the activity b0518-protein is increased or generated in a non-human organism, like a microorganism or a plant cell, plant or part thereof, especially with non-targeted localization, whereby the respective line discloses in column 7 the fine chemical hexadecadienoic acid. For example, an increase of the hexadecadienoic acid of at least 1 percent, particularly in a range of 25 to 64-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a sensor histidine kinase, or if the activity of the polypeptide B0695, preferably represented by SEQ ID NO. 93679, or a homolog or fragment thereof, or if the activity of a polypeptide encoded by a nucleic acid molecule comprising the nucleic acid SEQ ID NO. 93678, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 93678 or polypeptide SEQ ID NO. 93679, respectively, or a homolog or a fragment thereof, is increased or generated, or if the activity sensor histidine kinase is increased or generated in a non-human organism, like a microorganism or a plant cell, plant or part thereof, especially with non-targeted localization, whereby the respective line discloses in column 7 the fine chemical 2-hydroxybehenic acid. For example, an increase of the 2-hydroxybehenic acid of at least 1 percent, particularly in a range of 38 to 63-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a sensor histidine kinase, or if the activity of the polypeptide B0695, preferably represented by SEQ ID NO. 93679, or a homolog or fragment thereof, or if the activity of a polypeptide encoded by a nucleic acid molecule comprising the nucleic acid SEQ ID NO. 93678, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 93678 or polypeptide SEQ ID NO. 93679, respectively, or a homolog or a fragment thereof, is increased or generated, or if the activity sensor histidine kinase is increased or generated in a non-human organism, like a microorganism or a plant cell, plant or part thereof, especially with non-targeted localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 24 to 82-percent is conferred as compared to a corresponding non-transformed wild type non-human organism.

Accordingly, in one embodiment, an increase of cerebronic acid in a non-human organism, as compared to a corresponding non-transformed wild type non-human organism, is conferred in the process of the invention, if the activity of a polypeptide showing the activity of a citrate synthase, or if the activity of the polypeptide B0720, preferably represented by SEQ ID NO. 123499, or a homolog or fragment thereof, or if the activity of a polypeptide encoded by a nucleic acid molecule comprising the nucleic acid SEQ ID NO. 123498, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 123498 or polypeptide SEQ ID NO. 123499, respectively, or a homolog or a fragment thereof, is increased or generated, or if the activity citrate synthase is increased or generated in a non-human organism, like a microorganism or a plant cell, plant or part thereof, especially with plastidic localization, whereby the respective line discloses in column 7 the fine chemical cerebronic acid. For example, an increase of the cerebronic acid of at least 1 percent, particularly in a range of 22 to 61-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a b0801-protein, or if the activity of the polypeptide B0801, preferably represented by SEQ ID NO. 75808, or a homolog or fragment thereof, or if the activity of a polypeptide encoded by a nucleic acid molecule comprising the nucleic acid SEQ ID NO. 75807, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 75807 or polypeptide SEQ ID NO. 75808, respectively, or a homolog or a fragment thereof, is increased or generated, or if the activity b0801-protein is increased or generated in a non-human organism, like a microorganism or a plant cell, plant or part thereof, especially with non-targeted localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 23 to 41-percent is conferred as compared to a corresponding non-transformed wild type non-human organism.

Accordingly, in one embodiment, an increase of cerebronic acid in a non-human organism, as compared to a corresponding non-transformed wild type non-human organism, is conferred in the process of the invention, if the activity of a polypeptide showing the activity of a b1046-protein, or if the activity of the polypeptide B1046, preferably represented by SEQ ID NO. 124052, or a homolog or fragment thereof, or if the activity of a polypeptide encoded by a nucleic acid molecule comprising the nucleic acid SEQ ID NO. 124051, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 124051 or polypeptide SEQ ID NO. 124052, respectively, or a homolog or a fragment thereof, is increased or generated, or if the activity b1046-protein is increased or generated in a non-human organism, like a microorganism or a plant cell, plant or part thereof, especially with plastidic localization, whereby the respective line discloses in column 7 the fine chemical cerebronic acid. For example, an increase of the cerebronic acid of at least 1 percent, particularly in a range of 24 to 69-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a malonyl CoA-acyl carrier protein transacylase, or if the activity of the polypeptide B1092, preferably represented by SEQ ID NO. 124128, or a homolog or fragment thereof, or if the activity of a polypeptide encoded by a nucleic acid molecule comprising the nucleic acid SEQ ID NO. 124127, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 124127 or polypeptide SEQ ID NO. 124128, respectively, or a homolog or a fragment thereof, is increased or generated, or if the activity malonyl CoA-acyl carrier protein transacylase is increased or generated in a non-human organism, like a microorganism or a plant cell, plant or part thereof, especially with plastidic localization, whereby the respective line discloses in column 7 the fine chemical hexadecatrienoic acid. For example, an increase of the hexadecatrienoic acid of at least 1 percent, particularly in a range of 12 to 21-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a 3-oxoacyl-[acyl-carrier-protein] reductase, or if the activity of the polypeptide B1093, preferably represented by SEQ ID NO. 118372, or a homolog or fragment thereof, or if the activity of a polypeptide encoded by a nucleic acid molecule comprising the nucleic acid SEQ ID NO. 118371, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 118371 or polypeptide SEQ ID NO. 118372, respectively, or a homolog or a fragment thereof, is increased or generated, or if the activity 3-oxoacyl-[acyl-carrier-protein] reductase is increased or generated in a non-human organism, like a microorganism or a plant cell, plant or part thereof, especially with non-targeted localization, whereby the respective line discloses in column 7 the fine chemical hexadecadienoic acid. For example, an increase of the hexadecadienoic acid of at least 1 percent, particularly in a range of 23 to 30-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a 3-oxoacyl-[acyl-carrier-protein] reductase, or if the activity of the polypeptide B1093, preferably represented by SEQ ID NO. 118372, or a homolog or fragment thereof, or if the activity of a polypeptide encoded by a nucleic acid molecule comprising the nucleic acid SEQ ID NO. 118371, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 118371 or polypeptide SEQ ID NO. 118372, respectively, or a homolog or a fragment thereof, is increased or generated, or if the activity 3-oxoacyl-[acyl-carrier-protein] reductase is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 12 to 18-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a 3-oxoacyl-[acyl-carrier-protein] synthase, or if the activity of the polypeptide B1095, preferably represented by SEQ ID NO. 114553, or a homolog or fragment thereof, or if the activity of a polypeptide encoded by a nucleic acid molecule comprising the nucleic acid SEQ ID NO. 114552, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 114552 or polypeptide SEQ ID NO. 114553, respectively, or a homolog or a fragment thereof, is increased or generated, or if the activity 3-oxoacyl-[acyl-carrier-protein] synthase is increased or generated in a non-human organism, like a microorganism or a plant cell, plant or part thereof, especially with plastidic localization, whereby the respective line discloses in column 7 the fine chemical hexadecatrienoic acid. For example, an increase of the hexadecatrienoic acid of at least 1 percent, particularly in a range of 15 to 44-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a 3-oxoacyl-[acyl-carrier-protein] synthase, or if the activity of the polypeptide B1095, preferably represented by SEQ ID NO. 114553, or a homolog or fragment thereof, or if the activity of a polypeptide encoded by a nucleic acid molecule comprising the nucleic acid SEQ ID NO. 114552, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 114552 or polypeptide SEQ ID NO. 114553, respectively, or a homolog or a fragment thereof, is increased or generated, or if the activity 3-oxoacyl-[acyl-carrier-protein] synthase is increased or generated in a non-human organism, like a microorganism or a plant cell, plant or part thereof, especially with plastidic localization, whereby the respective line discloses in column 7 the fine chemical 2-hydroxybehenic acid. For example, an increase of the 2-hydroxybehenic acid of at least 1 percent, particularly in a range of 27 to 73-percent is conferred as compared to a corresponding non-transformed wild type non-human organism.

Accordingly, in one embodiment, an increase of oleic acid in a non-human organism, as compared to a corresponding non-transformed wild type non-human organism, is conferred in the process of the invention, if the activity of a polypeptide showing the activity of a sodium/proton antiporter, or if the activity of the polypeptide B1186, preferably represented by SEQ ID NO. 37401, or a homolog or fragment thereof, or if the activity of a polypeptide encoded by a nucleic acid molecule comprising the nucleic acid SEQ ID NO. 37400, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 37400 or polypeptide SEQ ID NO. 37401, respectively, or a homolog or a fragment thereof, is increased or generated, or if the activity sodium/proton antiporter is increased or generated in a non-human organism, like a microorganism or a plant cell, plant or part thereof, especially with non-targeted localization, whereby the respective line discloses in column 7 the fine chemical oleic acid. For example, an increase of the oleic acid of at least 1 percent, particularly in a range of 26 to 110-percent is conferred as compared to a corresponding non-transformed wild type non-human organism.

Accordingly, in one embodiment, an increase of 2-hydroxypalmitic acid in a non-human organism, as compared to a corresponding non-transformed wild type non-human organism, is conferred in the process of the invention, if the activity of a polypeptide showing the activity of a sodium/proton antiporter, or if the activity of the polypeptide B1186, preferably represented by SEQ ID NO. 37401, or a homolog or fragment thereof, or if the activity of a polypeptide encoded by a nucleic acid molecule comprising the nucleic acid SEQ ID NO. 37400, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 37400 or polypeptide SEQ ID NO. 37401, respectively, or a homolog or a fragment thereof, is increased or generated, or if the activity sodium/proton antiporter is increased or generated in a non-human organism, like a microorganism or a plant cell, plant or part thereof, especially with non-targeted localization, whereby the respective line discloses in column 7 the fine chemical 2-hydroxypalmitic acid.

For example, an increase of the 2-hydroxypalmitic acid of at least 1 percent, particularly in a range of 19 to 45-percent is conferred as compared to a corresponding non-transformed wild type non-human organism.

Accordingly, in one embodiment, an increase of behenic acid in a non-human organism, as compared to a corresponding non-transformed wild type non-human organism, is conferred in the process of the invention, if the activity of a polypeptide showing the activity of a outermembrane lipoprotein precursor, or if the activity of the polypeptide B1209, preferably represented by SEQ ID NO. 124510, or a homolog or fragment thereof, or if the activity of a polypeptide encoded by a nucleic acid molecule comprising the nucleic acid SEQ ID NO. 124509, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 124509 or polypeptide SEQ ID NO. 124510, respectively, or a homolog or a fragment thereof, is increased or generated, or if the activity outer-membrane lipoprotein precursor is increased or generated in a non-human organism, like a microorganism or a plant cell, plant or part thereof, especially with plastidic localization, whereby the respective line discloses in column 7 the fine chemical behenic acid. For example, an increase of the behenic acid of at least 1 percent, particularly in a range of 9 to 16-percent is conferred as compared to a corresponding non-transformed wild type non-human organism.

Accordingly, in one embodiment, an increase of behenic acid in a non-human organism, as compared to a corresponding non-transformed wild type non-human organism, is conferred in the process of the invention, if the activity of a polypeptide showing the activity of a cardiolipin synthase, or if the activity of the polypeptide B1249, preferably represented by SEQ ID NO. 98779, or a homolog or fragment thereof, or if the activity of a polypeptide encoded by a nucleic acid molecule comprising the nucleic acid SEQ ID NO. 98778, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 98778 or polypeptide SEQ ID NO. 98779, respectively, or a homolog or a fragment thereof, is increased or generated, or if the activity cardiolipin synthase is increased or generated in a non-human organism, like a microorganism or a plant cell, plant or part thereof, especially with plastidic localization, whereby the respective line discloses in column 7 the fine chemical behenic acid. For example, an increase of the behenic acid of at least 1 percent, particularly in a range of 9 to 15-percent is conferred as compared to a corresponding non-transformed wild type non-human organism.

Accordingly, in one embodiment, an increase of heptadecanoic acid in a non-human organism, as compared to a corresponding non-transformed wild type non-human organism, is conferred in the process of the invention, if the activity of a polypeptide showing the activity of a cardiolipin synthase, or if the activity of the polypeptide B1249, preferably represented by SEQ ID NO. 98779, or a homolog or fragment thereof, or if the activity of a polypeptide encoded by a nucleic acid molecule comprising the nucleic acid SEQ ID NO. 98778, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 98778 or polypeptide SEQ ID NO. 98779, respectively, or a homolog or a fragment thereof, is increased or generated, or if the activity cardiolipin synthase is increased or generated in a non-human organism, like a microorganism or a plant cell, plant or part thereof, especially with plastidic localization, whereby the respective line discloses in column 7 the fine chemical heptadecanoic acid. For example, an increase of the heptadecanoic acid of at least 1 percent, particularly in a range of 13 to 25-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a b1346-protein, or if the activity of the polypeptide B1346, preferably represented by SEQ ID NO. 99189, or a homolog or fragment thereof, or if the activity of a polypeptide encoded by a nucleic acid molecule comprising the nucleic acid SEQ ID NO. 99188, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 99188 or polypeptide SEQ ID NO. 99189, respectively, or a homolog or a fragment thereof, is increased or generated, or if the activity b1346-protein is increased or generated in a non-human organism, like a microorganism or a plant cell, plant or part thereof, especially with plastidic localization, whereby the respective line discloses in column 7 the fine chemical hexadecatrienoic acid. For example, an increase of the hexadecatrienoic acid of at least 1 percent, particularly in a range of 15 to 43-percent is conferred as compared to a corresponding non-transformed wild type non-human organism.

Accordingly, in one embodiment, an increase of lauric acid in a non-human organism, as compared to a corresponding non-transformed wild type non-human organism, is conferred in the process of the invention, if the activity of a polypeptide showing the activity of a phage-related repressor protein, or if the activity of the polypeptide B1356, preferably represented by SEQ ID NO. 124558, or a homolog or fragment thereof, or if the activity of a polypeptide encoded by a nucleic acid molecule comprising the nucleic acid SEQ ID NO. 124557, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 124557 or polypeptide SEQ ID NO. 124558, respectively, or a homolog or a fragment thereof, is increased or generated, or if the activity phage-related repressor protein is increased or generated in a non-human organism, like a microorganism or a plant cell, plant or part thereof, especially with non-targeted localization, whereby the respective line discloses in column 7 the fine chemical lauric acid. For example, an increase of the lauric acid of at least 1 percent, particularly in a range of 45 to 61-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a lipoprotein precursor, or if the activity of the polypeptide B1431, preferably represented by SEQ ID NO. 38267, or a homolog or fragment thereof, or if the activity of a polypeptide encoded by a nucleic acid molecule comprising the nucleic acid SEQ ID NO. 38266, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 38266 or polypeptide SEQ ID NO. 38267, respectively, or a homolog or a fragment thereof, is increased or generated, or if the activity lipoprotein precursor is increased or generated in a non-human organism, like a microorganism or a plant cell, plant or part thereof, especially with plastidic localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 25 to 94-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a b1470-protein, or if the activity of the polypeptide B1470, preferably represented by SEQ ID NO. 115057, or a homolog or fragment thereof, or if the activity of a polypeptide encoded by a nucleic acid molecule comprising the nucleic acid SEQ ID NO. 115056, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 115056 or polypeptide SEQ ID NO. 115057, respectively, or a homolog or a fragment thereof, is increased or generated, or if the activity b1470-protein is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 16 to 25-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a NADP-dependent malic enzyme, or if the activity of the polypeptide B1479, preferably represented by SEQ ID NO. 77775, or a homolog or fragment thereof, or if the activity of a polypeptide encoded by a nucleic acid molecule comprising the nucleic acid SEQ ID NO. 77774, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 77774 or polypeptide SEQ ID NO. 77775, respectively, or a homolog or a fragment thereof, is increased or generated, or if the activity NADP-dependent malic enzyme is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 17 to 49-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a b1522-protein, or if the activity of the polypeptide B1522, preferably represented by SEQ ID NO. 7948, or a homolog or fragment thereof, or if the activity of a polypeptide encoded by a nucleic acid molecule comprising the nucleic acid SEQ ID NO. 7947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 7947 or polypeptide SEQ ID NO. 7948, respectively, or a homolog or a fragment thereof, is increased or generated, or if the activity b1522-protein is increased or generated in a non-human organism, like a microorganism or a plant cell, plant or part thereof, especially with non-targeted localization, whereby the respective line discloses in column 7 the fine chemical 2-hydroxybehenic acid. For example, an increase of the 2-hydroxybehenic acid of at least 1 percent, particularly in a range of 26 to 81-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a acid shock protein, or if the activity of the polypeptide B1597, preferably represented by SEQ ID NO. 38301, or a homolog or fragment thereof, or if the activity of a polypeptide encoded by a nucleic acid molecule comprising the nucleic acid SEQ ID NO. 38300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 38300 or polypeptide SEQ ID NO. 38301, respectively, or a homolog or a fragment thereof, is increased or generated, or if the activity acid shock protein is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 12 to 48-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a transport protein, or if the activity of the polypeptide B1601, preferably represented by SEQ ID NO. 7993, or a homolog or fragment thereof, or if the activity of a polypeptide encoded by a nucleic acid molecule comprising the nucleic acid SEQ ID NO. 7992, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 7992 or polypeptide SEQ ID NO. 7993, respectively, or a homolog or a fragment thereof, is increased or generated, or if the activity transport protein is increased or generated in a non-human organism, like a microorganism or a plant cell, plant or part thereof, especially with non-targeted localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 22 to 43-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a phosphotransferase system component, or if the activity of the polypeptide B1621, preferably represented by SEQ ID NO. 124572, or a homolog or fragment thereof, or if the activity of a polypeptide encoded by a nucleic acid molecule comprising the nucleic acid SEQ ID NO. 124571, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 124571 or polypeptide SEQ ID NO. 124572, respectively, or a homolog or a fragment thereof, is increased or generated, or if the activity phosphotransferase system component is increased or generated in a non-human organism, like a microorganism or a plant cell, plant or part thereof, especially with plastidic localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 25 to 35-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a electron transport complex protein, or if the activity of the polypeptide B1627, preferably represented by SEQ ID NO. 38346, or a homolog or fragment thereof, or if the activity of a polypeptide encoded by a nucleic acid molecule comprising the nucleic acid SEQ ID NO. 38345, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 38345 or polypeptide SEQ ID NO. 38346, respectively, or a homolog or a fragment thereof, is increased or generated, or if the activity electron transport complex protein is increased or generated in a non-human organism, like a microorganism or a plant cell, plant or part thereof, especially with plastidic localization, whereby the respective line discloses in column 7 the fine chemical 2-hydroxybehenic acid. For example, an increase of the 2-hydroxybehenic acid of at least 1 percent, particularly in a range of 30 to 36-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a pyridoxamine kinase, or if the activity of the polypeptide B1636, preferably represented by SEQ ID NO. 124806, or a homolog or fragment thereof, or if the activity of a polypeptide encoded by a nucleic acid molecule comprising the nucleic acid SEQ ID NO. 124805, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 124805 or polypeptide SEQ ID NO. 124806, respectively, or a homolog or a fragment thereof, is increased or generated, or if the activity pyridoxamine kinase is increased or generated in a non-human organism, like a microorganism or a plant cell, plant or part thereof, especially with plastidic localization, whereby the respective line discloses in column 7 the fine chemical hexadecatrienoic acid. For example, an increase of the hexadecatrienoic acid of at least 1 percent, particularly in a range of 17 to 28-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a b1670-protein, or if the activity of the polypeptide B1670, preferably represented by SEQ ID NO. 78754, or a homolog or fragment thereof, or if the activity of a polypeptide encoded by a nucleic acid molecule comprising the nucleic acid SEQ ID NO. 78753, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 78753 or polypeptide SEQ ID NO. 78754, respectively, or a homolog or a fragment thereof, is increased or generated, or if the activity b1670-protein is increased or generated in a non-human organism, like a microorganism or a plant cell, plant or part thereof, especially with non-targeted localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 23 to 63-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a b1703-protein, or if the activity of the polypeptide B1703, preferably represented by SEQ ID NO. 124908, or a homolog or fragment thereof, or if the activity of a polypeptide encoded by a nucleic acid molecule comprising the nucleic acid SEQ ID NO. 124907, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 124907 or polypeptide SEQ ID NO. 124908, respectively, or a homolog or a fragment thereof, is increased or generated, or if the activity b1703-protein is increased or generated in a non-human organism, like a microorganism or a plant cell, plant or part thereof, especially with non-targeted localization, whereby the respective line discloses in column 7 the fine chemical hexadecadienoic acid. For example, an increase of the hexadecadienoic acid of at least 1 percent, particularly in a range of 28 to 66-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a b1703-protein, or if the activity of the polypeptide B1703, preferably represented by SEQ ID NO. 124908, or a homolog or fragment thereof, or if the activity of a polypeptide encoded by a nucleic acid molecule comprising the nucleic acid SEQ ID NO. 124907, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 124907 or polypeptide SEQ ID NO. 124908, respectively, or a homolog or a fragment thereof, is increased or generated, or if the activity b1703-protein is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 14 to 24-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a transport protein, or if the activity of the polypeptide B1791, preferably represented by SEQ ID NO. 78884, or a homolog or fragment thereof, or if the activity of a polypeptide encoded by a nucleic acid molecule comprising the nucleic acid SEQ ID NO. 78883, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 78883 or polypeptide SEQ ID NO. 78884, respectively, or a homolog or a fragment thereof, is increased or generated, or if the activity transport protein is increased or generated in a non-human organism, like a microorganism or a plant cell, plant or part thereof, especially with non-targeted localization, whereby the respective line discloses in column 7 the fine chemical hexadecadienoic acid. For example, an increase of the hexadecadienoic acid of at least 1 percent, particularly in a range of 46 to 64-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a b1837-protein, or if the activity of the polypeptide B1837, preferably represented by SEQ ID NO. 78954, or a homolog or fragment thereof, or if the activity of a polypeptide encoded by a nucleic acid molecule comprising the nucleic acid SEQ ID NO. 78953, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 78953 or polypeptide SEQ ID NO. 78954, respectively, or a homolog or a fragment thereof, is increased or generated, or if the activity b1837-protein is increased or generated in a non-human organism, like a microorganism or a plant cell, plant or part thereof, especially with non-targeted localization, whereby the respective line discloses in column 7 the fine chemical hexadecadienoic acid. For example, an increase of the hexadecadienoic acid of at least 1 percent, particularly in a range of 31 to 35-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a b1837-protein, or if the activity of the polypeptide B1837, preferably represented by SEQ ID NO. 78954, or a homolog or fragment thereof, or if the activity of a polypeptide encoded by a nucleic acid molecule comprising the nucleic acid SEQ ID NO. 78953, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 78953 or polypeptide SEQ ID NO. 78954, respectively, or a homolog or a fragment thereof, is increased or generated, or if the activity b1837-protein is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 15 to 27-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a b2032-protein, or if the activity of the polypeptide B2032, preferably represented by SEQ ID NO. 8921, or a homolog or fragment thereof, or if the activity of a polypeptide encoded by a nucleic acid molecule comprising the nucleic acid SEQ ID NO. 8920, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 8920 or polypeptide SEQ ID NO. 8921, respectively, or a homolog or a fragment thereof, is increased or generated, or if the activity b2032-protein is increased or generated in a non-human organism, like a microorganism or a plant cell, plant or part thereof, especially with non-targeted localization, whereby the respective line discloses in column 7 the fine chemical hexadecadienoic acid. For example, an increase of the hexadecadienoic acid of at least 1 percent, particularly in a range of 57 to 88-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a b2099-protein, or if the activity of the polypeptide B2099, preferably represented by SEQ ID NO. 115596, or a homolog or fragment thereof, or if the activity of a polypeptide encoded by a nucleic acid molecule comprising the nucleic acid SEQ ID NO. 115595, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 115595 or polypeptide SEQ ID NO. 115596, respectively, or a homolog or a fragment thereof, is increased or generated, or if the activity b2099-protein is increased or generated in a non-human organism, like a microorganism or a plant cell, plant or part thereof, especially with non-targeted localization, whereby the respective line discloses in column 7 the fine chemical hexadecadienoic acid. For example, an increase of the hexadecadienoic acid of at least 1 percent, particularly in a range of 36 to 47-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a b2099-protein, or if the activity of the polypeptide B2099, preferably represented by SEQ ID NO. 115596, or a homolog or fragment thereof, or if the activity of a polypeptide encoded by a nucleic acid molecule comprising the nucleic acid SEQ ID NO. 115595, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 115595 or polypeptide SEQ ID NO. 115596, respectively, or a homolog or a fragment thereof, is increased or generated, or if the activity b2099-protein is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 18 to 24-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a b2107-protein, or if the activity of the polypeptide B2107, preferably represented by SEQ ID NO. 39003, or a homolog or fragment thereof, or if the activity of a polypeptide encoded by a nucleic acid molecule comprising the nucleic acid SEQ ID NO. 39002, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 39002 or polypeptide SEQ ID NO. 39003, respectively, or a homolog or a fragment thereof, is increased or generated, or if the activity b2107-protein is increased or generated in a non-human organism, like a microorganism or a plant cell, plant or part thereof, especially with non-targeted localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 24 to 39-percent is conferred as compared to a corresponding non-transformed wild type non-human organism.

Accordingly, in one embodiment, an increase of lauric acid in a non-human organism, as compared to a corresponding non-transformed wild type non-human organism, is conferred in the process of the invention, if the activity of a polypeptide showing the activity of a b2126-protein, or if the activity of the polypeptide B2126, preferably represented by SEQ ID NO. 124993, or a homolog or fragment thereof, or if the activity of a polypeptide encoded by a nucleic acid molecule comprising the nucleic acid SEQ ID NO. 124992, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 124992 or polypeptide SEQ ID NO. 124993, respectively, or a homolog or a fragment thereof, is increased or generated, or if the activity b2126-protein is increased or generated in a non-human organism, like a microorganism or a plant cell, plant or part thereof, especially with plastidic localization, whereby the respective line discloses in column 7 the fine chemical lauric acid. For example, an increase of the lauric acid of at least 1 percent, particularly in a range of 32 to 43-percent is conferred as compared to a corresponding non-transformed wild type non-human organism.

Accordingly, in one embodiment, an increase of 2-hydroxypalmitic acid in a non-human organism, as compared to a corresponding non-transformed wild type non-human organism, is conferred in the process of the invention, if the activity of a polypeptide showing the activity of a b2139-protein, or if the activity of the polypeptide B2139, preferably represented by SEQ ID NO. 125086, or a homolog or fragment thereof, or if the activity of a polypeptide encoded by a nucleic acid molecule comprising the nucleic acid SEQ ID NO. 125085, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 125085 or polypeptide SEQ ID NO. 125086, respectively, or a homolog or a fragment thereof, is increased or generated, or if the activity b2139-protein is increased or generated in a non-human organism, like a microorganism or a plant cell, plant or part thereof, especially with plastidic localization, whereby the respective line discloses in column 7 the fine chemical 2-hydroxypalmitic acid. For example, an increase of the 2-hydroxypalmitic acid of at least 1 percent, particularly in a range of 20 to 27-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a ABC transporter permease protein, or if the activity of the polypeptide B2178, preferably represented by SEQ ID NO. 39041, or a homolog or fragment thereof, or if the activity of a polypeptide encoded by a nucleic acid molecule comprising the nucleic acid SEQ ID NO. 39040, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 39040 or polypeptide SEQ ID NO. 39041, respectively, or a homolog or a fragment thereof, is increased or generated, or if the activity ABC transporter permease protein is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 17 to 34-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a sensor kinase, or if the activity of the polypeptide B2216, preferably represented by SEQ ID NO. 125106, or a homolog or fragment thereof, or if the activity of a polypeptide encoded by a nucleic acid molecule comprising the nucleic acid SEQ ID NO. 125105, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 125105 or polypeptide SEQ ID NO. 125106, respectively, or a homolog or a fragment thereof, is increased or generated, or if the activity sensor kinase is increased or generated in a non-human organism, like a microorganism or a plant cell, plant or part thereof, especially with plastidic localization, whereby the respective line discloses in column 7 the fine chemical 2-hydroxybehenic acid. For example, an increase of the 2-hydroxybehenic acid of at least 1 percent, particularly in a range of 24 to 34-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a b2360-protein, or if the activity of the polypeptide B2360, preferably represented by SEQ ID NO. 39220, or a homolog or fragment thereof, or if the activity of a polypeptide encoded by a nucleic acid molecule comprising the nucleic acid SEQ ID NO. 39219, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 39219 or polypeptide SEQ ID NO. 39220, respectively, or a homolog or a fragment thereof, is increased or generated, or if the activity b2360-protein is increased or generated in a non-human organism, like a microorganism or a plant cell, plant or part thereof, especially with non-targeted localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 33 to 57-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a cysteine synthase A, or if the activity of the polypeptide B2414, preferably represented by SEQ ID NO. 39301, or a homolog or fragment thereof, or if the activity of a polypeptide encoded by a nucleic acid molecule comprising the nucleic acid SEQ ID NO. 39300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 39300 or polypeptide SEQ ID NO. 39301, respectively, or a homolog or a fragment thereof, is increased or generated, or if the activity cysteine synthase A is increased or generated in a non-human organism, like a microorganism or a plant cell, plant or part thereof, especially with non-targeted localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 31 to 60-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a ethanolamine utilization protein, or if the activity of the polypeptide B2439, preferably represented by SEQ ID NO. 115629, or a homolog or fragment thereof, or if the activity of a polypeptide encoded by a nucleic acid molecule comprising the nucleic acid SEQ ID NO. 115628, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 115628 or polypeptide SEQ ID NO. 115629, respectively, or a homolog or a fragment thereof, is increased or generated, or if the activity ethanolamine utilization protein is increased or generated in a non-human organism, like a microorganism or a plant cell, plant or part thereof, especially with non-targeted localization, whereby the respective line discloses in column 7 the fine chemical hexadecadienoic acid. For example, an increase of the hexadecadienoic acid of at least 1 percent, particularly in a range of 32 to 135-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a ethanolamine utilization protein, or if the activity of the polypeptide B2439, preferably represented by SEQ ID NO. 115629, or a homolog or fragment thereof, or if the activity of a polypeptide encoded by a nucleic acid molecule comprising the nucleic acid SEQ ID NO. 115628, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 115628 or polypeptide SEQ ID NO. 115629, respectively, or a homolog or a fragment thereof, is increased or generated, or if the activity ethanolamine utilization protein is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 14 to 27-percent is conferred as compared to a corresponding non-transformed wild type non-human organism.

Accordingly, in one embodiment, an increase of oleic acid in a non-human organism, as compared to a corresponding non-transformed wild type non-human organism, is conferred in the process of the invention, if the activity of a polypeptide showing the activity of a ethanolamine utilization protein, or if the activity of the polypeptide B2439, preferably represented by SEQ ID NO. 115629, or a homolog or fragment thereof, or if the activity of a polypeptide encoded by a nucleic acid molecule comprising the nucleic acid SEQ ID NO. 115628, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 115628 or polypeptide SEQ ID NO. 115629, respectively, or a homolog or a fragment thereof, is increased or generated, or if the activity ethanolamine utilization protein is increased or generated in a non-human organism, like a microorganism or a plant cell, plant or part thereof, especially with non-targeted localization, whereby the respective line discloses in column 7 the fine chemical oleic acid. For example, an increase of the oleic acid of at least 1 percent, particularly in a range of 42 to 55-percent is conferred as compared to a corresponding non-transformed wild type non-human organism.

Accordingly, in one embodiment, an increase of cerebronic acid in a non-human organism, as compared to a corresponding non-transformed wild type non-human organism, is conferred in the process of the invention, if the activity of a polypeptide showing the activity of a b2474-protein, or if the activity of the polypeptide B2474, preferably represented by SEQ ID NO. 40330, or a homolog or fragment thereof, or if the activity of a polypeptide encoded by a nucleic acid molecule comprising the nucleic acid SEQ ID NO. 40329, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 40329 or polypeptide SEQ ID NO. 40330, respectively, or a homolog or a fragment thereof, is increased or generated, or if the activity b2474-protein is increased or generated in a non-human organism, like a microorganism or a plant cell, plant or part thereof, especially with non-targeted localization, whereby the respective line discloses in column 7 the fine chemical cerebronic acid. For example, an increase of the cerebronic acid of at least 1 percent, particularly in a range of 25 to 44-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a b2474-protein, or if the activity of the polypeptide B2474, preferably represented by SEQ ID NO. 40330, or a homolog or fragment thereof, or if the activity of a polypeptide encoded by a nucleic acid molecule comprising the nucleic acid SEQ ID NO. 40329, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 40329 or polypeptide SEQ ID NO. 40330, respectively, or a homolog or a fragment thereof, is increased or generated, or if the activity b2474-protein is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 20 to 31-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a b2474-protein, or if the activity of the polypeptide B2474, preferably represented by SEQ ID NO. 40330, or a homolog or fragment thereof, or if the activity of a polypeptide encoded by a nucleic acid molecule comprising the nucleic acid SEQ ID NO. 40329, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 40329 or polypeptide SEQ ID NO. 40330, respectively, or a homolog or a fragment thereof, is increased or generated, or if the activity b2474-protein is increased or generated in a non-human organism, like a microorganism or a plant cell, plant or part thereof, especially with non-targeted localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 27 to 43-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a b2513-protein, or if the activity of the polypeptide B2513, preferably represented by SEQ ID NO. 9168, or a homolog or fragment thereof, or if the activity of a polypeptide encoded by a nucleic acid molecule comprising the nucleic acid SEQ ID NO. 9167, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 9167 or polypeptide SEQ ID NO. 9168, respectively, or a homolog or a fragment thereof, is increased or generated, or if the activity b2513-protein is increased or generated in a non-human organism, like a microorganism or a plant cell, plant or part thereof, especially with non-targeted localization, whereby the respective line discloses in column 7 the fine chemical hexadecadienoic acid. For example, an increase of the hexadecadienoic acid of at least 1 percent, particularly in a range of 26 to 81-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a phosphatidylserine synthase, or if the activity of the polypeptide B2585, preferably represented by SEQ ID NO. 125143, or a homolog or fragment thereof, or if the activity of a polypeptide encoded by a nucleic acid molecule comprising the nucleic acid SEQ ID NO. 125142, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 125142 or polypeptide SEQ ID NO. 125143, respectively, or a homolog or a fragment thereof, is increased or generated, or if the activity phosphatidylserine synthase is increased or generated in a non-human organism, like a microorganism or a plant cell, plant or part thereof, especially with non-targeted localization, whereby the respective line discloses in column 7 the fine chemical hexadecadienoic acid. For example, an increase of the hexadecadienoic acid of at least 1 percent, particularly in a range of 21 to 32-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a b2654-protein, or if the activity of the polypeptide B2654, preferably represented by SEQ ID NO. 125227, or a homolog or fragment thereof, or if the activity of a polypeptide encoded by a nucleic acid molecule comprising the nucleic acid SEQ ID NO. 125226, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 125226 or polypeptide SEQ ID NO. 125227, respectively, or a homolog or a fragment thereof, is increased or generated, or if the activity b2654-protein is increased or generated in a non-human organism, like a microorganism or a plant cell, plant or part thereof, especially with non-targeted localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 38 to 41-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a 2-C-methyl-D-erythritol 4-phosphate cytidylyltransferase, or if the activity of the polypeptide B2747, preferably represented by SEQ ID NO. 81076, or a homolog or fragment thereof, or if the activity of a polypeptide encoded by a nucleic acid molecule comprising the nucleic acid SEQ ID NO. 81075, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 81075 or polypeptide SEQ ID NO. 81076, respectively, or a homolog or a fragment thereof, is increased or generated, or if the activity 2-C-methyl-D-erythritol 4-phosphate cytidylyltransferase is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 13 to 45-percent is conferred as compared to a corresponding non-transformed wild type non-human organism.

Accordingly, in one embodiment, an increase of eicosanoic acid in a non-human organism, as compared to a corresponding non-transformed wild type non-human organism, is conferred in the process of the invention, if the activity of a polypeptide showing the activity of a alkaline phosphatase isozyme conversion protein, or if the activity of the polypeptide B2753, preferably represented by SEQ ID NO. 125235, or a homolog or fragment thereof, or if the activity of a polypeptide encoded by a nucleic acid molecule comprising the nucleic acid SEQ ID NO. 125234, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 125234 or polypeptide SEQ ID NO. 125235, respectively, or a homolog or a fragment thereof, is increased or generated, or if the activity alkaline phosphatase isozyme conversion protein is increased or generated in a non-human organism, like a microorganism or a plant cell, plant or part thereof, especially with non-targeted localization, whereby the respective line discloses in column 7 the fine chemical eicosanoic acid. For example, an increase of the eicosanoic acid of at least 1 percent, particularly in a range of 10 to 22-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a murein transglycosylase, or if the activity of the polypeptide B2963, preferably represented by SEQ ID NO. 41733, or a homolog or fragment thereof, or if the activity of a polypeptide encoded by a nucleic acid molecule comprising the nucleic acid SEQ ID NO. 41732, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 41732 or polypeptide SEQ ID NO. 41733, respectively, or a homolog or a fragment thereof, is increased or generated, or if the activity murein transglycosylase is increased or generated in a non-human organism, like a microorganism or a plant cell, plant or part thereof, especially with plastidic localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 25 to 55-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a b3442-protein, or if the activity of the polypeptide B3442, preferably represented by SEQ ID NO. 94381, or a homolog or fragment thereof, or if the activity of a polypeptide encoded by a nucleic acid molecule comprising the nucleic acid SEQ ID NO. 94380, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 94380 or polypeptide SEQ ID NO. 94381, respectively, or a homolog or a fragment thereof, is increased or generated, or if the activity b3442-protein is increased or generated in a non-human organism, like a microorganism or a plant cell, plant or part thereof, especially with non-targeted localization, whereby the respective line discloses in column 7 the fine chemical hexadecadienoic acid. For example, an increase of the hexadecadienoic acid of at least 1 percent, particularly in a range of 26 to 102-percent is conferred as compared to a corresponding non-transformed wild type non-human organism.

Accordingly, in one embodiment, an increase of cerebronic acid in a non-human organism, as compared to a corresponding non-transformed wild type non-human organism, is conferred in the process of the invention, if the activity of a polypeptide showing the activity of a sugar transport system permease protein, or if the activity of the polypeptide B3568, preferably represented by SEQ ID NO. 125265, or a homolog or fragment thereof, or if the activity of a polypeptide encoded by a nucleic acid molecule comprising the nucleic acid SEQ ID NO. 125264, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 125264 or polypeptide SEQ ID NO. 125265, respectively, or a homolog or a fragment thereof, is increased or generated, or if the activity sugar transport system permease protein is increased or generated in a non-human organism, like a microorganism or a plant cell, plant or part thereof, especially with plastidic localization, whereby the respective line discloses in column 7 the fine chemical cerebronic acid. For example, an increase of the cerebronic acid of at least 1 percent, particularly in a range of 30 to 44-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a b3812-protein, or if the activity of the polypeptide B3812, preferably represented by SEQ ID NO. 125488, or a hornolog or fragment thereof, or if the activity of a polypeptide encoded by a nucleic acid molecule comprising the nucleic acid SEQ ID NO. 125487, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 125487 or polypeptide SEQ ID NO. 125488, respectively, or a homolog or a fragment thereof, is increased or generated, or if the activity b3812-protein is increased or generated in a non-human organism, like a microorganism or a plant cell, plant or part thereof, especially with non-targeted localization, whereby the respective line discloses in column 7 the fine chemical 2-hydroxybehenic acid. For example, an increase of the 2-hydroxybehenic acid of at least 1 percent, particularly in a range of 29 to 65-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a 3-ketoacyl-CoA thiolase, or if the activity of the polypeptide B3845, preferably represented by SEQ ID NO. 100676, or a homolog or fragment thereof, or if the activity of a polypeptide encoded by a nucleic acid molecule comprising the nucleic acid SEQ ID NO. 100675, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 100675 or polypeptide SEQ ID NO. 100676, respectively, or a homolog or a fragment thereof, is increased or generated, or if the activity 3-ketoacyl-CoA thiolase is increased or generated in a non-human organism, like a microorganism or a plant cell, plant or part thereof, especially with non-targeted localization, whereby the respective line discloses in column 7 the fine chemical hexadecadienoic acid. For example, an increase of the hexadecadienoic acid of at least 1 percent, particularly in a range of 20 to 36-percent is conferred as compared to a corresponding non-transformed wild type non-human organism.

Accordingly, in one embodiment, an increase of oleic acid in a non-human organism, as compared to a corresponding non-transformed wild type non-human organism, is conferred in the process of the invention, if the activity of a polypeptide showing the activity of a 3-ketoacyl-CoA thiolase, or if the activity of the polypeptide B3845, preferably represented by SEQ ID NO. 100676, or a homolog or fragment thereof, or if the activity of a polypeptide encoded by a nucleic acid molecule comprising the nucleic acid SEQ ID NO. 100675, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 100675 or polypeptide SEQ ID NO. 100676, respectively, or a homolog or a fragment thereof, is increased or generated, or if the activity 3-ketoacyl-CoA thiolase is increased or generated in a non-human organism, like a microorganism or a plant cell, plant or part thereof, especially with plastidic localization, whereby the respective line discloses in column 7 the fine chemical oleic acid. For example, an increase of the oleic acid of at least 1 percent, particularly in a range of 26 to 30-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a transcriptional regulator, or if the activity of the polypeptide B3872, preferably represented by SEQ ID NO. 83822, or a homolog or fragment thereof, or if the activity of a polypeptide encoded by a nucleic acid molecule comprising the nucleic acid SEQ ID NO. 83821, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 83821 or polypeptide SEQ ID NO. 83822, respectively, or a homolog or a fragment thereof, is increased or generated, or if the activity transcriptional regulator is increased or generated in a non-human organism, like a microorganism or a plant cell, plant or part thereof, especially with plastidic localization, whereby the respective line discloses in column 7 the fine chemical hexadecadienoic acid. For example, an increase of the hexadecadienoic acid of at least 1 percent, particularly in a range of 28 to 58-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a fructose 1,6-bisphosphatase, or if the activity of the polypeptide B3925, preferably represented by SEQ ID NO. 125542, or a homolog or fragment thereof, or if the activity of a polypeptide encoded by a nucleic acid molecule comprising the nucleic acid SEQ ID NO. 125541, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 125541 or polypeptide SEQ ID NO. 125542, respectively, or a homolog or a fragment thereof, is increased or generated, or if the activity fructose 1,6-bisphosphatase is increased or generated in a non-human organism, like a microorganism or a plant cell, plant or part thereof, especially with non-targeted localization, whereby the respective line discloses in column 7 the fine chemical hexadecadienoic acid. For example, an increase of the hexadecadienoic acid of at least 1 percent, particularly in a range of 25 to 66-percent is conferred as compared to a corresponding non-transformed wild type non-human organism.

Accordingly, in one embodiment, an increase of behenic acid in a non-human organism, as compared to a corresponding non-transformed wild type non-human organism, is conferred in the process of the invention, if the activity of a polypeptide showing the activity of a cystathionine gamma-synthase, or if the activity of the polypeptide B3939, preferably represented by SEQ ID NO. 125796, or a homolog or fragment thereof, or if the activity of a polypeptide encoded by a nucleic acid molecule comprising the nucleic acid SEQ ID NO. 125795, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 125795 or polypeptide SEQ ID NO. 125796, respectively, or a homolog or a fragment thereof, is increased or generated, or if the activity cystathionine gamma-synthase is increased or generated in a non-human organism, like a microorganism or a plant cell, plant or part thereof, especially with plastidic localization, whereby the respective line discloses in column 7 the fine chemical behenic acid. For example, an increase of the behenic acid of at least 1 percent, particularly in a range of 11 to 25-percent is conferred as compared to a corresponding non-transformed wild type non-human organism.

Accordingly, in one embodiment, an increase of heptadecanoic acid in a non-human organism, as compared to a corresponding non-transformed wild type non-human organism, is conferred in the process of the invention, if the activity of a polypeptide showing the activity of a cystathionine gamma-synthase, or if the activity of the polypeptide B3939, preferably represented by SEQ ID NO. 125796, or a homolog or fragment thereof, or if the activity of a polypeptide encoded by a nucleic acid molecule comprising the nucleic acid SEQ ID NO. 125795, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 125795 or polypeptide SEQ ID NO. 125796, respectively, or a homolog or a fragment thereof, is increased or generated, or if the activity cystathionine gamma-synthase is increased or generated in a non-human organism, like a microorganism or a plant cell, plant or part thereof, especially with plastidic localization, whereby the respective line discloses in column 7 the fine chemical heptadecanoic acid. For example, an increase of the heptadecanoic acid of at least 1 percent, particularly in a range of 22 to 33-percent is conferred as compared to a corresponding non-transformed wild type non-human organism.

Accordingly, in one embodiment, an increase of oleic acid in a non-human organism, as compared to a corresponding non-transformed wild type non-human organism, is conferred in the process of the invention, if the activity of a polypeptide showing the activity of a cystathionine gamma-synthase, or if the activity of the polypeptide B3939, preferably represented by SEQ ID NO. 125796, or a homolog or fragment thereof, or if the activity of a polypeptide encoded by a nucleic acid molecule comprising the nucleic acid SEQ ID NO. 125795, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 125795 or polypeptide SEQ ID NO. 125796, respectively, or a homolog or a fragment thereof, is increased or generated, or if the activity cystathionine gamma-synthase is increased or generated in a non-human organism, like a microorganism or a plant cell, plant or part thereof, especially with plastidic localization, whereby the respective line discloses in column 7 the fine chemical oleic acid. For example, an increase of the oleic acid of at least 1 percent, particularly in a range of 25 to 36-percent is conferred as compared to a corresponding non-transformed wild type non-human organism

Accordingly, in one embodiment, an increase of 2-hydroxypalmitic acid in a non-human organism, as compared to a corresponding non-transformed wild type non-human organism, is conferred in the process of the invention, if the activity of a polypeptide showing the activity of a b3944-protein, or if the activity of the polypeptide B3944, preferably represented by SEQ ID NO. 126378, or a homolog or fragment thereof, or if the activity of a polypeptide encoded by a nucleic acid molecule comprising the nucleic acid SEQ ID NO. 126377, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 126377 or polypeptide SEQ ID NO. 126378, respectively, or a homolog or a fragment thereof, is increased or generated, or if the activity b3944-protein is increased or generated in a non-human organism, like a microorganism or a plant cell, plant or part thereof, especially with plastidic localization, whereby the respective line discloses in column 7 the fine chemical 2-hydroxypalmitic acid. For example, an increase of the 2-hydroxypalmitic acid of at least 1 percent, particularly in a range of 19 to 37-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a ankyrin repeat protein, or if the activity of the polypeptide B4017, preferably represented by SEQ ID NO. 126416, or a homolog or fragment thereof, or if the activity of a polypeptide encoded by a nucleic acid molecule comprising the nucleic acid SEQ ID NO. 126415, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 126415 or polypeptide SEQ ID NO. 126416, respectively, or a homolog or a fragment thereof, is increased or generated, or if the activity ankyrin repeat protein is increased or generated in a non-human organism, like a microorganism or a plant cell, plant or part thereof, especially with plastidic localization, whereby the respective line discloses in column 7 the fine chemical 2-hydroxybehenic acid. For example, an increase of the 2-hydroxybehenic acid of at least 1 percent, particularly in a range of 29 to 49-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a b4023-protein, or if the activity of the polypeptide B4023, preferably represented by SEQ ID NO. 119210, or a homolog or fragment thereof, or if the activity of a polypeptide encoded by a nucleic acid molecule comprising the nucleic acid SEQ ID NO. 119209, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 119209 or polypeptide SEQ ID NO. 119210, respectively, or a homolog or a fragment thereof, is increased or generated, or if the activity b4023-protein is increased or generated in a non-human organism, like a microorganism or a plant cell, plant or part thereof, especially with non-targeted localization, whereby the respective line discloses in column 7 the fine chemical hexadecadienoic acid. For example, an increase of the hexadecadienoic acid of at least 1 percent, particularly in a range of 24 to 57-percent is conferred as compared to a corresponding non-transformed wild type non-human organism.

Accordingly, in one embodiment, an increase of heptadecanoic acid in a non-human organism, as compared to a corresponding non-transformed wild type non-human organism, is conferred in the process of the invention, if the activity of a polypeptide showing the activity of a acetylcoenzyme A synthetase, or if the activity of the polypeptide B4069, preferably represented by SEQ ID NO. 126422, or a homolog or fragment thereof, or if the activity of a polypeptide encoded by a nucleic acid molecule comprising the nucleic acid SEQ ID NO. 126421, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 126421 or polypeptide SEQ ID NO. 126422, respectively, or a homolog or a fragment thereof, is increased or generated, or if the activity acetyl-coenzyme A synthetase is increased or generated in a non-human organism, like a microorganism or a plant cell, plant or part thereof, especially with plastidic localization, whereby the respective line discloses in column 7 the fine chemical heptadecanoic acid. For example, an increase of the heptadecanoic acid of at least 1 percent, particularly in a range of 14 to 35-percent is conferred as compared to a corresponding non-transformed wild type non-human organism.

Accordingly, in one embodiment, an increase of 2-hydroxypalmitic acid in a non-human organism, as compared to a corresponding non-transformed wild type non-human organism, is conferred in the process of the invention, if the activity of a polypeptide showing the activity of a b4205-protein, or if the activity of the polypeptide B4205, preferably represented by SEQ ID NO. 126976, or a hornolog or fragment thereof, or if the activity of a polypeptide encoded by a nucleic acid molecule comprising the nucleic acid SEQ ID NO. 126975, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 126975 or polypeptide SEQ ID NO. 126976, respectively, or a homolog or a fragment thereof, is increased or generated, or if the activity b4205-protein is increased or generated in a non-human organism, like a microorganism or a plant cell, plant or part thereof, especially with plastidic localization, whereby the respective line discloses in column 7 the fine chemical 2-hydroxypalmitic acid. For example, an increase of the 2-hydroxypalmitic acid of at least 1 percent, particularly in a range of 20 to 34-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a vacuolar processing enzyme, or if the activity of the polypeptide C_pp032007025r, preferably represented by SEQ ID NO. 126992, or a homolog or fragment thereof, or if the activity of a polypeptide encoded by a nucleic acid molecule comprising the nucleic acid SEQ ID NO. 126991, preferably the coding region thereof, or a homolog or fragment thereof, e.g. derived from Physcomitrella patens, is in creased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 126991 or polypeptide SEQ ID NO. 126992, respectively, or a homolog or a fragment thereof, is increased or generated, or if the activity vacuolar processing enzyme is increased or generated in a non-human organism, like a microorganism or a plant cell, plant or part thereof, especially with non-targeted localization, whereby the respective line discloses in column 7 the fine chemical 2-hydroxybehenic acid. For example, an increase of the 2-hydroxybehenic acid of at least 1 percent, particularly in a range of 31 to 52-percent is conferred as compared to a corresponding non-transformed wild type non-human organism.

Accordingly, in one embodiment, an increase of 2-hydroxypalmitic acid in a non-human organism, as compared to a corresponding non-transformed wild type non-human organism, is conferred in the process of the invention, if the activity of a polypeptide showing the activity of a glutaredoxin, or if the activity of the polypeptide GM02LC12622, preferably represented by SEQ ID NO. 10812, or a homolog or fragment thereof, or if the activity of a polypeptide encoded by a nucleic acid molecule comprising the nucleic acid SEQ ID NO. 10811, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 10811 or polypeptide SEQ ID NO. 10812, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line discloses in column 7 the fine chemical 2-hydroxypalmitic acid. For example, an increase of the 2-hydroxypalmitic acid of at least 1 percent, particularly in a range of 23 to 48-percent is conferred as compared to a corresponding non-transformed wild type non-human organism.

Accordingly, in one embodiment, an increase of cerebronic acid in a non-human organism, as compared to a corresponding non-transformed wild type non-human organism, is conferred in the process of the invention, if the activity of a polypeptide showing the activity of a transcription factor, or if the activity of the polypeptide GM02LC19289, preferably represented by SEQ ID NO. 46752, or a homolog or fragment thereof, or if the activity of a polypeptide encoded by a nucleic acid molecule comprising the nucleic acid SEQ ID NO. 46751, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 46751 or polypeptide SEQ ID NO. 46752, respectively, or a homolog or a fragment thereof, is increased or generated, or if the activity transcription factor is increased or generated in a non-human organism, like a microorganism or a plant cell, plant or part thereof, especially with non-targeted localization, whereby the respective line discloses in column 7 the fine chemical cerebronic acid. For example, an increase of the cerebronic acid of at least 1 percent, particularly in a range of 24 to 57-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a transcription factor, or if the activity of the polypeptide GM02LC19289, preferably represented by SEQ ID NO. 46752, or a homolog or fragment thereof, or if the activity of a polypeptide encoded by a nucleic acid molecule comprising the nucleic acid SEQ ID NO. 46751, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 46751 or polypeptide SEQ ID NO. 46752, respectively, or a homolog or a fragment thereof, is increased or generated, or if the activity transcription factor is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 12 to 50-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a GM02LC38418-protein, or if the activity of the polypeptide GM02LC38418, preferably represented by SEQ ID NO. 116313, or a homolog or fragment thereof, or if the activity of a polypeptide encoded by a nucleic acid molecule comprising the nucleic acid SEQ ID NO. 116312, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 116312 or polypeptide SEQ ID NO. 116313, respectively, or a homolog or a fragment thereof, is increased or generated, or if the activity GM02LC38418-protein is increased or generated in a non-human organism, like a microorganism or a plant cell, plant or part thereof, especially with non-targeted localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 22 to 46-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a sll0281-protein, or if the activity of the polypeptide Sll0281, preferably represented by SEQ ID NO. 84866, or a homolog or fragment thereof, or if the activity of a polypeptide encoded by a nucleic acid molecule comprising the nucleic acid SEQ ID NO. 84865, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 84865 or polypeptide SEQ ID NO. 84866, respectively, or a homolog or a fragment thereof, is increased or generated, or if the activity sll0281-protein is increased or generated in a non-human organism, like a microorganism or a plant cell, plant or part thereof, especially with plastidic localization, whereby the respective line discloses in column 7 the fine chemical hexadecadienoic acid. For example, an increase of the hexadecadienoic acid of at least 1 percent, particularly in a range of 50 to 62-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a bifunctional protein (phosphoribosyltransferase and regulatory protein), or if the activity of the polypeptide Sll0368, preferably represented by SEQ ID NO. 106921, or a homolog or fragment thereof, or if the activity of a polypeptide encoded by a nucleic acid molecule comprising the nucleic acid SEQ ID NO. 106920, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 106920 or polypeptide SEQ ID NO. 106921, respectively, or a homolog or a fragment thereof, is increased or generated, or if the activity bifunctional protein (phosphoribosyltransferase and regulatory protein) is increased or generated in a non-human organism, like a microorganism or a plant cell, plant or part thereof, especially with mitochondrial localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 23 to 69-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a sll0406-protein, or if the activity of the polypeptide Sll0406, preferably represented by SEQ ID NO. 127104, or a homolog or fragment thereof, or if the activity of a polypeptide encoded by a nucleic acid molecule comprising the nucleic acid SEQ ID NO. 127103, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 127103 or polypeptide SEQ ID NO. 127104, respectively, or a homolog or a fragment thereof, is increased or generated, or if the activity sll0406-protein is increased or generated in a non-human organism, like a microorganism or a plant cell, plant or part thereof, especially with non-targeted localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 25 to 43-percent is conferred as compared to a corresponding non-transformed wild type non-human organism.

Accordingly, in one embodiment, an increase of cerebronic acid in a non-human organism, as compared to a corresponding non-transformed wild type non-human organism, is conferred in the process of the invention, if the activity of a polypeptide showing the activity of a binding-protein-dependent transport systems inner membrane component, or if the activity of the polypeptide Sll0833, preferably represented by SEQ ID NO. 101708, or a homolog or fragment thereof, or if the activity of a polypeptide encoded by a nucleic acid molecule comprising the nucleic acid SEQ ID NO. 101707, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 101707 or polypeptide SEQ ID NO. 101708, respectively, or a homolog or a fragment thereof, is increased or generated, or if the activity binding-proteindependent transport systems inner membrane component is increased or generated in a non-human organism, like a microorganism or a plant cell, plant or part thereof, especially with non-targeted localization, whereby the respective line discloses in column 7 the fine chemical cerebronic acid. For example, an increase of the cerebronic acid of at least 1 percent, particularly in a range of 28 to 41-percent is conferred as compared to a corresponding non-transformed wild type non-human organism.

Accordingly, in one embodiment, an increase of cerebronic acid in a non-human organism, as compared to a corresponding non-transformed wild type non-human organism, is conferred in the process of the invention, if the activity of a polypeptide showing the activity of a phosphatase, or if the activity of the polypeptide Sll0895, preferably represented by SEQ ID NO. 127110, or a homolog or fragment thereof, or if the activity of a polypeptide encoded by a nucleic acid molecule comprising the nucleic acid SEQ ID NO. 127109, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 127109 or polypeptide SEQ ID NO. 127110, respectively, or a homolog or a fragment thereof, is increased or generated, or if the activity phosphatase is increased or generated in a non-human organism, like a microorganism or a plant cell, plant or part thereof, especially with non-targeted localization, whereby the respective line discloses in column 7 the fine chemical cerebronic acid. For example, an increase of the cerebronic acid of at least 1 percent, particularly in a range of 24 to 35-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a phosphatase, or if the activity of the polypeptide Sll0895, preferably represented by SEQ ID NO. 127110, or a homolog or fragment thereof, or if the activity of a polypeptide encoded by a nucleic acid molecule comprising the nucleic acid SEQ ID NO. 127109, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table 1, 11 or IV, application No. 12, column 5 or 8 in the respective same line as the nucleic acid molecule SEQ ID NO. 127109 or polypeptide SEQ ID NO. 127110, respectively, or a homolog or a fragment thereof, is increased or generated, or if the activity phosphatase is increased or generated in a non-human organism, like a microorganism or a plant cell, plant or part thereof, especially with non-targeted localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 22 to 40-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a carbon dioxide concentrating mechanism protein, or if the activity of the polypeptide Sll1031, preferably represented by SEQ ID NO. 52635, or a homolog or fragment thereof, or if the activity of a polypeptide encoded by a nucleic acid molecule comprising the nucleic acid SEQ ID NO. 52634, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 52634 or polypeptide SEQ ID NO. 52635, respectively, or a homolog or a fragment thereof, is increased or generated, or if the activity carbon dioxide concentrating mechanism protein is increased or generated in a non-human organism, like a microorganism or a plant cell, plant or part thereof, especially with mitochondrial localization, whereby the respective line discloses in column 7 the fine chemical hexadecadienoic acid. For example, an increase of the hexadecadienoic acid of at least 1 percent, particularly in a range of 21 to 46-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a carbon dioxide concentrating mechanism protein, or if the activity of the polypeptide Sll1031, preferably represented by SEQ ID NO. 52635, or a homolog or fragment thereof, or if the activity of a polypeptide encoded by a nucleic acid molecule comprising the nucleic acid SEQ ID NO. 52634, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 52634 or polypeptide SEQ ID NO. 52635, respectively, or a homolog or a fragment thereof, is increased or generated, or if the activity carbon dioxide concentrating mechanism protein is increased or generated in a non-human organism, like a microorganism or a plant cell, plant or part thereof, especially with mitochondrial localization, whereby the respective line discloses in column 7 the fine chemical hexadecatrienoic acid. For example, an increase of the hexadecatrienoic acid of at least 1 percent, particularly in a range of 12 to 44-percent is conferred as compared to a corresponding non-transformed wild type non-human organism.

Accordingly, in one embodiment, an increase of cerebronic acid in a non-human organism, as compared to a corresponding non-transformed wild type non-human organism, is conferred in the process of the invention, if the activity of a polypeptide showing the activity of a coproporphyrinogen III oxidase, or if the activity of the polypeptide Sll1917, preferably represented by SEQ ID NO. 11472, or a homolog or fragment thereof, or if the activity of a polypeptide encoded by a nucleic acid molecule comprising the nucleic acid SEQ ID NO. 11471, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 11471 or polypeptide SEQ ID NO. 11472, respectively, or a homolog or a fragment thereof, is increased or generated, or if the activity coproporphyrinogen III oxidase is increased or generated in a non-human organism, like a microorganism or a plant cell, plant or part thereof, especially with mitochondrial localization, whereby the respective line discloses in column 7 the fine chemical cerebronic acid. For example, an increase of the cerebronic acid of at least 1 percent, particularly in a range of 23 to 65-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a coproporphyrinogen III oxidase, or if the activity of the polypeptide Sll1917, preferably represented by SEQ ID NO. 11472, or a homolog or fragment thereof, or if the activity of a polypeptide encoded by a nucleic acid molecule comprising the nucleic acid SEQ ID NO. 11471, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 11471 or polypeptide SEQ ID NO. 11472, respectively, or a homolog or a fragment thereof, is increased or generated, or if the activity coproporphyrinogen III oxidase is increased or generated in a non-human organism, like a microorganism or a plant cell, plant or part thereof, especially with mitochondrial localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 27 to 73-percent is conferred as compared to a corresponding non-transformed wild type non-human organism.

Accordingly, in one embodiment, an increase of 2-hydroxypalmitic acid in a non-human organ-ism, as compared to a corresponding non-transformed wild type non-human organism, is conferred in the process of the invention, if the activity of a polypeptide showing the activity of a oxidoreductase, or if the activity of the polypeptide Slr0338, preferably represented by SEQ ID NO. 11991, or a homolog or fragment thereof, or if the activity of a polypeptide encoded by a nucleic acid molecule comprising the nucleic acid SEQ ID NO. 11990, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 11990 or polypeptide SEQ ID NO. 11991, respectively, or a homolog or a fragment thereof, is increased or generated, or if the activity oxidoreductase is increased or generated in a non-human organism, like a microorganism or a plant cell, plant or part thereof, especially with non-targeted localization, whereby the respective line discloses in column 7 the fine chemical 2-hydroxypalmitic acid. For example, an increase of the 2-hydroxypalmitic acid of at least 1 percent, particularly in a range of 18 to 30-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a bifunctional purine biosynthesis protein, or if the activity of the polypeptide Slr0597, preferably represented by SEQ ID NO. 56154, or a homolog or fragment thereof, or if the activity of a polypeptide encoded by a nucleic acid molecule comprising the nucleic acid SEQ ID NO. 56153, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 56153 or polypeptide SEQ ID NO. 56154, respectively, or a homolog or a fragment thereof, is increased or generated, or if the activity bifunctional purine biosynthesis protein is increased or generated in a non-human organism, like a microorganism or a plant cell, plant or part thereof, especially with plastidic localization, whereby the respective line discloses in column 7 the fine chemical hexadecatrienoic acid. For example, an increase of the hexadecatrienoic acid of at least 1 percent, particularly in a range of 21 to 26-percent is conferred as compared to a corresponding non-transformed wild type non-human organism.

Accordingly, in one embodiment, an increase of cerebronic acid in a non-human organism, as compared to a corresponding non-transformed wild type non-human organism, is conferred in the process of the invention, if the activity of a polypeptide showing the activity of a malate dehydrogenase, or if the activity of the polypeptide Slr0721, preferably represented by SEQ ID NO. 87398, or a homolog or fragment thereof, or if the activity of a polypeptide encoded by a nucleic acid molecule comprising the nucleic acid SEQ ID NO. 87397, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 87397 or polypeptide SEQ ID NO. 87398, respectively, or a homolog or a fragment thereof, is increased or generated, or if the activity malate dehydrogenase is increased or generated in a non-human organism, like a microorganism or a plant cell, plant or part thereof, especially with plastidic localization, whereby the respective line discloses in column 7 the fine chemical cerebronic acid. For example, an increase of the cerebronic acid of at least 1 percent, particularly in a range of 24 to 37-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a glycine cleavage system H protein, or if the activity of the polypeptide Slr0879, preferably represented by SEQ ID NO. 127184, or a homolog or fragment thereof, or if the activity of a polypeptide encoded by a nucleic acid molecule comprising the nucleic acid SEQ ID NO. 127183, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 127183 or polypeptide SEQ ID NO. 127184, respectively, or a homolog or a fragment thereof, is increased or generated, or if the activity glycine cleavage system H protein is increased or generated in a non-human organism, like a microorganism or a plant cell, plant or part thereof, especially with mitochondrial localization, whereby the respective line discloses in column 7 the fine chemical hexadecatrienoic acid. For example, an increase of the hexadecatrienoic acid of at least 1 percent, particularly in a range of 17 to 38-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a permease protein of ABC transporter, or if the activity of the polypeptide Slr0949, preferably represented by SEQ ID NO. 119223, or a homolog or fragment thereof, or if the activity of a polypeptide encoded by a nucleic acid molecule comprising the nucleic acid SEQ ID NO. 119222, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 119222 or polypeptide SEQ ID NO. 119223, respectively, or a homolog or a fragment thereof, is increased or generated, or if the activity permease protein of ABC transporter is increased or generated in a non-human organism, like a microorganism or a plant cell, plant or part thereof, especially with mitochondrial localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 21 to 92-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a tryptophan synthase alpha chain, or if the activity of the polypeptide Slr0966, preferably represented by SEQ ID NO. 87656, or a homolog or fragment thereof, or if the activity of a polypeptide encoded by a nucleic acid molecule comprising the nucleic acid SEQ ID NO. 87655, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 87655 or polypeptide SEQ ID NO. 87656, respectively, or a homolog or a fragment thereof, is increased or generated, or if the activity tryptophan synthase alpha chain is increased or generated in a non-human organism, like a microorganism or a plant cell, plant or part thereof, especially with plastidic localization, whereby the respective line discloses in column 7 the fine chemical hexadecadienoic acid. For example, an increase of the hexadecadienoic acid of at least 1 percent, particularly in a range of 35 to 194-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a dihydrolipoamide dehydrogenase, or if the activity of the polypeptide Slr1096, preferably represented by SEQ ID NO. 57735, or a homolog or fragment thereof, or if the activity of a polypeptide encoded by a nucleic acid molecule comprising the nucleic acid SEQ ID NO. 57734, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 57734 or polypeptide SEQ ID NO. 57735, respectively, or a homolog or a fragment thereof, is increased or generated, or if the activity dihydrolipoamide dehydrogenase is increased or generated in a non-human organism, like a microorganism or a plant cell, plant or part thereof, especially with non-targeted localization, whereby the respective line discloses in column 7 the fine chemical 2-hydroxybehenic acid. For example, an increase of the 2-hydroxybehenic acid of at least 1 percent, particularly in a range of 31 to 55-percent is conferred as compared to a corresponding non-transformed wild type non-human organism.

Accordingly, in one embodiment, an increase of 2-hydroxypalmitic acid in a non-human organism, as compared to a corresponding non-transformed wild type non-human organism, is conferred in the process of the invention, if the activity of a polypeptide showing the activity of a phosphate import ATP-binding protein, or if the activity of the polypeptide Slr1250, preferably represented by SEQ ID NO. 116461, or a homolog or fragment thereof, or if the activity of a polypeptide encoded by a nucleic acid molecule comprising the nucleic acid SEQ ID NO. 116460, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 116460 or polypeptide SEQ ID NO. 116461, respectively, or a homolog or a fragment thereof, is increased or generated, or if the activity phosphate import ATP-binding protein is increased or generated in a non-human organism, like a microorganism or a plant cell, plant or part thereof, especially with mitochondrial localization, whereby the respective line discloses in column 7 the fine chemical 2-hydroxypalmitic acid. For example, an increase of the 2-hydroxypalmitic acid of at least 1 percent, particularly in a range of 19 to 38-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a glutamate 5-kinase, or if the activity of the polypeptide Slr2035, preferably represented by SEQ ID NO. 127522, or a homolog or fragment thereof, or if the activity of a polypeptide encoded by a nucleic acid molecule comprising the nucleic acid SEQ ID NO. 127521, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 127521 or polypeptide SEQ ID NO. 127522, respectively, or a homolog or a fragment thereof, is increased or generated, or if the activity glutamate 5-kinase is increased or generated in a non-human organism, like a microorganism or a plant cell, plant or part thereof, especially with mitochondrial localization, whereby the respective line discloses in column 7 the fine chemical hexadecatrienoic acid. For example, an increase of the hexadecatrienoic acid of at least 1 percent, particularly in a range of 12 to 23-percent is conferred as compared to a corresponding non-transformed wild type non-human organism.

Accordingly, in one embodiment, an increase of cerebronic acid in a non-human organism, as compared to a corresponding non-transformed wild type non-human organism, is conferred in the process of the invention, if the activity of a polypeptide showing the activity of a threonine dehydratase, or if the activity of the polypeptide Slr2072, preferably represented by SEQ ID NO. 12342, or a homolog or fragment thereof, or if the activity of a polypeptide encoded by a nucleic acid molecule comprising the nucleic acid SEQ ID NO. 12341, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 12341 or polypeptide SEQ ID NO. 12342, respectively, or a homolog or a fragment thereof, is increased or generated, or if the activity threonine dehydratase is increased or generated in a non-human organism, like a microorganism or a plant cell, plant or part thereof, especially with mitochondrial localization, whereby the respective line discloses in column 7 the fine chemical cerebronic acid. For example, an increase of the cerebronic acid of at least 1 percent, particularly in a range of 30 to 42-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a threonine dehydratase, or if the activity of the polypeptide Slr2072, preferably represented by SEQ ID NO. 12342, or a homolog or fragment thereof, or if the activity of a polypeptide encoded by a nucleic acid molecule comprising the nucleic acid SEQ ID NO. 12341, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 12341 or polypeptide SEQ ID NO. 12342, respectively, or a homolog or a fragment thereof, is increased or generated, or if the activity threonine dehydratase is increased or generated in a non-human organism, like a microorganism or a plant cell, plant or part thereof, especially with mitochondrial localization, whereby the respective line discloses in column 7 the fine chemical hexadecatrienoic acid. For example, an increase of the hexadecatrienoic acid of at least 1 percent, particularly in a range of 15 to 59-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a threonine dehydratase, or if the activity of the polypeptide Slr2072, preferably represented by SEQ ID NO. 12342, or a homolog or fragment thereof, or if the activity of a polypeptide encoded by a nucleic acid molecule comprising the nucleic acid SEQ ID NO. 12341, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 12341 or polypeptide SEQ ID NO. 12342, respectively, or a homolog or a fragment thereof, is increased or generated, or if the activity threonine dehydratase is increased or generated in a non-human organism, like a microorganism or a plant cell, plant or part thereof, especially with plastidic localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 27 to 38-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a ferredoxin, or if the activity of the polypeptide Ssl3044, preferably represented by SEQ ID NO. 128025, or a homolog or fragment thereof, or if the activity of a polypeptide encoded by a nucleic acid molecule comprising the nucleic acid SEQ ID NO. 128024, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 128024 or polypeptide SEQ ID NO. 128025, respectively, or a homolog or a fragment thereof, is increased or generated, or if the activity ferredoxin is increased or generated in a non-human organism, like a microorganism or a plant cell, plant or part thereof, especially with plastidic localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 22 to 37-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a Sec-independent protein translocase subunit, or if the activity of the polypeptide TTC0019, preferably represented by SEQ ID NO. 12699, or a homolog or fragment thereof, or if the activity of a polypeptide encoded by a nucleic acid molecule comprising the nucleic acid SEQ ID NO. 12698, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 12698 or polypeptide SEQ ID NO. 12699, respectively, or a homolog or a fragment thereof, is increased or generated, or if the activity Sec-independent protein translocase subunit is increased or generated in a non-human organism, like a microorganism or a plant cell, plant or part thereof, especially with non-targeted localization, whereby the respective line discloses in column 7 the fine chemical 2-hydroxybehenic acid. For example, an increase of the 2-hydroxybehenic acid of at least 1 percent, particularly in a range of 31 to 39-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a amino acid ABC transporter permease protein, or if the activity of the polypeptide TTC0337, preferably represented by SEQ ID NO. 61071, or a homolog or fragment thereof, or if the activity of a polypeptide encoded by a nucleic acid molecule comprising the nucleic acid SEQ ID NO. 61070, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 61070 or polypeptide SEQ ID NO. 61071, respectively, or a homolog or a fragment thereof, is increased or generated, or if the activity amino acid ABC transporter permease protein is increased or generated in a non-human organism, like a microorganism or a plant cell, plant or part thereof, especially with non-targeted localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 30 to 53-percent is conferred as compared to a corresponding non-transformed wild type non-human organism.

Accordingly, in one embodiment, an increase of cerebronic acid in a non-human organism, as compared to a corresponding non-transformed wild type non-human organism, is conferred in the process of the invention, if the activity of a polypeptide showing the activity of a metal-dependent hydrolase, or if the activity of the polypeptide TTC0917, preferably represented by SEQ ID NO. 61554, or a homolog or fragment thereof, or if the activity of a polypeptide encoded by a nucleic acid molecule comprising the nucleic acid SEQ ID NO. 61553, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 61553 or polypeptide SEQ ID NO. 61554, respectively, or a homolog or a fragment thereof, is increased or generated, or if the activity metal-dependent hydrolase is increased or generated in a non-human organism, like a microorganism or a plant cell, plant or part thereof, especially with non-targeted localization, whereby the respective line discloses in column 7 the fine chemical cerebronic acid. For example, an increase of the cerebronic acid of at least 1 percent, particularly in a range of 27 to 89-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a metal-dependent hydrolase, or if the activity of the polypeptide TTC0917, preferably represented by SEQ ID NO. 61554, or a homolog or fragment thereof, or if the activity of a polypeptide encoded by a nucleic acid molecule comprising the nucleic acid SEQ ID NO. 61553, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 61553 or polypeptide SEQ ID NO. 61554, respectively, or a homolog or a fragment thereof, is increased or generated, or if the activity metal-dependent hydrolase is increased or generated in a non-human organism, like a microorganism or a plant cell, plant or part thereof, especially with non-targeted localization, whereby the respective line discloses in column 7 the fine chemical hexadecadienoic acid. For example, an increase of the hexadecadienoic acid of at least 1 percent, particularly in a range of 22 to 139-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a multiple antibiotic resistance protein, or if the activity of the polypeptide TTC1193, preferably represented by SEQ ID NO. 61724, or a homolog or fragment thereof, or if the activity of a polypeptide encoded by a nucleic acid molecule comprising the nucleic acid SEQ ID NO. 61723, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 61723 or polypeptide SEQ ID NO. 61724, respectively, or a homolog or a fragment thereof, is increased or generated, or if the activity multiple antibiotic resistance protein is increased or generated in a non-human organism, like a microorganism or a plant cell, plant or part thereof, especially with non-targeted localization, whereby the respective line discloses in column 7 the fine chemical 2-hydroxybehenic acid. For example, an increase of the 2-hydroxybehenic acid of at least 1 percent, particularly in a range of 28 to 35-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a TTC1386-protein, or if the activity of the polypeptide TTC1386, preferably represented by SEQ ID NO. 62080, or a homolog or fragment thereof, or if the activity of a polypeptide encoded by a nucleic acid molecule comprising the nucleic acid SEQ ID NO. 62079, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 62079 or polypeptide SEQ ID NO. 62080, respectively, or a homolog or a fragment thereof, is increased or generated, or if the activity TTC1386-protein is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 14 to 26-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a homocitrate synthase, or if the activity of the polypeptide TTC1550, preferably represented by SEQ ID NO. 12975, or a homolog or fragment thereof, or if the activity of a polypeptide encoded by a nucleic acid molecule comprising the nucleic acid SEQ ID NO. 12974, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 12974 or polypeptide SEQ ID NO. 12975, respectively, or a homolog or a fragment thereof, is increased or generated, or if the activity homocitrate synthase is increased or generated in a non-human organism, like a microorganism or a plant cell, plant or part thereof, especially with non-targeted localization, whereby the respective line discloses in column 7 the fine chemical 2-hydroxybehenic acid. For example, an increase of the 2-hydroxybehenic acid of at least 1 percent, particularly in a range of 26 to 57-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a protein kinase, or if the activity of the polypeptide Yar019c, preferably represented by SEQ ID NO. 128140, or a homolog or fragment thereof, or if the activity of a polypeptide encoded by a nucleic acid molecule comprising the nucleic acid SEQ ID NO. 128139, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 128139 or polypeptide SEQ ID NO. 128140, respectively, or a homolog or a fragment thereof, is increased or generated, or if the activity protein kinase is increased or generated in a non-human organism, like a microorganism or a plant cell, plant or part thereof, especially with non-targeted localization, whereby the respective line discloses in column 7 the fine chemical hexadecadienoic acid. For example, an increase of the hexadecadienoic acid of at least 1 percent, particularly in a range of 21 to 25-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a protein kinase, or if the activity of the polypeptide Yar019c, preferably represented by SEQ ID NO. 128140, or a homolog or fragment thereof, or if the activity of a polypeptide encoded by a nucleic acid molecule comprising the nucleic acid SEQ ID NO. 128139, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 128139 or polypeptide SEQ ID NO. 128140, respectively, or a homolog or a fragment thereof, is increased or generated, or if the activity protein kinase is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 13 to 20-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a transcription factor, or if the activity of the polypeptide Ybl021c, preferably represented by SEQ ID NO. 62525, or a homolog or fragment thereof, or if the activity of a polypeptide encoded by a nucleic acid molecule comprising the nucleic acid SEQ ID NO. 62524, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 62524 or polypeptide SEQ ID NO. 62525, respectively, or a homolog or a fragment thereof, is increased or generated, or if the activity transcription factor is increased or generated in a non-human organism, like a microorganism or a plant cell, plant or part thereof, especially with non-targeted localization, whereby the respective line discloses in column 7 the fine chemical hexadecadienoic acid. For example, an increase of the hexadecadienoic acid of at least 1 percent, particularly in a range of 21 to 96-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a transcription factor, or if the activity of the polypeptide Ybl021c, preferably represented by SEQ ID NO. 62525, or a homolog or fragment thereof, or if the activity of a polypeptide encoded by a nucleic acid molecule comprising the nucleic acid SEQ ID NO. 62524, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 62524 or polypeptide SEQ ID NO. 62525, respectively, or a homolog or a fragment thereof, is increased or generated, or if the activity transcription factor is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 12 to 23-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a cell division control protein, or if the activity of the polypeptide Ybr160w, preferably represented by SEQ ID NO. 62718, or a homolog or fragment thereof, or if the activity of a polypeptide encoded by a nucleic acid molecule comprising the nucleic acid SEQ ID NO. 62717, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 62717 or polypeptide SEQ ID NO. 62718, respectively, or a homolog or a fragment thereof, is increased or generated, or if the activity cell division control protein is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 13 to 54-percent is conferred as compared to a corresponding non-transformed wild type non-human organism.

Accordingly, in one embodiment, an increase of cerebronic acid in a non-human organism, as compared to a corresponding non-transformed wild type non-human organism, is conferred in the process of the invention, if the activity of a polypeptide showing the activity of a ycl027w-protein, or if the activity of the polypeptide Ycl027w, preferably represented by SEQ ID NO. 96981, or a homolog or fragment thereof, or if the activity of a polypeptide encoded by a nucleic acid molecule comprising the nucleic acid SEQ ID NO. 96980, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 96980 or polypeptide SEQ ID NO. 96981, respectively, or a homolog or a fragment thereof, is increased or generated, or if the activity ycl027w-protein is increased or generated in a non-human organism, like a microorganism or a plant cell, plant or part thereof, especially with plastidic localization, whereby the respective line discloses in column 7 the fine chemical cerebronic acid. For example, an increase of the cerebronic acid of at least 1 percent, particularly in a range of 25 to 46-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a ycl027w-protein, or if the activity of the polypeptide Ycl027w, preferably represented by SEQ ID NO. 96981, or a homolog or fragment thereof, or if the activity of a polypeptide encoded by a nucleic acid molecule comprising the nucleic acid SEQ ID NO. 96980, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 96980 or polypeptide SEQ ID NO. 96981, respectively, or a homolog or a fragment thereof, is increased or generated, or if the activity ycl027w-protein is increased or generated in a non-human organism, like a microorganism or a plant cell, plant or part thereof, especially with plastidic localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 25 to 41-percent is conferred as compared to a corresponding non-transformed wild type non-human organism.

Accordingly, in one embodiment, an increase of 2-hydroxypalmitic acid in a non-human organism, as compared to a corresponding non-transformed wild type non-human organism, is conferred in the process of the invention, if the activity of a polypeptide showing the activity of a ycl049c-protein, or if the activity of the polypeptide Ycl049c, preferably represented by SEQ ID NO. 113221, or a homolog or fragment thereof, or if the activity of a polypeptide encoded by a nucleic acid molecule comprising the nucleic acid SEQ ID NO. 113220, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 113220 or polypeptide SEQ ID NO. 113221, respectively, or a homolog or a fragment thereof, is increased or generated, or if the activity ycl049c-protein is increased or generated in a non-human organism, like a microorganism or a plant cell, plant or part thereof, especially with plastidic localization, whereby the respective line discloses in column 7 the fine chemical 2-hydroxypalmitic acid. For example, an increase of the 2-hydroxypalmitic acid of at least 1 percent, particularly in a range of 22 to 74-percent is conferred as compared to a corresponding non-transformed wild type non-human organism.

Accordingly, in one embodiment, an increase of oleic acid in a non-human organism, as compared to a corresponding non-transformed wild type non-human organism, is conferred in the process of the invention, if the activity of a polypeptide showing the activity of a glycerol-3-phosphate dehydrogenase, or if the activity of the polypeptide Ydl022w, preferably represented by SEQ ID NO. 119409, or a homolog or fragment thereof, or if the activity of a polypeptide encoded by a nucleic acid molecule comprising the nucleic acid SEQ ID NO. 119408, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 119408 or polypeptide SEQ ID NO. 119409, respectively, or a homolog or a fragment thereof, is increased or generated, or if the activity glycerol-3-phosphate dehydrogenase is increased or generated in a non-human organism, like a microorganism or a plant cell, plant or part thereof, especially with non-targeted localization, whereby the respective line discloses in column 7 the fine chemical oleic acid. For example, an increase of the oleic acid of at least 1 percent, particularly in a range of 34 to 156-percent is conferred as compared to a corresponding non-transformed wild type non-human organism.

Accordingly, in one embodiment, an increase of eicosanoic acid in a non-human organism, as compared to a corresponding non-transformed wild type non-human organism, is conferred in the process of the invention, if the activity of a polypeptide showing the activity of a isocitrate dehydrogenase, or if the activity of the polypeptide Ydl066w, preferably represented by SEQ ID NO. 122229, or a homolog or fragment thereof, or if the activity of a polypeptide encoded by a nucleic acid molecule comprising the nucleic acid SEQ ID NO. 122228, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 122228 or polypeptide SEQ ID NO. 122229, respectively, or a homolog or a fragment thereof, is increased or generated, or if the activity isocitrate dehydrogenase is increased or generated in a non-human organism, like a microorganism or a plant cell, plant or part thereof, especially with non-targeted localization, whereby the respective line discloses in column 7 the fine chemical eicosanoic acid. For example, an increase of the eicosanoic acid of at least 1 percent, particularly in a range of 8 to 17-percent is conferred as compared to a corresponding non-transformed wild type non-human organism.

Accordingly, in one embodiment, an increase of heptadecanoic acid in a non-human organism, as compared to a corresponding non-transformed wild type non-human organism, is conferred in the process of the invention, if the activity of a polypeptide showing the activity of a isocitrate dehydrogenase, or if the activity of the polypeptide Ydl066w, preferably represented by SEQ ID NO. 122229, or a homolog or fragment thereof, or if the activity of a polypeptide encoded by a nucleic acid molecule comprising the nucleic acid SEQ ID NO. 122228, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 122228 or polypeptide SEQ ID NO. 122229, respectively, or a homolog or a fragment thereof, is increased or generated, or if the activity isocitrate dehydrogenase is increased or generated in a non-human organism, like a microorganism or a plant cell, plant or part thereof, especially with non-targeted localization, whereby the respective line discloses in column 7 the fine chemical heptadecanoic acid. For example, an increase of the heptadecanoic acid of at least 1 percent, particularly in a range of 16 to 20-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a cell division control protein, or if the activity of the polypeptide Ydl126c, preferably represented by SEQ ID NO. 120046, or a homolog or fragment thereof, or if the activity of a polypeptide encoded by a nucleic acid molecule comprising the nucleic acid SEQ ID NO. 120045, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 120045 or polypeptide SEQ ID NO. 120046, respectively, or a homolog or a fragment thereof, is increased or generated, or if the activity cell division control protein is increased or generated in a non-human organism, like a microorganism or a plant cell, plant or part thereof, especially with non-targeted localization, whereby the respective line discloses in column 7 the fine chemical hexadecadienoic acid. For example, an increase of the hexadecadienoic acid of at least 1 percent, particularly in a range of 30 to 63-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a cell division control protein, or if the activity of the polypeptide Ydl126c, preferably represented by SEQ ID NO. 120046, or a homolog or fragment thereof, or if the activity of a polypeptide encoded by a nucleic acid molecule comprising the nucleic acid SEQ ID NO. 120045, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 120045 or polypeptide SEQ ID NO. 120046, respectively, or a homolog or a fragment thereof, is increased or generated, or if the activity cell division control protein is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 14 to 34-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a homocitrate synthase, or if the activity of the polypeptide Ydl131w, preferably represented by SEQ ID NO. 113225, or a homolog or fragment thereof, or if the activity of a polypeptide encoded by a nucleic acid molecule comprising the nucleic acid SEQ ID NO. 113224, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 113224 or polypeptide SEQ ID NO. 113225, respectively, or a homolog or a fragment thereof, is increased or generated, or if the activity homocitrate synthase is increased or generated in a non-human organism, like a microorganism or a plant cell, plant or part thereof, especially with plastidic localization, whereby the respective line discloses in column 7 the fine chemical 2-hydroxybehenic acid. For example, an increase of the 2-hydroxybehenic acid of at least 1 percent, particularly in a range of 29 to 50-percent is conferred as compared to a corresponding non-transformed wild type non-human organism.

Accordingly, in one embodiment, an increase of behenic acid in a non-human organism, as compared to a corresponding non-transformed wild type non-human organism, is conferred in the process of the invention, if the activity of a polypeptide showing the activity of a phospho-2-dehydro-3-deoxyheptonate aldolase, or if the activity of the polypeptide Ydr035w, preferably represented by SEQ ID NO. 113331, or a homolog or fragment thereof, or if the activity of a polypeptide encoded by a nucleic acid molecule comprising the nucleic acid SEQ ID NO. 113330, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 113330 or polypeptide SEQ ID NO. 113331, respectively, or a homolog or a fragment thereof, is increased or generated, or if the activity phospho-2-dehydro-3-deoxyheptonate aldolase is increased or generated in a non-human organism, like a microorganism or a plant cell, plant or part thereof, especially with plastidic localization, whereby the respective line discloses in column 7 the fine chemical behenic acid. For example, an increase of the behenic acid of at least 1 percent, particularly in a range of 38 to 128-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a cell wall protein, or if the activity of the polypeptide Ydr077w, preferably represented by SEQ ID NO. 128149, or a homolog or fragment thereof, or if the activity of a polypeptide encoded by a nucleic acid molecule comprising the nucleic acid SEQ ID NO. 128148, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 128148 or polypeptide SEQ ID NO. 128149, respectively, or a homolog or a fragment thereof, is increased or generated, or if the activity cell wall protein is increased or generated in a non-human organism, like a microorganism or a plant cell, plant or part thereof, especially with plastidic localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 22 to 32-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a F-box protein, or if the activity of the polypeptide Ydr131c, preferably represented by SEQ ID NO. 14707, or a homolog or fragment thereof, or if the activity of a polypeptide encoded by a nucleic acid molecule comprising the nucleic acid SEQ ID NO. 14706, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 14706 or polypeptide SEQ ID NO. 14707, respectively, or a homolog or a fragment thereof, is increased or generated, or if the activity F-box protein is increased or generated in a non-human organism, like a microorganism or a plant cell, plant or part thereof, especially with plastidic localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 26 to 66-percent is conferred as compared to a corresponding non-transformed wild type non-human organism.

Accordingly, in one embodiment, an increase of cerebronic acid in a non-human organism, as compared to a corresponding non-transformed wild type non-human organism, is conferred in the process of the invention, if the activity of a polypeptide showing the activity of a ydr273w-protein, or if the activity of the polypeptide Ydr273w, preferably represented by SEQ ID NO. 90160, or a homolog or fragment thereof, or if the activity of a polypeptide encoded by a nucleic acid molecule comprising the nucleic acid SEQ ID NO. 90159, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 90159 or polypeptide SEQ ID NO. 90160, respectively, or a homolog or a fragment thereof, is increased or generated, or if the activity ydr273w-protein is increased or generated in a non-human organism, like a microorganism or a plant cell, plant or part thereof, especially with plastidic localization, whereby the respective line discloses in column 7 the fine chemical cerebronic acid. For example, an increase of the cerebronic acid of at least 1 percent, particularly in a range of 27 to 30-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a ydr273w-protein, or if the activity of the polypeptide Ydr273w, preferably represented by SEQ ID NO. 90160, or a homolog or fragment thereof, or if the activity of a polypeptide encoded by a nucleic acid molecule comprising the nucleic acid SEQ ID NO. 90159, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 90159 or polypeptide SEQ ID NO. 90160, respectively, or a homolog or a fragment thereof, is increased or generated, or if the activity ydr273w-protein is increased or generated in a non-human organism, like a microorganism or a plant cell, plant or part thereof, especially with plastidic localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 24 to 28-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a ydr339c-protein, or if the activity of the polypeptide Ydr339c, preferably represented by SEQ ID NO. 120242, or a homolog or fragment thereof, or if the activity of a polypeptide encoded by a nucleic acid molecule comprising the nucleic acid SEQ ID NO. 120241, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 120241 or polypeptide SEQ ID NO. 120242, respectively, or a homolog or a fragment thereof, is increased or generated, or if the activity ydr339c-protein is increased or generated in a non-human organism, like a microorganism or a plant cell, plant or part thereof, especially with plastidic localization, whereby the respective line discloses in column 7 the fine chemical hexadecatrienoic acid. For example, an increase of the hexadecatrienoic acid of at least 1 percent, particularly in a range of 14 to 20-percent is conferred as compared to a corresponding non-transformed wild type non-human organism.

Accordingly, in one embodiment, an increase of heptadecanoic acid in a non-human organism, as compared to a corresponding non-transformed wild type non-human organism, is conferred in the process of the invention, if the activity of a polypeptide showing the activity of a aldolase, or if the activity of the polypeptide Yel046c, preferably represented by SEQ ID NO. 128512, or a homolog or fragment thereof, or if the activity of a polypeptide encoded by a nucleic acid molecule comprising the nucleic acid SEQ ID NO. 128511, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 128511 or polypeptide SEQ ID NO. 128512, respectively, or a homolog or a fragment thereof, is increased or generated, or if the activity aldolase is increased or generated in a non-human organism, like a microorganism or a plant cell, plant or part thereof, especially with plastidic localization, whereby the respective line discloses in column 7 the fine chemical heptadecanoic acid. For example, an increase of the heptadecanoic acid of at least 1 percent, particularly in a range of 20 to 35-percent is conferred as compared to a corresponding non-transformed wild type non-human organism.

Accordingly, in one embodiment, an increase of myristic acid in a non-human organism, as compared to a corresponding non-transformed wild type non-human organism, is conferred in the process of the invention, if the activity of a polypeptide showing the activity of a small nuclear ribonucleoprotein , or if the activity of the polypeptide Yer112w, preferably represented by SEQ ID NO. 113599, or a homolog or fragment thereof, or if the activity of a polypeptide encoded by a nucleic acid molecule comprising the nucleic acid SEQ ID NO. 113598, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 113598 or polypeptide SEQ ID NO. 113599, respectively, or a homolog or a fragment thereof, is increased or generated, or if the activity small nuclear ribonucleoprotein is increased or generated in a non-human organism, like a microorganism or a plant cell, plant or part thereof, especially with non-targeted localization, whereby the respective line discloses in column 7 the fine chemical myristic acid. For example, an increase of the myristic acid of at least 1 percent, particularly in a range of 46 to 90-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a molecular chaperone portein, or if the activity of the polypeptide Yfl016c, preferably represented by SEQ ID NO. 63808, or a homolog or fragment thereof, or if the activity of a polypeptide encoded by a nucleic acid molecule comprising the nucleic acid SEQ ID NO. 63807, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 63807 or polypeptide SEQ ID NO. 63808, respectively, or a homolog or a fragment thereof, is increased or generated, or if the activity molecular chaperone portein is increased or generated in a non-human organism, like a microorganism or a plant cell, plant or part thereof, especially with non-targeted localization, whereby the respective line discloses in column 7 the fine chemical hexadecadienoic acid. For example, an increase of the hexadecadienoic acid of at least 1 percent, particularly in a range of 20 to 48-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a molecular chaperone portein, or if the activity of the polypeptide Yfl016c, preferably represented by SEQ ID NO. 63808, or a homolog or fragment thereof, or if the activity of a polypeptide encoded by a nucleic acid molecule comprising the nucleic acid SEQ ID NO. 63807, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 63807 or polypeptide SEQ ID NO. 63808, respectively, or a homolog or a fragment thereof, is increased or generated, or if the activity molecular chaperone portein is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 13 to 39-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a YFL019C-protein, or if the activity of the polypeptide Yfl019c, preferably represented by SEQ ID NO. 64145, or a homolog or fragment thereof, or if the activity of a polypeptide encoded by a nucleic acid molecule comprising the nucleic acid SEQ ID NO. 64144, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 64144 or polypeptide SEQ ID NO. 64145, respectively, or a homolog or a fragment thereof, is increased or generated, or if the activity YFL019C-protein is increased or generated in a non-human organism, like a microorganism or a plant cell, plant or part thereof, especially with non-targeted localization, whereby the respective line discloses in column 7 the fine chemical 2-hydroxybehenic acid. For example, an increase of the 2-hydroxybehenic acid of at least 1 percent, particularly in a range of 31 to 108-percent is conferred as compared to a corresponding non-transformed wild type non-human organism.

Accordingly, in one embodiment, an increase of 2-hydroxypalmitic acid in a non-human organism, as compared to a corresponding non-transformed wild type non-human organism, is conferred in the process of the invention, if the activity of a polypeptide showing the activity of a YFL019C-protein, or if the activity of the polypeptide Yfl019c, preferably represented by SEQ ID NO. 64145, or a homolog or fragment thereof, or if the activity of a polypeptide encoded by a nucleic acid molecule comprising the nucleic acid SEQ ID NO. 64144, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 64144 or polypeptide SEQ ID NO. 64145, respectively, or a homolog or a fragment thereof, is increased or generated, or if the activity YFL019C-protein is increased or generated in a non-human organism, like a microorganism or a plant cell, plant or part thereof, especially with non-targeted localization, whereby the respective line discloses in column 7 the fine chemical 2-hydroxypalmitic acid. For example, an increase of the 2-hydroxypalmitic acid of at least 1 percent, particularly in a range of 21 to 75-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a aminotransferase, or if the activity of the polypeptide Yfl030w, preferably represented by SEQ ID NO. 90190, or a homolog or fragment thereof, or if the activity of a polypeptide encoded by a nucleic acid molecule comprising the nucleic acid SEQ ID NO. 90189, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 90189 or polypeptide SEQ ID NO. 90190, respectively, or a homolog or a fragment thereof, is increased or generated, or if the activity aminotransferase is increased or generated in a non-human organism, like a microorganism or a plant cell, plant or part thereof, especially with plastidic localization, whereby the respective line discloses in column 7 the fine chemical hexadecadienoic acid. For example, an increase of the hexadecadienoic acid of at least 1 percent, particularly in a range of 31 to 53-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a ygl237c-protein, or if the activity of the polypeptide Ygl237c, preferably represented by SEQ ID NO. 64178, or a homolog or fragment thereof, or if the activity of a polypeptide encoded by a nucleic acid molecule comprising the nucleic acid SEQ ID NO. 64177, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 64177 or polypeptide SEQ ID NO. 64178, respectively, or a homolog or a fragment thereof, is increased or generated, or if the activity ygl237c-protein is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 13 to 35-percent is conferred as compared to a corresponding non-transformed wild type non-human organism.

Accordingly, in one embodiment, an increase of eicosanoic acid in a non-human organism, as compared to a corresponding non-transformed wild type non-human organism, is conferred in the process of the invention, if the activity of a polypeptide showing the activity of a YGR104C-protein, or if the activity of the polypeptide Ygr104c, preferably represented by SEQ ID NO. 117483, or a homolog or fragment thereof, or if the activity of a polypeptide encoded by a nucleic acid molecule comprising the nucleic acid SEQ ID NO. 117482, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 117482 or polypeptide SEQ ID NO. 117483, respectively, or a homolog or a fragment thereof, is increased or generated, or if the activity YGR1040-protein is increased or generated in a non-human organism, like a microorganism or a plant cell, plant or part thereof, especially with non-targeted localization, whereby the respective line discloses in column 7 the fine chemical eicosanoic acid. For example, an increase of the eicosanoic acid of at least 1 percent, particularly in a range of 11 to 19-percent is conferred as compared to a corresponding non-transformed wild type non-human organism.

Accordingly, in one embodiment, an increase of cerebronic acid in a non-human organism, as compared to a corresponding non-transformed wild type non-human organism, is conferred in the process of the invention, if the activity of a polypeptide showing the activity of a squalene monooxygenase, or if the activity of the polypeptide Ygr175c, preferably represented by SEQ ID NO. 64219, or a homolog or fragment thereof, or if the activity of a polypeptide encoded by a nucleic acid molecule comprising the nucleic acid SEQ ID NO. 64218, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 64218 or polypeptide SEQ ID NO. 64219, respectively, or a homolog or a fragment thereof, is increased or generated, or if the activity squalene monooxygenase is increased or generated in a non-human organism, like a microorganism or a plant cell, plant or part thereof, especially with plastidic localization, whereby the respective line discloses in column 7 the fine chemical cerebronic acid. For example, an increase of the cerebronic acid of at least 1 percent, particularly in a range of 24 to 34-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a biotin synthase, or if the activity of the polypeptide Ygr286c, preferably represented by SEQ ID NO. 128656, or a homolog or fragment thereof, or if the activity of a polypeptide encoded by a nucleic acid molecule comprising the nucleic acid SEQ ID NO. 128655, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 128655 or polypeptide SEQ ID NO. 128656, respectively, or a homolog or a fragment thereof, is increased or generated, or if the activity biotin synthase is increased or generated in a non-human organism, like a microorganism or a plant cell, plant or part thereof, especially with plastidic localization, whereby the respective line discloses in column 7 the fine chemical hexadecadienoic acid. For example, an increase of the hexadecadienoic acid of at least 1 percent, particularly in a range of 23 to 46-percent is conferred as compared to a corresponding non-transformed wild type non-human organism.

Accordingly, in one embodiment, an increase of cerebronic acid in a non-human organism, as compared to a corresponding non-transformed wild type non-human organism, is conferred in the process of the invention, if the activity of a polypeptide showing the activity of a yhr033w-protein, or if the activity of the polypeptide Yhr033w, preferably represented by SEQ ID NO. 128968, or a homolog or fragment thereof, or if the activity of a polypeptide encoded by a nucleic acid molecule comprising the nucleic acid SEQ ID NO. 128967, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 128967 or polypeptide SEQ ID NO. 128968, respectively, or a homolog or a fragment thereof, is increased or generated, or if the activity yhr033w-protein is increased or generated in a non-human organism, like a microorganism or a plant cell, plant or part thereof, especially with plastidic localization, whereby the respective line discloses in column 7 the fine chemical cerebronic acid. For example, an increase of the cerebronic acid of at least 1 percent, particularly in a range of 21 to 27-percent is conferred as compared to a corresponding non-transformed wild type non-human organism.

Accordingly, in one embodiment, an increase of cerebronic acid in a non-human organism, as compared to a corresponding non-transformed wild type non-human organism, is conferred in the process of the invention, if the activity of a polypeptide showing the activity of a DnaJ-like chaperone, or if the activity of the polypeptide Yjl073w, preferably represented by SEQ ID NO. 64965, or a homolog or fragment thereof, or if the activity of a polypeptide encoded by a nucleic acid molecule comprising the nucleic acid SEQ ID NO. 64964, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 64964 or polypeptide SEQ ID NO. 64965, respectively, or a homolog or a fragment thereof, is increased or generated, or if the activity DnaJ-like chaperone is increased or generated in a non-human organism, like a microorganism or a plant cell, plant or part thereof, especially with plastidic localization, whereby the respective line discloses in column 7 the fine chemical cerebronic acid. For example, an increase of the cerebronic acid of at least 1 percent, particularly in a range of 30 to 45-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a YJL127W-A-protein, or if the activity of the polypeptide Yjl127w-a, preferably represented by SEQ ID NO. 97632, or a homolog or fragment thereof, or if the activity of a polypeptide encoded by a nucleic acid molecule comprising the nucleic acid SEQ ID NO. 97631, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 97631 or polypeptide SEQ ID NO. 97632, respectively, or a homolog or a fragment thereof, is increased or generated, or if the activity YJL127W-A-protein is increased or generated in a non-human organism, like a microorganism or a plant cell, plant or part thereof, especially with non-targeted localization, whereby the respective line discloses in column 7 the fine chemical 2-hydroxybehenic acid. For example, an increase of the 2-hydroxybehenic acid of at least 1 percent, particularly in a range of 26 to 40-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a cystathionine gamma-synthase, or if the activity of the polypeptide Yjr130c, preferably represented by SEQ ID NO. 14844, or a homolog or fragment thereof, or if the activity of a polypeptide encoded by a nucleic acid molecule comprising the nucleic acid SEQ ID NO. 14843, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 14843 or polypeptide SEQ ID NO. 14844, respectively, or a homolog or a fragment thereof, is increased or generated, or if the activity cystathionine gamma-synthase is increased or generated in a non-human organism, like a microorganism or a plant cell, plant or part thereof, especially with non-targeted localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 23 to 70-percent is conferred as compared to a corresponding non-transformed wild type non-human organism.

Accordingly, in one embodiment, an increase of 2-hydroxypalmitic acid in a non-human organism, as compared to a corresponding non-transformed wild type non-human organism, is conferred in the process of the invention, if the activity of a polypeptide showing the activity of a cystathionine gamma-synthase, or if the activity of the polypeptide Yjr130c, preferably represented by SEQ ID NO. 14844, or a homolog or fragment thereof, or if the activity of a polypeptide encoded by a nucleic acid molecule comprising the nucleic acid SEQ ID NO. 14843, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 14843 or polypeptide SEQ ID NO. 14844, respectively, or a homolog or a fragment thereof, is increased or generated, or if the activity cystathionine gamma-synthase is increased or generated in a non-human organism, like a microorganism or a plant cell, plant or part thereof, especially with non-targeted localization, whereby the respective line discloses in column 7 the fine chemical 2-hydroxypalmitic acid. For example, an increase of the 2-hydroxypalmitic acid of at least 1 percent, particularly in a range of 19 to 106-percent is conferred as compared to a corresponding non-transformed wild type non-human organism.

Accordingly, in one embodiment, an increase of 2-hydroxypalmitic acid in a non-human organism, as compared to a corresponding non-transformed wild type non-human organism, is conferred in the process of the invention, if the activity of a polypeptide showing the activity of a polygalacturonase, or if the activity of the polypeptide Yjr153w, preferably represented by SEQ ID NO. 66275, or a homolog or fragment thereof, or if the activity of a polypeptide encoded by a nucleic acid molecule comprising the nucleic acid SEQ ID NO. 66274, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 66274 or polypeptide SEQ ID NO. 66275, respectively, or a homolog or a fragment thereof, is increased or generated, or if the activity polygalacturonase is increased or generated in a non-human organism, like a microorganism or a plant cell, plant or part thereof, especially with non-targeted localization, whereby the respective line discloses in column 7 the fine chemical 2-hydroxypalmitic acid. For example, an increase of the 2-hydroxypalmitic acid of at least 1 percent, particularly in a range of 18 to 22-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a cyclin, or if the activity of the polypeptide Ylr210w, preferably represented by SEQ ID NO. 129066, or a homolog or fragment thereof, or if the activity of a polypeptide encoded by a nucleic acid molecule comprising the nucleic acid SEQ ID NO. 129065, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 129065 or polypeptide SEQ ID NO. 129066, respectively, or a homolog or a fragment thereof, is increased or generated, or if the activity cyclin is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 13 to 23-percent is conferred as compared to a corresponding non-transformed wild type non-human organism.

Accordingly, in one embodiment, an increase of hexadecadienoic acid in a non-human organism, as compared to a corresponding non-transformed wild type non-human organism, is conferred in the process of the invention, if the activity of a polypeptide showing the activity of a ylr326w-protein, or if the activity of the polypeptide Ylr326w, preferably represented by SEQ ID NO. 129124, or a homolog or fragment thereof, or if the activity of a polypeptide encoded by a nucleic acid molecule comprising the nucleic acid SEQ ID NO. 129123, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 129123 or polypeptide SEQ ID NO. 129124, respectively, or a homolog or a fragment thereof, is increased or generated, or if the activity ylr326w-protein is increased or generated in a non-human organism, like a microorganism or a plant cell, plant or part thereof, especially with plastidic localization, whereby the respective line discloses in column 7 the fine chemical hexadecadienoic acid. For example, an increase of the hexadecadienoic acid of at least 1 percent, particularly in a range of 21 to 94-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a ylr326w-protein, or if the activity of the polypeptide Ylr326w, preferably represented by SEQ ID NO. 129124, or a homolog or fragment thereof, or if the activity of a polypeptide encoded by a nucleic acid molecule comprising the nucleic acid SEQ ID NO. 129123, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 129123 or polypeptide SEQ ID NO. 129124, respectively, or a homolog or a fragment thereof, is increased or generated, or if the activity ylr326w-protein is increased or generated in a non-human organism, like a microorganism or a plant cell, plant or part thereof, especially with plastidic localization, whereby the respective line discloses in column 7 the fine chemical hexadecatrienoic acid. For example, an increase of the hexadecatrienoic acid of at least 1 percent, particularly in a range of 13 to 35-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a yml083c-protein, or if the activity of the polypeptide Yml083c, preferably represented by SEQ ID NO. 91782, or a homolog or fragment thereof, or if the activity of a polypeptide encoded by a nucleic acid molecule comprising the nucleic acid SEQ ID NO. 91781, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 91781 or polypeptide SEQ ID NO. 91782, respectively, or a homolog or a fragment thereof, is increased or generated, or if the activity yml083c-protein is increased or generated in a non-human organism, like a microorganism or a plant cell, plant or part thereof, especially with plastidic localization, whereby the respective line discloses in column 7 the fine chemical 2-hydroxybehenic acid. For example, an increase of the 2-hydroxybehenic acid of at least 1 percent, particularly in a range of 28 to 56-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a yml084w-protein, or if the activity of the polypeptide Yml084w, preferably represented by SEQ ID NO. 15176, or a homolog or fragment thereof, or if the activity of a polypeptide encoded by a nucleic acid molecule comprising the nucleic acid SEQ ID NO. 15175, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 15175 or polypeptide SEQ ID NO. 15176, respectively, or a homolog or a fragment thereof, is increased or generated, or if the activity yml084w-protein is increased or generated in a non-human organism, like a microorganism or a plant cell, plant or part thereof, especially with plastidic localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 26 to 59-percent is conferred as compared to a corresponding non-transformed wild type non-human organism.

Accordingly, in one embodiment, an increase of 2-hydroxypalmitic acid in a non-human organism, as compared to a corresponding non-transformed wild type non-human organism, is conferred in the process of the invention, if the activity of a polypeptide showing the activity of a molecular chaperone, or if the activity of the polypeptide Ymr186w, preferably represented by SEQ ID NO. 129142, or a homolog or fragment thereof, or if the activity of a polypeptide encoded by a nucleic acid molecule comprising the nucleic acid SEQ ID NO. 129141, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 129141 or polypeptide SEQ ID NO. 129142, respectively, or a homolog or a fragment thereof, is increased or generated, or if the activity molecular chaperone is increased or generated in a non-human organism, like a microorganism or a plant cell, plant or part thereof, especially with plastidic localization, whereby the respective line discloses in column 7 the fine chemical 2-hydroxypalmitic acid. For example, an increase of the 2-hydroxypalmitic acid of at least 1 percent, particularly in a range of 18 to 27-percent is conferred as compared to a corresponding non-transformed wild type non-human organism.

Accordingly, in one embodiment, an increase of oleic acid in a non-human organism, as compared to a corresponding non-transformed wild type non-human organism, is conferred in the process of the invention, if the activity of a polypeptide showing the activity of a phosphotransferase, or if the activity of the polypeptide Ynl130c, preferably represented by SEQ ID NO. 129471, or a homolog or fragment thereof, or if the activity of a polypeptide encoded by a nucleic acid molecule comprising the nucleic acid SEQ ID NO. 129470, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 129470 or polypeptide SEQ ID NO. 129471, respectively, or a homolog or a fragment thereof, is increased or generated, or if the activity phosphotransferase is increased or generated in a non-human organism, like a microorganism or a plant cell, plant or part thereof, especially with mitochondrial localization, whereby the respective line discloses in column 7 the fine chemical oleic acid. For example, an increase of the oleic acid of at least 1 percent, particularly in a range of 23 to 36-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a ynl321w-protein, or if the activity of the polypeptide Ynl321w, preferably represented by SEQ ID NO. 129547, or a homolog or fragment thereof, or if the activity of a polypeptide encoded by a nucleic acid molecule comprising the nucleic acid SEQ ID NO. 129546, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 129546 or polypeptide SEQ ID NO. 129547, respectively, or a homolog or a fragment thereof, is increased or generated, or if the activity ynl321w-protein is increased or generated in a non-human organism, like a microorganism or a plant cell, plant or part thereof, especially with plastidic localization, whereby the respective line discloses in column 7 the fine chemical 2-hydroxybehenic acid. For example, an increase of the 2-hydroxybehenic acid of at least 1 percent, particularly in a range of 26 to 53-percent is conferred as compared to a corresponding non-transformed wild type non-human organism.

Accordingly, in one embodiment, an increase of hexadecatrienoic acid in a non-human organism, as compared to a corresponding non-transformed wild type non-human organism, is conferred in the process of the invention, if the activity of a polypeptide showing the activity of a Elongation factor Tu, or if the activity of the polypeptide Yor187w, preferably represented by SEQ ID NO. 129575, or a homolog or fragment thereof, or if the activity of a polypeptide encoded by a nucleic acid molecule comprising the nucleic acid SEQ ID NO. 129574, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 129574 or polypeptide SEQ ID NO. 129575, respectively, or a homolog or a fragment thereof, is increased or generated, or if the activity Elongation factor Tu is increased or generated in a non-human organism, like a microorganism or a plant cell, plant or part thereof, especially with non-targeted localization, whereby the respective line discloses in column 7 the fine chemical hexadecatrienoic acid. For example, an increase of the hexadecatrienoic acid of at least 1 percent, particularly in a range of 17 to 27-percent is conferred as compared to a corresponding non-transformed wild type non-human organism.

Accordingly, in one embodiment, an increase of 2-hydroxytetracosenoic acid (preferably 2-hydroxynervonic acid) in a non-human organism, as compared to a corresponding non-transformed wild type non-human organism, is conferred in the process of the invention, if the activity of a polypeptide showing the activity of a ATPase epsilon subunit, or if the activity of the polypeptide Ypl271w, preferably represented by SEQ ID NO. 118213, or a homolog or fragment thereof, or if the activity of a polypeptide encoded by a nucleic acid molecule comprising the nucleic acid SEQ ID NO. 118212, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 118212 or polypeptide SEQ ID NO. 118213, respectively, or a homolog or a fragment thereof, is increased or generated, or if the activity ATPase epsilon subunit is increased or generated in a non-human organism, like a microorganism or a plant cell, plant or part thereof, especially with plastidic localization, whereby the respective line discloses in column 7 the fine chemical 2-hydroxytetracosenoic acid (preferably 2-hydroxynervonic acid). For example, an increase of the 2-hydroxytetracosenoic acid (preferably 2-hydroxynervonic acid) of at least 1 percent, particularly in a range of 25 to 75-percent is conferred as compared to a corresponding non-transformed wild type non-human organism.

Accordingly, in one embodiment, an increase of 2-hydroxypalmitic acid in a non-human organism, as compared to a corresponding non-transformed wild type non-human organism, is conferred in the process of the invention, if the activity of a polypeptide showing the activity of a phosphoadenosine phosphosulfate reductase, or if the activity of the polypeptide Ypr167c, preferably represented by SEQ ID NO. 130182, or a homolog or fragment thereof, or if the activity of a polypeptide encoded by a nucleic acid molecule comprising the nucleic acid SEQ ID NO. 130181, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 130181 or polypeptide SEQ ID NO. 130182, respectively, or a homolog or a fragment thereof, is increased or generated, or if the activity phosphoadenosine phosphosulfate reductase is increased or generated in a non-human organism, like a microorganism or a plant cell, plant or part thereof, especially with plastidic localization, whereby the respective line discloses in column 7 the fine chemical 2-hydroxypalmitic acid. For example, an increase of the 2-hydroxypalmitic acid of at least 1 percent, particularly in a range of 66 to 84-percent is conferred as compared to a corresponding non-transformed wild type non-human organism.

Accordingly, in one embodiment, an increase of oleic acid in a non-human organism, as compared to a corresponding non-transformed wild type non-human organism, is conferred in the process of the invention, if the activity of a polypeptide showing the activity of a 40S ribosomal protein S17, or if the activity of the polypeptide YDR447C, preferably represented by SEQ ID NO. 128162, or a homolog or fragment thereof, or if the activity of a polypeptide encoded by a nucleic acid molecule comprising the nucleic acid SEQ ID NO. 128161, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 128161 or polypeptide SEQ ID NO. 128162, respectively, or a homolog or a fragment thereof, is increased or generated, or if the activity 40S ribosomal protein S17 is increased or generated in a non-human organism, like a microorganism or a plant cell, plant or part thereof, especially with non-targeted localization, whereby the respective line discloses in column 7 the fine chemical oleic acid. For example, an increase of the oleic acid of at least 1 percent, particularly in a range of 25 to 395-percent is conferred as compared to a corresponding non-transformed wild type non-human organism.

Accordingly, in one embodiment, an increase of 2-hydroxybehenic acid in a non-human organism, as compared to a corresponding non-transformed wild type non-human organism, is conferred in the process of the invention, if the activity of a polypeptide showing the activity of a b3644-protein, or if the activity of the polypeptide B3644, preferably represented by SEQ ID NO. 125349, or a homolog or fragment thereof, or if the activity of a polypeptide encoded by a nucleic acid molecule comprising the nucleic acid SEQ ID NO. 125348, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 125348 or polypeptide SEQ ID NO. 125349, respectively, or a homolog or a fragment thereof, is increased or generated, or if the activity b3644-protein is increased or generated in a non-human organism, like a microorganism or a plant cell, plant or part thereof, especially with non-targeted localization, whereby the respective line discloses in column 7 the fine chemical 2-hydroxybehenic acid. For example, an increase of the 2-hydroxybehenic acid of at least 1 percent, particularly in a range of 2 to 77-percent is conferred as compared to a corresponding non-transformed wild type non-human organism.

Accordingly, in one embodiment, an increase of behenic acid in a non-human organism, as compared to a corresponding non-transformed wild type non-human organism, is conferred in the process of the invention, if the activity of a polypeptide showing the activity of a b3644-protein, or if the activity of the polypeptide B3644, preferably represented by SEQ ID NO. 125349, or a homolog or fragment thereof, or if the activity of a polypeptide encoded by a nucleic acid molecule comprising the nucleic acid SEQ ID NO. 125348, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 125348 or polypeptide SEQ ID NO. 125349, respectively, or a homolog or a fragment thereof, is increased or generated, or if the activity b3644-protein is increased or generated in a non-human organism, like a microorganism or a plant cell, plant or part thereof, especially with non-targeted localization, whereby the respective line discloses in column 7 the fine chemical behenic acid. For example, an increase of the behenic acid of at least 1 percent, particularly in a range of 0 to 58-percent is conferred as compared to a corresponding non-transformed wild type non-human organism.

Accordingly, in one embodiment, an increase of nervonic acid in a non-human organism, as compared to a corresponding non-transformed wild type non-human organism, is conferred in the process of the invention, if the activity of a polypeptide showing the activity of a b3644-protein, or if the activity of the polypeptide B3644, preferably represented by SEQ ID NO. 125349, or a homolog or fragment thereof, or if the activity of a polypeptide encoded by a nucleic acid molecule comprising the nucleic acid SEQ ID NO. 125348, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 12, column 5 or 8 in the respective same line as the nucleic acid molecule SEQ ID NO. 125348 or polypeptide SEQ ID NO. 125349, respectively, or a homolog or a fragment thereof, is increased or generated, or if the activity b3644-protein is increased or generated in a non-human organism, like a microorganism or a plant cell, plant or part thereof, especially with non-targeted localization, whereby the respective line discloses in column 7 the fine chemical nervonic acid. For example, an increase of the nervonic acid of at least 1 percent, particularly in a range of 12 to 319 percent is conferred as compared to a corresponding non-transformed wild type non-human organism.

[0096.1.8.12] to [0103.1.8.12] for the disclosure of these paragraphs see [0096.1.8.8] to [0103.1.8.8] above.

In this paragraph “A” means “Goffeau et al., Science 274 (5287), 546 (1996)”, “B” means “Blattner et al., Science 277 (5331), 1453 (1997)”, “C” means “Nakamura et al., Nucleic Acids Res. 28(1), 72 (2000)”, “D” means “deposition of the sequences to the EMBL/GenBank/DDBJ databases under “Sequencing of the draft genome assembly of Azotobacter vinelandii AvOP”. in June 2005 by Copeland et al.”, “E” means “Henne et al., Nat. Biotechnol. 22 (5), 547 (2004)”, “F” means “Kaul et al., Nature 408 (6814), 796 (2000)”, and “G” means “Matsumoto et al., Nature 436 (7052), 793 (2005)”.

The nucleic acid sequence of At1g19670 from Arabidopsis thaliana, e.g. as shown in the respective line in column 5 of Table I, application no. 12, has been published in F. And the activity of the gene product thereof is the activity of chlorophyllase.

Accordingly, in one embodiment, the process of the present invention for producing cerebronic acid in a non-human organism, like a microorganism or a plant or a part thereof, comprises increasing or generating the activity of a gene product with the activity of a gene product conferring the activity of “chlorophyllase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical cerebronic acid), application no. 12, preferably the coding region thereof, or a homolog or a fragment thereof, and being depicted in the same respective line as said At1g19670, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At1g19670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At1g19670, or a functional equivalent or a homolog thereof as depicted in column 8 of Table II, application no. 12, preferably a homolog or functional equivalent as depicted in column 8 of Table II B, application no. 12, and being depicted in the same respective line as said At1g19670, and preferably the activity is increased non-targeted, whereby the respective line discloses in column 7 the fine chemical cerebronic acid.

Accordingly, in one embodiment, the molecule which activity is to be increased in the process of the invention is the gene product with an activity as a “chlorophyllase”, preferably it is the molecule of section (a) or (b) of this paragraph.

In particular, it was observed that in plants, especially in Arabidopsis thaliana, increasing or generating the activity of a gene product non-targeted with the activity of a “chlorophyllase”, preferably being encoded by a gene comprising the nucleic acid sequence SEQ ID NO. 113651, preferably the coding region thereof, conferred the production of or the increase in cerebronic acid compared with the wild type control.

The nucleic acid sequence of At1g19670 from Arabidopsis thaliana, e.g. as shown in the respective line in column 5 of Table I, application no. 12, has been published in F. And the activity of the gene product thereof is the activity of chlorophyllase.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “chlorophyllase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said At1g19670, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At1g19670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At1g19670, or a functional equivalent or a homolog thereof as depicted in column 8 of Table II, application no. 12, preferably a homolog or functional equivalent as depicted in column 8 of Table II B, application no. 12, and being depicted in the same respective line as said At1g19670, and preferably the activity is increased non-targeted, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “chlorophyllase”, preferably it is the molecule of section (a) or (b) of this paragraph.

In particular, it was observed that in plants, especially in Arabidopsis thaliana, increasing or generating the activity of a gene product non-targeted with the activity of a “chlorophyllase”, preferably being encoded by a gene comprising the nucleic acid sequence SEQ ID NO. 113651, preferably the coding region thereof, conferred the production of or the increase in 2-hydroxytetracosenoic acid (preferably 2-hydroxynervonic acid) compared with the wild type control.

The nucleic acid sequence of At1g68320 from Arabidopsis thaliana, e.g. as shown in the respective line in column 5 of Table I, application no. 12, has been published in F. And the activity of the gene product thereof is the activity of transcription factor.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said At1g68320, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At1g68320, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At1g68320, or a functional equivalent or a homolog thereof as depicted in column 8 of Table II, application no. 12, preferably a homolog or functional equivalent as depicted in column 8 of Table II B, application no. 12, and being depicted in the same respective line as said At1g68320, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 1061, preferably the coding region thereof, conferred the production of or the increase in hexadecatrienoic acid compared with the wild type control.

The nucleic acid sequence of At1g72770 from Arabidopsis thaliana, e.g. as shown in the respective line in column 5 of Table I, application no. 12, has been published in F. And the activity of the gene product thereof is the activity of protein phosphatase.

Accordingly, in one embodiment, the process of the present invention for producing cerebronic acid in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical cerebronic acid), application no. 12, preferably the coding region thereof, or a homolog or a fragment thereof, and being depicted in the same respective line as said At1g72770, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At1g72770, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At1g72770, or a functional equivalent or a homolog thereof as depicted in column 8 of Table II, application no. 12, preferably a homolog or functional equivalent as depicted in column 8 of Table II B, application no. 12, and being depicted in the same respective line as said At1g72770, and preferably the activity is increased non-targeted, whereby the respective line discloses in column 7 the fine chemical cerebronic acid.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19419, preferably the coding region thereof, conferred the production of or the increase in cerebronic acid compared with the wild type control.

The nucleic acid sequence of At1g72770 from Arabidopsis thaliana, e.g. as shown in the respective line in column 5 of Table I, application no. 12, has been published in F. And the activity of the gene product thereof is the activity of protein phosphatase.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said At1g72770, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At1g72770, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At1g72770, or a functional equivalent or a homolog thereof as depicted in column 8 of Table II, application no. 12, preferably a homolog or functional equivalent as depicted in column 8 of Table II B, application no. 12, and being depicted in the same respective line as said At1g72770, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19419, preferably the coding region thereof, conferred the production of or the increase in hexadecatrienoic acid compared with the wild type control.

The nucleic acid sequence of At1g72770 from Arabidopsis thaliana, e.g. as shown in the respective line in column 5 of Table I, application no. 12, has been published in F. And the activity of the gene product thereof is the activity of protein phosphatase.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said At1g72770, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At1g72770, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At1g72770, or a functional equivalent or a homolog thereof as depicted in column 8 of Table II, application no. 12, preferably a homolog or functional equivalent as depicted in column 8 of Table II B, application no. 12, and being depicted in the same respective line as said At1g72770, and preferably the activity is increased non-targeted, whereby the respective line discloses in column 7 the fine chemical 2-hydroxybehenic acid.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19419, preferably the coding region thereof, conferred the production of or the increase in 2-hydroxybehenic acid compared with the wild type control.

The nucleic acid sequence of At2g33510 from Arabidopsis thaliana, e.g. as shown in the respective line in column 5 of Table I, application no. 12, has been published in F. And the activity of the gene product thereof is the activity of At2g33510-protein.

Accordingly, in one embodiment, the process of the present invention for producing 2-hydroxypalmitic acid in a non-human organism, like a microorganism or a plant or a part thereof, comprises increasing or generating the activity of a gene product with the activity of a gene product conferring the activity of “At2g33510-protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxypalmitic acid), application no. 12, preferably the coding region thereof, or a homolog or a fragment thereof, and being depicted in the same respective line as said At2g33510, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At2g33510, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At2g33510, or a functional equivalent or a homolog thereof as depicted in column 8 of Table II, application no. 12, preferably a homolog or functional equivalent as depicted in column 8 of Table II B, application no. 12, and being depicted in the same respective line as said At2g33510, and preferably the activity is increased non-targeted, whereby the respective line discloses in column 7 the fine chemical 2-hydroxypalmitic acid.

Accordingly, in one embodiment, the molecule which activity is to be increased in the process of the invention is the gene product with an activity as a “At2g33510-protein”, preferably it is the molecule of section (a) or (b) of this paragraph.

In particular, it was observed that in plants, especially in Arabidopsis thaliana, increasing or generating the activity of a gene product non-targeted with the activity of a “At2g33510-protein”, preferably being encoded by a gene comprising the nucleic acid sequence SEQ ID NO. 98522, preferably the coding region thereof, conferred the production of or the increase in 2-hydroxypalmitic acid compared with the wild type control.

The nucleic acid sequence of At3g02990 from Arabidopsis thaliana, e.g. as shown in the respective line in column 5 of Table I, application no. 12, has been published in F. And the activity of the gene product thereof is the activity of heat shock transcription factor.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said At3g02990, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At3g02990, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At3g02990, or a functional equivalent or a homolog thereof as depicted in column 8 of Table II, application no. 12, preferably a homolog or functional equivalent as depicted in column 8 of Table II B, application no. 12, and being depicted in the same respective line as said At3g02990, and preferably the activity is increased non-targeted, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 68657, preferably the coding region thereof, conferred the production of or the increase in 2-hydroxytetracosenoic acid (preferably 2-hydroxynervonic acid) compared with the wild type control.

The nucleic acid sequence of At3g09820 from Arabidopsis thaliana, e.g. as shown in the respective line in column 5 of Table I, application no. 12, has been published in F. And the activity of the gene product thereof is the activity of adenosine kinase.

Accordingly, in one embodiment, the process of the present invention for producing cerebronic acid in a non-human organism, like a microorganism or a plant or a part thereof, comprises increasing or generating the activity of a gene product with the activity of a gene product conferring the activity of “adenosine kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical cerebronic acid), application no. 12, preferably the coding region thereof, or a homolog or a fragment thereof, and being depicted in the same respective line as said At3g09820, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At3g09820, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At3g09820, or a functional equivalent or a homolog thereof as depicted in column 8 of Table II, application no. 12, preferably a homolog or functional equivalent as depicted in column 8 of Table II B, application no. 12, and being depicted in the same respective line as said At3g09820, and preferably the activity is increased non-targeted, whereby the respective line discloses in column 7 the fine chemical cerebronic acid.

Accordingly, in one embodiment, the molecule which activity is to be increased in the process of the invention is the gene product with an activity as a “adenosine kinase”, preferably it is the molecule of section (a) or (b) of this paragraph.

In particular, it was observed that in plants, especially in Arabidopsis thaliana, increasing or generating the activity of a gene product non-targeted with the activity of a “adenosine kinase”, preferably being encoded by a gene comprising the nucleic acid sequence SEQ ID NO. 1696, preferably the coding region thereof, conferred the production of or the increase in cerebronic acid compared with the wild type control.

The nucleic acid sequence of At3g09820 from Arabidopsis thaliana, e.g. as shown in the respective line in column 5 of Table I, application no. 12, has been published in F. And the activity of the gene product thereof is the activity of adenosine kinase.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “adenosine kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said At3g09820, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At3g09820, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At3g09820, or a functional equivalent or a homolog thereof as depicted in column 8 of Table II, application no. 12, preferably a homolog or functional equivalent as depicted in column 8 of Table II B, application no. 12, and being depicted in the same respective line as said At3g09820, and preferably the activity is increased non-targeted, whereby the respective line discloses in column 7 the fine chemical 2-hydroxybehenic acid.

Accordingly, in one embodiment, the molecule which activity is to be increased in the process of the invention is the gene product with an activity as a “adenosine kinase”, preferably it is the molecule of section (a) or (b) of this paragraph.

In particular, it was observed that in plants, especially in Arabidopsis thaliana, increasing or generating the activity of a gene product non-targeted with the activity of a “adenosine kinase”, preferably being encoded by a gene comprising the nucleic acid sequence SEQ ID NO. 1696, preferably the coding region thereof, conferred the production of or the increase in 2-hydroxybehenic acid compared with the wild type control.

The nucleic acid sequence of At3g18524 from Arabidopsis thaliana, e.g. as shown in the respective line in column 5 of Table I, application no. 12, has been published in F. And the activity of the gene product thereof is the activity of DNA mismatch repair protein.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “DNA mismatch repair protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said At3g18524, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At3g18524, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At3g18524, or a functional equivalent or a homolog thereof as depicted in column 8 of Table II, application no. 12, preferably a homolog or functional equivalent as depicted in column 8 of Table II B, application no. 12, and being depicted in the same respective line as said At3g18524, and preferably the activity is increased non-targeted, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “DNA mismatch repair protein”, preferably it is the molecule of section (a) or (b) of this paragraph.

In particular, it was observed that in plants, especially in Arabidopsis thaliana, increasing or generating the activity of a gene product non-targeted with the activity of a “DNA mismatch repair protein”, preferably being encoded by a gene comprising the nucleic acid sequence SEQ ID NO. 22832, preferably the coding region thereof, conferred the production of or the increase in 2-hydroxytetracosenoic acid (preferably 2-hydroxynervonic acid) compared with the wild type control.

The nucleic acid sequence of At3g62950 from Arabidopsis thaliana, e.g. as shown in the respective line in column 5 of Table I, application no. 12, has been published in F. And the activity of the gene product thereof is the activity of glutaredoxin.

Accordingly, in one embodiment, the process of the present invention for producing cerebronic acid in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical cerebronic acid), application no. 12, preferably the coding region thereof, or a homolog or a fragment thereof, and being depicted in the same respective line as said At3g62950, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At3g62950, or a functional equivalent or a homolog thereof as depicted in column 8 of Table II, application no. 12, preferably a homolog or functional equivalent as depicted in column 8 of Table II B, application no. 12, and being depicted in the same respective line as said At3g62950, and preferably the activity is increased non-targeted, whereby the respective line discloses in column 7 the fine chemical cerebronic acid.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in cerebronic acid compared with the wild type control.

The nucleic acid sequence of At3g62950 from Arabidopsis thaliana, e.g. as shown in the respective line in column 5 of Table I, application no. 12, has been published in F. And the activity of the gene product thereof is the activity of glutaredoxin.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said At3g62950, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At3g62950, or a functional equivalent or a homolog thereof as depicted in column 8 of Table II, application no. 12, preferably a homolog or functional equivalent as depicted in column 8 of Table II B, application no. 12, and being depicted in the same respective line as said At3g62950, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in hexadecatrienoic acid compared with the wild type control.

The nucleic acid sequence of At3g62950 from Arabidopsis thaliana, e.g. as shown in the respective line in column 5 of Table I, application no. 12, has been published in F. And the activity of the gene product thereof is the activity of glutaredoxin.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said At3g62950, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At3g62950, or a functional equivalent or a homolog thereof as depicted in column 8 of Table II, application no. 12, preferably a homolog or functional equivalent as depicted in column 8 of Table II B, application no. 12, and being depicted in the same respective line as said At3g62950, and preferably the activity is increased non-targeted, whereby the respective line discloses in column 7 the fine chemical 2-hydroxybehenic acid.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in 2-hydroxybehenic acid compared with the wild type control.

The nucleic acid sequence of At4g09960 from Arabidopsis thaliana, e.g. as shown in the respective line in column 5 of Table I, application no. 12, has been published in F. And the activity of the gene product thereof is the activity of transcription factor.

Accordingly, in one embodiment, the process of the present invention for producing 2-hydroxypalmitic acid in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxypalmitic acid), application no. 12, preferably the coding region thereof, or a homolog or a fragment thereof, and being depicted in the same respective line as said At4g09960, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At4g09960, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At4g09960, or a functional equivalent or a homolog thereof as depicted in column 8 of Table II, application no. 12, preferably a homolog or functional equivalent as depicted in column 8 of Table II B, application no. 12, and being depicted in the same respective line as said At4g09960, and preferably the activity is increased non-targeted, whereby the respective line discloses in column 7 the fine chemical 2-hydroxypalmitic acid.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 109717, preferably the coding region thereof, conferred the production of or the increase in 2-hydroxypalmitic acid compared with the wild type control.

The nucleic acid sequence of At4g15660 from Arabidopsis thaliana, e.g. as shown in the respective line in column 5 of Table I, application no. 12, has been published in F. And the activity of the gene product thereof is the activity of monothiol glutaredoxin.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said At4g15660, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At4g15660, or a functional equivalent or a homolog thereof as depicted in column 8 of Table II, application no. 12, preferably a homolog or functional equivalent as depicted in column 8 of Table II B, application no. 12, and being depicted in the same respective line as said At4g15660, and preferably the activity is increased non-targeted, whereby the respective line discloses in column 7 the fine chemical 2-hydroxybehenic acid.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in 2-hydroxybehenic acid compared with the wild type control.

The nucleic acid sequence of At4g15660 from Arabidopsis thaliana, e.g. as shown in the respective line in column 5 of Table I, application no. 12, has been published in F. And the activity of the gene product thereof is the activity of monothiol glutaredoxin.

Accordingly, in one embodiment, the process of the present invention for producing 2-hydroxypalmitic acid in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxypalmitic acid), application no. 12, preferably the coding region thereof, or a homolog or a fragment thereof, and being depicted in the same respective line as said At4g15660, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At4g15660, or a functional equivalent or a homolog thereof as depicted in column 8 of Table II, application no. 12, preferably a homolog or functional equivalent as depicted in column 8 of Table II B, application no. 12, and being depicted in the same respective line as said At4g15660, and preferably the activity is increased non-targeted, whereby the respective line discloses in column 7 the fine chemical 2-hydroxypalmitic acid.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in 2-hydroxypalmitic acid compared with the wild type control.

The nucleic acid sequence of At4g15670 from Arabidopsis thaliana, e.g. as shown in the respective line in column 5 of Table I, application no. 12, has been published in F. And the activity of the gene product thereof is the activity of monothiol glutaredoxin.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said At4g15670, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At4g15670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At4g15670, or a functional equivalent or a homolog thereof as depicted in column 8 of Table II, application no. 12, preferably a homolog or functional equivalent as depicted in column 8 of Table II B, application no. 12, and being depicted in the same respective line as said At4g15670, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3279, preferably the coding region thereof, conferred the production of or the increase in hexadecatrienoic acid compared with the wild type control.

The nucleic acid sequence of At4g15670 from Arabidopsis thaliana, e.g. as shown in the respective line in column 5 of Table I, application no. 12, has been published in F. And the activity of the gene product thereof is the activity of monothiol glutaredoxin.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said At4g15670, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At4g15670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At4g15670, or a functional equivalent or a homolog thereof as depicted in column 8 of Table II, application no. 12, preferably a homolog or functional equivalent as depicted in column 8 of Table II B, application no. 12, and being depicted in the same respective line as said At4g15670, and preferably the activity is increased non-targeted, whereby the respective line discloses in column 7 the fine chemical 2-hydroxybehenic acid.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3279, preferably the coding region thereof, conferred the production of or the increase in 2-hydroxybehenic acid compared with the wild type control.

The nucleic acid sequence of At4g15690 from Arabidopsis thaliana, e.g. as shown in the respective line in column 5 of Table I, application no. 12, has been published in F. And the activity of the gene product thereof is the activity of monothiol glutaredoxin.

Accordingly, in one embodiment, the process of the present invention for producing cerebronic acid in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical cerebronic acid), application no. 12, preferably the coding region thereof, or a homolog or a fragment thereof, and being depicted in the same respective line as said At4g15690, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At4g15690, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At4g15690, or a functional equivalent or a homolog thereof as depicted in column 8 of Table II, application no. 12, preferably a homolog or functional equivalent as depicted in column 8 of Table II B, application no. 12, and being depicted in the same respective line as said At4g15690, and preferably the activity is increased non-targeted, whereby the respective line discloses in column 7 the fine chemical cerebronic acid.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23844, preferably the coding region thereof, conferred the production of or the increase in cerebronic acid compared with the wild type control.

The nucleic acid sequence of At4g26080 from Arabidopsis thaliana, e.g. as shown in the respective line in column 5 of Table I, application no. 12, has been published in F. And the activity of the gene product thereof is the activity of protein phosphatase.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said At4g26080, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At4g26080, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At4g26080, or a functional equivalent or a homolog thereof as depicted in column 8 of Table II, application no. 12, preferably a homolog or functional equivalent as depicted in column 8 of Table II B, application no. 12, and being depicted in the same respective line as said At4g26080, and preferably the activity is increased non-targeted, whereby the respective line discloses in column 7 the fine chemical 2-hydroxybehenic acid.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 68849, preferably the coding region thereof, conferred the production of or the increase in 2-hydroxybehenic acid compared with the wild type control.

The nucleic acid sequence of At4g26080 from Arabidopsis thaliana, e.g. as shown in the respective line in column 5 of Table I, application no. 12, has been published in F. And the activity of the gene product thereof is the activity of protein phosphatase.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said At4g26080, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At4g26080, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At4g26080, or a functional equivalent or a homolog thereof as depicted in column 8 of Table II, application no. 12, preferably a homolog or functional equivalent as depicted in column 8 of Table II B, application no. 12, and being depicted in the same respective line as said At4g26080, and preferably the activity is increased non-targeted, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 68849, preferably the coding region thereof, conferred the production of or the increase in 2-hydroxytetracosenoic acid (preferably 2-hydroxynervonic acid) compared with the wild type control.

The nucleic acid sequence of At4g33040 from Arabidopsis thaliana, e.g. as shown in the respective line in column 5 of Table I, application no. 12, has been published in F. And the activity of the gene product thereof is the activity of glutaredoxin.

Accordingly, in one embodiment, the process of the present invention for producing cerebronic acid in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical cerebronic acid), application no. 12, preferably the coding region thereof, or a homolog or a fragment thereof, and being depicted in the same respective line as said At4g33040, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At4g33040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At4g33040, or a functional equivalent or a homolog thereof as depicted in column 8 of Table II, application no. 12, preferably a homolog or functional equivalent as depicted in column 8 of Table II B, application no. 12, and being depicted in the same respective line as said At4g33040, and preferably the activity is increased non-targeted, whereby the respective line discloses in column 7 the fine chemical cerebronic acid.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 4102, preferably the coding region thereof, conferred the production of or the increase in cerebronic acid compared with the wild type control.

The nucleic acid sequence of At4g33040 from Arabidopsis thaliana, e.g. as shown in the respective line in column 5 of Table I, application no. 12, has been published in F. And the activity of the gene product thereof is the activity of glutaredoxin.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said At4g33040, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At4g33040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At4g33040, or a functional equivalent or a homolog thereof as depicted in column 8 of Table II, application no. 12, preferably a homolog or functional equivalent as depicted in column 8 of Table II B, application no. 12, and being depicted in the same respective line as said At4g33040, and preferably the activity is increased non-targeted, whereby the respective line discloses in column 7 the fine chemical hexadecadienoic acid.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 4102, preferably the coding region thereof, conferred the production of or the increase in hexadecadienoic acid compared with the wild type control.

The nucleic acid sequence of At4g33040 from Arabidopsis thaliana, e.g. as shown in the respective line in column 5 of Table I, application no. 12, has been published in F. And the activity of the gene product thereof is the activity of glutaredoxin.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said At4g33040, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At4g33040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At4g33040, or a functional equivalent or a homolog thereof as depicted in column 8 of Table II, application no. 12, preferably a homolog or functional equivalent as depicted in column 8 of Table II B, application no. 12, and being depicted in the same respective line as said At4g33040, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 4102, preferably the coding region thereof, conferred the production of or the increase in hexadecatrienoic acid compared with the wild type control.

The nucleic acid sequence of At4g35310 from Arabidopsis thaliana, e.g. as shown in the respective line in column 5 of Table I, application no. 12, has been published in F. And the activity of the gene product thereof is the activity of calcium-dependent protein kinase.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said At4g35310, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At4g35310, or a functional equivalent or a homolog thereof as depicted in column 8 of Table II, application no. 12, preferably a homolog or functional equivalent as depicted in column 8 of Table II B, application no. 12, and being depicted in the same respective line as said At4g35310, and preferably the activity is increased non-targeted, whereby the respective line discloses in column 7 the fine chemical 2-hydroxybehenic acid.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in 2-hydroxybehenic acid compared with the wild type control.

The nucleic acid sequence of At4g35310 from Arabidopsis thaliana, e.g. as shown in the respective line in column 5 of Table I, application no. 12, has been published in F. And the activity of the gene product thereof is the activity of calcium-dependent protein kinase.

Accordingly, in one embodiment, the process of the present invention for producing 2-hydroxypalmitic acid in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxypalmitic acid), application no. 12, preferably the coding region thereof, or a homolog or a fragment thereof, and being depicted in the same respective line as said At4g35310, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At4g35310, or a functional equivalent or a homolog thereof as depicted in column 8 of Table II, application no. 12, preferably a homolog or functional equivalent as depicted in column 8 of Table II B, application no. 12, and being depicted in the same respective line as said At4g35310, and preferably the activity is increased non-targeted, whereby the respective line discloses in column 7 the fine chemical 2-hydroxypalmitic acid.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in 2-hydroxypalmitic acid compared with the wild type control.

The nucleic acid sequence of At5g16230 from Arabidopsis thaliana, e.g. as shown in the respective line in column 5 of Table I, application no. 12, has been published in F. And the activity of the gene product thereof is the activity of acyl-desaturase.

Accordingly, in one embodiment, the process of the present invention for producing oleic acid in a non-human organism, like a microorganism or a plant or a part thereof, comprises increasing or generating the activity of a gene product with the activity of a gene product conferring the activity of “acyl-desaturase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical oleic acid), application no. 12, preferably the coding region thereof, or a homolog or a fragment thereof, and being depicted in the same respective line as said At5g16230, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At5g16230, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At5g16230, or a functional equivalent or a homolog thereof as depicted in column 8 of Table II, application no. 12, preferably a homolog or functional equivalent as depicted in column 8 of Table II B, application no. 12, and being depicted in the same respective line as said At5g16230, and preferably the activity is increased non-targeted, whereby the respective line discloses in column 7 the fine chemical oleic acid.

Accordingly, in one embodiment, the molecule which activity is to be increased in the process of the invention is the gene product with an activity as a “acyl-desaturase”, preferably it is the molecule of section (a) or (b) of this paragraph.

In particular, it was observed that in plants, especially in Arabidopsis thaliana, increasing or generating the activity of a gene product non-targeted with the activity of a “acyl-desaturase”, preferably being encoded by a gene comprising the nucleic acid sequence SEQ ID NO. 122565, preferably the coding region thereof, conferred the production of or the increase in oleic acid compared with the wild type control.

The nucleic acid sequence of At5g18600 from Arabidopsis thaliana, e.g. as shown in the respective line in column 5 of Table I, application no. 12, has been published in F. And the activity of the gene product thereof is the activity of monothiol glutaredoxin.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said At5g18600, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At5g18600, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At5g18600, or a functional equivalent or a homolog thereof as depicted in column 8 of Table II, application no. 12, preferably a homolog or functional equivalent as depicted in column 8 of Table II B, application no. 12, and being depicted in the same respective line as said At5g18600, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 4904, preferably the coding region thereof, conferred the production of or the increase in hexadecatrienoic acid compared with the wild type control.

The nucleic acid sequence of At5g57050 from Arabidopsis thaliana, e.g. as shown in the respective line in column 5 of Table I, application no. 12, has been published in F. And the activity of the gene product thereof is the activity of protein phosphatase.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said At5g57050, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At5g57050, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At5g57050, or a functional equivalent or a homolog thereof as depicted in column 8 of Table II, application no. 12, preferably a homolog or functional equivalent as depicted in column 8 of Table II B, application no. 12, and being depicted in the same respective line as said At5g57050, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 5318, preferably the coding region thereof, conferred the production of or the increase in hexadecatrienoic acid compared with the wild type control.

The nucleic acid sequence of At5g66710 from Arabidopsis thaliana, e.g. as shown in the respective line in column 5 of Table I, application no. 12, has been published in F. And the activity of the gene product thereof is the activity of protein kinase.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said At5g66710, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said At5g66710, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said At5g66710, or a functional equivalent or a homolog thereof as depicted in column 8 of Table II, application no. 12, preferably a homolog or functional equivalent as depicted in column 8 of Table II B, application no. 12, and being depicted in the same respective line as said At5g66710, and preferably the activity is increased non-targeted, whereby the respective line discloses in column 7 the fine chemical hexadecadienoic acid.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 122803, preferably the coding region thereof, conferred the production of or the increase in hexadecadienoic acid compared with the wild type control.

The nucleic acid sequence of AvinDRAFT_{—}0035 from Azotobacter vinelandii, e.g. as shown in the respective line in column 5 of Table I, application no. 12, has been published in D. And the activity of the gene product thereof is the activity of CDP-diacylglycerol-serine O-phosphatidyltransferase.

Accordingly, in one embodiment, the process of the present invention for producing cerebronic acid in a non-human organism, like a microorganism or a plant or a part thereof, comprises increasing or generating the activity of a gene product with the activity of a gene product conferring the activity of “CDP-diacylglycerol-serine O-phosphatidyltransferase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical cerebronic acid), application no. 12, preferably the coding region thereof, or a homolog or a fragment thereof, and being depicted in the same respective line as said AvinDRAFT_{—}0035, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said AvinDRAFT_{—}0035, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said AvinDRAFT_{—}0035, or a functional equivalent or a homolog thereof as depicted in column 8 of Table II, application no. 12, preferably a homolog or functional equivalent as depicted in column 8 of Table II B, application no. 12, and being depicted in the same respective line as said AvinDRAFT_{—}0035, and preferably the activity is increased non-targeted, whereby the respective line discloses in column 7 the fine chemical cerebronic acid.

Accordingly, in one embodiment, the molecule which activity is to be increased in the process of the invention is the gene product with an activity as a “CDP-diacylglycerol-serine O-phosphatidyltransferase”, preferably it is the molecule of section (a) or (b) of this paragraph.

In particular, it was observed that in plants, especially in Arabidopsis thaliana, increasing or generating the activity of a gene product non-targeted with the activity of a “CDP-diacylglycerol-serine O-phosphatidyltransferase”, preferably being encoded by a gene comprising the nucleic acid sequence SEQ ID NO. 70583, preferably the coding region thereof, conferred the production of or the increase in cerebronic acid compared with the wild type control.

The nucleic acid sequence of AvinDRAFT_{—}0035 from Azotobacter vinelandii, e.g. as shown in the respective line in column 5 of Table I, application no. 12, has been published in D. And the activity of the gene product thereof is the activity of CDP-diacylglycerol-serine O-phosphatidyltransferase.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “CDP-diacylglycerol-serine O-phosphatidyltransferase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said AvinDRAFT_{—}0035, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said AvinDRAFT_{—}0035, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said AvinDRAFT_{—}0035, or a functional equivalent or a homolog thereof as depicted in column 8 of Table II, application no. 12, preferably a homolog or functional equivalent as depicted in column 8 of Table II B, application no. 12, and being depicted in the same respective line as said AvinDRAFT_{—}0035, and preferably the activity is increased non-targeted, whereby the respective line discloses in column 7 the fine chemical 2-hydroxybehenic acid.

Accordingly, in one embodiment, the molecule which activity is to be increased in the process of the invention is the gene product with an activity as a “CDP-diacylglycerol-serine O-phosphatidyltransferase”, preferably it is the molecule of section (a) or (b) of this paragraph.

In particular, it was observed that in plants, especially in Arabidopsis thaliana, increasing or generating the activity of a gene product non-targeted with the activity of a “CDP-diacylglycerol-serine O-phosphatidyltransferase”, preferably being encoded by a gene comprising the nucleic acid sequence SEQ ID NO. 70583, preferably the coding region thereof, conferred the production of or the increase in 2-hydroxybehenic acid compared with the wild type control.

The nucleic acid sequence of AvinDRAFT_{—}0035 from Azotobacter vinelandii, e.g. as shown in the respective line in column 5 of Table I, application no. 12, has been published in D. And the activity of the gene product thereof is the activity of CDP-diacylglycerol-serine 0-phosphatidyltransferase.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “CDP-diacylglycerol-serine O-phosphatidyltransferase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said AvinDRAFT_{—}0035, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said AvinDRAFT_{—}0035, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said AvinDRAFT_{—}0035, or a functional equivalent or a homolog thereof as depicted in column 8 of Table II, application no. 12, preferably a homolog or functional equivalent as depicted in column 8 of Table II B, application no. 12, and being depicted in the same respective line as said AvinDRAFT_{—}0035, and preferably the activity is increased non-targeted, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “CDP-diacylglycerol-serine O-phosphatidyltransferase”, preferably it is the molecule of section (a) or (b) of this paragraph.

In particular, it was observed that in plants, especially in Arabidopsis thaliana, increasing or generating the activity of a gene product non-targeted with the activity of a “CDP-diacylglycerol-serine O-phosphatidyltransferase”, preferably being encoded by a gene comprising the nucleic acid sequence SEQ ID NO. 70583, preferably the coding region thereof, conferred the production of or the increase in 2-hydroxytetracosenoic acid (preferably 2-hydroxynervonic acid) compared with the wild type control.

The nucleic acid sequence of AvinDRAFT_{—}0035 from Azotobacter vinelandii, e.g. as shown in the respective line in column 5 of Table I, application no. 12, has been published in D. And the activity of the gene product thereof is the activity of CDP-diacylglycerol-serine O-phosphatidyltransferase.

Accordingly, in one embodiment, the process of the present invention for producing 2-hydroxypalmitic acid in a non-human organism, like a microorganism or a plant or a part thereof, comprises increasing or generating the activity of a gene product with the activity of a gene product conferring the activity of “CDP-diacylglycerol-serine O-phosphatidyltransferase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxypalmitic acid), application no. 12, preferably the coding region thereof, or a homolog or a fragment thereof, and being depicted in the same respective line as said AvinDRAFT_{—}0035, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said AvinDRAFT_{—}0035, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said AvinDRAFT_{—}0035, or a functional equivalent or a homolog thereof as depicted in column 8 of Table II, application no. 12, preferably a homolog or functional equivalent as depicted in column 8 of Table II B, application no. 12, and being depicted in the same respective line as said AvinDRAFT_{—}0035, and preferably the activity is increased non-targeted, whereby the respective line discloses in column 7 the fine chemical 2-hydroxypalmitic acid.

Accordingly, in one embodiment, the molecule which activity is to be increased in the process of the invention is the gene product with an activity as a “CDP-diacylglycerol-serine O-phosphatidyltransferase”, preferably it is the molecule of section (a) or (b) of this paragraph.

In particular, it was observed that in plants, especially in Arabidopsis thaliana, increasing or generating the activity of a gene product non-targeted with the activity of a “CDP-diacylglycerol-serine O-phosphatidyltransferase”, preferably being encoded by a gene comprising the nucleic acid sequence SEQ ID NO. 70583, preferably the coding region thereof, conferred the production of or the increase in 2-hydroxypalmitic acid compared with the wild type control.

The nucleic acid sequence of AvinDRAFT_{—}1624 from Azotobacter vinelandii, e.g. as shown in the respective line in column 5 of Table I, application no. 12, has been published in D. And the activity of the gene product thereof is the activity of sec-independent protein translocase.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “sec-independent protein translocase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said AvinDRAFT_{—}1624, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said AvinDRAFT_{—}1624, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said AvinDRAFT_{—}1624, or a functional equivalent or a homolog thereof as depicted in column 8 of Table II, application no. 12, preferably a homolog or functional equivalent as depicted in column 8 of Table II B, application no. 12, and being depicted in the same respective line as said AvinDRAFT_{—}1624, and preferably the activity is increased non-targeted, whereby the respective line discloses in column 7 the fine chemical hexadecadienoic acid.

Accordingly, in one embodiment, the molecule which activity is to be increased in the process of the invention is the gene product with an activity as a “sec-independent protein translocase”, preferably it is the molecule of section (a) or (b) of this paragraph.

In particular, it was observed that in plants, especially in Arabidopsis thaliana, increasing or generating the activity of a gene product non-targeted with the activity of a “sec-independent protein translocase”, preferably being encoded by a gene comprising the nucleic acid sequence SEQ ID NO. 26196, preferably the coding region thereof, conferred the production of or the increase in hexadecadienoic acid compared with the wild type control.

The nucleic acid sequence of AvinDRAFT_{—}2010 from Azotobacter vinelandii, e.g. as shown in the respective line in column 5 of Table I, application no. 12, has been published in D. And the activity of the gene product thereof is the activity of transcriptional regulator protein.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “transcriptional regulator protein”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said AvinDRAFT_{—}2010, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said AvinDRAFT_{—}2010, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said AvinDRAFT_{—}2010, or a functional equivalent or a homolog thereof as depicted in column 8 of Table II, application no. 12, preferably a homolog or functional equivalent as depicted in column 8 of Table II B, application no. 12, and being depicted in the same respective line as said AvinDRAFT_{—}2010, and preferably the activity is increased non-targeted, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “transcriptional regulator protein”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator protein”, preferably being encoded by a gene comprising the nucleic acid sequence SEQ ID NO. 114198, preferably the coding region thereof, conferred the production of or the increase in 2-hydroxytetracosenoic acid (preferably 2-hydroxynervonic acid) compared with the wild type control.

The nucleic acid sequence of AvinDRAFT_{—}2091 from Azotobacter vinelandii, e.g. as shown in the respective line in column 5 of Table I, application no. 12, has been published in D. And the activity of the gene product thereof is the activity of beta-hydroxylase.

Accordingly, in one embodiment, the process of the present invention for producing 2-hydroxypalmitic acid in a non-human organism, like a microorganism or a plant or a part thereof, comprises increasing or generating the activity of a gene product with the activity of a gene product conferring the activity of “beta-hydroxylase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxypalmitic acid), application no. 12, preferably the coding region thereof, or a homolog or a fragment thereof, and being depicted in the same respective line as said AvinDRAFT_{—}2091, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said AvinDRAFT_{—}2091, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said AvinDRAFT_{—}2091, or a functional equivalent or a homolog thereof as depicted in column 8 of Table II, application no. 12, preferably a homolog or functional equivalent as depicted in column 8 of Table II B, application no. 12, and being depicted in the same respective line as said AvinDRAFT_{—}2091, and preferably the activity is increased non-targeted, whereby the respective line discloses in column 7 the fine chemical 2-hydroxypalmitic acid.

Accordingly, in one embodiment, the molecule which activity is to be increased in the process of the invention is the gene product with an activity as a “beta-hydroxylase”, preferably it is the molecule of section (a) or (b) of this paragraph.

In particular, it was observed that in plants, especially in Arabidopsis thaliana, increasing or generating the activity of a gene product non-targeted with the activity of a “beta-hydroxylase”, preferably being encoded by a gene comprising the nucleic acid sequence SEQ ID NO. 6040, preferably the coding region thereof, conferred the production of or the increase in 2-hydroxypalmitic acid compared with the wild type control.

The nucleic acid sequence of AvinDRAFT_{—}2365 from Azotobacter vinelandii, e.g. as shown in the respective line in column 5 of Table I, application no. 12, has been published in D. And the activity of the gene product thereof is the activity of 30S ribosomal protein S3.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “30S ribosomal protein S3”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said AvinDRAFT_{—}2365, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said AvinDRAFT_{—}2365, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said AvinDRAFT_{—}2365, or a functional equivalent or a homolog thereof as depicted in column 8 of Table II, application no. 12, preferably a homolog or functional equivalent as depicted in column 8 of Table II B, application no. 12, and being depicted in the same respective line as said AvinDRAFT_{—}2365, and preferably the activity is increased non-targeted, whereby the respective line discloses in column 7 the fine chemical hexadecadienoic acid.

Accordingly, in one embodiment, the molecule which activity is to be increased in the process of the invention is the gene product with an activity as a “305 ribosomal protein S3”, preferably it is the molecule of section (a) or (b) of this paragraph.

In particular, it was observed that in plants, especially in Arabidopsis thaliana, increasing or generating the activity of a gene product non-targeted with the activity of a “305 ribosomal protein S3”, preferably being encoded by a gene comprising the nucleic acid sequence SEQ ID NO. 71325, preferably the coding region thereof, conferred the production of or the increase in hexadecadienoic acid compared with the wild type control.

The nucleic acid sequence of AvinDRAFT_{—}2754 from Azotobacter vinelandii, e.g. as shown in the respective line in column 5 of Table I, application no. 12, has been published in D. And the activity of the gene product thereof is the activity of acyl-CoA synthase.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “acyl-CoA synthase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said AvinDRAFT_{—}2754, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said AvinDRAFT_{—}2754, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said AvinDRAFT_{—}2754, or a functional equivalent or a homolog thereof as depicted in column 8 of Table II, application no. 12, preferably a homolog or functional equivalent as depicted in column 8 of Table II B, application no. 12, and being depicted in the same respective line as said AvinDRAFT_{—}2754, and preferably the activity is increased non-targeted, whereby the respective line discloses in column 7 the fine chemical 2-hydroxybehenic acid.

Accordingly, in one embodiment, the molecule which activity is to be increased in the process of the invention is the gene product with an activity as a “acyl-CoA synthase”, preferably it is the molecule of section (a) or (b) of this paragraph.

In particular, it was observed that in plants, especially in Arabidopsis thaliana, increasing or generating the activity of a gene product non-targeted with the activity of a “acyl-CoA synthase”, preferably being encoded by a gene comprising the nucleic acid sequence SEQ ID NO. 28040, preferably the coding region thereof, conferred the production of or the increase in 2-hydroxybehenic acid compared with the wild type control.

The nucleic acid sequence of AvinDRAFT_{—}2827 from Azotobacter vinelandii, e.g. as shown in the respective line in column 5 of Table I, application no. 12, has been published in D. And the activity of the gene product thereof is the activity of heat shock protein.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “heat shock protein”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said AvinDRAFT_{—}2827, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said AvinDRAFT_{—}2827, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said AvinDRAFT_{—}2827, or a functional equivalent or a homolog thereof as depicted in column 8 of Table II, application no. 12, preferably a homolog or functional equivalent as depicted in column 8 of Table II B, application no. 12, and being depicted in the same respective line as said AvinDRAFT_{—}2827, and preferably the activity is increased non-targeted, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “heat shock protein”, preferably it is the molecule of section (a) or (b) of this paragraph.

In particular, it was observed that in plants, especially in Arabidopsis thaliana, increasing or generating the activity of a gene product non-targeted with the activity of a “heat shock protein”, preferably being encoded by a gene comprising the nucleic acid sequence SEQ ID NO. 122948, preferably the coding region thereof, conferred the production of or the increase in 2-hydroxytetracosenoic acid (preferably 2-hydroxynervonic acid) compared with the wild type control.

The nucleic acid sequence of AvinDRAFT_{—}3253 from Azotobacter vinelandii, e.g. as shown in the respective line in column 5 of Table I, application no. 12, has been published in D. And the activity of the gene product thereof is the activity of oxidoreductase.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “oxidoreductase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said AvinDRAFT_{—}3253, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said AvinDRAFT_{—}3253, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said AvinDRAFT_{—}3253, or a functional equivalent or a homolog thereof as depicted in column 8 of Table II, application no. 12, preferably a homolog or functional equivalent as depicted in column 8 of Table II B, application no. 12, and being depicted in the same respective line as said AvinDRAFT_{—}3253, and preferably the activity is increased non-targeted, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 29500, preferably the coding region thereof, conferred the production of or the increase in 2-hydroxytetracosenoic acid (preferably 2-hydroxynervonic acid) compared with the wild type control.

The nucleic acid sequence of AvinDRAFT_{—}3629 from Azotobacter vinelandii, e.g. as shown in the respective line in column 5 of Table I, application no. 12, has been published in D. And the activity of the gene product thereof is the activity of recombinase A.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “recombinase A”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said AvinDRAFT_{—}3629, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said AvinDRAFT_{—}3629, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said AvinDRAFT_{—}3629, or a functional equivalent or a homolog thereof as depicted in column 8 of Table II, application no. 12, preferably a homolog or functional equivalent as depicted in column 8 of Table II B, application no. 12, and being depicted in the same respective line as said AvinDRAFT_{—}3629, and preferably the activity is increased non-targeted, whereby the respective line discloses in column 7 the fine chemical hexadecadienoic acid.

Accordingly, in one embodiment, the molecule which activity is to be increased in the process of the invention is the gene product with an activity as a “recombinase A”, preferably it is the molecule of section (a) or (b) of this paragraph.

In particular, it was observed that in plants, especially in Arabidopsis thaliana, increasing or generating the activity of a gene product non-targeted with the activity of a “recombinase A”, preferably being encoded by a gene comprising the nucleic acid sequence SEQ ID NO. 114231, preferably the coding region thereof, conferred the production of or the increase in hexadecadienoic acid compared with the wild type control.

The nucleic acid sequence of AVINDRAFT_{—}4128 from Azotobacter vinelandii, e.g. as shown in the respective line in column 5 of Table I, application no. 12, has been published in D. And the activity of the gene product thereof is the activity of ABC transporter component.

Accordingly, in one embodiment, the process of the present invention for producing lauric acid in a non-human organism, like a microorganism or a plant or a part thereof, comprises increasing or generating the activity of a gene product with the activity of a gene product conferring the activity of “ABC transporter component”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical lauric acid), application no. 12, preferably the coding region thereof, or a homolog or a fragment thereof, and being depicted in the same respective line as said AVINDRAFT_{—}4128, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said AVINDRAFT_{—}4128, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said AVINDRAFT_{—}4128, or a functional equivalent or a homolog thereof as depicted in column 8 of Table II, application no. 12, preferably a homolog or functional equivalent as depicted in column 8 of Table II B, application no. 12, and being depicted in the same respective line as said AVINDRAFT_{—}4128, and preferably the activity is increased non-targeted, whereby the respective line discloses in column 7 the fine chemical lauric acid.

Accordingly, in one embodiment, the molecule which activity is to be increased in the process of the invention is the gene product with an activity as a “ABC transporter component”, preferably it is the molecule of section (a) or (b) of this paragraph.

In particular, it was observed that in plants, especially in Arabidopsis thaliana, increasing or generating the activity of a gene product non-targeted with the activity of a “ABC transporter component”, preferably being encoded by a gene comprising the nucleic acid sequence SEQ ID NO. 72520, preferably the coding region thereof, conferred the production of or the increase in lauric acid compared with the wild type control.

The nucleic acid sequence of AvinDRAFT_{—}5103 from Azotobacter vinelandii, e.g. as shown in the respective line in column 5 of Table I, application no. 12, has been published in D. And the activity of the gene product thereof is the activity of hydrolase.

Accordingly, in one embodiment, the process of the present invention for producing 2-hydroxypalmitic acid in a non-human organism, like a microorganism or a plant or a part thereof, comprises increasing or generating the activity of a gene product with the activity of a gene product conferring the activity of “hydrolase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxypalmitic acid), application no. 12, preferably the coding region thereof, or a homolog or a fragment thereof, and being depicted in the same respective line as said AvinDRAFT_{—}5103, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said AvinDRAFT_{—}5103, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said AvinDRAFT_{—}5103, or a functional equivalent or a homolog thereof as depicted in column 8 of Table II, application no. 12, preferably a homolog or functional equivalent as depicted in column 8 of Table II B, application no. 12, and being depicted in the same respective line as said AvinDRAFT_{—}5103, and preferably the activity is increased non-targeted, whereby the respective line discloses in column 7 the fine chemical 2-hydroxypalmitic acid.

Accordingly, in one embodiment, the molecule which activity is to be increased in the process of the invention is the gene product with an activity as a “hydrolase”, preferably it is the molecule of section (a) or (b) of this paragraph.

In particular, it was observed that in plants, especially in Arabidopsis thaliana, increasing or generating the activity of a gene product non-targeted with the activity of a “hydrolase”, preferably being encoded by a gene comprising the nucleic acid sequence SEQ ID NO. 6510, preferably the coding region thereof, conferred the production of or the increase in 2-hydroxypalmitic acid compared with the wild type control.

The nucleic acid sequence of AvinDRAFT_{—}5246 from Azotobacter vinelandii, e.g. as shown in the respective line in column 5 of Table I, application no. 12, has been published in D. And the activity of the gene product thereof is the activity of glutamate-ammonia-ligase.

Accordingly, in one embodiment, the process of the present invention for producing 2-hydroxypalmitic acid in a non-human organism, like a microorganism or a plant or a part thereof, comprises increasing or generating the activity of a gene product with the activity of a gene product conferring the activity of “glutamate-ammonia-ligase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxypalmitic acid), application no. 12, preferably the coding region thereof, or a homolog or a fragment thereof, and being depicted in the same respective line as said AvinDRAFT_{—}5246, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said AvinDRAFT_{—}5246, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said AvinDRAFT_{—}5246, or a functional equivalent or a homolog thereof as depicted in column 8 of Table II, application no. 12, preferably a homolog or functional equivalent as depicted in column 8 of Table II B, application no. 12, and being depicted in the same respective line as said AvinDRAFT_{—}5246, and preferably the activity is increased non-targeted, whereby the respective line discloses in column 7 the fine chemical 2-hydroxypalmitic acid.

Accordingly, in one embodiment, the molecule which activity is to be increased in the process of the invention is the gene product with an activity as a “glutamate-ammonia-ligase”, preferably it is the molecule of section (a) or (b) of this paragraph.

In particular, it was observed that in plants, especially in Arabidopsis thaliana, increasing or generating the activity of a gene product non-targeted with the activity of a “glutamate-ammonia-ligase”, preferably being encoded by a gene comprising the nucleic acid sequence SEQ ID NO. 32037, preferably the coding region thereof, conferred the production of or the increase in 2-hydroxypalmitic acid compared with the wild type control.

The nucleic acid sequence of AvinDRAFT_{—}5292 from Azotobacter vinelandii, e.g. as shown in the respective line in column 5 of Table I, application no. 12, has been published in D. And the activity of the gene product thereof is the activity of fumarylacetoacetate hydrolase.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “fumarylacetoacetate hydrolase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said AvinDRAFT_{—}5292, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said AvinDRAFT_{—}5292, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said AvinDRAFT_{—}5292, or a functional equivalent or a homolog thereof as depicted in column 8 of Table II, application no. 12, preferably a homolog or functional equivalent as depicted in column 8 of Table II B, application no. 12, and being depicted in the same respective line as said AvinDRAFT_{—}5292, and preferably the activity is increased non-targeted, whereby the respective line discloses in column 7 the fine chemical 2-hydroxybehenic acid.

Accordingly, in one embodiment, the molecule which activity is to be increased in the process of the invention is the gene product with an activity as a “fumarylacetoacetate hydrolase”, preferably it is the molecule of section (a) or (b) of this paragraph.

In particular, it was observed that in plants, especially in Arabidopsis thaliana, increasing or generating the activity of a gene product non-targeted with the activity of a “fumarylacetoacetate hydrolase”, preferably being encoded by a gene comprising the nucleic acid sequence SEQ ID NO. 32308, preferably the coding region thereof, conferred the production of or the increase in 2-hydroxybehenic acid compared with the wild type control.

The nucleic acid sequence of AvinDRAFT_{—}5466 from Azotobacter vinelandii, e.g. as shown in the respective line in column 5 of Table I, application no. 12, has been published in D. And the activity of the gene product thereof is the activity of N-acetyl-gamma-glutamyl-phosphate reductase.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “N-acetyl-gamma-glutamyl-phosphate reductase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said AvinDRAFT_{—}5466, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said AvinDRAFT_{—}5466, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said AvinDRAFT_{—}5466, or a functional equivalent or a homolog thereof as depicted in column 8 of Table II, application no. 12, preferably a homolog or functional equivalent as depicted in column 8 of Table II B, application no. 12, and being depicted in the same respective line as said AvinDRAFT_{—}5466, and preferably the activity is increased non-targeted, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “N-acetyl-gamma-glutamyl-phosphate reductase”, preferably it is the molecule of section (a) or (b) of this paragraph.

In particular, it was observed that in plants, especially in Arabidopsis thaliana, increasing or generating the activity of a gene product non-targeted with the activity of a “N-acetyl-gamma-glutamyl-phosphate reductase”, preferably being encoded by a gene comprising the nucleic acid sequence SEQ ID NO. 123087, preferably the coding region thereof, conferred the production of or the increase in 2-hydroxytetracosenoic acid (preferably 2-hydroxynervonic acid) compared with the wild type control.

The nucleic acid sequence of AvinDRAFT_{—}5467 from Azotobacter vinelandii, e.g. as shown in the respective line in column 5 of Table I, application no. 12, has been published in D. And the activity of the gene product thereof is the activity of HesB/YadR/YfhF family protein.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “HesB/YadR/YfhF family protein”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said AvinDRAFT_{—}5467, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said AvinDRAFT_{—}5467, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said AvinDRAFT_{—}5467, or a functional equivalent or a homolog thereof as depicted in column 8 of Table II, application no. 12, preferably a homolog or functional equivalent as depicted in column 8 of Table II B, application no. 12, and being depicted in the same respective line as said AvinDRAFT_{—}5467, and preferably the activity is increased non-targeted, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “HesB/YadR/YfhF family protein”, preferably it is the molecule of section (a) or (b) of this paragraph.

In particular, it was observed that in plants, especially in Arabidopsis thaliana, increasing or generating the activity of a gene product non-targeted with the activity of a “HesB/YadR/YfhF family protein”, preferably being encoded by a gene comprising the nucleic acid sequence SEQ ID NO. 32648, preferably the coding region thereof, conferred the production of or the increase in 2-hydroxytetracosenoic acid (preferably 2-hydroxynervonic acid) compared with the wild type control.

The nucleic acid sequence of AvinDRAFT_{—}6700 from Azotobacter vinelandii, e.g. as shown in the respective line in column 5 of Table I, application no. 12, has been published in D. And the activity of the gene product thereof is the activity of purine nucleoside phosphorylase.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “purine nucleoside phosphorylase”, especially from Azotobacter vinelandii or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said AvinDRAFT_{—}6700, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said AvinDRAFT_{—}6700, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said AvinDRAFT_{—}6700, or a functional equivalent or a homolog thereof as depicted in column 8 of Table II, application no. 12, preferably a homolog or functional equivalent as depicted in column 8 of Table II B, application no. 12, and being depicted in the same respective line as said AvinDRAFT_{—}6700, and preferably the activity is increased non-targeted, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “purine nucleoside phosphorylase”, preferably it is the molecule of section (a) or (b) of this paragraph.

In particular, it was observed that in plants, especially in Arabidopsis thaliana, increasing or generating the activity of a gene product non-targeted with the activity of a “purine nucleoside phosphorylase”, preferably being encoded by a gene comprising the nucleic acid sequence SEQ ID NO. 34044, preferably the coding region thereof, conferred the production of or the increase in 2-hydroxytetracosenoic acid (preferably 2-hydroxynervonic acid) compared with the wild type control.

The nucleic acid sequence of B0050 from Escherichia coli, e.g. as shown in the respective line in column 5 of Table I, application no. 12, has been published in B. And the activity of the gene product thereof is the activity of b0050-protein.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “b0050-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said B0050, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B0050, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B0050, or a functional equivalent or a homolog thereof as depicted in column 8 of Table II, application no. 12, preferably a homolog or functional equivalent as depicted in column 8 of Table II B, application no. 12, and being depicted in the same respective line as said B0050, and preferably the activity is increased non-targeted, whereby the respective line discloses in column 7 the fine chemical 2-hydroxybehenic acid.

Accordingly, in one embodiment, the molecule which activity is to be increased in the process of the invention is the gene product with an activity as a “b0050-protein”, preferably it is the molecule of section (a) or (b) of this paragraph.

In particular, it was observed that in plants, especially in Arabidopsis thaliana, increasing or generating the activity of a gene product non-targeted with the activity of a “b0050-protein”, preferably being encoded by a gene comprising the nucleic acid sequence SEQ ID NO. 103958, preferably the coding region thereof, conferred the production of or the increase in 2-hydroxybehenic acid compared with the wild type control.

The nucleic acid sequence of B0050 from Escherichia coli, e.g. as shown in the respective line in column 5 of Table I, application no. 12, has been published in B. And the activity of the gene product thereof is the activity of b0050-protein.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “b0050-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said B0050, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B0050, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B0050, or a functional equivalent or a homolog thereof as depicted in column 8 of Table II, application no. 12, preferably a homolog or functional equivalent as depicted in column 8 of Table II B, application no. 12, and being depicted in the same respective line as said B0050, and preferably the activity is increased non-targeted, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “b0050-protein”, preferably it is the molecule of section (a) or (b) of this paragraph.

In particular, it was observed that in plants, especially in Arabidopsis thaliana, increasing or generating the activity of a gene product non-targeted with the activity of a “b0050-protein”, preferably being encoded by a gene comprising the nucleic acid sequence SEQ ID NO. 103958, preferably the coding region thereof, conferred the production of or the increase in 2-hydroxytetracosenoic acid (preferably 2-hydroxynervonic acid) compared with the wild type control.

The nucleic acid sequence of B0249 from Escherichia coli, e.g. as shown in the respective line in column 5 of Table I, application no. 12, has been published in B. And the activity of the gene product thereof is the activity of b0249-protein.

Accordingly, in one embodiment, the process of the present invention for producing cerebronic acid in a non-human organism, like a microorganism or a plant or a part thereof, comprises increasing or generating the activity of a gene product with the activity of a gene product conferring the activity of “b0249-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical cerebronic acid), application no. 12, preferably the coding region thereof, or a homolog or a fragment thereof, and being depicted in the same respective line as said B0249, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B0249, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B0249, or a functional equivalent or a homolog thereof as depicted in column 8 of Table II, application no. 12, preferably a homolog or functional equivalent as depicted in column 8 of Table II B, application no. 12, and being depicted in the same respective line as said B0249, and preferably the activity is increased non-targeted, whereby the respective line discloses in column 7 the fine chemical cerebronic acid.

Accordingly, in one embodiment, the molecule which activity is to be increased in the process of the invention is the gene product with an activity as a “b0249-protein”, preferably it is the molecule of section (a) or (b) of this paragraph.

In particular, it was observed that in plants, especially in Arabidopsis thaliana, increasing or generating the activity of a gene product non-targeted with the activity of a “b0249-protein”, preferably being encoded by a gene comprising the nucleic acid sequence SEQ ID NO. 123473, preferably the coding region thereof, conferred the production of or the increase in cerebronic acid compared with the wild type control.

The nucleic acid sequence of B0312 from Escherichia coli, e.g. as shown in the respective line in column 5 of Table I, application no. 12, has been published in B. And the activity of the gene product thereof is the activity of betaine aldehyde dehydrogenase.

Accordingly, in one embodiment, the process of the present invention for producing behenic acid in a non-human organism, like a microorganism or a plant or a part thereof, comprises increasing or generating the activity of a gene product with the activity of a gene product conferring the activity of “betaine aldehyde dehydrogenase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical behenic acid), application no. 12, preferably the coding region thereof, or a homolog or a fragment thereof, and being depicted in the same respective line as said B0312, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B0312, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B0312, or a functional equivalent or a homolog thereof as depicted in column 8 of Table II, application no. 12, preferably a homolog or functional equivalent as depicted in column 8 of Table II B, application no. 12, and being depicted in the same respective line as said B0312, and preferably the activity is increased non-targeted, whereby the respective line discloses in column 7 the fine chemical behenic acid.

Accordingly, in one embodiment, the molecule which activity is to be increased in the process of the invention is the gene product with an activity as a “betaine aldehyde dehydrogenase”, preferably it is the molecule of section (a) or (b) of this paragraph.

In particular, it was observed that in plants, especially in Arabidopsis thaliana, increasing or generating the activity of a gene product non-targeted with the activity of a “betaine aldehyde dehydrogenase”, preferably being encoded by a gene comprising the nucleic acid sequence SEQ ID NO. 121111, preferably the coding region thereof, conferred the production of or the increase in behenic acid compared with the wild type control.

The nucleic acid sequence of B0312 from Escherichia coli, e.g. as shown in the respective line in column 5 of Table I, application no. 12, has been published in B. And the activity of the gene product thereof is the activity of betaine aldehyde dehydrogenase.

Accordingly, in one embodiment, the process of the present invention for producing eicosanoic acid in a non-human organism, like a microorganism or a plant or a part thereof, comprises increasing or generating the activity of a gene product with the activity of a gene product conferring the activity of “betaine aldehyde dehydrogenase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical eicosanoic acid), application no. 12, preferably the coding region thereof, or a homolog or a fragment thereof, and being depicted in the same respective line as said B0312, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B0312, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B0312, or a functional equivalent or a homolog thereof as depicted in column 8 of Table II, application no. 12, preferably a homolog or functional equivalent as depicted in column 8 of Table II B, application no. 12, and being depicted in the same respective line as said B0312, and preferably the activity is increased non-targeted, whereby the respective line discloses in column 7 the fine chemical eicosanoic acid.

Accordingly, in one embodiment, the molecule which activity is to be increased in the process of the invention is the gene product with an activity as a “betaine aldehyde dehydrogenase”, preferably it is the molecule of section (a) or (b) of this paragraph.

In particular, it was observed that in plants, especially in Arabidopsis thaliana, increasing or generating the activity of a gene product non-targeted with the activity of a “betaine aldehyde dehydrogenase”, preferably being encoded by a gene comprising the nucleic acid sequence SEQ ID NO. 121111, preferably the coding region thereof, conferred the production of or the increase in eicosanoic acid compared with the wild type control.

The nucleic acid sequence of B0348 from Escherichia coli, e.g. as shown in the respective line in column 5 of Table I, application no. 12, has been published in B. And the activity of the gene product thereof is the activity of 2,3-dihydroxyphenylpropionate 1,2-dioxygenase.

Accordingly, in one embodiment, the process of the present invention for producing myristic acid in a non-human organism, like a microorganism or a plant or a part thereof, comprises increasing or generating the activity of a gene product with the activity of a gene product conferring the activity of “2,3-dihydroxyphenylpropionate 1,2-dioxygenase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical myristic acid), application no. 12, preferably the coding region thereof, or a homolog or a fragment thereof, and being depicted in the same respective line as said B0348, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B0348, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B0348, or a functional equivalent or a homolog thereof as depicted in column 8 of Table II, application no. 12, preferably a homolog or functional equivalent as depicted in column 8 of Table II B, application no. 12, and being depicted in the same respective line as said B0348, and preferably the activity is increased plastidic, whereby the respective line discloses in column 7 the fine chemical myristic acid.

Accordingly, in one embodiment, the molecule which activity is to be increased in the process of the invention is the gene product with an activity as a “2,3-dihydroxyphenylpropionate 1,2-dioxygenase”, preferably it is the molecule of section (a) or (b) of this paragraph.

In particular, it was observed that in plants, especially in Arabidopsis thaliana, increasing or generating the activity of a gene product plastidic with the activity of a “2,3-dihydroxyphenylpropionate 1,2-dioxygenase”, preferably being encoded by a gene comprising the nucleic acid sequence SEQ ID NO. 7269, preferably the coding region thereof, conferred the production of or the increase in myristic acid compared with the wild type control.

The nucleic acid sequence of B0348 from Escherichia coli, e.g. as shown in the respective line in column 5 of Table I, application no. 12, has been published in B. And the activity of the gene product thereof is the activity of 2,3-dihydroxyphenylpropionate 1,2-dioxygenase.

Accordingly, in one embodiment, the process of the present invention for producing oleic acid in a non-human organism, like a microorganism or a plant or a part thereof, comprises increasing or generating the activity of a gene product with the activity of a gene product conferring the activity of “2,3-dihydroxyphenylpropionate 1,2-dioxygenase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical oleic acid), application no. 12, preferably the coding region thereof, or a homolog or a fragment thereof, and being depicted in the same respective line as said B0348, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B0348, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B0348, or a functional equivalent or a homolog thereof as depicted in column 8 of Table II, application no. 12, preferably a homolog or functional equivalent as depicted in column 8 of Table II B, application no. 12, and being depicted in the same respective line as said B0348, and preferably the activity is increased plastidic, whereby the respective line discloses in column 7 the fine chemical oleic acid.

Accordingly, in one embodiment, the molecule which activity is to be increased in the process of the invention is the gene product with an activity as a “2,3-dihydroxyphenylpropionate 1,2-dioxygenase”, preferably it is the molecule of section (a) or (b) of this paragraph.

In particular, it was observed that in plants, especially in Arabidopsis thaliana, increasing or generating the activity of a gene product plastidic with the activity of a “2,3-dihydroxyphenylpropionate 1,2-dioxygenase”, preferably being encoded by a gene comprising the nucleic acid sequence SEQ ID NO. 7269, preferably the coding region thereof, conferred the production of or the increase in oleic acid compared with the wild type control.

The nucleic acid sequence of B0371 from Escherichia coli, e.g. as shown in the respective line in column 5 of Table I, application no. 12, has been published in B. And the activity of the gene product thereof is the activity of b0371-protein.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “b0371-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said B0371, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B0371, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B0371, or a functional equivalent or a homolog thereof as depicted in column 8 of Table II, application no. 12, preferably a homolog or functional equivalent as depicted in column 8 of Table II B, application no. 12, and being depicted in the same respective line as said B0371, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “b0371-protein”, preferably it is the molecule of section (a) or (b) of this paragraph.

In particular, it was observed that in plants, especially in Arabidopsis thaliana, increasing or generating the activity of a gene product non-targeted with the activity of a “b0371-protein”, preferably being encoded by a gene comprising the nucleic acid sequence SEQ ID NO. 121637, preferably the coding region thereof, conferred the production of or the increase in hexadecatrienoic acid compared with the wild type control.

The nucleic acid sequence of B0449 from Escherichia coli, e.g. as shown in the respective line in column 5 of Table I, application no. 12, has been published in B. And the activity of the gene product thereof is the activity of ATP-binding component of a transport system.

Accordingly, in one embodiment, the process of the present invention for producing cerebronic acid in a non-human organism, like a microorganism or a plant or a part thereof, comprises increasing or generating the activity of a gene product with the activity of a gene product conferring the activity of “ATP-binding component of a transport system”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical cerebronic acid), application no. 12, preferably the coding region thereof, or a homolog or a fragment thereof, and being depicted in the same respective line as said B0449, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B0449, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B0449, or a functional equivalent or a homolog thereof as depicted in column 8 of Table II, application no. 12, preferably a homolog or functional equivalent as depicted in column 8 of Table II B, application no. 12, and being depicted in the same respective line as said B0449, and preferably the activity is increased non-targeted, whereby the respective line discloses in column 7 the fine chemical cerebronic acid.

Accordingly, in one embodiment, the molecule which activity is to be increased in the process of the invention is the gene product with an activity as a “ATP-binding component of a transport system”, preferably it is the molecule of section (a) or (b) of this paragraph.

In particular, it was observed that in plants, especially in Arabidopsis thaliana, increasing or generating the activity of a gene product non-targeted with the activity of a “ATP-binding component of a transport system”, preferably being encoded by a gene comprising the nucleic acid sequence SEQ ID NO. 7333, preferably the coding region thereof, conferred the production of or the increase in cerebronic acid compared with the wild type control.

The nucleic acid sequence of B0449 from Escherichia coli, e.g. as shown in the respective line in column 5 of Table I, application no. 12, has been published in B. And the activity of the gene product thereof is the activity of ATP-binding component of a transport system.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “ATP-binding component of a transport system”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said B0449, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B0449, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B0449, or a functional equivalent or a homolog thereof as depicted in column 8 of Table II, application no. 12, preferably a homolog or functional equivalent as depicted in column 8 of Table II B, application no. 12, and being depicted in the same respective line as said B0449, and preferably the activity is increased non-targeted, whereby the respective line discloses in column 7 the fine chemical 2-hydroxybehenic acid.

Accordingly, in one embodiment, the molecule which activity is to be increased in the process of the invention is the gene product with an activity as a “ATP-binding component of a transport system”, preferably it is the molecule of section (a) or (b) of this paragraph.

In particular, it was observed that in plants, especially in Arabidopsis thaliana, increasing or generating the activity of a gene product non-targeted with the activity of a “ATP-binding component of a transport system”, preferably being encoded by a gene comprising the nucleic acid sequence SEQ ID NO. 7333, preferably the coding region thereof, conferred the production of or the increase in 2-hydroxybehenic acid compared with the wild type control.

The nucleic acid sequence of B0449 from Escherichia coli, e.g. as shown in the respective line in column 5 of Table I, application no. 12, has been published in B. And the activity of the gene product thereof is the activity of ATP-binding component of a transport system.

Accordingly, in one embodiment, the process of the present invention for producing 2-hydroxypalmitic acid in a non-human organism, like a microorganism or a plant or a part thereof, comprises increasing or generating the activity of a gene product with the activity of a gene product conferring the activity of “ATP-binding component of a transport system”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxypalmitic acid), application no. 12, preferably the coding region thereof, or a homolog or a fragment thereof, and being depicted in the same respective line as said B0449, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B0449, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B0449, or a functional equivalent or a homolog thereof as depicted in column 8 of Table II, application no. 12, preferably a homolog or functional equivalent as depicted in column 8 of Table II B, application no. 12, and being depicted in the same respective line as said B0449, and preferably the activity is increased non-targeted, whereby the respective line discloses in column 7 the fine chemical 2-hydroxypalmitic acid.

Accordingly, in one embodiment, the molecule which activity is to be increased in the process of the invention is the gene product with an activity as a “ATP-binding component of a transport system”, preferably it is the molecule of section (a) or (b) of this paragraph.

In particular, it was observed that in plants, especially in Arabidopsis thaliana, increasing or generating the activity of a gene product non-targeted with the activity of a “ATP-binding component of a transport system”, preferably being encoded by a gene comprising the nucleic acid sequence SEQ ID NO. 7333, preferably the coding region thereof, conferred the production of or the increase in 2-hydroxypalmitic acid compared with the wild type control.

The nucleic acid sequence of B0518 from Escherichia coli, e.g. as shown in the respective line in column 5 of Table I, application no. 12, has been published in B. And the activity of the gene product thereof is the activity of b0518-protein.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “b0518-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said B0518, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B0518, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B0518, or a functional equivalent or a homolog thereof as depicted in column 8 of Table II, application no. 12, preferably a homolog or functional equivalent as depicted in column 8 of Table II B, application no. 12, and being depicted in the same respective line as said B0518, and preferably the activity is increased non-targeted, whereby the respective line discloses in column 7 the fine chemical hexadecadienoic acid.

Accordingly, in one embodiment, the molecule which activity is to be increased in the process of the invention is the gene product with an activity as a “b0518-protein”, preferably it is the molecule of section (a) or (b) of this paragraph.

In particular, it was observed that in plants, especially in Arabidopsis thaliana, increasing or generating the activity of a gene product non-targeted with the activity of a “b0518-protein”, preferably being encoded by a gene comprising the nucleic acid sequence SEQ ID NO. 35936, preferably the coding region thereof, conferred the production of or the increase in hexadecadienoic acid compared with the wild type control.

The nucleic acid sequence of B0695 from Escherichia coli, e.g. as shown in the respective line in column 5 of Table I, application no. 12, has been published in B. And the activity of the gene product thereof is the activity of sensor histidine kinase.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “sensor histidine kinase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said B0695, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B0695, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B0695, or a functional equivalent or a homolog thereof as depicted in column 8 of Table II, application no. 12, preferably a homolog or functional equivalent as depicted in column 8 of Table II B, application no. 12, and being depicted in the same respective line as said B0695, and preferably the activity is increased non-targeted, whereby the respective line discloses in column 7 the fine chemical 2-hydroxybehenic acid.

Accordingly, in one embodiment, the molecule which activity is to be increased in the process of the invention is the gene product with an activity as a “sensor histidine kinase”, preferably it is the molecule of section (a) or (b) of this paragraph.

In particular, it was observed that in plants, especially in Arabidopsis thaliana, increasing or generating the activity of a gene product non-targeted with the activity of a “sensor histidine kinase”, preferably being encoded by a gene comprising the nucleic acid sequence SEQ ID NO. 93678, preferably the coding region thereof, conferred the production of or the increase in 2-hydroxybehenic acid compared with the wild type control.

The nucleic acid sequence of B0695 from Escherichia coli, e.g. as shown in the respective line in column 5 of Table I, application no. 12, has been published in B. And the activity of the gene product thereof is the activity of sensor histidine kinase.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “sensor histidine kinase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said B0695, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B0695, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B0695, or a functional equivalent or a homolog thereof as depicted in column 8 of Table II, application no. 12, preferably a homolog or functional equivalent as depicted in column 8 of Table II B, application no. 12, and being depicted in the same respective line as said B0695, and preferably the activity is increased non-targeted, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “sensor histidine kinase”, preferably it is the molecule of section (a) or (b) of this paragraph.

In particular, it was observed that in plants, especially in Arabidopsis thaliana, increasing or generating the activity of a gene product non-targeted with the activity of a “sensor histidine kinase”, preferably being encoded by a gene comprising the nucleic acid sequence SEQ ID NO. 93678, preferably the coding region thereof, conferred the production of or the increase in 2-hydroxytetracosenoic acid (preferably 2-hydroxynervonic acid) compared with the wild type control.

The nucleic acid sequence of B0720 from Escherichia coli, e.g. as shown in the respective line in column 5 of Table I, application no. 12, has been published in B. And the activity of the gene product thereof is the activity of citrate synthase.

Accordingly, in one embodiment, the process of the present invention for producing cerebronic acid in a non-human organism, like a microorganism or a plant or a part thereof, comprises increasing or generating the activity of a gene product with the activity of a gene product conferring the activity of “citrate synthase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical cerebronic acid), application no. 12, preferably the coding region thereof, or a homolog or a fragment thereof, and being depicted in the same respective line as said B0720, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B0720, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B0720, or a functional equivalent or a homolog thereof as depicted in column 8 of Table II, application no. 12, preferably a homolog or functional equivalent as depicted in column 8 of Table II B, application no. 12, and being depicted in the same respective line as said B0720, and preferably the activity is increased plastidic, whereby the respective line discloses in column 7 the fine chemical cerebronic acid.

Accordingly, in one embodiment, the molecule which activity is to be increased in the process of the invention is the gene product with an activity as a “citrate synthase”, preferably it is the molecule of section (a) or (b) of this paragraph.

In particular, it was observed that in plants, especially in Arabidopsis thaliana, increasing or generating the activity of a gene product plastidic with the activity of a “citrate synthase”, preferably being encoded by a gene comprising the nucleic acid sequence SEQ ID NO. 123498, preferably the coding region thereof, conferred the production of or the increase in cerebronic acid compared with the wild type control.

The nucleic acid sequence of B0801 from Escherichia coli, e.g. as shown in the respective line in column 5 of Table I, application no. 12, has been published in B. And the activity of the gene product thereof is the activity of b0801-protein.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “b0801-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said B0801, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B0801, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B0801, or a functional equivalent or a homolog thereof as depicted in column 8 of Table II, application no. 12, preferably a homolog or functional equivalent as depicted in column 8 of Table II B, application no. 12, and being depicted in the same respective line as said B0801, and preferably the activity is increased non-targeted, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “b0801-protein”, preferably it is the molecule of section (a) or (b) of this paragraph.

In particular, it was observed that in plants, especially in Arabidopsis thaliana, increasing or generating the activity of a gene product non-targeted with the activity of a “b0801-protein”, preferably being encoded by a gene comprising the nucleic acid sequence SEQ ID NO. 75807, preferably the coding region thereof, conferred the production of or the increase in 2-hydroxytetracosenoic acid (preferably 2-hydroxynervonic acid) compared with the wild type control.

The nucleic acid sequence of B1046 from Escherichia coli, e.g. as shown in the respective line in column 5 of Table I, application no. 12, has been published in B. And the activity of the gene product thereof is the activity of b1046-protein.

Accordingly, in one embodiment, the process of the present invention for producing cerebronic acid in a non-human organism, like a microorganism or a plant or a part thereof, comprises increasing or generating the activity of a gene product with the activity of a gene product conferring the activity of “b1046-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical cerebronic acid), application no. 12, preferably the coding region thereof, or a homolog or a fragment thereof, and being depicted in the same respective line as said B1046, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B1046, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B1046, or a functional equivalent or a homolog thereof as depicted in column 8 of Table II, application no. 12, preferably a homolog or functional equivalent as depicted in column 8 of Table II B, application no. 12, and being depicted in the same respective line as said B1046, and preferably the activity is increased plastidic, whereby the respective line discloses in column 7 the fine chemical cerebronic acid.

Accordingly, in one embodiment, the molecule which activity is to be increased in the process of the invention is the gene product with an activity as a “b1046-protein”, preferably it is the molecule of section (a) or (b) of this paragraph.

In particular, it was observed that in plants, especially in Arabidopsis thaliana, increasing or generating the activity of a gene product plastidic with the activity of a “b1046-protein”, preferably being encoded by a gene comprising the nucleic acid sequence SEQ ID NO. 124051, preferably the coding region thereof, conferred the production of or the increase in cerebronic acid compared with the wild type control.

The nucleic acid sequence of B1092 from Escherichia coli, e.g. as shown in the respective line in column 5 of Table I, application no. 12, has been published in B. And the activity of the gene product thereof is the activity of malonyl CoA-acyl carrier protein transacylase.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “malonyl CoA-acyl carrier protein transacylase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said B1092, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B1092, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B1092, or a functional equivalent or a homolog thereof as depicted in column 8 of Table II, application no. 12, preferably a homolog or functional equivalent as depicted in column 8 of Table II B, application no. 12, and being depicted in the same respective line as said B1092, and preferably the activity is increased plastidic, whereby the respective line discloses in column 7 the fine chemical hexadecatrienoic acid.

Accordingly, in one embodiment, the molecule which activity is to be increased in the process of the invention is the gene product with an activity as a “malonyl CoA-acyl carrier protein transacylase”, preferably it is the molecule of section (a) or (b) of this paragraph.

In particular, it was observed that in plants, especially in Arabidopsis thaliana, increasing or generating the activity of a gene product plastidic with the activity of a “malonyl CoA-acyl carrier protein transacylase”, preferably being encoded by a gene comprising the nucleic acid sequence SEQ ID NO. 124127, preferably the coding region thereof, conferred the production of or the increase in hexadecatrienoic acid compared with the wild type control.

The nucleic acid sequence of B1093 from Escherichia coli, e.g. as shown in the respective line in column 5 of Table I, application no. 12, has been published in B. And the activity of the gene product thereof is the activity of 3-oxoacyl-[acyl-carrier-protein] reductase.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “3-oxoacyl-[acyl-carrier-protein] reductase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said B1093, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B1093, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B1093, or a functional equivalent or a homolog thereof as depicted in column 8 of Table II, application no. 12, preferably a homolog or functional equivalent as depicted in column 8 of Table II B, application no. 12, and being depicted in the same respective line as said B1093, and preferably the activity is increased non-targeted, whereby the respective line discloses in column 7 the fine chemical hexadecadienoic acid.

Accordingly, in one embodiment, the molecule which activity is to be increased in the process of the invention is the gene product with an activity as a “3-oxoacyl-[acyl-carrier-protein] reductase”, preferably it is the molecule of section (a) or (b) of this paragraph.

In particular, it was observed that in plants, especially in Arabidopsis thaliana, increasing or generating the activity of a gene product non-targeted with the activity of a “3-oxoacyl-[acyl-carrier-protein] reductase”, preferably being encoded by a gene comprising the nucleic acid sequence SEQ ID NO. 118371, preferably the coding region thereof, conferred the production of or the increase in hexadecadienoic acid compared with the wild type control.

The nucleic acid sequence of B1093 from Escherichia coli, e.g. as shown in the respective line in column 5 of Table I, application no. 12, has been published in B. And the activity of the gene product thereof is the activity of 3-oxoacyl-[acyl-carrier-protein] reductase.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “3-oxoacyl-[acyl-carrier-protein] reductase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said B1093, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B1093, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B1093, or a functional equivalent or a homolog thereof as depicted in column 8 of Table II, application no. 12, preferably a homolog or functional equivalent as depicted in column 8 of Table II B, application no. 12, and being depicted in the same respective line as said B1093, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “3-oxoacyl-[acyl-carrier-protein] reductase”, preferably it is the molecule of section (a) or (b) of this paragraph.

In particular, it was observed that in plants, especially in Arabidopsis thaliana, increasing or generating the activity of a gene product non-targeted with the activity of a “3-oxoacyl-[acyl-carrier-protein] reductase”, preferably being encoded by a gene comprising the nucleic acid sequence SEQ ID NO. 118371, preferably the coding region thereof, conferred the production of or the increase in hexadecatrienoic acid compared with the wild type control.

The nucleic acid sequence of B1095 from Escherichia coli, e.g. as shown in the respective line in column 5 of Table I, application no. 12, has been published in B. And the activity of the gene product thereof is the activity of 3-oxoacyl-[acyl-carrier-protein] synthase.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “3-oxoacyl-[acyl-carrier-protein] synthase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said B1095, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B1095, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B1095, or a functional equivalent or a homolog thereof as depicted in column 8 of Table II, application no. 12, preferably a homolog or functional equivalent as depicted in column 8 of Table II B, application no. 12, and being depicted in the same respective line as said B1095, and preferably the activity is increased plastidic, whereby the respective line discloses in column 7 the fine chemical hexadecatrienoic acid.

Accordingly, in one embodiment, the molecule which activity is to be increased in the process of the invention is the gene product with an activity as a “3-oxoacyl-[acyl-carrier-protein] synthase”, preferably it is the molecule of section (a) or (b) of this paragraph.

In particular, it was observed that in plants, especially in Arabidopsis thaliana, increasing or generating the activity of a gene product plastidic with the activity of a “3-oxoacyl-[acyl-carrier-protein] synthase”, preferably being encoded by a gene comprising the nucleic acid sequence SEQ ID NO. 114552, preferably the coding region thereof, conferred the production of or the increase in hexadecatrienoic acid compared with the wild type control.

The nucleic acid sequence of B1095 from Escherichia coli, e.g. as shown in the respective line in column 5 of Table I, application no. 12, has been published in B. And the activity of the gene product thereof is the activity of 3-oxoacyl-[acyl-carrier-protein] synthase.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “3-oxoacyl-[acyl-carrier-protein] synthase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said B1095, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B1095, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B1095, or a functional equivalent or a homolog thereof as depicted in column 8 of Table II, application no. 12, preferably a homolog or functional equivalent as depicted in column 8 of Table II B, application no. 12, and being depicted in the same respective line as said B1095, and preferably the activity is increased plastidic, whereby the respective line discloses in column 7 the fine chemical 2-hydroxybehenic acid.

Accordingly, in one embodiment, the molecule which activity is to be increased in the process of the invention is the gene product with an activity as a “3-oxoacyl-[acyl-carrier-protein] synthase”, preferably it is the molecule of section (a) or (b) of this paragraph.

In particular, it was observed that in plants, especially in Arabidopsis thaliana, increasing or generating the activity of a gene product plastidic with the activity of a “3-oxoacyl-[acyl-carrier-protein] synthase”, preferably being encoded by a gene comprising the nucleic acid sequence SEQ ID NO. 114552, preferably the coding region thereof, conferred the production of or the increase in 2-hydroxybehenic acid compared with the wild type control.

The nucleic acid sequence of B1186 from Escherichia coli, e.g. as shown in the respective line in column 5 of Table I, application no. 12, has been published in B. And the activity of the gene product thereof is the activity of sodium/proton antiporter.

Accordingly, in one embodiment, the process of the present invention for producing oleic acid in a non-human organism, like a microorganism or a plant or a part thereof, comprises increasing or generating the activity of a gene product with the activity of a gene product conferring the activity of “sodium/proton antiporter”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical oleic acid), application no. 12, preferably the coding region thereof, or a homolog or a fragment thereof, and being depicted in the same respective line as said B1186, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B1186, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B1186, or a functional equivalent or a homolog thereof as depicted in column 8 of Table II, application no. 12, preferably a homolog or functional equivalent as depicted in column 8 of Table II B, application no. 12, and being depicted in the same respective line as said B1186, and preferably the activity is increased non-targeted, whereby the respective line discloses in column 7 the fine chemical oleic acid.

Accordingly, in one embodiment, the molecule which activity is to be increased in the process of the invention is the gene product with an activity as a “sodium/proton antiporter”, preferably it is the molecule of section (a) or (b) of this paragraph.

In particular, it was observed that in plants, especially in Arabidopsis thaliana, increasing or generating the activity of a gene product non-targeted with the activity of a “sodium/proton antiporter”, preferably being encoded by a gene comprising the nucleic acid sequence SEQ ID NO. 37400, preferably the coding region thereof, conferred the production of or the increase in oleic acid compared with the wild type control.

The nucleic acid sequence of B1186 from Escherichia coli, e.g. as shown in the respective line in column 5 of Table I, application no. 12, has been published in B. And the activity of the gene product thereof is the activity of sodium/proton antiporter.

Accordingly, in one embodiment, the process of the present invention for producing 2-hydroxypalmitic acid in a non-human organism, like a microorganism or a plant or a part thereof, comprises increasing or generating the activity of a gene product with the activity of a gene product conferring the activity of “sodium/proton antiporter”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxypalmitic acid), application no. 12, preferably the coding region thereof, or a homolog or a fragment thereof, and being depicted in the same respective line as said B1186, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B1186, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B1186, or a functional equivalent or a homolog thereof as depicted in column 8 of Table II, application no. 12, preferably a homolog or functional equivalent as depicted in column 8 of Table II B, application no. 12, and being depicted in the same respective line as said B1186, and preferably the activity is increased non-targeted, whereby the respective line discloses in column 7 the fine chemical 2-hydroxypalmitic acid.

Accordingly, in one embodiment, the molecule which activity is to be increased in the process of the invention is the gene product with an activity as a “sodium/proton antiporter”, preferably it is the molecule of section (a) or (b) of this paragraph.

In particular, it was observed that in plants, especially in Arabidopsis thaliana, increasing or generating the activity of a gene product non-targeted with the activity of a “sodium/proton antiporter”, preferably being encoded by a gene comprising the nucleic acid sequence SEQ ID NO. 37400, preferably the coding region thereof, conferred the production of or the increase in 2-hydroxypalmitic acid compared with the wild type control.

The nucleic acid sequence of B1209 from Escherichia coli, e.g. as shown in the respective line in column 5 of Table I, application no. 12, has been published in B. And the activity of the gene product thereof is the activity of outer-membrane lipoprotein precursor.

Accordingly, in one embodiment, the process of the present invention for producing behenic acid in a non-human organism, like a microorganism or a plant or a part thereof, comprises increasing or generating the activity of a gene product with the activity of a gene product conferring the activity of “outer-membrane lipoprotein precursor”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical behenic acid), application no. 12, preferably the coding region thereof, or a homolog or a fragment thereof, and being depicted in the same respective line as said B1209, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B1209, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B1209, or a functional equivalent or a homolog thereof as depicted in column 8 of Table II, application no. 12, preferably a homolog or functional equivalent as depicted in column 8 of Table II B, application no. 12, and being depicted in the same respective line as said B1209, and preferably the activity is increased plastidic, whereby the respective line discloses in column 7 the fine chemical behenic acid.

Accordingly, in one embodiment, the molecule which activity is to be increased in the process of the invention is the gene product with an activity as a “outer-membrane lipoprotein precursor”, preferably it is the molecule of section (a) or (b) of this paragraph.

In particular, it was observed that in plants, especially in Arabidopsis thaliana, increasing or generating the activity of a gene product plastidic with the activity of a “outer-membrane lipoprotein precursor”, preferably being encoded by a gene comprising the nucleic acid sequence SEQ ID NO. 124509, preferably the coding region thereof, conferred the production of or the increase in behenic acid compared with the wild type control.

The nucleic acid sequence of B1249 from Escherichia coli, e.g. as shown in the respective line in column 5 of Table I, application no. 12, has been published in B. And the activity of the gene product thereof is the activity of cardiolipin synthase.

Accordingly, in one embodiment, the process of the present invention for producing behenic acid in a non-human organism, like a microorganism or a plant or a part thereof, comprises increasing or generating the activity of a gene product with the activity of a gene product conferring the activity of “cardiolipin synthase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical behenic acid), application no. 12, preferably the coding region thereof, or a homolog or a fragment thereof, and being depicted in the same respective line as said B1249, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B1249, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B1249, or a functional equivalent or a homolog thereof as depicted in column 8 of Table II, application no. 12, preferably a homolog or functional equivalent as depicted in column 8 of Table II B, application no. 12, and being depicted in the same respective line as said B1249, and preferably the activity is increased plastidic, whereby the respective line discloses in column 7 the fine chemical behenic acid.

Accordingly, in one embodiment, the molecule which activity is to be increased in the process of the invention is the gene product with an activity as a “cardiolipin synthase”, preferably it is the molecule of section (a) or (b) of this paragraph.

In particular, it was observed that in plants, especially in Arabidopsis thaliana, increasing or generating the activity of a gene product plastidic with the activity of a “cardiolipin synthase”, preferably being encoded by a gene comprising the nucleic acid sequence SEQ ID NO. 98778, preferably the coding region thereof, conferred the production of or the increase in behenic acid compared with the wild type control.

The nucleic acid sequence of B1249 from Escherichia coli, e.g. as shown in the respective line in column 5 of Table I, application no. 12, has been published in B. And the activity of the gene product thereof is the activity of cardiolipin synthase.

Accordingly, in one embodiment, the process of the present invention for producing heptadecanoic acid in a non-human organism, like a microorganism or a plant or a part thereof, comprises increasing or generating the activity of a gene product with the activity of a gene product conferring the activity of “cardiolipin synthase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical heptadecanoic acid), application no. 12, preferably the coding region thereof, or a homolog or a fragment thereof, and being depicted in the same respective line as said B1249, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B1249, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B1249, or a functional equivalent or a homolog thereof as depicted in column 8 of Table II, application no. 12, preferably a homolog or functional equivalent as depicted in column 8 of Table II B, application no. 12, and being depicted in the same respective line as said B1249, and preferably the activity is increased plastidic, whereby the respective line discloses in column 7 the fine chemical heptadecanoic acid.

Accordingly, in one embodiment, the molecule which activity is to be increased in the process of the invention is the gene product with an activity as a “cardiolipin synthase”, preferably it is the molecule of section (a) or (b) of this paragraph.

In particular, it was observed that in plants, especially in Arabidopsis thaliana, increasing or generating the activity of a gene product plastidic with the activity of a “cardiolipin synthase”, preferably being encoded by a gene comprising the nucleic acid sequence SEQ ID NO. 98778, preferably the coding region thereof, conferred the production of or the increase in heptadecanoic acid compared with the wild type control.

The nucleic acid sequence of B1346 from Escherichia coli, e.g. as shown in the respective line in column 5 of Table I, application no. 12, has been published in B. And the activity of the gene product thereof is the activity of b1346-protein.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “b1346-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said B1346, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B1346, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B1346, or a functional equivalent or a homolog thereof as depicted in column 8 of Table II, application no. 12, preferably a homolog or functional equivalent as depicted in column 8 of Table II B, application no. 12, and being depicted in the same respective line as said B1346, and preferably the activity is increased plastidic, whereby the respective line discloses in column 7 the fine chemical hexadecatrienoic acid.

Accordingly, in one embodiment, the molecule which activity is to be increased in the process of the invention is the gene product with an activity as a “b1346-protein”, preferably it is the molecule of section (a) or (b) of this paragraph.

In particular, it was observed that in plants, especially in Arabidopsis thaliana, increasing or generating the activity of a gene product plastidic with the activity of a “b1346-protein”, preferably being encoded by a gene comprising the nucleic acid sequence SEQ ID NO. 99188, preferably the coding region thereof, conferred the production of or the increase in hexadecatrienoic acid compared with the wild type control.

The nucleic acid sequence of B1356 from Escherichia coli, e.g. as shown in the respective line in column 5 of Table I, application no. 12, has been published in B. And the activity of the gene product thereof is the activity of phage-related repressor protein.

Accordingly, in one embodiment, the process of the present invention for producing lauric acid in a non-human organism, like a microorganism or a plant or a part thereof, comprises increasing or generating the activity of a gene product with the activity of a gene product conferring the activity of “phage-related repressor protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical lauric acid), application no. 12, preferably the coding region thereof, or a homolog or a fragment thereof, and being depicted in the same respective line as said B1356, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B1356, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B1356, or a functional equivalent or a homolog thereof as depicted in column 8 of Table II, application no. 12, preferably a homolog or functional equivalent as depicted in column 8 of Table II B, application no. 12, and being depicted in the same respective line as said B1356, and preferably the activity is increased non-targeted, whereby the respective line discloses in column 7 the fine chemical lauric acid.

Accordingly, in one embodiment, the molecule which activity is to be increased in the process of the invention is the gene product with an activity as a “phage-related repressor protein”, preferably it is the molecule of section (a) or (b) of this paragraph.

In particular, it was observed that in plants, especially in Arabidopsis thaliana, increasing or generating the activity of a gene product non-targeted with the activity of a “phage-related repressor protein”, preferably being encoded by a gene comprising the nucleic acid sequence SEQ ID NO. 124557, preferably the coding region thereof, conferred the production of or the increase in lauric acid compared with the wild type control.

The nucleic acid sequence of B1431 from Escherichia coli, e.g. as shown in the respective line in column 5 of Table I, application no. 12, has been published in B. And the activity of the gene product thereof is the activity of lipoprotein precursor.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “lipoprotein precursor”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said B1431, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B1431, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B1431, or a functional equivalent or a homolog thereof as depicted in column 8 of Table II, application no. 12, preferably a homolog or functional equivalent as depicted in column 8 of Table II B, application no. 12, and being depicted in the same respective line as said B1431, and preferably the activity is increased plastidic, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “lipoprotein precursor”, preferably it is the molecule of section (a) or (b) of this paragraph.

In particular, it was observed that in plants, especially in Arabidopsis thaliana, increasing or generating the activity of a gene product plastidic with the activity of a “lipoprotein precursor”, preferably being encoded by a gene comprising the nucleic acid sequence SEQ ID NO. 38266, preferably the coding region thereof, conferred the production of or the increase in 2-hydroxytetracosenoic acid (preferably 2-hydroxynervonic acid) compared with the wild type control.

The nucleic acid sequence of B1470 from Escherichia coli, e.g. as shown in the respective line in column 5 of Table I, application no. 12, has been published in B. And the activity of the gene product thereof is the activity of b1470-protein.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “b1470-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said B1470, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B1470, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B1470, or a functional equivalent or a homolog thereof as depicted in column 8 of Table II, application no. 12, preferably a homolog or functional equivalent as depicted in column 8 of Table II B, application no. 12, and being depicted in the same respective line as said B1470, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “b1470-protein”, preferably it is the molecule of section (a) or (b) of this paragraph.

In particular, it was observed that in plants, especially in Arabidopsis thaliana, increasing or generating the activity of a gene product non-targeted with the activity of a “b1470-protein”, preferably being encoded by a gene comprising the nucleic acid sequence SEQ ID NO. 115056, preferably the coding region thereof, conferred the production of or the increase in hexadecatrienoic acid compared with the wild type control.

The nucleic acid sequence of B1479 from Escherichia coli, e.g. as shown in the respective line in column 5 of Table I, application no. 12, has been published in B. And the activity of the gene product thereof is the activity of NADP-dependent malic enzyme.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “NADP-dependent malic enzyme”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said B1479, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B1479, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B1479, or a functional equivalent or a homolog thereof as depicted in column 8 of Table II, application no. 12, preferably a homolog or functional equivalent as depicted in column 8 of Table II B, application no. 12, and being depicted in the same respective line as said B1479, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “NADP-dependent malic enzyme”, preferably it is the molecule of section (a) or (b) of this paragraph.

In particular, it was observed that in plants, especially in Arabidopsis thaliana, increasing or generating the activity of a gene product non-targeted with the activity of a “NADP-dependent malic enzyme”, preferably being encoded by a gene comprising the nucleic acid sequence SEQ ID NO. 77774, preferably the coding region thereof, conferred the production of or the increase in hexadecatrienoic acid compared with the wild type control.

The nucleic acid sequence of B1522 from Escherichia coli, e.g. as shown in the respective line in column 5 of Table I, application no. 12, has been published in B. And the activity of the gene product thereof is the activity of b1522-protein.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “b1522-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said B1522, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B1522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B1522, or a functional equivalent or a homolog thereof as depicted in column 8 of Table II, application no. 12, preferably a homolog or functional equivalent as depicted in column 8 of Table II B, application no. 12, and being depicted in the same respective line as said B1522, and preferably the activity is increased non-targeted, whereby the respective line discloses in column 7 the fine chemical 2-hydroxybehenic acid.

Accordingly, in one embodiment, the molecule which activity is to be increased in the process of the invention is the gene product with an activity as a “b1522-protein”, preferably it is the molecule of section (a) or (b) of this paragraph.

In particular, it was observed that in plants, especially in Arabidopsis thaliana, increasing or generating the activity of a gene product non-targeted with the activity of a “b1522-protein”, preferably being encoded by a gene comprising the nucleic acid sequence SEQ ID NO. 7947, preferably the coding region thereof, conferred the production of or the increase in 2-hydroxybehenic acid compared with the wild type control.

The nucleic acid sequence of B1597 from Escherichia coli, e.g. as shown in the respective line in column 5 of Table I, application no. 12, has been published in B. And the activity of the gene product thereof is the activity of acid shock protein.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “acid shock protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said B1597, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B1597, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B1597, or a functional equivalent or a homolog thereof as depicted in column 8 of Table II, application no. 12, preferably a homolog or functional equivalent as depicted in column 8 of Table II B, application no. 12, and being depicted in the same respective line as said B1597, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “acid shock protein”, preferably it is the molecule of section (a) or (b) of this paragraph.

In particular, it was observed that in plants, especially in Arabidopsis thaliana, increasing or generating the activity of a gene product non-targeted with the activity of a “acid shock protein”, preferably being encoded by a gene comprising the nucleic acid sequence SEQ ID NO. 38300, preferably the coding region thereof, conferred the production of or the increase in hexadecatrienoic acid compared with the wild type control.

The nucleic acid sequence of B1601 from Escherichia coli, e.g. as shown in the respective line in column 5 of Table I, application no. 12, has been published in B. And the activity of the gene product thereof is the activity of transport protein.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “transport protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said B1601, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B1601, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B1601, or a functional equivalent or a homolog thereof as depicted in column 8 of Table II, application no. 12, preferably a homolog or functional equivalent as depicted in column 8 of Table II B, application no. 12, and being depicted in the same respective line as said B1601, and preferably the activity is increased non-targeted, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “transport protein”, preferably it is the molecule of section (a) or (b) of this paragraph.

In particular, it was observed that in plants, especially in Arabidopsis thaliana, increasing or generating the activity of a gene product non-targeted with the activity of a “transport protein”, preferably being encoded by a gene comprising the nucleic acid sequence SEQ ID NO. 7992, preferably the coding region thereof, conferred the production of or the increase in 2-hydroxytetracosenoic acid (preferably 2-hydroxynervonic acid) compared with the wild type control.

The nucleic acid sequence of B1621 from Escherichia coli, e.g. as shown in the respective line in column 5 of Table I, application no. 12, has been published in B. And the activity of the gene product thereof is the activity of phosphotransferase system component.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “phosphotransferase system component”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said B1621, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B1621, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B1621, or a functional equivalent or a homolog thereof as depicted in column 8 of Table II, application no. 12, preferably a homolog or functional equivalent as depicted in column 8 of Table II B, application no. 12, and being depicted in the same respective line as said B1621, and preferably the activity is increased plastidic, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “phosphotransferase system component”, preferably it is the molecule of section (a) or (b) of this paragraph.

In particular, it was observed that in plants, especially in Arabidopsis thaliana, increasing or generating the activity of a gene product plastidic with the activity of a “phosphotransferase system component”, preferably being encoded by a gene comprising the nucleic acid sequence SEQ ID NO. 124571, preferably the coding region thereof, conferred the production of or the increase in 2-hydroxytetracosenoic acid (preferably 2-hydroxynervonic acid) compared with the wild type control.

The nucleic acid sequence of B1627 from Escherichia coli, e.g. as shown in the respective line in column 5 of Table I, application no. 12, has been published in B. And the activity of the gene product thereof is the activity of electron transport complex protein.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “electron transport complex protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said B1627, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B1627, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B1627, or a functional equivalent or a homolog thereof as depicted in column 8 of Table II, application no. 12, preferably a homolog or functional equivalent as depicted in column 8 of Table II B, application no. 12, and being depicted in the same respective line as said B1627, and preferably the activity is increased plastidic, whereby the respective line discloses in column 7 the fine chemical 2-hydroxybehenic acid.

Accordingly, in one embodiment, the molecule which activity is to be increased in the process of the invention is the gene product with an activity as a “electron transport complex protein”, preferably it is the molecule of section (a) or (b) of this paragraph.

In particular, it was observed that in plants, especially in Arabidopsis thaliana, increasing or generating the activity of a gene product plastidic with the activity of a “electron transport complex protein”, preferably being encoded by a gene comprising the nucleic acid sequence SEQ ID NO. 38345, preferably the coding region thereof, conferred the production of or the increase in 2-hydroxybehenic acid compared with the wild type control.

The nucleic acid sequence of B1636 from Escherichia coli, e.g. as shown in the respective line in column 5 of Table I, application no. 12, has been published in B. And the activity of the gene product thereof is the activity of pyridoxamine kinase.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “pyridoxamine kinase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said B1636, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B1636, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B1636, or a functional equivalent or a homolog thereof as depicted in column 8 of Table II, application no. 12, preferably a homolog or functional equivalent as depicted in column 8 of Table II B, application no. 12, and being depicted in the same respective line as said B1636, and preferably the activity is increased plastidic, whereby the respective line discloses in column 7 the fine chemical hexadecatrienoic acid.

Accordingly, in one embodiment, the molecule which activity is to be increased in the process of the invention is the gene product with an activity as a “pyridoxamine kinase”, preferably it is the molecule of section (a) or (b) of this paragraph.

In particular, it was observed that in plants, especially in Arabidopsis thaliana, increasing or generating the activity of a gene product plastidic with the activity of a “pyridoxamine kinase”, preferably being encoded by a gene comprising the nucleic acid sequence SEQ ID NO. 124805, preferably the coding region thereof, conferred the production of or the increase in hexadecatrienoic acid compared with the wild type control.

The nucleic acid sequence of B1670 from Escherichia coli, e.g. as shown in the respective line in column 5 of Table I, application no. 12, has been published in B. And the activity of the gene product thereof is the activity of b1670-protein.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “b1670-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said B1670, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B1670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B1670, or a functional equivalent or a homolog thereof as depicted in column 8 of Table II, application no. 12, preferably a homolog or functional equivalent as depicted in column 8 of Table II B, application no. 12, and being depicted in the same respective line as said B1670, and preferably the activity is increased non-targeted, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “b1670-protein”, preferably it is the molecule of section (a) or (b) of this paragraph.

In particular, it was observed that in plants, especially in Arabidopsis thaliana, increasing or generating the activity of a gene product non-targeted with the activity of a “b1670-protein”, preferably being encoded by a gene comprising the nucleic acid sequence SEQ ID NO. 78753, preferably the coding region thereof, conferred the production of or the increase in 2-hydroxytetracosenoic acid (preferably 2-hydroxynervonic acid) compared with the wild type control.

The nucleic acid sequence of B1703 from Escherichia coli, e.g. as shown in the respective line in column 5 of Table I, application no. 12, has been published in B. And the activity of the gene product thereof is the activity of b1703-protein.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “b1703-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said B1703, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B1703, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B1703, or a functional equivalent or a homolog thereof as depicted in column 8 of Table II, application no. 12, preferably a homolog or functional equivalent as depicted in column 8 of Table II B, application no. 12, and being depicted in the same respective line as said B1703, and preferably the activity is increased non-targeted, whereby the respective line discloses in column 7 the fine chemical hexadecadienoic acid.

Accordingly, in one embodiment, the molecule which activity is to be increased in the process of the invention is the gene product with an activity as a “b1703-protein”, preferably it is the molecule of section (a) or (b) of this paragraph.

In particular, it was observed that in plants, especially in Arabidopsis thaliana, increasing or generating the activity of a gene product non-targeted with the activity of a “b1703-protein”, preferably being encoded by a gene comprising the nucleic acid sequence SEQ ID NO. 124907, preferably the coding region thereof, conferred the production of or the increase in hexadecadienoic acid compared with the wild type control.

The nucleic acid sequence of B1703 from Escherichia coli, e.g. as shown in the respective line in column 5 of Table I, application no. 12, has been published in B. And the activity of the gene product thereof is the activity of b1703-protein.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “b1703-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said B1703, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B1703, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B1703, or a functional equivalent or a homolog thereof as depicted in column 8 of Table II, application no. 12, preferably a homolog or functional equivalent as depicted in column 8 of Table II B, application no. 12, and being depicted in the same respective line as said B1703, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “b1703-protein”, preferably it is the molecule of section (a) or (b) of this paragraph.

In particular, it was observed that in plants, especially in Arabidopsis thaliana, increasing or generating the activity of a gene product non-targeted with the activity of a “b1703-protein”, preferably being encoded by a gene comprising the nucleic acid sequence SEQ ID NO. 124907, preferably the coding region thereof, conferred the production of or the increase in hexadecatrienoic acid compared with the wild type control.

The nucleic acid sequence of B1791 from Escherichia coli, e.g. as shown in the respective line in column 5 of Table I, application no. 12, has been published in B. And the activity of the gene product thereof is the activity of transport protein.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “transport protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said B1791, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B1791, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B1791, or a functional equivalent or a homolog thereof as depicted in column 8 of Table II, application no. 12, preferably a homolog or functional equivalent as depicted in column 8 of Table II B, application no. 12, and being depicted in the same respective line as said B1791, and preferably the activity is increased non-targeted, whereby the respective line discloses in column 7 the fine chemical hexadecadienoic acid.

Accordingly, in one embodiment, the molecule which activity is to be increased in the process of the invention is the gene product with an activity as a “transport protein”, preferably it is the molecule of section (a) or (b) of this paragraph.

In particular, it was observed that in plants, especially in Arabidopsis thaliana, increasing or generating the activity of a gene product non-targeted with the activity of a “transport protein”, preferably being encoded by a gene comprising the nucleic acid sequence SEQ ID NO. 78883, preferably the coding region thereof, conferred the production of or the increase in hexadecadienoic acid compared with the wild type control.

The nucleic acid sequence of B1837 from Escherichia coli, e.g. as shown in the respective line in column 5 of Table I, application no. 12, has been published in B. And the activity of the gene product thereof is the activity of b1837-protein.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “b1837-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said B1837, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B1837, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B1837, or a functional equivalent or a homolog thereof as depicted in column 8 of Table II, application no. 12, preferably a homolog or functional equivalent as depicted in column 8 of Table II B, application no. 12, and being depicted in the same respective line as said B1837, and preferably the activity is increased non-targeted, whereby the respective line discloses in column 7 the fine chemical hexadecadienoic acid.

Accordingly, in one embodiment, the molecule which activity is to be increased in the process of the invention is the gene product with an activity as a “b1837-protein”, preferably it is the molecule of section (a) or (b) of this paragraph.

In particular, it was observed that in plants, especially in Arabidopsis thaliana, increasing or generating the activity of a gene product non-targeted with the activity of a “b1837-protein”, preferably being encoded by a gene comprising the nucleic acid sequence SEQ ID NO. 78953, preferably the coding region thereof, conferred the production of or the increase in hexadecadienoic acid compared with the wild type control.

The nucleic acid sequence of B1837 from Escherichia coli, e.g. as shown in the respective line in column 5 of Table I, application no. 12, has been published in B. And the activity of the gene product thereof is the activity of b1837-protein.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “b1837-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said B1837, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B1837, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B1837, or a functional equivalent or a homolog thereof as depicted in column 8 of Table II, application no. 12, preferably a homolog or functional equivalent as depicted in column 8 of Table II B, application no. 12, and being depicted in the same respective line as said B1837, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “b1837-protein”, preferably it is the molecule of section (a) or (b) of this paragraph.

In particular, it was observed that in plants, especially in Arabidopsis thaliana, increasing or generating the activity of a gene product non-targeted with the activity of a “b1837-protein”, preferably being encoded by a gene comprising the nucleic acid sequence SEQ ID NO. 78953, preferably the coding region thereof, conferred the production of or the increase in hexadecatrienoic acid compared with the wild type control.

The nucleic acid sequence of B2032 from Escherichia coli, e.g. as shown in the respective line in column 5 of Table I, application no. 12, has been published in B. And the activity of the gene product thereof is the activity of b2032-protein.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “b2032-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said B2032, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B2032, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B2032, or a functional equivalent or a homolog thereof as depicted in column 8 of Table II, application no. 12, preferably a homolog or functional equivalent as depicted in column 8 of Table II B, application no. 12, and being depicted in the same respective line as said B2032, and preferably the activity is increased non-targeted, whereby the respective line discloses in column 7 the fine chemical hexadecadienoic acid.

Accordingly, in one embodiment, the molecule which activity is to be increased in the process of the invention is the gene product with an activity as a “b2032-protein”, preferably it is the molecule of section (a) or (b) of this paragraph.

In particular, it was observed that in plants, especially in Arabidopsis thaliana, increasing or generating the activity of a gene product non-targeted with the activity of a “b2032-protein”, preferably being encoded by a gene comprising the nucleic acid sequence SEQ ID NO. 8920, preferably the coding region thereof, conferred the production of or the increase in hexadecadienoic acid compared with the wild type control.

The nucleic acid sequence of B2099 from Escherichia coli, e.g. as shown in the respective line in column 5 of Table I, application no. 12, has been published in B. And the activity of the gene product thereof is the activity of b2099-protein.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “b2099-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said B2099, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B2099, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B2099, or a functional equivalent or a homolog thereof as depicted in column 8 of Table II, application no. 12, preferably a homolog or functional equivalent as depicted in column 8 of Table II B, application no. 12, and being depicted in the same respective line as said B2099, and preferably the activity is increased non-targeted, whereby the respective line discloses in column 7 the fine chemical hexadecadienoic acid.

Accordingly, in one embodiment, the molecule which activity is to be increased in the process of the invention is the gene product with an activity as a “b2099-protein”, preferably it is the molecule of section (a) or (b) of this paragraph.

In particular, it was observed that in plants, especially in Arabidopsis thaliana, increasing or generating the activity of a gene product non-targeted with the activity of a “b2099-protein”, preferably being encoded by a gene comprising the nucleic acid sequence SEQ ID NO. 115595, preferably the coding region thereof, conferred the production of or the increase in hexadecadienoic acid compared with the wild type control.

The nucleic acid sequence of B2099 from Escherichia coli, e.g. as shown in the respective line in column 5 of Table I, application no. 12, has been published in B. And the activity of the gene product thereof is the activity of b2099-protein.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “b2099-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said B2099, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B2099, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B2099, or a functional equivalent or a homolog thereof as depicted in column 8 of Table II, application no. 12, preferably a homolog or functional equivalent as depicted in column 8 of Table II B, application no. 12, and being depicted in the same respective line as said B2099, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “b2099-protein”, preferably it is the molecule of section (a) or (b) of this paragraph.

In particular, it was observed that in plants, especially in Arabidopsis thaliana, increasing or generating the activity of a gene product non-targeted with the activity of a “b2099-protein”, preferably being encoded by a gene comprising the nucleic acid sequence SEQ ID NO. 115595, preferably the coding region thereof, conferred the production of or the increase in hexadecatrienoic acid compared with the wild type control.

The nucleic acid sequence of B2107 from Escherichia coli, e.g. as shown in the respective line in column 5 of Table I, application no. 12, has been published in B. And the activity of the gene product thereof is the activity of b2107-protein.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “b2107-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said B2107, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B2107, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B2107, or a functional equivalent or a homolog thereof as depicted in column 8 of Table II, application no. 12, preferably a homolog or functional equivalent as depicted in column 8 of Table II B, application no. 12, and being depicted in the same respective line as said B2107, and preferably the activity is increased non-targeted, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “b2107-protein”, preferably it is the molecule of section (a) or (b) of this paragraph.

In particular, it was observed that in plants, especially in Arabidopsis thaliana, increasing or generating the activity of a gene product non-targeted with the activity of a “b2107-protein”, preferably being encoded by a gene comprising the nucleic acid sequence SEQ ID NO. 39002, preferably the coding region thereof, conferred the production of or the increase in 2-hydroxytetracosenoic acid (preferably 2-hydroxynervonic acid) compared with the wild type control.

The nucleic acid sequence of B2126 from Escherichia coli, e.g. as shown in the respective line in column 5 of Table I, application no. 12, has been published in B. And the activity of the gene product thereof is the activity of b2126-protein.

Accordingly, in one embodiment, the process of the present invention for producing lauric acid in a non-human organism, like a microorganism or a plant or a part thereof, comprises increasing or generating the activity of a gene product with the activity of a gene product conferring the activity of “b2126-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical lauric acid), application no. 12, preferably the coding region thereof, or a homolog or a fragment thereof, and being depicted in the same respective line as said B2126, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B2126, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B2126, or a functional equivalent or a homolog thereof as depicted in column 8 of Table II, application no. 12, preferably a homolog or functional equivalent as depicted in column 8 of Table II B, application no. 12, and being depicted in the same respective line as said B2126, and preferably the activity is increased plastidic, whereby the respective line discloses in column 7 the fine chemical lauric acid.

Accordingly, in one embodiment, the molecule which activity is to be increased in the process of the invention is the gene product with an activity as a “b2126-protein”, preferably it is the molecule of section (a) or (b) of this paragraph.

In particular, it was observed that in plants, especially in Arabidopsis thaliana, increasing or generating the activity of a gene product plastidic with the activity of a “b2126-protein”, preferably being encoded by a gene comprising the nucleic acid sequence SEQ ID NO. 124992, preferably the coding region thereof, conferred the production of or the increase in lauric acid compared with the wild type control.

The nucleic acid sequence of B2139 from Escherichia coli, e.g. as shown in the respective line in column 5 of Table I, application no. 12, has been published in B. And the activity of the gene product thereof is the activity of b2139-protein.

Accordingly, in one embodiment, the process of the present invention for producing 2-hydroxypalmitic acid in a non-human organism, like a microorganism or a plant or a part thereof, comprises increasing or generating the activity of a gene product with the activity of a gene product conferring the activity of “b2139-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxypalmitic acid), application no. 12, preferably the coding region thereof, or a homolog or a fragment thereof, and being depicted in the same respective line as said B2139, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B2139, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B2139, or a functional equivalent or a homolog thereof as depicted in column 8 of Table II, application no. 12, preferably a homolog or functional equivalent as depicted in column 8 of Table II B, application no. 12, and being depicted in the same respective line as said B2139, and preferably the activity is increased plastidic, whereby the respective line discloses in column 7 the fine chemical 2-hydroxypalmitic acid.

Accordingly, in one embodiment, the molecule which activity is to be increased in the process of the invention is the gene product with an activity as a “b2139-protein”, preferably it is the molecule of section (a) or (b) of this paragraph.

In particular, it was observed that in plants, especially in Arabidopsis thaliana, increasing or generating the activity of a gene product plastidic with the activity of a “b2139-protein”, preferably being encoded by a gene comprising the nucleic acid sequence SEQ ID NO. 125085, preferably the coding region thereof, conferred the production of or the increase in 2-hydroxypalmitic acid compared with the wild type control.

The nucleic acid sequence of B2178 from Escherichia coli, e.g. as shown in the respective line in column 5 of Table I, application no. 12, has been published in B. And the activity of the gene product thereof is the activity of ABC transporter permease protein.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “ABC transporter permease protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said B2178, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B2178, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B2178, or a functional equivalent or a homolog thereof as depicted in column 8 of Table II, application no. 12, preferably a homolog or functional equivalent as depicted in column 8 of Table II B, application no. 12, and being depicted in the same respective line as said B2178, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “ABC transporter permease protein”, preferably being encoded by a gene comprising the nucleic acid sequence SEQ ID NO. 39040, preferably the coding region thereof, conferred the production of or the increase in hexadecatrienoic acid compared with the wild type control.

The nucleic acid sequence of B2216 from Escherichia coli, e.g. as shown in the respective line in column 5 of Table I, application no. 12, has been published in B. And the activity of the gene product thereof is the activity of sensor kinase.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “sensor kinase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said B2216, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B2216, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B2216, or a functional equivalent or a homolog thereof as depicted in column 8 of Table II, application no. 12, preferably a homolog or functional equivalent as depicted in column 8 of Table II B, application no. 12, and being depicted in the same respective line as said B2216, and preferably the activity is increased plastidic, whereby the respective line discloses in column 7 the fine chemical 2-hydroxybehenic acid.

Accordingly, in one embodiment, the molecule which activity is to be increased in the process of the invention is the gene product with an activity as a “sensor kinase”, preferably it is the molecule of section (a) or (b) of this paragraph.

In particular, it was observed that in plants, especially in Arabidopsis thaliana, increasing or generating the activity of a gene product plastidic with the activity of a “sensor kinase”, preferably being encoded by a gene comprising the nucleic acid sequence SEQ ID NO. 125105, preferably the coding region thereof, conferred the production of or the increase in 2-hydroxybehenic acid compared with the wild type control.

The nucleic acid sequence of B2360 from Escherichia coli, e.g. as shown in the respective line in column 5 of Table I, application no. 12, has been published in B. And the activity of the gene product thereof is the activity of b2360-protein.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “b2360-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said B2360, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B2360, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B2360, or a functional equivalent or a homolog thereof as depicted in column 8 of Table II, application no. 12, preferably a homolog or functional equivalent as depicted in column 8 of Table II B, application no. 12, and being depicted in the same respective line as said B2360, and preferably the activity is increased non-targeted, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “b2360-protein”, preferably it is the molecule of section (a) or (b) of this paragraph.

In particular, it was observed that in plants, especially in Arabidopsis thaliana, increasing or generating the activity of a gene product non-targeted with the activity of a “b2360-protein”, preferably being encoded by a gene comprising the nucleic acid sequence SEQ ID NO. 39219, preferably the coding region thereof, conferred the production of or the increase in 2-hydroxytetracosenoic acid (preferably 2-hydroxynervonic acid) compared with the wild type control.

The nucleic acid sequence of B2414 from Escherichia coli, e.g. as shown in the respective line in column 5 of Table I, application no. 12, has been published in B. And the activity of the gene product thereof is the activity of cysteine synthase A.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “cysteine synthase A”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said B2414, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B2414, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B2414, or a functional equivalent or a homolog thereof as depicted in column 8 of Table II, application no. 12, preferably a homolog or functional equivalent as depicted in column 8 of Table II B, application no. 12, and being depicted in the same respective line as said B2414, and preferably the activity is increased non-targeted, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “cysteine synthase A”, preferably it is the molecule of section (a) or (b) of this paragraph.

In particular, it was observed that in plants, especially in Arabidopsis thaliana, increasing or generating the activity of a gene product non-targeted with the activity of a “cysteine synthase A”, preferably being encoded by a gene comprising the nucleic acid sequence SEQ ID NO. 39300, preferably the coding region thereof, conferred the production of or the increase in 2-hydroxytetracosenoic acid (preferably 2-hydroxynervonic acid) compared with the wild type control.

The nucleic acid sequence of B2439 from Escherichia coli, e.g. as shown in the respective line in column 5 of Table I, application no. 12, has been published in B. And the activity of the gene product thereof is the activity of ethanolamine utilization protein.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “ethanolamine utilization protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said B2439, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B2439, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B2439, or a functional equivalent or a homolog thereof as depicted in column 8 of Table II, application no. 12, preferably a homolog or functional equivalent as depicted in column 8 of Table II B, application no. 12, and being depicted in the same respective line as said B2439, and preferably the activity is increased non-targeted, whereby the respective line discloses in column 7 the fine chemical hexadecadienoic acid.

Accordingly, in one embodiment, the molecule which activity is to be increased in the process of the invention is the gene product with an activity as a “ethanolamine utilization protein”, preferably it is the molecule of section (a) or (b) of this paragraph.

In particular, it was observed that in plants, especially in Arabidopsis thaliana, increasing or generating the activity of a gene product non-targeted with the activity of a “ethanolamine utilization protein”, preferably being encoded by a gene comprising the nucleic acid sequence SEQ ID NO. 115628, preferably the coding region thereof, conferred the production of or the increase in hexadecadienoic acid compared with the wild type control.

The nucleic acid sequence of B2439 from Escherichia coli, e.g. as shown in the respective line in column 5 of Table I, application no. 12, has been published in B. And the activity of the gene product thereof is the activity of ethanolamine utilization protein.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “ethanolamine utilization protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said B2439, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B2439, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B2439, or a functional equivalent or a homolog thereof as depicted in column 8 of Table II, application no. 12, preferably a homolog or functional equivalent as depicted in column 8 of Table II B, application no. 12, and being depicted in the same respective line as said B2439, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “ethanolamine utilization protein”, preferably it is the molecule of section (a) or (b) of this paragraph.

In particular, it was observed that in plants, especially in Arabidopsis thaliana, increasing or generating the activity of a gene product non-targeted with the activity of a “ethanolamine utilization protein”, preferably being encoded by a gene comprising the nucleic acid sequence SEQ ID NO. 115628, preferably the coding region thereof, conferred the production of or the increase in hexadecatrienoic acid compared with the wild type control.

The nucleic acid sequence of B2439 from Escherichia coli, e.g. as shown in the respective line in column 5 of Table I, application no. 12, has been published in B. And the activity of the gene product thereof is the activity of ethanolamine utilization protein.

Accordingly, in one embodiment, the process of the present invention for producing oleic acid in a non-human organism, like a microorganism or a plant or a part thereof, comprises increasing or generating the activity of a gene product with the activity of a gene product conferring the activity of “ethanolamine utilization protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical oleic acid), application no. 12, preferably the coding region thereof, or a homolog or a fragment thereof, and being depicted in the same respective line as said B2439, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B2439, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B2439, or a functional equivalent or a homolog thereof as depicted in column 8 of Table II, application no. 12, preferably a homolog or functional equivalent as depicted in column 8 of Table II B, application no. 12, and being depicted in the same respective line as said B2439, and preferably the activity is increased non-targeted, whereby the respective line discloses in column 7 the fine chemical oleic acid.

Accordingly, in one embodiment, the molecule which activity is to be increased in the process of the invention is the gene product with an activity as a “ethanolamine utilization protein”, preferably it is the molecule of section (a) or (b) of this paragraph.

In particular, it was observed that in plants, especially in Arabidopsis thaliana, increasing or generating the activity of a gene product non-targeted with the activity of a “ethanolamine utilization protein”, preferably being encoded by a gene comprising the nucleic acid sequence SEQ ID NO. 115628, preferably the coding region thereof, conferred the production of or the increase in oleic acid compared with the wild type control.

The nucleic acid sequence of B2474 from Escherichia coli, e.g. as shown in the respective line in column 5 of Table I, application no. 12, has been published in B. And the activity of the gene product thereof is the activity of b2474-protein.

Accordingly, in one embodiment, the process of the present invention for producing cerebronic acid in a non-human organism, like a microorganism or a plant or a part thereof, comprises increasing or generating the activity of a gene product with the activity of a gene product conferring the activity of “b2474-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical cerebronic acid), application no. 12, preferably the coding region thereof, or a homolog or a fragment thereof, and being depicted in the same respective line as said B2474, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B2474, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B2474, or a functional equivalent or a homolog thereof as depicted in column 8 of Table II, application no. 12, preferably a homolog or functional equivalent as depicted in column 8 of Table II B, application no. 12, and being depicted in the same respective line as said B2474, and preferably the activity is increased non-targeted, whereby the respective line discloses in column 7 the fine chemical cerebronic acid.

Accordingly, in one embodiment, the molecule which activity is to be increased in the process of the invention is the gene product with an activity as a “b2474-protein”, preferably it is the molecule of section (a) or (b) of this paragraph.

In particular, it was observed that in plants, especially in Arabidopsis thaliana, increasing or generating the activity of a gene product non-targeted with the activity of a “b2474-protein”, preferably being encoded by a gene comprising the nucleic acid sequence SEQ ID NO. 40329, preferably the coding region thereof, conferred the production of or the increase in cerebronic acid compared with the wild type control.

The nucleic acid sequence of B2474 from Escherichia coli, e.g. as shown in the respective line in column 5 of Table I, application no. 12, has been published in B. And the activity of the gene product thereof is the activity of b2474-protein.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “b2474-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said B2474, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B2474, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B2474, or a functional equivalent or a homolog thereof as depicted in column 8 of Table II, application no. 12, preferably a homolog or functional equivalent as depicted in column 8 of Table II B, application no. 12, and being depicted in the same respective line as said B2474, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “b2474-protein”, preferably it is the molecule of section (a) or (b) of this paragraph.

In particular, it was observed that in plants, especially in Arabidopsis thaliana, increasing or generating the activity of a gene product non-targeted with the activity of a “b2474-protein”, preferably being encoded by a gene comprising the nucleic acid sequence SEQ ID NO. 40329, preferably the coding region thereof, conferred the production of or the increase in hexadecatrienoic acid compared with the wild type control.

The nucleic acid sequence of B2474 from Escherichia coli, e.g. as shown in the respective line in column 5 of Table I, application no. 12, has been published in B. And the activity of the gene product thereof is the activity of b2474-protein.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “b2474-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said B2474, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B2474, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B2474, or a functional equivalent or a homolog thereof as depicted in column 8 of Table II, application no. 12, preferably a homolog or functional equivalent as depicted in column 8 of Table II B, application no. 12, and being depicted in the same respective line as said B2474, and preferably the activity is increased non-targeted, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “b2474-protein”, preferably it is the molecule of section (a) or (b) of this paragraph.

In particular, it was observed that in plants, especially in Arabidopsis thaliana, increasing or generating the activity of a gene product non-targeted with the activity of a “b2474-protein”, preferably being encoded by a gene comprising the nucleic acid sequence SEQ ID NO. 40329, preferably the coding region thereof, conferred the production of or the increase in 2-hydroxytetracosenoic acid (preferably 2-hydroxynervonic acid) compared with the wild type control.

The nucleic acid sequence of B2513 from Escherichia coli, e.g. as shown in the respective line in column 5 of Table I, application no. 12, has been published in B. And the activity of the gene product thereof is the activity of b2513-protein.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “b2513-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said B2513, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B2513, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B2513, or a functional equivalent or a homolog thereof as depicted in column 8 of Table II, application no. 12, preferably a homolog or functional equivalent as depicted in column 8 of Table II B, application no. 12, and being depicted in the same respective line as said B2513, and preferably the activity is increased non-targeted, whereby the respective line discloses in column 7 the fine chemical hexadecadienoic acid.

Accordingly, in one embodiment, the molecule which activity is to be increased in the process of the invention is the gene product with an activity as a “b2513-protein”, preferably it is the molecule of section (a) or (b) of this paragraph.

In particular, it was observed that in plants, especially in Arabidopsis thaliana, increasing or generating the activity of a gene product non-targeted with the activity of a “b2513-protein”, preferably being encoded by a gene comprising the nucleic acid sequence SEQ ID NO. 9167, preferably the coding region thereof, conferred the production of or the increase in hexadecadienoic acid compared with the wild type control.

The nucleic acid sequence of B2585 from Escherichia coli, e.g. as shown in the respective line in column 5 of Table I, application no. 12, has been published in B. And the activity of the gene product thereof is the activity of phosphatidylserine synthase.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “phosphatidylserine synthase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said B2585, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B2585, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B2585, or a functional equivalent or a homolog thereof as depicted in column 8 of Table II, application no. 12, preferably a homolog or functional equivalent as depicted in column 8 of Table II B, application no. 12, and being depicted in the same respective line as said B2585, and preferably the activity is increased non-targeted, whereby the respective line discloses in column 7 the fine chemical hexadecadienoic acid.

Accordingly, in one embodiment, the molecule which activity is to be increased in the process of the invention is the gene product with an activity as a “phosphatidylserine synthase”, preferably it is the molecule of section (a) or (b) of this paragraph.

In particular, it was observed that in plants, especially in Arabidopsis thaliana, increasing or generating the activity of a gene product non-targeted with the activity of a “phosphatidylserine synthase”, preferably being encoded by a gene comprising the nucleic acid sequence SEQ ID NO. 125142, preferably the coding region thereof, conferred the production of or the increase in hexadecadienoic acid compared with the wild type control.

The nucleic acid sequence of B2654 from Escherichia coli, e.g. as shown in the respective line in column 5 of Table I, application no. 12, has been published in B. And the activity of the gene product thereof is the activity of b2654-protein.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “b2654-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said B2654, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B2654, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B2654, or a functional equivalent or a homolog thereof as depicted in column 8 of Table II, application no. 12, preferably a homolog or functional equivalent as depicted in column 8 of Table II B, application no. 12, and being depicted in the same respective line as said B2654, and preferably the activity is increased non-targeted, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “b2654-protein”, preferably it is the molecule of section (a) or (b) of this paragraph.

In particular, it was observed that in plants, especially in Arabidopsis thaliana, increasing or generating the activity of a gene product non-targeted with the activity of a “b2654-protein”, preferably being encoded by a gene comprising the nucleic acid sequence SEQ ID NO. 125226, preferably the coding region thereof, conferred the production of or the increase in 2-hydroxytetracosenoic acid (preferably 2-hydroxynervonic acid) compared with the wild type control.

The nucleic acid sequence of B2747 from Escherichia coli, e.g. as shown in the respective line in column 5 of Table I, application no. 12, has been published in B. And the activity of the gene product thereof is the activity of 2-C-methyl-D-erythritol 4-phosphate cytidylyltransferase.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “2-C-methyl-D-erythritol 4-phosphate cytidylyltransferase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said B2747, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B2747, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B2747, or a functional equivalent or a homolog thereof as depicted in column 8 of Table II, application no. 12, preferably a homolog or functional equivalent as depicted in column 8 of Table II B, application no. 12, and being depicted in the same respective line as said B2747, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “2-C-methyl-D-erythritol 4-phosphate cytidylyltransferase”, preferably it is the molecule of section (a) or (b) of this paragraph.

In particular, it was observed that in plants, especially in Arabidopsis thaliana, increasing or generating the activity of a gene product non-targeted with the activity of a “2-C-methyl-D-erythritol 4-phosphate cytidylyltransferase”, preferably being encoded by a gene comprising the nucleic acid sequence SEQ ID NO. 81075, preferably the coding region thereof, conferred the production of or the increase in hexadecatrienoic acid compared with the wild type control.

The nucleic acid sequence of B2753 from Escherichia coli, e.g. as shown in the respective line in column 5 of Table I, application no. 12, has been published in B. And the activity of the gene product thereof is the activity of alkaline phosphatase isozyme conversion protein.

Accordingly, in one embodiment, the process of the present invention for producing eicosanoic acid in a non-human organism, like a microorganism or a plant or a part thereof, comprises increasing or generating the activity of a gene product with the activity of a gene product conferring the activity of “alkaline phosphatase isozyme conversion protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical eicosanoic acid), application no. 12, preferably the coding region thereof, or a homolog or a fragment thereof, and being depicted in the same respective line as said B2753, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B2753, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B2753, or a functional equivalent or a homolog thereof as depicted in column 8 of Table II, application no. 12, preferably a homolog or functional equivalent as depicted in column 8 of Table II B, application no. 12, and being depicted in the same respective line as said B2753, and preferably the activity is increased non-targeted, whereby the respective line discloses in column 7 the fine chemical eicosanoic acid.

Accordingly, in one embodiment, the molecule which activity is to be increased in the process of the invention is the gene product with an activity as a “alkaline phosphatase isozyme conversion protein”, preferably it is the molecule of section (a) or (b) of this paragraph.

In particular, it was observed that in plants, especially in Arabidopsis thaliana, increasing or generating the activity of a gene product non-targeted with the activity of a “alkaline phosphatase isozyme conversion protein”, preferably being encoded by a gene comprising the nucleic acid sequence SEQ ID NO. 125234, preferably the coding region thereof, conferred the production of or the increase in eicosanoic acid compared with the wild type control.

The nucleic acid sequence of B2963 from Escherichia coli, e.g. as shown in the respective line in column 5 of Table I, application no. 12, has been published in B. And the activity of the gene product thereof is the activity of murein transglycosylase.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “murein transglycosylase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said B2963, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B2963, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B2963, or a functional equivalent or a homolog thereof as depicted in column 8 of Table II, application no. 12, preferably a homolog or functional equivalent as depicted in column 8 of Table II B, application no. 12, and being depicted in the same respective line as said B2963, and preferably the activity is increased plastidic, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “murein transglycosylase”, preferably it is the molecule of section (a) or (b) of this paragraph.

In particular, it was observed that in plants, especially in Arabidopsis thaliana, increasing or generating the activity of a gene product plastidic with the activity of a “murein transglycosylase”, preferably being encoded by a gene comprising the nucleic acid sequence SEQ ID NO. 41732, preferably the coding region thereof, conferred the production of or the increase in 2-hydroxytetracosenoic acid (preferably 2-hydroxynervonic acid) compared with the wild type control.

The nucleic acid sequence of B3442 from Escherichia coli, e.g. as shown in the respective line in column 5 of Table I, application no. 12, has been published in B. And the activity of the gene product thereof is the activity of b3442-protein.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “b3442-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said B3442, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B3442, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B3442, or a functional equivalent or a homolog thereof as depicted in column 8 of Table II, application no. 12, preferably a homolog or functional equivalent as depicted in column 8 of Table II B, application no. 12, and being depicted in the same respective line as said B3442, and preferably the activity is increased non-targeted, whereby the respective line discloses in column 7 the fine chemical hexadecadienoic acid.

Accordingly, in one embodiment, the molecule which activity is to be increased in the process of the invention is the gene product with an activity as a “b3442-protein”, preferably it is the molecule of section (a) or (b) of this paragraph.

In particular, it was observed that in plants, especially in Arabidopsis thaliana, increasing or generating the activity of a gene product non-targeted with the activity of a “b3442-protein”, preferably being encoded by a gene comprising the nucleic acid sequence SEQ ID NO. 94380, preferably the coding region thereof, conferred the production of or the increase in hexadecadienoic acid compared with the wild type control.

The nucleic acid sequence of B3568 from Escherichia coli, e.g. as shown in the respective line in column 5 of Table I, application no. 12, has been published in B. And the activity of the gene product thereof is the activity of sugar transport system permease protein.

Accordingly, in one embodiment, the process of the present invention for producing cerebronic acid in a non-human organism, like a microorganism or a plant or a part thereof, comprises increasing or generating the activity of a gene product with the activity of a gene product conferring the activity of “sugar transport system permease protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical cerebronic acid), application no. 12, preferably the coding region thereof, or a homolog or a fragment thereof, and being depicted in the same respective line as said B3568, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B3568, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B3568, or a functional equivalent or a homolog thereof as depicted in column 8 of Table II, application no. 12, preferably a homolog or functional equivalent as depicted in column 8 of Table II B, application no. 12, and being depicted in the same respective line as said B3568, and preferably the activity is increased plastidic, whereby the respective line discloses in column 7 the fine chemical cerebronic acid.

Accordingly, in one embodiment, the molecule which activity is to be increased in the process of the invention is the gene product with an activity as a “sugar transport system permease protein”, preferably it is the molecule of section (a) or (b) of this paragraph.

In particular, it was observed that in plants, especially in Arabidopsis thaliana, increasing or generating the activity of a gene product plastidic with the activity of a “sugar transport system permease protein”, preferably being encoded by a gene comprising the nucleic acid sequence SEQ ID NO. 125264, preferably the coding region thereof, conferred the production of or the increase in cerebronic acid compared with the wild type control.

The nucleic acid sequence of B3812 from Escherichia coli, e.g. as shown in the respective line in column 5 of Table I, application no. 12, has been published in B. And the activity of the gene product thereof is the activity of b3812-protein.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “b3812-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said B3812, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B3812, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B3812, or a functional equivalent or a homolog thereof as depicted in column 8 of Table II, application no. 12, preferably a homolog or functional equivalent as depicted in column 8 of Table II B, application no. 12, and being depicted in the same respective line as said B3812, and preferably the activity is increased non-targeted, whereby the respective line discloses in column 7 the fine chemical 2-hydroxybehenic acid.

Accordingly, in one embodiment, the molecule which activity is to be increased in the process of the invention is the gene product with an activity as a “b3812-protein”, preferably it is the molecule of section (a) or (b) of this paragraph.

In particular, it was observed that in plants, especially in Arabidopsis thaliana, increasing or generating the activity of a gene product non-targeted with the activity of a “b3812-protein”, preferably being encoded by a gene comprising the nucleic acid sequence SEQ ID NO. 125487, preferably the coding region thereof, conferred the production of or the increase in 2-hydroxybehenic acid compared with the wild type control.

The nucleic acid sequence of B3845 from Escherichia coli, e.g. as shown in the respective line in column 5 of Table I, application no. 12, has been published in B. And the activity of the gene product thereof is the activity of 3-ketoacyl-CoA thiolase.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “3-ketoacyl-CoA thiolase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said B3845, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B3845, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B3845, or a functional equivalent or a homolog thereof as depicted in column 8 of Table II, application no. 12, preferably a homolog or functional equivalent as depicted in column 8 of Table II B, application no. 12, and being depicted in the same respective line as said B3845, and preferably the activity is increased non-targeted, whereby the respective line discloses in column 7 the fine chemical hexadecadienoic acid.

Accordingly, in one embodiment, the molecule which activity is to be increased in the process of the invention is the gene product with an activity as a “3-ketoacyl-CoA thiolase”, preferably it is the molecule of section (a) or (b) of this paragraph.

In particular, it was observed that in plants, especially in Arabidopsis thaliana, increasing or generating the activity of a gene product non-targeted with the activity of a “3-ketoacyl-CoA thiolase”, preferably being encoded by a gene comprising the nucleic acid sequence SEQ ID NO. 100675, preferably the coding region thereof, conferred the production of or the increase in hexadecadienoic acid compared with the wild type control.

The nucleic acid sequence of B3845 from Escherichia coli, e.g. as shown in the respective line in column 5 of Table I, application no. 12, has been published in B. And the activity of the gene product thereof is the activity of 3-ketoacyl-CoA thiolase.

Accordingly, in one embodiment, the process of the present invention for producing oleic acid in a non-human organism, like a microorganism or a plant or a part thereof, comprises increasing or generating the activity of a gene product with the activity of a gene product conferring the activity of “3-ketoacyl-CoA thiolase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical oleic acid), application no. 12, preferably the coding region thereof, or a homolog or a fragment thereof, and being depicted in the same respective line as said B3845, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B3845, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B3845, or a functional equivalent or a homolog thereof as depicted in column 8 of Table II, application no. 12, preferably a homolog or functional equivalent as depicted in column 8 of Table II B, application no. 12, and being depicted in the same respective line as said B3845, and preferably the activity is increased plastidic, whereby the respective line discloses in column 7 the fine chemical oleic acid.

Accordingly, in one embodiment, the molecule which activity is to be increased in the process of the invention is the gene product with an activity as a “3-ketoacyl-CoA thiolase”, preferably it is the molecule of section (a) or (b) of this paragraph.

In particular, it was observed that in plants, especially in Arabidopsis thaliana, increasing or generating the activity of a gene product plastidic with the activity of a “3-ketoacyl-CoA thiolase”, preferably being encoded by a gene comprising the nucleic acid sequence SEQ ID NO. 100675, preferably the coding region thereof, conferred the production of or the increase in oleic acid compared with the wild type control.

The nucleic acid sequence of B3872 from Escherichia coli, e.g. as shown in the respective line in column 5 of Table I, application no. 12, has been published in B. And the activity of the gene product thereof is the activity of transcriptional regulator.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said B3872, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B3872, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B3872, or a functional equivalent or a homolog thereof as depicted in column 8 of Table II, application no. 12, preferably a homolog or functional equivalent as depicted in column 8 of Table II B, application no. 12, and being depicted in the same respective line as said B3872, and preferably the activity is increased plastidic, whereby the respective line discloses in column 7 the fine chemical hexadecadienoic acid.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product plastidic with the activity of a “transcriptional regulator”, preferably being encoded by a gene comprising the nucleic acid sequence SEQ ID NO. 83821, preferably the coding region thereof, conferred the production of or the increase in hexadecadienoic acid compared with the wild type control.

The nucleic acid sequence of B3925 from Escherichia coli, e.g. as shown in the respective line in column 5 of Table I, application no. 12, has been published in B. And the activity of the gene product thereof is the activity of fructose 1,6-bisphosphatase.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “fructose 1,6-bisphosphatase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said B3925, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B3925, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B3925, or a functional equivalent or a homolog thereof as depicted in column 8 of Table II, application no. 12, preferably a homolog or functional equivalent as depicted in column 8 of Table II B, application no. 12, and being depicted in the same respective line as said B3925, and preferably the activity is increased non-targeted, whereby the respective line discloses in column 7 the fine chemical hexadecadienoic acid.

Accordingly, in one embodiment, the molecule which activity is to be increased in the process of the invention is the gene product with an activity as a “fructose 1,6-bisphosphatase”, preferably it is the molecule of section (a) or (b) of this paragraph.

In particular, it was observed that in plants, especially in Arabidopsis thaliana, increasing or generating the activity of a gene product non-targeted with the activity of a “fructose 1,6-bisphosphatase”, preferably being encoded by a gene comprising the nucleic acid sequence SEQ ID NO. 125541, preferably the coding region thereof, conferred the production of or the increase in hexadecadienoic acid compared with the wild type control.

The nucleic acid sequence of B3939 from Escherichia coli, e.g. as shown in the respective line in column 5 of Table I, application no. 12, has been published in B. And the activity of the gene product thereof is the activity of cystathionine gamma-synthase.

Accordingly, in one embodiment, the process of the present invention for producing behenic acid in a non-human organism, like a microorganism or a plant or a part thereof, comprises increasing or generating the activity of a gene product with the activity of a gene product conferring the activity of “cystathionine gamma-synthase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical behenic acid), application no. 12, preferably the coding region thereof, or a homolog or a fragment thereof, and being depicted in the same respective line as said B3939, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B3939, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B3939, or a functional equivalent or a homolog thereof as depicted in column 8 of Table II, application no. 12, preferably a homolog or functional equivalent as depicted in column 8 of Table II B, application no. 12, and being depicted in the same respective line as said B3939, and preferably the activity is increased plastidic, whereby the respective line discloses in column 7 the fine chemical behenic acid.

Accordingly, in one embodiment, the molecule which activity is to be increased in the process of the invention is the gene product with an activity as a “cystathionine gamma-synthase”, preferably it is the molecule of section (a) or (b) of this paragraph.

In particular, it was observed that in plants, especially in Arabidopsis thaliana, increasing or generating the activity of a gene product plastidic with the activity of a “cystathionine gamma-synthase”, preferably being encoded by a gene comprising the nucleic acid sequence SEQ ID NO. 125795, preferably the coding region thereof, conferred the production of or the increase in behenic acid compared with the wild type control.

The nucleic acid sequence of B3939 from Escherichia coli, e.g. as shown in the respective line in column 5 of Table I, application no. 12, has been published in B. And the activity of the gene product thereof is the activity of cystathionine gamma-synthase.

Accordingly, in one embodiment, the process of the present invention for producing heptadecanoic acid in a non-human organism, like a microorganism or a plant or a part thereof, comprises increasing or generating the activity of a gene product with the activity of a gene product conferring the activity of “cystathionine gamma-synthase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical heptadecanoic acid), application no. 12, preferably the coding region thereof, or a homolog or a fragment thereof, and being depicted in the same respective line as said B3939, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B3939, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B3939, or a functional equivalent or a homolog thereof as depicted in column 8 of Table II, application no. 12, preferably a homolog or functional equivalent as depicted in column 8 of Table II B, application no. 12, and being depicted in the same respective line as said B3939, and preferably the activity is increased plastidic, whereby the respective line discloses in column 7 the fine chemical heptadecanoic acid.

Accordingly, in one embodiment, the molecule which activity is to be increased in the process of the invention is the gene product with an activity as a “cystathionine gamma-synthase”, preferably it is the molecule of section (a) or (b) of this paragraph.

In particular, it was observed that in plants, especially in Arabidopsis thaliana, increasing or generating the activity of a gene product plastidic with the activity of a “cystathionine gammasynthase”, preferably being encoded by a gene comprising the nucleic acid sequence SEQ ID NO. 125795, preferably the coding region thereof, conferred the production of or the increase in heptadecanoic acid compared with the wild type control.

The nucleic acid sequence of B3939 from Escherichia coli, e.g. as shown in the respective line in column 5 of Table I, application no. 12, has been published in B. And the activity of the gene product thereof is the activity of cystathionine gamma-synthase.

Accordingly, in one embodiment, the process of the present invention for producing oleic acid in a non-human organism, like a microorganism or a plant or a part thereof, comprises increasing or generating the activity of a gene product with the activity of a gene product conferring the activity of “cystathionine gamma-synthase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical oleic acid), application no. 12, preferably the coding region thereof, or a homolog or a fragment thereof, and being depicted in the same respective line as said B3939, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B3939, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B3939, or a functional equivalent or a homolog thereof as depicted in column 8 of Table II, application no. 12, preferably a homolog or functional equivalent as depicted in column 8 of Table II B, application no. 12, and being depicted in the same respective line as said B3939, and preferably the activity is increased plastidic, whereby the respective line discloses in column 7 the fine chemical oleic acid.

Accordingly, in one embodiment, the molecule which activity is to be increased in the process of the invention is the gene product with an activity as a “cystathionine gamma-synthase”, preferably it is the molecule of section (a) or (b) of this paragraph.

In particular, it was observed that in plants, especially in Arabidopsis thaliana, increasing or generating the activity of a gene product plastidic with the activity of a “cystathionine gamma-synthase”, preferably being encoded by a gene comprising the nucleic acid sequence SEQ ID NO. 125795, preferably the coding region thereof, conferred the production of or the increase in oleic acid compared with the wild type control.

The nucleic acid sequence of B3944 from Escherichia coli, e.g. as shown in the respective line in column 5 of Table I, application no. 12, has been published in B. And the activity of the gene product thereof is the activity of b3944-protein.

Accordingly, in one embodiment, the process of the present invention for producing 2-hydroxypalmitic acid in a non-human organism, like a microorganism or a plant or a part thereof, comprises increasing or generating the activity of a gene product with the activity of a gene product conferring the activity of “b3944-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxypalmitic acid), application no. 12, preferably the coding region thereof, or a homolog or a fragment thereof, and being depicted in the same respective line as said B3944, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B3944, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B3944, or a functional equivalent or a homolog thereof as depicted in column 8 of Table II, application no. 12, preferably a homolog or functional equivalent as depicted in column 8 of Table II B, application no. 12, and being depicted in the same respective line as said B3944, and preferably the activity is increased plastidic, whereby the respective line discloses in column 7 the fine chemical 2-hydroxypalmitic acid.

Accordingly, in one embodiment, the molecule which activity is to be increased in the process of the invention is the gene product with an activity as a “b3944-protein”, preferably it is the molecule of section (a) or (b) of this paragraph.

In particular, it was observed that in plants, especially in Arabidopsis thaliana, increasing or generating the activity of a gene product plastidic with the activity of a “b3944-protein”, preferably being encoded by a gene comprising the nucleic acid sequence SEQ ID NO. 126377, preferably the coding region thereof, conferred the production of or the increase in 2-hydroxypalmitic acid compared with the wild type control.

The nucleic acid sequence of B4017 from Escherichia coli, e.g. as shown in the respective line in column 5 of Table I, application no. 12, has been published in B. And the activity of the gene product thereof is the activity of ankyrin repeat protein.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “ankyrin repeat protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said B4017, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B4017, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B4017, or a functional equivalent or a homolog thereof as depicted in column 8 of Table II, application no. 12, preferably a homolog or functional equivalent as depicted in column 8 of Table II B, application no. 12, and being depicted in the same respective line as said B4017, and preferably the activity is increased plastidic, whereby the respective line discloses in column 7 the fine chemical 2-hydroxybehenic acid.

Accordingly, in one embodiment, the molecule which activity is to be increased in the process of the invention is the gene product with an activity as a “ankyrin repeat protein”, preferably it is the molecule of section (a) or (b) of this paragraph.

In particular, it was observed that in plants, especially in Arabidopsis thaliana, increasing or generating the activity of a gene product plastidic with the activity of a “ankyrin repeat protein”, preferably being encoded by a gene comprising the nucleic acid sequence SEQ ID NO. 126415, preferably the coding region thereof, conferred the production of or the increase in 2-hydroxybehenic acid compared with the wild type control.

The nucleic acid sequence of B4023 from Escherichia coli, e.g. as shown in the respective line in column 5 of Table I, application no. 12, has been published in B. And the activity of the gene product thereof is the activity of b4023-protein.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “b4023-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said B4023, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B4023, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B4023, or a functional equivalent or a homolog thereof as depicted in column 8 of Table II, application no. 12, preferably a homolog or functional equivalent as depicted in column 8 of Table II B, application no. 12, and being depicted in the same respective line as said B4023, and preferably the activity is increased non-targeted, whereby the respective line discloses in column 7 the fine chemical hexadecadienoic acid.

Accordingly, in one embodiment, the molecule which activity is to be increased in the process of the invention is the gene product with an activity as a “b4023-protein”, preferably it is the molecule of section (a) or (b) of this paragraph.

In particular, it was observed that in plants, especially in Arabidopsis thaliana, increasing or generating the activity of a gene product non-targeted with the activity of a “b4023-protein”, preferably being encoded by a gene comprising the nucleic acid sequence SEQ ID NO. 119209, preferably the coding region thereof, conferred the production of or the increase in hexadecadienoic acid compared with the wild type control.

The nucleic acid sequence of B4069 from Escherichia coli, e.g. as shown in the respective line in column 5 of Table I, application no. 12, has been published in B. And the activity of the gene product thereof is the activity of acetyl-coenzyme A synthetase.

Accordingly, in one embodiment, the process of the present invention for producing hepta-decanoic acid in a non-human organism, like a microorganism or a plant or a part thereof, com-prises increasing or generating the activity of a gene product with the activity of a gene product conferring the activity of “acetyl-coenzyme A synthetase”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical heptadecanoic acid), application no. 12, preferably the coding region thereof, or a homolog or a fragment thereof, and being depicted in the same respective line as said B4069, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B4069, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B4069, or a functional equivalent or a homolog thereof as depicted in column 8 of Table II, application no. 12, preferably a homolog or functional equivalent as depicted in column 8 of Table II B, application no. 12, and being depicted in the same respective line as said B4069, and preferably the activity is increased plastidic, whereby the respective line discloses in column 7 the fine chemical heptadecanoic acid.

Accordingly, in one embodiment, the molecule which activity is to be increased in the process of the invention is the gene product with an activity as a “acetyl-coenzyme A synthetase”, preferably it is the molecule of section (a) or (b) of this paragraph.

In particular, it was observed that in plants, especially in Arabidopsis thaliana, increasing or generating the activity of a gene product plastidic with the activity of a “acetyl-coenzyme A synthetase”, preferably being encoded by a gene comprising the nucleic acid sequence SEQ ID NO. 126421, preferably the coding region thereof, conferred the production of or the increase in heptadecanoic acid compared with the wild type control.

The nucleic acid sequence of B4205 from Escherichia coli, e.g. as shown in the respective line in column 5 of Table I, application no. 12, has been published in B. And the activity of the gene product thereof is the activity of b4205-protein.

Accordingly, in one embodiment, the process of the present invention for producing 2-hydroxypalmitic acid in a non-human organism, like a microorganism or a plant or a part thereof, comprises increasing or generating the activity of a gene product with the activity of a gene product conferring the activity of “b4205-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxypalmitic acid), application no. 12, preferably the coding region thereof, or a homolog or a fragment thereof, and being depicted in the same respective line as said B4205, or a functional equivalent or a homolog thereof as shown in column 8 of Ta-ble I, application no. 12, preferably the coding region thereof, particularly a homolog or functional equivalent as shown in column 8 of Table I B, application no. 12, and being depicted in the same respective line as said B4205, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B4205, or a functional equivalent or a homolog thereof as depicted in column 8 of Table II, application no. 12, preferably a homolog or functional equivalent as depicted in column 8 of Table II B, application no. 12, and being depicted in the same respective line as said B4205, and preferably the activity is increased plastidic, whereby the respective line discloses in column 7 the fine chemical 2-hydroxypalmitic acid.

Accordingly, in one embodiment, the molecule which activity is to be increased in the process of the invention is the gene product with an activity as a “b4205-protein”, preferably it is the molecule of section (a) or (b) of this paragraph.

In particular, it was observed that in plants, especially in Arabidopsis thaliana, increasing or generating the activity of a gene product plastidic with the activity of a “b4205-protein”, preferably being encoded by a gene comprising the nucleic acid sequence SEQ ID NO. 126975, preferably the coding region thereof, conferred the production of or the increase in 2-hydroxypalmitic acid compared with the wild type control.

The nucleic acid sequence of C_pp032007025r from Physcomitrella patens, e.g. as shown in the respective line in column 5 of Table I, application no. 12, is unpublished. And the activity of the gene product thereof is the activity of vacuolar processing enzyme.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “vacuolar processing enzyme”, especially from Physcomitrella patens or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said C_pp032007025r, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said C_pp032007025r, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said C_pp032007025r, or a functional equivalent or a homolog thereof as depicted in column 8 of Table II, application no. 12, preferably a homolog or functional equivalent as depicted in column 8 of Table II B, application no. 12, and being depicted in the same respective line as said C_pp032007025r, and preferably the activity is increased non-targeted, whereby the respective line discloses in column 7 the fine chemical 2-hydroxybehenic acid.

Accordingly, in one embodiment, the molecule which activity is to be increased in the process of the invention is the gene product with an activity as a “vacuolar processing enzyme”, preferably it is the molecule of section (a) or (b) of this paragraph.

In particular, it was observed that in plants, especially in Arabidopsis thaliana, increasing or generating the activity of a gene product non-targeted with the activity of a “vacuolar processing enzyme”, preferably being encoded by a gene comprising the nucleic acid sequence SEQ ID NO. 126991, preferably the coding region thereof, conferred the production of or the increase in 2-hydroxybehenic acid compared with the wild type control.

The nucleic acid sequence of GM02LC12622 from Glycine max, e.g. as shown in the respective line in column 5 of Table I, application no. 12, is unpublished. And the activity of the gene product thereof is the activity of glutaredoxin.

Accordingly, in one embodiment, the process of the present invention for producing 2-hydroxypalmitic acid in a non-human organism, like a microorganism or a plant or a part thereof, comprises increasing or generating the activity of a gene product with the activity of a gene product conferring the activity of “glutaredoxin”, especially from Glycine max or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxypalmitic acid), application no. 12, preferably the coding region thereof, or a homolog or a fragment thereof, and being depicted in the same respective line as said GM02LC12622, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said GM02LC12622, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said GM02LC12622, or a functional equivalent or a homolog thereof as depicted in column 8 of Table II, application no. 12, preferably a homolog or functional equivalent as depicted in column 8 of Table II B, application no. 12, and being depicted in the same respective line as said GM02LC12622, and preferably the activity is increased non-targeted, whereby the respective line discloses in column 7 the fine chemical 2-hydroxypalmitic acid.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 10811, preferably the coding region thereof, conferred the production of or the increase in 2-hydroxypalmitic acid compared with the wild type control.

The nucleic acid sequence of GM02LC19289 from Glycine max, e.g. as shown in the respective line in column 5 of Table I, application no. 12, is unpublished. And the activity of the gene product thereof is the activity of transcription factor.

Accordingly, in one embodiment, the process of the present invention for producing cerebronic acid in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I (whereby the respective line discloses in column 7 the fine chemical cerebronic acid), application no. 12, preferably the coding region thereof, or a homolog or a fragment thereof, and being depicted in the same respective line as said GM02LC19289, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said GM02LC19289, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said GM02LC19289, or a functional equivalent or a homolog thereof as depicted in column 8 of Table II, application no. 12, preferably a homolog or functional equivalent as depicted in column 8 of Table II B, application no. 12, and being depicted in the same respective line as said GM02LC19289, and preferably the activity is increased non-targeted, whereby the respective line discloses in column 7 the fine chemical cerebronic acid.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 46751, preferably the coding region thereof, conferred the production of or the increase in cerebronic acid compared with the wild type control.

The nucleic acid sequence of GM02LC19289 from Glycine max, e.g. as shown in the respective line in column 5 of Table I, application no. 12, is unpublished. And the activity of the gene product thereof is the activity of transcription factor.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said GM02LC19289, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said GM02LC19289, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said GM02LC19289, or a functional equivalent or a homolog thereof as depicted in column 8 of Table II, application no. 12, preferably a homolog or functional equivalent as depicted in column 8 of Table II B, application no. 12, and being depicted in the same respective line as said GM02LC19289, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 46751, preferably the coding region thereof, conferred the production of or the increase in hexadecatrienoic acid compared with the wild type control.

The nucleic acid sequence of GM02LC38418 from Glycine max, e.g. as shown in the respective line in column 5 of Table I, application no. 12, is unpublished. And the activity of the gene product thereof is the activity of GM02LC38418-protein.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “GM02LC38418-protein”, especially from Glycine max or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said GM02LC38418, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said GM02LC38418, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said GM02LC38418, or a functional equivalent or a homolog thereof as depicted in column 8 of Table II, application no. 12, preferably a homolog or functional equivalent as depicted in column 8 of Table II B, application no. 12, and being depicted in the same respective line as said GM02LC38418, and preferably the activity is increased non-targeted, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “GM02LC38418-protein”, preferably it is the molecule of section (a) or (b) of this paragraph.

In particular, it was observed that in plants, especially in Arabidopsis thaliana, increasing or generating the activity of a gene product non-targeted with the activity of a “GM02LC38418-protein”, preferably being encoded by a gene comprising the nucleic acid sequence SEQ ID NO. 116312, preferably the coding region thereof, conferred the production of or the increase in 2-hydroxytetracosenoic acid (preferably 2-hydroxynervonic acid) compared with the wild type control.

The nucleic acid sequence of Sll0281 from Synechocystis sp., e.g. as shown in the respective line in column 5 of Table I, application no. 12, has been published in C. And the activity of the gene product thereof is the activity of sll0281-protein.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “sll0281-protein”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said Sll0281, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Sll0281, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Sll0281, or a functional equivalent or a homolog thereof as depicted in column 8 of Table II, application no. 12, preferably a homolog or functional equivalent as depicted in column 8 of Table II B, application no. 12, and being depicted in the same respective line as said Sll0281, and preferably the activity is increased plastidic, whereby the respective line discloses in column 7 the fine chemical hexadecadienoic acid.

Accordingly, in one embodiment, the molecule which activity is to be increased in the process of the invention is the gene product with an activity as a “sll0281-protein”, preferably it is the molecule of section (a) or (b) of this paragraph.

In particular, it was observed that in plants, especially in Arabidopsis thaliana, increasing or generating the activity of a gene product plastidic with the activity of a “sll0281-protein”, preferably being encoded by a gene comprising the nucleic acid sequence SEQ ID NO. 84865, preferably the coding region thereof, conferred the production of or the increase in hexadecadienoic acid compared with the wild type control.

The nucleic acid sequence of Sll0368 from Synechocystis sp., e.g. as shown in the respective line in column 5 of Table I, application no. 12, has been published in C. And the activity of the gene product thereof is the activity of bifunctional protein (phosphoribosyltransferase and regulatory protein).

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “bifunctional protein (phosphoribosyltransferase and regulatory protein)”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said Sll0368, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Sll0368, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Sll0368, or a functional equivalent or a homolog thereof as depicted in column 8 of Table II, application no. 12, preferably a homolog or functional equivalent as depicted in column 8 of Table II B, application no. 12, and being depicted in the same respective line as said Sll0368, and preferably the activity is increased mitochondrial, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “bifunctional protein (phosphoribosyltransferase and regulatory protein)”, preferably it is the molecule of section (a) or (b) of this paragraph.

In particular, it was observed that in plants, especially in Arabidopsis thaliana, increasing or generating the activity of a gene product mitochondrial with the activity of a “bifunctional protein (phosphoribosyltransferase and regulatory protein)”, preferably being encoded by a gene comprising the nucleic acid sequence SEQ ID NO. 106920, preferably the coding region thereof, conferred the production of or the increase in 2-hydroxytetracosenoic acid (preferably 2-hydroxynervonic acid) compared with the wild type control.

The nucleic acid sequence of Sll0406 from Synechocystis sp., e.g. as shown in the respective line in column 5 of Table I, application no. 12, has been published in C. And the activity of the gene product thereof is the activity of sll0406-protein.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “sll0406-protein”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said Sll0406, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Sll0406, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Sll0406, or a functional equivalent or a homolog thereof as depicted in column 8 of Table II, application no. 12, preferably a homolog or functional equivalent as depicted in column 8 of Table II B, application no. 12, and being depicted in the same respective line as said Sll0406, and preferably the activity is increased non-targeted, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “sll0406-protein”, preferably it is the molecule of section (a) or (b) of this paragraph.

In particular, it was observed that in plants, especially in Arabidopsis thaliana, increasing or generating the activity of a gene product non-targeted with the activity of a “sll0406-protein”, preferably being encoded by a gene comprising the nucleic acid sequence SEQ ID NO. 127103, preferably the coding region thereof, conferred the production of or the increase in 2-hydroxytetracosenoic acid (preferably 2-hydroxynervonic acid) compared with the wild type control.

The nucleic acid sequence of Sll0833 from Synechocystis sp., e.g. as shown in the respective line in column 5 of Table I, application no. 12, has been published in C. And the activity of the gene product thereof is the activity of binding-protein-dependent transport systems inner membrane component.

Accordingly, in one embodiment, the process of the present invention for producing cerebronic acid in a non-human organism, like a microorganism or a plant or a part thereof, comprises increasing or generating the activity of a gene product with the activity of a gene product conferring the activity of “binding-protein-dependent transport systems inner membrane component”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical cerebronic acid), application no. 12, preferably the coding region thereof, or a homolog or a fragment thereof, and being depicted in the same respective line as said Sll0833, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Sll0833, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Sll0833, or a functional equivalent or a homolog thereof as depicted in column 8 of Table II, application no. 12, preferably a homolog or functional equivalent as depicted in column 8 of Table II B, application no. 12, and being depicted in the same respective line as said Sll0833, and preferably the activity is increased non-targeted, whereby the respective line discloses in column 7 the fine chemical cerebronic acid.

Accordingly, in one embodiment, the molecule which activity is to be increased in the process of the invention is the gene product with an activity as a “binding-protein-dependent transport systems inner membrane component”, preferably it is the molecule of section (a) or (b) of this paragraph.

In particular, it was observed that in plants, especially in Arabidopsis thaliana, increasing or generating the activity of a gene product non-targeted with the activity of a “binding-protein-dependent transport systems inner membrane component”, preferably being encoded by a gene comprising the nucleic acid sequence SEQ ID NO. 101707, preferably the coding region thereof, conferred the production of or the increase in cerebronic acid compared with the wild type control.

The nucleic acid sequence of Sll0895 from Synechocystis sp., e.g. as shown in the respective line in column 5 of Table I, application no. 12, has been published in C. And the activity of the gene product thereof is the activity of phosphatase.

Accordingly, in one embodiment, the process of the present invention for producing cerebronic acid in a non-human organism, like a microorganism or a plant or a part thereof, comprises increasing or generating the activity of a gene product with the activity of a gene product conferring the activity of “phosphatase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical cerebronic acid), application no. 12, preferably the coding region thereof, or a homolog or a fragment thereof, and being depicted in the same respective line as said Sll0895, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Sll0895, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Sll0895, or a functional equivalent or a homolog thereof as depicted in column 8 of Table II, application no. 12, preferably a homolog or functional equivalent as depicted in column 8 of Table II B, application no. 12, and being depicted in the same respective line as said Sll0895, and preferably the activity is increased non-targeted, whereby the respective line discloses in column 7 the fine chemical cerebronic acid.

Accordingly, in one embodiment, the molecule which activity is to be increased in the process of the invention is the gene product with an activity as a “phosphatase”, preferably it is the molecule of section (a) or (b) of this paragraph.

In particular, it was observed that in plants, especially in Arabidopsis thaliana, increasing or generating the activity of a gene product non-targeted with the activity of a “phosphatase”, preferably being encoded by a gene comprising the nucleic acid sequence SEQ ID NO. 127109, preferably the coding region thereof, conferred the production of or the increase in cerebronic acid compared with the wild type control.

The nucleic acid sequence of Sll0895 from Synechocystis sp., e.g. as shown in the respective line in column 5 of Table I, application no. 12, has been published in C. And the activity of the gene product thereof is the activity of phosphatase.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “phosphatase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said Sll0895, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Sll0895, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Sll0895, or a functional equivalent or a homolog thereof as depicted in column 8 of Table II, application no. 12, preferably a homolog or functional equivalent as depicted in column 8 of Table II B, application no. 12, and being depicted in the same respective line as said Sll0895, and preferably the activity is increased non-targeted, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “phosphatase”, preferably it is the molecule of section (a) or (b) of this paragraph.

In particular, it was observed that in plants, especially in Arabidopsis thaliana, increasing or generating the activity of a gene product non-targeted with the activity of a “phosphatase”, preferably being encoded by a gene comprising the nucleic acid sequence SEQ ID NO. 127109, preferably the coding region thereof, conferred the production of or the increase in 2-hydroxytetracosenoic acid (preferably 2-hydroxynervonic acid) compared with the wild type control.

The nucleic acid sequence of Sll1031 from Synechocystis sp., e.g. as shown in the respective line in column 5 of Table I, application no. 12, has been published in C. And the activity of the gene product thereof is the activity of carbon dioxide concentrating mechanism protein.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “carbon dioxide concentrating mechanism protein”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said Sll1031, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Sll1031, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Sll1031, or a functional equivalent or a homolog thereof as depicted in column 8 of Table II, application no. 12, preferably a homolog or functional equivalent as depicted in column 8 of Table II B, application no. 12, and being depicted in the same respective line as said Sll1031, and preferably the activity is increased mitochondrial, whereby the respective line discloses in column 7 the fine chemical hexadecadienoic acid.

Accordingly, in one embodiment, the molecule which activity is to be increased in the process of the invention is the gene product with an activity as a “carbon dioxide concentrating mechanism protein”, preferably it is the molecule of section (a) or (b) of this paragraph.

In particular, it was observed that in plants, especially in Arabidopsis thaliana, increasing or generating the activity of a gene product mitochondrial with the activity of a “carbon dioxide concentrating mechanism protein”, preferably being encoded by a gene comprising the nucleic acid sequence SEQ ID NO. 52634, preferably the coding region thereof, conferred the production of or the increase in hexadecadienoic acid compared with the wild type control.

The nucleic acid sequence of Sll1031 from Synechocystis sp., e.g. as shown in the respective line in column 5 of Table I, application no. 12, has been published in C. And the activity of the gene product thereof is the activity of carbon dioxide concentrating mechanism protein.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “carbon dioxide concentrating mechanism protein”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said Sll1031, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Sll1031, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Sll1031, or a functional equivalent or a homolog thereof as depicted in column 8 of Table II, application no. 12, preferably a homolog or functional equivalent as depicted in column 8 of Table II B, application no. 12, and being depicted in the same respective line as said Sll1031, and preferably the activity is increased mitochondrial, whereby the respective line discloses in column 7 the fine chemical hexadecatrienoic acid.

Accordingly, in one embodiment, the molecule which activity is to be increased in the process of the invention is the gene product with an activity as a “carbon dioxide concentrating mechanism protein”, preferably it is the molecule of section (a) or (b) of this paragraph.

In particular, it was observed that in plants, especially in Arabidopsis thaliana, increasing or generating the activity of a gene product mitochondrial with the activity of a “carbon dioxide concentrating mechanism protein”, preferably being encoded by a gene comprising the nucleic acid sequence SEQ ID NO. 52634, preferably the coding region thereof, conferred the production of or the increase in hexadecatrienoic acid compared with the wild type control.

The nucleic acid sequence of Sll1917 from Synechocystis sp., e.g. as shown in the respective line in column 5 of Table I, application no. 12, has been published in C. And the activity of the gene product thereof is the activity of coproporphyrinogen III oxidase.

Accordingly, in one embodiment, the process of the present invention for producing cerebronic acid in a non-human organism, like a microorganism or a plant or a part thereof, comprises increasing or generating the activity of a gene product with the activity of a gene product conferring the activity of “coproporphyrinogen III oxidase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical cerebronic acid), application no. 12, preferably the coding region thereof, or a homolog or a fragment thereof, and being depicted in the same respective line as said Sll1917, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Sll1917, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Sll1917, or a functional equivalent or a homolog thereof as depicted in column 8 of Table II, application no. 12, preferably a homolog or functional equivalent as depicted in column 8 of Table II B, application no. 12, and being depicted in the same respective line as said Sll1917, and preferably the activity is increased mitochondrial, whereby the respective line discloses in column 7 the fine chemical cerebronic acid.

Accordingly, in one embodiment, the molecule which activity is to be increased in the process of the invention is the gene product with an activity as a “coproporphyrinogen III oxidase”, preferably it is the molecule of section (a) or (b) of this paragraph.

In particular, it was observed that in plants, especially in Arabidopsis thaliana, increasing or generating the activity of a gene product mitochondrial with the activity of a “coproporphyrinogen III oxidase”, preferably being encoded by a gene comprising the nucleic acid sequence SEQ ID NO. 11471, preferably the coding region thereof, conferred the production of or the increase in cerebronic acid compared with the wild type control.

The nucleic acid sequence of Sll1917 from Synechocystis sp., e.g. as shown in the respective line in column 5 of Table I, application no. 12, has been published in C. And the activity of the gene product thereof is the activity of coproporphyrinogen III oxidase.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “coproporphyrinogen III oxidase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said Sll1917, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Sll1917, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Sll1917, or a functional equivalent or a homolog thereof as depicted in column 8 of Table II, application no. 12, preferably a homolog or functional equivalent as depicted in column 8 of Table II B, application no. 12, and being depicted in the same respective line as said SI11917, and preferably the activity is increased mitochondrial, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “coproporphyrinogen III oxidase”, preferably it is the molecule of section (a) or (b) of this paragraph.

In particular, it was observed that in plants, especially in Arabidopsis thaliana, increasing or generating the activity of a gene product mitochondrial with the activity of a “coproporphyrinogen III oxidase”, preferably being encoded by a gene comprising the nucleic acid sequence SEQ ID NO. 11471, preferably the coding region thereof, conferred the production of or the increase in 2-hydroxytetracosenoic acid (preferably 2-hydroxynervonic acid) compared with the wild type control.

The nucleic acid sequence of Slr0338 from Synechocystis sp., e.g. as shown in the respective line in column 5 of Table I, application no. 12, has been published in C. And the activity of the gene product thereof is the activity of oxidoreductase.

Accordingly, in one embodiment, the process of the present invention for producing 2-hydroxypalmitic acid in a non-human organism, like a microorganism or a plant or a part thereof, comprises increasing or generating the activity of a gene product with the activity of a gene product conferring the activity of “oxidoreductase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxypalmitic acid), application no. 12, preferably the coding region thereof, or a homolog or a fragment thereof, and being depicted in the same respective line as said Slr0338, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Slr0338, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Slr0338, or a functional equivalent or a homolog thereof as depicted in column 8 of Table II, application no. 12, preferably a homolog or functional equivalent as depicted in column 8 of Table II B, application no. 12, and being depicted in the same respective line as said Slr0338, and preferably the activity is increased non-targeted, whereby the respective line discloses in column 7 the fine chemical 2-hydroxypalmitic acid.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 11990, preferably the coding region thereof, conferred the production of or the increase in 2-hydroxypalmitic acid compared with the wild type control.

The nucleic acid sequence of Slr0597 from Synechocystis sp., e.g. as shown in the respective line in column 5 of Table I, application no. 12, has been published in C. And the activity of the gene product thereof is the activity of bifunctional purine biosynthesis protein.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “bifunctional purine biosynthesis protein”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said Slr0597, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Slr0597, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Slr0597, or a functional equivalent or a homolog thereof as depicted in column 8 of Table II, application no. 12, preferably a homolog or functional equivalent as depicted in column 8 of Table II B, application no. 12, and being depicted in the same respective line as said Slr0597, and preferably the activity is increased plastidic, whereby the respective line discloses in column 7 the fine chemical hexadecatrienoic acid.

Accordingly, in one embodiment, the molecule which activity is to be increased in the process of the invention is the gene product with an activity as a “bifunctional purine biosynthesis protein”, preferably it is the molecule of section (a) or (b) of this paragraph.

In particular, it was observed that in plants, especially in Arabidopsis thaliana, increasing or generating the activity of a gene product plastidic with the activity of a “bifunctional purine biosynthesis protein”, preferably being encoded by a gene comprising the nucleic acid sequence SEQ ID NO. 56153, preferably the coding region thereof, conferred the production of or the increase in hexadecatrienoic acid compared with the wild type control.

The nucleic acid sequence of Slr0721 from Synechocystis sp., e.g. as shown in the respective line in column 5 of Table I, application no. 12, has been published in C. And the activity of the gene product thereof is the activity of malate dehydrogenase.

Accordingly, in one embodiment, the process of the present invention for producing cerebronic acid in a non-human organism, like a microorganism or a plant or a part thereof, comprises increasing or generating the activity of a gene product with the activity of a gene product conferring the activity of “malate dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical cerebronic acid), application no. 12, preferably the coding region thereof, or a homolog or a fragment thereof, and being depicted in the same respective line as said Slr0721, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Slr0721, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Slr0721, or a functional equivalent or a homolog thereof as depicted in column 8 of Table II, application no. 12, preferably a homolog or functional equivalent as depicted in column 8 of Table II B, application no. 12, and being depicted in the same respective line as said Slr0721, and preferably the activity is increased plastidic, whereby the respective line discloses in column 7 the fine chemical cerebronic acid.

Accordingly, in one embodiment, the molecule which activity is to be increased in the process of the invention is the gene product with an activity as a “malate dehydrogenase”, preferably it is the molecule of section (a) or (b) of this paragraph.

In particular, it was observed that in plants, especially in Arabidopsis thaliana, increasing or generating the activity of a gene product plastidic with the activity of a “malate dehydrogenase”, preferably being encoded by a gene comprising the nucleic acid sequence SEQ ID NO. 87397, preferably the coding region thereof, conferred the production of or the increase in cerebronic acid compared with the wild type control.

The nucleic acid sequence of Slr0879 from Synechocystis sp., e.g. as shown in the respective line in column 5 of Table I, application no. 12, has been published in C. And the activity of the gene product thereof is the activity of glycine cleavage system H protein.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “glycine cleavage system H protein”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said Slr0879, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Slr0879, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Slr0879, or a functional equivalent or a homolog thereof as depicted in column 8 of Table II, application no. 12, preferably a homolog or functional equivalent as depicted in column 8 of Table II B, application no. 12, and being depicted in the same respective line as said Slr0879, and preferably the activity is increased mitochondrial, whereby the respective line discloses in column 7 the fine chemical hexadecatrienoic acid.

Accordingly, in one embodiment, the molecule which activity is to be increased in the process of the invention is the gene product with an activity as a “glycine cleavage system H protein”, preferably it is the molecule of section (a) or (b) of this paragraph.

In particular, it was observed that in plants, especially in Arabidopsis thaliana, increasing or generating the activity of a gene product mitochondrial with the activity of a “glycine cleavage system H protein”, preferably being encoded by a gene comprising the nucleic acid sequence SEQ ID NO. 127183, preferably the coding region thereof, conferred the production of or the increase in hexadecatrienoic acid compared with the wild type control.

The nucleic acid sequence of Slr0949 from Synechocystis sp., e.g. as shown in the respective line in column 5 of Table I, application no. 12, has been published in C. And the activity of the gene product thereof is the activity of permease protein of ABC transporter.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “permease protein of ABC transporter”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said Slr0949, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Slr0949, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Slr0949, or a functional equivalent or a homolog thereof as depicted in column 8 of Table II, application no. 12, preferably a homolog or functional equivalent as depicted in column 8 of Table II B, application no. 12, and being depicted in the same respective line as said Slr0949, and preferably the activity is increased mitochondrial, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “permease protein of ABC transporter”, preferably it is the molecule of section (a) or (b) of this paragraph.

In particular, it was observed that in plants, especially in Arabidopsis thaliana, increasing or generating the activity of a gene product mitochondrial with the activity of a “permease protein of ABC transporter”, preferably being encoded by a gene comprising the nucleic acid sequence SEQ ID NO. 119222, preferably the coding region thereof, conferred the production of or the increase in 2-hydroxytetracosenoic acid (preferably 2-hydroxynervonic acid) compared with the wild type control.

The nucleic acid sequence of Slr0966 from Synechocystis sp., e.g. as shown in the respective line in column 5 of Table I, application no. 12, has been published in C. And the activity of the gene product thereof is the activity of tryptophan synthase alpha chain.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “tryptophan synthase alpha chain”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said Slr0966, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Slr0966, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Slr0966, or a functional equivalent or a homolog thereof as depicted in column 8 of Table II, application no. 12, preferably a homolog or functional equivalent as depicted in column 8 of Table II B, application no. 12, and being depicted in the same respective line as said Slr0966, and preferably the activity is increased plastidic, whereby the respective line discloses in column 7 the fine chemical hexadecadienoic acid.

Accordingly, in one embodiment, the molecule which activity is to be increased in the process of the invention is the gene product with an activity as a “tryptophan synthase alpha chain”, preferably it is the molecule of section (a) or (b) of this paragraph.

In particular, it was observed that in plants, especially in Arabidopsis thaliana, increasing or generating the activity of a gene product plastidic with the activity of a “tryptophan synthase alpha chain”, preferably being encoded by a gene comprising the nucleic acid sequence SEQ ID NO. 87655, preferably the coding region thereof, conferred the production of or the increase in hexadecadienoic acid compared with the wild type control.

The nucleic acid sequence of Slr1096 from Synechocystis sp., e.g. as shown in the respective line in column 5 of Table I, application no. 12, has been published in C. And the activity of the gene product thereof is the activity of dihydrolipoamide dehydrogenase.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “dihydrolipoamide dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said Slr1096, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Slr1096, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Slr1096, or a functional equivalent or a homolog thereof as depicted in column 8 of Table II, application no. 12, preferably a homolog or functional equivalent as depicted in column 8 of Table II B, application no. 12, and being depicted in the same respective line as said Slr1096, and preferably the activity is increased non-targeted, whereby the respective line discloses in column 7 the fine chemical 2-hydroxybehenic acid.

Accordingly, in one embodiment, the molecule which activity is to be increased in the process of the invention is the gene product with an activity as a “dihydrolipoamide dehydrogenase”, preferably it is the molecule of section (a) or (b) of this paragraph.

In particular, it was observed that in plants, especially in Arabidopsis thaliana, increasing or generating the activity of a gene product non-targeted with the activity of a “dihydrolipoamide dehydrogenase”, preferably being encoded by a gene comprising the nucleic acid sequence SEQ ID NO. 57734, preferably the coding region thereof, conferred the production of or the increase in 2-hydroxybehenic acid compared with the wild type control.

The nucleic acid sequence of Slr1250 from Synechocystis sp., e.g. as shown in the respective line in column 5 of Table I, application no. 12, has been published in C. And the activity of the gene product thereof is the activity of phosphate import ATP-binding protein.

Accordingly, in one embodiment, the process of the present invention for producing 2-hydroxypalmitic acid in a non-human organism, like a microorganism or a plant or a part thereof, comprises increasing or generating the activity of a gene product with the activity of a gene product conferring the activity of “phosphate import ATP-binding protein”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxypalmitic acid), application no. 12, preferably the coding region thereof, or a homolog or a fragment thereof, and being depicted in the same respective line as said Slr1250, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Slr1250, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Slr1250, or a functional equivalent or a homolog thereof as depicted in column 8 of Table II, application no. 12, preferably a homolog or functional equivalent as depicted in column 8 of Table II B, application no. 12, and being depicted in the same respective line as said Slr1250, and preferably the activity is increased mitochondrial, whereby the respective line discloses in column 7 the fine chemical 2-hydroxypalmitic acid.

Accordingly, in one embodiment, the molecule which activity is to be increased in the process of the invention is the gene product with an activity as a “phosphate import ATP-binding protein”, preferably it is the molecule of section (a) or (b) of this paragraph.

In particular, it was observed that in plants, especially in Arabidopsis thaliana, increasing or generating the activity of a gene product mitochondrial with the activity of a “phosphate import ATP-binding protein”, preferably being encoded by a gene comprising the nucleic acid sequence SEQ ID NO. 116460, preferably the coding region thereof, conferred the production of or the increase in 2-hydroxypalmitic acid compared with the wild type control.

The nucleic acid sequence of Slr2035 from Synechocystis sp., e.g. as shown in the respective line in column 5 of Table I, application no. 12, has been published in C. And the activity of the gene product thereof is the activity of glutamate 5-kinase.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “glutamate 5-kinase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said Slr2035, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Slr2035, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Slr2035, or a functional equivalent or a homolog thereof as depicted in column 8 of Table II, application no. 12, preferably a homolog or functional equivalent as depicted in column 8 of Table II B, application no. 12, and being depicted in the same respective line as said Slr2035, and preferably the activity is increased mitochondrial, whereby the respective line discloses in column 7 the fine chemical hexadecatrienoic acid.

Accordingly, in one embodiment, the molecule which activity is to be increased in the process of the invention is the gene product with an activity as a “glutamate 5-kinase”, preferably it is the molecule of section (a) or (b) of this paragraph.

In particular, it was observed that in plants, especially in Arabidopsis thaliana, increasing or generating the activity of a gene product mitochondrial with the activity of a “glutamate 5-kinase”, preferably being encoded by a gene comprising the nucleic acid sequence SEQ ID NO. 127521, preferably the coding region thereof, conferred the production of or the increase in hexadecatrienoic acid compared with the wild type control.

The nucleic acid sequence of Slr2072 from Synechocystis sp., e.g. as shown in the respective line in column 5 of Table I, application no. 12, has been published in C. And the activity of the gene product thereof is the activity of threonine dehydratase.

Accordingly, in one embodiment, the process of the present invention for producing cerebronic acid in a non-human organism, like a microorganism or a plant or a part thereof, comprises increasing or generating the activity of a gene product with the activity of a gene product conferring the activity of “threonine dehydratase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical cerebronic acid), application no. 12, preferably the coding region thereof, or a homolog or a fragment thereof, and being depicted in the same respective line as said Slr2072, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Slr2072, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Slr2072, or a functional equivalent or a homolog thereof as depicted in column 8 of Table II, application no. 12, preferably a homolog or functional equivalent as depicted in column 8 of Table II B, application no. 12, and being depicted in the same respective line as said Slr2072, and preferably the activity is increased mitochondrial, whereby the respective line discloses in column 7 the fine chemical cerebronic acid.

Accordingly, in one embodiment, the molecule which activity is to be increased in the process of the invention is the gene product with an activity as a “threonine dehydratase”, preferably it is the molecule of section (a) or (b) of this paragraph.

In particular, it was observed that in plants, especially in Arabidopsis thaliana, increasing or generating the activity of a gene product mitochondrial with the activity of a “threonine dehydratase”, preferably being encoded by a gene comprising the nucleic acid sequence SEQ ID NO. 12341, preferably the coding region thereof, conferred the production of or the increase in cerebronic acid compared with the wild type control.

The nucleic acid sequence of Slr2072 from Synechocystis sp., e.g. as shown in the respective line in column 5 of Table I, application no. 12, has been published in C. And the activity of the gene product thereof is the activity of threonine dehydratase.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “threonine dehydratase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said Slr2072, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Slr2072, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Slr2072, or a functional equivalent or a homolog thereof as depicted in column 8 of Table II, application no. 12, preferably a homolog or functional equivalent as depicted in column 8 of Table II B, application no. 12, and being depicted in the same respective line as said Slr2072, and preferably the activity is increased mitochondrial, whereby the respective line discloses in column 7 the fine chemical hexadecatrienoic acid.

Accordingly, in one embodiment, the molecule which activity is to be increased in the process of the invention is the gene product with an activity as a “threonine dehydratase”, preferably it is the molecule of section (a) or (b) of this paragraph.

In particular, it was observed that in plants, especially in Arabidopsis thaliana, increasing or generating the activity of a gene product mitochondrial with the activity of a “threonine dehydratase”, preferably being encoded by a gene comprising the nucleic acid sequence SEQ ID NO. 12341, preferably the coding region thereof, conferred the production of or the increase in hexadecatrienoic acid compared with the wild type control.

The nucleic acid sequence of Slr2072 from Synechocystis sp., e.g. as shown in the respective line in column 5 of Table I, application no. 12, has been published in C. And the activity of the gene product thereof is the activity of threonine dehydratase.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “threonine dehydratase”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said Slr2072, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Slr2072, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Slr2072, or a functional equivalent or a homolog thereof as depicted in column 8 of Table II, application no. 12, preferably a homolog or functional equivalent as depicted in column 8 of Table II B, application no. 12, and being depicted in the same respective line as said Slr2072, and preferably the activity is increased plastidic, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “threonine dehydratase”, preferably it is the molecule of section (a) or (b) of this paragraph.

In particular, it was observed that in plants, especially in Arabidopsis thaliana, increasing or generating the activity of a gene product plastidic with the activity of a “threonine dehydratase”, preferably being encoded by a gene comprising the nucleic acid sequence SEQ ID NO. 12341, preferably the coding region thereof, conferred the production of or the increase in 2-hydroxytetracosenoic acid (preferably 2-hydroxynervonic acid) compared with the wild type control.

The nucleic acid sequence of SsI3044 from Synechocystis sp., e.g. as shown in the respective line in column 5 of Table I, application no. 12, has been published in C. And the activity of the gene product thereof is the activity of ferredoxin.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “ferredoxin”, especially from Synechocystis sp. or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said Ssl3044, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ss13044, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ssl3044, or a functional equivalent or a homolog thereof as depicted in column 8 of Table II, application no. 12, preferably a homolog or functional equivalent as depicted in column 8 of Table II B, application no. 12, and being depicted in the same respective line as said Ss13044, and preferably the activity is increased plastidic, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “ferredoxin”, preferably it is the molecule of section (a) or (b) of this paragraph.

In particular, it was observed that in plants, especially in Arabidopsis thaliana, increasing or generating the activity of a gene product plastidic with the activity of a “ferredoxin”, preferably being encoded by a gene comprising the nucleic acid sequence SEQ ID NO. 128024, preferably the coding region thereof, conferred the production of or the increase in 2-hydroxytetracosenoic acid (preferably 2-hydroxynervonic acid) compared with the wild type control.

The nucleic acid sequence of TTC0019 from Thermus thermophilus, e.g. as shown in the respective line in column 5 of Table I, application no. 12, has been published in E. And the activity of the gene product thereof is the activity of Sec-independent protein translocase subunit.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “Sec-independent protein translocase subunit”, especially from Thermus thermophilus or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said TTC0019, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said TTC0019, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said TTC0019, or a functional equivalent or a homolog thereof as depicted in column 8 of Table II, application no. 12, preferably a homolog or functional equivalent as depicted in column 8 of Table II B, application no. 12, and being depicted in the same respective line as said TTC0019, and preferably the activity is increased non-targeted, whereby the respective line discloses in column 7 the fine chemical 2-hydroxybehenic acid.

Accordingly, in one embodiment, the molecule which activity is to be increased in the process of the invention is the gene product with an activity as a “Sec-independent protein translocase subunit”, preferably it is the molecule of section (a) or (b) of this paragraph.

In particular, it was observed that in plants, especially in Arabidopsis thaliana, increasing or generating the activity of a gene product non-targeted with the activity of a “Sec-independent protein translocase subunit”, preferably being encoded by a gene comprising the nucleic acid sequence SEQ ID NO. 12698, preferably the coding region thereof, conferred the production of or the increase in 2-hydroxybehenic acid compared with the wild type control.

The nucleic acid sequence of TTC0337 from Thermus thermophilus, e.g. as shown in the respective line in column 5 of Table I, application no. 12, has been published in E. And the activity of the gene product thereof is the activity of amino acid ABC transporter permease protein.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “amino acid ABC transporter permease protein”, especially from Thermus thermophilus or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said TTC0337, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said TTC0337, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said TTC0337, or a functional equivalent or a homolog thereof as depicted in column 8 of Table II, application no. 12, preferably a homolog or functional equivalent as depicted in column 8 of Table II B, application no. 12, and being depicted in the same respective line as said TTC0337, and preferably the activity is increased non-targeted, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “amino acid ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “amino acid ABC transporter permease protein”, preferably being encoded by a gene comprising the nucleic acid sequence SEQ ID NO. 61070, preferably the coding region thereof, conferred the production of or the increase in 2-hydroxytetracosenoic acid (preferably 2-hydroxynervonic acid) compared with the wild type control.

The nucleic acid sequence of TTC0917 from Thermus thermophilus, e.g. as shown in the respective line in column 5 of Table I, application no. 12, has been published in E. And the activity of the gene product thereof is the activity of metal-dependent hydrolase.

Accordingly, in one embodiment, the process of the present invention for producing cerebronic acid in a non-human organism, like a microorganism or a plant or a part thereof, comprises increasing or generating the activity of a gene product with the activity of a gene product conferring the activity of “metal-dependent hydrolase”, especially from Thermus thermophilus or its functional equivalent or its homolog, e.g. the increase of
(a) a gene product of a gene comprising the nucleic acid molecule as shown in the respective line in column 5 of Table I (whereby the respective line discloses in column 7 the fine chemical cerebronic acid), application no. 12, preferably the coding region thereof, or a homolog or a fragment thereof, and being depicted in the same respective line as said TTC0917, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said TTC0917, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said TTC0917, or a functional equivalent or a homolog thereof as depicted in column 8 of Table II, application no. 12, preferably a homolog or functional equivalent as depicted in column 8 of Table II B, application no. 12, and being depicted in the same respective line as said TTC0917, and preferably the activity is increased non-targeted, whereby the respective line discloses in column 7 the fine chemical cerebronic acid.

Accordingly, in one embodiment, the molecule which activity is to be increased in the process of the invention is the gene product with an activity as a “metal-dependent hydrolase”, preferably it is the molecule of section (a) or (b) of this paragraph.

In particular, it was observed that in plants, especially in Arabidopsis thaliana, increasing or generating the activity of a gene product non-targeted with the activity of a “metal-dependent hydrolase”, preferably being encoded by a gene comprising the nucleic acid sequence SEQ ID NO. 61553, preferably the coding region thereof, conferred the production of or the increase in cerebronic acid compared with the wild type control.

The nucleic acid sequence of TTC0917 from Thermus thermophilus, e.g. as shown in the respective line in column 5 of Table I, application no. 12, has been published in E. And the activity of the gene product thereof is the activity of metal-dependent hydrolase.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “metal-dependent hydrolase”, especially from Thermus thermophilus or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said TTC0917, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said TTC0917, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said TTC0917, or a functional equivalent or a homolog thereof as depicted in column 8 of Table II, application no. 12, preferably a homolog or functional equivalent as depicted in column 8 of Table II B, application no. 12, and being depicted in the same respective line as said TTC0917, and preferably the activity is increased non-targeted, whereby the respective line discloses in column 7 the fine chemical hexadecadienoic acid.

Accordingly, in one embodiment, the molecule which activity is to be increased in the process of the invention is the gene product with an activity as a “metal-dependent hydrolase”, preferably it is the molecule of section (a) or (b) of this paragraph.

In particular, it was observed that in plants, especially in Arabidopsis thaliana, increasing or generating the activity of a gene product non-targeted with the activity of a “metal-dependent hydrolase”, preferably being encoded by a gene comprising the nucleic acid sequence SEQ ID NO. 61553, preferably the coding region thereof, conferred the production of or the increase in hexadecadienoic acid compared with the wild type control.

The nucleic acid sequence of TTC1193 from Thermus thermophilus, e.g. as shown in the respective line in column 5 of Table I, application no. 12, has been published in E. And the activity of the gene product thereof is the activity of multiple antibiotic resistance protein.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “multiple antibiotic resistance protein”, especially from Thermus thermophilus or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said TTC1193, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said TTC1193, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said TTC1193, or a functional equivalent or a homolog thereof as depicted in column 8 of Table II, application no. 12, preferably a homolog or functional equivalent as depicted in column 8 of Table II B, application no. 12, and being depicted in the same respective line as said TTC1193, and preferably the activity is increased non-targeted, whereby the respective line discloses in column 7 the fine chemical 2-hydroxybehenic acid.

Accordingly, in one embodiment, the molecule which activity is to be increased in the process of the invention is the gene product with an activity as a “multiple antibiotic resistance protein”, preferably it is the molecule of section (a) or (b) of this paragraph.

In particular, it was observed that in plants, especially in Arabidopsis thaliana, increasing or generating the activity of a gene product non-targeted with the activity of a “multiple antibiotic resistance protein”, preferably being encoded by a gene comprising the nucleic acid sequence SEQ ID NO. 61723, preferably the coding region thereof, conferred the production of or the increase in 2-hydroxybehenic acid compared with the wild type control.

The nucleic acid sequence of TTC1386 from Thermus thermophilus, e.g. as shown in the respective line in column 5 of Table I, application no. 12, has been published in E. And the activity of the gene product thereof is the activity of TTC1386-protein.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “TTC1386-protein”, especially from Thermus thermophilus or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said TTC1386, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said TTC1386, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said TTC1386, or a functional equivalent or a homolog thereof as depicted in column 8 of Table II, application no. 12, preferably a homolog or functional equivalent as depicted in column 8 of Table II B, application no. 12, and being depicted in the same respective line as said TTC1386, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “TTC1386-protein”, preferably it is the molecule of section (a) or (b) of this paragraph.

In particular, it was observed that in plants, especially in Arabidopsis thaliana, increasing or generating the activity of a gene product non-targeted with the activity of a “TTC1386-protein”, preferably being encoded by a gene comprising the nucleic acid sequence SEQ ID NO. 62079, preferably the coding region thereof, conferred the production of or the increase in hexadecatrienoic acid compared with the wild type control.

The nucleic acid sequence of TTC1550 from Thermus thermophilus, e.g. as shown in the respective line in column 5 of Table I, application no. 12, has been published in E. And the activity of the gene product thereof is the activity of homocitrate synthase.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “homocitrate synthase”, especially from Thermus thermophilus or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said TTC1550, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said TTC1550, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said TTC1550, or a functional equivalent or a homolog thereof as depicted in column 8 of Table II, application no. 12, preferably a homolog or functional equivalent as depicted in column 8 of Table II B, application no. 12, and being depicted in the same respective line as said TTC1550, and preferably the activity is increased non-targeted, whereby the respective line discloses in column 7 the fine chemical 2-hydroxybehenic acid.

Accordingly, in one embodiment, the molecule which activity is to be increased in the process of the invention is the gene product with an activity as a “homocitrate synthase”, preferably it is the molecule of section (a) or (b) of this paragraph.

In particular, it was observed that in plants, especially in Arabidopsis thaliana, increasing or generating the activity of a gene product non-targeted with the activity of a “homocitrate synthase”, preferably being encoded by a gene comprising the nucleic acid sequence SEQ ID NO. 12974, preferably the coding region thereof, conferred the production of or the increase in 2-hydroxybehenic acid compared with the wild type control.

The nucleic acid sequence of Yar019c from Saccharomyces cerevisiae, e.g. as shown in the respective line in column 5 of Table I, application no. 12, has been published in A. And the activity of the gene product thereof is the activity of protein kinase.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “protein kinase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said Yar019c, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Yar019c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Yar019c, or a functional equivalent or a homolog thereof as depicted in column 8 of Table II, application no. 12, preferably a homolog or functional equivalent as depicted in column 8 of Table II B, application no. 12, and being depicted in the same respective line as said Yar019c, and preferably the activity is increased non-targeted, whereby the respective line discloses in column 7 the fine chemical hexadecadienoic acid.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 128139, preferably the coding region thereof, conferred the production of or the increase in hexadecadienoic acid compared with the wild type control.

The nucleic acid sequence of Yar019c from Saccharomyces cerevisiae, e.g. as shown in the respective line in column 5 of Table I, application no. 12, has been published in A. And the activity of the gene product thereof is the activity of protein kinase.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “protein kinase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said Yar019c, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Yar019c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Yar019c, or a functional equivalent or a homolog thereof as depicted in column 8 of Table II, application no. 12, preferably a homolog or functional equivalent as depicted in column 8 of Table II B, application no. 12, and being depicted in the same respective line as said Yar019c, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 128139, preferably the coding region thereof, conferred the production of or the increase in hexadecatrienoic acid compared with the wild type control.

The nucleic acid sequence of Ybl021 c from Saccharomyces cerevisiae, e.g. as shown in the respective line in column 5 of Table I, application no. 12, has been published in A. And the activity of the gene product thereof is the activity of transcription factor.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “transcription factor”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said Ybl021 c, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ybl021c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ybl021c, or a functional equivalent or a homolog thereof as depicted in column 8 of Table II, application no. 12, preferably a homolog or functional equivalent as depicted in column 8 of Table II B, application no. 12, and being depicted in the same respective line as said Ybl021c, and preferably the activity is increased non-targeted, whereby the respective line discloses in column 7 the fine chemical hexadecadienoic acid.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 62524, preferably the coding region thereof, conferred the production of or the increase in hexadecadienoic acid compared with the wild type control.

The nucleic acid sequence of Ybl021 c from Saccharomyces cerevisiae, e.g. as shown in the respective line in column 5 of Table I, application no. 12, has been published in A. And the activity of the gene product thereof is the activity of transcription factor.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “transcription factor”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said Ybl021 c, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ybl021c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ybl021c, or a functional equivalent or a homolog thereof as depicted in column 8 of Table II, application no. 12, preferably a homolog or functional equivalent as depicted in column 8 of Table II B, application no. 12, and being depicted in the same respective line as said Ybl021c, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 62524, preferably the coding region thereof, conferred the production of or the increase in hexadecatrienoic acid compared with the wild type control.

The nucleic acid sequence of Ybr160w from Saccharomyces cerevisiae, e.g. as shown in the respective line in column 5 of Table I, application no. 12, has been published in A. And the activity of the gene product thereof is the activity of cell division control protein.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “cell division control protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said Ybr160w, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ybr160w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ybr160w, or a functional equivalent or a homolog thereof as depicted in column 8 of Table II, application no. 12, preferably a homolog or functional equivalent as depicted in column 8 of Table II B, application no. 12, and being depicted in the same respective line as said Ybr160w, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “cell division control protein”, preferably it is the molecule of section (a) or (b) of this paragraph.

In particular, it was observed that in plants, especially in Arabidopsis thaliana, increasing or generating the activity of a gene product non-targeted with the activity of a “cell division control protein”, preferably being encoded by a gene comprising the nucleic acid sequence SEQ ID NO. 62717, preferably the coding region thereof, conferred the production of or the increase in hexadecatrienoic acid compared with the wild type control.

The nucleic acid sequence of Ycl027w from Saccharomyces cerevisiae, e.g. as shown in the respective line in column 5 of Table I, application no. 12, has been published in A. And the activity of the gene product thereof is the activity of ycl027w-protein.

Accordingly, in one embodiment, the process of the present invention for producing cerebronic acid in a non-human organism, like a microorganism or a plant or a part thereof, comprises increasing or generating the activity of a gene product with the activity of a gene product conferring the activity of “ycl027w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical cerebronic acid), application no. 12, preferably the coding region thereof, or a homolog or a fragment thereof, and being depicted in the same respective line as said Ycl027w, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ycl027w, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ycl027w, or a functional equivalent or a homolog thereof as depicted in column 8 of Table II, application no. 12, preferably a homolog or functional equivalent as depicted in column 8 of Table II B, application no. 12, and being depicted in the same respective line as said Ycl027w, and preferably the activity is increased plastidic, whereby the respective line discloses in column 7 the fine chemical cerebronic acid.

Accordingly, in one embodiment, the molecule which activity is to be increased in the process of the invention is the gene product with an activity as a “ycl027w-protein”, preferably it is the molecule of section (a) or (b) of this paragraph.

In particular, it was observed that in plants, especially in Arabidopsis thaliana, increasing or generating the activity of a gene product plastidic with the activity of a “ycl027w-protein”, preferably being encoded by a gene comprising the nucleic acid sequence SEQ ID NO. 96980, preferably the coding region thereof, conferred the production of or the increase in cerebronic acid compared with the wild type control.

The nucleic acid sequence of Ycl027w from Saccharomyces cerevisiae, e.g. as shown in the respective line in column 5 of Table I, application no. 12, has been published in A. And the activity of the gene product thereof is the activity of ycl027w-protein.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “ycl027w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said Ycl027w, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ycl027w, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ycl027w, or a functional equivalent or a homolog thereof as depicted in column 8 of Table II, application no. 12, preferably a homolog or functional equivalent as depicted in column 8 of Table II B, application no. 12, and being depicted in the same respective line as said Ycl027w, and preferably the activity is increased plastidic, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “ycl027w-protein”, preferably it is the molecule of section (a) or (b) of this paragraph.

In particular, it was observed that in plants, especially in Arabidopsis thaliana, increasing or generating the activity of a gene product plastidic with the activity of a “ycl027w-protein”, preferably being encoded by a gene comprising the nucleic acid sequence SEQ ID NO. 96980, preferably the coding region thereof, conferred the production of or the increase in 2-hydroxytetracosenoic acid (preferably 2-hydroxynervonic acid) compared with the wild type control.

The nucleic acid sequence of Ycl049c from Saccharomyces cerevisiae, e.g. as shown in the respective line in column 5 of Table I, application no. 12, has been published in A. And the activity of the gene product thereof is the activity of ycl049c-protein.

Accordingly, in one embodiment, the process of the present invention for producing 2-hydroxypalmitic acid in a non-human organism, like a microorganism or a plant or a part thereof, comprises increasing or generating the activity of a gene product with the activity of a gene product conferring the activity of “ycl049c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxypalmitic acid), application no. 12, preferably the coding region thereof, or a homolog or a fragment thereof, and being depicted in the same respective line as said Ycl049c, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ycl049c, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ycl049c, or a functional equivalent or a homolog thereof as depicted in column 8 of Table II, application no. 12, preferably a homolog or functional equivalent as depicted in column 8 of Table II B, application no. 12, and being depicted in the same respective line as said Ycl049c, and preferably the activity is increased plastidic, whereby the respective line discloses in column 7 the fine chemical 2-hydroxypalmitic acid.

Accordingly, in one embodiment, the molecule which activity is to be increased in the process of the invention is the gene product with an activity as a “ycl049c-protein”, preferably it is the molecule of section (a) or (b) of this paragraph.

In particular, it was observed that in plants, especially in Arabidopsis thaliana, increasing or generating the activity of a gene product plastidic with the activity of a “ycl049c-protein”, preferably being encoded by a gene comprising the nucleic acid sequence SEQ ID NO. 113220, preferably the coding region thereof, conferred the production of or the increase in 2-hydroxypalmitic acid compared with the wild type control.

The nucleic acid sequence of Ydl022w from Saccharomyces cerevisiae, e.g. as shown in the respective line in column 5 of Table I, application no. 12, has been published in A. And the activity of the gene product thereof is the activity of glycerol-3-phosphate dehydrogenase.

Accordingly, in one embodiment, the process of the present invention for producing oleic acid in a non-human organism, like a microorganism or a plant or a part thereof, comprises increasing or generating the activity of a gene product with the activity of a gene product conferring the activity of “glycerol-3-phosphate dehydrogenase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical oleic acid), application no. 12, preferably the coding region thereof, or a homolog or a fragment thereof, and being depicted in the same respective line as said Ydl022w, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ydl022w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ydl022w, or a functional equivalent or a homolog thereof as depicted in column 8 of Table II, application no. 12, preferably a homolog or functional equivalent as depicted in column 8 of Table II B, application no. 12, and being depicted in the same respective line as said Ydl022w, and preferably the activity is increased non-targeted, whereby the respective line discloses in column 7 the fine chemical oleic acid.

Accordingly, in one embodiment, the molecule which activity is to be increased in the process of the invention is the gene product with an activity as a “glycerol-3-phosphate dehydrogenase”, preferably it is the molecule of section (a) or (b) of this paragraph.

In particular, it was observed that in plants, especially in Arabidopsis thaliana, increasing or generating the activity of a gene product non-targeted with the activity of a “glycerol-3-phosphate dehydrogenase”, preferably being encoded by a gene comprising the nucleic acid sequence SEQ ID NO. 119408, preferably the coding region thereof, conferred the production of or the increase in oleic acid compared with the wild type control.

The nucleic acid sequence of Ydl066w from Saccharomyces cerevisiae, e.g. as shown in the respective line in column 5 of Table I, application no. 12, has been published in A. And the activity of the gene product thereof is the activity of isocitrate dehydrogenase.

Accordingly, in one embodiment, the process of the present invention for producing eicosanoic acid in a non-human organism, like a microorganism or a plant or a part thereof, comprises increasing or generating the activity of a gene product with the activity of a gene product conferring the activity of “isocitrate dehydrogenase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical eicosanoic acid), application no. 12, preferably the coding region thereof, or a homolog or a fragment thereof, and being depicted in the same respective line as said Ydl066w, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ydl066w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ydl066w, or a functional equivalent or a homolog thereof as depicted in column 8 of Table II, application no. 12, preferably a homolog or functional equivalent as depicted in column 8 of Table II B, application no. 12, and being depicted in the same respective line as said Ydl066w, and preferably the activity is increased non-targeted, whereby the respective line discloses in column 7 the fine chemical eicosanoic acid.

Accordingly, in one embodiment, the molecule which activity is to be increased in the process of the invention is the gene product with an activity as a “isocitrate dehydrogenase”, preferably it is the molecule of section (a) or (b) of this paragraph.

In particular, it was observed that in plants, especially in Arabidopsis thaliana, increasing or generating the activity of a gene product non-targeted with the activity of a “isocitrate dehydrogenase”, preferably being encoded by a gene comprising the nucleic acid sequence SEQ ID NO. 122228, preferably the coding region thereof, conferred the production of or the increase in eicosanoic acid compared with the wild type control.

The nucleic acid sequence of Ydl066w from Saccharomyces cerevisiae, e.g. as shown in the respective line in column 5 of Table I, application no. 12, has been published in A. And the activity of the gene product thereof is the activity of isocitrate dehydrogenase.

Accordingly, in one embodiment, the process of the present invention for producing heptadecanoic acid in a non-human organism, like a microorganism or a plant or a part thereof, comprises increasing or generating the activity of a gene product with the activity of a gene product conferring the activity of “isocitrate dehydrogenase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical heptadecanoic acid), application no. 12, preferably the coding region thereof, or a homolog or a fragment thereof, and being depicted in the same respective line as said Ydl066w, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ydl066w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ydl066w, or a functional equivalent or a homolog thereof as depicted in column 8 of Table II, application no. 12, preferably a homolog or functional equivalent as depicted in column 8 of Table II B, application no. 12, and being depicted in the same respective line as said Ydl066w, and preferably the activity is increased non-targeted, whereby the respective line discloses in column 7 the fine chemical heptadecanoic acid.

Accordingly, in one embodiment, the molecule which activity is to be increased in the process of the invention is the gene product with an activity as a “isocitrate dehydrogenase”, preferably it is the molecule of section (a) or (b) of this paragraph.

In particular, it was observed that in plants, especially in Arabidopsis thaliana, increasing or generating the activity of a gene product non-targeted with the activity of a “isocitrate dehydrogenase”, preferably being encoded by a gene comprising the nucleic acid sequence SEQ ID NO. 122228, preferably the coding region thereof, conferred the production of or the increase in heptadecanoic acid compared with the wild type control.

The nucleic acid sequence of Ydl126c from Saccharomyces cerevisiae, e.g. as shown in the respective line in column 5 of Table I, application no. 12, has been published in A. And the activity of the gene product thereof is the activity of cell division control protein.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “cell division control protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said Ydl126c, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ydl126c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ydl126c, or a functional equivalent or a homolog thereof as depicted in column 8 of Table II, application no. 12, preferably a homolog or functional equivalent as depicted in column 8 of Table II B, application no. 12, and being depicted in the same respective line as said Ydl126c, and preferably the activity is increased non-targeted, whereby the respective line discloses in column 7 the fine chemical hexadecadienoic acid.

Accordingly, in one embodiment, the molecule which activity is to be increased in the process of the invention is the gene product with an activity as a “cell division control protein”, preferably it is the molecule of section (a) or (b) of this paragraph.

In particular, it was observed that in plants, especially in Arabidopsis thaliana, increasing or generating the activity of a gene product non-targeted with the activity of a “cell division control protein”, preferably being encoded by a gene comprising the nucleic acid sequence SEQ ID NO. 120045, preferably the coding region thereof, conferred the production of or the increase in hexadecadienoic acid compared with the wild type control.

The nucleic acid sequence of Ydl126c from Saccharomyces cerevisiae, e.g. as shown in the respective line in column 5 of Table I, application no. 12, has been published in A. And the activity of the gene product thereof is the activity of cell division control protein.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “cell division control protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said Ydl126c, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ydl126c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ydl126c, or a functional equivalent or a homolog thereof as depicted in column 8 of Table II, application no. 12, preferably a homolog or functional equivalent as depicted in column 8 of Table II B, application no. 12, and being depicted in the same respective line as said Ydl126c, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “cell division control protein”, preferably it is the molecule of section (a) or (b) of this paragraph.

In particular, it was observed that in plants, especially in Arabidopsis thaliana, increasing or generating the activity of a gene product non-targeted with the activity of a “cell division control protein”, preferably being encoded by a gene comprising the nucleic acid sequence SEQ ID NO. 120045, preferably the coding region thereof, conferred the production of or the increase in hexadecatrienoic acid compared with the wild type control.

The nucleic acid sequence of Ydl131w from Saccharomyces cerevisiae, e.g. as shown in the respective line in column 5 of Table I, application no. 12, has been published in A. And the activity of the gene product thereof is the activity of homocitrate synthase.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “homocitrate synthase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said Ydl131w, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ydl131w, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ydl131w, or a functional equivalent or a homolog thereof as depicted in column 8 of Table II, application no. 12, preferably a homolog or functional equivalent as depicted in column 8 of Table II B, application no. 12, and being depicted in the same respective line as said Ydl131w, and preferably the activity is increased plastidic, whereby the respective line discloses in column 7 the fine chemical 2-hydroxybehenic acid.

Accordingly, in one embodiment, the molecule which activity is to be increased in the process of the invention is the gene product with an activity as a “homocitrate synthase”, preferably it is the molecule of section (a) or (b) of this paragraph.

In particular, it was observed that in plants, especially in Arabidopsis thaliana, increasing or generating the activity of a gene product plastidic with the activity of a “homocitrate synthase”, preferably being encoded by a gene comprising the nucleic acid sequence SEQ ID NO. 113224, preferably the coding region thereof, conferred the production of or the increase in 2-hydroxybehenic acid compared with the wild type control.

The nucleic acid sequence of Ydr035w from Saccharomyces cerevisiae, e.g. as shown in the respective line in column 5 of Table I, application no. 12, has been published in A. And the activity of the gene product thereof is the activity of phospho-2-dehydro-3-deoxyheptonate aldolase.

Accordingly, in one embodiment, the process of the present invention for producing behenic acid in a non-human organism, like a microorganism or a plant or a part thereof, comprises increasing or generating the activity of a gene product with the activity of a gene product conferring the activity of “phospho-2-dehydro-3-deoxyheptonate aldolase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical behenic acid), application no. 12, preferably the coding region thereof, or a homolog or a fragment thereof, and being depicted in the same respective line as said Ydr035w, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ydr035w, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ydr035w, or a functional equivalent or a homolog thereof as depicted in column 8 of Table II, application no. 12, preferably a homolog or functional equivalent as depicted in column 8 of Table II B, application no. 12, and being depicted in the same respective line as said Ydr035w, and preferably the activity is increased plastidic, whereby the respective line discloses in column 7 the fine chemical behenic acid.

Accordingly, in one embodiment, the molecule which activity is to be increased in the process of the invention is the gene product with an activity as a “phospho-2-dehydro-3-deoxyheptonate aldolase”, preferably it is the molecule of section (a) or (b) of this paragraph.

In particular, it was observed that in plants, especially in Arabidopsis thaliana, increasing or generating the activity of a gene product plastidic with the activity of a “phospho-2-dehydro-3-deoxyheptonate aldolase”, preferably being encoded by a gene comprising the nucleic acid sequence SEQ ID NO. 113330, preferably the coding region thereof, conferred the production of or the increase in behenic acid compared with the wild type control.

The nucleic acid sequence of Ydr077w from Saccharomyces cerevisiae, e.g. as shown in the respective line in column 5 of Table I, application no. 12, has been published in A. And the activity of the gene product thereof is the activity of cell wall protein.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “cell wall protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said Ydr077w, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ydr077w, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ydr077w, or a functional equivalent or a homolog thereof as depicted in column 8 of Table II, application no. 12, preferably a homolog or functional equivalent as depicted in column 8 of Table II B, application no. 12, and being depicted in the same respective line as said Ydr077w, and preferably the activity is increased plastidic, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “cell wall protein”, preferably it is the molecule of section (a) or (b) of this paragraph.

In particular, it was observed that in plants, especially in Arabidopsis thaliana, increasing or generating the activity of a gene product plastidic with the activity of a “cell wall protein”, preferably being encoded by a gene comprising the nucleic acid sequence SEQ ID NO. 128148, preferably the coding region thereof, conferred the production of or the increase in 2-hydroxytetracosenoic acid (preferably 2-hydroxynervonic acid) compared with the wild type control.

The nucleic acid sequence of Ydr131c from Saccharomyces cerevisiae, e.g. as shown in the respective line in column 5 of Table I, application no. 12, has been published in A. And the activity of the gene product thereof is the activity of F-box protein.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “F-box protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said Ydr131c, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ydr131c, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ydr131c, or a functional equivalent or a homolog thereof as depicted in column 8 of Table II, application no. 12, preferably a homolog or functional equivalent as depicted in column 8 of Table II B, application no. 12, and being depicted in the same respective line as said Ydr131c, and preferably the activity is increased plastidic, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “F-box protein”, preferably it is the molecule of section (a) or (b) of this paragraph.

In particular, it was observed that in plants, especially in Arabidopsis thaliana, increasing or generating the activity of a gene product plastidic with the activity of a “F-box protein”, preferably being encoded by a gene comprising the nucleic acid sequence SEQ ID NO. 14706, preferably the coding region thereof, conferred the production of or the increase in 2-hydroxytetracosenoic acid (preferably 2-hydroxynervonic acid) compared with the wild type control.

The nucleic acid sequence of Ydr273w from Saccharomyces cerevisiae, e.g. as shown in the respective line in column 5 of Table I, application no. 12, has been published in A. And the activity of the gene product thereof is the activity of ydr273w-protein.

Accordingly, in one embodiment, the process of the present invention for producing cerebronic acid in a non-human organism, like a microorganism or a plant or a part thereof, comprises increasing or generating the activity of a gene product with the activity of a gene product conferring the activity of “ydr273w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical cerebronic acid), application no. 12, preferably the coding region thereof, or a homolog or a fragment thereof, and being depicted in the same respective line as said Ydr273w, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ydr273w, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ydr273w, or a functional equivalent or a homolog thereof as depicted in column 8 of Table II, application no. 12, preferably a homolog or functional equivalent as depicted in column 8 of Table II B, application no. 12, and being depicted in the same respective line as said Ydr273w, and preferably the activity is increased plastidic, whereby the respective line discloses in column 7 the fine chemical cerebronic acid.

Accordingly, in one embodiment, the molecule which activity is to be increased in the process of the invention is the gene product with an activity as a “ydr273w-protein”, preferably it is the molecule of section (a) or (b) of this paragraph.

In particular, it was observed that in plants, especially in Arabidopsis thaliana, increasing or generating the activity of a gene product plastidic with the activity of a “ydr273w-protein”, preferably being encoded by a gene comprising the nucleic acid sequence SEQ ID NO. 90159, preferably the coding region thereof, conferred the production of or the increase in cerebronic acid compared with the wild type control.

The nucleic acid sequence of Ydr273w from Saccharomyces cerevisiae, e.g. as shown in the respective line in column 5 of Table I, application no. 12, has been published in A. And the activity of the gene product thereof is the activity of ydr273w-protein.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “ydr273w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and be-ing depicted in the same respective line as said Ydr273w, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ydr273w, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ydr273w, or a functional equivalent or a homolog thereof as depicted in column 8 of Table II, application no. 12, preferably a homolog or functional equivalent as depicted in column 8 of Table II B, application no. 12, and being depicted in the same respective line as said Ydr273w, and preferably the activity is increased plastidic, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “ydr273w-protein”, preferably it is the molecule of section (a) or (b) of this paragraph.

In particular, it was observed that in plants, especially in Arabidopsis thaliana, increasing or generating the activity of a gene product plastidic with the activity of a “ydr273w-protein”, preferably being encoded by a gene comprising the nucleic acid sequence SEQ ID NO. 90159, preferably the coding region thereof, conferred the production of or the increase in 2-hydroxytetracosenoic acid (preferably 2-hydroxynervonic acid) compared with the wild type control.

The nucleic acid sequence of Ydr339c from Saccharomyces cerevisiae, e.g. as shown in the respective line in column 5 of Table I, application no. 12, has been published in A. And the activity of the gene product thereof is the activity of ydr339c-protein.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “ydr339c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said Ydr339c, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ydr339c, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ydr339c, or a functional equivalent or a homolog thereof as depicted in column 8 of Table II, application no. 12, preferably a homolog or functional equivalent as depicted in column 8 of Table II B, application no. 12, and being depicted in the same respective line as said Ydr339c, and preferably the activity is increased plastidic, whereby the respective line discloses in column 7 the fine chemical hexadecatrienoic acid.

Accordingly, in one embodiment, the molecule which activity is to be increased in the process of the invention is the gene product with an activity as a “ydr339c-protein”, preferably it is the molecule of section (a) or (b) of this paragraph.

In particular, it was observed that in plants, especially in Arabidopsis thaliana, increasing or generating the activity of a gene product plastidic with the activity of a “ydr339c-protein”, preferably being encoded by a gene comprising the nucleic acid sequence SEQ ID NO. 120241, preferably the coding region thereof, conferred the production of or the increase in hexadecatrienoic acid compared with the wild type control.

The nucleic acid sequence of Yel046c from Saccharomyces cerevisiae, e.g. as shown in the respective line in column 5 of Table I, application no. 12, has been published in A. And the activity of the gene product thereof is the activity of aldolase.

Accordingly, in one embodiment, the process of the present invention for producing heptadecanoic acid in a non-human organism, like a microorganism or a plant or a part thereof, comprises increasing or generating the activity of a gene product with the activity of a gene product conferring the activity of “aldolase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical heptadecanoic acid), application no. 12, preferably the coding region thereof, or a homolog or a fragment thereof, and being depicted in the same respective line as said Yel046c, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Yel046c, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Yel046c, or a functional equivalent or a homolog thereof as depicted in column 8 of Table II, application no. 12, preferably a homolog or functional equivalent as depicted in column 8 of Table II B, application no. 12, and being depicted in the same respective line as said Yel046c, and preferably the activity is increased plastidic, whereby the respective line discloses in column 7 the fine chemical heptadecanoic acid.

Accordingly, in one embodiment, the molecule which activity is to be increased in the process of the invention is the gene product with an activity as a “aldolase”, preferably it is the molecule of section (a) or (b) of this paragraph.

In particular, it was observed that in plants, especially in Arabidopsis thaliana, increasing or generating the activity of a gene product plastidic with the activity of a “aldolase”, preferably being encoded by a gene comprising the nucleic acid sequence SEQ ID NO. 128511, preferably the coding region thereof, conferred the production of or the increase in heptadecanoic acid compared with the wild type control.

The nucleic acid sequence of Yer112w from Saccharomyces cerevisiae, e.g. as shown in the respective line in column 5 of Table I, application no. 12, has been published in A. And the activity of the gene product thereof is the activity of small nuclear ribonucleoprotein .

Accordingly, in one embodiment, the process of the present invention for producing myristic acid in a non-human organism, like a microorganism or a plant or a part thereof, comprises increasing or generating the activity of a gene product with the activity of a gene product conferring the activity of “small nuclear ribonucleoprotein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical myristic acid), application no. 12, preferably the coding region thereof, or a homolog or a fragment thereof, and being depicted in the same respective line as said Yer112w, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Yer112w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Yer112w, or a functional equivalent or a homolog thereof as depicted in column 8 of Table II, application no. 12, preferably a homolog or functional equivalent as depicted in column 8 of Table II B, application no. 12, and being depicted in the same respective line as said Yer112w, and preferably the activity is increased non-targeted, whereby the respective line discloses in column 7 the fine chemical myristic acid.

Accordingly, in one embodiment, the molecule which activity is to be increased in the process of the invention is the gene product with an activity as a “small nuclear ribonucleoprotein”, preferably it is the molecule of section (a) or (b) of this paragraph.

In particular, it was observed that in plants, especially in Arabidopsis thaliana, increasing or generating the activity of a gene product non-targeted with the activity of a “small nuclear ribonucleoprotein”, preferably being encoded by a gene comprising the nucleic acid sequence SEQ ID NO. 113598, preferably the coding region thereof, conferred the production of or the increase in myristic acid compared with the wild type control.

The nucleic acid sequence of Yfl016c from Saccharomyces cerevisiae, e.g. as shown in the respective line in column 5 of Table I, application no. 12, has been published in A. And the activity of the gene product thereof is the activity of molecular chaperone portein.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “molecular chaperone portein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said Yfl016c, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Yfl016c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Yfl016c, or a functional equivalent or a homolog thereof as depicted in column 8 of Table II, application no. 12, preferably a homolog or functional equivalent as depicted in column 8 of Table II B, application no. 12, and being depicted in the same respective line as said Yfl016c, and preferably the activity is increased non-targeted, whereby the respective line discloses in column 7 the fine chemical hexadecadienoic acid.

Accordingly, in one embodiment, the molecule which activity is to be increased in the process of the invention is the gene product with an activity as a “molecular chaperone portein”, preferably it is the molecule of section (a) or (b) of this paragraph.

In particular, it was observed that in plants, especially in Arabidopsis thaliana, increasing or generating the activity of a gene product non-targeted with the activity of a “molecular chaperone portein”, preferably being encoded by a gene comprising the nucleic acid sequence SEQ ID NO. 63807, preferably the coding region thereof, conferred the production of or the increase in hexadecadienoic acid compared with the wild type control.

The nucleic acid sequence of Yfl016c from Saccharomyces cerevisiae, e.g. as shown in the respective line in column 5 of Table I, application no. 12, has been published in A. And the activity of the gene product thereof is the activity of molecular chaperone portein.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “molecular chaperone portein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said Yfl016c, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Yfl016c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Yfl016c, or a functional equivalent or a homolog thereof as depicted in column 8 of Table II, application no. 12, preferably a homolog or functional equivalent as depicted in column 8 of Table II B, application no. 12, and being depicted in the same respective line as said Yfl016c, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “molecular chaperone portein”, preferably it is the molecule of section (a) or (b) of this paragraph.

In particular, it was observed that in plants, especially in Arabidopsis thaliana, increasing or generating the activity of a gene product non-targeted with the activity of a “molecular chaperone portein”, preferably being encoded by a gene comprising the nucleic acid sequence SEQ ID NO. 63807, preferably the coding region thereof, conferred the production of or the increase in hexadecatrienoic acid compared with the wild type control.

The nucleic acid sequence of Yfl019c from Saccharomyces cerevisiae, e.g. as shown in the respective line in column 5 of Table I, application no. 12, has been published in A. And the activity of the gene product thereof is the activity of YFL019C-protein.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “YFL019C-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said Yfl019c, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Yfl019c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Yfl019c, or a functional equivalent or a homolog thereof as depicted in column 8 of Table II, application no. 12, preferably a homolog or functional equivalent as depicted in column 8 of Table II B, application no. 12, and being depicted in the same respective line as said Yfl019c, and preferably the activity is increased non-targeted, whereby the respective line discloses in column 7 the fine chemical 2-hydroxybehenic acid.

Accordingly, in one embodiment, the molecule which activity is to be increased in the process of the invention is the gene product with an activity as a “YFL019C-protein”, preferably it is the molecule of section (a) or (b) of this paragraph.

In particular, it was observed that in plants, especially in Arabidopsis thaliana, increasing or generating the activity of a gene product non-targeted with the activity of a “YFL019C-protein”, preferably being encoded by a gene comprising the nucleic acid sequence SEQ ID NO. 64144, preferably the coding region thereof, conferred the production of or the increase in 2-hydroxybehenic acid compared with the wild type control.

The nucleic acid sequence of Yfl019c from Saccharomyces cerevisiae, e.g. as shown in the respective line in column 5 of Table I, application no. 12, has been published in A. And the activity of the gene product thereof is the activity of YFL019C-protein.

Accordingly, in one embodiment, the process of the present invention for producing 2-hydroxypalmitic acid in a non-human organism, like a microorganism or a plant or a part thereof, comprises increasing or generating the activity of a gene product with the activity of a gene product conferring the activity of “YFL0190-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxypalmitic acid), application no. 12, preferably the coding region thereof, or a homolog or a fragment thereof, and being depicted in the same respective line as said Yfl019c, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Yfl019c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Yfl019c, or a functional equivalent or a homolog thereof as depicted in column 8 of Table II, application no. 12, preferably a homolog or functional equivalent as depicted in column 8 of Table II B, application no. 12, and being depicted in the same respective line as said Yfl019c, and preferably the activity is increased non-targeted, whereby the respective line discloses in column 7 the fine chemical 2-hydroxypalmitic acid.

Accordingly, in one embodiment, the molecule which activity is to be increased in the process of the invention is the gene product with an activity as a “YFL019C-protein”, preferably it is the molecule of section (a) or (b) of this paragraph.

In particular, it was observed that in plants, especially in Arabidopsis thaliana, increasing or generating the activity of a gene product non-targeted with the activity of a “YFL019C-protein”, preferably being encoded by a gene comprising the nucleic acid sequence SEQ ID NO. 64144, preferably the coding region thereof, conferred the production of or the increase in 2-hydroxypalmitic acid compared with the wild type control.

The nucleic acid sequence of Yfl030w from Saccharomyces cerevisiae, e.g. as shown in the respective line in column 5 of Table I, application no. 12, has been published in A. And the activity of the gene product thereof is the activity of aminotransferase.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “aminotransferase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said Yfl030w, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Yfl030w, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Yfl030w, or a functional equivalent or a homolog thereof as depicted in column 8 of Table II, application no. 12, preferably a homolog or functional equivalent as depicted in column 8 of Table II B, application no. 12, and being depicted in the same respective line as said Yfl030w, and preferably the activity is increased plastidic, whereby the respective line discloses in column 7 the fine chemical hexadecadienoic acid.

Accordingly, in one embodiment, the molecule which activity is to be increased in the process of the invention is the gene product with an activity as a “aminotransferase”, preferably it is the molecule of section (a) or (b) of this paragraph.

In particular, it was observed that in plants, especially in Arabidopsis thaliana, increasing or generating the activity of a gene product plastidic with the activity of a “aminotransferase”, preferably being encoded by a gene comprising the nucleic acid sequence SEQ ID NO. 90189, preferably the coding region thereof, conferred the production of or the increase in hexadecadienoic acid compared with the wild type control.

The nucleic acid sequence of Ygl237c from Saccharomyces cerevisiae, e.g. as shown in the respective line in column 5 of Table I, application no. 12, has been published in A. And the activity of the gene product thereof is the activity of ygl237c-protein.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “ygl237c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said Ygl237c, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ygl237c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ygl237c, or a functional equivalent or a homolog thereof as depicted in column 8 of Table II, application no. 12, preferably a homolog or functional equivalent as depicted in column 8 of Table II B, application no. 12, and being depicted in the same respective line as said Ygl237c, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “ygl237c-protein”, preferably it is the molecule of section (a) or (b) of this paragraph.

In particular, it was observed that in plants, especially in Arabidopsis thaliana, increasing or generating the activity of a gene product non-targeted with the activity of a “ygl237c-protein”, preferably being encoded by a gene comprising the nucleic acid sequence SEQ ID NO. 64177, preferably the coding region thereof, conferred the production of or the increase in hexadecatrienoic acid compared with the wild type control.

The nucleic acid sequence of Ygr104c from Saccharomyces cerevisiae, e.g. as shown in the respective line in column 5 of Table I, application no. 12, has been published in A. And the activity of the gene product thereof is the activity of YGR104C-protein.

Accordingly, in one embodiment, the process of the present invention for producing eicosanoic acid in a non-human organism, like a microorganism or a plant or a part thereof, comprises increasing or generating the activity of a gene product with the activity of a gene product conferring the activity of “YGR104C-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical eicosanoic acid), application no. 12, preferably the coding region thereof, or a homolog or a fragment thereof, and being depicted in the same respective line as said Ygr104c, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ygr104c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ygr104c, or a functional equivalent or a homolog thereof as depicted in column 8 of Table II, application no. 12, preferably a homolog or functional equivalent as depicted in column 8 of Table II B, application no. 12, and being depicted in the same respective line as said Ygr104c, and preferably the activity is increased non-targeted, whereby the respective line discloses in column 7 the fine chemical eicosanoic acid.

Accordingly, in one embodiment, the molecule which activity is to be increased in the process of the invention is the gene product with an activity as a “YGR104C-protein”, preferably it is the molecule of section (a) or (b) of this paragraph.

In particular, it was observed that in plants, especially in Arabidopsis thaliana, increasing or generating the activity of a gene product non-targeted with the activity of a “YGR104C-protein”, preferably being encoded by a gene comprising the nucleic acid sequence SEQ ID NO. 117482, preferably the coding region thereof, conferred the production of or the increase in eicosanoic acid compared with the wild type control.

The nucleic acid sequence of Ygr175c from Saccharomyces cerevisiae, e.g. as shown in the respective line in column 5 of Table I, application no. 12, has been published in A. And the activity of the gene product thereof is the activity of squalene monooxygenase.

Accordingly, in one embodiment, the process of the present invention for producing cerebronic acid in a non-human organism, like a microorganism or a plant or a part thereof, comprises increasing or generating the activity of a gene product with the activity of a gene product conferring the activity of “squalene monooxygenase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical cerebronic acid), application no. 12, preferably the coding region thereof, or a homolog or a fragment thereof, and being depicted in the same respective line as said Ygr175c, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ygr175c, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ygr175c, or a functional equivalent or a homolog thereof as depicted in column 8 of Table II, application no. 12, preferably a homolog or functional equivalent as depicted in column 8 of Table II B, application no. 12, and being depicted in the same respective line as said Ygr175c, and preferably the activity is increased plastidic, whereby the respective line discloses in column 7 the fine chemical cerebronic acid.

Accordingly, in one embodiment, the molecule which activity is to be increased in the process of the invention is the gene product with an activity as a “squalene monooxygenase”, preferably it is the molecule of section (a) or (b) of this paragraph.

In particular, it was observed that in plants, especially in Arabidopsis thaliana, increasing or generating the activity of a gene product plastidic with the activity of a “squalene monooxygenase”, preferably being encoded by a gene comprising the nucleic acid sequence SEQ ID NO. 64218, preferably the coding region thereof, conferred the production of or the increase in cerebronic acid compared with the wild type control.

The nucleic acid sequence of Ygr286c from Saccharomyces cerevisiae, e.g. as shown in the respective line in column 5 of Table I, application no. 12, has been published in A. And the activity of the gene product thereof is the activity of biotin synthase.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “biotin synthase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said Ygr286c, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ygr286c, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ygr286c, or a functional equivalent or a homolog thereof as depicted in column 8 of Table II, application no. 12, preferably a homolog or functional equivalent as depicted in column 8 of Table II B, application no. 12, and being depicted in the same respective line as said Ygr286c, and preferably the activity is increased plastidic, whereby the respective line discloses in column 7 the fine chemical hexadecadienoic acid.

Accordingly, in one embodiment, the molecule which activity is to be increased in the process of the invention is the gene product with an activity as a “biotin synthase”, preferably it is the molecule of section (a) or (b) of this paragraph.

In particular, it was observed that in plants, especially in Arabidopsis thaliana, increasing or generating the activity of a gene product plastidic with the activity of a “biotin synthase”, preferably being encoded by a gene comprising the nucleic acid sequence SEQ ID NO. 128655, preferably the coding region thereof, conferred the production of or the increase in hexadecadienoic acid compared with the wild type control.

The nucleic acid sequence of Yhr033w from Saccharomyces cerevisiae, e.g. as shown in the respective line in column 5 of Table I, application no. 12, has been published in A. And the activity of the gene product thereof is the activity of yhr033w-protein.

Accordingly, in one embodiment, the process of the present invention for producing cerebronic acid in a non-human organism, like a microorganism or a plant or a part thereof, comprises increasing or generating the activity of a gene product with the activity of a gene product conferring the activity of “yhr033w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical cerebronic acid), application no. 12, preferably the coding region thereof, or a homolog or a fragment thereof, and being depicted in the same respective line as said Yhr033w, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Yhr033w, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Yhr033w, or a functional equivalent or a homolog thereof as depicted in column 8 of Table II, application no. 12, preferably a homolog or functional equivalent as depicted in column 8 of Table II B, application no. 12, and being depicted in the same respective line as said Yhr033w, and preferably the activity is increased plastidic, whereby the respective line discloses in column 7 the fine chemical cerebronic acid.

Accordingly, in one embodiment, the molecule which activity is to be increased in the process of the invention is the gene product with an activity as a “yhr033w-protein”, preferably it is the molecule of section (a) or (b) of this paragraph.

In particular, it was observed that in plants, especially in Arabidopsis thaliana, increasing or generating the activity of a gene product plastidic with the activity of a “yhr033w-protein”, preferably being encoded by a gene comprising the nucleic acid sequence SEQ ID NO. 128967, preferably the coding region thereof, conferred the production of or the increase in cerebronic acid compared with the wild type control.

The nucleic acid sequence of Yjl073w from Saccharomyces cerevisiae, e.g. as shown in the respective line in column 5 of Table I, application no. 12, has been published in A. And the activity of the gene product thereof is the activity of DnaJ-like chaperone.

Accordingly, in one embodiment, the process of the present invention for producing cerebronic acid in a non-human organism, like a microorganism or a plant or a part thereof, comprises increasing or generating the activity of a gene product with the activity of a gene product conferring the activity of “DnaJ-like chaperone”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical cerebronic acid), application no. 12, preferably the coding region thereof, or a homolog or a fragment thereof, and being depicted in the same respective line as said Yjl073w, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Yjl073w, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Yjl073w, or a functional equivalent or a homolog thereof as depicted in column 8 of Table II, application no. 12, preferably a homolog or functional equivalent as depicted in column 8 of Table II B, application no. 12, and being depicted in the same respective line as said Yjl073w, and preferably the activity is increased plastidic, whereby the respective line discloses in column 7 the fine chemical cerebronic acid.

Accordingly, in one embodiment, the molecule which activity is to be increased in the process of the invention is the gene product with an activity as a “DnaJ-like chaperone”, preferably it is the molecule of section (a) or (b) of this paragraph.

In particular, it was observed that in plants, especially in Arabidopsis thaliana, increasing or generating the activity of a gene product plastidic with the activity of a “DnaJ-like chaperone”, preferably being encoded by a gene comprising the nucleic acid sequence SEQ ID NO. 64964, preferably the coding region thereof, conferred the production of or the increase in cerebronic acid compared with the wild type control.

The nucleic acid sequence of Yjl127w-a from Saccharomyces cerevisiae, e.g. as shown in the respective line in column 5 of Table I, application no. 12, has been published in A. And the activity of the gene product thereof is the activity of YJL127W-A-protein.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “YJL127W-A-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said Yjl127w-a, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Yjl127w-a, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Yjl127w-a, or a functional equivalent or a homolog thereof as depicted in column 8 of Table II, application no. 12, preferably a homolog or functional equivalent as depicted in column 8 of Table II B, application no. 12, and being depicted in the same respective line as said Yjl127w-a, and preferably the activity is increased non-targeted, whereby the respective line discloses in column 7 the fine chemical 2-hydroxybehenic acid.

Accordingly, in one embodiment, the molecule which activity is to be increased in the process of the invention is the gene product with an activity as a “YJL127W-A-protein”, preferably it is the molecule of section (a) or (b) of this paragraph.

In particular, it was observed that in plants, especially in Arabidopsis thaliana, increasing or generating the activity of a gene product non-targeted with the activity of a “YJL127W-A-protein”, preferably being encoded by a gene comprising the nucleic acid sequence SEQ ID NO. 97631, preferably the coding region thereof, conferred the production of or the increase in 2-hydroxybehenic acid compared with the wild type control.

The nucleic acid sequence of Yjr130c from Saccharomyces cerevisiae, e.g. as shown in the respective line in column 5 of Table I, application no. 12, has been published in A. And the activity of the gene product thereof is the activity of cystathionine gamma-synthase.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “cystathionine gamma-synthase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said Yjr130c, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Yjr130c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Yjr130c, or a functional equivalent or a homolog thereof as depicted in column 8 of Table II, application no. 12, preferably a homolog or functional equivalent as depicted in column 8 of Table II B, application no. 12, and being depicted in the same respective line as said Yjr130c, and preferably the activity is increased non-targeted, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “cystathionine gamma-synthase”, preferably it is the molecule of section (a) or (b) of this paragraph.

In particular, it was observed that in plants, especially in Arabidopsis thaliana, increasing or generating the activity of a gene product non-targeted with the activity of a “cystathionine gamma-synthase”, preferably being encoded by a gene comprising the nucleic acid sequence SEQ ID NO. 14843, preferably the coding region thereof, conferred the production of or the increase in 2-hydroxytetracosenoic acid (preferably 2-hydroxynervonic acid) compared with the wild type control.

The nucleic acid sequence of Yjr130c from Saccharomyces cerevisiae, e.g. as shown in the respective line in column 5 of Table I, application no. 12, has been published in A. And the activity of the gene product thereof is the activity of cystathionine gamma-synthase.

Accordingly, in one embodiment, the process of the present invention for producing 2-hydroxypalmitic acid in a non-human organism, like a microorganism or a plant or a part thereof, comprises increasing or generating the activity of a gene product with the activity of a gene product conferring the activity of “cystathionine gamma-synthase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxypalmitic acid), application no. 12, preferably the coding region thereof, or a homolog or a fragment thereof, and being depicted in the same respective line as said Yjr130c, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Yjr130c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Yjr130c, or a functional equivalent or a homolog thereof as depicted in column 8 of Table II, application no. 12, preferably a homolog or functional equivalent as depicted in column 8 of Table II B, application no. 12, and being depicted in the same respective line as said Yjr130c, and preferably the activity is increased non-targeted, whereby the respective line discloses in column 7 the fine chemical 2-hydroxypalmitic acid.

Accordingly, in one embodiment, the molecule which activity is to be increased in the process of the invention is the gene product with an activity as a “cystathionine gamma-synthase”, preferably it is the molecule of section (a) or (b) of this paragraph.

In particular, it was observed that in plants, especially in Arabidopsis thaliana, increasing or generating the activity of a gene product non-targeted with the activity of a “cystathionine gamma-synthase”, preferably being encoded by a gene comprising the nucleic acid sequence SEQ ID NO. 14843, preferably the coding region thereof, conferred the production of or the increase in 2-hydroxypalmitic acid compared with the wild type control.

The nucleic acid sequence of Yjr153w from Saccharomyces cerevisiae, e.g. as shown in the respective line in column 5 of Table I, application no. 12, has been published in A. And the activity of the gene product thereof is the activity of polygalacturonase.

Accordingly, in one embodiment, the process of the present invention for producing 2-hydroxypalmitic acid in a non-human organism, like a microorganism or a plant or a part thereof, comprises increasing or generating the activity of a gene product with the activity of a gene product conferring the activity of “polygalacturonase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxypalmitic acid), application no. 12, preferably the coding region thereof, or a homolog or a fragment thereof, and being depicted in the same respective line as said Yjr153w, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Yjr153w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Yjr153w, or a functional equivalent or a homolog thereof as depicted in column 8 of Table II, application no. 12, preferably a homolog or functional equivalent as depicted in column 8 of Table II B, application no. 12, and being depicted in the same respective line as said Yjr153w, and preferably the activity is increased non-targeted, whereby the respective line discloses in column 7 the fine chemical 2-hydroxypalmitic acid.

Accordingly, in one embodiment, the molecule which activity is to be increased in the process of the invention is the gene product with an activity as a “polygalacturonase”, preferably it is the molecule of section (a) or (b) of this paragraph.

In particular, it was observed that in plants, especially in Arabidopsis thaliana, increasing or generating the activity of a gene product non-targeted with the activity of a “polygalacturonase”, preferably being encoded by a gene comprising the nucleic acid sequence SEQ ID NO. 66274, preferably the coding region thereof, conferred the production of or the increase in 2-hydroxypalmitic acid compared with the wild type control.

The nucleic acid sequence of Ylr210w from Saccharomyces cerevisiae, e.g. as shown in the respective line in column 5 of Table I, application no. 12, has been published in A. And the activity of the gene product thereof is the activity of cyclin.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “cyclin”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said Ylr210w, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ylr210w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ylr210w, or a functional equivalent or a homolog thereof as depicted in column 8 of Table II, application no. 12, preferably a homolog or functional equivalent as depicted in column 8 of Table II B, application no. 12, and being depicted in the same respective line as said Ylr210w, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “cyclin”, preferably it is the molecule of section (a) or (b) of this paragraph.

In particular, it was observed that in plants, especially in Arabidopsis thaliana, increasing or generating the activity of a gene product non-targeted with the activity of a “cyclin”, preferably being encoded by a gene comprising the nucleic acid sequence SEQ ID NO. 129065, preferably the coding region thereof, conferred the production of or the increase in hexadecatrienoic acid compared with the wild type control.

The nucleic acid sequence of Ylr326w from Saccharomyces cerevisiae, e.g. as shown in the respective line in column 5 of Table I, application no. 12, has been published in A. And the activity of the gene product thereof is the activity of ylr326w-protein.

Accordingly, in one embodiment, the process of the present invention for producing hexadecadienoic acid in a non-human organism, like a microorganism or a plant or a part thereof, comprises increasing or generating the activity of a gene product with the activity of a gene product conferring the activity of “ylr326w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecadienoic acid), application no. 12, preferably the coding region thereof, or a homolog or a fragment thereof, and being depicted in the same respective line as said Ylr326w, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ylr326w, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ylr326w, or a functional equivalent or a homolog thereof as depicted in column 8 of Table II, application no. 12, preferably a homolog or functional equivalent as depicted in column 8 of Table II B, application no. 12, and being depicted in the same respective line as said Ylr326w, and preferably the activity is increased plastidic, whereby the respective line discloses in column 7 the fine chemical hexadecadienoic acid.

Accordingly, in one embodiment, the molecule which activity is to be increased in the process of the invention is the gene product with an activity as a “ylr326w-protein”, preferably it is the molecule of section (a) or (b) of this paragraph.

In particular, it was observed that in plants, especially in Arabidopsis thaliana, increasing or generating the activity of a gene product plastidic with the activity of a “ylr326w-protein”, preferably being encoded by a gene comprising the nucleic acid sequence SEQ ID NO. 129123, preferably the coding region thereof, conferred the production of or the increase in hexadecadienoic acid compared with the wild type control.

The nucleic acid sequence of Ylr326w from Saccharomyces cerevisiae, e.g. as shown in the respective line in column 5 of Table I, application no. 12, has been published in A. And the activity of the gene product thereof is the activity of ylr326w-protein.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “ylr326w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said Ylr326w, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ylr326w, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ylr326w, or a functional equivalent or a homolog thereof as depicted in column 8 of Table II, application no. 12, preferably a homolog or functional equivalent as depicted in column 8 of Table II B, application no. 12, and being depicted in the same respective line as said Ylr326w, and preferably the activity is increased plastidic, whereby the respective line discloses in column 7 the fine chemical hexadecatrienoic acid.

Accordingly, in one embodiment, the molecule which activity is to be increased in the process of the invention is the gene product with an activity as a “ylr326w-protein”, preferably it is the molecule of section (a) or (b) of this paragraph.

In particular, it was observed that in plants, especially in Arabidopsis thaliana, increasing or generating the activity of a gene product plastidic with the activity of a “ylr326w-protein”, preferably being encoded by a gene comprising the nucleic acid sequence SEQ ID NO. 129123, preferably the coding region thereof, conferred the production of or the increase in hexadecatrienoic acid compared with the wild type control.

The nucleic acid sequence of Yml083c from Saccharomyces cerevisiae, e.g. as shown in the respective line in column 5 of Table I, application no. 12, has been published in A. And the activity of the gene product thereof is the activity of yml083c-protein.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “yml083c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said Yml083c, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Yml083c, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Yml083c, or a functional equivalent or a homolog thereof as depicted in column 8 of Table II, application no. 12, preferably a homolog or functional equivalent as depicted in column 8 of Table II B, application no. 12, and being depicted in the same respective line as said Yml083c, and preferably the activity is increased plastidic, whereby the respective line discloses in column 7 the fine chemical 2-hydroxybehenic acid.

Accordingly, in one embodiment, the molecule which activity is to be increased in the process of the invention is the gene product with an activity as a “yml083c-protein”, preferably it is the molecule of section (a) or (b) of this paragraph.

In particular, it was observed that in plants, especially in Arabidopsis thaliana, increasing or generating the activity of a gene product plastidic with the activity of a “yml083c-protein”, preferably being encoded by a gene comprising the nucleic acid sequence SEQ ID NO. 91781, preferably the coding region thereof, conferred the production of or the increase in 2-hydroxybehenic acid compared with the wild type control.

The nucleic acid sequence of Yml084w from Saccharomyces cerevisiae, e.g. as shown in the respective line in column 5 of Table I, application no. 12, has been published in A. And the activity of the gene product thereof is the activity of yml084w-protein.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “yml084w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said Yml084w, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Yml084w, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Yml084w, or a functional equivalent or a homolog thereof as depicted in column 8 of Table II, application no. 12, preferably a homolog or functional equivalent as depicted in column 8 of Table II B, application no. 12, and being depicted in the same respective line as said Yml084w, and preferably the activity is increased plastidic, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “yml084w-protein”, preferably it is the molecule of section (a) or (b) of this paragraph.

In particular, it was observed that in plants, especially in Arabidopsis thaliana, increasing or generating the activity of a gene product plastidic with the activity of a “yml084w-protein”, preferably being encoded by a gene comprising the nucleic acid sequence SEQ ID NO. 15175, preferably the coding region thereof, conferred the production of or the increase in 2-hydroxytetracosenoic acid (preferably 2-hydroxynervonic acid) compared with the wild type control.

The nucleic acid sequence of Ymr186w from Saccharomyces cerevisiae, e.g. as shown in the respective line in column 5 of Table I, application no. 12, has been published in A. And the activity of the gene product thereof is the activity of molecular chaperone.

Accordingly, in one embodiment, the process of the present invention for producing 2-hydroxypalmitic acid in a non-human organism, like a microorganism or a plant or a part thereof, comprises increasing or generating the activity of a gene product with the activity of a gene product conferring the activity of “molecular chaperone”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxypalmitic acid), application no. 12, preferably the coding region thereof, or a homolog or a fragment thereof, and being depicted in the same respective line as said Ymr186w, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ymr186w, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ymr186w, or a functional equivalent or a homolog thereof as depicted in column 8 of Table II, application no. 12, preferably a homolog or functional equivalent as depicted in column 8 of Table II B, application no. 12, and being depicted in the same respective line as said Ymr186w, and preferably the activity is increased plastidic, whereby the respective line discloses in column 7 the fine chemical 2-hydroxypalmitic acid.

Accordingly, in one embodiment, the molecule which activity is to be increased in the process of the invention is the gene product with an activity as a “molecular chaperone”, preferably it is the molecule of section (a) or (b) of this paragraph.

In particular, it was observed that in plants, especially in Arabidopsis thaliana, increasing or generating the activity of a gene product plastidic with the activity of a “molecular chaperone”, preferably being encoded by a gene comprising the nucleic acid sequence SEQ ID NO. 129141, preferably the coding region thereof, conferred the production of or the increase in 2-hydroxypalmitic acid compared with the wild type control.

The nucleic acid sequence of Ynl130c from Saccharomyces cerevisiae, e.g. as shown in the respective line in column 5 of Table I, application no. 12, has been published in A. And the activity of the gene product thereof is the activity of phosphotransferase.

Accordingly, in one embodiment, the process of the present invention for producing oleic acid in a non-human organism, like a microorganism or a plant or a part thereof, comprises increasing or generating the activity of a gene product with the activity of a gene product conferring the activity of “phosphotransferase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical oleic acid), application no. 12, preferably the coding region thereof, or a homolog or a fragment thereof, and being depicted in the same respective line as said Ynl130c, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ynl130c, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ynl130c, or a functional equivalent or a homolog thereof as depicted in column 8 of Table II, application no. 12, preferably a homolog or functional equivalent as depicted in column 8 of Table II B, application no. 12, and being depicted in the same respective line as said Ynl130c, and preferably the activity is increased mitochondrial, whereby the respective line discloses in column 7 the fine chemical oleic acid.

Accordingly, in one embodiment, the molecule which activity is to be increased in the process of the invention is the gene product with an activity as a “phosphotransferase”, preferably it is the molecule of section (a) or (b) of this paragraph.

In particular, it was observed that in plants, especially in Arabidopsis thaliana, increasing or generating the activity of a gene product mitochondrial with the activity of a “phosphotransferase”, preferably being encoded by a gene comprising the nucleic acid sequence SEQ ID NO. 129470, preferably the coding region thereof, conferred the production of or the increase in oleic acid compared with the wild type control.

The nucleic acid sequence of Ynl321w from Saccharomyces cerevisiae, e.g. as shown in the respective line in column 5 of Table I, application no. 12, has been published in A. And the activity of the gene product thereof is the activity of ynl321w-protein.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “ynl321w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said Ynl321w, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ynl321w, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ynl321w, or a functional equivalent or a homolog thereof as depicted in column 8 of Table II, application no. 12, preferably a homolog or functional equivalent as depicted in column 8 of Table II B, application no. 12, and being depicted in the same respective line as said Ynl321w, and preferably the activity is increased plastidic, whereby the respective line discloses in column 7 the fine chemical 2-hydroxybehenic acid.

Accordingly, in one embodiment, the molecule which activity is to be increased in the process of the invention is the gene product with an activity as a “ynl321w-protein”, preferably it is the molecule of section (a) or (b) of this paragraph.

In particular, it was observed that in plants, especially in Arabidopsis thaliana, increasing or generating the activity of a gene product plastidic with the activity of a “ynl321w-protein”, preferably being encoded by a gene comprising the nucleic acid sequence SEQ ID NO. 129546, preferably the coding region thereof, conferred the production of or the increase in 2-hydroxybehenic acid compared with the wild type control.

The nucleic acid sequence of Yor187w from Saccharomyces cerevisiae, e.g. as shown in the respective line in column 5 of Table I, application no. 12, has been published in A. And the activity of the gene product thereof is the activity of Elongation factor Tu.

Accordingly, in one embodiment, the process of the present invention for producing hexadecatrienoic acid in a non-human organism, like a microorganism or a plant or a part thereof, comprises increasing or generating the activity of a gene product with the activity of a gene product conferring the activity of “Elongation factor Tu”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical hexadecatrienoic acid), application no. 12, preferably the coding region thereof, or a homolog or a fragment thereof, and being depicted in the same respective line as said Yor187w, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Yor187w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Yor187w, or a functional equivalent or a homolog thereof as depicted in column 8 of Table II, application no. 12, preferably a homolog or functional equivalent as depicted in column 8 of Table II B, application no. 12, and being depicted in the same respective line as said Yor187w, and preferably the activity is increased non-targeted, whereby the respective line discloses in column 7 the fine chemical hexadecatrienoic acid.

Accordingly, in one embodiment, the molecule which activity is to be increased in the process of the invention is the gene product with an activity as a “Elongation factor Tu”, preferably it is the molecule of section (a) or (b) of this paragraph.

In particular, it was observed that in plants, especially in Arabidopsis thaliana, increasing or generating the activity of a gene product non-targeted with the activity of a “Elongation factor Tu”, preferably being encoded by a gene comprising the nucleic acid sequence SEQ ID NO. 129574, preferably the coding region thereof, conferred the production of or the increase in hexadecatrienoic acid compared with the wild type control.

The nucleic acid sequence of Ypl271w from Saccharomyces cerevisiae, e.g. as shown in the respective line in column 5 of Table I, application no. 12, has been published in A. And the activity of the gene product thereof is the activity of ATPase epsilon subunit.

Accordingly, in one embodiment, the process of the present invention for producing 2-hydroxytetracosenoic acid (preferably 2-hydroxynervonic acid) in a non-human organism, like a microorganism or a plant or a part thereof, comprises increasing or generating the activity of a gene product with the activity of a gene product conferring the activity of “ATPase epsilon subunit”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxytetracosenoic acid (preferably 2-hydroxynervonic acid)), application no. 12, preferably the coding region thereof, or a homolog or a fragment thereof, and being depicted in the same respective line as said Ypl271w, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ypl271w, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ypl271w, or a functional equivalent or a homolog thereof as depicted in column 8 of Table II, application no. 12, preferably a homolog or functional equivalent as depicted in column 8 of Table II B, application no. 12, and being depicted in the same respective line as said Ypl271w, and preferably the activity is increased plastidic, whereby the respective line discloses in column 7 the fine chemical 2-hydroxytetracosenoic acid (preferably 2-hydroxynervonic acid).

Accordingly, in one embodiment, the molecule which activity is to be increased in the process of the invention is the gene product with an activity as a “ATPase epsilon subunit”, preferably it is the molecule of section (a) or (b) of this paragraph.

In particular, it was observed that in plants, especially in Arabidopsis thaliana, increasing or generating the activity of a gene product plastidic with the activity of a “ATPase epsilon subunit”, preferably being encoded by a gene comprising the nucleic acid sequence SEQ ID NO. 118212, preferably the coding region thereof, conferred the production of or the increase in 2-hydroxytetracosenoic acid (preferably 2-hydroxynervonic acid) compared with the wild type control.

The nucleic acid sequence of Ypr167c from Saccharomyces cerevisiae, e.g. as shown in the respective line in column 5 of Table I, application no. 12, has been published in A. And the activity of the gene product thereof is the activity of phosphoadenosine phosphosulfate reductase.

Accordingly, in one embodiment, the process of the present invention for producing 2-hydroxypalmitic acid in a non-human organism, like a microorganism or a plant or a part thereof, comprises increasing or generating the activity of a gene product with the activity of a gene product conferring the activity of “phosphoadenosine phosphosulfate reductase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxypalmitic acid), application no. 12, preferably the coding region thereof, or a homolog or a fragment thereof, and being depicted in the same respective line as said Ypr167c, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said Ypr167c, and preferably the activity is increased plastidic, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said Ypr167c, or a functional equivalent or a homolog thereof as depicted in column 8 of Table II, application no. 12, preferably a homolog or functional equivalent as depicted in column 8 of Table II B, application no. 12, and being depicted in the same respective line as said Ypr167c, and preferably the activity is increased plastidic, whereby the respective line discloses in column 7 the fine chemical 2-hydroxypalmitic acid.

Accordingly, in one embodiment, the molecule which activity is to be increased in the process of the invention is the gene product with an activity as a “phosphoadenosine phosphosulfate reductase”, preferably it is the molecule of section (a) or (b) of this paragraph.

In particular, it was observed that in plants, especially in Arabidopsis thaliana, increasing or generating the activity of a gene product plastidic with the activity of a “phosphoadenosine phosphosulfate reductase”, preferably being encoded by a gene comprising the nucleic acid sequence SEQ ID NO. 130181, preferably the coding region thereof, conferred the production of or the increase in 2-hydroxypalmitic acid compared with the wild type control.

The nucleic acid sequence of YDR447C from Saccharomyces cerevisiae, e.g. as shown in the respective line in column 5 of Table I, application no. 12, has been published in A. And the activity of the gene product thereof is the activity of 40S ribosomal protein S17.

Accordingly, in one embodiment, the process of the present invention for producing oleic acid in a non-human organism, like a microorganism or a plant or a part thereof, comprises increasing or generating the activity of a gene product with the activity of a gene product conferring the activity of “40S ribosomal protein S17”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line discloses in column 7 the fine chemical oleic acid), application no. 12, preferably the coding region thereof, or a homolog or a fragment thereof, and being depicted in the same respective line as said YDR447C, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said YDR447C, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said YDR447C, or a functional equivalent or a homolog thereof as depicted in column 8 of Table II, application no. 12, preferably a homolog or functional equivalent as depicted in column 8 of Table II B, application no. 12, and being depicted in the same respective line as said YDR447C, and preferably the activity is increased non-targeted, whereby the respective line discloses in column 7 the fine chemical oleic acid.

Accordingly, in one embodiment, the molecule which activity is to be increased in the process of the invention is the gene product with an activity as a “405 ribosomal protein S17”, preferably it is the molecule of section (a) or (b) of this paragraph.

In particular, it was observed that in plants, especially in Arabidopsis thaliana, increasing or generating the activity of a gene product non-targeted with the activity of a “40S ribosomal protein S17”, preferably being encoded by a gene comprising the nucleic acid sequence SEQ ID NO. 128161, preferably the coding region thereof, conferred the production of or the increase in oleic acid compared with the wild type control.

The nucleic acid sequence of B3644 from Escherichia coli, e.g. as shown in the respective line in column 5 of Table I, application no. 12, has been published in B. And the activity of the gene product thereof is the activity of b3644-protein.

Accordingly, in one embodiment, the process of the present invention for producing 2-hydroxybehenic acid in a non-human organism, like a microorganism or a plant or a part thereof, comprises increasing or generating the activity of a gene product with the activity of a gene product conferring the activity of “b3644-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical 2-hydroxybehenic acid), application no. 12, preferably the coding region thereof, or a homolog or a fragment thereof, and being depicted in the same respective line as said B3644, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B3644, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B3644, or a functional equivalent or a homolog thereof as depicted in column 8 of Table II, application no. 12, preferably a homolog or functional equivalent as depicted in column 8 of Table II B, application no. 12, and being depicted in the same respective line as said B3644, and preferably the activity is increased non-targeted, whereby the respective line discloses in column 7 the fine chemical 2-hydroxybehenic acid.

Accordingly, in one embodiment, the molecule which activity is to be increased in the process of the invention is the gene product with an activity as a “b3644-protein”, preferably it is the molecule of section (a) or (b) of this paragraph.

In particular, it was observed that in plants, especially in Arabidopsis thaliana, increasing or generating the activity of a gene product non-targeted with the activity of a “b3644-protein”, preferably being encoded by a gene comprising the nucleic acid sequence SEQ ID NO. 125348, preferably the coding region thereof, conferred the production of or the increase in 2-hydroxybehenic acid compared with the wild type control.

The nucleic acid sequence of B3644 from Escherichia coli, e.g. as shown in the respective line in column 5 of Table I, application no. 12, has been published in B. And the activity of the gene product thereof is the activity of b3644-protein.

Accordingly, in one embodiment, the process of the present invention for producing behenic acid in a non-human organism, like a microorganism or a plant or a part thereof, comprises increasing or generating the activity of a gene product with the activity of a gene product conferring the activity of “b3644-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical behenic acid), application no. 12, preferably the coding region thereof, or a homolog or a fragment thereof, and being depicted in the same respective line as said B3644, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B3644, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B3644, or a functional equivalent or a homolog thereof as depicted in column 8 of Table II, application no. 12, preferably a homolog or functional equivalent as depicted in column 8 of Table II B, application no. 12, and being depicted in the same respective line as said B3644, and preferably the activity is increased non-targeted, whereby the respective line discloses in column 7 the fine chemical behenic acid.

Accordingly, in one embodiment, the molecule which activity is to be increased in the process of the invention is the gene product with an activity as a “b3644-protein”, preferably it is the molecule of section (a) or (b) of this paragraph.

In particular, it was observed that in plants, especially in Arabidopsis thaliana, increasing or generating the activity of a gene product non-targeted with the activity of a “b3644-protein”, preferably being encoded by a gene comprising the nucleic acid sequence SEQ ID NO. 125348, preferably the coding region thereof, conferred the production of or the increase in behenic acid compared with the wild type control.

The nucleic acid sequence of B3644 from Escherichia coli, e.g. as shown in the respective line in column 5 of Table I, application no. 12, has been published in B. And the activity of the gene product thereof is the activity of b3644-protein.

Accordingly, in one embodiment, the process of the present invention for producing nervonic acid in a non-human organism, like a microorganism or a plant or a part thereof, comprises increasing or generating the activity of a gene product with the activity of a gene product conferring the activity of “b3644-protein”, especially from Escherichia coli or its functional equivalent or its homolog, e.g. the increase of
(a) a gene product of a gene comprising the nucleic acid molecule as shown in the respective line in column 5 of Table I (whereby the respective line discloses in column 7 the fine chemical nervonic acid), application no. 12, preferably the coding region thereof, or a homolog or a fragment thereof, and being depicted in the same respective line as said B3644, or a functional equivalent or a homolog thereof as shown in column 8 of Table I, application no. 12, preferably the coding region thereof, particularly a homolog or functional equivalent as shown in column 8 of Table I B, application no. 12, and being depicted in the same respective line as said B3644, and preferably the activity is increased non-targeted, or
(b) a polypeptide comprising a polypeptide, a consensus sequence or at least a polypeptide motif as shown in the respective line in column 5 of Table II or in column 8 of Table IV, application no. 12, respectively, and being depicted in the same respective line as said B3644, or a functional equivalent or a homolog thereof as depicted in column 8 of Table II, application no. 12, preferably a homolog or functional equivalent as depicted in column 8 of Table II B, application no. 12, and being depicted in the same respective line as said B3644, and preferably the activity is increased non-targeted, whereby the respective line discloses in column 7 the fine chemical nervonic acid.

Accordingly, in one embodiment, the molecule which activity is to be increased in the process of the invention is the gene product with an activity as a “b3644-protein”, preferably it is the molecule of section (a) or (b) of this paragraph.

In particular, it was observed that in plants, especially in Arabidopsis thaliana, increasing or generating the activity of a gene product non-targeted with the activity of a “b3644-protein”, preferably being encoded by a gene comprising the nucleic acid sequence SEQ ID NO. 125348, preferably the coding region thereof, conferred the production of or the increase in nervonic acid compared with the wild type control.

[0105.1.8.12] to [0107.1.8.12] for the disclosure of these paragraphs see [0105.1.8.8] to [0107.1.8.12] above.

A protein having an activity conferring an increase in the amount or level of the fine chemical 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid, upon targeting to the plastids or mitochondria or upon non-targeting, preferably has the structure of the respective polypeptide described herein, in particular of the polypeptides comprising the consensus sequence or at least one polypeptide motifs as shown in the respective line in Table IV, application no. 12, column 8 or of the polypeptide comprising an amino acid sequence as disclosed in the respective line in Table II, application no. 12, columns 5 or 8, or homologs or fragments thereof as described herein, or is encoded by the nucleic acid molecule characterized herein or the nucleic acid molecule according to the invention, for example by the nucleic acid molecule as shown in the respective line in Table I, application no. 12, columns 5 or 8, preferably the coding region thereof, or homologs or fragments thereof and has the herein mentioned activity.

[0109.1.8.12] to [0110.1.8.12] for the disclosure of these paragraphs see [0109.1.8.8] to [0110.1.8.8] above.

In one embodiment, the process of the present invention comprises one or more of the following steps
(a) stabilizing a protein conferring the generation or increased expression of a protein encoded by the nucleic acid molecule of the invention or of the polypeptide of the invention, e.g. of a polypeptide having the activity selected from the group consisting of 2,3-dihydroxyphenylpropionate 1,2-dioxygenase, 2-C-methyl-D-erythritol 4-phosphate cytidylyltransferase, 30S ribosomal protein S3, 3-ketoacyl-CoA thiolase, 3-oxoacyl-[acyl-carrier-protein] reductase, 3-oxoacyl-[acyl-carrier-protein] synthase, 40S ribosomal protein S17, ABC transporter component, ABC transporter permease protein, acetyl-coenzyme A synthetase, acid shock protein, acyl-CoA synthase, acyl-desaturase, adenosine kinase, aldolase, alkaline phosphatase isozyme conversion protein, amino acid ABC transporter permease protein, aminotransferase, ankyrin repeat protein, At2g33510-protein, ATPase epsilon subunit, ATP-binding component of a transport system, b0050-protein, b0249-protein, b0371-protein, b0518-protein, b0801-protein, b1046-protein, b1346-protein, b1470-protein, b1522-protein, b1670-protein, b1703-protein, b1837-protein, b2032-protein, b2099-protein, b2107-protein, b2126-protein, b2139-protein, b2360-protein, b2474-protein, b2513-protein, b2654-protein, b3442-protein, b3644-protein, b3812-protein, b3944-protein, b4023-protein, b4205-protein, beta-hydroxylase, betaine aldehyde dehydrogenase, bifunctional protein (phosphoribosyltransferase and regulatory protein), bifunctional purine biosynthesis protein, binding-protein-dependent transport systems inner membrane component, biotin synthase, calcium-dependent protein kinase, carbon dioxide concentrating mechanism protein, cardiolipin synthase, CDP-diacylglycerol-serine O-phosphatidyltransferase, cell division control protein, cell wall protein, chlorophyllase, citrate synthase, coproporphyrinogen III oxidase, cyclin, cystathionine gamma-synthase, cysteine synthase A, dihydrolipoamide dehydrogenase, DNA mismatch repair protein, DnaJ-like chaperone, electron transport complex protein, Elongation factor Tu, ethanolamine utilization protein, F-box protein, ferredoxin, fructose 1,6-bisphosphatase, fumarylacetoacetate hydrolase, glutamate 5-kinase, glutamate-ammonia-ligase, glutaredoxin, glycerol-3-phosphate dehydrogenase, glycine cleavage system H protein, GM02-LC38418-protein, heat shock protein, heat shock transcription factor, HesB/YadR/YfhF family protein, homocitrate synthase, hydrolase, isocitrate dehydrogenase, lipoprotein precursor, malate dehydrogenase, malonyl CoA-acyl carrier protein transacylase, metal-dependent hydrolase, molecular chaperone, molecular chaperone portein, monothiol glutaredoxin, multiple antibiotic resistance protein, murein transglycosylase, N-acetyl-gamma-glutamyl-phosphate reductase, NADP-dependent malic enzyme, outer-membrane lipoprotein precursor, oxidoreductase, permease protein of ABC transporter, phage-related repressor protein, phosphatase, phosphate import ATP-binding protein, phosphatidylserine synthase, phospho-2-dehydro-3-deoxyheptonate aldolase, phosphoadenosine phosphosulfate reductase, phosphotransferase, phosphotransferase system component, polygalacturonase, protein kinase, protein phosphatase, purine nucleoside phosphorylase, pyridoxamine kinase, recombinase A, sec-independent protein translocase, Sec-independent protein translocase subunit, sensor histidine kinase, sensor kinase, sll0281-protein, sll0406-protein, small nuclear ribonucleoprotein, sodium/proton antiporter, squalene monooxygenase, sugar transport system permease protein, threonine dehydratase, transcription factor, transcriptional regulator, transcriptional regulator protein, transport protein, tryptophan synthase alpha chain, TTC1386-protein, vacuolar processing enzyme, ycl027w-protein, ycl049c-protein, ydr273w-protein, ydr339c-protein, YFL019C-protein, ygl237c-protein, YGR104C-protein, yhr033w-protein, YJL127W-A-protein, ylr326w-protein, yml083c-protein, yml084w-protein, and ynl321w-protein, or of a polypeptide as indicated in the respective line in Table II, application no. 12, columns 5 or 8, or its homologs or fragments, and conferring the production of or an increase in 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid, respectively; and/or
(b) stabilizing a mRNA conferring the generation or increased expression of a FCRP, e.g. protein encoded by the nucleic acid molecule of the invention or its homologs or fragments, or of a mRNA encoding the polypeptide of the present invention having the herein-mentioned activity selected from the group consisting of said activities mentioned in (a) and conferring the production of or an increase in 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid, respectively; and/or
(c) increasing the specific activity of a protein conferring the increased expression of a FCRP, e.g. a protein encoded by the nucleic acid molecule of the invention or of the polypeptide of the present invention having herein-mentioned 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid generating or increasing activity, respectively, e.g. of a polypeptide having the activity of a protein as indicated in the respective line in Table II, application no. 12, columns 5 or 8, or its homologs or fragments, or decreasing the inhibitory regulation of the polypeptide of the invention; and/or
(d) generating or increasing the expression of an endogenous or artificial transcription factor mediating the expression of a protein conferring the generation or increased expression of a FCRP, e.g. a protein encoded by the nucleic acid molecule of the invention or of the polypeptide of the invention having herein-mentioned activity selected from the group consisting of said activities mentioned in (a) and conferring a 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid increasing activity, respectively, e.g. of a polypeptide having the activity of a protein as indicated in the respective line in Table II, application no. 12, columns 5 or 8 or its homologs; and/or
(e) stimulating activity of a protein conferring the increased expression of a FCRP, e.g. a protein encoded by the nucleic acid molecule of the present invention or a polypeptide of the present invention having herein-mentioned activity selected from the group consisting of said activities mentioned in (a) and conferring a 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid increasing activity, respectively, e.g. of a polypeptide having the activity of a protein as indicated in the respective line in Table II, application no. 12, columns 5 or 8, or its homologs or fragments, by adding one or more exogenous inducing factors to the non-human organism or parts thereof; and/or
(f) expressing a transgenic gene encoding a protein conferring the increased expression of a FCRP, e.g. a polypeptide encoded by the nucleic acid molecule of the present invention or a polypeptide of the present invention, having herein-mentioned activity selected from the group consisting of said activities mentioned in (a) and conferring a 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid increasing activity, respectively, e.g. of a polypeptide having the activity of a protein as indicated in the respective line in Table II, application no. 12, columns 5 or 8, or its homologs or fragments, and/or
(g) increasing the copy number of a gene conferring the increased expression of a nucleic acid molecule encoding a FCRP, e.g. a polypeptide encoded by the nucleic acid molecule of the invention or the polypeptide of the invention having herein-mentioned activity selected from the group consisting of said activities mentioned in (a) and conferring a 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid increasing activity, respectively, e.g. of a polypeptide having the activity of a protein as indicated in the respective line in Table II, application no. 12, columns 5 or 8, or its homologs or fragments; and/or
(h) increasing the expression of the endogenous gene encoding the FCRP, polypeptide of the invention, e.g. a polypeptide encoded by the nucleic acid molecule of the invention or the polypeptide of the invention having herein-mentioned activity selected from the group consisting of said activities mentioned in (a) and conferring a 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid; increasing activity, respectively, e.g. of a polypeptide having the activity of a protein as indicated in the respective line in Table II, application no. 12, columns 5 or 8, or its homologs or fragments, by adding positive expression or removing negative expression elements, e.g. homologous recombination can be used to either introduce positive regulatory elements like for plants the 35S enhancer into the promoter or to remove repressor elements form regulatory regions. Further gene conversion methods can be used to disrupt repressor elements or to enhance to activity of positive elements. Positive elements can be randomly introduced in plants by T-DNA or transposon mutagenesis and lines can be identified in which the positive elements have be integrated near to a gene of the invention, the expression of which is thereby enhanced; and/or
(i) modulating growth conditions of the non-human organism in such a manner, that the expression or activity of the gene encoding the FCRP, e.g. a protein of the invention or the protein itself is enhanced, for example microorganisms or plants can be grown for example under a higher temperature regime leading to an enhanced expression of heat shock proteins, which can lead to an enhanced 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid production, respectively; and/or
(j) selecting of non-human organisms with especially high activity of the proteins of the invention from natural or from mutagenized resources and breeding them into the target organisms, e.g. the elite crops; and/or
(k) directing a protein encoded by the nucleic acid molecule of the invention or of the poly-peptide of the present invention having herein-mentioned activity selected from the group consisting of said activities mentioned in (a) and conferring a 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid increasing activity, respectively, e.g. of a polypeptide having the activity of a protein as indicated in the respective line in Table II, application no. 12, columns 5 or 8, or its homologs or fragments, if for the corresponding nucleic acid molecule in the respective line in column 6 of Table I the term “plastidic” is indicated, to the plastids by the addition of a plastidial targeting sequence or if for the corresponding nucleic acid molecule in the respective line in column 6 of Table I the term “mitochondrial” is indicated, to the mitochondria by the addition of a mitochondrial targeting sequence; and/or
(l) generating the expression of a protein encoded by the nucleic acid molecule of the invention or of the polypeptide of the present invention having herein-mentioned activity selected from the group consisting of said activities mentioned in (a) and conferring a 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid increasing activity, respectively, e.g. of a polypeptide having the activity of a protein as indicated in the respective line in Table II, application no. 12, columns 5 or 8, or its homologs or fragments, if for the corresponding nucleic acid molecule in the respective line in column 6 of Table I the term “plastidic” or “mitochondrial” is indicated, in these organelles by the stable or transient transformation, advantageously stable transformation, of organelles, preferably plastids or mitochondria, with an inventive nucleic acid sequence preferably in form of an expression cassette containing said sequence leading to the expression of the nucleic acids or polypeptides of the invention in the respective organelle; and/or
(m) generating the expression of a protein encoded by the nucleic acid molecule of the invention or of the polypeptide of the present invention having herein-mentioned activity selected from the group consisting of said activities mentioned in (a) and conferring a 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, and/or oleic acid increasing activity, respectively, e.g. of a polypeptide having the activity of a protein as indicated in the respective line in Table II, application no. 12, columns 5 or 8, or its homologs or fragments, if for the corresponding nucleic acid molecule in the respective line in column 6 of Table I the term “plastidic” or “mitochondrial” is indicated, in these organelles by integration of a nucleic acid of the invention into the genome of the respective organelle under control of preferable a promoter selective for the respective organelle.

Preferably, said mRNA is the coding region of the nucleic acid molecule of the present invention and/or the protein conferring the increased expression of a protein encoded by the coding region of the nucleic acid molecule of the present invention alone or linked to a transit nucleic acid sequence or transit peptide encoding nucleic acid sequence or the polypeptide having the herein mentioned activity, e.g. conferring the generation of or increase of 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid, respectively, after increasing the expression or activity of the encoded polypeptide, non-targeted or in organelles such as plastids and/or mitochondria, preferably plastids, or having the activity of a polypeptide having an activity as the protein as shown in the respective line in Table II, application no. 12, column 3, or its homologs. Preferably the increase of 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid, respectively, takes place non-targeted or in plastids and/or mitochondria, preferably non-targeted or in plastids.

[0113.1.8.12] to [0122.1.8.12] for the disclosure of these paragraphs see [0113.1.8.8] to [0122.1.8.8] above.

The activation of an endogenous polypeptide having above-mentioned activity, e.g. having the activity of a protein as shown in the respective line in Table II, application no. 12, column 3 or of the polypeptide of the invention, e.g. conferring the increase of the fine chemical 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid, respectively, by increase of expression or activity in the cytoplasm, and/or in the cytosol, and/or in an organelle, such as plastids or mitochondria, can also be increased by introducing a synthetic transcription factor, which binds close to the coding region of the gene encoding the protein as shown in the respective line in Table II, application no. 12, column 5 or 8, or homologs or fragments thereof, and activates its transcription. A chimeric zinc finger protein can be constructed, which comprises a specific DNA-binding domain and an activation domain as e.g. the VP16 domain of Herpes Simplex virus. The specific binding domain can bind to the regulatory region of the gene encoding the protein as shown in the respective line in Table II, application no. 12, column 5 or 8, or homologs or fragments thereof. The expression of the chimeric transcription factor in a non-human organism, in particular in a plant, leads to a specific expression of the protein as shown in the respective line in Table II, application no. 12, column 5 or 8, or homologs or fragments thereof, see e.g. in WO01/52620, Oriz, Proc. Natl. Acad. Sci. USA 99, 13290 (2002), or Guan, Proc. Natl. Acad. Sci. USA 99, 13296 (2002).

[0124.1.8.12] to [0127.1.8.12] for the disclosure of these paragraphs see [0124.1.8.8] to [0127.1.8.8] above.

Owing to the introduction of a gene or a plurality of genes conferring the expression of the nucleic acid molecule of the invention or the polypeptide of the invention, for example the nucleic acid construct mentioned below, or encoding the protein as shown in the respective line in Table II, application no. 12, column 5 or 8, or homologs or fragments thereof, into a non-human organism alone or in combination with other genes, it is possible not only to increase the biosynthetic flux towards the end product, but also to increase, modify or create de novo an advantageous, preferably novel metabolites composition in the non-human organism, e.g. an advantageous composition comprising a higher content of (from a viewpoint of nutritional physiology limited) 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid and if desired other fatty acids, and/or other metabo-lites, in free or bound form.

for the disclosure of this paragraph see [0129.1.8.8] above.

Accordingly, in one embodiment, the process according to the invention relates to a process, which comprises:
(a) providing a non-human organism, preferably a microorganism, a plant cell, a plant tissue, a plant or a part thereof, more preferably a microorganism, a plant tissue, a plant or a part thereof;
(b) increasing the activity of a protein as shown in the respective line in Table II, application no. 12, column 3 or of a polypeptide being encoded by the respective nucleic acid molecule of the present invention and described below, e.g. conferring an increase of the fine chemical 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid, respectively, in the non-human organism, preferably in the microorganism, the plant cell, the plant tissue, the plant or a part thereof, more preferably a microorganism, a plant tissue, a plant or a part thereof, especially cytoplasmic or in an organelle, like plastids or mitochondria,
(c) growing the non-human organism, preferably the microorganism, the plant cell, the plant tissue, the plant or a part thereof, under conditions which permit the production of said fine chemical in the non-human organism, preferably the microorganism, the plant cell, the plant tissue, the plant or a part thereof; and
(d) if desired, recovering, optionally isolating, said fine chemical, in free and/or bound form, and, optionally further free and/or bound fatty acids, and/or other metabolites synthesized by the non-human organism, the microorganism, the plant cell, the plant tissue, the plant or a part thereof.

[0131.1.8.12] to [0139.1.8.12] for the disclosure of these paragraphs see [0131.1.8.8] to [0139.1.8.8] above.

In another embodiment, the invention relates to a (isolated) nucleic acid molecule, which comprises a nucleic acid molecule selected from the group consisting of:
(a) a nucleic acid molecule encoding a polypeptide shown in Table II, application no. 12, column 5 or 8, preferably shown in Table II B, application no. 12, column 8, or a homolog or a fragment thereof;
(b) a nucleic acid molecule shown in Table I, application no. 12, column 5 or 8, preferably shown in Table I B, application no. 12, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 12, preferably in column 8 of Table II B, application no. 12;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 12, preferably shown in column 8 of Table I B, application no. 12, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 12;
(i) a nucleic acid molecule encoding a polypeptide having the activity represented by a respective protein as depicted in column 5 of Table II, application no. 12;
(j) a nucleic acid molecule comprising a polynucleotide which is obtained by amplifying a cDNA library or a genomic library using the primers in Table III, application no. 12, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 12.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II application no. 12 and confers a generation or an increase of the respective fine chemical in a non-human organism or a part thereof as compared to a corresponding wild-type non-human organism or part thereof.

In another embodiment, the invention relates to a (isolated) nucleic acid molecule, which comprises a nucleic acid molecule selected from the group consisting of:
(a) a nucleic acid molecule encoding a polypeptide shown in Table II, column 5 or 8, application no. 12, preferably shown in Table II A, application no. 12, in column 5 or in Table II A, application no. 12, column 8 or in Table II B, application no. 12, column 8, or a homolog or a fragment thereof;
(b) a nucleic acid molecule shown in Table I, application no. 12, column 5 or 8, preferably shown in Table I A, application no. 12, in column 5 or in Table I A, application no. 12, column 8 or in Table I B, application no. 12, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 12, preferably shown in Table II A, application no. 12, in column 5 or in Table II A, application no. 12, column 8 or in Table II B, application no. 12, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 12, preferably shown in Table I A, application no. 12, in column 5 or in Table I A, application no. 12, column 8 or in Table I B column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 12;
(i) a nucleic acid molecule encoding a polypeptide having the activity represented by a respective protein as depicted in column 5 of Table II, application no. 12;
(j) a nucleic acid molecule comprising a polynucleotide which is obtained by amplifying a cDNA library or a genomic library using the primers in Table III, application no. 12, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt,500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto;
   whereby, preferably, the nucleic acid molecule according to (a), (b), (c), (d), (e), (f), (g), (h), (i), (j) or (k) distinguishes over the sequence depicted in Table I A and/or I B, application no. 12, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 12, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 12, column 5 or 8, or the coding region thereof, respectively. In a further embodiment the nucleic acid molecule does not encode the polypeptide sequence shown in Table II A and/or II B, application no. 12, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 12, column 5 or 8 and does not encode a protein of the sequence shown in Table II A and/or II B, application no. 12, column 5 or 8.

Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 12, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 12, column 5 or 8 but less than 100%, preferably less than 99.999%, 99.99%, 99.9%, 99%, 98%, 97%, 96% or 95% identical to the sequence shown in Table II A and/or II B, application no. 12, column 5 or 8.

(All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 12.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II, application no. 12, and confers a generation or an increase of the respective fine chemical in a non-human organism or a part thereof as compared to a corresponding wild-type non-human organism or part thereof.

[0144.1.8.12] to [0155.1.8.12] for the disclosure of these paragraphs see [0144.1.8.8] to [0155.1.8.8] above.

In a preferred embodiment a nucleic acid construct, for example an expression cassette, comprises upstream, i.e. at the 5′ end of the encoding sequence, a promoter and downstream, i.e. at the 3′ end, a polyadenylation signal and optionally other regulatory elements which are operably linked to the intervening encoding sequence with one of the nucleic acids as depicted in the respective line in Table I, application no. 12, column 5 or 8, preferably the coding region thereof, or a homolog or a fragment thereof. By an operable linkage is meant the sequential arrangement of promoter, encoding sequence, terminator, optionally other regulatory elements and optionally targeting sequences in such a way that each of the regulatory elements/targeting sequence can fulfill its function in the expression of the encoding sequence in due manner. In one embodiment the sequences preferred for operable linkage are targeting sequences for ensuring subcellular localization in organelles, like plastids or mitochondria. However, targeting sequences for ensuring subcellular localization in the endoplasmic reticulum (=ER), in the nucleus, in oil corpuscles or other compartments may also be employed as well as translation promoters such as the 5′ lead sequence in tobacco mosaic virus (Gallie et al., Nucl. Acids Res. 15, 8693 (1987)).

for the disclosure of this paragraph see [0157.1.8.8] above

In an embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 12, column 5 or 8, preferably shown in Table II A, application no. 12, column 5, or in Table II A, application no. 12, column 8, or in Table II B, application no. 12, column 8, or a homolog or a fragment thereof;
   (b) a nucleic acid molecule shown in Table I, application no. 12, column 5 or 8, preferably shown in Table I A, application no. 12, column 5, or in Table I A, application no. 12, column 8, or in Table I B, application no. 12, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 12, preferably shown in Table II A, application no. 12, column 5, or in Table II A, application no. 12, column 8, or in Table II B, application no. 12, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 12, preferably shown in Table I A, application no. 12, column 5, or in Table I A, application no. 12, column 8, or in Table I B, application no. 12, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 12;
   (i) a nucleic acid molecule encoding a polypeptide having the activity represented by a respective protein as depicted in column 5 of Table II, application no. 12;
   (j) a nucleic acid molecule comprising a polynucleotide which is obtained by amplifying a cDNA library or a genomic library using the primers in Table III, application no. 12, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto.
      and, optionally
3) nucleic acid molecule encoding a transit peptide, preferably encoding a plastidal transit peptide or a mitochondrial transit peptide;
   which are operable linked.
   (All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 12.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 12 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 12, in the same line as of the respective nucleic acid molecule according to 2) “plastidic” is depicted, or encoding a mitochondrial transit peptide, especially in case in column 6 of Table I, application no. 12, in the same line as of the respective nucleic acid molecule according to 2) “mitochondrial” is depicted.

In another embodiment thereof the expression cassette does not comprise 3) a nucleic acid molecule encoding a transit peptide, especially in case in column 6 of Table I, application no. 12, in the same line as of the respective nucleic acid molecule according to 2) “non-targeted” is depicted.

In a further embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 12, column 5 or 8, preferably shown in Table II A, application no. 12, column 5, or in Table II A, application no. 12, column 8, or in Table II B, application no. 12, column 8, or a homolog or a fragment thereof;
   (b) a nucleic acid molecule shown in Table I, application no. 12, column 5 or 8, preferably shown in Table I A, application no. 12, column 5, or in Table I A, application no. 12, column 8, or in Table I B, application no. 12, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 12, preferably shown in Table II A, application no. 12, column 5, or in Table II A, application no. 12, column 8, or in Table II B, application no. 12, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 12, preferably shown in Table I A, application no. 12, column 5, or in Table I A, application no. 12, column 8,or in Table I B, application no. 12, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 12;
   (i) a nucleic acid molecule encoding a polypeptide having the activity represented by a respective protein as depicted in column 5 of Table II, application no. 12;
   (j) a nucleic acid molecule comprising a polynucleotide which is obtained by amplifying a cDNA library or a genomic library using the primers in Table III, application no. 12, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, optionally
3) nucleic acid molecule encoding a transit peptide, preferably encoding a plastidal transit peptide or a mitochondrial transit peptide;
   which are operable linked and
   whereby, preferably, the nucleic acid molecule according to 2 (a), (b), (c), (d), (e), (f), (g), (h), (i), (j) or (k) distinguishes over the sequence depicted in Table I A and/or I B, application no. 12, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention according to 2) does not consist of the sequence shown in Table I A and/or I B, application no. 12, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 12, column 5 or 8, or the coding region thereof, respectively. In a further embodiment the nucleic acid molecule does not encode the polypeptide sequence shown in Table II A and/or II B, application no. 12, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 12, column 5 or 8 and does not encode a protein of the sequence shown in Table II A and/or II B, application no. 12, column 5 or 8.

Accordingly, in one embodiment, the protein encoded by a sequence of a nucleic acid according to 2(a), (b), (c), (d), (e), (f), (g), (h), (i), (j) or (k) does not consist of the sequence shown in Table I A and/or I B, application no. 12, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 12, column 5 or 8 but less than 100%, 99.999%, 99.99%, 99.9%, 99%, 98%, 97%, 96% or 95% identical to the sequence shown in Table II A and/or II B, application no. 12, column 5 or 8.

(All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 12.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 12 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 12, in the same line as of the respective nucleic acid molecule according to 2) “plastidic” is depicted, or encoding a mitochondrial transit peptide, especially in case in column 6 of Table I, application no. 12, in the same line as of the respective nucleic acid molecule according to 2) “mitochondrial” is depicted.

In another embodiment thereof the expression cassette does not comprise 3) a nucleic acid molecule encoding a transit peptide, especially in case in column 6 of Table I, application no. 12, in the same line as of the respective nucleic acid molecule according to 2) “non-targeted” is depicted.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 113651, or a homolog or fragment thereof, or a homolog thereof being depicted in Table I, application no. 12, column 8, in the same line as SEQ ID NO. 113651, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 113651 is depicted, plastidic is mentioned, or a mitochondrial transit peptide in case in Table I, application no. 12, in column 6 in the same line as SEQ ID NO. 113651 is depicted, mitochondrial is mentioned; which is operable linked, too.

In another embodiment thereof the expression cassette does not comprise an additional transit peptide in case in Table I, application no. 12, in column 6 in the same line as SEQ ID NO. 113651 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 1061, or a homolog or fragment thereof, or a homolog thereof being depicted in Table I, application no. 12, column 8, in the same line as SEQ ID NO. 1061, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 1061 is depicted, plastidic is mentioned, or a mitochondrial transit peptide in case in Table I, application no. 12, in column 6 in the same line as SEQ ID NO. 1061 is depicted, mitochondrial is mentioned; which is operable linked, too.

In another embodiment thereof the expression cassette does not comprise an additional transit peptide in case in Table I, application no. 12, in column 6 in the same line as SEQ ID NO. 1061 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 19419, or a homolog or fragment thereof, or a homolog thereof being depicted in Table I, application no. 12, column 8, in the same line as SEQ ID NO. 19419, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 19419 is depicted, plastidic is mentioned, or a mitochondrial transit peptide in case in Table I, application no. 12, in column 6 in the same line as SEQ ID NO. 19419 is depicted, mitochondrial is mentioned; which is operable linked, too.

In another embodiment thereof the expression cassette does not comprise an additional transit peptide in case in Table I, application no. 12, in column 6 in the same line as SEQ ID NO. 19419 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 98522, or a homolog or fragment thereof, or a homolog thereof being depicted in Table I, application no. 12, column 8, in the same line as SEQ ID NO. 98522, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 98522 is depicted, plastidic is mentioned, or a mitochondrial transit peptide in case in Table I, application no. 12, in column 6 in the same line as SEQ ID NO. 98522 is depicted, mitochondrial is mentioned; which is operable linked, too.

In another embodiment thereof the expression cassette does not comprise an additional transit peptide in case in Table I, application no. 12, in column 6 in the same line as SEQ ID NO. 98522 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68657, or a homolog or fragment thereof, or a homolog thereof being depicted in Table I, application no. 12, column 8, in the same line as SEQ ID NO. 68657, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 68657 is depicted, plastidic is mentioned, or a mitochondrial transit peptide in case in Table I, application no. 12, in column 6 in the same line as SEQ ID NO. 68657 is depicted, mitochondrial is mentioned; which is operable linked, too.

In another embodiment thereof the expression cassette does not comprise an additional transit peptide in case in Table I, application no. 12, in column 6 in the same line as SEQ ID NO. 68657 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 1696, or a homolog or fragment thereof, or a homolog thereof being depicted in Table I, application no. 12, column 8, in the same line as SEQ ID NO. 1696, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 1696 is depicted, plastidic is mentioned, or a mitochondrial transit peptide in case in Table I, application no. 12, in column 6 in the same line as SEQ ID NO. 1696 is depicted, mitochondrial is mentioned; which is operable linked, too.

In another embodiment thereof the expression cassette does not comprise an additional transit peptide in case in Table I, application no. 12, in column 6 in the same line as SEQ ID NO. 1696 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 22832, or a homolog or fragment thereof, or a homolog thereof being depicted in Table I, application no. 12, column 8, in the same line as SEQ ID NO. 22832, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 22832 is depicted, plastidic is mentioned, or a mitochondrial transit peptide in case in Table I, application no. 12, in column 6 in the same line as SEQ ID NO. 22832 is depicted, mitochondrial is mentioned; which is operable linked, too.

In another embodiment thereof the expression cassette does not comprise an additional transit peptide in case in Table I, application no. 12, in column 6 in the same line as SEQ ID NO. 22832 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 2935, or a homolog or fragment thereof, or a homolog thereof being depicted in Table I, application no. 12, column 8, in the same line as SEQ ID NO. 2935, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 2935 is depicted, plastidic is mentioned, or a mitochondrial transit peptide in case in Table I, application no. 12, in column 6 in the same line as SEQ ID NO. 2935 is depicted, mitochondrial is mentioned; which is operable linked, too.

In another embodiment thereof the expression cassette does not comprise an additional transit peptide in case in Table I, application no. 12, in column 6 in the same line as SEQ ID NO. 2935 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 109717, or a homolog or fragment thereof, or a homolog thereof being depicted in Table I, application no. 12, column 8, in the same line as SEQ ID NO. 109717, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 109717 is depicted, plastidic is mentioned, or a mitochondrial transit peptide in case in Table I, application no. 12, in column 6 in the same line as SEQ ID NO. 109717 is depicted, mitochondrial is mentioned; which is operable linked, too.

In another embodiment thereof the expression cassette does not comprise an additional transit peptide in case in Table I, application no. 12, in column 6 in the same line as SEQ ID NO. 109717 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 23482, or a homolog or fragment thereof, or a homolog thereof being depicted in Table I, application no. 12, column 8, in the same line as SEQ ID NO. 23482, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 23482 is depicted, plastidic is mentioned, or a mitochondrial transit peptide in case in Table I, application no. 12, in column 6 in the same line as SEQ ID NO. 23482 is depicted, mitochondrial is mentioned; which is operable linked, too.

In another embodiment thereof the expression cassette does not comprise an additional transit peptide in case in Table I, application no. 12, in column 6 in the same line as SEQ ID NO. 23482 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 3279, or a homolog or fragment thereof, or a homolog thereof being depicted in Table I, application no. 12, column 8, in the same line as SEQ ID NO. 3279, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 3279 is depicted, plastidic is mentioned, or a mitochondrial transit peptide in case in Table I, application no. 12, in column 6 in the same line as SEQ ID NO. 3279 is depicted, mitochondrial is mentioned; which is operable linked, too.

In another embodiment thereof the expression cassette does not comprise an additional transit peptide in case in Table I, application no. 12, in column 6 in the same line as SEQ ID NO. 3279 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 23844, or a homolog or fragment thereof, or a homolog thereof being depicted in Table I, application no. 12, column 8, in the same line as SEQ ID NO. 23844, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 23844 is depicted, plastidic is mentioned, or a mitochondrial transit peptide in case in Table I, application no. 12, in column 6 in the same line as SEQ ID NO. 23844 is depicted, mitochondrial is mentioned; which is operable linked, too.

In another embodiment thereof the expression cassette does not comprise an additional transit peptide in case in Table I, application no. 12, in column 6 in the same line as SEQ ID NO. 23844 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68849, or a homolog or fragment thereof, or a homolog thereof being depicted in Table I, application no. 12, column 8, in the same line as SEQ ID NO. 68849, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 68849 is depicted, plastidic is mentioned, or a mitochondrial transit peptide in case in Table I, application no. 12, in column 6 in the same line as SEQ ID NO. 68849 is depicted, mitochondrial is mentioned; which is operable linked, too.

In another embodiment thereof the expression cassette does not comprise an additional transit peptide in case in Table I, application no. 12, in column 6 in the same line as SEQ ID NO. 68849 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4102, or a homolog or fragment thereof, or a homolog thereof being depicted in Table I, application no. 12, column 8, in the same line as SEQ ID NO. 4102, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 4102 is depicted, plastidic is mentioned, or a mitochondrial transit peptide in case in Table I, application no. 12, in column 6 in the same line as SEQ ID NO. 4102 is depicted, mitochondrial is mentioned; which is operable linked, too.

In another embodiment thereof the expression cassette does not comprise an additional transit peptide in case in Table I, application no. 12, in column 6 in the same line as SEQ ID NO. 4102 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4348, or a homolog or fragment thereof, or a homolog thereof being depicted in Table I, application no. 12, column 8, in the same line as SEQ ID NO. 4348, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 4348 is depicted, plastidic is mentioned, or a mitochondrial transit peptide in case in Table I, application no. 12, in column 6 in the same line as SEQ ID NO. 4348 is depicted, mitochondrial is mentioned; which is operable linked, too.

In another embodiment thereof the expression cassette does not comprise an additional transit peptide in case in Table I, application no. 12, in column 6 in the same line as SEQ ID NO. 4348 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 122565, or a homolog or fragment thereof, or a homolog thereof being depicted in Table I, application no. 12, column 8, in the same line as SEQ ID NO. 122565, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 122565 is depicted, plastidic is mentioned, or a mitochondrial transit peptide in case in Table I, application no. 12, in column 6 in the same line as SEQ ID NO. 122565 is depicted, mitochondrial is mentioned; which is operable linked, too.

In another embodiment thereof the expression cassette does not comprise an additional transit peptide in case in Table I, application no. 12, in column 6 in the same line as SEQ ID NO. 122565 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4904, or a homolog or fragment thereof, or a homolog thereof being depicted in Table I, application no. 12, column 8, in the same line as SEQ ID NO. 4904, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 4904 is depicted, plastidic is mentioned, or a mitochondrial transit peptide in case in Table I, application no. 12, in column 6 in the same line as SEQ ID NO. 4904 is depicted, mitochondrial is mentioned; which is operable linked, too.

In another embodiment thereof the expression cassette does not comprise an additional transit peptide in case in Table I, application no. 12, in column 6 in the same line as SEQ ID NO. 4904 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 5318, or a homolog or fragment thereof, or a homolog thereof being depicted in Table I, application no. 12, column 8, in the same line as SEQ ID NO. 5318, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 5318 is depicted, plastidic is mentioned, or a mitochondrial transit peptide in case in Table I, application no. 12, in column 6 in the same line as SEQ ID NO. 5318 is depicted, mitochondrial is mentioned; which is operable linked, too.

In another embodiment thereof the expression cassette does not comprise an additional transit peptide in case in Table I, application no. 12, in column 6 in the same line as SEQ ID NO. 5318 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 122803, or a homolog or fragment thereof, or a homolog thereof being depicted in Table I, application no. 12, column 8, in the same line as SEQ ID NO. 122803, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 122803 is depicted, plastidic is mentioned, or a mitochondrial transit peptide in case in Table I, application no. 12, in column 6 in the same line as SEQ ID NO. 122803 is depicted, mitochondrial is mentioned; which is operable linked, too.

In another embodiment thereof the expression cassette does not comprise an additional transit peptide in case in Table I, application no. 12, in column 6 in the same line as SEQ ID NO. 122803 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 70583, or a homolog or fragment thereof, or a homolog thereof being depicted in Table I, application no. 12, column 8, in the same line as SEQ ID NO. 70583, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 70583 is depicted, plastidic is mentioned, or a mitochondrial transit peptide in case in Table I, application no. 12, in column 6 in the same line as SEQ ID NO. 70583 is depicted, mitochondrial is mentioned; which is operable linked, too.

In another embodiment thereof the expression cassette does not comprise an additional transit peptide in case in Table I, application no. 12, in column 6 in the same line as SEQ ID NO. 70583 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 26196, or a homolog or fragment thereof, or a homolog thereof being depicted in Table I, application no. 12, column 8, in the same line as SEQ ID NO. 26196, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 26196 is depicted, plastidic is mentioned, or a mitochondrial transit peptide in case in Table I, application no. 12, in column 6 in the same line as SEQ ID NO. 26196 is depicted, mitochondrial is mentioned; which is operable linked, too.

In another embodiment thereof the expression cassette does not comprise an additional transit peptide in case in Table I, application no. 12, in column 6 in the same line as SEQ ID NO. 26196 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 114198, or a homolog or fragment thereof, or a homolog thereof being depicted in Table I, application no. 12, column 8, in the same line as SEQ ID NO. 114198, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 114198 is depicted, plastidic is mentioned, or a mitochondrial transit peptide in case in Table I, application no. 12, in column 6 in the same line as SEQ ID NO. 114198 is depicted, mitochondrial is mentioned; which is operable linked, too.

In another embodiment thereof the expression cassette does not comprise an additional transit peptide in case in Table I, application no. 12, in column 6 in the same line as SEQ ID NO. 114198 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6040, or a homolog or fragment thereof, or a homolog thereof being depicted in Table I, application no. 12, column 8, in the same line as SEQ ID NO. 6040, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 6040 is depicted, plastidic is mentioned, or a mitochondrial transit peptide in case in Table I, application no. 12, in column 6 in the same line as SEQ ID NO. 6040 is depicted, mitochondrial is mentioned; which is operable linked, too.

In another embodiment thereof the expression cassette does not comprise an additional transit peptide in case in Table I, application no. 12, in column 6 in the same line as SEQ ID NO. 6040 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 71325, or a homolog or fragment thereof, or a homolog thereof being depicted in Table I, application no. 12, column 8, in the same line as SEQ ID NO. 71325, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 71325 is depicted, plastidic is mentioned, or a mitochondrial transit peptide in case in Table I, application no. 12, in column 6 in the same line as SEQ ID NO. 71325 is depicted, mitochondrial is mentioned; which is operable linked, too.

In another embodiment thereof the expression cassette does not comprise an additional transit peptide in case in Table I, application no. 12, in column 6 in the same line as SEQ ID NO. 71325 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 28040, or a homolog or fragment thereof, or a homolog thereof being depicted in Table I, application no. 12, column 8, in the same line as SEQ ID NO. 28040, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 28040 is depicted, plastidic is mentioned, or a mitochondrial transit peptide in case in Table I, application no. 12, in column 6 in the same line as SEQ ID NO. 28040 is depicted, mitochondrial is mentioned; which is operable linked, too.

In another embodiment thereof the expression cassette does not comprise an additional transit peptide in case in Table I, application no. 12, in column 6 in the same line as SEQ ID NO. 28040 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 122948, or a homolog or fragment thereof, or a homolog thereof being depicted in Table I, application no. 12, column 8, in the same line as SEQ ID NO. 122948, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 122948 is depicted, plastidic is mentioned, or a mitochondrial transit peptide in case in Table I, application no. 12, in column 6 in the same line as SEQ ID NO. 122948 is depicted, mitochondrial is mentioned; which is operable linked, too.

In another embodiment thereof the expression cassette does not comprise an additional transit peptide in case in Table I, application no. 12, in column 6 in the same line as SEQ ID NO. 122948 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 29500, or a homolog or fragment thereof, or a homolog thereof being depicted in Table I, application no. 12, column 8, in the same line as SEQ ID NO. 29500, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 29500 is depicted, plastidic is mentioned, or a mitochondrial transit peptide in case in Table I, application no. 12, in column 6 in the same line as SEQ ID NO. 29500 is depicted, mitochondrial is mentioned; which is operable linked, too.

In another embodiment thereof the expression cassette does not comprise an additional transit peptide in case in Table I, application no. 12, in column 6 in the same line as SEQ ID NO. 29500 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 114231, or a homolog or fragment thereof, or a homolog thereof being depicted in Table I, application no. 12, column 8, in the same line as SEQ ID NO. 114231, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 114231 is depicted, plastidic is mentioned, or a mitochondrial transit peptide in case in Table I, application no. 12, in column 6 in the same line as SEQ ID NO. 114231 is depicted, mitochondrial is mentioned; which is operable linked, too.

In another embodiment thereof the expression cassette does not comprise an additional transit peptide in case in Table I, application no. 12, in column 6 in the same line as SEQ ID NO. 114231 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 72520, or a homolog or fragment thereof, or a homolog thereof being depicted in Table I, application no. 12, column 8, in the same line as SEQ ID NO. 72520, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 72520 is depicted, plastidic is mentioned, or a mitochondrial transit peptide in case in Table I, application no. 12, in column 6 in the same line as SEQ ID NO. 72520 is depicted, mitochondrial is mentioned; which is operable linked, too.

In another embodiment thereof the expression cassette does not comprise an additional transit peptide in case in Table I, application no. 12, in column 6 in the same line as SEQ ID NO. 72520 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6510, or a homolog or fragment thereof, or a homolog thereof being depicted in Table I, application no. 12, column 8, in the same line as SEQ ID NO. 6510, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 6510 is depicted, plastidic is mentioned, or a mitochondrial transit peptide in case in Table I, application no. 12, in column 6 in the same line as SEQ ID NO. 6510 is depicted, mitochondrial is mentioned; which is operable linked, too.

In another embodiment thereof the expression cassette does not comprise an additional transit peptide in case in Table I, application no. 12, in column 6 in the same line as SEQ ID NO. 6510 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 32037, or a homolog or fragment thereof, or a homolog thereof being depicted in Table I, application no. 12, column 8, in the same line as SEQ ID NO. 32037, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 32037 is depicted, plastidic is mentioned, or a mitochondrial transit peptide in case in Table I, application no. 12, in column 6 in the same line as SEQ ID NO. 32037 is depicted, mitochondrial is mentioned; which is operable linked, too.

In another embodiment thereof the expression cassette does not comprise an additional transit peptide in case in Table I, application no. 12, in column 6 in the same line as SEQ ID NO. 32037 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 32308, or a homolog or fragment thereof, or a homolog thereof being depicted in Table I, application no. 12, column 8, in the same line as SEQ ID NO. 32308, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 32308 is depicted, plastidic is mentioned, or a mitochondrial transit peptide in case in Table I, application no. 12, in column 6 in the same line as SEQ ID NO. 32308 is depicted, mitochondrial is mentioned; which is operable linked, too.

In another embodiment thereof the expression cassette does not comprise an additional transit peptide in case in Table I, application no. 12, in column 6 in the same line as SEQ ID NO. 32308 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 123087, or a homolog or fragment thereof, or a homolog thereof being depicted in Table I, application no. 12, column 8, in the same line as SEQ ID NO. 123087, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 123087 is depicted, plastidic is mentioned, or a mitochondrial transit peptide in case in Table I, application no. 12, in column 6 in the same line as SEQ ID NO. 123087 is depicted, mitochondrial is mentioned; which is operable linked, too.

In another embodiment thereof the expression cassette does not comprise an additional transit peptide in case in Table I, application no. 12, in column 6 in the same line as SEQ ID NO. 123087 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 32648, or a homolog or fragment thereof, or a homolog thereof being depicted in Table I, application no. 12, column 8, in the same line as SEQ ID NO. 32648, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 32648 is depicted, plastidic is mentioned, or a mitochondrial transit peptide in case in Table I, application no. 12, in column 6 in the same line as SEQ ID NO. 32648 is depicted, mitochondrial is mentioned; which is operable linked, too.

In another embodiment thereof the expression cassette does not comprise an additional transit peptide in case in Table I, application no. 12, in column 6 in the same line as SEQ ID NO. 32648 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 34044, or a homolog or fragment thereof, or a homolog thereof being depicted in Table I, application no. 12, column 8, in the same line as SEQ ID NO. 34044, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 34044 is depicted, plastidic is mentioned, or a mitochondrial transit peptide in case in Table I, application no. 12, in column 6 in the same line as SEQ ID NO. 34044 is depicted, mitochondrial is mentioned; which is operable linked, too.

In another embodiment thereof the expression cassette does not comprise an additional transit peptide in case in Table I, application no. 12, in column 6 in the same line as SEQ ID NO. 34044 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 103958, or a homolog or fragment thereof, or a homolog thereof being depicted in Table I, application no. 12, column 8, in the same line as SEQ ID NO. 103958, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 103958 is depicted, plastidic is mentioned, or a mitochondrial transit peptide in case in Table I, application no. 12, in column 6 in the same line as SEQ ID NO. 103958 is depicted, mitochondrial is mentioned; which is operable linked, too.

In another embodiment thereof the expression cassette does not comprise an additional transit peptide in case in Table I, application no. 12, in column 6 in the same line as SEQ ID NO. 103958 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 123473, or a homolog or fragment thereof, or a homolog thereof being depicted in Table I, application no. 12, column 8, in the same line as SEQ ID NO. 123473, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 123473 is depicted, plastidic is mentioned, or a mitochondrial transit peptide in case in Table I, application no. 12, in column 6 in the same line as SEQ ID NO. 123473 is depicted, mitochondrial is mentioned; which is operable linked, too.

In another embodiment thereof the expression cassette does not comprise an additional transit peptide in case in Table I, application no. 12, in column 6 in the same line as SEQ ID NO. 123473 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 121111, or a homolog or fragment thereof, or a homolog thereof being depicted in Table I, application no. 12, column 8, in the same line as SEQ ID NO. 121111, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 121111 is depicted, plastidic is mentioned, or a mitochondrial transit peptide in case in Table I, application no. 12, in column 6 in the same line as SEQ ID NO. 121111 is depicted, mitochondrial is mentioned; which is operable linked, too.

In another embodiment thereof the expression cassette does not comprise an additional transit peptide in case in Table I, application no. 12, in column 6 in the same line as SEQ ID NO. 121111 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 7269, or a homolog or fragment thereof, or a homolog thereof being depicted in Table I, application no. 12, column 8, in the same line as SEQ ID NO. 7269, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 7269 is depicted, plastidic is mentioned, or a mitochondrial transit peptide in case in Table I, application no. 12, in column 6 in the same line as SEQ ID NO. 7269 is depicted, mitochondrial is mentioned; which is operable linked, too.

In another embodiment thereof the expression cassette does not comprise an additional transit peptide in case in Table I, application no. 12, in column 6 in the same line as SEQ ID NO. 7269 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 121637, or a homolog or fragment thereof, or a homolog thereof being depicted in Table I, application no. 12, column 8, in the same line as SEQ ID NO. 121637, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 121637 is depicted, plastidic is mentioned, or a mitochondrial transit peptide in case in Table I, application no. 12, in column 6 in the same line as SEQ ID NO. 121637 is depicted, mitochondrial is mentioned; which is operable linked, too.

In another embodiment thereof the expression cassette does not comprise an additional transit peptide in case in Table I, application no. 12, in column 6 in the same line as SEQ ID NO. 121637 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7333, or a homolog or fragment thereof, or a homolog thereof being depicted in Table I, application no. 12, column 8, in the same line as SEQ ID NO. 7333, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 7333 is depicted, plastidic is mentioned, or a mitochondrial transit peptide in case in Table I, application no. 12, in column 6 in the same line as SEQ ID NO. 7333 is depicted, mitochondrial is mentioned; which is operable linked, too.

In another embodiment thereof the expression cassette does not comprise an additional transit peptide in case in Table I, application no. 12, in column 6 in the same line as SEQ ID NO. 7333 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 35936, or a homolog or fragment thereof, or a homolog thereof being depicted in Table I, application no. 12, column 8, in the same line as SEQ ID NO. 35936, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 35936 is depicted, plastidic is mentioned, or a mitochondrial transit peptide in case in Table I, application no. 12, in column 6 in the same line as SEQ ID NO. 35936 is depicted, mitochondrial is mentioned; which is operable linked, too.

In another embodiment thereof the expression cassette does not comprise an additional transit peptide in case in Table I, application no. 12, in column 6 in the same line as SEQ ID NO. 35936 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 93678, or a homolog or fragment thereof, or a homolog thereof being depicted in Table I, application no. 12, column 8, in the same line as SEQ ID NO. 93678, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 93678 is depicted, plastidic is mentioned, or a mitochondrial transit peptide in case in Table I, application no. 12, in column 6 in the same line as SEQ ID NO. 93678 is depicted, mitochondrial is mentioned; which is operable linked, too.

In another embodiment thereof the expression cassette does not comprise an additional transit peptide in case in Table I, application no. 12, in column 6 in the same line as SEQ ID NO. 93678 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 123498, or a homolog or fragment thereof, or a homolog thereof being depicted in Table I, application no. 12, column 8, in the same line as SEQ ID NO. 123498, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 123498 is depicted, plastidic is mentioned, or a mitochondrial transit peptide in case in Table I, application no. 12, in column 6 in the same line as SEQ ID NO. 123498 is depicted, mitochondrial is mentioned; which is operable linked, too.

In another embodiment thereof the expression cassette does not comprise an additional transit peptide in case in Table I, application no. 12, in column 6 in the same line as SEQ ID NO. 123498 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 75807, or a homolog or fragment thereof, or a homolog thereof being depicted in Table I, application no. 12, column 8, in the same line as SEQ ID NO. 75807, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 75807 is depicted, plastidic is mentioned, or a mitochondrial transit peptide in case in Table I, application no. 12, in column 6 in the same line as SEQ ID NO. 75807 is depicted, mitochondrial is mentioned; which is operable linked, too.

In another embodiment thereof the expression cassette does not comprise an additional transit peptide in case in Table I, application no. 12, in column 6 in the same line as SEQ ID NO. 75807 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 124051, or a homolog or fragment thereof, or a homolog thereof being depicted in Table I, application no. 12, column 8, in the same line as SEQ ID NO. 124051, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 124051 is depicted, plastidic is mentioned, or a mitochondrial transit peptide in case in Table I, application no. 12, in column 6 in the same line as SEQ ID NO. 124051 is depicted, mitochondrial is mentioned; which is operable linked, too.

In another embodiment thereof the expression cassette does not comprise an additional transit peptide in case in Table I, application no. 12, in column 6 in the same line as SEQ ID NO. 124051 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 124127, or a homolog or fragment thereof, or a homolog thereof being depicted in Table I, application no. 12, column 8, in the same line as SEQ ID NO. 124127, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 124127 is depicted, plastidic is mentioned, or a mitochondrial transit peptide in case in Table I, application no. 12, in column 6 in the same line as SEQ ID NO. 124127 is depicted, mitochondrial is mentioned; which is operable linked, too.

In another embodiment thereof the expression cassette does not comprise an additional transit peptide in case in Table I, application no. 12, in column 6 in the same line as SEQ ID NO. 124127 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 118371, or a homolog or fragment thereof, or a homolog thereof being depicted in Table I, application no. 12, column 8, in the same line as SEQ ID NO. 118371, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 118371 is depicted, plastidic is mentioned, or a mitochondrial transit peptide in case in Table I, application no. 12, in column 6 in the same line as SEQ ID NO. 118371 is depicted, mitochondrial is mentioned; which is operable linked, too.

In another embodiment thereof the expression cassette does not comprise an additional transit peptide in case in Table I, application no. 12, in column 6 in the same line as SEQ ID NO. 118371 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 114552, or a homolog or fragment thereof, or a homolog thereof being depicted in Table I, application no. 12, column 8, in the same line as SEQ ID NO. 114552, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 114552 is depicted, plastidic is mentioned, or a mitochondrial transit peptide in case in Table I, application no. 12, in column 6 in the same line as SEQ ID NO. 114552 is depicted, mitochondrial is mentioned; which is operable linked, too.

In another embodiment thereof the expression cassette does not comprise an additional transit peptide in case in Table I, application no. 12, in column 6 in the same line as SEQ ID NO. 114552 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 37400, or a homolog or fragment thereof, or a homolog thereof being depicted in Table I, application no. 12, column 8, in the same line as SEQ ID NO. 37400, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 37400 is depicted, plastidic is mentioned, or a mitochondrial transit peptide in case in Table I, application no. 12, in column 6 in the same line as SEQ ID NO. 37400 is depicted, mitochondrial is mentioned; which is operable linked, too.

In another embodiment thereof the expression cassette does not comprise an additional transit peptide in case in Table I, application no. 12, in column 6 in the same line as SEQ ID NO. 37400 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 124509, or a homolog or fragment thereof, or a homolog thereof being depicted in Table I, application no. 12, column 8, in the same line as SEQ ID NO. 124509, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 124509 is depicted, plastidic is mentioned, or a mitochondrial transit peptide in case in Table I, application no. 12, in column 6 in the same line as SEQ ID NO. 124509 is depicted, mitochondrial is mentioned; which is operable linked, too.

In another embodiment thereof the expression cassette does not comprise an additional transit peptide in case in Table I, application no. 12, in column 6 in the same line as SEQ ID NO. 124509 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 98778, or a homolog or fragment thereof, or a homolog thereof being depicted in Table I, application no. 12, column 8, in the same line as SEQ ID NO. 98778, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 98778 is depicted, plastidic is mentioned, or a mitochondrial transit peptide in case in Table I, application no. 12, in column 6 in the same line as SEQ ID NO. 98778 is depicted, mitochondrial is mentioned; which is operable linked, too.

In another embodiment thereof the expression cassette does not comprise an additional transit peptide in case in Table I, application no. 12, in column 6 in the same line as SEQ ID NO. 98778 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 99188, or a homolog or fragment thereof, or a homolog thereof being depicted in Table I, application no. 12, column 8, in the same line as SEQ ID NO. 99188, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 99188 is depicted, plastidic is mentioned, or a mitochondrial transit peptide in case in Table I, application no. 12, in column 6 in the same line as SEQ ID NO. 99188 is depicted, mitochondrial is mentioned; which is operable linked, too.

In another embodiment thereof the expression cassette does not comprise an additional transit peptide in case in Table I, application no. 12, in column 6 in the same line as SEQ ID NO. 99188 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 124557, or a homolog or fragment thereof, or a homolog thereof being depicted in Table I, application no. 12, column 8, in the same line as SEQ ID NO. 124557, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 124557 is depicted, plastidic is mentioned, or a mitochondrial transit peptide in case in Table I, application no. 12, in column 6 in the same line as SEQ ID NO. 124557 is depicted, mitochondrial is mentioned; which is operable linked, too.

In another embodiment thereof the expression cassette does not comprise an additional transit peptide in case in Table I, application no. 12, in column 6 in the same line as SEQ ID NO. 124557 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38266, or a homolog or fragment thereof, or a homolog thereof being depicted in Table I, application no. 12, column 8, in the same line as SEQ ID NO. 38266, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 38266 is depicted, plastidic is mentioned, or a mitochondrial transit peptide in case in Table I, application no. 12, in column 6 in the same line as SEQ ID NO. 38266 is depicted, mitochondrial is mentioned; which is operable linked, too.

In another embodiment thereof the expression cassette does not comprise an additional transit peptide in case in Table I, application no. 12, in column 6 in the same line as SEQ ID NO. 38266 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115056, or a homolog or fragment thereof, or a homolog thereof being depicted in Table I, application no. 12, column 8, in the same line as SEQ ID NO. 115056, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 115056 is depicted, plastidic is mentioned, or a mitochondrial transit peptide in case in Table I, application no. 12, in column 6 in the same line as SEQ ID NO. 115056 is depicted, mitochondrial is mentioned; which is operable linked, too.

In another embodiment thereof the expression cassette does not comprise an additional transit peptide in case in Table I, application no. 12, in column 6 in the same line as SEQ ID NO. 115056 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 77774, or a homolog or fragment thereof, or a homolog thereof being depicted in Table I, application no. 12, column 8, in the same line as SEQ ID NO. 77774, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 77774 is depicted, plastidic is mentioned, or a mitochondrial transit peptide in case in Table I, application no. 12, in column 6 in the same line as SEQ ID NO. 77774 is depicted, mitochondrial is mentioned; which is operable linked, too.

In another embodiment thereof the expression cassette does not comprise an additional transit peptide in case in Table I, application no. 12, in column 6 in the same line as SEQ ID NO. 77774 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7947, or a homolog or fragment thereof, or a homolog thereof being depicted in Table I, application no. 12, column 8, in the same line as SEQ ID NO. 7947, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 7947 is depicted, plastidic is mentioned, or a mitochondrial transit peptide in case in Table I, application no. 12, in column 6 in the same line as SEQ ID NO. 7947 is depicted, mitochondrial is mentioned; which is operable linked, too.

In another embodiment thereof the expression cassette does not comprise an additional transit peptide in case in Table I, application no. 12, in column 6 in the same line as SEQ ID NO. 7947 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38300, or a homolog or fragment thereof, or a homolog thereof being depicted in Table I, application no. 12, column 8, in the same line as SEQ ID NO. 38300, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 38300 is depicted, plastidic is mentioned, or a mitochondrial transit peptide in case in Table I, application no. 12, in column 6 in the same line as SEQ ID NO. 38300 is depicted, mitochondrial is mentioned; which is operable linked, too.

In another embodiment thereof the expression cassette does not comprise an additional transit peptide in case in Table I, application no. 12, in column 6 in the same line as SEQ ID NO. 38300 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7992, or a homolog or fragment thereof, or a homolog thereof being depicted in Table I, application no. 12, column 8, in the same line as SEQ ID NO. 7992, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 7992 is depicted, plastidic is mentioned, or a mitochondrial transit peptide in case in Table I, application no. 12, in column 6 in the same line as SEQ ID NO. 7992 is depicted, mitochondrial is mentioned; which is operable linked, too.

In another embodiment thereof the expression cassette does not comprise an additional transit peptide in case in Table I, application no. 12, in column 6 in the same line as SEQ ID NO. 7992 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 124571, or a homolog or fragment thereof, or a homolog thereof being depicted in Table I, application no. 12, column 8, in the same line as SEQ ID NO. 124571, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 124571 is depicted, plastidic is mentioned, or a mitochondrial transit peptide in case in Table I, application no. 12, in column 6 in the same line as SEQ ID NO. 124571 is depicted, mitochondrial is mentioned; which is operable linked, too.

In another embodiment thereof the expression cassette does not comprise an additional transit peptide in case in Table I, application no. 12, in column 6 in the same line as SEQ ID NO. 124571 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38345, or a homolog or fragment thereof, or a homolog thereof being depicted in Table I, application no. 12, column 8, in the same line as SEQ ID NO. 38345, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 38345 is depicted, plastidic is mentioned, or a mitochondrial transit peptide in case in Table I, application no. 12, in column 6 in the same line as SEQ ID NO. 38345 is depicted, mitochondrial is mentioned; which is operable linked, too.

In another embodiment thereof the expression cassette does not comprise an additional transit peptide in case in Table I, application no. 12, in column 6 in the same line as SEQ ID NO. 38345 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 124805, or a homolog or fragment thereof, or a homolog thereof being depicted in Table I, application no. 12, column 8, in the same line as SEQ ID NO. 124805, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 124805 is depicted, plastidic is mentioned, or a mitochondrial transit peptide in case in Table I, application no. 12, in column 6 in the same line as SEQ ID NO. 124805 is depicted, mitochondrial is mentioned; which is operable linked, too.

In another embodiment thereof the expression cassette does not comprise an additional transit peptide in case in Table I, application no. 12, in column 6 in the same line as SEQ ID NO. 124805 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78753, or a homolog or fragment thereof, or a homolog thereof being depicted in Table I, application no. 12, column 8, in the same line as SEQ ID NO. 78753, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 78753 is depicted, plastidic is mentioned, or a mitochondrial transit peptide in case in Table I, application no. 12, in column 6 in the same line as SEQ ID NO. 78753 is depicted, mitochondrial is mentioned; which is operable linked, too.

In another embodiment thereof the expression cassette does not comprise an additional transit peptide in case in Table I, application no. 12, in column 6 in the same line as SEQ ID NO. 78753 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 124907, or a homolog or fragment thereof, or a homolog thereof being depicted in Table I, application no. 12, column 8, in the same line as SEQ ID NO. 124907, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 124907 is depicted, plastidic is mentioned, or a mitochondrial transit peptide in case in Table I, application no. 12, in column 6 in the same line as SEQ ID NO. 124907 is depicted, mitochondrial is mentioned; which is operable linked, too.

In another embodiment thereof the expression cassette does not comprise an additional transit peptide in case in Table I, application no. 12, in column 6 in the same line as SEQ ID NO. 124907 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78883, or a homolog or fragment thereof, or a homolog thereof being depicted in Table I, application no. 12, column 8, in the same line as SEQ ID NO. 78883, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 78883 is depicted, plastidic is mentioned, or a mitochondrial transit peptide in case in Table I, application no. 12, in column 6 in the same line as SEQ ID NO. 78883 is depicted, mitochondrial is mentioned; which is operable linked, too.

In another embodiment thereof the expression cassette does not comprise an additional transit peptide in case in Table I, application no. 12, in column 6 in the same line as SEQ ID NO. 78883 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78953, or a homolog or fragment thereof, or a homolog thereof being depicted in Table I, application no. 12, column 8, in the same line as SEQ ID NO. 78953, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 78953 is depicted, plastidic is mentioned, or a mitochondrial transit peptide in case in Table I, application no. 12, in column 6 in the same line as SEQ ID NO. 78953 is depicted, mitochondrial is mentioned; which is operable linked, too.

In another embodiment thereof the expression cassette does not comprise an additional transit peptide in case in Table I, application no. 12, in column 6 in the same line as SEQ ID NO. 78953 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 8920, or a homolog or fragment thereof, or a homolog thereof being depicted in Table I, application no. 12, column 8, in the same line as SEQ ID NO. 8920, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 8920 is depicted, plastidic is mentioned, or a mitochondrial transit peptide in case in Table I, application no. 12, in column 6 in the same line as SEQ ID NO. 8920 is depicted, mitochondrial is mentioned; which is operable linked, too.

In another embodiment thereof the expression cassette does not comprise an additional transit peptide in case in Table I, application no. 12, in column 6 in the same line as SEQ ID NO. 8920 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115595, or a homolog or fragment thereof, or a homolog thereof being depicted in Table I, application no. 12, column 8, in the same line as SEQ ID NO. 115595, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 115595 is depicted, plastidic is mentioned, or a mitochondrial transit peptide in case in Table I, application no. 12, in column 6 in the same line as SEQ ID NO. 115595 is depicted, mitochondrial is mentioned; which is operable linked, too.

In another embodiment thereof the expression cassette does not comprise an additional transit peptide in case in Table I, application no. 12, in column 6 in the same line as SEQ ID NO. 115595 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39002, or a homolog or fragment thereof, or a homolog thereof being depicted in Table I, application no. 12, column 8, in the same line as SEQ ID NO. 39002, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 39002 is depicted, plastidic is mentioned, or a mitochondrial transit peptide in case in Table I, application no. 12, in column 6 in the same line as SEQ ID NO. 39002 is depicted, mitochondrial is mentioned; which is operable linked, too.

In another embodiment thereof the expression cassette does not comprise an additional transit peptide in case in Table I, application no. 12, in column 6 in the same line as SEQ ID NO. 39002 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 124992, or a homolog or fragment thereof, or a homolog thereof being depicted in Table I, application no. 12, column 8, in the same line as SEQ ID NO. 124992, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 124992 is depicted, plastidic is mentioned, or a mitochondrial transit peptide in case in Table I, application no. 12, in column 6 in the same line as SEQ ID NO. 124992 is depicted, mitochondrial is mentioned; which is operable linked, too.

In another embodiment thereof the expression cassette does not comprise an additional transit peptide in case in Table I, application no. 12, in column 6 in the same line as SEQ ID NO. 124992 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 125085, or a homolog or fragment thereof, or a homolog thereof being depicted in Table I, application no. 12, column 8, in the same line as SEQ ID NO. 125085, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 125085 is depicted, plastidic is mentioned, or a mitochondrial transit peptide in case in Table I, application no. 12, in column 6 in the same line as SEQ ID NO. 125085 is depicted, mitochondrial is mentioned; which is operable linked, too.

In another embodiment thereof the expression cassette does not comprise an additional transit peptide in case in Table I, application no. 12, in column 6 in the same line as SEQ ID NO. 125085 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39040, or a homolog or fragment thereof, or a homolog thereof being depicted in Table I, application no. 12, column 8, in the same line as SEQ ID NO. 39040, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 39040 is depicted, plastidic is mentioned, or a mitochondrial transit peptide in case in Table I, application no. 12, in column 6 in the same line as SEQ ID NO. 39040 is depicted, mitochondrial is mentioned; which is operable linked, too.

In another embodiment thereof the expression cassette does not comprise an additional transit peptide in case in Table I, application no. 12, in column 6 in the same line as SEQ ID NO. 39040 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 125105, or a homolog or fragment thereof, or a homolog thereof being depicted in Table I, application no. 12, column 8, in the same line as SEQ ID NO. 125105, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 125105 is depicted, plastidic is mentioned, or a mitochondrial transit peptide in case in Table I, application no. 12, in column 6 in the same line as SEQ ID NO. 125105 is depicted, mitochondrial is mentioned; which is operable linked, too.

In another embodiment thereof the expression cassette does not comprise an additional transit peptide in case in Table I, application no. 12, in column 6 in the same line as SEQ ID NO. 125105 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39219, or a homolog or fragment thereof, or a homolog thereof being depicted in Table I, application no. 12, column 8, in the same line as SEQ ID NO. 39219, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 39219 is depicted, plastidic is mentioned, or a mitochondrial transit peptide in case in Table I, application no. 12, in column 6 in the same line as SEQ ID NO. 39219 is depicted, mitochondrial is mentioned; which is operable linked, too.

In another embodiment thereof the expression cassette does not comprise an additional transit peptide in case in Table I, application no. 12, in column 6 in the same line as SEQ ID NO. 39219 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39300, or a homolog or fragment thereof, or a homolog thereof being depicted in Table I, application no. 12, column 8, in the same line as SEQ ID NO. 39300, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 39300 is depicted, plastidic is mentioned, or a mitochondrial transit peptide in case in Table I, application no. 12, in column 6 in the same line as SEQ ID NO. 39300 is depicted, mitochondrial is mentioned; which is operable linked, too.

In another embodiment thereof the expression cassette does not comprise an additional transit peptide in case in Table I, application no. 12, in column 6 in the same line as SEQ ID NO. 39300 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115628, or a homolog or fragment thereof, or a homolog thereof being depicted in Table I, application no. 12, column 8, in the same line as SEQ ID NO. 115628, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 115628 is depicted, plastidic is mentioned, or a mitochondrial transit peptide in case in Table I, application no. 12, in column 6 in the same line as SEQ ID NO. 115628 is depicted, mitochondrial is mentioned; which is operable linked, too.

In another embodiment thereof the expression cassette does not comprise an additional transit peptide in case in Table I, application no. 12, in column 6 in the same line as SEQ ID NO. 115628 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40329, or a homolog or fragment thereof, or a homolog thereof being depicted in Table I, application no. 12, column 8, in the same line as SEQ ID NO. 40329, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 40329 is depicted, plastidic is mentioned, or a mitochondrial transit peptide in case in Table I, application no. 12, in column 6 in the same line as SEQ ID NO. 40329 is depicted, mitochondrial is mentioned; which is operable linked, too.

In another embodiment thereof the expression cassette does not comprise an additional transit peptide in case in Table I, application no. 12, in column 6 in the same line as SEQ ID NO. 40329 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 9167, or a homolog or fragment thereof, or a homolog thereof being depicted in Table I, application no. 12, column 8, in the same line as SEQ ID NO. 9167, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 9167 is depicted, plastidic is mentioned, or a mitochondrial transit peptide in case in Table I, application no. 12, in column 6 in the same line as SEQ ID NO. 9167 is depicted, mitochondrial is mentioned; which is operable linked, too.

In another embodiment thereof the expression cassette does not comprise an additional transit peptide in case in Table I, application no. 12, in column 6 in the same line as SEQ ID NO. 9167 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 125142, or a homolog or fragment thereof, or a homolog thereof being depicted in Table I, application no. 12, column 8, in the same line as SEQ ID NO. 125142, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 125142 is depicted, plastidic is mentioned, or a mitochondrial transit peptide in case in Table I, application no. 12, in column 6 in the same line as SEQ ID NO. 125142 is depicted, mitochondrial is mentioned; which is operable linked, too.

In another embodiment thereof the expression cassette does not comprise an additional transit peptide in case in Table I, application no. 12, in column 6 in the same line as SEQ ID NO. 125142 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 125226, or a homolog or fragment thereof, or a homolog thereof being depicted in Table I, application no. 12, column 8, in the same line as SEQ ID NO. 125226, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 125226 is depicted, plastidic is mentioned, or a mitochondrial transit peptide in case in Table I, application no. 12, in column 6 in the same line as SEQ ID NO. 125226 is depicted, mitochondrial is mentioned; which is operable linked, too.

In another embodiment thereof the expression cassette does not comprise an additional transit peptide in case in Table I, application no. 12, in column 6 in the same line as SEQ ID NO. 125226 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 81075, or a homolog or fragment thereof, or a homolog thereof being depicted in Table I, application no. 12, column 8, in the same line as SEQ ID NO. 81075, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 81075 is depicted, plastidic is mentioned, or a mitochondrial transit peptide in case in Table I, application no. 12, in column 6 in the same line as SEQ ID NO. 81075 is depicted, mitochondrial is mentioned; which is operable linked, too.

In another embodiment thereof the expression cassette does not comprise an additional transit peptide in case in Table I, application no. 12, in column 6 in the same line as SEQ ID NO. 81075 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 125234, or a homolog or fragment thereof, or a homolog thereof being depicted in Table I, application no. 12, column 8, in the same line as SEQ ID NO. 125234, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 125234 is depicted, plastidic is mentioned, or a mitochondrial transit peptide in case in Table I, application no. 12, in column 6 in the same line as SEQ ID NO. 125234 is depicted, mitochondrial is mentioned; which is operable linked, too.

In another embodiment thereof the expression cassette does not comprise an additional transit peptide in case in Table I, application no. 12, in column 6 in the same line as SEQ ID NO. 125234 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 41732, or a homolog or fragment thereof, or a homolog thereof being depicted in Table I, application no. 12, column 8, in the same line as SEQ ID NO. 41732, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 41732 is depicted, plastidic is mentioned, or a mitochondrial transit peptide in case in Table I, application no. 12, in column 6 in the same line as SEQ ID NO. 41732 is depicted, mitochondrial is mentioned; which is operable linked, too.

In another embodiment thereof the expression cassette does not comprise an additional transit peptide in case in Table I, application no. 12, in column 6 in the same line as SEQ ID NO. 41732 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 94380, or a homolog or fragment thereof, or a homolog thereof being depicted in Table I, application no. 12, column 8, in the same line as SEQ ID NO. 94380, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 94380 is depicted, plastidic is mentioned, or a mitochondrial transit peptide in case in Table I, application no. 12, in column 6 in the same line as SEQ ID NO. 94380 is depicted, mitochondrial is mentioned; which is operable linked, too.

In another embodiment thereof the expression cassette does not comprise an additional transit peptide in case in Table I, application no. 12, in column 6 in the same line as SEQ ID NO. 94380 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 125264, or a homolog or fragment thereof, or a homolog thereof being depicted in Table I, application no. 12, column 8, in the same line as SEQ ID NO. 125264, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 125264 is depicted, plastidic is mentioned, or a mitochondrial transit peptide in case in Table I, application no. 12, in column 6 in the same line as SEQ ID NO. 125264 is depicted, mitochondrial is mentioned; which is operable linked, too.

In another embodiment thereof the expression cassette does not comprise an additional transit peptide in case in Table I, application no. 12, in column 6 in the same line as SEQ ID NO. 125264 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 125487, or a homolog or fragment thereof, or a homolog thereof being depicted in Table I, application no. 12, column 8, in the same line as SEQ ID NO. 125487, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 125487 is depicted, plastidic is mentioned, or a mitochondrial transit peptide in case in Table I, application no. 12, in column 6 in the same line as SEQ ID NO. 125487 is depicted, mitochondrial is mentioned; which is operable linked, too.

In another embodiment thereof the expression cassette does not comprise an additional transit peptide in case in Table I, application no. 12, in column 6 in the same line as SEQ ID NO. 125487 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 100675, or a homolog or fragment thereof, or a homolog thereof being depicted in Table I, application no. 12, column 8, in the same line as SEQ ID NO. 100675, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 100675 is depicted, plastidic is mentioned, or a mitochondrial transit peptide in case in Table I, application no. 12, in column 6 in the same line as SEQ ID NO. 100675 is depicted, mitochondrial is mentioned; which is operable linked, too.

In another embodiment thereof the expression cassette does not comprise an additional transit peptide in case in Table I, application no. 12, in column 6 in the same line as SEQ ID NO. 100675 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 100675, or a homolog or fragment thereof, or a homolog thereof being depicted in Table I, application no. 12, column 8, in the same line as SEQ ID NO. 100675, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 100675 is depicted, plastidic is mentioned, or a mitochondrial transit peptide in case in Table I, application no. 12, in column 6 in the same line as SEQ ID NO. 100675 is depicted, mitochondrial is mentioned; which is operable linked, too.

In another embodiment thereof the expression cassette does not comprise an additional transit peptide in case in Table I, application no. 12, in column 6 in the same line as SEQ ID NO. 100675 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 83821, or a homolog or fragment thereof, or a homolog thereof being depicted in Table I, application no. 12, column 8, in the same line as SEQ ID NO. 83821, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 83821 is depicted, plastidic is mentioned, or a mitochondrial transit peptide in case in Table I, application no. 12, in column 6 in the same line as SEQ ID NO. 83821 is depicted, mitochondrial is mentioned; which is operable linked, too.

In another embodiment thereof the expression cassette does not comprise an additional transit peptide in case in Table I, application no. 12, in column 6 in the same line as SEQ ID NO. 83821 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 125541, or a homolog or fragment thereof, or a homolog thereof being depicted in Table I, application no. 12, column 8, in the same line as SEQ ID NO. 125541, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 125541 is depicted, plastidic is mentioned, or a mitochondrial transit peptide in case in Table I, application no. 12, in column 6 in the same line as SEQ ID NO. 125541 is depicted, mitochondrial is mentioned; which is operable linked, too.

In another embodiment thereof the expression cassette does not comprise an additional transit peptide in case in Table I, application no. 12, in column 6 in the same line as SEQ ID NO. 125541 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 125795, or a homolog or fragment thereof, or a homolog thereof being depicted in Table I, application no. 12, column 8, in the same line as SEQ ID NO. 125795, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 125795 is depicted, plastidic is mentioned, or a mitochondrial transit peptide in case in Table I, application no. 12, in column 6 in the same line as SEQ ID NO. 125795 is depicted, mitochondrial is mentioned; which is operable linked, too.

In another embodiment thereof the expression cassette does not comprise an additional transit peptide in case in Table I, application no. 12, in column 6 in the same line as SEQ ID NO. 125795 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 126377, or a homolog or fragment thereof, or a homolog thereof being depicted in Table I, application no. 12, column 8, in the same line as SEQ ID NO. 126377, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 126377 is depicted, plastidic is mentioned, or a mitochondrial transit peptide in case in Table I, application no. 12, in column 6 in the same line as SEQ ID NO. 126377 is depicted, mitochondrial is mentioned; which is operable linked, too.

In another embodiment thereof the expression cassette does not comprise an additional transit peptide in case in Table I, application no. 12, in column 6 in the same line as SEQ ID NO. 126377 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 126415, or a homolog or fragment thereof, or a homolog thereof being depicted in Table I, application no. 12, column 8, in the same line as SEQ ID NO. 126415, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 126415 is depicted, plastidic is mentioned, or a mitochondrial transit peptide in case in Table I, application no. 12, in column 6 in the same line as SEQ ID NO. 126415 is depicted, mitochondrial is mentioned; which is operable linked, too.

In another embodiment thereof the expression cassette does not comprise an additional transit peptide in case in Table I, application no. 12, in column 6 in the same line as SEQ ID NO. 126415 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 119209, or a homolog or fragment thereof, or a homolog thereof being depicted in Table I, application no. 12, column 8, in the same line as SEQ ID NO. 119209, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 119209 is depicted, plastidic is mentioned, or a mitochondrial transit peptide in case in Table I, application no. 12, in column 6 in the same line as SEQ ID NO. 119209 is depicted, mitochondrial is mentioned; which is operable linked, too.

In another embodiment thereof the expression cassette does not comprise an additional transit peptide in case in Table I, application no. 12, in column 6 in the same line as SEQ ID NO. 119209 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 126421, or a homolog or fragment thereof, or a homolog thereof being depicted in Table I, application no. 12, column 8, in the same line as SEQ ID NO. 126421, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 126421 is depicted, plastidic is mentioned, or a mitochondrial transit peptide in case in Table I, application no. 12, in column 6 in the same line as SEQ ID NO. 126421 is depicted, mitochondrial is mentioned; which is operable linked, too.

In another embodiment thereof the expression cassette does not comprise an additional transit peptide in case in Table I, application no. 12, in column 6 in the same line as SEQ ID NO. 126421 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 126975, or a homolog or fragment thereof, or a homolog thereof being depicted in Table I, application no. 12, column 8, in the same line as SEQ ID NO. 126975, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 126975 is depicted, plastidic is mentioned, or a mitochondrial transit peptide in case in Table I, application no. 12, in column 6 in the same line as SEQ ID NO. 126975 is depicted, mitochondrial is mentioned; which is operable linked, too.

In another embodiment thereof the expression cassette does not comprise an additional transit peptide in case in Table I, application no. 12, in column 6 in the same line as SEQ ID NO. 126975 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 126991, or a homolog or fragment thereof, or a homolog thereof being depicted in Table I, application no. 12, column 8, in the same line as SEQ ID NO. 126991, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 126991 is depicted, plastidic is mentioned, or a mitochondrial transit peptide in case in Table I, application no. 12, in column 6 in the same line as SEQ ID NO. 126991 is depicted, mitochondrial is mentioned; which is operable linked, too.

In another embodiment thereof the expression cassette does not comprise an additional transit peptide in case in Table I, application no. 12, in column 6 in the same line as SEQ ID NO. 126991 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 10811, or a homolog or fragment thereof, or a homolog thereof being depicted in Table I, application no. 12, column 8, in the same line as SEQ ID NO. 10811, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 10811 is depicted, plastidic is mentioned, or a mitochondrial transit peptide in case in Table I, application no. 12, in column 6 in the same line as SEQ ID NO. 10811 is depicted, mitochondrial is mentioned; which is operable linked, too.

In another embodiment thereof the expression cassette does not comprise an additional transit peptide in case in Table I, application no. 12, in column 6 in the same line as SEQ ID NO. 10811 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 46751, or a homolog or fragment thereof, or a homolog thereof being depicted in Table I, application no. 12, column 8, in the same line as SEQ ID NO. 46751, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 46751 is depicted, plastidic is mentioned, or a mitochondrial transit peptide in case in Table I, application no. 12, in column 6 in the same line as SEQ ID NO. 46751 is depicted, mitochondrial is mentioned; which is operable linked, too.

In another embodiment thereof the expression cassette does not comprise an additional transit peptide in case in Table I, application no. 12, in column 6 in the same line as SEQ ID NO. 46751 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 116312, or a homolog or fragment thereof, or a homolog thereof being depicted in Table I, application no. 12, column 8, in the same line as SEQ ID NO. 116312, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 116312 is depicted, plastidic is mentioned, or a mitochondrial transit peptide in case in Table I, application no. 12, in column 6 in the same line as SEQ ID NO. 116312 is depicted, mitochondrial is mentioned; which is operable linked, too.

In another embodiment thereof the expression cassette does not comprise an additional transit peptide in case in Table I, application no. 12, in column 6 in the same line as SEQ ID NO. 116312 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 84865, or a homolog or fragment thereof, or a homolog thereof being depicted in Table I, application no. 12, column 8, in the same line as SEQ ID NO. 84865, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 84865 is depicted, plastidic is mentioned, or a mitochondrial transit peptide in case in Table I, application no. 12, in column 6 in the same line as SEQ ID NO. 84865 is depicted, mitochondrial is mentioned; which is operable linked, too.

In another embodiment thereof the expression cassette does not comprise an additional transit peptide in case in Table I, application no. 12, in column 6 in the same line as SEQ ID NO. 84865 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 106920, or a homolog or fragment thereof, or a homolog thereof being depicted in Table I, application no. 12, column 8, in the same line as SEQ ID NO. 106920, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 106920 is depicted, plastidic is mentioned, or a mitochondrial transit peptide in case in Table I, application no. 12, in column 6 in the same line as SEQ ID NO. 106920 is depicted, mitochondrial is mentioned; which is operable linked, too.

In another embodiment thereof the expression cassette does not comprise an additional transit peptide in case in Table I, application no. 12, in column 6 in the same line as SEQ ID NO. 106920 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 127103, or a homolog or fragment thereof, or a homolog thereof being depicted in Table I, application no. 12, column 8, in the same line as SEQ ID NO. 127103, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 127103 is depicted, plastidic is mentioned, or a mitochondrial transit peptide in case in Table I, application no. 12, in column 6 in the same line as SEQ ID NO. 127103 is depicted, mitochondrial is mentioned; which is operable linked, too.

In another embodiment thereof the expression cassette does not comprise an additional transit peptide in case in Table I, application no. 12, in column 6 in the same line as SEQ ID NO. 127103 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 101707, or a homolog or fragment thereof, or a homolog thereof being depicted in Table I, application no. 12, column 8, in the same line as SEQ ID NO. 101707, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 101707 is depicted, plastidic is mentioned, or a mitochondrial transit peptide in case in Table I, application no. 12, in column 6 in the same line as SEQ ID NO. 101707 is depicted, mitochondrial is mentioned; which is operable linked, too.

In another embodiment thereof the expression cassette does not comprise an additional transit peptide in case in Table I, application no. 12, in column 6 in the same line as SEQ ID NO. 101707 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 127109, or a homolog or fragment thereof, or a homolog thereof being depicted in Table I, application no. 12, column 8, in the same line as SEQ ID NO. 127109, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 127109 is depicted, plastidic is mentioned, or a mitochondrial transit peptide in case in Table I, application no. 12, in column 6 in the same line as SEQ ID NO. 127109 is depicted, mitochondrial is mentioned; which is operable linked, too.

In another embodiment thereof the expression cassette does not comprise an additional transit peptide in case in Table I, application no. 12, in column 6 in the same line as SEQ ID NO. 127109 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 52634, or a homolog or fragment thereof, or a homolog thereof being depicted in Table I, application no. 12, column 8, in the same line as SEQ ID NO. 52634, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 52634 is depicted, plastidic is mentioned, or a mitochondrial transit peptide in case in Table I, application no. 12, in column 6 in the same line as SEQ ID NO. 52634 is depicted, mitochondrial is mentioned; which is operable linked, too.

In another embodiment thereof the expression cassette does not comprise an additional transit peptide in case in Table I, application no. 12, in column 6 in the same line as SEQ ID NO. 52634 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 11471, or a homolog or fragment thereof, or a homolog thereof being depicted in Table I, application no. 12, column 8, in the same line as SEQ ID NO. 11471, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 11471 is depicted, plastidic is mentioned, or a mitochondrial transit peptide in case in Table I, application no. 12, in column 6 in the same line as SEQ ID NO. 11471 is depicted, mitochondrial is mentioned; which is operable linked, too.

In another embodiment thereof the expression cassette does not comprise an additional transit peptide in case in Table I, application no. 12, in column 6 in the same line as SEQ ID NO. 11471 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 11990, or a homolog or fragment thereof, or a homolog thereof being depicted in Table I, application no. 12, column 8, in the same line as SEQ ID NO. 11990, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 11990 is depicted, plastidic is mentioned, or a mitochondrial transit peptide in case in Table I, application no. 12, in column 6 in the same line as SEQ ID NO. 11990 is depicted, mitochondrial is mentioned; which is operable linked, too.

In another embodiment thereof the expression cassette does not comprise an additional transit peptide in case in Table I, application no. 12, in column 6 in the same line as SEQ ID NO. 11990 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 56153, or a homolog or fragment thereof, or a homolog thereof being depicted in Table I, application no. 12, column 8, in the same line as SEQ ID NO. 56153, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 56153 is depicted, plastidic is mentioned, or a mitochondrial transit peptide in case in Table I, application no. 12, in column 6 in the same line as SEQ ID NO. 56153 is depicted, mitochondrial is mentioned; which is operable linked, too.

In another embodiment thereof the expression cassette does not comprise an additional transit peptide in case in Table I, application no. 12, in column 6 in the same line as SEQ ID NO. 56153 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 87397, or a homolog or fragment thereof, or a homolog thereof being depicted in Table I, application no. 12, column 8, in the same line as SEQ ID NO. 87397, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 87397 is depicted, plastidic is mentioned, or a mitochondrial transit peptide in case in Table I, application no. 12, in column 6 in the same line as SEQ ID NO. 87397 is depicted, mitochondrial is mentioned; which is operable linked, too.

In another embodiment thereof the expression cassette does not comprise an additional transit peptide in case in Table I, application no. 12, in column 6 in the same line as SEQ ID NO. 87397 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 127183, or a homolog or fragment thereof, or a homolog thereof being depicted in Table I, application no. 12, column 8, in the same line as SEQ ID NO. 127183, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 127183 is depicted, plastidic is mentioned, or a mitochondrial transit peptide in case in Table I, application no. 12, in column 6 in the same line as SEQ ID NO. 127183 is depicted, mitochondrial is mentioned; which is operable linked, too.

In another embodiment thereof the expression cassette does not comprise an additional transit peptide in case in Table I, application no. 12, in column 6 in the same line as SEQ ID NO. 127183 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 119222, or a homolog or fragment thereof, or a homolog thereof being depicted in Table I, application no. 12, column 8, in the same line as SEQ ID NO. 119222, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 119222 is depicted, plastidic is mentioned, or a mitochondrial transit peptide in case in Table I, application no. 12, in column 6 in the same line as SEQ ID NO. 119222 is depicted, mitochondrial is mentioned; which is operable linked, too.

In another embodiment thereof the expression cassette does not comprise an additional transit peptide in case in Table I, application no. 12, in column 6 in the same line as SEQ ID NO. 119222 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 87655, or a homolog or fragment thereof, or a homolog thereof being depicted in Table I, application no. 12, column 8, in the same line as SEQ ID NO. 87655, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 87655 is depicted, plastidic is mentioned, or a mitochondrial transit peptide in case in Table I, application no. 12, in column 6 in the same line as SEQ ID NO. 87655 is depicted, mitochondrial is mentioned; which is operable linked, too.

In another embodiment thereof the expression cassette does not comprise an additional transit peptide in case in Table I, application no. 12, in column 6 in the same line as SEQ ID NO. 87655 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 57734, or a homolog or fragment thereof, or a homolog thereof being depicted in Table I, application no. 12, column 8, in the same line as SEQ ID NO. 57734, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 57734 is depicted, plastidic is mentioned, or a mitochondrial transit peptide in case in Table I, application no. 12, in column 6 in the same line as SEQ ID NO. 57734 is depicted, mitochondrial is mentioned; which is operable linked, too.

In another embodiment thereof the expression cassette does not comprise an additional transit peptide in case in Table I, application no. 12, in column 6 in the same line as SEQ ID NO. 57734 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 116460, or a homolog or fragment thereof, or a homolog thereof being depicted in Table I, application no. 12, column 8, in the same line as SEQ ID NO. 116460, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 116460 is depicted, plastidic is mentioned, or a mitochondrial transit peptide in case in Table I, application no. 12, in column 6 in the same line as SEQ ID NO. 116460 is depicted, mitochondrial is mentioned; which is operable linked, too.

In another embodiment thereof the expression cassette does not comprise an additional transit peptide in case in Table I, application no. 12, in column 6 in the same line as SEQ ID NO. 116460 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 127521, or a homolog or fragment thereof, or a homolog thereof being depicted in Table I, application no. 12, column 8, in the same line as SEQ ID NO. 127521, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 127521 is depicted, plastidic is mentioned, or a mitochondrial transit peptide in case in Table I, application no. 12, in column 6 in the same line as SEQ ID NO. 127521 is depicted, mitochondrial is mentioned; which is operable linked, too.

In another embodiment thereof the expression cassette does not comprise an additional transit peptide in case in Table I, application no. 12, in column 6 in the same line as SEQ ID NO. 127521 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12341, or a homolog or fragment thereof, or a homolog thereof being depicted in Table I, application no. 12, column 8, in the same line as SEQ ID NO. 12341, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 12341 is depicted, plastidic is mentioned, or a mitochondrial transit peptide in case in Table I, application no. 12, in column 6 in the same line as SEQ ID NO. 12341 is depicted, mitochondrial is mentioned; which is operable linked, too.

In another embodiment thereof the expression cassette does not comprise an additional transit peptide in case in Table I, application no. 12, in column 6 in the same line as SEQ ID NO. 12341 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 128024, or a homolog or fragment thereof, or a homolog thereof being depicted in Table I, application no. 12, column 8, in the same line as SEQ ID NO. 128024, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 128024 is depicted, plastidic is mentioned, or a mitochondrial transit peptide in case in Table I, application no. 12, in column 6 in the same line as SEQ ID NO. 128024 is depicted, mitochondrial is mentioned; which is operable linked, too.

In another embodiment thereof the expression cassette does not comprise an additional transit peptide in case in Table I, application no. 12, in column 6 in the same line as SEQ ID NO. 128024 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12698, or a homolog or fragment thereof, or a homolog thereof being depicted in Table I, application no. 12, column 8, in the same line as SEQ ID NO. 12698, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 12698 is depicted, plastidic is mentioned, or a mitochondrial transit peptide in case in Table I, application no. 12, in column 6 in the same line as SEQ ID NO. 12698 is depicted, mitochondrial is mentioned; which is operable linked, too.

In another embodiment thereof the expression cassette does not comprise an additional transit peptide in case in Table I, application no. 12, in column 6 in the same line as SEQ ID NO. 12698 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 61070, or a homolog or fragment thereof, or a homolog thereof being depicted in Table I, application no. 12, column 8, in the same line as SEQ ID NO. 61070, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 61070 is depicted, plastidic is mentioned, or a mitochondrial transit peptide in case in Table I, application no. 12, in column 6 in the same line as SEQ ID NO. 61070 is depicted, mitochondrial is mentioned; which is operable linked, too.

In another embodiment thereof the expression cassette does not comprise an additional transit peptide in case in Table I, application no. 12, in column 6 in the same line as SEQ ID NO. 61070 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 61553, or a homolog or fragment thereof, or a homolog thereof being depicted in Table I, application no. 12, column 8, in the same line as SEQ ID NO. 61553, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 61553 is depicted, plastidic is mentioned, or a mitochondrial transit peptide in case in Table I, application no. 12, in column 6 in the same line as SEQ ID NO. 61553 is depicted, mitochondrial is mentioned; which is operable linked, too.

In another embodiment thereof the expression cassette does not comprise an additional transit peptide in case in Table I, application no. 12, in column 6 in the same line as SEQ ID NO. 61553 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 61723, or a homolog or fragment thereof, or a homolog thereof being depicted in Table I, application no. 12, column 8, in the same line as SEQ ID NO. 61723, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 61723 is depicted, plastidic is mentioned, or a mitochondrial transit peptide in case in Table I, application no. 12, in column 6 in the same line as SEQ ID NO. 61723 is depicted, mitochondrial is mentioned; which is operable linked, too.

In another embodiment thereof the expression cassette does not comprise an additional transit peptide in case in Table I, application no. 12, in column 6 in the same line as SEQ ID NO. 61723 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 62079, or a homolog or fragment thereof, or a homolog thereof being depicted in Table I, application no. 12, column 8, in the same line as SEQ ID NO. 62079, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 62079 is depicted, plastidic is mentioned, or a mitochondrial transit peptide in case in Table I, application no. 12, in column 6 in the same line as SEQ ID NO. 62079 is depicted, mitochondrial is mentioned; which is operable linked, too.

In another embodiment thereof the expression cassette does not comprise an additional transit peptide in case in Table I, application no. 12, in column 6 in the same line as SEQ ID NO. 62079 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12974, or a homolog or fragment thereof, or a homolog thereof being depicted in Table I, application no. 12, column 8, in the same line as SEQ ID NO. 12974, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 12974 is depicted, plastidic is mentioned, or a mitochondrial transit peptide in case in Table I, application no. 12, in column 6 in the same line as SEQ ID NO. 12974 is depicted, mitochondrial is mentioned; which is operable linked, too.

In another embodiment thereof the expression cassette does not comprise an additional transit peptide in case in Table I, application no. 12, in column 6 in the same line as SEQ ID NO. 12974 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 128139, or a homolog or fragment thereof, or a homolog thereof being depicted in Table I, application no. 12, column 8, in the same line as SEQ ID NO. 128139, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 128139 is depicted, plastidic is mentioned, or a mitochondrial transit peptide in case in Table I, application no. 12, in column 6 in the same line as SEQ ID NO. 128139 is depicted, mitochondrial is mentioned; which is operable linked, too.

In another embodiment thereof the expression cassette does not comprise an additional transit peptide in case in Table I, application no. 12, in column 6 in the same line as SEQ ID NO. 128139 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 62524, or a homolog or fragment thereof, or a homolog thereof being depicted in Table I, application no. 12, column 8, in the same line as SEQ ID NO. 62524, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 62524 is depicted, plastidic is mentioned, or a mitochondrial transit peptide in case in Table I, application no. 12, in column 6 in the same line as SEQ ID NO. 62524 is depicted, mitochondrial is mentioned; which is operable linked, too.

In another embodiment thereof the expression cassette does not comprise an additional transit peptide in case in Table I, application no. 12, in column 6 in the same line as SEQ ID NO. 62524 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 62717, or a homolog or fragment thereof, or a homolog thereof being depicted in Table I, application no. 12, column 8, in the same line as SEQ ID NO. 62717, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 62717 is depicted, plastidic is mentioned, or a mitochondrial transit peptide in case in Table I, application no. 12, in column 6 in the same line as SEQ ID NO. 62717 is depicted, mitochondrial is mentioned; which is operable linked, too.

In another embodiment thereof the expression cassette does not comprise an additional transit peptide in case in Table I, application no. 12, in column 6 in the same line as SEQ ID NO. 62717 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 96980, or a homolog or fragment thereof, or a homolog thereof being depicted in Table I, application no. 12, column 8, in the same line as SEQ ID NO. 96980, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 96980 is depicted, plastidic is mentioned, or a mitochondrial transit peptide in case in Table I, application no. 12, in column 6 in the same line as SEQ ID NO. 96980 is depicted, mitochondrial is mentioned; which is operable linked, too.

In another embodiment thereof the expression cassette does not comprise an additional transit peptide in case in Table I, application no. 12, in column 6 in the same line as SEQ ID NO. 96980 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 113220, or a homolog or fragment thereof, or a homolog thereof being depicted in Table I, application no. 12, column 8, in the same line as SEQ ID NO. 113220, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 113220 is depicted, plastidic is mentioned, or a mitochondrial transit peptide in case in Table I, application no. 12, in column 6 in the same line as SEQ ID NO. 113220 is depicted, mitochondrial is mentioned; which is operable linked, too.

In another embodiment thereof the expression cassette does not comprise an additional transit peptide in case in Table I, application no. 12, in column 6 in the same line as SEQ ID NO. 113220 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 119408, or a homolog or fragment thereof, or a homolog thereof being depicted in Table I, application no. 12, column 8, in the same line as SEQ ID NO. 119408, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 119408 is depicted, plastidic is mentioned, or a mitochondrial transit peptide in case in Table I, application no. 12, in column 6 in the same line as SEQ ID NO. 119408 is depicted, mitochondrial is mentioned; which is operable linked, too.

In another embodiment thereof the expression cassette does not comprise an additional transit peptide in case in Table I, application no. 12, in column 6 in the same line as SEQ ID NO. 119408 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 122228, or a homolog or fragment thereof, or a homolog thereof being depicted in Table I, application no. 12, column 8, in the same line as SEQ ID NO. 122228, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 122228 is depicted, plastidic is mentioned, or a mitochondrial transit peptide in case in Table I, application no. 12, in column 6 in the same line as SEQ ID NO. 122228 is depicted, mitochondrial is mentioned; which is operable linked, too.

In another embodiment thereof the expression cassette does not comprise an additional transit peptide in case in Table I, application no. 12, in column 6 in the same line as SEQ ID NO. 122228 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 120045, or a homolog or fragment thereof, or a homolog thereof being depicted in Table I, application no. 12, column 8, in the same line as SEQ ID NO. 120045, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 120045 is depicted, plastidic is mentioned, or a mitochondrial transit peptide in case in Table I, application no. 12, in column 6 in the same line as SEQ ID NO. 120045 is depicted, mitochondrial is mentioned; which is operable linked, too.

In another embodiment thereof the expression cassette does not comprise an additional transit peptide in case in Table I, application no. 12, in column 6 in the same line as SEQ ID NO. 120045 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 113224, or a homolog or fragment thereof, or a homolog thereof being depicted in Table I, application no. 12, column 8, in the same line as SEQ ID NO. 113224, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 113224 is depicted, plastidic is mentioned, or a mitochondrial transit peptide in case in Table I, application no. 12, in column 6 in the same line as SEQ ID NO. 113224 is depicted, mitochondrial is mentioned; which is operable linked, too.

In another embodiment thereof the expression cassette does not comprise an additional transit peptide in case in Table I, application no. 12, in column 6 in the same line as SEQ ID NO. 113224 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 113330, or a homolog or fragment thereof, or a homolog thereof being depicted in Table I, application no. 12, column 8, in the same line as SEQ ID NO. 113330, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 113330 is depicted, plastidic is mentioned, or a mitochondrial transit peptide in case in Table I, application no. 12, in column 6 in the same line as SEQ ID NO. 113330 is depicted, mitochondrial is mentioned; which is operable linked, too.

In another embodiment thereof the expression cassette does not comprise an additional transit peptide in case in Table I, application no. 12, in column 6 in the same line as SEQ ID NO. 113330 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 128148, or a homolog or fragment thereof, or a homolog thereof being depicted in Table I, application no. 12, column 8, in the same line as SEQ ID NO. 128148, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 128148 is depicted, plastidic is mentioned, or a mitochondrial transit peptide in case in Table I, application no. 12, in column 6 in the same line as SEQ ID NO. 128148 is depicted, mitochondrial is mentioned; which is operable linked, too.

In another embodiment thereof the expression cassette does not comprise an additional transit peptide in case in Table I, application no. 12, in column 6 in the same line as SEQ ID NO. 128148 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 14706, or a homolog or fragment thereof, or a homolog thereof being depicted in Table I, application no. 12, column 8, in the same line as SEQ ID NO. 14706, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 14706 is depicted, plastidic is mentioned, or a mitochondrial transit peptide in case in Table I, application no. 12, in column 6 in the same line as SEQ ID NO. 14706 is depicted, mitochondrial is mentioned; which is operable linked, too.

In another embodiment thereof the expression cassette does not comprise an additional transit peptide in case in Table I, application no. 12, in column 6 in the same line as SEQ ID NO. 14706 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 90159, or a homolog or fragment thereof, or a homolog thereof being depicted in Table I, application no. 12, column 8, in the same line as SEQ ID NO. 90159, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 90159 is depicted, plastidic is mentioned, or a mitochondrial transit peptide in case in Table I, application no. 12, in column 6 in the same line as SEQ ID NO. 90159 is depicted, mitochondrial is mentioned; which is operable linked, too.

In another embodiment thereof the expression cassette does not comprise an additional transit peptide in case in Table I, application no. 12, in column 6 in the same line as SEQ ID NO. 90159 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 120241, or a homolog or fragment thereof, or a homolog thereof being depicted in Table I, application no. 12, column 8, in the same line as SEQ ID NO. 120241, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 120241 is depicted, plastidic is mentioned, or a mitochondrial transit peptide in case in Table I, application no. 12, in column 6 in the same line as SEQ ID NO. 120241 is depicted, mitochondrial is mentioned; which is operable linked, too.

In another embodiment thereof the expression cassette does not comprise an additional transit peptide in case in Table I, application no. 12, in column 6 in the same line as SEQ ID NO. 120241 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 128511, or a homolog or fragment thereof, or a homolog thereof being depicted in Table I, application no. 12, column 8, in the same line as SEQ ID NO. 128511, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 128511 is depicted, plastidic is mentioned, or a mitochondrial transit peptide in case in Table I, application no. 12, in column 6 in the same line as SEQ ID NO. 128511 is depicted, mitochondrial is mentioned; which is operable linked, too.

In another embodiment thereof the expression cassette does not comprise an additional transit peptide in case in Table I, application no. 12, in column 6 in the same line as SEQ ID NO. 128511 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 113598, or a homolog or fragment thereof, or a homolog thereof being depicted in Table I, application no. 12, column 8, in the same line as SEQ ID NO. 113598, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 113598 is depicted, plastidic is mentioned, or a mitochondrial transit peptide in case in Table I, application no. 12, in column 6 in the same line as SEQ ID NO. 113598 is depicted, mitochondrial is mentioned; which is operable linked, too.

In another embodiment thereof the expression cassette does not comprise an additional transit peptide in case in Table I, application no. 12, in column 6 in the same line as SEQ ID NO. 113598 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 63807, or a homolog or fragment thereof, or a homolog thereof being depicted in Table I, application no. 12, column 8, in the same line as SEQ ID NO. 63807, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 63807 is depicted, plastidic is mentioned, or a mitochondrial transit peptide in case in Table I, application no. 12, in column 6 in the same line as SEQ ID NO. 63807 is depicted, mitochondrial is mentioned; which is operable linked, too.

In another embodiment thereof the expression cassette does not comprise an additional transit peptide in case in Table I, application no. 12, in column 6 in the same line as SEQ ID NO. 63807 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 64144, or a homolog or fragment thereof, or a homolog thereof being depicted in Table I, application no. 12, column 8, in the same line as SEQ ID NO. 64144, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 64144 is depicted, plastidic is mentioned, or a mitochondrial transit peptide in case in Table I, application no. 12, in column 6 in the same line as SEQ ID NO. 64144 is depicted, mitochondrial is mentioned; which is operable linked, too.

In another embodiment thereof the expression cassette does not comprise an additional transit peptide in case in Table I, application no. 12, in column 6 in the same line as SEQ ID NO. 64144 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 90189, or a homolog or fragment thereof, or a homolog thereof being depicted in Table I, application no. 12, column 8, in the same line as SEQ ID NO. 90189, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 90189 is depicted, plastidic is mentioned, or a mitochondrial transit peptide in case in Table I, application no. 12, in column 6 in the same line as SEQ ID NO. 90189 is depicted, mitochondrial is mentioned; which is operable linked, too.

In another embodiment thereof the expression cassette does not comprise an additional transit peptide in case in Table I, application no. 12, in column 6 in the same line as SEQ ID NO. 90189 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 64177, or a homolog or fragment thereof, or a homolog thereof being depicted in Table I, application no. 12, column 8, in the same line as SEQ ID NO. 64177, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 64177 is depicted, plastidic is mentioned, or a mitochondrial transit peptide in case in Table I, application no. 12, in column 6 in the same line as SEQ ID NO. 64177 is depicted, mitochondrial is mentioned; which is operable linked, too.

In another embodiment thereof the expression cassette does not comprise an additional transit peptide in case in Table I, application no. 12, in column 6 in the same line as SEQ ID NO. 64177 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 117482, or a homolog or fragment thereof, or a homolog thereof being depicted in Table I, application no. 12, column 8, in the same line as SEQ ID NO. 117482, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 117482 is depicted, plastidic is mentioned, or a mitochondrial transit peptide in case in Table I, application no. 12, in column 6 in the same line as SEQ ID NO. 117482 is depicted, mitochondrial is mentioned; which is operable linked, too.

In another embodiment thereof the expression cassette does not comprise an additional transit peptide in case in Table I, application no. 12, in column 6 in the same line as SEQ ID NO. 117482 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 64218, or a homolog or fragment thereof, or a homolog thereof being depicted in Table I, application no. 12, column 8, in the same line as SEQ ID NO. 64218, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 64218 is depicted, plastidic is mentioned, or a mitochondrial transit peptide in case in Table I, application no. 12, in column 6 in the same line as SEQ ID NO. 64218 is depicted, mitochondrial is mentioned; which is operable linked, too.

In another embodiment thereof the expression cassette does not comprise an additional transit peptide in case in Table I, application no. 12, in column 6 in the same line as SEQ ID NO. 64218 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 128655, or a homolog or fragment thereof, or a homolog thereof being depicted in Table I, application no. 12, column 8, in the same line as SEQ ID NO. 128655, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 128655 is depicted, plastidic is mentioned, or a mitochondrial transit peptide in case in Table I, application no. 12, in column 6 in the same line as SEQ ID NO. 128655 is depicted, mitochondrial is mentioned; which is operable linked, too.

In another embodiment thereof the expression cassette does not comprise an additional transit peptide in case in Table I, application no. 12, in column 6 in the same line as SEQ ID NO. 128655 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 128967, or a homolog or fragment thereof, or a homolog thereof being depicted in Table I, application no. 12, column 8, in the same line as SEQ ID NO. 128967, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 128967 is depicted, plastidic is mentioned, or a mitochondrial transit peptide in case in Table I, application no. 12, in column 6 in the same line as SEQ ID NO. 128967 is depicted, mitochondrial is mentioned; which is operable linked, too.

In another embodiment thereof the expression cassette does not comprise an additional transit peptide in case in Table I, application no. 12, in column 6 in the same line as SEQ ID NO. 128967 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 64964, or a homolog or fragment thereof, or a homolog thereof being depicted in Table I, application no. 12, column 8, in the same line as SEQ ID NO. 64964, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 64964 is depicted, plastidic is mentioned, or a mitochondrial transit peptide in case in Table I, application no. 12, in column 6 in the same line as SEQ ID NO. 64964 is depicted, mitochondrial is mentioned; which is operable linked, too.

In another embodiment thereof the expression cassette does not comprise an additional transit peptide in case in Table I, application no. 12, in column 6 in the same line as SEQ ID NO. 64964 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 97631, or a homolog or fragment thereof, or a homolog thereof being depicted in Table I, application no. 12, column 8, in the same line as SEQ ID NO. 97631, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 97631 is depicted, plastidic is mentioned, or a mitochondrial transit peptide in case in Table I, application no. 12, in column 6 in the same line as SEQ ID NO. 97631 is depicted, mitochondrial is mentioned; which is operable linked, too.

In another embodiment thereof the expression cassette does not comprise an additional transit peptide in case in Table I, application no. 12, in column 6 in the same line as SEQ ID NO. 97631 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 14843, or a homolog or fragment thereof, or a homolog thereof being depicted in Table I, application no. 12, column 8, in the same line as SEQ ID NO. 14843, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 14843 is depicted, plastidic is mentioned, or a mitochondrial transit peptide in case in Table I, application no. 12, in column 6 in the same line as SEQ ID NO. 14843 is depicted, mitochondrial is mentioned; which is operable linked, too.

In another embodiment thereof the expression cassette does not comprise an additional transit peptide in case in Table I, application no. 12, in column 6 in the same line as SEQ ID NO. 14843 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 66274, or a homolog or fragment thereof, or a homolog thereof being depicted in Table I, application no. 12, column 8, in the same line as SEQ ID NO. 66274, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 66274 is depicted, plastidic is mentioned, or a mitochondrial transit peptide in case in Table I, application no. 12, in column 6 in the same line as SEQ ID NO. 66274 is depicted, mitochondrial is mentioned; which is operable linked, too.

In another embodiment thereof the expression cassette does not comprise an additional transit peptide in case in Table I, application no. 12, in column 6 in the same line as SEQ ID NO. 66274 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 129065, or a homolog or fragment thereof, or a homolog thereof being depicted in Table I, application no. 12, column 8, in the same line as SEQ ID NO. 129065, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 129065 is depicted, plastidic is mentioned, or a mitochondrial transit peptide in case in Table I, application no. 12, in column 6 in the same line as SEQ ID NO. 129065 is depicted, mitochondrial is mentioned; which is operable linked, too.

In another embodiment thereof the expression cassette does not comprise an additional transit peptide in case in Table I, application no. 12, in column 6 in the same line as SEQ ID NO. 129065 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 129123, or a homolog or fragment thereof, or a homolog thereof being depicted in Table I, application no. 12, column 8, in the same line as SEQ ID NO. 129123, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 129123 is depicted, plastidic is mentioned, or a mitochondrial transit peptide in case in Table I, application no. 12, in column 6 in the same line as SEQ ID NO. 129123 is depicted, mitochondrial is mentioned; which is operable linked, too.

In another embodiment thereof the expression cassette does not comprise an additional transit peptide in case in Table I, application no. 12, in column 6 in the same line as SEQ ID NO. 129123 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 91781, or a homolog or fragment thereof, or a homolog thereof being depicted in Table I, application no. 12, column 8, in the same line as SEQ ID NO. 91781, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 91781 is depicted, plastidic is mentioned, or a mitochondrial transit peptide in case in Table I, application no. 12, in column 6 in the same line as SEQ ID NO. 91781 is depicted, mitochondrial is mentioned; which is operable linked, too.

In another embodiment thereof the expression cassette does not comprise an additional transit peptide in case in Table I, application no. 12, in column 6 in the same line as SEQ ID NO. 91781 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 15175, or a homolog or fragment thereof, or a homolog thereof being depicted in Table I, application no. 12, column 8, in the same line as SEQ ID NO. 15175, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 15175 is depicted, plastidic is mentioned, or a mitochondrial transit peptide in case in Table I, application no. 12, in column 6 in the same line as SEQ ID NO. 15175 is depicted, mitochondrial is mentioned; which is operable linked, too.

In another embodiment thereof the expression cassette does not comprise an additional transit peptide in case in Table I, application no. 12, in column 6 in the same line as SEQ ID NO. 15175 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 129141, or a homolog or fragment thereof, or a homolog thereof being depicted in Table I, application no. 12, column 8, in the same line as SEQ ID NO. 129141, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 129141 is depicted, plastidic is mentioned, or a mitochondrial transit peptide in case in Table I, application no. 12, in column 6 in the same line as SEQ ID NO. 129141 is depicted, mitochondrial is mentioned; which is operable linked, too.

In another embodiment thereof the expression cassette does not comprise an additional transit peptide in case in Table I, application no. 12, in column 6 in the same line as SEQ ID NO. 129141 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 129470, or a homolog or fragment thereof, or a homolog thereof being depicted in Table I, application no. 12, column 8, in the same line as SEQ ID NO. 129470, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 129470 is depicted, plastidic is mentioned, or a mitochondrial transit peptide in case in Table I, application no. 12, in column 6 in the same line as SEQ ID NO. 129470 is depicted, mitochondrial is mentioned; which is operable linked, too.

In another embodiment thereof the expression cassette does not comprise an additional transit peptide in case in Table I, application no. 12, in column 6 in the same line as SEQ ID NO. 129470 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 129546, or a homolog or fragment thereof, or a homolog thereof being depicted in Table I, application no. 12, column 8, in the same line as SEQ ID NO. 129546, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 129546 is depicted, plastidic is mentioned, or a mitochondrial transit peptide in case in Table I, application no. 12, in column 6 in the same line as SEQ ID NO. 129546 is depicted, mitochondrial is mentioned; which is operable linked, too.

In another embodiment thereof the expression cassette does not comprise an additional transit peptide in case in Table I, application no. 12, in column 6 in the same line as SEQ ID NO. 129546 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 129574, or a homolog or fragment thereof, or a homolog thereof being depicted in Table I, application no. 12, column 8, in the same line as SEQ ID NO. 129574, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 129574 is depicted, plastidic is mentioned, or a mitochondrial transit peptide in case in Table I, application no. 12, in column 6 in the same line as SEQ ID NO. 129574 is depicted, mitochondrial is mentioned; which is operable linked, too.

In another embodiment thereof the expression cassette does not comprise an additional transit peptide in case in Table I, application no. 12, in column 6 in the same line as SEQ ID NO. 129574 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 118212, or a homolog or fragment thereof, or a homolog thereof being depicted in Table I, application no. 12, column 8, in the same line as SEQ ID NO. 118212, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 118212 is depicted, plastidic is mentioned, or a mitochondrial transit peptide in case in Table I, application no. 12, in column 6 in the same line as SEQ ID NO. 118212 is depicted, mitochondrial is mentioned; which is operable linked, too.

In another embodiment thereof the expression cassette does not comprise an additional transit peptide in case in Table I, application no. 12, in column 6 in the same line as SEQ ID NO. 118212 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 130181, or a homolog or fragment thereof, or a homolog thereof being depicted in Table I, application no. 12, column 8, in the same line as SEQ ID NO. 130181, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 130181 is depicted, plastidic is mentioned, or a mitochondrial transit peptide in case in Table I, application no. 12, in column 6 in the same line as SEQ ID NO. 130181 is depicted, mitochondrial is mentioned; which is operable linked, too.

In another embodiment thereof the expression cassette does not comprise an additional transit peptide in case in Table I, application no. 12, in column 6 in the same line as SEQ ID NO. 130181 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 128161, or a homolog or fragment thereof, or a homolog thereof being depicted in Table I, application no. 12, column 8, in the same line as SEQ ID NO. 128161, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 128161 is depicted, plastidic is mentioned, or a mitochondrial transit peptide in case in Table I, application no. 12, in column 6 in the same line as SEQ ID NO. 128161 is depicted, mitochondrial is mentioned; which is operable linked, too.

In another embodiment thereof the expression cassette does not comprise an additional transit peptide in case in Table I, application no. 12, in column 6 in the same line as SEQ ID NO. 128161 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 125348, or a homolog or fragment thereof, or a homolog thereof being depicted in Table I, application no. 12, column 8, in the same line as SEQ ID NO. 125348, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 12, in column 6 in the same line as SEQ ID NO. 125348 is depicted, plastidic is mentioned, or a mitochondrial transit peptide in case in Table I, application no. 12, in column 6 in the same line as SEQ ID NO. 125348 is depicted, mitochondrial is mentioned; which is operable linked, too.

In another embodiment thereof the expression cassette does not comprise an additional transit peptide in case in Table I, application no. 12, in column 6 in the same line as SEQ ID NO. 125348 is depicted, non-targeted is mentioned.

[0165.1.8.12] to [0170.1.8.12] for the disclosure of these paragraphs see [0165.1.8.8] to [0170.1.8.8] above.

The invention further provides a vector, comprising a nucleic acid molecule comprising 1) a nucleic acid molecule selected from the group consisting of:
(a) a nucleic acid molecule encoding a polypeptide shown in Table II, column 5 or 8, application no. 12, preferably shown in Table II A, application no. 12, in column 5, or in Table II A, application no. 12, column 8, or in Table II B, application no. 12, column 8, or a homolog or a fragment thereof;
(b) a nucleic acid molecule shown in Table I, application no. 12, column 5 or 8, preferably shown in Table I A, application no. 12, in column 5, or in Table I A, application no. 12, column 8, or in Table I B, application no. 12, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 12, preferably shown in Table II A, application no. 12, in column 5, or in Table II A, application no. 12, column 8, or in Table II B, application no. 12, column 8;
(d) a nucleic acid molecule having at least 30% identity, preferably at least 40%, 50%, 60%, 70%, 75%, 80%, 85%, 90%, 95%, 96%, 97%, 98%, 99%, 99,5%, with the nucleic acid molecule sequence of a polynucleotide comprising the nucleic acid molecule shown in column 5 or 8 of Table I, application no. 12, preferably shown in Table I A, application no. 12, in column 5, or in Table I A, application no. 12, column 8, or in Table I B, application no. 12, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 12;
(i) a nucleic acid molecule encoding a polypeptide having the activity represented by a protein as depicted in column 5 of Table II, application no. 12;
(j) a nucleic acid molecule comprising a polynucleotide which is obtained by amplifying a cDNA library or a genomic library using the primers in Table III, application no. 12, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 12.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 12, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 12.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 12, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 12, in the same line as of the respective nucleic acid molecule according to 1) “plastidic” is depicted, or encoding a mitochondrial transit peptide, especially in case in column 6 of Table I, application no. 12, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 12, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 12, in the same line as of the respective nucleic acid molecule according to 1) “plastidic” is depicted, or encoding a mitochondrial transit peptide, especially in case in column 6 of Table I, application no. 12, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operable linked; a nucleic acid molecule encoding a transit peptide, especially in case in column 6 of Table I, application no. 12, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 12, in the same line as of the respective nucleic acid molecule according to 1) “plastidic” is depicted, or encoding a mitochondrial transit peptide, especially in case in column 6 of Table I, application no. 12, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 12 (in the same line).

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 12, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 12.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 12, in the same line as of the respective nucleic acid molecule according to 1) “plastidic” is depicted, or encoding a mitochondrial transit peptide, especially in case in column 6 of Table I, application no. 12, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 12, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 12, in the same line as of the respective nucleic acid molecule according to 1) “plastidic” is depicted, or encoding a mitochondrial transit peptide, especially in case in column 6 of Table I, application no. 12, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 12 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 12, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 12 (in the same line) and confers a generation or an increase of the respective fine chemical in a non-human organism or a part thereof as compared to a corresponding wild-type non-human organism or part thereof.

In a preferred embodiment of said vector said nucleic acid molecule according to 1 (a), (b), (c), (d), (e), (f), (g), (h), (i), (j) or (k), said promoter and optionally said nucleic acid molecule encoding a transit peptide are encompassed in an expression cassette. Accordingly in an embodiment the invention provides a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

The invention further provides a vector comprising a nucleic acid molecule comprising 1) a nucleic acid molecule selected from the group consisting of:
(a) a nucleic acid molecule encoding a polypeptide shown in Table II, column 5 or 8, application no. 12, preferably shown in Table II A, application no. 12, in column 5, or in Table II A, application no. 12, column 8, or in Table II B, application no. 12, column 8, or a homolog or a fragment thereof;
(b) a nucleic acid molecule shown in Table I, application no. 12, column 5 or 8, preferably shown in Table I A, application no. 12, in column 5, or in Table I A, application no. 12, column 8, or in Table I B, application no. 12, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 12, preferably shown in Table II A, application no. 12, in column 5, or in Table II A, application no. 12, column 8, or in Table II B, application no. 12, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 12, preferably shown in Table I A, application no. 12, in column 5, or in Table I A, application no. 12, column 8, or in Table I B, application no. 12, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 12;
(i) a nucleic acid molecule encoding a polypeptide having the activity represented by a protein as depicted in column 5 of Table II, application no. 12;
(j) a nucleic acid molecule comprising a polynucleotide which is obtained by amplifying a cDNA library or a genomic library using the primers in Table III, application no. 12, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto;
   whereby, preferably, the nucleic acid molecule according to 1 (a), (b), (c), (d), (e), (f), (g), (h), (i), (j) or (k) distinguishes over the sequence depicted in Table I A and/or I B, application no. 12, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 12, column 5 or 8, or the coding region thereof, respectively. In another embodiment, the nucleic acid molecule of the present invention, preferably the coding region thereof, is 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 12, column 5 or 8, or the coding region thereof, respectively. In a further embodiment the nucleic acid molecule does not encode the polypeptide sequence shown in Table II A and/or II B, application no. 12, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 12, column 5 or 8 and does not encode a protein of the sequence shown in Table II A and/or II B, application no. 12, column 5 or 8.

Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 12, column 5 and 8, or the coding region thereof. In a further embodiment, the protein of the present invention is at least 30%,40%, 50%, 60%, 70%, 80%, 90%, 95% identical to protein sequence depicted in Table II A and/or II B, application no. 12, column 5 or 8 but less than 100%, preferably less than 99.999%, 99.99% or 99.9%, more preferably less than 99%, 985, 97%, 96% or 95% identical to the sequence shown in Table II A and/or II B, application no. 12, column 5 or 8.

(All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 12.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 12, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 12.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 12, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 12, in the same line as of the respective nucleic acid molecule according to 1) “plastidic” is depicted, or encoding a mitochondrial transit peptide, especially in case in column 6 of Table I, application no. 12, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 12, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 12, in the same line as of the respective nucleic acid molecule according to 1) “plastidic” is depicted, or encoding a mitochondrial transit peptide, especially in case in column 6 of Table I, application no. 12, in the same line as of the respective nucleic acid molecule according to 1) “mitochondrial” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 12, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 12, in the same line as of the respective nucleic acid molecule according to 1) “plastidic” is depicted, or encoding a mitochondrial transit peptide, especially in case in column 6 of Table I, application no. 12, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 12.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 12, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 12.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 12, in the same line as of the respective nucleic acid molecule according to 1) “plastidic” is depicted, or encoding a mitochondrial transit peptide, especially in case in column 6 of Table I, application no. 12, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 12, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 12, in the same line as of the respective nucleic acid molecule according to 1) “plastidic” is depicted, or encoding a mitochondrial transit peptide, especially in case in column 6 of Table I, application no. 12, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 12 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 12, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 12 and confers a generation or an increase of the respective fine chemical in a non-human organism or a part thereof as compared to a corresponding wild-type non-human organism or part thereof.

[0181.1.8.12] to [0209.1.8.12] for the disclosure of these paragraphs see [0181.1.8.8] to [0209.1.8.8] above.

The genes of the invention, coding for an activity selected from the group consisting of 2,3-dihydroxyphenylpropionate 1,2-dioxygenase, 2-C-methyl-D-erythritol 4-phosphate cytidylyltransferase, 30S ribosomal protein S3, 3-ketoacyl-CoA thiolase, 3-oxoacyl-[acyl-carrier-protein] reductase, 3-oxoacyl-[acyl-carrier-protein] synthase, 40S ribosomal protein S17, ABC transporter component, ABC transporter permease protein, acetyl-coenzyme A synthetase, acid shock protein, acyl-CoA synthase, acyl-desaturase, adenosine kinase, aldolase, alkaline phosphatase isozyme conversion protein, amino acid ABC transporter permease protein, aminotransferase, ankyrin repeat protein, At2g33510-protein, ATPase epsilon subunit, ATP-binding component of a transport system, b0050-protein, b0249-protein, b0371-protein, b0518-protein, b0801-protein, b1046-protein, b1346-protein, b1470-protein, b1522-protein, b1670-protein, b1703-protein, b1837-protein, b2032-protein, b2099-protein, b2107-protein, b2126-protein, b2139-protein, b2360-protein, b2474-protein, b2513-protein, b2654-protein, b3442-protein, b3644-protein, b3812-protein, b3944-protein, b4023-protein, b4205-protein, beta-hydroxylase, betaine aldehyde dehydrogenase, bifunctional protein (phosphoribosyltransferase and regulatory protein), bifunctional purine biosynthesis protein, binding-protein-dependent transport systerns inner membrane component, biotin synthase, calcium-dependent protein kinase, carbon dioxide concentrating mechanism protein, cardiolipin synthase, CDP-diacylglycerol-serine O-phosphatidyltransferase, cell division control protein, cell wall protein, chlorophyllase, citrate synthase, coproporphyrinogen III oxidase, cyclin, cystathionine gamma-synthase, cysteine syn-thase A, dihydrolipoamide dehydrogenase, DNA mismatch repair protein, DnaJ-like chaperone, electron transport complex protein, Elongation factor Tu, ethanolamine utilization protein, F-box protein, ferredoxin, fructose 1,6-bisphosphatase, fumarylacetoacetate hydrolase, glutamate 5-kinase, glutamate-ammonia-ligase, glutaredoxin, glycerol-3-phosphate dehydrogenase, glycine cleavage system H protein, GM02LC38418-protein, heat shock protein, heat shock transcription factor, HesB/YadR/YfhF family protein, homocitrate synthase, hydrolase, isocitrate dehydrogenase, lipoprotein precursor, malate dehydrogenase, malonyl CoA-acyl carrier protein transacylase, metal-dependent hydrolase, molecular chaperone, molecular chaperone portein, monothiol glutaredoxin, multiple antibiotic resistance protein, murein transglycosylase, N-acetyl-gamma-glutamyl-phosphate reductase, NADP-dependent malic enzyme, outer-membrane lipoprotein precursor, oxidoreductase, permease protein of ABC transporter, phage-related repressor protein, phosphatase, phosphate import ATP-binding protein, phosphatidylserine synthase, phospho-2-dehydro-3-deoxyheptonate aldolase, phosphoadenosine phosphosulfate reductase, phosphotransferase, phosphotransferase system component, polygalacturonase, protein kinase, protein phosphatase, purine nucleoside phosphorylase, pyridoxamine kinase, recombinase A, sec-independent protein translocase, Sec-independent protein translocase subunit, sensor histidine kinase, sensor kinase, sll0281-protein, sll0406-protein, small nuclear ribonucleoprotein, sodium/proton antiporter, squalene monooxygenase, sugar transport system permease protein, threonine dehydratase, transcription factor, transcriptional regulator, transcriptional regulator protein, transport protein, tryptophan synthase alpha chain, TTC1386-protein, vacuolar processing enzyme, ycl027w-protein, ycl049c-protein, ydr273w-protein, ydr339c-protein, YFL019C-protein, ygl237c-protein, YGR104C-protein, yhr033w-protein, YJL127W-A-protein, ylr326w-protein, yml083c-protein, yml084w-protein, and ynl321w-protein are also called “FCRP genes”.

[0211.1.8.12] to [0225.1.8.12] for the disclosure of these paragraphs see [0211.1.8.8] to [0225.1.8.8] above.

In addition to the sequence mentioned in Table I, application no. 12, column 5 or 8, preferably the coding region thereof, or its homologs or fragments, it may be advantageous additionally to express and/or mutate further genes in the non-human organisms. Especially advantageously, additionally at least one further gene of the biosynthetic pathway of the fine chemical is expressed in the non-human organisms such as plants or microorganisms. It is also possible that the regulation of the natural genes has been modified advantageously so that the gene and/or its gene product is no longer subject to the regulatory mechanisms which exist in the non-human organisms. This leads to an increased synthesis of the respective fatty acid since, for example, feedback regulations no longer exist to the same extent or not at all. In addition it might be advantageously to combine the nucleic acids sequences of the invention containing the sequences shown in the respective line in Table I, application no. 12, column 5 or 8, preferably the coding region thereof, or homologs or fragments thereof, with genes which generally support or enhance the growth or yield of the target non-human organisms, for example genes which lead to faster growth rate of non human organism like microorganisms or plants or genes which produce stress-, pathogen-, or herbicide-resistant plants.

[0227.1.8.12] to [0239.1.8.12] for the disclosure of these paragraphs see [0227.1.8.8] to [0239.1.8.8] above.

In an embodiment of the present invention the nucleic acid construct of the present invention comprises a nucleic acid molecule as depicted in the respective line in Table I, application no. 12, column 5 or 8, preferably the coding region thereof, a fragment or a homolog thereof, and a plant promoter, such as the Big 35S promoter, the DC3 promoter, the PC Ubi promoter, the LegB4-promoter, the Super promoter, the USP promoter or the like. In another embodiment the nucleic acid construct of the present invention comprises a nucleic acid molecule as depicted in the respective line in Table I, application no. 12, column 5 or 8, preferably the coding region thereof, a fragment or a homolog thereof, and a plant promoter such as, for example, the USP, the LegB4-, the DC3 promoter or the ubiquitin promoter from parsley or other herein mentioned promoters. In a further embodiment the nucleic acid construct of the present invention comprises a nucleic acid molecule as depicted in the respective line in Table I, application no. 12, column 5 or 8, preferably the coding region thereof, a fragment or a homolog thereof, and a plant promoter, such as the Big 35S promoter, the PC Ubi promoter, the Super promoter, the USP promoter or the like. In addition different terminators may advantageously be used in these nucleic acid constructs; examples for transcriptional termination are polyadenylation signals.

[0241.1.8.12] to [0245.1.8.12] for the disclosure of these paragraphs see [0241.1.8.8] to [0245.1.8.8] above.

Other preferred sequences for use in operable linkage in gene expression constructs are targeting sequences, which are required for targeting the gene product into specific cell compartments (for a review, see Kermode, Crit. Rev. Plant Sci. 15 (4), 285 (1996) and references cited therein), for example into the vacuole, the nucleus, all types of plastids, such as amyloplasts, chloroplasts, chromoplasts, the extracellular space, the mitochondria, the endoplasmic reticulum, elaioplasts, peroxisomes, glycosomes, and other compartments of cells or extracellular; preferred are sequences, which are involved in targeting to plastids as mentioned above. Sequences, which must be mentioned in this context are, in particular, the signal-peptide- or transit-peptide-encoding sequences which are known per se. For example, plastid-transit-peptide-encoding sequences enable the targeting of the expression product into the plastids of a plant cell. Targeting sequences are also known for eukaryotic and to a lower extent for prokaryotic organisms and can advantageously be operable linked with the nucleic acid molecule of the present invention as shown in Table I, application no. 12, columns 5 and 8, preferably the coding region thereof, or homologs or fragments thereof, and described herein to achieve an expression in one of said compartments or extracellular.

[0247.1.8.12] to [0266.1.8.12] for the disclosure of these paragraphs see [0247.1.8.8] to see [0266.1.8.8] above

An embodiment of the invention relates to a vector where the nucleic acid molecule according to the invention is linked operably to regulatory sequences which permit the expression in a prokaryotic or eukaryotic host. A further embodiment of the invention relates to a vector in which a nucleic acid sequence encoding one of the polypeptides shown in the respective line in Table II, application no. 12, columns 5 or 8, or homologs or fragments thereof, is functionally linked to a targeting sequence such as a plastidial or mitochondrial targeting sequence. A further embodiment of the invention relates to a vector in which a nucleic acid sequence encoding one of the polypeptides shown in the respective line in Table II, application no. 12, columns 5 or 8, or homologs or fragments thereof, is functionally linked to (a) regulatory sequence which permit the expression in an organelle, such as plastids or mitochondria, preferably plastids.

A further embodiment of the invention relates to a vector which comprises a nucleic acid sequence encoding one of the polypeptides shown in the respective line in Table II, application no. 12, columns 5 or 8, or homologs or fragments thereof, but no additional targeting sequence.

[0268.1.8.12] to [0273.1.8.12] for the disclosure of these paragraphs see [0268.1.8.8] to [0273.1.8.8] above.

A further object of the invention relates to the use of a nucleic acid construct, e.g. an expression cassette, comprising one or more nucleic acid molecules encoding one or more polypeptides as depicted in the respective line(s) in Table II, application no. 12, columns 5 or 8, or homologs or fragments thereof, or comprising one or more nucleic acid molecules as depicted in the respective line(s) in Table I, application no. 12, columns 5 or 8, preferably the coding region thereof, or homologs or fragments thereof, or comprising a nucleic acid molecule hybridizing therewith for the transformation of a microorganism or a plant cell, plant tissues or a part of a plant.

In doing so, depending on the choice of promoter, the nucleic acid molecules shown in the respective line in Table I, application no. 12, column 5 or 8, preferably the coding region thereof, or homologs or fragments thereof, or the nucleic acid molecules encoding a polypeptide as depicted in the respective line in Table II, application no. 12, column 5 or 8, or homologs or fragments thereof, can be expressed in the whole plant or specifically e.g. in the leaves, in the seeds, the nodules, in roots, in the stem or other parts of the plant. Those transgenic plants overproducing sequences, e.g. as depicted in the respective line in Table I, application no. 12, column 5 or 8, preferably the coding region thereof, or homologs or fragments thereof, the plant cell, plant tissues, plants, the reproductive material thereof, or parts thereof are a further object of the present invention.

Within the framework of the present invention, the production of the 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid is due to the generation or over-expression of one or more polypeptides as depicted in the respective line(s) in Table II, application no. 12, column 5 or 8, or homologs or fragments thereof, or encoded by the corresponding nucleic acid molecules as depicted in the respective line(s) in Table I, application no. 12, column 5 or 8, preferably the coding region thereof, or homologs or fragments thereof, in the non-human organism according to the invention, advantageously in the transgenic microorganism or plant according to the invention, at least for the duration of at least one plant generation.

A constitutive expression of the polypeptide as depicted in the respective line in Table II, application no. 12, column 5 or 8, or homologs or fragments thereof, or encoded by the respective nucleic acid molecule as depicted in the respective line in Table I, application no. 12, column 5 or 8, preferably the coding region thereof, or homologs or fragments thereof, may be advantageous. On the other hand, an inducible expression may also appear desirable.

Expression of the polypeptide sequences of the invention can be either directed to the cytosol or to the organelles, such as plastids or mitochondria, preferably the plastids of the host cells, preferably the plant cells, or non-targeted.

for the disclosure of this paragraph see [0278.1.8.8] above.

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 12, column 5 or 8, preferably shown in Table II A, application no. 12, column 5, or in Table II A, application no. 12, column 8, or in Table II B, application no. 12, column 8, or a homolog or a fragment thereof;
   (b) a nucleic acid molecule shown in Table I, application no. 12, column 5 or 8, preferably shown in Table I A, application no. 12, column 5, or in Table I A, application no. 12, column 8, or in Table I B, application no. 12, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 12, preferably shown in Table II A, application no. 12, column 5, or in Table II A, application no. 12, column 8, or in Table II B, application no. 12, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 12, preferably shown in Table I A, application no. 12, column 5, or in Table I A, application no. 12, column 8, or in Table I B, application no. 12, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 12;
   (i) a nucleic acid molecule encoding a polypeptide having the activity represented by a respective protein as depicted in column 5 of Table II, application no. 12;
   (j) a nucleic acid molecule comprising a polynucleotide which is obtained by amplifying a cDNA library or a genomic library using the primers in Table III, application no. 12, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto; and, if desired
2) nucleic acid molecule encoding a transit peptide, preferably encoding a plastidal transit peptide or a mitochondrial transit peptide;
   which are operable linked;]
   or comprising an expression cassette comprising
[0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 12, column 5 or 8, preferably shown in Table II A, application no. 12, column 5, or in Table II A, application no. 12, column 8, or in Table II B, application no. 12, column 8, or a homolog or a fragment thereof;
   (b) a nucleic acid molecule shown in Table I, application no. 12, column 5 or 8, preferably shown in Table I A, application no. 12, column 5, or in Table I A, application no. 12, column 8, or in Table I B, application no. 12, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 12, preferably shown in Table II A, application no. 12, column 5, or in Table II A, application no. 12, column 8, or in Table II B, application no. 12, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 12, preferably shown in Table I A, application no. 12, column 5, or in Table I A, application no. 12, column 8, or in Table I B, application no. 12, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 12;
   (i) a nucleic acid molecule encoding a polypeptide having the activity represented by a respective protein as depicted in column 5 of Table II, application no. 12;
   (j) a nucleic acid molecule comprising a polynucleotide which is obtained by amplifying a cDNA library or a genomic library using the primers in Table III, application no. 12, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide; which are operable linked;]
   or comprising or being transformed by a vector comprising
[1) a nucleic acid molecule selected from the group consisting of
   (a) a nucleic acid molecule encoding a polypeptide shown in Table II, application no. 12, column 5 or 8, preferably shown in Table II A, application no. 12, column 5, or in Table II A, application no. 12, column 8, or in Table II B, application no. 12, column 8, or a homolog or a fragment thereof;
   (b) a nucleic acid molecule shown in Table I, application no. 12, column 5 or 8, preferably shown in Table I A, application no. 12, column 5, or in Table I A, application no. 12, column 8, or in Table I B, application no. 12, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 12, preferably shown in Table II A, application no. 12, column 5, or in Table II A, application no. 12, column 8, or in Table II B, application no. 12, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 12, preferably shown in Table I A, application no. 12, column 5, or in Table I A, application no. 12, column 8, or in Table I B, application no. 12, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 12;
   (i) a nucleic acid molecule encoding a polypeptide having the activity represented by a respective protein as depicted in column 5 of Table II, application no. 12;
   (j) a nucleic acid molecule comprising a polynucleotide which is obtained by amplifying a cDNA library or a genomic library using the primers in Table III, application no. 12, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 12.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 12 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 12, in the same line as of the respective nucleic acid molecule according to 1) “plastidic” is depicted, or encoding a mitochondrial transit peptide, especially in case in column 6 of Table I, application no. 12, in the same line as of the respective nucleic acid molecule according to 1) “mitochondrial” is depicted.

In another embodiment of said transgenic non-human organism the expression cassette does not comprise 2) a nucleic acid molecule encoding a transit peptide, especially in case in column 6 of Table I, application no. 12, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 12.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 12, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 12, in the same line as of the respective nucleic acid molecule according to 1) “plastidic” is depicted, or encoding a mitochondrial transit peptide, especially in case in column 6 of Table I, application no. 12, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 12, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 12, in the same line as of the respective nucleic acid molecule according to 1) “plastidic” is depicted, or encoding a mitochondrial transit peptide, especially in case in column 6 of Table I, application no. 12, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 12, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 12, in the same line as of the respective nucleic acid molecule according to 1) “plastidic” is depicted, or encoding a mitochondrial transit peptide, especially in case in column 6 of Table I, application no. 12, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 12.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 12, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 12.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 12, in the same line as of the respective nucleic acid molecule according to 1) “plastidic” is depicted, or encoding a mitochondrial transit peptide, especially in case in column 6 of Table I, application no. 12, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 12, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 12, in the same line as of the respective nucleic acid molecule according to 1) “plastidic” is depicted, or encoding a mitochondrial transit peptide, especially in case in column 6 of Table I, application no. 12, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 12 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 12, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 12 (in the same line) and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 12, column 5 or 8, preferably shown in Table II A, application no. 12, column 5, or in Table II A, application no. 12, column 8, or in Table II B, application no. 12, column 8, or a homolog or a fragment thereof;
   (b) a nucleic acid molecule shown in Table I, application no. 12, column 5 or 8, preferably shown in Table I A, application no. 12, column 5, or in Table I A, application no. 12, column 8, or in Table I B, application no. 12, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 12, preferably shown in Table II A, application no. 12, column 5, or in Table II A, application no. 12, column 8, or in Table II B, application no. 12, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 12, preferably shown in Table I A, application no. 12, column 5, or in Table I A, application no. 12, column 8, or in Table I B, application no. 12, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 12;
   (i) a nucleic acid molecule encoding a polypeptide having the activity represented by a respective protein as depicted in column 5 of Table II, application no. 12;
   (j) a nucleic acid molecule comprising a polynucleotide which is obtained by amplifying a cDNA library or a genomic library using the primers in Table III, application no. 12, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising an expression cassette comprising
[0) a promoter, preferably a promoter selected from the group consisting of Big35S, PCUbi, Super and USP; and 1) a nucleic acid molecule selected from the group consisting of
   (a) a nucleic acid molecule encoding a polypeptide shown in Table II, application no. 12, column 5 or 8, preferably shown in Table II A, application no. 12, column 5, or in Table II A, application no. 12, column 8, or in Table II B, application no. 12, column 8, or a homolog or a fragment thereof;
   (b) a nucleic acid molecule shown in Table I, application no. 12, column 5 or 8, preferably shown in Table I A, application no. 12, column 5, or in Table I A, application no. 12, column 8, or in Table I B, application no. 12, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 12, preferably shown in Table II A, application no. 12, column 5, or in Table II A, application no. 12, column 8, or in Table II B, application no. 12, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 12, preferably shown in Table I A, application no. 12, column 5, or in Table I A, application no. 12, column 8, or in Table I B, application no. 12, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 12;
   (i) a nucleic acid molecule encoding a polypeptide having the activity represented by a respective protein as depicted in column 5 of Table II, application no. 12;
   (j) a nucleic acid molecule comprising a polynucleotide which is obtained by amplifying a cDNA library or a genomic library using the primers in Table III, application no. 12, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      and, if desired
2) nucleic acid molecule encoding a transit peptide, preferably encoding a plastidal transit peptide or a mitochondrial transit peptide; which are operable linked;]
   or comprising or being transformed by a vector comprising
[1) a nucleic acid molecule selected from the group consisting of
   (a) a nucleic acid molecule encoding a polypeptide shown in Table II, application no. 12, column 5 or 8, preferably shown in Table II A, application no. 12, column 5, or in Table II A, application no. 12, column 8, or in Table II B, application no. 12, column 8, or a homolog or a fragment thereof;
   (b) a nucleic acid molecule shown in Table I, application no. 12, column 5 or 8, preferably shown in Table I A, application no. 12, column 5, or in Table I A, application no. 12, column 8, or in Table I B, application no. 12, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 12, preferably shown in Table II A, application no. 12, column 5, or in Table II A, application no. 12, column 8, or in Table II B, application no. 12, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 12, preferably shown in Table I A, application no. 12, column 5, or in Table I A, application no. 12, column 8, or in Table I B, application no. 12, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 12;
   (i) a nucleic acid molecule encoding a polypeptide having the activity represented by a respective protein as depicted in column 5 of Table II, application no. 12;
   (j) a nucleic acid molecule comprising a polynucleotide which is obtained by amplifying a cDNA library or a genomic library using the primers in Table III, application no. 12, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      whereby, preferably, the nucleic acid molecule according to 1 (a), (b), (c), (d), (e), (f), (g), (h), (i), (j) or (k) distinguishes over the sequence depicted in Table I A and/or I B, application no. 12, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 12, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 12, column 5 or 8, or the coding region thereof, respectively. In a further embodiment the nucleic acid molecule does not encode the polypeptide sequence shown in Table II A and/or II B, application no. 12, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 12, column 5 or 8 and does not encode a protein of the sequence shown in Table II A and/or II B, application no. 12, column 5 or 8. Accordingly, in one embodiment, the protein encoded by a sequence of a nucleic acid according to 1 (a), (b), (c), (d), (e), (f), (g), (h), (i), (j) or (k) does not consist of the sequence shown in Table I A and/or I B, application no. 12, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 12, column 5 or 8 but less than 100%, preferably less than 99.999%, 99.99%, 99.9%, 99%, 98%, 97%, 96% or 95% identical to the sequence shown in Table II A and/or II B, application no. 12, column 5 or 8;
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 12.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 12 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 12, in the same line as of the respective nucleic acid molecule according to 1) “plastidic” is depicted, or encoding a mitochondrial transit peptide, especially in case in column 6 of Table I, application no. 12, in the same line as of the respective nucleic acid molecule according to 1) “mitochondrial” is depicted.

In another embodiment thereof the expression cassette does not comprise 2) a nucleic acid molecule encoding a transit peptide, especially in case in column 6 of Table I, application no. 12, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 12.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the same line in column 5 of Table II, application no. 12, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 12, in the same line as of the respective nucleic acid molecule according to 1) “plastidic” is depicted, or encoding a mitochondrial transit peptide, especially in case in column 6 of Table I, application no. 12, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 12, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 12, in the same line as of the respective nucleic acid molecule according to 1) “plastidic” is depicted, or encoding a mitochondrial transit peptide, especially in case in column 6 of Table I, application no. 12, in the same line as of the respective nucleic acid molecule according to 1) “mitochondrial” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 12, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 12, in the same line as of the respective nucleic acid molecule according to 1) “plastidic” is depicted, or encoding a mitochondrial transit peptide, especially in case in column 6 of Table I, application no. 12, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 12.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 12, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 12.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 12, in the same line as of the respective nucleic acid molecule according to 1) “plastidic” is depicted, or encoding a mitochondrial transit peptide, especially in case in column 6 of Table I, application no. 12, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 12, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 12, in the same line as of the respective nucleic acid molecule according to 1) “plastidic” is depicted, or encoding a mitochondrial transit peptide, especially in case in column 6 of Table I, application no. 12, in the same line as of the respective nucleic acid molecule according to 1) “mitochondrial” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 12 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 12, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 12 and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

[0291.1.8.12] to [0299.1.8.12] for the disclosure of these paragraphs see [0291.1.8.8] to [0299.1.8.8] above.

The term “biological active portion” or “biological activity” means a polypeptide as depicted in the respective line in Table II, application no. 12, column 3 or a portion of said polypeptide which still has at least 10% or 20%, preferably 30%, 40%, 50% or 60%, especially preferably 70%, 75%, 80%, 90% or 95% of the enzymatic or biological activity of the natural or starting enzyme or protein.

[0301.1.8.12] to [0304.1.8.12] for the disclosure of these paragraphs see [0301.1.8.8] to [0304.1.8.8] above.

Synthetic oligonucleotide primers for the amplification, e.g. as shown in the respective line in Table III, application no. 12, column 8, by means of polymerase chain reaction can be generated on the basis of a sequence shown herein, for example the sequence shown in the respective line in Table I, application no. 12, columns 5 or 8, or the sequences derived from Table II, application no. 12, columns 5 or 8, respectively.

Moreover, it is possible to identify a conserved region of a protein from various organism by carrying out protein sequence alignments with the polypeptide encoded by the nucleic acid molecules of the present invention, in particular with the sequences encoded by the nucleic acid molecule shown in the respective line in column 5 or 8 of Table I, application no. 12, from which conserved regions, and in turn, degenerate primers can be derived. Conserved regions are those, which show a very little variation in the amino acid in one particular position of several homologs from different origin. The consensus sequence and polypeptide motifs shown in the respective line in column 8 of Table IV, application no. 12, are derived from such alignments of homologous proteins.

In an embodiment of the present invention, in the method of the present invention the activity of a polypeptide comprising or consisting of a consensus sequence or at least one polypeptide motif shown in the respective line in Table IV, application no. 12, column 8, is increased. In another embodiment thereof, the method of the present invention relates to a polypeptide comprising or consisting of a consensus sequence shown in the respective line in Table IV, application no. 12, column 8 or comprising at least one polypeptide motif shown in the respective line in Table IV, application no. 12, column 8, whereby not more than 20, 15, 10, 9, 8, 7, 6, 5 4, 3, 2 or 1, or 0 of the amino acid positions indicated can be replaced by any amino acid. In another embodiment thereof, the method of the present invention relates to a polypeptide comprising or consisting of a consensus sequence shown in the respective line in Table IV, application no. 12, column 8 or comprising at least one polypeptide motif shown in the respective line in Table IV, application no. 12, column 8, whereby not more than 15%, 10%, 5%, 4%, 3%, 2% or 1%, or 0% of the amino acid positions defined as distinct specific amino acids are/is replaced by another amino acid. In another embodiment thereof, the method of the present invention relates to a polypeptide comprising or consisting of a consensus sequence shown in the respective line in Table IV, application no. 12, column 8 or comprising at least one polypeptide motif shown in the respective line in Table IV, application no. 12, column 8, whereby not more than 20, 15, 10, 9, 8, 7, 6, 5, 4, 3, 2 or 1, or 0 amino acids are inserted into a consensus sequence or polypeptide motif.

In an embodiment of the present invention, in the method of the present invention the activity of a polypeptide comprising or consisting of a consensus sequence or at least one polypeptide motif shown in the respective line in Table IV, application no. 12, column 8, is increased, whereby said polypeptide distinguishes over the sequence depicted in Table II, application no. 12, columns 5 or 8 by one or more amino acids.

In one embodiment, said polypeptide of the invention distinguishes over the sequence shown in the respective line in Table II, application no. 12, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II B, application no. 12, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in Table II, application no. 12, columns 5 or 8.

[0309.1.8.12] to [0321.1.8.12] for the disclosure of these paragraphs see [0309.1.8.8] to [0321.1.8.8] above.

Polypeptides having above-mentioned activity, i.e. conferring the production or the increased production of the fine chemical 2-hydroxybehenic acid, 2-hydroxypalmitic acid, 2hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid, respectively, as compared to a corresponding, e.g. non-transformed, wild type non-human organism, like a microorganism or a plant cell, plant or part thereof, can be encoded by other DNA sequences which hybridize to the sequences shown in the respective line in Table I, application no. 12, columns 5 and 8, preferably the coding region thereof, at least under relaxed hybridization conditions and which encode the expression of polypeptides conferring the production or the increased production of the respective fine chemical 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid, respectively, as compared to a corresponding, e.g. non-transformed, wild type non-human organism, like a microorganism or a plant cell, plant or part thereof. [0323.1.8.12] to [0329.1.8.12] for the disclosure of these paragraphs see [0323.1.8.8] to [0329.1.8.8] above.

Further, the nucleic acid molecule of the invention comprises a nucleic acid molecule, which is a complement of one of the nucleotide sequences of above-mentioned nucleic acid molecules or a portion thereof. A nucleic acid molecule or its sequence which is complementary to one of the nucleotide molecules or sequences shown in the respective line in Table I, application no. 12, columns 5 or 8, preferably the coding region thereof, homologs or fragments thereof, is one which is sufficiently complementary to one of the nucleotide molecules or sequences shown in the respective line in Table I, application no. 12, columns 5 or 8, preferably the coding region thereof, homologs or fragments thereof, such that it can hybridize to one of the nucleotide sequences shown in Table I, application no. 12, columns 5 or 8, preferably the coding region thereof, homologs or fragments thereof, thereby forming a stable duplex. Preferably, the hybridization is performed under stringent hybridization conditions. However, a complement of one of the herein disclosed sequences is preferably a sequence complement thereto according to the base pairing of nucleic acid molecules well known to the skilled person. For example, the bases A and G undergo base pairing with the bases T and U or C, resp. and visa versa. Modifications of the bases can influence the base-pairing partner.

The nucleic acid molecule of the invention comprises a nucleotide sequence which is at least about 30%, 35%, 40%, 45%, 50%, 55%, 60%, 65%, 70%, 75%, 80%, 90%, 95%, 96%, 97%, 98%, 99%, 99.5% or more homologous to a nucleotide sequence shown in Table I, application no. 12, columns 5 or 8, preferably the coding region thereof, homologs or fragments thereof, and preferably has above-mentioned activity, in particular conferring the pro duction or the increased production of the fine chemical 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid, respectively, after increasing the activity or an activity of a gene as shown in the respective line in Table I or of a gene product, e.g. as shown in the respective line in Table II, application no. 12, column 5 or 8, by for example in one embodiment expression either in the cytosol or in an organelle such as a plastid or mitochondria or both, preferably in a plastid, or in another embodiment non-targeted or targeted.

The nucleic acid molecule of the invention comprises a nucleotide sequence or molecule which hybridizes, preferably hybridizes under stringent conditions as defined herein, to one of the nucleic acid molecule shown in the respective line in Table I, preferably Table IB, application no. 12, columns 5 or 8, preferably the coding region thereof, fragments or homologs thereof, and encodes a protein having above-mentioned activity, e.g. conferring the production or the increased production of the respective fine chemical 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted, and optionally, the activity thereof is selected from the group consisting of 2,3-dihydroxyphenylpropionate 1,2-dioxygenase, 2-C-methyl-D-erythritol 4-phosphate cytidylyltransferase, 30S ribosomal protein S3, 3-ketoacyl-CoA thiolase, 3-oxoacyl-[acyl-carrier-protein] reductase, 3-oxoacyl-[acyl-carrier-protein] synthase, 40S ribosomal protein S17, ABC transporter component, ABC transporter permease protein, acetyl-coenzyme A synthetase, acid shock protein, acyl-CoA synthase, acyl-desaturase, adenosine kinase, aldolase, alkaline phosphatase isozyme conversion protein, amino acid ABC transporter permease protein, aminotransferase, ankyrin repeat protein, At2g33510-protein, ATPase epsilon subunit, ATP-binding component of a transport system, b0050-protein, b0249-protein, b0371-protein, b0518-protein, b0801-protein, b1046-protein, b1346-protein, b1470-protein, b1522-protein, b1670-protein, b1703-protein, b1837-protein, b2032-protein, b2099-protein, b2107-protein, b2126-protein, b2139-protein, b2360-protein, b2474-protein, b2513-protein, b2654-protein, b3442-protein, b3644-protein, b3812-protein, b3944-protein, b4023-protein, b4205-protein, beta-hydroxylase, betaine aldehyde dehydrogenase, bifunctional protein (phosphoribosyltransferase and regulatory protein), bifunctional purine biosynthesis protein, binding-protein-dependent transport systems inner membrane component, biotin synthase, calcium-dependent protein kinase, carbon dioxide concentrating mechanism protein, cardiolipin synthase, CDP-diacylglycerol-serine O-phosphatidyltransferase, cell division control protein, cell wall protein, chlorophyllase, citrate synthase, coproporphyrinogen III oxidase, cyclin, cystathionine gamma-synthase, cysteine synthase A, dihydrolipoamide dehydrogenase, DNA mismatch repair protein, DnaJ-like chaperone, electron transport complex protein, Elongation factor Tu, ethanolamine utilization protein, F-box protein, ferredoxin, fructose 1,6-bisphosphatase, fumarylacetoacetate hydrolase, glutamate 5-kinase, glutamate-ammonia-ligase, glutaredoxin, glycerol-3-phosphate dehydrogenase, glycine cleavage system H protein, GM02LC38418-protein, heat shock protein, heat shock transcription factor, HesB/YadR/YfhF family protein, homocitrate synthase, hydrolase, isocitrate dehydrogenase, lipoprotein precursor, malate dehydrogenase, malonyl CoAacyl carrier protein transacylase, metal-dependent hydrolase, molecular chaperone, molecular chaperone portein, monothiol glutaredoxin, multiple antibiotic resistance protein, murein transglycosylase, N-acetyl-gamma-glutamyl-phosphate reductase, NADP-dependent malic enzyme, outer-membrane lipoprotein precursor, oxidoreductase, permease protein of ABC transporter, phage-related repressor protein, phosphatase, phosphate import ATP-binding protein, phosphatidylserine synthase, phospho-2-dehydro-3-deoxyheptonate aldolase, phosphoadenosine phosphosulfate reductase, phosphotransferase, phosphotransferase system component, polygalacturonase, protein kinase, protein phosphatase, purine nucleoside phosphorylase, pyridoxamine kinase, recombinase A, sec-independent protein translocase, Sec-independent protein translocase subunit, sensor histidine kinase, sensor kinase, sll0281-protein, sll0406-protein, small nuclear ribonucleoprotein , sodium/proton antiporter, squalene monooxygenase, sugar transport system permease protein, threonine dehydratase, transcription factor, transcriptional regulator, transcriptional regulator protein, transport protein, tryptophan synthase alpha chain, TTC1386-protein, vacuolar processing enzyme, ycl027w-protein, ycl049c-protein, ydr273w-protein, ydr339c-protein, YFL019C-protein, ygl237c-protein, YGR104C-protein, yhr033w-protein, YJL127W-A-protein, ylr326w-protein, yml083c-protein, yml084w-protein, and ynl321w-protein, respectively.

Moreover, the nucleic acid molecule of the invention can comprise only a portion of the coding region of one of the nucleic acid molecules depicted in Table I, application no. 12, columns 5 or 8, for example a fragment which can be used as a probe or primer or a fragment encoding a biologically active portion of the polypeptide of the present invention or of a polypeptide used in the process of the present invention, i.e. having above-mentioned activity, e.g. conferring the production or the increased production of the respective fine chemical 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted. The nucleotide sequences determined from the cloning of the present protein-according-to-the-invention-encoding gene allows for the generation of probes and primers designed for use in identifying and/or cloning its homologues from other cell types and organisms. The probe/primer typically comprises a substantially purified oligonucleotide. The oligonucleotide typically comprises a region of nucleotide sequence that hybridizes under stringent conditions to at least about 12, 15 preferably about 20 or 25, more preferably about 40, 50 or 75 consecutive nucleotides of a sense strand of one of the sequences set forth, e.g., in the respective line in Table I, application no. 12, column 5 or 8, preferably the coding region thereof, or a homolog or a fragment thereof, an anti-sense sequence of one of the sequences, e.g., set forth in the respective line in Table I, application no. 12, columns 5 or 8, preferably the coding region thereof, or a homolog or a fragment thereof or naturally occurring mutants thereof. Primers based on a nucleic acid molecule of the invention can be used in PCR reactions to clone homologs of the polypeptide of the invention or of the polypeptide used in the process of the invention, e.g. as the primers described in the examples of the present invention, e.g. as shown in the examples. A PCR with the primers shown in the respective line in Table III, column 8 will result in a fragment of the gene product as shown in Table II, application no. 12, column 5.

Primer sets are interchangeable. The person skilled in the art knows how to design and combine said primers to result in the desired product, e.g. in a full length clone or a partial sequence. Probes based on the sequences of the nucleic acid molecule of the invention or used in the process of the present invention can be used to detect transcripts or genomic sequences encoding the same or homologous proteins. The probe can further comprise a label group attached thereto, e.g. the label group can be a radioisotope, a fluorescent compound, an enzyme, or an enzyme co-factor. Such probes can be used as a part of a genomic marker test kit for identifying cells which express a polypeptide of the invention or used in the process of the present invention, such as by measuring a level of an encoding nucleic acid molecule in a sample of cells, e.g. detecting mRNA levels or determining, whether a genomic gene comprising the sequence of the polynucleotide of the invention or used in the processes of the present invention has been mutated or deleted.

As the expression of a respective nucleic acids of the invention is related to the synthesis of the respective fine chemical 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid its function as a probe extends to the detection of microorganisms, plant tissues, plants, plant variets, plant ecotypes or plant genera with varying capability or potential for synthesis of the respective fine chemical 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid. Therefore in one embodiment the present invention relates to a method for analyzing the capability or potential of a plant tissue, a plant, a plant variety or plant ecotype to produce the fine chemical 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid by using the nucleic acid of the invention or parts thereof as a probe to detect the amount of the nucleic acid of the invention in the non-human organism or a part thereof in comparison to another non-human organism.

The nucleic acid molecule of the invention encodes a polypeptide or portion thereof which includes an amino acid sequence which is sufficiently homologous to the amino acid sequence shown in respective line in Table II, application no. 12, columns 5 or 8 such that the protein or portion thereof maintains the ability to confer the production or the increased production of the respective fine chemical 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid as compared to a corresponding, e.g. non-transformed, wild type non-human organism, like a microorganism or a plant cell, plant or part thereof, in particular increasing the activity as mentioned above or as described in the examples in microorganisms or plants.

As used herein, the language “sufficiently homologous” refers to proteins or portions thereof which have amino acid sequences which include a minimum number of identical or equivalent amino acids (e.g. an amino acid which has a similar side chain as an amino acid in one of the sequences of the polypeptide of the present invention) to an amino acid sequence shown in the respective line in Table II, application no. 12, columns 5 or 8 such that the protein or portion thereof is able to confer the production or the increased production of the respective fine chemical 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid as compared to a corresponding, e.g. non-transformed, wild type non-human organism, like a microorganism or a plant cell, plant or part thereof. For example having the activity of a protein as shown in the respective line in Table II, application no. 12, column 3 and as described herein.

In an embodiment of the present invention, the nucleic acid molecule of the present invention comprises a nucleic acid molecule that encodes a portion of the protein of the present invention. The protein is at least about 30%, 35%, 40%, 45% 50%, 55%, 60%, 65%, 70%, 75%, 80%, 85%, 90%, 91%, 92%, 93%, 94%, 95%, 96%, 97%, 98%, 99%, 99.5% or more homologous to an entire amino acid sequence shown in the respective line of Table II, application no. 12, column 5 or 8 and has the above-mentioned activity, e.g. conferring the production or the increased production of the respective fine chemical 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid, respectively, as compared to a corresponding, e.g. non-transformed, wild type non-human organism, like a microorganism or a plant cell, plant or part thereof by, for example in a embodiment expression either in the cytosol or in an organelle such as a plastid or mitochondria or both, preferably in a plastid, or in another embodiment by targeted or non-targeted expression.

[0338.1.8.12] to [0339.1.8.12] for the disclosure of these paragraph see [0338.1.8.8] to [0339.1.8.12] above.

The invention further relates to nucleic acid molecules or methods using said nucleic acid molecules that differ from one of the nucleotide sequences shown in the respective line in Table I, application no. 12, columns 5 or 8, preferably the coding region thereof, or fragments or homologs thereof, due to degeneracy of the genetic code but encode a polypeptide of the present invention, in particular a polypeptide having the above-mentioned activity, e.g. as those polypeptides depicted in the respective line in Table II, application no. 12, columns 5 or 8, fragments or homologs thereof. Advantageously, the nucleic acid molecule of the invention comprises, or in an other embodiment has, a nucleotide sequence encoding a protein comprising, or in an other embodiment having, an amino acid sequence shown in the respective line in Table II, application no. 12, columns 5 or 8, fragments or homologs thereof. In a further embodiment, the nucleic acid molecule of the invention encodes a full length protein which is substantially homologous to an amino acid sequence shown in the respective line in Table II, application no. 12, columns 5 or 8, or homologs thereof. However, in an embodiment, the nucleic acid molecule of the present invention does not consist of the sequence shown in the respective line in Table I, preferably Table IA, application no. 12, columns 5 or 8, preferably the coding region thereof, but the functional homologs thereof.

[0341.1.8.12] to [0343.1.8.12] for the disclosure of these paragraphs see [0341.1.8.8] to [0343.1.8.8] above.

Accordingly, in another embodiment, a nucleic acid molecule of the invention is at least 15, 20, 25 or 30 nucleotides in length. Preferably, it hybridizes under stringent conditions to a nucleic acid molecule comprising a nucleotide sequence of the nucleic acid molecule of the present invention or used in the process of the present invention, e.g. comprising the sequence shown in the respective line in Table I, application no. 12, columns 5 or 8, preferably the coding region thereof. In another embodiment the nucleic acid molecule is preferably at least 20, 30, 50, 100, 250 or more nucleotides in length.

for the disclosure of this paragraph see [0345.1.8.8] above.

Preferably, a nucleic acid molecule of the invention that hybridizes under stringent conditions to a sequence shown in the respective line in Table I, application no. 12, columns 5 or 8, preferably the coding region thereof, corresponds to a naturally-occurring nucleic acid molecule of the invention. As used herein, a “naturally-occurring” nucleic acid molecule refers to a RNA or DNA molecule having a nucleotide sequence that occurs in nature (e.g. encodes a natural protein). Preferably, the nucleic acid molecule encodes a natural protein having above-mentioned activity, e.g. conferring the production or the increased production of the respective fine chemical 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid, respectively, as compared to a corresponding, e.g. non-transformed, wild type non-human organism, like a microorganism or a plant cell, plant or part thereof after increasing the expression or activity thereof or the activity of a protein of the invention or used in the process of the invention, in an embodiment for example expression either in the cytosol or in an organelle such as a plastid or mitochondria, preferably in plastids, or, in another embodiment by targeted or non-targeted expression.

for the disclosure of this paragraph see [0347.1.8.8] above.

For example, nucleotide substitutions leading to amino acid substitutions at “non-essential” amino acid residues can be made in a sequence of the nucleic acid molecule of the invention or used in the process of the invention, e.g. shown in the respective line in Table I, application no. 12, columns 5 or 8, preferably the coding region thereof, or fragments or homologs thereof.

[0349.1.8.12] to [0350.1.8.12] for the disclosure of these paragraphs see [0349.1.8.8] to [0350.1.8.8] above.

Accordingly, the invention relates to nucleic acid molecules encoding a polypeptide having above-mentioned activity, in a non-human organisms or parts thereof by for example expression either in the cytosol or in an organelle such as a plastid or mitochondria or both, preferably in plastids, that contain changes in amino acid residues that are not essential for said activity. Such polypeptides differ in amino acid sequence from a sequence depicted in the respective line in Table II, especially Table IIA, application no. 12, columns 5 or 8, but retain said activity described herein. The nucleic acid molecule can comprise a nucleotide sequence encoding a polypeptide, wherein the polypeptide comprises an amino acid sequence at least about 50% identical to an amino acid sequence shown in the respective line in Table II, application no. 12, columns 5 or 8, and is capable of conferring the production or the increased production of the respective fine chemical 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid, respectively, as compared to a corresponding, e.g. non-transformed, wild type non-human organism, like a microorganism or a plant cell, plant or part thereof, after increasing its activity for example in an embodiment by expression either in the cytosol or in an organelle such as a plastid or mitochondria or both, preferably in plastids, or, in another embodiment by targeted or non-targeted expression. Preferably, the protein encoded by the nucleic acid molecule is at least about 60%, 70%, 80%, 90%, 95%, 96%, 97%, 98%, 99% or 99.5% identical to the sequence shown in the respective line in Table II, application no. 12, columns 5 or 8.

[0352.1.8.12] to [0357.1.8.12] for the disclosure of these paragraphs see [0352.1.8.8] to [0357.1.8.8] above.

Functional equivalents of the nucleic acid molecules according to the present invention and/or used in the process according to the present invention represent an embodiment of homologs. Functional equivalents derived from the nucleic acid molecules as shown in the respective line in Table I, application no. 12, columns 5 or 8, preferably the coding region thereof, according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the nucleic acid molecules as shown in the respective line in Table I, application no. 12, columns 5 or 8, preferably the coding region thereof, according to the invention and encode polypeptides having essentially the same properties as the polypeptide as shown in the respective line in Table II, application no. 12, columns 5 or 8.

Functional equivalents derived from one of the polypeptides as shown in the respective line in Table II, application no. 12, columns 5 or 8 according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the polypeptides as shown in the respective line in Table II, application no. 12, columns 5 or 8 according to the invention and having essentially the same properties as the polypeptide as shown in the respective line in Table II, application no. 12, columns 5 or 8.

for the disclosure of this paragraph see [0359.1.8.8] above.

A nucleic acid molecule encoding a homolog to a polypeptide depicted in the respective line in Table II, application no. 12, columns 5 or 8 can be created by introducing one or more nucleotide substitutions, additions or deletions into the nucleic acid molecule of the present invention, in particular of Table I, application no. 12, columns 5 or 8, in the respective line, such that one or more amino acid substitutions, additions or deletions are introduced into the encoded protein. Mutations can be introduced into the nucleic acid molecules as depicted in the respective line in Table I, application no. 12, columns 5 or 8, preferably the coding region thereof, by standard techniques, such as site-directed mutagenesis, PCR-mediated mutagenesis or other methods known to the person skilled in the art.

[0361.1.8.12] to [0363.1.8.12] for the disclosure of these paragraphs see [0361.1.8.8] to [0363.1.8.8] above.

Homologues of the nucleic acid sequences used, with the sequences shown in the respective line in Table I, application no. 12, columns 5 or 8, preferably the coding region thereof, comprise also allelic variants with at least approximately 30%, 35%, 40%, 45%, 50%, 55%, 60%, 65%, 70%, 75%, 80%, 85%, 90%, 91%, 92%, 93%, 94% 95%. 96%, 97%, 98%, 99%, 99.5% or more % homology with one of the nucleotide sequences shown or the abovementioned derived nucleic acid sequences or their homologues or derivatives or parts of these. Allelic variants encompass in particular functional variants which can be obtained by deletion, insertion or substitution of nucleotides from the sequences shown in the respective line, preferably in Table I, columns 5 or 8, preferably the coding region thereof, or from the derived nucleic acid sequences, the intention being, however, that the enzymatic activity or the biological activity of the resulting proteins synthesized is advantageously retained or increased.

In one embodiment of the present invention, the nucleic acid molecule of the invention or used in the process of the invention comprises the sequences shown in the respective line in any of the Table I, application no. 12, columns 5 or 8, preferably the coding region thereof, fragments or homologs thereof. It is preferred that the nucleic acid molecule comprises as little as possible other nucleotides not shown in the respective line in any one of Table I, application no. 12, columns 5 or 8, preferably the coding region thereof, fragments or homologs thereof. In one embodiment, the nucleic acid molecule comprises less than 500, 400, 300, 200, 100, 90, 80, 70, 60, 50 or 40 further nucleotides. In a further embodiment, the nucleic acid molecule comprises less than 30, 20 or 10 further nucleotides. In one embodiment, the nucleic acid molecule used in the process of the invention is identical to the sequences shown in the respective line in Table I, application no. 12, columns 5 or 8, preferably the coding region thereof.

Also preferred is that the nucleic acid molecule used in the process of the invention encodes a polypeptide comprising the sequence shown in the respective line in Table II, application no. 12, columns 5 or 8, fragments or homologs thereof. In one embodiment, the nucleic acid molecule encodes less than 150, 130, 100, 80, 60, 50, 40 or 30 further amino acids. In a further embodiment, the encoded polypeptide comprises less than 20, 15, 10, 9, 8, 7, 6 or 5 further amino acids. In one embodiment used in the inventive process, the encoded polypeptide is identical to the sequences shown in the respective line in Table II, application no. 12, columns 5 or 8.

In one embodiment, the nucleic acid molecule of the invention or used in the process encodes a polypeptide comprising the sequence shown in the respective line in Table II, application no. 12, columns 5 or 8, or fragments or homologs thereof, and comprises less than 100 further nucleotides. In a further embodiment, said nucleic acid molecule comprises less than 30 further nucleotides. In one embodiment, the nucleic acid molecule used in the process is identical to a coding sequence of the sequences shown in the respective line in Table I, application no. 12, columns 5 or 8.

Polypeptides (=proteins), which still have the essential biological or enzymatic activity of the polypeptide of the present invention conferring the production or the increased production of the respective fine chemical 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid, respectively, as compared to a corresponding, e.g. non-transformed, wild type non-human organism, like a microorganism or a plant cell, plant or part thereof, i.e. whose activity is essentially not reduced, are polypeptides with at least 10%, 20%, 30%, 40%, 50%, 60%, 70%, 80%, 90%, 95% or more of the wild type biological activity or enzymatic activity, advantageously, the activity is essentially not reduced in comparison with the activity of a polypeptide shown in the respective line in Table II, application no. 12, columns 5 or 8 expressed under identical conditions.

Homologues of nucleic acid molecules shown in the respective line in Table I, application no. 12, columns 5 or 8, preferably the coding region thereof, or fragments thereof, or of the derived sequences shown in the respective line in Table II, application no. 12, columns 5 or 8, or fragments thereof, also mean truncated sequences, cDNA, single-stranded DNA or RNA of the coding and noncoding DNA sequence. Homologues of said sequences are also understood as meaning derivatives, which comprise noncoding regions such as, for example, UTRs, introns, terminators, enhancers or promoter variants. The promoters upstream of the nucleotide sequences stated can be modified by one or more nucleotide substitution(s), insertion(s) and/or deletion(s) without, however, interfering with the functionality or activity either of the promoters, the open reading frame (=ORF) or with the 3′-regulatory region such as terminators or other 3′-regulatory regions, which are far away from the ORF. It is furthermore possible that the activity of the promoters is increased by modification of their sequence, or that they are replaced completely by more active promoters, even promoters from heterologous organisms. Appropriate promoters are known to the person skilled in the art and are mentioned herein below.

[0370.1.8.12] to [0379.1.8.12] for the disclosure of these paragraphs see [0370.1.8.8] to [0379.1.8.12] above.

Moreover, a native polypeptide conferring the increase of the respective fine chemical 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid, respectively, in a non-human organism or a part thereof can be isolated from cells (e.g., endothelial cells), for example using the antibody of the present invention as described below, in particular, an antibody against proteins having 2,3-dihydroxyphenylpropionate 1,2-dioxygenase, 2-C-methyl-D-erythritol 4-phosphate cytidylyltransferase, 30S ribosomal protein S3, 3-ketoacyl-CoA thiolase, 3-oxoacyl-[acyl-carrier-protein] reductase, 3-oxoacyl-[acyl-carrier-protein] synthase, 40S ribosomal protein S17, ABC transporter component, ABC transporter permease protein, acetyl-coenzyme A synthetase, acid shock protein, acyl-CoA synthase, acyl-desaturase, adenosine kinase, aldolase, alkaline phosphatase isozyme conversion protein, amino acid ABC transporter permease protein, aminotransferase, ankyrin repeat protein, At2g33510-protein, ATPase epsilon subunit, ATP-binding component of a transport system, b0050-protein, b0249-protein, b0371-protein, b0518-protein, b0801-protein, b1046-protein, b1346-protein, b1470-protein, b1522-protein, b1670-protein, b1703-protein, b1837-protein, b2032-protein, b2099-protein, b2107-protein, b2126-protein, b2139-protein, b2360-protein, b2474-protein, b2513-protein, b2654-protein, b3442-protein, b3644-protein, b3812-protein, b3944-protein, b4023-protein, b4205-protein, beta-hydroxylase, betaine aldehyde dehydrogenase, bifunctional protein (phosphoribosyltransferase and regulatory protein), bifunctional purine biosynthesis protein, binding-protein-dependent transport systems inner membrane component, biotin synthase, calcium-dependent protein kinase, carbon dioxide concentrating mechanism protein, cardiolipin synthase, CDP-diacylglycerol-serine O-phosphatidyltransferase, cell division control protein, cell wall protein, chlorophyllase, citrate synthase, coproporphyrinogen III oxidase, cyclin, cystathionine gamma-synthase, cysteine synthase A, dihydrolipoamide dehydrogenase, DNA mismatch repair protein, DnaJ-like chaperone, electron transport complex protein, Elongation factor Tu, ethanolamine utilization protein, F-box protein, ferredoxin, fructose 1,6-bisphosphatase, fumarylacetoacetate hydrolase, glutamate 5-kinase, glutamate-ammonia-ligase, glutaredoxin, glycerol-3-phosphate dehydrogenase, glycine cleavage system H protein, GM02LC38418-protein, heat shock protein, heat shock transcription factor, HesB/YadR/YfhF family protein, homocitrate synthase, hydrolase, isocitrate dehydrogenase, lipoprotein precursor, malate dehydrogenase, malonyl CoA-acyl carrier protein transacylase, metal-dependent hydrolase, molecular chaperone, molecular chaperone portein, monothiol glutaredoxin, multiple antibiotic resistance protein, murein transglycosylase, N-acetyl-gamma-glutamyl-phosphate reductase, NADP-dependent malic enzyme, outer-membrane lipoprotein precursor, oxidoreductase, permease protein of ABC transporter, phage-related repressor protein, phosphatase, phosphate import ATP-binding protein, phosphatidylserine synthase, phospho-2-dehydro-3-deoxyheptonate aldolase, phosphoadenosine phosphosulfate reductase, phosphotransferase, phosphotransferase system component, polygalacturonase, protein kinase, protein phosphatase, purine nucleoside phosphorylase, pyridoxamine kinase, recombinase A, sec-independent protein translocase, Sec-independent protein translocase subunit, sensor histidine kinase, sensor kinase, sll0281-protein, sll0406-protein, small nuclear ribonucleoprotein , sodium/proton antiporter, squalene monooxygenase, sugar transport system permease protein, threonine dehydratase, transcription factor, transcriptional regulator, transcriptional regulator protein, transport protein, tryptophan synthase alpha chain, TTC1386-protein, vacuolar processing enzyme, ycl027w-protein, ycl049c-protein, ydr273w-protein, ydr339c-protein, YFL019C-protein, ygl237c-protein, YGR104C-protein, yhr033w-protein, YJL127W-A-protein, ylr326w-protein, yml083c-protein, yml084w-protein, or ynl321w-protein activity, respectively, or an antibody against polypeptides as shown in the respective line in Table II, application no. 12, columns 5 or 8, or fragments or homologs thereof which can be produced by standard techniques utilizing the polypeptid of the present invention or fragment thereof, i.e., the polypeptide of this invention (FCRP). Preferred are monoclonal antibodies.

for the disclosure of this paragraph see [0381.1.8.8] above.

In one embodiment, the present invention relates to a polypeptide as depicted in the respective line in Table II, especially in Table IIA or especially in Table IIB, application no. 12, columns 5 or 8, or fragment or homolog thereof or as coded by the nucleic acid molecule shown in the respective line in Table I, especially Table IA or especially Table IB, application no. 12, columns 5 or 8, preferably the coding region thereof, or fragment or homolog thereof.

In an embodiment, the present invention relates to a polypeptide comprising or consisting of a consensus sequence shown in the respective line in Table IV, application no. 12, column 8 or at least one polypeptide motif shown in the respective line in Table IV, application no. 12, column 8.

In an embodiment thereof, the present invention relates to a polypeptide comprising or consisting of a consensus sequence shown in the respective line in Table IV, application no. 12, column 8 or comprising at least one polypeptide motif shown in the respective line in Table IV, application no. 12, column 8, whereby not more than 20, 15, 10, 9, 8, 7, 6, 5, 4, 3, 2, or 1, or 0 of the amino acids can be replaced by any amino acid.

In another embodiment thereof, the present invention relates to a polypeptide comprising or consisting of a consensus sequence shown in the respective line in Table IV, application no. 12, column 8 or comprising at least one polypeptide motif shown in the respective line in Table IV, application no. 12, column 8, whereby not more than 15%, 10%, 5%, 4%, 3%, 2% or1% or 0% of the amino acids defined as distinct specific amino acids are/is replaced by another amino acid.

In another embodiment thereof, the present invention relates to a polypeptide comprising or consisting of a consensus sequence shown in the respective line in Table IV, application no. 12, column 8 or comprising at least one polypeptide motif shown in the respective line in Table IV, application no. 12, column 8, whereby not more than 20, 15, 10, 9, 8, 7, 6, 5, 4, 3, 2, or 1, or 0 amino acids are inserted into the consensus sequence or polypeptide motif.

In an embodiment of the present invention, the polypeptide of the present invention comprises or consists of a consensus sequence or at least one polypeptide motif shown in the respective line in Table IV, application no. 12, column 8, whereby said polypeptide distinguishes over the sequence depicted in the respective line in Table II, application no. 12, columns 5 or 8 by one or more amino acids. In one embodiment, said polypeptide of the invention distinguishes over the sequence shown in the respective line in Table II, application no. 12, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 12, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in the respective line in Table II, application no. 12, columns 5 and 8.

In an embodiment, the invention relates to a polypeptide conferring an increase in the respective fine chemical 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid, respectively, in a non-human organism, especially a microorganism or a plant, or a part thereof, being encoded by the nucleic acid molecule of the invention or used in the process of the invention and having a sequence which distinguishes over the sequence as shown in the respective line in Table II, application no. 12, columns 5 or 8 by one or more amino acids (but not exceeding 5%, preferably 4%, 3%, 2%, 1%, 0.5%, 0.2% or 0.1%). In an embodiment, said polypeptide of the invention does not comprise or consist of the sequence shown in the respective line in Table II, application no. 12, columns 5 or 8.

In an embodiment, said polypeptide of the present invention is less than 100%, 99.999%, 99.99%, 99.9% or 99% identical. In one embodiment, said polypeptide which differs at least in one or more amino acids (but not exceeding 5%, preferably 4%, 3%, 2%, 1%, 0.5%, 0.2% or 0.1%) from the polypeptide shown in the respective line in Table II, application no. 12, columns 5 and 8 does not comprise a protein of the sequence shown in the respective line in Table II A and/or II B, application no. 12, columns 5 or 8.

In an embodiment, the present invention relates to a polypeptide having the activity of the protein as shown in the respective line in Table II, application no. 12, column 3, which distinguishes over the sequence depicted in the respective line in Table II, application no. 12, columns 5 or 8 by one or more amino acids, preferably by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 12, columns 5 and 8 by not more than 80%, 70%, 60%, 50%, 40%, 30%, 20%, 10%, 5%, 4%, 3%, 2%, 1% or 0.5%.

In another embodiment the polypeptide of the invention takes the form of a preprotein consisting of a plastidial or mitochondrial transit peptide joint to a polypeptide having the activity of the protein as shown in Table II, column 3, from which the transit peptide is preferably cleaved off upon transport of the preprotein into the organelle for example into the plastid or mitochondrion.

In another embodiment the polypeptide of the invention takes not the form of a preprotein.

[0390.1.8.12] to [0391.1.8.12] for the disclosure of these paragraphs see [0390.1.8.8] to [0391.1.8.8] above.

A polypeptide of the invention can participate in the process of the present invention. The polypeptide or a portion thereof comprises preferably an amino acid sequence, which is sufficiently homologous to an amino acid sequence shown in the respective line in Table II A and/or II B, application no. 12, columns 5 or 8.

Further, the polypeptide can have an amino acid sequence which is encoded by a nucleotide sequence which hybridizes, preferably hybridizes under stringent conditions as described above, to a nucleotide sequence of the nucleic acid molecule of the present invention. Accordingly, the polypeptide has an amino acid sequence which is encoded by a nucleotide sequence that is at least about 35%, 40%, 45%, 50%, 55%, 60%, 65%, 70%, 75%, 80%, 85%, 90%, 91%, 92%, 93%, 94%, 95%, 96%, 97%, 98%, 99%, 99.5% or more homologous to one of the amino acid sequences as shown in the respective line in Table II A and/or II B, application no. 12, columns 5 or 8, or fragments thereof. The preferred polypeptide of the present invention preferably possesses at least one of the activities according to the invention and described herein. A preferred polypeptide of the present invention includes an amino acid sequence encoded by a nucleotide sequence which hybridizes, preferably hybridizes under stringent conditions, to a nucleotide sequence shown in the respective line in Table I A and/or I B, application no. 12, columns 5 or 8, preferably the coding region thereof, or fragments thereto or which is homologous thereto, as defined above.

The polypeptide of the present invention can vary from the sequences shown in the respective line in Table II A and/or II B, application no. 12, columns 5 or 8 in amino acid sequence due to natural variation or mutagenesis, as described in detail herein. Accordingly, the polypeptide comprise an amino acid sequence which is at least about 35%, 40%, 45%, 50%, 55%, 60%, 65%, 70%, 75%, 80%, 85% or 90, 91%, 92%, 93%, 94%, 95%, 96%, 97%, 98%, 99%, 99.5% or more homologous to an entire amino acid sequence shown in the respective line in Table II A and/or II B, application no. 12, columns 5 or 8.

Biologically active portions of a polypeptide of the present invention include peptides comprising amino acid sequences derived from the amino acid sequence of the polypeptide of the present invention or used in the process of the present invention, e.g., the amino acid sequence shown in the respective line in Table II, application no. 12, columns 5 or 8 or the amino acid sequence of a protein homologous thereto, which include fewer amino acids than a full length polypeptide of the present invention or used in the process of the present invention or the full length protein which is homologous to a polypeptide of the present invention or used in the process of the present invention depicted herein, and exhibit at least one activity of a polypeptide of the present invention or used in the process of the present invention.

for the disclosure of this paragraph see [0396.1.8.8] above.

Manipulation of the nucleic acid molecule of the invention may result in the production of a protein having differences from the protein as shown in the respective line in Table II, application no. 12, column 5 or 8. Differences shall mean at least one amino acid different from the sequences as shown in the respective line in Table II, application no. 12, column 5 or 8, preferably at least 2, 3, 4, 5, 6, 7, 8, 9 or 10 amino acids (especially but not exceeding 0.1%, preferably 0.2%, 0.5%, 1%, 2%, 3% 4% or 5%), more preferably at least 15, 20, 25, 30, 35, 40, 45 or 50 amino acids (especially but not exceeding 0.5%, preferably 1%, 2%, 3% 4%, 5%, 10% or 25%) different from the sequences as shown in the respective line in Table II, application no. 12, column 5 or 8. These proteins may be improved in efficiency or activity, or may be more stable and therefore present in greater numbers in the cell as compared to the wild-type cell.

Any mutagenesis strategies for the polypeptide of the present invention or the polypeptide used in the process of the present invention to result in increasing said activity are not meant to be limiting; variations on these strategies will be readily apparent to one skilled in the art. Using such strategies, and incorporating the mechanisms disclosed herein, the nucleic acid molecule and polypeptide of the invention may be utilized to generate algae, ciliates, fungi, other microorganisms like C. glutamicum or plants or parts thereof, expressing proteins as shown in the respective line in Table II, application no. 12, column 5 or 8, or mutated proteins thereof. The nucleic acid molecules and polypeptide molecules of the invention are expressed such that the yield, production, and/or efficiency of production of a desired compound is improved.

[0399.1.8.12] to [0401.1.8.12] for the disclosure of these paragraphs see [0399.1.8.8] to [0401.1.8.8] above.

In one embodiment, a protein (=polypeptide) as shown in the respective line in Table II, application no. 12, column 5 or 8 refers to a polypeptide having an amino acid sequence corresponding to the polypeptide of the invention, especially to a polypeptide as shown in Table II, application no. 12, columns 5 or 8 or homologs thereof, or used in the process of the invention, whereas a “non-inventive protein or polypeptide” or “other polypeptide” refers to a polypeptide having an amino acid sequence corresponding to a protein which is not substantially homologous to a polypeptide of the invention, preferably which is not substantially homologous to a polypeptide or protein as shown in the respective line in Table II, application no. 12, column 5 or 8 or homologs thereof, e.g., a protein which does not confer the activity described herein and which is derived from the same or a different organism.

[0403.1.8.12] to [0409.1.8.12] for the disclosure of these paragraphs see [0403.1.8.8] to [0409.1.8.8] above.

As the nucleic acid molecules of the invention and/or the expression thereof is related to the synthesis of the respective fine chemical 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid its function as a probe extends to the detection of microorganisms, plant tissues, plants, plant variets, plant ecotypes or plant genera with varying, advantageously increased, capability or potential for synthesis of the respective fine chemical 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid. Therefore in one embodiment the present invention relates to a method for analyzing the capability or potential of a plant tissue, a plant, a plant variety or ecotype to produce the respective fine chemical 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid by using the respective antibody of the invention as a probe to detect the amount of the polypeptide encoded by said nucleic acid molecule of the invention in a non-human organism in comparison to another organism.

Therefore in one embodiment the invention relates to a method to probe a non-human organism for its capability for the production of the respective fine chemical comprising the steps of
a) analyzing the sequence and/or the expression of the respective nucleic acid molecule of the invention in least two non-human organisms which differ in the capability to produce the respective fine chemical;
b) associate the different sequence and/or expression level of the nucleic acid molecules of the invention with their different capability to produce the respective fine chemical;
c) analyze the sequence and/or the expression of the nucleic acid molecule of the invention in at least a third non-human organism;
d) predict the capability of the at least third organism to produce the respective fine chemical based by comparing the sequence and/or expression of the nucleic acid of the invention to the sequences and/or expression levels of the nucleic acid molecule of the invention of the at least two organisms analyzed in step a.

[0411.1.8.12] to [0430.1.8.12] for the disclosure of these paragraphs see [0411.1.8.8] to [0430.1.8.8] above.

Accordingly the present invention relates to any cell transgenic for any nucleic acid characterized as part of the invention, e.g. conferring the increase of the respective fine chemical 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid in a cell or a non-human organism or a part thereof, e.g. the nucleic acid molecule of the invention, the nucleic acid construct of the invention, the vector of the invention, the expression cassette according to the invention, or a nucleic acid molecule encoding the polypeptide of the invention, e.g. encoding a polypeptide having an activity as the protein as shown in the respective line in Table II, application no. 12, column 3. Due to the above-mentioned activity the respective fine chemical 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid content in a cell or a non-human organism is increased. For example, due to modulation or manipulation, the cellular activity is increased, in a preferred embodiment in organelles such as plastids or mitochondria, e.g. due to an increased expression or specific activity or specific targeting of the subject matters of the invention in a cell or a non-human organism or a part thereof especially in organelles such as plastids or mitochondria, or in another embodiment in the cytosol. Transgenic for a polypeptide having a protein or a protein activity means herein that due to modulation or manipulation of the genome, the activity of protein as shown in the respective line in Table II, application no. 12, column 3 or a protein as shown in the respective line in Table II, application no. 12, column 3-like activity is increased in the cell or non-human organism or part thereof, especially in organelles such as plastids or mitochondria, or especially in the cytosol. Examples are described above in context with the process of the invention.

for the disclosure of this paragraph see [0432.1.8.8] above.

A naturally occurring expression cassette—for example the naturally occurring combination of the promoter of the gene encoding a protein as shown in the respective line in Table II, application no. 12, column 5 or 8, preferably the coding region thereof, or fragments or homologs thereof with the corresponding encoding gene—becomes a transgenic expression cassette when it is modified by non-natural, synthetic “artificial” methods such as, for example, mutagenesis. Such methods have been described (U.S. Pat. No. 5,565,350; WO 00/15815; also see above).

[0434.1.8.12] to [0435.1.8.12] for the disclosure of these paragraphs see [0434.1.8.8] to [0435.1.8.8] above.

Transgenic plants comprising the respective fine chemical(s) synthesized in the process according to the invention can be marketed directly without isolation of the fine chemical synthesized. In the process according to the invention, plants are understood as meaning all plant parts, plant organs such as leaf, stalk, root, tubers or seeds or propagation material or harvested material or the intact plant. In this context, the seed encompasses all parts of the seed such as the seed coats, epidermal cells, seed cells, endosperm or embryonic tissue. The fine chemical produced in the process according to the invention may, however, also be isolated from the plant. In case of 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid, respectively, this can be in free form or bound to proteins. Fine chemical(s) produced by this process can be harvested by harvesting the non-human organisms either from the culture in which they grow or from the field. For example, this can be done via squeezing, grinding and/or extraction, salt precipitation and/or ion-exchange chromatography of the plant parts, preferably the plant seeds, plant fruits, plant tubers and the like.

[0437.1.8.12] to [0454.1.8.12] for the disclosure of these paragraphs see [0437.1.8.8] to [0454.1.8.8] above.

In one embodiment, the present invention relates to a method for the identification of a gene product conferring the production of or an increase in the fine chemical production in a non-human cell, comprising the following steps:
(a) contacting, e.g. hybridizing, a, some or all nucleic acid molecules of a sample, e.g. cells, tissues, plants or microorganisms or a nucleic acid library, which can contain a candidate gene encoding a gene product conferring the production of or an increase in the fine chemical after expression, with the nucleic acid molecule of the present invention as shown in the respective line in column 5 or 8 of Table I A or B of application no. 12, preferably the coding region thereof, or a fragment or homolog thereof;
(b) identifying the nucleic acid molecules, which hybridize under relaxed stringent conditions with the nucleic acid molecule of the present invention in particular to the nucleic acid molecule sequence shown in the respective line in Table I, application no. 12, columns 5 or 8, preferably the coding region thereof, of a fragment or a homolog thereof, preferably in Table I B, application no. 12, columns 5 or 8, preferably the coding region thereof, of a fragment or a homolog thereof, and, optionally, isolating the full length cDNA clone or complete genomic clone;
(c) introducing the candidate nucleic acid molecules or a fragment thereof in host cells, preferably in a plant cell or a microorganism, appropriate for producing the fine chemical;
(d) expressing the identified nucleic acid molecules in the host cells for which the production of or the increased production of the fine chemical is desired;
(e) assaying the fine chemical level in the non-human host cells; and
(f) identifying the nucleic acid molecule and/or its gene product which expression confers the production of or an increase in the fine chemical level in the non-human host cell after expression compared to the wild type.

for the disclosure of this paragraph see [0456.1.8.8] above.

In another embodiment, the present invention relates to a method for the identification of a gene product conferring the production of or an increase in the fine chemical production in a cell, comprising the following steps:
(a) identifying a nucleic acid molecule of a non-human organism, which is at least 20%, 25%, 30%, 35%, 40%, 50%, 60%, 70%, 80%, 90%, 95%, 96%, 97%, 98%, 99%, 99.5% or more homolog to the nucleic acid molecule encoding a polypeptide comprising the polypeptide molecule as shown in the respective line in column 5 or 8 of Table II, application no. 12, or comprising a consensus sequence or a polypeptide motif as shown in the respective line in column 8 of Table IV, application no. 12, or being encoded by a nucleic acid molecule comprising a polynucleotide as shown in the respective line in column 5 or 8 of Table I, application no. 12, preferably the coding region thereof, or a homologue thereof as described herein, for example via homology search in a data bank;
(b) introducing the candidate nucleic acid molecule in non-human host cells, preferably in a plant cell or a microorganism, appropriate for producing the fine chemical;
(c) expressing the identified nucleic acid molecule in the non-human host cells;
(d) assaying the fine chemical level in the non-human host cells; and
(e) identifying the nucleic acid molecule and its gene product which expression confers the production of or an increase in the fine chemical level in the non-human host cell after expression compared to the wild type.

for the disclosure of this paragraph see [0458.1.8.8] above.

Further, the nucleic acid molecules disclosed herein, in particular the nucleic acid molecules shown in the respective line in column 5 or 8 of Table I A or B of application no. 12, may be sufficiently homologous to the sequences of related species such that these nucleic acid molecules may serve as markers for the construction of a genomic map in related organism or for association mapping. Furthermore natural variation in the genomic regions corresponding to nucleic acids disclosed herein, in particular the nucleic acid molecule shown in the respective line in column 5 or 8 of Table I A or B, of application no. 12 or homologous thereof, may lead to variation in the activity of the proteins disclosed herein, in particular the proteins comprising polypeptides as shown in the respective lines in column 5 or 8 of Table II A or B, of application no. 12 or their homologous, or comprising the consensus sequence or the polypeptide motif as shown in the respective line in column 8 of Table IV, and in consequence in a natural variation of an increased fine chemical production.

In consequence natural variation eventually also exists in form of more active allelic variants leading already to a relative increase in the fine chemical. Different variants of the nucleic acid molecules disclosed herein, in particular the nucleic acid molecules comprising the nucleic acid molecules as shown in the respective line in column 5 or 8 of Table I A or B of application no. 12, preferably the coding region thereof, which corresponds to different levels of increase in fine chemical, e.g. different levels of increase in fine chemical, can be indentified and used for marker assisted breeding for an enhanced production of the fine chemical.

Natural variation may also exist in the regulatory regions, e.g. the promoters of the natural genes comprising the nucleic acid molecules as shown in the respective line column 5 or 8 of Table I A or B of application no. 12, preferably the coding region thereof, which can similarly correspond to different levels of increase in the fine chemical, e.g. different levels of increase in the fine chemical can be identified and used for marker assisted breeding for an enhanced production of the fine chemical.

Accordingly, the present invention relates to a method for breeding plants for the production of the fine chemical, comprising
(a) selecting a first plant variety with an increased production of the fine chemical, e.g. based on an increased expression of a nucleic acid molecule of the invention as disclosed herein, in particular of a nucleic acid molecule comprising a nucleic acid molecule as shown in the respective line in column 5 or 8 of Table I A or B of application no. 12, preferably the coding region thereof, or fragments or homologs thereof, or a polypeptide comprising a polypeptide as shown in the respective line in column 5 or 8 of Table II A or B of application no. 12, or fragments or homologs thereof, or comprising a consensus sequence or a polypeptide motif as shown in the respective line in column 8 of Table IV, or a homolog thereof, as described herein;
(b) associating the level of increased production of the fine chemical, with the expression level or the genomic structure of a gene encoding said polypeptide or said nucleic acid molecule;
(c) crossing the first plant variety with a second plant variety, which significantly differs in its level of fine chemical; and
(d) identifying, which of the offspring varieties has got increased production of the fine chemical by the expression level of said polypeptide or nucleic acid molecule or the genomic structure of the gene(s) encoding said polypeptide or nucleic acid molecule of the invention.

[0462.1.8.12] to [0482.1.8.12] for the disclosure of these paragraphs see [0462.1.8.8] to [0482.1.8.8] above.

A special embodiment of this invention is related to genes which increase or generate the production of the fine chemical oleic acid on the one hand and of 2-hydroxy-behenic acid, 2-hydroxytetracosenic acid (preferably 2-hydroxynervonic acid), behenic acid, cerebronic acid and/or nervonic acid on the other hand in plant cells, plants or part thereof. Phenotypes thereto are associated with yield of plants (=yield related phenotypes). In accordance with the invention, therefore, the respective genes identified in Table I, wherein in column 7 oleic acid, 2-hydroxybehenic acid, 2-hydroxytetracosenic acid (preferably 2-hydroxynervonic acid), behenic acid, cerebronic acid or nervonic acid is mentioned, especially the coding region thereof, or homologs or fragments thereof, may be employed to enhance any yield-related phenotype. Increased yield may be determined in field trials of transgenic plants and suitable control plants. Alternatively, a transgene's ability to increase yield may be determined in a model plant. An increased yield phenotype may be determined in the field test or in a model plant by measuring any one or any combination of the following phenotypes, in comparison to a control plant: yield of dry harvestable parts of the plant, yield of dry aerial harvestable parts of the plant, yield of underground dry harvestable parts of the plant, yield of fresh weight harvestable parts of the plant, yield of aerial fresh weight harvestable parts of the plant yield of underground fresh weight harvestable parts of the plant, yield of the plant's fruit (both fresh and dried), grain dry weight, yield of seeds (both fresh and dry), and the like.

The most basic yield-related phenotype is increased yield associated with the presence of the gene or a homolog or a fragment thereof as a transgene in the plant, i.e., the intrinsic yield of the plant. Intrinsic yield capacity of a plant can be, for example, manifested in a field test or in a model system by demonstrating an improvement of seed yield (e.g. in terms of increased seed/grain size, increased ear number, increased seed number per ear, improvement of seed filling, improvement of seed composition, embryo and/or endosperm improvements, and the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under non-stressed conditions), improvement in plant architecture.

Increased yield-related phenotypes may also be measured to determine tolerance to abiotic environmental stress. Abiotic stresses include drought, low temperature, salinity, osmotic stress, shade, high plant density, mechanical stresses, and oxidative stress, and yield-related pheno-types are encompassed by tolerance to such abiotic stresses. Additional phenotypes that can be monitored to determine enhanced tolerance to abiotic environmental stress include, without limitation, wilting; leaf browning; loss of turgor, which results in drooping of leaves or needles stems, and flowers; drooping and/or shedding of leaves or needles; the leaves are green but leaf angled slightly toward the ground compared with controls; leaf blades begun to fold (curl) inward; premature senescence of leaves or needles; loss of chlorophyll in leaves or needles and/or yellowing. Any of the yield-related phenotypes described above may be monitored in field tests or in model plants to demonstrate that a transgenic plant has increased tolerance to abiotic environmental stress.

A polypeptide conferring an yield-increasing activity can be encoded by a respective nucleic acid sequence as shown in Table I, column 5 or 8, and/or comprises or consists of a respective polypeptide as depicted in Table II, column 5 and 8, and/or can be amplified with the respective primer set shown in Table III, column 8, in case in column 7 oleic acid, 2-hydroxybehenic acid, 2-hydroxytetracosenic acid (preferably 2-hydroxynervonic acid), behenic acid, cerebronic acid or nervonic acid is indicated.

“Improved adaptation” to environmental stress like e.g. freezing and/or chilling temperatures refers to an improved plant performance under environmental stress conditions.

A modification, i.e. an increase, can be caused by endogenous or exogenous factors. For example, an increase in activity in an organism or a part thereof can be caused by adding a gene product or a precursor or an activator or an agonist to the media or nutrition or can be caused by introducing said subjects into an organism, transient or stable. Furthermore such an increase can be reached by the introduction of the respective inventive nucleic acid sequence or the encoded protein in the correct cell compartment for example into the nucleus or cytoplasmic respectively or into plastids either by transformation and/or targeting.

The term “yield” as used herein generally refers to a measurable produce from a plant, particularly a crop. Yield and yield increase (in comparison to a non-transformed starting or wild-type plant) can be measured in a number of ways, and it is understood that a skilled person will be able to apply the correct meaning in view of the particular embodiments, the particular crop concerned and the specific purpose or application concerned. The terms “improved yield” or “increased yield” can be used interchangeable.

As used herein, the term “improved yield” or the term “increased yield” means any improvement in the yield of any measured plant product, such as grain, fruit or fiber. In accordance with the invention, changes in different phenotypic traits may improve yield. For example, and without limitation, parameters such as floral organ development, root initiation, root biomass, seed number, seed weight, harvest index, tolerance to abiotic environmental stress, leaf formation, phototropism, apical dominance, and fruit development, are suitable measurements of improved yield. Any increase in yield is an improved yield in accordance with the invention. For example, the improvement in yield can comprise a 0.1%, 0.5%, 1%, 3%, 5%, 10%, 15%, 20%, 30%, 40%, 50%, 60%, 70%, 80%, 90% or greater increase in any measured parameter. For example, an increase in the bushel/acre yield of soybeans or corn derived from a crop comprising plants which are transgenic for the nucleotides and polypeptides of Table I, in case in column 7 oleic acid, 2-hydroxybehenic acid, 2-hydroxytetracosenic acid (preferably 2-hydroxynervonic acid), behenic acid, cerebronic acid or nervonic acid is mentioned, as compared with the bushel/acre yield from untreated soybeans or corn cultivated under the same conditions, is an improved yield in accordance with the invention. The increased or improved yield can be achieved in the absence or presence of stress conditions.

For example, enhanced or increased “yield” refers to one or more yield parameters selected from the group consisting of biomass yield, dry biomass yield, aerial dry biomass yield, underground dry biomass yield, fresh-weight biomass yield, aerial fresh-weight biomass yield, underground fresh-weight biomass yield; enhanced yield of harvestable parts, either dry or fresh-weight or both, either aerial or underground or both; enhanced yield of crop fruit, either dry or fresh-weight or both, either aerial or underground or both; and preferably enhanced yield of seeds, either dry or fresh-weight or both, either aerial or underground or both.

Crop yield is defined herein as the number of bushels of relevant agricultural product (such as grain, forage, or seed) harvested per acre. Crop yield is impacted by abiotic stresses, such as drought, heat, salinity, and cold stress, and by the size (biomass) of the plant. Traditional plant breeding strategies are relatively slow and have in general not been successful in conferring increased tolerance to abiotic stresses. Grain yield improvements by conventional breeding have nearly reached a plateau in maize.

Accordingly, the yield of a plant can depend on the specific plant/crop of interest as well as its intended application (such as food production, feed production, processed food production, biofuel, biogas or alcohol production, or the like) of interest in each particular case. Thus, in one embodiment, yield is calculated as harvest index (expressed as a ratio of the weight of the respective harvestable parts divided by the total biomass), harvestable parts weight per area (acre, square meter, or the like); and the like. The harvest index, i.e., the ratio of yield biomass to the total cumulative biomass at harvest, in maize has remained essentially unchanged during selective breeding for grain yield over the last hundred years. Accordingly, recent yield improvements that have occurred in maize are the result of the increased total biomass production per unit land area. This increased total biomass has been achieved by increasing planting density, which has led to adaptive phenotypic alterations, such as a reduction in leaf angle, which may reduce shading of lower leaves, and tassel size, which may increase harvest index. Harvest index is relatively stable under many environmental conditions, and so a robust correlation between plant size and grain yield is possible. Plant size and grain yield are intrinsically linked, because the majority of grain biomass is dependent on current or stored photosynthetic productivity by the leaves and stem of the plant. As with abiotic stress tolerance, measurements of plant size in early development, under standardized conditions in a growth chamber or greenhouse, are standard practices to measure potential yield advantages conferred by the presence of a transgene.

Acccordingly, the yield of a plant can be increased by improving one or more of the yield-related phenotypes.

Such yield-related phenotypes or traits of a plant the improvement of which results in increased yield comprise, without limitation, the increase of the intrinsic yield capacity of a plant and/or increased stress tolerance, e.g. improved nutrient use efficiency, improved drought tolerance

For example, yield refers to biomass yield, e.g. to dry weight biomass yield and/or fresh-weight biomass yield. Biomass yield refers to the aerial or underground parts of a plant, depending on the specific circumstances (test conditions, specific crop of interest, application of interest, and the like). In one embodiment, biomass yield refers to the aerial and underground parts. Biomass yield may be calculated as fresh-weight, dry weight or a moisture adjusted basis. Biomass yield may be calculated on a per plant basis or in relation to a specific area (e.g. biomass yield per acre/ square meter/ or the like).

In other embodiment, “yield” refers to seed yield which can be measured by one or more of the following parameters: number of seeds or number of filled seeds (per plant or per area (acre/square meter/ or the like)); seed filling rate (ratio between number of filled seeds and total number of seeds); number of flowers per plant; seed biomass or total seeds weight (per plant or per area (acre/square meter/ or the like); thousand kernel weight (TKW; extrapolated from the number of filled seeds counted and their total weight; an increase in TKW may be caused by an increased seed size, an increased seed weight, an increased embryo size, and/or an increased endosperm). Other parameters allowing to measure seed yield are also known in the art. Seed yield may be determined on a dry weight or on a fresh weight basis, or typically on a moisture adjusted basis, e.g. at 15.5 percent moisture.

For example, the term “increased yield” means that the a plant, exhibits an increased growth rate, under conditions of abiotic environmental stress, compared to the corresponding wild-type plant.

An increased growth rate may be reflected inter alia by or confers an increased biomass production of the whole plant, or an increased biomass production of the aerial parts of a plant, or by an increased biomass production of the underground parts of a plant, or by an increased biomass production of parts of a plant, like stems, leaves, blossoms, fruits, and/or seeds.

Increased yield includes higher fruit yields, higher seed yields, higher fresh matter production, and/or higher dry matter production.

The term “increased yield” means that the plant, exhibits a prolonged growth under conditions of abiotic environmental stress, as compared to the corresponding, e.g. non-transformed, wild type organism. A prolonged growth comprises survival and/or continued growth of the plant, at the moment when the non-transformed wild type organism shows visual symptoms of deficiency and/or death.

When the plant of the invention is a corn plant increased yield for corn plants means, for example, increased seed yield, in particular for corn varieties used for feed or food. Increased seed yield of corn refers to an increased kernel size or weight, an increased kernel per ear, or increased ears per plant. Alternatively or in addition the cob yield may be increased, or the length or size of the cob is increased, or the kernel per cob ratio is improved.

When the plant of the invention is a soy plant, increased yield for soy plants means increased seed yield, in particular for soy varieties used for feed or food. Increased seed yield of soy refers for example to an increased kernel size or weight, an increased kernel per pod, or increased pods per plant.

When the plant of the invention is an oil seed rape (OSR) plant, increased yield for OSR plants means increased seed yield, in particular for OSR varieties used for feed or food. Increased seed yield of OSR refers to an increased seed size or weight, an increased seed number per silique, or increased siliques per plant.

When the plant of the invention is a cotton plant, increased yield for cotton plants means increased lint yield. Increased lint yield of cotton refers in one embodiment to an increased length of lint.

Said increased yield can typically be achieved by enhancing or improving, one or more yieldrelated traits of the plant. Such yield-related traits of a plant comprise, without limitation, the increase of the intrinsic yield capacity of a plant, improved nutrient use efficiency, and/or increased stress tolerance, like improved nutrient use efficiency, in particular increased abiotic stress tolerance, e.g. improved nutrient use efficiency and/or improved water use efficiency.

Intrinsic yield capacity of a plant can be, for example, manifested by improving the specific (intrinsic) seed yield (e.g. in terms of increased seed/ grain size, increased ear number, increased seed number per ear, improvement of seed filling, improvement of seed composition, embryo and/or endosperm improvements, or the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under stressed or non-stressed conditions), improvement in plant architecture, cell cycle modifications, photosynthesis modifications, various signaling pathway modifications, modification of transcriptional regulation, modification of translational regulation, modification of enzyme activities, and the like); and/or the like.

The improvement or increase of stress tolerance of a plant can for example be manifested by improving or increasing a plant's tolerance against stress, particularly abiotic stress. In the present application, abiotic stress refers generally to abiotic environmental conditions a plant is typically confronted with, including, but not limited to, drought (tolerance to drought may be achieved as a result of improved water use efficiency), heat, low temperatures and cold conditions (such as freezing and chilling conditions), nutrient depetion, salinity, osmotic stress, shade, high plant density, mechanical stress, oxidative stress, and the like.

The increased plant yield can for example be mediated by increasing the “nutrient use efficiency of a plant”, e.g. by improving the use efficiency of nutrients including, but not limited to, phosphorus, potassium, and nitrogen. Further, higher yields may be obtained with current or standard levels of nitrogen use.

Generally, the term “increased tolerance to stress” can be defined as survival of plants, and/or higher yield production, under stress conditions as compared to a non-transformed wild type or starting plant: For example, the plant of the invention or produced according to the method of the invention is better adapted to the stress conditions. “Improved adaptation” to environmental stress like e.g. drought, heat, nutrient depletion, freezing and/or chilling temperatures refers herein to an improved plant performance resulting in an increased yield, particularly with regard to one or more of the yield related traits as defined in more detail above.

During its life-cycle, a plant is generally confronted with a diversity of environmental conditions. Any such conditions, which may, under certain circumstances, have an impact on plant yield, are herein referred to as “stress” condition. Environmental stresses may generally be divided into biotic and abiotic (environmental) stresses. Unfavorable nutrient conditions are also referred to as “environmental stress”. The present invention does also contemplate solutions for this kind of environmental stress, e.g. referring to increased nutrient use efficiency.

For the purposes of the description of the present invention, the terms “enhanced tolerance to abiotic stress”, “enhanced resistance to abiotic environmental stress”, “enhanced tolerance to environmental stress”, “improved adaptation to environmental stress” and other variations and expressions similar in its meaning are used interchangeably and refer, without limitation, to an improvement in tolerance to one or more abiotic environmental stress(es) as described herein and as compared to a corresponding origin or wild type plant or a part thereof.

The term abiotic stress tolerance(s) refers for example low temperature tolerance, drought tolerance or improved water use efficiency (WUE), heat tolerance, salt stress tolerance, improved nutrient efficiency, like nitrogen use efficiency and others. Studies of a plant's response to desiccation, osmotic shock, and temperature extremes are also employed to determine the plant's tolerance or resistance to abiotic stresses. Water use efficiency (WUE) is a parameter often correlated with drought tolerance. In selecting traits for improving crops, a decrease in water use, without a change in growth would have particular merit in an irrigated agricultural system where the water input costs were high. An increase in growth without a corresponding jump in water use would have applicability to all agricultural systems. In many agricultural systems where water supply is not limiting, an increase in growth, even if it came at the expense of an increase in water use also increases yield. Drought stress means any environmental stress which leads to a lack of water in plants or reduction of water supply to plants, including a secondary stress by low temperature and/or salt, and/or a primary stress during drought or heat, e.g. desiccation etc.

### Specific Embodiments

Accordingly, this invention provides respective measures and methods to produce plants with increased yield, e.g. genes conferring an increased yield-related trait, for example enhanced tolerance to abiotic environmental stress, for example an increased drought tolerance and/or low temperature tolerance and/or an increased nutrient use efficiency, intrinsic yield and/or another increased yield-related trait, upon expression or over-expression, especially increased intrinsic yield. Accordingly, the present invention provides such genes in case in column 7 of the respective Table “oleic acid” is indicated. In particular, such genes are described in column 5 as well as in column 8 of Tables I, especially the coding region thereof, or homologs or fragments thereof, in case oleic acid is indicated in column 7 or the respective polypeptides are described in column 5 as well as in column 8 of Table II, or homologs or fragments thereof, in case oleic acid is indicated in column 7.

Accordingly, the present invention provides respective transgenic plants showing one or more improved yield-related traits, especially enhanced intrinsic yield capacity as compared to the corresponding origin or the wild type plant and methods for producing such transgenic plants with increased yield in case in column 7 of the respective Table “oleic” is indicated.

In one embodiment, one or more of said yield-increasing activities are increased by increasing the amount and/or the specific activity of one or more proteins listed in Table I, column 5 or 8 in a compartment of a cell indicated in Table I, column 6, in case in column 7 of the respective Table “oleic acid” is indicated.

Accordingly to present invention, the yield of the plant of the invention is increased by improving one or more of the yield-related traits as defined herein. Said increased yield in accordance with the present invention can typically be achieved by enhancing or improving, in comparison to an origin or wild-type plant, one or more yield-related traits of said plant. Such yield-related traits of a plant the improvement of which results in increased yield comprise, without limitation, the increase of the intrinsic yield capacity of a plant, and/or increased stress tolerance, e.g. improved nutrient use efficiency, like nitrogen use efficiency,.

Accordingly, this invention provides respective measures and methods to produce plants with increased yield, e.g. genes conferring an increased yield-related trait, for example enhanced tolerance to abiotic environmental stress, for example an increased drought tolerance and/or low temperature tolerance and/or an increased nutrient use efficiency, intrinsic yield and/or another increased yield-related trait, upon expression or over-expression, especially enhanced tolerance to abiotic stress, in particular increased water use efficiency/drought tolerance. Accordingly, the present invention provides such genes in case in column 7 of the respective Table “2-hydroxybehenic acid, 2-hydroxytetracosenic acid (preferably 2-hydroxynervonic acid), behenic acid, cerebronic acid or nervonic acid” is indicated. In particular, such genes are described in column 5 as well as in column 8 of Tables I, especially the coding region thereof, or homologs or fragments thereof, in case 2-hydroxybehenic acid, 2-hydroxytetracosenic acid (preferably 2-hydroxynervonic acid), behenic acid, cerebronic acid or nervonic acid is indicated in column 7 or the respective polypeptides are described in column 5 as well as in column 8 of Table II, or homologs or fragments thereof, in case 2-hydroxybehenic acid, 2-hydroxytetracosenic acid (preferably 2-hydroxynervonic acid), behenic acid, cerebronic acid or nervonic acid is indicated in column 7.

Accordingly, the present invention provides respective transgenic plants showing one or more improved yield-related traits, especially improved water use efficiency/drought tolerance ,as compared to the corresponding origin or the wild type plant and methods for producing such transgenic plants with increased yield in case in column 7 of the respective Table “2-hydroxybehenic acid, 2-hydroxytetracosenic acid (preferably 2-hydroxynervonic acid), behenic acid, cerebronic acid or nervonic acid” is indicated.

In one embodiment, one or more of said yield-increasing activities are increased by increasing the amount and/or the specific activity of one or more proteins listed in Table I, column 5 or 8 in a compartment of a cell indicated in Table I, column 6, in case in column 7 of the respective Table “2-hydroxybehenic acid, 2-hydroxytetracosenic acid (preferably 2-hydroxynervonic acid), behenic acid, cerebronic acid or nervonic acid” is indicated.

Accordingly to present invention, the yield of the plant of the invention is increased by improving one or more of the yield-related traits as defined herein. Said increased yield in accordance with the present invention can typically be achieved by enhancing or improving, in comparison to an origin or wild-type plant, one or more yield-related traits of said plant. Such yield-related traits of a plant the improvement of which results in increased yield comprise, without limitation, the increase of the intrinsic yield capacity of a plant, improved nutrient use efficiency, like nitrogen use efficiency, and/or increased stress tolerance, e.g. improved nutrient use efficiency, like nitrogen use efficiency, or improved water use efficiency.

In a further embodiment the present invention is characterized by the features as disclosed in the following items:

Item 1. A process for the production of a fine chemical selected from the group consisting of: 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid and oleic acid
(a) increasing or generating one or more activities selected from the group consisting of 2,3-dihydroxyphenylpropionate 1,2-dioxygenase, 2-C-methyl-D-erythritol 4-phosphate cytidylyltransferase, 30S ribosomal protein S3, 3-ketoacyl-CoA thiolase, 3-oxoacyl-[acyl-carrier-protein] reductase, 3-oxoacyl-[acyl-carrier-protein] synthase, 40S ribosomal protein S17, ABC transporter component, ABC transporter permease protein, acetyl-coenzyme A synthetase, acid shock protein, acyl-CoA synthase, acyl-desaturase, adenosine kinase, aldolase, alkaline phosphatase isozyme conversion protein, amino acid ABC transporter permease protein, aminotransferase, ankyrin repeat protein, At2g33510-protein, ATPase epsilon subunit, ATP-binding component of a transport system, b0050-protein, b0249-protein, b0371-protein, b0518-protein, b0801-protein, b1046-protein, b1346-protein, b1470-protein, b1522-protein, b1670-protein, b1703-protein, b1837-protein, b2032-protein, b2099-protein, b2107-protein, b2126-protein, b2139-protein, b2360-protein, b2474-protein, b2513-protein, b2654-protein, b3442-protein, b3644-protein, b3812-protein, b3944-protein, b4023-protein, b4205-protein, beta-hydroxylase, betaine aldehyde dehydrogenase, bifunctional protein (phosphoribosyltransferase and regulatory protein), bifunctional purine biosynthesis protein, binding-protein-dependent transport systems inner membrane component, biotin synthase, calcium-dependent protein kinase, carbon dioxide concentrating mechanism protein, cardiolipin synthase, CDP-diacylglycerol-serine O-phosphatidyltransferase, cell division control protein, cell wall protein, chlorophyllase, citrate synthase, coproporphyrinogen III oxidase, cyclin, cystathionine gamma-synthase, cysteine synthase A, dihydrolipoamide dehydrogenase, DNA mismatch repair protein, DnaJ-like chaperone, electron transport complex protein, Elongation factor Tu, ethanolamine utilization protein, F-box protein, ferredoxin, fructose 1,6-bisphosphatase, fumarylacetoacetate hydrolase, glutamate 5-kinase, glutamate-ammonia-ligase, glutaredoxin, glycerol-3-phosphate dehydrogenase, glycine cleavage system H protein, GM02LC38418-protein, heat shock protein, heat shock transcription factor, H esB/Yad R/YfhF family protein, homocitrate synthase, hydrolase, isocitrate dehydrogenase, lipoprotein precursor, malate dehydrogenase, malonyl CoA-acyl carrier protein transacylase, metal-dependent hydrolase, molecular chaperone, molecular chaperone portein, monothiol glutaredoxin, multiple antibiotic resistance protein, murein transglycosylase, N-acetyl-gamma-glutamyl-phosphate reductase, NADP-dependent malic enzyme, outer-membrane lipoprotein precursor, oxidoreductase, permease protein of ABC transporter, phage-related repressor protein, phosphatase, phosphate import ATP-binding protein, phosphatidylserine synthase, phospho-2-dehydro-3-deoxyheptonate aldolase, phosphoadenosine phosphosulfate reductase, phosphotransferase, phosphotransferase system component, polygalacturonase, protein kinase, protein phosphatase, purine nucleoside phosphorylase, pyridoxamine kinase, recombinase A, sec-independent protein translocase, Sec-independent protein translocase subunit, sensor histidine kinase, sensor kinase, sll0281-protein, sll0406-protein, small nuclear ribonucleoprotein , sodium/proton antiporter, squalene monooxygenase, sugar transport system permease protein, threonine dehydratase, transcription factor, transcriptional regulator, transcriptional regulator protein, transport protein, tryptophan synthase alpha chain, TTC1386-protein, vacuolar processing enzyme, ycl027w-protein, ycl049c-protein, ydr273w-protein, ydr339c-protein, YFL019C-protein, ygl237c-protein, YGR104C-protein, yhr033w-protein, YJL127W-A-protein, ylr326w-protein, yml083c-protein, yml084w-protein, and ynl321w-protein, in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid or oleic acid, respectively,or a composition comprising 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid or oleic acid, respectively, in said non-human organism or in the culture medium surrounding said non-human organism.

Item 2. A process for the production of a respective fine chemical selected from the group consisting of 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid and oleic acid, which comprises
(A)
   (i) increasing or generating of the expression of; and/or
   (ii) increasing or generating the expression of an expression product of; and/or
   (iii) increasing or generating one or more activities of an expression product encoded by; at least one respective nucleic acid molecule comprising a nucleic acid molecule selected from the group consisting of:
   (a) a nucleic acid molecule encoding the polypeptide shown in column 5 or 8 of Table II, preferably Table II B, application no.12, or a homolog or a fragment thereof, whereby the respective line discloses in column 7 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid or oleic acid, respectively;
   (b) a nucleic acid molecule shown in column 5 or 8 of Table I, preferably Table I B, application no.12, or a homolog or a fragment thereof (preferably the coding region thereof), whereby the respective line discloses in column 7 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid or oleic acid, respectively;
   (c) a nucleic acid molecule, which, as a result of the degeneracy of the genetic code, can be derived from a polypeptide sequence depicted in column 5 or 8 of Table II, preferably Table II B, application no.12, whereby the respective line discloses in column 7 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid or oleic acid, respectively;
   (d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 12, or the coding region thereof, whereby the respective line discloses in column 7 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid or oleic acid, respectively;
   (e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no.12, whereby the respective line discloses in column 7 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid or oleic acid, respectively;
   (i) a nucleic acid molecule encoding a polypeptide having the activity represented by a respective protein as depicted in column 5 of Table II, application no. 12, whereby the respective line discloses in column 7 2-hydroxybehenic acid, 2-hydroxypalmitic acid, 2-hydroxytetracosenoic acid (preferably 2-hydroxy-nervonic acid), behenic acid, cerebronic acid, eicosanoic acid, heptadecanoic acid, hexadecadienoic acid, hexadecatrienoic acid, lauric acid, myristic acid, nervonic acid or oleic acid, respectively;
   (j) a nucleic acid molecule which comprises a polynucleotide, which is obtained by amplifying a cDNA library or a genomic library using the primers as depicted in column 8 of Table III, application no. 12, whereby the respective line discloses in column 7 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid or oleic acid, respectively; and
   (k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
   or a nucleic acid molecule comprising a sequence which is complementary thereto;
   in a non-human organism or a part thereof, preferably a microorganism, a plant cell, a plant or a part thereof, as compared to a corresponding non-transformed wild type non-human organism or a part thereof; and
(B) growing the non-human organism or a part thereof under conditions which permit the production of 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid or oleic acid, respectively, or a composition comprising 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid or oleic acid, respectively, in said non-human organism or in the culture medium surrounding said non-human organism.

Item 3. A process of items 1 or 2, comprising of recovering the respective fine chemical 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid, or oleic acid in its free or bound form.

Item 4. The process of any one of items 1 to 3, comprising the following steps:
(a) selecting a non-human organism or a part thereof expressing a polypeptide encoded by the nucleic acid molecule characterized in item 2;
(b) mutagenizing the selected non-human organism or the part thereof;
(c) comparing the activity or the expression level of said polypeptide in the mutagenized non-human organism or the part thereof with the activity or the expression of said polypeptide of the selected non-human organisms or the part thereof;
(d) selecting the mutated non-human organisms or parts thereof, which comprise an increased activity or expression level of said polypeptide compared to the selected non-human organism or the part thereof;
(e) optionally, growing and cultivating the non-human organisms or the parts thereof;
and
(f) recovering, and optionally isolating, the free or bound 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid or oleic acid, respectively, produced by the selected mutated non-human organisms or parts thereof.

Item 5. The process of any one of items 1 to 4 wherein the activity of said protein or the expression of said nucleic acid molecule is increased or generated transiently or stably.

Item 6. An isolated nucleic acid molecule comprising a nucleic acid molecule selected from the group consisting of:
(a) a nucleic acid molecule encoding a polypeptide shown in Table II, application no. 12, column 5 or 8, preferably shown in Table II B, application no. 12, column 8, or a homolog or a fragment thereof;
(b) a nucleic acid molecule shown in Table I, application no. 12, column 5 or 8, preferably shown in Table I B, application no. 12, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 12, preferably in column 8 of Table II B, application no. application no.,
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 3, preferably shown in column 8 of Table I B, application no. 12, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 12;
(i) a nucleic acid molecule encoding a polypeptide having the activity represented by a respective protein as depicted in column 5 of Table II, application no. 12;
(j) a nucleic acid molecule comprising a polynucleotide which is obtained by amplifying a cDNA library or a genomic library using the primers in Table III, application no. 12, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
or a nucleic acid molecule comprising a sequence which is complementary thereto.

Item 7. A nucleic acid construct which confers the expression of the nucleic acid molecule as defined in item 6, comprising one or more regulatory elements.

Item 8. An expression cassette comprising
(a) a promoter, preferably selected from the group consisting of Big35S, PCUbi, Super and USP;
(b) a nucleic acid molecule as defined in item 6.

Item 9. A vector comprising the nucleic acid molecule as defined in item 6 or the nucleic acid construct as claimed in item 7 or the expression cassette as claimed in item 8.

Item 10. A host cell or a plant organelle, which has been transformed with the vector as claimed in item 9 or the nucleic acid molecule as defined in item 6 or the nucleic acid construct as claimed in item 7 or the expression cassette as claimed in item 8.

Item 11. The host cell of item 10, which is a plant cell or a microorganism.

Item 12. A process for producing a polypeptide, wherein the polypeptide is expressed in a host cell as claimed in item 10 or 11.

Item 13. A polypeptide produced by the process as claimed in item 12 or encoded by the nucleic acid molecule as defined in item 6.

Item 14. An antibody, which binds specifically to the polypeptide as claimed in item 13.

Item 15. A plant tissue, propagation material, harvested material or a plant or a part thereof comprising the host cell as claimed in item 10 or 11.

Item 16. A process for the identification of a compound conferring an increase in 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid or oleic acid production, respectively, in a non-human organism, comprising the steps:
(a) culturing a plant cell or tissue or microorganism or maintaining a plant expressing the polypeptide encoded by the nucleic acid molecule as defined in item 6 conferring an increase in the amount of 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid or oleic acid, respectively, in a non-human organism or a part thereof and a readout system capable of interacting with the polypeptide under suitable conditions which permit the interaction of the polypeptide with said readout system in the presence of a compound or a sample comprising a plurality of compounds and capable of providing a detectable signal in response to the binding of a compound to said polypeptide under conditions which permit the expression of said readout system and of the polypeptide encoded by the nucleic acid molecule of item 6 conferring an increase in the amount of 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid or oleic acid, respectively, in a non-human organism or a part thereof;
(b) identifying if the compound is an effective agonist by detecting the presence or absence or increase of a signal produced by said readout system.

Item 17. A method for the production of an agricultural composition comprising the steps of the method of item 16 and formulating the compound identified in item 16 in a form acceptable for an application in agriculture.

Item 18. A composition comprising the nucleic acid molecule as defined in item 6, the polypeptide as claimed in item 13, the nucleic acid construct as claimed in item 7, the expression cassette as claimed in item 8, the vector as claimed in item 9, the compound as claimed in item 16, the antibody as claimed in item 14, and optionally an agricultural acceptable carrier.

Item 19. Use of the nucleic acid molecule as defined in item 6 for the identification of a nucleic acid molecule conferring an increase in 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid or oleic acid after expression.

Item 20. Cosmetic, pharmaceutical, food or feed composition comprising the nucleic acid molecule as defined in item 6, the polypeptide as claimed in item 13, the nucleic acid construct as claimed in item 7, the expression cassette as claimed in item 8, the vector as claimed in item 9, the antibody as claimed in item 14, the plant or part thereof, or plant tissue as claimed in item 15, the harvested material or propagation material as claimed in item 15 or the host cell of items 10 or 11.

Item 21. Use of the nuclei acid molecule as defined in item 6, the polypeptide as claimed in item 13, the nucleic acid construct as claimed in item 7, the expression cassette as claimed in item 8, the vector as claimed in claim 9, the plant or plant tissue as claimed in

Item 15, or the host cell of item 10 to 11 for the production of plant resistant to a herbicide inhibiting the production of 2-hydroxybehenic acid, 2-hydroxypalmitic acid, 2-hydroxytetracosenoic acid (preferably 2-hydroxynervonic acid), behenic acid, cerebronic acid, eicosanoic acid, heptadecanoic acid, hexadecadienoic acid, hexadecatrienoic acid, lauric acid, myristic acid, nervonic acid or oleic acid, respectively.

Item 22. A method for producing a plant with increased yield as compared to a corresponding non-transformed wild type plant, whereby the method comprises
(i) increasing or generating of the expression of; and/or
(ii) increasing or generating the expression of an expression product of; and/or
(iii) increasing or generating one or more activities of an expression product encoded by; at least one respective nucleic acid molecule comprising a nucleic acid molecule selected from the group consisting of:
(a) a nucleic acid molecule encoding the polypeptide shown in column 5 or 8 of Table II, preferably Table II B, application no. 12, or a homolog or a fragment thereof, in case in column 7 2-hydroxybehenic acid, 2-hydroxytetracosenoic acid (preferably 2hydroxynervonic acid), behenic acid, cerebronic acid, nervonic acid or oleic acid, respectively, is indicated;
(b) a nucleic acid molecule shown in column 5 or 8 of Table I, preferably Table I B, application no. 12, or a homolog or a fragment thereof (preferably the coding region thereof) , in case in column 7 2-hydroxybehenic acid, 2-hydroxytetracosenoic acid, (preferably 2-hydroxynervonic acid), behenic acid, cerebronic acid, nervonic acid or oleic acid, respectively, is indicated;
(c) a nucleic acid molecule, which, as a result of the degeneracy of the genetic code, can be derived from a polypeptide sequence depicted in column 5 or 8 of Table II, preferably Table II B, application no. 12, in case in column 7 2-hydroxybehenic acid, 2-hydroxytetracosenoic acid (preferably 2-hydroxynervonic acid), behenic acid, cerebronic acid, nervonic acid or oleic acid, respectively, is indicated;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 12, or the coding region thereof, in case in column 7 2-hydroxybehenic acid, 2-hydroxytetracosenoic acid (preferably 2-hydroxynervonic acid), behenic acid, cerebronic acid, nervonic acid or oleic acid, respectively, is indicated;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 12, in case in column 7 2-hydroxybehenic acid, 2-hydroxytetracosenoic acid (preferably 2-hydroxynervonic acid), behenic acid, cerebronic acid, nervonic acid or oleic acid, respectively, is indicated;
(i) a nucleic acid molecule encoding a polypeptide having the activity represented by a respective protein as depicted in column 5 of Table II, application no. 12, in case in column 7 2-hydroxybehenic acid, 2-hydroxytetracosenoic acid (preferably 2hydroxynervonic acid), behenic acid, cerebronic acid, nervonic acid or oleic acid, respectively, is indicated;
(j) a nucleic acid molecule which comprises a polynucleotide, which is obtained by amplifying a cDNA library or a genomic library using the primers as depicted in column 8 of Table III, application no. 12, in case in column 7 2-hydroxybehenic acid, 2-hydroxytetracosenoic acid (preferably 2-hydroxynervonic acid), behenic acid, cerebronic acid, nervonic acid or oleic acid, respectively, is indicated; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (k);
or a nucleic acid molecule comprising a sequence which is complementary thereto; in a plant cell, plant or a part thereof.

Item 23. A method for producing a plant with increased yield according to item 22, which comprises in addition the regeneration of a plant from such transformed plant cell, plant cell compartment, plant cell nucleus, plant cell or plant tissue with increased yield.

[0483.1.8.12] to [0494.1.8.12] for the disclosure of these paragraphs see [0483.1.8.8] to [0494.1.8.8] above

### Example 11a

### PCR Amplification of the Sequences

Unless otherwise specified, standard methods as described in Sambrook et al., Molecular Cloning: A laboratory manual, Cold Spring Harbor 1989, Cold Spring Harbor Laboratory Press are used.

The inventive sequences as shown in the respective line in Table I, column 5, preferably the coding region thereof, were amplified by PCR as described in the protocol of the Pfu Ultra, Pfu Turbo or Herculase DNA polymerase (Stratagene). The composition for the protocol of the Pfu Ultra, Pfu Turbo or Herculase DNA polymerase was as follows: 1×PCR buffer (Stratagene), 0.2 mM of each dNTP, 100 ng genomic DNA of Saccharomyces cerevisiae (strain S288C; Research Genetics, Inc., now Invitrogen), Escherichia coli (strain MG1655; E. coli Genetic Stock Center), Synechocystis sp. (strain PCC6803), Azotobacter vinelandii (strain N.R. Smith,16), Thermus thermophilus (HB8) or 50 ng cDNA from various tissues and development stages of Arabidopsis thaliana (ecotype Columbia), Physcomitrella patens, Glycine max (variety Resnick), Brassica napus, Oryza sativa or Zea mays (variety B73, Mo17, A188), 50 pmol forward primer, 50 pmol reverse primer, with or without 1 M Betaine, 2.5 u Pfu Ultra, Pfu Turbo or Herculase DNA polymerase.

The amplification cycles were as follows:

1 cycle of 2-3 minutes at 94-95° C., then 25-36 cycles with 30-60 seconds at 94-95° C., 30-45 seconds at 50-60° C. and 210-480 seconds at 72° C., followed by 1 cycle of 5-10 minutes at 72° C., then 4-16° C.—preferably for Saccharomyces cerevisiae, Escherichia coli, Synechocystis sp., Azotobacter vinelandii, Thermus thermophilus.

In case of Arabidopsis thaliana, Brassica napus, Glycine max, Oryza sativa, Physcomitrella patens, Zea mays the amplification cycles were as follows:

1 cycle with 30 seconds at 94° C., 30 seconds at 61° C., 15 minutes at 72° C., then 2 cycles with 30 seconds at 94° C., 30 seconds at 60° C., 15 minutes at 72° C., then 3 cycles with 30 seconds at 94° C., 30 seconds at 59° C., 15 minutes at 72° C., then 4 cycles with 30 seconds at 94° C., 30 seconds at 58° C., 15 minutes at 72° C., then 25 cycles with 30 seconds at 94° C., 30 seconds at 57° C., 15 minutes at 72° C., then 1 cycle with 10 minutes at 72° C., then finally 4-16° C.

RNAs were generated with the RNeasy Plant Kit according to the standard protocol (Qiagen) and Superscript II Reverse Transkriptase was used to produce double stranded cDNA according to the standard protocol (Invitrogen).

ORF specific primer pairs for the genes to be expressed are shown in the respective line in Table III, column 8. The following adapter sequences were added to Saccharomyces cerevisiae ORF specific primers (see Table III) for cloning purposes:

SEQ ID NO: 20

foward primer: 5′-GGAATTCCAGCTGACCACC-3′ i)

SEQ ID NO: 21

reverse primer: 5′-GATCCCCGGGAATTGCCATG-3′ ii)

These adaptor sequences allow cloning of the ORF into the various vectors containing the Resgen adaptors, see table column E of Table c.

The following adapter sequences were added to Saccharomyces cerevisiae, Escherichia coli, Synechocystis sp., Azotobacter vinelandii, Thermus thermophilus, Arabidopsis thaliana, Brassica napus, Glycine max, Oryza sativa , Physcomitrella patens, or Zea mays ORF specific primers for cloning purposes:

SEQ ID NO: 22

forward primer: 5′-TTGCTCTTCC- 3′ iii)

SEQ ID NO: 23

reverse primer: 5′-TTGCTCTTCG-3″ iiii)

The adaptor sequences allow cloning of the ORF into the various vectors containing the Colic adaptors, see table column E of Table c.

Therefore for amplification and cloning of Saccharomyces cerevisiae SEQ ID NO: 14843, a primer consisting of the adaptor sequence i) and the ORF specific sequence SEQ ID NO: 14871 and a second primer consisting of the adaptor sequence ii) and the ORF specific sequence SEQ ID NO: 14872 were used.

For amplification and cloning of Escherichia coli SEQ ID NO: 7269, a primer consisting of the adaptor sequence iii) and the ORF specific sequence SEQ ID NO: 7323 and a second primer consisting of the adaptor sequence iiii) and the ORF specific sequence SEQ ID NO: 7324 were used.

For amplification and cloning of Synechocystis sp. SEQ ID NO: 11471, a primer consisting of the adaptor sequence iii) and the ORF specific sequence SEQ ID NO: 11983 and a second primer consisting of the adaptor sequence iiii) and the ORF specific sequence SEQ ID NO: 11984 were used.

For amplification and cloning of Arabidopsis thaliana SEQ ID NO: 1061, a primer consisting of the adaptor sequence iii) and the ORF specific sequence SEQ ID NO: 1293 and a second primer consisting of the adaptor sequence iiii) and the ORF specific sequence SEQ ID NO: 1294 were used.

For amplification and cloning of Azotobacter vinelandii SEQ ID NO: 6040, a primer consisting of the adaptor sequence iii) and the ORF specific sequence SEQ ID NO: 6070 and a second primer consisting of the adaptor sequence iiii) and the ORF specific sequence SEQ ID NO: 6071 were used.

For amplification and cloning of Glycine max SEQ ID NO: 10811, a primer consisting of the adaptor sequence iii) and the ORF specific sequence SEQ ID NO: 11207 and a second primer consisting of the adaptor sequence iiii) and the ORF specific sequence SEQ ID NO: 11208 were used.

For amplification and cloning of Physcomytrella patens SEQ ID NO: 126991, a primer consisting of the adaptor sequence iii) and the ORF specific sequence SEQ ID NO: 127097 and a second primer consisting of the adaptor sequence iiii) and the ORF specific sequence SEQ ID NO: 127098 were used.

For amplification and cloning of Thermus thermophilus SEQ ID NO: 12698, a primer consisting of the adaptor sequence iii) and the ORF specific sequence SEQ ID NO: 12970 and a second primer consisting of the adaptor sequence iiii) and the ORF specific sequence SEQ ID NO: 12971 were used.

Following these examples every sequence disclosed in Table I, preferably column 5, especially the coding region thereof can be cloned by fusing the adaptor sequences to the respective specific primers sequences as disclosed in Table III, column 8 using the respective vectors shown in Table c.

[0496.1.8.12] to [0499.1.8.12] for the disclosure of these paragraphs see [0496.1.8.8] to [0499.1.8.8] above.

### Example 11e

### Cloning of Inventive Sequences as shown in Table I, Column 5 in the Different Expression Vectors

For cloning of for example SEQ ID NO: 7269 from Escherichia coli or any other ORF from Escherichia coli, Synechocystis sp., Azotobacter vinelandii, Thermus thermophilus, Arabidopsis thaliana, Glycine max, or Physcomitrella patens the vector DNA was treated with the restriction enzymes Pacl and Ncol following the standard protocol (MBI Fermentas).

For cloning for example the ORFs of SEQ ID NO: 14843 from Saccharomyces cerevisiae or any other ORF from Saccharomyces cerevisiae into vectors containing the Resgen adaptor sequence the respective vector DNA was treated with the restriction enzyme Ncol.

For cloning of for example ORFs SEQ ID NO: 64218 from Saccharomyces cerevisiae or any other ORF from Saccharomyces cerevisiae into vectors containing the Colic adaptor sequence, the respective vector DNA was treated with the restriction enzymes Pacl and Ncol following the standard protocol (MBI Fermentas).

In all cases the reaction was stopped by inactivation at 70° C. for 20 minutes and purified over QlAquick or NucleoSpin Extract II columns following the standard protocol (Qiagen or Macherey-Nagel).

Then the PCR-product representing the amplified ORF with the respective adapter sequences and the vector DNA were treated with T4 DNA polymerase according to the standard protocol (MBI Fermentas) to produce single stranded overhangs with the parameters 1 unit T4 DNA polymerase at 37° C. for 2-10 minutes for the vector and 1-2 u T4 DNA polymerase at 15-17° C. for 10-60 minutes for the PCR product representing SEQ ID NO: 7269.

The reaction was stopped by addition of high-salt buffer and purified over QlAquick or NucleoSpin Extract II columns following the standard protocol (Qiagen or Macherey-Nagel).

According to this example the skilled person is able to clone all sequences disclosed in Table I, preferably column 5 or column 8, especially the coding region thereof.

Approximately 30-60 ng of prepared vector and a defined amount of prepared amplificate were mixed and hybridized at 65° C. for 15 minutes followed by 37° C. 0.1° C/1 seconds, followed by 37° C. 10 minutes, followed by 0.1° C/1 seconds, then 4-10° C.

The ligated constructs were transformed in the same reaction vessel by addition of competent E. coli cells (strain DHSalpha) and incubation for 20 minutes at 1° C. followed by a heat shock for 90 seconds at 42° C. and cooling to 1-4° C. Then, complete medium (SOC) was added and the mixture was incubated for 45 minutes at 37° C. The entire mixture was subsequently plated onto an agar plate with 0.05 mg/ml kanamycin and incubated overnight at 37° C.

The outcome of the cloning step was verified by amplification with the aid of primers which bind upstream and downstream of the integration site, thus allowing the amplification of the insertion. The amplifications were carried out as described in the protocol of Taq DNA polymerase (Gibco-BRL).

The amplification cycles were as follows:

1 cycle of 1-5 minutes at 94° C., followed by 35 cycles of in each case 15-60 seconds at 94° C., 15-60 seconds at 50-66° C. and 5-15 minutes at 72° C., followed by 1 cycle of 10 minutes at 72° C., then 4-16° C.

Several colonies were checked, but only one colony for which a PCR product of the expected size was detected was used in the following steps.

A portion of this positive colony was transferred into a reaction vessel filled with complete medium (LB) supplemented with kanamycin and incubated overnight at 37° C.

The plasmid preparation was carried out as specified in the Qiaprep or NucleoSpin Multi-96 Plus standard protocol (Qiagen or Macherey-Nagel).

[0501.1.8.12] to [0503.1.8.12] for the disclosure of these paragraphs see [0501.1.8.8] to [0503.1.8.8] above.

Table d showing results of plant analyses

TABLE d

| SeqID | Target | Locus | Metabolite | Source | Promotor | Method | Min | Max |
|---|---|---|---|---|---|---|---|---|
| 113651 | non- | At1g19670 | cerebronic | ARA_LEAF | p-PcUBI | GC | 25 | 38 |
| | targeted | | acid | | | | | |
| 113651 | non- | At1g19670 | 2-hydroxy- | ARA_LEAF | p-PcUBI | GC | 29 | 47 |
| | targeted | | tetracosenoic | | | | | |
| | | | acid (preferably | | | | | |
| | | | 2-hydroxy- | | | | | |
| | | | nervonic acid) | | | | | |
| 1061 | non- | At1g68320 | hexadecatrienoic | ARA_LEAF | p-PcUBI | GC | 13 | 69 |
| | targeted | | acid | | | | | |
| 19419 | non- | At1g72770 | cerebronic | ARA_LEAF | p-PcUBI | GC | 32 | 62 |
| | targeted | | acid | | | | | |
| 19419 | non- | At1g72770 | hexadecatrienoic | ARA_LEAF | p-PcUBI | GC | 14 | 58 |
| | targeted | | acid | | | | | |
| 19419 | non- | At1g72770 | 2-hydroxy- | ARA_LEAF | p-PcUBI | GC | 36 | 74 |
| | targeted | | behenic acid | | | | | |
| 98522 | non- | At2g33510 | 2-hydroxy- | ARA_LEAF | p-PcUBI | GC | 20 | 24 |
| | targeted | | palmitic acid | | | | | |
| 68657 | non- | At3g02990 | 2-hydroxy- | ARA_LEAF | p-PcUBI | GC | 23 | 71 |
| | targeted | | tetracosenoic | | | | | |
| | | | acid | | | | | |
| | | | (preferably 2- | | | | | |
| | | | [symbol] acid) | | | | | |
| 1696 | non- | At3g09820 | cerebronic | ARA_LEAF | p-PcUBI | GC | 26 | 45 |
| | targeted | | acid | | | | | |
| 1696 | non- | At3g09820 | 2-hydroxy- | ARA_LEAF | p-PcUBI | GC | 25 | 52 |
| | targeted | | behenic acid | | | | | |
| 22832 | non- | At3g18524 | 2-hydroxy- | ARA_LEAF | p-PcUBI | GC | 25 | 36 |
| | targeted | | tetracosenoic | | | | | |
| | | | acid | | | | | |
| | | | (preferably 2- | | | | | |
| | | | hydroxy- | | | | | |
| 2935 | non- | At3g62950 | [symbol] cid) | ARA_LEAF | p-PcUBI | GC | 25 | 61 |
| | targeted | | acid | | | | | |
| 2935 | non- | At3g62950 | hexadecatrienoic | ARA_LEAF | p-PcUBI | GC | 17 | 33 |
| | targeted | | acid | | | | | |
| 2935 | non- | At3g62950 | 2-hydroxy- | ARA_LEAF | p-PcUBI | GC | 29 | 57 |
| | targeted | | behenic acid | | | | | |
| 109717 | non- | At4g09960 | 2-hydroxy- | ARA_LEAF | p-PcUBI | GC | 22 | 50 |
| | targeted | | palmitic acid | | | | | |
| 23482 | non- | At4g15660 | 2-hydroxy- | ARA_LEAF | p-PcUBI | GC | 28 | 59 |
| | targeted | | behenic acid | | | | | |
| 23482 | non- | At4g15660 | 2-hydroxy- | ARA_LEAF | p-PcUBI | GC | 23 | 52 |
| | targeted | | palmitic acid | | | | | |
| 3279 | non- | At4g15670 | hexadecatrienoic | ARA_LEAF | p-PcUBI | GC | 26 | 39 |
| | targeted | | acid | | | | | |
| 3279 | non- | At4g15670 | 2-hydroxy- | ARA_LEAF | p-PcUBI | GC | 24 | 104 |
| | targeted | | behenic acid | | | | | |
| 23844 | non- | At4g15690 | cerebronic | ARA_LEAF | p-PcUBI | GC | 23 | 48 |
| | targeted | | acid | | | | | |
| 68849 | non- | At4g26080 | 2-hydroxy- | ARA_LEAF | p-PcUBI | GC | 26 | 48 |
| | targeted | | behenic acid | | | | | |
| 68849 | non- | At4g26080 | 2-hydroxy- | ARA_LEAF | p-PcUBI | GC | 21 | 43 |
| | targeted | | tetracosenoic | | | | | |
| | | | acid (preferably | | | | | |
| | | | 2-hydroxy- | | | | | |
| | | | nervonic acid) | | | | | |
| 4102 | non- | At4g33040 | cerebronic | ARA_LEAF | p-PcUBI | GC | 25 | 58 |
| | targeted | | acid | | | | | |
| 4102 | non- | At4g33040 | hexadecadienoic | ARA_LEAF | p-PcUBI | GC | 20 | 61 |
| | targeted | | acid | | | | | |
| 4102 | non- | At4g33040 | hexadecatrienoic | ARA_LEAF | p-PcUBI | GC | 13 | 35 |
| | targeted | | acid | | | | | |
| 4348 | non- | At4g35310 | 2-hydroxy- | ARA_LEAF | p-PcUBI | GC | 32 | 125 |
| | targeted | | behenic acid | | | | | |
| 4348 | non- | At4g35310 | 2-hydroxy- | ARA_LEAF | p-PcUBI | GC | 18 | 97 |
| | targeted | | palmitic acid | | | | | |
| 122565 | non- | At5g16230 | oleic acid | ARA_LEAF | p-PcUBI | GC | 137 | 225 |
| | targeted | | | | | | | |
| 4904 | non- | At5g18600 | hexadecatrienoic | ARA_LEAF | p-PcUBI | GC | 17 | 37 |
| | targeted | | acid | | | | | |
| 5318 | non- | At5g57050 | hexadecatrienoic | ARA_LEAF | p-PcUBI | GC | 15 | 33 |
| | targeted | | acid | | | | | |
| 122803 | non- | At5g66710 | hexadecadienoic | ARA_LEAF | p-PcUBI | GC | 29 | 120 |
| | targeted | | acid | | | | | |
| 70583 | non- | AvinDRAFT_0035 | cerebronic | ARA_LEAF | p-PcUBI | GC | 22 | 38 |
| | targeted | | acid | | | | | |
| 70583 | non- | AvinDRAFT_0035 | 2-hydroxy- | ARA_LEAF | p-PcUBI | GC | 29 | 56 |
| | targeted | | behenic acid | | | | | |
| 70583 | non- | AvinDRAFT_0035 | 2-hydroxy- | ARA_LEAF | p-PcUBI | GC | 28 | 48 |
| | targeted | | tetracosenoic | | | | | |
| | | | acid | | | | | |
| | | | (preferably 2- | | | | | |
| | | | hydroxy- | | | | | |
| 70583 | non- | AvinDRAFT_0035 | [symbol] cid) | ARA_LEAF | p-PcUBI | GC | 20 | 53 |
| | targeted | | palmitic acid | | | | | |
| 26196 | non- | AvinDRAFT_1624 | hexadecadienoic | ARA_LEAF | p-PcUBI | GC | 22 | 50 |
| | targeted | | acid | | | | | |
| 114198 | non- | Avin- | 2-hydroxy- | ARA_LEAF | p-PcUBI | GC | 24 | 38 |
| | targeted | DRAFT_2010 | tetracosenoic | | | | | |
| | | | acid (preferably | | | | | |
| | | | 2-hydroxy- | | | | | |
| | | | nervonic acid) | | | | | |
| 6040 | non- | AvinDRAFT_2091 | 2-hydroxy- | ARA_LEAF | p-PcUBI | GC | 24 | 44 |
| | targeted | | palmitic acid | | | | | |
| 71325 | non- | AvinDRAFT_2365 | hexadecadienoic | ARA_LEAF | p-PcUBI | GC | 38 | 99 |
| | targeted | | acid | | | | | |
| 28040 | non- | AvinDRAFT_2754 | 2-hydroxy- | ARA_LEAF | p-PcUBI | GC | 28 | 72 |
| | targeted | | behenic acid | | | | | |
| 122948 | non- | AvinDRAFT_2827 | 2-hydroxy- | ARA_LEAF | p-PcUBI | GC | 23 | 80 |
| | targeted | | tetracosenoic | | | | | |
| | | | acid | | | | | |
| | | | (preferably 2- | | | | | |
| | | | hydroxy- | | | | | |
| 29500 | non- | AvinDRAFT_3253 | [symbol] cid) | ARA_LEAF | p-PcUBI | GC | 26 | 56 |
| | targeted | | tetracosenoic | | | | | |
| | | | acid | | | | | |
| | | | (preferably 2- | | | | | |
| | | | hydroxy- | | | | | |
| 114231 | non- | AvinDRAFT_3629 | he [symbol] | ARA_LEAF | p-PcUBI | GC | 23 | 113 |
| | targeted | | oic acid | | | | | |
| 72520 | non- | AVINDRAFT_4128 | lauric acid | ARA_LEAF | p-PcUBI | GC | 44 | 99 |
| | targeted | | | | | | | |
| 6510 | non- | AvinDRAFT_5103 | 2-hydroxy- | ARA_LEAF | p-PcUBI | GC | 20 | 76 |
| | targeted | | palmitic acid | | | | | |
| 32037 | non- | AvinDRAFT_5246 | 2-hydroxy- | ARA_LEAF | p-PcUBI | GC | 21 | 40 |
| | targeted | | palmitic acid | | | | | |
| 32308 | non- | AvinDRAFT_5292 | 2-hydroxy- | ARA_LEAF | p-PcUBI | GC | 64 | 74 |
| | targeted | | behenic acid | | | | | |
| 123087 | non- | AvinDRAFT_5466 | 2-hydroxy- | ARA_LEAF | p-PcUBI | GC | 22 | 55 |
| | targeted | | tetracosenoic | | | | | |
| | | | acid | | | | | |
| | | | (preferably 2- | | | | | |
| | | | hydroxy- | | | | | |
| | | | nervonic acid) | | | | | |
| 32648 | non- | Avin- | 2-hydroxy- | ARA_LEAF | p-PcUBI | GC | 22 | 32 |
| | targeted | DRAFT_5467 | tetracosenoic | | | | | |
| | | | acid (preferably | | | | | |
| | | | 2-hydroxy- | | | | | |
| | | | nervonic acid) | | | | | |
| 34044 | non- | AvinDRAFT_6700 | 2-hydroxy- | ARA_LEAF | p-PcUBI | GC | 25 | 48 |
| | targeted | | tetracosenoic | | | | | |
| | | | acid | | | | | |
| | | | (preferably 2- | | | | | |
| | | | hydroxy- | | | | | |
| 103958 | non- | B0050 | [symbol] cid) | ARA_LEAF | p-Super | GC | 33 | 55 |
| | targeted | | behenic acid | | | | | |
| 103958 | non- | B0050 | 2-hydroxy- | ARA_LEAF | p-Super | GC | 26 | 38 |
| | targeted | | tetracosenoic | | | | | |
| | | | acid | | | | | |
| | | | (preferably 2- | | | | | |
| | | | hydroxy- | | | | | |
| 123473 | non- | B0249 | [symbol] cid) | ARA_LEAF | p-Super | GC | 27 | 48 |
| | targeted | | acid | | | | | |
| 121111 | non- | B0312 | behenic acid | ARA_SEED_2 | p-USP | GC | 11 | 14 |
| | targeted | | | | | | | |
| 121111 | non- | B0312 | eicosanoic | ARA_SEED_2 | p-USP | GC | 11 | 14 |
| | targeted | | acid | | | | | |
| 7269 | plastidic | B0348 | myristic acid | ARA_SEED_2 | p-USP | GC | 21 | 39 |
| 7269 | plastidic | B0348 | oleic acid | ARA_SEED_2 | p-USP | GC | 24 | 43 |
| 121637 | non- | B0371 | hexadecatrienoic | ARA_LEAF | p-Super | GC | 17 | 32 |
| | targeted | | acid | | | | | |
| 7333 | non- | B0449 | cerebronic | ARA_LEAF | p-Super | GC | 24 | 205 |
| | targeted | | acid | | | | | |
| 7333 | non- | B0449 | 2-hydroxy- | ARA_LEAF | p-Super | GC | 28 | 193 |
| | targeted | | behenic acid | | | | | |
| 7333 | non- | B0449 | 2-hydroxy- | ARA_LEAF | p-Super | GC | 19 | 207 |
| | targeted | | palmitic acid | | | | | |
| 35936 | non- | B0518 | hexadecadienoic | ARA_LEAF | p-Super | GC | 25 | 64 |
| | targeted | | acid | | | | | |
| 93678 | non- | B0695 | 2-hydroxy- | ARA_LEAF | p-Super | GC | 38 | 63 |
| | targeted | | behenic acid | | | | | |
| 93678 | non- | B0695 | 2-hydroxy- | ARA_LEAF | p-Super | GC | 24 | 82 |
| | targeted | | tetracosenoic | | | | | |
| | | | acid (preferably | | | | | |
| | | | 2-hydroxy- | | | | | |
| | | | nervonic acid) | | | | | |
| 123498 | plastidic | B0720 | cerebronic | ARA_LEAF | p-Super | GC | 22 | 61 |
| | | | acid | | | | | |
| 75807 | non- | B0801 | 2-hydroxy- | ARA_LEAF | p-Super | GC | 23 | 41 |
| | targeted | | tetracosenoic | | | | | |
| | | | acid | | | | | |
| | | | (preferably 2- | | | | | |
| | | | hydroxy- | | | | | |
| 124051 | plastidic | B1046 | [symbol] cid) | ARA_LEAF | p-Super | GC | 24 | 69 |
| | | | acid | | | | | |
| 124127 | plastidic | B1092 | hexadecatrienoic | ARA_LEAF | p-Super | GC | 12 | 21 |
| | | | acid | | | | | |
| 118371 | non- | B1093 | hexadecadienoic | ARA_LEAF | p-Super | GC | 23 | 30 |
| | targeted | | acid | | | | | |
| 118371 | non- | B1093 | hexadecatrienoic | ARA_LEAF | p-Super | GC | 12 | 18 |
| | targeted | | acid | | | | | |
| 114552 | plastidic | B1095 | hexadecatrienoic | ARA_LEAF | p-Super | GC | 15 | 44 |
| | | | acid | | | | | |
| 114552 | plastidic | B1095 | 2-hydroxy- | ARA_LEAF | p-Super | GC | 27 | 73 |
| | | | behenic acid | | | | | |
| 37400 | non- | B1186 | oleic acid | ARA_LEAF | p-Super | GC | 26 | 110 |
| | targeted | | | | | | | |
| 37400 | non- | B1186 | 2-hydroxy- | ARA_LEAF | p-Super | GC | 19 | 45 |
| | targeted | | palmitic acid | | | | | |
| 124509 | plastidic | B1209 | behenic acid | ARA_SEED_2 | p-USP | GC | 9 | 16 |
| 98778 | plastidic | B1249 | behenic acid | ARA_SEED_2 | p-USP | GC | 9 | 15 |
| 98778 | plastidic | B1249 | heptadecanoic | ARA_SEED_2 | p-USP | GC | 13 | 25 |
| | | | acid | | | | | |
| 99188 | plastidic | B1346 | hexadecatrienoic | ARA_LEAF | p-Super | GC | 15 | 43 |
| | | | acid | | | | | |
| 124557 | non- | B1356 | lauric acid | ARA_LEAF | p-Super | GC | 45 | 61 |
| | targeted | | | | | | | |
| 38266 | plastidic | B1431 | 2-hydroxy- | ARA_LEAF | p-Super | GC | 25 | 94 |
| | | | tetracosenoic | | | | | |
| | | | acid | | | | | |
| | | | (preferably 2- | | | | | |
| | | | hydroxy- | | | | | |
| 115056 | non- | B1470 | he [symbol] | ARA_LEAF | p-Super | GC | 16 | 25 |
| | targeted | | oic acid | | | | | |
| 77774 | non- | B1479 | hexadecatrienoic | ARA_SEED_2 | p-USP | GC | 17 | 49 |
| | targeted | | acid | | | | | |
| 7947 | non- | B1522 | 2-hydroxy- | ARA_LEAF | p-Super | GC | 26 | 81 |
| | targeted | | behenic acid | | | | | |
| 38300 | non- | B1597 | hexadecatrienoic | ARA_LEAF | p-Super | GC | 12 | 48 |
| | targeted | | acid | | | | | |
| 7992 | non- | B1601 | 2-hydroxy- | ARA_LEAF | p-Super | GC | 22 | 43 |
| | targeted | | tetracosenoic | | | | | |
| | | | acid | | | | | |
| | | | (preferably 2- | | | | | |
| | | | hydroxy- | | | | | |
| 124571 | plastidic | B1621 | [symbol] cid) | ARA_LEAF | p-Super | GC | 25 | 35 |
| | | | tetracosenoic | | | | | |
| | | | acid | | | | | |
| | | | (preferably 2- | | | | | |
| | | | hydroxy- | | | | | |
| 38345 | plastidic | B1627 | [symbol] cid) | ARA_LEAF | p-Super | GC | 30 | 36 |
| | | | behenic acid | | | | | |
| 124805 | plastidic | B1636 | hexadecatrienoic | ARA_SEED_2 | p-USP | GC | 17 | 28 |
| | | | acid | | | | | |
| 78753 | non- | B1670 | 2-hydroxy- | ARA_LEAF | p-Super | GC | 23 | 63 |
| | targeted | | tetracosenoic | | | | | |
| | | | acid | | | | | |
| | | | (preferably 2- | | | | | |
| | | | hydroxy- | | | | | |
| 124907 | non- | B1703 | he [symbol] | ARA_LEAF | p-Super | GC | 28 | 66 |
| | targeted | | oic acid | | | | | |
| 124907 | non- | B1703 | hexadecatrienoic | ARA_LEAF | p-Super | GC | 14 | 24 |
| | targeted | | acid | | | | | |
| 78883 | non- | B1791 | hexadecadienoic | ARA_LEAF | p-Super | GC | 46 | 64 |
| | targeted | | acid | | | | | |
| 78953 | non- | B1837 | hexadecadienoic | ARA_LEAF | p-Super | GC | 31 | 35 |
| | targeted | | acid | | | | | |
| 78953 | non- | B1837 | hexadecatrienoic | ARA_LEAF | p-Super | GC | 15 | 27 |
| | targeted | | acid | | | | | |
| 8920 | non- | B2032 | hexadecadienoic | ARA_LEAF | p-Super | GC | 57 | 88 |
| | targeted | | acid | | | | | |
| 115595 | non- | B2099 | hexadecadienoic | ARA_LEAF | p-Super | GC | 36 | 47 |
| | targeted | | acid | | | | | |
| 115595 | non- | B2099 | hexadecatrienoic | ARA_LEAF | p-Super | GC | 18 | 24 |
| | targeted | | acid | | | | | |
| 39002 | non- | B2107 | 2-hydroxy- | ARA_LEAF | p-Super | GC | 24 | 39 |
| | targeted | | tetracosenoic | | | | | |
| | | | acid (preferably | | | | | |
| | | | 2-hydroxy- | | | | | |
| | | | nervonic acid) | | | | | |
| 124992 | plastidic | B2126 | lauric acid | ARA_LEAF | p-Super | GC | 32 | 43 |
| 125085 | plastidic | B2139 | 2-hydroxy- | ARA_LEAF | p-Super | GC | 20 | 27 |
| | | | palmitic acid | | | | | |
| 39040 | non- | B2178 | hexadecatrienoic | ARA_LEAF | p-Super | GC | 17 | 34 |
| | targeted | | acid | | | | | |
| 125105 | plastidic | B2216 | 2-hydroxy- | ARA_LEAF | p-Super | GC | 24 | 34 |
| | | | behenic acid | | | | | |
| 39219 | non- | B2360 | 2-hydroxy- | ARA_LEAF | p-Super | GC | 33 | 57 |
| | targeted | | tetracosenoic | | | | | |
| | | | acid | | | | | |
| | | | (preferably 2- | | | | | |
| | | | hydroxy- | | | | | |
| 39300 | non- | B2414 | [symbol] cid) | ARA_LEAF | p-Super | GC | 31 | 60 |
| | targeted | | tetracosenoic | | | | | |
| | | | acid | | | | | |
| | | | (preferably 2- | | | | | |
| | | | hydroxy- | | | | | |
| 115628 | non- | B2439 | he [symbol] | ARA_LEAF | p-Super | GC | 32 | 135 |
| | targeted | | oic acid | | | | | |
| 115628 | non- | B2439 | hexadecatrienoic | ARA_LEAF | p-Super | GC | 14 | 27 |
| | targeted | | acid | | | | | |
| 115628 | non- | B2439 | oleic acid | ARA_LEAF | p-Super | GC | 42 | 55 |
| | targeted | | | | | | | |
| 40329 | non- | B2474 | cerebronic | ARA_LEAF | p-Super | GC | 25 | 44 |
| | targeted | | acid | | | | | |
| 40329 | non- | B2474 | hexadecatrienoic | ARA_LEAF | p-Super | GC | 20 | 31 |
| | targeted | | acid | | | | | |
| 40329 | non- | B2474 | 2-hydroxy- | ARA_LEAF | p-Super | GC | 27 | 43 |
| | targeted | | tetracosenoic | | | | | |
| | | | acid | | | | | |
| | | | (preferably 2- | | | | | |
| | | | hydroxy- | | | | | |
| 9167 | non- | B2513 | he [symbol] | ARA_LEAF | p-Super | GC | 26 | 81 |
| | targeted | | oic acid | | | | | |
| 125142 | non- | B2585 | hexadecadienoic | ARA_LEAF | p-Super | GC | 21 | 32 |
| | targeted | | acid | | | | | |
| 125226 | non- | B2654 | 2-hydroxy- | ARA_LEAF | p-Super | GC | 38 | 41 |
| | targeted | | tetracosenoic | | | | | |
| | | | acid | | | | | |
| | | | (preferably 2- | | | | | |
| | | | hydroxy- | | | | | |
| 81075 | non- | B2747 | he [symbol] | ARA_LEAF | p-Super | GC | 13 | 45 |
| | targeted | | noic acid | | | | | |
| 125234 | non- | B2753 | eicosanoic | ARA_SEED_2 | p-USP | GC | 10 | 22 |
| | targeted | | acid | | | | | |
| 41732 | plastidic | B2963 | 2-hydroxy- | ARA_LEAF | p-Super | GC | 25 | 55 |
| | | | tetracosenoic | | | | | |
| | | | acid | | | | | |
| | | | (preferably 2- | | | | | |
| | | | hydroxy- | | | | | |
| 94380 | non- | B3442 | he [symbol] | ARA_LEAF | p-Super | GC | 26 | 102 |
| | targeted | | oic acid | | | | | |
| 125264 | plastidic | B3568 | cerebronic | ARA_LEAF | p-Super | GC | 30 | 44 |
| | | | acid | | | | | |
| 125487 | non- | B3812 | 2-hydroxy- | ARA_LEAF | p-Super | GC | 29 | 65 |
| | targeted | | behenic acid | | | | | |
| 100675 | non- | B3845 | hexadecadienoic | ARA_LEAF | p-Super | GC | 20 | 36 |
| | targeted | | acid | | | | | |
| 100675 | plastidic | B3845 | oleic acid | ARA_SEED_2 | p-USP | GC | 26 | 30 |
| 83821 | plastidic | B3872 | hexadecadienoic | ARA_LEAF | p-Super | GC | 28 | 58 |
| | | | acid | | | | | |
| 125541 | non- | B3925 | hexadecadienoic | ARA_LEAF | p-Super | GC | 25 | 66 |
| | targeted | | acid | | | | | |
| 125795 | plastidic | B3939 | behenic acid | ARA_SEED_2 | p-USP | GC | 11 | 25 |
| 125795 | plastidic | B3939 | heptadecanoic | ARA_SEED_2 | p-USP | GC | 22 | 33 |
| | | | acid | | | | | |
| 125795 | plastidic | B3939 | oleic acid | ARA_SEED_2 | p-USP | GC | 25 | 36 |
| 126377 | plastidic | B3944 | 2-hydroxy- | ARA_LEAF | p-Super | GC | 19 | 37 |
| | | | palmitic acid | | | | | |
| 126415 | plastidic | B4017 | 2-hydroxy- | ARA_LEAF | p-Super | GC | 29 | 49 |
| | | | behenic acid | | | | | |
| 119209 | non- | B4023 | hexadecadienoic | ARA_LEAF | p-Super | GC | 24 | 57 |
| | targeted | | acid | | | | | |
| 126421 | plastidic | B4069 | heptadecanoic | ARA_SEED_2 | p-USP | GC | 14 | 35 |
| | | | acid | | | | | |
| 126975 | plastidic | B4205 | 2-hydroxy- | ARA_LEAF | p-Super | GC | 20 | 34 |
| | | | palmitic acid | | | | | |
| 126991 | non- | C_pp032007025r | 2-hydroxy- | ARA_LEAF | p-Super | GC | 31 | 52 |
| | targeted | | behenic acid | | | | | |
| 10811 | non- | GM02- | 2-hydroxy- | ARA_LEAF | p-PcUBI | GC | 23 | 48 |
| | targeted | LC12622 | palmitic acid | | | | | |
| 46751 | non- | GM02- | cerebronic | ARA_LEAF | p-PcUBI | GC | 24 | 57 |
| | targeted | LC19289 | acid | | | | | |
| 46751 | non- | GM02- | hexadecatrienoic | ARA_LEAF | p-PcUBI | GC | 12 | 50 |
| | targeted | LC19289 | acid | | | | | |
| 116312 | non- | GM02- | 2-hydroxy- | ARA_LEAF | p-PcUBI | GC | 22 | 46 |
| | targeted | LC38418 | tetracosenoic | | | | | |
| | | | acid | | | | | |
| | | | (preferably 2- | | | | | |
| | | | hydroxy- | | | | | |
| 84865 | plastidic | Sll0281 | he [symbol] ) | ARA_LEAF | p-PcUBI | GC | 50 | 62 |
| | | | oic acid | | | | | |
| 106920 | mitochondrial | Sll0368 | 2-hydroxy- | ARA_LEAF | p-PcUBI | GC | 23 | 69 |
| | | | tetracosenoic | | | | | |
| | | | acid | | | | | |
| | | | (preferably 2- | | | | | |
| | | | hydroxy- | | | | | |
| 127103 | non- | Sll0406 | [symbol] cid) | ARA_LEAF | p-PcUBI | GC | 25 | 43 |
| | targeted | | tetracosenoic | | | | | |
| | | | acid | | | | | |
| | | | (preferably 2- | | | | | |
| | | | hydroxy- | | | | | |
| 101707 | non- | Sll0833 | [symbol] cid) | ARA_LEAF | p-PcUBI | GC | 28 | 41 |
| | targeted | | acid | | | | | |
| 127109 | non- | Sll0895 | cerebronic | ARA_LEAF | p-PcUBI | GC | 24 | 35 |
| | targeted | | acid | | | | | |
| 127109 | non- | Sll0895 | 2-hydroxy- | ARA_LEAF | p-PcUBI | GC | 22 | 40 |
| | targeted | | tetracosenoic | | | | | |
| | | | acid | | | | | |
| | | | (preferably 2- | | | | | |
| | | | hydroxy- | | | | | |
| 52634 | mitochondrial | Sll1031 | he [symbol] ) | ARA_LEAF | p-PcUBI | GC | 21 | 46 |
| | | | oic acid | | | | | |
| 52634 | mitochondrial | Sll1031 | hexadecatrienoic | ARA_LEAF | p-PcUBI | GC | 12 | 44 |
| | | | acid | | | | | |
| 11471 | mitochondrial | Sll1917 | cerebronic | ARA_LEAF | p-PcUBI | GC | 23 | 65 |
| | | | acid | | | | | |
| 11471 | mitochondrial | Sll1917 | 2-hydroxy- | ARA_LEAF | p-PcUBI | GC | 27 | 73 |
| | | | tetracosenoic | | | | | |
| | | | acid | | | | | |
| | | | (preferably 2- | | | | | |
| | | | hydroxy- | | | | | |
| 11990 | non- | Slr0338 | [symbol] cid) | ARA_LEAF | p-PcUBI | GC | 18 | 30 |
| | targeted | | palmitic acid | | | | | |
| 56153 | plastidic | Slr0597 | hexadecatrienoic | ARA_LEAF | p-PcUBI | GC | 21 | 26 |
| | | | acid | | | | | |
| 87397 | plastidic | Slr0721 | cerebronic | ARA_LEAF | p-PcUBI | GC | 24 | 37 |
| | | | acid | | | | | |
| 127183 | mitochondrial | Slr0879 | hexadecatrienoic | ARA_LEAF | p-PcUBI | GC | 17 | 38 |
| | | | acid | | | | | |
| 119222 | mitochondrial | Slr0949 | 2-hydroxy- | ARA_LEAF | p-PcUBI | GC | 21 | 92 |
| | | | tetracosenoic | | | | | |
| | | | acid | | | | | |
| | | | (preferably 2- | | | | | |
| | | | hydroxy- | | | | | |
| 87655 | plastidic | Slr0966 | he [symbol] ) | ARA_LEAF | p-PcUBI | GC | 35 | 194 |
| | | | oic acid | | | | | |
| 57734 | non- | Slr1096 | 2-hydroxy- | ARA_LEAF | p-PcUBI | GC | 31 | 55 |
| | targeted | | behenic acid | | | | | |
| 116460 | mitochondrial | Slr1250 | 2-hydroxy- | ARA_LEAF | p-PcUBI | GC | 19 | 38 |
| | | | palmitic acid | | | | | |
| 127521 | mitochondrial | Slr2035 | hexadecatrienoic | ARA_LEAF | p-PcUBI | GC | 12 | 23 |
| | | | acid | | | | | |
| 12341 | mitochondrial | Slr2072 | cerebronic | ARA_LEAF | p-PcUBI | GC | 30 | 42 |
| | | | acid | | | | | |
| 12341 | mitochondrial | Slr2072 | hexadecatrienoic | ARA_LEAF | p-PcUBI | GC | 15 | 59 |
| | | | acid | | | | | |
| 12341 | plastidic | Slr2072 | 2-hydroxy- | ARA_LEAF | p-PcUBI | GC | 27 | 38 |
| | | | tetracosenoic | | | | | |
| | | | acid | | | | | |
| | | | (preferably 2- | | | | | |
| | | | hydroxy- | | | | | |
| 128024 | plastidic | Ssl3044 | [symbol] cid) | ARA_LEAF | p-PcUBI | GC | 22 | 37 |
| | | | tetracosenoic | | | | | |
| | | | acid | | | | | |
| | | | (preferably 2- | | | | | |
| | | | hydroxy- | | | | | |
| 12698 | non- | TTC0019 | [symbol] cid) | ARA_LEAF | p-PcUBI | GC | 31 | 39 |
| | targeted | | behenic acid | | | | | |
| 61070 | non- | TTC0337 | 2-hydroxy- | ARA_LEAF | p-PcUBI | GC | 30 | 53 |
| | targeted | | tetracosenoic | | | | | |
| | | | acid | | | | | |
| | | | (preferably 2- | | | | | |
| | | | hydroxy- | | | | | |
| 61553 | non- | TTC0917 | [symbol] cid) | ARA_LEAF | p-PcUBI | GC | 27 | 89 |
| | targeted | | acid | | | | | |
| 61553 | non- | TTC0917 | hexadecadienoic | ARA_LEAF | p-PcUBI | GC | 22 | 139 |
| | targeted | | acid | | | | | |
| 61723 | non- | TTC1193 | 2-hydroxy- | ARA_LEAF | p-PcUBI | GC | 28 | 35 |
| | targeted | | behenic acid | | | | | |
| 62079 | non- | TTC1386 | hexadecatrienoic | ARA_LEAF | p-PcUBI | GC | 14 | 26 |
| | targeted | | acid | | | | | |
| 12974 | non- | TTC1550 | 2-hydroxy- | ARA_LEAF | p-PcUBI | GC | 26 | 57 |
| | targeted | | behenic acid | | | | | |
| 128139 | non- | Yar019c | hexadecadienoic | ARA_LEAF | Big35S | GC | 21 | 25 |
| | targeted | | acid | | | | | |
| 128139 | non- | Yar019c | hexadecatrienoic | ARA_LEAF | Big35S | GC | 13 | 20 |
| | targeted | | acid | | | | | |
| 62524 | non- | Ybl021c | hexadecadienoic | ARA_LEAF | Big35S | GC | 21 | 96 |
| | targeted | | acid | | | | | |
| 62524 | non- | Ybl021c | hexadecatrienoic | ARA_LEAF | Big35S | GC | 12 | 23 |
| | targeted | | acid | | | | | |
| 62717 | non- | Ybr160w | hexadecatrienoic | ARA_LEAF | Big35S | GC | 13 | 54 |
| | targeted | | acid | | | | | |
| 96980 | plastidic | Ycl027w | cerebronic | ARA_LEAF | p-Super | GC | 25 | 46 |
| | | | acid | | | | | |
| 96980 | plastidic | Ycl027w | 2-hydroxy- | ARA_LEAF | p-Super | GC | 25 | 41 |
| | | | tetracosenoic | | | | | |
| | | | acid | | | | | |
| | | | (preferably 2- | | | | | |
| | | | hydroxy- | | | | | |
| 113220 | plastidic | Ycl049c | [symbol] cid) | ARA_LEAF | p-Super | GC | 22 | 74 |
| | | | palmitic acid | | | | | |
| 119408 | non- | Ydl022w | oleic acid | ARA_SEED_2 | p-PcUBI | GC | 34 | 156 |
| | targeted | | | | | | | |
| 122228 | non- | Ydl066w | eicosanoic | ARA_SEED_2 | p-PcUBI | GC | 8 | 17 |
| | targeted | | acid | | | | | |
| 122228 | non- | Ydl066w | heptadecanoic | ARA_SEED_2 | p-PcUBI | GC | 16 | 20 |
| | targeted | | acid | | | | | |
| 120045 | non- | Ydl126c | hexadecadienoic | ARA_LEAF | Big35S | GC | 30 | 63 |
| | targeted | | acid | | | | | |
| 120045 | non- | Ydl126c | hexadecatrienoic | ARA_LEAF | Big35S | GC | 14 | 34 |
| | targeted | | acid | | | | | |
| 113224 | plastidic | Ydl131w | 2-hydroxy- | ARA_LEAF | p-Super | GC | 29 | 50 |
| | | | behenic acid | | | | | |
| 113330 | plastidic | Ydr035w | behenic acid | ARA_LEAF | p-Super | GC | 38 | 128 |
| 128148 | plastidic | Ydr077w | 2-hydroxy- | ARA_LEAF | p-Super | GC | 22 | 32 |
| | | | tetracosenoic | | | | | |
| | | | acid (preferably | | | | | |
| | | | 2-hydroxy- | | | | | |
| | | | nervonic acid) | | | | | |
| 14706 | plastidic | Ydr131c | 2-hydroxy- | ARA_LEAF | p-Super | GC | 26 | 66 |
| | | | tetracosenoic | | | | | |
| | | | acid | | | | | |
| | | | (preferably 2- | | | | | |
| | | | hydroxy- | | | | | |
| 90159 | plastidic | Ydr273w | [symbol] cid) | ARA_LEAF | p-Super | GC | 27 | 30 |
| | | | acid | | | | | |
| 90159 | plastidic | Ydr273w | 2-hydroxy- | ARA_LEAF | p-Super | GC | 24 | 28 |
| | | | tetracosenoic | | | | | |
| | | | acid | | | | | |
| | | | (preferably 2- | | | | | |
| | | | hydroxy- | | | | | |
| 120241 | plastidic | Ydr339c | he [symbol] ) | ARA_LEAF | p-Super | GC | 14 | 20 |
| | | | oic acid | | | | | |
| 128511 | plastidic | Yel046c | heptadecanoic | ARA_SEED_2 | p-USP | GC | 20 | 35 |
| | | | acid | | | | | |
| 113598 | non- | Yer112w | myristic acid | ARA_LEAF | Big35S | GC | 46 | 90 |
| | targeted | | | | | | | |
| 63807 | non- | Yfl016c | hexadecadienoic | ARA_LEAF | Big35S | GC | 20 | 48 |
| | targeted | | acid | | | | | |
| 63807 | non- | Yfl016c | hexadecatrienoic | ARA_LEAF | Big35S | GC | 13 | 39 |
| | targeted | | acid | | | | | |
| 64144 | non- | Yfl019c | 2-hydroxy- | ARA_LEAF | Big35S | GC | 31 | 108 |
| | targeted | | behenic acid | | | | | |
| 64144 | non- | Yfl019c | 2-hydroxy- | ARA_LEAF | Big35S | GC | 21 | 75 |
| | targeted | | palmitic acid | | | | | |
| 90189 | plastidic | Yfl030w | hexadecadienoic | ARA_LEAF | p-Super | GC | 31 | 53 |
| | | | acid | | | | | |
| 64177 | non- | Ygl237c | hexadecatrienoic | ARA_LEAF | Big35S | GC | 13 | 35 |
| | targeted | | acid | | | | | |
| 117482 | non- | Ygr104c | eicosanoic | ARA_SEED_2 | p-PcUBI | GC | 11 | 19 |
| | targeted | | acid | | | | | |
| 64218 | plastidic | Ygr175c | cerebronic | ARA_LEAF | p-PcUBI | GC | 24 | 34 |
| | | | acid | | | | | |
| 128655 | plastidic | Ygr286c | hexadecadienoic | ARA_LEAF | p-Super | GC | 23 | 46 |
| | | | acid | | | | | |
| 128967 | plastidic | Yhr033w | cerebronic | ARA_LEAF | p-PcUBI | GC | 21 | 27 |
| | | | acid | | | | | |
| 64964 | plastidic | Yjl073w | cerebronic | ARA_LEAF | p-Super | GC | 30 | 45 |
| | | | acid | | | | | |
| 97631 | non- | Yjl127w-a | 2-hydroxy- | ARA_LEAF | Big35S | GC | 26 | 40 |
| | targeted | | behenic acid | | | | | |
| 14843 | non- | Yjr130c | 2-hydroxy- | ARA_LEAF | Big35S | GC | 23 | 70 |
| | targeted | | tetracosenoic | | | | | |
| | | | acid (preferably | | | | | |
| | | | 2-hydroxy- | | | | | |
| | | | nervonic acid) | | | | | |
| 14843 | non- | Yjr130c | 2-hydroxy- | ARA_LEAF | Big35S | GC | 19 | 106 |
| | targeted | | palmitic acid | | | | | |
| 66274 | non- | Yjr153w | 2hydroxypalmitic | ARA_LEAF | p-PcUBI | GC | 18 | 22 |
| | targeted | | acid | | | | | |
| 129065 | non- | Ylr210w | hexadecatrienoic | ARA_LEAF | Big35S | GC | 13 | 23 |
| | targeted | | acid | | | | | |
| 129123 | plastidic | Ylr326w | hexadecadienoic | ARA_LEAF | p-Super | GC | 21 | 94 |
| | | | acid | | | | | |
| 129123 | plastidic | Ylr326w | hexadecatrienoic | ARA_LEAF | p-Super | GC | 13 | 35 |
| | | | acid | | | | | |
| 91781 | plastidic | Yml083c | 2-hydroxy- | ARA_LEAF | p-Super | GC | 28 | 56 |
| | | | behenic acid | | | | | |
| 15175 | plastidic | Yml084w | 2-hydroxy- | ARA_LEAF | p-Super | GC | 26 | 59 |
| | | | tetracosenoic | | | | | |
| | | | acid | | | | | |
| | | | (preferably 2- | | | | | |
| | | | hydroxy- | | | | | |
| 129141 | plastidic | Ymr186w | [symbol] cid) | ARA_LEAF | p-Super | GC | 18 | 27 |
| | | | palmitic acid | | | | | |
| 129470 | mitochondrial | Ynl130c | oleic acid | ARA_SEED_2 | p-USP | GC | 23 | 36 |
| 129546 | plastidic | Ynl321w | 2-hydroxy- | ARA_LEAF | p-Super | GC | 26 | 53 |
| | | | behenic acid | | | | | |
| 129574 | non- | Yor187w | hexadecatrienoic | ARA_LEAF | Big35S | GC | 17 | 27 |
| | targeted | | acid | | | | | |
| 118212 | plastidic | Ypl271w | 2-hydroxy- | ARA_LEAF | p-Super | GC | 25 | 75 |
| | | | tetracosenoic | | | | | |
| | | | acid | | | | | |
| | | | (preferably 2- | | | | | |
| | | | hydroxy- | | | | | |
| 130181 | plastidic | Ypr167c | [symbol] cid) | ARA_LEAF | p-PcUBI | GC | 66 | 84 |
| | | | palmitic acid | | | | | |
| 128161 | non- | YDR447C | oleic acid | ARA_LEAF | Big35S | GC | 25 | 395 |
| | targeted | | | | | | | |
| 125348 | non- | B3644 | 2-hydroxy- | ARA_LEAF | p-Super | GC | 2 | 77 |
| | targeted | | behenic acid | | | | | |
| 125348 | non- | B3644 | behenic acid | ARA_LEAF | p-Super | GC | 0 | 58 |
| | targeted | | | | | | | |
| 125348 | non- | B3644 | nervonic acid | ARA_LEAF | p-Super | GC | 12 | 319 |
| | targeted | | | | | | | |

[symbol]

indicates data missing or illegible when filed

Column 1 shows the SEQ ID NO, Column 2 shows the expression type (targeted or non-targeted), Column 3 shows the “gene name” (locus), Column 4 shows the metabolite analyzed, Column 5 indicates the A. thaliana source tissue analyzed, Column 6 indicates the used promoter for expression, Column 7 indicates the analytical method. Columns 8 and 9 show the minimum and the maximum increase of the analyzed metabolite (in percent) in comparison to the wild type (=ratio_by_weight, given as percent increase).

When the analyses were repeated independently, all results proved to be significant.

[0505.1.8.12] to [0515.1.8.12] for the disclosure of these paragraphs see [0505.1.8.8] to [0515.1.8.8] above.

In a further embodiment, the present invention relates in paragraphs [0000.1.7.13.] to [0514.1.7.13.] to a further process for the production of the fine chemical abscisic acid, cryptoxanthin, violaxanthin and/or zeaxanthin as defined below and corresponding embodiments as described herein as follows.

for the disclosure of this paragraph see [0001.1.7.7] above

Carotenoids are red, yellow and orange pigments that are widely distributed in nature. Although specific carotenoids have been identified in photosynthetic centers in plants, in bird feathers, in crustaceans and in marigold petals, they are especially abundant in yellow-orange fruits and vegetables and dark green, leafy vegetables. Of the more than 700 naturally occurring carotenoids identified thus far, as many as 50 may be absorbed and metabolized by the human body.

In animals some carotenoids (particularly beta-carotene) serve as dietary precursors to Vitamin A, and many of them may function as fat-soluble antioxidants. In plants carotenes serve for example as antioxidants to protect the highly reactive photosystems and act as accessory photopigments. In vitro experiments have shown that lycopene, alpha-carotene, zeaxanthin, lutein and cryptoxanthin quench singlet oxygen and inhibit lipid peroxidation. The isolation and identification of oxidized metabolites of lutein, zeaxanthin and lycopene provide direct evidence of the antioxidant action of these carotenoids.

Carotenoids are 40-carbon (C₄₀) terpenoids generally comprising eight isoprene (C₅) units joined together. Linking of the units is reversed at the center of the molecule. “Ketocarotenoid” is a general term for carotenoid pigments that contain a keto group in the ionene ring portion of the molecule, whereas “hydroxycarotenoid” refers to carotenoid pigments that contain a hydroxyl group in the ionene ring. Trivial names and abbreviations will be used throughout this disclosure, with IUPAC-recommended semi-systematic names usually being given in parentheses after first mention of a trivial name.

Carotenoids are synthesized from a five carbon atom metabolic precursor, isopentenyl pyrophosphate (IPP). There are at least two known biosynthetic pathways in the formation of IPP, the universal isoprene unit. One pathway begins with mevalonic acid, the first specific precursor of terpenoids, formed from acetyl-CoA via HMG-CoA (3-hydroxy-3-methylglutaryl-CoA), that is itself converted to isopentenyl pyrophosphate (IPP). Later, condensation of two geranylgeranyl pyrophosphate (GGPP) molecules with each other produces colorless phytoene, which is the initial carotenoid. Studies have also shown the existence of an alternative, mevalonate-independent pathway for IPP formation that was characterized initially in several species of eubacteria, a green alga, and in the plastids of higher plants. The first reaction in this alternative pathway is the transketolase-type condensation reaction of pyruvate and D-glyceraldehylde-3-phosphate to yield 1-deoxy-D-xylulose-5-phosphate (DXP) as an intermediate.

Through a series of desaturation reactions, phytoene is converted to phytofluene, ζ-carotene, neurosporene and finally to lycopene. Subsequently, lycopene is converted by a cyclization reaction to β-carotene that contains two [3-ionene rings. One or two hydroxyl group(s) are introduced into each ring of β-carotene to thereby synthesize [3-cryptoxanthin resp. zeaxanthin. By epoxidation of the two rings of zeaxanthin by beta.epoxidase violaxanthin is synthetized. Lycopene is also converted to alpha-carotene. The introduction of one hydroxyl group leads to alpha-cryptoxanthin. A breakdown product of the 9-cis-epoxycarotenoid xanthophylls, 9-cis-violaxanthin and 9′-cis-neoxanthin in green plants is abscisic acid.

Carotenoids absorb light in the 400-500 nm region of the visible spectrum. This physical property imparts the characteristic red/yellow color of the pigments. A conjugated backbone composed of isoprene units is usually inverted at the center of the molecule, imparting symmetry.

Changes in geometrical configuration about the double bonds result in the existence of many cis- and trans-isomers. Hydroxylated, oxidized, hydrogenated or ring-containing derivatives also exist. Hydrocarbon carotenoids are classified as carotenes while those containing oxygen are known as xanthophylls.

In animals, carotenoids are absorbed from the intestine with the aid of dietary fat and incorporated into chylomicrons for transport in the serum. The different structural features possessed by carotenoids account for selective distribution in organ tissue, biological activity and provitamin A potency, or in vivo conversion to vitamin A. Due to the hydrophobic character, carotenoids are associated with lipid portions of human tissues, cells, and membranes. In general, 80-85% of carotenoids are distributed in adipose tissue, with smaller amounts found in the liver, muscle, adrenal glands, and reproductive organs. Approximately 1% circulate in the serum on high and low density lipoproteins. Serum concentrations are fairly constant and slow to change during periods of low intake. The estimated half-life was estimated to be 11-14 days for lycopene, α-carotene, β-carotene, lutein and zeaxanthin. Evidence for the existence of more than one body pool has been published. The major serum carotenoids are α-carotene, β-carotene, lutein, zeaxanthin, lycopene and cryptoxanthin. Smaller amounts of polyenes such as phytoene and phytofluene are also present.

Human serum levels reflect lifestyle choices and dietary habits within and between cultures. Approximately only 15 circulate in the blood, on HDL and LDL. Variations can be attributed to different intakes, unequal abilities to absorb certain carotenoids, and different rates of metabolism and tissue uptake. Decreased serum levels occur with alcohol consumption, the use of oral contraceptives, smoking and prolonged exposure to UV light.

α-Carotene, β-carotene and β-cryptoxanthin can be converted to retinol or vitamin A in the intestine and liver by the enzyme 15-15′-b-carotenoid dioxygenase. Such in vivo formation of retinol appears to be homeostatically controlled, such that conversion to retinol is limited in persons having adequate vitamin A status.

The established efficacy of beta-carotene in quenching singlet oxygen and intercepting deleterious free radicals and reactive oxygen species makes it part of the diverse antioxidant defense system in humans. Reactive oxygen species have been implicated in the development of many diseases, including ischemic heart disease, various cancers, cataracts and macular degeneration. Because the conjugated polyene portion of beta-carotene confers its antioxidant capability and all carotenoids possess this structural feature, research efforts have been directed at evaluating the efficacy of other carotenoids in the prevention of free radical-mediated diseases. Indeed, in vitro experiments have demonstrated that lycopene, alphacarotene, zeaxanthin, lutein and cryptoxanthin quench singlet oxygen and inhibit lipid peroxidation. In addition to antioxidant capability, other biological actions of carotenoids include the ability to enhance immunocompetence and in vitro gap junction communication, reduce or inhibited mutagenesis and inhibit cell transformations in vitro.

Many epidemiological studies have established an inverse correlation between dietary intake of yellow-orange fruit and dark green, leafy vegetables and the incidence of various cancers, especially those of the mouth, pharynx, larynx, esophagus, lung, stomach, cervix and bladder.

While a number of protective compounds may be responsible for this observation, the coincidence of carotenoids in these foods has been noted. Because nutritionists and medical professionals currently recognize the occurrence of a large number of distinct carotenoids in food, interest in their functions and biological impact on health is burgeoning.

Lutein exists in the retina. It functions to protect photoreceptor cells from light-generated oxygen radicals, and thus plays a key role in preventing advanced macular degeneration. In addition to lutein, zeaxanthin exists in the retina and confers protection against macular degeneration. Zeaxanthin is also prevalent in ovaries and adipocyte tissue. This xanthophyll does not possess provitamin A activity.

Alcohol consumption has been shown to influence lipid peroxidation. Anhydrolutein, an oxidative by-product of lutein and zeaxanthin, was higher in plasma after alcohol ingestion, while concentrations of these xanthophylls were reduced. Lutein and zeaxanthin may therefore have protective effects against LDL oxidation.

Cryptoxanthin, also known as beta-cryptoxanthin, is a member of the carotenoid family, a group of flavonoids that provide color and flavor to fruits and vegetables. Like alpha- and betacarotene, cryptoxanthin is a powerful antioxidant that can be converted to active retinol, or vitamin A, in the body. Vitamin A is crucial to the maintenance of healthy vision, reproduction, and body tissues. Recent studies have shown that cryptoxanthin also plays an important role in preventing many forms of cancer. Cryptoxanthin may protect against cancer and other diseases associated with aging, including heart disease, skin cancer, prostate cancer, and arthritis. Like other carotenoids, cryptoxanthin almost certainly plays a key role in keeping the eyes healthy and preventing against age-related macular degeneration (AMD) and cataracts. Research also indicates that carotenoids may play a role in the prevention of many other serious health conditions, including Acquired Immunodeficiency Syndrome (AIDS), asthma, chronic yeast infection, and infertility. beta-Cryptoxanthin is capable of quenching singlet oxygen. beta-Cryptoxanthin is used to color butter. beta-Cryptoxanthin exhibits provitamin A activity.

The all-trans isomer of this carotenoid is the major source of dietary retinoids, due to its high provitamin A activity. One molecule of trans-beta-carotene can theoretically provide two molecules of trans retinaldehyde in vivo. alpha-Carotene is similar to beta-carotene in its biological activity, but quenches singlet oxygen more effectively.

Alpha-Carotene, beta-carotene and beta-cryptoxanthin can be converted to Vitamin A in the intestine and liver. Vitamin A is essential for the immune response and is also involved in other defenses against infectious agents. Nevertheless, in many individuals, this conversion is slow and ineffectual, particularly for older. Some individuals are known as non or low-responders because they do not convert beta-carotene to Vitamin A at the rate as expected. A number of factors can inhibit this conversion of beta-carotene to Vitamin A. The major reason why so many Americans have a poor vitamin A status is the regular use of excessive alcohol. Intestinal parasites can be a factor. And, any prescription drug that requires liver metabolism will decrease the liver conversion of beta-carotene to retinol in the liver. Diabetics and individuals with hypothyroidism or even borderline hypothyroidism are likely to be low-responders.

In plants, approximately 80-90% of the carotenoids present in green, leafy vegetables such as broccoli, kale, spinach and brussel sprouts are xanthophylls, whereas 10-20% are carotenes. Conversely, yellow and orange vegetables including carrots, sweet potatoes and squash contain predominantly carotenes. Up to 60% of the xanthophylls and 15% of the carotenes in these foods are destroyed during microwave cooking.

Dietary sources of Zeaxanthin include peaches, squash, apricots, oranges, papaya, prunes, pumpkin, mango, kale, kiwi, lettuce, honeydew melon and yellow corn.

Dietary sources of beta-Carotene include mango, cantaloupe, carrots, pumpkin, papaya, peaches, prunes, squash, sweet potato, apricots, cabbage, lima beans, green beans, broccoli, brussel sprouts, kale, kiwi, lettuce, peas, spinach, tomatoes, pink grapefruit, honeydew melon and oranges.

Some carotenoids occur particularly in a wide variety of marine animals including fish such as salmonids and sea bream, and crustaceans such as crab, lobster, and shrimp. Because animals generally cannot biosynthesize carotenoids, they obtain those carotenoids present in microorganisms or plants upon which they feed.

Carotenoids e.g. xanthophylls, e.g. as astaxanthin, supplied from biological sources, such as crustaceans, yeast, and green alga is limited by low yield and costly extraction methods when compared with that obtained by organic synthetic methods. Usual synthetic methods, however, produce by-products that can be considered unacceptable. It is therefore desirable to find a relatively inexpensive source of carotenoids, in particular xanthophylls, to be used as a feed supplement in aquaculture and as a valuable chemical for other industrial uses and for diets. Sources of xanthophylls include crustaceans such as a krill in the Antarctic Ocean, cultured products of the yeast Phaffia, cultured products of a green alga Haematococcus pluvialis, and products obtained by organic synthetic methods. However, when crustaceans such as a krill or the like are used, a great deal of work and expense are required for the isolation of xanthophylls from contaminants such as lipids and the like during the harvesting and extraction. Moreover, in the case of the cultured product of the yeast Phaffia, a great deal of expense is required for the gathering and extraction of astaxanthin because the yeast has rigid cell walls and produces xanthophylls only in a low yield. One approach to increase the productivity of some xanthophylls' production in a biological system is to use genetic engineering technology.

In many plants, lycopene is a branch point in carotenoid biosynthesis. Thus, some of the plant's lycopene is made into beta-carotene and zeaxanthin, and sometimes zeaxanthin diglucoside, whereas remaining portions of lycopene are formed into alpha-carotene and lutein (3,3′-dihydroxy-α-carotene), another hydroxylated compound. Carotenoids in higher plants; i.e., angiosperms, are found in plastids; i.e., chloroplasts and chromoplasts. Plastids are intracellular storage bodies that differ from vacuoles in being surrounded by a double membrane rather than a single membrane. Plastids such as chloroplasts can also contain their own DNA and ribosomes, can reproduce independently and synthesize some of their own proteins. Plastids thus share several characteristics of mitochondria. In leaves, carotenoids are usually present in the grana of chloroplasts where they provide a photoprotective function. Betacarotene and lutein are the predominant carotenoids, with the epoxidized carotenoids violaxanthin and neoxanthin being present in smaller amounts. Carotenoids accumulate in developing chromoplasts of flower petals, usually with the disappearance of chlorophyll. As in flower petals, carotenoids appear in fruit chromoplasts as they develop from chloroplasts. Most enzymes that take part in conversion of phytoene to carotenes and xanthophylls are labile, membrane-associated proteins that lose activity upon solubilization. In maize, cartonoids were present in horny endosperm (74% to 86%), floury endosperm (9%-23%) and in the germ and bran of the kernel.

At the present time only a few plants are widely used for commercial colored carotenoid production. However, the productivity of colored carotenoid synthesis in most of these plants is relatively low and the resulting carotenoids are expensively produced.

Dried marigold petals and marigold petal concentrates obtained from so-called xanthophyll marigolds are used as feed additives in the poultry industry to intensify the yellow color of egg yolks and broiler skin. The pigmenting ability of marigold petal meal resides largely in the carotenoid fraction known as the xanthophylls, primarily lutein esters. The xanthophyll zeaxanthin, also found in marigold petals, has been shown to be effective as a broiler pigmenter, producing a highly acceptable yellow to yellow-orange color. Of the xanthophylls, the pigments lutein and zeaxanthin are the most abundant in commercially available hybrids.

Carotenoids have been found in various higher plants in storage organs and in flower petals. For example, marigold flower petals accumulate large quantities of esterified lutein as their predominant xanthophyll carotenoid (about 75 to more than 90 percent), with smaller amounts of esterified zeaxanthin. Besides lutein and zeaxanthin, marigold flower petals also typically exhibit a small accumulation of β-carotene and epoxidized xanthophylls, but do not produce or accumulate canthaxanthin or astaxanthin because a 4-keto-β-ionene ring-forming enzyme is absent in naturally-occurring marigolds or their hybrids.

One way to increase the productive capacity of biosynthesis is to apply recombinant DNA technology. Thus, it would be desirable to produce colored carotenoids generally and, with the use of recent advances in determining carotenoid biosynthesis from β-carotene to xanthophylls and abscisic acid or from alpha-carotene to lutein to control the production of carotenoids. That type of production permits control over quality, quantity and selection of the most suitable and efficient producer organisms. The latter is especially important for commercial production economics and therefore availability to consumers.

Methods of recombinant DNA technology have been used for some years to improve the production of xanthophylls in microorganisms, in particular algae or in plants by amplifying individual xanthophyll biosynthesis genes and investigating the effect on xanthophyll production. It is for example reportet, that the five ketocarotenoids, e.g. the xanthophyll astaxanthin could be produced in the nectaries of transgenic tobacco plants. Those transgenic plants were prepared by Argobacterium tumifaciens-mediated transformation of tobacco plants using a vector that contained a ketolase-encoding gene from H. pluvialis denominated crtO along with the Pds gene from tomato as the promoter and to encode a leader sequence. The Pds gene was said by those workers to direct transcription and expression in chloroplasts and/or chromoplast containing tissues of plants. Those results indicated that about 75 percent of the carotenoids found in the flower of the transformed plant contained a keto group. Further, in maize the phytonene synthase (Psy), Phytone desaturase (Pds), and the ζ-carotene desaturase were identified and it was shown, that PSY activity is an important control point for the regulation of the flux. Genes suitable for conversion of microorganisms have also been reported (U.S. Pat. No. 6,150,130 WO 99/61652). Two different genes that can convert a carotenoid β-ionene ring compound into astaxanthin have been isolated from the green alga Haematococcus pluvialis. Zeaxanthin or ζ-carotene were also found in the marine bacteria Agrobacterium aurantiacum, Alcaligenes PC-1, Erwinia uredovora. An A. aurantiacum crtZ gene was introduced to an E. coli transformant that accumulated all-trans-β-carotene. The transformant so formed produced zeaxanthin. A gene cluster encoding the enzymes for a carotenoid biosynthesis pathway has been also cloned from the purple photosynthetic bacterium Rhodobacter capsulatus. A similar cluster for carotenoid biosynthesis from ubiquitous precursors such as farnesyl pyrophosphate and geranyl pyrophosphate has been cloned from the non-photosynthetic bacteria Erwinia herbicola.

Yet another carotenoid biosynthesis gene cluster has been cloned from Erwinia uredovora. It is yet unknown and unpredictable as to whether enzymes encoded by other organisms behave similarly to that of A. aurantiacum in vitro or in vivo after transformation into the cells of a higher plant.

The content of some carotinoids in a cell was also increased by increasing or decreasing certain activities as disclosed in WO2006/069610, WO2007/087815, WO2008/034648 or WO 2008/04505.

In addition to the above said about the biological importance of carotenoids, e.g. in vision, bone growth, reproduction, immune function, gene expression, emboryonic expression, cell division and cell differation, and respiration, it should be mentioned that in the world, the prevalence of vitamin A deficiency ranges from 100 to 250 million children and an estimated 250.000 to 500.000 children go blind each year from vitamin A deficiency.

Thus, it would be advantageous if an algae or other microorganism were available who produce large amounts of cryptoxanthin, zeaxanthin, violaxanthin or other carotenoids or abcisic acid.

Therefore improving the quality of foodstuffs and animal feeds is an important task of the food-and-feed industry. This is necessary since, for example, as mentioned above xanthophylls or abcisic acid, which occur in plants and some microorganisms are limited with regard to the supply of mammals. Especially advantageous for the quality of foodstuffs and animal feeds is as balanced as possible a carotenoids or abcisic acid profile in the diet since a great excess of some carotenoids or abcisic acid above a specific concentration in the food has only some positive effect. Efforts should be taken to optimize the levels of health-promoting compounds in foods. In order to do this, the compositional variation throughout the food chain must be known. The composition of carotenoids differs qualitatively and quantitatively in a given food due to factors such as cultivar or varietal differences, stage of maturity, climatic or geographic effects, part of the plant consumed, conditions during agricultural production, post-harvest handling, processing and storage. Based on this variability, measures must be taken to offer the population foods with higher and uniformly carotenoids and/or abcisic acid concentrations.

To ensure a high quality of foods and animal feeds, it is therefore necessary to add a plurality of carotenoids or abcisic acid in a balanced manner to suit the respective organism. Accordingly, there is still a great demand for new and more suitable genes, which encode enzymes or regulators, which participate in the biosynthesis of carotenoids or abcisic acid, in particular abscisic acid, cryptoxanthin, violaxanthin, and/or zeaxanthin and make it possible to produce certain carotenoids specifically on an industrial scale without unwanted byproducts being formed. In the selection of genes for biosynthesis or regulation two characteristics above all are particularly important. On the one hand, there is as ever a need for improved processes for obtaining the highest possible contents of carotenoids or abcisic acidand on the other hand as less as possible byproducts should be produced in the production process.

It is an object of the present invention to develop an inexpensive process for the synthesis of abscisic acid, cryptoxanthin, violaxanthin, and/or zeaxanthin.

It is a further object of the present invention to develop an inexpensive process for the synthesis of abscisic acid, cryptoxanthin, violaxanthin, and/or zeaxanthin and to assure that the abscisic acid, cryptoxanthin, violaxanthin, and/or zeaxanthin are more accessible and facilely to isolate and recover in an industrial scale from the producing organism, preferably from a plant.

for the disclosure of this paragraph see [0013.1.7.7] above

Accordingly, in a first embodiment, the invention relates to a process for the production of at least one fine chemical selected from the group consisting of: abscisic acid, cryptoxanthin, violaxanthin, and zeaxanthin, or, in other words, of the “fine chemical” or “fine chemical of the invention”.

The terms “fine chemical of the invention”, “fine chemical” or “the fine chemical” are used herein equally and relate in context of the paragraphs or sections [0014.1.13.13] to [515.1.1.13] essentially to the metabolite or the metabolites indicated in column 7, application no. 13 of Tables I, II and IV in the respective line.

Further, the term “in context of any of the paragraphs [0014.1.13.13] to [515.1.1.13]” as used herein means that for any of said paragraphs [0014.1.13.13] to [515.1.1.13] the term “the fine chemical” is understood to follow the definition of paragraphs or sections [0014.1.13.13] and [0015.1.13.13], independently whether it refers to any other paragraph or not and whether the reference recites the term “fine chemical” in an other context.

Thus, in cases where one or more paragraphs or sections are incorporated by reference into any of the present paragraphs [0014.1.13.13] to [515.1.1.13], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to the definition of paragraph [0014.1.13.13] and [0015.1.13.13].

Accordingly, in one embodiment, the term “fine chemical of the invention”, “fine chemical” or “the fine chemical” as used herein (alone or in combination, like in FCRP) means “abscisic acid in context of the nucleic acid or polypeptide sequences listed in the respective same line of any one of Tables I to IV of application no. 13 and indicating in column 7 the metabolite “abscisic acid”.

In one embodiment, the term abscisic acid or the term “fine chemical” mean in context of the paragraphs or sections [0014.1.13.13] to [515.1.1.13] at least one chemical compound with an activity of the above mentioned abscisic acid, respectively.

Accordingly, in one embodiment, the term “fine chemical of the invention”, “fine chemical” or “the fine chemical” as used herein (alone or in combination, like in FCRP) means “cryptoxanthin in context of the nucleic acid or polypeptide sequences listed in the respective same line of any one of Tables I to IV of application no. 13 and indicating in column 7 the metabolite “cryptoxanthin”.

In one embodiment, the term cryptoxanthin or the term “fine chemical” mean in context of the paragraphs or sections [0014.1.13.13] to [515.1.1.13] at least one chemical compound with an activity of the above mentioned cryptoxanthin, respectively.

Accordingly, in one embodiment, the term “fine chemical of the invention”, “fine chemical” or “the fine chemical” as used herein (alone or in combination, like in FCRP) means “violaxanthin in context of the nucleic acid or polypeptide sequences listed in the respective same line of any one of Tables I to IV of application no. 13 and indicating in column 7 the metabolite “violaxanthin”.

In one embodiment, the term violaxanthin or the term “fine chemical” mean in context of the paragraphs or sections [0014.1.13.13] to [515.1.1.13] at least one chemical compound with an activity of the above mentioned violaxanthin, respectively.

Accordingly, in one embodiment, the term “fine chemical of the invention”, “fine chemical” or “the fine chemical” as used herein (alone or in combination, like in FCRP) means “zeaxanthin in context of the nucleic acid or polypeptide sequences listed in the respective same line of any one of Tables I to IV of application no. 13 and indicating in column 7 the metabolite “zeaxanthin”.

In one embodiment, the term zeaxanthin or the term “fine chemical” mean in context of the paragraphs or sections [0014.1.13.13] to [515.1.1.13] at least one chemical compound with an activity of the above mentioned zeaxanthin, respectively.

Accordingly, in one embodiment, the terms “fine chemical of the invention”, “fine chemical” or “the fine chemical” mean in context of any of the paragraphs [0014.1.13.13] to [515.1.1.13] abscisic acid, its salts, ester or ether in free form or bound form. In a preferred embodiment, the term “the fine chemical” means abscisic acid or its salts, ester or ether, in free form or bound form. Other synonyms for abcisic acid are ABA, Dormin, [S-(Z,E)]-5-(1-Hydroxy-2,6,6-trimethyl-4-oxo-2-cyclohexen- 1-yl)-3-methyl-2,4-pentanedienoic acid, CAS-No 21293-29-8.

In a preferred embodiment “abscisic acid” means abscisic acid in free form. In a further embodiment “abscisic acid” means abscisic acid in form of sugar, preferably as abscisic acid glucose ester.

Accordingly, in one embodiment, the terms “fine chemical of the invention”, “fine chemical” or “the fine chemical” mean in context of any of the paragraphs [0014.1.13.13] to [515.1.1.13] cryptoxanthin, its salts, ester or ether in free form or bound form. In a preferred embodiment, the term “the fine chemical” means cryptoxanthin or its salts, ester or ether, in free form or bound form. Synonyms for cryptoxanthin are (R)-3,5,5-Trimethyl-4[3,7,12,16-tetramethyl-18-(2,6,6-trimethylcyclohex-1-enyl)-octadeca-1,3,5,7,9,11,13,15,17-nonaenyl]-cyclohex-3-enol, (3R)-β,β-Caroten-3-ol, Cryptoxanthol, Caricaxanthin.

In a preferred embodiment “cryptoxanthin” means cryptoxanthin in free form.

Accordingly, in one embodiment, the terms “fine chemical of the invention”, “fine chemical” or “the fine chemical” mean in context of any of the paragraphs [0014.1.13.13] to [515.1.1.13] violaxanthin, its salts, ester or ether in free form or bound form. In a preferred embodiment, the term “the fine chemical” means violaxanthin or its salts, ester or ether, in free form or bound form, preferably covalently bound (via ester-linkage) to fatty acids or in glycosylated form. Synonyms for violaxanthin are 1S,4S,6R)-1-[(1E,3E,5E,7E,9E,11E,13E,15E,17E)-18-[(1S,4S,6R)-4-Hydroxy-2,2,6-trimethyl-7-oxabicyclo[4.1.0]heptan-1-yl]-3,7,12,16-tetramethyloctadeca-1,3,5,7,9,11,13,15,17-nonaenyl]-2,2,6-trimethyl-7-oxabicyclo[4.1.0]heptan-4-ol,5,6:5′,6′-Diepoxy-5,5′,6,6′-tetrahydro-β-carotene-3,3′-diol, Zeaxanthin diepoxide, all-trans-Violaxanthin, E161e, CAS-No 126-29-4.

In a preferred embodiment “violaxanthin” means violaxanthin in free form.

Accordingly, in one embodiment, the terms “fine chemical of the invention”, “fine chemical” or “the fine chemical” mean in context of any of the paragraphs [0014.1.13.13] to [515.1.1.13] zeaxanthin, its salts, ester or ether in free form or bound form. In a preferred embodiment, the term “the fine chemical” means zeaxanthin or its salts, ester or ether, in free form or bound form, preferably to a glucoside, e.g a diglucoside. Other names for zeaxanthin are for example 4-[18-(4-hydroxy-2,6,6-trimethyl-1-cyclohexenyl)-3,7,12,16-tetramethyl-octadeca-1,3,5,7,9,11,13,15,17-nonaenyl]-3,5,5-trimethyl-cyclohex-3-en-1-ol, β,β-carotene-3,3′-diol.

In a preferred embodiment “zeaxanthin” means zeaxanthin in free form.

In one embodiment, the term “xanthophyll” or “the fine chemical” or “the respective fine chemical” mean in context of any of the paragraphs [0014.1.13.13] to [515.1.1.13] at least one chemical compound with xanthophyll activity selected from the group “abscisic acid, cryptoxanthin, violaxanthin, and zeaxanthin”.

Further, the term “in context of any of the paragraphs [0014.1.13.13] to [515.1.1.13]” as used herein means that for any of said paragraphs [0014.1.13.13] to [515.1.1.13] the term “the fine chemical” is understood to follow the definition of section [0014.1.13.13] or section [independently whether it refers to any other paragraph or not and whether the reference recites the term “fine chemical” in an other context.

Thus, in cases where one or more paragraphs or sections are incorporated by reference into any of the present paragraphs [0014.1.13.13] to [515.1.1.13], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to this definition of this paragraph [0015.1.13.13].

Further, the term “fine chemicals” as used herein relates to compositions comprising said fine chemical(s), i.e. comprising abscisic acid, cryptoxanthin, violaxanthin, and/or zeaxanthin, respectively.

Accordingly, the present invention relates to a process for the production of cryptoxanthin, which comprises
(a) increasing or generating one or more activities selected from the group consisting of thioredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the pro-duction of cryptoxanthin or a composition comprising cryptoxanthin in said non-human organism or in the culture medium surrounding said non-human organism.

Accordingly, the present invention relates to a process for the production of zeaxanthin, which comprises
(a) increasing or generating one or more activities selected from the group consisting of F-box protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of zeaxanthin, which comprises
(a) increasing or generating one or more activities selected from the group consisting of monthiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of zeaxanthin, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of zeaxanthin, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of zeaxanthin, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of cryptoxanthin, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of zeaxanthin, which comprises
(a) increasing or generating one or more activities selected from the group consisting of MADS-box transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of zeaxanthin, which comprises
(a) increasing or generating one or more activities selected from the group consisting of isopropylmalate isomerase large subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of cryptoxanthin, which comprises
(a) increasing or generating one or more activities selected from the group consisting of AX653549-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of zeaxanthin, which comprises
(a) increasing or generating one or more activities selected from the group consisting of b1003-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of zeaxanthin, which comprises
(a) increasing or generating one or more activities selected from the group consisting of lipoprotein precursor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of zeaxanthin, which comprises
(a) increasing or generating one or more activities selected from the group consisting of serine/threonine-protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of zeaxanthin, which comprises
(a) increasing or generating one or more activities selected from the group consisting of b2032-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of cryptoxanthin, which comprises
(a) increasing or generating one or more activities selected from the group consisting of b2099-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of zeaxanthin, which comprises
(a) increasing or generating one or more activities selected from the group consisting of b2121-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of zeaxanthin, which comprises
(a) increasing or generating one or more activities selected from the group consisting of b2474-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of zeaxanthin, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of cryptoxanthin, which comprises
(a) increasing or generating one or more activities selected from the group consisting of flavodoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of violaxanthin, which comprises
(a) increasing or generating one or more activities selected from the group consisting of ribosephosphate isomerase, constitutive in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of violaxanthin or a composition comprising violaxanthin in said non-human organism or in the culture medium surrounding said non-human organism.

Accordingly, the present invention relates to a process for the production of violaxanthin, which comprises
(a) increasing or generating one or more activities selected from the group consisting of acetyl CoA carboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of violaxanthin or a composition comprising violaxanthin in said non-human organism or in the culture medium surrounding said non-human organism.

Accordingly, the present invention relates to a process for the production of cryptoxanthin, which comprises
(a) increasing or generating one or more activities selected from the group consisting of b3346-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of violaxanthin, which comprises
(a) increasing or generating one or more activities selected from the group consisting of ketol-acid reductoisomerase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of violaxanthin or a composition comprising violaxanthin in said non-human organism or in the culture medium surrounding said non-human organism.

Accordingly, the present invention relates to a process for the production of cryptoxanthin, which comprises
(a) increasing or generating one or more activities selected from the group consisting of b3817-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of violaxanthin, which comprises
(a) increasing or generating one or more activities selected from the group consisting of superoxide dismutase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of violaxanthin or a composition comprising violaxanthin in said non-human organism or in the culture medium surrounding said non-human organism.

Accordingly, the present invention relates to a process for the production of violaxanthin, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of violaxanthin or a composition comprising violaxanthin in said non-human organism or in the culture medium surrounding said non-human organism.

Accordingly, the present invention relates to a process for the production of cryptoxanthin, which comprises
(a) increasing or generating one or more activities selected from the group consisting of melibiose carrier protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of zeaxanthin, which comprises
(a) increasing or generating one or more activities selected from the group consisting of melibiose carrier protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the pro-duction of zeaxanthin or a composition comprising zeaxanthin in said non-human organism or in the culture medium surrounding said non-human organism.

Accordingly, the present invention relates to a process for the production of violaxanthin, which comprises
(a) increasing or generating one or more activities selected from the group consisting of purine-nucleoside phosphorylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of violaxanthin or a composition comprising violaxanthin in said non-human organism or in the culture medium surrounding said non-human organism.

Accordingly, the present invention relates to a process for the production of zeaxanthin, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of cryptoxanthin, which comprises
(a) increasing or generating one or more activities selected from the group consisting of coproporphyrinogen oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of zeaxanthin, which comprises
(a) increasing or generating one or more activities selected from the group consisting of coproporphyrinogen oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of zeaxanthin, which comprises
(a) increasing or generating one or more activities selected from the group consisting of glycerol-3-phosphate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of cryptoxanthin, which comprises
(a) increasing or generating one or more activities selected from the group consisting of YCL026C-A-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of cryptoxanthin, which comprises
(a) increasing or generating one or more activities selected from the group consisting of F-box protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of cryptoxanthin, which comprises
(a) increasing or generating one or more activities selected from the group consisting of ygl096w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of cryptoxanthin, which comprises
(a) increasing or generating one or more activities selected from the group consisting of alcohol dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of cryptoxanthin, which comprises
(a) increasing or generating one or more activities selected from the group consisting of halotolerance protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of zeaxanthin, which comprises
(a) increasing or generating one or more activities selected from the group consisting of halotolerance protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of zeaxanthin, which comprises
(a) increasing or generating one or more activities selected from the group consisting of N-acetylglucosaminylphosphatidylinositoldeacetylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of cryptoxanthin, which comprises
(a) increasing or generating one or more activities selected from the group consisting of ymr324c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of zeaxanthin, which comprises
(a) increasing or generating one or more activities selected from the group consisting of sterol O-acyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of abscisic acid, which comprises
(a) increasing or generating one or more activities selected from the group consisting of YER184C-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

Accordingly, the present invention relates to a process for the production of abscisic acid, which comprises
(a) increasing or generating one or more activities selected from the group consisting of YOR044W-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

Accordingly, the present invention relates to a process for the production of abscisic acid, which comprises
(a) increasing or generating one or more activities selected from the group consisting of gluconate transport system permease 3 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

Accordingly, the present invention relates to a process for the production of abscisic acid, which comprises
(a) increasing or generating one or more activities selected from the group consisting of nitrate/nitrite transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

Accordingly, the present invention relates to a process for the production of abscisic acid, which comprises
(a) increasing or generating one or more activities selected from the group consisting of acyl transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

Accordingly, the present invention relates to a process for the production of abscisic acid, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

Accordingly, the present invention relates to a process for the production of abscisic acid, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

Accordingly, the present invention relates to a process for the production of abscisic acid, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

Accordingly, the present invention relates to a process for the production of abscisic acid, which comprises
(a) increasing or generating one or more activities selected from the group consisting of glycine cleavage system T aminomethyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

Accordingly, the present invention relates to a process for the production of abscisic acid, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

An embodiment of the present invention relates to a process for the production of abscisic acid, which comprises
(a1) increasing or generating the activity of a polypeptide comprising a polypeptide as depicted in a respective line in column 5 or 8 of Table II, application no. 13, or a homolog or a fragment thereof, a consensus sequence or at least one polypeptide motif as depicted in the respective line in column 8 of Table IV, application no. 13, whereby the respective line disclose in column 7 the fine chemical abscisic acid; or
(a2) increasing or generating the activity of an expression product of one or more nucleic acid molecule(s) comprising a polynucleotide as depicted in the respective line in column 5 or 8 of Table I, application no. 13, preferably the coding region thereof, or a homolog or a fragment thereof, whereby the respective line disclose in column 7 the fine chemical abscisic acid; or
(a3) increasing or generating the activity of a functional equivalent of (a1) or (a2); in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of abscisic acid or a composition comprising abscisic acid in said non-human organism or in the culture medium surrounding said non-human organism.

An embodiment of the present invention relates to a process for the production of cryptoxanthin, which comprises
(a1) increasing or generating the activity of a polypeptide comprising a polypeptide as depicted in a respective line in column 5 or 8 of Table II, application no. 13, or a homolog or a fragment thereof, a consensus sequence or at least one polypeptide motif as depicted in the respective line in column 8 of Table IV, application no. 13, whereby the respective line disclose in column 7 the fine chemical cryptoxanthin; or
(a2) increasing or generating the activity of an expression product of one or more nucleic acid molecule(s) comprising a polynucleotide as depicted in the respective line in column 5 or 8 of Table I, application no. 13, preferably the coding region thereof, or a homolog or a fragment thereof, whereby the respective line disclose in column 7 the fine chemical cryptoxanthin; or
(a3) increasing or generating the activity of a functional equivalent of (a1) or (a2); in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of cryptoxanthin or a composition comprising cryptoxanthin in said non-human organism or in the culture medium surrounding said non-human organism.

An embodiment of the present invention relates to a process for the production of violaxanthin, which comprises
(a1) increasing or generating the activity of a polypeptide comprising a polypeptide as depicted in a respective line in column 5 or 8 of Table II, application no. 13, or a homolog or a fragment thereof, a consensus sequence or at least one polypeptide motif as depicted in the respective line in column 8 of Table IV, application no. 13, whereby the respective line disclose in column 7 the fine chemical violaxanthin; or
(a2) increasing or generating the activity of an expression product of one or more nucleic acid molecule(s) comprising a polynucleotide as depicted in the respective line in column 5 or 8 of Table I, application no. 13, preferably the coding region thereof, or a homolog or a fragment thereof, whereby the respective line disclose in column 7 the fine chemical violaxanthin; or
(a3) increasing or generating the activity of a functional equivalent of (a1) or (a2); in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of violaxanthin or a composition comprising violaxanthin in said non-human organism or in the culture medium surrounding said non-human organism.

An embodiment of the present invention relates to a process for the production of zeaxanthin, which comprises
(a1) increasing or generating the activity of a polypeptide comprising a polypeptide as depicted in a respective line in column 5 or 8 of Table II, application no. 13, or a homolog or a fragment thereof, a consensus sequence or at least one polypeptide motif as depicted in the respective line in column 8 of Table IV, application no. 13, whereby the respective line disclose in column 7 the fine chemical zeaxanthin; or
(a2) increasing or generating the activity of an expression product of one or more nucleic acid molecule(s) comprising a polynucleotide as depicted in the respective line in column 5 or 8 of Table I, application no. 13, preferably the coding region thereof, or a homolog or a fragment thereof, whereby the respective line disclose in column 7 the fine chemical zeaxanthin; or
(a3) increasing or generating the activity of a functional equivalent of (a1) or (a2); in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of zeaxanthin or a composition comprising zeaxanthin in said non-human organism or in the culture medium surrounding said non-human organism.

Accordingly, the increase or generation of one or more said activities is for example conferred by one or more expression products of said nucleic acid molecule, e.g. proteins. Accordingly, in the present invention described above, the increase or generation of one or more said activities is for example conferred by one or more protein(s) each comprising a polypeptide selected from the group as depicted in Table II, application no. 13, column 5 and 8, or a homolog or a fragment thereof.

The process of the invention comprises in one embodiment the following steps:
(i) increasing or generating of the expression of; and/or
(ii) increasing or generating the expression of an expression product of; and/or
(iii) increasing or generating one or more activities of an expression product encoded by; at least one nucleic acid molecule (in the following “Fine Chemical Related Protein (FCRP)”-encoding gene or “FCRP”-gene) comprising a nucleic acid molecule selected from the group consisting of:
(a) a nucleic acid molecule encoding the polypeptide shown in column 5 or 8 of Table II, preferably Table II B, application no. 13, or a homolog or a fragment thereof;
(b) a nucleic acid molecule shown in column 5 or 8 of Table I, preferably Table I B, application no. 13, or a homolog or a fragment thereof (preferably the coding region thereof);
(c) a nucleic acid molecule, which, as a result of the degeneracy of the genetic code, can be derived from a polypeptide sequence depicted in column 5 or 8 of Table II, preferably Table II B, application no. 13;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 13, or the coding region thereof;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 13;
(i) a nucleic acid molecule encoding a polypeptide having the activity represented by a respective protein as depicted in column 5 of Table II, application no. 13;
(j) a nucleic acid molecule which comprises a polynucleotide, which is obtained by amplifying a cDNA library or a genomic library using the primers as depicted in column 8 of Table III, application no. 13; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment thereof said nucleic acid molecule encodes a polypeptide which has the activity of the polypeptide represented by a protein comprising a polypeptide as depicted in the corresponding hit in column 5 of Table II, application no. 13.

In another preferred embodiment thereof said nucleic acid molecule confers a generation or an increase of the respective fine chemical in a non-human organism or a part thereof as compared to a corresponding wild-type non-human organism or part thereof.

In another preferred embodiment thereof said nucleic acid molecule encodes a polypeptide which has the activity of the polypeptide represented by a protein as depicted in the corresponding hit in column 5 of Table II, application no. 13, and confers a generation or an increase of the respective fine chemical in a non-human organism or a part thereof as compared to a corresponding wild-type non-human organism or part thereof.

Accordingly, the genes of the present invention or used in accordance with the present invention, which respectively encode a protein having an activity of acetyl CoA carboxylase, acetyltransferase, acyl transferase, alcohol dehydrogenase, AX653549-protein, b1003-protein, b2032-protein, b2099-protein, b2121-protein, b2474-protein, b3346-protein, b3817-protein, coproporphyrinogen oxidase, F-box protein, flavodoxin, gluconate transport system permease 3, glutaredoxin, glycerol-3-phosphate dehydrogenase, glycine cleavage system T aminomethyltransferase, halotolerance protein, heat shock transcription factor, isopropylmalate isomerase large subunit, ketol-acid reductoisomerase, lipoprotein precursor, MADS-box transcription factor, melibiose carrier protein, monothiol glutaredoxin, monthiol glutaredoxin, N-acetylglucosaminylphosphatidylinositoldeacetylase, nitrate/nitrite transport protein, protein kinase, protein phosphatase, purine-nucleoside phosphorylase, ribosephosphate isomerase, constitutive, serine/threonine-protein phosphatase, sterol O-acyltransferase, superoxide dismutase, thioredoxin, transcription factor, transcriptional regulator, YCL026C-A-protein, YER184C-protein, ygl096w-protein, ymr324c-protein, or YOR044W-protein, which respectively encode a protein comprising a polypeptide encoded by a nucleic acid sequence as shown in Table I, application no. 13, column 5 or 8, (preferably the coding region thereof), or a homolog or a fragment thereof, which respectively encode a protein comprising a polypeptide as depicted in Table II, application no. 13, column 5 or 8, or a homolg or a fragment thereof, and/or which respectively can be amplified with the primer set shown in Table III, application no. 13, column 8, are also referred to as “FCRP genes”.

Proteins or polypeptides encoded by “FCRP-genes” are referred to as “Fine Chemical Related Proteins” or “FCRP”. For the purposes of the description of the present invention, the respective protein having an activity of acetyl CoA carboxylase, acetyltransferase, acyl transferase, alcohol dehydrogenase, AX653549-protein, b1003-protein, b2032-protein, b2099-protein, b2121-protein, b2474-protein, b3346-protein, b3817-protein, coproporphyrinogen oxidase, F-box protein, flavodoxin, gluconate transport system permease 3, glutaredoxin, glycerol-3-phosphate dehydrogenase, glycine cleavage system T aminomethyltransferase, halotolerance protein, heat shock transcription factor, isopropylmalate isomerase large subunit, ketolacid reductoisomerase, lipoprotein precursor, MADS-box transcription factor, melibiose carrier protein, monothiol glutaredoxin, monthiol glutaredoxin, N-acetylglucosaminylphosphatidylinositoldeacetylase, nitrate/nitrite transport protein, protein kinase, protein phosphatase, purine-nucleoside phosphorylase, ribosephosphate isomerase, constitutive, serine/threonine-protein phosphatase, sterol O-acyltransferase, superoxide dismutase, thioredoxin, transcription factor, transcriptional regulator, YCL026C-A-protein, YER184C-protein, ygl096w-protein, ymr324c-protein, or YOR044W-protein, the respective protein comprising a polypeptide encoded by one or more respective nucleic acid sequences as shown in Table I, application no. 13, column 5 or 8, (preferably the coding region thereof), or a homolog or fragment thereof, the respective protein comprising a respective polypeptide as depicted in Table II, application no. 13, column 5 or 8, or a homolog or fragment thereof, the respective protein comprising a sequence corresponding to the consensus sequence as shown in Table IV, application no. 13, column 8, and/or the respective protein comprising at least one polypeptide motif as shown in Table IV, application no. 13, column 8 is also referred to as Fine Chemical Related Protein” or “FCRP”.

Thus, in one embodiment, the present invention provides a process of the production of abscisic acid, cryptoxanthin, violaxanthin, and/or zeaxanthin, by increasing or generating one or more activities, especially selected from the group consisting of acetyl CoA carboxylase, acetyltransferase, acyl transferase, alcohol dehydrogenase, AX653549-protein, b1003-protein, b2032-protein, b2099-protein, b2121-protein, b2474-protein, b3346-protein, b3817-protein, coproporphyrinogen oxidase, F-box protein, flavodoxin, gluconate transport system permease 3, glutaredoxin, glycerol-3-phosphate dehydrogenase, glycine cleavage system T aminomethyltransferase, halotolerance protein, heat shock transcription factor, isopropylmalate isomerase large subunit, ketol-acid reductoisomerase, lipoprotein precursor, MADS-transcription factor, melibiose carrier protein, monothiol glutaredoxin, monthiol glutaredoxin, N-acetylglucosaminylphosphatidylinositoldeacetylase, nitrate/nitrite transport protein, protein kinase, protein phosphatase, purine-nucleoside phosphorylase, ribosephosphate isomerase, constitutive, serine/threonine-protein phosphatase, sterol O-acyltransferase, superoxide dismutase, thioredoxin, transcription factor, transcriptional regulator, YCL026C-A-protein, YER184C-protein, ygl096w-protein, ymr324c-protein, and YOR044W-protein, which is conferred by one or more FCRPs or the gene product of one or more FCRP-genes, for example by the gene product of a nucleic acid sequences comprising a polynucleotide selected from the group as shown in Table I, application no. 13, column 5 or 8, (preferably by the coding region thereof), or a homolog or a fragment thereof, e.g. or by one or more proteins each comprising a polypeptide encoded by one or more nucleic acid sequences selected from the group as shown in Table I, application no. 13, column 5 or 8, (preferably by the coding region thereof), or a homolog or a fragment thereof, or by one or more protein(s) each comprising a polypeptide selected from the group as depicted in Table II, application no. 13, column 5 and 8, or a homolog thereof, or a protein comprising a sequence corresponding to the consensus sequence or comprising at least one polypeptide motif as shown in Table IV, application no. 13, column 8.

for the disclosure of this paragraph see [0025.1.7.7] above

In an embodiment, the process comprises increasing or generating the activity of one or more polypeptides having said activity, e.g. by generating or increasing the amount and/or specific activity in the cell or a compartment of a cell of one of more FCRP, especially selected from the group consisting of acetyl CoA carboxylase, acetyltransferase, acyl transferase, alcohol dehydrogenase, AX653549-protein, b1003-protein, b2032-protein, b2099-protein, b2121-protein, b2474-protein, b3346-protein, b3817-protein, coproporphyrinogen oxidase, F-box protein, flavodoxin, gluconate transport system permease 3, glutaredoxin, glycerol-3-phosphate dehydrogenase, glycine cleavage system T aminomethyltransferase, halotolerance protein, heat shock transcription factor, isopropylmalate isomerase large subunit, ketol-acid reductoisomerase, lipoprotein precursor, MADS-box transcription factor, melibiose carrier protein, monothiol glutaredoxin, monthiol glutaredoxin, N-acetylglucosaminylphosphatidylinositoldeacetylase, nitrate/nitrite transport protein, protein kinase, protein phosphatase, purine-nucleoside phosphorylase, ribosephosphate isomerase, constitutive, serine/threonine-protein phosphatase, sterol O-acyltransferase, superoxide dismutase, thioredoxin, transcription factor, transcriptional regulator, YCL026C-A-protein, YER184C-protein, ygl096w-protein, ymr324c-protein, and YOR044W-protein, for example of the respective polypeptide as depicted in Table II, application no. 13, column 5 and 8, or a homolog or a fragment thereof, or the respective polypeptide comprising a sequence corresponding to the consensus sequences as shown in Table IV, application no. 13, column 8, or the respective polypeptide comprising at least one polypeptide motif as depicted in Table IV, application no. 13, column 8.

A further embodiment of the present invention relates to a process for the production of cryptoxanthin, which comprises
(a) increasing or generating the activity of a thioredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of zeaxanthin, which comprises
(a) increasing or generating the activity of a F-box protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of zeaxanthin, which comprises
(a) increasing or generating the activity of a monthiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of zeaxanthin, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of zeaxanthin, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of zeaxanthin, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of cryptoxanthin, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of zeaxanthin, which comprises
(a) increasing or generating the activity of a MADS-box transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of zeaxanthin, which comprises
(a) increasing or generating the activity of a isopropylmalate isomerase large subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of cryptoxanthin, which comprises
(a) increasing or generating the activity of a AX653549-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of zeaxanthin, which comprises
(a) increasing or generating the activity of a b1003-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of zeaxanthin, which comprises
(a) increasing or generating the activity of a lipoprotein precursor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of zeaxanthin, which comprises
(a) increasing or generating the activity of a serine/threonine-protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of zeaxanthin, which comprises
(a) increasing or generating the activity of a b2032-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of cryptoxanthin, which comprises
(a) increasing or generating the activity of a b2099-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of zeaxanthin, which comprises
(a) increasing or generating the activity of a b2121-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of zeaxanthin, which comprises
(a) increasing or generating the activity of a b2474-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of zeaxanthin, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of cryptoxanthin, which comprises
(a) increasing or generating the activity of a flavodoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of violaxanthin, which comprises
(a) increasing or generating the activity of a ribosephosphate isomerase, constitutive non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of violaxanthin or a composition comprising violaxanthin in said non-human organism or in the culture medium surrounding said non-human organism.

A further embodiment of the present invention relates to a process for the production of violaxanthin, which comprises
(a) increasing or generating the activity of a acetyl CoA carboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of violaxanthin or a composition comprising violaxanthin in said non-human organism or in the culture medium surrounding said non-human organism.

A further embodiment of the present invention relates to a process for the production of cryptoxanthin, which comprises
(a) increasing or generating the activity of a b3346-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of violaxanthin, which comprises
(a) increasing or generating the activity of a ketol-acid reductoisomerase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of violaxanthin or a composition comprising violaxanthin in said non-human organism or in the culture medium surrounding said non-human organism.

A further embodiment of the present invention relates to a process for the production of cryptoxanthin, which comprises
(a) increasing or generating the activity of a b3817-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of violaxanthin, which comprises
(a) increasing or generating the activity of a superoxide dismutase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of violaxanthin or a composition comprising violaxanthin in said non-human organism or in the culture medium surrounding said non-human organism.

A further embodiment of the present invention relates to a process for the production of violaxanthin, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of violaxanthin or a composition comprising violaxanthin in said non-human organism or in the culture medium surrounding said non-human organism.

A further embodiment of the present invention relates to a process for the production of cryptoxanthin, which comprises
(a) increasing or generating the activity of a melibiose carrier protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of zeaxanthin, which comprises
(a) increasing or generating the activity of a melibiose carrier protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of violaxanthin, which comprises
(a) increasing or generating the activity of a purine-nucleoside phosphorylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of violaxanthin or a composition comprising violaxanthin in said non-human organism or in the culture medium surrounding said non-human organism.

A further embodiment of the present invention relates to a process for the production of zeaxanthin, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of cryptoxanthin, which comprises
(a) increasing or generating the activity of a coproporphyrinogen oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of zeaxanthin, which comprises
(a) increasing or generating the activity of a coproporphyrinogen oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of zeaxanthin, which comprises
(a) increasing or generating the activity of a glycerol-3-phosphate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of cryptoxanthin, which comprises
(a) increasing or generating the activity of a YCL026C-A-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of cryptoxanthin, which comprises
(a) increasing or generating the activity of a F-box protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of cryptoxanthin, which comprises
(a) increasing or generating the activity of a ygl096w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of cryptoxanthin, which comprises
(a) increasing or generating the activity of a alcohol dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of cryptoxanthin, which comprises
(a) increasing or generating the activity of a halotolerance protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of zeaxanthin, which comprises
(a) increasing or generating the activity of a halotolerance protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of zeaxanthin, which comprises
(a) increasing or generating the activity of a N-acetylglucosaminylphosphatidylinositoldeacetylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of cryptoxanthin, which comprises
(a) increasing or generating the activity of a ymr324c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of zeaxanthin, which comprises
(a) increasing or generating the activity of a sterol O-acyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of abscisic acid, which comprises
(a) increasing or generating the activity of a YER184C-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

A further embodiment of the present invention relates to a process for the production of abscisic acid, which comprises
(a) increasing or generating the activity of a YOR044W-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

A further embodiment of the present invention relates to a process for the production of abscisic acid, which comprises
(a) increasing or generating the activity of a gluconate transport system permease 3 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

A further embodiment of the present invention relates to a process for the production of abscisic acid, which comprises
(a) increasing or generating the activity of a nitrate/nitrite transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

A further embodiment of the present invention relates to a process for the production of abscisic acid, which comprises
(a) increasing or generating the activity of a acyl transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

A further embodiment of the present invention relates to a process for the production of abscisic acid, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

A further embodiment of the present invention relates to a process for the production of abscisic acid, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

A further embodiment of the present invention relates to a process for the production of abscisic acid, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

A further embodiment of the present invention relates to a process for the production of abscisic acid, which comprises
(a) increasing or generating the activity of a glycine cleavage system T aminomethyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

A further embodiment of the present invention relates to a process for the production of abscisic acid, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

A further embodiment of the present invention relates to a process for the production of abscisic acid, which comprises
(a) increasing or generating the activity of a polypeptide comprising a polypeptide as depicted in the respective line in column 5 or 8 of Table II, application no. 13, or a homolog or a fragment thereof, a consensus sequence or at least one polypeptide motif as depicted in the respective line in column 8 of Table IV, application no. 13, whereby the respective line disclose in column 7 the fine chemical abscisic acid; or
increasing or generating the activity of an expression product of one or more nucleic acid molecule(s) comprising a polynucleotide as depicted in the respective line in column 5 or 8 of Table I, application no. 13, preferably the coding region thereof, or a homolog or a fragment thereof, whereby the respective line disclose in column 7 the fine chemical abscisic acid;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of abscisic acid, or a composition comprising abscisic acid in said non-human organism or in the culture medium surrounding said non-human organism.

A further embodiment of the present invention relates to a process for the production of cryptoxanthin, which comprises
(a) increasing or generating the activity of a polypeptide comprising a polypeptide as depicted in the respective line in column 5 or 8 of Table II, application no. 13, or a homolog or a fragment thereof, a consensus sequence or at least one polypeptide motif as depicted in the respective line in column 8 of Table IV, application no. 13, whereby the respective line disclose in column 7 the fine chemical cryptoxanthin; or
increasing or generating the activity of an expression product of one or more nucleic acid molecule(s) comprising a polynucleotide as depicted in the respective line in column 5 or 8 of Table I, application no. 13, preferably the coding region thereof, or a homolog or a fragment thereof, whereby the respective line disclose in column 7 the fine chemical cryptoxanthin;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of cryptoxanthin, or a composition comprising cryptoxanthin in said non-human organism or in the culture medium surrounding said non-human organism.

A further embodiment of the present invention relates to a process for the production of violaxanthin, which comprises
(a) increasing or generating the activity of a polypeptide comprising a polypeptide as depicted in the respective line in column 5 or 8 of Table II, application no. 13, or a homolog or a fragment thereof, a consensus sequence or at least one polypeptide motif as depicted in the respective line in column 8 of Table IV, application no. 13, whereby the respective line disclose in column 7 the fine chemical violaxanthin; or
increasing or generating the activity of an expression product of one or more nucleic acid molecule(s) comprising a polynucleotide as depicted in the respective line in column 5 or 8 of Table I, application no. 13, preferably the coding region thereof, or a homolog or a fragment thereof, whereby the respective line disclose in column 7 the fine chemical violaxanthin;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of violaxanthin, or a composition comprising violaxanthin in said non-human organism or in the culture medium surrounding said non-human organism.

A further embodiment of the present invention relates to a process for the production of zeaxanthin, which comprises
(a) increasing or generating the activity of a polypeptide comprising a polypeptide as depicted in the respective line in column 5 or 8 of Table II, application no. 13, or a homolog or a fragment thereof, a consensus sequence or at least one polypeptide motif as depicted in the respective line in column 8 of Table IV, application no. 13, whereby the respective line disclose in column 7 the fine chemical zeaxanthin; or
increasing or generating the activity of an expression product of one or more nucleic acid molecule(s) comprising a polynucleotide as depicted in the respective line in column 5 or 8 of Table I, application no. 13, preferably the coding region thereof, or a homolog or a fragment thereof, whereby the respective line disclose in column 7 the fine chemical zeaxanthin;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of zeaxanthin, or a composition comprising zeaxanthin in said non-human organism or in the culture medium surrounding said non-human organism.

A further embodiment of the present invention relates to a process for the production of cryptoxanthin, which comprises
(a) increasing or generating one or more activities selected from the group consisting of thioredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of zeaxanthin, which comprises
(a) increasing or generating one or more activities selected from the group consisting of F-box protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of zeaxanthin, which comprises
(a) increasing or generating one or more activities selected from the group consisting of monthiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of zeaxanthin, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of zeaxanthin, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of zeaxanthin, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of cryptoxanthin, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of zeaxanthin, which comprises
(a) increasing or generating one or more activities selected from the group consisting of MADS-box transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of zeaxanthin, which comprises
(a) increasing or generating one or more activities selected from the group consisting of isopropylmalate isomerase large subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of cryptoxanthin, which comprises
(a) increasing or generating one or more activities selected from the group consisting of AX653549-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of zeaxanthin, which comprises
(a) increasing or generating one or more activities selected from the group consisting of b1003-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of zeaxanthin, which comprises
(a) increasing or generating one or more activities selected from the group consisting of lipoprotein precursor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of zeaxanthin, which comprises
(a) increasing or generating one or more activities selected from the group consisting of serine/threonine-protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of zeaxanthin, which comprises
(a) increasing or generating one or more activities selected from the group consisting of b2032-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of cryptoxanthin, which comprises
(a) increasing or generating one or more activities selected from the group consisting of b2099-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of zeaxanthin, which comprises
(a) increasing or generating one or more activities selected from the group consisting of b2121-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of zeaxanthin, which comprises
(a) increasing or generating one or more activities selected from the group consisting of b2474-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of zeaxanthin, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of cryptoxanthin, which comprises
(a) increasing or generating one or more activities selected from the group consisting of flavodoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of violaxanthin, which comprises
(a) increasing or generating one or more activities selected from the group consisting of ribosephosphate isomerase, constitutive in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of violaxanthin or a composition comprising violaxanthin in said non-human organism or in the culture medium surrounding said non-human organism.

A further embodiment of the present invention relates to a process for the production of violaxanthin, which comprises
(a) increasing or generating one or more activities selected from the group consisting of acetyl CoA carboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of violaxanthin or a composition comprising violaxanthin in said non-human organism or in the culture medium surrounding said non-human organism.

A further embodiment of the present invention relates to a process for the production of cryptoxanthin, which comprises
(a) increasing or generating one or more activities selected from the group consisting of b3346-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of violaxanthin, which comprises
(a) increasing or generating one or more activities selected from the group consisting of ketol-acid reductoisomerase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of violaxanthin or a composition comprising violaxanthin in said non-human organism or in the culture medium surrounding said non-human organism.

A further embodiment of the present invention relates to a process for the production of cryptoxanthin, which comprises
(a) increasing or generating one or more activities selected from the group consisting of b3817-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of violaxanthin, which comprises
(a) increasing or generating one or more activities selected from the group consisting of superoxide dismutase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of violaxanthin or a composition comprising violaxanthin in said non-human organism or in the culture medium surrounding said non-human organism.

A further embodiment of the present invention relates to a process for the production of violaxanthin, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of violaxanthin or a composition comprising violaxanthin in said non-human organism or in the culture medium surrounding said non-human organism.

A further embodiment of the present invention relates to a process for the production of cryptoxanthin, which comprises
(a) increasing or generating one or more activities selected from the group consisting of melibiose carrier protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of zeaxanthin, which comprises
(a) increasing or generating one or more activities selected from the group consisting of melibiose carrier protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of violaxanthin, which comprises
(a) increasing or generating one or more activities selected from the group consisting of purine-nucleoside phosphorylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of violaxanthin or a composition comprising violaxanthin in said non-human organism or in the culture medium surrounding said non-human organism.

A further embodiment of the present invention relates to a process for the production of zeaxanthin, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of cryptoxanthin, which comprises
(a) increasing or generating one or more activities selected from the group consisting of coproporphyrinogen oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of zeaxanthin, which comprises
(a) increasing or generating one or more activities selected from the group consisting of coproporphyrinogen oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of zeaxanthin, which comprises
(a) increasing or generating one or more activities selected from the group consisting of glycerol-3-phosphate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of cryptoxanthin, which comprises
(a) increasing or generating one or more activities selected from the group consisting of YCL026C-A-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of cryptoxanthin, which comprises
(a) increasing or generating one or more activities selected from the group consisting of F-box protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of cryptoxanthin, which comprises
(a) increasing or generating one or more activities selected from the group consisting of ygl096w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of cryptoxanthin, which comprises
(a) increasing or generating one or more activities selected from the group consisting of alcohol dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of cryptoxanthin, which comprises
(a) increasing or generating one or more activities selected from the group consisting of halotolerance protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of zeaxanthin, which comprises
(a) increasing or generating one or more activities selected from the group consisting of halotolerance protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of zeaxanthin, which comprises
(a) increasing or generating one or more activities selected from the group consisting of N-acetylglucosaminylphosphatidylinositoldeacetylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of cryptoxanthin, which comprises
(a) increasing or generating one or more activities selected from the group consisting of ymr324c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

A further embodiment of the present invention relates to a process for the production of zeaxanthin, which comprises
(a) increasing or generating one or more activities selected from the group consisting of sterol O-acyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

A further embodiment of the present invention relates to a process for the production of abscisic acid, which comprises
(a) increasing or generating one or more activities selected from the group consisting of YER184C-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

A further embodiment of the present invention relates to a process for the production of abscisic acid, which comprises
(a) increasing or generating one or more activities selected from the group consisting of YOR044W-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

A further embodiment of the present invention relates to a process for the production of abscisic acid, which comprises
(a) increasing or generating one or more activities selected from the group consisting of gluconate transport system permease 3 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

A further embodiment of the present invention relates to a process for the production of abscisic acid, which comprises
(a) increasing or generating one or more activities selected from the group consisting of nitrate/nitrite transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

A further embodiment of the present invention relates to a process for the production of abscisic acid, which comprises
(a) increasing or generating one or more activities selected from the group consisting of acyl transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

A further embodiment of the present invention relates to a process for the production of abscisic acid, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

A further embodiment of the present invention relates to a process for the production of abscisic acid, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

A further embodiment of the present invention relates to a process for the production of abscisic acid, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

A further embodiment of the present invention relates to a process for the production of abscisic acid, which comprises
(a) increasing or generating one or more activities selected from the group consisting of glycine cleavage system T aminomethyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

A further embodiment of the present invention relates to a process for the production of abscisic acid, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

A further embodiment of the present invention relates to a process for the production of abscisic acid, which comprises
(a1) increasing or generating the activity of a polypeptide comprising a polypeptide as depicted in the respective line in column 5 or 8 of Table II, application no. 13, or a homolog or fragment thereof, a consensus sequence or at least one polypeptide motif as depicted in column 8 of Table IV, application no. 13, whereby the respective line disclose in column 7 the fine chemical abscisic acid; or
increasing or generating the activity of an expression product of one or more nucleic acid molecule(s) comprising a polynucleotide as depicted in the respective line in column 5 or 8 of Table I, application no. 13, preferably the coding region thereof, or a homolog or a fragment thereof, whereby the respective line disclose in column 7 the fine chemical abscisic acid;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 13, or a homolog or a fragment thereof, a consensus sequence or at least one polypeptide motif as depicted in the respective line in column 8 of Table IV, application no. 13, which is joined to a transit peptide, whereby the respective line disclose in column 7 the fine chemical abscisic acid; or
increasing or generating the activity of an expression product of one or more nucleic acid molecule(s) comprising a polynucleotide as depicted in the respective line in column 5 or 8 of Table I, application no. 13, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical abscisic acid;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 13, or a homolog or a fragment thereof, a consensus sequence or at least one polypeptide motif as depicted in the respective line in column 8 of Table IV, application no. 13, whereby the respective line disclose in column 7 the fine chemical abscisic acid; or
increasing or generating the activity of an expression product of one or more nucleic acid molecule(s) comprising a polynucleotide as depicted in the respective line in column 5 or 8 of Table I, application no. 13, preferably the coding region thereof, or a homolog or a fragment thereof, whereby the respective line disclose in column 7 the fine chemical abscisic acid,
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of abscisic acid, or a composition comprising abscisic acid in said non-human organism or in the culture medium surrounding said non-human organism.

A further embodiment of the present invention relates to a process for the production of cryptoxanthin, which comprises
(a1) increasing or generating the activity of a polypeptide comprising a polypeptide as depicted in the respective line in column 5 or 8 of Table II, application no. 13, or a homolog or fragment thereof, a consensus sequence or at least one polypeptide motif as depicted in column 8 of Table IV, application no. 13, whereby the respective line disclose in column 7 the fine chemical cryptoxanthin; or
increasing or generating the activity of an expression product of one or more nucleic acid molecule(s) comprising a polynucleotide as depicted in the respective line in column 5 or 8 of Table I, application no. 13, preferably the coding region thereof, or a homolog or a fragment thereof, whereby the respective line disclose in column 7 the fine chemical cryptoxanthin;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 13, or a homolog or a fragment thereof, a consensus sequence or at least one polypeptide motif as depicted in the respective line in column 8 of Table IV, application no. 13, which is joined to a transit peptide, whereby the respective line disclose in column 7 the fine chemical cryptoxanthin; or
increasing or generating the activity of an expression product of one or more nucleic acid molecule(s) comprising a polynucleotide as depicted in the respective line in column 5 or 8 of Table I, application no. 13, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical cryptoxanthin;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 13, or a homolog or a fragment thereof, a consensus sequence or at least one polypeptide motif as depicted in the respective line in column 8 of Table IV, application no. 13, whereby the respective line disclose in column 7 the fine chemical cryptoxanthin; or
increasing or generating the activity of an expression product of one or more nucleic acid molecule(s) comprising a polynucleotide as depicted in the respective line in column 5 or 8 of Table I, application no. 13, preferably the coding region thereof, or a homolog or a fragment thereof, whereby the respective line disclose in column 7 the fine chemical cryptoxanthin;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of cryptoxanthin, or a composition comprising cryptoxanthin in said non-human organism or in the culture medium surrounding said non-human organism.

A further embodiment of the present invention relates to a process for the production of violaxanthin, which comprises
(a1) increasing or generating the activity of a polypeptide comprising a polypeptide as depicted in the respective line in column 5 or 8 of Table II, application no. 13, or a homolog or fragment thereof, a consensus sequence or at least one polypeptide motif as depicted in column 8 of Table IV, application no. 13, whereby the respective line disclose in column 7 the fine chemical violaxanthin; or
increasing or generating the activity of an expression product of one or more nucleic acid molecule(s) comprising a polynucleotide as depicted in the respective line in column 5 or 8 of Table I, application no. 13, preferably the coding region thereof, or a homolog or a fragment thereof, whereby the respective line disclose in column 7 the fine chemical violaxanthin;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 13, or a homolog or a fragment thereof, a consensus sequence or at least one polypeptide motif as depicted in the respective line in column 8 of Table IV, application no. 13, which is joined to a transit peptide, whereby the respective line disclose in column 7 the fine chemical violaxanthin; or
increasing or generating the activity of an expression product of one or more nucleic acid molecule(s) comprising a polynucleotide as depicted in the respective line in column 5 or 8 of Table I, application no. 13, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical violaxanthin;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 13, or a homolog or a fragment thereof, a consensus sequence or at least one polypeptide motif as depicted in the respective line in column 8 of Table IV, application no. 13, whereby the respective line disclose in column 7 the fine chemical violaxanthin; or
increasing or generating the activity of an expression product of one or more nucleic acid molecule(s) comprising a polynucleotide as depicted in the respective line in column 5 or 8 of Table I, application no. 13, preferably the coding region thereof, or a homolog or a fragment thereof, whereby the respective line disclose in column 7 the fine chemical violaxanthin;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of violaxanthin, or a composition comprising violaxanthin in said non-human organism or in the culture medium surrounding said non-human organism.

A further embodiment of the present invention relates to a process for the production of zeaxanthin, which comprises
(a1) increasing or generating the activity of a polypeptide comprising a polypeptide as depicted in the respective line in column 5 or 8 of Table II, application no. 13, or a homolog or fragment thereof, a consensus sequence or at least one polypeptide motif as depicted in column 8 of Table IV, application no. 13, whereby the respective line disclose in column 7 the fine chemical zeaxanthin; or
increasing or generating the activity of an expression product of one or more nucleic acid molecule(s) comprising a polynucleotide as depicted in the respective line in column 5 or 8 of Table I, application no. 13, preferably the coding region thereof, or a homolog or a fragment thereof, whereby the respective line disclose in column 7 the fine chemical zeaxanthin;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 13, or a homolog or a fragment thereof, a consensus sequence or at least one polypeptide motif as depicted in the respective line in column 8 of Table IV, application no. 13, which is joined to a transit peptide, whereby the respective line disclose in column 7 the fine chemical zeaxanthin; or
increasing or generating the activity of an expression product of one or more nucleic acid molecule(s) comprising a polynucleotide as depicted in the respective line in column 5 or 8 of Table I, application no. 13, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical zeaxanthin;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 13, or a homolog or a fragment thereof, a consensus sequence or at least one polypeptide motif as depicted in the respective line in column 8 of Table IV, application no. 13, whereby the respective line disclose in column 7 the fine chemical zeaxanthin; or
increasing or generating the activity of an expression product of one or more nucleic acid molecule(s) comprising a polynucleotide as depicted in the respective line in column 5 or 8 of Table I, application no. 13, preferably the coding region thereof, or a homolog or a fragment thereof, whereby the respective line disclose in column 7 the fine chemical zeaxanthin;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of zeaxanthin, or a composition comprising zeaxanthin in said non-human organism or in the culture medium surrounding said non-human organism.

Accordingly, the present invention relates to a process for the production of cryptoxanthin, which comprises
(a) increasing or generating the activity of a thioredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of zeaxanthin, which comprises
(a) increasing or generating the activity of a F-box protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of zeaxanthin, which comprises
(a) increasing or generating the activity of a monthiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of zeaxanthin, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of zeaxanthin, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of zeaxanthin, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of cryptoxanthin, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of zeaxanthin, which comprises
(a) increasing or generating the activity of a MADS-box transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of zeaxanthin, which comprises
(a) increasing or generating the activity of a isopropylmalate isomerase large subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of cryptoxanthin, which comprises
(a) increasing or generating the activity of a AX653549-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of zeaxanthin, which comprises
(a) increasing or generating the activity of a b1003-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of zeaxanthin, which comprises
(a) increasing or generating the activity of a lipoprotein precursor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of zeaxanthin, which comprises
(a) increasing or generating the activity of a serine/threonine-protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of zeaxanthin, which comprises
(a) increasing or generating the activity of a b2032-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of cryptoxanthin, which comprises
(a) increasing or generating the activity of a b2099-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of zeaxanthin, which comprises
(a) increasing or generating the activity of a b2121-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of zeaxanthin, which comprises
(a) increasing or generating the activity of a b2474-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of zeaxanthin, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of cryptoxanthin, which comprises
(a) increasing or generating the activity of a flavodoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of violaxanthin, which comprises
(a) increasing or generating the activity of a ribosephosphate isomerase, constitutive in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of violaxanthin or a composition comprising violaxanthin in said non-human organism or in the culture medium surrounding said non-human organism.

Accordingly, the present invention relates to a process for the production of violaxanthin, which comprises
(a) increasing or generating the activity of a acetyl CoA carboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of violaxanthin or a composition comprising violaxanthin in said non-human organism or in the culture medium surrounding said non-human organism.

Accordingly, the present invention relates to a process for the production of cryptoxanthin, which comprises
(a) increasing or generating the activity of a b3346-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of violaxanthin, which comprises
(a) increasing or generating the activity of a ketol-acid reductoisomerase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of violaxanthin or a composition comprising violaxanthin in said non-human organism or in the culture medium surrounding said non-human organism.

Accordingly, the present invention relates to a process for the production of cryptoxanthin, which comprises
(a) increasing or generating the activity of a b3817-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of violaxanthin, which comprises
(a) increasing or generating the activity of a superoxide dismutase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of violaxanthin or a composition comprising violaxanthin in said non-human organism or in the culture medium surrounding said non-human organism.

Accordingly, the present invention relates to a process for the production of violaxanthin, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of violaxanthin or a composition comprising violaxanthin in said non-human organism or in the culture medium surrounding said non-human organism.

Accordingly, the present invention relates to a process for the production of cryptoxanthin, which comprises
(a) increasing or generating the activity of a melibiose carrier protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of zeaxanthin, which comprises
(a) increasing or generating the activity of a melibiose carrier protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of violaxanthin, which comprises
(a) increasing or generating the activity of a purine-nucleoside phosphorylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of violaxanthin or a composition comprising violaxanthin in said non-human organism or in the culture medium surrounding said non-human organism.

Accordingly, the present invention relates to a process for the production of zeaxanthin, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of cryptoxanthin, which comprises
(a) increasing or generating the activity of a coproporphyrinogen oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of zeaxanthin, which comprises
(a) increasing or generating the activity of a coproporphyrinogen oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of zeaxanthin, which comprises
(a) increasing or generating the activity of a glycerol-3-phosphate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of cryptoxanthin, which comprises
(a) increasing or generating the activity of a YCL026C-A-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of cryptoxanthin, which comprises
(a) increasing or generating the activity of a F-box protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of cryptoxanthin, which comprises
(a) increasing or generating the activity of a ygl096w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of cryptoxanthin, which comprises
(a) increasing or generating the activity of a alcohol dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of cryptoxanthin, which comprises
(a) increasing or generating the activity of a halotolerance protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of zeaxanthin, which comprises
(a) increasing or generating the activity of a halotolerance protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of zeaxanthin, which comprises
(a) increasing or generating the activity of a N-acetylglucosaminylphosphatidylinositoldeacetylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of cryptoxanthin, which comprises
(a) increasing or generating the activity of a ymr324c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cryptoxanthin or a composition comprising cryptoxanthin in said non-human organism or in the culture medium surrounding said non-human organism.

Accordingly, the present invention relates to a process for the production of zeaxanthin, which comprises
(a) increasing or generating the activity of a sterol O-acyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of zeaxanthin or a composition comprising zeaxanthin in said non-human organism or in the culture medium surrounding said non-human organism.

Accordingly, the present invention relates to a process for the production of abscisic acid, which comprises
(a) increasing or generating the activity of a YER184C-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

Accordingly, the present invention relates to a process for the production of abscisic acid, which comprises
(a) increasing or generating the activity of a YOR044W-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

Accordingly, the present invention relates to a process for the production of abscisic acid, which comprises
(a) increasing or generating the activity of a gluconate transport system permease 3 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

Accordingly, the present invention relates to a process for the production of abscisic acid, which comprises
(a) increasing or generating the activity of a nitrate/nitrite transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

Accordingly, the present invention relates to a process for the production of abscisic acid, which comprises
(a) increasing or generating the activity of a acyl transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

Accordingly, the present invention relates to a process for the production of abscisic acid, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

Accordingly, the present invention relates to a process for the production of abscisic acid, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

Accordingly, the present invention relates to a process for the production of abscisic acid, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

Accordingly, the present invention relates to a process for the production of abscisic acid, which comprises
(a) increasing or generating the activity of a glycine cleavage system T aminomethyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

Accordingly, the present invention relates to a process for the production of abscisic acid, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid or a composition comprising abscisic acid in said non-human organism or in the culture medium surrounding said non-human organism.

Accordingly, the present invention relates to a process for the production of abscisic acid, which comprises
(a) increasing or generating the activity of a polypeptide comprising a polypeptide as depicted in the respective line in column 5 or 8 of Table II, application no. 13, or a homolog or a fragment thereof, a consensus sequence or at least one polypeptide motif as depicted in the respective line in column 8 of Table IV, application no. 13, whereby the respective line disclose in column 7 the fine chemical abscisic acid; or
increasing or generating the activity of an expression product of one or more nucleic acid molecule(s) comprising a polynucleotide as depicted in the respective line in column 5 or 8 of Table I, application no. 13, preferably the coding region thereof, or a homolog or a fragment thereof, whereby the respective line disclose in column 7 the fine chemical abscisic acid;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of abscisic acid, or a composition comprising abscisic acid in said non-human organism or in the culture medium surrounding said non-human organism.

Accordingly, the present invention relates to a process for the production of cryptoxanthin, which comprises
(a) increasing or generating the activity of a polypeptide comprising a polypeptide as depicted in the respective line in column 5 or 8 of Table II, application no. 13, or a homolog or a fragment thereof, a consensus sequence or at least one polypeptide motif as depicted in the respective line in column 8 of Table IV, application no. 13, whereby the respective line disclose in column 7 the fine chemical cryptoxanthin; or
increasing or generating the activity of an expression product of one or more nucleic acid molecule(s) comprising a polynucleotide as depicted in the respective line in column 5 or 8 of Table I, application no. 13, preferably the coding region thereof, or a homolog or a fragment thereof, whereby the respective line disclose in column 7 the fine chemical cryptoxanthin;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of cryptoxanthin, or a composition comprising cryptoxanthin in said non-human organism or in the culture medium surrounding said non-human organism.

Accordingly, the present invention relates to a process for the production of violaxanthin, which comprises
(a) increasing or generating the activity of a polypeptide comprising a polypeptide as depicted in the respective line in column 5 or 8 of Table II, application no. 13, or a homolog or a fragment thereof, a consensus sequence or at least one polypeptide motif as depicted in the respective line in column 8 of Table IV, application no. 13, whereby the respective line disclose in column 7 the fine chemical violaxanthin; or
increasing or generating the activity of an expression product of one or more nucleic acid molecule(s) comprising a polynucleotide as depicted in the respective line in column 5 or 8 of Table I, application no. 13, preferably the coding region thereof, or a homolog or a fragment thereof, whereby the respective line disclose in column 7 the fine chemical violaxanthin;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of violaxanthin, or a composition comprising violaxanthin in said non-human organism or in the culture medium surrounding said non-human organism.

Accordingly, the present invention relates to a process for the production of zeaxanthin, which comprises
(a) increasing or generating the activity of a polypeptide comprising a polypeptide as depicted in the respective line in column 5 or 8 of Table II, application no. 13, or a homolog or a fragment thereof, a consensus sequence or at least one polypeptide motif as depicted in the respective line in column 8 of Table IV, application no. 13, whereby the respective line disclose in column 7 the fine chemical zeaxanthin; or
increasing or generating the activity of an expression product of one or more nucleic acid molecule(s) comprising a polynucleotide as depicted in the respective line in column 5 or 8 of Table I, application no. 13, preferably the coding region thereof, or a homolog or a fragment thereof, whereby the respective line disclose in column 7 the fine chemical zeaxanthin;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of zeaxanthin, or a composition comprising zeaxanthin in said non-human organism or in the culture medium surrounding said non-human organism.

In a further embodiment the activity of the polypeptide comprising a polypeptide as depicted in the respective line in column 5 or 8 of Table II, application no. 13, or a homolog or a fragment thereof, a consensus sequence or at least one polypeptide motif as depicted in the respective line in column 8 of Table IV, application no. 13, is increased or generated non-targeted in the above-mentioned process in a microorganism or plant or a part thereof.

In a further embodiment said polypeptide has the activity of the respective polypeptide represented by a protein comprising a polypeptide as depicted in the respective line in column 5 of Table II, application no. 13.

In a further embodiment the activity of the expression product of one or more nucleic acid molecule(s) comprising a polynucleotide as depicted in the respective line in column 5 or 8 of Table I, application no. 13, preferably the coding region thereof, or a homolog or a fragment thereof, is increased or generated non-targeted in the above-mentioned process in a microorganism or plant or a part thereof.

In a further embodiment the activity of the polypeptide comprising a polypeptide as depicted in the respective line in column 5 or 8 of Table II, application no. 13, or a homolog or a fragment thereof, a consensus sequence or at least one polypeptide motif as depicted in the respective line in column 8 of Table IV, application no. 13, is increased or generated in the above-mentioned process in an organelle preferably in plastids or mitochondria, especially in plastids, of a microorganism or plant.

In a further embodiment said polypeptide has the activity of the respective polypeptide represented by a protein comprising a polypeptide as depicted in the respective line in column 5 of Table II, application no. 13.

In a further embodiment the activity of the expression product of one or more nucleic acid molecule(s) comprising a polynucleotide as depicted in the respective line in column 5 or 8 of Table I, application no. 13, preferably the coding region thereof, or a homolog or a fragment thereof, is increased or generated in the above-mentioned process in an organelle, preferably in plasids or mitochondria, especially in plastids, of a microorganism or plant.

In a further embodiment the activity of the polypeptide comprising a polypeptide as depicted in the respective line in column 5 or 8 of Table II, application no. 13, or a homolog or a fragment thereof, a consensus sequence or at least one polypeptide motif as depicted in the respective line in column 8 of Table IV, application no. 13, is increased or generated in the above-mentioned process in the cyctosol of a cell, of a microorganism or plant.

In a further embodiment said polypeptide has the activity of the respective polypeptide represented by a protein comprising a polypeptide as depicted in the respective line in column 5 of Table II, application no. 13.

In a further embodiment the activity of the expression product of one or more nucleic acid molecule(s) comprising a polynucleotide as depicted in the respective line in column 5 or 8 of Table I, application no. 13, preferably the coding region thereof, or a homolog or a fragment thereof, is increased or generated in the above-mentioned process in the cytosol of a cell, of a microorganism or plant.

[0039.1.7.13] to [0066.1.7.13] for the disclosure of these paragraphs see [0039.1.7.7] [0066.1.7.7] above

As mentioned above the nucleic acid sequences coding for the proteins as shown in Table II, application no. 13, column 5 or 8, or homologs or fragments thereof, are joined to a nucleic acid sequence encoding a plastidic transit peptide, e.g, if for the nucleic acid molecule in Table I, column 6 the term “plastidic” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide coding sequence is fused in frame to the nucleic acid sequence coding for proteins as shown in Table II, application no. 13, column 5 or 8, or homologs or fragments thereof, e.g., if for the nucleic acid molecule in Table I, column 6 the term “plastidic” is indicated.

[0068.1.7.13] to [0072.1.7.13] for the disclosure of these paragraphs see [0068.1.7.7] to [0072.1.7.7] above

As mentioned above the nucleic acid sequences coding for the proteins as shown in the respective line in Table II, application no. 13, column 5 or 8, or homologs or fragments thereof, are joined to a nucleic acid sequence encoding a mitochondric transit peptide, e.g., if for the respective nucleic acid molecule in Table I, column 6 the term “mitochondric” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in the respective line in Table II, application no. 13, column 5 or 8, or homologs or fragments thereof, e.g., if for the nucleic acid molecule in the respective line in Table I, column 6 the term “mitochondric” is indicated.

for the disclosure of this paragraph see [0074.1.7.7] above

for the disclosure of this paragraph see [0075.1.7.7] above

Nucleic acid sequences coding for the transit peptides may be chemically synthesized either in part or wholly according to structure of transit peptide sequences disclosed in the prior art. Said natural or chemically synthesized sequences can be directly linked to the sequences encoding the mature protein or via a linker nucleic acid sequence, which may be typically less than 500 base pairs, preferably less than 450, 400, 350, 300, 250 or 200 base pairs, more preferably less than 150, 100, 90, 80, 70, 60, 50, 40 or 30 base pairs and most preferably less than 25, 20, 15, 12, 9, 6 or 3 base pairs in length and are in frame to the coding sequence. Furthermore favorable nucleic acid sequences encoding transit peptides may comprise sequences derived from more than one biological and/or chemical source and may include a nucleic acid sequence derived from the amino-terminal region of the mature protein, which in its native state is linked to the transit peptide. In a preferred embodiment of the invention said amino-terminal region of the mature protein is typically less than 150 amino acids, preferably less than 140, 130, 120, 110, 100 or 90 amino acids, more preferably less than 80, 70, 60, 50, 40, 35, 30, 25 or 20 amino acids and most preferably less than 19, 18, 17, 16, 15, 14, 13, 12, 11 or 10 amino acids in length. But even shorter or longer stretches are also possible. In addition target sequences, which facilitate the transport of proteins to other cell compartments such as the vacuole, endoplasmic reticulum, golgi complex, glyoxysomes, peroxisomes, mitochondria (in case of a plastidal transit peptide) or plastids, especially chloroplasts (in case of a mitochondric transit peptide) may be also part of the inventive nucleic acid sequence. The proteins translated from said inventive nucleic acid sequences are a kind of fusion proteins that means the nucleic acid sequences encoding the plastidal transit peptide for example the ones shown in Table a, preferably the last one of the Table a are joint to the respective nucleic acid sequences shown in Table I, application no. 13, columns 5 or 8, especially the coding region thereof, or homologs or fragments thereof, in case of nucleic acid sequences being expressed in plastids, and that means the nucleic acid sequences encoding the mitochondric transit peptide for example the ones shown in Table b, preferably the last one of the Table b are joint to the respective nucleic acid sequences shown in Table I, application no. 13, columns 5 or 8, especially the coding region thereof, or homologs or fragments thereof, in case of nucleic acid sequences being expressed in mitochondria. The person skilled in the art is able to join said sequences in a functional manner. Advantageously the transit peptide part is cleaved off from the protein part shown in Table II, application no. 13, columns 5 or 8, or a homolog or fragment thereof, during or after the transport into the organelles, preferably into the plastids or mitochondria, respectively.

All products of the cleavage of the preferred transit peptide shown in the last line of Table a have preferably the N-terminal amino acid sequences QIA CSS or QIA EFQLTT in front of the start methionine of the respective protein mentioned in Table II, application no. 13, column 5 or 8. or homologs or fragments thereof. Other short amino acid sequences of an range of 1 to 20 amino acids preferable 2 to 15 amino acids, more preferable 3 to 10 amino acids most preferably 4 to 8 amino acids are also possible in front of the start methionine of the protein mentioned in Table II, application no. 13, columns 5 or 8, or homologs or fragments thereof. In case of the amino acid sequence QIA CSS the three amino acids in front of the start methionine are stemming from the LIC (=ligatation independent cloning) cassette. Said short amino acid sequence is preferred in the case of the expression of E. coli genes. In case of the amino acid sequence QIA EFQLTT the six amino acids in front of the start methionine are stemming from the LIC cassette. Said short amino acid sequence is preferred in the case of the expression of S. cerevisiae genes. The skilled worker knowns that other short sequences are also useful in the expression of the respective nucleic acid molecule comprising a polynucleotide as depicted in Table I, application no. 13, column 5 or 8, preferably the coding region thereof, or a homolog or a fragment thereof. Furthermore the skilled worker is aware of the fact that there is not a need for such short sequences in the expression of the genes.

for the disclosure of this paragraph see [0077.1.7.7] above

for the disclosure of this paragraph see [0078.1.7.7] above

Alternatively to the targeting of the respective sequences shown in Table II, application no. 13, columns 5 or 8, or homologs or fragments thereof, preferably of sequences in general encoded in the nucleus with the aid of the targeting sequences mentioned for example in Table a alone, Table b alone, or in combination with other targeting sequences preferably into plastids or mitochondria, respectively, the nucleic acids of the invention can directly be introduced into the plastidal or mitochondrial genome. Therefore in a preferred embodiment the respective nucleic acid sequences shown in the respective line in Table I, application no. 13, columns 5 or 8, preferably the coding region thereof, or homologs or fragments thereof, are directly introduced and expressed in plastids e.g., if for the nucleic acid molecule in the respective line in Table I, column 6, the term “plastidic” is indicated, or are directly introduced and expressed in mitochondria e.g., if for the nucleic acid molecule in the respective line in Table I, column 6 the term “mitochondric” is indicated.

The term “introduced” in the context of this specification shall mean the insertion of a nucleic acid sequence into the organism by means of a “transfection”, “transduction” or preferably by “transformation”.

A plastid, such as a chloroplast, or a mitochondrion, has been “transformed” by an exogenous (preferably foreign) nucleic acid sequence if a nucleic acid sequence has been introduced into the plastid or mitochondrion that means that this sequence has crossed the membrane or the membranes of the plastid or of the mitochondrion. The exogenous, preferably foreign, DNA may be integrated (covalently linked) into plastid or mitochondrial DNA making up the genome of the plastid or mitochondrion, or it may remain unintegrated (e.g., by including a chloroplast origin of replication). “Stably” integrated DNA sequences are those, which are inherited through plastid or mitochondrion replication, thereby transferring new plastids or mitochondria, with the features of the integrated DNA sequence to the progeny.

[0080.1.7.13] to [0083.1.7.13] for the disclosure of these paragraphs see [0080.1.7.7] to [0083.1.7.7] above

For the process of the present invention it may be of great advantage that by transforming the plastids the intraspecies specific transgene flow is blocked, because a lot of species such as corn, cotton and rice have a strict maternal inheritance of plastids. By placing the respective genes specified in the respective line in Table I, application no. 13, columns 5 or 8, preferably the coding region, or homologs or fragments thereof, e.g. if for the nucleic acid molecule in the respective line in column 6 of Table I the term “plastidic” is indicated, in the plastids of plants, these genes will not be present in the pollen of said plants.

A further preferred embodiment of the invention relates to the use of so called “chloroplast localization sequences”, in which a first RNA sequence or molecule is capable of transporting or “chaperoning” a second RNA sequence, such as a RNA sequence transcribed from the sequences depicted in the respective line in Table I, application no. 13, columns 5 or 8, preferably the coding region thereof, or a homolog or a fragment thereof, or a sequence encoding a protein, as depicted in the respective line in Table II, application no. 13, columns 5 and 8, or a homolog or a fragment thereof, from an external environment inside a cell or outside a plastid into a chloroplast. In one embodiment the chloroplast localization signal is substantially similar or complementary to a complete or intact viroid sequence. The chloroplast localization signal may be encoded by a DNA sequence, which is transcribed into the chloroplast localization RNA. The term “viroid” refers to a naturally occurring single stranded RNA molecule (Flores, C. R. Acad. Sci. III. 324 (10), 943 (2001)). Viroids usually contain about 200-500 nucleotides and generally exist as circular molecules. Examples of viroids that contain chloroplast localization signals include but are not limited to ASBVd, PLMVd, CChMVd and ELVd. The viroid sequence or a functional part of it can be fused to the sequences depicted in the respective line in Table I, application no. 13, columns 5 or 8, preferably the coding region thereof, or a homolog or a fragment thereof, or a sequence encoding a protein, as depicted in the respective line in Table II, application no. 13, columns 5 or 8, or a homolog or a fragment thereof, in such a manner that the viroid sequence transports a sequence transcribed from a sequence as depicted in the respective line in Table I, application no. 13 columns 5 or 8, preferably the coding region thereof, or a homolog or a fragment thereof, or a sequence encoding a protein as depicted in the respective line in Table II, application no. 13 columns 5 or 8, or a homolog or a fragment thereof, into the chloroplasts, e.g. if for the nucleic acid molecule in column 6 of Table I the term “plastidic” is indicated. A preferred embodiment uses a modified ASBVd (Navarro et al., Virology. 268 (1), 218 (2000)).

In a further specific embodiment the protein to be expressed in the plastids such as the proteins depicted in the respective line in Table II, application no. 13, columns 5 or 8, or a homolog or a fragment thereof, e.g. if for the nucleic acid molecule in the respective line in column 6 of Table I the term “plastidic” is indicated, are encoded by different nucleic acids. Such a method is disclosed in WO 2004/040973, which shall be incorporated by reference. WO 2004/040973 teaches a method, which relates to the translocation of an RNA corresponding to a gene or gene fragment into the chloroplast by means of a chloroplast localization sequence. The genes, which should be expressed in the plant or plant cells, are split into nucleic acid fragments, which are introduced into different compartments in the plant e.g. the nucleus, the plastids and/or mitochondria. Additionally plant cells are described in which the chloroplast contains a ribozyme fused at one end to an RNA encoding a fragment of a protein used in the inventive process such that the ribozyme can trans-splice the translocated fusion RNA to the RNA encoding the gene fragment to form and as the case may be reunite the nucleic acid fragments to an intact mRNA encoding a functional protein for example as disclosed in the respective line in Table II, application no. 13, columns 5 or 8, or a homolog or a fragment thereof.

In a preferred embodiment of the invention the nucleic acid sequences as shown in the respective line in Table I, application no. 13, columns 5 or 8, preferably the coding region thereof, or homologs or fragments thereof, e.g. if for the nucleic acid molecule in the respective line in column 6 of Table I the term “plastidic” is indicated, used in the inventive process are transformed into plastids, which are metabolically active. Those plastids should preferably maintain at a high copy number in the plant or plant tissue of interest, most preferably the chloroplasts found in green plant tissues, such as leaves or cotyledons or in seeds.

For a good expression in the plastids the nucleic acid sequences as shown in the respective line in Table I, application no. 13, columns 5 or 8, preferably the coding region thereof, or homologs or fragments thereof, e.g. if for the nucleic acid molecule in the respective line in column 6 of Table I the term “plastidic” is indicated, are introduced into an expression cassette using preferably a promoter and terminater, which are active in plastids preferably a chloroplast promoter. Examples of such promoters include the psbA promoter from the gene from spinach or pea, the rbcL promoter, and the atpB promoter from corn.

[0089.1.7.13] to [0092.1.7.13] for the disclosure of these paragraphs see [0089.1.7.7] to [0092.1.7.7] above

Advantageously the process for the production of the fine chemical abscisic acid, cryptoxanthin, violaxanthin, and/or zeaxanthin leads to an enhanced production of the respective fine chemical. The terms “enhanced” or “increase” mean at least a 10%, 20%, 30%, 40% or 50%, preferably at least 60%, 70%, 80%, 90% or 100%, more preferably 150%, 200%, 300%, 400% or 500% higher production of the respective fine chemical abscisic acid, cryptoxanthin, violaxanthin, and/or zeaxanthin in comparison to the wild-type as defined above, e.g. that means in comparison to a non-human organism without the aforementioned modification of the activity of a protein as shown in the respective line in Table II, application no. 13, column 5 or 8, or a fragment or a homolog thereof. The modification of the activity of a protein as shown in the respective line in Table II, application no. 13, column 5 or 8, or a homolog or a fragment thereof, or their combination can be achieved by joining the protein to a respective transit peptide, e.g. if for the respective encoding nucleic acid molecule in column 6 of Table I the term “plastidic” or “mitochondric” is indicated.

Surprisingly it was found, that the transgenic expression of a protein as shown in the respective line in Table II, application no. 13, column 5, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, conferred a production of or an increase in abscisic acid, cryptoxan thin, violaxanthin, and/or zeaxanthin,respectively, to the transgenic non-human organism as compared to a corresponding non-transformed wild type.

In one embodiment the transgenic expression of a protein as shown in the respective line in Table II, application no. 13, column 5 or 8, or a homolog or a fragment thereof, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, confers a production of or an increase in abscisic acid, cryptoxanthin, violaxanthin, and/or zeaxanthin,respectively, to the transgenic non-human organism as compared to a corresponding non-transformed wild type.

Accordingly, in one embodiment, an increase of cryptoxanthin in a non-human organism, as compared to a corresponding non-transformed wild type non-human organism, is conferred in the process of the invention, if the activity of a polypeptide showing the activity of a thioredoxin, or if the activity of the polypeptide At3g15360, preferably represented by SEQ ID NO. 130492, or a homolog or fragment thereof, or if the activity of a polypeptide encoded by a nucleic acid molecule comprising the nucleic acid SEQ ID NO. 130491, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 130491 or polypeptide SEQ ID NO. 130492, respectively, or a homolog or a fragment thereof, is increased or generated, or if the activity thioredoxin is increased or generated in a non-human organism, like a microorganism or a plant cell, plant or part thereof, especially with non-targeted localization, whereby the respective line disclose in column 7 the fine chemical cryptoxanthin. For example, an increase of the cryptoxanthin of at least 1 percent, particularly in a range of 36 to 59-percent is conferred as compared to a corresponding non-transformed wild type non-human organism.

Accordingly, in one embodiment, an increase of zeaxanthin in a non-human organism, as compared to a corresponding non-transformed wild type non-human organism, is conferred in the process of the invention, if the activity of a polypeptide showing the activity of a F-box protein, or if the activity of the polypeptide At3g16820, preferably represented by SEQ ID NO. 120953, or a homolog or fragment thereof, or if the activity of a polypeptide encoded by a nucleic acid molecule comprising the nucleic acid SEQ ID NO. 120952, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 120952 or polypeptide SEQ ID NO. 120953, respectively, or a homolog or a fragment thereof, is increased or generated, or if the activity F-box protein is increased or generated in a non-human organism, like a microorganism or a plant cell, plant or part thereof, especially with non-targeted localization, whereby the respective line disclose in column 7 the fine chemical zeaxanthin. For example, an increase of the zeaxanthin of at least 1 percent, particularly in a range of 24 to 36-percent is conferred as compared to a corresponding non-transformed wild type non-human organism.

Accordingly, in one embodiment, an increase of zeaxanthin in a non-human organism, as compared to a corresponding non-transformed wild type non-human organism, is conferred in the process of the invention, if the activity of a polypeptide showing the activity of a monthiol glutaredoxin, or if the activity of the polypeptide At3g62930, preferably represented by SEQ ID NO. 2574, or a homolog or fragment thereof, or if the activity of a polypeptide encoded by a nucleic acid molecule comprising the nucleic acid SEQ ID NO. 2573, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 2573 or polypeptide SEQ ID NO. 2574, respectively, or a homolog or a fragment thereof, is increased or generated, or if the activity monthiol glutaredoxin is increased or generated in a non-human organism, like a microorganism or a plant cell, plant or part thereof, especially with non-targeted localization, whereby the respective line disclose in column 7 the fine chemical zeaxanthin. For example, an increase of the zeaxanthin of at least 1 percent, particularly in a range of 23 to 57-percent is conferred as compared to a corresponding non-transformed wild type non-human organism.

Accordingly, in one embodiment, an increase of zeaxanthin in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical zeaxanthin. For example, an increase of the zeaxanthin of at least 1 percent, particularly in a range of 23 to 53-percent is conferred as compared to a corresponding non-transformed wild type non-human organism. Accordingly, in one embodiment, an increase of zeaxanthin in a non-human organism, as compared to a corresponding non-transformed wild type non-human organism, is conferred in the process of the invention, if the activity of a polypeptide showing the activity of a monothiol glutaredoxin, or if the activity of the polypeptide At4g15670, preferably represented by SEQ ID NO. 3280, or a homolog or fragment thereof, or if the activity of a polypeptide encoded by a nucleic acid molecule comprising the nucleic acid SEQ ID NO. 3279, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 3279 or polypeptide SEQ ID NO. 3280, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical zeaxanthin. For example, an increase of the zeaxanthin of at least 1 percent, particularly in a range of 26 to 60-percent is conferred as compared to a corresponding non-transformed wild type non-human organism.

Accordingly, in one embodiment, an increase of zeaxanthin in a non-human organism, as compared to a corresponding non-transformed wild type non-human organism, is conferred in the process of the invention, if the activity of a polypeptide showing the activity of a protein phosphatase, or if the activity of the polypeptide At4g26080, preferably represented by SEQ ID NO. 68850, or a homolog or fragment thereof, or if the activity of a polypeptide encoded by a nucleic acid molecule comprising the nucleic acid SEQ ID NO. 68849, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 68849 or polypeptide SEQ ID NO. 68850, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical zeaxanthin. For example, an increase of the zeaxanthin of at least 1 percent, particularly in a range of 23 to 41-percent is conferred as compared to a corresponding non-transformed wild type non-human organism. Accordingly, in one embodiment, an increase of cryptoxanthin in a non-human organism, as compared to a corresponding non-transformed wild type non-human organism, is conferred in the process of the invention, if the activity of a polypeptide showing the activity of a glutaredoxin, or if the activity of the polypeptide At4g33040, preferably represented by SEQ ID NO. 4103, or a homolog or fragment thereof, or if the activity of a polypeptide encoded by a nucleic acid molecule comprising the nucleic acid SEQ ID NO. 4102, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 4102 or polypeptide SEQ ID NO. 4103, respectively, or a homolog or a fragment thereof, is increased or generated, or if the activity glutaredoxin is increased or generated in a non-human organism, like a microorganism organism or a plant cell, plant or part thereof, especially with non-targeted localization, whereby the respective line disclose in column 7 the fine chemical cryptoxanthin. For example, an increase of the cryptoxanthin of at least 1 percent, particularly in a range of 32 to 64-percent is conferred as compared to a corresponding non-transformed wild type non-human organism.

Accordingly, in one embodiment, an increase of zeaxanthin in a non-human organism, as compared to a corresponding non-transformed wild type non-human organism, is conferred in the process of the invention, if the activity of a polypeptide showing the activity of a MADS-box transcription factor, or if the activity of the polypeptide At5g60440, preferably represented by SEQ ID NO. 102900, or a homolog or fragment thereof, or if the activity of a polypeptide encoded by a nucleic acid molecule comprising the nucleic acid SEQ ID NO. 102899, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 102899 or polypeptide SEQ ID NO. 102900, respectively, or a homolog or a fragment thereof, is increased or generated, or if the activity MADS-box transcription factor is increased or generated in a non-human organism, like a microorganism or a plant cell, plant or part thereof, especially with non-targeted localization, whereby the respective line disclose in column 7 the fine chemical zeaxanthin. For example, an increase of the zeaxanthin of at least 1 percent, particularly in a range of 25 to 46-percent is conferred as compared to a corresponding non-transformed wild type non-human organism.

Accordingly, in one embodiment, an increase of zeaxanthin in a non-human organism, as compared to a corresponding non-transformed wild type non-human organism, is conferred in the process of the invention, if the activity of a polypeptide showing the activity of a isopropylmalate isomerase large subunit, or if the activity of the polypeptide AvinDRAFT_{—}4847, preferably represented by SEQ ID NO. 102942, or a homolog or fragment thereof, or if the activity of a polypeptide encoded by a nucleic acid molecule comprising the nucleic acid SEQ ID NO. 102941, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 102941 or polypeptide SEQ ID NO. 102942, respectively, or a homolog or a fragment thereof, is increased or generated, or if the activity isopropylmalate isomerase large subunit is increased or generated in a non-human organism, like a microorganism or a plant cell, plant or part thereof, especially with non-targeted localization, whereby the respective line disclose in column 7 the fine chemical zeaxanthin. For example, an increase of the zeaxanthin of at least 1 percent, particularly in a range of 36 to 46-percent is conferred as compared to a corresponding non-transformed wild type non-human organism.

Accordingly, in one embodiment, an increase of cryptoxanthin in a non-human organism, as compared to a corresponding non-transformed wild type non-human organism, is conferred in the process of the invention, if the activity of a polypeptide showing the activity of a AX653549-protein, or if the activity of the polypeptide AX653549, preferably represented by SEQ ID NO. 34302, or a homolog or fragment thereof, or if the activity of a polypeptide encoded by a nucleic acid molecule comprising the nucleic acid SEQ ID NO. 34301, preferably the coding region thereof, or a homolog or fragment thereof, e.g. derived from Oryza sativa, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 34301 or polypeptide SEQ ID NO. 34302, respectively, or a homolog or a fragment thereof, is increased or generated, or if the activity AX653549-protein is increased or generated in a non-human organism, like a microorganism or a plant cell, plant or part thereof, especially with non-targeted localization, whereby the respective line disclose in column 7 the fine chemical cryptoxanthin. For example, an increase of the cryptoxanthin of at least 1 percent, particularly in a range of 34 to 52-percent is conferred as compared to a corresponding non-transformed wild type non-human organism.

Accordingly, in one embodiment, an increase of zeaxanthin in a non-human organism, as compared to a corresponding non-transformed wild type non-human organism, is conferred in the process of the invention, if the activity of a polypeptide showing the activity of a b1003-protein, or if the activity of the polypeptide B1003, preferably represented by SEQ ID NO. 7942, or a homolog or fragment thereof, or if the activity of a polypeptide encoded by a nucleic acid molecule comprising the nucleic acid SEQ ID NO. 7941, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 7941 or polypeptide SEQ ID NO. 7942, respectively, or a homolog or a fragment thereof, is increased or generated, or if the activity b1003-protein is increased or generated in a non-human organism, like a microorganism or a plant cell, plant or part thereof, especially with non-targeted localization, whereby the respective line disclose in column 7 the fine chemical zeaxanthin. For example, an increase of the zeaxanthin of at least 1 percent, particularly in a range of 26 to 51-percent is conferred as compared to a corresponding non-transformed wild type non-human organism.

Accordingly, in one embodiment, an increase of zeaxanthin in a non-human organism, as compared to a corresponding non-transformed wild type non-human organism, is conferred in the process of the invention, if the activity of a polypeptide showing the activity of a lipoprotein precursor, or if the activity of the polypeptide B1023, preferably represented by SEQ ID NO. 36881, or a homolog or fragment thereof, or if the activity of a polypeptide encoded by a nucleic acid molecule comprising the nucleic acid SEQ ID NO. 36880, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 36880 or polypeptide SEQ ID NO. 36881, respectively, or a homolog or a fragment thereof, is increased or generated, or if the activity lipoprotein precursor is increased or generated in a non-human organism, like a microorganism or a plant cell, plant or part thereof, especially with non-targeted localization, whereby the respective line disclose in column 7 the fine chemical zeaxanthin. For example, an increase of the zeaxanthin of at least 1 percent, particularly in a range of 27 to 46-percent is conferred as compared to a corresponding non-transformed wild type non-human organism.

Accordingly, in one embodiment, an increase of zeaxanthin in a non-human organism, as compared to a corresponding non-transformed wild type non-human organism, is conferred in the process of the invention, if the activity of a polypeptide showing the activity of a serine/threonine-protein phosphatase, or if the activity of the polypeptide B1838, preferably represented by SEQ ID NO. 8317, or a homolog or fragment thereof, or if the activity of a polypeptide encoded by a nucleic acid molecule comprising the nucleic acid SEQ ID NO. 8316, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 8316 or polypeptide SEQ ID NO. 8317, respectively, or a homolog or a fragment thereof, is increased or generated, or if the activity serine/threonine-protein phosphatase is increased or generated in a non-human organism, like a microorganism or a plant cell, plant or part thereof, especially with non-targeted localization, whereby the respective line disclose in column 7 the fine chemical zeaxanthin. For example, an increase of the zeaxanthin of at least 1 percent, particularly in a range of 23 to 47-percent is conferred as compared to a corresponding non-transformed wild type non-human organism.

Accordingly, in one embodiment, an increase of zeaxanthin in a non-human organism, as compared to a corresponding non-transformed wild type non-human organism, is conferred in the process of the invention, if the activity of a polypeptide showing the activity of a b2032-protein, or if the activity of the polypeptide B2032, preferably represented by SEQ ID NO. 8921, or a homolog or fragment thereof, or if the activity of a polypeptide encoded by a nucleic acid molecule comprising the nucleic acid SEQ ID NO. 8920, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 8920 or polypeptide SEQ ID NO. 8921, respectively, or a homolog or a fragment thereof, is increased or generated, or if the activity b2032-protein is increased or generated in a non-human organism, like a microorganism or a plant cell, plant or part thereof, especially with non-targeted localization, whereby the respective line disclose in column 7 the fine chemical zeaxanthin. For example, an increase of the zeaxanthin of at least 1 percent, particularly in a range of 27 to 59-percent is conferred as compared to a corresponding non-transformed wild type non-human organism.

Accordingly, in one embodiment, an increase of cryptoxanthin in a non-human organism, as compared to a corresponding non-transformed wild type non-human organism, is conferred in the process of the invention, if the activity of a polypeptide showing the activity of a b2099-protein, or if the activity of the polypeptide B2099, preferably represented by SEQ ID NO. 115596, or a homolog or fragment thereof, or if the activity of a polypeptide encoded by a nucleic acid molecule comprising the nucleic acid SEQ ID NO. 115595, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 115595 or polypeptide SEQ ID NO. 115596, respectively, or a homolog or a fragment thereof, is increased or generated, or if the activity b2099-protein is increased or generated in a non-human organism, like a microorganism or a plant cell, plant or part thereof, especially with non-targeted localization, whereby the respective line disclose in column 7 the fine chemical cryptoxanthin. For example, an increase of the cryptoxanthin of at least 1 percent, particularly in a range of 32 to 53-percent is conferred as compared to a corresponding non-transformed wild type non-human organism.

Accordingly, in one embodiment, an increase of zeaxanthin in a non-human organism, as compared to a corresponding non-transformed wild type non-human organism, is conferred in the process of the invention, if the activity of a polypeptide showing the activity of a b2121-protein, or if the activity of the polypeptide B2121, preferably represented by SEQ ID NO. 39014, or a homolog or fragment thereof, or if the activity of a polypeptide encoded by a nucleic acid molecule comprising the nucleic acid SEQ ID NO. 39013, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 39013 or polypeptide SEQ ID NO. 39014, respectively, or a homolog or a fragment thereof, is increased or generated, or if the activity b2121-protein is increased or generated in a non-human organism, like a microorganism or a plant cell, plant or part thereof, especially with non-targeted localization, whereby the respective line disclose in column 7 the fine chemical zeaxanthin. For example, an increase of the zeaxanthin of at least 1 percent, particularly in a range of 37 to 40-percent is conferred as compared to a corresponding non-transformed wild type non-human organism.

Accordingly, in one embodiment, an increase of zeaxanthin in a non-human organism, as compared to a corresponding non-transformed wild type non-human organism, is conferred in the process of the invention, if the activity of a polypeptide showing the activity of a b2474-protein, or if the activity of the polypeptide B2474, preferably represented by SEQ ID NO. 40330, or a homolog or fragment thereof, or if the activity of a polypeptide encoded by a nucleic acid molecule comprising the nucleic acid SEQ ID NO. 40329, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 40329 or polypeptide SEQ ID NO. 40330, respectively, or a homolog or a fragment thereof, is increased or generated, or if the activity b2474-protein is increased or generated in a non-human organism, like a microorganism or a plant cell, plant or part thereof, especially with non-targeted localization, whereby the respective line disclose in column 7 the fine chemical zeaxanthin. For example, an increase of the zeaxanthin of at least 1 percent, particularly in a range of 22 to 42-percent is conferred as compared to a corresponding non-transformed wild type non-human organism.

Accordingly, in one embodiment, an increase of zeaxanthin in a non-human organism, as compared to a corresponding non-transformed wild type non-human organism, is conferred in the process of the invention, if the activity of a polypeptide showing the activity of a transcriptional regulator, or if the activity of the polypeptide B2714, preferably represented by SEQ ID NO. 40742, or a homolog or fragment thereof, or if the activity of a polypeptide encoded by a nucleic acid molecule comprising the nucleic acid SEQ ID NO. 40741, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 40741 or polypeptide SEQ ID NO. 40742, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line disclose in column 7 the fine chemical zeaxanthin. For example, an increase of the zeaxanthin of at least 1 percent, particularly in a range of 23 to 38-percent is conferred as compared to a corresponding non-transformed wild type non-human organism.

Accordingly, in one embodiment, an increase of cryptoxanthin in a non-human organism, as compared to a corresponding non-transformed wild type non-human organism, is conferred in the process of the invention, if the activity of a polypeptide showing the activity of a flavodoxin, or if the activity of the polypeptide B2790, preferably represented by SEQ ID NO. 115720, or a homolog or fragment thereof, or if the activity of a polypeptide encoded by a nucleic acid molecule comprising the nucleic acid SEQ ID NO. 115719, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 115719 or polypeptide SEQ ID NO. 115720, respectively, or a homolog or a fragment thereof, is increased or generated, or if the activity flavodoxin is increased or generated in a non-human organism, like a microorganism or a plant cell, plant or part thereof, especially with non-targeted localization, whereby the respective line disclose in column 7 the fine chemical cryptoxanthin. For example, an increase of the cryptoxanthin of at least 1 percent, particularly in a range of 29 to 43-percent is conferred as compared to a corresponding non-transformed wild type non-human organism.

Accordingly, in one embodiment, an increase of violaxanthin in a non-human organism, as compared to a corresponding non-transformed wild type non-human organism, is conferred in the process of the invention, if the activity of a polypeptide showing the activity of a ribosephosphate isomerase, constitutive, or if the activity of the polypeptide B2914, preferably represented by SEQ ID NO. 41074, or a homolog or fragment thereof, or if the activity of a polypeptide encoded by a nucleic acid molecule comprising the nucleic acid SEQ ID NO. 41073, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 41073 or polypeptide SEQ ID NO. 41074, respectively, or a homolog or a fragment thereof, is increased or generated, or if the activity ribosephosphate isomerase, constitutive is increased or generated in a non-human organism, like a microorganism or a plant cell, plant or part thereof, especially with non-targeted localization, whereby the respective line disclose in column 7 the fine chemical violaxanthin. For example, an increase of the violaxanthin of at least 1 percent, particularly in a range of 50 to 220-percent is conferred as compared to a corresponding non-transformed wild type non-human organism.

Accordingly, in one embodiment, an increase of violaxanthin in a non-human organism, as compared to a corresponding non-transformed wild type non-human organism, is conferred in the process of the invention, if the activity of a polypeptide showing the activity of a acetyl CoA carboxylase, or if the activity of the polypeptide B3256, preferably represented by SEQ ID NO. 9493, or a homolog or fragment thereof, or if the activity of a polypeptide encoded by a nucleic acid molecule comprising the nucleic acid SEQ ID NO. 9492, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 9492 or polypeptide SEQ ID NO. 9493, respectively, or a homolog or a fragment thereof, is increased or generated, or if the activity acetyl CoA carboxylase is increased or generated in a non-human organism, like a microorganism or a plant cell, plant or part thereof, especially with non-targeted localization, whereby the respective line disclose in column 7 the fine chemical violaxanthin. For example, an increase of the violaxanthin of at least 1 percent, particularly in a range of 36 to 220-percent is conferred as compared to a corresponding non-transformed wild type non-human organism.

Accordingly, in one embodiment, an increase of cryptoxanthin in a non-human organism, as compared to a corresponding non-transformed wild type non-human organism, is conferred in the process of the invention, if the activity of a polypeptide showing the activity of a b3346-protein, or if the activity of the polypeptide B3346, preferably represented by SEQ ID NO. 10105, or a homolog or fragment thereof, or if the activity of a polypeptide encoded by a nucleic acid molecule comprising the nucleic acid SEQ ID NO. 10104, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 10104 or polypeptide SEQ ID NO. 10105, respectively, or a homolog or a fragment thereof, is increased or generated, or if the activity b3346-protein is increased or generated in a non-human organism, like a microorganism or a plant cell, plant or part thereof, especially with non-targeted localization, whereby the respective line disclose in column 7 the fine chemical cryptoxanthin. For example, an increase of the cryptoxanthin of at least 1 percent, particularly in a range of 47 to 60-percent is conferred as compared to a corresponding non-transformed wild type non-human organism.

Accordingly, in one embodiment, an increase of violaxanthin in a non-human organism, as compared to a corresponding non-transformed wild type non-human organism, is conferred in the process of the invention, if the activity of a polypeptide showing the activity of a ketol-acid reductoisomerase, or if the activity of the polypeptide B3774, preferably represented by SEQ ID NO. 94387, or a homolog or fragment thereof, or if the activity of a polypeptide encoded by a nucleic acid molecule comprising the nucleic acid SEQ ID NO. 94386, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 94386 or polypeptide SEQ ID NO. 94387, respectively, or a homolog or a fragment thereof, is increased or generated, or if the activity ketol-acid reductoisomerase is increased or generated in a non-human organism , like a microorganism or a plant cell, plant or part thereof, especially with non-targeted localization, whereby the respective line disclose in column 7 the fine chemical violaxanthin. For example, an increase of the violaxanthin of at least 1 percent, particularly in a range of 35 to 232-percent is conferred as compared to a corresponding non-transformed wild type non-human organism.

Accordingly, in one embodiment, an increase of cryptoxanthin in a non-human organism, as compared to a corresponding non-transformed wild type non-human organism, is conferred in the process of the invention, if the activity of a polypeptide showing the activity of a b3817-protein, or if the activity of the polypeptide B3817, preferably represented by SEQ ID NO. 10709, or a homolog or fragment thereof, or if the activity of a polypeptide encoded by a nucleic acid molecule comprising the nucleic acid SEQ ID NO. 10708, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 10708 or polypeptide SEQ ID NO. 10709, respectively, or a homolog or a fragment thereof, is increased or generated, or if the activity b3817-protein is increased or generated in a non-human organism, like a microorganism or a plant cell, plant or part thereof, especially with non-targeted localization, whereby the respective line disclose in column 7 the fine chemical cryptoxanthin. For example, an increase of the cryptoxanthin of at least 1 percent, particularly in a range of 34 to 66-percent is conferred as compared to a corresponding non-transformed wild type non-human organism.

Accordingly, in one embodiment, an increase of violaxanthin in a non-human organism, as compared to a corresponding non-transformed wild type non-human organism, is conferred in the process of the invention, if the activity of a polypeptide showing the activity of a superoxide dismutase, or if the activity of the polypeptide B3908, preferably represented by SEQ ID NO. 105660, or a homolog or fragment thereof, or if the activity of a polypeptide encoded by a nucleic acid molecule comprising the nucleic acid SEQ ID NO. 105659, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 105659 or polypeptide SEQ ID NO. 105660, respectively, or a homolog or a fragment thereof, is increased or generated, or if the activity superoxide dismutase is increased or generated in a non-human organism, like a microorganism or a plant cell, plant or part thereof, especially with plastidic localization, whereby the respective line disclose in column 7 the fine chemical violaxanthin. For example, an increase of the violaxanthin of at least 1 percent, particularly in a range of 47 to 66-percent is conferred as compared to a corresponding non-transformed wild type non-human organism.

Accordingly, in one embodiment, an increase of violaxanthin in a non-human organism, as compared to a corresponding non-transformed wild type non-human organism, is conferred in the process of the invention, if the activity of a polypeptide showing the activity of a acetyltransferase, or if the activity of the polypeptide B4012, preferably represented by SEQ ID NO. 44379, or a homolog or fragment thereof, or if the activity of a polypeptide encoded by a nucleic acid molecule comprising the nucleic acid SEQ ID NO. 44378, preferably the coding region thereof, or a homolog or fragment thereof, e.g. derived from Escherichia coli, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 44378 or polypeptide SEQ ID NO. 44379, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line disclose in column 7 the fine chemical violaxanthin. For example, an increase of the violaxanthin of at least 1 percent, particularly in a range of 39 to 118-percent is conferred as compared to a corresponding non-transformed wild type non-human organism.

Accordingly, in one embodiment, an increase of cryptoxanthin in a non-human organism, as compared to a corresponding non-transformed wild type non-human organism, is conferred in the process of the invention, if the activity of a polypeptide showing the activity of a melibiose carrier protein, or if the activity of the polypeptide B4120, preferably represented by SEQ ID NO. 130674, or a homolog or fragment thereof, or if the activity of a polypeptide encoded by a nucleic acid molecule comprising the nucleic acid SEQ ID NO. 130673, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 130673 or polypeptide SEQ ID NO. 130674, respectively, or a homolog or a fragment thereof, is increased or generated, or if the activity melibiose carrier protein is increased or generated in a non-human organism, like a microorganism or a plant cell, plant or part thereof, especially with non-targeted localization, whereby the respective line disclose in column 7 the fine chemical cryptoxanthin. For example, an increase of the cryptoxanthin of at least 1 percent, particularly in a range of 37 to 74-percent is conferred as compared to a corresponding non-transformed wild type non-human organism.

Accordingly, in one embodiment, an increase of zeaxanthin in a non-human organism, as compared to a corresponding non-transformed wild type non-human organism, is conferred in the process of the invention, if the activity of a polypeptide showing the activity of a melibiose carrier protein, or if the activity of the polypeptide B4120, preferably represented by SEQ ID NO. 130674, or a homolog or fragment thereof, or if the activity of a polypeptide encoded by a nucleic acid molecule comprising the nucleic acid SEQ ID NO. 130673, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 130673 or polypeptide SEQ ID NO. 130674, respectively, or a homolog or a fragment thereof, is increased or generated, or if the activity melibiose carrier protein is increased or generated in a non-human organism, like a microorganism or a plant cell, plant or part thereof, especially with non-targeted localization, whereby the respective line disclose in column 7 the fine chemical zeaxanthin. For example, an increase of the zeaxanthin of at least 1 percent, particularly in a range of 25 to 30-percent is conferred as compared to a corresponding non-transformed wild type non-human organism.

Accordingly, in one embodiment, an increase of violaxanthin in a non-human organism, as compared to a corresponding non-transformed wild type non-human organism, is conferred in the process of the invention, if the activity of a polypeptide showing the activity of a purinenucleoside phosphorylase, or if the activity of the polypeptide B4384, preferably represented by SEQ ID NO. 45557, or a homolog or fragment thereof, or if the activity of a polypeptide encoded by a nucleic acid molecule comprising the nucleic acid SEQ ID NO. 45556, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 45556 or polypeptide SEQ ID NO. 45557, respectively, or a homolog or a fragment thereof, is increased or generated, or if the activity purine-nucleoside phosphorylase is increased or generated in a non-human organism, like a microorganism or a plant cell, plant or part thereof, especially with non-targeted localization, whereby the respective line disclose in column 7 the fine chemical violaxanthin. For example, an increase of the violaxanthin of at least 1 percent, particularly in a range of 92 to 324-percent is conferred as compared to a corresponding non-transformed wild type non-human organism.

Accordingly, in one embodiment, an increase of zeaxanthin in a non-human organism, as compared to a corresponding non-transformed wild type non-human organism, is conferred in the process of the invention, if the activity of a polypeptide showing the activity of a transcription factor, or if the activity of the polypeptide GM02LC19289, preferably represented by SEQ ID NO. 46752, or a homolog or fragment thereof, or if the activity of a polypeptide encoded by a nucleic acid molecule comprising the nucleic acid SEQ ID NO. 46751, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 46751 or polypeptide SEQ ID NO. 46752, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical zeaxanthin. For example, an increase of the zeaxanthin of at least 1 percent, particularly in a range of 33 to 36-percent is conferred as compared to a corresponding non-transformed wild type non-human organism.

Accordingly, in one embodiment, an increase of cryptoxanthin in a non-human organism, as compared to a corresponding non-transformed wild type non-human organism, is conferred in the process of the invention, if the activity of a polypeptide showing the activity of a coproporganism phyrinogen oxidase, or if the activity of the polypeptide Sll1185, preferably represented by SEQ ID NO. 53190, or a homolog or fragment thereof, or if the activity of a polypeptide encoded by a nucleic acid molecule comprising the nucleic acid SEQ ID NO. 53189, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 53189 or polypeptide SEQ ID NO. 53190, respectively, or a homolog or a fragment thereof, is increased or generated, or if the activity coproporphyrinogen oxidase is increased or generated in a non-human organism, like a microorganism or a plant cell, plant or part thereof, especially with non-targeted localization, whereby the respective line disclose in column 7 the fine chemical cryptoxanthin. For example, an increase of the cryptoxanthin of at least 1 percent, particularly in a range of 35 to 47-percent is conferred as compared to a corresponding non-transformed wild type non-human organism.

Accordingly, in one embodiment, an increase of zeaxanthin in a non-human organism, as compared to a corresponding non-transformed wild type non-human organism, is conferred in the process of the invention, if the activity of a polypeptide showing the activity of a coproporphyrinogen oxidase, or if the activity of the polypeptide Sll1185, preferably represented by SEQ ID NO. 53190, or a homolog or fragment thereof, or if the activity of a polypeptide encoded by a nucleic acid molecule comprising the nucleic acid SEQ ID NO. 53189, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 53189 or polypeptide SEQ ID NO. 53190, respectively, or a homolog or a fragment thereof, is increased or generated, or if the activity coproporphyrinogen oxidase is increased or generated in a non-human organism, like a microorganism or a plant cell, plant or part thereof, especially with non-targeted localization, whereby the respective line disclose in column 7 the fine chemical zeaxanthin. For example, an increase of the zeaxanthin of at least 1 percent, particularly in a range of 28 to 48-percent is conferred as compared to a corresponding non-transformed wild type non-human organism.

Accordingly, in one embodiment, an increase of zeaxanthin in a non-human organism, as compared to a corresponding non-transformed wild type non-human organism, is conferred in the process of the invention, if the activity of a polypeptide showing the activity of a glycerol-3-phosphate dehydrogenase, or if the activity of the polypeptide Slr1755, preferably represented by SEQ ID NO. 58824, or a homolog or fragment thereof, or if the activity of a polypeptide encoded by a nucleic acid molecule comprising the nucleic acid SEQ ID NO. 58823, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 58823 or polypeptide SEQ ID NO. 58824, respectively, or a homolog or a fragment thereof, is increased or generated, or if the activity glycerol-3-phosphate dehydrogenase is increased or generated in a non-human organism, like a microorganism or a plant cell, plant or part thereof, especially with plastidic localization, whereby the respective line disclose in column 7 the fine chemical zeaxanthin. For example, an increase of the zeaxanthin of at least 1 percent, particularly in a range of 27 to 46-percent is conferred as compared to a corresponding non-transformed wild type non-human organism.

Accordingly, in one embodiment, an increase of cryptoxanthin in a non-human organism, as compared to a corresponding non-transformed wild type non-human organism, is conferred in the process of the invention, if the activity of a polypeptide showing the activity of a YCL026C-A-protein, or if the activity of the polypeptide Ycl026c-a, preferably represented by SEQ ID NO.

63168, or a homolog or fragment thereof, or if the activity of a polypeptide encoded by a nucleic acid molecule comprising the nucleic acid SEQ ID NO. 63167, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 63167 or polypeptide SEQ ID NO. 63168, respectively, or a homolog or a fragment thereof, is increased or generated, or if the activity YCL026C-A-protein is increased or generated in a non-human organism, like a microorganism or a plant cell, plant or part thereof, especially with non-targeted localization, whereby the respective line disclose in column 7 the fine chemical cryptoxanthin. For example, an increase of the cryptoxanthin of at least 1 percent, particularly in a range of 32 to 79-percent is conferred as compared to a corresponding non-transformed wild type non-human organism.

Accordingly, in one embodiment, an increase of cryptoxanthin in a non-human organism, as compared to a corresponding non-transformed wild type non-human organism, is conferred in the process of the invention, if the activity of a polypeptide showing the activity of a F-box protein, or if the activity of the polypeptide Ydr131c, preferably represented by SEQ ID NO. 14707, or a homolog or fragment thereof, or if the activity of a polypeptide encoded by a nucleic acid molecule comprising the nucleic acid SEQ ID NO. 14706, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 14706 or polypeptide SEQ ID NO. 14707, respectively, or a homolog or a fragment thereof, is increased or generated, or if the activity F-box protein is increased or generated in a non-human organism, like a microorganism or a plant cell, plant or part thereof, especially with plastidic localization, whereby the respective line disclose in column 7 the fine chemical cryptoxanthin. For example, an increase of the cryptoxanthin of at least 1 percent, particularly in a range of 38 to 133-percent is conferred as compared to a corresponding non-transformed wild type non-human organism.

Accordingly, in one embodiment, an increase of cryptoxanthin in a non-human organism, as compared to a corresponding non-transformed wild type non-human organism, is conferred in the process of the invention, if the activity of a polypeptide showing the activity of a ygl096w-protein, or if the activity of the polypeptide Ygl096w, preferably represented by SEQ ID NO. 90279, or a homolog or fragment thereof, or if the activity of a polypeptide encoded by a nucleic acid molecule comprising the nucleic acid SEQ ID NO. 90278, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table 1, 11 or IV, application No. 13, column 5 or 8 in the respective same line as the nucleic acid molecule SEQ ID NO. 90278 or polypeptide SEQ ID NO. 90279, respectively, or a homolog or a fragment thereof, is increased or generated, or if the activity ygl096w-protein is increased or generated in a non-human organism, like a microorganism or a plant cell, plant or part thereof, especially with plastidic localization, whereby the respective line disclose in column 7 the fine chemical cryptoxanthin. For example, an increase of the cryptoxanthin of at least 1 percent, particularly in a range of 39 to 59-percent is conferred as compared to a corresponding non-transformed wild type non-human organism.

Accordingly, in one embodiment, an increase of cryptoxanthin in a non-human organism, as compared to a corresponding non-transformed wild type non-human organism, is conferred in the process of the invention, if the activity of a polypeptide showing the activity of a alcohol dehydrogenase, or if the activity of the polypeptide Ygl256w, preferably represented by SEQ ID NO. 130743, or a homolog or fragment thereof, or if the activity of a polypeptide encoded by a nucleic acid molecule comprising the nucleic acid SEQ ID NO. 130742, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 130742 or polypeptide SEQ ID NO. 130743, respectively, or a homolog or a fragment thereof, is increased or generated, or if the activity alcohol dehydrogenase is increased or generated in a non-human organism, like a microorganism or a plant cell, plant or part thereof, especially with plastidic localization, whereby the respective line disclose in column 7 the fine chemical cryptoxanthin. For example, an increase of the cryptoxanthin of at least 1 percent, particularly in a range of 35 to 57-percent is conferred as compared to a corresponding non-transformed wild type non-human organism.

Accordingly, in one embodiment, an increase of cryptoxanthin in a non-human organism, as compared to a corresponding non-transformed wild type non-human organism, is conferred in the process of the invention, if the activity of a polypeptide showing the activity of a halotolerance protein, or if the activity of the polypeptide Ykr072c, preferably represented by SEQ ID NO. 130904, or a homolog or fragment thereof, or if the activity of a polypeptide encoded by a nucleic acid molecule comprising the nucleic acid SEQ ID NO. 130903, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 130903 or polypeptide SEQ ID NO. 130904, respectively, or a homolog or a fragment thereof, is increased or generated, or if the activity halotolerance protein is increased or generated in a non-human organism, like a microorganism or a plant cell, plant or part thereof, especially with non-targeted localization, whereby the respective line disclose in column 7 the fine chemical cryptoxanthin. For example, an increase of the cryptoxanthin of at least 1 percent, particularly in a range of 50 to 76-percent is conferred as compared to a corresponding non-transformed wild type non-human organism.

Accordingly, in one embodiment, an increase of zeaxanthin in a non-human organism, as compared to a corresponding non-transformed wild type non-human organism, is conferred in the process of the invention, if the activity of a polypeptide showing the activity of a halotolerance protein, or if the activity of the polypeptide Ykr072c, preferably represented by SEQ ID NO. 130904, or a homolog or fragment thereof, or if the activity of a polypeptide encoded by a nucleic acid molecule comprising the nucleic acid SEQ ID NO. 130903, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 130903 or polypeptide SEQ ID NO. 130904, respectively, or a homolog or a fragment thereof, is increased or generated, or if the activity halotolerance protein is increased or generated in a non-human organism, like a microorganism or a plant cell, plant or part thereof, especially with non-targeted localization, whereby the respective line disclose in column 7 the fine chemical zeaxanthin. For example, an increase of the zeaxanthin of at least 1 percent, particularly in a range of 25 to 52-percent is conferred as compared to a corresponding non-transformed wild type non-human organism.

Accordingly, in one embodiment, an increase of zeaxanthin in a non-human organism, as compared to a corresponding non-transformed wild type non-human organism, is conferred in the process of the invention, if the activity of a polypeptide showing the activity of a N-acetylglucosaminylphosphatidylinositoldeacetylase, or if the activity of the polypeptide Ymr281w, preferably represented by SEQ ID NO. 130933, or a homolog or fragment thereof, or if the activity of a polypeptide encoded by a nucleic acid molecule comprising the nucleic acid SEQ ID NO. 130932, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 130932 or polypeptide SEQ ID NO. 130933, respectively, or a homolog or a fragment thereof, is increased or generated, or if the activity N-acetylglucosaminylphosphatidylinositoldeacetylase is increased or generated in a non-human organism, like a microorganism or a plant cell, plant or part thereof, especially with plastidic localization, whereby the respective line disclose in column 7 the fine chemical zeaxanthin. For example, an increase of the zeaxanthin of at least 1 percent, particularly in a range of 28 to 81-percent is conferred as compared to a corresponding non-transformed wild type non-human organism.

Accordingly, in one embodiment, an increase of cryptoxanthin in a non-human organism, as compared to a corresponding non-transformed wild type non-human organism, is conferred in the process of the invention, if the activity of a polypeptide showing the activity of a ymr324c-protein, or if the activity of the polypeptide Ymr324c, preferably represented by SEQ ID NO. 130987, or a homolog or fragment thereof, or if the activity of a polypeptide encoded by a nucleic acid molecule comprising the nucleic acid SEQ ID NO. 130986, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 130986 or polypeptide SEQ ID NO. 130987, respectively, or a homolog or a fragment thereof, is increased or generated, or if the activity ymr324c-protein is increased or generated in a non-human organism, like a microorganism or a plant cell, plant or part thereof, especially with plastidic localization, whereby the respective line disclose in column 7 the fine chemical cryptoxanthin. For example, an increase of the cryptoxanthin of at least 1 percent, particularly in a range of 31 to 49-percent is conferred as compared to a corresponding non-transformed wild type non-human organism.

Accordingly, in one embodiment, an increase of zeaxanthin in a non-human organism, as compared to a corresponding non-transformed wild type non-human organism, is conferred in the process of the invention, if the activity of a polypeptide showing the activity of a sterol O-acyltransferase, or if the activity of the polypeptide Ynr019w, preferably represented by SEQ ID NO. 67647, or a homolog or fragment thereof, or if the activity of a polypeptide encoded by a nucleic acid molecule comprising the nucleic acid SEQ ID NO. 67646, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 67646 or polypeptide SEQ ID NO. 67647, respectively, or a homolog or a fragment thereof, is increased or generated, or if the activity sterol O-acyltransferase is increased or generated in a non-human organism, like a microorganism or a plant cell, plant or part thereof, especially with plastidic localization, whereby the respective line disclose in column 7 the fine chemical zeaxanthin. For example, an increase of the zeaxanthin of at least 1 percent, particularly in a range of 33 to 49-percent is conferred as compared to a corresponding non-transformed wild type non-human organism.

Accordingly, in one embodiment, an increase of abscisic acid in a non-human organism, as compared to a corresponding non-transformed wild type non-human organism, is conferred in the process of the invention, if the activity of a polypeptide showing the activity of a YER184C-protein, or if the activity of the polypeptide Yer184c, preferably represented by SEQ ID NO. 130714, or a homolog or fragment thereof, or if the activity of a polypeptide encoded by a nuoleic acid molecule comprising the nucleic acid SEQ ID NO. 130713, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 130713 or polypeptide SEQ ID NO. 130714, respectively, or a homolog or a fragment thereof, is increased or generated, or if the activity YER184C-protein is increased or generated in a non-human organism, like a microorganism or a plant cell, plant or part thereof, especially with non-targeted localization, whereby the respective line disclose in column 7 the fine chemical abscisic acid. For example, an increase of the abscisic acid of at least 1 percent, particularly in a range of 31 to 114-percent is conferred as compared to a corresponding non-transformed wild type non-human organism.

Accordingly, in one embodiment, an increase of abscisic acid in a non-human organism, as compared to a corresponding non-transformed wild type non-human organism, is conferred in the process of the invention, if the activity of a polypeptide showing the activity of a YOR044W-protein, or if the activity of the polypeptide YOR044W, preferably represented by SEQ ID NO. 131000, or a homolog or fragment thereof, or if the activity of a polypeptide encoded by a nucleic acid molecule comprising the nucleic acid SEQ ID NO. 130999, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 130999 or polypeptide SEQ ID NO. 131000, respectively, or a homolog or a fragment thereof, is increased or generated, or if the activity YOR044W-protein is increased or generated in a non-human organism, like a microorganism or a plant cell, plant or part thereof, especially with non-targeted localization, whereby the respective line disclose in column 7 the fine chemical abscisic acid. For example, an increase of the abscisic acid of at least 1 percent, particularly in a range of 3 to 113-percent is conferred as compared to a corresponding non-transformed wild type non-human organism.

Accordingly, in one embodiment, an increase of abscisic acid in a non-human organism, as compared to a corresponding non-transformed wild type non-human organism, is conferred in the process of the invention, if the activity of a polypeptide showing the activity of a gluconate transport system permease 3, or if the activity of the polypeptide B4321, preferably represented by SEQ ID NO. 45395, or a homolog or fragment thereof, or if the activity of a polypeptide encoded by a nucleic acid molecule comprising the nucleic acid SEQ ID NO. 45394, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 45394 or polypeptide SEQ ID NO. 45395, respectively, or a homolog or a fragment thereof, is increased or generated, or if the activity gluconate transport system permease 3 is increased or generated in a non-human organism, like a microorganism or a plant cell, plant or part thereof, especially with non-targeted localization, whereby the respective line disclose in column 7 the fine chemical abscisic acid. For example, an increase of the abscisic acid of at least 1 percent, particularly in a range of 25 to 283-percent is conferred as compared to a corresponding non-transformed wild type non-human organism.

Accordingly, in one embodiment, an increase of abscisic acid in a non-human organism, as compared to a corresponding non-transformed wild type non-human organism, is conferred in the process of the invention, if the activity of a polypeptide showing the activity of a nitrate/nitrite transport protein, or if the activity of the polypeptide Sll1450, preferably represented by SEQ ID NO. 54338, or a homolog or fragment thereof, or if the activity of a polypeptide encoded by a nucleic acid molecule comprising the nucleic acid SEQ ID NO. 54337, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table 1, 11 or IV, application No. 13, column 5 or 8 in the respective same line as the nucleic acid molecule SEQ ID NO. 54337 or polypeptide SEQ ID NO. 54338, respectively, or a homolog or a fragment thereof, is increased or generated, or if the activity nitrate/nitrite transport protein is increased or generated in a non-human organism, like a microorganism or a plant cell, plant or part thereof, especially with mitochondrial localization, whereby the respective line disclose in column 7 the fine chemical abscisic acid. For example, an increase of the abscisic acid of at least 1 percent, particularly in a range of 22 to 115-percent is conferred as compared to a corresponding non-transformed wild type non-human organism.

Accordingly, in one embodiment, an increase of abscisic acid in a non-human organism, as compared to a corresponding non-transformed wild type non-human organism, is conferred in the process of the invention, if the activity of a polypeptide showing the activity of a acyl transferase, or if the activity of the polypeptide Sll1848, preferably represented by SEQ ID NO. 86448, or a homolog or fragment thereof, or if the activity of a polypeptide encoded by a nucleic acid molecule comprising the nucleic acid SEQ ID NO. 86447, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 86447 or polypeptide SEQ ID NO. 86448, respectively, or a homolog or a fragment thereof, is increased or generated, or if the activity acyl transferase is increased or generated in a non-human organism, like a microorganism or a plant cell, plant or part thereof, especially with plastidic localization, whereby the respective line disclose in column 7 the fine chemical abscisic acid. For example, an increase of the abscisic acid of at least 1 percent, particularly in a range of 26 to 364-percent is conferred as compared to a corresponding non-transformed wild type non-human organism.

Accordingly, in one embodiment, an increase of abscisic acid in a non-human organism, as compared to a corresponding non-transformed wild type non-human organism, is conferred in the process of the invention, if the activity of a polypeptide showing the activity of a heat shock transcription factor, or if the activity of the polypeptide At3g02990, preferably represented by SEQ ID NO. 68658, or a homolog or fragment thereof, or if the activity of a polypeptide encoded by a nucleic acid molecule comprising the nucleic acid SEQ ID NO. 68657, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 68657 or polypeptide SEQ ID NO. 68658, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line disclose in column 7 the fine chemical abscisic acid. For example, an increase of the abscisic acid of at least 1 percent, particularly in a range of 14 to 231-percent is conferred as compared to a corresponding non-transformed wild type non-human organism.

Accordingly, in one embodiment, an increase of abscisic acid in a non-human organism, as compared to a corresponding non-transformed wild type non-human organism, is conferred in the process of the invention, if the activity of a polypeptide showing the activity of a protein kinase, or if the activity of the polypeptide At1 g48260, preferably represented by SEQ ID NO. 18236, or a homolog or fragment thereof, or if the activity of a polypeptide encoded by a nucleic acid molecule comprising the nucleic acid SEQ ID NO. 18235, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 18235 or polypeptide SEQ ID NO. 18236, respectively, or a homolog or a fragment thereof, is increased or generated, or if the activity protein kinase is increased or generated in a non-human organism, like a microorganism or a plant cell, plant or part thereof, especially with non-targeted localization, whereby the respective line disclose in column 7 the fine chemical abscisic acid. For example, an increase of the abscisic acid of at least 1 percent, particularly in a range of 29 to 300-percent is conferred as compared to a corresponding non-transformed wild type non-human organism.

Accordingly, in one embodiment, an increase of abscisic acid in a non-human organism, as compared to a corresponding non-transformed wild type non-human organism, is conferred in the process of the invention, if the activity of a polypeptide showing the activity of a protein phosphatase, or if the activity of the polypeptide At1g72770, preferably represented by SEQ ID NO. 19420, or a homolog or fragment thereof, or if the activity of a polypeptide encoded by a nucleic acid molecule comprising the nucleic acid SEQ ID NO. 19419, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 19419 or polypeptide SEQ ID NO. 19420, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical abscisic acid. For example, an increase of the abscisic acid of at least 1 percent, particularly in a range of 124 to 1187-percent is conferred as compared to a corresponding non-transformed wild type non-human organism.

Accordingly, in one embodiment, an increase of abscisic acid in a non-human organism, as compared to a corresponding non-transformed wild type non-human organism, is conferred in the process of the invention, if the activity of a polypeptide showing the activity of a glycine cleavage system T aminomethyltransferase, or if the activity of the polypeptide AVINDRAFT_{—}0539, preferably represented by SEQ ID NO. 70716, or a homolog or fragment thereof, or if the activity of a polypeptide encoded by a nucleic acid molecule comprising the nucleic acid SEQ ID NO. 70715, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 70715 or polypeptide SEQ ID NO. 70716, respectively, or a homolog or a fragment thereof, is increased or generated, or if the activity glycine cleavage system T aminomethyltransferase is increased or generated in a non-human organism, like a microorganism or a plant cell, plant or part thereof, especially with non-targeted localization, whereby the respective line disclose in column 7 the fine chemical abscisic acid. For example, an increase of the abscisic acid of at least 1 percent, particularly in a range of 12 to 162-percent is conferred as compared to a corresponding non-transformed wild type non-human organism.

Accordingly, in one embodiment, an increase of abscisic acid in a non-human organism, as compared to a corresponding non-transformed wild type non-human organism, is conferred in the process of the invention, if the activity of a polypeptide showing the activity of a glutaredoxin, or if the activity of the polypeptide At2g30540, preferably represented by SEQ ID NO. 20347, or a homolog or fragment thereof, or if the activity of a polypeptide encoded by a nucleic acid molecule comprising the nucleic acid SEQ ID NO. 20346, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 13, column 5 or 8 in the respective same line as the nucleic acid molecule SEQ ID NO. 20346 or polypeptide SEQ ID NO. 20347, respectively, or a homolog or a fragment thereof, is increased or generated, or if the activity glutaredoxin is increased or generated in a non-human organism, like a microorganism organism or a plant cell, plant or part thereof, especially with non-targeted localization, whereby the respective line disclose in column 7 the fine chemical abscisic acid. For example, an increase of the abscisic acid of at least 1 percent, particularly in a range of 11 to 152-percent is conferred as compared to a corresponding non-transformed wild type non-human organism.

[0096.1.7.13] to [0103.1.7.13] for the disclosure of these paragraphs see [0096.1.7.7] to [0103.1.7.7] above

In this paragraph “A” means “Goffeau et al., Science 274 (5287), 546 (1996)”, “B” means “Blattner et al., Science 277 (5331), 1453 (1997)”, “C” means “Nakamura et al., Nucleic Acids Res. 28(1), 72 (2000)”, “D” means “deposition of the sequences to the EMBL/GenBank/DDBJ databases under “Sequencing of the draft genome assembly of Azotobacter vinelandii AvOP”. in June 2005 by Copeland et al.”, “E” means “Henne et al., Nat. Biotechnol. 22 (5), 547 (2004)”, “F” means “Kaul et al., Nature 408 (6814), 796 (2000)”, and “G” means “Matsumoto et al., Nature 436 (7052), 793 (2005)”.

The nucleic acid sequence of At3g15360 from Arabidopsis thaliana, e.g. as shown in the respective line in column 5 of Table I, application no. 13, has been published in F. And the activity of the gene product thereof is the activity of thioredoxin.

Accordingly, in one embodiment, the process of the present invention for producing cryptoxanthin in a non-human organism, like a microorganism or a plant or a part thereof, comprises increasing or generating the activity of a gene product with the activity of a gene product conferring the activity of “thioredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical cryptoxanthin), application no. 13, preferably the coding region thereof, or a homolog or a fragment thereof, and being depicted in the same respective line as said At3g15360, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said At3g15360, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said At3g15360, or a functional equivalent or a homolog thereof as depicted in column 8 of Table II, application no. 13, preferably a homolog or functional equivalent as depicted in column 8 of Table II B, application no. 13, and being depicted in the same respective line as said At3g15360, and preferably the activity is increased non-targeted, whereby the respective line disclose in column 7 the fine chemical cryptoxanthin.

Accordingly, in one embodiment, the molecule which activity is to be increased in the process of the invention is the gene product with an activity as a “thioredoxin”, preferably it is the molecule of section (a) or (b) of this paragraph.

In particular, it was observed that in plants, especially in Arabidopsis thaliana, increasing or generating the activity of a gene product non-targeted with the activity of a “thioredoxin”, preferably being encoded by a gene comprising the nucleic acid sequence SEQ ID NO. 130491, preferably the coding region thereof, conferred the production of or the increase in cryptoxanthin compared with the wild type control.

The nucleic acid sequence of At3g16820 from Arabidopsis thaliana, e.g. as shown in the respective line in column 5 of Table I, application no. 13, has been published in F. And the activity of the gene product thereof is the activity of F-box protein.

Accordingly, in one embodiment, the process of the present invention for producing zeaxanthin in a non-human organism, like a microorganism or a plant or a part thereof, comprises increasing or generating the activity of a gene product with the activity of a gene product conferring the activity of “F-box protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical zeaxanthin), application no. 13, preferably the coding region thereof, or a homolog or a fragment thereof, and being depicted in the same respective line as said At3g16820, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said At3g16820, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said At3g16820, or a functional equivalent or a homolog thereof as depicted in column 8 of Table II, application no. 13, preferably a homolog or functional equivalent as depicted in column 8 of Table II B, application no. 13, and being depicted in the same respective line as said At3g16820, and preferably the activity is increased non-targeted, whereby the respective line disclose in column 7 the fine chemical zeaxanthin.

Accordingly, in one embodiment, the molecule which activity is to be increased in the process of the invention is the gene product with an activity as a “F-box protein”, preferably it is the molecule of section (a) or (b) of this paragraph.

In particular, it was observed that in plants, especially in Arabidopsis thaliana, increasing or generating the activity of a gene product non-targeted with the activity of a “F-box protein”, preferably being encoded by a gene comprising the nucleic acid sequence SEQ ID NO. 120952, preferably the coding region thereof, conferred the production of or the increase in zeaxanthin compared with the wild type control.

The nucleic acid sequence of At3g62930 from Arabidopsis thaliana, e.g. as shown in the respective line in column 5 of Table I, application no. 13, has been published in F. And the activity of the gene product thereof is the activity of monthiol glutaredoxin.

Accordingly, in one embodiment, the process of the present invention for producing zeaxanthin in a non-human organism, like a microorganism or a plant or a part thereof, comprises increasing or generating the activity of a gene product with the activity of a gene product conferring the activity of “monthiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical zeaxanthin), application no. 13, preferably the coding region thereof, or a homolog or a fragment thereof, and being depicted in the same respective line as said At3g62930, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said At3g62930, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said At3g62930, or a functional equivalent or a homolog thereof as depicted in column 8 of Table II, application no. 13, preferably a homolog or functional equivalent as depicted in column 8 of Table II B, application no. 13, and being depicted in the same respective line as said At3g62930, and preferably the activity is increased non-targeted, whereby the respective line disclose in column 7 the fine chemical zeaxanthin.

Accordingly, in one embodiment, the molecule which activity is to be increased in the process of the invention is the gene product with an activity as a “monthiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monthiol glutaredoxin”, preferably being encoded by a gene comprising the nucleic acid sequence SEQ ID NO. 2573, preferably the coding region thereof, conferred the production of or the increase in zeaxanthin compared with the wild type control.

The nucleic acid sequence of At4g15660 from Arabidopsis thaliana, e.g. as shown in the respective line in column 5 of Table I, application no. 13, has been published in F. And the activity of the gene product thereof is the activity of monothiol glutaredoxin.

Accordingly, in one embodiment, the process of the present invention for producing zeaxanthin in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical zeaxanthin), application no. 13, preferably the coding region thereof, or a homolog or a fragment thereof, and being depicted in the same respective line as said At4g15660, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said At4g15660, or a functional equivalent or a homolog thereof as depicted in column 8 of Table II, application no. 13, preferably a homolog or functional equivalent as depicted in column 8 of Table II B, application no. 13, and being depicted in the same respective line as said At4g15660, and preferably the activity is increased non-targeted, whereby the respective line disclose in column 7 the fine chemical zeaxanthin.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in zeaxanthin compared with the wild type control.

The nucleic acid sequence of At4g15670 from Arabidopsis thaliana, e.g. as shown in the respective line in column 5 of Table I, application no. 13, has been published in F. And the activity of the gene product thereof is the activity of monothiol glutaredoxin.

Accordingly, in one embodiment, the process of the present invention for producing zeaxanthin in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical zeaxanthin), application no. 13, preferably the coding region thereof, or a homolog or a fragment thereof, and being depicted in the same respective line as said At4g15670, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said At4g15670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said At4g15670, or a functional equivalent or a homolog thereof as depicted in column 8 of Table II, application no. 13, preferably a homolog or functional equivalent as depicted in column 8 of Table II B, application no. 13, and being depicted in the same respective line as said At4g15670, and preferably the activity is increased non-targeted, whereby the respective line disclose in column 7 the fine chemical zeaxanthin.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3279, preferably the coding region thereof, conferred the production of or the increase in zeaxanthin compared with the wild type control.

The nucleic acid sequence of At4g26080 from Arabidopsis thaliana, e.g. as shown in the respective line in column 5 of Table I, application no. 13, has been published in F. And the activity of the gene product thereof is the activity of protein phosphatase.

Accordingly, in one embodiment, the process of the present invention for producing zeaxanthin in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical zeaxanthin), application no. 13, preferably the coding region thereof, or a homolog or a fragment thereof, and being depicted in the same respective line as said At4g26080, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said At4g26080, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said At4g26080, or a functional equivalent or a homolog thereof as depicted in column 8 of Table II, application no. 13, preferably a homolog or functional equivalent as depicted in column 8 of Table II B, application no. 13, and being depicted in the same respective line as said At4g26080, and preferably the activity is increased non-targeted, whereby the respective line disclose in column 7 the fine chemical zeaxanthin.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 68849, preferably the coding region thereof, conferred the production of or the increase in zeaxanthin compared with the wild type control.

The nucleic acid sequence of At4g33040 from Arabidopsis thaliana, e.g. as shown in the respective line in column 5 of Table I, application no. 13, has been published in F. And the activity of the gene product thereof is the activity of glutaredoxin.

Accordingly, in one embodiment, the process of the present invention for producing cryptoxanthin in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical cryptoxanthin), application no. 13, preferably the coding region thereof, or a homolog or a fragment thereof, and being depicted in the same respective line as said At4g33040, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said At4g33040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said At4g33040, or a functional equivalent or a homolog thereof as depicted in column 8 of Table II, application no. 13, preferably a homolog or functional equivalent as depicted in column 8 of Table II B, application no. 13, and being depicted in the same respective line as said At4g33040, and preferably the activity is increased non-targeted, whereby the respective line disclose in column 7 the fine chemical cryptoxanthin.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 4102, preferably the coding region thereof, conferred the production of or the increase in cryptoxanthin compared with the wild type control.

The nucleic acid sequence of At5g60440 from Arabidopsis thaliana, e.g. as shown in the respective line in column 5 of Table I, application no. 13, has been published in F. And the activity of the gene product thereof is the activity of MADS-box transcription factor.

Accordingly, in one embodiment, the process of the present invention for producing zeaxanthin in a non-human organism, like a microorganism or a plant or a part thereof, comprises increasing or generating the activity of a gene product with the activity of a gene product conferring the activity of “MADS-box transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical zeaxanthin), application no. 13, preferably the coding region thereof, or a homolog or a fragment thereof, and being depicted in the same respective line as said At5g60440, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said At5g60440, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said At5g60440, or a functional equivalent or a homolog thereof as depicted in column 8 of Table II, application no. 13, preferably a homolog or functional equivalent as depicted in column 8 of Table II B, application no. 13, and being depicted in the same respective line as said At5g60440, and preferably the activity is increased non-targeted, whereby the respective line disclose in column 7 the fine chemical zeaxanthin.

Accordingly, in one embodiment, the molecule which activity is to be increased in the process of the invention is the gene product with an activity as a “MADS-box transcription factor”, preferably it is the molecule of section (a) or (b) of this paragraph.

In particular, it was observed that in plants, especially in Arabidopsis thaliana, increasing or generating the activity of a gene product non-targeted with the activity of a “MADS-box transcription factor”, preferably being encoded by a gene comprising the nucleic acid sequence SEQ ID NO. 102899, preferably the coding region thereof, conferred the production of or the increase in zeaxanthin compared with the wild type control.

The nucleic acid sequence of AvinDRAFT_{—}4847 from Azotobacter vinelandii, e.g. as shown in the respective line in column 5 of Table I, application no. 13, has been published in D. And the activity of the gene product thereof is the activity of isopropylmalate isomerase large subunit.

Accordingly, in one embodiment, the process of the present invention for producing zeaxanthin in a non-human organism, like a microorganism or a plant or a part thereof, comprises increasing or generating the activity of a gene product with the activity of a gene product conferring the activity of “isopropylmalate isomerase large subunit”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical zeaxanthin), application no. 13, preferably the coding region thereof, or a homolog or a fragment thereof, and being depicted in the same respective line as said AvinDRAFT_{—}4847, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said AvinDRAFT_{—}4847, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said AvinDRAFT_{—}4847, or a functional equivalent or a homolog thereof as depicted in column 8 of Table II, application no. 13, preferably a homolog or functional equivalent as depicted in column 8 of Table II B, application no. 13, and being depicted in the same respective line as said AvinDRAFT_{—}4847, and preferably the activity is increased non-targeted, whereby the respective line disclose in column 7 the fine chemical zeaxanthin.

Accordingly, in one embodiment, the molecule which activity is to be increased in the process of the invention is the gene product with an activity as a “isopropylmalate isomerase large subunit”, preferably it is the molecule of section (a) or (b) of this paragraph.

In particular, it was observed that in plants, especially in Arabidopsis thaliana, increasing or generating the activity of a gene product non-targeted with the activity of a “isopropylmalate isomerase large subunit”, preferably being encoded by a gene comprising the nucleic acid sequence SEQ ID NO. 102941, preferably the coding region thereof, conferred the production of or the increase in zeaxanthin compared with the wild type control.

The nucleic acid sequence of AX653549 from Oryza sativa, e.g. as shown in the respective line in column 5 of Table I, application no. 13, has been published in G. And the activity of the gene product thereof is the activity of AX653549-protein.

Accordingly, in one embodiment, the process of the present invention for producing cryptoxanthin in a non-human organism, like a microorganism or a plant or a part thereof, comprises increasing or generating the activity of a gene product with the activity of a gene product conferring the activity of “AX653549-protein”, especially from Oryza sativa or its functional equivalent or its homolog, e.g. the increase of
(a) a gene product of a gene comprising the nucleic acid molecule as shown in the respective line in column 5 of Table I (whereby the respective line disclose in column 7 the fine chemical cryptoxanthin), application no. 13, preferably the coding region thereof, or a homolog or a fragment thereof, and being depicted in the same respective line as said AX653549, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said AX653549, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said AX653549, or a functional equivalent or a homolog thereof as depicted in column 8 of Table II, application no. 13, preferably a homolog or functional equivalent as depicted in column 8 of Table II B, application no. 13, and being depicted in the same respective line as said AX653549, and preferably the activity is increased non-targeted, whereby the respective line disclose in column 7 the fine chemical cryptoxanthin.

Accordingly, in one embodiment, the molecule which activity is to be increased in the process of the invention is the gene product with an activity as a “AX653549-protein”, preferably it is the molecule of section (a) or (b) of this paragraph.

In particular, it was observed that in plants, especially in Arabidopsis thaliana, increasing or generating the activity of a gene product non-targeted with the activity of a “AX653549-protein”, preferably being encoded by a gene comprising the nucleic acid sequence SEQ ID NO. 34301, preferably the coding region thereof, conferred the production of or the increase in cryptoxanthin compared with the wild type control.

The nucleic acid sequence of B1003 from Escherichia coli, e.g. as shown in the respective line in column 5 of Table I, application no. 13, has been published in B. And the activity of the gene product thereof is the activity of b1003-protein.

Accordingly, in one embodiment, the process of the present invention for producing zeaxanthin in a non-human organism, like a microorganism or a plant or a part thereof, comprises increasing or generating the activity of a gene product with the activity of a gene product conferring the activity of “b1003-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical zeaxanthin), application no. 13, preferably the coding region thereof, or a homolog or a fragment thereof, and being depicted in the same respective line as said B1003, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said B1003, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said B1003, or a functional equivalent or a homolog thereof as depicted in column 8 of Table II, application no. 13, preferably a homolog or functional equivalent as depicted in column 8 of Table II B, application no. 13, and being depicted in the same respective line as said B1003, and preferably the activity is increased non-targeted, whereby the respective line disclose in column 7 the fine chemical zeaxanthin.

Accordingly, in one embodiment, the molecule which activity is to be increased in the process of the invention is the gene product with an activity as a “b1003-protein”, preferably it is the molecule of section (a) or (b) of this paragraph.

In particular, it was observed that in plants, especially in Arabidopsis thaliana, increasing or generating the activity of a gene product non-targeted with the activity of a “b1003-protein”, preferably being encoded by a gene comprising the nucleic acid sequence SEQ ID NO. 7941, preferably the coding region thereof, conferred the production of or the increase in zeaxanthin compared with the wild type control.

The nucleic acid sequence of B1023 from Escherichia coli, e.g. as shown in the respective line in column 5 of Table I, application no. 13, has been published in B. And the activity of the gene product thereof is the activity of lipoprotein precursor.

Accordingly, in one embodiment, the process of the present invention for producing zeaxanthin in a non-human organism, like a microorganism or a plant or a part thereof, comprises increasing or generating the activity of a gene product with the activity of a gene product conferring the activity of “lipoprotein precursor”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical zeaxanthin), application no. 13, preferably the coding region thereof, or a homolog or a fragment thereof, and being depicted in the same respective line as said B1023, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said B1023, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said B1023, or a functional equivalent or a homolog thereof as depicted in column 8 of Table II, application no. 13, preferably a homolog or functional equivalent as depicted in column 8 of Table II B, application no. 13, and being depicted in the same respective line as said B1023, and preferably the activity is increased non-targeted, whereby the respective line disclose in column 7 the fine chemical zeaxanthin.

Accordingly, in one embodiment, the molecule which activity is to be increased in the process of the invention is the gene product with an activity as a “lipoprotein precursor”, preferably it is the molecule of section (a) or (b) of this paragraph.

In particular, it was observed that in plants, especially in Arabidopsis thaliana, increasing or generating the activity of a gene product non-targeted with the activity of a “lipoprotein precursor”, preferably being encoded by a gene comprising the nucleic acid sequence SEQ ID NO. 36880, preferably the coding region thereof, conferred the production of or the increase in zeaxanthin compared with the wild type control.

The nucleic acid sequence of B1838 from Escherichia coli, e.g. as shown in the respective line in column 5 of Table I, application no. 13, has been published in B. And the activity of the gene product thereof is the activity of serine/threonine-protein phosphatase.

Accordingly, in one embodiment, the process of the present invention for producing zeaxanthin in a non-human organism, like a microorganism or a plant or a part thereof, comprises increasing or generating the activity of a gene product with the activity of a gene product conferring the activity of “serine/threonine-protein phosphatase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical zeaxanthin), application no. 13, preferably the coding region thereof, or a homolog or a fragment thereof, and being depicted in the same respective line as said B1838, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said B1838, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said B1838, or a functional equivalent or a homolog thereof as depicted in column 8 of Table II, application no. 13, preferably a homolog or functional equivalent as depicted in column 8 of Table II B, application no. 13, and being depicted in the same respective line as said B1838, and preferably the activity is increased non-targeted, whereby the respective line disclose in column 7 the fine chemical zeaxanthin.

Accordingly, in one embodiment, the molecule which activity is to be increased in the process of the invention is the gene product with an activity as a “serine/threonine-protein phosphatase”, preferably it is the molecule of section (a) or (b) of this paragraph.

In particular, it was observed that in plants, especially in Arabidopsis thaliana, increasing or generating the activity of a gene product non-targeted with the activity of a “serine/threonine-protein phosphatase”, preferably being encoded by a gene comprising the nucleic acid sequence SEQ ID NO. 8316, preferably the coding region thereof, conferred the production of or the increase in zeaxanthin compared with the wild type control.

The nucleic acid sequence of B2032 from Escherichia coli, e.g. as shown in the respective line in column 5 of Table I, application no. 13, has been published in B. And the activity of the gene product thereof is the activity of b2032-protein.

Accordingly, in one embodiment, the process of the present invention for producing zeaxanthin in a non-human organism, like a microorganism or a plant or a part thereof, comprises increasing or generating the activity of a gene product with the activity of a gene product conferring the activity of “b2032-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical zeaxanthin), application no. 13, preferably the coding region thereof, or a homolog or a fragment thereof, and being depicted in the same respective line as said B2032, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said B2032, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said B2032, or a functional equivalent or a homolog thereof as depicted in column 8 of Table II, application no. 13, preferably a homolog or functional equivalent as depicted in column 8 of Table II B, application no. 13, and being depicted in the same respective line as said B2032, and preferably the activity is increased non-targeted, whereby the respective line disclose in column 7 the fine chemical zeaxanthin.

Accordingly, in one embodiment, the molecule which activity is to be increased in the process of the invention is the gene product with an activity as a “b2032-protein”, preferably it is the molecule of section (a) or (b) of this paragraph.

In particular, it was observed that in plants, especially in Arabidopsis thaliana, increasing or generating the activity of a gene product non-targeted with the activity of a “b2032-protein”, preferably being encoded by a gene comprising the nucleic acid sequence SEQ ID NO. 8920, preferably the coding region thereof, conferred the production of or the increase in zeaxanthin compared with the wild type control.

The nucleic acid sequence of B2099 from Escherichia coli, e.g. as shown in the respective line in column 5 of Table I, application no. 13, has been published in B. And the activity of the gene product thereof is the activity of b2099-protein.

Accordingly, in one embodiment, the process of the present invention for producing cryptoxanthin in a non-human organism, like a microorganism or a plant or a part thereof, comprises increasing or generating the activity of a gene product with the activity of a gene product conferring the activity of “b2099-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical cryptoxanthin), application no. 13, preferably the coding region thereof, or a homolog or a fragment thereof, and being depicted in the same respective line as said B2099, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said B2099, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said B2099, or a functional equivalent or a homolog thereof as depicted in column 8 of Table II, application no. 13, preferably a homolog or functional equivalent as depicted in column 8 of Table II B, application no. 13, and being depicted in the same respective line as said B2099, and preferably the activity is increased non-targeted, whereby the respective line disclose in column 7 the fine chemical cryptoxanthin.

Accordingly, in one embodiment, the molecule which activity is to be increased in the process of the invention is the gene product with an activity as a “b2099-protein”, preferably it is the molecule of section (a) or (b) of this paragraph.

In particular, it was observed that in plants, especially in Arabidopsis thaliana, increasing or generating the activity of a gene product non-targeted with the activity of a “b2099-protein”, preferably being encoded by a gene comprising the nucleic acid sequence SEQ ID NO. 115595, preferably the coding region thereof, conferred the production of or the increase in cryptoxanthin compared with the wild type control.

The nucleic acid sequence of B2121 from Escherichia coli, e.g. as shown in the respective line in column 5 of Table I, application no. 13, has been published in B. And the activity of the gene product thereof is the activity of b2121-protein.

Accordingly, in one embodiment, the process of the present invention for producing zeaxanthin in a non-human organism, like a microorganism or a plant or a part thereof, comprises increasing or generating the activity of a gene product with the activity of a gene product conferring the activity of “b2121-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical zeaxanthin), application no. 13, preferably the coding region thereof, or a homolog or a fragment thereof, and being depicted in the same respective line as said B2121, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said B2121, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said B2121, or a functional equivalent or a homolog thereof as depicted in column 8 of Table II, application no. 13, preferably a homolog or functional equivalent as depicted in column 8 of Table II B, application no. 13, and being depicted in the same respective line as said B2121, and preferably the activity is increased non-targeted, whereby the respective line disclose in column 7 the fine chemical zeaxanthin.

Accordingly, in one embodiment, the molecule which activity is to be increased in the process of the invention is the gene product with an activity as a “b2121-protein”, preferably it is the molecule of section (a) or (b) of this paragraph.

In particular, it was observed that in plants, especially in Arabidopsis thaliana, increasing or generating the activity of a gene product non-targeted with the activity of a “b2121-protein”, preferably being encoded by a gene comprising the nucleic acid sequence SEQ ID NO. 39013, preferably the coding region thereof, conferred the production of or the increase in zeaxanthin compared with the wild type control.

The nucleic acid sequence of B2474 from Escherichia coli, e.g. as shown in the respective line in column 5 of Table I, application no. 13, has been published in B. And the activity of the gene product thereof is the activity of b2474-protein.

Accordingly, in one embodiment, the process of the present invention for producing zeaxanthin in a non-human organism, like a microorganism or a plant or a part thereof, comprises increasing or generating the activity of a gene product with the activity of a gene product conferring the activity of “b2474-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical zeaxanthin), application no. 13, preferably the coding region thereof, or a homolog or a fragment thereof, and being depicted in the same respective line as said B2474, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said B2474, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said B2474, or a functional equivalent or a homolog thereof as depicted in column 8 of Table II, application no. 13, preferably a homolog or functional equivalent as depicted in column 8 of Table II B, application no. 13, and being depicted in the same respective line as said B2474, and preferably the activity is increased non-targeted, whereby the respective line disclose in column 7 the fine chemical zeaxanthin.

Accordingly, in one embodiment, the molecule which activity is to be increased in the process of the invention is the gene product with an activity as a “b2474-protein”, preferably it is the molecule of section (a) or (b) of this paragraph.

In particular, it was observed that in plants, especially in Arabidopsis thaliana, increasing or generating the activity of a gene product non-targeted with the activity of a “b2474-protein”, preferably being encoded by a gene comprising the nucleic acid sequence SEQ ID NO. 40329, preferably the coding region thereof, conferred the production of or the increase in zeaxanthin compared with the wild type control.

The nucleic acid sequence of B2714 from Escherichia coli, e.g. as shown in the respective line in column 5 of Table I, application no. 13, has been published in B. And the activity of the gene product thereof is the activity of transcriptional regulator.

Accordingly, in one embodiment, the process of the present invention for producing zeaxanthin in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical zeaxanthin), application no. 13, preferably the coding region thereof, or a homolog or a fragment thereof, and being depicted in the same respective line as said B2714, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said B2714, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said B2714, or a functional equivalent or a homolog thereof as depicted in column 8 of Table II, application no. 13, preferably a homolog or functional equivalent as depicted in column 8 of Table II B, application no. 13, and being depicted in the same respective line as said B2714, and preferably the activity is increased non-targeted, whereby the respective line disclose in column 7 the fine chemical zeaxanthin.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 40741, preferably the coding region thereof, conferred the production of or the increase in zeaxanthin compared with the wild type control.

The nucleic acid sequence of B2790 from Escherichia coli, e.g. as shown in the respective line in column 5 of Table I, application no. 13, has been published in B. And the activity of the gene product thereof is the activity of flavodoxin.

Accordingly, in one embodiment, the process of the present invention for producing cryptoxanthin in a non-human organism, like a microorganism or a plant or a part thereof, comprises increasing or generating the activity of a gene product with the activity of a gene product conferring the activity of “flavodoxin”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical cryptoxanthin), application no. 13, preferably the coding region thereof, or a homolog or a fragment thereof, and being depicted in the same respective line as said B2790, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said B2790, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said B2790, or a functional equivalent or a homolog thereof as depicted in column 8 of Table II, application no. 13, preferably a homolog or functional equivalent as depicted in column 8 of Table II B, application no. 13, and being depicted in the same respective line as said B2790, and preferably the activity is increased non-targeted, whereby the respective line disclose in column 7 the fine chemical cryptoxanthin.

Accordingly, in one embodiment, the molecule which activity is to be increased in the process of the invention is the gene product with an activity as a “flavodoxin”, preferably it is the molecule of section (a) or (b) of this paragraph.

In particular, it was observed that in plants, especially in Arabidopsis thaliana, increasing or generating the activity of a gene product non-targeted with the activity of a “flavodoxin”, preferably being encoded by a gene comprising the nucleic acid sequence SEQ ID NO. 115719, preferably the coding region thereof, conferred the production of or the increase in cryptoxanthin compared with the wild type control.

The nucleic acid sequence of B2914 from Escherichia coli, e.g. as shown in the respective line in column 5 of Table I, application no. 13, has been published in B. And the activity of the gene product thereof is the activity of ribosephosphate isomerase, constitutive.

Accordingly, in one embodiment, the process of the present invention for producing violaxanthin in a non-human organism, like a microorganism or a plant or a part thereof, comprises increasing or generating the activity of a gene product with the activity of a gene product conferring the activity of “ribosephosphate isomerase, constitutive”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical violaxanthin), application no. 13, preferably the coding region thereof, or a homolog or a fragment thereof, and being depicted in the same respective line as said B2914, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said B2914, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said B2914, or a functional equivalent or a homolog thereof as depicted in column 8 of Table II, application no. 13, preferably a homolog or functional equivalent as depicted in column 8 of Table II B, application no. 13, and being depicted in the same respective line as said B2914, and preferably the activity is increased non-targeted, whereby the respective line disclose in column 7 the fine chemical violaxanthin.

Accordingly, in one embodiment, the molecule which activity is to be increased in the process of the invention is the gene product with an activity as a “ribosephosphate isomerase, constitutive”, preferably it is the molecule of section (a) or (b) of this paragraph.

In particular, it was observed that in plants, especially in Arabidopsis thaliana, increasing or generating the activity of a gene product non-targeted with the activity of a “ribosephosphate isomerase, constitutive”, preferably being encoded by a gene comprising the nucleic acid sequence SEQ ID NO. 41073, preferably the coding region thereof, conferred the production of or the increase in violaxanthin compared with the wild type control.

The nucleic acid sequence of B3256 from Escherichia coli, e.g. as shown in the respective line in column 5 of Table I, application no. 13, has been published in B. And the activity of the gene product thereof is the activity of acetyl CoA carboxylase.

Accordingly, in one embodiment, the process of the present invention for producing violaxanthin in a non-human organism, like a microorganism or a plant or a part thereof, comprises increasing or generating the activity of a gene product with the activity of a gene product conferring the activity of “acetyl CoA carboxylase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical violaxanthin), application no. 13, preferably the coding region thereof, or a homolog or a fragment thereof, and being depicted in the same respective line as said B3256, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said B3256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said B3256, or a functional equivalent or a homolog thereof as depicted in column 8 of Table II, application no. 13, preferably a homolog or functional equivalent as depicted in column 8 of Table II B, application no. 13, and being depicted in the same respective line as said B3256, and preferably the activity is increased non-targeted, whereby the respective line disclose in column 7 the fine chemical violaxanthin.

Accordingly, in one embodiment, the molecule which activity is to be increased in the process of the invention is the gene product with an activity as a “acetyl CoA carboxylase”, preferably it is the molecule of section (a) or (b) of this paragraph.

In particular, it was observed that in plants, especially in Arabidopsis thaliana, increasing or generating the activity of a gene product non-targeted with the activity of a “acetyl CoA carboxylase”, preferably being encoded by a gene comprising the nucleic acid sequence SEQ ID NO. 9492, preferably the coding region thereof, conferred the production of or the increase in violaxanthin compared with the wild type control.

The nucleic acid sequence of B3346 from Escherichia coli, e.g. as shown in the respective line in column 5 of Table I, application no. 13, has been published in B. And the activity of the gene product thereof is the activity of b3346-protein.

Accordingly, in one embodiment, the process of the present invention for producing cryptoxanthin in a non-human organism, like a microorganism or a plant or a part thereof, comprises increasing or generating the activity of a gene product with the activity of a gene product conferring the activity of “b3346-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical cryptoxanthin), application no. 13, preferably the coding region thereof, or a homolog or a fragment thereof, and being depicted in the same respective line as said B3346, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said B3346, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said B3346, or a functional equivalent or a homolog thereof as depicted in column 8 of Table II, application no. 13, preferably a homolog or functional equivalent as depicted in column 8 of Table II B, application no. 13, and being depicted in the same respective line as said B3346, and preferably the activity is increased non-targeted, whereby the respective line disclose in column 7 the fine chemical cryptoxanthin.

Accordingly, in one embodiment, the molecule which activity is to be increased in the process of the invention is the gene product with an activity as a “b3346-protein”, preferably it is the molecule of section (a) or (b) of this paragraph.

In particular, it was observed that in plants, especially in Arabidopsis thaliana, increasing or generating the activity of a gene product non-targeted with the activity of a “b3346-protein”, preferably being encoded by a gene comprising the nucleic acid sequence SEQ ID NO. 10104, preferably the coding region thereof, conferred the production of or the increase in cryptoxanthin compared with the wild type control.

The nucleic acid sequence of B3774 from Escherichia coli, e.g. as shown in the respective line in column 5 of Table I, application no. 13, has been published in B. And the activity of the gene product thereof is the activity of ketol-acid reductoisomerase.

Accordingly, in one embodiment, the process of the present invention for producing violaxanthin in a non-human organism, like a microorganism or a plant or a part thereof, comprises increasing or generating the activity of a gene product with the activity of a gene product conferring the activity of “ketol-acid reductoisomerase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical violaxanthin), application no. 13, preferably the coding region thereof, or a homolog or a fragment thereof, and being depicted in the same respective line as said B3774, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said B3774, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said B3774, or a functional equivalent or a homolog thereof as depicted in column 8 of Table II, application no. 13, preferably a homolog or functional equivalent as depicted in column 8 of Table II B, application no. 13, and being depicted in the same respective line as said B3774, and preferably the activity is increased non-targeted, whereby the respective line disclose in column 7 the fine chemical violaxanthin.

Accordingly, in one embodiment, the molecule which activity is to be increased in the process of the invention is the gene product with an activity as a “ketol-acid reductoisomerase”, preferably it is the molecule of section (a) or (b) of this paragraph.

In particular, it was observed that in plants, especially in Arabidopsis thaliana, increasing or generating the activity of a gene product non-targeted with the activity of a “ketol-acid reductoisomerase”, preferably being encoded by a gene comprising the nucleic acid sequence SEQ ID NO. 94386, preferably the coding region thereof, conferred the production of or the increase in violaxanthin compared with the wild type control.

The nucleic acid sequence of B3817 from Escherichia coli, e.g. as shown in the respective line in column 5 of Table I, application no. 13, has been published in B. And the activity of the gene product thereof is the activity of b3817-protein.

Accordingly, in one embodiment, the process of the present invention for producing cryptoxanthin in a non-human organism, like a microorganism or a plant or a part thereof, comprises increasing or generating the activity of a gene product with the activity of a gene product conferring the activity of “b3817-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical cryptoxanthin), application no. 13, preferably the coding region thereof, or a homolog or a fragment thereof, and being depicted in the same respective line as said B3817, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said B3817, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said B3817, or a functional equivalent or a homolog thereof as depicted in column 8 of Table II, application no. 13, preferably a homolog or functional equivalent as depicted in column 8 of Table II B, application no. 13, and being depicted in the same respective line as said B3817, and preferably the activity is increased non-targeted, whereby the respective line disclose in column 7 the fine chemical cryptoxanthin.

Accordingly, in one embodiment, the molecule which activity is to be increased in the process of the invention is the gene product with an activity as a “b3817-protein”, preferably it is the molecule of section (a) or (b) of this paragraph.

In particular, it was observed that in plants, especially in Arabidopsis thaliana, increasing or generating the activity of a gene product non-targeted with the activity of a “b3817-protein”, preferably being encoded by a gene comprising the nucleic acid sequence SEQ ID NO. 10708, preferably the coding region thereof, conferred the production of or the increase in cryptoxanthin compared with the wild type control.

The nucleic acid sequence of B3908 from Escherichia coli, e.g. as shown in the respective line in column 5 of Table I, application no. 13, has been published in B. And the activity of the gene product thereof is the activity of superoxide dismutase.

Accordingly, in one embodiment, the process of the present invention for producing violaxanthin in a non-human organism, like a microorganism or a plant or a part thereof, comprises increasing or generating the activity of a gene product with the activity of a gene product conferring the activity of “superoxide dismutase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical violaxanthin), application no. 13, preferably the coding region thereof, or a homolog or a fragment thereof, and being depicted in the same respective line as said B3908, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said B3908, and preferably the activity is increased plastidic, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said B3908, or a functional equivalent or a homolog thereof as depicted in column 8 of Table II, application no. 13, preferably a homolog or functional equivalent as depicted in column 8 of Table II B, application no. 13, and being depicted in the same respective line as said B3908, and preferably the activity is increased plastidic, whereby the respective line disclose in column 7 the fine chemical violaxanthin.

Accordingly, in one embodiment, the molecule which activity is to be increased in the process of the invention is the gene product with an activity as a “superoxide dismutase”, preferably it is the molecule of section (a) or (b) of this paragraph.

In particular, it was observed that in plants, especially in Arabidopsis thaliana, increasing or generating the activity of a gene product plastidic with the activity of a “superoxide dismutase”, preferably being encoded by a gene comprising the nucleic acid sequence SEQ ID NO. 105659, preferably the coding region thereof, conferred the production of or the increase in violaxanthin compared with the wild type control.

The nucleic acid sequence of B4012 from Escherichia coli, e.g. as shown in the respective line in column 5 of Table I, application no. 13, has been published in B. And the activity of the gene product thereof is the activity of acetyltransferase.

Accordingly, in one embodiment, the process of the present invention for producing violaxanthin in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical violaxanthin), application no. 13, preferably the coding region thereof, or a homolog or a fragment thereof, and being depicted in the same respective line as said B4012, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said B4012, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said B4012, or a functional equivalent or a homolog thereof as depicted in column 8 of Table II, application no. 13, preferably a homolog or functional equivalent as depicted in column 8 of Table II B, application no. 13, and being depicted in the same respective line as said B4012, and preferably the activity is increased non-targeted, whereby the respective line disclose in column 7 the fine chemical violaxanthin.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 44378, preferably the coding region thereof, conferred the production of or the increase in violaxanthin compared with the wild type control.

The nucleic acid sequence of B4120 from Escherichia coli, e.g. as shown in the respective line in column 5 of Table I, application no. 13, has been published in B. And the activity of the gene product thereof is the activity of melibiose carrier protein.

Accordingly, in one embodiment, the process of the present invention for producing cryptoxanthin in a non-human organism, like a microorganism or a plant or a part thereof, comprises increasing or generating the activity of a gene product with the activity of a gene product conferring the activity of “melibiose carrier protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical cryptoxanthin), application no. 13, preferably the coding region thereof, or a homolog or a fragment thereof, and being depicted in the same respective line as said B4120, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said B4120, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said B4120, or a functional equivalent or a homolog thereof as depicted in column 8 of Table II, application no. 13, preferably a homolog or functional equivalent as depicted in column 8 of Table II B, application no. 13, and being depicted in the same respective line as said B4120, and preferably the activity is increased non-targeted, whereby the respective line disclose in column 7 the fine chemical cryptoxanthin.

Accordingly, in one embodiment, the molecule which activity is to be increased in the process of the invention is the gene product with an activity as a “melibiose carrier protein”, preferably it is the molecule of section (a) or (b) of this paragraph.

In particular, it was observed that in plants, especially in Arabidopsis thaliana, increasing or generating the activity of a gene product non-targeted with the activity of a “melibiose carrier protein”, preferably being encoded by a gene comprising the nucleic acid sequence SEQ ID NO. 130673, preferably the coding region thereof, conferred the production of or the increase in cryptoxanthin compared with the wild type control.

The nucleic acid sequence of B4120 from Escherichia coli, e.g. as shown in the respective line in column 5 of Table I, application no. 13, has been published in B. And the activity of the gene product thereof is the activity of melibiose carrier protein.

Accordingly, in one embodiment, the process of the present invention for producing zeaxanthin in a non-human organism, like a microorganism or a plant or a part thereof, comprises increasing or generating the activity of a gene product with the activity of a gene product conferring the activity of “melibiose carrier protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical zeaxanthin), application no. 13, preferably the coding region thereof, or a homolog or a fragment thereof, and being depicted in the same respective line as said B4120, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said B4120, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said B4120, or a functional equivalent or a homolog thereof as depicted in column 8 of Table II, application no. 13, preferably a homolog or functional equivalent as depicted in column 8 of Table II B, application no. 13, and being depicted in the same respective line as said B4120, and preferably the activity is increased non-targeted, whereby the respective line disclose in column 7 the fine chemical zeaxanthin.

Accordingly, in one embodiment, the molecule which activity is to be increased in the process of the invention is the gene product with an activity as a “melibiose carrier protein”, preferably it is the molecule of section (a) or (b) of this paragraph.

In particular, it was observed that in plants, especially in Arabidopsis thaliana, increasing or generating the activity of a gene product non-targeted with the activity of a “melibiose carrier protein”, preferably being encoded by a gene comprising the nucleic acid sequence SEQ ID NO. 130673, preferably the coding region thereof, conferred the production of or the increase in zeaxanthin compared with the wild type control.

The nucleic acid sequence of B4384 from Escherichia coli, e.g. as shown in the respective line in column 5 of Table I, application no. 13, has been published in B. And the activity of the gene product thereof is the activity of purine-nucleoside phosphorylase.

Accordingly, in one embodiment, the process of the present invention for producing violaxanthin in a non-human organism, like a microorganism or a plant or a part thereof, comprises increasing or generating the activity of a gene product with the activity of a gene product conferring the activity of “purine-nucleoside phosphorylase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical violaxanthin), application no. 13, preferably the coding region thereof, or a homolog or a fragment thereof, and being depicted in the same respective line as said B4384, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said B4384, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said B4384, or a functional equivalent or a homolog thereof as depicted in column 8 of Table II, application no. 13, preferably a homolog or functional equivalent as depicted in column 8 of Table II B, application no. 13, and being depicted in the same respective line as said B4384, and preferably the activity is increased non-targeted, whereby the respective line disclose in column 7 the fine chemical violaxanthin.

Accordingly, in one embodiment, the molecule which activity is to be increased in the process of the invention is the gene product with an activity as a “purine-nucleoside phosphorylase”, preferably it is the molecule of section (a) or (b) of this paragraph.

In particular, it was observed that in plants, especially in Arabidopsis thaliana, increasing or generating the activity of a gene product non-targeted with the activity of a “purine-nucleoside phosphorylase”, preferably being encoded by a gene comprising the nucleic acid sequence SEQ ID NO. 45556, preferably the coding region thereof, conferred the production of or the increase in violaxanthin compared with the wild type control.

The nucleic acid sequence of GM02LC19289 from Glycine max, e.g. as shown in the respective line in column 5 of Table I, application no. 13, is unpublished. And the activity of the gene product thereof is the activity of transcription factor.

Accordingly, in one embodiment, the process of the present invention for producing zeaxanthin in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical zeaxanthin), application no. 13, preferably the coding region thereof, or a homolog or a fragment thereof, and being depicted in the same respective line as said GM02LC19289, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said GM02LC19289, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said GM02LC19289, or a functional equivalent or a homolog thereof as depicted in column 8 of Table II, application no. 13, preferably a homolog or functional equivalent as depicted in column 8 of Table I I B, application no. 13, and being depicted in the same respective line as said GM02LC19289, and preferably the activity is increased non-targeted, whereby the respective line disclose in column 7 the fine chemical zeaxanthin.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 46751, preferably the coding region thereof, conferred the production of or the increase in zeaxanthin compared with the wild type control.

The nucleic acid sequence of Sll1185 from Synechocystis sp., e.g. as shown in the respective line in column 5 of Table I, application no. 13, has been published in C. And the activity of the gene product thereof is the activity of coproporphyrinogen oxidase.

Accordingly, in one embodiment, the process of the present invention for producing cryptoxanthin in a non-human organism, like a microorganism or a plant or a part thereof, comprises increasing or generating the activity of a gene product with the activity of a gene product conferring the activity of “coproporphyrinogen oxidase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical cryptoxanthin), application no. 13, preferably the coding region thereof, or a homolog or a fragment thereof, and being depicted in the same respective line as said Sll1185, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said Sll1185, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said Sll1185, or a functional equivalent or a homolog thereof as depicted in column 8 of Table II, application no. 13, preferably a homolog or functional equivalent as depicted in column 8 of Table II B, application no. 13, and being depicted in the same respective line as said Sll1185, and preferably the activity is increased non-targeted, whereby the respective line disclose in column 7 the fine chemical cryptoxanthin.

Accordingly, in one embodiment, the molecule which activity is to be increased in the process of the invention is the gene product with an activity as a “coproporphyrinogen oxidase”, preferably it is the molecule of section (a) or (b) of this paragraph.

In particular, it was observed that in plants, especially in Arabidopsis thaliana, increasing or generating the activity of a gene product non-targeted with the activity of a “coproporphyrinogen oxidase”, preferably being encoded by a gene comprising the nucleic acid sequence SEQ ID NO. 53189, preferably the coding region thereof, conferred the production of or the increase in cryptoxanthin compared with the wild type control.

The nucleic acid sequence of Sll1185 from Synechocystis sp., e.g. as shown in the respective line in column 5 of Table I, application no. 13, has been published in C. And the activity of the gene product thereof is the activity of coproporphyrinogen oxidase.

Accordingly, in one embodiment, the process of the present invention for producing zeaxanthin in a non-human organism, like a microorganism or a plant or a part thereof, comprises increasing or generating the activity of a gene product with the activity of a gene product conferring the activity of “coproporphyrinogen oxidase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical zeaxanthin), application no. 13, preferably the coding region thereof, or a homolog or a fragment thereof, and being depicted in the same respective line as said Sll1185, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said Sll1185, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said Sll1185, or a functional equivalent or a homolog thereof as depicted in column 8 of Table II, application no. 13, preferably a homolog or functional equivalent as depicted in column 8 of Table II B, application no. 13, and being depicted in the same respective line as said Sll1185, and preferably the activity is increased non-targeted, whereby the respective line disclose in column 7 the fine chemical zeaxanthin.

Accordingly, in one embodiment, the molecule which activity is to be increased in the process of the invention is the gene product with an activity as a “coproporphyrinogen oxidase”, preferably it is the molecule of section (a) or (b) of this paragraph.

In particular, it was observed that in plants, especially in Arabidopsis thaliana, increasing or generating the activity of a gene product non-targeted with the activity of a “coproporphyrinogen oxidase”, preferably being encoded by a gene comprising the nucleic acid sequence SEQ ID NO. 53189, preferably the coding region thereof, conferred the production of or the increase in zeaxanthin compared with the wild type control.

The nucleic acid sequence of Slr1755 from Synechocystis sp., e.g. as shown in the respective line in column 5 of Table I, application no. 13, has been published in C. And the activity of the gene product thereof is the activity of glycerol-3-phosphate dehydrogenase.

Accordingly, in one embodiment, the process of the present invention for producing zeaxanthin in a non-human organism, like a microorganism or a plant or a part thereof, comprises increasing or generating the activity of a gene product with the activity of a gene product conferring the activity of “glycerol-3-phosphate dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical zeaxanthin), application no. 13, preferably the coding region thereof, or a homolog or a fragment thereof, and being depicted in the same respective line as said Slr1755, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said Slr1755, and preferably the activity is increased plastidic, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said Slr1755, or a functional equivalent or a homolog thereof as depicted in column 8 of Table II, application no. 13, preferably a homolog or functional equivalent as depicted in column 8 of Table II B, application no. 13, and being depicted in the same respective line as said Slr1755, and preferably the activity is increased plastidic, whereby the respective line disclose in column 7 the fine chemical zeaxanthin.

Accordingly, in one embodiment, the molecule which activity is to be increased in the process of the invention is the gene product with an activity as a “glycerol-3-phosphate dehydrogenase”, preferably it is the molecule of section (a) or (b) of this paragraph.

In particular, it was observed that in plants, especially in Arabidopsis thaliana, increasing or generating the activity of a gene product plastidic with the activity of a “glycerol-3-phosphate dehydrogenase”, preferably being encoded by a gene comprising the nucleic acid sequence SEQ ID NO. 58823, preferably the coding region thereof, conferred the production of or the increase in zeaxanthin compared with the wild type control.

The nucleic acid sequence of YCl026c-a from Saccharomyces cerevisiae, e.g. as shown in the respective line in column 5 of Table I, application no. 13, has been published in A. And the activity of the gene product thereof is the activity of YCL026C-A-protein.

Accordingly, in one embodiment, the process of the present invention for producing cryptoxanthin in a non-human organism, like a microorganism or a plant or a part thereof, comprises increasing or generating the activity of a gene product with the activity of a gene product conferring the activity of “YCL026C-A-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical cryptoxanthin), application no. 13, preferably the coding region thereof, or a homolog or a fragment thereof, and being depicted in the same respective line as said YCl026c-a, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said YCl026c-a, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said YCl026c-a, or a functional equivalent or a homolog thereof as depicted in column 8 of Table II, application no. 13, preferably a homolog or functional equivalent as depicted in column 8 of Table II B, application no. 13, and being depicted in the same respective line as said YCl026c-a, and preferably the activity is increased non-targeted, whereby the respective line disclose in column 7 the fine chemical cryptoxanthin.

Accordingly, in one embodiment, the molecule which activity is to be increased in the process of the invention is the gene product with an activity as a “YCL026C-A-protein”, preferably it is the molecule of section (a) or (b) of this paragraph.

In particular, it was observed that in plants, especially in Arabidopsis thaliana, increasing or generating the activity of a gene product non-targeted with the activity of a “YCL026C-A-protein”, preferably being encoded by a gene comprising the nucleic acid sequence SEQ ID NO. 63167, preferably the coding region thereof, conferred the production of or the increase in cryptoxanthin compared with the wild type control.

The nucleic acid sequence of Ydr131c from Saccharomyces cerevisiae, e.g. as shown in the respective line in column 5 of Table I, application no. 13, has been published in A. And the activity of the gene product thereof is the activity of F-box protein.

Accordingly, in one embodiment, the process of the present invention for producing cryptoxanthin in a non-human organism, like a microorganism or a plant or a part thereof, comprises increasing or generating the activity of a gene product with the activity of a gene product conferring the activity of “F-box protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical cryptoxanthin), application no. 13, preferably the coding region thereof, or a homolog or a fragment thereof, and being depicted in the same respective line as said Ydr131c, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said Ydr131c, and preferably the activity is increased plastidic, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said Ydr131c, or a functional equivalent or a homolog thereof as depicted in column 8 of Table II, application no. 13, preferably a homolog or functional equivalent as depicted in column 8 of Table II B, application no. 13, and being depicted in the same respective line as said Ydr131c, and preferably the activity is increased plastidic, whereby the respective line disclose in column 7 the fine chemical cryptoxanthin.

Accordingly, in one embodiment, the molecule which activity is to be increased in the process of the invention is the gene product with an activity as a “F-box protein”, preferably it is the molecule of section (a) or (b) of this paragraph.

In particular, it was observed that in plants, especially in Arabidopsis thaliana, increasing or generating the activity of a gene product plastidic with the activity of a “F-box protein”, preferably being encoded by a gene comprising the nucleic acid sequence SEQ ID NO. 14706, preferably the coding region thereof, conferred the production of or the increase in cryptoxanthin compared with the wild type control.

The nucleic acid sequence of Yg1096w from Saccharomyces cerevisiae, e.g. as shown in the respective line in column 5 of Table I, application no. 13, has been published in A. And the activity of the gene product thereof is the activity of ygl096w-protein.

Accordingly, in one embodiment, the process of the present invention for producing cryptoxanthin in a non-human organism, like a microorganism or a plant or a part thereof, comprises increasing or generating the activity of a gene product with the activity of a gene product conferring the activity of “ygl096w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical cryptoxanthin), application no. 13, preferably the coding region thereof, or a homolog or a fragment thereof, and being depicted in the same respective line as said Ygl096w, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said Yg1096w, and preferably the activity is increased plastidic, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said Ygl096w, or a functional equivalent or a homolog thereof as depicted in column 8 of Table II, application no. 13, preferably a homolog or functional equivalent as depicted in column 8 of Table II B, application no. 13, and being depicted in the same respective line as said Ygl096w, and preferably the activity is increased plastidic, whereby the respective line disclose in column 7 the fine chemical cryptoxanthin.

Accordingly, in one embodiment, the molecule which activity is to be increased in the process of the invention is the gene product with an activity as a “ygl096w-protein”, preferably it is the molecule of section (a) or (b) of this paragraph.

In particular, it was observed that in plants, especially in Arabidopsis thaliana, increasing or generating the activity of a gene product plastidic with the activity of a “ygl096w-protein”, preferably being encoded by a gene comprising the nucleic acid sequence SEQ ID NO. 90278, preferably the coding region thereof, conferred the production of or the increase in cryptoxanthin compared with the wild type control.

The nucleic acid sequence of Ygl256w from Saccharomyces cerevisiae, e.g. as shown in the respective line in column 5 of Table I, application no. 13, has been published in A. And the activity of the gene product thereof is the activity of alcohol dehydrogenase.

Accordingly, in one embodiment, the process of the present invention for producing cryptoxanthin in a non-human organism, like a microorganism or a plant or a part thereof, comprises increasing or generating the activity of a gene product with the activity of a gene product conferring the activity of “alcohol dehydrogenase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical cryptoxanthin), application no. 13, preferably the coding region thereof, or a homolog or a fragment thereof, and being depicted in the same respective line as said Ygl256w, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said Ygl256w, and preferably the activity is increased plastidic, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said Ygl256w, or a functional equivalent or a homolog thereof as depicted in column 8 of Table II, application no. 13, preferably a homolog or functional equivalent as depicted in column 8 of Table II B, application no. 13, and being depicted in the same respective line as said Ygl256w, and preferably the activity is increased plastidic, whereby the respective line disclose in column 7 the fine chemical cryptoxanthin.

Accordingly, in one embodiment, the molecule which activity is to be increased in the process of the invention is the gene product with an activity as a “alcohol dehydrogenase”, preferably it is the molecule of section (a) or (b) of this paragraph.

In particular, it was observed that in plants, especially in Arabidopsis thaliana, increasing or generating the activity of a gene product plastidic with the activity of a “alcohol dehydrogenase”, preferably being encoded by a gene comprising the nucleic acid sequence SEQ ID NO. 130742, preferably the coding region thereof, conferred the production of or the increase in cryptoxanthin compared with the wild type control.

The nucleic acid sequence of Ykr072c from Saccharomyces cerevisiae, e.g. as shown in the respective line in column 5 of Table I, application no. 13, has been published in A. And the activity of the gene product thereof is the activity of halotolerance protein.

Accordingly, in one embodiment, the process of the present invention for producing cryptoxanthin in a non-human organism, like a microorganism or a plant or a part thereof, comprises increasing or generating the activity of a gene product with the activity of a gene product conferring the activity of “halotolerance protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical cryptoxanthin), application no. 13, preferably the coding region thereof, or a homolog or a fragment thereof, and being depicted in the same respective line as said Ykr072c, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said Ykr072c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said Ykr072c, or a functional equivalent or a homolog thereof as depicted in column 8 of Table II, application no. 13, preferably a homolog or functional equivalent as depicted in column 8 of Table II B, application no. 13, and being depicted in the same respective line as said Ykr072c, and preferably the activity is increased non-targeted, whereby the respective line disclose in column 7 the fine chemical cryptoxanthin.

Accordingly, in one embodiment, the molecule which activity is to be increased in the process of the invention is the gene product with an activity as a “halotolerance protein”, preferably it is the molecule of section (a) or (b) of this paragraph.

In particular, it was observed that in plants, especially in Arabidopsis thaliana, increasing or generating the activity of a gene product non-targeted with the activity of a “halotolerance protein”, preferably being encoded by a gene comprising the nucleic acid sequence SEQ ID NO. 130903, preferably the coding region thereof, conferred the production of or the increase in cryptoxanthin compared with the wild type control.

The nucleic acid sequence of Ykr072c from Saccharomyces cerevisiae, e.g. as shown in the respective line in column 5 of Table I, application no. 13, has been published in A. And the activity of the gene product thereof is the activity of halotolerance protein.

Accordingly, in one embodiment, the process of the present invention for producing zeaxanthin in a non-human organism, like a microorganism or a plant or a part thereof, comprises increasing or generating the activity of a gene product with the activity of a gene product conferring the activity of “halotolerance protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical zeaxanthin), application no. 13, preferably the coding region thereof, or a homolog or a fragment thereof, and being depicted in the same respective line as said Ykr072c, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said Ykr072c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said Ykr072c, or a functional equivalent or a homolog thereof as depicted in column 8 of Table II, application no. 13, preferably a homolog or functional equivalent as depicted in column 8 of Table II B, application no. 13, and being depicted in the same respective line as said Ykr072c, and preferably the activity is increased non-targeted, whereby the respective line disclose in column 7 the fine chemical zeaxanthin.

Accordingly, in one embodiment, the molecule which activity is to be increased in the process of the invention is the gene product with an activity as a “halotolerance protein”, preferably it is the molecule of section (a) or (b) of this paragraph.

In particular, it was observed that in plants, especially in Arabidopsis thaliana, increasing or generating the activity of a gene product non-targeted with the activity of a “halotolerance protein”, preferably being encoded by a gene comprising the nucleic acid sequence SEQ ID NO. 130903, preferably the coding region thereof, conferred the production of or the increase in zeaxanthin compared with the wild type control.

The nucleic acid sequence of Ymr281w from Saccharomyces cerevisiae, e.g. as shown in the respective line in column 5 of Table I, application no. 13, has been published in A. And the activity of the gene product thereof is the activity of N-acetylglucosaminylphosphatidylinositoldeacetylase.

Accordingly, in one embodiment, the process of the present invention for producing zeaxanthin in a non-human organism, like a microorganism or a plant or a part thereof, comprises increasing or generating the activity of a gene product with the activity of a gene product conferring the activity of “N-acetylglucosaminylphosphatidylinositoldeacetylase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical zeaxanthin), application no. 13, preferably the coding region thereof, or a homolog or a fragment thereof, and being depicted in the same respective line as said Ymr281w, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said Ymr281w, and preferably the activity is increased plastidic, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said Ymr281w, or a functional equivalent or a homolog thereof as depicted in column 8 of Table II, application no. 13, preferably a homolog or functional equivalent as depicted in column 8 of Table II B, application no. 13, and being depicted in the same respective line as said Ymr281w, and preferably the activity is increased plastidic, whereby the respective line disclose in column 7 the fine chemical zeaxanthin.

Accordingly, in one embodiment, the molecule which activity is to be increased in the process of the invention is the gene product with an activity as a “N-acetylglucosaminylphosphatidylinositoldeacetylase”, preferably it is the molecule of section (a) or (b) of this paragraph.

In particular, it was observed that in plants, especially in Arabidopsis thaliana, increasing or generating the activity of a gene product plastidic with the activity of a “N-acetylglucosaminylphosphatidylinositoldeacetylase”, preferably being encoded by a gene comprising the nucleic acid sequence SEQ ID NO. 130932, preferably the coding region thereof, conferred the production of or the increase in zeaxanthin compared with the wild type control.

The nucleic acid sequence of Ymr324c from Saccharomyces cerevisiae, e.g. as shown in the respective line in column 5 of Table I, application no. 13, has been published in A. And the activity of the gene product thereof is the activity of ymr324c-protein.

Accordingly, in one embodiment, the process of the present invention for producing cryptoxanthin in a non-human organism, like a microorganism or a plant or a part thereof, comprises increasing or generating the activity of a gene product with the activity of a gene product conferring the activity of “ymr324c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical cryptoxanthin), application no. 13, preferably the coding region thereof, or a homolog or a fragment thereof, and being depicted in the same respective line as said Ymr324c, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said Ymr324c, and preferably the activity is increased plastidic, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said Ymr324c, or a functional equivalent or a homolog thereof as depicted in column 8 of Table II, application no. 13, preferably a homolog or functional equivalent as depicted in column 8 of Table II B, application no. 13, and being depicted in the same respective line as said Ymr324c, and preferably the activity is increased plastidic, whereby the respective line disclose in column 7 the fine chemical cryptoxanthin.

Accordingly, in one embodiment, the molecule which activity is to be increased in the process of the invention is the gene product with an activity as a “ymr324c-protein”, preferably it is the molecule of section (a) or (b) of this paragraph.

In particular, it was observed that in plants, especially in Arabidopsis thaliana, increasing or generating the activity of a gene product plastidic with the activity of a “ymr324c-protein”, preferably being encoded by a gene comprising the nucleic acid sequence SEQ ID NO. 130986, preferably the coding region thereof, conferred the production of or the increase in cryptoxanthin compared with the wild type control.

The nucleic acid sequence of Ynr019w from Saccharomyces cerevisiae, e.g. as shown in the respective line in column 5 of Table I, application no. 13, has been published in A. And the activity of the gene product thereof is the activity of sterol O-acyltransferase.

Accordingly, in one embodiment, the process of the present invention for producing zeaxanthin in a non-human organism, like a microorganism or a plant or a part thereof, comprises increasing or generating the activity of a gene product with the activity of a gene product conferring the activity of “sterol O-acyltransferase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical zeaxanthin), application no. 13, preferably the coding region thereof, or a homolog or a fragment thereof, and being depicted in the same respective line as said Ynr019w, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said Ynr019w, and preferably the activity is increased plastidic, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said Ynr019w, or a functional equivalent or a homolog thereof as depicted in column 8 of Table II, application no. 13, preferably a homolog or functional equivalent as depicted in column 8 of Table II B, application no. 13, and being depicted in the same respective line as said Ynr019w, and preferably the activity is increased plastidic, whereby the respective line disclose in column 7 the fine chemical zeaxanthin.

Accordingly, in one embodiment, the molecule which activity is to be increased in the process of the invention is the gene product with an activity as a “sterol O-acyltransferase”, preferably it is the molecule of section (a) or (b) of this paragraph.

In particular, it was observed that in plants, especially in Arabidopsis thaliana, increasing or generating the activity of a gene product plastidic with the activity of a “sterol O-acyltransferase”, preferably being encoded by a gene comprising the nucleic acid sequence SEQ ID NO. 67646, preferably the coding region thereof, conferred the production of or the increase in zeaxanthin compared with the wild type control.

The nucleic acid sequence of Yer184c from Saccharomyces cerevisiae, e.g. as shown in the respective line in column 5 of Table I, application no. 13, has been published in A. And the activity of the gene product thereof is the activity of YER184C-protein.

Accordingly, in one embodiment, the process of the present invention for producing abscisic acid in a non-human organism, like a microorganism or a plant or a part thereof, comprises increasing or generating the activity of a gene product with the activity of a gene product conferring the activity of “YER184C-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical abscisic acid), application no. 13, preferably the coding region thereof, or a homolog or a fragment thereof, and being depicted in the same respective line as said Yer184c, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said Yer184c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said Yer184c, or a functional equivalent or a homolog thereof as depicted in column 8 of Table II, application no. 13, preferably a homolog or functional equivalent as depicted in column 8 of Table II B, application no. 13, and being depicted in the same respective line as said Yer184c, and preferably the activity is increased non-targeted, whereby the respective line disclose in column 7 the fine chemical abscisic acid.

Accordingly, in one embodiment, the molecule which activity is to be increased in the process of the invention is the gene product with an activity as a “YER184C-protein”, preferably it is the molecule of section (a) or (b) of this paragraph.

In particular, it was observed that in plants, especially in Arabidopsis thaliana, increasing or generating the activity of a gene product non-targeted with the activity of a “YER184C-protein”, preferably being encoded by a gene comprising the nucleic acid sequence SEQ ID NO. 130713, preferably the coding region thereof, conferred the production of or the increase in abscisic acid compared with the wild type control.

The nucleic acid sequence of YOR044W from Saccharomyces cerevisiae, e.g. as shown in the respective line in column 5 of Table I, application no. 13, has been published in A. And the activity of the gene product thereof is the activity of YOR044W-protein.

Accordingly, in one embodiment, the process of the present invention for producing abscisic acid in a non-human organism, like a microorganism or a plant or a part thereof, comprises increasing or generating the activity of a gene product with the activity of a gene product conferring the activity of “YOR044W-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical abscisic acid), application no. 13, preferably the coding region thereof, or a homolog or a fragment thereof, and being depicted in the same respective line as said YOR044W, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said YOR044W, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said YOR044W, or a functional equivalent or a homolog thereof as depicted in column 8 of Table II, application no. 13, preferably a homolog or functional equivalent as depicted in column 8 of Table II B, application no. 13, and being depicted in the same respective line as said YOR044W, and preferably the activity is increased non-targeted, whereby the respective line disclose in column 7 the fine chemical abscisic acid.

Accordingly, in one embodiment, the molecule which activity is to be increased in the process of the invention is the gene product with an activity as a “YOR044W-protein”, preferably it is the molecule of section (a) or (b) of this paragraph.

In particular, it was observed that in plants, especially in Arabidopsis thaliana, increasing or generating the activity of a gene product non-targeted with the activity of a “YOR044W-protein”, preferably being encoded by a gene comprising the nucleic acid sequence SEQ ID NO. 130999, preferably the coding region thereof, conferred the production of or the increase in abscisic acid compared with the wild type control.

The nucleic acid sequence of B4321 from Escherichia coli, e.g. as shown in the respective line in column 5 of Table I, application no. 13, has been published in B. And the activity of the gene product thereof is the activity of gluconate transport system permease 3.

Accordingly, in one embodiment, the process of the present invention for producing abscisic acid in a non-human organism, like a microorganism or a plant or a part thereof, comprises increasing or generating the activity of a gene product with the activity of a gene product conferring the activity of “gluconate transport system permease 3”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical abscisic acid), application no. 13, preferably the coding region thereof, or a homolog or a fragment thereof, and being depicted in the same respective line as said B4321, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said B4321, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said B4321, or a functional equivalent or a homolog thereof as depicted in column 8 of Table II, application no. 13, preferably a homolog or functional equivalent as depicted in column 8 of Table II B, application no. 13, and being depicted in the same respective line as said B4321, and preferably the activity is increased non-targeted, whereby the respective line disclose in column 7 the fine chemical abscisic acid.

Accordingly, in one embodiment, the molecule which activity is to be increased in the process of the invention is the gene product with an activity as a “gluconate transport system permease 3”, preferably it is the molecule of section (a) or (b) of this paragraph.

In particular, it was observed that in plants, especially in Arabidopsis thaliana, increasing or generating the activity of a gene product non-targeted with the activity of a “gluconate transport system permease 3”, preferably being encoded by a gene comprising the nucleic acid sequence SEQ ID NO. 45394, preferably the coding region thereof, conferred the production of or the increase in abscisic acid compared with the wild type control.

The nucleic acid sequence of Sll1450 from Synechocystis sp., e.g. as shown in the respective line in column 5 of Table I, application no. 13, has been published in C. And the activity of the gene product thereof is the activity of nitrate/nitrite transport protein.

Accordingly, in one embodiment, the process of the present invention for producing abscisic acid in a non-human organism, like a microorganism or a plant or a part thereof, comprises increasing or generating the activity of a gene product with the activity of a gene product conferring the activity of “nitrate/nitrite transport protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical abscisic acid), application no. 13, preferably the coding region thereof, or a homolog or a fragment thereof, and being depicted in the same respective line as said Sll1450, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said Sll1450, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said Sll1450, or a functional equivalent or a homolog thereof as depicted in column 8 of Table II, application no. 13, preferably a homolog or functional equivalent as depicted in column 8 of Table II B, application no. 13, and being depicted in the same respective line as said Sll1450, and preferably the activity is increased mitochondrial, whereby the respective line disclose in column 7 the fine chemical abscisic acid.

Accordingly, in one embodiment, the molecule which activity is to be increased in the process of the invention is the gene product with an activity as a “nitrate/nitrite transport protein”, preferably it is the molecule of section (a) or (b) of this paragraph.

In particular, it was observed that in plants, especially in Arabidopsis thaliana, increasing or generating the activity of a gene product mitochondrial with the activity of a “nitrate/nitrite transport protein”, preferably being encoded by a gene comprising the nucleic acid sequence SEQ ID NO. 54337, preferably the coding region thereof, conferred the production of or the increase in abscisic acid compared with the wild type control.

The nucleic acid sequence of Sll1848 from Synechocystis sp., e.g. as shown in the respective line in column 5 of Table I, application no. 13, has been published in C. And the activity of the gene product thereof is the activity of acyl transferase.

Accordingly, in one embodiment, the process of the present invention for producing abscisic acid in a non-human organism, like a microorganism or a plant or a part thereof, comprises increasing or generating the activity of a gene product with the activity of a gene product conferring the activity of “acyl transferase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical abscisic acid), application no. 13, preferably the coding region thereof, or a homolog or a fragment thereof, and being depicted in the same respective line as said Sll1848, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said Sll1848, and preferably the activity is increased plastidic, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said Sll1848, or a functional equivalent or a homolog thereof as depicted in column 8 of Table II, application no. 13, preferably a homolog or functional equivalent as depicted in column 8 of Table II B, application no. 13, and being depicted in the same respective line as said Sll1848, and preferably the activity is increased plastidic, whereby the respective line disclose in column 7 the fine chemical abscisic acid.

Accordingly, in one embodiment, the molecule which activity is to be increased in the process of the invention is the gene product with an activity as a “acyl transferase”, preferably it is the molecule of section (a) or (b) of this paragraph.

In particular, it was observed that in plants, especially in Arabidopsis thaliana, increasing or generating the activity of a gene product plastidic with the activity of a “acyl transferase”, preferably being encoded by a gene comprising the nucleic acid sequence SEQ ID NO. 86447, preferably the coding region thereof, conferred the production of or the increase in abscisic acid compared with the wild type control.

The nucleic acid sequence of At3g02990 from Arabidopsis thaliana, e.g. as shown in the respective line in column 5 of Table I, application no. 13, has been published in F. And the activity of the gene product thereof is the activity of heat shock transcription factor.

Accordingly, in one embodiment, the process of the present invention for producing abscisic acid in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical abscisic acid), application no. 13, preferably the coding region thereof, or a homolog or a fragment thereof, and being depicted in the same respective line as said At3g02990, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said At3g02990, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said At3g02990, or a functional equivalent or a homolog thereof as depicted in column 8 of Table II, application no. 13, preferably a homolog or functional equivalent as depicted in column 8 of Table II B, application no. 13, and being depicted in the same respective line as said At3g02990, and preferably the activity is increased non-targeted, whereby the respective line disclose in column 7 the fine chemical abscisic acid.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 68657, preferably the coding region thereof, conferred the production of or the increase in abscisic acid compared with the wild type control.

The nucleic acid sequence of At1g48260 from Arabidopsis thaliana, e.g. as shown in the respective line in column 5 of Table I, application no. 13, has been published in F. And the activity of the gene product thereof is the activity of protein kinase.

Accordingly, in one embodiment, the process of the present invention for producing abscisic acid in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical abscisic acid), application no. 13, preferably the coding region thereof, or a homolog or a fragment thereof, and being depicted in the same respective line as said At1 g48260, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said At1g48260, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said At1g48260, or a functional equivalent or a homolog thereof as depicted in column 8 of Table II, application no. 13, preferably a homolog or functional equivalent as depicted in column 8 of Table II B, application no. 13, and being depicted in the same respective line as said At1 g48260, and preferably the activity is increased non-targeted, whereby the respective line disclose in column 7 the fine chemical abscisic acid.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 18235, preferably the coding region thereof, conferred the production of or the increase in abscisic acid compared with the wild type control.

The nucleic acid sequence of At1g72770 from Arabidopsis thaliana, e.g. as shown in the respective line in column 5 of Table I, application no. 13, has been published in F. And the activity of the gene product thereof is the activity of protein phosphatase.

Accordingly, in one embodiment, the process of the present invention for producing abscisic acid in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical abscisic acid), application no. 13, preferably the coding region thereof, or a homolog or a fragment thereof, and being depicted in the same respective line as said At1g72770, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said At1g72770, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said At1g72770, or a functional equivalent or a homolog thereof as depicted in column 8 of Table II, application no. 13, preferably a homolog or functional equivalent as depicted in column 8 of Table II B, application no. 13, and being depicted in the same respective line as said At1g72770, and preferably the activity is increased non-targeted, whereby the respective line disclose in column 7 the fine chemical abscisic acid.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19419, preferably the coding region thereof, conferred the production of or the increase in abscisic acid compared with the wild type control.

The nucleic acid sequence of AVINDRAFT_{—}0539 from Azotobacter vinelandii, e.g. as shown in the respective line in column 5 of Table I, application no. 13, has been published in D. And the activity of the gene product thereof is the activity of glycine cleavage system T aminomethyltransferase.

Accordingly, in one embodiment, the process of the present invention for producing abscisic acid in a non-human organism, like a microorganism or a plant or a part thereof, comprises increasing or generating the activity of a gene product with the activity of a gene product conferring the activity of “glycine cleavage system T aminomethyltransferase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical abscisic acid), application no. 13, preferably the coding region thereof, or a homolog or a fragment thereof, and being depicted in the same respective line as said AVINDRAFT_{—}0539, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said AVINDRAFT_{—}0539, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said AVINDRAFT_{—}0539, or a functional equivalent or a homolog thereof as depicted in column 8 of Table II, application no. 13, preferably a homolog or functional equivalent as depicted in column 8 of Table II B, application no. 13, and being depicted in the same respective line as said AVINDRAFT_{—}0539, and preferably the activity is increased non-targeted, whereby the respective line disclose in column 7 the fine chemical abscisic acid.

Accordingly, in one embodiment, the molecule which activity is to be increased in the process of the invention is the gene product with an activity as a “glycine cleavage system T aminomethyltransferase”, preferably it is the molecule of section (a) or (b) of this paragraph.

In particular, it was observed that in plants, especially in Arabidopsis thaliana, increasing or generating the activity of a gene product non-targeted with the activity of a “glycine cleavage system T aminomethyltransferase”, preferably being encoded by a gene comprising the nucleic acid sequence SEQ ID NO. 70715, preferably the coding region thereof, conferred the production of or the increase in abscisic acid compared with the wild type control.

The nucleic acid sequence of At2g30540 from Arabidopsis thaliana, e.g. as shown in the respective line in column 5 of Table I, application no. 13, has been published in F. And the activity of the gene product thereof is the activity of glutaredoxin.

Accordingly, in one embodiment, the process of the present invention for producing abscisic acid in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical abscisic acid), application no. 13, preferably the coding region thereof, or a homolog or a fragment thereof, and being depicted in the same respective line as said At2g30540, or a functional equivalent or a homolog thereof as shown in column 8 of Table I, application no. 13, preferably the coding region thereof, particularly a homolog or functional equivalent as shown in column 8 of Table I B, application no. 13, and being depicted in the same respective line as said At2g30540, and preferably the activity is increased non-targeted, or
(b) a polypeptide comprising a polypeptide, a consensus sequence or at least a polypeptide motif as shown in the respective line in column 5 of Table II or in column 8 of Table IV, application no. 13, respectively, and being depicted in the same respective line as said At2g30540, or a functional equivalent or a homolog thereof as depicted in column 8 of Table II, application no. 13, preferably a homolog or functional equivalent as depicted in column 8 of Table II B, application no. 13, and being depicted in the same respective line as said At2g30540, and preferably the activity is increased non-targeted, whereby the respective line disclose in column 7 the fine chemical abscisic acid.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 20346, preferably the coding region thereof, conferred the production of or the increase in abscisic acid compared with the wild type control.

[0105.1.7.13] to [0107.1.7.13] for the disclosure of these paragraphs see [0105.1.7.7] to [0107.1.7.7] above

A protein having an activity conferring an increase in the amount or level of the fine chemical abscisic acid, cryptoxanthin, violaxanthin, or zeaxanthin, upon targeting to the plastids or mitochondria or upon non-targeting, preferably has the structure of the respective polypeptide described herein, in particular of the polypeptides comprising the consensus sequence or at least one polypeptide motifs as shown in the respective line in Table IV, application no. 13, column 8 or of the polypeptide comprising an amino acid sequence as disclosed in the respective line in Table II, application no. 13, columns 5 or 8, or homologs or fragments thereof as described herein, or is encoded by the nucleic acid molecule characterized herein or the nucleic acid molecule according to the invention, for example by the nucleic acid molecule as shown in the respective line in Table I, application no. 13, columns 5 or 8, preferably the coding region thereof, or homologs or fragments thereof and has the herein mentioned activity.

for the disclosure of this paragraph see [0109.1.7.7] above

for the disclosure of this paragraph see [0110.1.7.7] above

In one embodiment, the process of the present invention comprises one or more of the following steps
(a) stabilizing a protein conferring the generation or increased expression of a protein encoded by the nucleic acid molecule of the invention or of the polypeptide of the invention, e.g. of a polypeptide having the activity selected from the group consisting of acetyl CoA carboxylase, acetyltransferase, acyl transferase, alcohol dehydrogenase, AX653549-protein, b1003-protein, b2032-protein, b2099-protein, b2121-protein, b2474-protein, b3346-protein, b3817-protein, coproporphyrinogen oxidase, F-box protein, flavodoxin, gluconate transport system permease 3, glutaredoxin, glycerol-3-phosphate dehydrogenase, glycine cleavage system T aminomethyltransferase, halotolerance protein, heat shock transcription factor, isopropylmalate isomerase large subunit, ketol-acid reductoisomerase, lipoprotein precursor, MADS-box transcription factor, melibiose carrier protein, monothiol glutaredoxin, monthiol glutaredoxin, N-acetylglucosaminylphosphatidylinositoldeacetylase, nitrate/nitrite transport protein, protein kinase, protein phosphatase, purine-nucleoside phosphorylase, ribosephosphate isomerase, constitutive, serine/threonine-protein phosphatase, sterol O-acyltransferase, superoxide dismutase, thioredoxin, transcription factor, transcriptional regulator, YCL026C-A-protein, YER184C-protein, ygl096w-protein, ymr324c-protein, and YOR044W-protein, or of a polypeptide as indicated in the respective line in Table II, application no. 13, columns 5 or 8, or its homologs or fragments, and conferring the production of or an increase in abscisic acid, cryptoxanthin, violaxanthin, or zeaxanthin, respectively; and/or
(b) stabilizing a mRNA conferring the generation or increased expression of a FCRP, e.g. protein encoded by the nucleic acid molecule of the invention or its homologs or fragments, or of a mRNA encoding the polypeptide of the present invention having the hereinmentioned activity selected from the group consisting of said activities mentioned in (a) and conferring the production of or an increase in abscisic acid, cryptoxanthin, violaxanthin, or zeaxanthin, respectively; and/or
(c) increasing the specific activity of a protein conferring the increased expression of a FCRP, e.g. a protein encoded by the nucleic acid molecule of the invention or of the polypeptide of the present invention having herein-mentioned abscisic acid, cryptoxanthin, violaxanthin, or zeaxanthin generating or increasing activity, respectively, e.g. of a polypeptide having the activity of a protein as indicated in the respective line in Table II, application no. 13, columns 5 or 8, or its homologs or fragments, or decreasing the inhibitory regulation of the polypeptide of the invention; and/or
(d) generating or increasing the expression of an endogenous or artificial transcription factor mediating the expression of a protein conferring the generation or increased expression of a FCRP, e.g. a protein encoded by the nucleic acid molecule of the invention or of the polypeptide of the invention having herein-mentioned activity selected from the group consisting of said activities mentioned in (a) and conferring a abscisic acid, cryptoxanthin, violaxanthin, or zeaxanthin increasing activity, respectively, e.g. of a polypeptide having the activity of a protein as indicated in the respective line in Table II, application no. 13, columns 5 or 8 or its homologs; and/or
(e) stimulating activity of a protein conferring the increased expression of a FCRP, e.g. a protein encoded by the nucleic acid molecule of the present invention or a polypeptide of the present invention having herein-mentioned activity selected from the group consisting of said activities mentioned in (a) and conferring a abscisic acid, cryptoxanthin, violaxanthin, or zeaxanthin increasing activity, respectively, e.g. of a polypeptide having the activity of a protein as indicated in the respective line in Table II, application no. 13, columns 5 or 8, or its homologs or fragments, by adding one or more exogenous inducing factors to the non-human organism or parts thereof; and/or
(f) expressing a transgenic gene encoding a protein conferring the increased expression of a FCRP, e.g. a polypeptide encoded by the nucleic acid molecule of the present invention or a polypeptide of the present invention, having herein-mentioned activity selected from the group consisting of said activities mentioned in (a) and conferring a abscisic acid, cryptoxanthin, violaxanthin, or zeaxanthin increasing activity, respectively, e.g. of a polypeptide having the activity of a protein as indicated in the respective line in Table II, application no. 13, columns 5 or 8, or its homologs or fragments, and/or
(g) increasing the copy number of a gene conferring the increased expression of a nucleic acid molecule encoding a FCRP, e.g. a polypeptide encoded by the nucleic acid molecule of the invention or the polypeptide of the invention having herein-mentioned activity selected from the group consisting of said activities mentioned in (a) and conferring a abscisic acid, cryptoxanthin, violaxanthin, or zeaxanthin increasing activity, respectively, e.g. of a polypeptide having the activity of a protein as indicated in the respective line in Table II, application no. 13, columns 5 or 8, or its homologs or fragments; and/or
(h) increasing the expression of the endogenous gene encoding the FCRP, polypeptide of the invention, e.g. a polypeptide encoded by the nucleic acid molecule of the invention or the polypeptide of the invention having herein-mentioned activity selected from the group consisting of said activities mentioned in (a) and conferring a abscisic acid, cryptoxanthin, violaxanthin, or zeaxanthin; increasing activity, respectively, e.g. of a polypeptide having the activity of a protein as indicated in the respective line in Table II, application no. 13, columns 5 or 8, or its homologs or fragments, by adding positive expression or removing negative expression elements, e.g. homologous recombination can be used to either introduce positive regulatory elements like for plants the 35S enhancer into the promoter or to remove repressor elements form regulatory regions. Further gene conversion methods can be used to disrupt repressor elements or to enhance to activity of positive elements. Positive elements can be randomly introduced in plants by T-DNA or transposon mutagenesis and lines can be identified in which the positive elements have be integrated near to a gene of the invention, the expression of which is thereby enhanced; and/or
(i) modulating growth conditions of the non-human organism in such a manner, that the expression or activity of the gene encoding the FCRP, e.g. a protein of the invention or the protein itself is enhanced, for example microorganisms or plants can be grown for example under a higher temperature regime leading to an enhanced expression of heat shock proteins, which can lead to an enhanced abscisic acid, cryptoxanthin, violaxanthin, or zeaxanthin production, respectively; and/or
(j) selecting of non-human organisms with especially high activity of the proteins of the invention from natural or from mutagenized resources and breeding them into the target organism, e.g. the elite crops; and/or
(k) directing a protein encoded by the nucleic acid molecule of the invention or of the polypeptide of the present invention having herein-mentioned activity selected from the group consisting of said activities mentioned in (a) and conferring a abscisic acid, cryptoxanthin, violaxanthin, or zeaxanthin increasing activity, respectively, e.g. of a polypeptide having the activity of a protein as indicated in the respective line in Table II, application no. 13, columns 5 or 8, or its homologs or fragments, if for the corresponding nucleic acid molecule in the respective line in column 6 of Table I the term “plastidic” is indicated, to the plastids by the addition of a plastidial targeting sequence or if for the corresponding nucleic acid molecule in the respective line in column 6 of Table I the term “mitochondric” is indicated, to the mitochondria by the addition of a mitochondrial targeting sequence; and/or
(l) generating the expression of a protein encoded by the nucleic acid molecule of the invention or of the polypeptide of the present invention having herein-mentioned activity selected from the group consisting of said activities mentioned in (a) and conferring a abscisic acid, cryptoxanthin, violaxanthin, or zeaxanthin increasing activity, respectively, e.g. of a polypeptide having the activity of a protein as indicated in the respective line in Table II, application no. 13, columns 5 or 8, or its homologs or fragments, if for the corresponding nucleic acid molecule in the respective line in column 6 of Table I the term “plastidic” or “mitochondric” is indicated, in these organelles by the stable or transient transformation, advantageously stable transformation, of organelles, preferably plastids or mitochondria, with an inventive nucleic acid sequence preferably in form of an expression cassette containing said sequence leading to the expression of the nucleic acids or polypeptides of the invention in the respective organelle; and/or
(m) generating the expression of a protein encoded by the nucleic acid molecule of the invention or of the polypeptide of the present invention having herein-mentioned activity selected from the group consisting of said activities mentioned in (a) and conferring a abscisic acid, cryptoxanthin, violaxanthin, and/or zeaxanthin increasing activity, respectively, e.g. of a polypeptide having the activity of a protein as indicated in the respective line in Table II, application no. 13, columns 5 or 8, or its homologs or fragments, if for the corresponding nucleic acid molecule in the respective line in column 6 of Table I the term “plastidic” or “mitochondric” is indicated, in these organelles by integration of a nucleic acid of the invention into the genome of the respective organelle under control of preferable a promoter selective for the respective organelle.

Preferably, said mRNA is the coding region of the nucleic acid molecule of the present invention and/or the protein conferring the increased expression of a protein encoded by the coding region of the nucleic acid molecule of the present invention alone or linked to a transit nucleic acid sequence or transit peptide encoding nucleic acid sequence or the polypeptide having the herein mentioned activity, e.g. conferring the generation of or increase of abscisic acid, cryptoxanthin, violaxanthin, or zeaxanthin, respectively, after increasing the expression or activity of the encoded polypeptide, non-targeted or in organelles such as plastids and/or mitochondria, preferably plastids, or having the activity of a polypeptide having an activity as the protein as shown in the respective line in Table II, application no. 13, column 3, or its homologs. Preferably the increase of abscisic acid, cryptoxanthin, violaxanthin, or zeaxanthin, respectively, takes place non-targeted or in plastids and/or mitochondria, preferably non-targeted or in plastids.

[0113.1.7.13] to [0122.1.7.13] for the disclosure of these paragraphs see [0113.1.7.7] to [0122.1.7.7] above

The activation of an endogenous polypeptide having above-mentioned activity, e.g. having the activity of a protein as shown in the respective line in Table II, application no. 13, column 3 or of the polypeptide of the invention, e.g. conferring the increase of the fine chemical abscisic acid, cryptoxanthin, violaxanthin, or zeaxanthin, respectively, by increase of expression or activity in the cytoplasm, and/or in the cytosol, and/or in an organelle, such as plastids or mitochondria, can also be increased by introducing a synthetic transcription factor, which binds close to the coding region of the gene encoding the protein as shown in the respective line in Table II, application no. 13, column 5 or 8, or homologs or fragments thereof, and activates its transcription. A chimeric zinc finger protein can be constructed, which comprises a specific DNA-binding domain and an activation domain as e.g. the VP16 domain of Herpes Simplex virus. The specific binding domain can bind to the regulatory region of the gene encoding the protein as shown in the respective line in Table II, application no. 13, column 5 or 8, or homologs or fragments thereof. The expression of the chimeric transcription factor in a non-human organism, in particular in a plant, leads to a specific expression of the protein as shown in the respective line in Table II, application no. 13, column 5 or 8, or homologs or fragments thereof, see e.g. in WO01/52620, Oriz, Proc. Natl. Acad. Sci. USA 99, 13290 (2002), or Guan, Proc. Natl. Acad. Sci. USA 99, 13296 (2002).

[0124.1.7.13] to [0127.1.7.13] for the disclosure of these paragraphs see [0124.1.7.7] to [0127.1.7.7] above

Owing to the introduction of a gene or a plurality of genes conferring the expression of the nucleic acid molecule of the invention or the polypeptide of the invention, for example the nucleic acid construct mentioned below, or encoding the protein as shown in the respective line in Table II, application no. 13, column 5 or 8, or homologs or fragments thereof, into a non-human organism alone or in combination with other genes, it is possible not only to increase the biosynthetic flux towards the end product, but also to increase, modify or create de novo an advantageous, preferably novel metabolites composition in the non-human organism, e.g. an advantageous composition comprising a higher content of (from a viewpoint of nutritional physiology limited) abscisic acid, cryptoxanthin, violaxanthin, or zeaxanthin and if desired other carotenoids, and/or other metabolites, in free or bound form.

for the disclosure of this paragraph see [0129.1.7.7] above

Accordingly, in one embodiment, the process according to the invention relates to a process, which comprises:
(a) providing a non-human organism, preferably a microorganism, a plant cell, a plant tissue, a plant or a part thereof, more preferably a microorganism, a plant tissue, a plant or a part thereof;
(b) increasing the activity of a protein as shown in the respective line in Table II, application no. 13, column 3 or of a polypeptide being encoded by the respective nucleic acid molecule of the present invention and described below, e.g. conferring an increase of the fine chemical abscisic acid, cryptoxanthin, violaxanthin, or zeaxanthin, respectively, in the non-human organism, preferably in the microorganism, the plant cell, the plant tissue, the plant or a part thereof, more preferably a microorganism, a plant tissue, a plant or a part thereof, especially cytoplasmic or in an organelle, like plastids or mitochondria,
(c) growing the non-human organism, preferably the microorganism, the plant cell, the plant tissue, the plant or a part thereof, under conditions which permit the production of said chemical in the non-human organism, preferably the microorganism, the plant cell, the plant tissue, the plant or a part thereof; and
(d) if desired, recovering, optionally isolating, said fine chemical, in free and/or bound form, and, optionally further free and/or bound carotenoids, and/or other metabolites synthesized by the non-human organism, the microorganism, the plant cell, the plant tissue, the plant or a part thereof.

for the disclosure of this paragraph see [0131.1.7.7] above

for the disclosure of this paragraph see [0132.1.7.7] above

for the disclosure of this paragraph see [0133.1.7.7] above

The non-human organism, in particular the microorganism, plant or part thereof, is advantageously grown in such a way that it is not only possible to recover, if desired to isolate the free or bound fine chemical or the free and bound fine chemical but as option it is also possible to produce, recover and, if desired to isolate, other free or/and bound carotenoids, in particular xanthophylls.

The organism such as microorganisms or plants or the recovered, and if desired isolated, the respective fine chemical can then be processed further directly into foodstuffs or animal feeds or for other applications, for example according to the disclosures made in U.S. Pat. No. 6,380,442; U.S. Pat. No. U.S. Pat. No. 6,329,557, U.S. Pat. Nos. 6,329,432, 6,316,012, U.S. Pat. No. 6,309,883; U.S. Pat. No. 6,291,533, U.S. Pat. No. 6,291,294, U.S. Pat. No. 6,287,615, U.S. Pat. No. 6,262,284, U.S. Pat. No. 6,262,284, U.S. Pat. No. 6,261,622, U.S. Pat. No. 6,261,598, U.S. Pat. No. 6,235,315, U.S. Pat. No. 6,224,876, U.S. Pat. No. 6,221,417, U.S. Pat. No. 6,221,412, U.S. Pat. No. 6,218,436, U.S. Pat. No. 6,207,409, U.S. Pat. No. 6,319,872, U.S. Pat. No. 6,132,790, U.S. Pat. No. 6,124,113 U.S. Pat. No. 6,110,478, U.S. Pat. No. 6,093,348, U.S. Pat. No. 6,087,152, or U.S. Pat. No. 6,056,962. (Pharmaceutical and other compositions comprising zeaxanthin are e.g. described in: U.S. Pat. Nos. 6,383,523, 6,368,621, U.S. Pat. No. 6,362,221, U.S. Pat. No. 6,348,200, U.S. Pat. No. 6,316,012: Cosmetic or pharmaceutical composition comprising, in combination, a peroxidase and an anti-singlet oxygen agent, U.S. Pat. No. 6,296,880: Pharmaceutical compositions and methods for managing skin conditions, U.S. Pat. No. 6,296,877: Stable, aqueous dispersions and stable, water-dispersible dry xanthophyll powder, their production and use, U.S. Pat. No. 6,261,598: Carotenoid formulations, comprising a mixture of Bcarotens, lycopene and lutein, U.S. Pat. No. 6,248,378: Enhanced food products, U.S. Pat. No. 6,248,374: Stabilized food additive. Processes for the isolation are described e.g. in U.S. Pat. No. 6,380,442, U.S. Pat. No. 6,362,221: Compositions containing natural lycopene and natural tocopherol, U.S. Pat. No. 6,291,204: Fermentative carotenoid production, U.S. Pat. No. 6,262,284: Process for extraction and purification of lutein, zeaxanthin and rare carotenoids from marigold flowers and plants, or U.S. Pat. No. 6,224,876: Isolation and formulations of nutrient-rich carotenoids. The cited literature describe some preferred embodiments. Said applications describe some advantageous embodiments without meant to be limiting. The fermentation broth, fermentation products, plants or plant products can be purified as described in above mentioned applications and other methods known to the person skilled in the art, e.g. as described in Methods in Enzymology: Carotenoids, Part A: Chemistry, Separation, Quantitation and Antioxidation, by John N Abelson or Part B, Metabolism, Genetics, and Biocehmestry, or described herein below. Products of these different work-up procedures are xanthophylls, in particular zeaxanthin or beta-cryptoxanthin or xanthophylls, in particular zeaxanthin or cryptoxanthin comprising compositions which still comprise fermentation broth, plant particles and cell components in different amounts, advantageously in the range of from 0 to 99% by weight, preferably below 80% by weight, especially preferably between below 50% by weight.

[0135.1.7.13] to [0139.1.7.13] for the disclosure of these paragraphs see [0135.1.7.7] to [0139.1.7.7] above

In another embodiment, the invention relates to a (isolated) nucleic acid molecule, which comprises a nucleic acid molecule selected from the group consisting of:
(a) a nucleic acid molecule encoding a polypeptide shown in Table II, application no. 13, column 5 or 8, preferably shown in Table II B, application no. 13, column 8, or a homolog or a fragment thereof;
(b) a nucleic acid molecule shown in Table I, application no. 13, column 5 or 8, preferably shown in Table I B, application no. 13, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 13, preferably in column 8 of Table II B, application no. 13;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 13, preferably shown in column 8 of Table I B, application no. 13, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 13;
(i) a nucleic acid molecule encoding a polypeptide having the activity represented by a respective protein as depicted in column 5 of Table II, application no. 13;
(j) a nucleic acid molecule comprising a polynucleotide which is obtained by amplifying a cDNA library or a genomic library using the primers in Table III, application no. 13, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 13.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II application no. 13 and confers a generation or an increase of the respective fine chemical in a non-human organism or a part thereof as compared to a corresponding wild-type non-human organism or part thereof.

In another embodiment, the invention relates to a (isolated) nucleic acid molecule, which comprises a nucleic acid molecule selected from the group consisting of:
(a) a nucleic acid molecule encoding a polypeptide shown in Table II, column 5 or 8, application no. 13, preferably shown in Table II A, application no. 13, in column 5 or in Table II A, application no. 13, column 8 or in Table II B, application no. 13, column 8, or a homolog or a fragment thereof;
(b) a nucleic acid molecule shown in Table I, application no. 13, column 5 or 8, preferably shown in Table I A, application no. 13, in column 5 or in Table I A, application no. 13, column 8 or in Table I B, application no. 13, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 13, preferably shown in Table II A, application no. 13, in column 5 or in Table II A, application no. 13, column 8 or in Table II B, application no. 13, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 13, preferably shown in Table I A, application no. 13, in column 5 or in Table I A, application no. 13, column 8 or in Table I B column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 13;
(i) a nucleic acid molecule encoding a polypeptide having the activity represented by a respective protein as depicted in column 5 of Table II, application no. 13;
(j) a nucleic acid molecule comprising a polynucleotide which is obtained by amplifying a cDNA library or a genomic library using the primers in Table III, application no. 13, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt,500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto;
   whereby, preferably, the nucleic acid molecule according to (a), (b), (c), (d), (e), (f), (g), (h), (i), (j) or (k) distinguishes over the sequence depicted in Table I A and/or I B, application no. 13, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 13, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 13, column 5 or 8, or the coding region thereof, respectively. In a further embodiment the nucleic acid molecule does not encode the polypeptide sequence shown in Table II A and/or II B, application no. 13, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 13, column 5 or 8 and does not encode a protein of the sequence shown in Table II A and/or II B, application no. 13, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 13, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 13, column 5 or 8 but less than 100%, preferably less than 99.999%, 99.99%, 99.9%, 99%, 98%, 97%, 96% or 95% identical to the sequence shown in Table II A and/or II B, application no. 13, column 5 or 8.
   (All the references to Tables I to IV are references to the corresponding lines of Tables I to IV.) [0143.1.13.13] In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 13.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II, application no. 13, and confers a generation or an increase of the respective fine chemical in a non-human organism or a part thereof as compared to a corresponding wild-type non-human organism or part thereof.

[0144.1.7.13] to [0155.1.7.13] for the disclosure of these paragraphs see [0144.1.7.7] to [0155.1.7.7] above

In a preferred embodiment a nucleic acid construct, for example an expression cassette, comprises upstream, i.e. at the 5′ end of the encoding sequence, a promoter and downstream, i.e. at the 3′ end, a polyadenylation signal and optionally other regulatory elements which are operably linked to the intervening encoding sequence with one of the nucleic acids as depicted in the respective line in Table I, application no. 13, column 5 or 8, preferably the coding region thereof, or a homolog or a fragment thereof. By an operable linkage is meant the sequential arrangement of promoter, encoding sequence, terminator, optionally other regulatory elements and optionally targeting sequences in such a way that each of the regulatory elements/targeting sequence can fulfill its function in the expression of the encoding sequence in due manner. In one embodiment the sequences preferred for operable linkage are targeting sequences for ensuring subcellular localization in organelles, like plastids or mitochondria. However, targeting sequences for ensuring subcellular localization in the endoplasmic reticulum (=ER), in the nucleus, in oil corpuscles or other compartments may also be employed as well as translation promoters such as the 5′ lead sequence in tobacco mosaic virus (Gallie et al., Nucl. Acids Res. 15, 8693 (1987)).

for the disclosure of this paragraph see [0157.1.7.7] above

In an embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 13, column 5 or 8, preferably shown in Table II A, application no. 13, column 5, or in Table II A, application no. 13, column 8, or in Table II B, application no. 13, column 8, or a homolog or a fragment thereof;
   (b) a nucleic acid molecule shown in Table I, application no. 13, column 5 or 8, preferably shown in Table I A, application no. 13, column 5, or in Table I A, application no. 13, column 8, or in Table I B, application no. 13, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 13, preferably shown in Table II A, application no. 13, column 5, or in Table II A, application no. 13, column 8, or in Table II B, application no. 13, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 13, preferably shown in Table I A, application no. 13, column 5, or in Table I A, application no. 13, column 8, or in Table I B, application no. 13, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 13;
   (i) a nucleic acid molecule encoding a polypeptide having the activity represented by a respective protein as depicted in column 5 of Table II, application no. 13;
   (j) a nucleic acid molecule comprising a polynucleotide which is obtained by amplifying a cDNA library or a genomic library using the primers in Table III, application no.

13, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
or a nucleic acid molecule comprising a sequence which is complementary thereto.
   and, optionally
3) nucleic acid molecule encoding a transit peptide, preferably encoding a plastidal transit peptide or a mitochondrial transit peptide;
   which are operable linked.
   (All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 13.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 13 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 13, in the same line as of the respective nucleic acid molecule according to 2) “plastidic” is depicted, or encoding a mitochondrial transit peptide, especially in case in column 6 of Table I, application no. 13, in the same line as of the respective nucleic acid molecule according to 2) “mitochondric” is depicted.

In another embodiment thereof the expression cassette does not comprise 3) a nucleic acid molecule encoding a transit peptide, especially in case in column 6 of Table I, application no. 13, in the same line as of the respective nucleic acid molecule according to 2) “non-targeted” is depicted.

In a further embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 13, column 5 or 8, preferably shown in Table II A, application no. 13, column 5, or in Table II A, application no. 13, column 8, or in Table II B, application no. 13, column 8, or a homolog or a fragment thereof;
   (b) a nucleic acid molecule shown in Table I, application no. 13, column 5 or 8, preferably shown in Table I A, application no. 13, column 5, or in Table I A, application no. 13, column 8, or in Table I B, application no. 13, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 13, preferably shown in Table II A, application no. 13, column 5, or in Table II A, application no. 13, column 8, or in Table II B, application no. 13, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 13, preferably shown in Table I A, application no. 13, column 5, or in Table I A, application no. 13, column 8,or in Table I B, application no. 13, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 13;
   (i) a nucleic acid molecule encoding a polypeptide having the activity represented by a respective protein as depicted in column 5 of Table II, application no. 13;
   (j) a nucleic acid molecule comprising a polynucleotide which is obtained by amplifying a cDNA library or a genomic library using the primers in Table III, application no. 13, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, optionally
3) nucleic acid molecule encoding a transit peptide, preferably encoding a plastidal transit peptide or a mitochondrial transit peptide;
   which are operable linked and
   whereby, preferably, the nucleic acid molecule according to 2 (a), (b), (c), (d), (e), (f), (g), (h), (i), (j) or (k) distinguishes over the sequence depicted in Table I A and/or I B, application no. 13, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention according to 2) does not consist of the sequence shown in Table I A and/or I B, application no. 13, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 13, column 5 or 8, or the coding region thereof, respectively. In a further embodiment the nucleic acid molecule does not encode the polypeptide sequence shown in Table II A and/or II B, application no. 13, column 5 or 8 but homologs thereof.

Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 13, column 5 or 8 and does not encode a protein of the sequence shown in Table II A and/or II B, application no. 13, column 5 or 8. Accordingly, in one embodiment, the protein encoded by a sequence of a nucleic acid according to 2(a), (b), (c), (d), (e), (f), (g), (h), (i), (j) or (k) does not consist of the sequence shown in Table I A and/or I B, application no. 13, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 13, column 5 or 8 but less than 100%, 99.999%, 99.99%, 99.9%, 99%, 98%, 97%, 96% or 95% identical to the sequence shown in Table II A and/or II B, application no. 13, column 5 or 8.

(All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 13.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 13 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 13, in the same line as of the respective nucleic acid molecule according to 2) “plastidic” is depicted, or encoding a mitochondrial transit peptide, especially in case in column 6 of Table I, application no. 13, in the same line as of the respective nucleic acid molecule according to 2) “mitochondric” is depicted.

In another embodiment thereof the expression cassette does not comprise 3) a nucleic acid molecule encoding a transit peptide, especially in case in column 6 of Table I, application no. 13, in the same line as of the respective nucleic acid molecule according to 2) “non-targeted” is depicted.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 130491, or a homolog or fragment thereof, or a homolog thereof being depicted in Table I, application no. 13, column 8, in the same line as SEQ ID NO. 130491, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 130491 is depicted, plastidic is mentioned, or a mitochondrial transit peptide in case in Table I, application no. 13, in column 6 in the same line as SEQ ID NO. 130491 is depicted, mitochondric is mentioned; which is operable linked, too.

In another embodiment thereof the expression cassette does not comprise an additional transit peptide in case in Table I, application no. 13, in column 6 in the same line as SEQ ID NO. 130491 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 120952, or a homolog or fragment thereof, or a homolog thereof being depicted in Table I, application no. 13, column 8, in the same line as SEQ ID NO. 120952, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 120952 is depicted, plastidic is mentioned, or a mitochondrial transit peptide in case in Table I, application no. 13, in column 6 in the same line as SEQ ID NO. 120952 is depicted, mitochondric is mentioned; which is operable linked, too.

In another embodiment thereof the expression cassette does not comprise an additional transit peptide in case in Table I, application no. 13, in column 6 in the same line as SEQ ID NO. 120952 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 2573, or a homolog or fragment thereof, or a homolog thereof being depicted in Table I, application no. 13, column 8, in the same line as SEQ ID NO. 2573, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 2573 is depicted, plastidic is mentioned, or a mitochondrial transit peptide in case in Table I, application no. 13, in column 6 in the same line as SEQ ID NO. 2573 is depicted, mitochondric is mentioned; which is operable linked, too.

In another embodiment thereof the expression cassette does not comprise an additional transit peptide in case in Table I, application no. 13, in column 6 in the same line as SEQ ID NO. 2573 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 23482, or a homolog or fragment thereof, or a homolog thereof being depicted in Table I, application no. 13, column 8, in the same line as SEQ ID NO. 23482, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 23482 is depicted, plastidic is mentioned, or a mitochondrial transit peptide in case in Table I, application no. 13, in column 6 in the same line as SEQ ID NO. 23482 is depicted, mitochondric is mentioned; which is operable linked, too.

In another embodiment thereof the expression cassette does not comprise an additional transit peptide in case in Table I, application no. 13, in column 6 in the same line as SEQ ID NO. 23482 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 3279, or a homolog or fragment thereof, or a homolog thereof being depicted in Table I, application no. 13, column 8, in the same line as SEQ ID NO. 3279, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 3279 is depicted, plastidic is mentioned, or a mitochondrial transit peptide in case in Table I, application no. 13, in column 6 in the same line as SEQ ID NO. 3279 is depicted, mitochondric is mentioned; which is operable linked, too.

In another embodiment thereof the expression cassette does not comprise an additional transit peptide in case in Table I, application no. 13, in column 6 in the same line as SEQ ID NO. 3279 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68849, or a homolog or fragment thereof, or a homolog thereof being depicted in Table I, application no. 13, column 8, in the same line as SEQ ID NO. 68849, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 68849 is depicted, plastidic is mentioned, or a mitochondrial transit peptide in case in Table I, application no. 13, in column 6 in the same line as SEQ ID NO. 68849 is depicted, mitochondric is mentioned; which is operable linked, too.

In another embodiment thereof the expression cassette does not comprise an additional transit peptide in case in Table I, application no. 13, in column 6 in the same line as SEQ ID NO. 68849 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4102, or a homolog or fragment thereof, or a homolog thereof being depicted in Table I, application no. 13, column 8, in the same line as SEQ ID NO. 4102, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 4102 is depicted, plastidic is mentioned, or a mitochondrial transit peptide in case in Table I, application no. 13, in column 6 in the same line as SEQ ID NO. 4102 is depicted, mitochondric is mentioned; which is operable linked, too.

In another embodiment thereof the expression cassette does not comprise an additional transit peptide in case in Table I, application no. 13, in column 6 in the same line as SEQ ID NO. 4102 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 102899, or a homolog or fragment thereof, or a homolog thereof being depicted in Table I, application no. 13, column 8, in the same line as SEQ ID NO. 102899, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 102899 is depicted, plastidic is mentioned, or a mitochondrial transit peptide in case in Table I, application no. 13, in column 6 in the same line as SEQ ID NO. 102899 is depicted, mitochondric is mentioned; which is operable linked, too.

In another embodiment thereof the expression cassette does not comprise an additional transit peptide in case in Table I, application no. 13, in column 6 in the same line as SEQ ID NO. 102899 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 102941, or a homolog or fragment thereof, or a homolog thereof being depicted in Table I, application no. 13, column 8, in the same line as SEQ ID NO. 102941, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 102941 is depicted, plastidic is mentioned, or a mitochondrial transit peptide in case in Table I, application no. 13, in column 6 in the same line as SEQ ID NO. 102941 is depicted, mitochondric is mentioned; which is operable linked, too.

In another embodiment thereof the expression cassette does not comprise an additional transit peptide in case in Table I, application no. 13, in column 6 in the same line as SEQ ID NO. 102941 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 34301, or a homolog or fragment thereof, or a homolog thereof being depicted in Table I, application no. 13, column 8, in the same line as SEQ ID NO. 34301, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 34301 is depicted, plastidic is mentioned, or a mitochondrial transit peptide in case in Table I, application no. 13, in column 6 in the same line as SEQ ID NO. 34301 is depicted, mitochondric is mentioned; which is operable linked, too.

In another embodiment thereof the expression cassette does not comprise an additional transit peptide in case in Table I, application no. 13, in column 6 in the same line as SEQ ID NO. 34301 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7941, or a homolog or fragment thereof, or a homolog thereof being depicted in Table I, application no. 13, column 8, in the same line as SEQ ID NO. 7941, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 7941 is depicted, plastidic is mentioned, or a mitochondrial transit peptide in case in Table I, application no. 13, in column 6 in the same line as SEQ ID NO. 7941 is depicted, mitochondric is mentioned; which is operable linked, too.

In another embodiment thereof the expression cassette does not comprise an additional transit peptide in case in Table I, application no. 13, in column 6 in the same line as SEQ ID NO. 7941 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 36880, or a homolog or fragment thereof, or a homolog thereof being depicted in Table I, application no. 13, column 8, in the same line as SEQ ID NO. 36880, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 36880 is depicted, plastidic is mentioned, or a mitochondrial transit peptide in case in Table I, application no. 13, in column 6 in the same line as SEQ ID NO. 36880 is depicted, mitochondric is mentioned; which is operable linked, too.

In another embodiment thereof the expression cassette does not comprise an additional transit peptide in case in Table I, application no. 13, in column 6 in the same line as SEQ ID NO. 36880 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 8316, or a homolog or fragment thereof, or a homolog thereof being depicted in Table I, application no. 13, column 8, in the same line as SEQ ID NO. 8316, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 8316 is depicted, plastidic is mentioned, or a mitochondrial transit peptide in case in Table I, application no. 13, in column 6 in the same line as SEQ ID NO. 8316 is depicted, mitochondric is mentioned; which is operable linked, too.

In another embodiment thereof the expression cassette does not comprise an additional transit peptide in case in Table I, application no. 13, in column 6 in the same line as SEQ ID NO. 8316 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 8920, or a homolog or fragment thereof, or a homolog thereof being depicted in Table I, application no. 13, column 8, in the same line as SEQ ID NO. 8920, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 8920 is depicted, plastidic is mentioned, or a mitochondrial transit peptide in case in Table I, application no. 13, in column 6 in the same line as SEQ ID NO. 8920 is depicted, mitochondric is mentioned; which is operable linked, too.

In another embodiment thereof the expression cassette does not comprise an additional transit peptide in case in Table I, application no. 13, in column 6 in the same line as SEQ ID NO. 8920 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115595, or a homolog or fragment thereof, or a homolog thereof being depicted in Table I, application no. 13, column 8, in the same line as SEQ ID NO. 115595, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 115595 is depicted, plastidic is mentioned, or a mitochondrial transit peptide in case in Table I, application no. 13, in column 6 in the same line as SEQ ID NO. 115595 is depicted, mitochondric is mentioned; which is operable linked, too.

In another embodiment thereof the expression cassette does not comprise an additional transit peptide in case in Table I, application no. 13, in column 6 in the same line as SEQ ID NO. 115595 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39013, or a homolog or fragment thereof, or a homolog thereof being depicted in Table I, application no. 13, column 8, in the same line as SEQ ID NO. 39013, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 39013 is depicted, plastidic is mentioned, or a mitochondrial transit peptide in case in Table I, application no. 13, in column 6 in the same line as SEQ ID NO. 39013 is depicted, mitochondric is mentioned; which is operable linked, too.

In another embodiment thereof the expression cassette does not comprise an additional transit peptide in case in Table I, application no. 13, in column 6 in the same line as SEQ ID NO. 39013 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40329, or a homolog or fragment thereof, or a homolog thereof being depicted in Table I, application no. 13, column 8, in the same line as SEQ ID NO. 40329, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 40329 is depicted, plastidic is mentioned, or a mitochondrial transit peptide in case in Table I, application no. 13, in column 6 in the same line as SEQ ID NO. 40329 is depicted, mitochondric is mentioned; which is operable linked, too.

In another embodiment thereof the expression cassette does not comprise an additional transit peptide in case in Table I, application no. 13, in column 6 in the same line as SEQ ID NO. 40329 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40741, or a homolog or fragment thereof, or a homolog thereof being depicted in Table I, application no. 13, column 8, in the same line as SEQ ID NO. 40741, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 40741 is depicted, plastidic is mentioned, or a mitochondrial transit peptide in case in Table I, application no. 13, in column 6 in the same line as SEQ ID NO. 40741 is depicted, mitochondric is mentioned; which is operable linked, too.

In another embodiment thereof the expression cassette does not comprise an additional transit peptide in case in Table I, application no. 13, in column 6 in the same line as SEQ ID NO. 40741 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115719, or a homolog or fragment thereof, or a homolog thereof being depicted in Table I, application no. 13, column 8, in the same line as SEQ ID NO. 115719, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 115719 is depicted, plastidic is mentioned, or a mitochondrial transit peptide in case in Table I, application no. 13, in column 6 in the same line as SEQ ID NO. 115719 is depicted, mitochondric is mentioned; which is operable linked, too.

In another embodiment thereof the expression cassette does not comprise an additional transit peptide in case in Table I, application no. 13, in column 6 in the same line as SEQ ID NO. 115719 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 41073, or a homolog or fragment thereof, or a homolog thereof being depicted in Table I, application no. 13, column 8, in the same line as SEQ ID NO. 41073, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 41073 is depicted, plastidic is mentioned, or a mitochondrial transit peptide in case in Table I, application no. 13, in column 6 in the same line as SEQ ID NO. 41073 is depicted, mitochondric is mentioned; which is operable linked, too.

In another embodiment thereof the expression cassette does not comprise an additional transit peptide in case in Table I, application no. 13, in column 6 in the same line as SEQ ID NO. 41073 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 9492, or a homolog or fragment thereof, or a homolog thereof being depicted in Table I, application no. 13, column 8, in the same line as SEQ ID NO. 9492, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 9492 is depicted, plastidic is mentioned, or a mitochondrial transit peptide in case in Table I, application no. 13, in column 6 in the same line as SEQ ID NO. 9492 is depicted, mitochondric is mentioned; which is operable linked, too.

In another embodiment thereof the expression cassette does not comprise an additional transit peptide in case in Table I, application no. 13, in column 6 in the same line as SEQ ID NO. 9492 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 10104, or a homolog or fragment thereof, or a homolog thereof being depicted in Table I, application no. 13, column 8, in the same line as SEQ ID NO. 10104, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 10104 is depicted, plastidic is mentioned, or a mitochondrial transit peptide in case in Table I, application no. 13, in column 6 in the same line as SEQ ID NO. 10104 is depicted, mitochondric is mentioned; which is operable linked, too.

In another embodiment thereof the expression cassette does not comprise an additional transit peptide in case in Table I, application no. 13, in column 6 in the same line as SEQ ID NO. 10104 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 94386, or a homolog or fragment thereof, or a homolog thereof being depicted in Table I, application no. 13, column 8, in the same line as SEQ ID NO. 94386, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 94386 is depicted, plastidic is mentioned, or a mitochondrial transit peptide in case in Table I, application no. 13, in column 6 in the same line as SEQ ID NO. 94386 is depicted, mitochondric is mentioned; which is operable linked, too.

In another embodiment thereof the expression cassette does not comprise an additional transit peptide in case in Table I, application no. 13, in column 6 in the same line as SEQ ID NO. 94386 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 10708, or a homolog or fragment thereof, or a homolog thereof being depicted in Table I, application no. 13, column 8, in the same line as SEQ ID NO. 10708, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 10708 is depicted, plastidic is mentioned, or a mitochondrial transit peptide in case in Table I, application no. 13, in column 6 in the same line as SEQ ID NO. 10708 is depicted, mitochondric is mentioned; which is operable linked, too.

In another embodiment thereof the expression cassette does not comprise an additional transit peptide in case in Table I, application no. 13, in column 6 in the same line as SEQ ID NO. 10708 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 105659, or a homolog or fragment thereof, or a homolog thereof being depicted in Table I, application no. 13, column 8, in the same line as SEQ ID NO. 105659, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 105659 is depicted, plastidic is mentioned, or a mitochondrial transit peptide in case in Table I, application no. 13, in column 6 in the same line as SEQ ID NO. 105659 is depicted, mitochondric is mentioned; which is operable linked, too.

In another embodiment thereof the expression cassette does not comprise an additional transit peptide in case in Table I, application no. 13, in column 6 in the same line as SEQ ID NO. 105659 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 44378, or a homolog or fragment thereof, or a homolog thereof being depicted in Table I, application no. 13, column 8, in the same line as SEQ ID NO. 44378, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 44378 is depicted, plastidic is mentioned, or a mitochondrial transit peptide in case in Table I, application no. 13, in column 6 in the same line as SEQ ID NO. 44378 is depicted, mitochondric is mentioned; which is operable linked, too.

In another embodiment thereof the expression cassette does not comprise an additional transit peptide in case in Table I, application no. 13, in column 6 in the same line as SEQ ID NO. 44378 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 130673, or a homolog or fragment thereof, or a homolog thereof being depicted in Table I, application no. 13, column 8, in the same line as SEQ ID NO. 130673, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 130673 is depicted, plastidic is mentioned, or a mitochondrial transit peptide in case in Table I, application no. 13, in column 6 in the same line as SEQ ID NO. 130673 is depicted, mitochondric is mentioned; which is operable linked, too.

In another embodiment thereof the expression cassette does not comprise an additional transit peptide in case in Table I, application no. 13, in column 6 in the same line as SEQ ID NO. 130673 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 45556, or a homolog or fragment thereof, or a homolog thereof being depicted in Table I, application no. 13, column 8, in the same line as SEQ ID NO. 45556, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 45556 is depicted, plastidic is mentioned, or a mitochondrial transit peptide in case in Table I, application no. 13, in column 6 in the same line as SEQ ID NO. 45556 is depicted, mitochondric is mentioned; which is operable linked, too.

In another embodiment thereof the expression cassette does not comprise an additional transit peptide in case in Table I, application no. 13, in column 6 in the same line as SEQ ID NO. 45556 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 46751, or a homolog or fragment thereof, or a homolog thereof being depicted in Table I, application no. 13, column 8, in the same line as SEQ ID NO. 46751, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 46751 is depicted, plastidic is mentioned, or a mitochondrial transit peptide in case in Table I, application no. 13, in column 6 in the same line as SEQ ID NO. 46751 is depicted, mitochondric is mentioned; which is operable linked, too.

In another embodiment thereof the expression cassette does not comprise an additional transit peptide in case in Table I, application no. 13, in column 6 in the same line as SEQ ID NO. 46751 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 53189, or a homolog or fragment thereof, or a homolog thereof being depicted in Table I, application no. 13, column 8, in the same line as SEQ ID NO. 53189, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 53189 is depicted, plastidic is mentioned, or a mitochondrial transit peptide in case in Table I, application no. 13, in column 6 in the same line as SEQ ID NO. 53189 is depicted, mitochondric is mentioned; which is operable linked, too.

In another embodiment thereof the expression cassette does not comprise an additional transit peptide in case in Table I, application no. 13, in column 6 in the same line as SEQ ID NO. 53189 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 58823, or a homolog or fragment thereof, or a homolog thereof being depicted in Table I, application no. 13, column 8, in the same line as SEQ ID NO. 58823, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 58823 is depicted, plastidic is mentioned, or a mitochondrial transit peptide in case in Table I, application no. 13, in column 6 in the same line as SEQ ID NO. 58823 is depicted, mitochondric is mentioned; which is operable linked, too.

In another embodiment thereof the expression cassette does not comprise an additional transit peptide in case in Table I, application no. 13, in column 6 in the same line as SEQ ID NO. 58823 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 63167, or a homolog or fragment thereof, or a homolog thereof being depicted in Table I, application no. 13, column 8, in the same line as SEQ ID NO. 63167, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 63167 is depicted, plastidic is mentioned, or a mitochondrial transit peptide in case in Table I, application no. 13, in column 6 in the same line as SEQ ID NO. 63167 is depicted, mitochondric is mentioned; which is operable linked, too.

In another embodiment thereof the expression cassette does not comprise an additional transit peptide in case in Table I, application no. 13, in column 6 in the same line as SEQ ID NO. 63167 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 14706, or a homolog or fragment thereof, or a homolog thereof being depicted in Table I, application no. 13, column 8, in the same line as SEQ ID NO. 14706, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 14706 is depicted, plastidic is mentioned, or a mitochondrial transit peptide in case in Table I, application no. 13, in column 6 in the same line as SEQ ID NO. 14706 is depicted, mitochondric is mentioned; which is operable linked, too.

In another embodiment thereof the expression cassette does not comprise an additional transit peptide in case in Table I, application no. 13, in column 6 in the same line as SEQ ID NO. 14706 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 90278, or a homolog or fragment thereof, or a homolog thereof being depicted in Table I, application no. 13, column 8, in the same line as SEQ ID NO. 90278, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 90278 is depicted, plastidic is mentioned, or a mitochondrial transit peptide in case in Table I, application no. 13, in column 6 in the same line as SEQ ID NO. 90278 is depicted, mitochondric is mentioned; which is operable linked, too.

In another embodiment thereof the expression cassette does not comprise an additional transit peptide in case in Table I, application no. 13, in column 6 in the same line as SEQ ID NO. 90278 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 130742, or a homolog or fragment thereof, or a homolog thereof being depicted in Table I, application no. 13, column 8, in the same line as SEQ ID NO. 130742, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 130742 is depicted, plastidic is mentioned, or a mitochondrial transit peptide in case in Table I, application no. 13, in column 6 in the same line as SEQ ID NO. 130742 is depicted, mitochondric is mentioned; which is operable linked, too.

In another embodiment thereof the expression cassette does not comprise an additional transit peptide in case in Table I, application no. 13, in column 6 in the same line as SEQ ID NO. 130742 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 130903, or a homolog or fragment thereof, or a homolog thereof being depicted in Table I, application no. 13, column 8, in the same line as SEQ ID NO. 130903, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 130903 is depicted, plastidic is mentioned, or a mitochondrial transit peptide in case in Table I, application no. 13, in column 6 in the same line as SEQ ID NO. 130903 is depicted, mitochondric is mentioned; which is operable linked, too.

In another embodiment thereof the expression cassette does not comprise an additional transit peptide in case in Table I, application no. 13, in column 6 in the same line as SEQ ID NO. 130903 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 130932, or a homolog or fragment thereof, or a homolog thereof being depicted in Table I, application no. 13, column 8, in the same line as SEQ ID NO. 130932, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 130932 is depicted, plastidic is mentioned, or a mitochondrial transit peptide in case in Table I, application no. 13, in column 6 in the same line as SEQ ID NO. 130932 is depicted, mitochondric is mentioned; which is operable linked, too.

In another embodiment thereof the expression cassette does not comprise an additional transit peptide in case in Table I, application no. 13, in column 6 in the same line as SEQ ID NO. 130932 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 130986, or a homolog or fragment thereof, or a homolog thereof being depicted in Table I, application no. 13, column 8, in the same line as SEQ ID NO. 130986, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 130986 is depicted, plastidic is mentioned, or a mitochondrial transit peptide in case in Table I, application no. 13, in column 6 in the same line as SEQ ID NO. 130986 is depicted, mitochondric is mentioned; which is operable linked, too.

In another embodiment thereof the expression cassette does not comprise an additional transit peptide in case in Table I, application no. 13, in column 6 in the same line as SEQ ID NO. 130986 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 67646, or a homolog or fragment thereof, or a homolog thereof being depicted in Table I, application no. 13, column 8, in the same line as SEQ ID NO. 67646, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 67646 is depicted, plastidic is mentioned, or a mitochondrial transit peptide in case in Table I, application no. 13, in column 6 in the same line as SEQ ID NO. 67646 is depicted, mitochondric is mentioned; which is operable linked, too.

In another embodiment thereof the expression cassette does not comprise an additional transit peptide in case in Table I, application no. 13, in column 6 in the same line as SEQ ID NO. 67646 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 130713, or a homolog or fragment thereof, or a homolog thereof being depicted in Table I, application no. 13, column 8, in the same line as SEQ ID NO. 130713, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 130713 is depicted, plastidic is mentioned, or a mitochondrial transit peptide in case in Table I, application no. 13, in column 6 in the same line as SEQ ID NO. 130713 is depicted, mitochondric is mentioned; which is operable linked, too.

In another embodiment thereof the expression cassette does not comprise an additional transit peptide in case in Table I, application no. 13, in column 6 in the same line as SEQ ID NO. 130713 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 130999, or a homolog or fragment thereof, or a homolog thereof being depicted in Table I, application no. 13, column 8, in the same line as SEQ ID NO. 130999, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 130999 is depicted, plastidic is mentioned, or a mitochondrial transit peptide in case in Table I, application no. 13, in column 6 in the same line as SEQ ID NO. 130999 is depicted, mitochondric is mentioned; which is operable linked, too.

In another embodiment thereof the expression cassette does not comprise an additional transit peptide in case in Table I, application no. 13, in column 6 in the same line as SEQ ID NO. 130999 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 45394, or a homolog or fragment thereof, or a homolog thereof being depicted in Table I, application no. 13, column 8, in the same line as SEQ ID NO. 45394, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 45394 is depicted, plastidic is mentioned, or a mitochondrial transit peptide in case in Table I, application no. 13, in column 6 in the same line as SEQ ID NO. 45394 is depicted, mitochondric is mentioned; which is operable linked, too.

In another embodiment thereof the expression cassette does not comprise an additional transit peptide in case in Table I, application no. 13, in column 6 in the same line as SEQ ID NO. 45394 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 54337, or a homolog or fragment thereof, or a homolog thereof being depicted in Table I, application no. 13, column 8, in the same line as SEQ ID NO. 54337, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 54337 is depicted, plastidic is mentioned, or a mitochondrial transit peptide in case in Table I, application no. 13, in column 6 in the same line as SEQ ID NO. 54337 is depicted, mitochondric is mentioned; which is operable linked, too.

In another embodiment thereof the expression cassette does not comprise an additional transit peptide in case in Table I, application no. 13, in column 6 in the same line as SEQ ID NO. 54337 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 86447, or a homolog or fragment thereof, or a homolog thereof being depicted in Table I, application no. 13, column 8, in the same line as SEQ ID NO. 86447, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 86447 is depicted, plastidic is mentioned, or a mitochondrial transit peptide in case in Table I, application no. 13, in column 6 in the same line as SEQ ID NO. 86447 is depicted, mitochondric is mentioned; which is operable linked, too.

In another embodiment thereof the expression cassette does not comprise an additional transit peptide in case in Table I, application no. 13, in column 6 in the same line as SEQ ID NO. 86447 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68657, or a homolog or fragment thereof, or a homolog thereof being depicted in Table I, application no. 13, column 8, in the same line as SEQ ID NO. 68657, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 68657 is depicted, plastidic is mentioned, or a mitochondrial transit peptide in case in Table I, application no. 13, in column 6 in the same line as SEQ ID NO. 68657 is depicted, mitochondric is mentioned; which is operable linked, too.

In another embodiment thereof the expression cassette does not comprise an additional transit peptide in case in Table I, application no. 13, in column 6 in the same line as SEQ ID NO. 68657 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 18235, or a homolog or fragment thereof, or a homolog thereof being depicted in Table I, application no. 13, column 8, in the same line as SEQ ID NO. 18235, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 18235 is depicted, plastidic is mentioned, or a mitochondrial transit peptide in case in Table I, application no. 13, in column 6 in the same line as SEQ ID NO. 18235 is depicted, mitochondric is mentioned; which is operable linked, too.

In another embodiment thereof the expression cassette does not comprise an additional transit peptide in case in Table I, application no. 13, in column 6 in the same line as SEQ ID NO. 18235 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 19419, or a homolog or fragment thereof, or a homolog thereof being depicted in Table I, application no. 13, column 8, in the same line as SEQ ID NO. 19419, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 19419 is depicted, plastidic is mentioned, or a mitochondrial transit peptide in case in Table I, application no. 13, in column 6 in the same line as SEQ ID NO. 19419 is depicted, mitochondric is mentioned; which is operable linked, too.

In another embodiment thereof the expression cassette does not comprise an additional transit peptide in case in Table I, application no. 13, in column 6 in the same line as SEQ ID NO. 19419 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 70715, or a homolog or fragment thereof, or a homolog thereof being depicted in Table I, application no. 13, column 8, in the same line as SEQ ID NO. 70715, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 70715 is depicted, plastidic is mentioned, or a mitochondrial transit peptide in case in Table I, application no. 13, in column 6 in the same line as SEQ ID NO. 70715 is depicted, mitochondric is mentioned; which is operable linked, too.

In another embodiment thereof the expression cassette does not comprise an additional transit peptide in case in Table I, application no. 13, in column 6 in the same line as SEQ ID NO. 70715 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 20346, or a homolog or fragment thereof, or a homolog thereof being depicted in Table I, application no. 13, column 8, in the same line as SEQ ID NO. 20346, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 13, in column 6 in the same line as SEQ ID NO. 20346 is depicted, plastidic is mentioned, or a mitochondrial transit peptide in case in Table I, application no. 13, in column 6 in the same line as SEQ ID NO. 20346 is depicted, mitochondric is mentioned; which is operable linked, too.

In another embodiment thereof the expression cassette does not comprise an additional transit peptide in case in Table I, application no. 13, in column 6 in the same line as SEQ ID NO. 20346 is depicted, non-targeted is mentioned.

[0165.1.7.13] to [0170.1.7.13] for the disclosure of these paragraphs see [0165.1.7.7] to [0170.1.7.7] above

The invention further provides a vector, comprising a nucleic acid molecule comprising 1) a nucleic acid molecule selected from the group consisting of:
(a) a nucleic acid molecule encoding a polypeptide shown in Table II, column 5 or 8, application no. 13, preferably shown in Table II A, application no. 13, in column 5, or in Table II A, application no. 13, column 8, or in Table II B, application no. 13, column 8, or a homolog or a fragment thereof;
(b) a nucleic acid molecule shown in Table I, application no. 13, column 5 or 8, preferably shown in Table I A, application no. 13, in column 5, or in Table I A, application no. 13, column 8, or in Table I B, application no. 13, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 13, preferably shown in Table II A, application no. 13, in column 5, or in Table II A, application no. 13, column 8, or in Table II B, application no. 13, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 13, preferably shown in Table I A, application no. 13, in column 5, or in Table I A, application no. 13, column 8, or in Table I B, application no. 13, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 13;
(i) a nucleic acid molecule encoding a polypeptide having the activity represented by a protein as depicted in column 5 of Table II, application no. 13;
(j) a nucleic acid molecule comprising a polynucleotide which is obtained by amplifying a cDNA library or a genomic library using the primers in Table III, application no. 13, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 13.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 13, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 13.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 13, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 13, in the same line as of the respective nucleic acid molecule according to 1) “plastidic” is depicted, or encoding a mitochondrial transit peptide, especially in case in column 6 of Table I, application no. 13, in the same line as of the respective nucleic acid molecule according to 1) “mitochondric” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 13, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 13, in the same line as of the respective nucleic acid molecule according to 1) “plastidic” is depicted, or encoding a mitochondrial transit peptide, especially in case in column 6 of Table I, application no. 13, in the same line as of the respective nucleic acid molecule according to 1) “mitochondric” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operable linked; a nucleic acid molecule encoding a transit peptide, especially in case in column 6 of Table I, application no. 13, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 13, in the same line as of the respective nucleic acid molecule according to 1) “plastidic” is depicted, or encoding a mitochondrial transit peptide, especially in case in column 6 of Table I, application no. 13, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 13 (in the same line).

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 13, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 13.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 13, in the same line as of the respective nucleic acid molecule according to 1) “plastidic” is depicted, or encoding a mitochondrial transit peptide, especially in case in column 6 of Table I, application no. 13, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 13, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 13, in the same line as of the respective nucleic acid molecule according to 1) “plastidic” is depicted, or encoding a mitochondrial transit peptide, especially in case in column 6 of Table I, application no. 13, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 13 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 13, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 13 (in the same line) and confers a generation or an increase of the respective fine chemical in a non-human organism or a part thereof as compared to a corresponding wild-type non-human organism or part thereof.

In a preferred embodiment of said vector said nucleic acid molecule according to 1 (a), (b), (c), (d), (e), (f), (g), (h), (i), (j) or (k), said promoter and optionally said nucleic acid molecule encoding a transit peptide are encompassed in an expression cassette. Accordingly in an embodiment the invention provides a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

The invention further provides a vector comprising a nucleic acid molecule comprising 1) a nucleic acid molecule selected from the group consisting of:
(a) a nucleic acid molecule encoding a polypeptide shown in Table II, column 5 or 8, application no. 13, preferably shown in Table II A, application no. 13, in column 5, or in Table II A, application no. 13, column 8, or in Table II B, application no. 13, column 8, or a homolog or a fragment thereof;
(b) a nucleic acid molecule shown in Table I, application no. 13, column 5 or 8, preferably shown in Table I A, application no. 13, in column 5, or in Table I A, application no. 13, column 8, or in Table I B, application no. 13, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 13, preferably shown in Table II A, application no. 13, in column 5, or in Table II A, application no. 13, column 8, or in Table II B, application no. 13, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 13, preferably shown in Table I A, application no. 13, in column 5, or in Table I A, application no. 13, column 8, or in Table I B, application no. 13, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 13;
(i) a nucleic acid molecule encoding a polypeptide having the activity represented by a protein as depicted in column 5 of Table II, application no. 13;
(j) a nucleic acid molecule comprising a polynucleotide which is obtained by amplifying a cDNA library or a genomic library using the primers in Table III, application no. 13, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto;
   whereby, preferably, the nucleic acid molecule according to 1 (a), (b), (c), (d), (e), (f), (g), (h), (i), (j) or (k) distinguishes over the sequence depicted in Table I A and/or I B, application no. 13, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 13, column 5 or 8, or the coding region thereof, respectively. In another embodiment, the nucleic acid molecule of the present invention, preferably the coding region thereof, is 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 13, column 5 or 8, or the coding region thereof, respectively. In a further embodiment the nucleic acid molecule does not encode the polypeptide sequence shown in Table II A and/or II B, application no. 13, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 13, column 5 or 8 and does not encode a protein of the sequence shown in Table II A and/or II B, application no. 13, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 13, column 5 and 8, or the coding region thereof. In a further embodiment, the protein of the present invention is at least 30%,40%, 50%, 60%, 70%, 80%, 90%, 95% identical to protein sequence depicted in Table II A and/or II B, application no. 13, column 5 or 8 but less than 100%, preferably less than 99.999%, 99.99% or 99.9%, more preferably less than 99%, 985, 97%, 96% or 95% identical to the sequence shown in Table II A and/or II B, application no. 13, column 5 or 8.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 13.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 13, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 13.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 13, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 13, in the same line as of the respective nucleic acid molecule according to 1) “plastidic” is depicted, or encoding a mitochondrial transit peptide, especially in case in column 6 of Table I, application no. 13, in the same line as of the respective nucleic acid molecule according to 1) “mitochondric” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 13, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 13, in the same line as of the respective nucleic acid molecule according to 1) “plastidic” is depicted, or encoding a mitochondrial transit peptide, especially in case in column 6 of Table I, application no. 13, in the same line as of the respective nucleic acid molecule according to 1) “mitochondric” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 13, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 13, in the same line as of the respective nucleic acid molecule according to 1) “plastidic” is depicted, or encoding a mitochondrial transit peptide, especially in case in column 6 of Table I, application no. 13, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 13.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 13, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 13.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 13, in the same line as of the respective nucleic acid molecule according to 1) “plastidic” is depicted, or encoding a mitochondrial transit peptide, especially in case in column 6 of Table I, application no. 13, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 13, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 13, in the same line as of the respective nucleic acid molecule according to 1) “plastidic” is depicted, or encoding a mitochondrial transit peptide, especially in case in column 6 of Table I, application no. 13, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 13 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 13, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 13 and confers a generation or an increase of the respective fine chemical in a non-human organism or a part thereof as compared to a corresponding wild-type non-human organism or part thereof.

[0181.1.7.13] to [0209.1.7.13] for the disclosure of these paragraphs see [0181.1.7.7] to [0209.1.7.7] above

The genes of the invention, coding in one embodiment for an activity selected from the group consisting of acetyl CoA carboxylase, acetyltransferase, acyl transferase, alcohol dehydrogenase, AX653549-protein, b1003-protein, b2032-protein, b2099-protein, b2121-protein, b2474-protein, b3346-protein, b3817-protein, coproporphyrinogen oxidase, F-box protein, flavodoxin, gluconate transport system permease 3, glutaredoxin, glycerol-3-phosphate dehydrogenase, glycine cleavage system T aminomethyltransferase, halotolerance protein, heat shock transcription factor, isopropylmalate isomerase large subunit, ketol-acid reductoisomerase, lipoprotein precursor, MADS-box transcription factor, melibiose carrier protein, monothiol glutaredoxin, monthiol glutaredoxin, N-acetylglucosaminylphosphatidylinositoldeacetylase, nitrate/nitrite transport protein, protein kinase, protein phosphatase, purine-nucleoside phosphorylase, ribosephosphate isomerase, constitutive, serine/threonine-protein phosphatase, sterol O-acyltransferase, superoxide dismutase, thioredoxin, transcription factor, transcriptional regulator, YCL026C-A-protein, YER184C-protein, ygl096w-protein, ymr324c-protein, and YOR044W-protein are also called “FCRP genes”.

[0211.1.7.13] to [0225.1.7.13] for the disclosure of these paragraphs see [0211.1.7.7] to [0225.1.7.7] above

In addition to the sequence mentioned in Table I, application no. 13, column 5 or 8, preferably the coding region thereof, or its homologs or fragments, it may be advantageous additionally to express and/or mutate further genes in the non-human organisms. Especially advantageously, additionally at least one further gene of the biosynthetic pathway of the fine chemical is expressed in the non-human organisms such as plants or microorganisms. It is also possible that the regulation of the natural genes has been modified advantageously so that the gene and/or its gene product is no longer subject to the regulatory mechanisms which exist in the non-human organisms. This leads to an increased synthesis of the respective fine chemical since, for example, feedback regulations no longer exist to the same extent or not at all. In addition it might be advantageously to combine the nucleic acids sequences of the invention containing the sequences shown in the respective line in Table I, application no. 13, column 5 or 8, preferably the coding region thereof, or homologs or fragments thereof, with genes which generally support or enhance the growth or yield of the target non-human organisms, for example genes which lead to faster growth rate of non human organism like microorganisms or plants or genes which produce stress-, pathogen-, or herbicide-resistant plants.

Further advantageous nucleic acid sequences which can be expressed in combination with the sequences used in the process and/or the abovementioned biosynthesis genes are the sequences encoding further genes of the carotenoids biosynthetic pathway, such as phytoene synthase (Psy), which is an important control point for the regulation of the flux (Fraser et al., 2002), phytoene desaturase (Pds), z-carotene desaturase, above mentioned enzymes (s. introduction of the application), e.g. hydroxylases such as beta-carotene hydroxylase (U.S. Pat. No. 6,214,575), ketolases, or cyclases such as the beta-cyclase (U.S. Pat. No. 6,232,530) or oxygenases such as the beta-C4-oxygenase described in U.S. Pat. No. 6,218,599 or homologs thereof, astaxanthin synthase (U.S. Pat. No. 6,365,386), or other genes as described in U.S. Pat. No. 6,150,130.

Other sequences which can be expressed in combination with the sequences used in the process and/or the abovementioned biosynthesis genes are the sequences encoding further genes of the carotenoid biosynthesis pathways, e.g. carotene 7,8-desaturase (EC 1.14.99.30), lycopene β-cyclase (EC:1.14.-.-), β-carotene hydroxylase, zeaxanthin epoxidase (EC:1.14.13.90),

If not an increase in violaxanthin contend alone is desired, but the increase in this importand protective compound is intended to enhance plant health, overexpression of then genes of the xanthophyll cycle might be of interest, e.g.: zeaxanthin epoxidase (E.C. 1.14.13.90), antheraxanthin epoxidase (E.C. 1.14.13.90), violaxanthin deepoxidase (E.C. 1.10.99.3).

Other sequences which can be expressed in combination with the sequences used in the process and/or the abovementioned biosynthesis genes are the sequences encoding further genes of the carotenoid biosynthesis pathways, e.g. 9-cis-epoxycarotenoid dioxygenase (NCED, EC: 1.13.11.51), catalysing the oxidative cleavage of 9-cis-violoxanthin or 9-cis-neoxanthin (synthesized from all-trans-violoxanthin) to produce 9-cis-xanthoxin.

In addition, for increased ABA content, genes from the carotenoid biosynthesis pathway could be overexpressed, e.g.: carotene 7,8-desaturase (EC 1.14.99.30), lycopene β-cyclase (EC 1.14.-.-), β-carotene hydroxylase, zeaxanthin epoxidase (EC 1.14.13.90), neoxanthin synthase (EC 5.3.99.9)

These genes can lead to an increased synthesis of the essential carotenoids, in particular xanthophylls.

[0228.1.7.13] to [0239.1.7.13] for the disclosure of these paragraphs see [0228.1.7.7] to [0239.1.7.7] above

In an embodiment of the present invention the nucleic acid construct of the present invention comprises a nucleic acid molecule as depicted in the respective line in Table I, application no. 13, column 5 or 8, preferably the coding region thereof, a fragment or a homolog thereof, and a plant promoter, such as the Big 35S promoter, the DC3 promoter, the PC Ubi promoter, the LegB4-promoter, the Super promoter, the USP promoter or the like. In another embodiment the nucleic acid construct of the present invention comprises a nucleic acid molecule as depicted in the respective line in Table I, application no. 13, column 5 or 8, preferably the coding region thereof, a fragment or a homolog thereof, and a plant promoter such as, for example, the USP, the LegB4-, the DC3 promoter or the ubiquitin promoter from parsley or other herein mentioned promoters. In a further embodiment the nucleic acid construct of the present invention comprises a nucleic acid molecule as depicted in the respective line in Table I, application no. 13, column 5 or 8, preferably the coding region thereof, a fragment or a homolog thereof, and a plant promoter, such as the Big 35S promoter, the PC Ubi promoter, the Super promoter, the USP promoter or the like. In addition different terminators may advantageously be used in these nucleic acid constructs; examples for transcriptional termination are polyadenylation signals.

[0241.1.7.13] to [0245.1.7.13] for the disclosure of these paragraphs see [0241.1.7.7] to [0245.1.7.7] above

Other preferred sequences for use in operable linkage in gene expression constructs are targeting sequences, which are required for targeting the gene product into specific cell compartments (for a review, see Kermode, Crit. Rev. Plant Sci. 15 (4), 285 (1996) and references cited therein), for example into the vacuole, the nucleus, all types of plastids, such as amyloplasts, chloroplasts, chromoplasts, the extracellular space, the mitochondria, the endoplasmic reticulum, elaioplasts, peroxisomes, glycosomes, and other compartments of cells or extracellular; preferred are sequences, which are involved in targeting to plastids as mentioned above. Sequences, which must be mentioned in this context are, in particular, the signal-peptide- or transit-peptide-encoding sequences which are known per se. For example, plastid-transit-peptide-encoding sequences enable the targeting of the expression product into the plastids of a plant cell. Targeting sequences are also known for eukaryotic and to a lower extent for prokaryotic organisms and can advantageously be operable linked with the nucleic acid molecule of the present invention as shown in Table I, application no. 13, columns 5 and 8, preferably the coding region thereof, or homologs or fragments thereof, and described herein to achieve an expression in one of said compartments or extracellular.

[0247.1.7.13] to [0249.1.7.13] for the disclosure of these paragraphs see [0247.1.7.7] to [0249.1.7.7] above

Preferred promoters are in particular those which bring about gene expression in tissues and organs, such as leaves, roots, seeds, pollen or xylem. Examples of tissue preferred or organ preferred promoters include, but are not limited to fruit-preferred, ovule-preferred, male tissue-preferred, seed-preferred, pollen-preferred integument-preferred, tuber-preferred, stalk-preferred, pericarp-preferred, leaf-preferred, stigma-preferred, pollen-preferred, anther-preferred, petal-preferred, sepal-preferred, pedicel-preferred, silique-preferred, stem-preferred, root-preferred promoters, and the like. Especially those promoters are preferred which bring about gene expression in tissues and organs in which the biosynthesis of xanthophylls and/or abscisic acid resp., in particular, of abscisic acid, cryptoxanthin, violaxanthin, or zeaxanthin takes place, like in seed cells, such as endosperm cells and cells of the developing embryo. Seed promoters are preferentially expressed during seed development and/or germination. For example, seed preferred promoters can be embryo-preferred, endosperm preferred and seed coat-preferred (see Thompson et al., BioEssays 10, 108 (1989)). Examples of seed preferred promoters include, but are not limited to, cellulose synthase (celA), Cim1, gammazein, globulin-1, maize 19 kD zein (cZ19B1), and the like. Other suitable promoters are the oilseed rape napin gene promoter (U.S. Pat. No. 5,608,152), the Vicia faba USP promoter (Baeumlein et al., Mol Gen Genet, 225 (3), 459 (1991)), the Arabidopsis oleosin promoter (WO 98/45461), the Phaseolus vulgaris phaseolin promoter (U.S. Pat. No. 5,504,200), the Brassica Bce4 promoter (WO 91/13980), the bean arc5 promoter, the carrot DcG3 promoter, or the Legumin B4 promoter (LeB4) (Baeumlein et al., Plant Journal, 2 (2), 233 (1992)), and promoters which bring about the seed-specific expression in monocotyledonous plants such as maize, barley, wheat, rye, rice and the like. Advantageous seed-specific promoters are the sucrose binding protein promoter (WO 00/26388), the phaseolin promoter and the napin promoter. Suitable promoters which must be considered are the barley Ipt2 or Ipt1 gene promoter (WO 95/15389 and WO 95/23230), and the promoters described in WO 99/16890 (promoters from the barley hordein gene, the rice glutelin gene, the rice oryzin gene, the rice prolamin gene, the wheat gliadin gene, the wheat glutelin gene, the maize zein gene, the oat glutelin gene, the sorghum kasirin gene and the rye secalin gene). Further suitable promoters are Amy32b, Amy 6-6 and Aleurain (U.S. Pat. No. 5,677,474), Bce4 (oilseed rape) (U.S. Pat. No. 5,530,149), glycinin (soya) (EP 571 741], phosphoenolpyruvate carboxylase (soya) (JP 06/62870), ADR12-2 (soya) (WO 98/08962), isocitrate lyase (oilseed rape) (U.S. Pat. No. 5,689,040]) or α-amylase (barley) (EP 781 8499. Other promoters which are available for the expression of genes in plants are leaf-specific promoters such as those described in DE-A 19 644 478 or light-regulated promoters such as, for example, the pea petE promoter.

[0251.1.7.13] to [0266.1.7.13] for the disclosure of these paragraphs see [0251.1.7.7] to [0266.1.7.7] above

An embodiment of the invention relates to a vector where the nucleic acid molecule according to the invention is linked operably to regulatory sequences which permit the expression in a prokaryotic or eukaryotic host. A further embodiment of the invention relates to a vector in which a nucleic acid sequence encoding one of the polypeptides shown in the respective line in Table II, application no. 13, columns 5 or 8, or homologs or fragments thereof, is functionally linked to a targeting sequence such as a plastidial or mitochondrial targeting sequence.

A further embodiment of the invention relates to a vector in which a nucleic acid sequence encoding one of the polypeptides shown in the respective line in Table II, application no. 13, columns 5 or 8, or homologs or fragments thereof, is functionally linked to (a) regulatory sequence which permit the expression in an organelle, such as plastids or mitochondria, preferably plastids.

A further embodiment of the invention relates to a vector which comprises a nucleic acid sequence encoding one of the polypeptides shown in the respective line in Table II, application no. 13, columns 5 or 8, or homologs or fragments thereof, but no additional targeting sequence.

[0268.1.7.13] to [0273.1.7.13] for the disclosure of these paragraphs see [0268.1.7.7] to [0273.1.7.7] above

A further object of the invention relates to the use of a nucleic acid construct, e.g. an expression cassette, comprising one or more nucleic acid molecules encoding one or more polypeptides as depicted in the respective line(s) in Table II, application no. 13, columns 5 or 8, or homologs or fragments thereof, or comprising one or more nucleic acid molecules as depicted in the respective line(s) in Table I, application no. 13, columns 5 or 8, preferably the coding region thereof, or homologs or fragments thereof, or comprising a nucleic acid molecule hybridizing therewith for the transformation of a microorganism or a plant cell, plant tissues or a part of a plant

In doing so, depending on the choice of promoter, the nucleic acid molecules shown in the respective line in Table I, application no. 13, column 5 or 8, preferably the coding region thereof, or homologs or fragments thereof, or the nucleic acid molecules encoding a polypeptide as depicted in the respective line in Table II, application no. 13, column 5 or 8, or homologs or fragments thereof, can be expressed in the whole plant or specifically e.g. in the leaves, in the seeds, the nodules, in roots, in the stem or other parts of the plant. Those transgenic plants overproducing sequences, e.g. as depicted in the respective line in Table I, application no. 13, column 5 or 8, preferably the coding region thereof, or homologs or fragments thereof, the plant cell, plant tissues, plants, the reproductive material thereof, or parts thereof are a further object of the present invention.

Within the framework of the present invention, the production of the abscisic acid, cryptoxanthin, violaxanthin, or zeaxanthin is due to the generation or over-expression of one or more polypeptides as depicted in the respective line(s) in Table II, application no. 13, column 5 or 8, or homologs or fragments thereof, or encoded by the corresponding nucleic acid molecules as depicted in the respective line(s) in Table I, application no. 13, column 5 or 8, preferably the coding region thereof, or homologs or fragments thereof, in the non-human organism according to the invention, advantageously in the transgenic microorganism or plant according to the invention, at least for the duration of at least one plant generation.

A constitutive expression of the polypeptide as depicted in the respective line in Table II, application no. 13, column 5 or 8, or homologs or fragments thereof, or encoded by the respective nucleic acid molecule as depicted in the respective line in Table I, application no. 13, column 5 or 8, preferably the coding region thereof, or homologs or fragments thereof, may be advantageous. On the other hand, an inducible expression may also appear desirable. Expression of the polypeptide sequences of the invention can be either directed to the cytosol or to the organelles, such as plastids or mitochondria, preferably the plastids of the host cells, preferably the plant cells, or non-targeted.

for the disclosure of this paragraph see [0278.1.7.7] above

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 13, column 5 or 8, preferably shown in Table II A, application no. 13, column 5, or in Table II A, application no. 13, column 8, or in Table II B, application no. 13, column 8, or a homolog or a fragment thereof;
   (b) a nucleic acid molecule shown in Table I, application no. 13, column 5 or 8, preferably shown in Table I A, application no. 13, column 5, or in Table I A, application no. 13, column 8, or in Table I B, application no. 13, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 13, preferably shown in Table II A, application no. 13, column 5, or in Table II A, application no. 13, column 8, or in Table II B, application no. 13, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 13, preferably shown in Table I A, application no. 13, column 5, or in Table I A, application no. 13, column 8, or in Table I B, application no. 13, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 13;
   (i) a nucleic acid molecule encoding a polypeptide having the activity represented by a respective protein as depicted in column 5 of Table II, application no. 13;
   (j) a nucleic acid molecule comprising a polynucleotide which is obtained by amplifying a cDNA library or a genomic library using the primers in Table III, application no. 13, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising an expression cassette comprising
[0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 13, column 5 or 8, preferably shown in Table II A, application no. 13, column 5, or in Table II A, application no. 13, column 8, or in Table II B, application no. 13, column 8, or a homolog or a fragment thereof;
   (b) a nucleic acid molecule shown in Table I, application no. 13, column 5 or 8, preferably shown in Table I A, application no. 13, column 5, or in Table I A, application no. 13, column 8, or in Table I B, application no. 13, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 13, preferably shown in Table II A, application no. 13, column 5, or in Table II A, application no. 13, column 8, or in Table II B, application no. 13, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 13, preferably shown in Table I A, application no. 13, column 5, or in Table I A, application no. 13, column 8, or in Table I B, application no. 13, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 13;
   (i) a nucleic acid molecule encoding a polypeptide having the activity represented by a respective protein as depicted in column 5 of Table II, application no. 13;
   (j) a nucleic acid molecule comprising a polynucleotide which is obtained by amplifying a cDNA library or a genomic library using the primers in Table III, application no. 13, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising or being transformed by a vector comprising
[1) a nucleic acid molecule selected from the group consisting of
   (a) a nucleic acid molecule encoding a polypeptide shown in Table II, application no. 13, column 5 or 8, preferably shown in Table II A, application no. 13, column 5, or in Table II A, application no. 13, column 8, or in Table II B, application no. 13, column 8, or a homolog or a fragment thereof;
   (b) a nucleic acid molecule shown in Table I, application no. 13, column 5 or 8, preferably shown in Table I A, application no. 13, column 5, or in Table I A, application no. 13, column 8, or in Table I B, application no. 13, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 13, preferably shown in Table II A, application no. 13, column 5, or in Table II A, application no. 13, column 8, or in Table II B, application no. 13, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 13, preferably shown in Table I A, application no. 13, column 5, or in Table I A, application no. 13, column 8, or in Table I B, application no. 13, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 13;
   (i) a nucleic acid molecule encoding a polypeptide having the activity represented by a respective protein as depicted in column 5 of Table II, application no. 13;
   (j) a nucleic acid molecule comprising a polynucleotide which is obtained by amplifying a cDNA library or a genomic library using the primers in Table III, application no. 13, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 13.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 13 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 13, in the same line as of the respective nucleic acid molecule according to 1) “plastidic” is depicted, or encoding a mitochondrial transit peptide, especially in case in column 6 of Table I, application no. 13, in the same line as of the respective nucleic acid molecule according to 1) “mitochondric” is depicted.

In another embodiment of said transgenic non-human organism the expression cassette does not comprise 2) a nucleic acid molecule encoding a transit peptide, especially in case in column 6 of Table I, application no. 13, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 13.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 13, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 13, in the same line as of the respective nucleic acid molecule according to 1) “plastidic” is depicted, or encoding a mitochondrial transit peptide, especially in case in column 6 of Table I, application no. 13, in the same line as of the respective nucleic acid molecule according to 1) “mitochondric” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 13, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 13, in the same line as of the respective nucleic acid molecule according to 1) “plastidic” is depicted, or encoding a mitochondrial transit peptide, especially in case in column 6 of Table I, application no. 13, in the same line as of the respective nucleic acid molecule according to 1) “mitochondric” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 13, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 13, in the same line as of the respective nucleic acid molecule according to 1) “plastidic” is depicted, or encoding a mitochondrial transit peptide, especially in case in column 6 of Table I, application no. 13, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 13.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi,

Super and USP; a nucleic acid molecule encoding a transit peptide, especially in case in column 6 of Table I, application no. 13, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 13.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 13, in the same line as of the respective nucleic acid molecule according to 1) “plastidic” is depicted, or encoding a mitochondrial transit peptide, especially in case in column 6 of Table I, application no. 13, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 13, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 13, in the same line as of the respective nucleic acid molecule according to 1) “plastidic” is depicted, or encoding a mitochondrial transit peptide, especially in case in column 6 of Table I, application no. 13, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 13 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 13, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 13 (in the same line) and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 13, column 5 or 8, preferably shown in Table II A, application no. 13, column 5, or in Table II A, application no. 13, column 8, or in Table II B, application no. 13, column 8, or a homolog or a fragment thereof;
   (b) a nucleic acid molecule shown in Table I, application no. 13, column 5 or 8, preferably shown in Table I A, application no. 13, column 5, or in Table I A, application no. 13, column 8, or in Table I B, application no. 13, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 13, preferably shown in Table II A, application no. 13, column 5, or in Table II A, application no. 13, column 8, or in Table II B, application no. 13, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 13, preferably shown in Table I A, application no. 13, column 5, or in Table I A, application no. 13, column 8, or in Table I B, application no. 13, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 13;
   (i) a nucleic acid molecule encoding a polypeptide having the activity represented by a respective protein as depicted in column 5 of Table II, application no. 13;
   (j) a nucleic acid molecule comprising a polynucleotide which is obtained by amplifying a cDNA library or a genomic library using the primers in Table III, application no. 13, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising an expression cassette comprising
[0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 13, column 5 or 8, preferably shown in Table II A, application no. 13, column 5, or in Table II A, application no. 13, column 8, or in Table II B, application no. 13, column 8, or a homolog or a fragment thereof;
   (b) a nucleic acid molecule shown in Table I, application no. 13, column 5 or 8, preferably shown in Table I A, application no. 13, column 5, or in Table I A, application no. 13, column 8, or in Table I B, application no. 13, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 13, preferably shown in Table II A, application no. 13, column 5, or in Table II A, application no. 13, column 8, or in Table II B, application no. 13, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 13, preferably shown in Table I A, application no. 13, column 5, or in Table I A, application no. 13, column 8, or in Table I B, application no. 13, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 13;
   (i) a nucleic acid molecule encoding a polypeptide having the activity represented by a respective protein as depicted in column 5 of Table II, application no. 13;
   (j) a nucleic acid molecule comprising a polynucleotide which is obtained by amplifying a cDNA library or a genomic library using the primers in Table III, application no. 13, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      and, if desired
2) nucleic acid molecule encoding a transit peptide, preferably encoding a plastidal transit peptide or a mitochondrial transit peptide;
   which are operable linked;]
   or comprising or being transformed by a vector comprising
[1) a nucleic acid molecule selected from the group consisting of
   (a) a nucleic acid molecule encoding a polypeptide shown in Table II, application no. 13, column 5 or 8, preferably shown in Table II A, application no. 13, column 5, or in Table II A, application no. 13, column 8, or in Table II B, application no. 13, column 8, or a homolog or a fragment thereof;
   (b) a nucleic acid molecule shown in Table I, application no. 13, column 5 or 8, preferably shown in Table I A, application no. 13, column 5, or in Table I A, application no. 13, column 8, or in Table I B, application no. 13, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 13, preferably shown in Table II A, application no. 13, column 5, or in Table II A, application no. 13, column 8, or in Table II B, application no. 13, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 13, preferably shown in Table I A, application no. 13, column 5, or in Table I A, application no. 13, column 8, or in Table I B, application no. 13, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 13;
   (i) a nucleic acid molecule encoding a polypeptide having the activity represented by a respective protein as depicted in column 5 of Table II, application no. 13;
   (j) a nucleic acid molecule comprising a polynucleotide which is obtained by amplifying a cDNA library or a genomic library using the primers in Table III, application no. 13, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      whereby, preferably, the nucleic acid molecule according to 1 (a), (b), (c), (d), (e), (f), (g), (h), (i), (j) or (k) distinguishes over the sequence depicted in Table I A and/or I B, application no. 13, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 13, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 13, column 5 or 8, or the coding region thereof, respectively. In a further embodiment the nucleic acid molecule does not encode the polypeptide sequence shown in Table II A and/or II B, application no. 13, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 13, column 5 or 8 and does not encode a protein of the sequence shown in Table II A and/or II B, application no. 13, column 5 or 8. Accordingly, in one embodiment, the protein encoded by a sequence of a nucleic acid according to 1 (a), (b), (c), (d), (e), (f), (g), (h), (i), (j) or (k) does not consist of the sequence shown in Table I A and/or I B, application no. 13, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 13, column 5 or 8 but less than 100%, preferably less than 99.999%, 99.99%, 99.9%, 99%, 98%, 97%, 96% or 95% identical to the sequence shown in Table II A and/or II B, application no. 13, column 5 or 8;
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 13.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 13 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 13, in the same line as of the respective nucleic acid molecule according to 1) “plastidic” is depicted, or encoding a mitochondrial transit peptide, especially in case in column 6 of Table I, application no. 13, in the same line as of the respective nucleic acid molecule according to 1) “mitochondric” is depicted.

In another embodiment thereof the expression cassette does not comprise 2) a nucleic acid molecule encoding a transit peptide, especially in case in column 6 of Table I, application no. 13, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 13.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the same line in column 5 of Table II, application no. 13, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 13, in the same line as of the respective nucleic acid molecule according to 1) “plastidic” is depicted, or encoding a mitochondrial transit peptide, especially in case in column 6 of Table I, application no. 13, in the same line as of the respective nucleic acid molecule according to 1) “mitochondric” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 13, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 13, in the same line as of the respective nucleic acid molecule according to 1) “plastidic” is depicted, or encoding a mitochondrial transit peptide, especially in case in column 6 of Table I, application no. 13, in the same line as of the respective nucleic acid molecule according to 1) “mitochondric” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 13, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 13, in the same line as of the respective nucleic acid molecule according to 1) “plastidic” is depicted, or encoding a mitochondrial transit peptide, especially in case in column 6 of Table I, application no. 13, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 13.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 13, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 13.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 13, in the same line as of the respective nucleic acid molecule according to 1) “plastidic” is depicted, or encoding a mitochondrial transit peptide, especially in case in column 6 of Table I, application no. 13, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 13, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 13, in the same line as of the respective nucleic acid molecule according to 1) “plastidic” is depicted, or encoding a mitochondrial transit peptide, especially in case in column 6 of Table I, application no. 13, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 13 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 13, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 13 and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

[0291.1.7.13] to [0299.1.7.13] for the disclosure of these paragraphs see [0291.1.7.7] to [0299.1.7.7] above

The term “biological active portion” or “biological activity” means a polypeptide as depicted in the respective line in Table II, application no. 13, column 3 or a portion of said polypeptide which still has at least 10% or 20%, preferably 30%, 40%, 50% or 60%, especially preferably 70%, 75%, 80%, 90% or 95% of the enzymatic or biological activity of the natural or starting enzyme or protein.

[0301.1.7.13] to [0304.1.7.13] for the disclosure of these paragraphs see [0301.1.7.7] to [0304.1.7.7] above

Synthetic oligonucleotide primers for the amplification, e.g. as shown in the respective line in Table III, application no. 13, column 8, by means of polymerase chain reaction can be generated on the basis of a sequence shown herein, for example the sequence shown in the respective line in Table I, application no. 13, columns 5 or 8, or the sequences derived from Table II, application no. 13, columns 5 or 8, respectively.

Moreover, it is possible to identify a conserved region of a protein from various organism by carrying out protein sequence alignments with the polypeptide encoded by the nucleic acid molecules of the present invention, in particular with the sequences encoded by the nucleic acid molecule shown in the respective line in column 5 or 8 of Table I, application no. 13, from which conserved regions, and in turn, degenerate primers can be derived. Conserved regions are those, which show a very little variation in the amino acid in one particular position of several homologs from different origin. The consensus sequence and polypeptide motifs shown in the respective line in column 8 of Table IV, application no. 13, are derived from such alignments of homologous proteins.

In an embodiment of the present invention, in the method of the present invention the activity of a polypeptide comprising or consisting of a consensus sequence or at least one polypeptide motif shown in the respective line in Table IV, application no. 13, column 8, is increased. In another embodiment thereof, the method of the present invention relates to a polypeptide comprising or consisting of a consensus sequence shown in the respective line in Table IV, application no. 13, column 8 or comprising at least one polypeptide motif shown in the respective line in Table IV, application no. 13, column 8, whereby not more than 20, 15, 10, 9, 8, 7, 6, 5 4, 3, 2 or 1, or 0 of the amino acid positions indicated can be replaced by any amino acid. In another embodiment thereof, the method of the present invention relates to a polypeptide comprising or consisting of a consensus sequence shown in the respective line in Table IV, application no. 13, column 8 or comprising at least one polypeptide motif shown in the respective line in Table IV, application no. 13, column 8, whereby not more than 15%, 10%, 5%, 4%, 3%, 2% or 1%, or 0% of the amino acid positions defined as distinct specific amino acids are/is replaced by another amino acid. In another embodiment thereof, the method of the present invention relates to a polypeptide comprising or consisting of a consensus sequence shown in the respective line in Table IV, application no. 13, column 8 or comprising at least one polypeptide motif shown in the respective line in Table IV, application no. 13, column 8, whereby not more than 20, 15, 10, 9, 8, 7, 6, 5, 4, 3, 2 or 1, or 0 amino acids are inserted into a consensus sequence or polypeptide motif.

In an embodiment of the present invention, in the method of the present invention the activity of a polypeptide comprising or consisting of a consensus sequence or at least one polypeptide motif shown in the respective line in Table IV, application no. 13, column 8, is increased, whereby said polypeptide distinguishes over the sequence depicted in Table II, application no. 13, columns 5 or 8 by one or more amino acids.

In one embodiment, said polypeptide of the invention distinguishes over the sequence shown in the respective line in Table II, application no. 13, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II B, application no. 13, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in Table II, application no. 13, columns 5 or 8.

[0309.1.7.13] to for the disclosure of these paragraphs see [0309.1.7.7] to [0321.1.7.7] above

Polypeptides having above-mentioned activity, i.e. conferring the production or the increased production of the fine chemical abscisic acid, cryptoxanthin, violaxanthin, or zeaxanthin as compared to a corresponding, e.g. non-transformed, wild type non-human organism, like a microorganism or a plant cell, plant or part thereof, can be encoded by other DNA sequences which hybridize to the sequences shown in the respective line in Table I, application no. 13, columns 5 and 8, preferably the coding region thereof, at least under relaxed hybridization conditions and which encode the expression of polypeptides conferring the production or the increased production of the respective fine chemical abscisic acid, cryptoxanthin, violaxanthin, or zeaxanthin as compared to a corresponding, e.g. non-transformed, wild type non-human organism, like a microorganism or a plant cell, plant or part thereof.

[0323.1.7.13] to for the disclosure of these paragraphs see [0323.1.7.7] to [0329.1.7.7] above

Further, the nucleic acid molecule of the invention comprises a nucleic acid molecule, which is a complement of one of the nucleotide sequences of above-mentioned nucleic acid molecules or a portion thereof. A nucleic acid molecule or its sequence which is complementary to one of the nucleotide molecules or sequences shown in the respective line in Table I, application no. 13, columns 5 or 8, preferably the coding region thereof, homologs or fragments thereof, is one which is sufficiently complementary to one of the nucleotide molecules or sequences shown in the respective line in Table I, application no. 13, columns 5 or 8, preferably the coding region thereof, homologs or fragments thereof, such that it can hybridize to one of the nucleotide sequences shown in Table I, application no. 13, columns 5 or 8, preferably the coding region thereof, homologs or fragments thereof, thereby forming a stable duplex.

Preferably, the hybridization is performed under stringent hybridization conditions. However, a complement of one of the herein disclosed sequences is preferably a sequence complement thereto according to the base pairing of nucleic acid molecules well known to the skilled person. For example, the bases A and G undergo base pairing with the bases T and U or C, resp. and visa versa. Modifications of the bases can influence the base-pairing partner.

The nucleic acid molecule of the invention comprises a nucleotide sequence which is at least about 30%, 35%, 40%, 45%, 50%, 55%, 60%, 65%, 70%, 75%, 80%, 90%, 95%, 96%, 97%, 98%, 99%, 99.5% or more homologous to a nucleotide sequence shown in Table I, application no. 13, columns 5 or 8, preferably the coding region thereof, homologs or fragments thereof, and preferably has above-mentioned activity, in particular conferring the production or the increased production of the fine chemical abscisic acid, cryptoxanthin, violaxanthin, or zeaxanthin, respectively, after increasing the activity or an activity of a gene as shown in the respective line in Table I or of a gene product, e.g. as shown in the respective line in Table II, application no. 13, column 5 or 8, by for example in one embodiment expression either in the cytosol or in an organelle such as a plastid or mitochondria or both, preferably in a plastid, or in another embodiment non-targeted or targeted.

The nucleic acid molecule of the invention comprises a nucleotide sequence or molecule which hybridizes, preferably hybridizes under stringent conditions as defined herein, to one of the nucleic acid molecule shown in the respective line in Table I, preferably Table IB, application no. 13, columns 5 or 8, preferably the coding region thereof, fragments or homologs thereof, and encodes a protein having above-mentioned activity, e.g. conferring the production or the increased production of the respective fine chemical abscisic acid, cryptoxanthin, violaxanthin, or zeaxanthin as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted, and optionally, the activity thereof is selected from the group consisting of acetyl CoA carboxylase, acetyltransferase, acyl transferase, alcohol dehydrogenase, AX653549-protein, b1003-protein, b2032-protein, b2099-protein, b2121-protein, b2474-protein, b3346-protein, b3817-protein, coproporphyrinogen oxidase, F-box protein, flavodoxin, gluconate transport system permease 3, glutaredoxin, glycerol-3-phosphate dehydrogenase, glycine cleavage system T aminomethyltransferase, halotolerance protein, heat shock transcription factor, isopropylmalate isomerase large subunit, ketol-acid reductoisomerase, lipoprotein precursor, MADS-box transcription factor, melibiose carrier protein, monothiol glutaredoxin, monthiol glutaredoxin, N-acetylglucosaminylphosphatidylinositoldeacetylase, nitrate/nitrite transport protein, protein kinase, protein phosphatase, purine-nucleoside phosphorylase, ribosephosphate isomerase, constitutive, serine/threonine-protein phosphatase, sterol O-acyltransferase, superoxide dismutase, thioredoxin, transcription factor, transcriptional regulator, YCL026C-A-protein, YER184C-protein, ygl096w-protein, ymr324c-protein, and YOR044W-protein, respectively.

Moreover, the nucleic acid molecule of the invention can comprise only a porganism tion of the coding region of one of the nucleic acid molecules depicted in Table I, application no. 13, columns 5 or 8, for example a fragment which can be used as a probe or primer or a fragment encoding a biologically active portion of the polypeptide of the present invention or of a polypeptide used in the process of the present invention, i.e. having above-mentioned activity, e.g. conferring the production or the increased production of the respective fine chemical abscisic acid, cryptoxanthin, violaxanthin, or zeaxanthin as compared to a corresponding, e.g. non-transformed, wild type non-human organism, like a microorganism or a plant cell, plant or part thereof, by for example in one embodiment expression either in the cytosol or in an organism elle such as a plastid or mitochondria or both, preferably in a plastid, or in another embodiment non-targeted or targeted. The nucleotide sequences determined from the cloning of the present protein-according-to-the-invention-encoding gene allows for the generation of probes and primers designed for use in identifying and/or cloning its homologues from other cell types and organisms. The probe/primer typically comprises a substantially purified oligonucleotide. The oligonucleotide typically comprises a region of nucleotide sequence that hybridizes under stringent conditions to at least about 12, 15 preferably about 20 or 25, more preferably about 40, 50 or 75 consecutive nucleotides of a sense strand of one of the sequences set forth, e.g., in the respective line in Table I, application no. 13, column 5 or 8, preferably the coding region thereof, or a homolog or a fragment thereof, an anti-sense sequence of one of the sequences, e.g., set forth in the respective line in Table I, application no. 13, columns 5 or 8, preferably the coding region thereof, or a homolog or a fragment thereof or naturally occurring mutants thereof. Primers based on a nucleic acid molecule of invention can be used in PCR reactions to clone homologs of the polypeptide of the invention or of the polypeptide used in the process of the invention, e.g. as the primers described in the examples of the present invention, e.g. as shown in the examples. A PCR with the primers shown in the respective line in Table III, column 8 will result in a fragment of the gene product as shown in Table II, application no. 13, column 5.

Primer sets are interchangeable. The person skilled in the art knows how to design and combine said primers to result in the desired product, e.g. in a full length clone or a partial sequence. Probes based on the sequences of the nucleic acid molecule of the invention or used in the process of the present invention can be used to detect transcripts or genomic sequences encoding the same or homologous proteins. The probe can further comprise a label group attached thereto, e.g. the label group can be a radioisotope, a fluorescent compound, an enzyme, or an enzyme co-factor. Such probes can be used as a part of a genomic marker test kit for identifying cells which express a polypeptide of the invention or used in the process of the present invention, such as by measuring a level of an encoding nucleic acid molecule in a sample of cells, e.g. detecting mRNA levels or determining, whether a genomic gene comprising the sequence of the polynucleotide of the invention or used in the processes of the present invention has been mutated or deleted.

As the expression of a respective nucleic acids of the invention is related to the synthesis of the respective fine chemical abscisic acid, cryptoxanthin, violaxanthin, or zeaxanthin its function as a probe extends to the detection of microorganisms, plant tissues, plants, plant variets, plant ecotypes or plant genera with varying capability or potential for synthesis of the respective fine chemical abscisic acid, cryptoxanthin, violaxanthin, or zeaxanthin. Therefore in one embodiment the present invention relates to a method for analyzing the capability or potential of a plant tissue, a plant, a plant variety or plant ecotype to produce the fine chemical abscisic acid, cryptoxanthin, violaxanthin, or zeaxanthin by using the nucleic acid of the invention or parts thereof as a probe to detect the amount of the nucleic acid of the invention in the non-human organism or a part thereof in comparison to another non-human organism.

The nucleic acid molecule of the invention encodes a polypeptide or portion thereof which includes an amino acid sequence which is sufficiently homologous to the amino acid sequence shown in respective line in Table II, application no. 13, columns 5 or 8 such that the protein or portion thereof maintains the ability to confer the production or the increased production of the respective fine chemical abscisic acid, cryptoxanthin, violaxanthin, or zeaxanthin as compared to a corresponding, e.g. non-transformed, wild type non-human organism, like a microorganism or a plant cell, plant or part thereof, in particular increasing the activity as mentioned above or as described in the examples in microorganisms or plants.

As used herein, the language “sufficiently homologous” refers to proteins or portions thereof which have amino acid sequences which include a minimum number of identical or equivalent amino acids (e.g. an amino acid which has a similar side chain as an amino acid in one of the sequences of the polypeptide of the present invention) to an amino acid sequence shown in the respective line in Table II, application no. 13, columns 5 or 8 such that the protein or portion thereof is able to confer the production or the increased production of the respective fine chemical abscisic acid, cryptoxanthin, violaxanthin, or zeaxanthin as compared to a corresponding, e.g. non-transformed, wild type non-human organism, like a microorganism or a plant cell, plant or part thereof. For example having the activity of a protein as shown in the respective line in Table II, application no. 13, column 3 and as described herein.

In an embodiment of the present invention, the nucleic acid molecule of the present invention comprises a nucleic acid molecule that encodes a portion of the protein of the present invention. The protein is at least about 30%, 35%, 40%, 45% 50%, 55%, 60%, 65%, 70%, 75%, 80%, 85%, 90%, 91%, 92%, 93%, 94%, 95%, 96%, 97%, 98%, 99%, 99.5% or more homologous to an entire amino acid sequence shown in the respective line of Table II, application no. 13, column 5 or 8 and has the above-mentioned activity, e.g. conferring the production or the increased production of the respective fine chemical abscisic acid, cryptoxanthin, violaxanthin, or zeaxanthin as compared to a corresponding, e.g. non-transformed, wild type non-human organism, like a microorganism or a plant cell, plant or part thereof by, for example in a embodiment expression either in the cytosol or in an organelle such as a plastid or mitochondria or both, preferably in a plastid, or in another embodiment by targeted or non-targeted expression.

for the disclosure of this paragraph see [0338.1.7.7] above

for the disclosure of this paragraph see [0339.1.7.7] above

The invention further relates to nucleic acid molecules or methods using said nucleic acid molecules that differ from one of the nucleotide sequences shown in the respective line in Table I, application no. 13, columns 5 or 8, preferably the coding region thereof, or fragments or homologs thereof, due to degeneracy of the genetic code but encode a polypeptide of the present invention, in particular a polypeptide having the above-mentioned activity, e.g. as those polypeptides depicted in the respective line in Table II, application no. 13, columns 5 or 8, fragments or homologs thereof. Advantageously, the nucleic acid molecule of the invention comprises, or in an other embodiment has, a nucleotide sequence encoding a protein comprising, or in an other embodiment having, an amino acid sequence shown in the respective line in Table II, application no. 13, columns 5 or 8, fragments or homologs thereof. In a further embodiment, the nucleic acid molecule of the invention encodes a full length protein which is substantially homologous to an amino acid sequence shown in the respective line in Table II, application no. 13, columns 5 or 8, or homologs thereof. However, in an embodiment, the nucleic acid molecule of the present invention does not consist of the sequence shown in the respective line in Table I, preferably Table IA, application no. 13, columns 5 or 8, preferably the coding region thereof, but the functional homologs thereof.

[0341.1.7.13] to [0343.1.7.13] for the disclosure of these paragraphs see [0341.1.7.7] to [0343.1.7.7] above

Accordingly, in another embodiment, a nucleic acid molecule of the invention is at least 15, 20, 25 or 30 nucleotides in length. Preferably, it hybridizes under stringent conditions to a nucleic acid molecule comprising a nucleotide sequence of the nucleic acid molecule of the present invention or used in the process of the present invention, e.g. comprising the sequence shown in the respective line in Table I, application no. 13, columns 5 or 8, preferably the coding region thereof. In another embodiment the nucleic acid molecule is preferably at least 20, 30, 50, 100, 250 or more nucleotides in length.

for the disclosure of this paragraph see [0345.1.7.7] above

Preferably, a nucleic acid molecule of the invention that hybridizes under stringent conditions to a sequence shown in the respective line in Table I, application no. 13, columns 5 or 8, preferably the coding region thereof, corresponds to a naturally-occurring nucleic acid molecule of the invention. As used herein, a “naturally-occurring” nucleic acid molecule refers to a RNA or DNA molecule having a nucleotide sequence that occurs in nature (e.g. encodes a natural protein). Preferably, the nucleic acid molecule encodes a natural protein having above-mentioned activity, e.g. conferring the production or the increased production of the respective fine chemical abscisic acid, cryptoxanthin, violaxanthin, or zeaxanthin as compared to a corresponding, e.g. non-transformed, wild type non-human organism, like a microorganism or a plant cell, plant or part thereof after increasing the expression or activity thereof or the activity of a protein of the invention or used in the process of the invention, in an embodiment for example expression either in the cytosol or in an organelle such as a plastid or mitochondria, preferably in plastids, or, in another embodiment by targeted or non-targeted expression.

for the disclosure of this paragraph see [0347.1.7.7] above

For example, nucleotide substitutions leading to amino acid substitutions at “non-essential” amino acid residues can be made in a sequence of the nucleic acid molecule of the invention or used in the process of the invention, e.g. shown in the respective line in Table I, application no. 13, columns 5 or 8, preferably the coding region thereof, or fragments or homologs thereof.

for the disclosure of this paragraph see [0349.1.7.7] above

for the disclosure of this paragraph see [0350.1.7.7] above

Accordingly, the invention relates to nucleic acid molecules encoding a polypeptide having above-mentioned activity, in a non-human organisms or parts thereof by for example expression either in the cytosol or in an organelle such as a plastid or mitochondria or both, preferably in plastids, that contain changes in amino acid residues that are not essential for said activity. Such polypeptides differ in amino acid sequence from a sequence depicted in the respective line in Table II, especially Table IIA, application no. 13, columns 5 or 8, but retain said activity described herein. The nucleic acid molecule can comprise a nucleotide sequence encoding a polypeptide, wherein the polypeptide comprises an amino acid sequence at least about 50% identical to an amino acid sequence shown in the respective line in Table II, application no. 13, columns 5 or 8, and is capable of conferring the production or the increased production of the respective fine chemical abscisic acid, cryptoxanthin, violaxanthin, or zeaxanthin as compared to a corresponding, e.g. non-transformed, wild type non-human organism, like a microorganism or a plant cell, plant or part thereof, after increasing its activity for example in an embodiment by expression either in the cytosol or in an organelle such as a plastid or mitochondria or both, preferably in plastids, or, in another embodiment by targeted or non-targeted expression. Preferably, the protein encoded by the nucleic acid molecule is at least about 60%, 70%, 80%, 90%, 95%, 96%, 97%, 98%, 99% or 99.5% identical to the sequence shown in the respective line in Table II, application no. 13, columns 5 or 8.

[0352.1.7.13] to [0357.1.7.13] for the disclosure of these paragraphs see [0352.1.7.7] to [0357.1.7.7] above

Functional equivalents of the nucleic acid molecules according to the present invention and/or used in the process according to the present invention represent an embodiment of homologs. Functional equivalents derived from the nucleic acid molecules as shown in the respective line in Table I, application no. 13, columns 5 or 8, preferably the coding region thereof, according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the nucleic acid molecules as shown in the respective line in Table I, application no. 13, columns 5 or 8, preferably the coding region thereof, according to the invention and encode polypeptides having essentially the same properties as the polypeptide as shown in the respective line in Table II, application no. 13, columns 5 or 8.

Functional equivalents derived from one of the polypeptides as shown in the respective line in Table II, application no. 13, columns 5 or 8 according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the polypeptides as shown in the respective line in Table II, application no. 13, columns 5 or 8 according to the invention and having essentially the same properties as the polypeptide as shown in the respective line in Table II, application no. 13, columns 5 or 8.

for the disclosure of this paragraph see [0359.1.7.7] above

A nucleic acid molecule encoding a homolog to a polypeptide depicted in the respective line in Table II, application no. 13, columns 5 or 8 can be created by introducing one or more nucleotide substitutions, additions or deletions into the nucleic acid molecule of the present invention, in particular of Table I, application no. 13, columns 5 or 8, in the respective line, such that one or more amino acid substitutions, additions or deletions are introduced into the encoded protein. Mutations can be introduced into the nucleic acid molecules as depicted in the respective line in Table I, application no. 13, columns 5 or 8, preferably the coding region thereof, by standard techniques, such as site-directed mutagenesis, PCR-mediated mutagenesis or other methods known to the person skilled in the art.

[0361.1.7.13] to [0363.1.7.13] for the disclosure of these paragraphs see [0361.1.7.7] to [0363.1.7.7] above

Homologues of the nucleic acid sequences used, with the sequences shown in the respective line in Table I, application no. 13, columns 5 or 8, preferably the coding region thereof, comprise also allelic variants with at least approximately 30%, 35%, 40%, 45%, 50%, 55%, 60%, 65%, 70%, 75%, 80%, 85%, 90%, 91%, 92%, 93%, 94% 95%. 96%, 97%, 98%, 99%, 99.5% or more % homology with one of the nucleotide sequences shown or the abovementioned derived nucleic acid sequences or their homologues or derivatives or parts of these. Allelic variants encompass in particular functional variants which can be obtained by deletion, insertion or substitution of nucleotides from the sequences shown in the respective line, preferably in Table I, columns 5 or 8, preferably the coding region thereof, or from the derived nucleic acid sequences, the intention being, however, that the enzymatic activity or the biological activity of the resulting proteins synthesized is advantageously retained or increased.

In one embodiment of the present invention, the nucleic acid molecule of the invention or used in the process of the invention comprises the sequences shown in the respective line in any of the Table I, application no. 13, columns 5 or 8, preferably the coding region thereof, fragments or homologs thereof. It is preferred that the nucleic acid molecule comprises as little as possible other nucleotides not shown in the respective line in any one of Table I, application no. 13, columns 5 or 8, preferably the coding region thereof, fragments or homologs thereof. In one embodiment, the nucleic acid molecule comprises less than 500, 400, 300, 200, 100, 90, 80, 70, 60, 50 or 40 further nucleotides. In a further embodiment, the nucleic acid molecule comprises less than 30, 20 or 10 further nucleotides. In one embodiment, the nucleic acid molecule used in the process of the invention is identical to the sequences shown in the respective line in Table I, application no. 13, columns 5 or 8, preferably the coding region thereof.

Also preferred is that the nucleic acid molecule used in the process of the invention encodes a polypeptide comprising the sequence shown in the respective line in Table II, application no. 13, columns 5 or 8, fragments or homologs thereof. In one embodiment, the nucleic acid molecule encodes less than 150, 130, 100, 80, 60, 50, 40 or 30 further amino acids. In a further embodiment, the encoded polypeptide comprises less than 20, 15, 10, 9, 8, 7, 6 or 5 further amino acids. In one embodiment used in the inventive process, the encoded polypeptide is identical to the sequences shown in the respective line in Table II, application no. 13, columns 5 or 8.

In one embodiment, the nucleic acid molecule of the invention or used in the process encodes a polypeptide comprising the sequence shown in the respective line in Table II, application no. 13, columns 5 or 8, or fragments or homologs thereof, and comprises less than 100 further nucleotides. In a further embodiment, said nucleic acid molecule comprises less than 30 further nucleotides. In one embodiment, the nucleic acid molecule used in the process is identical to a coding sequence of the sequences shown in the respective line in Table I, application no. 13, columns 5 or 8.

Polypeptides (=proteins), which still have the essential biological or enzymatic activity of the polypeptide of the present invention conferring the production or the increased production of the respective fine chemical abscisic acid, cryptoxanthin, violaxanthin, or zeaxanthin as compared to a corresponding, e.g. non-transformed, wild type non-human organism, like a microorganism or a plant cell, plant or part thereof, i.e. whose activity is essentially not reduced, are polypeptides with at least 10%, 20%, 30%, 40%, 50%, 60%, 70%, 80%, 90%, 95% or more of the wild type biological activity or enzymatic activity, advantageously, the activity is essentially not reduced in comparison with the activity of a polypeptide shown in the respective line in Table II, application no. 13, columns 5 or 8 expressed under identical conditions.

[0369.1.13.13] Homologues of nucleic acid molecules shown in the respective line in Table I, application no. 13, columns 5 or 8, preferably the coding region thereof, or fragments thereof, or of the derived sequences shown in the respective line in Table II, application no. 13, columns 5 or 8, or fragments thereof, also mean truncated sequences, cDNA, single-stranded DNA or RNA of the coding and noncoding DNA sequence. Homologues of said sequences are also understood as meaning derivatives, which comprise noncoding regions such as, for example, UTRs, introns, terminators, enhancers or promoter variants. The promoters upstream of the nucleotide sequences stated can be modified by one or more nucleotide substitution(s), insertion(s) and/or deletion(s) without, however, interfering with the functionality or activity either of the promoters, the open reading frame (=ORF) or with the 3′-regulatory region such as terminators or other 3′-regulatory regions, which are far away from the ORF. It is furthermore possible that the activity of the promoters is increased by modification of their sequence, or that they are replaced completely by more active promoters, even promoters from heterologous organisms.

Appropriate promoters are known to the person skilled in the art and are mentioned herein below.

[0370.1.7.13] to [0379.1.7.13] for the disclosure of these paragraphs see [0370.1.7.7] to [0379.1.7.7] above [0380.1.13.13] Moreover, a native polypeptide conferring the increase of the respective fine chemical abscisic acid, cryptoxanthin, violaxanthin, or zeaxanthin in a non-human organism or a part thereof can be isolated from cells (e.g., endothelial cells), for example using the antibody of the present invention as described below, in particular, an antibody against proteins having acetyl CoA carboxylase, acetyltransferase, acyl transferase, alcohol dehydrogenase, AX653549-protein, b1003-protein, b2032-protein, b2099-protein, b2121-protein, b2474-protein, b3346-protein, b3817-protein, coproporphyrinogen oxidase, F-box protein, flavodoxin, gluconate transport system permease 3, glutaredoxin, glycerol-3-phosphate dehydrogenase, glycine cleavage system T aminomethyltransferase, halotolerance protein, heat shock transcription factor, isopropylmalate isomerase large subunit, ketol-acid reductoisomerase, lipoprotein precursor, MADS-box transcription factor, melibiose carrier protein, monothiol glutaredoxin, monthiol glutaredoxin, N-acetylglucosaminylphosphatidylinositoldeacetylase, nitrate/nitrite transport protein, protein kinase, protein phosphatase, purine-nucleoside phosphorylase, ribosephosphate isomerase, constitutive, serine/threonine-protein phosphatase, sterol O-acyltransferase, superoxide dismutase, thioredoxin, transcription factor, transcriptional regulator, YCL026C-A-protein, YER184C-protein, ygl096w-protein, ymr324c-protein, or YOR044W-protein activity, respectively, or an antibody against polypeptides as shown in the respective line in Table II, application no. 13, columns 5 or 8, or fragments or homologs thereof which can be produced by standard techniques utilizing the polypeptid of the present invention or fragment thereof, i.e., the polypeptide of this invention (FCRP). Preferred are monoclonal antibodies.

for the disclosure of this paragraph see [0381.1.7.7] above

In one embodiment, the present invention relates to a polypeptide as depicted in the respective line in Table II, especially in Table IIA or especially in Table IIB, application no. 13, columns 5 or 8, or fragment or homolog thereof or as coded by the nucleic acid molecule shown in the respective line in Table I, especially Table IA or especially Table IB, application no. 13, columns 5 or 8, preferably the coding region thereof, or fragment or homolog thereof.

In an embodiment, the present invention relates to a polypeptide comprising or consisting of a consensus sequence shown in the respective line in Table IV, application no. 13, column 8 or at least one polypeptide motif shown in the respective line in Table IV, application no. 13, column 8.

In an embodiment thereof, the present invention relates to a polypeptide comprising or consisting of a consensus sequence shown in the respective line in Table IV, application no. 13, column 8 or comprising at least one polypeptide motif shown in the respective line in Table IV, application no. 13, column 8, whereby not more than 20, 15, 10, 9, 8, 7, 6, 5, 4, 3, 2, or 1, or 0 of the amino acids can be replaced by any amino acid.

In another embodiment thereof, the present invention relates to a polypeptide comprising or consisting of a consensus sequence shown in the respective line in Table IV, application no. 13, column 8 or comprising at least one polypeptide motif shown in the respective line in Table IV, application no. 13, column 8, whereby not more than 15%, 10%, 5%, 4%, 3%, 2% or1% or 0% of the amino acids defined as distinct specific amino acids are/is replaced by another amino acid.

In another embodiment thereof, the present invention relates to a polypeptide comprising or consisting of a consensus sequence shown in the respective line in Table IV, application no. 13, column 8 or comprising at least one polypeptide motif shown in the respective line in Table IV, application no. 13, column 8, whereby not more than 20, 15, 10, 9, 8, 7, 6, 5, 4, 3, 2, or 1, or 0 amino acids are inserted into the consensus sequence or polypeptide motif.

In an embodiment of the present invention, the polypeptide of the present invention comprises or consists of a consensus sequence or at least one polypeptide motif shown in the respective line in Table IV, application no. 13, column 8, whereby said polypeptide distinguishes over the sequence depicted in the respective line in Table II, application no. 13, columns 5 or 8 by one or more amino acids. In one embodiment, said polypeptide of the invention distinguishes over the sequence shown in the respective line in Table II, application no. 13, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 13, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in the respective line in Table II, application no. 13, columns 5 and 8.

In an embodiment, the invention relates to a polypeptide conferring an increase in the respective fine chemical abscisic acid, cryptoxanthin, violaxanthin, or zeaxanthin in a non-human organism, especially a microorganism or a plant, or a part thereof, being encoded by the nucleic acid molecule of the invention or used in the process of the invention and having a sequence which distinguishes over the sequence as shown in the respective line in Table II, application no. 13, columns 5 or 8 by one or more amino acids (but not exceeding 5%, preferably 4%, 3%, 2%, 1%, 0.5%, 0.2% or 0.1%). In an embodiment, said polypeptide of the invention does not comprise or consist of the sequence shown in the respective line in Table II, application no. 13, columns 5 or 8. In an embodiment, said polypeptide of the present invention is less than 100%, 99.999%, 99.99%, 99.9% or 99% identical. In one embodiment, said polypeptide which differs at least in one or more amino acids (but not exceeding 5%, preferably 4%, 3%, 2%, 1%, 0.5%, 0.2% or 0.1%) from the polypeptide shown in the respective line in Table II, application no. 13, columns 5 and 8 does not comprise a protein of the sequence shown in the respective line in Table II A and/or II B, application no. 13, columns 5 or 8.

In an embodiment, the present invention relates to a polypeptide having the activity of the protein as shown in the respective line in Table II, application no. 13, column 3, which distinguishes over the sequence depicted in the respective line in Table II, application no. 13, columns 5 or 8 by one or more amino acids, preferably by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 13, columns 5 and 8 by not more than 80%, 70%, 60%, 50%, 40%, 30%, 20%, 10%, 5%, 4%, 3%, 2%, 1% or 0.5%.

In another embodiment the polypeptide of the invention takes the form of a preprotein consisting of a plastidial or mitochondrial transit peptide joint to a polypeptide having the activity of the protein as shown in Table II, column 3, from which the transit peptide is preferably cleaved off upon transport of the preprotein into the organelle for example into the plastid or mitochondrion.

In another embodiment the polypeptide of the invention takes not the form of a preprotein.

for the disclosure of this paragraph see [0390.1.7.7] above

for the disclosure of this paragraph see [0391.1.7.7] above

A polypeptide of the invention can participate in the process of the present invention. The polypeptide or a portion thereof comprises preferably an amino acid sequence, which is sufficiently homologous to an amino acid sequence shown in the respective line in Table II A and/or II B, application no. 13, columns 5 or 8.

Further, the polypeptide can have an amino acid sequence which is encoded by a nucleotide sequence which hybridizes, preferably hybridizes under stringent conditions as described above, to a nucleotide sequence of the nucleic acid molecule of the present invention. Accordingly, the polypeptide has an amino acid sequence which is encoded by a nucleotide sequence that is at least about 35%, 40%, 45%, 50%, 55%, 60%, 65%, 70%, 75%, 80%, 85%, 90%, 91%, 92%, 93%, 94%, 95%, 96%, 97%, 98%, 99%, 99.5% or more homologous to one of the amino acid sequences as shown in the respective line in Table II A and/or II B, application no. 13, columns 5 or 8, or fragments thereof. The preferred polypeptide of the present invention preferably possesses at least one of the activities according to the invention and described herein. A preferred polypeptide of the present invention includes an amino acid sequence encoded by a nucleotide sequence which hybridizes, preferably hybridizes under stringent conditions, to a nucleotide sequence shown in the respective line in Table I A and/or I B, application no. 13, columns 5 or 8, preferably the coding region thereof, or fragments thereto or which is homologous thereto, as defined above

The polypeptide of the present invention can vary from the sequences shown in the respective line in Table II A and/or II B, application no. 13, columns 5 or 8 in amino acid sequence due to natural variation or mutagenesis, as described in detail herein. Accordingly, the polypeptide comprise an amino acid sequence which is at least about 35%, 40%, 45%, 50%, 55%, 60%, 65%, 70%, 75%, 80%, 85% or 90, 91%, 92%, 93%, 94%, 95%, 96%, 97%, 98%, 99%, 99.5% or more homologous to an entire amino acid sequence shown in the respective line in Table II A and/or II B, application no. 13, columns 5 or 8.

Biologically active portions of a polypeptide of the present invention include peptides comprising amino acid sequences derived from the amino acid sequence of the polypeptide of the present invention or used in the process of the present invention, e.g., the amino acid sequence shown in the respective line in Table II, application no. 13, columns 5 or 8 or the amino acid sequence of a protein homologous thereto, which include fewer amino acids than a full length polypeptide of the present invention or used in the process of the present invention or the full length protein which is homologous to a polypeptide of the present invention or used in the process of the present invention depicted herein, and exhibit at least one activity of a polypeptide of the present invention or used in the process of the present invention.

for the disclosure of this paragraph see [0396.1.7.7] above

Manipulation of the nucleic acid molecule of the invention may result in the production of a protein having differences from the protein as shown in the respective line in Table II, application no. 13, column 5 or 8. Differences shall mean at least one amino acid different from the sequences as shown in the respective line in Table II, application no. 13, column 5 or 8, preferably at least 2, 3, 4, 5, 6, 7, 8, 9 or 10 amino acids (especially but not exceeding 0.1%, preferably 0.2%, 0.5%, 1%, 2%, 3% 4% or 5%), more preferably at least 15, 20, 25, 30, 35, 40, 45 or 50 amino acids (especially but not exceeding 0.5%, preferably 1%, 2%, 3% 4%, 5%, 10% or 25%) different from the sequences as shown in the respective line in Table II, application no. 13, column 5 or 8. These proteins may be improved in efficiency or activity, or may be more stable and therefore present in greater numbers in the cell as compared to the wild-type cell.

Any mutagenesis strategies for the polypeptide of the present invention or the polypeptide used in the process of the present invention to result in increasing said activity are not meant to be limiting; variations on these strategies will be readily apparent to one skilled in the art. Using such strategies, and incorporating the mechanisms disclosed herein, the nucleic acid molecule and polypeptide of the invention may be utilized to generate algae, ciliates, fungi, other microorganisms like C. glutamicum or plants or parts thereof, expressing proteins as shown in the respective line in Table II, application no. 13, column 5 or 8, or mutated proteins thereof. The nucleic acid molecules and polypeptide molecules of the invention are expressed such that the yield, production, and/or efficiency of production of a desired compound is improved.

[0399.1.7.13] to [0401.1.7.13] for the disclosure of these paragraphs see [0399.1.7.7] to [0401.1.7.7] above

In one embodiment, a protein (=polypeptide) as shown in the respective line in Table II, application no. 13, column 5 or 8 refers to a polypeptide having an amino acid sequence corresponding to the polypeptide of the invention, especially to a polypeptide as shown in Table II, application no. 13, columns 5 or 8 or homologs thereof, or used in the process of the invention, whereas a “non-inventive protein or polypeptide” or “other polypeptide” refers to a polypeptide having an amino acid sequence corresponding to a protein which is not substantially homologous to a polypeptide of the invention, preferably which is not substantially homologous to a polypeptide or protein as shown in the respective line in Table II, application no. 13, column 5 or 8 or homologs thereof, e.g., a protein which does not confer the activity described herein and which is derived from the same or a different organism.

[0403.1.7.13] to [0409.1.7.13] for the disclosure of these paragraphs see [0403.1.7.7] to [0409.1.7.7] above

As the nucleic acid molecules of the invention and/or the expression thereof is related to the synthesis of the respective fine chemical abscisic acid, cryptoxanthin, violaxanthin, or zeaxanthin its function as a probe extends to the detection of microorganisms, plant tissues, plants, plant variets, plant ecotypes or plant genera with varying, advantageously increased, capability or potential for synthesis of the respective fine chemical abscisic acid, cryptoxanthin, violaxanthin, or zeaxanthin. Therefore in one embodiment the present invention relates to a method for analyzing the capability or potential of a plant tissue, a plant, a plant variety or ecotype to produce the respective fine chemical abscisic acid, cryptoxanthin, violaxanthin, or zeaxanthin by using the respective antibody of the invention as a probe to detect the amount of the polypeptide encoded by said nucleic acid molecule of the invention in a non-human organism in comparison to another organism.

Therefore in one embodiment the invention relates to a method to probe a non-human organism for its capability for the production of the respective fine chemical comprising the steps of
a) analyzing the sequence and/or the expression of the respective nucleic acid molecule of the invention in least two non-human organisms which differ in the capability to produce the respective fine chemical;
b) associate the different sequence and/or expression level of the nucleic acid molecules of the invention with their different capability to produce the respective fine chemical;
c) analyze the sequence and/or the expression of the nucleic acid molecule of the invention in at least a third non-human organism;
d) predict the capability of the at least third organism to produce the respective fine chemical based by comparing the sequence and/or expression of the nucleic acid of the invention to the sequences and/or expression levels of the nucleic acid molecule of the invention of the at least two organisms analyzed in step a.

[0411.1.7.13] to [0430.1.7.13] for the disclosure of these paragraphs see [0411.1.7.7] to [0430.1.7.7] above

Accordingly the present invention relates to any cell transgenic for any nucleic acid characterized as part of the invention, e.g. conferring the increase of the respective fine chemical abscisic acid, cryptoxanthin, violaxanthin, or zeaxanthin in a cell or a non-human organism or a part thereof, e.g. the nucleic acid molecule of the invention, the nucleic acid construct of the invention, the vector of the invention, the expression cassette according to the invention, or a nucleic acid molecule encoding the polypeptide of the invention, e.g. encoding a polypeptide having an activity as the protein as shown in the respective line in Table II, application no. 13, column 3. Due to the above-mentioned activity the respective fine chemical abscisic acid, cryptoxanthin, violaxanthin, or zeaxanthin content in a cell or a non-human organism is increased. For example, due to modulation or manipulation, the cellular activity is increased, in a preferred embodiment in organelles such as plastids or mitochondria, e.g. due to an increased expression or specific activity or specific targeting of the subject matters of the invention in a cell or a non-human organism or a part thereof especially in organelles such as plastids or mitochondria, or in another embodiment in the cytosol. Transgenic for a polypeptide having a protein or a protein activity means herein that due to modulation or manipulation of the genome, the activity of protein as shown in the respective line in Table II, application no. 13, column 3 or a protein as shown in the respective line in Table II, application no. 13, column 3-like activity is increased in the cell or non-human organism or part thereof, especially in organelles such as plastids or mitochondria, or especially in the cytosol. Examples are described above in context with the process of the invention.

for the disclosure of this paragraph see [0432.1.7.7] above

A naturally occurring expression cassette—for example the naturally occurring combination of the promoter of the gene encoding a protein as shown in the respective line in Table II, application no. 13, column 5 or 8, preferably the coding region thereof, or fragments or homologs thereof with the corresponding encoding gene—becomes a transgenic expression cassette when it is modified by non-natural, synthetic “artificial” methods such as, for example, mutagenesis. Such methods have been described (U.S. Pat. No. 5,565,350; WO 00/15815; also see above).

[0434.1.7.13] for the disclosure of this paragraph see [0434.1.7.7] above

for the disclosure of this paragraph see [0435.1.7.7] above

Transgenic plants comprising the respective fine chemical(s) synthesized in the process according to the invention can be marketed directly without isolation of the fine chemical synthesized. In the process according to the invention, plants are understood as meaning all plant parts, plant organs such as leaf, stalk, root, tubers or seeds or propagation material or harvested material or the intact plant. In this context, the seed encompasses all parts of the seed such as the seed coats, epidermal cells, seed cells, endosperm or embryonic tissue. The fine chemical produced in the process according to the invention may, however, also be isolated from the plant. In case of abscisic acid, cryptoxanthin, violaxanthin, or zeaxanthin this can be in free form or bound to proteins. Fine chemical(s) produced by this process can be harvested by harvesting the non-human organisms either from the culture in which they grow or from the field. For example, this can be done via squeezing, grinding and/or extraction, salt precipitation and/or ion-exchange chromatography of the plant parts, preferably the plant seeds, plant fruits, plant tubers and the like.

[0437.1.7.13] to [0447.1.7.13] for the disclosure of these paragraphs see [0437.1.7.7] to [0447.1.7.7] above

The identity and purity of the compound(s) isolated can be determined by priorganism art techniques. They encompass high-performance liquid chromatography (HPLC), gas chromatography (GC), spectroscopic methods, mass spectrometry (MS), staining methods, thin-layer chromatography, NIRS, enzyme assays or microbiological assays. These analytical methods are compiled in: Patek et al. (1994) Appl. Environ. Microbiol. 60:133-140; Malakhova et al. (1996) Biotekhnologiya 11 27-32; and Schmidt et al. (1998) Bioprocess Engineer. 19:67-70. Ulmann's Encyclopedia of Industrial Chemistry (1996) Bd. A27, VCH Weinheim, pp. 89-90, pp. 521-540, pp. 540-547, pp. 559-566, 575-581 and pp. 581-587; Michal, G (1999) Biochemical Pathways: An Atlas of Biochemistry and Molecular Biology, John Wiley and Sons; Fallon, A. et al. (1987) Applications of HPLC in Biochemistry in: Laboratory Techniques in Biochemistry and Molecular Biology, vol. 17.

Xanthophylls, in particular beta-cryptoxanthin or zeaxanthin can for example be detected advantageously via HPLC, LC or GC separation methods. The unambiguous detection for the presence of xanthophylls, in particular beta-cryptoxanthin or zeaxanthin containing products can be obtained by analyzing recombinant organisms using analytical standard methods: LC, LC-MS, MS or TLC). The material to be analyzed can be disrupted by sonication, grinding in a glass mill, liquid nitrogen and grinding, cooking, or via other applicable methods

For HPLC, e.g. chromatograph on Spherisorb ODS-2 columns with a solvent system consisting of water, acetonitrile and and isopropanol and detection at 436 nm can be used (Li and Walton, Plant Physiol. (1990) 92, 551-559), specially for violaxanthin.

The xanthophylls, in particular the respective fine chemicals obtained in the process are suitable as starting material for the synthesis of further products of value. For example, they can be used in combination with each other or alone for the production of pharmaceuticals, health products, foodstuffs, animal feeds, nutrients or cosmetics. Accordingly, the present invention relates a method for the production of pharmaceuticals, health products, food stuff, animal feeds, nutrients or cosmetics comprising the steps of the process according to the invention, including the isolation of the carotenoids containing, in particular xanthophylls and/or abcisic acid containing composition produced or the respective fine chemical produced if desired and formulating the product with a pharmaceutical acceptable carrier or formulating the product in a form acceptable for an application in agriculture. A further embodiment according to the invention is the use of the carentoids or xanthophylls or abcisic acid produced in the process or of the transgenic organisms in animal feeds, foodstuffs, medicines, food supplements, cosmetics or pharmaceuticals or for the production of astaxanthin, e.g. in after isolation of the respective fine chemical or without, e.g. in situ, e.g in the organism used for the process for the production of the respective fine chemical.

[0450.1.7.13] to [0452.1.7.13] for the disclosure of these paragraphs see [0450.1.7.7] to [0452.1.7.7] above

Further in another embodiment, the present invention relates to the use of the nucleic acid molecule of the invention or used in the method of the invention alone or in combination with other genes of the respective fine chemical synthesis for example of the carotenoids, preferably xanthophylls or abcisic acid resp., in particular, of abscisic acid, cryptoxanthin, violaxanthin, and/or zeaxanthin biosynthesis, the polypeptide of the invention or used in the method of the invention, the nucleic acid construct of the invention, the expression cassette of the invention, the vector of the invention, the plant or plant tissue or the host cell of the invention, for the production of plant resistant to a herbicide inhibiting eventually the production of the fine chemical.

for the disclosure of this paragraph see [0454.1.7.7] above

In one embodiment, the present invention relates to a method for the identification of a gene product conferring the production of or an increase in the fine chemical production in a non-human cell, comprising the following steps:
(a) contacting, e.g. hybridizing, a, some or all nucleic acid molecules of a sample, e.g. cells, tissues, plants or microorganisms or a nucleic acid library, which can contain a candidate gene encoding a gene product conferring the production of or an increase in the fine chemical after expression, with the nucleic acid molecule of the present invention as shown in the respective line in column 5 or 8 of Table I A or B of application no. 13, preferably the coding region thereof, or a fragment or homolog thereof;
(b) identifying the nucleic acid molecules, which hybridize under relaxed stringent conditions with the nucleic acid molecule of the present invention in particular to the nucleic acid molecule sequence shown in the respective line in Table I, application no. 13, columns 5 or 8, preferably the coding region thereof, of a fragment or a homolog thereof, preferably in Table I B, application no. 13, columns 5 or 8, preferably the coding region thereof, of a fragment or a homolog thereof, and, optionally, isolating the full length cDNA clone or complete genomic clone;
(c) introducing the candidate nucleic acid molecules or a fragment thereof in host cells, preferably in a plant cell or a microorganism, appropriate for producing the fine chemical;
(d) expressing the identified nucleic acid molecules in the host cells for which the production of or the increased production of the fine chemical is desired;
(e) assaying the fine chemical level in the non-human host cells; and
(f) identifying the nucleic acid molecule and/or its gene product which expression confers the production of or an increase in the fine chemical level in the non-human host cell after expression compared to the wild type.

for the disclosure of this paragraph see [0456.1.7.7] above

In another embodiment, the present invention relates to a method for the identification of a gene product conferring the production of or an increase in the fine chemical production in a cell, comprising the following steps:
(a) identifying a nucleic acid molecule of a non-human organism, which is at least 20%, 25%, 30%, 35%, 40%, 50%, 60%, 70%, 80%, 90%, 95%, 96%, 97%, 98%, 99%, 99.5% or more homolog to the nucleic acid molecule encoding a polypeptide comprising the polypeptide molecule as shown in the respective line in column 5 or 8 of Table II, application no. 13, or comprising a consensus sequence or a polypeptide motif as shown in the respective line in column 8 of Table IV, application no. 13, or being encoded by a nucleic acid molecule comprising a polynucleotide as shown in the respective line in column 5 or 8 of Table I, application no. 13, preferably the coding region thereof, or a homologue thereof as described herein, for example via homology search in a data bank;
(b) introducing the candidate nucleic acid molecule in non-human host cells, preferably in a plant cell or a microorganism, appropriate for producing the fine chemical;
(c) expressing the identified nucleic acid molecule in the non-human host cells;
(d) assaying the fine chemical level in the non-human host cells; and
(e) identifying the nucleic acid molecule and its gene product which expression confers the production of or an increase in the fine chemical level in the non-human host cell after expression compared to the wild type.

for the disclosure of this paragraph see [0458.1.7.7] above

Further, the nucleic acid molecules disclosed herein, in particular the nucleic acid molecules shown in the respective line in column 5 or 8 of Table I A or B of application no. 13, may be sufficiently homologous to the sequences of related species such that these nucleic acid molecules may serve as markers for the construction of a genomic map in related organism or for association mapping. Furthermore natural variation in the genomic regions corresponding to nucleic acids disclosed herein, in particular the nucleic acid molecule shown in the respective line in column 5 or 8 of Table I A or B, of application no. 13 or homologous thereof, may lead to variation in the activity of the proteins disclosed herein, in particular the proteins comprising polypeptides as shown in the respective lines in column 5 or 8 of Table II A or B, of application no. 13 or their homologous, or comprising the consensus sequence or the polypeptide motif as shown in the respective line in column 8 of Table IV, and in consequence in a natural variation of an increased fine chemical production.

In consequence natural variation eventually also exists in form of more active allelic variants leading already to a relative increase in the fine chemical. Different variants of the nucleic acid molecules disclosed herein, in particular the nucleic acid molecules comprising the nucleic acid molecules as shown in the respective line in column 5 or 8 of Table I A or B of application no. 13, preferably the coding region thereof, which corresponds to different levels of increase in fine chemical, e.g. different levels of increase in fine chemical, can be indentified and used for marker assisted breeding for an enhanced production of the fine chemical.

Natural variation may also exist in the regulatory regions, e.g. the promoters of the natural genes comprising the nucleic acid molecules as shown in the respective line column 5 or 8 of Table I A or B of application no. 13, preferably the coding region thereof, which can similarly correspond to different levels of increase in the fine chemical, e.g. different levels of increase in the fine chemical can be identified and used for marker assisted breeding for an enhanced production of the fine chemical.

Accordingly, the present invention relates to a method for breeding plants for the production of the fine chemical, comprising
(a) selecting a first plant variety with an increased production of the fine chemical, e.g. based on an increased expression of a nucleic acid molecule of the invention as disclosed herein, in particular of a nucleic acid molecule comprising a nucleic acid molecule as shown in the respective line in column 5 or 8 of Table I A or B of application no. 13, preferably the coding region thereof, or fragments or homologs thereof, or a polypeptide comprising a polypeptide as shown in the respective line in column 5 or 8 of Table II A or B of application no. 13, or fragments or homologs thereof, or comprising a consensus sequence or a polypeptide motif as shown in the respective line in column 8 of Table IV, or a homolog thereof, as described herein;
(b) associating the level of increased production of the fine chemical, with the expression level or the genomic structure of a gene encoding said polypeptide or said nucleic acid molecule;
(c) crossing the first plant variety with a second plant variety, which significantly differs in its level of fine chemical; and
(d) identifying, which of the offspring varieties has got increased production of the fine chemical by the expression level of said polypeptide or nucleic acid molecule or the genomic structure of the gene(s) encoding said polypeptide or nucleic acid molecule of the invention.

[0462.1.7.13] to [0479.1.7.13] for the disclosure of these paragraphs see [0462.1.7.7] to [0479.1.7.7] above

In a further embodiment the nucleic acid molecule of the invention, the polypeptide of the invention, the nucleic acid construct of the invention, the expression cassette of the invention, the non-human organisms, the non-human host cell, the microorganisms, the plant, plant cell, or the part thereof, of the invention, the vector of the invention, the agonist identified with the method of the invention, the nucleic acid molecule identified with the method of the present invention, can be used for the production of the fine chemical or of the fine chemical and one or more other carotenoids, preferably zeaxanthin (E161h, used for food coloring) capsanthin (E160c, used for food coloring), astaxanthin (E 161j, used as food suplement for human diet and in fish breeding; used also in sun-protectives) or lutein (E 161b, used for food coloring).

Accordingly, the nucleic acid molecule of the invention, or the nucleic acid molecule identified with the method of the present invention or the complement sequences thereof, the polypeptide of the invention, the nucleic acid construct of the invention, the expression cassette of the invention, the non-human organism, the non-human host cell, the microorganism, the plant, plant cell, or the part thereof of the invention, the vector of the invention, the agonist identified with the method of the invention, the antibody of the present invention, can be used for the production of or the increase in the fine chemical in a non-human organism or part thereof, e.g. in a cell.

for the disclosure of this paragraph see [0481.1.7.7] above

for the disclosure of this paragraph see [0482.1.7.7] above

A special embodiment of this invention is related to genes which increase or generate the production of the fine chemical abscisic acid in plant cells, plants or part thereof. Phenotypes thereto are associated with yield of plants (=yield related phenotypes). In accorganism dance with the invention, therefore, the respective genes identified in Table I, wherein in column 7 abscisic acid are mentioned, especially the coding region thereof, or homologs or fragments thereof, may be employed to enhance any yield-related phenotype. Increased yield may be determined in field trials of transgenic plants and suitable control plants. Alternatively, a transgene's ability to increase yield may be determined in a model plant. An increased yield phenotype may be determined in the field test or in a model plant by measuring any one or any combination of the following phenotypes, in comparison to a control plant: yield of dry harvestable parts of the plant, yield of dry aerial harvestable parts of the plant, yield of underground dry harvestable parts of the plant, yield of fresh weight harvestable parts of the plant, yield of aerial fresh weight harvestable parts of the plant yield of underground fresh weight harvestable parts of the plant, yield of the plant's fruit (both fresh and dried), grain dry weight, yield of seeds (both fresh and dry), and the like.

The most basic yield-related phenotype is increased yield associated with the presence of the gene or a homolog or a fragment thereof as a transgene in the plant, i.e., the intrinsic yield of the plant. Intrinsic yield capacity of a plant can be, for example, manifested in a field test or in a model system by demonstrating an improvement of seed yield (e.g. in terms of increased seed/grain size, increased ear number, increased seed number per ear, improvement of seed filling, improvement of seed composition, embryo and/or endosperm improvements, and the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under non-stressed conditions), improvement in plant architecture.

Increased yield-related phenotypes may also be measured to determine tolerance to abiotic environmental stress. Abiotic stresses include drought, low temperature, salinity, osmotic stress, shade, high plant density, mechanical stresses, and oxidative stress, and yield-related phenotypes are encompassed by tolerance to such abiotic stresses. Additional phenotypes that can be monitored to determine enhanced tolerance to abiotic environmental stress include, without limitation, wilting; leaf browning; loss of turgor, which results in drooping of leaves or needles stems, and flowers; drooping and/or shedding of leaves or needles; the leaves are green but leaf angled slightly toward the ground compared with controls; leaf blades begun to fold (curl) inward; premature senescence of leaves or needles; loss of chlorophyll in leaves or needles and/or yellowing. Any of the yield-related phenotypes described above may be monitored in field tests or in model plants to demonstrate that a transgenic plant has increased tolerance to abiotic environmental stress. In accordance with the invention, the respective genes identified in Table 1, in case in column 7 abscisic acid is indicated, especially the coding region thereof, or homologs or fragments thereof, may be employed to enhance tolerance to abiotic environmental stress in a plant means that the plant, when confronted with abiotic environmental stress.

A polypeptide conferring an yield-increasing activity can be encoded by a respective nucleic acid sequence as shown in Table I, column 5 or 8, and/or comprises or consists of a respective polypeptide as depicted in Table II, column 5 and 8, and/or can be amplified with the respective primer set shown in Table III, column 8, in case in column 7 abscisic acid is indicated. “Improved adaptation” to environmental stress like e.g. freezing and/or chilling temperatures refers to an improved plant performance under environmental stress conditions.

A modification, i.e. an increase, can be caused by endogenous or exogenous factors. For example, an increase in activity in an organism or a part thereof can be caused by adding a gene product or a precursor or an activator or an agonist to the media or nutrition or can be caused by introducing said subjects into an organism, transient or stable. Furthermore such an increase can be reached by the introduction of the respective inventive nucleic acid sequence or the encoded protein in the correct cell compartment for example into the nucleus or cytoplasmic respectively or into plastids either by transformation and/or targeting.

The term “yield” as used herein generally refers to a measurable produce from a plant, particularly a crop. Yield and yield increase (in comparison to a non-transformed starting or wild-type plant) can be measured in a number of ways, and it is understood that a skilled person will be able to apply the correct meaning in view of the particular embodiments, the particular crop concerned and the specific purpose or application concerned. The terms “improved yield” or “increased yield” can be used interchangeable.

As used herein, the term “improved yield” or the term “increased yield” means any improvement in the yield of any measured plant product, such as grain, fruit or fiber. In accordance with the invention, changes in different phenotypic traits may improve yield. For example, and without limitation, parameters such as floral organ development, root initiation, root biomass, seed number, seed weight, harvest index, tolerance to abiotic environmental stress, leaf formation, phototropism, apical dominance, and fruit development, are suitable measurements of improved yield. Any increase in yield is an improved yield in accordance with the invention. For example, the improvement in yield can comprise a 0.1%, 0.5%, 1%, 3%, 5%, 10%, 15%, 20%, 30%, 40%, 50%, 60%, 70%, 80%, 90% or greater increase in any measured parameter. For example, an increase in the bushel/acre yield of soybeans or corn derived from a crop comprising plants which are transgenic for the nucleotides and polypeptides of Table I, in case in column 7 abscisic acid is mentioned, as compared with the bushel/acre yield from untreated soybeans or corn cultivated under the same conditions, is an improved yield in accordance with the invention. The increased or improved yield can be achieved in the absence or presence of stress conditions.

For example, enhanced or increased “yield” refers to one or more yield parameters selected from the group consisting of biomass yield, dry biomass yield, aerial dry biomass yield, underground dry biomass yield, fresh-weight biomass yield, aerial fresh-weight biomass yield, underground fresh-weight biomass yield; enhanced yield of harvestable parts, either dry or freshweight or both, either aerial or underground or both; enhanced yield of crop fruit, either dry or fresh-weight or both, either aerial or underground or both; and preferably enhanced yield of seeds, either dry or fresh-weight or both, either aerial or underground or both.

Crop yield is defined herein as the number of bushels of relevant agricultural product (such as grain, forage, or seed) harvested per acre. Crop yield is impacted by abiotic stresses, such as drought, heat, salinity, and cold stress, and by the size (biomass) of the plant. Traditional plant breeding strategies are relatively slow and have in general not been successful in conferring increased tolerance to abiotic stresses. Grain yield improvements by conventional breeding have nearly reached a plateau in maize.

Accordingly, the yield of a plant can depend on the specific plant/crop of interest as well as its intended application (such as food production, feed production, processed food production, biofuel, biogas or alcohol production, or the like) of interest in each particular case. Thus, in one embodiment, yield is calculated as harvest index (expressed as a ratio of the weight of the respective harvestable parts divided by the total biomass), harvestable parts weight per area (acre, square meter, or the like); and the like. The harvest index, i.e., the ratio of yield biomass to the total cumulative biomass at harvest, in maize has remained essentially unchanged during selective breeding for grain yield over the last hundred years. Accordingly, recent yield improvements that have occurred in maize are the result of the increased total biomass production per unit land area. This increased total biomass has been achieved by increasing planting density, which has led to adaptive phenotypic alterations, such as a reduction in leaf angle, which may reduce shading of lower leaves, and tassel size, which may increase harvest index. Harvest index is relatively stable under many environmental conditions, and so a robust correlation between plant size and grain yield is possible. Plant size and grain yield are intrinsically linked, because the majority of grain biomass is dependent on current or stored photosynthetic productivity by the leaves and stem of the plant. As with abiotic stress tolerance, measurements of plant size in early development, under standardized conditions in a growth chamber or greenhouse, are standard practices to measure potential yield advantages conferred by the presence of a transgene.

Acccordingly, the yield of a plant can be increased by improving one or more of the yield-related phenotypes.

Such yield-related phenotypes or traits of a plant the improvement of which results in increased yield comprise, without limitation, the increase of the intrinsic yield capacity of a plant, improved nutrient use efficiency, and/or increased stress tolerance.

For example, yield refers to biomass yield, e.g. to dry weight biomass yield and/or fresh-weight biomass yield. Biomass yield refers to the aerial or underground parts of a plant, depending on the specific circumstances (test conditions, specific crop of interest, application of interest, and the like). In one embodiment, biomass yield refers to the aerial and underground parts. Biomass yield may be calculated as fresh-weight, dry weight or a moisture adjusted basis. Biomass yield may be calculated on a per plant basis or in relation to a specific area (e.g. biomass yield per acre/ square meter/ or the like).

In other embodiment, “yield” refers to seed yield which can be measured by one or more of the following parameters: number of seeds or number of filled seeds (per plant or per area (acre/square meter/ or the like)); seed filling rate (ratio between number of filled seeds and total number of seeds); number of flowers per plant; seed biomass or total seeds weight (per plant or per area (acre/square meter/ or the like); thousand kernel weight (TKW; extrapolated from the number of filled seeds counted and their total weight; an increase in TKW may be caused by an increased seed size, an increased seed weight, an increased embryo size, and/or an increased endosperm). Other parameters allowing to measure seed yield are also known in the art. Seed yield may be determined on a dry weight or on a fresh weight basis, or typically on a moisture adjusted basis, e.g. at 15.5 percent moisture.

For example, the term “increased yield” means that the plant, exhibits an increased growth rate, under conditions of abiotic environmental stress, compared to the corresponding wild-type plant.

An increased growth rate may be reflected inter alia by or confers an increased biomass production of the whole plant, or an increased biomass production of the aerial parts of a plant, or by an increased biomass production of the underground parts of a plant, or by an increased biomass production of parts of a plant, like stems, leaves, blossoms, fruits, and/or seeds.

Increased yield includes higher fruit yields, higher seed yields, higher fresh matter production, and/or higher dry matter production.

The term “increased yield” means that the plant, exhibits a prolonged growth under conditions of abiotic environmental stress, as compared to the corresponding, e.g. non-transformed, wild type organism. A prolonged growth comprises survival and/or continued growth of the plant, at the moment when the non-transformed wild type organism shows visual symptoms of deficiency and/or death.

When the plant of the invention is a corn plant increased yield for corn plants means, for example, increased seed yield, in particular for corn varieties used for feed or food. Increased seed yield of corn refers to an increased kernel size or weight, an increased kernel per ear, or increased ears per plant. Alternatively or in addition the cob yield may be increased, or the length or size of the cob is increased, or the kernel per cob ratio is improved.

When the plant of the invention is a soy plant, increased yield for soy plants means increased seed yield, in particular for soy varieties used for feed or food. Increased seed yield of soy refers for example to an increased kernel size or weight, an increased kernel per pod, or increased pods per plant.

When the plant of the invention is an oil seed rape (OSR) plant, increased yield for OSR plants means increased seed yield, in particular for OSR varieties used for feed or food. Increased seed yield of OSR refers to an increased seed size or weight, an increased seed number per silique, or increased siliques per plant.

When the plant of the invention is a cotton plant,increased yield for cotton plants means increased lint yield. Increased lint yield of cotton refers in one embodiment to an increased length of lint.

When the plant of the invention is a wheat plant increased yield for wheat plants means, for example, increased seed yield, in particular for wheat varieties used for feed or food. Increased seed yield of wheat refers to an increased kernel size or weight, an increased kernel per ear, or increased ears per plant. Increased seed yield of wheat refers to an increased grain size or weight, an increased number of grains per spike, or an increased number of spikes per plant. Additionally or as an alternative increased seed yield of wheat refers to an increased 1000 grain weight, an increased length of spikes, or an increased number of spikelets per spikes, or an increased number of spikes per square meter, or or an increased flag leaf area (flag leaf is the leaf directly below the spike).

Said increased yield can typically be achieved by enhancing or improving, one or more yieldrelated traits of the plant. Such yield-related traits of a plant comprise, without limitation, the increase of the intrinsic yield capacity of a plant, improved nutrient use efficiency, and/or increased stress tolerance, in particular increased abiotic stress tolerance.

Intrinsic yield capacity of a plant can be, for example, manifested by improving the specific (intrinsic) seed yield (e.g. in terms of increased seed/ grain size, increased ear number, increased seed number per ear, improvement of seed filling, improvement of seed composition, embryo and/or endosperm improvements, or the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under stressed or non-stressed conditions), improvement in plant architecture, cell cycle modifications, photosynthesis modifications, various signaling pathway modifications, modification of transcriptional regulation, modification of translational regulation, modification of enzyme activities, and the like); and/or the like.

The improvement or increase of stress tolerance of a plant can for example be manifested by improving or increasing a plant's tolerance against stress, particularly abiotic stress. In the present application, abiotic stress refers generally to abiotic environmental conditions a plant is typically confronted with, including, but not limited to, drought (tolerance to drought may be achieved as a result of improved water use efficiency), heat, low temperatures and cold conditions (such as freezing and chilling conditions), salinity, osmotic stress , shade, high plant density, mechanical stress, oxidative stress, and the like.

The increased plant yield can also be mediated by increasing the “nutrient use efficiency of a plant”, e.g. by improving the use efficiency of nutrients including, but not limited to, phosphorus, potassium, and nitrogen. Further, higher yields may be obtained with current or standard levels of nitrogen use.

Generally, the term “increased tolerance to stress” can be defined as survival of plants, and/or higher yield production, under stress conditions as compared to a non-transformed wild type or starting plant: For example, the plant of the invention or produced according to the method of the invention is better adapted to the stress conditions. “Improved adaptation” to environmental stress like e.g. drought, heat, nutrient depletion, freezing and/or chilling temperatures refers herein to an improved plant performance resulting in an increased yield, particularly with regard to one or more of the yield related traits as defined in more detail above.

During its life-cycle, a plant is generally confronted with a diversity of environmental conditions. Any such conditions, which may, under certain circumstances, have an impact on plant yield, are herein referred to as “stress” condition. Environmental stresses may generally be divided into biotic and abiotic (environmental) stresses. Unfavorable nutrient conditions are sometimes also referred to as “environmental stress”. The present invention does also contemplate solutions for this kind of environmental stress, e.g. referring to increased nutrient use efficiency.

For the purposes of the description of the present invention, the terms “enhanced tolerance to abiotic stress”, “enhanced resistance to abiotic environmental stress”, “enhanced tolerance to environmental stress”, “improved adaptation to environmental stress” and other variations and expressions similar in its meaning are used interchangeably and refer, without limitation, to an improvement in tolerance to one or more abiotic environmental stress(es) as described herein and as compared to a corresponding origin or wild type plant or a part thereof. The term abiotic stress tolerance(s) refers for example low temperature tolerance, drought tolerance or improved water use efficiency (WUE), heat tolerance, salt stress tolerance and others. Studies of a plant's response to desiccation, osmotic shock, and temperature extremes are also employed to determine the plant's tolerance or resistance to abiotic stresses. Water use efficiency (WUE) is a parameter often correlated with drought tolerance. In selecting traits for improving crops, a decrease in water use, without a change in growth would have particular merit in an irrigated agricultural system where the water input costs were high. An increase in growth without a corresponding jump in water use would have applicability to all agricultural systems. In many agricultural systems where water supply is not limiting, an increase in growth, even if it came at the expense of an increase in water use also increases yield.

Drought stress means any environmental stress which leads to a lack of water in plants or reduction of water supply to plants, including a secondary stress by low temperature and/or salt, and/or a primary stress during drought or heat, e.g. desiccation etc.

Accordingly, this invention provides respective measures and methods to produce plants with increased yield, e.g. genes conferring an increased yield-related trait, for example enhanced tolerance to abiotic environmental stress, for example an increased drought tolerance and/or low temperature tolerance, intrinsic yield and/or another increased yield-related trait, upon expression or over-expression. Accordingly, the present invention provides such genes in case in column 7 of the respective Table or abscisic acid is indicated. In particular, such genes are described in column 5 as well as in column 8 of Tables I, especially the coding region thereof, or homologs or fragments thereof, in case abscisic acid are indicated in column 7 or the respective polypeptides are described in column 5 as well as in column 8 of Table II, or homologs or fragments thereof, in case abscisic acid are indicated in column 7.

Accordingly, the present invention provides respective transgenic plants showing one or more improved yield-related traits as compared to the corresponding origin or the wild type plant and methods for producing such transgenic plants with increased yield in case in column 7 of the respective Table “abscisic acid” is indicated.

In one embodiment, one or more of said yield-increasing activities are increased by increasing the amount and/or the specific activity of one or more proteins listed in Table II, column 5 or 8 in a compartment of a cell indicated in Table II, column 6, in case in column 7 of the respective Table “abscisic acid” is indicated.

Accordingly to present invention, the yield of the plant of the invention is increased by improving one or more of the yield-related traits as defined herein. Said increased yield in accordance with the present invention can typically be achieved by enhancing or improving, in comparison to an origin or wild-type plant, one or more yield-related traits of said plant. Such yield-related traits of a plant the improvement of which results in increased yield comprise, without limitation, the increase of the intrinsic yield capacity of a plant, improved nutrient use efficiency, and/or increased stress tolerance, preferably an increased drought tolerance.

In one embodiment, the present invention provides respective transgenic plants showing one or more improved yield-related traits, preferably increased drought tolerance as compared to the corresponding origin or the wild type plant and methods for producing such transgenic plants with increased yield in case in column 7 of the respective Table “abscisic acid” is indicated.

In a further embodiment the present invention is characterized by the features as disclosed in the following items:

Item 1. A process for the production of a fine chemical selected from the group consisting of: abscisic acid, cryptoxanthin, violaxanthin and/or zeaxanthin
(a) increasing or generating one or more activities selected from the group consisting of acetyl CoA carboxylase, acetyltransferase, acyl transferase, alcohol dehydrogenase, AX653549-protein, b1003-protein, b2032-protein, b2099-protein, b2121-protein, b2474-protein, b3346-protein, b3817-protein, coproporphyrinogen oxidase, F-box protein, flavodoxin, gluconate transport system permease 3, glutaredoxin, glycerol-3-phosphate dehydrogenase, Glycine cleavage system T aminomethyltransferase, halotolerance protein, heat shock transcription factor, isopropylmalate isomerase large subunit, ketol-acid reductoisomerase, lipoprotein precursor, MADS-box transcription factor, melibiose carrier protein, monothiol glutaredoxin, monthiol glutaredoxin, N-acetylglucosaminylphosphatidylinositoldeacetylase, nitrate/nitrite transport protein, protein kinase, protein phosphatase, purine-nucleoside phosphorylase, ribosephosphate isomerase, constitutive, serine/threonine-protein phosphatase, sterol O-acyltransferase, superoxide dismutase, thioredoxin, transcription factor, transcriptional regulator, YCL026C-A-protein, YER184C-protein, ygl096w-protein, ymr324c-protein and YOR044W-protein, in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of abscisic acid, cryptoxanthin, violaxanthin and/or zeaxanthin or a composition comprising abscisic acid, cryptoxanthin, violaxanthin, and/or zeaxanthin in said non-human organism or in the culture medium surrounding said non-human organism.

Item 2. A process for the production of a fine chemical selected from the group consisting of: abscisic acid, cryptoxanthin, violaxanthin and/or zeaxanthin, which comprises
(A)
   (i) increasing or generating of the expression of; and/or
   (ii) increasing or generating the expression of an expression product of; and/or
   (iii) increasing or generating one or more activities of an expression product encoded by;
      at least one respective nucleic acid molecule comprising a nucleic acid molecule selected from the group consisting of:
   (a) a nucleic acid molecule encoding the polypeptide shown in column 5 or 8 of Table II, preferably Table II B, application no.13, or a homolog or a fragment thereof;
   (b) a nucleic acid molecule shown in column 5 or 8 of Table I, preferably Table I B, application no.13, or a homolog or a fragment thereof (preferably the coding region thereof);
   (c) a nucleic acid molecule, which, as a result of the degeneracy of the genetic code, can be derived from a polypeptide sequence depicted in column 5 or 8 of Table II, preferably Table II B, application no.13;
   (d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 13, or the coding region thereof;
   (e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no.13;
   (i) a nucleic acid molecule encoding a polypeptide having the activity represented by a respective protein as depicted in column 5 of Table II, application no. 13;
   (j) a nucleic acid molecule which comprises a polynucleotide, which is obtained by amplifying a cDNA library or a genomic library using the primers as depicted in column 8 of Table III, application no. 13; and
   (k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
   or a nucleic acid molecule comprising a sequence which is complementary thereto;
   in a non-human organism or a part thereof, preferably a microorganism, a plant cell, a plant or a part thereof, as compared to a corresponding non-transformed wild type non-human organism or a part thereof; and
(B) growing the non-human organism or a part thereof under conditions which permit the production of abscisic acid, cryptoxanthin, violaxanthin and/or zeaxanthin or a composition comprising abscisic acid, cryptoxanthin, violaxanthin and/or zeaxanthin in said non-human organism or in the culture medium surrounding said non-human organism.

Item 3. A process of items 1 or 2, comprising of recovering abscisic acid, cryptoxanthin, violaxanthin and/or zeaxanthin in its free or bound form.

Item 4. The process of any one of items 1 to 3, comprising the folowing steps:
(a) selecting a non-human organism or a part thereof expressing a polypeptide encoded by the nucleic acid molecule characterized in item 2;
(b) mutagenizing the selected non-human organism or the part thereof;
(c) comparing the activity or the expression level of said polypeptide in the mutagenized non-human organism or the part thereof with the activity or the expression of said polypeptide of the selected non-human organisms or the part thereof;
(d) selecting the mutated non-human organisms or parts thereof, which comprise an increased activity or expression level of said polypeptide compared to the selected non-human organism or the part thereof;
(e) optionally, growing and cultivating the non-human organisms or the parts thereof;
and
(f) recovering, and optionally isolating, the free or bound abscisic acid, cryptoxanthin, violaxanthin and/or zeaxanthin produced by the selected mutated non-human organisms or parts thereof.

Item 5. The process of any one of items 1 to 4 wherein the activity of said protein or the expression of said nucleic acid molecule is increased or generated transiently or stably.

Item 6. An isolated nucleic acid molecule comprising a nucleic acid molecule selected from the group consisting of:
(a) a nucleic acid molecule encoding a polypeptide shown in Table II, application no. 13, column 5 or 8, preferably shown in Table II B, application no. 13, column 8, or a homolog or a fragment thereof;
(b) a nucleic acid molecule shown in Table I, application no. 13, column 5 or 8, preferably shown in Table I B, application no. 13, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 13, preferably in column 8 of Table II B, application no. 13;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 13, preferably shown in column 8 of Table I B, application no. 13, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 13;
(i) a nucleic acid molecule encoding a polypeptide having the activity represented by a respective protein as depicted in column 5 of Table II, application no. 13;
(j) a nucleic acid molecule comprising a polynucleotide which is obtained by amplifying a cDNA library or a genomic library using the primers in Table III, application no. 13, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (i) ,
or a nucleic acid molecule comprising a sequence which is complementary thereto.

Item 7. A nucleic acid construct which confers the expression of the nucleic acid molecule as defined in item 6, comprising one or more regulatory elements.

Item 8. An expression cassette comprising
(a) a promoter, preferably selected from the group consisting of Big35S, PCUbi, Super and USP;
(b) a nucleic acid molecule as defined in item 6.

Item 9. A vector comprising the nucleic acid molecule as defined in item 6 or the nucleic acid construct as claimed in item 7 or the expression cassette as claimed in item 8.

Item 10. A host cell or a plant organelle, which has been transformed with the vector as claimed in item 9 or the nucleic acid molecule as defined in item 6 or the nucleic acid construct as claimed in item 7 or the expression cassette as claimed in item 8.

Item 11. The host cell of item 10, which is a plant cell or a microorganism.

Item 12. A process for producing a polypeptide, wherein the polypeptide is expressed in a host cell as claimed in item 10 or 11.

Item 13. A polypeptide produced by the process as claimed in item 12 or encoded by the nucleic acid molecule as defined in item 6.

Item 14. An antibody, which binds specifically to the polypeptide as claimed in item 13.

Item 15. A plant tissue, propagation material, harvested material or a plant comprising the host cell as claimed in item 10 or 11.

Item 16. A process for the identification of a compound conferring an increase in abscisic acid, cryptoxanthin, violaxanthin and/or zeaxanthin production in a non-human organism, comprising the steps:
(a) culturing a plant cell or tissue or microorganism or maintaining a plant expressing the polypeptide encoded by the nucleic acid molecule as defined in item 6 conferring an increase in the amount of abscisic acid, cryptoxanthin, violaxanthin and/or zeaxanthin in a non-human organism or a part thereof and a readout system capable of interacting with the polypeptide under suitable conditions which permit the interaction of the polypeptide with said readout system in the presence of a compound or a sample comprising a plurality of compounds and capable of providing a detectable signal in response to the binding of a compound to said polypeptide under conditions which permit the expression of said readout system and of the polypeptide encoded by the nucleic acid molecule of item 6 conferring an increase in the amount of abscisic acid, cryptoxanthin, violaxanthin and/or zeaxanthinin a non-human organism or a part thereof;
(b) identifying if the compound is an effective agonist by detecting the presence or absence or increase of a signal produced by said readout system.

Item 17. A method for the production of an agricultural composition comprising the steps of the method of item 16 and formulating the compound identified in item 16 in a form acceptable for an application in agriculture.

Item 18. A composition comprising the nucleic acid molecule as defined in item 6, the polypeptide as claimed in item 13, the nucleic acid construct as claimed in item 7, the expression cassette as claimed in item 8, the vector as claimed in item 9, the compound as claimed in item 16, the antibody as claimed in item 14, and optionally an agricultural acceptable carrier.

Item 19. Use of the nucleic acid molecule as defined in item 6 for the identification of a nucleic acid molecule conferring an increase in abscisic acid, cryptoxanthin, violaxanthin and/or zeaxanthin after expression.

Item 20. Cosmetic, pharmaceutical, food or feed composition comprising the nucleic acid moleculeas defined in item 6, the polypeptide as claimed in item 13, the nucleic acid construct as claimed in item 7, the expression cassette as claimed in item 8, the vector as claimed in item 9, the antibody as claimed in item 14 , the plant or plant tissue as claimed in item 15, the harvested material as claimed in item 15 or the host cell of item as claimed in items 10 or 11.

Item 21. Use of the nuclei acid molecule as defined in item 6, the polypeptide as claimed in item 13, the nucleic acid construct as claimed in item 7, the expression cassette as claimed in item 8, the vector as claimed in item 9, the plant or plant tissue as claimed in item 15, or the host cell of item 10 to 11 for the production of plant resistant to a herbicide inhibiting the production of abscisic acid, cryptoxanthin, violaxanthin and/or zeaxanthin.

Item 22. A method for producing a plant with increased yield as compared to a corresponding non-transformed wild type plant, whereby the method comprises
(i) increasing or generating of the expression of; and/or
(ii) increasing or generating the expression of an expression product of; and/or
(iii) increasing or generating one or more activities of an expression product encoded by;
   at least one respective nucleic acid molecule comprising a nucleic acid molecule selected from the group consisting of:
(a) a nucleic acid molecule encoding the polypeptide shown in column 5 or 8 of Table II, preferably Table II B, application no. 13, or a homolog or a fragment thereof, in case in column 7 abscisic acid is indicated;
(b) a nucleic acid molecule shown in column 5 or 8 of Table I, preferably Table I B, application no. 13, or a homolog or a fragment thereof (preferably the coding region thereof) , in case in column 7 abscisic acid is indicated;
(c) a nucleic acid molecule, which, as a result of the degeneracy of the genetic code, can be derived from a polypeptide sequence depicted in column 5 or 8 of Table II, preferably Table II B, application no. 13, in case in column 7 abscisic acid is indicated;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 13, or the coding region thereof, in case in column 7 abscisic acid is indicated;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 13, in case in column 7 abscisic acid is indicated;
(i) a nucleic acid molecule encoding a polypeptide having the activity represented by a respective protein as depicted in column 5 of Table II, application no. 13, in case in column 7 abscisic acid is indicated;
(j) a nucleic acid molecule which comprises a polynucleotide, which is obtained by amplifying a cDNA library or a genomic library using the primers as depicted in column 8 of Table III, application no. 13, in case in column 7 abscisic acid is indicated; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto;
   in a plant cell, plant or a part thereof.

Item 23. A method for producing a plant with increased yield according to item 22, which comprises in addition the regeneration of a plant from such transformed plant cell, plant cell compartment, plant cell nucleus, plant cell or plant tissue with increased yield.

[0483.1.7.13] to [0492.1.7.13] for the disclosure of these paragraphs see [0483.1.7.7] to [0492.1.7.7] above

### Example 10

### Purification of the Xanthophylls and/or Abcisic Acid and Determination of the Carotenoids Content

### Abbreviations:

GC-MS, gas liquid chromatography/mass spectrometry; TLC, thin-layer chromatography. The unambiguous detection for the presence of xanthophylls can be obtained by analyzing recombinant organisms using analytical standard methods: LC, LC-MSMS or TLC, as described

The total xanthophylls produced in the organism for example in algae used in the inventive process can be analysed for example according to the following procedure:

The material such as algae or plants to be analyzed can be disrupted by sonication, grinding in a glass mill, liquid nitrogen and grinding or via other applicable methods.

Plant material is initially homogenized mechanically by comminuting in a pestle and mortar to make it more amenable to extraction. [0494.1.7.13] for the disclosure of this paragraph see [0494.1.7.7] above

### Example 11a

### PCR Amplification of the Sequences

Unless otherwise specified, standard methods as described in Sambrook et al., Molecular Cloning: A laboratory manual, Cold Spring Harbor 1989, Cold Spring Harbor Laboratory Press are used.

The inventive sequences as shown in the respective line in Table I, column 5, preferably the coding region thereof, were amplified by PCR as described in the protocol of the Pfu Ultra, Pfu Turbo or Herculase DNA polymerase (Stratagene). The composition for the protocol of the Pfu Ultra, Pfu Turbo or Herculase DNA polymerase was as follows: 1×PCR buffer (Stratagene), 0.2 mM of each dNTP, 100 ng genomic DNA of Saccharomyces cerevisiae (strain S288C; Research Genetics, Inc., now Invitrogen), Escherichia coli (strain MG1655; E. coli Genetic Stock Center), Synechocystis sp. (strain PCC6803), Azotobacter vinelandii (strain N.R. Smith,16), Thermus thermophilus (HB8) or 50 ng cDNA from various tissues and development stages of Arabidopsis thaliana (ecotype Columbia), Physcomitrella patens, Glycine max (variety Resnick), Brassica napus, Oryza sativa or Zea mays (variety B73, Mo17, A188), 50 pmol forward primer, 50 pmol reverse primer, with or without 1 M Betaine, 2.5 u Pfu Ultra, Pfu Turbo or Herculase DNA polymerase.

The amplification cycles were as follows:

1 cycle of 2-3 minutes at 94-95° C., then 25-36 cycles with 30-60 seconds at 94-95° C., 30-45 seconds at 50-60° C. and 210-480 seconds at 72° C., followed by 1 cycle of 5-10 minutes at 72° C., then 4-16° C.—preferably for Saccharomyces cerevisiae, Escherichia coli, Synechocystis sp., Azotobacter vinelandii, Thermus thermophilus.

In case of Arabidopsis thaliana, Brassica napus, Glycine max, Oryza sativa, Physcomitrella patens, Zea mays the amplification cycles were as follows:

1 cycle with 30 seconds at 94° C., 30 seconds at 61° C., 15 minutes at 72° C., then 2 cycles with 30 seconds at 94° C., 30 seconds at 60° C., 15 minutes at 72° C., then 3 cycles with 30 seconds at 94° C., 30 seconds at 59° C., 15 minutes at 72° C., then 4 cycles with 30 seconds at 94° C., 30 seconds at 58° C., 15 minutes at 72° C., then 25 cycles with 30 seconds at 94° C., 30 seconds at 57° C., 15 minutes at 72° C., then 1 cycle with 10 minutes at 72° C., then finally 4-16° C.

RNAs were generated with the RNeasy Plant Kit according to the standard protocol (Qiagen) and Superscript II Reverse Transkriptase was used to produce double stranded cDNA according to the standard protocol (Invitrogen).

ORF specific primer pairs for the genes to be expressed are shown in the respective line in Table III, column 8. The following adapter sequences were added to Saccharomyces cerevisiae ORF specific primers (see Table III) for cloning purposes:

SEQ ID NO: 20

| | | |
|---|---|---|
| i) | foward primer: | 5′-GGAATTCCAGCTGACCACC-3′ |

SEQ ID NO: 21

| | | |
|---|---|---|
| ii) | reverse primer: | 5′-GATCCCCGGGAATTGCCATG-3′ |

These adaptor sequences allow cloning of the ORF into the various vectors containing the Resgen adaptors, see table column E of Table c.

The following adapter sequences were added to Saccharomyces cerevisiae, Escherichia coli, Synechocystis sp., Azotobacter vinelandii, Thermus thermophilus, Arabidopsis thaliana, Brassica napus, Glycine max, Oryza sativa , Physcomitrella patens, or Zea mays ORF specific primers for cloning purposes:

| SEQ ID NO: 22 | | |
|---|---|---|
| iii) | forward primer: | 5′-TTGCTCTTCC-3′ |
| SEQ ID NO: 23 | | |
| iiii) | reverse primer: | 5′-TTGCTCTTCG-3′ |

The adaptor sequences allow cloning of the ORF into the various vectors containing the Colic adaptors, see table column E of Table c.

Therefore for amplification and cloning of Saccharomyces cerevisiae SEQ ID NO: 63167, a primer consisting of the adaptor sequence i) and the ORF specific sequence SEQ ID NO: 63259 and a second primer consisting of the adaptor sequence ii) and the ORF specific sequence SEQ ID NO: 63260 were used.

For amplification and cloning of Escherichia coli SEQ ID NO: 7941, a primer consisting of the adaptor sequence iii) and the ORF specific sequence SEQ ID NO: 7945 and a second primer consisting of the adaptor sequence iiii) and the ORF specific sequence SEQ ID NO: 7946 were used.

For amplification and cloning of Synechocystis sp. SEQ ID NO: 53189, a primer consisting of the adaptor sequence iii) and the ORF specific sequence SEQ ID NO: 53447 and a second primer consisting of the adaptor sequence iiii) and the ORF specific sequence SEQ ID NO: 53448 were used.

For amplification and cloning of Arabidopsis thaliana SEQ ID NO: 2573, a primer consisting of the adaptor sequence iii) and the ORF specific sequence SEQ ID NO: 2931 and a second primer consisting of the adaptor sequence iiii) and the ORF specific sequence SEQ ID NO: 2932 were used.

For amplification and cloning of Azotobacter vinelandii SEQ ID NO: 70715, a primer consisting of the adaptor sequence iii) and the ORF specific sequence SEQ ID NO: 71321 and a second primer consisting of the adaptor sequence iiii) and the ORF specific sequence SEQ ID NO: 71322 were used.

For amplification and cloning of Glycine max SEQ ID NO: 46751, a primer consisting of the adaptor sequence iii) and the ORF specific sequence SEQ ID NO: 46841 and a second primer consisting of the adaptor sequence iiii) and the ORF specific sequence SEQ ID NO: 46842 were used.

For amplification and cloning of Oryza sativa SEQ ID NO: 34301, a primer consisting of the adaptor sequence iii) and the ORF specific sequence SEQ ID NO: 34595 and a second primer consisting of the adaptor sequence iiii) and the ORF specific sequence SEQ ID NO: 34596were used.

Following these examples every sequence disclosed in Table I, preferably column 5, especially the coding region thereof can be cloned by fusing the adaptor sequences to the respective specific primers sequences as disclosed in Table III, column 8 using the respective vectors shown in Table c.

[0496.1.7.13] to [0499.1.7.13] for the disclosure of these paragraphs see [0496.1.7.7] to [0499.1.7.7] above

### Example 11e

### Cloning of Inventive Sequences as Shown in Table I, Column 5 in the Different Expression Vectors

For cloning of for example SEQ ID NO: 7941 from Escherichia coli or any other ORF from Escherichia coli, Synechocystis sp., Azotobacter vinelandii, Arabidopsis thaliana, Glycine max, or Oryza sativa the vector DNA was treated with the restriction enzymes Pacl and Ncol following the standard protocol (MBI Fermentas).

For cloning for example the ORFs of SEQ ID NO: 63168 from Saccharomyces cerevisiae or any other ORF from Saccharomyces cerevisiae into vectors containing the Resgen adaptor sequence the respective vector DNA was treated with the restriction enzyme Ncol.

For cloning of for example ORFs SEQ ID NO: 67647 from Saccharomyces cerevisiae or any other ORF from Saccharomyces cerevisiae into vectors containing the Colic adaptor sequence, the respective vector DNA was treated with the restriction enzymes Pacl and Ncol following the standard protocol (MBI Fermentas).

In all cases the reaction was stopped by inactivation at 70° C. for 20 minutes and purified over QlAquick or NucleoSpin Extract II columns following the standard protocol (Qiagen or Macherey-Nagel).

Then the PCR-product representing the amplified ORF with the respective adapter sequences and the vector DNA were treated with T4 DNA polymerase according to the standard protocol (MBI Fermentas) to produce single stranded overhangs with the parameters 1 unit T4 DNA polymerase at 37° C. for 2-10 minutes for the vector and 1-2 u T4 DNA polymerase at 15-17° C. for 10-60 minutes for the PCR product representing SEQ ID NO: 7941.

The reaction was stopped by addition of high-salt buffer and purified over QlAquick or N ucleoSpin Extract II columns following the standard protocol (Qiagen or Macherey-Nagel).

According to this example the skilled person is able to clone all sequences disclosed in Table I, preferably column 5 or column 8, especially the coding region thereof.

Approximately 30-60 ng of prepared vector and a defined amount of prepared amplificate were mixed and hybridized at 65° C. for 15 minutes followed by 37° C. 0.1° C/1 seconds, followed by 37° C. 10 minutes, followed by 0.1° C/1 seconds, then 4-10° C.

The ligated constructs were transformed in the same reaction vessel by addition of competent E. coli cells (strain DHSalpha) and incubation for 20 minutes at 1° C. followed by a heat shock for 90 seconds at 42° C. and cooling to 1-4° C. Then, complete medium (SOC) was added and the mixture was incubated for 45 minutes at 37° C. The entire mixture was subsequently plated onto an agar plate with 0.05 mg/ml kanamycin and incubated overnight at 37° C.

The outcome of the cloning step was verified by amplification with the aid of primers which bind upstream and downstream of the integration site, thus allowing the amplification of the insertion. The amplifications were carried out as described in the protocol of Taq DNA polymerase (Gibco-BRL).

The amplification cycles were as follows:

1 cycle of 1-5 minutes at 94° C., followed by 35 cycles of in each case 15-60 seconds at 94° C., 15-60 seconds at 50-66° C. and 5-15 minutes at 72° C., followed by 1 cycle of 10 minutes at 72° C., then 4-16° C.

Several colonies were checked, but only one colony for which a PCR product of the expected size was detected was used in the following steps.

A portion of this positive colony was transferred into a reaction vessel filled with complete medium (LB) supplemented with kanamycin and incubated overnight at 37° C.

The plasmid preparation was carried out as specified in the Qiaprep or NucleoSpin Multi-96 Plus standard protocol (Qiagen or Macherey-Nagel).

[0501.1.7.13] to [0503.1.7.13] for the disclosure of these paragraph see [0501.1.7.7] to [0503.1.7.7] above

Table d showing results of plant analyses

TABLE d

| SeqID | Target | Locus | Metabolite | Source | Promotor | Method | Min | Max |
|---|---|---|---|---|---|---|---|---|
| 130491 | non-targ | At3g15360 | cryptoxanthin | ARA_LEAF | p-PcUBI | LC | 36 | 59 |
| 120952 | non-targ | At3g16820 | zeaxanthin | ARA_LEAF | p-PcUBI | LC | 24 | 36 |
| 2573 | non-targ | At3g62930 | zeaxanthin | ARA_LEAF | p-PcUBI | LC | 23 | 57 |
| 23482 | non-targ | At4g15660 | zeaxanthin | ARA_LEAF | p-PcUBI | LC | 23 | 53 |
| 3279 | non-targ | At4g15670 | zeaxanthin | ARA_LEAF | p-PcUBI | LC | 26 | 60 |
| 68849 | non-targ | At4g26080 | zeaxanthin | ARA_LEAF | p-PcUBI | LC | 23 | 41 |
| 4102 | non-targ | At4g33040 | cryptoxanthin | ARA_LEAF | p-PcUBI | LC | 32 | 64 |
| 102899 | non-targ | At5g60440 | zeaxanthin | ARA_LEAF | p-PcUBI | LC | 25 | 46 |
| 102941 | non-targ | Avin- | zeaxanthin | ARA_LEAF | p-PcUBI | LC | 36 | 46 |
| | | DRAFT_4847 | | | | | | |
| 34301 | non-targ | AX653549 | cryptoxanthin | ARA_LEAF | p-PcUBI | LC | 34 | 52 |
| 7941 | non-targ | B1003 | zeaxanthin | ARA_LEAF | p-Super | LC | 26 | 51 |
| 36880 | non-targ | B1023 | zeaxanthin | ARA_LEAF | p-Super | LC | 27 | 46 |
| 8316 | non-targ | B1838 | zeaxanthin | ARA_LEAF | p-Super | LC | 23 | 47 |
| 8920 | non-targ | B2032 | zeaxanthin | ARA_LEAF | p-Super | LC | 27 | 59 |
| 115595 | non-targ | B2099 | cryptoxanthin | ARA_LEAF | p-Super | LC | 32 | 53 |
| 39013 | non-targ | B2121 | zeaxanthin | ARA_LEAF | p-Super | LC | 37 | 40 |
| 40329 | non-targ | B2474 | zeaxanthin | ARA_LEAF | p-Super | LC | 22 | 42 |
| 40741 | non-targ | B2714 | zeaxanthin | ARA_LEAF | p-Super | LC | 23 | 38 |
| 115719 | non-targ | B2790 | cryptoxanthin | ARA_LEAF | p-Super | LC | 29 | 43 |
| 41073 | non-targ | B2914 | violaxanthin | ARA_SEED_2 | p-USP | LC | 50 | 220 |
| 9492 | non-targ | B3256 | violaxanthin | ARA_SEED_2 | p-USP | LC | 36 | 220 |
| 10104 | non-targ | B3346 | cryptoxanthin | ARA_LEAF | p-Super | LC | 47 | 60 |
| 94386 | non-targ | B3774 | violaxanthin | ARA_SEED_2 | p-USP | LC | 35 | 232 |
| 10708 | non-targ | B3817 | cryptoxanthin | ARA_LEAF | p-Super | LC | 34 | 66 |
| 105659 | plastidic | B3908 | violaxanthin | ARA_SEED_2 | p-USP | LC | 47 | 66 |
| 44378 | non-targ | B4012 | violaxanthin | ARA_SEED_2 | p-USP | LC | 39 | 118 |
| 130673 | non-targ | B4120 | cryptoxanthin | ARA_LEAF | p-Super | LC | 37 | 74 |
| 130673 | non-targ | B4120 | zeaxanthin | ARA_LEAF | p-Super | LC | 25 | 30 |
| 45556 | non-targ | B4384 | violaxanthin | ARA_SEED_2 | p-USP | LC | 92 | 324 |
| 46751 | non-targ | GM02LC19289 | zeaxanthin | ARA_LEAF | p-PcUBI | LC | 33 | 36 |
| 53189 | non-targ | Sll1185 | cryptoxanthin | ARA_LEAF | p-PcUBI | LC | 35 | 47 |
| 53189 | non-targ | Sll1185 | zeaxanthin | ARA_LEAF | p-PcUBI | LC | 28 | 48 |
| 58823 | plastidic | Slr1755 | zeaxanthin | ARA_LEAF | p-PcUBI | LC | 27 | 46 |
| 63167 | non-targ | Ycl026c-a | cryptoxanthin | ARA_LEAF | Big35S | LC | 32 | 79 |
| 14706 | plastidic | Ydr131c | cryptoxanthin | ARA_LEAF | p-Super | LC | 38 | 133 |
| 90278 | plastidic | Ygl096w | cryptoxanthin | ARA_LEAF | p-Super | LC | 39 | 59 |
| 130742 | plastidic | Ygl256w | cryptoxanthin | ARA_LEAF | p-Super | LC | 35 | 57 |
| 130903 | non-targ | Ykr072c | cryptoxanthin | ARA_LEAF | Big35S | LC | 50 | 76 |
| 130903 | non-targ | Ykr072c | zeaxanthin | ARA_LEAF | Big35S | LC | 25 | 52 |
| 130932 | plastidic | Ymr281w | zeaxanthin | ARA_LEAF | p-Super | LC | 28 | 81 |
| 130986 | plastidic | Ymr324c | cryptoxanthin | ARA_LEAF | p-Super | LC | 31 | 49 |
| 67646 | plastidic | Ynr019w | zeaxanthin | ARA_LEAF | p-PcUBI | LC | 33 | 49 |
| 130713 | non-targ | Yer184c | abscisic acid | ARA_LEAF | Big35S | LC | 31 | 114 |
| 130999 | non-targ | YOR044W | abscisic acid | ARA_LEAF | Big35S | LC | 3 | 113 |
| 45394 | non-targ | B4321 | abscisic acid | ARA_LEAF | p-Super | LC | 25 | 283 |
| 54337 | mitochondrial | Sll1450 | abscisic acid | ARA_LEAF | p-PcUBI | LC | 22 | 115 |
| 86447 | plastidic | Sll1848 | abscisic acid | ARA_LEAF | p-PcUBI | LC | 26 | 364 |
| 68657 | non-targ | At3g02990 | abscisic acid | ARA_LEAF | p-PcUBI | LC | 14 | 231 |
| 18235 | non-targ | At1g48260 | abscisic acid | ARA_LEAF | p-PcUBI | LC | 29 | 300 |
| 19419 | non-targ | At1g72770 | abscisic acid | ARA_LEAF | p-PcUBI | LC | 124 | 1187 |
| 70715 | non-targ | AVIN- | abscisic acid | ARA_LEAF | p-PcUBI | LC | 12 | 162 |
| | | DRAFT_0539 | | | | | | |
| 20346 | non-targ | At2g30540 | abscisic acid | ARA_LEAF | p-PcUBI | LC | 11 | 152 |

Column 1 shows the SEQ ID NO, Column 2 shows the expression type (targeted or non-targeted), Column 3 shows the “gene name” (locus), Column 4 shows the metabolite analyzed, Column 5 indicates the A. thaliana source tissue analyzed, Column 6 indicates the used promoter for expression, Column 7 indicates the analytical method. Columns 8 and 9 show the minimum and the maximum increase of the analyzed metabolite (in percent) in comparison to the wild type (ratio_by_WT, given as percent increase).

The term “non-targ” in Table d, Column 2 which shows the expression type means “non-targeted”.

When the analyses were repeated independently, all results proved to be significant.

[0505.1.7.13] to [0515.1.7.13] for the disclosure of these paragraphs see [0505.1.7.7] to [0515.1.7.7] above

In a further embodiment, the present invention relates in paragraphs [0000.1.7.14.] to [0514.1.7.14.] to a further process for the production of the fine chemical betasitosterol, campesterol and/or stigmasterol as defined below and corresponding embodiments as described herein as follows.

for the disclosure of this paragraph see [0001.1.7.7] above

Sterols are a class of essential, natural compounds required by all eukaryotes to complete their life cycle. In animals, cholesterol is typically the major sterol while in fungi it is ergosterol. Plants produce a class of sterols called phytosterols. Phytosterols are natural components of many vegetables and grains. The term “phytosterols” covers plant sterols and plant stanols. Plant sterols are naturally occurring substances present in the diet as minor components of vegetable oils. The structures of these plant sterols are similar to that of cholesterol with an extra methyl or ethyl group and a double bond in the side chain. Saturated plant sterols, referred to as stanols, have no double bond in the ring structure.

Phytosterols (including plant sterols and stanols) are natural components of plant foods, especially plant oils, seeds and nuts, cereals and legumes specially of edible vegetable oils such as sunflower seed oil and, as such are natural constituents of the human diet. The most common phytosterols are Beta-sitosterol, campesterol, and stigmasterol. Beta-sitosterol is found in high amounts in nuts.

A high concentration of cholesterol in serum, i.e., hypercholesterolemia, is a wellknown risk factor for coronary heart disease (CH D). Blood cholesterol levels can be decreased by following diets, which are low in saturated fat, high in polyunsaturated fat and low in cholesterol. Although considerable achievements have been made in terms of knowledge and education, consumers still find it difficult to follow healthy eating advice. Both plant sterols and plant stanols (e.g. phytosterols) are effective in lowering plasma total and low density lipoprotein (LDL) cholesterol and this occurs by inhibiting the absorption of cholesterol from the small intestine. The plasma cholesterol-lowering properties of plant sterols have been known since the 1950s (Pollak, Circulation, 7, 702-706.1953). They have been used as cholesterol-lowering agents, first in a free form (Pollak and Kritchevsky, Sitosterol. In: Monographs on Aherosclerosis. Clarkson T B, Kritchevsky D,Pollak O J, eds. New York, Basel, Karger 1981; 1-219) and recently mainly as esterified phytosterols (Katan et al., Mayo Clin Proc 2003; 78: 965-978).

The consumption of plant sterols and plant stanols lowers blood cholesterol levels by inhibiting the absorption of dietary and endogenously-produced cholesterol from the small intestine and the plant sterols/stanols are only very poorly absorbed themselves. This inhibition is related to the similarity in physico-chemical properties of plant sterols and stanols and cholesterol. Plant sterols and plant stanols appear to be without hazard to health, having been shown without adverse effects in a large number of human studies. They show no evidence of toxicity even at high dose levels and gastro-intestinal absorption is low.

The most abundant sterols of vascular plants are campesterol, sitosterol and stigmasterol, all of which contain a double bond between the carbon atoms at positions 5 and 6 and are classified as delta-5 sterols.

Exemplary naturally occurring delta-5 plant sterols are isofucosterol, sitosterol, stigmasterol, campesterol, cholesterol, and dihydrobrassicasterol. Exemplary naturally occurring non-delta-5 plant sterols are cycloartenol, 24-methylene cycloartenol, cycloeucalenol, and obtusifoliol.

The ratio of delta-5 to non-delta-5 sterols in plants can be an important factor relating to insect pest resistance. Insect pests are unable to synthesize de novo the steroid nucleus and depend upon external sources of sterols in their food source for production of necessary steroid compounds. In particular, insect pests require an external source of delta-5 sterols. By way of example, externally provided delta-5 sterols are necessary for the production of ecdysteroids, hormones that control reproduction and development. See, e.g., Costet et al., Proc. Natl. Acad. Sci. USA, 84:643 (1987) and Corio-Costet et al., Archives of Insect Biochem. Physiol., 11:47 (1989).

The content of phytosterols in a cell was increased by increasing or decreasing certain activities as disclosed in WO2006/069610, WO2007/087815 or WO2008/034648. A detailled description of the state of the art can be found in WO2008/034648, paragraph [0005.1.13.13] (which is incorporated by reference).

The effectiveness of phytosterols as a dietary component to lower serum cholesterol level in humans has been well documented in the past years. Therefore, consumption of phytosterol-enriched food products by the general population and particularly mildly hypercholesterolemic subjects can be recommended. The FDA has approved the following claim for phytosterols: “Foods containing at least 0.4 gram per serving of plant sterols, eaten twice a day with meals for a daily total intake of at least 0.8 gram, as part of a diet low in saturated fat and cholesterol, may reduce the risk of heart disease.”

However, limited quantities of phytosterols are currently a major barrier in satisfying the demands for such functional foods. Transgenic plants having altered sterol profiles could be instrumental in establishing a dietary approach to cholesterol management and cardiovascular disease prevention. The altered phytosterol profile further leads to pest resistance.

Although people consume plant sterols every day in their normal diet, the amount is not great enough to have a significant blood cholesterol lowering effect. The intake of phytosterols varies among different populations according to the food products being consumed, but the average daily Western diet is reported to contain 150-300 mg of these sterols (de Vries et al., J Food Comp Anal 1997; 19: 115-141; Bjorkhem et al. Inborn errors in bile acid biosynthesis and storage of sterols other than cholesterol. In: The Metabolic and Molecular Bases of Inherited Disease. Scriver C S, Beaudet A L, Sly W S, Valle D, eds. New York, McGrawHill 2001; 2961-2988). In order to achieve a cholesterol-lowering benefit, approximately 1 g/day of plant sterols need to be consumed (Hendriks et al., European Journal of Clinical Nutrition, 53, 319-327.1999).

Phytosterols are found naturally in plant foods at low levels. The enrichment of foods such as margarines with plant sterols and stanols is one of the recent developments in functional foods to enhance the cholesterol-lowering ability of traditional food products. Incorporation of additional phytosterols into the diet may be an effective way of lowering total and LDL cholesterol levels. The non-esterified phytosterols can be used as novel food ingredients in: bakery products and cereals (eg, breakfast cereals, breakfast bars); dairy products such as low and reduced fat liquid milk, low and reduced fat yoghurt and yoghurt products, and dairy based desserts; non-carbonated soft drinks like low and reduced fat soy beverages and low and reduced fat soy-based yoghurts; meat products or edible fats and oils (eg, mayonnaise, spice sauces, salad dressings); margarine; and table spreads or dietary fats. [0009.1.14.14] When edible oils undergo normal refining, plant sterols are partially extracted. It is estimated that 2500 tonnes of vegetable oil needs to be refined to produce 1 tonne of plant sterols. Plant stanols are obtained by hydrogenation of the plant sterols.

Another source of plant sterols is tall oil, derived from the process of paper production from wood and approximately 2500 tons of pine is required to produce 1 ton of plant sterols. Tall oil also contains a higher proportion of plant stanols (primarily beta-sitostanol) than do vegetable oils.

The allowance by the FDA of a health claim for phytosterols and phytostanols has created much consumer interest and demand. There is a need to find new sources of phytosterols.

An economical method for producing phytosterols or its precursor and food- and feedstuffs with increased phytosterol content is therefore very important.

Particularly economical methods are biotechnological methods utilizing phytosterol-producing organisms, which are either natural or optimized by genetic modification.

There is a constant need for providing novel enzyme activities or direct or indirect regulators and thus alternative methods with advantageous properties for producing phytosterols or its precursor in organisms, e.g. in transgenic organisms.

To ensure a high quality of foods and animal feeds, it is therefore necessary to replace cholesterin by phytosterols. Accordingly, there is still a great demand for new and more suitable genes, which encode enzymes or regulators, which participate in the biosynthesis of phytosterols and make it possible to produce certain phytosterols specifically on an industrial scale without unwanted byproducts being formed. In the selection of genes for biosynthesis or regulation two characteristics above all are particularly important. On the one hand, there is as ever a need for improved processes for obtaining the highest possible contents of phytosterols and on the other hand as less as possible byproducts should be produced in the production process.

There is a demand especially for plants producing phytosterols in much greater quantities, preferably as compared to a control or wild type plant, where the phytosterols are preferably easy accessible and recoverable on an industrial scale without unwanted byproducts forming.

It is an object of the present invention to develop an inexpensive process for the synthesis of beta-sitosterol, campesterol, and/or stigmasterol.

It is a further object of the present invention to develop an inexpensive process for the synthesis of phytosterols, preferably beta-sitosterol, campesterol, and/or stigmasterol and to assure that the phytosterols, preferably beta-sitosterol, campesterol, and/or stigmasterol are more accessible and facilely to isolate and recover in an industrial scale from the producing organism, preferably from a plant.

for the disclosure of this paragraph see [0013.1.7.7] above

Accordingly, in a first embodiment, the invention relates to a process for the production of at least one fine chemical selected from the group consisting of: beta-sitosterol, campesterol, and stigmasterol, or, in other words, of the “fine chemical” or “fine chemical of the invention”.

The terms “fine chemical of the invention”, “fine chemical” or “the fine chemical” are used herein equally and relate in context of the paragraphs or sections [0014.1.14.14] to [0514.1.7.14] essentially to the metabolite or the metabolites indicated in column 7, application no. 14 of Tables I, II and IV in the respective line.

Further, the term “in context of any of the paragraphs [0014.1.14.14] to [0514.1.7.14]” as used herein means that for any of said paragraphs [0014.1.14.14] to [0514.1.7.14] the term “the fine chemical” is understood to follow the definition of paragraphs or sections [0014.1.14.14] and [0015.1.14.14], independently whether it refers to any other paragraph or not and whether the reference recites the term “fine chemical” in an other context.

Thus, in cases where one or more paragraphs or sections are incorporated by reference into any of the present paragraphs [0014.1.14.14] to [0514.1.7.14], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to the definition of paragraph [0014.1.14.14] and [0015.1.14.14].

Accordingly, in one embodiment, the term “fine chemical of the invention”, “fine chemical” or “the fine chemical” as used herein (alone or in combination, like in FCRP) means “betasitosterol in context of the nucleic acid or polypeptide sequences listed in the respective same line of any one of Tables Ito IV of application no. 14 and indicating in column 7 the metabolite “beta-sitosterol”.

In one embodiment, the term beta-sitosterol or the term “fine chemical” mean in context of the paragraphs or sections [0014.1.14.14] to [0514.1.7.14] at least one chemical compound with an activity of the above mentioned beta-sitosterol, respectively.

Accordingly, in one embodiment, the term “fine chemical of the invention”, “fine chemical” or “the fine chemical” as used herein (alone or in combination, like in FCRP) means “campesterol in context of the nucleic acid or polypeptide sequences listed in the respective same line of any one of Tables Ito IV of application no. 14 and indicating in column 7 the metabolite “campesterol”.

In one embodiment, the term campesterol or the term “fine chemical” mean in context of the paragraphs or sections [0014.1.14.14] to [0514.1.7.14] at least one chemical compound with an activity of the above mentioned campesterol, respectively.

Accordingly, in one embodiment, the term “fine chemical of the invention”, “fine chemical” or “the fine chemical” as used herein (alone or in combination, like in FCRP) means “stigmasterol in context of the nucleic acid or polypeptide sequences listed in the respective same line of any one of Tables I to IV of application no. 14 and indicating in column 7 the metabolite “stigmasterol”.

In one embodiment, the term stigmasterol or the term “fine chemical” mean in context of the paragraphs or sections [0014.1.14.14] to [0514.1.7.14] at least one chemical compound with an activity of the above mentioned stigmasterol, respectively.

Accordingly, in one embodiment, the terms “fine chemical of the invention”, “fine chemical” or “the fine chemical” mean in context of any of the paragraphs [0014.1.14.14] to [0514.1.7.14] beta-sitosterol, its salts, ester or ether in free form or bound form, for example acylated (beta-sitosterol esters), alkylated (beta-sitosteryl alkyl ethers), sulfated (beta-sitosterol sulfate), or linked to a glycoside moiety (beta-sitosteryl glycosides) which can be itself acylated (acylated sterol glycosides).

In a preferred embodiment, the term “the fine chemical” means beta-sitosterol or its salts, ester or ether, in free form or bound form.

In a preferred embodiment “beta-sitosterol” means beta-sitosterol in free form.

Accordingly, in one embodiment, the terms “fine chemical of the invention”, “fine chemical” or “the fine chemical” mean in context of any of the paragraphs [0014.1.14.14] to [0514.1.7.14] campesterol, its salts, ester or ether in free form or bound form, for example acylated (campesterol esters), alkylated (campesteryl alkyl ethers), sulfated (campesterol sulfate), or linked to a glycoside moiety (campesteryl glycosides) which can be itself acylated (acylated sterol glycosides). In a preferred embodiment, the term “the fine chemical” means campesterol or its salts, ester or ether, in free form or bound form.

In a preferred embodiment “campesterol” means campesterol in free form.

Accordingly, in one embodiment, the terms “fine chemical of the invention”, “fine chemical” or “the fine chemical” mean in context of any of the paragraphs [0014.1.14.14] to [0514.1.7.14] stigmasterol, its salts, ester or ether in free form or bound form. In a preferred embodiment, the term “the fine chemical” means stigmasterol or its salts, ester or ether, in free form or bound form, for example acylated (stigmasterol esters), alkylated (stigmasteryl alkyl ethers), sulfated (stigmasterol sulfate), or linked to a glycoside moiety (stigmasteryl glycosides) which can be itself acylated (acylated sterol glycosides).

In a preferred embodiment “stigmasterol” means stigmasterol in free form.

Further, the term “in context of any of the paragraphs [0014.1.14.14] to [0514.1.7.14]” as used herein means that for any of said paragraphs [0014.1.14.14] to [0514.1.7.14] the term “the fine chemical” is understood to follow the definition of section [0014.1.14.14] or section [0015.1.14.14], independently whether it refers to any other paragraph or not and whether the reference recites the term “fine chemical” in an other context.

Thus, in cases where one or more paragraphs or sections are incorporated by reference into any of the present paragraphs [0014.1.14.14] to [0514.1.7.14], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to this definition of this paragraph [0015.1.14.14].

Further, the term “fine chemicals” as used herein relates to compositions comprising said fine chemical(s), i.e. comprising beta-sitosterol, campesterol, and/or stigmasterol, respectively.

Accordingly, the present invention relates to a process for the production of campesterol, which comprises
(a) increasing or generating one or more activities selected from the group consisting of C-4 sterol methyl oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of eukaryotic translation initiation factor 5 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of ankyrin repeat family protein in a non-human organism or a part thereof, preferably a microorganism organism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of sterol desaturase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of beta-sitosterol, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of beta-sitosterol, which comprises
(a) increasing or generating one or more activities selected from the group consisting of sec-independent protein translocase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating one or more activities selected from the group consisting of 2-oxoglutarate dehydrogenase E1 subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating one or more activities selected from the group consisting of CTP synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating one or more activities selected from the group consisting of CTP synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of recombinase A in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating one or more activities selected from the group consisting of hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating one or more activities selected from the group consisting of hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of threonine synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of L-ribulose-5-phosphate 4-epimerase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of glucose dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating one or more activities selected from the group consisting of ATP-binding component of a transport system in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating one or more activities selected from the group consisting of ATP-binding component of a transport system in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of membrane transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of putrescine transport system permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating one or more activities selected from the group consisting of multidrug resistance protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of beta-sitosterol, which comprises
(a) increasing or generating one or more activities selected from the group consisting of sodium/proton antiporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating one or more activities selected from the group consisting of sodium/proton antiporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating one or more activities selected from the group consisting of phenylacetic acid degradation protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating one or more activities selected from the group consisting of phenylacetic acid degradation protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of b1401-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of lipoprotein precursor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating one or more activities selected from the group consisting of acid shock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating one or more activities selected from the group consisting of acid shock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of B1795-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of b2107-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of NADH dehydrogenase I chain N in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of amidophosphoribosyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of b2376-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of b2399-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of b2474-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of b2513-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating one or more activities selected from the group consisting of putative transport system permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating one or more activities selected from the group consisting of putative transport system permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of b2654-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of murein transglycosylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of xanthine dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of b3151-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating one or more activities selected from the group consisting of methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating one or more activities selected from the group consisting of threonine dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating one or more activities selected from the group consisting of b3989-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating one or more activities selected from the group consisting of b3989-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating one or more activities selected from the group consisting of b4029-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of stigmasterol, which comprises
(a) increasing or generating one or more activities selected from the group consisting of b4029-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stigmasterol or a composition comprising stigmasterol in said non-human organism or in the culture medium surrounding said non-human organism.

Accordingly, the present invention relates to a process for the production of beta-sitosterol, which comprises
(a) increasing or generating one or more activities selected from the group consisting of b4205-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of beta-sitosterol, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of sll0406-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of cation transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating one or more activities selected from the group consisting of CTP synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating one or more activities selected from the group consisting of CTP synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating one or more activities selected from the group consisting of phosphoribulokinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of beta-sitosterol, which comprises
(a) increasing or generating one or more activities selected from the group consisting of ABC metal ion transporter substrate-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating one or more activities selected from the group consisting of sll1761-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating one or more activities selected from the group consisting of acyl transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating one or more activities selected from the group consisting of cation-transporting ATPase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating one or more activities selected from the group consisting of permease protein of ABC transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of tryptophan synthase alpha chain in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating one or more activities selected from the group consisting of multiple antibiotic resistance protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating one or more activities selected from the group consisting of multiple antibiotic resistance protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating one or more activities selected from the group consisting of TTC1386-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating one or more activities selected from the group consisting of TTC1386-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of homocitrate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of homocitrate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of cyclin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of ydl235c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of polygalacturonase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating one or more activities selected from the group consisting of ynl321w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating one or more activities selected from the group consisting of acetyl-CoA acetyltransferase in a non-human organism or a part thereof, preferably a microorganism organism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of beta-sitosterol, which comprises
(a) increasing or generating one or more activities selected from the group consisting of ypr170c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating one or more activities selected from the group consisting of glucose-6-phosphate 1-dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

An embodiment of the present invention relates to a process for the production of beta-sitosterol, which comprises
(a1) increasing or generating the activity of a polypeptide comprising a polypeptide as depicted in a respective line in column 5 or 8 of Table II, application no. 14, or a homolog or a fragment thereof, a consensus sequence or at least one polypeptide motif as depicted in the respective line in column 8 of Table IV, application no. 14, whereby the respective line disclose in column 7 the fine chemical beta-sitosterol; or
(a2) increasing or generating the activity of an expression product of one or more nucleic acid molecule(s) comprising a polynucleotide as depicted in the respective line in column 5 or 8 of Table I, application no. 14, preferably the coding region thereof, or a homolog or a fragment thereof, whereby the respective line disclose in column 7 the fine chemical beta-sitosterol; or
(a3) increasing or generating the activity of a functional equivalent of (a1) or (a2); in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of beta-sitosterol or a composition comprising beta-sitosterol in said non-human organism or in the culture medium surrounding said non-human organism.

An embodiment of the present invention relates to a process for the production of campesterol, which comprises
(a1) increasing or generating the activity of a polypeptide comprising a polypeptide as depicted in a respective line in column 5 or 8 of Table II, application no. 14, or a homolog or a fragment thereof, a consensus sequence or at least one polypeptide motif as depicted in the respective line in column 8 of Table IV, application no. 14, whereby the respective line disclose in column 7 the fine chemical campesterol; or
(a2) increasing or generating the activity of an expression product of one or more nucleic acid molecule(s) comprising a polynucleotide as depicted in the respective line in column 5 or 8 of Table I, application no. 14, preferably the coding region thereof, or a homolog or a fragment thereof, whereby the respective line disclose in column 7 the fine chemical campesterol; or
(a3) increasing or generating the activity of a functional equivalent of (a1) or (a2); in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of campesterol or a composition comprising campesterol in said non-human organism or in the culture medium surrounding said non-human organism.

An embodiment of the present invention relates to a process for the production of stigmasterol, which comprises
(a1) increasing or generating the activity of a polypeptide comprising a polypeptide as depicted in a respective line in column 5 or 8 of Table II, application no. 14, or a homolog or a fragment thereof, a consensus sequence or at least one polypeptide motif as depicted in the respective line in column 8 of Table IV, application no. 14, whereby the respective line disclose in column 7 the fine chemical stigmasterol; or
(a2) increasing or generating the activity of an expression product of one or more nucleic acid molecule(s) comprising a polynucleotide as depicted in the respective line in column 5 or 8 of Table I, application no. 14, preferably the coding region thereof, or a homolog or a fragment thereof, whereby the respective line disclose in column 7 the fine chemical stigmasterol; or
(a3) increasing or generating the activity of a functional equivalent of (a1) or (a2); in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of stigmasterol or a composition comprising stigmasterol in said non-human organism or in the culture medium surrounding said non-human organism.

Accordingly, the increase or generation of one or more said activities is for example conferred by one or more expression products of said nucleic acid molecule, e.g. proteins. Accordingly, in the present invention described above, the increase or generation of one or more said activities is for example conferred by one or more protein(s) each comprising a polypeptide selected from the group as depicted in Table II, application no. 14, column 5 and 8, or a homolog or a fragment thereof.

The process of the invention comprises in one embodiment the following steps:
(i) increasing or generating of the expression of; and/or
(ii) increasing or generating the expression of an expression product of; and/or
(iii) increasing or generating one or more activities of an expression product encoded by;
   at least one nucleic acid molecule (in the following “Fine Chemical Related Protein (FCRP)”-encoding gene or “FCRP”-gene) comprising a nucleic acid molecule selected from the group consisting of:
(a) a nucleic acid molecule encoding the polypeptide shown in column 5 or 8 of Table II, preferably Table II B, application no. 14, or a homolog or a fragment thereof;
(b) a nucleic acid molecule shown in column 5 or 8 of Table I, preferably Table I B, application no. 14, or a homolog or a fragment thereof (preferably the coding region thereof);
(c) a nucleic acid molecule, which, as a result of the degeneracy of the genetic code, can be derived from a polypeptide sequence depicted in column 5 or 8 of Table II, preferably Table II B, application no. 14;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 14, or the coding region thereof;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d),
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 14;
(i) a nucleic acid molecule encoding a polypeptide having the activity represented by a respective protein as depicted in column 5 of Table II, application no. 14;
(j) a nucleic acid molecule which comprises a polynucleotide, which is obtained by amplifying a cDNA library or a genomic library using the primers as depicted in column 8 of Table III, application no. 14; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment thereof said nucleic acid molecule encodes a polypeptide which has the activity of the polypeptide represented by a protein comprising a polypeptide as depicted in the corresponding hit in column 5 of Table II, application no. 14.

In another preferred embodiment thereof said nucleic acid molecule confers a generation or an increase of the respective fine chemical in a non-human organism or a part thereof as compared to a corresponding wild-type non-human organism or part thereof.

In another preferred embodiment thereof said nucleic acid molecule encodes a polypeptide which has the activity of the polypeptide represented by a protein as depicted in the corresponding hit in column 5 of Table II, application no. 14, and confers a generation or an increase of the respective fine chemical in a non-human organism or a part thereof as compared to a corresponding wild-type non-human organism or part thereof.

Accordingly, the genes of the present invention or used in accordance with the present invention, which respectively encode a protein having an activity of 2-oxoglutarate dehydrogenase E1 subunit, ABC metal ion transporter substrate-binding protein, acetyl-CoA acetyltransferase, acetyltransferase, acid shock protein, acyl transferase, amidophosphoribosyltransferase, ankyrin repeat family protein, ATP-binding component of a transport system, b1401-protein, B1795-protein, b2107-protein, b2376-protein, b2399-protein, b2474-protein, b2513-protein, b2654-protein, b3151-protein, b3989-protein, b4029-protein, b4205-protein, C-4 sterol methyl oxidase, calcium-dependent protein kinase, cation transport protein, cationtransporting ATPase, CTP synthase, CTP synthetase, cyclin, cyclin D, eukaryotic translation initiation factor 5, glucose dehydrogenase, glucose-6-phosphate 1-dehydrogenase, glutaredoxin, homocitrate synthase, hydrolase, lipoprotein precursor, L-ribulose-5-phosphate 4-epimerase, membrane transport protein, methyltransferase, monothiol glutaredoxin, multidrug resistance protein, multiple antibiotic resistance protein, murein transglycosylase, NADH dehydrogenase I chain N, oxidoreductase, permease protein, permease protein of ABC transporter, phenylacetic acid degradation protein, phosphoribulokinase, polygalacturonase, protein kinase, protein phosphatase, putative transport system permease protein, putrescine transport system permease protein, recombinase A, sec-independent protein translocase, sll0406-protein, sll1761-protein, sodium/proton antiporter, sterol desaturase, threonine dehydrogenase, threonine synthase, transcription factor, tryptophan synthase alpha chain, TTC1386-protein, xanthine dehydrogenase, ydl235c-protein, ynl321w-protein, or ypr170c-protein, which respectively encode a protein comprising a polypeptide encoded by a nucleic acid sequence as shown in Table I, application no. 14, column 5 or 8, (preferably the coding region thereof), or a homolog or a fragment thereof, which respectively encode a protein comprising a polypeptide as depicted in Table II, application no. 14, column 5 or 8, or a homolg or a fragment thereof, and/or which respectively can be amplified with the primer set shown in Table III, application no. 14, column 8, are also referred to as “FCRP genes”.

Proteins or polypeptides encoded by “FCRP-genes” are referred to as “Fine Chemical Related Proteins” or “FCRP”. For the purposes of the description of the present invention, the respective protein having an activity of 2-oxoglutarate dehydrogenase E1subunit, ABC metal ion transporter substrate-binding protein, acetyl-CoA acetyltransferase, acetyltransferase, acid shock protein, acyl transferase, amidophosphoribosyltransferase, ankyrin repeat family protein, ATP-binding component of a transport system, b1401-protein, B1795-protein, b2107-protein, b2376-protein, b2399-protein, b2474-protein, b2513-protein, b2654-protein, b3151-protein, b3989-protein, b4029-protein, b4205-protein, C-4 sterol methyl oxidase, calcium-dependent protein kinase, cation transport protein, cation-transporting ATPase, CTP synthase, CTP synthetase, cyclin, cyclin D, eukaryotic translation initiation factor 5, glucose dehydrogenase, glucose-6-phosphate 1-dehydrogenase, glutaredoxin, homocitrate synthase, hydrolase, lipoprotein precursor, L-ribulose-5-phosphate 4-epimerase, membrane transport protein, methyltransferase, monothiol glutaredoxin, multidrug resistance protein, multiple antibiotic resistance protein, murein transglycosylase, NADH dehydrogenasel chain N, oxidoreductase, permease protein, permease protein of ABC transporter, phenylacetic acid degradation protein, phosphoribulokinase, polygalacturonase, protein kinase, protein phosphatase, putative transport system permease protein, putrescine transport system permease protein, recombinase A, sec-independent protein translocase, sll0406-protein, sll1761-protein, sodium/proton antiporter, sterol desaturase, threonine dehydrogenase, threonine synthase, transcription factor, tryptophan synthase alpha chain, TTC1386-protein, xanthine dehydrogenase, ydl235c-protein, ynl321w-protein, or ypr170c-protein, the respective protein comprising a polypeptide encoded by one or more respective nucleic acid sequences as shown in Table I, application no. 14, column 5 or 8, (preferably the coding region thereof), or a homolog or fragment thereof, the respective protein comprising a respective polypeptide as depicted in Table II, application no. 14, column 5 or 8, or a homolog or fragment thereof, the respective protein comprising a sequence corresponding to the consensus sequence as shown in Table IV, application no. 14, column 8, and/or the respective protein comprising at least one polypeptide motif as shown in Table IV, application no. 14, column 8 is also referred to as Fine Chemical Related Protein” or “FCRP”.

Thus, in one embodiment, the present invention provides a process of the production of beta-sitosterol, campesterol, and/or stigmasterol, by increasing or generating one or more activities, especially selected from the group consisting of 2-oxoglutarate dehydrogenase E1subunit, ABC metal ion transporter substrate-binding protein, acetyl-CoA acetyltransferase, acetyltransferase, acid shock protein, acyl transferase, amidophosphoribosyltransferase, ankyrin repeat family protein, ATP-binding component of a transport system, b1401-protein, B1795-protein, b2107-protein, b2376-protein, b2399-protein, b2474-protein, b2513-protein, b2654-protein, b3151-protein, b3989-protein, b4029-protein, b4205-protein, C-4 sterol methyl oxidase, calcium-dependent protein kinase, cation transport protein, cation-transporting ATPase, CTP synthase, CTP synthetase, cyclin, cyclin D, eukaryotic translation initiation factor 5, glucose dehydrogenase, glucose-6-phosphate 1-dehydrogenase, glutaredoxin, homocitrate synthase, hydrolase, lipoprotein precursor, L-ribulose-5-phosphate 4-epimerase, membrane transport protein, methyltransferase, monothiol glutaredoxin, multidrug resistance protein, multiple antibiotic resistance protein, murein transglycosylase, NADH dehydrogenase I chain N, oxidoreductase, permease protein, permease protein of ABC transporter, phenylacetic acid degradation protein, phosphoribulokinase, polygalacturonase, protein kinase, protein phosphatase, putative transport system permease protein, putrescine transport system permease protein, recombinase A, sec-independent protein translocase, sll0406-protein, sll1761-protein, sodium/proton antiporter, sterol desaturase, threonine dehydrogenase, threonine synthase, transcription factor, tryptophan synthase alpha chain, TTC1386-protein, xanthine dehydrogenase, ydl235c-protein, ynl321w-protein, and ypr170c-protein, which is conferred by one or more FCRPs or the gene product of one or more FCRP-genes, for example by the gene product of a nucleic acid sequences comprising a polynucleotide selected from the group as shown in Table 1, application no. 14, column 5 or 8, (preferably by the coding region thereof), or a homolog or a fragment thereof, e.g. or by one or more proteins each comprising a polypeptide encoded by one or more nucleic acid sequences selected from the group as shown in Table I, application no. 14, column 5 or 8, (preferably by the coding region thereof), or a homolog or a fragment thereof, or by one or more protein(s) each comprising a polypeptide selected from the group as depicted in Table II, application no. 14, column 5 and 8, or a homolog thereof, or a protein comprising a sequence corresponding to the consensus sequence or comprising at least one polypeptide motif as shown in Table IV, application no. 14, column 8.

for the disclosure of this paragraph see [0025.1.7.7] above

In an embodiment, the process comprises increasing or generating the activity of one or more polypeptides having said activity, e.g. by generating or increasing the amount and/or specific activity in the cell or a compartment of a cell of one of more FCRP, especially selected from the group consisting of 2-oxoglutarate dehydrogenase E1subunit, ABC metal ion transporter substrate-binding protein, acetyl-CoA acetyltransferase, acetyltransferase, acid shock protein, acyl transferase, amidophosphoribosyltransferase, ankyrin repeat family protein, ATP-binding component of a transport system, b1401-protein, B1795-protein, b2107-protein, b2376-protein, b2399-protein, b2474-protein, b2513-protein, b2654-protein, b3151-protein, b3989-protein, b4029-protein, b4205-protein, C-4 sterol methyl oxidase, calcium-dependent protein kinase, cation transport protein, cation-transporting ATPase, CTP synthase, CTP synthetase, cyclin, cyclin D, eukaryotic translation initiation factor 5, glucose dehydrogenase, glucose-6-phosphate 1-dehydrogenase, glutaredoxin, homocitrate synthase, hydrolase, lipoprotein precursor, L-ribulose-5-phosphate 4-epimerase, membrane transport protein, methyltransferase, monothiol glutaredoxin, multidrug resistance protein, multiple antibiotic resistance protein, murein transglycosylase, NADH dehydrogenase I chain N, oxidoreductase, permease protein, permease protein of ABC transporter, phenylacetic acid degradation protein, phosphoribulokinase, polygalacturonase, protein kinase, protein phosphatase, putative transport system permease protein, putrescine transport system permease protein, recombinase A, sec-independent protein translocase, sll0406-protein, sll1761-protein, sodium/proton antiporter, sterol desaturase, threonine dehydrogenase, threonine synthase, transcription factor, tryptophan synthase alpha chain, TTC1386-protein, xanthine dehydrogenase, ydl235c-protein, ynl321w-protein, and ypr170c-protein, for example of the respective polypeptide as depicted in Table II, application no. 14, column 5 and 8, or a homolog or a fragment thereof, or the respective polypeptide comprising a sequence corresponding to the consensus sequences as shown in Table IV, application no. 14, column 8, or the respective polypeptide comprising at least one polypeptide motif as depicted in Table IV, application no. 14, column 8.

A further embodiment of the present invention relates to a process for the production of campesterol, which comprises
(a) increasing or generating the activity of a C-4 sterol methyl oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a eukaryotic translation initiation factor 5 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a ankyrin repeat family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a sterol desaturase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of beta-sitosterol, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of beta-sitosterol, which comprises
(a) increasing or generating the activity of a sec-independent protein translocase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating the activity of a 2-oxoglutarate dehydrogenase E1 subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating the activity of a CTP synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating the activity of a CTP synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a recombinase A non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating the activity of a hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating the activity of a hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a threonine synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a L-ribulose-5-phosphate 4-epimerase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a glucose dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating the activity of a ATP-binding component of a transport system non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating the activity of a ATP-binding component of a transport system non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a membrane transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a putrescine transport system permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating the activity of a multidrug resistance protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of beta-sitosterol, which comprises
(a) increasing or generating the activity of a sodium/proton antiporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating the activity of a sodium/proton antiporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating the activity of a phenylacetic acid degradation protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating the activity of a phenylacetic acid degradation protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a b1401-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a lipoprotein precursor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating the activity of a acid shock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating the activity of a acid shock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a B1795-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a b2107-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a NADH dehydrogenase I chain N non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a amidophosphoribosyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a b2376-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a b2399-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a b2474-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a b2513-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating the activity of a putative transport system permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating the activity of a putative transport system permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a b2654-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a murein transglycosylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a xanthine dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a b3151-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating the activity of a methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating the activity of a threonine dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating the activity of a b3989-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating the activity of a b3989-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating the activity of a b4029-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of stigmasterol, which comprises
(a) increasing or generating the activity of a b4029-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stigmasterol or a composition comprising stigmasterol in said non-human organism or in the culture medium surrounding said non-human organism.

A further embodiment of the present invention relates to a process for the production of beta-sitosterol, which comprises
(a) increasing or generating the activity of a b4205-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of beta-sitosterol, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating the activity of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating the activity of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a sll0406-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a cation transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating the activity of a CTP synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating the activity of a CTP synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating the activity of a phosphoribulokinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of beta-sitosterol, which comprises
(a) increasing or generating the activity of a ABC metal ion transporter substrate-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating the activity of a sll1761-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating the activity of a acyl transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating the activity of a cation-transporting ATPase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating the activity of a permease protein of ABC transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a tryptophan synthase alpha chain non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating the activity of a multiple antibiotic resistance protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating the activity of a multiple antibiotic resistance protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating the activity of a TTC1386-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating the activity of a TTC1386-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a homocitrate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a homocitrate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a cyclin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a ydl235c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a polygalacturonase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating the activity of a ynl321w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating the activity of a acetyl-CoA acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of beta-sitosterol, which comprises
(a) increasing or generating the activity of a ypr170c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating the activity of a glucose-6-phosphate 1-dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating the activity of a polypeptide comprising a polypeptide as depicted in the respective line in column 5 or 8 of Table II, application no. 14, or a homolog or a fragment thereof, a consensus sequence or at least one polypeptide motif as depicted in the respective line in column 8 of Table IV, application no. 14, whereby the respective line disclose in column 7 the fine chemical beta-sitosterol; or
increasing or generating the activity of an expression product of one or more nucleic acid molecule(s) comprising a polynucleotide as depicted in the respective line in column 5 or 8 of Table I, application no. 14, preferably the coding region thereof, or a homolog or a fragment thereof, whereby the respective line disclose in column 7 the fine chemical beta-sitosterol;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of beta-sitosterol, or a composition comprising beta-sitosterol in said non-human organism or in the culture medium surrounding said non-human organism.

A further embodiment of the present invention relates to a process for the production of campesterol, which comprises
(a) increasing or generating the activity of a polypeptide comprising a polypeptide as depicted in the respective line in column 5 or 8 of Table II, application no. 14, or a homolog or a fragment thereof, a consensus sequence or at least one polypeptide motif as depicted in the respective line in column 8 of Table IV, application no. 14, whereby the respective line disclose in column 7 the fine chemical campesterol; or
increasing or generating the activity of an expression product of one or more nucleic acid molecule(s) comprising a polynucleotide as depicted in the respective line in column 5 or 8 of Table I, application no. 14, preferably the coding region thereof, or a homolog or a fragment thereof, whereby the respective line disclose in column 7 the fine chemical campesterol;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of campesterol, or a composition comprising campesterol in said non-human organism or in the culture medium surrounding said non-human organism.

A further embodiment of the present invention relates to a process for the production of stigmasterol, which comprises
(a) increasing or generating the activity of a polypeptide comprising a polypeptide as depicted in the respective line in column 5 or 8 of Table II, application no. 14, or a homolog or a fragment thereof, a consensus sequence or at least one polypeptide motif as depicted in the respective line in column 8 of Table IV, application no. 14, whereby the respective line disclose in column 7 the fine chemical stigmasterol; or
increasing or generating the activity of an expression product of one or more nucleic acid molecule(s) comprising a polynucleotide as depicted in the respective line in column 5 or 8 of Table I, application no. 14, preferably the coding region thereof, or a homolog or a fragment thereof, whereby the respective line disclose in column 7 the fine chemical stigmasterol;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of stigmasterol, or a composition comprising stigmasterol in said non-human organism or in the culture medium surrounding said non-human organism.

A further embodiment of the present invention relates to a process for the production of campesterol, which comprises
(a) increasing or generating one or more activities selected from the group consisting of C-4 sterol methyl oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of eukaryotic translation initiation factor 5 in an organelle, preferably in plastids or mitochondria, especially in plastids, of a non-human organism or a part thereof, preferably a microorganism organism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of ankyrin repeat family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of sterol desaturase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of beta-sitosterol, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of beta-sitosterol, which comprises
(a) increasing or generating one or more activities selected from the group consisting of sec-independent protein translocase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating one or more activities selected from the group consisting of 2-oxoglutarate dehydrogenase E1 subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating one or more activities selected from the group consisting of CTP synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating one or more activities selected from the group consisting of CTP synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of recombinase A in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating one or more activities selected from the group consisting of hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating one or more activities selected from the group consisting of hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of threonine synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the pro-duction of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of L-ribulose-5-phosphate 4-epimerase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of glucose dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating one or more activities selected from the group consisting of ATP-binding component of a transport system in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating one or more activities selected from the group consisting of ATP-binding component of a transport system in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of membrane transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of putrescine transport system permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating one or more activities selected from the group consisting of multidrug resistance protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of beta-sitosterol, which comprises
(a) increasing or generating one or more activities selected from the group consisting of sodium/proton antiporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating one or more activities selected from the group consisting of sodium/proton antiporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating one or more activities selected from the group consisting of phenylacetic acid degradation protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating one or more activities selected from the group consisting of phenylacetic acid degradation protein in an organelle, preferably in plastids or mitochondria, especially in plastids, of a non-human organism or a part thereof, preferably a microorganism organism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of b1401-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of lipoprotein precursor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating one or more activities selected from the group consisting of acid shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating one or more activities selected from the group consisting of acid shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of B1795-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of b2107-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of NADH dehydrogenase I chain N in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of amidophosphoribosyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of b2376-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of b2399-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of b2474-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of b2513-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating one or more activities selected from the group consisting of putative transport system permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating one or more activities selected from the group consisting of putative transport system permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of b2654-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of murein transglycosylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of xanthine dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of b3151-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating one or more activities selected from the group consisting of methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating one or more activities selected from the group consisting of threonine dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating one or more activities selected from the group consisting of b3989-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating one or more activities selected from the group consisting of b3989-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating one or more activities selected from the group consisting of b4029-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of stigmasterol, which comprises
(a) increasing or generating one or more activities selected from the group consisting of b4029-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stigmasterol or a composition comprising stigmasterol in said non-human organism or in the culture medium surrounding said non-human organism.

A further embodiment of the present invention relates to a process for the production of beta-sitosterol, which comprises
(a) increasing or generating one or more activities selected from the group consisting of b4205-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of beta-sitosterol, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the pro-duction of beta-sitosterol or a composition comprising beta-sitosterol in said non-human organism or in the culture medium surrounding said non-human organism.

A further embodiment of the present invention relates to a process for the production of campesterol, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of sll0406-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of cation transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating one or more activities selected from the group consisting of CTP synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating one or more activities selected from the group consisting of CTP synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating one or more activities selected from the group consisting of phosphoribulokinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of beta-sitosterol, which comprises
(a) increasing or generating one or more activities selected from the group consisting of ABC metal ion transporter substrate-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating one or more activities selected from the group consisting of sll1761-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating one or more activities selected from the group consisting of acyl transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating one or more activities selected from the group consisting of cation-transporting ATPase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating one or more activities selected from the group consisting of permease protein of ABC transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of tryptophan synthase alpha chain in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating one or more activities selected from the group consisting of multiple antibiotic resistance protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating one or more activities selected from the group consisting of multiple antibiotic resistance protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating one or more activities selected from the group consisting of TTC1386-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating one or more activities selected from the group consisting of TTC1386-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of homocitrate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of homocitrate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of cyclin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of ydl235c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of polygalacturonase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of campesterol, which comprises
(a) increasing or generating one or more activities selected from the group consisting of ynl321w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a) increasing or generating one or more activities selected from the group consisting of acetyl-CoA acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of beta-sitosterol, which comprises
(a) increasing or generating one or more activities selected from the group consisting of ypr170c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

A further embodiment of the present invention relates to a process for the production of campesterol, which comprises
(a) increasing or generating one or more activities selected from the group consisting of glucose-6-phosphate 1-dehydrogenase in an organelle, preferably in plastids or mitochondria, especially in plastids, of a non-human organism or a part thereof, preferably a microorganism organism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

A further embodiment of the present invention relates to a process for the production of beta-sitosterol, which comprises
(a1) increasing or generating the activity of a polypeptide comprising a polypeptide as depicted in the respective line in column 5 or 8 of Table II, application no. 14, or a homolog or fragment thereof, a consensus sequence or at least one polypeptide motif as depicted in column 8 of Table IV, application no. 14, whereby the respective line disclose in column 7 the fine chemical beta-sitosterol; or
increasing or generating the activity of an expression product of one or more nucleic acid molecule(s) comprising a polynucleotide as depicted in the respective line in column 5 or 8 of Table I, application no. 14, preferably the coding region thereof, or a homolog or a fragment thereof, whereby the respective line disclose in column 7 the fine chemical beta-sitosterol;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 14, or a homolog or a fragment thereof, a consensus sequence or at least one polypeptide motif as depicted in the respective line in column 8 of Table IV, application no. 14, which is joined to a transit peptide, whereby the respective line disclose in column 7 the fine chemical beta-sitosterol; or
increasing or generating the activity of an expression product of one or more nucleic acid molecule(s) comprising a polynucleotide as depicted in the respective line in column 5 or 8 of Table I, application no. 14, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical beta-sitosterol;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 14, or a homolog or a fragment thereof, a consensus sequence or at least one polypeptide motif as depicted in the respective line in column 8 of Table IV, application no. 14, whereby the respective line disclose in column 7 the fine chemical beta-sitosterol; or
increasing or generating the activity of an expression product of one or more nucleic acid molecule(s) comprising a polynucleotide as depicted in the respective line in column 5 or 8 of Table I, application no. 14, preferably the coding region thereof, or a homolog or a fragment thereof, whereby the respective line disclose in column 7 the fine chemical beta-sitosterol;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of beta-sitosterol, or a composition comprising beta-sitosterol in said non-human organism or in the culture medium surrounding said non-human organism.

A further embodiment of the present invention relates to a process for the production of campesterol, which comprises
(a1) increasing or generating the activity of a polypeptide comprising a polypeptide as depicted in the respective line in column 5 or 8 of Table II, application no. 14, or a homolog or fragment thereof, a consensus sequence or at least one polypeptide motif as depicted in column 8 of Table IV, application no. 14, whereby the respective line disclose in column 7 the fine chemical campesterol; or
increasing or generating the activity of an expression product of one or more nucleic acid molecule(s) comprising a polynucleotide as depicted in the respective line in column 5 or 8 of Table I, application no. 14, preferably the coding region thereof, or a homolog or a fragment thereof, whereby the respective line disclose in column 7 the fine chemical campesterol;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 14, or a homolog or a fragment thereof, a consensus sequence or at least one polypeptide motif as depicted in the respective line in column 8 of Table IV, application no. 14, which is joined to a transit peptide, whereby the respective line disclose in column 7 the fine chemical campesterol; or
increasing or generating the activity of an expression product of one or more nucleic acid molecule(s) comprising a polynucleotide as depicted in the respective line in column 5 or 8 of Table I, application no. 14, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical campesterol;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 14, or a homolog or a fragment thereof, a consensus sequence or at least one polypeptide motif as depicted in the respective line in column 8 of Table IV, application no. 14, whereby the respective line disclose in column 7 the fine chemical campesterol; or
increasing or generating the activity of an expression product of one or more nucleic acid molecule(s) comprising a polynucleotide as depicted in the respective line in column 5 or 8 of Table I, application no. 14, preferably the coding region thereof, or a homolog or a fragment thereof, whereby the respective line disclose in column 7 the fine chemical campesterol;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of campesterol, or a composition comprising campesterol in said non-human organism or in the culture medium surrounding said non-human organism.

A further embodiment of the present invention relates to a process for the production of stigmasterol, which comprises
(a1) increasing or generating the activity of a polypeptide comprising a polypeptide as depicted in the respective line in column 5 or 8 of Table II, application no. 14, or a homolog or fragment thereof, a consensus sequence or at least one polypeptide motif as depicted in column 8 of Table IV, application no. 14, whereby the respective line disclose in column 7 the fine chemical stigmasterol; or
increasing or generating the activity of an expression product of one or more nucleic acid molecule(s) comprising a polynucleotide as depicted in the respective line in column 5 or 8 of Table I, application no. 14, preferably the coding region thereof, or a homolog or a fragment thereof, whereby the respective line disclose in column 7 the fine chemical stigmasterol;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 14, or a homolog or a fragment thereof, a consensus sequence or at least one polypeptide motif as depicted in the respective line in column 8 of Table IV, application no. 14, which is joined to a transit peptide, whereby the respective line disclose in column 7 the fine chemical stigmasterol; or
increasing or generating the activity of an expression product of one or more nucleic acid molecule(s) comprising a polynucleotide as depicted in the respective line in column 5 or 8 of Table I, application no. 14, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical stigmasterol;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 14, or a homolog or a fragment thereof, a consensus sequence or at least one polypeptide motif as depicted in the respective line in column 8 of Table IV, application no. 14, whereby the respective line disclose in column 7 the fine chemical stigmasterol; or
increasing or generating the activity of an expression product of one or more nucleic acid molecule(s) comprising a polynucleotide as depicted in the respective line in column 5 or 8 of Table I, application no. 14, preferably the coding region thereof, or a homolog or a fragment thereof, whereby the respective line disclose in column 7 the fine chemical stigmasterol;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of stigmasterol, or a composition comprising stigmasterol in said non-human organism or in the culture medium surrounding said non-human organism.

Accordingly, the present invention relates to a process for the production of campesterol, which comprises
(a) increasing or generating the activity of a C-4 sterol methyl oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a eukaryotic translation initiation factor 5 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a ankyrin repeat family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a sterol desaturase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of beta-sitosterol, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of beta-sitosterol, which comprises
(a) increasing or generating the activity of a sec-independent protein translocase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating the activity of a 2-oxoglutarate dehydrogenase E1 subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating the activity of a CTP synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating the activity of a CTP synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a recombinase A in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating the activity of a hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating the activity of a hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a threonine synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a L-ribulose-5-phosphate 4-epimerase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a glucose dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating the activity of a ATP-binding component of a transport system in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating the activity of a ATP-binding component of a transport system in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a membrane transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a putrescine transport system permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating the activity of a multidrug resistance protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of beta-sitosterol, which comprises
(a) increasing or generating the activity of a sodium/proton antiporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating the activity of a sodium/proton antiporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating the activity of a phenylacetic acid degradation protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating the activity of a phenylacetic acid degradation protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a b1401-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a lipoprotein precursor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating the activity of a acid shock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating the activity of a acid shock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a B1795-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a b2107-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a NADH dehydrogenase I chain N in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a amidophosphoribosyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a b2376-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a b2399-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a b2474-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a b2513-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating the activity of a putative transport system permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating the activity of a putative transport system permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a b2654-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a murein transglycosylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a xanthine dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a b3151-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating the activity of a methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating the activity of a threonine dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating the activity of a b3989-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating the activity of a b3989-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating the activity of a b4029-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of stigmasterol, which comprises
(a) increasing or generating the activity of a b4029-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of stigmasterol or a composition comprising stigmasterol in said non-human organism or in the culture medium surrounding said non-human organism.

Accordingly, the present invention relates to a process for the production of beta-sitosterol, which comprises
(a) increasing or generating the activity of a b4205-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of beta-sitosterol, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a sll0406-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a cation transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating the activity of a CTP synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating the activity of a CTP synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating the activity of a phosphoribulokinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of beta-sitosterol, which comprises
(a) increasing or generating the activity of a ABC metal ion transporter substrate-binding protein in the cytosol of a cell of a non-human organism or a part thereof, preferably a microorganism organism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating the activity of a sll1761-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating the activity of a acyl transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating the activity of a cation-transporting ATPase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating the activity of a permease protein of ABC transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a tryptophan synthase alpha chain in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating the activity of a multiple antibiotic resistance protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating the activity of a multiple antibiotic resistance protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating the activity of a TTC1386-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating the activity of a TTC1386-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a homocitrate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a homocitrate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a cyclin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a ydl235c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a polygalacturonase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of campesterol, which comprises
(a) increasing or generating the activity of a ynl321w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating the activity of a acetyl-CoA acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of beta-sitosterol, which comprises
(a) increasing or generating the activity of a ypr170c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol or a composition comprising beta-sitosterol in said non-human organism or in the culture medium surrounding said non-human organism.

Accordingly, the present invention relates to a process for the production of campesterol, which comprises
(a) increasing or generating the activity of a glucose-6-phosphate 1-dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of campesterol or a composition comprising campesterol in said non-human organism or in the culture medium surrounding said non-human organism.

Accordingly, the present invention relates to a process for the production of beta-sitosterol, which comprises
(a) increasing or generating the activity of a polypeptide comprising a polypeptide as depicted in the respective line in column 5 or 8 of Table II, application no. 14, or a homolog or a fragment thereof, a consensus sequence or at least one polypeptide motif as depicted in the respective line in column 8 of Table IV, application no. 14, whereby the respective line disclose in column 7 the fine chemical beta-sitosterol; or
increasing or generating the activity of an expression product of one or more nucleic acid molecule(s) comprising a polynucleotide as depicted in the respective line in column 5 or 8 of Table I, application no. 14, preferably the coding region thereof, or a homolog or a fragment thereof, whereby the respective line disclose in column 7 the fine chemical beta-sitosterol;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of beta-sitosterol, or a composition comprising beta-sitosterol in said non-human organism or in the culture medium surrounding said non-human organism.

Accordingly, the present invention relates to a process for the production of campesterol, which comprises
(a) increasing or generating the activity of a polypeptide comprising a polypeptide as depicted in the respective line in column 5 or 8 of Table II, application no. 14, or a homolog or a fragment thereof, a consensus sequence or at least one polypeptide motif as depicted in the respective line in column 8 of Table IV, application no. 14, whereby the respective line disclose in column 7 the fine chemical campesterol; or
increasing or generating the activity of an expression product of one or more nucleic acid molecule(s) comprising a polynucleotide as depicted in the respective line in column 5 or 8 of Table I, application no. 14, preferably the coding region thereof, or a homolog or a fragment thereof, whereby the respective line disclose in column 7 the fine chemical campesterol;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of campesterol, or a composition comprising campesterol in said non-human organism or in the culture medium surrounding said non-human organism.

Accordingly, the present invention relates to a process for the production of stigmasterol, which comprises
(a) increasing or generating the activity of a polypeptide comprising a polypeptide as depicted in the respective line in column 5 or 8 of Table II, application no. 14, or a homolog or a fragment thereof, a consensus sequence or at least one polypeptide motif as depicted in the respective line in column 8 of Table IV, application no. 14, whereby the respective line disclose in column 7 the fine chemical stigmasterol; or
increasing or generating the activity of an expression product of one or more nucleic acid molecule(s) comprising a polynucleotide as depicted in the respective line in column 5 or 8 of Table I, application no. 14, preferably the coding region thereof, or a homolog or a fragment thereof, whereby the respective line disclose in column 7 the fine chemical stigmasterol;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of stigmasterol, or a composition comprising stigmasterol in said non-human organism or in the culture medium surrounding said non-human organism.

In a further embodiment the activity of the polypeptide comprising a polypeptide as depicted in the respective line in column 5 or 8 of Table II, application no. 14, or a homolog or a fragment thereof, a consensus sequence or at least one polypeptide motif as depicted in the respective line in column 8 of Table IV, application no. 14, is increased or generated non-targeted in the above-mentioned process in a microorganism or plant or a part thereof.

In a further embodiment said polypeptide has the activity of the respective polypeptide represented by a protein comprising a polypeptide as depicted in the respective line in column 5 of Table II, application no. 14.

In a further embodiment the activity of the expression product of one or more nucleic acid molecule(s) comprising a polynucleotide as depicted in the respective line in column 5 or 8 of Table I, application no. 14, preferably the coding region thereof, or a homolog or a fragment thereof, is increased or generated non-targeted in the above-mentioned process in a microorganism or plant or a part thereof.

In a further embodiment the activity of the polypeptide comprising a polypeptide as depicted in the respective line in column 5 or 8 of Table II, application no. 14, or a homolog or a fragment thereof, a consensus sequence or at least one polypeptide motif as depicted in the respective line in column 8 of Table IV, application no. 14, is increased or generated in the above-mentioned process in an organelle preferably in plastids or mitochondria, especially in plastids, of a microorganism or plant.

In a further embodiment said polypeptide has the activity of the respective polypeptide represented by a protein comprising a polypeptide as depicted in the respective line in column 5 of Table II, application no. 14.

In a further embodiment the activity of the expression product of one or more nucleic acid molecule(s) comprising a polynucleotide as depicted in the respective line in column 5 or 8 of Table I, application no. 14, preferably the coding region thereof, or a homolog or a fragment thereof, is increased or generated in the above-mentioned process in an organelle, preferably in plasids or mitochondria, especially in plastids, of a microorganism or plant.

In a further embodiment the activity of the polypeptide comprising a polypeptide as depicted in the respective line in column 5 or 8 of Table II, application no. 14, or a homolog or a fragment thereof, a consensus sequence or at least one polypeptide motif as depicted in the respective line in column 8 of Table IV, application no. 14, is increased or generated in the above-mentioned process in the cyctosol of a cell, of a microorganism or plant.

In a further embodiment said polypeptide has the activity of the respective polypeptide represented by a protein comprising a polypeptide as depicted in the respective line in column 5 of Table II, application no. 14.

In a further embodiment the activity of the expression product of one or more nucleic acid molecule(s) comprising a polynucleotide as depicted in the respective line in column 5 or 8 of Table I, application no. 14, preferably the coding region thereof, or a homolog or a fragment thereof, is increased or generated in the above-mentioned process in the cytosol of a cell, of a microorganism or plant.

[0039.1.7.14] to [0066.1.7.14] for the disclosure of these paragraphs see [0039.1.7.7] to [0066.1.7.7] above

As mentioned above the nucleic acid sequences coding for the proteins as shown in Table II, application no. 14, column 5 or 8, or homologs or fragments thereof, are joined to a nucleic acid sequence encoding a plastidic transit peptide, e.g, if for the nucleic acid molecule in Table I, column 6 the term “plastidic” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide coding sequence is fused in frame to the nucleic acid sequence coding for proteins as shown in Table II, application no. 14, column 5 or 8, or homologs or fragments thereof, e.g., if for the nucleic acid molecule in Table I, column 6 the term “plastidic” is indicated.

[0068.1.7.14] to [0072.1.7.14] for the disclosure of these paragraphs see [0068.1.7.7] to [0072.1.7.7] above

As mentioned above the nucleic acid sequences coding for the proteins as shown in the respective line in Table II, application no. 14, column 5 or 8, or homologs or fragments thereof, are joined to a nucleic acid sequence encoding a mitochondric transit peptide, e.g., if for the respective nucleic acid molecule in Table I, column 6 the term “mitochondric” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in the respective line in Table II, application no. 14, column 5 or 8, or homologs or fragments thereof, e.g., if for the nucleic acid molecule in the respective line in Table I, column 6 the term “mitochondric”. is indicated.

for the disclosure of this paragraph see [0074.1.7.7] above

for the disclosure of this paragraph see [0075.1.7.7] above

Nucleic acid sequences coding for the transit peptides may be chemically synthesized either in part or wholly according to structure of transit peptide sequences disclosed in the prior art. Said natural or chemically synthesized sequences can be directly linked to the sequences encoding the mature protein or via a linker nucleic acid sequence, which may be typically less than 500 base pairs, preferably less than 450, 400, 350, 300, 250 or 200 base pairs, more preferably less than 150, 100, 90, 80, 70, 60, 50, 40 or 30 base pairs and most preferably less than 25, 20, 15, 12, 9, 6 or 3 base pairs in length and are in frame to the coding sequence. Furthermore favorable nucleic acid sequences encoding transit peptides may comprise sequences derived from more than one biological and/or chemical source and may include a nucleic acid sequence derived from the amino-terminal region of the mature protein, which in its native state is linked to the transit peptide. In a preferred embodiment of the invention said amino-terminal region of the mature protein is typically less than 150 amino acids, preferably less than 140, 130, 120, 110, 100 or 90 amino acids, more preferably less than 80, 70, 60, 50, 40, 35, 30, 25 or 20 amino acids and most preferably less than 19, 18, 17, 16, 15, 14, 13, 12, 11 or 10 amino acids in length. But even shorter or longer stretches are also possible. In addition target sequences, which facilitate the transport of proteins to other cell compartments such as the vacuole, endoplasmic reticulum, golgi complex, glyoxysomes, peroxisomes, mitochondria (in case of a plastidal transit peptide) or plastids, especially chloroplasts (in case of a mitochondric transit peptide) may be also part of the inventive nucleic acid sequence. The proteins translated from said inventive nucleic acid sequences are a kind of fusion proteins that means the nucleic acid sequences encoding the plastidal transit peptide for example the ones shown in Table a, preferably the last one of the Table a are joint to the respective nucleic acid sequences shown in Table I, application no. 14, columns 5 or 8, especially the coding region thereof, or homologs or fragments thereof, in case of nucleic acid sequences being expressed in plastids, and that means the nucleic acid sequences encoding the mitochondric transit peptide for example the ones shown in Table b, preferably the last one of the Table b are joint to the respective nucleic acid sequences shown in Table I, application no. 14, columns 5 or 8, especially the coding region thereof, or homologs or fragments thereof, in case of nucleic acid sequences being expressed in mitochondria. The person skilled in the art is able to join said sequences in a functional manner. Advantageously the transit peptide part is cleaved off from the protein part shown in Table II, application no. 14, columns 5 or 8, or a homolog or fragment thereof, during or after the transport into the organelles, preferably into the plastids or mitochondria, respectively.

All products of the cleavage of the preferred transit peptide shown in the last line of Table a have preferably the N-terminal amino acid sequences QIA CSS or QIA EFQLTT in front of the start methionine of the respective protein mentioned in Table II, application no. 14, column 5 or 8. or homologs or fragments thereof. Other short amino acid sequences of an range of 1 to 20 amino acids preferable 2 to 15 amino acids, more preferable 3 to 10 amino acids most preferably 4 to 8 amino acids are also possible in front of the start methionine of the protein mentioned in Table II, application no. 14, columns 5 or 8, or homologs or fragments thereof. In case of the amino acid sequence QIA CSS the three amino acids in front of the start methionine are stemming from the LIC (=ligatation independent cloning) cassette. Said short amino acid sequence is preferred in the case of the expression of E. coli genes. In case of the amino acid sequence QIA EFQLTT the six amino acids in front of the start methionine are stemming from the LIC cassette. Said short amino acid sequence is preferred in the case of the expression of S. cerevisiae genes. The skilled worker knowns that other short sequences are also useful in the expression of the respective nucleic acid molecule comprising a polynucleotide as depicted in Table I, application no. 14, column 5 or 8, preferably the coding region thereof, or a homolog or a fragment thereof. Furthermore the skilled worker is aware of the fact that there is not a need for such short sequences in the expression of the genes.

for the disclosure of this paragraph see [0077.1.7.7] above

for the disclosure of this paragraph see [0078.1.7.7] above

Alternatively to the targeting of the respective sequences shown in Table II, application no. 14, columns 5 or 8, or homologs or fragments thereof, preferably of sequences in general encoded in the nucleus with the aid of the targeting sequences mentioned for example in Table a alone, Table b alone, or in combination with other targeting sequences preferably into plastids or mitochondria, respectively, the nucleic acids of the invention can directly be introduced into the plastidal or mitochondrial genome. Therefore in a preferred embodiment the respective nucleic acid sequences shown in the respective line in Table I, application no. 14, columns 5 or 8, preferably the coding region thereof, or homologs or fragments thereof, are directly introduced and expressed in plastids e.g., if for the nucleic acid molecule in the respective line in Table I, column 6, the term “plastidic” is indicated, or are directly introduced and expressed in mitochondria e.g., if for the nucleic acid molecule in the respective line in Table I, column 6 the term “mitochondric” is indicated.

The term “introduced” in the context of this specification shall mean the insertion of a nucleic acid sequence into the organism by means of a “transfection”, “transduction” or preferably by “transformation”.

A plastid, such as a chloroplast, or a mitochondrion, has been “transformed” by an exogenous (preferably foreign) nucleic acid sequence if a nucleic acid sequence has been introduced into the plastid or mitochondrion that means that this sequence has crossed the membrane or the membranes of the plastid or of the mitochondrion. The exogenous, preferably foreign, DNA may be integrated (covalently linked) into plastid or mitochondrial DNA making up the genome of the plastid or mitochondrion, or it may remain unintegrated (e.g., by including a chloroplast origin of replication). “Stably” integrated DNA sequences are those, which are inherited through plastid or mitochondrion replication, thereby transferring new plastids or mitochondria, with the features of the integrated DNA sequence to the progeny.

[0080.1.7.14] to [0083.1.7.14] for the disclosure of these paragraphs see [0080.1.7.7] to [0083.1.7.7] above

For the process of the present invention it may be of great advantage that by transforming the plastids the intraspecies specific transgene flow is blocked, because a lot of species such as corn, cotton and rice have a strict maternal inheritance of plastids. By placing the respective genes specified in the respective line in Table I, application no. 14, columns 5 or 8, preferably the coding region, or homologs or fragments thereof, e.g. if for the nucleic acid molecule in the respective line in column 6 of Table I the term “plastidic” is indicated, in the plastids of plants, these genes will not be present in the pollen of said plants.

A further preferred embodiment of the invention relates to the use of so called “chloroplast localization sequences”, in which a first RNA sequence or molecule is capable of transporting or “chaperoning” a second RNA sequence, such as a RNA sequence transcribed from the sequences depicted in the respective line in Table I, application no. 14, columns 5 or 8, preferably the coding region thereof, or a homolog or a fragment thereof, or a sequence encoding a protein, as depicted in the respective line in Table II, application no. 14, columns 5 and 8, or a homolog or a fragment thereof, from an external environment inside a cell or outside a plastid into a chloroplast. In one embodiment the chloroplast localization signal is substantially similar or complementary to a complete or intact viroid sequence. The chloroplast localization signal may be encoded by a DNA sequence, which is transcribed into the chloroplast localization RNA. The term “viroid” refers to a naturally occurring single stranded RNA molecule (Flores, C. R. Acad. Sci. III. 324 (10), 943 (2001)). Viroids usually contain about 200-500 nucleotides and generally exist as circular molecules. Examples of viroids that contain chloroplast localization signals include but are not limited to ASBVd, PLMVd, CChMVd and ELVd. The viroid sequence or a functional part of it can be fused to the sequences depicted in the respective line in Table I, application no. 14, columns 5 or 8, preferably the coding region thereof, or a homolog or a fragment thereof, or a sequence encoding a protein, as depicted in the respective line in Table II, application no. 14, columns 5 or 8, or a homolog or a fragment thereof, in such a manner that the viroid sequence transports a sequence transcribed from a sequence as depicted in the respective line in Table I, application no. 14 columns 5 or 8, preferably the coding region thereof, or a homolog or a fragment thereof, or a sequence encoding a protein as depicted in the respective line in Table II, application no. 14 columns 5 or 8, or a homolog or a fragment thereof, into the chloroplasts, e.g. if for the nucleic acid molecule in column 6 of Table I the term “plastidic” is indicated. A preferred embodiment uses a modified ASBVd (Navarro et al., Virology. 268 (1), 218 (2000)).

In a further specific embodiment the protein to be expressed in the plastids such as the proteins depicted in the respective line in Table II, application no. 14, columns 5 or 8, or a homolog or a fragment thereof, e.g. if for the nucleic acid molecule in the respective line in column 6 of Table I the term “plastidic” is indicated, are encoded by different nucleic acids. Such a method is disclosed in WO 2004/040973, which shall be incorporated by reference. WO 2004/040973 teaches a method, which relates to the translocation of an RNA corresponding to a gene or gene fragment into the chloroplast by means of a chloroplast localization sequence. The genes, which should be expressed in the plant or plant cells, are split into nucleic acid fragments, which are introduced into different compartments in the plant e.g. the nucleus, the plastids and/or mitochondria. Additionally plant cells are described in which the chloroplast contains a ribozyme fused at one end to an RNA encoding a fragment of a protein used in the inventive process such that the ribozyme can trans-splice the translocated fusion RNA to the RNA encoding the gene fragment to form and as the case may be reunite the nucleic acid fragments to an intact mRNA encoding a functional protein for example as disclosed in the respective line in Table II, application no. 14, columns 5 or 8, or a homolog or a fragment thereof.

In a preferred embodiment of the invention the nucleic acid sequences as shown in the respective line in Table I, application no. 14, columns 5 or 8, preferably the coding region thereof, or homologs or fragments thereof, e.g. if for the nucleic acid molecule in the respective line in column 6 of Table I the term “plastidic” is indicated, used in the inventive process are transformed into plastids, which are metabolically active. Those plastids should preferably maintain at a high copy number in the plant or plant tissue of interest, most preferably the chloroplasts found in green plant tissues, such as leaves or cotyledons or in seeds.

For a good expression in the plastids the nucleic acid sequences as shown in the respective line in Table I, application no. 14, columns 5 or 8, preferably the coding region thereof, or homologs or fragments thereof, e.g. if for the nucleic acid molecule in the respective line in column 6 of Table I the term “plastidic” is indicated, are introduced into an expression cassette using preferably a promoter and terminater, which are active in plastids preferably a chloroplast promoter. Examples of such promoters include the psbA promoter from the gene from spinach or pea, the rbcL promoter, and the atpB promoter from corn.

[0089.1.7.14] to [0092.1.7.14] for the disclosure of these paragraphs see [0089.1.7.7] to [0092.1.7.7] above

Advantageously the process for the production of the fine chemical beta-sitosterol, campesterol, and/or stigmasterol leads to an enhanced production of the respective fine chemical. The terms “enhanced” or “increase” mean at least a 10%, 20%, 30%, 40% or 50%, preferably at least 60%, 70%, 80%, 90% or 100%, more preferably 150%, 200%, 300%, 400% or 500% higher production of the respective fine chemical beta-sitosterol, campesterol, and/or stigmasterol in comparison to the wild-type as defined above, e.g. that means in comparison to a non-human organism without the aforementioned modification of the activity of a protein as shown in the respective line in Table II, application no. 14, column 5 or 8, or a fragment or a homolog thereof. The modification of the activity of a protein as shown in the respective line in Table II, application no. 14, column 5 or 8, or a homolog or a fragment thereof, or their combination can be achieved by joining the protein to a respective transit peptide, e.g. if for the respective encoding nucleic acid molecule in column 6 of Table I the term “plastidic” or “mitochondric” is indicated.

Surprisingly it was found, that the transgenic expression of a protein as shown in the respective line in Table II, application no. 14, column 5, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, conferred a production of or an increase in beta-sitosterol, campesterol, and/or stigmasterol,respectively, to the transgenic non-human organism as compared to a corresponding non-transformed wild type.

In one embodiment the transgenic expression of a protein as shown in the respective line in Table II, application no. 14, column 5 or 8, or a homolog or a fragment thereof, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, confers a production of or an increase in beta-sitosterol, campesterol, and/or stigmasterol,respectively, to the transgenic non-human organism as compared to a corresponding non-transformed wild type.

Accordingly, in one embodiment, an increase of campesterol in a non-human organism, as compared to a corresponding non-transformed wild type non-human organism, is conferred in the process of the invention, if the activity of a polypeptide showing the activity of a C-4 sterol methyl oxidase, or if the activity of the polypeptide At1g07420, preferably represented by SEQ ID NO. 131004, or a homolog or fragment thereof, or if the activity of a polypeptide encoded by a nucleic acid molecule comprising the nucleic acid SEQ ID NO. 131003, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 131003 or polypeptide SEQ ID NO. 131004, respectively, or a homolog or a fragment thereof, is increased or generated, or if the activity C-4 sterol methyl oxidase is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 89 to 300-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a protein phosphatase, or if the activity of the polypeptide At1g07430, preferably represented by SEQ ID NO. 17452, or a homolog or fragment thereof, or if the activity of a polypeptide encoded by a nucleic acid molecule comprising the nucleic acid SEQ ID NO. 17451, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 17451 or polypeptide SEQ ID NO. 17452, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 18 to 54-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a eukaryotic translation initiation factor 5, or if the activity of the polypeptide At1g36730, preferably represented by SEQ ID NO. 17969, or a homolog or fragment thereof, or if the activity of a polypeptide encoded by a nucleic acid molecule comprising the nucleic acid SEQ ID NO. 17968, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 17968 or polypeptide SEQ ID NO. 17969, respectively, or a homolog or a fragment thereof, is increased or generated, or if the activity eukaryotic translation initiation factor 5 is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 17 to 36-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a calcium-dependent protein kinase, or if the activity of the polypeptide At1g61950, preferably represented by SEQ ID NO. 18870, or a homolog or fragment thereof, or if the activity of a polypeptide encoded by a nucleic acid molecule comprising the nucleic acid SEQ ID NO. 18869, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 18869 or polypeptide SEQ ID NO. 18870, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line disclose in column 7 the fine chemical beta-sitosterol. For example, an increase of the beta-sitosterol of at least 1 percent, particularly in a range of 17 to 22-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a calcium-dependent protein kinase, or if the activity of the polypeptide At1g61950, preferably represented by SEQ ID NO. 18870, or a homolog or fragment thereof, or if the activity of a polypeptide encoded by a nucleic acid molecule comprising the nucleic acid SEQ ID NO. 18869, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 18869 or polypeptide SEQ ID NO. 18870, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 17 to 37-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a transcription factor, or if the activity of the polypeptide At1g68320, preferably represented by SEQ ID NO. 1062, or a homolog or fragment thereof, or if the activity of a polypeptide encoded by a nucleic acid molecule comprising the nucleic acid SEQ ID NO. 1061, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 1061 or polypeptide SEQ ID NO. 1062, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical beta-sitosterol. For example, an increase of the beta-sitosterol of at least 1 percent, particularly in a range of 14 to 49-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a transcription factor, or if the activity of the polypeptide At1g68320, preferably represented by SEQ ID NO. 1062, or a homolog or fragment thereof, or if the activity of a polypeptide encoded by a nucleic acid molecule comprising the nucleic acid SEQ ID NO. 1061, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 1061 or polypeptide SEQ ID NO. 1062, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 16 to 76-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a protein phosphatase, or if the activity of the polypeptide At1g72770, preferably represented by SEQ ID NO. 19420, or a homolog or fragment thereof, or if the activity of a polypeptide encoded by a nucleic acid molecule comprising the nucleic acid SEQ ID NO. 19419, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 19419 or polypeptide SEQ ID NO. 19420, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 17 to 79-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a ankyrin repeat family protein, or if the activity of the polypeptide At3g04710, preferably represented by SEQ ID NO. 21903, or a homolog or fragment thereof, or if the activity of a polypeptide encoded by a nucleic acid molecule comprising the nucleic acid SEQ ID NO. 21902, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 21902 or polypeptide SEQ ID NO. 21903, respectively, or a homolog or a fragment thereof, is increased or generated, or if the activity ankyrin repeat family protein is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 16 to 31-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a transcription factor, or if the activity of the polypeptide At4g09960, preferably represented by SEQ ID NO. 109718, or a homolog or fragment thereof, or if the activity of a polypeptide encoded by a nucleic acid molecule comprising the nucleic acid SEQ ID NO. 109717, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 109717 or polypeptide SEQ ID NO. 109718, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical beta-sitosterol. For example, an increase of the beta-sitosterol of at least 1 percent, particularly in a range of 17 to 154-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a transcription factor, or if the activity of the polypeptide At4g09960, preferably represented by SEQ ID NO. 109718, or a homolog or fragment thereof, or if the activity of a polypeptide encoded by a nucleic acid molecule comprising the nucleic acid SEQ ID NO. 109717, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 109717 or polypeptide SEQ ID NO. 109718, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 17 to 38-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a sterol desaturase, or if the activity of the polypeptide At4g12110, preferably represented by SEQ ID NO. 131209, or a homolog or fragment thereof, or if the activity of a polypeptide encoded by a nucleic acid molecule comprising the nucleic acid SEQ ID NO. 131208, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 131208 or polypeptide SEQ ID NO. 131209, respectively, or a homolog or a fragment thereof, is increased or generated, or if the activity sterol desaturase is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 22 to 75-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical beta-sitosterol. For example, an increase of the beta-sitosterol of at least 1 percent, particularly in a range of 15 to 44-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a protein phosphatase, or if the activity of the polypeptide At4g26080, preferably represented by SEQ ID NO. 68850, or a homolog or fragment thereof, or if the activity of a polypeptide encoded by a nucleic acid molecule comprising the nucleic acid SEQ ID NO. 68849, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 68849 or polypeptide SEQ ID NO. 68850, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 16 to 39-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line disclose in column 7 the fine chemical beta-sitosterol. For example, an increase of the beta-sitosterol of at least 1 percent, particularly in a range of 18 to 34-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 35 to 70-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a protein phosphatase, or if the activity of the polypeptide At5g57050, preferably represented by SEQ ID NO. 5319, or a homolog or fragment thereof, or if the activity of a polypeptide encoded by a nucleic acid molecule comprising the nucleic acid SEQ ID NO. 5318, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 5318 or polypeptide SEQ ID NO. 5319, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 30 to 45-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a protein phosphatase, or if the activity of the polypeptide At5g59220, preferably represented by SEQ ID NO. 25499, or a homolog or fragment thereof, or if the activity of a polypeptide encoded by a nucleic acid molecule comprising the nucleic acid SEQ ID NO. 25498, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 25498 or polypeptide SEQ ID NO. 25499, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical beta-sitosterol. For example, an increase of the beta-sitosterol of at least 1 percent, particularly in a range of 13 to 39-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a protein kinase, or if the activity of the polypeptide At5g66710, preferably represented by SEQ ID NO. 122804, or a homolog or fragment thereof, or if the activity of a polypeptide encoded by a nucleic acid molecule comprising the nucleic acid SEQ ID NO. 122803, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 122803 or polypeptide SEQ ID NO. 122804, respectively, or a homolog or a fragment thereof, is increased or generated, or if the activity protein kinase is increased or generated in a non-human organism, like a microorganism or a plant cell, plant or part thereof, especially with non-targeted localization, whereby the respective line disclose in column 7 the fine chemical beta-sitosterol. For example, an increase of the beta-sitosterol of at least 1 percent, particularly in a range of 14 to 23-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a sec-independent protein translocase, or if the activity of the polypeptide AvinDRAFT_{—}1624, preferably represented by SEQ ID NO. 26197, or a homolog or fragment thereof, or if the activity of a polypeptide encoded by a nucleic acid molecule comprising the nucleic acid SEQ ID NO. 26196, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 26196 or polypeptide SEQ ID NO. 26197, respectively, or a homolog or a fragment thereof, is increased or generated, or if the activity sec-independent protein translocase is increased or generated in a non-human organism, like a microorganism or a plant cell, plant or part thereof, especially with non-targeted localization, whereby the respective line disclose in column 7 the fine chemical beta-sitosterol. For example, an increase of the beta-sitosterol of at least 1 percent, particularly in a range of 13 to 18-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a 2-oxoglutarate dehydrogenase E1 subunit, or if the activity of the polypeptide AvinDRAFT_{—}3028, preferably represented by SEQ ID NO. 6076, or a homolog or fragment thereof, or if the activity of a polypeptide encoded by a nucleic acid molecule comprising the nucleic acid SEQ ID NO. 6075, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 6075 or polypeptide SEQ ID NO. 6076, respectively, or a homolog or a fragment thereof, is increased or generated, or if the activity 2-oxoglutarate dehydrogenase E1 subunit is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 18 to 43-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a oxidoreductase, or if the activity of the polypeptide AvinDRAFT_{—}3253, preferably represented by SEQ ID NO. 29501, or a homolog or fragment thereof, or if the activity of a polypeptide encoded by a nucleic acid molecule comprising the nucleic acid SEQ ID NO. 29500, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 29500 or polypeptide SEQ ID NO. 29501, respectively, or a homolog or a fragment thereof, is increased or generated, or if the activity oxidoreductase is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 16 to 35 percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a CTP synthase, or if the activity of the polypeptide AvinDRAFT_{—}3605, preferably represented by SEQ ID NO. 31027, or a homolog or fragment thereof, or if the activity of a polypeptide encoded by a nucleic acid molecule comprising the nucleic acid SEQ ID NO. 31026, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 31026 or polypeptide SEQ ID NO. 31027, respectively, or a homolog or a fragment thereof, is increased or generated, or if the activity CTP synthase is increased or generated in a non-human organism, like a microorganism or a plant cell, plant or part thereof, especially with non-targeted localization, whereby the respective line disclose in column 7 the fine chemical beta-sitosterol. For example, an increase of the beta-sitosterol of at least 1 percent, particularly in a range of 13 to 24-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a CTP synthase, or if the activity of the polypeptide AvinDRAFT_{—}3605, preferably represented by SEQ ID NO. 31027, or a homolog or fragment thereof, or if the activity of a polypeptide encoded by a nucleic acid molecule comprising the nucleic acid SEQ ID NO. 31026, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 31026 or polypeptide SEQ ID NO. 31027, respectively, or a homolog or a fragment thereof, is increased or generated, or if the activity CTP synthase is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 17 to 44-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a recombinase A, or if the activity of the polypeptide AvinDRAFT_{—}3629, preferably represented by SEQ ID NO. 114232, or a homolog or fragment thereof, or if the activity of a polypeptide encoded by a nucleic acid molecule comprising the nucleic acid SEQ ID NO. 114231, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 114231 or polypeptide SEQ ID NO. 114232, respectively, or a homolog or a fragment thereof, is increased or generated, or if the activity recombinase A is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 16 to 72-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a hydrolase, or if the activity of the polypeptide AvinDRAFT_{—}5103, preferably represented by SEQ ID NO. 6511, or a homolog or fragment thereof, or if the activity of a polypeptide encoded by a nucleic acid molecule comprising the nucleic acid SEQ ID NO. 6510, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 6510 or polypeptide SEQ ID NO. 6511, respectively, or a homolog or a fragment thereof, is increased or generated, or if the activity hydrolase is increased or generated in a non-human organism, like a microorganism or a plant cell, plant or part thereof, especially with non-targeted localization, whereby the respective line disclose in column 7 the fine chemical beta-sitosterol. For example, an increase of the beta-sitosterol of at least 1 percent, particularly in a range of 16 to 32-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a hydrolase, or if the activity of the polypeptide AvinDRAFT_{—}5103, preferably represented by SEQ ID NO. 6511, or a homolog or fragment thereof, or if the activity of a polypeptide encoded by a nucleic acid molecule comprising the nucleic acid SEQ ID NO. 6510, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 6510 or polypeptide SEQ ID NO. 6511, respectively, or a homolog or a fragment thereof, is increased or generated, or if the activity hydrolase is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 19 to 45-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a threonine synthase, or if the activity of the polypeptide B0004, preferably represented by SEQ ID NO. 34890, or a homolog or fragment thereof, or if the activity of a polypeptide encoded by a nucleic acid molecule comprising the nucleic acid SEQ ID NO. 34889, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 34889 or polypeptide SEQ ID NO. 34890, respectively, or a homolog or a fragment thereof, is increased or generated, or if the activity threonine synthase is increased or generated in a non-human organism, like a microorganism or a plant cell, plant or part thereof, especially with plastidic localization, whereby the respective line disclose in column 7 the fine chemical campesterol. For example, an increase of the campesterol of at least 1 percent, particularly in a range of 21 to 90-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a L-ribulose-5-phosphate 4-epimerase, or if the activity of the polypeptide B0061, preferably represented by SEQ ID NO. 35205, or a homolog or fragment thereof, or if the activity of a polypeptide encoded by a nucleic acid molecule comprising the nucleic acid SEQ ID NO. 35204, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 35204 or polypeptide SEQ ID NO. 35205, respectively, or a homolog or a fragment thereof, is increased or generated, or if the activity L-ribulose-5-phosphate 4-epimerase is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 16 to 56-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a glucose dehydrogenase, or if the activity of the polypeptide B0124, preferably represented by SEQ ID NO. 35483, or a homolog or fragment thereof, or if the activity of a polypeptide encoded by a nucleic acid molecule comprising the nucleic acid SEQ ID NO. 35482, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 35482 or polypeptide SEQ ID NO. 35483, respectively, or a homolog or a fragment thereof, is increased or generated, or if the activity glucose dehydrogenase is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 26 to 55-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a ATP-binding component of a transport system, or if the activity of the polypeptide B0449, preferably represented by SEQ ID NO. 7334, or a homolog or fragment thereof, or if the activity of a polypeptide encoded by a nucleic acid molecule comprising the nucleic acid SEQ ID NO. 7333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 7333 or polypeptide SEQ ID NO. 7334, respectively, or a homolog or a fragment thereof, is increased or generated, or if the activity ATP-binding component of a transport system is increased or generated in a non-human organism, like a microorganism or a plant cell, plant or part thereof, especially with non-targeted localization, whereby the respective line disclose in column 7 the fine chemical beta-sitosterol. For example, an increase of the beta-sitosterol of at least 1 percent, particularly in a range of 19 to 63-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a ATP-binding component of a transport system, or if the activity of the polypeptide B0449, preferably represented by SEQ ID NO. 7334, or a homolog or fragment thereof, or if the activity of a polypeptide encoded by a nucleic acid molecule comprising the nucleic acid SEQ ID NO. 7333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 7333 or polypeptide SEQ ID NO. 7334, respectively, or a homolog or a fragment thereof, is increased or generated, or if the activity ATP-binding component of a transport system is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 20 to 86-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a membrane transport protein, or if the activity of the polypeptide B0486, preferably represented by SEQ ID NO. 7687, or a homolog or fragment thereof, or if the activity of a polypeptide encoded by a nucleic acid molecule comprising the nucleic acid SEQ ID NO. 7686, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 7686 or polypeptide SEQ ID NO. 7687, respectively, or a homolog or a fragment thereof, is increased or generated, or if the activity membrane transport protein is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 19 to 29-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a putrescine transport system permease protein, or if the activity of the polypeptide B0856, preferably represented by SEQ ID NO. 131360, or a homolog or fragment thereof, or if the activity of a polypeptide encoded by a nucleic acid molecule comprising the nucleic acid SEQ ID NO. 131359, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 131359 or polypeptide SEQ ID NO. 131360, respectively, or a homolog or a fragment thereof, is increased or generated, or if the activity putrescine transport system permease protein is increased or generated in a non-human organism, like a microorganism or a plant cell, plant or part thereof, especially with plastidic localization, whereby the respective line disclose in column 7 the fine chemical campesterol. For example, an increase of the campesterol of at least 1 percent, particularly in a range of 25 to 28-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a multidrug resistance protein, or if the activity of the polypeptide B1065, preferably represented by SEQ ID NO. 76033, or a homolog or fragment thereof, or if the activity of a polypeptide encoded by a nucleic acid molecule comprising the nucleic acid SEQ ID NO. 76032, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 76032 or polypeptide SEQ ID NO. 76033, respectively, or a homolog or a fragment thereof, is increased or generated, or if the activity multidrug resistance protein is increased or generated in a non-human organism, like a microorganism or a plant cell, plant or part thereof, especially with plastidic localization, whereby the respective line disclose in column 7 the fine chemical beta-sitosterol. For example, an increase of the beta-sitosterol of at least 1 percent, particularly in a range of 20 to 27-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a sodium/proton antiporter, or if the activity of the polypeptide B1186, preferably represented by SEQ ID NO. 37401, or a homolog or fragment thereof, or if the activity of a polypeptide encoded by a nucleic acid molecule comprising the nucleic acid SEQ ID NO. 37400, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 37400 or polypeptide SEQ ID NO. 37401, respectively, or a homolog or a fragment thereof, is increased or generated, or if the activity sodium/proton antiporter is increased or generated in a non-human organism, like a microorganism or a plant cell, plant or part thereof, especially with non-targeted localization, whereby the respective line disclose in column 7 the fine chemical beta-sitosterol. For example, an increase of the beta-sitosterol of at least 1 percent, particularly in a range of 15 to 54-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a sodium/proton antiporter, or if the activity of the polypeptide B1186, preferably represented by SEQ ID NO. 37401, or a homolog or fragment thereof, or if the activity of a polypeptide encoded by a nucleic acid molecule comprising the nucleic acid SEQ ID NO. 37400, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 37400 or polypeptide SEQ ID NO. 37401, respectively, or a homolog or a fragment thereof, is increased or generated, or if the activity sodium/proton antiporter is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 16 to 39 percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a phenylacetic acid degradation protein, or if the activity of the polypeptide B1391, preferably represented by SEQ ID NO. 131490, or a homolog or fragment thereof, or if the activity of a polypeptide encoded by a nucleic acid molecule comprising the nucleic acid SEQ ID NO. 131489, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 131489 or polypeptide SEQ ID NO. 131490, respectively, or a homolog or a fragment thereof, is increased or generated, or if the activity phenylacetic acid degradation protein is increased or generated in a non-human organism, like a microorganism or a plant cell, plant or part thereof, especially with non-targeted localization, whereby the respective line disclose in column 7 the fine chemical beta-sitosterol. For example, an increase of the beta-sitosterol of at least 1 percent, particularly in a range of 13 to 24-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a phenylacetic acid degradation protein, or if the activity of the polypeptide B1391, preferably represented by SEQ ID NO. 131490, or a homolog or fragment thereof, or if the activity of a polypeptide encoded by a nucleic acid molecule comprising the nucleic acid SEQ ID NO. 131489, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 131489 or polypeptide SEQ ID NO. 131490, respectively, or a homolog or a fragment thereof, is increased or generated, or if the activity phenylacetic acid degradation protein is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 15 to 29-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a b1401-protein, or if the activity of the polypeptide B1401, preferably represented by SEQ ID NO. 131532, or a homolog or fragment thereof, or if the activity of a polypeptide encoded by a nucleic acid molecule comprising the nucleic acid SEQ ID NO. 131531, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 131531 or polypeptide SEQ ID NO. 131532, respectively, or a homolog or a fragment thereof, is increased or generated, or if the activity b1401-protein is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 19 to 40-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a lipoprotein precursor, or if the activity of the polypeptide B1431, preferably represented by SEQ ID NO. 38267, or a homolog or fragment thereof, or if the activity of a polypeptide encoded by a nucleic acid molecule comprising the nucleic acid SEQ ID NO. 38266, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 38266 or polypeptide SEQ ID NO. 38267, respectively, or a homolog or a fragment thereof, is increased or generated, or if the activity lipoprotein precursor is increased or generated in a non-human organism, like a microorganism organism or a plant cell, plant or part thereof, especially with plastidic localization, whereby the respective line disclose in column 7 the fine chemical campesterol. For example, an increase of the campesterol of at least 1 percent, particularly in a range of 27 to 440-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a acid shock protein, or if the activity of the polypeptide B1597, preferably represented by SEQ ID NO. 38301, or a homolog or fragment thereof, or if the activity of a polypeptide encoded by a nucleic acid molecule comprising the nucleic acid SEQ ID NO. 38300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 38300 or polypeptide SEQ ID NO. 38301, respectively, or a homolog or a fragment thereof, is increased or generated, or if the activity acid shock protein is increased or generated in a non-human organism, like a microorganism or a plant cell, plant or part thereof, especially with non-targeted localization, whereby the respective line disclose in column 7 the fine chemical beta-sitosterol. For example, an increase of the beta-sitosterol of at least 1 percent, particularly in a range of 20 to 33-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a acid shock protein, or if the activity of the polypeptide B1597, preferably represented by SEQ ID NO. 38301, or a homolog or fragment thereof, or if the activity of a polypeptide encoded by a nucleic acid molecule comprising the nucleic acid SEQ ID NO. 38300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 38300 or polypeptide SEQ ID NO. 38301, respectively, or a homolog or a fragment thereof, is increased or generated, or if the activity acid shock protein is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 16 to 46-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a B1795-protein, or if the activity of the polypeptide B1795, preferably represented by SEQ ID NO. 131540, or a homolog or fragment thereof, or if the activity of a polypeptide encoded by a nucleic acid molecule comprising the nucleic acid SEQ ID NO. 131539, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 131539 or polypeptide SEQ ID NO. 131540, respectively, or a homolog or a fragment thereof, is increased or generated, or if the activity B1795-protein is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 37 to 54-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a acetyltransferase, or if the activity of the polypeptide B1932, preferably represented by SEQ ID NO. 131800, or a homolog or fragment thereof, or if the activity of a polypeptide encoded by a nucleic acid molecule comprising the nucleic acid SEQ ID NO. 131799, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 131799 or polypeptide SEQ ID NO. 131800, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 17 to 50-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a b2107-protein, or if the activity of the polypeptide B2107, preferably represented by SEQ ID NO. 39003, or a homolog or fragment thereof, or if the activity of a polypeptide encoded by a nucleic acid molecule comprising the nucleic acid SEQ ID NO. 39002, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 39002 or polypeptide SEQ ID NO. 39003, respectively, or a homolog or a fragment thereof, is increased or generated, or if the activity b2107-protein is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 16 to 48-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a NADH dehydrogenase I chain N, or if the activity of the polypeptide B2276, preferably represented by SEQ ID NO. 131840, or a homolog or fragment thereof, or if the activity of a polypeptide encoded by a nucleic acid molecule comprising the nucleic acid SEQ ID NO. 131839, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 131839 or polypeptide SEQ ID NO. 131840, respectively, or a homolog or a fragment thereof, is increased or generated, or if the activity NADH dehydrogenase I chain N is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 13 to 20-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a amidophosphoribosyltransferase, or if the activity of the polypeptide B2312, preferably represented by SEQ ID NO. 132087, or a homolog or fragment thereof, or if the activity of a polypeptide encoded by a nucleic acid molecule comprising the nucleic acid SEQ ID NO. 132086, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 132086 or polypeptide SEQ ID NO. 132087, respectively, or a homolog or a fragment thereof, is increased or generated, or if the activity amidophosphoribosyltransferase is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 16 to 73-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a b2376-protein, or if the activity of the polypeptide B2376, preferably represented by SEQ ID NO. 132696, or a homolog or fragment thereof, or if the activity of a polypeptide encoded by a nucleic acid molecule comprising the nucleic acid SEQ ID NO. 132695, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 132695 or polypeptide SEQ ID NO. 132696, respectively, or a homolog or a fragment thereof, is increased or generated, or if the activity b2376-protein is increased or generated in a non-human organism, like a microorganism or a plant cell, plant or part thereof, especially with plastidic localization, whereby the respective line disclose in column 7 the fine chemical campesterol. For example, an increase of the campesterol of at least 1 percent, particularly in a range of 26 to 193-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a b2399-protein, or if the activity of the polypeptide B2399, preferably represented by SEQ ID NO. 39238, or a homolog or fragment thereof, or if the activity of a polypeptide encoded by a nucleic acid molecule comprising the nucleic acid SEQ ID NO. 39237, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 39237 or polypeptide SEQ ID NO. 39238, respectively, or a homolog or a fragment thereof, is increased or generated, or if the activity b2399-protein is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 45 to 79-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a b2474-protein, or if the activity of the polypeptide B2474, preferably represented by SEQ ID NO. 40330, or a homolog or fragment thereof, or if the activity of a polypeptide encoded by a nucleic acid molecule comprising the nucleic acid SEQ ID NO. 40329, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 40329 or polypeptide SEQ ID NO. 40330, respectively, or a homolog or a fragment thereof, is increased or generated, or if the activity b2474-protein is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 17 to 48-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a permease protein, or if the activity of the polypeptide B2493, preferably represented by SEQ ID NO. 132707, or a homolog or fragment thereof, or if the activity of a polypeptide encoded by a nucleic acid molecule comprising the nucleic acid SEQ ID NO. 132706, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 132706 or polypeptide SEQ ID NO. 132707, respectively, or a homolog or a fragment thereof, is increased or generated, or if the activity permease protein is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 16 to 29-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a b2513-protein, or if the activity of the polypeptide B2513, preferably represented by SEQ ID NO. 9168, or a homolog or fragment thereof, or if the activity of a polypeptide encoded by a nucleic acid molecule comprising the nucleic acid SEQ ID NO. 9167, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 9167 or polypeptide SEQ ID NO. 9168, respectively, or a homolog or a fragment thereof, is increased or generated, or if the activity b2513-protein is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 37 to 72-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a putative transport system permease protein, or if the activity of the polypeptide B2546, preferably represented by SEQ ID NO. 99899, or a homolog or fragment thereof, or if the activity of a polypeptide encoded by a nucleic acid molecule comprising the nucleic acid SEQ ID NO. 99898, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No.

14, column 5 or 8 in the respective same line as the nucleic acid molecule SEQ ID NO. 99898 or polypeptide SEQ ID NO. 99899, respectively, or a homolog or a fragment thereof, is increased or generated, or if the activity putative transport system permease protein is increased or generated in a non-human organism, like a microorganism or a plant cell, plant or part thereof, especially with non-targeted localization, whereby the respective line disclose in column 7 the fine chemical beta-sitosterol. For example, an increase of the beta-sitosterol of at least 1 percent, particularly in a range of 13 to 21-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a putative transport system permease protein, or if the activity of the polypeptide B2546, preferably represented by SEQ ID NO. 99899, or a homolog or fragment thereof, or if the activity of a polypeptide encoded by a nucleic acid molecule comprising the nucleic acid SEQ ID NO. 99898, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 99898 or polypeptide SEQ ID NO. 99899, respectively, or a homolog or a fragment thereof, is increased or generated, or if the activity putative transport system permease protein is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 16 to 30-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a b2654-protein, or if the activity of the polypeptide B2654, preferably represented by SEQ ID NO.

125227, or a homolog or fragment thereof, or if the activity of a polypeptide encoded by a nucleic acid molecule comprising the nucleic acid SEQ ID NO. 125226, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 125226 or polypeptide SEQ ID NO. 125227, respectively, or a homolog or a fragment thereof, is increased or generated, or if the activity b2654-protein is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 14 to 21-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a murein transglycosylase, or if the activity of the polypeptide B2701, preferably represented by SEQ ID NO. 80757, or a homolog or fragment thereof, or if the activity of a polypeptide encoded by a nucleic acid molecule comprising the nucleic acid SEQ ID NO. 80756, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 80756 or polypeptide SEQ ID NO. 80757, respectively, or a homolog or a fragment thereof, is increased or generated, or if the activity murein transglycosylase is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 16 to 37-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a oxidoreductase, or if the activity of the polypeptide B2766, preferably represented by SEQ ID NO. 132822, or a homolog or fragment thereof, or if the activity of a polypeptide encoded by a nucleic acid molecule comprising the nucleic acid SEQ ID NO. 132821, preferably the coding region thereof, or a homolog or fragment thereof, e.g. derived from Escherichia coli, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 132821 or polypeptide SEQ ID NO. 132822, respectively, or a homolog or a fragment thereof, is increased or generated, or if the activity oxidoreductase is increased or generated in a non-human organism, like a microorganism or a plant cell, plant or part thereof, especially with plastidic localization, whereby the respective line disclose in column 7 the fine chemical campesterol. For example, an increase of the campesterol of at least 1 percent, particularly in a range of 20 to 39-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a xanthine dehydrogenase, or if the activity of the polypeptide B2866, preferably represented by SEQ ID NO. 115865, or a homolog or fragment thereof, or if the activity of a polypeptide encoded by a nucleic acid molecule comprising the nucleic acid SEQ ID NO. 115864, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 115864 or polypeptide SEQ ID NO. 115865, respectively, or a homolog or a fragment thereof, is increased or generated, or if the activity xanthine dehydrogenase is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 30 to 32-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a b3151-protein, or if the activity of the polypeptide B3151, preferably represented by SEQ ID NO. 42478, or a homolog or fragment thereof, or if the activity of a polypeptide encoded by a nucleic acid molecule comprising the nucleic acid SEQ ID NO. 42477, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 42477 or polypeptide SEQ ID NO. 42478, respectively, or a homolog or a fragment thereof, is increased or generated, or if the activity b3151-protein is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 19 to 46-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a methyltransferase, or if the activity of the polypeptide B3262, preferably represented by SEQ ID NO. 42503, or a homolog or fragment thereof, or if the activity of a polypeptide encoded by a nucleic acid molecule comprising the nucleic acid SEQ ID NO. 42502, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 42502 or polypeptide SEQ ID NO. 42503, respectively, or a homolog or a fragment thereof, is increased or generated, or if the activity methyltransferase is increased or generated in a non-human organism, like a microorganism or a plant cell, plant or part thereof, especially with non-targeted localization, whereby the respective line disclose in column 7 the fine chemical beta-sitosterol. For example, an increase of the beta-sitosterol of at least 1 percent, particularly in a range of 12 to 26-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a threonine dehydrogenase, or if the activity of the polypeptide B3616, preferably represented by SEQ ID NO. 42601, or a homolog or fragment thereof, or if the activity of a polypeptide encoded by a nucleic acid molecule comprising the nucleic acid SEQ ID NO. 42600, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 42600 or polypeptide SEQ ID NO. 42601, respectively, or a homolog or a fragment thereof, is increased or generated, or if the activity threonine dehydrogenase is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 19 to 24-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a b3989-protein, or if the activity of the polypeptide B3989, preferably represented by SEQ ID NO. 44373, or a homolog or fragment thereof, or if the activity of a polypeptide encoded by a nucleic acid molecule comprising the nucleic acid SEQ ID NO. 44372, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 44372 or polypeptide SEQ ID NO. 44373, respectively, or a homolog or a fragment thereof, is increased or generated, or if the activity b3989-protein is increased or generated in a non-human organism, like a microorganism or a plant cell, plant or part thereof, especially with non-targeted localization, whereby the respective line disclose in column 7 the fine chemical beta-sitosterol. For example, an increase of the beta-sitosterol of at least 1 percent, particularly in a range of 12 to 27-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a b3989-protein, or if the activity of the polypeptide B3989, preferably represented by SEQ ID NO. 44373, or a homolog or fragment thereof, or if the activity of a polypeptide encoded by a nucleic acid molecule comprising the nucleic acid SEQ ID NO. 44372, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 44372 or polypeptide SEQ ID NO. 44373, respectively, or a homolog or a fragment thereof, is increased or generated, or if the activity b3989-protein is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 37 to 49-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a b4029-protein, or if the activity of the polypeptide B4029, preferably represented by SEQ ID NO. 10741, or a homolog or fragment thereof, or if the activity of a polypeptide encoded by a nucleic acid molecule comprising the nucleic acid SEQ ID NO. 10740, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 10740 or polypeptide SEQ ID NO. 10741, respectively, or a homolog or a fragment thereof, is increased or generated, or if the activity b4029-protein is increased or generated in a non-human organism, like a microorganism or a plant cell, plant or part thereof, especially with non-targeted localization, whereby the respective line disclose in column 7 the fine chemical beta-sitosterol. For example, an increase of the beta-sitosterol of at least 1 percent, particularly in a range of 19 to 78-percent is conferred as compared to a corresponding non-transformed wild type non-human organism.

Accordingly, in one embodiment, an increase of stigmasterol in a non-human organism, as compared to a corresponding non-transformed wild type non-human organism, is conferred in the process of the invention, if the activity of a polypeptide showing the activity of a b4029-protein, or if the activity of the polypeptide B4029, preferably represented by SEQ ID NO. 10741, or a homolog or fragment thereof, or if the activity of a polypeptide encoded by a nucleic acid molecule comprising the nucleic acid SEQ ID NO. 10740, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 10740 or polypeptide SEQ ID NO. 10741, respectively, or a homolog or a fragment thereof, is increased or generated, or if the activity b4029-protein is increased or generated in a non-human organism, like a microorganism or a plant cell, plant or part thereof, especially with non-targeted localization, whereby the respective line disclose in column 7 the fine chemical stigmasterol. For example, an increase of the stigmasterol of at least 1 percent, particularly in a range of 80 to 3076-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a b4205-protein, or if the activity of the polypeptide B4205, preferably represented by SEQ ID NO. 126976, or a homolog or fragment thereof, or if the activity of a polypeptide encoded by a nucleic acid molecule comprising the nucleic acid SEQ ID NO. 126975, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 126975 or polypeptide SEQ ID NO. 126976, respectively, or a homolog or a fragment thereof, is increased or generated, or if the activity b4205-protein is increased or generated in a non-human organism, like a microorganism or a plant cell, plant or part thereof, especially with plastidic localization, whereby the respective line disclose in column 7 the fine chemical beta-sitosterol. For example, an increase of the beta-sitosterol of at least 1 percent, particularly in a range of 13 to 16-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a glutaredoxin, or if the activity of the polypeptide GM02LC12622, preferably represented by SEQ ID NO. 10812, or a homolog or fragment thereof, or if the activity of a polypeptide encoded by a nucleic acid molecule comprising the nucleic acid SEQ ID NO. 10811, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 10811 or polypeptide SEQ ID NO. 10812, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical beta-sitosterol. For example, an increase of the beta-sitosterol of at least 1 percent, particularly in a range of 12 to 37-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a glutaredoxin, or if the activity of the polypeptide GM02LC12622, preferably represented by SEQ ID NO. 10812, or a homolog or fragment thereof, or if the activity of a polypeptide encoded by a nucleic acid molecule comprising the nucleic acid SEQ ID NO. 10811, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 10811 or polypeptide SEQ ID NO. 10812, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 16 to 36-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a transcription factor, or if the activity of the polypeptide GM02LC19289, preferably represented by SEQ ID NO. 46752, or a homolog or fragment thereof, or if the activity of a polypeptide encoded by a nucleic acid molecule comprising the nucleic acid SEQ ID NO. 46751, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 46751 or polypeptide SEQ ID NO. 46752, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical beta-sitosterol. For example, an increase of the beta-sitosterol of at least 1 percent, particularly in a range of 14 to 40-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a transcription factor, or if the activity of the polypeptide GM02LC19289, preferably represented by SEQ ID NO. 46752, or a homolog or fragment thereof, or if the activity of a polypeptide encoded by a nucleic acid molecule comprising the nucleic acid SEQ ID NO. 46751, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 46751 or polypeptide SEQ ID NO. 46752, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 17 to 52-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a cyclin D, or if the activity of the polypeptide GM02LC44512, preferably represented by SEQ ID NO. 46851, or a homolog or fragment thereof, or if the activity of a polypeptide encoded by a nucleic acid molecule comprising the nucleic acid SEQ ID NO. 46850, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 46850 or polypeptide SEQ ID NO. 46851, respectively, or a homolog or a fragment thereof, is increased or generated, or if the activity cyclin D is increased or generated in a non-human organism, like a microorganism or a plant cell, plant or part thereof, especially with non-targeted localization, whereby the respective line disclose in column 7 the fine chemical beta-sitosterol. For example, an increase of the beta-sitosterol of at least 1 percent, particularly in a range of 13 to 30-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a cyclin D, or if the activity of the polypeptide GM02LC44512, preferably represented by SEQ ID NO. 46851, or a homolog or fragment thereof, or if the activity of a polypeptide encoded by a nucleic acid molecule comprising the nucleic acid SEQ ID NO. 46850, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 46850 or polypeptide SEQ ID NO. 46851, respectively, or a homolog or a fragment thereof, is increased or generated, or if the activity cyclin D is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 20 to 56-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a sll0406-protein, or if the activity of the polypeptide Sll0406, preferably represented by SEQ ID NO. 127104, or a homolog or fragment thereof, or if the activity of a polypeptide encoded by a nucleic acid molecule comprising the nucleic acid SEQ ID NO. 127103, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 127103 or polypeptide SEQ ID NO. 127104, respectively, or a homolog or a fragment thereof, is increased or generated, or if the activity sll0406-protein is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 16 to 32-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a cation transport protein, or if the activity of the polypeptide Sll0671, preferably represented by SEQ ID NO. 132875, or a homolog or fragment thereof, or if the activity of a polypeptide encoded by a nucleic acid molecule comprising the nucleic acid SEQ ID NO. 132874, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 132874 or polypeptide SEQ ID NO. 132875, respectively, or a homolog or a fragment thereof, is increased or generated, or if the activity cation transport protein is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 16 to 43-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a CTP synthetase, or if the activity of the polypeptide Sll1443, preferably represented by SEQ ID NO. 53879, or a homolog or fragment thereof, or if the activity of a polypeptide encoded by a nucleic acid molecule comprising the nucleic acid SEQ ID NO. 53878, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 53878 or polypeptide SEQ ID NO. 53879, respectively, or a homolog or a fragment thereof, is increased or generated, or if the activity CTP synthetase is increased or generated in a non-human organism, like a microorganism or a plant cell, plant or part thereof, especially with mitochondrial localization, whereby the respective line disclose in column 7 the fine chemical beta-sitosterol. For example, an increase of the beta-sitosterol of at least 1 percent, particularly in a range of 12 to 25-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a CTP synthetase, or if the activity of the polypeptide Sll1443, preferably represented by SEQ ID NO. 53879, or a homolog or fragment thereof, or if the activity of a polypeptide encoded by a nucleic acid molecule comprising the nucleic acid SEQ ID NO. 53878, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 53878 or polypeptide SEQ ID NO. 53879, respectively, or a homolog or a fragment thereof, is increased or generated, or if the activity CTP synthetase is increased or generated in a non-human organism, like a microorganism or a plant cell, plant or part thereof, especially with mitochondrial localization, whereby the respective line disclose in column 7 the fine chemical campesterol. For example, an increase of the campesterol of at least 1 percent, particularly in a range of 17 to 21-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a phosphoribulokinase, or if the activity of the polypeptide Sll1525, preferably represented by SEQ ID NO. 132939, or a homolog or fragment thereof, or if the activity of a polypeptide encoded by a nucleic acid molecule comprising the nucleic acid SEQ ID NO. 132938, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 132938 or polypeptide SEQ ID NO. 132939, respectively, or a homolog or a fragment thereof, is increased or generated, or if the activity phosphoribulokinase is increased or generated in a non-human organism, like a microorganism or a plant cell, plant or part thereof, especially with mitochondrial localization, whereby the respective line disclose in column 7 the fine chemical beta-sitosterol. For example, an increase of the beta-sitosterol of at least 1 percent, particularly in a range of 17 to 24-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a ABC metal ion transporter substrate-binding protein, or if the activity of the polypeptide Sll1598, preferably represented by SEQ ID NO. 85744, or a homolog or fragment thereof, or if the activity of a polypeptide encoded by a nucleic acid molecule comprising the nucleic acid SEQ ID NO. 85743, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 85743 or polypeptide SEQ ID NO. 85744, respectively, or a homolog or a fragment thereof, is increased or generated, or if the activity ABC metal ion transporter substrate-binding protein is increased or generated in a non-human organism, like a microorganism or a plant cell, plant or part thereof, especially with mitochondrial localization, whereby the respective line disclose in column 7 the fine chemical beta-sitosterol. For example, an increase of the beta-sitosterol of at least 1 percent, particularly in a range of 17 to 20-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a sll1761-protein, or if the activity of the polypeptide Sll1761, preferably represented by SEQ ID NO. 55380, or a homolog or fragment thereof, or if the activity of a polypeptide encoded by a nucleic acid molecule comprising the nucleic acid SEQ ID NO. 55379, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 55379 or polypeptide SEQ ID NO. 55380, respectively, or a homolog or a fragment thereof, is increased or generated, or if the activity sll1761-protein is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 22 to 44-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a acyl transferase, or if the activity of the polypeptide Sll1848, preferably represented by SEQ ID NO. 86448, or a homolog or fragment thereof, or if the activity of a polypeptide encoded by a nucleic acid molecule comprising the nucleic acid SEQ ID NO. 86447, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 86447 or polypeptide SEQ ID NO. 86448, respectively, or a homolog or a fragment thereof, is increased or generated, or if the activity acyl transferase is increased or generated in a non-human organism, like a microorganism or a plant cell, plant or part thereof, especially with plastidic localization, whereby the respective line disclose in column 7 the fine chemical beta-sitosterol. For example, an increase of the beta-sitosterol of at least 1 percent, particularly in a range of 16 to 26-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a cation-transporting ATPase, or if the activity of the polypeptide Sll1920, preferably represented by SEQ ID NO. 55386, or a homolog or fragment thereof, or if the activity of a polypeptide encoded by a nucleic acid molecule comprising the nucleic acid SEQ ID NO. 55385, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 55385 or polypeptide SEQ ID NO. 55386, respectively, or a homolog or a fragment thereof, is increased or generated, or if the activity cation-transporting ATPase is increased or generated in a non-human organism, like a microorganism or a plant cell, plant or part thereof, especially with mitochondrial localization, whereby the respective line disclose in column 7 the fine chemical campesterol. For example, an increase of the campesterol of at least 1 percent, particularly in a range of 20 to 66-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a oxidoreductase, or if the activity of the polypeptide Slr0338, preferably represented by SEQ ID NO. 11991, or a homolog or fragment thereof, or if the activity of a polypeptide encoded by a nucleic acid molecule comprising the nucleic acid SEQ ID NO. 11990, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 11990 or polypeptide SEQ ID NO. 11991, respectively, or a homolog or a fragment thereof, is increased or generated, or if the activity oxidoreductase is increased or generated in a non-human organism, like a microorganism or a plant cell, plant or part thereof, especially with non-targeted localization, whereby the respective line disclose in column 7 the fine chemical beta-sitosterol. For example, an increase of the beta-sitosterol of at least 1 percent, particularly in a range of 14 to 19-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a permease protein of ABC transporter, or if the activity of the polypeptide Slr0949, preferably represented by SEQ ID NO. 119223, or a homolog or fragment thereof, or if the activity of a polypeptide encoded by a nucleic acid molecule comprising the nucleic acid SEQ ID NO. 119222, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 119222 or polypeptide SEQ ID NO. 119223, respectively, or a homolog or a fragment thereof, is increased or generated, or if the activity permease protein of ABC transporter is increased or generated in a non-human organism, like a microorganism or a plant cell, plant or part thereof, especially with mitochondrial localization, whereby the respective line disclose in column 7 the fine chemical campesterol. For example, an increase of the campesterol of at least 1 percent, particularly in a range of 16 to 28-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a tryptophan synthase alpha chain, or if the activity of the polypeptide Slr0966, preferably represented by SEQ ID NO. 87656, or a homolog or fragment thereof, or if the activity of a polypeptide encoded by a nucleic acid molecule comprising the nucleic acid SEQ ID NO. 87655, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 87655 or polypeptide SEQ ID NO. 87656, respectively, or a homolog or a fragment thereof, is increased or generated, or if the activity tryptophan synthase alpha chain is increased or generated in a non-human organism, like a microorganism or a plant cell, plant or part thereof, especially with plastidic localization, whereby the respective line disclose in column 7 the fine chemical campesterol. For example, an increase of the campesterol of at least 1 percent, particularly in a range of 18 to 20-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a multiple antibiotic resistance protein, or if the activity of the polypeptide TTC1193, preferably represented by SEQ ID NO. 61724, or a homolog or fragment thereof, or if the activity of a polypeptide encoded by a nucleic acid molecule comprising the nucleic acid SEQ ID NO. 61723, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 61723 or polypeptide SEQ ID NO. 61724, respectively, or a homolog or a fragment thereof, is increased or generated, or if the activity multiple antibiotic resistance protein is increased or generated in a non-human organism, like a microorganism or a plant cell, plant or part thereof, especially with non-targeted localization, whereby the respective line disclose in column 7 the fine chemical beta-sitosterol. For example, an increase of the beta-sitosterol of at least 1 percent, particularly in a range of 15 to 17-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a multiple antibiotic resistance protein, or if the activity of the polypeptide TTC1193, preferably represented by SEQ ID NO. 61724, or a homolog or fragment thereof, or if the activity of a polypeptide encoded by a nucleic acid molecule comprising the nucleic acid SEQ ID NO. 61723, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 61723 or polypeptide SEQ ID NO. 61724, respectively, or a homolog or a fragment thereof, is increased or generated, or if the activity multiple antibiotic resistance protein is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 17 to 39-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a TTC1386-protein, or if the activity of the polypeptide TTC1386, preferably represented by SEQ ID NO. 62080, or a homolog or fragment thereof, or if the activity of a polypeptide encoded by a nucleic acid molecule comprising the nucleic acid SEQ ID NO. 62079, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 62079 or polypeptide SEQ ID NO. 62080, respectively, or a homolog or a fragment thereof, is increased or generated, or if the activity TTC1386-protein is increased or generated in a non-human organism, like a microorganism or a plant cell, plant or part thereof, especially with non-targeted localization, whereby the respective line disclose in column 7 the fine chemical beta-sitosterol. For example, an increase of the beta-sitosterol of at least 1 percent, particularly in a range of 24 to 31-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a TTC1386-protein, or if the activity of the polypeptide TTC1386, preferably represented by SEQ ID NO. 62080, or a homolog or fragment thereof, or if the activity of a polypeptide encoded by a nucleic acid molecule comprising the nucleic acid SEQ ID NO. 62079, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 62079 or polypeptide SEQ ID NO. 62080, respectively, or a homolog or a fragment thereof, is increased or generated, or if the activity TTC1386-protein is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 34 to 50-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a homocitrate synthase, or if the activity of the polypeptide TTC1550, preferably represented by SEQ ID NO. 12975, or a homolog or fragment thereof, or if the activity of a polypeptide encoded by a nucleic acid molecule comprising the nucleic acid SEQ ID NO. 12974, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 12974 or polypeptide SEQ ID NO. 12975, respectively, or a homolog or a fragment thereof, is increased or generated, or if the activity homocitrate synthase is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 17 to 23-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a homocitrate synthase, or if the activity of the polypeptide Ydl131w, preferably represented by SEQ ID NO. 113225, or a homolog or fragment thereof, or if the activity of a polypeptide encoded by a nucleic acid molecule comprising the nucleic acid SEQ ID NO. 113224, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 113224 or polypeptide SEQ ID NO. 113225, respectively, or a homolog or a fragment thereof, is increased or generated, or if the activity homocitrate synthase is increased or generated in a non-human organism, like a microorganism or a plant cell, plant or part thereof, especially with plastidic localization, whereby the respective line disclose in column 7 the fine chemical campesterol. For example, an increase of the campesterol of at least 1 percent, particularly in a range of 18 to 98-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a cyclin, or if the activity of the polypeptide Ydl155w, preferably represented by SEQ ID NO. 14172, or a homolog or fragment thereof, or if the activity of a polypeptide encoded by a nucleic acid molecule comprising the nucleic acid SEQ ID NO. 14171, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 14171 or polypeptide SEQ ID NO. 14172, respectively, or a homolog or a fragment thereof, is increased or generated, or if the activity cyclin is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 17 to 63-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a ydl235cprotein, or if the activity of the polypeptide Ydl235c, preferably represented by SEQ ID NO. 113314, or a homolog or fragment thereof, or if the activity of a polypeptide encoded by a nucleic acid molecule comprising the nucleic acid SEQ ID NO. 113313, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 113313 or polypeptide SEQ ID NO. 113314, respectively, or a homolog or a fragment thereof, is increased or generated, or if the activity ydl235c-protein is increased or generated in a non-human organism, like a microorganism or a plant cell, plant or part thereof, especially with plastidic localization, whereby the respective line disclose in column 7 the fine chemical campesterol. For example, an increase of the campesterol of at least 1 percent, particularly in a range of 21 to 37-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a oxidoreductase, or if the activity of the polypeptide Yg1039w, preferably represented by SEQ ID NO. 120437, or a homolog or fragment thereof, or if the activity of a polypeptide encoded by a nucleic acid molecule comprising the nucleic acid SEQ ID NO. 120436, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 120436 or polypeptide SEQ ID NO. 120437, respectively, or a homolog or a fragment thereof, is increased or generated, or if the activity oxidoreductase is increased or generated in a non-human organism, like a microorganism or a plant cell, plant or part thereof, especially with plastidic localization, whereby the respective line disclose in column 7 the fine chemical campesterol. For example, an increase of the campesterol of at least 1 percent, particularly in a range of 17 to 33-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a polygalacturonase, or if the activity of the polypeptide Yjr153w, preferably represented by SEQ ID NO. 66275, or a homolog or fragment thereof, or if the activity of a polypeptide encoded by a nucleic acid molecule comprising the nucleic acid SEQ ID NO. 66274, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 66274 or polypeptide SEQ ID NO. 66275, respectively, or a homolog or a fragment thereof, is increased or generated, or if the activity polygalacturonase is increased or generated in a non-human organism, like a microorganism or a plant cell, plant or part thereof, especially with non-targeted localization, whereby the respective line disclose in column 7 the fine chemical campesterol. For example, an increase of the campesterol of at least 1 percent, particularly in a range of 16 to 42-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a ynl321w-protein, or if the activity of the polypeptide Ynl321w, preferably represented by SEQ ID NO. 129547, or a homolog or fragment thereof, or if the activity of a polypeptide encoded by a nucleic acid molecule comprising the nucleic acid SEQ ID NO. 129546, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 129546 or polypeptide SEQ ID NO. 129547, respectively, or a homolog or a fragment thereof, is increased or generated, or if the activity ynl321w-protein is increased or generated in a non-human organism, like a microorganism or a plant cell, plant or part thereof, especially with plastidic localization, whereby the respective line disclose in column 7 the fine chemical campesterol. For example, an increase of the campesterol of at least 1 percent, particularly in a range of 16 to 28-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a acetyl-CoA acetyltransferase, or if the activity of the polypeptide Ypl028w, preferably represented by SEQ ID NO. 92669, or a homolog or fragment thereof, or if the activity of a polypeptide encoded by a nucleic acid molecule comprising the nucleic acid SEQ ID NO. 92668, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 92668 or polypeptide SEQ ID NO. 92669, respectively, or a homolog or a fragment thereof, is increased or generated, or if the activity acetyl-CoA acetyltransferase is increased or generated in a non-human organism, like a microorganism or a plant cell, plant or part thereof, especially with non-targeted localization, whereby the respective line disclose in column 7 the fine chemical beta-sitosterol. For example, an increase of the beta-sitosterol of at least 1 percent, particularly in a range of 12 to 23-percent is conferred as compared to a corresponding non-transformed wild type non-human organism.

Accordingly, in one embodiment, an increase of beta-sitosterol in a non-human organism, as compared to a corresponding non-transformed wild type non-human organism, is conferred in the process of the invention, if the activity of a polypeptide showing the activity of a ypr170cprotein, or if the activity of the polypeptide Ypr170c, preferably represented by SEQ ID NO. 113648, or a homolog or fragment thereof, or if the activity of a polypeptide encoded by a nucleic acid molecule comprising the nucleic acid SEQ ID NO. 113647, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 113647 or polypeptide SEQ ID NO. 113648, respectively, or a homolog or a fragment thereof, is increased or generated, or if the activity ypr170c-protein is increased or generated in a non-human organism, like a microorganism or a plant cell, plant or part thereof, especially with plastidic localization, whereby the respective line disclose in column 7 the fine chemical beta-sitosterol. For example, an increase of the beta-sitosterol of at least 1 percent, particularly in a range of 15 to 32-percent is conferred as compared to a corresponding non-transformed wild type non-human organism.

Accordingly, in one embodiment, an increase of campesterol in a non-human organism, as compared to a corresponding non-transformed wild type non-human organism, is conferred in the process of the invention, if the activity of a polypeptide showing the activity of a glucose-6-phosphate 1-dehydrogenase, or if the activity of the polypeptide 59582753_SOYBEAN, preferably represented by SEQ ID NO. 16884, or a homolog or fragment thereof, or if the activity of a polypeptide encoded by a nucleic acid molecule comprising the nucleic acid SEQ ID NO. 16883, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 14, column 5 or 8 in the respective same line as the nucleic acid molecule SEQ ID NO. 16883 or polypeptide SEQ ID NO. 16884, respectively, or a homolog or a fragment thereof, is increased or generated, or if the activity glucose-6-phosphate 1-dehydrogenase is increased or generated in a non-human organism, like a microorganism or a plant cell, plant or part thereof, especially with plastidic localization, whereby the respective line disclose in column 7 the fine chemical campesterol. For example, an increase of the campesterol of at least 1 percent, partitularly in a range of 18 to 52-percent is conferred as compared to a corresponding non-transformed wild type non-human organism.

[0096.1.7.14] to [0103.1.7.14] for the disclosure of these paragraphs see [0096.1.7.7] to [0103.1.7.7] above

In this paragraph “A” means “Goffeau et al., Science 274 (5287), 546 (1996)”, “B” means “Blattner et al., Science 277 (5331), 1453 (1997)”, “C” means “Nakamura et al., Nucleic Acids Res. 28(1), 72 (2000)”, “D” means “deposition of the sequences to the EMBL/GenBank/DDBJ databases under “Sequencing of the draft genome assembly of Azotobacter vinelandii AvOP”. in June 2005 by Copeland et al.”, “E” means “Henne et al., Nat. Biotechnol. 22 (5), 547 (2004)”, “F” means “Kaul et al., Nature 408 (6814), 796 (2000)”, and “G” means “Matsumoto et al., Nature 436 (7052), 793 (2005)”.

The nucleic acid sequence of At1g07420 from Arabidopsis thaliana, e.g. as shown in the respective line in column 5 of Table I, application no. 14, has been published in F. And the activity of the gene product thereof is the activity of C-4 sterol methyl oxidase.

Accordingly, in one embodiment, the process of the present invention for producing campesterol in a non-human organism, like a microorganism or a plant or a part thereof, comprises increasing or generating the activity of a gene product with the activity of a gene product conferring the activity of “C-4 sterol methyl oxidase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said At1g07420, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said At1g07420, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said At1g07420, or a functional equivalent or a homolog thereof as depicted in column 8 of Table II, application no. 14, preferably a homolog or functional equivalent as depicted in column 8 of Table II B, application no. 14, and being depicted in the same respective line as said At1g07420, and preferably the activity is increased non-targeted, whereby the respective line disclose in column 7 the fine chemical campesterol.

Accordingly, in one embodiment, the molecule which activity is to be increased in the process of the invention is the gene product with an activity as a “C-4 sterol methyl oxidase”, preferably it is the molecule of section (a) or (b) of this paragraph.

In particular, it was observed that in plants, especially in Arabidopsis thaliana, increasing or generating the activity of a gene product non-targeted with the activity of a “C-4 sterol methyl oxidase”, preferably being encoded by a gene comprising the nucleic acid sequence SEQ ID NO. 131003, preferably the coding region thereof, conferred the production of or the increase in campesterol compared with the wild type control.

The nucleic acid sequence of At1g07430 from Arabidopsis thaliana, e.g. as shown in the respective line in column 5 of Table I, application no. 14, has been published in F. And the activity of the gene product thereof is the activity of protein phosphatase.

Accordingly, in one embodiment, the process of the present invention for producing campesterol in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said At1g07430, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said At1g07430, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said At1g07430, or a functional equivalent or a homolog thereof as depicted in column 8 of Table II, application no. 14, preferably a homolog or functional equivalent as depicted in column 8 of Table II B, application no. 14, and being depicted in the same respective line as said At1g07430, and preferably the activity is increased non-targeted, whereby the respective line disclose in column 7 the fine chemical campesterol.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 17451, preferably the coding region thereof, conferred the production of or the increase in campesterol compared with the wild type control.

The nucleic acid sequence of At1g36730 from Arabidopsis thaliana, e.g. as shown in the respective line in column 5 of Table I, application no. 14, has been published in F. And the activity of the gene product thereof is the activity of eukaryotic translation initiation factor 5.

Accordingly, in one embodiment, the process of the present invention for producing campesterol in a non-human organism, like a microorganism or a plant or a part thereof, comprises increasing or generating the activity of a gene product with the activity of a gene product conferring the activity of “eukaryotic translation initiation factor 5”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said At1g36730, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said At1g36730, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said At1g36730, or a functional equivalent or a homolog thereof as depicted in column 8 of Table II, application no. 14, preferably a homolog or functional equivalent as depicted in column 8 of Table II B, application no. 14, and being depicted in the same respective line as said At1g36730, and preferably the activity is increased non-targeted, whereby the respective line disclose in column 7 the fine chemical campesterol.

Accordingly, in one embodiment, the molecule which activity is to be increased in the process of the invention is the gene product with an activity as a “eukaryotic translation initiation factor 5”, preferably it is the molecule of section (a) or (b) of this paragraph.

In particular, it was observed that in plants, especially in Arabidopsis thaliana, increasing or generating the activity of a gene product non-targeted with the activity of a “eukaryotic translation initiation factor 5”, preferably being encoded by a gene comprising the nucleic acid sequence SEQ ID NO. 17968, preferably the coding region thereof, conferred the production of or the increase in campesterol compared with the wild type control.

The nucleic acid sequence of At1g61950 from Arabidopsis thaliana, e.g. as shown in the respective line in column 5 of Table I, application no. 14, has been published in F. And the activity of the gene product thereof is the activity of calcium-dependent protein kinase.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said At1g61950, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said At1g61950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said At1g61950, or a functional equivalent or a homolog thereof as depicted in column 8 of Table II, application no. 14, preferably a homolog or functional equivalent as depicted in column 8 of Table II B, application no. 14, and being depicted in the same respective line as said At1g61950, and preferably the activity is increased non-targeted, whereby the respective line disclose in column 7 the fine chemical beta-sitosterol.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 18869, preferably the coding region thereof, conferred the production of or the increase in beta-sitosterol compared with the wild type control.

The nucleic acid sequence of At1g61950 from Arabidopsis thaliana, e.g. as shown in the respective line in column 5 of Table I, application no. 14, has been published in F. And the activity of the gene product thereof is the activity of calcium-dependent protein kinase.

Accordingly, in one embodiment, the process of the present invention for producing campesterol in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said At1g61950, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said At1g61950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said At1g61950, or a functional equivalent or a homolog thereof as depicted in column 8 of Table II, application no. 14, preferably a homolog or functional equivalent as depicted in column 8 of Table II B, application no. 14, and being depicted in the same respective line as said At1g61950, and preferably the activity is increased non-targeted, whereby the respective line disclose in column 7 the fine chemical campesterol.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 18869, preferably the coding region thereof, conferred the production of or the increase in campesterol compared with the wild type control.

The nucleic acid sequence of At1g68320 from Arabidopsis thaliana, e.g. as shown in the respective line in column 5 of Table I, application no. 14, has been published in F. And the activity of the gene product thereof is the activity of transcription factor.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said At1g68320, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said At1g68320, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said At1g68320, or a functional equivalent or a homolog thereof as depicted in column 8 of Table II, application no. 14, preferably a homolog or functional equivalent as depicted in column 8 of Table II B, application no. 14, and being depicted in the same respective line as said At1g68320, and preferably the activity is increased non-targeted, whereby the respective line disclose in column 7 the fine chemical beta-sitosterol.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 1061, preferably the coding region thereof, conferred the production of or the increase in beta-sitosterol compared with the wild type control.

The nucleic acid sequence of At1g68320 from Arabidopsis thaliana, e.g. as shown in the respective line in column 5 of Table I, application no. 14, has been published in F. And the activity of the gene product thereof is the activity of transcription factor.

Accordingly, in one embodiment, the process of the present invention for producing campesterol in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said At1g68320, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said At1g68320, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said At1g68320, or a functional equivalent or a homolog thereof as depicted in column 8 of Table II, application no. 14, preferably a homolog or functional equivalent as depicted in column 8 of Table II B, application no. 14, and being depicted in the same respective line as said At1g68320, and preferably the activity is increased non-targeted, whereby the respective line disclose in column 7 the fine chemical campesterol.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 1061, preferably the coding region thereof, conferred the production of or the increase in campesterol compared with the wild type control.

The nucleic acid sequence of At1g72770 from Arabidopsis thaliana, e.g. as shown in the respective line in column 5 of Table I, application no. 14, has been published in F. And the activity of the gene product thereof is the activity of protein phosphatase.

Accordingly, in one embodiment, the process of the present invention for producing campesterol in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said At1g72770, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said At1g72770, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said At1g72770, or a functional equivalent or a homolog thereof as depicted in column 8 of Table II, application no. 14, preferably a homolog or functional equivalent as depicted in column 8 of Table II B, application no. 14, and being depicted in the same respective line as said At1g72770, and preferably the activity is increased non-targeted, whereby the respective line disclose in column 7 the fine chemical campesterol.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19419, preferably the coding region thereof, conferred the production of or the increase in campesterol compared with the wild type control.

The nucleic acid sequence of At3g04710 from Arabidopsis thaliana, e.g. as shown in the respective line in column 5 of Table I, application no. 14, has been published in F. And the activity of the gene product thereof is the activity of ankyrin repeat family protein.

Accordingly, in one embodiment, the process of the present invention for producing campesterol in a non-human organism, like a microorganism or a plant or a part thereof, comprises increasing or generating the activity of a gene product with the activity of a gene product conferring the activity of “ankyrin repeat family protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said At3g04710, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said At3g04710, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said At3g04710, or a functional equivalent or a homolog thereof as depicted in column 8 of Table II, application no. 14, preferably a homolog or functional equivalent as depicted in column 8 of Table II B, application no. 14, and being depicted in the same respective line as said At3g04710, and preferably the activity is increased non-targeted, whereby the respective line disclose in column 7 the fine chemical campesterol.

Accordingly, in one embodiment, the molecule which activity is to be increased in the process of the invention is the gene product with an activity as a “ankyrin repeat family protein”, preferably it is the molecule of section (a) or (b) of this paragraph.

In particular, it was observed that in plants, especially in Arabidopsis thaliana, increasing or generating the activity of a gene product non-targeted with the activity of a “ankyrin repeat family protein”, preferably being encoded by a gene comprising the nucleic acid sequence SEQ ID NO. 21902, preferably the coding region thereof, conferred the production of or the increase in campesterol compared with the wild type control.

The nucleic acid sequence of At4g09960 from Arabidopsis thaliana, e.g. as shown in the respective line in column 5 of Table I, application no. 14, has been published in F. And the activity of the gene product thereof is the activity of transcription factor.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said At4g09960, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said At4g09960, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said At4g09960, or a functional equivalent or a homolog thereof as depicted in column 8 of Table II, application no. 14, preferably a homolog or functional equivalent as depicted in column 8 of Table II B, application no. 14, and being depicted in the same respective line as said At4g09960, and preferably the activity is increased non-targeted, whereby the respective line disclose in column 7 the fine chemical beta-sitosterol.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 109717, preferably the coding region thereof, conferred the production of or the increase in beta-sitosterol compared with the wild type control.

The nucleic acid sequence of At4g09960 from Arabidopsis thaliana, e.g. as shown in the respective line in column 5 of Table I, application no. 14, has been published in F. And the activity of the gene product thereof is the activity of transcription factor.

Accordingly, in one embodiment, the process of the present invention for producing campesterol in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said At4g09960, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said At4g09960, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said At4g09960, or a functional equivalent or a homolog thereof as depicted in column 8 of Table II, application no. 14, preferably a homolog or functional equivalent as depicted in column 8 of Table II B, application no. 14, and being depicted in the same respective line as said At4g09960, and preferably the activity is increased non-targeted, whereby the respective line disclose in column 7 the fine chemical campesterol.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 109717, preferably the coding region thereof, conferred the production of or the increase in campesterol compared with the wild type control.

The nucleic acid sequence of At4g12110 from Arabidopsis thaliana, e.g. as shown in the respective line in column 5 of Table I, application no. 14, has been published in F. And the activity of the gene product thereof is the activity of sterol desaturase.

Accordingly, in one embodiment, the process of the present invention for producing campesterol in a non-human organism, like a microorganism or a plant or a part thereof, comprises increasing or generating the activity of a gene product with the activity of a gene product conferring the activity of “sterol desaturase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said At4g12110, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said At4g12110, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said At4g12110, or a functional equivalent or a homolog thereof as depicted in column 8 of Table II, application no. 14, preferably a homolog or functional equivalent as depicted in column 8 of Table II B, application no. 14, and being depicted in the same respective line as said At4g12110, and preferably the activity is increased non-targeted, whereby the respective line disclose in column 7 the fine chemical campesterol.

Accordingly, in one embodiment, the molecule which activity is to be increased in the process of the invention is the gene product with an activity as a “sterol desaturase”, preferably it is the molecule of section (a) or (b) of this paragraph.

In particular, it was observed that in plants, especially in Arabidopsis thaliana, increasing or generating the activity of a gene product non-targeted with the activity of a “sterol desaturase”, preferably being encoded by a gene comprising the nucleic acid sequence SEQ ID NO. 131208, preferably the coding region thereof, conferred the production of or the increase in campesterol compared with the wild type control.

The nucleic acid sequence of At4g15660 from Arabidopsis thaliana, e.g. as shown in the respective line in column 5 of Table I, application no. 14, has been published in F. And the activity of the gene product thereof is the activity of monothiol glutaredoxin.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said At4g15660, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said At4g15660, or a functional equivalent or a homolog thereof as depicted in column 8 of Table II, application no. 14, preferably a homolog or functional equivalent as depicted in column 8 of Table II B, application no. 14, and being depicted in the same respective line as said At4g15660, and preferably the activity is increased non-targeted, whereby the respective line disclose in column 7 the fine chemical beta-sitosterol.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in beta-sitosterol compared with the wild type control.

The nucleic acid sequence of At4g26080 from Arabidopsis thaliana, e.g. as shown in the respective line in column 5 of Table I, application no. 14, has been published in F. And the activity of the gene product thereof is the activity of protein phosphatase.

Accordingly, in one embodiment, the process of the present invention for producing campesterol in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said At4g26080, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said At4g26080, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said At4g26080, or a functional equivalent or a homolog thereof as depicted in column 8 of Table II, application no. 14, preferably a homolog or functional equivalent as depicted in column 8 of Table II B, application no. 14, and being depicted in the same respective line as said At4g26080, and preferably the activity is increased non-targeted, whereby the respective line disclose in column 7 the fine chemical campesterol.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 68849, preferably the coding region thereof, conferred the production of or the increase in campesterol compared with the wild type control.

The nucleic acid sequence of At4g35310 from Arabidopsis thaliana, e.g. as shown in the respective line in column 5 of Table I, application no. 14, has been published in F. And the activity of the gene product thereof is the activity of calcium-dependent protein kinase.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said At4g35310, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said At4g35310, or a functional equivalent or a homolog thereof as depicted in column 8 of Table II, application no. 14, preferably a homolog or functional equivalent as depicted in column 8 of Table II B, application no. 14, and being depicted in the same respective line as said At4g35310, and preferably the activity is increased non-targeted, whereby the respective line disclose in column 7 the fine chemical beta-sitosterol.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in beta-sitosterol compared with the wild type control.

The nucleic acid sequence of At4g35310 from Arabidopsis thaliana, e.g. as shown in the respective line in column 5 of Table I, application no. 14, has been published in F. And the activity of the gene product thereof is the activity of calcium-dependent protein kinase.

Accordingly, in one embodiment, the process of the present invention for producing campesterol in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said At4g35310, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said At4g35310, or a functional equivalent or a homolog thereof as depicted in column 8 of Table II, application no. 14, preferably a homolog or functional equivalent as depicted in column 8 of Table II B, application no. 14, and being depicted in the same respective line as said At4g35310, and preferably the activity is increased non-targeted, whereby the respective line disclose in column 7 the fine chemical campesterol.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in campesterol compared with the wild type control.

The nucleic acid sequence of At5g57050 from Arabidopsis thaliana, e.g. as shown in the respective line in column 5 of Table I, application no. 14, has been published in F. And the activity of the gene product thereof is the activity of protein phosphatase.

Accordingly, in one embodiment, the process of the present invention for producing campesterol in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said At5g57050, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said At5g57050, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said At5g57050, or a functional equivalent or a homolog thereof as depicted in column 8 of Table II, application no. 14, preferably a homolog or functional equivalent as depicted in column 8 of Table II B, application no. 14, and being depicted in the same respective line as said At5g57050, and preferably the activity is increased non-targeted, whereby the respective line disclose in column 7 the fine chemical campesterol.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 5318, preferably the coding region thereof, conferred the production of or the increase in campesterol compared with the wild type control.

The nucleic acid sequence of At5g59220 from Arabidopsis thaliana, e.g. as shown in the respective line in column 5 of Table I, application no. 14, has been published in F. And the activity of the gene product thereof is the activity of protein phosphatase.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said At5g59220, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said At5g59220, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said At5g59220, or a functional equivalent or a homolog thereof as depicted in column 8 of Table II, application no. 14, preferably a homolog or functional equivalent as depicted in column 8 of Table II B, application no. 14, and being depicted in the same respective line as said At5g59220, and preferably the activity is increased non-targeted, whereby the respective line disclose in column 7 the fine chemical beta-sitosterol.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 25498, preferably the coding region thereof, conferred the production of or the increase in beta-sitosterol compared with the wild type control.

The nucleic acid sequence of At5g66710 from Arabidopsis thaliana, e.g. as shown in the respective line in column 5 of Table I, application no. 14, has been published in F. And the activity of the gene product thereof is the activity of protein kinase.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said At5g66710, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said At5g66710, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said At5g66710, or a functional equivalent or a homolog thereof as depicted in column 8 of Table II, application no. 14, preferably a homolog or functional equivalent as depicted in column 8 of Table II B, application no. 14, and being depicted in the same respective line as said At5g66710, and preferably the activity is increased non-targeted, whereby the respective line disclose in column 7 the fine chemical beta-sitosterol.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 122803, preferably the coding region thereof, conferred the production of or the increase in beta-sitosterol compared with the wild type control.

The nucleic acid sequence of AvinDRAFT_{—}1624 from Azotobacter vinelandii, e.g. as shown in the respective line in column 5 of Table I, application no. 14, has been published in D. And the activity of the gene product thereof is the activity of sec-independent protein translocase.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “sec-independent protein translocase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said AvinDRAFT_{—}1624, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said AvinDRAFT_{—}1624, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said AvinDRAFT_{—}1624, or a functional equivalent or a homolog thereof as depicted in column 8 of Table II, application no. 14, preferably a homolog or functional equivalent as depicted in column 8 of Table II B, application no. 14, and being depicted in the same respective line as said AvinDRAFT_{—}1624, and preferably the activity is increased non-targeted, whereby the respective line disclose in column 7 the fine chemical beta-sitosterol.

Accordingly, in one embodiment, the molecule which activity is to be increased in the process of the invention is the gene product with an activity as a “sec-independent protein translocase”, preferably it is the molecule of section (a) or (b) of this paragraph.

In particular, it was observed that in plants, especially in Arabidopsis thaliana, increasing or generating the activity of a gene product non-targeted with the activity of a “sec-independent protein translocase”, preferably being encoded by a gene comprising the nucleic acid sequence SEQ ID NO. 26196, preferably the coding region thereof, conferred the production of or the increase in beta-sitosterol compared with the wild type control.

The nucleic acid sequence of AvinDRAFT_{—}3028 from Azotobacter vinelandii, e.g. as shown in the respective line in column 5 of Table I, application no. 14, has been published in D. And the activity of the gene product thereof is the activity of 2-oxoglutarate dehydrogenase E1 subunit.

Accordingly, in one embodiment, the process of the present invention for producing campesterol in a non-human organism, like a microorganism or a plant or a part thereof, comprises increasing or generating the activity of a gene product with the activity of a gene product conferring the activity of “2-oxoglutarate dehydrogenase E1 subunit”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said AvinDRAFT_{—}3028, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said AvinDRAFT_{—}3028, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said AvinDRAFT_{—}3028, or a functional equivalent or a homolog thereof as depicted in column 8 of Table II, application no. 14, preferably a homolog or functional equivalent as depicted in column 8 of Table II B, application no. 14, and being depicted in the same respective line as said AvinDRAFT_{—}3028, and preferably the activity is increased non-targeted, whereby the respective line disclose in column 7 the fine chemical campesterol.

Accordingly, in one embodiment, the molecule which activity is to be increased in the process of the invention is the gene product with an activity as a “2-oxoglutarate dehydrogenase E1 subunit”, preferably it is the molecule of section (a) or (b) of this paragraph.

In particular, it was observed that in plants, especially in Arabidopsis thaliana, increasing or generating the activity of a gene product non-targeted with the activity of a “2-oxoglutarate dehydrogenase E1 subunit”, preferably being encoded by a gene comprising the nucleic acid sequence SEQ ID NO. 6075, preferably the coding region thereof, conferred the production of or the increase in campesterol compared with the wild type control.

The nucleic acid sequence of AvinDRAFT_{—}3253 from Azotobacter vinelandii, e.g. as shown in the respective line in column 5 of Table I, application no. 14, has been published in D. And the activity of the gene product thereof is the activity of oxidoreductase.

Accordingly, in one embodiment, the process of the present invention for producing campesterol in a non-human organism, like a microorganism or a plant or a part thereof, comprises increasing or generating the activity of a gene product with the activity of a gene product conferring the activity of “oxidoreductase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said AvinDRAFT_{—}3253, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said AvinDRAFT_{—}3253, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said AvinDRAFT_{—}3253, or a functional equivalent or a homolog thereof as depicted in column 8 of Table II, application no. 14, preferably a homolog or functional equivalent as depicted in column 8 of Table II B, application no. 14, and being depicted in the same respective line as said AvinDRAFT_{—}3253, and preferably the activity is increased non-targeted, whereby the respective line disclose in column 7 the fine chemical campesterol.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 29500, preferably the coding region thereof, conferred the production of or the increase in campesterol compared with the wild type control.

The nucleic acid sequence of AvinDRAFT_{—}3605 from Azotobacter vinelandii, e.g. as shown in the respective line in column 5 of Table I, application no. 14, has been published in D. And the activity of the gene product thereof is the activity of CTP synthase.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “CTP synthase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said AvinDRAFT_{—}3605, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said AvinDRAFT_{—}3605, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said AvinDRAFT_{—}3605, or a functional equivalent or a homolog thereof as depicted in column 8 of Table II, application no. 14, preferably a homolog or functional equivalent as depicted in column 8 of Table II B, application no. 14, and being depicted in the same respective line as said AvinDRAFT_{—}3605, and preferably the activity is increased non-targeted, whereby the respective line disclose in column 7 the fine chemical beta-sitosterol.

Accordingly, in one embodiment, the molecule which activity is to be increased in the process of the invention is the gene product with an activity as a “CTP synthase”, preferably it is the molecule of section (a) or (b) of this paragraph.

In particular, it was observed that in plants, especially in Arabidopsis thaliana, increasing or generating the activity of a gene product non-targeted with the activity of a “CTP synthase”, preferably being encoded by a gene comprising the nucleic acid sequence SEQ ID NO. 31026, preferably the coding region thereof, conferred the production of or the increase in beta-sitosterol compared with the wild type control.

The nucleic acid sequence of AvinDRAFT_{—}3605 from Azotobacter vinelandii, e.g. as shown in the respective line in column 5 of Table I, application no. 14, has been published in D. And the activity of the gene product thereof is the activity of CTP synthase.

Accordingly, in one embodiment, the process of the present invention for producing campesterol in a non-human organism, like a microorganism or a plant or a part thereof, comprises increasing or generating the activity of a gene product with the activity of a gene product conferring the activity of “CTP synthase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said AvinDRAFT_{—}3605, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said AvinDRAFT_{—}3605, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said AvinDRAFT_{—}3605, or a functional equivalent or a homolog thereof as depicted in column 8 of Table II, application no. 14, preferably a homolog or functional equivalent as depicted in column 8 of Table II B, application no. 14, and being depicted in the same respective line as said AvinDRAFT_{—}3605, and preferably the activity is increased non-targeted, whereby the respective line disclose in column 7 the fine chemical campesterol.

Accordingly, in one embodiment, the molecule which activity is to be increased in the process of the invention is the gene product with an activity as a “CTP synthase”, preferably it is the molecule of section (a) or (b) of this paragraph.

In particular, it was observed that in plants, especially in Arabidopsis thaliana, increasing or generating the activity of a gene product non-targeted with the activity of a “CTP synthase”, preferably being encoded by a gene comprising the nucleic acid sequence SEQ ID NO. 31026, preferably the coding region thereof, conferred the production of or the increase in campesterol compared with the wild type control.

The nucleic acid sequence of AvinDRAFT_{—}3629 from Azotobacter vinelandii, e.g. as shown in the respective line in column 5 of Table I, application no. 14, has been published in D. And the activity of the gene product thereof is the activity of recombinase A.

Accordingly, in one embodiment, the process of the present invention for producing campesterol in a non-human organism, like a microorganism or a plant or a part thereof, comprises increasing or generating the activity of a gene product with the activity of a gene product conferring the activity of “recombinase A”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said AvinDRAFT_{—}3629, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said AvinDRAFT_{—}3629, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said AvinDRAFT_{—}3629, or a functional equivalent or a homolog thereof as depicted in column 8 of Table II, application no. 14, preferably a homolog or functional equivalent as depicted in column 8 of Table II B, application no. 14, and being depicted in the same respective line as said AvinDRAFT_{—}3629, and preferably the activity is increased non-targeted, whereby the respective line disclose in column 7 the fine chemical campesterol.

Accordingly, in one embodiment, the molecule which activity is to be increased in the process of the invention is the gene product with an activity as a “recombinase A”, preferably it is the molecule of section (a) or (b) of this paragraph.

In particular, it was observed that in plants, especially in Arabidopsis thaliana, increasing or generating the activity of a gene product non-targeted with the activity of a “recombinase A”, preferably being encoded by a gene comprising the nucleic acid sequence SEQ ID NO. 114231, preferably the coding region thereof, conferred the production of or the increase in campesterol compared with the wild type control.

The nucleic acid sequence of AvinDRAFT_{—}5103 from Azotobacter vinelandii, e.g. as shown in the respective line in column 5 of Table I, application no. 14, has been published in D. And the activity of the gene product thereof is the activity of hydrolase.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “hydrolase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said AvinDRAFT_{—}5103, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said AvinDRAFT_{—}5103, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said AvinDRAFT_{—}5103, or a functional equivalent or a homolog thereof as depicted in column 8 of Table II, application no. 14, preferably a homolog or functional equivalent as depicted in column 8 of Table II B, application no. 14, and being depicted in the same respective line as said AvinDRAFT_{—}5103, and preferably the activity is increased non-targeted, whereby the respective line disclose in column 7 the fine chemical beta-sitosterol.

Accordingly, in one embodiment, the molecule which activity is to be increased in the process of the invention is the gene product with an activity as a “hydrolase”, preferably it is the molecule of section (a) or (b) of this paragraph.

In particular, it was observed that in plants, especially in Arabidopsis thaliana, increasing or generating the activity of a gene product non-targeted with the activity of a “hydrolase”, preferably being encoded by a gene comprising the nucleic acid sequence SEQ ID NO. 6510, preferably the coding region thereof, conferred the production of or the increase in beta-sitosterol compared with the wild type control.

The nucleic acid sequence of AvinDRAFT_{—}5103 from Azotobacter vinelandii, e.g. as shown in the respective line in column 5 of Table I, application no. 14, has been published in D. And the activity of the gene product thereof is the activity of hydrolase.

Accordingly, in one embodiment, the process of the present invention for producing campesterol in a non-human organism, like a microorganism or a plant or a part thereof, comprises increasing or generating the activity of a gene product with the activity of a gene product conferring the activity of “hydrolase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said AvinDRAFT_{—}5103, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said AvinDRAFT_{—}5103, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said AvinDRAFT_{—}5103, or a functional equivalent or a homolog thereof as depicted in column 8 of Table II, application no. 14, preferably a homolog or functional equivalent as depicted in column 8 of Table II B, application no. 14, and being depicted in the same respective line as said AvinDRAFT_{—}5103, and preferably the activity is increased non-targeted, whereby the respective line disclose in column 7 the fine chemical campesterol.

Accordingly, in one embodiment, the molecule which activity is to be increased in the process of the invention is the gene product with an activity as a “hydrolase”, preferably it is the molecule of section (a) or (b) of this paragraph.

In particular, it was observed that in plants, especially in Arabidopsis thaliana, increasing or generating the activity of a gene product non-targeted with the activity of a “hydrolase”, preferably being encoded by a gene comprising the nucleic acid sequence SEQ ID NO. 6510, preferably the coding region thereof, conferred the production of or the increase in campesterol compared with the wild type control.

The nucleic acid sequence of B0004 from Escherichia coli, e.g. as shown in the respective line in column 5 of Table I, application no. 14, has been published in B. And the activity of the gene product thereof is the activity of threonine synthase.

Accordingly, in one embodiment, the process of the present invention for producing campesterol in a non-human organism, like a microorganism or a plant or a part thereof, comprises increasing or generating the activity of a gene product with the activity of a gene product conferring the activity of “threonine synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B0004, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B0004, and preferably the activity is increased plastidic, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B0004, or a functional equivalent or a homolog thereof as depicted in column 8 of Table II, application no. 14, preferably a homolog or functional equivalent as depicted in column 8 of Table II B, application no. 14, and being depicted in the same respective line as said B0004, and preferably the activity is increased plastidic, whereby the respective line disclose in column 7 the fine chemical campesterol.

Accordingly, in one embodiment, the molecule which activity is to be increased in the process of the invention is the gene product with an activity as a “threonine synthase”, preferably it is the molecule of section (a) or (b) of this paragraph.

In particular, it was observed that in plants, especially in Arabidopsis thaliana, increasing or generating the activity of a gene product plastidic with the activity of a “threonine synthase”, preferably being encoded by a gene comprising the nucleic acid sequence SEQ ID NO. 34889, preferably the coding region thereof, conferred the production of or the increase in campesterol compared with the wild type control.

The nucleic acid sequence of B0061 from Escherichia coli, e.g. as shown in the respective line in column 5 of Table I, application no. 14, has been published in B. And the activity of the gene product thereof is the activity of L-ribulose-5-phosphate 4-epimerase.

Accordingly, in one embodiment, the process of the present invention for producing campesterol in a non-human organism, like a microorganism or a plant or a part thereof, comprises increasing or generating the activity of a gene product with the activity of a gene product conferring the activity of “L-ribulose-5-phosphate 4-epimerase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B0061, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B0061, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B0061, or a functional equivalent or a homolog thereof as depicted in column 8 of Table II, application no. 14, preferably a homolog or functional equivalent as depicted in column 8 of Table II B, application no. 14, and being depicted in the same respective line as said B0061, and preferably the activity is increased non-targeted, whereby the respective line disclose in column 7 the fine chemical campesterol.

Accordingly, in one embodiment, the molecule which activity is to be increased in the process of the invention is the gene product with an activity as a “L-ribulose-5-phosphate 4-epimerase”, preferably it is the molecule of section (a) or (b) of this paragraph.

In particular, it was observed that in plants, especially in Arabidopsis thaliana, increasing or generating the activity of a gene product non-targeted with the activity of a “L-ribulose-5-phosphate 4-epimerase”, preferably being encoded by a gene comprising the nucleic acid sequence SEQ ID NO. 35204, preferably the coding region thereof, conferred the production of or the increase in campesterol compared with the wild type control.

The nucleic acid sequence of B0124 from Escherichia coli, e.g. as shown in the respective line in column 5 of Table I, application no. 14, has been published in B. And the activity of the gene product thereof is the activity of glucose dehydrogenase.

Accordingly, in one embodiment, the process of the present invention for producing campesterol in a non-human organism, like a microorganism or a plant or a part thereof, comprises increasing or generating the activity of a gene product with the activity of a gene product conferring the activity of “glucose dehydrogenase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B0124, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B0124, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B0124, or a functional equivalent or a homolog thereof as depicted in column 8 of Table II, application no. 14, preferably a homolog or functional equivalent as depicted in column 8 of Table II B, application no. 14, and being depicted in the same respective line as said B0124, and preferably the activity is increased non-targeted, whereby the respective line disclose in column 7 the fine chemical campesterol.

Accordingly, in one embodiment, the molecule which activity is to be increased in the process of the invention is the gene product with an activity as a “glucose dehydrogenase”, preferably it is the molecule of section (a) or (b) of this paragraph.

In particular, it was observed that in plants, especially in Arabidopsis thaliana, increasing or generating the activity of a gene product non-targeted with the activity of a “glucose dehydrogenase”, preferably being encoded by a gene comprising the nucleic acid sequence SEQ ID NO. 35482, preferably the coding region thereof, conferred the production of or the increase in campesterol compared with the wild type control.

The nucleic acid sequence of B0449 from Escherichia coli, e.g. as shown in the respective line in column 5 of Table I, application no. 14, has been published in B. And the activity of the gene product thereof is the activity of ATP-binding component of a transport system.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “ATP-binding component of a transport system”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said B0449, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B0449, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B0449, or a functional equivalent or a homolog thereof as depicted in column 8 of Table II, application no. 14, preferably a homolog or functional equivalent as depicted in column 8 of Table II B, application no. 14, and being depicted in the same respective line as said B0449, and preferably the activity is increased non-targeted, whereby the respective line disclose in column 7 the fine chemical beta-sitosterol.

Accordingly, in one embodiment, the molecule which activity is to be increased in the process of the invention is the gene product with an activity as a “ATP-binding component of a transport system”, preferably it is the molecule of section (a) or (b) of this paragraph.

In particular, it was observed that in plants, especially in Arabidopsis thaliana, increasing or generating the activity of a gene product non-targeted with the activity of a “ATP-binding component of a transport system”, preferably being encoded by a gene comprising the nucleic acid sequence SEQ ID NO. 7333, preferably the coding region thereof, conferred the production of or the increase in beta-sitosterol compared with the wild type control.

The nucleic acid sequence of B0449 from Escherichia coli, e.g. as shown in the respective line in column 5 of Table I, application no. 14, has been published in B. And the activity of the gene product thereof is the activity of ATP-binding component of a transport system.

Accordingly, in one embodiment, the process of the present invention for producing campesterol in a non-human organism, like a microorganism or a plant or a part thereof, comprises increasing or generating the activity of a gene product with the activity of a gene product conferring the activity of “ATP-binding component of a transport system”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B0449, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B0449, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B0449, or a functional equivalent or a homolog thereof as depicted in column 8 of Table II, application no. 14, preferably a homolog or functional equivalent as depicted in column 8 of Table II B, application no. 14, and being depicted in the same respective line as said B0449, and preferably the activity is increased non-targeted, whereby the respective line disclose in column 7 the fine chemical campesterol.

Accordingly, in one embodiment, the molecule which activity is to be increased in the process of the invention is the gene product with an activity as a “ATP-binding component of a transport system”, preferably it is the molecule of section (a) or (b) of this paragraph.

In particular, it was observed that in plants, especially in Arabidopsis thaliana, increasing or generating the activity of a gene product non-targeted with the activity of a “ATP-binding component of a transport system”, preferably being encoded by a gene comprising the nucleic acid sequence SEQ ID NO. 7333, preferably the coding region thereof, conferred the production of or the increase in campesterol compared with the wild type control.

The nucleic acid sequence of B0486 from Escherichia coli, e.g. as shown in the respective line in column 5 of Table I, application no. 14, has been published in B. And the activity of the gene product thereof is the activity of membrane transport protein.

Accordingly, in one embodiment, the process of the present invention for producing campesterol in a non-human organism, like a microorganism or a plant or a part thereof, comprises increasing or generating the activity of a gene product with the activity of a gene product conferring the activity of “membrane transport protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B0486, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B0486, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B0486, or a functional equivalent or a homolog thereof as depicted in column 8 of Table II, application no. 14, preferably a homolog or functional equivalent as depicted in column 8 of Table II B, application no. 14, and being depicted in the same respective line as said B0486, and preferably the activity is increased non-targeted, whereby the respective line disclose in column 7 the fine chemical campesterol.

Accordingly, in one embodiment, the molecule which activity is to be increased in the process of the invention is the gene product with an activity as a “membrane transport protein”, preferably it is the molecule of section (a) or (b) of this paragraph.

In particular, it was observed that in plants, especially in Arabidopsis thaliana, increasing or generating the activity of a gene product non-targeted with the activity of a “membrane transport protein”, preferably being encoded by a gene comprising the nucleic acid sequence SEQ ID NO. 7686, preferably the coding region thereof, conferred the production of or the increase in campesterol compared with the wild type control.

The nucleic acid sequence of B0856 from Escherichia coli, e.g. as shown in the respective line in column 5 of Table I, application no. 14, has been published in B. And the activity of the gene product thereof is the activity of putrescine transport system permease protein.

Accordingly, in one embodiment, the process of the present invention for producing campesterol in a non-human organism, like a microorganism or a plant or a part thereof, comprises increasing or generating the activity of a gene product with the activity of a gene product conferring the activity of “putrescine transport system permease protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B0856, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B0856, and preferably the activity is increased plastidic, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B0856, or a functional equivalent or a homolog thereof as depicted in column 8 of Table II, application no. 14, preferably a homolog or functional equivalent as depicted in column 8 of Table II B, application no. 14, and being depicted in the same respective line as said B0856, and preferably the activity is increased plastidic, whereby the respective line disclose in column 7 the fine chemical campesterol.

Accordingly, in one embodiment, the molecule which activity is to be increased in the process of the invention is the gene product with an activity as a “putrescine transport system permease protein”, preferably it is the molecule of section (a) or (b) of this paragraph.

In particular, it was observed that in plants, especially in Arabidopsis thaliana, increasing or generating the activity of a gene product plastidic with the activity of a “putrescine transport system permease protein”, preferably being encoded by a gene comprising the nucleic acid sequence SEQ ID NO. 131359, preferably the coding region thereof, conferred the production of or the increase in campesterol compared with the wild type control.

The nucleic acid sequence of B1065 from Escherichia coli, e.g. as shown in the respective line in column 5 of Table I, application no. 14, has been published in B. And the activity of the gene product thereof is the activity of multidrug resistance protein.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “multidrug resistance protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said B1065, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B1065, and preferably the activity is increased plastidic, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B1065, or a functional equivalent or a homolog thereof as depicted in column 8 of Table II, application no. 14, preferably a homolog or functional equivalent as depicted in column 8 of Table II B, application no. 14, and being depicted in the same respective line as said B1065, and preferably the activity is increased plastidic, whereby the respective line disclose in column 7 the fine chemical beta-sitosterol.

Accordingly, in one embodiment, the molecule which activity is to be increased in the process of the invention is the gene product with an activity as a “multidrug resistance protein”, preferably it is the molecule of section (a) or (b) of this paragraph.

In particular, it was observed that in plants, especially in Arabidopsis thaliana, increasing or generating the activity of a gene product plastidic with the activity of a “multidrug resistance protein”, preferably being encoded by a gene comprising the nucleic acid sequence SEQ ID NO. 76032, preferably the coding region thereof, conferred the production of or the increase in beta-sitosterol compared with the wild type control.

The nucleic acid sequence of B1186 from Escherichia coli, e.g. as shown in the respective line in column 5 of Table I, application no. 14, has been published in B. And the activity of the gene product thereof is the activity of sodium/proton antiporter.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “sodium/proton antiporter”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said B1186, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B1186, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B1186, or a functional equivalent or a homolog thereof as depicted in column 8 of Table II, application no. 14, preferably a homolog or functional equivalent as depicted in column 8 of Table II B, application no. 14, and being depicted in the same respective line as said B1186, and preferably the activity is increased non-targeted, whereby the respective line disclose in column 7 the fine chemical beta-sitosterol.

Accordingly, in one embodiment, the molecule which activity is to be increased in the process of the invention is the gene product with an activity as a “sodium/proton antiporter”, preferably it is the molecule of section (a) or (b) of this paragraph.

In particular, it was observed that in plants, especially in Arabidopsis thaliana, increasing or generating the activity of a gene product non-targeted with the activity of a “sodium/proton antiporter”, preferably being encoded by a gene comprising the nucleic acid sequence SEQ ID NO. 37400, preferably the coding region thereof, conferred the production of or the increase in beta-sitosterol compared with the wild type control.

The nucleic acid sequence of B1186 from Escherichia coli, e.g. as shown in the respective line in column 5 of Table I, application no. 14, has been published in B. And the activity of the gene product thereof is the activity of sodium/proton antiporter.

Accordingly, in one embodiment, the process of the present invention for producing campesterol in a non-human organism, like a microorganism or a plant or a part thereof, comprises increasing or generating the activity of a gene product with the activity of a gene product conferring the activity of “sodium/proton antiporter”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B1186, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B1186, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B1186, or a functional equivalent or a homolog thereof as depicted in column 8 of Table II, application no. 14, preferably a homolog or functional equivalent as depicted in column 8 of Table II B, application no. 14, and being depicted in the same respective line as said B1186, and preferably the activity is increased non-targeted, whereby the respective line disclose in column 7 the fine chemical campesterol.

Accordingly, in one embodiment, the molecule which activity is to be increased in the process of the invention is the gene product with an activity as a “sodium/proton antiporter”, preferably it is the molecule of section (a) or (b) of this paragraph.

In particular, it was observed that in plants, especially in Arabidopsis thaliana, increasing or generating the activity of a gene product non-targeted with the activity of a “sodium/proton antiporter”, preferably being encoded by a gene comprising the nucleic acid sequence SEQ ID NO. 37400, preferably the coding region thereof, conferred the production of or the increase in campesterol compared with the wild type control.

The nucleic acid sequence of B1391 from Escherichia coli, e.g. as shown in the respective line in column 5 of Table I, application no. 14, has been published in B. And the activity of the gene product thereof is the activity of phenylacetic acid degradation protein.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “phenylacetic acid degradation protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said B1391, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B1391, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B1391, or a functional equivalent or a homolog thereof as depicted in column 8 of Table II, application no. 14, preferably a homolog or functional equivalent as depicted in column 8 of Table II B, application no. 14, and being depicted in the same respective line as said B1391, and preferably the activity is increased non-targeted, whereby the respective line disclose in column 7 the fine chemical beta-sitosterol.

Accordingly, in one embodiment, the molecule which activity is to be increased in the process of the invention is the gene product with an activity as a “phenylacetic acid degradation protein”, preferably it is the molecule of section (a) or (b) of this paragraph.

In particular, it was observed that in plants, especially in Arabidopsis thaliana, increasing or generating the activity of a gene product non-targeted with the activity of a “phenylacetic acid degradation protein”, preferably being encoded by a gene comprising the nucleic acid sequence SEQ ID NO. 131489, preferably the coding region thereof, conferred the production of or the increase in beta-sitosterol compared with the wild type control.

The nucleic acid sequence of B1391 from Escherichia coli, e.g. as shown in the respective line in column 5 of Table I, application no. 14, has been published in B. And the activity of the gene product thereof is the activity of phenylacetic acid degradation protein.

Accordingly, in one embodiment, the process of the present invention for producing campesterol in a non-human organism, like a microorganism or a plant or a part thereof, comprises increasing or generating the activity of a gene product with the activity of a gene product conferring the activity of “phenylacetic acid degradation protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B1391, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B1391, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B1391, or a functional equivalent or a homolog thereof as depicted in column 8 of Table II, application no. 14, preferably a homolog or functional equivalent as depicted in column 8 of Table II B, application no. 14, and being depicted in the same respective line as said B1391, and preferably the activity is increased non-targeted, whereby the respective line disclose in column 7 the fine chemical campesterol.

Accordingly, in one embodiment, the molecule which activity is to be increased in the process of the invention is the gene product with an activity as a “phenylacetic acid degradation protein”, preferably it is the molecule of section (a) or (b) of this paragraph.

In particular, it was observed that in plants, especially in Arabidopsis thaliana, increasing or generating the activity of a gene product non-targeted with the activity of a “phenylacetic acid degradation protein”, preferably being encoded by a gene comprising the nucleic acid sequence SEQ ID NO. 131489, preferably the coding region thereof, conferred the production of or the increase in campesterol compared with the wild type control.

The nucleic acid sequence of B1401 from Escherichia coli, e.g. as shown in the respective line in column 5 of Table I, application no. 14, has been published in B. And the activity of the gene product thereof is the activity of b1401-protein.

Accordingly, in one embodiment, the process of the present invention for producing campesterol in a non-human organism, like a microorganism or a plant or a part thereof, comprises increasing or generating the activity of a gene product with the activity of a gene product conferring the activity of “b1401-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B1401, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B1401, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B1401, or a functional equivalent or a homolog thereof as depicted in column 8 of Table II, application no. 14, preferably a homolog or functional equivalent as depicted in column 8 of Table II B, application no. 14, and being depicted in the same respective line as said B1401, and preferably the activity is increased non-targeted, whereby the respective line disclose in column 7 the fine chemical campesterol.

Accordingly, in one embodiment, the molecule which activity is to be increased in the process of the invention is the gene product with an activity as a “b1401-protein”, preferably it is the molecule of section (a) or (b) of this paragraph.

In particular, it was observed that in plants, especially in Arabidopsis thaliana, increasing or generating the activity of a gene product non-targeted with the activity of a “b1401-protein”, preferably being encoded by a gene comprising the nucleic acid sequence SEQ ID NO. 131531, preferably the coding region thereof, conferred the production of or the increase in campesterol compared with the wild type control.

The nucleic acid sequence of B1431 from Escherichia coli, e.g. as shown in the respective line in column 5 of Table I, application no. 14, has been published in B. And the activity of the gene product thereof is the activity of lipoprotein precursor.

Accordingly, in one embodiment, the process of the present invention for producing campesterol in a non-human organism, like a microorganism or a plant or a part thereof, comprises increasing or generating the activity of a gene product with the activity of a gene product conferring the activity of “lipoprotein precursor”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B1431, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B1431, and preferably the activity is increased plastidic, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B1431, or a functional equivalent or a homolog thereof as depicted in column 8 of Table II, application no. 14, preferably a homolog or functional equivalent as depicted in column 8 of Table II B, application no. 14, and being depicted in the same respective line as said B1431, and preferably the activity is increased plastidic, whereby the respective line disclose in column 7 the fine chemical campesterol.

Accordingly, in one embodiment, the molecule which activity is to be increased in the process of the invention is the gene product with an activity as a “lipoprotein precursor”, preferably it is the molecule of section (a) or (b) of this paragraph.

In particular, it was observed that in plants, especially in Arabidopsis thaliana, increasing or generating the activity of a gene product plastidic with the activity of a “lipoprotein precursor”, preferably being encoded by a gene comprising the nucleic acid sequence SEQ ID NO. 38266, preferably the coding region thereof, conferred the production of or the increase in campesterol compared with the wild type control.

The nucleic acid sequence of B1597 from Escherichia coli, e.g. as shown in the respective line in column 5 of Table I, application no. 14, has been published in B. And the activity of the gene product thereof is the activity of acid shock protein.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “acid shock protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said B1597, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B1597, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B1597, or a functional equivalent or a homolog thereof as depicted in column 8 of Table II, application no. 14, preferably a homolog or functional equivalent as depicted in column 8 of Table II B, application no. 14, and being depicted in the same respective line as said B1597, and preferably the activity is increased non-targeted, whereby the respective line disclose in column 7 the fine chemical beta-sitosterol.

Accordingly, in one embodiment, the molecule which activity is to be increased in the process of the invention is the gene product with an activity as a “acid shock protein”, preferably it is the molecule of section (a) or (b) of this paragraph.

In particular, it was observed that in plants, especially in Arabidopsis thaliana, increasing or generating the activity of a gene product non-targeted with the activity of a “acid shock protein”, preferably being encoded by a gene comprising the nucleic acid sequence SEQ ID NO. 38300, preferably the coding region thereof, conferred the production of or the increase in beta-sitosterol compared with the wild type control.

The nucleic acid sequence of B1597 from Escherichia coli, e.g. as shown in the respective line in column 5 of Table I, application no. 14, has been published in B. And the activity of the gene product thereof is the activity of acid shock protein.

Accordingly, in one embodiment, the process of the present invention for producing campesterol in a non-human organism, like a microorganism or a plant or a part thereof, comprises increasing or generating the activity of a gene product with the activity of a gene product conferring the activity of “acid shock protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B1597, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B1597, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B1597, or a functional equivalent or a homolog thereof as depicted in column 8 of Table II, application no. 14, preferably a homolog or functional equivalent as depicted in column 8 of Table II B, application no. 14, and being depicted in the same respective line as said B1597, and preferably the activity is increased non-targeted, whereby the respective line disclose in column 7 the fine chemical campesterol.

Accordingly, in one embodiment, the molecule which activity is to be increased in the process of the invention is the gene product with an activity as a “acid shock protein”, preferably it is the molecule of section (a) or (b) of this paragraph.

In particular, it was observed that in plants, especially in Arabidopsis thaliana, increasing or generating the activity of a gene product non-targeted with the activity of a “acid shock protein”, preferably being encoded by a gene comprising the nucleic acid sequence SEQ ID NO. 38300, preferably the coding region thereof, conferred the production of or the increase in campesterol compared with the wild type control.

The nucleic acid sequence of B1795 from Escherichia coli, e.g. as shown in the respective line in column 5 of Table I, application no. 14, has been published in B. And the activity of the gene product thereof is the activity of B1795-protein.

Accordingly, in one embodiment, the process of the present invention for producing campesterol in a non-human organism, like a microorganism or a plant or a part thereof, comprises increasing or generating the activity of a gene product with the activity of a gene product conferring the activity of “B1795-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B1795, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B1795, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B1795, or a functional equivalent or a homolog thereof as depicted in column 8 of Table II, application no. 14, preferably a homolog or functional equivalent as depicted in column 8 of Table II B, application no. 14, and being depicted in the same respective line as said B1795, and preferably the activity is increased non-targeted, whereby the respective line disclose in column 7 the fine chemical campesterol.

Accordingly, in one embodiment, the molecule which activity is to be increased in the process of the invention is the gene product with an activity as a “B1795-protein”, preferably it is the molecule of section (a) or (b) of this paragraph.

In particular, it was observed that in plants, especially in Arabidopsis thaliana, increasing or generating the activity of a gene product non-targeted with the activity of a “B1795-protein”, preferably being encoded by a gene comprising the nucleic acid sequence SEQ ID NO. 131539, preferably the coding region thereof, conferred the production of or the increase in campesterol compared with the wild type control.

The nucleic acid sequence of B1932 from Escherichia coli, e.g. as shown in the respective line in column 5 of Table I, application no. 14, has been published in B. And the activity of the gene product thereof is the activity of acetyltransferase.

Accordingly, in one embodiment, the process of the present invention for producing campesterol in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B1932, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B1932, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B1932, or a functional equivalent or a homolog thereof as depicted in column 8 of Table II, application no. 14, preferably a homolog or functional equivalent as depicted in column 8 of Table II B, application no. 14, and being depicted in the same respective line as said B1932, and preferably the activity is increased non-targeted, whereby the respective line disclose in column 7 the fine chemical campesterol.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 131799, preferably the coding region thereof, conferred the production of or the increase in campesterol compared with the wild type control.

The nucleic acid sequence of B2107 from Escherichia coli, e.g. as shown in the respective line in column 5 of Table I, application no. 14, has been published in B. And the activity of the gene product thereof is the activity of b2107-protein.

Accordingly, in one embodiment, the process of the present invention for producing campesterol in a non-human organism, like a microorganism or a plant or a part thereof, comprises increasing or generating the activity of a gene product with the activity of a gene product conferring the activity of “b2107-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B2107, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B2107, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B2107, or a functional equivalent or a homolog thereof as depicted in column 8 of Table II, application no. 14, preferably a homolog or functional equivalent as depicted in column 8 of Table II B, application no. 14, and being depicted in the same respective line as said B2107, and preferably the activity is increased non-targeted, whereby the respective line disclose in column 7 the fine chemical campesterol.

Accordingly, in one embodiment, the molecule which activity is to be increased in the process of the invention is the gene product with an activity as a “b2107-protein”, preferably it is the molecule of section (a) or (b) of this paragraph.

In particular, it was observed that in plants, especially in Arabidopsis thaliana, increasing or generating the activity of a gene product non-targeted with the activity of a “b2107-protein”, preferably being encoded by a gene comprising the nucleic acid sequence SEQ ID NO. 39002, preferably the coding region thereof, conferred the production of or the increase in campesterol compared with the wild type control.

The nucleic acid sequence of B2276 from Escherichia coli, e.g. as shown in the respective line in column 5 of Table I, application no. 14, has been published in B. And the activity of the gene product thereof is the activity of NADH dehydrogenase I chain N.

Accordingly, in one embodiment, the process of the present invention for producing campesterol in a non-human organism, like a microorganism or a plant or a part thereof, comprises increasing or generating the activity of a gene product with the activity of a gene product conferring the activity of “NADH dehydrogenase I chain N”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B2276, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B2276, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B2276, or a functional equivalent or a homolog thereof as depicted in column 8 of Table II, application no. 14, preferably a homolog or functional equivalent as depicted in column 8 of Table II B, application no. 14, and being depicted in the same respective line as said B2276, and preferably the activity is increased non-targeted, whereby the respective line disclose in column 7 the fine chemical campesterol.

Accordingly, in one embodiment, the molecule which activity is to be increased in the process of the invention is the gene product with an activity as a “NADH dehydrogenase I chain N”, preferably it is the molecule of section (a) or (b) of this paragraph.

In particular, it was observed that in plants, especially in Arabidopsis thaliana, increasing or generating the activity of a gene product non-targeted with the activity of a “NADH dehydrogenase I chain N”, preferably being encoded by a gene comprising the nucleic acid sequence SEQ ID NO. 131839, preferably the coding region thereof, conferred the production of or the increase in campesterol compared with the wild type control.

The nucleic acid sequence of B2312 from Escherichia coli, e.g. as shown in the respective line in column 5 of Table I, application no. 14, has been published in B. And the activity of the gene product thereof is the activity of amidophosphoribosyltransferase.

Accordingly, in one embodiment, the process of the present invention for producing campesterol in a non-human organism, like a microorganism or a plant or a part thereof, comprises increasing or generating the activity of a gene product with the activity of a gene product conferring the activity of “amidophosphoribosyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B2312, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B2312, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B2312, or a functional equivalent or a homolog thereof as depicted in column 8 of Table II, application no. 14, preferably a homolog or functional equivalent as depicted in column 8 of Table II B, application no. 14, and being depicted in the same respective line as said B2312, and preferably the activity is increased non-targeted, whereby the respective line disclose in column 7 the fine chemical campesterol.

Accordingly, in one embodiment, the molecule which activity is to be increased in the process of the invention is the gene product with an activity as a “amidophosphoribosyltransferase”, preferably it is the molecule of section (a) or (b) of this paragraph.

In particular, it was observed that in plants, especially in Arabidopsis thaliana, increasing or generating the activity of a gene product non-targeted with the activity of a “amidophosphoribosyltransferase”, preferably being encoded by a gene comprising the nucleic acid sequence SEQ ID NO. 132086, preferably the coding region thereof, conferred the production of or the increase in campesterol compared with the wild type control.

The nucleic acid sequence of B2376 from Escherichia coli, e.g. as shown in the respective line in column 5 of Table I, application no. 14, has been published in B. And the activity of the gene product thereof is the activity of b2376-protein.

Accordingly, in one embodiment, the process of the present invention for producing campesterol in a non-human organism, like a microorganism or a plant or a part thereof, comprises increasing or generating the activity of a gene product with the activity of a gene product conferring the activity of “b2376-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B2376, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B2376, and preferably the activity is increased plastidic, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B2376, or a functional equivalent or a homolog thereof as depicted in column 8 of Table II, application no. 14, preferably a homolog or functional equivalent as depicted in column 8 of Table II B, application no. 14, and being depicted in the same respective line as said B2376, and preferably the activity is increased plastidic, whereby the respective line disclose in column 7 the fine chemical campesterol.

Accordingly, in one embodiment, the molecule which activity is to be increased in the process of the invention is the gene product with an activity as a “b2376-protein”, preferably it is the molecule of section (a) or (b) of this paragraph.

In particular, it was observed that in plants, especially in Arabidopsis thaliana, increasing or generating the activity of a gene product plastidic with the activity of a “b2376-protein”, preferably being encoded by a gene comprising the nucleic acid sequence SEQ ID NO. 132695, preferably the coding region thereof, conferred the production of or the increase in campesterol compared with the wild type control.

The nucleic acid sequence of B2399 from Escherichia coli, e.g. as shown in the respective line in column 5 of Table I, application no. 14, has been published in B. And the activity of the gene product thereof is the activity of b2399-protein.

Accordingly, in one embodiment, the process of the present invention for producing campesterol in a non-human organism, like a microorganism or a plant or a part thereof, comprises increasing or generating the activity of a gene product with the activity of a gene product conferring the activity of “b2399-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B2399, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B2399, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B2399, or a functional equivalent or a homolog thereof as depicted in column 8 of Table II, application no. 14, preferably a homolog or functional equivalent as depicted in column 8 of Table II B, application no. 14, and being depicted in the same respective line as said B2399, and preferably the activity is increased non-targeted, whereby the respective line disclose in column 7 the fine chemical campesterol.

Accordingly, in one embodiment, the molecule which activity is to be increased in the process of the invention is the gene product with an activity as a “b2399-protein”, preferably it is the molecule of section (a) or (b) of this paragraph.

In particular, it was observed that in plants, especially in Arabidopsis thaliana, increasing or generating the activity of a gene product non-targeted with the activity of a “b2399-protein”, preferably being encoded by a gene comprising the nucleic acid sequence SEQ ID NO. 39237, preferably the coding region thereof, conferred the production of or the increase in campesterol compared with the wild type control.

The nucleic acid sequence of B2474 from Escherichia coli, e.g. as shown in the respective line in column 5 of Table I, application no. 14, has been published in B. And the activity of the gene product thereof is the activity of b2474-protein.

Accordingly, in one embodiment, the process of the present invention for producing campesterol in a non-human organism, like a microorganism or a plant or a part thereof, comprises increasing or generating the activity of a gene product with the activity of a gene product conferring the activity of “b2474-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B2474, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B2474, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B2474, or a functional equivalent or a homolog thereof as depicted in column 8 of Table II, application no. 14, preferably a homolog or functional equivalent as depicted in column 8 of Table II B, application no. 14, and being depicted in the same respective line as said B2474, and preferably the activity is increased non-targeted, whereby the respective line disclose in column 7 the fine chemical campesterol.

Accordingly, in one embodiment, the molecule which activity is to be increased in the process of the invention is the gene product with an activity as a “b2474-protein”, preferably it is the molecule of section (a) or (b) of this paragraph.

In particular, it was observed that in plants, especially in Arabidopsis thaliana, increasing or generating the activity of a gene product non-targeted with the activity of a “b2474-protein”, preferably being encoded by a gene comprising the nucleic acid sequence SEQ ID NO. 40329, preferably the coding region thereof, conferred the production of or the increase in campesterol compared with the wild type control.

The nucleic acid sequence of B2493 from Escherichia coli, e.g. as shown in the respective line in column 5 of Table I, application no. 14, has been published in B. And the activity of the gene product thereof is the activity of permease protein.

Accordingly, in one embodiment, the process of the present invention for producing campesterol in a non-human organism, like a microorganism or a plant or a part thereof, comprises increasing or generating the activity of a gene product with the activity of a gene product conferring the activity of “permease protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B2493, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B2493, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B2493, or a functional equivalent or a homolog thereof as depicted in column 8 of Table II, application no. 14, preferably a homolog or functional equivalent as depicted in column 8 of Table II B, application no. 14, and being depicted in the same respective line as said B2493, and preferably the activity is increased non-targeted, whereby the respective line disclose in column 7 the fine chemical campesterol.

Accordingly, in one embodiment, the molecule which activity is to be increased in the process of the invention is the gene product with an activity as a “permease protein”, preferably it is the molecule of section (a) or (b) of this paragraph.

In particular, it was observed that in plants, especially in Arabidopsis thaliana, increasing or generating the activity of a gene product non-targeted with the activity of a “permease protein”, preferably being encoded by a gene comprising the nucleic acid sequence SEQ ID NO. 132706, preferably the coding region thereof, conferred the production of or the increase in campesterol compared with the wild type control.

The nucleic acid sequence of B2513 from Escherichia coli, e.g. as shown in the respective line in column 5 of Table I, application no. 14, has been published in B. And the activity of the gene product thereof is the activity of b2513-protein.

Accordingly, in one embodiment, the process of the present invention for producing campesterol in a non-human organism, like a microorganism or a plant or a part thereof, comprises increasing or generating the activity of a gene product with the activity of a gene product conferring the activity of “b2513-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B2513, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B2513, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B2513, or a functional equivalent or a homolog thereof as depicted in column 8 of Table II, application no. 14, preferably a homolog or functional equivalent as depicted in column 8 of Table II B, application no. 14, and being depicted in the same respective line as said B2513, and preferably the activity is increased non-targeted, whereby the respective line disclose in column 7 the fine chemical campesterol.

Accordingly, in one embodiment, the molecule which activity is to be increased in the process of the invention is the gene product with an activity as a “b2513-protein”, preferably it is the molecule of section (a) or (b) of this paragraph.

In particular, it was observed that in plants, especially in Arabidopsis thaliana, increasing or generating the activity of a gene product non-targeted with the activity of a “b2513-protein”, preferably being encoded by a gene comprising the nucleic acid sequence SEQ ID NO. 9167, preferably the coding region thereof, conferred the production of or the increase in campesterol compared with the wild type control.

The nucleic acid sequence of B2546 from Escherichia coli, e.g. as shown in the respective line in column 5 of Table I, application no. 14, has been published in B. And the activity of the gene product thereof is the activity of putative transport system permease protein.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “putative transport system permease protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said B2546, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B2546, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B2546, or a functional equivalent or a homolog thereof as depicted in column 8 of Table II, application no. 14, preferably a homolog or functional equivalent as depicted in column 8 of Table II B, application no. 14, and being depicted in the same respective line as said B2546, and preferably the activity is increased non-targeted, whereby the respective line disclose in column 7 the fine chemical beta-sitosterol.

Accordingly, in one embodiment, the molecule which activity is to be increased in the process of the invention is the gene product with an activity as a “putative transport system permease protein”, preferably it is the molecule of section (a) or (b) of this paragraph.

In particular, it was observed that in plants, especially in Arabidopsis thaliana, increasing or generating the activity of a gene product non-targeted with the activity of a “putative transport system permease protein”, preferably being encoded by a gene comprising the nucleic acid sequence SEQ ID NO. 99898, preferably the coding region thereof, conferred the production of or the increase in beta-sitosterol compared with the wild type control.

The nucleic acid sequence of B2546 from Escherichia coli, e.g. as shown in the respective line in column 5 of Table I, application no. 14, has been published in B. And the activity of the gene product thereof is the activity of putative transport system permease protein.

Accordingly, in one embodiment, the process of the present invention for producing campesterol in a non-human organism, like a microorganism or a plant or a part thereof, comprises increasing or generating the activity of a gene product with the activity of a gene product conferring the activity of “putative transport system permease protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B2546, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B2546, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B2546, or a functional equivalent or a homolog thereof as depicted in column 8 of Table II, application no. 14, preferably a homolog or functional equivalent as depicted in column 8 of Table II B, application no. 14, and being depicted in the same respective line as said B2546, and preferably the activity is increased non-targeted, whereby the respective line disclose in column 7 the fine chemical campesterol.

Accordingly, in one embodiment, the molecule which activity is to be increased in the process of the invention is the gene product with an activity as a “putative transport system permease protein”, preferably it is the molecule of section (a) or (b) of this paragraph.

In particular, it was observed that in plants, especially in Arabidopsis thaliana, increasing or generating the activity of a gene product non-targeted with the activity of a “putative transport system permease protein”, preferably being encoded by a gene comprising the nucleic acid sequence SEQ ID NO. 99898, preferably the coding region thereof, conferred the production of or the increase in campesterol compared with the wild type control.

The nucleic acid sequence of B2654 from Escherichia coli, e.g. as shown in the respective line in column 5 of Table I, application no. 14, has been published in B. And the activity of the gene product thereof is the activity of b2654-protein.

Accordingly, in one embodiment, the process of the present invention for producing campesterol in a non-human organism, like a microorganism or a plant or a part thereof, comprises increasing or generating the activity of a gene product with the activity of a gene product conferring the activity of “b2654-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B2654, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B2654, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B2654, or a functional equivalent or a homolog thereof as depicted in column 8 of Table II, application no. 14, preferably a homolog or functional equivalent as depicted in column 8 of Table II B, application no. 14, and being depicted in the same respective line as said B2654, and preferably the activity is increased non-targeted, whereby the respective line disclose in column 7 the fine chemical campesterol.

Accordingly, in one embodiment, the molecule which activity is to be increased in the process of the invention is the gene product with an activity as a “b2654-protein”, preferably it is the molecule of section (a) or (b) of this paragraph.

In particular, it was observed that in plants, especially in Arabidopsis thaliana, increasing or generating the activity of a gene product non-targeted with the activity of a “b2654-protein”, preferably being encoded by a gene comprising the nucleic acid sequence SEQ ID NO. 125226, preferably the coding region thereof, conferred the production of or the increase in campesterol compared with the wild type control.

The nucleic acid sequence of B2701 from Escherichia coli, e.g. as shown in the respective line in column 5 of Table I, application no. 14, has been published in B. And the activity of the gene product thereof is the activity of murein transglycosylase.

Accordingly, in one embodiment, the process of the present invention for producing campesterol in a non-human organism, like a microorganism or a plant or a part thereof, comprises increasing or generating the activity of a gene product with the activity of a gene product conferring the activity of “murein transglycosylase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B2701, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B2701, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B2701, or a functional equivalent or a homolog thereof as depicted in column 8 of Table II, application no. 14, preferably a homolog or functional equivalent as depicted in column 8 of Table II B, application no. 14, and being depicted in the same respective line as said B2701, and preferably the activity is increased non-targeted, whereby the respective line disclose in column 7 the fine chemical campesterol.

Accordingly, in one embodiment, the molecule which activity is to be increased in the process of the invention is the gene product with an activity as a “murein transglycosylase”, preferably it is the molecule of section (a) or (b) of this paragraph.

In particular, it was observed that in plants, especially in Arabidopsis thaliana, increasing or generating the activity of a gene product non-targeted with the activity of a “murein transglycosylase”, preferably being encoded by a gene comprising the nucleic acid sequence SEQ ID NO. 80756, preferably the coding region thereof, conferred the production of or the increase in campesterol compared with the wild type control.

The nucleic acid sequence of B2766 from Escherichia coli, e.g. as shown in the respective line in column 5 of Table I, application no. 14, has been published in B. And the activity of the gene product thereof is the activity of oxidoreductase.

Accordingly, in one embodiment, the process of the present invention for producing campesterol in a non-human organism, like a microorganism or a plant or a part thereof, comprises increasing or generating the activity of a gene product with the activity of a gene product conferring the activity of “oxidoreductase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B2766, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B2766, and preferably the activity is increased plastidic, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B2766, or a functional equivalent or a homolog thereof as depicted in column 8 of Table II, application no. 14, preferably a homolog or functional equivalent as depicted in column 8 of Table II B, application no. 14, and being depicted in the same respective line as said B2766, and preferably the activity is increased plastidic, whereby the respective line disclose in column 7 the fine chemical campesterol.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product plastidic with the activity of a “oxidoreductase”, preferably being encoded by a gene comprising the nucleic acid sequence SEQ ID NO. 132821, preferably the coding region thereof, conferred the production of or the increase in campesterol compared with the wild type control.

The nucleic acid sequence of B2866 from Escherichia coli, e.g. as shown in the respective line in column 5 of Table I, application no. 14, has been published in B. And the activity of the gene product thereof is the activity of xanthine dehydrogenase.

Accordingly, in one embodiment, the process of the present invention for producing campesterol in a non-human organism, like a microorganism or a plant or a part thereof, comprises increasing or generating the activity of a gene product with the activity of a gene product conferring the activity of “xanthine dehydrogenase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B2866, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B2866, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B2866, or a functional equivalent or a homolog thereof as depicted in column 8 of Table II, application no. 14, preferably a homolog or functional equivalent as depicted in column 8 of Table II B, application no. 14, and being depicted in the same respective line as said B2866, and preferably the activity is increased non-targeted, whereby the respective line disclose in column 7 the fine chemical campesterol.

Accordingly, in one embodiment, the molecule which activity is to be increased in the process of the invention is the gene product with an activity as a “xanthine dehydrogenase”, preferably it is the molecule of section (a) or (b) of this paragraph.

In particular, it was observed that in plants, especially in Arabidopsis thaliana, increasing or generating the activity of a gene product non-targeted with the activity of a “xanthine dehydrogenase”, preferably being encoded by a gene comprising the nucleic acid sequence SEQ ID NO. 115864, preferably the coding region thereof, conferred the production of or the increase in campesterol compared with the wild type control.

The nucleic acid sequence of B3151 from Escherichia coli, e.g. as shown in the respective line in column 5 of Table I, application no. 14, has been published in B. And the activity of the gene product thereof is the activity of b3151-protein.

Accordingly, in one embodiment, the process of the present invention for producing campesterol in a non-human organism, like a microorganism or a plant or a part thereof, comprises increasing or generating the activity of a gene product with the activity of a gene product conferring the activity of “b3151-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B3151, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B3151, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B3151, or a functional equivalent or a homolog thereof as depicted in column 8 of Table II, application no. 14, preferably a homolog or functional equivalent as depicted in column 8 of Table II B, application no. 14, and being depicted in the same respective line as said B3151, and preferably the activity is increased non-targeted, whereby the respective line disclose in column 7 the fine chemical campesterol.

Accordingly, in one embodiment, the molecule which activity is to be increased in the process of the invention is the gene product with an activity as a “b3151-protein”, preferably it is the molecule of section (a) or (b) of this paragraph.

In particular, it was observed that in plants, especially in Arabidopsis thaliana, increasing or generating the activity of a gene product non-targeted with the activity of a “b3151-protein”, preferably being encoded by a gene comprising the nucleic acid sequence SEQ ID NO. 42477, preferably the coding region thereof, conferred the production of or the increase in campesterol compared with the wild type control.

The nucleic acid sequence of B3262 from Escherichia coli, e.g. as shown in the respective line in column 5 of Table I, application no. 14, has been published in B. And the activity of the gene product thereof is the activity of methyltransferase.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “methyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said B3262, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B3262, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B3262, or a functional equivalent or a homolog thereof as depicted in column 8 of Table II, application no. 14, preferably a homolog or functional equivalent as depicted in column 8 of Table II B, application no. 14, and being depicted in the same respective line as said B3262, and preferably the activity is increased non-targeted, whereby the respective line disclose in column 7 the fine chemical beta-sitosterol.

Accordingly, in one embodiment, the molecule which activity is to be increased in the process of the invention is the gene product with an activity as a “methyltransferase”, preferably it is the molecule of section (a) or (b) of this paragraph.

In particular, it was observed that in plants, especially in Arabidopsis thaliana, increasing or generating the activity of a gene product non-targeted with the activity of a “methyltransferase”, preferably being encoded by a gene comprising the nucleic acid sequence SEQ ID NO. 42502, preferably the coding region thereof, conferred the production of or the increase in beta-sitosterol compared with the wild type control.

The nucleic acid sequence of B3616 from Escherichia coli, e.g. as shown in the respective line in column 5 of Table I, application no. 14, has been published in B. And the activity of the gene product thereof is the activity of threonine dehydrogenase.

Accordingly, in one embodiment, the process of the present invention for producing campesterol in a non-human organism, like a microorganism or a plant or a part thereof, comprises increasing or generating the activity of a gene product with the activity of a gene product conferring the activity of “threonine dehydrogenase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B3616, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B3616, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B3616, or a functional equivalent or a homolog thereof as depicted in column 8 of Table II, application no. 14, preferably a homolog or functional equivalent as depicted in column 8 of Table II B, application no. 14, and being depicted in the same respective line as said B3616, and preferably the activity is increased non-targeted, whereby the respective line disclose in column 7 the fine chemical campesterol.

Accordingly, in one embodiment, the molecule which activity is to be increased in the process of the invention is the gene product with an activity as a “threonine dehydrogenase”, preferably it is the molecule of section (a) or (b) of this paragraph.

In particular, it was observed that in plants, especially in Arabidopsis thaliana, increasing or generating the activity of a gene product non-targeted with the activity of a “threonine dehydrogenase”, preferably being encoded by a gene comprising the nucleic acid sequence SEQ ID NO. 42600, preferably the coding region thereof, conferred the production of or the increase in campesterol compared with the wild type control.

The nucleic acid sequence of B3989 from Escherichia coli, e.g. as shown in the respective line in column 5 of Table I, application no. 14, has been published in B. And the activity of the gene product thereof is the activity of b3989-protein.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “b3989-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said B3989, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B3989, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B3989, or a functional equivalent or a homolog thereof as depicted in column 8 of Table II, application no. 14, preferably a homolog or functional equivalent as depicted in column 8 of Table II B, application no. 14, and being depicted in the same respective line as said B3989, and preferably the activity is increased non-targeted, whereby the respective line disclose in column 7 the fine chemical beta-sitosterol.

Accordingly, in one embodiment, the molecule which activity is to be increased in the process of the invention is the gene product with an activity as a “b3989-protein”, preferably it is the molecule of section (a) or (b) of this paragraph.

In particular, it was observed that in plants, especially in Arabidopsis thaliana, increasing or generating the activity of a gene product non-targeted with the activity of a “b3989-protein”, preferably being encoded by a gene comprising the nucleic acid sequence SEQ ID NO. 44372, preferably the coding region thereof, conferred the production of or the increase in beta-sitosterol compared with the wild type control.

The nucleic acid sequence of B3989 from Escherichia coli, e.g. as shown in the respective line in column 5 of Table I, application no. 14, has been published in B. And the activity of the gene product thereof is the activity of b3989-protein.

Accordingly, in one embodiment, the process of the present invention for producing campesterol in a non-human organism, like a microorganism or a plant or a part thereof, comprises increasing or generating the activity of a gene product with the activity of a gene product conferring the activity of “b3989-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said B3989, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B3989, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B3989, or a functional equivalent or a homolog thereof as depicted in column 8 of Table II, application no. 14, preferably a homolog or functional equivalent as depicted in column 8 of Table II B, application no. 14, and being depicted in the same respective line as said B3989, and preferably the activity is increased non-targeted, whereby the respective line disclose in column 7 the fine chemical campesterol.

Accordingly, in one embodiment, the molecule which activity is to be increased in the process of the invention is the gene product with an activity as a “b3989-protein”, preferably it is the molecule of section (a) or (b) of this paragraph.

In particular, it was observed that in plants, especially in Arabidopsis thaliana, increasing or generating the activity of a gene product non-targeted with the activity of a “b3989-protein”, preferably being encoded by a gene comprising the nucleic acid sequence SEQ ID NO. 44372, preferably the coding region thereof, conferred the production of or the increase in campesterol compared with the wild type control.

The nucleic acid sequence of B4029 from Escherichia coli, e.g. as shown in the respective line in column 5 of Table I, application no. 14, has been published in B. And the activity of the gene product thereof is the activity of b4029-protein.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “b4029-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said B4029, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B4029, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B4029, or a functional equivalent or a homolog thereof as depicted in column 8 of Table II, application no. 14, preferably a homolog or functional equivalent as depicted in column 8 of Table II B, application no. 14, and being depicted in the same respective line as said B4029, and preferably the activity is increased non-targeted, whereby the respective line disclose in column 7 the fine chemical beta-sitosterol.

Accordingly, in one embodiment, the molecule which activity is to be increased in the process of the invention is the gene product with an activity as a “b4029-protein”, preferably it is the molecule of section (a) or (b) of this paragraph.

In particular, it was observed that in plants, especially in Arabidopsis thaliana, increasing or generating the activity of a gene product non-targeted with the activity of a “b4029-protein”, preferably being encoded by a gene comprising the nucleic acid sequence SEQ ID NO. 10740, preferably the coding region thereof, conferred the production of or the increase in beta-sitosterol compared with the wild type control.

The nucleic acid sequence of B4029 from Escherichia coli, e.g. as shown in the respective line in column 5 of Table I, application no. 14, has been published in B. And the activity of the gene product thereof is the activity of b4029-protein.

Accordingly, in one embodiment, the process of the present invention for producing stigmasterol in a non-human organism, like a microorganism or a plant or a part thereof, comprises increasing or generating the activity of a gene product with the activity of a gene product conferring the activity of “b4029-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical stigmasterol), application no. 14, preferably the coding region thereof, or a homolog or a fragment thereof, and being depicted in the same respective line as said B4029, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B4029, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B4029, or a functional equivalent or a homolog thereof as depicted in column 8 of Table II, application no. 14, preferably a homolog or functional equivalent as depicted in column 8 of Table II B, application no. 14, and being depicted in the same respective line as said B4029, and preferably the activity is increased non-targeted, whereby the respective line disclose in column 7 the fine chemical stigmasterol.

Accordingly, in one embodiment, the molecule which activity is to be increased in the process of the invention is the gene product with an activity as a “b4029-protein”, preferably it is the molecule of section (a) or (b) of this paragraph.

In particular, it was observed that in plants, especially in Arabidopsis thaliana, increasing or generating the activity of a gene product non-targeted with the activity of a “b4029-protein”, preferably being encoded by a gene comprising the nucleic acid sequence SEQ ID NO. 10740, preferably the coding region thereof, conferred the production of or the increase in stigmasterol compared with the wild type control.

The nucleic acid sequence of B4205 from Escherichia coli, e.g. as shown in the respective line in column 5 of Table I, application no. 14, has been published in B. And the activity of the gene product thereof is the activity of b4205-protein.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “b4205-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said B4205, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said B4205, and preferably the activity is increased plastidic, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said B4205, or a functional equivalent or a homolog thereof as depicted in column 8 of Table II, application no. 14, preferably a homolog or functional equivalent as depicted in column 8 of Table II B, application no. 14, and being depicted in the same respective line as said B4205, and preferably the activity is increased plastidic, whereby the respective line disclose in column 7 the fine chemical beta-sitosterol.

Accordingly, in one embodiment, the molecule which activity is to be increased in the process of the invention is the gene product with an activity as a “b4205-protein”, preferably it is the molecule of section (a) or (b) of this paragraph.

In particular, it was observed that in plants, especially in Arabidopsis thaliana, increasing or generating the activity of a gene product plastidic with the activity of a “b4205-protein”, preferably being encoded by a gene comprising the nucleic acid sequence SEQ ID NO. 126975, preferably the coding region thereof, conferred the production of or the increase in beta-sitosterol compared with the wild type control.

The nucleic acid sequence of GM02LC12622 from Glycine max, e.g. as shown in the respective line in column 5 of Table I, application no. 14, is unpublished. And the activity of the gene product thereof is the activity of glutaredoxin.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “glutaredoxin”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said GM02LC12622, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said GM02LC12622, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said GM02LC12622, or a functional equivalent or a homolog thereof as depicted in column 8 of Table II, application no. 14, preferably a homolog or functional equivalent as depicted in column 8 of Table II B, application no. 14, and being depicted in the same respective line as said GM02LC12622, and preferably the activity is increased non-targeted, whereby the respective line disclose in column 7 the fine chemical beta-sitosterol.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 10811, preferably the coding region thereof, conferred the production of or the increase in beta-sitosterol compared with the wild type control.

The nucleic acid sequence of GM02LC12622 from Glycine max, e.g. as shown in the respective line in column 5 of Table I, application no. 14, is unpublished. And the activity of the gene product thereof is the activity of glutaredoxin.

Accordingly, in one embodiment, the process of the present invention for producing campesterol in a non-human organism, like a microorganism or a plant or a part thereof, comprises increasing or generating the activity of a gene product with the activity of a gene product conferring the activity of “glutaredoxin”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said GM02LC12622, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said GM02LC12622, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said GM02LC12622, or a functional equivalent or a homolog thereof as depicted in column 8 of Table II, application no. 14, preferably a homolog or functional equivalent as depicted in column 8 of Table II B, application no. 14, and being depicted in the same respective line as said GM02LC12622, and preferably the activity is increased non-targeted, whereby the respective line disclose in column 7 the fine chemical campesterol.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 10811, preferably the coding region thereof, conferred the production of or the increase in campesterol compared with the wild type control.

The nucleic acid sequence of GM02LC19289 from Glycine max, e.g. as shown in the respective line in column 5 of Table I, application no. 14, is unpublished. And the activity of the gene product thereof is the activity of transcription factor.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said GM02LC19289, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said GM02LC19289, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said GM02LC19289, or a functional equivalent or a homolog thereof as depicted in column 8 of Table II, application no. 14, preferably a homolog or functional equivalent as depicted in column 8 of Table II B, application no. 14, and being depicted in the same respective line as said GM02LC19289, and preferably the activity is increased non-targeted, whereby the respective line disclose in column 7 the fine chemical beta-sitosterol.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 46751, preferably the coding region thereof, conferred the production of or the increase in beta-sitosterol compared with the wild type control.

The nucleic acid sequence of GM02LC19289 from Glycine max, e.g. as shown in the respective line in column 5 of Table I, application no. 14, is unpublished. And the activity of the gene product thereof is the activity of transcription factor.

Accordingly, in one embodiment, the process of the present invention for producing campesterol in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said GM02LC19289, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said GM02LC19289, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said GM02LC19289, or a functional equivalent or a homolog thereof as depicted in column 8 of Table II, application no. 14, preferably a homolog or functional equivalent as depicted in column 8 of Table II B, application no. 14, and being depicted in the same respective line as said GM02LC19289, and preferably the activity is increased non-targeted, whereby the respective line disclose in column 7 the fine chemical campesterol.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 46751, preferably the coding region thereof, conferred the production of or the increase in campesterol compared with the wild type control.

The nucleic acid sequence of GM02LC44512 from Glycine max, e.g. as shown in the respective line in column 5 of Table I, application no. 14, is unpublished. And the activity of the gene product thereof is the activity of cyclin D.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “cyclin D”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said GM02LC44512, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said GM02LC44512, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said GM02LC44512, or a functional equivalent or a homolog thereof as depicted in column 8 of Table II, application no. 14, preferably a homolog or functional equivalent as depicted in column 8 of Table II B, application no. 14, and being depicted in the same respective line as said GM02LC44512, and preferably the activity is increased non-targeted, whereby the respective line disclose in column 7 the fine chemical beta-sitosterol.

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 46850, preferably the coding region thereof, conferred the production of or the increase in beta-sitosterol compared with the wild type control.

The nucleic acid sequence of GM02LC44512 from Glycine max, e.g. as shown in the respective line in column 5 of Table I, application no. 14, is unpublished. And the activity of the gene product thereof is the activity of cyclin D.

Accordingly, in one embodiment, the process of the present invention for producing campesterol in a non-human organism, like a microorganism or a plant or a part thereof, comprises increasing or generating the activity of a gene product with the activity of a gene product conferring the activity of “cyclin D”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said GM02LC44512, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said GM02LC44512, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said GM02LC44512, or a functional equivalent or a homolog thereof as depicted in column 8 of Table II, application no. 14, preferably a homolog or functional equivalent as depicted in column 8 of Table II B, application no. 14, and being depicted in the same respective line as said GM02LC44512, and preferably the activity is increased non-targeted, whereby the respective line disclose in column 7 the fine chemical campesterol.

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 46850, preferably the coding region thereof, conferred the production of or the increase in campesterol compared with the wild type control.

The nucleic acid sequence of Sll0406 from Synechocystis sp., e.g. as shown in the respective line in column 5 of Table I, application no. 14, has been published in C. And the activity of the gene product thereof is the activity of sll0406-protein.

Accordingly, in one embodiment, the process of the present invention for producing campesterol in a non-human organism, like a microorganism or a plant or a part thereof, comprises increasing or generating the activity of a gene product with the activity of a gene product conferring the activity of “sll0406-protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said Sll0406, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said Sll0406, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said Sll0406, or a functional equivalent or a homolog thereof as depicted in column 8 of Table II, application no. 14, preferably a homolog or functional equivalent as depicted in column 8 of Table II B, application no. 14, and being depicted in the same respective line as said Sll0406, and preferably the activity is increased non-targeted, whereby the respective line disclose in column 7 the fine chemical campesterol.

Accordingly, in one embodiment, the molecule which activity is to be increased in the process of the invention is the gene product with an activity as a “sll0406-protein”, preferably it is the molecule of section (a) or (b) of this paragraph.

In particular, it was observed that in plants, especially in Arabidopsis thaliana, increasing or generating the activity of a gene product non-targeted with the activity of a “sll0406-protein”, preferably being encoded by a gene comprising the nucleic acid sequence SEQ ID NO. 127103, preferably the coding region thereof, conferred the production of or the increase in campesterol compared with the wild type control.

The nucleic acid sequence of Sll0671 from Synechocystis sp., e.g. as shown in the respective line in column 5 of Table I, application no. 14, has been published in C. And the activity of the gene product thereof is the activity of cation transport protein.

Accordingly, in one embodiment, the process of the present invention for producing campesterol in a non-human organism, like a microorganism or a plant or a part thereof, comprises increasing or generating the activity of a gene product with the activity of a gene product conferring the activity of “cation transport protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said Sll0671, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said Sll0671, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said Sll0671, or a functional equivalent or a homolog thereof as depicted in column 8 of Table II, application no. 14, preferably a homolog or functional equivalent as depicted in column 8 of Table II B, application no. 14, and being depicted in the same respective line as said Sll0671, and preferably the activity is increased non-targeted, whereby the respective line disclose in column 7 the fine chemical campesterol.

Accordingly, in one embodiment, the molecule which activity is to be increased in the process of the invention is the gene product with an activity as a “cation transport protein”, preferably it is the molecule of section (a) or (b) of this paragraph.

In particular, it was observed that in plants, especially in Arabidopsis thaliana, increasing or generating the activity of a gene product non-targeted with the activity of a “cation transport protein”, preferably being encoded by a gene comprising the nucleic acid sequence SEQ ID NO. 132874, preferably the coding region thereof, conferred the production of or the increase in campesterol compared with the wild type control.

The nucleic acid sequence of Sll1443 from Synechocystis sp., e.g. as shown in the respective line in column 5 of Table I, application no. 14, has been published in C. And the activity of the gene product thereof is the activity of CTP synthetase.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “CTP synthetase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said Sll1443, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said Sll1443, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said Sll1443, or a functional equivalent or a homolog thereof as depicted in column 8 of Table II, application no. 14, preferably a homolog or functional equivalent as depicted in column 8 of Table II B, application no. 14, and being depicted in the same respective line as said Sll1443, and preferably the activity is increased mitochondrial, whereby the respective line disclose in column 7 the fine chemical beta-sitosterol.

Accordingly, in one embodiment, the molecule which activity is to be increased in the process of the invention is the gene product with an activity as a “CTP synthetase”, preferably it is the molecule of section (a) or (b) of this paragraph.

In particular, it was observed that in plants, especially in Arabidopsis thaliana, increasing or generating the activity of a gene product mitochondrial with the activity of a “CTP synthetase”, preferably being encoded by a gene comprising the nucleic acid sequence SEQ ID NO. 53878, preferably the coding region thereof, conferred the production of or the increase in beta-sitosterol compared with the wild type control.

The nucleic acid sequence of Sll1443 from Synechocystis sp., e.g. as shown in the respective line in column 5 of Table I, application no. 14, has been published in C. And the activity of the gene product thereof is the activity of CTP synthetase.

Accordingly, in one embodiment, the process of the present invention for producing campesterol in a non-human organism, like a microorganism or a plant or a part thereof, comprises increasing or generating the activity of a gene product with the activity of a gene product conferring the activity of “CTP synthetase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said Sll1443, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said Sll1443, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said Sll1443, or a functional equivalent or a homolog thereof as depicted in column 8 of Table II, application no. 14, preferably a homolog or functional equivalent as depicted in column 8 of Table II B, application no. 14, and being depicted in the same respective line as said Sll1443, and preferably the activity is increased mitochondrial, whereby the respective line disclose in column 7 the fine chemical campesterol.

Accordingly, in one embodiment, the molecule which activity is to be increased in the process of the invention is the gene product with an activity as a “CTP synthetase”, preferably it is the molecule of section (a) or (b) of this paragraph.

In particular, it was observed that in plants, especially in Arabidopsis thaliana, increasing or generating the activity of a gene product mitochondrial with the activity of a “CTP synthetase”, preferably being encoded by a gene comprising the nucleic acid sequence SEQ ID NO. 53878, preferably the coding region thereof, conferred the production of or the increase in campesterol compared with the wild type control.

The nucleic acid sequence of Sll1525 from Synechocystis sp., e.g. as shown in the respective line in column 5 of Table I, application no. 14, has been published in C. And the activity of the gene product thereof is the activity of phosphoribulokinase.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “phosphoribulokinase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said Sll1525, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said Sll1525, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said Sll1525, or a functional equivalent or a homolog thereof as depicted in column 8 of Table II, application no. 14, preferably a homolog or functional equivalent as depicted in column 8 of Table II B, application no. 14, and being depicted in the same respective line as said Sll1525, and preferably the activity is increased mitochondrial, whereby the respective line disclose in column 7 the fine chemical beta-sitosterol.

Accordingly, in one embodiment, the molecule which activity is to be increased in the process of the invention is the gene product with an activity as a “phosphoribulokinase”, preferably it is the molecule of section (a) or (b) of this paragraph.

In particular, it was observed that in plants, especially in Arabidopsis thaliana, increasing or generating the activity of a gene product mitochondrial with the activity of a “phosphoribulokinase”, preferably being encoded by a gene comprising the nucleic acid sequence SEQ ID NO. 132938, preferably the coding region thereof, conferred the production of or the increase in beta-sitosterol compared with the wild type control.

The nucleic acid sequence of Sll1598 from Synechocystis sp., e.g. as shown in the respective line in column 5 of Table I, application no. 14, has been published in C. And the activity of the gene product thereof is the activity of ABC metal ion transporter substrate-binding protein.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “ABC metal ion transporter substrate-binding protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said Sll1598, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said Sll1598, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said Sll1598, or a functional equivalent or a homolog thereof as depicted in column 8 of Table II, application no. 14, preferably a homolog or functional equivalent as depicted in column 8 of Table II B, application no. 14, and being depicted in the same respective line as said Sll1598, and preferably the activity is increased mitochondrial, whereby the respective line disclose in column 7 the fine chemical beta-sitosterol.

Accordingly, in one embodiment, the molecule which activity is to be increased in the process of the invention is the gene product with an activity as a “ABC metal ion transporter substratebinding protein”, preferably it is the molecule of section (a) or (b) of this paragraph.

In particular, it was observed that in plants, especially in Arabidopsis thaliana, increasing or generating the activity of a gene product mitochondrial with the activity of a “ABC metal ion transporter substrate-binding protein”, preferably being encoded by a gene comprising the nucleic acid sequence SEQ ID NO. 85743, preferably the coding region thereof, conferred the production of or the increase in beta-sitosterol compared with the wild type control.

The nucleic acid sequence of Sll1761 from Synechocystis sp., e.g. as shown in the respective line in column 5 of Table I, application no. 14, has been published in C. And the activity of the gene product thereof is the activity of sll1761-protein.

Accordingly, in one embodiment, the process of the present invention for producing campesterol in a non-human organism, like a microorganism or a plant or a part thereof, comprises increasing or generating the activity of a gene product with the activity of a gene product conferring the activity of “sll1761-protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said Sll1761, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said Sll1761, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said Sll1761, or a functional equivalent or a homolog thereof as depicted in column 8 of Table II, application no. 14, preferably a homolog or functional equivalent as depicted in column 8 of Table II B, application no. 14, and being depicted in the same respective line as said Sll1761, and preferably the activity is increased non-targeted, whereby the respective line disclose in column 7 the fine chemical campesterol.

Accordingly, in one embodiment, the molecule which activity is to be increased in the process of the invention is the gene product with an activity as a “sll1761-protein”, preferably it is the molecule of section (a) or (b) of this paragraph.

In particular, it was observed that in plants, especially in Arabidopsis thaliana, increasing or generating the activity of a gene product non-targeted with the activity of a “sll1761-protein”, preferably being encoded by a gene comprising the nucleic acid sequence SEQ ID NO. 55379, preferably the coding region thereof, conferred the production of or the increase in campesterol compared with the wild type control.

The nucleic acid sequence of Sll1848 from Synechocystis sp., e.g. as shown in the respective line in column 5 of Table I, application no. 14, has been published in C. And the activity of the gene product thereof is the activity of acyl transferase.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “acyl transferase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said Sll1848, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said Sll1848, and preferably the activity is increased plastidic, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said Sll1848, or a functional equivalent or a homolog thereof as depicted in column 8 of Table II, application no. 14, preferably a homolog or functional equivalent as depicted in column 8 of Table II B, application no. 14, and being depicted in the same respective line as said Sll1848, and preferably the activity is increased plastidic, whereby the respective line disclose in column 7 the fine chemical beta-sitosterol.

Accordingly, in one embodiment, the molecule which activity is to be increased in the process of the invention is the gene product with an activity as a “acyl transferase”, preferably it is the molecule of section (a) or (b) of this paragraph.

In particular, it was observed that in plants, especially in Arabidopsis thaliana, increasing or generating the activity of a gene product plastidic with the activity of a “acyl transferase”, preferably being encoded by a gene comprising the nucleic acid sequence SEQ ID NO. 86447, preferably the coding region thereof, conferred the production of or the increase in beta-sitosterol compared with the wild type control.

The nucleic acid sequence of Sll1920 from Synechocystis sp., e.g. as shown in the respective line in column 5 of Table I, application no. 14, has been published in C. And the activity of the gene product thereof is the activity of cation-transporting ATPase.

Accordingly, in one embodiment, the process of the present invention for producing campesterol in a non-human organism, like a microorganism or a plant or a part thereof, comprises increasing or generating the activity of a gene product with the activity of a gene product conferring the activity of “cation-transporting ATPase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said Sll1920, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said Sll1920, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said Sll1920, or a functional equivalent or a homolog thereof as depicted in column 8 of Table II, application no. 14, preferably a homolog or functional equivalent as depicted in column 8 of Table II B, application no. 14, and being depicted in the same respective line as said Sll1920, and preferably the activity is increased mitochondrial, whereby the respective line disclose in column 7 the fine chemical campesterol.

Accordingly, in one embodiment, the molecule which activity is to be increased in the process of the invention is the gene product with an activity as a “cation-transporting ATPase”, preferably it is the molecule of section (a) or (b) of this paragraph.

In particular, it was observed that in plants, especially in Arabidopsis thaliana, increasing or generating the activity of a gene product mitochondrial with the activity of a “cation-transporting ATPase”, preferably being encoded by a gene comprising the nucleic acid sequence SEQ ID NO. 55385, preferably the coding region thereof, conferred the production of or the increase in campesterol compared with the wild type control.

The nucleic acid sequence of Slr0338 from Synechocystis sp., e.g. as shown in the respective line in column 5 of Table I, application no. 14, has been published in C. And the activity of the gene product thereof is the activity of oxidoreductase.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “oxidoreductase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said Slr0338, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said Slr0338, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said Slr0338, or a functional equivalent or a homolog thereof as depicted in column 8 of Table II, application no. 14, preferably a homolog or functional equivalent as depicted in column 8 of Table II B, application no. 14, and being depicted in the same respective line as said Slr0338, and preferably the activity is increased non-targeted, whereby the respective line disclose in column 7 the fine chemical beta-sitosterol.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 11990, preferably the coding region thereof, conferred the production of or the increase in beta-sitosterol compared with the wild type control.

The nucleic acid sequence of Slr0949 from Synechocystis sp., e.g. as shown in the respective line in column 5 of Table I, application no. 14, has been published in C. And the activity of the gene product thereof is the activity of permease protein of ABC transporter.

Accordingly, in one embodiment, the process of the present invention for producing campesterol in a non-human organism, like a microorganism or a plant or a part thereof, comprises increasing or generating the activity of a gene product with the activity of a gene product conferring the activity of “permease protein of ABC transporter”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said Slr0949, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said Slr0949, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said Slr0949, or a functional equivalent or a homolog thereof as depicted in column 8 of Table II, application no. 14, preferably a homolog or functional equivalent as depicted in column 8 of Table II B, application no. 14, and being depicted in the same respective line as said Slr0949, and preferably the activity is increased mitochondrial, whereby the respective line disclose in column 7 the fine chemical campesterol.

Accordingly, in one embodiment, the molecule which activity is to be increased in the process of the invention is the gene product with an activity as a “permease protein of ABC transporter”, preferably it is the molecule of section (a) or (b) of this paragraph.

In particular, it was observed that in plants, especially in Arabidopsis thaliana, increasing or generating the activity of a gene product mitochondrial with the activity of a “permease protein of ABC transporter”, preferably being encoded by a gene comprising the nucleic acid sequence SEQ ID NO. 119222, preferably the coding region thereof, conferred the production of or the increase in campesterol compared with the wild type control.

The nucleic acid sequence of Slr0966 from Synechocystis sp., e.g. as shown in the respective line in column 5 of Table I, application no. 14, has been published in C. And the activity of the gene product thereof is the activity of tryptophan synthase alpha chain.

Accordingly, in one embodiment, the process of the present invention for producing campesterol in a non-human organism, like a microorganism or a plant or a part thereof, comprises increasing or generating the activity of a gene product with the activity of a gene product conferring the activity of “tryptophan synthase alpha chain”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said Slr0966, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said Slr0966, and preferably the activity is increased plastidic, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said Slr0966, or a functional equivalent or a homolog thereof as depicted in column 8 of Table II, application no. 14, preferably a homolog or functional equivalent as depicted in column 8 of Table II B, application no. 14, and being depicted in the same respective line as said Slr0966, and preferably the activity is increased plastidic, whereby the respective line disclose in column 7 the fine chemical campesterol.

Accordingly, in one embodiment, the molecule which activity is to be increased in the process of the invention is the gene product with an activity as a “tryptophan synthase alpha chain”, preferably it is the molecule of section (a) or (b) of this paragraph.

In particular, it was observed that in plants, especially in Arabidopsis thaliana, increasing or generating the activity of a gene product plastidic with the activity of a “tryptophan synthase alpha chain”, preferably being encoded by a gene comprising the nucleic acid sequence SEQ ID NO. 87655, preferably the coding region thereof, conferred the production of or the increase in campesterol compared with the wild type control.

The nucleic acid sequence of TTC1193 from Thermus thermophilus, e.g. as shown in the respective line in column 5 of Table I, application no. 14, has been published in E. And the activity of the gene product thereof is the activity of multiple antibiotic resistance protein.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “multiple antibiotic resistance protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said TTC1193, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said TTC1193, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said TTC1193, or a functional equivalent or a homolog thereof as depicted in column 8 of Table II, application no. 14, preferably a homolog or functional equivalent as depicted in column 8 of Table II B, application no. 14, and being depicted in the same respective line as said TTC1193, and preferably the activity is increased non-targeted, whereby the respective line disclose in column 7 the fine chemical beta-sitosterol.

Accordingly, in one embodiment, the molecule which activity is to be increased in the process of the invention is the gene product with an activity as a “multiple antibiotic resistance protein”, preferably it is the molecule of section (a) or (b) of this paragraph.

In particular, it was observed that in plants, especially in Arabidopsis thaliana, increasing or generating the activity of a gene product non-targeted with the activity of a “multiple antibiotic resistance protein”, preferably being encoded by a gene comprising the nucleic acid sequence SEQ ID NO. 61723, preferably the coding region thereof, conferred the production of or the increase in beta-sitosterol compared with the wild type control.

The nucleic acid sequence of TTC1193 from Thermus thermophilus, e.g. as shown in the respective line in column 5 of Table I, application no. 14, has been published in E. And the activity of the gene product thereof is the activity of multiple antibiotic resistance protein.

Accordingly, in one embodiment, the process of the present invention for producing campesterol in a non-human organism, like a microorganism or a plant or a part thereof, comprises increasing or generating the activity of a gene product with the activity of a gene product conferring the activity of “multiple antibiotic resistance protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said TTC1193, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said TTC1193, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said TTC1193, or a functional equivalent or a homolog thereof as depicted in column 8 of Table II, application no. 14, preferably a homolog or functional equivalent as depicted in column 8 of Table II B, application no. 14, and being depicted in the same respective line as said TTC1193, and preferably the activity is increased non-targeted, whereby the respective line disclose in column 7 the fine chemical campesterol.

Accordingly, in one embodiment, the molecule which activity is to be increased in the process of the invention is the gene product with an activity as a “multiple antibiotic resistance protein”, preferably it is the molecule of section (a) or (b) of this paragraph.

In particular, it was observed that in plants, especially in Arabidopsis thaliana, increasing or generating the activity of a gene product non-targeted with the activity of a “multiple antibiotic resistance protein”, preferably being encoded by a gene comprising the nucleic acid sequence SEQ ID NO. 61723, preferably the coding region thereof, conferred the production of or the increase in campesterol compared with the wild type control.

The nucleic acid sequence of TTC1386 from Thermus thermophilus, e.g. as shown in the respective line in column 5 of Table I, application no. 14, has been published in E. And the activity of the gene product thereof is the activity of TTC1386-protein.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “TTC1386-protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said TTC1386, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said TTC1386, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said TTC1386, or a functional equivalent or a homolog thereof as depicted in column 8 of Table II, application no. 14, preferably a homolog or functional equivalent as depicted in column 8 of Table II B, application no. 14, and being depicted in the same respective line as said TTC1386, and preferably the activity is increased non-targeted, whereby the respective line disclose in column 7 the fine chemical beta-sitosterol.

Accordingly, in one embodiment, the molecule which activity is to be increased in the process of the invention is the gene product with an activity as a “TTC1386-protein”, preferably it is the molecule of section (a) or (b) of this paragraph.

In particular, it was observed that in plants, especially in Arabidopsis thaliana, increasing or generating the activity of a gene product non-targeted with the activity of a “TTC1386-protein”, preferably being encoded by a gene comprising the nucleic acid sequence SEQ ID NO. 62079, preferably the coding region thereof, conferred the production of or the increase in beta-sitosterol compared with the wild type control.

The nucleic acid sequence of TTC1386 from Thermus thermophilus, e.g. as shown in the respective line in column 5 of Table I, application no. 14, has been published in E. And the activity of the gene product thereof is the activity of TTC1386-protein.

Accordingly, in one embodiment, the process of the present invention for producing campesterol in a non-human organism, like a microorganism or a plant or a part thereof, comprises increasing or generating the activity of a gene product with the activity of a gene product conferring the activity of “TTC1386-protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said TTC1386, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said TTC1386, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said TTC1386, or a functional equivalent or a homolog thereof as depicted in column 8 of Table II, application no. 14, preferably a homolog or functional equivalent as depicted in column 8 of Table II B, application no. 14, and being depicted in the same respective line as said TTC1386, and preferably the activity is increased non-targeted, whereby the respective line disclose in column 7 the fine chemical campesterol.

Accordingly, in one embodiment, the molecule which activity is to be increased in the process of the invention is the gene product with an activity as a “TTC1386-protein”, preferably it is the molecule of section (a) or (b) of this paragraph.

In particular, it was observed that in plants, especially in Arabidopsis thaliana, increasing or generating the activity of a gene product non-targeted with the activity of a “TTC1386-protein”, preferably being encoded by a gene comprising the nucleic acid sequence SEQ ID NO. 62079, preferably the coding region thereof, conferred the production of or the increase in campesterol compared with the wild type control.

The nucleic acid sequence of TTC1550 from Thermus thermophilus, e.g. as shown in the respective line in column 5 of Table I, application no. 14, has been published in E. And the activity of the gene product thereof is the activity of homocitrate synthase.

Accordingly, in one embodiment, the process of the present invention for producing campesterol in a non-human organism, like a microorganism or a plant or a part thereof, comprises increasing or generating the activity of a gene product with the activity of a gene product conferring the activity of “homocitrate synthase”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said TTC1550, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said TTC1550, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said TTC1550, or a functional equivalent or a homolog thereof as depicted in column 8 of Table II, application no. 14, preferably a homolog or functional equivalent as depicted in column 8 of Table II B, application no. 14, and being depicted in the same respective line as said TTC1550, and preferably the activity is increased non-targeted, whereby the respective line disclose in column 7 the fine chemical campesterol.

Accordingly, in one embodiment, the molecule which activity is to be increased in the process of the invention is the gene product with an activity as a “homocitrate synthase”, preferably it is the molecule of section (a) or (b) of this paragraph.

In particular, it was observed that in plants, especially in Arabidopsis thaliana, increasing or generating the activity of a gene product non-targeted with the activity of a “homocitrate synthase”, preferably being encoded by a gene comprising the nucleic acid sequence SEQ ID NO. 12974, preferably the coding region thereof, conferred the production of or the increase in campesterol compared with the wild type control.

The nucleic acid sequence of Ydl131w from Saccharomyces cerevisiae, e.g. as shown in the respective line in column 5 of Table I, application no. 14, has been published in A. And the activity of the gene product thereof is the activity of homocitrate synthase.

Accordingly, in one embodiment, the process of the present invention for producing campesterol in a non-human organism, like a microorganism or a plant or a part thereof, comprises increasing or generating the activity of a gene product with the activity of a gene product conferring the activity of “homocitrate synthase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said Ydl131w, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said Ydl131w, and preferably the activity is increased plastidic, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said Ydl131w, or a functional equivalent or a homolog thereof as depicted in column 8 of Table II, application no. 14, preferably a homolog or functional equivalent as depicted in column 8 of Table II B, application no. 14, and being depicted in the same respective line as said Ydl131w, and preferably the activity is increased plastidic, whereby the respective line disclose in column 7 the fine chemical campesterol.

Accordingly, in one embodiment, the molecule which activity is to be increased in the process of the invention is the gene product with an activity as a “homocitrate synthase”, preferably it is the molecule of section (a) or (b) of this paragraph.

In particular, it was observed that in plants, especially in Arabidopsis thaliana, increasing or generating the activity of a gene product plastidic with the activity of a “homocitrate synthase”, preferably being encoded by a gene comprising the nucleic acid sequence SEQ ID NO. 113224, preferably the coding region thereof, conferred the production of or the increase in campesterol compared with the wild type control.

The nucleic acid sequence of Ydl155w from Saccharomyces cerevisiae, e.g. as shown in the respective line in column 5 of Table I, application no. 14, has been published in A. And the activity of the gene product thereof is the activity of cyclin.

Accordingly, in one embodiment, the process of the present invention for producing campesterol in a non-human organism, like a microorganism or a plant or a part thereof, comprises increasing or generating the activity of a gene product with the activity of a gene product conferring the activity of “cyclin”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said Ydl155w, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said Ydl155w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said Ydl155w, or a functional equivalent or a homolog thereof as depicted in column 8 of Table II, application no. 14, preferably a homolog or functional equivalent as depicted in column 8 of Table II B, application no. 14, and being depicted in the same respective line as said Ydl155w, and preferably the activity is increased non-targeted, whereby the respective line disclose in column 7 the fine chemical campesterol.

Accordingly, in one embodiment, the molecule which activity is to be increased in the process of the invention is the gene product with an activity as a “cyclin”, preferably it is the molecule of section (a) or (b) of this paragraph.

In particular, it was observed that in plants, especially in Arabidopsis thaliana, increasing or generating the activity of a gene product non-targeted with the activity of a “cyclin”, preferably being encoded by a gene comprising the nucleic acid sequence SEQ ID NO. 14171, preferably the coding region thereof, conferred the production of or the increase in campesterol compared with the wild type control.

The nucleic acid sequence of Ydl235c from Saccharomyces cerevisiae, e.g. as shown in the respective line in column 5 of Table I, application no. 14, has been published in A. And the activity of the gene product thereof is the activity of ydl235c-protein.

Accordingly, in one embodiment, the process of the present invention for producing campesterol in a non-human organism, like a microorganism or a plant or a part thereof, comprises increasing or generating the activity of a gene product with the activity of a gene product conferring the activity of “ydl235c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said Ydl235c, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said Ydl235c, and preferably the activity is increased plastidic, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said Ydl235c, or a functional equivalent or a homolog thereof as depicted in column 8 of Table II, application no. 14, preferably a homolog or functional equivalent as depicted in column 8 of Table II B, application no. 14, and being depicted in the same respective line as said Ydl235c, and preferably the activity is increased plastidic, whereby the respective line disclose in column 7 the fine chemical campesterol.

Accordingly, in one embodiment, the molecule which activity is to be increased in the process of the invention is the gene product with an activity as a “ydl235c-protein”, preferably it is the molecule of section (a) or (b) of this paragraph.

In particular, it was observed that in plants, especially in Arabidopsis thaliana, increasing or generating the activity of a gene product plastidic with the activity of a “ydl235c-protein”, preferably being encoded by a gene comprising the nucleic acid sequence SEQ ID NO. 113313, preferably the coding region thereof, conferred the production of or the increase in campesterol compared with the wild type control.

The nucleic acid sequence of Ygl039w from Saccharomyces cerevisiae, e.g. as shown in the respective line in column 5 of Table I, application no. 14, has been published in A. And the activity of the gene product thereof is the activity of oxidoreductase.

Accordingly, in one embodiment, the process of the present invention for producing campesterol in a non-human organism, like a microorganism or a plant or a part thereof, comprises increasing or generating the activity of a gene product with the activity of a gene product conferring the activity of “oxidoreductase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said Ygl039w, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said Ygl039w, and preferably the activity is increased plastidic, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said Ygl039w, or a functional equivalent or a homolog thereof as depicted in column 8 of Table II, application no. 14, preferably a homolog or functional equivalent as depicted in column 8 of Table II B, application no. 14, and being depicted in the same respective line as said Ygl039w, and preferably the activity is increased plastidic, whereby the respective line disclose in column 7 the fine chemical campesterol.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product plastidic with the activity of a “oxidoreductase”, preferably being encoded by a gene comprising the nucleic acid sequence SEQ ID NO. 120436, preferably the coding region thereof, conferred the production of or the increase in campesterol compared with the wild type control.

The nucleic acid sequence of Yjr153w from Saccharomyces cerevisiae, e.g. as shown in the respective line in column 5 of Table I, application no. 14, has been published in A. And the activity of the gene product thereof is the activity of polygalacturonase.

Accordingly, in one embodiment, the process of the present invention for producing campesterol in a non-human organism, like a microorganism or a plant or a part thereof, comprises increasing or generating the activity of a gene product with the activity of a gene product conferring the activity of “polygalacturonase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said Yjr153w, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said Yjr153w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said Yjr153w, or a functional equivalent or a homolog thereof as depicted in column 8 of Table II, application no. 14, preferably a homolog or functional equivalent as depicted in column 8 of Table II B, application no. 14, and being depicted in the same respective line as said Yjr153w, and preferably the activity is increased non-targeted, whereby the respective line disclose in column 7 the fine chemical campesterol.

Accordingly, in one embodiment, the molecule which activity is to be increased in the process of the invention is the gene product with an activity as a “polygalacturonase”, preferably it is the molecule of section (a) or (b) of this paragraph.

In particular, it was observed that in plants, especially in Arabidopsis thaliana, increasing or generating the activity of a gene product non-targeted with the activity of a “polygalacturonase”, preferably being encoded by a gene comprising the nucleic acid sequence SEQ ID NO. 66274, preferably the coding region thereof, conferred the production of or the increase in campesterol compared with the wild type control.

The nucleic acid sequence of Ynl321w from Saccharomyces cerevisiae, e.g. as shown in the respective line in column 5 of Table I, application no. 14, has been published in A. And the activity of the gene product thereof is the activity of ynl321w-protein.

Accordingly, in one embodiment, the process of the present invention for producing campesterol in a non-human organism, like a microorganism or a plant or a part thereof, comprises increasing or generating the activity of a gene product with the activity of a gene product conferring the activity of “ynl321w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said Ynl321w, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said Ynl321w, and preferably the activity is increased plastidic, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said Ynl321w, or a functional equivalent or a homolog thereof as depicted in column 8 of Table II, application no. 14, preferably a homolog or functional equivalent as depicted in column 8 of Table II B, application no. 14, and being depicted in the same respective line as said Ynl321w, and preferably the activity is increased plastidic, whereby the respective line disclose in column 7 the fine chemical campesterol.

Accordingly, in one embodiment, the molecule which activity is to be increased in the process of the invention is the gene product with an activity as a “ynl321w-protein”, preferably it is the molecule of section (a) or (b) of this paragraph.

In particular, it was observed that in plants, especially in Arabidopsis thaliana, increasing or generating the activity of a gene product plastidic with the activity of a “ynl321w-protein”, preferably being encoded by a gene comprising the nucleic acid sequence SEQ ID NO. 129546, preferably the coding region thereof, conferred the production of or the increase in campesterol compared with the wild type control.

The nucleic acid sequence of Ypl028w from Saccharomyces cerevisiae, e.g. as shown in the respective line in column 5 of Table I, application no. 14, has been published in A. And the activity of the gene product thereof is the activity of acetyl-CoA acetyltransferase.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “acetyl-CoA acetyltransferase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said Ypl028w, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said Ypl028w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said Ypl028w, or a functional equivalent or a homolog thereof as depicted in column 8 of Table II, application no. 14, preferably a homolog or functional equivalent as depicted in column 8 of Table II B, application no. 14, and being depicted in the same respective line as said Ypl028w, and preferably the activity is increased non-targeted, whereby the respective line disclose in column 7 the fine chemical beta-sitosterol.

Accordingly, in one embodiment, the molecule which activity is to be increased in the process of the invention is the gene product with an activity as a “acetyl-CoA acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyl-CoA acetyltransferase”, preferably being encoded by a gene comprising the nucleic acid sequence SEQ ID NO. 92668, preferably the coding region thereof, conferred the production of or the increase in beta-sitosterol compared with the wild type control.

The nucleic acid sequence of Ypr170c from Saccharomyces cerevisiae, e.g. as shown in the respective line in column 5 of Table I, application no. 14, has been published in A. And the activity of the gene product thereof is the activity of ypr170c-protein.

Accordingly, in one embodiment, the process of the present invention for producing beta-sitosterol in a non-human organism, like a microorganism or a plant or a part thereof, comprises increasing or generating the activity of a gene product with the activity of a gene product conferring the activity of “ypr170c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical beta-sitosterol), application no. 14, preferably the coding region thereof, or a homolog or a fragment thereof, and being depicted in the same respective line as said Ypr170c, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said Ypr170c, and preferably the activity is increased plastidic, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said Ypr170c, or a functional equivalent or a homolog thereof as depicted in column 8 of Table II, application no. 14, preferably a homolog or functional equivalent as depicted in column 8 of Table II B, application no. 14, and being depicted in the same respective line as said Ypr170c, and preferably the activity is increased plastidic, whereby the respective line disclose in column 7 the fine chemical beta-sitosterol.

Accordingly, in one embodiment, the molecule which activity is to be increased in the process of the invention is the gene product with an activity as a “ypr170c-protein”, preferably it is the molecule of section (a) or (b) of this paragraph.

In particular, it was observed that in plants, especially in Arabidopsis thaliana, increasing or generating the activity of a gene product plastidic with the activity of a “ypr170c-protein”, preferably being encoded by a gene comprising the nucleic acid sequence SEQ ID NO. 113647, preferably the coding region thereof, conferred the production of or the increase in beta-sitosterol compared with the wild type control.

The nucleic acid sequence of 59582753_SOYBEAN from Glycine max, e.g. as shown in the respective line in column 5 of Table I, application no. 14, is unpublished. And the activity of the gene product thereof is the activity of glucose-6-phosphate 1-dehydrogenase.

Accordingly, in one embodiment, the process of the present invention for producing campesterol in a non-human organism, like a microorganism or a plant or a part thereof, comprises increasing or generating the activity of a gene product with the activity of a gene product conferring the activity of “glucose-6-phosphate 1-dehydrogenase”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical campesterol), application no. 14, preferably the coding region thereof, or a homolog or a fragment thereof, and being depicted in the same respective line as said 59582753_SOYBEAN, or a functional equivalent or a homolog thereof as shown in column 8 of Table I, application no. 14, preferably the coding region thereof, particularly a homolog or functional equivalent as shown in column 8 of Table I B, application no. 14, and being depicted in the same respective line as said 59582753_SOYBEAN, and preferably the activity is increased plastidic, or
(b) a polypeptide comprising a polypeptide, a consensus sequence or at least a polypeptide motif as shown in the respective line in column 5 of Table II or in column 8 of Table IV, application no. 14, respectively, and being depicted in the same respective line as said 59582753_SOYBEAN, or a functional equivalent or a homolog thereof as depicted in column 8 of Table II, application no. 14, preferably a homolog or functional equivalent as depicted in column 8 of Table II B, application no. 14, and being depicted in the same respective line as said 59582753_SOYBEAN, and preferably the activity is increased plastidic, whereby the respective line disclose in column 7 the fine chemical campesterol.

Accordingly, in one embodiment, the molecule which activity is to be increased in the process of the invention is the gene product with an activity as a “glucose-6-phosphate 1-dehydrogenase”, preferably it is the molecule of section (a) or (b) of this paragraph.

In particular, it was observed that in plants, especially in Arabidopsis thaliana, increasing or generating the activity of a gene product plastidic with the activity of a “glucose-6-phosphate 1dehydrogenase”, preferably being encoded by a gene comprising the nucleic acid sequence SEQ ID NO. 16883, preferably the coding region thereof, conferred the production of or the increase in campesterol compared with the wild type control.

[0105.1.7.14] to [0107.1.7.14] for the disclosure of these paragraphs see [0105.1.7.7] to [0107.1.7.7] above

A protein having an activity conferring an increase in the amount or level of the fine chemical beta-sitosterol, campesterol, or stigmasterol, upon targeting to the plastids or mitochondria or upon non-targeting, preferably has the structure of the respective polypeptide described herein, in particular of the polypeptides comprising the consensus sequence or at least one polypeptide motifs as shown in the respective line in Table IV, application no. 14, column 8 or of the polypeptide comprising an amino acid sequence as disclosed in the respective line in Table II, application no. 14, columns 5 or 8, or homologs or fragments thereof as described herein, or is encoded by the nucleic acid molecule characterized herein or the nucleic acid molecule according to the invention, for example by the nucleic acid molecule as shown in the respective line in Table I, application no. 14, columns 5 or 8, preferably the coding region thereof, or homologs or fragments thereof and has the herein mentioned activity.

for the disclosure of this paragraph see [0109.1.7.7] above

for the disclosure of this paragraph see [0110.1.7.7] above

In one embodiment, the process of the present invention comprises one or more of the following steps
(a) stabilizing a protein conferring the generation or increased expression of a protein encoded by the nucleic acid molecule of the invention or of the polypeptide of the invention, e.g. of a polypeptide having the activity selected from the group consisting of 2-oxoglutarate dehydrogenase E1 subunit, ABC metal ion transporter substrate-binding protein, acetyl-CoA acetyltransferase, acetyltransferase, acid shock protein, acyl transferase, amidophosphoribosyltransferase, ankyrin repeat family protein, ATP-binding component of a transport system, b1401-protein, B1795-protein, b2107-protein, b2376-protein, b2399-protein, b2474-protein, b2513-protein, b2654-protein, b3151-protein, b3989-protein, b4029-protein, b4205-protein, C-4 sterol methyl oxidase, calcium-dependent protein kinase, cation transport protein, cation-transporting ATPase, CTP synthase, CTP synthetase, cyclin, cyclin D, eukaryotic translation initiation factor 5, glucose dehydrogenase, glucose-6-phosphate 1-dehydrogenase, glutaredoxin, homocitrate synthase, hydrolase, lipoprotein precursor, L-ribulose-5-phosphate 4-epimerase, membrane transport protein, methyltransferase, monothiol glutaredoxin, multidrug resistance protein, multiple antibiotic resistance protein, murein transglycosylase, NADH dehydrogenase I chain N, oxidoreductase, permease protein, permease protein of ABC transporter, phenylacetic acid degradation protein, phosphoribulokinase, polygalacturonase, protein kinase, protein phosphatase, putative transport system permease protein, putrescine transport system permease protein, recombinase A, sec-independent protein translocase, sll0406-protein, sll1761-protein, sodium/proton antiporter, sterol desaturase, threonine dehydrogenase, threonine synthase, transcription factor, tryptophan synthase alpha chain, TTC1386-protein, xanthine dehydrogenase, ydl235c-protein, ynl321w-protein, and ypr170c-protein, or of a polypeptide as indicated in the respective line in Table II, application no. 14, columns 5 or 8, or its homologs or fragments, and conferring the production of or an increase in beta-sitosterol, campesterol, or stigmasterol, respectively; and/or
(b) stabilizing a mRNA conferring the generation or increased expression of a FCRP, e.g. protein encoded by the nucleic acid molecule of the invention or its homologs or fragments, or of a mRNA encoding the polypeptide of the present invention having the hereinmentioned activity selected from the group consisting of said activities mentioned in (a) and conferring the production of or an increase in beta-sitosterol, campesterol, or stigmasterol, respectively; and/or
(c) increasing the specific activity of a protein conferring the increased expression of a FCRP, e.g. a protein encoded by the nucleic acid molecule of the invention or of the polypeptide of the present invention having herein-mentioned beta-sitosterol, campesterol, or stigmasterol generating or increasing activity, respectively, e.g. of a polypeptide having the activity of a protein as indicated in the respective line in Table II, application no. 14, columns 5 or 8, or its homologs or fragments, or decreasing the inhibitory regulation of the polypeptide of the invention; and/or
(d) generating or increasing the expression of an endogenous or artificial transcription factor mediating the expression of a protein conferring the generation or increased expression of a FCRP, e.g. a protein encoded by the nucleic acid molecule of the invention or of the polypeptide of the invention having herein-mentioned activity selected from the group consisting of said activities mentioned in (a) and conferring a beta-sitosterol, campesterol, or stigmasterol increasing activity, respectively, e.g. of a polypeptide having the activity of a protein as indicated in the respective line in Table II, application no. 14, columns 5 or 8 or its homologs; and/or
(e) stimulating activity of a protein conferring the increased expression of a FCRP, e.g. a protein encoded by the nucleic acid molecule of the present invention or a polypeptide of the present invention having herein-mentioned activity selected from the group consisting of said activities mentioned in (a) and conferring a beta-sitosterol, campesterol, or stigmasterol increasing activity, respectively, e.g. of a polypeptide having the activity of a protein as indicated in the respective line in Table II, application no. 14, columns 5 or 8, or its homologs or fragments, by adding one or more exogenous inducing factors to the non-human organism or parts thereof; and/or
(f) expressing a transgenic gene encoding a protein conferring the increased expression of a FCRP, e.g. a polypeptide encoded by the nucleic acid molecule of the present invention or a polypeptide of the present invention, having herein-mentioned activity selected from the group consisting of said activities mentioned in (a) and conferring a beta-sitosterol, campesterol, or stigmasterol increasing activity, respectively, e.g. of a polypeptide having the activity of a protein as indicated in the respective line in Table II, application no. 14, columns 5 or 8, or its homologs or fragments, and/or
(g) increasing the copy number of a gene conferring the increased expression of a nucleic acid molecule encoding a FCRP, e.g. a polypeptide encoded by the nucleic acid molecule of the invention or the polypeptide of the invention having herein-mentioned activity selected from the group consisting of said activities mentioned in (a) and conferring a beta-sitosterol, campesterol, or stigmasterol increasing activity, respectively, e.g. of a polypeptide having the activity of a protein as indicated in the respective line in Table II, application no. 14, columns 5 or 8, or its homologs or fragments; and/or
(h) increasing the expression of the endogenous gene encoding the FCRP, polypeptide of the invention, e.g. a polypeptide encoded by the nucleic acid molecule of the invention or the polypeptide of the invention having herein-mentioned activity selected from the group consisting of said activities mentioned in (a) and conferring a beta-sitosterol, campesterol, or stigmasterol; increasing activity, respectively, e.g. of a polypeptide having the activity of a protein as indicated in the respective line in Table II, application no. 14, columns 5 or 8, or its homologs or fragments, by adding positive expression or removing negative expression elements, e.g. homologous recombination can be used to either introduce positive regulatory elements like for plants the 35S enhancer into the promoter or to remove repressor elements form regulatory regions. Further gene conversion methods can be used to disrupt repressor elements or to enhance to activity of positive elements. Positive elements can be randomly introduced in plants by T-DNA or transposon mutagenesis and lines can be identified in which the positive elements have be integrated near to a gene of the invention, the expression of which is thereby enhanced; and/or
(i) modulating growth conditions of the non-human organism in such a manner, that the expression or activity of the gene encoding the FCRP, e.g. a protein of the invention or the protein itself is enhanced, for example microorganisms or plants can be grown for example under a higher temperature regime leading to an enhanced expression of heat shock proteins, which can lead to an enhanced beta-sitosterol, campesterol, or stigmasterol production, respectively; and/or
(j) selecting of non-human organisms with especially high activity of the proteins of the invention from natural or from mutagenized resources and breeding them into the target organisms, e.g. the elite crops; and/or
(k) directing a protein encoded by the nucleic acid molecule of the invention or of the polypeptide of the present invention having herein-mentioned activity selected from the group consisting of said activities mentioned in (a) and conferring a beta-sitosterol, campesterol, or stigmasterol increasing activity, respectively, e.g. of a polypeptide having the activity of a protein as indicated in the respective line in Table II, application no. 14, columns 5 or 8, or its homologs or fragments, if for the corresponding nucleic acid molecule in the respective line in column 6 of Table I the term “plastidic” is indicated, to the plastids by the addition of a plastidial targeting sequence or if for the corresponding nucleic acid molecule in the respective line in column 6 of Table I the term “mitochondric” is indicated, to the mitochondria by the addition of a mitochondrial targeting sequence; and/or
(l) generating the expression of a protein encoded by the nucleic acid molecule of the invention or of the polypeptide of the present invention having herein-mentioned activity selected from the group consisting of said activities mentioned in (a) and conferring a beta-sitosterol, campesterol, or stigmasterol increasing activity, respectively, e.g. of a polypeptide having the activity of a protein as indicated in the respective line in Table II, application no. 14, columns 5 or 8, or its homologs or fragments, if for the corresponding nucleic acid molecule in the respective line in column 6 of Table I the term “plastidic” or “mitochondric” is indicated, in these organelles by the stable or transient transformation, advantageously stable transformation, of organelles, preferably plastids or mitochondria, with an inventive nucleic acid sequence preferably in form of an expression cassette containing said sequence leading to the expression of the nucleic acids or polypeptides of the invention in the respective organelle; and/or
(m) generating the expression of a protein encoded by the nucleic acid molecule of the invention or of the polypeptide of the present invention having herein-mentioned activity selected from the group consisting of said activities mentioned in (a) and conferring a beta-sitosterol, campesterol, and/or stigmasterol increasing activity, respectively, e.g. of a polypeptide having the activity of a protein as indicated in the respective line in Table II, application no. 14, columns 5 or 8, or its homologs or fragments, if for the corresponding nucleic acid molecule in the respective line in column 6 of Table I the term “plastidic” or “mitochondric” is indicated, in these organelles by integration of a nucleic acid of the invention into the genome of the respective organelle under control of preferable a promoter selective for the respective organelle.

Preferably, said mRNA is the coding region of the nucleic acid molecule of the present invention and/or the protein conferring the increased expression of a protein encoded by the coding region of the nucleic acid molecule of the present invention alone or linked to a transit nucleic acid sequence or transit peptide encoding nucleic acid sequence or the polypeptide having the herein mentioned activity, e.g. conferring the generation of or increase of beta-sitosterol, campesterol, or stigmasterol, respectively, after increasing the expression or activity of the encoded polypeptide, non-targeted or in organelles such as plastids and/or mitochondria, preferably plastids, or having the activity of a polypeptide having an activity as the protein as shown in the respective line in Table II, application no. 14, column 3, or its homologs. Preferably the increase of beta-sitosterol, campesterol, or stigmasterol, respectively, takes place non-targeted or in plastids and/or mitochondria, preferably non-targeted or in plastids.

[0113.1.7.14] to [0122.1.7.14] for the disclosure of these paragraphs see [0113.1.7.7] to [0122.1.7.7] above

The activation of an endogenous polypeptide having above-mentioned activity, e.g. having the activity of a protein as shown in the respective line in Table II, application no. 14, column 3 or of the polypeptide of the invention, e.g. conferring the increase of the fine chemical beta-sitosterol, campesterol, or stigmasterol, respectively, by increase of expression or activity in the cytoplasm, and/or in the cytosol, and/or in an organelle, such as plastids or mitochondria, can also be increased by introducing a synthetic transcription factor, which binds close to the coding region of the gene encoding the protein as shown in the respective line in Table II, application no. 14, column 5 or 8, or homologs or fragments thereof, and activates its transcription. A chimeric zinc finger protein can be constructed, which comprises a specific DNA-binding domain and an activation domain as e.g. the VP16 domain of Herpes Simplex virus. The specific binding domain can bind to the regulatory region of the gene encoding the protein as shown in the respective line in Table II, application no. 14, column 5 or 8, or homologs or fragments thereof. The expression of the chimeric transcription factor in a non-human organism, in particular in a plant, leads to a specific expression of the protein as shown in the respective line in Table II, application no. 14, column 5 or 8, or homologs or fragments thereof, see e.g. in WO01/52620, Oriz, Proc. Natl. Acad. Sci. USA 99, 13290 (2002), or Guan, Proc. Natl. Acad. Sci. USA 99, 13296 (2002).

[0124.1.7.14] to [0127.1.7.14] for the disclosure of these paragraphs see [0124.1.7.7] to [0127.1.7.7] above

Owing to the introduction of a gene or a plurality of genes conferring the expression of the nucleic acid molecule of the invention or the polypeptide of the invention, for example the nucleic acid construct mentioned below, or encoding the protein as shown in the respective line in Table II, application no. 14, column 5 or 8, or homologs or fragments thereof, into a non-human organism alone or in combination with other genes, it is possible not only to increase the biosynthetic flux towards the end product, but also to increase, modify or create de novo an advantageous, preferably novel metabolites composition in the non-human organism, e.g. an advantageous composition comprising a higher content of (from a viewpoint of nutritional physiology limited) beta-sitosterol, campesterol, or stigmasterol and if desired other steroids, and/or other metabolites, in free or bound form.

for the disclosure of this paragraph see [0129.1.7.7] above

Accordingly, in one embodiment, the process according to the invention relates to a process, which comprises:
(a) providing a non-human organism, preferably a microorganism, a plant cell, a plant tissue, a plant or a part thereof, more preferably a microorganism, a plant tissue, a plant or a part thereof;
(b) increasing the activity of a protein as shown in the respective line in Table II, application no. 14, column 3 or of a polypeptide being encoded by the respective nucleic acid molecule of the present invention and described below, e.g. conferring an increase of the fine chemical beta-sitosterol, campesterol, or stigmasterol, respectively, in the non-human organism, preferably in the microorganism, the plant cell, the plant tissue, the plant or a part thereof, more preferably a microorganism, a plant tissue, a plant or a part thereof, especially cytoplasmic or in an organelle, like plastids or mitochondria,
(c) growing the non-human organism, preferably the microorganism, the plant cell, the plant tissue, the plant or a part thereof, under conditions which permit the production of said chemical in the non-human organism, preferably the microorganism, the plant cell, the plant tissue, the plant or a part thereof; and
(d) if desired, recovering, optionally isolating, said fine chemical, in free and/or bound form, and, optionally further free and/or bound steroid, and/or other metabolites synthesized by the non-human organism, the microorganism, the plant cell, the plant tissue, the plant or a part thereof.

[0131.1.7.14] to [0133.1.7.14] for the disclosure of these paragraphs see [0131.1.7.7] to [0133.1.7.7] above

The non-human organism, in particular the microorganism, plant or part thereof, is advantageously grown in such a way that it is not only possible to recover, if desired to isolate the free or bound fine chemical or the free and bound fine chemical but as option it is also possible to produce, recover and, if desired to isolate, other free or/and bound vitamins and/or other metabolites.

The organisms are harvested in a manner known per se and appropriate for the particular organism. Microorganisms such as bacteria, mosses, yeasts and fungi or plant cells which are cultured in liquid media by fermentation may be removed, for example, by centrifugation, decanting or filtration. Plants are grown on solid media in a manner known per se and harvested accordingly.

Accordingly, the fine chemical, which is synthesized in the organism, in particular the microorganism, the plant or animal cell, the plant or animal tissue or the plant can be isolated if desired. Depending on the use of the fine chemical, different purities resulting from the purification may be advantageous as will be described herein below.

For example, after the growing phase, the organisms can be harvested. The organisms, its cells or a part thereof or the recovered, and if desired isolated, phytosterols, in particular beta-sitosterol, campesterol and/or stigmasterol, or esters, glycosides, triglycerides, lipids, oils and/or fats containing beta-sitosterol, campesterol and/or stigmasterol can then be processed further directly into foodstuffs or animal feeds or for other applications, for example according to the disclosures made in WO/2005/083093 or WO2005/012316, which are expressly incorporated herein by reference.

Transgenic plants which comprise the phytosterols such as beta-sitosterol, stigmasterol and/or campesterol synthesized in the process according to the invention can advantageously be marketed directly without there being any need for the phytosterols synthesized to be isolated. Plants for the process according to the invention are listed as meaning intact plants and all plant parts, plant organs or plant parts such as leaf, stem, seeds, root, tubers, progeny, anthers, fibers, root hairs, stalks, embryos, calli, cotelydons, petioles, harvested material, plant tissue, reproductive tissue and cell cultures which are derived from the actual transgenic plant and/or can be used for bringing about the transgenic plant. In this context, the seed comprises all parts of the seed such as the seed coats, epidermal cells, seed cells, endosperm or embryonic tissue. However, the fine chemical produced in the process according to the invention can also be isolated from the organisms, advantageously plants, in the form of their oils, fats, lipids containing beta-sitosterol and/or campesterol and/or in free form such as beta-sitosterol, stigmasterol and/or campestrerol. Phytosterols produced by this process can be obtained by harvesting the organisms, either from the crop in which they grow, or from the field. This can be done via pressing or extraction of the plant parts, preferably the plant seeds, as extracts, e.g. ether, alcohol, or other organic solvents or water containing extract. To increase the efficiency of extraction it is beneficial to clean, to temper and if necessary to hull and to flake the plant material expecially the progeny or the seeds. E.g the oils, fats, lipids, esters and/or free phytosterols, extracts, e.g. ether, alcohol, or other organic solvents or water containing extract which contain the phytosterols, in particular beta-sitosterol and/or campesterol produced according to the process of the invention can be obtained by what is known as cold beating or cold pressing without applying heat. To allow for greater ease of disruption of the plant parts, specifically the seeds, they are previously comminuted, steamed or roasted. The seeds, which have been pretreated in this manner can subsequently be pressed or extracted with solvents such as warm hexane. The solvent is subsequently removed. In the case of microorganisms, the latter are, after harvesting, for example extracted directly without further processing steps or else, after disruption, extracted via various methods with which the skilled worker is familiar. In this manner, more than 96% of the compounds produced in the process can be isolated. Thereafter, the resulting products are processed further, i.e. degummed and/or refined. In this process, substances such as the plant mucilages and suspended matter are first removed. What is known as desliming can be affected enzymatically or, for example, chemico-physically by addition of acid such as phosphoric acid.

Plant sterols (phytosterols) are by-products of traditional vegetable oil refining. The source may be commonly a blend of crude edible oils, consisting of soy bean oil or of other edible oils, e.g. corn, rapeseed, olive and palm oil in varying proportions. Hemp may also be a source of new oilseed, oil and food ingredients as well as Sea buckthorn (hippophae rhamnoides). The crude oil, which is obtained by pressing or solvent extraction, may undergoes a series of refining processes to remove solvents, lecithins, free fatty acids, color bodies, off-odors and off-flavors.

In one of these steps, the oil may be subjected to steam distillation at reduced pressure (deodorisation) and the resulting distillate contains the phytosterol fraction. From this fraction, fatty acids, lecithins and other compounds are removed by fractional distillation, ethanolysis/transesterification, distillation and crystallisation from a heptane solution, and the phytosterols are further purified by recrystallisation using food grade materials and good manufacturing practices. The extraction and purification steps are standard methods and similar to the procedures used traditionally by the food industry for the production of plant sterols. Phytosterol esters may be produced from the sterols using food grade vegetable oil-derived fatty acids or triglycerides and applying standard methods for esterification or transesterification commonly used in the fats and oils industry.

[0135.1.7.14] to [0139.1.7.14] for the disclosure of these paragraphs see [0135.1.7.7] to [0139.1.7.7] above

In another embodiment, the invention relates to a (isolated) nucleic acid molecule, which comprises a nucleic acid molecule selected from the group consisting of:
(a) a nucleic acid molecule encoding a polypeptide shown in Table II, application no. 14, column 5 or 8, preferably shown in Table II B, application no. 14, column 8, or a homolog or a fragment thereof;
(b) a nucleic acid molecule shown in Table I, application no. 14, column 5 or 8, preferably shown in Table I B, application no. 14, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 14, preferably in column 8 of Table II B, application no. 14;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 14, preferably shown in column 8 of Table I B, application no. 14, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 14;
(i) a nucleic acid molecule encoding a polypeptide having the activity represented by a respective protein as depicted in column 5 of Table II, application no. 14;
(j) a nucleic acid molecule comprising a polynucleotide which is obtained by amplifying a cDNA library or a genomic library using the primers in Table III, application no. 14, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j) ;
or a nucleic acid molecule comprising a sequence which is complementary thereto.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 14.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II application no. 14 and confers a generation or an increase of the respective fine chemical in a non-human organism or a part thereof as compared to a corresponding wild-type non-human organism or part thereof.

In another embodiment, the invention relates to a (isolated) nucleic acid molecule, which comprises a nucleic acid molecule selected from the group consisting of:
(a) a nucleic acid molecule encoding a polypeptide shown in Table II, column 5 or 8, application no. 14, preferably shown in Table II A, application no. 14, in column 5 or in Table II A, application no. 14, column 8 or in Table II B, application no. 14, column 8, or a homolog or a fragment thereof;
(b) a nucleic acid molecule shown in Table I, application no. 14, column 5 or 8, preferably shown in Table I A, application no. 14, in column 5 or in Table I A, application no. 14, column 8 or in Table I B, application no. 14, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 14, preferably shown in Table II A, application no. 14, in column 5 or in Table II A, application no. 14, column 8 or in Table II B, application no. 14, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 14, preferably shown in Table I A, application no. 14, in column 5 or in Table I A, application no. 14, column 8 or in Table I B column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 14;
(i) a nucleic acid molecule encoding a polypeptide having the activity represented by a respective protein as depicted in column 5 of Table II, application no. 14;
(j) a nucleic acid molecule comprising a polynucleotide which is obtained by amplifying a cDNA library or a genomic library using the primers in Table III, application no. 14, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt,500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto;
   whereby, preferably, the nucleic acid molecule according to (a), (b), (c), (d), (e), (f), (g), (h), (i), (j) or (k) distinguishes over the sequence depicted in Table I A and/or I B, application no. 14, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 14, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 14, column 5 or 8, or the coding region thereof, respectively. In a further embodiment the nucleic acid molecule does not encode the polypeptide sequence shown in Table II A and/or II B, application no. 14, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 14, column 5 or 8 and does not encode a protein of the sequence shown in Table II A and/or II B, application no. 14, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 14, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 14, column 5 or 8 but less than 100%, preferably less than 99.999%, 99.99%, 99.9%, 99%, 98%, 97%, 96% or 95% identical to the sequence shown in Table II A and/or II B, application no. 14, column 5 or 8.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 14.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II, application no. 14, and confers a generation or an increase of the respective fine chemical in a non-human organism or a part thereof as compared to a corresponding wild-type non-human organism or part thereof.

[0144.1.7.14] to [0155.1.7.14] for the disclosure of these paragraphs see [0144.1.7.7] to [0155.1.7.7] above

In a preferred embodiment a nucleic acid construct, for example an expression cassette, comprises upstream, i.e. at the 5′ end of the encoding sequence, a promoter and downstream, i.e. at the 3′ end, a polyadenylation signal and optionally other regulatory elements which are operably linked to the intervening encoding sequence with one of the nucleic acids as depicted in the respective line in Table I, application no. 14, column 5 or 8, preferably the coding region thereof, or a homolog or a fragment thereof. By an operable linkage is meant the sequential arrangement of promoter, encoding sequence, terminator, optionally other regulatory elements and optionally targeting sequences in such a way that each of the regulatory elements/targeting sequence can fulfill its function in the expression of the encoding sequence in due manner. In one embodiment the sequences preferred for operable linkage are targeting sequences for ensuring subcellular localization in organelles, like plastids or mitochondria. However, targeting sequences for ensuring subcellular localization in the endoplasmic reticulum (=ER), in the nucleus, in oil corpuscles or other compartments may also be employed as well as translation promoters such as the 5′ lead sequence in tobacco mosaic virus (Gallie et al., Nucl. Acids Res. 15, 8693 (1987)).

for the disclosure of this paragraph see [0157.1.7.7] above

In an embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 14, column 5 or 8, preferably shown in Table II A, application no. 14, column 5, or in Table II A, application no. 14, column 8, or in Table II B, application no. 14, column 8, or a homolog or a fragment thereof;
   (b) a nucleic acid molecule shown in Table I, application no. 14, column 5 or 8, preferably shown in Table I A, application no. 14, column 5, or in Table I A, application no. 14, column 8, or in Table I B, application no. 14, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 14, preferably shown in Table II A, application no. 14, column 5, or in Table II A, application no. 14, column 8, or in Table II B, application no. 14, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 14, preferably shown in Table I A, application no. 14, column 5, or in Table I A, application no. 14, column 8, or in Table I B, application no. 14, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 14;
   (i) a nucleic acid molecule encoding a polypeptide having the activity represented by a respective protein as depicted in column 5 of Table II, application no. 14;
   (j) a nucleic acid molecule comprising a polynucleotide which is obtained by amplifying a cDNA library or a genomic library using the primers in Table III, application no. 14, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto.
      and, optionally
3) nucleic acid molecule encoding a transit peptide, preferably encoding a plastidal transit peptide or a mitochondrial transit peptide;
   which are operable linked.
   (All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 14.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 14 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 14, in the same line as of the respective nucleic acid molecule according to 2) “plastidic” is depicted, or encoding a mitochondrial transit peptide, especially in case in column 6 of Table I, application no. 14, in the same line as of the respective nucleic acid molecule according to 2) “mitochondric” is depicted.

In another embodiment thereof the expression cassette does not comprise 3) a nucleic acid molecule encoding a transit peptide, especially in case in column 6 of Table I, application no. 14, in the same line as of the respective nucleic acid molecule according to 2) “non-targeted” is depicted.

In a further embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 14, column 5 or 8, preferably shown in Table II A, application no. 14, column 5, or in Table II A, application no. 14, column 8, or in Table II B, application no. 14, column 8, or a homolog or a fragment thereof;
   (b) a nucleic acid molecule shown in Table I, application no. 14, column 5 or 8, preferably shown in Table I A, application no. 14, column 5, or in Table I A, application no. 14, column 8, or in Table I B, application no. 14, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 14, preferably shown in Table II A, application no. 14, column 5, or in Table II A, application no. 14, column 8, or in Table II B, application no. 14, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 14, preferably shown in Table I A, application no. 14, column 5, or in Table I A, application no. 14, column 8,or in Table I B, application no. 14, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 14;
   (i) a nucleic acid molecule encoding a polypeptide having the activity represented by a respective protein as depicted in column 5 of Table II, application no. 14;
   (j) a nucleic acid molecule comprising a polynucleotide which is obtained by amplifying a cDNA library or a genomic library using the primers in Table III, application no. 14, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto; and, optionally
3) nucleic acid molecule encoding a transit peptide, preferably encoding a plastidal transit peptide or a mitochondrial transit peptide;
   which are operable linked and
   whereby, preferably, the nucleic acid molecule according to 2 (a), (b), (c), (d), (e), (f), (g), (h), (i), (j) or (k) distinguishes over the sequence depicted in Table I A and/or I B, application no. 14, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention according to 2) does not consist of the sequence shown in Table I A and/or I B, application no. 14, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 14, column 5 or 8, or the coding region thereof, respectively. In a further embodiment the nucleic acid molecule does not encode the polypeptide sequence shown in Table II A and/or II B, application no. 14, column 5 or 8 but homologs thereof.

Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 14, column 5 or 8 and does not encode a protein of the sequence shown in Table II A and/or II B, application no. 14, column 5 or 8. Accordingly, in one embodiment, the protein encoded by a sequence of a nucleic acid according to 2(a), (b), (c), (d), (e), (f), (g), (h), (i), (j) or (k) does not consist of the sequence shown in Table I A and/or I B, application no. 14, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 14, column 5 or 8 but less than 100%, 99.999%, 99.99%, 99.9%, 99%, 98%, 97%, 96% or 95% identical to the sequence shown in Table II A and/or II B, application no. 14, column 5 or 8.

(All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 14.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 14 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 14, in the same line as of the respective nucleic acid molecule according to 2) “plastidic” is depicted, or encoding a mitochondrial transit peptide, especially in case in column 6 of Table I, application no. 14, in the same line as of the respective nucleic acid molecule according to 2) “mitochondric” is depicted.

In another embodiment thereof the expression cassette does not comprise 3) a nucleic acid molecule encoding a transit peptide, especially in case in column 6 of Table I, application no. 14, in the same line as of the respective nucleic acid molecule according to 2) “non-targeted” is depicted.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 131003, or a homolog or fragment thereof, or a homolog thereof being depicted in Table I, application no. 14, column 8, in the same line as SEQ ID NO. 131003, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 131003 is depicted, plastidic is mentioned, or a mitochondrial transit peptide in case in Table I, application no. 14, in column 6 in the same line as SEQ ID NO. 131003 is depicted, mitochondric is mentioned; which is operable linked, too.

In another embodiment thereof the expression cassette does not comprise an additional transit peptide in case in Table I, application no. 14, in column 6 in the same line as SEQ ID NO. 131003 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17451, or a homolog or fragment thereof, or a homolog thereof being depicted in Table I, application no. 14, column 8, in the same line as SEQ ID NO. 17451, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 17451 is depicted, plastidic is mentioned, or a mitochondrial transit peptide in case in Table I, application no. 14, in column 6 in the same line as SEQ ID NO. 17451 is depicted, mitochondric is mentioned; which is operable linked, too.

In another embodiment thereof the expression cassette does not comprise an additional transit peptide in case in Table I, application no. 14, in column 6 in the same line as SEQ ID NO. 17451 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17968, or a homolog or fragment thereof, or a homolog thereof being depicted in Table I, application no. 14, column 8, in the same line as SEQ ID NO. 17968, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 17968 is depicted, plastidic is mentioned, or a mitochondrial transit peptide in case in Table I, application no. 14, in column 6 in the same line as SEQ ID NO. 17968 is depicted, mitochondric is mentioned; which is operable linked, too.

In another embodiment thereof the expression cassette does not comprise an additional transit peptide in case in Table I, application no. 14, in column 6 in the same line as SEQ ID NO. 17968 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 18869, or a homolog or fragment thereof, or a homolog thereof being depicted in Table I, application no. 14, column 8, in the same line as SEQ ID NO. 18869, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 18869 is depicted, plastidic is mentioned, or a mitochondrial transit peptide in case in Table I, application no. 14, in column 6 in the same line as SEQ ID NO. 18869 is depicted, mitochondric is mentioned; which is operable linked, too.

In another embodiment thereof the expression cassette does not comprise an additional transit peptide in case in Table I, application no. 14, in column 6 in the same line as SEQ ID NO. 18869 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 1061, or a homolog or fragment thereof, or a homolog thereof being depicted in Table I, application no. 14, column 8, in the same line as SEQ ID NO. 1061, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 1061 is depicted, plastidic is mentioned, or a mitochondrial transit peptide in case in Table I, application no. 14, in column 6 in the same line as SEQ ID NO. 1061 is depicted, mitochondric is mentioned; which is operable linked, too.

In another embodiment thereof the expression cassette does not comprise an additional transit peptide in case in Table I, application no. 14, in column 6 in the same line as SEQ ID NO. 1061 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 19419, or a homolog or fragment thereof, or a homolog thereof being depicted in Table I, application no. 14, column 8, in the same line as SEQ ID NO. 19419, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 19419 is depicted, plastidic is mentioned, or a mitochondrial transit peptide in case in Table I, application no. 14, in column 6 in the same line as SEQ ID NO. 19419 is depicted, mitochondric is mentioned; which is operable linked, too.

In another embodiment thereof the expression cassette does not comprise an additional transit peptide in case in Table I, application no. 14, in column 6 in the same line as SEQ ID NO. 19419 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 21902, or a homolog or fragment thereof, or a homolog thereof being depicted in Table I, application no. 14, column 8, in the same line as SEQ ID NO. 21902, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 21902 is depicted, plastidic is mentioned, or a mitochondrial transit peptide in case in Table I, application no. 14, in column 6 in the same line as SEQ ID NO. 21902 is depicted, mitochondric is mentioned; which is operable linked, too.

In another embodiment thereof the expression cassette does not comprise an additional transit peptide in case in Table I, application no. 14, in column 6 in the same line as SEQ ID NO. 21902 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 109717, or a homolog or fragment thereof, or a homolog thereof being depicted in Table I, application no. 14, column 8, in the same line as SEQ ID NO. 109717, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 109717 is depicted, plastidic is mentioned, or a mitochondrial transit peptide in case in Table I, application no. 14, in column 6 in the same line as SEQ ID NO. 109717 is depicted, mitochondric is mentioned; which is operable linked, too.

In another embodiment thereof the expression cassette does not comprise an additional transit peptide in case in Table I, application no. 14, in column 6 in the same line as SEQ ID NO. 109717 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 131208, or a homolog or fragment thereof, or a homolog thereof being depicted in Table I, application no. 14, column 8, in the same line as SEQ ID NO. 131208, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 131208 is depicted, plastidic is mentioned, or a mitochondrial transit peptide in case in Table I, application no. 14, in column 6 in the same line as SEQ ID NO. 131208 is depicted, mitochondric is mentioned; which is operable linked, too.

In another embodiment thereof the expression cassette does not comprise an additional transit peptide in case in Table I, application no. 14, in column 6 in the same line as SEQ ID NO. 131208 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 23482, or a homolog or fragment thereof, or a homolog thereof being depicted in Table I, application no. 14, column 8, in the same line as SEQ ID NO. 23482, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 23482 is depicted, plastidic is mentioned, or a mitochondrial transit peptide in case in Table I, application no. 14, in column 6 in the same line as SEQ ID NO. 23482 is depicted, mitochondric is mentioned; which is operable linked, too.

In another embodiment thereof the expression cassette does not comprise an additional transit peptide in case in Table I, application no. 14, in column 6 in the same line as SEQ ID NO. 23482 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68849, or a homolog or fragment thereof, or a homolog thereof being depicted in Table I, application no. 14, column 8, in the same line as SEQ ID NO. 68849, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 68849 is depicted, plastidic is mentioned, or a mitochondrial transit peptide in case in Table I, application no. 14, in column 6 in the same line as SEQ ID NO. 68849 is depicted, mitochondric is mentioned; which is operable linked, too.

In another embodiment thereof the expression cassette does not comprise an additional transit peptide in case in Table I, application no. 14, in column 6 in the same line as SEQ ID NO. 68849 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4348, or a homolog or fragment thereof, or a homolog thereof being depicted in Table I, application no. 14, column 8, in the same line as SEQ ID NO. 4348, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 4348 is depicted, plastidic is mentioned, or a mitochondrial transit peptide in case in Table I, application no. 14, in column 6 in the same line as SEQ ID NO. 4348 is depicted, mitochondric is mentioned; which is operable linked, too.

In another embodiment thereof the expression cassette does not comprise an additional transit peptide in case in Table I, application no. 14, in column 6 in the same line as SEQ ID NO. 4348 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 5318, or a homolog or fragment thereof, or a homolog thereof being depicted in Table I, application no. 14, column 8, in the same line as SEQ ID NO. 5318, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 5318 is depicted, plastidic is mentioned, or a mitochondrial transit peptide in case in Table I, application no. 14, in column 6 in the same line as SEQ ID NO. 5318 is depicted, mitochondric is mentioned; which is operable linked, too.

In another embodiment thereof the expression cassette does not comprise an additional transit peptide in case in Table I, application no. 14, in column 6 in the same line as SEQ ID NO. 5318 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 25498, or a homolog or fragment thereof, or a homolog thereof being depicted in Table I, application no. 14, column 8, in the same line as SEQ ID NO. 25498, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 25498 is depicted, plastidic is mentioned, or a mitochondrial transit peptide in case in Table I, application no. 14, in column 6 in the same line as SEQ ID NO. 25498 is depicted, mitochondric is mentioned; which is operable linked, too.

In another embodiment thereof the expression cassette does not comprise an additional transit peptide in case in Table I, application no. 14, in column 6 in the same line as SEQ ID NO. 25498 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 122803, or a homolog or fragment thereof, or a homolog thereof being depicted in Table I, application no. 14, column 8, in the same line as SEQ ID NO. 122803, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 122803 is depicted, plastidic is mentioned, or a mitochondrial transit peptide in case in Table I, application no. 14, in column 6 in the same line as SEQ ID NO. 122803 is depicted, mitochondric is mentioned; which is operable linked, too.

In another embodiment thereof the expression cassette does not comprise an additional transit peptide in case in Table I, application no. 14, in column 6 in the same line as SEQ ID NO. 122803 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 26196, or a homolog or fragment thereof, or a homolog thereof being depicted in Table I, application no. 14, column 8, in the same line as SEQ ID NO. 26196, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 26196 is depicted, plastidic is mentioned, or a mitochondrial transit peptide in case in Table I, application no. 14, in column 6 in the same line as SEQ ID NO. 26196 is depicted, mitochondric is mentioned; which is operable linked, too.

In another embodiment thereof the expression cassette does not comprise an additional transit peptide in case in Table I, application no. 14, in column 6 in the same line as SEQ ID NO. 26196 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6075, or a homolog or fragment thereof, or a homolog thereof being depicted in Table I, application no. 14, column 8, in the same line as SEQ ID NO. 6075, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 6075 is depicted, plastidic is mentioned, or a mitochondrial transit peptide in case in Table I, application no. 14, in column 6 in the same line as SEQ ID NO. 6075 is depicted, mitochondric is mentioned; which is operable linked, too.

In another embodiment thereof the expression cassette does not comprise an additional transit peptide in case in Table I, application no. 14, in column 6 in the same line as SEQ ID NO. 6075 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 29500, or a homolog or fragment thereof, or a homolog thereof being depicted in Table I, application no. 14, column 8, in the same line as SEQ ID NO. 29500, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 29500 is depicted, plastidic is mentioned, or a mitochondrial transit peptide in case in Table I, application no. 14, in column 6 in the same line as SEQ ID NO. 29500 is depicted, mitochondric is mentioned; which is operable linked, too.

In another embodiment thereof the expression cassette does not comprise an additional transit peptide in case in Table I, application no. 14, in column 6 in the same line as SEQ ID NO. 29500 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 31026, or a homolog or fragment thereof, or a homolog thereof being depicted in Table I, application no. 14, column 8, in the same line as SEQ ID NO. 31026, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 31026 is depicted, plastidic is mentioned, or a mitochondrial transit peptide in case in Table I, application no. 14, in column 6 in the same line as SEQ ID NO. 31026 is depicted, mitochondric is mentioned; which is operable linked, too.

In another embodiment thereof the expression cassette does not comprise an additional transit peptide in case in Table I, application no. 14, in column 6 in the same line as SEQ ID NO. 31026 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 114231, or a homolog or fragment thereof, or a homolog thereof being depicted in Table I, application no. 14, column 8, in the same line as SEQ ID NO. 114231, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 114231 is depicted, plastidic is mentioned, or a mitochondrial transit peptide in case in Table I, application no. 14, in column 6 in the same line as SEQ ID NO. 114231 is depicted, mitochondric is mentioned; which is operable linked, too.

In another embodiment thereof the expression cassette does not comprise an additional transit peptide in case in Table I, application no. 14, in column 6 in the same line as SEQ ID NO. 114231 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6510, or a homolog or fragment thereof, or a homolog thereof being depicted in Table I, application no. 14, column 8, in the same line as SEQ ID NO. 6510, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 6510 is depicted, plastidic is mentioned, or a mitochondrial transit peptide in case in Table I, application no. 14, in column 6 in the same line as SEQ ID NO. 6510 is depicted, mitochondric is mentioned; which is operable linked, too.

In another embodiment thereof the expression cassette does not comprise an additional transit peptide in case in Table I, application no. 14, in column 6 in the same line as SEQ ID NO. 6510 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 34889, or a homolog or fragment thereof, or a homolog thereof being depicted in Table I, application no. 14, column 8, in the same line as SEQ ID NO. 34889, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 34889 is depicted, plastidic is mentioned, or a mitochondrial transit peptide in case in Table I, application no. 14, in column 6 in the same line as SEQ ID NO. 34889 is depicted, mitochondric is mentioned; which is operable linked, too.

In another embodiment thereof the expression cassette does not comprise an additional transit peptide in case in Table I, application no. 14, in column 6 in the same line as SEQ ID NO. 34889 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 35204, or a homolog or fragment thereof, or a homolog thereof being depicted in Table I, application no. 14, column 8, in the same line as SEQ ID NO. 35204, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 35204 is depicted, plastidic is mentioned, or a mitochondrial transit peptide in case in Table I, application no. 14, in column 6 in the same line as SEQ ID NO. 35204 is depicted, mitochondric is mentioned; which is operable linked, too.

In another embodiment thereof the expression cassette does not comprise an additional transit peptide in case in Table I, application no. 14, in column 6 in the same line as SEQ ID NO. 35204 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 35482, or a homolog or fragment thereof, or a homolog thereof being depicted in Table I, application no. 14, column 8, in the same line as SEQ ID NO. 35482, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 35482 is depicted, plastidic is mentioned, or a mitochondrial transit peptide in case in Table I, application no. 14, in column 6 in the same line as SEQ ID NO. 35482 is depicted, mitochondric is mentioned; which is operable linked, too.

In another embodiment thereof the expression cassette does not comprise an additional transit peptide in case in Table I, application no. 14, in column 6 in the same line as SEQ ID NO. 35482 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7333, or a homolog or fragment thereof, or a homolog thereof being depicted in Table I, application no. 14, column 8, in the same line as SEQ ID NO. 7333, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 7333 is depicted, plastidic is mentioned, or a mitochondrial transit peptide in case in Table I, application no. 14, in column 6 in the same line as SEQ ID NO. 7333 is depicted, mitochondric is mentioned; which is operable linked, too.

In another embodiment thereof the expression cassette does not comprise an additional transit peptide in case in Table I, application no. 14, in column 6 in the same line as SEQ ID NO. 7333 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7686, or a homolog or fragment thereof, or a homolog thereof being depicted in Table I, application no. 14, column 8, in the same line as SEQ ID NO. 7686, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 7686 is depicted, plastidic is mentioned, or a mitochondrial transit peptide in case in Table I, application no. 14, in column 6 in the same line as SEQ ID NO. 7686 is depicted, mitochondric is mentioned; which is operable linked, too.

In another embodiment thereof the expression cassette does not comprise an additional transit peptide in case in Table I, application no. 14, in column 6 in the same line as SEQ ID NO. 7686 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 131359, or a homolog or fragment thereof, or a homolog thereof being depicted in Table I, application no. 14, column 8, in the same line as SEQ ID NO. 131359, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 131359 is depicted, plastidic is mentioned, or a mitochondrial transit peptide in case in Table I, application no. 14, in column 6 in the same line as SEQ ID NO. 131359 is depicted, mitochondric is mentioned; which is operable linked, too.

In another embodiment thereof the expression cassette does not comprise an additional transit peptide in case in Table I, application no. 14, in column 6 in the same line as SEQ ID NO. 131359 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 76032, or a homolog or fragment thereof, or a homolog thereof being depicted in Table I, application no. 14, column 8, in the same line as SEQ ID NO. 76032, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 76032 is depicted, plastidic is mentioned, or a mitochondrial transit peptide in case in Table I, application no. 14, in column 6 in the same line as SEQ ID NO. 76032 is depicted, mitochondric is mentioned; which is operable linked, too.

In another embodiment thereof the expression cassette does not comprise an additional transit peptide in case in Table I, application no. 14, in column 6 in the same line as SEQ ID NO. 76032 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 37400, or a homolog or fragment thereof, or a homolog thereof being depicted in Table I, application no. 14, column 8, in the same line as SEQ ID NO. 37400, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 37400 is depicted, plastidic is mentioned, or a mitochondrial transit peptide in case in Table I, application no. 14, in column 6 in the same line as SEQ ID NO. 37400 is depicted, mitochondric is mentioned; which is operable linked, too.

In another embodiment thereof the expression cassette does not comprise an additional transit peptide in case in Table I, application no. 14, in column 6 in the same line as SEQ ID NO. 37400 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 131489, or a homolog or fragment thereof, or a homolog thereof being depicted in Table I, application no. 14, column 8, in the same line as SEQ ID NO. 131489, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 131489 is depicted, plastidic is mentioned, or a mitochondrial transit peptide in case in Table I, application no. 14, in column 6 in the same line as SEQ ID NO. 131489 is depicted, mitochondric is mentioned; which is operable linked, too.

In another embodiment thereof the expression cassette does not comprise an additional transit peptide in case in Table I, application no. 14, in column 6 in the same line as SEQ ID NO. 131489 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 131531, or a homolog or fragment thereof, or a homolog thereof being depicted in Table I, application no. 14, column 8, in the same line as SEQ ID NO. 131531, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 131531 is depicted, plastidic is mentioned, or a mitochondrial transit peptide in case in Table I, application no. 14, in column 6 in the same line as SEQ ID NO. 131531 is depicted, mitochondric is mentioned; which is operable linked, too.

In another embodiment thereof the expression cassette does not comprise an additional transit peptide in case in Table I, application no. 14, in column 6 in the same line as SEQ ID NO. 131531 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38266, or a homolog or fragment thereof, or a homolog thereof being depicted in Table I, application no. 14, column 8, in the same line as SEQ ID NO. 38266, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 38266 is depicted, plastidic is mentioned, or a mitochondrial transit peptide in case in Table I, application no. 14, in column 6 in the same line as SEQ ID NO. 38266 is depicted, mitochondric is mentioned; which is operable linked, too.

In another embodiment thereof the expression cassette does not comprise an additional transit peptide in case in Table I, application no. 14, in column 6 in the same line as SEQ ID NO. 38266 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38300, or a homolog or fragment thereof, or a homolog thereof being depicted in Table I, application no. 14, column 8, in the same line as SEQ ID NO. 38300, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 38300 is depicted, plastidic is mentioned, or a mitochondrial transit peptide in case in Table I, application no. 14, in column 6 in the same line as SEQ ID NO. 38300 is depicted, mitochondric is mentioned; which is operable linked, too.

In another embodiment thereof the expression cassette does not comprise an additional transit peptide in case in Table I, application no. 14, in column 6 in the same line as SEQ ID NO. 38300 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 131539, or a homolog or fragment thereof, or a homolog thereof being depicted in Table I, application no. 14, column 8, in the same line as SEQ ID NO. 131539, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 131539 is depicted, plastidic is mentioned, or a mitochondrial transit peptide in case in Table I, application no. 14, in column 6 in the same line as SEQ ID NO. 131539 is depicted, mitochondric is mentioned; which is operable linked, too.

In another embodiment thereof the expression cassette does not comprise an additional transit peptide in case in Table I, application no. 14, in column 6 in the same line as SEQ ID NO. 131539 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 131799, or a homolog or fragment thereof, or a homolog thereof being depicted in Table I, application no. 14, column 8, in the same line as SEQ ID NO. 131799, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 131799 is depicted, plastidic is mentioned, or a mitochondrial transit peptide in case in Table I, application no. 14, in column 6 in the same line as SEQ ID NO. 131799 is depicted, mitochondric is mentioned; which is operable linked, too.

In another embodiment thereof the expression cassette does not comprise an additional transit peptide in case in Table I, application no. 14, in column 6 in the same line as SEQ ID NO. 131799 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39002, or a homolog or fragment thereof, or a homolog thereof being depicted in Table I, application no. 14, column 8, in the same line as SEQ ID NO. 39002, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 39002 is depicted, plastidic is mentioned, or a mitochondrial transit peptide in case in Table I, application no. 14, in column 6 in the same line as SEQ ID NO. 39002 is depicted, mitochondric is mentioned; which is operable linked, too.

In another embodiment thereof the expression cassette does not comprise an additional transit peptide in case in Table I, application no. 14, in column 6 in the same line as SEQ ID NO. 39002 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 131839, or a homolog or fragment thereof, or a homolog thereof being depicted in Table I, application no. 14, column 8, in the same line as SEQ ID NO. 131839, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 131839 is depicted, plastidic is mentioned, or a mitochondrial transit peptide in case in Table I, application no. 14, in column 6 in the same line as SEQ ID NO. 131839 is depicted, mitochondric is mentioned; which is operable linked, too.

In another embodiment thereof the expression cassette does not comprise an additional transit peptide in case in Table I, application no. 14, in column 6 in the same line as SEQ ID NO. 131839 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 132086, or a homolog or fragment thereof, or a homolog thereof being depicted in Table I, application no. 14, column 8, in the same line as SEQ ID NO. 132086, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 132086 is depicted, plastidic is mentioned, or a mitochondrial transit peptide in case in Table I, application no. 14, in column 6 in the same line as SEQ ID NO. 132086 is depicted, mitochondric is mentioned; which is operable linked, too.

In another embodiment thereof the expression cassette does not comprise an additional transit peptide in case in Table I, application no. 14, in column 6 in the same line as SEQ ID NO. 132086 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 132695, or a homolog or fragment thereof, or a homolog thereof being depicted in Table I, application no. 14, column 8, in the same line as SEQ ID NO. 132695, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 132695 is depicted, plastidic is mentioned, or a mitochondrial transit peptide in case in Table I, application no. 14, in column 6 in the same line as SEQ ID NO. 132695 is depicted, mitochondric is mentioned; which is operable linked, too.

In another embodiment thereof the expression cassette does not comprise an additional transit peptide in case in Table I, application no. 14, in column 6 in the same line as SEQ ID NO. 132695 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39237, or a homolog or fragment thereof, or a homolog thereof being depicted in Table I, application no. 14, column 8, in the same line as SEQ ID NO. 39237, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 39237 is depicted, plastidic is mentioned, or a mitochondrial transit peptide in case in Table I, application no. 14, in column 6 in the same line as SEQ ID NO. 39237 is depicted, mitochondric is mentioned; which is operable linked, too.

In another embodiment thereof the expression cassette does not comprise an additional transit peptide in case in Table I, application no. 14, in column 6 in the same line as SEQ ID NO. 39237 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40329, or a homolog or fragment thereof, or a homolog thereof being depicted in Table I, application no. 14, column 8, in the same line as SEQ ID NO. 40329, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 40329 is depicted, plastidic is mentioned, or a mitochondrial transit peptide in case in Table I, application no. 14, in column 6 in the same line as SEQ ID NO. 40329 is depicted, mitochondric is mentioned; which is operable linked, too.

In another embodiment thereof the expression cassette does not comprise an additional transit peptide in case in Table I, application no. 14, in column 6 in the same line as SEQ ID NO. 40329 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 132706, or a homolog or fragment thereof, or a homolog thereof being depicted in Table I, application no. 14, column 8, in the same line as SEQ ID NO. 132706, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 132706 is depicted, plastidic is mentioned, or a mitochondrial transit peptide in case in Table I, application no. 14, in column 6 in the same line as SEQ ID NO. 132706 is depicted, mitochondric is mentioned; which is operable linked, too.

In another embodiment thereof the expression cassette does not comprise an additional transit peptide in case in Table I, application no. 14, in column 6 in the same line as SEQ ID NO. 132706 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 9167, or a homolog or fragment thereof, or a homolog thereof being depicted in Table I, application no. 14, column 8, in the same line as SEQ ID NO. 9167, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 9167 is depicted, plastidic is mentioned, or a mitochondrial transit peptide in case in Table I, application no. 14, in column 6 in the same line as SEQ ID NO. 9167 is depicted, mitochondric is mentioned; which is operable linked, too.

In another embodiment thereof the expression cassette does not comprise an additional transit peptide in case in Table I, application no. 14, in column 6 in the same line as SEQ ID NO. 9167 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 99898, or a homolog or fragment thereof, or a homolog thereof being depicted in Table I, application no. 14, column 8, in the same line as SEQ ID NO. 99898, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 99898 is depicted, plastidic is mentioned, or a mitochondrial transit peptide in case in Table I, application no. 14, in column 6 in the same line as SEQ ID NO. 99898 is depicted, mitochondric is mentioned; which is operable linked, too.

In another embodiment thereof the expression cassette does not comprise an additional transit peptide in case in Table I, application no. 14, in column 6 in the same line as SEQ ID NO. 99898 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 125226, or a homolog or fragment thereof, or a homolog thereof being depicted in Table I, application no. 14, column 8, in the same line as SEQ ID NO. 125226, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 125226 is depicted, plastidic is mentioned, or a mitochondrial transit peptide in case in Table I, application no. 14, in column 6 in the same line as SEQ ID NO. 125226 is depicted, mitochondric is mentioned; which is operable linked, too.

In another embodiment thereof the expression cassette does not comprise an additional transit peptide in case in Table I, application no. 14, in column 6 in the same line as SEQ ID NO. 125226 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 80756, or a homolog or fragment thereof, or a homolog thereof being depicted in Table I, application no. 14, column 8, in the same line as SEQ ID NO. 80756, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 80756 is depicted, plastidic is mentioned, or a mitochondrial transit peptide in case in Table I, application no. 14, in column 6 in the same line as SEQ ID NO. 80756 is depicted, mitochondric is mentioned; which is operable linked, too.

In another embodiment thereof the expression cassette does not comprise an additional transit peptide in case in Table I, application no. 14, in column 6 in the same line as SEQ ID NO. 80756 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 132821, or a homolog or fragment thereof, or a homolog thereof being depicted in Table I, application no. 14, column 8, in the same line as SEQ ID NO. 132821, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 132821 is depicted, plastidic is mentioned, or a mitochondrial transit peptide in case in Table I, application no. 14, in column 6 in the same line as SEQ ID NO. 132821 is depicted, mitochondric is mentioned; which is operable linked, too.

In another embodiment thereof the expression cassette does not comprise an additional transit peptide in case in Table I, application no. 14, in column 6 in the same line as SEQ ID NO. 132821 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115864, or a homolog or fragment thereof, or a homolog thereof being depicted in Table I, application no. 14, column 8, in the same line as SEQ ID NO. 115864, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 115864 is depicted, plastidic is mentioned, or a mitochondrial transit peptide in case in Table I, application no. 14, in column 6 in the same line as SEQ ID NO. 115864 is depicted, mitochondric is mentioned; which is operable linked, too.

In another embodiment thereof the expression cassette does not comprise an additional transit peptide in case in Table I, application no. 14, in column 6 in the same line as SEQ ID NO. 115864 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 42477, or a homolog or fragment thereof, or a homolog thereof being depicted in Table I, application no. 14, column 8, in the same line as SEQ ID NO. 42477, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 42477 is depicted, plastidic is mentioned, or a mitochondrial transit peptide in case in Table I, application no. 14, in column 6 in the same line as SEQ ID NO. 42477 is depicted, mitochondric is mentioned; which is operable linked, too.

In another embodiment thereof the expression cassette does not comprise an additional transit peptide in case in Table I, application no. 14, in column 6 in the same line as SEQ ID NO. 42477 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 42502, or a homolog or fragment thereof, or a homolog thereof being depicted in Table I, application no. 14, column 8, in the same line as SEQ ID NO. 42502, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 42502 is depicted, plastidic is mentioned, or a mitochondrial transit peptide in case in Table I, application no. 14, in column 6 in the same line as SEQ ID NO. 42502 is depicted, mitochondric is mentioned; which is operable linked, too.

In another embodiment thereof the expression cassette does not comprise an additional transit peptide in case in Table I, application no. 14, in column 6 in the same line as SEQ ID NO. 42502 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 42600, or a homolog or fragment thereof, or a homolog thereof being depicted in Table I, application no. 14, column 8, in the same line as SEQ ID NO. 42600, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 42600 is depicted, plastidic is mentioned, or a mitochondrial transit peptide in case in Table I, application no. 14, in column 6 in the same line as SEQ ID NO. 42600 is depicted, mitochondric is mentioned; which is operable linked, too.

In another embodiment thereof the expression cassette does not comprise an additional transit peptide in case in Table I, application no. 14, in column 6 in the same line as SEQ ID NO. 42600 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 44372, or a homolog or fragment thereof, or a homolog thereof being depicted in Table I, application no. 14, column 8, in the same line as SEQ ID NO. 44372, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 44372 is depicted, plastidic is mentioned, or a mitochondrial transit peptide in case in Table I, application no. 14, in column 6 in the same line as SEQ ID NO. 44372 is depicted, mitochondric is mentioned; which is operable linked, too.

In another embodiment thereof the expression cassette does not comprise an additional transit peptide in case in Table I, application no. 14, in column 6 in the same line as SEQ ID NO. 44372 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 10740, or a homolog or fragment thereof, or a homolog thereof being depicted in Table I, application no. 14, column 8, in the same line as SEQ ID NO. 10740, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 10740 is depicted, plastidic is mentioned, or a mitochondrial transit peptide in case in Table I, application no. 14, in column 6 in the same line as SEQ ID NO. 10740 is depicted, mitochondric is mentioned; which is operable linked, too.

In another embodiment thereof the expression cassette does not comprise an additional transit peptide in case in Table I, application no. 14, in column 6 in the same line as SEQ ID NO. 10740 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 126975, or a homolog or fragment thereof, or a homolog thereof being depicted in Table I, application no. 14, column 8, in the same line as SEQ ID NO. 126975, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 126975 is depicted, plastidic is mentioned, or a mitochondrial transit peptide in case in Table I, application no. 14, in column 6 in the same line as SEQ ID NO. 126975 is depicted, mitochondric is mentioned; which is operable linked, too.

In another embodiment thereof the expression cassette does not comprise an additional transit peptide in case in Table I, application no. 14, in column 6 in the same line as SEQ ID NO. 126975 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 10811, or a homolog or fragment thereof, or a homolog thereof being depicted in Table I, application no. 14, column 8, in the same line as SEQ ID NO. 10811, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 10811 is depicted, plastidic is mentioned, or a mitochondrial transit peptide in case in Table I, application no. 14, in column 6 in the same line as SEQ ID NO. 10811 is depicted, mitochondric is mentioned; which is operable linked, too.

In another embodiment thereof the expression cassette does not comprise an additional transit peptide in case in Table I, application no. 14, in column 6 in the same line as SEQ ID NO. 10811 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 46751, or a homolog or fragment thereof, or a homolog thereof being depicted in Table I, application no. 14, column 8, in the same line as SEQ ID NO. 46751, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 46751 is depicted, plastidic is mentioned, or a mitochondrial transit peptide in case in Table I, application no. 14, in column 6 in the same line as SEQ ID NO. 46751 is depicted, mitochondric is mentioned; which is operable linked, too.

In another embodiment thereof the expression cassette does not comprise an additional transit peptide in case in Table I, application no. 14, in column 6 in the same line as SEQ ID NO. 46751 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 46850, or a homolog or fragment thereof, or a homolog thereof being depicted in Table I, application no. 14, column 8, in the same line as SEQ ID NO. 46850, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 46850 is depicted, plastidic is mentioned, or a mitochondrial transit peptide in case in Table I, application no. 14, in column 6 in the same line as SEQ ID NO. 46850 is depicted, mitochondric is mentioned; which is operable linked, too.

In another embodiment thereof the expression cassette does not comprise an additional transit peptide in case in Table I, application no. 14, in column 6 in the same line as SEQ ID NO. 46850 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 127103, or a homolog or fragment thereof, or a homolog thereof being depicted in Table I, application no. 14, column 8, in the same line as SEQ ID NO. 127103, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 127103 is depicted, plastidic is mentioned, or a mitochondrial transit peptide in case in Table I, application no. 14, in column 6 in the same line as SEQ ID NO. 127103 is depicted, mitochondric is mentioned; which is operable linked, too.

In another embodiment thereof the expression cassette does not comprise an additional transit peptide in case in Table I, application no. 14, in column 6 in the same line as SEQ ID NO. 127103 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 132874, or a homolog or fragment thereof, or a homolog thereof being depicted in Table I, application no. 14, column 8, in the same line as SEQ ID NO. 132874, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 132874 is depicted, plastidic is mentioned, or a mitochondrial transit peptide in case in Table I, application no. 14, in column 6 in the same line as SEQ ID NO. 132874 is depicted, mitochondric is mentioned; which is operable linked, too.

In another embodiment thereof the expression cassette does not comprise an additional transit peptide in case in Table I, application no. 14, in column 6 in the same line as SEQ ID NO. 132874 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 53878, or a homolog or fragment thereof, or a homolog thereof being depicted in Table I, application no. 14, column 8, in the same line as SEQ ID NO. 53878, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 53878 is depicted, plastidic is mentioned, or a mitochondrial transit peptide in case in Table I, application no. 14, in column 6 in the same line as SEQ ID NO. 53878 is depicted, mitochondric is mentioned; which is operable linked, too.

In another embodiment thereof the expression cassette does not comprise an additional transit peptide in case in Table I, application no. 14, in column 6 in the same line as SEQ ID NO. 53878 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 132938, or a homolog or fragment thereof, or a homolog thereof being depicted in Table I, application no. 14, column 8, in the same line as SEQ ID NO. 132938, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 132938 is depicted, plastidic is mentioned, or a mitochondrial transit peptide in case in Table I, application no. 14, in column 6 in the same line as SEQ ID NO. 132938 is depicted, mitochondric is mentioned; which is operable linked, too.

In another embodiment thereof the expression cassette does not comprise an additional transit peptide in case in Table I, application no. 14, in column 6 in the same line as SEQ ID NO. 132938 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 85743, or a homolog or fragment thereof, or a homolog thereof being depicted in Table I, application no. 14, column 8, in the same line as SEQ ID NO. 85743, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 85743 is depicted, plastidic is mentioned, or a mitochondrial transit peptide in case in Table I, application no. 14, in column 6 in the same line as SEQ ID NO. 85743 is depicted, mitochondric is mentioned; which is operable linked, too.

In another embodiment thereof the expression cassette does not comprise an additional transit peptide in case in Table I, application no. 14, in column 6 in the same line as SEQ ID NO. 85743 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 55379, or a homolog or fragment thereof, or a homolog thereof being depicted in Table I, application no. 14, column 8, in the same line as SEQ ID NO. 55379, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 55379 is depicted, plastidic is mentioned, or a mitochondrial transit peptide in case in Table I, application no. 14, in column 6 in the same line as SEQ ID NO. 55379 is depicted, mitochondric is mentioned; which is operable linked, too.

In another embodiment thereof the expression cassette does not comprise an additional transit peptide in case in Table I, application no. 14, in column 6 in the same line as SEQ ID NO. 55379 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 86447, or a homolog or fragment thereof, or a homolog thereof being depicted in Table I, application no. 14, column 8, in the same line as SEQ ID NO. 86447, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 86447 is depicted, plastidic is mentioned, or a mitochondrial transit peptide in case in Table I, application no. 14, in column 6 in the same line as SEQ ID NO. 86447 is depicted, mitochondric is mentioned; which is operable linked, too.

In another embodiment thereof the expression cassette does not comprise an additional transit peptide in case in Table I, application no. 14, in column 6 in the same line as SEQ ID NO. 86447 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 55385, or a homolog or fragment thereof, or a homolog thereof being depicted in Table I, application no. 14, column 8, in the same line as SEQ ID NO. 55385, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 55385 is depicted, plastidic is mentioned, or a mitochondrial transit peptide in case in Table I, application no. 14, in column 6 in the same line as SEQ ID NO. 55385 is depicted, mitochondric is mentioned; which is operable linked, too.

In another embodiment thereof the expression cassette does not comprise an additional transit peptide in case in Table I, application no. 14, in column 6 in the same line as SEQ ID NO. 55385 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 11990, or a homolog or fragment thereof, or a homolog thereof being depicted in Table I, application no. 14, column 8, in the same line as SEQ ID NO. 11990, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 11990 is depicted, plastidic is mentioned, or a mitochondrial transit peptide in case in Table I, application no. 14, in column 6 in the same line as SEQ ID NO. 11990 is depicted, mitochondric is mentioned; which is operable linked, too.

In another embodiment thereof the expression cassette does not comprise an additional transit peptide in case in Table I, application no. 14, in column 6 in the same line as SEQ ID NO. 11990 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 119222, or a homolog or fragment thereof, or a homolog thereof being depicted in Table I, application no. 14, column 8, in the same line as SEQ ID NO. 119222, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 119222 is depicted, plastidic is mentioned, or a mitochondrial transit peptide in case in Table I, application no. 14, in column 6 in the same line as SEQ ID NO. 119222 is depicted, mitochondric is mentioned; which is operable linked, too.

In another embodiment thereof the expression cassette does not comprise an additional transit peptide in case in Table I, application no. 14, in column 6 in the same line as SEQ ID NO. 119222 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 87655, or a homolog or fragment thereof, or a homolog thereof being depicted in Table I, application no. 14, column 8, in the same line as SEQ ID NO. 87655, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 87655 is depicted, plastidic is mentioned, or a mitochondrial transit peptide in case in Table I, application no. 14, in column 6 in the same line as SEQ ID NO. 87655 is depicted, mitochondric is mentioned; which is operable linked, too.

In another embodiment thereof the expression cassette does not comprise an additional transit peptide in case in Table I, application no. 14, in column 6 in the same line as SEQ ID NO. 87655 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 61723, or a homolog or fragment thereof, or a homolog thereof being depicted in Table I, application no. 14, column 8, in the same line as SEQ ID NO. 61723, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 61723 is depicted, plastidic is mentioned, or a mitochondrial transit peptide in case in Table I, application no. 14, in column 6 in the same line as SEQ ID NO. 61723 is depicted, mitochondric is mentioned; which is operable linked, too.

In another embodiment thereof the expression cassette does not comprise an additional transit peptide in case in Table I, application no. 14, in column 6 in the same line as SEQ ID NO. 61723 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 62079, or a homolog or fragment thereof, or a homolog thereof being depicted in Table I, application no. 14, column 8, in the same line as SEQ ID NO. 62079, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 62079 is depicted, plastidic is mentioned, or a mitochondrial transit peptide in case in Table I, application no. 14, in column 6 in the same line as SEQ ID NO. 62079 is depicted, mitochondric is mentioned; which is operable linked, too.

In another embodiment thereof the expression cassette does not comprise an additional transit peptide in case in Table I, application no. 14, in column 6 in the same line as SEQ ID NO. 62079 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12974, or a homolog or fragment thereof, or a homolog thereof being depicted in Table I, application no. 14, column 8, in the same line as SEQ ID NO. 12974, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 12974 is depicted, plastidic is mentioned, or a mitochondrial transit peptide in case in Table I, application no. 14, in column 6 in the same line as SEQ ID NO. 12974 is depicted, mitochondric is mentioned; which is operable linked, too.

In another embodiment thereof the expression cassette does not comprise an additional transit peptide in case in Table I, application no. 14, in column 6 in the same line as SEQ ID NO. 12974 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 113224, or a homolog or fragment thereof, or a homolog thereof being depicted in Table I, application no. 14, column 8, in the same line as SEQ ID NO. 113224, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 113224 is depicted, plastidic is mentioned, or a mitochondrial transit peptide in case in Table I, application no. 14, in column 6 in the same line as SEQ ID NO. 113224 is depicted, mitochondric is mentioned; which is operable linked, too.

In another embodiment thereof the expression cassette does not comprise an additional transit peptide in case in Table I, application no. 14, in column 6 in the same line as SEQ ID NO. 113224 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 14171, or a homolog or fragment thereof, or a homolog thereof being depicted in Table I, application no. 14, column 8, in the same line as SEQ ID NO. 14171, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 14171 is depicted, plastidic is mentioned, or a mitochondrial transit peptide in case in Table I, application no. 14, in column 6 in the same line as SEQ ID NO. 14171 is depicted, mitochondric is mentioned; which is operable linked, too.

In another embodiment thereof the expression cassette does not comprise an additional transit peptide in case in Table I, application no. 14, in column 6 in the same line as SEQ ID NO. 14171 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 113313, or a homolog or fragment thereof, or a homolog thereof being depicted in Table I, application no. 14, column 8, in the same line as SEQ ID NO. 113313, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 113313 is depicted, plastidic is mentioned, or a mitochondrial transit peptide in case in Table I, application no. 14, in column 6 in the same line as SEQ ID NO. 113313 is depicted, mitochondric is mentioned; which is operable linked, too.

In another embodiment thereof the expression cassette does not comprise an additional transit peptide in case in Table I, application no. 14, in column 6 in the same line as SEQ ID NO. 113313 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 120436, or a homolog or fragment thereof, or a homolog thereof being depicted in Table I, application no. 14, column 8, in the same line as SEQ ID NO. 120436, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 120436 is depicted, plastidic is mentioned, or a mitochondrial transit peptide in case in Table I, application no. 14, in column 6 in the same line as SEQ ID NO. 120436 is depicted, mitochondric is mentioned; which is operable linked, too.

In another embodiment thereof the expression cassette does not comprise an additional transit peptide in case in Table I, application no. 14, in column 6 in the same line as SEQ ID NO. 120436 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 66274, or a homolog or fragment thereof, or a homolog thereof being depicted in Table I, application no. 14, column 8, in the same line as SEQ ID NO. 66274, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 66274 is depicted, plastidic is mentioned, or a mitochondrial transit peptide in case in Table I, application no. 14, in column 6 in the same line as SEQ ID NO. 66274 is depicted, mitochondric is mentioned; which is operable linked, too.

In another embodiment thereof the expression cassette does not comprise an additional transit peptide in case in Table I, application no. 14, in column 6 in the same line as SEQ ID NO. 66274 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 129546, or a homolog or fragment thereof, or a homolog thereof being depicted in Table I, application no. 14, column 8, in the same line as SEQ ID NO. 129546, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 129546 is depicted, plastidic is mentioned, or a mitochondrial transit peptide in case in Table I, application no. 14, in column 6 in the same line as SEQ ID NO. 129546 is depicted, mitochondric is mentioned; which is operable linked, too.

In another embodiment thereof the expression cassette does not comprise an additional transit peptide in case in Table I, application no. 14, in column 6 in the same line as SEQ ID NO. 129546 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 92668, or a homolog or fragment thereof, or a homolog thereof being depicted in Table I, application no. 14, column 8, in the same line as SEQ ID NO. 92668, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 92668 is depicted, plastidic is mentioned, or a mitochondrial transit peptide in case in Table I, application no. 14, in column 6 in the same line as SEQ ID NO. 92668 is depicted, mitochondric is mentioned; which is operable linked, too.

In another embodiment thereof the expression cassette does not comprise an additional transit peptide in case in Table I, application no. 14, in column 6 in the same line as SEQ ID NO. 92668 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 113647, or a homolog or fragment thereof, or a homolog thereof being depicted in Table I, application no. 14, column 8, in the same line as SEQ ID NO. 113647, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 113647 is depicted, plastidic is mentioned, or a mitochondrial transit peptide in case in Table I, application no. 14, in column 6 in the same line as SEQ ID NO. 113647 is depicted, mitochondric is mentioned; which is operable linked, too.

In another embodiment thereof the expression cassette does not comprise an additional transit peptide in case in Table I, application no. 14, in column 6 in the same line as SEQ ID NO. 113647 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 16883, or a homolog or fragment thereof, or a homolog thereof being depicted in Table I, application no. 14, column 8, in the same line as SEQ ID NO. 16883, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 14, in column 6 in the same line as SEQ ID NO. 16883 is depicted, plastidic is mentioned, or a mitochondrial transit peptide in case in Table I, application no. 14, in column 6 in the same line as SEQ ID NO. 16883 is depicted, mitochondric is mentioned; which is operable linked, too.

In another embodiment thereof the expression cassette does not comprise an additional transit peptide in case in Table I, application no. 14, in column 6 in the same line as SEQ ID NO. 16883 is depicted, non-targeted is mentioned.

[0165.1.7.14] to [0170.1.7.14] for the disclosure of these paragraphs see [0165.1.7.7] to [0170.1.7.7] above

The invention further provides a vector, comprising a nucleic acid molecule comprising 1) a nucleic acid molecule selected from the group consisting of:
(a) a nucleic acid molecule encoding a polypeptide shown in Table II, column 5 or 8, application no. 14, preferably shown in Table II A, application no. 14, in column 5, or in Table II

A, application no. 14, column 8, or in Table II B, application no. 14, column 8, or a homolog or a fragment thereof;
(b) a nucleic acid molecule shown in Table I, application no. 14, column 5 or 8, preferably shown in Table I A, application no. 14, in column 5, or in Table I A, application no. 14, column 8, or in Table I B, application no. 14, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 14, preferably shown in Table II A, application no. 14, in column 5, or in Table II A, application no. 14, column 8, or in Table II B, application no. 14, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 14, preferably shown in Table I A, application no. 14, in column 5, or in Table I A, application no. 14, column 8, or in Table I B, application no. 14, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 14;
(i) a nucleic acid molecule encoding a polypeptide having the activity represented by a protein as depicted in column 5 of Table II, application no. 14;
(j) a nucleic acid molecule comprising a polynucleotide which is obtained by amplifying a cDNA library or a genomic library using the primers in Table III, application no. 14, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 14.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 14, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 14.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 14, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 14, in the same line as of the respective nucleic acid molecule according to 1) “plastidic” is depicted, or encoding a mitochondrial transit peptide, especially in case in column 6 of Table I, application no. 14, in the same line as of the respective nucleic acid molecule according to 1) “mitochondric” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 14, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 14, in the same line as of the respective nucleic acid molecule according to 1) “plastidic” is depicted, or encoding a mitochondrial transit peptide, especially in case in column 6 of Table I, application no. 14, in the same line as of the respective nucleic acid molecule according to 1) “mitochondric” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operable linked; a nucleic acid molecule encoding a transit peptide, especially in case in column 6 of Table I, application no. 14, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 14, in the same line as of the respective nucleic acid molecule according to 1) “plastidic” is depicted, or encoding a mitochondrial transit peptide, especially in case in column 6 of Table I, application no. 14, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 14 (in the same line).

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 14, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 14.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 14, in the same line as of the respective nucleic acid molecule according to 1) “plastidic” is depicted, or encoding a mitochondrial transit peptide, especially in case in column 6 of Table I, application no. 14, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 14, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 14, in the same line as of the respective nucleic acid molecule according to 1) “plastidic” is depicted, or encoding a mitochondrial transit peptide, especially in case in column 6 of Table I, application no. 14, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 14 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 14, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 14 (in the same line) and confers a generation or an increase of the respective fine chemical in a non-human organism or a part thereof as compared to a corresponding wild-type non-human organism or part thereof.

In a preferred embodiment of said vector said nucleic acid molecule according to 1 (a), (b), (c), (d), (e), (f), (g), (h), (i), (j) or (k), said promoter and optionally said nucleic acid molecule encoding a transit peptide are encompassed in an expression cassette. Accordingly in an embodiment the invention provides a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

The invention further provides a vector comprising a nucleic acid molecule comprising 1) a nucleic acid molecule selected from the group consisting of:
(a) a nucleic acid molecule encoding a polypeptide shown in Table II, column 5 or 8, application no. 14, preferably shown in Table II A, application no. 14, in column 5, or in Table II A, application no. 14, column 8, or in Table II B, application no. 14, column 8, or a homolog or a fragment thereof;
(b) a nucleic acid molecule shown in Table I, application no. 14, column 5 or 8, preferably shown in Table I A, application no. 14, in column 5, or in Table I A, application no. 14, column 8, or in Table I B, application no. 14, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 14, preferably shown in Table II A, application no. 14, in column 5, or in Table II A, application no. 14, column 8, or in Table II B, application no. 14, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 14, preferably shown in Table I A, application no. 14, in column 5, or in Table I A, application no. 14, column 8, or in Table I B, application no. 14, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 14;
(i) a nucleic acid molecule encoding a polypeptide having the activity represented by a protein as depicted in column 5 of Table II, application no. 14;
(j) a nucleic acid molecule comprising a polynucleotide which is obtained by amplifying a cDNA library or a genomic library using the primers in Table III, application no. 14, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto;
   whereby, preferably, the nucleic acid molecule according to 1 (a), (b), (c), (d), (e), (f), (g), (h), (i), (j) or (k) distinguishes over the sequence depicted in Table I A and/or I B, application no. 14, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 14, column 5 or 8, or the coding region thereof, respectively. In another embodiment, the nucleic acid molecule of the present invention, preferably the coding region thereof, is 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 14, column 5 or 8, or the coding region thereof, respectively. In a further embodiment the nucleic acid molecule does not encode the polypeptide sequence shown in Table II A and/or II B, application no. 14, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 14, column 5 or 8 and does not encode a protein of the sequence shown in Table II A and/or II B, application no. 14, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 14, column 5 and 8, or the coding region thereof. In a further embodiment, the protein of the present invention is at least 30%,40%, 50%, 60%, 70%, 80%, 90%, 95% identical to protein sequence depicted in Table II A and/or II B, application no. 14, column 5 or 8 but less than 100%, preferably less than 99.999%, 99.99% or 99.9%, more preferably less than 99%, 985, 97%, 96% or 95% identical to the sequence shown in Table II A and/or II B, application no. 14, column 5 or 8.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 14.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 14, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 14.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 14, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 14, in the same line as of the respective nucleic acid molecule according to 1) “plastidic” is depicted, or encoding a mitochondrial transit peptide, especially in case in column 6 of Table I, application no. 14, in the same line as of the respective nucleic acid molecule according to 1) “mitochondric” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 14, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 14, in the same line as of the respective nucleic acid molecule according to 1) “plastidic” is depicted, or encoding a mitochondrial transit peptide, especially in case in column 6 of Table I, application no. 14, in the same line as of the respective nucleic acid molecule according to 1) “mitochondric” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 14, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 14, in the same line as of the respective nucleic acid molecule according to 1) “plastidic” is depicted, or encoding a mitochondrial transit peptide, especially in case in column 6 of Table I, application no. 14, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 14.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 14, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 14.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 14, in the same line as of the respective nucleic acid molecule according to 1) “plastidic” is depicted, or encoding a mitochondrial transit peptide, especially in case in column 6 of Table I, application no. 14, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 14, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 14, in the same line as of the respective nucleic acid molecule according to 1) “plastidic” is depicted, or encoding a mitochondrial transit peptide, especially in case in column 6 of Table I, application no. 14, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 14 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 14, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 14 and confers a generation or an increase of the respective fine chemical in a non-human organism or a part thereof as compared to a corresponding wild-type non-human organism or part thereof.

[0181.1.7.14] to [0209.1.7.14] for the disclosure of these paragraphs see [0181.1.7.7] to [0209.1.7.7] above

The genes of the invention, coding in one embodiment for an activity selected from the group consisting of 2-oxoglutarate dehydrogenase E1 subunit, ABC metal ion transporter substrate-binding protein, acetyl-CoA acetyltransferase, acetyltransferase, acid shock protein, acyl transferase, amidophosphoribosyltransferase, ankyrin repeat family protein, ATP-binding component of a transport system, b1401-protein, B1795-protein, b2107-protein, b2376-protein, b2399-protein, b2474-protein, b2513-protein, b2654-protein, b3151-protein, b3989-protein, b4029-protein, b4205-protein, C-4 sterol methyl oxidase, calcium-dependent protein kinase, cation transport protein, cation-transporting ATPase, CTP synthase, CTP synthetase, cyclin, cyclin D, eukaryotic translation initiation factor 5, glucose dehydrogenase, glucose-6-phosphate 1-dehydrogenase, glutaredoxin, homocitrate synthase, hydrolase, lipoprotein precursor, L-ribulose-5-phosphate 4-epimerase, membrane transport protein, methyltransferase, monothiol glutaredoxin, multidrug resistance protein, multiple antibiotic resistance protein, murein transglycosylase, NADH dehydrogenase I chain N, oxidoreductase, permease protein, permease protein of ABC transporter, phenylacetic acid degradation protein, phosphoribulokinase, polygalacturonase, protein kinase, protein phosphatase, putative transport system permease protein, putrescine transport system permease protein, recombinase A, sec-independent protein translocase, sll0406-protein, sll1761-protein, sodium/proton antiporter, sterol desaturase, threonine dehydrogenase, threonine synthase, transcription factor, tryptophan synthase alpha chain, TTC1386-protein, xanthine dehydrogenase, ydl235c-protein, ynl321w-protein, and ypr170c-protein are also called “FCRP genes”.

[0211.1.7.14] to [0225.1.7.14] for the disclosure of these paragraphs see [0211.1.7.7] to [0225.1.7.7] above

In addition to the sequence mentioned in Table I, application no. 14, column 5 or 8, preferably the coding region thereof, or its homologs or fragments, it may be advantageous additionally to express and/or mutate further genes in the non-human organisms. Especially advantageously, additionally at least one further gene of the biosynthetic pathway of the fine chemical is expressed in the non-human organisms such as plants or microorganisms. It is also possible that the regulation of the natural genes has been modified advantageously so that the gene and/or its gene product is no longer subject to the regulatory mechanisms which exist in the non-human organisms. This leads to an increased synthesis of the respective amino acid since, for example, feedback regulations no longer exist to the same extent or not at all. In addition it might be advantageously to combine the nucleic acids sequences of the invention containing the sequences shown in the respective line in Table I, application no. 14, column 5 or 8, preferably the coding region thereof, or homologs or fragments thereof, with genes which generally support or enhance the growth or yield of the target non-human organisms, for example genes which lead to faster growth rate of non human organism like microorganisms or plants or genes which produce stress-, pathogen-, or herbicide-resistant plants.

In a further embodiment of the process of the invention, therefore, organisms are grown, in which there is simultaneous overexpression of at least one nucleic acid or one of the genes which code for proteins involved in the sterol/phytosterol metabolism, in particular in synthesis of of beta-sitosterol, stigmasterol and/or campesterol.

Further advantageous nucleic acid sequences which can be expressed in combination with the sequences used in the process and/or the abovementioned biosynthesis genes are the sequences encoding further genes of the phytosterol biosynthetic pathway, such as the enzymes catalyzing the production of acetyl CoA_HMGCoA, mevalonate, mevalonate 5 phosphate, mevalonate 5-pyrophosphate, isopentyl diphosphate, 5-pyrophosphatemevalonate, isopentyl pyrophosphate (PIP), dimethylallyl pyrophosphate (DMAPP), PIP+DMAPP, geranyl pyrophosphate+IPP, farnesyl pyrophosphate, 2 farnesyl pyrophosphate, squalene (squalene synthase) and squalene epoxide, or cycloartenol synthase controling the cyclization of squalene epoxide, S-adenosyl-L-methionine:sterol C-24 methyl transferase (EC 2.1.1.41) (SMT1) catalyzing the transfer of a methyl group from a cofactor, SMT2 catalyzing the second methyl transfer reaction, sterol C-14 demethylase catalyzing the demethylation at C-14, removing the methyl group and creating a double bond. These genes can lead to an increased synthesis of the essential phytosterol, in particular, of beta-sitosterol, stigmasterol and/or campesterol resp.

[0228.1.7.14] to [0239.1.7.14] for the disclosure of these paragraphs see [0228.1.7.7] to [0239.1.7.7] above

In an embodiment of the present invention the nucleic acid construct of the present invention comprises a nucleic acid molecule as depicted in the respective line in Table I, application no. 14, column 5 or 8, preferably the coding region thereof, a fragment or a homolog thereof, and a plant promoter, such as the Big 35S promoter, the DC3 promoter, the PC Ubi promoter, the LegB4-promoter, the Super promoter, the USP promoter or the like. In another embodiment the nucleic acid construct of the present invention comprises a nucleic acid molecule as depicted in the respective line in Table I, application no. 14, column 5 or 8, preferably the coding region thereof, a fragment or a homolog thereof, and a plant promoter such as, for example, the USP, the LegB4-, the DC3 promoter or the ubiquitin promoter from parsley or other herein mentioned promoters. In a further embodiment the nucleic acid construct of the present invention comprises a nucleic acid molecule as depicted in the respective line in Table I, application no. 14, column 5 or 8, preferably the coding region thereof, a fragment or a homolog thereof, and a plant promoter, such as the Big 35S promoter, the PC Ubi promoter, the Super promoter, the USP promoter or the like. In addition different terminators may advantageously be used in these nucleic acid constructs; examples for transcriptional termination are polyadenylation signals.

[0241.1.7.14] to [0245.1.7.14] for the disclosure of theses paragraphs see [0241.1.7.7] to [0245.1.7.7] above

Other preferred sequences for use in operable linkage in gene expression constructs are targeting sequences, which are required for targeting the gene product into specific cell compartments (for a review, see Kermode, Crit. Rev. Plant Sci. 15 (4), 285 (1996) and references cited therein), for example into the vacuole, the nucleus, all types of plastids, such as amyloplasts, chloroplasts, chromoplasts, the extracellular space, the mitochondria, the endoplasmic reticulum, elaioplasts, peroxisomes, glycosomes, and other compartments of cells or extracellular; preferred are sequences, which are involved in targeting to plastids as mentioned above. Sequences, which must be mentioned in this context are, in particular, the signal-peptide- or transit-peptide-encoding sequences which are known per se. For example, plastidtransit-peptide-encoding sequences enable the targeting of the expression product into the plastids of a plant cell. Targeting sequences are also known for eukaryotic and to a lower extent for prokaryotic organisms and can advantageously be operable linked with the nucleic acid molecule of the present invention as shown in Table I, application no. 14, columns 5 and 8, preferably the coding region thereof, or homologs or fragments thereof, and described herein to achieve an expression in one of said compartments or extracellular.

[0247.1.7.14] to [0249.1.7.14] for the disclosure of these paragraphs see [0247.1.7.7] to [0249.1.7.7] above

Preferred promoters are in particular those which bring about gene expression in tissues and organs, such as leaves, roots, seeds, pollen or xylem. Examples of tissue preferred or organ preferred promoters include, but are not limited to fruit-preferred, ovulepreferred, male tissue-preferred, seed-preferred, pollen-preferred integument-preferred, tuber-preferred, stalk-preferred, pericarp-preferred, leaf-preferred, stigma-preferred, pollen-preferred, anther-preferred, petal-preferred, sepal-preferred, pedicel-preferred, silique-preferred, stem-preferred, root-preferred promoters, and the like. Especially those promoters are preferred which bring about gene expression in tissues and organs in which the biosynthesis of phytosterols takes place, like in seed cells, such as endosperm cells and cells of the developing embryo. Seed promoters are preferentially expressed during seed development and/or germination. For example, seed preferred promoters can be embryo-preferred, endosperm preferred and seed coat-preferred (see Thompson et al., BioEssays 10, 108 (1989)). Examples of seed preferred promoters include, but are not limited to, cellulose synthase (celA), Cim1, gammazein, globulin-1, maize 19 kD zein (cZ19B1), and the like. Other suitable promoters are the oilseed rape napin gene promoter (U.S. Pat. No. 5,608,152), the Vicia faba USP promoter (Baeumlein et al., Mol Gen Genet, 225 (3), 459 (1991)), the Arabidopsis oleosin promoter (WO 98/45461), the Phaseolus vulgaris phaseolin promoter (U.S. Pat. No. 5,504,200), the Brassica Bce4 promoter (WO 91/13980), the bean arc5 promoter, the carrot DcG3 promoter, or the Legumin B4 promoter (LeB4) (Baeumlein et al., Plant Journal, 2 (2), 233 (1992)), and promoters which bring about the seed-specific expression in monocotyledonous plants such as maize, barley, wheat, rye, rice and the like. Advantageous seed-specific promoters are the sucrose binding protein promoter (WO 00/26388), the phaseolin promoter and the napin promoter. Suitable promoters which must be considered are the barley Ipt2 or Ipt1 gene promoter (WO 95/15389 and WO 95/23230), and the promoters described in WO 99/16890 (promoters from the barley hordein gene, the rice glutelin gene, the rice oryzin gene, the rice prolamin gene, the wheat gliadin gene, the wheat glutelin gene, the maize zein gene, the oat glutelin gene, the sorghum kasirin gene and the rye secalin gene). Further suitable promoters are Amy32b, Amy 6-6 and Aleurain (U.S. Pat. No. 5,677,474), Bce4 (oilseed rape) (U.S. Pat. No. 5,530,149), glycinin (soya) (EP 571 741], phosphoenolpyruvate carboxylase (soya) (JP 06/62870), ADR12-2 (soya) (WO 98/08962), isocitrate lyase (oilseed rape) (U.S. Pat. No. 5,689,040]) or α-amylase (barley) (EP 781 8499. Other promoters which are available for the expression of genes in plants are leaf-specific promoters such as those described in DE-A 19 644 478 or light-regulated promoters such as, for example, the pea petE promoter.

[0251.1.7.14] to [0266.1.7.14] for the disclosure of these paragraphs see [0251.1.7.7] to [0266.1.7.7] above

An embodiment of the invention relates to a vector where the nucleic acid molecule according to the invention is linked operably to regulatory sequences which permit the expression in a prokaryotic or eukaryotic host. A further embodiment of the invention relates to a vector in which a nucleic acid sequence encoding one of the polypeptides shown in the respective line in Table II, application no. 14, columns 5 or 8, or homologs or fragments thereof, is functionally linked to a targeting sequence such as a plastidial or mitochondrial targeting sequence. A further embodiment of the invention relates to a vector in which a nucleic acid sequence encoding one of the polypeptides shown in the respective line in Table II, application no. 14, columns 5 or 8, or homologs or fragments thereof, is functionally linked to (a) regulatory sequence which permit the expression in an organelle, such as plastids or mitochondria, preferably plastids.

A further embodiment of the invention relates to a vector which comprises a nucleic acid sequence encoding one of the polypeptides shown in the respective line in Table II, application no. 14, columns 5 or 8, or homologs or fragments thereof, but no additional targeting sequence.

[0268.1.7.14] to [0273.1.7.14] for the disclosure of these paragraphs see [0268.1.7.7] to [0273.1.7.7] above

A further object of the invention relates to the use of a nucleic acid construct, e.g. an expression cassette, comprising one or more nucleic acid molecules encoding one or more polypeptides as depicted in the respective line(s) in Table II, application no. 14, columns 5 or 8, or homologs or fragments thereof, or comprising one or more nucleic acid molecules as depicted in the respective line(s) in Table I, application no. 14, columns 5 or 8, preferably the coding region thereof, or homologs or fragments thereof, or comprising a nucleic acid molecule hybridizing therewith for the transformation of a microorganism or a plant cell, plant tissues or a part of a plant

In doing so, depending on the choice of promoter, the nucleic acid molecules shown in the respective line in Table I, application no. 14, column 5 or 8, preferably the coding region thereof, or homologs or fragments thereof, or the nucleic acid molecules encoding a polypeptide as depicted in the respective line in Table II, application no. 14, column 5 or 8, or homologs or fragments thereof, can be expressed in the whole plant or specifically e.g. in the leaves, in the seeds, the nodules, in roots, in the stem or other parts of the plant. Those transgenic plants overproducing sequences, e.g. as depicted in the respective line in Table I, application no. 14, column 5 or 8, preferably the coding region thereof, or homologs or fragments thereof, the plant cell, plant tissues, plants, the reproductive material thereof, or parts thereof are a further object of the present invention.

Within the framework of the present invention, the production of the beta-sitosterol, campesterol, or stigmasterol is due to the generation or over-expression of one or more polypeptides as depicted in the respective line(s) in Table II, application no. 14, column 5 or 8, or homologs or fragments thereof, or encoded by the corresponding nucleic acid molecules as depicted in the respective line(s) in Table I, application no. 14, column 5 or 8, preferably the coding region thereof, or homologs or fragments thereof, in the non-human organism according to the invention, advantageously in the transgenic microorganism or plant according to the invention, at least for the duration of at least one plant generation.

A constitutive expression of the polypeptide as depicted in the respective line in Table II, application no. 14, column 5 or 8, or homologs or fragments thereof, or encoded by the respective nucleic acid molecule as depicted in the respective line in Table I, application no. 14, column 5 or 8, preferably the coding region thereof, or homologs or fragments thereof, may be advantageous. On the other hand, an inducible expression may also appear desirable. Expression of the polypeptide sequences of the invention can be either directed to the cytosol or to the organelles, such as plastids or mitochondria, preferably the plastids of the host cells, preferably the plant cells, or non-targeted.

for the disclosure of this paragraph see [0278.1.7.7] above

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 14, column 5 or 8, preferably shown in Table II A, application no. 14, column 5, or in Table II A, application no. 14, column 8, or in Table II B, application no. 14, column 8, or a homolog or a fragment thereof;
   (b) a nucleic acid molecule shown in Table I, application no. 14, column 5 or 8, preferably shown in Table I A, application no. 14, column 5, or in Table I A, application no. 14, column 8, or in Table I B, application no. 14, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 14, preferably shown in Table II A, application no. 14, column 5, or in Table II A, application no. 14, column 8, or in Table II B, application no. 14, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 14, preferably shown in Table I A, application no. 14, column 5, or in Table I A, application no. 14, column 8, or in Table I B, application no. 14, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 14;
   (i) a nucleic acid molecule encoding a polypeptide having the activity represented by a respective protein as depicted in column 5 of Table II, application no. 14;
   (j) a nucleic acid molecule comprising a polynucleotide which is obtained by amplifying a cDNA library or a genomic library using the primers in Table III, application no. 14, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto; and, if desired
2) nucleic acid molecule encoding a transit peptide, preferably encoding a plastidal transit peptide or a mitochondrial transit peptide; which are operable linked;]
   or comprising an expression cassette comprising
[0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 14, column 5 or 8, preferably shown in Table II A, application no. 14, column 5, or in Table II A, application no. 14, column 8, or in Table II B, application no. 14, column 8, or a homolog or a fragment thereof;
   (b) a nucleic acid molecule shown in Table I, application no. 14, column 5 or 8, preferably shown in Table I A, application no. 14, column 5, or in Table I A, application no. 14, column 8, or in Table I B, application no. 14, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 14, preferably shown in Table II A, application no. 14, column 5, or in Table II A, application no. 14, column 8, or in Table II B, application no. 14, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 14, preferably shown in Table I A, application no. 14, column 5, or in Table I A, application no. 14, column 8, or in Table I B, application no. 14, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 14;
   (i) a nucleic acid molecule encoding a polypeptide having the activity represented by a respective protein as depicted in column 5 of Table II, application no. 14;
   (j) a nucleic acid molecule comprising a polynucleotide which is obtained by amplifying a cDNA library or a genomic library using the primers in Table III, application no. 14, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising or being transformed by a vector comprising
[1) a nucleic acid molecule selected from the group consisting of
   (a) a nucleic acid molecule encoding a polypeptide shown in Table II, application no.

14, column 5 or 8, preferably shown in Table II A, application no. 14, column 5, or in Table II A, application no. 14, column 8, or in Table II B, application no. 14, column 8, or a homolog or a fragment thereof;
(b) a nucleic acid molecule shown in Table I, application no. 14, column 5 or 8, preferably shown in Table I A, application no. 14, column 5, or in Table I A, application no. 14, column 8, or in Table I B, application no. 14, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 14, preferably shown in Table II A, application no. 14, column 5, or in Table II A, application no. 14, column 8, or in Table II B, application no. 14, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 14, preferably shown in Table I A, application no. 14, column 5, or in Table I A, application no. 14, column 8, or in Table I B, application no. 14, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 14;
(i) a nucleic acid molecule encoding a polypeptide having the activity represented by a respective protein as depicted in column 5 of Table II, application no. 14;
(j) a nucleic acid molecule comprising a polynucleotide which is obtained by amplifying a cDNA library or a genomic library using the primers in Table III, application no. 14, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
or a nucleic acid molecule comprising a sequence which is complementary thereto;]
   as well as respective transgenic cells, tissue, parts of such non-human organism, e.g. plant cells, plant tissue, part of plants, like leaves, roots, stems, blossoms, seeds, fruits, pollen and the like.
   (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 14.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 14 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 14, in the same line as of the respective nucleic acid molecule according to 1) “plastidic” is depicted, or encoding a mitochondrial transit peptide, especially in case in column 6 of Table I, application no. 14, in the same line as of the respective nucleic acid molecule according to 1) “mitochondric” is depicted.

In another embodiment of said transgenic non-human organism the expression cassette does not comprise 2) a nucleic acid molecule encoding a transit peptide, especially in case in column 6 of Table I, application no. 14, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 14.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 14, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 14, in the same line as of the respective nucleic acid molecule according to 1) “plastidic” is depicted, or encoding a mitochondrial transit peptide, especially in case in column 6 of Table I, application no. 14, in the same line as of the respective nucleic acid molecule according to 1) “mitochondric” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 14, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 14, in the same line as of the respective nucleic acid molecule according to 1) “plastidic” is depicted, or encoding a mitochondrial transit peptide, especially in case in column 6 of Table I, application no. 14, in the same line as of the respective nucleic acid molecule according to 1) “mitochondric”. is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 14, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 14, in the same line as of the respective nucleic acid molecule according to 1) “plastidic” is depicted, or encoding a mitochondrial transit peptide, especially in case in column 6 of Table I, application no. 14, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 14.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 14, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 14.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 14, in the same line as of the respective nucleic acid molecule according to 1) “plastidic” is depicted, or encoding a mitochondrial transit peptide, especially in case in column 6 of Table I, application no. 14, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 14, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 14, in the same line as of the respective nucleic acid molecule according to 1) “plastidic” is depicted, or encoding a mitochondrial transit peptide, especially in case in column 6 of Table I, application no. 14, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 14 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 14, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 14 (in the same line) and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 14, column 5 or 8, preferably shown in Table II A, application no. 14, column 5, or in Table II A, application no. 14, column 8, or in Table II B, application no. 14, column 8, or a homolog or a fragment thereof;
   (b) a nucleic acid molecule shown in Table I, application no. 14, column 5 or 8, preferably shown in Table I A, application no. 14, column 5, or in Table I A, application no. 14, column 8, or in Table I B, application no. 14, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 14, preferably shown in Table II A, application no. 14, column 5, or in Table II A, application no. 14, column 8, or in Table II B, application no. 14, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 14, preferably shown in Table I A, application no. 14, column 5, or in Table I A, application no. 14, column 8, or in Table I B, application no. 14, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 14;
   (i) a nucleic acid molecule encoding a polypeptide having the activity represented by a respective protein as depicted in column 5 of Table II, application no. 14;
   (j) a nucleic acid molecule comprising a polynucleotide which is obtained by amplifying a cDNA library or a genomic library using the primers in Table III, application no.

14, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
or a nucleic acid molecule comprising a sequence which is complementary thereto;
   and, if desired
2) nucleic acid molecule encoding a transit peptide, preferably encoding a plastidal transit peptide or a mitochondrial transit peptide;
   which are operable linked;]
   or comprising an expression cassette comprising
[0) a promoter, preferably a promoter selected from the group consisting of Big35S, PCUbi, Super and USP; and 1) a nucleic acid molecule selected from the group consisting of
   (a) a nucleic acid molecule encoding a polypeptide shown in Table II, application no. 14, column 5 or 8, preferably shown in Table II A, application no. 14, column 5, or in Table II A, application no. 14, column 8, or in Table II B, application no. 14, column 8, or a homolog or a fragment thereof;
   (b) a nucleic acid molecule shown in Table I, application no. 14, column 5 or 8, preferably shown in Table I A, application no. 14, column 5, or in Table I A, application no. 14, column 8, or in Table I B, application no. 14, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 14, preferably shown in Table II A, application no. 14, column 5, or in Table II A, application no. 14, column 8, or in Table II B, application no. 14, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 14, preferably shown in Table I A, application no. 14, column 5, or in Table I A, application no. 14, column 8, or in Table I B, application no. 14, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 14;
   (i) a nucleic acid molecule encoding a polypeptide having the activity represented by a respective protein as depicted in column 5 of Table II, application no. 14;
   (j) a nucleic acid molecule comprising a polynucleotide which is obtained by amplifying a cDNA library or a genomic library using the primers in Table III, application no. 14, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      and, if desired
2) nucleic acid molecule encoding a transit peptide, preferably encoding a plastidal transit peptide or a mitochondrial transit peptide;
   which are operable linked;]
   or comprising or being transformed by a vector comprising
[1) a nucleic acid molecule selected from the group consisting of
   (a) a nucleic acid molecule encoding a polypeptide shown in Table II, application no. 14, column 5 or 8, preferably shown in Table II A, application no. 14, column 5, or in Table II A, application no. 14, column 8, or in Table II B, application no. 14, column 8, or a homolog or a fragment thereof;
   (b) a nucleic acid molecule shown in Table I, application no. 14, column 5 or 8, preferably shown in Table I A, application no. 14, column 5, or in Table I A, application no. 14, column 8, or in Table I B, application no. 14, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 14, preferably shown in Table II A, application no. 14, column 5, or in Table II A, application no. 14, column 8, or in Table II B, application no. 14, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 14, preferably shown in Table I A, application no. 14, column 5, or in Table I A, application no. 14, column 8, or in Table I B, application no. 14, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 14;
   (i) a nucleic acid molecule encoding a polypeptide having the activity represented by a respective protein as depicted in column 5 of Table II, application no. 14;
   (j) a nucleic acid molecule comprising a polynucleotide which is obtained by amplifying a cDNA library or a genomic library using the primers in Table III, application no. 14, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      whereby, preferably, the nucleic acid molecule according to 1 (a), (b), (c), (d), (e), (f), (g), (h), (i), (j) or (k) distinguishes over the sequence depicted in Table I A and/or I B, application no. 14, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 14, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 14, column 5 or 8, or the coding region thereof, respectively. In a further embodiment the nucleic acid molecule does not encode the polypeptide sequence shown in Table II A and/or II B, application no. 14, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 14, column 5 or 8 and does not encode a protein of the sequence shown in Table II A and/or II B, application no. 14, column 5 or 8. Accordingly, in one embodiment, the protein encoded by a sequence of a nucleic acid according to 1 (a), (b), (c), (d), (e), (f), (g), (h), (i), (j) or (k) does not consist of the sequence shown in Table I A and/or I B, application no. 14, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 14, column 5 or 8 but less than 100%, preferably less than 99.999%, 99.99%, 99.9%, 99%, 98%, 97%, 96% or 95% identical to the sequence shown in Table II A and/or II B, application no. 14, column 5 or 8; as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 14.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 14 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 14, in the same line as of the respective nucleic acid molecule according to 1) “plastidic” is depicted, or encoding a mitochondrial transit peptide, especially in case in column 6 of Table I, application no. 14, in the same line as of the respective nucleic acid molecule according to 1) “mitochondric” is depicted.

In another embodiment thereof the expression cassette does not comprise 2) a nucleic acid molecule encoding a transit peptide, especially in case in column 6 of Table I, application no. 14, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 14.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the same line in column 5 of Table II, application no. 14, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 14, in the same line as of the respective nucleic acid molecule according to 1) “plastidic” is depicted, or encoding a mitochondrial transit peptide, especially in case in column 6 of Table I, application no. 14, in the same line as of the respective nucleic acid molecule according to 1) “mitochondric” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 14, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 14, in the same line as of the respective nucleic acid molecule according to 1) “plastidic” is depicted, or encoding a mitochondrial transit peptide, especially in case in column 6 of Table I, application no. 14, in the same line as of the respective nucleic acid molecule according to 1) “mitochondric” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 14, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 14, in the same line as of the respective nucleic acid molecule according to 1) “plastidic” is depicted, or encoding a mitochondrial transit peptide, especially in case in column 6 of Table I, application no. 14, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 14.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 14, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 14.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 14, in the same line as of the respective nucleic acid molecule according to 1) “plastidic” is depicted, or encoding a mitochondrial transit peptide, especially in case in column 6 of Table I, application no. 14, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 14, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 14, in the same line as of the respective nucleic acid molecule according to 1) “plastidic” is depicted, or encoding a mitochondrial transit peptide, especially in case in column 6 of Table I, application no. 14, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 14 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 14, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 14 and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

[0291.1.7.14] to [0299.1.7.14] for the disclosure of these paragraphs see [0291.1.7.7] to [0299.1.7.7] above

The term “biological active portion” or “biological activity” means a polypeptide as depicted in the respective line in Table II, application no. 14, column 3 or a portion of said polypeptide which still has at least 10% or 20%, preferably 30%, 40%, 50% or 60%, especially preferably 70%, 75%, 80%, 90% or 95% of the enzymatic or biological activity of the natural or starting enzyme or protein.

[0301.1.7.14] to [0304.1.7.14] for the disclosure of these paragraphs see [0301.1.7.7] to [0304.1.7.7] above

Synthetic oligonucleotide primers for the amplification, e.g. as shown in the respective line in Table III, application no. 14, column 8, by means of polymerase chain reaction can be generated on the basis of a sequence shown herein, for example the sequence shown in the respective line in Table I, application no. 14, columns 5 or 8, or the sequences derived from Table II, application no. 14, columns 5 or 8, respectively.

Moreover, it is possible to identify a conserved region of a protein from various organism by carrying out protein sequence alignments with the polypeptide encoded by the nucleic acid molecules of the present invention, in particular with the sequences encoded by the nucleic acid molecule shown in the respective line in column 5 or 8 of Table I, application no. 14, from which conserved regions, and in turn, degenerate primers can be derived. Conserved regions are those, which show a very little variation in the amino acid in one particular position of several homologs from different origin. The consensus sequence and polypeptide motifs shown in the respective line in column 8 of Table IV, application no. 14, are derived from such alignments of homologous proteins.

In an embodiment of the present invention, in the method of the present invention the activity of a polypeptide comprising or consisting of a consensus sequence or at least one polypeptide motif shown in the respective line in Table IV, application no. 14, column 8, is increased. In another embodiment thereof, the method of the present invention relates to a polypeptide comprising or consisting of a consensus sequence shown in the respective line in Table IV, application no. 14, column 8 or comprising at least one polypeptide motif shown in the respective line in Table IV, application no. 14, column 8, whereby not more than 20, 15, 10, 9, 8, 7, 6, 5 4, 3, 2 or 1, or 0 of the amino acid positions indicated can be replaced by any amino acid. In another embodiment thereof, the method of the present invention relates to a polypeptide comprising or consisting of a consensus sequence shown in the respective line in Table IV, application no. 14, column 8 or comprising at least one polypeptide motif shown in the respective line in Table IV, application no. 14, column 8, whereby not more than 15%, 10%, 5%, 4%, 3%, 2% or 1%, or 0% of the amino acid positions defined as distinct specific amino acids are/is replaced by another amino acid. In another embodiment thereof, the method of the present invention relates to a polypeptide comprising or consisting of a consensus sequence shown in the respective line in Table IV, application no. 14, column 8 or comprising at least one polypeptide motif shown in the respective line in Table IV, application no. 14, column 8, whereby not more than 20, 15, 10, 9, 8, 7, 6, 5, 4, 3, 2 or 1, or 0 amino acids are inserted into a consensus sequence or polypeptide motif.

In an embodiment of the present invention, in the method of the present invention the activity of a polypeptide comprising or consisting of a consensus sequence or at least one polypeptide motif shown in the respective line in Table IV, application no. 14, column 8, is increased, whereby said polypeptide distinguishes over the sequence depicted in Table II, application no. 14, columns 5 or 8 by one or more amino acids.

In one embodiment, said polypeptide of the invention distinguishes over the sequence shown in the respective line in Table II, application no. 14, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II B, application no. 14, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in Table II, application no. 14, columns 5 or 8.

[0309.1.7.14] to [0321.1.7.14] for the disclosure of these paragraphs see [0309.1.7.7] to [0321.1.7.7] above

Polypeptides having above-mentioned activity, i.e. conferring the production or the increased production of the fine chemical beta-sitosterol, campesterol, or stigmasterol as compared to a corresponding, e.g. non-transformed, wild type non-human organism, like a microorganism or a plant cell, plant or part thereof, can be encoded by other DNA sequences which hybridize to the sequences shown in the respective line in Table I, application no. 14, columns 5 and 8, preferably the coding region thereof, at least under relaxed hybridization conditions and which encode the expression of polypeptides conferring the production or the increased production of the respective fine chemical beta-sitosterol, campesterol, or stigmasterol as compared to a corresponding, e.g. non-transformed, wild type non-human organism, like a microorganism or a plant cell, plant or part thereof.

[0323.1.7.14] to [0329.1.7.14] for the disclosure of theses paragraphs see [0323.1.7.7] to [0329.1.7.7] above

Further, the nucleic acid molecule of the invention comprises a nucleic acid molecule, which is a complement of one of the nucleotide sequences of above-mentioned nucleic acid molecules or a portion thereof. A nucleic acid molecule or its sequence which is complementary to one of the nucleotide molecules or sequences shown in the respective line in Table I, application no. 14, columns 5 or 8, preferably the coding region thereof, homologs or fragments thereof, is one which is sufficiently complementary to one of the nucleotide molecules or sequences shown in the respective line in Table I, application no. 14, columns 5 or 8, preferably the coding region thereof, homologs or fragments thereof, such that it can hybridize to one of the nucleotide sequences shown in Table I, application no. 14, columns 5 or 8, preferably the coding region thereof, homologs or fragments thereof, thereby forming a stable duplex. Preferably, the hybridization is performed under stringent hybridization conditions. However, a complement of one of the herein disclosed sequences is preferably a sequence complement thereto according to the base pairing of nucleic acid molecules well known to the skilled person. For example, the bases A and G undergo base pairing with the bases T and U or C, resp. and visa versa. Modifications of the bases can influence the base-pairing partner.

The nucleic acid molecule of the invention comprises a nucleotide sequence which is at least about 30%, 35%, 40%, 45%, 50%, 55%, 60%, 65%, 70%, 75%, 80%, 90%, 95%, 96%, 97%, 98%, 99%, 99.5% or more homologous to a nucleotide sequence shown in Table I, application no. 14, columns 5 or 8, preferably the coding region thereof, homologs or fragments thereof, and preferably has above-mentioned activity, in particular conferring the production or the increased production of the fine chemical beta-sitosterol, campesterol, or stigmasterol, respectively, after increasing the activity or an activity of a gene as shown in the respective line in Table I or of a gene product, e.g. as shown in the respective line in Table II, application no. 14, column 5 or 8, by for example in one embodiment expression either in the cytosol or in an organelle such as a plastid or mitochondria or both, preferably in a plastid, or in another embodiment non-targeted or targeted.

The nucleic acid molecule of the invention comprises a nucleotide sequence or molecule which hybridizes, preferably hybridizes under stringent conditions as defined herein, to one of the nucleic acid molecule shown in the respective line in Table I, preferably Table IB, application no. 14, columns 5 or 8, preferably the coding region thereof, fragments or homologs thereof, and encodes a protein having above-mentioned activity, e.g. conferring the production or the increased production of the respective fine chemical beta-sitosterol, campesterol, or stigmasterol as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted, and optionally, the activity thereof is selected from the group consisting of 2-oxoglutarate dehydrogenase E1 subunit, ABC metal ion transporter substrate-binding protein, acetyl-CoA acetyltransferase, acetyltransferase, acid shock protein, acyl transferase, amidophosphoribosyltransferase, ankyrin repeat family protein, ATP-binding component of a transport system, b1401-protein, B1795-protein, b2107-protein, b2376-protein, b2399-protein, b2474-protein, b2513-protein, b2654-protein, b3151-protein, b3989-protein, b4029-protein, b4205-protein, C-4 sterol methyl oxidase, calcium-dependent protein kinase, cation transport protein, cation-transporting ATPase, CTP synthase, CTP synthetase, cyclin, cyclin D, eukaryotic translation initiation factor 5, glucose dehydrogenase, glucose-6-phosphate 1-dehydrogenase, glutaredoxin, homocitrate synthase, hydrolase, lipoprotein precursor, L-ribulose-5-phosphate 4-epimerase, membrane transport protein, methyltransferase, monothiol glutaredoxin, multidrug resistance protein, multiple antibiotic resistance protein, murein transglycosylase, NADH dehydrogenase I chain N, oxidoreductase, permease protein, permease protein of ABC transporter, phenylacetic acid degradation protein, phosphoribulokinase, polygalacturonase, protein kinase, protein phosphatase, putative transport system permease protein, putrescine transport system permease protein, recombinase A, sec-independent protein translocase, sll0406-protein, sll1761-protein, sodium/proton antiporter, sterol desaturase, threonine dehydrogenase, threonine synthase, transcription factor, tryptophan synthase alpha chain, TTC1386-protein, xanthine dehydrogenase, ydl235c-protein, ynl321w-protein, and ypr170c-protein, respectively.

Moreover, the nucleic acid molecule of the invention can comprise only a porganism tion of the coding region of one of the nucleic acid molecules depicted in Table I, application no. 14, columns 5 or 8, for example a fragment which can be used as a probe or primer or a fragment encoding a biologically active portion of the polypeptide of the present invention or of a polypeptide used in the process of the present invention, i.e. having above-mentioned activity, e.g. conferring the production or the increased production of the respective fine chemical beta-sitosterol, campesterol, or stigmasterol as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted. The nucleotide sequences determined from the cloning of the present protein-accordingto-the-invention-encoding gene allows for the generation of probes and primers designed for use in identifying and/or cloning its homologues from other cell types and organisms. The probe/primer typically comprises a substantially purified oligonucleotide. The oligonucleotide typically comprises a region of nucleotide sequence that hybridizes under stringent conditions to at least about 12, 15 preferably about 20 or 25, more preferably about 40, 50 or 75 consecutive nucleotides of a sense strand of one of the sequences set forth, e.g., in the respective line in Table I, application no. 14, column 5 or 8, preferably the coding region thereof, or a homolog or a fragment thereof, an anti-sense sequence of one of the sequences, e.g., set forth in the respective line in Table I, application no. 14, columns 5 or 8, preferably the coding region thereof, or a homolog or a fragment thereof or naturally occurring mutants thereof. Primers based on a nucleic acid molecule of invention can be used in PCR reactions to clone homologs of the polypeptide of the invention or of the polypeptide used in the process of the invention, e.g. as the primers described in the examples of the present invention, e.g. as shown in the examples. A PCR with the primers shown in the respective line in Table III, column 8 will result in a fragment of the gene product as shown in Table II, application no. 14, column 5.

Primer sets are interchangeable. The person skilled in the art knows how to design and combine said primers to result in the desired product, e.g. in a full length clone or a partial sequence. Probes based on the sequences of the nucleic acid molecule of the invention or used in the process of the present invention can be used to detect transcripts or genomic sequences encoding the same or homologous proteins. The probe can further comprise a label group attached thereto, e.g. the label group can be a radioisotope, a fluorescent compound, an enzyme, or an enzyme co-factor. Such probes can be used as a part of a genomic marker test kit for identifying cells which express a polypeptide of the invention or used in the process of the present invention, such as by measuring a level of an encoding nucleic acid molecule in a sample of cells, e.g. detecting mRNA levels or determining, whether a genomic gene comprising the sequence of the polynucleotide of the invention or used in the processes of the present invention has been mutated or deleted.

As the expression of a respective nucleic acids of the invention is related to the synthesis of the respective fine chemical beta-sitosterol, campesterol, or stigmasterol its function as a probe extends to the detection of microorganisms, plant tissues, plants, plant variets, plant ecotypes or plant genera with varying capability or potential for synthesis of the respective fine chemical beta-sitosterol, campesterol, or stigmasterol. Therefore in one embodiment the present invention relates to a method for analyzing the capability or potential of a plant tissue, a plant, a plant variety or plant ecotype to produce the fine chemical beta-sitosterol, campesterol, or stigmasterol by using the nucleic acid of the invention or parts thereof as a probe to detect the amount of the nucleic acid of the invention in the non-human organism or a part thereof in comparison to another non-human organism.

The nucleic acid molecule of the invention encodes a polypeptide or portion thereof which includes an amino acid sequence which is sufficiently homologous to the amino acid sequence shown in respective line in Table II, application no. 14, columns 5 or 8 such that the protein or portion thereof maintains the ability to confer the production or the increased production of the respective fine chemical beta-sitosterol, campesterol, or stigmasterol as compared to a corresponding, e.g. non-transformed, wild type non-human organism, like a microorganism or a plant cell, plant or part thereof, in particular increasing the activity as mentioned above or as described in the examples in microorganisms or plants.

As used herein, the language “sufficiently homologous” refers to proteins or portions thereof which have amino acid sequences which include a minimum number of identical or equivalent amino acids (e.g. an amino acid which has a similar side chain as an amino acid in one of the sequences of the polypeptide of the present invention) to an amino acid sequence shown in the respective line in Table II, application no. 14, columns 5 or 8 such that the protein or portion thereof is able to confer the production or the increased production of the respective fine chemical beta-sitosterol, campesterol, or stigmasterol as compared to a corresponding, e.g. non-transformed, wild type non-human organism, like a microorganism or a plant cell, plant or part thereof. For example having the activity of a protein as shown in the respective line in Table II, application no. 14, column 3 and as described herein.

In an embodiment of the present invention, the nucleic acid molecule of the present invention comprises a nucleic acid molecule that encodes a portion of the protein of the present invention. The protein is at least about 30%, 35%, 40%, 45% 50%, 55%, 60%, 65%, 70%, 75%, 80%, 85%, 90%, 91%, 92%, 93%, 94%, 95%, 96%, 97%, 98%, 99%, 99.5% or more homologous to an entire amino acid sequence shown in the respective line of Table II, application no. 14, column 5 or 8 and has the above-mentioned activity, e.g. conferring the production or the increased production of the respective fine chemical beta-sitosterol, campesterol, or stigmasterol as compared to a corresponding, e.g. non-transformed, wild type non-human organism, like a microorganism or a plant cell, plant or part thereof by, for example in a embodiment expression either in the cytosol or in an organelle such as a plastid or mitochondria or both, preferably in a plastid, or in another embodiment by targeted or non-targeted expression.

for the disclosure of this paragraph see [0338.1.7.7] above

for the disclosure of this paragraph see [0339.1.7.7] above

The invention further relates to nucleic acid molecules or methods using said nucleic acid molecules that differ from one of the nucleotide sequences shown in the respective line in Table I, application no. 14, columns 5 or 8, preferably the coding region thereof, or fragments or homologs thereof, due to degeneracy of the genetic code but encode a polypeptide of the present invention, in particular a polypeptide having the above-mentioned activity, e.g. as those polypeptides depicted in the respective line in Table II, application no. 14, columns 5 or 8, fragments or homologs thereof. Advantageously, the nucleic acid molecule of the invention comprises, or in an other embodiment has, a nucleotide sequence encoding a protein comprising, or in an other embodiment having, an amino acid sequence shown in the respective line in Table II, application no. 14, columns 5 or 8, fragments or homologs thereof. In a further embodiment, the nucleic acid molecule of the invention encodes a full length protein which is substantially homologous to an amino acid sequence shown in the respective line in Table II, application no. 14, columns 5 or 8, or homologs thereof. However, in an embodiment, the nucleic acid molecule of the present invention does not consist of the sequence shown in the respective line in Table I, preferably Table IA, application no. 14, columns 5 or 8, preferably the coding region thereof, but the functional homologs thereof.

[0341.1.7.14] to [0343.1.7.14] for the disclosure of these paragraphs see [0341.1.7.7] to [0343.1.7.7] above

Accordingly, in another embodiment, a nucleic acid molecule of the invention is at least 15, 20, 25 or 30 nucleotides in length. Preferably, it hybridizes under stringent conditions to a nucleic acid molecule comprising a nucleotide sequence of the nucleic acid molecule of the present invention or used in the process of the present invention, e.g. comprising the sequence shown in the respective line in Table I, application no. 14, columns 5 or 8, preferably the coding region thereof. In another embodiment the nucleic acid molecule is preferably at least 20, 30, 50, 100, 250 or more nucleotides in length.

for the disclosure of this paragraph see [0345.1.7.7] above

Preferably, a nucleic acid molecule of the invention that hybridizes under stringent conditions to a sequence shown in the respective line in Table I, application no. 14, columns 5 or 8, preferably the coding region thereof, corresponds to a naturally-occurring nuoleic acid molecule of the invention. As used herein, a “naturally-occurring” nucleic acid molecule refers to a RNA or DNA molecule having a nucleotide sequence that occurs in nature (e.g. encodes a natural protein). Preferably, the nucleic acid molecule encodes a natural protein having above-mentioned activity, e.g. conferring the production or the increased production of the respective fine chemical beta-sitosterol, campesterol, or stigmasterol as compared to a corresponding, e.g. non-transformed, wild type non-human organism, like a microorganism or a plant cell, plant or part thereof after increasing the expression or activity thereof or the activity of a protein of the invention or used in the process of the invention, in an embodiment for example expression either in the cytosol or in an organelle such as a plastid or mitochondria, preferably in plastids, or, in another embodiment by targeted or non-targeted expression.

for the disclosure of this paragraph see [0347.1.7.7] above

For example, nucleotide substitutions leading to amino acid substitutions at “non-essential” amino acid residues can be made in a sequence of the nucleic acid molecule of the invention or used in the process of the invention, e.g. shown in the respective line in Table I, application no. 14, columns 5 or 8, preferably the coding region thereof, or fragments or homologs thereof.

for the disclosure of this paragraph see [0349.1.7.7] above

for the disclosure of this paragraph see [0350.1.7.7] above

Accordingly, the invention relates to nucleic acid molecules encoding a polypeptide having above-mentioned activity, in a non-human organisms or parts thereof by for example expression either in the cytosol or in an organelle such as a plastid or mitochondria or both, preferably in plastids, that contain changes in amino acid residues that are not essential for said activity. Such polypeptides differ in amino acid sequence from a sequence depicted in the respective line in Table II, especially Table IIA, application no. 14, columns 5 or 8, but retain said activity described herein. The nucleic acid molecule can comprise a nucleotide sequence encoding a polypeptide, wherein the polypeptide comprises an amino acid sequence at least about 50% identical to an amino acid sequence shown in the respective line in Table II, application no. 14, columns 5 or 8, and is capable of conferring the production or the increased production of the respective fine chemical beta-sitosterol, campesterol, or stigmasterol as compared to a corresponding, e.g. non-transformed, wild type non-human organism, like a microorganism or a plant cell, plant or part thereof, after increasing its activity for example in an embodiment by expression either in the cytosol or in an organelle such as a plastid or mitochondria or both, preferably in plastids, or, in another embodiment by targeted or non-targeted expression. Preferably, the protein encoded by the nucleic acid molecule is at least about 60%, 70%, 80%, 90%, 95%, 96%, 97%, 98%, 99% or 99.5% identical to the sequence shown in the respective line in Table II, application no. 14, columns 5 or 8.

[0352.1.7.14] to [0357.1.7.14] for the disclosure of these paragraphs see [0352.1.7.7] to [0357.1.7.7] above

Functional equivalents of the nucleic acid molecules according to the present invention and/or used in the process according to the present invention represent an embodiment of homologs. Functional equivalents derived from the nucleic acid molecules as shown in the respective line in Table I, application no. 14, columns 5 or 8, preferably the coding region thereof, according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the nucleic acid molecules as shown in the respective line in Table I, application no. 14, columns 5 or 8, preferably the coding region thereof, according to the invention and encode polypeptides having essentially the same properties as the polypeptide as shown in the respective line in Table II, application no. 14, columns 5 or 8.

Functional equivalents derived from one of the polypeptides as shown in the respective line in Table II, application no. 14, columns 5 or 8 according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the polypeptides as shown in the respective line in Table II, application no. 14, columns 5 or 8 according to the invention and having essentially the same properties as the polypeptide as shown in the respective line in Table II, application no. 14, columns 5 or 8.

for the disclosure of this paragraph see [0359.1.7.7] above

A nucleic acid molecule encoding a homolog to a polypeptide depicted in the respective line in Table II, application no. 14, columns 5 or 8 can be created by introducing one or more nucleotide substitutions, additions or deletions into the nucleic acid molecule of the present invention, in particular of Table I, application no. 14, columns 5 or 8, in the respective line, such that one or more amino acid substitutions, additions or deletions are introduced into the encoded protein. Mutations can be introduced into the nucleic acid molecules as depicted in the respective line in Table I, application no. 14, columns 5 or 8, preferably the coding region thereof, by standard techniques, such as site-directed mutagenesis, PCR-mediated mutagenesis or other methods known to the person skilled in the art.

[0361.1.7.14] to [0363.1.7.14] for the disclosure of these paragraphs see [0361.1.7.7] to [0363.1.7.7] above

Homologues of the nucleic acid sequences used, with the sequences shown in the respective line in Table I, application no. 14, columns 5 or 8, preferably the coding region thereof, comprise also allelic variants with at least approximately 30%, 35%, 40%, 45%, 50%, 55%, 60%, 65%, 70%, 75%, 80%, 85%, 90%, 91%, 92%, 93%, 94% 95%. 96%, 97%, 98%, 99%, 99.5% or more % homology with one of the nucleotide sequences shown or the abovementioned derived nucleic acid sequences or their homologues or derivatives or parts of these. Allelic variants encompass in particular functional variants which can be obtained by deletion, insertion or substitution of nucleotides from the sequences shown in the respective line, preferably in Table I, columns 5 or 8, preferably the coding region thereof, or from the derived nucleic acid sequences, the intention being, however, that the enzymatic activity or the biological activity of the resulting proteins synthesized is advantageously retained or increased.

In one embodiment of the present invention, the nucleic acid molecule of the invention or used in the process of the invention comprises the sequences shown in the respective line in any of the Table I, application no. 14, columns 5 or 8, preferably the coding region thereof, fragments or homologs thereof. It is preferred that the nucleic acid molecule comprises as little as possible other nucleotides not shown in the respective line in any one of Table I, application no. 14, columns 5 or 8, preferably the coding region thereof, fragments or homologs thereof. In one embodiment, the nucleic acid molecule comprises less than 500, 400, 300, 200, 100, 90, 80, 70, 60, 50 or 40 further nucleotides. In a further embodiment, the nucleic acid molecule comprises less than 30, 20 or 10 further nucleotides. In one embodiment, the nucleic acid molecule used in the process of the invention is identical to the sequences shown in the respective line in Table I, application no. 14, columns 5 or 8, preferably the coding region thereof.

Also preferred is that the nucleic acid molecule used in the process of the invention encodes a polypeptide comprising the sequence shown in the respective line in Table II, application no. 14, columns 5 or 8, fragments or homologs thereof. In one embodiment, the nucleic acid molecule encodes less than 150, 130, 100, 80, 60, 50, 40 or 30 further amino acids. In a further embodiment, the encoded polypeptide comprises less than 20, 15, 10, 9, 8, 7, 6 or 5 further amino acids. In one embodiment used in the inventive process, the encoded polypeptide is identical to the sequences shown in the respective line in Table II, application no. 14, columns 5 or 8.

In one embodiment, the nucleic acid molecule of the invention or used in the process encodes a polypeptide comprising the sequence shown in the respective line in Table II, application no. 14, columns 5 or 8, or fragments or homologs thereof, and comprises less than 100 further nucleotides. In a further embodiment, said nucleic acid molecule comprises less than 30 further nucleotides. In one embodiment, the nucleic acid molecule used in the process is identical to a coding sequence of the sequences shown in the respective line in Table I, application no. 14, columns 5 or 8.

Polypeptides (=proteins), which still have the essential biological or enzymatic activity of the polypeptide of the present invention conferring the production or the increased production of the respective fine chemical beta-sitosterol, campesterol, or stigmasterol as compared to a corresponding, e.g. non-transformed, wild type non-human organism, like a microorganism or a plant cell, plant or part thereof, i.e. whose activity is essentially not reduced, are polypeptides with at least 10%, 20%, 30%, 40%, 50%, 60%, 70%, 80%, 90%, 95% or more of the wild type biological activity or enzymatic activity, advantageously, the activity is essentially not reduced in comparison with the activity of a polypeptide shown in the respective line in Table II, application no. 14, columns 5 or 8 expressed under identical conditions.

Homologues of nucleic acid molecules shown in the respective line in Table I, application no. 14, columns 5 or 8, preferably the coding region thereof, or fragments thereof, or of the derived sequences shown in the respective line in Table II, application no. 14, columns 5 or 8, or fragments thereof, also mean truncated sequences, cDNA, single-stranded DNA or RNA of the coding and noncoding DNA sequence. Homologues of said sequences are also understood as meaning derivatives, which comprise noncoding regions such as, for example, UTRs, introns, terminators, enhancers or promoter variants. The promoters upstream of the nucleotide sequences stated can be modified by one or more nucleotide substitution(s), insertion(s) and/or deletion(s) without, however, interfering with the functionality or activity either of the promoters, the open reading frame (=ORF) or with the 3′-regulatory region such as terminators or other 3′-regulatory regions, which are far away from the ORF. It is furthermore possible that the activity of the promoters is increased by modification of their sequence, or that they are replaced completely by more active promoters, even promoters from heterologous organisms. Appropriate promoters are known to the person skilled in the art and are mentioned herein below.

[0370.1.7.14] to [0379.1.7.14] for the disclosure of these paragraphs see [0370.1.7.7] to [0379.1.7.7] above

Moreover, a native polypeptide conferring the increase of the respective fine chemical beta-sitosterol, campesterol, or stigmasterol in a non-human organism or a part thereof can be isolated from cells (e.g., endothelial cells), for example using the antibody of the present invention as described below, in particular, an antibody against proteins having 2-oxoglutarate dehydrogenase E1 subunit, ABC metal ion transporter substrate-binding protein, acetyl-CoA acetyltransferase, acetyltransferase, acid shock protein, acyl transferase, amidophosphoribosyltransferase, ankyrin repeat family protein, ATP-binding component of a transport system, b1401-protein, B1795-protein, b2107-protein, b2376-protein, b2399-protein, b2474-protein, b2513-protein, b2654-protein, b3151-protein, b3989-protein, b4029-protein, b4205-protein, C-4 sterol methyl oxidase, calcium-dependent protein kinase, cation transport protein, cation-transporting ATPase, CTP synthase, CTP synthetase, cyclin, cyclin D, eukaryotic translation initiation factor 5, glucose dehydrogenase, glucose-6-phosphate 1-dehydrogenase, glutaredoxin, homocitrate synthase, hydrolase, lipoprotein precursor, L-ribulose-5-phosphate 4-epimerase, membrane transport protein, methyltransferase, monothiol glutaredoxin, multidrug resistance protein, multiple antibiotic resistance protein, murein transglycosylase, NADH dehydrogenase I chain N, oxidoreductase, permease protein, permease protein of ABC transporter, phenylacetic acid degradation protein, phosphoribulokinase, polygalacturonase, protein kinase, protein phosphatase, putative transport system permease protein, putrescine transport system permease protein, recombinase A, sec-independent protein translocase, sll0406-protein, sll1761-protein, sodium/proton antiporter, sterol desaturase, threonine dehydrogenase, threonine synthase, transcription factor, tryptophan synthase alpha chain, TTC1386-protein, xanthine dehydrogenase, ydl235c-protein, ynl321w-protein, or ypr170c-protein activity, respectively, or an antibody against polypeptides as shown in the respective line in Table II, application no. 14, columns 5 or 8, or fragments or homologs thereof which can be produced by standard techniques utilizing the polypeptid of the present invention or fragment thereof, i.e., the polypeptide of this invention (FCRP). Preferred are monoclonal antibodies.

for the disclosure of this paragraph see [0381.1.7.7] above

In one embodiment, the present invention relates to a polypeptide as depicted in the respective line in Table II, especially in Table IIA or especially in Table IIB, application no. 14, columns 5 or 8, or fragment or homolog thereof or as coded by the nucleic acid molecule shown in the respective line in Table I, especially Table IA or especially Table IB, application no. 14, columns 5 or 8, preferably the coding region thereof, or fragment or homolog thereof.

In an embodiment, the present invention relates to a polypeptide comprising or consisting of a consensus sequence shown in the respective line in Table IV, application no. 14, column 8 or at least one polypeptide motif shown in the respective line in Table IV, application no. 14, column 8.

In an embodiment thereof, the present invention relates to a polypeptide comprising or consisting of a consensus sequence shown in the respective line in Table IV, application no. 14, column 8 or comprising at least one polypeptide motif shown in the respective line in Table IV, application no. 14, column 8, whereby not more than 20, 15, 10, 9, 8, 7, 6, 5, 4, 3, 2, or 1, or 0 of the amino acids can be replaced by any amino acid.

In another embodiment thereof, the present invention relates to a polypeptide comprising or consisting of a consensus sequence shown in the respective line in Table IV, application no. 14, column 8 or comprising at least one polypeptide motif shown in the respective line in Table IV, application no. 14, column 8, whereby not more than 15%, 10%, 5%, 4%, 3%, 2% or1% or 0% of the amino acids defined as distinct specific amino acids are/is replaced by another amino acid.

In another embodiment thereof, the present invention relates to a polypeptide comprising or consisting of a consensus sequence shown in the respective line in Table IV, application no. 14, column 8 or comprising at least one polypeptide motif shown in the respective line in Table IV, application no. 14, column 8, whereby not more than 20, 15, 10, 9, 8, 7, 6, 5, 4, 3, 2, or 1, or 0 amino acids are inserted into the consensus sequence or polypeptide motif.

In an embodiment of the present invention, the polypeptide of the present invention comprises or consists of a consensus sequence or at least one polypeptide motif shown in the respective line in Table IV, application no. 14, column 8, whereby said polypeptide distinguishes over the sequence depicted in the respective line in Table II, application no. 14, columns 5 or 8 by one or more amino acids. In one embodiment, said polypeptide of the invention distinguishes over the sequence shown in the respective line in Table II, application no. 14, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 14, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in the respective line in Table II, application no. 14, columns 5 and 8.

In an embodiment, the invention relates to a polypeptide conferring an increase in the respective fine chemical beta-sitosterol, campesterol, or stigmasterol in a non-human organism, especially a microorganism or a plant, or a part thereof, being encoded by the nucleic acid molecule of the invention or used in the process of the invention and having a sequence which distinguishes over the sequence as shown in the respective line in Table II, application no. 14, columns 5 or 8 by one or more amino acids (but not exceeding 5%, preferably 4%, 3%, 2%, 1%, 0.5%, 0.2% or 0.1%). In an embodiment, said polypeptide of the invention does not comprise or consist of the sequence shown in the respective line in Table II, application no. 14, columns 5 or 8. In an embodiment, said polypeptide of the present invention is less than 100%, 99.999%, 99.99%, 99.9% or 99% identical. In one embodiment, said polypeptide which differs at least in one or more amino acids (but not exceeding 5%, preferably 4%, 3%, 2%, 1%, 0.5%, 0.2% or 0.1%) from the polypeptide shown in the respective line in Table II, application no. 14, columns 5 and 8 does not comprise a protein of the sequence shown in the respective line in Table II A and/or II B, application no. 14, columns 5 or 8.

In an embodiment, the present invention relates to a polypeptide having the activity of the protein as shown in the respective line in Table II, application no. 14, column 3, which distinguishes over the sequence depicted in the respective line in Table II, application no. 14, columns 5 or 8 by one or more amino acids, preferably by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 14, columns 5 and 8 by not more than 80%, 70%, 60%, 50%, 40%, 30%, 20%, 10%, 5%, 4%, 3%, 2%, 1% or 0.5%.

In another embodiment the polypeptide of the invention takes the form of a preprotein consisting of a plastidial or mitochondrial transit peptide joint to a polypeptide having the activity of the protein as shown in Table II, column 3, from which the transit peptide is preferably cleaved off upon transport of the preprotein into the organelle for example into the plastid or mitochondrion.

In another embodiment the polypeptide of the invention takes not the form of a preprotein.

for the disclosure of this paragraph see [0390.1.7.7] above

for the disclosure of this paragraph see [0391.1.7.7] above

A polypeptide of the invention can participate in the process of the present invention. The polypeptide or a portion thereof comprises preferably an amino acid sequence, which is sufficiently homologous to an amino acid sequence shown in the respective line in Table II A and/or II B, application no. 14, columns 5 or 8.

Further, the polypeptide can have an amino acid sequence which is encoded by a nucleotide sequence which hybridizes, preferably hybridizes under stringent conditions as described above, to a nucleotide sequence of the nucleic acid molecule of the present invention. Accordingly, the polypeptide has an amino acid sequence which is encoded by a nucleotide sequence that is at least about 35%, 40%, 45%, 50%, 55%, 60%, 65%, 70%, 75%, 80%, 85%, 90%, 91%, 92%, 93%, 94%, 95%, 96%, 97%, 98%, 99%, 99.5% or more homologous to one of the amino acid sequences as shown in the respective line in Table II A and/or II B, application no. 14, columns 5 or 8, or fragments thereof. The preferred polypeptide of the present invention preferably possesses at least one of the activities according to the invention and described herein. A preferred polypeptide of the present invention includes an amino acid sequence encoded by a nucleotide sequence which hybridizes, preferably hybridizes under stringent conditions, to a nucleotide sequence shown in the respective line in Table I A and/or I B, application no. 14, columns 5 or 8, preferably the coding region thereof, or fragments thereto or which is homologous thereto, as defined above

The polypeptide of the present invention can vary from the sequences shown in the respective line in Table II A and/or II B, application no. 14, columns 5 or 8 in amino acid sequence due to natural variation or mutagenesis, as described in detail herein. Accordingly, the polypeptide comprise an amino acid sequence which is at least about 35%, 40%, 45%, 50%, 55%, 60%, 65%, 70%, 75%, 80%, 85% or 90, 91%, 92%, 93%, 94%, 95%, 96%, 97%, 98%, 99%, 99.5% or more homologous to an entire amino acid sequence shown in the respective line in Table II A and/or II B, application no. 14, columns 5 or 8.

Biologically active portions of a polypeptide of the present invention include peptides comprising amino acid sequences derived from the amino acid sequence of the polypeptide of the present invention or used in the process of the present invention, e.g., the amino acid sequence shown in the respective line in Table II, application no. 14, columns 5 or 8 or the amino acid sequence of a protein homologous thereto, which include fewer amino acids than a full length polypeptide of the present invention or used in the process of the present invention or the full length protein which is homologous to a polypeptide of the present invention or used in the process of the present invention depicted herein, and exhibit at least one activity of a polypeptide of the present invention or used in the process of the present invention.

for the disclosure of this paragraph see [0396.1.7.7] above

Manipulation of the nucleic acid molecule of the invention may result in the production of a protein having differences from the protein as shown in the respective line in Table II, application no. 14, column 5 or 8. Differences shall mean at least one amino acid different from the sequences as shown in the respective line in Table II, application no. 14, column 5 or 8, preferably at least 2, 3, 4, 5, 6, 7, 8, 9 or 10 amino acids (especially but not exceeding 0.1%, preferably 0.2%, 0.5%, 1%, 2%, 3% 4% or 5%), more preferably at least 15, 20, 25, 30, 35, 40, 45 or 50 amino acids (especially but not exceeding 0.5%, preferably 1%, 2%, 3% 4%, 5%, 10% or 25%) different from the sequences as shown in the respective line in Table II, application no. 14, column 5 or 8. These proteins may be improved in efficiency or activity, or may be more stable and therefore present in greater numbers in the cell as compared to the wild-type cell.

Any mutagenesis strategies for the polypeptide of the present invention or the polypeptide used in the process of the present invention to result in increasing said activity are not meant to be limiting; variations on these strategies will be readily apparent to one skilled in the art. Using such strategies, and incorporating the mechanisms disclosed herein, the nucleic acid molecule and polypeptide of the invention may be utilized to generate algae, ciliates, fungi, other microorganisms like C. glutamicum or plants or parts thereof, expressing proteins as shown in the respective line in Table II, application no. 14, column 5 or 8, or mutated proteins thereof. The nucleic acid molecules and polypeptide molecules of the invention are expressed such that the yield, production, and/or efficiency of production of a desired compound is improved.

[0399.1.7.14] to [0401.1.7.14] for the disclosure of these paragraphs see [0399.1.7.7] to [0401.1.7.7] above

In one embodiment, a protein (=polypeptide) as shown in the respective line in Table II, application no. 14, column 5 or 8 refers to a polypeptide having an amino acid sequence corresponding to the polypeptide of the invention, especially to a polypeptide as shown in Table II, application no. 14, columns 5 or 8 or homologs thereof, or used in the process of the invention, whereas a “non- inventive protein or polypeptide” or “other polypeptide” refers to a polypeptide having an amino acid sequence corresponding to a protein which is not substantially homologous to a polypeptide of the invention, preferably which is not substantially homologous to a polypeptide or protein as shown in the respective line in Table II, application no. 14, column 5 or 8 or homologs thereof, e.g., a protein which does not confer the activity described herein and which is derived from the same or a different organism.

[0403.1.7.14] to [0409.1.7.14] for the disclosure of these paragraphs see [0403.1.7.7] to [0409.1.7.7] above

As the nucleic acid molecules of the invention and/or the expression thereof is related to the synthesis of the respective fine chemical beta-sitosterol, campesterol, or stigmasterol its function as a probe extends to the detection of microorganisms, plant tissues, plants, plant variets, plant ecotypes or plant genera with varying, advantageously increased, capability or potential for synthesis of the respective fine chemical beta-sitosterol, campesterol, or stigmasterol. Therefore in one embodiment the present invention relates to a method for analyzing the capability or potential of a plant tissue, a plant, a plant variety or ecotype to produce the respective fine chemical beta-sitosterol, campesterol, or stigmasterol by using the respective antibody of the invention as a probe to detect the amount of the polypeptide encoded by said nucleic acid molecule of the invention in a non-human organism in comparison to another organism.

Therefore in one embodiment the invention relates to a method to probe a non-human organism for its capability for the production of the respective fine chemical comprising the steps of
a) analyzing the sequence and/or the expression of the respective nucleic acid molecule of the invention in least two non-human organisms which differ in the capability to produce the respective fine chemical;
b) associate the different sequence and/or expression level of the nucleic acid molecules of the invention with their different capability to produce the respective fine chemical;
c) analyze the sequence and/or the expression of the nucleic acid molecule of the invention in at least a third non-human organism;
d) predict the capability of the at least third organism to produce the respective fine chemical based by comparing the sequence and/or expression of the nucleic acid of the invention to the sequences and/or expression levels of the nucleic acid molecule of the invention of the at least two organisms analyzed in step a.

[0411.1.7.14] to [0430.1.7.14] for the disclosure of these paragraphs see [0411.1.7.7] t [0430.1.7.7] above

Accordingly the present invention relates to any cell transgenic for any nucleic acid characterized as part of the invention, e.g. conferring the increase of the respective fine chemical beta-sitosterol, campesterol, or stigmasterol in a cell or a non-human organism or a part thereof, e.g. the nucleic acid molecule of the invention, the nucleic acid construct of the invention, the vector of the invention, the expression cassette according to the invention, or a nucleic acid molecule encoding the polypeptide of the invention, e.g. encoding a polypeptide having an activity as the protein as shown in the respective line in Table II, application no. 14, column 3. Due to the above-mentioned activity the respective fine chemical beta-sitosterol, campesterol, or stigmasterol content in a cell or a non-human organism is increased. For example, due to modulation or manipulation, the cellular activity is increased, in a preferred embodiment in organelles such as plastids or mitochondria, e.g. due to an increased expression or specific activity or specific targeting of the subject matters of the invention in a cell or a non-human organism or a part thereof especially in organelles such as plastids or mitochondria, or in another embodiment in the cytosol. Transgenic for a polypeptide having a protein or a protein activity means herein that due to modulation or manipulation of the genome, the activity of protein as shown in the respective line in Table II, application no. 14, column 3 or a protein as shown in the respective line in Table II, application no. 14, column 3-like activity is increased in the cell or non-human organism or part thereof, especially in organelles such as plastids or mitochondria, or especially in the cytosol. Examples are described above in context with the process of the invention.

for the disclosure of this paragraph see [0432.1.7.7] above

A naturally occurring expression cassette—for example the naturally occurring combination of the promoter of the gene encoding a protein as shown in the respective line in Table II, application no. 14, column 5 or 8, preferably the coding region thereof, or fragments or homologs thereof with the corresponding encoding gene—becomes a transgenic expression cassette when it is modified by non-natural, synthetic “artificial” methods such as, for example, mutagenesis. Such methods have been described (U.S. Pat. No. 5,565,350; WO 00/15815; also see above).

for the disclosure of this paragraph see [0434.1.7.7] above

for the disclosure of this paragraph see [0435.1.7.7] above

Transgenic plants comprising the respective fine chemical(s) synthesized in the process according to the invention can be marketed directly without isolation of the fine chemical synthesized. In the process according to the invention, plants are understood as meaning all plant parts, plant organs such as leaf, stalk, root, tubers or seeds or propagation material or harvested material or the intact plant. In this context, the seed encompasses all parts of the seed such as the seed coats, epidermal cells, seed cells, endosperm or embryonic tissue. The fine chemical produced in the process according to the invention may, however, also be isolated from the plant. In case of beta-sitosterol, campesterol, or stigmasterol this can be in free form or bound to proteins. Fine chemical(s) produced by this process can be harvested by harvesting the non-human organisms either from the culture in which they grow or from the field. For example, this can be done via squeezing, grinding and/or extraction, salt precipitation and/or ion-exchange chromatography of the plant parts, preferably the plant seeds, plant fruits, plant tubers and the like.

[0437.1.7.14] to [0447.1.7.14] for the disclosure of these paragraphs see [0437.1.7.7] to [0447.1.7.7] above

Identity and purity of the phytosterols such as beta-sitosterol, stigmasterol and/or campesterol isolated can be determined by standard techniques of the art. They encompass old techniques such as photometric methods and new techniques such as high-performance liquid chromatography (HPLC), gas chromatography (GC), spectroscopic methods, mass spectrometry (MS), staining methods, thin-layer chromatography, NIRS, enzyme assay or microbiological assays.

Phytosterols can be detected via HPLC, e.g. reversed-phase HPLC, as described by Heftmann, E. and Hunter, I.R. (J Chromatogr 1979; 165: 283-299. Furthermore the analysis of phytosterols can be performed as described in S. L. Abidi, “Chromatographic analysis of plant sterols in foods and vegetable oils [Review]”, Journal of Chromatography 2001, A.. 935, 173-20 or P. Appelblad, K. Irgum, “Separation and detection of neuroactive steroids from biological matrices [Review]”, Journal of Chromatography A 2002, 955, 151-182. or K. Shimada, K. Mitamura, T. Higashi, “Gas chromatography and high-performance liquid chromatography of natural steroids [Review]”, Journal of Chromatography 2001, A.. 935, 141-172.

For the sterol isolation and analysis the German standard method F III may be used. The method comprises: saponification of fat, isolation of unsaponifiable matter using an aliminium oxide column, separation of sterol fraction by preparative TLC and determination of the composition of sterols as trimetysilyl ethers by GC.

The phytosterols obtained in the process are suitable as starting material for the synthesis of further products of value. For example, phytosterols are efficient cholesterol-lowering agents leading to the prevention of cardiovascular disease. In addition, they produce a wide spectrum of therapeutic effects including anti-tumor properties. Epidemiologic and experimental studies suggest that dietary phytosterols, preferably beta-sitosterol, stigmasterol and/or campesterol may offer protection from the most common cancers in Western societies, such as colon, breast and prostate cancer.

Accordingly, the present invention relates a method for the production of a pharmaceuticals, food stuff, animal feeds, nutrients or cosmetics comprising the steps of the process according to the invention, including the isolation of the fine chemical produced or a composition produced comprising the fine chemical, and, if desired, formulating the product with a pharmaceutical acceptable carrier or formulating the product in a form acceptable for an application in agriculture or formulating the product in a form acceptable for an application as food stuff, animal feed, nutrient or cosmetic. A further embodiment according to the invention is the use of the fine chemical produced in the process or of the transgenic non-human organisms in animal feeds, foodstuffs, medicines, food supplements, cosmetics or pharmaceuticals. %% ERROR %%.

for the disclosure of this paragraph see [0450.1.7.7] above

for the disclosure of this paragraph see [0451.1.7.7] above

Further in another embodiment, the present invention relates to the use of the nucleic acid molecule of the invention or used in the method of the invention alone or in combination with other genes of the respective fine chemical synthesis for example of the phytosterol biosynthesis, the polypeptide of the invention or used in the method of the invention, the nucleic acid construct of the invention, the expression cassette of the invention, the vector of the invention, the plant or plant tissue or the host cell of the invention, for the production of plant resistant to a herbicide inhibiting eventually the production of the fine chemical.

for the disclosure of this paragraph see [0453.1.7.7] above

for the disclosure of this paragraph see [0454.1.7.7] above

In one embodiment, the present invention relates to a method for the identification of a gene product conferring the production of or an increase in the fine chemical production in a non-human cell, comprising the following steps:
(a) contacting, e.g. hybridizing, a, some or all nucleic acid molecules of a sample, e.g. cells, tissues, plants or microorganisms or a nucleic acid library, which can contain a candidate gene encoding a gene product conferring the production of or an increase in the fine chemical after expression, with the nucleic acid molecule of the present invention as shown in the respective line in column 5 or 8 of Table I A or B of application no. 14, preferably the coding region thereof, or a fragment or homolog thereof;
(b) identifying the nucleic acid molecules, which hybridize under relaxed stringent conditions with the nucleic acid molecule of the present invention in particular to the nucleic acid molecule sequence shown in the respective line in Table I, application no. 14, columns 5 or 8, preferably the coding region thereof, of a fragment or a homolog thereof, preferably in Table I B, application no. 14, columns 5 or 8, preferably the coding region thereof, of a fragment or a homolog thereof, and, optionally, isolating the full length cDNA clone or complete genomic clone;
(c) introducing the candidate nucleic acid molecules or a fragment thereof in host cells, preferably in a plant cell or a microorganism, appropriate for producing the fine chemical;
(d) expressing the identified nucleic acid molecules in the host cells for which the production of or the increased production of the fine chemical is desired;
(e) assaying the fine chemical level in the non-human host cells; and
(f) identifying the nucleic acid molecule and/or its gene product which expression confers the production of or an increase in the fine chemical level in the non-human host cell after expression compared to the wild type.

for the disclosure of this paragraph see [0456.1.7.7] above

In another embodiment, the present invention relates to a method for the identification of a gene product conferring the production of or an increase in the fine chemical production in a cell, comprising the following steps:
(a) identifying a nucleic acid molecule of a non-human organism, which is at least 20%, 25%, 30%, 35%, 40%, 50%, 60%, 70%, 80%, 90%, 95%, 96%, 97%, 98%, 99%, 99.5% or more homolog to the nucleic acid molecule encoding a polypeptide comprising the polypeptide molecule as shown in the respective line in column 5 or 8 of Table II, application no. 14, or comprising a consensus sequence or a polypeptide motif as shown in the respective line in column 8 of Table IV, application no. 14, or being encoded by a nucleic acid molecule comprising a polynucleotide as shown in the respective line in column 5 or 8 of Table I, application no. 14, preferably the coding region thereof, or a homologue thereof as described herein, for example via homology search in a data bank;
(b) introducing the candidate nucleic acid molecule in non-human host cells, preferably in a plant cell or a microorganism, appropriate for producing the fine chemical;
(c) expressing the identified nucleic acid molecule in the non-human host cells;
(d) assaying the fine chemical level in the non-human host cells; and
(e) identifying the nucleic acid molecule and its gene product which expression confers the production of or an increase in the fine chemical level in the non-human host cell after expression compared to the wild type.

for the disclosure of this paragraph see [0458.1.7.7] above

Further, the nucleic acid molecules disclosed herein, in particular the nucleic acid molecules shown in the respective line in column 5 or 8 of Table I A or B of application no. 14, may be sufficiently homologous to the sequences of related species such that these nucleic acid molecules may serve as markers for the construction of a genomic map in related organism or for association mapping. Furthermore natural variation in the genomic regions corresponding to nucleic acids disclosed herein, in particular the nucleic acid molecule shown in the respective line in column 5 or 8 of Table I A or B, of application no. 14 or homologous thereof, may lead to variation in the activity of the proteins disclosed herein, in particular the proteins comprising polypeptides as shown in the respective lines in column 5 or 8 of Table II A or B, of application no. 14 or their homologous, or comprising the consensus sequence or the polypeptide motif as shown in the respective line in column 8 of Table IV, and in consequence in a natural variation of an increased fine chemical production.

In consequence natural variation eventually also exists in form of more active allelic variants leading already to a relative increase in the fine chemical. Different variants of the nucleic acid molecules disclosed herein, in particular the nucleic acid molecules comprising the nucleic acid molecules as shown in the respective line in column 5 or 8 of Table I A or B of application no. 14, preferably the coding region thereof, which corresponds to different levels of increase in fine chemical, e.g. different levels of increase in fine chemical, can be indentified and used for marker assisted breeding for an enhanced production of the fine chemical.

Natural variation may also exist in the regulatory regions, e.g. the promoters of the natural genes comprising the nucleic acid molecules as shown in the respective line column 5 or 8 of Table I A or B of application no. 14, preferably the coding region thereof, which can similarly correspond to different levels of increase in the fine chemical, e.g. different levels of increase in the fine chemical can be identified and used for marker assisted breeding for an enhanced production of the fine chemical.

Accordingly, the present invention relates to a method for breeding plants for the production of the fine chemical, comprising
(a) selecting a first plant variety with an increased production of the fine chemical, e.g. based on an increased expression of a nucleic acid molecule of the invention as disclosed herein, in particular of a nucleic acid molecule comprising a nucleic acid molecule as shown in the respective line in column 5 or 8 of Table I A or B of application no. 14, preferably the coding region thereof, or fragments or homologs thereof, or a polypeptide comprising a polypeptide as shown in the respective line in column 5 or 8 of Table II A or B of application no. 14, or fragments or homologs thereof, or comprising a consensus sequence or a polypeptide motif as shown in the respective line in column 8 of Table IV, or a homolog thereof, as described herein;
(b) associating the level of increased production of the fine chemical, with the expression level or the genomic structure of a gene encoding said polypeptide or said nucleic acid molecule;
(c) crossing the first plant variety with a second plant variety, which significantly differs in its level of fine chemical; and
(d) identifying, which of the offspring varieties has got increased production of the fine chemical by the expression level of said polypeptide or nucleic acid molecule or the genomic structure of the gene(s) encoding said polypeptide or nucleic acid molecule of the invention.

[0462.1.7.14] to [0479.1.7.14] for the disclosure of these paragraphs see [0462.1.7.7] to [0479.1.7.7] above

In a further embodiment the nucleic acid molecule of the invention, the polypeptide of the invention, the nucleic acid construct of the invention, the expression cassette of the invention, the non-human organisms, the non-human host cell, the microorganisms, the plant, plant cell, or the part thereof, of the invention, the vector of the invention, the agonist identified with the method of the invention, the nucleic acid molecule identified with the method of the present invention, can be used for the production of the fine chemical or of the fine chemical and one or more other phytosterols.

Accordingly, the nucleic acid molecule of the invention, or the nucleic acid molecule identified with the method of the present invention or the complement sequences thereof, the polypeptide of the invention, the nucleic acid construct of the invention, the expression cassette of the invention, the non-human organism, the non-human host cell, the microorganism, the plant, plant cell, or the part thereof of the invention, the vector of the invention, the agonist identified with the method of the invention, the antibody of the present invention, can be used for the production of or the increase in the fine chemical in a non-human organism or part thereof, e.g. in a cell.

for the disclosure of this paragraph see [0481.1.7.7] above

for the disclosure of this paragraph see [0482.1.7.7] above

A special embodiment of this invention is related to genes which increase or generate the production of the fine chemical beta-sitosterol, campesterol, and/or stigmasterol in plant cells, plants or part thereof. Phenotypes thereto are associated with yield of plants (=yield related phenotypes). In accordance with the invention, therefore, the respective genes identified in Table I, wherein in column 7 beta-sitosterol, campesterol, and/or stigmasterol are mentioned, especially the coding region thereof, or homologs or fragments thereof,may be employed to enhance any yield-related phenotype. Increased yield may be determined in field trials of transgenic plants and suitable control plants. Alternatively, a transgene's ability to increase yield may be determined in a model plant. An increased yield phenotype may be determined in the field test or in a model plant by measuring any one or any combination of the following phenotypes, in comparison to a control plant: yield of dry harvestable parts of the plant, yield of dry aerial harvestable parts of the plant, yield of underground dry harvestable parts of the plant, yield of fresh weight harvestable parts of the plant, yield of aerial fresh weight harvestable parts of the plant yield of underground fresh weight harvestable parts of the plant, yield of the plant's fruit (both fresh and dried), grain dry weight, yield of seeds (both fresh and dry), and the like.

The most basic yield-related phenotype is increased yield associated with the presence of the gene or a homolog or a fragment thereof as a transgene in the plant, i.e., the intrinsic yield of the plant. Intrinsic yield capacity of a plant can be, for example, manifested in a field test or in a model system by demonstrating an improvement of seed yield (e.g. in terms of increased seed/grain size, increased ear number, increased seed number per ear, improvement of seed filling, improvement of seed composition, embryo and/or endosperm improvements, and the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under non-stressed conditions), improvement in plant architecture.

Increased yield-related phenotypes may also be measured to determine tolerance to abiotic environmental stress. Abiotic stresses include drought, low temperature, salinity, osmotic stress, shade, high plant density, mechanical stresses, and oxidative stress, and yield-related phenotypes are encompassed by tolerance to such abiotic stresses. Additional phenotypes that can be monitored to determine enhanced tolerance to abiotic environmental stress include, without limitation, wilting; leaf browning; loss of turgor, which results in drooping of leaves or needles stems, and flowers; drooping and/or shedding of leaves or needles; the leaves are green but leaf angled slightly toward the ground compared with controls; leaf blades begun to fold (curl) inward; premature senescence of leaves or needles; loss of chlorophyll in leaves or needles and/or yellowing. Any of the yield-related phenotypes described above may be monitored in field tests or in model plants to demonstrate that a transgenic plant has increased tolerance to abiotic environmental stress. In accordance with the invention, the respective genes identified in Table 1, in case in column 7 beta-sitosterol, campesterol, and/or stigmasterol is indicated, especially the coding region thereof, or homologs or fragments thereof, may be employed to enhance tolerance to abiotic environmental stress in a plant means that the plant, when confronted with abiotic environmental stress.

A polypeptide conferring an yield-increasing activity can be encoded by a respective nucleic acid sequence as shown in Table I, column 5 or 8, and/or comprises or consists of a respective polypeptide as depicted in Table II, column 5 and 8, and/or can be amplified with the respective primer set shown in Table III, column 8, in case in column 7 beta-sitosterol, campesterol, and/or stigmasterol is indicated.

“Improved adaptation” to environmental stress like e.g. freezing and/or chilling temperatures refers to an improved plant performance under environmental stress conditions.

A modification, i.e. an increase, can be caused by endogenous or exogenous factors. For example, an increase in activity in an organism or a part thereof can be caused by adding a gene product or a precursor or an activator or an agonist to the media or nutrition or can be caused by introducing said subjects into an organism, transient or stable. Furthermore such an increase can be reached by the introduction of the respective inventive nucleic acid sequence or the encoded protein in the correct cell compartment for example into the nucleus or cytoplasmic respectively or into plastids either by transformation and/or targeting.

The term “yield” as used herein generally refers to a measurable produce from a plant, particularly a crop. Yield and yield increase (in comparison to a non-transformed starting or wild-type plant) can be measured in a number of ways, and it is understood that a skilled person will be able to apply the correct meaning in view of the particular embodiments, the particular crop concerned and the specific purpose or application concerned. The terms “improved yield” or “increased yield” can be used interchangeable.

As used herein, the term “improved yield” or the term “increased yield” means any improvement in the yield of any measured plant product, such as grain, fruit or fiber. In accordance with the invention, changes in different phenotypic traits may improve yield. For example, and without limitation, parameters such as floral organ development, root initiation, root biomass, seed number, seed weight, harvest index, tolerance to abiotic environmental stress, leaf formation, phototropism, apical dominance, and fruit development, are suitable measurements of improved yield. Any increase in yield is an improved yield in accordance with the invention. For example, the improvement in yield can comprise a 0.1%, 0.5%, 1%, 3%, 5%, 10%, 15%, 20%, 30%, 40%, 50%, 60%, 70%, 80%, 90% or greater increase in any measured parameter. For example, an increase in the bushel/acre yield of soybeans or corn derived from a crop comprising plants which are transgenic for the nucleotides and polypeptides of Table I, in case in column 7 beta-sitosterol, campesterol, and/or stigmasterol is mentioned, as compared with the bushel/acre yield from untreated soybeans or corn cultivated under the same conditions, is an improved yield in accordance with the invention. The increased or improved yield can be achieved in the absence or presence of stress conditions.

For example, enhanced or increased “yield” refers to one or more yield parameters selected from the group consisting of biomass yield, dry biomass yield, aerial dry biomass yield, underground dry biomass yield, fresh-weight biomass yield, aerial fresh-weight biomass yield, underground fresh-weight biomass yield; enhanced yield of harvestable parts, either dry or freshweight or both, either aerial or underground or both; enhanced yield of crop fruit, either dry or fresh-weight or both, either aerial or underground or both; and preferably enhanced yield of seeds, either dry or fresh-weight or both, either aerial or underground or both. Crop yield is defined herein as the number of bushels of relevant agricultural product (such as grain, forage, or seed) harvested per acre. Crop yield is impacted by abiotic stresses, such as drought, heat, salinity, and cold stress, and by the size (biomass) of the plant. Traditional plant breeding strategies are relatively slow and have in general not been successful in conferring increased tolerance to abiotic stresses. Grain yield improvements by conventional breeding have nearly reached a plateau in maize.

Accordingly, the yield of a plant can depend on the specific plant/crop of interest as well as its intended application (such as food production, feed production, processed food production, biofuel, biogas or alcohol production, or the like) of interest in each particular case. Thus, in one embodiment, yield is calculated as harvest index (expressed as a ratio of the weight of the respective harvestable parts divided by the total biomass), harvestable parts weight per area (acre, square meter, or the like); and the like. The harvest index, i.e., the ratio of yield biomass to the total cumulative biomass at harvest, in maize has remained essentially unchanged during selective breeding for grain yield over the last hundred years. Accordingly, recent yield improvements that have occurred in maize are the result of the increased total biomass production per unit land area. This increased total biomass has been achieved by increasing planting density, which has led to adaptive phenotypic alterations, such as a reduction in leaf angle, which may reduce shading of lower leaves, and tassel size, which may increase harvest index. Harvest index is relatively stable under many environmental conditions, and so a robust correlation between plant size and grain yield is possible. Plant size and grain yield are intrinsically linked, because the majority of grain biomass is dependent on current or stored photosynthetic productivity by the leaves and stem of the plant. As with abiotic stress tolerance, measurements of plant size in early development, under standardized conditions in a growth chamber or greenhouse, are standard practices to measure potential yield advantages conferred by the presence of a transgene.

Acccordingly, the yield of a plant can be increased by improving one or more of the yield-related phenotypes.

Such yield-related phenotypes or traits of a plant the improvement of which results in increased yield comprise, without limitation, the increase of the intrinsic yield capacity of a plant, improved nutrient use efficiency, and/or increased stress tolerance.

For example, yield refers to biomass yield, e.g. to dry weight biomass yield and/or fresh-weight biomass yield. Biomass yield refers to the aerial or underground parts of a plant, depending on the specific circumstances (test conditions, specific crop of interest, application of interest, and the like). In one embodiment, biomass yield refers to the aerial and underground parts. Biomass yield may be calculated as fresh-weight, dry weight or a moisture adjusted basis. Biomass yield may be calculated on a per plant basis or in relation to a specific area (e.g. biomass yield per acre/ square meter/ or the like).

In other embodiment, “yield” refers to seed yield which can be measured by one or more of the following parameters: number of seeds or number of filled seeds (per plant or per area (acre/square meter/ or the like)); seed filling rate (ratio between number of filled seeds and total number of seeds); number of flowers per plant; seed biomass or total seeds weight (per plant or per area (acre/square meter/ or the like); thousand kernel weight (TKW; extrapolated from the number of filled seeds counted and their total weight; an increase in TKW may be caused by an increased seed size, an increased seed weight, an increased embryo size, and/or an increased endosperm). Other parameters allowing to measure seed yield are also known in the art. Seed yield may be determined on a dry weight or on a fresh weight basis, or typically on a moisture adjusted basis, e.g. at 15.5 percent moisture.

For example, the term “increased yield” means that the plant, exhibits an increased growth rate, under conditions of abiotic environmental stress, compared to the corresponding wild-type plant.

An increased growth rate may be reflected inter alia by or confers an increased biomass production of the whole plant, or an increased biomass production of the aerial parts of a plant, or by an increased biomass production of the underground parts of a plant, or by an increased biomass production of parts of a plant, like stems, leaves, blossoms, fruits, and/or seeds.

Increased yield includes higher fruit yields, higher seed yields, higher fresh matter production, and/or higher dry matter production.

The term “increased yield” means that the plant, exhibits a prolonged growth under conditions of abiotic environmental stress, as compared to the corresponding, e.g. non-transformed, wild type organism. A prolonged growth comprises survival and/or continued growth of the plant, at the moment when the non-transformed wild type organism shows visual symptoms of deficiency and/or death.

When the plant of the invention is a corn plant increased yield for corn plants means, for example, increased seed yield, in particular for corn varieties used for feed or food. Increased seed yield of corn refers to an increased kernel size or weight, an increased kernel per ear, or increased ears per plant. Alternatively or in addition the cob yield may be increased, or the length or size of the cob is increased, or the kernel per cob ratio is improved.

When the plant of the invention is a soy plant, increased yield for soy plants means increased seed yield, in particular for soy varieties used for feed or food. Increased seed yield of soy refers for example to an increased kernel size or weight, an increased kernel per pod, or increased pods per plant.

When the plant of the invention is an oil seed rape (OSR) plant, increased yield for OSR plants means increased seed yield, in particular for OSR varieties used for feed or food. Increased seed yield of OSR refers to an increased seed size or weight, an increased seed number per silique, or increased siliques per plant.

When the plant of the invention is a cotton plant,increased yield for cotton plants means increased lint yield. Increased lint yield of cotton refers in one embodiment to an increased length of lint.

When the plant of the invention is a wheat plant increased yield for wheat plants means, for example, increased seed yield, in particular for wheat varieties used for feed or food. Increased seed yield of wheat refers to an increased kernel size or weight, an increased kernel per ear, or increased ears per plant. Increased seed yield of wheat refers also to an increased grain size or weight, an increased number of grains per spike, or increased number of spikes per plant. Alternatively or in addition increased seed yield of wheat refers also to an increased grain yield, increased length of spikes, increased number of spikelets per spike , increased 1000 grain weight or increased flag leaf area (flag leaf is the leaf directely below the spike).

Said increased yield can typically be achieved by enhancing or improving, one or more yieldrelated traits of the plant. Such yield-related traits of a plant comprise, without limitation, the increase of the intrinsic yield capacity of a plant, improved nutrient use efficiency, and/or increased stress tolerance, in particular increased abiotic stress tolerance.

Intrinsic yield capacity of a plant can be, for example, manifested by improving the specific (intrinsic) seed yield (e.g. in terms of increased seed/grain size, increased ear number, increased seed number per ear, improvement of seed filling, improvement of seed composition, embryo and/or endosperm improvements, or the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under stressed or non-stressed conditions), improvement in plant architecture, cell cycle modifications, photosynthesis modifications, various signaling pathway modifications, modification of transcriptional regulation, modification of translational regulation, modification of enzyme activities, and the like); and/or the like.

The improvement or increase of stress tolerance of a plant can for example be manifested by improving or increasing a plant's tolerance against stress, particularly abiotic stress. In the present application, abiotic stress refers generally to abiotic environmental conditions a plant is typically confronted with, including, but not limited to, drought (tolerance to drought may be achieved as a result of improved water use efficiency), heat, low temperatures and cold conditions (such as freezing and chilling conditions), salinity, osmotic stress , shade, high plant density, mechanical stress, oxidative stress, and the like.

The increased plant yield can also be mediated by increasing the “nutrient use efficiency of a plant”, e.g. by improving the use efficiency of nutrients including, but not limited to, phosphorus, potassium, and nitrogen. Further, higher yields may be obtained with current or standard levels of nitrogen use.

Generally, the term “increased tolerance to stress” can be defined as survival of plants, and/or higher yield production, under stress conditions as compared to a non-transformed wild type or starting plant: For example, the plant of the invention or produced according to the method of the invention is better adapted to the stress conditions. “Improved adaptation” to environmental stress like e.g. drought, heat, nutrient depletion, freezing and/or chilling temperatures refers herein to an improved plant performance resulting in an increased yield, particularly with regard to one or more of the yield related traits as defined in more detail above.

During its life-cycle, a plant is generally confronted with a diversity of environmental conditions. Any such conditions, which may, under certain circumstances, have an impact on plant yield, are herein referred to as “stress” condition. Environmental stresses may generally be divided into biotic and abiotic (environmental) stresses. Unfavorable nutrient conditions are sometimes also referred to as “environmental stress”. The present invention does also contemplate solutions for this kind of environmental stress, e.g. referring to increased nutrient use efficiency.

For the purposes of the description of the present invention, the terms “enhanced tolerance to abiotic stress”, “enhanced resistance to abiotic environmental stress”, “enhanced tolerance to environmental stress”, “improved adaptation to environmental stress” and other variations and expressions similar in its meaning are used interchangeably and refer, without limitation, to an improvement in tolerance to one or more abiotic environmental stress(es) as described herein and as compared to a corresponding origin or wild type plant or a part thereof.

The term abiotic stress tolerance(s) refers for example low temperature tolerance, drought tolerance or improved water use efficiency (WUE), heat tolerance, salt stress tolerance and others. Studies of a plant's response to desiccation, osmotic shock, and temperature extremes are also employed to determine the plant's tolerance or resistance to abiotic stresses. Water use efficiency (WUE) is a parameter often correlated with drought tolerance. In selecting traits for improving crops, a decrease in water use, without a change in growth would have particular merit in an irrigated agricultural system where the water input costs were high. An increase in growth without a corresponding jump in water use would have applicability to all agricultural systems. In many agricultural systems where water supply is not limiting, an increase in growth, even if it came at the expense of an increase in water use also increases yield.

Drought stress means any environmental stress which leads to a lack of water in plants or reduction of water supply to plants, including a secondary stress by low temperature and/or salt, and/or a primary stress during drought or heat, e.g. desiccation etc.

### Specific Embodiments

Accordingly, this invention provides respective measures and methods to produce plants with increased yield, e.g. genes conferring an increased yield-related trait, for example enhanced tolerance to abiotic environmental stress, for example an increased drought tolerance and/or low temperature tolerance and/or an increased nutrient use efficiency, intrinsic yield and/or another increased yield-related trait, upon expression or over-expression. Accordingly, the present invention provides such genes in case in column 7 of the respective Table or beta-sitosterol, campesterol, and/or stigmasterol is indicated. In particular, such genes are described in column 5 as well as in column 8 of Tables I, especially the coding region thereof, or homologs or fragments thereof, in case beta-sitosterol, campesterol, and/or stigmasterol are indicated in column 7 or the respective polypeptides are described in column 5 as well as in column 8 of Table II, or homologs or fragments thereof, in case beta-sitosterol, campesterol, and/or stigmasterol are indicated in column 7.

Accordingly, the present invention provides respective transgenic plants showing one or more improved yield-related traits as compared to the corresponding origin or the wild type plant and methods for producing such transgenic plants with increased yield in case in column 7 of the respective Table “beta-sitosterol” or “campesterol” or “stigmasterol”is indicated. In one embodiment, one or more of said yield-increasing activities are increased by increasing the amount and/or the specific activity of one or more proteins listed in Table II, column 5 or 8 in a compartment of a cell indicated in Table II, column 6, in case in column 7 of the respective Table “beta-sitosterol” or “campesterol” or “stigmasterol” is indicated.

Accordingly to present invention, the yield of the plant of the invention is increased by improving one or more of the yield-related traits as defined herein. Said increased yield in accordance with the present invention can typically be achieved by enhancing or improving, in comparison to an origin or wild-type plant, one or more yield-related traits of said plant. Such yield-related traits of a plant the improvement of which results in increased yield comprise, without limitation, the increase of the intrinsic yield capacity of a plant, improved nutrient use efficiency, like nitrogen use efficiency, and/or increased stress tolerance.

In one embodiment, the present invention provides respective transgenic plants showing one or more improved yield-related traits, preferably increased drought tolerance as compared to the corresponding origin or the wild type plant and methods for producing such transgenic plants with increased yield in case in column 7 of the respective Table “beta-sitosterol” or “campesterol” or “stigmasterol”is indicated.

In one embodiment, the present invention provides respective transgenic plants showing one or more improved yield-related traits, preferably increased stress tolerance against low temperatures and cold conditions (such as freezing and chilling conditions) as compared to the corresponding origin or the wild type plant and methods for producing such transgenic plants with increased yield in case in column 7 of the respective Table “beta-sitosterol” or “campesterol” is indicated.

In a further embodiment the present invention is characterized by the features as disclosed in the following items:

Item 1. A process for the production of a fine chemical selected from the group consisting of: beta-sitosterol, campesterol and/or stigmasterol
(a) increasing or generating one or more activities selected from the group consisting of 2-oxoglutarate dehydrogenase E1 subunit, ABC metal ion transporter substratebinding protein, acetyl-CoA acetyltransferase, acetyltransferase, acid shock protein, acyl transferase, amidophosphoribosyltransferase, ankyrin repeat family protein, ATP-binding component of a transport system, b1401-protein, B1795-protein, b2107-protein, b2376-protein, b2399-protein, b2474-protein, b2513-protein, b2654-protein, b3151-protein, b3989-protein, b4029-protein, b4205-protein, C-4 sterol methyl oxidase, calcium-dependent protein kinase, cation transport protein, cation-transporting ATPase, CTP synthase, CTP synthetase, cyclin, cyclin D, eukaryotic translation initiation factor 5, glucose dehydrogenase, glucose-6-phosphate 1-dehydrogenase, glutaredoxin, homocitrate synthase, hydrolase, lipoprotein precursor, L-ribulose-5-phosphate 4-epimerase, membrane transport protein, methyltransferase, monothiol glutaredoxin, multidrug resistance protein, multiple antibiotic resistance protein, murein transglycosylase, NADH dehydrogenase I chain N, oxidoreductase, permease protein, permease protein of ABC transporter, phenylacetic acid degradation protein, phosphoribulokinase, polygalacturonase, protein kinase, protein phosphatase, putative transport system permease protein, putrescine transport system permease protein, recombinase A, sec-independent protein translocase, sll0406-protein, sll1761-protein, sodium/proton antiporter, sterol desaturase, threonine dehydrogenase, threonine synthase, transcription factor, tryptophan synthase alpha chain, TTC1386-protein, xanthine dehydrogenase, ydl235c-protein, ynl321w-protein, and ypr170c-protein, in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of beta-sitosterol, campesterol and/or stigmasterol or a composition comprising beta-sitosterol, campesterol and/or stigmasterol in said non-human organism or in the culture medium surrounding said non-human organism.

Item 2. A process for the production of a fine chemical selected from the group consisting of: beta-sitosterol, campesterol and/or stigmasterol, which comprises
(A)
   (i) increasing or generating of the expression of; and/or
   (ii) increasing or generating the expression of an expression product of; and/or
   (iii) increasing or generating one or more activities of an expression product encoded by;
      at least one respective nucleic acid molecule comprising a nucleic acid molecule selected from the group consisting of:
   (a) a nucleic acid molecule encoding the polypeptide shown in column 5 or 8 of Table II, preferably Table II B, application no.14, or a homolog or a fragment thereof;
   (b) a nucleic acid molecule shown in column 5 or 8 of Table I, preferably Table I B, application no.14, or a homolog or a fragment thereof (preferably the coding region thereof);
   (c) a nucleic acid molecule, which, as a result of the degeneracy of the genetic code, can be derived from a polypeptide sequence depicted in column 5 or 8 of Table II, preferably Table II B, application no.14;
   (d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 14, or the coding region thereof;
   (e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no.14;
   (i) a nucleic acid molecule encoding a polypeptide having the activity represented by a respective protein as depicted in column 5 of Table II, application no. 14;
   (j) a nucleic acid molecule which comprises a polynucleotide, which is obtained by amplifying a cDNA library or a genomic library using the primers as depicted in column 8 of Table III, application no. 14; and
   (k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
   or a nucleic acid molecule comprising a sequence which is complementary thereto; in a non-human organism or a part thereof, preferably a microorganism, a plant cell, a plant or a part thereof, as compared to a corresponding non-transformed wild type non-human organism or a part thereof; and
(B) growing the non-human organism or a part thereof under conditions which permit the production of beta-sitosterol, campesterol and/or stigmasterol or a composition comprising beta-sitosterol, campesterol and/or stigmasterol in said non-human organism or in the culture medium surrounding said non-human organism.

Item 3. A process of items 1 or 2, comprising of recovering beta-sitosterol, campesterol and/or stigmasterol in its free or bound form.

Item 4. The process of any one of items 1 to 3, comprising the folowing steps:
(a) selecting a non-human organism or a part thereof expressing a polypeptide encoded by the nucleic acid molecule characterized in item 2;
(b) mutagenizing the selected non-human organism or the part thereof;
(c) comparing the activity or the expression level of said polypeptide in the mutagenized non-human organism or the part thereof with the activity or the expression of said polypeptide of the selected non-human organisms or the part thereof;
(d) selecting the mutated non-human organisms or parts thereof, which comprise an increased activity or expression level of said polypeptide compared to the selected non-human organism or the part thereof;
(e) optionally, growing and cultivating the non-human organisms or the parts thereof; and
(f) recovering, and optionally isolating, the free or bound beta-sitosterol, campesterol and/or stigmasterol produced by the selected mutated non-human organisms or parts thereof.

Item 5. The process of any one of items 1 to 4 wherein the activity of said protein or the expression of said nucleic acid molecule is increased or generated transiently or stably.

Item 6. An isolated nucleic acid molecule comprising a nucleic acid molecule selected from the group consisting of:
(a) a nucleic acid molecule encoding a polypeptide shown in Table II, application no. 14, column 5 or 8, preferably shown in Table II B, application no. 14, column 8, or a homolog or a fragment thereof;
(b) a nucleic acid molecule shown in Table I, application no. 14, column 5 or 8, preferably shown in Table I B, application no. 14, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 14, preferably in column 8 of Table II B, application no. application no.;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 3, preferably shown in column 8 of Table I B, application no. 14, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 14;
(i) a nucleic acid molecule encoding a polypeptide having the activity represented by a respective protein as depicted in column 5 of Table II, application no. 14;
(j) a nucleic acid molecule comprising a polynucleotide which is obtained by amplifying a cDNA library or a genomic library using the primers in Table III, application no. 14, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
or a nucleic acid molecule comprising a sequence which is complementary thereto.

Item 7. A nucleic acid construct which confers the expression of the nucleic acid molecule as defined in item 6, comprising one or more regulatory elements.

Item 8. An expression cassette comprising
(a) a promoter, preferably selected from the group consisting of Big35S, PCUbi, Super and USP;
(b) a nucleic acid molecule as defined in item 6.

Item 9. A vector comprising the nucleic acid molecule as defined in item 6 or the nucleic acid construct as claimed in item 7 or the expression cassette as claimed in item 8.

Item 10. A host cell or a plant organelle, which has been transformed with the vector as claimed in item 9 or the nucleic acid molecule as defined in item 6 or the nucleic acid construct as claimed in item 7 or the expression cassette as claimed in item 8.

Item 11. The host cell of item 10, which is a plant cell or a microorganism.

Item 12. A process for producing a polypeptide, wherein the polypeptide is expressed in a host cell as claimed in item 10 or 11.

Item 13. A polypeptide produced by the process as claimed in item 12 or encoded by the nucleic acid molecule as defined in item 6.

Item 14. An antibody, which binds specifically to the polypeptide as claimed in item 13.

Item 15. A plant tissue, propagation material, harvested material or a plant comprising the host cell as claimed in item 10 or 11.

Item 16. A process for the identification of a compound conferring an increase in beta-sitosterol, campesterol and/or stigmasterol production in a non-human organism, comprising the steps:
(a) culturing a plant cell or tissue or microorganism or maintaining a plant expressing the polypeptide encoded by the nucleic acid molecule as defined in item 6 conferring an increase in the amount of beta-sitosterol, campesterol and/or stigmasterol in a non-human organism or a part thereof and a readout system capable of interacting with the polypeptide under suitable conditions which permit the interaction of the polypeptide with said readout system in the presence of a compound or a sample comprising a plurality of compounds and capable of providing a detectable signal in response to the binding of a compound to said polypeptide under conditions which permit the expression of said readout system and of the polypeptide encoded by the nucleic acid molecule of item 6 conferring an increase in the amount of beta-sitosterol, campesterol and/or stigmasterolin a non-human organism or a part thereof;
(b) identifying if the compound is an effective agonist by detecting the presence or absence or increase of a signal produced by said readout system.

Item 17. A method for the production of an agricultural composition comprising the steps of the method of item 16 and formulating the compound identified in item 16 in a form acceptable for an application in agriculture.

Item 18. A composition comprising the nucleic acid molecule as defined in item 6, the polypeptide as claimed in item 13, the nucleic acid construct as claimed in item 7, the expression cassette as claimed in item 8, the vector as claimed in item 9, the compound as claimed in item 16, the antibody as claimed in item 14, and optionally an agricultural acceptable carrier.

Item 19. Use of the nucleic acid molecule as defined in item 6 for the identification of a nucleic acid molecule conferring an increase in beta-sitosterol, campesterol and/or stigmasterol after expression.

Item 20. Cosmetic, pharmaceutical, food or feed composition comprising the nucleic acid moleculeas defined in item 6, the polypeptide as claimed in item 13, the nucleic acid construct as claimed in item 7, the expression cassette as claimed in item 8, the vector as claimed in item 9, the antibody as claimed in item 14 , the plant or plant tissue as claimed in item 15, the harvested material as claimed in item 15 or the host cell of item as claimed in items 10 or 11.

Item 21. Use of the nuclei acid molecule as defined in item 6, the polypeptide as claimed in item 13, the nucleic acid construct as claimed in item 7, the expression cassette as claimed in item 8, the vector as claimed in claim9, the plant or plant tissue as claimed in item 15, or the host cell of item 10 to 11 for the production of plant resistant to a herbicide inhibiting the production of beta-sitosterol, campesterol and/or stigmasterol.

Item 22. A method for producing a plant with increased yield as compared to a corresponding non-transformed wild type plant, whereby the method comprises
(i) increasing or generating of the expression of; and/or
(ii) increasing or generating the expression of an expression product of; and/or
(iii) increasing or generating one or more activities of an expression product encoded by;
   at least one respective nucleic acid molecule comprising a nucleic acid molecule selected from the group consisting of:
(a) a nucleic acid molecule encoding the polypeptide shown in column 5 or 8 of Table II, preferably Table II B, application no. 14, or a homolog or a fragment thereof, in case in column 7 beta-sitosterol, campesterol and/or stigmasterol is indicated;
(b) a nucleic acid molecule shown in column 5 or 8 of Table I, preferably Table I B, application no. 14, or a homolog or a fragment thereof (preferably the coding region thereof) , in case in column 7 beta-sitosterol, campesterol and/or stigmasterol is indicated,
(c) a nucleic acid molecule, which, as a result of the degeneracy of the genetic code, can be derived from a polypeptide sequence depicted in column 5 or 8 of Table II, preferably Table II B, application no. 14, in case in column 7 beta-sitosterol, campesterol and/or stigmasterol is indicated;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 14, or the coding region thereof, in case in column 7 beta-sitosterol, campesterol and/or stigmasterol is indicated;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 14, in case in column 7 beta-sitosterol, campesterol and/or stigmasterol is indicated;
(i) a nucleic acid molecule encoding a polypeptide having the activity represented by a respective protein as depicted in column 5 of Table II, application no. 14, in case in column 7 beta-sitosterol, campesterol and/or stigmasterol is indicated;
(j) a nucleic acid molecule which comprises a polynucleotide, which is obtained by amplifying a cDNA library or a genomic library using the primers as depicted in column 8 of Table III, application no. 14, in case in column 7 beta-sitosterol, campesterol and/or stigmasterol is indicated; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto; in a plant cell, plant or a part thereof.

23. A method for producing a plant with increased yield according to item 22, which comprises in addition the regeneration of a plant from such transformed plant cell, plant cell compartment, plant cell nucleus, plant cell or plant tissue with increased yield.

[0483.1.7.14] to [0492.1.7.14] for the disclosure of theses paragraphs see [0483.1.7.7] to [0492.1.7.7] above

### Example 10

### Purification of a Fine Chemical e.g. of Phytosterols in Particular Beta-Sitosterol, Campesterol, and/or Stigmasterol

Identity and purity of the phytosterols such as beta-sitosterol, campesterol, and/or stigmasterol isolated can be determined by standard techniques of the art. They encompass old techniques such as photometric methods and new techniques such as high-performance liquid chromatography (HPLC), gas chromatography (GC), spectroscopic methods, mass spectrometry (MS), staining methods, thin-layer chromatography, NIRS, enzyme assay or microbiological assays. Phytosterols can be detected via HPLC, e.g. reversed-phase HPLC, as described by Heftmann, E. and Hunter, I.R. (J Chromatogr 1979; 165: 283-299. Furthermore the analysis of phytosterols can be performed as described in S. L. Abidi, “Chromatographic analysis of plant sterols in foods and vegetable oils [Review]”, Journal of Chromatography 2001, A.. 935, 173-20 or P. Appelblad, K. Irgum, “Separation and detection of neuroactive steroids from biological matrices [Review]”, Journal of Chromatography A 2002, 955, 151-182. or K. Shimada, K. Mitamura, T. Higashi, “Gas chromatography and high-performance liquid chromatography of natural steroids [Review]”, Journal of Chromatography 2001, A.. 935, 141-172. For the sterol isolation and analysis the German standard method F III may be used. The method comprises: saponification of fat, isolation of unsaponifiable matter using an aluminium oxide column, separation of sterol fraction by preparative TLC and determination of the composition of sterols as trimetysilyl ethers by GC.

for the disclosure of this paragraph see [0494.1.7.7] above

### Example 11a

### PCR Amplification of the Sequences

Unless otherwise specified, standard methods as described in Sambrook et al., Molecular Cloning: A laboratory manual, Cold Spring Harbor 1989, Cold Spring Harbor Laboratory Press are used.

The inventive sequences as shown in the respective line in Table I, column 5, preferably the coding region thereof, were amplified by PCR as described in the protocol of the Pfu Ultra, Pfu Turbo or Herculase DNA polymerase (Stratagene). The composition for the protocol of the Pfu Ultra, Pfu Turbo or Herculase DNA polymerase was as follows: 1×PCR buffer (Stratagene), 0.2 mM of each dNTP, 100 ng genomic DNA of Saccharomyces cerevisiae (strain S288C; Research Genetics, Inc., now Invitrogen), Escherichia coli (strain MG1655; E. coli Genetic Stock Center), Synechocystis sp. (strain PCC6803), Azotobacter vinelandii (strain N.R. Smith, 16), Thermus thermophilus (HB8) or 50 ng cDNA from various tissues and development stages of Arabidopsis thaliana (ecotype Columbia), Physcomitrella patens, Glycine max (variety Resnick), Brassica napus, Oryza sativa or Zea mays (variety B73, Mo17, A188), 50 pmol forward primer, 50 pmol reverse primer, with or without 1 M Betaine, 2.5 u Pfu Ultra, Pfu Turbo or Herculase DNA polymerase.

The amplification cycles were as follows:

1 cycle of 2-3 minutes at 94-95° C., then 25-36 cycles with 30-60 seconds at 94-95° C., 30-45 seconds at 50-60° C. and 210-480 seconds at 72° C., followed by 1 cycle of 5-10 minutes at 72° C., then 4-16° C.—preferably for Saccharomyces cerevisiae, Escherichia coli, Synechocystis sp., Azotobacter vinelandii, Thermus thermophilus.

In case of Arabidopsis thaliana, Brassica napus, Glycine max, Oryza sativa, Physcomitrella patens, Zea mays the amplification cycles were as follows: 1 cycle with 30 seconds at 94° C., 30 seconds at 61° C., 15 minutes at 72° C., then 2 cycles with 30 seconds at 94° C., 30 seconds at 60° C., 15 minutes at 72° C., then 3 cycles with 30 seconds at 94° C., 30 seconds at 59° C., 15 minutes at 72° C., then 4 cycles with 30 seconds at 94° C., 30 seconds at 58° C., 15 minutes at 72° C., then 25 cycles with 30 seconds at 94° C., 30 seconds at 57° C., 15 minutes at 72° C., then 1 cycle with 10 minutes at 72° C., then finally 4-16° C. RNAs were generated with the RNeasy Plant Kit according to the standard protocol (Qiagen) and Superscript II Reverse Transkriptase was used to produce double stranded cDNA according to the standard protocol (Invitrogen).

ORF specific primer pairs for the genes to be expressed are shown in the respective line in Table III, column 8. The following adapter sequences were added to Saccharomyces cerevisiae ORF specific primers (see Table III) for cloning purposes:

SEQ ID NO: 20

| | | |
|---|---|---|
| i) | foward primer: | 5′-GGAATTCCAGCTGACCACC-3′ |

SEQ ID NO: 21

| | | |
|---|---|---|
| ii) | reverse primer: | 5′-GATCCCCGGGAATTGCCATG-3′ |

These adaptor sequences allow cloning of the ORF into the various vectors containing the Resgen adaptors, see table column E of Table c.

The following adapter sequences were added to Saccharomyces cerevisiae, Escherichia coli, Synechocystis sp., Azotobacter vinelandii, Thermus thermophilus, Arabidopsis thaliana, Brassica napus, Glycine max, Oryza sativa , Physcomitrella patens, or Zea mays ORF specific primers for cloning purposes:

| SEQ ID NO: 22 | | |
|---|---|---|
| iii) | forward primer: | 5′-TTGCTCTTCC-3′ |
| SEQ ID NO: 23 | | |
| iiii) | reverse primer: | 5′-TTGCTCTTCG-3′ |

The adaptor sequences allow cloning of the ORF into the various vectors containing the Colic adaptors, see table column E of Table c.

Therefore for amplification and cloning of Saccharomyces cerevisiae SEQ ID NO: 14171, a primer consisting of the adaptor sequence i) and the ORF specific sequence SEQ ID NO: 14271 and a second primer consisting of the adaptor sequence ii) and the ORF specific sequence SEQ ID NO: 14272 were used.

For amplification and cloning of Escherichia coli SEQ ID NO: 7333, a primer consisting of the adaptor sequence iii) and the ORF specific sequence SEQ ID NO: 7679 and a second primer consisting of the adaptor sequence iiii) and the ORF specific sequence SEQ ID NO: 7680 were used.

For amplification and cloning of Synechocystis sp. SEQ ID NO: 11990, a primer consisting of the adaptor sequence iii) and the ORF specific sequence SEQ ID NO: 12064 and a second primer consisting of the adaptor sequence iiii) and the ORF specific sequence SEQ ID NO: 12065 were used.

For amplification and cloning of Arabidopsis thaliana SEQ ID NO: 1061, a primer consisting of the adaptor sequence iii) and the ORF specific sequence SEQ ID NO: 1293 and a second primer consisting of the adaptor sequence iiii) and the ORF specific sequence SEQ ID NO: 1294 were used.

For amplification and cloning of Azotobacter vinelandii SEQ ID NO: 6075, a primer consisting of the adaptor sequence iii) and the ORF specific sequence SEQ ID NO: 6255 and a second primer consisting of the adaptor sequence iiii) and the ORF specific sequence SEQ ID NO: 6256 were used.

For amplification and cloning of Glycine max SEQ ID NO: 10811, a primer consisting of the adaptor sequence iii) and the ORF specific sequence SEQ ID NO: 11207 and a second primer consisting of the adaptor sequence iiii) and the ORF specific sequence SEQ ID NO: 11208 were used.

For amplification and cloning of Thermus thermophilus SEQ ID NO: 12974, a primer consisting of the adaptor sequence iii) and the ORF specific sequence SEQ ID NO: 13372 and a second primer consisting of the adaptor sequence iiii) and the ORF specific sequence SEQ ID NO: 13373 were used.

Following these examples every sequence disclosed in Table I, preferably column 5, especially the coding region thereof can be cloned by fusing the adaptor sequences to the respective specific primers sequences as disclosed in Table III, column 8 using the respective vectors shown in Table c.

[0496.1.7.14] to [0499.1.7.14] for the disclosure of these paragraphs see [0496.1.7.7] to [0499.1.7.7] above

### Example 11e

### Cloning of Inventive Sequences as Shown in Table I, Column 5 in the Different Expression Vectors

For cloning of for example SEQ ID NO: 7333 from Escherichia coli or any other ORF from Escherichia coli, Synechocystis sp., Azotobacter vinelandii, Thermus thermophilus, Arabidopsis thaliana or Glycine max, the vector DNA was treated with the restriction enzymes Pacl and Ncol following the standard protocol (MBI Fermentas).

For cloning for example the ORFs of SEQ ID NO: 14171 from Saccharomyces cerevisiae or any other ORF from Saccharomyces cerevisiae into vectors containing the Resgen adaptor sequence the respective vector DNA was treated with the restriction enzyme Ncol.

For cloning of for example ORFs SEQ ID NO: 66274 from Saccharomyces cerevisiae or any other ORF from Saccharomyces cerevisiae into vectors containing the Colic adaptor sequence, the respective vector DNA was treated with the restriction enzymes Pacl and Ncol following the standard protocol (MBI Fermentas).

In all cases the reaction was stopped by inactivation at 70° C. for 20 minutes and purified over QlAquick or NucleoSpin Extract II columns following the standard protocol (Qiagen or Macherey-Nagel).

Then the PCR-product representing the amplified ORF with the respective adapter sequences and the vector DNA were treated with T4 DNA polymerase according to the standard protocol (MBI Fermentas) to produce single stranded overhangs with the parameters 1 unit T4 DNA polymerase at 37° C. for 2-10 minutes for the vector and 1-2 u T4 DNA polymerase at 15-17° C. for 10-60 minutes for the PCR product representing SEQ ID NO: 7333.

The reaction was stopped by addition of high-salt buffer and purified over QlAquick or NucleoSpin Extract II columns following the standard protocol (Qiagen or Macherey-Nagel).

According to this example the skilled person is able to clone all sequences disclosed in Table I, preferably column 5 or column 8, especially the coding region thereof.

Approximately 30-60 ng of prepared vector and a defined amount of prepared amplificate were mixed and hybridized at 65° C. for 15 minutes followed by 37° C. 0.1° C/1 seconds, followed by 37° C. 10 minutes, followed by 0.1° C/1 seconds, then 4-10° C. The ligated constructs were transformed in the same reaction vessel by addition of competent E. coli cells (strain DHSalpha) and incubation for 20 minutes at 1° C. followed by a heat shock for 90 seconds at 42° C. and cooling to 1-4° C. Then, complete medium (SOC) was added and the mixture was incubated for 45 minutes at 37° C. The entire mixture was subsequently plated onto an agar plate with 0.05 mg/ml kanamycin and incubated overnight at 37° C.

The outcome of the cloning step was verified by amplification with the aid of primers which bind upstream and downstream of the integration site, thus allowing the amplification of the insertion. The amplifications were carried out as described in the protocol of Taq DNA polymerase (Gibco-BRL).

The amplification cycles were as follows:

1 cycle of 1-5 minutes at 94° C., followed by 35 cycles of in each case 15-60 seconds at 94° C., 15-60 seconds at 50-66° C. and 5-15 minutes at 72° C., followed by 1 cycle of 10 minutes at 72° C., then 4-16° C.

Several colonies were checked, but only one colony for which a PCR product of the expected size was detected was used in the following steps.

A portion of this positive colony was transferred into a reaction vessel filled with complete medium (LB) supplemented with kanamycin and incubated overnight at 37° C.

The plasmid preparation was carried out as specified in the Qiaprep or NucleoSpin Multi-96 Plus standard protocol (Qiagen or Macherey-Nagel).

[0501.1.7.14] to [0503.1.7.14] for the disclosure of these paragraphs see [0501.1.7.7] to [0503.1.7.7] above

Table d showing results of plant analyses

TABLE d

| SeqID | Target | Locus | Metabolite | Source | Promotor | Method | Min | Max |
|---|---|---|---|---|---|---|---|---|
| 131003 | non-targ | At1g07420 | campesterol | ARA_LEAF | p-PcUBI | GC | 89 | 300 |
| 17451 | non-targ | At1g07430 | campesterol | ARA_LEAF | p-PcUBI | GC | 18 | 54 |
| 17968 | non-targ | At1g36730 | campesterol | ARA_LEAF | p-PcUBI | GC | 17 | 36 |
| 18869 | non-targ | At1g61950 | b-sitosterol | ARA_LEAF | p-PcUBI | GC | 17 | 22 |
| 18869 | non-targ | At1g61950 | campesterol | ARA_LEAF | p-PcUBI | GC | 17 | 37 |
| 1061 | non-targ | At1g68320 | b-sitosterol | ARA_LEAF | p-PcUBI | GC | 14 | 49 |
| 1061 | non-targ | At1g68320 | campesterol | ARA_LEAF | p-PcUBI | GC | 16 | 76 |
| 19419 | non-targ | At1g72770 | campesterol | ARA_LEAF | p-PcUBI | GC | 17 | 79 |
| 21902 | non-targ | At3g04710 | campesterol | ARA_LEAF | p-PcUBI | GC | 16 | 31 |
| 109717 | non-targ | At4g09960 | b-sitosterol | ARA_LEAF | p-PcUBI | GC | 17 | 154 |
| 109717 | non-targ | At4g09960 | campesterol | ARA_LEAF | p-PcUBI | GC | 17 | 38 |
| 131208 | non-targ | At4g12110 | campesterol | ARA_LEAF | p-PcUBI | GC | 22 | 75 |
| 23482 | non-targ | At4g15660 | b-sitosterol | ARA_LEAF | p-PcUBI | GC | 15 | 44 |
| 68849 | non-targ | At4g26080 | campesterol | ARA_LEAF | p-PcUBI | GC | 16 | 39 |
| 4348 | non-targ | At4g35310 | b-sitosterol | ARA_LEAF | p-PcUBI | GC | 18 | 34 |
| 4348 | non-targ | At4g35310 | campesterol | ARA_LEAF | p-PcUBI | GC | 35 | 70 |
| 5318 | non-targ | At5g57050 | campesterol | ARA_LEAF | p-PcUBI | GC | 30 | 45 |
| 25498 | non-targ | At5g59220 | b-sitosterol | ARA_LEAF | p-PcUBI | GC | 13 | 39 |
| 122803 | non-targ | At5g66710 | b-sitosterol | ARA_LEAF | p-PcUBI | GC | 14 | 23 |
| 26196 | non-targ | Avin- | b-sitosterol | ARA_LEAF | p-PcUBI | GC | 13 | 18 |
| | | DRAFT_1624 | | | | | | |
| 6075 | non-targ | Avin- | campesterol | ARA_LEAF | p-PcUBI | GC | 18 | 43 |
| | | DRAFT_3028 | | | | | | |
| 29500 | non-targ | Avin- | campesterol | ARA_LEAF | p-PcUBI | GC | 16 | 35 |
| | | DRAFT_3253 | | | | | | |
| 31026 | non-targ | Avin- | b-sitosterol | ARA_LEAF | p-PcUBI | GC | 13 | 24 |
| | | DRAFT_3605 | | | | | | |
| 31026 | non-targ | Avin- | campesterol | ARA_LEAF | p-PcUBI | GC | 17 | 44 |
| | | DRAFT_3605 | | | | | | |
| 114231 | non-targ | Avin- | campesterol | ARA_LEAF | p-PcUBI | GC | 16 | 72 |
| | | DRAFT_3629 | | | | | | |
| 6510 | non-targ | Avin- | b-sitosterol | ARA_LEAF | p-PcUBI | GC | 16 | 32 |
| | | DRAFT_5103 | | | | | | |
| 6510 | non-targ | Avin- | campesterol | ARA_LEAF | p-PcUBI | GC | 19 | 45 |
| | | DRAFT_5103 | | | | | | |
| 34889 | plastidic | B0004 | campesterol | ARA_LEAF | p-Super | GC | 21 | 90 |
| 35204 | non-targ | B0061 | campesterol | ARA_LEAF | p-Super | GC | 16 | 56 |
| 35482 | non-targ | B0124 | campesterol | ARA_SEED_2 | p-USP | GC | 26 | 55 |
| 7333 | non-targ | B0449 | b-sitosterol | ARA_LEAF | p-Super | GC | 19 | 63 |
| 7333 | non-targ | B0449 | campesterol | ARA_LEAF | p-Super | GC | 20 | 86 |
| 7686 | non-targ | B0486 | campesterol | ARA_LEAF | p-Super | GC | 19 | 29 |
| 131359 | plastidic | B0856 | campesterol | ARA_LEAF | p-Super | GC | 25 | 28 |
| 76032 | plastidic | B1065 | b-sitosterol | ARA_LEAF | p-Super | GC | 20 | 27 |
| 37400 | non-targ | B1186 | b-sitosterol | ARA_LEAF | p-Super | GC | 15 | 54 |
| 37400 | non-targ | B1186 | campesterol | ARA_LEAF | p-Super | GC | 16 | 39 |
| 131489 | non-targ | B1391 | b-sitosterol | ARA_LEAF | p-Super | GC | 13 | 24 |
| 131489 | non-targ | B1391 | campesterol | ARA_LEAF | p-Super | GC | 15 | 29 |
| 131531 | non-targ | B1401 | campesterol | ARA_LEAF | p-Super | GC | 19 | 40 |
| 38266 | plastidic | B1431 | campesterol | ARA_LEAF | p-Super | GC | 27 | 440 |
| 38300 | non-targ | B1597 | b-sitosterol | ARA_LEAF | p-Super | GC | 20 | 33 |
| 38300 | non-targ | B1597 | campesterol | ARA_LEAF | p-Super | GC | 16 | 46 |
| 131539 | non-targ | B1795 | campesterol | ARA_LEAF | p-Super | GC | 37 | 54 |
| 131799 | non-targ | B1932 | campesterol | ARA_LEAF | p-Super | GC | 17 | 50 |
| 39002 | non-targ | B2107 | campesterol | ARA_LEAF | p-Super | GC | 16 | 48 |
| 131839 | non-targ | B2276 | campesterol | ARA_SEED_2 | p-USP | GC | 13 | 20 |
| 132086 | non-targ | B2312 | campesterol | ARA_LEAF | p-Super | GC | 16 | 73 |
| 132695 | plastidic | B2376 | campesterol | ARA_LEAF | p-Super | GC | 26 | 193 |
| 39237 | non-targ | B2399 | campesterol | ARA_LEAF | p-Super | GC | 45 | 79 |
| 40329 | non-targ | B2474 | campesterol | ARA_LEAF | p-Super | GC | 17 | 48 |
| 132706 | non-targ | B2493 | campesterol | ARA_LEAF | p-Super | GC | 16 | 29 |
| 9167 | non-targ | B2513 | campesterol | ARA_LEAF | p-Super | GC | 37 | 72 |
| 99898 | non-targ | B2546 | b-sitosterol | ARA_LEAF | p-Super | GC | 13 | 21 |
| 99898 | non-targ | B2546 | campesterol | ARA_LEAF | p-Super | GC | 16 | 30 |
| 125226 | non-targ | B2654 | campesterol | ARA_LEAF | p-Super | GC | 14 | 21 |
| 80756 | non-targ | B2701 | campesterol | ARA_LEAF | p-Super | GC | 16 | 37 |
| 132821 | plastidic | B2766 | campesterol | ARA_LEAF | p-Super | GC | 20 | 39 |
| 115864 | non-targ | B2866 | campesterol | ARA_LEAF | p-Super | GC | 30 | 32 |
| 42477 | non-targ | B3151 | campesterol | ARA_LEAF | p-Super | GC | 19 | 46 |
| 42502 | non-targ | B3262 | b-sitosterol | ARA_LEAF | p-Super | GC | 12 | 26 |
| 42600 | non-targ | B3616 | campesterol | ARA_LEAF | p-Super | GC | 19 | 24 |
| 44372 | non-targ | B3989 | b-sitosterol | ARA_LEAF | p-Super | GC | 12 | 27 |
| 44372 | non-targ | B3989 | campesterol | ARA_LEAF | p-Super | GC | 37 | 49 |
| 10740 | non-targ | B4029 | b-sitosterol | ARA_LEAF | p-Super | GC | 19 | 78 |
| 10740 | non-targ | B4029 | stigmasterol | ARA_LEAF | p-Super | GC | 80 | 3076 |
| 126975 | plastidic | B4205 | b-sitosterol | ARA_LEAF | p-Super | GC | 13 | 16 |
| 10811 | non-targ | GM02LC12622 | b-sitosterol | ARA_LEAF | p-PcUBI | GC | 12 | 37 |
| 10811 | non-targ | GM02LC12622 | campesterol | ARA_LEAF | p-PcUBI | GC | 16 | 36 |
| 46751 | non-targ | GM02LC19289 | b-sitosterol | ARA_LEAF | p-PcUBI | GC | 14 | 40 |
| 46751 | non-targ | GM02LC19289 | campesterol | ARA_LEAF | p-PcUBI | GC | 17 | 52 |
| 46850 | non-targ | GM02LC44512 | b-sitosterol | ARA_LEAF | p-PcUBI | GC | 13 | 30 |
| 46850 | non-targ | GM02LC44512 | campesterol | ARA_LEAF | p-PcUBI | GC | 20 | 56 |
| 127103 | non-targ | Sll0406 | campesterol | ARA_LEAF | p-PcUBI | GC | 16 | 32 |
| 132874 | non-targ | Sll0671 | campesterol | ARA_LEAF | p-PcUBI | GC | 16 | 43 |
| 53878 | mitochondrial | Sll1443 | b-sitosterol | ARA_LEAF | p-PcUBI | GC | 12 | 25 |
| 53878 | mitochondrial | Sll1443 | campesterol | ARA_LEAF | p-PcUBI | GC | 17 | 21 |
| 132938 | mitochondrial | Sll1525 | b-sitosterol | ARA_LEAF | p-PcUBI | GC | 17 | 24 |
| 85743 | mitochondrial | Sll1598 | b-sitosterol | ARA_LEAF | p-PcUBI | GC | 17 | 20 |
| 55379 | non-targ | Sll1761 | campesterol | ARA_LEAF | p-PcUBI | GC | 22 | 44 |
| 86447 | plastidic | Sll1848 | b-sitosterol | ARA_LEAF | p-PcUBI | GC | 16 | 26 |
| 55385 | mitochondrial | Sll1920 | campesterol | ARA_LEAF | p-PcUBI | GC | 20 | 66 |
| 11990 | non-targ | Slr0338 | b-sitosterol | ARA_LEAF | p-PcUBI | GC | 14 | 19 |
| 119222 | mitochondrial | Slr0949 | campesterol | ARA_LEAF | p-PcUBI | GC | 16 | 28 |
| 87655 | plastidic | Slr0966 | campesterol | ARA_LEAF | p-PcUBI | GC | 18 | 20 |
| 61723 | non-targ | TTC1193 | b-sitosterol | ARA_LEAF | p-PcUBI | GC | 15 | 17 |
| 61723 | non-targ | TTC1193 | campesterol | ARA_LEAF | p-PcUBI | GC | 17 | 39 |
| 62079 | non-targ | TTC1386 | b-sitosterol | ARA_LEAF | p-PcUBI | GC | 24 | 31 |
| 62079 | non-targ | TTC1386 | campesterol | ARA_LEAF | p-PcUBI | GC | 34 | 50 |
| 12974 | non-targ | TTC1550 | campesterol | ARA_LEAF | p-PcUBI | GC | 17 | 23 |
| 113224 | plastidic | Ydl131w | campesterol | ARA_LEAF | p-Super | GC | 18 | 98 |
| 14171 | non-targ | Ydl155w | campesterol | ARA_LEAF | Big35S | GC | 17 | 63 |
| 113313 | plastidic | Ydl235c | campesterol | ARA_LEAF | p-Super | GC | 21 | 37 |
| 120436 | plastidic | Ygl039w | campesterol | ARA_LEAF | p-PcUBI | GC | 17 | 33 |
| 66274 | non-targ | Yjr153w | campesterol | ARA_LEAF | p-PcUBI | GC | 16 | 42 |
| 129546 | plastidic | Ynl321w | campesterol | ARA_LEAF | p-Super | GC | 16 | 28 |
| 92668 | non-targ | Ypl028w | b-sitosterol | ARA_SEED_2 | p-PcUBI | GC | 12 | 23 |
| 113647 | plastidic | Ypr170c | b-sitosterol | ARA_LEAF | p-Super | GC | 15 | 32 |
| 16883 | plastidic | 59582753_SOYBEAN | campesterol | ARA_LEAF | p-PcUBI | GC | 18 | 52 |

Column 1 shows the SEQ ID NO, Column 2 shows the expression type (targeted or non-targeted), Column 3 shows the “gene name” (locus), Column 4 shows the metabolite analyzed, Column 5 indicates the A. thaliana source tissue analyzed, Column 6 indicates the used promoter for expression, Column 7 indicates the analytical method. Columns 8 and 9 show the minimum and the maximum increase of the analyzed metabolite (in percent) in comparison to the wild type (ratio_by_WT, given as percent increase).

The term “non-targ” in Table d, Column 2 which shows the expression type means “non-targeted”.

The term “b-sitosterol” in Table d, Column 4 which shows the metabolite means “beta-sitosterol”. When the analyses were repeated independently, all results proved to be significant.

[0505.1.7.14] to [0515.1.7.14] for the disclosure of these paragraphs see [0505.1.7.7] to [0515.1.7.7] above

In a further embodiment, the present invention relates in paragraphs [0000.1.7.15.] to [0514.1.7.15.] to a further process for the production of the fine chemical citramalate, citrate, fumarate, glycerate, malate, oxalate, pyruvate, succinate, and/or vitamin c and/or threonic acid as defined below and corresponding embodiments as described herein as follows.

for the disclosure of this paragraph see [0001.1.7.7] above

Succinic acid (succinate) is a dicarboxylic acid, a water-soluble, colorless crystal with an acid taste. It is an intermediate of the citric acid cycle (known also as the tricarboxylic acid cycle (TCA cycle) or the Krebs cycle) and the glyoxylate cycle, produced by action of the enzyme succinyl-CoA synthetase on succinyl-CoA. Succinic acid is converted to fumaric acid by action of the enzyme succinic acid dehydrogenase (with formation of FADH2).

It is used as flavoring agent for food and beverages. Succinic acid is an intermediate for a lot of chemical compounds such as dyes, perfumes, lacquers, photographic chemicals, alkyd resins or plasticizer. Furthermore it is also an intermediate used for the production of medicines used as sedatives, contraceptives or anticancer drugs.

Malic acid (malate) is an alpha-hydroxy organic acid. Its salt is named as malate. It is found in apples and other fruits and therefore named as fruit acid. Malic acid especially in the form of its anion malate, is a key intermediate in the major biochemical energyproducing cycle in cells known as the citric acid or Krebs cycle located in the cells' mitochondria and in the glyoxylate cycle.

It is an important compound together with magnesium for the treatment of fibromyalgia, a rheumatic illness which affects often middle-aged women. D-Malate is an optically active compound which can be used as a synthon in organic synthesis, as a resolving agent and as a ligand in asymmetric catalysis.

Malic acid is not only a central metabolite in intermediary flow of carbon in organisms. Malic acid is a common constituent of all plants, and its formation is controlled by an enzyme (protein catalyst) called malic acid dehydrogenase (MDH).

In higher plants, vacuolar malic acid accumulation, and hence, transtonoplast malic acid transport, also plays a paramount role in many physiological functions. These include adjustment of osmotic and turgor potentials in extension growth and movements of stomata and pulvini, pHregulation, e.g. during nitrate reduction, and others (for review, see Lunge et al, Plant Physiol, 124(2000),1335-1348).

Malic acid occupies a central role in plant metabolism. Its importance in plant mineral nutrition is reflected by the role it plays in symbiotic nitrogen fixation, phosphorus acquisition, and aluminum tolerance (Osawa and Matsumoto, Plant Physiol, 126(2001), 411-420). During phosphorus deficiency, malic acid is frequently secreted from roots to release unavailable forms of phosphorus. In nitrogen-fixing root nodules, malic acid is the primary substrate for bacteroid respiration, thus fueling nitrogenase. The malic acid oxaloacetate shuttle is characteristic for plant cells. It transports redox equivalents intracellularly.

It should be noted that malate (and pyruvate as phosphoenolpyruvate) have a special function as intermediate storage form for carbon dioxide in the C4 and CAM metabolism.

Ascorbic acid (Vitamin C, ascorbate) is a six carbon compound related to glucose, is a water-soluble vitamin that is naturally present in some foods, added to others, and available as a dietary supplement. It is found naturally in citrus fruits and many vegetables. Ascorbic acid is an essential nutrient in human diets, and necessary to maintain connective tissue and bone. Its biologically active form, vitamin C, functions as a reducing agent and coenzyme in several metabolic pathways. Vitamin C is considered an antioxidant. Its appearance is white to light yellow crystals or powder. It is water soluble. The L-enantiomer of ascorbic acid is commonly known as vitamin C. Ascorbic acid also rapidly interconverts into two unstable diketone tautomers by proton transfer, although it is the most stable in the enol form. Primates (including humans) and a few other species in all divisions of the animal kingdom, notably the guinea pig, are unable to synthesize vitamin C endogenously, so it is an essential dietary component.

The metabolism of ascorbic acid involves its oxidation to dehydroascorbic acid, followed by the formation of the aldonic acids L-lyxonic acid, L-xylonic acid, and L-threonic acid.

Threonic acid is a sugar acid derived from threose. The L-isomer is a metabolite of ascorbic acid (vitamin C).Threonic acid is a natural constituent in leaves of certain plants. It is derived from ascorbate either by enzymatic or natural oxidation.

Fumaric acid (fumarate) is also an intermediate of the citric acid cycle (Krebs cycle). It is synthesized from succinic acid. Fumarate, also called fumaric acid, is a useful compound in treatment of psoriasis, which is a chronic, incurable, disabling skin disease characterised by red, scaly plaques. Approximately 23% of psoriasis patients also have an accompanying arthritis that can become debilitating.

The discovery in Arabidopsis of citramalic acid (citramalate) (Fiehn et al. 2000 Nature Biotechnology 18, 1157-1161) a potential precursor of pyruvic acid and acetate suggests a novel aspect of carbon metabolism and furthermore suggests the existence of a tricarboxylic acid cycle bypass previously found only in bacteria. Citramalate is an derivative of malic acid and produced from itaconic acid.

Glyceric acid (glycerate) is an important precursor in the anabolism of amino acids, in particular for serine and glycine. Following the glucollate pathway glyceric acid can be converted into pyruvate. Further, the energy level of a cell may be depend on the level of glyceric acid found. Glycerate and glycerate-3-phophate form a shuttle for the transportation of energy equivalents, e.g. during photorespiration between glycosomes and peroxisomes. Glyceric acid is colorless syrupy acid, obtained from oxidation of glycerol and has an diuretic effect. Glyceric acid has an diuretic effect.

Citrate is the conjugated base of citric acid. Since citric acid is a multifunctional acid, intermediate ions exist (hydrogen citrate ion and dihydrogen citrate ion). The exact state of protonation/deprotonation of the citrate anion/citric acid is not discriminated here. Citrate is a key intermediate in the tricarboxylic acid cycle (TCA cycle) , which is of central importance for aerobic organisms and is a key intermediate in the glyoxylate cycle. Citrate as a metabolite has been observed in all kown living organisms .

Citrate from the TCA cylce can be branched of into fatty acid biosynthesis (via conversion to acetyl CoA). Citrate is a positive modulator of this conversion and allosterically regulates the enzyme acetyl-CoA carboxylase, which is the regulating enzyme in the conversion of acetyl CoA into malonyl CoA (the commitment step in fatty acid synthesis).

Pyruvic acid (pyruvates) is a naturally occurring component in plants and vegetables and in the body, where it is inherently involved in metabolism, the process whereby energy is produced. Pyruvate supplies energy to living cells in the citric acid cycle and can also be converted to carbohydrates via gluconeogenesis, to fatty acids or energy through acetylCoA, to the amino acid alanine and to ethanol. Pyruvic acid represents the final step in the metabolism of glucose or starch. Pyruvate can be derived from glyceric acid. Increased pyruvic acid production in yeast strains is known (WO 04/099425).

Pyruvic acid is used commercially to produce its salts and esters (pyruvates) used as dietary supplements for the effect of enhancing weight loss. Pyruvic acid is used for the synthesis of amino acids (alanine, tyrosine, phenylalanine, and tryptophan) and used in biochemical research. Its derivatives are used in making food additives and flavoring agents.

Oxalic acid is the simplest dicarboxylic acid. Reactions of protonisation/deprotonisation belong to the fastest chemical reactions known and it usually makes little sense to distinguish between the acid and it's conjugated base in the complex biological enviroments. Therefore, e.g. oxalic acid and oxalate are not commonly distinguished in this document—with the noteable exception of insoluble salts (e.g. calcium oxalate). Oxalic acid is formed via several major pathways. In plants, glycolate can be converted to oxalic acid. Oxaloacetate also can be split to form oxalate and acetate. Another significant precursor of oxalate in plants is L-ascorbic acid. The cleavage of ascorbic acid between C2 and C3 leads to oxalate biosynthesis.

Oxalic acid is heavily used in electrochemistry .Oxalic acid for technical applications is produced from natrium formiate or by fermentation of Aspergillus strains. Oxalic acid is considered to be relevant for plant health; nevertheless, the (minor) toxicity of solutable oxalates should be kept in mind when an increased oxalic acid content is desired for improved plant health.

Due to these important physiological roles and agrobiotechnological potential of citramalate, citrate, fumarate, glycerate, malate, oxalate, pyruvate, succinate, and/or vitamin c and/or threonic acid there is a need to identify the genes of enzymes and other proteins involved in citramalate, citrate, fumarate, glycerate, malate, oxalate, pyruvate, succinate, and/or vitamin c and/or threonic acid metabolism, and to generate mutants or transgenic non-human organism or a part thereof, preferably a microorganism, a plant cell, a plant or a part thereof, which are able to modify the content in said organism.

Improving the quality of foodstuffs and animal feeds is an important task of the food-and-feed industry. It is therefore a task to increase the productivity of plants so that the content of citramalate, citrate, fumarate, glycerate, malate, oxalate, pyruvate, succinate, and/or vitamin c and/or threonic acid in the plants are increased. Said products can be isolated from the plants and used for the production of cosmetics and pharmaceuticals.

On the other hand said fine chemicals citramalate, citrate, fumarate, glycerate, malate, oxalate, pyruvate, succinate, and/or vitamin c and/or threonic acid, are used also in industrial production and processes which go beyond production of food, feed, cosmetics and pharmaceuticals.

It is an object of the present invention to develop an inexpensive process for the synthesis of citramalate, citrate, fumarate, glycerate, malate, oxalate, pyruvate, succinate, and/or vitamin c and/or threonic acid.

It is a further object of the present invention to develop an inexpensive process for the synthesis of citramalate, citrate, fumarate, glycerate, malate, oxalate, pyruvate, succinate, and/or vitamin c and/or threonic acid and to assure that the citramalate, citrate, fumarate, glycerate, malate, oxalate, pyruvate, succinate, and/or vitamin c and/or threonic acid are more accessible and facilely to isolate and recover in an industrial scale from the producing organism, preferably from a plant.

for the disclosure of this paragraph see [0013.1.7.7] above

Accordingly, in a first embodiment, the invention relates to a process for the production of at least one fine chemical selected from the group consisting of: citramalate, citrate, fumarate, glycerate, malate, oxalate, pyruvate, succinate, and vitamin c and/or threonic acid, or, in other words, of the “fine chemical” or “fine chemical of the invention”.

The terms “fine chemical of the invention”, “fine chemical” or “the fine chemical” are used herein equally and relate in context of the paragraphs or sections [0014.1.15.15] to [0514.1.7.15] essentially to the metabolite or the metabolites indicated in column 7, application no. 15 of Tables I, II and IV in the respective line.

Further, the term “in context of any of the paragraphs [0014.1.15.15] to [0514.1.7.15]” as used herein means that for any of said paragraphs [0014.1.15.15] to [0514.1.7.15] the term “the fine chemical” is understood to follow the definition of paragraphs or sections [0014.1.15.15] and [0015.1.15.15], independently whether it refers to any other paragraph or not and whether the reference recites the term “fine chemical” in an other context.

Thus, in cases where one or more paragraphs or sections are incorporated by reference into any of the present paragraphs [0014.1.15.15] to [0514.1.7.15], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to the definition of paragraph [0014.1.15.15] and [0015.1.15.15].

Accordingly, in one embodiment, the term “fine chemical of the invention”, “fine chemical” or “the fine chemical” as used herein (alone or in combination, like in FCRP) means “citramalate in context of the nucleic acid or polypeptide sequences listed in the respective same line of any one of Tables I to IV of application no. 15 and indicating in column 7 the metabolite “citramalate”.

In one embodiment, the term citramalate or the term “fine chemical” mean in context of the paragraphs or sections [0014.1.15.15] to [0514.1.7.15] at least one chemical compound with an activity of the above mentioned citramalate, respectively.

Accordingly, in one embodiment, the term “fine chemical of the invention”, “fine chemical” or “the fine chemical” as used herein (alone or in combination, like in FCRP) means “citrate in context of the nucleic acid or polypeptide sequences listed in the respective same line of any one of Tables I to IV of application no. 15 and indicating in column 7 the metabolite “citrate”.

In one embodiment, the term citrate or the term “fine chemical” mean in context of the paragraphs or sections [0014.1.15.15] to [0514.1.7.15] at least one chemical compound with an activity of the above mentioned citrate, respectively.

Accordingly, in one embodiment, the term “fine chemical of the invention”, “fine chemical” or “the fine chemical” as used herein (alone or in combination, like in FCRP) means “fumarate in context of the nucleic acid or polypeptide sequences listed in the respective same line of any one of Tables I to IV of application no. 15 and indicating in column 7 the metabolite “fumarate”.

In one embodiment, the term fumarate or the term “fine chemical” mean in context of the paragraphs or sections [0014.1.15.15] to [0514.1.7.15] at least one chemical compound with an activity of the above mentioned fumarate, respectively.

Accordingly, in one embodiment, the term “fine chemical of the invention”, “fine chemical” or “the fine chemical” as used herein (alone or in combination, like in FCRP) means “glycerate in context of the nucleic acid or polypeptide sequences listed in the respective same line of any one of Tables I to IV of application no. 15 and indicating in column 7 the metabolite “glycerate”.

In one embodiment, the term glycerate or the term “fine chemical” mean in context of the paragraphs or sections [0014.1.15.15] to [0514.1.7.15] at least one chemical compound with an activity of the above mentioned glycerate, respectively.

Accordingly, in one embodiment, the term “fine chemical of the invention”, “fine chemical” or “the fine chemical” as used herein (alone or in combination, like in FCRP) means “malate in context of the nucleic acid or polypeptide sequences listed in the respective same line of any one of Tables I to IV of application no. 15 and indicating in column 7 the metabolite “malate”.

In one embodiment, the term malate or the term “fine chemical” mean in context of the paragraphs or sections [0014.1.15.15] to [0514.1.7.15] at least one chemical compound with an activity of the above mentioned malate, respectively.

Accordingly, in one embodiment, the term “fine chemical of the invention”, “fine chemical” or “the fine chemical” as used herein (alone or in combination, like in FCRP) means “oxalate in context of the nucleic acid or polypeptide sequences listed in the respective same line of any one of Tables I to IV of application no. 15 and indicating in column 7 the metabolite “oxalate”.

In one embodiment, the term oxalate or the term “fine chemical” mean in context of the paragraphs or sections [0014.1.15.15] to [0514.1.7.15] at least one chemical compound with an activity of the above mentioned oxalate, respectively.

Accordingly, in one embodiment, the term “fine chemical of the invention”, “fine chemical” or “the fine chemical” as used herein (alone or in combination, like in FCRP) means “pyruvate in context of the nucleic acid or polypeptide sequences listed in the respective same line of any one of Tables I to IV of application no. 15 and indicating in column 7 the metabolite “pyruvate”.

In one embodiment, the term pyruvate or the term “fine chemical” mean in context of the paragraphs or sections [0014.1.15.15] to [0514.1.7.15] at least one chemical compound with an activity of the above mentioned pyruvate, respectively.

Accordingly, in one embodiment, the term “fine chemical of the invention”, “fine chemical” or “the fine chemical” as used herein (alone or in combination, like in FCRP) means “succinate in context of the nucleic acid or polypeptide sequences listed in the respective same line of any one of Tables I to IV of application no. 15 and indicating in column 7 the metabolite “succinate”.

In one embodiment, the term succinate or the term “fine chemical” mean in context of the paragraphs or sections [0014.1.15.15] to [0514.1.7.15] at least one chemical compound with an activity of the above mentioned succinate, respectively.

Accordingly, in one embodiment, the term “fine chemical of the invention”, “fine chemical” or “the fine chemical” as used herein (alone or in combination, like in FCRP) means “vitamin c and/or threonic acid” in context of the nucleic acid or polypeptide sequences listed in the respective same line of any one of Tables I to IV of application no. 15 and indicating in column 7 the metabolite “vitamin c and/or threonic acid”.

In one embodiment, the term vitamin c and/or threonic acid or the term “fine chemical” mean in context of the paragraphs or sections [0014.1.15.15] to [0514.1.7.15] at least one chemical compound with an activity of the above mentioned vitamin c and/or threonic acid, respectively. [0015.1.15.15] Accordingly, in one embodiment, the terms “fine chemical of the invention”, “fine chemical” or “the fine chemical” mean in context of any of the paragraphs [0014.1.15.15] to [0514.1.7.15] citramalate, its salts, ester or ether in free form or bound form. In a preferred embodiment, the term “the fine chemical” means citramalate or its salts, ester or ether, in free form or bound form.

In a preferred embodiment “citramalate” means citramalate in free form.

Accordingly, in one embodiment, the terms “fine chemical of the invention”, “fine chemical” or “the fine chemical” mean in context of any of the paragraphs [0014.1.15.15] to [0514.1.7.15] citrate, its salts, ester or ether in free form or bound form. In a preferred embodiment, the term “the fine chemical” means citrate or its salts, ester or ether, in free form or bound form.

In a preferred embodiment “citrate” means citrate in free form.

Accordingly, in one embodiment, the terms “fine chemical of the invention”, “fine chemical” or “the fine chemical” mean in context of any of the paragraphs [0014.1.15.15] to [0514.1.7.15] fumarate, its salts, ester or ether in free form or bound form. In a preferred embodiment, the term “the fine chemical” means fumarate or its salts, ester or ether, in free form or bound form.

In a preferred embodiment “fumarate” means fumarate in free form.

Accordingly, in one embodiment, the terms “fine chemical of the invention”, “fine chemical” or “the fine chemical” mean in context of any of the paragraphs [0014.1.15.15] to [0514.1.7.15] glycerate, its salts, ester or ether in free form or bound form. In a preferred embodiment, the term “the fine chemical” means glycerate or its salts, ester or ether, in free form or bound form, preferably as phosphate derivatives of glyceric acid, including 2-phosphoglyceric acid, 3-phosphoglyceric acid, 2,3-bisphosphoglyceric acid, and 1,3-bisphosphoglyceric acid.

In a preferred embodiment “glycerate” means glycerate in free form.

Accordingly, in one embodiment, the terms “fine chemical of the invention”, “fine chemical” or “the fine chemical” mean in context of any of the paragraphs [0014.1.15.15] to [0514.1.7.15] malate, its salts, ester or ether in free form or bound form. In a preferred embodiment, the term “the fine chemical” means malate or its salts, ester or ether, in free form or bound form.

In a preferred embodiment “malate” means malate in free form.

Accordingly, in one embodiment, the terms “fine chemical of the invention”, “fine chemical” or “the fine chemical” mean in context of any of the paragraphs [0014.1.15.15] to [0514.1.7.15] oxalate, its salts, ester or ether in free form or bound form. In a preferred embodiment, the term “the fine chemical” means oxalate or its salts, ester or ether, in free form or bound form.

In a preferred embodiment “oxalate” means oxalate in free form.

Accordingly, in one embodiment, the terms “fine chemical of the invention”, “fine chemical” or “the fine chemical” mean in context of any of the paragraphs [0014.1.15.15] to [0514.1.7.15] pyruvate, its salts, ester or ether in free form or bound form. In a preferred embodiment, the term “the fine chemical” means pyruvate or its salts, ester or ether, in free form or bound form.

In a preferred embodiment “pyruvate” means pyruvate in free form.

Accordingly, in one embodiment, the terms “fine chemical of the invention”, “fine chemical” or “the fine chemical” mean in context of any of the paragraphs [0014.1.15.15] to [0514.1.7.15] succinate, its salts, ester or ether in free form or bound form. In a preferred embodiment, the term “the fine chemical” means succinate or its salts, ester or ether, in free form or bound form.

In a preferred embodiment “succinate” means succinate in free form.

Accordingly, in one embodiment, the terms “fine chemical of the invention”, “fine chemical” or “the fine chemical” mean in context of any of the paragraphs [0014.1.15.15] to [0514.1.7.15] vitamin c and/or threonic acid, its salts, ester or ether in free form or bound form. In a preferred embodiment, the term “the fine chemical” means vitamin c and/or threonic acid or its salts, ester or ether, in free form or bound form.

In a preferred embodiment “vitamin c and/or threonic acid ” means vitamin c and/or threonic acid in free form.

In one embodiment, the term “the fine chemical” or “the respective fine chemical” mean in context of any of the paragraphs [0014.1.15.15] to [0514.1.7.15] at least one substrate or product of the citric acid cycle, a derivate of one of the substrates or products (for example pyruvate and malate as derivates of citramalate, acetyl-CoA as derivate of pyruvate or citrate, pyruvate as derivate of glycerate) or vitamin c, ascorbic acid or ascorbate and derivates like oxalate and/or threonic acid Further, the term “in context of any of the paragraphs [0014.1.15.15] to [0514.1.7.15]” as used herein means that for any of said paragraphs [0014.1.15.15] to [0514.1.7.15] the term “the fine chemical” is understood to follow the definition of section [0014.1.15.15] or section [0015.1.15.15], independently whether it refers to any other paragraph or not and whether the reference recites the term “fine chemical” in an other context.

Thus, in cases where one or more paragraphs or sections are incorporated by reference into any of the present paragraphs [0014.1.15.15] to [0514.1.7.15], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to this definition of this paragraph [0015.1.15.15].

Further, the term “fine chemicals” as used herein relates to compositions comprising said fine chemical(s), i.e. comprising citramalate, citrate, fumarate, glycerate, malate, oxalate, pyruvate, succinate, and/or vitamin c and/or threonic acid, respectively.

Accordingly, the present invention relates to a process for the production of succinate, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of malate, which comprises
(a) increasing or generating one or more activities selected from the group consisting of At1g13930-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of At1g13930-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of malate, which comprises
(a) increasing or generating one or more activities selected from the group consisting of eukaryotic translation initiation factor 5 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of vitamin c and/or threonic acid, which comprises
(a) increasing or generating one or more activities selected from the group consisting of eukaryotic translation initiation factor 5 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of fumarate, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of fumarate, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of vitamin c and/or threonic acid, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of fumarate, which comprises
(a) increasing or generating one or more activities selected from the group consisting of CBL-interacting protein kinase in a non-human organism or a part thereof, preferably a microorganism organism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of CBL-interacting protein kinase in a non-human organism or a part thereof, preferably a microorganism organism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of CBL-interacting protein kinase in a non-human organism or a part thereof, preferably a microorganism organism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating one or more activities selected from the group consisting of phenylalanine ammonia-lyase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating one or more activities selected from the group consisting of thioredoxin reductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of succinate, which comprises
(a) increasing or generating one or more activities selected from the group consisting of citrate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citramalate, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of succinate, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of malate, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating one or more activities selected from the group consisting of thioredoxin family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of thioredoxin family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of glycerate, which comprises
(a) increasing or generating one or more activities selected from the group consisting of thioredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

Accordingly, the present invention relates to a process for the production of fumarate, which comprises
(a) increasing or generating one or more activities selected from the group consisting of auxin response factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of auxin response factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of citramalate, which comprises
(a) increasing or generating one or more activities selected from the group consisting of monthiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating one or more activities selected from the group consisting of monthiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of glycerate, which comprises
(a) increasing or generating one or more activities selected from the group consisting of monthiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

Accordingly, the present invention relates to a process for the production of malate, which comprises
(a) increasing or generating one or more activities selected from the group consisting of monthiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of monthiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of vitamin c and/or threonic acid, which comprises
(a) increasing or generating one or more activities selected from the group consisting of monthiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of citramalate, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of glycerate, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

Accordingly, the present invention relates to a process for the production of malate, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of glycerate, which comprises
(a) increasing or generating one or more activities selected from the group consisting of thioredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

Accordingly, the present invention relates to a process for the production of citramalate, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of glycerate, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

Accordingly, the present invention relates to a process for the production of malate, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of vitamin c and/or threonic acid, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of fumarate, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of glycerate, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

Accordingly, the present invention relates to a process for the production of malate, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citramalate, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of vitamin c and/or threonic acid, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of glycerate, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

Accordingly, the present invention relates to a process for the production of citramalate, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of succinate, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citramalate, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of vitamin c and/or threonic acid, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of malate, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of fumarate, which comprises
(a) increasing or generating one or more activities selected from the group consisting of heat shock transcription factor in a non-human organism or a part thereof, preferably a microorganism organism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of heat shock transcription factor in a non-human organism or a part thereof, preferably a microorganism organism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of heat shock transcription factor in a non-human organism or a part thereof, preferably a microorganism organism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of glycerate, which comprises
(a) increasing or generating one or more activities selected from the group consisting of gibberellin 20-oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

Accordingly, the present invention relates to a process for the production of malate, which comprises
(a) increasing or generating one or more activities selected from the group consisting of gibberellin 20-oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of malate, which comprises
(a) increasing or generating one or more activities selected from the group consisting of translation initiation factor subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of fumarate, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of succinate, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating one or more activities selected from the group consisting of zinc finger protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of citramalate, which comprises
(a) increasing or generating one or more activities selected from the group consisting of CDP-diacylglycerol-serine O-phosphatidyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating one or more activities selected from the group consisting of CDP-diacylglycerol-serine O-phosphatidyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of CDP-diacylglycerol-serine O-phosphatidyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of malate, which comprises
(a) increasing or generating one or more activities selected from the group consisting of Glycine cleavage system T aminomethyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of malate, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of malate, which comprises
(a) increasing or generating one or more activities selected from the group consisting of adenylylsulfate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of adenylylsulfate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating one or more activities selected from the group consisting of sec-independent protein translocase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of sec-independent protein translocase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of fumarate, which comprises
(a) increasing or generating one or more activities selected from the group consisting of fumarate hydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of succinate, which comprises
(a) increasing or generating one or more activities selected from the group consisting of transcriptional regulator protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of glycerate, which comprises
(a) increasing or generating one or more activities selected from the group consisting of beta-hydroxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

Accordingly, the present invention relates to a process for the production of succinate, which comprises
(a) increasing or generating one or more activities selected from the group consisting of 30S ribosomal protein S3 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citramalate, which comprises
(a) increasing or generating one or more activities selected from the group consisting of 50S ribosomal protein L14 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of malate, which comprises
(a) increasing or generating one or more activities selected from the group consisting of acyl-CoA synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of citramalate, which comprises
(a) increasing or generating one or more activities selected from the group consisting of 2-oxoglutarate dehydrogenase E1 subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating one or more activities selected from the group consisting of 2-oxoglutarate dehydrogenase E1 subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of 2-oxoglutarate dehydrogenase E1 subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of 2-oxoglutarate dehydrogenase E1 subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of vitamin c and/or threonic acid, which comprises
(a) increasing or generating one or more activities selected from the group consisting of 2-oxoglutarate dehydrogenase E1 subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of citramalate, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating one or more activities selected from the group consisting of CTP synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of CTP synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of CTP synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of succinate, which comprises
(a) increasing or generating one or more activities selected from the group consisting of recombinase A in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of malate, which comprises
(a) increasing or generating one or more activities selected from the group consisting of glucose-1-phosphate cytidylyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of fumarate, which comprises
(a) increasing or generating one or more activities selected from the group consisting of CDP-glucose-4,6-dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of CDP-glucose-4,6-dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of malate, which comprises
(a) increasing or generating one or more activities selected from the group consisting of aminotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of isocitrate lyase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating one or more activities selected from the group consisting of NADH-quinone oxidoreductase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of NADH-quinone oxidoreductase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of malate, which comprises
(a) increasing or generating one or more activities selected from the group consisting of RNA polymerase sigma factor in a non-human organism or a part thereof, preferably a microorganism organism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of malate, which comprises
(a) increasing or generating one or more activities selected from the group consisting of hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of malate, which comprises
(a) increasing or generating one or more activities selected from the group consisting of glutamate-ammonia-ligase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of electron transfer flavoprotein subunit beta in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating one or more activities selected from the group consisting of AX653549-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of AX653549-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of fumarate, which comprises
(a) increasing or generating one or more activities selected from the group consisting of AY087308-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of AY087308-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of AY087308-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating one or more activities selected from the group consisting of fumarate hydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of threonine synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of fumarate, which comprises
(a) increasing or generating one or more activities selected from the group consisting of ribulokinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating one or more activities selected from the group consisting of ribulokinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citrate, which comprises
(a) increasing or generating one or more activities selected from the group consisting of glucose dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrate or a composition comprising citrate in said non-human organism or in the culture medium surrounding said non-human organism.

Accordingly, the present invention relates to a process for the production of fumarate, which comprises
(a) increasing or generating one or more activities selected from the group consisting of acyl-CoA dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of citrate, which comprises
(a) increasing or generating one or more activities selected from the group consisting of betaine aldehyde dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrate or a composition comprising citrate in said non-human organism or in the culture medium surrounding said non-human organism.

Accordingly, the present invention relates to a process for the production of malate, which comprises
(a) increasing or generating one or more activities selected from the group consisting of ATP-binding component of a transport system in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of malate, which comprises
(a) increasing or generating one or more activities selected from the group consisting of membrane transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of membrane transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citrate, which comprises
(a) increasing or generating one or more activities selected from the group consisting of glycerate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrate or a composition comprising citrate in said non-human organism or in the culture medium surrounding said non-human organism.

Accordingly, the present invention relates to a process for the production of fumarate, which comprises
(a) increasing or generating one or more activities selected from the group consisting of isochorismate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of isochorismate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of fumarate, which comprises
(a) increasing or generating one or more activities selected from the group consisting of zinc transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating one or more activities selected from the group consisting of b0801-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of malate, which comprises
(a) increasing or generating one or more activities selected from the group consisting of leucyl/phenylalanyl-tRNA-protein transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of vitamin c and/or threonic acid, which comprises
(a) increasing or generating one or more activities selected from the group consisting of methylglyoxal synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of malate, which comprises
(a) increasing or generating one or more activities selected from the group consisting of b1003-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of malate, which comprises
(a) increasing or generating one or more activities selected from the group consisting of membrane protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of b1285-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of malate, which comprises
(a) increasing or generating one or more activities selected from the group consisting of phenylacetic acid degradation protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of malate, which comprises
(a) increasing or generating one or more activities selected from the group consisting of b1425-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of lipoprotein precursor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citramalate, which comprises
(a) increasing or generating one or more activities selected from the group consisting of b1445-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of citramalate, which comprises
(a) increasing or generating one or more activities selected from the group consisting of b1522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of malate, which comprises
(a) increasing or generating one or more activities selected from the group consisting of b1522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of fumarate, which comprises
(a) increasing or generating one or more activities selected from the group consisting of Fe—S subunit of oxidoreductase in a non-human organism or a part thereof, preferably a microorganism organism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of fumarate, which comprises
(a) increasing or generating one or more activities selected from the group consisting of fumarate hydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating one or more activities selected from the group consisting of electron transport complex protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of succinate, which comprises
(a) increasing or generating one or more activities selected from the group consisting of methylglyoxalase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citramalate, which comprises
(a) increasing or generating one or more activities selected from the group consisting of b1670-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of malate, which comprises
(a) increasing or generating one or more activities selected from the group consisting of b1670-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of citramalate, which comprises
(a) increasing or generating one or more activities selected from the group consisting of b1672-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of malate, which comprises
(a) increasing or generating one or more activities selected from the group consisting of b1672-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of vitamin c and/or threonic acid, which comprises
(a) increasing or generating one or more activities selected from the group consisting of b1672-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of malate, which comprises
(a) increasing or generating one or more activities selected from the group consisting of b1742-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of b1742-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of succinate, which comprises
(a) increasing or generating one or more activities selected from the group consisting of arginine N-succinyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of vitamin c and/or threonic acid, which comprises
(a) increasing or generating one or more activities selected from the group consisting of ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of malate, which comprises
(a) increasing or generating one or more activities selected from the group consisting of protease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of vitamin c and/or threonic acid, which comprises
(a) increasing or generating one or more activities selected from the group consisting of dATP pyrophosphohydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of succinate, which comprises
(a) increasing or generating one or more activities selected from the group consisting of b1898-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating one or more activities selected from the group consisting of flagellar M-ring protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of flagellar M-ring protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of flagellar M-ring protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of malate, which comprises
(a) increasing or generating one or more activities selected from the group consisting of O-antigen chain length determinant in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of fumarate, which comprises
(a) increasing or generating one or more activities selected from the group consisting of b2399-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating one or more activities selected from the group consisting of b2399-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of vitamin c and/or threonic acid, which comprises
(a) increasing or generating one or more activities selected from the group consisting of b2399-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of vitamin c and/or threonic acid, which comprises
(a) increasing or generating one or more activities selected from the group consisting of cysteine synthase A in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of vitamin c and/or threonic acid, which comprises
(a) increasing or generating one or more activities selected from the group consisting of cysteine synthase B in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of fumarate, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of murein transglycosylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of succinate, which comprises
(a) increasing or generating one or more activities selected from the group consisting of 2-C-methyl-D-erythritol 4-phosphate cytidylyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of succinate, which comprises
(a) increasing or generating one or more activities selected from the group consisting of amino-acid acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of succinate, which comprises
(a) increasing or generating one or more activities selected from the group consisting of xanthine dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of succinate, which comprises
(a) increasing or generating one or more activities selected from the group consisting of cystathionine beta-lyase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating one or more activities selected from the group consisting of methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of fumarate, which comprises
(a) increasing or generating one or more activities selected from the group consisting of lipopolysaccharide core biosynthesis protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating one or more activities selected from the group consisting of b3777-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of malate, which comprises
(a) increasing or generating one or more activities selected from the group consisting of b3791-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of b3791-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citramalate, which comprises
(a) increasing or generating one or more activities selected from the group consisting of b3814-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating one or more activities selected from the group consisting of b3817-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of citramalate, which comprises
(a) increasing or generating one or more activities selected from the group consisting of threonine efflux protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of malate, which comprises
(a) increasing or generating one or more activities selected from the group consisting of threonine efflux protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of b3989-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of fumarate, which comprises
(a) increasing or generating one or more activities selected from the group consisting of b4029-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of b4029-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of b4029-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating one or more activities selected from the group consisting of b4050-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of b4050-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of citramalate, which comprises
(a) increasing or generating one or more activities selected from the group consisting of b4056-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of citrate, which comprises
(a) increasing or generating one or more activities selected from the group consisting of aspartase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrate or a composition comprising citrate in said non-human organism or in the culture medium surrounding said non-human organism.

Accordingly, the present invention relates to a process for the production of fumarate, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of vitamin c and/or threonic acid, which comprises
(a) increasing or generating one or more activities selected from the group consisting of purine-nucleoside phosphorylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of succinate, which comprises
(a) increasing or generating one or more activities selected from the group consisting of lysine decarboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of succinate, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating one or more activities selected from the group consisting of GM02LC17485-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of citramalate, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of citramalate, which comprises
(a) increasing or generating one or more activities selected from the group consisting of GM02LC5744-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating one or more activities selected from the group consisting of GM02LC5744-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of citramalate, which comprises
(a) increasing or generating one or more activities selected from the group consisting of heat shock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating one or more activities selected from the group consisting of agmatinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of ABC transporter ATP binding component in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of ABC transporter ATP binding component in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating one or more activities selected from the group consisting of oxireductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating one or more activities selected from the group consisting of phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citramalate, which comprises
(a) increasing or generating one or more activities selected from the group consisting of CDP-diacylglycerol-glycerol-3-phosphate 3- phosphatidyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of malate, which comprises
(a) increasing or generating one or more activities selected from the group consisting of CDP-diacylglycerol-glycerol-3-phosphate 3- phosphatidyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of adenylate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citramalate, which comprises
(a) increasing or generating one or more activities selected from the group consisting of cation-transporting ATPase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of malate, which comprises
(a) increasing or generating one or more activities selected from the group consisting of cation-transporting ATPase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of fumarate, which comprises
(a) increasing or generating one or more activities selected from the group consisting of fumarate hydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of citramalate, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating one or more activities selected from the group consisting of bifunctional purine biosynthesis protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of citramalate, which comprises
(a) increasing or generating one or more activities selected from the group consisting of malate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of succinate, which comprises
(a) increasing or generating one or more activities selected from the group consisting of malate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of glycerate, which comprises
(a) increasing or generating one or more activities selected from the group consisting of dihydrolipoamide dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

Accordingly, the present invention relates to a process for the production of succinate, which comprises
(a) increasing or generating one or more activities selected from the group consisting of dihydrolipoamide dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of succinate, which comprises
(a) increasing or generating one or more activities selected from the group consisting of slr1170-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating one or more activities selected from the group consisting of phosphate import ATP-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating one or more activities selected from the group consisting of gamma-glutamyltranspeptidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of malate, which comprises
(a) increasing or generating one or more activities selected from the group consisting of photosystem II protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of fumarate, which comprises
(a) increasing or generating one or more activities selected from the group consisting of malonyl CoA-acyl carrier protein transacylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating one or more activities selected from the group consisting of malonyl CoA-acyl carrier protein transacylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of glycerate, which comprises
(a) increasing or generating one or more activities selected from the group consisting of photosystem I reaction centre subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

Accordingly, the present invention relates to a process for the production of fumarate, which comprises
(a) increasing or generating one or more activities selected from the group consisting of Sec-independent protein translocase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating one or more activities selected from the group consisting of Sec-independent protein translocase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citramalate, which comprises
(a) increasing or generating one or more activities selected from the group consisting of 2-isopropylmalate synthase in a non-human organism or a part thereof, preferably a microorganism organism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating one or more activities selected from the group consisting of multiple antibiotic resistance protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating one or more activities selected from the group consisting of multiple antibiotic resistance protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating one or more activities selected from the group consisting of TTC1386-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of TTC1386-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of TTC1386-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citramalate, which comprises
(a) increasing or generating one or more activities selected from the group consisting of oxidoreductase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of glycerate, which comprises
(a) increasing or generating one or more activities selected from the group consisting of oxidoreductase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

Accordingly, the present invention relates to a process for the production of malate, which comprises
(a) increasing or generating one or more activities selected from the group consisting of oxidoreductase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of fumarate, which comprises
(a) increasing or generating one or more activities selected from the group consisting of XM_{—}473199-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of XM_{—}473199-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of XM_{—}473199-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of succinate, which comprises
(a) increasing or generating one or more activities selected from the group consisting of glutamate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citramalate, which comprises
(a) increasing or generating one or more activities selected from the group consisting of acetyl-CoA hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of pyruvate, which comprises
(a) increasing or generating one or more activities selected from the group consisting of acetyl-CoA hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of pyruvate or a composition comprising pyruvate in said non-human organism or in the culture medium surrounding said non-human organism.

Accordingly, the present invention relates to a process for the production of malate, which comprises
(a) increasing or generating one or more activities selected from the group consisting of pyruvate carboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of fumarate, which comprises
(a) increasing or generating one or more activities selected from the group consisting of ycr102c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of fumarate, which comprises
(a) increasing or generating one or more activities selected from the group consisting of YDL159W-A-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of fumarate, which comprises
(a) increasing or generating one or more activities selected from the group consisting of coproporphyrinogen III oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating one or more activities selected from the group consisting of coproporphyrinogen III oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of malate, which comprises
(a) increasing or generating one or more activities selected from the group consisting of ygl237c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of ygl237c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating one or more activities selected from the group consisting of YGR193C-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of YGR193C-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of YGR193C-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of malate, which comprises
(a) increasing or generating one or more activities selected from the group consisting of ygr221 c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of citrate, which comprises
(a) increasing or generating one or more activities selected from the group consisting of glycerol kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrate or a composition comprising citrate in said non-human organism or in the culture medium surrounding said non-human organism.

Accordingly, the present invention relates to a process for the production of malate, which comprises
(a) increasing or generating one or more activities selected from the group consisting of yhr131c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of yhr207c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of glycerate, which comprises
(a) increasing or generating one or more activities selected from the group consisting of DnaJ-like chaperone in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

Accordingly, the present invention relates to a process for the production of oxalate, which comprises
(a) increasing or generating one or more activities selected from the group consisting of hexose transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oxalate or a composition comprising oxalate in said non-human organism or in the culture medium surrounding said non-human organism.

Accordingly, the present invention relates to a process for the production of citramalate, which comprises
(a) increasing or generating one or more activities selected from the group consisting of branched chain aminotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating one or more activities selected from the group consisting of polygalacturonase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of polygalacturonase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of malate, which comprises
(a) increasing or generating one or more activities selected from the group consisting of YKL038W-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of vitamin c and/or threonic acid, which comprises
(a) increasing or generating one or more activities selected from the group consisting of YKL140W-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of fumarate, which comprises
(a) increasing or generating one or more activities selected from the group consisting of ylr065c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating one or more activities selected from the group consisting of isocitrate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating one or more activities selected from the group consisting of ylr326w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of glycerate, which comprises
(a) increasing or generating one or more activities selected from the group consisting of ylr326w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

Accordingly, the present invention relates to a process for the production of vitamin c and/or threonic acid, which comprises
(a) increasing or generating one or more activities selected from the group consisting of ylr326w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of fumarate, which comprises
(a) increasing or generating one or more activities selected from the group consisting of yml084w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating one or more activities selected from the group consisting of yml084w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citramalate, which comprises
(a) increasing or generating one or more activities selected from the group consisting of yol073c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of malate, which comprises
(a) increasing or generating one or more activities selected from the group consisting of yol073c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of vitamin c and/or threonic acid, which comprises
(a) increasing or generating one or more activities selected from the group consisting of yol160w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of malate, which comprises
(a) increasing or generating one or more activities selected from the group consisting of acetyl-CoA acetyltransferase in a non-human organism or a part thereof, preferably a microorganism organism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of vitamin c and/or threonic acid, which comprises
(a) increasing or generating one or more activities selected from the group consisting of ammonium transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of fumarate, which comprises
(a) increasing or generating one or more activities selected from the group consisting of 47266012-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of 47266012-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of 47266012-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating one or more activities selected from the group consisting of 49747384_SOYBEAN-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating one or more activities selected from the group consisting of 49747384_SOYBEAN-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating one or more activities selected from the group consisting of 49747384_SOYBEAN-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of vitamin c and/or threonic acid, which comprises
(a) increasing or generating one or more activities selected from the group consisting of 49747384_SOYBEAN-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of citramalate, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of succinate, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citramalate, which comprises
(a) increasing or generating one or more activities selected from the group consisting of glucose-6-phosphate 1-dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

An embodiment of the present invention relates to a process for the production of citramalate, which comprises
(a1) increasing or generating the activity of a polypeptide comprising a polypeptide as depicted in a respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical citramalate; or
(a2) increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical citramalate; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of citramalate or a composition comprising citramalate in said non-human organism or in the culture medium surrounding said non-human organism.

An embodiment of the present invention relates to a process for the production of citrate, which comprises
(a1) increasing or generating the activity of a polypeptide comprising a polypeptide as depicted in a respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical citrate; or
(a2) increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical citrate; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of citrate or a composition comprising citrate in said non-human organism or in the culture medium surrounding said non-human organism.

An embodiment of the present invention relates to a process for the production of fumarate, which comprises
(a1) increasing or generating the activity of a polypeptide comprising a polypeptide as depicted in a respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical fumarate; or
(a2) increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical fumarate; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of fumarate or a composition comprising fumarate in said non-human organism or in the culture medium surrounding said non-human organism.

An embodiment of the present invention relates to a process for the production of glycerate, which comprises
(a1) increasing or generating the activity of a polypeptide comprising a polypeptide as depicted in a respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical glycerate; or
(a2) increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical glycerate; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of glycerate or a composition comprising glycerate in said non-human organism or in the culture medium surrounding said non-human organism.

An embodiment of the present invention relates to a process for the production of malate, which comprises
(a1) increasing or generating the activity of a polypeptide comprising a polypeptide as depicted in a respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical malate; or
(a2) increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical malate; or
(a3) increasing or generating the activity of a functional equivalent of (a1) or (a2);

in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of malate or a composition comprising malate in said non-human organism or in the culture medium surrounding said non-human organism.

An embodiment of the present invention relates to a process for the production of oxalate, which comprises
(a1) increasing or generating the activity of a polypeptide comprising a polypeptide as depicted in a respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical oxalate; or
(a2) increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical oxalate; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of oxalate or a composition comprising oxalate in said non-human organism or in the culture medium surrounding said non-human organism.

An embodiment of the present invention relates to a process for the production of pyruvate, which comprises
(a1) increasing or generating the activity of a polypeptide comprising a polypeptide as depicted in a respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical pyruvate; or
(a2) increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical pyruvate; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of pyruvate or a composition comprising pyruvate in said non-human organism or in the culture medium surrounding said non-human organism.

An embodiment of the present invention relates to a process for the production of succinate, which comprises
(a1) increasing or generating the activity of a polypeptide comprising a polypeptide as depicted in a respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical succinate; or
(a2) increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical succinate; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of succinate or a composition comprising succinate in said non-human organism or in the culture medium surrounding said non-human organism.

An embodiment of the present invention relates to a process for the production of vitamin c and/or threonic acid, which comprises
(a1) increasing or generating the activity of a polypeptide comprising a polypeptide as depicted in a respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical vitamin c and/or threonic acid; or
(a2) increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical vitamin c and/or threonic acid; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of vitamin c and/or threonic acid or a composition comprising vitamin c and/or threonic acid in said non-human organism or in the culture medium surrounding said non-human organism.

Accordingly, the increase or generation of one or more said activities is for example conferred by one or more expression products of said nucleic acid molecule, e.g. proteins. Accordingly, in the present invention described above, the increase or generation of one or more said activities is for example conferred by one or more protein(s) each comprising a polypeptide selected from the group as depicted in Table II, application no. 15, column 5 and 8, or a homolog or a fragment thereof.

The process of the invention comprises in one embodiment the following steps:
(i) increasing or generating of the expression of; and/or
(ii) increasing or generating the expression of an expression product of; and/or
(iii) increasing or generating one or more activities of an expression product encoded by;
   at least one nucleic acid molecule (in the following “Fine Chemical Related Protein (FCRP)”-encoding gene or “FCRP”-gene) comprising a nucleic acid molecule selected from the group consisting of:
(a) a nucleic acid molecule encoding the polypeptide shown in column 5 or 8 of Table II, preferably Table II B, application no. 15, or a homolog or a fragment thereof;
(b) a nucleic acid molecule shown in column 5 or 8 of Table I, preferably Table I B, application no. 15, or a homolog or a fragment thereof (preferably the coding region thereof);
(c) a nucleic acid molecule, which, as a result of the degeneracy of the genetic code, can be derived from a polypeptide sequence depicted in column 5 or 8 of Table II, preferably Table II B, application no. 15;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 15, or the coding region thereof;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d),
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 15;
(i) a nucleic acid molecule encoding a polypeptide having the activity represented by a respective protein as depicted in column 5 of Table II, application no. 15;
(j) a nucleic acid molecule which comprises a polynucleotide, which is obtained by amplifying a cDNA library or a genomic library using the primers as depicted in column 8 of Table III, application no. 15; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment thereof said nucleic acid molecule encodes a polypeptide which has the activity of the polypeptide represented by a protein comprising a polypeptide as depicted in the corresponding hit in column 5 of Table II, application no. 15.

In another preferred embodiment thereof said nucleic acid molecule confers a generation or an increase of the respective fine chemical in a non-human organism or a part thereof as compared to a corresponding wild-type non-human organism or part thereof.

In another preferred embodiment thereof said nucleic acid molecule encodes a polypeptide which has the activity of the polypeptide represented by a protein as depicted in the corresponding hit in column 5 of Table II, application no. 15, and confers a generation or an increase of the respective fine chemical in a non-human organism or a part thereof as compared to a corresponding wild-type non-human organism or part thereof.

Accordingly, the genes of the present invention or used in accordance with the present invention, which respectively encode a protein having an activity of 2-C-methyl-D-erythritol 4-phosphate cytidylyltransferase, 2-isopropylmalate synthase, 2-oxoglutarate dehydrogenase E1 subunit, 30S ribosomal protein S3, 47266012-protein, 49747384_SOYBEAN-protein, 50S ribosomal protein L14, ABC transporter ATP binding component, ABC transporter permease protein, acetyl-CoA acetyltransferase, acetyl-CoA hydrolase, acetyltransferase, acyl-CoA dehydrogenase, acyl-CoA synthase, adenylate kinase, adenylylsulfate kinase, agmatinase, amino-acid acetyltransferase, aminotransferase, ammonium transporter, arginine N-succinyltransferase, aspartase, At1g13930-protein, ATP-binding component of a transport system, auxin response factor, AX653549-protein, AY087308-protein, b0801-protein, b1003-protein, b1285-protein, b1425-protein, b1445-protein, b1522-protein, b1670-protein, b1672-protein, b1742-protein, b1898-protein, b2399-protein, b3777-protein, b3791-protein, b3814-protein, b3817-protein, b3989-protein, b4029-protein, b4050-protein, b4056-protein, beta-hydroxylase, betaine aldehyde dehydrogenase, bifunctional purine biosynthesis protein, branched chain aminotransferase, calcium-dependent protein kinase, cation-transporting ATPase, CBL-interacting protein kinase, CDP-diacylglycerol-glycerol-3-phosphate 3-phosphatidyltransferase, CDP-diacylglycerol-serine O-phosphatidyltransferase, CDP-glucose-4,6-dehydratase, citrate synthase, coproporphyrinogen III oxidase, CTP synthase, cystathionine beta-lyase, cysteine synthase A, cysteine synthase B, dATP pyrophosphohydrolase, dihydrolipoamide dehydrogenase, DnaJ-like chaperone, electron transfer flavoprotein subunit beta, electron transport complex protein, eukaryotic translation initiation factor 5, Fe—S subunit of oxidoreductase, flagellar M-ring protein, fumarate hydratase, gamma-glutamyltranspeptidase, gibberellin 20-oxidase, glucose dehydrogenase, glucose-1-phosphate cytidylyltransferase, glucose-6-phosphate 1-dehydrogenase, glutamate dehydrogenase, glutamate-ammonia-ligase, glutaredoxin, glycerate kinase, glycerol kinase, Glycine cleavage system T aminomethyltransferase, GM02LC17485-protein, GM02LC5744-protein, heat shock protein, heat shock transcription factor, hexose transporter, hydrolase, isochorismate synthase, isocitrate dehydrogenase, isocitrate lyase, leucyl/phenylalanyl-tRNA-protein transferase, lipopolysaccharide core biosynthesis protein, lipoprotein precursor, lysine decarboxylase, malate dehydrogenase, malonyl CoA-acyl carrier protein transacylase, membrane protein, membrane transport protein, methylglyoxal synthase, methylglyoxalase, methyltransferase, monothiol glutaredoxin, monthiol glutaredoxin, multiple antibiotic resistance protein, murein transglycosylase, NADH-quinone oxidoreductase subunit, O-antigen chain length determinant, oxidoreductase, oxidoreductase subunit, oxireductase, phenylacetic acid degradation protein, phenylalanine ammonia-lyase, phosphatase, phosphate import ATP-binding protein, photosystem I reaction centre subunit, photosystem II protein, polygalacturonase, protease, protein phosphatase, purine-nucleoside phosphorylase, pyruvate carboxylase, pyruvate kinase, recombinase A, ribulokinase, RNA polymerase sigma factor, sec-independent protein translocase, Sec-independent protein translocase subunit, slr1170-protein, thioredoxin, thioredoxin family protein, thioredoxin reductase, threonine efflux protein, threonine synthase, transcription factor, transcriptional regulator, transcriptional regulator protein, translation initiation factor subunit, TTC1386-protein, xanthine dehydrogenase, XM_{—}473199-protein, ycr102c-protein, YDL159W-A-protein, ygl237c-protein, YGR193C-protein, ygr221 c-protein, yhr131c-protein, yhr207c-protein, YKL038W-protein, YKL140W-protein, ylr065c-protein, ylr326w-protein, yml084w-protein, yol073c-protein, yol160w-protein, zinc finger protein, or zinc transporter, which respectively encode a protein comprising a polypeptide encoded by a nucleic acid sequence as shown in Table I, application no. 15, column 5 or 8, (preferably the coding region thereof), or a homolog or a fragment thereof, which respectively encode a protein comprising a polypeptide as depicted in Table II, application no. 15, column 5 or 8, or a homolg or a fragment thereof, and/or which respectively can be amplified with the primer set shown in Table III, application no. 15, column 8, are also referred to as “FCRP genes”.

Proteins or polypeptides encoded by “FCRP-genes” are referred to as “Fine Chemical Related Proteins” or “FCRP”. For the purposes of the description of the present invention, the respective protein having an activity of 2-C-methyl-D-erythritol 4-phosphate cytidylyltransferase, 2-isopropylmalate synthase, 2-oxoglutarate dehydrogenase E1 subunit, 30S ribosomal protein S3, 47266012-protein, 49747384_SOYBEAN-protein, 50S ribosomal protein L14, ABC transporter ATP binding component, ABC transporter permease protein, acetyl-CoA acetyltransferase, acetyl-CoA hydrolase, acetyltransferase, acyl-CoA dehydrogenase, acyl-CoA synthase, adenylate kinase, adenylylsulfate kinase, agmatinase, amino-acid acetyltransferase, aminotransferase, ammonium transporter, arginine N-succinyltransferase, aspartase, Atl g13930-protein, ATP-binding component of a transport system, auxin response factor, AX653549-protein, AY087308-protein, b0801-protein, b1003-protein, b1285-protein, b1425-protein, b1445-protein, b1522-protein, b1670-protein, b1672-protein, b1742-protein, b1898-protein, b2399-protein, b3777-protein, b3791-protein, b3814-protein, b3817-protein, b3989-protein, b4029-protein, b4050-protein, b4056-protein, beta-hydroxylase, betaine aldehyde dehydrogenase, bifunctional purine biosynthesis protein, branched chain aminotransferase, calcium-dependent protein kinase, cation-transporting ATPase, CBL-interacting protein kinase, CDP-diacylglycerol-glycerol-3-phosphate 3- phosphatidyltransferase, CDP-diacylglycerolserine O-phosphatidyltransferase, CDP-glucose-4,6-dehydratase, citrate synthase, coproporganism phyrinogen III oxidase, CTP synthase, cystathionine beta-lyase, cysteine synthase A, cysteine synthase B, dATP pyrophosphohydrolase, dihydrolipoamide dehydrogenase, DnaJ-like chaperone, electron transfer flavoprotein subunit beta, electron transport complex protein, eukaryotic translation initiation factor 5, Fe—S subunit of oxidoreductase, flagellar M-ring protein, fumarate hydratase, gamma-glutamyltranspeptidase, gibberellin 20-oxidase, glucose dehydrogenase, glucose-1-phosphate cytidylyltransferase, glucose-6-phosphate 1-dehydrogenase, glutamate dehydrogenase, glutamate-ammonia-ligase, glutaredoxin, glycerate kinase, glycerol kinase, Glycine cleavage system T aminomethyltransferase, GM02LC17485-protein, GM02LC5744-protein, heat shock protein, heat shock transcription factor, hexose transporter, hydrolase, isochorismate synthase, isocitrate dehydrogenase, isocitrate lyase, leucyl/phenylalanyl-tRNA-protein transferase, lipopolysaccharide core biosynthesis protein, lipoprotein precursor, lysine decarboxylase, malate dehydrogenase, malonyl CoA-acyl carrier protein transacylase, membrane protein, membrane transport protein, methylglyoxal synthase, methylglyoxalase, methyl-transferase, monothiol glutaredoxin, monthiol glutaredoxin, multiple antibiotic resistance protein, murein transglycosylase, NADH-quinone oxidoreductase subunit, O-antigen chain length determinant, oxidoreductase, oxidoreductase subunit, oxireductase, phenylacetic acid degradation protein, phenylalanine ammonia-lyase, phosphatase, phosphate import ATP-binding protein, photosystem I reaction centre subunit, photosystem II protein, polygalacturonase, protease, protein phosphatase, purine-nucleoside phosphorylase, pyruvate carboxylase, pyruvate kinase, recombinase A, ribulokinase, RNA polymerase sigma factor, sec-independent protein translocase, Sec-independent protein translocase subunit, slr1170-protein, thioredoxin, thioredoxin family protein, thioredoxin reductase, threonine efflux protein, threonine synthase, transcription factor, transcriptional regulator, transcriptional regulator protein, translation initiation factor subunit, TTC1386-protein, xanthine dehydrogenase, XM_{—}473199-protein, ycr102c-protein, YDL159W-A-protein, ygl237c-protein, YGR193C-protein, ygr221 c-protein, yhrl 31 c-protein, yhr207c-protein, YKL038W-protein, YKL140W-protein, ylr065c-protein, ylr326w-protein, yml084w-protein, yol073c-protein, yol160w-protein, zinc finger protein, or zinc transporter, the respective protein comprising a polypeptide encoded by one or more respective nucleic acid sequences as shown in Table I, application no. 15, column 5 or 8, (preferably the coding region thereof), or a homolog or fragment thereof, the respective protein comprising a respective polypeptide as depicted in Table II, application no. 15, column 5 or 8, or a homolog or fragment thereof, the respective protein comprising a sequence corresponding to the consensus sequence as shown in Table IV, application no. 15, column 8, and/or the respective protein comprising at least one polypeptide motif as shown in Table IV, application no. 15, column 8 is also referred to as Fine Chemical Related Protein” or “FCRP”.

Thus, in one embodiment, the present invention provides a process of the production of citramalate, citrate, fumarate, glycerate, malate, oxalate, pyruvate, succinate, and/or vitamin c and/or threonic acid, by increasing or generating one or more activities, especially selected from the group consisting of 2-C-methyl-D-erythritol 4-phosphate cytidylyltransferase, 2-isopropylmalate synthase, 2-oxoglutarate dehydrogenase E1 subunit, 30S ribosomal protein S3, 47266012-protein, 49747384_SOYBEAN-protein, 50S ribosomal protein L14, ABC transporter ATP binding component, ABC transporter permease protein, acetyl-CoA acetyltransferase, acetyl-CoA hydrolase, acetyltransferase, acyl-CoA dehydrogenase, acyl-CoA synthase, adenylate kinase, adenylylsulfate kinase, agmatinase, amino-acid acetyltransferase, aminotransferase, ammonium transporter, arginine N-succinyltransferase, aspartase, At1g13930-protein, ATP-binding component of a transport system, auxin response factor, AX653549-protein, AY087308-protein, b0801-protein, b1003-protein, b1285-protein, b1425-protein, b1445-protein, b1522-protein, b1670-protein, b1672-protein, b1742-protein, b1898-protein, b2399-protein, b3777-protein, b3791-protein, b3814-protein, b3817-protein, b3989-protein, b4029-protein, b4050-protein, b4056-protein, beta-hydroxylase, betaine aldehyde dehydrogenase, bifunctional purine biosynthesis protein, branched chain aminotransferase, calcium-dependent protein kinase, cation-transporting ATPase, CBL-interacting protein kinase, CDP-diacylglycerol-glycerol-3-phosphate 3-phosphatidyltransferase, CDP-diacylglycerol-serine O-phosphatidyltransferase, CDP-glucose-4,6-dehydratase, citrate synthase, coproporphyrinogen III oxidase, CTP synthase, cystathionine beta-lyase, cysteine synthase A, cysteine synthase B, dATP pyrophosphohydrolase, dihydrolipoamide dehydrogenase, DnaJ-like chaperone, electron transfer flavoprotein subunit beta, electron transport complex protein, eukaryotic translation initiation factor 5, Fe—S subunit of oxidoreductase, flagellar M-ring protein, fumarate hydratase, gamma-glutamyltranspeptidase, gibberellin 20-oxidase, glucose dehydrogenase, glucose-1-phosphate cytidylyltransferase, glucose-6-phosphate 1-dehydrogenase, glutamate dehydrogenase, glutamate-ammonia-ligase, glutaredoxin, glycerate kinase, glycerol kinase, Glycine cleavage system T aminomethyltransferase, GM02LC17485-protein, GM02LC5744-protein, heat shock protein, heat shock transcription factor, hexose transporter, hydrolase, isochorismate synthase, isocitrate dehydrogenase, isocitrate lyase, leucyl/phenylalanyl-tRNA-protein transferase, lipopolysaccharide core biosynthesis protein, lipoprotein precursor, lysine decarboxylase, malate dehydrogenase, malonyl CoA-acyl carrier protein transacylase, membrane protein, membrane transport protein, methylglyoxal synthase, methylglyoxalase, methyltransferase, monothiol glutaredoxin, monthiol glutaredoxin, multiple antibiotic resistance protein, murein transglycosylase, NADH-quinone oxidoreductase subunit, O-antigen chain length determinant, oxidoreductase, oxidoreductase subunit, oxireductase, phenylacetic acid degradation protein, phenylalanine ammonialyase, phosphatase, phosphate import ATP-binding protein, photosystem I reaction centre subunit, photosystem 11-protein, polygalacturonase, protease, protein phosphatase, purinenucleoside phosphorylase, pyruvate carboxylase, pyruvate kinase, recombinase A, ribulokinase, RNA polymerase sigma factor, sec-independent protein translocase, Sec-independent protein translocase subunit, slr1170-protein, thioredoxin, thioredoxin family protein, thioredoxin reductase, threonine efflux protein, threonine synthase, transcription factor, transcriptional regulator, transcriptional regulator protein, translation initiation factor subunit, TTC1386-protein, xanthine dehydrogenase, XM_{—}473199-protein, ycr102c-protein, YDL159W-A-protein, ygl237c-protein, YGR193C-protein, ygr221c-protein, yhr131c-protein, yhr207c-protein, YKL038W-protein, YKL140W-protein, ylr065c-protein, ylr326w-protein, yml084w-protein, yol073c-protein, yol160w-protein, zinc finger protein, and zinc transporter, which is conferred by one or more FCRPs or the gene product of one or more FCRP-genes, for example by the gene product of a nucleic acid sequences comprising a polynucleotide selected from the group as shown in Table I, application no. 15, column 5 or 8, (preferably by the coding region thereof), or a homolog or a fragment thereof, e.g. or by one or more proteins each comprising a polypeptide encoded by one or more nucleic acid sequences selected from the group as shown in Table I, application no. 15, column 5 or 8, (preferably by the coding region thereof), or a homolog or a fragment thereof, or by one or more protein(s) each comprising a polypeptide selected from the group as depicted in Table II, application no. 15, column 5 and 8, or a homolog thereof, or a protein comprising a sequence corresponding to the consensus sequence or comprising at least one polypeptide motif as shown in Table IV, application no. 15, column 8.

for the disclosure of this paragraph see [0025.1.7.7] above

In an embodiment, the process comprises increasing or generating the activity of one or more polypeptides having said activity, e.g. by generating or increasing the amount and/or specific activity in the cell or a compartment of a cell of one of more FCRP, especially selected from the group consisting of 2-C-methyl-D-erythritol 4-phosphate cytidylyltransferase, 2-isopropylmalate synthase, 2-oxoglutarate dehydrogenase E1 subunit, 30S ribosomal protein S3, 47266012-protein, 49747384_SOYBEAN-protein, 50S ribosomal protein L14, ABC transporter ATP binding component, ABC transporter permease protein, acetyl-CoA acetyltransferase, acetyl-CoA hydrolase, acetyltransferase, acyl-CoA dehydrogenase, acyl-CoA synthase, adenylate kinase, adenylylsulfate kinase, agmatinase, amino-acid acetyltransferase, aminotransferase, ammonium transporter, arginine N-succinyltransferase, aspartase, At1g13930-protein, ATP-binding component of a transport system, auxin response factor, AX653549-protein, AY087308-protein, b0801-protein, b1003-protein, b1285-protein, b1425-protein, b1445-protein, b1522-protein, b1670-protein, b1672-protein, b1742-protein, b1898-protein, b2399-protein, b3777-protein, b3791-protein, b3814-protein, b3817-protein, b3989-protein, b4029-protein, b4050-protein, b4056-protein, beta-hydroxylase, betaine aldehyde dehydrogenase, bifunctional purine biosynthesis protein, branched chain aminotransferase, calcium-dependent protein kinase, cation-transporting ATPase, CBL-interacting protein kinase, CDP-diacylglycerol-glycerol-3-phosphate 3- phosphatidyltransferase, CDP-diacylglycerol-serine O-phosphatidyltransferase, CDP-glucose-4,6-dehydratase, citrate synthase, coproporphyrinogen III oxidase, CTP synthase, cystathionine beta-lyase, cysteine synthase A, cysteine synthase B, dATP pyrophosphohydrolase, dihydrolipoamide dehydrogenase, DnaJ-like chaperone, electron transfer flavoprotein subunit beta, electron transport complex protein, eukaryotic translation initiation factor 5, Fe—S subunit of oxidoreductase, flagellar M-ring protein, fumarate hydratase, gamma-glutamyltranspeptidase, gibberellin 20-oxidase, glucose dehydrogenase, glucose-1-phosphate cytidylyltransferase, glucose-6-phosphate 1-dehydrogenase, glutamate dehydrogenase, glutamate-ammonia-ligase, glutaredoxin, glycerate kinase, glycerol kinase, Glycine cleavage system T aminomethyltransferase, GM02LC17485-protein, GM02LC5744-protein, heat shock protein, heat shock transcription factor, hexose transporter, hydrolase, isochorismate synthase, isocitrate dehydrogenase, isocitrate lyase, leucyl/phenylalanyl-tRNA-protein transferase, lipopolysaccharide core biosynthesis protein, lipoprotein precursor, lysine decarboxylase, malate dehydrogenase, malonyl CoA-acyl carrier protein transacylase, membrane protein, membrane transport protein, methylglyoxal synthase, methylglyoxalase, methyltransferase, monothiol glutaredoxin, monthiol glutaredoxin, multiple antibiotic resistance protein, murein transglycosylase, NADH-quinone oxidoreductase subunit, O-antigen chain length determinant, oxidoreductase, oxidoreductase subunit, oxireductase, phenylacetic acid degradation protein, phenylalanine ammonia-lyase, phosphatase, phosphate import ATP-binding protein, photosystem I reaction centre subunit, photosystem II protein, polygalacturonase, protease, protein phosphatase, purine-nucleoside phosphorylase, pyruvate carboxylase, pyruvate kinase, recombinase A, ribulokinase, RNA polymerase sigma factor, sec-independent protein translocase, Sec-independent protein translocase subunit, slr1170-protein, thioredoxin, thioredoxin family protein, thioredoxin reductase, threonine efflux protein, threonine synthase, transcription factor, transcriptional regulator, transcriptional regulator protein, translation initiation factor subunit, TTC1386-protein, xanthine dehydrogenase, XM_{—}473199-protein, ycr102c-protein, YDL159W-A-protein, ygl237c-protein, YGR193C-protein, ygr221 c-protein, yhr131c-protein, yhr207c-protein, YKL038W-protein, YKL140W-protein, ylr065c-protein, ylr326w-protein, yml084w-protein, yol073c-protein, yol160w-protein, zinc finger protein, and zinc transporter, for example of the respective polypeptide as depicted in Table II, application no. 15, column 5 and 8, or a homolog or a fragment thereof, or the respective polypeptide comprising a sequence corresponding to the consensus sequences as shown in Table IV, application no. 15, column 8, or the respective polypeptide comprising at least one polypeptide motif as depicted in Table IV, application no. 15, column 8.

A further embodiment of the present invention relates to a process for the production of succinate, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of malate, which comprises
(a) increasing or generating the activity of a At1g13930-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a At1g13930-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of malate, which comprises
(a) increasing or generating the activity of a eukaryotic translation initiation factor 5 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of vitamin c and/or threonic acid, which comprises
(a) increasing or generating the activity of a eukaryotic translation initiation factor 5 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of fumarate, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of fumarate, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of vitamin c and/or threonic acid, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of fumarate, which comprises
(a) increasing or generating the activity of a CBL-interacting protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a CBL-interacting protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a CBL-interacting protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating the activity of a phenylalanine ammonia-lyase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating the activity of a thioredoxin reductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of succinate, which comprises
(a) increasing or generating the activity of a citrate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citramalate, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of succinate, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of malate, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating the activity of a thioredoxin family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a thioredoxin family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of glycerate, which comprises
(a) increasing or generating the activity of a thioredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

A further embodiment of the present invention relates to a process for the production of fumarate, which comprises
(a) increasing or generating the activity of a auxin response factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a auxin response factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of citramalate, which comprises
(a) increasing or generating the activity of a monthiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating the activity of a monthiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of glycerate, which comprises
(a) increasing or generating the activity of a monthiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

A further embodiment of the present invention relates to a process for the production of malate, which comprises
(a) increasing or generating the activity of a monthiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a monthiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of vitamin c and/or threonic acid, which comprises
(a) increasing or generating the activity of a monthiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of citramalate, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of glycerate, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

A further embodiment of the present invention relates to a process for the production of malate, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of glycerate, which comprises
(a) increasing or generating the activity of a thioredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

A further embodiment of the present invention relates to a process for the production of citramalate, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of glycerate, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

A further embodiment of the present invention relates to a process for the production of malate, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of vitamin c and/or threonic acid, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of fumarate, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of glycerate, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

A further embodiment of the present invention relates to a process for the production of malate, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citramalate, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of vitamin c and/or threonic acid, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of glycerate, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

A further embodiment of the present invention relates to a process for the production of citramalate, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of succinate, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citramalate, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of vitamin c and/or threonic acid, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of malate, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of fumarate, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of glycerate, which comprises
(a) increasing or generating the activity of a gibberellin 20-oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

A further embodiment of the present invention relates to a process for the production of malate, which comprises
(a) increasing or generating the activity of a gibberellin 20-oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of malate, which comprises
(a) increasing or generating the activity of a translation initiation factor subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of fumarate, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of succinate, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating the activity of a zinc finger protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of citramalate, which comprises
(a) increasing or generating the activity of a CDP-diacylglycerol-serine O-phosphatidyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating the activity of a CDP-diacylglycerol-serine O-phosphatidyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a CDP-diacylglycerol-serine O-phosphatidyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of malate, which comprises
(a) increasing or generating the activity of a Glycine cleavage system T aminomethyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of malate, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of malate, which comprises
(a) increasing or generating the activity of a adenylylsulfate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a adenylylsulfate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating the activity of a sec-independent protein translocase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a sec-independent protein translocase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of fumarate, which comprises
(a) increasing or generating the activity of a fumarate hydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of succinate, which comprises
(a) increasing or generating the activity of a transcriptional regulator protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of glycerate, which comprises
(a) increasing or generating the activity of a beta-hydroxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

A further embodiment of the present invention relates to a process for the production of succinate, which comprises
(a) increasing or generating the activity of a 30S ribosomal protein S3 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citramalate, which comprises
(a) increasing or generating the activity of a 50S ribosomal protein L14 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of malate, which comprises
(a) increasing or generating the activity of a acyl-CoA synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of citramalate, which comprises
(a) increasing or generating the activity of a 2-oxoglutarate dehydrogenase E1 subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating the activity of a 2-oxoglutarate dehydrogenase E1 subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a 2-oxoglutarate dehydrogenase E1 subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a 2-oxoglutarate dehydrogenase E1 subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of vitamin c and/or threonic acid, which comprises
(a) increasing or generating the activity of a 2-oxoglutarate dehydrogenase E1 subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of citramalate, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating the activity of a CTP synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a CTP synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a CTP synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of succinate, which comprises
(a) increasing or generating the activity of a recombinase A non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of malate, which comprises
(a) increasing or generating the activity of a glucose-1-phosphate cytidylyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of fumarate, which comprises
(a) increasing or generating the activity of a CDP-glucose-4,6-dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a CDP-glucose-4,6-dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of malate, which comprises
(a) increasing or generating the activity of a aminotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a isocitrate lyase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating the activity of a NADH-quinone oxidoreductase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a NADH-quinone oxidoreductase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of malate, which comprises
(a) increasing or generating the activity of a RNA polymerase sigma factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of malate, which comprises
(a) increasing or generating the activity of a hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of malate, which comprises
(a) increasing or generating the activity of a glutamate-ammonia-ligase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a electron transfer flavoprotein subunit beta non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating the activity of a AX653549-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a AX653549-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of fumarate, which comprises
(a) increasing or generating the activity of a AY087308-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a AY087308-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a AY087308-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating the activity of a fumarate hydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a threonine synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of fumarate, which comprises
(a) increasing or generating the activity of a ribulokinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating the activity of a ribulokinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citrate, which comprises
(a) increasing or generating the activity of a glucose dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrate or a composition comprising citrate in said non-human organism or in the culture medium surrounding said non-human organism.

A further embodiment of the present invention relates to a process for the production of fumarate, which comprises
(a) increasing or generating the activity of a acyl-CoA dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of citrate, which comprises
(a) increasing or generating the activity of a betaine aldehyde dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrate or a composition comprising citrate in said non-human organism or in the culture medium surrounding said non-human organism.

A further embodiment of the present invention relates to a process for the production of malate, which comprises
(a) increasing or generating the activity of a ATP-binding component of a transport system non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of malate, which comprises
(a) increasing or generating the activity of a membrane transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a membrane transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citrate, which comprises
(a) increasing or generating the activity of a glycerate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrate or a composition comprising citrate in said non-human organism or in the culture medium surrounding said non-human organism.

A further embodiment of the present invention relates to a process for the production of fumarate, which comprises
(a) increasing or generating the activity of a isochorismate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a isochorismate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of fumarate, which comprises
(a) increasing or generating the activity of a zinc transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating the activity of a b0801-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of malate, which comprises
(a) increasing or generating the activity of a leucyl/phenylalanyl-tRNA-protein transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of vitamin c and/or threonic acid, which comprises
(a) increasing or generating the activity of a methylglyoxal synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of malate, which comprises
(a) increasing or generating the activity of a b1003-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of malate, which comprises
(a) increasing or generating the activity of a membrane protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a b1285-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of malate, which comprises
(a) increasing or generating the activity of a phenylacetic acid degradation protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of malate, which comprises
(a) increasing or generating the activity of a b1425-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a lipoprotein precursor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citramalate, which comprises
(a) increasing or generating the activity of a b1445-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of citramalate, which comprises
(a) increasing or generating the activity of a b1522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of malate, which comprises
(a) increasing or generating the activity of a b1522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of fumarate, which comprises
(a) increasing or generating the activity of a Fe—S subunit of oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of fumarate, which comprises
(a) increasing or generating the activity of a fumarate hydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating the activity of a electron transport complex protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of succinate, which comprises
(a) increasing or generating the activity of a methylglyoxalase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citramalate, which comprises
(a) increasing or generating the activity of a b1670-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of malate, which comprises
(a) increasing or generating the activity of a b1670-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of citramalate, which comprises
(a) increasing or generating the activity of a b1672-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of malate, which comprises
(a) increasing or generating the activity of a b1672-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of vitamin c and/or threonic acid, which comprises
(a) increasing or generating the activity of a b1672-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of malate, which comprises
(a) increasing or generating the activity of a b1742-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a b1742-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of succinate, which comprises
(a) increasing or generating the activity of a arginine N-succinyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of vitamin c and/or threonic acid, which comprises
(a) increasing or generating the activity of a ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of malate, which comprises
(a) increasing or generating the activity of a protease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of vitamin c and/or threonic acid, which comprises
(a) increasing or generating the activity of a dATP pyrophosphohydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of succinate, which comprises
(a) increasing or generating the activity of a b1898-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating the activity of a flagellar M-ring protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a flagellar M-ring protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a flagellar M-ring protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of malate, which comprises
(a) increasing or generating the activity of a O-antigen chain length determinant non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of fumarate, which comprises
(a) increasing or generating the activity of a b2399-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating the activity of a b2399-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of vitamin c and/or threonic acid, which comprises
(a) increasing or generating the activity of a b2399-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of vitamin c and/or threonic acid, which comprises
(a) increasing or generating the activity of a cysteine synthase A non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of vitamin c and/or threonic acid, which comprises
(a) increasing or generating the activity of a cysteine synthase B non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of fumarate, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a murein transglycosylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of succinate, which comprises
(a) increasing or generating the activity of a 2-C-methyl-D-erythritol 4-phosphate cytidylyltransferase non-targeted in a non-human organism or a part thereof, preferably a microorganism organism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of succinate, which comprises
(a) increasing or generating the activity of a amino-acid acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of succinate, which comprises
(a) increasing or generating the activity of a xanthine dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of succinate, which comprises
(a) increasing or generating the activity of a cystathionine beta-lyase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating the activity of a methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of fumarate, which comprises
(a) increasing or generating the activity of a lipopolysaccharide core biosynthesis protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating the activity of a b3777-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of malate, which comprises
(a) increasing or generating the activity of a b3791-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a b3791-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citramalate, which comprises
(a) increasing or generating the activity of a b3814-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating the activity of a b3817-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of citramalate, which comprises
(a) increasing or generating the activity of a threonine efflux protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of malate, which comprises
(a) increasing or generating the activity of a threonine efflux protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a b3989-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of fumarate, which comprises
(a) increasing or generating the activity of a b4029-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a b4029-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a b4029-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating the activity of a b4050-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a b4050-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of citramalate, which comprises
(a) increasing or generating the activity of a b4056-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of citrate, which comprises
(a) increasing or generating the activity of a aspartase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrate or a composition comprising citrate in said non-human organism or in the culture medium surrounding said non-human organism.

A further embodiment of the present invention relates to a process for the production of fumarate, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of vitamin c and/or threonic acid, which comprises
(a) increasing or generating the activity of a purine-nucleoside phosphorylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of succinate, which comprises
(a) increasing or generating the activity of a lysine decarboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of succinate, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating the activity of a GM02LC17485-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of citramalate, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of citramalate, which comprises
(a) increasing or generating the activity of a GM02LC5744-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating the activity of a GM02LC5744-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of citramalate, which comprises
(a) increasing or generating the activity of a heat shock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating the activity of a agmatinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a ABC transporter ATP binding component non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a ABC transporter ATP binding component non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating the activity of a oxireductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating the activity of a phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citramalate, which comprises
(a) increasing or generating the activity of a CDP-diacylglycerol-glycerol-3-phosphate 3-phosphatidyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of malate, which comprises
(a) increasing or generating the activity of a CDP-diacylglycerol-glycerol-3-phosphate 3-phosphatidyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a adenylate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citramalate, which comprises
(a) increasing or generating the activity of a cation-transporting ATPase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of malate, which comprises
(a) increasing or generating the activity of a cation-transporting ATPase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of fumarate, which comprises
(a) increasing or generating the activity of a fumarate hydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of citramalate, which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating the activity of a bifunctional purine biosynthesis protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of citramalate, which comprises
(a) increasing or generating the activity of a malate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of succinate, which comprises
(a) increasing or generating the activity of a malate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of glycerate, which comprises
(a) increasing or generating the activity of a dihydrolipoamide dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

A further embodiment of the present invention relates to a process for the production of succinate, which comprises
(a) increasing or generating the activity of a dihydrolipoamide dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of succinate, which comprises
(a) increasing or generating the activity of a slr1170-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating the activity of a phosphate import ATP-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating the activity of a gamma-glutamyltranspeptidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of malate, which comprises
(a) increasing or generating the activity of a photosystem II protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of fumarate, which comprises
(a) increasing or generating the activity of a malonyl CoA-acyl carrier protein transacylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating the activity of a malonyl CoA-acyl carrier protein transacylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of glycerate, which comprises
(a) increasing or generating the activity of a photosystem I reaction centre subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

A further embodiment of the present invention relates to a process for the production of fumarate, which comprises
(a) increasing or generating the activity of a Sec-independent protein translocase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating the activity of a Sec-independent protein translocase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citramalate, which comprises
(a) increasing or generating the activity of a 2-isopropylmalate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating the activity of a multiple antibiotic resistance protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating the activity of a multiple antibiotic resistance protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating the activity of a TTC1386-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a TTC1386-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a TTC1386-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citramalate, which comprises
(a) increasing or generating the activity of a oxidoreductase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of glycerate, which comprises
(a) increasing or generating the activity of a oxidoreductase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

A further embodiment of the present invention relates to a process for the production of malate, which comprises
(a) increasing or generating the activity of a oxidoreductase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of fumarate, which comprises
(a) increasing or generating the activity of a XM_{—}473199-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a XM_{—}473199-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a XM_{—}473199-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of succinate, which comprises
(a) increasing or generating the activity of a glutamate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citramalate, which comprises
(a) increasing or generating the activity of a acetyl-CoA hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of pyruvate, which comprises
(a) increasing or generating the activity of a acetyl-CoA hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of pyruvate or a composition comprising pyruvate in said non-human organism or in the culture medium surrounding said non-human organism.

A further embodiment of the present invention relates to a process for the production of malate, which comprises
(a) increasing or generating the activity of a pyruvate carboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of fumarate, which comprises
(a) increasing or generating the activity of a ycr102c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of fumarate, which comprises
(a) increasing or generating the activity of a YDL159W-A-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of fumarate, which comprises
(a) increasing or generating the activity of a coproporphyrinogen III oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating the activity of a coproporphyrinogen III oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of malate, which comprises
(a) increasing or generating the activity of a ygl237c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a ygl237c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating the activity of a YGR193C-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a YGR193C-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a YGR193C-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of malate, which comprises
(a) increasing or generating the activity of a ygr221c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of citrate, which comprises
(a) increasing or generating the activity of a glycerol kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrate or a composition comprising citrate in said non-human organism or in the culture medium surrounding said non-human organism.

A further embodiment of the present invention relates to a process for the production of malate, which comprises
(a) increasing or generating the activity of a yhr131c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a yhr207c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of glycerate, which comprises
(a) increasing or generating the activity of a DnaJ-like chaperone non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

A further embodiment of the present invention relates to a process for the production of oxalate, which comprises
(a) increasing or generating the activity of a hexose transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oxalate or a composition comprising oxalate in said non-human organism or in the culture medium surrounding said non-human organism.

A further embodiment of the present invention relates to a process for the production of citramalate, which comprises
(a) increasing or generating the activity of a branched chain aminotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating the activity of a polygalacturonase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a polygalacturonase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of malate, which comprises
(a) increasing or generating the activity of a YKL038W-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of vitamin c and/or threonic acid, which comprises
(a) increasing or generating the activity of a YKL140W-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of fumarate, which comprises
(a) increasing or generating the activity of a ylr065c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating the activity of a isocitrate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating the activity of a ylr326w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of glycerate, which comprises
(a) increasing or generating the activity of a ylr326w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

A further embodiment of the present invention relates to a process for the production of vitamin c and/or threonic acid, which comprises
(a) increasing or generating the activity of a ylr326w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of fumarate, which comprises
(a) increasing or generating the activity of a yml084w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating the activity of a yml084w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citramalate, which comprises
(a) increasing or generating the activity of a yol073c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of malate, which comprises
(a) increasing or generating the activity of a yol073c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of vitamin c and/or threonic acid, which comprises
(a) increasing or generating the activity of a yol160w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of malate, which comprises
(a) increasing or generating the activity of a acetyl-CoA acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of vitamin c and/or threonic acid, which comprises
(a) increasing or generating the activity of a ammonium transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of fumarate, which comprises
(a) increasing or generating the activity of a 47266012-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a 47266012-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a 47266012-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating the activity of a 49747384_SOYBEAN-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating the activity of a 49747384_SOYBEAN-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating the activity of a 49747384_SOYBEAN-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of vitamin c and/or threonic acid, which comprises
(a) increasing or generating the activity of a 49747384_SOYBEAN-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of citramalate, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of succinate, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citramalate, which comprises
(a) increasing or generating the activity of a glucose-6-phosphate 1-dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of citramalate, which comprises
(a) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical citramalate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical citramalate;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of citramalate, or a composition comprising citramalate in said non-human organism or in the culture medium surrounding said non-human organism.

A further embodiment of the present invention relates to a process for the production of citrate, which comprises
(a) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical citrate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical citrate;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of citrate, or a composition comprising citrate in said non-human organism or in the culture medium surrounding said non-human organism.

A further embodiment of the present invention relates to a process for the production of fumarate, which comprises
(a) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical fumarate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical fumarate;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of fumarate, or a composition comprising fumarate in said non-human organism or in the culture medium surrounding said non-human organism.

A further embodiment of the present invention relates to a process for the production of glycerate, which comprises
(a) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical glycerate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical glycerate;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of glycerate, or a composition comprising glycerate in said non-human organism or in the culture medium surrounding said non-human organism.

A further embodiment of the present invention relates to a process for the production of malate, which comprises
(a) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical malate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical malate;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of malate, or a composition comprising malate in said non-human organism or in the culture medium surrounding said non-human organism.

A further embodiment of the present invention relates to a process for the production of oxalate, which comprises
(a) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical oxalate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical oxalate;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of oxalate, or a composition comprising oxalate in said non-human organism or in the culture medium surrounding said non-human organism.

A further embodiment of the present invention relates to a process for the production of pyruvate, which comprises
(a) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical pyruvate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical pyruvate;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of pyruvate, or a composition comprising pyruvate in said non-human organism or in the culture medium surrounding said non-human organism.

A further embodiment of the present invention relates to a process for the production of succinate, which comprises
(a) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical succinate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical succinate;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of succinate, or a composition comprising succinate in said non-human organism or in the culture medium surrounding said non-human organism.

A further embodiment of the present invention relates to a process for the production of vitamin c and/or threonic acid, which comprises
(a) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical vitamin c and/or threonic acid; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical vitamin c and/or threonic acid;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of vitamin c and/or threonic acid, or a composition comprising vitamin c and/or threonic acid in said non-human organism or in the culture medium surrounding said non-human organism.

A further embodiment of the present invention relates to a process for the production of succinate, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of malate, which comprises
(a) increasing or generating one or more activities selected from the group consisting of At1g13930-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of At1g13930-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of malate, which comprises
(a) increasing or generating one or more activities selected from the group consisting of eukaryotic translation initiation factor 5 in an organelle, preferably in plastids or mitochondria, especially in plastids, of a non-human organism or a part thereof, preferably a microorganism organism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of vitamin c and/or threonic acid, which comprises
(a) increasing or generating one or more activities selected from the group consisting of eukaryotic translation initiation factor 5 in an organelle, preferably in plastids or mitochondria, especially in plastids, of a non-human organism or a part thereof, preferably a microorganism organism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of fumarate, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of fumarate, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of vitamin c and/or threonic acid, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of fumarate, which comprises
(a) increasing or generating one or more activities selected from the group consisting of CBL-interacting protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of CBL-interacting protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of CBL-interacting protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating one or more activities selected from the group consisting of phenylalanine ammonia-lyase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating one or more activities selected from the group consisting of thioredoxin reductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of succinate, which comprises
(a) increasing or generating one or more activities selected from the group consisting of citrate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citramalate, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of succinate, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of malate, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating one or more activities selected from the group consisting of thioredoxin family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of thioredoxin family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of glycerate, which comprises
(a) increasing or generating one or more activities selected from the group consisting of thioredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

A further embodiment of the present invention relates to a process for the production of fumarate, which comprises
(a) increasing or generating one or more activities selected from the group consisting of auxin response factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of auxin response factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of citramalate, which comprises
(a) increasing or generating one or more activities selected from the group consisting of monthiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating one or more activities selected from the group consisting of monthiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of glycerate, which comprises
(a) increasing or generating one or more activities selected from the group consisting of monthiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

A further embodiment of the present invention relates to a process for the production of malate, which comprises
(a) increasing or generating one or more activities selected from the group consisting of monthiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of monthiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of vitamin c and/or threonic acid, which comprises
(a) increasing or generating one or more activities selected from the group consisting of monthiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of citramalate, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of glycerate, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

A further embodiment of the present invention relates to a process for the production of malate, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of glycerate, which comprises
(a) increasing or generating one or more activities selected from the group consisting of thioredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

A further embodiment of the present invention relates to a process for the production of citramalate, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of glycerate, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

A further embodiment of the present invention relates to a process for the production of malate, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of vitamin c and/or threonic acid, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of fumarate, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of glycerate, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

A further embodiment of the present invention relates to a process for the production of malate, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citramalate, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of vitamin c and/or threonic acid, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of glycerate, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

A further embodiment of the present invention relates to a process for the production of citramalate, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of succinate, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citramalate, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of vitamin c and/or threonic acid, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of malate, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of fumarate, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of glycerate, which comprises
(a) increasing or generating one or more activities selected from the group consisting of gibberellin 20-oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

A further embodiment of the present invention relates to a process for the production of malate, which comprises
(a) increasing or generating one or more activities selected from the group consisting of gibberellin 20-oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of malate, which comprises
(a) increasing or generating one or more activities selected from the group consisting of translation initiation factor subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of fumarate, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of succinate, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating one or more activities selected from the group consisting of zinc finger protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of citramalate, which comprises
(a) increasing or generating one or more activities selected from the group consisting of CDP-diacylglycerol-serine O-phosphatidyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating one or more activities selected from the group consisting of CDP-diacylglycerol-serine O-phosphatidyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of CDP-diacylglycerol-serine O-phosphatidyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of malate, which comprises
(a) increasing or generating one or more activities selected from the group consisting of Glycine cleavage system T aminomethyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of malate, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of malate, which comprises
(a) increasing or generating one or more activities selected from the group consisting of adenylylsulfate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of adenylylsulfate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating one or more activities selected from the group consisting of sec-independent protein translocase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of sec-independent protein translocase in an organelle, preferably in plastids or mitochondria, especially in plastids, of a non-human organism or a part thereof, preferably a microorganism a plant cell, a plant or a part thereof, as compared to a corresponding non-transformed, wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of fumarate, which comprises
(a) increasing or generating one or more activities selected from the group consisting of fumarate hydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of succinate, which comprises
(a) increasing or generating one or more activities selected from the group consisting of transcriptional regulator protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of glycerate, which comprises
(a) increasing or generating one or more activities selected from the group consisting of beta-hydroxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

A further embodiment of the present invention relates to a process for the production of succinate, which comprises
(a) increasing or generating one or more activities selected from the group consisting of 30S ribosomal protein S3 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citramalate, which comprises
(a) increasing or generating one or more activities selected from the group consisting of 50S ribosomal protein L14 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of malate, which comprises
(a) increasing or generating one or more activities selected from the group consisting of acyl-CoA synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of citramalate, which comprises
(a) increasing or generating one or more activities selected from the group consisting of 2-oxoglutarate dehydrogenase E1 subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating one or more activities selected from the group consisting of 2-oxoglutarate dehydrogenase E1 subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of 2-oxoglutarate dehydrogenase E1 subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of 2-oxoglutarate dehydrogenase E1 subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of vitamin c and/or threonic acid, which comprises
(a) increasing or generating one or more activities selected from the group consisting of 2-oxoglutarate dehydrogenase E1 subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of citramalate, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating one or more activities selected from the group consisting of CTP synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of CTP synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of CTP synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of succinate, which comprises
(a) increasing or generating one or more activities selected from the group consisting of recombinase A in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of malate, which comprises
(a) increasing or generating one or more activities selected from the group consisting of glucose-1-phosphate cytidylyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of fumarate, which comprises
(a) increasing or generating one or more activities selected from the group consisting of CDP-glucose-4,6-dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of CDP-glucose-4,6-dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of malate, which comprises
(a) increasing or generating one or more activities selected from the group consisting of aminotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of isocitrate lyase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating one or more activities selected from the group consisting of NADH-quinone oxidoreductase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of NADH-quinone oxidoreductase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of malate, which comprises
(a) increasing or generating one or more activities selected from the group consisting of RNA polymerase sigma factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of malate, which comprises
(a) increasing or generating one or more activities selected from the group consisting of hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of malate, which comprises
(a) increasing or generating one or more activities selected from the group consisting of glutamate-ammonia-ligase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of electron transfer flavoprotein subunit beta in an organelle, preferably in plastids or mitochondria, especially in plastids, of a non-human organism or a part thereof, preferably a microorganism organism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating one or more activities selected from the group consisting of AX653549-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of AX653549-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of fumarate, which comprises
(a) increasing or generating one or more activities selected from the group consisting of AY087308-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of AY087308-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of AY087308-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating one or more activities selected from the group consisting of fumarate hydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of threonine synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of fumarate, which comprises
(a) increasing or generating one or more activities selected from the group consisting of ribulokinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating one or more activities selected from the group consisting of ribulokinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citrate, which comprises
(a) increasing or generating one or more activities selected from the group consisting of glucose dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrate or a composition comprising citrate in said non-human organism or in the culture medium surrounding said non-human organism.

A further embodiment of the present invention relates to a process for the production of fumarate, which comprises
(a) increasing or generating one or more activities selected from the group consisting of acyl-CoA dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of citrate, which comprises
(a) increasing or generating one or more activities selected from the group consisting of betaine aldehyde dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrate or a composition comprising citrate in said non-human organism or in the culture medium surrounding said non-human organism.

A further embodiment of the present invention relates to a process for the production of malate, which comprises
(a) increasing or generating one or more activities selected from the group consisting of ATP-binding component of a transport system in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of malate, which comprises
(a) increasing or generating one or more activities selected from the group consisting of membrane transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of membrane transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citrate, which comprises
(a) increasing or generating one or more activities selected from the group consisting of glycerate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrate or a composition comprising citrate in said non-human organism or in the culture medium surrounding said non-human organism.

A further embodiment of the present invention relates to a process for the production of fumarate, which comprises
(a) increasing or generating one or more activities selected from the group consisting of isochorismate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of isochorismate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of fumarate, which comprises
(a) increasing or generating one or more activities selected from the group consisting of zinc transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating one or more activities selected from the group consisting of b0801-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of malate, which comprises
(a) increasing or generating one or more activities selected from the group consisting of leucyl/phenylalanyl-tRNA-protein transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of vitamin c and/or threonic acid, which comprises
(a) increasing or generating one or more activities selected from the group consisting of methylglyoxal synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of malate, which comprises
(a) increasing or generating one or more activities selected from the group consisting of b1003-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of malate, which comprises
(a) increasing or generating one or more activities selected from the group consisting of membrane protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of b1285-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of malate, which comprises
(a) increasing or generating one or more activities selected from the group consisting of phenylacetic acid degradation protein in an organelle, preferably in plastids or mitochondria, especially in plastids, of a non-human organism or a part thereof, preferably a microorganism organism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of malate, which comprises
(a) increasing or generating one or more activities selected from the group consisting of b1425-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of lipoprotein precursor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citramalate, which comprises
(a) increasing or generating one or more activities selected from the group consisting of b1445-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of citramalate, which comprises
(a) increasing or generating one or more activities selected from the group consisting of b1522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of malate, which comprises
(a) increasing or generating one or more activities selected from the group consisting of b1522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of fumarate, which comprises
(a) increasing or generating one or more activities selected from the group consisting of Fe—S subunit of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of fumarate, which comprises
(a) increasing or generating one or more activities selected from the group consisting of fumarate hydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating one or more activities selected from the group consisting of electron transport complex protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of succinate, which comprises
(a) increasing or generating one or more activities selected from the group consisting of methylglyoxalase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citramalate, which comprises
(a) increasing or generating one or more activities selected from the group consisting of b1670-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of malate, which comprises
(a) increasing or generating one or more activities selected from the group consisting of b1670-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of citramalate, which comprises
(a) increasing or generating one or more activities selected from the group consisting of b1672-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of malate, which comprises
(a) increasing or generating one or more activities selected from the group consisting of b1672-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of vitamin c and/or threonic acid, which comprises
(a) increasing or generating one or more activities selected from the group consisting of b1672-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of malate, which comprises
(a) increasing or generating one or more activities selected from the group consisting of b1742-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of b1742-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of succinate, which comprises
(a) increasing or generating one or more activities selected from the group consisting of arginine N-succinyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of vitamin c and/or threonic acid, which comprises
(a) increasing or generating one or more activities selected from the group consisting of ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of malate, which comprises
(a) increasing or generating one or more activities selected from the group consisting of protease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of vitamin c and/or threonic acid, which comprises
(a) increasing or generating one or more activities selected from the group consisting of dATP pyrophosphohydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of succinate, which comprises
(a) increasing or generating one or more activities selected from the group consisting of b1898-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating one or more activities selected from the group consisting of flagellar M-ring protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of flagellar M-ring protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of flagellar M-ring protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of malate, which comprises
(a) increasing or generating one or more activities selected from the group consisting of O-antigen chain length determinant in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of fumarate, which comprises
(a) increasing or generating one or more activities selected from the group consisting of b2399-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating one or more activities selected from the group consisting of b2399-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of vitamin c and/or threonic acid, which comprises
(a) increasing or generating one or more activities selected from the group consisting of b2399-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of vitamin c and/or threonic acid, which comprises
(a) increasing or generating one or more activities selected from the group consisting of cysteine synthase A in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of vitamin c and/or threonic acid, which comprises
(a) increasing or generating one or more activities selected from the group consisting of cysteine synthase B in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of fumarate, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of murein transglycosylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of succinate, which comprises
(a) increasing or generating one or more activities selected from the group consisting of 2-C-methyl-D-erythritol 4-phosphate cytidylyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of succinate, which comprises
(a) increasing or generating one or more activities selected from the group consisting of amino-acid acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of succinate, which comprises
(a) increasing or generating one or more activities selected from the group consisting of xanthine dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of succinate, which comprises
(a) increasing or generating one or more activities selected from the group consisting of cystathionine beta-lyase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating one or more activities selected from the group consisting of methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of fumarate, which comprises
(a) increasing or generating one or more activities selected from the group consisting of lipopolysaccharide core biosynthesis protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating one or more activities selected from the group consisting of b3777-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of malate, which comprises
(a) increasing or generating one or more activities selected from the group consisting of b3791-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of b3791-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citramalate, which comprises
(a) increasing or generating one or more activities selected from the group consisting of b3814-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating one or more activities selected from the group consisting of b3817-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of citramalate, which comprises
(a) increasing or generating one or more activities selected from the group consisting of threonine efflux protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of malate, which comprises
(a) increasing or generating one or more activities selected from the group consisting of threonine efflux protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of b3989-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of fumarate, which comprises
(a) increasing or generating one or more activities selected from the group consisting of b4029-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of b4029-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of b4029-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating one or more activities selected from the group consisting of b4050-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of b4050-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of citramalate, which comprises
(a) increasing or generating one or more activities selected from the group consisting of b4056-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of citrate, which comprises
(a) increasing or generating one or more activities selected from the group consisting of aspartase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrate or a composition comprising citrate in said non-human organism or in the culture medium surrounding said non-human organism.

A further embodiment of the present invention relates to a process for the production of fumarate, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of vitamin c and/or threonic acid, which comprises
(a) increasing or generating one or more activities selected from the group consisting of purine-nucleoside phosphorylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of succinate, which comprises
(a) increasing or generating one or more activities selected from the group consisting of lysine decarboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of succinate, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating one or more activities selected from the group consisting of GM02LC17485-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of citramalate, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of citramalate, which comprises
(a) increasing or generating one or more activities selected from the group consisting of GM02LC5744-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating one or more activities selected from the group consisting of GM02LC5744-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of citramalate, which comprises
(a) increasing or generating one or more activities selected from the group consisting of heat shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating one or more activities selected from the group consisting of agmatinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of ABC transporter ATP binding component in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of ABC transporter ATP binding component in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating one or more activities selected from the group consisting of oxireductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating one or more activities selected from the group consisting of phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citramalate, which comprises
(a) increasing or generating one or more activities selected from the group consisting of CDP-diacylglycerol-glycerol-3-phosphate 3-phosphatidyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of malate, which comprises
(a) increasing or generating one or more activities selected from the group consisting of CDP-diacylglycerol-glycerol-3-phosphate 3-phosphatidyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of adenylate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citramalate, which comprises
(a) increasing or generating one or more activities selected from the group consisting of cation-transporting ATPase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of malate, which comprises
(a) increasing or generating one or more activities selected from the group consisting of cation-transporting ATPase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of fumarate, which comprises
(a) increasing or generating one or more activities selected from the group consisting of fumarate hydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of citramalate, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating one or more activities selected from the group consisting of bifunctional purine biosynthesis protein in an organelle, preferably in plastids or mitochondria, especially in plastids, of a non-human organism or a part thereof, preferably a microorganism organism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of citramalate, which comprises
(a) increasing or generating one or more activities selected from the group consisting of malate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of succinate, which comprises
(a) increasing or generating one or more activities selected from the group consisting of malate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of glycerate, which comprises
(a) increasing or generating one or more activities selected from the group consisting of dihydrolipoamide dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

A further embodiment of the present invention relates to a process for the production of succinate, which comprises
(a) increasing or generating one or more activities selected from the group consisting of dihydrolipoamide dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of succinate, which comprises
(a) increasing or generating one or more activities selected from the group consisting of slr1170-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating one or more activities selected from the group consisting of phosphate import ATP-binding protein in an organelle, preferably in plastids or mitochondria, especially in plastids, of a non-human organism or a part thereof, preferably a microorganism organism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating one or more activities selected from the group consisting of gamma-glutamyltranspeptidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of malate, which comprises
(a) increasing or generating one or more activities selected from the group consisting of photosystem II protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of fumarate, which comprises
(a) increasing or generating one or more activities selected from the group consisting of malonyl CoA-acyl carrier protein transacylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating one or more activities selected from the group consisting of malonyl CoA-acyl carrier protein transacylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of glycerate, which comprises
(a) increasing or generating one or more activities selected from the group consisting of photosystem I reaction centre subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

A further embodiment of the present invention relates to a process for the production of fumarate, which comprises
(a) increasing or generating one or more activities selected from the group consisting of Secindependent protein translocase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating one or more activities selected from the group consisting of Sec-independent protein translocase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citramalate, which comprises
(a) increasing or generating one or more activities selected from the group consisting of 2-isopropylmalate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating one or more activities selected from the group consisting of multiple antibiotic resistance protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating one or more activities selected from the group consisting of multiple antibiotic resistance protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating one or more activities selected from the group consisting of TTC1386-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of TTC1386-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of TTC1386-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citramalate, which comprises
(a) increasing or generating one or more activities selected from the group consisting of oxidoreductase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of glycerate, which comprises
(a) increasing or generating one or more activities selected from the group consisting of oxidoreductase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

A further embodiment of the present invention relates to a process for the production of malate, which comprises
(a) increasing or generating one or more activities selected from the group consisting of oxidoreductase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of fumarate, which comprises
(a) increasing or generating one or more activities selected from the group consisting of XM_{—}473199-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of XM_{—}473199-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of XM_{—}473199-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of succinate, which comprises
(a) increasing or generating one or more activities selected from the group consisting of glutamate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citramalate, which comprises
(a) increasing or generating one or more activities selected from the group consisting of acetyl-CoA hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of pyruvate, which comprises
(a) increasing or generating one or more activities selected from the group consisting of acetyl-CoA hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of pyruvate or a composition comprising pyruvate in said non-human organism or in the culture medium surrounding said non-human organism.

A further embodiment of the present invention relates to a process for the production of malate, which comprises
(a) increasing or generating one or more activities selected from the group consisting of pyruvate carboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of fumarate, which comprises
(a) increasing or generating one or more activities selected from the group consisting of ycr102c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of fumarate, which comprises
(a) increasing or generating one or more activities selected from the group consisting of YDL159W-A-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of fumarate, which comprises
(a) increasing or generating one or more activities selected from the group consisting of coproporphyrinogen III oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating one or more activities selected from the group consisting of coproporphyrinogen III oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of malate, which comprises
(a) increasing or generating one or more activities selected from the group consisting of ygl237c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of ygl237c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating one or more activities selected from the group consisting of YGR193C-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of YGR193C-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of YGR193C-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of malate, which comprises
(a) increasing or generating one or more activities selected from the group consisting of ygr221c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of citrate, which comprises
(a) increasing or generating one or more activities selected from the group consisting of glycerol kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrate or a composition comprising citrate in said non-human organism or in the culture medium surrounding said non-human organism.

A further embodiment of the present invention relates to a process for the production of malate, which comprises
(a) increasing or generating one or more activities selected from the group consisting of yhr131c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of yhr207c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of glycerate, which comprises
(a) increasing or generating one or more activities selected from the group consisting of DnaJ-like chaperone in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

A further embodiment of the present invention relates to a process for the production of oxalate, which comprises
(a) increasing or generating one or more activities selected from the group consisting of hexose transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oxalate or a composition comprising oxalate in said non-human organism or in the culture medium surrounding said non-human organism.

A further embodiment of the present invention relates to a process for the production of citramalate, which comprises
(a) increasing or generating one or more activities selected from the group consisting of branched chain aminotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of fumarate, which comprises
(a) increasing or generating one or more activities selected from the group consisting of polygalacturonase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of polygalacturonase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of malate, which comprises
(a) increasing or generating one or more activities selected from the group consisting of YKL038W-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of vitamin c and/or threonic acid, which comprises
(a) increasing or generating one or more activities selected from the group consisting of YKL140W-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of fumarate, which comprises
(a) increasing or generating one or more activities selected from the group consisting of ylr065c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating one or more activities selected from the group consisting of isocitrate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating one or more activities selected from the group consisting of ylr326w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of glycerate, which comprises
(a) increasing or generating one or more activities selected from the group consisting of ylr326w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

A further embodiment of the present invention relates to a process for the production of vitamin c and/or threonic acid, which comprises
(a) increasing or generating one or more activities selected from the group consisting of ylr326w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of fumarate, which comprises
(a) increasing or generating one or more activities selected from the group consisting of yml084w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of succinate, which comprises
(a) increasing or generating one or more activities selected from the group consisting of yml084w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citramalate, which comprises
(a) increasing or generating one or more activities selected from the group consisting of yol073c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of malate, which comprises
(a) increasing or generating one or more activities selected from the group consisting of yol073c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of vitamin c and/or threonic acid, which comprises
(a) increasing or generating one or more activities selected from the group consisting of yol160w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of malate, which comprises
(a) increasing or generating one or more activities selected from the group consisting of acetyl-CoA acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of vitamin c and/or threonic acid, which comprises
(a) increasing or generating one or more activities selected from the group consisting of ammonium transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of fumarate, which comprises
(a) increasing or generating one or more activities selected from the group consisting of 47266012-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of 47266012-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of 47266012-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of fumarate, which comprises
(a) increasing or generating one or more activities selected from the group consisting of 49747384_SOYBEAN-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

A further embodiment of the present invention relates to a process for the production of malate, which comprises
(a) increasing or generating one or more activities selected from the group consisting of 49747384_SOYBEAN-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

A further embodiment of the present invention relates to a process for the production of succinate, which comprises
(a) increasing or generating one or more activities selected from the group consisting of 49747384_SOYBEAN-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of vitamin c and/or threonic acid, which comprises
(a) increasing or generating one or more activities selected from the group consisting of 49747384_SOYBEAN-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

A further embodiment of the present invention relates to a process for the production of citramalate, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of succinate, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

A further embodiment of the present invention relates to a process for the production of citramalate, which comprises
(a) increasing or generating one or more activities selected from the group consisting of glucose-6-phosphate 1-dehydrogenase in an organelle, preferably in plastids or mitochondria, especially in plastids, of a non-human organism or a part thereof, preferably a microorganism organism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

A further embodiment of the present invention relates to a process for the production of citramalate, which comprises
(a1) increasing or generating the activity of a polypeptide comprising a polypeptide as depicted in the respective line in column 5 or 8 of Table II, application no. 15, or a homolog or fragment thereof, a consensus sequence or at least one polypeptide motif as depicted in column 8 of Table IV, application no. 15, whereby the respective line disclose in column 7 the fine chemical citramalate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical citramalate;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, which is joined to a transit peptide, whereby the respective line disclose in column 7 the fine chemical citramalate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical citramalate;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical citramalate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical citramalate;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of citramalate, or a composition comprising citramalate in said non-human organism or in the culture medium surrounding said non-human organism.

A further embodiment of the present invention relates to a process for the production of citrate, which comprises
(a1) increasing or generating the activity of a polypeptide comprising a polypeptide as depicted in the respective line in column 5 or 8 of Table II, application no. 15, or a homolog or fragment thereof, a consensus sequence or at least one polypeptide motif as depicted in column 8 of Table IV, application no. 15, whereby the respective line disclose in column 7 the fine chemical citrate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical citrate;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, which is joined to a transit peptide, whereby the respective line disclose in column 7 the fine chemical citrate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical citrate;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical citrate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical citrate;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of citrate, or a composition comprising citrate in said non-human organism or in the culture medium surrounding said non-human organism.

A further embodiment of the present invention relates to a process for the production of fumarate, which comprises
(a1) increasing or generating the activity of a polypeptide comprising a polypeptide as depicted in the respective line in column 5 or 8 of Table II, application no. 15, or a homolog or fragment thereof, a consensus sequence or at least one polypeptide motif as depicted in column 8 of Table IV, application no. 15, whereby the respective line disclose in column 7 the fine chemical fumarate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical fumarate;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, which is joined to a transit peptide, whereby the respective line disclose in column 7 the fine chemical fumarate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical fumarate;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical fumarate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical fumarate;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of fumarate, or a composition comprising fumarate in said non-human organism or in the culture medium surrounding said non-human organism.

A further embodiment of the present invention relates to a process for the production of glycerate, which comprises
(a1) increasing or generating the activity of a polypeptide comprising a polypeptide as depicted in the respective line in column 5 or 8 of Table II, application no. 15, or a homolog or fragment thereof, a consensus sequence or at least one polypeptide motif as depicted in column 8 of Table IV, application no. 15, whereby the respective line disclose in column 7 the fine chemical glycerate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical glycerate;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, which is joined to a transit peptide, whereby the respective line disclose in column 7 the fine chemical glycerate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical glycerate;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical glycerate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical glycerate;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of glycerate, or a composition comprising glycerate in said non-human organism or in the culture medium surrounding said non-human organism.

A further embodiment of the present invention relates to a process for the production of malate, which comprises
(a1) increasing or generating the activity of a polypeptide comprising a polypeptide as depicted in the respective line in column 5 or 8 of Table II, application no. 15, or a homolog or fragment thereof, a consensus sequence or at least one polypeptide motif as depicted in column 8 of Table IV, application no. 15, whereby the respective line disclose in column 7 the fine chemical malate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical malate;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, which is joined to a transit peptide, whereby the respective line disclose in column 7 the fine chemical malate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical malate;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical malate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical malate;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of malate, or a composition comprising malate in said non-human organism or in the culture medium surrounding said non-human organism.

A further embodiment of the present invention relates to a process for the production of oxalate, which comprises
(a1) increasing or generating the activity of a polypeptide comprising a polypeptide as depicted in the respective line in column 5 or 8 of Table II, application no. 15, or a homolog or fragment thereof, a consensus sequence or at least one polypeptide motif as depicted in column 8 of Table IV, application no. 15, whereby the respective line disclose in column 7 the fine chemical oxalate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical oxalate;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, which is joined to a transit peptide, whereby the respective line disclose in column 7 the fine chemical oxalate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical oxalate;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical oxalate; or increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical oxalate;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of oxalate, or a composition comprising oxalate in said non-human organism or in the culture medium surrounding said non-human organism.

A further embodiment of the present invention relates to a process for the production of pyruvate, which comprises
(a1) increasing or generating the activity of a polypeptide comprising a polypeptide as depicted in the respective line in column 5 or 8 of Table II, application no. 15, or a homolog or fragment thereof, a consensus sequence or at least one polypeptide motif as depicted in column 8 of Table IV, application no. 15, whereby the respective line disclose in column 7 the fine chemical pyruvate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical pyruvate;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, which is joined to a transit peptide, whereby the respective line disclose in column 7 the fine chemical pyruvate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical pyruvate;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical pyruvate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical pyruvate;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of pyruvate, or a composition comprising pyruvate in said non-human organism or in the culture medium surrounding said non-human organism.

A further embodiment of the present invention relates to a process for the production of succinate, which comprises
(a1) increasing or generating the activity of a polypeptide comprising a polypeptide as depicted in the respective line in column 5 or 8 of Table II, application no. 15, or a homolog or fragment thereof, a consensus sequence or at least one polypeptide motif as depicted in column 8 of Table IV, application no. 15, whereby the respective line disclose in column 7 the fine chemical succinate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical succinate;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, which is joined to a transit peptide, whereby the respective line disclose in column 7 the fine chemical succinate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical succinate;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical succinate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical succinate;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of succinate, or a composition comprising succinate in said non-human organism or in the culture medium surrounding said non-human organism.

A further embodiment of the present invention relates to a process for the production of vitamin c and/or threonic acid, which comprises
(a1) increasing or generating the activity of a polypeptide comprising a polypeptide as depicted in the respective line in column 5 or 8 of Table II, application no. 15, or a homolog or fragment thereof, a consensus sequence or at least one polypeptide motif as depicted in column 8 of Table IV, application no. 15, whereby the respective line disclose in column 7 the fine chemical vitamin c and/or threonic acid; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical vitamin c and/or threonic acid;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, which is joined to a transit peptide, whereby the respective line disclose in column 7 the fine chemical vitamin c and/or threonic acid; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical vitamin c and/or threonic acid;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical vitamin c and/or threonic acid; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical vitamin c and/or threonic acid;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of vitamin c and/or threonic acid, or a composition comprising vitamin c and/or threonic acid in said non-human organism or in the culture medium surrounding said non-human organism.

Accordingly, the present invention relates to a process for the production of succinate, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of malate, which comprises
(a) increasing or generating the activity of a At1g13930-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a At1g13930-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of malate, which comprises
(a) increasing or generating the activity of a eukaryotic translation initiation factor 5 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of vitamin c and/or threonic acid, which comprises
(a) increasing or generating the activity of a eukaryotic translation initiation factor 5 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of fumarate, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of fumarate, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of vitamin c and/or threonic acid, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of fumarate, which comprises
(a) increasing or generating the activity of a CBL-interacting protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a CBL-interacting protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a CBL-interacting protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating the activity of a phenylalanine ammonia-lyase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating the activity of a thioredoxin reductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of succinate, which comprises
(a) increasing or generating the activity of a citrate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citramalate, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of succinate, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of malate, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating the activity of a thioredoxin family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a thioredoxin family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of glycerate, which comprises
(a) increasing or generating the activity of a thioredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

Accordingly, the present invention relates to a process for the production of fumarate, which comprises
(a) increasing or generating the activity of a auxin response factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a auxin response factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of citramalate, which comprises
(a) increasing or generating the activity of a monthiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating the activity of a monthiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of glycerate, which comprises
(a) increasing or generating the activity of a monthiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

Accordingly, the present invention relates to a process for the production of malate, which comprises
(a) increasing or generating the activity of a monthiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a monthiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of vitamin c and/or threonic acid, which comprises
(a) increasing or generating the activity of a monthiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of citramalate, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of glycerate, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

Accordingly, the present invention relates to a process for the production of malate, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of glycerate, which comprises
(a) increasing or generating the activity of a thioredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

Accordingly, the present invention relates to a process for the production of citramalate, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of glycerate, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

Accordingly, the present invention relates to a process for the production of malate, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of vitamin c and/or threonic acid, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of fumarate, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of glycerate, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

Accordingly, the present invention relates to a process for the production of malate, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citramalate, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of vitamin c and/or threonic acid, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of glycerate, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

Accordingly, the present invention relates to a process for the production of citramalate, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of succinate, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citramalate, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of vitamin c and/or threonic acid, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of malate, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of fumarate, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of glycerate, which comprises
(a) increasing or generating the activity of a gibberellin 20-oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

Accordingly, the present invention relates to a process for the production of malate, which comprises
(a) increasing or generating the activity of a gibberellin 20-oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of malate, which comprises
(a) increasing or generating the activity of a translation initiation factor subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of fumarate, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of succinate, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating the activity of a zinc finger protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of citramalate, which comprises
(a) increasing or generating the activity of a CDP-diacylglycerol-serine O-phosphatidyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating the activity of a CDP-diacylglycerol-serine O-phosphatidyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a CDP-diacylglycerol-serine O-phosphatidyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of malate, which comprises
(a) increasing or generating the activity of a Glycine cleavage system T aminomethyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of malate, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of malate, which comprises
(a) increasing or generating the activity of a adenylylsulfate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a adenylylsulfate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating the activity of a sec-independent protein translocase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a sec-independent protein translocase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of fumarate, which comprises
(a) increasing or generating the activity of a fumarate hydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of succinate, which comprises
(a) increasing or generating the activity of a transcriptional regulator protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of glycerate, which comprises
(a) increasing or generating the activity of a beta-hydroxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

Accordingly, the present invention relates to a process for the production of succinate, which comprises
(a) increasing or generating the activity of a 30S ribosomal protein S3 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citramalate, which comprises
(a) increasing or generating the activity of a 50S ribosomal protein L14 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of malate, which comprises
(a) increasing or generating the activity of a acyl-CoA synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of citramalate, which comprises
(a) increasing or generating the activity of a 2-oxoglutarate dehydrogenase E1 subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating the activity of a 2-oxoglutarate dehydrogenase E1 subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a 2-oxoglutarate dehydrogenase E1 subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a 2-oxoglutarate dehydrogenase E1 subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of vitamin c and/or threonic acid, which comprises
(a) increasing or generating the activity of a 2-oxoglutarate dehydrogenase E1 subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of citramalate, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating the activity of a CTP synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a CTP synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a CTP synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of succinate, which comprises
(a) increasing or generating the activity of a recombinase A in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of malate, which comprises
(a) increasing or generating the activity of a glucose-1-phosphate cytidylyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of fumarate, which comprises
(a) increasing or generating the activity of a CDP-glucose-4,6-dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a CDP-glucose-4,6-dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of malate, which comprises
(a) increasing or generating the activity of a aminotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a isocitrate lyase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating the activity of a NADH-quinone oxidoreductase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a NADH-quinone oxidoreductase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of malate, which comprises
(a) increasing or generating the activity of a RNA polymerase sigma factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of malate, which comprises
(a) increasing or generating the activity of a hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of malate, which comprises
(a) increasing or generating the activity of a glutamate-ammonia-ligase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a electron transfer flavoprotein subunit beta in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating the activity of a AX653549-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a AX653549-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of fumarate, which comprises
(a) increasing or generating the activity of a AY087308-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a AY087308-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a AY087308-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating the activity of a fumarate hydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a threonine synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of fumarate, which comprises
(a) increasing or generating the activity of a ribulokinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating the activity of a ribulokinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citrate, which comprises
(a) increasing or generating the activity of a glucose dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrate or a composition comprising citrate in said non-human organism or in the culture medium surrounding said non-human organism.

Accordingly, the present invention relates to a process for the production of fumarate, which comprises
(a) increasing or generating the activity of a acyl-CoA dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of citrate, which comprises
(a) increasing or generating the activity of a betaine aldehyde dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrate or a composition comprising citrate in said non-human organism or in the culture medium surrounding said non-human organism.

Accordingly, the present invention relates to a process for the production of malate, which comprises
(a) increasing or generating the activity of a ATP-binding component of a transport system in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of malate, which comprises
(a) increasing or generating the activity of a membrane transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a membrane transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citrate, which comprises
(a) increasing or generating the activity of a glycerate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrate or a composition comprising citrate in said non-human organism or in the culture medium surrounding said non-human organism.

Accordingly, the present invention relates to a process for the production of fumarate, which comprises
(a) increasing or generating the activity of a isochorismate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a isochorismate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of fumarate, which comprises
(a) increasing or generating the activity of a zinc transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating the activity of a b0801-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of malate, which comprises
(a) increasing or generating the activity of a leucyl/phenylalanyl-tRNA-protein transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of vitamin c and/or threonic acid, which comprises
(a) increasing or generating the activity of a methylglyoxal synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of malate, which comprises
(a) increasing or generating the activity of a b1003-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of malate, which comprises
(a) increasing or generating the activity of a membrane protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a b1285-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of malate, which comprises
(a) increasing or generating the activity of a phenylacetic acid degradation protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of malate, which comprises
(a) increasing or generating the activity of a b1425-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a lipoprotein precursor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citramalate, which comprises
(a) increasing or generating the activity of a b1445-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of citramalate, which comprises
(a) increasing or generating the activity of a b1522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of malate, which comprises
(a) increasing or generating the activity of a b1522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of fumarate, which comprises
(a) increasing or generating the activity of a Fe—S subunit of oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of fumarate, which comprises
(a) increasing or generating the activity of a fumarate hydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating the activity of a electron transport complex protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of succinate, which comprises
(a) increasing or generating the activity of a methylglyoxalase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citramalate, which comprises
(a) increasing or generating the activity of a b1670-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of malate, which comprises
(a) increasing or generating the activity of a b1670-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of citramalate, which comprises
(a) increasing or generating the activity of a b1672-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of malate, which comprises
(a) increasing or generating the activity of a b1672-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of vitamin c and/or threonic acid, which comprises
(a) increasing or generating the activity of a b1672-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of malate, which comprises
(a) increasing or generating the activity of a b1742-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a b1742-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of succinate, which comprises
(a) increasing or generating the activity of a arginine N-succinyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of vitamin c and/or threonic acid, which comprises
(a) increasing or generating the activity of a ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of malate, which comprises
(a) increasing or generating the activity of a protease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of vitamin c and/or threonic acid, which comprises
(a) increasing or generating the activity of a dATP pyrophosphohydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of succinate, which comprises
(a) increasing or generating the activity of a b1898-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating the activity of a flagellar M-ring protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a flagellar M-ring protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a flagellar M-ring protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of malate, which comprises
(a) increasing or generating the activity of a O-antigen chain length determinant in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of fumarate, which comprises
(a) increasing or generating the activity of a b2399-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating the activity of a b2399-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of vitamin c and/or threonic acid, which comprises
(a) increasing or generating the activity of a b2399-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of vitamin c and/or threonic acid, which comprises
(a) increasing or generating the activity of a cysteine synthase A in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of vitamin c and/or threonic acid, which comprises
(a) increasing or generating the activity of a cysteine synthase B in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of fumarate, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a murein transglycosylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of succinate, which comprises
(a) increasing or generating the activity of a 2-C-methyl-D-erythritol 4-phosphate cytidylyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of succinate, which comprises
(a) increasing or generating the activity of a amino-acid acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of succinate, which comprises
(a) increasing or generating the activity of a xanthine dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of succinate, which comprises
(a) increasing or generating the activity of a cystathionine beta-lyase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating the activity of a methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of fumarate, which comprises
(a) increasing or generating the activity of a lipopolysaccharide core biosynthesis protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating the activity of a b3777-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of malate, which comprises
(a) increasing or generating the activity of a b3791-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a b3791-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citramalate, which comprises
(a) increasing or generating the activity of a b3814-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating the activity of a b3817-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of citramalate, which comprises
(a) increasing or generating the activity of a threonine efflux protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of malate, which comprises
(a) increasing or generating the activity of a threonine efflux protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a b3989-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of fumarate, which comprises
(a) increasing or generating the activity of a b4029-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a b4029-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a b4029-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating the activity of a b4050-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a b4050-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of citramalate, which comprises
(a) increasing or generating the activity of a b4056-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of citrate, which comprises
(a) increasing or generating the activity of a aspartase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrate or a composition comprising citrate in said non-human organism or in the culture medium surrounding said non-human organism.

Accordingly, the present invention relates to a process for the production of fumarate, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of vitamin c and/or threonic acid, which comprises
(a) increasing or generating the activity of a purine-nucleoside phosphorylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of succinate, which comprises
(a) increasing or generating the activity of a lysine decarboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of succinate, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating the activity of a GM02LC17485-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of citramalate, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of citramalate, which comprises
(a) increasing or generating the activity of a GM02LC5744-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating the activity of a GM02LC5744-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of citramalate, which comprises
(a) increasing or generating the activity of a heat shock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating the activity of a agmatinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a ABC transporter ATP binding component in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a ABC transporter ATP binding component in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating the activity of a oxireductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating the activity of a phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citramalate, which comprises
(a) increasing or generating the activity of a CDP-diacylglycerol-glycerol-3-phosphate 3-phosphatidyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of malate, which comprises
(a) increasing or generating the activity of a CDP-diacylglycerol-glycerol-3-phosphate 3-phosphatidyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a adenylate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citramalate, which comprises
(a) increasing or generating the activity of a cation-transporting ATPase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of malate, which comprises
(a) increasing or generating the activity of a cation-transporting ATPase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of fumarate, which comprises
(a) increasing or generating the activity of a fumarate hydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of citramalate, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating the activity of a bifunctional purine biosynthesis protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of citramalate, which comprises
(a) increasing or generating the activity of a malate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of succinate, which comprises
(a) increasing or generating the activity of a malate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of glycerate, which comprises
(a) increasing or generating the activity of a dihydrolipoamide dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

Accordingly, the present invention relates to a process for the production of succinate, which comprises
(a) increasing or generating the activity of a dihydrolipoamide dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of succinate, which comprises
(a) increasing or generating the activity of a slr1170-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating the activity of a phosphate import ATP-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating the activity of a gamma-glutamyltranspeptidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of malate, which comprises
(a) increasing or generating the activity of a photosystem II protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of fumarate, which comprises
(a) increasing or generating the activity of a malonyl CoA-acyl carrier protein transacylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating the activity of a malonyl CoA-acyl carrier protein transacylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of glycerate, which comprises
(a) increasing or generating the activity of a photosystem I reaction centre subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

Accordingly, the present invention relates to a process for the production of fumarate, which comprises
(a) increasing or generating the activity of a Sec-independent protein translocase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating the activity of a Sec-independent protein translocase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citramalate, which comprises
(a) increasing or generating the activity of a 2-isopropylmalate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating the activity of a multiple antibiotic resistance protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating the activity of a multiple antibiotic resistance protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating the activity of a TTC1386-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a TTC1386-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a TTC1386-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citramalate, which comprises
(a) increasing or generating the activity of a oxidoreductase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of glycerate, which comprises
(a) increasing or generating the activity of a oxidoreductase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

Accordingly, the present invention relates to a process for the production of malate, which comprises
(a) increasing or generating the activity of a oxidoreductase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of fumarate, which comprises
(a) increasing or generating the activity of a XM_{—}473199-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a XM_{—}473199-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a XM_{—}473199-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of succinate, which comprises
(a) increasing or generating the activity of a glutamate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citramalate, which comprises
(a) increasing or generating the activity of a acetyl-CoA hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of pyruvate, which comprises
(a) increasing or generating the activity of a acetyl-CoA hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of pyruvate or a composition comprising pyruvate in said non-human organism or in the culture medium surrounding said non-human organism.

Accordingly, the present invention relates to a process for the production of malate, which comprises
(a) increasing or generating the activity of a pyruvate carboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of fumarate, which comprises
(a) increasing or generating the activity of a ycr102c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of fumarate, which comprises
(a) increasing or generating the activity of a YDL159W-A-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of fumarate, which comprises
(a) increasing or generating the activity of a coproporphyrinogen III oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating the activity of a coproporphyrinogen III oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of malate, which comprises
(a) increasing or generating the activity of a ygl237c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a ygl237c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating the activity of a YGR193C-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a YGR193C-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a YGR193C-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of malate, which comprises
(a) increasing or generating the activity of a ygr221 c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of citrate, which comprises
(a) increasing or generating the activity of a glycerol kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citrate or a composition comprising citrate in said non-human organism or in the culture medium surrounding said non-human organism.

Accordingly, the present invention relates to a process for the production of malate, which comprises
(a) increasing or generating the activity of a yhr131c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the pro-duction of malate or a composition comprising malate in said non-human organism or in the culture medium surrounding said non-human organism.

Accordingly, the present invention relates to a process for the production of succinate, which comprises
(a) increasing or generating the activity of a yhr207c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of glycerate, which comprises
(a) increasing or generating the activity of a DnaJ-like chaperone in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

Accordingly, the present invention relates to a process for the production of oxalate, which comprises
(a) increasing or generating the activity of a hexose transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of oxalate or a composition comprising oxalate in said non-human organism or in the culture medium surrounding said non-human organism.

Accordingly, the present invention relates to a process for the production of citramalate, which comprises
(a) increasing or generating the activity of a branched chain aminotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of fumarate, which comprises
(a) increasing or generating the activity of a polygalacturonase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a polygalacturonase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of malate, which comprises
(a) increasing or generating the activity of a YKL038W-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of vitamin c and/or threonic acid, which comprises
(a) increasing or generating the activity of a YKL140W-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of fumarate, which comprises
(a) increasing or generating the activity of a ylr065c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating the activity of a isocitrate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating the activity of a ylr326w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of glycerate, which comprises
(a) increasing or generating the activity of a ylr326w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerate or a composition comprising glycerate in said non-human organism or in the culture medium surrounding said non-human organism.

Accordingly, the present invention relates to a process for the production of vitamin c and/or threonic acid, which comprises
(a) increasing or generating the activity of a ylr326w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of fumarate, which comprises
(a) increasing or generating the activity of a yml084w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of succinate, which comprises
(a) increasing or generating the activity of a yml084w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citramalate, which comprises
(a) increasing or generating the activity of a yol073c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of malate, which comprises
(a) increasing or generating the activity of a yol073c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of vitamin c and/or threonic acid, which comprises
(a) increasing or generating the activity of a yol160w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of malate, which comprises
(a) increasing or generating the activity of a acetyl-CoA acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of vitamin c and/or threonic acid, which comprises
(a) increasing or generating the activity of a ammonium transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of fumarate, which comprises
(a) increasing or generating the activity of a 47266012-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a 47266012-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a 47266012-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of fumarate, which comprises
(a) increasing or generating the activity of a 49747384_SOYBEAN-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fumarate or a composition comprising fumarate in said non-human organism or in the culture medium surrounding said non-human organism.

Accordingly, the present invention relates to a process for the production of malate, which comprises
(a) increasing or generating the activity of a 49747384_SOYBEAN-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of malate or a composition comprising malate in said non-human organism or in the culture medium surrounding said non-human organism.

Accordingly, the present invention relates to a process for the production of succinate, which comprises
(a) increasing or generating the activity of a 49747384_SOYBEAN-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of vitamin c and/or threonic acid, which comprises
(a) increasing or generating the activity of a 49747384_SOYBEAN-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of vitamin c and/or threonic acid or a composition comprising vitamin c and/or threonic acid in said non-human organism or in the culture medium surrounding said non-human organism.

Accordingly, the present invention relates to a process for the production of citramalate, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of succinate, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of succinate or a composition comprising succinate in said non-human organism or in the culture medium surrounding said non-human organism.

Accordingly, the present invention relates to a process for the production of citramalate, which comprises
(a) increasing or generating the activity of a glucose-6-phosphate 1-dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate or a composition comprising citramalate in said non-human organism or in the culture medium surrounding said non-human organism.

Accordingly, the present invention relates to a process for the production of citramalate, which comprises
(a) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical citramalate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical citramalate;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of citramalate, or a composition comprising citramalate in said non-human organism or in the culture medium surrounding said non-human organism.

Accordingly, the present invention relates to a process for the production of citrate, which comprises
(a) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical citrate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical citrate;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of citrate, or a composition comprising citrate in said non-human organism or in the culture medium surrounding said non-human organism.

Accordingly, the present invention relates to a process for the production of fumarate, which comprises
(a) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical fumarate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical fumarate;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of fumarate, or a composition comprising fumarate in said non-human organism or in the culture medium surrounding said non-human organism.

Accordingly, the present invention relates to a process for the production of glycerate, which comprises
(a) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical glycerate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical glycerate;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of glycerate, or a composition comprising glycerate in said non-human organism or in the culture medium surrounding said non-human organism.

Accordingly, the present invention relates to a process for the production of malate, which comprises
(a) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical malate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical malate;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of malate, or a composition comprising malate in said non-human organism or in the culture medium surrounding said non-human organism.

Accordingly, the present invention relates to a process for the production of oxalate, which comprises
(a) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical oxalate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical oxalate;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of oxalate, or a composition comprising oxalate in said non-human organism or in the culture medium surrounding said non-human organism.

Accordingly, the present invention relates to a process for the production of pyruvate, which comprises
(a) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical pyruvate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical pyruvate;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of pyruvate, or a composition comprising pyruvate in said non-human organism or in the culture medium surrounding said non-human organism.

Accordingly, the present invention relates to a process for the production of succinate, which comprises
(a) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical succinate; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical succinate;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of succinate, or a composition comprising succinate in said non-human organism or in the culture medium surrounding said non-human organism.

Accordingly, the present invention relates to a process for the production of vitamin c and/or threonic acid, which comprises
(a) increasing or generating the activity of a polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, whereby the respective line disclose in column 7 the fine chemical vitamin c and/or threonic acid; or
increasing or generating the activity of an expression product of one or more nucleic acid molecule(s) comprising a polynucleotide as depicted in the respective line in column 5 or 8 of Table I, application no. 15, preferably the coding region thereof, or a homolog or a fragment thereof, whereby the respective line disclose in column 7 the fine chemical vitamin c and/or threonic acid;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of vitamin c and/or threonic acid, or a composition comprising vitamin c and/or threonic acid in said non-human organism or in the culture medium surrounding said non-human organism.

In a further embodiment the activity of the polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, is increased or generated non-targeted in the above-mentioned process in a microorganism or plant or a part thereof.

In a further embodiment said polypeptide has the activity of the respective polypeptide represented by a protein comprising a polypeptide as depicted in the respective line in column 5 of Table II, application no. 15.

In a further embodiment the activity of the expression product of one or more nucleic acid molecule(s) comprising a polynucleotide as depicted in the respective line in column 5 or 8 of Table I, application no. 15, preferably the coding region thereof, or a homolog or a fragment thereof, is increased or generated non-targeted in the above-mentioned process in a microorganism or plant or a part thereof.

In a further embodiment the activity of the polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, is increased or generated in the above-mentioned process in an organelle preferably in plastids or mitochondria, especially in plastids, of a microorganism or plant.

In a further embodiment said polypeptide has the activity of the respective polypeptide represented by a protein comprising a polypeptide as depicted in the respective line in column 5 of Table II, application no. 15.

In a further embodiment the activity of the expression product of one or more nucleic acid molecule(s) comprising a polynucleotide as depicted in the respective line in column 5 or 8 of Table I, application no. 15, preferably the coding region thereof, or a homolog or a fragment thereof, is increased or generated in the above-mentioned process in an organelle, preferably in plasids or mitochondria, especially in plastids, of a microorganism or plant.

In a further embodiment the activity of the polypeptide comprising a polypeptide as depicted in the respective line in column 5 or 8 of Table II, application no. 15, or a homolog or a fragment thereof, a consensus sequence or at least one polypeptide motif as depicted in the respective line in column 8 of Table IV, application no. 15, is increased or generated in the above-mentioned process in the cyctosol of a cell, of a microorganism or plant.

In a further embodiment said polypeptide has the activity of the respective polypeptide represented by a protein comprising a polypeptide as depicted in the respective line in column 5 of Table II, application no. 15.

In a further embodiment the activity of the expression product of one or more nucleic acid molecule(s) comprising a polynucleotide as depicted in the respective line in column 5 or 8 of Table I, application no. 15, preferably the coding region thereof, or a homolog or a fragment thereof, is increased or generated in the above-mentioned process in the cytosol of a cell, of a microorganism or plant.

for the disclosure of this paragraph see [0039.1.7.7] above

for the disclosure of this paragraph see [0040.1.7.7] above

for the disclosure of this paragraph see [0041.1.7.7] above

for the disclosure of this paragraph see [0042.1.7.7] above

for the disclosure of this paragraph see [0043.1.7.7] above

for the disclosure of this paragraph see [0044.1.7.7] above

for the disclosure of this paragraph see [0045.1.7.7] above

for the disclosure of this paragraph see [0046.1.7.7] above

for the disclosure of this paragraph see [0047.1.7.7] above

for the disclosure of this paragraph see [0048.1.7.7] above

for the disclosure of this paragraph see [0049.1.7.7] above

for the disclosure of this paragraph see [0050.1.7.7] above

for the disclosure of this paragraph see [0051.1.7.7] above

for the disclosure of this paragraph see [0052.1.7.7] above

for the disclosure of this paragraph see [0053.1.7.7] above

for the disclosure of this paragraph see [0054.1.7.7] above

for the disclosure of this paragraph see [0055.1.7.7] above

for the disclosure of this paragraph see [0056.1.7.7] above

for the disclosure of this paragraph see [0057.1.7.7] above

for the disclosure of this paragraph see [0058.1.7.7] above

for the disclosure of this paragraph see [0059.1.7.7] above

for the disclosure of this paragraph see [0060.1.7.7] above

for the disclosure of this paragraph see [0061.1.7.7] above

for the disclosure of this paragraph see [0062.1.7.7] above

for the disclosure of this paragraph see [0063.1.7.7] above

for the disclosure of this paragraph see [0064.1.7.7] above

for the disclosure of this paragraph see [0065.1.7.7] above

for the disclosure of this paragraph see [0066.1.7.7] above

As mentioned above the nucleic acid sequences coding for the proteins as shown in Table II, application no. 15, column 5 or 8, or homologs or fragments thereof, are joined to a nucleic acid sequence encoding a plastidic transit peptide, e.g, if for the nucleic acid molecule in Table I, column 6 the term “plastidic” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide coding sequence is fused in frame to the nucleic acid sequence coding for proteins as shown in Table II, application no. 15, column 5 or 8, or homologs or fragments thereof, e.g., if for the nucleic acid molecule in Table I, column 6 the term “plastidic” is indicated.

for the disclosure of this paragraph see [0068.1.7.7] above

for the disclosure of this paragraph see [0069.1.7.7] above

for the disclosure of this paragraph see [0070.1.7.7] above

for the disclosure of this paragraph see [0071.1.7.7] above

for the disclosure of this paragraph see [0072.1.7.7] above

As mentioned above the nucleic acid sequences coding for the proteins as shown in the respective line in Table II, application no. 15, column 5 or 8, or homologs or fragments thereof, are joined to a nucleic acid sequence encoding a mitochondric transit peptide, e.g., if for the respective nucleic acid molecule in Table I, column 6 the term “mitochondric” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in the respective line in Table II, application no. 15, column 5 or 8, or homologs or fragments thereof, e.g., if for the nucleic acid molecule in the respective line in Table I, column 6 the term “mitochondric” is indicated.

for the disclosure of this paragraph see [0074.1.7.7] above

for the disclosure of this paragraph see [0075.1.7.7] above

Nucleic acid sequences coding for the transit peptides may be chemically synthesized either in part or wholly according to structure of transit peptide sequences disclosed in the prior art. Said natural or chemically synthesized sequences can be directly linked to the sequences encoding the mature protein or via a linker nucleic acid sequence, which may be typically less than 500 base pairs, preferably less than 450, 400, 350, 300, 250 or 200 base pairs, more preferably less than 150, 100, 90, 80, 70, 60, 50, 40 or 30 base pairs and most preferably less than 25, 20, 15, 12, 9, 6 or 3 base pairs in length and are in frame to the coding sequence. Furthermore favorable nucleic acid sequences encoding transit peptides may comprise sequences derived from more than one biological and/or chemical source and may include a nucleic acid sequence derived from the amino-terminal region of the mature protein, which in its native state is linked to the transit peptide. In a preferred embodiment of the invention said amino-terminal region of the mature protein is typically less than 150 amino acids, preferably less than 140, 130, 120, 110, 100 or 90 amino acids, more preferably less than 80, 70, 60, 50, 40, 35, 30, 25 or 20 amino acids and most preferably less than 19, 18, 17, 16, 15, 14, 13, 12, 11 or 10 amino acids in length. But even shorter or longer stretches are also possible. In addition target sequences, which facilitate the transport of proteins to other cell compartments such as the vacuole, endoplasmic reticulum, golgi complex, glyoxysomes, peroxisomes, mitochondria (in case of a plastidal transit peptide) or plastids, especially chloroplasts (in case of a mitochondric transit peptide) may be also part of the inventive nucleic acid sequence. The proteins translated from said inventive nucleic acid sequences are a kind of fusion proteins that means the nucleic acid sequences encoding the plastidal transit peptide for example the ones shown in Table a, preferably the last one of the Table a are joint to the respective nucleic acid sequences shown in Table I, application no. 15, columns 5 or 8, especially the coding region thereof, or homologs or fragments thereof, in case of nucleic acid sequences being expressed in plastids, and that means the nucleic acid sequences encoding the mitochondric transit peptide for example the ones shown in Table b, preferably the last one of the Table b are joint to the respective nucleic acid sequences shown in Table I, application no. 15, columns 5 or 8, especially the coding region thereof, or homologs or fragments thereof, in case of nucleic acid sequences being expressed in mitochondria. The person skilled in the art is able to join said sequences in a functional manner. Advantageously the transit peptide part is cleaved off from the protein part shown in Table II, application no. 15, columns 5 or 8, or a homolog or fragment thereof, during or after the transport into the organelles, preferably into the plastids or mitochondria, respectively.

All products of the cleavage of the preferred transit peptide shown in the last line of Table a have preferably the N-terminal amino acid sequences QIA CSS or QIA EFQLTT in front of the start methionine of the respective protein mentioned in Table II, application no. 15, column 5 or 8. or homologs or fragments thereof. Other short amino acid sequences of an range of 1 to 20 amino acids preferable 2 to 15 amino acids, more preferable 3 to 10 amino acids most preferably 4 to 8 amino acids are also possible in front of the start methionine of the protein mentioned in Table II, application no. 15, columns 5 or 8, or homologs or fragments thereof. In case of the amino acid sequence QIA CSS the three amino acids in front of the start methionine are stemming from the LIC (=ligatation independent cloning) cassette. Said short amino acid sequence is preferred in the case of the expression of E. coli genes. In case of the amino acid sequence QIA EFQLTT the six amino acids in front of the start methionine are stemming from the LIC cassette. Said short amino acid sequence is preferred in the case of the expression of S. cerevisiae genes. The skilled worker knowns that other short sequences are also useful in the expression of the respective nucleic acid molecule comprising a polynucleotide as depicted in Table I, application no. 15, column 5 or 8, preferably the coding region thereof, or a homolog or a fragment thereof. Furthermore the skilled worker is aware of the fact that there is not a need for such short sequences in the expression of the genes.

for the disclosure of this paragraph see [0077.1.7.7] above

for the disclosure of this paragraph see [0078.1.7.7] above

Alternatively to the targeting of the respective sequences shown in Table II, application no. 15, columns 5 or 8, or homologs or fragments thereof, preferably of sequences in general encoded in the nucleus with the aid of the targeting sequences mentioned for example in Table a alone, Table b alone, or in combination with other targeting sequences preferably into plastids or mitochondria, respectively, the nucleic acids of the invention can directly be introduced into the plastidal or mitochondrial genome. Therefore in a preferred embodiment the respective nucleic acid sequences shown in the respective line in Table I, application no. 15, columns 5 or 8, preferably the coding region thereof, or homologs or fragments thereof, are directly introduced and expressed in plastids e.g., if for the nucleic acid molecule in the respective line in Table I, column 6, the term “plastidic” is indicated, or are directly introduced and expressed in mitochondria e.g., if for the nucleic acid molecule in the respective line in Table I, column 6 the term “mitochondric” is indicated.

The term “introduced” in the context of this specification shall mean the insertion of a nucleic acid sequence into the organism by means of a “transfection”, “transduction” or preferably by “transformation”.

A plastid, such as a chloroplast, or a mitochondrion, has been “transformed” by an exogenous (preferably foreign) nucleic acid sequence if a nucleic acid sequence has been introduced into the plastid or mitochondrion that means that this sequence has crossed the membrane or the membranes of the plastid or of the mitochondrion. The exogenous, preferably foreign, DNA may be integrated (covalently linked) into plastid or mitochondrial DNA making up the genome of the plastid or mitochondrion, or it may remain unintegrated (e.g., by including a chloroplast origin of replication). “Stably” integrated DNA sequences are those, which are inherited through plastid or mitochondrion replication, thereby transferring new plastids or mitochondria, with the features of the integrated DNA sequence to the progeny.

[0080.1.7.15] to [0083.1.7.15] for the disclosure of these paragrapsh see [0080.1.7.7] to [0083.1.7.7] above

For the process of the present invention it may be of great advantage that by transforming the plastids the intraspecies specific transgene flow is blocked, because a lot of species such as corn, cotton and rice have a strict maternal inheritance of plastids. By placing the respective genes specified in the respective line in Table I, application no. 15, columns 5 or 8, preferably the coding region, or homologs or fragments thereof, e.g. if for the nucleic acid molecule in the respective line in column 6 of Table I the term “plastidic” is indicated, in the plastids of plants, these genes will not be present in the pollen of said plants.

A further preferred embodiment of the invention relates to the use of so called “chloroplast localization sequences”, in which a first RNA sequence or molecule is capable of transporting or “chaperoning” a second RNA sequence, such as a RNA sequence transcribed from the sequences depicted in the respective line in Table I, application no. 15, columns 5 or 8, preferably the coding region thereof, or a homolog or a fragment thereof, or a sequence encoding a protein, as depicted in the respective line in Table II, application no. 15, columns 5 and 8, or a homolog or a fragment thereof, from an external environment inside a cell or outside a plastid into a chloroplast. In one embodiment the chloroplast localization signal is substantially similar or complementary to a complete or intact viroid sequence. The chloroplast localization signal may be encoded by a DNA sequence, which is transcribed into the chloroplast localization RNA. The term “viroid” refers to a naturally occurring single stranded RNA molecule (Flores, C. R. Acad. Sci. III. 324 (10), 943 (2001)). Viroids usually contain about 200-500 nucleotides and generally exist as circular molecules. Examples of viroids that contain chloroplast localization signals include but are not limited to ASBVd, PLMVd, CChMVd and ELVd. The viroid sequence or a functional part of it can be fused to the sequences depicted in the respective line in Table I, application no. 15, columns 5 or 8, preferably the coding region thereof, or a homolog or a fragment thereof, or a sequence encoding a protein, as depicted in the respective line in Table II, application no. 15, columns 5 or 8, or a homolog or a fragment thereof, in such a manner that the viroid sequence transports a sequence transcribed from a sequence as depicted in the respective line in Table I, application no. 15 columns 5 or 8, preferably the coding region thereof, or a homolog or a fragment thereof, or a sequence encoding a protein as depicted in the respective line in Table II, application no. 15 columns 5 or 8, or a homolog or a fragment thereof, into the chloroplasts, e.g. if for the nucleic acid molecule in column 6 of Table I the term “plastidic” is indicated. A preferred embodiment uses a modified ASBVd (Navarro et al., Virology. 268 (1), 218 (2000)).

In a further specific embodiment the protein to be expressed in the plastids such as the proteins depicted in the respective line in Table II, application no. 15, columns 5 or 8, or a homolog or a fragment thereof, e.g. if for the nucleic acid molecule in the respective line in column 6 of Table I the term “plastidic” is indicated, are encoded by different nucleic acids. Such a method is disclosed in WO 2004/040973, which shall be incorporated by reference. WO 2004/040973 teaches a method, which relates to the translocation of an RNA corresponding to a gene or gene fragment into the chloroplast by means of a chloroplast localization sequence. The genes, which should be expressed in the plant or plant cells, are split into nucleic acid fragments, which are introduced into different compartments in the plant e.g. the nucleus, the plastids and/or mitochondria. Additionally plant cells are described in which the chloroplast contains a ribozyme fused at one end to an RNA encoding a fragment of a protein used in the inventive process such that the ribozyme can trans-splice the translocated fusion RNA to the RNA encoding the gene fragment to form and as the case may be reunite the nucleic acid fragments to an intact mRNA encoding a functional protein for example as disclosed in the respective line in Table II, application no. 15, columns 5 or 8, or a homolog or a fragment thereof.

In a preferred embodiment of the invention the nucleic acid sequences as shown in the respective line in Table I, application no. 15, columns 5 or 8, preferably the coding region thereof, or homologs or fragments thereof, e.g. if for the nucleic acid molecule in the respective line in column 6 of Table I the term “plastidic” is indicated, used in the inventive process are transformed into plastids, which are metabolically active. Those plastids should preferably maintain at a high copy number in the plant or plant tissue of interest, most preferably the chloroplasts found in green plant tissues, such as leaves or cotyledons or in seeds.

For a good expression in the plastids the nucleic acid sequences as shown in the respective line in Table I, application no. 15, columns 5 or 8, preferably the coding region thereof, or homologs or fragments thereof, e.g. if for the nucleic acid molecule in the respective line in column 6 of Table I the term “plastidic” is indicated, are introduced into an expression cassette using preferably a promoter and terminater, which are active in plastids preferably a chloroplast promoter. Examples of such promoters include the psbA promoter from the gene from spinach or pea, the rbcL promoter, and the atpB promoter from corn.

[0089.1.7.15] to [0092.1.7.15] for the disclosure of these paragraphs see [0089.1.7.7] to [0092.1.7.7] above

Advantageously the process for the production of the fine chemical citramalate, citrate, fumarate, glycerate, malate, oxalate, pyruvate, succinate, and/or vitamin c and/or threonic acid leads to an enhanced production of the respective fine chemical. The terms “enhanced” or “increase” mean at least a 10%, 20%, 30%, 40% or 50%, preferably at least 60%, 70%, 80%, 90% or 100%, more preferably 150%, 200%, 300%, 400% or 500% higher production of the respective fine chemical citramalate, citrate, fumarate, glycerate, malate, oxalate, pyruvate, succinate, and/or vitamin c and/or threonic acid in comparison to the wild-type as defined above, e.g. that means in comparison to a non-human organism without the aforementioned modification of the activity of a protein as shown in the respective line in Table II, application no. 15, column 5 or 8, or a fragment or a homolog thereof. The modification of the activity of a protein as shown in the respective line in Table II, application no. 15, column 5 or 8, or a homolog or a fragment thereof, or their combination can be achieved by joining the protein to a respective transit peptide, e.g. if for the respective encoding nucleic acid molecule in column 6 of Table I the term “plastidic” or “mitochondric” is indicated.

Surprisingly it was found, that the transgenic expression of a protein as shown in the respective line in Table II, application no. 15, column 5, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, conferred a production of or an increase in citramalate, citrate, fumarate, glycerate, malate, oxalate, pyruvate, succinate, and/or vitamin c and/or threonic acid, respectively, to the transgenic non-human organism as compared to a corresponding non-transformed wild type.

In one embodiment the transgenic expression of a protein as shown in the respective line in Table II, application no. 15, column 5 or 8, or a homolog or a fragment thereof, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, confers a production of or an increase in citramalate, citrate, fumarate, glycerate, malate, oxalate, pyruvate, succinate, and/or vitamin c and/or threonic acid, respectively, to the transgenic non-human organism as compared to a corganism responding non-transformed wild type.

Accordingly, in one embodiment, an increase of succinate in a non-human organism, as compared to a corresponding non-transformed wild type non-human organism, is conferred in the process of the invention, if the activity of a polypeptide showing the activity of a protein phosphatase, or if the activity of the polypeptide At1g07430, preferably represented by SEQ ID NO. 17452, or a homolog or fragment thereof, or if the activity of a polypeptide encoded by a nucleic acid molecule comprising the nucleic acid SEQ ID NO. 17451, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 17451 or polypeptide SEQ ID NO. 17452, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 47 to 67-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a At1g13930-protein, or if the activity of the polypeptide At1g13930, preferably represented by SEQ ID NO. 133002, or a homolog or fragment thereof, or if the activity of a polypeptide encoded by a nucleic acid molecule comprising the nucleic acid SEQ ID NO. 133001, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 133001 or polypeptide SEQ ID NO. 133002, respectively, or a homolog or a fragment thereof, is increased or generated, or if the activity At1g13930-protein is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 82 to 90-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a At1g13930-protein, or if the activity of the polypeptide At1g13930, preferably represented by SEQ ID NO. 133002, or a homolog or fragment thereof, or if the activity of a polypeptide encoded by a nucleic acid molecule comprising the nucleic acid SEQ ID NO. 133001, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 133001 or polypeptide SEQ ID NO. 133002, respectively, or a homolog or a fragment thereof, is increased or generated, or if the activity At1g13930-protein is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 33 to 79-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a eukaryotic translation initiation factor 5, or if the activity of the polypeptide At1g36730, preferably represented by SEQ ID NO. 17969, or a homolog or fragment thereof, or if the activity of a polypeptide encoded by a nucleic acid molecule comprising the nucleic acid SEQ ID NO. 17968, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 17968 or polypeptide SEQ ID NO. 17969, respectively, or a homolog or a fragment thereof, is increased or generated, or if the activity eukaryotic translation initiation factor 5 is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 65 to 137-percent is conferred as compared to a corresponding non-transformed wild type non-human organism.

Accordingly, in one embodiment, an increase of vitamin c and/or threonic acid in a non-human organism, as compared to a corresponding non-transformed wild type non-human organism, is conferred in the process of the invention, if the activity of a polypeptide showing the activity of a eukaryotic translation initiation factor 5, or if the activity of the polypeptide At1g36730, preferably represented by SEQ ID NO. 17969, or a homolog or fragment thereof, or if the activity of a polypeptide encoded by a nucleic acid molecule comprising the nucleic acid SEQ ID NO. 17968, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 17968 or polypeptide SEQ ID NO. 17969, respectively, or a homolog or a fragment thereof, is increased or generated, or if the activity eukaryotic translation initiation factor 5 is increased or generated in a non-human organism, like a microorganism or a plant cell, plant or part thereof, especially with non-targeted localization, whereby the respective line disclose in column 7 the fine chemical vitamin c and/or threonic acid. For example, an increase of the vitamin c and/or threonic acid of at least 1 percent, particularly in a range of 31 to 56-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a transcriptional regulator, or if the activity of the polypeptide At1g43850, preferably represented by SEQ ID NO. 18071, or a homolog or fragment thereof, or if the activity of a polypeptide encoded by a nucleic acid molecule comprising the nucleic acid SEQ ID NO. 18070, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 18070 or polypeptide SEQ ID NO. 18071, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 63 to 108-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a protein phosphatase, or if the activity of the polypeptide At1g48040, preferably represented by SEQ ID NO. 813, or a homolog or fragment thereof, or if the activity of a polypeptide encoded by a nucleic acid molecule comprising the nucleic acid SEQ ID NO. 812, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 812 or polypeptide SEQ ID NO. 813, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 49 to 81-percent is conferred as compared to a corresponding non-transformed wild type non-human organism.

Accordingly, in one embodiment, an increase of vitamin c and/or threonic acid in a non-human organism, as compared to a corresponding non-transformed wild type non-human organism, is conferred in the process of the invention, if the activity of a polypeptide showing the activity of a protein phosphatase, or if the activity of the polypeptide At1g72770, preferably represented by SEQ ID NO. 19420, or a homolog or fragment thereof, or if the activity of a polypeptide encoded by a nucleic acid molecule comprising the nucleic acid SEQ ID NO. 19419, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 19419 or polypeptide SEQ ID NO. 19420, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical vitamin c and/or threonic acid. For example, an increase of the vitamin c and/or threonic acid of at least 1 percent, particularly in a range of 31 to 109-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a CBL-interacting protein kinase, or if the activity of the polypeptide At2g30360, preferably represented by SEQ ID NO. 19920, or a homolog or fragment thereof, or if the activity of a polypeptide encoded by a nucleic acid molecule comprising the nucleic acid SEQ ID NO. 19919, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 19919 or polypeptide SEQ ID NO. 19920, respectively, or a homolog or a fragment thereof, is increased or generated, or if the activity CBL-interacting protein kinase is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 75 to 420-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a CBL-interacting protein kinase, or if the activity of the polypeptide At2g30360, preferably represented by SEQ ID NO. 19920, or a homolog or fragment thereof, or if the activity of a polypeptide encoded by a nucleic acid molecule comprising the nucleic acid SEQ ID NO. 19919, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 19919 or polypeptide SEQ ID NO. 19920, respectively, or a homolog or a fragment thereof, is increased or generated, or if the activity CBL-interacting protein kinase is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 52 to 364-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a CBL-interacting protein kinase, or if the activity of the polypeptide At2g30360, preferably represented by SEQ ID NO. 19920, or a homolog or fragment thereof, or if the activity of a polypeptide encoded by a nucleic acid molecule comprising the nucleic acid SEQ ID NO. 19919, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 19919 or polypeptide SEQ ID NO. 19920, respectively, or a homolog or a fragment thereof, is increased or generated, or if the activity CBL-interacting protein kinase is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 24 to 75-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a phenylalanine ammonia-lyase, or if the activity of the polypeptide At2g37040, preferably represented by SEQ ID NO. 133043, or a homolog or fragment thereof, or if the activity of a polypeptide encoded by a nucleic acid molecule comprising the nucleic acid SEQ ID NO. 133042, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 133042 or polypeptide SEQ ID NO. 133043, respectively, or a homolog or a fragment thereof, is increased or generated, or if the activity phenylalanine ammonia-lyase is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 70 to 89-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a thioredoxin reductase, or if the activity of the polypeptide At2g41680, preferably represented by SEQ ID NO. 133350, or a homolog or fragment thereof, or if the activity of a polypeptide encoded by a nucleic acid molecule comprising the nucleic acid SEQ ID NO. 133349, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 133349 or polypeptide SEQ ID NO. 133350, respectively, or a homolog or a fragment thereof, is increased or generated, or if the activity thioredoxin red uctase is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 34 to 91-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a citrate synthase, or if the activity of the polypeptide At2g44350, preferably represented by SEQ ID NO. 120811, or a homolog or fragment thereof, or if the activity of a polypeptide encoded by a nucleic acid molecule comprising the nucleic acid SEQ ID NO. 120810, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 120810 or polypeptide SEQ ID NO. 120811, respectively, or a homolog or a fragment thereof, is increased or generated, or if the activity citrate synthase is increased or generated in a non-human organism, like a microorganism or a plant cell, plant or part thereof, especially with plastidic localization, whereby the respective line disclose in column 7 the fine chemical succinate. For example, an increase of the succinate of at least 1 percent, particularly in a range of 46 to 132-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a pyruvate kinase, or if the activity of the polypeptide At3g04050, preferably represented by SEQ ID NO. 21498, or a homolog or fragment thereof, or if the activity of a polypeptide encoded by a nucleic acid molecule comprising the nucleic acid SEQ ID NO. 21497, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 21497 or polypeptide SEQ ID NO. 21498, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line disclose in column 7 the fine chemical citramalate. For example, an increase of the citramalate of at least 1 percent, particularly in a range of 49 to 721-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a pyruvate kinase, or if the activity of the polypeptide At3g04050, preferably represented by SEQ ID NO. 21498, or a homolog or fragment thereof, or if the activity of a polypeptide encoded by a nucleic acid molecule comprising the nucleic acid SEQ ID NO. 21497, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 21497 or polypeptide SEQ ID NO. 21498, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line disclose in column 7 the fine chemical succinate. For example, an increase of the succinate of at least 1 percent, particularly in a range of 25 to 537-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a protein phosphatase, or if the activity of the polypeptide At3g06270, preferably represented by SEQ ID NO. 22016, or a homolog or fragment thereof, or if the activity of a polypeptide encoded by a nucleic acid molecule comprising the nucleic acid SEQ ID NO. 22015, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 22015 or polypeptide SEQ ID NO. 22016, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 63 to 203-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a protein phosphatase, or if the activity of the polypeptide At3g06270, preferably represented by SEQ ID NO. 22016, or a homolog or fragment thereof, or if the activity of a polypeptide encoded by a nucleic acid molecule comprising the nucleic acid SEQ ID NO. 22015, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 22015 or polypeptide SEQ ID NO. 22016, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 24 to 55-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a thioredoxin family protein, or if the activity of the polypeptide At3g08710, preferably represented by SEQ ID NO. 22250, or a homolog or fragment thereof, or if the activity of a polypeptide encoded by a nucleic acid molecule comprising the nucleic acid SEQ ID NO. 22249, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 22249 or polypeptide SEQ ID NO. 22250, respectively, or a homolog or a fragment thereof, is increased or generated, or if the activity thioredoxin family protein is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 72 to 165-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a thioredoxin family protein, or if the activity of the polypeptide At3g08710, preferably represented by SEQ ID NO. 22250, or a homolog or fragment thereof, or if the activity of a polypeptide encoded by a nucleic acid molecule comprising the nucleic acid SEQ ID NO. 22249, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 22249 or polypeptide SEQ ID NO. 22250, respectively, or a homolog or a fragment thereof, is increased or generated, or if the activity thioredoxin family protein is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 62 to 162-percent is conferred as compared to a corresponding non-transformed wild type non-human organism.

Accordingly, in one embodiment, an increase of glycerate in a non-human organism, as compared to a corresponding non-transformed wild type non-human organism, is conferred in the process of the invention, if the activity of a polypeptide showing the activity of a thioredoxin, or if the activity of the polypeptide At3g15360, preferably represented by SEQ ID NO. 130492, or a homolog or fragment thereof, or if the activity of a polypeptide encoded by a nucleic acid molecule comprising the nucleic acid SEQ ID NO. 130491, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 130491 or polypeptide SEQ ID NO. 130492, respectively, or a homolog or a fragment thereof, is increased or generated, or if the activity thioredoxin is increased or generated in a non-human organism, like a microorganism or a plant cell, plant or part thereof, especially with non-targeted localization, whereby the respective line disclose in column 7 the fine chemical glycerate. For example, an increase of the glycerate of at least 1 percent, particularly in a range of 31 to 129-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a auxin response factor, or if the activity of the polypeptide At3g61830, preferably represented by SEQ ID NO. 2368, or a homolog or fragment thereof, or if the activity of a polypeptide encoded by a nucleic acid molecule comprising the nucleic acid SEQ ID NO. 2367, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 2367 or polypeptide SEQ ID NO. 2368, respectively, or a homolog or a fragment thereof, is increased or generated, or if the activity auxin response factor is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 78 to 248-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a auxin response factor, or if the activity of the polypeptide At3g61830, preferably represented by SEQ ID NO. 2368, or a homolog or fragment thereof, or if the activity of a polypeptide encoded by a nucleic acid molecule comprising the nucleic acid SEQ ID NO. 2367, preferably the coding region thereof, or a homolog or fragment thereof, e.g. derived from Arabidopsis thaliana, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 2367 or polypeptide SEQ ID NO. 2368, respectively, or a homolog or a fragment thereof, is increased or generated, or if the activity auxin response factor is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 93 to 115-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a monthiol glutaredoxin, or if the activity of the polypeptide At3g62930, preferably represented by SEQ ID NO. 2574, or a homolog or fragment thereof, or if the activity of a polypeptide encoded by a nucleic acid molecule comprising the nucleic acid SEQ ID NO. 2573, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 2573 or polypeptide SEQ ID NO. 2574, respectively, or a homolog or a fragment thereof, is increased or generated, or if the activity monthiol glutaredoxin is increased or generated in a non-human organism, like a microorganism or a plant cell, plant or part thereof, especially with non-targeted localization, whereby the respective line disclose in column 7 the fine chemical citramalate. For example, an increase of the citramalate of at least 1 percent, particularly in a range of 70 to 163-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a monthiol glutaredoxin, or if the activity of the polypeptide At3g62930, preferably represented by SEQ ID NO. 2574, or a homolog or fragment thereof, or if the activity of a polypeptide encoded by a nucleic acid molecule comprising the nucleic acid SEQ ID NO. 2573, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 2573 or polypeptide SEQ ID NO. 2574, respectively, or a homolog or a fragment thereof, is increased or generated, or if the activity monthiol glutaredoxin is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 62 to 222-percent is conferred as compared to a corresponding non-transformed wild type non-human organism.

Accordingly, in one embodiment, an increase of glycerate in a non-human organism, as compared to a corresponding non-transformed wild type non-human organism, is conferred in the process of the invention, if the activity of a polypeptide showing the activity of a monthiol glutaredoxin, or if the activity of the polypeptide At3g62930, preferably represented by SEQ ID NO. 2574, or a homolog or fragment thereof, or if the activity of a polypeptide encoded by a nucleic acid molecule comprising the nucleic acid SEQ ID NO. 2573, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 2573 or polypeptide SEQ ID NO. 2574, respectively, or a homolog or a fragment thereof, is increased or generated, or if the activity monthiol glutaredoxin is increased or generated in a non-human organism, like a microorganism or a plant cell, plant or part thereof, especially with non-targeted localization, whereby the respective line disclose in column 7 the fine chemical glycerate. For example, an increase of the glycerate of at least 1 percent, particularly in a range of 35 to 101-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a monthiol glutaredoxin, or if the activity of the polypeptide At3g62930, preferably represented by SEQ ID NO. 2574, or a homolog or fragment thereof, or if the activity of a polypeptide encoded by a nucleic acid molecule comprising the nucleic acid SEQ ID NO. 2573, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 2573 or polypeptide SEQ ID NO. 2574, respectively, or a homolog or a fragment thereof, is increased or generated, or if the activity monthiol glutaredoxin is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 57 to 206-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a monthiol glutaredoxin, or if the activity of the polypeptide At3g62930, preferably represented by SEQ ID NO. 2574, or a homolog or fragment thereof, or if the activity of a polypeptide encoded by a nucleic acid molecule comprising the nucleic acid SEQ ID NO. 2573, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 2573 or polypeptide SEQ ID NO. 2574, respectively, or a homolog or a fragment thereof, is increased or generated, or if the activity monthiol glutaredoxin is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 30 to 137-percent is conferred as compared to a corresponding non-transformed wild type non-human organism.

Accordingly, in one embodiment, an increase of vitamin c and/or threonic acid in a non-human organism, as compared to a corresponding non-transformed wild type non-human organism, is conferred in the process of the invention, if the activity of a polypeptide showing the activity of a monthiol glutaredoxin, or if the activity of the polypeptide At3g62930, preferably represented by SEQ ID NO. 2574, or a homolog or fragment thereof, or if the activity of a polypeptide encoded by a nucleic acid molecule comprising the nucleic acid SEQ ID NO. 2573, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 2573 or polypeptide SEQ ID NO. 2574, respectively, or a homolog or a fragment thereof, is increased or generated, or if the activity monthiol glutaredoxin is increased or generated in a non-human organism, like a microorganism or a plant cell, plant or part thereof, especially with non-targeted localization, whereby the respective line disclose in column 7 the fine chemical vitamin c and/or threonic acid. For example, an increase of the vitamin c and/or threonic acid of at least 1 percent, particularly in a range of 51 to 96-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical citramalate. For example, an increase of the citramalate of at least 1 percent, particularly in a range of 65 to 235-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 64 to 136-percent is conferred as compared to a corresponding non-transformed wild type non-human organism.

Accordingly, in one embodiment, an increase of glycerate in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical glycerate. For example, an increase of the glycerate of at least 1 percent, particularly in a range of 31 to 113-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 100 to 274-percent is conferred as compared to a corganism responding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 30 to 257-percent is conferred as compared to a corresponding non-transformed wild type non-human organism.

Accordingly, in one embodiment, an increase of glycerate in a non-human organism, as compared to a corresponding non-transformed wild type non-human organism, is conferred in the process of the invention, if the activity of a polypeptide showing the activity of a thioredoxin, or if the activity of the polypeptide At4g03520, preferably represented by SEQ ID NO. 133542, or a homolog or fragment thereof, or if the activity of a polypeptide encoded by a nucleic acid molecule comprising the nucleic acid SEQ ID NO. 133541, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 133541 or polypeptide SEQ ID NO. 133542, respectively, or a homolog or a fragment thereof, is increased or generated, or if the activity thioredoxin is increased or generated in a non-human organism, like a microorganism or a plant cell, plant or part thereof, especially with non-targeted localization, whereby the respective line disclose in column 7 the fine chemical glycerate. For example, an increase of the glycerate of at least 1 percent, particularly in a range of 35 to 112-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical citramalate. For example, an increase of the citramalate of at least 1 percent, particularly in a range of 71 to 156-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 195 to 463-percent is conferred as compared to a corresponding non-transformed wild type non-human organism.

Accordingly, in one embodiment, an increase of glycerate in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical glycerate. For example, an increase of the glycerate of at least 1 percent, particularly in a range of 41 to 158-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 123 to 321-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 133 to 346-percent is conferred as compared to a corresponding non-transformed wild type non-human organism.

Accordingly, in one embodiment, an increase of vitamin c and/or threonic acid in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical vitamin c and/or threonic acid. For example, an increase of the vitamin c and/or threonic acid of at least 1 percent, particularly in a range of 29 to 89-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a monothiol glutaredoxin, or if the activity of the polypeptide At4g15670, preferably represented by SEQ ID NO. 3280, or a homolog or fragment thereof, or if the activity of a polypeptide encoded by a nucleic acid molecule comprising the nucleic acid SEQ ID NO. 3279, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 3279 or polypeptide SEQ ID NO. 3280, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 196 to 830-percent is conferred as compared to a corresponding non-transformed wild type non-human organism.

Accordingly, in one embodiment, an increase of glycerate in a non-human organism, as compared to a corresponding non-transformed wild type non-human organism, is conferred in the process of the invention, if the activity of a polypeptide showing the activity of a monothiol glutaredoxin, or if the activity of the polypeptide At4g15670, preferably represented by SEQ ID NO. 3280, or a homolog or fragment thereof, or if the activity of a polypeptide encoded by a nucleic acid molecule comprising the nucleic acid SEQ ID NO. 3279, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 3279 or polypeptide SEQ ID NO. 3280, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical glycerate. For example, an increase of the glycerate of at least 1 percent, particularly in a range of 49 to 224-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a monothiol glutaredoxin, or if the activity of the polypeptide At4g15670, preferably represented by SEQ ID NO. 3280, or a homolog or fragment thereof, or if the activity of a polypeptide encoded by a nucleic acid molecule comprising the nucleic acid SEQ ID NO. 3279, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 3279 or polypeptide SEQ ID NO. 3280, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 117 to 935-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a monothiol glutaredoxin, or if the activity of the polypeptide At4g15670, preferably represented by SEQ ID NO. 3280, or a homolog or fragment thereof, or if the activity of a polypeptide encoded by a nucleic acid molecule comprising the nucleic acid SEQ ID NO. 3279, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 3279 or polypeptide SEQ ID NO. 3280, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 77 to 314-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a monothiol glutaredoxin, or if the activity of the polypeptide At4g15690, preferably represented by SEQ ID NO. 23845, or a homolog or fragment thereof, or if the activity of a polypeptide encoded by a nucleic acid molecule comprising the nucleic acid SEQ ID NO. 23844, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 23844 or polypeptide SEQ ID NO. 23845, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical citramalate. For example, an increase of the citramalate of at least 1 percent, particularly in a range of 47 to 204-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a monothiol glutaredoxin, or if the activity of the polypeptide At4g15690, preferably represented by SEQ ID NO. 23845, or a homolog or fragment thereof, or if the activity of a polypeptide encoded by a nucleic acid molecule comprising the nucleic acid SEQ ID NO. 23844, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 23844 or polypeptide SEQ ID NO. 23845, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 66 to 427-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a monothiol glutaredoxin, or if the activity of the polypeptide At4g15690, preferably represented by SEQ ID NO. 23845, or a homolog or fragment thereof, or if the activity of a polypeptide encoded by a nucleic acid molecule comprising the nucleic acid SEQ ID NO. 23844, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 23844 or polypeptide SEQ ID NO. 23845, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 76 to 386-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a monothiol glutaredoxin, or if the activity of the polypeptide At4g15690, preferably represented by SEQ ID NO. 23845, or a homolog or fragment thereof, or if the activity of a polypeptide encoded by a nucleic acid molecule comprising the nucleic acid SEQ ID NO. 23844, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 23844 or polypeptide SEQ ID NO. 23845, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 32 to 158-percent is conferred as compared to a corresponding non-transformed wild type non-human organism.

Accordingly, in one embodiment, an increase of vitamin c and/or threonic acid in a non-human organism, as compared to a corresponding non-transformed wild type non-human organism, is conferred in the process of the invention, if the activity of a polypeptide showing the activity of a monothiol glutaredoxin, or if the activity of the polypeptide At4g15690, preferably represented by SEQ ID NO. 23845, or a homolog or fragment thereof, or if the activity of a polypeptide encoded by a nucleic acid molecule comprising the nucleic acid SEQ ID NO. 23844, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 23844 or polypeptide SEQ ID NO. 23845, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical vitamin c and/or threonic acid. For example, an increase of the vitamin c and/or threonic acid of at least 1 percent, particularly in a range of 30 to 86-percent is conferred as compared to a corresponding non-transformed wild type non-human organism.

Accordingly, in one embodiment, an increase of glycerate in a non-human organism, as compared to a corresponding non-transformed wild type non-human organism, is conferred in the process of the invention, if the activity of a polypeptide showing the activity of a monothiol glutaredoxin, or if the activity of the polypeptide At4g15700, preferably represented by SEQ ID NO. 3655, or a homolog or fragment thereof, or if the activity of a polypeptide encoded by a nucleic acid molecule comprising the nucleic acid SEQ ID NO. 3654, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 3654 or polypeptide SEQ ID NO. 3655, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical glycerate. For example, an increase of the glycerate of at least 1 percent, particularly in a range of 48 to 198-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a protein phosphatase, or if the activity of the polypeptide At4g26080, preferably represented by SEQ ID NO. 68850, or a homolog or fragment thereof, or if the activity of a polypeptide encoded by a nucleic acid molecule comprising the nucleic acid SEQ ID NO. 68849, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 68849 or polypeptide SEQ ID NO. 68850, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical citramalate. For example, an increase of the citramalate of at least 1 percent, particularly in a range of 47 to 70-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a protein phosphatase, or if the activity of the polypeptide At4g26080, preferably represented by SEQ ID NO. 68850, or a homolog or fragment thereof, or if the activity of a polypeptide encoded by a nucleic acid molecule comprising the nucleic acid SEQ ID NO. 68849, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 68849 or polypeptide SEQ ID NO. 68850, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 30 to 52-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a glutaredoxin, or if the activity of the polypeptide At4g33040, preferably represented by SEQ ID NO. 4103, or a homolog or fragment thereof, or if the activity of a polypeptide encoded by a nucleic acid molecule comprising the nucleic acid SEQ ID NO. 4102, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 4102 or polypeptide SEQ ID NO. 4103, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical citramalate. For example, an increase of the citramalate of at least 1 percent, particularly in a range of 43 to 136-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a glutaredoxin, or if the activity of the polypeptide At4g33040, preferably represented by SEQ ID NO. 4103, or a homolog or fragment thereof, or if the activity of a polypeptide encoded by a nucleic acid molecule comprising the nucleic acid SEQ ID NO. 4102, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 4102 or polypeptide SEQ ID NO. 4103, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 72 to 537-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a glutaredoxin, or if the activity of the polypeptide At4g33040, preferably represented by SEQ ID NO. 4103, or a homolog or fragment thereof, or if the activity of a polypeptide encoded by a nucleic acid molecule comprising the nucleic acid SEQ ID NO. 4102, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 4102 or polypeptide SEQ ID NO. 4103, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 65 to 296-percent is conferred as compared to a corganism responding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a glutaredoxin, or if the activity of the polypeptide At4g33040, preferably represented by SEQ ID NO. 4103, or a homolog or fragment thereof, or if the activity of a polypeptide encoded by a nucleic acid molecule comprising the nucleic acid SEQ ID NO. 4102, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 4102 or polypeptide SEQ ID NO. 4103, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 42 to 149-percent is conferred as compared to a corresponding non-transformed wild type non-human organism.

Accordingly, in one embodiment, an increase of vitamin c and/or threonic acid in a non-human organism, as compared to a corresponding non-transformed wild type non-human organism, is conferred in the process of the invention, if the activity of a polypeptide showing the activity of a glutaredoxin, or if the activity of the polypeptide At4g33040, preferably represented by SEQ ID NO. 4103, or a homolog or fragment thereof, or if the activity of a polypeptide encoded by a nucleic acid molecule comprising the nucleic acid SEQ ID NO. 4102, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 4102 or polypeptide SEQ ID NO. 4103, respectively, or a homolog or a fragment thereof, is increased or generated, or if the activity glutaredoxin is increased or generated in a non-human organism, like a microorganism organism or a plant cell, plant or part thereof, especially with non-targeted localization, whereby the respective line disclose in column 7 the fine chemical vitamin c and/or threonic acid. For example, an increase of the vitamin c and/or threonic acid of at least 1 percent, particularly in a range of 32 to 102-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 70 to 296-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a heat shock transcription factor, or if the activity of the polypeptide At5g03720, preferably represented by SEQ ID NO. 24439, or a homolog or fragment thereof, or if the activity of a polypeptide encoded by a nucleic acid molecule comprising the nucleic acid SEQ ID NO. 24438, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 24438 or polypeptide SEQ ID NO. 24439, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 66 to 1135-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a heat shock transcription factor, or if the activity of the polypeptide At5g03720, preferably represented by SEQ ID NO. 24439, or a homolog or fragment thereof, or if the activity of a polypeptide encoded by a nucleic acid molecule comprising the nucleic acid SEQ ID NO. 24438, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 24438 or polypeptide SEQ ID NO. 24439, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 133 to 687-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a heat shock transcription factor, or if the activity of the polypeptide At5g03720, preferably represented by SEQ ID NO. 24439, or a homolog or fragment thereof, or if the activity of a polypeptide encoded by a nucleic acid molecule comprising the nucleic acid SEQ ID NO. 24438, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 24438 or polypeptide SEQ ID NO. 24439, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 30 to 376-percent is conferred as compared to a corresponding non-transformed wild type non-human organism.

Accordingly, in one embodiment, an increase of glycerate in a non-human organism, as compared to a corresponding non-transformed wild type non-human organism, is conferred in the process of the invention, if the activity of a polypeptide showing the activity of a gibberellin 20-oxidase, or if the activity of the polypeptide At5g07200, preferably represented by SEQ ID NO. 24493, or a homolog or fragment thereof, or if the activity of a polypeptide encoded by a nucleic acid molecule comprising the nucleic acid SEQ ID NO. 24492, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 24492 or polypeptide SEQ ID NO. 24493, respectively, or a homolog or a fragment thereof, is increased or generated, or if the activity gibberellin 20-oxidase is increased or generated in a non-human organism, like a microorganism or a plant cell, plant or part thereof, especially with non-targeted localization, whereby the respective line disclose in column 7 the fine chemical glycerate. For example, an increase of the glycerate of at least 1 percent, particularly in a range of 33 to 74-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a gibberellin 20-oxidase, or if the activity of the polypeptide At5g07200, preferably represented by SEQ ID NO. 24493, or a homolog or fragment thereof, or if the activity of a polypeptide encoded by a nucleic acid molecule comprising the nucleic acid SEQ ID NO. 24492, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 24492 or polypeptide SEQ ID NO. 24493, respectively, or a homolog or a fragment thereof, is increased or generated, or if the activity gibberellin 20-oxidase is increased or generated in a non-human organism, like a microorganism organism or a plant cell, plant or part thereof, especially with non-targeted localization, whereby the respective line disclose in column 7 the fine chemical malate. For example, an increase of the malate of at least 1 percent, particularly in a range of 52 to 329-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a monothiol glutaredoxin, or if the activity of the polypeptide At5g18600, preferably represented by SEQ ID NO. 4905, or a homolog or fragment thereof, or if the activity of a polypeptide encoded by a nucleic acid molecule comprising the nucleic acid SEQ ID NO. 4904, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 4904 or polypeptide SEQ ID NO. 4905, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 39 to 132-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a translation initiation factor subunit, or if the activity of the polypeptide At5g27640, preferably represented by SEQ ID NO. 25345, or a homolog or fragment thereof, or if the activity of a polypeptide encoded by a nucleic acid molecule comprising the nucleic acid SEQ ID NO. 25344, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 25344 or polypeptide SEQ ID NO. 25345, respectively, or a homolog or a fragment thereof, is increased or generated, or if the activity translation initiation factor subunit is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 52 to 173-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a transcription factor, or if the activity of the polypeptide At5g39760, preferably represented by SEQ ID NO. 25429, or a homolog or fragment thereof, or if the activity of a polypeptide encoded by a nucleic acid molecule comprising the nucleic acid SEQ ID NO. 25428, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 25428 or polypeptide SEQ ID NO. 25429, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 54 to 110-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a transcription factor, or if the activity of the polypeptide At5g39760, preferably represented by SEQ ID NO. 25429, or a homolog or fragment thereof, or if the activity of a polypeptide encoded by a nucleic acid molecule comprising the nucleic acid SEQ ID NO. 25428, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 25428 or polypeptide SEQ ID NO. 25429, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 52 to 175-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a protein phosphatase, or if the activity of the polypeptide At5g59220, preferably represented by SEQ ID NO. 25499, or a homolog or fragment thereof, or if the activity of a polypeptide encoded by a nucleic acid molecule comprising the nucleic acid SEQ ID NO. 25498, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 25498 or polypeptide SEQ ID NO. 25499, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 30 to 67-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a pyruvate kinase, or if the activity of the polypeptide At5g63680, preferably represented by SEQ ID NO. 70039, or a homolog or fragment thereof, or if the activity of a polypeptide encoded by a nucleic acid molecule comprising the nucleic acid SEQ ID NO. 70038, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 70038 or polypeptide SEQ ID NO. 70039, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line disclose in column 7 the fine chemical succinate. For example, an increase of the succinate of at least 1 percent, particularly in a range of 31 to 226-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a zinc finger protein, or if the activity of the polypeptide At5g64920, preferably represented by SEQ ID NO. 5494, or a homolog or fragment thereof, or if the activity of a polypeptide encoded by a nucleic acid molecule comprising the nucleic acid SEQ ID NO. 5493, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 5493 or polypeptide SEQ ID NO. 5494, respectively, or a homolog or a fragment thereof, is increased or generated, or if the activity zinc finger protein is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 81 to 158-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a CDP-diacylglycerol-serine O-phosphatidyltransferase, or if the activity of the polypeptide AvinDRAFT_{—}0035, preferably represented by SEQ ID NO. 70584, or a homolog or fragment thereof, or if the activity of a polypeptide encoded by a nucleic acid molecule comprising the nucleic acid SEQ ID NO. 70583, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 70583 or polypeptide SEQ ID NO. 70584, respectively, or a homolog or a fragment thereof, is increased or generated, or if the activity CDP-diacylglycerol-serine O-phosphatidyltransferase is increased or generated in a non-human organism, like a microorganism or a plant cell, plant or part thereof, especially with non-targeted localization, whereby the respective line disclose in column 7 the fine chemical citramalate. For example, an increase of the citramalate of at least 1 percent, particularly in a range of 41 to 106-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a CDP-diacylglycerol-serine O-phosphatidyltransferase, or if the activity of the polypeptide AvinDRAFT_{—}0035, preferably represented by SEQ ID NO. 70584, or a homolog or fragment thereof, or if the activity of a polypeptide encoded by a nucleic acid molecule comprising the nucleic acid SEQ ID NO. 70583, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 70583 or polypeptide SEQ ID NO. 70584, respectively, or a homolog or a fragment thereof, is increased or generated, or if the activity CDP-diacylglycerol-serine O-phosphatidyltransferase is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 63 to 130-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a CDP-diacylglycerolserine O-phosphatidyltransferase, or if the activity of the polypeptide AvinDRAFT_{—}0035, preferably represented by SEQ ID NO. 70584, or a homolog or fragment thereof, or if the activity of a polypeptide encoded by a nucleic acid molecule comprising the nucleic acid SEQ ID NO. 70583, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 70583 or polypeptide SEQ ID NO. 70584, respectively, or a homolog or a fragment thereof, is increased or generated, or if the activity CDP-diacylglycerol-serine O-phosphatidyltransferase is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 55 to 281-percent is conferred as compared to a corganism responding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a Glycine cleavage system T aminomethyltransferase, or if the activity of the polypeptide AVINDRAFT_{—}0539, preferably represented by SEQ ID NO. 70716, or a homolog or fragment thereof, or if the activity of a polypeptide encoded by a nucleic acid molecule comprising the nucleic acid SEQ ID NO. 70715, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 70715 or polypeptide SEQ ID NO. 70716, respectively, or a homolog or a fragment thereof, is increased or generated, or if the activity Glycine cleavage system T aminomethyltransferase is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 52 to 146-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a transcriptional regulator, or if the activity of the polypeptide AvinDRAFT_{—}1045, preferably represented by SEQ ID NO. 25677, or a homolog or fragment thereof, or if the activity of a polypeptide encoded by a nucleic acid molecule comprising the nucleic acid SEQ ID NO. 25676, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 25676 or polypeptide SEQ ID NO. 25677, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 64 to 122-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a adenylylsulfate kinase, or if the activity of the polypeptide AvinDRAFT_{—}1398, preferably represented by SEQ ID NO. 25781, or a homolog or fragment thereof, or if the activity of a polypeptide encoded by a nucleic acid molecule comprising the nucleic acid SEQ ID NO. 25780, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 25780 or polypeptide SEQ ID NO. 25781, respectively, or a homolog or a fragment thereof, is increased or generated, or if the activity adenylylsulfate kinase is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 54 to 91-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a adenylylsulfate kinase, or if the activity of the polypeptide AvinDRAFT_{—}1398, preferably represented by SEQ ID NO. 25781, or a homolog or fragment thereof, or if the activity of a polypeptide encoded by a nucleic acid molecule comprising the nucleic acid SEQ ID NO. 25780, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 25780 or polypeptide SEQ ID NO. 25781, respectively, or a homolog or a fragment thereof, is increased or generated, or if the activity adenylylsulfate kinase is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 32 to 90-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a sec-independent protein translocase, or if the activity of the polypeptide AvinDRAFT_{—}1624, preferably represented by SEQ ID NO. 26197, or a homolog or fragment thereof, or if the activity of a polypeptide encoded by a nucleic acid molecule comprising the nucleic acid SEQ ID NO. 26196, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 26196 or polypeptide SEQ ID NO. 26197, respectively, or a homolog or a fragment thereof, is increased or generated, or if the activity sec-independent protein translocase is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 66 to 110-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a sec-independent protein translocase, or if the activity of the polypeptide AvinDRAFT_{—}1624, preferably represented by SEQ ID NO. 26197, or a homolog or fragment thereof, or if the activity of a polypeptide encoded by a nucleic acid molecule comprising the nucleic acid SEQ ID NO. 26196, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 26196 or polypeptide SEQ ID NO. 26197, respectively, or a homolog or a fragment thereof, is increased or generated, or if the activity sec-independent protein translocase is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 67 to 120-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a fumarate hydratase, or if the activity of the polypeptide AvinDRAFT_{—}1699, preferably represented by SEQ ID NO. 133720, or a homolog or fragment thereof, or if the activity of a polypeptide encoded by a nucleic acid molecule comprising the nucleic acid SEQ ID NO. 133719, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 133719 or polypeptide SEQ ID NO. 133720, respectively, or a homolog or a fragment thereof, is increased or generated, or if the activity fumarate hydratase is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 148 to 507-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a pyruvate kinase, or if the activity of the polypeptide AvinDRAFT_{—}1806, preferably represented by SEQ ID NO. 26435, or a homolog or fragment thereof, or if the activity of a polypeptide encoded by a nucleic acid molecule comprising the nucleic acid SEQ ID NO. 26434, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 26434 or polypeptide SEQ ID NO. 26435, respectively, or a homolog or a fragment thereof, is increased or generated, or if the activity pyruvate kinase is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 51 to 154-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a transcriptional regulator protein, or if the activity of the polypeptide AvinDRAFT_{—}2010, preferably represented by SEQ ID NO. 114199, or a homolog or fragment thereof, or if the activity of a polypeptide encoded by a nucleic acid molecule comprising the nucleic acid SEQ ID NO. 114198, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 114198 or polypeptide SEQ ID NO. 114199, respectively, or a homolog or a fragment thereof, is increased or generated, or if the activity transcriptional regulator protein is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 30 to 43-percent is conferred as compared to a corresponding non-transformed wild type non-human organism.

Accordingly, in one embodiment, an increase of glycerate in a non-human organism, as compared to a corresponding non-transformed wild type non-human organism, is conferred in the process of the invention, if the activity of a polypeptide showing the activity of a beta-hydroxylase, or if the activity of the polypeptide AvinDRAFT_{—}2091, preferably represented by SEQ ID NO. 6041, or a homolog or fragment thereof, or if the activity of a polypeptide encoded by a nucleic acid molecule comprising the nucleic acid SEQ ID NO. 6040, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 6040 or polypeptide SEQ ID NO. 6041, respectively, or a homolog or a fragment thereof, is increased or generated, or if the activity beta-hydroxylase is increased or generated in a non-human organism, like a microorganism or a plant cell, plant or part thereof, especially with non-targeted localization, whereby the respective line disclose in column 7 the fine chemical glycerate. For example, an increase of the glycerate of at least 1 percent, particularly in a range of 31 to 81-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a 30S ribosomal protein S3, or if the activity of the polypeptide AvinDRAFT_{—}2365, preferably represented by SEQ ID NO. 71326, or a homolog or fragment thereof, or if the activity of a polypeptide encoded by a nucleic acid molecule comprising the nucleic acid SEQ ID NO. 71325, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 71325 or polypeptide SEQ ID NO. 71326, respectively, or a homolog or a fragment thereof, is increased or generated, or if the activity 30S ribosomal protein S3 is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 45 to 203-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a 50S ribosomal protein L14, or if the activity of the polypeptide AvinDRAFT_{—}2369, preferably represented by SEQ ID NO. 71764, or a homolog or fragment thereof, or if the activity of a polypeptide encoded by a nucleic acid molecule comprising the nucleic acid SEQ ID NO. 71763, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 71763 or polypeptide SEQ ID NO. 71764, respectively, or a homolog or a fragment thereof, is increased or generated, or if the activity 50S ribosomal protein L14 is increased or generated in a non-human organism, like a microorganism or a plant cell, plant or part thereof, especially with non-targeted localization, whereby the respective line disclose in column 7 the fine chemical citramalate. For example, an increase of the citramalate of at least 1 percent, particularly in a range of 41 to 49-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a acyl-CoA synthase, or if the activity of the polypeptide AvinDRAFT_{—}2754, preferably represented by SEQ ID NO. 28041, or a homolog or fragment thereof, or if the activity of a polypeptide encoded by a nucleic acid molecule comprising the nucleic acid SEQ ID NO. 28040, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 28040 or polypeptide SEQ ID NO. 28041, respectively, or a homolog or a fragment thereof, is increased or generated, or if the activity acyl-CoA synthase is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 121 to 517-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a 2-oxoglutarate dehydrogenase E1 subunit, or if the activity of the polypeptide AvinDRAFT_{—}3028, preferably represented by SEQ ID NO. 6076, or a homolog or fragment thereof, or if the activity of a polypeptide encoded by a nucleic acid molecule comprising the nucleic acid SEQ ID NO. 6075, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 6075 or polypeptide SEQ ID NO. 6076, respectively, or a homolog or a fragment thereof, is increased or generated, or if the activity 2-oxoglutarate dehydrogenase E1 subunit is increased or generated in a non-human organism, like a microorganism or a plant cell, plant or part thereof, especially with non-targeted localization, whereby the respective line disclose in column 7 the fine chemical citramalate. For example, an increase of the citramalate of at least 1 percent, particularly in a range of 47 to 117-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a 2-oxoglutarate dehydrogenase E1 subunit, or if the activity of the polypeptide AvinDRAFT_{—}3028, preferably represented by SEQ ID NO. 6076, or a homolog or fragment thereof, or if the activity of a polypeptide encoded by a nucleic acid molecule comprising the nucleic acid SEQ ID NO. 6075, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 6075 or polypeptide SEQ ID NO. 6076, respectively, or a homolog or a fragment thereof, is increased or generated, or if the activity 2-oxoglutarate dehydrogenase E1 subunit is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 98 to 406-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a 2-oxoglutarate dehydrogenase E1 subunit, or if the activity of the polypeptide AvinDRAFT_{—}3028, preferably represented by SEQ ID NO. 6076, or a homolog or fragment thereof, or if the activity of a polypeptide encoded by a nucleic acid molecule comprising the nucleic acid SEQ ID NO. 6075, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 6075 or polypeptide SEQ ID NO. 6076, respectively, or a homolog or a fragment thereof, is increased or generated, or if the activity 2-oxoglutarate dehydrogenase E1 subunit is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 57 to 323-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a 2-oxoglutarate dehydrogenase E1 subunit, or if the activity of the polypeptide AvinDRAFT_{—}3028, preferably represented by SEQ ID NO. 6076, or a homolog or fragment thereof, or if the activity of a polypeptide encoded by a nucleic acid molecule comprising the nucleic acid SEQ ID NO. 6075, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 6075 or polypeptide SEQ ID NO. 6076, respectively, or a homolog or a fragment thereof, is increased or generated, or if the activity 2-oxoglutarate dehydrogenase E1 subunit is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 97 to 190-percent is conferred as compared to a corresponding non-transformed wild type non-human organism.

Accordingly, in one embodiment, an increase of vitamin c and/or threonic acid in a non-human organism, as compared to a corresponding non-transformed wild type non-human organism, is conferred in the process of the invention, if the activity of a polypeptide showing the activity of a 2-oxoglutarate dehydrogenase E1 subunit, or if the activity of the polypeptide AvinDRAFT_{—}3028, preferably represented by SEQ ID NO. 6076, or a homolog or fragment thereof, or if the activity of a polypeptide encoded by a nucleic acid molecule comprising the nucleic acid SEQ ID NO. 6075, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 6075 or polypeptide SEQ ID NO. 6076, respectively, or a homolog or a fragment thereof, is increased or generated, or if the activity 2-oxoglutarate dehydrogenase E1 subunit is increased or generated in a non-human organism, like a microorganism or a plant cell, plant or part thereof, especially with non-targeted localization, whereby the respective line disclose in column 7 the fine chemical vitamin c and/or threonic acid. For example, an increase of the vitamin c and/or threonic acid of at least 1 percent, particularly in a range of 31 to 137-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a transcriptional regulator, or if the activity of the polypeptide AvinDRAFT_{—}3186, preferably represented by SEQ ID NO. 29247, or a homolog or fragment thereof, or if the activity of a polypeptide encoded by a nucleic acid molecule comprising the nucleic acid SEQ ID NO. 29246, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 29246 or polypeptide SEQ ID NO. 29247, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line disclose in column 7 the fine chemical citramalate. For example, an increase of the citramalate of at least 1 percent, particularly in a range of 41 to 84-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a transcriptional regulator, or if the activity of the polypeptide AvinDRAFT_{—}3186, preferably represented by SEQ ID NO. 29247, or a homolog or fragment thereof, or if the activity of a polypeptide encoded by a nucleic acid molecule comprising the nucleic acid SEQ ID NO. 29246, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 29246 or polypeptide SEQ ID NO. 29247, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 100 to 236-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a oxidoreductase, or if the activity of the polypeptide AvinDRAFT_{—}3253, preferably represented by SEQ ID NO. 29501, or a homolog or fragment thereof, or if the activity of a polypeptide encoded by a nucleic acid molecule comprising the nucleic acid SEQ ID NO. 29500, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 29500 or polypeptide SEQ ID NO. 29501, respectively, or a homolog or a fragment thereof, is increased or generated, or if the activity oxidoreductase is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 29 to 139-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a CTP synthase, or if the activity of the polypeptide AvinDRAFT_{—}3605, preferably represented by SEQ ID NO. 31027, or a homolog or fragment thereof, or if the activity of a polypeptide encoded by a nucleic acid molecule comprising the nucleic acid SEQ ID NO. 31026, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 31026 or polypeptide SEQ ID NO. 31027, respectively, or a homolog or a fragment thereof, is increased or generated, or if the activity CTP synthase is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 61 to 240-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a CTP synthase, or if the activity of the polypeptide AvinDRAFT_{—}3605, preferably represented by SEQ ID NO. 31027, or a homolog or fragment thereof, or if the activity of a polypeptide encoded by a nucleic acid molecule comprising the nucleic acid SEQ ID NO. 31026, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 31026 or polypeptide SEQ ID NO. 31027, respectively, or a homolog or a fragment thereof, is increased or generated, or if the activity CTP synthase is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 53 to 314-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a CTP synthase, or if the activity of the polypeptide AvinDRAFT_{—}3605, preferably represented by SEQ ID NO.

31027, or a homolog or fragment thereof, or if the activity of a polypeptide encoded by a nucleic acid molecule comprising the nucleic acid SEQ ID NO. 31026, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 31026 or polypeptide SEQ ID NO. 31027, respectively, or a homolog or a fragment thereof, is increased or generated, or if the activity CTP synthase is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 66 to 203-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a recombinase A, or if the activity of the polypeptide AvinDRAFT_{—}3629, preferably represented by SEQ ID NO. 114232, or a homolog or fragment thereof, or if the activity of a polypeptide encoded by a nucleic acid molecule comprising the nucleic acid SEQ ID NO. 114231, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 114231 or polypeptide SEQ ID NO. 114232, respectively, or a homolog or a fragment thereof, is increased or generated, or if the activity recombinase A is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 33 to 291-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a glucose-1-phosphate cytidylyltransferase, or if the activity of the polypeptide AvinDRAFT_{—}4384, preferably represented by SEQ ID NO. 31718, or a homolog or fragment thereof, or if the activity of a polypeptide encoded by a nucleic acid molecule comprising the nucleic acid SEQ ID NO. 31717, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 31717 or polypeptide SEQ ID NO. 31718, respectively, or a homolog or a fragment thereof, is increased or generated, or if the activity glucose-1-phosphate cytidylyltransferase is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 73 to 137-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a CDP-glucose-4,6-dehydratase, or if the activity of the polypeptide AvinDRAFT_{—}4385, preferably represented by SEQ ID NO. 72936, or a homolog or fragment thereof, or if the activity of a polypeptide encoded by a nucleic acid molecule comprising the nucleic acid SEQ ID NO. 72935, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 72935 or polypeptide SEQ ID NO. 72936, respectively, or a homolog or a fragment thereof, is increased or generated, or if the activity CDP-glucose-4,6-dehydratase is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 57 to 91-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a CDP-glucose-4,6-dehydratase, or if the activity of the polypeptide AvinDRAFT_{—}4385, preferably represented by SEQ ID NO. 72936, or a homolog or fragment thereof, or if the activity of a polypeptide encoded by a nucleic acid molecule comprising the nucleic acid SEQ ID NO. 72935, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 72935 or polypeptide SEQ ID NO. 72936, respectively, or a homolog or a fragment thereof, is increased or generated, or if the activity CDP-glucose-4,6-dehydratase is increased or generated in a non-human organism, like a microorganism or a plant cell, plant or part thereof, especially with non-targeted localization, whereby the respective line disclose in column 7 the fine chemical malate.

For example, an increase of the malate of at least 1 percent, particularly in a range of 82 to 144-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a aminotransferase, or if the activity of the polypeptide AvinDRAFT_{—}4562, preferably represented by SEQ ID NO. 31927, or a homolog or fragment thereof, or if the activity of a polypeptide encoded by a nucleic acid molecule comprising the nucleic acid SEQ ID NO. 31926, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 31926 or polypeptide SEQ ID NO. 31927, respectively, or a homolog or a fragment thereof, is increased or generated, or if the activity aminotransferase is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 59 to 109-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a isocitrate lyase, or if the activity of the polypeptide AvinDRAFT_{—}4594, preferably represented by SEQ ID NO. 134172, or a homolog or fragment thereof, or if the activity of a polypeptide encoded by a nucleic acid molecule comprising the nucleic acid SEQ ID NO. 134171, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 134171 or polypeptide SEQ ID NO. 134172, respectively, or a homolog or a fragment thereof, is increased or generated, or if the activity isocitrate lyase is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 33 to 110-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a NADH-quinone oxidoreductase subunit, or if the activity of the polypeptide AvinDRAFT_{—}4606, preferably represented by SEQ ID NO. 73720, or a homolog or fragment thereof, or if the activity of a polypeptide encoded by a nucleic acid molecule comprising the nucleic acid SEQ ID NO. 73719, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 73719 or polypeptide SEQ ID NO. 73720, respectively, or a homolog or a fragment thereof, is increased or generated, or if the activity NADH-quinone oxidoreductase subunit is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 52 to 113-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a NADH-quinone oxidoreductase subunit, or if the activity of the polypeptide AvinDRAFT_{—}4606, preferably represented by SEQ ID NO. 73720, or a homolog or fragment thereof, or if the activity of a polypeptide encoded by a nucleic acid molecule comprising the nucleic acid SEQ ID NO. 73719, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 73719 or polypeptide SEQ ID NO. 73720, respectively, or a homolog or a fragment thereof, is increased or generated, or if the activity NADH-quinone oxidoreductase subunit is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 70 to 81-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a RNA polymerase sigma factor, or if the activity of the polypeptide AvinDRAFT_{—}4989, preferably represented by SEQ ID NO. 74453, or a homolog or fragment thereof, or if the activity of a polypeptide encoded by a nucleic acid molecule comprising the nucleic acid SEQ ID NO. 74452, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 74452 or polypeptide SEQ ID NO. 74453, respectively, or a homolog or a fragment thereof, is increased or generated, or if the activity RNA polymerase sigma factor is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 59 to 107-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a hydrolase, or if the activity of the polypeptide AvinDRAFT_{—}5103, preferably represented by SEQ ID NO. 6511, or a homolog or fragment thereof, or if the activity of a polypeptide encoded by a nucleic acid molecule comprising the nucleic acid SEQ ID NO. 6510, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 6510 or polypeptide SEQ ID NO. 6511, respectively, or a homolog or a fragment thereof, is increased or generated, or if the activity hydrolase is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 74 to 412-percent is conferred as compared to a corganism responding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a glutamate-ammonia-ligase, or if the activity of the polypeptide AvinDRAFT_{—}5246, preferably represented by SEQ ID NO. 32038, or a homolog or fragment thereof, or if the activity of a polypeptide encoded by a nucleic acid molecule comprising the nucleic acid SEQ ID NO. 32037, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 32037 or polypeptide SEQ ID NO. 32038, respectively, or a homolog or a fragment thereof, is increased or generated, or if the activity glutamate-ammonia-ligase is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 181 to 293-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a electron transfer flavoprotein subunit beta, or if the activity of the polypeptide AvinDRAFT_{—}6679, preferably represented by SEQ ID NO. 74730, or a homolog or fragment thereof, or if the activity of a polypeptide encoded by a nucleic acid molecule comprising the nucleic acid SEQ ID NO. 74729, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 74729 or polypeptide SEQ ID NO. 74730, respectively, or a homolog or a fragment thereof, is increased or generated, or if the activity electron transfer flavoprotein subunit beta is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 26 to 107-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a AX653549-protein, or if the activity of the polypeptide AX653549, preferably represented by SEQ ID NO. 34302, or a homolog or fragment thereof, or if the activity of a polypeptide encoded by a nucleic acid molecule comprising the nucleic acid SEQ ID NO. 34301, preferably the coding region thereof, or a homolog or fragment thereof, e.g. derived from Oryza sativa, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 34301 or polypeptide SEQ ID NO. 34302, respectively, or a homolog or a fragment thereof, is increased or generated, or if the activity AX653549-protein is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 92 to 266-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a AX653549-protein, or if the activity of the polypeptide AX653549, preferably represented by SEQ ID NO. 34302, or a homolog or fragment thereof, or if the activity of a polypeptide encoded by a nucleic acid molecule comprising the nucleic acid SEQ ID NO. 34301, preferably the coding region thereof, or a homolog or fragment thereof, e.g. derived from Oryza sativa, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 34301 or polypeptide SEQ ID NO. 34302, respectively, or a homolog or a fragment thereof, is increased or generated, or if the activity AX653549-protein is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 62 to 106-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a AY087308-protein, or if the activity of the polypeptide AY087308, preferably represented by SEQ ID NO.

34603, or a homolog or fragment thereof, or if the activity of a polypeptide encoded by a nucleic acid molecule comprising the nucleic acid SEQ ID NO. 34602, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 34602 or polypeptide SEQ ID NO. 34603, respectively, or a homolog or a fragment thereof, is increased or generated, or if the activity AY087308-protein is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 84 to 224-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a AY087308-protein, or if the activity of the polypeptide AY087308, preferably represented by SEQ ID NO. 34603, or a homolog or fragment thereof, or if the activity of a polypeptide encoded by a nucleic acid molecule comprising the nucleic acid SEQ ID NO. 34602, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 34602 or polypeptide SEQ ID NO. 34603, respectively, or a homolog or a fragment thereof, is increased or generated, or if the activity AY087308-protein is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 51 to 252-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a AY087308-protein, or if the activity of the polypeptide AY087308, preferably represented by SEQ ID NO. 34603, or a homolog or fragment thereof, or if the activity of a polypeptide encoded by a nucleic acid molecule comprising the nucleic acid SEQ ID NO. 34602, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 34602 or polypeptide SEQ ID NO. 34603, respectively, or a homolog or a fragment thereof, is increased or generated, or if the activity AY087308-protein is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 33 to 109-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a fumarate hydratase, or if the activity of the polypeptide Bab08741, preferably represented by SEQ ID NO. 136121, or a homolog or fragment thereof, or if the activity of a polypeptide encoded by a nucleic acid molecule comprising the nucleic acid SEQ ID NO. 136120, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 136120 or polypeptide SEQ ID NO. 136121, respectively, or a homolog or a fragment thereof, is increased or generated, or if the activity fumarate hydratase is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 165 to 582-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a threonine synthase, or if the activity of the polypeptide B0004, preferably represented by SEQ ID NO. 34890, or a homolog or fragment thereof, or if the activity of a polypeptide encoded by a nucleic acid molecule comprising the nucleic acid SEQ ID NO. 34889, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 34889 or polypeptide SEQ ID NO. 34890, respectively, or a homolog or a fragment thereof, is increased or generated, or if the activity threonine synthase is increased or generated in a non-human organism, like a microorganism or a plant cell, plant or part thereof, especially with plastidic localization, whereby the respective line disclose in column 7 the fine chemical malate. For example, an increase of the malate of at least 1 percent, particularly in a range of 61 to 169-percent is conferred as compared to a corganism responding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a ribulokinase, or if the activity of the polypeptide B0063, preferably represented by SEQ ID NO. 134300, or a homolog or fragment thereof, or if the activity of a polypeptide encoded by a nucleic acid molecule comprising the nucleic acid SEQ ID NO. 134299, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 134299 or polypeptide SEQ ID NO. 134300, respectively, or a homolog or a fragment thereof, is increased or generated, or if the activity ribulokinase is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 63 to 529-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a ribulokinase, or if the activity of the polypeptide B0063, preferably represented by SEQ ID NO. 134300, or a homolog or fragment thereof, or if the activity of a polypeptide encoded by a nucleic acid molecule comprising the nucleic acid SEQ ID NO. 134299, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 134299 or polypeptide SEQ ID NO. 134300, respectively, or a homolog or a fragment thereof, is increased or generated, or if the activity ribulokinase is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 39 to 119-percent is conferred as compared to a corresponding non-transformed wild type non-human organism.

Accordingly, in one embodiment, an increase of citrate in a non-human organism, as compared to a corresponding non-transformed wild type non-human organism, is conferred in the process of the invention, if the activity of a polypeptide showing the activity of a glucose dehydrogenase, or if the activity of the polypeptide B0124, preferably represented by SEQ ID NO. 35483, or a homolog or fragment thereof, or if the activity of a polypeptide encoded by a nucleic acid molecule comprising the nucleic acid SEQ ID NO. 35482, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 35482 or polypeptide SEQ ID NO. 35483, respectively, or a homolog or a fragment thereof, is increased or generated, or if the activity glucose dehydrogenase is increased or generated in a non-human organism, like a microorganism or a plant cell, plant or part thereof, especially with non-targeted localization, whereby the respective line disclose in column 7 the fine chemical citrate. For example, an increase of the citrate of at least 1 percent, particularly in a range of 91 to 837-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a acyl-CoA dehydrogenase, or if the activity of the polypeptide B0221, preferably represented by SEQ ID NO. 35591, or a homolog or fragment thereof, or if the activity of a polypeptide encoded by a nucleic acid molecule comprising the nucleic acid SEQ ID NO. 35590, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 35590 or polypeptide SEQ ID NO. 35591, respectively, or a homolog or a fragment thereof, is increased or generated, or if the activity acyl-CoA dehydrogenase is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 49 to 70-percent is conferred as compared to a corresponding non-transformed wild type non-human organism.

Accordingly, in one embodiment, an increase of citrate in a non-human organism, as compared to a corresponding non-transformed wild type non-human organism, is conferred in the process of the invention, if the activity of a polypeptide showing the activity of a betaine aldehyde dehydrogenase, or if the activity of the polypeptide B0312, preferably represented by SEQ ID NO. 121112, or a homolog or fragment thereof, or if the activity of a polypeptide encoded by a nucleic acid molecule comprising the nucleic acid SEQ ID NO. 121111, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 121111 or polypeptide SEQ ID NO. 121112, respectively, or a homolog or a fragment thereof, is increased or generated, or if the activity betaine aldehyde dehydrogenase is increased or generated in a non-human organism, like a microorganism or a plant cell, plant or part thereof, especially with non-targeted localization, whereby the respective line disclose in column 7 the fine chemical citrate. For example, an increase of the citrate of at least 1 percent, particularly in a range of 126 to 165-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a ATP-binding component of a transport system, or if the activity of the polypeptide B0449, preferably represented by SEQ ID NO. 7334, or a homolog or fragment thereof, or if the activity of a polypeptide encoded by a nucleic acid molecule comprising the nucleic acid SEQ ID NO. 7333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 7333 or polypeptide SEQ ID NO. 7334, respectively, or a homolog or a fragment thereof, is increased or generated, or if the activity ATP-binding component of a transport system is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 94 to 471-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a membrane transport protein, or if the activity of the polypeptide B0486, preferably represented by SEQ ID NO. 7687, or a homolog or fragment thereof, or if the activity of a polypeptide encoded by a nucleic acid molecule comprising the nucleic acid SEQ ID NO. 7686, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 7686 or polypeptide SEQ ID NO. 7687, respectively, or a homolog or a fragment thereof, is increased or generated, or if the activity membrane transport protein is increased or generated in a non-human organism, like a microorganism organism or a plant cell, plant or part thereof, especially with non-targeted localization, whereby the respective line disclose in column 7 the fine chemical malate. For example, an increase of the malate of at least 1 percent, particularly in a range of 61 to 99-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a membrane transport protein, or if the activity of the polypeptide B0486, preferably represented by SEQ ID NO. 7687, or a homolog or fragment thereof, or if the activity of a polypeptide encoded by a nucleic acid molecule comprising the nucleic acid SEQ ID NO. 7686, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 7686 or polypeptide SEQ ID NO. 7687, respectively, or a homolog or a fragment thereof, is increased or generated, or if the activity membrane transport protein is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 31 to 56-percent is conferred as compared to a corresponding non-transformed wild type non-human organism.

Accordingly, in one embodiment, an increase of citrate in a non-human organism, as compared to a corresponding non-transformed wild type non-human organism, is conferred in the process of the invention, if the activity of a polypeptide showing the activity of a glycerate kinase, or if the activity of the polypeptide B0514, preferably represented by SEQ ID NO. 134403, or a homolog or fragment thereof, or if the activity of a polypeptide encoded by a nucleic acid molecule comprising the nucleic acid SEQ ID NO. 134402, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 134402 or polypeptide SEQ ID NO. 134403, respectively, or a homolog or a fragment thereof, is increased or generated, or if the activity glycerate kinase is increased or generated in a non-human organism, like a microorganism or a plant cell, plant or part thereof, especially with non-targeted localization, whereby the respective line disclose in column 7 the fine chemical citrate. For example, an increase of the citrate of at least 1 percent, particularly in a range of 88 to 115-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a isochorismate synthase, or if the activity of the polypeptide B0593, preferably represented by SEQ ID NO. 35968, or a homolog or fragment thereof, or if the activity of a polypeptide encoded by a nucleic acid molecule comprising the nucleic acid SEQ ID NO. 35967, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 35967 or polypeptide SEQ ID NO. 35968, respectively, or a homolog or a fragment thereof, is increased or generated, or if the activity isochorismate synthase is increased or generated in a non-human organism, like a microorganism or a plant cell, plant or part thereof, especially with plastidic localization, whereby the respective line disclose in column 7 the fine chemical fumarate. For example, an increase of the fumarate of at least 1 percent, particularly in a range of 130 to 365-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a isochorismate synthase, or if the activity of the polypeptide B0593, preferably represented by SEQ ID NO. 35968, or a homolog or fragment thereof, or if the activity of a polypeptide encoded by a nucleic acid molecule comprising the nucleic acid SEQ ID NO. 35967, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 35967 or polypeptide SEQ ID NO. 35968, respectively, or a homolog or a fragment thereof, is increased or generated, or if the activity isochorismate synthase is increased or generated in a non-human organism, like a microorganism or a plant cell, plant or part thereof, especially with plastidic localization, whereby the respective line disclose in column 7 the fine chemical malate. For example, an increase of the malate of at least 1 percent, particularly in a range of 168 to 536-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a zinc transporter, or if the activity of the polypeptide B0752, preferably represented by SEQ ID NO. 36115, or a homolog or fragment thereof, or if the activity of a polypeptide encoded by a nucleic acid molecule comprising the nucleic acid SEQ ID NO. 36114, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 36114 or polypeptide SEQ ID NO. 36115, respectively, or a homolog or a fragment thereof, is increased or generated, or if the activity zinc transporter is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 158 to 628-percent is conferred as compared to a corganism responding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a b0801-protein, or if the activity of the polypeptide B0801, preferably represented by SEQ ID NO. 75808, or a homolog or fragment thereof, or if the activity of a polypeptide encoded by a nucleic acid molecule comprising the nucleic acid SEQ ID NO. 75807, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 75807 or polypeptide SEQ ID NO. 75808, respectively, or a homolog or a fragment thereof, is increased or generated, or if the activity b0801-protein is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 67 to 135-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a leucyl/phenylalanyl-tRNA-protein transferase, or if the activity of the polypeptide B0885, preferably represented by SEQ ID NO. 36490, or a homolog or fragment thereof, or if the activity of a polypeptide encoded by a nucleic acid molecule comprising the nucleic acid SEQ ID NO. 36489, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 36489 or polypeptide SEQ ID NO. 36490, respectively, or a homolog or a fragment thereof, is increased or generated, or if the activity leucyl/phenylalanyl-tRNA-protein transferase is increased or generated in a non-human organism, like a microorganism or a plant cell, plant or part thereof, especially with non-targeted localization, whereby the respective line disclose in column 7 the fine chemical malate.

For example, an increase of the malate of at least 1 percent, particularly in a range of 134 to 141-percent is conferred as compared to a corresponding non-transformed wild type non-human organism.

Accordingly, in one embodiment, an increase of vitamin c and/or threonic acid in a non-human organism, as compared to a corresponding non-transformed wild type non-human organism, is conferred in the process of the invention, if the activity of a polypeptide showing the activity of a methylglyoxal synthase, or if the activity of the polypeptide B0963, preferably represented by SEQ ID NO. 36671, or a homolog or fragment thereof, or if the activity of a polypeptide encoded by a nucleic acid molecule comprising the nucleic acid SEQ ID NO. 36670, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 36670 or polypeptide SEQ ID NO. 36671, respectively, or a homolog or a fragment thereof, is increased or generated, or if the activity methylglyoxal synthase is increased or generated in a non-human organism, like a microorganism or a plant cell, plant or part thereof, especially with non-targeted localization, whereby the respective line disclose in column 7 the fine chemical vitamin c and/or threonic acid. For example, an increase of the vitamin c and/or threonic acid of at least 1 percent, particularly in a range of 43 to 81-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a b1003-protein, or if the activity of the polypeptide B1003, preferably represented by SEQ ID NO. 7942, or a homolog or fragment thereof, or if the activity of a polypeptide encoded by a nucleic acid molecule comprising the nucleic acid SEQ ID NO. 7941, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 7941 or polypeptide SEQ ID NO. 7942, respectively, or a homolog or a fragment thereof, is increased or generated, or if the activity b1003-protein is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 107 to 1076-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a membrane protein, or if the activity of the polypeptide B1255, preferably represented by SEQ ID NO. 37484, or a homolog or fragment thereof, or if the activity of a polypeptide encoded by a nucleic acid molecule comprising the nucleic acid SEQ ID NO. 37483, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 37483 or polypeptide SEQ ID NO. 37484, respectively, or a homolog or a fragment thereof, is increased or generated, or if the activity membrane protein is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 50 to 119-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a b1285-protein, or if the activity of the polypeptide B1285, preferably represented by SEQ ID NO. 134656, or a homolog or fragment thereof, or if the activity of a polypeptide encoded by a nucleic acid molecule comprising the nucleic acid SEQ ID NO. 134655, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 134655 or polypeptide SEQ ID NO. 134656, respectively, or a homolog or a fragment thereof, is increased or generated, or if the activity b1285-protein is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 31 to 37-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a phenylacetic acid degradation protein, or if the activity of the polypeptide B1390, preferably represented by SEQ ID NO. 134698, or a hornolog or fragment thereof, or if the activity of a polypeptide encoded by a nucleic acid molecule comprising the nucleic acid SEQ ID NO. 134697, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 134697 or polypeptide SEQ ID NO. 134698, respectively, or a homolog or a fragment thereof, is increased or generated, or if the activity phenylacetic acid degradation protein is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 60 to 182-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a b1425-protein, or if the activity of the polypeptide B1425, preferably represented by SEQ ID NO. 134757, or a homolog or fragment thereof, or if the activity of a polypeptide encoded by a nucleic acid molecule comprising the nucleic acid SEQ ID NO. 134756, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 134756 or polypeptide SEQ ID NO. 134757, respectively, or a homolog or a fragment thereof, is increased or generated, or if the activity b1425-protein is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 61 to 256-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a lipoprotein precursor, or if the activity of the polypeptide B1431, preferably represented by SEQ ID NO. 38267, or a homolog or fragment thereof, or if the activity of a polypeptide encoded by a nucleic acid molecule comprising the nucleic acid SEQ ID NO. 38266, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 38266 or polypeptide SEQ ID NO. 38267, respectively, or a homolog or a fragment thereof, is increased or generated, or if the activity lipoprotein precursor is increased or generated in a non-human organism, like a microorganism or a plant cell, plant or part thereof, especially with plastidic localization, whereby the respective line disclose in column 7 the fine chemical succinate. For example, an increase of the succinate of at least 1 percent, particularly in a range of 29 to 44-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a b1445-protein, or if the activity of the polypeptide B1445, preferably represented by SEQ ID NO. 38290, or a homolog or fragment thereof, or if the activity of a polypeptide encoded by a nucleic acid molecule comprising the nucleic acid SEQ ID NO. 38289, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 38289 or polypeptide SEQ ID NO. 38290, respectively, or a homolog or a fragment thereof, is increased or generated, or if the activity b1445-protein is increased or generated in a non-human organism, like a microorganism or a plant cell, plant or part thereof, especially with non-targeted localization, whereby the respective line disclose in column 7 the fine chemical citramalate. For example, an increase of the citramalate of at least 1 percent, particularly in a range of 67 to 102-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a b1522-protein, or if the activity of the polypeptide B1522, preferably represented by SEQ ID NO. 7948, or a homolog or fragment thereof, or if the activity of a polypeptide encoded by a nucleic acid molecule comprising the nucleic acid SEQ ID NO. 7947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 7947 or polypeptide SEQ ID NO. 7948, respectively, or a homolog or a fragment thereof, is increased or generated, or if the activity b1522-protein is increased or generated in a non-human organism, like a microorganism or a plant cell, plant or part thereof, especially with non-targeted localization, whereby the respective line disclose in column 7 the fine chemical citramalate. For example, an increase of the citramalate of at least 1 percent, particularly in a range of 41 to 92-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a b1522-protein, or if the activity of the polypeptide B1522, preferably represented by SEQ ID NO. 7948, or a homolog or fragment thereof, or if the activity of a polypeptide encoded by a nucleic acid molecule comprising the nucleic acid SEQ ID NO. 7947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 7947 or polypeptide SEQ ID NO. 7948, respectively, or a homolog or a fragment thereof, is increased or generated, or if the activity b1522-protein is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 52 to 311-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a Fe—S subunit of oxidoreductase, or if the activity of the polypeptide B1589, preferably represented by SEQ ID NO. 78155, or a homolog or fragment thereof, or if the activity of a polypeptide encoded by a nucleic acid molecule comprising the nucleic acid SEQ ID NO. 78154, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 78154 or polypeptide SEQ ID NO. 78155, respectively, or a homolog or a fragment thereof, is increased or generated, or if the activity Fe—S subunit of oxidoreductase is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 117 to 167-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a fumarate hydratase, or if the activity of the polypeptide B1611, preferably represented by SEQ ID NO. 115065, or a homolog or fragment thereof, or if the activity of a polypeptide encoded by a nucleic acid molecule comprising the nucleic acid SEQ ID NO. 115064, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 115064 or polypeptide SEQ ID NO. 115065, respectively, or a homolog or a fragment thereof, is increased or generated, or if the activity fumarate hydratase is increased or generated in a non-human organism, like a microorganism or a plant cell, plant or part thereof, especially with plastidic localization, whereby the respective line disclose in column 7 the fine chemical fumarate. For example, an increase of the fumarate of at least 1 percent, particularly in a range of 187 to 514-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a electron transport complex protein, or if the activity of the polypeptide B1627, preferably represented by SEQ ID NO. 38346, or a homolog or fragment thereof, or if the activity of a polypeptide encoded by a nucleic acid molecule comprising the nucleic acid SEQ ID NO. 38345, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 38345 or polypeptide SEQ ID NO. 38346, respectively, or a homolog or a fragment thereof, is increased or generated, or if the activity electron transport complex protein is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 35 to 56-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a methylglyoxalase, or if the activity of the polypeptide B1651, preferably represented by SEQ ID NO. 78266, or a homolog or fragment thereof, or if the activity of a polypeptide encoded by a nucleic acid molecule comprising the nucleic acid SEQ ID NO. 78265, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 78265 or polypeptide SEQ ID NO. 78266, respectively, or a homolog or a fragment thereof, is increased or generated, or if the activity methylglyoxalase is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 27 to 37-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a b1670-protein, or if the activity of the polypeptide B1670, preferably represented by SEQ ID NO. 78754, or a homolog or fragment thereof, or if the activity of a polypeptide encoded by a nucleic acid molecule comprising the nucleic acid SEQ ID NO. 78753, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 78753 or polypeptide SEQ ID NO. 78754, respectively, or a homolog or a fragment thereof, is increased or generated, or if the activity b1670-protein is increased or generated in a non-human organism, like a microorganism or a plant cell, plant or part thereof, especially with non-targeted localization, whereby the respective line disclose in column 7 the fine chemical citramalate. For example, an increase of the citramalate of at least 1 percent, particularly in a range of 49 to 89-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a b1670-protein, or if the activity of the polypeptide B1670, preferably represented by SEQ ID NO. 78754, or a homolog or fragment thereof, or if the activity of a polypeptide encoded by a nucleic acid molecule comprising the nucleic acid SEQ ID NO. 78753, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 78753 or polypeptide SEQ ID NO. 78754, respectively, or a homolog or a fragment thereof, is increased or generated, or if the activity b1670-protein is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 60 to 138-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a b1672-protein, or if the activity of the polypeptide B1672, preferably represented by SEQ ID NO. 78772, or a homolog or fragment thereof, or if the activity of a polypeptide encoded by a nucleic acid molecule comprising the nucleic acid SEQ ID NO. 78771, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 78771 or polypeptide SEQ ID NO. 78772, respectively, or a homolog or a fragment thereof, is increased or generated, or if the activity b1672-protein is increased or generated in a non-human organism, like a microorganism or a plant cell, plant or part thereof, especially with non-targeted localization, whereby the respective line disclose in column 7 the fine chemical citramalate. For example, an increase of the citramalate of at least 1 percent, particularly in a range of 43 to 84-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a b1672-protein, or if the activity of the polypeptide B1672, preferably represented by SEQ ID NO. 78772, or a homolog or fragment thereof, or if the activity of a polypeptide encoded by a nucleic acid molecule comprising the nucleic acid SEQ ID NO. 78771, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 78771 or polypeptide SEQ ID NO. 78772, respectively, or a homolog or a fragment thereof, is increased or generated, or if the activity b1672-protein is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 79 to 152-percent is conferred as compared to a corresponding non-transformed wild type non-human organism.

Accordingly, in one embodiment, an increase of vitamin c and/or threonic acid in a non-human organism, as compared to a corresponding non-transformed wild type non-human organism, is conferred in the process of the invention, if the activity of a polypeptide showing the activity of a b1672-protein, or if the activity of the polypeptide B1672, preferably represented by SEQ ID NO. 78772, or a homolog or fragment thereof, or if the activity of a polypeptide encoded by a nucleic acid molecule comprising the nucleic acid SEQ ID NO. 78771, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 78771 or polypeptide SEQ ID NO. 78772, respectively, or a homolog or a fragment thereof, is increased or generated, or if the activity b1672-protein is increased or generated in a non-human organism, like a microorganism or a plant cell, plant or part thereof, especially with non-targeted localization, whereby the respective line disclose in column 7 the fine chemical vitamin c and/or threonic acid. For example, an increase of the vitamin c and/or threonic acid of at least 1 percent, particularly in a range of 30 to 48-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a b1742-protein, or if the activity of the polypeptide B1742, preferably represented by SEQ ID NO. 134763, or a homolog or fragment thereof, or if the activity of a polypeptide encoded by a nucleic acid molecule comprising the nucleic acid SEQ ID NO. 134762, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 134762 or polypeptide SEQ ID NO. 134763, respectively, or a homolog or a fragment thereof, is increased or generated, or if the activity b1742-protein is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 48 to 86-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a b1742-protein, or if the activity of the polypeptide B1742, preferably represented by SEQ ID NO. 134763, or a homolog or fragment thereof, or if the activity of a polypeptide encoded by a nucleic acid molecule comprising the nucleic acid SEQ ID NO. 134762, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 134762 or polypeptide SEQ ID NO. 134763, respectively, or a homolog or a fragment thereof, is increased or generated, or if the activity b1742-protein is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 33 to 52-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a arginine N-succinyltransferase, or if the activity of the polypeptide B1747, preferably represented by SEQ ID NO. 78780, or a homolog or fragment thereof, or if the activity of a polypeptide encoded by a nucleic acid molecule comprising the nucleic acid SEQ ID NO. 78779, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 78779 or polypeptide SEQ ID NO. 78780, respectively, or a homolog or a fragment thereof, is increased or generated, or if the activity arginine N-succinyltransferase is increased or generated in a non-human organism, like a microorganism or a plant cell, plant or part thereof, especially with plastidic localization, whereby the respective line disclose in column 7 the fine chemical succinate. For example, an increase of the succinate of at least 1 percent, particularly in a range of 51 to 158-percent is conferred as compared to a corresponding non-transformed wild type non-human organism.

Accordingly, in one embodiment, an increase of vitamin c and/or threonic acid in a non-human organism, as compared to a corresponding non-transformed wild type non-human organism, is conferred in the process of the invention, if the activity of a polypeptide showing the activity of a ABC transporter permease protein, or if the activity of the polypeptide B1755, preferably represented by SEQ ID NO. 78853, or a homolog or fragment thereof, or if the activity of a polypeptide encoded by a nucleic acid molecule comprising the nucleic acid SEQ ID NO. 78852, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 78852 or polypeptide SEQ ID NO. 78853, respectively, or a homolog or a fragment thereof, is increased or generated, or if the activity ABC transporter permease protein is increased or generated in a non-human organism, like a microorganism or a plant cell, plant or part thereof, especially with non-targeted localization, whereby the respective line disclose in column 7 the fine chemical vitamin c and/or threonic acid. For example, an increase of the vitamin c and/or threonic acid of at least 1 percent, particularly in a range of 30 to 46-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a protease, or if the activity of the polypeptide B1845, preferably represented by SEQ ID NO. 38574, or a homolog or fragment thereof, or if the activity of a polypeptide encoded by a nucleic acid molecule comprising the nucleic acid SEQ ID NO. 38573, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 38573 or polypeptide SEQ ID NO. 38574, respectively, or a homolog or a fragment thereof, is increased or generated, or if the activity protease is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 92 to 424-percent is conferred as compared to a corresponding non-transformed wild type non-human organism.

Accordingly, in one embodiment, an increase of vitamin c and/or threonic acid in a non-human organism, as compared to a corresponding non-transformed wild type non-human organism, is conferred in the process of the invention, if the activity of a polypeptide showing the activity of a dATP pyrophosphohydrolase, or if the activity of the polypeptide B1865, preferably represented by SEQ ID NO. 134784, or a homolog or fragment thereof, or if the activity of a polypeptide encoded by a nucleic acid molecule comprising the nucleic acid SEQ ID NO. 134783, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 134783 or polypeptide SEQ ID NO. 134784, respectively, or a homolog or a fragment thereof, is increased or generated, or if the activity dATP pyrophosphohydrolase is increased or generated in a non-human organism, like a microorganism or a plant cell, plant or part thereof, especially with non-targeted localization, whereby the respective line disclose in column 7 the fine chemical vitamin c and/or threonic acid. For example, an increase of the vitamin c and/or threonic acid of at least 1 percent, particularly in a range of 38 to 301-percent is conferred as compared to a corganism responding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a b1898-protein, or if the activity of the polypeptide B1898, preferably represented by SEQ ID NO. 38768, or a homolog or fragment thereof, or if the activity of a polypeptide encoded by a nucleic acid molecule comprising the nucleic acid SEQ ID NO. 38767, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 38767 or polypeptide SEQ ID NO. 38768, respectively, or a homolog or a fragment thereof, is increased or generated, or if the activity b1898-protein is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 35 to 94-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a flagellar M-ring protein, or if the activity of the polypeptide B1938, preferably represented by SEQ ID NO. 134865, or a homolog or fragment thereof, or if the activity of a polypeptide encoded by a nucleic acid molecule comprising the nucleic acid SEQ ID NO. 134864, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 134864 or polypeptide SEQ ID NO. 134865, respectively, or a homolog or a fragment thereof, is increased or generated, or if the activity flagellar M-ring protein is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 71 to 324-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a flagellar M-ring protein, or if the activity of the polypeptide B1938, preferably represented by SEQ ID NO. 134865, or a homolog or fragment thereof, or if the activity of a polypeptide encoded by a nucleic acid molecule comprising the nucleic acid SEQ ID NO. 134864, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 134864 or polypeptide SEQ ID NO. 134865, respectively, or a homolog or a fragment thereof, is increased or generated, or if the activity flagellar M-ring protein is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 62 to 332-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a flagellar M-ring protein, or if the activity of the polypeptide B1938, preferably represented by SEQ ID NO. 134865, or a homolog or fragment thereof, or if the activity of a polypeptide encoded by a nucleic acid molecule comprising the nucleic acid SEQ ID NO. 134864, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 134864 or polypeptide SEQ ID NO. 134865, respectively, or a homolog or a fragment thereof, is increased or generated, or if the activity flagellar M-ring protein is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 41 to 171-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a O-antigen chain length determinant, or if the activity of the polypeptide B2027, preferably represented by SEQ ID NO. 79218, or a homolog or fragment thereof, or if the activity of a polypeptide encoded by a nucleic acid molecule comprising the nucleic acid SEQ ID NO. 79217, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 79217 or polypeptide SEQ ID NO. 79218, respectively, or a homolog or a fragment thereof, is increased or generated, or if the activity O-antigen chain length determinant is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 77 to 186 percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a b2399-protein, or if the activity of the polypeptide B2399, preferably represented by SEQ ID NO. 39238, or a homolog or fragment thereof, or if the activity of a polypeptide encoded by a nucleic acid molecule comprising the nucleic acid SEQ ID NO. 39237, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 39237 or polypeptide SEQ ID NO. 39238, respectively, or a homolog or a fragment thereof, is increased or generated, or if the activity b2399-protein is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 190 to 3230-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a b2399-protein, or if the activity of the polypeptide B2399, preferably represented by SEQ ID NO. 39238, or a homolog or fragment thereof, or if the activity of a polypeptide encoded by a nucleic acid molecule comprising the nucleic acid SEQ ID NO. 39237, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 39237 or polypeptide SEQ ID NO. 39238, respectively, or a homolog or a fragment thereof, is increased or generated, or if the activity b2399-protein is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 86 to 421-percent is conferred as compared to a corresponding non-transformed wild type non-human organism.

Accordingly, in one embodiment, an increase of vitamin c and/or threonic acid in a non-human organism, as compared to a corresponding non-transformed wild type non-human organism, is conferred in the process of the invention, if the activity of a polypeptide showing the activity of a b2399-protein, or if the activity of the polypeptide B2399, preferably represented by SEQ ID NO. 39238, or a homolog or fragment thereof, or if the activity of a polypeptide encoded by a nucleic acid molecule comprising the nucleic acid SEQ ID NO. 39237, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 39237 or polypeptide SEQ ID NO. 39238, respectively, or a homolog or a fragment thereof, is increased or generated, or if the activity b2399-protein is increased or generated in a non-human organism, like a microorganism or a plant cell, plant or part thereof, especially with non-targeted localization, whereby the respective line disclose in column 7 the fine chemical vitamin c and/or threonic acid. For example, an increase of the vitamin c and/or threonic acid of at least 1 percent, particularly in a range of 45 to 235-percent is conferred as compared to a corresponding non-transformed wild type non-human organism.

Accordingly, in one embodiment, an increase of vitamin c and/or threonic acid in a non-human organism, as compared to a corresponding non-transformed wild type non-human organism, is conferred in the process of the invention, if the activity of a polypeptide showing the activity of a cysteine synthase A, or if the activity of the polypeptide B2414, preferably represented by SEQ ID NO. 39301, or a homolog or fragment thereof, or if the activity of a polypeptide encoded by a nucleic acid molecule comprising the nucleic acid SEQ ID NO. 39300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 39300 or polypeptide SEQ ID NO. 39301, respectively, or a homolog or a fragment thereof, is increased or generated, or if the activity cysteine synthase A is increased or generated in a non-human organism, like a microorganism or a plant cell, plant or part thereof, especially with non-targeted localization, whereby the respective line disclose in column 7 the fine chemical vitamin c and/or threonic acid. For example, an increase of the vitamin c and/or threonic acid of at least 1 percent, particularly in a range of 30 to 46-percent is conferred as compared to a corresponding non-transformed wild type non-human organism.

Accordingly, in one embodiment, an increase of vitamin c and/or threonic acid in a non-human organism, as compared to a corresponding non-transformed wild type non-human organism, is conferred in the process of the invention, if the activity of a polypeptide showing the activity of a cysteine synthase B, or if the activity of the polypeptide B2421, preferably represented by SEQ ID NO. 134925, or a homolog or fragment thereof, or if the activity of a polypeptide encoded by a nucleic acid molecule comprising the nucleic acid SEQ ID NO. 134924, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 134924 or polypeptide SEQ ID NO. 134925, respectively, or a homolog or a fragment thereof, is increased or generated, or if the activity cysteine synthase B is increased or generated in a non-human organism, like a microorganism or a plant cell, plant or part thereof, especially with plastidic localization, whereby the respective line disclose in column 7 the fine chemical vitamin c and/or threonic acid. For example, an increase of the vitamin c and/or threonic acid of at least 1 percent, particularly in a range of 38 to 197-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a transcriptional regulator, or if the activity of the polypeptide B2634, preferably represented by SEQ ID NO. 40727, or a homolog or fragment thereof, or if the activity of a polypeptide encoded by a nucleic acid molecule comprising the nucleic acid SEQ ID NO. 40726, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 40726 or polypeptide SEQ ID NO. 40727, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 134 to 861-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a transcriptional regulator, or if the activity of the polypeptide B2634, preferably represented by SEQ ID NO. 40727, or a homolog or fragment thereof, or if the activity of a polypeptide encoded by a nucleic acid molecule comprising the nucleic acid SEQ ID NO. 40726, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 40726 or polypeptide SEQ ID NO. 40727, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 139 to 511-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a murein transglycosylase, or if the activity of the polypeptide B2701, preferably represented by SEQ ID NO. 80757, or a homolog or fragment thereof, or if the activity of a polypeptide encoded by a nucleic acid molecule comprising the nucleic acid SEQ ID NO. 80756, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 80756 or polypeptide SEQ ID NO. 80757, respectively, or a homolog or a fragment thereof, is increased or generated, or if the activity murein transglycosylase is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 33 to 60-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a 2-C-methyl-D-erythritol 4-phosphate cytidylyltransferase, or if the activity of the polypeptide B2747, preferably represented by SEQ ID NO. 81076, or a homolog or fragment thereof, or if the activity of a polypeptide encoded by a nucleic acid molecule comprising the nucleic acid SEQ ID NO. 81075, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 81075 or polypeptide SEQ ID NO. 81076, respectively, or a homolog or a fragment thereof, is increased or generated, or if the activity 2-C-methyl-D-erythritol 4-phosphate cytidylyltransferase is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 33 to 53-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a amino-acid acetyltransferase, or if the activity of the polypeptide B2818, preferably represented by SEQ ID NO. 81577, or a homolog or fragment thereof, or if the activity of a polypeptide encoded by a nucleic acid molecule comprising the nucleic acid SEQ ID NO. 81576, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 81576 or polypeptide SEQ ID NO. 81577, respectively, or a homolog or a fragment thereof, is increased or generated, or if the activity amino-acid acetyltransferase is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 30 to 59-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a xanthine dehydrogenase, or if the activity of the polypeptide B2866, preferably represented by SEQ ID NO. 115865, or a homolog or fragment thereof, or if the activity of a polypeptide encoded by a nucleic acid molecule comprising the nucleic acid SEQ ID NO. 115864, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 115864 or polypeptide SEQ ID NO. 115865, respectively, or a homolog or a fragment thereof, is increased or generated, or if the activity xanthine dehydrogenase is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 26 to 34-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a cystathionine beta-lyase, or if the activity of the polypeptide B3008, preferably represented by SEQ ID NO. 135894, or a homolog or fragment thereof, or if the activity of a polypeptide encoded by a nucleic acid molecule comprising the nucleic acid SEQ ID NO. 135893, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 135893 or polypeptide SEQ ID NO. 135894, respectively, or a homolog or a fragment thereof, is increased or generated, or if the activity cystathionine beta-lyase is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 26 to 63-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a methyltransferase, or if the activity of the polypeptide B3262, preferably represented by SEQ ID NO. 42503, or a homolog or fragment thereof, or if the activity of a polypeptide encoded by a nucleic acid molecule comprising the nucleic acid SEQ ID NO. 42502, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 42502 or polypeptide SEQ ID NO. 42503, respectively, or a homolog or a fragment thereof, is increased or generated, or if the activity methyltransferase is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 69 to 208-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a methyltransferase, or if the activity of the polypeptide B3262, preferably represented by SEQ ID NO. 42503, or a homolog or fragment thereof, or if the activity of a polypeptide encoded by a nucleic acid molecule comprising the nucleic acid SEQ ID NO. 42502, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 42502 or polypeptide SEQ ID NO. 42503, respectively, or a homolog or a fragment thereof, is increased or generated, or if the activity methyltransferase is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 76 to 135-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a lipopolysaccharide core biosynthesis protein, or if the activity of the polypeptide B3624, preferably represented by SEQ ID NO. 136078, or a homolog or fragment thereof, or if the activity of a polypeptide encoded by a nucleic acid molecule comprising the nucleic acid SEQ ID NO. 136077, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 136077 or polypeptide SEQ ID NO. 136078, respectively, or a homolog or a fragment thereof, is increased or generated, or if the activity lipopolysaccharide core biosynthesis protein is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 69 to 1749-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a b3777-protein, or if the activity of the polypeptide B3777, preferably represented by SEQ ID NO. 136099, or a homolog or fragment thereof, or if the activity of a polypeptide encoded by a nucleic acid molecule comprising the nucleic acid SEQ ID NO. 136098, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 136098 or polypeptide SEQ ID NO. 136099, respectively, or a homolog or a fragment thereof, is increased or generated, or if the activity b3777-protein is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 29 to 54-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a b3791-protein, or if the activity of the polypeptide B3791, preferably represented by SEQ ID NO. 83727, or a homolog or fragment thereof, or if the activity of a polypeptide encoded by a nucleic acid molecule comprising the nucleic acid SEQ ID NO. 83726, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 83726 or polypeptide SEQ ID NO. 83727, respectively, or a homolog or a fragment thereof, is increased or generated, or if the activity b3791-protein is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 52 to 264-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a b3791-protein, or if the activity of the polypeptide B3791, preferably represented by SEQ ID NO. 83727, or a homolog or fragment thereof, or if the activity of a polypeptide encoded by a nucleic acid molecule comprising the nucleic acid SEQ ID NO. 83726, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 83726 or polypeptide SEQ ID NO. 83727, respectively, or a homolog or a fragment thereof, is increased or generated, or if the activity b3791-protein is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 29 to 160-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a b3814-protein, or if the activity of the polypeptide B3814, preferably represented by SEQ ID NO. 44197, or a homolog or fragment thereof, or if the activity of a polypeptide encoded by a nucleic acid molecule comprising the nucleic acid SEQ ID NO. 44196, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 44196 or polypeptide SEQ ID NO. 44197, respectively, or a homolog or a fragment thereof, is increased or generated, or if the activity b3814-protein is increased or generated in a non-human organism, like a microorganism or a plant cell, plant or part thereof, especially with non-targeted localization, whereby the respective line disclose in column 7 the fine chemical citramalate. For example, an increase of the citramalate of at least 1 percent, particularly in a range of 41 to 45-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a b3817-protein, or if the activity of the polypeptide B3817, preferably represented by SEQ ID NO. 10709, or a homolog or fragment thereof, or if the activity of a polypeptide encoded by a nucleic acid molecule comprising the nucleic acid SEQ ID NO. 10708, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 10708 or polypeptide SEQ ID NO. 10709, respectively, or a homolog or a fragment thereof, is increased or generated, or if the activity b3817-protein is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 122 to 198-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a threonine efflux protein, or if the activity of the polypeptide B3823, preferably represented by SEQ ID NO. 10727, or a homolog or fragment thereof, or if the activity of a polypeptide encoded by a nucleic acid molecule comprising the nucleic acid SEQ ID NO. 10726, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 10726 or polypeptide SEQ ID NO. 10727, respectively, or a homolog or a fragment thereof, is increased or generated, or if the activity threonine efflux protein is increased or generated in a non-human organism, like a microorganism or a plant cell, plant or part thereof, especially with non-targeted localization, whereby the respective line disclose in column 7 the fine chemical citramalate. For example, an increase of the citramalate of at least 1 percent, particularly in a range of 44 to 151-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a threonine efflux protein, or if the activity of the polypeptide B3823, preferably represented by SEQ ID NO. 10727, or a homolog or fragment thereof, or if the activity of a polypeptide encoded by a nucleic acid molecule comprising the nucleic acid SEQ ID NO. 10726, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 10726 or polypeptide SEQ ID NO. 10727, respectively, or a homolog or a fragment thereof, is increased or generated, or if the activity threonine efflux protein is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 56 to 84-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a b3989-protein, or if the activity of the polypeptide B3989, preferably represented by SEQ ID NO. 44373, or a homolog or fragment thereof, or if the activity of a polypeptide encoded by a nucleic acid molecule comprising the nucleic acid SEQ ID NO. 44372, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 44372 or polypeptide SEQ ID NO. 44373, respectively, or a homolog or a fragment thereof, is increased or generated, or if the activity b3989-protein is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 53 to 90-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a acetyltransferase, or if the activity of the polypeptide B4012, preferably represented by SEQ ID NO. 44379, or a homolog or fragment thereof, or if the activity of a polypeptide encoded by a nucleic acid molecule comprising the nucleic acid SEQ ID NO. 44378, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 44378 or polypeptide SEQ ID NO. 44379, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 58 to 204-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a b4029-protein, or if the activity of the polypeptide B4029, preferably represented by SEQ ID NO. 10741, or a homolog or fragment thereof, or if the activity of a polypeptide encoded by a nucleic acid molecule comprising the nucleic acid SEQ ID NO. 10740, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 10740 or polypeptide SEQ ID NO. 10741, respectively, or a homolog or a fragment thereof, is increased or generated, or if the activity b4029-protein is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 76 to 467-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a b4029-protein, or if the activity of the polypeptide B4029, preferably represented by SEQ ID NO. 10741, or a homolog or fragment thereof, or if the activity of a polypeptide encoded by a nucleic acid molecule comprising the nucleic acid SEQ ID NO. 10740, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 10740 or polypeptide SEQ ID NO. 10741, respectively, or a homolog or a fragment thereof, is increased or generated, or if the activity b4029-protein is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 81 to 511-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a b4029-protein, or if the activity of the polypeptide B4029, preferably represented by SEQ ID NO. 10741, or a homolog or fragment thereof, or if the activity of a polypeptide encoded by a nucleic acid molecule comprising the nucleic acid SEQ ID NO. 10740, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 10740 or polypeptide SEQ ID NO. 10741, respectively, or a homolog or a fragment thereof, is increased or generated, or if the activity b4029-protein is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 29 to 444-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a b4050-protein, or if the activity of the polypeptide B4050, preferably represented by SEQ ID NO. 106533, or a homolog or fragment thereof, or if the activity of a polypeptide encoded by a nucleic acid molecule comprising the nucleic acid SEQ ID NO. 106532, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 106532 or polypeptide SEQ ID NO. 106533, respectively, or a homolog or a fragment thereof, is increased or generated, or if the activity b4050-protein is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 127 to 1497-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a b4050-protein, or if the activity of the polypeptide B4050, preferably represented by SEQ ID NO. 106533, or a homolog or fragment thereof, or if the activity of a polypeptide encoded by a nucleic acid molecule comprising the nucleic acid SEQ ID NO. 106532, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 106532 or polypeptide SEQ ID NO. 106533, respectively, or a homolog or a fragment thereof, is increased or generated, or if the activity b4050-protein is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 52 to 553-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a b4056-protein, or if the activity of the polypeptide B4056, preferably represented by SEQ ID NO. 94543, or a homolog or fragment thereof, or if the activity of a polypeptide encoded by a nucleic acid molecule comprising the nucleic acid SEQ ID NO. 94542, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 94542 or polypeptide SEQ ID NO. 94543, respectively, or a homolog or a fragment thereof, is increased or generated, or if the activity b4056-protein is increased or generated in a non-human organism, like a microorganism or a plant cell, plant or part thereof, especially with non-targeted localization, whereby the respective line disclose in column 7 the fine chemical citramalate. For example, an increase of the citramalate of at least 1 percent, particularly in a range of 41 to 94-percent is conferred as compared to a corresponding non-transformed wild type non-human organism.

Accordingly, in one embodiment, an increase of citrate in a non-human organism, as compared to a corresponding non-transformed wild type non-human organism, is conferred in the process of the invention, if the activity of a polypeptide showing the activity of a aspartase, or if the activity of the polypeptide B4139, preferably represented by SEQ ID NO. 45023, or a homolog or fragment thereof, or if the activity of a polypeptide encoded by a nucleic acid molecule comprising the nucleic acid SEQ ID NO. 45022, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 45022 or polypeptide SEQ ID NO. 45023, respectively, or a homolog or a fragment thereof, is increased or generated, or if the activity aspartase is increased or generated in a non-human organism, like a microorganism or a plant cell, plant or part thereof, especially with non-targeted localization, whereby the respective line disclose in column 7 the fine chemical citrate. For example, an increase of the citrate of at least 1 percent, particularly in a range of 119 to 342-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a acetyltransferase, or if the activity of the polypeptide B4256, preferably represented by SEQ ID NO. 45322, or a homolog or fragment thereof, or if the activity of a polypeptide encoded by a nucleic acid molecule comprising the nucleic acid SEQ ID NO. 45321, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 45321 or polypeptide SEQ ID NO. 45322, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 40 to 45-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a acetyltransferase, or if the activity of the polypeptide B4256, preferably represented by SEQ ID NO. 45322, or a homolog or fragment thereof, or if the activity of a polypeptide encoded by a nucleic acid molecule comprising the nucleic acid SEQ ID NO. 45321, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 45321 or polypeptide SEQ ID NO. 45322, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 201 to 278-percent is conferred as compared to a corresponding non-transformed wild type non-human organism.

Accordingly, in one embodiment, an increase of vitamin c and/or threonic acid in a non-human organism, as compared to a corresponding non-transformed wild type non-human organism, is conferred in the process of the invention, if the activity of a polypeptide showing the activity of a purine-nucleoside phosphorylase, or if the activity of the polypeptide B4384, preferably represented by SEQ ID NO. 45557, or a homolog or fragment thereof, or if the activity of a polypeptide encoded by a nucleic acid molecule comprising the nucleic acid SEQ ID NO. 45556, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 45556 or polypeptide SEQ ID NO. 45557, respectively, or a homolog or a fragment thereof, is increased or generated, or if the activity purine-nucleoside phosphorylase is increased or generated in a non-human organism, like a microorganism or a plant cell, plant or part thereof, especially with non-targeted localization, whereby the respective line disclose in column 7 the fine chemical vitamin c and/or threonic acid. For example, an increase of the vitamin c and/or threonic acid of at least 1 percent, particularly in a range of 36 to 222-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a lysine decarboxylase, or if the activity of the polypeptide D90900, preferably represented by SEQ ID NO. 84080, or a homolog or fragment thereof, or if the activity of a polypeptide encoded by a nucleic acid molecule comprising the nucleic acid SEQ ID NO. 84079, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 84079 or polypeptide SEQ ID NO. 84080, respectively, or a homolog or a fragment thereof, is increased or generated, or if the activity lysine decarboxylase is increased or generated in a non-human organism, like a microorganism organism or a plant cell, plant or part thereof, especially with plastidic localization, whereby the respective line disclose in column 7 the fine chemical succinate. For example, an increase of the succinate of at least 1 percent, particularly in a range of 33 to 49-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a glutaredoxin, or if the activity of the polypeptide GM02LC12622, preferably represented by SEQ ID NO. 10812, or a homolog or fragment thereof, or if the activity of a polypeptide encoded by a nucleic acid molecule comprising the nucleic acid SEQ ID NO. 10811, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 10811 or polypeptide SEQ ID NO. 10812, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 32 to 137-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a GM02LC17485-protein, or if the activity of the polypeptide GM02LC17485, preferably represented by SEQ ID NO. 46406, or a homolog or fragment thereof, or if the activity of a polypeptide encoded by a nucleic acid molecule comprising the nucleic acid SEQ ID NO. 46405, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 46405 or polypeptide SEQ ID NO. 46406, respectively, or a homolog or a fragment thereof, is increased or generated, or if the activity GM02LC17485-protein is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 67 to 224-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a transcription factor, or if the activity of the polypeptide GM02LC21368, preferably represented by SEQ ID NO. 84199, or a homolog or fragment thereof, or if the activity of a polypeptide encoded by a nucleic acid molecule comprising the nucleic acid SEQ ID NO. 84198, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 84198 or polypeptide SEQ ID NO. 84199, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical citramalate. For example, an increase of the citramalate of at least 1 percent, particularly in a range of 42 to 177-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a transcription factor, or if the activity of the polypeptide GM02LC21368, preferably represented by SEQ ID NO. 84199, or a homolog or fragment thereof, or if the activity of a polypeptide encoded by a nucleic acid molecule comprising the nucleic acid SEQ ID NO. 84198, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 84198 or polypeptide SEQ ID NO. 84199, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 71 to 316-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a transcription factor, or if the activity of the polypeptide GM02LC21368, preferably represented by SEQ ID NO. 84199, or a homolog or fragment thereof, or if the activity of a polypeptide encoded by a nucleic acid molecule comprising the nucleic acid SEQ ID NO. 84198, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 84198 or polypeptide SEQ ID NO. 84199, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 102 to 632-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a GM02LC5744-protein, or if the activity of the polypeptide GM02LC5744, preferably represented by SEQ ID NO. 47077, or a homolog or fragment thereof, or if the activity of a polypeptide encoded by a nucleic acid molecule comprising the nucleic acid SEQ ID NO. 47076, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 47076 or polypeptide SEQ ID NO. 47077, respectively, or a homolog or a fragment thereof, is increased or generated, or if the activity GM02LC5744-protein is increased or generated in a non-human organism, like a microorganism or a plant cell, plant or part thereof, especially with non-targeted localization, whereby the respective line disclose in column 7 the fine chemical citramalate. For example, an increase of the citramalate of at least 1 percent, particularly in a range of 47 to 69-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a GM02LC5744-protein, or if the activity of the polypeptide GM02LC5744, preferably represented by SEQ ID NO. 47077, or a homolog or fragment thereof, or if the activity of a polypeptide encoded by a nucleic acid molecule comprising the nucleic acid SEQ ID NO. 47076, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 47076 or polypeptide SEQ ID NO. 47077, respectively, or a homolog or a fragment thereof, is increased or generated, or if the activity GM02LC5744-protein is increased or generated in a non-human organism, like a microorganism organism or a plant cell, plant or part thereof, especially with non-targeted localization, whereby the respective line disclose in column 7 the fine chemical fumarate. For example, an increase of the fumarate of at least 1 percent, particularly in a range of 49 to 104-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a heat shock protein, or if the activity of the polypeptide Sll0170, preferably represented by SEQ ID NO. 48139, or a homolog or fragment thereof, or if the activity of a polypeptide encoded by a nucleic acid molecule comprising the nucleic acid SEQ ID NO. 48138, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 48138 or polypeptide SEQ ID NO. 48139, respectively, or a homolog or a fragment thereof, is increased or generated, or if the activity heat shock protein is increased or generated in a non-human organism, like a microorganism or a plant cell, plant or part thereof, especially with non-targeted localization, whereby the respective line disclose in column 7 the fine chemical citramalate. For example, an increase of the citramalate of at least 1 percent, particularly in a range of 49 to 116-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a agmatinase, or if the activity of the polypeptide Sll0228, preferably represented by SEQ ID NO. 84266, or a homolog or fragment thereof, or if the activity of a polypeptide encoded by a nucleic acid molecule comprising the nucleic acid SEQ ID NO. 84265, preferably the coding region thereof, or a homolog or fragment thereof, e.g. derived from Synechocystis sp., is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 84265 or polypeptide SEQ ID NO. 84266, respectively, or a homolog or a fragment thereof, is increased or generated, or if the activity agmatinase is increased or generated in a non-human organism, like a microorganism or a plant cell, plant or part thereof, especially with plastidic localization, whereby the respective line disclose in column 7 the fine chemical fumarate. For example, an increase of the fumarate of at least 1 percent, particularly in a range of 67 to 78-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a ABC transporter ATP binding component, or if the activity of the polypeptide Sll0240, preferably represented by SEQ ID NO. 84442, or a homolog or fragment thereof, or if the activity of a polypeptide encoded by a nucleic acid molecule comprising the nucleic acid SEQ ID NO. 84441, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 84441 or polypeptide SEQ ID NO. 84442, respectively, or a homolog or a fragment thereof, is increased or generated, or if the activity ABC transporter ATP binding component is increased or generated in a non-human organism, like a microorganism or a plant cell, plant or part thereof, especially with plastidic localization, whereby the respective line disclose in column 7 the fine chemical malate. For example, an increase of the malate of at least 1 percent, particularly in a range of 73 to 176-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a ABC transporter ATP binding component, or if the activity of the polypeptide Sll0240, preferably represented by SEQ ID NO. 84442, or a homolog or fragment thereof, or if the activity of a polypeptide encoded by a nucleic acid molecule comprising the nucleic acid SEQ ID NO. 84441, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 84441 or polypeptide SEQ ID NO. 84442, respectively, or a homolog or a fragment thereof, is increased or generated, or if the activity ABC transporter ATP binding component is increased or generated in a non-human organism, like a microorganism or a plant cell, plant or part thereof, especially with plastidic localization, whereby the respective line disclose in column 7 the fine chemical succinate. For example, an increase of the succinate of at least 1 percent, particularly in a range of 26 to 91-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a oxireductase, or if the activity of the polypeptide Sll0816, preferably represented by SEQ ID NO. 51199, or a homolog or fragment thereof, or if the activity of a polypeptide encoded by a nucleic acid molecule comprising the nucleic acid SEQ ID NO. 51198, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 51198 or polypeptide SEQ ID NO. 51199, respectively, or a homolog or a fragment thereof, is increased or generated, or if the activity oxireductase is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 64 to 228-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a phosphatase, or if the activity of the polypeptide Sll0895, preferably represented by SEQ ID NO. 127110, or a homolog or fragment thereof, or if the activity of a polypeptide encoded by a nucleic acid molecule comprising the nucleic acid SEQ ID NO. 127109, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 127109 or polypeptide SEQ ID NO. 127110, respectively, or a homolog or a fragment thereof, is increased or generated, or if the activity phosphatase is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 34 to 83-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a CDP-diacylglycerol-glycerol-3-phosphate 3-phosphatidyltransferase, or if the activity of the polypeptide Sll1522, preferably represented by SEQ ID NO. 54453, or a homolog or fragment thereof, or if the activity of a polypeptide encoded by a nucleic acid molecule comprising the nucleic acid SEQ ID NO. 54452, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 54452 or polypeptide SEQ ID NO. 54453, respectively, or a homolog or a fragment thereof, is increased or generated, or if the activity CDP-diacylglycerol-glycerol-3-phosphate 3-phosphatidyltransferase is increased or generated in a non-human organism, like a microorganism or a plant cell, plant or part thereof, especially with plastidic localization, whereby the respective line disclose in column 7 the fine chemical citramalate. For example, an increase of the citramalate of at least 1 percent, particularly in a range of 43 to 57-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a CDP-diacylglycerol-glycerol-3-phosphate 3-phosphatidyltransferase, or if the activity of the polypeptide Sll1522, preferably represented by SEQ ID NO. 54453, or a homolog or fragment thereof, or if the activity of a polypeptide encoded by a nucleic acid molecule comprising the nucleic acid SEQ ID NO. 54452, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 54452 or polypeptide SEQ ID NO. 54453, respectively, or a homolog or a fragment thereof, is increased or generated, or if the activity CDP-diacylglycerol-glycerol-3-phosphate 3-phosphatidyltransferase is increased or generated in a non-human organism, like a microorganism or a plant cell, plant or part thereof, especially with plastidic localization, whereby the respective line disclose in column 7 the fine chemical malate. For example, an increase of the malate of at least 1 percent, particularly in a range of 55 to 206-percent is conferred as compared to a corganism responding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a adenylate kinase, or if the activity of the polypeptide Sll1815, preferably represented by SEQ ID NO. 85809, or a homolog or fragment thereof, or if the activity of a polypeptide encoded by a nucleic acid molecule comprising the nucleic acid SEQ ID NO. 85808, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 85808 or polypeptide SEQ ID NO. 85809, respectively, or a homolog or a fragment thereof, is increased or generated, or if the activity adenylate kinase is increased or generated in a non-human organism, like a microorganism or a plant cell, plant or part thereof, especially with mitochondrial localization, whereby the respective line disclose in column 7 the fine chemical succinate. For example, an increase of the succinate of at least 1 percent, particularly in a range of 35 to 414-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a cation-transporting ATPase, or if the activity of the polypeptide Sll1920, preferably represented by SEQ ID NO. 55386, or a homolog or fragment thereof, or if the activity of a polypeptide encoded by a nucleic acid molecule comprising the nucleic acid SEQ ID NO. 55385, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 55385 or polypeptide SEQ ID NO. 55386, respectively, or a homolog or a fragment thereof, is increased or generated, or if the activity cation-transporting ATPase is increased or generated in a non-human organism, like a microorganism or a plant cell, plant or part thereof, especially with non-targeted localization, whereby the respective line disclose in column 7 the fine chemical citramalate. For example, an increase of the citramalate of at least 1 percent, particularly in a range of 42 to 70-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a cation-transporting ATPase, or if the activity of the polypeptide Sll1920, preferably represented by SEQ ID NO. 55386, or a homolog or fragment thereof, or if the activity of a polypeptide encoded by a nucleic acid molecule comprising the nucleic acid SEQ ID NO. 55385, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 55385 or polypeptide SEQ ID NO. 55386, respectively, or a homolog or a fragment thereof, is increased or generated, or if the activity cation-transporting ATPase is increased or generated in a non-human organism, like a microorganism or a plant cell, plant or part thereof, especially with mitochondrial localization, whereby the respective line disclose in column 7 the fine chemical malate. For example, an increase of the malate of at least 1 percent, particularly in a range of 63 to 120-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a fumarate hydratase, or if the activity of the polypeptide Slr0018, preferably represented by SEQ ID NO. 136534, or a homolog or fragment thereof, or if the activity of a polypeptide encoded by a nucleic acid molecule comprising the nucleic acid SEQ ID NO. 136533, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 136533 or polypeptide SEQ ID NO. 136534, respectively, or a homolog or a fragment thereof, is increased or generated, or if the activity fumarate hydratase is increased or generated in a non-human organism, like a microorganism or a plant cell, plant or part thereof, especially with plastidic localization, whereby the respective line disclose in column 7 the fine chemical fumarate. For example, an increase of the fumarate of at least 1 percent, particularly in a range of 167 to 367-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a oxidoreductase, or if the activity of the polypeptide Slr0338, preferably represented by SEQ ID NO. 11991, or a homolog or fragment thereof, or if the activity of a polypeptide encoded by a nucleic acid molecule comprising the nucleic acid SEQ ID NO. 11990, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 11990 or polypeptide SEQ ID NO. 11991, respectively, or a homolog or a fragment thereof, is increased or generated, or if the activity oxidoreductase is increased or generated in a non-human organism, like a microorganism or a plant cell, plant or part thereof, especially with plastidic localization, whereby the respective line disclose in column 7 the fine chemical citramalate. For example, an increase of the citramalate of at least 1 percent, particularly in a range of 58 to 138-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a bifunctional purine biosynthesis protein, or if the activity of the polypeptide Slr0597, preferably represented by SEQ ID NO. 56154, or a homolog or fragment thereof, or if the activity of a polypeptide encoded by a nucleic acid molecule comprising the nucleic acid SEQ ID NO. 56153, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 56153 or polypeptide SEQ ID NO. 56154, respectively, or a homolog or a fragment thereof, is increased or generated, or if the activity bifunctional purine biosynthesis protein is increased or generated in a non-human organism, like a microorganism or a plant cell, plant or part thereof, especially with plastidic localization, whereby the respective line disclose in column 7 the fine chemical fumarate. For example, an increase of the fumarate of at least 1 percent, particularly in a range of 65 to 115-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a malate dehydrogenase, or if the activity of the polypeptide Slr0721, preferably represented by SEQ ID NO. 87398, or a homolog or fragment thereof, or if the activity of a polypeptide encoded by a nucleic acid molecule comprising the nucleic acid SEQ ID NO. 87397, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 87397 or polypeptide SEQ ID NO. 87398, respectively, or a homolog or a fragment thereof, is increased or generated, or if the activity malate dehydrogenase is increased or generated in a non-human organism, like a microorganism or a plant cell, plant or part thereof, especially with plastidic localization, whereby the respective line disclose in column 7 the fine chemical citramalate. For example, an increase of the citramalate of at least 1 percent, particularly in a range of 55 to 692-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a malate dehydrogenase, or if the activity of the polypeptide Slr0721, preferably represented by SEQ ID NO. 87398, or a homolog or fragment thereof, or if the activity of a polypeptide encoded by a nucleic acid molecule comprising the nucleic acid SEQ ID NO. 87397, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 87397 or polypeptide SEQ ID NO. 87398, respectively, or a homolog or a fragment thereof, is increased or generated, or if the activity malate dehydrogenase is increased or generated in a non-human organism, like a microorganism or a plant cell, plant or part thereof, especially with plastidic localization, whereby the respective line disclose in column 7 the fine chemical succinate. For example, an increase of the succinate of at least 1 percent, particularly in a range of 63 to 291-percent is conferred as compared to a corresponding non-transformed wild type non-human organism.

Accordingly, in one embodiment, an increase of glycerate in a non-human organism, as compared to a corresponding non-transformed wild type non-human organism, is conferred in the process of the invention, if the activity of a polypeptide showing the activity of a dihydrolipoamide dehydrogenase, or if the activity of the polypeptide Slr1096, preferably represented by SEQ ID NO. 57735, or a homolog or fragment thereof, or if the activity of a polypeptide encoded by a nucleic acid molecule comprising the nucleic acid SEQ ID NO. 57734, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 57734 or polypeptide SEQ ID NO. 57735, respectively, or a homolog or a fragment thereof, is increased or generated, or if the activity dihydrolipoamide dehydrogenase is increased or generated in a non-human organism, like a microorganism or a plant cell, plant or part thereof, especially with non-targeted localization, whereby the respective line disclose in column 7 the fine chemical glycerate. For example, an increase of the glycerate of at least 1 percent, particularly in a range of 31 to 42-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a dihydrolipoamide dehydrogenase, or if the activity of the polypeptide Slr1096, preferably represented by SEQ ID NO. 57735, or a homolog or fragment thereof, or if the activity of a polypeptide encoded by a nucleic acid molecule comprising the nucleic acid SEQ ID NO. 57734, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 57734 or polypeptide SEQ ID NO. 57735, respectively, or a homolog or a fragment thereof, is increased or generated, or if the activity dihydrolipoamide dehydrogenase is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 24 to 59-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a slr1170-protein, or if the activity of the polypeptide Slr1170, preferably represented by SEQ ID NO. 136870, or a homolog or fragment thereof, or if the activity of a polypeptide encoded by a nucleic acid molecule comprising the nucleic acid SEQ ID NO. 136869, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 136869 or polypeptide SEQ ID NO. 136870, respectively, or a homolog or a fragment thereof, is increased or generated, or if the activity slr1170-protein is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 39 to 81-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a phosphate import ATP-binding protein, or if the activity of the polypeptide Slr1250, preferably represented by SEQ ID NO. 116461, or a homolog or fragment thereof, or if the activity of a polypeptide encoded by a nucleic acid molecule comprising the nucleic acid SEQ ID NO. 116460, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 116460 or polypeptide SEQ ID NO. 116461, respectively, or a homolog or a fragment thereof, is increased or generated, or if the activity phosphate import ATP-binding protein is increased or generated in a non-human organism, like a microorganism or a plant cell, plant or part thereof, especially with mitochondrial localization, whereby the respective line disclose in column 7 the fine chemical fumarate. For example, an increase of the fumarate of at least 1 percent, particularly in a range of 53 to 59-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a gamma-glutamyltranspeptidase, or if the activity of the polypeptide Slr1269, preferably represented by SEQ ID NO. 58059, or a homolog or fragment thereof, or if the activity of a polypeptide encoded by a nucleic acid molecule comprising the nucleic acid SEQ ID NO. 58058, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 58058 or polypeptide SEQ ID NO. 58059, respectively, or a homolog or a fragment thereof, is increased or generated, or if the activity gamma-glutamyltranspeptidase is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 32 to 75-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a photosystem II protein, or if the activity of the polypeptide Slr1739, preferably represented by SEQ ID NO. 58732, or a homolog or fragment thereof, or if the activity of a polypeptide encoded by a nucleic acid molecule comprising the nucleic acid SEQ ID NO. 58731, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 58731 or polypeptide SEQ ID NO. 58732, respectively, or a homolog or a fragment thereof, is increased or generated, or if the activity photosystem II protein is increased or generated in a non-human organism, like a microorganism or a plant cell, plant or part thereof, especially with plastidic localization, whereby the respective line disclose in column 7 the fine chemical malate. For example, an increase of the malate of at least 1 percent, particularly in a range of 73 to 318-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a malonyl CoA-acyl carrier protein transacylase, or if the activity of the polypeptide Slr2023, preferably represented by SEQ ID NO. 59166, or a homolog or fragment thereof, or if the activity of a polypeptide encoded by a nucleic acid molecule comprising the nucleic acid SEQ ID NO. 59165, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 59165 or polypeptide SEQ ID NO. 59166, respectively, or a homolog or a fragment thereof, is increased or generated, or if the activity malonyl CoA-acyl carrier protein transacylase is increased or generated in a non-human organism, like a microorganism or a plant cell, plant or part thereof, especially with plastidic localization, whereby the respective line disclose in column 7 the fine chemical fumarate. For example, an increase of the fumarate of at least 1 percent, particularly in a range of 108 to 384-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a malonyl CoA-acyl carrier protein transacylase, or if the activity of the polypeptide Slr2023, preferably represented by SEQ ID NO. 59166, or a homolog or fragment thereof, or if the activity of a polypeptide encoded by a nucleic acid molecule comprising the nucleic acid SEQ ID NO. 59165, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 59165 or polypeptide SEQ ID NO. 59166, respectively, or a homolog or a fragment thereof, is increased or generated, or if the activity malonyl CoA-acyl carrier protein transacylase is increased or generated in a non-human organism, like a microorganism or a plant cell, plant or part thereof, especially with plastidic localization, whereby the respective line disclose in column 7 the fine chemical succinate. For example, an increase of the succinate of at least 1 percent, particularly in a range of 38 to 152-percent is conferred as compared to a corresponding non-transformed wild type non-human organism.

Accordingly, in one embodiment, an increase of glycerate in a non-human organism, as compared to a corresponding non-transformed wild type non-human organism, is conferred in the process of the invention, if the activity of a polypeptide showing the activity of a photosystem I reaction centre subunit, or if the activity of the polypeptide Smr0004, preferably represented by SEQ ID NO. 136908, or a homolog or fragment thereof, or if the activity of a polypeptide encoded by a nucleic acid molecule comprising the nucleic acid SEQ ID NO. 136907, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 136907 or polypeptide SEQ ID NO. 136908, respectively, or a homolog or a fragment thereof, is increased or generated, or if the activity photosystem I reaction centre subunit is increased or generated in a non-human organism, like a microorganism or a plant cell, plant or part thereof, especially with plastidic localization, whereby the respective line disclose in column 7 the fine chemical glycerate. For example, an increase of the glycerate of at least 1 percent, particularly in a range of 29 to 32-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a Sec-independent protein translocase subunit, or if the activity of the polypeptide TTC0019, preferably represented by SEQ ID NO. 12699, or a homolog or fragment thereof, or if the activity of a polypeptide encoded by a nucleic acid molecule comprising the nucleic acid SEQ ID NO. 12698, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 12698 or polypeptide SEQ ID NO. 12699, respectively, or a homolog or a fragment thereof, is increased or generated, or if the activity Sec-independent protein translocase subunit is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 181 to 395-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a Sec-independent protein translocase subunit, or if the activity of the polypeptide TTC0019, preferably represented by SEQ ID NO. 12699, or a homolog or fragment thereof, or if the activity of a polypeptide encoded by a nucleic acid molecule comprising the nucleic acid SEQ ID NO. 12698, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 12698 or polypeptide SEQ ID NO. 12699, respectively, or a homolog or a fragment thereof, is increased or generated, or if the activity Sec-independent protein translocase subunit is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 31 to 106-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a 2-isopropylmalate synthase, or if the activity of the polypeptide TTC0849, preferably represented by SEQ ID NO. 107557, or a homolog or fragment thereof, or if the activity of a polypeptide encoded by a nucleic acid molecule comprising the nucleic acid SEQ ID NO. 107556, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 107556 or polypeptide SEQ ID NO. 107557, respectively, or a homolog or a fragment thereof, is increased or generated, or if the activity 2-isopropylmalate synthase is increased or generated in a non-human organism, like a microorganism or a plant cell, plant or part thereof, especially with non-targeted localization, whereby the respective line disclose in column 7 the fine chemical citramalate. For example, an increase of the citramalate of at least 1 percent, particularly in a range of 40 to 352-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a multiple antibiotic resistance protein, or if the activity of the polypeptide TTC1193, preferably represented by SEQ ID NO. 61724, or a homolog or fragment thereof, or if the activity of a polypeptide encoded by a nucleic acid molecule comprising the nucleic acid SEQ ID NO. 61723, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 61723 or polypeptide SEQ ID NO. 61724, respectively, or a homolog or a fragment thereof, is increased or generated, or if the activity multiple antibiotic resistance protein is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 72 to 132-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a multiple antibiotic resistance protein, or if the activity of the polypeptide TTC1193, preferably represented by SEQ ID NO. 61724, or a homolog or fragment thereof, or if the activity of a polypeptide encoded by a nucleic acid molecule comprising the nucleic acid SEQ ID NO. 61723, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 61723 or polypeptide SEQ ID NO. 61724, respectively, or a homolog or a fragment thereof, is increased or generated, or if the activity multiple antibiotic resistance protein is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 36 to 110-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a TTC1386-protein, or if the activity of the polypeptide TTC1386, preferably represented by SEQ ID NO. 62080, or a homolog or fragment thereof, or if the activity of a polypeptide encoded by a nucleic acid molecule comprising the nucleic acid SEQ ID NO. 62079, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 62079 or polypeptide SEQ ID NO. 62080, respectively, or a homolog or a fragment thereof, is increased or generated, or if the activity TTC1386-protein is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 64 to 848-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a TTC1386-protein, or if the activity of the polypeptide TTC1386, preferably represented by SEQ ID NO. 62080, or a homolog or fragment thereof, or if the activity of a polypeptide encoded by a nucleic acid molecule comprising the nucleic acid SEQ ID NO. 62079, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 62079 or polypeptide SEQ ID NO. 62080, respectively, or a homolog or a fragment thereof, is increased or generated, or if the activity TTC1386-protein is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 241 to 353-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a TTC1386-protein, or if the activity of the polypeptide TTC1386, preferably represented by SEQ ID NO. 62080, or a homolog or fragment thereof, or if the activity of a polypeptide encoded by a nucleic acid molecule comprising the nucleic acid SEQ ID NO. 62079, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 62079 or polypeptide SEQ ID NO. 62080, respectively, or a homolog or a fragment thereof, is increased or generated, or if the activity TTC1386-protein is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 31 to 161-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a oxidoreductase subunit, or if the activity of the polypeptide TTC1918, preferably represented by SEQ ID NO. 62161, or a homolog or fragment thereof, or if the activity of a polypeptide encoded by a nucleic acid molecule comprising the nucleic acid SEQ ID NO. 62160, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 62160 or polypeptide SEQ ID NO. 62161, respectively, or a homolog or a fragment thereof, is increased or generated, or if the activity oxidoreductase subunit is increased or generated in a non-human organism, like a microorganism or a plant cell, plant or part thereof, especially with non-targeted localization, whereby the respective line disclose in column 7 the fine chemical citramalate. For example, an increase of the citramalate of at least 1 percent, particularly in a range of 43 to 80-percent is conferred as compared to a corresponding non-transformed wild type non-human organism.

Accordingly, in one embodiment, an increase of glycerate in a non-human organism, as compared to a corresponding non-transformed wild type non-human organism, is conferred in the process of the invention, if the activity of a polypeptide showing the activity of a oxidoreductase subunit, or if the activity of the polypeptide TTC1918, preferably represented by SEQ ID NO. 62161, or a homolog or fragment thereof, or if the activity of a polypeptide encoded by a nucleic acid molecule comprising the nucleic acid SEQ ID NO. 62160, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 62160 or polypeptide SEQ ID NO. 62161, respectively, or a homolog or a fragment thereof, is increased or generated, or if the activity oxidoreductase subunit is increased or generated in a non-human organism, like a microorganism or a plant cell, plant or part thereof, especially with non-targeted localization, whereby the respective line disclose in column 7 the fine chemical glycerate. For example, an increase of the glycerate of at least 1 percent, particularly in a range of 32 to 61-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a oxidoreductase subunit, or if the activity of the polypeptide TTC1918, preferably represented by SEQ ID NO. 62161, or a homolog or fragment thereof, or if the activity of a polypeptide encoded by a nucleic acid molecule comprising the nucleic acid SEQ ID NO. 62160, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 62160 or polypeptide SEQ ID NO. 62161, respectively, or a homolog or a fragment thereof, is increased or generated, or if the activity oxidoreductase subunit is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 58 to 241-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a XM_{—}473199-protein, or if the activity of the polypeptide XM_{—}473199, preferably represented by SEQ ID NO. 62245, or a homolog or fragment thereof, or if the activity of a polypeptide encoded by a nucleic acid molecule comprising the nucleic acid SEQ ID NO. 62244, preferably the coding region thereof, or a homolog or fragment thereof, e.g. derived from Oryza sativa, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 62244 or polypeptide SEQ ID NO. 62245, respectively, or a homolog or a fragment thereof, is increased or generated, or if the activity XM_{—}473199-protein is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 76 to 257-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a XM_{—}473199-protein, or if the activity of the polypeptide XM_{—}473199, preferably represented by SEQ ID NO. 62245, or a homolog or fragment thereof, or if the activity of a polypeptide encoded by a nucleic acid molecule comprising the nucleic acid SEQ ID NO. 62244, preferably the coding region thereof, or a homolog or fragment thereof, e.g. derived from Oryza sativa, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 62244 or polypeptide SEQ ID NO. 62245, respectively, or a homolog or a fragment thereof, is increased or generated, or if the activity XM_{—}473199-protein is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 54 to 129-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a XM_{—}473199protein, or if the activity of the polypeptide XM_{—}473199, preferably represented by SEQ ID NO. 62245, or a homolog or fragment thereof, or if the activity of a polypeptide encoded by a nucleic acid molecule comprising the nucleic acid SEQ ID NO. 62244, preferably the coding region thereof, or a homolog or fragment thereof, e.g. derived from Oryza sativa, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 62244 or polypeptide SEQ ID NO. 62245, respectively, or a homolog or a fragment thereof, is increased or generated, or if the activity XM_{—}473199-protein is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 31 to 75-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a glutamate dehydrogenase, or if the activity of the polypeptide Yal062w, preferably represented by SEQ ID NO. 136944, or a homolog or fragment thereof, or if the activity of a polypeptide encoded by a nucleic acid molecule comprising the nucleic acid SEQ ID NO. 136943, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 136943 or polypeptide SEQ ID NO. 136944, respectively, or a homolog or a fragment thereof, is increased or generated, or if the activity glutamate dehydrogenase is increased or generated in a non-human organism, like a microorganism or a plant cell, plant or part thereof, especially with plastidic localization, whereby the respective line disclose in column 7 the fine chemical succinate. For example, an increase of the succinate of at least 1 percent, particularly in a range of 35 to 75-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a acetyl-CoA hydrolase, or if the activity of the polypeptide Ybl015w, preferably represented by SEQ ID NO. 137388, or a homolog or fragment thereof, or if the activity of a polypeptide encoded by a nucleic acid molecule comprising the nucleic acid SEQ ID NO. 137387, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 137387 or polypeptide SEQ ID NO. 137388, respectively, or a homolog or a fragment thereof, is increased or generated, or if the activity acetyl-CoA hydrolase is increased or generated in a non-human organism, like a microorganism or a plant cell, plant or part thereof, especially with non-targeted localization, whereby the respective line disclose in column 7 the fine chemical citramalate. For example, an increase of the citramalate of at least 1 percent, particularly in a range of 39 to 127-percent is conferred as compared to a corresponding non-transformed wild type non-human organism.

Accordingly, in one embodiment, an increase of pyruvate in a non-human organism, as compared to a corresponding non-transformed wild type non-human organism, is conferred in the process of the invention, if the activity of a polypeptide showing the activity of a acetyl-CoA hydrolase, or if the activity of the polypeptide Ybl015w, preferably represented by SEQ ID NO. 137388, or a homolog or fragment thereof, or if the activity of a polypeptide encoded by a nucleic acid molecule comprising the nucleic acid SEQ ID NO. 137387, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 137387 or polypeptide SEQ ID NO. 137388, respectively, or a homolog or a fragment thereof, is increased or generated, or if the activity acetyl-CoA hydrolase is increased or generated in a non-human organism, like a microorganism or a plant cell, plant or part thereof, especially with non-targeted localization, whereby the respective line disclose in column 7 the fine chemical pyruvate. For example, an increase of the pyruvate of at least 1 percent, particularly in a range of 44 to 74-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a pyruvate carboxylase, or if the activity of the polypeptide Ybr218c, preferably represented by SEQ ID NO. 96780, or a homolog or fragment thereof, or if the activity of a polypeptide encoded by a nucleic acid molecule comprising the nucleic acid SEQ ID NO. 96779, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 96779 or polypeptide SEQ ID NO. 96780, respectively, or a homolog or a fragment thereof, is increased or generated, or if the activity pyruvate carboxylase is increased or generated in a non-human organism, like a microorganism organism or a plant cell, plant or part thereof, especially with plastidic localization, whereby the respective line disclose in column 7 the fine chemical malate. For example, an increase of the malate of at least 1 percent, particularly in a range of 49 to 99-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a ycr102c-protein, or if the activity of the polypeptide Ycr102c, preferably represented by SEQ ID NO. 63265, or a homolog or fragment thereof, or if the activity of a polypeptide encoded by a nucleic acid molecule comprising the nucleic acid SEQ ID NO. 63264, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 63264 or polypeptide SEQ ID NO. 63265, respectively, or a homolog or a fragment thereof, is increased or generated, or if the activity ycr102c-protein is increased or generated in a non-human organism, like a microorganism or a plant cell, plant or part thereof, especially with plastidic localization, whereby the respective line disclose in column 7 the fine chemical fumarate. For example, an increase of the fumarate of at least 1 percent, particularly in a range of 49 to 79-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a YDL159W-A-protein, or if the activity of the polypeptide Yd1159w-a, preferably represented by SEQ ID NO. 137499, or a homolog or fragment thereof, or if the activity of a polypeptide encoded by a nucleic acid molecule comprising the nucleic acid SEQ ID NO. 137498, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 137498 or polypeptide SEQ ID NO. 137499, respectively, or a homolog or a fragment thereof, is increased or generated, or if the activity YDL159W-A-protein is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 18 to 20-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a coproporphyrinogen III oxidase, or if the activity of the polypeptide Ydr044w, preferably represented by SEQ ID NO. 63335, or a homolog or fragment thereof, or if the activity of a polypeptide encoded by a nucleic acid molecule comprising the nucleic acid SEQ ID NO. 63334, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 63334 or polypeptide SEQ ID NO. 63335, respectively, or a homolog or a fragment thereof, is increased or generated, or if the activity coproporphyrinogen III oxidase is increased or generated in a non-human organism, like a microorganism or a plant cell, plant or part thereof, especially with plastidic localization, whereby the respective line disclose in column 7 the fine chemical fumarate. For example, an increase of the fumarate of at least 1 percent, particularly in a range of 198 to 494-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a coproporphyrinogen III oxidase, or if the activity of the polypeptide Ydr044w, preferably represented by SEQ ID NO. 63335, or a homolog or fragment thereof, or if the activity of a polypeptide encoded by a nucleic acid molecule comprising the nucleic acid SEQ ID NO. 63334, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 63334 or polypeptide SEQ ID NO. 63335, respectively, or a homolog or a fragment thereof, is increased or generated, or if the activity coproporphyrinogen III oxidase is increased or generated in a non-human organism, like a microorganism or a plant cell, plant or part thereof, especially with plastidic localization, whereby the respective line disclose in column 7 the fine chemical succinate. For example, an increase of the succinate of at least 1 percent, particularly in a range of 112 to 267-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a ygl237c-protein, or if the activity of the polypeptide Ygl237c, preferably represented by SEQ ID NO. 64178, or a homolog or fragment thereof, or if the activity of a polypeptide encoded by a nucleic acid molecule comprising the nucleic acid SEQ ID NO. 64177, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 64177 or polypeptide SEQ ID NO. 64178, respectively, or a homolog or a fragment thereof, is increased or generated, or if the activity ygl237c-protein is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 54 to 178-percent is conferred as compared to a corganism responding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a ygl237c-protein, or if the activity of the polypeptide Ygl237c, preferably represented by SEQ ID NO. 64178, or a homolog or fragment thereof, or if the activity of a polypeptide encoded by a nucleic acid molecule comprising the nucleic acid SEQ ID NO. 64177, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 64177 or polypeptide SEQ ID NO. 64178, respectively, or a homolog or a fragment thereof, is increased or generated, or if the activity ygl237c-protein is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 33 to 110-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a YGR193C-protein, or if the activity of the polypeptide Ygr193c, preferably represented by SEQ ID NO. 108488, or a homolog or fragment thereof, or if the activity of a polypeptide encoded by a nucleic acid molecule comprising the nucleic acid SEQ ID NO. 108487, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 108487 or polypeptide SEQ ID NO. 108488, respectively, or a homolog or a fragment thereof, is increased or generated, or if the activity YGR193C-protein is increased or generated in a non-human organism , like a microorganism or a plant cell, plant or part thereof, especially with non-targeted localization, whereby the respective line disclose in column 7 the fine chemical fumarate. For example, an increase of the fumarate of at least 1 percent, particularly in a range of 50 to 287-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a YGR193C-protein, or if the activity of the polypeptide Ygr193c, preferably represented by SEQ ID NO. 108488, or a homolog or fragment thereof, or if the activity of a polypeptide encoded by a nucleic acid molecule comprising the nucleic acid SEQ ID NO. 108487, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 108487 or polypeptide SEQ ID NO. 108488, respectively, or a homolog or a fragment thereof, is increased or generated, or if the activity YGR193C-protein is increased or generated in a non-human organism, like a microorganism organism or a plant cell, plant or part thereof, especially with non-targeted localization, whereby the respective line disclose in column 7 the fine chemical malate. For example, an increase of the malate of at least 1 percent, particularly in a range of 58 to 96-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a YGR193C-protein, or if the activity of the polypeptide Ygr193c, preferably represented by SEQ ID NO. 108488, or a homolog or fragment thereof, or if the activity of a polypeptide encoded by a nucleic acid molecule comprising the nucleic acid SEQ ID NO. 108487, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 108487 or polypeptide SEQ ID NO. 108488, respectively, or a homolog or a fragment thereof, is increased or generated, or if the activity YGR193C-protein is increased or generated in a non-human organism , like a microorganism or a plant cell, plant or part thereof, especially with non-targeted localization, whereby the respective line disclose in column 7 the fine chemical succinate. For example, an increase of the succinate of at least 1 percent, particularly in a range of 129 to 292-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a ygr221c-protein, or if the activity of the polypeptide Ygr221 c, preferably represented by SEQ ID NO. 64316, or a homolog or fragment thereof, or if the activity of a polypeptide encoded by a nucleic acid molecule comprising the nucleic acid SEQ ID NO. 64315, preferably the coding region thereof, or a homolog or fragment thereof, e.g. derived from Saccharomyces cerevisiae, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 64315 or polypeptide SEQ ID NO. 64316, respectively, or a homolog or a fragment thereof, is increased or generated, or if the activity ygr221c-protein is increased or generated in a non-human organism, like a microorganism or a plant cell, plant or part thereof, especially with plastidic localization, whereby the respective line disclose in column 7 the fine chemical malate. For example, an increase of the malate of at least 1 percent, particularly in a range of 59 to 80-percent is conferred as compared to a corresponding non-transformed wild type non-human organism.

Accordingly, in one embodiment, an increase of citrate in a non-human organism, as compared to a corresponding non-transformed wild type non-human organism, is conferred in the process of the invention, if the activity of a polypeptide showing the activity of a glycerol kinase, or if the activity of the polypeptide Yhl032c, preferably represented by SEQ ID NO. 101856, or a homolog or fragment thereof, or if the activity of a polypeptide encoded by a nucleic acid molecule comprising the nucleic acid SEQ ID NO. 101855, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 101855 or polypeptide SEQ ID NO. 101856, respectively, or a homolog or a fragment thereof, is increased or generated, or if the activity glycerol kinase is increased or generated in a non-human organism, like a microorganism or a plant cell, plant or part thereof, especially with plastidic localization, whereby the respective line disclose in column 7 the fine chemical citrate. For example, an increase of the citrate of at least 1 percent, particularly in a range of 73 to 311-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a yhr131c-protein, or if the activity of the polypeptide Yhr131c, preferably represented by SEQ ID NO. 97621, or a homolog or fragment thereof, or if the activity of a polypeptide encoded by a nucleic acid molecule comprising the nucleic acid SEQ ID NO. 97620, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 97620 or polypeptide SEQ ID NO. 97621, respectively, or a homolog or a fragment thereof, is increased or generated, or if the activity yhr131c-protein is increased or generated in a non-human organism, like a microorganism or a plant cell, plant or part thereof, especially with plastidic localization, whereby the respective line disclose in column 7 the fine chemical malate. For example, an increase of the malate of at least 1 percent, particularly in a range of 53 to 83-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a yhr207c-protein, or if the activity of the polypeptide Yhr207c, preferably represented by SEQ ID NO. 64547, or a homolog or fragment thereof, or if the activity of a polypeptide encoded by a nucleic acid molecule comprising the nucleic acid SEQ ID NO. 64546, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 64546 or polypeptide SEQ ID NO. 64547, respectively, or a homolog or a fragment thereof, is increased or generated, or if the activity yhr207c-protein is increased or generated in a non-human organism, like a microorganism or a plant cell, plant or part thereof, especially with plastidic localization, whereby the respective line disclose in column 7 the fine chemical succinate. For example, an increase of the succinate of at least 1 percent, particularly in a range of 59 to 166-percent is conferred as compared to a corresponding non-transformed wild type non-human organism.

Accordingly, in one embodiment, an increase of glycerate in a non-human organism, as compared to a corresponding non-transformed wild type non-human organism, is conferred in the process of the invention, if the activity of a polypeptide showing the activity of a DnaJ-like chaperone, or if the activity of the polypeptide Yjl073w, preferably represented by SEQ ID NO. 64965, or a homolog or fragment thereof, or if the activity of a polypeptide encoded by a nucleic acid molecule comprising the nucleic acid SEQ ID NO. 64964, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 64964 or polypeptide SEQ ID NO. 64965, respectively, or a homolog or a fragment thereof, is increased or generated, or if the activity DnaJ-like chaperone is increased or generated in a non-human organism, like a microorganism or a plant cell, plant or part thereof, especially with plastidic localization, whereby the respective line disclose in column 7 the fine chemical glycerate. For example, an increase of the glycerate of at least 1 percent, particularly in a range of 31 to 37-percent is conferred as compared to a corresponding non-transformed wild type non-human organism.

Accordingly, in one embodiment, an increase of oxalate in a non-human organism, as compared to a corresponding non-transformed wild type non-human organism, is conferred in the process of the invention, if the activity of a polypeptide showing the activity of a hexose transporter, or if the activity of the polypeptide Yjl219w, preferably represented by SEQ ID NO. 97636, or a homolog or fragment thereof, or if the activity of a polypeptide encoded by a nucleic acid molecule comprising the nucleic acid SEQ ID NO. 97635, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 97635 or polypeptide SEQ ID NO. 97636, respectively, or a homolog or a fragment thereof, is increased or generated, or if the activity hexose transporter is increased or generated in a non-human organism, like a microorganism or a plant cell, plant or part thereof, especially with non-targeted localization, whereby the respective line disclose in column 7 the fine chemical oxalate. For example, an increase of the oxalate of at least 1 percent, particularly in a range of 33 to 194-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a branched chain aminotransferase, or if the activity of the polypeptide Yjr148w, preferably represented by SEQ ID NO. 90410, or a homolog or fragment thereof, or if the activity of a polypeptide encoded by a nucleic acid molecule comprising the nucleic acid SEQ ID NO. 90409, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 90409 or polypeptide SEQ ID NO. 90410, respectively, or a homolog or a fragment thereof, is increased or generated, or if the activity branched chain aminotransferase is increased or generated in a non-human organism, like a microorganism or a plant cell, plant or part thereof, especially with plastidic localization, whereby the respective line disclose in column 7 the fine chemical citramalate. For example, an increase of the citramalate of at least 1 percent, particularly in a range of 97 to 244-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a polygalacturonase, or if the activity of the polypeptide Yjr153w, preferably represented by SEQ ID NO. 66275, or a homolog or fragment thereof, or if the activity of a polypeptide encoded by a nucleic acid molecule comprising the nucleic acid SEQ ID NO. 66274, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 66274 or polypeptide SEQ ID NO. 66275, respectively, or a homolog or a fragment thereof, is increased or generated, or if the activity polygalacturonase is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 50 to 138-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a polygalacturonase, or if the activity of the polypeptide Yjr153w, preferably represented by SEQ ID NO. 66275, or a homolog or fragment thereof, or if the activity of a polypeptide encoded by a nucleic acid molecule comprising the nucleic acid SEQ ID NO. 66274, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 66274 or polypeptide SEQ ID NO. 66275, respectively, or a homolog or a fragment thereof, is increased or generated, or if the activity polygalacturonase is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 63 to 166-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a YKL038W-protein, or if the activity of the polypeptide YKL038W, preferably represented by SEQ ID NO. 90603, or a homolog or fragment thereof, or if the activity of a polypeptide encoded by a nucleic acid molecule comprising the nucleic acid SEQ ID NO. 90602, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 90602 or polypeptide SEQ ID NO. 90603, respectively, or a homolog or a fragment thereof, is increased or generated, or if the activity YKL038W-protein is increased or generated in a non-human organism, like a microorganism or a plant cell, plant or part thereof, especially with plastidic localization, whereby the respective line disclose in column 7 the fine chemical malate. For example, an increase of the malate of at least 1 percent, particularly in a range of 67 to 159-percent is conferred as compared to a corresponding non-transformed wild type non-human organism.

Accordingly, in one embodiment, an increase of vitamin c and/or threonic acid in a non-human organism, as compared to a corresponding non-transformed wild type non-human organism, is conferred in the process of the invention, if the activity of a polypeptide showing the activity of a YKL140W-protein, or if the activity of the polypeptide Ykl140w, preferably represented by SEQ ID NO. 137503, or a homolog or fragment thereof, or if the activity of a polypeptide encoded by a nucleic acid molecule comprising the nucleic acid SEQ ID NO. 137502, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 137502 or polypeptide SEQ ID NO. 137503, respectively, or a homolog or a fragment thereof, is increased or generated, or if the activity YKL140W-protein is increased or generated in a non-human organism , like a microorganism or a plant cell, plant or part thereof, especially with non-targeted localization, whereby the respective line disclose in column 7 the fine chemical vitamin c and/or threonic acid. For example, an increase of the vitamin c and/or threonic acid of at least 1 percent, particularly in a range of 39 to 145-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a ylr065c-protein, or if the activity of the polypeptide Ylr065c, preferably represented by SEQ ID NO. 66696, or a homolog or fragment thereof, or if the activity of a polypeptide encoded by a nucleic acid molecule comprising the nucleic acid SEQ ID NO. 66695, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 66695 or polypeptide SEQ ID NO. 66696, respectively, or a homolog or a fragment thereof, is increased or generated, or if the activity ylr065c-protein is increased or generated in a non-human organism, like a microorganism or a plant cell, plant or part thereof, especially with plastidic localization, whereby the respective line disclose in column 7 the fine chemical fumarate. For example, an increase of the fumarate of at least 1 percent, particularly in a range of 33 to 45-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a isocitrate dehydrogenase, or if the activity of the polypeptide Ylr174w, preferably represented by SEQ ID NO. 137548, or a homolog or fragment thereof, or if the activity of a polypeptide encoded by a nucleic acid molecule comprising the nucleic acid SEQ ID NO. 137547, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 137547 or polypeptide SEQ ID NO. 137548, respectively, or a homolog or a fragment thereof, is increased or generated, or if the activity isocitrate dehydrogenase is increased or generated in a non-human organism, like a microorganism or a plant cell, plant or part thereof, especially with plastidic localization, whereby the respective line disclose in column 7 the fine chemical succinate. For example, an increase of the succinate of at least 1 percent, particularly in a range of 29 to 48-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a ylr326w-protein, or if the activity of the polypeptide Ylr326w, preferably represented by SEQ ID NO. 129124, or a homolog or fragment thereof, or if the activity of a polypeptide encoded by a nucleic acid molecule comprising the nucleic acid SEQ ID NO. 129123, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 129123 or polypeptide SEQ ID NO. 129124, respectively, or a homolog or a fragment thereof, is increased or generated, or if the activity ylr326w-protein is increased or generated in a non-human organism, like a microorganism or a plant cell, plant or part thereof, especially with plastidic localization, whereby the respective line disclose in column 7 the fine chemical fumarate. For example, an increase of the fumarate of at least 1 percent, particularly in a range of 71 to 254-percent is conferred as compared to a corresponding non-transformed wild type non-human organism.

Accordingly, in one embodiment, an increase of glycerate in a non-human organism, as compared to a corresponding non-transformed wild type non-human organism, is conferred in the process of the invention, if the activity of a polypeptide showing the activity of a ylr326w-protein, or if the activity of the polypeptide Ylr326w, preferably represented by SEQ ID NO. 129124, or a homolog or fragment thereof, or if the activity of a polypeptide encoded by a nucleic acid molecule comprising the nucleic acid SEQ ID NO. 129123, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 129123 or polypeptide SEQ ID NO. 129124, respectively, or a homolog or a fragment thereof, is increased or generated, or if the activity ylr326w-protein is increased or generated in a non-human organism, like a microorganism or a plant cell, plant or part thereof, especially with plastidic localization, whereby the respective line disclose in column 7 the fine chemical glycerate. For example, an increase of the glycerate of at least 1 percent, particularly in a range of 37 to 72-percent is conferred as compared to a corresponding non-transformed wild type non-human organism.

Accordingly, in one embodiment, an increase of vitamin c and/or threonic acid in a non-human organism, as compared to a corresponding non-transformed wild type non-human organism, is conferred in the process of the invention, if the activity of a polypeptide showing the activity of a ylr326w-protein, or if the activity of the polypeptide Ylr326w, preferably represented by SEQ ID NO. 129124, or a homolog or fragment thereof, or if the activity of a polypeptide encoded by a nucleic acid molecule comprising the nucleic acid SEQ ID NO. 129123, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 129123 or polypeptide SEQ ID NO. 129124, respectively, or a homolog or a fragment thereof, is increased or generated, or if the activity ylr326w-protein is increased or generated in a non-human organism, like a microorganism or a plant cell, plant or part thereof, especially with plastidic localization, whereby the respective line disclose in column 7 the fine chemical vitamin c and/or threonic acid. For example, an increase of the vitamin c and/or threonic acid of at least 1 percent, particularly in a range of 30 to 33-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a yml084w-protein, or if the activity of the polypeptide Yml084w, preferably represented by SEQ ID NO. 15176, or a homolog or fragment thereof, or if the activity of a polypeptide encoded by a nucleic acid molecule comprising the nucleic acid SEQ ID NO. 15175, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 15175 or polypeptide SEQ ID NO. 15176, respectively, or a homolog or a fragment thereof, is increased or generated, or if the activity yml084w-protein is increased or generated in a non-human organism, like a microorganism or a plant cell, plant or part thereof, especially with plastidic localization, whereby the respective line disclose in column 7 the fine chemical fumarate. For example, an increase of the fumarate of at least 1 percent, particularly in a range of 30 to 80-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a yml084w-protein, or if the activity of the polypeptide Yml084w, preferably represented by SEQ ID NO. 15176, or a homolog or fragment thereof, or if the activity of a polypeptide encoded by a nucleic acid molecule comprising the nucleic acid SEQ ID NO. 15175, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 15175 or polypeptide SEQ ID NO. 15176, respectively, or a homolog or a fragment thereof, is increased or generated, or if the activity yml084w-protein is increased or generated in a non-human organism, like a microorganism or a plant cell, plant or part thereof, especially with plastidic localization, whereby the respective line disclose in column 7 the fine chemical succinate. For example, an increase of the succinate of at least 1 percent, particularly in a range of 53 to 165-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a yol073c-protein, or if the activity of the polypeptide Yol073c, preferably represented by SEQ ID NO. 137819, or a homolog or fragment thereof, or if the activity of a polypeptide encoded by a nucleic acid molecule comprising the nucleic acid SEQ ID NO. 137818, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 137818 or polypeptide SEQ ID NO. 137819, respectively, or a homolog or a fragment thereof, is increased or generated, or if the activity yol073c-protein is increased or generated in a non-human organism, like a microorganism or a plant cell, plant or part thereof, especially with plastidic localization, whereby the respective line disclose in column 7 the fine chemical citramalate. For example, an increase of the citramalate of at least 1 percent, particularly in a range of 43 to 59-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a yol073c-protein, or if the activity of the polypeptide Yol073c, preferably represented by SEQ ID NO. 137819, or a homolog or fragment thereof, or if the activity of a polypeptide encoded by a nucleic acid molecule comprising the nucleic acid SEQ ID NO. 137818, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 137818 or polypeptide SEQ ID NO. 137819, respectively, or a homolog or a fragment thereof, is increased or generated, or if the activity yol073c-protein is increased or generated in a non-human organism, like a microorganism or a plant cell, plant or part thereof, especially with plastidic localization, whereby the respective line disclose in column 7 the fine chemical malate. For example, an increase of the malate of at least 1 percent, particularly in a range of 62 to 188-percent is conferred as compared to a corresponding non-transformed wild type non-human organism.

Accordingly, in one embodiment, an increase of vitamin c and/or threonic acid in a non-human organism, as compared to a corresponding non-transformed wild type non-human organism, is conferred in the process of the invention, if the activity of a polypeptide showing the activity of a yol160w-protein, or if the activity of the polypeptide Yol160w, preferably represented by SEQ ID NO. 15180, or a homolog or fragment thereof, or if the activity of a polypeptide encoded by a nucleic acid molecule comprising the nucleic acid SEQ ID NO. 15179, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 15179 or polypeptide SEQ ID NO. 15180, respectively, or a homolog or a fragment thereof, is increased or generated, or if the activity yol160w-protein is increased or generated in a non-human organism, like a microorganism or a plant cell, plant or part thereof, especially with plastidic localization, whereby the respective line disclose in column 7 the fine chemical vitamin c and/or threonic acid. For example, an increase of the vitamin c and/or threonic acid of at least 1 percent, particularly in a range of 28 to 43-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a acetyl-CoA acetyltransferase, or if the activity of the polypeptide Ypl028w, preferably represented by SEQ ID NO. 92669, or a homolog or fragment thereof, or if the activity of a polypeptide encoded by a nucleic acid molecule comprising the nucleic acid SEQ ID NO. 92668, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 92668 or polypeptide SEQ ID NO. 92669, respectively, or a homolog or a fragment thereof, is increased or generated, or if the activity acetyl-CoA acetyltransferase is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 41 to 88-percent is conferred as compared to a corresponding non-transformed wild type non-human organism.

Accordingly, in one embodiment, an increase of vitamin c and/or threonic acid in a non-human organism, as compared to a corresponding non-transformed wild type non-human organism, is conferred in the process of the invention, if the activity of a polypeptide showing the activity of a ammonium transporter, or if the activity of the polypeptide Ypr138c, preferably represented by SEQ ID NO. 93219, or a homolog or fragment thereof, or if the activity of a polypeptide encoded by a nucleic acid molecule comprising the nucleic acid SEQ ID NO. 93218, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 93218 or polypeptide SEQ ID NO. 93219, respectively, or a homolog or a fragment thereof, is increased or generated, or if the activity ammonium transporter is increased or generated in a non-human organism, like a microorganism or a plant cell, plant or part thereof, especially with non-targeted localization, whereby the respective line disclose in column 7 the fine chemical vitamin c and/or threonic acid. For example, an increase of the vitamin c and/or threonic acid of at least 1 percent, particularly in a range of 37 to 125-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a 47266012protein, or if the activity of the polypeptide 47266012_SOYBEAN, preferably represented by SEQ ID NO. 15188, or a homolog or fragment thereof, or if the activity of a polypeptide encoded by a nucleic acid molecule comprising the nucleic acid SEQ ID NO. 15187, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 15187 or polypeptide SEQ ID NO. 15188, respectively, or a homolog or a fragment thereof, is increased or generated, or if the activity 47266012-protein is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 75 to 378-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a 47266012-protein, or if the activity of the polypeptide 47266012_SOYBEAN, preferably represented by SEQ ID NO. 15188, or a homolog or fragment thereof, or if the activity of a polypeptide encoded by a nucleic acid molecule comprising the nucleic acid SEQ ID NO. 15187, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 15187 or polypeptide SEQ ID NO. 15188, respectively, or a homolog or a fragment thereof, is increased or generated, or if the activity 47266012-protein is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 59 to 201-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a 47266012-protein, or if the activity of the polypeptide 47266012_SOYBEAN, preferably represented by SEQ ID NO. 15188, or a homolog or fragment thereof, or if the activity of a polypeptide encoded by a nucleic acid molecule comprising the nucleic acid SEQ ID NO. 15187, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 15187 or polypeptide SEQ ID NO. 15188, respectively, or a homolog or a fragment thereof, is increased or generated, or if the activity 47266012-protein is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 46 to 112-percent is conferred as compared to a corresponding non-transformed wild type non-human organism.

Accordingly, in one embodiment, an increase of fumarate in a non-human organism, as compared to a corresponding non-transformed wild type non-human organism, is conferred in the process of the invention, if the activity of a polypeptide showing the activity of a 49747384_SOYBEAN-protein, or if the activity of the polypeptide 49747384_SOYBEAN, preferably represented by SEQ ID NO. 70, or a homolog or fragment thereof, or if the activity of a polypeptide encoded by a nucleic acid molecule comprising the nucleic acid SEQ ID NO. 69, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 69 or polypeptide SEQ ID NO. 70, respectively, or a homolog or a fragment thereof, is increased or generated, or if the activity 49747384_SOYBEAN-protein is increased or generated in a non-human organism, like a microorganism or a plant cell, plant or part thereof, especially with non-targeted localization, whereby the respective line disclose in column 7 the fine chemical fumarate. For example, an increase of the fumarate of at least 1 percent, particularly in a range of 100 to 340-percent is conferred as compared to a corresponding non-transformed wild type non-human organism.

Accordingly, in one embodiment, an increase of malate in a non-human organism, as compared to a corresponding non-transformed wild type non-human organism, is conferred in the process of the invention, if the activity of a polypeptide showing the activity of a 49747384_SOYBEAN-protein, or if the activity of the polypeptide 49747384_SOYBEAN, preferably represented by SEQ ID NO. 70, or a homolog or fragment thereof, or if the activity of a polypeptide encoded by a nucleic acid molecule comprising the nucleic acid SEQ ID NO. 69, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 69 or polypeptide SEQ ID NO. 70, respectively, or a homolog or a fragment thereof, is increased or generated, or if the activity 49747384_SOYBEAN-protein is increased or generated in a non-human organism, like a microorganism or a plant cell, plant or part thereof, especially with non-targeted localization, whereby the respective line disclose in column 7 the fine chemical malate. For example, an increase of the malate of at least 1 percent, particularly in a range of 54 to 202-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a 49747384_SOYBEAN-protein, or if the activity of the polypeptide 49747384_SOYBEAN, preferably represented by SEQ ID NO. 70, or a homolog or fragment thereof, or if the activity of a polypeptide encoded by a nucleic acid molecule comprising the nucleic acid SEQ ID NO. 69, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 69 or polypeptide SEQ ID NO. 70, respectively, or a homolog or a fragment thereof, is increased or generated, or if the activity 49747384_SOYBEAN-protein is increased or generated in a non-human organism, like a microorganism or a plant cell, plant or part thereof, especially with non-targeted localization, whereby the respective line disclose in column 7 the fine chemical succinate. For example, an increase of the succinate of at least 1 percent, particularly in a range of 46 to 129-percent is conferred as compared to a corresponding non-transformed wild type non-human organism.

Accordingly, in one embodiment, an increase of vitamin c and/or threonic acid in a non-human organism, as compared to a corresponding non-transformed wild type non-human organism, is conferred in the process of the invention, if the activity of a polypeptide showing the activity of a 49747384_SOYBEAN-protein, or if the activity of the polypeptide 49747384_SOYBEAN, preferably represented by SEQ ID NO. 70, or a homolog or fragment thereof, or if the activity of a polypeptide encoded by a nucleic acid molecule comprising the nucleic acid SEQ ID NO. 69, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 69 or polypeptide SEQ ID NO. 70, respectively, or a homolog or a fragment thereof, is increased or generated, or if the activity 49747384_SOYBEAN-protein is increased or generated in a non-human organism, like a microorganism or a plant cell, plant or part thereof, especially with non-targeted localization, whereby the respective line disclose in column 7 the fine chemical vitamin c and/or threonic acid. For example, an increase of the vitamin c and/or threonic acid of at least 1 percent, particularly in a range of 32 to 64-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a pyruvate kinase, or if the activity of the polypeptide 51340801_CANOLA, preferably represented by SEQ ID NO. 15533, or a homolog or fragment thereof, or if the activity of a polypeptide encoded by a nucleic acid molecule comprising the nucleic acid SEQ ID NO. 15532, preferably the coding region thereof, or a homolog or fragment thereof, e.g. derived from Brassica napus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 15532 or polypeptide SEQ ID NO. 15533, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line disclose in column 7 the fine chemical citramalate. For example, an increase of the citramalate of at least 1 percent, particularly in a range of 49 to 420-percent is conferred as compared to a corresponding non-transformed wild type non-human organism.

Accordingly, in one embodiment, an increase of succinate in a non-human organism, as compared to a corresponding non-transformed wild type non-human organism, is conferred in the process of the invention, if the activity of a polypeptide showing the activity of a pyruvate kinase, or if the activity of the polypeptide 51340801_CANOLA, preferably represented by SEQ ID NO. 15533, or a homolog or fragment thereof, or if the activity of a polypeptide encoded by a nucleic acid molecule comprising the nucleic acid SEQ ID NO. 15532, preferably the coding region thereof, or a homolog or fragment thereof, e.g. derived from Brassica napus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 15532 or polypeptide SEQ ID NO. 15533, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line disclose in column 7 the fine chemical succinate. For example, an increase of the succinate of at least 1 percent, particularly in a range of 31 to 210-percent is conferred as compared to a corresponding non-transformed wild type non-human organism.

Accordingly, in one embodiment, an increase of citramalate in a non-human organism, as compared to a corresponding non-transformed wild type non-human organism, is conferred in the process of the invention, if the activity of a polypeptide showing the activity of a glucose-6-phosphate 1-dehydrogenase, or if the activity of the polypeptide 59582753_SOYBEAN, preferably represented by SEQ ID NO. 16884, or a homolog or fragment thereof, or if the activity of a polypeptide encoded by a nucleic acid molecule comprising the nucleic acid SEQ ID NO. 16883, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 15, column 5 or 8 in the respective same line as the nucleic acid molecule SEQ ID NO. 16883 or polypeptide SEQ ID NO. 16884, respectively, or a homolog or a fragment thereof, is increased or generated, or if the activity glucose-6-phosphate 1-dehydrogenase is increased or generated in a non-human organism, like a microorganism or a plant cell, plant or part thereof, especially with plastidic localization, whereby the respective line disclose in column 7 the fine chemical citramalate. For example, an increase of the citramalate of at least 1 percent, particularly in a range of 70 to 246-percent is conferred as compared to a corresponding non-transformed wild type non-human organism.

[0096.1.7.15] to [0103.1.7.15] for the disclosure of these paragraphs see [0096.1.7.7] to [0103.1.7.7] above

In this paragraph “A” means “Goffeau et al., Science 274 (5287), 546 (1996)”, “B” means “Blattner et al., Science 277 (5331), 1453 (1997)”, “C” means “Nakamura et al., Nucleic Acids Res. 28(1), 72 (2000)”, “D” means “deposition of the sequences to the EMBL/GenBank/DDBJ databases under “Sequencing of the draft genome assembly of Azotobacter vinelandii AvOP”. in June 2005 by Copeland et al.”, “E” means “Henne et al., Nat. Biotechnol. 22 (5), 547 (2004)”, “F” means “Kaul et al., Nature 408 (6814), 796 (2000)”, and “G” means “Matsumoto et al., Nature 436 (7052), 793 (2005)”.

The nucleic acid sequence of At1g07430 from Arabidopsis thaliana, e.g. as shown in the respective line in column 5 of Table I, application no. 15, has been published in F. And the activity of the gene product thereof is the activity of protein phosphatase.

Accordingly, in one embodiment, the process of the present invention for producing succinate in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said At1g07430, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At1g07430, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At1g07430, or a functional equivalent or a homolog thereof as depicted in column 8 of Table II, application no. 15, preferably a homolog or functional equivalent as depicted in column 8 of Table II B, application no. 15, and being depicted in the same respective line as said At1g07430, and preferably the activity is increased non-targeted, whereby the respective line disclose in column 7 the fine chemical succinate.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 17451, preferably the coding region thereof, conferred the production of or the increase in succinate compared with the wild type control.

The nucleic acid sequence of At1g13930 from Arabidopsis thaliana, e.g. as shown in the respective line in column 5 of Table I, application no. 15, has been published in F. And the activity of the gene product thereof is the activity of At1g13930-protein.

Accordingly, in one embodiment, the process of the present invention for producing malate in a non-human organism, like a microorganism or a plant or a part thereof, comprises increasing or generating the activity of a gene product with the activity of a gene product conferring the activity of “At1g13930-protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said At1g13930, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At1g13930, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At1g13930, or a functional equivalent or a homolog thereof as depicted in column 8 of Table II, application no. 15, preferably a homolog or functional equivalent as depicted in column 8 of Table II B, application no. 15, and being depicted in the same respective line as said At1g13930, and preferably the activity is increased non-targeted, whereby the respective line disclose in column 7 the fine chemical malate.

Accordingly, in one embodiment, the molecule which activity is to be increased in the process of the invention is the gene product with an activity as a “At1g13930-protein”, preferably it is the molecule of section (a) or (b) of this paragraph.

In particular, it was observed that in plants, especially in Arabidopsis thaliana, increasing or generating the activity of a gene product non-targeted with the activity of a “At1g13930-protein”, preferably being encoded by a gene comprising the nucleic acid sequence SEQ ID NO. 133001, preferably the coding region thereof, conferred the production of or the increase in malate compared with the wild type control.

The nucleic acid sequence of At1g13930 from Arabidopsis thaliana, e.g. as shown in the respective line in column 5 of Table I, application no. 15, has been published in F. And the activity of the gene product thereof is the activity of At1g13930-protein.

Accordingly, in one embodiment, the process of the present invention for producing succinate in a non-human organism, like a microorganism or a plant or a part thereof, comprises increasing or generating the activity of a gene product with the activity of a gene product conferring the activity of “At1g13930-protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said At1g13930, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At1g13930, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At1g13930, or a functional equivalent or a homolog thereof as depicted in column 8 of Table II, application no. 15, preferably a homolog or functional equivalent as depicted in column 8 of Table II B, application no. 15, and being depicted in the same respective line as said At1g13930, and preferably the activity is increased non-targeted, whereby the respective line disclose in column 7 the fine chemical succinate.

Accordingly, in one embodiment, the molecule which activity is to be increased in the process of the invention is the gene product with an activity as a “At1g13930-protein”, preferably it is the molecule of section (a) or (b) of this paragraph.

In particular, it was observed that in plants, especially in Arabidopsis thaliana, increasing or generating the activity of a gene product non-targeted with the activity of a “At1g13930-protein”, preferably being encoded by a gene comprising the nucleic acid sequence SEQ ID NO. 133001, preferably the coding region thereof, conferred the production of or the increase in succinate compared with the wild type control.

The nucleic acid sequence of At1g36730 from Arabidopsis thaliana, e.g. as shown in the respective line in column 5 of Table I, application no. 15, has been published in F. And the activity of the gene product thereof is the activity of eukaryotic translation initiation factor 5.

Accordingly, in one embodiment, the process of the present invention for producing malate in a non-human organism, like a microorganism or a plant or a part thereof, comprises increasing or generating the activity of a gene product with the activity of a gene product conferring the activity of “eukaryotic translation initiation factor 5”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said At1g36730, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At1g36730, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At1g36730, or a functional equivalent or a homolog thereof as depicted in column 8 of Table II, application no. 15, preferably a homolog or functional equivalent as depicted in column 8 of Table II B, application no. 15, and being depicted in the same respective line as said At1g36730, and preferably the activity is increased non-targeted, whereby the respective line disclose in column 7 the fine chemical malate.

Accordingly, in one embodiment, the molecule which activity is to be increased in the process of the invention is the gene product with an activity as a “eukaryotic translation initiation factor 5”, preferably it is the molecule of section (a) or (b) of this paragraph.

In particular, it was observed that in plants, especially in Arabidopsis thaliana, increasing or generating the activity of a gene product non-targeted with the activity of a “eukaryotic translation initiation factor 5”, preferably being encoded by a gene comprising the nucleic acid sequence SEQ ID NO. 17968, preferably the coding region thereof, conferred the production of or the increase in malate compared with the wild type control.

The nucleic acid sequence of At1g36730 from Arabidopsis thaliana, e.g. as shown in the respective line in column 5 of Table I, application no. 15, has been published in F. And the activity of the gene product thereof is the activity of eukaryotic translation initiation factor 5.

Accordingly, in one embodiment, the process of the present invention for producing vitamin c and/or threonic acid in a non-human organism, like a microorganism or a plant or a part thereof, comprises increasing or generating the activity of a gene product with the activity of a gene product conferring the activity of “eukaryotic translation initiation factor 5”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical vitamin c and/or threonic acid), application no. 15, preferably the coding region thereof, or a homolog or a fragment thereof, and being depicted in the same respective line as said At1g36730, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At1g36730, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At1g36730, or a functional equivalent or a homolog thereof as depicted in column 8 of Table II, application no. 15, preferably a homolog or functional equivalent as depicted in column 8 of Table II B, application no. 15, and being depicted in the same respective line as said At1g36730, and preferably the activity is increased non-targeted, whereby the respective line disclose in column 7 the fine chemical vitamin c and/or threonic acid.

Accordingly, in one embodiment, the molecule which activity is to be increased in the process of the invention is the gene product with an activity as a “eukaryotic translation initiation factor 5”, preferably it is the molecule of section (a) or (b) of this paragraph.

In particular, it was observed that in plants, especially in Arabidopsis thaliana, increasing or generating the activity of a gene product non-targeted with the activity of a “eukaryotic translation initiation factor 5”, preferably being encoded by a gene comprising the nucleic acid sequence SEQ ID NO. 17968, preferably the coding region thereof, conferred the production of or the increase in vitamin c and/or threonic acid compared with the wild type control.

The nucleic acid sequence of At1g43850 from Arabidopsis thaliana, e.g. as shown in the respective line in column 5 of Table I, application no. 15, has been published in F. And the activity of the gene product thereof is the activity of transcriptional regulator.

Accordingly, in one embodiment, the process of the present invention for producing fumarate in a non-human organism, like a microorganism or a plant or a part thereof, comprises increasing or generating the activity of a gene product with the activity of a gene product conferring the activity of “transcriptional regulator”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said At1g43850, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At1g43850, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At1g43850, or a functional equivalent or a homolog thereof as depicted in column 8 of Table II, application no. 15, preferably a homolog or functional equivalent as depicted in column 8 of Table II B, application no. 15, and being depicted in the same respective line as said At1g43850, and preferably the activity is increased non-targeted, whereby the respective line disclose in column 7 the fine chemical fumarate.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 18070, preferably the coding region thereof, conferred the production of or the increase in fumarate compared with the wild type control.

The nucleic acid sequence of At1g48040 from Arabidopsis thaliana, e.g. as shown in the respective line in column 5 of Table I, application no. 15, has been published in F. And the activity of the gene product thereof is the activity of protein phosphatase.

Accordingly, in one embodiment, the process of the present invention for producing fumarate in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said At1g48040, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At1g48040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At1g48040, or a functional equivalent or a homolog thereof as depicted in column 8 of Table II, application no. 15, preferably a homolog or functional equivalent as depicted in column 8 of Table II B, application no. 15, and being depicted in the same respective line as said At1g48040, and preferably the activity is increased non-targeted, whereby the respective line disclose in column 7 the fine chemical fumarate.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 812, preferably the coding region thereof, conferred the production of or the increase in fumarate compared with the wild type control.

The nucleic acid sequence of At1g72770 from Arabidopsis thaliana, e.g. as shown in the respective line in column 5 of Table I, application no. 15, has been published in F. And the activity of the gene product thereof is the activity of protein phosphatase.

Accordingly, in one embodiment, the process of the present invention for producing vitamin c and/or threonic acid in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical vitamin c and/or threonic acid), application no. 15, preferably the coding region thereof, or a homolog or a fragment thereof, and being depicted in the same respective line as said At1g72770, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At1g72770, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At1g72770, or a functional equivalent or a homolog thereof as depicted in column 8 of Table II, application no. 15, preferably a homolog or functional equivalent as depicted in column 8 of Table II B, application no. 15, and being depicted in the same respective line as said At1g72770, and preferably the activity is increased non-targeted, whereby the respective line disclose in column 7 the fine chemical vitamin c and/or threonic acid.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19419, preferably the coding region thereof, conferred the production of or the increase in vitamin c and/or threonic acid compared with the wild type control.

The nucleic acid sequence of At2g30360 from Arabidopsis thaliana, e.g. as shown in the respective line in column 5 of Table I, application no. 15, has been published in F. And the activity of the gene product thereof is the activity of CBL-interacting protein kinase.

Accordingly, in one embodiment, the process of the present invention for producing fumarate in a non-human organism, like a microorganism or a plant or a part thereof, comprises increasing or generating the activity of a gene product with the activity of a gene product conferring the activity of “CBL-interacting protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said At2g30360, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At2g30360, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At2g30360, or a functional equivalent or a homolog thereof as depicted in column 8 of Table II, application no. 15, preferably a homolog or functional equivalent as depicted in column 8 of Table II B, application no. 15, and being depicted in the same respective line as said At2g30360, and preferably the activity is increased non-targeted, whereby the respective line disclose in column 7 the fine chemical fumarate.

Accordingly, in one embodiment, the molecule which activity is to be increased in the process of the invention is the gene product with an activity as a “CBL-interacting protein kinase”, preferably it is the molecule of section (a) or (b) of this paragraph.

In particular, it was observed that in plants, especially in Arabidopsis thaliana, increasing or generating the activity of a gene product non-targeted with the activity of a “CBL-interacting protein kinase”, preferably being encoded by a gene comprising the nucleic acid sequence SEQ ID NO. 19919, preferably the coding region thereof, conferred the production of or the increase in fumarate compared with the wild type control.

The nucleic acid sequence of At2g30360 from Arabidopsis thaliana, e.g. as shown in the respective line in column 5 of Table I, application no. 15, has been published in F. And the activity of the gene product thereof is the activity of CBL-interacting protein kinase.

Accordingly, in one embodiment, the process of the present invention for producing malate in a non-human organism, like a microorganism or a plant or a part thereof, comprises increasing or generating the activity of a gene product with the activity of a gene product conferring the activity of “CBL-interacting protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said At2g30360, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At2g30360, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At2g30360, or a functional equivalent or a homolog thereof as depicted in column 8 of Table II, application no. 15, preferably a homolog or functional equivalent as depicted in column 8 of Table II B, application no. 15, and being depicted in the same respective line as said At2g30360, and preferably the activity is increased non-targeted, whereby the respective line disclose in column 7 the fine chemical malate.

Accordingly, in one embodiment, the molecule which activity is to be increased in the process of the invention is the gene product with an activity as a “CBL-interacting protein kinase”, preferably it is the molecule of section (a) or (b) of this paragraph.

In particular, it was observed that in plants, especially in Arabidopsis thaliana, increasing or generating the activity of a gene product non-targeted with the activity of a “CBL-interacting protein kinase”, preferably being encoded by a gene comprising the nucleic acid sequence SEQ ID NO. 19919, preferably the coding region thereof, conferred the production of or the increase in malate compared with the wild type control.

The nucleic acid sequence of At2g30360 from Arabidopsis thaliana, e.g. as shown in the respective line in column 5 of Table I, application no. 15, has been published in F. And the activity of the gene product thereof is the activity of CBL-interacting protein kinase.

Accordingly, in one embodiment, the process of the present invention for producing succinate in a non-human organism, like a microorganism or a plant or a part thereof, comprises increasing or generating the activity of a gene product with the activity of a gene product conferring the activity of “CBL-interacting protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said At2g30360, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At2g30360, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At2g30360, or a functional equivalent or a homolog thereof as depicted in column 8 of Table II, application no. 15, preferably a homolog or functional equivalent as depicted in column 8 of Table II B, application no. 15, and being depicted in the same respective line as said At2g30360, and preferably the activity is increased non-targeted, whereby the respective line disclose in column 7 the fine chemical succinate.

Accordingly, in one embodiment, the molecule which activity is to be increased in the process of the invention is the gene product with an activity as a “CBL-interacting protein kinase”, preferably it is the molecule of section (a) or (b) of this paragraph.

In particular, it was observed that in plants, especially in Arabidopsis thaliana, increasing or generating the activity of a gene product non-targeted with the activity of a “CBL-interacting protein kinase”, preferably being encoded by a gene comprising the nucleic acid sequence SEQ ID NO. 19919, preferably the coding region thereof, conferred the production of or the increase in succinate compared with the wild type control.

The nucleic acid sequence of At2g37040 from Arabidopsis thaliana, e.g. as shown in the respective line in column 5 of Table I, application no. 15, has been published in F. And the activity of the gene product thereof is the activity of phenylalanine ammonia-lyase.

Accordingly, in one embodiment, the process of the present invention for producing fumarate in a non-human organism, like a microorganism or a plant or a part thereof, comprises increasing or generating the activity of a gene product with the activity of a gene product conferring the activity of “phenylalanine ammonia-lyase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said At2g37040, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At2g37040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At2g37040, or a functional equivalent or a homolog thereof as depicted in column 8 of Table II, application no. 15, preferably a homolog or functional equivalent as depicted in column 8 of Table II B, application no. 15, and being depicted in the same respective line as said At2g37040, and preferably the activity is increased non-targeted, whereby the respective line disclose in column 7 the fine chemical fumarate.

Accordingly, in one embodiment, the molecule which activity is to be increased in the process of the invention is the gene product with an activity as a “phenylalanine ammonia-lyase”, preferably it is the molecule of section (a) or (b) of this paragraph.

In particular, it was observed that in plants, especially in Arabidopsis thaliana, increasing or generating the activity of a gene product non-targeted with the activity of a “phenylalanine ammonia-lyase”, preferably being encoded by a gene comprising the nucleic acid sequence SEQ ID NO. 133042, preferably the coding region thereof, conferred the production of or the increase in fumarate compared with the wild type control.

The nucleic acid sequence of At2g41680 from Arabidopsis thaliana, e.g. as shown in the respective line in column 5 of Table I, application no. 15, has been published in F. And the activity of the gene product thereof is the activity of thioredoxin reductase.

Accordingly, in one embodiment, the process of the present invention for producing succinate in a non-human organism, like a microorganism or a plant or a part thereof, comprises increasing or generating the activity of a gene product with the activity of a gene product conferring the activity of “thioredoxin reductase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said At2g41680, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At2g41680, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At2g41680, or a functional equivalent or a homolog thereof as depicted in column 8 of Table II, application no. 15, preferably a homolog or functional equivalent as depicted in column 8 of Table II B, application no. 15, and being depicted in the same respective line as said At2g41680, and preferably the activity is increased non-targeted, whereby the respective line disclose in column 7 the fine chemical succinate.

Accordingly, in one embodiment, the molecule which activity is to be increased in the process of the invention is the gene product with an activity as a “thioredoxin reductase”, preferably it is the molecule of section (a) or (b) of this paragraph.

In particular, it was observed that in plants, especially in Arabidopsis thaliana, increasing or generating the activity of a gene product non-targeted with the activity of a “thioredoxin reductase”, preferably being encoded by a gene comprising the nucleic acid sequence SEQ ID NO. 133349, preferably the coding region thereof, conferred the production of or the increase in succinate compared with the wild type control.

The nucleic acid sequence of At2g44350 from Arabidopsis thaliana, e.g. as shown in the respective line in column 5 of Table I, application no. 15, has been published in F. And the activity of the gene product thereof is the activity of citrate synthase.

Accordingly, in one embodiment, the process of the present invention for producing succinate in a non-human organism, like a microorganism or a plant or a part thereof, comprises increasing or generating the activity of a gene product with the activity of a gene product conferring the activity of “citrate synthase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said At2g44350, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At2g44350, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At2g44350, or a functional equivalent or a homolog thereof as depicted in column 8 of Table II, application no. 15, preferably a homolog or functional equivalent as depicted in column 8 of Table II B, application no. 15, and being depicted in the same respective line as said At2g44350, and preferably the activity is increased plastidic, whereby the respective line disclose in column 7 the fine chemical succinate.

Accordingly, in one embodiment, the molecule which activity is to be increased in the process of the invention is the gene product with an activity as a “citrate synthase”, preferably it is the molecule of section (a) or (b) of this paragraph.

In particular, it was observed that in plants, especially in Arabidopsis thaliana, increasing or generating the activity of a gene product plastidic with the activity of a “citrate synthase”, preferably being encoded by a gene comprising the nucleic acid sequence SEQ ID NO. 120810, preferably the coding region thereof, conferred the production of or the increase in succinate compared with the wild type control.

The nucleic acid sequence of At3g04050 from Arabidopsis thaliana, e.g. as shown in the respective line in column 5 of Table I, application no. 15, has been published in F. And the activity of the gene product thereof is the activity of pyruvate kinase.

Accordingly, in one embodiment, the process of the present invention for producing citramalate in a non-human organism, like a microorganism or a plant or a part thereof, comprises increasing or generating the activity of a gene product with the activity of a gene product conferring the activity of “pyruvate kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said At3g04050, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At3g04050, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At3g04050, or a functional equivalent or a homolog thereof as depicted in column 8 of Table II, application no. 15, preferably a homolog or functional equivalent as depicted in column 8 of Table II B, application no. 15, and being depicted in the same respective line as said At3g04050, and preferably the activity is increased plastidic, whereby the respective line disclose in column 7 the fine chemical citramalate.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 21497, preferably the coding region thereof, conferred the production of or the increase in citramalate compared with the wild type control.

The nucleic acid sequence of At3g04050 from Arabidopsis thaliana, e.g. as shown in the respective line in column 5 of Table I, application no. 15, has been published in F. And the activity of the gene product thereof is the activity of pyruvate kinase.

Accordingly, in one embodiment, the process of the present invention for producing succinate in a non-human organism, like a microorganism or a plant or a part thereof, comprises increasing or generating the activity of a gene product with the activity of a gene product conferring the activity of “pyruvate kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said At3g04050, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At3g04050, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At3g04050, or a functional equivalent or a homolog thereof as depicted in column 8 of Table II, application no. 15, preferably a homolog or functional equivalent as depicted in column 8 of Table II B, application no. 15, and being depicted in the same respective line as said At3g04050, and preferably the activity is increased plastidic, whereby the respective line disclose in column 7 the fine chemical succinate.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 21497, preferably the coding region thereof, conferred the production of or the increase in succinate compared with the wild type control.

The nucleic acid sequence of At3g06270 from Arabidopsis thaliana, e.g. as shown in the respective line in column 5 of Table I, application no. 15, has been published in F. And the activity of the gene product thereof is the activity of protein phosphatase.

Accordingly, in one embodiment, the process of the present invention for producing malate in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said At3g06270, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At3g06270, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At3g06270, or a functional equivalent or a homolog thereof as depicted in column 8 of Table II, application no. 15, preferably a homolog or functional equivalent as depicted in column 8 of Table II B, application no. 15, and being depicted in the same respective line as said At3g06270, and preferably the activity is increased non-targeted, whereby the respective line disclose in column 7 the fine chemical malate.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 22015, preferably the coding region thereof, conferred the production of or the increase in malate compared with the wild type control.

The nucleic acid sequence of At3g06270 from Arabidopsis thaliana, e.g. as shown in the respective line in column 5 of Table I, application no. 15, has been published in F. And the activity of the gene product thereof is the activity of protein phosphatase.

Accordingly, in one embodiment, the process of the present invention for producing succinate in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said At3g06270, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At3g06270, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At3g06270, or a functional equivalent or a homolog thereof as depicted in column 8 of Table II, application no. 15, preferably a homolog or functional equivalent as depicted in column 8 of Table II B, application no. 15, and being depicted in the same respective line as said At3g06270, and preferably the activity is increased non-targeted, whereby the respective line disclose in column 7 the fine chemical succinate.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 22015, preferably the coding region thereof, conferred the production of or the increase in succinate compared with the wild type control.

The nucleic acid sequence of At3g08710 from Arabidopsis thaliana, e.g. as shown in the respective line in column 5 of Table I, application no. 15, has been published in F. And the activity of the gene product thereof is the activity of thioredoxin family protein.

Accordingly, in one embodiment, the process of the present invention for producing fumarate in a non-human organism, like a microorganism or a plant or a part thereof, comprises increasing or generating the activity of a gene product with the activity of a gene product conferring the activity of “thioredoxin family protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said At3g08710, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At3g08710, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At3g08710, or a functional equivalent or a homolog thereof as depicted in column 8 of Table II, application no. 15, preferably a homolog or functional equivalent as depicted in column 8 of Table II B, application no. 15, and being depicted in the same respective line as said At3g08710, and preferably the activity is increased non-targeted, whereby the respective line disclose in column 7 the fine chemical fumarate.

Accordingly, in one embodiment, the molecule which activity is to be increased in the process of the invention is the gene product with an activity as a “thioredoxin family protein”, preferably it is the molecule of section (a) or (b) of this paragraph.

In particular, it was observed that in plants, especially in Arabidopsis thaliana, increasing or generating the activity of a gene product non-targeted with the activity of a “thioredoxin family protein”, preferably being encoded by a gene comprising the nucleic acid sequence SEQ ID NO. 22249, preferably the coding region thereof, conferred the production of or the increase in fumarate compared with the wild type control.

The nucleic acid sequence of At3g08710 from Arabidopsis thaliana, e.g. as shown in the respective line in column 5 of Table I, application no. 15, has been published in F. And the activity of the gene product thereof is the activity of thioredoxin family protein.

Accordingly, in one embodiment, the process of the present invention for producing malate in a non-human organism, like a microorganism or a plant or a part thereof, comprises increasing or generating the activity of a gene product with the activity of a gene product conferring the activity of “thioredoxin family protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said At3g08710, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At3g08710, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At3g08710, or a functional equivalent or a homolog thereof as depicted in column 8 of Table II, application no. 15, preferably a homolog or functional equivalent as depicted in column 8 of Table II B, application no. 15, and being depicted in the same respective line as said At3g08710, and preferably the activity is increased non-targeted, whereby the respective line disclose in column 7 the fine chemical malate.

Accordingly, in one embodiment, the molecule which activity is to be increased in the process of the invention is the gene product with an activity as a “thioredoxin family protein”, preferably it is the molecule of section (a) or (b) of this paragraph.

In particular, it was observed that in plants, especially in Arabidopsis thaliana, increasing or generating the activity of a gene product non-targeted with the activity of a “thioredoxin family protein”, preferably being encoded by a gene comprising the nucleic acid sequence SEQ ID NO. 22249, preferably the coding region thereof, conferred the production of or the increase in malate compared with the wild type control.

The nucleic acid sequence of At3g15360 from Arabidopsis thaliana, e.g. as shown in the respective line in column 5 of Table I, application no. 15, has been published in F. And the activity of the gene product thereof is the activity of thioredoxin.

Accordingly, in one embodiment, the process of the present invention for producing glycerate in a non-human organism, like a microorganism or a plant or a part thereof, comprises increasing or generating the activity of a gene product with the activity of a gene product conferring the activity of “thioredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glycerate), application no. 15, preferably the coding region thereof, or a homolog or a fragment thereof, and being depicted in the same respective line as said At3g15360, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At3g15360, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At3g15360, or a functional equivalent or a homolog thereof as depicted in column 8 of Table II, application no. 15, preferably a homolog or functional equivalent as depicted in column 8 of Table II B, application no. 15, and being depicted in the same respective line as said At3g15360, and preferably the activity is increased non-targeted, whereby the respective line disclose in column 7 the fine chemical glycerate.

Accordingly, in one embodiment, the molecule which activity is to be increased in the process of the invention is the gene product with an activity as a “thioredoxin”, preferably it is the molecule of section (a) or (b) of this paragraph.

In particular, it was observed that in plants, especially in Arabidopsis thaliana, increasing or generating the activity of a gene product non-targeted with the activity of a “thioredoxin”, preferably being encoded by a gene comprising the nucleic acid sequence SEQ ID NO. 130491, preferably the coding region thereof, conferred the production of or the increase in glycerate compared with the wild type control.

The nucleic acid sequence of At3g61830 from Arabidopsis thaliana, e.g. as shown in the respective line in column 5 of Table I, application no. 15, has been published in F. And the activity of the gene product thereof is the activity of auxin response factor.

Accordingly, in one embodiment, the process of the present invention for producing fumarate in a non-human organism, like a microorganism or a plant or a part thereof, comprises increasing or generating the activity of a gene product with the activity of a gene product conferring the activity of “auxin response factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said At3g61830, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At3g61830, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At3g61830, or a functional equivalent or a homolog thereof as depicted in column 8 of Table II, application no. 15, preferably a homolog or functional equivalent as depicted in column 8 of Table II B, application no. 15, and being depicted in the same respective line as said At3g61830, and preferably the activity is increased non-targeted, whereby the respective line disclose in column 7 the fine chemical fumarate.

Accordingly, in one embodiment, the molecule which activity is to be increased in the process of the invention is the gene product with an activity as a “auxin response factor”, preferably it is the molecule of section (a) or (b) of this paragraph.

In particular, it was observed that in plants, especially in Arabidopsis thaliana, increasing or generating the activity of a gene product non-targeted with the activity of a “auxin response factor”, preferably being encoded by a gene comprising the nucleic acid sequence SEQ ID NO. 2367, preferably the coding region thereof, conferred the production of or the increase in fumarate compared with the wild type control.

The nucleic acid sequence of At3g61830 from Arabidopsis thaliana, e.g. as shown in the respective line in column 5 of Table I, application no. 15, has been published in F. And the activity of the gene product thereof is the activity of auxin response factor.

Accordingly, in one embodiment, the process of the present invention for producing malate in a non-human organism, like a microorganism or a plant or a part thereof, comprises increasing or generating the activity of a gene product with the activity of a gene product conferring the activity of “auxin response factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said At3g61830, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At3g61830, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At3g61830, or a functional equivalent or a homolog thereof as depicted in column 8 of Table II, application no. 15, preferably a homolog or functional equivalent as depicted in column 8 of Table II B, application no. 15, and being depicted in the same respective line as said At3g61830, and preferably the activity is increased non-targeted, whereby the respective line disclose in column 7 the fine chemical malate.

Accordingly, in one embodiment, the molecule which activity is to be increased in the process of the invention is the gene product with an activity as a “auxin response factor”, preferably it is the molecule of section (a) or (b) of this paragraph.

In particular, it was observed that in plants, especially in Arabidopsis thaliana, increasing or generating the activity of a gene product non-targeted with the activity of a “auxin response factor”, preferably being encoded by a gene comprising the nucleic acid sequence SEQ ID NO. 2367, preferably the coding region thereof, conferred the production of or the increase in malate compared with the wild type control.

The nucleic acid sequence of At3g62930 from Arabidopsis thaliana, e.g. as shown in the respective line in column 5 of Table I, application no. 15, has been published in F. And the activity of the gene product thereof is the activity of monthiol glutaredoxin.

Accordingly, in one embodiment, the process of the present invention for producing citramalate in a non-human organism, like a microorganism or a plant or a part thereof, comprises increasing or generating the activity of a gene product with the activity of a gene product conferring the activity of “monthiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said At3g62930, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At3g62930, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At3g62930, or a functional equivalent or a homolog thereof as depicted in column 8 of Table II, application no. 15, preferably a homolog or functional equivalent as depicted in column 8 of Table II B, application no. 15, and being depicted in the same respective line as said At3g62930, and preferably the activity is increased non-targeted, whereby the respective line disclose in column 7 the fine chemical citramalate.

Accordingly, in one embodiment, the molecule which activity is to be increased in the process of the invention is the gene product with an activity as a “monthiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monthiol glutaredoxin”, preferably being encoded by a gene comprising the nucleic acid sequence SEQ ID NO. 2573, preferably the coding region thereof, conferred the production of or the increase in citramalate compared with the wild type control.

The nucleic acid sequence of At3g62930 from Arabidopsis thaliana, e.g. as shown in the respective line in column 5 of Table I, application no. 15, has been published in F. And the activity of the gene product thereof is the activity of monthiol glutaredoxin.

Accordingly, in one embodiment, the process of the present invention for producing fumarate in a non-human organism, like a microorganism or a plant or a part thereof, comprises increasing or generating the activity of a gene product with the activity of a gene product conferring the activity of “monthiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said At3g62930, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At3g62930, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At3g62930, or a functional equivalent or a homolog thereof as depicted in column 8 of Table II, application no. 15, preferably a homolog or functional equivalent as depicted in column 8 of Table II B, application no. 15, and being depicted in the same respective line as said At3g62930, and preferably the activity is increased non-targeted, whereby the respective line disclose in column 7 the fine chemical fumarate.

Accordingly, in one embodiment, the molecule which activity is to be increased in the process of the invention is the gene product with an activity as a “monthiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monthiol glutaredoxin”, preferably being encoded by a gene comprising the nucleic acid sequence SEQ ID NO. 2573, preferably the coding region thereof, conferred the production of or the increase in fumarate compared with the wild type control.

The nucleic acid sequence of At3g62930 from Arabidopsis thaliana, e.g. as shown in the respective line in column 5 of Table I, application no. 15, has been published in F. And the activity of the gene product thereof is the activity of monthiol glutaredoxin.

Accordingly, in one embodiment, the process of the present invention for producing glycerate in a non-human organism, like a microorganism or a plant or a part thereof, comprises increasing or generating the activity of a gene product with the activity of a gene product conferring the activity of “monthiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glycerate), application no. 15, preferably the coding region thereof, or a homolog or a fragment thereof, and being depicted in the same respective line as said At3g62930, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At3g62930, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At3g62930, or a functional equivalent or a homolog thereof as depicted in column 8 of Table II, application no. 15, preferably a homolog or functional equivalent as depicted in column 8 of Table II B, application no. 15, and being depicted in the same respective line as said At3g62930, and preferably the activity is increased non-targeted, whereby the respective line disclose in column 7 the fine chemical glycerate.

Accordingly, in one embodiment, the molecule which activity is to be increased in the process of the invention is the gene product with an activity as a “monthiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monthiol glutaredoxin”, preferably being encoded by a gene comprising the nucleic acid sequence SEQ ID NO. 2573, preferably the coding region thereof, conferred the production of or the increase in glycerate compared with the wild type control.

The nucleic acid sequence of At3g62930 from Arabidopsis thaliana, e.g. as shown in the respective line in column 5 of Table I, application no. 15, has been published in F. And the activity of the gene product thereof is the activity of monthiol glutaredoxin.

Accordingly, in one embodiment, the process of the present invention for producing malate in a non-human organism, like a microorganism or a plant or a part thereof, comprises increasing or generating the activity of a gene product with the activity of a gene product conferring the activity of “monthiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said At3g62930, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At3g62930, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At3g62930, or a functional equivalent or a homolog thereof as depicted in column 8 of Table II, application no. 15, preferably a homolog or functional equivalent as depicted in column 8 of Table II B, application no. 15, and being depicted in the same respective line as said At3g62930, and preferably the activity is increased non-targeted, whereby the respective line disclose in column 7 the fine chemical malate.

Accordingly, in one embodiment, the molecule which activity is to be increased in the process of the invention is the gene product with an activity as a “monthiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monthiol glutaredoxin”, preferably being encoded by a gene comprising the nucleic acid sequence SEQ ID NO. 2573, preferably the coding region thereof, conferred the production of or the increase in malate compared with the wild type control.

The nucleic acid sequence of At3g62930 from Arabidopsis thaliana, e.g. as shown in the respective line in column 5 of Table I, application no. 15, has been published in F. And the activity of the gene product thereof is the activity of monthiol glutaredoxin.

Accordingly, in one embodiment, the process of the present invention for producing succinate in a non-human organism, like a microorganism or a plant or a part thereof, comprises increasing or generating the activity of a gene product with the activity of a gene product conferring the activity of “monthiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said At3g62930, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At3g62930, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At3g62930, or a functional equivalent or a homolog thereof as depicted in column 8 of Table II, application no. 15, preferably a homolog or functional equivalent as depicted in column 8 of Table II B, application no. 15, and being depicted in the same respective line as said At3g62930, and preferably the activity is increased non-targeted, whereby the respective line disclose in column 7 the fine chemical succinate.

Accordingly, in one embodiment, the molecule which activity is to be increased in the process of the invention is the gene product with an activity as a “monthiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monthiol glutaredoxin”, preferably being encoded by a gene comprising the nucleic acid sequence SEQ ID NO. 2573, preferably the coding region thereof, conferred the production of or the increase in succinate compared with the wild type control.

The nucleic acid sequence of At3g62930 from Arabidopsis thaliana, e.g. as shown in the respective line in column 5 of Table I, application no. 15, has been published in F. And the activity of the gene product thereof is the activity of monthiol glutaredoxin.

Accordingly, in one embodiment, the process of the present invention for producing vitamin c and/or threonic acid in a non-human organism, like a microorganism or a plant or a part thereof, comprises increasing or generating the activity of a gene product with the activity of a gene product conferring the activity of “monthiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical vitamin c and/or threonic acid), application no. 15, preferably the coding region thereof, or a homolog or a fragment thereof, and being depicted in the same respective line as said At3g62930, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At3g62930, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At3g62930, or a functional equivalent or a homolog thereof as depicted in column 8 of Table II, application no. 15, preferably a homolog or functional equivalent as depicted in column 8 of Table II B, application no. 15, and being depicted in the same respective line as said At3g62930, and preferably the activity is increased non-targeted, whereby the respective line disclose in column 7 the fine chemical vitamin c and/or threonic acid.

Accordingly, in one embodiment, the molecule which activity is to be increased in the process of the invention is the gene product with an activity as a “monthiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monthiol glutaredoxin”, preferably being encoded by a gene comprising the nucleic acid sequence SEQ ID NO. 2573, preferably the coding region thereof, conferred the production of or the increase in vitamin c and/or threonic acid compared with the wild type control.

The nucleic acid sequence of At3g62950 from Arabidopsis thaliana, e.g. as shown in the respective line in column 5 of Table I, application no. 15, has been published in F. And the activity of the gene product thereof is the activity of glutaredoxin.

Accordingly, in one embodiment, the process of the present invention for producing citramalate in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said At3g62950, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At3g62950, or a functional equivalent or a homolog thereof as depicted in column 8 of Table II, application no. 15, preferably a homolog or functional equivalent as depicted in column 8 of Table II B, application no. 15, and being depicted in the same respective line as said At3g62950, and preferably the activity is increased non-targeted, whereby the respective line disclose in column 7 the fine chemical citramalate.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in citramalate compared with the wild type control.

The nucleic acid sequence of At3g62950 from Arabidopsis thaliana, e.g. as shown in the respective line in column 5 of Table I, application no. 15, has been published in F. And the activity of the gene product thereof is the activity of glutaredoxin.

Accordingly, in one embodiment, the process of the present invention for producing fumarate in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said At3g62950, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At3g62950, or a functional equivalent or a homolog thereof as depicted in column 8 of Table II, application no. 15, preferably a homolog or functional equivalent as depicted in column 8 of Table II B, application no. 15, and being depicted in the same respective line as said At3g62950, and preferably the activity is increased non-targeted, whereby the respective line disclose in column 7 the fine chemical fumarate.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in fumarate compared with the wild type control.

The nucleic acid sequence of At3g62950 from Arabidopsis thaliana, e.g. as shown in the respective line in column 5 of Table I, application no. 15, has been published in F. And the activity of the gene product thereof is the activity of glutaredoxin.

Accordingly, in one embodiment, the process of the present invention for producing glycerate in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glycerate), application no. 15, preferably the coding region thereof, or a homolog or a fragment thereof, and being depicted in the same respective line as said At3g62950, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At3g62950, or a functional equivalent or a homolog thereof as depicted in column 8 of Table II, application no. 15, preferably a homolog or functional equivalent as depicted in column 8 of Table II B, application no. 15, and being depicted in the same respective line as said At3g62950, and preferably the activity is increased non-targeted, whereby the respective line disclose in column 7 the fine chemical glycerate.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in glycerate compared with the wild type control.

The nucleic acid sequence of At3g62950 from Arabidopsis thaliana, e.g. as shown in the respective line in column 5 of Table I, application no. 15, has been published in F. And the activity of the gene product thereof is the activity of glutaredoxin.

Accordingly, in one embodiment, the process of the present invention for producing malate in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said At3g62950, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At3g62950, or a functional equivalent or a homolog thereof as depicted in column 8 of Table II, application no. 15, preferably a homolog or functional equivalent as depicted in column 8 of Table II B, application no. 15, and being depicted in the same respective line as said At3g62950, and preferably the activity is increased non-targeted, whereby the respective line disclose in column 7 the fine chemical malate.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in malate compared with the wild type control.

The nucleic acid sequence of At3g62950 from Arabidopsis thaliana, e.g. as shown in the respective line in column 5 of Table I, application no. 15, has been published in F. And the activity of the gene product thereof is the activity of glutaredoxin.

Accordingly, in one embodiment, the process of the present invention for producing succinate in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said At3g62950, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At3g62950, or a functional equivalent or a homolog thereof as depicted in column 8 of Table II, application no. 15, preferably a homolog or functional equivalent as depicted in column 8 of Table II B, application no. 15, and being depicted in the same respective line as said At3g62950, and preferably the activity is increased non-targeted, whereby the respective line disclose in column 7 the fine chemical succinate.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in succinate compared with the wild type control.

The nucleic acid sequence of At4g03520 from Arabidopsis thaliana, e.g. as shown in the respective line in column 5 of Table I, application no. 15, has been published in F. And the activity of the gene product thereof is the activity of thioredoxin.

Accordingly, in one embodiment, the process of the present invention for producing glycerate in a non-human organism, like a microorganism or a plant or a part thereof, comprises increasing or generating the activity of a gene product with the activity of a gene product conferring the activity of “thioredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glycerate), application no. 15, preferably the coding region thereof, or a homolog or a fragment thereof, and being depicted in the same respective line as said At4g03520, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At4g03520, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At4g03520, or a functional equivalent or a homolog thereof as depicted in column 8 of Table II, application no. 15, preferably a homolog or functional equivalent as depicted in column 8 of Table II B, application no. 15, and being depicted in the same respective line as said At4g03520, and preferably the activity is increased non-targeted, whereby the respective line disclose in column 7 the fine chemical glycerate.

Accordingly, in one embodiment, the molecule which activity is to be increased in the process of the invention is the gene product with an activity as a “thioredoxin”, preferably it is the molecule of section (a) or (b) of this paragraph.

In particular, it was observed that in plants, especially in Arabidopsis thaliana, increasing or generating the activity of a gene product non-targeted with the activity of a “thioredoxin”, preferably being encoded by a gene comprising the nucleic acid sequence SEQ ID NO. 133541, preferably the coding region thereof, conferred the production of or the increase in glycerate compared with the wild type control.

The nucleic acid sequence of At4g15660 from Arabidopsis thaliana, e.g. as shown in the respective line in column 5 of Table I, application no. 15, has been published in F. And the activity of the gene product thereof is the activity of monothiol glutaredoxin.

Accordingly, in one embodiment, the process of the present invention for producing citramalate in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said At4g15660, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At4g15660, or a functional equivalent or a homolog thereof as depicted in column 8 of Table II, application no. 15, preferably a homolog or functional equivalent as depicted in column 8 of Table II B, application no. 15, and being depicted in the same respective line as said At4g15660, and preferably the activity is increased non-targeted, whereby the respective line disclose in column 7 the fine chemical citramalate.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in citramalate compared with the wild type control.

The nucleic acid sequence of At4g15660 from Arabidopsis thaliana, e.g. as shown in the respective line in column 5 of Table I, application no. 15, has been published in F. And the activity of the gene product thereof is the activity of monothiol glutaredoxin.

Accordingly, in one embodiment, the process of the present invention for producing fumarate in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said At4g15660, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At4g15660, or a functional equivalent or a homolog thereof as depicted in column 8 of Table II, application no. 15, preferably a homolog or functional equivalent as depicted in column 8 of Table II B, application no. 15, and being depicted in the same respective line as said At4g15660, and preferably the activity is increased non-targeted, whereby the respective line disclose in column 7 the fine chemical fumarate.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in fumarate compared with the wild type control.

The nucleic acid sequence of At4g15660 from Arabidopsis thaliana, e.g. as shown in the respective line in column 5 of Table I, application no. 15, has been published in F. And the activity of the gene product thereof is the activity of monothiol glutaredoxin.

Accordingly, in one embodiment, the process of the present invention for producing glycerate in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glycerate), application no. 15, preferably the coding region thereof, or a homolog or a fragment thereof, and being depicted in the same respective line as said At4g15660, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At4g15660, or a functional equivalent or a homolog thereof as depicted in column 8 of Table II, application no. 15, preferably a homolog or functional equivalent as depicted in column 8 of Table II B, application no. 15, and being depicted in the same respective line as said At4g15660, and preferably the activity is increased non-targeted, whereby the respective line disclose in column 7 the fine chemical glycerate.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in glycerate compared with the wild type control.

The nucleic acid sequence of At4g15660 from Arabidopsis thaliana, e.g. as shown in the respective line in column 5 of Table I, application no. 15, has been published in F. And the activity of the gene product thereof is the activity of monothiol glutaredoxin.

Accordingly, in one embodiment, the process of the present invention for producing malate in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said At4g15660, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At4g15660, or a functional equivalent or a homolog thereof as depicted in column 8 of Table II, application no. 15, preferably a homolog or functional equivalent as depicted in column 8 of Table II B, application no. 15, and being depicted in the same respective line as said At4g15660, and preferably the activity is increased non-targeted, whereby the respective line disclose in column 7 the fine chemical malate.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in malate compared with the wild type control.

The nucleic acid sequence of At4g15660 from Arabidopsis thaliana, e.g. as shown in the respective line in column 5 of Table I, application no. 15, has been published in F. And the activity of the gene product thereof is the activity of monothiol glutaredoxin.

Accordingly, in one embodiment, the process of the present invention for producing succinate in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said At4g15660, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At4g15660, or a functional equivalent or a homolog thereof as depicted in column 8 of Table II, application no. 15, preferably a homolog or functional equivalent as depicted in column 8 of Table II B, application no. 15, and being depicted in the same respective line as said At4g15660, and preferably the activity is increased non-targeted, whereby the respective line disclose in column 7 the fine chemical succinate.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in succinate compared with the wild type control.

The nucleic acid sequence of At4g15660 from Arabidopsis thaliana, e.g. as shown in the respective line in column 5 of Table I, application no. 15, has been published in F. And the activity of the gene product thereof is the activity of monothiol glutaredoxin.

Accordingly, in one embodiment, the process of the present invention for producing vitamin c and/or threonic acid in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical vitamin c and/or threonic acid), application no. 15, preferably the coding region thereof, or a homolog or a fragment thereof, and being depicted in the same respective line as said At4g15660, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At4g15660, or a functional equivalent or a homolog thereof as depicted in column 8 of Table II, application no. 15, preferably a homolog or functional equivalent as depicted in column 8 of Table II B, application no. 15, and being depicted in the same respective line as said At4g15660, and preferably the activity is increased non-targeted, whereby the respective line disclose in column 7 the fine chemical vitamin c and/or threonic acid.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in vitamin c and/or threonic acid compared with the wild type control.

The nucleic acid sequence of At4g15670 from Arabidopsis thaliana, e.g. as shown in the respective line in column 5 of Table I, application no. 15, has been published in F. And the activity of the gene product thereof is the activity of monothiol glutaredoxin.

Accordingly, in one embodiment, the process of the present invention for producing fumarate in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said At4g15670, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At4g15670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At4g15670, or a functional equivalent or a homolog thereof as depicted in column 8 of Table II, application no. 15, preferably a homolog or functional equivalent as depicted in column 8 of Table II B, application no. 15, and being depicted in the same respective line as said At4g15670, and preferably the activity is increased non-targeted, whereby the respective line disclose in column 7 the fine chemical fumarate.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3279, preferably the coding region thereof, conferred the production of or the increase in fumarate compared with the wild type control.

The nucleic acid sequence of At4g15670 from Arabidopsis thaliana, e.g. as shown in the respective line in column 5 of Table I, application no. 15, has been published in F. And the activity of the gene product thereof is the activity of monothiol glutaredoxin.

Accordingly, in one embodiment, the process of the present invention for producing glycerate in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glycerate), application no. 15, preferably the coding region thereof, or a homolog or a fragment thereof, and being depicted in the same respective line as said At4g15670, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At4g15670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At4g15670, or a functional equivalent or a homolog thereof as depicted in column 8 of Table II, application no. 15, preferably a homolog or functional equivalent as depicted in column 8 of Table II B, application no. 15, and being depicted in the same respective line as said At4g15670, and preferably the activity is increased non-targeted, whereby the respective line disclose in column 7 the fine chemical glycerate.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3279, preferably the coding region thereof, conferred the production of or the increase in glycerate compared with the wild type control.

The nucleic acid sequence of At4g15670 from Arabidopsis thaliana, e.g. as shown in the respective line in column 5 of Table I, application no. 15, has been published in F. And the activity of the gene product thereof is the activity of monothiol glutaredoxin.

Accordingly, in one embodiment, the process of the present invention for producing malate in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said At4g15670, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At4g15670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At4g15670, or a functional equivalent or a homolog thereof as depicted in column 8 of Table II, application no. 15, preferably a homolog or functional equivalent as depicted in column 8 of Table II B, application no. 15, and being depicted in the same respective line as said At4g15670, and preferably the activity is increased non-targeted, whereby the respective line disclose in column 7 the fine chemical malate.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3279, preferably the coding region thereof, conferred the production of or the increase in malate compared with the wild type control.

The nucleic acid sequence of At4g15670 from Arabidopsis thaliana, e.g. as shown in the respective line in column 5 of Table I, application no. 15, has been published in F. And the activity of the gene product thereof is the activity of monothiol glutaredoxin.

Accordingly, in one embodiment, the process of the present invention for producing succinate in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said At4g15670, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At4g15670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At4g15670, or a functional equivalent or a homolog thereof as depicted in column 8 of Table II, application no. 15, preferably a homolog or functional equivalent as depicted in column 8 of Table II B, application no. 15, and being depicted in the same respective line as said At4g15670, and preferably the activity is increased non-targeted, whereby the respective line disclose in column 7 the fine chemical succinate.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3279, preferably the coding region thereof, conferred the production of or the increase in succinate compared with the wild type control.

The nucleic acid sequence of At4g15690 from Arabidopsis thaliana, e.g. as shown in the respective line in column 5 of Table I, application no. 15, has been published in F. And the activity of the gene product thereof is the activity of monothiol glutaredoxin.

Accordingly, in one embodiment, the process of the present invention for producing citramalate in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said At4g15690, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At4g15690, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At4g15690, or a functional equivalent or a homolog thereof as depicted in column 8 of Table II, application no. 15, preferably a homolog or functional equivalent as depicted in column 8 of Table II B, application no. 15, and being depicted in the same respective line as said At4g15690, and preferably the activity is increased non-targeted, whereby the respective line disclose in column 7 the fine chemical citramalate.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23844, preferably the coding region thereof, conferred the production of or the increase in citramalate compared with the wild type control.

The nucleic acid sequence of At4g15690 from Arabidopsis thaliana, e.g. as shown in the respective line in column 5 of Table I, application no. 15, has been published in F. And the activity of the gene product thereof is the activity of monothiol glutaredoxin.

Accordingly, in one embodiment, the process of the present invention for producing fumarate in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said At4g15690, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At4g15690, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At4g15690, or a functional equivalent or a homolog thereof as depicted in column 8 of Table II, application no. 15, preferably a homolog or functional equivalent as depicted in column 8 of Table II B, application no. 15, and being depicted in the same respective line as said At4g15690, and preferably the activity is increased non-targeted, whereby the respective line disclose in column 7 the fine chemical fumarate.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23844, preferably the coding region thereof, conferred the production of or the increase in fumarate compared with the wild type control.

The nucleic acid sequence of At4g15690 from Arabidopsis thaliana, e.g. as shown in the respective line in column 5 of Table I, application no. 15, has been published in F. And the activity of the gene product thereof is the activity of monothiol glutaredoxin.

Accordingly, in one embodiment, the process of the present invention for producing malate in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said At4g15690, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At4g15690, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At4g15690, or a functional equivalent or a homolog thereof as depicted in column 8 of Table II, application no. 15, preferably a homolog or functional equivalent as depicted in column 8 of Table II B, application no. 15, and being depicted in the same respective line as said At4g15690, and preferably the activity is increased non-targeted, whereby the respective line disclose in column 7 the fine chemical malate.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23844, preferably the coding region thereof, conferred the production of or the increase in malate compared with the wild type control.

The nucleic acid sequence of At4g15690 from Arabidopsis thaliana, e.g. as shown in the respective line in column 5 of Table I, application no. 15, has been published in F. And the activity of the gene product thereof is the activity of monothiol glutaredoxin.

Accordingly, in one embodiment, the process of the present invention for producing succinate in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said At4g15690, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At4g15690, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At4g15690, or a functional equivalent or a homolog thereof as depicted in column 8 of Table II, application no. 15, preferably a homolog or functional equivalent as depicted in column 8 of Table II B, application no. 15, and being depicted in the same respective line as said At4g15690, and preferably the activity is increased non-targeted, whereby the respective line disclose in column 7 the fine chemical succinate.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23844, preferably the coding region thereof, conferred the production of or the increase in succinate compared with the wild type control.

The nucleic acid sequence of At4g15690 from Arabidopsis thaliana, e.g. as shown in the respective line in column 5 of Table I, application no. 15, has been published in F. And the activity of the gene product thereof is the activity of monothiol glutaredoxin.

Accordingly, in one embodiment, the process of the present invention for producing vitamin c and/or threonic acid in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical vitamin c and/or threonic acid), application no. 15, preferably the coding region thereof, or a homolog or a fragment thereof, and being depicted in the same respective line as said At4g15690, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At4g15690, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At4g15690, or a functional equivalent or a homolog thereof as depicted in column 8 of Table II, application no. 15, preferably a homolog or functional equivalent as depicted in column 8 of Table II B, application no. 15, and being depicted in the same respective line as said At4g15690, and preferably the activity is increased non-targeted, whereby the respective line disclose in column 7 the fine chemical vitamin c and/or threonic acid.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23844, preferably the coding region thereof, conferred the production of or the increase in vitamin c and/or threonic acid compared with the wild type control.

The nucleic acid sequence of At4g15700 from Arabidopsis thaliana, e.g. as shown in the respective line in column 5 of Table I, application no. 15, has been published in F. And the activity of the gene product thereof is the activity of monothiol glutaredoxin.

Accordingly, in one embodiment, the process of the present invention for producing glycerate in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glycerate), application no. 15, preferably the coding region thereof, or a homolog or a fragment thereof, and being depicted in the same respective line as said At4g15700, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At4g15700, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At4g15700, or a functional equivalent or a homolog thereof as depicted in column 8 of Table II, application no. 15, preferably a homolog or functional equivalent as depicted in column 8 of Table II B, application no. 15, and being depicted in the same respective line as said At4g15700, and preferably the activity is increased non-targeted, whereby the respective line disclose in column 7 the fine chemical glycerate.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3654, preferably the coding region thereof, conferred the production of or the increase in glycerate compared with the wild type control.

The nucleic acid sequence of At4g26080 from Arabidopsis thaliana, e.g. as shown in the respective line in column 5 of Table I, application no. 15, has been published in F. And the activity of the gene product thereof is the activity of protein phosphatase.

Accordingly, in one embodiment, the process of the present invention for producing citramalate in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said At4g26080, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At4g26080, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At4g26080, or a functional equivalent or a homolog thereof as depicted in column 8 of Table II, application no. 15, preferably a homolog or functional equivalent as depicted in column 8 of Table II B, application no. 15, and being depicted in the same respective line as said At4g26080, and preferably the activity is increased non-targeted, whereby the respective line disclose in column 7 the fine chemical citramalate.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 68849, preferably the coding region thereof, conferred the production of or the increase in citramalate compared with the wild type control.

The nucleic acid sequence of At4g26080 from Arabidopsis thaliana, e.g. as shown in the respective line in column 5 of Table I, application no. 15, has been published in F. And the activity of the gene product thereof is the activity of protein phosphatase.

Accordingly, in one embodiment, the process of the present invention for producing succinate in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said At4g26080, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At4g26080, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At4g26080, or a functional equivalent or a homolog thereof as depicted in column 8 of Table II, application no. 15, preferably a homolog or functional equivalent as depicted in column 8 of Table II B, application no. 15, and being depicted in the same respective line as said At4g26080, and preferably the activity is increased non-targeted, whereby the respective line disclose in column 7 the fine chemical succinate.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 68849, preferably the coding region thereof, conferred the production of or the increase in succinate compared with the wild type control.

The nucleic acid sequence of At4g33040 from Arabidopsis thaliana, e.g. as shown in the respective line in column 5 of Table I, application no. 15, has been published in F. And the activity of the gene product thereof is the activity of glutaredoxin.

Accordingly, in one embodiment, the process of the present invention for producing citramalate in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said At4g33040, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At4g33040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At4g33040, or a functional equivalent or a homolog thereof as depicted in column 8 of Table II, application no. 15, preferably a homolog or functional equivalent as depicted in column 8 of Table II B, application no. 15, and being depicted in the same respective line as said At4g33040, and preferably the activity is increased non-targeted, whereby the respective line disclose in column 7 the fine chemical citramalate.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 4102, preferably the coding region thereof, conferred the production of or the increase in citramalate compared with the wild type control.

The nucleic acid sequence of At4g33040 from Arabidopsis thaliana, e.g. as shown in the respective line in column 5 of Table I, application no. 15, has been published in F. And the activity of the gene product thereof is the activity of glutaredoxin.

Accordingly, in one embodiment, the process of the present invention for producing fumarate in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said At4g33040, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At4g33040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At4g33040, or a functional equivalent or a homolog thereof as depicted in column 8 of Table II, application no. 15, preferably a homolog or functional equivalent as depicted in column 8 of Table II B, application no. 15, and being depicted in the same respective line as said At4g33040, and preferably the activity is increased non-targeted, whereby the respective line disclose in column 7 the fine chemical fumarate.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 4102, preferably the coding region thereof, conferred the production of or the increase in fumarate compared with the wild type control.

The nucleic acid sequence of At4g33040 from Arabidopsis thaliana, e.g. as shown in the respective line in column 5 of Table I, application no. 15, has been published in F. And the activity of the gene product thereof is the activity of glutaredoxin.

Accordingly, in one embodiment, the process of the present invention for producing malate in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said At4g33040, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At4g33040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At4g33040, or a functional equivalent or a homolog thereof as depicted in column 8 of Table II, application no. 15, preferably a homolog or functional equivalent as depicted in column 8 of Table II B, application no. 15, and being depicted in the same respective line as said At4g33040, and preferably the activity is increased non-targeted, whereby the respective line disclose in column 7 the fine chemical malate.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 4102, preferably the coding region thereof, conferred the production of or the increase in malate compared with the wild type control.

The nucleic acid sequence of At4g33040 from Arabidopsis thaliana, e.g. as shown in the respective line in column 5 of Table I, application no. 15, has been published in F. And the activity of the gene product thereof is the activity of glutaredoxin.

Accordingly, in one embodiment, the process of the present invention for producing succinate in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said At4g33040, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At4g33040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At4g33040, or a functional equivalent or a homolog thereof as depicted in column 8 of Table II, application no. 15, preferably a homolog or functional equivalent as depicted in column 8 of Table II B, application no. 15, and being depicted in the same respective line as said At4g33040, and preferably the activity is increased non-targeted, whereby the respective line disclose in column 7 the fine chemical succinate.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 4102, preferably the coding region thereof, conferred the production of or the increase in succinate compared with the wild type control.

The nucleic acid sequence of At4g33040 from Arabidopsis thaliana, e.g. as shown in the respective line in column 5 of Table I, application no. 15, has been published in F. And the activity of the gene product thereof is the activity of glutaredoxin.

Accordingly, in one embodiment, the process of the present invention for producing vitamin c and/or threonic acid in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical vitamin c and/or threonic acid), application no. 15, preferably the coding region thereof, or a homolog or a fragment thereof, and being depicted in the same respective line as said At4g33040, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At4g33040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At4g33040, or a functional equivalent or a homolog thereof as depicted in column 8 of Table II, application no. 15, preferably a homolog or functional equivalent as depicted in column 8 of Table II B, application no. 15, and being depicted in the same respective line as said At4g33040, and preferably the activity is increased non-targeted, whereby the respective line disclose in column 7 the fine chemical vitamin c and/or threonic acid.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 4102, preferably the coding region thereof, conferred the production of or the increase in vitamin c and/or threonic acid compared with the wild type control.

The nucleic acid sequence of At4g35310 from Arabidopsis thaliana, e.g. as shown in the respective line in column 5 of Table I, application no. 15, has been published in F. And the activity of the gene product thereof is the activity of calcium-dependent protein kinase.

Accordingly, in one embodiment, the process of the present invention for producing malate in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said At4g35310, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At4g35310, or a functional equivalent or a homolog thereof as depicted in column 8 of Table II, application no. 15, preferably a homolog or functional equivalent as depicted in column 8 of Table II B, application no. 15, and being depicted in the same respective line as said At4g35310, and preferably the activity is increased non-targeted, whereby the respective line disclose in column 7 the fine chemical malate.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in malate compared with the wild type control.

The nucleic acid sequence of At5g03720 from Arabidopsis thaliana, e.g. as shown in the respective line in column 5 of Table I, application no. 15, has been published in F. And the activity of the gene product thereof is the activity of heat shock transcription factor.

Accordingly, in one embodiment, the process of the present invention for producing fumarate in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said At5g03720, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At5g03720, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At5g03720, or a functional equivalent or a homolog thereof as depicted in column 8 of Table II, application no. 15, preferably a homolog or functional equivalent as depicted in column 8 of Table II B, application no. 15, and being depicted in the same respective line as said At5g03720, and preferably the activity is increased non-targeted, whereby the respective line disclose in column 7 the fine chemical fumarate.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 24438, preferably the coding region thereof, conferred the production of or the increase in fumarate compared with the wild type control.

The nucleic acid sequence of At5g03720 from Arabidopsis thaliana, e.g. as shown in the respective line in column 5 of Table I, application no. 15, has been published in F. And the activity of the gene product thereof is the activity of heat shock transcription factor.

Accordingly, in one embodiment, the process of the present invention for producing malate in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said At5g03720, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At5g03720, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At5g03720, or a functional equivalent or a homolog thereof as depicted in column 8 of Table II, application no. 15, preferably a homolog or functional equivalent as depicted in column 8 of Table II B, application no. 15, and being depicted in the same respective line as said At5g03720, and preferably the activity is increased non-targeted, whereby the respective line disclose in column 7 the fine chemical malate.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 24438, preferably the coding region thereof, conferred the production of or the increase in malate compared with the wild type control.

The nucleic acid sequence of At5g03720 from Arabidopsis thaliana, e.g. as shown in the respective line in column 5 of Table I, application no. 15, has been published in F. And the activity of the gene product thereof is the activity of heat shock transcription factor.

Accordingly, in one embodiment, the process of the present invention for producing succinate in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said At5g03720, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At5g03720, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At5g03720, or a functional equivalent or a homolog thereof as depicted in column 8 of Table II, application no. 15, preferably a homolog or functional equivalent as depicted in column 8 of Table II B, application no. 15, and being depicted in the same respective line as said At5g03720, and preferably the activity is increased non-targeted, whereby the respective line disclose in column 7 the fine chemical succinate.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 24438, preferably the coding region thereof, conferred the production of or the increase in succinate compared with the wild type control.

The nucleic acid sequence of At5g07200 from Arabidopsis thaliana, e.g. as shown in the respective line in column 5 of Table I, application no. 15, has been published in F. And the activity of the gene product thereof is the activity of gibberellin 20-oxidase.

Accordingly, in one embodiment, the process of the present invention for producing glycerate in a non-human organism, like a microorganism or a plant or a part thereof, comprises increasing or generating the activity of a gene product with the activity of a gene product conferring the activity of “gibberellin 20-oxidase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glycerate), application no. 15, preferably the coding region thereof, or a homolog or a fragment thereof, and being depicted in the same respective line as said At5g07200, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At5g07200, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At5g07200, or a functional equivalent or a homolog thereof as depicted in column 8 of Table II, application no. 15, preferably a homolog or functional equivalent as depicted in column 8 of Table II B, application no. 15, and being depicted in the same respective line as said At5g07200, and preferably the activity is increased non-targeted, whereby the respective line disclose in column 7 the fine chemical glycerate.

Accordingly, in one embodiment, the molecule which activity is to be increased in the process of the invention is the gene product with an activity as a “gibberellin 20-oxidase”, preferably it is the molecule of section (a) or (b) of this paragraph.

In particular, it was observed that in plants, especially in Arabidopsis thaliana, increasing or generating the activity of a gene product non-targeted with the activity of a “gibberellin 20oxidase”, preferably being encoded by a gene comprising the nucleic acid sequence SEQ ID NO. 24492, preferably the coding region thereof, conferred the production of or the increase in glycerate compared with the wild type control.

The nucleic acid sequence of At5g07200 from Arabidopsis thaliana, e.g. as shown in the respective line in column 5 of Table I, application no. 15, has been published in F. And the activity of the gene product thereof is the activity of gibberellin 20-oxidase.

Accordingly, in one embodiment, the process of the present invention for producing malate in a non-human organism, like a microorganism or a plant or a part thereof, comprises increasing or generating the activity of a gene product with the activity of a gene product conferring the activity of “gibberellin 20-oxidase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said At5g07200, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At5g07200, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At5g07200, or a functional equivalent or a homolog thereof as depicted in column 8 of Table II, application no. 15, preferably a homolog or functional equivalent as depicted in column 8 of Table II B, application no. 15, and being depicted in the same respective line as said At5g07200, and preferably the activity is increased non-targeted, whereby the respective line disclose in column 7 the fine chemical malate.

Accordingly, in one embodiment, the molecule which activity is to be increased in the process of the invention is the gene product with an activity as a “gibberellin 20-oxidase”, preferably it is the molecule of section (a) or (b) of this paragraph.

In particular, it was observed that in plants, especially in Arabidopsis thaliana, increasing or generating the activity of a gene product non-targeted with the activity of a “gibberellin 20-oxidase”, preferably being encoded by a gene comprising the nucleic acid sequence SEQ ID NO. 24492, preferably the coding region thereof, conferred the production of or the increase in malate compared with the wild type control.

The nucleic acid sequence of At5g18600 from Arabidopsis thaliana, e.g. as shown in the respective line in column 5 of Table I, application no. 15, has been published in F. And the activity of the gene product thereof is the activity of monothiol glutaredoxin.

Accordingly, in one embodiment, the process of the present invention for producing succinate in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said At5g18600, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At5g18600, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At5g18600, or a functional equivalent or a homolog thereof as depicted in column 8 of Table II, application no. 15, preferably a homolog or functional equivalent as depicted in column 8 of Table II B, application no. 15, and being depicted in the same respective line as said At5g18600, and preferably the activity is increased non-targeted, whereby the respective line disclose in column 7 the fine chemical succinate.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 4904, preferably the coding region thereof, conferred the production of or the increase in succinate compared with the wild type control.

The nucleic acid sequence of At5g27640 from Arabidopsis thaliana, e.g. as shown in the respective line in column 5 of Table I, application no. 15, has been published in F. And the activity of the gene product thereof is the activity of translation initiation factor subunit.

Accordingly, in one embodiment, the process of the present invention for producing malate in a non-human organism, like a microorganism or a plant or a part thereof, comprises increasing or generating the activity of a gene product with the activity of a gene product conferring the activity of “translation initiation factor subunit”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said At5g27640, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At5g27640, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At5g27640, or a functional equivalent or a homolog thereof as depicted in column 8 of Table II, application no. 15, preferably a homolog or functional equivalent as depicted in column 8 of Table II B, application no. 15, and being depicted in the same respective line as said At5g27640, and preferably the activity is increased non-targeted, whereby the respective line disclose in column 7 the fine chemical malate.

Accordingly, in one embodiment, the molecule which activity is to be increased in the process of the invention is the gene product with an activity as a “translation initiation factor subunit”, preferably it is the molecule of section (a) or (b) of this paragraph.

In particular, it was observed that in plants, especially in Arabidopsis thaliana, increasing or generating the activity of a gene product non-targeted with the activity of a “translation initiation factor subunit”, preferably being encoded by a gene comprising the nucleic acid sequence SEQ ID NO. 25344, preferably the coding region thereof, conferred the production of or the increase in malate compared with the wild type control.

The nucleic acid sequence of At5g39760 from Arabidopsis thaliana, e.g. as shown in the respective line in column 5 of Table I, application no. 15, has been published in F. And the activity of the gene product thereof is the activity of transcription factor.

Accordingly, in one embodiment, the process of the present invention for producing fumarate in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said At5g39760, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At5g39760, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At5g39760, or a functional equivalent or a homolog thereof as depicted in column 8 of Table II, application no. 15, preferably a homolog or functional equivalent as depicted in column 8 of Table II B, application no. 15, and being depicted in the same respective line as said At5g39760, and preferably the activity is increased non-targeted, whereby the respective line disclose in column 7 the fine chemical fumarate.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 25428, preferably the coding region thereof, conferred the production of or the increase in fumarate compared with the wild type control.

The nucleic acid sequence of At5g39760 from Arabidopsis thaliana, e.g. as shown in the respective line in column 5 of Table I, application no. 15, has been published in F. And the activity of the gene product thereof is the activity of transcription factor.

Accordingly, in one embodiment, the process of the present invention for producing malate in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said At5g39760, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At5g39760, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At5g39760, or a functional equivalent or a homolog thereof as depicted in column 8 of Table II, application no. 15, preferably a homolog or functional equivalent as depicted in column 8 of Table II B, application no. 15, and being depicted in the same respective line as said At5g39760, and preferably the activity is increased non-targeted, whereby the respective line disclose in column 7 the fine chemical malate.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 25428, preferably the coding region thereof, conferred the production of or the increase in malate compared with the wild type control.

The nucleic acid sequence of At5g59220 from Arabidopsis thaliana, e.g. as shown in the respective line in column 5 of Table I, application no. 15, has been published in F. And the activity of the gene product thereof is the activity of protein phosphatase.

Accordingly, in one embodiment, the process of the present invention for producing succinate in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said At5g59220, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At5g59220, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At5g59220, or a functional equivalent or a homolog thereof as depicted in column 8 of Table II, application no. 15, preferably a homolog or functional equivalent as depicted in column 8 of Table II B, application no. 15, and being depicted in the same respective line as said At5g59220, and preferably the activity is increased non-targeted, whereby the respective line disclose in column 7 the fine chemical succinate.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 25498, preferably the coding region thereof, conferred the production of or the increase in succinate compared with the wild type control.

The nucleic acid sequence of At5g63680 from Arabidopsis thaliana, e.g. as shown in the respective line in column 5 of Table I, application no. 15, has been published in F. And the activity of the gene product thereof is the activity of pyruvate kinase.

Accordingly, in one embodiment, the process of the present invention for producing succinate in a non-human organism, like a microorganism or a plant or a part thereof, comprises increasing or generating the activity of a gene product with the activity of a gene product conferring the activity of “pyruvate kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said At5g63680, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At5g63680, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At5g63680, or a functional equivalent or a homolog thereof as depicted in column 8 of Table II, application no. 15, preferably a homolog or functional equivalent as depicted in column 8 of Table II B, application no. 15, and being depicted in the same respective line as said At5g63680, and preferably the activity is increased plastidic, whereby the respective line disclose in column 7 the fine chemical succinate.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 70038, preferably the coding region thereof, conferred the production of or the increase in succinate compared with the wild type control.

The nucleic acid sequence of At5g64920 from Arabidopsis thaliana, e.g. as shown in the respective line in column 5 of Table I, application no. 15, has been published in F. And the activity of the gene product thereof is the activity of zinc finger protein.

Accordingly, in one embodiment, the process of the present invention for producing fumarate in a non-human organism, like a microorganism or a plant or a part thereof, comprises increasing or generating the activity of a gene product with the activity of a gene product conferring the activity of “zinc finger protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said At5g64920, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said At5g64920, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said At5g64920, or a functional equivalent or a homolog thereof as depicted in column 8 of Table II, application no. 15, preferably a homolog or functional equivalent as depicted in column 8 of Table II B, application no. 15, and being depicted in the same respective line as said At5g64920, and preferably the activity is increased non-targeted, whereby the respective line disclose in column 7 the fine chemical fumarate.

Accordingly, in one embodiment, the molecule which activity is to be increased in the process of the invention is the gene product with an activity as a “zinc finger protein”, preferably it is the molecule of section (a) or (b) of this paragraph.

In particular, it was observed that in plants, especially in Arabidopsis thaliana, increasing or generating the activity of a gene product non-targeted with the activity of a “zinc finger protein”, preferably being encoded by a gene comprising the nucleic acid sequence SEQ ID NO. 5493, preferably the coding region thereof, conferred the production of or the increase in fumarate compared with the wild type control.

The nucleic acid sequence of AvinDRAFT_{—}0035 from Azotobacter vinelandii, e.g. as shown in the respective line in column 5 of Table I, application no. 15, has been published in D. And the activity of the gene product thereof is the activity of CDP-diacylglycerol-serine O-phosphatidyltransferase.

Accordingly, in one embodiment, the process of the present invention for producing citramalate in a non-human organism, like a microorganism or a plant or a part thereof, comprises increasing or generating the activity of a gene product with the activity of a gene product conferring the activity of “CDP-diacylglycerol-serine O-phosphatidyltransferase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said AvinDRAFT_{—}0035, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}0035, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}0035, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}0035, and preferably the activity is increased non-targeted, whereby the respective line disclose in column 7 the fine chemical citramalate.

Accordingly, in one embodiment, the molecule which activity is to be increased in the process of the invention is the gene product with an activity as a “CDP-diacylglycerol-serine O-phosphatidyltransferase”, preferably it is the molecule of section (a) or (b) of this paragraph.

In particular, it was observed that in plants, especially in Arabidopsis thaliana, increasing or generating the activity of a gene product non-targeted with the activity of a “CDP-diacylglycerol-serine O-phosphatidyltransferase”, preferably being encoded by a gene comprising the nucleic acid sequence SEQ ID NO. 70583, preferably the coding region thereof, conferred the production of or the increase in citramalate compared with the wild type control.

The nucleic acid sequence of AvinDRAFT_{—}0035 from Azotobacter vinelandii, e.g. as shown in the respective line in column 5 of Table I, application no. 15, has been published in D. And the activity of the gene product thereof is the activity of CDP-diacylglycerol-serine O-phosphatidyltransferase.

Accordingly, in one embodiment, the process of the present invention for producing fumarate in a non-human organism, like a microorganism or a plant or a part thereof, comprises increasing or generating the activity of a gene product with the activity of a gene product conferring the activity of “CDP-diacylglycerol-serine O-phosphatidyltransferase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said AvinDRAFT_{—}0035, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}0035, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}0035, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}0035, and preferably the activity is increased non-targeted, whereby the respective line disclose in column 7 the fine chemical fumarate.

Accordingly, in one embodiment, the molecule which activity is to be increased in the process of the invention is the gene product with an activity as a “CDP-diacylglycerol-serine O-phosphatidyltransferase”, preferably it is the molecule of section (a) or (b) of this paragraph.

In particular, it was observed that in plants, especially in Arabidopsis thaliana, increasing or generating the activity of a gene product non-targeted with the activity of a “CDP-diacylglycerolserine O-phosphatidyltransferase”, preferably being encoded by a gene comprising the nucleic acid sequence SEQ ID NO. 70583, preferably the coding region thereof, conferred the production of or the increase in fumarate compared with the wild type control.

The nucleic acid sequence of AvinDRAFT_{—}0035 from Azotobacter vinelandii, e.g. as shown in the respective line in column 5 of Table I, application no. 15, has been published in D. And the activity of the gene product thereof is the activity of CDP-diacylglycerol-serine O-phosphatidyltransferase.

Accordingly, in one embodiment, the process of the present invention for producing malate in a non-human organism, like a microorganism or a plant or a part thereof, comprises increasing or generating the activity of a gene product with the activity of a gene product conferring the activity of “CDP-diacylglycerol-serine O-phosphatidyltransferase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said AvinDRAFT_{—}0035, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}0035, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}0035, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}0035, and preferably the activity is increased non-targeted, whereby the respective line disclose in column 7 the fine chemical malate.

Accordingly, in one embodiment, the molecule which activity is to be increased in the process of the invention is the gene product with an activity as a “CDP-diacylglycerol-serine O-phosphatidyltransferase”, preferably it is the molecule of section (a) or (b) of this paragraph.

In particular, it was observed that in plants, especially in Arabidopsis thaliana, increasing or generating the activity of a gene product non-targeted with the activity of a “CDP-diacylglycerolserine O-phosphatidyltransferase”, preferably being encoded by a gene comprising the nucleic acid sequence SEQ ID NO. 70583, preferably the coding region thereof, conferred the production of or the increase in malate compared with the wild type control.

The nucleic acid sequence of AVINDRAFT_{—}0539 from Azotobacter vinelandii, e.g. as shown in the respective line in column 5 of Table I, application no. 15, has been published in D. And the activity of the gene product thereof is the activity of Glycine cleavage system T aminomethyltransferase.

Accordingly, in one embodiment, the process of the present invention for producing malate in a non-human organism, like a microorganism or a plant or a part thereof, comprises increasing or generating the activity of a gene product with the activity of a gene product conferring the activity of “Glycine cleavage system T aminomethyltransferase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said AVINDRAFT_{—}0539, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AVINDRAFT_{—}0539, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AVINDRAFT_{—}0539, or a functional equivalent or a homolog thereof as depicted in column 8 of Table II, application no. 15, preferably a homolog or functional equivalent as depicted in column 8 of Table II B, application no. 15, and being depicted in the same respective line as said AVINDRAFT_{—}0539, and preferably the activity is increased non-targeted, whereby the respective line disclose in column 7 the fine chemical malate.

Accordingly, in one embodiment, the molecule which activity is to be increased in the process of the invention is the gene product with an activity as a “Glycine cleavage system T aminomethyltransferase”, preferably it is the molecule of section (a) or (b) of this paragraph.

In particular, it was observed that in plants, especially in Arabidopsis thaliana, increasing or generating the activity of a gene product non-targeted with the activity of a “Glycine cleavage system T aminomethyltransferase”, preferably being encoded by a gene comprising the nucleic acid sequence SEQ ID NO. 70715, preferably the coding region thereof, conferred the production of or the increase in malate compared with the wild type control.

The nucleic acid sequence of AvinDRAFT_{—}1045 from Azotobacter vinelandii, e.g. as shown in the respective line in column 5 of Table I, application no. 15, has been published in D. And the activity of the gene product thereof is the activity of transcriptional regulator.

Accordingly, in one embodiment, the process of the present invention for producing malate in a non-human organism, like a microorganism or a plant or a part thereof, comprises increasing or generating the activity of a gene product with the activity of a gene product conferring the activity of “transcriptional regulator”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said AvinDRAFT_{—}1045, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}1045, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}1045, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}1045, and preferably the activity is increased non-targeted, whereby the respective line disclose in column 7 the fine chemical malate.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 25676, preferably the coding region thereof, conferred the production of or the increase in malate compared with the wild type control.

The nucleic acid sequence of AvinDRAFT_{—}1398 from Azotobacter vinelandii, e.g. as shown in the respective line in column 5 of Table I, application no. 15, has been published in D. And the activity of the gene product thereof is the activity of adenylylsulfate kinase.

Accordingly, in one embodiment, the process of the present invention for producing malate in a non-human organism, like a microorganism or a plant or a part thereof, comprises increasing or generating the activity of a gene product with the activity of a gene product conferring the activity of “adenylylsulfate kinase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said AvinDRAFT_{—}1398, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}1398, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}1398, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}1398, and preferably the activity is increased non-targeted, whereby the respective line disclose in column 7 the fine chemical malate.

Accordingly, in one embodiment, the molecule which activity is to be increased in the process of the invention is the gene product with an activity as a “adenylylsulfate kinase”, preferably it is the molecule of section (a) or (b) of this paragraph.

In particular, it was observed that in plants, especially in Arabidopsis thaliana, increasing or generating the activity of a gene product non-targeted with the activity of a “adenylylsulfate kinase”, preferably being encoded by a gene comprising the nucleic acid sequence SEQ ID NO. 25780, preferably the coding region thereof, conferred the production of or the increase in malate compared with the wild type control.

The nucleic acid sequence of AvinDRAFT_{—}1398 from Azotobacter vinelandii, e.g. as shown in the respective line in column 5 of Table I, application no. 15, has been published in D. And the activity of the gene product thereof is the activity of adenylylsulfate kinase.

Accordingly, in one embodiment, the process of the present invention for producing succinate in a non-human organism, like a microorganism or a plant or a part thereof, comprises increasing or generating the activity of a gene product with the activity of a gene product conferring the activity of “adenylylsulfate kinase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said AvinDRAFT_{—}1398, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}1398, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}1398, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}1398, and preferably the activity is increased non-targeted, whereby the respective line disclose in column 7 the fine chemical succinate.

Accordingly, in one embodiment, the molecule which activity is to be increased in the process of the invention is the gene product with an activity as a “adenylylsulfate kinase”, preferably it is the molecule of section (a) or (b) of this paragraph.

In particular, it was observed that in plants, especially in Arabidopsis thaliana, increasing or generating the activity of a gene product non-targeted with the activity of a “adenylylsulfate kinase”, preferably being encoded by a gene comprising the nucleic acid sequence SEQ ID NO. 25780, preferably the coding region thereof, conferred the production of or the increase in succinate compared with the wild type control.

The nucleic acid sequence of AvinDRAFT_{—}1624 from Azotobacter vinelandii, e.g. as shown in the respective line in column 5 of Table I, application no. 15, has been published in D. And the activity of the gene product thereof is the activity of sec-independent protein translocase.

Accordingly, in one embodiment, the process of the present invention for producing fumarate in a non-human organism, like a microorganism or a plant or a part thereof, comprises increasing or generating the activity of a gene product with the activity of a gene product conferring the activity of “sec-independent protein translocase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said AvinDRAFT_{—}1624, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}1624, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}1624, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}1624, and preferably the activity is increased non-targeted, whereby the respective line disclose in column 7 the fine chemical fumarate.

Accordingly, in one embodiment, the molecule which activity is to be increased in the process of the invention is the gene product with an activity as a “sec-independent protein translocase”, preferably it is the molecule of section (a) or (b) of this paragraph.

In particular, it was observed that in plants, especially in Arabidopsis thaliana, increasing or generating the activity of a gene product non-targeted with the activity of a “sec-independent protein translocase”, preferably being encoded by a gene comprising the nucleic acid sequence SEQ ID NO. 26196, preferably the coding region thereof, conferred the production of or the increase in fumarate compared with the wild type control.

The nucleic acid sequence of AvinDRAFT_{—}1624 from Azotobacter vinelandii, e.g. as shown in the respective line in column 5 of Table I, application no. 15, has been published in D. And the activity of the gene product thereof is the activity of sec-independent protein translocase.

Accordingly, in one embodiment, the process of the present invention for producing malate in a non-human organism, like a microorganism or a plant or a part thereof, comprises increasing or generating the activity of a gene product with the activity of a gene product conferring the activity of “sec-independent protein translocase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said AvinDRAFT_{—}1624, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}1624, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}1624, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}1624, and preferably the activity is increased non-targeted, whereby the respective line disclose in column 7 the fine chemical malate.

Accordingly, in one embodiment, the molecule which activity is to be increased in the process of the invention is the gene product with an activity as a “sec-independent protein translocase”, preferably it is the molecule of section (a) or (b) of this paragraph.

In particular, it was observed that in plants, especially in Arabidopsis thaliana, increasing or generating the activity of a gene product non-targeted with the activity of a “sec-independent protein translocase”, preferably being encoded by a gene comprising the nucleic acid sequence SEQ ID NO. 26196, preferably the coding region thereof, conferred the production of or the increase in malate compared with the wild type control.

The nucleic acid sequence of AvinDRAFT_{—}1699 from Azotobacter vinelandii, e.g. as shown in the respective line in column 5 of Table I, application no. 15, has been published in D. And the activity of the gene product thereof is the activity of fumarate hydratase.

Accordingly, in one embodiment, the process of the present invention for producing fumarate in a non-human organism, like a microorganism or a plant or a part thereof, comprises increasing or generating the activity of a gene product with the activity of a gene product conferring the activity of “fumarate hydratase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said AvinDRAFT_{—}1699, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}1699, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}1699, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}1699, and preferably the activity is increased non-targeted, whereby the respective line disclose in column 7 the fine chemical fumarate.

Accordingly, in one embodiment, the molecule which activity is to be increased in the process of the invention is the gene product with an activity as a “fumarate hydratase”, preferably it is the molecule of section (a) or (b) of this paragraph.

In particular, it was observed that in plants, especially in Arabidopsis thaliana, increasing or generating the activity of a gene product non-targeted with the activity of a “fumarate hydratase”, preferably being encoded by a gene comprising the nucleic acid sequence SEQ ID NO. 133719, preferably the coding region thereof, conferred the production of or the increase in fumarate compared with the wild type control.

The nucleic acid sequence of AvinDRAFT_{—}1806 from Azotobacter vinelandii, e.g. as shown in the respective line in column 5 of Table I, application no. 15, has been published in D. And the activity of the gene product thereof is the activity of pyruvate kinase.

Accordingly, in one embodiment, the process of the present invention for producing succinate in a non-human organism, like a microorganism or a plant or a part thereof, comprises increasing or generating the activity of a gene product with the activity of a gene product conferring the activity of “pyruvate kinase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said AvinDRAFT_{—}1806, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}1806, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}1806, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}1806, and preferably the activity is increased non-targeted, whereby the respective line disclose in column 7 the fine chemical succinate.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product non-targeted with the activity of a “pyruvate kinase”, preferably being encoded by a gene comprising the nucleic acid sequence SEQ ID NO. 26434, preferably the coding region thereof, conferred the production of or the increase in succinate compared with the wild type control.

The nucleic acid sequence of AvinDRAFT_{—}2010 from Azotobacter vinelandii, e.g. as shown in the respective line in column 5 of Table I, application no. 15, has been published in D. And the activity of the gene product thereof is the activity of transcriptional regulator protein.

Accordingly, in one embodiment, the process of the present invention for producing succinate in a non-human organism, like a microorganism or a plant or a part thereof, comprises increasing or generating the activity of a gene product with the activity of a gene product conferring the activity of “transcriptional regulator protein”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said AvinDRAFT_{—}2010, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}2010, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}2010, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}2010, and preferably the activity is increased non-targeted, whereby the respective line disclose in column 7 the fine chemical succinate.

Accordingly, in one embodiment, the molecule which activity is to be increased in the process of the invention is the gene product with an activity as a “transcriptional regulator protein”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator protein”, preferably being encoded by a gene comprising the nucleic acid sequence SEQ ID NO. 114198, preferably the coding region thereof, conferred the production of or the increase in succinate compared with the wild type control.

The nucleic acid sequence of AvinDRAFT_{—}2091 from Azotobacter vinelandii, e.g. as shown in the respective line in column 5 of Table I, application no. 15, has been published in D. And the activity of the gene product thereof is the activity of beta-hydroxylase.

Accordingly, in one embodiment, the process of the present invention for producing glycerate in a non-human organism, like a microorganism or a plant or a part thereof, comprises increasing or generating the activity of a gene product with the activity of a gene product conferring the activity of “beta-hydroxylase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical glycerate), application no. 15, preferably the coding region thereof, or a homolog or a fragment thereof, and being depicted in the same respective line as said AvinDRAFT_{—}2091, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}2091, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}2091, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}2091, and preferably the activity is increased non-targeted, whereby the respective line disclose in column 7 the fine chemical glycerate.

Accordingly, in one embodiment, the molecule which activity is to be increased in the process of the invention is the gene product with an activity as a “beta-hydroxylase”, preferably it is the molecule of section (a) or (b) of this paragraph.

In particular, it was observed that in plants, especially in Arabidopsis thaliana, increasing or generating the activity of a gene product non-targeted with the activity of a “beta-hydroxylase”, preferably being encoded by a gene comprising the nucleic acid sequence SEQ ID NO. 6040, preferably the coding region thereof, conferred the production of or the increase in glycerate compared with the wild type control.

The nucleic acid sequence of AvinDRAFT_{—}2365 from Azotobacter vinelandii, e.g. as shown in the respective line in column 5 of Table I, application no. 15, has been published in D. And the activity of the gene product thereof is the activity of 30S ribosomal protein S3.

Accordingly, in one embodiment, the process of the present invention for producing succinate in a non-human organism, like a microorganism or a plant or a part thereof, comprises increasing or generating the activity of a gene product with the activity of a gene product conferring the activity of “30S ribosomal protein S3”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said AvinDRAFT_{—}2365, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}2365, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}2365, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}2365, and preferably the activity is increased non-targeted, whereby the respective line disclose in column 7 the fine chemical succinate.

Accordingly, in one embodiment, the molecule which activity is to be increased in the process of the invention is the gene product with an activity as a “30S ribosomal protein S3”, preferably it is the molecule of section (a) or (b) of this paragraph.

In particular, it was observed that in plants, especially in Arabidopsis thaliana, increasing or generating the activity of a gene product non-targeted with the activity of a “30S ribosomal protein S3”, preferably being encoded by a gene comprising the nucleic acid sequence SEQ ID NO. 71325, preferably the coding region thereof, conferred the production of or the increase in succinate compared with the wild type control.

The nucleic acid sequence of AvinDRAFT_{—}2369 from Azotobacter vinelandii, e.g. as shown in the respective line in column 5 of Table I, application no. 15, has been published in D. And the activity of the gene product thereof is the activity of 50S ribosomal protein L14.

Accordingly, in one embodiment, the process of the present invention for producing citramalate in a non-human organism, like a microorganism or a plant or a part thereof, comprises increasing or generating the activity of a gene product with the activity of a gene product conferring the activity of “50S ribosomal protein L14”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said AvinDRAFT_{—}2369, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}2369, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}2369, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}2369, and preferably the activity is increased non-targeted, whereby the respective line disclose in column 7 the fine chemical citramalate.

Accordingly, in one embodiment, the molecule which activity is to be increased in the process of the invention is the gene product with an activity as a “505 ribosomal protein L14”, preferably it is the molecule of section (a) or (b) of this paragraph.

In particular, it was observed that in plants, especially in Arabidopsis thaliana, increasing or generating the activity of a gene product non-targeted with the activity of a “505 ribosomal protein L14”, preferably being encoded by a gene comprising the nucleic acid sequence SEQ ID NO. 71763, preferably the coding region thereof, conferred the production of or the increase in citramalate compared with the wild type control.

The nucleic acid sequence of AvinDRAFT_{—}2754 from Azotobacter vinelandii, e.g. as shown in the respective line in column 5 of Table I, application no. 15, has been published in D. And the activity of the gene product thereof is the activity of acyl-CoA synthase.

Accordingly, in one embodiment, the process of the present invention for producing malate in a non-human organism, like a microorganism or a plant or a part thereof, comprises increasing or generating the activity of a gene product with the activity of a gene product conferring the activity of “acyl-CoA synthase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said AvinDRAFT_{—}2754, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}2754, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}2754, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}2754, and preferably the activity is increased non-targeted, whereby the respective line disclose in column 7 the fine chemical malate.

Accordingly, in one embodiment, the molecule which activity is to be increased in the process of the invention is the gene product with an activity as a “acyl-CoA synthase”, preferably it is the molecule of section (a) or (b) of this paragraph.

In particular, it was observed that in plants, especially in Arabidopsis thaliana, increasing or generating the activity of a gene product non-targeted with the activity of a “acyl-CoA synthase”, preferably being encoded by a gene comprising the nucleic acid sequence SEQ ID NO. 28040, preferably the coding region thereof, conferred the production of or the increase in malate compared with the wild type control.

The nucleic acid sequence of AvinDRAFT_{—}3028 from Azotobacter vinelandii, e.g. as shown in the respective line in column 5 of Table I, application no. 15, has been published in D. And the activity of the gene product thereof is the activity of 2-oxoglutarate dehydrogenase E1 subunit.

Accordingly, in one embodiment, the process of the present invention for producing citramalate in a non-human organism, like a microorganism or a plant or a part thereof, comprises increasing or generating the activity of a gene product with the activity of a gene product conferring the activity of “2-oxoglutarate dehydrogenase E1 subunit”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said AvinDRAFT_{—}3028, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}3028, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}3028, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}3028, and preferably the activity is increased non-targeted, whereby the respective line disclose in column 7 the fine chemical citramalate.

Accordingly, in one embodiment, the molecule which activity is to be increased in the process of the invention is the gene product with an activity as a “2-oxoglutarate dehydrogenase E1 subunit”, preferably it is the molecule of section (a) or (b) of this paragraph.

In particular, it was observed that in plants, especially in Arabidopsis thaliana, increasing or generating the activity of a gene product non-targeted with the activity of a “2-oxoglutarate dehydrogenase E1 subunit”, preferably being encoded by a gene comprising the nucleic acid sequence SEQ ID NO. 6075, preferably the coding region thereof, conferred the production of or the increase in citramalate compared with the wild type control.

The nucleic acid sequence of AvinDRAFT_{—}3028 from Azotobacter vinelandii, e.g. as shown in the respective line in column 5 of Table I, application no. 15, has been published in D. And the activity of the gene product thereof is the activity of 2-oxoglutarate dehydrogenase E1 subunit.

Accordingly, in one embodiment, the process of the present invention for producing fumarate in a non-human organism, like a microorganism or a plant or a part thereof, comprises increasing or generating the activity of a gene product with the activity of a gene product conferring the activity of “2-oxoglutarate dehydrogenase E1 subunit”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said AvinDRAFT_{—}3028, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}3028, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}3028, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}3028, and preferably the activity is increased non-targeted, whereby the respective line disclose in column 7 the fine chemical fumarate.

Accordingly, in one embodiment, the molecule which activity is to be increased in the process of the invention is the gene product with an activity as a “2-oxoglutarate dehydrogenase E1 subunit”, preferably it is the molecule of section (a) or (b) of this paragraph.

In particular, it was observed that in plants, especially in Arabidopsis thaliana, increasing or generating the activity of a gene product non-targeted with the activity of a “2-oxoglutarate dehydrogenase E1 subunit”, preferably being encoded by a gene comprising the nucleic acid sequence SEQ ID NO. 6075, preferably the coding region thereof, conferred the production of or the increase in fumarate compared with the wild type control.

The nucleic acid sequence of AvinDRAFT_{—}3028 from Azotobacter vinelandii, e.g. as shown in the respective line in column 5 of Table I, application no. 15, has been published in D. And the activity of the gene product thereof is the activity of 2-oxoglutarate dehydrogenase E1 subunit.

Accordingly, in one embodiment, the process of the present invention for producing malate in a non-human organism, like a microorganism or a plant or a part thereof, comprises increasing or generating the activity of a gene product with the activity of a gene product conferring the activity of “2-oxoglutarate dehydrogenase E1 subunit”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said AvinDRAFT_{—}3028, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}3028, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}3028, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}3028, and preferably the activity is increased non-targeted, whereby the respective line disclose in column 7 the fine chemical malate.

Accordingly, in one embodiment, the molecule which activity is to be increased in the process of the invention is the gene product with an activity as a “2-oxoglutarate dehydrogenase E1 subunit”, preferably it is the molecule of section (a) or (b) of this paragraph.

In particular, it was observed that in plants, especially in Arabidopsis thaliana, increasing or generating the activity of a gene product non-targeted with the activity of a “2-oxoglutarate dehydrogenase E1 subunit”, preferably being encoded by a gene comprising the nucleic acid sequence SEQ ID NO. 6075, preferably the coding region thereof, conferred the production of or the increase in malate compared with the wild type control.

The nucleic acid sequence of AvinDRAFT_{—}3028 from Azotobacter vinelandii, e.g. as shown in the respective line in column 5 of Table I, application no. 15, has been published in D. And the activity of the gene product thereof is the activity of 2-oxoglutarate dehydrogenase E1 subunit.

Accordingly, in one embodiment, the process of the present invention for producing succinate in a non-human organism, like a microorganism or a plant or a part thereof, comprises increasing or generating the activity of a gene product with the activity of a gene product conferring the activity of “2-oxoglutarate dehydrogenase E1 subunit”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said AvinDRAFT_{—}3028, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}3028, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}3028, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}3028, and preferably the activity is increased non-targeted, whereby the respective line disclose in column 7 the fine chemical succinate.

Accordingly, in one embodiment, the molecule which activity is to be increased in the process of the invention is the gene product with an activity as a “2-oxoglutarate dehydrogenase E1 subunit”, preferably it is the molecule of section (a) or (b) of this paragraph.

In particular, it was observed that in plants, especially in Arabidopsis thaliana, increasing or generating the activity of a gene product non-targeted with the activity of a “2-oxoglutarate dehydrogenase E1 subunit”, preferably being encoded by a gene comprising the nucleic acid sequence SEQ ID NO. 6075, preferably the coding region thereof, conferred the production of or the increase in succinate compared with the wild type control.

The nucleic acid sequence of AvinDRAFT_{—}3028 from Azotobacter vinelandii, e.g. as shown in the respective line in column 5 of Table I, application no. 15, has been published in D. And the activity of the gene product thereof is the activity of 2-oxoglutarate dehydrogenase E1 subunit.

Accordingly, in one embodiment, the process of the present invention for producing vitamin c and/or threonic acid in a non-human organism, like a microorganism or a plant or a part thereof, comprises increasing or generating the activity of a gene product with the activity of a gene product conferring the activity of “2-oxoglutarate dehydrogenase E1 subunit”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical vitamin c and/or threonic acid), application no. 15, preferably the coding region thereof, or a homolog or a fragment thereof, and being depicted in the same respective line as said AvinDRAFT_{—}3028, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}3028, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}3028, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}3028, and preferably the activity is increased non-targeted, whereby the respective line disclose in column 7 the fine chemical vitamin c and/or threonic acid.

Accordingly, in one embodiment, the molecule which activity is to be increased in the process of the invention is the gene product with an activity as a “2-oxoglutarate dehydrogenase E1 subunit”, preferably it is the molecule of section (a) or (b) of this paragraph.

In particular, it was observed that in plants, especially in Arabidopsis thaliana, increasing or generating the activity of a gene product non-targeted with the activity of a “2-oxoglutarate dehydrogenase E1 subunit”, preferably being encoded by a gene comprising the nucleic acid sequence SEQ ID NO. 6075, preferably the coding region thereof, conferred the production of or the increase in vitamin c and/or threonic acid compared with the wild type control.

The nucleic acid sequence of AvinDRAFT_{—}3186 from Azotobacter vinelandii, e.g. as shown in the respective line in column 5 of Table I, application no. 15, has been published in D. And the activity of the gene product thereof is the activity of transcriptional regulator.

Accordingly, in one embodiment, the process of the present invention for producing citramalate in a non-human organism, like a microorganism or a plant or a part thereof, comprises increasing or generating the activity of a gene product with the activity of a gene product conferring the activity of “transcriptional regulator”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said AvinDRAFT_{—}3186, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}3186, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}3186, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}3186, and preferably the activity is increased non-targeted, whereby the respective line disclose in column 7 the fine chemical citramalate.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 29246, preferably the coding region thereof, conferred the production of or the increase in citramalate compared with the wild type control.

The nucleic acid sequence of AvinDRAFT_{—}3186 from Azotobacter vinelandii, e.g. as shown in the respective line in column 5 of Table I, application no. 15, has been published in D. And the activity of the gene product thereof is the activity of transcriptional regulator.

Accordingly, in one embodiment, the process of the present invention for producing fumarate in a non-human organism, like a microorganism or a plant or a part thereof, comprises increasing or generating the activity of a gene product with the activity of a gene product conferring the activity of “transcriptional regulator”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said AvinDRAFT_{—}3186, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}3186, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}3186, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}3186, and preferably the activity is increased non-targeted, whereby the respective line disclose in column 7 the fine chemical fumarate.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 29246, preferably the coding region thereof, conferred the production of or the increase in fumarate compared with the wild type control.

The nucleic acid sequence of AvinDRAFT_{—}3253 from Azotobacter vinelandii, e.g. as shown in the respective line in column 5 of Table I, application no. 15, has been published in D. And the activity of the gene product thereof is the activity of oxidoreductase.

Accordingly, in one embodiment, the process of the present invention for producing succinate in a non-human organism, like a microorganism or a plant or a part thereof, comprises increasing or generating the activity of a gene product with the activity of a gene product conferring the activity of “oxidoreductase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said AvinDRAFT_{—}3253, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}3253, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}3253, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}3253, and preferably the activity is increased non-targeted, whereby the respective line disclose in column 7 the fine chemical succinate.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 29500, preferably the coding region thereof, conferred the production of or the increase in succinate compared with the wild type control.

The nucleic acid sequence of AvinDRAFT_{—}3605 from Azotobacter vinelandii, e.g. as shown in the respective line in column 5 of Table I, application no. 15, has been published in D. And the activity of the gene product thereof is the activity of CTP synthase.

Accordingly, in one embodiment, the process of the present invention for producing fumarate in a non-human organism, like a microorganism or a plant or a part thereof, comprises increasing or generating the activity of a gene product with the activity of a gene product conferring the activity of “CTP synthase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said AvinDRAFT_{—}3605, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}3605, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}3605, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}3605, and preferably the activity is increased non-targeted, whereby the respective line disclose in column 7 the fine chemical fumarate.

Accordingly, in one embodiment, the molecule which activity is to be increased in the process of the invention is the gene product with an activity as a “CTP synthase”, preferably it is the molecule of section (a) or (b) of this paragraph.

In particular, it was observed that in plants, especially in Arabidopsis thaliana, increasing or generating the activity of a gene product non-targeted with the activity of a “CTP synthase”, preferably being encoded by a gene comprising the nucleic acid sequence SEQ ID NO. 31026, preferably the coding region thereof, conferred the production of or the increase in fumarate compared with the wild type control.

The nucleic acid sequence of AvinDRAFT_{—}3605 from Azotobacter vinelandii, e.g. as shown in the respective line in column 5 of Table I, application no. 15, has been published in D. And the activity of the gene product thereof is the activity of CTP synthase.

Accordingly, in one embodiment, the process of the present invention for producing malate in a non-human organism, like a microorganism or a plant or a part thereof, comprises increasing or generating the activity of a gene product with the activity of a gene product conferring the activity of “CTP synthase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said AvinDRAFT_{—}3605, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}3605, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}3605, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}3605, and preferably the activity is increased non-targeted, whereby the respective line disclose in column 7 the fine chemical malate.

Accordingly, in one embodiment, the molecule which activity is to be increased in the process of the invention is the gene product with an activity as a “CTP synthase”, preferably it is the molecule of section (a) or (b) of this paragraph.

In particular, it was observed that in plants, especially in Arabidopsis thaliana, increasing or generating the activity of a gene product non-targeted with the activity of a “CTP synthase”, preferably being encoded by a gene comprising the nucleic acid sequence SEQ ID NO. 31026, preferably the coding region thereof, conferred the production of or the increase in malate compared with the wild type control.

The nucleic acid sequence of AvinDRAFT_{—}3605 from Azotobacter vinelandii, e.g. as shown in the respective line in column 5 of Table I, application no. 15, has been published in D. And the activity of the gene product thereof is the activity of CTP synthase.

Accordingly, in one embodiment, the process of the present invention for producing succinate in a non-human organism, like a microorganism or a plant or a part thereof, comprises increasing or generating the activity of a gene product with the activity of a gene product conferring the activity of “CTP synthase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said AvinDRAFT_{—}3605, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}3605, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}3605, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}3605, and preferably the activity is increased non-targeted, whereby the respective line disclose in column 7 the fine chemical succinate.

Accordingly, in one embodiment, the molecule which activity is to be increased in the process of the invention is the gene product with an activity as a “CTP synthase”, preferably it is the molecule of section (a) or (b) of this paragraph.

In particular, it was observed that in plants, especially in Arabidopsis thaliana, increasing or generating the activity of a gene product non-targeted with the activity of a “CTP synthase”, preferably being encoded by a gene comprising the nucleic acid sequence SEQ ID NO. 31026, preferably the coding region thereof, conferred the production of or the increase in succinate compared with the wild type control.

The nucleic acid sequence of AvinDRAFT_{—}3629 from Azotobacter vinelandii, e.g. as shown in the respective line in column 5 of Table I, application no. 15, has been published in D. And the activity of the gene product thereof is the activity of recombinase A.

Accordingly, in one embodiment, the process of the present invention for producing succinate in a non-human organism, like a microorganism or a plant or a part thereof, comprises increasing or generating the activity of a gene product with the activity of a gene product conferring the activity of “recombinase A”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said AvinDRAFT_{—}3629, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}3629, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}3629, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}3629, and preferably the activity is increased non-targeted, whereby the respective line disclose in column 7 the fine chemical succinate.

Accordingly, in one embodiment, the molecule which activity is to be increased in the process of the invention is the gene product with an activity as a “recombinase A”, preferably it is the molecule of section (a) or (b) of this paragraph.

In particular, it was observed that in plants, especially in Arabidopsis thaliana, increasing or generating the activity of a gene product non-targeted with the activity of a “recombinase A”, preferably being encoded by a gene comprising the nucleic acid sequence SEQ ID NO. 114231, preferably the coding region thereof, conferred the production of or the increase in succinate compared with the wild type control.

The nucleic acid sequence of AvinDRAFT_{—}4384 from Azotobacter vinelandii, e.g. as shown in the respective line in column 5 of Table I, application no. 15, has been published in D. And the activity of the gene product thereof is the activity of glucose-1-phosphate cytidylyltransferase.

Accordingly, in one embodiment, the process of the present invention for producing malate in a non-human organism, like a microorganism or a plant or a part thereof, comprises increasing or generating the activity of a gene product with the activity of a gene product conferring the activity of “glucose-1-phosphate cytidylyltransferase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said AvinDRAFT_{—}4384, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}4384, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}4384, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}4384, and preferably the activity is increased non-targeted, whereby the respective line disclose in column 7 the fine chemical malate.

Accordingly, in one embodiment, the molecule which activity is to be increased in the process of the invention is the gene product with an activity as a “glucose-1-phosphate cytidylyltransferase”, preferably it is the molecule of section (a) or (b) of this paragraph.

In particular, it was observed that in plants, especially in Arabidopsis thaliana, increasing or generating the activity of a gene product non-targeted with the activity of a “glucose-1-phosphate cytidylyltransferase”, preferably being encoded by a gene comprising the nucleic acid sequence SEQ ID NO. 31717, preferably the coding region thereof, conferred the production of or the increase in malate compared with the wild type control.

The nucleic acid sequence of AvinDRAFT_{—}4385 from Azotobacter vinelandii, e.g. as shown in the respective line in column 5 of Table I, application no. 15, has been published in D. And the activity of the gene product thereof is the activity of CDP-glucose-4,6-dehydratase.

Accordingly, in one embodiment, the process of the present invention for producing fumarate in a non-human organism, like a microorganism or a plant or a part thereof, comprises increasing or generating the activity of a gene product with the activity of a gene product conferring the activity of “CDP-glucose-4,6-dehydratase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said AvinDRAFT_{—}4385, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}4385, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}4385, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}4385, and preferably the activity is increased non-targeted, whereby the respective line disclose in column 7 the fine chemical fumarate.

Accordingly, in one embodiment, the molecule which activity is to be increased in the process of the invention is the gene product with an activity as a “CDP-glucose-4,6-dehydratase”, preferably it is the molecule of section (a) or (b) of this paragraph.

In particular, it was observed that in plants, especially in Arabidopsis thaliana, increasing or generating the activity of a gene product non-targeted with the activity of a “CDP-glucose-4,6-dehydratase”, preferably being encoded by a gene comprising the nucleic acid sequence SEQ ID NO. 72935, preferably the coding region thereof, conferred the production of or the increase in fumarate compared with the wild type control.

The nucleic acid sequence of AvinDRAFT_{—}4385 from Azotobacter vinelandii, e.g. as shown in the respective line in column 5 of Table I, application no. 15, has been published in D. And the activity of the gene product thereof is the activity of CDP-glucose-4,6-dehydratase.

Accordingly, in one embodiment, the process of the present invention for producing malate in a non-human organism, like a microorganism or a plant or a part thereof, comprises increasing or generating the activity of a gene product with the activity of a gene product conferring the activity of “CDP-glucose-4,6-dehydratase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said AvinDRAFT_{—}4385, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}4385, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}4385, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}4385, and preferably the activity is increased non-targeted, whereby the respective line disclose in column 7 the fine chemical malate.

Accordingly, in one embodiment, the molecule which activity is to be increased in the process of the invention is the gene product with an activity as a “CDP-glucose-4,6-dehydratase”, preferably it is the molecule of section (a) or (b) of this paragraph.

In particular, it was observed that in plants, especially in Arabidopsis thaliana, increasing or generating the activity of a gene product non-targeted with the activity of a “CDP-glucose-4,6-dehydratase”, preferably being encoded by a gene comprising the nucleic acid sequence SEQ ID NO. 72935, preferably the coding region thereof, conferred the production of or the increase in malate compared with the wild type control.

The nucleic acid sequence of AvinDRAFT_{—}4562 from Azotobacter vinelandii, e.g. as shown in the respective line in column 5 of Table I, application no. 15, has been published in D. And the activity of the gene product thereof is the activity of aminotransferase.

Accordingly, in one embodiment, the process of the present invention for producing malate in a non-human organism, like a microorganism or a plant or a part thereof, comprises increasing or generating the activity of a gene product with the activity of a gene product conferring the activity of “aminotransferase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said AvinDRAFT_{—}4562, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}4562, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}4562, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}4562, and preferably the activity is increased non-targeted, whereby the respective line disclose in column 7 the fine chemical malate.

Accordingly, in one embodiment, the molecule which activity is to be increased in the process of the invention is the gene product with an activity as a “aminotransferase”, preferably it is the molecule of section (a) or (b) of this paragraph.

In particular, it was observed that in plants, especially in Arabidopsis thaliana, increasing or generating the activity of a gene product non-targeted with the activity of a “aminotransferase”, preferably being encoded by a gene comprising the nucleic acid sequence SEQ ID NO. 31926, preferably the coding region thereof, conferred the production of or the increase in malate compared with the wild type control.

The nucleic acid sequence of AvinDRAFT_{—}4594 from Azotobacter vinelandii, e.g. as shown in the respective line in column 5 of Table I, application no. 15, has been published in D. And the activity of the gene product thereof is the activity of isocitrate lyase.

Accordingly, in one embodiment, the process of the present invention for producing succinate in a non-human organism, like a microorganism or a plant or a part thereof, comprises increasing or generating the activity of a gene product with the activity of a gene product conferring the activity of “isocitrate lyase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said AvinDRAFT_{—}4594, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}4594, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}4594, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}4594, and preferably the activity is increased non-targeted, whereby the respective line disclose in column 7 the fine chemical succinate.

Accordingly, in one embodiment, the molecule which activity is to be increased in the process of the invention is the gene product with an activity as a “isocitrate lyase”, preferably it is the molecule of section (a) or (b) of this paragraph.

In particular, it was observed that in plants, especially in Arabidopsis thaliana, increasing or generating the activity of a gene product non-targeted with the activity of a “isocitrate lyase”, preferably being encoded by a gene comprising the nucleic acid sequence SEQ ID NO. 134171, preferably the coding region thereof, conferred the production of or the increase in succinate compared with the wild type control.

The nucleic acid sequence of AvinDRAFT_{—}4606 from Azotobacter vinelandii, e.g. as shown in the respective line in column 5 of Table I, application no. 15, has been published in D. And the activity of the gene product thereof is the activity of NADH-quinone oxidoreductase subunit.

Accordingly, in one embodiment, the process of the present invention for producing fumarate in a non-human organism, like a microorganism or a plant or a part thereof, comprises increasing or generating the activity of a gene product with the activity of a gene product conferring the activity of “NADH-quinone oxidoreductase subunit”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said AvinDRAFT_{—}4606, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}4606, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}4606, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}4606, and preferably the activity is increased non-targeted, whereby the respective line disclose in column 7 the fine chemical fumarate.

Accordingly, in one embodiment, the molecule which activity is to be increased in the process of the invention is the gene product with an activity as a “NADH-quinone oxidoreductase subunit”, preferably it is the molecule of section (a) or (b) of this paragraph.

In particular, it was observed that in plants, especially in Arabidopsis thaliana, increasing or generating the activity of a gene product non-targeted with the activity of a “NADH-quinone oxidoreductase subunit”, preferably being encoded by a gene comprising the nucleic acid sequence SEQ ID NO. 73719, preferably the coding region thereof, conferred the production of or the increase in fumarate compared with the wild type control.

The nucleic acid sequence of AvinDRAFT_{—}4606 from Azotobacter vinelandii, e.g. as shown in the respective line in column 5 of Table I, application no. 15, has been published in D. And the activity of the gene product thereof is the activity of NADH-quinone oxidoreductase subunit.

Accordingly, in one embodiment, the process of the present invention for producing malate in a non-human organism, like a microorganism or a plant or a part thereof, comprises increasing or generating the activity of a gene product with the activity of a gene product conferring the activity of “NADH-quinone oxidoreductase subunit”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said AvinDRAFT_{—}4606, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}4606, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}4606, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}4606, and preferably the activity is increased non-targeted, whereby the respective line disclose in column 7 the fine chemical malate.

Accordingly, in one embodiment, the molecule which activity is to be increased in the process of the invention is the gene product with an activity as a “NADH-quinone oxidoreductase subunit”, preferably it is the molecule of section (a) or (b) of this paragraph.

In particular, it was observed that in plants, especially in Arabidopsis thaliana, increasing or generating the activity of a gene product non-targeted with the activity of a “NADH-quinone oxidoreductase subunit”, preferably being encoded by a gene comprising the nucleic acid sequence SEQ ID NO. 73719, preferably the coding region thereof, conferred the production of or the increase in malate compared with the wild type control.

The nucleic acid sequence of AvinDRAFT_{—}4989 from Azotobacter vinelandii, e.g. as shown in the respective line in column 5 of Table I, application no. 15, has been published in D. And the activity of the gene product thereof is the activity of RNA polymerase sigma factor.

Accordingly, in one embodiment, the process of the present invention for producing malate in a non-human organism, like a microorganism or a plant or a part thereof, comprises increasing or generating the activity of a gene product with the activity of a gene product conferring the activity of “RNA polymerase sigma factor”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said AvinDRAFT_{—}4989, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}4989, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}4989, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}4989, and preferably the activity is increased non-targeted, whereby the respective line disclose in column 7 the fine chemical malate.

Accordingly, in one embodiment, the molecule which activity is to be increased in the process of the invention is the gene product with an activity as a “RNA polymerase sigma factor”, preferably it is the molecule of section (a) or (b) of this paragraph.

In particular, it was observed that in plants, especially in Arabidopsis thaliana, increasing or generating the activity of a gene product non-targeted with the activity of a “RNA polymerase sigma factor”, preferably being encoded by a gene comprising the nucleic acid sequence SEQ ID NO. 74452, preferably the coding region thereof, conferred the production of or the increase in malate compared with the wild type control.

The nucleic acid sequence of AvinDRAFT_{—}5103 from Azotobacter vinelandii, e.g. as shown in the respective line in column 5 of Table I, application no. 15, has been published in D. And the activity of the gene product thereof is the activity of hydrolase.

Accordingly, in one embodiment, the process of the present invention for producing malate in a non-human organism, like a microorganism or a plant or a part thereof, comprises increasing or generating the activity of a gene product with the activity of a gene product conferring the activity of “hydrolase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said AvinDRAFT_{—}5103, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}5103, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}5103, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}5103, and preferably the activity is increased non-targeted, whereby the respective line disclose in column 7 the fine chemical malate.

Accordingly, in one embodiment, the molecule which activity is to be increased in the process of the invention is the gene product with an activity as a “hydrolase”, preferably it is the molecule of section (a) or (b) of this paragraph.

In particular, it was observed that in plants, especially in Arabidopsis thaliana, increasing or generating the activity of a gene product non-targeted with the activity of a “hydrolase”, preferably being encoded by a gene comprising the nucleic acid sequence SEQ ID NO. 6510, preferably the coding region thereof, conferred the production of or the increase in malate compared with the wild type control.

The nucleic acid sequence of AvinDRAFT_{—}5246 from Azotobacter vinelandii, e.g. as shown in the respective line in column 5 of Table I, application no. 15, has been published in D. And the activity of the gene product thereof is the activity of glutamate-ammonia-ligase.

Accordingly, in one embodiment, the process of the present invention for producing malate in a non-human organism, like a microorganism or a plant or a part thereof, comprises increasing or generating the activity of a gene product with the activity of a gene product conferring the activity of “glutamate-ammonia-ligase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said AvinDRAFT_{—}5246, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}5246, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}5246, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}5246, and preferably the activity is increased non-targeted, whereby the respective line disclose in column 7 the fine chemical malate.

Accordingly, in one embodiment, the molecule which activity is to be increased in the process of the invention is the gene product with an activity as a “glutamate-ammonia-ligase”, preferably it is the molecule of section (a) or (b) of this paragraph.

In particular, it was observed that in plants, especially in Arabidopsis thaliana, increasing or generating the activity of a gene product non-targeted with the activity of a “glutamate-ammonia-ligase”, preferably being encoded by a gene comprising the nucleic acid sequence SEQ ID NO. 32037, preferably the coding region thereof, conferred the production of or the increase in malate compared with the wild type control.

The nucleic acid sequence of AvinDRAFT_{—}6679 from Azotobacter vinelandii, e.g. as shown in the respective line in column 5 of Table I, application no. 15, has been published in D. And the activity of the gene product thereof is the activity of electron transfer flavoprotein subunit beta.

Accordingly, in one embodiment, the process of the present invention for producing succinate in a non-human organism, like a microorganism or a plant or a part thereof, comprises increasing or generating the activity of a gene product with the activity of a gene product conferring the activity of “electron transfer flavoprotein subunit beta”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said AvinDRAFT_{—}6679, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AvinDRAFT_{—}6679, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AvinDRAFT_{—}6679, or a functional equivalent or a homolog thereof as depicted in column 8 of Table II, application no. 15, preferably a homolog or functional equivalent as depicted in column 8 of Table II B, application no. 15, and being depicted in the same respective line as said AvinDRAFT_{—}6679, and preferably the activity is increased non-targeted, whereby the respective line disclose in column 7 the fine chemical succinate.

Accordingly, in one embodiment, the molecule which activity is to be increased in the process of the invention is the gene product with an activity as a “electron transfer flavoprotein subunit beta”, preferably it is the molecule of section (a) or (b) of this paragraph.

In particular, it was observed that in plants, especially in Arabidopsis thaliana, increasing or generating the activity of a gene product non-targeted with the activity of a “electron transfer flavoprotein subunit beta”, preferably being encoded by a gene comprising the nucleic acid sequence SEQ ID NO. 74729, preferably the coding region thereof, conferred the production of or the increase in succinate compared with the wild type control.

The nucleic acid sequence of AX653549 from Oryza sativa, e.g. as shown in the respective line in column 5 of Table I, application no. 15, has been published in G. And the activity of the gene product thereof is the activity of AX653549-protein.

Accordingly, in one embodiment, the process of the present invention for producing fumarate in a non-human organism, like a microorganism or a plant or a part thereof, comprises increasing or generating the activity of a gene product with the activity of a gene product conferring the activity of “AX653549-protein”, especially from Oryza sativa or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said AX653549, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AX653549, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AX653549, or a functional equivalent or a homolog thereof as depicted in column 8 of Table II, application no. 15, preferably a homolog or functional equivalent as depicted in column 8 of Table II B, application no. 15, and being depicted in the same respective line as said AX653549, and preferably the activity is increased non-targeted, whereby the respective line disclose in column 7 the fine chemical fumarate.

Accordingly, in one embodiment, the molecule which activity is to be increased in the process of the invention is the gene product with an activity as a “AX653549-protein”, preferably it is the molecule of section (a) or (b) of this paragraph.

In particular, it was observed that in plants, especially in Arabidopsis thaliana, increasing or generating the activity of a gene product non-targeted with the activity of a “AX653549-protein”, preferably being encoded by a gene comprising the nucleic acid sequence SEQ ID NO. 34301, preferably the coding region thereof, conferred the production of or the increase in fumarate compared with the wild type control.

The nucleic acid sequence of AX653549 from Oryza sativa, e.g. as shown in the respective line in column 5 of Table I, application no. 15, has been published in G. And the activity of the gene product thereof is the activity of AX653549-protein.

Accordingly, in one embodiment, the process of the present invention for producing malate in a non-human organism, like a microorganism or a plant or a part thereof, comprises increasing or generating the activity of a gene product with the activity of a gene product conferring the activity of “AX653549-protein”, especially from Oryza sativa or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said AX653549, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AX653549, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AX653549, or a functional equivalent or a homolog thereof as depicted in column 8 of Table II, application no. 15, preferably a homolog or functional equivalent as depicted in column 8 of Table II B, application no. 15, and being depicted in the same respective line as said AX653549, and preferably the activity is increased non-targeted, whereby the respective line disclose in column 7 the fine chemical malate.

Accordingly, in one embodiment, the molecule which activity is to be increased in the process of the invention is the gene product with an activity as a “AX653549-protein”, preferably it is the molecule of section (a) or (b) of this paragraph.

In particular, it was observed that in plants, especially in Arabidopsis thaliana, increasing or generating the activity of a gene product non-targeted with the activity of a “AX653549-protein”, preferably being encoded by a gene comprising the nucleic acid sequence SEQ ID NO. 34301, preferably the coding region thereof, conferred the production of or the increase in malate compared with the wild type control.

The nucleic acid sequence of AY087308 from Arabidopsis thaliana, e.g. as shown in the respective line in column 5 of Table I, application no. 15, has been published in F. And the activity of the gene product thereof is the activity of AY087308-protein.

Accordingly, in one embodiment, the process of the present invention for producing fumarate in a non-human organism, like a microorganism or a plant or a part thereof, comprises increasing or generating the activity of a gene product with the activity of a gene product conferring the activity of “AY087308-protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said AY087308, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AY087308, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AY087308, or a functional equivalent or a homolog thereof as depicted in column 8 of Table II, application no. 15, preferably a homolog or functional equivalent as depicted in column 8 of Table II B, application no. 15, and being depicted in the same respective line as said AY087308, and preferably the activity is increased non-targeted, whereby the respective line disclose in column 7 the fine chemical fumarate.

Accordingly, in one embodiment, the molecule which activity is to be increased in the process of the invention is the gene product with an activity as a “AY087308-protein”, preferably it is the molecule of section (a) or (b) of this paragraph.

In particular, it was observed that in plants, especially in Arabidopsis thaliana, increasing or generating the activity of a gene product non-targeted with the activity of a “AY087308-protein”, preferably being encoded by a gene comprising the nucleic acid sequence SEQ ID NO. 34602, preferably the coding region thereof, conferred the production of or the increase in fumarate compared with the wild type control.

The nucleic acid sequence of AY087308 from Arabidopsis thaliana, e.g. as shown in the respective line in column 5 of Table I, application no. 15, has been published in F. And the activity of the gene product thereof is the activity of AY087308-protein.

Accordingly, in one embodiment, the process of the present invention for producing malate in a non-human organism, like a microorganism or a plant or a part thereof, comprises increasing or generating the activity of a gene product with the activity of a gene product conferring the activity of “AY087308-protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said AY087308, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AY087308, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AY087308, or a functional equivalent or a homolog thereof as depicted in column 8 of Table II, application no. 15, preferably a homolog or functional equivalent as depicted in column 8 of Table II B, application no. 15, and being depicted in the same respective line as said AY087308, and preferably the activity is increased non-targeted, whereby the respective line disclose in column 7 the fine chemical malate.

Accordingly, in one embodiment, the molecule which activity is to be increased in the process of the invention is the gene product with an activity as a “AY087308-protein”, preferably it is the molecule of section (a) or (b) of this paragraph.

In particular, it was observed that in plants, especially in Arabidopsis thaliana, increasing or generating the activity of a gene product non-targeted with the activity of a “AY087308-protein”, preferably being encoded by a gene comprising the nucleic acid sequence SEQ ID NO. 34602, preferably the coding region thereof, conferred the production of or the increase in malate compared with the wild type control.

The nucleic acid sequence of AY087308 from Arabidopsis thaliana, e.g. as shown in the respective line in column 5 of Table I, application no. 15, has been published in F. And the activity of the gene product thereof is the activity of AY087308-protein.

Accordingly, in one embodiment, the process of the present invention for producing succinate in a non-human organism, like a microorganism or a plant or a part thereof, comprises increasing or generating the activity of a gene product with the activity of a gene product conferring the activity of “AY087308-protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said AY087308, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said AY087308, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said AY087308, or a functional equivalent or a homolog thereof as depicted in column 8 of Table II, application no. 15, preferably a homolog or functional equivalent as depicted in column 8 of Table II B, application no. 15, and being depicted in the same respective line as said AY087308, and preferably the activity is increased non-targeted, whereby the respective line disclose in column 7 the fine chemical succinate.

Accordingly, in one embodiment, the molecule which activity is to be increased in the process of the invention is the gene product with an activity as a “AY087308-protein”, preferably it is the molecule of section (a) or (b) of this paragraph.

In particular, it was observed that in plants, especially in Arabidopsis thaliana, increasing or generating the activity of a gene product non-targeted with the activity of a “AY087308-protein”, preferably being encoded by a gene comprising the nucleic acid sequence SEQ ID NO. 34602, preferably the coding region thereof, conferred the production of or the increase in succinate compared with the wild type control.

The nucleic acid sequence of Bab08741 from Arabidopsis thaliana, e.g. as shown in the respective line in column 5 of Table I, application no. 15, has been published in F. And the activity of the gene product thereof is the activity of fumarate hydratase.

Accordingly, in one embodiment, the process of the present invention for producing fumarate in a non-human organism, like a microorganism or a plant or a part thereof, comprises increasing or generating the activity of a gene product with the activity of a gene product conferring the activity of “fumarate hydratase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said Bab08741, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Bab08741, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Bab08741, or a functional equivalent or a homolog thereof as depicted in column 8 of Table II, application no. 15, preferably a homolog or functional equivalent as depicted in column 8 of Table II B, application no. 15, and being depicted in the same respective line as said Bab08741, and preferably the activity is increased non-targeted, whereby the respective line disclose in column 7 the fine chemical fumarate.

Accordingly, in one embodiment, the molecule which activity is to be increased in the process of the invention is the gene product with an activity as a “fumarate hydratase”, preferably it is the molecule of section (a) or (b) of this paragraph.

In particular, it was observed that in plants, especially in Arabidopsis thaliana, increasing or generating the activity of a gene product non-targeted with the activity of a “fumarate hydratase”, preferably being encoded by a gene comprising the nucleic acid sequence SEQ ID NO. 136120, preferably the coding region thereof, conferred the production of or the increase in fumarate compared with the wild type control.

The nucleic acid sequence of B0004 from Escherichia coli, e.g. as shown in the respective line in column 5 of Table I, application no. 15, has been published in B. And the activity of the gene product thereof is the activity of threonine synthase.

Accordingly, in one embodiment, the process of the present invention for producing malate in a non-human organism, like a microorganism or a plant or a part thereof, comprises increasing or generating the activity of a gene product with the activity of a gene product conferring the activity of “threonine synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said B0004, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B0004, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B0004, or a functional equivalent or a homolog thereof as depicted in column 8 of Table II, application no. 15, preferably a homolog or functional equivalent as depicted in column 8 of Table II B, application no. 15, and being depicted in the same respective line as said B0004, and preferably the activity is increased plastidic, whereby the respective line disclose in column 7 the fine chemical malate.

Accordingly, in one embodiment, the molecule which activity is to be increased in the process of the invention is the gene product with an activity as a “threonine synthase”, preferably it is the molecule of section (a) or (b) of this paragraph.

In particular, it was observed that in plants, especially in Arabidopsis thaliana, increasing or generating the activity of a gene product plastidic with the activity of a “threonine synthase”, preferably being encoded by a gene comprising the nucleic acid sequence SEQ ID NO. 34889, preferably the coding region thereof, conferred the production of or the increase in malate compared with the wild type control.

The nucleic acid sequence of B0063 from Escherichia coli, e.g. as shown in the respective line in column 5 of Table I, application no. 15, has been published in B. And the activity of the gene product thereof is the activity of ribulokinase.

Accordingly, in one embodiment, the process of the present invention for producing fumarate in a non-human organism, like a microorganism or a plant or a part thereof, comprises increasing or generating the activity of a gene product with the activity of a gene product conferring the activity of “ribulokinase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said B0063, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B0063, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B0063, or a functional equivalent or a homolog thereof as depicted in column 8 of Table II, application no. 15, preferably a homolog or functional equivalent as depicted in column 8 of Table II B, application no. 15, and being depicted in the same respective line as said B0063, and preferably the activity is increased non-targeted, whereby the respective line disclose in column 7 the fine chemical fumarate.

Accordingly, in one embodiment, the molecule which activity is to be increased in the process of the invention is the gene product with an activity as a “ribulokinase”, preferably it is the molecule of section (a) or (b) of this paragraph.

In particular, it was observed that in plants, especially in Arabidopsis thaliana, increasing or generating the activity of a gene product non-targeted with the activity of a “ribulokinase”, preferably being encoded by a gene comprising the nucleic acid sequence SEQ ID NO. 134299, preferably the coding region thereof, conferred the production of or the increase in fumarate compared with the wild type control.

The nucleic acid sequence of B0063 from Escherichia coli, e.g. as shown in the respective line in column 5 of Table I, application no. 15, has been published in B. And the activity of the gene product thereof is the activity of ribulokinase.

Accordingly, in one embodiment, the process of the present invention for producing succinate in a non-human organism, like a microorganism or a plant or a part thereof, comprises increasing or generating the activity of a gene product with the activity of a gene product conferring the activity of “ribulokinase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said B0063, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B0063, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B0063, or a functional equivalent or a homolog thereof as depicted in column 8 of Table II, application no. 15, preferably a homolog or functional equivalent as depicted in column 8 of Table II B, application no. 15, and being depicted in the same respective line as said B0063, and preferably the activity is increased non-targeted, whereby the respective line disclose in column 7 the fine chemical succinate.

Accordingly, in one embodiment, the molecule which activity is to be increased in the process of the invention is the gene product with an activity as a “ribulokinase”, preferably it is the molecule of section (a) or (b) of this paragraph.

In particular, it was observed that in plants, especially in Arabidopsis thaliana, increasing or generating the activity of a gene product non-targeted with the activity of a “ribulokinase”, preferably being encoded by a gene comprising the nucleic acid sequence SEQ ID NO. 134299, preferably the coding region thereof, conferred the production of or the increase in succinate compared with the wild type control.

The nucleic acid sequence of B0124 from Escherichia coli, e.g. as shown in the respective line in column 5 of Table I, application no. 15, has been published in B. And the activity of the gene product thereof is the activity of glucose dehydrogenase.

Accordingly, in one embodiment, the process of the present invention for producing citrate in a non-human organism, like a microorganism or a plant or a part thereof, comprises increasing or generating the activity of a gene product with the activity of a gene product conferring the activity of “glucose dehydrogenase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical citrate), application no. 15, preferably the coding region thereof, or a homolog or a fragment thereof, and being depicted in the same respective line as said B0124, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B0124, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B0124, or a functional equivalent or a homolog thereof as depicted in column 8 of Table II, application no. 15, preferably a homolog or functional equivalent as depicted in column 8 of Table II B, application no. 15, and being depicted in the same respective line as said B0124, and preferably the activity is increased non-targeted, whereby the respective line disclose in column 7 the fine chemical citrate.

Accordingly, in one embodiment, the molecule which activity is to be increased in the process of the invention is the gene product with an activity as a “glucose dehydrogenase”, preferably it is the molecule of section (a) or (b) of this paragraph.

In particular, it was observed that in plants, especially in Arabidopsis thaliana, increasing or generating the activity of a gene product non-targeted with the activity of a “glucose dehydrogenase”, preferably being encoded by a gene comprising the nucleic acid sequence SEQ ID NO. 35482, preferably the coding region thereof, conferred the production of or the increase in citrate compared with the wild type control.

The nucleic acid sequence of B0221 from Escherichia coli, e.g. as shown in the respective line in column 5 of Table I, application no. 15, has been published in B. And the activity of the gene product thereof is the activity of acyl-CoA dehydrogenase.

Accordingly, in one embodiment, the process of the present invention for producing fumarate in a non-human organism, like a microorganism or a plant or a part thereof, comprises increasing or generating the activity of a gene product with the activity of a gene product conferring the activity of “acyl-CoA dehydrogenase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said B0221, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B0221, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B0221, or a functional equivalent or a homolog thereof as depicted in column 8 of Table II, application no. 15, preferably a homolog or functional equivalent as depicted in column 8 of Table II B, application no. 15, and being depicted in the same respective line as said B0221, and preferably the activity is increased non-targeted, whereby the respective line disclose in column 7 the fine chemical fumarate.

Accordingly, in one embodiment, the molecule which activity is to be increased in the process of the invention is the gene product with an activity as a “acyl-CoA dehydrogenase”, preferably it is the molecule of section (a) or (b) of this paragraph.

In particular, it was observed that in plants, especially in Arabidopsis thaliana, increasing or generating the activity of a gene product non-targeted with the activity of a “acyl-CoA dehydrogenase”, preferably being encoded by a gene comprising the nucleic acid sequence SEQ ID NO. 35590, preferably the coding region thereof, conferred the production of or the increase in fumarate compared with the wild type control.

The nucleic acid sequence of B0312 from Escherichia coli, e.g. as shown in the respective line in column 5 of Table I, application no. 15, has been published in B. And the activity of the gene product thereof is the activity of betaine aldehyde dehydrogenase.

Accordingly, in one embodiment, the process of the present invention for producing citrate in a non-human organism, like a microorganism or a plant or a part thereof, comprises increasing or generating the activity of a gene product with the activity of a gene product conferring the activity of “betaine aldehyde dehydrogenase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical citrate), application no. 15, preferably the coding region thereof, or a homolog or a fragment thereof, and being depicted in the same respective line as said B0312, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B0312, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B0312, or a functional equivalent or a homolog thereof as depicted in column 8 of Table II, application no. 15, preferably a homolog or functional equivalent as depicted in column 8 of Table II B, application no. 15, and being depicted in the same respective line as said B0312, and preferably the activity is increased non-targeted, whereby the respective line disclose in column 7 the fine chemical citrate.

Accordingly, in one embodiment, the molecule which activity is to be increased in the process of the invention is the gene product with an activity as a “betaine aldehyde dehydrogenase”, preferably it is the molecule of section (a) or (b) of this paragraph.

In particular, it was observed that in plants, especially in Arabidopsis thaliana, increasing or generating the activity of a gene product non-targeted with the activity of a “betaine aldehyde dehydrogenase”, preferably being encoded by a gene comprising the nucleic acid sequence SEQ ID NO. 121111, preferably the coding region thereof, conferred the production of or the increase in citrate compared with the wild type control.

The nucleic acid sequence of B0449 from Escherichia coli, e.g. as shown in the respective line in column 5 of Table I, application no. 15, has been published in B. And the activity of the gene product thereof is the activity of ATP-binding component of a transport system.

Accordingly, in one embodiment, the process of the present invention for producing malate in a non-human organism, like a microorganism or a plant or a part thereof, comprises increasing or generating the activity of a gene product with the activity of a gene product conferring the activity of “ATP-binding component of a transport system”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said B0449, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B0449, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B0449, or a functional equivalent or a homolog thereof as depicted in column 8 of Table II, application no. 15, preferably a homolog or functional equivalent as depicted in column 8 of Table II B, application no. 15, and being depicted in the same respective line as said B0449, and preferably the activity is increased non-targeted, whereby the respective line disclose in column 7 the fine chemical malate.

Accordingly, in one embodiment, the molecule which activity is to be increased in the process of the invention is the gene product with an activity as a “ATP-binding component of a transport system”, preferably it is the molecule of section (a) or (b) of this paragraph.

In particular, it was observed that in plants, especially in Arabidopsis thaliana, increasing or generating the activity of a gene product non-targeted with the activity of a “ATP-binding component of a transport system”, preferably being encoded by a gene comprising the nucleic acid sequence SEQ ID NO. 7333, preferably the coding region thereof, conferred the production of or the increase in malate compared with the wild type control.

The nucleic acid sequence of B0486 from Escherichia coli, e.g. as shown in the respective line in column 5 of Table I, application no. 15, has been published in B. And the activity of the gene product thereof is the activity of membrane transport protein.

Accordingly, in one embodiment, the process of the present invention for producing malate in a non-human organism, like a microorganism or a plant or a part thereof, comprises increasing or generating the activity of a gene product with the activity of a gene product conferring the activity of “membrane transport protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said B0486, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B0486, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B0486, or a functional equivalent or a homolog thereof as depicted in column 8 of Table II, application no. 15, preferably a homolog or functional equivalent as depicted in column 8 of Table II B, application no. 15, and being depicted in the same respective line as said B0486, and preferably the activity is increased non-targeted, whereby the respective line disclose in column 7 the fine chemical malate.

Accordingly, in one embodiment, the molecule which activity is to be increased in the process of the invention is the gene product with an activity as a “membrane transport protein”, preferably it is the molecule of section (a) or (b) of this paragraph.

In particular, it was observed that in plants, especially in Arabidopsis thaliana, increasing or generating the activity of a gene product non-targeted with the activity of a “membrane transport protein”, preferably being encoded by a gene comprising the nucleic acid sequence SEQ ID NO. 7686, preferably the coding region thereof, conferred the production of or the increase in malate compared with the wild type control.

The nucleic acid sequence of B0486 from Escherichia coli, e.g. as shown in the respective line in column 5 of Table I, application no. 15, has been published in B. And the activity of the gene product thereof is the activity of membrane transport protein.

Accordingly, in one embodiment, the process of the present invention for producing succinate in a non-human organism, like a microorganism or a plant or a part thereof, comprises increasing or generating the activity of a gene product with the activity of a gene product conferring the activity of “membrane transport protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said B0486, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B0486, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B0486, or a functional equivalent or a homolog thereof as depicted in column 8 of Table II, application no. 15, preferably a homolog or functional equivalent as depicted in column 8 of Table II B, application no. 15, and being depicted in the same respective line as said B0486, and preferably the activity is increased non-targeted, whereby the respective line disclose in column 7 the fine chemical succinate.

Accordingly, in one embodiment, the molecule which activity is to be increased in the process of the invention is the gene product with an activity as a “membrane transport protein”, preferably it is the molecule of section (a) or (b) of this paragraph.

In particular, it was observed that in plants, especially in Arabidopsis thaliana, increasing or generating the activity of a gene product non-targeted with the activity of a “membrane transport protein”, preferably being encoded by a gene comprising the nucleic acid sequence SEQ ID NO. 7686, preferably the coding region thereof, conferred the production of or the increase in succinate compared with the wild type control.

The nucleic acid sequence of B0514 from Escherichia coli, e.g. as shown in the respective line in column 5 of Table I, application no. 15, has been published in B. And the activity of the gene product thereof is the activity of glycerate kinase.

Accordingly, in one embodiment, the process of the present invention for producing citrate in a non-human organism, like a microorganism or a plant or a part thereof, comprises increasing or generating the activity of a gene product with the activity of a gene product conferring the activity of “glycerate kinase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical citrate), application no. 15, preferably the coding region thereof, or a homolog or a fragment thereof, and being depicted in the same respective line as said B0514, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B0514, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B0514, or a functional equivalent or a homolog thereof as depicted in column 8 of Table II, application no. 15, preferably a homolog or functional equivalent as depicted in column 8 of Table II B, application no. 15, and being depicted in the same respective line as said B0514, and preferably the activity is increased non-targeted, whereby the respective line disclose in column 7 the fine chemical citrate.

Accordingly, in one embodiment, the molecule which activity is to be increased in the process of the invention is the gene product with an activity as a “glycerate kinase”, preferably it is the molecule of section (a) or (b) of this paragraph.

In particular, it was observed that in plants, especially in Arabidopsis thaliana, increasing or generating the activity of a gene product non-targeted with the activity of a “glycerate kinase”, preferably being encoded by a gene comprising the nucleic acid sequence SEQ ID NO. 134402, preferably the coding region thereof, conferred the production of or the increase in citrate compared with the wild type control.

The nucleic acid sequence of B0593 from Escherichia coli, e.g. as shown in the respective line in column 5 of Table I, application no. 15, has been published in B. And the activity of the gene product thereof is the activity of isochorismate synthase.

Accordingly, in one embodiment, the process of the present invention for producing fumarate in a non-human organism, like a microorganism or a plant or a part thereof, comprises increasing or generating the activity of a gene product with the activity of a gene product conferring the activity of “isochorismate synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said B0593, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B0593, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B0593, or a functional equivalent or a homolog thereof as depicted in column 8 of Table II, application no. 15, preferably a homolog or functional equivalent as depicted in column 8 of Table II B, application no. 15, and being depicted in the same respective line as said B0593, and preferably the activity is increased plastidic, whereby the respective line disclose in column 7 the fine chemical fumarate.

Accordingly, in one embodiment, the molecule which activity is to be increased in the process of the invention is the gene product with an activity as a “isochorismate synthase”, preferably it is the molecule of section (a) or (b) of this paragraph.

In particular, it was observed that in plants, especially in Arabidopsis thaliana, increasing or generating the activity of a gene product plastidic with the activity of a “isochorismate synthase”, preferably being encoded by a gene comprising the nucleic acid sequence SEQ ID NO. 35967, preferably the coding region thereof, conferred the production of or the increase in fumarate compared with the wild type control.

The nucleic acid sequence of B0593 from Escherichia coli, e.g. as shown in the respective line in column 5 of Table I, application no. 15, has been published in B. And the activity of the gene product thereof is the activity of isochorismate synthase.

Accordingly, in one embodiment, the process of the present invention for producing malate in a non-human organism, like a microorganism or a plant or a part thereof, comprises increasing or generating the activity of a gene product with the activity of a gene product conferring the activity of “isochorismate synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said B0593, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B0593, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B0593, or a functional equivalent or a homolog thereof as depicted in column 8 of Table II, application no. 15, preferably a homolog or functional equivalent as depicted in column 8 of Table II B, application no. 15, and being depicted in the same respective line as said B0593, and preferably the activity is increased plastidic, whereby the respective line disclose in column 7 the fine chemical malate.

Accordingly, in one embodiment, the molecule which activity is to be increased in the process of the invention is the gene product with an activity as a “isochorismate synthase”, preferably it is the molecule of section (a) or (b) of this paragraph.

In particular, it was observed that in plants, especially in Arabidopsis thaliana, increasing or generating the activity of a gene product plastidic with the activity of a “isochorismate synthase”, preferably being encoded by a gene comprising the nucleic acid sequence SEQ ID NO. 35967, preferably the coding region thereof, conferred the production of or the increase in malate compared with the wild type control.

The nucleic acid sequence of B0752 from Escherichia coli, e.g. as shown in the respective line in column 5 of Table I, application no. 15, has been published in B. And the activity of the gene product thereof is the activity of zinc transporter.

Accordingly, in one embodiment, the process of the present invention for producing fumarate in a non-human organism, like a microorganism or a plant or a part thereof, comprises increasing or generating the activity of a gene product with the activity of a gene product conferring the activity of “zinc transporter”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said B0752, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B0752, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B0752, or a functional equivalent or a homolog thereof as depicted in column 8 of Table II, application no. 15, preferably a homolog or functional equivalent as depicted in column 8 of Table II B, application no. 15, and being depicted in the same respective line as said B0752, and preferably the activity is increased non-targeted, whereby the respective line disclose in column 7 the fine chemical fumarate.

Accordingly, in one embodiment, the molecule which activity is to be increased in the process of the invention is the gene product with an activity as a “zinc transporter”, preferably it is the molecule of section (a) or (b) of this paragraph.

In particular, it was observed that in plants, especially in Arabidopsis thaliana, increasing or generating the activity of a gene product non-targeted with the activity of a “zinc transporter”, preferably being encoded by a gene comprising the nucleic acid sequence SEQ ID NO. 36114, preferably the coding region thereof, conferred the production of or the increase in fumarate compared with the wild type control.

The nucleic acid sequence of B0801 from Escherichia coli, e.g. as shown in the respective line in column 5 of Table I, application no. 15, has been published in B. And the activity of the gene product thereof is the activity of b0801-protein.

Accordingly, in one embodiment, the process of the present invention for producing succinate in a non-human organism, like a microorganism or a plant or a part thereof, comprises increasing or generating the activity of a gene product with the activity of a gene product conferring the activity of “b0801-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said B0801, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B0801, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B0801, or a functional equivalent or a homolog thereof as depicted in column 8 of Table II, application no. 15, preferably a homolog or functional equivalent as depicted in column 8 of Table II B, application no. 15, and being depicted in the same respective line as said B0801, and preferably the activity is increased non-targeted, whereby the respective line disclose in column 7 the fine chemical succinate.

Accordingly, in one embodiment, the molecule which activity is to be increased in the process of the invention is the gene product with an activity as a “b0801-protein”, preferably it is the molecule of section (a) or (b) of this paragraph.

In particular, it was observed that in plants, especially in Arabidopsis thaliana, increasing or generating the activity of a gene product non-targeted with the activity of a “b0801-protein”, preferably being encoded by a gene comprising the nucleic acid sequence SEQ ID NO. 75807, preferably the coding region thereof, conferred the production of or the increase in succinate compared with the wild type control.

The nucleic acid sequence of B0885 from Escherichia coli, e.g. as shown in the respective line in column 5 of Table I, application no. 15, has been published in B. And the activity of the gene product thereof is the activity of leucyl/phenylalanyl-tRNA-protein transferase.

Accordingly, in one embodiment, the process of the present invention for producing malate in a non-human organism, like a microorganism or a plant or a part thereof, comprises increasing or generating the activity of a gene product with the activity of a gene product conferring the activity of “leucyl/phenylalanyl-tRNA-protein transferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said B0885, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B0885, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B0885, or a functional equivalent or a homolog thereof as depicted in column 8 of Table II, application no. 15, preferably a homolog or functional equivalent as depicted in column 8 of Table II B, application no. 15, and being depicted in the same respective line as said B0885, and preferably the activity is increased non-targeted, whereby the respective line disclose in column 7 the fine chemical malate.

Accordingly, in one embodiment, the molecule which activity is to be increased in the process of the invention is the gene product with an activity as a “leucyl/phenylalanyl-tRNA-protein transferase”, preferably it is the molecule of section (a) or (b) of this paragraph.

In particular, it was observed that in plants, especially in Arabidopsis thaliana, increasing or generating the activity of a gene product non-targeted with the activity of a “leucyl/phenylalanyl-tRNA-protein transferase”, preferably being encoded by a gene comprising the nucleic acid sequence SEQ ID NO. 36489, preferably the coding region thereof, conferred the production of or the increase in malate compared with the wild type control.

The nucleic acid sequence of B0963 from Escherichia coli, e.g. as shown in the respective line in column 5 of Table I, application no. 15, has been published in B. And the activity of the gene product thereof is the activity of methylglyoxal synthase.

Accordingly, in one embodiment, the process of the present invention for producing vitamin c and/or threonic acid in a non-human organism, like a microorganism or a plant or a part thereof, comprises increasing or generating the activity of a gene product with the activity of a gene product conferring the activity of “methylglyoxal synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical vitamin c and/or threonic acid), application no. 15, preferably the coding region thereof, or a homolog or a fragment thereof, and being depicted in the same respective line as said B0963, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B0963, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B0963, or a functional equivalent or a homolog thereof as depicted in column 8 of Table II, application no. 15, preferably a homolog or functional equivalent as depicted in column 8 of Table II B, application no. 15, and being depicted in the same respective line as said B0963, and preferably the activity is increased non-targeted, whereby the respective line disclose in column 7 the fine chemical vitamin c and/or threonic acid.

Accordingly, in one embodiment, the molecule which activity is to be increased in the process of the invention is the gene product with an activity as a “methylglyoxal synthase”, preferably it is the molecule of section (a) or (b) of this paragraph.

In particular, it was observed that in plants, especially in Arabidopsis thaliana, increasing or generating the activity of a gene product non-targeted with the activity of a “methylglyoxal synthase”, preferably being encoded by a gene comprising the nucleic acid sequence SEQ ID NO. 36670, preferably the coding region thereof, conferred the production of or the increase in vitamin c and/or threonic acid compared with the wild type control.

The nucleic acid sequence of B1003 from Escherichia coli, e.g. as shown in the respective line in column 5 of Table I, application no. 15, has been published in B. And the activity of the gene product thereof is the activity of b1003-protein.

Accordingly, in one embodiment, the process of the present invention for producing malate in a non-human organism, like a microorganism or a plant or a part thereof, comprises increasing or generating the activity of a gene product with the activity of a gene product conferring the activity of “b1003-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said B1003, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B1003, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B1003, or a functional equivalent or a homolog thereof as depicted in column 8 of Table II, application no. 15, preferably a homolog or functional equivalent as depicted in column 8 of Table II B, application no. 15, and being depicted in the same respective line as said B1003, and preferably the activity is increased non-targeted, whereby the respective line disclose in column 7 the fine chemical malate.

Accordingly, in one embodiment, the molecule which activity is to be increased in the process of the invention is the gene product with an activity as a “b1003-protein”, preferably it is the molecule of section (a) or (b) of this paragraph.

In particular, it was observed that in plants, especially in Arabidopsis thaliana, increasing or generating the activity of a gene product non-targeted with the activity of a “b1003-protein”, preferably being encoded by a gene comprising the nucleic acid sequence SEQ ID NO. 7941, preferably the coding region thereof, conferred the production of or the increase in malate compared with the wild type control.

The nucleic acid sequence of B1255 from Escherichia coli, e.g. as shown in the respective line in column 5 of Table I, application no. 15, has been published in B. And the activity of the gene product thereof is the activity of membrane protein.

Accordingly, in one embodiment, the process of the present invention for producing malate in a non-human organism, like a microorganism or a plant or a part thereof, comprises increasing or generating the activity of a gene product with the activity of a gene product conferring the activity of “membrane protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said B1255, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B1255, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B1255, or a functional equivalent or a homolog thereof as depicted in column 8 of Table II, application no. 15, preferably a homolog or functional equivalent as depicted in column 8 of Table II B, application no. 15, and being depicted in the same respective line as said B1255, and preferably the activity is increased non-targeted, whereby the respective line disclose in column 7 the fine chemical malate.

Accordingly, in one embodiment, the molecule which activity is to be increased in the process of the invention is the gene product with an activity as a “membrane protein”, preferably it is the molecule of section (a) or (b) of this paragraph.

In particular, it was observed that in plants, especially in Arabidopsis thaliana, increasing or generating the activity of a gene product non-targeted with the activity of a “membrane protein”, preferably being encoded by a gene comprising the nucleic acid sequence SEQ ID NO. 37483, preferably the coding region thereof, conferred the production of or the increase in malate compared with the wild type control.

The nucleic acid sequence of B1285 from Escherichia coli, e.g. as shown in the respective line in column 5 of Table I, application no. 15, has been published in B. And the activity of the gene product thereof is the activity of b1285-protein.

Accordingly, in one embodiment, the process of the present invention for producing succinate in a non-human organism, like a microorganism or a plant or a part thereof, comprises increasing or generating the activity of a gene product with the activity of a gene product conferring the activity of “b1285-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said B1285, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B1285, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B1285, or a functional equivalent or a homolog thereof as depicted in column 8 of Table II, application no. 15, preferably a homolog or functional equivalent as depicted in column 8 of Table II B, application no. 15, and being depicted in the same respective line as said B1285, and preferably the activity is increased non-targeted, whereby the respective line disclose in column 7 the fine chemical succinate.

Accordingly, in one embodiment, the molecule which activity is to be increased in the process of the invention is the gene product with an activity as a “b1285-protein”, preferably it is the molecule of section (a) or (b) of this paragraph.

In particular, it was observed that in plants, especially in Arabidopsis thaliana, increasing or generating the activity of a gene product non-targeted with the activity of a “b1285-protein”, preferably being encoded by a gene comprising the nucleic acid sequence SEQ ID NO. 134655, preferably the coding region thereof, conferred the production of or the increase in succinate compared with the wild type control.

The nucleic acid sequence of B1390 from Escherichia coli, e.g. as shown in the respective line in column 5 of Table I, application no. 15, has been published in B. And the activity of the gene product thereof is the activity of phenylacetic acid degradation protein.

Accordingly, in one embodiment, the process of the present invention for producing malate in a non-human organism, like a microorganism or a plant or a part thereof, comprises increasing or generating the activity of a gene product with the activity of a gene product conferring the activity of “phenylacetic acid degradation protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said B1390, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B1390, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B1390, or a functional equivalent or a homolog thereof as depicted in column 8 of Table II, application no. 15, preferably a homolog or functional equivalent as depicted in column 8 of Table II B, application no. 15, and being depicted in the same respective line as said B1390, and preferably the activity is increased non-targeted, whereby the respective line disclose in column 7 the fine chemical malate.

Accordingly, in one embodiment, the molecule which activity is to be increased in the process of the invention is the gene product with an activity as a “phenylacetic acid degradation protein”, preferably it is the molecule of section (a) or (b) of this paragraph.

In particular, it was observed that in plants, especially in Arabidopsis thaliana, increasing or generating the activity of a gene product non-targeted with the activity of a “phenylacetic acid degradation protein”, preferably being encoded by a gene comprising the nucleic acid sequence SEQ ID NO. 134697, preferably the coding region thereof, conferred the production of or the increase in malate compared with the wild type control.

The nucleic acid sequence of B1425 from Escherichia coli, e.g. as shown in the respective line in column 5 of Table I, application no. 15, has been published in B. And the activity of the gene product thereof is the activity of b1425-protein.

Accordingly, in one embodiment, the process of the present invention for producing malate in a non-human organism, like a microorganism or a plant or a part thereof, comprises increasing or generating the activity of a gene product with the activity of a gene product conferring the activity of “b1425-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said B1425, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B1425, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B1425, or a functional equivalent or a homolog thereof as depicted in column 8 of Table II, application no. 15, preferably a homolog or functional equivalent as depicted in column 8 of Table II B, application no. 15, and being depicted in the same respective line as said B1425, and preferably the activity is increased non-targeted, whereby the respective line disclose in column 7 the fine chemical malate.

Accordingly, in one embodiment, the molecule which activity is to be increased in the process of the invention is the gene product with an activity as a “b1425-protein”, preferably it is the molecule of section (a) or (b) of this paragraph.

In particular, it was observed that in plants, especially in Arabidopsis thaliana, increasing or generating the activity of a gene product non-targeted with the activity of a “b1425-protein”, preferably being encoded by a gene comprising the nucleic acid sequence SEQ ID NO. 134756, preferably the coding region thereof, conferred the production of or the increase in malate compared with the wild type control.

The nucleic acid sequence of B1431 from Escherichia coli, e.g. as shown in the respective line in column 5 of Table I, application no. 15, has been published in B. And the activity of the gene product thereof is the activity of lipoprotein precursor.

Accordingly, in one embodiment, the process of the present invention for producing succinate in a non-human organism, like a microorganism or a plant or a part thereof, comprises increasing or generating the activity of a gene product with the activity of a gene product conferring the activity of “lipoprotein precursor”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said B1431, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B1431, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B1431, or a functional equivalent or a homolog thereof as depicted in column 8 of Table II, application no. 15, preferably a homolog or functional equivalent as depicted in column 8 of Table II B, application no. 15, and being depicted in the same respective line as said B1431, and preferably the activity is increased plastidic, whereby the respective line disclose in column 7 the fine chemical succinate.

Accordingly, in one embodiment, the molecule which activity is to be increased in the process of the invention is the gene product with an activity as a “lipoprotein precursor”, preferably it is the molecule of section (a) or (b) of this paragraph.

In particular, it was observed that in plants, especially in Arabidopsis thaliana, increasing or generating the activity of a gene product plastidic with the activity of a “lipoprotein precursor”, preferably being encoded by a gene comprising the nucleic acid sequence SEQ ID NO. 38266, preferably the coding region thereof, conferred the production of or the increase in succinate compared with the wild type control.

The nucleic acid sequence of B1445 from Escherichia coli, e.g. as shown in the respective line in column 5 of Table I, application no. 15, has been published in B. And the activity of the gene product thereof is the activity of b1445-protein.

Accordingly, in one embodiment, the process of the present invention for producing citramalate in a non-human organism, like a microorganism or a plant or a part thereof, comprises increasing or generating the activity of a gene product with the activity of a gene product conferring the activity of “b1445-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said B1445, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B1445, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B1445, or a functional equivalent or a homolog thereof as depicted in column 8 of Table II, application no. 15, preferably a homolog or functional equivalent as depicted in column 8 of Table II B, application no. 15, and being depicted in the same respective line as said B1445, and preferably the activity is increased non-targeted, whereby the respective line disclose in column 7 the fine chemical citramalate.

Accordingly, in one embodiment, the molecule which activity is to be increased in the process of the invention is the gene product with an activity as a “b1445-protein”, preferably it is the molecule of section (a) or (b) of this paragraph.

In particular, it was observed that in plants, especially in Arabidopsis thaliana, increasing or generating the activity of a gene product non-targeted with the activity of a “b1445-protein”, preferably being encoded by a gene comprising the nucleic acid sequence SEQ ID NO. 38289, preferably the coding region thereof, conferred the production of or the increase in citramalate compared with the wild type control.

The nucleic acid sequence of B1522 from Escherichia coli, e.g. as shown in the respective line in column 5 of Table I, application no. 15, has been published in B. And the activity of the gene product thereof is the activity of b1522-protein.

Accordingly, in one embodiment, the process of the present invention for producing citramalate in a non-human organism, like a microorganism or a plant or a part thereof, comprises increasing or generating the activity of a gene product with the activity of a gene product conferring the activity of “b1522-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said B1522, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B1522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B1522, or a functional equivalent or a homolog thereof as depicted in column 8 of Table II, application no. 15, preferably a homolog or functional equivalent as depicted in column 8 of Table II B, application no. 15, and being depicted in the same respective line as said B1522, and preferably the activity is increased non-targeted, whereby the respective line disclose in column 7 the fine chemical citramalate.

Accordingly, in one embodiment, the molecule which activity is to be increased in the process of the invention is the gene product with an activity as a “b1522-protein”, preferably it is the molecule of section (a) or (b) of this paragraph.

In particular, it was observed that in plants, especially in Arabidopsis thaliana, increasing or generating the activity of a gene product non-targeted with the activity of a “b1522-protein”, preferably being encoded by a gene comprising the nucleic acid sequence SEQ ID NO. 7947, preferably the coding region thereof, conferred the production of or the increase in citramalate compared with the wild type control.

The nucleic acid sequence of B1522 from Escherichia coli, e.g. as shown in the respective line in column 5 of Table I, application no. 15, has been published in B. And the activity of the gene product thereof is the activity of b1522-protein.

Accordingly, in one embodiment, the process of the present invention for producing malate in a non-human organism, like a microorganism or a plant or a part thereof, comprises increasing or generating the activity of a gene product with the activity of a gene product conferring the activity of “b1522-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said B1522, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B1522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B1522, or a functional equivalent or a homolog thereof as depicted in column 8 of Table II, application no. 15, preferably a homolog or functional equivalent as depicted in column 8 of Table II B, application no. 15, and being depicted in the same respective line as said B1522, and preferably the activity is increased non-targeted, whereby the respective line disclose in column 7 the fine chemical malate.

Accordingly, in one embodiment, the molecule which activity is to be increased in the process of the invention is the gene product with an activity as a “b1522-protein”, preferably it is the molecule of section (a) or (b) of this paragraph.

In particular, it was observed that in plants, especially in Arabidopsis thaliana, increasing or generating the activity of a gene product non-targeted with the activity of a “b1522-protein”, preferably being encoded by a gene comprising the nucleic acid sequence SEQ ID NO. 7947, preferably the coding region thereof, conferred the production of or the increase in malate compared with the wild type control.

The nucleic acid sequence of B1589 from Escherichia coli, e.g. as shown in the respective line in column 5 of Table I, application no. 15, has been published in B. And the activity of the gene product thereof is the activity of Fe—S subunit of oxidoreductase.

Accordingly, in one embodiment, the process of the present invention for producing fumarate in a non-human organism, like a microorganism or a plant or a part thereof, comprises increasing or generating the activity of a gene product with the activity of a gene product conferring the activity of “Fe—S subunit of oxidoreductase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said B1589, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B1589, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B1589, or a functional equivalent or a homolog thereof as depicted in column 8 of Table II, application no. 15, preferably a homolog or functional equivalent as depicted in column 8 of Table II B, application no. 15, and being depicted in the same respective line as said B1589, and preferably the activity is increased non-targeted, whereby the respective line disclose in column 7 the fine chemical fumarate.

Accordingly, in one embodiment, the molecule which activity is to be increased in the process of the invention is the gene product with an activity as a “Fe—S subunit of oxidoreductase”, preferably it is the molecule of section (a) or (b) of this paragraph.

In particular, it was observed that in plants, especially in Arabidopsis thaliana, increasing or generating the activity of a gene product non-targeted with the activity of a “Fe—S subunit of oxidoreductase”, preferably being encoded by a gene comprising the nucleic acid sequence SEQ ID NO. 78154, preferably the coding region thereof, conferred the production of or the increase in fumarate compared with the wild type control.

The nucleic acid sequence of B1611 from Escherichia coli, e.g. as shown in the respective line in column 5 of Table I, application no. 15, has been published in B. And the activity of the gene product thereof is the activity of fumarate hydratase.

Accordingly, in one embodiment, the process of the present invention for producing fumarate in a non-human organism, like a microorganism or a plant or a part thereof, comprises increasing or generating the activity of a gene product with the activity of a gene product conferring the activity of “fumarate hydratase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said B1611, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B1611, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B1611, or a functional equivalent or a homolog thereof as depicted in column 8 of Table II, application no. 15, preferably a homolog or functional equivalent as depicted in column 8 of Table II B, application no. 15, and being depicted in the same respective line as said B1611, and preferably the activity is increased plastidic, whereby the respective line disclose in column 7 the fine chemical fumarate.

Accordingly, in one embodiment, the molecule which activity is to be increased in the process of the invention is the gene product with an activity as a “fumarate hydratase”, preferably it is the molecule of section (a) or (b) of this paragraph.

In particular, it was observed that in plants, especially in Arabidopsis thaliana, increasing or generating the activity of a gene product plastidic with the activity of a “fumarate hydratase”, preferably being encoded by a gene comprising the nucleic acid sequence SEQ ID NO. 115064, preferably the coding region thereof, conferred the production of or the increase in fumarate compared with the wild type control.

The nucleic acid sequence of B1627 from Escherichia coli, e.g. as shown in the respective line in column 5 of Table I, application no. 15, has been published in B. And the activity of the gene product thereof is the activity of electron transport complex protein.

Accordingly, in one embodiment, the process of the present invention for producing succinate in a non-human organism, like a microorganism or a plant or a part thereof, comprises increasing or generating the activity of a gene product with the activity of a gene product conferring the activity of “electron transport complex protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said B1627, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B1627, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B1627, or a functional equivalent or a homolog thereof as depicted in column 8 of Table II, application no. 15, preferably a homolog or functional equivalent as depicted in column 8 of Table II B, application no. 15, and being depicted in the same respective line as said B1627, and preferably the activity is increased non-targeted, whereby the respective line disclose in column 7 the fine chemical succinate.

Accordingly, in one embodiment, the molecule which activity is to be increased in the process of the invention is the gene product with an activity as a “electron transport complex protein”, preferably it is the molecule of section (a) or (b) of this paragraph.

In particular, it was observed that in plants, especially in Arabidopsis thaliana, increasing or generating the activity of a gene product non-targeted with the activity of a “electron transport complex protein”, preferably being encoded by a gene comprising the nucleic acid sequence SEQ ID NO. 38345, preferably the coding region thereof, conferred the production of or the increase in succinate compared with the wild type control.

The nucleic acid sequence of B1651 from Escherichia coli, e.g. as shown in the respective line in column 5 of Table I, application no. 15, has been published in B. And the activity of the gene product thereof is the activity of methylglyoxalase.

Accordingly, in one embodiment, the process of the present invention for producing succinate in a non-human organism, like a microorganism or a plant or a part thereof, comprises increasing or generating the activity of a gene product with the activity of a gene product conferring the activity of “methylglyoxalase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said B1651, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B1651, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B1651, or a functional equivalent or a homolog thereof as depicted in column 8 of Table II, application no. 15, preferably a homolog or functional equivalent as depicted in column 8 of Table II B, application no. 15, and being depicted in the same respective line as said B1651, and preferably the activity is increased non-targeted, whereby the respective line disclose in column 7 the fine chemical succinate.

Accordingly, in one embodiment, the molecule which activity is to be increased in the process of the invention is the gene product with an activity as a “methylglyoxalase”, preferably it is the molecule of section (a) or (b) of this paragraph.

In particular, it was observed that in plants, especially in Arabidopsis thaliana, increasing or generating the activity of a gene product non-targeted with the activity of a “methylglyoxalase”, preferably being encoded by a gene comprising the nucleic acid sequence SEQ ID NO. 78265, preferably the coding region thereof, conferred the production of or the increase in succinate compared with the wild type control.

The nucleic acid sequence of B1670 from Escherichia coli, e.g. as shown in the respective line in column 5 of Table I, application no. 15, has been published in B. And the activity of the gene product thereof is the activity of b1670-protein.

Accordingly, in one embodiment, the process of the present invention for producing citramalate in a non-human organism, like a microorganism or a plant or a part thereof, comprises increasing or generating the activity of a gene product with the activity of a gene product conferring the activity of “b1670-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said B1670, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B1670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B1670, or a functional equivalent or a homolog thereof as depicted in column 8 of Table II, application no. 15, preferably a homolog or functional equivalent as depicted in column 8 of Table II B, application no. 15, and being depicted in the same respective line as said B1670, and preferably the activity is increased non-targeted, whereby the respective line disclose in column 7 the fine chemical citramalate.

Accordingly, in one embodiment, the molecule which activity is to be increased in the process of the invention is the gene product with an activity as a “b1670-protein”, preferably it is the molecule of section (a) or (b) of this paragraph.

In particular, it was observed that in plants, especially in Arabidopsis thaliana, increasing or generating the activity of a gene product non-targeted with the activity of a “b1670-protein”, preferably being encoded by a gene comprising the nucleic acid sequence SEQ ID NO. 78753, preferably the coding region thereof, conferred the production of or the increase in citramalate compared with the wild type control.

The nucleic acid sequence of B1670 from Escherichia coli, e.g. as shown in the respective line in column 5 of Table I, application no. 15, has been published in B. And the activity of the gene product thereof is the activity of b1670-protein.

Accordingly, in one embodiment, the process of the present invention for producing malate in a non-human organism, like a microorganism or a plant or a part thereof, comprises increasing or generating the activity of a gene product with the activity of a gene product conferring the activity of “b1670-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said B1670, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B1670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B1670, or a functional equivalent or a homolog thereof as depicted in column 8 of Table II, application no. 15, preferably a homolog or functional equivalent as depicted in column 8 of Table II B, application no. 15, and being depicted in the same respective line as said B1670, and preferably the activity is increased non-targeted, whereby the respective line disclose in column 7 the fine chemical malate.

Accordingly, in one embodiment, the molecule which activity is to be increased in the process of the invention is the gene product with an activity as a “b1670-protein”, preferably it is the molecule of section (a) or (b) of this paragraph.

In particular, it was observed that in plants, especially in Arabidopsis thaliana, increasing or generating the activity of a gene product non-targeted with the activity of a “b1670-protein”, preferably being encoded by a gene comprising the nucleic acid sequence SEQ ID NO. 78753, preferably the coding region thereof, conferred the production of or the increase in malate compared with the wild type control.

The nucleic acid sequence of B1672 from Escherichia coli, e.g. as shown in the respective line in column 5 of Table I, application no. 15, has been published in B. And the activity of the gene product thereof is the activity of b1672-protein.

Accordingly, in one embodiment, the process of the present invention for producing citramalate in a non-human organism, like a microorganism or a plant or a part thereof, comprises increasing or generating the activity of a gene product with the activity of a gene product conferring the activity of “b1672-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said B1672, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B1672, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B1672, or a functional equivalent or a homolog thereof as depicted in column 8 of Table II, application no. 15, preferably a homolog or functional equivalent as depicted in column 8 of Table II B, application no. 15, and being depicted in the same respective line as said B1672, and preferably the activity is increased non-targeted, whereby the respective line disclose in column 7 the fine chemical citramalate.

Accordingly, in one embodiment, the molecule which activity is to be increased in the process of the invention is the gene product with an activity as a “b1672-protein”, preferably it is the molecule of section (a) or (b) of this paragraph.

In particular, it was observed that in plants, especially in Arabidopsis thaliana, increasing or generating the activity of a gene product non-targeted with the activity of a “b1672-protein”, preferably being encoded by a gene comprising the nucleic acid sequence SEQ ID NO. 78771, preferably the coding region thereof, conferred the production of or the increase in citramalate compared with the wild type control.

The nucleic acid sequence of B1672 from Escherichia coli, e.g. as shown in the respective line in column 5 of Table I, application no. 15, has been published in B. And the activity of the gene product thereof is the activity of b1672-protein.

Accordingly, in one embodiment, the process of the present invention for producing malate in a non-human organism, like a microorganism or a plant or a part thereof, comprises increasing or generating the activity of a gene product with the activity of a gene product conferring the activity of “b1672-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said B1672, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B1672, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B1672, or a functional equivalent or a homolog thereof as depicted in column 8 of Table II, application no. 15, preferably a homolog or functional equivalent as depicted in column 8 of Table II B, application no. 15, and being depicted in the same respective line as said B1672, and preferably the activity is increased non-targeted, whereby the respective line disclose in column 7 the fine chemical malate.

Accordingly, in one embodiment, the molecule which activity is to be increased in the process of the invention is the gene product with an activity as a “b1672-protein”, preferably it is the molecule of section (a) or (b) of this paragraph.

In particular, it was observed that in plants, especially in Arabidopsis thaliana, increasing or generating the activity of a gene product non-targeted with the activity of a “b1672-protein”, preferably being encoded by a gene comprising the nucleic acid sequence SEQ ID NO. 78771, preferably the coding region thereof, conferred the production of or the increase in malate compared with the wild type control.

The nucleic acid sequence of B1672 from Escherichia coli, e.g. as shown in the respective line in column 5 of Table I, application no. 15, has been published in B. And the activity of the gene product thereof is the activity of b1672-protein.

Accordingly, in one embodiment, the process of the present invention for producing vitamin c and/or threonic acid in a non-human organism, like a microorganism or a plant or a part thereof, comprises increasing or generating the activity of a gene product with the activity of a gene product conferring the activity of “b1672-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical vitamin c and/or threonic acid), application no. 15, preferably the coding region thereof, or a homolog or a fragment thereof, and being depicted in the same respective line as said B1672, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B1672, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B1672, or a functional equivalent or a homolog thereof as depicted in column 8 of Table II, application no. 15, preferably a homolog or functional equivalent as depicted in column 8 of Table II B, application no. 15, and being depicted in the same respective line as said B1672, and preferably the activity is increased non-targeted, whereby the respective line disclose in column 7 the fine chemical vitamin c and/or threonic acid.

Accordingly, in one embodiment, the molecule which activity is to be increased in the process of the invention is the gene product with an activity as a “b1672-protein”, preferably it is the molecule of section (a) or (b) of this paragraph.

In particular, it was observed that in plants, especially in Arabidopsis thaliana, increasing or generating the activity of a gene product non-targeted with the activity of a “b1672-protein”, preferably being encoded by a gene comprising the nucleic acid sequence SEQ ID NO. 78771, preferably the coding region thereof, conferred the production of or the increase in vitamin c and/or threonic acid compared with the wild type control.

The nucleic acid sequence of B1742 from Escherichia coli, e.g. as shown in the respective line in column 5 of Table I, application no. 15, has been published in B. And the activity of the gene product thereof is the activity of b1742-protein.

Accordingly, in one embodiment, the process of the present invention for producing malate in a non-human organism, like a microorganism or a plant or a part thereof, comprises increasing or generating the activity of a gene product with the activity of a gene product conferring the activity of “b1742-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said B1742, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B1742, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B1742, or a functional equivalent or a homolog thereof as depicted in column 8 of Table II, application no. 15, preferably a homolog or functional equivalent as depicted in column 8 of Table II B, application no. 15, and being depicted in the same respective line as said B1742, and preferably the activity is increased non-targeted, whereby the respective line disclose in column 7 the fine chemical malate.

Accordingly, in one embodiment, the molecule which activity is to be increased in the process of the invention is the gene product with an activity as a “b1742-protein”, preferably it is the molecule of section (a) or (b) of this paragraph.

In particular, it was observed that in plants, especially in Arabidopsis thaliana, increasing or generating the activity of a gene product non-targeted with the activity of a “b1742-protein”, preferably being encoded by a gene comprising the nucleic acid sequence SEQ ID NO. 134762, preferably the coding region thereof, conferred the production of or the increase in malate compared with the wild type control.

The nucleic acid sequence of B1742 from Escherichia coli, e.g. as shown in the respective line in column 5 of Table I, application no. 15, has been published in B. And the activity of the gene product thereof is the activity of b1742-protein.

Accordingly, in one embodiment, the process of the present invention for producing succinate in a non-human organism, like a microorganism or a plant or a part thereof, comprises increasing or generating the activity of a gene product with the activity of a gene product conferring the activity of “b1742-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said B1742, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B1742, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B1742, or a functional equivalent or a homolog thereof as depicted in column 8 of Table II, application no. 15, preferably a homolog or functional equivalent as depicted in column 8 of Table II B, application no. 15, and being depicted in the same respective line as said B1742, and preferably the activity is increased non-targeted, whereby the respective line disclose in column 7 the fine chemical succinate.

Accordingly, in one embodiment, the molecule which activity is to be increased in the process of the invention is the gene product with an activity as a “b1742-protein”, preferably it is the molecule of section (a) or (b) of this paragraph.

In particular, it was observed that in plants, especially in Arabidopsis thaliana, increasing or generating the activity of a gene product non-targeted with the activity of a “b1742-protein”, preferably being encoded by a gene comprising the nucleic acid sequence SEQ ID NO. 134762, preferably the coding region thereof, conferred the production of or the increase in succinate compared with the wild type control.

The nucleic acid sequence of B1747 from Escherichia coli, e.g. as shown in the respective line in column 5 of Table I, application no. 15, has been published in B. And the activity of the gene product thereof is the activity of arginine N-succinyltransferase.

Accordingly, in one embodiment, the process of the present invention for producing succinate in a non-human organism, like a microorganism or a plant or a part thereof, comprises increasing or generating the activity of a gene product with the activity of a gene product conferring the activity of “arginine N-succinyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said B1747, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B1747, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B1747, or a functional equivalent or a homolog thereof as depicted in column 8 of Table II, application no. 15, preferably a homolog or functional equivalent as depicted in column 8 of Table II B, application no. 15, and being depicted in the same respective line as said B1747, and preferably the activity is increased plastidic, whereby the respective line disclose in column 7 the fine chemical succinate.

Accordingly, in one embodiment, the molecule which activity is to be increased in the process of the invention is the gene product with an activity as a “arginine N-succinyltransferase”, preferably it is the molecule of section (a) or (b) of this paragraph.

In particular, it was observed that in plants, especially in Arabidopsis thaliana, increasing or generating the activity of a gene product plastidic with the activity of a “arginine N-succinyltransferase”, preferably being encoded by a gene comprising the nucleic acid sequence SEQ ID NO. 78779, preferably the coding region thereof, conferred the production of or the increase in succinate compared with the wild type control.

The nucleic acid sequence of B1755 from Escherichia coli, e.g. as shown in the respective line in column 5 of Table I, application no. 15, has been published in B. And the activity of the gene product thereof is the activity of ABC transporter permease protein.

Accordingly, in one embodiment, the process of the present invention for producing vitamin c and/or threonic acid in a non-human organism, like a microorganism or a plant or a part thereof, comprises increasing or generating the activity of a gene product with the activity of a gene product conferring the activity of “ABC transporter permease protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical vitamin c and/or threonic acid), application no. 15, preferably the coding region thereof, or a homolog or a fragment thereof, and being depicted in the same respective line as said B1755, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B1755, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B1755, or a functional equivalent or a homolog thereof as depicted in column 8 of Table II, application no. 15, preferably a homolog or functional equivalent as depicted in column 8 of Table II B, application no. 15, and being depicted in the same respective line as said B1755, and preferably the activity is increased non-targeted, whereby the respective line disclose in column 7 the fine chemical vitamin c and/or threonic acid.

Accordingly, in one embodiment, the molecule which activity is to be increased in the process of the invention is the gene product with an activity as a “ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “ABC transporter permease protein”, preferably being encoded by a gene comprising the nucleic acid sequence SEQ ID NO. 78852, preferably the coding region thereof, conferred the production of or the increase in vitamin c and/or threonic acid compared with the wild type control.

The nucleic acid sequence of B1845 from Escherichia coli, e.g. as shown in the respective line in column 5 of Table I, application no. 15, has been published in B. And the activity of the gene product thereof is the activity of protease.

Accordingly, in one embodiment, the process of the present invention for producing malate in a non-human organism, like a microorganism or a plant or a part thereof, comprises increasing or generating the activity of a gene product with the activity of a gene product conferring the activity of “protease”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said B1845, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B1845, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B1845, or a functional equivalent or a homolog thereof as depicted in column 8 of Table II, application no. 15, preferably a homolog or functional equivalent as depicted in column 8 of Table II B, application no. 15, and being depicted in the same respective line as said B1845, and preferably the activity is increased non-targeted, whereby the respective line disclose in column 7 the fine chemical malate.

Accordingly, in one embodiment, the molecule which activity is to be increased in the process of the invention is the gene product with an activity as a “protease”, preferably it is the molecule of section (a) or (b) of this paragraph.

In particular, it was observed that in plants, especially in Arabidopsis thaliana, increasing or generating the activity of a gene product non-targeted with the activity of a “protease”, preferably being encoded by a gene comprising the nucleic acid sequence SEQ ID NO. 38573, preferably the coding region thereof, conferred the production of or the increase in malate compared with the wild type control.

The nucleic acid sequence of B1865 from Escherichia coli, e.g. as shown in the respective line in column 5 of Table I, application no. 15, has been published in B. And the activity of the gene product thereof is the activity of dATP pyrophosphohydrolase.

Accordingly, in one embodiment, the process of the present invention for producing vitamin c and/or threonic acid in a non-human organism, like a microorganism or a plant or a part thereof, comprises increasing or generating the activity of a gene product with the activity of a gene product conferring the activity of “dATP pyrophosphohydrolase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical vitamin c and/or threonic acid), application no. 15, preferably the coding region thereof, or a homolog or a fragment thereof, and being depicted in the same respective line as said B1865, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B1865, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B1865, or a functional equivalent or a homolog thereof as depicted in column 8 of Table II, application no. 15, preferably a homolog or functional equivalent as depicted in column 8 of Table II B, application no. 15, and being depicted in the same respective line as said B1865, and preferably the activity is increased non-targeted, whereby the respective line disclose in column 7 the fine chemical vitamin c and/or threonic acid.

Accordingly, in one embodiment, the molecule which activity is to be increased in the process of the invention is the gene product with an activity as a “dATP pyrophosphohydrolase”, preferably it is the molecule of section (a) or (b) of this paragraph.

In particular, it was observed that in plants, especially in Arabidopsis thaliana, increasing or generating the activity of a gene product non-targeted with the activity of a “dATP pyrophosphohydrolase”, preferably being encoded by a gene comprising the nucleic acid sequence SEQ ID NO. 134783, preferably the coding region thereof, conferred the production of or the increase in vitamin c and/or threonic acid compared with the wild type control.

The nucleic acid sequence of B1898 from Escherichia coli, e.g. as shown in the respective line in column 5 of Table I, application no. 15, has been published in B. And the activity of the gene product thereof is the activity of b1898-protein.

Accordingly, in one embodiment, the process of the present invention for producing succinate in a non-human organism, like a microorganism or a plant or a part thereof, comprises increasing or generating the activity of a gene product with the activity of a gene product conferring the activity of “b1898-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said B1898, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B1898, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B1898, or a functional equivalent or a homolog thereof as depicted in column 8 of Table II, application no. 15, preferably a homolog or functional equivalent as depicted in column 8 of Table II B, application no. 15, and being depicted in the same respective line as said B1898, and preferably the activity is increased non-targeted, whereby the respective line disclose in column 7 the fine chemical succinate.

Accordingly, in one embodiment, the molecule which activity is to be increased in the process of the invention is the gene product with an activity as a “b1898-protein”, preferably it is the molecule of section (a) or (b) of this paragraph.

In particular, it was observed that in plants, especially in Arabidopsis thaliana, increasing or generating the activity of a gene product non-targeted with the activity of a “b1898-protein”, preferably being encoded by a gene comprising the nucleic acid sequence SEQ ID NO. 38767, preferably the coding region thereof, conferred the production of or the increase in succinate compared with the wild type control.

The nucleic acid sequence of B1938 from Escherichia coli, e.g. as shown in the respective line in column 5 of Table I, application no. 15, has been published in B. And the activity of the gene product thereof is the activity of flagellar M-ring protein.

Accordingly, in one embodiment, the process of the present invention for producing fumarate in a non-human organism, like a microorganism or a plant or a part thereof, comprises increasing or generating the activity of a gene product with the activity of a gene product conferring the activity of “flagellar M-ring protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said B1938, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B1938, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B1938, or a functional equivalent or a homolog thereof as depicted in column 8 of Table II, application no. 15, preferably a homolog or functional equivalent as depicted in column 8 of Table II B, application no. 15, and being depicted in the same respective line as said B1938, and preferably the activity is increased non-targeted, whereby the respective line disclose in column 7 the fine chemical fumarate.

Accordingly, in one embodiment, the molecule which activity is to be increased in the process of the invention is the gene product with an activity as a “flagellar M-ring protein”, preferably it is the molecule of section (a) or (b) of this paragraph.

In particular, it was observed that in plants, especially in Arabidopsis thaliana, increasing or generating the activity of a gene product non-targeted with the activity of a “flagellar M-ring protein”, preferably being encoded by a gene comprising the nucleic acid sequence SEQ ID NO. 134864, preferably the coding region thereof, conferred the production of or the increase in fumarate compared with the wild type control.

The nucleic acid sequence of B1938 from Escherichia coli, e.g. as shown in the respective line in column 5 of Table I, application no. 15, has been published in B. And the activity of the gene product thereof is the activity of flagellar M-ring protein.

Accordingly, in one embodiment, the process of the present invention for producing malate in a non-human organism, like a microorganism or a plant or a part thereof, comprises increasing or generating the activity of a gene product with the activity of a gene product conferring the activity of “flagellar M-ring protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said B1938, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B1938, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B1938, or a functional equivalent or a homolog thereof as depicted in column 8 of Table II, application no. 15, preferably a homolog or functional equivalent as depicted in column 8 of Table II B, application no. 15, and being depicted in the same respective line as said B1938, and preferably the activity is increased non-targeted, whereby the respective line disclose in column 7 the fine chemical malate.

Accordingly, in one embodiment, the molecule which activity is to be increased in the process of the invention is the gene product with an activity as a “flagellar M-ring protein”, preferably it is the molecule of section (a) or (b) of this paragraph.

In particular, it was observed that in plants, especially in Arabidopsis thaliana, increasing or generating the activity of a gene product non-targeted with the activity of a “flagellar M-ring protein”, preferably being encoded by a gene comprising the nucleic acid sequence SEQ ID NO. 134864, preferably the coding region thereof, conferred the production of or the increase in malate compared with the wild type control.

The nucleic acid sequence of B1938 from Escherichia coli, e.g. as shown in the respective line in column 5 of Table I, application no. 15, has been published in B. And the activity of the gene product thereof is the activity of flagellar M-ring protein.

Accordingly, in one embodiment, the process of the present invention for producing succinate in a non-human organism, like a microorganism or a plant or a part thereof, comprises increasing or generating the activity of a gene product with the activity of a gene product conferring the activity of “flagellar M-ring protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said B1938, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B1938, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B1938, or a functional equivalent or a homolog thereof as depicted in column 8 of Table II, application no. 15, preferably a homolog or functional equivalent as depicted in column 8 of Table II B, application no. 15, and being depicted in the same respective line as said B1938, and preferably the activity is increased non-targeted, whereby the respective line disclose in column 7 the fine chemical succinate.

Accordingly, in one embodiment, the molecule which activity is to be increased in the process of the invention is the gene product with an activity as a “flagellar M-ring protein”, preferably it is the molecule of section (a) or (b) of this paragraph.

In particular, it was observed that in plants, especially in Arabidopsis thaliana, increasing or generating the activity of a gene product non-targeted with the activity of a “flagellar M-ring protein”, preferably being encoded by a gene comprising the nucleic acid sequence SEQ ID NO. 134864, preferably the coding region thereof, conferred the production of or the increase in succinate compared with the wild type control.

The nucleic acid sequence of B2027 from Escherichia coli, e.g. as shown in the respective line in column 5 of Table I, application no. 15, has been published in B. And the activity of the gene product thereof is the activity of O-antigen chain length determinant.

Accordingly, in one embodiment, the process of the present invention for producing malate in a non-human organism, like a microorganism or a plant or a part thereof, comprises increasing or generating the activity of a gene product with the activity of a gene product conferring the activity of “O-antigen chain length determinant”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said B2027, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B2027, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B2027, or a functional equivalent or a homolog thereof as depicted in column 8 of Table II, application no. 15, preferably a homolog or functional equivalent as depicted in column 8 of Table II B, application no. 15, and being depicted in the same respective line as said B2027, and preferably the activity is increased non-targeted, whereby the respective line disclose in column 7 the fine chemical malate.

Accordingly, in one embodiment, the molecule which activity is to be increased in the process of the invention is the gene product with an activity as a “O-antigen chain length determinant”, preferably it is the molecule of section (a) or (b) of this paragraph.

In particular, it was observed that in plants, especially in Arabidopsis thaliana, increasing or generating the activity of a gene product non-targeted with the activity of a “O-antigen chain length determinant”, preferably being encoded by a gene comprising the nucleic acid sequence SEQ ID NO. 79217, preferably the coding region thereof, conferred the production of or the increase in malate compared with the wild type control.

The nucleic acid sequence of B2399 from Escherichia coli, e.g. as shown in the respective line in column 5 of Table I, application no. 15, has been published in B. And the activity of the gene product thereof is the activity of b2399-protein.

Accordingly, in one embodiment, the process of the present invention for producing fumarate in a non-human organism, like a microorganism or a plant or a part thereof, comprises increasing or generating the activity of a gene product with the activity of a gene product conferring the activity of “b2399-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said B2399, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B2399, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B2399, or a functional equivalent or a homolog thereof as depicted in column 8 of Table II, application no. 15, preferably a homolog or functional equivalent as depicted in column 8 of Table II B, application no. 15, and being depicted in the same respective line as said B2399, and preferably the activity is increased non-targeted, whereby the respective line disclose in column 7 the fine chemical fumarate.

Accordingly, in one embodiment, the molecule which activity is to be increased in the process of the invention is the gene product with an activity as a “b2399-protein”, preferably it is the molecule of section (a) or (b) of this paragraph.

In particular, it was observed that in plants, especially in Arabidopsis thaliana, increasing or generating the activity of a gene product non-targeted with the activity of a “b2399-protein”, preferably being encoded by a gene comprising the nucleic acid sequence SEQ ID NO. 39237, preferably the coding region thereof, conferred the production of or the increase in fumarate compared with the wild type control.

The nucleic acid sequence of B2399 from Escherichia coli, e.g. as shown in the respective line in column 5 of Table I, application no. 15, has been published in B. And the activity of the gene product thereof is the activity of b2399-protein.

Accordingly, in one embodiment, the process of the present invention for producing succinate in a non-human organism, like a microorganism or a plant or a part thereof, comprises increasing or generating the activity of a gene product with the activity of a gene product conferring the activity of “b2399-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said B2399, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B2399, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B2399, or a functional equivalent or a homolog thereof as depicted in column 8 of Table II, application no. 15, preferably a homolog or functional equivalent as depicted in column 8 of Table II B, application no. 15, and being depicted in the same respective line as said B2399, and preferably the activity is increased non-targeted, whereby the respective line disclose in column 7 the fine chemical succinate.

Accordingly, in one embodiment, the molecule which activity is to be increased in the process of the invention is the gene product with an activity as a “b2399-protein”, preferably it is the molecule of section (a) or (b) of this paragraph.

In particular, it was observed that in plants, especially in Arabidopsis thaliana, increasing or generating the activity of a gene product non-targeted with the activity of a “b2399-protein”, preferably being encoded by a gene comprising the nucleic acid sequence SEQ ID NO. 39237, preferably the coding region thereof, conferred the production of or the increase in succinate compared with the wild type control.

The nucleic acid sequence of B2399 from Escherichia coli, e.g. as shown in the respective line in column 5 of Table I, application no. 15, has been published in B. And the activity of the gene product thereof is the activity of b2399-protein.

Accordingly, in one embodiment, the process of the present invention for producing vitamin c and/or threonic acid in a non-human organism, like a microorganism or a plant or a part thereof, comprises increasing or generating the activity of a gene product with the activity of a gene product conferring the activity of “b2399-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical vitamin c and/or threonic acid), application no. 15, preferably the coding region thereof, or a homolog or a fragment thereof, and being depicted in the same respective line as said B2399, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B2399, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B2399, or a functional equivalent or a homolog thereof as depicted in column 8 of Table II, application no. 15, preferably a homolog or functional equivalent as depicted in column 8 of Table II B, application no. 15, and being depicted in the same respective line as said B2399, and preferably the activity is increased non-targeted, whereby the respective line disclose in column 7 the fine chemical vitamin c and/or threonic acid.

Accordingly, in one embodiment, the molecule which activity is to be increased in the process of the invention is the gene product with an activity as a “b2399-protein”, preferably it is the molecule of section (a) or (b) of this paragraph.

In particular, it was observed that in plants, especially in Arabidopsis thaliana, increasing or generating the activity of a gene product non-targeted with the activity of a “b2399-protein”, preferably being encoded by a gene comprising the nucleic acid sequence SEQ ID NO. 39237, preferably the coding region thereof, conferred the production of or the increase in vitamin c and/or threonic acid compared with the wild type control.

The nucleic acid sequence of B2414 from Escherichia coli, e.g. as shown in the respective line in column 5 of Table I, application no. 15, has been published in B. And the activity of the gene product thereof is the activity of cysteine synthase A.

Accordingly, in one embodiment, the process of the present invention for producing vitamin c and/or threonic acid in a non-human organism, like a microorganism or a plant or a part thereof, comprises increasing or generating the activity of a gene product with the activity of a gene product conferring the activity of “cysteine synthase A”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical vitamin c and/or threonic acid), application no. 15, preferably the coding region thereof, or a homolog or a fragment thereof, and being depicted in the same respective line as said B2414, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B2414, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B2414, or a functional equivalent or a homolog thereof as depicted in column 8 of Table II, application no. 15, preferably a homolog or functional equivalent as depicted in column 8 of Table II B, application no. 15, and being depicted in the same respective line as said B2414, and preferably the activity is increased non-targeted, whereby the respective line disclose in column 7 the fine chemical vitamin c and/or threonic acid.

Accordingly, in one embodiment, the molecule which activity is to be increased in the process of the invention is the gene product with an activity as a “cysteine synthase A”, preferably it is the molecule of section (a) or (b) of this paragraph.

In particular, it was observed that in plants, especially in Arabidopsis thaliana, increasing or generating the activity of a gene product non-targeted with the activity of a “cysteine synthase A”, preferably being encoded by a gene comprising the nucleic acid sequence SEQ ID NO. 39300, preferably the coding region thereof, conferred the production of or the increase in vitamin c and/or threonic acid compared with the wild type control.

The nucleic acid sequence of B2421 from Escherichia coli, e.g. as shown in the respective line in column 5 of Table I, application no. 15, has been published in B. And the activity of the gene product thereof is the activity of cysteine synthase B.

Accordingly, in one embodiment, the process of the present invention for producing vitamin c and/or threonic acid in a non-human organism, like a microorganism or a plant or a part thereof, comprises increasing or generating the activity of a gene product with the activity of a gene product conferring the activity of “cysteine synthase B”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical vitamin c and/or threonic acid), application no. 15, preferably the coding region thereof, or a homolog or a fragment thereof, and being depicted in the same respective line as said B2421, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B2421, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B2421, or a functional equivalent or a homolog thereof as depicted in column 8 of Table II, application no. 15, preferably a homolog or functional equivalent as depicted in column 8 of Table II B, application no. 15, and being depicted in the same respective line as said B2421, and preferably the activity is increased plastidic, whereby the respective line disclose in column 7 the fine chemical vitamin c and/or threonic acid.

Accordingly, in one embodiment, the molecule which activity is to be increased in the process of the invention is the gene product with an activity as a “cysteine synthase B”, preferably it is the molecule of section (a) or (b) of this paragraph.

In particular, it was observed that in plants, especially in Arabidopsis thaliana, increasing or generating the activity of a gene product plastidic with the activity of a “cysteine synthase B”, preferably being encoded by a gene comprising the nucleic acid sequence SEQ ID NO. 134924, preferably the coding region thereof, conferred the production of or the increase in vitamin c and/or threonic acid compared with the wild type control.

The nucleic acid sequence of B2634 from Escherichia coli, e.g. as shown in the respective line in column 5 of Table I, application no. 15, has been published in B. And the activity of the gene product thereof is the activity of transcriptional regulator.

Accordingly, in one embodiment, the process of the present invention for producing fumarate in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said B2634, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B2634, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B2634, or a functional equivalent or a homolog thereof as depicted in column 8 of Table II, application no. 15, preferably a homolog or functional equivalent as depicted in column 8 of Table II B, application no. 15, and being depicted in the same respective line as said B2634, and preferably the activity is increased non-targeted, whereby the respective line disclose in column 7 the fine chemical fumarate.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 40726, preferably the coding region thereof, conferred the production of or the increase in fumarate compared with the wild type control.

The nucleic acid sequence of B2634 from Escherichia coli, e.g. as shown in the respective line in column 5 of Table I, application no. 15, has been published in B. And the activity of the gene product thereof is the activity of transcriptional regulator.

Accordingly, in one embodiment, the process of the present invention for producing malate in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said B2634, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B2634, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B2634, or a functional equivalent or a homolog thereof as depicted in column 8 of Table II, application no. 15, preferably a homolog or functional equivalent as depicted in column 8 of Table II B, application no. 15, and being depicted in the same respective line as said B2634, and preferably the activity is increased non-targeted, whereby the respective line disclose in column 7 the fine chemical malate.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 40726, preferably the coding region thereof, conferred the production of or the increase in malate compared with the wild type control.

The nucleic acid sequence of B2701 from Escherichia coli, e.g. as shown in the respective line in column 5 of Table I, application no. 15, has been published in B. And the activity of the gene product thereof is the activity of murein transglycosylase.

Accordingly, in one embodiment, the process of the present invention for producing succinate in a non-human organism, like a microorganism or a plant or a part thereof, comprises increasing or generating the activity of a gene product with the activity of a gene product conferring the activity of “murein transglycosylase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said B2701, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B2701, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B2701, or a functional equivalent or a homolog thereof as depicted in column 8 of Table II, application no. 15, preferably a homolog or functional equivalent as depicted in column 8 of Table II B, application no. 15, and being depicted in the same respective line as said B2701, and preferably the activity is increased non-targeted, whereby the respective line disclose in column 7 the fine chemical succinate.

Accordingly, in one embodiment, the molecule which activity is to be increased in the process of the invention is the gene product with an activity as a “murein transglycosylase”, preferably it is the molecule of section (a) or (b) of this paragraph.

In particular, it was observed that in plants, especially in Arabidopsis thaliana, increasing or generating the activity of a gene product non-targeted with the activity of a “murein transglycosylase”, preferably being encoded by a gene comprising the nucleic acid sequence SEQ ID NO. 80756, preferably the coding region thereof, conferred the production of or the increase in succinate compared with the wild type control.

The nucleic acid sequence of B2747 from Escherichia coli, e.g. as shown in the respective line in column 5 of Table I, application no. 15, has been published in B. And the activity of the gene product thereof is the activity of 2-C-methyl-D-erythritol 4-phosphate cytidylyltransferase.

Accordingly, in one embodiment, the process of the present invention for producing succinate in a non-human organism, like a microorganism or a plant or a part thereof, comprises increasing or generating the activity of a gene product with the activity of a gene product conferring the activity of “2-C-methyl-D-erythritol 4-phosphate cytidylyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said B2747, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B2747, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B2747, or a functional equivalent or a homolog thereof as depicted in column 8 of Table II, application no. 15, preferably a homolog or functional equivalent as depicted in column 8 of Table II B, application no. 15, and being depicted in the same respective line as said B2747, and preferably the activity is increased non-targeted, whereby the respective line disclose in column 7 the fine chemical succinate.

Accordingly, in one embodiment, the molecule which activity is to be increased in the process of the invention is the gene product with an activity as a “2-C-methyl-D-erythritol 4-phosphate cytidylyltransferase”, preferably it is the molecule of section (a) or (b) of this paragraph.

In particular, it was observed that in plants, especially in Arabidopsis thaliana, increasing or generating the activity of a gene product non-targeted with the activity of a “2-C-methyl-D-erythritol 4-phosphate cytidylyltransferase”, preferably being encoded by a gene comprising the nucleic acid sequence SEQ ID NO. 81075, preferably the coding region thereof, conferred the production of or the increase in succinate compared with the wild type control.

The nucleic acid sequence of B2818 from Escherichia coli, e.g. as shown in the respective line in column 5 of Table I, application no. 15, has been published in B. And the activity of the gene product thereof is the activity of amino-acid acetyltransferase.

Accordingly, in one embodiment, the process of the present invention for producing succinate in a non-human organism, like a microorganism or a plant or a part thereof, comprises increasing or generating the activity of a gene product with the activity of a gene product conferring the activity of “amino-acid acetyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said B2818, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B2818, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B2818, or a functional equivalent or a homolog thereof as depicted in column 8 of Table II, application no. 15, preferably a homolog or functional equivalent as depicted in column 8 of Table II B, application no. 15, and being depicted in the same respective line as said B2818, and preferably the activity is increased non-targeted, whereby the respective line disclose in column 7 the fine chemical succinate.

Accordingly, in one embodiment, the molecule which activity is to be increased in the process of the invention is the gene product with an activity as a “amino-acid acetyltransferase”, preferably it is the molecule of section (a) or (b) of this paragraph.

In particular, it was observed that in plants, especially in Arabidopsis thaliana, increasing or generating the activity of a gene product non-targeted with the activity of a “amino-acid acetyltransferase”, preferably being encoded by a gene comprising the nucleic acid sequence SEQ ID NO. 81576, preferably the coding region thereof, conferred the production of or the increase in succinate compared with the wild type control.

The nucleic acid sequence of B2866 from Escherichia coli, e.g. as shown in the respective line in column 5 of Table I, application no. 15, has been published in B. And the activity of the gene product thereof is the activity of xanthine dehydrogenase.

Accordingly, in one embodiment, the process of the present invention for producing succinate in a non-human organism, like a microorganism or a plant or a part thereof, comprises increasing or generating the activity of a gene product with the activity of a gene product conferring the activity of “xanthine dehydrogenase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said B2866, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B2866, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B2866, or a functional equivalent or a homolog thereof as depicted in column 8 of Table II, application no. 15, preferably a homolog or functional equivalent as depicted in column 8 of Table II B, application no. 15, and being depicted in the same respective line as said B2866, and preferably the activity is increased non-targeted, whereby the respective line disclose in column 7 the fine chemical succinate.

Accordingly, in one embodiment, the molecule which activity is to be increased in the process of the invention is the gene product with an activity as a “xanthine dehydrogenase”, preferably it is the molecule of section (a) or (b) of this paragraph.

In particular, it was observed that in plants, especially in Arabidopsis thaliana, increasing or generating the activity of a gene product non-targeted with the activity of a “xanthine dehydrogenase”, preferably being encoded by a gene comprising the nucleic acid sequence SEQ ID NO. 115864, preferably the coding region thereof, conferred the production of or the increase in succinate compared with the wild type control.

The nucleic acid sequence of B3008 from Escherichia coli, e.g. as shown in the respective line in column 5 of Table I, application no. 15, has been published in B. And the activity of the gene product thereof is the activity of cystathionine beta-lyase.

Accordingly, in one embodiment, the process of the present invention for producing succinate in a non-human organism, like a microorganism or a plant or a part thereof, comprises increasing or generating the activity of a gene product with the activity of a gene product conferring the activity of “cystathionine beta-lyase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said B3008, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B3008, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B3008, or a functional equivalent or a homolog thereof as depicted in column 8 of Table II, application no. 15, preferably a homolog or functional equivalent as depicted in column 8 of Table II B, application no. 15, and being depicted in the same respective line as said B3008, and preferably the activity is increased non-targeted, whereby the respective line disclose in column 7 the fine chemical succinate.

Accordingly, in one embodiment, the molecule which activity is to be increased in the process of the invention is the gene product with an activity as a “cystathionine beta-lyase”, preferably it is the molecule of section (a) or (b) of this paragraph.

In particular, it was observed that in plants, especially in Arabidopsis thaliana, increasing or generating the activity of a gene product non-targeted with the activity of a “cystathionine betalyase”, preferably being encoded by a gene comprising the nucleic acid sequence SEQ ID NO. 135893, preferably the coding region thereof, conferred the production of or the increase in succinate compared with the wild type control.

The nucleic acid sequence of B3262 from Escherichia coli, e.g. as shown in the respective line in column 5 of Table I, application no. 15, has been published in B. And the activity of the gene product thereof is the activity of methyltransferase.

Accordingly, in one embodiment, the process of the present invention for producing fumarate in a non-human organism, like a microorganism or a plant or a part thereof, comprises increasing or generating the activity of a gene product with the activity of a gene product conferring the activity of “methyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said B3262, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B3262, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B3262, or a functional equivalent or a homolog thereof as depicted in column 8 of Table II, application no. 15, preferably a homolog or functional equivalent as depicted in column 8 of Table II B, application no. 15, and being depicted in the same respective line as said B3262, and preferably the activity is increased non-targeted, whereby the respective line disclose in column 7 the fine chemical fumarate.

Accordingly, in one embodiment, the molecule which activity is to be increased in the process of the invention is the gene product with an activity as a “methyltransferase”, preferably it is the molecule of section (a) or (b) of this paragraph.

In particular, it was observed that in plants, especially in Arabidopsis thaliana, increasing or generating the activity of a gene product non-targeted with the activity of a “methyltransferase”, preferably being encoded by a gene comprising the nucleic acid sequence SEQ ID NO. 42502, preferably the coding region thereof, conferred the production of or the increase in fumarate compared with the wild type control.

The nucleic acid sequence of B3262 from Escherichia coli, e.g. as shown in the respective line in column 5 of Table I, application no. 15, has been published in B. And the activity of the gene product thereof is the activity of methyltransferase.

Accordingly, in one embodiment, the process of the present invention for producing malate in a non-human organism, like a microorganism or a plant or a part thereof, comprises increasing or generating the activity of a gene product with the activity of a gene product conferring the activity of “methyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said B3262, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B3262, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B3262, or a functional equivalent or a homolog thereof as depicted in column 8 of Table II, application no. 15, preferably a homolog or functional equivalent as depicted in column 8 of Table II B, application no. 15, and being depicted in the same respective line as said B3262, and preferably the activity is increased non-targeted, whereby the respective line disclose in column 7 the fine chemical malate.

Accordingly, in one embodiment, the molecule which activity is to be increased in the process of the invention is the gene product with an activity as a “methyltransferase”, preferably it is the molecule of section (a) or (b) of this paragraph.

In particular, it was observed that in plants, especially in Arabidopsis thaliana, increasing or generating the activity of a gene product non-targeted with the activity of a “methyltransferase”, preferably being encoded by a gene comprising the nucleic acid sequence SEQ ID NO. 42502, preferably the coding region thereof, conferred the production of or the increase in malate compared with the wild type control.

The nucleic acid sequence of B3624 from Escherichia coli, e.g. as shown in the respective line in column 5 of Table I, application no. 15, has been published in B. And the activity of the gene product thereof is the activity of lipopolysaccharide core biosynthesis protein.

Accordingly, in one embodiment, the process of the present invention for producing fumarate in a non-human organism, like a microorganism or a plant or a part thereof, comprises increasing or generating the activity of a gene product with the activity of a gene product conferring the activity of “lipopolysaccharide core biosynthesis protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said B3624, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B3624, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B3624, or a functional equivalent or a homolog thereof as depicted in column 8 of Table II, application no. 15, preferably a homolog or functional equivalent as depicted in column 8 of Table II B, application no. 15, and being depicted in the same respective line as said B3624, and preferably the activity is increased non-targeted, whereby the respective line disclose in column 7 the fine chemical fumarate.

Accordingly, in one embodiment, the molecule which activity is to be increased in the process of the invention is the gene product with an activity as a “lipopolysaccharide core biosynthesis protein”, preferably it is the molecule of section (a) or (b) of this paragraph.

In particular, it was observed that in plants, especially in Arabidopsis thaliana, increasing or generating the activity of a gene product non-targeted with the activity of a “lipopolysaccharide core biosynthesis protein”, preferably being encoded by a gene comprising the nucleic acid sequence SEQ ID NO. 136077, preferably the coding region thereof, conferred the production of or the increase in fumarate compared with the wild type control.

The nucleic acid sequence of B3777 from Escherichia coli, e.g. as shown in the respective line in column 5 of Table I, application no. 15, has been published in B. And the activity of the gene product thereof is the activity of b3777-protein.

Accordingly, in one embodiment, the process of the present invention for producing succinate in a non-human organism, like a microorganism or a plant or a part thereof, comprises increasing or generating the activity of a gene product with the activity of a gene product conferring the activity of “b3777-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said B3777, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B3777, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B3777, or a functional equivalent or a homolog thereof as depicted in column 8 of Table II, application no. 15, preferably a homolog or functional equivalent as depicted in column 8 of Table II B, application no. 15, and being depicted in the same respective line as said B3777, and preferably the activity is increased non-targeted, whereby the respective line disclose in column 7 the fine chemical succinate.

Accordingly, in one embodiment, the molecule which activity is to be increased in the process of the invention is the gene product with an activity as a “b3777-protein”, preferably it is the molecule of section (a) or (b) of this paragraph.

In particular, it was observed that in plants, especially in Arabidopsis thaliana, increasing or generating the activity of a gene product non-targeted with the activity of a “b3777-protein”, preferably being encoded by a gene comprising the nucleic acid sequence SEQ ID NO. 136098, preferably the coding region thereof, conferred the production of or the increase in succinate compared with the wild type control.

The nucleic acid sequence of B3791 from Escherichia coli, e.g. as shown in the respective line in column 5 of Table I, application no. 15, has been published in B. And the activity of the gene product thereof is the activity of b3791-protein.

Accordingly, in one embodiment, the process of the present invention for producing malate in a non-human organism, like a microorganism or a plant or a part thereof, comprises increasing or generating the activity of a gene product with the activity of a gene product conferring the activity of “b3791-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said B3791, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B3791, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B3791, or a functional equivalent or a homolog thereof as depicted in column 8 of Table II, application no. 15, preferably a homolog or functional equivalent as depicted in column 8 of Table II B, application no. 15, and being depicted in the same respective line as said B3791, and preferably the activity is increased non-targeted, whereby the respective line disclose in column 7 the fine chemical malate.

Accordingly, in one embodiment, the molecule which activity is to be increased in the process of the invention is the gene product with an activity as a “b3791-protein”, preferably it is the molecule of section (a) or (b) of this paragraph.

In particular, it was observed that in plants, especially in Arabidopsis thaliana, increasing or generating the activity of a gene product non-targeted with the activity of a “b3791-protein”, preferably being encoded by a gene comprising the nucleic acid sequence SEQ ID NO. 83726, preferably the coding region thereof, conferred the production of or the increase in malate compared with the wild type control.

The nucleic acid sequence of B3791 from Escherichia coli, e.g. as shown in the respective line in column 5 of Table I, application no. 15, has been published in B. And the activity of the gene product thereof is the activity of b3791-protein.

Accordingly, in one embodiment, the process of the present invention for producing succinate in a non-human organism, like a microorganism or a plant or a part thereof, comprises increasing or generating the activity of a gene product with the activity of a gene product conferring the activity of “b3791-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said B3791, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B3791, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B3791, or a functional equivalent or a homolog thereof as depicted in column 8 of Table II, application no. 15, preferably a homolog or functional equivalent as depicted in column 8 of Table II B, application no. 15, and being depicted in the same respective line as said B3791, and preferably the activity is increased non-targeted, whereby the respective line disclose in column 7 the fine chemical succinate.

Accordingly, in one embodiment, the molecule which activity is to be increased in the process of the invention is the gene product with an activity as a “b3791-protein”, preferably it is the molecule of section (a) or (b) of this paragraph.

In particular, it was observed that in plants, especially in Arabidopsis thaliana, increasing or generating the activity of a gene product non-targeted with the activity of a “b3791-protein”, preferably being encoded by a gene comprising the nucleic acid sequence SEQ ID NO. 83726, preferably the coding region thereof, conferred the production of or the increase in succinate compared with the wild type control.

The nucleic acid sequence of B3814 from Escherichia coli, e.g. as shown in the respective line in column 5 of Table I, application no. 15, has been published in B. And the activity of the gene product thereof is the activity of b3814-protein.

Accordingly, in one embodiment, the process of the present invention for producing citramalate in a non-human organism, like a microorganism or a plant or a part thereof, comprises increasing or generating the activity of a gene product with the activity of a gene product conferring the activity of “b3814-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said B3814, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B3814, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B3814, or a functional equivalent or a homolog thereof as depicted in column 8 of Table II, application no. 15, preferably a homolog or functional equivalent as depicted in column 8 of Table II B, application no. 15, and being depicted in the same respective line as said B3814, and preferably the activity is increased non-targeted, whereby the respective line disclose in column 7 the fine chemical citramalate.

Accordingly, in one embodiment, the molecule which activity is to be increased in the process of the invention is the gene product with an activity as a “b3814-protein”, preferably it is the molecule of section (a) or (b) of this paragraph.

In particular, it was observed that in plants, especially in Arabidopsis thaliana, increasing or generating the activity of a gene product non-targeted with the activity of a “b3814-protein”, preferably being encoded by a gene comprising the nucleic acid sequence SEQ ID NO. 44196, preferably the coding region thereof, conferred the production of or the increase in citramalate compared with the wild type control.

The nucleic acid sequence of B3817 from Escherichia coli, e.g. as shown in the respective line in column 5 of Table I, application no. 15, has been published in B. And the activity of the gene product thereof is the activity of b3817-protein.

Accordingly, in one embodiment, the process of the present invention for producing fumarate in a non-human organism, like a microorganism or a plant or a part thereof, comprises increasing or generating the activity of a gene product with the activity of a gene product conferring the activity of “b3817-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said B3817, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B3817, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B3817, or a functional equivalent or a homolog thereof as depicted in column 8 of Table II, application no. 15, preferably a homolog or functional equivalent as depicted in column 8 of Table II B, application no. 15, and being depicted in the same respective line as said B3817, and preferably the activity is increased non-targeted, whereby the respective line disclose in column 7 the fine chemical fumarate.

Accordingly, in one embodiment, the molecule which activity is to be increased in the process of the invention is the gene product with an activity as a “b3817-protein”, preferably it is the molecule of section (a) or (b) of this paragraph.

In particular, it was observed that in plants, especially in Arabidopsis thaliana, increasing or generating the activity of a gene product non-targeted with the activity of a “b3817-protein”, preferably being encoded by a gene comprising the nucleic acid sequence SEQ ID NO. 10708, preferably the coding region thereof, conferred the production of or the increase in fumarate compared with the wild type control.

The nucleic acid sequence of B3823 from Escherichia coli, e.g. as shown in the respective line in column 5 of Table I, application no. 15, has been published in B. And the activity of the gene product thereof is the activity of threonine efflux protein.

Accordingly, in one embodiment, the process of the present invention for producing citramalate in a non-human organism, like a microorganism or a plant or a part thereof, comprises increasing or generating the activity of a gene product with the activity of a gene product conferring the activity of “threonine efflux protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said B3823, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B3823, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B3823, or a functional equivalent or a homolog thereof as depicted in column 8 of Table II, application no. 15, preferably a homolog or functional equivalent as depicted in column 8 of Table II B, application no. 15, and being depicted in the same respective line as said B3823, and preferably the activity is increased non-targeted, whereby the respective line disclose in column 7 the fine chemical citramalate.

Accordingly, in one embodiment, the molecule which activity is to be increased in the process of the invention is the gene product with an activity as a “threonine efflux protein”, preferably it is the molecule of section (a) or (b) of this paragraph.

In particular, it was observed that in plants, especially in Arabidopsis thaliana, increasing or generating the activity of a gene product non-targeted with the activity of a “threonine efflux protein”, preferably being encoded by a gene comprising the nucleic acid sequence SEQ ID NO. 10726, preferably the coding region thereof, conferred the production of or the increase in citramalate compared with the wild type control.

The nucleic acid sequence of B3823 from Escherichia coli, e.g. as shown in the respective line in column 5 of Table I, application no. 15, has been published in B. And the activity of the gene product thereof is the activity of threonine efflux protein.

Accordingly, in one embodiment, the process of the present invention for producing malate in a non-human organism, like a microorganism or a plant or a part thereof, comprises increasing or generating the activity of a gene product with the activity of a gene product conferring the activity of “threonine efflux protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said B3823, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B3823, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B3823, or a functional equivalent or a homolog thereof as depicted in column 8 of Table II, application no. 15, preferably a homolog or functional equivalent as depicted in column 8 of Table II B, application no. 15, and being depicted in the same respective line as said B3823, and preferably the activity is increased non-targeted, whereby the respective line disclose in column 7 the fine chemical malate.

Accordingly, in one embodiment, the molecule which activity is to be increased in the process of the invention is the gene product with an activity as a “threonine efflux protein”, preferably it is the molecule of section (a) or (b) of this paragraph.

In particular, it was observed that in plants, especially in Arabidopsis thaliana, increasing or generating the activity of a gene product non-targeted with the activity of a “threonine efflux protein”, preferably being encoded by a gene comprising the nucleic acid sequence SEQ ID NO. 10726, preferably the coding region thereof, conferred the production of or the increase in malate compared with the wild type control.

The nucleic acid sequence of B3989 from Escherichia coli, e.g. as shown in the respective line in column 5 of Table I, application no. 15, has been published in B. And the activity of the gene product thereof is the activity of b3989-protein.

Accordingly, in one embodiment, the process of the present invention for producing succinate in a non-human organism, like a microorganism or a plant or a part thereof, comprises increasing or generating the activity of a gene product with the activity of a gene product conferring the activity of “b3989-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said B3989, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B3989, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B3989, or a functional equivalent or a homolog thereof as depicted in column 8 of Table II, application no. 15, preferably a homolog or functional equivalent as depicted in column 8 of Table II B, application no. 15, and being depicted in the same respective line as said B3989, and preferably the activity is increased non-targeted, whereby the respective line disclose in column 7 the fine chemical succinate.

Accordingly, in one embodiment, the molecule which activity is to be increased in the process of the invention is the gene product with an activity as a “b3989-protein”, preferably it is the molecule of section (a) or (b) of this paragraph.

In particular, it was observed that in plants, especially in Arabidopsis thaliana, increasing or generating the activity of a gene product non-targeted with the activity of a “b3989-protein”, preferably being encoded by a gene comprising the nucleic acid sequence SEQ ID NO. 44372, preferably the coding region thereof, conferred the production of or the increase in succinate compared with the wild type control.

The nucleic acid sequence of B4012 from Escherichia coli, e.g. as shown in the respective line in column 5 of Table I, application no. 15, has been published in B. And the activity of the gene product thereof is the activity of acetyltransferase.

Accordingly, in one embodiment, the process of the present invention for producing fumarate in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said B4012, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B4012, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B4012, or a functional equivalent or a homolog thereof as depicted in column 8 of Table II, application no. 15, preferably a homolog or functional equivalent as depicted in column 8 of Table II B, application no. 15, and being depicted in the same respective line as said B4012, and preferably the activity is increased non-targeted, whereby the respective line disclose in column 7 the fine chemical fumarate.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 44378, preferably the coding region thereof, conferred the production of or the increase in fumarate compared with the wild type control.

The nucleic acid sequence of B4029 from Escherichia coli, e.g. as shown in the respective line in column 5 of Table I, application no. 15, has been published in B. And the activity of the gene product thereof is the activity of b4029-protein.

Accordingly, in one embodiment, the process of the present invention for producing fumarate in a non-human organism, like a microorganism or a plant or a part thereof, comprises increasing or generating the activity of a gene product with the activity of a gene product conferring the activity of “b4029-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said B4029, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B4029, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B4029, or a functional equivalent or a homolog thereof as depicted in column 8 of Table II, application no. 15, preferably a homolog or functional equivalent as depicted in column 8 of Table II B, application no. 15, and being depicted in the same respective line as said B4029, and preferably the activity is increased non-targeted, whereby the respective line disclose in column 7 the fine chemical fumarate.

Accordingly, in one embodiment, the molecule which activity is to be increased in the process of the invention is the gene product with an activity as a “b4029-protein”, preferably it is the molecule of section (a) or (b) of this paragraph.

In particular, it was observed that in plants, especially in Arabidopsis thaliana, increasing or generating the activity of a gene product non-targeted with the activity of a “b4029-protein”, preferably being encoded by a gene comprising the nucleic acid sequence SEQ ID NO. 10740, preferably the coding region thereof, conferred the production of or the increase in fumarate compared with the wild type control.

The nucleic acid sequence of B4029 from Escherichia coli, e.g. as shown in the respective line in column 5 of Table I, application no. 15, has been published in B. And the activity of the gene product thereof is the activity of b4029-protein.

Accordingly, in one embodiment, the process of the present invention for producing malate in a non-human organism, like a microorganism or a plant or a part thereof, comprises increasing or generating the activity of a gene product with the activity of a gene product conferring the activity of “b4029-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said B4029, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B4029, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B4029, or a functional equivalent or a homolog thereof as depicted in column 8 of Table II, application no. 15, preferably a homolog or functional equivalent as depicted in column 8 of Table II B, application no. 15, and being depicted in the same respective line as said B4029, and preferably the activity is increased non-targeted, whereby the respective line disclose in column 7 the fine chemical malate.

Accordingly, in one embodiment, the molecule which activity is to be increased in the process of the invention is the gene product with an activity as a “b4029-protein”, preferably it is the molecule of section (a) or (b) of this paragraph.

In particular, it was observed that in plants, especially in Arabidopsis thaliana, increasing or generating the activity of a gene product non-targeted with the activity of a “b4029-protein”, preferably being encoded by a gene comprising the nucleic acid sequence SEQ ID NO. 10740, preferably the coding region thereof, conferred the production of or the increase in malate compared with the wild type control.

The nucleic acid sequence of B4029 from Escherichia coli, e.g. as shown in the respective line in column 5 of Table I, application no. 15, has been published in B. And the activity of the gene product thereof is the activity of b4029-protein.

Accordingly, in one embodiment, the process of the present invention for producing succinate in a non-human organism, like a microorganism or a plant or a part thereof, comprises increasing or generating the activity of a gene product with the activity of a gene product conferring the activity of “b4029-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said B4029, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B4029, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B4029, or a functional equivalent or a homolog thereof as depicted in column 8 of Table II, application no. 15, preferably a homolog or functional equivalent as depicted in column 8 of Table II B, application no. 15, and being depicted in the same respective line as said B4029, and preferably the activity is increased non-targeted, whereby the respective line disclose in column 7 the fine chemical succinate.

Accordingly, in one embodiment, the molecule which activity is to be increased in the process of the invention is the gene product with an activity as a “b4029-protein”, preferably it is the molecule of section (a) or (b) of this paragraph.

In particular, it was observed that in plants, especially in Arabidopsis thaliana, increasing or generating the activity of a gene product non-targeted with the activity of a “b4029-protein”, preferably being encoded by a gene comprising the nucleic acid sequence SEQ ID NO. 10740, preferably the coding region thereof, conferred the production of or the increase in succinate compared with the wild type control.

The nucleic acid sequence of B4050 from Escherichia coli, e.g. as shown in the respective line in column 5 of Table I, application no. 15, has been published in B. And the activity of the gene product thereof is the activity of b4050-protein.

Accordingly, in one embodiment, the process of the present invention for producing fumarate in a non-human organism, like a microorganism or a plant or a part thereof, comprises increasing or generating the activity of a gene product with the activity of a gene product conferring the activity of “b4050-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said B4050, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B4050, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B4050, or a functional equivalent or a homolog thereof as depicted in column 8 of Table II, application no. 15, preferably a homolog or functional equivalent as depicted in column 8 of Table II B, application no. 15, and being depicted in the same respective line as said B4050, and preferably the activity is increased non-targeted, whereby the respective line disclose in column 7 the fine chemical fumarate.

Accordingly, in one embodiment, the molecule which activity is to be increased in the process of the invention is the gene product with an activity as a “b4050-protein”, preferably it is the molecule of section (a) or (b) of this paragraph.

In particular, it was observed that in plants, especially in Arabidopsis thaliana, increasing or generating the activity of a gene product non-targeted with the activity of a “b4050-protein”, preferably being encoded by a gene comprising the nucleic acid sequence SEQ ID NO. 106532, preferably the coding region thereof, conferred the production of or the increase in fumarate compared with the wild type control.

The nucleic acid sequence of B4050 from Escherichia coli, e.g. as shown in the respective line in column 5 of Table I, application no. 15, has been published in B. And the activity of the gene product thereof is the activity of b4050-protein.

Accordingly, in one embodiment, the process of the present invention for producing malate in a non-human organism, like a microorganism or a plant or a part thereof, comprises increasing or generating the activity of a gene product with the activity of a gene product conferring the activity of “b4050-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said B4050, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B4050, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B4050, or a functional equivalent or a homolog thereof as depicted in column 8 of Table II, application no. 15, preferably a homolog or functional equivalent as depicted in column 8 of Table II B, application no. 15, and being depicted in the same respective line as said B4050, and preferably the activity is increased non-targeted, whereby the respective line disclose in column 7 the fine chemical malate.

Accordingly, in one embodiment, the molecule which activity is to be increased in the process of the invention is the gene product with an activity as a “b4050-protein”, preferably it is the molecule of section (a) or (b) of this paragraph.

In particular, it was observed that in plants, especially in Arabidopsis thaliana, increasing or generating the activity of a gene product non-targeted with the activity of a “b4050-protein”, preferably being encoded by a gene comprising the nucleic acid sequence SEQ ID NO. 106532, preferably the coding region thereof, conferred the production of or the increase in malate compared with the wild type control.

The nucleic acid sequence of B4056 from Escherichia coli, e.g. as shown in the respective line in column 5 of Table I, application no. 15, has been published in B. And the activity of the gene product thereof is the activity of b4056-protein.

Accordingly, in one embodiment, the process of the present invention for producing citramalate in a non-human organism, like a microorganism or a plant or a part thereof, comprises increasing or generating the activity of a gene product with the activity of a gene product conferring the activity of “b4056-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said B4056, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B4056, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B4056, or a functional equivalent or a homolog thereof as depicted in column 8 of Table II, application no. 15, preferably a homolog or functional equivalent as depicted in column 8 of Table II B, application no. 15, and being depicted in the same respective line as said B4056, and preferably the activity is increased non-targeted, whereby the respective line disclose in column 7 the fine chemical citramalate.

Accordingly, in one embodiment, the molecule which activity is to be increased in the process of the invention is the gene product with an activity as a “b4056-protein”, preferably it is the molecule of section (a) or (b) of this paragraph.

In particular, it was observed that in plants, especially in Arabidopsis thaliana, increasing or generating the activity of a gene product non-targeted with the activity of a “b4056-protein”, preferably being encoded by a gene comprising the nucleic acid sequence SEQ ID NO. 94542, preferably the coding region thereof, conferred the production of or the increase in citramalate compared with the wild type control.

The nucleic acid sequence of B4139 from Escherichia coli, e.g. as shown in the respective line in column 5 of Table I, application no. 15, has been published in B. And the activity of the gene product thereof is the activity of aspartase.

Accordingly, in one embodiment, the process of the present invention for producing citrate in a non-human organism, like a microorganism or a plant or a part thereof, comprises increasing or generating the activity of a gene product with the activity of a gene product conferring the activity of “aspartase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical citrate), application no. 15, preferably the coding region thereof, or a homolog or a fragment thereof, and being depicted in the same respective line as said B4139, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B4139, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B4139, or a functional equivalent or a homolog thereof as depicted in column 8 of Table II, application no. 15, preferably a homolog or functional equivalent as depicted in column 8 of Table II B, application no. 15, and being depicted in the same respective line as said B4139, and preferably the activity is increased non-targeted, whereby the respective line disclose in column 7 the fine chemical citrate.

Accordingly, in one embodiment, the molecule which activity is to be increased in the process of the invention is the gene product with an activity as a “aspartase”, preferably it is the molecule of section (a) or (b) of this paragraph.

In particular, it was observed that in plants, especially in Arabidopsis thaliana, increasing or generating the activity of a gene product non-targeted with the activity of a “aspartase”, preferably being encoded by a gene comprising the nucleic acid sequence SEQ ID NO. 45022, preferably the coding region thereof, conferred the production of or the increase in citrate compared with the wild type control.

The nucleic acid sequence of B4256 from Escherichia coli, e.g. as shown in the respective line in column 5 of Table I, application no. 15, has been published in B. And the activity of the gene product thereof is the activity of acetyltransferase.

Accordingly, in one embodiment, the process of the present invention for producing fumarate in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said B4256, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B4256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B4256, or a functional equivalent or a homolog thereof as depicted in column 8 of Table II, application no. 15, preferably a homolog or functional equivalent as depicted in column 8 of Table II B, application no. 15, and being depicted in the same respective line as said B4256, and preferably the activity is increased non-targeted, whereby the respective line disclose in column 7 the fine chemical fumarate.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 45321, preferably the coding region thereof, conferred the production of or the increase in fumarate compared with the wild type control.

The nucleic acid sequence of B4256 from Escherichia coli, e.g. as shown in the respective line in column 5 of Table I, application no. 15, has been published in B. And the activity of the gene product thereof is the activity of acetyltransferase.

Accordingly, in one embodiment, the process of the present invention for producing malate in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said B4256, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B4256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B4256, or a functional equivalent or a homolog thereof as depicted in column 8 of Table II, application no. 15, preferably a homolog or functional equivalent as depicted in column 8 of Table II B, application no. 15, and being depicted in the same respective line as said B4256, and preferably the activity is increased non-targeted, whereby the respective line disclose in column 7 the fine chemical malate.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 45321, preferably the coding region thereof, conferred the production of or the increase in malate compared with the wild type control.

The nucleic acid sequence of B4384 from Escherichia coli, e.g. as shown in the respective line in column 5 of Table I, application no. 15, has been published in B. And the activity of the gene product thereof is the activity of purine-nucleoside phosphorylase.

Accordingly, in one embodiment, the process of the present invention for producing vitamin c and/or threonic acid in a non-human organism, like a microorganism or a plant or a part thereof, comprises increasing or generating the activity of a gene product with the activity of a gene product conferring the activity of “purine-nucleoside phosphorylase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical vitamin c and/or threonic acid), application no. 15, preferably the coding region thereof, or a homolog or a fragment thereof, and being depicted in the same respective line as said B4384, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said B4384, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said B4384, or a functional equivalent or a homolog thereof as depicted in column 8 of Table II, application no. 15, preferably a homolog or functional equivalent as depicted in column 8 of Table II B, application no. 15, and being depicted in the same respective line as said B4384, and preferably the activity is increased non-targeted, whereby the respective line disclose in column 7 the fine chemical vitamin c and/or threonic acid.

Accordingly, in one embodiment, the molecule which activity is to be increased in the process of the invention is the gene product with an activity as a “purine-nucleoside phosphorylase”, preferably it is the molecule of section (a) or (b) of this paragraph.

In particular, it was observed that in plants, especially in Arabidopsis thaliana, increasing or generating the activity of a gene product non-targeted with the activity of a “purine-nucleoside phosphorylase”, preferably being encoded by a gene comprising the nucleic acid sequence SEQ ID NO. 45556, preferably the coding region thereof, conferred the production of or the increase in vitamin c and/or threonic acid compared with the wild type control.

The nucleic acid sequence of D90900 from Synechocystis sp., e.g. as shown in the respective line in column 5 of Table I, application no. 15, has been published in C. And the activity of the gene product thereof is the activity of lysine decarboxylase.

Accordingly, in one embodiment, the process of the present invention for producing succinate in a non-human organism, like a microorganism or a plant or a part thereof, comprises increasing or generating the activity of a gene product with the activity of a gene product conferring the activity of “lysine decarboxylase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said D90900, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said D90900, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said D90900, or a functional equivalent or a homolog thereof as depicted in column 8 of Table II, application no. 15, preferably a homolog or functional equivalent as depicted in column 8 of Table II B, application no. 15, and being depicted in the same respective line as said D90900, and preferably the activity is increased plastidic, whereby the respective line disclose in column 7 the fine chemical succinate.

Accordingly, in one embodiment, the molecule which activity is to be increased in the process of the invention is the gene product with an activity as a “lysine decarboxylase”, preferably it is the molecule of section (a) or (b) of this paragraph.

In particular, it was observed that in plants, especially in Arabidopsis thaliana, increasing or generating the activity of a gene product plastidic with the activity of a “lysine decarboxylase”, preferably being encoded by a gene comprising the nucleic acid sequence SEQ ID NO. 84079, preferably the coding region thereof, conferred the production of or the increase in succinate compared with the wild type control.

The nucleic acid sequence of GM02LC12622 from Glycine max, e.g. as shown in the respective line in column 5 of Table I, application no. 15, is unpublished. And the activity of the gene product thereof is the activity of glutaredoxin.

Accordingly, in one embodiment, the process of the present invention for producing succinate in a non-human organism, like a microorganism or a plant or a part thereof, comprises increasing or generating the activity of a gene product with the activity of a gene product conferring the activity of “glutaredoxin”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said GM02LC12622, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said GM02LC12622, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said GM02LC12622, or a functional equivalent or a homolog thereof as depicted in column 8 of Table II, application no. 15, preferably a homolog or functional equivalent as depicted in column 8 of Table II B, application no. 15, and being depicted in the same respective line as said GM02LC12622, and preferably the activity is increased non-targeted, whereby the respective line disclose in column 7 the fine chemical succinate.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 10811, preferably the coding region thereof, conferred the production of or the increase in succinate compared with the wild type control.

The nucleic acid sequence of GM02LC17485 from Glycine max, e.g. as shown in the respective line in column 5 of Table I, application no. 15, is unpublished. And the activity of the gene product thereof is the activity of GM02LC17485-protein.

Accordingly, in one embodiment, the process of the present invention for producing fumarate in a non-human organism, like a microorganism or a plant or a part thereof, comprises increasing or generating the activity of a gene product with the activity of a gene product conferring the activity of “GM02LC17485-protein”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said GM02LC17485, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said GM02LC17485, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said GM02LC17485, or a functional equivalent or a homolog thereof as depicted in column 8 of Table II, application no. 15, preferably a homolog or functional equivalent as depicted in column 8 of Table II B, application no. 15, and being depicted in the same respective line as said GM02LC17485, and preferably the activity is increased non-targeted, whereby the respective line disclose in column 7 the fine chemical fumarate.

Accordingly, in one embodiment, the molecule which activity is to be increased in the process of the invention is the gene product with an activity as a “GM02LC17485-protein”, preferably it is the molecule of section (a) or (b) of this paragraph.

In particular, it was observed that in plants, especially in Arabidopsis thaliana, increasing or generating the activity of a gene product non-targeted with the activity of a “GM02LC17485-protein”, preferably being encoded by a gene comprising the nucleic acid sequence SEQ ID NO. 46405, preferably the coding region thereof, conferred the production of or the increase in fumarate compared with the wild type control.

The nucleic acid sequence of GM02LC21368 from Glycine max, e.g. as shown in the respective line in column 5 of Table I, application no. 15, is unpublished. And the activity of the gene product thereof is the activity of transcription factor.

Accordingly, in one embodiment, the process of the present invention for producing citramalate in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said GM02LC21368, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said GM02LC21368, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said GM02LC21368, or a functional equivalent or a homolog thereof as depicted in column 8 of Table II, application no. 15, preferably a homolog or functional equivalent as depicted in column 8 of Table II B, application no. 15, and being depicted in the same respective line as said GM02LC21368, and preferably the activity is increased non-targeted, whereby the respective line disclose in column 7 the fine chemical citramalate.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 84198, preferably the coding region thereof, conferred the production of or the increase in citramalate compared with the wild type control.

The nucleic acid sequence of GM02LC21368 from Glycine max, e.g. as shown in the respective line in column 5 of Table I, application no. 15, is unpublished. And the activity of the gene product thereof is the activity of transcription factor.

Accordingly, in one embodiment, the process of the present invention for producing fumarate in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said GM02LC21368, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said GM02LC21368, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said GM02LC21368, or a functional equivalent or a homolog thereof as depicted in column 8 of Table II, application no. 15, preferably a homolog or functional equivalent as depicted in column 8 of Table II B, application no. 15, and being depicted in the same respective line as said GM02LC21368, and preferably the activity is increased non-targeted, whereby the respective line disclose in column 7 the fine chemical fumarate.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 84198, preferably the coding region thereof, conferred the production of or the increase in fumarate compared with the wild type control.

The nucleic acid sequence of GM02LC21368 from Glycine max, e.g. as shown in the respective line in column 5 of Table I, application no. 15, is unpublished. And the activity of the gene product thereof is the activity of transcription factor.

Accordingly, in one embodiment, the process of the present invention for producing malate in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said GM02LC21368, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said GM02LC21368, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said GM02LC21368, or a functional equivalent or a homolog thereof as depicted in column 8 of Table II, application no. 15, preferably a homolog or functional equivalent as depicted in column 8 of Table II B, application no. 15, and being depicted in the same respective line as said GM02LC21368, and preferably the activity is increased non-targeted, whereby the respective line disclose in column 7 the fine chemical malate.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 84198, preferably the coding region thereof, conferred the production of or the increase in malate compared with the wild type control.

The nucleic acid sequence of GM02LC5744 from Glycine max, e.g. as shown in the respective line in column 5 of Table I, application no. 15, is unpublished. And the activity of the gene product thereof is the activity of GM02LC5744-protein.

Accordingly, in one embodiment, the process of the present invention for producing citramalate in a non-human organism, like a microorganism or a plant or a part thereof, comprises increasing or generating the activity of a gene product with the activity of a gene product conferring the activity of “GM02LC5744-protein”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said GM02LC5744, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said GM02LC5744, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said GM02LC5744, or a functional equivalent or a homolog thereof as depicted in column 8 of Table II, application no. 15, preferably a homolog or functional equivalent as depicted in column 8 of Table II B, application no. 15, and being depicted in the same respective line as said GM02LC5744, and preferably the activity is increased non-targeted, whereby the respective line disclose in column 7 the fine chemical citramalate.

Accordingly, in one embodiment, the molecule which activity is to be increased in the process of the invention is the gene product with an activity as a “GM02LC5744-protein”, preferably it is the molecule of section (a) or (b) of this paragraph.

In particular, it was observed that in plants, especially in Arabidopsis thaliana, increasing or generating the activity of a gene product non-targeted with the activity of a “GM02LC5744-protein”, preferably being encoded by a gene comprising the nucleic acid sequence SEQ ID NO. 47076, preferably the coding region thereof, conferred the production of or the increase in citramalate compared with the wild type control.

The nucleic acid sequence of GM02LC5744 from Glycine max, e.g. as shown in the respective line in column 5 of Table I, application no. 15, is unpublished. And the activity of the gene product thereof is the activity of GM02LC5744-protein.

Accordingly, in one embodiment, the process of the present invention for producing fumarate in a non-human organism, like a microorganism or a plant or a part thereof, comprises increasing or generating the activity of a gene product with the activity of a gene product conferring the activity of “GM02LC5744-protein”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said GM02LC5744, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said GM02LC5744, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said GM02LC5744, or a functional equivalent or a homolog thereof as depicted in column 8 of Table II, application no. 15, preferably a homolog or functional equivalent as depicted in column 8 of Table II B, application no. 15, and being depicted in the same respective line as said GM02LC5744, and preferably the activity is increased non-targeted, whereby the respective line disclose in column 7 the fine chemical fumarate.

Accordingly, in one embodiment, the molecule which activity is to be increased in the process of the invention is the gene product with an activity as a “GM02LC5744-protein”, preferably it is the molecule of section (a) or (b) of this paragraph.

In particular, it was observed that in plants, especially in Arabidopsis thaliana, increasing or generating the activity of a gene product non-targeted with the activity of a “GM02LC5744-protein”, preferably being encoded by a gene comprising the nucleic acid sequence SEQ ID NO. 47076, preferably the coding region thereof, conferred the production of or the increase in fumarate compared with the wild type control.

The nucleic acid sequence of Sll0170 from Synechocystis sp., e.g. as shown in the respective line in column 5 of Table I, application no. 15, has been published in C. And the activity of the gene product thereof is the activity of heat shock protein.

Accordingly, in one embodiment, the process of the present invention for producing citramalate in a non-human organism, like a microorganism or a plant or a part thereof, comprises increasing or generating the activity of a gene product with the activity of a gene product conferring the activity of “heat shock protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said Sll0170, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Sll0170, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Sll0170, or a functional equivalent or a homolog thereof as depicted in column 8 of Table II, application no. 15, preferably a homolog or functional equivalent as depicted in column 8 of Table II B, application no. 15, and being depicted in the same respective line as said Sll0170, and preferably the activity is increased non-targeted, whereby the respective line disclose in column 7 the fine chemical citramalate.

Accordingly, in one embodiment, the molecule which activity is to be increased in the process of the invention is the gene product with an activity as a “heat shock protein”, preferably it is the molecule of section (a) or (b) of this paragraph.

In particular, it was observed that in plants, especially in Arabidopsis thaliana, increasing or generating the activity of a gene product non-targeted with the activity of a “heat shock protein”, preferably being encoded by a gene comprising the nucleic acid sequence SEQ ID NO. 48138, preferably the coding region thereof, conferred the production of or the increase in citramalate compared with the wild type control.

The nucleic acid sequence of Sll0228 from Synechocystis sp., e.g. as shown in the respective line in column 5 of Table I, application no. 15, has been published in C. And the activity of the gene product thereof is the activity of agmatinase.

Accordingly, in one embodiment, the process of the present invention for producing fumarate in a non-human organism, like a microorganism or a plant or a part thereof, comprises increasing or generating the activity of a gene product with the activity of a gene product conferring the activity of “agmatinase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said Sll0228, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Sll0228, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Sll0228, or a functional equivalent or a homolog thereof as depicted in column 8 of Table II, application no. 15, preferably a homolog or functional equivalent as depicted in column 8 of Table II B, application no. 15, and being depicted in the same respective line as said Sll0228, and preferably the activity is increased plastidic, whereby the respective line disclose in column 7 the fine chemical fumarate.

Accordingly, in one embodiment, the molecule which activity is to be increased in the process of the invention is the gene product with an activity as a “agmatinase”, preferably it is the molecule of section (a) or (b) of this paragraph.

In particular, it was observed that in plants, especially in Arabidopsis thaliana, increasing or generating the activity of a gene product plastidic with the activity of a “agmatinase”, preferably being encoded by a gene comprising the nucleic acid sequence SEQ ID NO. 84265, preferably the coding region thereof, conferred the production of or the increase in fumarate compared with the wild type control.

The nucleic acid sequence of Sll0240 from Synechocystis sp., e.g. as shown in the respective line in column 5 of Table I, application no. 15, has been published in C. And the activity of the gene product thereof is the activity of ABC transporter ATP binding component.

Accordingly, in one embodiment, the process of the present invention for producing malate in a non-human organism, like a microorganism or a plant or a part thereof, comprises increasing or generating the activity of a gene product with the activity of a gene product conferring the activity of “ABC transporter ATP binding component”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said Sll0240, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Sll0240, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Sll0240, or a functional equivalent or a homolog thereof as depicted in column 8 of Table II, application no. 15, preferably a homolog or functional equivalent as depicted in column 8 of Table II B, application no. 15, and being depicted in the same respective line as said Sll0240, and preferably the activity is increased plastidic, whereby the respective line disclose in column 7 the fine chemical malate.

Accordingly, in one embodiment, the molecule which activity is to be increased in the process of the invention is the gene product with an activity as a “ABC transporter ATP binding component”, preferably it is the molecule of section (a) or (b) of this paragraph.

In particular, it was observed that in plants, especially in Arabidopsis thaliana, increasing or generating the activity of a gene product plastidic with the activity of a “ABC transporter ATP binding component”, preferably being encoded by a gene comprising the nucleic acid sequence SEQ ID NO. 84441, preferably the coding region thereof, conferred the production of or the increase in malate compared with the wild type control.

The nucleic acid sequence of Sll0240 from Synechocystis sp., e.g. as shown in the respective line in column 5 of Table I, application no. 15, has been published in C. And the activity of the gene product thereof is the activity of ABC transporter ATP binding component.

Accordingly, in one embodiment, the process of the present invention for producing succinate in a non-human organism, like a microorganism or a plant or a part thereof, comprises increasing or generating the activity of a gene product with the activity of a gene product conferring the activity of “ABC transporter ATP binding component”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said Sll0240, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Sll0240, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Sll0240, or a functional equivalent or a homolog thereof as depicted in column 8 of Table II, application no. 15, preferably a homolog or functional equivalent as depicted in column 8 of Table II B, application no. 15, and being depicted in the same respective line as said Sll0240, and preferably the activity is increased plastidic, whereby the respective line disclose in column 7 the fine chemical succinate.

Accordingly, in one embodiment, the molecule which activity is to be increased in the process of the invention is the gene product with an activity as a “ABC transporter ATP binding component”, preferably it is the molecule of section (a) or (b) of this paragraph.

In particular, it was observed that in plants, especially in Arabidopsis thaliana, increasing or generating the activity of a gene product plastidic with the activity of a “ABC transporter ATP binding component”, preferably being encoded by a gene comprising the nucleic acid sequence SEQ ID NO. 84441, preferably the coding region thereof, conferred the production of or the increase in succinate compared with the wild type control.

The nucleic acid sequence of Sll0816 from Synechocystis sp., e.g. as shown in the respective line in column 5 of Table I, application no. 15, has been published in C. And the activity of the gene product thereof is the activity of oxireductase.

Accordingly, in one embodiment, the process of the present invention for producing fumarate in a non-human organism, like a microorganism or a plant or a part thereof, comprises increasing or generating the activity of a gene product with the activity of a gene product conferring the activity of “oxireductase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said Sll0816, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Sll0816, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Sll0816, or a functional equivalent or a homolog thereof as depicted in column 8 of Table II, application no. 15, preferably a homolog or functional equivalent as depicted in column 8 of Table II B, application no. 15, and being depicted in the same respective line as said Sll0816, and preferably the activity is increased non-targeted, whereby the respective line disclose in column 7 the fine chemical fumarate.

Accordingly, in one embodiment, the molecule which activity is to be increased in the process of the invention is the gene product with an activity as a “oxireductase”, preferably it is the molecule of section (a) or (b) of this paragraph.

In particular, it was observed that in plants, especially in Arabidopsis thaliana, increasing or generating the activity of a gene product non-targeted with the activity of a “oxireductase”, preferably being encoded by a gene comprising the nucleic acid sequence SEQ ID NO. 51198, preferably the coding region thereof, conferred the production of or the increase in fumarate compared with the wild type control.

The nucleic acid sequence of Sll0895 from Synechocystis sp., e.g. as shown in the respective line in column 5 of Table I, application no. 15, has been published in C. And the activity of the gene product thereof is the activity of phosphatase.

Accordingly, in one embodiment, the process of the present invention for producing succinate in a non-human organism, like a microorganism or a plant or a part thereof, comprises increasing or generating the activity of a gene product with the activity of a gene product conferring the activity of “phosphatase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said Sll0895, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Sll0895, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Sll0895, or a functional equivalent or a homolog thereof as depicted in column 8 of Table II, application no. 15, preferably a homolog or functional equivalent as depicted in column 8 of Table II B, application no. 15, and being depicted in the same respective line as said Sll0895, and preferably the activity is increased non-targeted, whereby the respective line disclose in column 7 the fine chemical succinate.

Accordingly, in one embodiment, the molecule which activity is to be increased in the process of the invention is the gene product with an activity as a “phosphatase”, preferably it is the molecule of section (a) or (b) of this paragraph.

In particular, it was observed that in plants, especially in Arabidopsis thaliana, increasing or generating the activity of a gene product non-targeted with the activity of a “phosphatase”, preferably being encoded by a gene comprising the nucleic acid sequence SEQ ID NO. 127109, preferably the coding region thereof, conferred the production of or the increase in succinate compared with the wild type control.

The nucleic acid sequence of Sll1522 from Synechocystis sp., e.g. as shown in the respective line in column 5 of Table I, application no. 15, has been published in C. And the activity of the gene product thereof is the activity of CDP-diacylglycerol-glycerol-3-phosphate 3-phosphatidyltransferase.

Accordingly, in one embodiment, the process of the present invention for producing citramalate in a non-human organism, like a microorganism or a plant or a part thereof, comprises increasing or generating the activity of a gene product with the activity of a gene product conferring the activity of “CDP-diacylglycerol-glycerol-3-phosphate 3-phosphatidyltransferase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said Sll1522, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Sll1522, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Sll1522, or a functional equivalent or a homolog thereof as depicted in column 8 of Table II, application no. 15, preferably a homolog or functional equivalent as depicted in column 8 of Table II B, application no. 15, and being depicted in the same respective line as said Sll1522, and preferably the activity is increased plastidic, whereby the respective line disclose in column 7 the fine chemical citramalate.

Accordingly, in one embodiment, the molecule which activity is to be increased in the process of the invention is the gene product with an activity as a “CDP-diacylglycerol-glycerol-3-phosphate 3-phosphatidyltransferase”, preferably it is the molecule of section (a) or (b) of this paragraph.

In particular, it was observed that in plants, especially in Arabidopsis thaliana, increasing or generating the activity of a gene product plastidic with the activity of a “CDP-diacylglycerolglycerol-3-phosphate 3-phosphatidyltransferase”, preferably being encoded by a gene comprising the nucleic acid sequence SEQ ID NO. 54452, preferably the coding region thereof, conferred the production of or the increase in citramalate compared with the wild type control.

The nucleic acid sequence of Sll1522 from Synechocystis sp., e.g. as shown in the respective line in column 5 of Table I, application no. 15, has been published in C. And the activity of the gene product thereof is the activity of CDP-diacylglycerol-glycerol-3-phosphate 3-phosphatidyltransferase.

Accordingly, in one embodiment, the process of the present invention for producing malate in a non-human organism, like a microorganism or a plant or a part thereof, comprises increasing or generating the activity of a gene product with the activity of a gene product conferring the activity of “CDP-diacylglycerol-glycerol-3-phosphate 3-phosphatidyltransferase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said Sll1522, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Sll1522, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Sll1522, or a functional equivalent or a homolog thereof as depicted in column 8 of Table II, application no. 15, preferably a homolog or functional equivalent as depicted in column 8 of Table II B, application no. 15, and being depicted in the same respective line as said Sll1522, and preferably the activity is increased plastidic, whereby the respective line disclose in column 7 the fine chemical malate.

Accordingly, in one embodiment, the molecule which activity is to be increased in the process of the invention is the gene product with an activity as a “CDP-diacylglycerol-glycerol-3-phosphate 3-phosphatidyltransferase”, preferably it is the molecule of section (a) or (b) of this paragraph.

In particular, it was observed that in plants, especially in Arabidopsis thaliana, increasing or generating the activity of a gene product plastidic with the activity of a “CDP-diacylglycerolglycerol-3-phosphate 3-phosphatidyltransferase”, preferably being encoded by a gene comprising the nucleic acid sequence SEQ ID NO. 54452, preferably the coding region thereof, conferred the production of or the increase in malate compared with the wild type control.

The nucleic acid sequence of Sll1815 from Synechocystis sp., e.g. as shown in the respective line in column 5 of Table I, application no. 15, has been published in C. And the activity of the gene product thereof is the activity of adenylate kinase.

Accordingly, in one embodiment, the process of the present invention for producing succinate in a non-human organism, like a microorganism or a plant or a part thereof, comprises increasing or generating the activity of a gene product with the activity of a gene product conferring the activity of “adenylate kinase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said Sll1815, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Sll1815, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Sll1815, or a functional equivalent or a homolog thereof as depicted in column 8 of Table II, application no. 15, preferably a homolog or functional equivalent as depicted in column 8 of Table II B, application no. 15, and being depicted in the same respective line as said Sll1815, and preferably the activity is increased mitochondrial, whereby the respective line disclose in column 7 the fine chemical succinate.

Accordingly, in one embodiment, the molecule which activity is to be increased in the process of the invention is the gene product with an activity as a “adenylate kinase”, preferably it is the molecule of section (a) or (b) of this paragraph.

In particular, it was observed that in plants, especially in Arabidopsis thaliana, increasing or generating the activity of a gene product mitochondrial with the activity of a “adenylate kinase”, preferably being encoded by a gene comprising the nucleic acid sequence SEQ ID NO. 85808, preferably the coding region thereof, conferred the production of or the increase in succinate compared with the wild type control.

The nucleic acid sequence of Sll1920 from Synechocystis sp., e.g. as shown in the respective line in column 5 of Table I, application no. 15, has been published in C. And the activity of the gene product thereof is the activity of cation-transporting ATPase.

Accordingly, in one embodiment, the process of the present invention for producing citramalate in a non-human organism, like a microorganism or a plant or a part thereof, comprises increasing or generating the activity of a gene product with the activity of a gene product conferring the activity of “cation-transporting ATPase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said Sll1920, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Sll1920, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Sll1920, or a functional equivalent or a homolog thereof as depicted in column 8 of Table II, application no. 15, preferably a homolog or functional equivalent as depicted in column 8 of Table II B, application no. 15, and being depicted in the same respective line as said Sll1920, and preferably the activity is increased non-targeted, whereby the respective line disclose in column 7 the fine chemical citramalate.

Accordingly, in one embodiment, the molecule which activity is to be increased in the process of the invention is the gene product with an activity as a “cation-transporting ATPase”, preferably it is the molecule of section (a) or (b) of this paragraph.

In particular, it was observed that in plants, especially in Arabidopsis thaliana, increasing or generating the activity of a gene product non-targeted with the activity of a “cation-transporting

ATPase”, preferably being encoded by a gene comprising the nucleic acid sequence SEQ ID NO. 55385, preferably the coding region thereof, conferred the production of or the increase in citramalate compared with the wild type control.

The nucleic acid sequence of Sll1920 from Synechocystis sp., e.g. as shown in the respective line in column 5 of Table I, application no. 15, has been published in C. And the activity of the gene product thereof is the activity of cation-transporting ATPase.

Accordingly, in one embodiment, the process of the present invention for producing malate in a non-human organism, like a microorganism or a plant or a part thereof, comprises increasing or generating the activity of a gene product with the activity of a gene product conferring the activity of “cation-transporting ATPase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said Sll1920, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Sll1920, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Sll1920, or a functional equivalent or a homolog thereof as depicted in column 8 of Table II, application no. 15, preferably a homolog or functional equivalent as depicted in column 8 of Table II B, application no. 15, and being depicted in the same respective line as said Sll1920, and preferably the activity is increased mitochondrial, whereby the respective line disclose in column 7 the fine chemical malate.

Accordingly, in one embodiment, the molecule which activity is to be increased in the process of the invention is the gene product with an activity as a “cation-transporting ATPase”, preferably it is the molecule of section (a) or (b) of this paragraph.

In particular, it was observed that in plants, especially in Arabidopsis thaliana, increasing or generating the activity of a gene product mitochondrial with the activity of a “cation-transporting ATPase”, preferably being encoded by a gene comprising the nucleic acid sequence SEQ ID NO. 55385, preferably the coding region thereof, conferred the production of or the increase in malate compared with the wild type control.

The nucleic acid sequence of Slr0018 from Synechocystis sp., e.g. as shown in the respective line in column 5 of Table I, application no. 15, has been published in C. And the activity of the gene product thereof is the activity of fumarate hydratase.

Accordingly, in one embodiment, the process of the present invention for producing fumarate in a non-human organism, like a microorganism or a plant or a part thereof, comprises increasing or generating the activity of a gene product with the activity of a gene product conferring the activity of “fumarate hydratase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said Slr0018, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Slr0018, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Slr0018, or a functional equivalent or a homolog thereof as depicted in column 8 of Table II, application no. 15, preferably a homolog or functional equivalent as depicted in column 8 of Table II B, application no. 15, and being depicted in the same respective line as said Slr0018, and preferably the activity is increased plastidic, whereby the respective line disclose in column 7 the fine chemical fumarate.

Accordingly, in one embodiment, the molecule which activity is to be increased in the process of the invention is the gene product with an activity as a “fumarate hydratase”, preferably it is the molecule of section (a) or (b) of this paragraph.

In particular, it was observed that in plants, especially in Arabidopsis thaliana, increasing or generating the activity of a gene product plastidic with the activity of a “fumarate hydratase”, preferably being encoded by a gene comprising the nucleic acid sequence SEQ ID NO. 136533, preferably the coding region thereof, conferred the production of or the increase in fumarate compared with the wild type control.

The nucleic acid sequence of Slr0338 from Synechocystis sp., e.g. as shown in the respective line in column 5 of Table I, application no. 15, has been published in C. And the activity of the gene product thereof is the activity of oxidoreductase.

Accordingly, in one embodiment, the process of the present invention for producing citramalate in a non-human organism, like a microorganism or a plant or a part thereof, comprises increasing or generating the activity of a gene product with the activity of a gene product conferring the activity of “oxidoreductase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said Slr0338, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Slr0338, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Slr0338, or a functional equivalent or a homolog thereof as depicted in column 8 of Table II, application no. 15, preferably a homolog or functional equivalent as depicted in column 8 of Table II B, application no. 15, and being depicted in the same respective line as said Slr0338, and preferably the activity is increased plastidic, whereby the respective line disclose in column 7 the fine chemical citramalate.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product plastidic with the activity of a “oxidoreductase”, preferably being encoded by a gene comprising the nucleic acid sequence SEQ ID NO. 11990, preferably the coding region thereof, conferred the production of or the increase in citramalate compared with the wild type control.

The nucleic acid sequence of Slr0597 from Synechocystis sp., e.g. as shown in the respective line in column 5 of Table I, application no. 15, has been published in C. And the activity of the gene product thereof is the activity of bifunctional purine biosynthesis protein.

Accordingly, in one embodiment, the process of the present invention for producing fumarate in a non-human organism, like a microorganism or a plant or a part thereof, comprises increasing or generating the activity of a gene product with the activity of a gene product conferring the activity of “bifunctional purine biosynthesis protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said Slr0597, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Slr0597, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Slr0597, or a functional equivalent or a homolog thereof as depicted in column 8 of Table II, application no. 15, preferably a homolog or functional equivalent as depicted in column 8 of Table II B, application no. 15, and being depicted in the same respective line as said Slr0597, and preferably the activity is increased plastidic, whereby the respective line disclose in column 7 the fine chemical fumarate.

Accordingly, in one embodiment, the molecule which activity is to be increased in the process of the invention is the gene product with an activity as a “bifunctional purine biosynthesis protein”, preferably it is the molecule of section (a) or (b) of this paragraph.

In particular, it was observed that in plants, especially in Arabidopsis thaliana, increasing or generating the activity of a gene product plastidic with the activity of a “bifunctional purine biosynthesis protein”, preferably being encoded by a gene comprising the nucleic acid sequence SEQ ID NO. 56153, preferably the coding region thereof, conferred the production of or the increase in fumarate compared with the wild type control.

The nucleic acid sequence of Slr0721 from Synechocystis sp., e.g. as shown in the respective line in column 5 of Table I, application no. 15, has been published in C. And the activity of the gene product thereof is the activity of malate dehydrogenase.

Accordingly, in one embodiment, the process of the present invention for producing citramalate in a non-human organism, like a microorganism or a plant or a part thereof, comprises increasing or generating the activity of a gene product with the activity of a gene product conferring the activity of “malate dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said Slr0721, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Slr0721, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Slr0721, or a functional equivalent or a homolog thereof as depicted in column 8 of Table II, application no. 15, preferably a homolog or functional equivalent as depicted in column 8 of Table II B, application no. 15, and being depicted in the same respective line as said Slr0721, and preferably the activity is increased plastidic, whereby the respective line disclose in column 7 the fine chemical citramalate.

Accordingly, in one embodiment, the molecule which activity is to be increased in the process of the invention is the gene product with an activity as a “malate dehydrogenase”, preferably it is the molecule of section (a) or (b) of this paragraph.

In particular, it was observed that in plants, especially in Arabidopsis thaliana, increasing or generating the activity of a gene product plastidic with the activity of a “malate dehydrogenase”, preferably being encoded by a gene comprising the nucleic acid sequence SEQ ID NO. 87397, preferably the coding region thereof, conferred the production of or the increase in citramalate compared with the wild type control.

The nucleic acid sequence of Slr0721 from Synechocystis sp., e.g. as shown in the respective line in column 5 of Table I, application no. 15, has been published in C. And the activity of the gene product thereof is the activity of malate dehydrogenase.

Accordingly, in one embodiment, the process of the present invention for producing succinate in a non-human organism, like a microorganism or a plant or a part thereof, comprises increasing or generating the activity of a gene product with the activity of a gene product conferring the activity of “malate dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said Slr0721, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Slr0721, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Slr0721, or a functional equivalent or a homolog thereof as depicted in column 8 of Table II, application no. 15, preferably a homolog or functional equivalent as depicted in column 8 of Table II B, application no. 15, and being depicted in the same respective line as said Slr0721, and preferably the activity is increased plastidic, whereby the respective line disclose in column 7 the fine chemical succinate.

Accordingly, in one embodiment, the molecule which activity is to be increased in the process of the invention is the gene product with an activity as a “malate dehydrogenase”, preferably it is the molecule of section (a) or (b) of this paragraph.

In particular, it was observed that in plants, especially in Arabidopsis thaliana, increasing or generating the activity of a gene product plastidic with the activity of a “malate dehydrogenase”, preferably being encoded by a gene comprising the nucleic acid sequence SEQ ID NO. 87397, preferably the coding region thereof, conferred the production of or the increase in succinate compared with the wild type control.

The nucleic acid sequence of Slr1096 from Synechocystis sp., e.g. as shown in the respective line in column 5 of Table I, application no. 15, has been published in C. And the activity of the gene product thereof is the activity of dihydrolipoamide dehydrogenase.

Accordingly, in one embodiment, the process of the present invention for producing glycerate in a non-human organism, like a microorganism or a plant or a part thereof, comprises increasing or generating the activity of a gene product with the activity of a gene product conferring the activity of “dihydrolipoamide dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glycerate), application no. 15, preferably the coding region thereof, or a homolog or a fragment thereof, and being depicted in the same respective line as said Slr1096, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Slr1096, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Slr1096, or a functional equivalent or a homolog thereof as depicted in column 8 of Table II, application no. 15, preferably a homolog or functional equivalent as depicted in column 8 of Table II B, application no. 15, and being depicted in the same respective line as said Slr1096, and preferably the activity is increased non-targeted, whereby the respective line disclose in column 7 the fine chemical glycerate.

Accordingly, in one embodiment, the molecule which activity is to be increased in the process of the invention is the gene product with an activity as a “dihydrolipoamide dehydrogenase”, preferably it is the molecule of section (a) or (b) of this paragraph.

In particular, it was observed that in plants, especially in Arabidopsis thaliana, increasing or generating the activity of a gene product non-targeted with the activity of a “dihydrolipoamide dehydrogenase”, preferably being encoded by a gene comprising the nucleic acid sequence SEQ ID NO. 57734, preferably the coding region thereof, conferred the production of or the increase in glycerate compared with the wild type control.

The nucleic acid sequence of Slr1096 from Synechocystis sp., e.g. as shown in the respective line in column 5 of Table I, application no. 15, has been published in C. And the activity of the gene product thereof is the activity of dihydrolipoamide dehydrogenase.

Accordingly, in one embodiment, the process of the present invention for producing succinate in a non-human organism, like a microorganism or a plant or a part thereof, comprises increasing or generating the activity of a gene product with the activity of a gene product conferring the activity of “dihydrolipoamide dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said Slr1096, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Slr1096, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Slr1096, or a functional equivalent or a homolog thereof as depicted in column 8 of Table II, application no. 15, preferably a homolog or functional equivalent as depicted in column 8 of Table II B, application no. 15, and being depicted in the same respective line as said Slr1096, and preferably the activity is increased non-targeted, whereby the respective line disclose in column 7 the fine chemical succinate.

Accordingly, in one embodiment, the molecule which activity is to be increased in the process of the invention is the gene product with an activity as a “dihydrolipoamide dehydrogenase”, preferably it is the molecule of section (a) or (b) of this paragraph.

In particular, it was observed that in plants, especially in Arabidopsis thaliana, increasing or generating the activity of a gene product non-targeted with the activity of a “dihydrolipoamide dehydrogenase”, preferably being encoded by a gene comprising the nucleic acid sequence SEQ ID NO. 57734, preferably the coding region thereof, conferred the production of or the increase in succinate compared with the wild type control.

The nucleic acid sequence of Slr1170 from Synechocystis sp., e.g. as shown in the respective line in column 5 of Table I, application no. 15, has been published in C. And the activity of the gene product thereof is the activity of slr1170-protein.

Accordingly, in one embodiment, the process of the present invention for producing succinate in a non-human organism, like a microorganism or a plant or a part thereof, comprises increasing or generating the activity of a gene product with the activity of a gene product conferring the activity of “s1r1170-protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said Slr1170, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Slr1170, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Slr1170, or a functional equivalent or a homolog thereof as depicted in column 8 of Table II, application no. 15, preferably a homolog or functional equivalent as depicted in column 8 of Table II B, application no. 15, and being depicted in the same respective line as said Slr1170, and preferably the activity is increased non-targeted, whereby the respective line disclose in column 7 the fine chemical succinate.

Accordingly, in one embodiment, the molecule which activity is to be increased in the process of the invention is the gene product with an activity as a “slr1170-protein”, preferably it is the molecule of section (a) or (b) of this paragraph.

In particular, it was observed that in plants, especially in Arabidopsis thaliana, increasing or generating the activity of a gene product non-targeted with the activity of a “slr1170-protein”, preferably being encoded by a gene comprising the nucleic acid sequence SEQ ID NO. 136869, preferably the coding region thereof, conferred the production of or the increase in succinate compared with the wild type control.

The nucleic acid sequence of Slr1250 from Synechocystis sp., e.g. as shown in the respective line in column 5 of Table I, application no. 15, has been published in C. And the activity of the gene product thereof is the activity of phosphate import ATP-binding protein.

Accordingly, in one embodiment, the process of the present invention for producing fumarate in a non-human organism, like a microorganism or a plant or a part thereof, comprises increasing or generating the activity of a gene product with the activity of a gene product conferring the activity of “phosphate import ATP-binding protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said Slr1250, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Slr1250, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Slr1250, or a functional equivalent or a homolog thereof as depicted in column 8 of Table II, application no. 15, preferably a homolog or functional equivalent as depicted in column 8 of Table II B, application no. 15, and being depicted in the same respective line as said Slr1250, and preferably the activity is increased mitochondrial, whereby the respective line disclose in column 7 the fine chemical fumarate.

Accordingly, in one embodiment, the molecule which activity is to be increased in the process of the invention is the gene product with an activity as a “phosphate import ATP-binding protein”, preferably it is the molecule of section (a) or (b) of this paragraph.

In particular, it was observed that in plants, especially in Arabidopsis thaliana, increasing or generating the activity of a gene product mitochondrial with the activity of a “phosphate import ATP-binding protein”, preferably being encoded by a gene comprising the nucleic acid sequence SEQ ID NO. 116460, preferably the coding region thereof, conferred the production of or the increase in fumarate compared with the wild type control.

The nucleic acid sequence of Slr1269 from Synechocystis sp., e.g. as shown in the respective line in column 5 of Table I, application no. 15, has been published in C. And the activity of the gene product thereof is the activity of gamma-glutamyltranspeptidase.

Accordingly, in one embodiment, the process of the present invention for producing succinate in a non-human organism, like a microorganism or a plant or a part thereof, comprises increasing or generating the activity of a gene product with the activity of a gene product conferring the activity of “gamma-glutamyltranspeptidase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said Slr1269, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Slr1269, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Slr1269, or a functional equivalent or a homolog thereof as depicted in column 8 of Table II, application no. 15, preferably a homolog or functional equivalent as depicted in column 8 of Table II B, application no. 15, and being depicted in the same respective line as said Slr1269, and preferably the activity is increased non-targeted, whereby the respective line disclose in column 7 the fine chemical succinate.

Accordingly, in one embodiment, the molecule which activity is to be increased in the process of the invention is the gene product with an activity as a “gamma-glutamyltranspeptidase”, preferably it is the molecule of section (a) or (b) of this paragraph.

In particular, it was observed that in plants, especially in Arabidopsis thaliana, increasing or generating the activity of a gene product non-targeted with the activity of a “gamma-glutamyltranspeptidase”, preferably being encoded by a gene comprising the nucleic acid sequence SEQ ID NO. 58058, preferably the coding region thereof, conferred the production of or the increase in succinate compared with the wild type control.

The nucleic acid sequence of Slr1739 from Synechocystis sp., e.g. as shown in the respective line in column 5 of Table I, application no. 15, has been published in C. And the activity of the gene product thereof is the activity of photosystem II protein.

Accordingly, in one embodiment, the process of the present invention for producing malate in a non-human organism, like a microorganism or a plant or a part thereof, comprises increasing or generating the activity of a gene product with the activity of a gene product conferring the activity of “photosystem II protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said Slr1739, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Slr1739, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Slr1739, or a functional equivalent or a homolog thereof as depicted in column 8 of Table II, application no. 15, preferably a homolog or functional equivalent as depicted in column 8 of Table II B, application no. 15, and being depicted in the same respective line as said Slr1739, and preferably the activity is increased plastidic, whereby the respective line disclose in column 7 the fine chemical malate.

Accordingly, in one embodiment, the molecule which activity is to be increased in the process of the invention is the gene product with an activity as a “photosystem II protein”, preferably it is the molecule of section (a) or (b) of this paragraph.

In particular, it was observed that in plants, especially in Arabidopsis thaliana, increasing or generating the activity of a gene product plastidic with the activity of a “photosystem II protein”, preferably being encoded by a gene comprising the nucleic acid sequence SEQ ID NO. 58731, preferably the coding region thereof, conferred the production of or the increase in malate compared with the wild type control.

The nucleic acid sequence of Slr2023 from Synechocystis sp., e.g. as shown in the respective line in column 5 of Table I, application no. 15, has been published in C. And the activity of the gene product thereof is the activity of malonyl CoA-acyl carrier protein transacylase.

Accordingly, in one embodiment, the process of the present invention for producing fumarate in a non-human organism, like a microorganism or a plant or a part thereof, comprises increasing or generating the activity of a gene product with the activity of a gene product conferring the activity of “malonyl CoA-acyl carrier protein transacylase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said Slr2023, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Slr2023, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Slr2023, or a functional equivalent or a homolog thereof as depicted in column 8 of Table II, application no. 15, preferably a homolog or functional equivalent as depicted in column 8 of Table II B, application no. 15, and being depicted in the same respective line as said Slr2023, and preferably the activity is increased plastidic, whereby the respective line disclose in column 7 the fine chemical fumarate.

Accordingly, in one embodiment, the molecule which activity is to be increased in the process of the invention is the gene product with an activity as a “malonyl CoA-acyl carrier protein transacylase”, preferably it is the molecule of section (a) or (b) of this paragraph.

In particular, it was observed that in plants, especially in Arabidopsis thaliana, increasing or generating the activity of a gene product plastidic with the activity of a “malonyl CoA-acyl carrier protein transacylase”, preferably being encoded by a gene comprising the nucleic acid sequence SEQ ID NO. 59165, preferably the coding region thereof, conferred the production of or the increase in fumarate compared with the wild type control.

The nucleic acid sequence of Slr2023 from Synechocystis sp., e.g. as shown in the respective line in column 5 of Table I, application no. 15, has been published in C. And the activity of the gene product thereof is the activity of malonyl CoA-acyl carrier protein transacylase.

Accordingly, in one embodiment, the process of the present invention for producing succinate in a non-human organism, like a microorganism or a plant or a part thereof, comprises increasing or generating the activity of a gene product with the activity of a gene product conferring the activity of “malonyl CoA-acyl carrier protein transacylase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said

Slr2023, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Slr2023, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Slr2023, or a functional equivalent or a homolog thereof as depicted in column 8 of Table II, application no. 15, preferably a homolog or functional equivalent as depicted in column 8 of Table II B, application no. 15, and being depicted in the same respective line as said Slr2023, and preferably the activity is increased plastidic, whereby the respective line disclose in column 7 the fine chemical succinate.

Accordingly, in one embodiment, the molecule which activity is to be increased in the process of the invention is the gene product with an activity as a “malonyl CoA-acyl carrier protein transacylase”, preferably it is the molecule of section (a) or (b) of this paragraph.

In particular, it was observed that in plants, especially in Arabidopsis thaliana, increasing or generating the activity of a gene product plastidic with the activity of a “malonyl CoA-acyl carrier protein transacylase”, preferably being encoded by a gene comprising the nucleic acid sequence SEQ ID NO. 59165, preferably the coding region thereof, conferred the production of or the increase in succinate compared with the wild type control.

The nucleic acid sequence of Smr0004 from Synechocystis sp., e.g. as shown in the respective line in column 5 of Table I, application no. 15, has been published in C. And the activity of the gene product thereof is the activity of photosystem I reaction centre subunit.

Accordingly, in one embodiment, the process of the present invention for producing glycerate in a non-human organism, like a microorganism or a plant or a part thereof, comprises increasing or generating the activity of a gene product with the activity of a gene product conferring the activity of “photosystem I reaction centre subunit”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glycerate), application no. 15, preferably the coding region thereof, or a homolog or a fragment thereof, and being depicted in the same respective line as said Smr0004, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Smr0004, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Smr0004, or a functional equivalent or a homolog thereof as depicted in column 8 of Table II, application no. 15, preferably a homolog or functional equivalent as depicted in column 8 of Table II B, application no. 15, and being depicted in the same respective line as said Smr0004, and preferably the activity is increased plastidic, whereby the respective line disclose in column 7 the fine chemical glycerate.

Accordingly, in one embodiment, the molecule which activity is to be increased in the process of the invention is the gene product with an activity as a “photosystem I reaction centre subunit”, preferably it is the molecule of section (a) or (b) of this paragraph.

In particular, it was observed that in plants, especially in Arabidopsis thaliana, increasing or generating the activity of a gene product plastidic with the activity of a “photosystem I reaction centre subunit”, preferably being encoded by a gene comprising the nucleic acid sequence SEQ ID NO. 136907, preferably the coding region thereof, conferred the production of or the increase in glycerate compared with the wild type control.

The nucleic acid sequence of TTC0019 from Thermus thermophilus, e.g. as shown in the respective line in column 5 of Table I, application no. 15, has been published in E. And the activity of the gene product thereof is the activity of Sec-independent protein translocase subunit.

Accordingly, in one embodiment, the process of the present invention for producing fumarate in a non-human organism, like a microorganism or a plant or a part thereof, comprises increasing or generating the activity of a gene product with the activity of a gene product conferring the activity of “Sec-independent protein translocase subunit”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said TTC0019, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said TTC0019, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said TTC0019, or a functional equivalent or a homolog thereof as depicted in column 8 of Table II, application no. 15, preferably a homolog or functional equivalent as depicted in column 8 of Table II B, application no. 15, and being depicted in the same respective line as said TTC0019, and preferably the activity is increased non-targeted, whereby the respective line disclose in column 7 the fine chemical fumarate.

Accordingly, in one embodiment, the molecule which activity is to be increased in the process of the invention is the gene product with an activity as a “Sec-independent protein translocase subunit”, preferably it is the molecule of section (a) or (b) of this paragraph.

In particular, it was observed that in plants, especially in Arabidopsis thaliana, increasing or generating the activity of a gene product non-targeted with the activity of a “Sec-independent protein translocase subunit”, preferably being encoded by a gene comprising the nucleic acid sequence SEQ ID NO. 12698, preferably the coding region thereof, conferred the production of or the increase in fumarate compared with the wild type control.

The nucleic acid sequence of TTC0019 from Thermus thermophilus, e.g. as shown in the respective line in column 5 of Table I, application no. 15, has been published in E. And the activity of the gene product thereof is the activity of Sec-independent protein translocase subunit.

Accordingly, in one embodiment, the process of the present invention for producing succinate in a non-human organism, like a microorganism or a plant or a part thereof, comprises increasing or generating the activity of a gene product with the activity of a gene product conferring the activity of “Sec-independent protein translocase subunit”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said TTC0019, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said TTC0019, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said TTC0019, or a functional equivalent or a homolog thereof as depicted in column 8 of Table II, application no. 15, preferably a homolog or functional equivalent as depicted in column 8 of Table II B, application no. 15, and being depicted in the same respective line as said TTC0019, and preferably the activity is increased non-targeted, whereby the respective line disclose in column 7 the fine chemical succinate.

Accordingly, in one embodiment, the molecule which activity is to be increased in the process of the invention is the gene product with an activity as a “Sec-independent protein translocase subunit”, preferably it is the molecule of section (a) or (b) of this paragraph.

In particular, it was observed that in plants, especially in Arabidopsis thaliana, increasing or generating the activity of a gene product non-targeted with the activity of a “Sec-independent protein translocase subunit”, preferably being encoded by a gene comprising the nucleic acid sequence SEQ ID NO. 12698, preferably the coding region thereof, conferred the production of or the increase in succinate compared with the wild type control.

The nucleic acid sequence of TTC0849 from Thermus thermophilus, e.g. as shown in the respective line in column 5 of Table I, application no. 15, has been published in E. And the activity of the gene product thereof is the activity of 2-isopropylmalate synthase.

Accordingly, in one embodiment, the process of the present invention for producing citramalate in a non-human organism, like a microorganism or a plant or a part thereof, comprises increasing or generating the activity of a gene product with the activity of a gene product conferring the activity of “2-isopropylmalate synthase”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said TTC0849, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said TTC0849, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said TTC0849, or a functional equivalent or a homolog thereof as depicted in column 8 of Table II, application no. 15, preferably a homolog or functional equivalent as depicted in column 8 of Table II B, application no. 15, and being depicted in the same respective line as said TTC0849, and preferably the activity is increased non-targeted, whereby the respective line disclose in column 7 the fine chemical citramalate.

Accordingly, in one embodiment, the molecule which activity is to be increased in the process of the invention is the gene product with an activity as a “2-isopropylmalate synthase”, preferably it is the molecule of section (a) or (b) of this paragraph.

In particular, it was observed that in plants, especially in Arabidopsis thaliana, increasing or generating the activity of a gene product non-targeted with the activity of a “2-isopropylmalate synthase”, preferably being encoded by a gene comprising the nucleic acid sequence SEQ ID NO. 107556, preferably the coding region thereof, conferred the production of or the increase in citramalate compared with the wild type control.

The nucleic acid sequence of TTC1193 from Thermus thermophilus, e.g. as shown in the respective line in column 5 of Table I, application no. 15, has been published in E. And the activity of the gene product thereof is the activity of multiple antibiotic resistance protein.

Accordingly, in one embodiment, the process of the present invention for producing fumarate in a non-human organism, like a microorganism or a plant or a part thereof, comprises increasing or generating the activity of a gene product with the activity of a gene product conferring the activity of “multiple antibiotic resistance protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said TTC1193, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said TTC1193, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said TTC1193, or a functional equivalent or a homolog thereof as depicted in column 8 of Table II, application no. 15, preferably a homolog or functional equivalent as depicted in column 8 of Table II B, application no. 15, and being depicted in the same respective line as said TTC1193, and preferably the activity is increased non-targeted, whereby the respective line disclose in column 7 the fine chemical fumarate.

Accordingly, in one embodiment, the molecule which activity is to be increased in the process of the invention is the gene product with an activity as a “multiple antibiotic resistance protein”, preferably it is the molecule of section (a) or (b) of this paragraph.

In particular, it was observed that in plants, especially in Arabidopsis thaliana, increasing or generating the activity of a gene product non-targeted with the activity of a “multiple antibiotic resistance protein”, preferably being encoded by a gene comprising the nucleic acid sequence SEQ ID NO. 61723, preferably the coding region thereof, conferred the production of or the increase in fumarate compared with the wild type control.

The nucleic acid sequence of TTC1193 from Thermus thermophilus, e.g. as shown in the respective line in column 5 of Table I, application no. 15, has been published in E. And the activity of the gene product thereof is the activity of multiple antibiotic resistance protein.

Accordingly, in one embodiment, the process of the present invention for producing succinate in a non-human organism, like a microorganism or a plant or a part thereof, comprises increasing or generating the activity of a gene product with the activity of a gene product conferring the activity of “multiple antibiotic resistance protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said TTC1193, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said TTC1193, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said TTC1193, or a functional equivalent or a homolog thereof as depicted in column 8 of Table II, application no. 15, preferably a homolog or functional equivalent as depicted in column 8 of Table II B, application no. 15, and being depicted in the same respective line as said TTC1193, and preferably the activity is increased non-targeted, whereby the respective line disclose in column 7 the fine chemical succinate.

Accordingly, in one embodiment, the molecule which activity is to be increased in the process of the invention is the gene product with an activity as a “multiple antibiotic resistance protein”, preferably it is the molecule of section (a) or (b) of this paragraph.

In particular, it was observed that in plants, especially in Arabidopsis thaliana, increasing or generating the activity of a gene product non-targeted with the activity of a “multiple antibiotic resistance protein”, preferably being encoded by a gene comprising the nucleic acid sequence SEQ ID NO. 61723, preferably the coding region thereof, conferred the production of or the increase in succinate compared with the wild type control.

The nucleic acid sequence of TTC1386 from Thermus thermophilus, e.g. as shown in the respective line in column 5 of Table I, application no. 15, has been published in E. And the activity of the gene product thereof is the activity of TTC1386-protein.

Accordingly, in one embodiment, the process of the present invention for producing fumarate in a non-human organism, like a microorganism or a plant or a part thereof, comprises increasing or generating the activity of a gene product with the activity of a gene product conferring the activity of “TTC1386-protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said TTC1386, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said TTC1386, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said TTC1386, or a functional equivalent or a homolog thereof as depicted in column 8 of Table II, application no. 15, preferably a homolog or functional equivalent as depicted in column 8 of Table II B, application no. 15, and being depicted in the same respective line as said TTC1386, and preferably the activity is increased non-targeted, whereby the respective line disclose in column 7 the fine chemical fumarate.

Accordingly, in one embodiment, the molecule which activity is to be increased in the process of the invention is the gene product with an activity as a “TTC1386-protein”, preferably it is the molecule of section (a) or (b) of this paragraph.

In particular, it was observed that in plants, especially in Arabidopsis thaliana, increasing or generating the activity of a gene product non-targeted with the activity of a “TTC1386-protein”, preferably being encoded by a gene comprising the nucleic acid sequence SEQ ID NO. 62079, preferably the coding region thereof, conferred the production of or the increase in fumarate compared with the wild type control.

The nucleic acid sequence of TTC1386 from Thermus thermophilus, e.g. as shown in the respective line in column 5 of Table I, application no. 15, has been published in E. And the activity of the gene product thereof is the activity of TTC1386-protein.

Accordingly, in one embodiment, the process of the present invention for producing malate in a non-human organism, like a microorganism or a plant or a part thereof, comprises increasing or generating the activity of a gene product with the activity of a gene product conferring the activity of “TTC1386-protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said TTC1386, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said TTC1386, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said TTC1386, or a functional equivalent or a homolog thereof as depicted in column 8 of Table II, application no. 15, preferably a homolog or functional equivalent as depicted in column 8 of Table II B, application no. 15, and being depicted in the same respective line as said TTC1386, and preferably the activity is increased non-targeted, whereby the respective line disclose in column 7 the fine chemical malate.

Accordingly, in one embodiment, the molecule which activity is to be increased in the process of the invention is the gene product with an activity as a “TTC1386-protein”, preferably it is the molecule of section (a) or (b) of this paragraph.

In particular, it was observed that in plants, especially in Arabidopsis thaliana, increasing or generating the activity of a gene product non-targeted with the activity of a “TTC1386-protein”, preferably being encoded by a gene comprising the nucleic acid sequence SEQ ID NO. 62079, preferably the coding region thereof, conferred the production of or the increase in malate compared with the wild type control.

The nucleic acid sequence of TTC1386 from Thermus thermophilus, e.g. as shown in the respective line in column 5 of Table I, application no. 15, has been published in E. And the activity of the gene product thereof is the activity of TTC1386-protein.

Accordingly, in one embodiment, the process of the present invention for producing succinate in a non-human organism, like a microorganism or a plant or a part thereof, comprises increasing or generating the activity of a gene product with the activity of a gene product conferring the activity of “TTC1386-protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said TTC1386, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said TTC1386, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said TTC1386, or a functional equivalent or a homolog thereof as depicted in column 8 of Table II, application no. 15, preferably a homolog or functional equivalent as depicted in column 8 of Table II B, application no. 15, and being depicted in the same respective line as said TTC1386, and preferably the activity is increased non-targeted, whereby the respective line disclose in column 7 the fine chemical succinate.

Accordingly, in one embodiment, the molecule which activity is to be increased in the process of the invention is the gene product with an activity as a “TTC1386-protein”, preferably it is the molecule of section (a) or (b) of this paragraph.

In particular, it was observed that in plants, especially in Arabidopsis thaliana, increasing or generating the activity of a gene product non-targeted with the activity of a “TTC1386-protein”, preferably being encoded by a gene comprising the nucleic acid sequence SEQ ID NO. 62079, preferably the coding region thereof, conferred the production of or the increase in succinate compared with the wild type control.

The nucleic acid sequence of TTC1918 from Thermus thermophilus, e.g. as shown in the respective line in column 5 of Table I, application no. 15, has been published in E. And the activity of the gene product thereof is the activity of oxidoreductase subunit.

Accordingly, in one embodiment, the process of the present invention for producing citramalate in a non-human organism, like a microorganism or a plant or a part thereof, comprises increasing or generating the activity of a gene product with the activity of a gene product conferring the activity of “oxidoreductase subunit”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said TTC1918, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said TTC1918, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said TTC1918, or a functional equivalent or a homolog thereof as depicted in column 8 of Table II, application no. 15, preferably a homolog or functional equivalent as depicted in column 8 of Table II B, application no. 15, and being depicted in the same respective line as said TTC1918, and preferably the activity is increased non-targeted, whereby the respective line disclose in column 7 the fine chemical citramalate.

Accordingly, in one embodiment, the molecule which activity is to be increased in the process of the invention is the gene product with an activity as a “oxidoreductase subunit”, preferably it is the molecule of section (a) or (b) of this paragraph.

In particular, it was observed that in plants, especially in Arabidopsis thaliana, increasing or generating the activity of a gene product non-targeted with the activity of a “oxidoreductase subunit”, preferably being encoded by a gene comprising the nucleic acid sequence SEQ ID NO. 62160, preferably the coding region thereof, conferred the production of or the increase in citramalate compared with the wild type control.

The nucleic acid sequence of TTC1918 from Thermus thermophilus, e.g. as shown in the respective line in column 5 of Table I, application no. 15, has been published in E. And the activity of the gene product thereof is the activity of oxidoreductase subunit.

Accordingly, in one embodiment, the process of the present invention for producing glycerate in a non-human organism, like a microorganism or a plant or a part thereof, comprises increasing or generating the activity of a gene product with the activity of a gene product conferring the activity of “oxidoreductase subunit”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical glycerate), application no. 15, preferably the coding region thereof, or a homolog or a fragment thereof, and being depicted in the same respective line as said TTC1918, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said TTC1918, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said TTC1918, or a functional equivalent or a homolog thereof as depicted in column 8 of Table II, application no. 15, preferably a homolog or functional equivalent as depicted in column 8 of Table II B, application no. 15, and being depicted in the same respective line as said TTC1918, and preferably the activity is increased non-targeted, whereby the respective line disclose in column 7 the fine chemical glycerate.

Accordingly, in one embodiment, the molecule which activity is to be increased in the process of the invention is the gene product with an activity as a “oxidoreductase subunit”, preferably it is the molecule of section (a) or (b) of this paragraph.

In particular, it was observed that in plants, especially in Arabidopsis thaliana, increasing or generating the activity of a gene product non-targeted with the activity of a “oxidoreductase subunit”, preferably being encoded by a gene comprising the nucleic acid sequence SEQ ID NO. 62160, preferably the coding region thereof, conferred the production of or the increase in glycerate compared with the wild type control.

The nucleic acid sequence of TTC1918 from Thermus thermophilus, e.g. as shown in the respective line in column 5 of Table I, application no. 15, has been published in E. And the activity of the gene product thereof is the activity of oxidoreductase subunit.

Accordingly, in one embodiment, the process of the present invention for producing malate in a non-human organism, like a microorganism or a plant or a part thereof, comprises increasing or generating the activity of a gene product with the activity of a gene product conferring the activity of “oxidoreductase subunit”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said TTC1918, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said TTC1918, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said TTC1918, or a functional equivalent or a homolog thereof as depicted in column 8 of Table II, application no. 15, preferably a homolog or functional equivalent as depicted in column 8 of Table II B, application no. 15, and being depicted in the same respective line as said TTC1918, and preferably the activity is increased non-targeted, whereby the respective line disclose in column 7 the fine chemical malate.

Accordingly, in one embodiment, the molecule which activity is to be increased in the process of the invention is the gene product with an activity as a “oxidoreductase subunit”, preferably it is the molecule of section (a) or (b) of this paragraph.

In particular, it was observed that in plants, especially in Arabidopsis thaliana, increasing or generating the activity of a gene product non-targeted with the activity of a “oxidoreductase subunit”, preferably being encoded by a gene comprising the nucleic acid sequence SEQ ID NO. 62160, preferably the coding region thereof, conferred the production of or the increase in malate compared with the wild type control.

The nucleic acid sequence of XM_{—}473199 from Oryza sativa, e.g. as shown in the respective line in column 5 of Table I, application no. 15, has been published in G. And the activity of the gene product thereof is the activity of XM_{—}473199-protein.

Accordingly, in one embodiment, the process of the present invention for producing fumarate in a non-human organism, like a microorganism or a plant or a part thereof, comprises increasing or generating the activity of a gene product with the activity of a gene product conferring the activity of “XM_{—}473199-protein”, especially from Oryza sativa or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said XM_{—}473199, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said XM_{—}473199, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said XM_{—}473199, or a functional equivalent or a homolog thereof as depicted in column 8 of Table II, application no. 15, preferably a homolog or functional equivalent as depicted in column 8 of Table II B, application no. 15, and being depicted in the same respective line as said XM_{—}473199, and preferably the activity is increased non-targeted, whereby the respective line disclose in column 7 the fine chemical fumarate.

Accordingly, in one embodiment, the molecule which activity is to be increased in the process of the invention is the gene product with an activity as a “XM_{—}473199-protein”, preferably it is the molecule of section (a) or (b) of this paragraph.

In particular, it was observed that in plants, especially in Arabidopsis thaliana, increasing or generating the activity of a gene product non-targeted with the activity of a “XM_{—}473199protein”, preferably being encoded by a gene comprising the nucleic acid sequence SEQ ID NO. 62244, preferably the coding region thereof, conferred the production of or the increase in fumarate compared with the wild type control.

The nucleic acid sequence of XM_{—}473199 from Oryza sativa, e.g. as shown in the respective line in column 5 of Table I, application no. 15, has been published in G. And the activity of the gene product thereof is the activity of XM_{—}473199-protein.

Accordingly, in one embodiment, the process of the present invention for producing malate in a non-human organism, like a microorganism or a plant or a part thereof, comprises increasing or generating the activity of a gene product with the activity of a gene product conferring the activity of “XM_{—}473199-protein”, especially from Oryza sativa or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said XM_{—}473199, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said XM_{—}473199, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said XM_{—}473199, or a functional equivalent or a homolog thereof as depicted in column 8 of Table II, application no. 15, preferably a homolog or functional equivalent as depicted in column 8 of Table II B, application no. 15, and being depicted in the same respective line as said XM_{—}473199, and preferably the activity is increased non-targeted, whereby the respective line disclose in column 7 the fine chemical malate.

Accordingly, in one embodiment, the molecule which activity is to be increased in the process of the invention is the gene product with an activity as a “XM_{—}473199-protein”, preferably it is the molecule of section (a) or (b) of this paragraph.

In particular, it was observed that in plants, especially in Arabidopsis thaliana, increasing or generating the activity of a gene product non-targeted with the activity of a “XM_{—}473199-protein”, preferably being encoded by a gene comprising the nucleic acid sequence SEQ ID NO. 62244, preferably the coding region thereof, conferred the production of or the increase in malate compared with the wild type control.

The nucleic acid sequence of XM_{—}473199 from Oryza sativa, e.g. as shown in the respective line in column 5 of Table I, application no. 15, has been published in G. And the activity of the gene product thereof is the activity of XM_{—}473199-protein.

Accordingly, in one embodiment, the process of the present invention for producing succinate in a non-human organism, like a microorganism or a plant or a part thereof, comprises increasing or generating the activity of a gene product with the activity of a gene product conferring the activity of “XM_{—}473199-protein”, especially from Oryza sativa or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said XM_{—}473199, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said XM_{—}473199, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said XM_{—}473199, or a functional equivalent or a homolog thereof as depicted in column 8 of Table II, application no. 15, preferably a homolog or functional equivalent as depicted in column 8 of Table II B, application no. 15, and being depicted in the same respective line as said XM_{—}473199, and preferably the activity is increased non-targeted, whereby the respective line disclose in column 7 the fine chemical succinate.

Accordingly, in one embodiment, the molecule which activity is to be increased in the process of the invention is the gene product with an activity as a “XM_{—}473199-protein”, preferably it is the molecule of section (a) or (b) of this paragraph.

In particular, it was observed that in plants, especially in Arabidopsis thaliana, increasing or generating the activity of a gene product non-targeted with the activity of a “XM_{—}473199-protein”, preferably being encoded by a gene comprising the nucleic acid sequence SEQ ID NO. 62244, preferably the coding region thereof, conferred the production of or the increase in succinate compared with the wild type control.

The nucleic acid sequence of Yal062w from Saccharomyces cerevisiae, e.g. as shown in the respective line in column 5 of Table I, application no. 15, has been published in A. And the activity of the gene product thereof is the activity of glutamate dehydrogenase.

Accordingly, in one embodiment, the process of the present invention for producing succinate in a non-human organism, like a microorganism or a plant or a part thereof, comprises increasing or generating the activity of a gene product with the activity of a gene product conferring the activity of “glutamate dehydrogenase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said Yal062w, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Yal062w, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Yal062w, or a functional equivalent or a homolog thereof as depicted in column 8 of Table II, application no. 15, preferably a homolog or functional equivalent as depicted in column 8 of Table II B, application no. 15, and being depicted in the same respective line as said Yal062w, and preferably the activity is increased plastidic, whereby the respective line disclose in column 7 the fine chemical succinate.

Accordingly, in one embodiment, the molecule which activity is to be increased in the process of the invention is the gene product with an activity as a “glutamate dehydrogenase”, preferably it is the molecule of section (a) or (b) of this paragraph.

In particular, it was observed that in plants, especially in Arabidopsis thaliana, increasing or generating the activity of a gene product plastidic with the activity of a “glutamate dehydrogenase”, preferably being encoded by a gene comprising the nucleic acid sequence SEQ ID NO. 136943, preferably the coding region thereof, conferred the production of or the increase in succinate compared with the wild type control.

The nucleic acid sequence of Ybl015w from Saccharomyces cerevisiae, e.g. as shown in the respective line in column 5 of Table I, application no. 15, has been published in A. And the activity of the gene product thereof is the activity of acetyl-CoA hydrolase.

Accordingly, in one embodiment, the process of the present invention for producing citramalate in a non-human organism, like a microorganism or a plant or a part thereof, comprises increasing or generating the activity of a gene product with the activity of a gene product conferring the activity of “acetyl-CoA hydrolase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said Ybl015w, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Ybl015w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Ybl015w, or a functional equivalent or a homolog thereof as depicted in column 8 of Table II, application no. 15, preferably a homolog or functional equivalent as depicted in column 8 of Table II B, application no. 15, and being depicted in the same respective line as said Ybl015w, and preferably the activity is increased non-targeted, whereby the respective line disclose in column 7 the fine chemical citramalate.

Accordingly, in one embodiment, the molecule which activity is to be increased in the process of the invention is the gene product with an activity as a “acetyl-CoA hydrolase”, preferably it is the molecule of section (a) or (b) of this paragraph.

In particular, it was observed that in plants, especially in Arabidopsis thaliana, increasing or generating the activity of a gene product non-targeted with the activity of a “acetyl-CoA hydrolase”, preferably being encoded by a gene comprising the nucleic acid sequence SEQ ID NO. 137387, preferably the coding region thereof, conferred the production of or the increase in citramalate compared with the wild type control.

The nucleic acid sequence of Ybl015w from Saccharomyces cerevisiae, e.g. as shown in the respective line in column 5 of Table I, application no. 15, has been published in A. And the activity of the gene product thereof is the activity of acetyl-CoA hydrolase.

Accordingly, in one embodiment, the process of the present invention for producing pyruvate in a non-human organism, like a microorganism or a plant or a part thereof, comprises increasing or generating the activity of a gene product with the activity of a gene product conferring the activity of “acetyl-CoA hydrolase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical pyruvate), application no. 15, preferably the coding region thereof, or a homolog or a fragment thereof, and being depicted in the same respective line as said Ybl015w, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Ybl015w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Ybl015w, or a functional equivalent or a homolog thereof as depicted in column 8 of Table II, application no. 15, preferably a homolog or functional equivalent as depicted in column 8 of Table II B, application no. 15, and being depicted in the same respective line as said Ybl015w, and preferably the activity is increased non-targeted, whereby the respective line disclose in column 7 the fine chemical pyruvate.

Accordingly, in one embodiment, the molecule which activity is to be increased in the process of the invention is the gene product with an activity as a “acetyl-CoA hydrolase”, preferably it is the molecule of section (a) or (b) of this paragraph.

In particular, it was observed that in plants, especially in Arabidopsis thaliana, increasing or generating the activity of a gene product non-targeted with the activity of a “acetyl-CoA hydrolase”, preferably being encoded by a gene comprising the nucleic acid sequence SEQ ID NO. 137387, preferably the coding region thereof, conferred the production of or the increase in pyruvate compared with the wild type control.

The nucleic acid sequence of Ybr218c from Saccharomyces cerevisiae, e.g. as shown in the respective line in column 5 of Table I, application no. 15, has been published in A. And the activity of the gene product thereof is the activity of pyruvate carboxylase.

Accordingly, in one embodiment, the process of the present invention for producing malate in a non-human organism, like a microorganism or a plant or a part thereof, comprises increasing or generating the activity of a gene product with the activity of a gene product conferring the activity of “pyruvate carboxylase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said Ybr218c, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Ybr218c, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Ybr218c, or a functional equivalent or a homolog thereof as depicted in column 8 of Table II, application no. 15, preferably a homolog or functional equivalent as depicted in column 8 of Table II B, application no. 15, and being depicted in the same respective line as said Ybr218c, and preferably the activity is increased plastidic, whereby the respective line disclose in column 7 the fine chemical malate.

Accordingly, in one embodiment, the molecule which activity is to be increased in the process of the invention is the gene product with an activity as a “pyruvate carboxylase”, preferably it is the molecule of section (a) or (b) of this paragraph.

In particular, it was observed that in plants, especially in Arabidopsis thaliana, increasing or generating the activity of a gene product plastidic with the activity of a “pyruvate carboxylase”, preferably being encoded by a gene comprising the nucleic acid sequence SEQ ID NO. 96779, preferably the coding region thereof, conferred the production of or the increase in malate compared with the wild type control.

The nucleic acid sequence of Ycr102c from Saccharomyces cerevisiae, e.g. as shown in the respective line in column 5 of Table I, application no. 15, has been published in A. And the activity of the gene product thereof is the activity of ycr102c-protein.

Accordingly, in one embodiment, the process of the present invention for producing fumarate in a non-human organism, like a microorganism or a plant or a part thereof, comprises increasing or generating the activity of a gene product with the activity of a gene product conferring the activity of “ycr102c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said Ycr102c, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Ycr102c, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Ycr102c, or a functional equivalent or a homolog thereof as depicted in column 8 of Table II, application no. 15, preferably a homolog or functional equivalent as depicted in column 8 of Table II B, application no. 15, and being depicted in the same respective line as said Ycr102c, and preferably the activity is increased plastidic, whereby the respective line disclose in column 7 the fine chemical fumarate.

Accordingly, in one embodiment, the molecule which activity is to be increased in the process of the invention is the gene product with an activity as a “ycr102c-protein”, preferably it is the molecule of section (a) or (b) of this paragraph.

In particular, it was observed that in plants, especially in Arabidopsis thaliana, increasing or generating the activity of a gene product plastidic with the activity of a “ycr102c-protein”, preferably being encoded by a gene comprising the nucleic acid sequence SEQ ID NO. 63264, preferably the coding region thereof, conferred the production of or the increase in fumarate compared with the wild type control.

The nucleic acid sequence of Ydl159w-a from Saccharomyces cerevisiae, e.g. as shown in the respective line in column 5 of Table I, application no. 15, has been published in A. And the activity of the gene product thereof is the activity of YDL159W-A-protein.

Accordingly, in one embodiment, the process of the present invention for producing fumarate in a non-human organism, like a microorganism or a plant or a part thereof, comprises increasing or generating the activity of a gene product with the activity of a gene product conferring the activity of “YDL159W-A-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said Ydl159w-a, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Ydl159w-a, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Ydl159w-a, or a functional equivalent or a homolog thereof as depicted in column 8 of Table II, application no. 15, preferably a homolog or functional equivalent as depicted in column 8 of Table II B, application no. 15, and being depicted in the same respective line as said Yd1159w-a, and preferably the activity is increased non-targeted, whereby the respective line disclose in column 7 the fine chemical fumarate.

Accordingly, in one embodiment, the molecule which activity is to be increased in the process of the invention is the gene product with an activity as a “YDL159W-A-protein”, preferably it is the molecule of section (a) or (b) of this paragraph.

In particular, it was observed that in plants, especially in Arabidopsis thaliana, increasing or generating the activity of a gene product non-targeted with the activity of a “YDL159W-A-protein”, preferably being encoded by a gene comprising the nucleic acid sequence SEQ ID NO. 137498, preferably the coding region thereof, conferred the production of or the increase in fumarate compared with the wild type control.

The nucleic acid sequence of Ydr044w from Saccharomyces cerevisiae, e.g. as shown in the respective line in column 5 of Table I, application no. 15, has been published in A. And the activity of the gene product thereof is the activity of coproporphyrinogen III oxidase.

Accordingly, in one embodiment, the process of the present invention for producing fumarate in a non-human organism, like a microorganism or a plant or a part thereof, comprises increasing or generating the activity of a gene product with the activity of a gene product conferring the activity of “coproporphyrinogen III oxidase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said Ydr044w, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Ydr044w, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Ydr044w, or a functional equivalent or a homolog thereof as depicted in column 8 of Table II, application no. 15, preferably a homolog or functional equivalent as depicted in column 8 of Table II B, application no. 15, and being depicted in the same respective line as said Ydr044w, and preferably the activity is increased plastidic, whereby the respective line disclose in column 7 the fine chemical fumarate.

Accordingly, in one embodiment, the molecule which activity is to be increased in the process of the invention is the gene product with an activity as a “coproporphyrinogen III oxidase”, preferably it is the molecule of section (a) or (b) of this paragraph.

In particular, it was observed that in plants, especially in Arabidopsis thaliana, increasing or generating the activity of a gene product plastidic with the activity of a “coproporphyrinogen III oxidase”, preferably being encoded by a gene comprising the nucleic acid sequence SEQ ID NO. 63334, preferably the coding region thereof, conferred the production of or the increase in fumarate compared with the wild type control.

The nucleic acid sequence of Ydr044w from Saccharomyces cerevisiae, e.g. as shown in the respective line in column 5 of Table I, application no. 15, has been published in A. And the activity of the gene product thereof is the activity of coproporphyrinogen III oxidase.

Accordingly, in one embodiment, the process of the present invention for producing succinate in a non-human organism, like a microorganism or a plant or a part thereof, comprises increasing or generating the activity of a gene product with the activity of a gene product conferring the activity of “coproporphyrinogen III oxidase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said Ydr044w, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Ydr044w, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Ydr044w, or a functional equivalent or a homolog thereof as depicted in column 8 of Table II, application no. 15, preferably a homolog or functional equivalent as depicted in column 8 of Table II B, application no. 15, and being depicted in the same respective line as said Ydr044w, and preferably the activity is increased plastidic, whereby the respective line disclose in column 7 the fine chemical succinate.

Accordingly, in one embodiment, the molecule which activity is to be increased in the process of the invention is the gene product with an activity as a “coproporphyrinogen III oxidase”, preferably it is the molecule of section (a) or (b) of this paragraph.

In particular, it was observed that in plants, especially in Arabidopsis thaliana, increasing or generating the activity of a gene product plastidic with the activity of a “coproporphyrinogen III oxidase”, preferably being encoded by a gene comprising the nucleic acid sequence SEQ ID NO. 63334, preferably the coding region thereof, conferred the production of or the increase in succinate compared with the wild type control.

The nucleic acid sequence of Ygl237c from Saccharomyces cerevisiae, e.g. as shown in the respective line in column 5 of Table I, application no. 15, has been published in A. And the activity of the gene product thereof is the activity of ygl237c-protein.

Accordingly, in one embodiment, the process of the present invention for producing malate in a non-human organism, like a microorganism or a plant or a part thereof, comprises increasing or generating the activity of a gene product with the activity of a gene product conferring the activity of “ygl237c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said Ygl237c, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Ygl237c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Ygl237c, or a functional equivalent or a homolog thereof as depicted in column 8 of Table II, application no. 15, preferably a homolog or functional equivalent as depicted in column 8 of Table II B, application no. 15, and being depicted in the same respective line as said Ygl237c, and preferably the activity is increased non-targeted, whereby the respective line disclose in column 7 the fine chemical malate.

Accordingly, in one embodiment, the molecule which activity is to be increased in the process of the invention is the gene product with an activity as a “ygl237c-protein”, preferably it is the molecule of section (a) or (b) of this paragraph.

In particular, it was observed that in plants, especially in Arabidopsis thaliana, increasing or generating the activity of a gene product non-targeted with the activity of a “ygl237c-protein”, preferably being encoded by a gene comprising the nucleic acid sequence SEQ ID NO. 64177, preferably the coding region thereof, conferred the production of or the increase in malate compared with the wild type control.

The nucleic acid sequence of Ygl237c from Saccharomyces cerevisiae, e.g. as shown in the respective line in column 5 of Table I, application no. 15, has been published in A. And the activity of the gene product thereof is the activity of ygl237c-protein.

Accordingly, in one embodiment, the process of the present invention for producing succinate in a non-human organism, like a microorganism or a plant or a part thereof, comprises increasing or generating the activity of a gene product with the activity of a gene product conferring the activity of “ygl237c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said Ygl237c, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Ygl237c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Ygl237c, or a functional equivalent or a homolog thereof as depicted in column 8 of Table II, application no. 15, preferably a homolog or functional equivalent as depicted in column 8 of Table II B, application no. 15, and being depicted in the same respective line as said Ygl237c, and preferably the activity is increased non-targeted, whereby the respective line disclose in column 7 the fine chemical succinate.

Accordingly, in one embodiment, the molecule which activity is to be increased in the process of the invention is the gene product with an activity as a “ygl237c-protein”, preferably it is the molecule of section (a) or (b) of this paragraph.

In particular, it was observed that in plants, especially in Arabidopsis thaliana, increasing or generating the activity of a gene product non-targeted with the activity of a “ygl237c-protein”, preferably being encoded by a gene comprising the nucleic acid sequence SEQ ID NO. 64177, preferably the coding region thereof, conferred the production of or the increase in succinate compared with the wild type control.

The nucleic acid sequence of Ygr193c from Saccharomyces cerevisiae, e.g. as shown in the respective line in column 5 of Table I, application no. 15, has been published in A. And the activity of the gene product thereof is the activity of YGR193C-protein.

Accordingly, in one embodiment, the process of the present invention for producing fumarate in a non-human organism, like a microorganism or a plant or a part thereof, comprises increasing or generating the activity of a gene product with the activity of a gene product conferring the activity of “YGR193C-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said Ygr193c, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Ygr193c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Ygr193c, or a functional equivalent or a homolog thereof as depicted in column 8 of Table II, application no. 15, preferably a homolog or functional equivalent as depicted in column 8 of Table II B, application no. 15, and being depicted in the same respective line as said Ygr193c, and preferably the activity is increased non-targeted, whereby the respective line disclose in column 7 the fine chemical fumarate.

Accordingly, in one embodiment, the molecule which activity is to be increased in the process of the invention is the gene product with an activity as a “YGR193C-protein”, preferably it is the molecule of section (a) or (b) of this paragraph.

In particular, it was observed that in plants, especially in Arabidopsis thaliana, increasing or generating the activity of a gene product non-targeted with the activity of a “YGR193C-protein”, preferably being encoded by a gene comprising the nucleic acid sequence SEQ ID NO. 108487, preferably the coding region thereof, conferred the production of or the increase in fumarate compared with the wild type control.

The nucleic acid sequence of Ygr193c from Saccharomyces cerevisiae, e.g. as shown in the respective line in column 5 of Table I, application no. 15, has been published in A. And the activity of the gene product thereof is the activity of YGR193C-protein.

Accordingly, in one embodiment, the process of the present invention for producing malate in a non-human organism, like a microorganism or a plant or a part thereof, comprises increasing or generating the activity of a gene product with the activity of a gene product conferring the activity of “YGR193C-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said Ygr193c, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Ygr193c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Ygr193c, or a functional equivalent or a homolog thereof as depicted in column 8 of Table II, application no. 15, preferably a homolog or functional equivalent as depicted in column 8 of Table II B, application no. 15, and being depicted in the same respective line as said Ygr193c, and preferably the activity is increased non-targeted, whereby the respective line disclose in column 7 the fine chemical malate.

Accordingly, in one embodiment, the molecule which activity is to be increased in the process of the invention is the gene product with an activity as a “YGR193C-protein”, preferably it is the molecule of section (a) or (b) of this paragraph.

In particular, it was observed that in plants, especially in Arabidopsis thaliana, increasing or generating the activity of a gene product non-targeted with the activity of a “YGR193C-protein”, preferably being encoded by a gene comprising the nucleic acid sequence SEQ ID NO. 108487, preferably the coding region thereof, conferred the production of or the increase in malate compared with the wild type control.

The nucleic acid sequence of Ygr193c from Saccharomyces cerevisiae, e.g. as shown in the respective line in column 5 of Table I, application no. 15, has been published in A. And the activity of the gene product thereof is the activity of YGR193C-protein.

Accordingly, in one embodiment, the process of the present invention for producing succinate in a non-human organism, like a microorganism or a plant or a part thereof, comprises increasing or generating the activity of a gene product with the activity of a gene product conferring the activity of “YGR193C-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said Ygr193c, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Ygr193c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Ygr193c, or a functional equivalent or a homolog thereof as depicted in column 8 of Table II, application no. 15, preferably a homolog or functional equivalent as depicted in column 8 of Table II B, application no. 15, and being depicted in the same respective line as said Ygr193c, and preferably the activity is increased non-targeted, whereby the respective line disclose in column 7 the fine chemical succinate.

Accordingly, in one embodiment, the molecule which activity is to be increased in the process of the invention is the gene product with an activity as a “YGR193C-protein”, preferably it is the molecule of section (a) or (b) of this paragraph.

In particular, it was observed that in plants, especially in Arabidopsis thaliana, increasing or generating the activity of a gene product non-targeted with the activity of a “YGR193C-protein”, preferably being encoded by a gene comprising the nucleic acid sequence SEQ ID NO. 108487, preferably the coding region thereof, conferred the production of or the increase in succinate compared with the wild type control.

The nucleic acid sequence of Ygr221 c from Saccharomyces cerevisiae, e.g. as shown in the respective line in column 5 of Table I, application no. 15, has been published in A. And the activity of the gene product thereof is the activity of ygr221c-protein.

Accordingly, in one embodiment, the process of the present invention for producing malate in a non-human organism, like a microorganism or a plant or a part thereof, comprises increasing or generating the activity of a gene product with the activity of a gene product conferring the activity of “ygr221 c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said Ygr221 c, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Ygr221c, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Ygr221c, or a functional equivalent or a homolog thereof as depicted in column 8 of Table II, application no. 15, preferably a homolog or functional equivalent as depicted in column 8 of Table II B, application no. 15, and being depicted in the same respective line as said Ygr221 c, and preferably the activity is increased plastidic, whereby the respective line disclose in column 7 the fine chemical malate.

Accordingly, in one embodiment, the molecule which activity is to be increased in the process of the invention is the gene product with an activity as a “ygr221c-protein”, preferably it is the molecule of section (a) or (b) of this paragraph.

In particular, it was observed that in plants, especially in Arabidopsis thaliana, increasing or generating the activity of a gene product plastidic with the activity of a “ygr221 c-protein”, preferably being encoded by a gene comprising the nucleic acid sequence SEQ ID NO. 64315, preferably the coding region thereof, conferred the production of or the increase in malate compared with the wild type control.

The nucleic acid sequence of Yhl032c from Saccharomyces cerevisiae, e.g. as shown in the respective line in column 5 of Table I, application no. 15, has been published in A. And the activity of the gene product thereof is the activity of glycerol kinase.

Accordingly, in one embodiment, the process of the present invention for producing citrate in a non-human organism, like a microorganism or a plant or a part thereof, comprises increasing or generating the activity of a gene product with the activity of a gene product conferring the activity of “glycerol kinase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical citrate), application no. 15, preferably the coding region thereof, or a homolog or a fragment thereof, and being depicted in the same respective line as said Yhl032c, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Yhl032c, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Yhl032c, or a functional equivalent or a homolog thereof as depicted in column 8 of Table II, application no. 15, preferably a homolog or functional equivalent as depicted in column 8 of Table II B, application no. 15, and being depicted in the same respective line as said Yhl032c, and preferably the activity is increased plastidic, whereby the respective line disclose in column 7 the fine chemical citrate.

Accordingly, in one embodiment, the molecule which activity is to be increased in the process of the invention is the gene product with an activity as a “glycerol kinase”, preferably it is the molecule of section (a) or (b) of this paragraph.

In particular, it was observed that in plants, especially in Arabidopsis thaliana, increasing or generating the activity of a gene product plastidic with the activity of a “glycerol kinase”, preferably being encoded by a gene comprising the nucleic acid sequence SEQ ID NO. 101855, preferably the coding region thereof, conferred the production of or the increase in citrate compared with the wild type control.

The nucleic acid sequence of Yhr131c from Saccharomyces cerevisiae, e.g. as shown in the respective line in column 5 of Table I, application no. 15, has been published in A. And the activity of the gene product thereof is the activity of yhr131c-protein.

Accordingly, in one embodiment, the process of the present invention for producing malate in a non-human organism, like a microorganism or a plant or a part thereof, comprises increasing or generating the activity of a gene product with the activity of a gene product conferring the activity of “yhr131c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said Yhr131c, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Yhr131c, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Yhr131c, or a functional equivalent or a homolog thereof as depicted in column 8 of Table II, application no. 15, preferably a homolog or functional equivalent as depicted in column 8 of Table II B, application no. 15, and being depicted in the same respective line as said Yhr131c, and preferably the activity is increased plastidic, whereby the respective line disclose in column 7 the fine chemical malate.

Accordingly, in one embodiment, the molecule which activity is to be increased in the process of the invention is the gene product with an activity as a “yhr131c-protein”, preferably it is the molecule of section (a) or (b) of this paragraph.

In particular, it was observed that in plants, especially in Arabidopsis thaliana, increasing or generating the activity of a gene product plastidic with the activity of a “yhr131c-protein”, preferably being encoded by a gene comprising the nucleic acid sequence SEQ ID NO. 97620, preferably the coding region thereof, conferred the production of or the increase in malate compared with the wild type control.

The nucleic acid sequence of Yhr207c from Saccharomyces cerevisiae, e.g. as shown in the respective line in column 5 of Table I, application no. 15, has been published in A. And the activity of the gene product thereof is the activity of yhr207c-protein.

Accordingly, in one embodiment, the process of the present invention for producing succinate in a non-human organism, like a microorganism or a plant or a part thereof, comprises increasing or generating the activity of a gene product with the activity of a gene product conferring the activity of “yhr207c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said Yhr207c, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Yhr207c, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Yhr207c, or a functional equivalent or a homolog thereof as depicted in column 8 of Table II, application no. 15, preferably a homolog or functional equivalent as depicted in column 8 of Table II B, application no. 15, and being depicted in the same respective line as said

Yhr207c, and preferably the activity is increased plastidic, whereby the respective line disclose in column 7 the fine chemical succinate.

Accordingly, in one embodiment, the molecule which activity is to be increased in the process of the invention is the gene product with an activity as a “yhr207c-protein”, preferably it is the molecule of section (a) or (b) of this paragraph.

In particular, it was observed that in plants, especially in Arabidopsis thaliana, increasing or generating the activity of a gene product plastidic with the activity of a “yhr207c-protein”, preferably being encoded by a gene comprising the nucleic acid sequence SEQ ID NO. 64546, preferably the coding region thereof, conferred the production of or the increase in succinate compared with the wild type control.

The nucleic acid sequence of Yjl073w from Saccharomyces cerevisiae, e.g. as shown in the respective line in column 5 of Table I, application no. 15, has been published in A. And the activity of the gene product thereof is the activity of DnaJ-like chaperone.

Accordingly, in one embodiment, the process of the present invention for producing glycerate in a non-human organism, like a microorganism or a plant or a part thereof, comprises increasing or generating the activity of a gene product with the activity of a gene product conferring the activity of “DnaJ-like chaperone”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glycerate), application no. 15, preferably the coding region thereof, or a homolog or a fragment thereof, and being depicted in the same respective line as said Yjl073w, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Yjl073w, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Yjl073w, or a functional equivalent or a homolog thereof as depicted in column 8 of Table II, application no. 15, preferably a homolog or functional equivalent as depicted in column 8 of Table II B, application no. 15, and being depicted in the same respective line as said Yjl073w, and preferably the activity is increased plastidic, whereby the respective line disclose in column 7 the fine chemical glycerate.

Accordingly, in one embodiment, the molecule which activity is to be increased in the process of the invention is the gene product with an activity as a “DnaJ-like chaperone”, preferably it is the molecule of section (a) or (b) of this paragraph.

In particular, it was observed that in plants, especially in Arabidopsis thaliana, increasing or generating the activity of a gene product plastidic with the activity of a “DnaJ-like chaperone”, preferably being encoded by a gene comprising the nucleic acid sequence SEQ ID NO. 64964, preferably the coding region thereof, conferred the production of or the increase in glycerate compared with the wild type control.

The nucleic acid sequence of Yjl219w from Saccharomyces cerevisiae, e.g. as shown in the respective line in column 5 of Table I, application no. 15, has been published in A. And the activity of the gene product thereof is the activity of hexose transporter.

Accordingly, in one embodiment, the process of the present invention for producing oxalate in a non-human organism, like a microorganism or a plant or a part thereof, comprises increasing or generating the activity of a gene product with the activity of a gene product conferring the activity of “hexose transporter”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical oxalate), application no. 15, preferably the coding region thereof, or a homolog or a fragment thereof, and being depicted in the same respective line as said Yjl219w, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Yjl219w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Yjl219w, or a functional equivalent or a homolog thereof as depicted in column 8 of Table II, application no. 15, preferably a homolog or functional equivalent as depicted in column 8 of Table II B, application no. 15, and being depicted in the same respective line as said Yjl219w, and preferably the activity is increased non-targeted, whereby the respective line disclose in column 7 the fine chemical oxalate.

Accordingly, in one embodiment, the molecule which activity is to be increased in the process of the invention is the gene product with an activity as a “hexose transporter”, preferably it is the molecule of section (a) or (b) of this paragraph.

In particular, it was observed that in plants, especially in Arabidopsis thaliana, increasing or generating the activity of a gene product non-targeted with the activity of a “hexose transporter”, preferably being encoded by a gene comprising the nucleic acid sequence SEQ ID NO. 97635, preferably the coding region thereof, conferred the production of or the increase in oxalate compared with the wild type control.

The nucleic acid sequence of Yjr148w from Saccharomyces cerevisiae, e.g. as shown in the respective line in column 5 of Table I, application no. 15, has been published in A. And the activity of the gene product thereof is the activity of branched chain aminotransferase.

Accordingly, in one embodiment, the process of the present invention for producing citramalate in a non-human organism, like a microorganism or a plant or a part thereof, comprises increasing or generating the activity of a gene product with the activity of a gene product conferring the activity of “branched chain aminotransferase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said Yjr148w, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Yjr148w, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Yjr148w, or a functional equivalent or a homolog thereof as depicted in column 8 of Table II, application no. 15, preferably a homolog or functional equivalent as depicted in column 8 of Table II B, application no. 15, and being depicted in the same respective line as said Yjr148w, and preferably the activity is increased plastidic, whereby the respective line disclose in column 7 the fine chemical citramalate.

Accordingly, in one embodiment, the molecule which activity is to be increased in the process of the invention is the gene product with an activity as a “branched chain aminotransferase”, preferably it is the molecule of section (a) or (b) of this paragraph.

In particular, it was observed that in plants, especially in Arabidopsis thaliana, increasing or generating the activity of a gene product plastidic with the activity of a “branched chain aminotransferase”, preferably being encoded by a gene comprising the nucleic acid sequence SEQ ID NO. 90409, preferably the coding region thereof, conferred the production of or the increase in citramalate compared with the wild type control.

The nucleic acid sequence of Yjr153w from Saccharomyces cerevisiae, e.g. as shown in the respective line in column 5 of Table I, application no. 15, has been published in A. And the activity of the gene product thereof is the activity of polygalacturonase.

Accordingly, in one embodiment, the process of the present invention for producing fumarate in a non-human organism, like a microorganism or a plant or a part thereof, comprises increasing or generating the activity of a gene product with the activity of a gene product conferring the activity of “polygalacturonase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said Yjr153w, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Yjr153w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Yjr153w, or a functional equivalent or a homolog thereof as depicted in column 8 of Table II, application no. 15, preferably a homolog or functional equivalent as depicted in column 8 of Table II B, application no. 15, and being depicted in the same respective line as said Yjr153w, and preferably the activity is increased non-targeted, whereby the respective line disclose in column 7 the fine chemical fumarate.

Accordingly, in one embodiment, the molecule which activity is to be increased in the process of the invention is the gene product with an activity as a “polygalacturonase”, preferably it is the molecule of section (a) or (b) of this paragraph.

In particular, it was observed that in plants, especially in Arabidopsis thaliana, increasing or generating the activity of a gene product non-targeted with the activity of a “polygalacturonase”, preferably being encoded by a gene comprising the nucleic acid sequence SEQ ID NO. 66274, preferably the coding region thereof, conferred the production of or the increase in fumarate compared with the wild type control.

The nucleic acid sequence of Yjr153w from Saccharomyces cerevisiae, e.g. as shown in the respective line in column 5 of Table I, application no. 15, has been published in A. And the activity of the gene product thereof is the activity of polygalacturonase.

Accordingly, in one embodiment, the process of the present invention for producing malate in a non-human organism, like a microorganism or a plant or a part thereof, comprises increasing or generating the activity of a gene product with the activity of a gene product conferring the activity of “polygalacturonase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said Yjr153w, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Yjr153w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Yjr153w, or a functional equivalent or a homolog thereof as depicted in column 8 of Table II, application no. 15, preferably a homolog or functional equivalent as depicted in column 8 of Table II B, application no. 15, and being depicted in the same respective line as said Yjr153w, and preferably the activity is increased non-targeted, whereby the respective line disclose in column 7 the fine chemical malate.

Accordingly, in one embodiment, the molecule which activity is to be increased in the process of the invention is the gene product with an activity as a “polygalacturonase”, preferably it is the molecule of section (a) or (b) of this paragraph.

In particular, it was observed that in plants, especially in Arabidopsis thaliana, increasing or generating the activity of a gene product non-targeted with the activity of a “polygalacturonase”, preferably being encoded by a gene comprising the nucleic acid sequence SEQ ID NO. 66274, preferably the coding region thereof, conferred the production of or the increase in malate compared with the wild type control.

The nucleic acid sequence of YKL038W from Saccharomyces cerevisiae, e.g. as shown in the respective line in column 5 of Table I, application no. 15, has been published in A. And the activity of the gene product thereof is the activity of YKL038W-protein.

Accordingly, in one embodiment, the process of the present invention for producing malate in a non-human organism, like a microorganism or a plant or a part thereof, comprises increasing or generating the activity of a gene product with the activity of a gene product conferring the activity of “YKL038W-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said YKL038W, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said YKL038W, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said YKL038W, or a functional equivalent or a homolog thereof as depicted in column 8 of Table II, application no. 15, preferably a homolog or functional equivalent as depicted in column 8 of Table II B, application no. 15, and being depicted in the same respective line as said YKL038W, and preferably the activity is increased plastidic, whereby the respective line disclose in column 7 the fine chemical malate.

Accordingly, in one embodiment, the molecule which activity is to be increased in the process of the invention is the gene product with an activity as a “YKL038W-protein”, preferably it is the molecule of section (a) or (b) of this paragraph.

In particular, it was observed that in plants, especially in Arabidopsis thaliana, increasing or generating the activity of a gene product plastidic with the activity of a “YKL038W-protein”, preferably being encoded by a gene comprising the nucleic acid sequence SEQ ID NO. 90602, preferably the coding region thereof, conferred the production of or the increase in malate compared with the wild type control.

The nucleic acid sequence of Ykl140w from Saccharomyces cerevisiae, e.g. as shown in the respective line in column 5 of Table I, application no. 15, has been published in A. And the activity of the gene product thereof is the activity of YKL140W-protein.

Accordingly, in one embodiment, the process of the present invention for producing vitamin c and/or threonic acid in a non-human organism, like a microorganism or a plant or a part thereof, comprises increasing or generating the activity of a gene product with the activity of a gene product conferring the activity of “YKL140W-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical vitamin c and/or threonic acid), application no. 15, preferably the coding region thereof, or a homolog or a fragment thereof, and being depicted in the same respective line as said Ykl140w, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Ykl140w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Ykl140w, or a functional equivalent or a homolog thereof as depicted in column 8 of Table II, application no. 15, preferably a homolog or functional equivalent as depicted in column 8 of Table II B, application no. 15, and being depicted in the same respective line as said Ykl140w, and preferably the activity is increased non-targeted, whereby the respective line disclose in column 7 the fine chemical vitamin c and/or threonic acid.

Accordingly, in one embodiment, the molecule which activity is to be increased in the process of the invention is the gene product with an activity as a “YKL140W-protein”, preferably it is the molecule of section (a) or (b) of this paragraph.

In particular, it was observed that in plants, especially in Arabidopsis thaliana, increasing or generating the activity of a gene product non-targeted with the activity of a “YKL140W-protein”, preferably being encoded by a gene comprising the nucleic acid sequence SEQ ID NO. 137502, preferably the coding region thereof, conferred the production of or the increase in vitamin c and/or threonic acid compared with the wild type control.

The nucleic acid sequence of Ylr065c from Saccharomyces cerevisiae, e.g. as shown in the respective line in column 5 of Table I, application no. 15, has been published in A. And the activity of the gene product thereof is the activity of ylr065c-protein.

Accordingly, in one embodiment, the process of the present invention for producing fumarate in a non-human organism, like a microorganism or a plant or a part thereof, comprises increasing or generating the activity of a gene product with the activity of a gene product conferring the activity of “ylr065c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said Ylr065c, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Ylr065c, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Ylr065c, or a functional equivalent or a homolog thereof as depicted in column 8 of Table II, application no. 15, preferably a homolog or functional equivalent as depicted in column 8 of Table II B, application no. 15, and being depicted in the same respective line as said Ylr065c, and preferably the activity is increased plastidic, whereby the respective line disclose in column 7 the fine chemical fumarate.

Accordingly, in one embodiment, the molecule which activity is to be increased in the process of the invention is the gene product with an activity as a “ylr065c-protein”, preferably it is the molecule of section (a) or (b) of this paragraph.

In particular, it was observed that in plants, especially in Arabidopsis thaliana, increasing or generating the activity of a gene product plastidic with the activity of a “ylr065c-protein”, preferably being encoded by a gene comprising the nucleic acid sequence SEQ ID NO. 66695, preferably the coding region thereof, conferred the production of or the increase in fumarate compared with the wild type control.

The nucleic acid sequence of Ylr174w from Saccharomyces cerevisiae, e.g. as shown in the respective line in column 5 of Table I, application no. 15, has been published in A. And the activity of the gene product thereof is the activity of isocitrate dehydrogenase.

Accordingly, in one embodiment, the process of the present invention for producing succinate in a non-human organism, like a microorganism or a plant or a part thereof, comprises increasing or generating the activity of a gene product with the activity of a gene product conferring the activity of “isocitrate dehydrogenase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said Ylr174w, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Ylr174w, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Ylr174w, or a functional equivalent or a homolog thereof as depicted in column 8 of Table II, application no. 15, preferably a homolog or functional equivalent as depicted in column 8 of Table II B, application no. 15, and being depicted in the same respective line as said Ylr174w, and preferably the activity is increased plastidic, whereby the respective line disclose in column 7 the fine chemical succinate.

Accordingly, in one embodiment, the molecule which activity is to be increased in the process of the invention is the gene product with an activity as a “isocitrate dehydrogenase”, preferably it is the molecule of section (a) or (b) of this paragraph.

In particular, it was observed that in plants, especially in Arabidopsis thaliana, increasing or generating the activity of a gene product plastidic with the activity of a “isocitrate dehydrogenase”, preferably being encoded by a gene comprising the nucleic acid sequence SEQ ID NO. 137547, preferably the coding region thereof, conferred the production of or the increase in succinate compared with the wild type control.

The nucleic acid sequence of Ylr326w from Saccharomyces cerevisiae, e.g. as shown in the respective line in column 5 of Table I, application no. 15, has been published in A. And the activity of the gene product thereof is the activity of ylr326w-protein.

Accordingly, in one embodiment, the process of the present invention for producing fumarate in a non-human organism, like a microorganism or a plant or a part thereof, comprises increasing or generating the activity of a gene product with the activity of a gene product conferring the activity of “ylr326w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said Ylr326w, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Ylr326w, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Ylr326w, or a functional equivalent or a homolog thereof as depicted in column 8 of Table II, application no. 15, preferably a homolog or functional equivalent as depicted in column 8 of Table II B, application no. 15, and being depicted in the same respective line as said Ylr326w, and preferably the activity is increased plastidic, whereby the respective line disclose in column 7 the fine chemical fumarate.

Accordingly, in one embodiment, the molecule which activity is to be increased in the process of the invention is the gene product with an activity as a “ylr326w-protein”, preferably it is the molecule of section (a) or (b) of this paragraph.

In particular, it was observed that in plants, especially in Arabidopsis thaliana, increasing or generating the activity of a gene product plastidic with the activity of a “ylr326w-protein”, preferably being encoded by a gene comprising the nucleic acid sequence SEQ ID NO. 129123, preferably the coding region thereof, conferred the production of or the increase in fumarate compared with the wild type control.

The nucleic acid sequence of Ylr326w from Saccharomyces cerevisiae, e.g. as shown in the respective line in column 5 of Table I, application no. 15, has been published in A. And the activity of the gene product thereof is the activity of ylr326w-protein.

Accordingly, in one embodiment, the process of the present invention for producing glycerate in a non-human organism, like a microorganism or a plant or a part thereof, comprises increasing or generating the activity of a gene product with the activity of a gene product conferring the activity of “ylr326w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glycerate), application no. 15, preferably the coding region thereof, or a homolog or a fragment thereof, and being depicted in the same respective line as said Ylr326w, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Ylr326w, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Ylr326w, or a functional equivalent or a homolog thereof as depicted in column 8 of Table II, application no. 15, preferably a homolog or functional equivalent as depicted in column 8 of Table II B, application no. 15, and being depicted in the same respective line as said Ylr326w, and preferably the activity is increased plastidic, whereby the respective line disclose in column 7 the fine chemical glycerate.

Accordingly, in one embodiment, the molecule which activity is to be increased in the process of the invention is the gene product with an activity as a “ylr326w-protein”, preferably it is the molecule of section (a) or (b) of this paragraph.

In particular, it was observed that in plants, especially in Arabidopsis thaliana, increasing or generating the activity of a gene product plastidic with the activity of a “ylr326w-protein”, preferably being encoded by a gene comprising the nucleic acid sequence SEQ ID NO. 129123, preferably the coding region thereof, conferred the production of or the increase in glycerate compared with the wild type control.

The nucleic acid sequence of Ylr326w from Saccharomyces cerevisiae, e.g. as shown in the respective line in column 5 of Table I, application no. 15, has been published in A. And the activity of the gene product thereof is the activity of ylr326w-protein.

Accordingly, in one embodiment, the process of the present invention for producing vitamin c and/or threonic acid in a non-human organism, like a microorganism or a plant or a part thereof, comprises increasing or generating the activity of a gene product with the activity of a gene product conferring the activity of “ylr326w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical vitamin c and/or threonic acid), application no. 15, preferably the coding region thereof, or a homolog or a fragment thereof, and being depicted in the same respective line as said Ylr326w, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Ylr326w, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Ylr326w, or a functional equivalent or a homolog thereof as depicted in column 8 of Table II, application no. 15, preferably a homolog or functional equivalent as depicted in column 8 of Table II B, application no. 15, and being depicted in the same respective line as said Ylr326w, and preferably the activity is increased plastidic, whereby the respective line disclose in column 7 the fine chemical vitamin c and/or threonic acid.

Accordingly, in one embodiment, the molecule which activity is to be increased in the process of the invention is the gene product with an activity as a “ylr326w-protein”, preferably it is the molecule of section (a) or (b) of this paragraph.

In particular, it was observed that in plants, especially in Arabidopsis thaliana, increasing or generating the activity of a gene product plastidic with the activity of a “ylr326w-protein”, preferably being encoded by a gene comprising the nucleic acid sequence SEQ ID NO. 129123, preferably the coding region thereof, conferred the production of or the increase in vitamin c and/or threonic acid compared with the wild type control.

The nucleic acid sequence of Yml084w from Saccharomyces cerevisiae, e.g. as shown in the respective line in column 5 of Table I, application no. 15, has been published in A. And the activity of the gene product thereof is the activity of yml084w-protein.

Accordingly, in one embodiment, the process of the present invention for producing fumarate in a non-human organism, like a microorganism or a plant or a part thereof, comprises increasing or generating the activity of a gene product with the activity of a gene product conferring the activity of “yml084w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said Yml084w, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Yml084w, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Yml084w, or a functional equivalent or a homolog thereof as depicted in column 8 of Table II, application no. 15, preferably a homolog or functional equivalent as depicted in column 8 of Table II B, application no. 15, and being depicted in the same respective line as said Yml084w, and preferably the activity is increased plastidic, whereby the respective line disclose in column 7 the fine chemical fumarate.

Accordingly, in one embodiment, the molecule which activity is to be increased in the process of the invention is the gene product with an activity as a “yml084w-protein”, preferably it is the molecule of section (a) or (b) of this paragraph.

In particular, it was observed that in plants, especially in Arabidopsis thaliana, increasing or generating the activity of a gene product plastidic with the activity of a “yml084w-protein”, preferably being encoded by a gene comprising the nucleic acid sequence SEQ ID NO. 15175, preferably the coding region thereof, conferred the production of or the increase in fumarate compared with the wild type control.

The nucleic acid sequence of Yml084w from Saccharomyces cerevisiae, e.g. as shown in the respective line in column 5 of Table I, application no. 15, has been published in A. And the activity of the gene product thereof is the activity of yml084w-protein.

Accordingly, in one embodiment, the process of the present invention for producing succinate in a non-human organism, like a microorganism or a plant or a part thereof, comprises increasing or generating the activity of a gene product with the activity of a gene product conferring the activity of “yml084w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said Yml084w, or a functional equivalent or a homolog thereof as shown in column 8 of Table 1, application no. 15, preferably the coding region thereof, particularly a homolog or functional equivalent as shown in column 8 of Table I B, application no. 15, and being depicted in the same respective line as said Yml084w, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Yml084w, or a functional equivalent or a homolog thereof as depicted in column 8 of Table II, application no. 15, preferably a homolog or functional equivalent as depicted in column 8 of Table II B, application no. 15, and being depicted in the same respective line as said Yml084w, and preferably the activity is increased plastidic, whereby the respective line disclose in column 7 the fine chemical succinate.

Accordingly, in one embodiment, the molecule which activity is to be increased in the process of the invention is the gene product with an activity as a “yml084w-protein”, preferably it is the molecule of section (a) or (b) of this paragraph.

In particular, it was observed that in plants, especially in Arabidopsis thaliana, increasing or generating the activity of a gene product plastidic with the activity of a “yml084w-protein”, preferably being encoded by a gene comprising the nucleic acid sequence SEQ ID NO. 15175, preferably the coding region thereof, conferred the production of or the increase in succinate compared with the wild type control.

The nucleic acid sequence of Yol073c from Saccharomyces cerevisiae, e.g. as shown in the respective line in column 5 of Table I, application no. 15, has been published in A. And the activity of the gene product thereof is the activity of yol073c-protein.

Accordingly, in one embodiment, the process of the present invention for producing citramalate in a non-human organism, like a microorganism or a plant or a part thereof, comprises increasing or generating the activity of a gene product with the activity of a gene product conferring the activity of “yol073c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said Yol073c, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Yol073c, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Yol073c, or a functional equivalent or a homolog thereof as depicted in column 8 of Table II, application no. 15, preferably a homolog or functional equivalent as depicted in column 8 of Table II B, application no. 15, and being depicted in the same respective line as said Yol073c, and preferably the activity is increased plastidic, whereby the respective line disclose in column 7 the fine chemical citramalate.

Accordingly, in one embodiment, the molecule which activity is to be increased in the process of the invention is the gene product with an activity as a “yol073c-protein”, preferably it is the molecule of section (a) or (b) of this paragraph.

In particular, it was observed that in plants, especially in Arabidopsis thaliana, increasing or generating the activity of a gene product plastidic with the activity of a “yol073c-protein”, preferably being encoded by a gene comprising the nucleic acid sequence SEQ ID NO. 137818, preferably the coding region thereof, conferred the production of or the increase in citramalate compared with the wild type control.

The nucleic acid sequence of Yol073c from Saccharomyces cerevisiae, e.g. as shown in the respective line in column 5 of Table I, application no. 15, has been published in A. And the activity of the gene product thereof is the activity of yol073c-protein.

Accordingly, in one embodiment, the process of the present invention for producing malate in a non-human organism, like a microorganism or a plant or a part thereof, comprises increasing or generating the activity of a gene product with the activity of a gene product conferring the activity of “yol073c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said Yol073c, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Yol073c, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Yol073c, or a functional equivalent or a homolog thereof as depicted in column 8 of Table II, application no. 15, preferably a homolog or functional equivalent as depicted in column 8 of Table II B, application no. 15, and being depicted in the same respective line as said Yol073c, and preferably the activity is increased plastidic, whereby the respective line disclose in column 7 the fine chemical malate.

Accordingly, in one embodiment, the molecule which activity is to be increased in the process of the invention is the gene product with an activity as a “yol073c-protein”, preferably it is the molecule of section (a) or (b) of this paragraph.

In particular, it was observed that in plants, especially in Arabidopsis thaliana, increasing or generating the activity of a gene product plastidic with the activity of a “yol073c-protein”, preferably being encoded by a gene comprising the nucleic acid sequence SEQ ID NO. 137818, preferably the coding region thereof, conferred the production of or the increase in malate compared with the wild type control.

The nucleic acid sequence of Yol160w from Saccharomyces cerevisiae, e.g. as shown in the respective line in column 5 of Table I, application no. 15, has been published in A. And the activity of the gene product thereof is the activity of yol160w-protein.

Accordingly, in one embodiment, the process of the present invention for producing vitamin c and/or threonic acid in a non-human organism, like a microorganism or a plant or a part thereof, comprises increasing or generating the activity of a gene product with the activity of a gene product conferring the activity of “yol160w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical vitamin c and/or threonic acid), application no. 15, preferably the coding region thereof, or a homolog or a fragment thereof, and being depicted in the same respective line as said Yol160w, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Yol160w, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Yol160w, or a functional equivalent or a homolog thereof as depicted in column 8 of Table II, application no. 15, preferably a homolog or functional equivalent as depicted in column 8 of Table II B, application no. 15, and being depicted in the same respective line as said Yol160w, and preferably the activity is increased plastidic, whereby the respective line disclose in column 7 the fine chemical vitamin c and/or threonic acid.

Accordingly, in one embodiment, the molecule which activity is to be increased in the process of the invention is the gene product with an activity as a “yol160w-protein”, preferably it is the molecule of section (a) or (b) of this paragraph.

In particular, it was observed that in plants, especially in Arabidopsis thaliana, increasing or generating the activity of a gene product plastidic with the activity of a “yol160w-protein”, preferably being encoded by a gene comprising the nucleic acid sequence SEQ ID NO. 15179, preferably the coding region thereof, conferred the production of or the increase in vitamin c and/or threonic acid compared with the wild type control.

The nucleic acid sequence of Ypl028w from Saccharomyces cerevisiae, e.g. as shown in the respective line in column 5 of Table I, application no. 15, has been published in A. And the activity of the gene product thereof is the activity of acetyl-CoA acetyltransferase.

Accordingly, in one embodiment, the process of the present invention for producing malate in a non-human organism, like a microorganism or a plant or a part thereof, comprises increasing or generating the activity of a gene product with the activity of a gene product conferring the activity of “acetyl-CoA acetyltransferase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said Ypl028w, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Ypl028w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Ypl028w, or a functional equivalent or a homolog thereof as depicted in column 8 of Table II, application no. 15, preferably a homolog or functional equivalent as depicted in column 8 of Table II B, application no. 15, and being depicted in the same respective line as said Ypl028w, and preferably the activity is increased non-targeted, whereby the respective line disclose in column 7 the fine chemical malate.

Accordingly, in one embodiment, the molecule which activity is to be increased in the process of the invention is the gene product with an activity as a “acetyl-CoA acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyl-CoA acetyltransferase”, preferably being encoded by a gene comprising the nucleic acid sequence SEQ ID NO. 92668, preferably the coding region thereof, conferred the production of or the increase in malate compared with the wild type control.

The nucleic acid sequence of Ypr138c from Saccharomyces cerevisiae, e.g. as shown in the respective line in column 5 of Table I, application no. 15, has been published in A. And the activity of the gene product thereof is the activity of ammonium transporter.

Accordingly, in one embodiment, the process of the present invention for producing vitamin c and/or threonic acid in a non-human organism, like a microorganism or a plant or a part thereof, comprises increasing or generating the activity of a gene product with the activity of a gene product conferring the activity of “ammonium transporter”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical vitamin c and/or threonic acid), application no. 15, preferably the coding region thereof, or a homolog or a fragment thereof, and being depicted in the same respective line as said Ypr138c, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said Ypr138c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said Ypr138c, or a functional equivalent or a homolog thereof as depicted in column 8 of Table II, application no. 15, preferably a homolog or functional equivalent as depicted in column 8 of Table II B, application no. 15, and being depicted in the same respective line as said Ypr138c, and preferably the activity is increased non-targeted, whereby the respective line disclose in column 7 the fine chemical vitamin c and/or threonic acid.

Accordingly, in one embodiment, the molecule which activity is to be increased in the process of the invention is the gene product with an activity as a “ammonium transporter”, preferably it is the molecule of section (a) or (b) of this paragraph.

In particular, it was observed that in plants, especially in Arabidopsis thaliana, increasing or generating the activity of a gene product non-targeted with the activity of a “ammonium transporter”, preferably being encoded by a gene comprising the nucleic acid sequence SEQ ID NO. 93218, preferably the coding region thereof, conferred the production of or the increase in vitamin c and/or threonic acid compared with the wild type control.

The nucleic acid sequence of 47266012_SOYBEAN from Glycine max, e.g. as shown in the respective line in column 5 of Table I, application no. 15, is unpublished. And the activity of the gene product thereof is the activity of 47266012-protein.

Accordingly, in one embodiment, the process of the present invention for producing fumarate in a non-human organism, like a microorganism or a plant or a part thereof, comprises increasing or generating the activity of a gene product with the activity of a gene product conferring the activity of “47266012-protein”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said 47266012_SOYBEAN, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said 47266012_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said 47266012_SOYBEAN, or a functional equivalent or a homolog thereof as depicted in column 8 of Table II, application no. 15, preferably a homolog or functional equivalent as depicted in column 8 of Table II B, application no. 15, and being depicted in the same respective line as said 47266012_SOYBEAN, and preferably the activity is increased non-targeted, whereby the respective line disclose in column 7 the fine chemical fumarate.

Accordingly, in one embodiment, the molecule which activity is to be increased in the process of the invention is the gene product with an activity as a “47266012-protein”, preferably it is the molecule of section (a) or (b) of this paragraph.

In particular, it was observed that in plants, especially in Arabidopsis thaliana, increasing or generating the activity of a gene product non-targeted with the activity of a “47266012-protein”, preferably being encoded by a gene comprising the nucleic acid sequence SEQ ID NO. 15187, preferably the coding region thereof, conferred the production of or the increase in fumarate compared with the wild type control.

The nucleic acid sequence of 47266012_SOYBEAN from Glycine max, e.g. as shown in the respective line in column 5 of Table I, application no. 15, is unpublished. And the activity of the gene product thereof is the activity of 47266012-protein.

Accordingly, in one embodiment, the process of the present invention for producing malate in a non-human organism, like a microorganism or a plant or a part thereof, comprises increasing or generating the activity of a gene product with the activity of a gene product conferring the activity of “47266012-protein”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said 47266012_SOYBEAN, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said 47266012_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said 47266012_SOYBEAN, or a functional equivalent or a homolog thereof as depicted in column 8 of Table II, application no. 15, preferably a homolog or functional equivalent as depicted in column 8 of Table II B, application no. 15, and being depicted in the same respective line as said 47266012_SOYBEAN, and preferably the activity is increased non-targeted, whereby the respective line disclose in column 7 the fine chemical malate.

Accordingly, in one embodiment, the molecule which activity is to be increased in the process of the invention is the gene product with an activity as a “47266012-protein”, preferably it is the molecule of section (a) or (b) of this paragraph.

In particular, it was observed that in plants, especially in Arabidopsis thaliana, increasing or generating the activity of a gene product non-targeted with the activity of a “47266012-protein”, preferably being encoded by a gene comprising the nucleic acid sequence SEQ ID NO. 15187, preferably the coding region thereof, conferred the production of or the increase in malate compared with the wild type control.

The nucleic acid sequence of 47266012_SOYBEAN from Glycine max, e.g. as shown in the respective line in column 5 of Table I, application no. 15, is unpublished. And the activity of the gene product thereof is the activity of 47266012-protein.

Accordingly, in one embodiment, the process of the present invention for producing succinate in a non-human organism, like a microorganism or a plant or a part thereof, comprises increasing or generating the activity of a gene product with the activity of a gene product conferring the activity of “47266012-protein”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said 47266012_SOYBEAN, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said 47266012_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said 47266012_SOYBEAN, or a functional equivalent or a homolog thereof as depicted in column 8 of Table II, application no. 15, preferably a homolog or functional equivalent as depicted in column 8 of Table II B, application no. 15, and being depicted in the same respective line as said 47266012_SOYBEAN, and preferably the activity is increased non-targeted, whereby the respective line disclose in column 7 the fine chemical succinate.

Accordingly, in one embodiment, the molecule which activity is to be increased in the process of the invention is the gene product with an activity as a “47266012-protein”, preferably it is the molecule of section (a) or (b) of this paragraph.

In particular, it was observed that in plants, especially in Arabidopsis thaliana, increasing or generating the activity of a gene product non-targeted with the activity of a “47266012-protein”, preferably being encoded by a gene comprising the nucleic acid sequence SEQ ID NO. 15187, preferably the coding region thereof, conferred the production of or the increase in succinate compared with the wild type control.

The nucleic acid sequence of 49747384_SOYBEAN from Glycine max, e.g. as shown in the respective line in column 5 of Table I, application no. 15, is unpublished. And the activity of the gene product thereof is the activity of 49747384_SOYBEAN-protein.

Accordingly, in one embodiment, the process of the present invention for producing fumarate in a non-human organism, like a microorganism or a plant or a part thereof, comprises increasing or generating the activity of a gene product with the activity of a gene product conferring the activity of “49747384_SOYBEAN-protein”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical fumarate), application no. 15, preferably the coding region thereof, or a homolog or a fragment thereof, and being depicted in the same respective line as said 49747384_SOYBEAN, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said 49747384_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said 49747384_SOYBEAN, or a functional equivalent or a homolog thereof as depicted in column 8 of Table II, application no. 15, preferably a homolog or functional equivalent as depicted in column 8 of Table II B, application no. 15, and being depicted in the same respective line as said 49747384_SOYBEAN, and preferably the activity is increased non-targeted, whereby the respective line disclose in column 7 the fine chemical fumarate.

Accordingly, in one embodiment, the molecule which activity is to be increased in the process of the invention is the gene product with an activity as a “49747384_SOYBEAN-protein”, preferably it is the molecule of section (a) or (b) of this paragraph.

In particular, it was observed that in plants, especially in Arabidopsis thaliana, increasing or generating the activity of a gene product non-targeted with the activity of a “49747384_SOYBEAN-protein”, preferably being encoded by a gene comprising the nucleic acid sequence SEQ ID NO. 69, preferably the coding region thereof, conferred the production of or the increase in fumarate compared with the wild type control.

The nucleic acid sequence of 49747384_SOYBEAN from Glycine max, e.g. as shown in the respective line in column 5 of Table I, application no. 15, is unpublished. And the activity of the gene product thereof is the activity of 49747384_SOYBEAN-protein.

Accordingly, in one embodiment, the process of the present invention for producing malate in a non-human organism, like a microorganism or a plant or a part thereof, comprises increasing or generating the activity of a gene product with the activity of a gene product conferring the activity of “49747384_SOYBEAN-protein”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical malate), application no. 15, preferably the coding region thereof, or a homolog or a fragment thereof, and being depicted in the same respective line as said 49747384_SOYBEAN, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said 49747384_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said 49747384_SOYBEAN, or a functional equivalent or a homolog thereof as depicted in column 8 of Table II, application no. 15, preferably a homolog or functional equivalent as depicted in column 8 of Table II B, application no. 15, and being depicted in the same respective line as said 49747384_SOYBEAN, and preferably the activity is increased non-targeted, whereby the respective line disclose in column 7 the fine chemical malate.

Accordingly, in one embodiment, the molecule which activity is to be increased in the process of the invention is the gene product with an activity as a “49747384_SOYBEAN-protein”, preferably it is the molecule of section (a) or (b) of this paragraph.

In particular, it was observed that in plants, especially in Arabidopsis thaliana, increasing or generating the activity of a gene product non-targeted with the activity of a “49747384_SOYBEAN-protein”, preferably being encoded by a gene comprising the nucleic acid sequence SEQ ID NO. 69, preferably the coding region thereof, conferred the production of or the increase in malate compared with the wild type control.

The nucleic acid sequence of 49747384_SOYBEAN from Glycine max, e.g. as shown in the respective line in column 5 of Table I, application no. 15, is unpublished. And the activity of the gene product thereof is the activity of 49747384_SOYBEAN-protein.

Accordingly, in one embodiment, the process of the present invention for producing succinate in a non-human organism, like a microorganism or a plant or a part thereof, comprises increasing or generating the activity of a gene product with the activity of a gene product conferring the activity of “49747384_SOYBEAN-protein”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said 49747384_SOYBEAN, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said 49747384_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said 49747384_SOYBEAN, or a functional equivalent or a homolog thereof as depicted in column 8 of Table II, application no. 15, preferably a homolog or functional equivalent as depicted in column 8 of Table II B, application no. 15, and being depicted in the same respective line as said 49747384_SOYBEAN, and preferably the activity is increased non-targeted, whereby the respective line disclose in column 7 the fine chemical succinate.

Accordingly, in one embodiment, the molecule which activity is to be increased in the process of the invention is the gene product with an activity as a “49747384_SOYBEAN-protein”, preferably it is the molecule of section (a) or (b) of this paragraph.

In particular, it was observed that in plants, especially in Arabidopsis thaliana, increasing or generating the activity of a gene product non-targeted with the activity of a “49747384_SOYBEAN-protein”, preferably being encoded by a gene comprising the nucleic acid sequence SEQ ID NO. 69, preferably the coding region thereof, conferred the production of or the increase in succinate compared with the wild type control.

The nucleic acid sequence of 49747384_SOYBEAN from Glycine max, e.g. as shown in the respective line in column 5 of Table I, application no. 15, is unpublished. And the activity of the gene product thereof is the activity of 49747384_SOYBEAN-protein.

Accordingly, in one embodiment, the process of the present invention for producing vitamin c and/or threonic acid in a non-human organism, like a microorganism or a plant or a part thereof, comprises increasing or generating the activity of a gene product with the activity of a gene product conferring the activity of “49747384_SOYBEAN-protein”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical vitamin c and/or threonic acid), application no. 15, preferably the coding region thereof, or a homolog or a fragment thereof, and being depicted in the same respective line as said 49747384_SOYBEAN, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said 49747384_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said 49747384_SOYBEAN, or a functional equivalent or a homolog thereof as depicted in column 8 of Table II, application no. 15, preferably a homolog or functional equivalent as depicted in column 8 of Table II B, application no. 15, and being depicted in the same respective line as said 49747384_SOYBEAN, and preferably the activity is increased non-targeted, whereby the respective line disclose in column 7 the fine chemical vitamin c and/or threonic acid.

Accordingly, in one embodiment, the molecule which activity is to be increased in the process of the invention is the gene product with an activity as a “49747384_SOYBEAN-protein”, preferably it is the molecule of section (a) or (b) of this paragraph.

In particular, it was observed that in plants, especially in Arabidopsis thaliana, increasing or generating the activity of a gene product non-targeted with the activity of a “49747384_SOYBEAN-protein”, preferably being encoded by a gene comprising the nucleic acid sequence SEQ ID NO. 69, preferably the coding region thereof, conferred the production of or the increase in vitamin c and/or threonic acid compared with the wild type control.

The nucleic acid sequence of 51340801_CANOLA from Brassica napus, e.g. as shown in the respective line in column 5 of Table I, application no. 15, is unpublished. And the activity of the gene product thereof is the activity of pyruvate kinase.

Accordingly, in one embodiment, the process of the present invention for producing citramalate in a non-human organism, like a microorganism or a plant or a part thereof, comprises increasing or generating the activity of a gene product with the activity of a gene product conferring the activity of “pyruvate kinase”, especially from Brassica napus or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said 51340801_CANOLA, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said 51340801_CANOLA, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said 51340801_CANOLA, or a functional equivalent or a homolog thereof as depicted in column 8 of Table II, application no. 15, preferably a homolog or functional equivalent as depicted in column 8 of Table II B, application no. 15, and being depicted in the same respective line as said 51340801_CANOLA, and preferably the activity is increased plastidic, whereby the respective line disclose in column 7 the fine chemical citramalate.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 15532, preferably the coding region thereof, conferred the production of or the increase in citramalate compared with the wild type control.

The nucleic acid sequence of 51340801_CANOLA from Brassica napus, e.g. as shown in the respective line in column 5 of Table I, application no. 15, is unpublished. And the activity of the gene product thereof is the activity of pyruvate kinase.

Accordingly, in one embodiment, the process of the present invention for producing succinate in a non-human organism, like a microorganism or a plant or a part thereof, comprises increasing or generating the activity of a gene product with the activity of a gene product conferring the activity of “pyruvate kinase”, especially from Brassica napus or its functional equivalent or its homolog, e.g. the increase of
(a) a gene product of a gene comprising the nucleic acid molecule as shown in the respective line in column 5 of Table I (whereby the respective line disclose in column 7 the fine chemical succinate), application no. 15, preferably the coding region thereof, or a homolog or a fragment thereof, and being depicted in the same respective line as said 51340801_CANOLA, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said 51340801_CANOLA, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said 51340801_CANOLA, or a functional equivalent or a homolog thereof as depicted in column 8 of Table II, application no. 15, preferably a homolog or functional equivalent as depicted in column 8 of Table II B, application no. 15, and being depicted in the same respective line as said 51340801_CANOLA, and preferably the activity is increased plastidic, whereby the respective line disclose in column 7 the fine chemical succinate.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 15532, preferably the coding region thereof, conferred the production of or the increase in succinate compared with the wild type control.

The nucleic acid sequence of 59582753_SOYBEAN from Glycine max, e.g. as shown in the respective line in column 5 of Table I, application no. 15, is unpublished. And the activity of the gene product thereof is the activity of glucose-6-phosphate 1-dehydrogenase.

Accordingly, in one embodiment, the process of the present invention for producing citramalate in a non-human organism, like a microorganism or a plant or a part thereof, comprises increasing or generating the activity of a gene product with the activity of a gene product conferring the activity of “glucose-6-phosphate 1-dehydrogenase”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical citramalate), application no. 15, preferably the coding region thereof, or a homolog or a fragment thereof, and being depicted in the same respective line as said 59582753_SOYBEAN, or a functional equivalent or a homolog thereof as shown in column 8 of Table I, application no. 15, preferably the coding region thereof, particularly a homolog or functional equivalent as shown in column 8 of Table I B, application no. 15, and being depicted in the same respective line as said 59582753_SOYBEAN, and preferably the activity is increased plastidic, or
(b) a polypeptide comprising a polypeptide, a consensus sequence or at least a polypeptide motif as shown in the respective line in column 5 of Table II or in column 8 of Table IV, application no. 15, respectively, and being depicted in the same respective line as said 59582753_SOYBEAN, or a functional equivalent or a homolog thereof as depicted in column 8 of Table II, application no. 15, preferably a homolog or functional equivalent as depicted in column 8 of Table II B, application no. 15, and being depicted in the same respective line as said 59582753_SOYBEAN, and preferably the activity is increased plastidic, whereby the respective line disclose in column 7 the fine chemical citramalate.

Accordingly, in one embodiment, the molecule which activity is to be increased in the process of the invention is the gene product with an activity as a “glucose-6-phosphate 1-dehydrogenase”, preferably it is the molecule of section (a) or (b) of this paragraph.

In particular, it was observed that in plants, especially in Arabidopsis thaliana, increasing or generating the activity of a gene product plastidic with the activity of a “glucose-6-phosphate 1-dehydrogenase”, preferably being encoded by a gene comprising the nucleic acid sequence SEQ ID NO. 16883, preferably the coding region thereof, conferred the production of or the increase in citramalate compared with the wild type control.

[0105.1.7.15] to [0107.1.7.15] for the disclosure of these paragraphs see [0105.1.7.7] to [0107.1.7.7] above

A protein having an activity conferring an increase in the amount or level of the fine chemical citramalate, citrate, fumarate, glycerate, malate, oxalate, pyruvate, succinate, or vitamin c and/or threonic acid, upon targeting to the plastids or mitochondria or upon non-targeting, preferably has the structure of the respective polypeptide described herein, in particular of the polypeptides comprising the consensus sequence or at least one polypeptide motifs as shown in the respective line in Table IV, application no. 15, column 8 or of the polypeptide comprising an amino acid sequence as disclosed in the respective line in Table II, application no. 15, columns 5 or 8, or homologs or fragments thereof as described herein, or is encoded by the nucleic acid molecule characterized herein or the nucleic acid molecule according to the invention, for example by the nucleic acid molecule as shown in the respective line in Table I, application no. 15, columns 5 or 8, preferably the coding region thereof, or homologs or fragments thereof and has the herein mentioned activity.

for the disclosure of this paragraph see [0109.1.7.7] above

for the disclosure of this paragraph see [0110.1.7.7] above

In one embodiment, the process of the present invention comprises one or more of the following steps
(a) stabilizing a protein conferring the generation or increased expression of a protein encoded by the nucleic acid molecule of the invention or of the polypeptide of the invention, e.g. of a polypeptide having the activity selected from the group consisting of 2-C-methyl-D-erythritol 4-phosphate cytidylyltransferase, 2-isopropylmalate synthase, 2-oxoglutarate dehydrogenase E1 subunit, 30S ribosomal protein S3, 47266012-protein, 49747384_SOYBEAN-protein, 50S ribosomal protein L14, ABC transporter ATP binding component, ABC transporter permease protein, acetyl-CoA acetyltransferase, acetyl-CoA hydrolase, acetyltransferase, acyl-CoA dehydrogenase, acyl-CoA synthase, adenylate kinase, adenylylsulfate kinase, agmatinase, amino-acid acetyltransferase, aminotransferase, ammonium transporter, arginine N-succinyltransferase, aspartase, AO g13930-protein, ATP-binding component of a transport system, auxin response factor, AX653549-protein, AY087308-protein, b0801-protein, b1003-protein, b1285-protein, b1425-protein, b1445-protein, b1522-protein, b1670-protein, b1672-protein, b1742-protein, b1898-protein, b2399-protein, b3777-protein, b3791-protein, b3814-protein, b3817-protein, b3989-protein, b4029-protein, b4050-protein, b4056-protein, beta-hydroxylase, betaine aldehyde dehydrogenase, bifunctional purine biosynthesis protein, branched chain aminotransferase, calcium-dependent protein kinase, cation-transporting ATPase, CBL-interacting protein kinase, CDP-diacylglycerol-glycerol-3-phosphate 3-phosphatidyltransferase, CDP-diacylglycerol-serine O-phosphatidyltransferase, CDP-glucose-4,6-dehydratase, citrate synthase, coproporphyrinogen III oxidase, CTP synthase, cystathionine beta-lyase, cysteine synthase A, cysteine synthase B, dATP pyrophosphohydrolase, dihydrolipoamide dehydrogenase, DnaJ-like chaperone, electron transfer flavoprotein subunit beta, electron transport complex protein, eukaryotic translation initiation factor 5, Fe—S subunit of oxidoreductase, flagellar M-ring protein, fumarate hydratase, gamma-glutamyltranspeptidase, gibberellin 20-oxidase, glucose dehydrogenase, glucose-1-phosphate cytidylyltransferase, glucose-6-phosphate 1-dehydrogenase, glutamate dehydrogenase, glutamate-ammonia-ligase, glutaredoxin, glycerate kinase, glycerol kinase, Glycine cleavage system T aminomethyltransferase, GM02LC17485-protein, GM02LC5744-protein, heat shock protein, heat shock transcription factor, hexose transporter, hydrolase, isochorismate synthase, isocitrate dehydrogenase, isocitrate lyase, leucyl/phenylalanyl-tRNA-protein transferase, lipopolysaccharide core biosynthesis protein, lipoprotein precursor, lysine decarboxylase, malate dehydrogenase, malonyl CoA-acyl carrier protein transacylase, membrane protein, membrane transport protein, methylglyoxal synthase, methylglyoxalase, methyltransferase, monothiol glutaredoxin, monthiol glutaredoxin, multiple antibiotic resistance protein, murein transglycosylase, NADH-quinone oxidoreductase subunit, O-antigen chain length determinant, oxidoreductase, oxidoreductase subunit, oxireductase, phenylacetic acid degradation protein, phenylalanine ammonia-lyase, phosphatase, phosphate import ATP-binding protein, photosystem I reaction centre subunit, photosystem II protein, polygalacturonase, protease, protein phosphatase, purine-nucleoside phosphorylase, pyruvate carboxylase, pyruvate kinase, recombinase A, ribulokinase, RNA polymerase sigma factor, sec-independent protein translocase, Sec-independent protein translocase subunit, slr1170-protein, thioredoxin, thioredoxin family protein, thioredoxin reductase, threonine efflux protein, threonine synthase, transcription factor, transcriptional regulator, transcriptional regulator protein, translation initiation factor subunit, TTC1386-protein, xanthine dehydrogenase, XM_{—}473199-protein, ycrl 02c-protein, YDL159W-A-protein, ygl237c-protein, YGR193C-protein, ygr221 c-protein, yhr131c-protein, yhr207c-protein, YKL038W-protein, YKL140W-protein, ylr065c-protein, ylr326w-protein, yml084w-protein, yol073c-protein, yol160w-protein, zinc finger protein, and zinc transporter, or of a polypeptide as indicated in the respective line in Table II, application no. 15, columns 5 or 8, or its homologs or fragments, and conferring the production of or an increase in citramalate, citrate, fumarate, glycerate, malate, oxalate, pyruvate, succinate, or vitamin c and/or threonic acid, respectively; and/or
(b) stabilizing a mRNA conferring the generation or increased expression of a FCRP, e.g. protein encoded by the nucleic acid molecule of the invention or its homologs or fragments, or of a mRNA encoding the polypeptide of the present invention having the hereinmentioned activity selected from the group consisting of said activities mentioned in (a) and conferring the production of or an increase in citramalate, citrate, fumarate, glycerate, malate, oxalate, pyruvate, succinate, or vitamin c and/or threonic acid, respectively; and/or
(c) increasing the specific activity of a protein conferring the increased expression of a FCRP, e.g. a protein encoded by the nucleic acid molecule of the invention or of the polypeptide of the present invention having herein-mentioned citramalate, citrate, fumarate, glycerate, malate, oxalate, pyruvate, succinate, or vitamin c and/or threonic acid generating or increasing activity, respectively, e.g. of a polypeptide having the activity of a protein as indicated in the respective line in Table II, application no. 15, columns 5 or 8, or its homologs or fragments, or decreasing the inhibitory regulation of the polypeptide of the invention; and/or
(d) generating or increasing the expression of an endogenous or artificial transcription factor mediating the expression of a protein conferring the generation or increased expression of a FCRP, e.g. a protein encoded by the nucleic acid molecule of the invention or of the polypeptide of the invention having herein-mentioned activity selected from the group consisting of said activities mentioned in (a) and conferring a citramalate, citrate, fumarate, glycerate, malate, oxalate, pyruvate, succinate, or vitamin c and/or threonic acid increasing activity, respectively, e.g. of a polypeptide having the activity of a protein as indicated in the respective line in Table II, application no. 15, columns 5 or 8 or its homologs; and/or
(e) stimulating activity of a protein conferring the increased expression of a FCRP, e.g. a protein encoded by the nucleic acid molecule of the present invention or a polypeptide of the present invention having herein-mentioned activity selected from the group consisting of said activities mentioned in (a) and conferring a citramalate, citrate, fumarate, glycerate, malate, oxalate, pyruvate, succinate, or vitamin c and/or threonic acid increasing activity, respectively, e.g. of a polypeptide having the activity of a protein as indicated in the respective line in Table II, application no. 15, columns 5 or 8, or its homologs or fragments, by adding one or more exogenous inducing factors to the non-human organism or parts thereof; and/or
(f) expressing a transgenic gene encoding a protein conferring the increased expression of a FCRP, e.g. a polypeptide encoded by the nucleic acid molecule of the present invention or a polypeptide of the present invention, having herein-mentioned activity selected from the group consisting of said activities mentioned in (a) and conferring a citramalate, citrate, fumarate, glycerate, malate, oxalate, pyruvate, succinate, or vitamin c and/or threonic acid increasing activity, respectively, e.g. of a polypeptide having the activity of a protein as indicated in the respective line in Table II, application no. 15, columns 5 or 8, or its homologs or fragments, and/or
(g) increasing the copy number of a gene conferring the increased expression of a nucleic acid molecule encoding a FCRP, e.g. a polypeptide encoded by the nucleic acid molecule of the invention or the polypeptide of the invention having herein-mentioned activity selected from the group consisting of said activities mentioned in (a) and conferring a citramalate, citrate, fumarate, glycerate, malate, oxalate, pyruvate, succinate, or vitamin c and/or threonic acid increasing activity, respectively, e.g. of a polypeptide having the activity of a protein as indicated in the respective line in Table II, application no. 15, columns 5 or 8, or its homologs or fragments; and/or
(h) increasing the expression of the endogenous gene encoding the FCRP, polypeptide of the invention, e.g. a polypeptide encoded by the nucleic acid molecule of the invention or the polypeptide of the invention having herein-mentioned activity selected from the group consisting of said activities mentioned in (a) and conferring a citramalate, citrate, fumarate, glycerate, malate, oxalate, pyruvate, succinate, or vitamin c and/or threonic acid; increasing activity, respectively, e.g. of a polypeptide having the activity of a protein as indicated in the respective line in Table II, application no. 15, columns 5 or 8, or its homologs or fragments, by adding positive expression or removing negative expression elements, e.g. homologous recombination can be used to either introduce positive regulatory elements like for plants the 35S enhancer into the promoter or to remove repressor elements form regulatory regions. Further gene conversion methods can be used to disrupt repressor elements or to enhance to activity of positive elements. Positive elements can be randomly introduced in plants by T-DNA or transposon mutagenesis and lines can be identified in which the positive elements have be integrated near to a gene of the invention, the expression of which is thereby enhanced; and/or
(i) modulating growth conditions of the non-human organism in such a manner, that the expression or activity of the gene encoding the FCRP, e.g. a protein of the invention or the protein itself is enhanced, for example microorganisms or plants can be grown for example under a higher temperature regime leading to an enhanced expression of heat shock proteins, which can lead to an enhanced citramalate, citrate, fumarate, glycerate, malate, oxalate, pyruvate, succinate, or vitamin c and/or threonic acid production, respectively; and/or
(j) selecting of non-human organisms with especially high activity of the proteins of the invention from natural or from mutagenized resources and breeding them into the target organisms, e.g. the elite crops; and/or
(k) directing a protein encoded by the nucleic acid molecule of the invention or of the polypeptide of the present invention having herein-mentioned activity selected from the group consisting of said activities mentioned in (a) and conferring a citramalate, citrate, fumarate, glycerate, malate, oxalate, pyruvate, succinate, or vitamin c and/or threonic acid increasing activity, respectively, e.g. of a polypeptide having the activity of a protein as indicated in the respective line in Table II, application no. 15, columns 5 or 8, or its homologs or fragments, if for the corresponding nucleic acid molecule in the respective line in column 6 of Table I the term “plastidic” is indicated, to the plastids by the addition of a plastidial targeting sequence or if for the corresponding nucleic acid molecule in the respective line in column 6 of Table I the term “mitochondric” is indicated, to the mitochondria by the addition of a mitochondrial targeting sequence; and/or
(l) generating the expression of a protein encoded by the nucleic acid molecule of the invention or of the polypeptide of the present invention having herein-mentioned activity selected from the group consisting of said activities mentioned in (a) and conferring a citramalate, citrate, fumarate, glycerate, malate, oxalate, pyruvate, succinate, or vitamin c and/or threonic acid increasing activity, respectively, e.g. of a polypeptide having the activity of a protein as indicated in the respective line in Table II, application no. 15, columns 5 or 8, or its homologs or fragments, if for the corresponding nucleic acid molecule in the respective line in column 6 of Table I the term “plastidic” or “mitochondric” is indicated, in these organelles by the stable or transient transformation, advantageously stable transformation, of organelles, preferably plastids or mitochondria, with an inventive nucleic acid sequence preferably in form of an expression cassette containing said sequence leading to the expression of the nucleic acids or polypeptides of the invention in the respective organelle; and/or
(m) generating the expression of a protein encoded by the nucleic acid molecule of the invention or of the polypeptide of the present invention having herein-mentioned activity selected from the group consisting of said activities mentioned in (a) and conferring a citramalate, citrate, fumarate, glycerate, malate, oxalate, pyruvate, succinate, and/or vitamin c and/or threonic acid increasing activity, respectively, e.g. of a polypeptide having the activity of a protein as indicated in the respective line in Table II, application no. 15, columns 5 or 8, or its homologs or fragments, if for the corresponding nucleic acid molecule in the respective line in column 6 of Table I the term “plastidic” or “mitochondric” is indicated, in these organelles by integration of a nucleic acid of the invention into the genome of the respective organelle under control of preferable a promoter selective for the respective organelle.

Preferably, said mRNA is the coding region of the nucleic acid molecule of the present invention and/or the protein conferring the increased expression of a protein encoded by the coding region of the nucleic acid molecule of the present invention alone or linked to a transit nucleic acid sequence or transit peptide encoding nucleic acid sequence or the polypeptide having the herein mentioned activity, e.g. conferring the generation of or increase of citramalate, citrate, fumarate, glycerate, malate, oxalate, pyruvate, succinate, or vitamin c and/or threonic acid, respectively, after increasing the expression or activity of the encoded polypeptide, non-targeted or in organelles such as plastids and/or mitochondria, preferably plastids, or having the activity of a polypeptide having an activity as the protein as shown in the respective line in Table II, application no. 15, column 3, or its homologs. Preferably the increase of citramalate, citrate, fumarate, glycerate, malate, oxalate, pyruvate, succinate, or vitamin c and/or threonic acid, respectively, takes place non-targeted or in plastids and/or mitochondria, preferably non-targeted or in plastids.

[0113.1.7.15] to [0122.1.7.15] for the disclosure of these paragraphs see [0113.1.7.7] to [0122.1.7.7] above

The activation of an endogenous polypeptide having above-mentioned activity, e.g. having the activity of a protein as shown in the respective line in Table II, application no. 15, column 3 or of the polypeptide of the invention, e.g. conferring the increase of the fine chemical citramalate, citrate, fumarate, glycerate, malate, oxalate, pyruvate, succinate, or vitamin c and/or threonic acid, respectively, by increase of expression or activity in the cytoplasm, and/or in the cytosol, and/or in an organelle, such as plastids or mitochondria, can also be increased by introducing a synthetic transcription factor, which binds close to the coding region of the gene encoding the protein as shown in the respective line in Table II, application no. 15, column 5 or 8, or homologs or fragments thereof, and activates its transcription. A chimeric zinc finger protein can be constructed, which comprises a specific DNA-binding domain and an activation domain as e.g. the VP16 domain of Herpes Simplex virus. The specific binding domain can bind to the regulatory region of the gene encoding the protein as shown in the respective line in Table II, application no. 15, column 5 or 8, or homologs or fragments thereof. The expression of the chimeric transcription factor in a non-human organism, in particular in a plant, leads to a specific expression of the protein as shown in the respective line in Table II, application no. 15, column 5 or 8, or homologs or fragments thereof, see e.g. in W001/52620, Oriz, Proc. Natl. Acad. Sci. USA 99, 13290 (2002), or Guan, Proc. Natl. Acad. Sci. USA 99, 13296 (2002).

[0124.1.7.15] to [0127.1.7.15] for the disclosure of these paragraphs see [0124.1.7.7] to [0127.1.7.7] above

Owing to the introduction of a gene or a plurality of genes conferring the expression of the nucleic acid molecule of the invention or the polypeptide of the invention, for example the nucleic acid construct mentioned below, or encoding the protein as shown in the respective line in Table II, application no. 15, column 5 or 8, or homologs or fragments thereof, into a non-human organism alone or in combination with other genes, it is possible not only to increase the biosynthetic flux towards the end product, but also to increase, modify or create de novo an advantageous, preferably novel metabolites composition in the non-human organism, e.g. an advantageous composition comprising a higher content of (from a viewpoint of nutritional physiology limited) citramalate, citrate, fumarate, glycerate, malate, oxalate, pyruvate, succinate, or vitamin c and/or threonic acid and if desired other substrate or product of the citric acid cycle, or a derivate of one of the substrates or products, and/or other metabolites, in free or bound form.

for the disclosure of this paragraph see [0129.1.7.7] above

Accordingly, in one embodiment, the process according to the invention relates to a process, which comprises:
(a) providing a non-human organism, preferably a microorganism, a plant cell, a plant tissue, a plant or a part thereof, more preferably a microorganism, a plant tissue, a plant or a part thereof;
(b) increasing the activity of a protein as shown in the respective line in Table II, application no. 15, column 3 or of a polypeptide being encoded by the respective nucleic acid molecule of the present invention and described below, e.g. conferring an increase of the fine chemical citramalate, citrate, fumarate, glycerate, malate, oxalate, pyruvate, succinate, or vitamin c and/or threonic acid, respectively, in the non-human organism, preferably in the microorganism, the plant cell, the plant tissue, the plant or a part thereof, more preferably a microorganism, a plant tissue, a plant or a part thereof, especially cytoplasmic or in an organelle, like plastids or mitochondria,
(c) growing the non-human organism, preferably the microorganism, the plant cell, the plant tissue, the plant or a part thereof, under conditions which permit the production of said chemical in the non-human organism, preferably the microorganism, the plant cell, the plant tissue, the plant or a part thereof; and
(d) if desired, recovering, optionally isolating, said fine chemical, in free and/or bound form, and, optionally further free and/or bound substrate or product of the citric acid cycle, or at least one derivate of one of the substrates or products, and/or other metabolites synthesized by the non-human organism, the microorganism, the plant cell, the plant tissue, the plant or a part thereof.

[0131.1.7.15] to [0133.1.7.15] for the disclosure of these paragraphs see [0131.1.7.7] to [0133.1.7.7] above

The non-human organism, in particular the microorganism, plant or part thereof, is advantageously grown in such a way that it is not only possible to recover, if desired to isolate the free or bound fine chemical or the free and bound fine chemical but as option it is also possible to produce, recover and, if desired to isolate, other free or/and bound vitamins and/or other metabolites.

The organisms are harvested in a manner known per se and appropriate for the particular organism. Microorganisms such as bacteria, mosses, yeasts and fungi or plant cells which are cultured in liquid media by fermentation may be removed, for example, by centrifugation, decanting or filtration. Plants are grown on solid media in a manner known per se and harvested accordingly.

The organisms, its cells or a part thereof or the recovered, and if desired isolated fine chemical citramalate, citrate, fumarate, glycerate, malate, oxalate, pyruvate, succinate, or vitamin c and/or threonic acid or compounds comprising said fine chemical citramalate, citrate, fumarate, glycerate, malate, oxalate, pyruvate, succinate, or vitamin c and/or threonic acid can then be processed further directly into foodstuffs or animal feeds or for other applications, for example according to the disclosures made in WO/2005/083093 or WO2005/012316, which are expressly incorporated herein by reference.

[0135.1.7.15] to [0139.1.7.15] for the disclosure of these paragraphs see [0135.1.7.7] to [0139.1.7.7] above

In another embodiment, the invention relates to a (isolated) nucleic acid molecule, which comprises a nucleic acid molecule selected from the group consisting of:
(a) a nucleic acid molecule encoding a polypeptide shown in Table II, application no. 15, column 5 or 8, preferably shown in Table II B, application no. 15, column 8, or a homolog or a fragment thereof;
(b) a nucleic acid molecule shown in Table I, application no. 15, column 5 or 8, preferably shown in Table I B, application no. 15, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 15, preferably in column 8 of Table II B, application no. 15;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 15, preferably shown in column 8 of Table I B, application no. 15, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 15;
(i) a nucleic acid molecule encoding a polypeptide having the activity represented by a respective protein as depicted in column 5 of Table II, application no. 15;
(j) a nucleic acid molecule comprising a polynucleotide which is obtained by amplifying a cDNA library or a genomic library using the primers in Table III, application no. 15, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j) ;
or a nucleic acid molecule comprising a sequence which is complementary thereto.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 15.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II application no. 15 and confers a generation or an increase of the respective fine chemical in a non-human organism or a part thereof as compared to a corresponding wild-type non-human organism or part thereof.

In another embodiment, the invention relates to a (isolated) nucleic acid molecule, which comprises a nucleic acid molecule selected from the group consisting of:
(a) a nucleic acid molecule encoding a polypeptide shown in Table II, column 5 or 8, application no. 15, preferably shown in Table II A, application no. 15, in column 5 or in Table II A, application no. 15, column 8 or in Table II B, application no. 15, column 8, or a homolog or a fragment thereof;
(b) a nucleic acid molecule shown in Table I, application no. 15, column 5 or 8, preferably shown in Table I A, application no. 15, in column 5 or in Table I A, application no. 15, column 8 or in Table I B, application no. 15, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 15, preferably shown in Table II A, application no. 15, in column 5 or in Table II A, application no. 15, column 8 or in Table II B, application no. 15, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 15, preferably shown in Table I A, application no. 15, in column 5 or in Table I A, application no. 15, column 8 or in Table I B column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 15;
(i) a nucleic acid molecule encoding a polypeptide having the activity represented by a respective protein as depicted in column 5 of Table II, application no. 15;
(j) a nucleic acid molecule comprising a polynucleotide which is obtained by amplifying a cDNA library or a genomic library using the primers in Table III, application no. 15, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt,500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto;
   whereby, preferably, the nucleic acid molecule according to (a), (b), (c), (d), (e), (f), (g), (h), (i), (j) or (k) distinguishes over the sequence depicted in Table I A and/or I B, application no. 15, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 15, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 15, column 5 or 8, or the coding region thereof, respectively. In a further embodiment the nucleic acid molecule does not encode the polypeptide sequence shown in Table II A and/or II B, application no. 15, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 15, column 5 or 8 and does not encode a protein of the sequence shown in Table II A and/or II B, application no. 15, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 15, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 15, column 5 or 8 but less than 100%, preferably less than 99.999%, 99.99%, 99.9%, 99%, 98%, 97%, 96% or 95% identical to the sequence shown in Table II A and/or II B, application no. 15, column 5 or 8.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 15.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II, application no. 15, and confers a generation or an increase of the respective fine chemical in a non-human organism or a part thereof as compared to a corresponding wild-type non-human organism or part thereof.

[0144.1.7.15] to [0155.1.7.15] for the disclosure of these paragraphs see [0144.1.7.7] to [0155.1.7.7] above

In a preferred embodiment a nucleic acid construct, for example an expression cassette, comprises upstream, i.e. at the 5′ end of the encoding sequence, a promoter and downstream, i.e. at the 3′ end, a polyadenylation signal and optionally other regulatory elements which are operably linked to the intervening encoding sequence with one of the nucleic acids as depicted in the respective line in Table I, application no. 15, column 5 or 8, preferably the coding region thereof, or a homolog or a fragment thereof. By an operable linkage is meant the sequential arrangement of promoter, encoding sequence, terminator, optionally other regulatory elements and optionally targeting sequences in such a way that each of the regulatory elements/targeting sequence can fulfill its function in the expression of the encoding sequence in due manner. In one embodiment the sequences preferred for operable linkage are targeting sequences for ensuring subcellular localization in organelles, like plastids or mitochondria.

However, targeting sequences for ensuring subcellular localization in the endoplasmic reticulum (=ER), in the nucleus, in oil corpuscles or other compartments may also be employed as well as translation promoters such as the 5′ lead sequence in tobacco mosaic virus (Gallie et al., Nucl. Acids Res. 15, 8693 (1987)).

for the disclosure of this paragraph see [0157.1.7.7] above

In an embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 15, column 5 or 8, preferably shown in Table II A, application no. 15, column 5, or in Table II A, application no. 15, column 8, or in Table II B, application no. 15, column 8, or a homolog or a fragment thereof;
   (b) a nucleic acid molecule shown in Table I, application no. 15, column 5 or 8, preferably shown in Table I A, application no. 15, column 5, or in Table I A, application no. 15, column 8, or in Table I B, application no. 15, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 15, preferably shown in Table II A, application no. 15, column 5, or in Table II A, application no. 15, column 8, or in Table II B, application no. 15, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nuoleic acid molecule sequence of a polynucleotide comprising the nucleic acid molecule shown in column 5 or 8 of Table I, application no. 15, preferably shown in Table I A, application no. 15, column 5, or in Table I A, application no. 15, column 8, or in Table I B, application no. 15, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 15;
   (i) a nucleic acid molecule encoding a polypeptide having the activity represented by a respective protein as depicted in column 5 of Table II, application no. 15;
   (j) a nucleic acid molecule comprising a polynucleotide which is obtained by amplifying a cDNA library or a genomic library using the primers in Table III, application no. 15, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto.
      and, optionally
3) nucleic acid molecule encoding a transit peptide, preferably encoding a plastidal transit peptide or a mitochondrial transit peptide;
   which are operable linked.
   (All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 15.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 15 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 15, in the same line as of the respective nucleic acid molecule according to 2) “plastidic” is depicted, or encoding a mitochondrial transit peptide, especially in case in column 6 of Table I, application no. 15, in the same line as of the respective nucleic acid molecule according to 2) “mitochondric” is depicted.

In another embodiment thereof the expression cassette does not comprise 3) a nucleic acid molecule encoding a transit peptide, especially in case in column 6 of Table I, application no. 15, in the same line as of the respective nucleic acid molecule according to 2) “non-targeted” is depicted.

In a further embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 15, column 5 or 8, preferably shown in Table II A, application no. 15, column 5, or in Table II A, application no. 15, column 8, or in Table II B, application no. 15, column 8, or a homolog or a fragment thereof;
   (b) a nucleic acid molecule shown in Table I, application no. 15, column 5 or 8, preferably shown in Table I A, application no. 15, column 5, or in Table I A, application no. 15, column 8, or in Table I B, application no. 15, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 15, preferably shown in Table II A, application no. 15, column 5, or in Table II A, application no. 15, column 8, or in Table II B, application no. 15, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 15, preferably shown in Table I A, application no. 15, column 5, or in Table I A, application no. 15, column 8,or in Table I B, application no. 15, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 15;
   (i) a nucleic acid molecule encoding a polypeptide having the activity represented by a respective protein as depicted in column 5 of Table II, application no. 15;
   (j) a nucleic acid molecule comprising a polynucleotide which is obtained by amplifying a cDNA library or a genomic library using the primers in Table III, application no. 15, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, optionally
3) nucleic acid molecule encoding a transit peptide, preferably encoding a plastidal transit peptide or a mitochondrial transit peptide;
   which are operable linked and
   whereby, preferably, the nucleic acid molecule according to 2 (a), (b), (c), (d), (e), (f), (g), (h), (i), (j) or (k) distinguishes over the sequence depicted in Table I A and/or I B, application no. 15, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention according to 2) does not consist of the sequence shown in Table

I A and/or I B, application no. 15, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 15, column 5 or 8, or the coding region thereof, respectively. In a further embodiment the nucleic acid molecule does not encode the polypeptide sequence shown in Table II A and/or II B, application no. 15, column 5 or 8 but homologs thereof.

Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 15, column 5 or 8 and does not encode a protein of the sequence shown in Table II A and/or II B, application no. 15, column 5 or 8. Accordingly, in one embodiment, the protein encoded by a sequence of a nucleic acid according to 2(a), (b), (c), (d), (e), (f), (g), (h), (i), (j) or (k) does not consist of the sequence shown in Table I A and/or I B, application no. 15, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 15, column 5 or 8 but less than 100%, 99.999%, 99.99%, 99.9%, 99%, 98%, 97%, 96% or 95% identical to the sequence shown in Table II A and/or II B, application no. 15, column 5 or 8.

(All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 15.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 15 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 15, in the same line as of the respective nucleic acid molecule according to 2) “plastidic” is depicted, or encoding a mitochondrial transit peptide, especially in case in column 6 of Table I, application no. 15, in the same line as of the respective nucleic acid molecule according to 2) “mitochondric” is depicted.

In another embodiment thereof the expression cassette does not comprise 3) a nucleic acid molecule encoding a transit peptide, especially in case in column 6 of Table I, application no. 15, in the same line as of the respective nucleic acid molecule according to 2) “non-targeted” is depicted.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17451, or a homolog or fragment thereof, or a homolog thereof being depicted in Table I, application no. 15, column 8, in the same line as SEQ ID NO. 17451, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 17451 is depicted, plastidic is mentioned, or a mitochondrial transit peptide in case in Table I, application no. 15, in column 6 in the same line as SEQ ID NO. 17451 is depicted, mitochondric is mentioned; which is operable linked, too.

In another embodiment thereof the expression cassette does not comprise an additional transit peptide in case in Table I, application no. 15, in column 6 in the same line as SEQ ID NO. 17451 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 133001, or a homolog or fragment thereof, or a homolog thereof being depicted in Table I, application no. 15, column 8, in the same line as SEQ ID NO. 133001, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 133001 is depicted, plastidic is mentioned, or a mitochondrial transit peptide in case in Table I, application no. 15, in column 6 in the same line as SEQ ID NO. 133001 is depicted, mitochondric is mentioned; which is operable linked, too.

In another embodiment thereof the expression cassette does not comprise an additional transit peptide in case in Table I, application no. 15, in column 6 in the same line as SEQ ID NO. 133001 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17968, or a homolog or fragment thereof, or a homolog thereof being depicted in Table I, application no. 15, column 8, in the same line as SEQ ID NO. 17968, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 17968 is depicted, plastidic is mentioned, or a mitochondrial transit peptide in case in Table I, application no. 15, in column 6 in the same line as SEQ ID NO. 17968 is depicted, mitochondric is mentioned; which is bpenlithBlireketIoditment thereof the expression cassette does not comprise an additional transit peptide in case in Table I, application no. 15, in column 6 in the same line as SEQ ID NO. 17968 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 18070, or a homolog or fragment thereof, or a homolog thereof being depicted in Table I, application no. 15, column 8, in the same line as SEQ ID NO. 18070, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 18070 is depicted, plastidic is mentioned, or a mitochondrial transit peptide in case in Table I, application no. 15, in column 6 in the same line as SEQ ID NO. 18070 is depicted, mitochondric is mentioned; which is operable linked, too.

In another embodiment thereof the expression cassette does not comprise an additional transit peptide in case in Table I, application no. 15, in column 6 in the same line as SEQ ID NO. 18070 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 812, or a homolog or fragment thereof, or a homolog thereof being depicted in Table I, application no. 15, column 8, in the same line as SEQ ID NO. 812, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 812 is depicted, plastidic is mentioned, or a mitochondrial transit peptide in case in Table I, application no. 15, in column 6 in the same line as SEQ ID NO. 812 is depicted, mitochondric is mentioned; which is operable linked, too.

In another embodiment thereof the expression cassette does not comprise an additional transit peptide in case in Table I, application no. 15, in column 6 in the same line as SEQ ID NO. 812 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 19419, or a homolog or fragment thereof, or a homolog thereof being depicted in Table I, application no. 15, column 8, in the same line as SEQ ID NO. 19419, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 19419 is depicted, plastidic is mentioned, or a mitochondrial transit peptide in case in Table I, application no. 15, in column 6 in the same line as SEQ ID NO. 19419 is depicted, mitochondric is mentioned; which is operable linked, too.

In another embodiment thereof the expression cassette does not comprise an additional transit peptide in case in Table I, application no. 15, in column 6 in the same line as SEQ ID NO. 19419 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 19919, or a homolog or fragment thereof, or a homolog thereof being depicted in Table I, application no. 15, column 8, in the same line as SEQ ID NO. 19919, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 19919 is depicted, plastidic is mentioned, or a mitochondrial transit peptide in case in Table I, application no. 15, in column 6 in the same line as SEQ ID NO. 19919 is depicted, mitochondric is mentioned; which is operable linked, too.

In another embodiment thereof the expression cassette does not comprise an additional transit peptide in case in Table I, application no. 15, in column 6 in the same line as SEQ ID NO. 19919 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 133042, or a homolog or fragment thereof, or a homolog thereof being depicted in Table I, application no. 15, column 8, in the same line as SEQ ID NO. 133042, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 133042 is depicted, plastidic is mentioned, or a mitochondrial transit peptide in case in Table I, application no. 15, in column 6 in the same line as SEQ ID NO. 133042 is depicted, mitochondric is mentioned; which is operable linked, too.

In another embodiment thereof the expression cassette does not comprise an additional transit peptide in case in Table I, application no. 15, in column 6 in the same line as SEQ ID NO. 133042 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 133349, or a homolog or fragment thereof, or a homolog thereof being depicted in Table I, application no. 15, column 8, in the same line as SEQ ID NO. 133349, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 133349 is depicted, plastidic is mentioned, or a mitochondrial transit peptide in case in Table I, application no. 15, in column 6 in the same line as SEQ ID NO. 133349 is depicted, mitochondric is mentioned; which is operable linked, too.

In another embodiment thereof the expression cassette does not comprise an additional transit peptide in case in Table I, application no. 15, in column 6 in the same line as SEQ ID NO. 133349 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 120810, or a homolog or fragment thereof, or a homolog thereof being depicted in Table I, application no. 15, column 8, in the same line as SEQ ID NO. 120810, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 120810 is depicted, plastidic is mentioned, or a mitochondrial transit peptide in case in Table I, application no. 15, in column 6 in the same line as SEQ ID NO. 120810 is depicted, mitochondric is mentioned; which is operable linked, too.

In another embodiment thereof the expression cassette does not comprise an additional transit peptide in case in Table I, application no. 15, in column 6 in the same line as SEQ ID NO. 120810 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 21497, or a homolog or fragment thereof, or a homolog thereof being depicted in Table I, application no. 15, column 8, in the same line as SEQ ID NO. 21497, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 21497 is depicted, plastidic is mentioned, or a mitochondrial transit peptide in case in Table I, application no. 15, in column 6 in the same line as SEQ ID NO. 21497 is depicted, mitochondric is mentioned; which is operable linked, too.

In another embodiment thereof the expression cassette does not comprise an additional transit peptide in case in Table I, application no. 15, in column 6 in the same line as SEQ ID NO. 21497 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 22015, or a homolog or fragment thereof, or a homolog thereof being depicted in Table I, application no. 15, column 8, in the same line as SEQ ID NO. 22015, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 22015 is depicted, plastidic is mentioned, or a mitochondrial transit peptide in case in Table I, application no. 15, in column 6 in the same line as SEQ ID NO. 22015 is depicted, mitochondric is mentioned; which is operable linked, too.

In another embodiment thereof the expression cassette does not comprise an additional transit peptide in case in Table I, application no. 15, in column 6 in the same line as SEQ ID NO. 22015 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 22249, or a homolog or fragment thereof, or a homolog thereof being depicted in Table I, application no. 15, column 8, in the same line as SEQ ID NO. 22249, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 22249 is depicted, plastidic is mentioned, or a mitochondrial transit peptide in case in Table I, application no. 15, in column 6 in the same line as SEQ ID NO. 22249 is depicted, mitochondric is mentioned; which is operable linked, too.

In another embodiment thereof the expression cassette does not comprise an additional transit peptide in case in Table I, application no. 15, in column 6 in the same line as SEQ ID NO. 22249 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 130491, or a homolog or fragment thereof, or a homolog thereof being depicted in Table I, application no. 15, column 8, in the same line as SEQ ID NO. 130491, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 130491 is depicted, plastidic is mentioned, or a mitochondrial transit peptide in case in Table I, application no. 15, in column 6 in the same line as SEQ ID NO. 130491 is depicted, mitochondric is mentioned; which is operable linked, too.

In another embodiment thereof the expression cassette does not comprise an additional transit peptide in case in Table I, application no. 15, in column 6 in the same line as SEQ ID NO. 130491 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 2367, or a homolog or fragment thereof, or a homolog thereof being depicted in Table I, application no. 15, column 8, in the same line as SEQ ID NO. 2367, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 2367 is depicted, plastidic is mentioned, or a mitochondrial transit peptide in case in Table I, application no. 15, in column 6 in the same line as SEQ ID NO. 2367 is depicted, mitochondric is mentioned; which is operable linked, too.

In another embodiment thereof the expression cassette does not comprise an additional transit peptide in case in Table I, application no. 15, in column 6 in the same line as SEQ ID NO. 2367 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 2573, or a homolog or fragment thereof, or a homolog thereof being depicted in Table I, application no. 15, column 8, in the same line as SEQ ID NO. 2573, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 2573 is depicted, plastidic is mentioned, or a mitochondrial transit peptide in case in Table I, application no. 15, in column 6 in the same line as SEQ ID NO. 2573 is depicted, mitochondric is mentioned; which is operable linked, too.

In another embodiment thereof the expression cassette does not comprise an additional transit peptide in case in Table I, application no. 15, in column 6 in the same line as SEQ ID NO. 2573 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 2935, or a homolog or fragment thereof, or a homolog thereof being depicted in Table I, application no. 15, column 8, in the same line as SEQ ID NO. 2935, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 2935 is depicted, plastidic is mentioned, or a mitochondrial transit peptide in case in Table I, application no. 15, in column 6 in the same line as SEQ ID NO. 2935 is depicted, mitochondric is mentioned; which is operable linked, too.

In another embodiment thereof the expression cassette does not comprise an additional transit peptide in case in Table I, application no. 15, in column 6 in the same line as SEQ ID NO. 2935 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 133541, or a homolog or fragment thereof, or a homolog thereof being depicted in Table I, application no. 15, column 8, in the same line as SEQ ID NO. 133541, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 133541 is depicted, plastidic is mentioned, or a mitochondrial transit peptide in case in Table I, application no. 15, in column 6 in the same line as SEQ ID NO. 133541 is depicted, mitochondric is mentioned; which is operable linked, too.

In another embodiment thereof the expression cassette does not comprise an additional transit peptide in case in Table I, application no. 15, in column 6 in the same line as SEQ ID NO. 133541 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 23482, or a homolog or fragment thereof, or a homolog thereof being depicted in Table I, application no. 15, column 8, in the same line as SEQ ID NO. 23482, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 23482 is depicted, plastidic is mentioned, or a mitochondrial transit peptide in case in Table I, application no. 15, in column 6 in the same line as SEQ ID NO. 23482 is depicted, mitochondric is mentioned; which is operable linked, too.

In another embodiment thereof the expression cassette does not comprise an additional transit peptide in case in Table I, application no. 15, in column 6 in the same line as SEQ ID NO. 23482 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 3279, or a homolog or fragment thereof, or a homolog thereof being depicted in Table I, application no. 15, column 8, in the same line as SEQ ID NO. 3279, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 3279 is depicted, plastidic is mentioned, or a mitochondrial transit peptide in case in Table I, application no. 15, in column 6 in the same line as SEQ ID NO. 3279 is depicted, mitochondric is mentioned; which is operable linked, too.

In another embodiment thereof the expression cassette does not comprise an additional transit peptide in case in Table I, application no. 15, in column 6 in the same line as SEQ ID NO. 3279 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 23844, or a homolog or fragment thereof, or a homolog thereof being depicted in Table I, application no. 15, column 8, in the same line as SEQ ID NO. 23844, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 23844 is depicted, plastidic is mentioned, or a mitochondrial transit peptide in case in Table I, application no. 15, in column 6 in the same line as SEQ ID NO. 23844 is depicted, mitochondric is mentioned; which is operable linked, too.

In another embodiment thereof the expression cassette does not comprise an additional transit peptide in case in Table I, application no. 15, in column 6 in the same line as SEQ ID NO. 23844 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 3654, or a homolog or fragment thereof, or a homolog thereof being depicted in Table I, application no. 15, column 8, in the same line as SEQ ID NO. 3654, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 3654 is depicted, plastidic is mentioned, or a mitochondrial transit peptide in case in Table I, application no. 15, in column 6 in the same line as SEQ ID NO. 3654 is depicted, mitochondric is mentioned; which is operable linked, too.

In another embodiment thereof the expression cassette does not comprise an additional transit peptide in case in Table I, application no. 15, in column 6 in the same line as SEQ ID NO. 3654 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68849, or a homolog or fragment thereof, or a homolog thereof being depicted in Table I, application no. 15, column 8, in the same line as SEQ ID NO. 68849, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 68849 is depicted, plastidic is mentioned, or a mitochondrial transit peptide in case in Table I, application no. 15, in column 6 in the same line as SEQ ID NO. 68849 is depicted, mitochondric is mentioned; which is operable linked, too.

In another embodiment thereof the expression cassette does not comprise an additional transit peptide in case in Table I, application no. 15, in column 6 in the same line as SEQ ID NO. 68849 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4102, or a homolog or fragment thereof, or a homolog thereof being depicted in Table I, application no. 15, column 8, in the same line as SEQ ID NO. 4102, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 4102 is depicted, plastidic is mentioned, or a mitochondrial transit peptide in case in Table I, application no. 15, in column 6 in the same line as SEQ ID NO. 4102 is depicted, mitochondric is mentioned; which is operable linked, too.

In another embodiment thereof the expression cassette does not comprise an additional transit peptide in case in Table I, application no. 15, in column 6 in the same line as SEQ ID NO. 4102 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4348, or a homolog or fragment thereof, or a homolog thereof being depicted in Table I, application no. 15, column 8, in the same line as SEQ ID NO. 4348, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 4348 is depicted, plastidic is mentioned, or a mitochondrial transit peptide in case in Table I, application no. 15, in column 6 in the same line as SEQ ID NO. 4348 is depicted, mitochondric is mentioned; which is operable linked, too.

In another embodiment thereof the expression cassette does not comprise an additional transit peptide in case in Table I, application no. 15, in column 6 in the same line as SEQ ID NO. 4348 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 24438, or a homolog or fragment thereof, or a homolog thereof being depicted in Table I, application no. 15, column 8, in the same line as SEQ ID NO. 24438, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 24438 is depicted, plastidic is mentioned, or a mitochondrial transit peptide in case in Table I, application no. 15, in column 6 in the same line as SEQ ID NO. 24438 is depicted, mitochondric is mentioned; which is operable linked, too.

In another embodiment thereof the expression cassette does not comprise an additional transit peptide in case in Table I, application no. 15, in column 6 in the same line as SEQ ID NO. 24438 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 24492, or a homolog or fragment thereof, or a homolog thereof being depicted in Table I, application no. 15, column 8, in the same line as SEQ ID NO. 24492, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 24492 is depicted, plastidic is mentioned, or a mitochondrial transit peptide in case in Table I, application no. 15, in column 6 in the same line as SEQ ID NO. 24492 is depicted, mitochondric is mentioned; which is operable linked, too.

In another embodiment thereof the expression cassette does not comprise an additional transit peptide in case in Table I, application no. 15, in column 6 in the same line as SEQ ID NO. 24492 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4904, or a homolog or fragment thereof, or a homolog thereof being depicted in Table I, application no. 15, column 8, in the same line as SEQ ID NO. 4904, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 4904 is depicted, plastidic is mentioned, or a mitochondrial transit peptide in case in Table I, application no. 15, in column 6 in the same line as SEQ ID NO. 4904 is depicted, mitochondric is mentioned; which is operable linked, too.

In another embodiment thereof the expression cassette does not comprise an additional transit peptide in case in Table I, application no. 15, in column 6 in the same line as SEQ ID NO. 4904 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 25344, or a homolog or fragment thereof, or a homolog thereof being depicted in Table I, application no. 15, column 8, in the same line as SEQ ID NO. 25344, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 25344 is depicted, plastidic is mentioned, or a mitochondrial transit peptide in case in Table I, application no. 15, in column 6 in the same line as SEQ ID NO. 25344 is depicted, mitochondric is mentioned; which is operable linked, too.

In another embodiment thereof the expression cassette does not comprise an additional transit peptide in case in Table I, application no. 15, in column 6 in the same line as SEQ ID NO. 25344 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 25428, or a homolog or fragment thereof, or a homolog thereof being depicted in Table I, application no. 15, column 8, in the same line as SEQ ID NO. 25428, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 25428 is depicted, plastidic is mentioned, or a mitochondrial transit peptide in case in Table I, application no. 15, in column 6 in the same line as SEQ ID NO. 25428 is depicted, mitochondric is mentioned; which is operable linked, too.

In another embodiment thereof the expression cassette does not comprise an additional transit peptide in case in Table I, application no. 15, in column 6 in the same line as SEQ ID NO. 25428 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 25498, or a homolog or fragment thereof, or a homolog thereof being depicted in Table I, application no. 15, column 8, in the same line as SEQ ID NO. 25498, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 25498 is depicted, plastidic is mentioned, or a mitochondrial transit peptide in case in Table I, application no. 15, in column 6 in the same line as SEQ ID NO. 25498 is depicted, mitochondric is mentioned; which is operable linked, too.

In another embodiment thereof the expression cassette does not comprise an additional transit peptide in case in Table I, application no. 15, in column 6 in the same line as SEQ ID NO. 25498 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 70038, or a homolog or fragment thereof, or a homolog thereof being depicted in Table I, application no. 15, column 8, in the same line as SEQ ID NO. 70038, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 70038 is depicted, plastidic is mentioned, or a mitochondrial transit peptide in case in Table I, application no. 15, in column 6 in the same line as SEQ ID NO. 70038 is depicted, mitochondric is mentioned; which is operable linked, too.

In another embodiment thereof the expression cassette does not comprise an additional transit peptide in case in Table I, application no. 15, in column 6 in the same line as SEQ ID NO. 70038 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 5493, or a homolog or fragment thereof, or a homolog thereof being depicted in Table I, application no. 15, column 8, in the same line as SEQ ID NO. 5493, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 5493 is depicted, plastidic is mentioned, or a mitochondrial transit peptide in case in Table I, application no. 15, in column 6 in the same line as SEQ ID NO. 5493 is depicted, mitochondric is mentioned; which is operable linked, too.

In another embodiment thereof the expression cassette does not comprise an additional transit peptide in case in Table I, application no. 15, in column 6 in the same line as SEQ ID NO. 5493 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 70583, or a homolog or fragment thereof, or a homolog thereof being depicted in Table I, application no. 15, column 8, in the same line as SEQ ID NO. 70583, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 70583 is depicted, plastidic is mentioned, or a mitochondrial transit peptide in case in Table I, application no. 15, in column 6 in the same line as SEQ ID NO. 70583 is depicted, mitochondric is mentioned; which is operable linked, too.

In another embodiment thereof the expression cassette does not comprise an additional transit peptide in case in Table I, application no. 15, in column 6 in the same line as SEQ ID NO. 70583 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 70715, or a homolog or fragment thereof, or a homolog thereof being depicted in Table I, application no. 15, column 8, in the same line as SEQ ID NO. 70715, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 70715 is depicted, plastidic is mentioned, or a mitochondrial transit peptide in case in Table I, application no. 15, in column 6 in the same line as SEQ ID NO. 70715 is depicted, mitochondric is mentioned; which is operable linked, too.

In another embodiment thereof the expression cassette does not comprise an additional transit peptide in case in Table I, application no. 15, in column 6 in the same line as SEQ ID NO. 70715 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 25676, or a homolog or fragment thereof, or a homolog thereof being depicted in Table I, application no. 15, column 8, in the same line as SEQ ID NO. 25676, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 25676 is depicted, plastidic is mentioned, or a mitochondrial transit peptide in case in Table I, application no. 15, in column 6 in the same line as SEQ ID NO. 25676 is depicted, mitochondric is mentioned; which is operable linked, too.

In another embodiment thereof the expression cassette does not comprise an additional transit peptide in case in Table I, application no. 15, in column 6 in the same line as SEQ ID NO. 25676 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 25780, or a homolog or fragment thereof, or a homolog thereof being depicted in Table I, application no. 15, column 8, in the same line as SEQ ID NO. 25780, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 25780 is depicted, plastidic is mentioned, or a mitochondrial transit peptide in case in Table I, application no. 15, in column 6 in the same line as SEQ ID NO. 25780 is depicted, mitochondric is mentioned; which is operable linked, too.

In another embodiment thereof the expression cassette does not comprise an additional transit peptide in case in Table I, application no. 15, in column 6 in the same line as SEQ ID NO. 25780 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 26196, or a homolog or fragment thereof, or a homolog thereof being depicted in Table I, application no. 15, column 8, in the same line as SEQ ID NO. 26196, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 26196 is depicted, plastidic is mentioned, or a mitochondrial transit peptide in case in Table I, application no. 15, in column 6 in the same line as SEQ ID NO. 26196 is depicted, mitochondric is mentioned; which is operable linked, too.

In another embodiment thereof the expression cassette does not comprise an additional transit peptide in case in Table I, application no. 15, in column 6 in the same line as SEQ ID NO. 26196 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 133719, or a homolog or fragment thereof, or a homolog thereof being depicted in Table I, application no. 15, column 8, in the same line as SEQ ID NO. 133719, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 133719 is depicted, plastidic is mentioned, or a mitochondrial transit peptide in case in Table I, application no. 15, in column 6 in the same line as SEQ ID NO. 133719 is depicted, mitochondric is mentioned; which is operable linked, too.

In another embodiment thereof the expression cassette does not comprise an additional transit peptide in case in Table I, application no. 15, in column 6 in the same line as SEQ ID NO. 133719 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 26434, or a homolog or fragment thereof, or a homolog thereof being depicted in Table I, application no. 15, column 8, in the same line as SEQ ID NO. 26434, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 26434 is depicted, plastidic is mentioned, or a mitochondrial transit peptide in case in Table I, application no. 15, in column 6 in the same line as SEQ ID NO. 26434 is depicted, mitochondric is mentioned; which is operable linked, too.

In another embodiment thereof the expression cassette does not comprise an additional transit peptide in case in Table I, application no. 15, in column 6 in the same line as SEQ ID NO. 26434 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 114198, or a homolog or fragment thereof, or a homolog thereof being depicted in Table I, application no. 15, column 8, in the same line as SEQ ID NO. 114198, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 114198 is depicted, plastidic is mentioned, or a mitochondrial transit peptide in case in Table I, application no. 15, in column 6 in the same line as SEQ ID NO. 114198 is depicted, mitochondric is mentioned; which is operable linked, too.

In another embodiment thereof the expression cassette does not comprise an additional transit peptide in case in Table I, application no. 15, in column 6 in the same line as SEQ ID NO. 114198 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6040, or a homolog or fragment thereof, or a homolog thereof being depicted in Table I, application no. 15, column 8, in the same line as SEQ ID NO. 6040, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 6040 is depicted, plastidic is mentioned, or a mitochondrial transit peptide in case in Table I, application no. 15, in column 6 in the same line as SEQ ID NO. 6040 is depicted, mitochondric is mentioned; which is operable linked, too.

In another embodiment thereof the expression cassette does not comprise an additional transit peptide in case in Table I, application no. 15, in column 6 in the same line as SEQ ID NO. 6040 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 71325, or a homolog or fragment thereof, or a homolog thereof being depicted in Table I, application no. 15, column 8, in the same line as SEQ ID NO. 71325, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 71325 is depicted, plastidic is mentioned, or a mitochondrial transit peptide in case in Table I, application no. 15, in column 6 in the same line as SEQ ID NO. 71325 is depicted, mitochondric is mentioned; which is operable linked, too.

In another embodiment thereof the expression cassette does not comprise an additional transit peptide in case in Table I, application no. 15, in column 6 in the same line as SEQ ID NO. 71325 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 71763, or a homolog or fragment thereof, or a homolog thereof being depicted in Table I, application no. 15, column 8, in the same line as SEQ ID NO. 71763, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 71763 is depicted, plastidic is mentioned, or a mitochondrial transit peptide in case in Table I, application no. 15, in column 6 in the same line as SEQ ID NO. 71763 is depicted, mitochondric is mentioned; which is operable linked, too.

In another embodiment thereof the expression cassette does not comprise an additional transit peptide in case in Table I, application no. 15, in column 6 in the same line as SEQ ID NO. 71763 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 28040, or a homolog or fragment thereof, or a homolog thereof being depicted in Table I, application no. 15, column 8, in the same line as SEQ ID NO. 28040, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 28040 is depicted, plastidic is mentioned, or a mitochondrial transit peptide in case in Table I, application no. 15, in column 6 in the same line as SEQ ID NO. 28040 is depicted, mitochondric is mentioned; which is operable linked, too.

In another embodiment thereof the expression cassette does not comprise an additional transit peptide in case in Table I, application no. 15, in column 6 in the same line as SEQ ID NO. 28040 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6075, or a homolog or fragment thereof, or a homolog thereof being depicted in Table I, application no. 15, column 8, in the same line as SEQ ID NO. 6075, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 6075 is depicted, plastidic is mentioned, or a mitochondrial transit peptide in case in Table I, application no. 15, in column 6 in the same line as SEQ ID NO. 6075 is depicted, mitochondric is mentioned; which is operable linked, too.

In another embodiment thereof the expression cassette does not comprise an additional transit peptide in case in Table I, application no. 15, in column 6 in the same line as SEQ ID NO. 6075 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 29246, or a homolog or fragment thereof, or a homolog thereof being depicted in Table I, application no. 15, column 8, in the same line as SEQ ID NO. 29246, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 29246 is depicted, plastidic is mentioned, or a mitochondrial transit peptide in case in Table I, application no. 15, in column 6 in the same line as SEQ ID NO. 29246 is depicted, mitochondric is mentioned; which is operable linked, too.

In another embodiment thereof the expression cassette does not comprise an additional transit peptide in case in Table I, application no. 15, in column 6 in the same line as SEQ ID NO. 29246 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 29500, or a homolog or fragment thereof, or a homolog thereof being depicted in Table I, application no. 15, column 8, in the same line as SEQ ID NO. 29500, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 29500 is depicted, plastidic is mentioned, or a mitochondrial transit peptide in case in Table I, application no. 15, in column 6 in the same line as SEQ ID NO. 29500 is depicted, mitochondric is mentioned; which is operable linked, too.

In another embodiment thereof the expression cassette does not comprise an additional transit peptide in case in Table I, application no. 15, in column 6 in the same line as SEQ ID NO. 29500 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 31026, or a homolog or fragment thereof, or a homolog thereof being depicted in Table I, application no. 15, column 8, in the same line as SEQ ID NO. 31026, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 31026 is depicted, plastidic is mentioned, or a mitochondrial transit peptide in case in Table I, application no. 15, in column 6 in the same line as SEQ ID NO. 31026 is depicted, mitochondric is mentioned; which is operable linked, too.

In another embodiment thereof the expression cassette does not comprise an additional transit peptide in case in Table I, application no. 15, in column 6 in the same line as SEQ ID NO. 31026 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 114231, or a homolog or fragment thereof, or a homolog thereof being depicted in Table I, application no. 15, column 8, in the same line as SEQ ID NO. 114231, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 114231 is depicted, plastidic is mentioned, or a mitochondrial transit peptide in case in Table I, application no. 15, in column 6 in the same line as SEQ ID NO. 114231 is depicted, mitochondric is mentioned; which is operable linked, too.

In another embodiment thereof the expression cassette does not comprise an additional transit peptide in case in Table I, application no. 15, in column 6 in the same line as SEQ ID NO. 114231 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 31717, or a homolog or fragment thereof, or a homolog thereof being depicted in Table I, application no. 15, column 8, in the same line as SEQ ID NO. 31717, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 31717 is depicted, plastidic is mentioned, or a mitochondrial transit peptide in case in Table I, application no. 15, in column 6 in the same line as SEQ ID NO. 31717 is depicted, mitochondric is mentioned; which is operable linked, too.

In another embodiment thereof the expression cassette does not comprise an additional transit peptide in case in Table I, application no. 15, in column 6 in the same line as SEQ ID NO. 31717 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 72935, or a homolog or fragment thereof, or a homolog thereof being depicted in Table I, application no. 15, column 8, in the same line as SEQ ID NO. 72935, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 72935 is depicted, plastidic is mentioned, or a mitochondrial transit peptide in case in Table I, application no. 15, in column 6 in the same line as SEQ ID NO. 72935 is depicted, mitochondric is mentioned; which is operable linked, too.

In another embodiment thereof the expression cassette does not comprise an additional transit peptide in case in Table I, application no. 15, in column 6 in the same line as SEQ ID NO. 72935 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 31926, or a homolog or fragment thereof, or a homolog thereof being depicted in Table I, application no. 15, column 8, in the same line as SEQ ID NO. 31926, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 31926 is depicted, plastidic is mentioned, or a mitochondrial transit peptide in case in Table I, application no. 15, in column 6 in the same line as SEQ ID NO. 31926 is depicted, mitochondric is mentioned; which is operable linked, too.

In another embodiment thereof the expression cassette does not comprise an additional transit peptide in case in Table I, application no. 15, in column 6 in the same line as SEQ ID NO. 31926 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 134171, or a homolog or fragment thereof, or a homolog thereof being depicted in Table I, application no. 15, column 8, in the same line as SEQ ID NO. 134171, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 134171 is depicted, plastidic is mentioned, or a mitochondrial transit peptide in case in Table I, application no. 15, in column 6 in the same line as SEQ ID NO. 134171 is depicted, mitochondric is mentioned; which is operable linked, too.

In another embodiment thereof the expression cassette does not comprise an additional transit peptide in case in Table I, application no. 15, in column 6 in the same line as SEQ ID NO. 134171 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 73719, or a homolog or fragment thereof, or a homolog thereof being depicted in Table I, application no. 15, column 8, in the same line as SEQ ID NO. 73719, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 73719 is depicted, plastidic is mentioned, or a mitochondrial transit peptide in case in Table I, application no. 15, in column 6 in the same line as SEQ ID NO. 73719 is depicted, mitochondric is mentioned; which is operable linked, too.

In another embodiment thereof the expression cassette does not comprise an additional transit peptide in case in Table I, application no. 15, in column 6 in the same line as SEQ ID NO. 73719 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 74452, or a homolog or fragment thereof, or a homolog thereof being depicted in Table I, application no. 15, column 8, in the same line as SEQ ID NO. 74452, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 74452 is depicted, plastidic is mentioned, or a mitochondrial transit peptide in case in Table I, application no. 15, in column 6 in the same line as SEQ ID NO. 74452 is depicted, mitochondric is mentioned; which is operable linked, too.

In another embodiment thereof the expression cassette does not comprise an additional transit peptide in case in Table I, application no. 15, in column 6 in the same line as SEQ ID NO. 74452 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6510, or a homolog or fragment thereof, or a homolog thereof being depicted in Table I, application no. 15, column 8, in the same line as SEQ ID NO. 6510, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 6510 is depicted, plastidic is mentioned, or a mitochondrial transit peptide in case in Table I, application no. 15, in column 6 in the same line as SEQ ID NO. 6510 is depicted, mitochondric is mentioned; which is operable linked, too.

In another embodiment thereof the expression cassette does not comprise an additional transit peptide in case in Table I, application no. 15, in column 6 in the same line as SEQ ID NO. 6510 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 32037, or a homolog or fragment thereof, or a homolog thereof being depicted in Table I, application no. 15, column 8, in the same line as SEQ ID NO. 32037, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 32037 is depicted, plastidic is mentioned, or a mitochondrial transit peptide in case in Table I, application no. 15, in column 6 in the same line as SEQ ID NO. 32037 is depicted, mitochondric is mentioned; which is operable linked, too.

In another embodiment thereof the expression cassette does not comprise an additional transit peptide in case in Table I, application no. 15, in column 6 in the same line as SEQ ID NO. 32037 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 74729, or a homolog or fragment thereof, or a homolog thereof being depicted in Table I, application no. 15, column 8, in the same line as SEQ ID NO. 74729, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 74729 is depicted, plastidic is mentioned, or a mitochondrial transit peptide in case in Table I, application no. 15, in column 6 in the same line as SEQ ID NO. 74729 is depicted, mitochondric is mentioned; which is operable linked, too.

In another embodiment thereof the expression cassette does not comprise an additional transit peptide in case in Table I, application no. 15, in column 6 in the same line as SEQ ID NO. 74729 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 34301, or a homolog or fragment thereof, or a homolog thereof being depicted in Table I, application no. 15, column 8, in the same line as SEQ ID NO. 34301, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 34301 is depicted, plastidic is mentioned, or a mitochondrial transit peptide in case in Table I, application no. 15, in column 6 in the same line as SEQ ID NO. 34301 is depicted, mitochondric is mentioned; which is operable linked, too.

In another embodiment thereof the expression cassette does not comprise an additional transit peptide in case in Table I, application no. 15, in column 6 in the same line as SEQ ID NO. 34301 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 34602, or a homolog or fragment thereof, or a homolog thereof being depicted in Table I, application no. 15, column 8, in the same line as SEQ ID NO. 34602, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 34602 is depicted, plastidic is mentioned, or a mitochondrial transit peptide in case in Table I, application no. 15, in column 6 in the same line as SEQ ID NO. 34602 is depicted, mitochondric is mentioned; which is operable linked, too.

In another embodiment thereof the expression cassette does not comprise an additional transit peptide in case in Table I, application no. 15, in column 6 in the same line as SEQ ID NO. 34602 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 136120, or a homolog or fragment thereof, or a homolog thereof being depicted in Table I, application no. 15, column 8, in the same line as SEQ ID NO. 136120, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 136120 is depicted, plastidic is mentioned, or a mitochondrial transit peptide in case in Table I, application no. 15, in column 6 in the same line as SEQ ID NO. 136120 is depicted, mitochondric is mentioned; which is operable linked, too.

In another embodiment thereof the expression cassette does not comprise an additional transit peptide in case in Table I, application no. 15, in column 6 in the same line as SEQ ID NO. 136120 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 34889, or a homolog or fragment thereof, or a homolog thereof being depicted in Table I, application no. 15, column 8, in the same line as SEQ ID NO. 34889, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 34889 is depicted, plastidic is mentioned, or a mitochondrial transit peptide in case in Table I, application no. 15, in column 6 in the same line as SEQ ID NO. 34889 is depicted, mitochondric is mentioned; which is operable linked, too.

In another embodiment thereof the expression cassette does not comprise an additional transit peptide in case in Table I, application no. 15, in column 6 in the same line as SEQ ID NO. 34889 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 134299, or a homolog or fragment thereof, or a homolog thereof being depicted in Table I, application no. 15, column 8, in the same line as SEQ ID NO. 134299, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 134299 is depicted, plastidic is mentioned, or a mitochondrial transit peptide in case in Table I, application no. 15, in column 6 in the same line as SEQ ID NO. 134299 is depicted, mitochondric is mentioned; which is operable linked, too.

In another embodiment thereof the expression cassette does not comprise an additional transit peptide in case in Table I, application no. 15, in column 6 in the same line as SEQ ID NO. 134299 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 35482, or a homolog or fragment thereof, or a homolog thereof being depicted in Table I, application no. 15, column 8, in the same line as SEQ ID NO. 35482, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 35482 is depicted, plastidic is mentioned, or a mitochondrial transit peptide in case in Table I, application no. 15, in column 6 in the same line as SEQ ID NO. 35482 is depicted, mitochondric is mentioned; which is operable linked, too.

In another embodiment thereof the expression cassette does not comprise an additional transit peptide in case in Table I, application no. 15, in column 6 in the same line as SEQ ID NO. 35482 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 35590, or a homolog or fragment thereof, or a homolog thereof being depicted in Table I, application no. 15, column 8, in the same line as SEQ ID NO. 35590, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 35590 is depicted, plastidic is mentioned, or a mitochondrial transit peptide in case in Table I, application no. 15, in column 6 in the same line as SEQ ID NO. 35590 is depicted, mitochondric is mentioned; which is operable linked, too.

In another embodiment thereof the expression cassette does not comprise an additional transit peptide in case in Table I, application no. 15, in column 6 in the same line as SEQ ID NO. 35590 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 121111, or a homolog or fragment thereof, or a homolog thereof being depicted in Table I, application no. 15, column 8, in the same line as SEQ ID NO. 121111, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 121111 is depicted, plastidic is mentioned, or a mitochondrial transit peptide in case in Table I, application no. 15, in column 6 in the same line as SEQ ID NO. 121111 is depicted, mitochondric is mentioned; which is operable linked, too.

In another embodiment thereof the expression cassette does not comprise an additional transit peptide in case in Table I, application no. 15, in column 6 in the same line as SEQ ID NO. 121111 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7333, or a homolog or fragment thereof, or a homolog thereof being depicted in Table I, application no. 15, column 8, in the same line as SEQ ID NO. 7333, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 7333 is depicted, plastidic is mentioned, or a mitochondrial transit peptide in case in Table I, application no. 15, in column 6 in the same line as SEQ ID NO. 7333 is depicted, mitochondric is mentioned; which is operable linked, too.

In another embodiment thereof the expression cassette does not comprise an additional transit peptide in case in Table I, application no. 15, in column 6 in the same line as SEQ ID NO. 7333 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7686, or a homolog or fragment thereof, or a homolog thereof being depicted in Table I, application no. 15, column 8, in the same line as SEQ ID NO. 7686, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 7686 is depicted, plastidic is mentioned, or a mitochondrial transit peptide in case in Table I, application no. 15, in column 6 in the same line as SEQ ID NO. 7686 is depicted, mitochondric is mentioned; which is operable linked, too.

In another embodiment thereof the expression cassette does not comprise an additional transit peptide in case in Table I, application no. 15, in column 6 in the same line as SEQ ID NO. 7686 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 134402, or a homolog or fragment thereof, or a homolog thereof being depicted in Table I, application no. 15, column 8, in the same line as SEQ ID NO. 134402, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 134402 is depicted, plastidic is mentioned, or a mitochondrial transit peptide in case in Table I, application no. 15, in column 6 in the same line as SEQ ID NO. 134402 is depicted, mitochondric is mentioned; which is operable linked, too.

In another embodiment thereof the expression cassette does not comprise an additional transit peptide in case in Table I, application no. 15, in column 6 in the same line as SEQ ID NO. 134402 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 35967, or a homolog or fragment thereof, or a homolog thereof being depicted in Table I, application no. 15, column 8, in the same line as SEQ ID NO. 35967, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 35967 is depicted, plastidic is mentioned, or a mitochondrial transit peptide in case in Table I, application no. 15, in column 6 in the same line as SEQ ID NO. 35967 is depicted, mitochondric is mentioned; which is operable linked, too.

In another embodiment thereof the expression cassette does not comprise an additional transit peptide in case in Table I, application no. 15, in column 6 in the same line as SEQ ID NO. 35967 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 36114, or a homolog or fragment thereof, or a homolog thereof being depicted in Table I, application no. 15, column 8, in the same line as SEQ ID NO. 36114, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 36114 is depicted, plastidic is mentioned, or a mitochondrial transit peptide in case in Table I, application no. 15, in column 6 in the same line as SEQ ID NO. 36114 is depicted, mitochondric is mentioned; which is operable linked, too.

In another embodiment thereof the expression cassette does not comprise an additional transit peptide in case in Table I, application no. 15, in column 6 in the same line as SEQ ID NO. 36114 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 75807, or a homolog or fragment thereof, or a homolog thereof being depicted in Table I, application no. 15, column 8, in the same line as SEQ ID NO. 75807, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 75807 is depicted, plastidic is mentioned, or a mitochondrial transit peptide in case in Table I, application no. 15, in column 6 in the same line as SEQ ID NO. 75807 is depicted, mitochondric is mentioned; which is operable linked, too.

In another embodiment thereof the expression cassette does not comprise an additional transit peptide in case in Table I, application no. 15, in column 6 in the same line as SEQ ID NO. 75807 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 36489, or a homolog or fragment thereof, or a homolog thereof being depicted in Table I, application no. 15, column 8, in the same line as SEQ ID NO. 36489, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 36489 is depicted, plastidic is mentioned, or a mitochondrial transit peptide in case in Table I, application no. 15, in column 6 in the same line as SEQ ID NO. 36489 is depicted, mitochondric is mentioned; which is operable linked, too.

In another embodiment thereof the expression cassette does not comprise an additional transit peptide in case in Table I, application no. 15, in column 6 in the same line as SEQ ID NO. 36489 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 36670, or a homolog or fragment thereof, or a homolog thereof being depicted in Table I, application no. 15, column 8, in the same line as SEQ ID NO. 36670, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 36670 is depicted, plastidic is mentioned, or a mitochondrial transit peptide in case in Table I, application no. 15, in column 6 in the same line as SEQ ID NO. 36670 is depicted, mitochondric is mentioned; which is operable linked, too.

In another embodiment thereof the expression cassette does not comprise an additional transit peptide in case in Table I, application no. 15, in column 6 in the same line as SEQ ID NO. 36670 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7941, or a homolog or fragment thereof, or a homolog thereof being depicted in Table I, application no. 15, column 8, in the same line as SEQ ID NO. 7941, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 7941 is depicted, plastidic is mentioned, or a mitochondrial transit peptide in case in Table I, application no. 15, in column 6 in the same line as SEQ ID NO. 7941 is depicted, mitochondric is mentioned; which is operable linked, too.

In another embodiment thereof the expression cassette does not comprise an additional transit peptide in case in Table I, application no. 15, in column 6 in the same line as SEQ ID NO. 7941 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 37483, or a homolog or fragment thereof, or a homolog thereof being depicted in Table I, application no. 15, column 8, in the same line as SEQ ID NO. 37483, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 37483 is depicted, plastidic is mentioned, or a mitochondrial transit peptide in case in Table I, application no. 15, in column 6 in the same line as SEQ ID NO. 37483 is depicted, mitochondric is mentioned; which is operable linked, too.

In another embodiment thereof the expression cassette does not comprise an additional transit peptide in case in Table I, application no. 15, in column 6 in the same line as SEQ ID NO. 37483 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 134655, or a homolog or fragment thereof, or a homolog thereof being depicted in Table I, application no. 15, column 8, in the same line as SEQ ID NO. 134655, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 134655 is depicted, plastidic is mentioned, or a mitochondrial transit peptide in case in Table I, application no. 15, in column 6 in the same line as SEQ ID NO. 134655 is depicted, mitochondric is mentioned; which is operable linked, too.

In another embodiment thereof the expression cassette does not comprise an additional transit peptide in case in Table I, application no. 15, in column 6 in the same line as SEQ ID NO. 134655 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 134697, or a homolog or fragment thereof, or a homolog thereof being depicted in Table I, application no. 15, column 8, in the same line as SEQ ID NO. 134697, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 134697 is depicted, plastidic is mentioned, or a mitochondrial transit peptide in case in Table I, application no. 15, in column 6 in the same line as SEQ ID NO. 134697 is depicted, mitochondric is mentioned; which is operable linked, too.

In another embodiment thereof the expression cassette does not comprise an additional transit peptide in case in Table I, application no. 15, in column 6 in the same line as SEQ ID NO. 134697 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 134756, or a homolog or fragment thereof, or a homolog thereof being depicted in Table I, application no. 15, column 8, in the same line as SEQ ID NO. 134756, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 134756 is depicted, plastidic is mentioned, or a mitochondrial transit peptide in case in Table I, application no. 15, in column 6 in the same line as SEQ ID NO. 134756 is depicted, mitochondric is mentioned; which is operable linked, too.

In another embodiment thereof the expression cassette does not comprise an additional transit peptide in case in Table I, application no. 15, in column 6 in the same line as SEQ ID NO. 134756 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38266, or a homolog or fragment thereof, or a homolog thereof being depicted in Table I, application no. 15, column 8, in the same line as SEQ ID NO. 38266, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 38266 is depicted, plastidic is mentioned, or a mitochondrial transit peptide in case in Table I, application no. 15, in column 6 in the same line as SEQ ID NO. 38266 is depicted, mitochondric is mentioned; which is operable linked, too.

In another embodiment thereof the expression cassette does not comprise an additional transit peptide in case in Table I, application no. 15, in column 6 in the same line as SEQ ID NO. 38266 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38289, or a homolog or fragment thereof, or a homolog thereof being depicted in Table I, application no. 15, column 8, in the same line as SEQ ID NO. 38289, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 38289 is depicted, plastidic is mentioned, or a mitochondrial transit peptide in case in Table I, application no. 15, in column 6 in the same line as SEQ ID NO. 38289 is depicted, mitochondric is mentioned; which is operable linked, too.

In another embodiment thereof the expression cassette does not comprise an additional transit peptide in case in Table I, application no. 15, in column 6 in the same line as SEQ ID NO. 38289 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7947, or a homolog or fragment thereof, or a homolog thereof being depicted in Table I, application no. 15, column 8, in the same line as SEQ ID NO. 7947, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 7947 is depicted, plastidic is mentioned, or a mitochondrial transit peptide in case in Table I, application no. 15, in column 6 in the same line as SEQ ID NO. 7947 is depicted, mitochondric is mentioned; which is operable linked, too.

In another embodiment thereof the expression cassette does not comprise an additional transit peptide in case in Table I, application no. 15, in column 6 in the same line as SEQ ID NO. 7947 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78154, or a homolog or fragment thereof, or a homolog thereof being depicted in Table I, application no. 15, column 8, in the same line as SEQ ID NO. 78154, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 78154 is depicted, plastidic is mentioned, or a mitochondrial transit peptide in case in Table I, application no. 15, in column 6 in the same line as SEQ ID NO. 78154 is depicted, mitochondric is mentioned; which is operable linked, too.

In another embodiment thereof the expression cassette does not comprise an additional transit peptide in case in Table I, application no. 15, in column 6 in the same line as SEQ ID NO. 78154 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115064, or a homolog or fragment thereof, or a homolog thereof being depicted in Table I, application no. 15, column 8, in the same line as SEQ ID NO. 115064, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 115064 is depicted, plastidic is mentioned, or a mitochondrial transit peptide in case in Table I, application no. 15, in column 6 in the same line as SEQ ID NO. 115064 is depicted, mitochondric is mentioned; which is operable linked, too.

In another embodiment thereof the expression cassette does not comprise an additional transit peptide in case in Table I, application no. 15, in column 6 in the same line as SEQ ID NO. 115064 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38345, or a homolog or fragment thereof, or a homolog thereof being depicted in Table I, application no. 15, column 8, in the same line as SEQ ID NO. 38345, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 38345 is depicted, plastidic is mentioned, or a mitochondrial transit peptide in case in Table I, application no. 15, in column 6 in the same line as SEQ ID NO. 38345 is depicted, mitochondric is mentioned; which is operable linked, too.

In another embodiment thereof the expression cassette does not comprise an additional transit peptide in case in Table I, application no. 15, in column 6 in the same line as SEQ ID NO. 38345 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78265, or a homolog or fragment thereof, or a homolog thereof being depicted in Table I, application no. 15, column 8, in the same line as SEQ ID NO. 78265, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 78265 is depicted, plastidic is mentioned, or a mitochondrial transit peptide in case in Table I, application no. 15, in column 6 in the same line as SEQ ID NO. 78265 is depicted, mitochondric is mentioned; which is operable linked, too.

In another embodiment thereof the expression cassette does not comprise an additional transit peptide in case in Table I, application no. 15, in column 6 in the same line as SEQ ID NO. 78265 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78753, or a homolog or fragment thereof, or a homolog thereof being depicted in Table I, application no. 15, column 8, in the same line as SEQ ID NO. 78753, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 78753 is depicted, plastidic is mentioned, or a mitochondrial transit peptide in case in Table I, application no. 15, in column 6 in the same line as SEQ ID NO. 78753 is depicted, mitochondric is mentioned; which is operable linked, too.

In another embodiment thereof the expression cassette does not comprise an additional transit peptide in case in Table I, application no. 15, in column 6 in the same line as SEQ ID NO. 78753 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78771, or a homolog or fragment thereof, or a homolog thereof being depicted in Table I, application no. 15, column 8, in the same line as SEQ ID NO. 78771, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 78771 is depicted, plastidic is mentioned, or a mitochondrial transit peptide in case in Table I, application no. 15, in column 6 in the same line as SEQ ID NO. 78771 is depicted, mitochondric is mentioned; which is operable linked, too.

In another embodiment thereof the expression cassette does not comprise an additional transit peptide in case in Table I, application no. 15, in column 6 in the same line as SEQ ID NO. 78771 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 134762, or a homolog or fragment thereof, or a homolog thereof being depicted in Table I, application no. 15, column 8, in the same line as SEQ ID NO. 134762, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 134762 is depicted, plastidic is mentioned, or a mitochondrial transit peptide in case in Table I, application no. 15, in column 6 in the same line as SEQ ID NO. 134762 is depicted, mitochondric is mentioned; which is operable linked, too.

In another embodiment thereof the expression cassette does not comprise an additional transit peptide in case in Table I, application no. 15, in column 6 in the same line as SEQ ID NO. 134762 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 78779, or a homolog or fragment thereof, or a homolog thereof being depicted in Table I, application no. 15, column 8, in the same line as SEQ ID NO. 78779, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 78779 is depicted, plastidic is mentioned, or a mitochondrial transit peptide in case in Table I, application no. 15, in column 6 in the same line as SEQ ID NO. 78779 is depicted, mitochondric is mentioned; which is operable linked, too.

In another embodiment thereof the expression cassette does not comprise an additional transit peptide in case in Table I, application no. 15, in column 6 in the same line as SEQ ID NO. 78779 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78852, or a homolog or fragment thereof, or a homolog thereof being depicted in Table I, application no. 15, column 8, in the same line as SEQ ID NO. 78852, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 78852 is depicted, plastidic is mentioned, or a mitochondrial transit peptide in case in Table I, application no. 15, in column 6 in the same line as SEQ ID NO. 78852 is depicted, mitochondric is mentioned; which is operable linked, too.

In another embodiment thereof the expression cassette does not comprise an additional transit peptide in case in Table I, application no. 15, in column 6 in the same line as SEQ ID NO. 78852 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38573, or a homolog or fragment thereof, or a homolog thereof being depicted in Table I, application no. 15, column 8, in the same line as SEQ ID NO. 38573, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 38573 is depicted, plastidic is mentioned, or a mitochondrial transit peptide in case in Table I, application no. 15, in column 6 in the same line as SEQ ID NO. 38573 is depicted, mitochondric is mentioned; which is operable linked, too.

In another embodiment thereof the expression cassette does not comprise an additional transit peptide in case in Table I, application no. 15, in column 6 in the same line as SEQ ID NO. 38573 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 134783, or a homolog or fragment thereof, or a homolog thereof being depicted in Table I, application no. 15, column 8, in the same line as SEQ ID NO. 134783, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 134783 is depicted, plastidic is mentioned, or a mitochondrial transit peptide in case in Table I, application no. 15, in column 6 in the same line as SEQ ID NO. 134783 is depicted, mitochondric is mentioned; which is operable linked, too.

In another embodiment thereof the expression cassette does not comprise an additional transit peptide in case in Table I, application no. 15, in column 6 in the same line as SEQ ID NO. 134783 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38767, or a homolog or fragment thereof, or a homolog thereof being depicted in Table I, application no. 15, column 8, in the same line as SEQ ID NO. 38767, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 38767 is depicted, plastidic is mentioned, or a mitochondrial transit peptide in case in Table I, application no. 15, in column 6 in the same line as SEQ ID NO. 38767 is depicted, mitochondric is mentioned; which is operable linked, too.

In another embodiment thereof the expression cassette does not comprise an additional transit peptide in case in Table I, application no. 15, in column 6 in the same line as SEQ ID NO. 38767 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 134864, or a homolog or fragment thereof, or a homolog thereof being depicted in Table I, application no. 15, column 8, in the same line as SEQ ID NO. 134864, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 134864 is depicted, plastidic is mentioned, or a mitochondrial transit peptide in case in Table I, application no. 15, in column 6 in the same line as SEQ ID NO. 134864 is depicted, mitochondric is mentioned; which is operable linked, too.

In another embodiment thereof the expression cassette does not comprise an additional transit peptide in case in Table I, application no. 15, in column 6 in the same line as SEQ ID NO. 134864 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 79217, or a homolog or fragment thereof, or a homolog thereof being depicted in Table I, application no. 15, column 8, in the same line as SEQ ID NO. 79217, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 79217 is depicted, plastidic is mentioned, or a mitochondrial transit peptide in case in Table I, application no. 15, in column 6 in the same line as SEQ ID NO. 79217 is depicted, mitochondric is mentioned; which is operable linked, too.

In another embodiment thereof the expression cassette does not comprise an additional transit peptide in case in Table I, application no. 15, in column 6 in the same line as SEQ ID NO. 79217 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39237, or a homolog or fragment thereof, or a homolog thereof being depicted in Table I, application no. 15, column 8, in the same line as SEQ ID NO. 39237, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 39237 is depicted, plastidic is mentioned, or a mitochondrial transit peptide in case in Table I, application no. 15, in column 6 in the same line as SEQ ID NO. 39237 is depicted, mitochondric is mentioned; which is operable linked, too.

In another embodiment thereof the expression cassette does not comprise an additional transit peptide in case in Table I, application no. 15, in column 6 in the same line as SEQ ID NO. 39237 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39300, or a homolog or fragment thereof, or a homolog thereof being depicted in Table I, application no. 15, column 8, in the same line as SEQ ID NO. 39300, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 39300 is depicted, plastidic is mentioned, or a mitochondrial transit peptide in case in Table I, application no. 15, in column 6 in the same line as SEQ ID NO. 39300 is depicted, mitochondric is mentioned; which is operable linked, too.

In another embodiment thereof the expression cassette does not comprise an additional transit peptide in case in Table I, application no. 15, in column 6 in the same line as SEQ ID NO. 39300 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 134924, or a homolog or fragment thereof, or a homolog thereof being depicted in Table I, application no. 15, column 8, in the same line as SEQ ID NO. 134924, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 134924 is depicted, plastidic is mentioned, or a mitochondrial transit peptide in case in Table I, application no. 15, in column 6 in the same line as SEQ ID NO. 134924 is depicted, mitochondric is mentioned; which is operable linked, too.

In another embodiment thereof the expression cassette does not comprise an additional transit peptide in case in Table I, application no. 15, in column 6 in the same line as SEQ ID NO. 134924 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40726, or a homolog or fragment thereof, or a homolog thereof being depicted in Table I, application no. 15, column 8, in the same line as SEQ ID NO. 40726, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 40726 is depicted, plastidic is mentioned, or a mitochondrial transit peptide in case in Table I, application no. 15, in column 6 in the same line as SEQ ID NO. 40726 is depicted, mitochondric is mentioned; which is operable linked, too.

In another embodiment thereof the expression cassette does not comprise an additional transit peptide in case in Table I, application no. 15, in column 6 in the same line as SEQ ID NO. 40726 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 80756, or a homolog or fragment thereof, or a homolog thereof being depicted in Table I, application no. 15, column 8, in the same line as SEQ ID NO. 80756, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 80756 is depicted, plastidic is mentioned, or a mitochondrial transit peptide in case in Table I, application no. 15, in column 6 in the same line as SEQ ID NO. 80756 is depicted, mitochondric is mentioned; which is operable linked, too.

In another embodiment thereof the expression cassette does not comprise an additional transit peptide in case in Table I, application no. 15, in column 6 in the same line as SEQ ID NO. 80756 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 81075, or a homolog or fragment thereof, or a homolog thereof being depicted in Table I, application no. 15, column 8, in the same line as SEQ ID NO. 81075, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 81075 is depicted, plastidic is mentioned, or a mitochondrial transit peptide in case in Table I, application no. 15, in column 6 in the same line as SEQ ID NO. 81075 is depicted, mitochondric is mentioned; which is operable linked, too.

In another embodiment thereof the expression cassette does not comprise an additional transit peptide in case in Table I, application no. 15, in column 6 in the same line as SEQ ID NO. 81075 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 81576, or a homolog or fragment thereof, or a homolog thereof being depicted in Table I, application no. 15, column 8, in the same line as SEQ ID NO. 81576, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 81576 is depicted, plastidic is mentioned, or a mitochondrial transit peptide in case in Table I, application no. 15, in column 6 in the same line as SEQ ID NO. 81576 is depicted, mitochondric is mentioned; which is operable linked, too.

In another embodiment thereof the expression cassette does not comprise an additional transit peptide in case in Table I, application no. 15, in column 6 in the same line as SEQ ID NO. 81576 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115864, or a homolog or fragment thereof, or a homolog thereof being depicted in Table I, application no. 15, column 8, in the same line as SEQ ID NO. 115864, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 115864 is depicted, plastidic is mentioned, or a mitochondrial transit peptide in case in Table I, application no. 15, in column 6 in the same line as SEQ ID NO. 115864 is depicted, mitochondric is mentioned; which is operable linked, too.

In another embodiment thereof the expression cassette does not comprise an additional transit peptide in case in Table I, application no. 15, in column 6 in the same line as SEQ ID NO. 115864 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 135893, or a homolog or fragment thereof, or a homolog thereof being depicted in Table I, application no. 15, column 8, in the same line as SEQ ID NO. 135893, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 135893 is depicted, plastidic is mentioned, or a mitochondrial transit peptide in case in Table I, application no. 15, in column 6 in the same line as SEQ ID NO. 135893 is depicted, mitochondric is mentioned; which is operable linked, too.

In another embodiment thereof the expression cassette does not comprise an additional transit peptide in case in Table I, application no. 15, in column 6 in the same line as SEQ ID NO. 135893 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 42502, or a homolog or fragment thereof, or a homolog thereof being depicted in Table I, application no. 15, column 8, in the same line as SEQ ID NO. 42502, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 42502 is depicted, plastidic is mentioned, or a mitochondrial transit peptide in case in Table I, application no. 15, in column 6 in the same line as SEQ ID NO. 42502 is depicted, mitochondric is mentioned; which is operable linked, too.

In another embodiment thereof the expression cassette does not comprise an additional transit peptide in case in Table I, application no. 15, in column 6 in the same line as SEQ ID NO. 42502 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 136077, or a homolog or fragment thereof, or a homolog thereof being depicted in Table I, application no. 15, column 8, in the same line as SEQ ID NO. 136077, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 136077 is depicted, plastidic is mentioned, or a mitochondrial transit peptide in case in Table I, application no. 15, in column 6 in the same line as SEQ ID NO. 136077 is depicted, mitochondric is mentioned; which is operable linked, too.

In another embodiment thereof the expression cassette does not comprise an additional transit peptide in case in Table I, application no. 15, in column 6 in the same line as SEQ ID NO. 136077 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 136098, or a homolog or fragment thereof, or a homolog thereof being depicted in Table I, application no. 15, column 8, in the same line as SEQ ID NO. 136098, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 136098 is depicted, plastidic is mentioned, or a mitochondrial transit peptide in case in Table I, application no. 15, in column 6 in the same line as SEQ ID NO. 136098 is depicted, mitochondric is mentioned; which is operable linked, too.

In another embodiment thereof the expression cassette does not comprise an additional transit peptide in case in Table I, application no. 15, in column 6 in the same line as SEQ ID NO. 136098 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 83726, or a homolog or fragment thereof, or a homolog thereof being depicted in Table I, application no. 15, column 8, in the same line as SEQ ID NO. 83726, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 83726 is depicted, plastidic is mentioned, or a mitochondrial transit peptide in case in Table I, application no. 15, in column 6 in the same line as SEQ ID NO. 83726 is depicted, mitochondric is mentioned; which is operable linked, too.

In another embodiment thereof the expression cassette does not comprise an additional transit peptide in case in Table I, application no. 15, in column 6 in the same line as SEQ ID NO. 83726 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 44196, or a homolog or fragment thereof, or a homolog thereof being depicted in Table I, application no. 15, column 8, in the same line as SEQ ID NO. 44196, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 44196 is depicted, plastidic is mentioned, or a mitochondrial transit peptide in case in Table I, application no. 15, in column 6 in the same line as SEQ ID NO. 44196 is depicted, mitochondric is mentioned; which is operable linked, too.

In another embodiment thereof the expression cassette does not comprise an additional transit peptide in case in Table I, application no. 15, in column 6 in the same line as SEQ ID NO. 44196 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 10708, or a homolog or fragment thereof, or a homolog thereof being depicted in Table I, application no. 15, column 8, in the same line as SEQ ID NO. 10708, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 10708 is depicted, plastidic is mentioned, or a mitochondrial transit peptide in case in Table I, application no. 15, in column 6 in the same line as SEQ ID NO. 10708 is depicted, mitochondric is mentioned; which is operable linked, too.

In another embodiment thereof the expression cassette does not comprise an additional transit peptide in case in Table I, application no. 15, in column 6 in the same line as SEQ ID NO. 10708 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 10726, or a homolog or fragment thereof, or a homolog thereof being depicted in Table I, application no. 15, column 8, in the same line as SEQ ID NO. 10726, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 10726 is depicted, plastidic is mentioned, or a mitochondrial transit peptide in case in Table I, application no. 15, in column 6 in the same line as SEQ ID NO. 10726 is depicted, mitochondric is mentioned; which is operable linked, too.

In another embodiment thereof the expression cassette does not comprise an additional transit peptide in case in Table I, application no. 15, in column 6 in the same line as SEQ ID NO. 10726 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 44372, or a homolog or fragment thereof, or a homolog thereof being depicted in Table I, application no. 15, column 8, in the same line as SEQ ID NO. 44372, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 44372 is depicted, plastidic is mentioned, or a mitochondrial transit peptide in case in Table I, application no. 15, in column 6 in the same line as SEQ ID NO. 44372 is depicted, mitochondric is mentioned; which is operable linked, too.

In another embodiment thereof the expression cassette does not comprise an additional transit peptide in case in Table I, application no. 15, in column 6 in the same line as SEQ ID NO. 44372 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 44378, or a homolog or fragment thereof, or a homolog thereof being depicted in Table I, application no. 15, column 8, in the same line as SEQ ID NO. 44378, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 44378 is depicted, plastidic is mentioned, or a mitochondrial transit peptide in case in Table I, application no. 15, in column 6 in the same line as SEQ ID NO. 44378 is depicted, mitochondric is mentioned; which is operable linked, too.

In another embodiment thereof the expression cassette does not comprise an additional transit peptide in case in Table I, application no. 15, in column 6 in the same line as SEQ ID NO. 44378 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 10740, or a homolog or fragment thereof, or a homolog thereof being depicted in Table I, application no. 15, column 8, in the same line as SEQ ID NO. 10740, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 10740 is depicted, plastidic is mentioned, or a mitochondrial transit peptide in case in Table I, application no. 15, in column 6 in the same line as SEQ ID NO. 10740 is depicted, mitochondric is mentioned; which is operable linked, too.

In another embodiment thereof the expression cassette does not comprise an additional transit peptide in case in Table I, application no. 15, in column 6 in the same line as SEQ ID NO. 10740 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 106532, or a homolog or fragment thereof, or a homolog thereof being depicted in Table I, application no. 15, column 8, in the same line as SEQ ID NO. 106532, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 106532 is depicted, plastidic is mentioned, or a mitochondrial transit peptide in case in Table I, application no. 15, in column 6 in the same line as SEQ ID NO. 106532 is depicted, mitochondric is mentioned; which is operable linked, too.

In another embodiment thereof the expression cassette does not comprise an additional transit peptide in case in Table I, application no. 15, in column 6 in the same line as SEQ ID NO. 106532 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 94542, or a homolog or fragment thereof, or a homolog thereof being depicted in Table I, application no. 15, column 8, in the same line as SEQ ID NO. 94542, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 94542 is depicted, plastidic is mentioned, or a mitochondrial transit peptide in case in Table I, application no. 15, in column 6 in the same line as SEQ ID NO. 94542 is depicted, mitochondric is mentioned; which is operable linked, too.

In another embodiment thereof the expression cassette does not comprise an additional transit peptide in case in Table I, application no. 15, in column 6 in the same line as SEQ ID NO. 94542 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 45022, or a homolog or fragment thereof, or a homolog thereof being depicted in Table I, application no. 15, column 8, in the same line as SEQ ID NO. 45022, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 45022 is depicted, plastidic is mentioned, or a mitochondrial transit peptide in case in Table I, application no. 15, in column 6 in the same line as SEQ ID NO. 45022 is depicted, mitochondric is mentioned; which is operable linked, too.

In another embodiment thereof the expression cassette does not comprise an additional transit peptide in case in Table I, application no. 15, in column 6 in the same line as SEQ ID NO. 45022 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 45321, or a homolog or fragment thereof, or a homolog thereof being depicted in Table I, application no. 15, column 8, in the same line as SEQ ID NO. 45321, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 45321 is depicted, plastidic is mentioned, or a mitochondrial transit peptide in case in Table I, application no. 15, in column 6 in the same line as SEQ ID NO. 45321 is depicted, mitochondric is mentioned; which is operable linked, too.

In another embodiment thereof the expression cassette does not comprise an additional transit peptide in case in Table I, application no. 15, in column 6 in the same line as SEQ ID NO. 45321 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 45556, or a homolog or fragment thereof, or a homolog thereof being depicted in Table I, application no. 15, column 8, in the same line as SEQ ID NO. 45556, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 45556 is depicted, plastidic is mentioned, or a mitochondrial transit peptide in case in Table I, application no. 15, in column 6 in the same line as SEQ ID NO. 45556 is depicted, mitochondric is mentioned; which is operable linked, too.

In another embodiment thereof the expression cassette does not comprise an additional transit peptide in case in Table I, application no. 15, in column 6 in the same line as SEQ ID NO. 45556 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 84079, or a homolog or fragment thereof, or a homolog thereof being depicted in Table I, application no. 15, column 8, in the same line as SEQ ID NO. 84079, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 84079 is depicted, plastidic is mentioned, or a mitochondrial transit peptide in case in Table I, application no. 15, in column 6 in the same line as SEQ ID NO. 84079 is depicted, mitochondric is mentioned; which is operable linked, too.

In another embodiment thereof the expression cassette does not comprise an additional transit peptide in case in Table I, application no. 15, in column 6 in the same line as SEQ ID NO. 84079 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 10811, or a homolog or fragment thereof, or a homolog thereof being depicted in Table I, application no. 15, column 8, in the same line as SEQ ID NO. 10811, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 10811 is depicted, plastidic is mentioned, or a mitochondrial transit peptide in case in Table I, application no. 15, in column 6 in the same line as SEQ ID NO. 10811 is depicted, mitochondric is mentioned; which is operable linked, too.

In another embodiment thereof the expression cassette does not comprise an additional transit peptide in case in Table I, application no. 15, in column 6 in the same line as SEQ ID NO. 10811 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 46405, or a homolog or fragment thereof, or a homolog thereof being depicted in Table I, application no. 15, column 8, in the same line as SEQ ID NO. 46405, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 46405 is depicted, plastidic is mentioned, or a mitochondrial transit peptide in case in Table I, application no. 15, in column 6 in the same line as SEQ ID NO. 46405 is depicted, mitochondric is mentioned; which is operable linked, too.

In another embodiment thereof the expression cassette does not comprise an additional transit peptide in case in Table I, application no. 15, in column 6 in the same line as SEQ ID NO. 46405 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 84198, or a homolog or fragment thereof, or a homolog thereof being depicted in Table I, application no. 15, column 8, in the same line as SEQ ID NO. 84198, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 84198 is depicted, plastidic is mentioned, or a mitochondrial transit peptide in case in Table I, application no. 15, in column 6 in the same line as SEQ ID NO. 84198 is depicted, mitochondric is mentioned; which is operable linked, too.

In another embodiment thereof the expression cassette does not comprise an additional transit peptide in case in Table I, application no. 15, in column 6 in the same line as SEQ ID NO. 84198 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 47076, or a homolog or fragment thereof, or a homolog thereof being depicted in Table I, application no. 15, column 8, in the same line as SEQ ID NO. 47076, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 47076 is depicted, plastidic is mentioned, or a mitochondrial transit peptide in case in Table I, application no. 15, in column 6 in the same line as SEQ ID NO. 47076 is depicted, mitochondric is mentioned; which is operable linked, too.

In another embodiment thereof the expression cassette does not comprise an additional transit peptide in case in Table I, application no. 15, in column 6 in the same line as SEQ ID NO. 47076 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 48138, or a homolog or fragment thereof, or a homolog thereof being depicted in Table I, application no. 15, column 8, in the same line as SEQ ID NO. 48138, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 48138 is depicted, plastidic is mentioned, or a mitochondrial transit peptide in case in Table I, application no. 15, in column 6 in the same line as SEQ ID NO. 48138 is depicted, mitochondric is mentioned; which is operable linked, too.

In another embodiment thereof the expression cassette does not comprise an additional transit peptide in case in Table I, application no. 15, in column 6 in the same line as SEQ ID NO. 48138 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 84265, or a homolog or fragment thereof, or a homolog thereof being depicted in Table I, application no. 15, column 8, in the same line as SEQ ID NO. 84265, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 84265 is depicted, plastidic is mentioned, or a mitochondrial transit peptide in case in Table I, application no. 15, in column 6 in the same line as SEQ ID NO. 84265 is depicted, mitochondric is mentioned; which is operable linked, too.

In another embodiment thereof the expression cassette does not comprise an additional transit peptide in case in Table I, application no. 15, in column 6 in the same line as SEQ ID NO. 84265 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 84441, or a homolog or fragment thereof, or a homolog thereof being depicted in Table I, application no. 15, column 8, in the same line as SEQ ID NO. 84441, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 84441 is depicted, plastidic is mentioned, or a mitochondrial transit peptide in case in Table I, application no. 15, in column 6 in the same line as SEQ ID NO. 84441 is depicted, mitochondric is mentioned; which is operable linked, too.

In another embodiment thereof the expression cassette does not comprise an additional transit peptide in case in Table I, application no. 15, in column 6 in the same line as SEQ ID NO. 84441 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 51198, or a homolog or fragment thereof, or a homolog thereof being depicted in Table I, application no. 15, column 8, in the same line as SEQ ID NO. 51198, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 51198 is depicted, plastidic is mentioned, or a mitochondrial transit peptide in case in Table I, application no. 15, in column 6 in the same line as SEQ ID NO. 51198 is depicted, mitochondric is mentioned; which is operable linked, too.

In another embodiment thereof the expression cassette does not comprise an additional transit peptide in case in Table I, application no. 15, in column 6 in the same line as SEQ ID NO. 51198 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 127109, or a homolog or fragment thereof, or a homolog thereof being depicted in Table I, application no. 15, column 8, in the same line as SEQ ID NO. 127109, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 127109 is depicted, plastidic is mentioned, or a mitochondrial transit peptide in case in Table I, application no. 15, in column 6 in the same line as SEQ ID NO. 127109 is depicted, mitochondric is mentioned; which is operable linked, too.

In another embodiment thereof the expression cassette does not comprise an additional transit peptide in case in Table I, application no. 15, in column 6 in the same line as SEQ ID NO. 127109 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 54452, or a homolog or fragment thereof, or a homolog thereof being depicted in Table I, application no. 15, column 8, in the same line as SEQ ID NO. 54452, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 54452 is depicted, plastidic is mentioned, or a mitochondrial transit peptide in case in Table I, application no. 15, in column 6 in the same line as SEQ ID NO. 54452 is depicted, mitochondric is mentioned; which is operable linked, too.

In another embodiment thereof the expression cassette does not comprise an additional transit peptide in case in Table I, application no. 15, in column 6 in the same line as SEQ ID NO. 54452 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 85808, or a homolog or fragment thereof, or a homolog thereof being depicted in Table I, application no. 15, column 8, in the same line as SEQ ID NO. 85808, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 85808 is depicted, plastidic is mentioned, or a mitochondrial transit peptide in case in Table I, application no. 15, in column 6 in the same line as SEQ ID NO. 85808 is depicted, mitochondric is mentioned; which is operable linked, too.

In another embodiment thereof the expression cassette does not comprise an additional transit peptide in case in Table I, application no. 15, in column 6 in the same line as SEQ ID NO. 85808 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 55385, or a homolog or fragment thereof, or a homolog thereof being depicted in Table I, application no. 15, column 8, in the same line as SEQ ID NO. 55385, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 55385 is depicted, plastidic is mentioned, or a mitochondrial transit peptide in case in Table I, application no. 15, in column 6 in the same line as SEQ ID NO. 55385 is depicted, mitochondric is mentioned; which is operable linked, too.

In another embodiment thereof the expression cassette does not comprise an additional transit peptide in case in Table I, application no. 15, in column 6 in the same line as SEQ ID NO. 55385 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 136533, or a homolog or fragment thereof, or a homolog thereof being depicted in Table I, application no. 15, column 8, in the same line as SEQ ID NO. 136533, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 136533 is depicted, plastidic is mentioned, or a mitochondrial transit peptide in case in Table I, application no. 15, in column 6 in the same line as SEQ ID NO. 136533 is depicted, mitochondric is mentioned; which is operable linked, too.

In another embodiment thereof the expression cassette does not comprise an additional transit peptide in case in Table I, application no. 15, in column 6 in the same line as SEQ ID NO. 136533 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 11990, or a homolog or fragment thereof, or a homolog thereof being depicted in Table I, application no. 15, column 8, in the same line as SEQ ID NO. 11990, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 11990 is depicted, plastidic is mentioned, or a mitochondrial transit peptide in case in Table I, application no. 15, in column 6 in the same line as SEQ ID NO. 11990 is depicted, mitochondric is mentioned; which is operable linked, too.

In another embodiment thereof the expression cassette does not comprise an additional transit peptide in case in Table I, application no. 15, in column 6 in the same line as SEQ ID NO. 11990 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 56153, or a homolog or fragment thereof, or a homolog thereof being depicted in Table I, application no. 15, column 8, in the same line as SEQ ID NO. 56153, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 56153 is depicted, plastidic is mentioned, or a mitochondrial transit peptide in case in Table I, application no. 15, in column 6 in the same line as SEQ ID NO. 56153 is depicted, mitochondric is mentioned; which is operable linked, too.

In another embodiment thereof the expression cassette does not comprise an additional transit peptide in case in Table I, application no. 15, in column 6 in the same line as SEQ ID NO. 56153 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 87397, or a homolog or fragment thereof, or a homolog thereof being depicted in Table I, application no. 15, column 8, in the same line as SEQ ID NO. 87397, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 87397 is depicted, plastidic is mentioned, or a mitochondrial transit peptide in case in Table I, application no. 15, in column 6 in the same line as SEQ ID NO. 87397 is depicted, mitochondric is mentioned; which is operable linked, too.

In another embodiment thereof the expression cassette does not comprise an additional transit peptide in case in Table I, application no. 15, in column 6 in the same line as SEQ ID NO. 87397 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 57734, or a homolog or fragment thereof, or a homolog thereof being depicted in Table I, application no. 15, column 8, in the same line as SEQ ID NO. 57734, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 57734 is depicted, plastidic is mentioned, or a mitochondrial transit peptide in case in Table I, application no. 15, in column 6 in the same line as SEQ ID NO. 57734 is depicted, mitochondric is mentioned; which is operable linked, too.

In another embodiment thereof the expression cassette does not comprise an additional transit peptide in case in Table I, application no. 15, in column 6 in the same line as SEQ ID NO. 57734 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 136869, or a homolog or fragment thereof, or a homolog thereof being depicted in Table I, application no. 15, column 8, in the same line as SEQ ID NO. 136869, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 136869 is depicted, plastidic is mentioned, or a mitochondrial transit peptide in case in Table I, application no. 15, in column 6 in the same line as SEQ ID NO. 136869 is depicted, mitochondric is mentioned; which is operable linked, too.

In another embodiment thereof the expression cassette does not comprise an additional transit peptide in case in Table I, application no. 15, in column 6 in the same line as SEQ ID NO. 136869 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 116460, or a homolog or fragment thereof, or a homolog thereof being depicted in Table I, application no. 15, column 8, in the same line as SEQ ID NO. 116460, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 116460 is depicted, plastidic is mentioned, or a mitochondrial transit peptide in case in Table I, application no. 15, in column 6 in the same line as SEQ ID NO. 116460 is depicted, mitochondric is mentioned; which is operable linked, too.

In another embodiment thereof the expression cassette does not comprise an additional transit peptide in case in Table I, application no. 15, in column 6 in the same line as SEQ ID NO. 116460 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 58058, or a homolog or fragment thereof, or a homolog thereof being depicted in Table I, application no. 15, column 8, in the same line as SEQ ID NO. 58058, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 58058 is depicted, plastidic is mentioned, or a mitochondrial transit peptide in case in Table I, application no. 15, in column 6 in the same line as SEQ ID NO. 58058 is depicted, mitochondric is mentioned; which is operable linked, too.

In another embodiment thereof the expression cassette does not comprise an additional transit peptide in case in Table I, application no. 15, in column 6 in the same line as SEQ ID NO. 58058 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 58731, or a homolog or fragment thereof, or a homolog thereof being depicted in Table I, application no. 15, column 8, in the same line as SEQ ID NO. 58731, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 58731 is depicted, plastidic is mentioned, or a mitochondrial transit peptide in case in Table I, application no. 15, in column 6 in the same line as SEQ ID NO. 58731 is depicted, mitochondric is mentioned; which is operable linked, too.

In another embodiment thereof the expression cassette does not comprise an additional transit peptide in case in Table I, application no. 15, in column 6 in the same line as SEQ ID NO. 58731 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 59165, or a homolog or fragment thereof, or a homolog thereof being depicted in Table I, application no. 15, column 8, in the same line as SEQ ID NO. 59165, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 59165 is depicted, plastidic is mentioned, or a mitochondrial transit peptide in case in Table I, application no. 15, in column 6 in the same line as SEQ ID NO. 59165 is depicted, mitochondric is mentioned; which is operable linked, too.

In another embodiment thereof the expression cassette does not comprise an additional transit peptide in case in Table I, application no. 15, in column 6 in the same line as SEQ ID NO. 59165 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 136907, or a homolog or fragment thereof, or a homolog thereof being depicted in Table I, application no. 15, column 8, in the same line as SEQ ID NO. 136907, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 136907 is depicted, plastidic is mentioned, or a mitochondrial transit peptide in case in Table I, application no. 15, in column 6 in the same line as SEQ ID NO. 136907 is depicted, mitochondric is mentioned; which is operable linked, too.

In another embodiment thereof the expression cassette does not comprise an additional transit peptide in case in Table I, application no. 15, in column 6 in the same line as SEQ ID NO. 136907 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12698, or a homolog or fragment thereof, or a homolog thereof being depicted in Table I, application no. 15, column 8, in the same line as SEQ ID NO. 12698, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 12698 is depicted, plastidic is mentioned, or a mitochondrial transit peptide in case in Table I, application no. 15, in column 6 in the same line as SEQ ID NO. 12698 is depicted, mitochondric is mentioned; which is operable linked, too.

In another embodiment thereof the expression cassette does not comprise an additional transit peptide in case in Table I, application no. 15, in column 6 in the same line as SEQ ID NO. 12698 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 107556, or a homolog or fragment thereof, or a homolog thereof being depicted in Table I, application no. 15, column 8, in the same line as SEQ ID NO. 107556, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 107556 is depicted, plastidic is mentioned, or a mitochondrial transit peptide in case in Table I, application no. 15, in column 6 in the same line as SEQ ID NO. 107556 is depicted, mitochondric is mentioned; which is operable linked, too.

In another embodiment thereof the expression cassette does not comprise an additional transit peptide in case in Table I, application no. 15, in column 6 in the same line as SEQ ID NO. 107556 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 61723, or a homolog or fragment thereof, or a homolog thereof being depicted in Table I, application no. 15, column 8, in the same line as SEQ ID NO. 61723, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 61723 is depicted, plastidic is mentioned, or a mitochondrial transit peptide in case in Table I, application no. 15, in column 6 in the same line as SEQ ID NO. 61723 is depicted, mitochondric is mentioned; which is operable linked, too.

In another embodiment thereof the expression cassette does not comprise an additional transit peptide in case in Table I, application no. 15, in column 6 in the same line as SEQ ID NO. 61723 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 62079, or a homolog or fragment thereof, or a homolog thereof being depicted in Table I, application no. 15, column 8, in the same line as SEQ ID NO. 62079, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 62079 is depicted, plastidic is mentioned, or a mitochondrial transit peptide in case in Table I, application no. 15, in column 6 in the same line as SEQ ID NO. 62079 is depicted, mitochondric is mentioned; which is operable linked, too.

In another embodiment thereof the expression cassette does not comprise an additional transit peptide in case in Table I, application no. 15, in column 6 in the same line as SEQ ID NO. 62079 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 62160, or a homolog or fragment thereof, or a homolog thereof being depicted in Table I, application no. 15, column 8, in the same line as SEQ ID NO. 62160, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 62160 is depicted, plastidic is mentioned, or a mitochondrial transit peptide in case in Table I, application no. 15, in column 6 in the same line as SEQ ID NO. 62160 is depicted, mitochondric is mentioned; which is operable linked, too.

In another embodiment thereof the expression cassette does not comprise an additional transit peptide in case in Table I, application no. 15, in column 6 in the same line as SEQ ID NO. 62160 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 62244, or a homolog or fragment thereof, or a homolog thereof being depicted in Table I, application no. 15, column 8, in the same line as SEQ ID NO. 62244, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 62244 is depicted, plastidic is mentioned, or a mitochondrial transit peptide in case in Table I, application no. 15, in column 6 in the same line as SEQ ID NO. 62244 is depicted, mitochondric is mentioned; which is operable linked, too.

In another embodiment thereof the expression cassette does not comprise an additional transit peptide in case in Table I, application no. 15, in column 6 in the same line as SEQ ID NO. 62244 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 136943, or a homolog or fragment thereof, or a homolog thereof being depicted in Table I, application no. 15, column 8, in the same line as SEQ ID NO. 136943, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 136943 is depicted, plastidic is mentioned, or a mitochondrial transit peptide in case in Table I, application no. 15, in column 6 in the same line as SEQ ID NO. 136943 is depicted, mitochondric is mentioned; which is operable linked, too.

In another embodiment thereof the expression cassette does not comprise an additional transit peptide in case in Table I, application no. 15, in column 6 in the same line as SEQ ID NO. 136943 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 137387, or a homolog or fragment thereof, or a homolog thereof being depicted in Table I, application no. 15, column 8, in the same line as SEQ ID NO. 137387, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 137387 is depicted, plastidic is mentioned, or a mitochondrial transit peptide in case in Table I, application no. 15, in column 6 in the same line as SEQ ID NO. 137387 is depicted, mitochondric is mentioned; which is operable linked, too.

In another embodiment thereof the expression cassette does not comprise an additional transit peptide in case in Table I, application no. 15, in column 6 in the same line as SEQ ID NO. 137387 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 96779, or a homolog or fragment thereof, or a homolog thereof being depicted in Table I, application no. 15, column 8, in the same line as SEQ ID NO. 96779, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 96779 is depicted, plastidic is mentioned, or a mitochondrial transit peptide in case in Table I, application no. 15, in column 6 in the same line as SEQ ID NO. 96779 is depicted, mitochondric is mentioned; which is operable linked, too.

In another embodiment thereof the expression cassette does not comprise an additional transit peptide in case in Table I, application no. 15, in column 6 in the same line as SEQ ID NO. 96779 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 63264, or a homolog or fragment thereof, or a homolog thereof being depicted in Table I, application no. 15, column 8, in the same line as SEQ ID NO. 63264, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 63264 is depicted, plastidic is mentioned, or a mitochondrial transit peptide in case in Table I, application no. 15, in column 6 in the same line as SEQ ID NO. 63264 is depicted, mitochondric is mentioned; which is operable linked, too.

In another embodiment thereof the expression cassette does not comprise an additional transit peptide in case in Table I, application no. 15, in column 6 in the same line as SEQ ID NO. 63264 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 137498, or a homolog or fragment thereof, or a homolog thereof being depicted in Table I, application no. 15, column 8, in the same line as SEQ ID NO. 137498, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 137498 is depicted, plastidic is mentioned, or a mitochondrial transit peptide in case in Table I, application no. 15, in column 6 in the same line as SEQ ID NO. 137498 is depicted, mitochondric is mentioned; which is operable linked, too.

In another embodiment thereof the expression cassette does not comprise an additional transit peptide in case in Table I, application no. 15, in column 6 in the same line as SEQ ID NO. 137498 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 63334, or a homolog or fragment thereof, or a homolog thereof being depicted in Table I, application no. 15, column 8, in the same line as SEQ ID NO. 63334, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 63334 is depicted, plastidic is mentioned, or a mitochondrial transit peptide in case in Table I, application no. 15, in column 6 in the same line as SEQ ID NO. 63334 is depicted, mitochondric is mentioned; which is operable linked, too.

In another embodiment thereof the expression cassette does not comprise an additional transit peptide in case in Table I, application no. 15, in column 6 in the same line as SEQ ID NO. 63334 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 64177, or a homolog or fragment thereof, or a homolog thereof being depicted in Table I, application no. 15, column 8, in the same line as SEQ ID NO. 64177, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 64177 is depicted, plastidic is mentioned, or a mitochondrial transit peptide in case in Table I, application no. 15, in column 6 in the same line as SEQ ID NO. 64177 is depicted, mitochondric is mentioned; which is operable linked, too.

In another embodiment thereof the expression cassette does not comprise an additional transit peptide in case in Table I, application no. 15, in column 6 in the same line as SEQ ID NO. 64177 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 108487, or a homolog or fragment thereof, or a homolog thereof being depicted in Table I, application no. 15, column 8, in the same line as SEQ ID NO. 108487, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 108487 is depicted, plastidic is mentioned, or a mitochondrial transit peptide in case in Table I, application no. 15, in column 6 in the same line as SEQ ID NO. 108487 is depicted, mitochondric is mentioned; which is operable linked, too.

In another embodiment thereof the expression cassette does not comprise an additional transit peptide in case in Table I, application no. 15, in column 6 in the same line as SEQ ID NO. 108487 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 64315, or a homolog or fragment thereof, or a homolog thereof being depicted in Table I, application no. 15, column 8, in the same line as SEQ ID NO. 64315, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 64315 is depicted, plastidic is mentioned, or a mitochondrial transit peptide in case in Table I, application no. 15, in column 6 in the same line as SEQ ID NO. 64315 is depicted, mitochondric is mentioned; which is operable linked, too.

In another embodiment thereof the expression cassette does not comprise an additional transit peptide in case in Table I, application no. 15, in column 6 in the same line as SEQ ID NO. 64315 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 101855, or a homolog or fragment thereof, or a homolog thereof being depicted in Table I, application no. 15, column 8, in the same line as SEQ ID NO. 101855, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 101855 is depicted, plastidic is mentioned, or a mitochondrial transit peptide in case in Table I, application no. 15, in column 6 in the same line as SEQ ID NO. 101855 is depicted, mitochondric is mentioned; which is operable linked, too.

In another embodiment thereof the expression cassette does not comprise an additional transit peptide in case in Table I, application no. 15, in column 6 in the same line as SEQ ID NO. 101855 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 97620, or a homolog or fragment thereof, or a homolog thereof being depicted in Table I, application no. 15, column 8, in the same line as SEQ ID NO. 97620, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 97620 is depicted, plastidic is mentioned, or a mitochondrial transit peptide in case in Table I, application no. 15, in column 6 in the same line as SEQ ID NO. 97620 is depicted, mitochondric is mentioned; which is operable linked, too.

In another embodiment thereof the expression cassette does not comprise an additional transit peptide in case in Table I, application no. 15, in column 6 in the same line as SEQ ID NO. 97620 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 64546, or a homolog or fragment thereof, or a homolog thereof being depicted in Table I, application no. 15, column 8, in the same line as SEQ ID NO. 64546, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 64546 is depicted, plastidic is mentioned, or a mitochondrial transit peptide in case in Table I, application no. 15, in column 6 in the same line as SEQ ID NO. 64546 is depicted, mitochondric is mentioned; which is operable linked, too.

In another embodiment thereof the expression cassette does not comprise an additional transit peptide in case in Table I, application no. 15, in column 6 in the same line as SEQ ID NO. 64546 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 64964, or a homolog or fragment thereof, or a homolog thereof being depicted in Table I, application no. 15, column 8, in the same line as SEQ ID NO. 64964, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 64964 is depicted, plastidic is mentioned, or a mitochondrial transit peptide in case in Table I, application no. 15, in column 6 in the same line as SEQ ID NO. 64964 is depicted, mitochondric is mentioned; which is operable linked, too.

In another embodiment thereof the expression cassette does not comprise an additional transit peptide in case in Table I, application no. 15, in column 6 in the same line as SEQ ID NO. 64964 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 97635, or a homolog or fragment thereof, or a homolog thereof being depicted in Table I, application no. 15, column 8, in the same line as SEQ ID NO. 97635, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 97635 is depicted, plastidic is mentioned, or a mitochondrial transit peptide in case in Table I, application no. 15, in column 6 in the same line as SEQ ID NO. 97635 is depicted, mitochondric is mentioned; which is operable linked, too.

In another embodiment thereof the expression cassette does not comprise an additional transit peptide in case in Table I, application no. 15, in column 6 in the same line as SEQ ID NO. 97635 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 90409, or a homolog or fragment thereof, or a homolog thereof being depicted in Table I, application no. 15, column 8, in the same line as SEQ ID NO. 90409, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 90409 is depicted, plastidic is mentioned, or a mitochondrial transit peptide in case in Table I, application no. 15, in column 6 in the same line as SEQ ID NO. 90409 is depicted, mitochondric is mentioned; which is operable linked, too.

In another embodiment thereof the expression cassette does not comprise an additional transit peptide in case in Table I, application no. 15, in column 6 in the same line as SEQ ID NO. 90409 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 66274, or a homolog or fragment thereof, or a homolog thereof being depicted in Table I, application no. 15, column 8, in the same line as SEQ ID NO. 66274, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 66274 is depicted, plastidic is mentioned, or a mitochondrial transit peptide in case in Table I, application no. 15, in column 6 in the same line as SEQ ID NO. 66274 is depicted, mitochondric is mentioned; which is operable linked, too.

In another embodiment thereof the expression cassette does not comprise an additional transit peptide in case in Table I, application no. 15, in column 6 in the same line as SEQ ID NO. 66274 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 90602, or a homolog or fragment thereof, or a homolog thereof being depicted in Table I, application no. 15, column 8, in the same line as SEQ ID NO. 90602, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 90602 is depicted, plastidic is mentioned, or a mitochondrial transit peptide in case in Table I, application no. 15, in column 6 in the same line as SEQ ID NO. 90602 is depicted, mitochondric is mentioned; which is operable linked, too.

In another embodiment thereof the expression cassette does not comprise an additional transit peptide in case in Table I, application no. 15, in column 6 in the same line as SEQ ID NO. 90602 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 137502, or a homolog or fragment thereof, or a homolog thereof being depicted in Table I, application no. 15, column 8, in the same line as SEQ ID NO. 137502, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 137502 is depicted, plastidic is mentioned, or a mitochondrial transit peptide in case in Table I, application no. 15, in column 6 in the same line as SEQ ID NO. 137502 is depicted, mitochondric is mentioned; which is operable linked, too.

In another embodiment thereof the expression cassette does not comprise an additional transit peptide in case in Table I, application no. 15, in column 6 in the same line as SEQ ID NO. 137502 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 66695, or a homolog or fragment thereof, or a homolog thereof being depicted in Table I, application no. 15, column 8, in the same line as SEQ ID NO. 66695, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 66695 is depicted, plastidic is mentioned, or a mitochondrial transit peptide in case in Table I, application no. 15, in column 6 in the same line as SEQ ID NO. 66695 is depicted, mitochondric is mentioned; which is operable linked, too.

In another embodiment thereof the expression cassette does not comprise an additional transit peptide in case in Table I, application no. 15, in column 6 in the same line as SEQ ID NO. 66695 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 137547, or a homolog or fragment thereof, or a homolog thereof being depicted in Table I, application no. 15, column 8, in the same line as SEQ ID NO. 137547, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 137547 is depicted, plastidic is mentioned, or a mitochondrial transit peptide in case in Table I, application no. 15, in column 6 in the same line as SEQ ID NO. 137547 is depicted, mitochondric is mentioned; which is operable linked, too.

In another embodiment thereof the expression cassette does not comprise an additional transit peptide in case in Table I, application no. 15, in column 6 in the same line as SEQ ID NO. 137547 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 129123, or a homolog or fragment thereof, or a homolog thereof being depicted in Table I, application no. 15, column 8, in the same line as SEQ ID NO. 129123, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 129123 is depicted, plastidic is mentioned, or a mitochondrial transit peptide in case in Table I, application no. 15, in column 6 in the same line as SEQ ID NO. 129123 is depicted, mitochondric is mentioned; which is operable linked, too.

In another embodiment thereof the expression cassette does not comprise an additional transit peptide in case in Table I, application no. 15, in column 6 in the same line as SEQ ID NO. 129123 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 15175, or a homolog or fragment thereof, or a homolog thereof being depicted in Table I, application no. 15, column 8, in the same line as SEQ ID NO. 15175, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 15175 is depicted, plastidic is mentioned, or a mitochondrial transit peptide in case in Table I, application no. 15, in column 6 in the same line as SEQ ID NO. 15175 is depicted, mitochondric is mentioned; which is operable linked, too.

In another embodiment thereof the expression cassette does not comprise an additional transit peptide in case in Table I, application no. 15, in column 6 in the same line as SEQ ID NO. 15175 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 137818, or a homolog or fragment thereof, or a homolog thereof being depicted in Table I, application no. 15, column 8, in the same line as SEQ ID NO. 137818, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 137818 is depicted, plastidic is mentioned, or a mitochondrial transit peptide in case in Table I, application no. 15, in column 6 in the same line as SEQ ID NO. 137818 is depicted, mitochondric is mentioned; which is operable linked, too.

In another embodiment thereof the expression cassette does not comprise an additional transit peptide in case in Table I, application no. 15, in column 6 in the same line as SEQ ID NO. 137818 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 15179, or a homolog or fragment thereof, or a homolog thereof being depicted in Table I, application no. 15, column 8, in the same line as SEQ ID NO. 15179, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 15179 is depicted, plastidic is mentioned, or a mitochondrial transit peptide in case in Table I, application no. 15, in column 6 in the same line as SEQ ID NO. 15179 is depicted, mitochondric is mentioned; which is operable linked, too.

In another embodiment thereof the expression cassette does not comprise an additional transit peptide in case in Table I, application no. 15, in column 6 in the same line as SEQ ID NO. 15179 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 92668, or a homolog or fragment thereof, or a homolog thereof being depicted in Table I, application no. 15, column 8, in the same line as SEQ ID NO. 92668, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 92668 is depicted, plastidic is mentioned, or a mitochondrial transit peptide in case in Table I, application no. 15, in column 6 in the same line as SEQ ID NO. 92668 is depicted, mitochondric is mentioned; which is operable linked, too.

In another embodiment thereof the expression cassette does not comprise an additional transit peptide in case in Table I, application no. 15, in column 6 in the same line as SEQ ID NO. 92668 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 93218, or a homolog or fragment thereof, or a homolog thereof being depicted in Table I, application no. 15, column 8, in the same line as SEQ ID NO. 93218, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 93218 is depicted, plastidic is mentioned, or a mitochondrial transit peptide in case in Table I, application no. 15, in column 6 in the same line as SEQ ID NO. 93218 is depicted, mitochondric is mentioned; which is operable linked, too.

In another embodiment thereof the expression cassette does not comprise an additional transit peptide in case in Table I, application no. 15, in column 6 in the same line as SEQ ID NO. 93218 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 15187, or a homolog or fragment thereof, or a homolog thereof being depicted in Table I, application no. 15, column 8, in the same line as SEQ ID NO. 15187, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 15187 is depicted, plastidic is mentioned, or a mitochondrial transit peptide in case in Table I, application no. 15, in column 6 in the same line as SEQ ID NO. 15187 is depicted, mitochondric is mentioned; which is operable linked, too.

In another embodiment thereof the expression cassette does not comprise an additional transit peptide in case in Table I, application no. 15, in column 6 in the same line as SEQ ID NO. 15187 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 69, or a homolog or fragment thereof, or a homolog thereof being depicted in Table I, application no. 15, column 8, in the same line as SEQ ID NO. 69, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 69 is depicted, plastidic is mentioned, or a mitochondrial transit peptide in case in Table I, application no. 15, in column 6 in the same line as SEQ ID NO. 69 is depicted, mitochondric is mentioned; which is operable limiant,them.embodiment thereof the expression cassette does not comprise an additional transit peptide in case in Table I, application no. 15, in column 6 in the same line as SEQ ID NO. 69 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 15532, or a homolog or fragment thereof, or a homolog thereof being depicted in Table I, application no. 15, column 8, in the same line as SEQ ID NO. 15532, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 15532 is depicted, plastidic is mentioned, or a mitochondrial transit peptide in case in Table I, application no. 15, in column 6 in the same line as SEQ ID NO. 15532 is depicted, mitochondric is mentioned; which is operable linked, too.

In another embodiment thereof the expression cassette does not comprise an additional transit peptide in case in Table I, application no. 15, in column 6 in the same line as SEQ ID NO. 15532 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 16883, or a homolog or fragment thereof, or a homolog thereof being depicted in Table I, application no. 15, column 8, in the same line as SEQ ID NO. 16883, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 15, in column 6 in the same line as SEQ ID NO. 16883 is depicted, plastidic is mentioned, or a mitochondrial transit peptide in case in Table I, application no. 15, in column 6 in the same line as SEQ ID NO. 16883 is depicted, mitochondric is mentioned; which is operable linked, too.

In another embodiment thereof the expression cassette does not comprise an additional transit peptide in case in Table I, application no. 15, in column 6 in the same line as SEQ ID NO. 16883 is depicted, non-targeted is mentioned.

[0165.1.7.15] to [0170.1.7.15] for the disclosure of these paragraphs see [0165.1.7.7] to [0170.1.7.7] above

The invention further provides a vector, comprising a nucleic acid molecule comprising 1) a nucleic acid molecule selected from the group consisting of:
(a) a nucleic acid molecule encoding a polypeptide shown in Table II, column 5 or 8, application no. 15, preferably shown in Table II A, application no. 15, in column 5, or in Table II A, application no. 15, column 8, or in Table II B, application no. 15, column 8, or a homolog or a fragment thereof;
(b) a nucleic acid molecule shown in Table I, application no. 15, column 5 or 8, preferably shown in Table I A, application no. 15, in column 5, or in Table I A, application no. 15, column 8, or in Table I B, application no. 15, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 15, preferably shown in Table II A, application no. 15, in column 5, or in Table II A, application no. 15, column 8, or in Table II B, application no. 15, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 15, preferably shown in Table I A, application no. 15, in column 5, or in Table I A, application no. 15, column 8, or in Table I B, application no. 15, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 15;
(i) a nucleic acid molecule encoding a polypeptide having the activity represented by a protein as depicted in column 5 of Table II, application no. 15;
(j) a nucleic acid molecule comprising a polynucleotide which is obtained by amplifying a cDNA library or a genomic library using the primers in Table III, application no. 15, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 15.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wildtype non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 15, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 15.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 15, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 15, in the same line as of the respective nucleic acid molecule according to 1) “plastidic” is depicted, or encoding a mitochondrial transit peptide, especially in case in column 6 of Table I, application no. 15, in the same line as of the respective nucleic acid molecule according to 1) “mitochondric” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 15, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 15, in the same line as of the respective nucleic acid molecule according to 1) “plastidic” is depicted, or encoding a mitochondrial transit peptide, especially in case in column 6 of Table I, application no. 15, in the same line as of the respective nucleic acid molecule according to 1) “mitochondric” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operable linked; a nucleic acid molecule encoding a transit peptide, especially in case in column 6 of Table I, application no. 15, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 15, in the same line as of the respective nucleic acid molecule according to 1) “plastidic” is depicted, or encoding a mitochondrial transit peptide, especially in case in column 6 of Table I, application no. 15, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 15 (in the same line).

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 15, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 15.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 15, in the same line as of the respective nucleic acid molecule according to 1) “plastidic” is depicted, or encoding a mitochondrial transit peptide, especially in case in column 6 of Table I, application no. 15, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 15, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 15, in the same line as of the respective nucleic acid molecule according to 1) “plastidic” is depicted, or encoding a mitochondrial transit peptide, especially in case in column 6 of Table I, application no. 15, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 15 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 15, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 15 (in the same line) and confers a generation or an increase of the respective fine chemical in a non-human organism or a part thereof as compared to a corresponding wild-type non-human organism or part thereof.

In a preferred embodiment of said vector said nucleic acid molecule according to 1 (a), (b), (c), (d), (e), (f), (g), (h), (i), (j) or (k), said promoter and optionally said nucleic acid molecule encoding a transit peptide are encompassed in an expression cassette. Accordingly in an embodiment the invention provides a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

The invention further provides a vector comprising a nucleic acid molecule comprising 1) a nucleic acid molecule selected from the group consisting of:
(a) a nucleic acid molecule encoding a polypeptide shown in Table II, column 5 or 8, application no. 15, preferably shown in Table II A, application no. 15, in column 5, or in Table II A, application no. 15, column 8, or in Table II B, application no. 15, column 8, or a homolog or a fragment thereof;
(b) a nucleic acid molecule shown in Table I, application no. 15, column 5 or 8, preferably shown in Table I A, application no. 15, in column 5, or in Table I A, application no. 15, column 8, or in Table I B, application no. 15, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 15, preferably shown in Table II A, application no. 15, in column 5, or in Table II A, application no. 15, column 8, or in Table II B, application no. 15, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 15, preferably shown in Table I A, application no. 15, in column 5, or in Table I A, application no. 15, column 8, or in Table I B, application no. 15, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 15;
(i) a nucleic acid molecule encoding a polypeptide having the activity represented by a protein as depicted in column 5 of Table II, application no. 15;
(j) a nucleic acid molecule comprising a polynucleotide which is obtained by amplifying a cDNA library or a genomic library using the primers in Table III, application no. 15, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto;
   whereby, preferably, the nucleic acid molecule according to 1 (a), (b), (c), (d), (e), (f), (g), (h), (i), (j) or (k) distinguishes over the sequence depicted in Table I A and/or I B, application no. 15, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 15, column 5 or 8, or the coding region thereof, respectively. In another embodiment, the nucleic acid molecule of the present invention, preferably the coding region thereof, is 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 15, column 5 or 8, or the coding region thereof, respectively. In a further embodiment the nucleic acid molecule does not encode the polypeptide sequence shown in Table II A and/or II B, application no. 15, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 15, column 5 or 8 and does not encode a protein of the sequence shown in Table II A and/or II B, application no. 15, column 5 or 8.

Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 15, column 5 and 8, or the coding region thereof. In a further embodiment, the protein of the present invention is at least 30%,40%, 50%, 60%, 70%, 80%, 90%, 95% identical to protein sequence depicted in Table II A and/or II B, application no. 15, column 5 or 8 but less than 100%, preferably less than 99.999%, 99.99% or 99.9%, more preferably less than 99%, 985, 97%, 96% or 95% identical to the sequence shown in Table II A and/or II B, application no. 15, column 5 or 8.

(All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 15.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wildtype non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 15, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 15.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 15, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 15, in the same line as of the respective nucleic acid molecule according to 1) “plastidic” is depicted, or encoding a mitochondrial transit peptide, especially in case in column 6 of Table I, application no. 15, in the same line as of the respective nucleic acid molecule according to 1) “mitochondric” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 15, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 15, in the same line as of the respective nucleic acid molecule according to 1) “plastidic” is depicted, or encoding a mitochondrial transit peptide, especially in case in column 6 of Table I, application no. 15, in the same line as of the respective nucleic acid molecule according to 1) “mitochondric” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 15, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 15, in the same line as of the respective nucleic acid molecule according to 1) “plastidic” is depicted, or encoding a mitochondrial transit peptide, especially in case in column 6 of Table I, application no. 15, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 15.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 15, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 15.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 15, in the same line as of the respective nucleic acid molecule according to 1) “plastidic” is depicted, or encoding a mitochondrial transit peptide, especially in case in column 6 of Table I, application no. 15, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 15, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 15, in the same line as of the respective nucleic acid molecule according to 1) “plastidic” is depicted, or encoding a mitochondrial transit peptide, especially in case in column 6 of Table I, application no. 15, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 15 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 15, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 15 and confers a generation or an increase of the respective fine chemical in a non-human organism or a part thereof as compared to a corresponding wild-type non-human organism or part thereof.

[0181.1.7.15] to [0209.1.7.15] for the disclosure of these paragraphs see [0181.1.7.7] to [0209.1.7.7] above

The genes of the invention, coding in one embodiment for an activity selected from the group consisting of 2-C-methyl-D-erythritol 4-phosphate cytidylyltransferase, 2-isopropylmalate synthase, 2-oxoglutarate dehydrogenase E1 subunit, 30S ribosomal protein S3, 47266012-protein, 49747384_SOYBEAN-protein, 50S ribosomal protein L14, ABC transporter ATP binding component, ABC transporter permease protein, acetyl-CoA acetyltransferase, acetyl-CoA hydrolase, acetyltransferase, acyl-CoA dehydrogenase, acyl-CoA synthase, adenylate kinase, adenylylsulfate kinase, agmatinase, amino-acid acetyltransferase, aminotransferase, ammonium transporter, arginine N-succinyltransferase, aspartase, Atl g13930-protein, ATP-binding component of a transport system, auxin response factor, AX653549-protein, AY087308-protein, b0801-protein, b1003-protein, b1285-protein, b1425-protein, b1445-protein, b1522-protein, b1670-protein, b1672-protein, b1742-protein, b1898-protein, b2399-protein, b3777-protein, b3791-protein, b3814-protein, b3817-protein, b3989-protein, b4029-protein, b4050-protein, b4056-protein, beta-hydroxylase, betaine aldehyde dehydrogenase, bifunctional purine biosynthesis protein, branched chain aminotransferase, calcium-dependent protein kinase, cation-transporting ATPase, CBL-interacting protein kinase, CDP-diacylglycerol-glycerol-3-phosphate 3-phosphatidyltransferase, CDP-diacylglycerol-serine O-phosphatidyltransferase, CDP-glucose-4,6-dehydratase, citrate synthase, coproporphyrinogen III oxidase, CTP synthase, cystathionine beta-lyase, cysteine synthase A, cysteine synthase B, dATP pyrophosphohydrolase, dihydrolipoamide dehydrogenase, DnaJ-like chaperone, electron transfer flavoprotein subunit beta, electron transport complex protein, eukaryotic translation initiation factor 5, Fe—S subunit of oxidoreductase, flagellar M-ring protein, fumarate hydratase, gamma-glutamyltranspeptidase, gibberellin 20-oxidase, glucose dehydrogenase, glucose-1-phosphate cytidylyltransferase, glucose-6-phosphate 1-dehydrogenase, glutamate dehydrogenase, glutamate-ammonia-ligase, glutaredoxin, glycerate kinase, glycerol kinase, Glycine cleavage system T aminomethyltransferase, GM02LC17485-protein, GM02LC5744-protein, heat shock protein, heat shock transcription factor, hexose transporter, hydrolase, isochorismate synthase, isocitrate dehydrogenase, isocitrate lyase, leucyl/phenylalanyl-tRNA-protein transferase, lipopolysaccharide core biosynthesis protein, lipoprotein precursor, lysine decarboxylase, malate dehydrogenase, malonyl CoA-acyl carrier protein transacylase, membrane protein, membrane transport protein, methylglyoxal synthase, methylglyoxalase, methyltransferase, monothiol glutaredoxin, monthiol glutaredoxin, multiple antibiotic resistance protein, murein transglycosylase, NADH-quinone oxidoreductase subunit, O-antigen chain length determinant, oxidoreductase, oxidoreductase subunit, oxireductase, phenylacetic acid degradation protein, phenylalanine ammonialyase, phosphatase, phosphate import ATP-binding protein, photosystem I reaction centre subunit, photosystem II protein, polygalacturonase, protease, protein phosphatase, purinenucleoside phosphorylase, pyruvate carboxylase, pyruvate kinase, recombinase A, ribulokinase, RNA polymerase sigma factor, sec-independent protein translocase, Sec-independent protein translocase subunit, slr1170-protein, thioredoxin, thioredoxin family protein, thioredoxin reductase, threonine efflux protein, threonine synthase, transcription factor, transcriptional regulator, transcriptional regulator protein, translation initiation factor subunit, TTC1386-protein, xanthine dehydrogenase, XM_{—}473199-protein, ycr102c-protein, YDL159W-A-protein, ygl237c-protein, YGR193C-protein, ygr221 c-protein, yhr131c-protein, yhr207c-protein, YKL038W-protein, YKL140W-protein, ylr065c-protein, ylr326w-protein, yml084w-protein, yol073c-protein, yol160w-protein, zinc finger protein, and zinc transporter are also called “FCRP genes”.

[0211.1.7.15] to [0225.1.7.15] for the disclosure of these paragraphs see [0211.1.7.7] to [0225.1.7.7] above

In addition to the sequence mentioned in Table I, application no. 15, column 5 or 8, preferably the coding region thereof, or its homologs or fragments, it may be advantageous additionally to express and/or mutate further genes in the non-human organisms. Especially advantageously, additionally at least one further gene of the biosynthetic pathway of the fine chemical is expressed in the non-human organisms such as plants or microorganisms. It is also possible that the regulation of the natural genes has been modified advantageously so that the gene and/or its gene product is no longer subject to the regulatory mechanisms which exist in the non-human organisms. This leads to an increased synthesis of the respective amino acid since, for example, feedback regulations no longer exist to the same extent or not at all. In addition it might be advantageously to combine the nucleic acids sequences of the invention containing the sequences shown in the respective line in Table I, application no. 15, column 5 or 8, preferably the coding region thereof, or homologs or fragments thereof, with genes which generally support or enhance the growth or yield of the target non-human organisms, for example genes which lead to faster growth rate of non human organism like microorganisms or plants or genes which produce stress-, pathogen-, or herbicide-resistant plants.

In a further embodiment of the process of the invention, non-human organisms are grown, in which there is in addition to the expression of the nucleic acid molecule according to the invention simultaneous expression of at least one nucleic acid molecule or one of the genes which code for proteins involved in the citric acid cyle or vitamin c metabolism, in particular in genes of the amino acid metabolism, of glycolysis, of the tricarboxylic acid metabolism or their combinations. in the cytosol or in an organelle, like plastids or mitochondria.

[0228.1.7.15] to [0239.1.7.15] for the disclosure of these paragraphs see [0228.1.7.7] to [0239.1.7.7] above

In an embodiment of the present invention the nucleic acid construct of the present invention comprises a nucleic acid molecule as depicted in the respective line in Table I, application no. 15, column 5 or 8, preferably the coding region thereof, a fragment or a homolog thereof, and a plant promoter, such as the Big 35S promoter, the DC3 promoter, the PC Ubi promoter, the LegB4-promoter, the Super promoter, the USP promoter or the like. In another embodiment the nucleic acid construct of the present invention comprises a nucleic acid molecule as depicted in the respective line in Table I, application no. 15, column 5 or 8, preferably the coding region thereof, a fragment or a homolog thereof, and a plant promoter such as, for example, the USP, the LegB4-, the DC3 promoter or the ubiquitin promoter from parsley or other herein mentioned promoters. In a further embodiment the nucleic acid construct of the present invention comprises a nucleic acid molecule as depicted in the respective line in Table I, application no. 15, column 5 or 8, preferably the coding region thereof, a fragment or a homolog thereof, and a plant promoter, such as the Big 35S promoter, the PC Ubi promoter, the Super promoter, the USP promoter or the like. In addition different terminators may advantageously be used in these nucleic acid constructs; examples for transcriptional termination are polyadenylation signals.

[0241.1.7.15] to [0245.1.7.15] for the disclosure of these paragraphs see [0241.1.7.7] to [0245.1.7.7] above

Other preferred sequences for use in operable linkage in gene expression constructs are targeting sequences, which are required for targeting the gene product into specific cell compartments (for a review, see Kermode, Crit. Rev. Plant Sci. 15 (4), 285 (1996) and references cited therein), for example into the vacuole, the nucleus, all types of plastids, such as amyloplasts, chloroplasts, chromoplasts, the extracellular space, the mitochondria, the endoplasmic reticulum, elaioplasts, peroxisomes, glycosomes, and other compartments of cells or extracellular; preferred are sequences, which are involved in targeting to plastids as mentioned above. Sequences, which must be mentioned in this context are, in particular, the signal-peptide- or transit-peptide-encoding sequences which are known per se. For example, plastidtransit-peptide-encoding sequences enable the targeting of the expression product into the plastids of a plant cell. Targeting sequences are also known for eukaryotic and to a lower extent for prokaryotic organisms and can advantageously be operable linked with the nucleic acid molecule of the present invention as shown in Table I, application no. 15, columns 5 and 8, preferably the coding region thereof, or homologs or fragments thereof, and described herein to achieve an expression in one of said compartments or extracellular.

[0247.1.7.15] to [0249.1.7.15] for the disclosure of these paragraphs see [0247.1.7.7] to [0249.1.7.7] above

Preferred promoters are in particular those which bring about gene expression in tissues and organs, such as leaves, roots, seeds, pollen or xylem. Examples of tissue preferred or organ preferred promoters include, but are not limited to fruit-preferred, ovule-preferred, male tissue-preferred, seed-preferred, pollen-preferred integument-preferred, tuber-preferred, stalk-preferred, pericarp-preferred, leaf-preferred, stigma-preferred, pollen-preferred, anther-preferred, petal-preferred, sepal-preferred, pedicel-preferred, silique-preferred, stem-preferred, root-preferred promoters, and the like. Especially those promoters are preferred which bring about gene expression in tissues and organs in which the biosynthesis of at least one substrate or product of the citric acid cycle, a derivate of one of the substrates or products (for example pyruvate and malate as derivates of citramalate, acetyl-CoA as derivate of pyruvate or citrate, pyruvate as derivate of glycerate) or vitamin c, ascorbic acid or ascorbate and derivates like oxalate and/or threonic acidtakes place, like in seed cells, such as endosperm cells and cells of the developing embryo. Seed promoters are preferentially expressed during seed development and/or germination. For example, seed preferred promoters can be embryopreferred, endosperm preferred and seed coat-preferred (see Thompson et al., BioEssays 10, 108 (1989)). Examples of seed preferred promoters include, but are not limited to, cellulose synthase (celA), Cim1, gamma-zein, globulin-1, maize 19 kD zein (cZ19B1), and the like. Other suitable promoters are the oilseed rape napin gene promoter (U.S. Pat. No. 5,608,152), the Vicia faba USP promoter (Baeumlein et al., Mol Gen Genet, 225 (3), 459 (1991)), the Arabidopsis oleosin promoter (WO 98/45461), the Phaseolus vulgaris phaseolin promoter (U.S. Pat. No. 5,504,200), the Brassica Bce4 promoter (WO 91/13980), the bean arcs promoter, the carrot DcG3 promoter, or the Legumin B4 promoter (LeB4) (Baeumlein et al., Plant Journal, 2 (2), 233 (1992)), and promoters which bring about the seed-specific expression in monocotyledonous plants such as maize, barley, wheat, rye, rice and the like. Advantageous seed-specific promoters are the sucrose binding protein promoter (WO 00/26388), the phaseolin promoter and the napin promoter. Suitable promoters which must be considered are the barley Ipt2 or Ipt1 gene promoter (WO 95/15389 and WO 95/23230), and the promoters described in WO 99/16890 (promoters from the barley hordein gene, the rice glutelin gene, the rice oryzin gene, the rice prolamin gene, the wheat gliadin gene, the wheat glutelin gene, the maize zein gene, the oat glutelin gene, the sorghum kasirin gene and the rye secalin gene). Further suitable promoters are Amy32b, Amy 6-6 and Aleurain (U.S. Pat. No. 5,677,474), Bce4 (oilseed rape) (U.S. Pat. No. 5,530,149), glycinin (soya) (EP 571 741], phosphoenolpyruvate carboxylase (soya) (JP 06/62870), ADR12-2 (soya) (WO 98/08962), isocitrate lyase (oilseed rape) (U.S. Pat. No. 5,689,040]) or α-amylase (barley) (EP 781 8499. Other promoters which are available for the expression of genes in plants are leaf-specific promoters such as those described in DE-A 19 644 478 or light-regulated promoters such as, for example, the pea petE promoter.

[0251.1.7.15] to [0266.1.7.15] for the disclosure of these paragraphs see [0251.1.7.7] to [0266.1.7.7] above

An embodiment of the invention relates to a vector where the nucleic acid molecule according to the invention is linked operably to regulatory sequences which permit the expression in a prokaryotic or eukaryotic host. A further embodiment of the invention relates to a vector in which a nucleic acid sequence encoding one of the polypeptides shown in the respective line in Table II, application no. 15, columns 5 or 8, or homologs or fragments thereof, is functionally linked to a targeting sequence such as a plastidial or mitochondrial targeting sequence. A further embodiment of the invention relates to a vector in which a nucleic acid sequence encoding one of the polypeptides shown in the respective line in Table II, application no. 15, columns 5 or 8, or homologs or fragments thereof, is functionally linked to (a) regulatory sequence which permit the expression in an organelle, such as plastids or mitochondria, preferably plastids.

A further embodiment of the invention relates to a vector which comprises a nucleic acid sequence encoding one of the polypeptides shown in the respective line in Table II, application no. 15, columns 5 or 8, or homologs or fragments thereof, but no additional targeting sequence.

[0268.1.7.15] to [0273.1.7.15] for the disclosure of these paragraphs see [0268.1.7.7] to [0273.1.7.7] above

A further object of the invention relates to the use of a nucleic acid construct, e.g. an expression cassette, comprising one or more nucleic acid molecules encoding one or more polypeptides as depicted in the respective line(s) in Table II, application no. 15, columns 5 or 8, or homologs or fragments thereof, or comprising one or more nucleic acid molecules as depicted in the respective line(s) in Table I, application no. 15, columns 5 or 8, preferably the coding region thereof, or homologs or fragments thereof, or comprising a nucleic acid molecule hybridizing therewith for the transformation of a microorganism or a plant cell, plant tissues or a part of a plant

In doing so, depending on the choice of promoter, the nucleic acid molecules shown in the respective line in Table I, application no. 15, column 5 or 8, preferably the coding region thereof, or homologs or fragments thereof, or the nucleic acid molecules encoding a polypeptide as depicted in the respective line in Table II, application no. 15, column 5 or 8, or homologs or fragments thereof, can be expressed in the whole plant or specifically e.g. in the leaves, in the seeds, the nodules, in roots, in the stem or other parts of the plant. Those transgenic plants overproducing sequences, e.g. as depicted in the respective line in Table I, application no. 15, column 5 or 8, preferably the coding region thereof, or homologs or fragments thereof, the plant cell, plant tissues, plants, the reproductive material thereof, or parts thereof are a further object of the present invention.

Within the framework of the present invention, the production of the citramalate, citrate, fumarate, glycerate, malate, oxalate, pyruvate, succinate, or vitamin c and/or threonic acid is due to the generation or over-expression of one or more polypeptides as depicted in the respective line(s) in Table II, application no. 15, column 5 or 8, or homologs or fragments thereof, or encoded by the corresponding nucleic acid molecules as depicted in the respective line(s) in Table I, application no. 15, column 5 or 8, preferably the coding region thereof, or homologs or fragments thereof, in the non-human organism according to the invention, advantageously in the transgenic microorganism or plant according to the invention, at least for the duration of at least one plant generation.

A constitutive expression of the polypeptide as depicted in the respective line in Table II, application no. 15, column 5 or 8, or homologs or fragments thereof, or encoded by the respective nucleic acid molecule as depicted in the respective line in Table I, application no. 15, column 5 or 8, preferably the coding region thereof, or homologs or fragments thereof, may be advantageous. On the other hand, an inducible expression may also appear desirable. Expression of the polypeptide sequences of the invention can be either directed to the cytosol or to the organelles, such as plastids or mitochondria, preferably the plastids of the host cells, preferably the plant cells, or non-targeted.

for the disclosure of this paragraph see [0278.1.7.7] above

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no.

15, column 5 or 8, preferably shown in Table II A, application no. 15, column 5, or in Table II A, application no. 15, column 8, or in Table II B, application no. 15, column 8, or a homolog or a fragment thereof;
(b) a nucleic acid molecule shown in Table I, application no. 15, column 5 or 8, preferably shown in Table I A, application no. 15, column 5, or in Table I A, application no. 15, column 8, or in Table I B, application no. 15, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 15, preferably shown in Table II A, application no. 15, column 5, or in Table II A, application no. 15, column 8, or in Table II B, application no. 15, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nuoleic acid molecule sequence of a polynucleotide comprising the nucleic acid molecule shown in column 5 or 8 of Table I, application no. 15, preferably shown in Table I A, application no. 15, column 5, or in Table I A, application no. 15, column 8, or in Table I B, application no. 15, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 15;
(i) a nucleic acid molecule encoding a polypeptide having the activity represented by a respective protein as depicted in column 5 of Table II, application no. 15;
(j) a nucleic acid molecule comprising a polynucleotide which is obtained by amplifying a cDNA library or a genomic library using the primers in Table III, application no. 15, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
or a nucleic acid molecule comprising a sequence which is complementary thereto;
   and, if desired
2) nucleic acid molecule encoding a transit peptide, preferably encoding a plastidal transit peptide or a mitochondrial transit peptide;
   which are operable linked;]
   or comprising an expression cassette comprising
[0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 15, column 5 or 8, preferably shown in Table II A, application no. 15, column 5, or in Table II A, application no. 15, column 8, or in Table II B, application no. 15, column 8, or a homolog or a fragment thereof;
   (b) a nucleic acid molecule shown in Table I, application no. 15, column 5 or 8, preferably shown in Table I A, application no. 15, column 5, or in Table I A, application no. 15, column 8, or in Table I B, application no. 15, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 15, preferably shown in Table II A, application no. 15, column 5, or in Table II A, application no. 15, column 8, or in Table II B, application no. 15, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 15, preferably shown in Table I A, application no. 15, column 5, or in Table I A, application no. 15, column 8, or in Table I B, application no. 15, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 15;
   (i) a nucleic acid molecule encoding a polypeptide having the activity represented by a respective protein as depicted in column 5 of Table II, application no. 15;
   (j) a nucleic acid molecule comprising a polynucleotide which is obtained by amplifying a cDNA library or a genomic library using the primers in Table III, application no. 15, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising or being transformed by a vector comprising
[1) a nucleic acid molecule selected from the group consisting of
   (a) a nucleic acid molecule encoding a polypeptide shown in Table II, application no. 15, column 5 or 8, preferably shown in Table II A, application no. 15, column 5, or in Table II A, application no. 15, column 8, or in Table II B, application no. 15, column 8, or a homolog or a fragment thereof;
   (b) a nucleic acid molecule shown in Table I, application no. 15, column 5 or 8, preferably shown in Table I A, application no. 15, column 5, or in Table I A, application no. 15, column 8, or in Table I B, application no. 15, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 15, preferably shown in Table II A, application no. 15, column 5, or in Table II A, application no. 15, column 8, or in Table II B, application no. 15, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 15, preferably shown in Table I A, application no. 15, column 5, or in Table I A, application no. 15, column 8, or in Table I B, application no. 15, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 15;
   (i) a nucleic acid molecule encoding a polypeptide having the activity represented by a respective protein as depicted in column 5 of Table II, application no. 15;
   (j) a nucleic acid molecule comprising a polynucleotide which is obtained by amplifying a cDNA library or a genomic library using the primers in Table III, application no. 15, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]

as well as respective transgenic cells, tissue, parts of such non-human organism, e.g. plant cells, plant tissue, part of plants, like leaves, roots, stems, blossoms, seeds, fruits, pollen and the like.

(All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 15.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 15 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 15, in the same line as of the respective nucleic acid molecule according to 1) “plastidic” is depicted, or encoding a mitochondrial transit peptide, especially in case in column 6 of Table I, application no. 15, in the same line as of the respective nucleic acid molecule according to 1) “mitochondric” is depicted.

In another embodiment of said transgenic non-human organism the expression cassette does not comprise 2) a nucleic acid molecule encoding a transit peptide, especially in case in column 6 of Table I, application no. 15, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 15.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 15, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 15, in the same line as of the respective nucleic acid molecule according to 1) “plastidic” is depicted, or encoding a mitochondrial transit peptide, especially in case in column 6 of Table I, application no. 15, in the same line as of the respective nucleic acid molecule according to 1) “mitochondric” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 15, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 15, in the same line as of the respective nucleic acid molecule according to 1) “plastidic” is depicted, or encoding a mitochondrial transit peptide, especially in case in column 6 of Table I, application no. 15, in the same line as of the respective nucleic acid molecule according to 1) “mitochondric” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 15, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 15, in the same line as of the respective nucleic acid molecule according to 1) “plastidic” is depicted, or encoding a mitochondrial transit peptide, especially in case in column 6 of Table I, application no. 15, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 15.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 15, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 15.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 15, in the same line as of the respective nucleic acid molecule according to 1) “plastidic” is depicted, or encoding a mitochondrial transit peptide, especially in case in column 6 of Table I, application no. 15, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 15, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 15, in the same line as of the respective nucleic acid molecule according to 1) “plastidic” is depicted, or encoding a mitochondrial transit peptide, especially in case in column 6 of Table I, application no. 15, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 15 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 15, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 15 (in the same line) and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 15, column 5 or 8, preferably shown in Table II A, application no. 15, column 5, or in Table II A, application no. 15, column 8, or in Table II B, application no. 15, column 8, or a homolog or a fragment thereof;
   (b) a nucleic acid molecule shown in Table I, application no. 15, column 5 or 8, preferably shown in Table I A, application no. 15, column 5, or in Table I A, application no. 15, column 8, or in Table I B, application no. 15, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 15, preferably shown in Table II A, application no. 15, column 5, or in Table II A, application no. 15, column 8, or in Table II B, application no. 15, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 15, preferably shown in Table I A, application no. 15, column 5, or in Table I A, application no. 15, column 8, or in Table I B, application no. 15, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 15;
   (i) a nucleic acid molecule encoding a polypeptide having the activity represented by a respective protein as depicted in column 5 of Table II, application no. 15;
   (j) a nucleic acid molecule comprising a polynucleotide which is obtained by amplifying a cDNA library or a genomic library using the primers in Table III, application no. 15, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising an expression cassette comprising
[0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 15, column 5 or 8, preferably shown in Table II A, application no. 15, column 5, or in Table II A, application no. 15, column 8, or in Table II B, application no. 15, column 8, or a homolog or a fragment thereof;
   (b) a nucleic acid molecule shown in Table I, application no. 15, column 5 or 8, preferably shown in Table I A, application no. 15, column 5, or in Table I A, application no. 15, column 8, or in Table I B, application no. 15, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 15, preferably shown in Table II A, application no. 15, column 5, or in Table II A, application no. 15, column 8, or in Table II B, application no. 15, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 15, preferably shown in Table I A, application no. 15, column 5, or in Table I A, application no. 15, column 8, or in Table I B, application no. 15, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 15;
   (i) a nucleic acid molecule encoding a polypeptide having the activity represented by a respective protein as depicted in column 5 of Table II, application no. 15;
   (j) a nucleic acid molecule comprising a polynucleotide which is obtained by amplifying a cDNA library or a genomic library using the primers in Table III, application no. 15, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      and, if desired
2) nucleic acid molecule encoding a transit peptide, preferably encoding a plastidal transit peptide or a mitochondrial transit peptide;
   which are operable linked;]
   or comprising or being transformed by a vector comprising
[1) a nucleic acid molecule selected from the group consisting of
   (a) a nucleic acid molecule encoding a polypeptide shown in Table II, application no. 15, column 5 or 8, preferably shown in Table II A, application no. 15, column 5, or in Table II A, application no. 15, column 8, or in Table II B, application no. 15, column 8, or a homolog or a fragment thereof;
   (b) a nucleic acid molecule shown in Table I, application no. 15, column 5 or 8, preferably shown in Table I A, application no. 15, column 5, or in Table I A, application no. 15, column 8, or in Table I B, application no. 15, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 15, preferably shown in Table II A, application no. 15, column 5, or in Table II A, application no. 15, column 8, or in Table II B, application no. 15, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 15, preferably shown in Table I A, application no. 15, column 5, or in Table I A, application no. 15, column 8, or in Table I B, application no. 15, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 15;
   (i) a nucleic acid molecule encoding a polypeptide having the activity represented by a respective protein as depicted in column 5 of Table II, application no. 15;
   (j) a nucleic acid molecule comprising a polynucleotide which is obtained by amplifying a cDNA library or a genomic library using the primers in Table III, application no. 15, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      whereby, preferably, the nucleic acid molecule according to 1 (a), (b), (c), (d), (e), (f), (g), (h), (i), (j) or (k) distinguishes over the sequence depicted in Table I A and/or I B, application no. 15, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 15, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 15, column 5 or 8, or the coding region thereof, respectively. In a further embodiment the nucleic acid molecule does not encode the polypeptide sequence shown in Table II A and/or II B, application no. 15, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 15, column 5 or 8 and does not encode a protein of the sequence shown in Table II A and/or II B, application no. 15, column 5 or 8. Accordingly, in one embodiment, the protein encoded by a sequence of a nucleic acid according to 1 (a), (b), (c), (d), (e), (f), (g), (h), (i), (j) or (k) does not consist of the sequence shown in Table I A and/or I B, application no. 15, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 15, column 5 or 8 but less than 100%, preferably less than 99.999%, 99.99%, 99.9%, 99%, 98%, 97%, 96% or 95% identical to the sequence shown in Table II A and/or II B, application no. 15, column 5 or 8;
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 15.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 15 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 15, in the same line as of the respective nucleic acid molecule according to 1) “plastidic” is depicted, or encoding a mitochondrial transit peptide, especially in case in column 6 of Table I, application no. 15, in the same line as of the respective nucleic acid molecule according to 1) “mitochondric” is depicted.

In another embodiment thereof the expression cassette does not comprise 2) a nucleic acid molecule encoding a transit peptide, especially in case in column 6 of Table I, application no. 15, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 15.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the same line in column 5 of Table II, application no. 15, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 15, in the same line as of the respective nucleic acid molecule according to 1) “plastidic” is depicted, or encoding a mitochondrial transit peptide, especially in case in column 6 of Table I, application no. 15, in the same line as of the respective nucleic acid molecule according to 1) “mitochondric” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 15, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 15, in the same line as of the respective nucleic acid molecule according to 1) “plastidic” is depicted, or encoding a mitochondrial transit peptide, especially in case in column 6 of Table I, application no. 15, in the same line as of the respective nucleic acid molecule according to 1) “mitochondric” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 15, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 15, in the same line as of the respective nucleic acid molecule according to 1) “plastidic” is depicted, or encoding a mitochondrial transit peptide, especially in case in column 6 of Table I, application no. 15, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 15.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 15, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 15.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 15, in the same line as of the respective nucleic acid molecule according to 1) “plastidic” is depicted, or encoding a mitochondrial transit peptide, especially in case in column 6 of Table I, application no. 15, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 15, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 15, in the same line as of the respective nucleic acid molecule according to 1) “plastidic” is depicted, or encoding a mitochondrial transit peptide, especially in case in column 6 of Table I, application no. 15, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 15 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi,

Super and USP; a nucleic acid molecule encoding a transit peptide, especially in case in column 6 of Table I, application no. 15, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 15 and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

[0291.1.7.15] to [0299.1.7.15] for the disclosure of these paragraphs see [0291.1.7.7] to [0299.1.7.7] above

The term “biological active portion” or “biological activity” means a polypeptide as depicted in the respective line in Table II, application no. 15, column 3 or a portion of said polypeptide which still has at least 10% or 20%, preferably 30%, 40%, 50% or 60%, especially preferably 70%, 75%, 80%, 90% or 95% of the enzymatic or biological activity of the natural or starting enzyme or protein.

[0301.1.7.15] to [0304.1.7.15] for the disclosure of these paragraphs see [0301.1.7.7] to [0304.1.7.7] above

Synthetic oligonucleotide primers for the amplification, e.g. as shown in the respective line in Table III, application no. 15, column 8, by means of polymerase chain reaction can be generated on the basis of a sequence shown herein, for example the sequence shown in the respective line in Table I, application no. 15, columns 5 or 8, or the sequences derived from Table II, application no. 15, columns 5 or 8, respectively.

Moreover, it is possible to identify a conserved region of a protein from various organism by carrying out protein sequence alignments with the polypeptide encoded by the nucleic acid molecules of the present invention, in particular with the sequences encoded by the nucleic acid molecule shown in the respective line in column 5 or 8 of Table I, application no. 15, from which conserved regions, and in turn, degenerate primers can be derived. Conserved regions are those, which show a very little variation in the amino acid in one particular position of several homologs from different origin. The consensus sequence and polypeptide motifs shown in the respective line in column 8 of Table IV, application no. 15, are derived from such alignments of homologous proteins.

In an embodiment of the present invention, in the method of the present invention the activity of a polypeptide comprising or consisting of a consensus sequence or at least one polypeptide motif shown in the respective line in Table IV, application no. 15, column 8, is increased. In another embodiment thereof, the method of the present invention relates to a polypeptide comprising or consisting of a consensus sequence shown in the respective line in Table IV, application no. 15, column 8 or comprising at least one polypeptide motif shown in the respective line in Table IV, application no. 15, column 8, whereby not more than 20, 15, 10, 9, 8, 7, 6, 5 4, 3, 2 or 1, or 0 of the amino acid positions indicated can be replaced by any amino acid. In another embodiment thereof, the method of the present invention relates to a polypeptide comprising or consisting of a consensus sequence shown in the respective line in Table IV, application no. 15, column 8 or comprising at least one polypeptide motif shown in the respective line in Table IV, application no. 15, column 8, whereby not more than 15%, 10%, 5%, 4%, 3%, 2% or 1%, or 0% of the amino acid positions defined as distinct specific amino acids are/is replaced by another amino acid. In another embodiment thereof, the method of the present invention relates to a polypeptide comprising or consisting of a consensus sequence shown in the respective line in Table IV, application no. 15, column 8 or comprising at least one polypeptide motif shown in the respective line in Table IV, application no. 15, column 8, whereby not more than 20, 15, 10, 9, 8, 7, 6, 5, 4, 3, 2 or 1, or 0 amino acids are inserted into a consensus sequence or polypeptide motif.

In an embodiment of the present invention, in the method of the present invention the activity of a polypeptide comprising or consisting of a consensus sequence or at least one polypeptide motif shown in the respective line in Table IV, application no. 15, column 8, is increased, whereby said polypeptide distinguishes over the sequence depicted in Table II, application no. 15, columns 5 or 8 by one or more amino acids.

In one embodiment, said polypeptide of the invention distinguishes over the sequence shown in the respective line in Table II, application no. 15, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II B, application no. 15, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in Table II, application no. 15, columns 5 or 8.

[0309.1.7.15] to [0321.1.7.15] for the disclosure of these paragraphs see [0309.1.7.7] to [0321.1.7.7.] above

Polypeptides having above-mentioned activity, i.e. conferring the production or the increased production of the fine chemical citramalate, citrate, fumarate, glycerate, malate, oxalate, pyruvate, succinate, or vitamin c and/or threonic acid as compared to a corresponding, e.g. non-transformed, wild type non-human organism, like a microorganism or a plant cell, plant or part thereof, can be encoded by other DNA sequences which hybridize to the sequences shown in the respective line in Table I, application no. 15, columns 5 and 8, preferably the coding region thereof, at least under relaxed hybridization conditions and which encode the expression of polypeptides conferring the production or the increased production of the respective fine chemical citramalate, citrate, fumarate, glycerate, malate, oxalate, pyruvate, succinate, or vitamin c and/or threonic acid as compared to a corresponding, e.g. non-transformed, wild type non-human organism, like a microorganism or a plant cell, plant or part thereof.

[0323.1.7.15] to [0329.1.7.15] for the disclosure of these paragraphs see [0323.1.7.7] to [0329.1.7.7] above

Further, the nucleic acid molecule of the invention comprises a nucleic acid molecule, which is a complement of one of the nucleotide sequences of above-mentioned nucleic acid molecules or a portion thereof. A nucleic acid molecule or its sequence which is complementary to one of the nucleotide molecules or sequences shown in the respective line in Table I, application no. 15, columns 5 or 8, preferably the coding region thereof, homologs or fragments thereof, is one which is sufficiently complementary to one of the nucleotide molecules or sequences shown in the respective line in Table I, application no. 15, columns 5 or 8, preferably the coding region thereof, homologs or fragments thereof, such that it can hybridize to one of the nucleotide sequences shown in Table I, application no. 15, columns 5 or 8, preferably the coding region thereof, homologs or fragments thereof, thereby forming a stable duplex. Preferably, the hybridization is performed under stringent hybridization conditions. However, a complement of one of the herein disclosed sequences is preferably a sequence complement thereto according to the base pairing of nucleic acid molecules well known to the skilled person. For example, the bases A and G undergo base pairing with the bases T and U or C, resp. and visa versa. Modifications of the bases can influence the base-pairing partner.

The nucleic acid molecule of the invention comprises a nucleotide sequence which is at least about 30%, 35%, 40%, 45%, 50%, 55%, 60%, 65%, 70%, 75%, 80%, 90%, 95%, 96%, 97%, 98%, 99%, 99.5% or more homologous to a nucleotide sequence shown in Table I, application no. 15, columns 5 or 8, preferably the coding region thereof, homologs or fragments thereof, and preferably has above-mentioned activity, in particular conferring the production or the increased production of the fine chemical citramalate, citrate, fumarate, glycerate, malate, oxalate, pyruvate, succinate, or vitamin c and/or threonic acid, respectively, after increasing the activity or an activity of a gene as shown in the respective line in Table I or of a gene product, e.g. as shown in the respective line in Table II, application no. 15, column 5 or 8, by for example in one embodiment expression either in the cytosol or in an organelle such as a plastid or mitochondria or both, preferably in a plastid, or in another embodiment non-targeted or targeted.

The nucleic acid molecule of the invention comprises a nucleotide sequence or molecule which hybridizes, preferably hybridizes under stringent conditions as defined herein, to one of the nucleic acid molecule shown in the respective line in Table I, preferably Table IB, application no. 15, columns 5 or 8, preferably the coding region thereof, fragments or homologs thereof, and encodes a protein having above-mentioned activity, e.g. conferring the production or the increased production of the respective fine chemical citramalate, citrate, fumarate, glycerate, malate, oxalate, pyruvate, succinate, or vitamin c and/or threonic acid as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted, and optionally, the activity thereof is selected from the group consisting of 2-C-methyl-D-erythritol 4-phosphate cytidylyltransferase, 2-isopropylmalate synthase, 2-oxoglutarate dehydrogenase E1 subunit, 30S ribosomal protein S3, 47266012-protein, 49747384_SOYBEAN-protein, 50S ribosomal protein L14, ABC transporter ATP binding component, ABC transporter permease protein, acetyl-CoA acetyltransferase, acetyl-CoA hydrolase, acetyltransferase, acyl-CoA dehydrogenase, acyl-CoA synthase, adenylate kinase, adenylylsulfate kinase, agmatinase, amino-acid acetyltransferase, aminotransferase, ammonium transporter, arginine N-succinyltransferase, aspartase, Atl g13930-protein, ATP-binding component of a transport system, auxin response factor, AX653549-protein, AY087308-protein, b0801-protein, b1003-protein, b1285-protein, b1425-protein, b1445-protein, b1522-protein, b1670-protein, b1672-protein, b1742-protein, b1898-protein, b2399-protein, b3777-protein, b3791-protein, b3814-protein, b3817-protein, b3989-protein, b4029-protein, b4050-protein, b4056-protein, beta-hydroxylase, betaine aldehyde dehydrogenase, bifunctional purine biosynthesis protein, branched chain aminotransferase, calcium-dependent protein kinase, cation-transporting ATPase, CBL-interacting protein kinase, CDP-diacylglycerol-glycerol-3-phosphate 3-phosphatidyltransferase, CDP-diacylglycerol-serine O-phosphatidyl-transferase, CDP-glucose-4,6-dehydratase, citrate synthase, coproporphyrinogen III oxidase, CTP synthase, cystathionine beta-lyase, cysteine synthase A, cysteine synthase B, dATP pyrophosphohydrolase, dihydrolipoamide dehydrogenase, DnaJ-like chaperone, electron transfer flavoprotein subunit beta, electron transport complex protein, eukaryotic translation initiation factor 5, Fe—S subunit of oxidoreductase, flagellar M-ring protein, fumarate hydratase, gamma-glutamyltranspeptidase, gibberellin 20-oxidase, glucose dehydrogenase, glucose-1-phosphate cytidylyltransferase, glucose-6-phosphate 1-dehydrogenase, glutamate dehydrogenase, glutamate-ammonia-ligase, glutaredoxin, glycerate kinase, glycerol kinase, Glycine cleavage system T aminomethyltransferase, GM02LC17485-protein, GM02LC5744-protein, heat shock protein, heat shock transcription factor, hexose transporter, hydrolase, isochorismate synthase, isocitrate dehydrogenase, isocitrate lyase, leucyl/phenylalanyl-tRNA-protein transferase, lipopolysaccharide core biosynthesis protein, lipoprotein precursor, lysine decarboxylase, malate dehydrogenase, malonyl CoA-acyl carrier protein transacylase, membrane protein, membrane transport protein, methylglyoxal synthase, methylglyoxalase, methyltransferase, monothiol glutaredoxin, monthiol glutaredoxin, multiple antibiotic resistance protein, murein transglycosylase, NADH-quinone oxidoreductase subunit, O-antigen chain length determinant, oxidoreductase, oxidoreductase subunit, oxireductase, phenylacetic acid degradation protein, phenylalanine ammonialyase, phosphatase, phosphate import ATP-binding protein, photosystem I reaction centre subunit, photosystem II protein, polygalacturonase, protease, protein phosphatase, purinenucleoside phosphorylase, pyruvate carboxylase, pyruvate kinase, recombinase A, ribulokinase, RNA polymerase sigma factor, sec-independent protein translocase, Sec-independent protein translocase subunit, slr1170-protein, thioredoxin, thioredoxin family protein, thioredoxin reductase, threonine efflux protein, threonine synthase, transcription factor, transcriptional regulator, transcriptional regulator protein, translation initiation factor subunit, TTC1386-protein, xanthine dehydrogenase, XM_{—}473199-protein, ycr102c-protein, YDL159W-A-protein, ygl237c-protein, YGR193C-protein, ygr221c-protein, yhr131c-protein, yhr207c-protein, YKL038W-protein, YKL140W-protein, ylr065c-protein, ylr326w-protein, yml084w-protein, yol073c-protein, yol160w-protein, zinc finger protein, and zinc transporter, respectively.

Moreover, the nucleic acid molecule of the invention can comprise only a porganism tion of the coding region of one of the nucleic acid molecules depicted in Table I, application no. 15, columns 5 or 8, for example a fragment which can be used as a probe or primer or a fragment encoding a biologically active portion of the polypeptide of the present invention or of a polypeptide used in the process of the present invention, i.e. having above-mentioned activity, e.g. conferring the production or the increased production of the respective fine chemical citramalate, citrate, fumarate, glycerate, malate, oxalate, pyruvate, succinate, or vitamin c and/or threonic acid as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted. The nucleotide sequences determined from the cloning of the present protein-according-to-the-invention-encoding gene allows for the generation of probes and primers designed for use in identifying and/or cloning its homologues from other cell types and organisms. The probe/primer typically comprises a substantially purified oligonucleotide. The oligonucleotide typically comprises a region of nucleotide sequence that hybridizes under stringent conditions to at least about 12, 15 preferably about 20 or 25, more preferably about 40, 50 or 75 consecutive nucleotides of a sense strand of one of the sequences set forth, e.g., in the respective line in Table I, application no. 15, column 5 or 8, preferably the coding region thereof, or a homolog or a fragment thereof, an anti-sense sequence of one of the sequences, e.g., set forth in the respective line in Table I, application no. 15, columns 5 or 8, preferably the coding region thereof, or a homolog or a fragment thereof or naturally occurring mutants thereof. Primers based on a nucleic acid molecule of invention can be used in PCR reactions to clone homologs of the polypeptide of the invention or of the polypeptide used in the process of the invention, e.g. as the primers described in the examples of the present invention, e.g. as shown in the examples. A PCR with the primers shown in the respective line in Table III, column 8 will result in a fragment of the gene product as shown in Table II, application no. 15, column 5.

Primer sets are interchangeable. The person skilled in the art knows how to design and combine said primers to result in the desired product, e.g. in a full length clone or a partial sequence. Probes based on the sequences of the nucleic acid molecule of the invention or used in the process of the present invention can be used to detect transcripts or genomic sequences encoding the same or homologous proteins. The probe can further comprise a label group attached thereto, e.g. the label group can be a radioisotope, a fluorescent compound, an enzyme, or an enzyme co-factor. Such probes can be used as a part of a genomic marker test kit for identifying cells which express a polypeptide of the invention or used in the process of the present invention, such as by measuring a level of an encoding nucleic acid molecule in a sample of cells, e.g. detecting mRNA levels or determining, whether a genomic gene comprising the sequence of the polynucleotide of the invention or used in the processes of the present invention has been mutated or deleted.

As the expression of a respective nucleic acids of the invention is related to the synthesis of the respective fine chemical citramalate, citrate, fumarate, glycerate, malate, oxalate, pyruvate, succinate, or vitamin c and/or threonic acid its function as a probe extends to the detection of microorganisms, plant tissues, plants, plant variets, plant ecotypes or plant genera with varying capability or potential for synthesis of the respective fine chemical citramalate, citrate, fumarate, glycerate, malate, oxalate, pyruvate, succinate, or vitamin c and/or threonic acid. Therefore in one embodiment the present invention relates to a method for analyzing the capability or potential of a plant tissue, a plant, a plant variety or plant ecotype to produce the fine chemical citramalate, citrate, fumarate, glycerate, malate, oxalate, pyruvate, succinate, or vitamin c and/or threonic acid by using the nucleic acid of the invention or parts thereof as a probe to detect the amount of the nucleic acid of the invention in the non-human organism or a part thereof in comparison to another non-human organism.

The nucleic acid molecule of the invention encodes a polypeptide or portion thereof which includes an amino acid sequence which is sufficiently homologous to the amino acid sequence shown in respective line in Table II, application no. 15, columns 5 or 8 such that the protein or portion thereof maintains the ability to confer the production or the increased production of the respective fine chemical citramalate, citrate, fumarate, glycerate, malate, oxalate, pyruvate, succinate, or vitamin c and/or threonic acid as compared to a corresponding, e.g. non-transformed, wild type non-human organism, like a microorganism or a plant cell, plant or part thereof, in particular increasing the activity as mentioned above or as described in the examples in microorganisms or plants.

As used herein, the language “sufficiently homologous” refers to proteins or portions thereof which have amino acid sequences which include a minimum number of identical or equivalent amino acids (e.g. an amino acid which has a similar side chain as an amino acid in one of the sequences of the polypeptide of the present invention) to an amino acid sequence shown in the respective line in Table II, application no. 15, columns 5 or 8 such that the protein or portion thereof is able to confer the production or the increased production of the respective fine chemical citramalate, citrate, fumarate, glycerate, malate, oxalate, pyruvate, succinate, or vitamin c and/or threonic acid as compared to a corresponding, e.g. non-transformed, wild type non-human organism, like a microorganism or a plant cell, plant or part thereof. For example having the activity of a protein as shown in the respective line in Table II, application no. 15, column 3 and as described herein.

In an embodiment of the present invention, the nucleic acid molecule of the present invention comprises a nucleic acid molecule that encodes a portion of the protein of the present invention. The protein is at least about 30%, 35%, 40%, 45% 50%, 55%, 60%, 65%, 70%, 75%, 80%, 85%, 90%, 91%, 92%, 93%, 94%, 95%, 96%, 97%, 98%, 99%, 99.5% or more homologous to an entire amino acid sequence shown in the respective line of Table II, application no. 15, column 5 or 8 and has the above-mentioned activity, e.g. conferring the production or the increased production of the respective fine chemical citramalate, citrate, fumarate, glycerate, malate, oxalate, pyruvate, succinate, or vitamin c and/or threonic acid as compared to a corresponding, e.g. non-transformed, wild type non-human organism, like a microorganism or a plant cell, plant or part thereof by, for example in a embodiment expression either in the cytosol or in an organelle such as a plastid or mitochondria or both, preferably in a plastid, or in another embodiment by targeted or non-targeted expression.

for the disclosure of this paragraph see [0338.1.7.7] above

for the disclosure of this paragraph see [0339.1.7.7] above

The invention further relates to nucleic acid molecules or methods using said nucleic acid molecules that differ from one of the nucleotide sequences shown in the respective line in Table I, application no. 15, columns 5 or 8, preferably the coding region thereof, or fragments or homologs thereof, due to degeneracy of the genetic code but encode a polypeptide of the present invention, in particular a polypeptide having the above-mentioned activity, e.g. as those polypeptides depicted in the respective line in Table II, application no. 15, columns 5 or 8, fragments or homologs thereof. Advantageously, the nucleic acid molecule of the invention comprises, or in an other embodiment has, a nucleotide sequence encoding a protein comprising, or in an other embodiment having, an amino acid sequence shown in the respective line in Table II, application no. 15, columns 5 or 8, fragments or homologs thereof. In a further embodiment, the nucleic acid molecule of the invention encodes a full length protein which is substantially homologous to an amino acid sequence shown in the respective line in Table II, application no. 15, columns 5 or 8, or homologs thereof. However, in an embodiment, the nucleic acid molecule of the present invention does not consist of the sequence shown in the respective line in Table I, preferably Table IA, application no. 15, columns 5 or 8, preferably the coding region thereof, but the functional homologs thereof.

[0341.1.7.15] to [0343.1.7.15] for the disclosure of these paragraphs see [0341.1.7.7] to [0343.1.7.7] above

Accordingly, in another embodiment, a nucleic acid molecule of the invention is at least 15, 20, 25 or 30 nucleotides in length. Preferably, it hybridizes under stringent conditions to a nucleic acid molecule comprising a nucleotide sequence of the nucleic acid molecule of the present invention or used in the process of the present invention, e.g. comprising the sequence shown in the respective line in Table I, application no. 15, columns 5 or 8, preferably the coding region thereof. In another embodiment the nucleic acid molecule is preferably at least 20, 30, 50, 100, 250 or more nucleotides in length.

for the disclosure of this paragraph see [0345.1.7.7] above

Preferably, a nucleic acid molecule of the invention that hybridizes under stringent conditions to a sequence shown in the respective line in Table I, application no. 15, columns 5 or 8, preferably the coding region thereof, corresponds to a naturally-occurring nucleic acid molecule of the invention. As used herein, a “naturally-occurring” nucleic acid molecule refers to a RNA or DNA molecule having a nucleotide sequence that occurs in nature (e.g. encodes a natural protein). Preferably, the nucleic acid molecule encodes a natural protein having above-mentioned activity, e.g. conferring the production or the increased production of the respective fine chemical citramalate, citrate, fumarate, glycerate, malate, oxalate, pyruvate, succinate, or vitamin c and/or threonic acid as compared to a corresponding, e.g. non-transformed, wild type non-human organism, like a microorganism or a plant cell, plant or part thereof after increasing the expression or activity thereof or the activity of a protein of the invention or used in the process of the invention, in an embodiment for example expression either in the cytosol or in an organelle such as a plastid or mitochondria, preferably in plastids, or, in another embodiment by targeted or non-targeted expression.

for the disclosure of this paragraph see [0347.1.7.7] above

For example, nucleotide substitutions leading to amino acid substitutions at “non-essential” amino acid residues can be made in a sequence of the nucleic acid molecule of the invention or used in the process of the invention, e.g. shown in the respective line in Table I, application no. 15, columns 5 or 8, preferably the coding region thereof, or fragments or homologs thereof.

for the disclosure of this paragraph see [0349.1.7.7] above

for the disclosure of this paragraph see [0350.1.7.7] above

Accordingly, the invention relates to nucleic acid molecules encoding a polypeptide having above-mentioned activity, in a non-human organisms or parts thereof by for example expression either in the cytosol or in an organelle such as a plastid or mitochondria or both, preferably in plastids, that contain changes in amino acid residues that are not essential for said activity. Such polypeptides differ in amino acid sequence from a sequence depicted in the respective line in Table II, especially Table IIA, application no. 15, columns 5 or 8, but retain said activity described herein. The nucleic acid molecule can comprise a nucleotide sequence encoding a polypeptide, wherein the polypeptide comprises an amino acid sequence at least about 50% identical to an amino acid sequence shown in the respective line in Table II, application no. 15, columns 5 or 8, and is capable of conferring the production or the increased production of the respective fine chemical citramalate, citrate, fumarate, glycerate, malate, oxalate, pyruvate, succinate, or vitamin c and/or threonic acid as compared to a corresponding, e.g. non-transformed, wild type non-human organism, like a microorganism or a plant cell, plant or part thereof, after increasing its activity for example in an embodiment by expression either in the cytosol or in an organelle such as a plastid or mitochondria or both, preferably in plastids, or, in another embodiment by targeted or non-targeted expression. Preferably, the protein encoded by the nucleic acid molecule is at least about 60%, 70%, 80%, 90%, 95%, 96%, 97%, 98%, 99% or 99.5% identical to the sequence shown in the respective line in Table II, application no. 15, columns 5 or 8.

[0352.1.7.15] to [0357.1.7.15] for the disclosure of these paragraphs see [0352.1.7.7] to [0357.1.7.7] above

Functional equivalents of the nucleic acid molecules according to the present invention and/or used in the process according to the present invention represent an embodiment of homologs. Functional equivalents derived from the nucleic acid molecules as shown in the respective line in Table I, application no. 15, columns 5 or 8, preferably the coding region thereof, according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the nucleic acid molecules as shown in the respective line in Table I, application no. 15, columns 5 or 8, preferably the coding region thereof, according to the invention and encode polypeptides having essentially the same properties as the polypeptide as shown in the respective line in Table II, application no. 15, columns 5 or 8.

Functional equivalents derived from one of the polypeptides as shown in the respective line in Table II, application no. 15, columns 5 or 8 according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the polypeptides as shown in the respective line in Table II, application no. 15, columns 5 or 8 according to the invention and having essentially the same properties as the polypeptide as shown in the respective line in Table II, application no. 15, columns 5 or 8.

for the disclosure of this paragraph see [0359.1.7.7] above

A nucleic acid molecule encoding a homolog to a polypeptide depicted in the respective line in Table II, application no. 15, columns 5 or 8 can be created by introducing one or more nucleotide substitutions, additions or deletions into the nucleic acid molecule of the present invention, in particular of Table I, application no. 15, columns 5 or 8, in the respective line, such that one or more amino acid substitutions, additions or deletions are introduced into the encoded protein. Mutations can be introduced into the nucleic acid molecules as depicted in the respective line in Table I, application no. 15, columns 5 or 8, preferably the coding region thereof, by standard techniques, such as site-directed mutagenesis, PCR-mediated mutagenesis or other methods known to the person skilled in the art.

[0361.1.7.15] to [0363.1.7.15] for the disclosure of these paragraphs see [0361.1.7.7] to [0363.1.7.7] above

Homologues of the nucleic acid sequences used, with the sequences shown in the respective line in Table I, application no. 15, columns 5 or 8, preferably the coding region thereof, comprise also allelic variants with at least approximately 30%, 35%, 40%, 45%, 50%, 55%, 60%, 65%, 70%, 75%, 80%, 85%, 90%, 91%, 92%, 93%, 94% 95%. 96%, 97%, 98%, 99%, 99.5% or more % homology with one of the nucleotide sequences shown or the abovementioned derived nucleic acid sequences or their homologues or derivatives or parts of these. Allelic variants encompass in particular functional variants which can be obtained by deletion, insertion or substitution of nucleotides from the sequences shown in the respective line, preferably in Table I, columns 5 or 8, preferably the coding region thereof, or from the derived nucleic acid sequences, the intention being, however, that the enzymatic activity or the biological activity of the resulting proteins synthesized is advantageously retained or increased.

In one embodiment of the present invention, the nucleic acid molecule of the invention or used in the process of the invention comprises the sequences shown in the respective line in any of the Table I, application no. 15, columns 5 or 8, preferably the coding region thereof, fragments or homologs thereof. It is preferred that the nucleic acid molecule comprises as little as possible other nucleotides not shown in the respective line in any one of Table I, application no. 15, columns 5 or 8, preferably the coding region thereof, fragments or homologs thereof. In one embodiment, the nucleic acid molecule comprises less than 500, 400, 300, 200, 100, 90, 80, 70, 60, 50 or 40 further nucleotides. In a further embodiment, the nucleic acid molecule comprises less than 30, 20 or 10 further nucleotides. In one embodiment, the nucleic acid molecule used in the process of the invention is identical to the sequences shown in the respective line in Table I, application no. 15, columns 5 or 8, preferably the coding region thereof.

Also preferred is that the nucleic acid molecule used in the process of the invention encodes a polypeptide comprising the sequence shown in the respective line in Table II, application no. 15, columns 5 or 8, fragments or homologs thereof. In one embodiment, the nucleic acid molecule encodes less than 150, 130, 100, 80, 60, 50, 40 or 30 further amino acids. In a further embodiment, the encoded polypeptide comprises less than 20, 15, 10, 9, 8, 7, 6 or 5 further amino acids. In one embodiment used in the inventive process, the encoded polypeptide is identical to the sequences shown in the respective line in Table II, application no. 15, columns 5 or 8.

In one embodiment, the nucleic acid molecule of the invention or used in the process encodes a polypeptide comprising the sequence shown in the respective line in Table II, application no. 15, columns 5 or 8, or fragments or homologs thereof, and comprises less than 100 further nucleotides. In a further embodiment, said nucleic acid molecule comprises less than 30 further nucleotides. In one embodiment, the nucleic acid molecule used in the process is identical to a coding sequence of the sequences shown in the respective line in Table I, application no. 15, columns 5 or 8.

Polypeptides (=proteins), which still have the essential biological or enzymatic activity of the polypeptide of the present invention conferring the production or the increased production of the respective fine chemical citramalate, citrate, fumarate, glycerate, malate, oxalate, pyruvate, succinate, or vitamin c and/or threonic acid as compared to a corresponding, e.g. non-transformed, wild type non-human organism, like a microorganism or a plant cell, plant or part thereof, i.e. whose activity is essentially not reduced, are polypeptides with at least 10%, 20%, 30%, 40%, 50%, 60%, 70%, 80%, 90%, 95% or more of the wild type biological activity or enzymatic activity, advantageously, the activity is essentially not reduced in comparison with the activity of a polypeptide shown in the respective line in Table II, application no. 15, columns 5 or 8 expressed under identical conditions.

Homologues of nucleic acid molecules shown in the respective line in Table I, application no. 15, columns 5 or 8, preferably the coding region thereof, or fragments thereof, or of the derived sequences shown in the respective line in Table II, application no. 15, columns 5 or 8, or fragments thereof, also mean truncated sequences, cDNA, single-stranded DNA or RNA of the coding and noncoding DNA sequence. Homologues of said sequences are also understood as meaning derivatives, which comprise noncoding regions such as, for example, UTRs, introns, terminators, enhancers or promoter variants. The promoters upstream of the nucleotide sequences stated can be modified by one or more nucleotide substitution(s), insertion(s) and/or deletion(s) without, however, interfering with the functionality or activity either of the promoters, the open reading frame (=ORF) or with the 3′-regulatory region such as terminators or other 3′-regulatory regions, which are far away from the ORF. It is furthermore possible that the activity of the promoters is increased by modification of their sequence, or that they are replaced completely by more active promoters, even promoters from heterologous organisms. Appropriate promoters are known to the person skilled in the art and are mentioned herein below.

[0370.1.7.15] to [0379.1.7.15] for the disclosure of theses paragraphs see [0370.1.7.7] to [0379.1.7.7] above

Moreover, a native polypeptide conferring the increase of the respective fine chemical citramalate, citrate, fumarate, glycerate, malate, oxalate, pyruvate, succinate, or vitamin c and/or threonic acid in a non-human organism or a part thereof can be isolated from cells (e.g., endothelial cells), for example using the antibody of the present invention as described below, in particular, an antibody against proteins having 2-C-methyl-D-erythritol 4-phosphate cytidylyltransferase, 2-isopropylmalate synthase, 2-oxoglutarate dehydrogenase E1 subunit, 30S ribosomal protein S3, 47266012-protein, 49747384_SOYBEAN-protein, 50S ribosomal protein L14, ABC transporter ATP binding component, ABC transporter permease protein, acetyl-CoA acetyltransferase, acetyl-CoA hydrolase, acetyltransferase, acyl-CoA dehydrogenase, acyl-CoA synthase, adenylate kinase, adenylylsulfate kinase, agmatinase, amino-acid acetyltransferase, aminotransferase, ammonium transporter, arginine N-succinyltransferase, aspartase, At1g13930-protein, ATP-binding component of a transport system, auxin response factor, AX653549-protein, AY087308-protein, b0801-protein, b1003-protein, b1285-protein, b1425-protein, b1445-protein, b1522-protein, b1670-protein, b1672-protein, b1742-protein, b1898-protein, b2399-protein, b3777-protein, b3791-protein, b3814-protein, b3817-protein, b3989-protein, b4029-protein, b4050-protein, b4056-protein, beta-hydroxylase, betaine aldehyde dehydrogenase, bifunctional purine biosynthesis protein, branched chain aminotransferase, calcium-dependent protein kinase, cation-transporting ATPase, CBL-interacting protein kinase, CDP-diacylglycerol-glycerol-3-phosphate 3-phosphatidyltransferase, CDP-d iacylglycerolserine O-phosphatidyltransferase, CDP-glucose-4,6-dehydratase, citrate synthase, coproporganism phyrinogen III oxidase, CTP synthase, cystathionine beta-lyase, cysteine synthase A, cysteine synthase B, dATP pyrophosphohydrolase, dihydrolipoamide dehydrogenase, DnaJ-like chaperone, electron transfer flavoprotein subunit beta, electron transport complex protein, eukaryotic translation initiation factor 5, Fe—S subunit of oxidoreductase, flagellar M-ring protein, fumarate hydratase, gamma-glutamyltranspeptidase, gibberellin 20-oxidase, glucose dehydrogenase, glucose-1-phosphate cytidylyltransferase, glucose-6-phosphate 1-dehydrogenase, glutamate dehydrogenase, glutamate-ammonia-ligase, glutaredoxin, glycerate kinase, glycerol kinase, Glycine cleavage system T aminomethyltransferase, GM02LC17485-protein, GM02LC5744-protein, heat shock protein, heat shock transcription factor, hexose transporter, hydrolase, isochorismate synthase, isocitrate dehydrogenase, isocitrate lyase, leucyl/phenylalanyl-tRNA-protein transferase, lipopolysaccharide core biosynthesis protein, lipoprotein precursor, lysine decarboxylase, malate dehydrogenase, malonyl CoA-acyl carrier protein transacylase, membrane protein, membrane transport protein, methylglyoxal synthase, methylglyoxalase, methyltransferase, monothiol glutaredoxin, monthiol glutaredoxin, multiple antibiotic resistance protein, murein transglycosylase, NADH-quinone oxidoreductase subunit, O-antigen chain length determinant, oxidoreductase, oxidoreductase subunit, oxireductase, phenylacetic acid degradation protein, phenylalanine ammonia-lyase, phosphatase, phosphate import ATP-binding protein, photosystem I reaction centre subunit, photosystem II protein, polygalacturonase, protease, protein phosphatase, purine-nucleoside phosphorylase, pyruvate carboxylase, pyruvate kinase, recombinase A, ribulokinase, RNA polymerase sigma factor, sec-independent protein translocase, Sec-independent protein translocase subunit, slr1170-protein, thioredoxin, thioredoxin family protein, thioredoxin reductase, threonine efflux protein, threonine synthase, transcription factor, transcriptional regulator, transcriptional regulator protein, translation initiation factor subunit, TTC1386-protein, xanthine dehydrogenase, XM_{—}473199-protein, ycr102c-protein, YDL159W-A-protein, ygl237c-protein, YGR193C-protein, ygr221c-protein, yhr131c-protein, yhr207c-protein, YKL038W-protein, YKL140W-protein, ylr065c-protein, ylr326w-protein, yml084w-protein, yol073c-protein, yol160w-protein, zinc finger protein, or zinc transporter activity, respectively, or an antibody against polypeptides as shown in the respective line in Table II, application no. 15, columns 5 or 8, or fragments or homologs thereof which can be produced by standard techniques utilizing the polypeptid of the present invention or fragment thereof, i.e., the polypeptide of this invention (FCRP). Preferred are monoclonal antibodies.

for the disclosure of this paragraph see [0381.1.7.7] above

In one embodiment, the present invention relates to a polypeptide as depicted in the respective line in Table II, especially in Table IIA or especially in Table IIB, application no. 15, columns 5 or 8, or fragment or homolog thereof or as coded by the nucleic acid molecule shown in the respective line in Table I, especially Table IA or especially Table IB, application no. 15, columns 5 or 8, preferably the coding region thereof, or fragment or homolog thereof.

In an embodiment, the present invention relates to a polypeptide comprising or consisting of a consensus sequence shown in the respective line in Table IV, application no. 15, column 8 or at least one polypeptide motif shown in the respective line in Table IV, application no. 15, column 8.

In an embodiment thereof, the present invention relates to a polypeptide comprising or consisting of a consensus sequence shown in the respective line in Table IV, application no. 15, column 8 or comprising at least one polypeptide motif shown in the respective line in Table IV, application no. 15, column 8, whereby not more than 20, 15, 10, 9, 8, 7, 6, 5, 4, 3, 2, or 1, or 0 of the amino acids can be replaced by any amino acid.

In another embodiment thereof, the present invention relates to a polypeptide comprising or consisting of a consensus sequence shown in the respective line in Table IV, application no. 15, column 8 or comprising at least one polypeptide motif shown in the respective line in Table IV, application no. 15, column 8, whereby not more than 15%, 10%, 5%, 4%, 3%, 2% or1% or 0% of the amino acids defined as distinct specific amino acids are/is replaced by another amino acid.

In another embodiment thereof, the present invention relates to a polypeptide comprising or consisting of a consensus sequence shown in the respective line in Table IV, application no. 15, column 8 or comprising at least one polypeptide motif shown in the respective line in Table IV, application no. 15, column 8, whereby not more than 20, 15, 10, 9, 8, 7, 6, 5, 4, 3, 2, or 1, or 0 amino acids are inserted into the consensus sequence or polypeptide motif.

In an embodiment of the present invention, the polypeptide of the present invention comprises or consists of a consensus sequence or at least one polypeptide motif shown in the respective line in Table IV, application no. 15, column 8, whereby said polypeptide distinguishes over the sequence depicted in the respective line in Table II, application no. 15, columns 5 or 8 by one or more amino acids. In one embodiment, said polypeptide of the invention distinguishes over the sequence shown in the respective line in Table II, application no. 15, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 15, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in the respective line in Table II, application no. 15, columns 5 and 8.

In an embodiment, the invention relates to a polypeptide conferring an increase in the respective fine chemical citramalate, citrate, fumarate, glycerate, malate, oxalate, pyruvate, succinate, or vitamin c and/or threonic acid in a non-human organism, especially a microorganism or a plant, or a part thereof, being encoded by the nucleic acid molecule of the invention or used in the process of the invention and having a sequence which distinguishes over the sequence as shown in the respective line in Table II, application no. 15, columns 5 or 8 by one or more amino acids (but not exceeding 5%, preferably 4%, 3%, 2%, 1%, 0.5%, 0.2% or 0.1%). In an embodiment, said polypeptide of the invention does not comprise or consist of the sequence shown in the respective line in Table II, application no. 15, columns 5 or 8. In an embodiment, said polypeptide of the present invention is less than 100%, 99.999%, 99.99%, 99.9% or 99% identical. In one embodiment, said polypeptide which differs at least in one or more amino acids (but not exceeding 5%, preferably 4%, 3%, 2%, 1%, 0.5%, 0.2% or 0.1%) from the polypeptide shown in the respective line in Table II, application no. 15, columns 5 and 8 does not comprise a protein of the sequence shown in the respective line in Table II A and/or II B, application no. 15, columns 5 or 8.

In an embodiment, the present invention relates to a polypeptide having the activity of the protein as shown in the respective line in Table II, application no. 15, column 3, which distinguishes over the sequence depicted in the respective line in Table II, application no. 15, columns 5 or 8 by one or more amino acids, preferably by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 15, columns 5 and 8 by not more than 80%, 70%, 60%, 50%, 40%, 30%, 20%, 10%, 5%, 4%, 3%, 2%, 1% or 0.5%.

In another embodiment the polypeptide of the invention takes the form of a preprotein consisting of a plastidial or mitochondrial transit peptide joint to a polypeptide having the activity of the protein as shown in Table II, column 3, from which the transit peptide is preferably cleaved off upon transport of the preprotein into the organelle for example into the plastid or mitochondrion.

In another embodiment the polypeptide of the invention takes not the form of a preprotein.

for the disclosure of this paragraph see [0390.1.7.7] above

for the disclosure of this paragraph see [0391.1.7.7] above

A polypeptide of the invention can participate in the process of the present invention. The polypeptide or a portion thereof comprises preferably an amino acid sequence, which is sufficiently homologous to an amino acid sequence shown in the respective line in Table II A and/or II B, application no. 15, columns 5 or 8.

Further, the polypeptide can have an amino acid sequence which is encoded by a nucleotide sequence which hybridizes, preferably hybridizes under stringent conditions as described above, to a nucleotide sequence of the nucleic acid molecule of the present invention.

Accordingly, the polypeptide has an amino acid sequence which is encoded by a nucleotide sequence that is at least about 35%, 40%, 45%, 50%, 55%, 60%, 65%, 70%, 75%, 80%, 85%, 90%, 91%, 92%, 93%, 94%, 95%, 96%, 97%, 98%, 99%, 99.5% or more homologous to one of the amino acid sequences as shown in the respective line in Table II A and/or II B, application no. 15, columns 5 or 8, or fragments thereof. The preferred polypeptide of the present invention preferably possesses at least one of the activities according to the invention and described herein. A preferred polypeptide of the present invention includes an amino acid sequence encoded by a nucleotide sequence which hybridizes, preferably hybridizes under stringent conditions, to a nucleotide sequence shown in the respective line in Table I A and/or I B, application no. 15, columns 5 or 8, preferably the coding region thereof, or fragments thereto or which is homologous thereto, as defined above

The polypeptide of the present invention can vary from the sequences shown in the respective line in Table II A and/or II B, application no. 15, columns 5 or 8 in amino acid sequence due to natural variation or mutagenesis, as described in detail herein. Accordingly, the polypeptide comprise an amino acid sequence which is at least about 35%, 40%, 45%, 50%, 55%, 60%, 65%, 70%, 75%, 80%, 85% or 90, 91%, 92%, 93%, 94%, 95%, 96%, 97%, 98%, 99%, 99.5% or more homologous to an entire amino acid sequence shown in the respective line in Table II A and/or II B, application no. 15, columns 5 or 8.

Biologically active portions of a polypeptide of the present invention include peptides comprising amino acid sequences derived from the amino acid sequence of the polypeptide of the present invention or used in the process of the present invention, e.g., the amino acid sequence shown in the respective line in Table II, application no. 15, columns 5 or 8 or the amino acid sequence of a protein homologous thereto, which include fewer amino acids than a full length polypeptide of the present invention or used in the process of the present invention or the full length protein which is homologous to a polypeptide of the present invention or used in the process of the present invention depicted herein, and exhibit at least one activity of a polypeptide of the present invention or used in the process of the present invention.

for the disclosure of this paragraph see [0396.1.7.7] above

Manipulation of the nucleic acid molecule of the invention may result in the production of a protein having differences from the protein as shown in the respective line in Table II, application no. 15, column 5 or 8. Differences shall mean at least one amino acid different from the sequences as shown in the respective line in Table II, application no. 15, column 5 or 8, preferably at least 2, 3, 4, 5, 6, 7, 8, 9 or 10 amino acids (especially but not exceeding 0.1%, preferably 0.2%, 0.5%, 1%, 2%, 3% 4% or 5%), more preferably at least 15, 20, 25, 30, 35, 40, 45 or 50 amino acids (especially but not exceeding 0.5%, preferably 1%, 2%, 3% 4%, 5%, 10% or 25%) different from the sequences as shown in the respective line in Table II, application no. 15, column 5 or 8. These proteins may be improved in efficiency or activity, or may be more stable and therefore present in greater numbers in the cell as compared to the wild-type cell.

Any mutagenesis strategies for the polypeptide of the present invention or the polypeptide used in the process of the present invention to result in increasing said activity are not meant to be limiting; variations on these strategies will be readily apparent to one skilled in the art. Using such strategies, and incorporating the mechanisms disclosed herein, the nucleic acid molecule and polypeptide of the invention may be utilized to generate algae, ciliates, fungi, other microorganisms like C. glutamicum or plants or parts thereof, expressing proteins as shown in the respective line in Table II, application no. 15, column 5 or 8, or mutated proteins thereof. The nucleic acid molecules and polypeptide molecules of the invention are expressed such that the yield, production, and/or efficiency of production of a desired compound is improved.

[0399.1.7.15] to [0401.1.7.15] for the disclosure of theses paragraphs see [0399.1.7.7] to [0401.1.7.7] above

In one embodiment, a protein (=polypeptide) as shown in the respective line in Table II, application no. 15, column 5 or 8 refers to a polypeptide having an amino acid sequence corresponding to the polypeptide of the invention, especially to a polypeptide as shown in Table II, application no. 15, columns 5 or 8 or homologs thereof, or used in the process of the invention, whereas a “non-inventive protein or polypeptide” or “other polypeptide” refers to a polypeptide having an amino acid sequence corresponding to a protein which is not substantially homologous to a polypeptide of the invention, preferably which is not substantially homologous to a polypeptide or protein as shown in the respective line in Table II, application no. 15, column 5 or 8 or homologs thereof, e.g., a protein which does not confer the activity described herein and which is derived from the same or a different organism.

[0403.1.7.15] to [0409.1.7.15] for the disclosure of these paragraphs see [0403.1.7.7] to [0409.1.7.7] above

As the nucleic acid molecules of the invention and/or the expression thereof is related to the synthesis of the respective fine chemical citramalate, citrate, fumarate, glycerate, malate, oxalate, pyruvate, succinate, or vitamin c and/or threonic acid its function as a probe extends to the detection of microorganisms, plant tissues, plants, plant variets, plant ecotypes or plant genera with varying, advantageously increased, capability or potential for synthesis of the respective fine chemical citramalate, citrate, fumarate, glycerate, malate, oxalate, pyruvate, succinate, or vitamin c and/or threonic acid. Therefore in one embodiment the present invention relates to a method for analyzing the capability or potential of a plant tissue, a plant, a plant variety or ecotype to produce the respective fine chemical citramalate, citrate, fumarate, glycerate, malate, oxalate, pyruvate, succinate, or vitamin c and/or threonic acid by using the respective antibody of the invention as a probe to detect the amount of the polypeptide encoded by said nucleic acid molecule of the invention in a non-human organism in comparison to another organism.

Therefore in one embodiment the invention relates to a method to probe a non-human organism for its capability for the production of the respective fine chemical comprising the steps of
a) analyzing the sequence and/or the expression of the respective nucleic acid molecule of the invention in least two non-human organisms which differ in the capability to produce the respective fine chemical;
b) associate the different sequence and/or expression level of the nucleic acid molecules of the invention with their different capability to produce the respective fine chemical;
c) analyze the sequence and/or the expression of the nucleic acid molecule of the invention in at least a third non-human organism;
d) predict the capability of the at least third organism to produce the respective fine chemical based by comparing the sequence and/or expression of the nucleic acid of the invention to the sequences and/or expression levels of the nucleic acid molecule of the invention of the at least two organisms analyzed in step a.

[0411.1.7.15] to [0430.1.7.15] for the disclosure of these paragraphs see [0411.1.7.7] to [0430.1.7.7] above

Accordingly the present invention relates to any cell transgenic for any nucleic acid characterized as part of the invention, e.g. conferring the increase of the respective fine chemical citramalate, citrate, fumarate, glycerate, malate, oxalate, pyruvate, succinate, or vitamin c and/or threonic acid in a cell or a non-human organism or a part thereof, e.g. the nucleic acid molecule of the invention, the nucleic acid construct of the invention, the vector of the invention, the expression cassette according to the invention, or a nucleic acid molecule encoding the polypeptide of the invention, e.g. encoding a polypeptide having an activity as the protein as shown in the respective line in Table II, application no. 15, column 3. Due to the abovementioned activity the respective fine chemical citramalate, citrate, fumarate, glycerate, malate, oxalate, pyruvate, succinate, or vitamin c and/or threonic acid content in a cell or a non-human organism is increased. For example, due to modulation or manipulation, the cellular activity is increased, in a preferred embodiment in organelles such as plastids or mitochondria, e.g. due to an increased expression or specific activity or specific targeting of the subject matters of the invention in a cell or a non-human organism or a part thereof especially in organelles such as plastids or mitochondria, or in another embodiment in the cytosol. Transgenic for a polypeptide having a protein or a protein activity means herein that due to modulation or manipulation of the genome, the activity of protein as shown in the respective line in Table II, application no. 15, column 3 or a protein as shown in the respective line in Table II, application no. 15, column alike activity is increased in the cell or non-human organism or part thereof, especially in organism elles such as plastids or mitochondria, or especially in the cytosol. Examples are described above in context with the process of the invention.

for the disclosure of this paragraph see [0432.1.7.7] above

A naturally occurring expression cassette—for example the naturally occurring combination of the promoter of the gene encoding a protein as shown in the respective line in Table II, application no. 15, column 5 or 8, preferably the coding region thereof, or fragments or homologs thereof with the corresponding encoding gene—becomes a transgenic expression cassette when it is modified by non-natural, synthetic “artificial” methods such as, for example, mutagenesis. Such methods have been described (U.S. Pat. No. 5,565,350; WO 00/15815; also see above).

for the disclosure of this paragraph see [0434.1.7.7] above

for the disclosure of this paragraph see [0435.1.7.7] above

Transgenic plants comprising the respective fine chemical(s) synthesized in the process according to the invention can be marketed directly without isolation of the fine chemical synthesized. In the process according to the invention, plants are understood as meaning all plant parts, plant organs such as leaf, stalk, root, tubers or seeds or propagation material or harvested material or the intact plant. In this context, the seed encompasses all parts of the seed such as the seed coats, epidermal cells, seed cells, endosperm or embryonic tissue. The fine chemical produced in the process according to the invention may, however, also be isolated from the plant. In case of citramalate, citrate, fumarate, glycerate, malate, oxalate, pyruvate, succinate, or vitamin c and/or threonic acid this can be in free form or bound to proteins. Fine chemical(s) produced by this process can be harvested by harvesting the non-human organisms either from the culture in which they grow or from the field. For example, this can be done via squeezing, grinding and/or extraction, salt precipitation and/or ion-exchange chromatography of the plant parts, preferably the plant seeds, plant fruits, plant tubers and the like.

[0437.1.7.15] to [0447.1.7.15] for the disclosure of these paragraphs see [0437.1.7.7] to [0447.1.7.7] above

The identity and purity of the isolated compound(s) can be determined by prior art techniques. These include high performance liquid chromatography (HPLC), spectroscopic methods, mass spectrometry (MS), staining methods, thin-layer chromatography, NIRS, enzyme assay or microbiological assays. These analytical methods are summarized for example in: Patek et al., Appl. Environ. Microbiol. 60, 133 (1994), Malakhova et al., Biotekhnologiya 11, 27 (1996); Schmidt et al., Bioprocess Engineer. 19, 67 (1998), Ullmann's Encyclopedia of Industrial Chemistry (1996) Vol. A27, VCH, Weinheim, pp. 89-90, pp. 521-540, pp. 540-547, pp. 559566, 575-581 and pp. 581-587, Michal G., “Biochemical Pathways: An Atlas of Biochemistry and Molecular Biology”, John Wiley and Sons (1999); Fallon A. et al. “Applications of HPLC in Biochemistry” in “Laboratory Techniques in Biochemistry and Molecular Biology”, Vol. 17 (1987). The fine chemical of the invention can for example be analyzed advantageously via HPLC or GC separation methods and detected by MS or MSMS methods. The unambiguous detection for the presence of the fine chemical containing products can be obtained by analyzing recombinant organisms using analytical standard methods: GC, GC-MS or TLC, as described on several occasions by Christie and the references therein (1997, in: Advances on Lipid Methodology, Fourth Edition: Christie, Oily Press, Dundee, 119-169; 1998, Gaschromatographie-Massenspektrometrie-Verfahren [Gas chromatography/mass spectrometric methods], Lipide 33:343-353). The material to be analyzed can be disrupted by sonication, grinding in a glass mill, liquid nitrogen and grinding, cooking, or via other applicable methods; see also Biotechnology of Vitamins, Pigments and Growth Factors, Edited by Erik J. Vandamme, London, 1989, p.96 to 103.

The fine chemical obtained in the process are suitable as starting material for the synthesis of further products of value. For example, in case of vitamin c or ascorbate and derivates and/or threonic acid they can be used in combination with each other or alone for the production of pharmaceuticals, foodstuffs, animal feeds or cosmetics.

The fine chemical vitamin c or ascorbate and derivates and/or threonic acid can then be processed further directly into foodstuffs or animal feeds or for other applications, for example according to the disclosures made in U.S. Pat. No. 6,399,059: Thermally stable enzyme composition and method of preparing the same, U.S. Pat. No. 6,361,800: Multi-vitamin and mineral supplement , U.S. Pat. No. 6,348,200: Cosmetic composition, U.S. Pat. No. 6,338,854: Photoaging skin-care preparation and method of treating wrinkled skin, U.S. Pat. No. 6,323,188: Treatment and prevention of cardiovascular diseases, heart attack, and stroke, primary and subsequent, with help of aspirin and certain vitamins, U.S. Pat. No. 6,299,896: Multi-vitamin and mineral supplement, U.S. Pat. No. 6,344,573: Process for extraction and concentration of liposoluble vitamins and provitamins, growth factors and animal and vegetable hormones from residues and by-products of industrialized animal and vegetable products, U.S. Pat. No. 6,242,227: Method of vitamin production, which are expressly incorporated herein by reference. The fermentation broth, fermentation products, plants or plant products can be purified as described in above mentioned applications or by other methods known to the person skilled in the art and described herein below.

Accordingly, the present invention relates a method for the production of a pharmaceuticals, food stuff, animal feeds, nutrients or cosmetics comprising the steps of the process according to the invention, including the isolation of the fine chemical produced or a composition produced comprising the fine chemical, and, if desired, formulating the product with a pharmaceutical acceptable carrier or formulating the product in a form acceptable for an application in agriculture or formulating the product in a form acceptable for an application as food stuff, animal feed, nutrient or cosmetic. A further embodiment according to the invention is the use of the fine chemical produced in the process or of the transgenic non-human organisms in animal feeds, foodstuffs, medicines, food supplements, cosmetics or pharmaceuticals.

A further embodiment according to the invention is the use of L-threonic acid for increasing the action or “potency” of vitamin C. Simultaneous feeding of threonic acid and ascorbate can enhance ascorbate uptake.

L-threonic acid calcium is a major calcium treatment project.Threonic acid is a natural constituent in leaves of certain plants. It is derived from ascorbate either by enzymatic or natural oxidation.The metabolism of ascorbic acid involves its oxidation to dehydroascorbic acid, followed by the formation of the aldonic acids L-lyxonic acid, L-xylonic acid, and L-threonic acid.

A further embodiment according to the invention is the use of the fumarate as substrate in electrochemical bioreactor systems.

A further embodiment according to the invention is the use of glyceric acid in glycerate-based surfactants, which are a new class of swelling amphiphiles which swell to a finite degree with water. It is further used as an unusual acyl substituent in gellan gum, or in glycerate phosphates as activated precursor for biosynthetic processes.

A further embodiment according to the invention is the use of pyruvate as starting material for the production of lactate which then can be used to produce acrylates such as sealants, coatings, textiles, and biodegradable plastics.

A further embodiment according to the invention is the use of succinate to produce succinate semialdehyde, 1,4-butanediol (BDO) and related products, tetrahydrofuran and y-butyrolactone, N-methyl pyrrolidinone (NMP) and 2-pyrrolidinone, adipic acid, linear aliphatic succinate esters and related compounds that can serve as precursors for industrial production of polymers. Succinate salts are further used according to the invention as de-icers and herbicide additives.

A further embodiment according to the invention is the use of citrate (E330) in foodstuff for conservation and to create a soure taste. Further embodiment is the use of citrate pharmaceutically used as a dispersing agent for creation of stable suspensions. A further embodiment according to the invention is the use of citrate as a component in buffer solutions, including the commonly used SSC 20×hybridization buffer. A further embodiment according to the invention is the use of citrate trinatrium- and trilithiumsalts to modify the hardening speed of concrete and other cementitious materials.

A further embodiment according to the invention is the use of oxalate to reduce dentin permeability under adhesive restorations.

[0450.1.7.15] to [0452.1.7.15] for the disclosure of these paragraphs see [0450.1.7.7] to [0452.1.7.7] above

Further in another embodiment, the present invention relates to the use of the nucleic acid molecule of the invention or used in the method of the invention alone or in combination with other genes of the respective fine chemical synthesis for example of the biosynthesis of a substrate or product of the citric acid cycle, a derivate of one of the substrates or products (for example pyruvate and malate as derivates of citramalate, acetyl-CoA as derivate of pyruvate or citrate, pyruvate as derivate of glycerate) or vitamin c, ascorbic acid or ascorbate and derivates like oxalate and/or threonic acid, the polypeptide of the invention or used in the method of the invention, the nucleic acid construct of the invention, the expression cassette of the invention, the vector of the invention, the plant or plant tissue or the host cell of the invention, for the production of plant resistant to a herbicide inhibiting eventually the production of the fine chemical.

for the disclosure of this paragraph see [0454.1.7.7] above

In one embodiment, the present invention relates to a method for the identification of a gene product conferring the production of or an increase in the fine chemical production in a non-human cell, comprising the following steps:
(a) contacting, e.g. hybridizing, a, some or all nucleic acid molecules of a sample, e.g. cells, tissues, plants or microorganisms or a nucleic acid library, which can contain a candidate gene encoding a gene product conferring the production of or an increase in the fine chemical after expression, with the nucleic acid molecule of the present invention as shown in the respective line in column 5 or 8 of Table I A or B of application no. 15, preferably the coding region thereof, or a fragment or homolog thereof;
(b) identifying the nucleic acid molecules, which hybridize under relaxed stringent conditions with the nucleic acid molecule of the present invention in particular to the nucleic acid molecule sequence shown in the respective line in Table I, application no. 15, columns 5 or 8, preferably the coding region thereof, of a fragment or a homolog thereof, preferably in Table I B, application no. 15, columns 5 or 8, preferably the coding region thereof, of a fragment or a homolog thereof, and, optionally, isolating the full length cDNA clone or complete genomic clone;
(c) introducing the candidate nucleic acid molecules or a fragment thereof in host cells, preferably in a plant cell or a microorganism, appropriate for producing the fine chemical;
(d) expressing the identified nucleic acid molecules in the host cells for which the production of or the increased production of the fine chemical is desired;
(e) assaying the fine chemical level in the non-human host cells; and
(f) identifying the nucleic acid molecule and/or its gene product which expression confers the production of or an increase in the fine chemical level in the non-human host cell after expression compared to the wild type.

for the disclosure of this paragraph see [0456.1.7.7] above

In another embodiment, the present invention relates to a method for the identification of a gene product conferring the production of or an increase in the fine chemical production in a cell, comprising the following steps:
(a) identifying a nucleic acid molecule of a non-human organism, which is at least 20%, 25%, 30%, 35%, 40%, 50%, 60%, 70%, 80%, 90%, 95%, 96%, 97%, 98%, 99%, 99.5% or more homolog to the nucleic acid molecule encoding a polypeptide comprising the polypeptide molecule as shown in the respective line in column 5 or 8 of Table II, application no. 15, or comprising a consensus sequence or a polypeptide motif as shown in the respective line in column 8 of Table IV, application no. 15, or being encoded by a nucleic acid molecule comprising a polynucleotide as shown in the respective line in column 5 or 8 of Table I, application no. 15, preferably the coding region thereof, or a homologue thereof as described herein, for example via homology search in a data bank;
(b) introducing the candidate nucleic acid molecule in non-human host cells, preferably in a plant cell or a microorganism, appropriate for producing the fine chemical;
(c) expressing the identified nucleic acid molecule in the non-human host cells;
(d) assaying the fine chemical level in the non-human host cells; and
(e) identifying the nucleic acid molecule and its gene product which expression confers the production of or an increase in the fine chemical level in the non-human host cell after expression compared to the wild type.

for the disclosure of this paragraph see [0458.1.7.7] above

Further, the nucleic acid molecules disclosed herein, in particular the nucleic acid molecules shown in the respective line in column 5 or 8 of Table I A or B of application no. 15, may be sufficiently homologous to the sequences of related species such that these nucleic acid molecules may serve as markers for the construction of a genomic map in related organism or for association mapping. Furthermore natural variation in the genomic regions corresponding to nucleic acids disclosed herein, in particular the nucleic acid molecule shown in the respective line in column 5 or 8 of Table I A or B, of application no. 15 or homologous thereof, may lead to variation in the activity of the proteins disclosed herein, in particular the proteins comprising polypeptides as shown in the respective lines in column 5 or 8 of Table II A or B, of application no. 15 or their homologous, or comprising the consensus sequence or the polypeptide motif as shown in the respective line in column 8 of Table IV, and in consequence in a natural variation of an increased fine chemical production.

In consequence natural variation eventually also exists in form of more active allelic variants leading already to a relative increase in the fine chemical. Different variants of the nucleic acid molecules disclosed herein, in particular the nucleic acid molecules comprising the nucleic acid molecules as shown in the respective line in column 5 or 8 of Table I A or B of application no. 15, preferably the coding region thereof, which corresponds to different levels of increase in fine chemical, e.g. different levels of increase in fine chemical, can be indentified and used for marker assisted breeding for an enhanced production of the fine chemical.

Natural variation may also exist in the regulatory regions, e.g. the promoters of the natural genes comprising the nucleic acid molecules as shown in the respective line column 5 or 8 of Table I A or B of application no. 15, preferably the coding region thereof, which can similarly correspond to different levels of increase in the fine chemical, e.g. different levels of increase in the fine chemical can be identified and used for marker assisted breeding for an enhanced production of the fine chemical.

Accordingly, the present invention relates to a method for breeding plants for the production of the fine chemical, comprising
(a) selecting a first plant variety with an increased production of the fine chemical, e.g. based on an increased expression of a nucleic acid molecule of the invention as disclosed herein, in particular of a nucleic acid molecule comprising a nucleic acid molecule as shown in the respective line in column 5 or 8 of Table I A or B of application no. 15, preferably the coding region thereof, or fragments or homologs thereof, or a polypeptide comprising a polypeptide as shown in the respective line in column 5 or 8 of Table II A or B of application no. 15, or fragments or homologs thereof, or comprising a consensus sequence or a polypeptide motif as shown in the respective line in column 8 of Table IV, or a homolog thereof, as described herein;
(b) associating the level of increased production of the fine chemical, with the expression level or the genomic structure of a gene encoding said polypeptide or said nucleic acid molecule;
(c) crossing the first plant variety with a second plant variety, which significantly differs in its level of fine chemical; and
(d) identifying, which of the offspring varieties has got increased production of the fine chemical by the expression level of said polypeptide or nucleic acid molecule or the genomic structure of the gene(s) encoding said polypeptide or nucleic acid molecule of the invention.

[0462.1.7.15] to [0479.1.7.15] for the disclosure of these paragraphs see [0462.1.7.7] to [0479.1.7.7] above

In a further embodiment the nucleic acid molecule of the invention, the polypeptide of the invention, the nucleic acid construct of the invention, the expression cassette of the invention, the non-human organisms, the non-human host cell, the microorganisms, the plant, plant cell, or the part thereof, of the invention, the vector of the invention, the agonist identified with the method of the invention, the nucleic acid molecule identified with the method of the present invention, can be used for the production of the fine chemical or of the fine chemical and one or more substrate or product of the citric acid cycle, a derivate of one of the substrates or products or vitamin c or derivates of ascorbic acid and/or threonic acid.

Accordingly, the nucleic acid molecule of the invention, or the nucleic acid molecule identified with the method of the present invention or the complement sequences thereof, the polypeptide of the invention, the nucleic acid construct of the invention, the expression cassette of the invention, the non-human organism, the non-human host cell, the microorganism, the plant, plant cell, or the part thereof of the invention, the vector of the invention, the agonist identified with the method of the invention, the antibody of the present invention, can be used for the production of or the increase in the fine chemical in a non-human organism or part thereof, e.g. in a cell.

for the disclosure of this paragraph see [0481.1.7.7] above

for the disclosure of this paragraph see [0482.1.7.7] above

A special embodiment of this invention is related to genes which increase or generate the production of the fine chemical citramalate, citrate, fumarate, malate and/or succinate in plant cells, plants or part thereof. Phenotypes thereto are associated with yield of plants (=yield related phenotypes). In accordance with the invention, therefore, the respective genes identified in Table I, wherein in column 7 citramalate, citrate, fumarate, malate and/or succinate are mentioned, especially the coding region thereof, or homologs or fragments thereof,may be employed to enhance any yield-related phenotype. Increased yield may be determined in field trials of transgenic plants and suitable control plants. Alternatively, a transgene's ability to increase yield may be determined in a model plant. An increased yield phenotype may be determined in the field test or in a model plant by measuring any one or any combination of the following phenotypes, in comparison to a control plant: yield of dry harvestable parts of the plant, yield of dry aerial harvestable parts of the plant, yield of underground dry harvestable parts of the plant, yield of fresh weight harvestable parts of the plant, yield of aerial fresh weight harvestable parts of the plant yield of underground fresh weight harvestable parts of the plant, yield of the plant's fruit (both fresh and dried), grain dry weight, yield of seeds (both fresh and dry), and the like.

The most basic yield-related phenotype is increased yield associated with the presence of the gene or a homolog or a fragment thereof as a transgene in the plant, i.e., the intrinsic yield of the plant. Intrinsic yield capacity of a plant can be, for example, manifested in a field test or in a model system by demonstrating an improvement of seed yield (e.g. in terms of increased seed/grain size, increased ear number, increased seed number per ear, improvement of seed filling, improvement of seed composition, embryo and/or endosperm improvements, and the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under non-stressed conditions), improvement in plant architecture.

Increased yield-related phenotypes may also be measured to determine tolerance to abiotic environmental stress. Abiotic stresses include drought, low temperature, salinity, osmotic stress, shade, high plant density, mechanical stresses, and oxidative stress, and yield-related phenotypes are encompassed by tolerance to such abiotic stresses. Additional phenotypes that can be monitored to determine enhanced tolerance to abiotic environmental stress include, without limitation, wilting; leaf browning; loss of turgor, which results in drooping of leaves or needles stems, and flowers; drooping and/or shedding of leaves or needles; the leaves are green but leaf angled slightly toward the ground compared with controls; leaf blades begun to fold (curl) inward; premature senescence of leaves or needles; loss of chlorophyll in leaves or needles and/or yellowing. Any of the yield-related phenotypes described above may be monitored in field tests or in model plants to demonstrate that a transgenic plant has increased tolerance to abiotic environmental stress. In accordance with the invention, the respective genes identified in Table 1, in case in column 7 citramalate, citrate, fumarate, malate and/or succinate is indicated, especially the coding region thereof, or homologs or fragments thereof, may be employed to enhance tolerance to abiotic environmental stress in a plant means that the plant, when confronted with abiotic environmental stress.

A polypeptide conferring an yield-increasing activity can be encoded by a respective nucleic acid sequence as shown in Table I, column 5 or 8, and/or comprises or consists of a respective polypeptide as depicted in Table II, column 5 and 8, and/or can be amplified with the respective primer set shown in Table III, column 8, in case in column 7 citramalate, citrate, fumarate, malate and/or succinate is indicated.

“Improved adaptation” to environmental stress like e.g. freezing and/or chilling temperatures refers to an improved plant performance under environmental stress conditions.

A modification, i.e. an increase, can be caused by endogenous or exogenous factors. For example, an increase in activity in an organism or a part thereof can be caused by adding a gene product or a precursor or an activator or an agonist to the media or nutrition or can be caused by introducing said subjects into an organism, transient or stable. Furthermore such an increase can be reached by the introduction of the respective inventive nucleic acid sequence or the encoded protein in the correct cell compartment for example into the nucleus or cytoplasmic respectively or into plastids either by transformation and/or targeting.

The term “yield” as used herein generally refers to a measurable produce from a plant, particularly a crop. Yield and yield increase (in comparison to a non-transformed starting or wild-type plant) can be measured in a number of ways, and it is understood that a skilled person will be able to apply the correct meaning in view of the particular embodiments, the particular crop concerned and the specific purpose or application concerned. The terms “improved yield” or “increased yield” can be used interchangeable.

As used herein, the term “improved yield” or the term “increased yield” means any improvement in the yield of any measured plant product, such as grain, fruit or fiber. In accordance with the invention, changes in different phenotypic traits may improve yield. For example, and without limitation, parameters such as floral organ development, root initiation, root biomass, seed number, seed weight, harvest index, tolerance to abiotic environmental stress, leaf formation, phototropism, apical dominance, and fruit development, are suitable measurements of improved yield. Any increase in yield is an improved yield in accordance with the invention. For example, the improvement in yield can comprise a 0.1%, 0.5%, 1%, 3%, 5%, 10%, 15%, 20%, 30%, 40%, 50%, 60%, 70%, 80%, 90% or greater increase in any measured parameter. For example, an increase in the bushel/acre yield of soybeans or corn derived from a crop comprising plants which are transgenic for the nucleotides and polypeptides of Table I, in case in column 7 citramalate, citrate, fumarate, malate and/or succinate is mentioned, as compared with the bushel/acre yield from untreated soybeans or corn cultivated under the same conditions, is an improved yield in accordance with the invention. The increased or improved yield can be achieved in the absence or presence of stress conditions.

For example, enhanced or increased “yield” refers to one or more yield parameters selected from the group consisting of biomass yield, dry biomass yield, aerial dry biomass yield, underground dry biomass yield, fresh-weight biomass yield, aerial fresh-weight biomass yield, underground fresh-weight biomass yield; enhanced yield of harvestable parts, either dry or freshweight or both, either aerial or underground or both; enhanced yield of crop fruit, either dry or fresh-weight or both, either aerial or underground or both; and preferably enhanced yield of seeds, either dry or fresh-weight or both, either aerial or underground or both.

Crop yield is defined herein as the number of bushels of relevant agricultural product (such as grain, forage, or seed) harvested per acre. Crop yield is impacted by abiotic stresses, such as drought, heat, salinity, and cold stress, and by the size (biomass) of the plant. Traditional plant breeding strategies are relatively slow and have in general not been successful in conferring increased tolerance to abiotic stresses. Grain yield improvements by conventional breeding have nearly reached a plateau in maize.

Accordingly, the yield of a plant can depend on the specific plant/crop of interest as well as its intended application (such as food production, feed production, processed food production, biofuel, biogas or alcohol production, or the like) of interest in each particular case. Thus, in one embodiment, yield is calculated as harvest index (expressed as a ratio of the weight of the respective harvestable parts divided by the total biomass), harvestable parts weight per area (acre, square meter, or the like); and the like. The harvest index, i.e., the ratio of yield biomass to the total cumulative biomass at harvest, in maize has remained essentially unchanged during selective breeding for grain yield over the last hundred years. Accordingly, recent yield improvements that have occurred in maize are the result of the increased total biomass production per unit land area. This increased total biomass has been achieved by increasing planting density, which has led to adaptive phenotypic alterations, such as a reduction in leaf angle, which may reduce shading of lower leaves, and tassel size, which may increase harvest index. Harvest index is relatively stable under many environmental conditions, and so a robust correlation between plant size and grain yield is possible. Plant size and grain yield are intrinsically linked, because the majority of grain biomass is dependent on current or stored photosynthetic productivity by the leaves and stem of the plant. As with abiotic stress tolerance, measurements of plant size in early development, under standardized conditions in a growth chamber or greenhouse, are standard practices to measure potential yield advantages conferred by the presence of a transgene.

Acccordingly, the yield of a plant can be increased by improving one or more of the yield-related phenotypes.

Such yield-related phenotypes or traits of a plant the improvement of which results in increased yield comprise, without limitation, the increase of the intrinsic yield capacity of a plant, improved nutrient use efficiency, and/or increased stress tolerance.

For example, yield refers to biomass yield, e.g. to dry weight biomass yield and/or fresh-weight biomass yield. Biomass yield refers to the aerial or underground parts of a plant, depending on the specific circumstances (test conditions, specific crop of interest, application of interest, and the like). In one embodiment, biomass yield refers to the aerial and underground parts. Biomass yield may be calculated as fresh-weight, dry weight or a moisture adjusted basis. Biomass yield may be calculated on a per plant basis or in relation to a specific area (e.g. biomass yield per acre/square meter/ or the like).

In other embodiment, “yield” refers to seed yield which can be measured by one or more of the following parameters: number of seeds or number of filled seeds (per plant or per area (acre/square meter/ or the like)); seed filling rate (ratio between number of filled seeds and total number of seeds); number of flowers per plant; seed biomass or total seeds weight (per plant or per area (acre/square meter/ or the like); thousand kernel weight (TKW; extrapolated from the number of filled seeds counted and their total weight; an increase in TKW may be caused by an increased seed size, an increased seed weight, an increased embryo size, and/or an increased endosperm). Other parameters allowing to measure seed yield are also known in the art. Seed yield may be determined on a dry weight or on a fresh weight basis, or typically on a moisture adjusted basis, e.g. at 15.5 percent moisture.

For example, the term “increased yield” means that the plant, exhibits an increased growth rate, under conditions of abiotic environmental stress, compared to the corresponding wild-type plant.

An increased growth rate may be reflected inter alia by or confers an increased biomass production of the whole plant, or an increased biomass production of the aerial parts of a plant, or by an increased biomass production of the underground parts of a plant, or by an increased biomass production of parts of a plant, like stems, leaves, blossoms, fruits, and/or seeds. Increased yield includes higher fruit yields, higher seed yields, higher fresh matter production, and/or higher dry matter production.

The term “increased yield” means that the plant, exhibits a prolonged growth under conditions of abiotic environmental stress, as compared to the corresponding, e.g. non-transformed, wild type organism. A prolonged growth comprises survival and/or continued growth of the plant, at the moment when the non-transformed wild type organism shows visual symptoms of deficiency and/or death.

When the plant of the invention is a corn plant increased yield for corn plants means, for example, increased seed yield, in particular for corn varieties used for feed or food. Increased seed yield of corn refers to an increased kernel size or weight, an increased kernel per ear, or increased ears per plant. Alternatively or in addition the cob yield may be increased, or the length or size of the cob is increased, or the kernel per cob ratio is improved.

When the plant of the invention is a soy plant, increased yield for soy plants means increased seed yield, in particular for soy varieties used for feed or food. Increased seed yield of soy refers for example to an increased kernel size or weight, an increased kernel per pod, or increased pods per plant.

When the plant of the invention is an oil seed rape (OSR) plant, increased yield for OSR plants means increased seed yield, in particular for OSR varieties used for feed or food. Increased seed yield of OSR refers to an increased seed size or weight, an increased seed number per silique, or increased siliques per plant.

When the plant of the invention is a cotton plant,increased yield for cotton plants means increased lint yield. Increased lint yield of cotton refers in one embodiment to an increased length of lint.

When the plant of the invention is a wheat plant increased yield for wheat plants means, for example, increased seed yield, in particular for wheat varieties used for feed or food. Increased seed yield of wheat refers to an increased kernel size or weight, an increased kernel per ear, or increased ears per plant. Increased seed yield of wheat refers also to an increased grain size or weight, an increased number of grains per spike, or increased number of spikes per plant. Alternatively or in addition increased seed yield of wheat refers also to an increased grain yield, increased length of spikes, increased number of spikelets per spike , increased 1000 grain weight or increased flag leaf area (flag leaf is the leaf directely below the spike). Said increased yield can typically be achieved by enhancing or improving, one or more yield-related traits of the plant. Such yield-related traits of a plant comprise, without limitation, the increase of the intrinsic yield capacity of a plant, improved nutrient use efficiency, and/or increased stress tolerance, in particular increased abiotic stress tolerance.

Intrinsic yield capacity of a plant can be, for example, manifested by improving the specific (intrinsic) seed yield (e.g. in terms of increased seed/ grain size, increased ear number, increased seed number per ear, improvement of seed filling, improvement of seed composition, embryo and/or endosperm improvements, or the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under stressed or non-stressed conditions), improvement in plant architecture, cell cycle modifications, photosynthesis modifications, various signaling pathway modifications, modification of transcriptional regulation, modification of translational regulation, modification of enzyme activities, and the like); and/or the like. The improvement or increase of stress tolerance of a plant can for example be manifested by improving or increasing a plant's tolerance against stress, particularly abiotic stress. In the present application, abiotic stress refers generally to abiotic environmental conditions a plant is typically confronted with, including, but not limited to, drought (tolerance to drought may be achieved as a result of improved water use efficiency), heat, low temperatures and cold conditions (such as freezing and chilling conditions), salinity, osmotic stress , shade, high plant density, mechanical stress, oxidative stress, and the like.

The increased plant yield can also be mediated by increasing the “nutrient use efficiency of a plant”, e.g. by improving the use efficiency of nutrients including, but not limited to, phosphorus, potassium, and nitrogen. Further, higher yields may be obtained with current or standard levels of nitrogen use.

Generally, the term “increased tolerance to stress” can be defined as survival of plants, and/or higher yield production, under stress conditions as compared to a non-transformed wild type or starting plant: For example, the plant of the invention or produced according to the method of the invention is better adapted to the stress conditions. “Improved adaptation” to environmental stress like e.g. drought, heat, nutrient depletion, freezing and/or chilling temperatures refers herein to an improved plant performance resulting in an increased yield, particularly with regard to one or more of the yield related traits as defined in more detail above.

During its life-cycle, a plant is generally confronted with a diversity of environmental conditions. Any such conditions, which may, under certain circumstances, have an impact on plant yield, are herein referred to as “stress” condition. Environmental stresses may generally be divided into biotic and abiotic (environmental) stresses. Unfavorable nutrient conditions are sometimes also referred to as “environmental stress”. The present invention does also contemplate solutions for this kind of environmental stress, e.g. referring to increased nutrient use efficiency.

For the purposes of the description of the present invention, the terms “enhanced tolerance to abiotic stress”, “enhanced resistance to abiotic environmental stress”, “enhanced tolerance to environmental stress”, “improved adaptation to environmental stress” and other variations and expressions similar in its meaning are used interchangeably and refer, without limitation, to an improvement in tolerance to one or more abiotic environmental stress(es) as described herein and as compared to a corresponding origin or wild type plant or a part thereof.

The term abiotic stress tolerance(s) refers for example low temperature tolerance, drought tolerance or improved water use efficiency (WUE), heat tolerance, salt stress tolerance and others. Studies of a plant's response to desiccation, osmotic shock, and temperature extremes are also employed to determine the plant's tolerance or resistance to abiotic stresses. Water use efficiency (WUE) is a parameter often correlated with drought tolerance. In selecting traits for improving crops, a decrease in water use, without a change in growth would have particular merit in an irrigated agricultural system where the water input costs were high. An increase in growth without a corresponding jump in water use would have applicability to all agricultural systems. In many agricultural systems where water supply is not limiting, an increase in growth, even if it came at the expense of an increase in water use also increases yield.

Drought stress means any environmental stress which leads to a lack of water in plants or reduction of water supply to plants, including a secondary stress by low temperature and/or salt, and/or a primary stress during drought or heat, e.g. desiccation etc.

### Specific Embodiments

Accordingly, this invention provides respective measures and methods to produce plants with increased yield, e.g. genes conferring an increased yield-related trait, for example enhanced tolerance to abiotic environmental stress, for example an increased drought tolerance and/or low temperature tolerance and/or an increased nutrient use efficiency, intrinsic yield and/or another increased yield-related trait, upon expression or over-expression. Accordingly, the present invention provides such genes in case in column 7 of the respective Table or citramalate, citrate, fumarate, malate and/or succinate is indicated. In particular, such genes are described in column 5 as well as in column 8 of Tables I, especially the coding region thereof, or homologs or fragments thereof, in case citramalate, citrate, fumarate, malate and/or succinate are indicated in column 7 or the respective polypeptides are described in column 5 as well as in column 8 of Table II, or homologs or fragments thereof, in case citramalate, citrate, fumarate, malate and/or succinate are indicated in column 7.

Accordingly, the present invention provides respective transgenic plants showing one or more improved yield-related traits as compared to the corresponding origin or the wild type plant and methods for producing such transgenic plants with increased yield in case in column 7 of the respective Table “citramalate ” or “citrate” or “fumarate”or “malate”or “succinate” is indicated.

In one embodiment, one or more of said yield-increasing activities are increased by increasing the amount and/or the specific activity of one or more proteins listed in Table II, column 5 or 8 in a compartment of a cell indicated in Table II, column 6, in case in column 7 of the respective Table “citramalate ” or “citrate” or “fumarate”or “malate”or “succinate” is indicated.

Accordingly to present invention, the yield of the plant of the invention is increased by improving one or more of the yield-related traits as defined herein. Said increased yield in accordance with the present invention can typically be achieved by enhancing or improving, in comparison to an origin or wild-type plant, one or more yield-related traits of said plant. Such yield-related traits of a plant the improvement of which results in increased yield comprise, without limitation, the increase of the intrinsic yield capacity of a plant, improved nutrient use efficiency, like nitrogen use efficiency, and/or increased stress tolerance.

In one embodiment, the present invention provides respective transgenic plants showing one or more improved yield-related traits, preferably increased drought tolerance as compared to the corresponding origin or the wild type plant and methods for producing such transgenic plants with increased yield in case in column 7 of the respective Table “citramalate” or “citrate” or “fumarate” or “malate” or “succinate” is indicated.

In a further embodiment the present invention is characterized by the features as disclosed in the following items:

Item 1. A process for the production of a fine chemical selected from the group consisting of: citramalate, citrate, fumarate, glycerate, malate, oxalate, pyruvate, succinate and/or vitamin c and/or threonic acid
(a) increasing or generating one or more activities selected from the group consisting of 2-C-methyl-D-erythritol 4-phosphate cytidylyltransferase, 2-isopropylmalate synthase, 2-oxoglutarate dehydrogenase E1 subunit, 30S ribosomal protein S3, 47266012-protein, 49747384_SOYBEAN-protein, 50S ribosomal protein L14, ABC transporter ATP binding component, ABC transporter permease protein, acetyl-CoA acetyltransferase, acetyl-CoA hydrolase, acetyltransferase, acyl-CoA dehydrogenase, acyl-CoA synthase, adenylate kinase, adenylylsulfate kinase, agmatinase, amino-acid acetyltransferase, aminotransferase, ammonium transporter, arginine N-succinyltransferase, aspartase, AO g13930-protein, ATP-binding component of a transport system, auxin response factor, AX653549-protein, AY087308-protein, b0801-protein, b1003-protein, b1285-protein, b1425-protein, b1445-protein, b1522-protein, b1670-protein, b1672-protein, b1742-protein, b1898-protein, b2399-protein, b3777-protein, b3791-protein, b3814-protein, b3817-protein, b3989-protein, b4029-protein, b4050-protein, b4056-protein, beta-hydroxylase, betaine aldehyde dehydrogenase, bifunctional purine biosynthesis protein, branched chain aminotransferase, calcium-dependent protein kinase, cation-transporting ATPase, CBL-interacting protein kinase, CDP-diacylglycerol-glycerol-3-phosphate 3-phosphatidyltransferase, CDP-diacylglycerol-serine O-phosphatidyltransferase, CDP-glucose-4,6-dehydratase, citrate synthase, coproporphyrinogen III oxidase, CTP synthase, cystathionine beta-lyase, cysteine synthase A, cysteine synthase B, dATP pyrophosphohydrolase, dihydrolipoamide dehydrogenase, DnaJ-like chaperone, electron transfer flavoprotein subunit beta, electron transport complex protein, eukaryotic translation initiation factor 5, Fe—S subunit of oxidoreductase, flagellar M-ring protein, fumarate hydratase, gamma-glutamyltranspeptidase, gibberellin 20-oxidase, glucose dehydrogenase, glucose-1-phosphate cytidylyltransferase, glucose-6-phosphate 1-dehydrogenase, glutamate dehydrogenase, glutamate-ammonia-ligase, glutaredoxin, glycerate kinase, glycerol kinase, Glycine cleavage system T aminomethyl-transferase, GM02LC17485-protein, GM02LC5744-protein, heat shock protein, heat shock transcription factor, hexose transporter, hydrolase, isochorismate synthase, isocitrate dehydrogenase, isocitrate lyase, leucyl/phenylalanyl-tRNA-protein transferase, lipopolysaccharide core biosynthesis protein, lipoprotein precursor, lysine decarboxylase, malate dehydrogenase, malonyl CoA-acyl carrier protein transacylase, membrane protein, membrane transport protein, methylglyoxal synthase, methylglyoxalase, methyltransferase, monothiol glutaredoxin, monthiol glutaredoxin, multiple antibiotic resistance protein, murein transglycosylase, NADH-quinone oxidoreductase subunit, O-antigen chain length determinant, oxidoreductase, oxidoreductase subunit, oxireductase, phenylacetic acid degradation protein, phenylalanine ammonia-lyase, phosphatase, phosphate import ATP-binding protein, photosystem I reaction centre subunit, photosystem 11-protein, polygalacturonase, protease, protein phosphatase, purine-nucleoside phosphorylase, pyruvate carboxylase, pyruvate kinase, recombinase A, ribulokinase, RNA polymerase sigma factor, sec-independent protein translocase, Sec-independent protein translocase subunit, slr1170-protein, thioredoxin, thioredoxin family protein, thioredoxin reductase, threonine efflux protein, threonine synthase, transcription factor, transcriptional regulator, transcriptional regulator protein, translation initiation factor subunit, TTC1386-protein, xanthine dehydrogenase, XM_{—}473199-protein, ycr102c-protein, YDL159W-A-protein, ygl237c-protein, YGR193C-protein, ygr221 c-protein, yhr131c-protein, yhr207c-protein, YKL038W-protein, YKL140W-protein, ylr065c-protein, ylr326w-protein, yml084w-protein, yol073c-protein, yol160w-protein, zinc finger protein, and zinc transporter, in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of citramalate, citrate, fumarate, glycerate, malate, oxalate, pyruvate, succinate and/or vitamin c and/or threonic acid or a composition comprising citramalate, citrate, fumarate, glycerate, malate, oxalate, pyruvate, succinate and/or vitamin c and/or threonic acid in said non-human organism or in the culture medium surrounding said non-human organism.

Item 2. A process for the production of a fine chemical selected from the group consisting of: citramalate, citrate, fumarate, glycerate, malate, oxalate, pyruvate, succinate and/or vitamin c and/or threonic acid, which comprises
(A)
   (i) increasing or generating of the expression of; and/or
   (ii) increasing or generating the expression of an expression product of; and/or
   (iii) increasing or generating one or more activities of an expression product encoded by;
   at least one respective nucleic acid molecule comprising a nucleic acid molecule selected from the group consisting of:
   (a) a nucleic acid molecule encoding the polypeptide shown in column 5 or 8 of Table II, preferably Table II B, application no.15, or a homolog or a fragment thereof;
   (b) a nucleic acid molecule shown in column 5 or 8 of Table I, preferably Table I B, application no.15, or a homolog or a fragment thereof (preferably the coding region thereof);
   (c) a nucleic acid molecule, which, as a result of the degeneracy of the genetic code, can be derived from a polypeptide sequence depicted in column 5 or 8 of Table II, preferably Table II B, application no.15;
   (d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 15, or the coding region thereof;
   (e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no.15;
   (i) a nucleic acid molecule encoding a polypeptide having the activity represented by a respective protein as depicted in column 5 of Table II, application no. 15;
   (j) a nucleic acid molecule which comprises a polynucleotide, which is obtained by amplifying a cDNA library or a genomic library using the primers as depicted in column 8 of Table III, application no. 15; and
   (k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
   or a nucleic acid molecule comprising a sequence which is complementary thereto; in a non-human organism or a part thereof, preferably a microorganism, a plant cell, a plant or a part thereof, as compared to a corresponding non-transformed wild type non-human organism or a part thereof; and
(B) growing the non-human organism or a part thereof under conditions which permit the production of citramalate, citrate, fumarate, glycerate, malate, oxalate, pyruvate, succinate and/or vitamin c and/or threonic acid or a composition comprising citramalate, citrate, fumarate, glycerate, malate, oxalate, pyruvate, succinate and/or vitamin c and/or threonic acid in said non-human organism or in the culture medium surrounding said non-human organism.

Item 3. A process of items 1 or 2, comprising of recovering citramalate, citrate, fumarate, glycerate, malate, oxalate, pyruvate, succinate and/or vitamin c and/or threonic acid in its free or bound form.

Item 4. The process of any one of items 1 to 3, comprising the folowing steps:
(a) selecting a non-human organism or a part thereof expressing a polypeptide encoded by the nucleic acid molecule characterized in item 2;
(b) mutagenizing the selected non-human organism or the part thereof;
(c) comparing the activity or the expression level of said polypeptide in the mutagenized non-human organism or the part thereof with the activity or the expression of said polypeptide of the selected non-human organisms or the part thereof;
(d) selecting the mutated non-human organisms or parts thereof, which comprise an increased activity or expression level of said polypeptide compared to the selected non-human organism or the part thereof;
(e) optionally, growing and cultivating the non-human organisms or the parts thereof; and
(f) recovering, and optionally isolating, the free or bound citramalate, citrate, fumarate, glycerate, malate, oxalate, pyruvate, succinate and/or vitamin c and/or threonic acid produced by the selected mutated non-human organisms or parts thereof.

Item 5. The process of any one of items 1 to 4 wherein the activity of said protein or the expression of said nucleic acid molecule is increased or generated transiently or stably.

Item 6. An isolated nucleic acid molecule comprising a nucleic acid molecule selected from the group consisting of:
(a) a nucleic acid molecule encoding a polypeptide shown in Table II, application no. 15, column 5 or 8, preferably shown in Table II B, application no. 15, column 8, or a homolog or a fragment thereof;
(b) a nucleic acid molecule shown in Table I, application no. 15, column 5 or 8, preferably shown in Table I B, application no. 15, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 15, preferably in column 8 of Table II B, application no. 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 8, preferably shown in column 8 of Table I B, application no. 15, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 15;
(i) a nucleic acid molecule encoding a polypeptide having the activity represented by a respective protein as depicted in column 5 of Table II, application no. 15;
(j) a nucleic acid molecule comprising a polynucleotide which is obtained by amplifying a cDNA library or a genomic library using the primers in Table III, application no. 15, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
or a nucleic acid molecule comprising a sequence which is complementary thereto.

Item 7. A nucleic acid construct which confers the expression of the nucleic acid molecule as defined in item 6, comprising one or more regulatory elements.

Item 8. An expression cassette comprising
(a) a promoter, preferably selected from the group consisting of Big35S, PCUbi, Super and USP;
(b) a nucleic acid molecule as defined in item6.

Item 9. A vector comprising the nucleic acid molecule as defined in item 6 or the nucleic acid construct as claimed in item 7 or the expression cassette as claimed in item 8.

Item 10. A host cell or a plant organelle, which has been transformed with the vector as claimed in item 9 or the nucleic acid molecule as defined in item 6 or the nucleic acid construct as claimed in item 7 or the expression cassette as claimed in item 8.

Item 11. The host cell of item 10, which is a plant cell or a microorganism.

Item 12. A process for producing a polypeptide, wherein the polypeptide is expressed in a host cell as claimed in item 10 or 11.

Item 13. A polypeptide produced by the process as claimed in item 12 or encoded by the nucleic acid molecule as defined in item 6.

Item 14. An antibody, which binds specifically to the polypeptide as claimed in item 13.

Item 15. A plant tissue, propagation material, harvested material or a plant comprising the host cell as claimed in item 10 or 11.

Item 16. A process for the identification of a compound conferring an increase in citramalate, citrate, fumarate, glycerate, malate, oxalate, pyruvate, succinate and/or vitamin c and/or threonic acid production in a non-human organism, comprising the steps:
(a) culturing a plant cell or tissue or microorganism or maintaining a plant expressing the polypeptide encoded by the nucleic acid molecule as defined in item 6 conferring an increase in the amount of citramalate, citrate, fumarate, glycerate, malate, oxalate, pyruvate, succinate and/or vitamin c and/or threonic acid in a non-human organism or a part thereof and a readout system capable of interacting with the polypeptide under suitable conditions which permit the interaction of the polypeptide with said readout system in the presence of a compound or a sample comprising a plurality of compounds and capable of providing a detectable signal in response to the binding of a compound to said polypeptide under conditions which permit the expression of said readout system and of the polypeptide encoded by the nucleic acid molecule of item 6 conferring an increase in the amount of citramalate, citrate, fumarate, glycerate, malate, oxalate, pyruvate, succinate and/or vitamin c and/or threonic acid in a non-human organism or a part thereof;
(b) identifying if the compound is an effective agonist by detecting the presence or absence or increase of a signal produced by said readout system.

Item 17. A method for the production of an agricultural composition comprising the steps of the method of item 16 and formulating the compound identified in item 16 in a form acceptable for an application in agriculture.

Item 18. A composition comprising the nucleic acid molecule as defined in item 6, the polypeptide as claimed in item 13, the nucleic acid construct as claimed in item 7, the expression cassette as claimed in item 8, the vector as claimed in item 9, the compound as claimed in item 16, the antibody as claimed in item 14, and optionally an agricultural acceptable carrier.

Item 19. Use of the nucleic acid molecule as defined in item 6 for the identification of a nucleic acid molecule conferring an increase in citramalate, citrate, fumarate, glycerate, malate, oxalate, pyruvate, succinate and/or vitamin c and/or threonic acid after expression.

Item 20. Cosmetic, pharmaceutical, food or feed composition comprising the nucleic acid moleculeas defined in item 6, the polypeptide as claimed in item 13, the nucleic acid construct as claimed in item 7, the expression cassette as claimed in item 8, the vector as claimed in item 9, the antibody as claimed in item 14 , the plant or plant tissue as claimed in item 15, the harvested material as claimed in item 15 or the host cell of claim as claimed in items 10 or 11.

Item 21. Use of the nuclei acid molecule as defined in item 6, the polypeptide as claimed in item 13, the nucleic acid construct as claimed in item 7, the expression cassette as claimed in item 8, the vector as claimed in item 9, the plant or plant tissue as claimed in item 15, or the host cell of item 10 to 11 for the production of plant resistant to a herbicide inhibiting the production of citramalate, citrate, fumarate, glycerate, malate, oxalate, pyruvate, succinate and/or vitamin c and/or threonic acid.

Item 22. A method for producing a plant with increased yield as compared to a corresponding non-transformed wild type plant, whereby the method comprises
(i) increasing or generating of the expression of; and/or
(ii) increasing or generating the expression of an expression product of; and/or
(iii) increasing or generating one or more activities of an expression product encoded by; at least one respective nucleic acid molecule comprising a nucleic acid molecule selected from the group consisting of:
(a) a nucleic acid molecule encoding the polypeptide shown in column 5 or 8 of Table II, preferably Table II B, application no. 15, or a homolog or a fragment thereof, in case in column 7 citramalate, citrate, fumarate, malate and/or succinate is indicated;
(b) a nucleic acid molecule shown in column 5 or 8 of Table I, preferably Table I B, application no. 15, or a homolog or a fragment thereof (preferably the coding region thereof) , in case in column 7 citramalate, citrate, fumarate, malate and/or succinate is indicated;
(c) a nucleic acid molecule, which, as a result of the degeneracy of the genetic code, can be derived from a polypeptide sequence depicted in column 5 or 8 of Table II, preferably Table II B, application no. 15, in case in column 7 citramalate, citrate, fumarate, malate and/or succinate is indicated;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 15, or the coding region thereof, in case in column 7 citramalate, citrate, fumarate, malate and/or succinate is indicated;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 15, in case in column 7 citramalate, citrate, fumarate, malate and/or succinate is indicated;
(i) a nucleic acid molecule encoding a polypeptide having the activity represented by a respective protein as depicted in column 5 of Table II, application no. 15, in case in column 7 citramalate, citrate, fumarate, malate and/or succinate is indicated;
(j) a nucleic acid molecule which comprises a polynucleotide, which is obtained by amplifying a cDNA library or a genomic library using the primers as depicted in column 8 of Table III, application no. 15, in case in column 7 citramalate, citrate, fumarate, malate and/or succinate is indicated; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto;
   in a plant cell, plant or a part thereof.

Item 23. A method for producing a plant with increased yield according to item 22, which comprises in addition the regeneration of a plant from such transformed plant cell, plant cell compartment, plant cell nucleus, plant cell or plant tissue with increased yield.

[0483.1.7.15] to [0492.1.7.15] for the disclosure of these paragraphs see [0483.1.7.7] to [0492.1.7.7] above

### Example 10

### Purification of a Fine Chemical e.g. Citramalate, Citrate, Fumarate, Glycerate, Malate, Oxalate, Pyruvate, Succinate and/or Vitamin C and/or Threonic Acid

The unambiguous detection for the presence of citramalate, citrate, fumarate, glycerate, malate, oxalate, pyruvate, succinate and/or vitamin c and/or threonic acid can be obtained by analyzing recombinant organisms using analytical standard methods: GC, GC-MS or TLC, as described (1997, in: Advances on Lipid Methodology, Fourth Edition: Christie, Oily Press, Dundee, 119-169; 1998, Gaschromatographie-Massenspektrometrie-Verfahren [Gas chromatography/mass spectrometric methods], Lipide 33:343-353).

The fine chemical citramalate, citrate, fumarate, glycerate, malate, oxalate, pyruvate, succinate and/or vitamin c and/or threonic acid produced in the organism can be recovered from cells or from the supernatant of the above-described culture by a variety of methods known in the art. For example, the culture supernatant is recovered first. To this end, the cells are harvested from the culture by slow centrifugation. Cells can generally be disrupted or lysed by standard techniques such as mechanical force or sonication. The cell debris is removed by centrifugation and the supernatant fraction, if appropriate together with the culture supernatant, is used for the further purification of the fine chemical citramalate, citrate, fumarate, glycerate, malate, oxalate, pyruvate, succinate and/or vitamin c and/or threonic acid. However, it is also possible to process the supernatant alone if the fine chemical citramalate, citrate, fumarate, glycerate, malate, oxalate, pyruvate, succinate and/or vitamin c and/or threonic acid are present in the supernatant in sufficiently high a concentration. In this case, the fine chemical citramalate, citrate, fumarate, glycerate, malate, oxalate, pyruvate, succinate and/or vitamin c and/or threonic acid, or mixture thereof, can be purified further for example via extraction and/or salt precipitation or via ion-exchange chromatography or as described above.

Identity and purity of the fine chemical citramalate, citrate, fumarate, glycerate, malate, oxalate, pyruvate, succinate and/or vitamin c and/or threonic acid isolated can be determined by standard techniques of the art. They encompass old techniques such as photometric methods and new techniques such as high-performance liquid chromatography (HPLC), gas chromatography (GC), spectroscopic methods, mass spectrometry (MS), staining methods, thin-layer chromatography, NIRS, enzyme assay or microbiological assays.

for the disclosure of this paragraph see [0494.1.7.7] above

### Example 11a

### PCR Amplification of the Sequences

Unless otherwise specified, standard methods as described in Sambrook et al., Molecular Cloning: A laboratory manual, Cold Spring Harbor 1989, Cold Spring Harbor Laboratory Press are used.

The inventive sequences as shown in the respective line in Table I, column 5, preferably the coding region thereof, were amplified by PCR as described in the protocol of the Pfu Ultra, Pfu Turbo or Herculase DNA polymerase (Stratagene). The composition for the protocol of the Pfu Ultra, Pfu Turbo or Herculase DNA polymerase was as follows: 1×PCR buffer (Stratagene), 0.2 mM of each dNTP, 100 ng genomic DNA of Saccharomyces cerevisiae (strain S288C; Research Genetics, Inc., now Invitrogen), Escherichia coli (strain MG1655; E. coli Genetic Stock Center), Synechocystis sp. (strain PCC6803), Azotobacter vinelandii (strain N.R. Smith,16), Thermus thermophilus (H B8) or 50 ng cDNA from various tissues and development stages of Arabidopsis thaliana (ecotype Columbia), Physcomitrella patens, Glycine max (variety Resnick), Brassica napus, Oryza sativa or Zea mays (variety B73, Mo17, A188), 50 pmol forward primer, 50 pmol reverse primer, with or without 1 M Betaine, 2.5 u Pfu Ultra, Pfu Turbo or Herculase DNA polymerase.

The amplification cycles were as follows:

1 cycle of 2-3 minutes at 94-95° C., then 25-36 cycles with 30-60 seconds at 94-95° C., 30-45 seconds at 50-60° C. and 210-480 seconds at 72° C., followed by 1 cycle of 5-10 minutes at 72° C., then 4-16° C.—preferably for Saccharomyces cerevisiae, Escherichia coli, Synechocystis sp., Azotobacter vinelandii, Thermus thermophilus.

In case of Arabidopsis thaliana, Brassica napus, Glycine max, Oryza sativa, Physcomitrella patens, Zea mays the amplification cycles were as follows:

1 cycle with 30 seconds at 94° C., 30 seconds at 61° C., 15 minutes at 72° C., then 2 cycles with 30 seconds at 94° C., 30 seconds at 60° C., 15 minutes at 72° C., then 3 cycles with 30 seconds at 94° C., 30 seconds at 59° C., 15 minutes at 72° C., then 4 cycles with 30 seconds at 94° C., 30 seconds at 58° C., 15 minutes at 72° C., then 25 cycles with 30 seconds at 94° C., 30 seconds at 57° C., 15 minutes at 72° C., then 1 cycle with 10 minutes at 72° C., then finally 4-16° C.

RNAs were generated with the RNeasy Plant Kit according to the standard protocol (Qiagen) and Superscript II Reverse Transkriptase was used to produce double stranded cDNA according to the standard protocol (Invitrogen).

ORF specific primer pairs for the genes to be expressed are shown in the respective line in Table III, column 8. The following adapter sequences were added to Saccharomyces cerevisiae ORF specific primers (see Table III) for cloning purposes:

SEQ ID NO: 20

| | | |
|---|---|---|
| i) | foward primer: | 5′-GGAATTCCAGCTGACCACC-3′ |

SEQ ID NO: 21

| | | |
|---|---|---|
| ii) | reverse primer: | 5′-GATCCCCGGGAATTGCCATG-3′ |

These adaptor sequences allow cloning of the ORF into the various vectors containing the Resgen adaptors, see table column E of Table c.

The following adapter sequences were added to Saccharomyces cerevisiae, Escherichia coli, Synechocystis sp., Azotobacter vinelandii, Thermus thermophilus, Arabidopsis thaliana, Brassica napus, Glycine max, Oryza sativa , Physcomitrella patens, or Zea mays ORF specific primers for cloning purposes:

| SEQ ID NO: 22 | | |
|---|---|---|
| iii) | forward primer: | 5′-TTGCTCTTCC-3′ |
| SEQ ID NO: 23 | | |
| iiii) | reverse primer: | 5′-TTGCTCTTCG-3′ |

The adaptor sequences allow cloning of the ORF into the various vectors containing the Colic adaptors, see table column E of Table c.

Therefore for amplification and cloning of Saccharomyces cerevisiae SEQ ID NO: 64177, a primer consisting of the adaptor sequence i) and the ORF specific sequence SEQ ID NO: 64193 and a second primer consisting of the adaptor sequence ii) and the ORF specific sequence SEQ ID NO: 64194 were used.

For amplification and cloning of Escherichia coli SEQ ID NO: 7333, a primer consisting of the adaptor sequence iii) and the ORF specific sequence SEQ ID NO: 7679 and a second primer consisting of the adaptor sequence iiii) and the ORF specific sequence SEQ ID NO: 7680 were used.

For amplification and cloning of Synechocystis sp. SEQ ID NO: 11990, a primer consisting of the adaptor sequence iii) and the ORF specific sequence SEQ ID NO: 12064 and a second primer consisting of the adaptor sequence iiii) and the ORF specific sequence SEQ ID NO: 12065 were used.

For amplification and cloning of Arabidopsis thaliana SEQ ID NO: 812, a primer consisting of the adaptor sequence iii) and the ORF specific sequence SEQ ID NO: 1054 and a second primer consisting of the adaptor sequence iiii) and the ORF specific sequence SEQ ID NO: 1055 were used.

For amplification and cloning of Azotobacter vinelandii SEQ ID NO: 6040, a primer consisting of the adaptor sequence iii) and the ORF specific sequence SEQ ID NO: 6070 and a second primer consisting of the adaptor sequence iiii) and the ORF specific sequence SEQ ID NO: 6071 were used.

For amplification and cloning of Brassica napus SEQ ID NO: 15532, a primer consisting of the adaptor sequence iii) and the ORF specific sequence SEQ ID NO: 16150 and a second primer consisting of the adaptor sequence iiii) and the ORF specific sequence SEQ ID NO: 16151 were used.

For amplification and cloning of Glycine max SEQ ID NO: 69, a primer consisting of the adaptor sequence iii) and the ORF specific sequence SEQ ID NO: 343 and a second primer consisting of the adaptor sequence iiii) and the ORF specific sequence SEQ ID NO: 344 were used.

For amplification and cloning of Oryza sativa SEQ ID NO: 34301, a primer consisting of the adaptor sequence iii) and the ORF specific sequence SEQ ID NO: 34595 and a second primer consisting of the adaptor sequence iiii) and the ORF specific sequence SEQ ID NO: 34596 were used.

For amplification and cloning of Thermus thermophilus SEQ ID NO: 12698 , a primer consisting of the adaptor sequence iii) and the ORF specific sequence SEQ ID NO: 12970 and a second primer consisting of the adaptor sequence iiii) and the ORF specific sequence SEQ ID NO: 12971 were used.

Following these examples every sequence disclosed in Table I, preferably column 5, especially the coding region thereof can be cloned by fusing the adaptor sequences to the respective specific primers sequences as disclosed in Table III, column 8 using the respective vectors shown in Table c.

[0496.1.7.15] to [0499.1.7.15] for the disclosure of these paragraphs see [0496.1.7.7] to [0499.1.7.7] above

### Example 11e

### Cloning of Inventive Sequences as Shown in Table I, Column 5 in the Different Expression Vectors

For cloning of for example SEQ ID NO: 7333 from Escherichia coli or any other ORF from Escherichia coli, Synechocystis sp., Azotobacter vinelandii, Thermus thermophilus, Arabidopsis thaliana, Brassica napus, Glycine max, or Oryza sativa the vector DNA was treated with the restriction enzymes Pacl and Ncol following the standard protocol (MBI Fermentas).

For cloning for example the ORFs of SEQ ID NO: 64177 from Saccharomyces cerevisiae or any other ORF from Saccharomyces cerevisiae into vectors containing the Resgen adaptor sequence the respective vector DNA was treated with the restriction enzyme Ncol.

For cloning of for example ORFs SEQ ID NO: 63334 from Saccharomyces cerevisiae or any other ORF from Saccharomyces cerevisiae into vectors containing the Colic adaptor sequence, the respective vector DNA was treated with the restriction enzymes Pacl and Ncol following the standard protocol (MBI Fermentas).

In all cases the reaction was stopped by inactivation at 70° C. for 20 minutes and purified over QlAquick or NucleoSpin Extract II columns following the standard protocol (Qiagen or Macherey-Nagel).

Then the PCR-product representing the amplified ORF with the respective adapter sequences and the vector DNA were treated with T4 DNA polymerase according to the standard protocol (MBI Fermentas) to produce single stranded overhangs with the parameters 1 unit T4 DNA polymerase at 37° C. for 2-10 minutes for the vector and 1-2 u T4 DNA polymerase at 15-17° C. for 10-60 minutes for the PCR product representing SEQ ID NO: 7333.

The reaction was stopped by addition of high-salt buffer and purified over QlAquick or NucleoSpin Extract II columns following the standard protocol (Qiagen or Macherey-Nagel).

According to this example the skilled person is able to clone all sequences disclosed in Table I, preferably column 5 or column 8, especially the coding region thereof.

Approximately 30-60 ng of prepared vector and a defined amount of prepared amplificate were mixed and hybridized at 65° C. for 15 minutes followed by 37° C. 0.1° C/1 seconds, followed by 37° C. 10 minutes, followed by 0.1° C/1 seconds, then 4-10° C.

The ligated constructs were transformed in the same reaction vessel by addition of competent E. coli cells (strain DHSalpha) and incubation for 20 minutes at 1° C. followed by a heat shock for 90 seconds at 42° C. and cooling to 1-4° C. Then, complete medium (SOC) was added and the mixture was incubated for 45 minutes at 37° C. The entire mixture was subsequently plated onto an agar plate with 0.05 mg/ml kanamycin and incubated overnight at 37° C.

The outcome of the cloning step was verified by amplification with the aid of primers which bind upstream and downstream of the integration site, thus allowing the amplification of the insertion. The amplifications were carried out as described in the protocol of Taq DNA polymerase (Gibco-BRL).

The amplification cycles were as follows:

1 cycle of 1-5 minutes at 94° C., followed by 35 cycles of in each case 15-60 seconds at 94° C., 15-60 seconds at 50-66° C. and 5-15 minutes at 72° C., followed by 1 cycle of 10 minutes at 72° C., then 4-16° C.

Several colonies were checked, but only one colony for which a PCR product of the expected size was detected was used in the following steps.

A portion of this positive colony was transferred into a reaction vessel filled with complete medium (LB) supplemented with kanamycin and incubated overnight at 37° C.

The plasmid preparation was carried out as specified in the Qiaprep or NucleoSpin Multi-96 Plus standard protocol (Qiagen or Macherey-Nagel).

[0501.1.7.15] to [0503.1.7.15] for the disclosure of these paragraphs see [0501.1.7.7] to [0503.1.7.7] above

Table d showing results of plant analyses

TABLE d

| SeqID | Target | Locus | Metabolite | Source | Promotor | Method | Min | Max |
|---|---|---|---|---|---|---|---|---|
| 17451 | non-targ | At1g07430 | succinate | ARA_LEAF | p-PcUBI | GC | 47 | 67 |
| 133001 | non-targ | At1g13930 | malate | ARA_LEAF | p-PcUBI | GC | 82 | 90 |
| 133001 | non-targ | At1g13930 | succinate | ARA_LEAF | p-PcUBI | LC | 33 | 79 |
| 17968 | non-targ | At1g36730 | malate | ARA_LEAF | p-PcUBI | GC | 65 | 137 |
| 17968 | non-targ | At1g36730 | vitc a/o thra | ARA_LEAF | p-PcUBI | GC | 31 | 56 |
| 18070 | non-targ | At1g43850 | fumarate | ARA_LEAF | p-PcUBI | GC | 63 | 108 |
| 812 | non-targ | At1g48040 | fumarate | ARA_LEAF | p-PcUBI | LC | 49 | 81 |
| 19419 | non-targ | At1g72770 | vitc a/o thra | ARA_LEAF | p-PcUBI | GC | 31 | 109 |
| 19919 | non-targ | At2g30360 | fumarate | ARA_LEAF | p-PcUBI | GC | 75 | 420 |
| 19919 | non-targ | At2g30360 | malate | ARA_LEAF | p-PcUBI | GC | 52 | 364 |
| 19919 | non-targ | At2g30360 | succinate | ARA_LEAF | p-PcUBI | GC | 24 | 75 |
| 133042 | non-targ | At2g37040 | fumarate | ARA_LEAF | p-PcUBI | GC | 70 | 89 |
| 133349 | non-targ | At2g41680 | succinate | ARA_LEAF | p-PcUBI | LC | 34 | 91 |
| 120810 | plastidic | At2g44350 | succinate | ARA_LEAF | p-Super | LC | 46 | 132 |
| 21497 | plastidic | At3g04050 | citramalate | ARA_LEAF | p-PcUBI | GC | 49 | 721 |
| 21497 | plastidic | At3g04050 | succinate | ARA_LEAF | p-PcUBI | GC | 25 | 537 |
| 22015 | non-targ | At3g06270 | malate | ARA_LEAF | p-PcUBI | GC | 63 | 203 |
| 22015 | non-targ | At3g06270 | succinate | ARA_LEAF | p-PcUBI | GC | 24 | 55 |
| 22249 | non-targ | At3g08710 | fumarate | ARA_LEAF | p-PcUBI | GC | 72 | 165 |
| 22249 | non-targ | At3g08710 | malate | ARA_LEAF | p-PcUBI | GC | 62 | 162 |
| 130491 | non-targ | At3g15360 | glycerate | ARA_LEAF | p-PcUBI | GC | 31 | 129 |
| 2367 | non-targ | At3g61830 | fumarate | ARA_LEAF | p-PcUBI | GC | 78 | 248 |
| 2367 | non-targ | At3g61830 | malate | ARA_LEAF | p-PcUBI | GC | 93 | 115 |
| 2573 | non-targ | At3g62930 | citramalate | ARA_LEAF | p-PcUBI | GC | 70 | 163 |
| 2573 | non-targ | At3g62930 | fumarate | ARA_LEAF | p-PcUBI | GC | 62 | 222 |
| 2573 | non-targ | At3g62930 | glycerate | ARA_LEAF | p-PcUBI | GC | 35 | 101 |
| 2573 | non-targ | At3g62930 | malate | ARA_LEAF | p-PcUBI | GC | 57 | 206 |
| 2573 | non-targ | At3g62930 | succinate | ARA_LEAF | p-PcUBI | LC | 30 | 137 |
| 2573 | non-targ | At3g62930 | vitc a/o thra | ARA_LEAF | p-PcUBI | GC | 51 | 96 |
| 2935 | non-targ | At3g62950 | citramalate | ARA_LEAF | p-PcUBI | GC | 65 | 235 |
| 2935 | non-targ | At3g62950 | fumarate | ARA_LEAF | p-PcUBI | GC | 64 | 136 |
| 2935 | non-targ | At3g62950 | glycerate | ARA_LEAF | p-PcUBI | GC | 31 | 113 |
| 2935 | non-targ | At3g62950 | malate | ARA_LEAF | p-PcUBI | GC | 100 | 274 |
| 2935 | non-targ | At3g62950 | succinate | ARA_LEAF | p-PcUBI | LC | 30 | 257 |
| 133541 | non-targ | At4g03520 | glycerate | ARA_LEAF | p-PcUBI | GC | 35 | 112 |
| 23482 | non-targ | At4g15660 | citramalate | ARA_LEAF | p-PcUBI | GC | 71 | 156 |
| 23482 | non-targ | At4g15660 | fumarate | ARA_LEAF | p-PcUBI | GC | 195 | 463 |
| 23482 | non-targ | At4g15660 | glycerate | ARA_LEAF | p-PcUBI | GC | 41 | 158 |
| 23482 | non-targ | At4g15660 | malate | ARA_LEAF | p-PcUBI | GC | 123 | 321 |
| 23482 | non-targ | At4g15660 | succinate | ARA_LEAF | p-PcUBI | LC | 133 | 346 |
| 23482 | non-targ | At4g15660 | vitc a/o thra | ARA_LEAF | p-PcUBI | GC | 29 | 89 |
| 3279 | non-targ | At4g15670 | fumarate | ARA_LEAF | p-PcUBI | GC | 196 | 830 |
| 3279 | non-targ | At4g15670 | glycerate | ARA_LEAF | p-PcUBI | GC | 49 | 224 |
| 3279 | non-targ | At4g15670 | malate | ARA_LEAF | p-PcUBI | GC | 117 | 935 |
| 3279 | non-targ | At4g15670 | succinate | ARA_LEAF | p-PcUBI | LC | 77 | 314 |
| 23844 | non-targ | At4g15690 | citramalate | ARA_LEAF | p-PcUBI | GC | 47 | 204 |
| 23844 | non-targ | At4g15690 | fumarate | ARA_LEAF | p-PcUBI | GC | 66 | 427 |
| 23844 | non-targ | At4g15690 | malate | ARA_LEAF | p-PcUBI | GC | 76 | 386 |
| 23844 | non-targ | At4g15690 | succinate | ARA_LEAF | p-PcUBI | GC | 32 | 158 |
| 23844 | non-targ | At4g15690 | vitc a/o thra | ARA_LEAF | p-PcUBI | GC | 30 | 86 |
| 3654 | non-targ | At4g15700 | glycerate | ARA_LEAF | p-PcUBI | GC | 48 | 198 |
| 68849 | non-targ | At4g26080 | citramalate | ARA_LEAF | p-PcUBI | GC | 47 | 70 |
| 68849 | non-targ | At4g26080 | succinate | ARA_LEAF | p-PcUBI | LC | 30 | 52 |
| 4102 | non-targ | At4g33040 | citramalate | ARA_LEAF | p-PcUBI | GC | 43 | 136 |
| 4102 | non-targ | At4g33040 | fumarate | ARA_LEAF | p-PcUBI | GC | 72 | 537 |
| 4102 | non-targ | At4g33040 | malate | ARA_LEAF | p-PcUBI | GC | 65 | 296 |
| 4102 | non-targ | At4g33040 | succinate | ARA_LEAF | p-PcUBI | LC | 42 | 149 |
| 4102 | non-targ | At4g33040 | vitc a/o thra | ARA_LEAF | p-PcUBI | GC | 32 | 102 |
| 4348 | non-targ | At4g35310 | malate | ARA_LEAF | p-PcUBI | GC | 70 | 296 |
| 24438 | non-targ | At5g03720 | fumarate | ARA_LEAF | p-PcUBI | GC | 66 | 1135 |
| 24438 | non-targ | At5g03720 | malate | ARA_LEAF | p-PcUBI | GC | 133 | 687 |
| 24438 | non-targ | At5g03720 | succinate | ARA_LEAF | p-PcUBI | GC | 30 | 376 |
| 24492 | non-targ | At5g07200 | glycerate | ARA_LEAF | p-PcUBI | GC | 33 | 74 |
| 24492 | non-targ | At5g07200 | malate | ARA_LEAF | p-PcUBI | GC | 52 | 329 |
| 4904 | non-targ | At5g18600 | succinate | ARA_LEAF | p-PcUBI | LC | 39 | 132 |
| 25344 | non-targ | At5g27640 | malate | ARA_LEAF | p-PcUBI | GC | 52 | 173 |
| 25428 | non-targ | At5g39760 | fumarate | ARA_LEAF | p-PcUBI | LC | 54 | 110 |
| 25428 | non-targ | At5g39760 | malate | ARA_LEAF | p-PcUBI | GC | 52 | 175 |
| 25498 | non-targ | At5g59220 | succinate | ARA_LEAF | p-PcUBI | LC | 30 | 67 |
| 70038 | plastidic | At5g63680 | succinate | ARA_LEAF | p-PcUBI | GC | 31 | 226 |
| 5493 | non-targ | At5g64920 | fumarate | ARA_LEAF | p-PcUBI | GC | 81 | 158 |
| 70583 | non-targ | Avin- | citramalate | ARA_LEAF | p-PcUBI | GC | 41 | 106 |
| | | DRAFT-_0035 | | | | | | |
| 70583 | non-targ | Avin- | fumarate | ARA_LEAF | p-PcUBI | LC | 63 | 130 |
| | | DRAFT-_0035 | | | | | | |
| 70583 | non-targ | Avin- | malate | ARA_LEAF | p-PcUBI | GC | 55 | 281 |
| | | DRAFT-_0035 | | | | | | |
| 70715 | non-targ | AVIN- | malate | ARA_LEAF | p-PcUBI | GC | 52 | 146 |
| | | DRAFT-_0539 | | | | | | |
| 25676 | non-targ | Avin- | malate | ARA_LEAF | p-PcUBI | GC | 64 | 122 |
| | | DRAFT-_1045 | | | | | | |
| 25780 | non-targ | Avin- | malate | ARA_LEAF | p-PcUBI | GC | 54 | 91 |
| | | DRAFT-_1398 | | | | | | |
| 25780 | non-targ | Avin- | succinate | ARA_LEAF | p-PcUBI | LC | 32 | 90 |
| | | DRAFT-_1398 | | | | | | |
| 26196 | non-targ | Avin- | fumarate | ARA_LEAF | p-PcUBI | GC | 66 | 110 |
| | | DRAFT-_1624 | | | | | | |
| 26196 | non-targ | Avin- | malate | ARA_LEAF | p-PcUBI | GC | 67 | 120 |
| | | DRAFT-_1624 | | | | | | |
| 133719 | non-targ | Avin- | fumarate | ARA_LEAF | p-PcUBI | GC | 148 | 507 |
| | | DRAFT-_1699 | | | | | | |
| 26434 | non-targ | Avin- | succinate | ARA_LEAF | p-PcUBI | LC | 51 | 154 |
| | | DRAFT-_1806 | | | | | | |
| 114198 | non-targ | Avin- | succinate | ARA_LEAF | p-PcUBI | LC | 30 | 43 |
| | | DRAFT-_2010 | | | | | | |
| 6040 | non-targ | Avin- | glycerate | ARA_LEAF | p-PcUBI | GC | 31 | 81 |
| | | DRAFT-_2091 | | | | | | |
| 71325 | non-targ | Avin- | succinate | ARA_LEAF | p-PcUBI | LC | 45 | 203 |
| | | DRAFT-_2365 | | | | | | |
| 71763 | non-targ | Avin- | citramalate | ARA_LEAF | p-PcUBI | GC | 41 | 49 |
| | | DRAFT-_2369 | | | | | | |
| 28040 | non-targ | Avin- | malate | ARA_LEAF | p-PcUBI | GC | 121 | 517 |
| | | DRAFT-_2754 | | | | | | |
| 6075 | non-targ | Avin- | citramalate | ARA_LEAF | p-PcUBI | GC | 47 | 117 |
| | | DRAFT-_3028 | | | | | | |
| 6075 | non-targ | Avin- | fumarate | ARA_LEAF | p-PcUBI | GC | 98 | 406 |
| | | DRAFT-_3028 | | | | | | |
| 6075 | non-targ | Avin- | malate | ARA_LEAF | p-PcUBI | GC | 57 | 323 |
| | | DRAFT-_3028 | | | | | | |
| 6075 | non-targ | Avin- | succinate | ARA_LEAF | p-PcUBI | LC | 97 | 190 |
| | | DRAFT-_3028 | | | | | | |
| 6075 | non-targ | Avin- | vitc a/o thra | ARA_LEAF | p-PcUBI | GC | 31 | 137 |
| | | DRAFT-_3028 | | | | | | |
| 29246 | non-targ | Avin- | citramalate | ARA_LEAF | p-PcUBI | GC | 41 | 84 |
| | | DRAFT-_3186 | | | | | | |
| 29246 | non-targ | Avin- | fumarate | ARA_LEAF | p-PcUBI | GC | 100 | 236 |
| | | DRAFT-_3186 | | | | | | |
| 29500 | non-targ | Avin- | succinate | ARA_LEAF | p-PcUBI | LC | 29 | 139 |
| | | DRAFT-_3253 | | | | | | |
| 31026 | non-targ | Avin- | fumarate | ARA_LEAF | p-PcUBI | GC | 61 | 240 |
| | | DRAFT-_3605 | | | | | | |
| 31026 | non-targ | Avin- | malate | ARA_LEAF | p-PcUBI | GC | 53 | 314 |
| | | DRAFT-_3605 | | | | | | |
| 31026 | non-targ | Avin- | succinate | ARA_LEAF | p-PcUBI | GC | 66 | 203 |
| | | DRAFT-_3605 | | | | | | |
| 114231 | non-targ | Avin- | succinate | ARA_LEAF | p-PcUBI | LC | 33 | 291 |
| | | DRAFT-_3629 | | | | | | |
| 31717 | non-targ | Avin- | malate | ARA_LEAF | p-PcUBI | GC | 73 | 137 |
| | | DRAFT-_4384 | | | | | | |
| 72935 | non-targ | Avin- | fumarate | ARA_LEAF | p-PcUBI | LC | 57 | 91 |
| | | DRAFT-_4385 | | | | | | |
| 72935 | non-targ | Avin- | malate | ARA_LEAF | p-PcUBI | GC | 82 | 144 |
| | | DRAFT-_4385 | | | | | | |
| 31926 | non-targ | Avin- | malate | ARA_LEAF | p-PcUBI | GC | 59 | 109 |
| | | DRAFT-_4562 | | | | | | |
| 134171 | non-targ | Avin- | succinate | ARA_LEAF | p-PcUBI | LC | 33 | 110 |
| | | DRAFT-_4594 | | | | | | |
| 73719 | non-targ | Avin- | fumarate | ARA_LEAF | p-PcUBI | LC | 52 | 113 |
| | | DRAFT-_4606 | | | | | | |
| 73719 | non-targ | Avin- | malate | ARA_LEAF | p-PcUBI | GC | 70 | 81 |
| | | DRAFT-_4606 | | | | | | |
| 74452 | non-targ | Avin- | malate | ARA_LEAF | p-PcUBI | GC | 59 | 107 |
| | | DRAFT-_4989 | | | | | | |
| 6510 | non-targ | Avin- | malate | ARA_LEAF | p-PcUBI | GC | 74 | 412 |
| | | DRAFT-_5103 | | | | | | |
| 32037 | non-targ | Avin- | malate | ARA_LEAF | p-PcUBI | GC | 181 | 293 |
| | | DRAFT-_5246 | | | | | | |
| 74729 | non-targ | Avin- | succinate | ARA_LEAF | p-PcUBI | GC | 26 | 107 |
| | | DRAFT-_6679 | | | | | | |
| 34301 | non-targ | AX653549 | fumarate | ARA_LEAF | p-PcUBI | GC | 92 | 266 |
| 34301 | non-targ | AX653549 | malate | ARA_LEAF | p-PcUBI | GC | 62 | 106 |
| 34602 | non-targ | AY087308 | fumarate | ARA_LEAF | p-PcUBI | GC | 84 | 224 |
| 34602 | non-targ | AY087308 | malate | ARA_LEAF | p-PcUBI | GC | 51 | 252 |
| 34602 | non-targ | AY087308 | succinate | ARA_LEAF | p-PcUBI | LC | 33 | 109 |
| 136120 | non-targ | Bab08741 | fumarate | ARA_LEAF | p-PcUBI | GC | 165 | 582 |
| 34889 | plastidic | B0004 | malate | ARA_LEAF | p-Super | GC | 61 | 169 |
| 134299 | non-targ | B0063 | fumarate | ARA_LEAF | p-Super | GC | 63 | 529 |
| 134299 | non-targ | B0063 | succinate | ARA_LEAF | p-Super | LC | 39 | 119 |
| 35482 | non-targ | B0124 | citrate | ARA_SEED_2 | p-USP | GC | 91 | 837 |
| 35590 | non-targ | B0221 | fumarate | ARA_LEAF | p-Super | LC | 49 | 70 |
| 121111 | non-targ | B0312 | citrate | ARA_SEED_2 | p-USP | LC | 126 | 165 |
| 7333 | non-targ | B0449 | malate | ARA_LEAF | p-Super | GC | 94 | 471 |
| 7686 | non-targ | B0486 | malate | ARA_LEAF | p-Super | GC | 61 | 99 |
| 7686 | non-targ | B0486 | succinate | ARA_LEAF | p-Super | LC | 31 | 56 |
| 134402 | non-targ | B0514 | citrate | ARA_SEED_2 | p-USP | LC | 88 | 115 |
| 35967 | plastidic | B0593 | fumarate | ARA_LEAF | p-Super | GC | 130 | 365 |
| 35967 | plastidic | B0593 | malate | ARA_LEAF | p-Super | GC | 168 | 536 |
| 36114 | non-targ | B0752 | fumarate | ARA_LEAF | p-Super | GC | 158 | 628 |
| 75807 | non-targ | B0801 | succinate | ARA_LEAF | p-Super | LC | 67 | 135 |
| 36489 | non-targ | B0885 | malate | ARA_LEAF | p-Super | GC | 134 | 141 |
| 36670 | non-targ | B0963 | vitc a/o thra | ARA_LEAF | p-Super | GC | 43 | 81 |
| 7941 | non-targ | B1003 | malate | ARA_LEAF | p-Super | GC | 107 | 1076 |
| 37483 | non-targ | B1255 | malate | ARA_LEAF | p-Super | GC | 50 | 119 |
| 134655 | non-targ | B1285 | succinate | ARA_LEAF | p-Super | LC | 31 | 37 |
| 134697 | non-targ | B1390 | malate | ARA_LEAF | p-Super | GC | 60 | 182 |
| 134756 | non-targ | B1425 | malate | ARA_LEAF | p-Super | GC | 61 | 256 |
| 38266 | plastidic | B1431 | succinate | ARA_LEAF | p-Super | LC | 29 | 44 |
| 38289 | non-targ | B1445 | citramalate | ARA_LEAF | p-Super | GC | 67 | 102 |
| 7947 | non-targ | B1522 | citramalate | ARA_LEAF | p-Super | GC | 41 | 92 |
| 7947 | non-targ | B1522 | malate | ARA_LEAF | p-Super | GC | 52 | 311 |
| 78154 | non-targ | B1589 | fumarate | ARA_LEAF | p-Super | GC | 117 | 167 |
| 115064 | plastidic | B1611 | fumarate | ARA_LEAF | p-Super | GC | 187 | 514 |
| 38345 | non-targ | B1627 | succinate | ARA_LEAF | p-Super | GC | 35 | 56 |
| 78265 | non-targ | B1651 | succinate | ARA_LEAF | p-Super | GC | 27 | 37 |
| 78753 | non-targ | B1670 | citramalate | ARA_LEAF | p-Super | GC | 49 | 89 |
| 78753 | non-targ | B1670 | malate | ARA_LEAF | p-Super | GC | 60 | 138 |
| 78771 | non-targ | B1672 | citramalate | ARA_LEAF | p-Super | GC | 43 | 84 |
| 78771 | non-targ | B1672 | malate | ARA_LEAF | p-Super | GC | 79 | 152 |
| 78771 | non-targ | B1672 | vitc a/o thra | ARA_LEAF | p-Super | GC | 30 | 48 |
| 134762 | non-targ | B1742 | malate | ARA_LEAF | p-Super | GC | 48 | 86 |
| 134762 | non-targ | B1742 | succinate | ARA_LEAF | p-Super | LC | 33 | 52 |
| 78779 | plastidic | B1747 | succinate | ARA_SEED_2 | p-USP | LC | 51 | 158 |
| 78852 | non-targ | B1755 | vitc a/o thra | ARA_LEAF | p-Super | GC | 30 | 46 |
| 38573 | non-targ | B1845 | malate | ARA_LEAF | p-Super | GC | 92 | 424 |
| 134783 | non-targ | B1865 | vitc a/o thra | ARA_SEED_2 | p-USP | GC | 38 | 301 |
| 38767 | non-targ | B1898 | succinate | ARA_LEAF | p-Super | LC | 35 | 94 |
| 134864 | non-targ | B1938 | fumarate | ARA_LEAF | p-Super | GC | 71 | 324 |
| 134864 | non-targ | B1938 | malate | ARA_LEAF | p-Super | GC | 62 | 332 |
| 134864 | non-targ | B1938 | succinate | ARA_LEAF | p-Super | GC | 41 | 171 |
| 79217 | non-targ | B2027 | malate | ARA_LEAF | p-Super | GC | 77 | 186 |
| 39237 | non-targ | B2399 | fumarate | ARA_LEAF | p-Super | GC | 190 | 3230 |
| 39237 | non-targ | B2399 | succinate | ARA_LEAF | p-Super | LC | 86 | 421 |
| 39237 | non-targ | B2399 | vitc a/o thra | ARA_LEAF | p-Super | GC | 45 | 235 |
| 39300 | non-targ | B2414 | vitc a/o thra | ARA_LEAF | p-Super | GC | 30 | 46 |
| 134924 | plastidic | B2421 | vitc a/o thra | ARA_SEED_2 | p-USP | GC | 38 | 197 |
| 40726 | non-targ | B2634 | fumarate | ARA_LEAF | p-Super | GC | 134 | 861 |
| 40726 | non-targ | B2634 | malate | ARA_LEAF | p-Super | GC | 139 | 511 |
| 80756 | non-targ | B2701 | succinate | ARA_LEAF | p-Super | GC | 33 | 60 |
| 81075 | non-targ | B2747 | succinate | ARA_LEAF | p-Super | GC | 33 | 53 |
| 81576 | non-targ | B2818 | succinate | ARA_LEAF | p-Super | GC | 30 | 59 |
| 115864 | non-targ | B2866 | succinate | ARA_LEAF | p-Super | GC | 26 | 34 |
| 135893 | non-targ | B3008 | succinate | ARA_LEAF | p-Super | GC | 26 | 63 |
| 42502 | non-targ | B3262 | fumarate | ARA_LEAF | p-Super | LC | 69 | 208 |
| 42502 | non-targ | B3262 | malate | ARA_LEAF | p-Super | GC | 76 | 135 |
| 136077 | non-targ | B3624 | fumarate | ARA_LEAF | p-Super | GC | 69 | 1749 |
| 136098 | non-targ | B3777 | succinate | ARA_LEAF | p-Super | LC | 29 | 54 |
| 83726 | non-targ | B3791 | malate | ARA_LEAF | p-Super | GC | 52 | 264 |
| 83726 | non-targ | B3791 | succinate | ARA_LEAF | p-Super | LC | 29 | 160 |
| 44196 | non-targ | B3814 | citramalate | ARA_LEAF | p-Super | GC | 41 | 45 |
| 10708 | non-targ | B3817 | fumarate | ARA_LEAF | p-Super | GC | 122 | 198 |
| 10726 | non-targ | B3823 | citramalate | ARA_LEAF | p-Super | GC | 44 | 151 |
| 10726 | non-targ | B3823 | malate | ARA_LEAF | p-Super | GC | 56 | 84 |
| 44372 | non-targ | B3989 | succinate | ARA_LEAF | p-Super | LC | 53 | 90 |
| 44378 | non-targ | B4012 | fumarate | ARA_SEED_2 | p-USP | GC | 58 | 204 |
| 10740 | non-targ | B4029 | fumarate | ARA_LEAF | p-Super | GC | 76 | 467 |
| 10740 | non-targ | B4029 | malate | ARA_LEAF | p-Super | GC | 81 | 511 |
| 10740 | non-targ | B4029 | succinate | ARA_LEAF | p-Super | GC | 29 | 444 |
| 106532 | non-targ | B4050 | fumarate | ARA_LEAF | p-Super | GC | 127 | 1497 |
| 106532 | non-targ | B4050 | malate | ARA_LEAF | p-Super | GC | 52 | 553 |
| 94542 | non-targ | B4056 | citramalate | ARA_LEAF | p-Super | GC | 41 | 94 |
| 45022 | non-targ | B4139 | citrate | ARA_SEED_2 | p-USP | GC | 119 | 342 |
| 45321 | non-targ | B4256 | fumarate | ARA_LEAF | p-Super | LC | 40 | 45 |
| 45321 | non-targ | B4256 | malate | ARA_LEAF | p-Super | GC | 201 | 278 |
| 45556 | non-targ | B4384 | vitc a/o thra | ARA_SEED_2 | p-USP | GC | 36 | 222 |
| 84079 | plastidic | D90900 | succinate | ARA_LEAF | p-PcUBI | LC | 33 | 49 |
| 10811 | non-targ | GM02LC12622 | succinate | ARA_LEAF | p-PcUBI | LC | 32 | 137 |
| 46405 | non-targ | GM02LC17485 | fumarate | ARA_LEAF | p-PcUBI | GC | 67 | 224 |
| 84198 | non-targ | GM02LC21368 | citramalate | ARA_LEAF | p-PcUBI | GC | 42 | 177 |
| 84198 | non-targ | GM02LC21368 | fumarate | ARA_LEAF | p-PcUBI | GC | 71 | 316 |
| 84198 | non-targ | GM02LC21368 | malate | ARA_LEAF | p-PcUBI | GC | 102 | 632 |
| 47076 | non-targ | GM02LC5744 | citramalate | ARA_LEAF | p-PcUBI | GC | 47 | 69 |
| 47076 | non-targ | GM02LC5744 | fumarate | ARA_LEAF | p-PcUBI | LC | 49 | 104 |
| 48138 | non-targ | Sll0170 | citramalate | ARA_LEAF | p-PcUBI | GC | 49 | 116 |
| 84265 | plastidic | Sll0228 | fumarate | ARA_LEAF | p-PcUBI | GC | 67 | 78 |
| 84441 | plastidic | Sll0240 | malate | ARA_LEAF | p-PcUBI | GC | 73 | 176 |
| 84441 | plastidic | Sll0240 | succinate | ARA_LEAF | p-PcUBI | GC | 26 | 91 |
| 51198 | non-targ | Sll0816 | fumarate | ARA_LEAF | p-PcUBI | GC | 64 | 228 |
| 127109 | non-targ | Sll0895 | succinate | ARA_LEAF | p-PcUBI | LC | 34 | 83 |
| 54452 | plastidic | Sll1522 | citramalate | ARA_LEAF | p-PcUBI | GC | 43 | 57 |
| 54452 | plastidic | Sll1522 | malate | ARA_LEAF | p-PcUBI | GC | 55 | 206 |
| 85808 | mitoch | Sll1815 | succinate | ARA_LEAF | p-PcUBI | LC | 35 | 414 |
| 55385 | non-targ | Sll1920 | citramalate | ARA_LEAF | p-PcUBI | GC | 42 | 70 |
| 55385 | mitoch | Sll1920 | malate | ARA_LEAF | p-PcUBI | GC | 63 | 120 |
| 136533 | plastidic | Slr0018 | fumarate | ARA_LEAF | p-PcUBI | GC | 167 | 367 |
| 11990 | plastidic | Slr0338 | citramalate | ARA_LEAF | p-PcUBI | GC | 58 | 138 |
| 56153 | plastidic | Slr0597 | fumarate | ARA_LEAF | p-PcUBI | GC | 65 | 115 |
| 87397 | plastidic | Slr0721 | citramalate | ARA_LEAF | p-PcUBI | GC | 55 | 692 |
| 87397 | plastidic | Slr0721 | succinate | ARA_LEAF | p-PcUBI | GC | 63 | 291 |
| 57734 | non-targ | Slr1096 | glycerate | ARA_LEAF | p-PcUBI | GC | 31 | 42 |
| 57734 | non-targ | Slr1096 | succinate | ARA_LEAF | p-PcUBI | LC | 24 | 59 |
| 136869 | non-targ | Slr1170 | succinate | ARA_LEAF | p-PcUBI | LC | 39 | 81 |
| 116460 | mitoch | Slr1250 | fumarate | ARA_LEAF | p-PcUBI | LC | 53 | 59 |
| 58058 | non-targ | Slr1269 | succinate | ARA_LEAF | p-PcUBI | LC | 32 | 75 |
| 58731 | plastidic | Slr1739 | malate | ARA_LEAF | p-PcUBI | GC | 73 | 318 |
| 59165 | plastidic | Slr2023 | fumarate | ARA_LEAF | p-PcUBI | GC | 108 | 384 |
| 59165 | plastidic | Slr2023 | succinate | ARA_LEAF | p-PcUBI | GC | 38 | 152 |
| 136907 | plastidic | Smr0004 | glycerate | ARA_LEAF | p-PcUBI | GC | 29 | 32 |
| 12698 | non-targ | TTC0019 | fumarate | ARA_LEAF | p-PcUBI | GC | 181 | 395 |
| 12698 | non-targ | TTC0019 | succinate | ARA_LEAF | p-PcUBI | LC | 31 | 106 |
| 107556 | non-targ | TTC0849 | citramalate | ARA_LEAF | p-PcUBI | GC | 40 | 352 |
| 61723 | non-targ | TTC1193 | fumarate | ARA_LEAF | p-PcUBI | LC | 72 | 132 |
| 61723 | non-targ | TTC1193 | succinate | ARA_LEAF | p-PcUBI | LC | 36 | 110 |
| 62079 | non-targ | TTC1386 | fumarate | ARA_LEAF | p-PcUBI | GC | 64 | 848 |
| 62079 | non-targ | TTC1386 | malate | ARA_LEAF | p-PcUBI | GC | 241 | 353 |
| 62079 | non-targ | TTC1386 | succinate | ARA_LEAF | p-PcUBI | GC | 31 | 161 |
| 62160 | non-targ | TTC1918 | citramalate | ARA_LEAF | p-PcUBI | GC | 43 | 80 |
| 62160 | non-targ | TTC1918 | glycerate | ARA_LEAF | p-PcUBI | GC | 32 | 61 |
| 62160 | non-targ | TTC1918 | malate | ARA_LEAF | p-PcUBI | GC | 58 | 241 |
| 62244 | non-targ | XM_473199 | fumarate | ARA_LEAF | p-PcUBI | GC | 76 | 257 |
| 62244 | non-targ | XM_473199 | malate | ARA_LEAF | p-PcUBI | GC | 54 | 129 |
| 62244 | non-targ | XM_473199 | succinate | ARA_LEAF | p-PcUBI | LC | 31 | 75 |
| 136943 | plastidic | Yal062w | succinate | ARA_LEAF | p-PcUBI | LC | 35 | 75 |
| 137387 | non-targ | Ybl015w | citramalate | ARA_LEAF | Big35S | GC | 39 | 127 |
| 137387 | non-targ | Ybl015w | pyruvate | ARA_LEAF | Big35S | GC | 44 | 74 |
| 96779 | plastidic | Ybr218c | malate | ARA_LEAF | p-Super | GC | 49 | 99 |
| 63264 | plastidic | Ycr102c | fumarate | ARA_LEAF | p-Super | LC | 49 | 79 |
| 137498 | non-targ | Ydl159w-a | fumarate | ARA_LEAF | Big35S | GC | 18 | 20 |
| 63334 | plastidic | Ydr044w | fumarate | ARA_LEAF | p-PcUBI | GC | 198 | 494 |
| 63334 | plastidic | Ydr044w | succinate | ARA_LEAF | p-PcUBI | GC | 112 | 267 |
| 64177 | non-targ | Ygl237c | malate | ARA_LEAF | Big35S | GC | 54 | 178 |
| 64177 | non-targ | Ygl237c | succinate | ARA_LEAF | Big35S | LC | 33 | 110 |
| 108487 | non-targ | Ygr193c | fumarate | ARA_SEED_2 | p-PcUBI | LC | 50 | 287 |
| 108487 | non-targ | Ygr193c | malate | ARA_SEED_2 | p-PcUBI | LC | 58 | 96 |
| 108487 | non-targ | Ygr193c | succinate | ARA_SEED_2 | p-PcUBI | LC | 129 | 292 |
| 64315 | plastidic | Ygr221c | malate | ARA_LEAF | p-Super | GC | 59 | 80 |
| 101855 | plastidic | Yhl032c | citrate | ARA_SEED_2 | p-USP | LC | 73 | 311 |
| 97620 | plastidic | Yhr131c | malate | ARA_LEAF | p-Super | GC | 53 | 83 |
| 64546 | plastidic | Yhr207c | succinate | ARA_LEAF | p-Super | LC | 59 | 166 |
| 64964 | plastidic | Yjl073w | glycerate | ARA_LEAF | p-Super | GC | 31 | 37 |
| 97635 | non-targ | Yjl219w | oxalate | ARA_SEED_2 | p-PcUBI | GC | 33 | 194 |
| 90409 | plastidic | Yjr148w | citramalate | ARA_LEAF | p-Super | GC | 97 | 244 |
| 66274 | non-targ | Yjr153w | fumarate | ARA_LEAF | p-PcUBI | LC | 50 | 138 |
| 66274 | non-targ | Yjr153w | malate | ARA_LEAF | p-PcUBI | GC | 63 | 166 |
| 90602 | plastidic | YKL038W | malate | ARA_LEAF | p-PcUBI | GC | 67 | 159 |
| 137502 | non-targ | Ykl140w | vitc a/o thra | ARA_SEED_2 | p-PcUBI | GC | 39 | 145 |
| 66695 | plastidic | Ylr065c | fumarate | ARA_LEAF | p-Super | LC | 33 | 45 |
| 137547 | plastidic | Ylr174w | succinate | ARA_LEAF | p-Super | GC | 29 | 48 |
| 129123 | plastidic | Ylr326w | fumarate | ARA_LEAF | p-Super | GC | 71 | 254 |
| 129123 | plastidic | Ylr326w | glycerate | ARA_LEAF | p-Super | GC | 37 | 72 |
| 129123 | plastidic | Ylr326w | vitc a/o thra | ARA_LEAF | p-Super | GC | 30 | 33 |
| 15175 | plastidic | Yml084w | fumarate | ARA_LEAF | p-Super | LC | 30 | 80 |
| 15175 | plastidic | Yml084w | succinate | ARA_LEAF | p-Super | LC | 53 | 165 |
| 137818 | plastidic | Yol073c | citramalate | ARA_LEAF | p-Super | GC | 43 | 59 |
| 137818 | plastidic | Yol073c | malate | ARA_LEAF | p-Super | GC | 62 | 188 |
| 15179 | plastidic | Yol160w | vitc a/o thra | ARA_LEAF | p-Super | GC | 28 | 43 |
| 92668 | non-targ | Ypl028w | malate | ARA_LEAF | Big35S | LC | 41 | 88 |
| 93218 | non-targ | Ypr138c | vitc a/o thra | ARA_SEED_2 | p-USP | GC | 37 | 125 |
| 15187 | non-targ | 47266012_SOYBEAN | fumarate | ARA_LEAF | p-PcUBI | GC | 75 | 378 |
| 15187 | non-targ | 47266012_SOYBEAN | malate | ARA_LEAF | p-PcUBI | GC | 59 | 201 |
| 15187 | non-targ | 47266012_SOYBEAN | succinate | ARA_LEAF | p-PcUBI | GC | 46 | 112 |
| 69 | non-targ | 49747384_SOYBEAN | fumarate | ARA_LEAF | p-PcUBI | GC | 100 | 340 |
| 69 | non-targ | 49747384_SOYBEAN | malate | ARA_LEAF | p-PcUBI | GC | 54 | 202 |
| 69 | non-targ | 49747384_SOYBEAN | succinate | ARA_LEAF | p-PcUBI | LC | 46 | 129 |
| 69 | non-targ | 49747384_SOYBEAN | vitc a/o thra | ARA_LEAF | p-PcUBI | GC | 32 | 64 |
| 15532 | plastidic | 51340801_CANOLA | citramalate | ARA_LEAF | p-PcUBI | GC | 49 | 420 |
| 15532 | plastidic | 51340801_CANOLA | succinate | ARA_LEAF | p-PcUBI | LC | 31 | 210 |
| 16883 | plastidic | 59582753_SOYBEAN | citramalate | ARA_LEAF | p-PcUBI | GC | 70 | 246 |

Column 1 shows the SEQ ID NO, Column 2 shows the expression type (targeted or non-targeted), Column 3 shows the “gene name” (locus), Column 4 shows the metabolite analyzed, Column 5 indicates the A. thaliana source tissue analyzed, Column 6 indicates the used promoter for expression, Column 7 indicates the analytical method. Columns 8 and 9 show the minimum and the maximum increase of the analyzed metabolite (in percent) in comparison to the wild type (ratio_by_WT, given as percent increase).

The term “non-targ” in Table d, Column 2 which shows the expression type means “non-targeted”.

The term “mitoch” in Table d, Column 2 which shows the expression type means “mitochondrial”.

The term “vit c a/o thra” in Table d, Column 2 which shows the expression type means “vitamin c and/or threonic acid”.

When the analyses were repeated independently, all results proved to be significant. [0505.1.7.15] to [0515.1.7.15] for the disclosure of these paragraphs see [0505.1.7.7] to [0515.1.7.7] above

In a further embodiment, the present invention relates in paragraphs [0000.1.7.16.] to [0514.1.7.16.] to a further process for the production of the fine chemical coenzyme Q10, coenzyme Q9, lipoic acid, and/or nicotinic acid as defined below and corresponding embodiments as described herein as follows.

for the disclosure of this paragraph see [0001.1.7.7] above

Cofactors are non-protein chemical compounds which bound to enzymes. They are required for the biological activity of the enzymes and function in association with these. In other words cofactors are required by inactive apoenzymes to convert them into active holoenzymes.

Cofactors can be divided into two broad groups: organic cofactors, such as flavin or heme, and inorganic cofactors: such as the metal ions Mg2+, Cu+, Mn2+or iron-sulfur clusters.

Organic cofactors are small organic molecules (typically a molecular mass less than 1000 Da) that can be either loosely or tightly bound to the enzyme and directly participate in the reaction. Vitamins can serve as precursors to many organic cofactors (e.g. vitamins B1, B2, B6, B12, niacin, folic acid). Many organic cofactors also contain a nucleotide: such as the electron carriers NAD and FAD, or coenzyme A, which carries acyl groups. Most of these cofactors are found in a huge variety of species, and some are universal to all forms of life.

Like enzymes, cofactors are not irreversibly changed during catalysis; they are either unmodifled or regenerated. Each kind of cofactor has a particular chemical function. Cofactors may either be attached by covalent bonds to a particular enzyme or exist freely in solution, but in either case they participate intimately in the chemical reactions catalyzed by the enzyme.

A well known cofactor is Coenzyme Q10 (CoQ 10) or ubiquinone, which is essentially a vitamin or vitamin-like substance. Disagreements on nomenclature notwithstanding, vitamins are defined as organic compounds essential in minute amounts for normal body function acting as coenzymes or precursors to coenzymes. Coenzyme Q10 or CoQ10 belongs to a family of substances called ubiquinones. Ubiquinones, also known as coenzymes Q and mitoquinones, are lipophilic, water-insoluble substances involved in electron transport and energy production in mitochondria. The basic structure of ubiquinones consists of a benzoquinone “head” and a terpinoid “tail.” The “head” structure participates in the redox activity of the electron transport chain. The major difference among the various coenzymes Q is in the number of isoprenoid units (5-carbon structures) in the “tail.” Coenzymes Q contain one to 12 isoprenoid units in the “tail”; 10 isoprenoid units are common in animals. Coenzymes Q occur in the majorganism ity of aerobic organisms, from bacteria to plants and animals. Two numbering systems exist for designation of the number of isoprenoid units in the terpinoid “tail”: coenzyme Qn and coenzyme Q(x). N refers to the number of isoprenoid side chains, and×refers to the number of carbons in the terpinoid “tail” and can be any multiple of five. Thus, coenzyme Q10 refers to a coenzyme Q having 10 isoprenoid units in the “tail.” Since each isoprenoid unit has five carbons, coenzyme Q10 can also be designated coenzyme Q(50). The structures of coenzymes Q are analogous to those of vitamin K2. Coenzyme Q10 is also known as Coenzyme Q(50), CoQ10,

CoQ(50), ubiquinone (50), ubiquino-10 and ubidecarerone.

They are present naturally in foods and sometimes are also synthesized in the body. CoQ10 likewise is found in small amounts in a wide variety of foods and is synthesized in all tissues. The biosynthesis of CoQ10 from the amino acid tyrosine is a multistage process requiring at least eight vitamins and several trace elements. Coenzymes are cofactors upon which the comparatively large and complex enzymes absolutely depend for their function. Coenzyme Q10 is the coenzyme for at least three mitochondrial enzymes (complexes I, II and III) as well as enzymes in other parts of the cell. Mitochondrial enzymes of the oxidative phosphorylation pathway are essential for the production of the high-energy phosphate, adenosine triphosphate (ATP), upon which all cellular functions depend. The electron and proton transfer functions of the quinone ring are of fundamental importance to all life forms; ubiquinone in the mitochondria of animals, plastoquinone in the chloroplast of plants, and menaquinone in bacteria. The term “bioenergetics” has been used to describe the field of biochemistry looking specifically at cellular energy production. In the related field of free radical chemistry, CoQ10 has been studied in its reduced form as a potent antioxidant. The bioenergetics and free radical chemistry of CoQ10 are reviewed in Gian Paolo Littarru's book, Energy and Defense, published in 1994. The precise chemical structure of CoQ10 is 2,3 dimethoxy-5 methyl-6 decaprenyl benzoquinone

Discovered in 1957, CoQ-10 is also called ubiquinone because it belongs to a class of compounds called quinones, and because it's ubiquitous in living organisms, especially in the heart, liver, and kidneys. It plays a crucial role in producing energy in cells. And it acts as a powerful antioxidant, meaning that it helps neutralize cell-damaging molecules called free radicals. Manufactured by all cells in the body, CoQ-10 is also found in small amounts in foods, notably meat and fish. By the mid-1970′s, the industrial technology to produce pure CoQ10 in quantities sufficient for larger clinical trials was established. Principally CoQ10 can be isolated from microorganisms or plants or algae; in particular mitochondria are a common source for CoQ10. Alternatively, they are obtained advantageously from animals or fish.

Since the actions of supplemental CoQ10 have yet to be clarified, the mechanism of these actions is a matter of speculation. However, much is known about the biochemistry of CoQ10. CoQ10 is an essential cofactor in the mitochondrial electron transport chain, where it accepts electrons from complex I and II, an activity that is vital for the production of ATP. CoQ10 has antioxidant activity in mitochondria and cellular membranes, protecting against peroxidation of lipid membranes. It also inhibits the oxidation of LDL-cholesterol. LDL-cholesterol oxidation is believed to play a significant role in the pathogenesis of atherosclerosis. CoQ10 is biosynthesized in the body and shares a common synthetic pathway with cholesterol.

CoQ10 levels decrease with aging in humans. Why this occurs is not known but may be due to decreased synthesis and/or increased lipid peroxidation which occurs with aging. Significantly decreased levels of CoQ10 have been noted in a wide variety of diseases in both animal and human studies. CoQ10 deficiency may be caused by insufficient dietary CoQ10, impairment in CoQ10 biosynthesis, excessive utilization of CoQ10 by the body, or any combination of the three. Decreased dietary intake is presumed in chronic malnutrition and cachexia.

The relative contribution of CoQ10 biosynthesis versus dietary CoQ10 is under investigation. Karl Folkers takes the position that the dominant source of CoQ10 in man is biosynthesis. This complex, 17 step process, requiring at least seven vitamins (vitamin B2- riboflavin, vitamin B3- niacinamide, vitamin B6, folic acid, vitamin B12, vitamin C, and pantothenic acid) and several trace elements, is, by its nature, highly vulnerable. Karl Folkers argues that suboptimal nutrient intake in man is almost universal and that there is subsequent secondary impairment in CoQ10 biosynthesis. This would mean that average or “normal” levels of CoQ10 are really suboptimal and the very low levels observed in advanced disease states represent only the tip of a deficiency “ice berg”.

Supplemental CoQ10 may have cardioprotective, cytoprotective and neuroprotective activities. There are claims that it has positive effects in cancer, muscular dystrophy and immune dysfunction. Similarly, it may inhibit obesity or enhance athletic performance. HMG-CoA reductase inhibitors used to treat elevated blood cholesterol levels by blocking cholesterol biosynthesis also block CoQ10 biosynthesis. The resulting lowering of blood CoQ10 level is due to the partially shared biosynthetic pathway of CoQ10 and cholesterol. In patients with heart failure this is more than a laboratory observation. It has a significant harmful effect which can be negated by oral CoQ10 supplementation.

Increased body consumption of CoQ10 is the presumed cause of low blood CoQ10 levels seen in excessive exertion, hypermetabolism, and acute shock states. It is likely that all three mechanisms (insufficient dietary CoQ10, impaired CoQ10 biosynthesis, and excessive utilization of CoQ10) are operable to varying degrees in most cases of observed CoQ10 deficiency.

In nature, Coenzymes QO to Q9 are found as well. E.g. Coenzyme Q9 is a derivative of CoQ10 found e.g. in the chloroplast of plants. Coenzyme Q9 has a shorter aliphatic group bound to the ring structure. Due to the high structural homology of Coenzymes QO to Q9 are expected to provide the same or very similar activities as CoQ10 in cells or organisms.

However, Matsura et al., Biochim Biophys Acta, 1992, 1123(3) pp. 309-15 concluded from their study that CoQ9 constantly acts as a potential antioxidant in hepatocytes whereas CoQ10 manly exhibit its antioxidant activity in cells containing CoQ10 as the predominate CoQ homolog. Coenzyme Q10 is actual a very common ingredient in different types of cosmetics, due to its protective role against radicals and its predicted function in skin tautening.

Lipoic acid (lipoate) is a sulfur-containing cofactor involved in several multienzyme complexes such as pyruvate dehydrogenase, a-ketoglutarate dehydrogenase, branchedchain keto acid dehydrogenase, and glycine decarboxylase complex. Lipoic acid is also knon as (R)-5-(1,2-dithiolan-3-yl)pentanoic acid, alpha lipoic acid, 6,8-dithiooctanoic acid with a CAS-No: 1200-22-2.

Lipoic acid contains on chiral carbon atom. The R-(+)-enantiomere (RLA) is disovered in nature and is an essential cofactor for mitochondrial enzyme complexes.

One decade ago, several studies highlighted the potential of free lipoic acid and dihydrolipoic acid as powerful metabolic antioxidants that are able to scavenge the reactive oxygen species, to recycle other antioxidants (vitamin C, glutathione, and vitamin E), and even to intervene in redox regulation of gene transcription. Consequently, lipoic acid saw an upshift in use as a therapeutic agent in pathologies associated with oxidative stress (Gueguen et al., JOURNAL OF BIOLOGICAL CHEMISTRY (2000), Vol. 275, No. 7, pp. 5016-5025).

In the cell, very little lipoate exists as free acid almost all is tethered (via a peptide bond involving the lipoic acid carboxyl function) to the side chain amino group of a conserved lysine resiude in some enzyme complex dependent of lipoic acid as cofactor. (Booker, S., Chemistry & Biology, 2004, 10-12)

Nicotinic acid (also known as vitamin B3, niacin, nicotinate, vitamin PP, pyridine-3-carboxylic acid and vitamin PP and the CAS NO 59-67-6) is an organic cofactor and one of the forty to eighty essential human nutrients. This colorless, water-soluble solid is a derivative of pyridine, with a carboxyl group (COOH) at the 3-position. Nicotinic acid and its amide derivative, nicotinamide (niacinamide) are biosynthetically linked the coenzymes nicotinamide adenine dinucleotide (NAD) and nicotinamide adenine dinucleotide phosphate (NADP).

The late stage of severe niacin deficiency is known as pellagra. Niacin deficiency or pellagra may result from inadequate dietary intake of niacin and/or tryptophan. Other nutrient deficiencies may also contribute to the development of niacin deficiency. For instance, patients with

Hartnup's disease, a hereditary disorder resulting in defective tryptophan absorption, have developed pellagra.

Nicotinic acid is used as food supplement and for food fortification. Niacin is found in variety of foods, including liver, chicken, beef, fish, cereal, peanuts and legumes, and is also synthesized from tryptophan, which is found in meat, dairy and eggs.

Thus, cofactors, in particular coenzyme Q10, coenzyme Q9, lipoic acid, and/or nicotinic acid can be used in a lot of different applications, for example in cosmetics, pharmaceuticals and in feed and food.

Therefore improving the productivity of said cofactors and improving the quality of cosmetics, pharmaceuticals, foodstuffs and animal feeds, in particular of nutrition supplements, is an imporganism tant task of the different industries.

The content of coenzyme Q10 and coenzyme Q9 in a cell was also increased by increasing or decreasing certain activities as disclosed in WO2006/069610, WO2007/087815 or WO2008/034648.

To ensure a high productivity of said cofactors, in particular coenzyme Q10, coenzyme Q9, lipoic acid, and/or nicotinic acid in plants or microorganism, it is necessary to manipulate the natural biosynthesis of said cofactors in said organisms.

Accordingly, there is still a great demand for new and more suitable genes which encode enzymes or other regulators which participate in the biosynthesis of said cofactors, in particular coenzyme Q10, coenzyme Q9, lipoic acid, and/or nicotinic acid and make it possible to produce certain said cofactors specifically on an industrial scale without unwanted byproducts forming. In the selection of genes for biosynthesis two characteristics above all are particularly important. On the one hand, there is as ever a need for improved processes for obtaining the highest possible contents of said cofactors on the other hand as less as possible byproducts should be produced in the production process.

It is an object of the present invention to develop an inexpensive process for the synthesis of coenzyme Q10, coenzyme Q9, lipoic acid, and/or nicotinic acid.

It is a further object of the present invention to develop an inexpensive process for the synthesis of cofactors, in particular coenzyme Q10, coenzyme Q9, lipoic acid, and/or nicotinic acid and to assure that the cofactors, in particular coenzyme Q10, coenzyme Q9, lipoic acid, and/or nicotinic acid are more accessible and facilely to isolate and recover in an industrial scale from the producing organism, preferably from a plant.

for the disclosure of this paragraph see [0013.1.7.7] above

Accordingly, in a first embodiment, the invention relates to a process for the production of at least one fine chemical selected from the group of cofactors, in particular from the group consisting of: coenzyme Q10, coenzyme Q9, lipoic acid, and/or nicotinic acid, or, in other words, of the “fine chemical” or “fine chemical of the invention”.

The terms “fine chemical of the invention”, “fine chemical” or “the fine chemical” are used herein equally and relate in context of the paragraphs or sections [0014.1.16.16] to [0514.1.7.16] essentially to the metabolite or the metabolites indicated in column 7, application no. 16 of Tables I, II and IV in the respective line.

Further, the term “in context of any of the paragraphs [0014.1.16.16] to [0514.1.7.16]” as used herein means that for any of said paragraphs [0014.1.16.16] to [0514.1.7.16] the term “the fine chemical” is understood to follow the definition of paragraphs or sections [0014.1.16.16] and [0015.1.16.16], independently whether it refers to any other paragraph or not and whether the reference recites the term “fine chemical” in an other context.

Thus, in cases where one or more paragraphs or sections are incorporated by reference into any of the present paragraphs [0014.1.16.16] to [0514.1.7.16], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to the definition of paragraph [0014.1.16.16] and [0015.1.16.16].

Accordingly, in one embodiment, the term “fine chemical of the invention”, “fine chemical” or “the fine chemical” as used herein (alone or in combination, like in FCRP) means “coenzyme Q10” in context of the nucleic acid or polypeptide sequences listed in the respective same line of any one of Tables I to IV of application no. 16 and indicating in column 7 the metabolite “coenzyme Q10”.

In one embodiment, the term coenzyme Q10 or the term “fine chemical” mean in context of the paragraphs or sections [0014.1.16.16] to [0514.1.7.16] at least one chemical compound with an activity of the above mentioned coenzyme Q10, respectively.

Accordingly, in one embodiment, the term “fine chemical of the invention”, “fine chemical” or “the fine chemical” as used herein (alone or in combination, like in FCRP) means “coenzyme Q9” in context of the nucleic acid or polypeptide sequences listed in the respective same line of any one of Tables I to IV of application no. 16 and indicating in column 7 the metabolite “coenzyme Q9”.

In one embodiment, the term coenzyme Q9 or the term “fine chemical” mean in context of the paragraphs or sections [0014.1.16.16] to [0514.1.7.16] at least one chemical compound with an activity of the above mentioned coenzyme Q9, respectively.

Accordingly, in one embodiment, the term “fine chemical of the invention”, “fine chemical” or “the fine chemical” as used herein (alone or in combination, like in FCRP) means “lipoic acid” in context of the nucleic acid or polypeptide sequences listed in the respective same line of any one of Tables Ito IV of application no. 16 and indicating in column 7 the metabolite “lipoic acid”.

In one embodiment, the term lipoic acid or the term “fine chemical” mean in context of the paragraphs or sections [0014.1.16.16] to [0514.1.7.16] at least one chemical compound with an activity of the above mentioned lipoic acid, respectively.

Accordingly, in one embodiment, the term “fine chemical of the invention”, “fine chemical” or “the fine chemical” as used herein (alone or in combination, like in FCRP) means “nicotinic acid” in context of the nucleic acid or polypeptide sequences listed in the respective same line of any one of Tables I to IV of application no. 16 and indicating in column 7 the metabolite “nicotinic acid”.

In one embodiment, the term nicotinic acid or the term “fine chemical” mean in context of the paragraphs or sections [0014.1.16.16] to [0514.1.7.16] at least one chemical compound with an activity of the above mentioned nicotinic acid, respectively.

Accordingly, in one embodiment, the terms “fine chemical of the invention”, “fine chemical” or “the fine chemical” mean in context of any of the paragraphs [0014.1.16.16] to [0514.1.7.16] coenzyme Q10, in any one of the three redox states of Coenzyme Q10: fully oxidized (ubiquinone), semiquinone (ubisemiquinone), and fully reduced (ubiquinol), their salts, ester or ether in free form or bound form. In a preferred embodiment, the term “the fine chemical” means coenzyme Q10 in any one of the three redox states of Coenzyme Q10: fully oxidized (ubiquinone), semiquinone (ubisemiquinone), and fully reduced (ubiquinol), or their salts, ester or ether, in free form or bound form, preferably bound to peptide(s) or protein(s). In a preferred embodiment “coenzyme Q10” means coenzyme Q10 in free form.

Accordingly, in one embodiment, the terms “fine chemical of the invention”, “fine chemical” or “the fine chemical” mean in context of any of the paragraphs [0014.1.16.16] to [0514.1.7.16] coenzyme Q9, in any one of the three redox states of Coenzyme Q9: fully oxidized, semiquinone, and fully reduced, their salts, ester or ether in free form or bound form. In a preferred embodiment, the term “the fine chemical” means coenzyme Q9 in any one of the three redox states of Coenzyme Q9: fully oxidized, semiquinone, and fully reduced, or their salts, ester or ether, in free form or bound form, preferably bound to peptide(s) or protein(s).

In a preferred embodiment “coenzyme Q9” means coenzyme Q9 in free form.

Accordingly, in one embodiment, the terms “fine chemical of the invention”, “fine chemical” or “the fine chemical” mean in context of any of the paragraphs [0014.1.16.16] to [0514.1.7.16] lipoic acid, its salts, ester or ether in free form or bound form. In a preferred embodiment, the term “the fine chemical” means lipoic acid or its salts, ester or ether, in free form or bound form, preferably bound to peptide(s) or protein(s).

In a preferred embodiment “lipoic acid” means lipoic acid in free form.

The state of (de-)protonsation is not considered here (lipoic acid treated as lipoate and visa versa).

Accordingly, in one embodiment, the terms “fine chemical of the invention”, “fine chemical” or “the fine chemical” mean in context of any of the paragraphs [0014.1.16.16] to [0514.1.7.16] nicotinic acid, its salts, ester or ether in free form or bound form. In a preferred embodiment, the term “the fine chemical” means nicotinic acid or its salts, ester or ether, in free form or bound form, preferably bound to peptide(s) or protein(s).

In a preferred embodiment “nicotinic acid” means nicotinic acid in free form.

The state of (de-)protonsation is not considered here (nicotinic acid treated as nicotinate and visa versa). The terms nicotinic acid, niacin, nicotinamide, and vitamin B3 are used interchangeably to refer to any member of this family of compounds, since they have the same biochemical activity.

Further, the term “in context of any of the paragraphs [0014.1.16.16] to [0514.1.7.16]” as used herein means that for any of said paragraphs [0014.1.16.16] to [0514.1.7.16] the term “the fine chemical” is understood to follow the definition of section [0014.1.16.16] or section [0015.1.16.16], independently whether it refers to any other paragraph or not and whether the reference recites the term “fine chemical” in an other context.

Thus, in cases where one or more paragraphs or sections are incorporated by reference into any of the present paragraphs [0014.1.16.16] to [0514.1.7.16], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to this definition of this paragraph [0015.1.16.16].

Further, the term “fine chemicals” as used herein relates to compositions comprising said fine chemical(s), i.e. comprising coenzyme Q10, coenzyme Q9, lipoic acid, and/or nicotinic acid, respectively.

Accordingly, the present invention relates to a process for the production of coenzyme Q10, which comprises
(a) increasing or generating one or more activities selected from the group consisting of amino acid acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the pro-duction of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of At1 g47380-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of phenylalanine ammonia-lyase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of MADS box protein transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating one or more activities selected from the group consisting of F-box protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating one or more activities selected from the group consisting of CCAAT-binding transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating one or more activities selected from the group consisting of CCAAT-binding transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of glycosyl transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of auxin response factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating one or more activities selected from the group consisting of sterol desaturase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q10, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating one or more activities selected from the group consisting of cy- clin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the pro- duction of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating one or more activities selected from the group consisting of disease resistance protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of zinc finger protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of CTP synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of recombinase A in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating one or more activities selected from the group consisting of enoyl-CoA hydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating one or more activities selected from the group consisting of enoyl-CoA hydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of isopropylmalate isomerase large subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of AX653549-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating one or more activities selected from the group consisting of AY087308-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q10, which comprises
(a) increasing or generating one or more activities selected from the group consisting of threonine synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q10, which comprises
(a) increasing or generating one or more activities selected from the group consisting of acetolactate synthase III large subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating one or more activities selected from the group consisting of serine protease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of beta-galactosidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating one or more activities selected from the group consisting of ATP-binding component of a transport system in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating one or more activities selected from the group consisting of ATP-binding component of a transport system in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating one or more activities selected from the group consisting of zinc transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating one or more activities selected from the group consisting of zinc transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating one or more activities selected from the group consisting of methylglyoxal synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating one or more activities selected from the group consisting of methylglyoxal synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of b1003-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of b1097-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating one or more activities selected from the group consisting of b1522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating one or more activities selected from the group consisting of b1522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating one or more activities selected from the group consisting of acid shock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating one or more activities selected from the group consisting of acid shock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of serine/threonine-protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of b2032-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating one or more activities selected from the group consisting of colanic acid biosynthesis protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating one or more activities selected from the group consisting of NADH dehydrogenase I chain I in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of nicotinic acid, which comprises
(a) increasing or generating one or more activities selected from the group consisting of ATP synthase subunit E in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of nicotinic acid or a composition comprising nicotinic acid in said non-human organism or in the culture medium surrounding said non-human organism.

Accordingly, the present invention relates to a process for the production of nicotinic acid, which comprises
(a) increasing or generating one or more activities selected from the group consisting of L-aspartate oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of nicotinic acid or a composition comprising nicotinic acid in said non-human organism or in the culture medium surrounding said non-human organism.

Accordingly, the present invention relates to a process for the production of coenzyme Q9, which comprises
(a) increasing or generating one or more activities selected from the group consisting of b2739-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of 2-C-methyl-D-erythritol 4-phosphate cytidylyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of lipoic acid, which comprises
(a) increasing or generating one or more activities selected from the group consisting of phosphoadenosine phosphosulfate reductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lipoic acid or a composition comprising lipoic acid in said non-human organism or in the culture medium surrounding said non-human organism.

Accordingly, the present invention relates to a process for the production of coenzyme Q9, which comprises
(a) increasing or generating one or more activities selected from the group consisting of b2849-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating one or more activities selected from the group consisting of Ribose import ATP-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating one or more activities selected from the group consisting of b3817-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of glycerol dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating one or more activities selected from the group consisting of N-acetyl-gamma-glutamylphosphate reductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating one or more activities selected from the group consisting of b4029-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of gluconate transport system permease 3 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q10, which comprises
(a) increasing or generating one or more activities selected from the group consisting of flavodoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q10, which comprises
(a) increasing or generating one or more activities selected from the group consisting of uroporphyrin-III C-methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q10, which comprises
(a) increasing or generating one or more activities selected from the group consisting of sll1761-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating one or more activities selected from the group consisting of sll1761-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of cation-transporting ATPase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating one or more activities selected from the group consisting of malate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating one or more activities selected from the group consisting of malate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of permease protein of ABC transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of slr1107-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of fatty acid desaturase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of photosystem II protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating one or more activities selected from the group consisting of glutamine amidotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q10, which comprises
(a) increasing or generating one or more activities selected from the group consisting of glycerol-3-phosphate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating one or more activities selected from the group consisting of glycerol-3-phosphate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of Sec-independent protein translocase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating one or more activities selected from the group consisting of amino acid ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating one or more activities selected from the group consisting of multiple antibiotic resistance protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of TTC1386-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of cell division control protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating one or more activities selected from the group consisting of ycl027w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of halotolerance protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of nicotinic acid, which comprises
(a) increasing or generating one or more activities selected from the group consisting of lipase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of nicotinic acid or a composition comprising nicotinic acid in said non-human organism or in the culture medium surrounding said non-human organism.

Accordingly, the present invention relates to a process for the production of coenzyme Q10, which comprises
(a) increasing or generating one or more activities selected from the group consisting of heat shock protein Hsp40 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating one or more activities selected from the group consisting of heat shock protein Hsp40 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating one or more activities selected from the group consisting of histone acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating one or more activities selected from the group consisting of mitogen-activated protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q10, which comprises
(a) increasing or generating one or more activities selected from the group consisting of phosphopantothenoylcysteine decarboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

An embodiment of the present invention relates to a process for the production of coenzyme Q10, which comprises
(a1) increasing or generating the activity of a polypeptide comprising a polypeptide as depicted in a respective line in column 5 or 8 of Table II, application no. 16, or a homolog or a fragment thereof, a consensus sequence or at least one polypeptide motif as depicted in the respective line in column 8 of Table IV, application no. 16, whereby the respective line disclose in column 7 the fine chemical coenzyme Q10; or
(a2) increasing or generating the activity of an expression product of one or more nucleic acid molecule(s) comprising a polynucleotide as depicted in the respective line in column 5 or 8 of Table I, application no. 16, preferably the coding region thereof, or a homolog or a fragment thereof, whereby the respective line disclose in column 7 the fine chemical coenzyme Q10; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of coenzyme Q10 or a composition comprising coenzyme Q10 in said non-human organism or in the culture medium surrounding said non-human organism.

An embodiment of the present invention relates to a process for the production of coenzyme Q9, which comprises
(a1) increasing or generating the activity of a polypeptide comprising a polypeptide as depicted in a respective line in column 5 or 8 of Table II, application no. 16, or a homolog or a fragment thereof, a consensus sequence or at least one polypeptide motif as depicted in the respective line in column 8 of Table IV, application no. 16, whereby the respective line disclose in column 7 the fine chemical coenzyme Q9; or
(a2) increasing or generating the activity of an expression product of one or more nucleic acid molecule(s) comprising a polynucleotide as depicted in the respective line in column 5 or 8 of Table I, application no. 16, preferably the coding region thereof, or a homolog or a fragment thereof, whereby the respective line disclose in column 7 the fine chemical coenzyme Q9; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of coenzyme Q9 or a composition comprising coenzyme Q9 in said non-human organism or in the culture medium surrounding said non-human organism.

An embodiment of the present invention relates to a process for the production of lipoic acid, which comprises
(a1) increasing or generating the activity of a polypeptide comprising a polypeptide as depicted in a respective line in column 5 or 8 of Table II, application no. 16, or a homolog or a fragment thereof, a consensus sequence or at least one polypeptide motif as depicted in the respective line in column 8 of Table IV, application no. 16, whereby the respective line disclose in column 7 the fine chemical lipoic acid; or
(a2) increasing or generating the activity of an expression product of one or more nucleic acid molecule(s) comprising a polynucleotide as depicted in the respective line in column 5 or 8 of Table I, application no. 16, preferably the coding region thereof, or a homolog or a fragment thereof, whereby the respective line disclose in column 7 the fine chemical lipoic acid; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of lipoic acid or a composition comprising lipoic acid in said non-human organism or in the culture medium surrounding said non-human organism.

An embodiment of the present invention relates to a process for the production of nicotinic acid, which comprises
(a1) increasing or generating the activity of a polypeptide comprising a polypeptide as depicted in a respective line in column 5 or 8 of Table II, application no. 16, or a homolog or a fragment thereof, a consensus sequence or at least one polypeptide motif as depicted in the respective line in column 8 of Table IV, application no. 16, whereby the respective line disclose in column 7 the fine chemical nicotinic acid; or
(a2) increasing or generating the activity of an expression product of one or more nucleic acid molecule(s) comprising a polynucleotide as depicted in the respective line in column 5 or 8 of Table I, application no. 16, preferably the coding region thereof, or a homolog or a fragment thereof, whereby the respective line disclose in column 7 the fine chemical nicotinic acid; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of nicotinic acid or a composition comprising nicotinic acid in said non-human organism or in the culture medium surrounding said non-human organism.

Accordingly, the increase or generation of one or more said activities is for example conferred by one or more expression products of said nucleic acid molecule, e.g. proteins.

Accordingly, in the present invention described above, the increase or generation of one or more said activities is for example conferred by one or more protein(s) each comprising a polypeptide selected from the group as depicted in Table II, application no. 16, column 5 and 8, or a homolog or a fragment thereof.

The process of the invention comprises in one embodiment the following steps:
(i) increasing or generating of the expression of; and/or
(ii) increasing or generating the expression of an expression product of; and/or
(iii) increasing or generating one or more activities of an expression product encoded by;
at least one nucleic acid molecule (in the following “Fine Chemical Related Protein (FCRP)”-encoding gene or “FCRP”-gene) comprising a nucleic acid molecule selected from the group consisting of:
(a) a nucleic acid molecule encoding the polypeptide shown in column 5 or 8 of Table II, preferably Table II B, application no. 16, or a homolog or a fragment thereof;
(b) a nucleic acid molecule shown in column 5 or 8 of Table I, preferably Table I B, application no. 16, or a homolog or a fragment thereof (preferably the coding region thereof);
(c) a nucleic acid molecule, which, as a result of the degeneracy of the genetic code, can be derived from a polypeptide sequence depicted in column 5 or 8 of Table II, preferably Table II B, application no. 16;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 16, or the coding region thereof;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d),
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 16;
(i) a nucleic acid molecule encoding a polypeptide having the activity represented by a respective protein as depicted in column 5 of Table II, application no. 16;
(j) a nucleic acid molecule which comprises a polynucleotide, which is obtained by amplifying a cDNA library or a genomic library using the primers as depicted in column 8 of Table III, application no. 16; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment thereof said nucleic acid molecule encodes a polypeptide which has the activity of the polypeptide represented by a protein comprising a polypeptide as depicted in the corresponding hit in column 5 of Table II, application no. 16.

In another preferred embodiment thereof said nucleic acid molecule confers a generation or an increase of the respective fine chemical in a non-human organism or a part thereof as compared to a corresponding wild-type non-human organism or part thereof.

In another preferred embodiment thereof said nucleic acid molecule encodes a polypeptide which has the activity of the polypeptide represented by a protein as depicted in the corresponding hit in column 5 of Table II, application no. 16, and confers a generation or an increase of the respective fine chemical in a non-human organism or a part thereof as compared to a corresponding wild-type non-human organism or part thereof.

Accordingly, the genes of the present invention or used in accordance with the present invention, which respectively encode a protein having an activity of 2-C-methyl-D-erythritol 4-phosphate cytidylyltransferase, acetolactate synthase III large subunit, acetyltransferase, acid shock protein, amino acid ABC transporter permease protein, amino acid acetyltransferase, At1g47380-protein, ATP synthase subunit E, ATP-binding component of a transport system, auxin response factor, AX653549-protein, AY087308-protein, b1003-protein, b1097-protein, b1522-protein, b2032-protein, b2739-protein, b2849-protein, b3817-protein, b4029-protein, beta-galactosidase, calcium-dependent protein kinase, cation-transporting ATPase, CCAAT-binding transcription factor, cell division control protein, colanic acid biosynthesis protein, CTP synthase, cyclin D , disease resistance protein, enoyl-CoA hydratase, fatty acid desaturase, F-box protein, flavodoxin, gluconate transport system permease 3, glutamine amidotransferase, glutaredoxin, glycerol dehydrogenase, glycerol-3-phosphate dehydrogenase, glycosyl transferase, halotolerance protein, heat shock protein Hsp40, heat shock transcription factor, histone acetyltransferase, hydrolase, isopropylmalate isomerase large subunit, L-aspartate oxidase, lipase, MADS box protein transcription factor, malate dehydrogenase, methylglyoxal synthase, methyltransferase, mitogen-activated protein kinase, monothiol glutaredoxin, multiple antibiotic resistance protein, N-acetyl-gamma-glutamylphosphate reductase, NADH dehydrogenase I chain I, oxidoreductase, permease protein of ABC transporter, phenylalanine ammonia-lyase, phosphoadenosine phosphosulfate reductase, phosphopantothenoylcysteine decarboxylase, photosystem II protein, protein kinase, protein phosphatase, pyruvate kinase, recombinase A, Ribose import ATP-binding protein, Sec-independent protein translocase subunit, serine protease, serine/threonine-protein phosphatase, sll1761-protein, slr1107-protein, sterol desaturase, threonine synthase, transcription factor, transcriptional regulator, TTC1386-protein, uroporphyrin-111 C-methyltransferase, ycl027w-protein, zinc finger protein, or zinc transporter, which respectively encode a protein comprising a polypeptide encoded by a nucleic acid sequence as shown in Table I, application no. 16, column 5 or 8, (preferably the coding region thereof), or a homolog or a fragment thereof, which respectively encode a protein comprising a polypeptide as depicted in Table II, application no. 16, column 5 or 8, or a homolg or a fragment thereof, and/or which respectively can be amplified with the primer set shown in Table III, application no. 16, column 8, are also referred to as “FCRP genes”.

Proteins or polypeptides encoded by “FCRP-genes” are referred to as “Fine Chemical Related Proteins” or “FCRP”. For the purposes of the description of the present invention, the respective protein having an activity of 2-C-methyl-D-erythritol 4-phosphate cytidylyltransferase, acetolactate synthase III large subunit, acetyltransferase, acid shock protein, amino acid ABC transporter permease protein, amino acid acetyltransferase, At1g47380-protein, ATP synthase subunit E, ATP-binding component of a transport system, auxin response factor, AX653549-protein, AY087308-protein, b1003-protein, b1097-protein, b1522-protein, b2032-protein, b2739-protein, b2849-protein, b3817-protein, b4029-protein, beta-galactosidase, calcium-dependent protein kinase, cation-transporting ATPase, CCAAT-binding transcription factor, cell division control protein, colanic acid biosynthesis protein, CTP synthase, cyclin D , disease resistance protein, enoyl-CoA hydratase, fatty acid desaturase, F-box protein, flavodoxin, gluconate transport system permease 3, glutamine amidotransferase, glutaredoxin, glycerol dehydrogenase, glycerol-3-phosphate dehydrogenase, glycosyl transferase, halotolerance protein, heat shock protein Hsp40, heat shock transcription factor, histone acetyltransferase, hydrolase, isopropylmalate isomerase large subunit, L-aspartate oxidase, lipase, MADS box protein transcription factor, malate dehydrogenase, methylglyoxal synthase, methyltransferase, mitogen-activated protein kinase, monothiol glutaredoxin, multiple antibiotic resistance protein, N-acetyl-gamma-glutamylphosphate reductase, NADH dehydrogenase I chain I, oxidoreductase, permease protein of ABC transporter, phenylalanine ammonia-lyase, phosphoadenosine phosphosulfate reductase, phosphopantothenoylcysteine decarboxylase, photosystem II protein, protein kinase, protein phosphatase, pyruvate kinase, recombinase A, Ribose import ATP-binding protein, Sec-independent protein translocase subunit, serine protease, serine/threonineprotein phosphatase, sll1761-protein, slr1107-protein, sterol desaturase, threonine synthase, transcription factor, transcriptional regulator, TTC1386-protein, uroporphyrin-III C-methyltransferase, ycl027w-protein, zinc finger protein, or zinc transporter, the respective protein comprising a polypeptide encoded by one or more respective nucleic acid sequences as shown in Table I , application no. 16, column 5 or 8, (preferably the coding region thereof), or a homolog or fragment thereof, the respective protein comprising a respective polypeptide as depicted in Table II, application no. 16, column 5 or 8, or a homolog or fragment thereof, the respective protein comprising a sequence corresponding to the consensus sequence as shown in Table IV, application no. 16, column 8, and/or the respective protein comprising at least one polypeptide motif as shown in Table IV, application no. 16, column 8 is also referred to as Fine Chemical Related Protein” or “FCRP”.

Thus, in one embodiment, the present invention provides a process of the production of coenzyme Q10, coenzyme Q9, lipoic acid, and/or nicotinic acid, by increasing or generating one or more activities, especially selected from the group consisting of 2-C-methyl-D-erythritol 4-phosphate cytidylyltransferase, acetolactate synthase III large subunit, acetyltransferase, acid shock protein, amino acid ABC transporter permease protein, amino acid acetyltransferase, At1g47380-protein, ATP synthase subunit E, ATP-binding component of a transport system, auxin response factor, AX653549-protein, AY087308-protein, b1003-protein, b1097-protein, b1522-protein, b2032-protein, b2739-protein, b2849-protein, b3817-protein, b4029-protein, beta-galactosidase, calcium-dependent protein kinase, cation-transporting ATPase, CCAAT-binding transcription factor, cell division control protein, colanic acid biosynthesis protein, CTP synthase, cyclin D , disease resistance protein, enoyl-CoA hydratase, fatty acid desaturase, F-box protein, flavodoxin, gluconate transport system permease 3, glutamine amidotransferase, glutaredoxin, glycerol dehydrogenase, glycerol-3-phosphate dehydrogenase, glycosyl transferase, halotolerance protein, heat shock protein Hsp40, heat shock transcription factor, histone acetyltransferase, hydrolase, isopropylmalate isomerase large subunit, L-aspartate oxidase, lipase, MADS box protein transcription factor, malate dehydrogenase, methylglyoxal synthase, methyltransferase, mitogen-activated protein kinase, monothiol glutaredoxin, multiple antibiotic resistance protein, N-acetyl-gamma-glutamylphosphate reductase, NADH dehydrogenase I chain I, oxidoreductase, permease protein of ABC transporter, phenylalanine ammonia-lyase, phosphoadenosine phosphosulfate reductase, phosphopantothenoylcysteine decarboxylase, photosystem II protein, protein kinase, protein phosphatase, pyruvate kinase, recombinase A, Ribose import ATP-binding protein, Sec-independent protein translocase subunit, serine protease, serine/threonine-protein phosphatase, sll1761-protein, slr1107-protein, sterol desaturase, threonine synthase, transcription factor, transcriptional regulator, TTC1386-protein, uroporphyrin-111 C-methyltransferase, ycl027w-protein, zinc finger protein, and zinc transporter, which is conferred by one or more FCRPs or the gene product of one or more FCRP-genes, for example by the gene product of a nucleic acid sequences comprising a polynucleotide selected from the group as shown in Table I, application no. 16, column 5 or 8, (preferably by the coding region thereof), or a homolog or a fragment thereof, e.g. or by one or more proteins each comprising a polypeptide encoded by one or more nucleic acid sequences selected from the group as shown in Table I, application no. 16, column 5 or 8, (preferably by the coding region thereof), or a homolog or a fragment thereof, or by one or more protein(s) each comprising a polypeptide selected from the group as depicted in Table II, application no. 16, column 5 and 8, or a homolog thereof, or a protein comprising a sequence corresponding to the consensus sequence or comprising at least one polypeptide motif as shown in Table IV, application no. 16, column 8.

for the disclosure of this paragraph see [0025.1.7.7] above

In an embodiment, the process comprises increasing or generating the activity of one or more polypeptides having said activity, e.g. by generating or increasing the amount and/or specific activity in the cell or a compartment of a cell of one of more FCRP, especially selected from the group consisting of 2-C-methyl-D-erythritol 4-phosphate cytidylyltransferase, acetolactate synthase III large subunit, acetyltransferase, acid shock protein, amino acid ABC transporter permease protein, amino acid acetyltransferase, At1g47380-protein, ATP synthase subunit E, ATP-binding component of a transport system, auxin response factor, AX653549-protein, AY087308-protein, b1003-protein, b1097-protein, b1522-protein, b2032-protein, b2739-protein, b2849-protein, b3817-protein, b4029-protein, beta-galactosidase, calcium-dependent protein kinase, cation-transporting ATPase, CCAAT-binding transcription factor, cell division control protein, colanic acid biosynthesis protein, CTP synthase, cyclin D , disease resistance protein, enoyl-CoA hydratase, fatty acid desaturase, F-box protein, flavodoxin, gluconate transport system permease 3, glutamine amidotransferase, glutaredoxin, glycerol dehydrogenase, glycerol-3-phosphate dehydrogenase, glycosyl transferase, halotolerance protein, heat shock protein Hsp40, heat shock transcription factor, histone acetyltransferase, hydrolase, isopropylmalate isomerase large subunit, L-inaspartate oxidase, lipase, MADS box protein transcription factor, malate dehydrogenase, methylglyoxal synthase, methyltransferase, mitogen-activated protein kinase, monothiol glutaredoxin, multiple antibiotic resistance protein, N-acetyl-gamma-glutamylphosphate reductase, NADH dehydrogenase I chain I, oxidoreductase, permease protein of ABC transporter, phenylalanine ammonia-lyase, phosphoadenosine phosphosulfate reductase, phosphopantothenoylcysteine decarboxylase, photosystem 11 -protein, protein kinase, protein phosphatase, pyruvate kinase, recombinase A, Ribose import ATP-binding protein, Sec-independent protein translocase subunit, serine protease, serine/threonine-protein phosphatase, sll1761-protein, slr1107-protein, sterol desaturase, threonine synthase, transcription factor, transcriptional regulator, TTC1386-protein, uroporphyrin-III C-methyltransferase, ycl027w-protein, zinc finger protein, and zinc transporter, for example of the respective polypeptide as depicted in Table II, application no. 16, column 5 and 8, or a homolog or a fragment thereof, or the respective polypeptide comprising a sequence corresponding to the consensus sequences as shown in Table IV, application no. 16, column 8, or the respective polypeptide comprising at least one polypeptide motif as depicted in Table IV, application no. 16, column 8.

A further embodiment of the present invention relates to a process for the production of coenzyme Q10, which comprises
(a) increasing or generating the activity of a amino acid acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a At1g47380-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a phenylalanine ammonia-lyase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a MADS box protein transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating the activity of a F-box protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating the activity of a CCAAT-binding transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating the activity of a CCAAT-binding transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a glycosyl transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a auxin response factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating the activity of a sterol desaturase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q10, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating the activity of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating the activity of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating the activity of a disease resistance protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a zinc finger protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a CTP synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a recombinase A non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating the activity of a enoyl-CoA hydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating the activity of a enoyl-CoA hydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a isopropylmalate isomerase large subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a AX653549-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating the activity of a AY087308-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q10, which comprises
(a) increasing or generating the activity of a threonine synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q10, which comprises
(a) increasing or generating the activity of a acetolactate synthase III large subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating the activity of a serine protease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a beta-galactosidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating the activity of a ATP-binding component of a transport system non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating the activity of a ATP-binding component of a transport system non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating the activity of a zinc transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating the activity of a zinc transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating the activity of a methylglyoxal synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating the activity of a methylglyoxal synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a b1003-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a b1097-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating the activity of a b1522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating the activity of a b1522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating the activity of a acid shock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating the activity of a acid shock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a serine/threonine-protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a b2032-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating the activity of a colanic acid biosynthesis protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating the activity of a NADH dehydrogenase I chain I non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of nicotinic acid, which comprises
(a) increasing or generating the activity of a ATP synthase subunit E non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of nicotinic acid or a composition comprising nicotinic acid in said non-human organism or in the culture medium surrounding said non-human organism.

A further embodiment of the present invention relates to a process for the production of nicotinic acid, which comprises
(a) increasing or generating the activity of a L-aspartate oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of nicotinic acid or a composition comprising nicotinic acid in said non-human organism or in the culture medium surrounding said non-human organism.

A further embodiment of the present invention relates to a process for the production of coenzyme Q9, which comprises
(a) increasing or generating the activity of a b2739-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a 2-C-methyl-D-erythritol 4-phosphate cytidylyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of lipoic acid, which comprises
(a) increasing or generating the activity of a phosphoadenosine phosphosulfate reductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lipoic acid or a composition comprising lipoic acid in said non-human organism or in the culture medium surrounding said non-human organism.

A further embodiment of the present invention relates to a process for the production of coenzyme Q9, which comprises
(a) increasing or generating the activity of a b2849-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating the activity of a Ribose import ATP-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating the activity of a b3817-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a glycerol dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating the activity of a N-acetyl-gamma-glutamylphosphate reductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating the activity of a b4029-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a gluconate transport system permease 3 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q10, which comprises
(a) increasing or generating the activity of a flavodoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q10, which comprises
(a) increasing or generating the activity of a uroporphyrin-III C-methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q10, which comprises
(a) increasing or generating the activity of a sll1761-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating the activity of a sll1761-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a cation-transporting ATPase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating the activity of a malate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating the activity of a malate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a permease protein of ABC transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a slr1107-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a fatty acid desaturase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a photosystem II protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating the activity of a glutamine amidotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q10, which comprises
(a) increasing or generating the activity of a glycerol-3-phosphate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating the activity of a glycerol-3-phosphate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a Sec-independent protein translocase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating the activity of a amino acid ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating the activity of a multiple antibiotic resistance protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a TTC1386-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a cell division control protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating the activity of a ycl027w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a halotolerance protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of nicotinic acid, which comprises
(a) increasing or generating the activity of a lipase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of nicotinic acid or a composition comprising nicotinic acid in said non-human organism or in the culture medium surrounding said non-human organism.

A further embodiment of the present invention relates to a process for the production of coenzyme Q10, which comprises
(a) increasing or generating the activity of a heat shock protein Hsp40 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating the activity of a heat shock protein Hsp40 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating the activity of a histone acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating the activity of a mitogen-activated protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q10, which comprises
(a) increasing or generating the activity of a phosphopantothenoylcysteine decarboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q10, which comprises
(a) increasing or generating the activity of a polypeptide comprising a polypeptide as depicted in the respective line in column 5 or 8 of Table II, application no. 16, or a homolog or a fragment thereof, a consensus sequence or at least one polypeptide motif as depicted in the respective line in column 8 of Table IV, application no. 16, whereby the respective line disclose in column 7 the fine chemical coenzyme Q10; or
increasing or generating the activity of an expression product of one or more nucleic acid molecule(s) comprising a polynucleotide as depicted in the respective line in column 5 or 8 of Table I, application no. 16, preferably the coding region thereof, or a homolog or a fragment thereof, whereby the respective line disclose in column 7 the fine chemical coenzyme Q10;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of coenzyme Q10, or a composition comprising coenzyme Q10 in said non-human organism or in the culture medium surrounding said non-human organism.

A further embodiment of the present invention relates to a process for the production of coenzyme Q9, which comprises
(a) increasing or generating the activity of a polypeptide comprising a polypeptide as depicted in the respective line in column 5 or 8 of Table II, application no. 16, or a homolog or a fragment thereof, a consensus sequence or at least one polypeptide motif as depicted in the respective line in column 8 of Table IV, application no. 16, whereby the respective line disclose in column 7 the fine chemical coenzyme Q9; or
increasing or generating the activity of an expression product of one or more nucleic acid molecule(s) comprising a polynucleotide as depicted in the respective line in column 5 or 8 of Table I, application no. 16, preferably the coding region thereof, or a homolog or a fragment thereof, whereby the respective line disclose in column 7 the fine chemical coenzyme Q9;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of coenzyme Q9, or a composition comprising coenzyme Q9 in said non-human organism or in the culture medium surrounding said non-human organism.

A further embodiment of the present invention relates to a process for the production of lipoic acid, which comprises
(a) increasing or generating the activity of a polypeptide comprising a polypeptide as depicted in the respective line in column 5 or 8 of Table II, application no. 16, or a homolog or a fragment thereof, a consensus sequence or at least one polypeptide motif as depicted in the respective line in column 8 of Table IV, application no. 16, whereby the respective line disclose in column 7 the fine chemical lipoic acid; or
increasing or generating the activity of an expression product of one or more nucleic acid molecule(s) comprising a polynucleotide as depicted in the respective line in column 5 or 8 of Table I, application no. 16, preferably the coding region thereof, or a homolog or a fragment thereof, whereby the respective line disclose in column 7 the fine chemical lipoic acid;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of lipoic acid, or a composition comprising lipoic acid in said non-human organism or in the culture medium surrounding said non-human organism.

A further embodiment of the present invention relates to a process for the production of nicotinic acid, which comprises
(a) increasing or generating the activity of a polypeptide comprising a polypeptide as depicted in the respective line in column 5 or 8 of Table II, application no. 16, or a homolog or a fragment thereof, a consensus sequence or at least one polypeptide motif as depicted in the respective line in column 8 of Table IV, application no. 16, whereby the respective line disclose in column 7 the fine chemical nicotinic acid; or
increasing or generating the activity of an expression product of one or more nucleic acid molecule(s) comprising a polynucleotide as depicted in the respective line in column 5 or 8 of Table I, application no. 16, preferably the coding region thereof, or a homolog or a fragment thereof, whereby the respective line disclose in column 7 the fine chemical nicotinic acid;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of nicotinic acid, or a composition comprising nicotinic acid in said non-human organism or in the culture medium surrounding said non-human organism.

A further embodiment of the present invention relates to a process for the production of coenzyme Q10, which comprises
(a) increasing or generating one or more activities selected from the group consisting of amino acid acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of At1g47380-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of phenylalanine ammonia-lyase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of MADS box protein transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating one or more activities selected from the group consisting of F-box protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating one or more activities selected from the group consisting of CCAAT-binding transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating one or more activities selected from the group consisting of CCAAT-binding transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of glycosyl transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of auxin response factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating one or more activities selected from the group consisting of sterol desaturase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q10, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating one or more activities selected from the group consisting of disease resistance protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of zinc finger protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of CTP synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of recombinase A in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating one or more activities selected from the group consisting of enoyl-CoA hydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating one or more activities selected from the group consisting of enoyl-CoA hydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of isopropylmalate isomerase large subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of AX653549-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating one or more activities selected from the group consisting of AY087308-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q10, which comprises
(a) increasing or generating one or more activities selected from the group consisting of threonine synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q10, which comprises
(a) increasing or generating one or more activities selected from the group consisting of acetolactate synthase III large subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating one or more activities selected from the group consisting of serine protease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of beta-galactosidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating one or more activities selected from the group consisting of ATP-binding component of a transport system in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating one or more activities selected from the group consisting of ATP-binding component of a transport system in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating one or more activities selected from the group consisting of zinc transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating one or more activities selected from the group consisting of zinc transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating one or more activities selected from the group consisting of methylglyoxal synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating one or more activities selected from the group consisting of methylglyoxal synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of b1003-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of b1097-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating one or more activities selected from the group consisting of b1522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating one or more activities selected from the group consisting of b1522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating one or more activities selected from the group consisting of acid shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating one or more activities selected from the group consisting of acid shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of serine/threonine-protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of b2032-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating one or more activities selected from the group consisting of colanic acid biosynthesis protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating one or more activities selected from the group consisting of NADH dehydrogenase I chain I in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of nicotinic acid, which comprises
(a) increasing or generating one or more activities selected from the group consisting of ATP synthase subunit E in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of nicotinic acid or a composition comprising nicotinic acid in said non-human organism or in the culture medium surrounding said non-human organism.

A further embodiment of the present invention relates to a process for the production of nicotinic acid, which comprises
(a) increasing or generating one or more activities selected from the group consisting of L-aspartate oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of nicotinic acid or a composition comprising nicotinic acid in said non-human organism or in the culture medium surrounding said non-human organism.

A further embodiment of the present invention relates to a process for the production of coenzyme Q9, which comprises
(a) increasing or generating one or more activities selected from the group consisting of b2739-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of 2-C-methyl-D-erythritol 4-phosphate cytidylyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of lipoic acid, which comprises
(a) increasing or generating one or more activities selected from the group consisting of phosphoadenosine phosphosulfate reductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lipoic acid or a composition comprising lipoic acid in said non-human organism or in the culture medium surrounding said non-human organism.

A further embodiment of the present invention relates to a process for the production of coenzyme Q9, which comprises
(a) increasing or generating one or more activities selected from the group consisting of b2849-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating one or more activities selected from the group consisting of Ribose import ATP-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating one or more activities selected from the group consisting of b3817-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of glycerol dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating one or more activities selected from the group consisting of N-acetyl-gamma-glutamylphosphate reductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating one or more activities selected from the group consisting of b4029-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of gluconate transport system permease 3 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q10, which comprises
(a) increasing or generating one or more activities selected from the group consisting of flavodoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q10, which comprises
(a) increasing or generating one or more activities selected from the group consisting of uroporphyrin-III C-methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q10, which comprises
(a) increasing or generating one or more activities selected from the group consisting of sll1761-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating one or more activities selected from the group consisting of sll1761-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of cation-transporting ATPase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating one or more activities selected from the group consisting of malate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating one or more activities selected from the group consisting of malate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of permease protein of ABC transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of slr1107-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of fatty acid desaturase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of photosystem II protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating one or more activities selected from the group consisting of glutamine amidotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q10, which comprises
(a) increasing or generating one or more activities selected from the group consisting of glycerol-3-phosphate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating one or more activities selected from the group consisting of glycerol-3-phosphate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of Sec-independent protein translocase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating one or more activities selected from the group consisting of amino acid ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating one or more activities selected from the group consisting of multiple antibiotic resistance protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of TTC1386-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of cell division control protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating one or more activities selected from the group consisting of ycl027w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of halotolerance protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of nicotinic acid, which comprises
(a) increasing or generating one or more activities selected from the group consisting of lipase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of nicotinic acid or a composition comprising nicotinic acid in said non-human organism or in the culture medium surrounding said non-human organism.

A further embodiment of the present invention relates to a process for the production of coenzyme Q10, which comprises
(a) increasing or generating one or more activities selected from the group consisting of heat shock protein Hsp40 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q9, which comprises
(a) increasing or generating one or more activities selected from the group consisting of heat shock protein Hsp40 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q9, which comprises
(a) increasing or generating one or more activities selected from the group consisting of histone acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

A further embodiment of the present invention relates to a process for the production of coenzyme Q10, which comprises
(a) increasing or generating one or more activities selected from the group consisting of mitogen-activated protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q10, which comprises
(a) increasing or generating one or more activities selected from the group consisting of phosphopantothenoylcysteine decarboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

A further embodiment of the present invention relates to a process for the production of coenzyme Q10, which comprises
(a1) increasing or generating the activity of a polypeptide comprising a polypeptide as depicted in the respective line in column 5 or 8 of Table II, application no. 16, or a homolog or fragment thereof, a consensus sequence or at least one polypeptide motif as depicted in column 8 of Table IV, application no. 16, whereby the respective line disclose in column 7 the fine chemical coenzyme Q10; or
increasing or generating the activity of an expression product of one or more nucleic acid molecule(s) comprising a polynucleotide as depicted in the respective line in column 5 or 8 of Table I, application no. 16, preferably the coding region thereof, or a homolog or a fragment thereof, whereby the respective line disclose in column 7 the fine chemical coenzyme Q10;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 16, or a homolog or a fragment thereof, a consensus sequence or at least one polypeptide motif as depicted in the respective line in column 8 of Table IV, application no. 16, which is joined to a transit peptide, whereby the respective line disclose in column 7 the fine chemical coenzyme Q10; or
increasing or generating the activity of an expression product of one or more nucleic acid molecule(s) comprising a polynucleotide as depicted in the respective line in column 5 or 8 of Table I, application no. 16, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical coenzyme Q10;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 16, or a homolog or a fragment thereof, a consensus sequence or at least one polypeptide motif as depicted in the respective line in column 8 of Table IV, application no. 16, whereby the respective line disclose in column 7 the fine chemical coenzyme Q10; or
increasing or generating the activity of an expression product of one or more nucleic acid molecule(s) comprising a polynucleotide as depicted in the respective line in column 5 or 8 of Table I, application no. 16, preferably the coding region thereof, or a homolog or a fragment thereof, whereby the respective line disclose in column 7 the fine chemical coenzyme Q10;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof; and
(b) growing the non-human organism under conditions which permit the production of coenzyme Q10, or a composition comprising coenzyme Q10 in said non-human organism or in the culture medium surrounding said non-human organism.

A further embodiment of the present invention relates to a process for the production of coenzyme Q9, which comprises
(a1) increasing or generating the activity of a polypeptide comprising a polypeptide as depicted in the respective line in column 5 or 8 of Table II, application no. 16, or a homolog or fragment thereof, a consensus sequence or at least one polypeptide motif as depicted in column 8 of Table IV, application no. 16, whereby the respective line disclose in column 7 the fine chemical coenzyme Q9; or
increasing or generating the activity of an expression product of one or more nucleic acid molecule(s) comprising a polynucleotide as depicted in the respective line in column 5 or 8 of Table I, application no. 16, preferably the coding region thereof, or a homolog or a fragment thereof, whereby the respective line disclose in column 7 the fine chemical coenzyme Q9;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 16, or a homolog or a fragment thereof, a consensus sequence or at least one polypeptide motif as depicted in the respective line in column 8 of Table IV, application no. 16, which is joined to a transit peptide, whereby the respective line disclose in column 7 the fine chemical coenzyme Q9; or
increasing or generating the activity of an expression product of one or more nucleic acid molecule(s) comprising a polynucleotide as depicted in the respective line in column 5 or 8 of Table I, application no. 16, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical coenzyme Q9;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 16, or a homolog or a fragment thereof, a consensus sequence or at least one polypeptide motif as depicted in the respective line in column 8 of Table IV, application no. 16, whereby the respective line disclose in column 7 the fine chemical coenzyme Q9; or
increasing or generating the activity of an expression product of one or more nucleic acid molecule(s) comprising a polynucleotide as depicted in the respective line in column 5 or 8 of Table I, application no. 16, preferably the coding region thereof, or a homolog or a fragment thereof, whereby the respective line disclose in column 7 the fine chemical coenzyme Q9;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof; and
(b) growing the non-human organism under conditions which permit the production of coenzyme Q9, or a composition comprising coenzyme Q9 in said non-human organism or in the culture medium surrounding said non-human organism.

A further embodiment of the present invention relates to a process for the production of lipoic acid, which comprises
(a1) increasing or generating the activity of a polypeptide comprising a polypeptide as depicted in the respective line in column 5 or 8 of Table II, application no. 16, or a homolog or fragment thereof, a consensus sequence or at least one polypeptide motif as depicted in column 8 of Table IV, application no. 16, whereby the respective line disclose in column 7 the fine chemical lipoic acid; or
increasing or generating the activity of an expression product of one or more nucleic acid molecule(s) comprising a polynucleotide as depicted in the respective line in column 5 or 8 of Table I, application no. 16, preferably the coding region thereof, or a homolog or a fragment thereof, whereby the respective line disclose in column 7 the fine chemical lipoic acid;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 16, or a homolog or a fragment thereof, a consensus sequence or at least one polypeptide motif as depicted in the respective line in column 8 of Table IV, application no. 16, which is joined to a transit peptide, whereby the respective line disclose in column 7 the fine chemical lipoic acid; or
increasing or generating the activity of an expression product of one or more nucleic acid molecule(s) comprising a polynucleotide as depicted in the respective line in column 5 or 8 of Table I, application no. 16, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical lipoic acid;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 16, or a homolog or a fragment thereof, a consensus sequence or at least one polypeptide motif as depicted in the respective line in column 8 of Table IV, application no. 16, whereby the respective line disclose in column 7 the fine chemical lipoic acid; or
increasing or generating the activity of an expression product of one or more nucleic acid molecule(s) comprising a polynucleotide as depicted in the respective line in column 5 or 8 of Table I, application no. 16, preferably the coding region thereof, or a homolog or a fragment thereof, whereby the respective line disclose in column 7 the fine chemical lipoic acid;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof; and
(b) growing the non-human organism under conditions which permit the production of lipoic acid, or a composition comprising lipoic acid in said non-human organism or in the culture medium surrounding said non-human organism.

A further embodiment of the present invention relates to a process for the production of nicotinic acid, which comprises
(a1) increasing or generating the activity of a polypeptide comprising a polypeptide as depicted in the respective line in column 5 or 8 of Table II, application no. 16, or a homolog or fragment thereof, a consensus sequence or at least one polypeptide motif as depicted in column 8 of Table IV, application no. 16, whereby the respective line disclose in column 7 the fine chemical nicotinic acid; or
increasing or generating the activity of an expression product of one or more nucleic acid molecule(s) comprising a polynucleotide as depicted in the respective line in column 5 or 8 of Table I, application no. 16, preferably the coding region thereof, or a homolog or a fragment thereof, whereby the respective line disclose in column 7 the fine chemical nicotinic acid;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 16, or a homolog or a fragment thereof, a consensus sequence or at least one polypeptide motif as depicted in the respective line in column 8 of Table IV, application no. 16, which is joined to a transit peptide, whereby the respective line disclose in column 7 the fine chemical nicotinic acid; or
increasing or generating the activity of an expression product of one or more nucleic acid molecule(s) comprising a polynucleotide as depicted in the respective line in column 5 or 8 of Table I, application no. 16, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical nicotinic acid;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 16, or a homolog or a fragment thereof, a consensus sequence or at least one polypeptide motif as depicted in the respective line in column 8 of Table IV, application no. 16, whereby the respective line disclose in column 7 the fine chemical nicotinic acid; or
increasing or generating the activity of an expression product of one or more nucleic acid molecule(s) comprising a polynucleotide as depicted in the respective line in column 5 or 8 of Table I, application no. 16, preferably the coding region thereof, or a homolog or a fragment thereof, whereby the respective line disclose in column 7 the fine chemical nicotinic acid;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof; and
(b) growing the non-human organism under conditions which permit the production of nicotinic acid, or a composition comprising nicotinic acid in said non-human organism or in the culture medium surrounding said non-human organism.

Accordingly, the present invention relates to a process for the production of coenzyme Q10, which comprises
(a) increasing or generating the activity of a amino acid acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a At1g47380-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a phenylalanine ammonia-lyase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a MADS box protein transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating the activity of a F-box protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating the activity of a CCAAT-binding transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating the activity of a CCAAT-binding transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a glycosyl transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a auxin response factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating the activity of a sterol desaturase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q10, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating the activity of a disease resistance protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a zinc finger protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a CTP synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a recombinase A in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating the activity of a enoyl-CoA hydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating the activity of a enoyl-CoA hydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a isopropylmalate isomerase large subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a AX653549-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating the activity of a AY087308-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q10, which comprises
(a) increasing or generating the activity of a threonine synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q10, which comprises
(a) increasing or generating the activity of a acetolactate synthase III large subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating the activity of a serine protease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a beta-galactosidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating the activity of a ATP-binding component of a transport system in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating the activity of a ATP-binding component of a transport system in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating the activity of a zinc transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating the activity of a zinc transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating the activity of a methylglyoxal synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating the activity of a methylglyoxal synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a b1003-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a b1097-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating the activity of a b1522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating the activity of a b1522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating the activity of a acid shock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating the activity of a acid shock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a serine/threonine-protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a b2032-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating the activity of a colanic acid biosynthesis protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating the activity of a NADH dehydrogenase I chain I in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of nicotinic acid, which comprises
(a) increasing or generating the activity of a ATP synthase subunit E in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of nicotinic acid or a composition comprising nicotinic acid in said non-human organism or in the culture medium surrounding said non-human organism.

Accordingly, the present invention relates to a process for the production of nicotinic acid, which comprises
(a) increasing or generating the activity of a L-aspartate oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of nicotinic acid or a composition comprising nicotinic acid in said non-human organism or in the culture medium surrounding said non-human organism.

Accordingly, the present invention relates to a process for the production of coenzyme Q9, which comprises
(a) increasing or generating the activity of a b2739-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a 2-C-methyl-D-erythritol 4-phosphate cytidylyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of lipoic acid, which comprises
(a) increasing or generating the activity of a phosphoadenosine phosphosulfate reductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lipoic acid or a composition comprising lipoic acid in said non-human organism or in the culture medium surrounding said non-human organism.

Accordingly, the present invention relates to a process for the production of coenzyme Q9, which comprises
(a) increasing or generating the activity of a b2849-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating the activity of a Ribose import ATP-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating the activity of a b3817-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a glycerol dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating the activity of a N-acetyl-gamma-glutamylphosphate reductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating the activity of a b4029-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a gluconate transport system permease 3 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q10, which comprises
(a) increasing or generating the activity of a flavodoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q10, which comprises
(a) increasing or generating the activity of a uroporphyrin-III C-methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q10, which comprises
(a) increasing or generating the activity of a sll1761-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating the activity of a sll1761-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a cation-transporting ATPase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating the activity of a malate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating the activity of a malate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a permease protein of ABC transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a slr1107-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a fatty acid desaturase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a photosystem II protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating the activity of a glutamine amidotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q10, which comprises
(a) increasing or generating the activity of a glycerol-3-phosphate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating the activity of a glycerol-3-phosphate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a Sec-independent protein translocase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating the activity of a amino acid ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating the activity of a multiple antibiotic resistance protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a TTC1386-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a cell division control protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating the activity of a ycl027w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a halotolerance protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of nicotinic acid, which comprises
(a) increasing or generating the activity of a lipase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of nicotinic acid or a composition comprising nicotinic acid in said non-human organism or in the culture medium surrounding said non-human organism.

Accordingly, the present invention relates to a process for the production of coenzyme Q10, which comprises
(a) increasing or generating the activity of a heat shock protein Hsp40 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q9, which comprises
(a) increasing or generating the activity of a heat shock protein Hsp40 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q9, which comprises
(a) increasing or generating the activity of a histone acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q9 or a composition comprising coenzyme Q9 in said non-human organism or in the culture medium surrounding said non-human organism.

Accordingly, the present invention relates to a process for the production of coenzyme Q10, which comprises
(a) increasing or generating the activity of a mitogen-activated protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q10, which comprises
(a) increasing or generating the activity of a phosphopantothenoylcysteine decarboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10 or a composition comprising coenzyme Q10 in said non-human organism or in the culture medium surrounding said non-human organism.

Accordingly, the present invention relates to a process for the production of coenzyme Q10, which comprises
(a) increasing or generating the activity of a polypeptide comprising a polypeptide as depicted in the respective line in column 5 or 8 of Table II, application no. 16, or a homolog or a fragment thereof, a consensus sequence or at least one polypeptide motif as depicted in the respective line in column 8 of Table IV, application no. 16, whereby the respective line disclose in column 7 the fine chemical coenzyme Q10; or
increasing or generating the activity of an expression product of one or more nucleic acid molecule(s) comprising a polynucleotide as depicted in the respective line in column 5 or 8 of Table I, application no. 16, preferably the coding region thereof, or a homolog or a fragment thereof, whereby the respective line disclose in column 7 the fine chemical coenzyme Q10;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of coenzyme Q10, or a composition comprising coenzyme Q10 in said non-human organism or in the culture medium surrounding said non-human organism.

Accordingly, the present invention relates to a process for the production of coenzyme Q9, which comprises
(a) increasing or generating the activity of a polypeptide comprising a polypeptide as depicted in the respective line in column 5 or 8 of Table II, application no. 16, or a homolog or a fragment thereof, a consensus sequence or at least one polypeptide motif as depicted in the respective line in column 8 of Table IV, application no. 16, whereby the respective line disclose in column 7 the fine chemical coenzyme Q9; or
increasing or generating the activity of an expression product of one or more nucleic acid molecule(s) comprising a polynucleotide as depicted in the respective line in column 5 or 8 of Table I, application no. 16, preferably the coding region thereof, or a homolog or a fragment thereof, whereby the respective line disclose in column 7 the fine chemical coenzyme Q9;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of coenzyme Q9, or a composition comprising coenzyme Q9 in said non-human organism or in the culture medium surrounding said non-human organism.

Accordingly, the present invention relates to a process for the production of lipoic acid, which comprises
(a) increasing or generating the activity of a polypeptide comprising a polypeptide as depicted in the respective line in column 5 or 8 of Table II, application no. 16, or a homolog or a fragment thereof, a consensus sequence or at least one polypeptide motif as depicted in the respective line in column 8 of Table IV, application no. 16, whereby the respective line disclose in column 7 the fine chemical lipoic acid; or
increasing or generating the activity of an expression product of one or more nucleic acid molecule(s) comprising a polynucleotide as depicted in the respective line in column 5 or 8 of Table I, application no. 16, preferably the coding region thereof, or a homolog or a fragment thereof, whereby the respective line disclose in column 7 the fine chemical lipoic acid;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of lipoic acid, or a composition comprising lipoic acid in said non-human organism or in the culture medium surrounding said non-human organism.

Accordingly, the present invention relates to a process for the production of nicotinic acid, which comprises
(a) increasing or generating the activity of a polypeptide comprising a polypeptide as depicted in the respective line in column 5 or 8 of Table II, application no. 16, or a homolog or a fragment thereof, a consensus sequence or at least one polypeptide motif as depicted in the respective line in column 8 of Table IV, application no. 16, whereby the respective line disclose in column 7 the fine chemical nicotinic acid; or
increasing or generating the activity of an expression product of one or more nucleic acid molecule(s) comprising a polynucleotide as depicted in the respective line in column 5 or 8 of Table I, application no. 16, preferably the coding region thereof, or a homolog or a fragment thereof, whereby the respective line disclose in column 7 the fine chemical nicotinic acid;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of nicotinic acid, or a composition comprising nicotinic acid in said non-human organism or in the culture medium surrounding said non-human organism.

In a further embodiment the activity of the polypeptide comprising a polypeptide as depicted in the respective line in column 5 or 8 of Table II, application no. 16, or a homolog or a fragment thereof, a consensus sequence or at least one polypeptide motif as depicted in the respective line in column 8 of Table IV, application no. 16, is increased or generated non-targeted in the above-mentioned process in a microorganism or plant or a part thereof.

In a further embodiment said polypeptide has the activity of the respective polypeptide represented by a protein comprising a polypeptide as depicted in the respective line in column 5 of Table II, application no. 16.

In a further embodiment the activity of the expression product of one or more nucleic acid molecule(s) comprising a polynucleotide as depicted in the respective line in column 5 or 8 of Table I, application no. 16, preferably the coding region thereof, or a homolog or a fragment thereof, is increased or generated non-targeted in the above-mentioned process in a microorganism or plant or a part thereof.

In a further embodiment the activity of the polypeptide comprising a polypeptide as depicted in the respective line in column 5 or 8 of Table II, application no. 16, or a homolog or a fragment thereof, a consensus sequence or at least one polypeptide motif as depicted in the respective line in column 8 of Table IV, application no. 16, is increased or generated in the above-mentioned process in an organelle preferably in plastids or mitochondria, especially in plastids, of a microorganism or plant.

In a further embodiment said polypeptide has the activity of the respective polypeptide represented by a protein comprising a polypeptide as depicted in the respective line in column 5 of Table II, application no. 16.

In a further embodiment the activity of the expression product of one or more nucleic acid molecule(s) comprising a polynucleotide as depicted in the respective line in column 5 or 8 of Table I, application no. 16, preferably the coding region thereof, or a homolog or a fragment thereof, is increased or generated in the above-mentioned process in an organelle, preferably in plasids or mitochondria, especially in plastids, of a microorganism or plant.

In a further embodiment the activity of the polypeptide comprising a polypeptide as depicted in the respective line in column 5 or 8 of Table II, application no. 16, or a homolog or a fragment thereof, a consensus sequence or at least one polypeptide motif as depicted in the respective line in column 8 of Table IV, application no. 16, is increased or generated in the above-mentioned process in the cyctosol of a cell, of a microorganism or plant.

In a further embodiment said polypeptide has the activity of the respective polypeptide represented by a protein comprising a polypeptide as depicted in the respective line in column 5 of Table II, application no. 16.

In a further embodiment the activity of the expression product of one or more nucleic acid molecule(s) comprising a polynucleotide as depicted in the respective line in column 5 or 8 of Table I, application no. 16, preferably the coding region thereof, or a homolog or a fragment thereof, is increased or generated in the above-mentioned process in the cytosol of a cell, of a microorganism or plant.

[0039.1.7.16] to [0066.1.7.16] for the disclosure of these paragraphs see [0039.1.7.7] to [0066.1.7.7] above

As mentioned above the nucleic acid sequences coding for the proteins as shown in Table II, application no. 16, column 5 or 8, or homologs or fragments thereof, are joined to a nucleic acid sequence encoding a plastidic transit peptide, e.g, if for the nucleic acid molecule in Table I, column 6 the term “plastidic” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide coding sequence is fused in frame to the nucleic acid sequence coding for proteins as shown in Table II, application no. 16, column 5 or 8, or homologs or fragments thereof, e.g., if for the nucleic acid molecule in Table I, column 6 the term “plastidic” is indicated.

[0068.1.7.16] to [0072.1.7.16] for the disclosure of these paragraphs see [0068.1.7.7] to [0072.1.7.7] above

As mentioned above the nucleic acid sequences coding for the proteins as shown in the respective line in Table II, application no. 16, column 5 or 8, or homologs or fragments thereof, are joined to a nucleic acid sequence encoding a mitochondric transit peptide, e.g., if for the respective nucleic acid molecule in Table I, column 6 the term “mitochondric” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in the respective line in Table II, application no. 16, column 5 or 8, or homologs or fragments thereof, e.g., if for the nucleic acid molecule in the respective line in Table I, column 6 the term “mitochondric” is indicated.

for the disclosure of this paragraph see [0074.1.7.7] above

for the disclosure of this paragraph see [0075.1.7.7] above

Nucleic acid sequences coding for the transit peptides may be chemically synthesized either in part or wholly according to structure of transit peptide sequences disclosed in the prior art. Said natural or chemically synthesized sequences can be directly linked to the sequences encoding the mature protein or via a linker nucleic acid sequence, which may be typically less than 500 base pairs, preferably less than 450, 400, 350, 300, 250 or 200 base pairs, more preferably less than 150, 100, 90, 80, 70, 60, 50, 40 or 30 base pairs and most preferably less than 25, 20, 15, 12, 9, 6 or 3 base pairs in length and are in frame to the coding sequence. Furthermore favorable nucleic acid sequences encoding transit peptides may comprise sequences derived from more than one biological and/or chemical source and may include a nucleic acid sequence derived from the amino-terminal region of the mature protein, which in its native state is linked to the transit peptide. In a preferred embodiment of the invention said amino-terminal region of the mature protein is typically less than 150 amino acids, preferably less than 140, 130, 120, 110, 100 or 90 amino acids, more preferably less than 80, 70, 60, 50, 40, 35, 30, 25 or 20 amino acids and most preferably less than 19, 18, 17, 16, 15, 14, 13, 12, 11 or 10 amino acids in length. But even shorter or longer stretches are also possible. In addition target sequences, which facilitate the transport of proteins to other cell compartments such as the vacuole, endoplasmic reticulum, golgi complex, glyoxysomes, peroxisomes, mitochondria (in case of a plastidal transit peptide) or plastids, especially chloroplasts (in case of a mitochondric transit peptide) may be also part of the inventive nucleic acid sequence. The proteins translated from said inventive nucleic acid sequences are a kind of fusion proteins that means the nucleic acid sequences encoding the plastidal transit peptide for example the ones shown in Table a, preferably the last one of the Table a are joint to the respective nucleic acid sequences shown in Table I, application no. 16, columns 5 or 8, especially the coding region thereof, or homologs or fragments thereof, in case of nucleic acid sequences being expressed in plastids, and that means the nucleic acid sequences encoding the mitochondric transit peptide for example the ones shown in Table b, preferably the last one of the Table b are joint to the respective nucleic acid sequences shown in Table I, application no. 16, columns 5 or 8, especially the coding region thereof, or homologs or fragments thereof, in case of nucleic acid sequences being expressed in mitochondria. The person skilled in the art is able to join said sequences in a functional manner. Advantageously the transit peptide part is cleaved off from the protein part shown in Table II, application no. 16, columns 5 or 8, or a homolog or fragment thereof, during or after the transport into the organelles, preferably into the plastids or mitochondria, respectively.

All products of the cleavage of the preferred transit peptide shown in the last line of Table a have preferably the N-terminal amino acid sequences QIA CSS or QIA EFQLTT in front of the start methionine of the respective protein mentioned in Table II, application no. 16, column 5 or 8. or homologs or fragments thereof. Other short amino acid sequences of an range of 1 to 20 amino acids preferable 2 to 15 amino acids, more preferable 3 to 10 amino acids most preferably 4 to 8 amino acids are also possible in front of the start methionine of the protein mentioned in Table II, application no. 16, columns 5 or 8, or homologs or fragments thereof. In case of the amino acid sequence QIA CSS the three amino acids in front of the start methionine are stemming from the LIC (=ligatation independent cloning) cassette. Said short amino acid sequence is preferred in the case of the expression of E. coli genes. In case of the amino acid sequence QIA EFQLTT the six amino acids in front of the start methionine are stemming from the LIC cassette. Said short amino acid sequence is preferred in the case of the expression of S. cerevisiae genes. The skilled worker knowns that other short sequences are also useful in the expression of the respective nucleic acid molecule comprising a polynucleotide as depicted in Table I, application no. 16, column 5 or 8, preferably the coding region thereof, or a homolog or a fragment thereof. Furthermore the skilled worker is aware of the fact that there is not a need for such short sequences in the expression of the genes.

for the disclosure of this paragraph see [0077.1.7.7] above

for the disclosure of this paragraph see [0078.1.7.7] above

Alternatively to the targeting of the respective sequences shown in Table II, application no. 16, columns 5 or 8, or homologs or fragments thereof, preferably of sequences in general encoded in the nucleus with the aid of the targeting sequences mentioned for example in Table a alone, Table b alone, or in combination with other targeting sequences preferably into plastids or mitochondria, respectively, the nucleic acids of the invention can directly be introduced into the plastidal or mitochondrial genome. Therefore in a preferred embodiment the respective nucleic acid sequences shown in the respective line in Table I, application no. 16, columns 5 or 8, preferably the coding region thereof, or homologs or fragments thereof, are directly introduced and expressed in plastids e.g., if for the nucleic acid molecule in the respective line in Table I, column 6, the term “plastidic” is indicated, or are directly introduced and expressed in mitochondria e.g., if for the nucleic acid molecule in the respective line in Table I, column 6 the term “mitochondric” is indicated.

The term “introduced” in the context of this specification shall mean the insertion of a nucleic acid sequence into the organism by means of a “transfection”, “transduction” or preferably by “transformation”.

A plastid, such as a chloroplast, or a mitochondrion, has been “transformed” by an exogenous (preferably foreign) nucleic acid sequence if a nucleic acid sequence has been introduced into the plastid or mitochondrion that means that this sequence has crossed the membrane or the membranes of the plastid or of the mitochondrion. The exogenous, preferably foreign, DNA may be integrated (covalently linked) into plastid or mitochondrial DNA making up the genome of the plastid or mitochondrion, or it may remain unintegrated (e.g., by including a chloroplast origin of replication). “Stably” integrated DNA sequences are those, which are inherited through plastid or mitochondrion replication, thereby transferring new plastids or mitochondria, with the features of the integrated DNA sequence to the progeny.

[0080.1.7.16] to [0083.1.7.16] for the disclosure of these paragraphs see [0080.1.7.7] to [0083.1.7.7] above

For the process of the present invention it may be of great advantage that by transforming the plastids the intraspecies specific transgene flow is blocked, because a lot of species such as corn, cotton and rice have a strict maternal inheritance of plastids. By placing the respective genes specified in the respective line in Table I, application no. 16, columns 5 or 8, preferably the coding region, or homologs or fragments thereof, e.g. if for the nucleic acid molecule in the respective line in column 6 of Table I the term “plastidic” is indicated, in the plastids of plants, these genes will not be present in the pollen of said plants.

A further preferred embodiment of the invention relates to the use of so called “chloroplast localization sequences”, in which a first RNA sequence or molecule is capable of transporting or “chaperoning” a second RNA sequence, such as a RNA sequence transcribed from the sequences depicted in the respective line in Table I, application no. 16, columns 5 or 8, preferably the coding region thereof, or a homolog or a fragment thereof, or a sequence encoding a protein, as depicted in the respective line in Table II, application no. 16, columns 5 and 8, or a homolog or a fragment thereof, from an external environment inside a cell or outside a plastid into a chloroplast. In one embodiment the chloroplast localization signal is substantially similar or complementary to a complete or intact viroid sequence. The chloroplast localization signal may be encoded by a DNA sequence, which is transcribed into the chloroplast localization RNA. The term “viroid” refers to a naturally occurring single stranded RNA molecule (Flores, C. R. Acad. Sci. III. 324 (10), 943 (2001)). Viroids usually contain about 200-500 nucleotides and generally exist as circular molecules. Examples of viroids that contain chloroplast localization signals include but are not limited to ASBVd, PLMVd, CChMVd and ELVd. The viroid sequence or a functional part of it can be fused to the sequences depicted in the respective line in Table I, application no. 16, columns 5 or 8, preferably the coding region thereof, or a homolog or a fragment thereof, or a sequence encoding a protein, as depicted in the respective line in Table II, application no. 16, columns 5 or 8, or a homolog or a fragment thereof, in such a manner that the viroid sequence transports a sequence transcribed from a sequence as depicted in the respective line in Table I, application no. 16 columns 5 or 8, preferably the coding region thereof, or a homolog or a fragment thereof, or a sequence encoding a protein as depicted in the respective line in Table II, application no. 16 columns 5 or 8, or a homolog or a fragment thereof, into the chloroplasts, e.g. if for the nucleic acid molecule in column 6 of Table I the term “plastidic” is indicated. A preferred embodiment uses a modified ASBVd (Navarro et al., Virology. 268 (1), 218 (2000)).

In a further specific embodiment the protein to be expressed in the plastids such as the proteins depicted in the respective line in Table II, application no. 16, columns 5 or 8, or a homolog or a fragment thereof, e.g. if for the nucleic acid molecule in the respective line in column 6 of Table I the term “plastidic” is indicated, are encoded by different nucleic acids. Such a method is disclosed in WO 2004/040973, which shall be incorporated by reference. WO 2004/040973 teaches a method, which relates to the translocation of an RNA corresponding to a gene or gene fragment into the chloroplast by means of a chloroplast localization sequence.

The genes, which should be expressed in the plant or plant cells, are split into nucleic acid fragments, which are introduced into different compartments in the plant e.g. the nucleus, the plastids and/or mitochondria. Additionally plant cells are described in which the chloroplast contains a ribozyme fused at one end to an RNA encoding a fragment of a protein used in the inventive process such that the ribozyme can trans-splice the translocated fusion RNA to the RNA encoding the gene fragment to form and as the case may be reunite the nucleic acid fragments to an intact mRNA encoding a functional protein for example as disclosed in the respective line in Table II, application no. 16, columns 5 or 8, or a homolog or a fragment thereof.

In a preferred embodiment of the invention the nucleic acid sequences as shown in the respective line in Table I, application no. 16, columns 5 or 8, preferably the coding region thereof, or homologs or fragments thereof, e.g. if for the nucleic acid molecule in the respective line in column 6 of Table I the term “plastidic” is indicated, used in the inventive process are transformed into plastids, which are metabolically active. Those plastids should preferably maintain at a high copy number in the plant or plant tissue of interest, most preferably the chloroplasts found in green plant tissues, such as leaves or cotyledons or in seeds.

For a good expression in the plastids the nucleic acid sequences as shown in the respective line in Table I, application no. 16, columns 5 or 8, preferably the coding region thereof, or homologs or fragments thereof, e.g. if for the nucleic acid molecule in the respective line in column 6 of Table I the term “plastidic” is indicated, are introduced into an expression cassette using preferably a promoter and terminater, which are active in plastids preferably a chloroplast promoter. Examples of such promoters include the psbA promoter from the gene from spinach or pea, the rbcL promoter, and the atpB promoter from corn.

[0089.1.7.16] to [0092.1.7.16] for the disclosure of these paragraphs see [0089.1.7.7] to [0092.1.7.7] above

Advantageously the process for the production of the fine chemical coenzyme Q10, coenzyme Q9, lipoic acid, and/or nicotinic acid leads to an enhanced production of the respective fine chemical. The terms “enhanced” or “increase” mean at least a 10%, 20%, 30%, 40% or 50%, preferably at least 60%, 70%, 80%, 90% or 100%, more preferably 150%, 200%, 300%, 400% or 500% higher production of the respective fine chemical coenzyme Q10, coenzyme Q9, lipoic acid, and/or nicotinic acid in comparison to the wild-type as defined above, e.g. that means in comparison to a non-human organism without the aforementioned modification of the activity of a protein as shown in the respective line in Table II, application no. 16, column 5 or 8, or a fragment or a homolog thereof. The modification of the activity of a protein as shown in the respective line in Table II, application no. 16, column 5 or 8, or a homolog or a fragment thereof, or their combination can be achieved by joining the protein to a respective transit peptide, e.g. if for the respective encoding nucleic acid molecule in column 6 of Table I the term “plastidic” or “mitochondric” is indicated.

Surprisingly it was found, that the transgenic expression of a protein as shown in the respective line in Table II, application no. 16, column 5, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, conferred a production of or an increase in coenzyme Q10, coenzyme Q9, lipoic acid, and/or nicotinic acid,respectively, to the transgenic non-human organism as compared to a corresponding non-transformed wild type.

In one embodiment the transgenic expression of a protein as shown in the respective line in Table II, application no. 16, column 5 or 8, or a homolog or a fragment thereof, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, confers a production of or an increase in coenzyme Q10, coenzyme Q9, lipoic acid, and/or nicotinic acid, respectively, to the transgenic non-human organism as compared to a corresponding non-transformed wild type.

Accordingly, in one embodiment, an increase of coenzyme Q10 in a non-human organism, as compared to a corresponding non-transformed wild type non-human organism, is conferred in the process of the invention, if the activity of a polypeptide showing the activity of a amino acid acetyltransferase, or if the activity of the polypeptide AAC43185, preferably represented by SEQ ID NO. 17357, or a homolog or fragment thereof, or if the activity of a polypeptide encoded by a nucleic acid molecule comprising the nucleic acid SEQ ID NO. 17356, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 17356 or polypeptide SEQ ID NO. 17357, respectively, or a homolog or a fragment thereof, is increased or generated, or if the activity amino acid acetyltransferase is increased or generated in a non-human organism, like a microorganism or a plant cell, plant or part thereof, especially with plastidic localization, whereby the respective line disclose in column 7 the fine chemical coenzyme Q10. For example, an increase of the coenzyme Q10 of at least 1 percent, particularly in a range of 67 to 296-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a transcriptional regulator, or if the activity of the polypeptide At1g43850, preferably represented by SEQ ID NO. 18071, or a homolog or fragment thereof, or if the activity of a polypeptide encoded by a nucleic acid molecule comprising the nucleic acid SEQ ID NO. 18070, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 18070 or polypeptide SEQ ID NO. 18071, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 37 to 123-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a At1g47380-protein, or if the activity of the polypeptide At1g47380, preferably represented by SEQ ID NO. 18123, or a homolog or fragment thereof, or if the activity of a polypeptide encoded by a nucleic acid molecule comprising the nucleic acid SEQ ID NO. 18122, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 18122 or polypeptide SEQ ID NO. 18123, respectively, or a homolog or a fragment thereof, is increased or generated, or if the activity At1g47380-protein is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 36 to 98-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a protein phosphatase, or if the activity of the polypeptide At1g72770, preferably represented by SEQ ID NO. 19420, or a homolog or fragment thereof, or if the activity of a polypeptide encoded by a nucleic acid molecule comprising the nucleic acid SEQ ID NO. 19419, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 19419 or polypeptide SEQ ID NO. 19420, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 52 to 65-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a phenylalanine ammonia-lyase, or if the activity of the polypeptide At2g37040, preferably represented by SEQ ID NO. 133043, or a homolog or fragment thereof, or if the activity of a polypeptide encoded by a nucleic acid molecule comprising the nucleic acid SEQ ID NO. 133042, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 133042 or polypeptide SEQ ID NO. 133043, respectively, or a homolog or a fragment thereof, is increased or generated, or if the activity phenylalanine ammonia-lyase is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 39 to 85-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a MADS box protein transcription factor, or if the activity of the polypeptide At2g42830, preferably represented by SEQ ID NO. 108728, or a homolog or fragment thereof, or if the activity of a polypeptide encoded by a nucleic acid molecule comprising the nucleic acid SEQ ID NO. 108727, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 108727 or polypeptide SEQ ID NO. 108728, respectively, or a homolog or a fragment thereof, is increased or generated, or if the activity MADS box protein transcription factor is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 37 to 91-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a glutaredoxin, or if the activity of the polypeptide At2g47880, preferably represented by SEQ ID NO. 21160, or a homolog or fragment thereof, or if the activity of a polypeptide encoded by a nucleic acid molecule comprising the nucleic acid SEQ ID NO. 21159, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 21159 or polypeptide SEQ ID NO. 21160, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical coenzyme Q10. For example, an increase of the coenzyme Q10 of at least 1 percent, particularly in a range of 79 to 87-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a heat shock transcription factor, or if the activity of the polypeptide At3g02990, preferably represented by SEQ ID NO. 68658, or a homolog or fragment thereof, or if the activity of a polypeptide encoded by a nucleic acid molecule comprising the nucleic acid SEQ ID NO. 68657, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 68657 or polypeptide SEQ ID NO. 68658, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 40 to 148-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a pyruvate kinase, or if the activity of the polypeptide At3g04050, preferably represented by SEQ ID NO. 21498, or a homolog or fragment thereof, or if the activity of a polypeptide encoded by a nucleic acid molecule comprising the nucleic acid SEQ ID NO. 21497, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 21497 or polypeptide SEQ ID NO. 21498, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line disclose in column 7 the fine chemical coenzyme Q10. For example, an increase of the coenzyme Q10 of at least 1 percent, particularly in a range of 85 to 142-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a F-box protein, or if the activity of the polypeptide At3g16820, preferably represented by SEQ ID NO. 120953, or a homolog or fragment thereof, or if the activity of a polypeptide encoded by a nucleic acid molecule comprising the nucleic acid SEQ ID NO. 120952, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 120952 or polypeptide SEQ ID NO. 120953, respectively, or a homolog or a fragment thereof, is increased or generated, or if the activity F-box protein is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 45 to 114-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a CCAAT-binding transcription factor, or if the activity of the polypeptide At3g20910, preferably represented by SEQ ID NO. 22922, or a homolog or fragment thereof, or if the activity of a polypeptide encoded by a nucleic acid molecule comprising the nucleic acid SEQ ID NO. 22921, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 22921 or polypeptide SEQ ID NO. 22922, respectively, or a homolog or a fragment thereof, is increased or generated, or if the activity CCAAT-binding transcription factor is increased or generated in a non-human organism, like a microorganism or a plant cell, plant or part thereof, especially with non-targeted localization, whereby the respective line disclose in column 7 the fine chemical coenzyme Q10. For example, an increase of the coenzyme Q10 of at least 1 percent, particularly in a range of 67 to 438-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a CCAATbinding transcription factor, or if the activity of the polypeptide At3g20910, preferably represented by SEQ ID NO. 22922, or a homolog or fragment thereof, or if the activity of a polypeptide encoded by a nucleic acid molecule comprising the nucleic acid SEQ ID NO. 22921, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 22921 or polypeptide SEQ ID NO. 22922, respectively, or a homolog or a fragment thereof, is increased or generated, or if the activity CCAAT-binding transcription factor is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 37 to 70-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a glycosyl transferase, or if the activity of the polypeptide At3g27540, preferably represented by SEQ ID NO. 68778, or a homolog or fragment thereof, or if the activity of a polypeptide encoded by a nucleic acid molecule comprising the nucleic acid SEQ ID NO. 68777, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 68777 or polypeptide SEQ ID NO. 68778, respectively, or a homolog or a fragment thereof, is increased or generated, or if the activity glycosyl transferase is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 36 to 86-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a auxin response factor, or if the activity of the polypeptide At3g61830, preferably represented by SEQ ID NO. 2368, or a homolog or fragment thereof, or if the activity of a polypeptide encoded by a nucleic acid molecule comprising the nucleic acid SEQ ID NO. 2367, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 2367 or polypeptide SEQ ID NO. 2368, respectively, or a homolog or a fragment thereof, is increased or generated, or if the activity auxin response factor is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 36 to 177-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical coenzyme Q10. For example, an increase of the coenzyme Q10 of at least 1 percent, particularly in a range of 52 to 102-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 52 to 91-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a sterol desaturase, or if the activity of the polypeptide At4g12110, preferably represented by SEQ ID NO. 131209, or a homolog or fragment thereof, or if the activity of a polypeptide encoded by a nucleic acid molecule comprising the nucleic acid SEQ ID NO. 131208, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 131208 or polypeptide SEQ ID NO. 131209, respectively, or a homolog or a fragment thereof, is increased or generated, or if the activity sterol desaturase is increased or generated in a non-human organism, like a microorganism or a plant cell, plant or part thereof, especially with non-targeted localization, whereby the respective line disclose in column 7 the fine chemical coenzyme Q10. For example, an increase of the coenzyme Q10 of at least 1 percent, particularly in a range of 72 to 96-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical coenzyme Q10. For example, an increase of the coenzyme Q10 of at least 1 percent, particularly in a range of 89 to 174 percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 53 to 105-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a cyclin D , or if the activity of the polypeptide At4g34160, preferably represented by SEQ ID NO. 24312, or a homolog or fragment thereof, or if the activity of a polypeptide encoded by a nucleic acid molecule comprising the nucleic acid SEQ ID NO. 24311, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 24311 or polypeptide SEQ ID NO. 24312, respectively, or a homolog or a fragment thereof, is increased or generated, or if the activity cyclin D is increased or generated in a non-human organism, like a microorganism or a plant cell, plant or part thereof, especially with non-targeted localization, whereby the respective line disclose in column 7 the fine chemical coenzyme Q10. For example, an increase of the coenzyme Q10 of at least 1 percent, particularly in a range of 78 to 175-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a cyclin D , or if the activity of the polypeptide At4g34160, preferably represented by SEQ ID NO. 24312, or a homolog or fragment thereof, or if the activity of a polypeptide encoded by a nucleic acid molecule comprising the nucleic acid SEQ ID NO. 24311, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 24311 or polypeptide SEQ ID NO. 24312, respectively, or a homolog or a fragment thereof, is increased or generated, or if the activity cyclin D is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 38 to 131-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line disclose in column 7 the fine chemical coenzyme Q10. For example, an increase of the coenzyme Q10 of at least 1 percent, particularly in a range of 72 to 393-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 36 to 244-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a heat shock transcription factor, or if the activity of the polypeptide At5g03720, preferably represented by SEQ ID NO. 24439, or a homolog or fragment thereof, or if the activity of a polypeptide encoded by a nucleic acid molecule comprising the nucleic acid SEQ ID NO. 24438, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 24438 or polypeptide SEQ ID NO. 24439, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 46 to 53-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a monothiol glutaredoxin, or if the activity of the polypeptide At5g18600, preferably represented by SEQ ID NO. 4905, or a homolog or fragment thereof, or if the activity of a polypeptide encoded by a nucleic acid molecule comprising the nucleic acid SEQ ID NO. 4904, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 4904 or polypeptide SEQ ID NO. 4905, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical coenzyme Q10. For example, an increase of the coenzyme Q10 of at least 1 percent, particularly in a range of 57 to 63 percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a disease resistance protein, or if the activity of the polypeptide At5g45060, preferably represented by SEQ ID NO. 102224, or a homolog or fragment thereof, or if the activity of a polypeptide encoded by a nucleic acid molecule comprising the nucleic acid SEQ ID NO. 102223, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 102223 or polypeptide SEQ ID NO. 102224, respectively, or a homolog or a fragment thereof, is increased or generated, or if the activity disease resistance protein is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 41 to 53-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a protein phosphatase, or if the activity of the polypeptide At5g59220, preferably represented by SEQ ID NO. 25499, or a homolog or fragment thereof, or if the activity of a polypeptide encoded by a nucleic acid molecule comprising the nucleic acid SEQ ID NO. 25498, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 25498 or polypeptide SEQ ID NO. 25499, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 36 to 97-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a zinc finger protein, or if the activity of the polypeptide At5g64920, preferably represented by SEQ ID NO. 5494, or a homolog or fragment thereof, or if the activity of a polypeptide encoded by a nucleic acid molecule comprising the nucleic acid SEQ ID NO. 5493, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 5493 or polypeptide SEQ ID NO. 5494, respectively, or a homolog or a fragment thereof, is increased or generated, or if the activity zinc finger protein is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 36 to 114-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a transcriptional regulator, or if the activity of the polypeptide AvinDRAFT_{—}3209, preferably represented by SEQ ID NO. 29287, or a homolog or fragment thereof, or if the activity of a polypeptide encoded by a nucleic acid molecule comprising the nucleic acid SEQ ID NO. 29286, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 29286 or polypeptide SEQ ID NO. 29287, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 52 to 101-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a CTP synthase, or if the activity of the polypeptide AvinDRAFT_{—}3605, preferably represented by SEQ ID NO. 31027, or a homolog or fragment thereof, or if the activity of a polypeptide encoded by a nucleic acid molecule comprising the nucleic acid SEQ ID NO. 31026, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 31026 or polypeptide SEQ ID NO. 31027, respectively, or a homolog or a fragment thereof, is increased or generated, or if the activity CTP synthase is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 46 to 176-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a recombinase A, or if the activity of the polypeptide AvinDRAFT_{—}3629, preferably represented by SEQ ID NO. 114232, or a homolog or fragment thereof, or if the activity of a polypeptide encoded by a nucleic acid molecule comprising the nucleic acid SEQ ID NO. 114231, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 114231 or polypeptide SEQ ID NO. 114232, respectively, or a homolog or a fragment thereof, is increased or generated, or if the activity recombinase A is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 42 to 171-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a enoyl-CoA hydratase, or if the activity of the polypeptide AvinDRAFT_{—}4420, preferably represented by SEQ ID NO. 73039, or a homolog or fragment thereof, or if the activity of a polypeptide encoded by a nucleic acid molecule comprising the nucleic acid SEQ ID NO. 73038, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 73038 or polypeptide SEQ ID NO. 73039, respectively, or a homolog or a fragment thereof, is increased or generated, or if the activity enoyl-CoA hydratase is increased or generated in a non-human organism, like a microorganism or a plant cell, plant or part thereof, especially with non-targeted localization, whereby the respective line disclose in column 7 the fine chemical coenzyme Q10. For example, an increase of the coenzyme Q10 of at least 1 percent, particularly in a range of 109 to 257-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a enoyl-CoA hydratase, or if the activity of the polypeptide AvinDRAFT_{—}4420, preferably represented by SEQ ID NO. 73039, or a homolog or fragment thereof, or if the activity of a polypeptide encoded by a nucleic acid molecule comprising the nucleic acid SEQ ID NO. 73038, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 73038 or polypeptide SEQ ID NO. 73039, respectively, or a homolog or a fragment thereof, is increased or generated, or if the activity enoyl-CoA hydratase is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 98 to 220-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a isopropylmalate isomerase large subunit, or if the activity of the polypeptide AvinDRAFT_{—}4847, preferably represented by SEQ ID NO. 102942, or a homolog or fragment thereof, or if the activity of a polypeptide encoded by a nucleic acid molecule comprising the nucleic acid SEQ ID NO. 102941, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 102941 or polypeptide SEQ ID NO. 102942, respectively, or a homolog or a fragment thereof, is increased or generated, or if the activity isopropylmalate isomerase large subunit is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 36 to 79-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a hydrolase, or if the activity of the polypeptide AvinDRAFT_{—}5103, preferably represented by SEQ ID NO. 6511, or a homolog or fragment thereof, or if the activity of a polypeptide encoded by a nucleic acid molecule comprising the nucleic acid SEQ ID NO. 6510, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 6510 or polypeptide SEQ ID NO. 6511, respectively, or a homolog or a fragment thereof, is increased or generated, or if the activity hydrolase is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 195 to 240-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a AX653549-protein, or if the activity of the polypeptide AX653549, preferably represented by SEQ ID NO. 34302, or a homolog or fragment thereof, or if the activity of a polypeptide encoded by a nucleic acid molecule comprising the nucleic acid SEQ ID NO. 34301, preferably the coding region thereof, or a homolog or fragment thereof, e.g. derived from Oryza sativa, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 34301 or polypeptide SEQ ID NO. 34302, respectively, or a homolog or a fragment thereof, is increased or generated, or if the activity AX653549-protein is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 44 to 55-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a AY087308-protein, or if the activity of the polypeptide AY087308, preferably represented by SEQ ID NO. 34603, or a homolog or fragment thereof, or if the activity of a polypeptide encoded by a nucleic acid molecule comprising the nucleic acid SEQ ID NO. 34602, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 34602 or polypeptide SEQ ID NO. 34603, respectively, or a homolog or a fragment thereof, is increased or generated, or if the activity AY087308-protein is increased or generated in a non-human organism, like a microorganism or a plant cell, plant or part thereof, especially with non-targeted localization, whereby the respective line disclose in column 7 the fine chemical coenzyme Q10. For example, an increase of the coenzyme Q10 of at least 1 percent, particularly in a range of 60 to 92-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a threonine synthase, or if the activity of the polypeptide B0004, preferably represented by SEQ ID NO. 34890, or a homolog or fragment thereof, or if the activity of a polypeptide encoded by a nucleic acid molecule comprising the nucleic acid SEQ ID NO. 34889, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 34889 or polypeptide SEQ ID NO. 34890, respectively, or a homolog or a fragment thereof, is increased or generated, or if the activity threonine synthase is increased or generated in a non-human organism, like a microorganism or a plant cell, plant or part thereof, especially with plastidic localization, whereby the respective line disclose in column 7 the fine chemical coenzyme Q10. For example, an increase of the coenzyme Q10 of at least 1 percent, particularly in a range of 70 to 558-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a acetolactate synthase III large subunit, or if the activity of the polypeptide B0077, preferably represented by SEQ ID NO. 137835, or a homolog or fragment thereof, or if the activity of a polypeptide encoded by a nucleic acid molecule comprising the nucleic acid SEQ ID NO. 137834, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 137834 or polypeptide SEQ ID NO. 137835, respectively, or a homolog or a fragment thereof, is increased or generated, or if the activity acetolactate synthase III large subunit is increased or generated in a non-human organism, like a microorganism or a plant cell, plant or part thereof, especially with plastidic localization, whereby the respective line disclose in column 7 the fine chemical coenzyme Q10. For example, an increase of the coenzyme Q10 of at least 1 percent, particularly in a range of 21 to 34-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a serine protease, or if the activity of the polypeptide B0161, preferably represented by SEQ ID NO. 7082, or a homolog or fragment thereof, or if the activity of a polypeptide encoded by a nucleic acid molecule comprising the nucleic acid SEQ ID NO. 7081, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 7081 or polypeptide SEQ ID NO. 7082, respectively, or a homolog or a fragment thereof, is increased or generated, or if the activity serine protease is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 38 to 146-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a beta-galactosidase, or if the activity of the polypeptide B0344, preferably represented by SEQ ID NO. 35734, or a homolog or fragment thereof, or if the activity of a polypeptide encoded by a nucleic acid molecule comprising the nucleic acid SEQ ID NO. 35733, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 35733 or polypeptide SEQ ID NO. 35734, respectively, or a homolog or a fragment thereof, is increased or generated, or if the activity beta-galactosidase is increased or generated in a non-human organism, like a microorganism or a plant cell, plant or part thereof, especially with plastidic localization, whereby the respective line disclose in column 7 the fine chemical coenzyme Q9. For example, an increase of the coenzyme Q9 of at least 1 percent, particularly in a range of 17 to 59-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a ATP-binding component of a transport system, or if the activity of the polypeptide B0449, preferably represented by SEQ ID NO. 7334, or a homolog or fragment thereof, or if the activity of a polypeptide encoded by a nucleic acid molecule comprising the nucleic acid SEQ ID NO. 7333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 7333 or polypeptide SEQ ID NO. 7334, respectively, or a homolog or a fragment thereof, is increased or generated, or if the activity ATP-binding component of a transport system is increased or generated in a non-human organism, like a microorganism or a plant cell, plant or part thereof, especially with non-targeted localization, whereby the respective line disclose in column 7 the fine chemical coenzyme Q10. For example, an increase of the coenzyme Q10 of at least 1 percent, particularly in a range of 69 to 266-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a ATP-binding component of a transport system, or if the activity of the polypeptide B0449, preferably represented by SEQ ID NO. 7334, or a homolog or fragment thereof, or if the activity of a polypeptide encoded by a nucleic acid molecule comprising the nucleic acid SEQ ID NO. 7333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 7333 or polypeptide SEQ ID NO. 7334, respectively, or a homolog or a fragment thereof, is increased or generated, or if the activity ATP-binding component of a transport system is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 71 to 209-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a zinc transporter, or if the activity of the polypeptide B0752, preferably represented by SEQ ID NO. 36115, or a homolog or fragment thereof, or if the activity of a polypeptide encoded by a nucleic acid molecule comprising the nucleic acid SEQ ID NO. 36114, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 36114 or polypeptide SEQ ID NO. 36115, respectively, or a homolog or a fragment thereof, is increased or generated, or if the activity zinc transporter is increased or generated in a non-human organism, like a microorganism or a plant cell, plant or part thereof, especially with non-targeted localization, whereby the respective line disclose in column 7 the fine chemical coenzyme Q10. For example, an increase of the coenzyme Q10 of at least 1 percent, particularly in a range of 98 to 188-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a zinc transporter, or if the activity of the polypeptide B0752, preferably represented by SEQ ID NO. 36115, or a homolog or fragment thereof, or if the activity of a polypeptide encoded by a nucleic acid molecule comprising the nucleic acid SEQ ID NO. 36114, preferably the coding region thereof, or a homolog or fragment thereof, e.g. derived from Escherichia coli, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 36114 or polypeptide SEQ ID NO. 36115, respectively, or a homolog or a fragment thereof, is increased or generated, or if the activity zinc transporter is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 53 to 96-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a methylglyoxal synthase, or if the activity of the polypeptide B0963, preferably represented by SEQ ID NO. 36671, or a homolog or fragment thereof, or if the activity of a polypeptide encoded by a nucleic acid molecule comprising the nucleic acid SEQ ID NO. 36670, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 36670 or polypeptide SEQ ID NO. 36671, respectively, or a homolog or a fragment thereof, is increased or generated, or if the activity methylglyoxal synthase is increased or generated in a non-human organism, like a microorganism or a plant cell, plant or part thereof, especially with non-targeted localization, whereby the respective line disclose in column 7 the fine chemical coenzyme Q10. For example, an increase of the coenzyme Q10 of at least 1 percent, particularly in a range of 54 to 113-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a methylglyoxal synthase, or if the activity of the polypeptide B0963, preferably represented by SEQ ID NO. 36671, or a homolog or fragment thereof, or if the activity of a polypeptide encoded by a nucleic acid molecule comprising the nucleic acid SEQ ID NO. 36670, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 36670 or polypeptide SEQ ID NO. 36671, respectively, or a homolog or a fragment thereof, is increased or generated, or if the activity methylglyoxal synthase is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 46 to 60-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a b1003-protein, or if the activity of the polypeptide B1003, preferably represented by SEQ ID NO. 7942, or a homolog or fragment thereof, or if the activity of a polypeptide encoded by a nucleic acid molecule comprising the nucleic acid SEQ ID NO. 7941, preferably the coding region thereof, or a homolog or fragment thereof, e.g. derived from Escherichia coli, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 7941 or polypeptide SEQ ID NO. 7942, respectively, or a homolog or a fragment thereof, is increased or generated, or if the activity b1003-protein is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 40 to 287-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a b1097-protein, or if the activity of the polypeptide B1097, preferably represented by SEQ ID NO. 138257, or a homolog or fragment thereof, or if the activity of a polypeptide encoded by a nucleic acid molecule comprising the nucleic acid SEQ ID NO. 138256, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 138256 or polypeptide SEQ ID NO. 138257, respectively, or a homolog or a fragment thereof, is increased or generated, or if the activity b1097-protein is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 40 to 68-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a b1522-protein, or if the activity of the polypeptide B1522, preferably represented by SEQ ID NO. 7948, or a homolog or fragment thereof, or if the activity of a polypeptide encoded by a nucleic acid molecule comprising the nucleic acid SEQ ID NO. 7947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 7947 or polypeptide SEQ ID NO. 7948, respectively, or a homolog or a fragment thereof, is increased or generated, or if the activity b1522-protein is increased or generated in a non-human organism, like a microorganism or a plant cell, plant or part thereof, especially with non-targeted localization, whereby the respective line disclose in column 7 the fine chemical coenzyme Q10. For example, an increase of the coenzyme Q10 of at least 1 percent, particularly in a range of 56 to 198-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a b1522-protein, or if the activity of the polypeptide B1522, preferably represented by SEQ ID NO. 7948, or a homolog or fragment thereof, or if the activity of a polypeptide encoded by a nucleic acid molecule comprising the nucleic acid SEQ ID NO. 7947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 7947 or polypeptide SEQ ID NO. 7948, respectively, or a homolog or a fragment thereof, is increased or generated, or if the activity b1522-protein is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 36 to 144-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a acid shock protein, or if the activity of the polypeptide B1597, preferably represented by SEQ ID NO. 38301, or a homolog or fragment thereof, or if the activity of a polypeptide encoded by a nucleic acid molecule comprising the nucleic acid SEQ ID NO. 38300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 38300 or polypeptide SEQ ID NO. 38301, respectively, or a homolog or a fragment thereof, is increased or generated, or if the activity acid shock protein is increased or generated in a non-human organism, like a microorganism or a plant cell, plant or part thereof, especially with non-targeted localization, whereby the respective line disclose in column 7 the fine chemical coenzyme Q10. For example, an increase of the coenzyme Q10 of at least 1 percent, particularly in a range of 81 to 159-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a acid shock protein, or if the activity of the polypeptide B1597, preferably represented by SEQ ID NO. 38301, or a homolog or fragment thereof, or if the activity of a polypeptide encoded by a nucleic acid molecule comprising the nucleic acid SEQ ID NO. 38300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 38300 or polypeptide SEQ ID NO. 38301, respectively, or a homolog or a fragment thereof, is increased or generated, or if the activity acid shock protein is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 46 to 168-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a serine/threonine-protein phosphatase, or if the activity of the polypeptide B1838, preferably represented by SEQ ID NO. 8317, or a homolog or fragment thereof, or if the activity of a polypeptide encoded by a nucleic acid molecule comprising the nucleic acid SEQ ID NO. 8316, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 8316 or polypeptide SEQ ID NO. 8317, respectively, or a homolog or a fragment thereof, is increased or generated, or if the activity serine/threonine-protein phosphatase is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 36 to 94-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a b2032-protein, or if the activity of the polypeptide B2032, preferably represented by SEQ ID NO. 8921, or a homolog or fragment thereof, or if the activity of a polypeptide encoded by a nucleic acid molecule comprising the nucleic acid SEQ ID NO. 8920, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 8920 or polypeptide SEQ ID NO. 8921, respectively, or a homolog or a fragment thereof, is increased or generated, or if the activity b2032-protein is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 36 to 118-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a colanic acid biosynthesis protein, or if the activity of the polypeptide B2043, preferably represented by SEQ ID NO. 112194, or a homolog or fragment thereof, or if the activity of a polypeptide encoded by a nucleic acid molecule comprising the nucleic acid SEQ ID NO. 112193, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 112193 or polypeptide SEQ ID NO. 112194, respectively, or a homolog or a fragment thereof, is increased or generated, or if the activity colanic acid biosynthesis protein is increased or generated in a non-human organism, like a microorganism or a plant cell, plant or part thereof, especially with non-targeted localization, whereby the respective line disclose in column 7 the fine chemical coenzyme Q10. For example, an increase of the coenzyme Q10 of at least 1 percent, particularly in a range of 128 to 257-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a NADH dehydrogenase I chain I, or if the activity of the polypeptide B2281, preferably represented by SEQ ID NO. 39121, or a homolog or fragment thereof, or if the activity of a polypeptide encoded by a nucleic acid molecule comprising the nucleic acid SEQ ID NO. 39120, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 39120 or polypeptide SEQ ID NO. 39121, respectively, or a homolog or a fragment thereof, is increased or generated, or if the activity NADH dehydrogenase I chain I is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 19 to 33-percent is conferred as compared to a corresponding non-transformed wild type non-human organism.

Accordingly, in one embodiment, an increase of nicotinic acid in a non-human organism, as compared to a corresponding non-transformed wild type non-human organism, is conferred in the process of the invention, if the activity of a polypeptide showing the activity of a ATP synthase subunit E, or if the activity of the polypeptide B2285, preferably represented by SEQ ID NO. 99621, or a homolog or fragment thereof, or if the activity of a polypeptide encoded by a nucleic acid molecule comprising the nucleic acid SEQ ID NO. 99620, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 99620 or polypeptide SEQ ID NO. 99621, respectively, or a homolog or a fragment thereof, is increased or generated, or if the activity ATP synthase subunit E is increased or generated in a non-human organism, like a microorganism or a plant cell, plant or part thereof, especially with non-targeted localization, whereby the respective line disclose in column 7 the fine chemical nicotinic acid. For example, an increase of the nicotinic acid of at least 1 percent, particularly in a range of 26 to 42-percent is conferred as compared to a corresponding non-transformed wild type non-human organism.

Accordingly, in one embodiment, an increase of nicotinic acid in a non-human organism, as compared to a corresponding non-transformed wild type non-human organism, is conferred in the process of the invention, if the activity of a polypeptide showing the activity of a L-aspartate oxidase, or if the activity of the polypeptide B2574, preferably represented by SEQ ID NO. 138378, or a homolog or fragment thereof, or if the activity of a polypeptide encoded by a nucleic acid molecule comprising the nucleic acid SEQ ID NO. 138377, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 138377 or polypeptide SEQ ID NO. 138378, respectively, or a homolog or a fragment thereof, is increased or generated, or if the activity L-aspartate oxidase is increased or generated in a non-human organism, like a microorganism or a plant cell, plant or part thereof, especially with plastidic localization, whereby the respective line disclose in column 7 the fine chemical nicotinic acid. For example, an increase of the nicotinic acid of at least 1 percent, particularly in a range of 24 to 430-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a b2739-protein, or if the activity of the polypeptide B2739, preferably represented by SEQ ID NO. 80907, or a homolog or fragment thereof, or if the activity of a polypeptide encoded by a nucleic acid molecule comprising the nucleic acid SEQ ID NO. 80906, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 80906 or polypeptide SEQ ID NO. 80907, respectively, or a homolog or a fragment thereof, is increased or generated, or if the activity b2739-protein is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 37 to 114-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a 2-C-methyl-D-erythritol 4-phosphate cytidylyltransferase, or if the activity of the polypeptide B2747, preferably represented by SEQ ID NO. 81076, or a homolog or fragment thereof, or if the activity of a polypeptide encoded by a nucleic acid molecule comprising the nucleic acid SEQ ID NO. 81075, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 81075 or polypeptide SEQ ID NO. 81076, respectively, or a homolog or a fragment thereof, is increased or generated, or if the activity 2-C-methyl-D-erythritol 4-phosphate cytidylyltransferase is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 70 to 125-percent is conferred as compared to a corresponding non-transformed wild type non-human organism.

Accordingly, in one embodiment, an increase of lipoic acid in a non-human organism, as compared to a corresponding non-transformed wild type non-human organism, is conferred in the process of the invention, if the activity of a polypeptide showing the activity of a phosphoadenosine phosphosulfate reductase, or if the activity of the polypeptide B2762, preferably represented by SEQ ID NO. 81452, or a homolog or fragment thereof, or if the activity of a polypeptide encoded by a nucleic acid molecule comprising the nucleic acid SEQ ID NO. 81451, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 81451 or polypeptide SEQ ID NO. 81452, respectively, or a homolog or a fragment thereof, is increased or generated, or if the activity phosphoadenosine phosphosulfate reductase is increased or generated in a non-human organism, like a microorganism or a plant cell, plant or part thereof, especially with non-targeted localization, whereby the respective line disclose in column 7 the fine chemical lipoic acid. For example, an increase of the lipoic acid of at least 1 percent, particularly in a range of 472 to 18201-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a b2849-protein, or if the activity of the polypeptide B2849, preferably represented by SEQ ID NO. 81981, or a homolog or fragment thereof, or if the activity of a polypeptide encoded by a nucleic acid molecule comprising the nucleic acid SEQ ID NO. 81980, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 81980 or polypeptide SEQ ID NO. 81981, respectively, or a homolog or a fragment thereof, is increased or generated, or if the activity b2849-protein is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 38 to 73-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a methyltransferase, or if the activity of the polypeptide B3262, preferably represented by SEQ ID NO. 42503, or a homolog or fragment thereof, or if the activity of a polypeptide encoded by a nucleic acid molecule comprising the nucleic acid SEQ ID NO. 42502, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 42502 or polypeptide SEQ ID NO. 42503, respectively, or a homolog or a fragment thereof, is increased or generated, or if the activity methyltransferase is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 49 to 107-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a Ribose import ATP-binding protein, or if the activity of the polypeptide B3749, preferably represented by SEQ ID NO. 138771, or a homolog or fragment thereof, or if the activity of a polypeptide encoded by a nucleic acid molecule comprising the nucleic acid SEQ ID NO. 138770, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 138770 or polypeptide SEQ ID NO. 138771, respectively, or a homolog or a fragment thereof, is increased or generated, or if the activity Ribose import ATP-binding protein is increased or generated in a non-human organism, like a microorganism or a plant cell, plant or part thereof, especially with non-targeted localization, whereby the respective line disclose in column 7 the fine chemical coenzyme Q10. For example, an increase of the coenzyme Q10 of at least 1 percent, particularly in a range of 74 to 169-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a b3817-protein, or if the activity of the polypeptide B3817, preferably represented by SEQ ID NO. 10709, or a homolog or fragment thereof, or if the activity of a polypeptide encoded by a nucleic acid molecule comprising the nucleic acid SEQ ID NO. 10708, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 10708 or polypeptide SEQ ID NO. 10709, respectively, or a homolog or a fragment thereof, is increased or generated, or if the activity b3817-protein is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 84 to 98-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a glycerol dehydrogenase, or if the activity of the polypeptide B3945, preferably represented by SEQ ID NO. 44224, or a homolog or fragment thereof, or if the activity of a polypeptide encoded by a nucleic acid molecule comprising the nucleic acid SEQ ID NO. 44223, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 44223 or polypeptide SEQ ID NO. 44224, respectively, or a homolog or a fragment thereof, is increased or generated, or if the activity glycerol dehydrogenase is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 22 to 37-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a N-acetyl-gamma-glutamylphosphate reductase, or if the activity of the polypeptide B3958, preferably represented by SEQ ID NO. 138858, or a homolog or fragment thereof, or if the activity of a polypeptide encoded by a nucleic acid molecule comprising the nucleic acid SEQ ID NO. 138857, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 138857 or polypeptide SEQ ID NO. 138858, respectively, or a homolog or a fragment thereof, is increased or generated, or if the activity N-acetyl-gamma-glutamylphosphate reductase is increased or generated in a non-human organism, like a microorganism or a plant cell, plant or part thereof, especially with plastidic localization, whereby the respective line disclose in column 7 the fine chemical coenzyme Q10. For example, an increase of the coenzyme Q10 of at least 1 percent, particularly in a range of 24 to 32-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a b4029-protein, or if the activity of the polypeptide B4029, preferably represented by SEQ ID NO. 10741, or a homolog or fragment thereof, or if the activity of a polypeptide encoded by a nucleic acid molecule comprising the nucleic acid SEQ ID NO. 10740, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 10740 or polypeptide SEQ ID NO. 10741, respectively, or a homolog or a fragment thereof, is increased or generated, or if the activity b4029-protein is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 41 to 171-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a acetyltransferase, or if the activity of the polypeptide B4256, preferably represented by SEQ ID NO. 45322, or a homolog or fragment thereof, or if the activity of a polypeptide encoded by a nucleic acid molecule comprising the nucleic acid SEQ ID NO. 45321, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 45321 or polypeptide SEQ ID NO. 45322, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 43 to 135-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a gluconate transport system permease 3, or if the activity of the polypeptide B4321, preferably represented by SEQ ID NO. 45395, or a homolog or fragment thereof, or if the activity of a polypeptide encoded by a nucleic acid molecule comprising the nucleic acid SEQ ID NO. 45394, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 45394 or polypeptide SEQ ID NO. 45395, respectively, or a homolog or a fragment thereof, is increased or generated, or if the activity gluconate transport system permease 3 is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 47 to 110-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a transcription factor, or if the activity of the polypeptide GM02LC19289, preferably represented by SEQ ID NO. 46752, or a homolog or fragment thereof, or if the activity of a polypeptide encoded by a nucleic acid molecule comprising the nucleic acid SEQ ID NO. 46751, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 46751 or polypeptide SEQ ID NO. 46752, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical coenzyme Q10. For example, an increase of the coenzyme Q10 of at least 1 percent, particularly in a range of 61 to 107-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a transcription factor, or if the activity of the polypeptide GM02LC19289, preferably represented by SEQ ID NO. 46752, or a homolog or fragment thereof, or if the activity of a polypeptide encoded by a nucleic acid molecule comprising the nucleic acid SEQ ID NO. 46751, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 46751 or polypeptide SEQ ID NO. 46752, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 53 to 82-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a transcription factor, or if the activity of the polypeptide GM02LC21368, preferably represented by SEQ ID NO. 84199, or a homolog or fragment thereof, or if the activity of a polypeptide encoded by a nucleic acid molecule comprising the nucleic acid SEQ ID NO. 84198, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 84198 or polypeptide SEQ ID NO. 84199, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical coenzyme Q10. For example, an increase of the coenzyme Q10 of at least 1 percent, particularly in a range of 56 to 519-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a flavodoxin, or if the activity of the polypeptide Sll0248, preferably represented by SEQ ID NO. 49144, or a homolog or fragment thereof, or if the activity of a polypeptide encoded by a nucleic acid molecule comprising the nucleic acid SEQ ID NO. 49143, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 49143 or polypeptide SEQ ID NO. 49144, respectively, or a homolog or a fragment thereof, is increased or generated, or if the activity flavodoxin is increased or generated in a non-human organism, like a microorganism or a plant cell, plant or part thereof, especially with plastidic localization, whereby the respective line disclose in column 7 the fine chemical coenzyme Q10. For example, an increase of the coenzyme Q10 of at least 1 percent, particularly in a range of 95 to 573-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a uroporphyrin-III C-methyltransferase, or if the activity of the polypeptide Sll0378, preferably represented by SEQ ID NO. 84870, or a homolog or fragment thereof, or if the activity of a polypeptide encoded by a nucleic acid molecule comprising the nucleic acid SEQ ID NO. 84869, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 84869 or polypeptide SEQ ID NO. 84870, respectively, or a homolog or a fragment thereof, is increased or generated, or if the activity uroporphyrin-III C-methyltransferase is increased or generated in a non-human organism, like a microorganism or a plant cell, plant or part thereof, especially with plastidic localization, whereby the respective line disclose in column 7 the fine chemical coenzyme Q10. For example, an increase of the coenzyme Q10 of at least 1 percent, particularly in a range of 58 to 77-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a sll1761-protein, or if the activity of the polypeptide Sll1761, preferably represented by SEQ ID NO. 55380, or a homolog or fragment thereof, or if the activity of a polypeptide encoded by a nucleic acid molecule comprising the nucleic acid SEQ ID NO. 55379, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 55379 or polypeptide SEQ ID NO. 55380, respectively, or a homolog or a fragment thereof, is increased or generated, or if the activity sll1761-protein is increased or generated in a non-human organism, like a microorganism or a plant cell, plant or part thereof, especially with non-targeted localization, whereby the respective line disclose in column 7 the fine chemical coenzyme Q10. For example, an increase of the coenzyme Q10 of at least 1 percent, particularly in a range of 81 to 152-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a sll1761-protein, or if the activity of the polypeptide Sll1761, preferably represented by SEQ ID NO. 55380, or a homolog or fragment thereof, or if the activity of a polypeptide encoded by a nucleic acid molecule comprising the nucleic acid SEQ ID NO. 55379, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 55379 or polypeptide SEQ ID NO. 55380, respectively, or a homolog or a fragment thereof, is increased or generated, or if the activity sll1761-protein is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 48 to 91-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a cation-transporting ATPase, or if the activity of the polypeptide Sll1920, preferably represented by SEQ ID NO. 55386, or a homolog or fragment thereof, or if the activity of a polypeptide encoded by a nucleic acid molecule comprising the nucleic acid SEQ ID NO. 55385, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 55385 or polypeptide SEQ ID NO. 55386, respectively, or a homolog or a fragment thereof, is increased or generated, or if the activity cation-transporting ATPase is increased or generated in a non-human organism, like a microorganism or a plant cell, plant or part thereof, especially with mitochondrial localization, whereby the respective line disclose in column 7 the fine chemical coenzyme Q9. For example, an increase of the coenzyme Q9 of at least 1 percent, particularly in a range of 37 to 55-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a oxidoreductase, or if the activity of the polypeptide 51r0338, preferably represented by SEQ ID NO. 11991, or a homolog or fragment thereof, or if the activity of a polypeptide encoded by a nucleic acid molecule comprising the nucleic acid SEQ ID NO. 11990, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 11990 or polypeptide SEQ ID NO. 11991, respectively, or a homolog or a fragment thereof, is increased or generated, or if the activity oxidoreductase is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 47 to 62-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a malate dehydrogenase, or if the activity of the polypeptide 51r0721, preferably represented by SEQ ID NO. 87398, or a homolog or fragment thereof, or if the activity of a polypeptide encoded by a nucleic acid molecule comprising the nucleic acid SEQ ID NO. 87397, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 87397 or polypeptide SEQ ID NO. 87398, respectively, or a homolog or a fragment thereof, is increased or generated, or if the activity malate dehydrogenase is increased or generated in a non-human organism, like a microorganism or a plant cell, plant or part thereof, especially with plastidic localization, whereby the respective line disclose in column 7 the fine chemical coenzyme Q10. For example, an increase of the coenzyme Q10 of at least 1 percent, particularly in a range of 61 to 135-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a malate dehydrogenase, or if the activity of the polypeptide 51r0721, preferably represented by SEQ ID NO. 87398, or a homolog or fragment thereof, or if the activity of a polypeptide encoded by a nucleic acid molecule comprising the nucleic acid SEQ ID NO. 87397, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 87397 or polypeptide SEQ ID NO. 87398, respectively, or a homolog or a fragment thereof, is increased or generated, or if the activity malate dehydrogenase is increased or generated in a non-human organism, like a microorganism or a plant cell, plant or part thereof, especially with plastidic localization, whereby the respective line disclose in column 7 the fine chemical coenzyme Q9. For example, an increase of the coenzyme Q9 of at least 1 percent, particularly in a range of 56 to 88-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a permease protein of ABC transporter, or if the activity of the polypeptide Slr0949, preferably represented by SEQ ID NO. 119223, or a homolog or fragment thereof, or if the activity of a polypeptide encoded by a nucleic acid molecule comprising the nucleic acid SEQ ID NO. 119222, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 119222 or polypeptide SEQ ID NO. 119223, respectively, or a homolog or a fragment thereof, is increased or generated, or if the activity permease protein of ABC transporter is increased or generated in a non-human organism, like a microorganism or a plant cell, plant or part thereof, especially with mitochondrial localization, whereby the respective line disclose in column 7 the fine chemical coenzyme Q9. For example, an increase of the coenzyme Q9 of at least 1 percent, particularly in a range of 46 to 50-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a slr1107-protein, or if the activity of the polypeptide Slr1107, preferably represented by SEQ ID NO. 101777, or a homolog or fragment thereof, or if the activity of a polypeptide encoded by a nucleic acid molecule comprising the nucleic acid SEQ ID NO. 101776, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 101776 or polypeptide SEQ ID NO. 101777, respectively, or a homolog or a fragment thereof, is increased or generated, or if the activity slr1107-protein is increased or generated in a non-human organism, like a microorganism or a plant cell, plant or part thereof, especially with plastidic localization, whereby the respective line disclose in column 7 the fine chemical coenzyme Q9. For example, an increase of the coenzyme Q9 of at least 1 percent, particularly in a range of 39 to 135-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a fatty acid desaturase, or if the activity of the polypeptide Slr1350, preferably represented by SEQ ID NO. 116714, or a homolog or fragment thereof, or if the activity of a polypeptide encoded by a nucleic acid molecule comprising the nucleic acid SEQ ID NO. 116713, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 116713 or polypeptide SEQ ID NO. 116714, respectively, or a homolog or a fragment thereof, is increased or generated, or if the activity fatty acid desaturase is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 43 to 67-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a photosystem II protein, or if the activity of the polypeptide Slr1739, preferably represented by SEQ ID NO. 58732, or a homolog or fragment thereof, or if the activity of a polypeptide encoded by a nucleic acid molecule comprising the nucleic acid SEQ ID NO. 58731, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 58731 or polypeptide SEQ ID NO. 58732, respectively, or a homolog or a fragment thereof, is increased or generated, or if the activity photosystem II protein is increased or generated in a non-human organism, like a microorganism or a plant cell, plant or part thereof, especially with plastidic localization, whereby the respective line disclose in column 7 the fine chemical coenzyme Q9. For example, an increase of the coenzyme Q9 of at least 1 percent, particularly in a range of 58 to 64-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a glutamine amidotransferase, or if the activity of the polypeptide Slr1742, preferably represented by SEQ ID NO. 58752, or a homolog or fragment thereof, or if the activity of a polypeptide encoded by a nucleic acid molecule comprising the nucleic acid SEQ ID NO. 58751, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 58751 or polypeptide SEQ ID NO. 58752, respectively, or a homolog or a fragment thereof, is increased or generated, or if the activity glutamine amidotransferase is increased or generated in a non-human organism, like a microorganism or a plant cell, plant or part thereof, especially with mitochondrial localization, whereby the respective line disclose in column 7 the fine chemical coenzyme Q10. For example, an increase of the coenzyme Q10 of at least 1 percent, particularly in a range of 58 to 125-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a glycerol-3-phosphate dehydrogenase, or if the activity of the polypeptide Slr1755, preferably represented by SEQ ID NO. 58824, or a homolog or fragment thereof, or if the activity of a polypeptide encoded by a nucleic acid molecule comprising the nucleic acid SEQ ID NO. 58823, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 58823 or polypeptide SEQ ID NO. 58824, respectively, or a homolog or a fragment thereof, is increased or generated, or if the activity glycerol-3-phosphate dehydrogenase is increased or generated in a non-human organism, like a microorganism or a plant cell, plant or part thereof, especially with plastidic localization, whereby the respective line disclose in column 7 the fine chemical coenzyme Q10. For example, an increase of the coenzyme Q10 of at least 1 percent, particularly in a range of 53 to 133-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a glycerol-3-phosphate dehydrogenase, or if the activity of the polypeptide Slr1755, preferably represented by SEQ ID NO. 58824, or a homolog or fragment thereof, or if the activity of a polypeptide encoded by a nucleic acid molecule comprising the nucleic acid SEQ ID NO. 58823, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 58823 or polypeptide SEQ ID NO. 58824, respectively, or a homolog or a fragment thereof, is increased or generated, or if the activity glycerol-3-phosphate dehydrogenase is increased or generated in a non-human organism, like a microorganism or a plant cell, plant or part thereof, especially with plastidic localization, whereby the respective line disclose in column 7 the fine chemical coenzyme Q9. For example, an increase of the coenzyme Q9 of at least 1 percent, particularly in a range of 49 to 132-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a Sec-independent protein translocase subunit, or if the activity of the polypeptide TTC0019, preferably represented by SEQ ID NO. 12699, or a homolog or fragment thereof, or if the activity of a polypeptide encoded by a nucleic acid molecule comprising the nucleic acid SEQ ID NO. 12698, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 12698 or polypeptide SEQ ID NO. 12699, respectively, or a homolog or a fragment thereof, is increased or generated, or if the activity Sec-independent protein translocase subunit is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 36 to 141-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a amino acid ABC transporter permease protein, or if the activity of the polypeptide TTCO337, preferably represented by SEQ ID NO. 61071, or a homolog or fragment thereof, or if the activity of a polypeptide encoded by a nucleic acid molecule comprising the nucleic acid SEQ ID NO. 61070, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 61070 or polypeptide SEQ ID NO. 61071, respectively, or a homolog or a fragment thereof, is increased or generated, or if the activity amino acid ABC transporter permease protein is increased or generated in a non-human organism, like a microorganism or a plant cell, plant or part thereof, especially with non-targeted localization, whereby the respective line disclose in column 7 the fine chemical coenzyme Q10. For example, an increase of the coenzyme Q10 of at least 1 percent, particularly in a range of 64 to 181-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a multiple antibiotic resistance protein, or if the activity of the polypeptide TTC1193, preferably represented by SEQ ID NO. 61724, or a homolog or fragment thereof, or if the activity of a polypeptide encoded by a nucleic acid molecule comprising the nucleic acid SEQ ID NO. 61723, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 61723 or polypeptide SEQ ID NO. 61724, respectively, or a homolog or a fragment thereof, is increased or generated, or if the activity multiple antibiotic resistance protein is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 45 to 91-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a TTC1386-protein, or if the activity of the polypeptide TTC1386, preferably represented by SEQ ID NO. 62080, or a homolog or fragment thereof, or if the activity of a polypeptide encoded by a nucleic acid molecule comprising the nucleic acid SEQ ID NO. 62079, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 62079 or polypeptide SEQ ID NO. 62080, respectively, or a homolog or a fragment thereof, is increased or generated, or if the activity TTC1386-protein is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 37 to 147-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a cell division control protein, or if the activity of the polypeptide Ybr160w, preferably represented by SEQ ID NO. 62718, or a homolog or fragment thereof, or if the activity of a polypeptide encoded by a nucleic acid molecule comprising the nucleic acid SEQ ID NO. 62717, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 62717 or polypeptide SEQ ID NO. 62718, respectively, or a homolog or a fragment thereof, is increased or generated, or if the activity cell division control protein is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 70 to 78-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a ycl027w-protein, or if the activity of the polypeptide Ycl027w, preferably represented by SEQ ID NO. 96981, or a homolog or fragment thereof, or if the activity of a polypeptide encoded by a nucleic acid molecule comprising the nucleic acid SEQ ID NO. 96980, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 96980 or polypeptide SEQ ID NO. 96981, respectively, or a homolog or a fragment thereof, is increased or generated, or if the activity ycl027w-protein is increased or generated in a non-human organism, like a microorganism or a plant cell, plant or part thereof, especially with plastidic localization, whereby the respective line disclose in column 7 the fine chemical coenzyme Q10. For example, an increase of the coenzyme Q10 of at least 1 percent, particularly in a range of 96 to 120-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a protein kinase, or if the activity of the polypeptide Ydl159w, preferably represented by SEQ ID NO. 14276, or a homolog or fragment thereof, or if the activity of a polypeptide encoded by a nucleic acid molecule comprising the nucleic acid SEQ ID NO. 14275, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 14275 or polypeptide SEQ ID NO. 14276, respectively, or a homolog or a fragment thereof, is increased or generated, or if the activity protein kinase is increased or generated in a non-human organism, like a microorganism or a plant cell, plant or part thereof, especially with plastidic localization, whereby the respective line disclose in column 7 the fine chemical coenzyme Q9. For example, an increase of the coenzyme Q9 of at least 1 percent, particularly in a range of 41 to 74-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a halotolerance protein, or if the activity of the polypeptide Ykr072c, preferably represented by SEQ ID NO. 130904, or a homolog or fragment thereof, or if the activity of a polypeptide encoded by a nucleic acid molecule comprising the nucleic acid SEQ ID NO. 130903, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 130903 or polypeptide SEQ ID NO. 130904, respectively, or a homolog or a fragment thereof, is increased or generated, or if the activity halotolerance protein is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 38 to 91-percent is conferred as compared to a corresponding non-transformed wild type non-human organism.

Accordingly, in one embodiment, an increase of nicotinic acid in a non-human organism, as compared to a corresponding non-transformed wild type non-human organism, is conferred in the process of the invention, if the activity of a polypeptide showing the activity of a lipase, or if the activity of the polypeptide Ykr089c, preferably represented by SEQ ID NO. 102062, or a homolog or fragment thereof, or if the activity of a polypeptide encoded by a nucleic acid molecule comprising the nucleic acid SEQ ID NO. 102061, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 102061 or polypeptide SEQ ID NO. 102062, respectively, or a homolog or a fragment thereof, is increased or generated, or if the activity lipase is increased or generated in a non-human organism, like a microorganism or a plant cell, plant or part thereof, especially with non-targeted localization, whereby the respective line disclose in column 7 the fine chemical nicotinic acid. For example, an increase of the nicotinic acid of at least 1 percent, particularly in a range of 45 to 52-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a heat shock protein Hsp40, or if the activity of the polypeptide Ynl007c, preferably represented by SEQ ID NO. 139174, or a homolog or fragment thereof, or if the activity of a polypeptide encoded by a nucleic acid molecule comprising the nucleic acid SEQ ID NO. 139173, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 139173 or polypeptide SEQ ID NO. 139174, respectively, or a homolog or a fragment thereof, is increased or generated, or if the activity heat shock protein Hsp40 is increased or generated in a non-human organism, like a microorganism or a plant cell, plant or part thereof, especially with non-targeted localization, whereby the respective line disclose in column 7 the fine chemical coenzyme Q10. For example, an increase of the coenzyme Q10 of at least 1 percent, particularly in a range of 51 to 78-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a heat shock protein Hsp40, or if the activity of the polypeptide Ynl007c, preferably represented by SEQ ID NO. 139174, or a homolog or fragment thereof, or if the activity of a polypeptide encoded by a nucleic acid molecule comprising the nucleic acid SEQ ID NO. 139173, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 139173 or polypeptide SEQ ID NO. 139174, respectively, or a homolog or a fragment thereof, is increased or generated, or if the activity heat shock protein Hsp40 is increased or generated in a non-human organism, like a microorganism or a plant cell, plant or part thereof, especially with non-targeted localization, whereby the respective line disclose in column 7 the fine chemical coenzyme Q9. For example, an increase of the coenzyme Q9 of at least 1 percent, particularly in a range of 51 to 98-percent is conferred as compared to a corresponding non-transformed wild type non-human organism.

Accordingly, in one embodiment, an increase of coenzyme Q9 in a non-human organism, as compared to a corresponding non-transformed wild type non-human organism, is conferred in the process of the invention, if the activity of a polypeptide showing the activity of a histone acetyltransferase, or if the activity of the polypeptide Yor244w, preferably represented by SEQ ID NO. 92541, or a homolog or fragment thereof, or if the activity of a polypeptide encoded by a nucleic acid molecule comprising the nucleic acid SEQ ID NO. 92540, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 92540 or polypeptide SEQ ID NO. 92541, respectively, or a homolog or a fragment thereof, is increased or generated, or if the activity histone acetyltransferase is increased or generated in a non-human organism, like a microorganism or a plant cell, plant or part thereof, especially with plastidic localization, whereby the respective line disclose in column 7 the fine chemical coenzyme Q9. For example, an increase of the coenzyme Q9 of at least 1 percent, particularly in a range of 47 to 49-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a mitogenactivated protein kinase, or if the activity of the polypeptide Ypr054w, preferably represented by SEQ ID NO. 140111, or a homolog or fragment thereof, or if the activity of a polypeptide encoded by a nucleic acid molecule comprising the nucleic acid SEQ ID NO. 140110, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 140110 or polypeptide SEQ ID NO. 140111, respectively, or a homolog or a fragment thereof, is increased or generated, or if the activity mitogen-activated protein kinase is increased or generated in a non-human organism, like a microorganism or a plant cell, plant or part thereof, especially with non-targeted localization, whereby the respective line disclose in column 7 the fine chemical coenzyme Q10. For example, an increase of the coenzyme Q10 of at least 1 percent, particularly in a range of 53 to 250-percent is conferred as compared to a corresponding non-transformed wild type non-human organism.

Accordingly, in one embodiment, an increase of coenzyme Q10 in a non-human organism, as compared to a corresponding non-transformed wild type non-human organism, is conferred in the process of the invention, if the activity of a polypeptide showing the activity of a phosphopantothenoylcysteine decarboxylase, or if the activity of the polypeptide ZM06LC1143, preferably represented by SEQ ID NO. 68133, or a homolog or fragment thereof, or if the activity of a polypeptide encoded by a nucleic acid molecule comprising the nucleic acid SEQ ID NO. 68132, preferably the coding region thereof, or a homolog or fragment thereof, e.g. derived from Zea mays, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 16, column 5 or 8 in the respective same line as the nucleic acid molecule SEQ ID NO. 68132 or polypeptide SEQ ID NO. 68133, respectively, or a homolog or a fragment thereof, is increased or generated, or if the activity phosphopantothenoylcysteine decarboxylase is increased or generated in a non-human organism, like a microorganism or a plant cell, plant or part thereof, especially with non-targeted localization, whereby the respective line disclose in column 7 the fine chemical coenzyme Q10. For example, an increase of the coenzyme Q10 of at least 1 percent, particularly in a range of 81 to 678-percent is conferred as compared to a corresponding non-transformed wild type non-human organism.

[0096.1.7.16] to [0103.1.7.16] for the disclosure of these paragraphs see [0096.1.7.7] to [0103.1.7.7] above

In this paragraph “A” means “Goffeau et al., Science 274 (5287), 546 (1996)”, “B” means “Blattner et al., Science 277 (5331), 1453 (1997)”, “C” means “Nakamura et al., Nucleic Acids Res. 28(1), 72 (2000)”, “D” means “deposition of the sequences to the EMBL/GenBank/DDBJ databases under “Sequencing of the draft genome assembly of Azotobacter vinelandii AvOP”. in June 2005 by Copeland et al.”, “E” means “Henne et al., Nat. Biotechnol. 22 (5), 547 (2004)”, “F” means “Kaul et al., Nature 408 (6814), 796 (2000)”, and “G” means “Matsumoto et al., Nature 436 (7052), 793 (2005)”.

The nucleic acid sequence of AAC43185 from Arabidopsis thaliana, e.g. as shown in the respective line in column 5 of Table I, application no. 16, has been published in F. And the activity of the gene product thereof is the activity of amino acid acetyltransferase.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “amino acid acetyltransferase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said AAC43185, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said AAC43185, and preferably the activity is increased plastidic, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said AAC43185, or a functional equivalent or a homolog thereof as depicted in column 8 of Table II, application no. 16, preferably a homolog or functional equivalent as depicted in column 8 of Table II B, application no. 16, and being depicted in the same respective line as said AAC43185, and preferably the activity is increased plastidic, whereby the respective line disclose in column 7 the fine chemical coenzyme Q10.

Accordingly, in one embodiment, the molecule which activity is to be increased in the process of the invention is the gene product with an activity as a “amino acid acetyltransferase”, preferably it is the molecule of section (a) or (b) of this paragraph.

In particular, it was observed that in plants, especially in Arabidopsis thaliana, increasing or generating the activity of a gene product plastidic with the activity of a “amino acid acetyltransferase”, preferably being encoded by a gene comprising the nucleic acid sequence SEQ ID NO. 17356, preferably the coding region thereof, conferred the production of or the increase in coenzyme Q10 compared with the wild type control.

The nucleic acid sequence of At1g43850 from Arabidopsis thaliana, e.g. as shown in the respective line in column 5 of Table I, application no. 16, has been published in F. And the activity of the gene product thereof is the activity of transcriptional regulator.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “transcriptional regulator”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said At1g43850, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said At1g43850, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said At1g43850, or a functional equivalent or a homolog thereof as depicted in column 8 of Table II, application no. 16, preferably a homolog or functional equivalent as depicted in column 8 of Table II B, application no. 16, and being depicted in the same respective line as said At1g43850, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 18070, preferably the coding region thereof, conferred the production of or the increase in coenzyme Q9 compared with the wild type control.

The nucleic acid sequence of At1g47380 from Arabidopsis thaliana, e.g. as shown in the respective line in column 5 of Table I, application no. 16, has been published in F. And the activity of the gene product thereof is the activity of At1g47380-protein.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “At1g47380-protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said At1g47380, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said At1g47380, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said At1g47380, or a functional equivalent or a homolog thereof as depicted in column 8 of Table II, application no. 16, preferably a homolog or functional equivalent as depicted in column 8 of Table II B, application no. 16, and being depicted in the same respective line as said At1g47380, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “At1g47380-protein”, preferably it is the molecule of section (a) or (b) of this paragraph.

In particular, it was observed that in plants, especially in Arabidopsis thaliana, increasing or generating the activity of a gene product non-targeted with the activity of a “At1g47380-protein”, preferably being encoded by a gene comprising the nucleic acid sequence SEQ ID NO. 18122, preferably the coding region thereof, conferred the production of or the increase in coenzyme Q9 compared with the wild type control.

The nucleic acid sequence of At1g72770 from Arabidopsis thaliana, e.g. as shown in the respective line in column 5 of Table I, application no. 16, has been published in F. And the activity of the gene product thereof is the activity of protein phosphatase.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said At1g72770, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said At1g72770, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said At1g72770, or a functional equivalent or a homolog thereof as depicted in column 8 of Table II, application no. 16, preferably a homolog or functional equivalent as depicted in column 8 of Table II B, application no. 16, and being depicted in the same respective line as said At1g72770, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19419, preferably the coding region thereof, conferred the production of or the increase in coenzyme Q9 compared with the wild type control.

The nucleic acid sequence of At2g37040 from Arabidopsis thaliana, e.g. as shown in the respective line in column 5 of Table I, application no. 16, has been published in F. And the activity of the gene product thereof is the activity of phenylalanine ammonia-lyase.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “phenylalanine ammonia-lyase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said At2g37040, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said At2g37040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said At2g37040, or a functional equivalent or a homolog thereof as depicted in column 8 of Table II, application no. 16, preferably a homolog or functional equivalent as depicted in column 8 of Table II B, application no. 16, and being depicted in the same respective line as said At2g37040, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “phenylalanine ammonia-lyase”, preferably it is the molecule of section (a) or (b) of this paragraph.

In particular, it was observed that in plants, especially in Arabidopsis thaliana, increasing or generating the activity of a gene product non-targeted with the activity of a “phenylalanine ammonia-lyase”, preferably being encoded by a gene comprising the nucleic acid sequence SEQ ID NO. 133042, preferably the coding region thereof, conferred the production of or the increase in coenzyme Q9 compared with the wild type control.

The nucleic acid sequence of At2g42830 from Arabidopsis thaliana, e.g. as shown in the respective line in column 5 of Table I, application no. 16, has been published in F. And the activity of the gene product thereof is the activity of MADS box protein transcription factor.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “MADS box protein transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said At2g42830, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said At2g42830, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said At2g42830, or a functional equivalent or a homolog thereof as depicted in column 8 of Table II, application no. 16, preferably a homolog or functional equivalent as depicted in column 8 of Table II B, application no. 16, and being depicted in the same respective line as said At2g42830, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “MADS box protein transcription factor”, preferably it is the molecule of section (a) or (b) of this paragraph.

In particular, it was observed that in plants, especially in Arabidopsis thaliana, increasing or generating the activity of a gene product non-targeted with the activity of a “MADS box protein transcription factor”, preferably being encoded by a gene comprising the nucleic acid sequence SEQ ID NO. 108727, preferably the coding region thereof, conferred the production of or the increase in coenzyme Q9 compared with the wild type control.

The nucleic acid sequence of At2g47880 from Arabidopsis thaliana, e.g. as shown in the respective line in column 5 of Table I, application no. 16, has been published in F. And the activity of the gene product thereof is the activity of glutaredoxin.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said At2g47880, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said At2g47880, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said At2g47880, or a functional equivalent or a homolog thereof as depicted in column 8 of Table II, application no. 16, preferably a homolog or functional equivalent as depicted in column 8 of Table II B, application no. 16, and being depicted in the same respective line as said At2g47880, and preferably the activity is increased non-targeted, whereby the respective line disclose in column 7 the fine chemical coenzyme Q10.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 21159, preferably the coding region thereof, conferred the production of or the increase in coenzyme Q10 compared with the wild type control.

The nucleic acid sequence of At3g02990 from Arabidopsis thaliana, e.g. as shown in the respective line in column 5 of Table I, application no. 16, has been published in F. And the activity of the gene product thereof is the activity of heat shock transcription factor.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said At3g02990, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said At3g02990, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said At3g02990, or a functional equivalent or a homolog thereof as depicted in column 8 of Table II, application no. 16, preferably a homolog or functional equivalent as depicted in column 8 of Table II B, application no. 16, and being depicted in the same respective line as said At3g02990, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 68657, preferably the coding region thereof, conferred the production of or the increase in coenzyme Q9 compared with the wild type control.

The nucleic acid sequence of At3g04050 from Arabidopsis thaliana, e.g. as shown in the respective line in column 5 of Table I, application no. 16, has been published in F. And the activity of the gene product thereof is the activity of pyruvate kinase.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “pyruvate kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said At3g04050, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said At3g04050, and preferably the activity is increased plastidic, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said At3g04050, or a functional equivalent or a homolog thereof as depicted in column 8 of Table II, application no. 16, preferably a homolog or functional equivalent as depicted in column 8 of Table II B, application no. 16, and being depicted in the same respective line as said At3g04050, and preferably the activity is increased plastidic, whereby the respective line disclose in column 7 the fine chemical coenzyme Q10.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 21497, preferably the coding region thereof, conferred the production of or the increase in coenzyme Q10 compared with the wild type control.

The nucleic acid sequence of At3g16820 from Arabidopsis thaliana, e.g. as shown in the respective line in column 5 of Table I, application no. 16, has been published in F. And the activity of the gene product thereof is the activity of F-box protein.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “F-box protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said At3g16820, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said At3g16820, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said At3g16820, or a functional equivalent or a homolog thereof as depicted in column 8 of Table II, application no. 16, preferably a homolog or functional equivalent as depicted in column 8 of Table II B, application no. 16, and being depicted in the same respective line as said At3g16820, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “F-box protein”, preferably it is the molecule of section (a) or (b) of this paragraph.

In particular, it was observed that in plants, especially in Arabidopsis thaliana, increasing or generating the activity of a gene product non-targeted with the activity of a “F-box protein”, preferably being encoded by a gene comprising the nucleic acid sequence SEQ ID NO. 120952, preferably the coding region thereof, conferred the production of or the increase in coenzyme Q9 compared with the wild type control.

The nucleic acid sequence of At3g20910 from Arabidopsis thaliana, e.g. as shown in the respective line in column 5 of Table I, application no. 16, has been published in F. And the activity of the gene product thereof is the activity of CCAAT-binding transcription factor.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “CCAAT-binding transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said At3g20910, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said At3g20910, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said At3g20910, or a functional equivalent or a homolog thereof as depicted in column 8 of Table II, application no. 16, preferably a homolog or functional equivalent as depicted in column 8 of Table II B, application no. 16, and being depicted in the same respective line as said At3g20910, and preferably the activity is increased non-targeted, whereby the respective line disclose in column 7 the fine chemical coenzyme Q10.

Accordingly, in one embodiment, the molecule which activity is to be increased in the process of the invention is the gene product with an activity as a “CCAAT-binding transcription factor”, preferably it is the molecule of section (a) or (b) of this paragraph.

In particular, it was observed that in plants, especially in Arabidopsis thaliana, increasing or generating the activity of a gene product non-targeted with the activity of a “CCAAT-binding transcription factor”, preferably being encoded by a gene comprising the nucleic acid sequence SEQ ID NO. 22921, preferably the coding region thereof, conferred the production of or the increase in coenzyme Q10 compared with the wild type control.

The nucleic acid sequence of At3g20910 from Arabidopsis thaliana, e.g. as shown in the respective line in column 5 of Table I, application no. 16, has been published in F. And the activity of the gene product thereof is the activity of CCAAT-binding transcription factor.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “CCAAT-binding transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said At3g20910, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said At3g20910, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said At3g20910, or a functional equivalent or a homolog thereof as depicted in column 8 of Table II, application no. 16, preferably a homolog or functional equivalent as depicted in column 8 of Table II B, application no. 16, and being depicted in the same respective line as said At3g20910, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “CCAAT-binding transcription factor”, preferably it is the molecule of section (a) or (b) of this paragraph.

In particular, it was observed that in plants, especially in Arabidopsis thaliana, increasing or generating the activity of a gene product non-targeted with the activity of a “CCAAT-binding transcription factor”, preferably being encoded by a gene comprising the nucleic acid sequence SEQ ID NO. 22921, preferably the coding region thereof, conferred the production of or the increase in coenzyme Q9 compared with the wild type control.

The nucleic acid sequence of At3g27540 from Arabidopsis thaliana, e.g. as shown in the respective line in column 5 of Table I, application no. 16, has been published in F. And the activity of the gene product thereof is the activity of glycosyl transferase.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “glycosyl transferase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said At3g27540, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said At3g27540, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said At3g27540, or a functional equivalent or a homolog thereof as depicted in column 8 of Table II, application no. 16, preferably a homolog or functional equivalent as depicted in column 8 of Table II B, application no. 16, and being depicted in the same respective line as said At3g27540, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “glycosyl transferase”, preferably it is the molecule of section (a) or (b) of this paragraph.

In particular, it was observed that in plants, especially in Arabidopsis thaliana, increasing or generating the activity of a gene product non-targeted with the activity of a “glycosyl transferase”, preferably being encoded by a gene comprising the nucleic acid sequence SEQ ID NO. 68777, preferably the coding region thereof, conferred the production of or the increase in coenzyme Q9 compared with the wild type control.

The nucleic acid sequence of At3g61830 from Arabidopsis thaliana, e.g. as shown in the respective line in column 5 of Table I, application no. 16, has been published in F. And the activity of the gene product thereof is the activity of auxin response factor.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “auxin response factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said At3g61830, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said At3g61830, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said At3g61830, or a functional equivalent or a homolog thereof as depicted in column 8 of Table II, application no. 16, preferably a homolog or functional equivalent as depicted in column 8 of Table II B, application no. 16, and being depicted in the same respective line as said At3g61830, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “auxin response factor”, preferably it is the molecule of section (a) or (b) of this paragraph.

In particular, it was observed that in plants, especially in Arabidopsis thaliana, increasing or generating the activity of a gene product non-targeted with the activity of a “auxin response factor”, preferably being encoded by a gene comprising the nucleic acid sequence SEQ ID NO. 2367, preferably the coding region thereof, conferred the production of or the increase in coenzyme Q9 compared with the wild type control.

The nucleic acid sequence of At3g62950 from Arabidopsis thaliana, e.g. as shown in the respective line in column 5 of Table I, application no. 16, has been published in F. And the activity of the gene product thereof is the activity of glutaredoxin.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said At3g62950, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said At3g62950, or a functional equivalent or a homolog thereof as depicted in column 8 of Table II, application no. 16, preferably a homolog or functional equivalent as depicted in column 8 of Table II B, application no. 16, and being depicted in the same respective line as said At3g62950, and preferably the activity is increased non-targeted, whereby the respective line disclose in column 7 the fine chemical coenzyme Q10.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in coenzyme Q10 compared with the wild type control.

The nucleic acid sequence of At3g62950 from Arabidopsis thaliana, e.g. as shown in the respective line in column 5 of Table I, application no. 16, has been published in F. And the activity of the gene product thereof is the activity of glutaredoxin.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said At3g62950, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said At3g62950, or a functional equivalent or a homolog thereof as depicted in column 8 of Table II, application no. 16, preferably a homolog or functional equivalent as depicted in column 8 of Table II B, application no. 16, and being depicted in the same respective line as said At3g62950, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in coenzyme Q9 compared with the wild type control.

The nucleic acid sequence of At4g12110 from Arabidopsis thaliana, e.g. as shown in the respective line in column 5 of Table I, application no. 16, has been published in F. And the activity of the gene product thereof is the activity of sterol desaturase.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “sterol desaturase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said At4g12110, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said At4g12110, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said At4g12110, or a functional equivalent or a homolog thereof as depicted in column 8 of Table II, application no. 16, preferably a homolog or functional equivalent as depicted in column 8 of Table II B, application no. 16, and being depicted in the same respective line as said At4g12110, and preferably the activity is increased non-targeted, whereby the respective line disclose in column 7 the fine chemical coenzyme Q10.

Accordingly, in one embodiment, the molecule which activity is to be increased in the process of the invention is the gene product with an activity as a “sterol desaturase”, preferably it is the molecule of section (a) or (b) of this paragraph.

In particular, it was observed that in plants, especially in Arabidopsis thaliana, increasing or generating the activity of a gene product non-targeted with the activity of a “sterol desaturase”, preferably being encoded by a gene comprising the nucleic acid sequence SEQ ID NO. 131208, preferably the coding region thereof, conferred the production of or the increase in coenzyme Q10 compared with the wild type control.

The nucleic acid sequence of At4g15660 from Arabidopsis thaliana, e.g. as shown in the respective line in column 5 of Table I, application no. 16, has been published in F. And the activity of the gene product thereof is the activity of monothiol glutaredoxin.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said At4g15660, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said At4g15660, or a functional equivalent or a homolog thereof as depicted in column 8 of Table II, application no. 16, preferably a homolog or functional equivalent as depicted in column 8 of Table II B, application no. 16, and being depicted in the same respective line as said At4g15660, and preferably the activity is increased non-targeted, whereby the respective line disclose in column 7 the fine chemical coenzyme Q10.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in coenzyme Q10 compared with the wild type control.

The nucleic acid sequence of At4g15660 from Arabidopsis thaliana, e.g. as shown in the respective line in column 5 of Table I, application no. 16, has been published in F. And the activity of the gene product thereof is the activity of monothiol glutaredoxin.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said At4g15660, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said At4g15660, or a functional equivalent or a homolog thereof as depicted in column 8 of Table II, application no. 16, preferably a homolog or functional equivalent as depicted in column 8 of Table II B, application no. 16, and being depicted in the same respective line as said At4g15660, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in coenzyme Q9 compared with the wild type control.

The nucleic acid sequence of At4g34160 from Arabidopsis thaliana, e.g. as shown in the respective line in column 5 of Table I, application no. 16, has been published in F. And the activity of the gene product thereof is the activity of cyclin D.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “cyclin D”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said At4g34160, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said At4g34160, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said At4g34160, or a functional equivalent or a homolog thereof as depicted in column 8 of Table II, application no. 16, preferably a homolog or functional equivalent as depicted in column 8 of Table II B, application no. 16, and being depicted in the same respective line as said At4g34160, and preferably the activity is increased non-targeted, whereby the respective line disclose in column 7 the fine chemical coenzyme Q10.

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 24311, preferably the coding region thereof, conferred the production of or the increase in coenzyme Q10 compared with the wild type control.

The nucleic acid sequence of At4g34160 from Arabidopsis thaliana, e.g. as shown in the respective line in column 5 of Table I, application no. 16, has been published in F. And the activity of the gene product thereof is the activity of cyclin D .

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “cyclin D”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said At4g34160, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said At4g34160, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said At4g34160, or a functional equivalent or a homolog thereof as depicted in column 8 of Table II, application no. 16, preferably a homolog or functional equivalent as depicted in column 8 of Table II B, application no. 16, and being depicted in the same respective line as said At4g34160, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 24311, preferably the coding region thereof, conferred the production of or the increase in coenzyme Q9 compared with the wild type control.

The nucleic acid sequence of At4g35310 from Arabidopsis thaliana, e.g. as shown in the respective line in column 5 of Table I, application no. 16, has been published in F. And the activity of the gene product thereof is the activity of calcium-dependent protein kinase.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said At4g35310, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said At4g35310, or a functional equivalent or a homolog thereof as depicted in column 8 of Table II, application no. 16, preferably a homolog or functional equivalent as depicted in column 8 of Table II B, application no. 16, and being depicted in the same respective line as said At4g35310, and preferably the activity is increased non-targeted, whereby the respective line disclose in column 7 the fine chemical coenzyme Q10.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in coenzyme Q10 compared with the wild type control.

The nucleic acid sequence of At4g35310 from Arabidopsis thaliana, e.g. as shown in the respective line in column 5 of Table I, application no. 16, has been published in F. And the activity of the gene product thereof is the activity of calcium-dependent protein kinase.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said At4g35310, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said At4g35310, or a functional equivalent or a homolog thereof as depicted in column 8 of Table II, application no. 16, preferably a homolog or functional equivalent as depicted in column 8 of Table II B, application no. 16, and being depicted in the same respective line as said At4g35310, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in coenzyme Q9 compared with the wild type control.

The nucleic acid sequence of At5g03720 from Arabidopsis thaliana, e.g. as shown in the respective line in column 5 of Table I, application no. 16, has been published in F. And the activity of the gene product thereof is the activity of heat shock transcription factor.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said At5g03720, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said At5g03720, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said At5g03720, or a functional equivalent or a homolog thereof as depicted in column 8 of Table II, application no. 16, preferably a homolog or functional equivalent as depicted in column 8 of Table II B, application no. 16, and being depicted in the same respective line as said At5g03720, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 24438, preferably the coding region thereof, conferred the production of or the increase in coenzyme Q9 compared with the wild type control.

The nucleic acid sequence of At5g18600 from Arabidopsis thaliana, e.g. as shown in the respective line in column 5 of Table I, application no. 16, has been published in F. And the activity of the gene product thereof is the activity of monothiol glutaredoxin.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said At5g18600, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said At5g18600, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said At5g18600, or a functional equivalent or a homolog thereof as depicted in column 8 of Table II, application no. 16, preferably a homolog or functional equivalent as depicted in column 8 of Table II B, application no. 16, and being depicted in the same respective line as said At5g18600, and preferably the activity is increased non-targeted, whereby the respective line disclose in column 7 the fine chemical coenzyme Q10.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 4904, preferably the coding region thereof, conferred the production of or the increase in coenzyme Q10 compared with the wild type control.

The nucleic acid sequence of At5g45060 from Arabidopsis thaliana, e.g. as shown in the respective line in column 5 of Table I, application no. 16, has been published in F. And the activity of the gene product thereof is the activity of disease resistance protein.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “disease resistance protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said At5g45060, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said At5g45060, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said At5g45060, or a functional equivalent or a homolog thereof as depicted in column 8 of Table II, application no. 16, preferably a homolog or functional equivalent as depicted in column 8 of Table II B, application no. 16, and being depicted in the same respective line as said At5g45060, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “disease resistance protein”, preferably it is the molecule of section (a) or (b) of this paragraph.

In particular, it was observed that in plants, especially in Arabidopsis thaliana, increasing or generating the activity of a gene product non-targeted with the activity of a “disease resistance protein”, preferably being encoded by a gene comprising the nucleic acid sequence SEQ ID NO. 102223, preferably the coding region thereof, conferred the production of or the increase in coenzyme Q9 compared with the wild type control.

The nucleic acid sequence of At5g59220 from Arabidopsis thaliana, e.g. as shown in the respective line in column 5 of Table I, application no. 16, has been published in F. And the activity of the gene product thereof is the activity of protein phosphatase.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said At5g59220, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said At5g59220, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said At5g59220, or a functional equivalent or a homolog thereof as depicted in column 8 of Table II, application no. 16, preferably a homolog or functional equivalent as depicted in column 8 of Table II B, application no. 16, and being depicted in the same respective line as said At5g59220, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 25498, preferably the coding region thereof, conferred the production of or the increase in coenzyme Q9 compared with the wild type control.

The nucleic acid sequence of At5g64920 from Arabidopsis thaliana, e.g. as shown in the respective line in column 5 of Table I, application no. 16, has been published in F. And the activity of the gene product thereof is the activity of zinc finger protein.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “zinc finger protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said At5g64920, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said At5g64920, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said At5g64920, or a functional equivalent or a homolog thereof as depicted in column 8 of Table II, application no. 16, preferably a homolog or functional equivalent as depicted in column 8 of Table II B, application no. 16, and being depicted in the same respective line as said At5g64920, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “zinc finger protein”, preferably it is the molecule of section (a) or (b) of this paragraph.

In particular, it was observed that in plants, especially in Arabidopsis thaliana, increasing or generating the activity of a gene product non-targeted with the activity of a “zinc finger protein”, preferably being encoded by a gene comprising the nucleic acid sequence SEQ ID NO. 5493, preferably the coding region thereof, conferred the production of or the increase in coenzyme Q9 compared with the wild type control.

The nucleic acid sequence of AvinDRAFT_{—}3209 from Azotobacter vinelandii, e.g. as shown in the respective line in column 5 of Table I, application no. 16, has been published in D. And the activity of the gene product thereof is the activity of transcriptional regulator.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “transcriptional regulator”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said AvinDRAFT_{—}3209, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said AvinDRAFT_{—}3209, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said AvinDRAFT_{—}3209, or a functional equivalent or a homolog thereof as depicted in column 8 of Table II, application no. 16, preferably a homolog or functional equivalent as depicted in column 8 of Table II B, application no. 16, and being depicted in the same respective line as said AvinDRAFT_{—}3209, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 29286, preferably the coding region thereof, conferred the production of or the increase in coenzyme Q9 compared with the wild type control.

The nucleic acid sequence of AvinDRAFT_{—}3605 from Azotobacter vinelandii, e.g. as shown in the respective line in column 5 of Table I, application no. 16, has been published in D. And the activity of the gene product thereof is the activity of CTP synthase.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “CTP synthase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said AvinDRAFT_{—}3605, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said AvinDRAFT_{—}3605, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said AvinDRAFT_{—}3605, or a functional equivalent or a homolog thereof as depicted in column 8 of Table II, application no. 16, preferably a homolog or functional equivalent as depicted in column 8 of Table II B, application no. 16, and being depicted in the same respective line as said AvinDRAFT_{—}3605, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “CTP synthase”, preferably it is the molecule of section (a) or (b) of this paragraph.

In particular, it was observed that in plants, especially in Arabidopsis thaliana, increasing or generating the activity of a gene product non-targeted with the activity of a “CTP synthase”, preferably being encoded by a gene comprising the nucleic acid sequence SEQ ID NO. 31026, preferably the coding region thereof, conferred the production of or the increase in coenzyme Q9 compared with the wild type control.

The nucleic acid sequence of AvinDRAFT_{—}3629 from Azotobacter vinelandii, e.g. as shown in the respective line in column 5 of Table I, application no. 16, has been published in D. And the activity of the gene product thereof is the activity of recombinase A.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “recombinase A”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said AvinDRAFT_{—}3629, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said AvinDRAFT_{—}3629, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said AvinDRAFT_{—}3629, or a functional equivalent or a homolog thereof as depicted in column 8 of Table II, application no. 16, preferably a homolog or functional equivalent as depicted in column 8 of Table II B, application no. 16, and being depicted in the same respective line as said AvinDRAFT_{—}3629, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “recombinase A”, preferably it is the molecule of section (a) or (b) of this paragraph.

In particular, it was observed that in plants, especially in Arabidopsis thaliana, increasing or generating the activity of a gene product non-targeted with the activity of a “recombinase A”, preferably being encoded by a gene comprising the nucleic acid sequence SEQ ID NO. 114231, preferably the coding region thereof, conferred the production of or the increase in coenzyme Q9 compared with the wild type control.

The nucleic acid sequence of AvinDRAFT_{—}4420 from Azotobacter vinelandii, e.g. as shown in the respective line in column 5 of Table I, application no. 16, has been published in D. And the activity of the gene product thereof is the activity of enoyl-CoA hydratase.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “enoyl-CoA hydratase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said AvinDRAFT_{—}4420, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said AvinDRAFT_{—}4420, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said AvinDRAFT_{—}4420, or a functional equivalent or a homolog thereof as depicted in column 8 of Table II, application no. 16, preferably a homolog or functional equivalent as depicted in column 8 of Table II B, application no. 16, and being depicted in the same respective line as said AvinDRAFT_{—}4420, and preferably the activity is increased non-targeted, whereby the respective line disclose in column 7 the fine chemical coenzyme Q10.

Accordingly, in one embodiment, the molecule which activity is to be increased in the process of the invention is the gene product with an activity as a “enoyl-CoA hydratase”, preferably it is the molecule of section (a) or (b) of this paragraph.

In particular, it was observed that in plants, especially in Arabidopsis thaliana, increasing or generating the activity of a gene product non-targeted with the activity of a “enoyl-CoA hydratase”, preferably being encoded by a gene comprising the nucleic acid sequence SEQ ID NO. 73038, preferably the coding region thereof, conferred the production of or the increase in coenzyme Q10 compared with the wild type control.

The nucleic acid sequence of AvinDRAFT_{—}4420 from Azotobacter vinelandii, e.g. as shown in the respective line in column 5 of Table I, application no. 16, has been published in D. And the activity of the gene product thereof is the activity of enoyl-CoA hydratase.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “enoyl-CoA hydratase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said AvinDRAFT_{—}4420, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said AvinDRAFT_{—}4420, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said AvinDRAFT_{—}4420, or a functional equivalent or a homolog thereof as depicted in column 8 of Table II, application no. 16, preferably a homolog or functional equivalent as depicted in column 8 of Table II B, application no. 16, and being depicted in the same respective line as said AvinDRAFT_{—}4420, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “enoyl-CoA hydratase”, preferably it is the molecule of section (a) or (b) of this paragraph.

In particular, it was observed that in plants, especially in Arabidopsis thaliana, increasing or generating the activity of a gene product non-targeted with the activity of a “enoyl-CoA hydratase”, preferably being encoded by a gene comprising the nucleic acid sequence SEQ ID NO. 73038, preferably the coding region thereof, conferred the production of or the increase in coenzyme Q9 compared with the wild type control.

The nucleic acid sequence of AvinDRAFT_{—}4847 from Azotobacter vinelandii, e.g. as shown in the respective line in column 5 of Table I, application no. 16, has been published in D. And the activity of the gene product thereof is the activity of isopropylmalate isomerase large subunit.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “isopropylmalate isomerase large subunit”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said AvinDRAFT_{—}4847, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said AvinDRAFT_{—}4847, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said AvinDRAFT_{—}4847, or a functional equivalent or a homolog thereof as depicted in column 8 of Table II, application no. 16, preferably a homolog or functional equivalent as depicted in column 8 of Table II B, application no. 16, and being depicted in the same respective line as said AvinDRAFT_{—}4847, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “isopropylmalate isomerase large subunit”, preferably it is the molecule of section (a) or (b) of this paragraph.

In particular, it was observed that in plants, especially in Arabidopsis thaliana, increasing or generating the activity of a gene product non-targeted with the activity of a “isopropylmalate isomerase large subunit”, preferably being encoded by a gene comprising the nucleic acid sequence SEQ ID NO. 102941, preferably the coding region thereof, conferred the production of or the increase in coenzyme Q9 compared with the wild type control.

The nucleic acid sequence of AvinDRAFT_{—}5103 from Azotobacter vinelandii, e.g. as shown in the respective line in column 5 of Table I, application no. 16, has been published in D. And the activity of the gene product thereof is the activity of hydrolase.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “hydrolase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said AvinDRAFT_{—}5103, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said AvinDRAFT_{—}5103, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said AvinDRAFT_{—}5103, or a functional equivalent or a homolog thereof as depicted in column 8 of Table II, application no. 16, preferably a homolog or functional equivalent as depicted in column 8 of Table II B, application no. 16, and being depicted in the same respective line as said AvinDRAFT_{—}5103, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “hydrolase”, preferably it is the molecule of section (a) or (b) of this paragraph.

In particular, it was observed that in plants, especially in Arabidopsis thaliana, increasing or generating the activity of a gene product non-targeted with the activity of a “hydrolase”, preferably being encoded by a gene comprising the nucleic acid sequence SEQ ID NO. 6510, preferably the coding region thereof, conferred the production of or the increase in coenzyme Q9 compared with the wild type control.

The nucleic acid sequence of AX653549 from Oryza sativa, e.g. as shown in the respective line in column 5 of Table I, application no. 16, has been published in G. And the activity of the gene product thereof is the activity of AX653549-protein.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “AX653549-protein”, especially from Oryza sativa or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said AX653549, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said AX653549, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said AX653549, or a functional equivalent or a homolog thereof as depicted in column 8 of Table II, application no. 16, preferably a homolog or functional equivalent as depicted in column 8 of Table II B, application no. 16, and being depicted in the same respective line as said AX653549, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “AX653549-protein”, preferably it is the molecule of section (a) or (b) of this paragraph.

In particular, it was observed that in plants, especially in Arabidopsis thaliana, increasing or generating the activity of a gene product non-targeted with the activity of a “AX653549-protein”, preferably being encoded by a gene comprising the nucleic acid sequence SEQ ID NO. 34301, preferably the coding region thereof, conferred the production of or the increase in coenzyme Q9 compared with the wild type control.

The nucleic acid sequence of AY087308 from Arabidopsis thaliana, e.g. as shown in the respective line in column 5 of Table I, application no. 16, has been published in F. And the activity of the gene product thereof is the activity of AY087308-protein.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “AY087308-protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said AY087308, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said AY087308, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said AY087308, or a functional equivalent or a homolog thereof as depicted in column 8 of Table II, application no. 16, preferably a homolog or functional equivalent as depicted in column 8 of Table II B, application no. 16, and being depicted in the same respective line as said AY087308, and preferably the activity is increased non-targeted, whereby the respective line disclose in column 7 the fine chemical coenzyme Q10.

Accordingly, in one embodiment, the molecule which activity is to be increased in the process of the invention is the gene product with an activity as a “AY087308-protein”, preferably it is the molecule of section (a) or (b) of this paragraph.

In particular, it was observed that in plants, especially in Arabidopsis thaliana, increasing or generating the activity of a gene product non-targeted with the activity of a “AY087308-protein”, preferably being encoded by a gene comprising the nucleic acid sequence SEQ ID NO. 34602, preferably the coding region thereof, conferred the production of or the increase in coenzyme Q10 compared with the wild type control.

The nucleic acid sequence of B0004 from Escherichia coli, e.g. as shown in the respective line in column 5 of Table I, application no. 16, has been published in B. And the activity of the gene product thereof is the activity of threonine synthase.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “threonine synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said B0004, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B0004, and preferably the activity is increased plastidic, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B0004, or a functional equivalent or a homolog thereof as depicted in column 8 of Table II, application no. 16, preferably a homolog or functional equivalent as depicted in column 8 of Table II B, application no. 16, and being depicted in the same respective line as said B0004, and preferably the activity is increased plastidic, whereby the respective line disclose in column 7 the fine chemical coenzyme Q10.

Accordingly, in one embodiment, the molecule which activity is to be increased in the process of the invention is the gene product with an activity as a “threonine synthase”, preferably it is the molecule of section (a) or (b) of this paragraph.

In particular, it was observed that in plants, especially in Arabidopsis thaliana, increasing or generating the activity of a gene product plastidic with the activity of a “threonine synthase”, preferably being encoded by a gene comprising the nucleic acid sequence SEQ ID NO. 34889, preferably the coding region thereof, conferred the production of or the increase in coenzyme Q10 compared with the wild type control.

The nucleic acid sequence of B0077 from Escherichia coli, e.g. as shown in the respective line in column 5 of Table I, application no. 16, has been published in B. And the activity of the gene product thereof is the activity of acetolactate synthase III large subunit.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “acetolactate synthase III large subunit”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said B0077, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B0077, and preferably the activity is increased plastidic, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B0077, or a functional equivalent or a homolog thereof as depicted in column 8 of Table II, application no. 16, preferably a homolog or functional equivalent as depicted in column 8 of Table II B, application no. 16, and being depicted in the same respective line as said B0077, and preferably the activity is increased plastidic, whereby the respective line disclose in column 7 the fine chemical coenzyme Q10.

Accordingly, in one embodiment, the molecule which activity is to be increased in the process of the invention is the gene product with an activity as a “acetolactate synthase III large subunit”, preferably it is the molecule of section (a) or (b) of this paragraph.

In particular, it was observed that in plants, especially in Arabidopsis thaliana, increasing or generating the activity of a gene product plastidic with the activity of a “acetolactate synthase III large subunit”, preferably being encoded by a gene comprising the nucleic acid sequence SEQ ID NO. 137834, preferably the coding region thereof, conferred the production of or the increase in coenzyme Q10 compared with the wild type control.

The nucleic acid sequence of B0161 from Escherichia coli, e.g. as shown in the respective line in column 5 of Table I, application no. 16, has been published in B. And the activity of the gene product thereof is the activity of serine protease.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “serine protease”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said B0161, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B0161, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B0161, or a functional equivalent or a homolog thereof as depicted in column 8 of Table II, application no. 16, preferably a homolog or functional equivalent as depicted in column 8 of Table II B, application no. 16, and being depicted in the same respective line as said B0161, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “serine protease”, preferably it is the molecule of section (a) or (b) of this paragraph.

In particular, it was observed that in plants, especially in Arabidopsis thaliana, increasing or generating the activity of a gene product non-targeted with the activity of a “serine protease”, preferably being encoded by a gene comprising the nucleic acid sequence SEQ ID NO. 7081, preferably the coding region thereof, conferred the production of or the increase in coenzyme Q9 compared with the wild type control.

The nucleic acid sequence of B0344 from Escherichia coli, e.g. as shown in the respective line in column 5 of Table I, application no. 16, has been published in B. And the activity of the gene product thereof is the activity of beta-galactosidase.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “beta-galactosidase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said B0344, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B0344, and preferably the activity is increased plastidic, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B0344, or a functional equivalent or a homolog thereof as depicted in column 8 of Table II, application no. 16, preferably a homolog or functional equivalent as depicted in column 8 of Table II B, application no. 16, and being depicted in the same respective line as said B0344, and preferably the activity is increased plastidic, whereby the respective line disclose in column 7 the fine chemical coenzyme Q9.

Accordingly, in one embodiment, the molecule which activity is to be increased in the process of the invention is the gene product with an activity as a “beta-galactosidase”, preferably it is the molecule of section (a) or (b) of this paragraph.

In particular, it was observed that in plants, especially in Arabidopsis thaliana, increasing or generating the activity of a gene product plastidic with the activity of a “beta-galactosidase”, preferably being encoded by a gene comprising the nucleic acid sequence SEQ ID NO. 35733, preferably the coding region thereof, conferred the production of or the increase in coenzyme Q9 compared with the wild type control.

The nucleic acid sequence of B0449 from Escherichia coli, e.g. as shown in the respective line in column 5 of Table I, application no. 16, has been published in B. And the activity of the gene product thereof is the activity of ATP-binding component of a transport system.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “ATP-binding component of a transport system”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said B0449, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B0449, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B0449, or a functional equivalent or a homolog thereof as depicted in column 8 of Table II, application no. 16, preferably a homolog or functional equivalent as depicted in column 8 of Table II B, application no. 16, and being depicted in the same respective line as said B0449, and preferably the activity is increased non-targeted, whereby the respective line disclose in column 7 the fine chemical coenzyme Q10.

Accordingly, in one embodiment, the molecule which activity is to be increased in the process of the invention is the gene product with an activity as a “ATP-binding component of a transport system”, preferably it is the molecule of section (a) or (b) of this paragraph.

In particular, it was observed that in plants, especially in Arabidopsis thaliana, increasing or generating the activity of a gene product non-targeted with the activity of a “ATP-binding component of a transport system”, preferably being encoded by a gene comprising the nucleic acid sequence SEQ ID NO. 7333, preferably the coding region thereof, conferred the production of or the increase in coenzyme Q10 compared with the wild type control.

The nucleic acid sequence of B0449 from Escherichia coli, e.g. as shown in the respective line in column 5 of Table I, application no. 16, has been published in B. And the activity of the gene product thereof is the activity of ATP-binding component of a transport system.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “ATP-binding component of a transport system”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said B0449, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B0449, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B0449, or a functional equivalent or a homolog thereof as depicted in column 8 of Table II, application no. 16, preferably a homolog or functional equivalent as depicted in column 8 of Table II B, application no. 16, and being depicted in the same respective line as said B0449, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “ATP-binding component of a transport system”, preferably it is the molecule of section (a) or (b) of this paragraph.

In particular, it was observed that in plants, especially in Arabidopsis thaliana, increasing or generating the activity of a gene product non-targeted with the activity of a “ATP-binding component of a transport system”, preferably being encoded by a gene comprising the nucleic acid sequence SEQ ID NO. 7333, preferably the coding region thereof, conferred the production of or the increase in coenzyme Q9 compared with the wild type control.

The nucleic acid sequence of B0752 from Escherichia coli, e.g. as shown in the respective line in column 5 of Table I, application no. 16, has been published in B. And the activity of the gene product thereof is the activity of zinc transporter.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “zinc transporter”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said B0752, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B0752, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B0752, or a functional equivalent or a homolog thereof as depicted in column 8 of Table II, application no. 16, preferably a homolog or functional equivalent as depicted in column 8 of Table II B, application no. 16, and being depicted in the same respective line as said B0752, and preferably the activity is increased non-targeted, whereby the respective line disclose in column 7 the fine chemical coenzyme Q10.

Accordingly, in one embodiment, the molecule which activity is to be increased in the process of the invention is the gene product with an activity as a “zinc transporter”, preferably it is the molecule of section (a) or (b) of this paragraph.

In particular, it was observed that in plants, especially in Arabidopsis thaliana, increasing or generating the activity of a gene product non-targeted with the activity of a “zinc transporter”, preferably being encoded by a gene comprising the nucleic acid sequence SEQ ID NO. 36114, preferably the coding region thereof, conferred the production of or the increase in coenzyme Q10 compared with the wild type control.

The nucleic acid sequence of B0752 from Escherichia coli, e.g. as shown in the respective line in column 5 of Table I, application no. 16, has been published in B. And the activity of the gene product thereof is the activity of zinc transporter.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “zinc transporter”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said B0752, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B0752, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B0752, or a functional equivalent or a homolog thereof as depicted in column 8 of Table II, application no. 16, preferably a homolog or functional equivalent as depicted in column 8 of Table II B, application no. 16, and being depicted in the same respective line as said B0752, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “zinc transporter”, preferably it is the molecule of section (a) or (b) of this paragraph.

In particular, it was observed that in plants, especially in Arabidopsis thaliana, increasing or generating the activity of a gene product non-targeted with the activity of a “zinc transporter”, preferably being encoded by a gene comprising the nucleic acid sequence SEQ ID NO. 36114, preferably the coding region thereof, conferred the production of or the increase in coenzyme Q9 compared with the wild type control.

The nucleic acid sequence of B0963 from Escherichia coli, e.g. as shown in the respective line in column 5 of Table I, application no. 16, has been published in B. And the activity of the gene product thereof is the activity of methylglyoxal synthase.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “methylglyoxal synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said B0963, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B0963, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B0963, or a functional equivalent or a homolog thereof as depicted in column 8 of Table II, application no. 16, preferably a homolog or functional equivalent as depicted in column 8 of Table II B, application no. 16, and being depicted in the same respective line as said B0963, and preferably the activity is increased non-targeted, whereby the respective line disclose in column 7 the fine chemical coenzyme Q10.

Accordingly, in one embodiment, the molecule which activity is to be increased in the process of the invention is the gene product with an activity as a “methylglyoxal synthase”, preferably it is the molecule of section (a) or (b) of this paragraph.

In particular, it was observed that in plants, especially in Arabidopsis thaliana, increasing or generating the activity of a gene product non-targeted with the activity of a “methylglyoxal synthase”, preferably being encoded by a gene comprising the nucleic acid sequence SEQ ID NO. 36670, preferably the coding region thereof, conferred the production of or the increase in coenzyme Q10 compared with the wild type control.

The nucleic acid sequence of B0963 from Escherichia coli, e.g. as shown in the respective line in column 5 of Table I, application no. 16, has been published in B. And the activity of the gene product thereof is the activity of methylglyoxal synthase.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “methylglyoxal synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said B0963, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B0963, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B0963, or a functional equivalent or a homolog thereof as depicted in column 8 of Table II, application no. 16, preferably a homolog or functional equivalent as depicted in column 8 of Table II B, application no. 16, and being depicted in the same respective line as said B0963, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “methylglyoxal synthase”, preferably it is the molecule of section (a) or (b) of this paragraph.

In particular, it was observed that in plants, especially in Arabidopsis thaliana, increasing or generating the activity of a gene product non-targeted with the activity of a “methylglyoxal synthase”, preferably being encoded by a gene comprising the nucleic acid sequence SEQ ID NO. 36670, preferably the coding region thereof, conferred the production of or the increase in coenzyme Q9 compared with the wild type control.

The nucleic acid sequence of B1003 from Escherichia coli, e.g. as shown in the respective line in column 5 of Table I, application no. 16, has been published in B. And the activity of the gene product thereof is the activity of b1003-protein.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “b1003-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said B1003, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B1003, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B1003, or a functional equivalent or a homolog thereof as depicted in column 8 of Table II, application no. 16, preferably a homolog or functional equivalent as depicted in column 8 of Table II B, application no. 16, and being depicted in the same respective line as said B1003, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “b1003-protein”, preferably it is the molecule of section (a) or (b) of this paragraph.

In particular, it was observed that in plants, especially in Arabidopsis thaliana, increasing or generating the activity of a gene product non-targeted with the activity of a “b1003-protein”, preferably being encoded by a gene comprising the nucleic acid sequence SEQ ID NO. 7941, preferably the coding region thereof, conferred the production of or the increase in coenzyme Q9 compared with the wild type control.

The nucleic acid sequence of B1097 from Escherichia coli, e.g. as shown in the respective line in column 5 of Table I, application no. 16, has been published in B. And the activity of the gene product thereof is the activity of b1097-protein.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “b1097-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said B1097, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B1097, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B1097, or a functional equivalent or a homolog thereof as depicted in column 8 of Table II, application no. 16, preferably a homolog or functional equivalent as depicted in column 8 of Table II B, application no. 16, and being depicted in the same respective line as said B1097, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “b1097-protein”, preferably it is the molecule of section (a) or (b) of this paragraph.

In particular, it was observed that in plants, especially in Arabidopsis thaliana, increasing or generating the activity of a gene product non-targeted with the activity of a “b1097-protein”, preferably being encoded by a gene comprising the nucleic acid sequence SEQ ID NO. 138256, preferably the coding region thereof, conferred the production of or the increase in coenzyme Q9 compared with the wild type control.

The nucleic acid sequence of B1522 from Escherichia coli, e.g. as shown in the respective line in column 5 of Table I, application no. 16, has been published in B. And the activity of the gene product thereof is the activity of b1522-protein.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “b1522-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said B1522, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B1522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B1522, or a functional equivalent or a homolog thereof as depicted in column 8 of Table II, application no. 16, preferably a homolog or functional equivalent as depicted in column 8 of Table II B, application no. 16, and being depicted in the same respective line as said B1522, and preferably the activity is increased non-targeted, whereby the respective line disclose in column 7 the fine chemical coenzyme Q10.

Accordingly, in one embodiment, the molecule which activity is to be increased in the process of the invention is the gene product with an activity as a “b1522-protein”, preferably it is the molecule of section (a) or (b) of this paragraph.

In particular, it was observed that in plants, especially in Arabidopsis thaliana, increasing or generating the activity of a gene product non-targeted with the activity of a “b1522-protein”, preferably being encoded by a gene comprising the nucleic acid sequence SEQ ID NO. 7947, preferably the coding region thereof, conferred the production of or the increase in coenzyme Q10 compared with the wild type control.

The nucleic acid sequence of B1522 from Escherichia coli, e.g. as shown in the respective line in column 5 of Table I, application no. 16, has been published in B. And the activity of the gene product thereof is the activity of b1522-protein.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “b1522-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said B1522, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B1522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B1522, or a functional equivalent or a homolog thereof as depicted in column 8 of Table II, application no. 16, preferably a homolog or functional equivalent as depicted in column 8 of Table II B, application no. 16, and being depicted in the same respective line as said B1522, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “b1522-protein”, preferably it is the molecule of section (a) or (b) of this paragraph.

In particular, it was observed that in plants, especially in Arabidopsis thaliana, increasing or generating the activity of a gene product non-targeted with the activity of a “b1522-protein”, preferably being encoded by a gene comprising the nucleic acid sequence SEQ ID NO. 7947, preferably the coding region thereof, conferred the production of or the increase in coenzyme Q9 compared with the wild type control.

The nucleic acid sequence of B1597 from Escherichia coli, e.g. as shown in the respective line in column 5 of Table I, application no. 16, has been published in B. And the activity of the gene product thereof is the activity of acid shock protein.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “acid shock protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said B1597, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B1597, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B1597, or a functional equivalent or a homolog thereof as depicted in column 8 of Table II, application no. 16, preferably a homolog or functional equivalent as depicted in column 8 of Table II B, application no. 16, and being depicted in the same respective line as said B1597, and preferably the activity is increased non-targeted, whereby the respective line disclose in column 7 the fine chemical coenzyme Q10.

Accordingly, in one embodiment, the molecule which activity is to be increased in the process of the invention is the gene product with an activity as a “acid shock protein”, preferably it is the molecule of section (a) or (b) of this paragraph.

In particular, it was observed that in plants, especially in Arabidopsis thaliana, increasing or generating the activity of a gene product non-targeted with the activity of a “acid shock protein”, preferably being encoded by a gene comprising the nucleic acid sequence SEQ ID NO. 38300, preferably the coding region thereof, conferred the production of or the increase in coenzyme Q10 compared with the wild type control.

The nucleic acid sequence of B1597 from Escherichia coli, e.g. as shown in the respective line in column 5 of Table I, application no. 16, has been published in B. And the activity of the gene product thereof is the activity of acid shock protein.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “acid shock protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said B1597, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B1597, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B1597, or a functional equivalent or a homolog thereof as depicted in column 8 of Table II, application no. 16, preferably a homolog or functional equivalent as depicted in column 8 of Table II B, application no. 16, and being depicted in the same respective line as said B1597, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “acid shock protein”, preferably it is the molecule of section (a) or (b) of this paragraph.

In particular, it was observed that in plants, especially in Arabidopsis thaliana, increasing or generating the activity of a gene product non-targeted with the activity of a “acid shock protein”, preferably being encoded by a gene comprising the nucleic acid sequence SEQ ID NO. 38300, preferably the coding region thereof, conferred the production of or the increase in coenzyme Q9 compared with the wild type control.

The nucleic acid sequence of B1838 from Escherichia coli, e.g. as shown in the respective line in column 5 of Table I, application no. 16, has been published in B. And the activity of the gene product thereof is the activity of serine/threonine-protein phosphatase.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “serine/threonine-protein phosphatase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said B1838, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B1838, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B1838, or a functional equivalent or a homolog thereof as depicted in column 8 of Table II, application no. 16, preferably a homolog or functional equivalent as depicted in column 8 of Table II B, application no. 16, and being depicted in the same respective line as said B1838, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “serine/threonine-protein phosphatase”, preferably it is the molecule of section (a) or (b) of this paragraph.

In particular, it was observed that in plants, especially in Arabidopsis thaliana, increasing or generating the activity of a gene product non-targeted with the activity of a “serine/threonine-protein phosphatase”, preferably being encoded by a gene comprising the nucleic acid sequence SEQ ID NO. 8316, preferably the coding region thereof, conferred the production of or the increase in coenzyme Q9 compared with the wild type control.

The nucleic acid sequence of B2032 from Escherichia coli, e.g. as shown in the respective line in column 5 of Table I, application no. 16, has been published in B. And the activity of the gene product thereof is the activity of b2032-protein.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “b2032-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said B2032, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B2032, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B2032, or a functional equivalent or a homolog thereof as depicted in column 8 of Table II, application no. 16, preferably a homolog or functional equivalent as depicted in column 8 of Table II B, application no. 16, and being depicted in the same respective line as said B2032, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “b2032-protein”, preferably it is the molecule of section (a) or (b) of this paragraph.

In particular, it was observed that in plants, especially in Arabidopsis thaliana, increasing or generating the activity of a gene product non-targeted with the activity of a “b2032-protein”, preferably being encoded by a gene comprising the nucleic acid sequence SEQ ID NO. 8920, preferably the coding region thereof, conferred the production of or the increase in coenzyme Q9 compared with the wild type control.

The nucleic acid sequence of B2043 from Escherichia coli, e.g. as shown in the respective line in column 5 of Table I, application no. 16, has been published in B. And the activity of the gene product thereof is the activity of colanic acid biosynthesis protein.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “colanic acid biosynthesis protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said B2043, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B2043, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B2043, or a functional equivalent or a homolog thereof as depicted in column 8 of Table II, application no. 16, preferably a homolog or functional equivalent as depicted in column 8 of Table II B, application no. 16, and being depicted in the same respective line as said B2043, and preferably the activity is increased non-targeted, whereby the respective line disclose in column 7 the fine chemical coenzyme Q10.

Accordingly, in one embodiment, the molecule which activity is to be increased in the process of the invention is the gene product with an activity as a “colanic acid biosynthesis protein”, preferably it is the molecule of section (a) or (b) of this paragraph.

In particular, it was observed that in plants, especially in Arabidopsis thaliana, increasing or generating the activity of a gene product non-targeted with the activity of a “colanic acid biosynthesis protein”, preferably being encoded by a gene comprising the nucleic acid sequence SEQ ID NO. 112193, preferably the coding region thereof, conferred the production of or the increase in coenzyme Q10 compared with the wild type control.

The nucleic acid sequence of B2281 from Escherichia coli, e.g. as shown in the respective line in column 5 of Table I, application no. 16, has been published in B. And the activity of the gene product thereof is the activity of NADH dehydrogenase I chain I.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “NADH dehydrogenase I chain I”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said B2281, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B2281, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B2281, or a functional equivalent or a homolog thereof as depicted in column 8 of Table II, application no. 16, preferably a homolog or functional equivalent as depicted in column 8 of Table II B, application no. 16, and being depicted in the same respective line as said B2281, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “NADH dehydrogenase I chain I”, preferably it is the molecule of section (a) or (b) of this paragraph.

In particular, it was observed that in plants, especially in Arabidopsis thaliana, increasing or generating the activity of a gene product non-targeted with the activity of a “NADH dehydrogenase I chain I”, preferably being encoded by a gene comprising the nucleic acid sequence SEQ ID NO. 39120, preferably the coding region thereof, conferred the production of or the increase in coenzyme Q9 compared with the wild type control.

The nucleic acid sequence of B2285 from Escherichia coli, e.g. as shown in the respective line in column 5 of Table I, application no. 16, has been published in B. And the activity of the gene product thereof is the activity of ATP synthase subunit E.

Accordingly, in one embodiment, the process of the present invention for producing nicotinic acid in a non-human organism, like a microorganism or a plant or a part thereof, comprises increasing or generating the activity of a gene product with the activity of a gene product conferring the activity of “ATP synthase subunit E”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical nicotinic acid), application no. 16, preferably the coding region thereof, or a homolog or a fragment thereof, and being depicted in the same respective line as said B2285, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B2285, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B2285, or a functional equivalent or a homolog thereof as depicted in column 8 of Table II, application no. 16, preferably a homolog or functional equivalent as depicted in column 8 of Table II B, application no. 16, and being depicted in the same respective line as said B2285, and preferably the activity is increased non-targeted, whereby the respective line disclose in column 7 the fine chemical nicotinic acid.

Accordingly, in one embodiment, the molecule which activity is to be increased in the process of the invention is the gene product with an activity as a “ATP synthase subunit E”, preferably it is the molecule of section (a) or (b) of this paragraph.

In particular, it was observed that in plants, especially in Arabidopsis thaliana, increasing or generating the activity of a gene product non-targeted with the activity of a “ATP synthase subunit E”, preferably being encoded by a gene comprising the nucleic acid sequence SEQ ID NO. 99620, preferably the coding region thereof, conferred the production of or the increase in nicotinic acid compared with the wild type control.

The nucleic acid sequence of B2574 from Escherichia coli, e.g. as shown in the respective line in column 5 of Table I, application no. 16, has been published in B. And the activity of the gene product thereof is the activity of L-aspartate oxidase.

Accordingly, in one embodiment, the process of the present invention for producing nicotinic acid in a non-human organism, like a microorganism or a plant or a part thereof, comprises increasing or generating the activity of a gene product with the activity of a gene product conferring the activity of “L-aspartate oxidase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical nicotinic acid), application no. 16, preferably the coding region thereof, or a homolog or a fragment thereof, and being depicted in the same respective line as said B2574, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B2574, and preferably the activity is increased plastidic, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B2574, or a functional equivalent or a homolog thereof as depicted in column 8 of Table II, application no. 16, preferably a homolog or functional equivalent as depicted in column 8 of Table II B, application no. 16, and being depicted in the same respective line as said B2574, and preferably the activity is increased plastidic, whereby the respective line disclose in column 7 the fine chemical nicotinic acid.

Accordingly, in one embodiment, the molecule which activity is to be increased in the process of the invention is the gene product with an activity as a “L-aspartate oxidase”, preferably it is the molecule of section (a) or (b) of this paragraph.

In particular, it was observed that in plants, especially in Arabidopsis thaliana, increasing or generating the activity of a gene product plastidic with the activity of a “L-aspartate oxidase”, preferably being encoded by a gene comprising the nucleic acid sequence SEQ ID NO. 138377, preferably the coding region thereof, conferred the production of or the increase in nicotinic acid compared with the wild type control.

The nucleic acid sequence of B2739 from Escherichia coli, e.g. as shown in the respective line in column 5 of Table I, application no. 16, has been published in B. And the activity of the gene product thereof is the activity of b2739-protein.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “b2739-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said B2739, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B2739, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B2739, or a functional equivalent or a homolog thereof as depicted in column 8 of Table II, application no. 16, preferably a homolog or functional equivalent as depicted in column 8 of Table II B, application no. 16, and being depicted in the same respective line as said B2739, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “b2739-protein”, preferably it is the molecule of section (a) or (b) of this paragraph.

In particular, it was observed that in plants, especially in Arabidopsis thaliana, increasing or generating the activity of a gene product non-targeted with the activity of a “b2739-protein”, preferably being encoded by a gene comprising the nucleic acid sequence SEQ ID NO. 80906, preferably the coding region thereof, conferred the production of or the increase in coenzyme Q9 compared with the wild type control.

The nucleic acid sequence of B2747 from Escherichia coli, e.g. as shown in the respective line in column 5 of Table I, application no. 16, has been published in B. And the activity of the gene product thereof is the activity of 2-C-methyl-D-erythritol 4-phosphate cytidylyltransferase.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “2-C-methyl-D-erythritol 4-phosphate cytidylyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said B2747, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B2747, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B2747, or a functional equivalent or a homolog thereof as depicted in column 8 of Table II, application no. 16, preferably a homolog or functional equivalent as depicted in column 8 of Table II B, application no. 16, and being depicted in the same respective line as said B2747, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “2-C-methyl-D-erythritol 4-phosphate cytidylyltransferase”, preferably it is the molecule of section (a) or (b) of this paragraph.

In particular, it was observed that in plants, especially in Arabidopsis thaliana, increasing or generating the activity of a gene product non-targeted with the activity of a “2-C-methyl-D-erythritol 4-phosphate cytidylyltransferase”, preferably being encoded by a gene comprising the nucleic acid sequence SEQ ID NO. 81075, preferably the coding region thereof, conferred the production of or the increase in coenzyme Q9 compared with the wild type control.

The nucleic acid sequence of B2762 from Escherichia coli, e.g. as shown in the respective line in column 5 of Table I, application no. 16, has been published in B. And the activity of the gene product thereof is the activity of phosphoadenosine phosphosulfate reductase.

Accordingly, in one embodiment, the process of the present invention for producing lipoic acid in a non-human organism, like a microorganism or a plant or a part thereof, comprises increasing or generating the activity of a gene product with the activity of a gene product conferring the activity of “phosphoadenosine phosphosulfate reductase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical lipoic acid), application no. 16, preferably the coding region thereof, or a homolog or a fragment thereof, and being depicted in the same respective line as said B2762, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B2762, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B2762, or a functional equivalent or a homolog thereof as depicted in column 8 of Table II, application no. 16, preferably a homolog or functional equivalent as depicted in column 8 of Table II B, application no. 16, and being depicted in the same respective line as said B2762, and preferably the activity is increased non-targeted, whereby the respective line disclose in column 7 the fine chemical lipoic acid.

Accordingly, in one embodiment, the molecule which activity is to be increased in the process of the invention is the gene product with an activity as a “phosphoadenosine phosphosulfate reductase”, preferably it is the molecule of section (a) or (b) of this paragraph.

In particular, it was observed that in plants, especially in Arabidopsis thaliana, increasing or generating the activity of a gene product non-targeted with the activity of a “phosphoadenosine phosphosulfate reductase”, preferably being encoded by a gene comprising the nucleic acid sequence SEQ ID NO. 81451, preferably the coding region thereof, conferred the production of or the increase in lipoic acid compared with the wild type control.

The nucleic acid sequence of B2849 from Escherichia coli, e.g. as shown in the respective line in column 5 of Table I, application no. 16, has been published in B. And the activity of the gene product thereof is the activity of b2849-protein.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “b2849-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said B2849, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B2849, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B2849, or a functional equivalent or a homolog thereof as depicted in column 8 of Table II, application no. 16, preferably a homolog or functional equivalent as depicted in column 8 of Table II B, application no. 16, and being depicted in the same respective line as said B2849, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “b2849-protein”, preferably it is the molecule of section (a) or (b) of this paragraph.

In particular, it was observed that in plants, especially in Arabidopsis thaliana, increasing or generating the activity of a gene product non-targeted with the activity of a “b2849-protein”, preferably being encoded by a gene comprising the nucleic acid sequence SEQ ID NO. 81980, preferably the coding region thereof, conferred the production of or the increase in coenzyme Q9 compared with the wild type control.

The nucleic acid sequence of B3262 from Escherichia coli, e.g. as shown in the respective line in column 5 of Table I, application no. 16, has been published in B. And the activity of the gene product thereof is the activity of methyltransferase.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “methyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said B3262, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B3262, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B3262, or a functional equivalent or a homolog thereof as depicted in column 8 of Table II, application no. 16, preferably a homolog or functional equivalent as depicted in column 8 of Table II B, application no. 16, and being depicted in the same respective line as said B3262, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “methyltransferase”, preferably it is the molecule of section (a) or (b) of this paragraph.

In particular, it was observed that in plants, especially in Arabidopsis thaliana, increasing or generating the activity of a gene product non-targeted with the activity of a “methyltransferase”, preferably being encoded by a gene comprising the nucleic acid sequence SEQ ID NO. 42502, preferably the coding region thereof, conferred the production of or the increase in coenzyme Q9 compared with the wild type control.

The nucleic acid sequence of B3749 from Escherichia coli, e.g. as shown in the respective line in column 5 of Table I, application no. 16, has been published in B. And the activity of the gene product thereof is the activity of Ribose import ATP-binding protein.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “Ribose import ATP-binding protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said B3749, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B3749, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B3749, or a functional equivalent or a homolog thereof as depicted in column 8 of Table II, application no. 16, preferably a homolog or functional equivalent as depicted in column 8 of Table II B, application no. 16, and being depicted in the same respective line as said B3749, and preferably the activity is increased non-targeted, whereby the respective line disclose in column 7 the fine chemical coenzyme Q10.

Accordingly, in one embodiment, the molecule which activity is to be increased in the process of the invention is the gene product with an activity as a “Ribose import ATP-binding protein”, preferably it is the molecule of section (a) or (b) of this paragraph.

In particular, it was observed that in plants, especially in Arabidopsis thaliana, increasing or generating the activity of a gene product non-targeted with the activity of a “Ribose import ATP-binding protein”, preferably being encoded by a gene comprising the nucleic acid sequence SEQ ID NO. 138770, preferably the coding region thereof, conferred the production of or the increase in coenzyme Q10 compared with the wild type control.

The nucleic acid sequence of B3817 from Escherichia coli, e.g. as shown in the respective line in column 5 of Table I, application no. 16, has been published in B. And the activity of the gene product thereof is the activity of b3817-protein.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “b3817-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said B3817, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B3817, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B3817, or a functional equivalent or a homolog thereof as depicted in column 8 of Table II, application no. 16, preferably a homolog or functional equivalent as depicted in column 8 of Table II B, application no. 16, and being depicted in the same respective line as said B3817, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “b3817-protein”, preferably it is the molecule of section (a) or (b) of this paragraph.

In particular, it was observed that in plants, especially in Arabidopsis thaliana, increasing or generating the activity of a gene product non-targeted with the activity of a “b3817-protein”, preferably being encoded by a gene comprising the nucleic acid sequence SEQ ID NO. 10708, preferably the coding region thereof, conferred the production of or the increase in coenzyme Q9 compared with the wild type control.

The nucleic acid sequence of B3945 from Escherichia coli, e.g. as shown in the respective line in column 5 of Table I, application no. 16, has been published in B. And the activity of the gene product thereof is the activity of glycerol dehydrogenase.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “glycerol dehydrogenase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said B3945, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B3945, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B3945, or a functional equivalent or a homolog thereof as depicted in column 8 of Table II, application no. 16, preferably a homolog or functional equivalent as depicted in column 8 of Table II B, application no. 16, and being depicted in the same respective line as said B3945, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “glycerol dehydrogenase”, preferably it is the molecule of section (a) or (b) of this paragraph.

In particular, it was observed that in plants, especially in Arabidopsis thaliana, increasing or generating the activity of a gene product non-targeted with the activity of a “glycerol dehydrogenase”, preferably being encoded by a gene comprising the nucleic acid sequence SEQ ID NO. 44223, preferably the coding region thereof, conferred the production of or the increase in coenzyme Q9 compared with the wild type control.

The nucleic acid sequence of B3958 from Escherichia coli, e.g. as shown in the respective line in column 5 of Table I, application no. 16, has been published in B. And the activity of the gene product thereof is the activity of N-acetyl-gamma-glutamylphosphate reductase.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “N-acetyl-gamma-glutamylphosphate reductase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said B3958, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B3958, and preferably the activity is increased plastidic, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B3958, or a functional equivalent or a homolog thereof as depicted in column 8 of Table II, application no. 16, preferably a homolog or functional equivalent as depicted in column 8 of Table II B, application no. 16, and being depicted in the same respective line as said B3958, and preferably the activity is increased plastidic, whereby the respective line disclose in column 7 the fine chemical coenzyme Q10.

Accordingly, in one embodiment, the molecule which activity is to be increased in the process of the invention is the gene product with an activity as a “N-acetyl-gamma-glutamylphosphate reductase”, preferably it is the molecule of section (a) or (b) of this paragraph.

In particular, it was observed that in plants, especially in Arabidopsis thaliana, increasing or generating the activity of a gene product plastidic with the activity of a “N-acetyl-gammaglutamylphosphate reductase”, preferably being encoded by a gene comprising the nucleic acid sequence SEQ ID NO. 138857, preferably the coding region thereof, conferred the production of or the increase in coenzyme Q10 compared with the wild type control.

The nucleic acid sequence of B4029 from Escherichia coli, e.g. as shown in the respective line in column 5 of Table I, application no. 16, has been published in B. And the activity of the gene product thereof is the activity of b4029-protein.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “b4029-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said B4029, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B4029, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B4029, or a functional equivalent or a homolog thereof as depicted in column 8 of Table II, application no. 16, preferably a homolog or functional equivalent as depicted in column 8 of Table II B, application no. 16, and being depicted in the same respective line as said B4029, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “b4029-protein”, preferably it is the molecule of section (a) or (b) of this paragraph.

In particular, it was observed that in plants, especially in Arabidopsis thaliana, increasing or generating the activity of a gene product non-targeted with the activity of a “b4029-protein”, preferably being encoded by a gene comprising the nucleic acid sequence SEQ ID NO. 10740, preferably the coding region thereof, conferred the production of or the increase in coenzyme Q9 compared with the wild type control.

The nucleic acid sequence of B4256 from Escherichia coli, e.g. as shown in the respective line in column 5 of Table I, application no. 16, has been published in B. And the activity of the gene product thereof is the activity of acetyltransferase.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said B4256, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B4256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B4256, or a functional equivalent or a homolog thereof as depicted in column 8 of Table II, application no. 16, preferably a homolog or functional equivalent as depicted in column 8 of Table II B, application no. 16, and being depicted in the same respective line as said B4256, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 45321, preferably the coding region thereof, conferred the production of or the increase in coenzyme Q9 compared with the wild type control.

The nucleic acid sequence of B4321 from Escherichia coli, e.g. as shown in the respective line in column 5 of Table I, application no. 16, has been published in B. And the activity of the gene product thereof is the activity of gluconate transport system permease 3.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “gluconate transport system permease 3”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said B4321, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said B4321, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said B4321, or a functional equivalent or a homolog thereof as depicted in column 8 of Table II, application no. 16, preferably a homolog or functional equivalent as depicted in column 8 of Table II B, application no. 16, and being depicted in the same respective line as said B4321, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “gluconate transport system permease 3”, preferably it is the molecule of section (a) or (b) of this paragraph.

In particular, it was observed that in plants, especially in Arabidopsis thaliana, increasing or generating the activity of a gene product non-targeted with the activity of a “gluconate transport system permease 3”, preferably being encoded by a gene comprising the nucleic acid sequence SEQ ID NO. 45394, preferably the coding region thereof, conferred the production of or the increase in coenzyme Q9 compared with the wild type control.

The nucleic acid sequence of GM02LC19289 from Glycine max, e.g. as shown in the respective line in column 5 of Table I, application no. 16, is unpublished. And the activity of the gene product thereof is the activity of transcription factor.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said GM02LC19289, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said GM02LC19289, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said GM02LC19289, or a functional equivalent or a homolog thereof as depicted in column 8 of Table II, application no. 16, preferably a homolog or functional equivalent as depicted in column 8 of Table II B, application no. 16, and being depicted in the same respective line as said GM02LC19289, and preferably the activity is increased non-targeted, whereby the respective line disclose in column 7 the fine chemical coenzyme Q10.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 46751, preferably the coding region thereof, conferred the production of or the increase in coenzyme Q10 compared with the wild type control.

The nucleic acid sequence of GM02LC19289 from Glycine max, e.g. as shown in the respective line in column 5 of Table I, application no. 16, is unpublished. And the activity of the gene product thereof is the activity of transcription factor.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said

GM02LC19289, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said GM02LC19289, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said GM02LC19289, or a functional equivalent or a homolog thereof as depicted in column 8 of Table II, application no. 16, preferably a homolog or functional equivalent as depicted in column 8 of Table II B, application no. 16, and being depicted in the same respective line as said GM02LC19289, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 46751, preferably the coding region thereof, conferred the production of or the increase in coenzyme Q9 compared with the wild type control.

The nucleic acid sequence of GM02LC21368 from Glycine max, e.g. as shown in the respective line in column 5 of Table I, application no. 16, is unpublished. And the activity of the gene product thereof is the activity of transcription factor.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said GM02LC21368, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said GM02LC21368, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said GM02LC21368, or a functional equivalent or a homolog thereof as depicted in column 8 of Table II, application no. 16, preferably a homolog or functional equivalent as depicted in column 8 of Table II B, application no. 16, and being depicted in the same respective line as said GM02LC21368, and preferably the activity is increased non-targeted, whereby the respective line disclose in column 7 the fine chemical coenzyme Q10.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 84198, preferably the coding region thereof, conferred the production of or the increase in coenzyme Q10 compared with the wild type control.

The nucleic acid sequence of Sll0248 from Synechocystis sp., e.g. as shown in the respective line in column 5 of Table I, application no. 16, has been published in C. And the activity of the gene product thereof is the activity of flavodoxin.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “flavodoxin”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said Sll0248, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said Sll0248, and preferably the activity is increased plastidic, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said Sll0248, or a functional equivalent or a homolog thereof as depicted in column 8 of Table II, application no. 16, preferably a homolog or functional equivalent as depicted in column 8 of Table II B, application no. 16, and being depicted in the same respective line as said Sll0248, and preferably the activity is increased plastidic, whereby the respective line disclose in column 7 the fine chemical coenzyme Q10.

Accordingly, in one embodiment, the molecule which activity is to be increased in the process of the invention is the gene product with an activity as a “flavodoxin”, preferably it is the molecule of section (a) or (b) of this paragraph.

In particular, it was observed that in plants, especially in Arabidopsis thaliana, increasing or generating the activity of a gene product plastidic with the activity of a “flavodoxin”, preferably being encoded by a gene comprising the nucleic acid sequence SEQ ID NO. 49143, preferably the coding region thereof, conferred the production of or the increase in coenzyme Q10 compared with the wild type control.

The nucleic acid sequence of Sll0378 from Synechocystis sp., e.g. as shown in the respective line in column 5 of Table I, application no. 16, has been published in C. And the activity of the gene product thereof is the activity of uroporphyrin-III C-methyltransferase.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “uroporphyrin-III C-methyltransferase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said Sll0378, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said Sll0378, and preferably the activity is increased plastidic, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said Sll0378, or a functional equivalent or a homolog thereof as depicted in column 8 of Table II, application no. 16, preferably a homolog or functional equivalent as depicted in column 8 of Table II B, application no. 16, and being depicted in the same respective line as said Sll0378, and preferably the activity is increased plastidic, whereby the respective line disclose in column 7 the fine chemical coenzyme Q10.

Accordingly, in one embodiment, the molecule which activity is to be increased in the process of the invention is the gene product with an activity as a “uroporphyrin-III C-methyltransferase”, preferably it is the molecule of section (a) or (b) of this paragraph.

In particular, it was observed that in plants, especially in Arabidopsis thaliana, increasing or generating the activity of a gene product plastidic with the activity of a “uroporphyrin-III C-methyltransferase”, preferably being encoded by a gene comprising the nucleic acid sequence SEQ ID NO. 84869, preferably the coding region thereof, conferred the production of or the increase in coenzyme Q10 compared with the wild type control.

The nucleic acid sequence of Sll1761 from Synechocystis sp., e.g. as shown in the respective line in column 5 of Table I, application no. 16, has been published in C. And the activity of the gene product thereof is the activity of sll1761-protein.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “sll1761-protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said Sll1761, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said Sll1761, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said Sll1761, or a functional equivalent or a homolog thereof as depicted in column 8 of Table II, application no. 16, preferably a homolog or functional equivalent as depicted in column 8 of Table II B, application no. 16, and being depicted in the same respective line as said Sll1761, and preferably the activity is increased non-targeted, whereby the respective line disclose in column 7 the fine chemical coenzyme Q10.

Accordingly, in one embodiment, the molecule which activity is to be increased in the process of the invention is the gene product with an activity as a “sll1761-protein”, preferably it is the molecule of section (a) or (b) of this paragraph.

In particular, it was observed that in plants, especially in Arabidopsis thaliana, increasing or generating the activity of a gene product non-targeted with the activity of a “sll1761-protein”, preferably being encoded by a gene comprising the nucleic acid sequence SEQ ID NO. 55379, preferably the coding region thereof, conferred the production of or the increase in coenzyme Q10 compared with the wild type control.

The nucleic acid sequence of Sll1761 from Synechocystis sp., e.g. as shown in the respective line in column 5 of Table I, application no. 16, has been published in C. And the activity of the gene product thereof is the activity of sll1761-protein.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “sll1761-protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said Sll1761, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said Sll1761, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said Sll1761, or a functional equivalent or a homolog thereof as depicted in column 8 of Table II, application no. 16, preferably a homolog or functional equivalent as depicted in column 8 of Table II B, application no. 16, and being depicted in the same respective line as said Sll1761, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “sll1761-protein”, preferably it is the molecule of section (a) or (b) of this paragraph.

In particular, it was observed that in plants, especially in Arabidopsis thaliana, increasing or generating the activity of a gene product non-targeted with the activity of a “sll1761-protein”, preferably being encoded by a gene comprising the nucleic acid sequence SEQ ID NO. 55379, preferably the coding region thereof, conferred the production of or the increase in coenzyme Q9 compared with the wild type control.

The nucleic acid sequence of Sll1920 from Synechocystis sp., e.g. as shown in the respective line in column 5 of Table I, application no. 16, has been published in C. And the activity of the gene product thereof is the activity of cation-transporting ATPase.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “cation-transporting ATPase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said Sll1920, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said Sll1920, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said Sll1920, or a functional equivalent or a homolog thereof as depicted in column 8 of Table II, application no. 16, preferably a homolog or functional equivalent as depicted in column 8 of Table II B, application no. 16, and being depicted in the same respective line as said SI11920, and preferably the activity is increased mitochondrial, whereby the respective line disclose in column 7 the fine chemical coenzyme Q9.

Accordingly, in one embodiment, the molecule which activity is to be increased in the process of the invention is the gene product with an activity as a “cation-transporting ATPase”, preferably it is the molecule of section (a) or (b) of this paragraph.

In particular, it was observed that in plants, especially in Arabidopsis thaliana, increasing or generating the activity of a gene product mitochondrial with the activity of a “cation-transporting ATPase”, preferably being encoded by a gene comprising the nucleic acid sequence SEQ ID NO. 55385, preferably the coding region thereof, conferred the production of or the increase in coenzyme Q9 compared with the wild type control.

The nucleic acid sequence of Slr0338 from Synechocystis sp., e.g. as shown in the respective line in column 5 of Table I, application no. 16, has been published in C. And the activity of the gene product thereof is the activity of oxidoreductase.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “oxidoreductase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said Slr0338, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said Slr0338, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said Slr0338, or a functional equivalent or a homolog thereof as depicted in column 8 of Table II, application no. 16, preferably a homolog or functional equivalent as depicted in column 8 of Table II B, application no. 16, and being depicted in the same respective line as said Slr0338, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 11990, preferably the coding region thereof, conferred the production of or the increase in coenzyme Q9 compared with the wild type control.

The nucleic acid sequence of Slr0721 from Synechocystis sp., e.g. as shown in the respective line in column 5 of Table I, application no. 16, has been published in C. And the activity of the gene product thereof is the activity of malate dehydrogenase.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “malate dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said Slr0721, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said Slr0721, and preferably the activity is increased plastidic, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said Slr0721, or a functional equivalent or a homolog thereof as depicted in column 8 of Table II, application no. 16, preferably a homolog or functional equivalent as depicted in column 8 of Table II B, application no. 16, and being depicted in the same respective line as said Slr0721, and preferably the activity is increased plastidic, whereby the respective line disclose in column 7 the fine chemical coenzyme Q10.

Accordingly, in one embodiment, the molecule which activity is to be increased in the process of the invention is the gene product with an activity as a “malate dehydrogenase”, preferably it is the molecule of section (a) or (b) of this paragraph.

In particular, it was observed that in plants, especially in Arabidopsis thaliana, increasing or generating the activity of a gene product plastidic with the activity of a “malate dehydrogenase”, preferably being encoded by a gene comprising the nucleic acid sequence SEQ ID NO. 87397, preferably the coding region thereof, conferred the production of or the increase in coenzyme Q10 compared with the wild type control.

The nucleic acid sequence of Slr0721 from Synechocystis sp., e.g. as shown in the respective line in column 5 of Table I, application no. 16, has been published in C. And the activity of the gene product thereof is the activity of malate dehydrogenase.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “malate dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said Slr0721, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said Slr0721, and preferably the activity is increased plastidic, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said Slr0721, or a functional equivalent or a homolog thereof as depicted in column 8 of Table II, application no. 16, preferably a homolog or functional equivalent as depicted in column 8 of Table II B, application no. 16, and being depicted in the same respective line as said Slr0721, and preferably the activity is increased plastidic, whereby the respective line disclose in column 7 the fine chemical coenzyme Q9.

Accordingly, in one embodiment, the molecule which activity is to be increased in the process of the invention is the gene product with an activity as a “malate dehydrogenase”, preferably it is the molecule of section (a) or (b) of this paragraph.

In particular, it was observed that in plants, especially in Arabidopsis thaliana, increasing or generating the activity of a gene product plastidic with the activity of a “malate dehydrogenase”, preferably being encoded by a gene comprising the nucleic acid sequence SEQ ID NO. 87397, preferably the coding region thereof, conferred the production of or the increase in coenzyme Q9 compared with the wild type control.

The nucleic acid sequence of Slr0949 from Synechocystis sp., e.g. as shown in the respective line in column 5 of Table I, application no. 16, has been published in C. And the activity of the gene product thereof is the activity of permease protein of ABC transporter.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “permease protein of ABC transporter”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said Slr0949, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said Slr0949, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said Slr0949, or a functional equivalent or a homolog thereof as depicted in column 8 of Table II, application no. 16, preferably a homolog or functional equivalent as depicted in column 8 of Table II B, application no. 16, and being depicted in the same respective line as said Slr0949, and preferably the activity is increased mitochondrial, whereby the respective line disclose in column 7 the fine chemical coenzyme Q9.

Accordingly, in one embodiment, the molecule which activity is to be increased in the process of the invention is the gene product with an activity as a “permease protein of ABC transporter”, preferably it is the molecule of section (a) or (b) of this paragraph.

In particular, it was observed that in plants, especially in Arabidopsis thaliana, increasing or generating the activity of a gene product mitochondrial with the activity of a “permease protein of ABC transporter”, preferably being encoded by a gene comprising the nucleic acid sequence SEQ ID NO. 119222, preferably the coding region thereof, conferred the production of or the increase in coenzyme Q9 compared with the wild type control.

The nucleic acid sequence of Slr1107 from Synechocystis sp., e.g. as shown in the respective line in column 5 of Table I, application no. 16, has been published in C. And the activity of the gene product thereof is the activity of slr1107-protein.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “slr1107-protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said Slr1107, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said Slr1107, and preferably the activity is increased plastidic, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said Slr1107, or a functional equivalent or a homolog thereof as depicted in column 8 of Table II, application no. 16, preferably a homolog or functional equivalent as depicted in column 8 of Table II B, application no. 16, and being depicted in the same respective line as said Slr1107, and preferably the activity is increased plastidic, whereby the respective line disclose in column 7 the fine chemical coenzyme Q9.

Accordingly, in one embodiment, the molecule which activity is to be increased in the process of the invention is the gene product with an activity as a “slr1107-protein”, preferably it is the molecule of section (a) or (b) of this paragraph.

In particular, it was observed that in plants, especially in Arabidopsis thaliana, increasing or generating the activity of a gene product plastidic with the activity of a “slr1107-protein”, preferably being encoded by a gene comprising the nucleic acid sequence SEQ ID NO. 101776, preferably the coding region thereof, conferred the production of or the increase in coenzyme Q9 compared with the wild type control.

The nucleic acid sequence of Slr1350 from Synechocystis sp., e.g. as shown in the respective line in column 5 of Table I, application no. 16, has been published in C. And the activity of the gene product thereof is the activity of fatty acid desaturase.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “fatty acid desaturase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said Slr1350, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said Slr1350, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said Slr1350, or a functional equivalent or a homolog thereof as depicted in column 8 of Table II, application no. 16, preferably a homolog or functional equivalent as depicted in column 8 of Table II B, application no. 16, and being depicted in the same respective line as said Slr1350, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “fatty acid desaturase”, preferably it is the molecule of section (a) or (b) of this paragraph.

In particular, it was observed that in plants, especially in Arabidopsis thaliana, increasing or generating the activity of a gene product non-targeted with the activity of a “fatty acid desaturase”, preferably being encoded by a gene comprising the nucleic acid sequence SEQ ID NO. 116713, preferably the coding region thereof, conferred the production of or the increase in coenzyme Q9 compared with the wild type control.

The nucleic acid sequence of Slr1739 from Synechocystis sp., e.g. as shown in the respective line in column 5 of Table I, application no. 16, has been published in C. And the activity of the gene product thereof is the activity of photosystem II protein.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “photosystem II protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said Slr1739, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said Slr1739, and preferably the activity is increased plastidic, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said Slr1739, or a functional equivalent or a homolog thereof as depicted in column 8 of Table II, application no. 16, preferably a homolog or functional equivalent as depicted in column 8 of Table II B, application no. 16, and being depicted in the same respective line as said Slr1739, and preferably the activity is increased plastidic, whereby the respective line disclose in column 7 the fine chemical coenzyme Q9.

Accordingly, in one embodiment, the molecule which activity is to be increased in the process of the invention is the gene product with an activity as a “photosystem II protein”, preferably it is the molecule of section (a) or (b) of this paragraph.

In particular, it was observed that in plants, especially in Arabidopsis thaliana, increasing or generating the activity of a gene product plastidic with the activity of a “photosystem II protein”, preferably being encoded by a gene comprising the nucleic acid sequence SEQ ID NO. 58731, preferably the coding region thereof, conferred the production of or the increase in coenzyme Q9 compared with the wild type control.

The nucleic acid sequence of Slr1742 from Synechocystis sp., e.g. as shown in the respective line in column 5 of Table I, application no. 16, has been published in C. And the activity of the gene product thereof is the activity of glutamine amidotransferase.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “glutamine amidotransferase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said Slr1742, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said Slr1742, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said Slr1742, or a functional equivalent or a homolog thereof as depicted in column 8 of Table II, application no. 16, preferably a homolog or functional equivalent as depicted in column 8 of Table II B, application no. 16, and being depicted in the same respective line as said Slr1742, and preferably the activity is increased mitochondrial, whereby the respective line disclose in column 7 the fine chemical coenzyme Q10.

Accordingly, in one embodiment, the molecule which activity is to be increased in the process of the invention is the gene product with an activity as a “glutamine amidotransferase”, preferably it is the molecule of section (a) or (b) of this paragraph.

In particular, it was observed that in plants, especially in Arabidopsis thaliana, increasing or generating the activity of a gene product mitochondrial with the activity of a “glutamine amidotransferase”, preferably being encoded by a gene comprising the nucleic acid sequence SEQ ID NO. 58751, preferably the coding region thereof, conferred the production of or the increase in coenzyme Q10 compared with the wild type control.

The nucleic acid sequence of Slr1755 from Synechocystis sp., e.g. as shown in the respective line in column 5 of Table I, application no. 16, has been published in C. And the activity of the gene product thereof is the activity of glycerol-3-phosphate dehydrogenase.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “glycerol-3-phosphate dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said Slr1755, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said Slr1755, and preferably the activity is increased plastidic, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said Slr1755, or a functional equivalent or a homolog thereof as depicted in column 8 of Table II, application no. 16, preferably a homolog or functional equivalent as depicted in column 8 of Table II B, application no. 16, and being depicted in the same respective line as said Slr1755, and preferably the activity is increased plastidic, whereby the respective line disclose in column 7 the fine chemical coenzyme Q10.

Accordingly, in one embodiment, the molecule which activity is to be increased in the process of the invention is the gene product with an activity as a “glycerol-3-phosphate dehydrogenase”, preferably it is the molecule of section (a) or (b) of this paragraph.

In particular, it was observed that in plants, especially in Arabidopsis thaliana, increasing or generating the activity of a gene product plastidic with the activity of a “glycerol-3-phosphate dehydrogenase”, preferably being encoded by a gene comprising the nucleic acid sequence SEQ ID NO. 58823, preferably the coding region thereof, conferred the production of or the increase in coenzyme Q10 compared with the wild type control.

The nucleic acid sequence of Slr1755 from Synechocystis sp., e.g. as shown in the respective line in column 5 of Table I, application no. 16, has been published in C. And the activity of the gene product thereof is the activity of glycerol-3-phosphate dehydrogenase.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “glycerol-3-phosphate dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said Slr1755, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said Slr1755, and preferably the activity is increased plastidic, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said Slr1755, or a functional equivalent or a homolog thereof as depicted in column 8 of Table II, application no. 16, preferably a homolog or functional equivalent as depicted in column 8 of Table II B, application no. 16, and being depicted in the same respective line as said Slr1755, and preferably the activity is increased plastidic, whereby the respective line disclose in column 7 the fine chemical coenzyme Q9.

Accordingly, in one embodiment, the molecule which activity is to be increased in the process of the invention is the gene product with an activity as a “glycerol-3-phosphate dehydrogenase”, preferably it is the molecule of section (a) or (b) of this paragraph.

In particular, it was observed that in plants, especially in Arabidopsis thaliana, increasing or generating the activity of a gene product plastidic with the activity of a “glycerol-3-phosphate dehydrogenase”, preferably being encoded by a gene comprising the nucleic acid sequence SEQ ID NO. 58823, preferably the coding region thereof, conferred the production of or the increase in coenzyme Q9 compared with the wild type control.

The nucleic acid sequence of TTC0019 from Thermus thermophilus, e.g. as shown in the respective line in column 5 of Table I, application no. 16, has been published in E. And the activity of the gene product thereof is the activity of Sec-independent protein translocase subunit.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “Sec-independent protein translocase subunit”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said TTC0019, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said TTC0019, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said TTC0019, or a functional equivalent or a homolog thereof as depicted in column 8 of Table II, application no. 16, preferably a homolog or functional equivalent as depicted in column 8 of Table II B, application no. 16, and being depicted in the same respective line as said TTC0019, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “Sec-independent protein translocase subunit”, preferably it is the molecule of section (a) or (b) of this paragraph.

In particular, it was observed that in plants, especially in Arabidopsis thaliana, increasing or generating the activity of a gene product non-targeted with the activity of a “Sec-independent protein translocase subunit”, preferably being encoded by a gene comprising the nucleic acid sequence SEQ ID NO. 12698, preferably the coding region thereof, conferred the production of or the increase in coenzyme Q9 compared with the wild type control.

The nucleic acid sequence of TTC0337 from Thermus thermophilus, e.g. as shown in the respective line in column 5 of Table I, application no. 16, has been published in E. And the activity of the gene product thereof is the activity of amino acid ABC transporter permease protein.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “amino acid ABC transporter permease protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said TTC0337, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said TTC0337, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said TTC0337, or a functional equivalent or a homolog thereof as depicted in column 8 of Table II, application no. 16, preferably a homolog or functional equivalent as depicted in column 8 of Table II B, application no. 16, and being depicted in the same respective line as said TTC0337, and preferably the activity is increased non-targeted, whereby the respective line disclose in column 7 the fine chemical coenzyme Q10.

Accordingly, in one embodiment, the molecule which activity is to be increased in the process of the invention is the gene product with an activity as a “amino acid ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “amino acid ABC transporter permease protein”, preferably being encoded by a gene comprising the nucleic acid sequence SEQ ID NO. 61070, preferably the coding region thereof, conferred the production of or the increase in coenzyme Q10 compared with the wild type control.

The nucleic acid sequence of TTC1193 from Thermus thermophilus, e.g. as shown in the respective line in column 5 of Table I, application no. 16, has been published in E. And the activity of the gene product thereof is the activity of multiple antibiotic resistance protein.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “multiple antibiotic resistance protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said TTC1193, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said TTC1193, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said TTC1193, or a functional equivalent or a homolog thereof as depicted in column 8 of Table II, application no. 16, preferably a homolog or functional equivalent as depicted in column 8 of Table II B, application no. 16, and being depicted in the same respective line as said TTC1193, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “multiple antibiotic resistance protein”, preferably it is the molecule of section (a) or (b) of this paragraph.

In particular, it was observed that in plants, especially in Arabidopsis thaliana, increasing or generating the activity of a gene product non-targeted with the activity of a “multiple antibiotic resistance protein”, preferably being encoded by a gene comprising the nucleic acid sequence SEQ ID NO. 61723, preferably the coding region thereof, conferred the production of or the increase in coenzyme Q9 compared with the wild type control.

The nucleic acid sequence of TTC1386 from Thermus thermophilus, e.g. as shown in the respective line in column 5 of Table I, application no. 16, has been published in E. And the activity of the gene product thereof is the activity of TTC1386-protein.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “TTC1386-protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said TTC1386, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said TTC1386, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said TTC1386, or a functional equivalent or a homolog thereof as depicted in column 8 of Table II, application no. 16, preferably a homolog or functional equivalent as depicted in column 8 of Table II B, application no. 16, and being depicted in the same respective line as said TTC1386, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “TTC1386-protein”, preferably it is the molecule of section (a) or (b) of this paragraph.

In particular, it was observed that in plants, especially in Arabidopsis thaliana, increasing or generating the activity of a gene product non-targeted with the activity of a “TTC1386-protein”, preferably being encoded by a gene comprising the nucleic acid sequence SEQ ID NO. 62079, preferably the coding region thereof, conferred the production of or the increase in coenzyme Q9 compared with the wild type control.

The nucleic acid sequence of Ybr160w from Saccharomyces cerevisiae, e.g. as shown in the respective line in column 5 of Table I, application no. 16, has been published in A. And the activity of the gene product thereof is the activity of cell division control protein.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “cell division control protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said Ybr160w, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said Ybrl 60w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said Ybr160w, or a functional equivalent or a homolog thereof as depicted in column 8 of Table II, application no. 16, preferably a homolog or functional equivalent as depicted in column 8 of Table II B, application no. 16, and being depicted in the same respective line as said Ybr160w, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “cell division control protein”, preferably it is the molecule of section (a) or (b) of this paragraph.

In particular, it was observed that in plants, especially in Arabidopsis thaliana, increasing or generating the activity of a gene product non-targeted with the activity of a “cell division control protein”, preferably being encoded by a gene comprising the nucleic acid sequence SEQ ID NO. 62717, preferably the coding region thereof, conferred the production of or the increase in coenzyme Q9 compared with the wild type control.

The nucleic acid sequence of Ycl027w from Saccharomyces cerevisiae, e.g. as shown in the respective line in column 5 of Table I, application no. 16, has been published in A. And the activity of the gene product thereof is the activity of ycl027w-protein.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “ycl027w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said Ycl027w, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said Ycl027w, and preferably the activity is increased plastidic, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said Ycl027w, or a functional equivalent or a homolog thereof as depicted in column 8 of Table II, application no. 16, preferably a homolog or functional equivalent as depicted in column 8 of Table II B, application no. 16, and being depicted in the same respective line as said Ycl027w, and preferably the activity is increased plastidic, whereby the respective line disclose in column 7 the fine chemical coenzyme Q10.

Accordingly, in one embodiment, the molecule which activity is to be increased in the process of the invention is the gene product with an activity as a “ycl027w-protein”, preferably it is the molecule of section (a) or (b) of this paragraph.

In particular, it was observed that in plants, especially in Arabidopsis thaliana, increasing or generating the activity of a gene product plastidic with the activity of a “ycl027w-protein”, preferably being encoded by a gene comprising the nucleic acid sequence SEQ ID NO. 96980, preferably the coding region thereof, conferred the production of or the increase in coenzyme Q10 compared with the wild type control.

The nucleic acid sequence of Ydl159w from Saccharomyces cerevisiae, e.g. as shown in the respective line in column 5 of Table I, application no. 16, has been published in A. And the activity of the gene product thereof is the activity of protein kinase.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “protein kinase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said Ydl159w, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said Ydl159w, and preferably the activity is increased plastidic, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said Ydl159w, or a functional equivalent or a homolog thereof as depicted in column 8 of Table II, application no. 16, preferably a homolog or functional equivalent as depicted in column 8 of Table II B, application no. 16, and being depicted in the same respective line as said Ydl159w, and preferably the activity is increased plastidic, whereby the respective line disclose in column 7 the fine chemical coenzyme Q9.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product plastidic with the activity of a “protein kinase”, preferably being encoded by a gene comprising the nucleic acid sequence SEQ ID NO. 14275, preferably the coding region thereof, conferred the production of or the increase in coenzyme Q9 compared with the wild type control.

The nucleic acid sequence of Ykr072c from Saccharomyces cerevisiae, e.g. as shown in the respective line in column 5 of Table I, application no. 16, has been published in A. And the activity of the gene product thereof is the activity of halotolerance protein.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “halotolerance protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said Ykr072c, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said Ykr072c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said Ykr072c, or a functional equivalent or a homolog thereof as depicted in column 8 of Table II, application no. 16, preferably a homolog or functional equivalent as depicted in column 8 of Table II B, application no. 16, and being depicted in the same respective line as said Ykr072c, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “halotolerance protein”, preferably it is the molecule of section (a) or (b) of this paragraph.

In particular, it was observed that in plants, especially in Arabidopsis thaliana, increasing or generating the activity of a gene product non-targeted with the activity of a “halotolerance protein”, preferably being encoded by a gene comprising the nucleic acid sequence SEQ ID NO. 130903, preferably the coding region thereof, conferred the production of or the increase in coenzyme Q9 compared with the wild type control.

The nucleic acid sequence of Ykr089c from Saccharomyces cerevisiae, e.g. as shown in the respective line in column 5 of Table I, application no. 16, has been published in A. And the activity of the gene product thereof is the activity of lipase.

Accordingly, in one embodiment, the process of the present invention for producing nicotinic acid in a non-human organism, like a microorganism or a plant or a part thereof, comprises increasing or generating the activity of a gene product with the activity of a gene product conferring the activity of “lipase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical nicotinic acid), application no. 16, preferably the coding region thereof, or a homolog or a fragment thereof, and being depicted in the same respective line as said Ykr089c, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said Ykr089c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said Ykr089c, or a functional equivalent or a homolog thereof as depicted in column 8 of Table II, application no. 16, preferably a homolog or functional equivalent as depicted in column 8 of Table II B, application no. 16, and being depicted in the same respective line as said Ykr089c, and preferably the activity is increased non-targeted, whereby the respective line disclose in column 7 the fine chemical nicotinic acid.

Accordingly, in one embodiment, the molecule which activity is to be increased in the process of the invention is the gene product with an activity as a “lipase”, preferably it is the molecule of section (a) or (b) of this paragraph.

In particular, it was observed that in plants, especially in Arabidopsis thaliana, increasing or generating the activity of a gene product non-targeted with the activity of a “lipase”, preferably being encoded by a gene comprising the nucleic acid sequence SEQ ID NO. 102061, preferably the coding region thereof, conferred the production of or the increase in nicotinic acid compared with the wild type control.

The nucleic acid sequence of Ynl007c from Saccharomyces cerevisiae, e.g. as shown in the respective line in column 5 of Table I, application no. 16, has been published in A. And the activity of the gene product thereof is the activity of heat shock protein Hsp40.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “heat shock protein Hsp40”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said Ynl007c, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said Ynl007c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said Ynl007c, or a functional equivalent or a homolog thereof as depicted in column 8 of Table II, application no. 16, preferably a homolog or functional equivalent as depicted in column 8 of Table II B, application no. 16, and being depicted in the same respective line as said Ynl007c, and preferably the activity is increased non-targeted, whereby the respective line disclose in column 7 the fine chemical coenzyme Q10.

Accordingly, in one embodiment, the molecule which activity is to be increased in the process of the invention is the gene product with an activity as a “heat shock protein Hsp40”, preferably it is the molecule of section (a) or (b) of this paragraph.

In particular, it was observed that in plants, especially in Arabidopsis thaliana, increasing or generating the activity of a gene product non-targeted with the activity of a “heat shock protein Hsp40”, preferably being encoded by a gene comprising the nucleic acid sequence SEQ ID NO. 139173, preferably the coding region thereof, conferred the production of or the increase in coenzyme Q10 compared with the wild type control.

The nucleic acid sequence of Ynl007c from Saccharomyces cerevisiae, e.g. as shown in the respective line in column 5 of Table I, application no. 16, has been published in A. And the activity of the gene product thereof is the activity of heat shock protein Hsp40.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “heat shock protein Hsp40”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said Ynl007c, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said Ynl007c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said Ynl007c, or a functional equivalent or a homolog thereof as depicted in column 8 of Table II, application no. 16, preferably a homolog or functional equivalent as depicted in column 8 of Table II B, application no. 16, and being depicted in the same respective line as said Ynl007c, and preferably the activity is increased non-targeted, whereby the respective line disclose in column 7 the fine chemical coenzyme Q9.

Accordingly, in one embodiment, the molecule which activity is to be increased in the process of the invention is the gene product with an activity as a “heat shock protein Hsp40”, preferably it is the molecule of section (a) or (b) of this paragraph.

In particular, it was observed that in plants, especially in Arabidopsis thaliana, increasing or generating the activity of a gene product non-targeted with the activity of a “heat shock protein Hsp40”, preferably being encoded by a gene comprising the nucleic acid sequence SEQ ID NO. 139173, preferably the coding region thereof, conferred the production of or the increase in coenzyme Q9 compared with the wild type control.

The nucleic acid sequence of Yor244w from Saccharomyces cerevisiae, e.g. as shown in the respective line in column 5 of Table I, application no. 16, has been published in A. And the activity of the gene product thereof is the activity of histone acetyltransferase.

Accordingly, in one embodiment, the process of the present invention for producing coenzyme Q9 in a non-human organism, like a microorganism or a plant or a part thereof, comprises increasing or generating the activity of a gene product with the activity of a gene product conferring the activity of “histone acetyltransferase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q9), application no. 16, preferably the coding region thereof, or a homolog or a fragment thereof, and being depicted in the same respective line as said Yor244w, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said Yor244w, and preferably the activity is increased plastidic, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said Yor244w, or a functional equivalent or a homolog thereof as depicted in column 8 of Table II, application no. 16, preferably a homolog or functional equivalent as depicted in column 8 of Table II B, application no. 16, and being depicted in the same respective line as said Yor244w, and preferably the activity is increased plastidic, whereby the respective line disclose in column 7 the fine chemical coenzyme Q9.

Accordingly, in one embodiment, the molecule which activity is to be increased in the process of the invention is the gene product with an activity as a “histone acetyltransferase”, preferably it is the molecule of section (a) or (b) of this paragraph.

In particular, it was observed that in plants, especially in Arabidopsis thaliana, increasing or generating the activity of a gene product plastidic with the activity of a “histone acetyltransferase”, preferably being encoded by a gene comprising the nucleic acid sequence SEQ ID NO. 92540, preferably the coding region thereof, conferred the production of or the increase in coenzyme Q9 compared with the wild type control.

The nucleic acid sequence of Ypr054w from Saccharomyces cerevisiae, e.g. as shown in the respective line in column 5 of Table I, application no. 16, has been published in A. And the activity of the gene product thereof is the activity of mitogen-activated protein kinase.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “mitogen-activated protein kinase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said Ypr054w, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said Ypr054w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said Ypr054w, or a functional equivalent or a homolog thereof as depicted in column 8 of Table II, application no. 16, preferably a homolog or functional equivalent as depicted in column 8 of Table II B, application no. 16, and being depicted in the same respective line as said Ypr054w, and preferably the activity is increased non-targeted, whereby the respective line disclose in column 7 the fine chemical coenzyme Q10.

Accordingly, in one embodiment, the molecule which activity is to be increased in the process of the invention is the gene product with an activity as a “mitogen-activated protein kinase”, preferably it is the molecule of section (a) or (b) of this paragraph.

In particular, it was observed that in plants, especially in Arabidopsis thaliana, increasing or generating the activity of a gene product non-targeted with the activity of a “mitogen-activated protein kinase”, preferably being encoded by a gene comprising the nucleic acid sequence SEQ ID NO. 140110, preferably the coding region thereof, conferred the production of or the increase in coenzyme Q10 compared with the wild type control.

The nucleic acid sequence of ZM06LC1143 from Zea mays, e.g. as shown in the respective line in column 5 of Table I, application no. 16, is unpublished. And the activity of the gene product thereof is the activity of phosphopantothenoylcysteine decarboxylase.

Accordingly, in one embodiment, the process of the present invention for producing coenzyme Q10 in a non-human organism, like a microorganism or a plant or a part thereof, comprises increasing or generating the activity of a gene product with the activity of a gene product conferring the activity of “phosphopantothenoylcysteine decarboxylase”, especially from Zea mays or its functional equivalent or its homolog, e.g. the increase of
(a) a gene product of a gene comprising the nucleic acid molecule as shown in the respective line in column 5 of Table I (whereby the respective line disclose in column 7 the fine chemical coenzyme Q10), application no. 16, preferably the coding region thereof, or a homolog or a fragment thereof, and being depicted in the same respective line as said ZM06LC1143, or a functional equivalent or a homolog thereof as shown in column 8 of Table I, application no. 16, preferably the coding region thereof, particularly a homolog or functional equivalent as shown in column 8 of Table I B, application no. 16, and being depicted in the same respective line as said ZM06LC1143, and preferably the activity is increased non-targeted, or
(b) a polypeptide comprising a polypeptide, a consensus sequence or at least a polypeptide motif as shown in the respective line in column 5 of Table II or in column 8 of Table IV, application no. 16, respectively, and being depicted in the same respective line as said ZM06LC1143, or a functional equivalent or a homolog thereof as depicted in column 8 of Table II, application no. 16, preferably a homolog or functional equivalent as depicted in column 8 of Table II B, application no. 16, and being depicted in the same respective line as said ZM06LC1143, and preferably the activity is increased non-targeted, whereby the respective line disclose in column 7 the fine chemical coenzyme Q10.

Accordingly, in one embodiment, the molecule which activity is to be increased in the process of the invention is the gene product with an activity as a “phosphopantothenoylcysteine decarboxylase”, preferably it is the molecule of section (a) or (b) of this paragraph.

In particular, it was observed that in plants, especially in Arabidopsis thaliana, increasing or generating the activity of a gene product non-targeted with the activity of a “phosphopantothenoylcysteine decarboxylase”, preferably being encoded by a gene comprising the nucleic acid sequence SEQ ID NO. 68132, preferably the coding region thereof, conferred the production of or the increase in coenzyme Q10 compared with the wild type control.

[0105.1.7.16] to [0107.1.7.16] for the disclosure of these paragraphs see [0105.1.7.7] to [0107.1.7.7] above

A protein having an activity conferring an increase in the amount or level of the fine chemical coenzyme Q10, coenzyme Q9, lipoic acid, or nicotinic acid, upon targeting to the plastids or mitochondria or upon non-targeting, preferably has the structure of the respective polypeptide described herein, in particular of the polypeptides comprising the consensus sequence or at least one polypeptide motifs as shown in the respective line in Table IV, application no. 16, column 8 or of the polypeptide comprising an amino acid sequence as disclosed in the respective line in Table II, application no. 16, columns 5 or 8, or homologs or fragments thereof as described herein, or is encoded by the nucleic acid molecule characterized herein or the nucleic acid molecule according to the invention, for example by the nucleic acid molecule as shown in the respective line in Table I, application no. 16, columns 5 or 8, preferably the coding region thereof, or homologs or fragments thereof and has the herein mentioned activity.

for the disclosure of this paragraph see [0109.1.7.7] above

for the disclosure of this paragraph see [0110.1.7.7] above

In one embodiment, the process of the present invention comprises one or more of the following steps
(a) stabilizing a protein conferring the generation or increased expression of a protein encoded by the nucleic acid molecule of the invention or of the polypeptide of the invention, e.g. of a polypeptide having the activity selected from the group consisting of 2-C-methyl-D-erythritol 4-phosphate cytidylyltransferase, acetolactate synthase III large subunit, acetyltransferase, acid shock protein, amino acid ABC transporter permease protein, amino acid acetyltransferase, At1g47380-protein, ATP synthase subunit E, ATP-binding component of a transport system, auxin response factor, AX653549-protein, AY087308-protein, b1003-protein, b1097-protein, b1522-protein, b2032-protein, b2739-protein, b2849-protein, b3817-protein, b4029-protein, beta-galactosidase, calcium-dependent protein kinase, cation-transporting ATPase, CCAAT-binding transcription factor, cell division control protein, colanic acid biosynthesis protein, CTP synthase, cyclin D , disease resistance protein, enoyl-CoA hydratase, fatty acid desaturase, F-box protein, flavodoxin, gluconate transport system permease 3, glutamine amidotransferase, glutaredoxin, glycerol dehydrogenase, glycerol-3-phosphate dehydrogenase, glycosyl transferase, halotolerance protein, heat shock protein Hsp40, heat shock transcription factor, histone acetyltransferase, hydrolase, isopropylmalate isomerase large subunit, L-aspartate oxidase, lipase, MADS box protein transcription factor, malate dehydrogenase, methylglyoxal synthase, methyltransferase, mitogen-activated protein kinase, monothiol glutaredoxin, multiple antibiotic resistance protein, N-acetyl-gamma-glutamylphosphate reductase, NADH dehydrogenase I chain I, oxidoreductase, permease protein of ABC transporter, phenylalanine ammonia-lyase, phosphoadenosine phosphosulfate reductase, phosphopantothenoylcysteine decarboxylase, photosystem II protein, protein kinase, protein phosphatase, pyruvate kinase, recombinase A, Ribose import ATP-binding protein, Sec-independent protein translocase subunit, serine protease, serine/threonine-protein phosphatase, sll1761-protein, slr1107-protein, sterol desaturase, threonine synthase, transcription factor, transcriptional regulator, TTC1386-protein, uroporphyrin-III C-methyltransferase, ycl027w-protein, zinc finger protein, and zinc transporter, or of a polypeptide as indicated in the respective line in Table II, application no. 16, columns 5 or 8, or its homologs or fragments, and conferring the production of or an increase in coenzyme Q10, coenzyme Q9, lipoic acid, or nicotinic acid, respectively; and/or
(b) stabilizing a mRNA conferring the generation or increased expression of a FCRP, e.g. protein encoded by the nucleic acid molecule of the invention or its homologs or fragments, or of a mRNA encoding the polypeptide of the present invention having the hereinmentioned activity selected from the group consisting of said activities mentioned in (a) and conferring the production of or an increase in coenzyme Q10, coenzyme Q9, lipoic acid, or nicotinic acid, respectively; and/or
(c) increasing the specific activity of a protein conferring the increased expression of a FCRP, e.g. a protein encoded by the nucleic acid molecule of the invention or of the polypeptide of the present invention having herein-mentioned coenzyme Q10, coenzyme Q9, lipoic acid, or nicotinic acid generating or increasing activity, respectively, e.g. of a polypeptide having the activity of a protein as indicated in the respective line in Table II, application no. 16, columns 5 or 8, or its homologs or fragments, or decreasing the inhibitory regulation of the polypeptide of the invention; and/or
(d) generating or increasing the expression of an endogenous or artificial transcription factor mediating the expression of a protein conferring the generation or increased expression of a FCRP, e.g. a protein encoded by the nucleic acid molecule of the invention or of the polypeptide of the invention having herein-mentioned activity selected from the group consisting of said activities mentioned in (a) and conferring a coenzyme Q10, coenzyme Q9, lipoic acid, or nicotinic acid increasing activity, respectively, e.g. of a polypeptide having the activity of a protein as indicated in the respective line in Table II, application no. 16, columns 5 or 8 or its homologs; and/or
(e) stimulating activity of a protein conferring the increased expression of a FCRP, e.g. a protein encoded by the nucleic acid molecule of the present invention or a polypeptide of the present invention having herein-mentioned activity selected from the group consisting of said activities mentioned in (a) and conferring a coenzyme Q10, coenzyme Q9, lipoic acid, or nicotinic acid increasing activity, respectively, e.g. of a polypeptide having the activity of a protein as indicated in the respective line in Table II, application no. 16, columns 5 or 8, or its homologs or fragments, by adding one or more exogenous inducing factors to the non-human organism or parts thereof; and/or
(f) expressing a transgenic gene encoding a protein conferring the increased expression of a FCRP, e.g. a polypeptide encoded by the nucleic acid molecule of the present invention or a polypeptide of the present invention, having herein-mentioned activity selected from the group consisting of said activities mentioned in (a) and conferring a coenzyme Q10, coenzyme Q9, lipoic acid, or nicotinic acid increasing activity, respectively, e.g. of a polypeptide having the activity of a protein as indicated in the respective line in Table II, application no. 16, columns 5 or 8, or its homologs or fragments, and/or
(g) increasing the copy number of a gene conferring the increased expression of a nucleic acid molecule encoding a FCRP, e.g. a polypeptide encoded by the nucleic acid molecule of the invention or the polypeptide of the invention having herein-mentioned activity selected from the group consisting of said activities mentioned in (a) and conferring a coenzyme Q10, coenzyme Q9, lipoic acid, or nicotinic acid increasing activity, respectively, e.g. of a polypeptide having the activity of a protein as indicated in the respective line in Table II, application no. 16, columns 5 or 8, or its homologs or fragments; and/or
(h) increasing the expression of the endogenous gene encoding the FCRP, polypeptide of the invention, e.g. a polypeptide encoded by the nucleic acid molecule of the invention or the polypeptide of the invention having herein-mentioned activity selected from the group consisting of said activities mentioned in (a) and conferring a coenzyme Q10, coenzyme Q9, lipoic acid, or nicotinic acid; increasing activity, respectively, e.g. of a polypeptide having the activity of a protein as indicated in the respective line in Table II, application no. 16, columns 5 or 8, or its homologs or fragments, by adding positive expression or removing negative expression elements, e.g. homologous recombination can be used to either introduce positive regulatory elements like for plants the 35S enhancer into the promoter or to remove repressor elements form regulatory regions. Further gene conversion methods can be used to disrupt repressor elements or to enhance to activity of positive elements. Positive elements can be randomly introduced in plants by T-DNA or transposon mutagenesis and lines can be identified in which the positive elements have be integrated near to a gene of the invention, the expression of which is thereby enhanced; and/or
(i) modulating growth conditions of the non-human organism in such a manner, that the expression or activity of the gene encoding the FCRP, e.g. a protein of the invention or the protein itself is enhanced, for example microorganisms or plants can be grown for example under a higher temperature regime leading to an enhanced expression of heat shock proteins, which can lead to an enhanced coenzyme Q10, coenzyme Q9, lipoic acid, or nicotinic acid production, respectively; and/or
(j) selecting of non-human organisms with especially high activity of the proteins of the invention from natural or from mutagenized resources and breeding them into the target organisms, e.g. the elite crops; and/or
(k) directing a protein encoded by the nucleic acid molecule of the invention or of the polypeptide of the present invention having herein-mentioned activity selected from the group consisting of said activities mentioned in (a) and conferring a coenzyme Q10, coenzyme Q9, lipoic acid, or nicotinic acid increasing activity, respectively, e.g. of a polypeptide having the activity of a protein as indicated in the respective line in Table II, application no. 16, columns 5 or 8, or its homologs or fragments, if for the corresponding nucleic acid molecule in the respective line in column 6 of Table I the term “plastidic” is indicated, to the plastids by the addition of a plastidial targeting sequence or if for the corresponding nucleic acid molecule in the respective line in column 6 of Table I the term “mitochondric” is indicated, to the mitochondria by the addition of a mitochondrial targeting sequence; and/or
(l) generating the expression of a protein encoded by the nucleic acid molecule of the invention or of the polypeptide of the present invention having herein-mentioned activity selected from the group consisting of said activities mentioned in (a) and conferring a coenzyme Q10, coenzyme Q9, lipoic acid, or nicotinic acid increasing activity, respectively, e.g. of a polypeptide having the activity of a protein as indicated in the respective line in Table II, application no. 16, columns 5 or 8, or its homologs or fragments, if for the corresponding nucleic acid molecule in the respective line in column 6 of Table I the term “plastidic” or “mitochondric” is indicated, in these organelles by the stable or transient transformation, advantageously stable transformation, of organelles, preferably plastids or mitochondria, with an inventive nucleic acid sequence preferably in form of an expression cassette containing said sequence leading to the expression of the nucleic acids or polypeptides of the invention in the respective organelle; and/or
(m) generating the expression of a protein encoded by the nucleic acid molecule of the invention or of the polypeptide of the present invention having herein-mentioned activity selected from the group consisting of said activities mentioned in (a) and conferring a coenzyme Q10, coenzyme Q9, lipoic acid, and/or nicotinic acid increasing activity, respectively, e.g. of a polypeptide having the activity of a protein as indicated in the respective line in Table II, application no. 16, columns 5 or 8, or its homologs or fragments, if for the corresponding nucleic acid molecule in the respective line in column 6 of Table I the term “plastidic” or “mitochondric” is indicated, in these organelles by integration of a nucleic acid of the invention into the genome of the respective organelle under control of preferable a promoter selective for the respective organelle.

Preferably, said mRNA is the coding region of the nucleic acid molecule of the present invention and/or the protein conferring the increased expression of a protein encoded by the coding region of the nucleic acid molecule of the present invention alone or linked to a transit nucleic acid sequence or transit peptide encoding nucleic acid sequence or the polypeptide having the herein mentioned activity, e.g. conferring the generation of or increase of coenzyme Q10, coenzyme Q9, lipoic acid, or nicotinic acid, respectively, after increasing the expression or activity of the encoded polypeptide, non-targeted or in organelles such as plastids and/or mitochondria, preferably plastids, or having the activity of a polypeptide having an activity as the protein as shown in the respective line in Table II, application no. 16, column 3, or its homologs. Preferably the increase of coenzyme 010, coenzyme Q9, lipoic acid, or nicotinic acid, respectively, takes place non-targeted or in plastids and/or mitochondria, preferably non-targeted or in plastids.

[0113.1.7.16] to [0122.1.7.16] for the disclosure of these paragraphs see [0113.1.7.7] to [0122.1.7.7] above

The activation of an endogenous polypeptide having above-mentioned activity, e.g. having the activity of a protein as shown in the respective line in Table II, application no. 16, column 3 or of the polypeptide of the invention, e.g. conferring the increase of the fine chemical coenzyme Q10, coenzyme Q9, lipoic acid, or nicotinic acid, respectively, by increase of expression or activity in the cytoplasm, and/or in the cytosol, and/or in an organelle, such as plastids or mitochondria, can also be increased by introducing a synthetic transcription factor, which binds close to the coding region of the gene encoding the protein as shown in the respective line in Table II, application no. 16, column 5 or 8, or homologs or fragments thereof, and activates its transcription. A chimeric zinc finger protein can be constructed, which comprises a specific DNA-binding domain and an activation domain as e.g. the VP16 domain of Herpes Simplex virus. The specific binding domain can bind to the regulatory region of the gene encoding the protein as shown in the respective line in Table II, application no. 16, column 5 or 8, or homologs or fragments thereof. The expression of the chimeric transcription factor in a non-human organism, in particular in a plant, leads to a specific expression of the protein as shown in the respective line in Table II, application no. 16, column 5 or 8, or homologs or fragments thereof, see e.g. in WO01/52620, Oriz, Proc. Natl. Acad. Sci. USA 99, 13290 (2002), or Guan, Proc. Natl. Acad. Sci. USA 99, 13296 (2002).

[0124.1.7.16] to [0127.1.7.16] for the disclosure of these paragraphs see [0124.1.7.7] to [0127.1.7.7] above

Owing to the introduction of a gene or a plurality of genes conferring the expression of the nucleic acid molecule of the invention or the polypeptide of the invention, for example the nucleic acid construct mentioned below, or encoding the protein as shown in the respective line in Table II, application no. 16, column 5 or 8, or homologs or fragments thereof, into a non-human organism alone or in combination with other genes, it is possible not only to increase the biosynthetic flux towards the end product, but also to increase, modify or create de novo an advantageous, preferably novel metabolites composition in the non-human organism, e.g. an advantageous composition comprising a higher content of (from a viewpoint of nutritional physiology limited) coenzyme Q10, coenzyme Q9, lipoic acid, or nicotinic acid and if desired other cofactors, and/or other metabolites, in free or bound form.

for the disclosure of this paragraph see [0129.1.7.7] above

Accordingly, in one embodiment, the process according to the invention relates to a process, which comprises:
(a) providing a non-human organism, preferably a microorganism, a plant cell, a plant tissue, a plant or a part thereof, more preferably a microorganism, a plant tissue, a plant or a part thereof;
(b) increasing the activity of a protein as shown in the respective line in Table II, application no. 16, column 3 or of a polypeptide being encoded by the respective nucleic acid molecule of the present invention and described below, e.g. conferring an increase of the fine chemical coenzyme Q10, coenzyme Q9, lipoic acid, or nicotinic acid, respectively, in the non-human organism, preferably in the microorganism, the plant cell, the plant tissue, the plant or a part thereof, more preferably a microorganism, a plant tissue, a plant or a part thereof, especially cytoplasmic or in an organelle, like plastids or mitochondria,
(c) growing the non-human organism, preferably the microorganism, the plant cell, the plant tissue, the plant or a part thereof, under conditions which permit the production of said chemical in the non-human organism, preferably the microorganism, the plant cell, the plant tissue, the plant or a part thereof; and
(d) if desired, recovering, optionally isolating, said fine chemical, in free and/or bound form, and, optionally further free and/or bound cofactors, and/or other metabolites synthesized by the non-human organism, the microorganism, the plant cell, the plant tissue, the plant or a part thereof.

[0131.1.7.16] to [0133.1.7.16] for the disclosure of these paragraphs see [0131.1.7.7] to [0133.1.7.7] above

The non-human organism, in particular the microorganism, plant or part thereof, is advantageously grown in such a way that it is not only possible to recover, if desired to isolate the free or bound fine chemical or the free and bound fine chemical but as option it is also possible to produce, recover and, if desired to isolate, other free or/and bound vitamins and/or other metabolites.

The organisms are harvested in a manner known per se and appropriate for the particular organism. Microorganisms such as bacteria, mosses, yeasts and fungi or plant cells which are cultured in liquid media by fermentation may be removed, for example, by centrifugation, decanting or filtration. Plants are grown on solid media in a manner known per se and harvested accordingly.

The organisms, its cells or a part thereof or the recovered, and if desired isolated, cofactors, in particular coenzyme Q10, coenzyme Q9, lipoic acid, and/or nicotinic acid, and/or protein(s) such as enzyme(s), containing coenzyme 010, coenzyme Q9, lipoic acid, and/or nicotinic acid can then be processed further directly into foodstuffs or animal feeds or for other applications, for example according to the disclosures made in WO/2005/083093 or WO2005/012316, which are expressly incorporated herein by reference.

The essentially chemically pure cofactors comprising compositions, in particular the essentially pure coenzyme Q10, coenzyme Q9, lipoic acid, and/or nicotinic acid and/or protein(s) such as enzyme(s) containing coenzyme Q10, coenzyme Q9, lipoic acid, and/or nicotinic acid and/or coenzyme Q10, coenzyme Q9, lipoic acid, and/or nicotinic acid comprising compositions, are advantageous for applications in the field of the food industry, the cosmetics industry or the pharmaceutical industry. Said compositions can be combined with other fine chemicals, e.g. nutrients or food or feed additives, such as fatty acids, vitamins, provitamins, antioxidants, carotenoids, amino acids, trace elements etc.

The organism, in particular the microorganism, non-human animal, the plant or animal cell, the plant or animal tissue or the plant is advantageously grown in such a way that it is not only possible to recover, if desired isolate the free or bound the fine chemical or the free and bound the fine chemical but as option it is also possible to produce, recover and, if desired isolate, other free or/and bound cofactors such as Coenzyme Q0 to Q8, and/or trigonelleine resp. or mixtures thereof.

The organism such as microorganisms or plants or the recovered, and if desired isolated, fine chemical can then be processed further directly into foodstuffs or animal feeds or for other applications, for example according to the disclosures made in the following US patent publications:

U.S. Pat. No. 6,232,346: Composition for improvement of cellular nutrition and mitochondrial energetics, U.S. Pat. No. 6,228,347: Antioxidant gel for gingival conditions, U.S. Pat. No. 6,203,818: Nutritional supplement for cardiovascular health, U.S. Pat. No. 6,200,550: Oral care compositions comprising coenzyme Q10, US 6,191,172: Water-soluble compositions of bioactive lipophilic compounds , U.S. Pat. No. 6,184,255: Pharmaceutical composition comprising coenzyme Q10, U.S. Pat. No. 6,080,788: Composition for improvement of cellular nutrition and mitochondrial energetics, U.S. Pat. No. 6,069,167: Use of antioxidant agents to treat cholestatic liver disease, U.S. Pat. No. 6,063,820: Medical food for diabetics, U.S. Pat. No. 6,054,261: Coenzyme Q.sub.10 compositions for organ protection during perfusion, U.S. Pat. No. 6,051,250: Process for the stabilization of vesicles of amphiphilic lipid(s) and composition for topical application containing the said stabilized vesicles.

[0135.1.7.16] to [0139.1.7.16] for the disclosure of these paragraphs see [0135.1.7.7] to [0139.1.7.7] above

In another embodiment, the invention relates to a (isolated) nucleic acid molecule, which comprises a nucleic acid molecule selected from the group consisting of:
(a) a nucleic acid molecule encoding a polypeptide shown in Table II, application no. 16, column 5 or 8, preferably shown in Table II B, application no. 16, column 8, or a homolog or a fragment thereof;
(b) a nucleic acid molecule shown in Table I, application no. 16, column 5 or 8, preferably shown in Table I B, application no. 16, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 16, preferably in column 8 of Table II B, application no. 16;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 16, preferably shown in column 8 of Table I B, application no. 16, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 16;
(i) a nucleic acid molecule encoding a polypeptide having the activity represented by a respective protein as depicted in column 5 of Table II, application no. 16;
(j) a nucleic acid molecule comprising a polynucleotide which is obtained by amplifying a cDNA library or a genomic library using the primers in Table III, application no. 16, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 16.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II application no. 16 and confers a generation or an increase of the respective fine chemical in a non-human organism or a part thereof as compared to a corresponding wild-type non-human organism or part thereof.

In another embodiment, the invention relates to a (isolated) nucleic acid molecule, which comprises a nucleic acid molecule selected from the group consisting of:
(a) a nucleic acid molecule encoding a polypeptide shown in Table II, column 5 or 8, application no. 16, preferably shown in Table II A, application no. 16, in column 5 or in Table II A, application no. 16, column 8 or in Table II B, application no. 16, column 8, or a homolog or a fragment thereof;
(b) a nucleic acid molecule shown in Table I, application no. 16, column 5 or 8, preferably shown in Table I A, application no. 16, in column 5 or in Table I A, application no. 16, column 8 or in Table I B, application no. 16, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 16, preferably shown in Table II A, application no. 16, in column 5 or in Table II A, application no. 16, column 8 or in Table II B, application no. 16, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 16, preferably shown in Table I A, application no. 16, in column 5 or in Table I A, application no. 16, column 8 or in Table I B column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 16;
(i) a nucleic acid molecule encoding a polypeptide having the activity represented by a respective protein as depicted in column 5 of Table II, application no. 16;
(j) a nucleic acid molecule comprising a polynucleotide which is obtained by amplifying a cDNA library or a genomic library using the primers in Table III, application no. 16, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt,500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto;
   whereby, preferably, the nucleic acid molecule according to (a), (b), (c), (d), (e), (f), (g), (h), (i), (j) or (k) distinguishes over the sequence depicted in Table I A and/or I B, application no. 16, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 16, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 16, column 5 or 8, or the coding region thereof, respectively. In a further embodiment the nucleic acid molecule does not encode the polypeptide sequence shown in Table II A and/or II B, application no. 16, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 16, column 5 or 8 and does not encode a protein of the sequence shown in Table II A and/or II B, application no. 16, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 16, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 16, column 5 or 8 but less than 100%, preferably less than 99.999%, 99.99%, 99.9%, 99%, 98%, 97%, 96% or 95% identical to the sequence shown in Table II A and/or II B, application no. 16, column 5 or 8.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 16.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II, application no. 16, and confers a generation or an increase of the respective fine chemical in a non-human organism or a part thereof as compared to a corresponding wild-type non-human organism or part thereof.

[0144.1.7.16] to [0155.1.7.16] for the disclosure of these paragraphs see [0144.1.7.7] to [0155.1.7.7] above

In a preferred embodiment a nucleic acid construct, for example an expression cassette, comprises upstream, i.e. at the 5′ end of the encoding sequence, a promoter and downstream, i.e. at the 3′ end, a polyadenylation signal and optionally other regulatory elements which are operably linked to the intervening encoding sequence with one of the nucleic acids as depicted in the respective line in Table I, application no. 16, column 5 or 8, preferably the coding region thereof, or a homolog or a fragment thereof. By an operable linkage is meant the sequential arrangement of promoter, encoding sequence, terminator, optionally other regulatory elements and optionally targeting sequences in such a way that each of the regulatory elements/targeting sequence can fulfill its function in the expression of the encoding sequence in due manner. In one embodiment the sequences preferred for operable linkage are targeting sequences for ensuring subcellular localization in organelles, like plastids or mitochondria.

However, targeting sequences for ensuring subcellular localization in the endoplasmic reticulum (=ER), in the nucleus, in oil corpuscles or other compartments may also be employed as well as translation promoters such as the 5′ lead sequence in tobacco mosaic virus (Gallie et al., Nucl. Acids Res. 15, 8693 (1987)).

for the disclosure of this paragraph see [0157.1.7.7] above

In an embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 16, column 5 or 8, preferably shown in Table II A, application no. 16, column 5, or in Table II A, application no. 16, column 8, or in Table II B, application no. 16, column 8, or a homolog or a fragment thereof;
   (b) a nucleic acid molecule shown in Table I, application no. 16, column 5 or 8, preferably shown in Table I A, application no. 16, column 5, or in Table I A, application no. 16, column 8, or in Table I B, application no. 16, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 16, preferably shown in Table II A, application no. 16, column 5, or in Table II A, application no. 16, column 8, or in Table II B, application no. 16, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 16, preferably shown in Table I A, application no. 16, column 5, or in Table I A, application no. 16, column 8, or in Table I B, application no. 16, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 16;
   (i) a nucleic acid molecule encoding a polypeptide having the activity represented by a respective protein as depicted in column 5 of Table II, application no. 16;
   (j) a nucleic acid molecule comprising a polynucleotide which is obtained by amplifying a cDNA library or a genomic library using the primers in Table III, application no. 16, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto. and, optionally
3) nucleic acid molecule encoding a transit peptide, preferably encoding a plastidal transit peptide or a mitochondrial transit peptide; which are operable linked.
   (All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 16.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 16 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 16, in the same line as of the respective nucleic acid molecule according to 2) “plastidic” is depicted, or encoding a mitochondrial transit peptide, especially in case in column 6 of Table I, application no. 16, in the same line as of the respective nucleic acid molecule according to 2) “mitochondric” is depicted.

In another embodiment thereof the expression cassette does not comprise 3) a nucleic acid molecule encoding a transit peptide, especially in case in column 6 of Table I, application no. 16, in the same line as of the respective nucleic acid molecule according to 2) “non-targeted” is depicted.

In a further embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 16, column 5 or 8, preferably shown in Table II A, application no. 16, column 5, or in Table II A, application no. 16, column 8, or in Table II B, application no. 16, column 8, or a homolog or a fragment thereof;
   (b) a nucleic acid molecule shown in Table I, application no. 16, column 5 or 8, preferably shown in Table I A, application no. 16, column 5, or in Table I A, application no. 16, column 8, or in Table I B, application no. 16, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 16, preferably shown in Table II A, application no. 16, column 5, or in Table II A, application no. 16, column 8, or in Table II B, application no. 16, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 16, preferably shown in Table I A, application no. 16, column 5, or in Table I A, application no. 16, column 8, or in Table I B, application no. 16, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 16;
   (i) a nucleic acid molecule encoding a polypeptide having the activity represented by a respective protein as depicted in column 5 of Table II, application no. 16;
   (j) a nucleic acid molecule comprising a polynucleotide which is obtained by amplifying a cDNA library or a genomic library using the primers in Table III, application no. 16, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto; and, optionally
3) nucleic acid molecule encoding a transit peptide, preferably encoding a plastidal transit peptide or a mitochondrial transit peptide;
   which are operable linked and
   whereby, preferably, the nucleic acid molecule according to 2 (a), (b), (c), (d), (e), (f), (g), (h), (i), (j) or (k) distinguishes over the sequence depicted in Table I A and/or I B, application no. 16, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention according to 2) does not consist of the sequence shown in Table I A and/or I B, application no. 16, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 16, column 5 or 8, or the coding region thereof, respectively. In a further embodiment the nucleic acid molecule does not encode the polypeptide sequence shown in Table II A and/or II B, application no. 16, column 5 or 8 but homologs thereof.

Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 16, column 5 or 8 and does not encode a protein of the sequence shown in Table II A and/or II B, application no. 16, column 5 or 8. Accordingly, in one embodiment, the protein encoded by a sequence of a nucleic acid according to 2(a), (b), (c), (d), (e), (f), (g), (h), (i), (j) or (k) does not consist of the sequence shown in Table I A and/or I B, application no. 16, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 16, column 5 or 8 but less than 100%, 99.999%, 99.99%, 99.9%, 99%, 98%, 97%, 96% or 95% identical to the sequence shown in Table II A and/or II B, application no. 16, column 5 or 8.

(All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 16.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 16 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 16, in the same line as of the respective nucleic acid molecule according to 2) “plastidic” is depicted, or encoding a mitochondrial transit peptide, especially in case in column 6 of Table I, application no. 16, in the same line as of the respective nucleic acid molecule according to 2) “mitochondric” is depicted.

In another embodiment thereof the expression cassette does not comprise 3) a nucleic acid molecule encoding a transit peptide, especially in case in column 6 of Table I, application no. 16, in the same line as of the respective nucleic acid molecule according to 2) “non-targeted” is depicted.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17356, or a homolog or fragment thereof, or a homolog thereof being depicted in Table I, application no. 16, column 8, in the same line as SEQ ID NO. 17356, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 17356 is depicted, plastidic is mentioned, or a mitochondrial transit peptide in case in Table I, application no. 16, in column 6 in the same line as SEQ ID NO. 17356 is depicted, mitochondric is mentioned; which is operable linked, too.

In another embodiment thereof the expression cassette does not comprise an additional transit peptide in case in Table I, application no. 16, in column 6 in the same line as SEQ ID NO. 17356 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 18070, or a homolog or fragment thereof, or a homolog thereof being depicted in Table I, application no. 16, column 8, in the same line as SEQ ID NO. 18070, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 18070 is depicted, plastidic is mentioned, or a mitochondrial transit peptide in case in Table I, application no. 16, in column 6 in the same line as SEQ ID NO. 18070 is depicted, mitochondric is mentioned; which is operable linked, too.

In another embodiment thereof the expression cassette does not comprise an additional transit peptide in case in Table I, application no. 16, in column 6 in the same line as SEQ ID NO. 18070 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 18122, or a homolog or fragment thereof, or a homolog thereof being depicted in Table I, application no. 16, column 8, in the same line as SEQ ID NO. 18122, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 18122 is depicted, plastidic is mentioned, or a mitochondrial transit peptide in case in Table I, application no. 16, in column 6 in the same line as SEQ ID NO. 18122 is depicted, mitochondric is mentioned; which is operable linked, too.

In another embodiment thereof the expression cassette does not comprise an additional transit peptide in case in Table I, application no. 16, in column 6 in the same line as SEQ ID NO. 18122 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 19419, or a homolog or fragment thereof, or a homolog thereof being depicted in Table I, application no. 16, column 8, in the same line as SEQ ID NO. 19419, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 19419 is depicted, plastidic is mentioned, or a mitochondrial transit peptide in case in Table I, application no. 16, in column 6 in the same line as SEQ ID NO. 19419 is depicted, mitochondric is mentioned; which is operable linked, too.

In another embodiment thereof the expression cassette does not comprise an additional transit peptide in case in Table I, application no. 16, in column 6 in the same line as SEQ ID NO. 19419 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 133042, or a homolog or fragment thereof, or a homolog thereof being depicted in Table I, application no. 16, column 8, in the same line as SEQ ID NO. 133042, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 133042 is depicted, plastidic is mentioned, or a mitochondrial transit peptide in case in Table I, application no. 16, in column 6 in the same line as SEQ ID NO. 133042 is depicted, mitochondric is mentioned; which is operable linked, too.

In another embodiment thereof the expression cassette does not comprise an additional transit peptide in case in Table I, application no. 16, in column 6 in the same line as SEQ ID NO. 133042 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 108727, or a homolog or fragment thereof, or a homolog thereof being depicted in Table I, application no. 16, column 8, in the same line as SEQ ID NO. 108727, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 108727 is depicted, plastidic is mentioned, or a mitochondrial transit peptide in case in Table I, application no. 16, in column 6 in the same line as SEQ ID NO. 108727 is depicted, mitochondric is mentioned; which is operable linked, too.

In another embodiment thereof the expression cassette does not comprise an additional transit peptide in case in Table I, application no. 16, in column 6 in the same line as SEQ ID NO. 108727 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 21159, or a homolog or fragment thereof, or a homolog thereof being depicted in Table I, application no. 16, column 8, in the same line as SEQ ID NO. 21159, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 21159 is depicted, plastidic is mentioned, or a mitochondrial transit peptide in case in Table I, application no. 16, in column 6 in the same line as SEQ ID NO. 21159 is depicted, mitochondric is mentioned; which is operable linked, too.

In another embodiment thereof the expression cassette does not comprise an additional transit peptide in case in Table I, application no. 16, in column 6 in the same line as SEQ ID NO. 21159 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68657, or a homolog or fragment thereof, or a homolog thereof being depicted in Table I, application no. 16, column 8, in the same line as SEQ ID NO. 68657, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 68657 is depicted, plastidic is mentioned, or a mitochondrial transit peptide in case in Table I, application no. 16, in column 6 in the same line as SEQ ID NO. 68657 is depicted, mitochondric is mentioned; which is operable linked, too.

In another embodiment thereof the expression cassette does not comprise an additional transit peptide in case in Table I, application no. 16, in column 6 in the same line as SEQ ID NO. 68657 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 21497, or a homolog or fragment thereof, or a homolog thereof being depicted in Table I, application no. 16, column 8, in the same line as SEQ ID NO. 21497, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 21497 is depicted, plastidic is mentioned, or a mitochondrial transit peptide in case in Table I, application no. 16, in column 6 in the same line as SEQ ID NO. 21497 is depicted, mitochondric is mentioned; which is operable linked, too.

In another embodiment thereof the expression cassette does not comprise an additional transit peptide in case in Table I, application no. 16, in column 6 in the same line as SEQ ID NO. 21497 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 120952, or a homolog or fragment thereof, or a homolog thereof being depicted in Table I, application no. 16, column 8, in the same line as SEQ ID NO. 120952, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 120952 is depicted, plastidic is mentioned, or a mitochondrial transit peptide in case in Table I, application no. 16, in column 6 in the same line as SEQ ID NO. 120952 is depicted, mitochondric is mentioned; which is operable linked, too.

In another embodiment thereof the expression cassette does not comprise an additional transit peptide in case in Table I, application no. 16, in column 6 in the same line as SEQ ID NO. 120952 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 22921, or a homolog or fragment thereof, or a homolog thereof being depicted in Table I, application no. 16, column 8, in the same line as SEQ ID NO. 22921, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 22921 is depicted, plastidic is mentioned, or a mitochondrial transit peptide in case in Table I, application no. 16, in column 6 in the same line as SEQ ID NO. 22921 is depicted, mitochondric is mentioned; which is operable linked, too.

In another embodiment thereof the expression cassette does not comprise an additional transit peptide in case in Table I, application no. 16, in column 6 in the same line as SEQ ID NO. 22921 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68777, or a homolog or fragment thereof, or a homolog thereof being depicted in Table I, application no. 16, column 8, in the same line as SEQ ID NO. 68777, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 68777 is depicted, plastidic is mentioned, or a mitochondrial transit peptide in case in Table I, application no. 16, in column 6 in the same line as SEQ ID NO. 68777 is depicted, mitochondric is mentioned; which is operable linked, too.

In another embodiment thereof the expression cassette does not comprise an additional transit peptide in case in Table I, application no. 16, in column 6 in the same line as SEQ ID NO. 68777 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 2367, or a homolog or fragment thereof, or a homolog thereof being depicted in Table I, application no. 16, column 8, in the same line as SEQ ID NO. 2367, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 2367 is depicted, plastidic is mentioned, or a mitochondrial transit peptide in case in Table I, application no. 16, in column 6 in the same line as SEQ ID NO. 2367 is depicted, mitochondric is mentioned; which is operable linked, too.

In another embodiment thereof the expression cassette does not comprise an additional transit peptide in case in Table I, application no. 16, in column 6 in the same line as SEQ ID NO. 2367 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 2935, or a homolog or fragment thereof, or a homolog thereof being depicted in Table I, application no. 16, column 8, in the same line as SEQ ID NO. 2935, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 2935 is depicted, plastidic is mentioned, or a mitochondrial transit peptide in case in Table I, application no. 16, in column 6 in the same line as SEQ ID NO. 2935 is depicted, mitochondric is mentioned; which is operable linked, too.

In another embodiment thereof the expression cassette does not comprise an additional transit peptide in case in Table I, application no. 16, in column 6 in the same line as SEQ ID NO. 2935 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 131208, or a homolog or fragment thereof, or a homolog thereof being depicted in Table I, application no. 16, column 8, in the same line as SEQ ID NO. 131208, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 131208 is depicted, plastidic is mentioned, or a mitochondrial transit peptide in case in Table I, application no. 16, in column 6 in the same line as SEQ ID NO. 131208 is depicted, mitochondric is mentioned; which is operable linked, too.

In another embodiment thereof the expression cassette does not comprise an additional transit peptide in case in Table I, application no. 16, in column 6 in the same line as SEQ ID NO. 131208 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 23482, or a homolog or fragment thereof, or a homolog thereof being depicted in Table I, application no. 16, column 8, in the same line as SEQ ID NO. 23482, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 23482 is depicted, plastidic is mentioned, or a mitochondrial transit peptide in case in Table I, application no. 16, in column 6 in the same line as SEQ ID NO. 23482 is depicted, mitochondric is mentioned; which is operable linked, too.

In another embodiment thereof the expression cassette does not comprise an additional transit peptide in case in Table I, application no. 16, in column 6 in the same line as SEQ ID NO. 23482 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 24311, or a homolog or fragment thereof, or a homolog thereof being depicted in Table I, application no. 16, column 8, in the same line as SEQ ID NO. 24311, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 24311 is depicted, plastidic is mentioned, or a mitochondrial transit peptide in case in Table I, application no. 16, in column 6 in the same line as SEQ ID NO. 24311 is depicted, mitochondric is mentioned; which is operable linked, too.

In another embodiment thereof the expression cassette does not comprise an additional transit peptide in case in Table I, application no. 16, in column 6 in the same line as SEQ ID NO. 24311 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4348, or a homolog or fragment thereof, or a homolog thereof being depicted in Table I, application no. 16, column 8, in the same line as SEQ ID NO. 4348, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 4348 is depicted, plastidic is mentioned, or a mitochondrial transit peptide in case in Table I, application no. 16, in column 6 in the same line as SEQ ID NO. 4348 is depicted, mitochondric is mentioned; which is operable linked, too.

In another embodiment thereof the expression cassette does not comprise an additional transit peptide in case in Table I, application no. 16, in column 6 in the same line as SEQ ID NO. 4348 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 24438, or a homolog or fragment thereof, or a homolog thereof being depicted in Table I, application no. 16, column 8, in the same line as SEQ ID NO. 24438, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 24438 is depicted, plastidic is mentioned, or a mitochondrial transit peptide in case in Table I, application no. 16, in column 6 in the same line as SEQ ID NO. 24438 is depicted, mitochondric is mentioned; which is operable linked, too.

In another embodiment thereof the expression cassette does not comprise an additional transit peptide in case in Table I, application no. 16, in column 6 in the same line as SEQ ID NO. 24438 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4904, or a homolog or fragment thereof, or a homolog thereof being depicted in Table I, application no. 16, column 8, in the same line as SEQ ID NO. 4904, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 4904 is depicted, plastidic is mentioned, or a mitochondrial transit peptide in case in Table I, application no. 16, in column 6 in the same line as SEQ ID NO. 4904 is depicted, mitochondric is mentioned; which is operable linked, too.

In another embodiment thereof the expression cassette does not comprise an additional transit peptide in case in Table I, application no. 16, in column 6 in the same line as SEQ ID NO. 4904 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 102223, or a homolog or fragment thereof, or a homolog thereof being depicted in Table I, application no. 16, column 8, in the same line as SEQ ID NO. 102223, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 102223 is depicted, plastidic is mentioned, or a mitochondrial transit peptide in case in Table I, application no. 16, in column 6 in the same line as SEQ ID NO. 102223 is depicted, mitochondric is mentioned; which is operable linked, too.

In another embodiment thereof the expression cassette does not comprise an additional transit peptide in case in Table I, application no. 16, in column 6 in the same line as SEQ ID NO. 102223 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 25498, or a homolog or fragment thereof, or a homolog thereof being depicted in Table I, application no. 16, column 8, in the same line as SEQ ID NO. 25498, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 25498 is depicted, plastidic is mentioned, or a mitochondrial transit peptide in case in Table I, application no. 16, in column 6 in the same line as SEQ ID NO. 25498 is depicted, mitochondric is mentioned; which is operable linked, too.

In another embodiment thereof the expression cassette does not comprise an additional transit peptide in case in Table I, application no. 16, in column 6 in the same line as SEQ ID NO. 25498 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 5493, or a homolog or fragment thereof, or a homolog thereof being depicted in Table I, application no. 16, column 8, in the same line as SEQ ID NO. 5493, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 5493 is depicted, plastidic is mentioned, or a mitochondrial transit peptide in case in Table I, application no. 16, in column 6 in the same line as SEQ ID NO. 5493 is depicted, mitochondric is mentioned; which is operable linked, too.

In another embodiment thereof the expression cassette does not comprise an additional transit peptide in case in Table I, application no. 16, in column 6 in the same line as SEQ ID NO. 5493 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 29286, or a homolog or fragment thereof, or a homolog thereof being depicted in Table I, application no. 16, column 8, in the same line as SEQ ID NO. 29286, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 29286 is depicted, plastidic is mentioned, or a mitochondrial transit peptide in case in Table I, application no. 16, in column 6 in the same line as SEQ ID NO. 29286 is depicted, mitochondric is mentioned; which is operable linked, too.

In another embodiment thereof the expression cassette does not comprise an additional transit peptide in case in Table I, application no. 16, in column 6 in the same line as SEQ ID NO. 29286 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 31026, or a homolog or fragment thereof, or a homolog thereof being depicted in Table I, application no. 16, column 8, in the same line as SEQ ID NO. 31026, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 31026 is depicted, plastidic is mentioned, or a mitochondrial transit peptide in case in Table I, application no. 16, in column 6 in the same line as SEQ ID NO. 31026 is depicted, mitochondric is mentioned; which is operable linked, too.

In another embodiment thereof the expression cassette does not comprise an additional transit peptide in case in Table I, application no. 16, in column 6 in the same line as SEQ ID NO. 31026 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 114231, or a homolog or fragment thereof, or a homolog thereof being depicted in Table I, application no. 16, column 8, in the same line as SEQ ID NO. 114231, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 114231 is depicted, plastidic is mentioned, or a mitochondrial transit peptide in case in Table I, application no. 16, in column 6 in the same line as SEQ ID NO. 114231 is depicted, mitochondric is mentioned; which is operable linked, too.

In another embodiment thereof the expression cassette does not comprise an additional transit peptide in case in Table I, application no. 16, in column 6 in the same line as SEQ ID NO. 114231 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 73038, or a homolog or fragment thereof, or a homolog thereof being depicted in Table I, application no. 16, column 8, in the same line as SEQ ID NO. 73038, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 73038 is depicted, plastidic is mentioned, or a mitochondrial transit peptide in case in Table I, application no. 16, in column 6 in the same line as SEQ ID NO. 73038 is depicted, mitochondric is mentioned; which is operable linked, too.

In another embodiment thereof the expression cassette does not comprise an additional transit peptide in case in Table I, application no. 16, in column 6 in the same line as SEQ ID NO. 73038 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 102941, or a homolog or fragment thereof, or a homolog thereof being depicted in Table I, application no. 16, column 8, in the same line as SEQ ID NO. 102941, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 102941 is depicted, plastidic is mentioned, or a mitochondrial transit peptide in case in Table I, application no. 16, in column 6 in the same line as SEQ ID NO. 102941 is depicted, mitochondric is mentioned; which is operable linked, too.

In another embodiment thereof the expression cassette does not comprise an additional transit peptide in case in Table I, application no. 16, in column 6 in the same line as SEQ ID NO. 102941 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6510, or a homolog or fragment thereof, or a homolog thereof being depicted in Table I, application no. 16, column 8, in the same line as SEQ ID NO. 6510, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 6510 is depicted, plastidic is mentioned, or a mitochondrial transit peptide in case in Table I, application no. 16, in column 6 in the same line as SEQ ID NO. 6510 is depicted, mitochondric is mentioned; which is operable linked, too.

In another embodiment thereof the expression cassette does not comprise an additional transit peptide in case in Table I, application no. 16, in column 6 in the same line as SEQ ID NO. 6510 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 34301, or a homolog or fragment thereof, or a homolog thereof being depicted in Table I, application no. 16, column 8, in the same line as SEQ ID NO. 34301, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 34301 is depicted, plastidic is mentioned, or a mitochondrial transit peptide in case in Table I, application no. 16, in column 6 in the same line as SEQ ID NO. 34301 is depicted, mitochondric is mentioned; which is operable linked, too.

In another embodiment thereof the expression cassette does not comprise an additional transit peptide in case in Table I, application no. 16, in column 6 in the same line as SEQ ID NO. 34301 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 34602, or a homolog or fragment thereof, or a homolog thereof being depicted in Table I, application no. 16, column 8, in the same line as SEQ ID NO. 34602, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 34602 is depicted, plastidic is mentioned, or a mitochondrial transit peptide in case in Table I, application no. 16, in column 6 in the same line as SEQ ID NO. 34602 is depicted, mitochondric is mentioned; which is operable linked, too.

In another embodiment thereof the expression cassette does not comprise an additional transit peptide in case in Table I, application no. 16, in column 6 in the same line as SEQ ID NO. 34602 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 34889, or a homolog or fragment thereof, or a homolog thereof being depicted in Table I, application no. 16, column 8, in the same line as SEQ ID NO. 34889, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 34889 is depicted, plastidic is mentioned, or a mitochondrial transit peptide in case in Table I, application no. 16, in column 6 in the same line as SEQ ID NO. 34889 is depicted, mitochondric is mentioned; which is operable linked, too.

In another embodiment thereof the expression cassette does not comprise an additional transit peptide in case in Table I, application no. 16, in column 6 in the same line as SEQ ID NO. 34889 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 137834, or a homolog or fragment thereof, or a homolog thereof being depicted in Table I, application no. 16, column 8, in the same line as SEQ ID NO. 137834, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 137834 is depicted, plastidic is mentioned, or a mitochondrial transit peptide in case in Table I, application no. 16, in column 6 in the same line as SEQ ID NO. 137834 is depicted, mitochondric is mentioned; which is operable linked, too.

In another embodiment thereof the expression cassette does not comprise an additional transit peptide in case in Table I, application no. 16, in column 6 in the same line as SEQ ID NO. 137834 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7081, or a homolog or fragment thereof, or a homolog thereof being depicted in Table I, application no. 16, column 8, in the same line as SEQ ID NO. 7081, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 7081 is depicted, plastidic is mentioned, or a mitochondrial transit peptide in case in Table I, application no. 16, in column 6 in the same line as SEQ ID NO. 7081 is depicted, mitochondric is mentioned; which is operable linked, too.

In another embodiment thereof the expression cassette does not comprise an additional transit peptide in case in Table I, application no. 16, in column 6 in the same line as SEQ ID NO. 7081 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 35733, or a homolog or fragment thereof, or a homolog thereof being depicted in Table I, application no. 16, column 8, in the same line as SEQ ID NO. 35733, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 35733 is depicted, plastidic is mentioned, or a mitochondrial transit peptide in case in Table I, application no. 16, in column 6 in the same line as SEQ ID NO. 35733 is depicted, mitochondric is mentioned; which is operable linked, too.

In another embodiment thereof the expression cassette does not comprise an additional transit peptide in case in Table I, application no. 16, in column 6 in the same line as SEQ ID NO. 35733 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7333, or a homolog or fragment thereof, or a homolog thereof being depicted in Table I, application no. 16, column 8, in the same line as SEQ ID NO. 7333, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 7333 is depicted, plastidic is mentioned, or a mitochondrial transit peptide in case in Table I, application no. 16, in column 6 in the same line as SEQ ID NO. 7333 is depicted, mitochondric is mentioned; which is operable linked, too.

In another embodiment thereof the expression cassette does not comprise an additional transit peptide in case in Table I, application no. 16, in column 6 in the same line as SEQ ID NO. 7333 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 36114, or a homolog or fragment thereof, or a homolog thereof being depicted in Table I, application no. 16, column 8, in the same line as SEQ ID NO. 36114, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 36114 is depicted, plastidic is mentioned, or a mitochondrial transit peptide in case in Table I, application no. 16, in column 6 in the same line as SEQ ID NO. 36114 is depicted, mitochondric is mentioned; which is operable linked, too.

In another embodiment thereof the expression cassette does not comprise an additional transit peptide in case in Table I, application no. 16, in column 6 in the same line as SEQ ID NO. 36114 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 36670, or a homolog or fragment thereof, or a homolog thereof being depicted in Table I, application no. 16, column 8, in the same line as SEQ ID NO. 36670, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 36670 is depicted, plastidic is mentioned, or a mitochondrial transit peptide in case in Table I, application no. 16, in column 6 in the same line as SEQ ID NO. 36670 is depicted, mitochondric is mentioned; which is operable linked, too.

In another embodiment thereof the expression cassette does not comprise an additional transit peptide in case in Table I, application no. 16, in column 6 in the same line as SEQ ID NO. 36670 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7941, or a homolog or fragment thereof, or a homolog thereof being depicted in Table I, application no. 16, column 8, in the same line as SEQ ID NO. 7941, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 7941 is depicted, plastidic is mentioned, or a mitochondrial transit peptide in case in Table I, application no. 16, in column 6 in the same line as SEQ ID NO. 7941 is depicted, mitochondric is mentioned; which is operable linked, too.

In another embodiment thereof the expression cassette does not comprise an additional transit peptide in case in Table I, application no. 16, in column 6 in the same line as SEQ ID NO. 7941 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 138256, or a homolog or fragment thereof, or a homolog thereof being depicted in Table I, application no. 16, column 8, in the same line as SEQ ID NO. 138256, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 138256 is depicted, plastidic is mentioned, or a mitochondrial transit peptide in case in Table I, application no. 16, in column 6 in the same line as SEQ ID NO. 138256 is depicted, mitochondric is mentioned; which is operable linked, too.

In another embodiment thereof the expression cassette does not comprise an additional transit peptide in case in Table I, application no. 16, in column 6 in the same line as SEQ ID NO. 138256 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7947, or a homolog or fragment thereof, or a homolog thereof being depicted in Table I, application no. 16, column 8, in the same line as SEQ ID NO. 7947, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 7947 is depicted, plastidic is mentioned, or a mitochondrial transit peptide in case in Table I, application no. 16, in column 6 in the same line as SEQ ID NO. 7947 is depicted, mitochondric is mentioned; which is operable linked, too.

In another embodiment thereof the expression cassette does not comprise an additional transit peptide in case in Table I, application no. 16, in column 6 in the same line as SEQ ID NO. 7947 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38300, or a homolog or fragment thereof, or a homolog thereof being depicted in Table I, application no. 16, column 8, in the same line as SEQ ID NO. 38300, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 38300 is depicted, plastidic is mentioned, or a mitochondrial transit peptide in case in Table I, application no. 16, in column 6 in the same line as SEQ ID NO. 38300 is depicted, mitochondric is mentioned; which is operable linked, too.

In another embodiment thereof the expression cassette does not comprise an additional transit peptide in case in Table I, application no. 16, in column 6 in the same line as SEQ ID NO. 38300 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 8316, or a homolog or fragment thereof, or a homolog thereof being depicted in Table I, application no. 16, column 8, in the same line as SEQ ID NO. 8316, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 8316 is depicted, plastidic is mentioned, or a mitochondrial transit peptide in case in Table I, application no. 16, in column 6 in the same line as SEQ ID NO. 8316 is depicted, mitochondric is mentioned; which is operable linked, too.

In another embodiment thereof the expression cassette does not comprise an additional transit peptide in case in Table I, application no. 16, in column 6 in the same line as SEQ ID NO. 8316 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 8920, or a homolog or fragment thereof, or a homolog thereof being depicted in Table I, application no. 16, column 8, in the same line as SEQ ID NO. 8920, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 8920 is depicted, plastidic is mentioned, or a mitochondrial transit peptide in case in Table I, application no. 16, in column 6 in the same line as SEQ ID NO. 8920 is depicted, mitochondric is mentioned; which is operable linked, too.

In another embodiment thereof the expression cassette does not comprise an additional transit peptide in case in Table I, application no. 16, in column 6 in the same line as SEQ ID NO. 8920 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 112193, or a homolog or fragment thereof, or a homolog thereof being depicted in Table I, application no. 16, column 8, in the same line as SEQ ID NO. 112193, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 112193 is depicted, plastidic is mentioned, or a mitochondrial transit peptide in case in Table I, application no. 16, in column 6 in the same line as SEQ ID NO. 112193 is depicted, mitochondric is mentioned; which is operable linked, too.

In another embodiment thereof the expression cassette does not comprise an additional transit peptide in case in Table I, application no. 16, in column 6 in the same line as SEQ ID NO. 112193 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 39120, or a homolog or fragment thereof, or a homolog thereof being depicted in Table I, application no. 16, column 8, in the same line as SEQ ID NO. 39120, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 39120 is depicted, plastidic is mentioned, or a mitochondrial transit peptide in case in Table I, application no. 16, in column 6 in the same line as SEQ ID NO. 39120 is depicted, mitochondric is mentioned; which is operable linked, too.

In another embodiment thereof the expression cassette does not comprise an additional transit peptide in case in Table I, application no. 16, in column 6 in the same line as SEQ ID NO. 39120 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 99620, or a homolog or fragment thereof, or a homolog thereof being depicted in Table I, application no. 16, column 8, in the same line as SEQ ID NO. 99620, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 99620 is depicted, plastidic is mentioned, or a mitochondrial transit peptide in case in Table I, application no. 16, in column 6 in the same line as SEQ ID NO. 99620 is depicted, mitochondric is mentioned; which is operable linked, too.

In another embodiment thereof the expression cassette does not comprise an additional transit peptide in case in Table I, application no. 16, in column 6 in the same line as SEQ ID NO. 99620 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 138377, or a homolog or fragment thereof, or a homolog thereof being depicted in Table I, application no. 16, column 8, in the same line as SEQ ID NO. 138377, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 138377 is depicted, plastidic is mentioned, or a mitochondrial transit peptide in case in Table I, application no. 16, in column 6 in the same line as SEQ ID NO. 138377 is depicted, mitochondric is mentioned; which is operable linked, too.

In another embodiment thereof the expression cassette does not comprise an additional transit peptide in case in Table I, application no. 16, in column 6 in the same line as SEQ ID NO. 138377 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 80906, or a homolog or fragment thereof, or a homolog thereof being depicted in Table I, application no. 16, column 8, in the same line as SEQ ID NO. 80906, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 80906 is depicted, plastidic is mentioned, or a mitochondrial transit peptide in case in Table I, application no. 16, in column 6 in the same line as SEQ ID NO. 80906 is depicted, mitochondric is mentioned; which is operable linked, too.

In another embodiment thereof the expression cassette does not comprise an additional transit peptide in case in Table I, application no. 16, in column 6 in the same line as SEQ ID NO. 80906 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 81075, or a homolog or fragment thereof, or a homolog thereof being depicted in Table I, application no. 16, column 8, in the same line as SEQ ID NO. 81075, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 81075 is depicted, plastidic is mentioned, or a mitochondrial transit peptide in case in Table I, application no. 16, in column 6 in the same line as SEQ ID NO. 81075 is depicted, mitochondric is mentioned; which is operable linked, too.

In another embodiment thereof the expression cassette does not comprise an additional transit peptide in case in Table I, application no. 16, in column 6 in the same line as SEQ ID NO. 81075 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 81451, or a homolog or fragment thereof, or a homolog thereof being depicted in Table I, application no. 16, column 8, in the same line as SEQ ID NO. 81451, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 81451 is depicted, plastidic is mentioned, or a mitochondrial transit peptide in case in Table I, application no. 16, in column 6 in the same line as SEQ ID NO. 81451 is depicted, mitochondric is mentioned; which is operable linked, too.

In another embodiment thereof the expression cassette does not comprise an additional transit peptide in case in Table I, application no. 16, in column 6 in the same line as SEQ ID NO. 81451 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 81980, or a homolog or fragment thereof, or a homolog thereof being depicted in Table I, application no. 16, column 8, in the same line as SEQ ID NO. 81980, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 81980 is depicted, plastidic is mentioned, or a mitochondrial transit peptide in case in Table I, application no. 16, in column 6 in the same line as SEQ ID NO. 81980 is depicted, mitochondric is mentioned; which is operable linked, too.

In another embodiment thereof the expression cassette does not comprise an additional transit peptide in case in Table I, application no. 16, in column 6 in the same line as SEQ ID NO. 81980 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 42502, or a homolog or fragment thereof, or a homolog thereof being depicted in Table I, application no. 16, column 8, in the same line as SEQ ID NO. 42502, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 42502 is depicted, plastidic is mentioned, or a mitochondrial transit peptide in case in Table I, application no. 16, in column 6 in the same line as SEQ ID NO. 42502 is depicted, mitochondric is mentioned; which is operable linked, too.

In another embodiment thereof the expression cassette does not comprise an additional transit peptide in case in Table I, application no. 16, in column 6 in the same line as SEQ ID NO. 42502 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 138770, or a homolog or fragment thereof, or a homolog thereof being depicted in Table I, application no. 16, column 8, in the same line as SEQ ID NO. 138770, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 138770 is depicted, plastidic is mentioned, or a mitochondrial transit peptide in case in Table I, application no. 16, in column 6 in the same line as SEQ ID NO. 138770 is depicted, mitochondric is mentioned; which is operable linked, too.

In another embodiment thereof the expression cassette does not comprise an additional transit peptide in case in Table I, application no. 16, in column 6 in the same line as SEQ ID NO. 138770 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 10708, or a homolog or fragment thereof, or a homolog thereof being depicted in Table I, application no. 16, column 8, in the same line as SEQ ID NO. 10708, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 10708 is depicted, plastidic is mentioned, or a mitochondrial transit peptide in case in Table I, application no. 16, in column 6 in the same line as SEQ ID NO. 10708 is depicted, mitochondric is mentioned; which is operable linked, too.

In another embodiment thereof the expression cassette does not comprise an additional transit peptide in case in Table I, application no. 16, in column 6 in the same line as SEQ ID NO. 10708 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 44223, or a homolog or fragment thereof, or a homolog thereof being depicted in Table I, application no. 16, column 8, in the same line as SEQ ID NO. 44223, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 44223 is depicted, plastidic is mentioned, or a mitochondrial transit peptide in case in Table I, application no. 16, in column 6 in the same line as SEQ ID NO. 44223 is depicted, mitochondric is mentioned; which is operable linked, too.

In another embodiment thereof the expression cassette does not comprise an additional transit peptide in case in Table I, application no. 16, in column 6 in the same line as SEQ ID NO. 44223 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 138857, or a homolog or fragment thereof, or a homolog thereof being depicted in Table I, application no. 16, column 8, in the same line as SEQ ID NO. 138857, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 138857 is depicted, plastidic is mentioned, or a mitochondrial transit peptide in case in Table I, application no. 16, in column 6 in the same line as SEQ ID NO. 138857 is depicted, mitochondric is mentioned; which is operable linked, too.

In another embodiment thereof the expression cassette does not comprise an additional transit peptide in case in Table I, application no. 16, in column 6 in the same line as SEQ ID NO. 138857 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 10740, or a homolog or fragment thereof, or a homolog thereof being depicted in Table I, application no. 16, column 8, in the same line as SEQ ID NO. 10740, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 10740 is depicted, plastidic is mentioned, or a mitochondrial transit peptide in case in Table I, application no. 16, in column 6 in the same line as SEQ ID NO. 10740 is depicted, mitochondric is mentioned; which is operable linked, too.

In another embodiment thereof the expression cassette does not comprise an additional transit peptide in case in Table I, application no. 16, in column 6 in the same line as SEQ ID NO. 10740 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 45321, or a homolog or fragment thereof, or a homolog thereof being depicted in Table I, application no. 16, column 8, in the same line as SEQ ID NO. 45321, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 45321 is depicted, plastidic is mentioned, or a mitochondrial transit peptide in case in Table I, application no. 16, in column 6 in the same line as SEQ ID NO. 45321 is depicted, mitochondric is mentioned; which is operable linked, too.

In another embodiment thereof the expression cassette does not comprise an additional transit peptide in case in Table I, application no. 16, in column 6 in the same line as SEQ ID NO. 45321 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 45394, or a homolog or fragment thereof, or a homolog thereof being depicted in Table I, application no. 16, column 8, in the same line as SEQ ID NO. 45394, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 45394 is depicted, plastidic is mentioned, or a mitochondrial transit peptide in case in Table I, application no. 16, in column 6 in the same line as SEQ ID NO. 45394 is depicted, mitochondric is mentioned; which is operable linked, too.

In another embodiment thereof the expression cassette does not comprise an additional transit peptide in case in Table I, application no. 16, in column 6 in the same line as SEQ ID NO. 45394 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 46751, or a homolog or fragment thereof, or a homolog thereof being depicted in Table I, application no. 16, column 8, in the same line as SEQ ID NO. 46751, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 46751 is depicted, plastidic is mentioned, or a mitochondrial transit peptide in case in Table I, application no. 16, in column 6 in the same line as SEQ ID NO. 46751 is depicted, mitochondric is mentioned; which is operable linked, too.

In another embodiment thereof the expression cassette does not comprise an additional transit peptide in case in Table I, application no. 16, in column 6 in the same line as SEQ ID NO. 46751 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 84198, or a homolog or fragment thereof, or a homolog thereof being depicted in Table I, application no. 16, column 8, in the same line as SEQ ID NO. 84198, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 84198 is depicted, plastidic is mentioned, or a mitochondrial transit peptide in case in Table I, application no. 16, in column 6 in the same line as SEQ ID NO. 84198 is depicted, mitochondric is mentioned; which is operable linked, too.

In another embodiment thereof the expression cassette does not comprise an additional transit peptide in case in Table I, application no. 16, in column 6 in the same line as SEQ ID NO. 84198 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 49143, or a homolog or fragment thereof, or a homolog thereof being depicted in Table I, application no. 16, column 8, in the same line as SEQ ID NO. 49143, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 49143 is depicted, plastidic is mentioned, or a mitochondrial transit peptide in case in Table I, application no. 16, in column 6 in the same line as SEQ ID NO. 49143 is depicted, mitochondric is mentioned; which is operable linked, too.

In another embodiment thereof the expression cassette does not comprise an additional transit peptide in case in Table I, application no. 16, in column 6 in the same line as SEQ ID NO. 49143 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 84869, or a homolog or fragment thereof, or a homolog thereof being depicted in Table I, application no. 16, column 8, in the same line as SEQ ID NO. 84869, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 84869 is depicted, plastidic is mentioned, or a mitochondrial transit peptide in case in Table I, application no. 16, in column 6 in the same line as SEQ ID NO. 84869 is depicted, mitochondric is mentioned; which is operable linked, too.

In another embodiment thereof the expression cassette does not comprise an additional transit peptide in case in Table I, application no. 16, in column 6 in the same line as SEQ ID NO. 84869 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 55379, or a homolog or fragment thereof, or a homolog thereof being depicted in Table I, application no. 16, column 8, in the same line as SEQ ID NO. 55379, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 55379 is depicted, plastidic is mentioned, or a mitochondrial transit peptide in case in Table I, application no. 16, in column 6 in the same line as SEQ ID NO. 55379 is depicted, mitochondric is mentioned; which is operable linked, too.

In another embodiment thereof the expression cassette does not comprise an additional transit peptide in case in Table I, application no. 16, in column 6 in the same line as SEQ ID NO. 55379 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 55385, or a homolog or fragment thereof, or a homolog thereof being depicted in Table I, application no. 16, column 8, in the same line as SEQ ID NO. 55385, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 55385 is depicted, plastidic is mentioned, or a mitochondrial transit peptide in case in Table I, application no. 16, in column 6 in the same line as SEQ ID NO. 55385 is depicted, mitochondric is mentioned; which is operable linked, too.

In another embodiment thereof the expression cassette does not comprise an additional transit peptide in case in Table I, application no. 16, in column 6 in the same line as SEQ ID NO. 55385 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 11990, or a homolog or fragment thereof, or a homolog thereof being depicted in Table I, application no. 16, column 8, in the same line as SEQ ID NO. 11990, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 11990 is depicted, plastidic is mentioned, or a mitochondrial transit peptide in case in Table I, application no. 16, in column 6 in the same line as SEQ ID NO. 11990 is depicted, mitochondric is mentioned; which is operable linked, too.

In another embodiment thereof the expression cassette does not comprise an additional transit peptide in case in Table I, application no. 16, in column 6 in the same line as SEQ ID NO. 11990 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 87397, or a homolog or fragment thereof, or a homolog thereof being depicted in Table I, application no. 16, column 8, in the same line as SEQ ID NO. 87397, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 87397 is depicted, plastidic is mentioned, or a mitochondrial transit peptide in case in Table I, application no. 16, in column 6 in the same line as SEQ ID NO. 87397 is depicted, mitochondric is mentioned; which is operable linked, too.

In another embodiment thereof the expression cassette does not comprise an additional transit peptide in case in Table I, application no. 16, in column 6 in the same line as SEQ ID NO. 87397 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 119222, or a homolog or fragment thereof, or a homolog thereof being depicted in Table I, application no. 16, column 8, in the same line as SEQ ID NO. 119222, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 119222 is depicted, plastidic is mentioned, or a mitochondrial transit peptide in case in Table I, application no. 16, in column 6 in the same line as SEQ ID NO. 119222 is depicted, mitochondric is mentioned; which is operable linked, too.

In another embodiment thereof the expression cassette does not comprise an additional transit peptide in case in Table I, application no. 16, in column 6 in the same line as SEQ ID NO. 119222 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 101776, or a homolog or fragment thereof, or a homolog thereof being depicted in Table I, application no. 16, column 8, in the same line as SEQ ID NO. 101776, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 101776 is depicted, plastidic is mentioned, or a mitochondrial transit peptide in case in Table I, application no. 16, in column 6 in the same line as SEQ ID NO. 101776 is depicted, mitochondric is mentioned; which is operable linked, too.

In another embodiment thereof the expression cassette does not comprise an additional transit peptide in case in Table I, application no. 16, in column 6 in the same line as SEQ ID NO. 101776 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 116713, or a homolog or fragment thereof, or a homolog thereof being depicted in Table I, application no. 16, column 8, in the same line as SEQ ID NO. 116713, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 116713 is depicted, plastidic is mentioned, or a mitochondrial transit peptide in case in Table I, application no. 16, in column 6 in the same line as SEQ ID NO. 116713 is depicted, mitochondric is mentioned; which is operable linked, too.

In another embodiment thereof the expression cassette does not comprise an additional transit peptide in case in Table I, application no. 16, in column 6 in the same line as SEQ ID NO. 116713 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 58731, or a homolog or fragment thereof, or a homolog thereof being depicted in Table I, application no. 16, column 8, in the same line as SEQ ID NO. 58731, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 58731 is depicted, plastidic is mentioned, or a mitochondrial transit peptide in case in Table I, application no. 16, in column 6 in the same line as SEQ ID NO. 58731 is depicted, mitochondric is mentioned; which is operable linked, too.

In another embodiment thereof the expression cassette does not comprise an additional transit peptide in case in Table I, application no. 16, in column 6 in the same line as SEQ ID NO. 58731 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 58751, or a homolog or fragment thereof, or a homolog thereof being depicted in Table I, application no. 16, column 8, in the same line as SEQ ID NO. 58751, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 58751 is depicted, plastidic is mentioned, or a mitochondrial transit peptide in case in Table I, application no. 16, in column 6 in the same line as SEQ ID NO. 58751 is depicted, mitochondric is mentioned; which is operable linked, too.

In another embodiment thereof the expression cassette does not comprise an additional transit peptide in case in Table I, application no. 16, in column 6 in the same line as SEQ ID NO. 58751 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 58823, or a homolog or fragment thereof, or a homolog thereof being depicted in Table I, application no. 16, column 8, in the same line as SEQ ID NO. 58823, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 58823 is depicted, plastidic is mentioned, or a mitochondrial transit peptide in case in Table I, application no. 16, in column 6 in the same line as SEQ ID NO. 58823 is depicted, mitochondric is mentioned; which is operable linked, too.

In another embodiment thereof the expression cassette does not comprise an additional transit peptide in case in Table I, application no. 16, in column 6 in the same line as SEQ ID NO. 58823 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12698, or a homolog or fragment thereof, or a homolog thereof being depicted in Table I, application no. 16, column 8, in the same line as SEQ ID NO. 12698, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 12698 is depicted, plastidic is mentioned, or a mitochondrial transit peptide in case in Table I, application no. 16, in column 6 in the same line as SEQ ID NO. 12698 is depicted, mitochondric is mentioned; which is operable linked, too.

In another embodiment thereof the expression cassette does not comprise an additional transit peptide in case in Table I, application no. 16, in column 6 in the same line as SEQ ID NO. 12698 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 61070, or a homolog or fragment thereof, or a homolog thereof being depicted in Table I, application no. 16, column 8, in the same line as SEQ ID NO. 61070, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 61070 is depicted, plastidic is mentioned, or a mitochondrial transit peptide in case in Table I, application no. 16, in column 6 in the same line as SEQ ID NO. 61070 is depicted, mitochondric is mentioned; which is operable linked, too.

In another embodiment thereof the expression cassette does not comprise an additional transit peptide in case in Table I, application no. 16, in column 6 in the same line as SEQ ID NO. 61070 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 61723, or a homolog or fragment thereof, or a homolog thereof being depicted in Table I, application no. 16, column 8, in the same line as SEQ ID NO. 61723, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 61723 is depicted, plastidic is mentioned, or a mitochondrial transit peptide in case in Table I, application no. 16, in column 6 in the same line as SEQ ID NO. 61723 is depicted, mitochondric is mentioned; which is operable linked, too.

In another embodiment thereof the expression cassette does not comprise an additional transit peptide in case in Table I, application no. 16, in column 6 in the same line as SEQ ID NO. 61723 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 62079, or a homolog or fragment thereof, or a homolog thereof being depicted in Table I, application no. 16, column 8, in the same line as SEQ ID NO. 62079, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 62079 is depicted, plastidic is mentioned, or a mitochondrial transit peptide in case in Table I, application no. 16, in column 6 in the same line as SEQ ID NO. 62079 is depicted, mitochondric is mentioned; which is operable linked, too.

In another embodiment thereof the expression cassette does not comprise an additional transit peptide in case in Table I, application no. 16, in column 6 in the same line as SEQ ID NO. 62079 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 62717, or a homolog or fragment thereof, or a homolog thereof being depicted in Table I, application no. 16, column 8, in the same line as SEQ ID NO. 62717, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 62717 is depicted, plastidic is mentioned, or a mitochondrial transit peptide in case in Table I, application no. 16, in column 6 in the same line as SEQ ID NO. 62717 is depicted, mitochondric is mentioned; which is operable linked, too.

In another embodiment thereof the expression cassette does not comprise an additional transit peptide in case in Table I, application no. 16, in column 6 in the same line as SEQ ID NO. 62717 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 96980, or a homolog or fragment thereof, or a homolog thereof being depicted in Table I, application no. 16, column 8, in the same line as SEQ ID NO. 96980, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 96980 is depicted, plastidic is mentioned, or a mitochondrial transit peptide in case in Table I, application no. 16, in column 6 in the same line as SEQ ID NO. 96980 is depicted, mitochondric is mentioned; which is operable linked, too.

In another embodiment thereof the expression cassette does not comprise an additional transit peptide in case in Table I, application no. 16, in column 6 in the same line as SEQ ID NO. 96980 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 14275, or a homolog or fragment thereof, or a homolog thereof being depicted in Table I, application no. 16, column 8, in the same line as SEQ ID NO. 14275, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 14275 is depicted, plastidic is mentioned, or a mitochondrial transit peptide in case in Table I, application no. 16, in column 6 in the same line as SEQ ID NO. 14275 is depicted, mitochondric is mentioned; which is operable linked, too.

In another embodiment thereof the expression cassette does not comprise an additional transit peptide in case in Table I, application no. 16, in column 6 in the same line as SEQ ID NO. 14275 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 130903, or a homolog or fragment thereof, or a homolog thereof being depicted in Table I, application no. 16, column 8, in the same line as SEQ ID NO. 130903, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 130903 is depicted, plastidic is mentioned, or a mitochondrial transit peptide in case in Table I, application no. 16, in column 6 in the same line as SEQ ID NO. 130903 is depicted, mitochondric is mentioned; which is operable linked, too.

In another embodiment thereof the expression cassette does not comprise an additional transit peptide in case in Table I, application no. 16, in column 6 in the same line as SEQ ID NO. 130903 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 102061, or a homolog or fragment thereof, or a homolog thereof being depicted in Table I, application no. 16, column 8, in the same line as SEQ ID NO. 102061, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 102061 is depicted, plastidic is mentioned, or a mitochondrial transit peptide in case in Table I, application no. 16, in column 6 in the same line as SEQ ID NO. 102061 is depicted, mitochondric is mentioned; which is operable linked, too.

In another embodiment thereof the expression cassette does not comprise an additional transit peptide in case in Table I, application no. 16, in column 6 in the same line as SEQ ID NO. 102061 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 139173, or a homolog or fragment thereof, or a homolog thereof being depicted in Table I, application no. 16, column 8, in the same line as SEQ ID NO. 139173, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 139173 is depicted, plastidic is mentioned, or a mitochondrial transit peptide in case in Table I, application no. 16, in column 6 in the same line as SEQ ID NO. 139173 is depicted, mitochondric is mentioned; which is operable linked, too.

In another embodiment thereof the expression cassette does not comprise an additional transit peptide in case in Table I, application no. 16, in column 6 in the same line as SEQ ID NO. 139173 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 92540, or a homolog or fragment thereof, or a homolog thereof being depicted in Table I, application no. 16, column 8, in the same line as SEQ ID NO. 92540, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 92540 is depicted, plastidic is mentioned, or a mitochondrial transit peptide in case in Table I, application no. 16, in column 6 in the same line as SEQ ID NO. 92540 is depicted, mitochondric is mentioned; which is operable linked, too.

In another embodiment thereof the expression cassette does not comprise an additional transit peptide in case in Table I, application no. 16, in column 6 in the same line as SEQ ID NO. 92540 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 140110, or a homolog or fragment thereof, or a homolog thereof being depicted in Table I, application no. 16, column 8, in the same line as SEQ ID NO. 140110, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 140110 is depicted, plastidic is mentioned, or a mitochondrial transit peptide in case in Table I, application no. 16, in column 6 in the same line as SEQ ID NO. 140110 is depicted, mitochondric is mentioned; which is operable linked, too.

In another embodiment thereof the expression cassette does not comprise an additional transit peptide in case in Table I, application no. 16, in column 6 in the same line as SEQ ID NO. 140110 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68132, or a homolog or fragment thereof, or a homolog thereof being depicted in Table I, application no. 16, column 8, in the same line as SEQ ID NO. 68132, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 16, in column 6 in the same line as SEQ ID NO. 68132 is depicted, plastidic is mentioned, or a mitochondrial transit peptide in case in Table I, application no. 16, in column 6 in the same line as SEQ ID NO. 68132 is depicted, mitochondric is mentioned; which is operable linked, too.

In another embodiment thereof the expression cassette does not comprise an additional transit peptide in case in Table I, application no. 16, in column 6 in the same line as SEQ ID NO. 68132 is depicted, non-targeted is mentioned.

[0165.1.7.16] to [0170.1.7.16] for the disclosure of theses paragraphs see [0165.1.7.7] to [0170.1.7.7] above

The invention further provides a vector, comprising a nucleic acid molecule comprising 1) a nucleic acid molecule selected from the group consisting of:
(a) a nucleic acid molecule encoding a polypeptide shown in Table II, column 5 or 8, application no. 16, preferably shown in Table II A, application no. 16, in column 5, or in Table II A, application no. 16, column 8, or in Table II B, application no. 16, column 8, or a homolog or a fragment thereof;
(b) a nucleic acid molecule shown in Table I, application no. 16, column 5 or 8, preferably shown in Table I A, application no. 16, in column 5, or in Table I A, application no. 16, column 8, or in Table I B, application no. 16, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 16, preferably shown in Table II A, application no. 16, in column 5, or in Table II A, application no. 16, column 8, or in Table II B, application no. 16, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 16, preferably shown in Table I A, application no. 16, in column 5, or in Table I A, application no. 16, column 8, or in Table I B, application no. 16, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 16;
(i) a nucleic acid molecule encoding a polypeptide having the activity represented by a protein as depicted in column 5 of Table II, application no. 16;
(j) a nucleic acid molecule comprising a polynucleotide which is obtained by amplifying a cDNA library or a genomic library using the primers in Table III, application no. 16, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 16.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wildtype non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 16, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 16.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 16, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 16, in the same line as of the respective nucleic acid molecule according to 1) “plastidic” is depicted, or encoding a mitochondrial transit peptide, especially in case in column 6 of Table I, application no. 16, in the same line as of the respective nucleic acid molecule according to 1) “mitochondric” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 16, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 16, in the same line as of the respective nucleic acid molecule according to 1) “plastidic” is depicted, or encoding a mitochondrial transit peptide, especially in case in column 6 of Table I, application no. 16, in the same line as of the respective nucleic acid molecule according to 1) “mitochondric” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operable linked; a nucleic acid molecule encoding a transit peptide, especially in case in column 6 of Table I, application no. 16, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 16, in the same line as of the respective nucleic acid molecule according to 1) “plastidic” is depicted, or encoding a mitochondrial transit peptide, especially in case in column 6 of Table I, application no. 16, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 16 (in the same line).

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 16, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 16.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 16, in the same line as of the respective nucleic acid molecule according to 1) “plastidic” is depicted, or encoding a mitochondrial transit peptide, especially in case in column 6 of Table I, application no. 16, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 16, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 16, in the same line as of the respective nucleic acid molecule according to 1) “plastidic” is depicted, or encoding a mitochondrial transit peptide, especially in case in column 6 of Table I, application no. 16, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 16 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 16, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 16 (in the same line) and confers a generation or an increase of the respective fine chemical in a non-human organism or a part thereof as compared to a corresponding wild-type non-human organism or part thereof.

In a preferred embodiment of said vector said nucleic acid molecule according to 1 (a), (b), (c), (d), (e), (f), (g), (h), (i), (j) or (k), said promoter and optionally said nucleic acid molecule encoding a transit peptide are encompassed in an expression cassette. Accordingly in an embodiment the invention provides a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

The invention further provides a vector comprising a nucleic acid molecule comprising 1) a nucleic acid molecule selected from the group consisting of:
(a) a nucleic acid molecule encoding a polypeptide shown in Table II, column 5 or 8, application no. 16, preferably shown in Table II A, application no. 16, in column 5, or in Table II A, application no. 16, column 8, or in Table II B, application no. 16, column 8, or a homolog or a fragment thereof;
(b) a nucleic acid molecule shown in Table I, application no. 16, column 5 or 8, preferably shown in Table I A, application no. 16, in column 5, or in Table I A, application no. 16, column 8, or in Table I B, application no. 16, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 16, preferably shown in Table II A, application no. 16, in column 5, or in Table II A, application no. 16, column 8, or in Table II B, application no. 16, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 16, preferably shown in Table I A, application no. 16, in column 5, or in Table I A, application no. 16, column 8, or in Table I B, application no. 16, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 16;
(i) a nucleic acid molecule encoding a polypeptide having the activity represented by a protein as depicted in column 5 of Table II, application no. 16;
(j) a nucleic acid molecule comprising a polynucleotide which is obtained by amplifying a cDNA library or a genomic library using the primers in Table III, application no. 16, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto;
   whereby, preferably, the nucleic acid molecule according to 1 (a), (b), (c), (d), (e), (f), (g), (h), (i), (j) or (k) distinguishes over the sequence depicted in Table I A and/or I B, application no. 16, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 16, column 5 or 8, or the coding region thereof, respectively. In another embodiment, the nucleic acid molecule of the present invention, preferably the coding region thereof, is 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 16, column 5 or 8, or the coding region thereof, respectively. In a further embodiment the nucleic acid molecule does not encode the polypeptide sequence shown in Table II A and/or II B, application no. 16, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 16, column 5 or 8 and does not encode a protein of the sequence shown in Table II A and/or II B, application no. 16, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 16, column 5 and 8, or the coding region thereof. In a further embodiment, the protein of the present invention is at least 30%,40%, 50%, 60%, 70%, 80%, 90%, 95% identical to protein sequence depicted in Table II A and/or II B, application no. 16, column 5 or 8 but less than 100%, preferably less than 99.999%, 99.99% or 99.9%, more preferably less than 99%, 985, 97%, 96% or 95% identical to the sequence shown in Table II A and/or II B, application no. 16, column 5 or 8.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 16.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wildtype non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 16, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 16.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 16, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 16, in the same line as of the respective nucleic acid molecule according to 1) “plastidic” is depicted, or encoding a mitochondrial transit peptide, especially in case in column 6 of Table I, application no. 16, in the same line as of the respective nucleic acid molecule according to 1) “mitochondric” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 16, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 16, in the same line as of the respective nucleic acid molecule according to 1) “plastidic” is depicted, or encoding a mitochondrial transit peptide, especially in case in column 6 of Table I, application no. 16, in the same line as of the respective nucleic acid molecule according to 1) “mitochondric” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 16, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 16, in the same line as of the respective nucleic acid molecule according to 1) “plastidic” is depicted, or encoding a mitochondrial transit peptide, especially in case in column 6 of Table I, application no. 16, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 16.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 16, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 16.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 16, in the same line as of the respective nucleic acid molecule according to 1) “plastidic” is depicted, or encoding a mitochondrial transit peptide, especially in case in column 6 of Table I, application no. 16, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 16, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 16, in the same line as of the respective nucleic acid molecule according to 1) “plastidic” is depicted, or encoding a mitochondrial transit peptide, especially in case in column 6 of Table I, application no. 16, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 16 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 16, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 16 and confers a generation or an increase of the respective fine chemical in a non-human organism or a part thereof as compared to a corresponding wild-type non-human organism or part thereof.

[0181.1.7.16] to [0209.1.7.16] for the disclosure of these paragraphs see [0181.1.7.7] to [0209.1.7.7] above

The genes of the invention, coding in one embodiment for an activity selected from the group consisting of 2-C-methyl-D-erythritol 4-phosphate cytidylyltransferase, acetolactate synthase III large subunit, acetyltransferase, acid shock protein, amino acid ABC transporter permease protein, amino acid acetyltransferase, At1g47380-protein, ATP synthase subunit E, ATP-binding component of a transport system, auxin response factor, AX653549-protein, AY087308-protein, b1003-protein, b1097-protein, b1522-protein, b2032-protein, b2739-protein, b2849-protein, b3817-protein, b4029-protein, beta-galactosidase, calcium-dependent protein kinase, cation-transporting ATPase, CCAAT-binding transcription factor, cell division control protein, colanic acid biosynthesis protein, CTP synthase, cyclin D , disease resistance protein, enoyl-CoA hydratase, fatty acid desaturase, F-box protein, flavodoxin, gluconate transport system permease 3, glutamine amidotransferase, glutaredoxin, glycerol dehydrogenase, glycerol-3-phosphate dehydrogenase, glycosyl transferase, halotolerance protein, heat shock protein Hsp40, heat shock transcription factor, histone acetyltransferase, hydrolase, isopropylmalate isomerase large subunit, L-aspartate oxidase, lipase, MADS box protein transcription factor, malate dehydrogenase, methylglyoxal synthase, methyltransferase, mitogen-activated protein kinase, monothiol glutaredoxin, multiple antibiotic resistance protein, N-acetyl-gamma-glutamylphosphate reductase, NADH dehydrogenase I chain I, oxidoreductase, permease protein of ABC transporter, phenylalanine ammonia-lyase, phosphoadenosine phosphosulfate reductase, phosphopantothenoylcysteine decarboxylase, photosystem II protein, protein kinase, protein phosphatase, pyruvate kinase, recombinase A, Ribose import ATP-binding protein, Sec-independent protein translocase subunit, serine protease, serine/threonine-protein phosphatase, sll1761-protein, slr1107-protein, sterol desaturase, threonine synthase, transcription factor, transcriptional regulator, TTC1386-protein, uroporphyrin-III C-methyltransferase, ycl027w-protein, zinc finger protein, and zinc transporter are also called “FCRP genes”.

[0211.1.7.16] to [0225.1.7.16] for the disclosure of these paragraphs see [0211.1.7.7] to [0225.1.7.7] above

In addition to the sequence mentioned in Table I, application no. 16, column 5 or 8, preferably the coding region thereof, or its homologs or fragments, it may be advantageous additionally to express and/or mutate further genes in the non-human organisms. Especially advantageously, additionally at least one further gene of the biosynthetic pathway of the fine chemical is expressed in the non-human organisms such as plants or microorganisms. It is also possible that the regulation of the natural genes has been modified advantageously so that the gene and/or its gene product is no longer subject to the regulatory mechanisms which exist in the non-human organisms. This leads to an increased synthesis of the respective cofactor since, for example, feedback regulations no longer exist to the same extent or not at all. In addition it might be advantageously to combine the nucleic acids sequences of the invention containing the sequences shown in the respective line in Table I, application no. 16, column 5 or 8, preferably the coding region thereof, or homologs or fragments thereof, with genes which generally support or enhance the growth or yield of the target non-human organisms, for example genes which lead to faster growth rate of non human organism like microorganisms or plants or genes which produce stress-, pathogen-, or herbicide-resistant plants.

In a further embodiment of the process of the invention, non-human organisms are grown, in which there is in addition to the expression of the nucleic acid molecule according to the invention simultaneous expression of at least one nucleic acid molecule or one of the genes which code for proteins involved in the isoprenoid and/or quinone metabolism, in particular in Coenzyme Q9 or Coenzyme Q10 synthesis. Especially advantageously, additionally at least one further gene of the isoprenoid biosynthetic pathway such as for acetyl CoA, HMG-CoA, Mevalonate, Isopentyl pyrophosphate, Geranyl pyrophosphate, Farnesyl pyrophosphate e.g. HMG-CoA Reductase, Mevalonate, Kinase, etc., is expressed in the organisms such as plants or microorganisms. It is also possible that the regulation of the natural genes has been modified advantageously so that the gene and/or its gene product is no longer subject to the regulatory mechanisms which exist in the organisms.

This leads to an increased synthesis of the isoprenoids, coenzyme precursor or coenzymes, preferably Q9 and/or Q10, as desired since, for example, feedback regulations no longer exist to the same extent or not at all.

In a further embodiment of the process of the invention, non-human organisms are grown, in which there is in addition to the expression of the nucleic acid molecule according to the invention simultaneous expression of at least one nucleic acid molecule or one of the genes which code for proteins involved in the tryptophan metabolism and/or involved in the pyridine nucleotide cycling: nicotinate phosphoribosyltransferase (EC 2.4.2.11) and/or nicotinamidas (EC 3.5.1.19).

In a further embodiment of the process of the invention, non-human organisms are grown, in which there is in addition to the expression of the nucleic acid molecule according to the invention simultaneous expression of at least one nucleic acid molecule or one of the genes which code for proteins involved in the octanoic acid metabolism, for example LipA (EC 2.8.1.8) and/or LipB (EC 2.3.1.181) and/or genes which code for proteins having activity in conversion of octanyolated protein domains to lipoylated protein domains using S-adenosyl-methionine as sulfur donor have been identified, e.g.: lipoic acid.

[0228.1.7.16] to [0239.1.7.16] for the disclosure of these paragraphs see [0228.1.7.7] to [0239.1.7.7] above

In an embodiment of the present invention the nucleic acid construct of the present invention comprises a nucleic acid molecule as depicted in the respective line in Table I, application no. 16, column 5 or 8, preferably the coding region thereof, a fragment or a homolog thereof, and a plant promoter, such as the Big 35S promoter, the DC3 promoter, the PC Ubi promoter, the LegB4-promoter, the Super promoter, the USP promoter or the like. In another embodiment the nucleic acid construct of the present invention comprises a nucleic acid molecule as depicted in the respective line in Table I, application no. 16, column 5 or 8, preferably the coding region thereof, a fragment or a homolog thereof, and a plant promoter such as, for example, the USP, the LegB4-, the DC3 promoter or the ubiquitin promoter from parsley or other herein mentioned promoters. In a further embodiment the nucleic acid construct of the present invention comprises a nucleic acid molecule as depicted in the respective line in Table I, application no. 16, column 5 or 8, preferably the coding region thereof, a fragment or a homolog thereof, and a plant promoter, such as the Big 35S promoter, the PC Ubi promoter, the Super promoter, the USP promoter or the like. In addition different terminators may advantageously be used in these nucleic acid constructs; examples for transcriptional termination are polyadenylation signals.

[0241.1.7.16] to [0245.1.7.16] for the disclosure of these paragraphs see [0241.1.7.7] to [0245.1.7.7] above

Other preferred sequences for use in operable linkage in gene expression constructs are targeting sequences, which are required for targeting the gene product into specific cell compartments (for a review, see Kermode, Crit. Rev. Plant Sci. 15 (4), 285 (1996) and references cited therein), for example into the vacuole, the nucleus, all types of plastids, such as amyloplasts, chloroplasts, chromoplasts, the extracellular space, the mitochondria, the endoplasmic reticulum, elaioplasts, peroxisomes, glycosomes, and other compartments of cells or extracellular; preferred are sequences, which are involved in targeting to plastids as mentioned above. Sequences, which must be mentioned in this context are, in particular, the signal-peptide- or transit-peptide-encoding sequences which are known per se. For example, plastid-transit-peptide-encoding sequences enable the targeting of the expression product into the plastids of a plant cell. Targeting sequences are also known for eukaryotic and to a lower extent for prokaryotic organisms and can advantageously be operable linked with the nucleic acid molecule of the present invention as shown in Table I, application no. 16, columns 5 and 8, preferably the coding region thereof, or homologs or fragments thereof, and described herein to achieve an expression in one of said compartments or extracellular.

[0247.1.7.16] to [0249.1.7.16] for the disclosure of theses paragraphs see [0247.1.7.7] to [0249.1.7.7] above

Preferred promoters are in particular those which bring about gene expression in tissues and organs, such as leaves, roots, seeds, pollen or xylem. Examples of tissue preferred or organ preferred promoters include, but are not limited to fruit-preferred, ovule-preferred, male tissue-preferred, seed-preferred, pollen-preferred integument-preferred, tuber-preferred, stalk-preferred, pericarp-preferred, leaf-preferred, stigma-preferred, pollen-preferred, anther-preferred, petal-preferred, sepal-preferred, pedicel-preferred, silique-preferred, stem-preferred, root-preferred promoters, and the like. Especially those promoters are preferred which bring about gene expression in tissues and organs in which the biosynthesis of cofactors, especially coenzyme Q10, coenzyme Q9, lipoic acid, and/or nicotinic acid takes place, like in seed cells, such as endosperm cells and cells of the developing embryo. Seed promoters are preferentially expressed during seed development and/or germination. For example, seed preferred promoters can be embryo-preferred, endosperm preferred and seed coat-preferred (see Thompson et al., BioEssays 10, 108 (1989)). Examples of seed preferred promoters include, but are not limited to, cellulose synthase (celA), Cim1, gamma-zein, globulin-1, maize 19 kD zein (cZ19B1), and the like. Other suitable promoters are the oilseed rape napin gene promoter (U.S. Pat. No. 5,608,152), the Vicia faba USP promoter (Baeumlein et al., Mol Gen Genet, 225 (3), 459 (1991)), the Arabidopsis oleosin promoter (WO 98/45461), the Phaseolus vulgaris phaseolin promoter (U.S. Pat. No. 5,504,200), the Brassica Bce4 promoter (WO 91/13980), the bean arcs promoter, the carrot DcG3 promoter, or the Legumin B4 promoter (LeB4) (Baeumlein et al., Plant Journal, 2 (2), 233 (1992)), and promoters which bring about the seed-specific expression in monocotyledonous plants such as maize, barley, wheat, rye, rice and the like. Advantageous seedspecific promoters are the sucrose binding protein promoter (WO 00/26388), the phaseolin promoter and the napin promoter. Suitable promoters which must be considered are the barley Ipt2 or Ipt1 gene promoter (WO 95/15389 and WO 95/23230), and the promoters described in WO 99/16890 (promoters from the barley hordein gene, the rice glutelin gene, the rice oryzin gene, the rice prolamin gene, the wheat gliadin gene, the wheat glutelin gene, the maize zein gene, the oat glutelin gene, the sorghum kasirin gene and the rye secalin gene). Further suitable promoters are Amy32b, Amy 6-6 and Aleurain (U.S. Pat. No. 5,677,474), Bce4 (oilseed rape) (U.S. Pat. No. 5,530,149), glycinin (soya) (EP 571 741], -phosphoenolpyruvate carboxylase (soya) (JP 06/62870), ADR12-2 (soya) (WO 98/08962), isocitrate lyase (oilseed rape) (U.S. Pat. No. 5,689,040]) or α-amylase (barley) (EP 781 8499. Other promoters which are available for the expression of genes in plants are leaf-specific promoters such as those described in DE-A 19 644 478 or lightregulated promoters such as, for example, the pea petE promoter.

[0251.1.7.16] to [0266.1.7.16] for the disclosure of these paragraphs see [0251.1.7.7] to [0266.1.7.7] above

An embodiment of the invention relates to a vector where the nucleic acid molecule according to the invention is linked operably to regulatory sequences which permit the expression in a prokaryotic or eukaryotic host. A further embodiment of the invention relates to a vector in which a nucleic acid sequence encoding one of the polypeptides shown in the respective line in Table II, application no. 16, columns 5 or 8, or homologs or fragments thereof, is functionally linked to a targeting sequence such as a plastidial or mitochondrial targeting sequence.

A further embodiment of the invention relates to a vector in which a nucleic acid sequence encoding one of the polypeptides shown in the respective line in Table II, application no. 16, columns 5 or 8, or homologs or fragments thereof, is functionally linked to (a) regulatory sequence which permit the expression in an organelle, such as plastids or mitochondria, preferably plastids.

A further embodiment of the invention relates to a vector which comprises a nucleic acid sequence encoding one of the polypeptides shown in the respective line in Table II, application no. 16, columns 5 or 8, or homologs or fragments thereof, but no additional targeting sequence.

[0268.1.7.16] to [0273.1.7.16] for the disclosure of these paragraphs see [0268.1.7.7] to [0273.1.7.7] above

A further object of the invention relates to the use of a nucleic acid construct, e.g. an expression cassette, comprising one or more nucleic acid molecules encoding one or more polypeptides as depicted in the respective line(s) in Table II, application no. 16, columns 5 or 8, or homologs or fragments thereof, or comprising one or more nucleic acid molecules as depicted in the respective line(s) in Table I, application no. 16, columns 5 or 8, preferably the coding region thereof, or homologs or fragments thereof, or comprising a nucleic acid molecule hybridizing therewith for the transformation of a microorganism or a plant cell, plant tissues or a part of a plant

In doing so, depending on the choice of promoter, the nucleic acid molecules shown in the respective line in Table I, application no. 16, column 5 or 8, preferably the coding region thereof, or homologs or fragments thereof, or the nucleic acid molecules encoding a polypeptide as depicted in the respective line in Table II, application no. 16, column 5 or 8, or homologs or fragments thereof, can be expressed in the whole plant or specifically e.g. in the leaves, in the seeds, the nodules, in roots, in the stem or other parts of the plant. Those transgenic plants overproducing sequences, e.g. as depicted in the respective line in Table I, application no. 16, column 5 or 8, preferably the coding region thereof, or homologs or fragments thereof, the plant cell, plant tissues, plants, the reproductive material thereof, or parts thereof are a further object of the present invention.

Within the framework of the present invention, the production of the coenzyme Q10, coenzyme Q9, lipoic acid, or nicotinic acid is due to the generation or over-expression of one or more polypeptides as depicted in the respective line(s) in Table II, application no. 16, column 5 or 8, or homologs or fragments thereof, or encoded by the corresponding nucleic acid molecules as depicted in the respective line(s) in Table I, application no. 16, column 5 or 8, preferably the coding region thereof, or homologs or fragments thereof, in the non-human organism according to the invention, advantageously in the transgenic microorganism or plant according to the invention, at least for the duration of at least one plant generation.

A constitutive expression of the polypeptide as depicted in the respective line in Table II, application no. 16, column 5 or 8, or homologs or fragments thereof, or encoded by the respective nucleic acid molecule as depicted in the respective line in Table I, application no. 16, column 5 or 8, preferably the coding region thereof, or homologs or fragments thereof, may be advantageous. On the other hand, an inducible expression may also appear desirable. Expression of the polypeptide sequences of the invention can be either directed to the cytosol or to the organelles, such as plastids or mitochondria, preferably the plastids of the host cells, preferably the plant cells, or non-targeted.

for the disclosure of this paragraph see [0278.1.7.7] above

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 16, column 5 or 8, preferably shown in Table II A, application no. 16, column 5, or in Table II A, application no. 16, column 8, or in Table II B, application no. 16, column 8, or a homolog or a fragment thereof;
   (b) a nucleic acid molecule shown in Table I, application no. 16, column 5 or 8, preferably shown in Table I A, application no. 16, column 5, or in Table I A, application no. 16, column 8, or in Table I B, application no. 16, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 16, preferably shown in Table II A, application no. 16, column 5, or in Table II A, application no. 16, column 8, or in Table II B, application no. 16, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 16, preferably shown in Table I A, application no. 16, column 5, or in Table I A, application no. 16, column 8, or in Table I B, application no. 16, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 16;
   (i) a nucleic acid molecule encoding a polypeptide having the activity represented by a respective protein as depicted in column 5 of Table II, application no. 16;
   (j) a nucleic acid molecule comprising a polynucleotide which is obtained by amplifying a cDNA library or a genomic library using the primers in Table III, application no. 16, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising an expression cassette comprising
[0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 16, column 5 or 8, preferably shown in Table II A, application no. 16, column 5, or in Table II A, application no. 16, column 8, or in Table II B, application no. 16, column 8, or a homolog or a fragment thereof;
   (b) a nucleic acid molecule shown in Table I, application no. 16, column 5 or 8, preferably shown in Table I A, application no. 16, column 5, or in Table I A, application no. 16, column 8, or in Table I B, application no. 16, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 16, preferably shown in Table II A, application no. 16, column 5, or in Table II A, application no. 16, column 8, or in Table II B, application no. 16, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 16, preferably shown in Table I A, application no. 16, column 5, or in Table I A, application no. 16, column 8, or in Table I B, application no. 16, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 16;
   (i) a nucleic acid molecule encoding a polypeptide having the activity represented by a respective protein as depicted in column 5 of Table II, application no. 16;
   (j) a nucleic acid molecule comprising a polynucleotide which is obtained by amplifying a cDNA library or a genomic library using the primers in Table III, application no. 16, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide; which are operable linked;]
   or comprising or being transformed by a vector comprising
[1) a nucleic acid molecule selected from the group consisting of
   (a) a nucleic acid molecule encoding a polypeptide shown in Table II, application no. 16, column 5 or 8, preferably shown in Table II A, application no. 16, column 5, or in Table II A, application no. 16, column 8, or in Table II B, application no. 16, column 8, or a homolog or a fragment thereof;
   (b) a nucleic acid molecule shown in Table I, application no. 16, column 5 or 8, preferably shown in Table I A, application no. 16, column 5, or in Table I A, application no. 16, column 8, or in Table I B, application no. 16, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 16, preferably shown in Table II A, application no. 16, column 5, or in Table II A, application no. 16, column 8, or in Table II B, application no. 16, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 16, preferably shown in Table I A, application no. 16, column 5, or in Table I A, application no. 16, column 8, or in Table I B, application no. 16, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 16;
   (i) a nucleic acid molecule encoding a polypeptide having the activity represented by a respective protein as depicted in column 5 of Table II, application no. 16;
   (j) a nucleic acid molecule comprising a polynucleotide which is obtained by amplifying a cDNA library or a genomic library using the primers in Table III, application no. 16, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 16.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 16 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 16, in the same line as of the respective nucleic acid molecule according to 1) “plastidic” is depicted, or encoding a mitochondrial transit peptide, especially in case in column 6 of Table I, application no. 16, in the same line as of the respective nucleic acid molecule according to 1) “mitochondric” is depicted.

In another embodiment of said transgenic non-human organism the expression cassette does not comprise 2) a nucleic acid molecule encoding a transit peptide, especially in case in column 6 of Table I, application no. 16, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 16.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 16, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 16, in the same line as of the respective nucleic acid molecule according to 1) “plastidic” is depicted, or encoding a mitochondrial transit peptide, especially in case in column 6 of Table I, application no. 16, in the same line as of the respective nucleic acid molecule according to 1) “mitochondric” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 16, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 16, in the same line as of the respective nucleic acid molecule according to 1) “plastidic” is depicted, or encoding a mitochondrial transit peptide, especially in case in column 6 of Table I, application no. 16, in the same line as of the respective nucleic acid molecule according to 1) “mitochondric” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 16, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 16, in the same line as of the respective nucleic acid molecule according to 1) “plastidic” is depicted, or encoding a mitochondrial transit peptide, especially in case in column 6 of Table I, application no. 16, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 16.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 16, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 16.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 16, in the same line as of the respective nucleic acid molecule according to 1) “plastidic” is depicted, or encoding a mitochondrial transit peptide, especially in case in column 6 of Table I, application no. 16, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 16, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 16, in the same line as of the respective nucleic acid molecule according to 1) “plastidic” is depicted, or encoding a mitochondrial transit peptide, especially in case in column 6 of Table I, application no. 16, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 16 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 16, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 16 (in the same line) and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 16, column 5 or 8, preferably shown in Table II A, application no. 16, column 5, or in Table II A, application no. 16, column 8, or in Table II B, application no. 16, column 8, or a homolog or a fragment thereof;
   (b) a nucleic acid molecule shown in Table I, application no. 16, column 5 or 8, preferably shown in Table I A, application no. 16, column 5, or in Table I A, application no. 16, column 8, or in Table I B, application no. 16, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 16, preferably shown in Table II A, application no. 16, column 5, or in Table II A, application no. 16, column 8, or in Table II B, application no. 16, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 16, preferably shown in Table I A, application no. 16, column 5, or in Table I A, application no. 16, column 8, or in Table I B, application no. 16, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 16;
   (i) a nucleic acid molecule encoding a polypeptide having the activity represented by a respective protein as depicted in column 5 of Table II, application no. 16;
   (j) a nucleic acid molecule comprising a polynucleotide which is obtained by amplifying a cDNA library or a genomic library using the primers in Table III, application no. 16, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising an expression cassette comprising
[0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 16, column 5 or 8, preferably shown in Table II A, application no. 16, column 5, or in Table II A, application no. 16, column 8, or in Table II B, application no. 16, column 8, or a homolog or a fragment thereof;
   (b) a nucleic acid molecule shown in Table I, application no. 16, column 5 or 8, preferably shown in Table I A, application no. 16, column 5, or in Table I A, application no. 16, column 8, or in Table I B, application no. 16, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 16, preferably shown in Table II A, application no. 16, column 5, or in Table II A, application no. 16, column 8, or in Table II B, application no. 16, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 16, preferably shown in Table I A, application no. 16, column 5, or in Table I A, application no. 16, column 8, or in Table I B, application no. 16, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 16;
   (i) a nucleic acid molecule encoding a polypeptide having the activity represented by a respective protein as depicted in column 5 of Table II, application no. 16;
   (j) a nucleic acid molecule comprising a polynucleotide which is obtained by amplifying a cDNA library or a genomic library using the primers in Table III, application no. 16, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      and, if desired
2) nucleic acid molecule encoding a transit peptide, preferably encoding a plastidal transit peptide or a mitochondrial transit peptide;
   which are operable linked;]
   or comprising or being transformed by a vector comprising
[1) a nucleic acid molecule selected from the group consisting of
   (a) a nucleic acid molecule encoding a polypeptide shown in Table II, application no. 16, column 5 or 8, preferably shown in Table II A, application no. 16, column 5, or in Table II A, application no. 16, column 8, or in Table II B, application no. 16, column 8, or a homolog or a fragment thereof;
   (b) a nucleic acid molecule shown in Table I, application no. 16, column 5 or 8, preferably shown in Table I A, application no. 16, column 5, or in Table I A, application no. 16, column 8, or in Table I B, application no. 16, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 16, preferably shown in Table II A, application no. 16, column 5, or in Table II A, application no. 16, column 8, or in Table II B, application no. 16, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 16, preferably shown in Table I A, application no. 16, column 5, or in Table I A, application no. 16, column 8, or in Table I B, application no. 16, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 16;
   (i) a nucleic acid molecule encoding a polypeptide having the activity represented by a respective protein as depicted in column 5 of Table II, application no. 16;
   (j) a nucleic acid molecule comprising a polynucleotide which is obtained by amplifying a cDNA library or a genomic library using the primers in Table III, application no. 16, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      whereby, preferably, the nucleic acid molecule according to 1 (a), (b), (c), (d), (e), (f), (g), (h), (i), (j) or (k) distinguishes over the sequence depicted in Table I A and/or I B, application no. 16, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 16, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 16, column 5 or 8, or the coding region thereof, respectively. In a further embodiment the nucleic acid molecule does not encode the polypeptide sequence shown in Table II A and/or II B, application no. 16, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 16, column 5 or 8 and does not encode a protein of the sequence shown in Table II A and/or II B, application no. 16, column 5 or 8. Accordingly, in one embodiment, the protein encoded by a sequence of a nucleic acid according to 1 (a), (b), (c), (d), (e), (f), (g), (h), (i), (j) or (k) does not consist of the sequence shown in Table I A and/or I B, application no. 16, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 16, column 5 or 8 but less than 100%, preferably less than 99.999%, 99.99%, 99.9%, 99%, 98%, 97%, 96% or 95% identical to the sequence shown in Table II A and/or II B, application no. 16, column 5 or 8;
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 16.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 16 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 16, in the same line as of the respective nucleic acid molecule according to 1) “plastidic” is depicted, or encoding a mitochondrial transit peptide, especially in case in column 6 of Table I, application no. 16, in the same line as of the respective nucleic acid molecule according to 1) “mitochondric” is depicted.

In another embodiment thereof the expression cassette does not comprise 2) a nucleic acid molecule encoding a transit peptide, especially in case in column 6 of Table I, application no. 16, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 16.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the same line in column 5 of Table II, application no. 16, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 16, in the same line as of the respective nucleic acid molecule according to 1) “plastidic” is depicted, or encoding a mitochondrial transit peptide, especially in case in column 6 of Table I, application no. 16, in the same line as of the respective nucleic acid molecule according to 1) “mitochondric” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 16, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 16, in the same line as of the respective nucleic acid molecule according to 1) “plastidic” is depicted, or encoding a mitochondrial transit peptide, especially in case in column 6 of Table I, application no. 16, in the same line as of the respective nucleic acid molecule according to 1) “mitochondric” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 16, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 16, in the same line as of the respective nucleic acid molecule according to 1) “plastidic” is depicted, or encoding a mitochondrial transit peptide, especially in case in column 6 of Table I, application no. 16, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 16.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 16, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 16.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 16, in the same line as of the respective nucleic acid molecule according to 1) “plastidic” is depicted, or encoding a mitochondrial transit peptide, especially in case in column 6 of Table I, application no. 16, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 16, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 16, in the same line as of the respective nucleic acid molecule according to 1) “plastidic” is depicted, or encoding a mitochondrial transit peptide, especially in case in column 6 of Table I, application no. 16, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 16 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 16, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 16 and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

[0291.1.7.16] to [0299.1.7.16] for the disclosure of these paragraphs see [0291.1.7.7] to [0299.1.7.7] above

The term “biological active portion” or “biological activity” means a polypeptide as depicted in the respective line in Table II, application no. 16, column 3 or a portion of said polypeptide which still has at least 10% or 20%, preferably 30%, 40%, 50% or 60%, especially preferably 70%, 75%, 80%, 90% or 95% of the enzymatic or biological activity of the natural or starting enzyme or protein.

[0301.1.7.16] to [0304.1.7.16] for the disclosure of these paragraphs see [0301.1.7.7] to [0304.1.7.7] above

Synthetic oligonucleotide primers for the amplification, e.g. as shown in the respective line in Table III, application no. 16, column 8, by means of polymerase chain reaction can be generated on the basis of a sequence shown herein, for example the sequence shown in the respective line in Table I, application no. 16, columns 5 or 8, or the sequences derived from Table II, application no. 16, columns 5 or 8, respectively.

Moreover, it is possible to identify a conserved region of a protein from various organism by carrying out protein sequence alignments with the polypeptide encoded by the nucleic acid molecules of the present invention, in particular with the sequences encoded by the nucleic acid molecule shown in the respective line in column 5 or 8 of Table I, application no. 16, from which conserved regions, and in turn, degenerate primers can be derived. Conserved regions are those, which show a very little variation in the amino acid in one particular position of several homologs from different origin. The consensus sequence and polypeptide motifs shown in the respective line in column 8 of Table IV, application no. 16, are derived from such alignments of homologous proteins.

In an embodiment of the present invention, in the method of the present invention the activity of a polypeptide comprising or consisting of a consensus sequence or at least one polypeptide motif shown in the respective line in Table IV, application no. 16, column 8, is increased. In another embodiment thereof, the method of the present invention relates to a polypeptide comprising or consisting of a consensus sequence shown in the respective line in Table IV, application no. 16, column 8 or comprising at least one polypeptide motif shown in the respective line in Table IV, application no. 16, column 8, whereby not more than 20, 15, 10, 9, 8, 7, 6, 5 4, 3, 2 or 1, or 0 of the amino acid positions indicated can be replaced by any amino acid. In another embodiment thereof, the method of the present invention relates to a polypeptide comprising or consisting of a consensus sequence shown in the respective line in Table IV, application no. 16, column 8 or comprising at least one polypeptide motif shown in the respective line in Table IV, application no. 16, column 8, whereby not more than 15%, 10%, 5%, 4%, 3%, 2% or 1%, or 0% of the amino acid positions defined as distinct specific amino acids are/is replaced by another amino acid. In another embodiment thereof, the method of the present invention relates to a polypeptide comprising or consisting of a consensus sequence shown in the respective line in Table IV, application no. 16, column 8 or comprising at least one polypeptide motif shown in the respective line in Table IV, application no. 16, column 8, whereby not more than 20, 15, 10, 9, 8, 7, 6, 5, 4, 3, 2 or 1, or 0 amino acids are inserted into a consensus sequence or polypeptide motif.

In an embodiment of the present invention, in the method of the present invention the activity of a polypeptide comprising or consisting of a consensus sequence or at least one polypeptide motif shown in the respective line in Table IV, application no. 16, column 8, is increased, whereby said polypeptide distinguishes over the sequence depicted in Table II, application no. 16, columns 5 or 8 by one or more amino acids.

In one embodiment, said polypeptide of the invention distinguishes over the sequence shown in the respective line in Table II, application no. 16, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II B, application no. 16, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in Table II, application no. 16, columns 5 or 8.

[0309.1.7.16] to [0321.1.7.16] for the disclosure of these paragraphs see [0309.1.7.7] to [0321.1.7.7] above

Polypeptides having above-mentioned activity, i.e. conferring the production or the increased production of the fine chemical coenzyme Q10, coenzyme Q9, lipoic acid, or nicotinic acid as compared to a corresponding, e.g. non-transformed, wild type non-human organism, like a microorganism or a plant cell, plant or part thereof, can be encoded by other DNA sequences which hybridize to the sequences shown in the respective line in Table I, application no. 16, columns 5 and 8, preferably the coding region thereof, at least under relaxed hybridization conditions and which encode the expression of polypeptides conferring the production or the increased production of the respective fine chemical coenzyme Q10, coenzyme Q9, lipoic acid, or nicotinic acid as compared to a corresponding, e.g. non-transformed, wild type non-human organism, like a microorganism or a plant cell, plant or part thereof.

[0323.1.7.16] to [0329.1.7.16] for the disclosure of these paragraphs see [0323.1.7.7] to [0329.1.7.7] above

Further, the nucleic acid molecule of the invention comprises a nucleic acid molecule, which is a complement of one of the nucleotide sequences of above-mentioned nucleic acid molecules or a portion thereof. A nucleic acid molecule or its sequence which is complementary to one of the nucleotide molecules or sequences shown in the respective line in Table I, application no. 16, columns 5 or 8, preferably the coding region thereof, homologs or fragments thereof, is one which is sufficiently complementary to one of the nucleotide molecules or sequences shown in the respective line in Table I, application no. 16, columns 5 or 8, preferably the coding region thereof, homologs or fragments thereof, such that it can hybridize to one of the nucleotide sequences shown in Table I, application no. 16, columns 5 or 8, preferably the coding region thereof, homologs or fragments thereof, thereby forming a stable duplex. Preferably, the hybridization is performed under stringent hybridization conditions. However, a complement of one of the herein disclosed sequences is preferably a sequence complement thereto according to the base pairing of nucleic acid molecules well known to the skilled person. For example, the bases A and G undergo base pairing with the bases T and U or C, resp. and visa versa. Modifications of the bases can influence the base-pairing partner.

The nucleic acid molecule of the invention comprises a nucleotide sequence which is at least about 30%, 35%, 40%, 45%, 50%, 55%, 60%, 65%, 70%, 75%, 80%, 90%, 95%, 96%, 97%, 98%, 99%, 99.5% or more homologous to a nucleotide sequence shown in Table I, application no. 16, columns 5 or 8, preferably the coding region thereof, homologs or fragments thereof, and preferably has above-mentioned activity, in particular conferring the production or the increased production of the fine chemical coenzyme Q10, coenzyme Q9, lipoic acid, or nicotinic acid, respectively, after increasing the activity or an activity of a gene as shown in the respective line in Table I or of a gene product, e.g. as shown in the respective line in Table II, application no. 16, column 5 or 8, by for example in one embodiment expression either in the cytosol or in an organelle such as a plastid or mitochondria or both, preferably in a plastid, or in another embodiment non-targeted or targeted.

The nucleic acid molecule of the invention comprises a nucleotide sequence or molecule which hybridizes, preferably hybridizes under stringent conditions as defined herein, to one of the nucleic acid molecule shown in the respective line in Table I, preferably Table IB, application no. 16, columns 5 or 8, preferably the coding region thereof, fragments or homologs thereof, and encodes a protein having above-mentioned activity, e.g. conferring the production or the increased production of the respective fine chemical coenzyme Q10, coenzyme Q9, lipoic acid, or nicotinic acid as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted, and optionally, the activity thereof is selected from the group consisting of 2-C-methyl-D-erythritol 4-phosphate cytidylyltransferase, acetolactate synthase III large subunit, acetyltransferase, acid shock protein, amino acid ABC transporter permease protein, amino acid acetyltransferase, At1g47380-protein, ATP synthase subunit E, ATP-binding component of a transport system, auxin response factor, AX653549-protein, AY087308-protein, b1003-protein, b1097-protein, b1522-protein, b2032-protein, b2739-protein, b2849-protein, b3817-protein, b4029-protein, beta-galactosidase, calcium-dependent protein kinase, cation-transporting ATPase, CCAAT-binding transcription factor, cell division control protein, colanic acid biosynthesis protein, CTP synthase, cyclin D , disease resistance protein, enoyl-CoA hydratase, fatty acid desaturase, F-box protein, flavodoxin, gluconate transport system permease 3, glutamine amidotransferase, glutaredoxin, glycerol dehydrogenase, glycerol-3-phosphate dehydrogenase, glycosyl transferase, halotolerance protein, heat shock protein Hsp40, heat shock transcription factor, histone acetyltransferase, hydrolase, isopropylmalate isomerase large subunit, L-aspartate oxidase, lipase, MADS box protein transcription factor, malate dehydrogenase, methylglyoxal synthase, methyltransferase, mitogen-activated protein kinase, monothiol glutaredoxin, multiple antibiotic resistance protein, N-acetyl-gamma-glutamylphosphate reductase, NADH dehydrogenase I chain I, oxidoreductase, permease protein of ABC transporter, phenylalanine ammonia-lyase, phosphoadenosine phosphosulfate reductase, phosphopantothenoylcysteine decarboxylase, photosystem II protein, protein kinase, protein phosphatase, pyruvate kinase, recombinase A, Ribose import ATP-binding protein, Sec-independent protein translocase subunit, serine protease, serine/threonine-protein phosphatase, sll1761-protein, slr1107-protein, sterol desaturase, threonine synthase, transcription factor, transcriptional regulator, TTC1386-protein, uroporphyrin-III C-methyltransferase, ycl027w-protein, zinc finger protein, and zinc transporter, respectively.

Moreover, the nucleic acid molecule of the invention can comprise only a portion of the coding region of one of the nucleic acid molecules depicted in Table I, application no. 16, columns 5 or 8, for example a fragment which can be used as a probe or primer or a fragment encoding a biologically active portion of the polypeptide of the present invention or of a polypeptide used in the process of the present invention, i.e. having above-mentioned activity, e.g. conferring the production or the increased production of the respective fine chemical coenzyme Q10, coenzyme Q9, lipoic acid, or nicotinic acid as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted. The nucleotide sequences determined from the cloning of the present protein-according-to-the-invention-encoding gene allows for the generation of probes and primers designed for use in identifying and/or cloning its homologues from other cell types and organisms. The probe/primer typically comprises a substantially purified oligonucleotide. The oligonucleotide typically comprises a region of nucleotide sequence that hybridizes under stringent conditions to at least about 12, 15 preferably about 20 or 25, more preferably about 40, 50 or 75 consecutive nucleotides of a sense strand of one of the sequences set forth, e.g., in the respective line in Table I, application no. 16, column 5 or 8, preferably the coding region thereof, or a homolog or a fragment thereof, an anti-sense sequence of one of the sequences, e.g., set forth in the respective line in Table I, application no. 16, columns 5 or 8, preferably the coding region thereof, or a homolog or a fragment thereof or naturally occurring mutants thereof. Primers based on a nucleic acid molecule of invention can be used in PCR reactions to clone homologs of the polypeptide of the invention or of the polypeptide used in the process of the invention, e.g. as the primers described in the examples of the present invention, e.g. as shown in the examples. A PCR with the primers shown in the respective line in Table III, column 8 will result in a fragment of the gene product as shown in Table II, application no. 16, column 5.

Primer sets are interchangeable. The person skilled in the art knows how to design and combine said primers to result in the desired product, e.g. in a full length clone or a partial sequence. Probes based on the sequences of the nucleic acid molecule of the invention or used in the process of the present invention can be used to detect transcripts or genomic sequences encoding the same or homologous proteins. The probe can further comprise a label group attached thereto, e.g. the label group can be a radioisotope, a fluorescent compound, an enzyme, or an enzyme co-factor. Such probes can be used as a part of a genomic marker test kit for identifying cells which express a polypeptide of the invention or used in the process of the present invention, such as by measuring a level of an encoding nucleic acid molecule in a sample of cells, e.g. detecting mRNA levels or determining, whether a genomic gene comprising the sequence of the polynucleotide of the invention or used in the processes of the present invention has been mutated or deleted.

As the expression of a respective nucleic acids of the invention is related to the synthesis of the respective fine chemical coenzyme Q10, coenzyme Q9, lipoic acid, or nicotinic acid its function as a probe extends to the detection of microorganisms, plant tissues, plants, plant variets, plant ecotypes or plant genera with varying capability or potential for synthesis of the respective fine chemical coenzyme Q10, coenzyme Q9, lipoic acid, or nicotinic acid. Therefore in one embodiment the present invention relates to a method for analyzing the capability or potential of a plant tissue, a plant, a plant variety or plant ecotype to produce the fine chemical coenzyme Q10, coenzyme Q9, lipoic acid, or nicotinic acid by using the nucleic acid of the invention or parts thereof as a probe to detect the amount of the nucleic acid of the invention in the non-human organism or a part thereof in comparison to another non-human organism.

The nucleic acid molecule of the invention encodes a polypeptide or portion thereof which includes an amino acid sequence which is sufficiently homologous to the amino acid sequence shown in respective line in Table II, application no. 16, columns 5 or 8 such that the protein or portion thereof maintains the ability to confer the production or the increased production of the respective fine chemical coenzyme Q10, coenzyme Q9, lipoic acid, or nicotinic acid as compared to a corresponding, e.g. non-transformed, wild type non-human organism, like a microorganism or a plant cell, plant or part thereof, in particular increasing the activity as mentioned above or as described in the examples in microorganisms or plants.

As used herein, the language “sufficiently homologous” refers to proteins or portions thereof which have amino acid sequences which include a minimum number of identical or equivalent amino acids (e.g. an amino acid which has a similar side chain as an amino acid in one of the sequences of the polypeptide of the present invention) to an amino acid sequence shown in the respective line in Table II, application no. 16, columns 5 or 8 such that the protein or portion thereof is able to confer the production or the increased production of the respective fine chemical coenzyme Q10, coenzyme Q9, lipoic acid, or nicotinic acid as compared to a corresponding, e.g. non-transformed, wild type non-human organism, like a microorganism or a plant cell, plant or part thereof. For example having the activity of a protein as shown in the respective line in Table II, application no. 16, column 3 and as described herein.

In an embodiment of the present invention, the nucleic acid molecule of the present invention comprises a nucleic acid molecule that encodes a portion of the protein of the present invention. The protein is at least about 30%, 35%, 40%, 45% 50%, 55%, 60%, 65%, 70%, 75%, 80%, 85%, 90%, 91%, 92%, 93%, 94%, 95%, 96%, 97%, 98%, 99%, 99.5% or more homologous to an entire amino acid sequence shown in the respective line of Table II, application no. 16, column 5 or 8 and has the above-mentioned activity, e.g. conferring the production or the increased production of the respective fine chemical coenzyme Q10, coenzyme Q9, lipoic acid, or nicotinic acid as compared to a corresponding, e.g. non-transformed, wild type non-human organism, like a microorganism or a plant cell, plant or part thereof by, for example in a embodiment expression either in the cytosol or in an organelle such as a plastid or mitochondria or both, preferably in a plastid, or in another embodiment by targeted or non-targeted expression

for the disclosure of this paragraph see [0338.1.7.7] above

for the disclosure of this paragraph see [0339.1.7.7] above

The invention further relates to nucleic acid molecules or methods using said nucleic acid molecules that differ from one of the nucleotide sequences shown in the respective line in Table I, application no. 16, columns 5 or 8, preferably the coding region thereof, or fragments or homologs thereof, due to degeneracy of the genetic code but encode a polypeptide of the present invention, in particular a polypeptide having the above-mentioned activity, e.g. as those polypeptides depicted in the respective line in Table II, application no. 16, columns 5 or 8, fragments or homologs thereof. Advantageously, the nucleic acid molecule of the invention comprises, or in an other embodiment has, a nucleotide sequence encoding a protein comprising, or in an other embodiment having, an amino acid sequence shown in the respective line in Table II, application no. 16, columns 5 or 8, fragments or homologs thereof. In a further embodiment, the nucleic acid molecule of the invention encodes a full length protein which is substantially homologous to an amino acid sequence shown in the respective line in Table II, application no. 16, columns 5 or 8, or homologs thereof. However, in an embodiment, the nucleic acid molecule of the present invention does not consist of the sequence shown in the respective line in Table I, preferably Table IA, application no. 16, columns 5 or 8, preferably the coding region thereof, but the functional homologs thereof.

[0341.1.7.16] to [0343.1.7.16] for the disclosure of these paragraphs see [0341.1.7.7] to [0343.1.7.7] above

Accordingly, in another embodiment, a nucleic acid molecule of the invention is at least 15, 20, 25 or 30 nucleotides in length. Preferably, it hybridizes under stringent conditions to a nucleic acid molecule comprising a nucleotide sequence of the nucleic acid molecule of the present invention or used in the process of the present invention, e.g. comprising the sequence shown in the respective line in Table I, application no. 16, columns 5 or 8, preferably the coding region thereof. In another embodiment the nucleic acid molecule is preferably at least 20, 30, 50, 100, 250 or more nucleotides in length.

for the disclosure of this paragraph see [0345.1.7.7] above

Preferably, a nucleic acid molecule of the invention that hybridizes under stringent conditions to a sequence shown in the respective line in Table I, application no. 16, columns 5 or 8, preferably the coding region thereof, corresponds to a naturally-occurring nucleic acid molecule of the invention. As used herein, a “naturally-occurring” nucleic acid molecule refers to a RNA or DNA molecule having a nucleotide sequence that occurs in nature (e.g.

encodes a natural protein). Preferably, the nucleic acid molecule encodes a natural protein having above-mentioned activity, e.g. conferring the production or the increased production of the respective fine chemical coenzyme Q10, coenzyme Q9, lipoic acid, or nicotinic acid as compared to a corresponding, e.g. non-transformed, wild type non-human organism, like a microorganism or a plant cell, plant or part thereof after increasing the expression or activity thereof or the activity of a protein of the invention or used in the process of the invention, in an embodiment for example expression either in the cytosol or in an organelle such as a plastid or mitochondria, preferably in plastids, or, in another embodiment by targeted or non-targeted expression.

for the disclosure of this paragraph see [0347.1.7.7] above

For example, nucleotide substitutions leading to amino acid substitutions at “non-essential” amino acid residues can be made in a sequence of the nucleic acid molecule of the invention or used in the process of the invention, e.g. shown in the respective line in Table I, application no. 16, columns 5 or 8, preferably the coding region thereof, or fragments or homologs thereof.

for the disclosure of this paragraph see [0349.1.7.7] above

for the disclosure of this paragraph see [0350.1.7.7] above

Accordingly, the invention relates to nucleic acid molecules encoding a polypeptide having above-mentioned activity, in a non-human organisms or parts thereof by for example expression either in the cytosol or in an organelle such as a plastid or mitochondria or both, preferably in plastids, that contain changes in amino acid residues that are not essential for said activity. Such polypeptides differ in amino acid sequence from a sequence depicted in the respective line in Table II, especially Table IIA, application no. 16, columns 5 or 8, but retain said activity described herein. The nucleic acid molecule can comprise a nucleotide sequence encoding a polypeptide, wherein the polypeptide comprises an amino acid sequence at least about 50% identical to an amino acid sequence shown in the respective line in Table II, application no. 16, columns 5 or 8, and is capable of conferring the production or the increased production of the respective fine chemical coenzyme Q10, coenzyme Q9, lipoic acid, or nicotinic acid as compared to a corresponding, e.g. non-transformed, wild type non-human organism, like a microorganism or a plant cell, plant or part thereof, after increasing its activity for example in an embodiment by expression either in the cytosol or in an organelle such as a plastid or mitochondria or both, preferably in plastids, or, in another embodiment by targeted or non-targeted expression. Preferably, the protein encoded by the nucleic acid molecule is at least about 60%, 70%, 80%, 90%, 95%, 96%, 97%, 98%, 99% or 99.5% identical to the sequence shown in the respective line in Table II, application no. 16, columns 5 or 8.

[0352.1.7.16] to [0357.1.7.16] for the disclosure of these paragraphs see [0352.1.7.7] to [0357.1.7.7] above

Functional equivalents of the nucleic acid molecules according to the present invention and/or used in the process according to the present invention represent an embodiment of homologs. Functional equivalents derived from the nucleic acid molecules as shown in the respective line in Table I, application no. 16, columns 5 or 8, preferably the coding region thereof, according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the nucleic acid molecules as shown in the respective line in Table I, application no. 16, columns 5 or 8, preferably the coding region thereof, according to the invention and encode polypeptides having essentially the same properties as the polypeptide as shown in the respective line in Table II, application no. 16, columns 5 or 8.

Functional equivalents derived from one of the polypeptides as shown in the respective line in Table II, application no. 16, columns 5 or 8 according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the polypeptides as shown in the respective line in Table II, application no. 16, columns 5 or 8 according to the invention and having essentially the same properties as the polypeptide as shown in the respective line in Table II, application no. 16, columns 5 or 8.

for the disclosure of this paragraph see [0359.1.7.7] above

A nucleic acid molecule encoding a homolog to a polypeptide depicted in the respective line in Table II, application no. 16, columns 5 or 8 can be created by introducing one or more nucleotide substitutions, additions or deletions into the nucleic acid molecule of the present invention, in particular of Table I, application no. 16, columns 5 or 8, in the respective line, such that one or more amino acid substitutions, additions or deletions are introduced into the encoded protein. Mutations can be introduced into the nucleic acid molecules as depicted in the respective line in Table I, application no. 16, columns 5 or 8, preferably the coding region thereof, by standard techniques, such as site-directed mutagenesis, PCR-mediated mutagenesis or other methods known to the person skilled in the art.

[0361.1.7.16] to [0363.1.7.16] for the disclosure of these paragraph see [0363.1.7.7] above

for the disclosure of this paragraph see [0361.1.7.7] above

Homologues of the nucleic acid sequences used, with the sequences shown in the respective line in Table I, application no. 16, columns 5 or 8, preferably the coding region thereof, comprise also allelic variants with at least approximately 30%, 35%, 40%, 45%, 50%, 55%, 60%, 65%, 70%, 75%, 80%, 85%, 90%, 91%, 92%, 93%, 94% 95%. 96%, 97%, 98%, 99%, 99.5% or more % homology with one of the nucleotide sequences shown or the above-mentioned derived nucleic acid sequences or their homologues or derivatives or parts of these. Allelic variants encompass in particular functional variants which can be obtained by deletion, insertion or substitution of nucleotides from the sequences shown in the respective line, preferably in Table I, columns 5 or 8, preferably the coding region thereof, or from the derived nucleic acid sequences, the intention being, however, that the enzymatic activity or the biological activity of the resulting proteins synthesized is advantageously retained or increased.

In one embodiment of the present invention, the nucleic acid molecule of the invention or used in the process of the invention comprises the sequences shown in the respective line in any of the Table I, application no. 16, columns 5 or 8, preferably the coding region thereof, fragments or homologs thereof. It is preferred that the nucleic acid molecule comprises as little as possible other nucleotides not shown in the respective line in any one of Table I, application no. 16, columns 5 or 8, preferably the coding region thereof, fragments or homologs thereof. In one embodiment, the nucleic acid molecule comprises less than 500, 400, 300, 200, 100, 90, 80, 70, 60, 50 or 40 further nucleotides. In a further embodiment, the nucleic acid molecule comprises less than 30, 20 or 10 further nucleotides. In one embodiment, the nucleic acid molecule used in the process of the invention is identical to the sequences shown in the respective line in Table I, application no. 16, columns 5 or 8, preferably the coding region thereof.

Also preferred is that the nucleic acid molecule used in the process of the invention encodes a polypeptide comprising the sequence shown in the respective line in Table II, application no. 16, columns 5 or 8, fragments or homologs thereof. In one embodiment, the nucleic acid molecule encodes less than 150, 130, 100, 80, 60, 50, 40 or 30 further amino acids. In a further embodiment, the encoded polypeptide comprises less than 20, 15, 10, 9, 8, 7, 6 or 5 further amino acids. In one embodiment used in the inventive process, the encoded polypeptide is identical to the sequences shown in the respective line in Table II, application no. 16, columns 5 or 8.

In one embodiment, the nucleic acid molecule of the invention or used in the process encodes a polypeptide comprising the sequence shown in the respective line in Table II, application no. 16, columns 5 or 8, or fragments or homologs thereof, and comprises less than 100 further nucleotides. In a further embodiment, said nucleic acid molecule comprises less than 30 further nucleotides. In one embodiment, the nucleic acid molecule used in the process is identical to a coding sequence of the sequences shown in the respective line in Table I, application no. 16, columns 5 or 8.

Polypeptides (=proteins), which still have the essential biological or enzymatic activity of the polypeptide of the present invention conferring the production or the increased production of the respective fine chemical coenzyme Q10, coenzyme Q9, lipoic acid, or nicotinic acid as compared to a corresponding, e.g. non-transformed, wild type non-human organism, like a microorganism or a plant cell, plant or part thereof, i.e. whose activity is essentially not reduced, are polypeptides with at least 10%, 20%, 30%, 40%, 50%, 60%, 70%, 80%, 90%, 95% or more of the wild type biological activity or enzymatic activity, advantageously, the activity is essentially not reduced in comparison with the activity of a polypeptide shown in the respective line in Table II, application no. 16, columns 5 or 8 expressed under identical conditions.

Homologues of nucleic acid molecules shown in the respective line in Table I, application no. 16, columns 5 or 8, preferably the coding region thereof, or fragments thereof, or of the derived sequences shown in the respective line in Table II, application no. 16, columns 5 or 8, or fragments thereof, also mean truncated sequences, cDNA, single-stranded DNA or RNA of the coding and noncoding DNA sequence. Homologues of said sequences are also understood as meaning derivatives, which comprise noncoding regions such as, for example, UTRs, introns, terminators, enhancers or promoter variants. The promoters upstream of the nucleotide sequences stated can be modified by one or more nucleotide substitution(s), insertion(s) and/or deletion(s) without, however, interfering with the functionality or activity either of the promoters, the open reading frame (=ORF) or with the 3′-regulatory region such as terminators or other 3′-regulatory regions, which are far away from the ORF. It is furthermore possible that the activity of the promoters is increased by modification of their sequence, or that they are replaced completely by more active promoters, even promoters from heterologous organisms.

Appropriate promoters are known to the person skilled in the art and are mentioned herein below.

[0370.1.7.16] to [0379.1.7.16] for the disclosure of these paragraphs see [0370.1.7.7] to [0379.1.7.7] above

Moreover, a native polypeptide conferring the increase of the respective fine chemical coenzyme Q10, coenzyme Q9, lipoic acid, or nicotinic acid in a non-human organism or a part thereof can be isolated from cells (e.g., endothelial cells), for example using the antibody of the present invention as described below, in particular, an antibody against proteins having 2-C-methyl-D-erythritol 4-phosphate cytidylyltransferase, acetolactate synthase III large subunit, acetyltransferase, acid shock protein, amino acid ABC transporter permease protein, amino acid acetyltransferase, At1g47380-protein, ATP synthase subunit E, ATP-binding component of a transport system, auxin response factor, AX653549-protein, AY087308-protein, b1003-protein, b1097-protein, b1522-protein, b2032-protein, b2739-protein, b2849-protein, b3817-protein, b4029-protein, beta-galactosidase, calcium-dependent protein kinase, cation-transporting ATPase, CCAAT-binding transcription factor, cell division control protein, colanic acid biosynthesis protein, CTP synthase, cyclin D , disease resistance protein, enoyl-CoA hydratase, fatty acid desaturase, F-box protein, flavodoxin, gluconate transport system permease 3, glutamine amidotransferase, glutaredoxin, glycerol dehydrogenase, glycerol-3-phosphate dehydrogenase, glycosyl transferase, halotolerance protein, heat shock protein Hsp40, heat shock transcription factor, histone acetyltransferase, hydrolase, isopropylmalate isomerase large subunit, L-aspartate oxidase, lipase, MADS box protein transcription factor, malate dehydrogenase, methylglyoxal synthase, methyltransferase, mitogen-activated protein kinase, monothiol glutaredoxin, multiple antibiotic resistance protein, N-acetyl-gamma-glutamylphosphate reductase, NADH dehydrogenase I chain I, oxidoreductase, permease protein of ABC transporter, phenylalanine ammonia-lyase, phosphoadenosine phosphosulfate reductase, phosphopantothenoylcysteine decarboxylase, photosystem II protein, protein kinase, protein phosphatase, pyruvate kinase, recombinase A, Ribose import ATP-binding protein, Sec-independent protein translocase subunit, serine protease, serine/threonine-protein phosphatase, sll1761-protein, slr1107-protein, sterol desaturase, threonine synthase, transcription factor, transcriptional regulator, TTC1386-protein, uroporphyrin-III C-methyltransferase, ycl027w-protein, zinc finger protein, or zinc transporter activity, respectively, or an antibody against polypeptides as shown in the respective line in Table II, application no. 16, columns 5 or 8, or fragments or homologs thereof which can be produced by standard techniques utilizing the polypeptid of the present invention or fragment thereof, i.e., the polypeptide of this invention (FCRP). Preferred are monoclonal antibodies.

for the disclosure of this paragraph see [0381.1.7.7] above

In one embodiment, the present invention relates to a polypeptide as depicted in the respective line in Table II, especially in Table IIA or especially in Table IIB, application no. 16, columns 5 or 8, or fragment or homolog thereof or as coded by the nucleic acid molecule shown in the respective line in Table I, especially Table IA or especially Table IB, application no. 16, columns 5 or 8, preferably the coding region thereof, or fragment or homolog thereof.

In an embodiment, the present invention relates to a polypeptide comprising or consisting of a consensus sequence shown in the respective line in Table IV, application no. 16, column 8 or at least one polypeptide motif shown in the respective line in Table IV, application no. 16, column 8.

In an embodiment thereof, the present invention relates to a polypeptide comprising or consisting of a consensus sequence shown in the respective line in Table IV, application no. 16, column 8 or comprising at least one polypeptide motif shown in the respective line in Table IV, application no. 16, column 8, whereby not more than 20, 15, 10, 9, 8, 7, 6, 5, 4, 3, 2, or 1, or 0 of the amino acids can be replaced by any amino acid.

In another embodiment thereof, the present invention relates to a polypeptide comprising or consisting of a consensus sequence shown in the respective line in Table IV, application no. 16, column 8 or comprising at least one polypeptide motif shown in the respective line in Table IV, application no. 16, column 8, whereby not more than 15%, 10%, 5%, 4%, 3%, 2% or1% or 0% of the amino acids defined as distinct specific amino acids are/is replaced by another amino acid.

In another embodiment thereof, the present invention relates to a polypeptide comprising or consisting of a consensus sequence shown in the respective line in Table IV, application no. 16, column 8 or comprising at least one polypeptide motif shown in the respective line in Table IV, application no. 16, column 8, whereby not more than 20, 15, 10, 9, 8, 7, 6, 5, 4, 3, 2, or 1, or 0 amino acids are inserted into the consensus sequence or polypeptide motif.

In an embodiment of the present invention, the polypeptide of the present invention comprises or consists of a consensus sequence or at least one polypeptide motif shown in the respective line in Table IV, application no. 16, column 8, whereby said polypeptide distinguishes over the sequence depicted in the respective line in Table II, application no. 16, columns 5 or 8 by one or more amino acids. In one embodiment, said polypeptide of the invention distinguishes over the sequence shown in the respective line in Table II, application no. 16, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 16, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in the respective line in Table II, application no. 16, columns 5 and 8.

In an embodiment, the invention relates to a polypeptide conferring an increase in the respective fine chemical coenzyme Q10, coenzyme Q9, lipoic acid, or nicotinic acid in a non-human organism, especially a microorganism or a plant, or a part thereof, being encoded by the nucleic acid molecule of the invention or used in the process of the invention and having a sequence which distinguishes over the sequence as shown in the respective line in Table II, application no. 16, columns 5 or 8 by one or more amino acids (but not exceeding 5%, preferably 4%, 3%, 2%, 1%, 0.5%, 0.2% or 0.1%). In an embodiment, said polypeptide of the invention does not comprise or consist of the sequence shown in the respective line in Table II, application no. 16, columns 5 or 8. In an embodiment, said polypeptide of the present invention is less than 100%, 99.999%, 99.99%, 99.9% or 99% identical. In one embodiment, said polypeptide which differs at least in one or more amino acids (but not exceeding 5%, preferably 4%, 3%, 2%, 1%, 0.5%, 0.2% or 0.1%) from the polypeptide shown in the respective line in Table II, application no. 16, columns 5 and 8 does not comprise a protein of the sequence shown in the respective line in Table II A and/or II B, application no. 16, columns 5 or 8.

In an embodiment, the present invention relates to a polypeptide having the activity of the protein as shown in the respective line in Table II, application no. 16, column 3, which distinguishes over the sequence depicted in the respective line in Table II, application no. 16, columns 5 or 8 by one or more amino acids, preferably by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 16, columns 5 and 8 by not more than 80%, 70%, 60%, 50%, 40%, 30%, 20%, 10%, 5%, 4%, 3%, 2%, 1% or 0.5%.

In another embodiment the polypeptide of the invention takes the form of a preprotein consisting of a plastidial or mitochondrial transit peptide joint to a polypeptide having the activity of the protein as shown in Table II, column 3, from which the transit peptide is preferably cleaved off upon transport of the preprotein into the organelle for example into the plastid or mitochondrion.

In another embodiment the polypeptide of the invention takes not the form of a preprotein.

for the disclosure of this paragraph see [0390.1.7.7] above

for the disclosure of this paragraph see [0391.1.7.7] above

A polypeptide of the invention can participate in the process of the present invention. The polypeptide or a portion thereof comprises preferably an amino acid sequence, which is sufficiently homologous to an amino acid sequence shown in the respective line in Table II A and/or II B, application no. 16, columns 5 or 8.

Further, the polypeptide can have an amino acid sequence which is encoded by a nucleotide sequence which hybridizes, preferably hybridizes under stringent conditions as described above, to a nucleotide sequence of the nucleic acid molecule of the present invention. Accordingly, the polypeptide has an amino acid sequence which is encoded by a nucleotide sequence that is at least about 35%, 40%, 45%, 50%, 55%, 60%, 65%, 70%, 75%, 80%, 85%, 90%, 91%, 92%, 93%, 94%, 95%, 96%, 97%, 98%, 99%, 99.5% or more homologous to one of the amino acid sequences as shown in the respective line in Table II A and/or II B, application no. 16, columns 5 or 8, or fragments thereof. The preferred polypeptide of the present invention preferably possesses at least one of the activities according to the invention and described herein. A preferred polypeptide of the present invention includes an amino acid sequence encoded by a nucleotide sequence which hybridizes, preferably hybridizes under stringent conditions, to a nucleotide sequence shown in the respective line in Table I A and/or I B, application no. 16, columns 5 or 8, preferably the coding region thereof, or fragments thereto or which is homologous thereto, as defined above

The polypeptide of the present invention can vary from the sequences shown in the respective line in Table II A and/or II B, application no. 16, columns 5 or 8 in amino acid sequence due to natural variation or mutagenesis, as described in detail herein. Accordingly, the polypeptide comprise an amino acid sequence which is at least about 35%, 40%, 45%, 50%, 55%, 60%, 65%, 70%, 75%, 80%, 85% or 90, 91%, 92%, 93%, 94%, 95%, 96%, 97%, 98%, 99%, 99.5% or more homologous to an entire amino acid sequence shown in the respective line in Table II A and/or II B, application no. 16, columns 5 or 8.

Biologically active portions of a polypeptide of the present invention include peptides comprising amino acid sequences derived from the amino acid sequence of the polypeptide of the present invention or used in the process of the present invention, e.g., the amino acid sequence shown in the respective line in Table II, application no. 16, columns 5 or 8 or the amino acid sequence of a protein homologous thereto, which include fewer amino acids than a full length polypeptide of the present invention or used in the process of the present invention or the full length protein which is homologous to a polypeptide of the present invention or used in the process of the present invention depicted herein, and exhibit at least one activity of a polypeptide of the present invention or used in the process of the present invention.

for the disclosure of this paragraph see [0396.1.7.7] above

Manipulation of the nucleic acid molecule of the invention may result in the production of a protein having differences from the protein as shown in the respective line in Table II, application no. 16, column 5 or 8. Differences shall mean at least one amino acid different from the sequences as shown in the respective line in Table II, application no. 16, column 5 or 8, preferably at least 2, 3, 4, 5, 6, 7, 8, 9 or 10 amino acids (especially but not exceeding 0.1%, preferably 0.2%, 0.5%, 1%, 2%, 3% 4% or 5%), more preferably at least 15, 20, 25, 30, 35, 40, 45 or 50 amino acids (especially but not exceeding 0.5%, preferably 1%, 2%, 3% 4%, 5%, 10% or 25%) different from the sequences as shown in the respective line in Table II, application no. 16, column 5 or 8. These proteins may be improved in efficiency or activity, or may be more stable and therefore present in greater numbers in the cell as compared to the wild-type cell.

Any mutagenesis strategies for the polypeptide of the present invention or the polypeptide used in the process of the present invention to result in increasing said activity are not meant to be limiting; variations on these strategies will be readily apparent to one skilled in the art. Using such strategies, and incorporating the mechanisms disclosed herein, the nucleic acid molecule and polypeptide of the invention may be utilized to generate algae, ciliates, fungi, other microorganisms like C. glutamicum or plants or parts thereof, expressing proteins as shown in the respective line in Table II, application no. 16, column 5 or 8, or mutated proteins thereof. The nucleic acid molecules and polypeptide molecules of the invention are expressed such that the yield, production, and/or efficiency of production of a desired compound is improved.

[0399.1.7.16] to [0401.1.7.16] for the disclosure of these paragraphs see [0399.1.7.7] to [0401.1.7.7] above

In one embodiment, a protein (=polypeptide) as shown in the respective line in Table II, application no. 16, column 5 or 8 refers to a polypeptide having an amino acid sequence corresponding to the polypeptide of the invention, especially to a polypeptide as shown in Table II, application no. 16, columns 5 or 8 or homologs thereof, or used in the process of the invention, whereas a “non- inventive protein or polypeptide” or “other polypeptide” refers to a polypeptide having an amino acid sequence corresponding to a protein which is not substantially homologous to a polypeptide of the invention, preferably which is not substantially homologous to a polypeptide or protein as shown in the respective line in Table II, application no. 16, column 5 or 8 or homologs thereof, e.g., a protein which does not confer the activity described herein and which is derived from the same or a different organism.

[0403.1.7.16] to [0409.1.7.16] for the disclosure of theses paragraphs see [0403.1.7.7] to [0409.1.7.7] above

As the nucleic acid molecules of the invention and/or the expression thereof is related to the synthesis of the respective fine chemical coenzyme Q10, coenzyme Q9, lipoic acid, or nicotinic acid its function as a probe extends to the detection of microorganisms, plant tissues, plants, plant variets, plant ecotypes or plant genera with varying, advantageously increased, capability or potential for synthesis of the respective fine chemical coenzyme Q10, coenzyme Q9, lipoic acid, or nicotinic acid. Therefore in one embodiment the present invention relates to a method for analyzing the capability or potential of a plant tissue, a plant, a plant variety or ecotype to produce the respective fine chemical coenzyme Q10, coenzyme Q9, lipoic acid, or nicotinic acid by using the respective antibody of the invention as a probe to detect the amount of the polypeptide encoded by said nucleic acid molecule of the invention in a non-human organism in comparison to another organism.

Therefore in one embodiment the invention relates to a method to probe a non-human organism for its capability for the production of the respective fine chemical comprising the steps of
a) analyzing the sequence and/or the expression of the respective nucleic acid molecule of the invention in least two non-human organisms which differ in the capability to produce the respective fine chemical;
b) associate the different sequence and/or expression level of the nucleic acid molecules of the invention with their different capability to produce the respective fine chemical;
c) analyze the sequence and/or the expression of the nucleic acid molecule of the invention in at least a third non-human organism;
d) predict the capability of the at least third organism to produce the respective fine chemical based by comparing the sequence and/or expression of the nucleic acid of the invention to the sequences and/or expression levels of the nucleic acid molecule of the invention of the at least two organisms analyzed in step a.

[0411.1.7.16] to [0430.1.7.16] for the disclosure of these paragraphs see [0411.1.7.7] to [0430.1.7.7] above

Accordingly the present invention relates to any cell transgenic for any nucleic acid characterized as part of the invention, e.g. conferring the increase of the respective fine chemical coenzyme Q10, coenzyme Q9, lipoic acid, or nicotinic acid in a cell or a non-human organism or a part thereof, e.g. the nucleic acid molecule of the invention, the nucleic acid construct of the invention, the vector of the invention, the expression cassette according to the invention, or a nucleic acid molecule encoding the polypeptide of the invention, e.g. encoding a polypeptide having an activity as the protein as shown in the respective line in Table II, application no. 16, column 3. Due to the above-mentioned activity the respective fine chemical coenzyme Q10, coenzyme Q9, lipoic acid, or nicotinic acid content in a cell or a non-human organism is increased. For example, due to modulation or manipulation, the cellular activity is increased, in a preferred embodiment in organelles such as plastids or mitochondria, e.g. due to an increased expression or specific activity or specific targeting of the subject matters of the invention in a cell or a non-human organism or a part thereof especially in organelles such as plastids or mitochondria, or in another embodiment in the cytosol. Transgenic for a polypeptide having a protein or a protein activity means herein that due to modulation or manipulation of the genome, the activity of protein as shown in the respective line in Table II, application no. 16, column 3 or a protein as shown in the respective line in Table II, application no. 16, column 3-like activity is increased in the cell or non-human organism or part thereof, especially in organelles such as plastids or mitochondria, or especially in the cytosol. Examples are described above in context with the process of the invention.

for the disclosure of this paragraph see [0432.1.7.7] above

A naturally occurring expression cassette—for example the naturally occurring combination of the promoter of the gene encoding a protein as shown in the respective line in Table II, application no. 16, column 5 or 8, preferably the coding region thereof, or fragments or homologs thereof with the corresponding encoding gene—becomes a transgenic expression cassette when it is modified by non-natural, synthetic “artificial” methods such as, for example, mutagenesis. Such methods have been described (U.S. Pat. No. 5,565,350; WO 00/15815; also see above).

for the disclosure of this paragraph see [0434.1.7.7] above

for the disclosure of this paragraph see [0435.1.7.7] above

Transgenic plants comprising the respective fine chemical(s) synthesized in the process according to the invention can be marketed directly without isolation of the fine chemical synthesized. In the process according to the invention, plants are understood as meaning all plant parts, plant organs such as leaf, stalk, root, tubers or seeds or propagation material or harvested material or the intact plant. In this context, the seed encompasses all parts of the seed such as the seed coats, epidermal cells, seed cells, endosperm or embryonic tissue. The fine chemical produced in the process according to the invention may, however, also be isolated from the plant. In case of coenzyme Q10, coenzyme Q9, lipoic acid, or nicotinic acid this can be in free form or bound to proteins. Fine chemical(s) produced by this process can be harvested by harvesting the non-human organisms either from the culture in which they grow or from the field. For example, this can be done via squeezing, grinding and/or extraction, salt precipitation and/or ion-exchange chromatography of the plant parts, preferably the plant seeds, plant fruits, plant tubers and the like.

[0437.1.7.16] to [0448.1.16.16] for the disclosure of these paragraphs see [0437.1.7.7] to [0448.1.16.7] above

The fine chemical obtained in the process are suitable as starting material for the synthesis of further products of value.

For example, in case of vitamins they can be used in combination with each other or alone for the production of pharmaceuticals, foodstuffs, animal feeds or cosmetics.

The respective fine chemical can then be processed further directly into foodstuffs or animal feeds or for other applications.

Coenzyme Q10 is cardioprotective, cytoprotective, and neuroprotective. It is an anti-oxidant and an important metabolic cofactor for the production of energy. Coenzyme Q10 is an effective adjunct treatment for various conditions of the cardiovascular system. Its most well recognized and studied uses lie within the conditions of congestive heart failure, hypertension, and angina.

For individuals with congestive heart failure, Coenzyme Q10 has been shown to increase the quality of life.

Coenzyme Q10 may improves symptoms in patients with high blood pressure, mitral valve prolapse, and angina. It is also protective against the development of atherosclerosis, by inhibiting the oxidation of LDL and by increasing HDL levels.

Coenzyme Q10 may be an effective addition to any cancer treatment protocol. Numerous studies have highlighted its ability to decrease the cardiac toxicity of a popular anti-cancer drug, doxorubicin; also known as Adriamycin.

Coenzyme Q10 may be a useful adjunctive treatment for individuals with breast cancer.

Coenzyme Q10 can reduce oxidative stress via its anti-oxidant action. It is theorized that by reducing oxidative stress, CoQ10 can protect against cardiovascular and neurological disease; among the many other conditions that may be related to free radical damage and oxidative stress.

It further can be helpful for individuals with early onset Parkinson's disease. It may slow the progression of disease and deterioration of cognitive function. [9] In one particular controlled study, individuals taking Coenzyme Q10 had less disability over time than those not taking Coenzyme Q10. Supplemental CoQ10 was well tolerated with few side effects in very high doses. Topical Coenzyme Q10 can be an effective treatment alone, or in combination with non-surgical therapy for peridontitis. It has been shown to improve symptoms of the disease and reduce the progression. Topical CoQ10 creams are also becoming popular in many cosmetic formulations.

In pharmacological doses, niacin has been proven to reverse atherosclerosis by reducing total cholesterol, triglycerides, very-low-density lipoprotein (VLDL), and low-density lipoprotein (LDL), and increasing high-density lipoprotein (HDL). Niacin may have the ability to lower lipoprotein (a), which is beneficial at reducing thrombotic tendency. It can be used in reducing carotid intima-media thickness, a marker of atherosclerosis.

Lipoic acid can improve glucose utilization in individuals with type 2 Diabetis Mellitus and can improve endothelium-dependent vasodilation. Intravenous and oral lipoic acid are approved for the treatment of diabetic neuropathy in Germany.

Feeding high doses of lipoic acid to mice with experimental autoimmune encephalomyelitis (EAE), a model of multiple sclerosis (MS), has been found to slow disease progression. Lipoic acid alone or in combination with other antioxidants or L-carnitine has been found to improve measures of memory in animal models of age-associated cognitive decline, including rats, mice and dogs.

[0450.1.7.16] to [0452.1.7.16] for the disclosure of theses paragraphs see [0450.1.7.7] to [0452.1.7.7] above

Further in another embodiment, the present invention relates to the use of the nucleic acid molecule of the invention or used in the method of the invention alone or in combination with other genes of the respective fine chemical synthesis for example of the cofactor biosynthesis, the polypeptide of the invention or used in the method of the invention, the nucleic acid construct of the invention, the expression cassette of the invention, the vector of the invention, the plant or plant tissue or the host cell of the invention, for the production of plant resistant to a herbicide inhibiting eventually the production of the fine chemical.

for the disclosure of this paragraph see [0454.1.7.7] above

In one embodiment, the present invention relates to a method for the identification of a gene product conferring the production of or an increase in the fine chemical production in a non-human cell, comprising the following steps:
(a) contacting, e.g. hybridizing, a, some or all nucleic acid molecules of a sample, e.g. cells, tissues, plants or microorganisms or a nucleic acid library, which can contain a candidate gene encoding a gene product conferring the production of or an increase in the fine chemical after expression, with the nucleic acid molecule of the present invention as shown in the respective line in column 5 or 8 of Table I A or B of application no. 16, preferably the coding region thereof, or a fragment or homolog thereof;
(b) identifying the nucleic acid molecules, which hybridize under relaxed stringent conditions with the nucleic acid molecule of the present invention in particular to the nucleic acid molecule sequence shown in the respective line in Table I, application no. 16, columns 5 or 8, preferably the coding region thereof, of a fragment or a homolog thereof, preferably in Table I B, application no. 16, columns 5 or 8, preferably the coding region thereof, of a fragment or a homolog thereof, and, optionally, isolating the full length cDNA clone or complete genomic clone;
(c) introducing the candidate nucleic acid molecules or a fragment thereof in host cells, preferably in a plant cell or a microorganism, appropriate for producing the fine chemical;
(d) expressing the identified nucleic acid molecules in the host cells for which the production of or the increased production of the fine chemical is desired;
(e) assaying the fine chemical level in the non-human host cells; and
(f) identifying the nucleic acid molecule and/or its gene product which expression confers the production of or an increase in the fine chemical level in the non-human host cell after expression compared to the wild type.

[0456.1.7.16] for the disclosure of this paragraph see [0456.1.7.7] above [0457.1.16.16] In another embodiment, the present invention relates to a method for the identification of a gene product conferring the production of or an increase in the fine chemical production in a cell, comprising the following steps:
(a) identifying a nucleic acid molecule of a non-human organism, which is at least 20%, 25%, 30%, 35%, 40%, 50%, 60%, 70%, 80%, 90%, 95%, 96%, 97%, 98%, 99%, 99.5% or more homolog to the nucleic acid molecule encoding a polypeptide comprising the polypeptide molecule as shown in the respective line in column 5 or 8 of Table II, application no. 16, or comprising a consensus sequence or a polypeptide motif as shown in the respective line in column 8 of Table IV, application no. 16, or being encoded by a nucleic acid molecule comprising a polynucleotide as shown in the respective line in column 5 or 8 of Table I, application no. 16, preferably the coding region thereof, or a homologue thereof as described herein, for example via homology search in a data bank;
(b) introducing the candidate nucleic acid molecule in non-human host cells, preferably in a plant cell or a microorganism, appropriate for producing the fine chemical;
(c) expressing the identified nucleic acid molecule in the non-human host cells;
(d) assaying the fine chemical level in the non-human host cells; and
(e) identifying the nucleic acid molecule and its gene product which expression confers the production of or an increase in the fine chemical level in the non-human host cell after expression compared to the wild type.

for the disclosure of this paragraph see [0458.1.7.7] above

Further, the nucleic acid molecules disclosed herein, in particular the nucleic acid molecules shown in the respective line in column 5 or 8 of Table I A or B of application no. 16, may be sufficiently homologous to the sequences of related species such that these nucleic acid molecules may serve as markers for the construction of a genomic map in related organism or for association mapping. Furthermore natural variation in the genomic regions corresponding to nucleic acids disclosed herein, in particular the nucleic acid molecule shown in the respective line in column 5 or 8 of Table I A or B, of application no. 16 or homologous thereof, may lead to variation in the activity of the proteins disclosed herein, in particular the proteins comprising polypeptides as shown in the respective lines in column 5 or 8 of Table II A or B, of application no. 16 or their homologous, or comprising the consensus sequence or the polypeptide motif as shown in the respective line in column 8 of Table IV, and in consequence in a natural variation of an increased fine chemical production.

In consequence natural variation eventually also exists in form of more active allelic variants leading already to a relative increase in the fine chemical. Different variants of the nucleic acid molecules disclosed herein, in particular the nucleic acid molecules comprising the nucleic acid molecules as shown in the respective line in column 5 or 8 of Table I A or B of application no. 16, preferably the coding region thereof, which corresponds to different levels of increase in fine chemical, e.g. different levels of increase in fine chemical, can be indentified and used for marker assisted breeding for an enhanced production of the fine chemical.

Natural variation may also exist in the regulatory regions, e.g. the promoters of the natural genes comprising the nucleic acid molecules as shown in the respective line column 5 or 8 of Table I A or B of application no. 16, preferably the coding region thereof, which can similarly correspond to different levels of increase in the fine chemical, e.g. different levels of increase in the fine chemical can be identified and used for marker assisted breeding for an enhanced production of the fine chemical.

Accordingly, the present invention relates to a method for breeding plants for the production of the fine chemical, comprising
(a) selecting a first plant variety with an increased production of the fine chemical, e.g. based on an increased expression of a nucleic acid molecule of the invention as disclosed herein, in particular of a nucleic acid molecule comprising a nucleic acid molecule as shown in the respective line in column 5 or 8 of Table I A or B of application no. 16, preferably the coding region thereof, or fragments or homologs thereof, or a polypeptide comprising a polypeptide as shown in the respective line in column 5 or 8 of Table II A or B of application no. 16, or fragments or homologs thereof, or comprising a consensus sequence or a polypeptide motif as shown in the respective line in column 8 of Table IV, or a homolog thereof, as described herein;
(b) associating the level of increased production of the fine chemical, with the expression level or the genomic structure of a gene encoding said polypeptide or said nucleic acid molecule;
(c) crossing the first plant variety with a second plant variety, which significantly differs in its level of fine chemical; and
(d) identifying, which of the offspring varieties has got increased production of the fine chemical by the expression level of said polypeptide or nucleic acid molecule or the genomic structure of the gene(s) encoding said polypeptide or nucleic acid molecule of the invention.

[0462.1.7.16] to [0479.1.7.16] for the disclosure of these paragraphs see [0462.1.7.7] to [0479.1.7.7] above

In a further embodiment the nucleic acid molecule of the invention, the polypeptide of the invention, the nucleic acid construct of the invention, the expression cassette of the invention, the non-human organisms, the non-human host cell, the microorganisms, the plant, plant cell, or the part thereof, of the invention, the vector of the invention, the agonist identified with the method of the invention, the nucleic acid molecule identified with the method of the present invention, can be used for the production of the fine chemical or of the fine chemical and one or more other cofactors.

Accordingly, the nucleic acid molecule of the invention, or the nucleic acid molecule identified with the method of the present invention or the complement sequences thereof, the polypeptide of the invention, the nucleic acid construct of the invention, the expression cassette of the invention, the non-human organism, the non-human host cell, the microorganism, the plant, plant cell, or the part thereof of the invention, the vector of the invention, the agonist identified with the method of the invention, the antibody of the present invention, can be used for the production of or the increase in the fine chemical in a non-human organism or part thereof, e.g. in a cell.

for the disclosure of this paragraph see [0481.1.7.7] above

for the disclosure of this paragraph see [0482.1.7.7] above

A special embodiment of this invention is related to genes which increase or generate the production of the fine chemical coenzyme Q10 and/or coenzyme Q9 in plant cells, plants or part thereof. Phenotypes thereto are associated with yield of plants (=yield related phenotypes). In accordance with the invention, therefore, the respective genes identified in Table I, wherein in column 7 coenzyme Q10 and/or coenzyme Q9 are mentioned, especially the coding region thereof, or homologs or fragments thereof,may be employed to enhance any yield-related phenotype. Increased yield may be determined in field trials of transgenic plants and suitable control plants. Alternatively, a transgene's ability to increase yield may be determined in a model plant. An increased yield phenotype may be determined in the field test or in a model plant by measuring any one or any combination of the following phenotypes, in comparison to a control plant: yield of dry harvestable parts of the plant, yield of dry aerial harvestable parts of the plant, yield of underground dry harvestable parts of the plant, yield of fresh weight harvestable parts of the plant, yield of aerial fresh weight harvestable parts of the plant yield of underground fresh weight harvestable parts of the plant, yield of the plant's fruit (both fresh and dried), grain dry weight, yield of seeds (both fresh and dry), and the like.

The most basic yield-related phenotype is increased yield associated with the presence of the gene or a homolog or a fragment thereof as a transgene in the plant, i.e., the intrinsic yield of the plant. Intrinsic yield capacity of a plant can be, for example, manifested in a field test or in a model system by demonstrating an improvement of seed yield (e.g. in terms of increased seed/grain size, increased ear number, increased seed number per ear, improvement of seed filling, improvement of seed composition, embryo and/or endosperm improvements, and the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under non-stressed conditions), improvement in plant architecture.

Increased yield-related phenotypes may also be measured to determine tolerance to abiotic environmental stress. Abiotic stresses include drought, low temperature, salinity, osmotic stress, shade, high plant density, mechanical stresses, and oxidative stress, and yield-related phenotypes are encompassed by tolerance to such abiotic stresses. Additional phenotypes that can be monitored to determine enhanced tolerance to abiotic environmental stress include, without limitation, wilting; leaf browning; loss of turgor, which results in drooping of leaves or needles stems, and flowers; drooping and/or shedding of leaves or needles; the leaves are green but leaf angled slightly toward the ground compared with controls; leaf blades begun to fold (curl) inward; premature senescence of leaves or needles; loss of chlorophyll in leaves or needles and/or yellowing. Any of the yield-related phenotypes described above may be monitored in field tests or in model plants to demonstrate that a transgenic plant has increased tolerance to abiotic environmental stress. In accordance with the invention, the respective genes identified in Table 1, in case in column 7 coenzyme Q10 and/or coenzyme Q9 is indicated, especially the coding region thereof, or homologs or fragments thereof, may be employed to enhance tolerance to abiotic environmental stress in a plant means that the plant, when confronted with abiotic environmental stress.

A polypeptide conferring an yield-increasing activity can be encoded by a respective nucleic acid sequence as shown in Table I, column 5 or 8, and/or comprises or consists of a respective polypeptide as depicted in Table II, column 5 and 8, and/or can be amplified with the respective primer set shown in Table III, column 8, in case in column 7 coenzyme Q10 and/or coenzyme Q9 is indicated.

“Improved adaptation” to environmental stress like e.g. freezing and/or chilling temperatures refers to an improved plant performance under environmental stress conditions.

A modification, i.e. an increase, can be caused by endogenous or exogenous factors. For example, an increase in activity in an organism or a part thereof can be caused by adding a gene product or a precursor or an activator or an agonist to the media or nutrition or can be caused by introducing said subjects into an organism, transient or stable. Furthermore such an increase can be reached by the introduction of the respective inventive nucleic acid sequence or the encoded protein in the correct cell compartment for example into the nucleus or cytoplasmic respectively or into plastids either by transformation and/or targeting.

The term “yield” as used herein generally refers to a measurable produce from a plant, particularly a crop. Yield and yield increase (in comparison to a non-transformed starting or wild-type plant) can be measured in a number of ways, and it is understood that a skilled person will be able to apply the correct meaning in view of the particular embodiments, the particular crop concerned and the specific purpose or application concerned. The terms “improved yield” or “increased yield” can be used interchangeable.

As used herein, the term “improved yield” or the term “increased yield” means any improvement in the yield of any measured plant product, such as grain, fruit or fiber. In accordance with the invention, changes in different phenotypic traits may improve yield. For example, and without limitation, parameters such as floral organ development, root initiation, root biomass, seed number, seed weight, harvest index, tolerance to abiotic environmental stress, leaf formation, phototropism, apical dominance, and fruit development, are suitable measurements of improved yield. Any increase in yield is an improved yield in accordance with the invention. For example, the improvement in yield can comprise a 0.1%, 0.5%, 1%, 3%, 5%, 10%, 15%, 20%, 30%, 40%, 50%, 60%, 70%, 80%, 90% or greater increase in any measured parameter. For example, an increase in the bushel/acre yield of soybeans or corn derived from a crop comprising plants which are transgenic for the nucleotides and polypeptides of Table I, in case in column 7 coenzyme Q10 and/or coenzyme Q9 is mentioned, as compared with the bushel/acre yield from untreated soybeans or corn cultivated under the same conditions, is an improved yield in accordance with the invention. The increased or improved yield can be achieved in the absence or presence of stress conditions.

For example, enhanced or increased “yield” refers to one or more yield parameters selected from the group consisting of biomass yield, dry biomass yield, aerial dry biomass yield, underground dry biomass yield, fresh-weight biomass yield, aerial fresh-weight biomass yield, underground fresh-weight biomass yield; enhanced yield of harvestable parts, either dry or fresh-weight or both, either aerial or underground or both; enhanced yield of crop fruit, either dry or fresh-weight or both, either aerial or underground or both; and preferably enhanced yield of seeds, either dry or fresh-weight or both, either aerial or underground or both.

Crop yield is defined herein as the number of bushels of relevant agricultural product (such as grain, forage, or seed) harvested per acre. Crop yield is impacted by abiotic stresses, such as drought, heat, salinity, and cold stress, and by the size (biomass) of the plant. Traditional plant breeding strategies are relatively slow and have in general not been successful in conferring increased tolerance to abiotic stresses. Grain yield improvements by conventional breeding have nearly reached a plateau in maize.

Accordingly, the yield of a plant can depend on the specific plant/crop of interest as well as its intended application (such as food production, feed production, processed food production, biofuel, biogas or alcohol production, or the like) of interest in each particular case. Thus, in one embodiment, yield is calculated as harvest index (expressed as a ratio of the weight of the respective harvestable parts divided by the total biomass), harvestable parts weight per area (acre, square meter, or the like); and the like. The harvest index, i.e., the ratio of yield biomass to the total cumulative biomass at harvest, in maize has remained essentially unchanged during selective breeding for grain yield over the last hundred years. Accordingly, recent yield improvements that have occurred in maize are the result of the increased total biomass production per unit land area. This increased total biomass has been achieved by increasing planting density, which has led to adaptive phenotypic alterations, such as a reduction in leaf angle, which may reduce shading of lower leaves, and tassel size, which may increase harvest index. Harvest index is relatively stable under many environmental conditions, and so a robust correlation between plant size and grain yield is possible. Plant size and grain yield are intrinsically linked, because the majority of grain biomass is dependent on current or stored photosynthetic productivity by the leaves and stem of the plant. As with abiotic stress tolerance, measurements of plant size in early development, under standardized conditions in a growth chamber or greenhouse, are standard practices to measure potential yield advantages conferred by the presence of a transgene.

Acccordingly, the yield of a plant can be increased by improving one or more of the yield-related phenotypes.

Such yield-related phenotypes or traits of a plant the improvement of which results in increased yield comprise, without limitation, the increase of the intrinsic yield capacity of a plant, improved nutrient use efficiency, and/or increased stress tolerance.

For example, yield refers to biomass yield, e.g. to dry weight biomass yield and/or fresh-weight biomass yield. Biomass yield refers to the aerial or underground parts of a plant, depending on the specific circumstances (test conditions, specific crop of interest, application of interest, and the like). In one embodiment, biomass yield refers to the aerial and underground parts. Biomass yield may be calculated as fresh-weight, dry weight or a moisture adjusted basis. Biomass yield may be calculated on a per plant basis or in relation to a specific area (e.g. biomass yield per acre/square meter/ or the like).

In other embodiment, “yield” refers to seed yield which can be measured by one or more of the following parameters: number of seeds or number of filled seeds (per plant or per area (acre/square meter/ or the like)); seed filling rate (ratio between number of filled seeds and total number of seeds); number of flowers per plant; seed biomass or total seeds weight (per plant or per area (acre/square meter/ or the like); thousand kernel weight (TKW; extrapolated from the number of filled seeds counted and their total weight; an increase in TKW may be caused by an increased seed size, an increased seed weight, an increased embryo size, and/or an increased endosperm). Other parameters allowing to measure seed yield are also known in the art. Seed yield may be determined on a dry weight or on a fresh weight basis, or typically on a moisture adjusted basis, e.g. at 15.5 percent moisture.

For example, the term “increased yield” means that the plant, exhibits an increased growth rate, under conditions of abiotic environmental stress, compared to the corresponding wild-type plant. An increased growth rate may be reflected inter alia by or confers an increased biomass production of the whole plant, or an increased biomass production of the aerial parts of a plant, or by an increased biomass production of the underground parts of a plant, or by an increased biomass production of parts of a plant, like stems, leaves, blossoms, fruits, and/or seeds.

Increased yield includes higher fruit yields, higher seed yields, higher fresh matter production, and/or higher dry matter production.

The term “increased yield” means that the plant, exhibits a prolonged growth under conditions of abiotic environmental stress, as compared to the corresponding, e.g. non-transformed, wild type organism. A prolonged growth comprises survival and/or continued growth of the plant, at the moment when the non-transformed wild type organism shows visual symptoms of deficiency and/or death.

When the plant of the invention is a corn plant increased yield for corn plants means, for example, increased seed yield, in particular for corn varieties used for feed or food. Increased seed yield of corn refers to an increased kernel size or weight, an increased kernel per ear, or increased ears per plant. Alternatively or in addition the cob yield may be increased, or the length or size of the cob is increased, or the kernel per cob ratio is improved.

When the plant of the invention is a soy plant, increased yield for soy plants means increased seed yield, in particular for soy varieties used for feed or food. Increased seed yield of soy refers for example to an increased kernel size or weight, an increased kernel per pod, or increased pods per plant.

When the plant of the invention is an oil seed rape (OSR) plant, increased yield for OSR plants means increased seed yield, in particular for OSR varieties used for feed or food. Increased seed yield of OSR refers to an increased seed size or weight, an increased seed number per silique, or increased siliques per plant.

When the plant of the invention is a cotton plant,increased yield for cotton plants means increased lint yield. Increased lint yield of cotton refers in one embodiment to an increased length of lint.

When the plant of the invention is a wheat plant increased yield for wheat plants means, for example, increased seed yield, in particular for wheat varieties used for feed or food. Increased seed yield of wheat refers to an increased kernel size or weight, an increased kernel per ear, or increased ears per plant. Increased seed yield of wheat refers also to an increased grain size or weight, an increased number of grains per spike, or increased number of spikes per plant. Alternatively or in addition increased seed yield of wheat refers also to an increased grain yield, increased length of spikes, increased number of spikelets per spike , increased 1000 grain weight or increased flag leaf area (flag leaf is the leaf directely below the spike).

Said increased yield can typically be achieved by enhancing or improving, one or more yield-related traits of the plant. Such yield-related traits of a plant comprise, without limitation, the increase of the intrinsic yield capacity of a plant, improved nutrient use efficiency, and/or increased stress tolerance, in particular increased abiotic stress tolerance.

Intrinsic yield capacity of a plant can be, for example, manifested by improving the specific (intrinsic) seed yield (e.g. in terms of increased seed/ grain size, increased ear number, increased seed number per ear, improvement of seed filling, improvement of seed composition, embryo and/or endosperm improvements, or the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under stressed or non-stressed conditions), improvement in plant architecture, cell cycle modifications, photosynthesis modifications, various signaling pathway modifications, modification of transcriptional regulation, modification of translational regulation, modification of enzyme activities, and the like); and/or the like.

The improvement or increase of stress tolerance of a plant can for example be manifested by improving or increasing a plant's tolerance against stress, particularly abiotic stress. In the present application, abiotic stress refers generally to abiotic environmental conditions a plant is typically confronted with, including, but not limited to, drought (tolerance to drought may be achieved as a result of improved water use efficiency), heat, low temperatures and cold conditions (such as freezing and chilling conditions), salinity, osmotic stress , shade, high plant density, mechanical stress, oxidative stress, and the like.

The increased plant yield can also be mediated by increasing the “nutrient use efficiency of a plant”, e.g. by improving the use efficiency of nutrients including, but not limited to, phosphorus, potassium, and nitrogen. Further, higher yields may be obtained with current or standard levels of nitrogen use.

Generally, the term “increased tolerance to stress” can be defined as survival of plants, and/or higher yield production, under stress conditions as compared to a non-transformed wild type or starting plant: For example, the plant of the invention or produced according to the method of the invention is better adapted to the stress conditions. “Improved adaptation” to environmental stress like e.g. drought, heat, nutrient depletion, freezing and/or chilling temperatures refers herein to an improved plant performance resulting in an increased yield, particularly with regard to one or more of the yield related traits as defined in more detail above. During its life-cycle, a plant is generally confronted with a diversity of environmental conditions. Any such conditions, which may, under certain circumstances, have an impact on plant yield, are herein referred to as “stress” condition. Environmental stresses may generally be divided into biotic and abiotic (environmental) stresses. Unfavorable nutrient conditions are sometimes also referred to as “environmental stress”. The present invention does also contemplate solutions for this kind of environmental stress, e.g. referring to increased nutrient use efficiency. For the purposes of the description of the present invention, the terms “enhanced tolerance to abiotic stress”, “enhanced resistance to abiotic environmental stress”, “enhanced tolerance to environmental stress”, “improved adaptation to environmental stress” and other variations and expressions similar in its meaning are used interchangeably and refer, without limitation, to an improvement in tolerance to one or more abiotic environmental stress(es) as described herein and as compared to a corresponding origin or wild type plant or a part thereof.

The term abiotic stress tolerance(s) refers for example low temperature tolerance, drought tolerance or improved water use efficiency (WUE), heat tolerance, salt stress tolerance and others. Studies of a plant's response to desiccation, osmotic shock, and temperature extremes are also employed to determine the plant's tolerance or resistance to abiotic stresses. Water use efficiency (WUE) is a parameter often correlated with drought tolerance. In selecting traits for improving crops, a decrease in water use, without a change in growth would have particular merit in an irrigated agricultural system where the water input costs were high. An increase in growth without a corresponding jump in water use would have applicability to all agricultural systems. In many agricultural systems where water supply is not limiting, an increase in growth, even if it came at the expense of an increase in water use also increases yield.

Drought stress means any environmental stress which leads to a lack of water in plants or reduction of water supply to plants, including a secondary stress by low temperature and/or salt, and/or a primary stress during drought or heat, e.g. desiccation etc.

### Specific Embodiments

Accordingly, this invention provides respective measures and methods to produce plants with increased yield, e.g. genes conferring an increased yield-related trait, for example enhanced tolerance to abiotic environmental stress, for example an increased drought tolerance and/or low temperature tolerance and/or an increased nutrient use efficiency, intrinsic yield and/or another increased yield-related trait, upon expression or over-expression. Accordingly, the present invention provides such genes in case in column 7 of the respective Table or coenzyme Q10 and/or coenzyme Q9 is indicated. In particular, such genes are described in column 5 as well as in column 8 of Tables I, especially the coding region thereof, or homologs or fragments thereof, in case coenzyme Q10 and/or coenzyme Q9 are indicated in column 7 or the respective polypeptides are described in column 5 as well as in column 8 of Table II, or homologs or fragments thereof, in case coenzyme Q10 and/or coenzyme Q9 are indicated in column 7.

Accordingly, the present invention provides respective transgenic plants showing one or more improved yield-related traits as compared to the corresponding origin or the wild type plant and methods for producing such transgenic plants with increased yield in case in column 7 of the respective Table “coenzyme Q10” or “coenzyme Q9” is indicated.

In one embodiment, one or more of said yield-increasing activities are increased by increasing the amount and/or the specific activity of one or more proteins listed in Table II, column 5 or 8 in a compartment of a cell indicated in Table II, column 6, in case in column 7 of the respective Table “coenzyme Q10” or “coenzyme Q9” is indicated. Accordingly to present invention, the yield of the plant of the invention is increased by improving one or more of the yield-related traits as defined herein. Said increased yield in accordance with the present invention can typically be achieved by enhancing or improving, in comparison to an origin or wild-type plant, one or more yield-related traits of said plant. Such yield-related traits of a plant the improvement of which results in increased yield comprise, without limitation, the increase of the intrinsic yield capacity of a plant, improved nutrient use efficiency, like nitrogen use efficiency, and/or increased stress tolerance.

In one embodiment, the present invention provides respective transgenic plants showing one or more improved yield-related traits, preferably increased drought tolerance as compared to the corresponding origin or the wild type plant and methods for producing such transgenic plants with increased yield in case in column 7 of the respective Table “coenzyme Q10” or “coenzyme Q9” is indicated.

In a further embodiment the present invention is characterized by the features as disclosed in the following items:

Item 1. A process for the production of a fine chemical selected from the group consisting of: coenzyme Q10, coenzyme Q9, lipoic acid and/or nicotinic acid
(a) increasing or generating one or more activities selected from the group consisting of 2-C-methyl-D-erythritol 4-phosphate cytidylyltransferase, acetolactate synthase III large subunit, acetyltransferase, acid shock protein, amino acid ABC transporter permease protein, amino acid acetyltransferase, At1g47380-protein, ATP synthase subunit E, ATP-binding component of a transport system, auxin response factor, AX653549-protein, AY087308-protein, b1003-protein, b1097-protein, b1522-protein, b2032-protein, b2739-protein, b2849-protein, b3817-protein, b4029-protein, beta-galactosidase, calcium-dependent protein kinase, cation-transporting ATPase, CCAAT-binding transcription factor, cell division control protein, colanic acid biosynthesis protein, CTP synthase, cyclin D , disease resistance protein, enoyl-CoA hydratase, fatty acid desaturase, F-box protein, flavodoxin, gluconate transport system permease 3, glutamine amidotransferase, glutaredoxin, glycerol dehydrogenase, glycerol-3-phosphate dehydrogenase, glycosyl transferase, halotolerance protein, heat shock protein Hsp40, heat shock transcription factor, histone acetyltransferase, hydrolase, isopropylmalate isomerase large subunit, L-aspartate oxidase, lipase, MADS box protein transcription factor, malate dehydrogenase, methylglyoxal synthase, methyltransferase, mitogen-activated protein kinase, monothiol glutaredoxin, multiple antibiotic resistance protein, N-acetyl-gamma-glutamylphosphate reductase, NADH dehydrogenase I chain I, oxidoreductase, permease protein of ABC transporter, phenylalanine ammonia-lyase, phosphoadenosine phosphosulfate reductase, phosphopantothenoylcysteine decarboxylase, photosystem II protein, protein kinase, protein phosphatase, pyruvate kinase, recombinase A, Ribose import ATP-binding protein, Sec-independent protein translocase subunit, serine protease, serine/threonine-protein phosphatase, sll1761-protein, slr1107-protein, sterol desaturase, threonine synthase, transcription factor, transcriptional regulator, TTC1386-protein, uroporphyrin-III C-methyltransferase, ycl027w-protein, zinc finger protein and zinc transporter, in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of coenzyme Q10, coenzyme Q9, lipoic acid and/or nicotinic acid or a composition comprising coenzyme Q10, coenzyme Q9, lipoic acid and/or nicotinic acid in said non-human organism or in the culture medium surrounding said non-human organism.

Item 2. A process for the production of a a fine chemical selected from the group consisting of: coenzyme Q10, coenzyme Q9, lipoic acid and/or nicotinic acid, which comprises
(A)
   (i) increasing or generating of the expression of; and/or
   (ii) increasing or generating the expression of an expression product of; and/or
   (iii) increasing or generating one or more activities of an expression product encoded by;
   at least one respective nucleic acid molecule comprising a nucleic acid molecule selected from the group consisting of:
   (a) a nucleic acid molecule encoding the polypeptide shown in column 5 or 8 of Table II, preferably Table II B, application no.16, or a homolog or a fragment thereof;
   (b) a nucleic acid molecule shown in column 5 or 8 of Table I, preferably Table I B, application no.16, or a homolog or a fragment thereof (preferably the coding region thereof);
   (c) a nucleic acid molecule, which, as a result of the degeneracy of the genetic code, can be derived from a polypeptide sequence depicted in column 5 or 8 of Table II, preferably Table II B, application no.16;
   (d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 16, or the coding region thereof;
   (e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no.16;
   (i) a nucleic acid molecule encoding a polypeptide having the activity represented by a respective protein as depicted in column 5 of Table II, application no. 16;
   (j) a nucleic acid molecule which comprises a polynucleotide, which is obtained by amplifying a cDNA library or a genomic library using the primers as depicted in column 8 of Table III, application no. 16; and
   (k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
   or a nucleic acid molecule comprising a sequence which is complementary thereto; in a non-human organism or a part thereof, preferably a microorganism, a plant cell, a plant or a part thereof, as compared to a corresponding non-transformed wild type non-human organism or a part thereof; and
(B) growing the non-human organism or a part thereof under conditions which permit the production of coenzyme Q10, coenzyme Q9, lipoic acid and/or nicotinic acid or a composition comprising coenzyme Q10, coenzyme Q9, lipoic acid and/or nicotinic acid in said non-human organism or in the culture medium surrounding said non-human organism.

Item 3. A process of items 1 or 2, comprising of recovering coenzyme Q10, coenzyme Q9, lipoic acid and/or nicotinic acid in its free or bound form.

Item 4. The process of any one of items 1 to 3, comprising the following steps:
(a) selecting a non-human organism or a part thereof expressing a polypeptide encoded by the nucleic acid molecule characterized in item 2;
(b) mutagenizing the selected non-human organism or the part thereof;
(c) comparing the activity or the expression level of said polypeptide in the mutagenized non-human organism or the part thereof with the activity or the expression of said polypeptide of the selected non-human organisms or the part thereof;
(d) selecting the mutated non-human organisms or parts thereof, which comprise an increased activity or expression level of said polypeptide compared to the selected non-human organism or the part thereof;
(e) optionally, growing and cultivating the non-human organisms or the parts thereof; and
(f) recovering, and optionally isolating, the free or bound coenzyme Q10, coenzyme Q9, lipoic acid and/or nicotinic acid produced by the selected mutated non-human organisms or parts thereof.

Item 5. The process of any one of items 1 to 4 wherein the activity of said protein or the expression of said nucleic acid molecule is increased or generated transiently or stably.

Item 6. An isolated nucleic acid molecule comprising a nucleic acid molecule selected from the group consisting of:
(a) a nucleic acid molecule encoding a polypeptide shown in Table II, application no. 16, column 5 or 8, preferably shown in Table II B, application no. 16, column 8, or a homolog or a fragment thereof;
(b) a nucleic acid molecule shown in Table I, application no. 16, column 5 or 8, preferably shown in Table I B, application no. 16, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 16, preferably in column 8 of Table II B, application no. 16;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 16, preferably shown in column 8 of Table I B, application no. 16, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 16;
(i) a nucleic acid molecule encoding a polypeptide having the activity represented by a respective protein as depicted in column 5 of Table II, application no. 16;
(j) a nucleic acid molecule comprising a polynucleotide which is obtained by amplifying a cDNA library or a genomic library using the primers in Table III, application no. 16, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (i);
or a nucleic acid molecule comprising a sequence which is complementary thereto.

Item 7. A nucleic acid construct which confers the expression of the nucleic acid molecule as defined in item 6, comprising one or more regulatory elements.

Item 8. An expression cassette comprising
(a) a promoter, preferably selected from the group consisting of Big35S, PCUbi, Super and USP;
(b) a nucleic acid molecule as defined in item 6.

Item 9. A vector comprising the nucleic acid molecule as defined in item 6 or the nucleic acid construct as claimed in item 7 or the expression cassette as claimed in item 8.

Item 10. A host cell or a plant organelle, which has been transformed with the vector as claimed in item 9 or the nucleic acid molecule as defined in item 6 or the nucleic acid construct as claimed in item 7 or the expression cassette as claimed in item 8.

Item 11. The host cell of item 10, which is a plant cell or a microorganism.

Item 12. A process for producing a polypeptide, wherein the polypeptide is expressed in a host cell as claimed in item 10 or 11.

Item 13. A polypeptide produced by the process as claimed in item 12 or encoded by the nucleic acid molecule as defined in item 6.

Item 14. An antibody, which binds specifically to the polypeptide as claimed in item 13.

Item 15. A plant tissue, propagation material, harvested material or a plant comprising the host cell as claimed in item 10 or 11.

Item 16. A process for the identification of a compound conferring an increase in coenzyme Q10, coenzyme Q9, lipoic acid and/or nicotinic acid production in a non-human organism, comprising the steps:
(a) culturing a plant cell or tissue or microorganism or maintaining a plant expressing the polypeptide encoded by the nucleic acid molecule as defined in item 6 conferring an increase in the amount of coenzyme Q10, coenzyme Q9, lipoic acid and/or nicotinic acid in a non-human organism or a part thereof and a readout system capable of interacting with the polypeptide under suitable conditions which permit the interaction of the polypeptide with said readout system in the presence of a compound or a sample comprising a plurality of compounds and capable of providing a detectable signal in response to the binding of a compound to said polypeptide under conditions which permit the expression of said readout system and of the polypeptide encoded by the nucleic acid molecule of item 6 conferring an increase in the amount of coenzyme Q10, coenzyme Q9, lipoic acid and/or nicotinic acidin a non-human organism or a part thereof;
(b) identifying if the compound is an effective agonist by detecting the presence or absence or increase of a signal produced by said readout system.

Item 17. A method for the production of an agricultural composition comprising the steps of the method of item 16 and formulating the compound identified in item 16 in a form acceptable for an application in agriculture.

Item 18. A composition comprising the nucleic acid molecule as defined in item 6, the polypeptide as claimed in item 13, the nucleic acid construct as claimed in item 7, the expression cassette as claimed in item 8, the vector as claimed in item 9, the compound as claimed in item 16, the antibody as claimed in item 14, and optionally an agricultural acceptable carrier.

Item 19. Use of the nucleic acid molecule as defined in item 6 for the identification of a nucleic acid molecule conferring an increase in coenzyme Q10, coenzyme Q9, lipoic acid and/or nicotinic acid after expression.

Item 20. Cosmetic, pharmaceutical, food or feed composition comprising the nucleic acid moleculeas defined in item 6, the polypeptide as claimed in item 13, the nucleic acid construct as claimed in item 7, the expression cassette as claimed in item 8, the vector as claimed in item 9, the antibody as claimed in item 14 , the plant or plant tissue as claimed in item 15, the harvested material as claimed in item 15 or the host cell of item as claimed in item s 10 or 11.

Item 21. Use of the nuclei acid molecule as defined in item 6, the polypeptide as claimed in item 13, the nucleic acid construct as claimed in item 7, the expression cassette as claimed in item 8, the vector as claimed in item 9, the plant or plant tissue as claimed in item 15, or the host cell of item 10 to 11 for the production of plant resistant to a herbicide inhibiting the production of coenzyme Q10, coenzyme Q9, lipoic acid and/or nicotinic acid.

Item 22. A method for producing a plant with increased yield as compared to a corresponding non-transformed wild type plant, whereby the method comprises
(i) increasing or generating of the expression of; and/or
(ii) increasing or generating the expression of an expression product of; and/or
(iii) increasing or generating one or more activities of an expression product encoded by; at least one respective nucleic acid molecule comprising a nucleic acid molecule selected from the group consisting of:
(a) a nucleic acid molecule encoding the polypeptide shown in column 5 or 8 of Table II, preferably Table II B, application no. 16, or a homolog or a fragment thereof, in case in column 7 coenzyme Q10 and/or coenzyme Q9 is indicated;
(b) a nucleic acid molecule shown in column 5 or 8 of Table I, preferably Table I B, application no. 16, or a homolog or a fragment thereof (preferably the coding region thereof) , in case in column 7 coenzyme Q10 and/or coenzyme Q9 is indicated;
(c) a nucleic acid molecule, which, as a result of the degeneracy of the genetic code, can be derived from a polypeptide sequence depicted in column 5 or 8 of Table II, preferably Table II B, application no. 16, in case in column 7 coenzyme Q10 and/or coenzyme Q9 is indicated;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 16, or the coding region thereof, in case in column 7 coenzyme Q10 and/or coenzyme Q9 is indicated;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 16, in case in column 7 coenzyme Q10 and/or coenzyme Q9 is indicated;
(i) a nucleic acid molecule encoding a polypeptide having the activity represented by a respective protein as depicted in column 5 of Table II, application no. 16, in case in column 7 coenzyme Q10 and/or coenzyme Q9 is indicated;
(j) a nucleic acid molecule which comprises a polynucleotide, which is obtained by amplifying a cDNA library or a genomic library using the primers as depicted in column 8 of Table III, application no. 16, in case in column 7 coenzyme Q10 and/or coenzyme Q9 is indicated; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto; in a plant cell, plant or a part thereof.

Item 23. A method for producing a plant with increased yield according to item 22, which comprises in addition the regeneration of a plant from such transformed plant cell, plant cell compartment, plant cell nucleus, plant cell or plant tissue with increased yield.

[0483.1.7.16] to [0492.1.7.16] for the disclosure of these paragraphs see [0483.1.7.7] to [0492.1.7.7] above

### Example 10

### Purification of a Fine Chemical e.g. Coenzyme Q10, Coenzyme Q9, Lipoic Acid and/or Nicotinic Acid

Cofactors can be recovered from cells or from the supernatant of the above-described culture by a variety of methods known in the art. For example, the culture supernatant is recovered first. To this end, the cells are harvested from the culture by slow centrifugation.

The cell debris is removed by centrifugation and the supernatant fraction, if appropriate together with the culture supernatant, is used for the further purification of the cofactors. However, it is also possible to process the supernatant alone if the cofactors are present in the supernatant in sufficiently high a concentration. In this case, the cofactors, or the cofactors mixture, can be purified further for example via extraction and/or salt precipitation or via ion-exchange chromatography or as described above.

Identity and purity of the isolated cofactors such as coenzyme Q10, coenzyme Q9, lipoic acid and/or nicotinic acid can be determined by standard techniques of the art. They encompass old techniques such as photometric methods and new techniques such as high-performance liquid chromatography (HPLC), Reversed phase chromatographie and TLC.

for the disclosure of this paragraph see [0494.1.7.7] above

### Example 11a

### PCR Amplification of the Sequences

Unless otherwise specified, standard methods as described in Sambrook et al., Molecular Cloning:

A laboratory manual, Cold Spring Harbor 1989, Cold Spring Harbor Laboratory Press are used.

The inventive sequences as shown in the respective line in Table I, column 5, preferably the coding region thereof, were amplified by PCR as described in the protocol of the Pfu Ultra, Pfu Turbo or Herculase DNA polymerase (Stratagene). The composition for the protocol of the Pfu Ultra, Pfu Turbo or Herculase DNA polymerase was as follows: 1×PCR buffer (Stratagene), 0.2 mM of each dNTP, 100 ng genomic DNA of Saccharomyces cerevisiae (strain S288C; Research Genetics, Inc., now Invitrogen), Escherichia coli (strain MG1655; E. coli Genetic Stock Center), Synechocystis sp. (strain PCC6803), Azotobacter vinelandii (strain N.R. Smith,16), Thermus thermophilus (HB8) or 50 ng cDNA from various tissues and development stages of Arabidopsis thaliana (ecotype Columbia), Physcomitrella patens, Glycine max (variety Resnick), Brassica napus, Oryza sativa or Zea mays (variety B73, Mo17, A188), 50 pmol forward primer, 50 pmol reverse primer, with or without 1 M Betaine, 2.5 u Pfu Ultra, Pfu Turbo or Herculase DNA polymerase.

The amplification cycles were as follows:

1 cycle of 2-3 minutes at 94-95° C., then 25-36 cycles with 30-60 seconds at 94-95° C., 30-45 seconds at 50-60° C. and 210-480 seconds at 72° C., followed by 1 cycle of 5-10 minutes at 72° C., then 4-16° C.—preferably for Saccharomyces cerevisiae, Escherichia coli, Synechocystis sp., Azotobacter vinelandii, Thermus thermophilus.

In case of Arabidopsis thaliana, Brassica napus, Glycine max, Oryza sativa, Physcomitrella patens, Zea mays the amplification cycles were as follows: 1 cycle with 30 seconds at 94° C., 30 seconds at 61° C., 15 minutes at 72° C., then 2 cycles with 30 seconds at 94° C., 30 seconds at 60° C., 15 minutes at 72° C., then 3 cycles with 30 seconds at 94° C., 30 seconds at 59° C., 15 minutes at 72° C., then 4 cycles with 30 seconds at 94° C., 30 seconds at 58° C., 15 minutes at 72° C., then 25 cycles with 30 seconds at 94° C., 30 seconds at 57° C., 15 minutes at 72° C., then 1 cycle with 10 minutes at 72° C., then finally 4-16° C.

RNAs were generated with the RNeasy Plant Kit according to the standard protocol (Qiagen) and Superscript II Reverse Transkriptase was used to produce double stranded cDNA according to the standard protocol (Invitrogen).

ORF specific primer pairs for the genes to be expressed are shown in the respective line in Table III, column 8. The following adapter sequences were added to Saccharomyces cerevisiae

ORF specific primers (see Table III) for cloning purposes:

SEQ ID NO: 20

| | | |
|---|---|---|
| i) | foward primer: | 5′-GGAATTCCAGCTGACCACC-3′ |

SEQ ID NO: 21

| | | |
|---|---|---|
| ii) | reverse primer: | 5′-GATCCCCGGGAATTGCCATG-3′ |

These adaptor sequences allow cloning of the ORF into the various vectors containing the Resgen adaptors, see table column E of Table c.

The following adapter sequences were added to Saccharomyces cerevisiae, Escherichia coli, Synechocystis sp., Azotobacter vinelandii, Thermus thermophilus, Arabidopsis thaliana, Brassica napus, Glycine max, Oryza sativa , Physcomitrella patens, or Zea mays ORF specific primers for cloning purposes:

| SEQ ID NO: 22 | | |
|---|---|---|
| iii) | forward primer: | 5′-TTGCTCTTCC-3′ |
| SEQ ID NO: 23 | | |
| iiii) | reverse primer: | 5′-TTGCTCTTCG-3′ |

The adaptor sequences allow cloning of the ORF into the various vectors containing the Colic adaptors, see table column E of Table c.

Therefore for amplification and cloning of Saccharomyces cerevisiae SEQ ID NO: 62717, a primer consisting of the adaptor sequence i) and the ORF specific sequence SEQ ID NO: 63159 and a second primer consisting of the adaptor sequence ii) and the ORF specific sequence SEQ ID NO: 63160 were used.

For amplification and cloning of Escherichia coli SEQ ID NO: 7081, a primer consisting of the adaptor sequence iii) and the ORF specific sequence SEQ ID NO: 7261 and a second primer consisting of the adaptor sequence iiii) and the ORF specific sequence SEQ ID NO: 7262 were used.

For amplification and cloning of Synechocystis sp. SEQ ID NO: 11990, a primer consisting of the adaptor sequence iii) and the ORF specific sequence SEQ ID NO: 12064 and a second primer consisting of the adaptor sequence iiii) and the ORF specific sequence SEQ ID NO: 12065 were used.

For amplification and cloning of Arabidopsis thaliana SEQ ID NO: 2367, a primer consisting of the adaptor sequence iii) and the ORF specific sequence SEQ ID NO: 2561 and a second primer consisting of the adaptor sequence iiii) and the ORF specific sequence SEQ ID NO: 2562 were used.

For amplification and cloning of Azotobacter vinelandii SEQ ID NO: 6510, a primer consisting of the adaptor sequence iii) and the ORF specific sequence SEQ ID NO: 6670 and a second primer consisting of the adaptor sequence iiii) and the ORF specific sequence SEQ ID NO: 6671 were used.

For amplification and cloning of Glycine max SEQ ID NO: 46751, a primer consisting of the adaptor sequence iii) and the ORF specific sequence SEQ ID NO: 46841 and a second primer consisting of the adaptor sequence iiii) and the ORF specific sequence SEQ ID NO: 46842 were used.

For amplification and cloning of Oryza sativa SEQ ID NO: 34301, a primer consisting of the adaptor sequence iii) and the ORF specific sequence SEQ ID NO: 34595 and a second primer consisting of the adaptor sequence iiii) and the ORF specific sequence SEQ ID NO: 34596 were used.

For amplification and cloning of Thermus thermophilus SEQ ID NO: 12698, a primer consisting of the adaptor sequence iii) and the ORF specific sequence SEQ ID NO: 12970 and a second primer consisting of the adaptor sequence iiii) and the ORF specific sequence SEQ ID NO: 12971 were used.

For amplification and cloning of Zea mays SEQ ID NO: 68132, a primer consisting of the adaptor sequence iii) and the ORF specific sequence SEQ ID NO: 68358 and a second primer consisting of the adaptor sequence iiii) and the ORF specific sequence SEQ ID NO: 68359 were used.

Following these examples every sequence disclosed in Table I, preferably column 5, especially the coding region thereof can be cloned by fusing the adaptor sequences to the respective specific primers sequences as disclosed in Table III, column 8 using the respective vectors shown in Table c.

[0496.1.7.16] to [0499.1.7.16] for the disclosure of theses paragraphs see [0496.1.7.7] to [0499.1.7.7] above

### Example 11e

### Cloning of Inventive Sequences as Shown in Table I, Column 5 in the Different Expression Vectors

For cloning of for example SEQ ID NO: 7081from Escherichia coli or any other ORF from Escherichia coli, Synechocystis sp., Azotobacter vinelandii, Thermus thermophilus, Arabidopsis thaliana, Glycine max, Oryza sativa , or Zea mays the vector DNA was treated with the restriction enzymes Pacl and Ncol following the standard protocol (MBI Fermentas).

For cloning for example the ORFs of SEQ ID NO: 62717 from Saccharomyces cerevisiae or any other ORF from Saccharomyces cerevisiae into vectors containing the Resgen adaptor sequence the respective vector DNA was treated with the restriction enzyme Ncol.

For cloning of for example ORFs SEQ ID NO: 102061 from Saccharomyces cerevisiae or any other ORF from Saccharomyces cerevisiae into vectors containing the Colic adaptor sequence, the respective vector DNA was treated with the restriction enzymes Pacl and Ncol following the standard protocol (MBI Fermentas).

In all cases the reaction was stopped by inactivation at 70° C. for 20 minutes and purified over QlAquick or NucleoSpin Extract II columns following the standard protocol (Qiagen or Macherey-Nagel).

Then the PCR-product representing the amplified ORF with the respective adapter sequences and the vector DNA were treated with T4 DNA polymerase according to the standard protocol (MBI Fermentas) to produce single stranded overhangs with the parameters 1 unit T4 DNA polymerase at 37° C. for 2-10 minutes for the vector and 1-2 u T4 DNA polymerase at 15-17° C. for 10-60 minutes for the PCR product representing SEQ ID NO: 7081.

The reaction was stopped by addition of high-salt buffer and purified over QlAquick or NucleoSpin Extract II columns following the standard protocol (Qiagen or Macherey-Nagel).

According to this example the skilled person is able to clone all sequences disclosed in Table I, preferably column 5 or column 8, especially the coding region thereof.

Approximately 30-60 ng of prepared vector and a defined amount of prepared amplificate were mixed and hybridized at 65° C. for 15 minutes followed by 37° C. 0.1° C/1 seconds, followed by 37° C. 10 minutes, followed by 0.1° C/1 seconds, then 4-10 ° C. The ligated constructs were transformed in the same reaction vessel by addition of competent E. coli cells (strain DH5-alpha) and incubation for 20 minutes at 1 ° C. followed by a heat shock for 90 seconds at 42° C. and cooling to 1-4° C. Then, complete medium (SOC) was added and the mixture was incubated for 45 minutes at 37° C. The entire mixture was subsequently plated onto an agar plate with 0.05 mg/ml kanamycin and incubated overnight at 37° C.

The outcome of the cloning step was verified by amplification with the aid of primers which bind upstream and downstream of the integration site, thus allowing the amplification of the insertion.

The amplifications were carried out as described in the protocol of Taq DNA polymerase (Gibco-BRL).

The amplification cycles were as follows:

1 cycle of 1-5 minutes at 94° C., followed by 35 cycles of in each case 15-60 seconds at 94° C., 15-60 seconds at 50-66° C. and 5-15 minutes at 72° C., followed by 1 cycle of 10 minutes at 72° C., then 4-16° C.

Several colonies were checked, but only one colony for which a PCR product of the expected size was detected was used in the following steps.

A portion of this positive colony was transferred into a reaction vessel filled with complete medium (LB) supplemented with kanamycin and incubated overnight at 37° C.

The plasmid preparation was carried out as specified in the Qiaprep or NucleoSpin Multi-96 Plus standard protocol (Qiagen or Macherey-Nagel).

[0501.1.7.16] to [0503.1.7.16] for the disclosure of these paragraphs see [0501.1.7.7] to [0503.1.7.7] above for the disclosure of this paragraph see above

for the disclosure of this paragraph see [0502.1.7.7] above

Table d showing results of plant analyses

TABLE d

| SeqID | Target | Locus | Metabolite | Source | Promotor | Method | Min | Max |
|---|---|---|---|---|---|---|---|---|
| 17356 | plastidic | AAC43185 | Co Q10 | ARA_LEAF | p-PcUBI | LC | 67 | 296 |
| 18070 | non-targ | At1g43850 | Co Q9 | ARA_LEAF | p-PcUBI | LC | 37 | 123 |
| 18122 | non-targ | At1g47380 | Co Q9 | ARA_LEAF | p-PcUBI | LC | 36 | 98 |
| 19419 | non-targ | At1g72770 | Co Q9 | ARA_LEAF | p-PcUBI | LC | 52 | 65 |
| 133042 | non-targ | At2g37040 | Co Q9 | ARA_LEAF | p-PcUBI | LC | 39 | 85 |
| 108727 | non-targ | At2g42830 | Co Q9 | ARA_LEAF | p-PcUBI | LC | 37 | 91 |
| 21159 | non-targ | At2g47880 | Co Q10 | ARA_LEAF | p-PcUBI | LC | 79 | 87 |
| 68657 | non-targ | At3g02990 | Co Q9 | ARA_LEAF | p-PcUBI | LC | 40 | 148 |
| 21497 | plastidic | At3g04050 | Co Q10 | ARA_LEAF | p-PcUBI | LC | 85 | 142 |
| 120952 | non-targ | At3g16820 | Co Q9 | ARA_LEAF | p-PcUBI | LC | 45 | 114 |
| 22921 | non-targ | At3g20910 | Co Q10 | ARA_LEAF | p-PcUBI | LC | 67 | 438 |
| 22921 | non-targ | At3g20910 | Co Q9 | ARA_LEAF | p-PcUBI | LC | 37 | 70 |
| 68777 | non-targ | At3g27540 | Co Q9 | ARA_LEAF | p-PcUBI | LC | 36 | 86 |
| 2367 | non-targ | At3g61830 | Co Q9 | ARA_LEAF | p-PcUBI | LC | 36 | 177 |
| 2935 | non-targ | At3g62950 | Co Q10 | ARA_LEAF | p-PcUBI | LC | 52 | 102 |
| 2935 | non-targ | At3g62950 | Co Q9 | ARA_LEAF | p-PcUBI | LC | 52 | 91 |
| 131208 | non-targ | At4g12110 | Co Q10 | ARA_LEAF | p-PcUBI | LC | 72 | 96 |
| 23482 | non-targ | At4g15660 | Co Q10 | ARA_LEAF | p-PcUBI | LC | 89 | 174 |
| 23482 | non-targ | At4g15660 | Co Q9 | ARA_LEAF | p-PcUBI | LC | 53 | 105 |
| 24311 | non-targ | At4g34160 | Co Q10 | ARA_LEAF | p-PcUBI | LC | 78 | 175 |
| 24311 | non-targ | At4g34160 | Co Q9 | ARA_LEAF | p-PcUBI | LC | 38 | 131 |
| 4348 | non-targ | At4g35310 | Co Q10 | ARA_LEAF | p-PcUBI | LC | 72 | 393 |
| 4348 | non-targ | At4g35310 | Co Q9 | ARA_LEAF | p-PcUBI | LC | 36 | 244 |
| 24438 | non-targ | At5g03720 | Co Q9 | ARA_LEAF | p-PcUBI | LC | 46 | 53 |
| 4904 | non-targ | At5g18600 | Co Q10 | ARA_LEAF | p-PcUBI | LC | 57 | 63 |
| 102223 | non-targ | At5g45060 | Co Q9 | ARA_LEAF | p-PcUBI | LC | 41 | 53 |
| 25498 | non-targ | At5g59220 | Co Q9 | ARA_LEAF | p-PcUBI | LC | 36 | 97 |
| 5493 | non-targ | At5g64920 | Co Q9 | ARA_LEAF | p-PcUBI | LC | 36 | 114 |
| 29286 | non-targ | Avin- | Co Q9 | ARA_LEAF | p-PcUBI | LC | 52 | 101 |
| | | DRAFT_3209 | | | | | | |
| 31026 | non-targ | Avin- | Co Q9 | ARA_LEAF | p-PcUBI | LC | 46 | 176 |
| | | DRAFT_3605 | | | | | | |
| 114231 | non-targ | Avin- | Co Q9 | ARA_LEAF | p-PcUBI | LC | 42 | 171 |
| | | DRAFT_3629 | | | | | | |
| 73038 | non-targ | Avin- | Co Q10 | ARA_LEAF | p-PcUBI | LC | 109 | 257 |
| | | DRAFT_4420 | | | | | | |
| 73038 | non-targ | Avin- | Co Q9 | ARA_LEAF | p-PcUBI | LC | 98 | 220 |
| | | DRAFT_4420 | | | | | | |
| 102941 | non-targ | Avin- | Co Q9 | ARA_LEAF | p-PcUBI | LC | 36 | 79 |
| | | DRAFT_4847 | | | | | | |
| 6510 | non-targ | Avin- | Co Q9 | ARA_LEAF | p-PcUBI | LC | 195 | 240 |
| | | DRAFT_5103 | | | | | | |
| 34301 | non-targ | AX653549 | Co Q9 | ARA_LEAF | p-PcUBI | LC | 44 | 55 |
| 34602 | non-targ | AY087308 | Co Q10 | ARA_LEAF | p-PcUBI | LC | 60 | 92 |
| 34889 | plastidic | B0004 | Co Q10 | ARA_LEAF | p-Super | LC | 70 | 558 |
| 137834 | plastidic | B0077 | Co Q10 | ARA_SEED_2 | p-USP | LC | 21 | 34 |
| 7081 | non-targ | B0161 | Co Q9 | ARA_LEAF | p-Super | LC | 38 | 146 |
| 35733 | plastidic | B0344 | Co Q9 | ARA_SEED_2 | p-USP | LC | 17 | 59 |
| 7333 | non-targ | B0449 | Co Q10 | ARA_LEAF | p-Super | LC | 69 | 266 |
| 7333 | non-targ | B0449 | Co Q9 | ARA_LEAF | p-Super | LC | 71 | 209 |
| 36114 | non-targ | B0752 | Co Q10 | ARA_LEAF | p-Super | LC | 98 | 188 |
| 36114 | non-targ | B0752 | Co Q9 | ARA_LEAF | p-Super | LC | 53 | 96 |
| 36670 | non-targ | B0963 | Co Q10 | ARA_LEAF | p-Super | LC | 54 | 113 |
| 36670 | non-targ | B0963 | Co Q9 | ARA_LEAF | p-Super | LC | 46 | 60 |
| 7941 | non-targ | B1003 | Co Q9 | ARA_LEAF | p-Super | LC | 40 | 287 |
| 138256 | non-targ | B1097 | Co Q9 | ARA_LEAF | p-Super | LC | 40 | 68 |
| 7947 | non-targ | B1522 | Co Q10 | ARA_LEAF | p-Super | LC | 56 | 198 |
| 7947 | non-targ | B1522 | Co Q9 | ARA_LEAF | p-Super | LC | 36 | 144 |
| 38300 | non-targ | B1597 | Co Q10 | ARA_LEAF | p-Super | LC | 81 | 159 |
| 38300 | non-targ | B1597 | Co Q9 | ARA_LEAF | p-Super | LC | 46 | 168 |
| 8316 | non-targ | B1838 | Co Q9 | ARA_LEAF | p-Super | LC | 36 | 94 |
| 8920 | non-targ | B2032 | Co Q9 | ARA_LEAF | p-Super | LC | 36 | 118 |
| 112193 | non-targ | B2043 | Co Q10 | ARA_LEAF | p-Super | LC | 128 | 257 |
| 39120 | non-targ | B2281 | Co Q9 | ARA_SEED_2 | p-USP | LC | 19 | 33 |
| 99620 | non-targ | B2285 | nicotinic acid | ARA_SEED_2 | p-USP | GC | 26 | 42 |
| 138377 | plastidic | B2574 | nicotinic acid | ARA_SEED_2 | p-USP | GC | 24 | 430 |
| 80906 | non-targ | B2739 | Co Q9 | ARA_LEAF | p-Super | LC | 37 | 114 |
| 81075 | non-targ | B2747 | Co Q9 | ARA_LEAF | p-Super | LC | 70 | 125 |
| 81451 | non-targ | B2762 | lipoic acid | ARA_LEAF | p-Super | LC | 472 | 18201 |
| 81980 | non-targ | B2849 | Co Q9 | ARA_LEAF | p-Super | LC | 38 | 73 |
| 42502 | non-targ | B3262 | Co Q9 | ARA_LEAF | p-Super | LC | 49 | 107 |
| 138770 | non-targ | B3749 | Co Q10 | ARA_LEAF | p-Super | LC | 74 | 169 |
| 10708 | non-targ | B3817 | Co Q9 | ARA_LEAF | p-Super | LC | 84 | 98 |
| 44223 | non-targ | B3945 | Co Q9 | ARA_SEED_2 | p-USP | LC | 22 | 37 |
| 138857 | plastidic | B3958 | Co Q10 | ARA_SEED_2 | p-USP | LC | 24 | 32 |
| 10740 | non-targ | B4029 | Co Q9 | ARA_LEAF | p-Super | LC | 41 | 171 |
| 45321 | non-targ | B4256 | Co Q9 | ARA_LEAF | p-Super | LC | 43 | 135 |
| 45394 | non-targ | B4321 | Co Q9 | ARA_LEAF | p-Super | LC | 47 | 110 |
| 46751 | non-targ | GM02LC19289 | Co Q10 | ARA_LEAF | p-PcUBI | LC | 61 | 107 |
| 46751 | non-targ | GM02LC19289 | Co Q9 | ARA_LEAF | p-PcUBI | LC | 53 | 82 |
| 84198 | non-targ | GM02LC21368 | Co Q10 | ARA_LEAF | p-PcUBI | LC | 56 | 519 |
| 49143 | plastidic | Sll0248 | Co Q10 | ARA_LEAF | p-PcUBI | LC | 95 | 573 |
| 84869 | plastidic | Sll0378 | Co Q10 | ARA_LEAF | p-PcUBI | LC | 58 | 77 |
| 55379 | non-targ | Sll1761 | Co Q10 | ARA_LEAF | p-PcUBI | LC | 81 | 152 |
| 55379 | non-targ | Sll1761 | Co Q9 | ARA_LEAF | p-PcUBI | LC | 48 | 91 |
| 55385 | mitoch | Sll1920 | Co Q9 | ARA_LEAF | p-PcUBI | LC | 37 | 55 |
| 11990 | non-targ | Slr0338 | Co Q9 | ARA_LEAF | p-PcUBI | LC | 47 | 62 |
| 87397 | plastidic | Slr0721 | Co Q10 | ARA_LEAF | p-PcUBI | LC | 61 | 135 |
| 87397 | plastidic | Slr0721 | Co Q9 | ARA_LEAF | p-PcUBI | LC | 56 | 88 |
| 119222 | mitoch | Slr0949 | Co Q9 | ARA_LEAF | p-PcUBI | LC | 46 | 50 |
| 101776 | plastidic | Slr1107 | Co Q9 | ARA_LEAF | p-PcUBI | LC | 39 | 135 |
| 116713 | non-targ | Slr1350 | Co Q9 | ARA_LEAF | p-PcUBI | LC | 43 | 67 |
| 58731 | plastidic | Slr1739 | Co Q9 | ARA_LEAF | p-PcUBI | LC | 58 | 64 |
| 58751 | mitoch | Slr1742 | Co Q10 | ARA_LEAF | p-PcUBI | LC | 58 | 125 |
| 58823 | plastidic | Slr1755 | Co Q10 | ARA_LEAF | p-PcUBI | LC | 53 | 133 |
| 58823 | plastidic | Slr1755 | Co Q9 | ARA_LEAF | p-PcUBI | LC | 49 | 132 |
| 12698 | non-targ | TTC0019 | Co Q9 | ARA_LEAF | p-PcUBI | LC | 36 | 141 |
| 61070 | non-targ | TTC0337 | Co Q10 | ARA_LEAF | p-PcUBI | LC | 64 | 181 |
| 61723 | non-targ | TTC1193 | Co Q9 | ARA_LEAF | p-PcUBI | LC | 45 | 91 |
| 62079 | non-targ | TTC1386 | Co Q9 | ARA_LEAF | p-PcUBI | LC | 37 | 147 |
| 62717 | non-targ | Ybr160w | Co Q9 | ARA_LEAF | Big35S | LC | 70 | 78 |
| 96980 | plastidic | Ycl027w | Co Q10 | ARA_LEAF | p-Super | LC | 96 | 120 |
| 14275 | plastidic | Ydl159w | Co Q9 | ARA_LEAF | p-Super | LC | 41 | 74 |
| 130903 | non-targ | Ykr072c | Co Q9 | ARA_LEAF | Big35S | LC | 38 | 91 |
| 102061 | non-targ | Ykr089c | nicotinic acid | ARA_SEED_2 | p-PcUBI | GC | 45 | 52 |
| 139173 | non-targ | Ynl007c | Co Q10 | ARA_LEAF | Big35S | LC | 51 | 78 |
| 139173 | non-targ | Ynl007c | Co Q9 | ARA_LEAF | Big35S | LC | 51 | 98 |
| 92540 | plastidic | Yor244w | Co Q9 | ARA_LEAF | p-Super | LC | 47 | 49 |
| 140110 | non-targ | Ypr054w | Co Q10 | ARA_LEAF | Big35S | LC | 53 | 250 |
| 68132 | non-targ | ZM06LC1143 | Co Q10 | ARA_LEAF | p-PcUBI | LC | 81 | 678 |

Column 1 shows the SEQ ID NO, Column 2 shows the expression type (targeted or non-targeted), Column 3 shows the “gene name” (locus), Column 4 shows the metabolite analyzed, Column 5 indicates the A. thaliana source tissue analyzed, Column 6 indicates the used promoter for expression, Column 7 indicates the analytical method. Columns 8 and 9 show the minimum and the maximum increase of the analyzed metabolite (in percent) in comparison to the wild type (ratio_by _WT, given as percent increase).

The term “non-targ” in Table d, Column 2 which shows the expression type means “non-targeted”.

The term “mitoch” in Table d, Column 2 which shows the expression type means “mitochondrial”.

The term “Co Q9 ” in Table d, Column 4 which shows the metabolite means “coenzyme Q9”.

The term “Co Q10 ” in Table d, Column 4 which shows the metabolite means “coenzyme Q10”.

When the analyses were repeated independently, all results proved to be significant.

[0505.1.7.16] to [0515.1.7.16] for the disclosure of these paragraphs see [0505.1.7.7] to [0515.1.7.7] above

In a further embodiment, the present invention relates in paragraphs [0000.1.7.17.] to [0514.1.7.17.] to a further process for the production of the fine chemical 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, and/or verbascose as defined below and corresponding embodiments as described herein as follows.

for the disclosure of this paragraph see [0001.1.7.7] above

Carbohydrates are aldehyde or ketone compounds with multiple hydroxyl groups. Many carbohydrates have the empirical formula (CH2O)n; some also contain nitrogen, phosphorus, or sulfur.

Carbohydrates are classsfied in monosaccharides, oligosaccharides, and polysaccharides.

Monosaccharides, or simple sugars, consist of a single polyhydroxy aldehyde or ketone unit.

Monosaccharides of more than four carbons tend to have cyclic structures.

Oligosaccharides consist of short chains of monosaccharide units, or residues, usually 2 to 19 units, joined by glycosidic bonds.

The polysaccharides are sugar polymers containing more than 20 or so monosaccharide units, and some have hundreds or thousands of units. Some polysaccharides are linear chains; others are branched.

Carbohydrates are called saccharides or, if they are relatively small, sugars.

The most carbohydrates found in nature occur as polysaccharides which are polymers of medium to high molecular weight. Polysaccharides, also called glycans, differ from each other in the identity of their recurring monosaccharide units, in the length of their chains, in the types of bonds linking the units, and in the degree of branching.

In the present invention, saccharides means all of the aforementioned carbohydrate, e.g. monosaccharides, preferably fructose and/or glucose, or other pentoses or hexoses; in free or bound form, preferably glucose-1-phosphate and/or sedoheptulose-7-phosphate; and/or derivates of monosaccharides, preferably (sugar) acids, e.g. saccharic acid, 3,4,5-trihydroxypentanoic acid; and/or sugar alcohols e.g. erythrol, galactitol, sorbitol, mannitol and/or myo-inositol; and/or deoxy-derivates, e.g. fucose; and/or disaccharides, preferably sucrose, melibiose, galactinol and/or trehalose, and/or oligosaccharides, preferably the RFOs (raffinose family of oligosaccharides: preferably the trisaccharide raffinose and/or the pentasaccharide verbascose), and/or polysaccharides, preferably starch and/or cellulose.

Carbohydrates are the most abundant class of organic compounds found in living organisms.

They are a major source of metabolic energy, both for plants and for animals. Aside from the sugars and starches that meet this vital nutritional role, carbohydrates function in energy storage (for example starch or glycogen), in signaling (by glycoproteins and glycolipids, e.g. blood group determinants), fuel the nervous system, muscle and virtually all cells, are parts of nucleic acids (in genes, mRNA, tRNA, ribosomes), and as cell surface markers as recognition sites on cell surfaces and signaling in glycolipids and glycoproteins and also serve as a structural material for example as cell wall components (cellulose).

Fructose commonly known as fruit sugar, is a simple keto-hexose widely distributed in organism, plants, and animals and tasted sweeter than any other carbohydrate. Fructose is used a sweetener by diabetics because it does not rise the blood sugar level and therefore often recommended for, and consumed by, people with diabetes mellitus or hypoglycemia. The most important metabolic derivates are fructose 1,6-biphosphate and 1-phosphate.

Fructose is quicker to absorb moisture and slower to release it to the environment than sucrose, dextrose, or other nutritive sweeteners. Fructose is an excellent humectant and retains moisture for a long period of time even at low relative humidity (RH). Therefore, fructose can contribute to improved quality, better texture, and longer shelf life to the food products in which it is used.

Fructose has a greater effect on freezing point depression than disaccharides or oligosaccharides, which may protect the integrity of cell walls of fruit by reducing ice crystal formation. Fructose is usually produced from starch by enzymatically transforming it into glucose syrup and subsequently treating with an isomerase, leading to a conversion of glucose to fructose.

Glucose, a hexose, also called dextrose, is the most widely distributed sugar in the plant and animal kingdoms and it is the sugar present in blood as “blood sugar”. It occupies a central position in the metabolism of plants, animals, and many microorganisms. Glucose is rich in potential energy, and thus a good fuel; in the body it is catabolised to produce ATP. It is stored as a high molecular weight polymer such as starch or glycogen or is converted to fatty acids.

Use of glucose as an energy source in cells is via aerobic or anaerobic respiration. Both of these start with the early steps of the glycolysis metabolic pathway.lt is also a remarkably versatile precursor, capable of supplying a huge array of metabolic intermediates for biosynthetic reactions. Specially the phosphoric acid esters of glucose are extremely metabolic intermediastes. Based on its manifold features, glucose is used in nutrition and medicine.

Fructose and glucose are the main carbohydrate constituents of honey. Those hexoses are further the main components of many oligo- and polysaccharides, like sucrose, raffinose, stachyose, trehalose, starch, cellulose or dextran.

Glucose 1-phosphate (also called cori ester) is a glucose molecule with a phosphate group on the 1′-carbon. In glycogenolysis, it is the direct product of the reaction in which glycogen phosphorylase cleaves off a molecule of glucose from a greater glycogen structure. In cellular catabolism it must first be converted to glucose 6-phosphate by the enzyme phosphoglucomutase. In glycogenesis, free glucose 1-phosphate can also react with UTP to form UDP-glucose, by using the enzyme UDP-glucose pyrophosphorylase. Uridine diphosphate glucose (UDP-glucose) is further of widespread general importance in carbohydrate metabolism, especially in glycogen and murein synthesis.

Sedoheptulose-7-phosphate ([(2R,4R,5S)-2,3,4,5,7-pentahydroxy-6-oxoheptyl] phosphate, sedoheptulose-7-P, CAS No: 2646-35-7) is an intermediate of the pentose phosphate pathway and in plants an intermediate of the non oxidative pentose phosphate pathway and the calvin cycle. Likewise, Seduheptulose-7-phosphate is synthesised via the nonoxidativ pentose phosphate pathway in bacteria.

It is formed by transketolase and acted upon by transaldolase.

Sedoheptulose-7-phosphate is a precursor in the synthesis of lipopolysaccharides in bacteria. Heptose-deficient bacterial mutants are more sensitive to antibiotics and bile.

Saccharic acid (Glucaric acid, glucarate, DL-Glucaric acid, Glucosaccharic acid,Tetrahydroxyadipic acid, CAS No.: 87-73-0) is a natural, apparently non-toxic compound produced in small amounts by mammals, including humans and by some plants.

Glucaric acid or its derivatives have been found in the latex of a succulent plant, mung bean seedlings, seedlings and needles of gymnosperms, latex, leaves, or stems of different succulent plants and tomato leaves. Glucaric acid has been detected in sweet cherry fruits and citrus fruits.

For industrial application saccharic acid is derived by oxidizing a sugar such as glucose with nitric acid. The synthesis of saccharic acid via the oxidation of glucose with nitric acid is very cost intensive and limits its use. Hence, an alternative process for the synthesis of saccharic acid with high yielding concentration of saccharic acid at low costs would support the use of this acid. Saccharic acid can be used for example as detergent to substitute for phosphates, as corrosion inhibitor and as a building block in the synthesis of polymers, such as nylon. Furthermore, glucaric acid is being explored as cancer preventive agent (Walaszek Z; Cancer Lett. 1990 Oct. 8; 54 (1-2):1-8).

3,4,5-Trihydroxypentanoic acid is sugar acid derived from xylonic acid as 2-deoxy-xylonic acid. The 3,4,5-trihydroxypentanoic acid gamma-lactone (3,4,5-TP) decrease food intake and depresse the size of the first meal after infusion and therefore seems to be involved in feeding modulation by conveying intrinsically reciprocal signals to neurons involved in feeding and satiety (Toshiie Sakata, Brain Res., vol. 473, p. 43, 1988).

In the majority of plants, the main carbohydrate transported is saccharose. However in a large number of plants, other compounds, such as polyols, are also transported. Polyols (or sugar alcohols) are, like saccharose, primary products of photosynthesis and include mannitol, sorbitol, dulcitol, galactitol, inositol, myo-inositol, ribitol, xylitol and more. Moreover, polyols are although discussed as plant osmoprotectants. They have also been postulated as scavengers of activated oxygen species preventing peroxidation of lipids and resulting cell damage.

Erythrol (Phycitol, Erythritol, Erythrite, Phycite, meso-1,2,3,4-Tetrahydroxybutan, ((2R,3S)butane-1,2,3,4-tetraol), E 968, CAS NO 149-32-6) belongs, together with the more abundantly encountered sorbitol and mannitol, to the group of sugar alcohols or polyols. At least in some plant species, polyols are the preffered long distance transport form of photoassimilates, but they are although discussed as plant osmoprotectants. They have also been postulated as scavengers of activated oxygen species preventing peroxidation of lipids and resulting cell damage. Erythrol occurs naturally in fruits such as pears, melons and grapes, as well as foods such as mushrooms and fermentation-derived foods such as wine, soy sauce and cheese.

Since 1990, erythritol has been commercially produced and added to foods and beverages to provide sweetness, as well as enhance their taste and texture.

Erythritol has a strong cooling effect when it dissolves in water, often combined with the cooling effect of mint flavors, but proves distracting with more subtle flavors and textures. The cooling effect of erythritol is very similar to that of xylitol and among the strongest cooling effects of all sugar alcohols. Erythritol is commonly used as a medium in which to deliver high intensity sweeteners, especially stevia derivatives, serving the dual function of providing both bulk and additional flavor. Diet beverages made with this blend thus also contain erythritol in addition to the main sweetener. Erythritol has been certified as tooth-friendly. The sugar alcohol cannot be metabolized by oral bacteria, and so does not contribute to tooth decay.

Erythritol is produced by microbial methods using mostly osmophilic yeasts and has been produced commercially using mutant strains of Aureobasidium sp. and Pseudozyma tsukubaensis.

Due to the high yield and productivity in the industrial scale of production, erythritol serves as an inexpensive starting material for the production of other sugars (Moon et al., Appl. Micob.y and Biotech., Vol 86(4), 1017-1025).

Galactitol (Dulcitol, (2R,3S,4R,5S)-hexane-1,2,3,4,5,6-hexol) is a sugar alcohol (a polyol), the reduction product of galactose. Three of the sugar alcohols are widely distributed in Angiosperms: galactitol, mannitol and sorbitol.

Sorbitol ((2S,3R,4R,5R)-Hexane-1,2,3,4,5,6-hexol, E 420, D-glucitol, CAS-No 50-70-4) is a sugar alcohol that the human body metabolises slowly. It can be obtained by reduction of glucose, changing the aldehyde group to a hydroxyl group.

In the majority of plants, the main glucide transported is saccharose. However in a large number of plants, other compounds, such as polyols, are also transported.

In plants of the Rosaceae family, sorbitol is the primary product of photosynthesis and the major translocation form of photosynthate.

Sorbitol is an intermediate product in industial scale synthesis of ascorbic acid (ReichsteinSythese). Sorbitol is a sugar substitute. It may be listed under the inactive ingredients listed for some foods and products. Sorbitol is hygroscopic and therefore used as a humectant in cosmetic or food preparations. Sorbitol often is used in mouthwash and toothpaste. Some transparent gels can be made only with sorbitol, as it has a refractive index sufficiently high for transparent formulations.

Mannitol ((2R,3R,4R,5R)-Hexane-1,2,3,4,5,6-hexol, E 421, CAS No 69-65-8 (D-isomer), mannite, manna sugar) is the most widely distributed sugar alcohol in nature and has been reported in more than 100 species of vascular plants. In plants, mannitol comprises a significant portion of the soluble carbohydrates in many species. Mannitol-producing plants exhibit a high degree of salt tolerance.

Production of mannitol by extraction of plant raw material (e.g. manna, seaweed, or algae) is no longer economically relevant. Today, mannitol is produced commercially by catalytic hydrogenation of fructose-containing syrups. The existing chemical production methods are, however, characterized by several drawbacks. The uppermost being that when fructose is catalytically hydrogenated only about 50% of it is converted into mannitol, whereas the rest is converted into another sugar alcohol, sorbitol. In addition, ultra-pure (expensive) raw materials (fructose and hydrogen gas) are required for efficient conversion. When more cost-effective raw materials, such as glucose-fructose syrups are used as starting material for catalytic hydrogenation, the main product is sorbitol and mannitol is formed as a by-product. (von Weymarn, N., dissertation, Helskini, 2002).

Mannitol is used clinically to reduce acutely raised intracranial pressure until more definitive treatment can be applied, e.g., after head trauma. It is also used to treat patients with oliguric renal failure. Mannitol can also be used to open the blood-brain barrier by temporarily shrinking the tightly coupled endothelial cells that make up the barrier. This makes mannitol indispensable for delivering various drugs directly to the brain (e.g., in the treatment of Alzheimer's disease).

Mannitol is also the first drug of choice for the treatment of acute glaucoma in veterinary medicine. Mannitol is also used as a sweetener for people with diabetes, and in chewing gums.

An important key intermediates in the formation of RFOs is myo-inositol (cyclohexan1,2,3,4,5,6-hexaole), the most common cyclitol. Myo-inositol is fundamental to many different aspects of plant growth and development. In addition to its role as the precursor for phytic acid biosynthesis, myo-inositol is also used for uronide and pentose biosynthesis, it is also present in phosphoinositides of plant cell membranes, as well as other complex plant lipids including glycophosphoceramides. Furthermore, it is also a precursor of other naturally occurring inositol isomers, and many of these as well as myo-inositol are distributed as methyl ethers in a species specific pattern throughout the plant kingdom. Myo-inositol is an important growth factor.

Myo-inositol exists in nature either in its free form (found, for example, in sugarcane, beet molasses, and almond hulls) or as a hexaphosphate called phytin (found, for example, in corn steep liquor). Industrial purification of phytin from corn steep liquor involves precipitation with calcium, followed by hydrolysis with a strong acid. Separation of free form inositols from plant extracts involves treatment with acid and separation of myo-inositol by column (U.S. Pat. No. 5,482,631) or the use of ion-exchange (U.S. Pat. No. 4,482,761).

Myo-inositol, the major nutritionally active form of inositol, is vital to many biological processes of the body, participating in a diverse range of activities. Myo-inositol is one of nine distinct isomers of inositol. It is for example essential for the growth of rodents, but not for most other animals, including humans. Humans can make myo-inositol endogenously, which they do from glucose. Nevertheless myo-inositol influences certain biological activities inside the body. It may affect behavior and may have anti-depressant and anti-anxiety activities. It is synthesized in general from phytin.

Myo-inositol is metabolized to phosphatidylinositol a small but important component of cell membranes. Phosphatidylinositol can be further converted to phosphatidylinositol-4,5bisphosphate, which is a key intermediate in biological signaling. The various forms of inositol (e.g. phosphatidylinositol or inositides such as 1,4,5-inositoltriphosphate=IP3) are active in cell-to-cell communication, including the transmission of nerve impulses. Tissues that are affected include the brain, liver and muscles. Inositol is an indirect source of glucose and glucoronic acid, which is essential to detoxification by the liver.

Fucose ((35,4R,55,65)-6-Methyltetrahydro-2H-pyran-2,3,4,5-tetraol, 6-Deoxy-L-galactose, CAS number 2438-80-4Y) hexose deoxy sugar is found on N-linked glycans on the mammalian, insect and plant cell surface, and is the fundamental sub-unit of the fucoidan polysaccharide.

In the fucose-containing glycan structures, fucosylated glycans, fucose can exist as a terminal modification or serve as an attachment point for adding other sugars. In human N-linked glycans, fucose is most commonly linked α-1,6 to the reducing terminal beta-N-acetlyglucosamine.

The most frequent disaccharide is sucrose (saccharose, beta-D-fructofuranosyl-alpha-D-glucopyranosid, cane sugar, beet sugar, sugar in a narrow sense of a name for commercially available sucrose meaning sucrose is the sugar that is commonly called “sugar”) which consists of the six-carbon sugars D-glucose and D-fructose. It is formed by plants but not by animals. Sucrose is a major intermediate product of photosynthesis; in many plants it is the principal form in which sugar is transported from the leaves to other parts of the plant body. In mammalians sucrose is an obligatory component of blood and its content in blood is kept at the stable level. It is strongly necessary for brain cells as well as for normal functioning of the central nervous system. Sugar is widely-known as a source of glycogen—a substance, feeding liver, heart and muscles. It is one of the most widely-used food products and is the major disaccharide in most diets. It is present in honey, maple sugar, fruits, berries, and vegetables. It may be added to food products as liquid or crystalline sucrose or as invert sugar. It is commercially prepared from sugar cane or sugar beets. Sucrose can provide a number of desirable functional qualities to food products including sweetness, mouth-feel, and the ability to transform between amorphous and crystalline states. High-concentrated sucrose is a natural preserving agent, it determines gel-formation processes, gives necessary viscosity to the products. Sucrose is a raw material for caramel, colour etc.

Sucrose is further an excellent fermentation feedstock, which is of specific interest for fermentation industry (including a number of non-food industries -pharmaceutical industries). The presence of eight hydroxyl groups in the sucrose molecule provides a theoretical possibility of a very large number of sucrose derivatives. Sucrose derivatives are used by industries in production of detergents, emulsifiers (sucrose +fatty acids) and adhesives (sucrose octa acetate).

Sucrose—commonly referred to as table sugar—is a disaccharide comprising glucose and fructose. Succrose is obtained comercially from the expressed juice of sugar cane or of sugar beet. The refining process removes impurities from the sugar plant, producing white sugar crystals. Melibiose (6-0-(alpha-D-Galactopyranosyl)-D-glucopyranose, 6-(α-D-galactosido)-D-glucose, CAS NO 585-99-9) is a disaccharide formed by an alpha linkage between galactose and glucose (D-Gal-α(1→6)-D-Glc). It can be formed by invertase mediated hydrolysis of raffinose, which produces melibiose and fructose.

In literature, melibiose is primarily discussed as side product in fermentation of raffinose, but metabolic profiling in Arabidopsis has shown that melibiose is actually found in plants (Fiehn et al., Nat Biotechnol. 2000 Nov; 18(11):1157-61).

Melibiose is yielded by fermentation of raffinose with top yeast (Hudson and Harding, J. Am. Chem. Soc., 1915, 37 (12), pp 2734-2736). Galactinol (1-O-alpha-D-Galactopyranosyl-L-myo-inositol, CAS NO 16908-86-4 (galactinol dihydrate) is involved together with raffinose in tolerance to drought, high salinity and cold stress and may function as osmoprotectants in drought-stress tolerance. Stress inducible enzyme play a key role in the accumulation of galactinol and raffinose under abiotic stress conditions (BRENDA-Enzyme Information System).

Galactinol is an essential intermediate in the biosyntheis of raffinose saccharides in plant tissues (Kuo, JAOCS (1992), 69(6) 569-574). Galactinol was found in cold-stored potato tubers, but was reported to be absent in freshly harvested tubers (Pressey and Shaw, Potato research (1969), 12(2), 64-66). Trehalose (tremalose, 1-α-Glucopyranosyl-1-α-Glucopyranosid, CAS Reg. No. 99-20-7 (anhydrous trehalose), CAS Reg. No. 6138-23-4 (trehalose dihydrate) is a disaccharide formed by a 1,1-glucoside bond between two α-glucose units. Because trehalose is formed by the bonding of the reducing groups of glucose, trehalose has no reducing power.

Trehalose can be found in many organisms, including bacteria, fungi, insects, plants, and invertebrates. Trehalose protects organisms against various stresses, such as dryness, freezing, and osmopressure. Anhydrobitic organisms are able to tolerate the lack of water owing to their ability to synthesize large quantities of trehalose, and the trehalose plays a key role in stabilizing membranes and other macromolecular assemblies under extreme environmental conditions. (Pure Appl. Chem. (2001), Vol. 74, No. 7, pp. 1263-1269).

Transgenic crop plants with increased trehalose content (aimed at top provide enhanced stress tolerance) have been described (e.g. Penna, Trends in Plant Science (2003), 8(8), 355-357).

The conventional method for production of Trehalose was extraction from yeast, but suffered from low yields and high costs.

Mushrooms contain up to 10-25% trehalose by dry weight (Pure Appl. Chem., Vol. 74, No. 7, pp. 1263-1269, 2002.).

Trehalose has been accepted as a novel food ingredient under the GRAS terms in the U.S. and the EU. Because of its unique characteristics (e.g. beeing a non-reducing sugar and therefore inert to reactions with proteins or free amino acids, beeing stable at low pH and elevated temperatures etc., low hygroscopicity), trehalose is appraised as highly usefull food indegrent.

Sucrose is a precursor to a group of carbohydrates in plants known as the raffinose family of oligosaccharides (RFO) found in many plant seeds especially legumes, for example in beans, cabbage, brussels sprouts, broccoli, asparagus, other vegetables, and whole grains (Shallenberger et al., J.Agric. Food Chem., 9, 1372; 1961). The Raffinose Family of Oligosaccharides (RFO) are alpha-galactosyl derivatives of sucrose. RFOs are synthesized by a set of galactosyltransferases, which sequentially add galactose units from galactinol to sucrose. The most common RFOs are the trisaccharide raffinose, the tetrasaccharide stachyose, and the pentasaccharide verbascose. The RFO's are almost ubiquitous in the plant kingdom, being found in a large variety of seeds from many different families, and they rank second only to sucrose in abundance as soluble carbohydrates. Raffinose family oligosaccharides (RFOs) fulfil multiple functions in plants. In seeds, they possibly protect cellular structures during desiccation and constitute carbon reserves for early germination.

Raffinose (beta-D-fructofuranosyl-6-O-alpha-D-galactopyranosyl-alpha-D-glucopyranosid, melitriose, gossypose, melitose), which consists of sucrose with a-galactose attached through its C-4 atom to the 1 position on the fructose residue or in other words raffinose is a complex carbohydrate, a trisaccharide composed of galactose, fructose, and glucose. It is thought to be second only to sucrose among the nonstructural carbohydrates with respect to abundance in the plant kingdom. It may be ubiquitous, at least among higher plants. Raffinose accumulate in significant quantities in the edible portion of many economically significant crop species. Examples include soybean, sugar beet, cotton, canola and all of the major edible leguminous crops including beans, peas, lentil and lupine.

Verbascose, the pentasaccharide of the raffinose family of oligosaccharides (RFO), occurs in the phloem and in storage organs of many plant species, particularly in seeds of legumes. The physiological functions of verbascose in seeds have been little studied. As its lower homologues raffinose and stachyose, verbascose may act as a protective agent during maturation drying or as carbon reserve for germination. Considerable variation exists in the content of verbascose in seeds.

Thus, saccharides of the invention, e.g. monosaccharides, preferably fructose and/or glucose, or other pentoses or hexoses; in free or bound form, preferably glucose-1-phosphate and/or sedoheptulose-7-phosphate; and/or derivates of monosaccharides, preferably (sugar) acids, e.g. saccharic acid, 3,4,5-trihydroxypentanoic acid; and/or sugar alcohols e.g. erythrol, galactitol, sorbitol, mannitol and/or myo-inositol; and/or deoxy-derivates, e.g. fucose; and/or disaccharides, preferably sucrose, melibiose, galactinol and/or trehalose, and/or oligosaccharides, preferably the RFOs (raffinose family of oligosaccharides: preferably the trisaccharide raffinose and/or the pentasaccharide verbascose), and/or polysaccharides, preferably starch and/or cellulose can be used in a lot of different applications, for example in cosmetics, pharmaceuticals and in feed and food.

Therefore improving the productivity of said saccharides and improving the quality of cosmetics, pharmaceuticals, foodstuffs and animal feeds, in particular of nutrition supplements, is an important task of the different industries.

The content of some of the saccharides in a cell was also increased by increasing or decreasing certain activities as disclosed in WO2006/069610, WO2007/087815 or WO2008/034648.

Futher economical and less expensive methods for producing saccharides of the invention, e.g. monosaccharides, preferably fructose and/or glucose, or other pentoses or hexoses; in free or bound form, preferably glucose-1-phosphate and/or sedoheptulose-7-phosphate; and/or derivates of monosaccharides, preferably (sugar) acids, e.g. saccharic acid, 3,4,5-trihydroxypentanoic acid; and/or sugar alcohols e.g. erythrol, galactitol, sorbitol, mannitol and/or myo-inositol; and/or deoxy-derivates, e.g. fucose; and/or disaccharides, preferably sucrose, melibiose, galactinol and/or trehalose, and/or oligosaccharides, preferably the RFOs (raffinose family of oligosaccharides: preferably the trisaccharide raffinose and/or the pentasaccharide verbascose), and/or polysaccharides, preferably starch and/or celluloseand food- and feedstuffs with defined, balanced and if necessarily increased saccharid content are therefore very important.

Particularly economical methods are biotechnological methods utilizing ssaccharides-producing organisms which are either natural or optimized by genetic modification.

There is a constant need for providing novel enzyme activities or direct or indirect regulators and thus alternative methods with advantageous properties for producing saccharides in organisms, e.g. in transgenic organisms.

Improving the quality of foodstuffs and animal feeds is an important task of the food-and-feed industry. Especially advantageous for the quality of foodstuffs and animal feeds is as balanced as possible and well defined saccharide profile since a great excess of some saccharides above a specific concentration in the food has no further positive.

To ensure a high productivity of said saccharides, in particular monosaccharides, preferably fructose and/or glucose, or other pentoses or hexoses; in free or bound form, preferably glucose-1-phosphate and/or sedoheptulose-7-phosphate; and/or derivates of monosaccharides, preferably (sugar) acids, e.g. saccharic acid, 3,4,5-trihydroxypentanoic acid; and/or sugar alcohols e.g. erythrol, galactitol, sorbitol, mannitol and/or myo-inositol; and/or deoxy-derivates, e.g. fucose; and/or disaccharides, preferably sucrose, melibiose, galactinol and/or trehalose, and/or oligosaccharides, preferably the RFOs (raffinose family of oligosaccharides: preferably the trisaccharide raffinose and/or the pentasaccharide verbascose), and/or polysaccharides, preferably starch and/or cellulose in plants or microorganism, it is necessary to manipulate the natural biosynthesis of said cofactors in said organisms.

Accordingly, there is still a great demand for new and more suitable genes which encode enzymes or other regulators which participate in the biosynthesis of said saccharides, in particular monosaccharides, preferably fructose and/or glucose, or other pentoses or hexoses; in free or bound form, preferably glucose-1-phosphate and/or sedoheptulose-7-phosphate; and/or derivates of monosaccharides, preferably (sugar) acids, e.g. saccharic acid, 3,4,5-trihydroxypentanoic acid; and/or sugar alcohols e.g. erythrol, galactitol, sorbitol, mannitol and/or myo-inositol; and/or deoxy-derivates, e.g. fucose; and/or disaccharides, preferably sucrose, melibiose, galactinol and/or trehalose, and/or oligosaccharides, preferably the RFOs (raffinose family of oligosaccharides: preferably the trisaccharide raffinose and/or the pentasaccharide verbascose), and/or polysaccharides, preferably starch and/or cellulose and make it possible to produce certain said saccharides specifically on an industrial scale without unwanted by products forming. In the selection of genes for biosynthesis two characteristics above all are particularly important. On the one hand, there is as ever a need for improved processes for obtaining the highest possible contents of said cofactors on the other hand as less as possible by products should be produced in the production process.

It is an object of the present invention to develop an inexpensive process for the synthesis of 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, and/or verbascose.

It is a further object of the present invention to develop an inexpensive process for the synthesis of saccharides, in particular monosaccharides, preferably fructose and/or glucose, or other pentoses or hexoses; in free or bound form, preferably glucose-1-phosphate and/or sedoheptulose-7-phosphate; and/or derivates of monosaccharides, preferably (sugar) acids, e.g. saccharic acid, 3,4,5-trihydroxypentanoic acid; and/or sugar alcohols e.g. erythrol, galactitol, sorbitol, mannitol and/or myo-inositol; and/or deoxy-derivates, e.g. fucose; and/or disaccharides, preferably sucrose, melibiose, galactinol and/or trehalose, and/or oligosaccharides, preferably the RFOs (raffinose family of oligosaccharides: preferably the trisaccharide raffinose and/or the pentasaccharide verbascose), and/or polysaccharides, preferably starch and/or cellulose and to assure that said saccharides are more accessible and facilely to isolate and recover in an industrial scale from the producing organism, preferably from a plant.

for the disclosure of this paragraph see [0013.1.7.7] above

Accordingly, in a first embodiment, the invention relates to a process for the production of at least one fine chemical selected from the group consisting of: 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, and verbascose, or, in other words, of the “fine chemical” or “fine chemical of the invention”.

The terms “fine chemical of the invention”, “fine chemical” or “the fine chemical” are used herein equally and relate in context of the paragraphs or sections [0014.1.17.17] to [0514.1.7.17] essentially to the metabolite or the metabolites indicated in column 7, application no. 17 of Tables I, II and IV in the respective line.

Further, the term “in context of any of the paragraphs [0014.1.17.17] to [0514.1.7.17]” as used herein means that for any of said paragraphs [0014.1.17.17] to [0514.1.7.17] the term “the fine chemical” is understood to follow the definition of paragraphs or sections [0014.1.17.17] and [0015.1.17.17], independently whether it refers to any other paragraph or not and whether the reference recites the term “fine chemical” in an other context.

Thus, in cases where one or more paragraphs or sections are incorporated by reference into any of the present paragraphs [0014.1.17.17] to [0514.1.7.17], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to the definition of paragraph [0014.1.17.17] and [0015.1.17.17].

Accordingly, in one embodiment, the term “fine chemical of the invention”, “fine chemical” or “the fine chemical” as used herein (alone or in combination, like in FCRP) means “3,4,5-trihydroxypentanoic acid” in context of the nucleic acid or polypeptide sequences listed in the respective same line of any one of Tables Ito IV of application no. 17 and indicating in column 7 the metabolite “3,4,5-trihydroxypentanoic acid”.

In one embodiment, the term 3,4,5-trihydroxypentanoic acid or the term “fine chemical” mean in context of the paragraphs or sections [0014.1.17.17] to [0514.1.7.17] at least one chemical compound with an activity of the above mentioned 3,4,5-trihydroxypentanoic acid, respectively.

Accordingly, in one embodiment, the term “fine chemical of the invention”, “fine chemical” or “the fine chemical” as used herein (alone or in combination, like in FCRP) means “erythrol” in context of the nucleic acid or polypeptide sequences listed in the respective same line of any one of Tables I to IV of application no. 17 and indicating in column 7 the metabolite “erythrol”.

In one embodiment, the term erythrol or the term “fine chemical” mean in context of the paragraphs or sections [0014.1.17.17] to [0514.1.7.17] at least one chemical compound with an activity of the above mentioned erythrol, respectively.

Accordingly, in one embodiment, the term “fine chemical of the invention”, “fine chemical” or “the fine chemical” as used herein (alone or in combination, like in FCRP) means “fructose” in context of the nucleic acid or polypeptide sequences listed in the respective same line of any one of Tables I to IV of application no. 17 and indicating in column 7 the metabolite “fructose”.

In one embodiment, the term fructose or the term “fine chemical” mean in context of the paragraphs or sections [0014.1.17.17] to [0514.1.7.17] at least one chemical compound with an activity of the above mentioned fructose, respectively.

Accordingly, in one embodiment, the term “fine chemical of the invention”, “fine chemical” or “the fine chemical” as used herein (alone or in combination, like in FCRP) means “fucose” in context of the nucleic acid or polypeptide sequences listed in the respective same line of any one of Tables I to IV of application no. 17 and indicating in column 7 the metabolite “fucose”.

In one embodiment, the term fucose or the term “fine chemical” mean in context of the paragraphs or sections [0014.1.17.17] to [0514.1.7.17] at least one chemical compound with an activity of the above mentioned fucose, respectively.

Accordingly, in one embodiment, the term “fine chemical of the invention”, “fine chemical” or “the fine chemical” as used herein (alone or in combination, like in FCRP) means “galactinol” in context of the nucleic acid or polypeptide sequences listed in the respective same line of any one of Tables I to IV of application no. 17 and indicating in column 7 the metabolite “galactinol”.

In one embodiment, the term galactinol or the term “fine chemical” mean in context of the paragraphs or sections [0014.1.17.17] to [0514.1.7.17] at least one chemical compound with an activity of the above mentioned galactinol, respectively.

Accordingly, in one embodiment, the term “fine chemical of the invention”, “fine chemical” or “the fine chemical” as used herein (alone or in combination, like in FCRP) means “galactitol” in context of the nucleic acid or polypeptide sequences listed in the respective same line of any one of Tables I to IV of application no. 17 and indicating in column 7 the metabolite “galactitol”.

In one embodiment, the term galactitol or the term “fine chemical” mean in context of the paragraphs or sections [0014.1.17.17] to [0514.1.7.17] at least one chemical compound with an activity of the above mentioned galactitol, respectively.

Accordingly, in one embodiment, the term “fine chemical of the invention”, “fine chemical” or “the fine chemical” as used herein (alone or in combination, like in FCRP) means “glucose” in context of the nucleic acid or polypeptide sequences listed in the respective same line of any one of Tables I to IV of application no. 17 and indicating in column 7 the metabolite “glucose”.

In one embodiment, the term glucose or the term “fine chemical” mean in context of the paragraphs or sections [0014.1.17.17] to [0514.1.7.17] at least one chemical compound with an activity of the above mentioned glucose, respectively.

Accordingly, in one embodiment, the term “fine chemical of the invention”, “fine chemical” or “the fine chemical” as used herein (alone or in combination, like in FCRP) means “glucose-1-phosphate” in context of the nucleic acid or polypeptide sequences listed in the respective same line of any one of Tables Ito IV of application no. 17 and indicating in column 7 the metabolite “glucose-1-phosphate”.

In one embodiment, the term glucose-1-phosphate or the term “fine chemical” mean in context of the paragraphs or sections [0014.1.17.17] to [0514.1.7.17] at least one chemical compound with an activity of the above mentioned glucose-1-phosphate, respectively.

Accordingly, in one embodiment, the term “fine chemical of the invention”, “fine chemical” or “the fine chemical” as used herein (alone or in combination, like in FCRP) means “mannitol” in context of the nucleic acid or polypeptide sequences listed in the respective same line of any one of Tables I to IV of application no. 17 and indicating in column 7 the metabolite “mannitol”.

In one embodiment, the term mannitol or the term “fine chemical” mean in context of the paragraphs or sections [0014.1.17.17] to [0514.1.7.17] at least one chemical compound with an activity of the above mentioned mannitol, respectively.

Accordingly, in one embodiment, the term “fine chemical of the invention”, “fine chemical” or “the fine chemical” as used herein (alone or in combination, like in FCRP) means “melibiose” in context of the nucleic acid or polypeptide sequences listed in the respective same line of any one of Tables I to IV of application no. 17 and indicating in column 7 the metabolite “melibiose”.

In one embodiment, the term melibiose or the term “fine chemical” mean in context of the paragraphs or sections [0014.1.17.17] to [0514.1.7.17] at least one chemical compound with an activity of the above mentioned melibiose, respectively.

Accordingly, in one embodiment, the term “fine chemical of the invention”, “fine chemical” or “the fine chemical” as used herein (alone or in combination, like in FCRP) means “myo-inositol” in context of the nucleic acid or polypeptide sequences listed in the respective same line of any one of Tables I to IV of application no. 17 and indicating in column 7 the metabolite “myo-inositol”.

In one embodiment, the term myo-inositol or the term “fine chemical” mean in context of the paragraphs or sections [0014.1.17.17] to [0514.1.7.17] at least one chemical compound with an activity of the above mentioned myo-inositol, respectively.

Accordingly, in one embodiment, the term “fine chemical of the invention”, “fine chemical” or “the fine chemical” as used herein (alone or in combination, like in FCRP) means “raffinose” in context of the nucleic acid or polypeptide sequences listed in the respective same line of any one of Tables I to IV of application no. 17 and indicating in column 7 the metabolite “raffinose”.

In one embodiment, the term raffinose or the term “fine chemical” mean in context of the paragraphs or sections [0014.1.17.17] to [0514.1.7.17] at least one chemical compound with an activity of the above mentioned raffinose, respectively.

Accordingly, in one embodiment, the term “fine chemical of the invention”, “fine chemical” or “the fine chemical” as used herein (alone or in combination, like in FCRP) means “saccharic acid” in context of the nucleic acid or polypeptide sequences listed in the respective same line of any one of Tables Ito IV of application no. 17 and indicating in column 7 the metabolite “saccharic acid”.

In one embodiment, the term saccharic acid or the term “fine chemical” mean in context of the paragraphs or sections [0014.1.17.17] to [0514.1.7.17] at least one chemical compound with an activity of the above mentioned saccharic acid, respectively.

Accordingly, in one embodiment, the term “fine chemical of the invention”, “fine chemical” or “the fine chemical” as used herein (alone or in combination, like in FCRP) means “sedoheptulose-7-phosphate” in context of the nucleic acid or polypeptide sequences listed in the respective same line of any one of Tables Ito IV of application no. 17 and indicating in column 7 the metabolite “sedoheptulose-7-phosphate”.

In one embodiment, the term sedoheptulose-7-phosphate or the term “fine chemical” mean in context of the paragraphs or sections [0014.1.17.17] to [0514.1.7.17] at least one chemical compound with an activity of the above mentioned sedoheptulose-7-phosphate, respectively.

Accordingly, in one embodiment, the term “fine chemical of the invention”, “fine chemical” or “the fine chemical” as used herein (alone or in combination, like in FCRP) means “sorbitol” in context of the nucleic acid or polypeptide sequences listed in the respective same line of any one of Tables I to IV of application no. 17 and indicating in column 7 the metabolite “sorbitol”.

In one embodiment, the term sorbitol or the term “fine chemical” mean in context of the paragraphs or sections [0014.1.17.17] to [0514.1.7.17] at least one chemical compound with an activity of the above mentioned sorbitol, respectively.

Accordingly, in one embodiment, the term “fine chemical of the invention”, “fine chemical” or “the fine chemical” as used herein (alone or in combination, like in FCRP) means “sucrose” in context of the nucleic acid or polypeptide sequences listed in the respective same line of any one of Tables I to IV of application no. 17 and indicating in column 7 the metabolite “sucrose”.

In one embodiment, the term sucrose or the term “fine chemical” mean in context of the paragraphs or sections [0014.1.17.17] to [0514.1.7.17] at least one chemical compound with an activity of the above mentioned sucrose, respectively.

Accordingly, in one embodiment, the term “fine chemical of the invention”, “fine chemical” or “the fine chemical” as used herein (alone or in combination, like in FCRP) means “trehalose” in context of the nucleic acid or polypeptide sequences listed in the respective same line of any one of Tables I to IV of application no. 17 and indicating in column 7 the metabolite “trehalose”.

In one embodiment, the term trehalose or the term “fine chemical” mean in context of the paragraphs or sections [0014.1.17.17] to [0514.1.7.17] at least one chemical compound with an activity of the above mentioned trehalose, respectively.

Accordingly, in one embodiment, the term “fine chemical of the invention”, “fine chemical” or “the fine chemical” as used herein (alone or in combination, like in FCRP) means “verbascose” in context of the nucleic acid or polypeptide sequences listed in the respective same line of any one of Tables I to IV of application no. 17 and indicating in column 7 the metabolite “verbascose”.

In one embodiment, the term verbascose or the term “fine chemical” mean in context of the paragraphs or sections [0014.1.17.17] to [0514.1.7.17] at least one chemical compound with an activity of the above mentioned verbascose, respectively.

Accordingly, in one embodiment, the terms “fine chemical of the invention”, “fine chemical” or “the fine chemical” mean in context of any of the paragraphs [0014.1.17.17] to [0514.1.7.17] 3,4,5-trihydroxypentanoic acid, its lacton, salts, ester or ether in free form or bound form. In a preferred embodiment, the term “the fine chemical” means 3,4,5-trihydroxypentanoic acid or its salts, ester or ether, in free form or bound form.

In a preferred embodiment “3,4,5-trihydroxypentanoic acid ” means 3,4,5-trihydroxypentanoic acid in free form.

Accordingly, in one embodiment, the terms “fine chemical of the invention”, “fine chemical” or “the fine chemical” mean in context of any of the paragraphs [0014.1.17.17] to [0514.1.7.17] erythrol, its salts, ester or ether in free form or bound form. In a preferred embodiment, the term “the fine chemical” means erythrol or its salts, ester or ether, in free form or bound form.

In a preferred embodiment “erythrol” means erythrol in free form.

Accordingly, in one embodiment, the terms “fine chemical of the invention”, “fine chemical” or “the fine chemical” mean in context of any of the paragraphs [0014.1.17.17] to [0514.1.7.17] fructose, its salts, ester or ether in free form or bound form. In a preferred embodiment, the term “the fine chemical” means fructose or its salts, ester or ether, in free form or bound form.

Further, the term “the fine chemicals” as used herein also relates in one embodiment to fine chemicals comprising fructose, preferably fructan, amins, ethers, esters, preferably ester of phosphoric acid, e.g. phosphorylated fructose, glycosides, preferably glycolipids and glycoproteins containing fructose.ln a preferred embodiment “fructose” means fructose in free form.

Accordingly, in one embodiment, the terms “fine chemical of the invention”, “fine chemical” or “the fine chemical” mean in context of any of the paragraphs [0014.1.17.17] to [0514.1.7.17] fucose, its salts, ester or ether in free form or bound form. In a preferred embodiment, the term “the fine chemical” means fucose or its salts, ester or ether or fucose-containing glycan structures, in free form or bound form.

In a preferred embodiment “fucose” means fucose in free form.

Accordingly, in one embodiment, the terms “fine chemical of the invention”, “fine chemical” or “the fine chemical” mean in context of any of the paragraphs [0014.1.17.17] to [0514.1.7.17] galactinol, its salts, ester or ether in free form or bound form. In a preferred embodiment, the term “the fine chemical” means galactinol or its salts, ester or ether, in free form or bound form.

In a preferred embodiment “galactinol” means galactinol in free form.

Accordingly, in one embodiment, the terms “fine chemical of the invention”, “fine chemical” or “the fine chemical” mean in context of any of the paragraphs [0014.1.17.17] to [0514.1.7.17] galactitol, its salts, ester or ether in free form or bound form. In a preferred embodiment, the term “the fine chemical” means galactitol or its salts, ester or ether, in free form or bound form.

In a preferred embodiment “galactitol” means galactitol in free form.

Accordingly, in one embodiment, the terms “fine chemical of the invention”, “fine chemical” or “the fine chemical” mean in context of any of the paragraphs [0014.1.17.17] to [0514.1.7.17] glucose, its salts, ester or ether in free form or bound form. In a preferred embodiment, the term “the fine chemical” means glucose or its salts, ester or ether, in free form or bound form.

In a preferred embodiment “glucose” means glucose in free form.

Further, the term “the fine chemicals” as used herein also relates in one embodiment to fine chemicals comprising glucose, preferably ester of phosphoric acid, e.g. phosphorylated glucose like glucose-1-phosphate or glucose-6-phosphate, glycosides, preferably glycolipids and glycoproteins containing glucose.

Accordingly, in one embodiment, the terms “fine chemical of the invention”, “fine chemical” or “the fine chemical” mean in context of any of the paragraphs [0014.1.17.17] to [0514.1.7.17] glucose-1-phosphate, its salts, ester or ether in free form or bound form. In a preferred embodiment, the term “the fine chemical” means glucose-1-phosphate or its salts, ester or ether, in free form or bound form.

In a preferred embodiment “glucose-1-phosphate” means glucose-1-phosphate in free form.

Accordingly, in one embodiment, the terms “fine chemical of the invention”, “fine chemical” or “the fine chemical” mean in context of any of the paragraphs [0014.1.17.17] to [0514.1.7.17] mannitol, its salts, ester or ether in free form or bound form. In a preferred embodiment, the term “the fine chemical” means mannitol or its salts, ester or ether, in free form or bound form.

In a preferred embodiment “mannitol” means mannitol in free form.

Accordingly, in one embodiment, the terms “fine chemical of the invention”, “fine chemical” or “the fine chemical” mean in context of any of the paragraphs [0014.1.17.17] to [0514.1.7.17] melibiose, its salts, ester or ether in free form or bound form. In a preferred embodiment, the term “the fine chemical” means melibiose or its salts, ester or ether, in free form or bound form.

In a preferred embodiment “melibiose” means melibiose in free form.

Accordingly, in one embodiment, the terms “fine chemical of the invention”, “fine chemical” or “the fine chemical” mean in context of any of the paragraphs [0014.1.17.17] to [0514.1.7.17] myo-inositol, its salts, ester or ether in free form or bound form. In a preferred embodiment, the term “the fine chemical” means myo-inositol or its salts, ester or ether, in free form or bound form. Further, the term “the fine chemicals” as used herein also relates in one embodiment to fine chemicals comprising myo-inositol, preferably ester of phosphoric acid, e.g. phosphorylated myo-inositol like 1-phosphatidyl-inosit or my-inositol-1,2,3,4,5,6-hexakisphosphate (phytate), glycosides, preferably glycolipids like phospholipids and glycoproteinslikephosphoproteins containing my-inositol.

In a preferred embodiment “myo-inositol” means myo-inositol in free form.

Accordingly, in one embodiment, the terms “fine chemical of the invention”, “fine chemical” or “the fine chemical” mean in context of any of the paragraphs [0014.1.17.17] to [0514.1.7.17] raffinose, its salts, ester or ether in free form or bound form. In a preferred embodiment, the term “the fine chemical” means raffinose or its salts, ester or ether, in free form or bound form.

In a preferred embodiment “raffinose” means raffinose in free form.

Accordingly, in one embodiment, the terms “fine chemical of the invention”, “fine chemical” or “the fine chemical” mean in context of any of the paragraphs [0014.1.17.17] to [0514.1.7.17] saccharic acid, its salts, ester or ether in free form or bound form. In a preferred embodiment, the term “the fine chemical” means saccharic acid or its salts, ester or ether, in free form or bound form.

In a preferred embodiment “saccharic acid ” means saccharic acid in free form.

Accordingly, in one embodiment, the terms “fine chemical of the invention”, “fine chemical” or “the fine chemical” mean in context of any of the paragraphs [0014.1.17.17] to [0514.1.7.17] sedoheptulose-7-phosphate, its salts, ester or ether in free form or bound form. In a preferred embodiment, the term “the fine chemical” means sedoheptulose-7-phosphate or its salts, ester or ether, in free form or bound form.

In a preferred embodiment “sedoheptulose-7-phosphate” means sedoheptulose-7-phosphate in free form.

Accordingly, in one embodiment, the terms “fine chemical of the invention”, “fine chemical” or “the fine chemical” mean in context of any of the paragraphs [0014.1.17.17] to [0514.1.7.17] sorbitol, its salts, ester or ether in free form or bound form. In a preferred embodiment, the term “the fine chemical” means sorbitol or its salts, ester or ether, in free form or bound form.

In a preferred embodiment “sorbitol” means sorbitol in free form.

Accordingly, in one embodiment, the terms “fine chemical of the invention”, “fine chemical” or “the fine chemical” mean in context of any of the paragraphs [0014.1.17.17] to [0514.1.7.17] sucrose, its salts, ester or ether in free form or bound form. In a preferred embodiment, the term “the fine chemical” means sucrose or its salts, ester or ether, in free form or bound form.

In a preferred embodiment “sucrose” means sucrose in free form.

Accordingly, in one embodiment, the terms “fine chemical of the invention”, “fine chemical” or “the fine chemical” mean in context of any of the paragraphs [0014.1.17.17] to [0514.1.7.17] trehalose, its salts, ester or ether in free form or bound form. In a preferred embodiment, the term “the fine chemical” means trehalose or its salts, ester or ether, in free form or bound form.

In a preferred embodiment “trehalose” means trehalose in free form.

Accordingly, in one embodiment, the terms “fine chemical of the invention”, “fine chemical” or “the fine chemical” mean in context of any of the paragraphs [0014.1.17.17] to [0514.1.7.17] verbascose, its salts, ester or ether in free form or bound form. In a preferred embodiment, the term “the fine chemical” means verbascose or its salts, ester or ether, in free form or bound form.

In a preferred embodiment “verbascose” means verbascose in free form. Accordingly, in the present invention, the term “the fine chemical” as used herein relates in one embodiment to “saccharides, meaning carbohydrates, e.g. monosaccharides, preferably fructose and/or glucose, or other pentoses or hexoses; in free or bound form, preferably glucose-1-phosphate and/or sedoheptulose-7-phosphate; and/or derivates of monosaccharides, preferably (sugar) acids, e.g. saccharic acid, 3,4,5-trihydroxypentanoic acid; and/or sugar alcohols e.g. erythrol, galactitol, sorbitol, mannitol and/or myo-inositol; and/or deoxy-derivates, e.g. fucose; and/or disaccharides, preferably sucrose, melibiose, galactinol and/or trehalose, and/or oligosaccharides, preferably the RFOs (raffinose family of oligosaccharides: preferably the trisaccharide raffinose and/or the pentasaccharide verbascose), and/or polysaccharides, preferably starch and/or cellulose”. Further, the term “the fine chemicals” as used herein also relates in one embodiment to fine chemicals comprising saccharides, meaning carbohydrates, e.g. monosaccharides, preferably fructose and/or glucose, or other pentoses or hexoses; in free or bound form, preferably glucose-1-phosphate and/or sedoheptulose-7-phosphate; and/or derivates of monosaccharides, preferably (sugar) acids, e.g. saccharic acid, 3,4,5-trihydroxypentanoic acid; and/or sugar alcohols e.g. erythrol, galactitol, sorbitol, mannitol and/or myo-inositol; and/or deoxy-derivates, e.g. fucose; and/or disaccharides, preferably sucrose, melibiose, galactinol and/or trehalose, and/or oligosaccharides, preferably the RFOs (raffinose family of oligosaccharides: preferably the trisaccharide raffinose and/or the pentasaccharide verbascose), and/or polysaccharides, preferably starch and/or cellulose, preferably amins, ethers, esters, preferably ester of phosphoric acid, e.g. phosphorylated saccharides, glycosides, preferably glycolipids and glycoproteins containing said saccharides.

Further, the term “in context of any of the paragraphs [0014.1.17.17] to [0514.1.7.17]” as used herein means that for any of said paragraphs [0014.1.17.17] to [0514.1.7.17] the term “the fine chemical” is understood to follow the definition of section [0014.1.17.17] or section [0015.1.17.17], independently whether it refers to any other paragraph or not and whether the reference recites the term “fine chemical” in an other context.

Thus, in cases where one or more paragraphs or sections are incorporated by reference into any of the present paragraphs [0014.1.17.17] to [0514.1.7.17], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to this definition of this paragraph [0015.1.17.17].

Further, the term “fine chemicals” as used herein relates to compositions comprising said fine chemical(s), i.e. comprising 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, and/or verbascose, respectively.

Accordingly, the present invention relates to a process for the production of raffinose, which comprises
(a) increasing or generating one or more activities selected from the group consisting of galactinol synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose-1-phosphate, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of glucose-1-phosphate, which comprises
(a) increasing or generating one or more activities selected from the group consisting of At1g19350-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of At1g19350-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of myo-inositol, which comprises
(a) increasing or generating one or more activities selected from the group consisting of chlorophyllase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating one or more activities selected from the group consisting of cullin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of fructose, which comprises
(a) increasing or generating one or more activities selected from the group consisting of eukaryotic translation initiation factor 5 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of eukaryotic translation initiation factor 5 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of raffinose, which comprises
(a) increasing or generating one or more activities selected from the group consisting of eukaryotic translation initiation factor 5 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sucrose, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of raffinose, which comprises
(a) increasing or generating one or more activities selected from the group consisting of galactinol synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sucrose, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of saccharic acid, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

Accordingly, the present invention relates to a process for the production of fructose, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of saccharic acid, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

Accordingly, the present invention relates to a process for the production of fructose, which comprises
(a) increasing or generating one or more activities selected from the group consisting of CBL-interacting protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of CBL-interacting protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of sedoheptulose-7-phosphate, which comprises
(a) increasing or generating one or more activities selected from the group consisting of CBL-interacting protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of glucose-1-phosphate, which comprises
(a) increasing or generating one or more activities selected from the group consisting of phenylalanine ammonia-lyase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating one or more activities selected from the group consisting of MADS box protein transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of At2g45420-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of glucose-1-phosphate, which comprises
(a) increasing or generating one or more activities selected from the group consisting of phosphatidylinositol 3- and 4-kinase family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of raffinose, which comprises
(a) increasing or generating one or more activities selected from the group consisting of galactinol synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of raffinose, which comprises
(a) increasing or generating one or more activities selected from the group consisting of galactinol synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of fructose, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of myo-inositol, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of fructose, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of myo-inositol, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating one or more activities selected from the group consisting of thioredoxin family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of thioredoxin family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of myo-inositol, which comprises
(a) increasing or generating one or more activities selected from the group consisting of thioredoxin family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of saccharic acid, which comprises
(a) increasing or generating one or more activities selected from the group consisting of DNA binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

Accordingly, the present invention relates to a process for the production of glucose-1-phosphate, which comprises
(a) increasing or generating one or more activities selected from the group consisting of CCAAT-binding transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of raffinose, which comprises
(a) increasing or generating one or more activities selected from the group consisting of CCAAT-binding transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of fructose, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of sedoheptulose-7-phosphate, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of fructose, which comprises
(a) increasing or generating one or more activities selected from the group consisting of eukaryotic translation initiation factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of eukaryotic translation initiation factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of raffinose, which comprises
(a) increasing or generating one or more activities selected from the group consisting of eukaryotic translation initiation factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of myo-inositol, which comprises
(a) increasing or generating one or more activities selected from the group consisting of mitogen-activated protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sedoheptulose-7-phosphate, which comprises
(a) increasing or generating one or more activities selected from the group consisting of auxin response factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of fructose, which comprises
(a) increasing or generating one or more activities selected from the group consisting of monthiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of monthiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of monthiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of monthiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sedoheptulose-7-phosphate, which comprises
(a) increasing or generating one or more activities selected from the group consisting of monthiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of sucrose, which comprises
(a) increasing or generating one or more activities selected from the group consisting of monthiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sedoheptulose-7-phosphate, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of sucrose, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose-1-phosphate, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of fructose, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sedoheptulose-7-phosphate, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of sucrose, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sedoheptulose-7-phosphate, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of sucrose, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sucrose, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of sedoheptulose-7-phosphate, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of sucrose, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of myo-inositol, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sedoheptulose-7-phosphate, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of sucrose, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose-1-phosphate, which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of raffinose, which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sucrose, which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose-1-phosphate, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of raffinose, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of myo-inositol, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sucrose, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of myo-inositol, which comprises
(a) increasing or generating one or more activities selected from the group consisting of gibberellin 20-oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of myo-inositol, which comprises
(a) increasing or generating one or more activities selected from the group consisting of inositol-3-phosphate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating one or more activities selected from the group consisting of inositol-3-phosphate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of fructose, which comprises
(a) increasing or generating one or more activities selected from the group consisting of At5g16650-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of fructose, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of myo-inositol, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sedoheptulose-7-phosphate, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of sucrose, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose-1-phosphate, which comprises
(a) increasing or generating one or more activities selected from the group consisting of At5g43630-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose-1-phosphate, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating one or more activities selected from the group consisting of RNA-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of RNA-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of fructose, which comprises
(a) increasing or generating one or more activities selected from the group consisting of MADS-box transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of raffinose, which comprises
(a) increasing or generating one or more activities selected from the group consisting of MADS-box transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of saccharic acid, which comprises
(a) increasing or generating one or more activities selected from the group consisting of MADS-box transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

Accordingly, the present invention relates to a process for the production of glucose-1-phosphate, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of raffinose, which comprises
(a) increasing or generating one or more activities selected from the group consisting of glycosyl transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sucrose, which comprises
(a) increasing or generating one or more activities selected from the group consisting of sec-independent protein translocase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of beta-hydroxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating one or more activities selected from the group consisting of elongation factor Tu in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of elongation factor Tu in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of fructose, which comprises
(a) increasing or generating one or more activities selected from the group consisting of ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of sucrose, which comprises
(a) increasing or generating one or more activities selected from the group consisting of ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose-1-phosphate, which comprises
(a) increasing or generating one or more activities selected from the group consisting of acyl-CoA synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose, which comprises
(a) increasing or generating one or more activities selected from the group consisting of 2-oxoglutarate dehydrogenase E1 subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of 2-oxoglutarate dehydrogenase E1 subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of 2-oxoglutarate dehydrogenase E1 subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating one or more activities selected from the group consisting of 2-oxoglutarate dehydrogenase E1 subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sedoheptulose-7-phosphate, which comprises
(a) increasing or generating one or more activities selected from the group consisting of 2-oxoglutarate dehydrogenase E1 subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of raffinose, which comprises
(a) increasing or generating one or more activities selected from the group consisting of GDP-mannose dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of fructose, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of ThiF family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating one or more activities selected from the group consisting of CTP synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of CTP synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of sucrose, which comprises
(a) increasing or generating one or more activities selected from the group consisting of CTP synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of raffinose, which comprises
(a) increasing or generating one or more activities selected from the group consisting of protein-L-isoaspartate O-methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of myo-inositol, which comprises
(a) increasing or generating one or more activities selected from the group consisting of recombinase A in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating one or more activities selected from the group consisting of recombinase A in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of myo-inositol, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of raffinose, which comprises
(a) increasing or generating one or more activities selected from the group consisting of enoyl-CoA hydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of fructose, which comprises
(a) increasing or generating one or more activities selected from the group consisting of NADH-quinone oxidoreductase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose-1-phosphate, which comprises
(a) increasing or generating one or more activities selected from the group consisting of NADH-quinone oxidoreductase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of sucrose, which comprises
(a) increasing or generating one or more activities selected from the group consisting of NADH-quinone oxidoreductase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of isopropylmalate isomerase large subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating one or more activities selected from the group consisting of hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of sucrose, which comprises
(a) increasing or generating one or more activities selected from the group consisting of hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating one or more activities selected from the group consisting of glutamate-ammonia-ligase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of glutamate-ammonia-ligase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of sucrose, which comprises
(a) increasing or generating one or more activities selected from the group consisting of glutamate-ammonia-ligase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating one or more activities selected from the group consisting of fumarylacetoacetate hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of raffinose, which comprises
(a) increasing or generating one or more activities selected from the group consisting of glycosyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of myo-inositol, which comprises
(a) increasing or generating one or more activities selected from the group consisting of peptidyl-prolyl cis-trans isomerase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of NAD(P)H dehydrogenase (quinone) in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating one or more activities selected from the group consisting of electron transfer flavoprotein subunit beta in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of electron transfer flavoprotein subunit beta in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of purine nucleoside phosphorylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of purine nucleoside phosphorylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of raffinose, which comprises
(a) increasing or generating one or more activities selected from the group consisting of transcriptional regulator protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose-1-phosphate, which comprises
(a) increasing or generating one or more activities selected from the group consisting of AX653549-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating one or more activities selected from the group consisting of AY087308-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of AY087308-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of glucose-1-phosphate, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose, which comprises
(a) increasing or generating one or more activities selected from the group consisting of threonine synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of sucrose, which comprises
(a) increasing or generating one or more activities selected from the group consisting of threonine synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of b0050-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of raffinose, which comprises
(a) increasing or generating one or more activities selected from the group consisting of L-ribulose-5-phosphate 4-epimerase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sucrose, which comprises
(a) increasing or generating one or more activities selected from the group consisting of glucose dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose-1-phosphate, which comprises
(a) increasing or generating one or more activities selected from the group consisting of serine protease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of serine protease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sucrose, which comprises
(a) increasing or generating one or more activities selected from the group consisting of acyl-CoA dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose-1-phosphate, which comprises
(a) increasing or generating one or more activities selected from the group consisting of b0251-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of fructose, which comprises
(a) increasing or generating one or more activities selected from the group consisting of betaine aldehyde dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of melibiose, which comprises
(a) increasing or generating one or more activities selected from the group consisting of beta-galactosidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of melibiose or a composition comprising melibiose in said non-human organism or in the culture medium surrounding said non-human organism.

Accordingly, the present invention relates to a process for the production of glucose-1-phosphate, which comprises
(a) increasing or generating one or more activities selected from the group consisting of b0362-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating one or more activities selected from the group consisting of penicillin-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of fructose, which comprises
(a) increasing or generating one or more activities selected from the group consisting of ATP-binding component of a transport system in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of ATP-binding component of a transport system in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose, which comprises
(a) increasing or generating one or more activities selected from the group consisting of membrane transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of saccharic acid, which comprises
(a) increasing or generating one or more activities selected from the group consisting of b0488-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

Accordingly, the present invention relates to a process for the production of galactinol, which comprises
(a) increasing or generating one or more activities selected from the group consisting of glycerate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

Accordingly, the present invention relates to a process for the production of fructose, which comprises
(a) increasing or generating one or more activities selected from the group consisting of isochorismate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of isochorismate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of raffinose, which comprises
(a) increasing or generating one or more activities selected from the group consisting of isochorismate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sucrose, which comprises
(a) increasing or generating one or more activities selected from the group consisting of isochorismate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of B0644-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of saccharic acid, which comprises
(a) increasing or generating one or more activities selected from the group consisting of pyrimidine-specific ribonucleoside hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

Accordingly, the present invention relates to a process for the production of erythrol, which comprises
(a) increasing or generating one or more activities selected from the group consisting of asparagine synthetase B in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of erythrol or a composition comprising erythrol in said non-human organism or in the culture medium surrounding said non-human organism.

Accordingly, the present invention relates to a process for the production of myo-inositol, which comprises
(a) increasing or generating one or more activities selected from the group consisting of sensor histidine kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of myo-inositol, which comprises
(a) increasing or generating one or more activities selected from the group consisting of succinyl-CoA synthetase beta chain in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating one or more activities selected from the group consisting of zinc transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of zinc transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of zinc transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of raffinose, which comprises
(a) increasing or generating one or more activities selected from the group consisting of zinc transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose, which comprises
(a) increasing or generating one or more activities selected from the group consisting of b0801-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of sedoheptulose-7-phosphate, which comprises
(a) increasing or generating one or more activities selected from the group consisting of putrescine transport system permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of raffinose, which comprises
(a) increasing or generating one or more activities selected from the group consisting of leucyl/phenylalanyl-tRNA-protein transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose-1-phosphate, which comprises
(a) increasing or generating one or more activities selected from the group consisting of b0970-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of phosphoanhydride phosphorylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating one or more activities selected from the group consisting of b1003-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of b1003-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of b1003-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of sedoheptulose-7-phosphate, which comprises
(a) increasing or generating one or more activities selected from the group consisting of b1003-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of sucrose, which comprises
(a) increasing or generating one or more activities selected from the group consisting of b1003-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of b1046-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating one or more activities selected from the group consisting of multidrug resistance protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of sucrose, which comprises
(a) increasing or generating one or more activities selected from the group consisting of multidrug resistance protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating one or more activities selected from the group consisting of outer membrane receptor protein precursor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose-1-phosphate, which comprises
(a) increasing or generating one or more activities selected from the group consisting of adenylosuccinate lyase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose, which comprises
(a) increasing or generating one or more activities selected from the group consisting of isocitrate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of b1137-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating one or more activities selected from the group consisting of sodium/proton antiporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating one or more activities selected from the group consisting of b1214-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of b1214-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating one or more activities selected from the group consisting of b1219-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of b1219-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of b1219-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of myo-inositol, which comprises
(a) increasing or generating one or more activities selected from the group consisting of b1234-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of galactinol, which comprises
(a) increasing or generating one or more activities selected from the group consisting of cardiolipin synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

Accordingly, the present invention relates to a process for the production of galactitol, which comprises
(a) increasing or generating one or more activities selected from the group consisting of cardiolipin synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactitol or a composition comprising galactitol in said non-human organism or in the culture medium surrounding said non-human organism.

Accordingly, the present invention relates to a process for the production of sorbitol, which comprises
(a) increasing or generating one or more activities selected from the group consisting of cardiolipin synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sorbitol or a composition comprising sorbitol in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of intracellular septation protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating one or more activities selected from the group consisting of membrane protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of membrane protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of raffinose, which comprises
(a) increasing or generating one or more activities selected from the group consisting of membrane protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sucrose, which comprises
(a) increasing or generating one or more activities selected from the group consisting of membrane protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of myo-inositol, which comprises
(a) increasing or generating one or more activities selected from the group consisting of tryptophan biosynthesis protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating one or more activities selected from the group consisting of b1285-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose-1-phosphate, which comprises
(a) increasing or generating one or more activities selected from the group consisting of phage-related repressor protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating one or more activities selected from the group consisting of b1357-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose, which comprises
(a) increasing or generating one or more activities selected from the group consisting of phenylacetic acid degradation protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of phenylacetic acid degradation protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of sedoheptulose-7-phosphate, which comprises
(a) increasing or generating one or more activities selected from the group consisting of b1410-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of myo-inositol, which comprises
(a) increasing or generating one or more activities selected from the group consisting of b1425-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of glucose-1-phosphate, which comprises
(a) increasing or generating one or more activities selected from the group consisting of lipoprotein precursor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of sedoheptulose-7-phosphate, which comprises
(a) increasing or generating one or more activities selected from the group consisting of lipoprotein precursor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of lipoprotein precursor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of b1445-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating one or more activities selected from the group consisting of b1470-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of fructose, which comprises
(a) increasing or generating one or more activities selected from the group consisting of b1522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose-1-phosphate, which comprises
(a) increasing or generating one or more activities selected from the group consisting of b1522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating one or more activities selected from the group consisting of b1556-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of sedoheptulose-7-phosphate, which comprises
(a) increasing or generating one or more activities selected from the group consisting of b1556-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of glucose-1-phosphate, which comprises
(a) increasing or generating one or more activities selected from the group consisting of b1583-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating one or more activities selected from the group consisting of Fe—S subunit of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of Fe—S subunit of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating one or more activities selected from the group consisting of acid shock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of acid shock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of acid shock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of acid shock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating one or more activities selected from the group consisting of acid shock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of fructose, which comprises
(a) increasing or generating one or more activities selected from the group consisting of phosphotransferase system component in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of phosphotransferase system component in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose, which comprises
(a) increasing or generating one or more activities selected from the group consisting of electron transport complex protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of electron transport complex protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of electron transport complex protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of myo-inositol, which comprises
(a) increasing or generating one or more activities selected from the group consisting of methylglyoxalase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of myo-inositol, which comprises
(a) increasing or generating one or more activities selected from the group consisting of b1670-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating one or more activities selected from the group consisting of ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of myo-inositol, which comprises
(a) increasing or generating one or more activities selected from the group consisting of transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating one or more activities selected from the group consisting of b1796-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of myo-inositol, which comprises
(a) increasing or generating one or more activities selected from the group consisting of para-aminobenzoate synthase component I in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of galactitol, which comprises
(a) increasing or generating one or more activities selected from the group consisting of L-serine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactitol or a composition comprising galactitol in said non-human organism or in the culture medium surrounding said non-human organism.

Accordingly, the present invention relates to a process for the production of sorbitol, which comprises
(a) increasing or generating one or more activities selected from the group consisting of L-serine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sorbitol or a composition comprising sorbitol in said non-human organism or in the culture medium surrounding said non-human organism.

Accordingly, the present invention relates to a process for the production of myo-inositol, which comprises
(a) increasing or generating one or more activities selected from the group consisting of protease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of erythrol, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of erythrol or a composition comprising erythrol in said non-human organism or in the culture medium surrounding said non-human organism.

Accordingly, the present invention relates to a process for the production of myo-inositol, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sorbitol, which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sorbitol or a composition comprising sorbitol in said non-human organism or in the culture medium surrounding said non-human organism.

Accordingly, the present invention relates to a process for the production of fructose, which comprises
(a) increasing or generating one or more activities selected from the group consisting of dATP pyrophosphohydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose-1-phosphate, which comprises
(a) increasing or generating one or more activities selected from the group consisting of trehalose-6-phosphate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of trehalose, which comprises
(a) increasing or generating one or more activities selected from the group consisting of trehalose-6-phosphate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of trehalose or a composition comprising trehalose in said non-human organism or in the culture medium surrounding said non-human organism.

Accordingly, the present invention relates to a process for the production of erythrol, which comprises
(a) increasing or generating one or more activities selected from the group consisting of trehalose-phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of erythrol or a composition comprising erythrol in said non-human organism or in the culture medium surrounding said non-human organism.

Accordingly, the present invention relates to a process for the production of fructose, which comprises
(a) increasing or generating one or more activities selected from the group consisting of trehalose-phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of trehalose-phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of sucrose, which comprises
(a) increasing or generating one or more activities selected from the group consisting of trehalose-phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of raffinose, which comprises
(a) increasing or generating one or more activities selected from the group consisting of b1898-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose-1-phosphate, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose, which comprises
(a) increasing or generating one or more activities selected from the group consisting of flagellar M-ring protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of myo-inositol, which comprises
(a) increasing or generating one or more activities selected from the group consisting of flagellar M-ring protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating one or more activities selected from the group consisting of O-antigen chain length determinant in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of b2032-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of myo-inositol, which comprises
(a) increasing or generating one or more activities selected from the group consisting of b2032-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of glucose-1-phosphate, which comprises
(a) increasing or generating one or more activities selected from the group consisting of colanic acid biosynthesis protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of fucose, which comprises
(a) increasing or generating one or more activities selected from the group consisting of GDP-mannose 4,6-dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fucose or a composition comprising fucose in said non-human organism or in the culture medium surrounding said non-human organism.

Accordingly, the present invention relates to a process for the production of fructose, which comprises
(a) increasing or generating one or more activities selected from the group consisting of transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose-1-phosphate, which comprises
(a) increasing or generating one or more activities selected from the group consisting of transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of raffinose, which comprises
(a) increasing or generating one or more activities selected from the group consisting of transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sucrose, which comprises
(a) increasing or generating one or more activities selected from the group consisting of transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating one or more activities selected from the group consisting of b2107-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of b2107-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating one or more activities selected from the group consisting of b2139-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of erythrol, which comprises
(a) increasing or generating one or more activities selected from the group consisting of sn-glycerol-3-phosphate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of erythrol or a composition comprising erythrol in said non-human organism or in the culture medium surrounding said non-human organism.

Accordingly, the present invention relates to a process for the production of fructose, which comprises
(a) increasing or generating one or more activities selected from the group consisting of ATP synthase subunit E in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of galactinol, which comprises
(a) increasing or generating one or more activities selected from the group consisting of aminotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

Accordingly, the present invention relates to a process for the production of myo-inositol, which comprises
(a) increasing or generating one or more activities selected from the group consisting of b2345-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of b2345-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of verbascose, which comprises
(a) increasing or generating one or more activities selected from the group consisting of b2360-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of verbascose or a composition comprising verbascose in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of b2360-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating one or more activities selected from the group consisting of b2361-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of b2361-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of sucrose, which comprises
(a) increasing or generating one or more activities selected from the group consisting of ion-transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating one or more activities selected from the group consisting of b2399-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of b2399-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of b2399-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of b2399-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating one or more activities selected from the group consisting of b2399-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sedoheptulose-7-phosphate, which comprises
(a) increasing or generating one or more activities selected from the group consisting of b2399-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of sucrose, which comprises
(a) increasing or generating one or more activities selected from the group consisting of b2399-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of raffinose, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of myo-inositol, which comprises
(a) increasing or generating one or more activities selected from the group consisting of cysteine synthase A in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of verbascose, which comprises
(a) increasing or generating one or more activities selected from the group consisting of cysteine synthase A in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of verbascose or a composition comprising verbascose in said non-human organism or in the culture medium surrounding said non-human organism.

Accordingly, the present invention relates to a process for the production of glucose-1-phosphate, which comprises
(a) increasing or generating one or more activities selected from the group consisting of b2466-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of b2466-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of b2466-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating one or more activities selected from the group consisting of b2474-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of raffinose, which comprises
(a) increasing or generating one or more activities selected from the group consisting of 2,3-dihydroxy-2,3-dihydrophenylpropionatedehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sucrose, which comprises
(a) increasing or generating one or more activities selected from the group consisting of putative transport system permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose-1-phosphate, which comprises
(a) increasing or generating one or more activities selected from the group consisting of b2613-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of b2613-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of myo-inositol, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sucrose, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose-1-phosphate, which comprises
(a) increasing or generating one or more activities selected from the group consisting of b2653-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of sedoheptulose-7-phosphate, which comprises
(a) increasing or generating one or more activities selected from the group consisting of b2653-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of b2653-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating one or more activities selected from the group consisting of b2654-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of b2654-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating one or more activities selected from the group consisting of b2670-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating one or more activities selected from the group consisting of b2673-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of b2673-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of myo-inositol, which comprises
(a) increasing or generating one or more activities selected from the group consisting of b2682-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of glucose-1-phosphate, which comprises
(a) increasing or generating one or more activities selected from the group consisting of recombinase A in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating one or more activities selected from the group consisting of murein transglycosylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating one or more activities selected from the group consisting of b2739-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of b2739-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose, which comprises
(a) increasing or generating one or more activities selected from the group consisting of 2-C-methyl-D-erythritol 4-phosphate cytidylyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of myo-inositol, which comprises
(a) increasing or generating one or more activities selected from the group consisting of 2-C-methyl-D-erythritol 4-phosphate cytidylyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating one or more activities selected from the group consisting of 2-C-methyl-D-erythritol 4-phosphate cytidylyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose-1-phosphate, which comprises
(a) increasing or generating one or more activities selected from the group consisting of phosphoadenosine phosphosulfate reductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of phosphoadenosine phosphosulfate reductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating one or more activities selected from the group consisting of flavodoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of flavodoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of flavodoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of flavodoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of flavodoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating one or more activities selected from the group consisting of amino-acid acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose-1-phosphate, which comprises
(a) increasing or generating one or more activities selected from the group consisting of amino-acid acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of amino-acid acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose, which comprises
(a) increasing or generating one or more activities selected from the group consisting of arginine exporter protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of myo-inositol, which comprises
(a) increasing or generating one or more activities selected from the group consisting of arginine exporter protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of myo-inositol, which comprises
(a) increasing or generating one or more activities selected from the group consisting of b2932-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of erythrol, which comprises
(a) increasing or generating one or more activities selected from the group consisting of L-serine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of erythrol or a composition comprising erythrol in said non-human organism or in the culture medium surrounding said non-human organism.

Accordingly, the present invention relates to a process for the production of myo-inositol, which comprises
(a) increasing or generating one or more activities selected from the group consisting of b3151-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating one or more activities selected from the group consisting of b3151-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sucrose, which comprises
(a) increasing or generating one or more activities selected from the group consisting of b3151-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating one or more activities selected from the group consisting of b3221-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of b3221-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of b3221-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of myo-inositol, which comprises
(a) increasing or generating one or more activities selected from the group consisting of b3246-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of myo-inositol, which comprises
(a) increasing or generating one or more activities selected from the group consisting of acetyl CoA carboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating one or more activities selected from the group consisting of methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of raffinose, which comprises
(a) increasing or generating one or more activities selected from the group consisting of methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sucrose, which comprises
(a) increasing or generating one or more activities selected from the group consisting of methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose, which comprises
(a) increasing or generating one or more activities selected from the group consisting of ferredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of myo-inositol, which comprises
(a) increasing or generating one or more activities selected from the group consisting of b3346-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating one or more activities selected from the group consisting of b3346-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of 3,4,5-trihydroxypentanoicacid, which comprises
(a) increasing or generating one or more activities selected from the group consisting of b3346-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating one or more activities selected from the group consisting of phosphoenolpyruvate carboxykinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of phosphoenolpyruvate carboxykinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of B3427-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating one or more activities selected from the group consisting of glycogen synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of glycogen synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of myo-inositol, which comprises
(a) increasing or generating one or more activities selected from the group consisting of b3442-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of B3443-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of saccharic acid, which comprises
(a) increasing or generating one or more activities selected from the group consisting of sn-glycerol-3-phosphate transport system permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

Accordingly, the present invention relates to a process for the production of glucose-1-phosphate, which comprises
(a) increasing or generating one or more activities selected from the group consisting of highaffinity branched-chain amino acid transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of highaffinity branched-chain amino acid transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating one or more activities selected from the group consisting of trehalase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of galactinol, which comprises
(a) increasing or generating one or more activities selected from the group consisting of trehalase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

Accordingly, the present invention relates to a process for the production of fructose, which comprises
(a) increasing or generating one or more activities selected from the group consisting of mannitol-1-phosphate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of mannitol, which comprises
(a) increasing or generating one or more activities selected from the group consisting of mannitol-1-phosphate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of mannitol or a composition comprising mannitol in said non-human organism or in the culture medium surrounding said non-human organism.

Accordingly, the present invention relates to a process for the production of sorbitol, which comprises
(a) increasing or generating one or more activities selected from the group consisting of mannitol-1-phosphate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sorbitol or a composition comprising sorbitol in said non-human organism or in the culture medium surrounding said non-human organism.

Accordingly, the present invention relates to a process for the production of galactinol, which comprises
(a) increasing or generating one or more activities selected from the group consisting of ketol-acid reductoisomerase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

Accordingly, the present invention relates to a process for the production of myo-inositol, which comprises
(a) increasing or generating one or more activities selected from the group consisting of b3777-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of myo-inositol, which comprises
(a) increasing or generating one or more activities selected from the group consisting of b3817-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating one or more activities selected from the group consisting of b3817-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of myo-inositol, which comprises
(a) increasing or generating one or more activities selected from the group consisting of b3818-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of myo-inositol, which comprises
(a) increasing or generating one or more activities selected from the group consisting of threonine efflux protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of myo-inositol, which comprises
(a) increasing or generating one or more activities selected from the group consisting of glutamine synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of verbascose, which comprises
(a) increasing or generating one or more activities selected from the group consisting of glutamine synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of verbascose or a composition comprising verbascose in said non-human organism or in the culture medium surrounding said non-human organism.

Accordingly, the present invention relates to a process for the production of glucose-1-phosphate, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of fructose, which comprises
(a) increasing or generating one or more activities selected from the group consisting of b3989-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of b3989-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of b3989-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of raffinose, which comprises
(a) increasing or generating one or more activities selected from the group consisting of b3989-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sucrose, which comprises
(a) increasing or generating one or more activities selected from the group consisting of b3989-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of galactinol, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

Accordingly, the present invention relates to a process for the production of fructose, which comprises
(a) increasing or generating one or more activities selected from the group consisting of b4029-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of b4029-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of sedoheptulose-7-phosphate, which comprises
(a) increasing or generating one or more activities selected from the group consisting of b4029-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of glucose-1-phosphate, which comprises
(a) increasing or generating one or more activities selected from the group consisting of chorismate lyase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of chorismate lyase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating one or more activities selected from the group consisting of b4050-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of b4050-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of myo-inositol, which comprises
(a) increasing or generating one or more activities selected from the group consisting of b4050-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating one or more activities selected from the group consisting of b4056-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of galactinol, which comprises
(a) increasing or generating one or more activities selected from the group consisting of acetyl-coenzyme A synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

Accordingly, the present invention relates to a process for the production of glucose-1-phosphate, which comprises
(a) increasing or generating one or more activities selected from the group consisting of lipoprotein precursor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of lipoprotein precursor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of myo-inositol, which comprises
(a) increasing or generating one or more activities selected from the group consisting of b4250-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating one or more activities selected from the group consisting of b4250-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of fructose, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of fructose, which comprises
(a) increasing or generating one or more activities selected from the group consisting of gluconate transport system permease 3 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of galactinol, which comprises
(a) increasing or generating one or more activities selected from the group consisting of purine-nucleoside phosphorylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

Accordingly, the present invention relates to a process for the production of myo-inositol, which comprises
(a) increasing or generating one or more activities selected from the group consisting of lysine decarboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sucrose, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose-1-phosphate, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose-1-phosphate, which comprises
(a) increasing or generating one or more activities selected from the group consisting of

GM02LC38418-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of raffinose, which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sucrose, which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of GM02LC46-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of myo-inositol, which comprises
(a) increasing or generating one or more activities selected from the group consisting of alkaline phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of verbascose, which comprises
(a) increasing or generating one or more activities selected from the group consisting of flavodoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of verbascose or a composition comprising verbascose in said non-human organism or in the culture medium surrounding said non-human organism.

Accordingly, the present invention relates to a process for the production of sucrose, which comprises
(a) increasing or generating one or more activities selected from the group consisting of sll0254-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of verbascose, which comprises
(a) increasing or generating one or more activities selected from the group consisting of sll0281-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of verbascose or a composition comprising verbascose in said non-human organism or in the culture medium surrounding said non-human organism.

Accordingly, the present invention relates to a process for the production of myo-inositol, which comprises
(a) increasing or generating one or more activities selected from the group consisting of sll0354-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of myo-inositol, which comprises
(a) increasing or generating one or more activities selected from the group consisting of bifunctional protein (phosphoribosyltransferase and regulatory protein) in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of glucose-1-phosphate, which comprises
(a) increasing or generating one or more activities selected from the group consisting of sll0418-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of sll0418-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of myo-inositol, which comprises
(a) increasing or generating one or more activities selected from the group consisting of sll0418-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of saccharic acid, which comprises
(a) increasing or generating one or more activities selected from the group consisting of sll0418-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of sll0418-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating one or more activities selected from the group consisting of urease subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating one or more activities selected from the group consisting of glucokinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of saccharic acid, which comprises
(a) increasing or generating one or more activities selected from the group consisting of urease accessory protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

Accordingly, the present invention relates to a process for the production of glucose-1-phosphate, which comprises
(a) increasing or generating one or more activities selected from the group consisting of binding-protein-dependent transport systems inner membrane component in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of binding-protein-dependent transport systems inner membrane component in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating one or more activities selected from the group consisting of 3-deoxy-7-phosphoheptulonate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of fructose, which comprises
(a) increasing or generating one or more activities selected from the group consisting of glycogen synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of sucrose, which comprises
(a) increasing or generating one or more activities selected from the group consisting of glycogen synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose-1-phosphate, which comprises
(a) increasing or generating one or more activities selected from the group consisting of coproporphyrinogen oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of short chain dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of raffinose, which comprises
(a) increasing or generating one or more activities selected from the group consisting of glycogen (starch) synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of myo-inositol, which comprises
(a) increasing or generating one or more activities selected from the group consisting of carbamoyl-phosphate synthase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of carbamoyl-phosphate synthase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of myo-inositol, which comprises
(a) increasing or generating one or more activities selected from the group consisting of 4-alpha-glucanotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of alanine dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose, which comprises
(a) increasing or generating one or more activities selected from the group consisting of sll1761-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose, which comprises
(a) increasing or generating one or more activities selected from the group consisting of acyl transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of cation-transporting ATPase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating one or more activities selected from the group consisting of cation-transporting ATPase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of cation-transporting ATPase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating one or more activities selected from the group consisting of cation-transporting ATPase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose-1-phosphate, which comprises
(a) increasing or generating one or more activities selected from the group consisting of glycosidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of fructose, which comprises
(a) increasing or generating one or more activities selected from the group consisting of precorrin methylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of precorrin methylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating one or more activities selected from the group consisting of slr0600-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating one or more activities selected from the group consisting of Glu/Leu/Phe/Val dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating one or more activities selected from the group consisting of malate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of sucrose, which comprises
(a) increasing or generating one or more activities selected from the group consisting of geranylgeranyl pyrophosphate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating one or more activities selected from the group consisting of circadian clock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of circadian clock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of circadian clock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating one or more activities selected from the group consisting of amine oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of fructose, which comprises
(a) increasing or generating one or more activities selected from the group consisting of kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of Mg-protoporphyrin IX monomethyl ester oxidative cyclase 66 kD subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of permease protein of ABC transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of glucose-1-phosphate, which comprises
(a) increasing or generating one or more activities selected from the group consisting of dihydrolipoamide dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of raffinose, which comprises
(a) increasing or generating one or more activities selected from the group consisting of dihydrolipoamide dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of myo-inositol, which comprises
(a) increasing or generating one or more activities selected from the group consisting of slr1107-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of glucose-1-phosphate, which comprises
(a) increasing or generating one or more activities selected from the group consisting of fatty acid desaturase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating one or more activities selected from the group consisting of CDP-diglyceride synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of CDP-diglyceride synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating one or more activities selected from the group consisting of iron(III) dicitrate-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of sucrose, which comprises
(a) increasing or generating one or more activities selected from the group consisting of photosystem I reaction center subunit XI in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating one or more activities selected from the group consisting of photosystem II protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of fructose, which comprises
(a) increasing or generating one or more activities selected from the group consisting of glutamine amidotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of myo-inositol, which comprises
(a) increasing or generating one or more activities selected from the group consisting of glutamine amidotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of glucose-1-phosphate, which comprises
(a) increasing or generating one or more activities selected from the group consisting of NADH dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of raffinose, which comprises
(a) increasing or generating one or more activities selected from the group consisting of pyridoxal phosphate biosynthetic protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose-1-phosphate, which comprises
(a) increasing or generating one or more activities selected from the group consisting of phosphoadenosine phosphosulfate reductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating one or more activities selected from the group consisting of threonine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of threonine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating one or more activities selected from the group consisting of short-chain alcohol dehydrogenase family in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of short-chain alcohol dehydrogenase family in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of short-chain alcohol dehydrogenase family in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of short-chain alcohol dehydrogenase family in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sucrose, which comprises
(a) increasing or generating one or more activities selected from the group consisting of short-chain alcohol dehydrogenase family in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of raffinose, which comprises
(a) increasing or generating one or more activities selected from the group consisting of phosphate acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose-1-phosphate, which comprises
(a) increasing or generating one or more activities selected from the group consisting of photosystem I reaction centre subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of raffinose, which comprises
(a) increasing or generating one or more activities selected from the group consisting of photosystem I reaction centre subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of photosystem I reaction centre subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating one or more activities selected from the group consisting of Sec-independent protein translocase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of Sec-independent protein translocase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of sedoheptulose-7-phosphate, which comprises
(a) increasing or generating one or more activities selected from the group consisting of Sec-independent protein translocase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of fructose, which comprises
(a) increasing or generating one or more activities selected from the group consisting of cell division protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of cell division protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of sucrose, which comprises
(a) increasing or generating one or more activities selected from the group consisting of cell division protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose-1-phosphate, which comprises
(a) increasing or generating one or more activities selected from the group consisting of branched-chain amino acid ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of branched-chain amino acid ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of myo-inositol, which comprises
(a) increasing or generating one or more activities selected from the group consisting of 3-isopropylmalate dehydratase large subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating one or more activities selected from the group consisting of multiple antibiotic resistance protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of multiple antibiotic resistance protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of multiple antibiotic resistance protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of fructose, which comprises
(a) increasing or generating one or more activities selected from the group consisting of TTC1386-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of TTC1386-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of fructose, which comprises
(a) increasing or generating one or more activities selected from the group consisting of homocitrate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose-1-phosphate, which comprises
(a) increasing or generating one or more activities selected from the group consisting of homocitrate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of sucrose, which comprises
(a) increasing or generating one or more activities selected from the group consisting of homocitrate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of raffinose, which comprises
(a) increasing or generating one or more activities selected from the group consisting of permease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of permease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating one or more activities selected from the group consisting of oxidoreductase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating one or more activities selected from the group consisting of XM_{—}473199-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sucrose, which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose-1-phosphate, which comprises
(a) increasing or generating one or more activities selected from the group consisting of 3-deoxy-7-phosphoheptulonate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating one or more activities selected from the group consisting of phosphate permease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of phosphate permease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating one or more activities selected from the group consisting of ycl027w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose-1-phosphate, which comprises
(a) increasing or generating one or more activities selected from the group consisting of ycl027w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of serine/threonine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sucrose, which comprises
(a) increasing or generating one or more activities selected from the group consisting of serine/threonine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating one or more activities selected from the group consisting of phosphoglycerate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of phosphoglycerate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of raffinose, which comprises
(a) increasing or generating one or more activities selected from the group consisting of YCR047W-A-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of ycr102c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of galactinol, which comprises
(a) increasing or generating one or more activities selected from the group consisting of isocitrate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

Accordingly, the present invention relates to a process for the production of myo-inositol, which comprises
(a) increasing or generating one or more activities selected from the group consisting of cell division control protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of myo-inositol, which comprises
(a) increasing or generating one or more activities selected from the group consisting of cyclin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating one or more activities selected from the group consisting of YDL159W-A-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose-1-phosphate, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating one or more activities selected from the group consisting of serine/threonine-protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of integral membrane protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating one or more activities selected from the group consisting of yd1235c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of yd1235c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of yd1235c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of sucrose, which comprises
(a) increasing or generating one or more activities selected from the group consisting of coproporphyrinogen III oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose, which comprises
(a) increasing or generating one or more activities selected from the group consisting of branched-chain amino acid permease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of fructose, which comprises
(a) increasing or generating one or more activities selected from the group consisting of ydr183w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of ydr183w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of ydr507c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of fructose, which comprises
(a) increasing or generating one or more activities selected from the group consisting of 1,4-alpha-glucan branching enzyme in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of 1,4-alpha-glucan branching enzyme in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of YEL030C-A-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5trihydroxypentanoic acid in said non-human organism or in the culture medium surrounding said non-human organism.

Accordingly, the present invention relates to a process for the production of glucose-1-phosphate, which comprises
(a) increasing or generating one or more activities selected from the group consisting of aldolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose, which comprises
(a) increasing or generating one or more activities selected from the group consisting of O-acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of raffinose, which comprises
(a) increasing or generating one or more activities selected from the group consisting of yer177w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of myo-inositol, which comprises
(a) increasing or generating one or more activities selected from the group consisting of YFL019C-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sucrose, which comprises
(a) increasing or generating one or more activities selected from the group consisting of YFL019C-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of sucrose, which comprises
(a) increasing or generating one or more activities selected from the group consisting of hexokinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose-1-phosphate, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating one or more activities selected from the group consisting of ygl096w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of ygl096w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of raffinose, which comprises
(a) increasing or generating one or more activities selected from the group consisting of ygl237c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose-1-phosphate, which comprises
(a) increasing or generating one or more activities selected from the group consisting of alcohol dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of alcohol dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose, which comprises
(a) increasing or generating one or more activities selected from the group consisting of yhl013c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of yhl013c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating one or more activities selected from the group consisting of 3-phosphoglycerate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of fructose, which comprises
(a) increasing or generating one or more activities selected from the group consisting of YIL083C-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of myo-inositol, which comprises
(a) increasing or generating one or more activities selected from the group consisting of DnaJ-like chaperone in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of YJL127W-A-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating one or more activities selected from the group consisting of inositol-3-phosphate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of galactitol, which comprises
(a) increasing or generating one or more activities selected from the group consisting of inositol-3-phosphate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactitol or a composition comprising galactitol in said non-human organism or in the culture medium surrounding said non-human organism.

Accordingly, the present invention relates to a process for the production of myo-inositol, which comprises
(a) increasing or generating one or more activities selected from the group consisting of inositol-3-phosphate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sorbitol, which comprises
(a) increasing or generating one or more activities selected from the group consisting of hexose transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sorbitol or a composition comprising sorbitol in said non-human organism or in the culture medium surrounding said non-human organism.

Accordingly, the present invention relates to a process for the production of myo-inositol, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of glucose, which comprises
(a) increasing or generating one or more activities selected from the group consisting of cystathionine gamma-synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of fructose, which comprises
(a) increasing or generating one or more activities selected from the group consisting of fructose biphosphate aldolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of sedoheptulose-7-phosphate, which comprises
(a) increasing or generating one or more activities selected from the group consisting of fructose biphosphate aldolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of glucose-1-phosphate, which comprises
(a) increasing or generating one or more activities selected from the group consisting of acyl carrier protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of sucrose, which comprises
(a) increasing or generating one or more activities selected from the group consisting of halotolerance protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose, which comprises
(a) increasing or generating one or more activities selected from the group consisting of phosphoenolpyruvate carboxykinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose, which comprises
(a) increasing or generating one or more activities selected from the group consisting of phosphoenolpyruvate carboxykinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of sucrose, which comprises
(a) increasing or generating one or more activities selected from the group consisting of vacuolar sorting protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose-1-phosphate, which comprises
(a) increasing or generating one or more activities selected from the group consisting of thioredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of fructose, which comprises
(a) increasing or generating one or more activities selected from the group consisting of hexose transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of verbascose, which comprises
(a) increasing or generating one or more activities selected from the group consisting of purine biosynthesis protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of verbascose or a composition comprising verbascose in said non-human organism or in the culture medium surrounding said non-human organism.

Accordingly, the present invention relates to a process for the production of glucose-1-phosphate, which comprises
(a) increasing or generating one or more activities selected from the group consisting of ymr324c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of molecular chaperone in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sucrose, which comprises
(a) increasing or generating one or more activities selected from the group consisting of molecular chaperone in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of raffinose, which comprises
(a) increasing or generating one or more activities selected from the group consisting of sterol O-acyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of myo-inositol, which comprises
(a) increasing or generating one or more activities selected from the group consisting of yol160w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of glucose, which comprises
(a) increasing or generating one or more activities selected from the group consisting of phosphohydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of histone acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of subunit of COMPASS-protein complex in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating one or more activities selected from the group consisting of ypl144w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of ypl144w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating one or more activities selected from the group consisting of ypr098c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of ypr098c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating one or more activities selected from the group consisting of polyamine transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of polyamine transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating one or more activities selected from the group consisting of Zm_{—}4842_BE510522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of Zm_{—}4842_BE510522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of myo-inositol, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating one or more activities selected from the group consisting of 47266012-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of 47266012-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of 47266012-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of 47266012-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sedoheptulose-7-phosphate, which comprises
(a) increasing or generating one or more activities selected from the group consisting of 47266012-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of glucose, which comprises
(a) increasing or generating one or more activities selected from the group consisting of 49747384_SOYBEAN-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of 49747384_SOYBEAN-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating one or more activities selected from the group consisting of 49747384_SOYBEAN-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating one or more activities selected from the group consisting of 49747384_SOYBEAN-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sedoheptulose-7-phosphate, which comprises
(a) increasing or generating one or more activities selected from the group consisting of 49747384_SOYBEAN-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of fructose, which comprises
(a) increasing or generating one or more activities selected from the group consisting of glucose-6-phosphate 1-dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of glucose-6-phosphate 1-dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating one or more activities selected from the group consisting of glucose-6-phosphate 1-dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of sedoheptulose-7-phosphate, which comprises
(a) increasing or generating one or more activities selected from the group consisting of glucose-6-phosphate 1-dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of sucrose, which comprises
(a) increasing or generating one or more activities selected from the group consisting of glucose-6-phosphate 1-dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of 3,4,5-trihydroxypentanoic acid, which comprises
(a) increasing or generating one or more activities selected from the group consisting of glucose-6-phosphate 1-dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating one or more activities selected from the group consisting of DNA replication complex GINS protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating one or more activities selected from the group consisting of acetyl CoA synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

An embodiment of the present invention relates to a process for the production of 3,4,5-trihydroxypentanoic acid, which comprises
(a1) increasing or generating the activity of a polypeptide comprising a polypeptide as depicted in a respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical 3,4,5-trihydroxypentanoic acid; or
(a2) increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical 3,4,5-trihydroxypentanoic acid; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of 3,4,5-trihydroxypentanoic acid or a composition comprising 3,4,5-trihydroxypentanoic acid in said non-human organism or in the culture medium surrounding said non-human organism.

An embodiment of the present invention relates to a process for the production of erythrol, which comprises
(a1) increasing or generating the activity of a polypeptide comprising a polypeptide as depicted in a respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical erythrol; or
(a2) increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical erythrol; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of erythrol or a composition comprising erythrol in said non-human organism or in the culture medium surrounding said non-human organism.

An embodiment of the present invention relates to a process for the production of fructose, which comprises
(a1) increasing or generating the activity of a polypeptide comprising a polypeptide as depicted in a respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical fructose; or
(a2) increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical fructose; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of fructose or a composition comprising fructose in said non-human organism or in the culture medium surrounding said non-human organism.

An embodiment of the present invention relates to a process for the production of fucose, which comprises
(a1) increasing or generating the activity of a polypeptide comprising a polypeptide as depicted in a respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical fucose; or
(a2) increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical fucose; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of fucose or a composition comprising fucose in said non-human organism or in the culture medium surrounding said non-human organism.

An embodiment of the present invention relates to a process for the production of galactinol, which comprises
(a1) increasing or generating the activity of a polypeptide comprising a polypeptide as depicted in a respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical galactinol; or
(a2) increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical galactinol; or
(a3) increasing or generating the activity of a functional equivalent of (a1) or (a2); in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of galactinol or a composition comprising galactinol in said non-human organism or in the culture medium surrounding said non-human organism.

An embodiment of the present invention relates to a process for the production of galactitol, which comprises
(a1) increasing or generating the activity of a polypeptide comprising a polypeptide as depicted in a respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical galactitol; or
(a2) increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical galactitol; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of galactitol or a composition comprising galactitol in said non-human organism or in the culture medium surrounding said non-human organism.

An embodiment of the present invention relates to a process for the production of glucose, which comprises
(a1) increasing or generating the activity of a polypeptide comprising a polypeptide as depicted in a respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical glucose; or
(a2) increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical glucose; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of glucose or a composition comprising glucose in said non-human organism or in the culture medium surrounding said non-human organism.

An embodiment of the present invention relates to a process for the production of glucose-1-phosphate, which comprises
(a1) increasing or generating the activity of a polypeptide comprising a polypeptide as depicted in a respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical glucose-1-phosphate; or
(a2) increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical glucose-1-phosphate; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of glucose-1-phosphate or a composition comprising glucose-1-phosphate in said non-human organism or in the culture medium surrounding said non-human organism.

An embodiment of the present invention relates to a process for the production of mannitol, which comprises
(a1) increasing or generating the activity of a polypeptide comprising a polypeptide as depicted in a respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical mannitol; or
(a2) increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical mannitol; or
(a3) increasing or generating the activity of a functional equivalent of (a1) or (a2); in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of mannitol or a composition comprising mannitol in said non-human organism or in the culture medium surrounding said non-human organism.

An embodiment of the present invention relates to a process for the production of melibiose, which comprises
(a1) increasing or generating the activity of a polypeptide comprising a polypeptide as depicted in a respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical melibiose; or
(a2) increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical melibiose; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of melibiose or a composition comprising melibiose in said non-human organism or in the culture medium surrounding said non-human organism.

An embodiment of the present invention relates to a process for the production of myo-inositol, which comprises
(a1) increasing or generating the activity of a polypeptide comprising a polypeptide as depicted in a respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical myo-inositol; or
(a2) increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical myo-inositol; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of myo-inositol or a composition comprising myo-inositol in said non-human organism or in the culture medium surrounding said non-human organism.

An embodiment of the present invention relates to a process for the production of raffinose, which comprises
(a1) increasing or generating the activity of a polypeptide comprising a polypeptide as depicted in a respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical raffinose; or
(a2) increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical raffinose; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of raffinose or a composition comprising raffinose in said non-human organism or in the culture medium surrounding said non-human organism.

An embodiment of the present invention relates to a process for the production of saccharic acid, which comprises
(a1) increasing or generating the activity of a polypeptide comprising a polypeptide as depicted in a respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical saccharic acid; or
(a2) increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical saccharic acid; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of saccharic acid or a composition comprising saccharic acid in said non-human organism or in the culture medium surrounding said non-human organism.

An embodiment of the present invention relates to a process for the production of sedoheptulose-7-phosphate, which comprises
(a1) increasing or generating the activity of a polypeptide comprising a polypeptide as depicted in a respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical sedoheptulose-7-phosphate; or
(a2) increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical sedoheptulose-7-phosphate; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of sedoheptulose-7-phosphate or a composition comprising sedoheptulose-7-phosphate in said non-human organism or in the culture medium surrounding said non-human organism.

An embodiment of the present invention relates to a process for the production of sorbitol, which comprises
(a1) increasing or generating the activity of a polypeptide comprising a polypeptide as depicted in a respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical sorbitol; or
(a2) increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical sorbitol; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of sorbitol or a composition comprising sorbitol in said non-human organism or in the culture medium surrounding said non-human organism.

An embodiment of the present invention relates to a process for the production of sucrose, which comprises
(a1) increasing or generating the activity of a polypeptide comprising a polypeptide as depicted in a respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical sucrose; or
(a2) increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical sucrose; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of sucrose or a composition comprising sucrose in said non-human organism or in the culture medium surrounding said non-human organism.

An embodiment of the present invention relates to a process for the production of trehalose, which comprises
(a1) increasing or generating the activity of a polypeptide comprising a polypeptide as depicted in a respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical trehalose; or
(a2) increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical trehalose; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of trehalose or a composition comprising trehalose in said non-human organism or in the culture medium surrounding said non-human organism.

An embodiment of the present invention relates to a process for the production of verbascose, which comprises
(a1) increasing or generating the activity of a polypeptide comprising a polypeptide as depicted in a respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical verbascose; or
(a2) increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical verbascose; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of verbascose or a composition comprising verbascose in said non-human organism or in the culture medium surrounding said non-human organism.

Accordingly, the increase or generation of one or more said activities is for example conferred by one or more expression products of said nucleic acid molecule, e.g. proteins. Accordingly, in the present invention described above, the increase or generation of one or more said activities is for example conferred by one or more protein(s) each comprising a polypeptide selected from the group as depicted in Table II, application no. 17, column 5 and 8, or a homolog or a fragment thereof.

The process of the invention comprises in one embodiment the following steps:
(i) increasing or generating of the expression of; and/or
(ii) increasing or generating the expression of an expression product of; and/or
(iii) increasing or generating one or more activities of an expression product encoded by;
at least one nucleic acid molecule (in the following “Fine Chemical Related Protein (FCRP)”-encoding gene or “FCRP”-gene) comprising a nucleic acid molecule selected from the group consisting of:
(a) a nucleic acid molecule encoding the polypeptide shown in column 5 or 8 of Table II, preferably Table II B, application no. 17, or a homolog or a fragment thereof;
(b) a nucleic acid molecule shown in column 5 or 8 of Table I, preferably Table I B, application no. 17, or a homolog or a fragment thereof (preferably the coding region thereof);
(c) a nucleic acid molecule, which, as a result of the degeneracy of the genetic code, can be derived from a polypeptide sequence depicted in column 5 or 8 of Table II, preferably Table II B, application no. 17;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 17, or the coding region thereof;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d),
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 17;
(i) a nucleic acid molecule encoding a polypeptide having the activity represented by a respective protein as depicted in column 5 of Table II, application no. 17;
(j) a nucleic acid molecule which comprises a polynucleotide, which is obtained by amplifying a cDNA library or a genomic library using the primers as depicted in column 8 of Table III, application no. 17; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment thereof said nucleic acid molecule encodes a polypeptide which has the activity of the polypeptide represented by a protein comprising a polypeptide as depicted in the corresponding hit in column 5 of Table II, application no. 17.

In another preferred embodiment thereof said nucleic acid molecule confers a generation or an increase of the respective fine chemical in a non-human organism or a part thereof as compared to a corresponding wild-type non-human organism or part thereof.

In another preferred embodiment thereof said nucleic acid molecule encodes a polypeptide which has the activity of the polypeptide represented by a protein as depicted in the corresponding hit in column 5 of Table II, application no. 17, and confers a generation or an increase of the respective fine chemical in a non-human organism or a part thereof as compared to a corresponding wild-type non-human organism or part thereof.

Accordingly, the genes of the present invention or used in accordance with the present invention, which respectively encode a protein having an activity of 1,4-alpha-glucan branching enzyme, 2,3-dihydroxy-2,3-dihydrophenylpropionatedehydrogenase, 2-C-methyl-D-erythritol 4-phosphate cytidylyltransferase, 2-oxoglutarate dehydrogenase E1 subunit, 3-deoxy-7-phosphoheptulonate synthase, 3-isopropylmalate dehydratase large subunit, 3-phosphoglycerate dehydrogenase, 47266012-protein, 49747384_SOYBEAN-protein, 4-alpha-glucanotransferase, ABC transporter permease protein, acetyl CoA carboxylase, acetyl CoA synthetase, acetyl-coenzyme A synthetase, acetyltransferase, acid shock protein, acyl carrier protein, acyl transferase, acyl-CoA dehydrogenase, acyl-CoA synthase, adenylosuccinate lyase, alanine dehydrogenase, alcohol dehydrogenase, aldolase, alkaline phosphatase, amine oxidase, amino-acid acetyltransferase, aminotransferase, arginine exporter protein, asparagine synthetase B, At1g19350-protein, At2g45420-protein, At5g16650-protein, At5g43630-protein, ATP synthase subunit E, ATP-binding component of a transport system, auxin response factor, AX653549-protein, AY087308-protein, b0050-protein, b0251-protein, b0362-protein, b0488-protein, B0644-protein, b0801-protein, b0970-protein, b1003-protein, b1046-protein, b1137-protein, b1214-protein, b1219-protein, b1234-protein, b1285-protein, b1357-protein, b1410-protein, b1425-protein, b1445-protein, b1470-protein, b1522-protein, b1556-protein, b1583-protein, b1670-protein, b1796-protein, b1898-protein, b2032-protein, b2107-protein, b2139-protein, b2345-protein, b2360-protein, b2361-protein, b2399-protein, b2466-protein, b2474-protein, b2613-protein, b2653-protein, b2654-protein, b2670-protein, b2673-protein, b2682-protein, b2739-protein, b2932-protein, b3151-protein, b3221-protein, b3246-protein, b3346-protein, B3427-protein, b3442-protein, B3443-protein, b3777-protein, b3817-protein, b3818-protein, b3989-protein, b4029-protein, b4050-protein, b4056-protein, b4250-protein, beta-galactosidase, beta-hydroxylase, betaine aldehyde dehydrogenase, bifunctional protein (phosphoribosyltransferase and regulatory protein), binding-protein-dependent transport systems inner membrane component, branched-chain amino acid ABC transporter permease protein, branched-chain amino acid permease, calcium-dependent protein kinase, carbamoyl-phosphate synthase subunit, cardiolipin synthase, cation-transporting ATPase, CBL-interacting protein kinase, CCAAT-binding transcription factor, CDP-diglyceride synthetase, cell division control protein, cell division protein, chlorophyllase, chorismate lyase, circadian clock protein, colanic acid biosynthesis protein, coproporphyrinogen III oxidase, coproporphyrinogen oxidase, CTP synthase, cullin, cyclin, cyclin D, cystathionine gamma-synthase, cysteine synthase A, dATP pyrophosphohydrolase, dihydrolipoamide dehydrogenase, DNA binding protein, DNA replication complex GINS protein, DnaJ-like chaperone, electron transfer flavoprotein subunit beta, electron transport complex protein, elongation factor Tu, enoyl-CoA hydratase, eukaryotic translation initiation factor, eukaryotic translation initiation factor 5, fatty acid desaturase, ferredoxin, Fe—S subunit of oxidoreductase, flagellar M-ring protein, flavodoxin, fructose biphosphate aldolase, fumarylacetoacetate hydrolase, galactinol synthase, GDP-mannose 4,6-dehydratase, GDP-mannose dehydrogenase, geranylgeranyl pyrophosphate synthase, gibberellin 20-oxidase, Glu/Leu/Phe/Val dehydrogenase , glucokinase, gluconate transport system permease 3, glucose dehydrogenase, glucose-6-phosphate 1-dehydrogenase, glutamate-ammonia-ligase, glutamine amidotransferase, glutamine synthetase, glutaredoxin, glycerate kinase, glycogen (starch) synthase, glycogen synthase, glycosidase, glycosyl transferase, glycosyltransferase, GM02LC38418-protein, GM02LC46-protein, halotolerance protein, heat shock transcription factor, hexokinase, hexose transporter, high-affinity branched-chain amino acid transport protein, histone acetyltransferase, homocitrate synthase, hydrolase, inositol-3-phosphate synthase, integral membrane protein, intracellular septation protein, ion-transport protein, iron(III) dicitratebinding protein, isochorismate synthase, isocitrate dehydrogenase, isopropylmalate isomerase large subunit, ketol-acid reductoisomerase, kinase, leucyl/phenylalanyl-tRNA-protein transferase, lipoprotein precursor, L-ribulose-5-phosphate 4-epimerase, L-serine dehydratase, lysine decarboxylase, MADS box protein transcription factor, MADS-box transcription factor, malate dehydrogenase, mannitol-1-phosphate dehydrogenase, membrane protein, membrane transport protein, methylglyoxalase, methyltransferase, Mg-protoporphyrin IX monomethyl ester oxidative cyclase 66 kD subunit, mitogen-activated protein kinase, molecular chaperone, monothiol glutaredoxin, monthiol glutaredoxin, multidrug resistance protein, multiple antibiotic resistance protein, murein transglycosylase, NAD(P)H dehydrogenase (quinone), NADH dehydrogenase, NADH-quinone oxidoreductase subunit, O-acetyltransferase, O-antigen chain length determinant, outer membrane receptor protein precursor, oxidoreductase, oxidoreductase subunit, para-aminobenzoate synthase component I, penicillin-binding protein, peptidyl-prolyl cis-trans isomerase, permease, permease protein of ABC transporter, phage-related repressor protein, phenylacetic acid degradation protein, phenylalanine ammonia-lyase, phosphate acetyltransferase, phosphate permease, phosphatidylinositol 3- and 4-kinase family protein, phosphoadenosine phosphosulfate reductase, phosphoanhydride phosphorylase, phosphoenolpyruvate carboxykinase, phosphoglycerate kinase, phosphohydrolase, phosphotransferase system component, photosystem I reaction center subunit XI, photosystem I reaction centre subunit, photosystem II protein, polyamine transporter, precorrin methylase, protease, protein kinase, protein phosphatase, protein-L-isoaspartate O-methyltransferase, purine biosynthesis protein, purine nucleoside phosphorylase, purine-nucleoside phosphorylase, putative transport system permease protein, putrescine transport system permease protein, pyridoxal phosphate biosynthetic protein, pyrimidine-specific ribonucleoside hydrolase, pyruvate kinase, recombinase A, RNA-binding protein, sec-independent protein translocase, Sec-independent protein translocase subunit, sensor histidine kinase, serine protease, serine/threonine dehydratase, serine/threonine-protein phosphatase, short chain dehydrogenase, short-chain alcohol dehydrogenase family, sll0254-protein, sll0281-protein, sll0354-protein, sll0418-protein, sll1761-protein, slr0600-protein, slr1107-protein, sn-glycerol-3-phosphate dehydrogenase, sn-glycerol-3-phosphate transport system permease protein, sodium/proton antiporter, sterol O-acyltransferase, subunit of COMPASS-protein complex, succinyl-CoA synthetase beta chain, ThiF family protein, thioredoxin, thioredoxin family protein, threonine dehydratase, threonine efflux protein, threonine synthase, transcription factor, transcriptional regulator, transcriptional regulator protein, transport protein, trehalase, trehalose-6-phosphate synthase, trehalosephosphatase, tryptophan biosynthesis protein, TTC1386-protein, urease accessory protein, urease subunit, vacuolar sorting protein, XM_{—}473199-protein, ycl027w-protein, YCR047W-A-protein, ycr102c-protein, YDL159W-A-protein, ydl235c-protein, ydr183w-protein, ydr507c-protein, YEL030C-A-protein, yer177w-protein, YFL019C-protein, ygl096w-protein, ygl1237c-protein, yhl013c-protein, YIL083C-protein, YJL127W-A-protein, ymr324c-protein, yol160w-protein, ypl144w-protein, ypr098c-protein, zinc transporter, or Zm_{—}4842_BE510522-protein, which respectively encode a protein comprising a polypeptide encoded by a nucleic acid sequence as shown in Table I, application no. 17, column 5 or 8, (preferably the coding region thereof), or a homolog or a fragment thereof, which respectively encode a protein comprising a polypeptide as depicted in Table II, application no. 17, column 5 or 8, or a homolg or a fragment thereof, and/or which respectively can be amplified with the primer set shown in Table III, application no. 17, column 8, are also referred to as “FCRP genes”.

Proteins or polypeptides encoded by “FCRP-genes” are referred to as “Fine Chemical Related Proteins” or “FCRP”. For the purposes of the description of the present invention, the respective protein having an activity of 1,4-alpha-glucan branching enzyme, 2,3-dihydroxy-2,3-dihydrophenylpropionatedehydrogenase, 2-C-methyl-D-erythritol 4-phosphate cytidylyltransferase, 2-oxoglutarate dehydrogenase E1 subunit, 3-deoxy-7-phosphoheptulonate synthase, 3-isopropylmalate dehydratase large subunit, 3-phosphoglycerate dehydrogenase, 47266012-protein, 49747384_SOYBEAN-protein, 4-alpha-glucanotransferase, ABC transporter permease protein, acetyl CoA carboxylase, acetyl CoA synthetase, acetyl-coenzyme A synthetase, acetyltransferase, acid shock protein, acyl carrier protein, acyl transferase, acyl-CoA dehydrogenase, acyl-CoA synthase, adenylosuccinate lyase, alanine dehydrogenase, alcohol dehydrogenase, aldolase, alkaline phosphatase, amine oxidase, amino-acid acetyltransferase, aminotransferase, arginine exporter protein, asparagine synthetase B, At1g19350-protein, At2g45420-protein, At5g16650-protein, At5g43630-protein, ATP synthase subunit E, ATP-binding component of a transport system, auxin response factor, AX653549-protein, AY087308-protein, b0050-protein, b0251-protein, b0362-protein, b0488-protein, B0644-protein, b0801-protein, b0970-protein, b1003-protein, b1046-protein, b1137-protein, b1214-protein, b1219-protein, b1234-protein, b1285-protein, b1357-protein, b1410-protein, b1425-protein, b1445-protein, b1470-protein, b1522-protein, b1556-protein, b1583-protein, b1670-protein, b1796-protein, b1898-protein, b2032-protein, b2107-protein, b2139-protein, b2345-protein, b2360-protein, b2361-protein, b2399-protein, b2466-protein, b2474-protein, b2613-protein, b2653-protein, b2654-protein, b2670-protein, b2673-protein, b2682-protein, b2739-protein, b2932-protein, b3151-protein, b3221-protein, b3246-protein, b3346-protein, B3427-protein, b3442-protein, B3443-protein, b3777-protein, b3817-protein, b3818-protein, b3989-protein, b4029-protein, b4050-protein, b4056-protein, b4250-protein, beta-galactosidase, beta-hydroxylase, betaine aldehyde dehydrogenase, bifunctional protein (phosphoribosyltransferase and regulatory protein), binding-protein-dependent transport systems inner membrane component, branched-chain amino acid ABC transporter permease protein, branched-chain amino acid permease, calcium-dependent protein kinase, carbamoyl-phosphate synthase subunit, cardiolipin synthase, cation-transporting ATPase, CBL-interacting protein kinase, CCAAT-binding transcription factor, CDP-diglyceride synthetase, cell division control protein, cell division protein, chlorophyllase, chorismate lyase, circadian clock protein, colanic acid biosynthesis protein, coproporphyrinogen III oxidase, coproporphyrinogen oxidase, CTP synthase, cullin, cyclin, cyclin D, cystathionine gamma-synthase, cysteine synthase A, dATP pyrophosphohydrolase, dihydrolipoamide dehydrogenase, DNA binding protein, DNA replication complex GINS protein, DnaJ-like chaperone, electron transfer flavoprotein subunit beta, electron transport complex protein, elongation factor Tu, enoyl-CoA hydratase, eukaryotic translation initiation factor, eukaryotic translation initiation factor 5, fatty acid desaturase, ferredoxin, Fe—S subunit of oxidoreductase, flagellar M-ring protein, flavodoxin, fructose biphosphate aldolase, fumarylacetoacetate hydrolase, galactinol synthase, GDP-mannose 4,6-dehydratase, GDP-mannose dehydrogenase, geranylgeranyl pyrophosphate synthase, gibberellin 20-oxidase, Glu/Leu/PheNal dehydrogenase, glucokinase, gluconate transport system permease 3, glucose dehydrogenase, glucose-6-phosphate 1-dehydrogenase, glutamate-ammonia-ligase, glutamine amidotransferase, glutamine synthetase, glutaredoxin, glycerate kinase, glycogen (starch) synthase, glycogen synthase, glycosidase, glycosyl transferase, glycosyltransferase, GM02LC38418-protein, GM02LC46-protein, halotolerance protein, heat shock transcription factor, hexokinase, hexose transporter, high-affinity branched-chain amino acid transport protein, histone acetyltransferase, homocitrate synthase, hydrolase, inositol-3-phosphate synthase, integral membrane protein, intracellular septation protein, ion-transport protein, iron(III) dicitrate-binding protein, isochorismate synthase, isocitrate dehydrogenase, isopropylmalate isomerase large subunit, ketol-acid reductoisomerase, kinase, leucyl/phenylalanyl-tRNA-protein transferase, lipoprotein precursor, L-ribulose-5-phosphate 4-epimerase, L-serine dehydratase, lysine decarboxylase, MADS box protein transcription factor, MADS-box transcription factor, malate dehydrogenase, mannitol-1-phosphate dehydrogenase, membrane protein, membrane transport protein, methylglyoxalase, methyltransferase, Mg-protoporphyrin IX monomethyl ester oxidative cyclase 66 kD subunit, mitogen-activated protein kinase, molecular chaperone, monothiol glutaredoxin, monthiol glutaredoxin, multidrug resistance protein, multiple antibiotic resistance protein, murein transglycosylase, NAD(P)H dehydrogenase (quinone), NADH dehydrogenase, NADH-quinone oxidoreductase subunit, O-acetyltransferase, O-antigen chain length determinant, outer membrane receptor protein precursor, oxidoreductase, oxidoreductase subunit, para-aminobenzoate synthase component I, penicillin-binding protein, peptidyl-prolyl cis-trans isomerase, permease, permease protein of ABC transporter, phage-related repressor protein, phenylacetic acid degradation protein, phenylalanine ammonia-lyase, phosphate acetyltransferase, phosphate permease, phosphatidylinositol 3- and 4-kinase family protein, phosphoadenosine phosphosulfate reductase, phosphoanhydride phosphorylase, phosphoenolpyruvate carboxykinase, phosphoglycerate kinase, phosphohydrolase, phosphotransferase system component, photosystem I reaction center subunit XI, photosystem I reaction centre subunit, photosystem II protein, polyamine transporter, precorrin methylase, protease, protein kinase, protein phosphatase, protein-L-isoaspartate O-methyltransferase, purine biosynthesis protein, purine nucleoside phosphorylase, purine-nucleoside phosphorylase, putative transport system permease protein, putrescine transport system permease protein, pyridoxal phosphate biosynthetic protein, pyrimidine-specific ribonucleoside hydrolase, pyruvate kinase, recombinase A, RNA-binding protein, sec-independent protein translocase, Sec-independent protein translocase subunit, sensor histidine kinase, serine protease, serine/threonine dehydratase, serine/threonine-protein phosphatase, short chain dehydrogenase, short-chain alcohol dehydrogenase family, sll0254-protein, sll0281-protein, sll0354-protein, sll0418-protein, sll1761-protein, slr0600-protein, slr1107-protein, sn-glycerol-3-phosphate dehydrogenase, sn-glycerol-3-phosphate transport system permease protein, sodium/proton antiporter, sterol O-acyltransferase, subunit of COMPASS-protein complex, succinyl-CoA synthetase beta chain, ThiF family protein, thioredoxin, thioredoxin family protein, threonine dehydratase, threonine efflux protein, threonine synthase, transcription factor, transcriptional regulator, transcriptional regulator protein, transport protein, trehalase, trehalose-6-phosphate synthase, trehalose-phosphatase, tryptophan biosynthesis protein, TTC1386-protein, urease accessory protein, urease subunit, vacuolar sorting protein, XM_{—}473199-protein, ycl027w-protein, YCR047W-A-protein, ycr102c-protein, YDL159W-A-protein, ydl235c-protein, ydr183w-protein, ydr507c-protein, YEL030C-A-protein, yer177w-protein, YFL019C-protein, ygl096w-protein, ygl237c-protein, yh1013c-protein, YIL083C-protein, YJL127W-A-protein, ymr324c-protein, yol160w-protein, ypl144w-protein, ypr098c-protein, zinc transporter, or Zm_{—}4842_BE510522-protein, the respective protein comprising a polypeptide encoded by one or more respective nucleic acid sequences as shown in Table I, application no. 17, column 5 or 8, (preferably the coding region thereof), or a homolog or fragment thereof, the respective protein comprising a respective polypeptide as depicted in Table II, application no. 17, column 5 or 8, or a homolog or fragment thereof, the respective protein comprising a sequence corresponding to the consensus sequence as shown in Table IV, application no. 17, column 8, and/or the respective protein comprising at least one polypeptide motif as shown in Table IV, application no. 17, column 8 is also referred to as Fine Chemical Related Protein” or “FCRP”.

Thus, in one embodiment, the present invention provides a process of the production of 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, and/or verbascose, by increasing or generating one or more activities, especially selected from the group consisting of 1,4-alpha-glucan branching enzyme, 2,3-dihydroxy-2,3-dihydrophenylpropionatedehydrogenase, 2-C-methyl-D-erythritol 4-phosphate cytidylyltransferase, 2-oxoglutarate dehydrogenase E1 subunit, 3-deoxy-7-phosphoheptulonate synthase, 3-isopropylmalate dehydratase large subunit, 3-phosphoglycerate dehydrogenase, 47266012-protein, 49747384_SOYBEAN-protein, 4-alpha-glucanotransferase, ABC transporter permease protein, acetyl CoA carboxylase, acetyl CoA synthetase, acetyl-coenzyme A synthetase, acetyltransferase, acid shock protein, acyl carrier protein, acyl transferase, acyl-CoA dehydrogenase, acyl-CoA synthase, adenylosuccinate lyase, alanine dehydrogenase, alcohol dehydrogenase, aldolase, alkaline phosphatase, amine oxidase, amino-acid acetyltransferase, aminotransferase, arginine exporter protein, asparagine synthetase B, AO g19350-protein, At2g45420-protein, At5g16650-protein, At5g43630-protein, ATP synthase subunit E, ATP-binding component of a transport system, auxin response factor, AX653549-protein, AY087308-protein, b0050-protein, b0251-protein, b0362-protein, b0488-protein, B0644-protein, b0801-protein, b0970-protein, b1003-protein, b1046-protein, b1137-protein, b1214-protein, b1219-protein, b1234-protein, b1285-protein, b1357-protein, b1410-protein, b1425-protein, b1445-protein, b1470-protein, b1522-protein, b1556-protein, b1583-protein, b1670-protein, b1796-protein, b1898-protein, b2032-protein, b2107-protein, b2139-protein, b2345-protein, b2360-protein, b2361-protein, b2399-protein, b2466-protein, b2474-protein, b2613-protein, b2653-protein, b2654-protein, b2670-protein, b2673-protein, b2682-protein, b2739-protein, b2932-protein, b3151-protein, b3221-protein, b3246-protein, b3346-protein, B3427-protein, b3442-protein, B3443-protein, b3777-protein, b3817-protein, b3818-protein, b3989-protein, b4029-protein, b4050-protein, b4056-protein, b4250-protein, beta-galactosidase, beta-hydroxylase, betaine aldehyde dehydrogenase, bifunctional protein (phosphoribosyltransferase and regulatory protein), binding-protein-dependent transport systems inner membrane component, branched-chain amino acid ABC transporter permease protein, branched-chain amino acid permease, calcium-dependent protein kinase, carbamoyl-phosphate synthase subunit, cardiolipin synthase, cation-transporting ATPase, CBL-interacting protein kinase, CCAAT-binding transcription factor, CDP-diglyceride synthetase, cell division control protein, cell division protein, chlorophyllase, chorismate lyase, circadian clock protein, colanic acid biosynthesis protein, coproporphyrinogen III oxidase, coproporphyrinogen oxidase, CTP synthase, cullin, cyclin, cyclin D, cystathionine gamma-synthase, cysteine synthase A, dATP pyrophosphohydrolase, dihydrolipoamide dehydrogenase, DNA binding protein, DNA replication complex GINS protein, DnaJ-like chaperone, electron transfer flavoprotein subunit beta, electron transport complex protein, elongation factor Tu, enoyl-CoA hydratase, eukaryotic translation initiation factor, eukaryotic translation initiation factor 5, fatty acid desaturase, ferredoxin, Fe—S subunit of oxidoreductase, flagellar M-ring protein, flavodoxin, fructose biphosphate aldolase, fumarylacetoacetate hydrolase, galactinol synthase, GDP-mannose 4,6-dehydratase, GDP-mannose dehydrogenase, geranylgeranyl pyrophosphate synthase, gibberellin 20-oxidase, Glu/Leu/PheNal dehydrogenase , glucokinase, gluconate transport system permease 3, glucose dehydrogenase, glucose-6-phosphate 1-dehydrogenase, glutamate-ammonia-ligase, glutamine amidotransferase, glutamine synthetase, glutaredoxin, glycerate kinase, glycogen (starch) synthase, glycogen synthase, glycosidase, glycosyl transferase, glycosyltransferase, GM02LC38418-protein, GM02LC46-protein, halotolerance protein, heat shock transcription factor, hexokinase, hexose transporter, high-affinity branched-chain amino acid transport protein, histone acetyltransferase, homocitrate synthase, hydrolase, inositol-3-phosphate synthase, integral membrane protein, intracellular septation protein, ion-transport protein, iron(III) dicitratebinding protein, isochorismate synthase, isocitrate dehydrogenase, isopropylmalate isomerase large subunit, ketol-acid reductoisomerase, kinase, leucyl/phenylalanyl-tRNA-protein transferase, lipoprotein precursor, L-ribulose-5-phosphate 4-epimerase, L-serine dehydratase, lysine decarboxylase, MADS box protein transcription factor, MADS-box transcription factor, malate dehydrogenase, mannitol-1-phosphate dehydrogenase, membrane protein, membrane transport protein, methylglyoxalase, methyltransferase, Mg-protoporphyrin IX monomethyl ester oxidative cyclase 66 kD subunit, mitogen-activated protein kinase, molecular chaperone, monothiol glutaredoxin, monthiol glutaredoxin, multidrug resistance protein, multiple antibiotic resistance protein, murein transglycosylase, NAD(P)H dehydrogenase (quinone), NADH dehydrogenase, NADH-quinone oxidoreductase subunit, O-acetyltransferase, O-antigen chain length determinant, outer membrane receptor protein precursor, oxidoreductase, oxidoreductase subunit, para-aminobenzoate synthase component I, penicillin-binding protein, peptidyl-prolyl cis-trans isomerase, permease, permease protein of ABC transporter, phage-related repressor protein, phenylacetic acid degradation protein, phenylalanine ammonia-lyase, phosphate acetyltransferase, phosphate permease, phosphatidylinositol 3- and 4-kinase family protein, phosphoadenosine phosphosulfate reductase, phosphoanhydride phosphorylase, phosphoenolpyruvate carboxykinase, phosphoglycerate kinase, phosphohydrolase, phosphotransferase system component, photosystem I reaction center subunit XI, photosystem I reaction centre subunit, photosystem II protein, polyamine transporter, precorrin methylase, protease, protein kinase, protein phosphatase, protein-L-isoaspartate 0-methyltransferase, purine biosynthesis protein, purine nucleoside phosphorylase, purine-nucleoside phosphorylase, putative transport system permease protein, putrescine transport system permease protein, pyridoxal phosphate biosynthetic protein, pyrimidine-specific ribonucleoside hydrolase, pyruvate kinase, recombinase A, RNA-binding protein, sec-independent protein translocase, Sec-independent protein translocase subunit, sensor histidine kinase, serine protease, serine/threonine dehydratase, serine/threonine-protein phosphatase, short chain dehydrogenase, short-chain alcohol dehydrogenase family, sll0254-protein, sll0281-protein, sll0354-protein, sll0418-protein, sll1761-protein, slr0600-protein, slr1107-protein, sn-glycerol-3-phosphate dehydrogenase, sn-glycerol-3-phosphate transport system permease protein, sodium/proton antiporter, sterol O-acyltransferase, subunit of COMPASS-protein complex, succinyl-CoA synthetase beta chain,

ThiF family protein, thioredoxin, thioredoxin family protein, threonine dehydratase, threonine efflux protein, threonine synthase, transcription factor, transcriptional regulator, transcriptional regulator protein, transport protein, trehalase, trehalose-6-phosphate synthase, trehalosephosphatase, tryptophan biosynthesis protein, TTC1386-protein, urease accessory protein, urease subunit, vacuolar sorting protein, XM_{—}473199-protein, ycl027w-protein, YCR047W-A-protein, ycr102c-protein, YDL159W-A-protein, ydl235c-protein, ydr183w-protein, ydr507c-protein, YEL030C-A-protein, yer177w-protein, YFL019C-protein, ygl096w-protein, ygl237c-protein, yh1013c-protein, YIL083C-protein, YJL127W-A-protein, ymr324c-protein, yol160w-protein, ypl144w-protein, ypr098c-protein, zinc transporter, and Zm_{—}4842_BE510522-protein, which is conferred by one or more FCRPs or the gene product of one or more FCRP-genes, for example by the gene product of a nucleic acid sequences comprising a polynucleotide selected from the group as shown in Table I, application no. 17, column 5 or 8, (preferably by the coding region thereof), or a homolog or a fragment thereof, e.g. or by one or more proteins each comprising a polypeptide encoded by one or more nucleic acid sequences selected from the group as shown in Table I, application no. 17, column 5 or 8, (preferably by the coding region thereof), or a homolog or a fragment thereof, or by one or more protein(s) each comprising a polypeptide selected from the group as depicted in Table II, application no. 17, column 5 and 8, or a homolog thereof, or a protein comprising a sequence corresponding to the consensus sequence or comprising at least one polypeptide motif as shown in Table IV, application no. 17, column 8.

for the disclosure of this paragraph see [0025.1.7.7] above

In an embodiment, the process comprises increasing or generating the activity of one or more polypeptides having said activity, e.g. by generating or increasing the amount and/or specific activity in the cell or a compartment of a cell of one of more FCRP, especially selected from the group consisting of 1,4-alpha-glucan branching enzyme, 2,3-dihydroxy-2,3-dihydrophenylpropionatedehydrogenase, 2-C-methyl-D-erythritol 4-phosphate cytidylyltransferase, 2-oxoglutarate dehydrogenase E1 subunit, 3-deoxy-7-phosphoheptulonate synthase, 3-isopropylmalate dehydratase large subunit, 3-phosphoglycerate dehydrogenase, 47266012-protein, 49747384_SOYBEAN-protein, 4-alpha-glucanotransferase, ABC transporter permease protein, acetyl CoA carboxylase, acetyl CoA synthetase, acetyl-coenzyme A synthetase, acetyltransferase, acid shock protein, acyl carrier protein, acyl transferase, acyl-CoA dehydrogenase, acyl-CoA synthase, adenylosuccinate lyase, alanine dehydrogenase, alcohol dehydrogenase, aldolase, alkaline phosphatase, amine oxidase, amino-acid acetyltransferase, aminotransferase, arginine exporter protein, asparagine synthetase B, At1g19350-protein, At2g45420-protein, At5g16650-protein, At5g43630-protein, ATP synthase subunit E, ATP-binding component of a transport system, auxin response factor, AX653549-protein, AY087308-protein, b0050-protein, b0251-protein, b0362-protein, b0488-protein, B0644-protein, b0801-protein, b0970-protein, b1003-protein, b1046-protein, b1137-protein, b1214-protein, b1219-protein, b1234-protein, b1285-protein, b1357-protein, b1410-protein, b1425-protein, b1445-protein, b1470-protein, b1522-protein, b1556-protein, b1583-protein, b1670-protein, b1796-protein, b1898-protein, b2032-protein, b2107-protein, b2139-protein, b2345-protein, b2360-protein, b2361-protein, b2399-protein, b2466-protein, b2474-protein, b2613-protein, b2653-protein, b2654-protein, b2670-protein, b2673-protein, b2682-protein, b2739-protein, b2932-protein, b3151-protein, b3221-protein, b3246-protein, b3346-protein, B3427-protein, b3442-protein, B3443-protein, b3777-protein, b3817-protein, b3818-protein, b3989-protein, b4029-protein, b4050-protein, b4056-protein, b4250-protein, beta-galactosidase, beta-hydroxylase, betaine aldehyde dehydrogenase, bifunctional protein (phosphoribosyltransferase and regulatory protein), binding-protein-dependent transport systems inner membrane component, branched-chain amino acid ABC transporter permease protein, branched-chain amino acid permease, calcium-dependent protein kinase, carbamoyl-phosphate synthase subunit, cardiolipin synthase, cation-transporting ATPase, CBL-interacting protein kinase, CCAAT-binding transcription factor, CDP-diglyceride synthetase, cell division control protein, cell division protein, chlorophyllase, chorismate lyase, circadian clock protein, colanic acid biosynthesis protein, coproporphyrinogen III oxidase, coproporphyrinogen oxidase, CTP synthase, cullin, cyclin, cyclin D, cystathionine gamma-synthase, cysteine synthase A, dATP pyrophosphohydrolase, dihydrolipoamide dehydrogenase, DNA binding protein, DNA replication complex GINS protein, DnaJ-like chaperone, electron transfer flavoprotein subunit beta, electron transport complex protein, elongation factor Tu, enoyl-CoA hydratase, eukaryotic translation initiation factor, eukaryotic translation initiation factor 5, fatty acid desaturase, ferredoxin, Fe—S subunit of oxidoreductase, flagellar M-ring protein, flavodoxin, fructose biphosphate aldolase, fumarylacetoacetate hydrolase, galactinol synthase, GDP-mannose 4,6-dehydratase, GDP-mannose dehydrogenase, geranylgeranyl pyrophosphate synthase, gibberellin 20-oxidase, Glu/Leu/Phe/Val dehydrogenase , glucokinase, gluconate transport system permease 3, glucose dehydrogenase, glucose-6-phosphate 1-dehydrogenase, glutamate-ammonia-ligase, glutamine amidotransferase, glutamine synthetase, glutaredoxin, glycerate kinase, glycogen (starch) synthase, glycogen synthase, glycosidase, glycosyl transferase, glycosyltransferase, GM02LC38418-protein, GM02LC46-protein, halotolerance protein, heat shock transcription factor, hexokinase, hexose transporter, high-affinity branched-chain amino acid transport protein, histone acetyltransferase, homocitrate synthase, hydrolase, inositol-3-phosphate synthase, integral membrane protein, intracellular septation protein, ion-transport protein, iron(III) dicitratebinding protein, isochorismate synthase, isocitrate dehydrogenase, isopropylmalate isomerase large subunit, ketol-acid reductoisomerase, kinase, leucyl/phenylalanyl-tRNA-protein transferase, lipoprotein precursor, L-ribulose-5-phosphate 4-epimerase, L-serine dehydratase, lysine decarboxylase, MADS box protein transcription factor, MADS-box transcription factor, malate dehydrogenase, mannitol-1-phosphate dehydrogenase, membrane protein, membrane transport protein, methylglyoxalase, methyltransferase, Mg-protoporphyrin IX monomethyl ester oxidative cyclase 66 kD subunit, mitogen-activated protein kinase, molecular chaperone, monothiol glutaredoxin, monthiol glutaredoxin, multidrug resistance protein, multiple antibiotic resistance protein, murein transglycosylase, NAD(P)H dehydrogenase (quinone), NADH dehydrogenase, NADH-quinone oxidoreductase subunit, O-acetyltransferase, O-antigen chain length determinant, outer membrane receptor protein precursor, oxidoreductase, oxidoreductase subunit, para-aminobenzoate synthase component I, penicillin-binding protein, peptidyl-prolyl cis-trans isomerase, permease, permease protein of ABC transporter, phage-related repressor protein, phenylacetic acid degradation protein, phenylalanine ammonia-lyase, phosphate acetyltransferase, phosphate permease, phosphatidylinositol 3- and 4-kinase family protein, phosphoadenosine phosphosulfate reductase, phosphoanhydride phosphorylase, phosphoenolpyruvate carboxykinase, phosphoglycerate kinase, phosphohydrolase, phosphotransferase system component, photosystem I reaction center subunit XI, photosystem I reaction centre subunit, photosystem II protein, polyamine transporter, precorrin methylase, protease, protein kinase, protein phosphatase, protein-L-isoaspartate 0-methyltransferase, purine biosynthesis protein, purine nucleoside phosphorylase, purine-nucleoside phosphorylase, putative transport system permease protein, putrescine transport system permease protein, pyridoxal phosphate biosynthetic protein, pyrimidine-specific ribonucleoside hydrolase, pyruvate kinase, recombinase A, RNA-binding protein, sec-independent protein translocase, Sec-independent protein translocase subunit, sensor histidine kinase, serine protease, serine/threonine dehydratase, serine/threonine-protein phosphatase, short chain dehydrogenase, short-chain alcohol dehydrogenase family, sll0254-protein, sll0281-protein, sll0354-protein, sll0418-protein, sll1761-protein, slr0600-protein, slr1107-protein, sn-glycerol-3-phosphate dehydrogenase, sn-glycerol-3-phosphate transport system permease protein, sodium/proton antiporter, sterol Oacyltransferase, subunit of COMPASS-protein complex, succinyl-CoA synthetase beta chain,

ThiF family protein, thioredoxin, thioredoxin family protein, threonine dehydratase, threonine efflux protein, threonine synthase, transcription factor, transcriptional regulator, transcriptional regulator protein, transport protein, trehalase, trehalose-6-phosphate synthase, trehalosephosphatase, tryptophan biosynthesis protein, TTC1386-protein, urease accessory protein, urease subunit, vacuolar sorting protein, XM_{—}473199-protein, ycl027w-protein, YCR047W-A-protein, ycr102c-protein, YDL159W-A-protein, ydl235c-protein, ydr183w-protein, ydr507c-protein, YEL030C-A-protein, yer177w-protein, YFL019C-protein, ygl096w-protein, ygl237c-protein, yh1013c-protein, YIL083C-protein, YJL127W-A-protein, ymr324c-protein, yol160w-protein, ypl144w-protein, ypr098c-protein, zinc transporter, and Zm_{—}4842_BE510522-protein, for example of the respective polypeptide as depicted in Table II, application no. 17, column 5 and 8, or a homolog or a fragment thereof, or the respective polypeptide comprising a sequence corresponding to the consensus sequences as shown in Table IV, application no. 17, column 8, or the respective polypeptide comprising at least one polypeptide motif as depicted in Table IV, application no. 17, column 8.

A further embodiment of the present invention relates to a process for the production of raffinose, which comprises
(a) increasing or generating the activity of a galactinol synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose-1-phosphate, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of glucose-1-phosphate, which comprises
(a) increasing or generating the activity of a At1g19350-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a At1g19350-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of myo-inositol, which comprises
(a) increasing or generating the activity of a chlorophyllase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating the activity of a cullin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of fructose, which comprises
(a) increasing or generating the activity of a eukaryotic translation initiation factor 5 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a eukaryotic translation initiation factor 5 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of raffinose, which comprises
(a) increasing or generating the activity of a eukaryotic translation initiation factor 5 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sucrose, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of raffinose, which comprises
(a) increasing or generating the activity of a galactinol synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sucrose, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of saccharic acid, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

A further embodiment of the present invention relates to a process for the production of fructose, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of saccharic acid, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

A further embodiment of the present invention relates to a process for the production of fructose, which comprises
(a) increasing or generating the activity of a CBL-interacting protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a CBL-interacting protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of sedoheptulose-7-phosphate, which comprises
(a) increasing or generating the activity of a CBL-interacting protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of glucose-1-phosphate, which comprises
(a) increasing or generating the activity of a phenylalanine ammonia-lyase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating the activity of a MADS box protein transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a At2g45420-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of glucose-1-phosphate, which comprises
(a) increasing or generating the activity of a phosphatidylinositol 3- and 4-kinase family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of raffinose, which comprises
(a) increasing or generating the activity of a galactinol synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of raffinose, which comprises
(a) increasing or generating the activity of a galactinol synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of fructose, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of myo-inositol, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of fructose, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of myo-inositol, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating the activity of a thioredoxin family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a thioredoxin family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of myo-inositol, which comprises
(a) increasing or generating the activity of a thioredoxin family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of saccharic acid, which comprises
(a) increasing or generating the activity of a DNA binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

A further embodiment of the present invention relates to a process for the production of glucose-1-phosphate, which comprises
(a) increasing or generating the activity of a CCAAT-binding transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of raffinose, which comprises
(a) increasing or generating the activity of a CCAAT-binding transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of fructose, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of sedoheptulose-7-phosphate, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of fructose, which comprises
(a) increasing or generating the activity of a eukaryotic translation initiation factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a eukaryotic translation initiation factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of raffinose, which comprises
(a) increasing or generating the activity of a eukaryotic translation initiation factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of myo-inositol, which comprises
(a) increasing or generating the activity of a mitogen-activated protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sedoheptulose-7-phosphate, which comprises
(a) increasing or generating the activity of a auxin response factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of fructose, which comprises
(a) increasing or generating the activity of a monthiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a monthiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a monthiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a monthiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sedoheptulose-7-phosphate, which comprises
(a) increasing or generating the activity of a monthiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of sucrose, which comprises
(a) increasing or generating the activity of a monthiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sedoheptulose-7-phosphate, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of sucrose, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose-1-phosphate, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of fructose, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sedoheptulose-7-phosphate, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of sucrose, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sedoheptulose-7-phosphate, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of sucrose, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sucrose, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of sedoheptulose-7-phosphate, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of sucrose, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of myo-inositol, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sedoheptulose-7-phosphate, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of sucrose, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose-1-phosphate, which comprises
(a) increasing or generating the activity of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of raffinose, which comprises
(a) increasing or generating the activity of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sucrose, which comprises
(a) increasing or generating the activity of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose-1-phosphate, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of raffinose, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of myo-inositol, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sucrose, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of myo-inositol, which comprises
(a) increasing or generating the activity of a gibberellin 20-oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of myo-inositol, which comprises
(a) increasing or generating the activity of a inositol-3-phosphate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating the activity of a inositol-3-phosphate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of fructose, which comprises
(a) increasing or generating the activity of a At5g16650-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of fructose, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of myo-inositol, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sedoheptulose-7-phosphate, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of sucrose, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose-1-phosphate, which comprises
(a) increasing or generating the activity of a At5g43630-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose-1-phosphate, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating the activity of a RNA-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a RNA-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of fructose, which comprises
(a) increasing or generating the activity of a MADS-box transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of raffinose, which comprises
(a) increasing or generating the activity of a MADS-box transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of saccharic acid, which comprises
(a) increasing or generating the activity of a MADS-box transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

A further embodiment of the present invention relates to a process for the production of glucose-1-phosphate, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of raffinose, which comprises
(a) increasing or generating the activity of a glycosyl transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sucrose, which comprises
(a) increasing or generating the activity of a sec-independent protein translocase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of 3,4,5-trihydroxypentanoic acid, which comprises
(a) increasing or generating the activity of a beta-hydroxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating the activity of a elongation factor Tu non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a elongation factor Tu non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of fructose, which comprises
(a) increasing or generating the activity of a ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of sucrose, which comprises
(a) increasing or generating the activity of a ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose-1-phosphate, which comprises
(a) increasing or generating the activity of a acyl-CoA synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose, which comprises
(a) increasing or generating the activity of a 2-oxoglutarate dehydrogenase E1 subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a 2-oxoglutarate dehydrogenase E1 subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a 2-oxoglutarate dehydrogenase E1 subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating the activity of a 2-oxoglutarate dehydrogenase E1 subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sedoheptulose-7-phosphate, which comprises
(a) increasing or generating the activity of a 2-oxoglutarate dehydrogenase E1 subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of raffinose, which comprises
(a) increasing or generating the activity of a GDP-mannose dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of fructose, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a ThiF family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating the activity of a CTP synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a CTP synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of sucrose, which comprises
(a) increasing or generating the activity of a CTP synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of raffinose, which comprises
(a) increasing or generating the activity of a protein-L-isoaspartate O-methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of myo-inositol, which comprises
(a) increasing or generating the activity of a recombinase A non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating the activity of a recombinase A non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of myo-inositol, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of raffinose, which comprises
(a) increasing or generating the activity of a enoyl-CoA hydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of fructose, which comprises
(a) increasing or generating the activity of a NADH-quinone oxidoreductase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose-1-phosphate, which comprises
(a) increasing or generating the activity of a NADH-quinone oxidoreductase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of sucrose, which comprises
(a) increasing or generating the activity of a NADH-quinone oxidoreductase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of 3,4,5-trihydroxypentanoic acid, which comprises
(a) increasing or generating the activity of a isopropylmalate isomerase large subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating the activity of a hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of sucrose, which comprises
(a) increasing or generating the activity of a hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating the activity of a glutamate-ammonia-ligase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a glutamate-ammonia-ligase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of sucrose, which comprises
(a) increasing or generating the activity of a glutamate-ammonia-ligase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating the activity of a fumarylacetoacetate hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of raffinose, which comprises
(a) increasing or generating the activity of a glycosyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of myo-inositol, which comprises
(a) increasing or generating the activity of a peptidyl-prolyl cis-trans isomerase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of 3,4,5-trihydroxypentanoic acid, which comprises
(a) increasing or generating the activity of a NAD(P)H dehydrogenase (quinone) non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating the activity of a electron transfer flavoprotein subunit beta non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a electron transfer flavoprotein subunit beta non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a purine nucleoside phosphorylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a purine nucleoside phosphorylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of raffinose, which comprises
(a) increasing or generating the activity of a transcriptional regulator protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose-1-phosphate, which comprises
(a) increasing or generating the activity of a AX653549-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating the activity of a AY087308-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a AY087308-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of glucose-1-phosphate, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose, which comprises
(a) increasing or generating the activity of a threonine synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of sucrose, which comprises
(a) increasing or generating the activity of a threonine synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of 3,4,5-trihydroxypentanoic acid, which comprises
(a) increasing or generating the activity of a b0050-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of raffinose, which comprises
(a) increasing or generating the activity of a L-ribulose-5-phosphate 4-epimerase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sucrose, which comprises
(a) increasing or generating the activity of a glucose dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose-1-phosphate, which comprises
(a) increasing or generating the activity of a serine protease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a serine protease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sucrose, which comprises
(a) increasing or generating the activity of a acyl-CoA dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose-1-phosphate, which comprises
(a) increasing or generating the activity of a b0251-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of fructose, which comprises
(a) increasing or generating the activity of a betaine aldehyde dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of melibiose, which comprises
(a) increasing or generating the activity of a beta-galactosidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of melibiose or a composition comprising melibiose in said non-human organism or in the culture medium surrounding said non-human organism.

A further embodiment of the present invention relates to a process for the production of glucose-1-phosphate, which comprises
(a) increasing or generating the activity of a b0362-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating the activity of a penicillin-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of fructose, which comprises
(a) increasing or generating the activity of a ATP-binding component of a transport system non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a ATP-binding component of a transport system non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose, which comprises
(a) increasing or generating the activity of a membrane transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of saccharic acid, which comprises
(a) increasing or generating the activity of a b0488-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

A further embodiment of the present invention relates to a process for the production of galactinol, which comprises
(a) increasing or generating the activity of a glycerate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

A further embodiment of the present invention relates to a process for the production of fructose, which comprises
(a) increasing or generating the activity of a isochorismate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a isochorismate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of raffinose, which comprises
(a) increasing or generating the activity of a isochorismate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sucrose, which comprises
(a) increasing or generating the activity of a isochorismate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of 3,4,5-trihydroxypentanoic acid, which comprises
(a) increasing or generating the activity of a B0644-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of saccharic acid, which comprises
(a) increasing or generating the activity of a pyrimidine-specific ribonucleoside hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

A further embodiment of the present invention relates to a process for the production of erythrol, which comprises
(a) increasing or generating the activity of a asparagine synthetase B non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of erythrol or a composition comprising erythrol in said non-human organism or in the culture medium surrounding said non-human organism.

A further embodiment of the present invention relates to a process for the production of myo-inositol, which comprises
(a) increasing or generating the activity of a sensor histidine kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of myo-inositol, which comprises
(a) increasing or generating the activity of a succinyl-CoA synthetase beta chain non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating the activity of a zinc transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a zinc transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a zinc transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of raffinose, which comprises
(a) increasing or generating the activity of a zinc transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose, which comprises
(a) increasing or generating the activity of a b0801-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of sedoheptulose-7-phosphate, which comprises
(a) increasing or generating the activity of a putrescine transport system permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of raffinose, which comprises
(a) increasing or generating the activity of a leucyl/phenylalanyl-tRNA-protein transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose-1-phosphate, which comprises
(a) increasing or generating the activity of a b0970-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a phosphoanhydride phosphorylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating the activity of a b1003-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a b1003-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a b1003-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of sedoheptulose-7-phosphate, which comprises
(a) increasing or generating the activity of a b1003-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of sucrose, which comprises
(a) increasing or generating the activity of a b1003-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of 3,4,5-trihydroxypentanoic acid, which comprises
(a) increasing or generating the activity of a b1046-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating the activity of a multidrug resistance protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of sucrose, which comprises
(a) increasing or generating the activity of a multidrug resistance protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating the activity of a outer membrane receptor protein precursor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose-1-phosphate, which comprises
(a) increasing or generating the activity of a adenylosuccinate lyase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose, which comprises
(a) increasing or generating the activity of a isocitrate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a b1137-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating the activity of a sodium/proton antiporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating the activity of a b1214-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a b1214-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating the activity of a b1219-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a b1219-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of 3,4,5-trihydroxypentanoic acid, which comprises
(a) increasing or generating the activity of a b1219-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of myo-inositol, which comprises
(a) increasing or generating the activity of a b1234-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of galactinol, which comprises
(a) increasing or generating the activity of a cardiolipin synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

A further embodiment of the present invention relates to a process for the production of galactitol, which comprises
(a) increasing or generating the activity of a cardiolipin synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactitol or a composition comprising galactitol in said non-human organism or in the culture medium surrounding said non-human organism.

A further embodiment of the present invention relates to a process for the production of sorbitol, which comprises
(a) increasing or generating the activity of a cardiolipin synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sorbitol or a composition comprising sorbitol in said non-human organism or in the culture medium surrounding said non-human organism.

A further embodiment of the present invention relates to a process for the production of 3,4,5-trihydroxypentanoic acid, which comprises
(a) increasing or generating the activity of a intracellular septation protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating the activity of a membrane protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a membrane protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of raffinose, which comprises
(a) increasing or generating the activity of a membrane protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sucrose, which comprises
(a) increasing or generating the activity of a membrane protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of myo-inositol, which comprises
(a) increasing or generating the activity of a tryptophan biosynthesis protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating the activity of a b1285-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose-1-phosphate, which comprises
(a) increasing or generating the activity of a phage-related repressor protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating the activity of a b1357-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose, which comprises
(a) increasing or generating the activity of a phenylacetic acid degradation protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of 3,4,5-trihydroxypentanoic acid, which comprises
(a) increasing or generating the activity of a phenylacetic acid degradation protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of sedoheptulose-7-phosphate, which comprises
(a) increasing or generating the activity of a b1410-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of myo-inositol, which comprises
(a) increasing or generating the activity of a b1425-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of glucose-1-phosphate, which comprises
(a) increasing or generating the activity of a lipoprotein precursor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of sedoheptulose-7-phosphate, which comprises
(a) increasing or generating the activity of a lipoprotein precursor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a lipoprotein precursor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of 3,4,5-trihydroxypentanoic acid, which comprises
(a) increasing or generating the activity of a b1445-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating the activity of a b1470-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of fructose, which comprises
(a) increasing or generating the activity of a b1522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose-1-phosphate, which comprises
(a) increasing or generating the activity of a b1522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating the activity of a b1556-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of sedoheptulose-7-phosphate, which comprises
(a) increasing or generating the activity of a b1556-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of glucose-1-phosphate, which comprises
(a) increasing or generating the activity of a b1583-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating the activity of a Fe—S subunit of oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a Fe—S subunit of oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating the activity of a acid shock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a acid shock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a acid shock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a acid shock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating the activity of a acid shock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of fructose, which comprises
(a) increasing or generating the activity of a phosphotransferase system component non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a phosphotransferase system component non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose, which comprises
(a) increasing or generating the activity of a electron transport complex protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a electron transport complex protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a electron transport complex protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of myo-inositol, which comprises
(a) increasing or generating the activity of a methylglyoxalase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of myo-inositol, which comprises
(a) increasing or generating the activity of a b1670-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating the activity of a ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of myo-inositol, which comprises
(a) increasing or generating the activity of a transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating the activity of a b1796-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of myo-inositol, which comprises
(a) increasing or generating the activity of a para-aminobenzoate synthase component I non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of galactitol, which comprises
(a) increasing or generating the activity of a L-serine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactitol or a composition comprising galactitol in said non-human organism or in the culture medium surrounding said non-human organism.

A further embodiment of the present invention relates to a process for the production of sorbitol, which comprises
(a) increasing or generating the activity of a L-serine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sorbitol or a composition comprising sorbitol in said non-human organism or in the culture medium surrounding said non-human organism.

A further embodiment of the present invention relates to a process for the production of myo-inositol, which comprises
(a) increasing or generating the activity of a protease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of erythrol, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of erythrol or a composition comprising erythrol in said non-human organism or in the culture medium surrounding said non-human organism.

A further embodiment of the present invention relates to a process for the production of myo-inositol, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sorbitol, which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sorbitol or a composition comprising sorbitol in said non-human organism or in the culture medium surrounding said non-human organism.

A further embodiment of the present invention relates to a process for the production of fructose, which comprises
(a) increasing or generating the activity of a dATP pyrophosphohydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose-1-phosphate, which comprises
(a) increasing or generating the activity of a trehalose-6-phosphate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of trehalose, which comprises
(a) increasing or generating the activity of a trehalose-6-phosphate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of trehalose or a composition comprising trehalose in said non-human organism or in the culture medium surrounding said non-human organism.

A further embodiment of the present invention relates to a process for the production of erythrol, which comprises
(a) increasing or generating the activity of a trehalose-phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of erythrol or a composition comprising erythrol in said non-human organism or in the culture medium surrounding said non-human organism.

A further embodiment of the present invention relates to a process for the production of fructose, which comprises
(a) increasing or generating the activity of a trehalose-phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a trehalose-phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of sucrose, which comprises
(a) increasing or generating the activity of a trehalose-phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of raffinose, which comprises
(a) increasing or generating the activity of a b1898-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose-1-phosphate, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose, which comprises
(a) increasing or generating the activity of a flagellar M-ring protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of myo-inositol, which comprises
(a) increasing or generating the activity of a flagellar M-ring protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating the activity of a O-antigen chain length determinant non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a b2032-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of myo-inositol, which comprises
(a) increasing or generating the activity of a b2032-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of glucose-1-phosphate, which comprises
(a) increasing or generating the activity of a colanic acid biosynthesis protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of fucose, which comprises
(a) increasing or generating the activity of a GDP-mannose 4,6-dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fucose or a composition comprising fucose in said non-human organism or in the culture medium surrounding said non-human organism.

A further embodiment of the present invention relates to a process for the production of fructose, which comprises
(a) increasing or generating the activity of a transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose-1-phosphate, which comprises
(a) increasing or generating the activity of a transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of raffinose, which comprises
(a) increasing or generating the activity of a transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sucrose, which comprises
(a) increasing or generating the activity of a transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of 3,4,5-trihydroxypentanoic acid, which comprises
(a) increasing or generating the activity of a transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating the activity of a b2107-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a b2107-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating the activity of a b2139-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of erythrol, which comprises
(a) increasing or generating the activity of a sn-glycerol-3-phosphate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of erythrol or a composition comprising erythrol in said non-human organism or in the culture medium surrounding said non-human organism.

A further embodiment of the present invention relates to a process for the production of fructose, which comprises
(a) increasing or generating the activity of a ATP synthase subunit E non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of galactinol, which comprises
(a) increasing or generating the activity of a aminotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

A further embodiment of the present invention relates to a process for the production of myo-inositol, which comprises
(a) increasing or generating the activity of a b2345-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of 3,4,5-trihydroxypentanoic acid, which comprises
(a) increasing or generating the activity of a b2345-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of verbascose, which comprises
(a) increasing or generating the activity of a b2360-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of verbascose or a composition comprising verbascose in said non-human organism or in the culture medium surrounding said non-human organism.

A further embodiment of the present invention relates to a process for the production of 3,4,5-trihydroxypentanoic acid, which comprises
(a) increasing or generating the activity of a b2360-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating the activity of a b2361-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a b2361-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of sucrose, which comprises
(a) increasing or generating the activity of a ion-transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating the activity of a b2399-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a b2399-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a b2399-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a b2399-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating the activity of a b2399-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sedoheptulose-7-phosphate, which comprises
(a) increasing or generating the activity of a b2399-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of sucrose, which comprises
(a) increasing or generating the activity of a b2399-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of raffinose, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of myo-inositol, which comprises
(a) increasing or generating the activity of a cysteine synthase A non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of verbascose, which comprises
(a) increasing or generating the activity of a cysteine synthase A non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of verbascose or a composition comprising verbascose in said non-human organism or in the culture medium surrounding said non-human organism.

A further embodiment of the present invention relates to a process for the production of glucose-1-phosphate, which comprises
(a) increasing or generating the activity of a b2466-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a b2466-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of 3,4,5-trihydroxypentanoic acid, which comprises
(a) increasing or generating the activity of a b2466-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating the activity of a b2474-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of raffinose, which comprises
(a) increasing or generating the activity of a 2,3-dihydroxy-2,3-dihydrophenylpropionatedehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sucrose, which comprises
(a) increasing or generating the activity of a putative transport system permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose-1-phosphate, which comprises
(a) increasing or generating the activity of a b2613-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a b2613-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of myo-inositol, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sucrose, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose-1-phosphate, which comprises
(a) increasing or generating the activity of a b2653-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of sedoheptulose-7-phosphate, which comprises
(a) increasing or generating the activity of a b2653-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a b2653-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating the activity of a b2654-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a b2654-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating the activity of a b2670-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating the activity of a b2673-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a b2673-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of myo-inositol, which comprises
(a) increasing or generating the activity of a b2682-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of glucose-1-phosphate, which comprises
(a) increasing or generating the activity of a recombinase A non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating the activity of a murein transglycosylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating the activity of a b2739-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a b2739-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose, which comprises
(a) increasing or generating the activity of a 2-C-methyl-D-erythritol 4-phosphate cytidylyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of myo-inositol, which comprises
(a) increasing or generating the activity of a 2-C-methyl-D-erythritol 4-phosphate cytidylyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating the activity of a 2-C-methyl-D-erythritol 4-phosphate cytidylyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose-1-phosphate, which comprises
(a) increasing or generating the activity of a phosphoadenosine phosphosulfate reductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a phosphoadenosine phosphosulfate reductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating the activity of a flavodoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a flavodoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a flavodoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a flavodoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of 3,4,5-trihydroxypentanoic acid, which comprises
(a) increasing or generating the activity of a flavodoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating the activity of a amino-acid acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose-1-phosphate, which comprises
(a) increasing or generating the activity of a amino-acid acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a amino-acid acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose, which comprises
(a) increasing or generating the activity of a arginine exporter protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of myo-inositol, which comprises
(a) increasing or generating the activity of a arginine exporter protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of myo-inositol, which comprises
(a) increasing or generating the activity of a b2932-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of erythrol, which comprises
(a) increasing or generating the activity of a L-serine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of erythrol or a composition comprising erythrol in said non-human organism or in the culture medium surrounding said non-human organism.

A further embodiment of the present invention relates to a process for the production of myo-inositol, which comprises
(a) increasing or generating the activity of a b3151-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating the activity of a b3151-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sucrose, which comprises
(a) increasing or generating the activity of a b3151-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating the activity of a b3221-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a b3221-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of 3,4,5-trihydroxypentanoic acid, which comprises
(a) increasing or generating the activity of a b3221-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of myo-inositol, which comprises
(a) increasing or generating the activity of a b3246-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of myo-inositol, which comprises
(a) increasing or generating the activity of a acetyl CoA carboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating the activity of a methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of raffinose, which comprises
(a) increasing or generating the activity of a methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sucrose, which comprises
(a) increasing or generating the activity of a methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose, which comprises
(a) increasing or generating the activity of a ferredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of myo-inositol, which comprises
(a) increasing or generating the activity of a b3346-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating the activity of a b3346-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of 3,4,5-trihydroxypentanoic acid, which comprises
(a) increasing or generating the activity of a b3346-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating the activity of a phosphoenolpyruvate carboxykinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a phosphoenolpyruvate carboxykinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of 3,4,5-trihydroxypentanoic acid, which comprises
(a) increasing or generating the activity of a B3427-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating the activity of a glycogen synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a glycogen synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of myo-inositol, which comprises
(a) increasing or generating the activity of a b3442-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of 3,4,5-trihydroxypentanoic acid, which comprises
(a) increasing or generating the activity of a B3443-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of saccharic acid, which comprises
(a) increasing or generating the activity of a sn-glycerol-3-phosphate transport system permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

A further embodiment of the present invention relates to a process for the production of glucose-1-phosphate, which comprises
(a) increasing or generating the activity of a high-affinity branched-chain amino acid transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a high-affinity branched-chain amino acid transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating the activity of a trehalase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of galactinol, which comprises
(a) increasing or generating the activity of a trehalase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

A further embodiment of the present invention relates to a process for the production of fructose, which comprises
(a) increasing or generating the activity of a mannitol-1-phosphate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of mannitol, which comprises
(a) increasing or generating the activity of a mannitol-1-phosphate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of mannitol or a composition comprising mannitol in said non-human organism or in the culture medium surrounding said non-human organism.

A further embodiment of the present invention relates to a process for the production of sorbitol, which comprises
(a) increasing or generating the activity of a mannitol-1-phosphate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sorbitol or a composition comprising sorbitol in said non-human organism or in the culture medium surrounding said non-human organism.

A further embodiment of the present invention relates to a process for the production of galactinol, which comprises
(a) increasing or generating the activity of a ketol-acid reductoisomerase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

A further embodiment of the present invention relates to a process for the production of myo-inositol, which comprises
(a) increasing or generating the activity of a b3777-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of myo-inositol, which comprises
(a) increasing or generating the activity of a b3817-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating the activity of a b3817-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of myo-inositol, which comprises
(a) increasing or generating the activity of a b3818-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of myo-inositol, which comprises
(a) increasing or generating the activity of a threonine efflux protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of myo-inositol, which comprises
(a) increasing or generating the activity of a glutamine synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of verbascose, which comprises
(a) increasing or generating the activity of a glutamine synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of verbascose or a composition comprising verbascose in said non-human organism or in the culture medium surrounding said non-human organism.

A further embodiment of the present invention relates to a process for the production of glucose-1-phosphate, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of fructose, which comprises
(a) increasing or generating the activity of a b3989-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a b3989-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a b3989-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of raffinose, which comprises
(a) increasing or generating the activity of a b3989-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sucrose, which comprises
(a) increasing or generating the activity of a b3989-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of galactinol, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

A further embodiment of the present invention relates to a process for the production of fructose, which comprises
(a) increasing or generating the activity of a b4029-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a b4029-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of sedoheptulose-7-phosphate, which comprises
(a) increasing or generating the activity of a b4029-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of glucose-1-phosphate, which comprises
(a) increasing or generating the activity of a chorismate lyase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a chorismate lyase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating the activity of a b4050-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a b4050-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of myo-inositol, which comprises
(a) increasing or generating the activity of a b4050-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating the activity of a b4056-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of galactinol, which comprises
(a) increasing or generating the activity of a acetyl-coenzyme A synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

A further embodiment of the present invention relates to a process for the production of glucose-1-phosphate, which comprises
(a) increasing or generating the activity of a lipoprotein precursor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a lipoprotein precursor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of myo-inositol, which comprises
(a) increasing or generating the activity of a b4250-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating the activity of a b4250-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of fructose, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of fructose, which comprises
(a) increasing or generating the activity of a gluconate transport system permease 3 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of galactinol, which comprises
(a) increasing or generating the activity of a purine-nucleoside phosphorylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

A further embodiment of the present invention relates to a process for the production of myo-inositol, which comprises
(a) increasing or generating the activity of a lysine decarboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sucrose, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose-1-phosphate, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose-1-phosphate, which comprises
(a) increasing or generating the activity of a GM02LC38418-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating the activity of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of raffinose, which comprises
(a) increasing or generating the activity of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sucrose, which comprises
(a) increasing or generating the activity of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of 3,4,5-trihydroxypentanoic acid, which comprises
(a) increasing or generating the activity of a GM02LC46-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of 3,4,5-trihydroxypentanoic acid, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of myo-inositol, which comprises
(a) increasing or generating the activity of a alkaline phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of verbascose, which comprises
(a) increasing or generating the activity of a flavodoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of verbascose or a composition comprising verbascose in said non-human organism or in the culture medium surrounding said non-human organism.

A further embodiment of the present invention relates to a process for the production of sucrose, which comprises
(a) increasing or generating the activity of a sll0254-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of verbascose, which comprises
(a) increasing or generating the activity of a sll0281-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of verbascose or a composition comprising verbascose in said non-human organism or in the culture medium surrounding said non-human organism.

A further embodiment of the present invention relates to a process for the production of myo-inositol, which comprises
(a) increasing or generating the activity of a sll0354-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of myo-inositol, which comprises
(a) increasing or generating the activity of a bifunctional protein (phosphoribosyltransferase and regulatory protein) non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of glucose-1-phosphate, which comprises
(a) increasing or generating the activity of a sll0418-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a sll0418-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of myo-inositol, which comprises
(a) increasing or generating the activity of a sll0418-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of saccharic acid, which comprises
(a) increasing or generating the activity of a sll0418-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

A further embodiment of the present invention relates to a process for the production of 3,4,5-trihydroxypentanoic acid, which comprises
(a) increasing or generating the activity of a sll0418-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating the activity of a urease subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating the activity of a glucokinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of saccharic acid, which comprises
(a) increasing or generating the activity of a urease accessory protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

A further embodiment of the present invention relates to a process for the production of glucose-1-phosphate, which comprises
(a) increasing or generating the activity of a binding-protein-dependent transport systems inner membrane component non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a binding-protein-dependent transport systems inner membrane component non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating the activity of a 3-deoxy-7-phosphoheptulonate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of fructose, which comprises
(a) increasing or generating the activity of a glycogen synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of sucrose, which comprises
(a) increasing or generating the activity of a glycogen synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose-1-phosphate, which comprises
(a) increasing or generating the activity of a coproporphyrinogen oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a short chain dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of raffinose, which comprises
(a) increasing or generating the activity of a glycogen (starch) synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of myo-inositol, which comprises
(a) increasing or generating the activity of a carbamoyl-phosphate synthase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of 3,4,5-trihydroxypentanoic acid, which comprises
(a) increasing or generating the activity of a carbamoyl-phosphate synthase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of myo-inositol, which comprises
(a) increasing or generating the activity of a 4-alpha-glucanotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of 3,4,5-trihydroxypentanoic acid, which comprises
(a) increasing or generating the activity of a alanine dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose, which comprises
(a) increasing or generating the activity of a sll1761-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose, which comprises
(a) increasing or generating the activity of a acyl transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a cation-transporting ATPase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating the activity of a cation-transporting ATPase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a cation-transporting ATPase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating the activity of a cation-transporting ATPase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose-1-phosphate, which comprises
(a) increasing or generating the activity of a glycosidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of fructose, which comprises
(a) increasing or generating the activity of a precorrin methylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a precorrin methylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating the activity of a slr0600-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating the activity of a Glu/Leu/PheNal dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating the activity of a malate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of sucrose, which comprises
(a) increasing or generating the activity of a geranylgeranyl pyrophosphate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating the activity of a circadian clock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a circadian clock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a circadian clock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating the activity of a amine oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of fructose, which comprises
(a) increasing or generating the activity of a kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a Mg-protoporphyrin IX monomethyl ester oxidative cyclase 66 kD subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a permease protein of ABC transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of glucose-1-phosphate, which comprises
(a) increasing or generating the activity of a dihydrolipoamide dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of raffinose, which comprises
(a) increasing or generating the activity of a dihydrolipoamide dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of myo-inositol, which comprises
(a) increasing or generating the activity of a slr1107-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of glucose-1-phosphate, which comprises
(a) increasing or generating the activity of a fatty acid desaturase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating the activity of a CDP-diglyceride synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a CDP-diglyceride synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating the activity of a iron(III) dicitrate-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of sucrose, which comprises
(a) increasing or generating the activity of a photosystem I reaction center subunit XI non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating the activity of a photosystem II protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of fructose, which comprises
(a) increasing or generating the activity of a glutamine amidotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of myo-inositol, which comprises
(a) increasing or generating the activity of a glutamine amidotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of glu-cose-1-phosphate, which comprises
(a) increasing or generating the activity of a NADH dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of raffinose, which comprises
(a) increasing or generating the activity of a pyridoxal phosphate biosynthetic protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose-1-phosphate, which comprises
(a) increasing or generating the activity of a phosphoadenosine phosphosulfate reductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating the activity of a threonine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a threonine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating the activity of a short-chain alcohol dehydrogenase family non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a short-chain alcohol dehydrogenase family non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a short-chain alcohol dehydrogenase family non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a short-chain alcohol dehydrogenase family non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sucrose, which comprises
(a) increasing or generating the activity of a short-chain alcohol dehydrogenase family non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of raffinose, which comprises
(a) increasing or generating the activity of a phosphate acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose-1-phosphate, which comprises
(a) increasing or generating the activity of a photosystem I reaction centre subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of raffinose, which comprises
(a) increasing or generating the activity of a photosystem I reaction centre subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of 3,4,5-trihydroxypentanoic acid, which comprises
(a) increasing or generating the activity of a photosystem I reaction centre subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating the activity of a Sec-independent protein translocase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a Sec-independent protein translocase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of sedoheptulose-7-phosphate, which comprises
(a) increasing or generating the activity of a Sec-independent protein translocase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of fructose, which comprises
(a) increasing or generating the activity of a cell division protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a cell division protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of sucrose, which comprises
(a) increasing or generating the activity of a cell division protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose-1-phosphate, which comprises
(a) increasing or generating the activity of a branched-chain amino acid ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a branched-chain amino acid ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of myo-inositol, which comprises
(a) increasing or generating the activity of a 3-isopropylmalate dehydratase large subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating the activity of a multiple antibiotic resistance protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a multiple antibiotic resistance protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a multiple antibiotic resistance protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of fructose, which comprises
(a) increasing or generating the activity of a TTC1386-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a TTC1386-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of fructose, which comprises
(a) increasing or generating the activity of a homocitrate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose-1-phosphate, which comprises
(a) increasing or generating the activity of a homocitrate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of sucrose, which comprises
(a) increasing or generating the activity of a homocitrate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of raffinose, which comprises
(a) increasing or generating the activity of a permease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of 3,4,5-trihydroxypentanoic acid, which comprises
(a) increasing or generating the activity of a permease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating the activity of a oxidoreductase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating the activity of a XM_{—}473199-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sucrose, which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose-1-phosphate, which comprises
(a) increasing or generating the activity of a 3-deoxy-7-phosphoheptulonate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating the activity of a phosphate permease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a phosphate permease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating the activity of a ycl027w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose-1-phosphate, which comprises
(a) increasing or generating the activity of a ycl027w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a serine/threonine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sucrose, which comprises
(a) increasing or generating the activity of a serine/threonine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating the activity of a phosphoglycerate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a phosphoglycerate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of raffinose, which comprises
(a) increasing or generating the activity of a YCR047W-A-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of 3,4,5-trihydroxypentanoic acid, which comprises
(a) increasing or generating the activity of a ycr102c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of galactinol, which comprises
(a) increasing or generating the activity of a isocitrate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

A further embodiment of the present invention relates to a process for the production of myo-inositol, which comprises
(a) increasing or generating the activity of a cell division control protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of myo-inositol, which comprises
(a) increasing or generating the activity of a cyclin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating the activity of a YDL159W-A-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose-1-phosphate, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating the activity of a serine/threonine-protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a integral membrane protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating the activity of a ydl235c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a ydl235c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of 3,4,5-trihydroxypentanoic acid, which comprises
(a) increasing or generating the activity of a ydl235c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of sucrose, which comprises
(a) increasing or generating the activity of a coproporphyrinogen III oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose, which comprises
(a) increasing or generating the activity of a branched-chain amino acid permease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of fructose, which comprises
(a) increasing or generating the activity of a ydr183w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a ydr183w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a ydr507c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of fructose, which comprises
(a) increasing or generating the activity of a 1,4-alpha-glucan branching enzyme non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a 1,4-alpha-glucan branching enzyme non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of 3,4,5-trihydroxypentanoic acid, which comprises
(a) increasing or generating the activity of a YEL030C-A-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating the activity of a aldolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose, which comprises
(a) increasing or generating the activity of a O-acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of raffinose, which comprises
(a) increasing or generating the activity of a yer177w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of myo-inositol, which comprises
(a) increasing or generating the activity of a YFL019C-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sucrose, which comprises
(a) increasing or generating the activity of a YFL019C-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of sucrose, which comprises
(a) increasing or generating the activity of a hexokinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose-1-phosphate, which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating the activity of a ygl096w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a ygl096w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of raffinose, which comprises
(a) increasing or generating the activity of a ygl237c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose-1-phosphate, which comprises
(a) increasing or generating the activity of a alcohol dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a alcohol dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose, which comprises
(a) increasing or generating the activity of a yhl013c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a yhl013c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating the activity of a 3-phosphoglycerate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of fructose, which comprises
(a) increasing or generating the activity of a YIL083C-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of myo-inositol, which comprises
(a) increasing or generating the activity of a DnaJ-like chaperone non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of 3,4,5-trihydroxypentanoic acid, which comprises
(a) increasing or generating the activity of a YJL127W-A-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating the activity of a inositol-3-phosphate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of galactitol, which comprises
(a) increasing or generating the activity of a inositol-3-phosphate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactitol or a composition comprising galactitol in said non-human organism or in the culture medium surrounding said non-human organism.

A further embodiment of the present invention relates to a process for the production of myo-inositol, which comprises
(a) increasing or generating the activity of a inositol-3-phosphate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sorbitol, which comprises
(a) increasing or generating the activity of a hexose transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sorbitol or a composition comprising sorbitol in said non-human organism or in the culture medium surrounding said non-human organism.

A further embodiment of the present invention relates to a process for the production of myo-inositol, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of glucose, which comprises
(a) increasing or generating the activity of a cystathionine gamma-synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of fructose, which comprises
(a) increasing or generating the activity of a fructose biphosphate aldolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of sedoheptulose-7-phosphate, which comprises
(a) increasing or generating the activity of a fructose biphosphate aldolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of glucose-1-phosphate, which comprises
(a) increasing or generating the activity of a acyl carrier protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of sucrose, which comprises
(a) increasing or generating the activity of a halotolerance protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose, which comprises
(a) increasing or generating the activity of a phosphoenolpyruvate carboxykinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose, which comprises
(a) increasing or generating the activity of a phosphoenolpyruvate carboxykinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of sucrose, which comprises
(a) increasing or generating the activity of a vacuolar sorting protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose-1-phosphate, which comprises
(a) increasing or generating the activity of a thioredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of fructose, which comprises
(a) increasing or generating the activity of a hexose transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of verbascose, which comprises
(a) increasing or generating the activity of a purine biosynthesis protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of verbascose or a composition comprising verbascose in said non-human organism or in the culture medium surrounding said non-human organism.

A further embodiment of the present invention relates to a process for the production of glucose-1-phosphate, which comprises
(a) increasing or generating the activity of a ymr324c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a molecular chaperone non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sucrose, which comprises
(a) increasing or generating the activity of a molecular chaperone non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of raffinose, which comprises
(a) increasing or generating the activity of a sterol O-acyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of myo-inositol, which comprises
(a) increasing or generating the activity of a yol160w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of glucose, which comprises
(a) increasing or generating the activity of a phosphohydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a histone acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a subunit of COMPASS-protein complex non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating the activity of a ypl144w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a ypl144w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating the activity of a ypr098c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating the activity of a ypr098c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating the activity of a polyamine transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a polyamine transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating the activity of a Zm_{—}4842_BE510522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a Zm_{—}4842_BE510522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of myo-inositol, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating the activity of a 47266012-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a 47266012-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a 47266012-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a 47266012-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sedoheptulose-7-phosphate, which comprises
(a) increasing or generating the activity of a 47266012-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of glucose, which comprises
(a) increasing or generating the activity of a 49747384_SOYBEAN-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a 49747384_SOYBEAN-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating the activity of a 49747384_SOYBEAN-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating the activity of a 49747384_SOYBEAN-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sedoheptulose-7-phosphate, which comprises
(a) increasing or generating the activity of a 49747384_SOYBEAN-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of fructose, which comprises
(a) increasing or generating the activity of a glucose-6-phosphate 1-dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a glucose-6-phosphate 1-dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating the activity of a glucose-6-phosphate 1-dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of sedoheptulose-7-phosphate, which comprises
(a) increasing or generating the activity of a glucose-6-phosphate 1-dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of sucrose, which comprises
(a) increasing or generating the activity of a glucose-6-phosphate 1-dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of 3,4,5-trihydroxypentanoic acid, which comprises
(a) increasing or generating the activity of a glucose-6-phosphate 1-dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating the activity of a DNA replication complex GINS protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating the activity of a acetyl CoA synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of 3,4,5-trihydroxypentanoic acid, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical 3,4,5-trihydroxypentanoic acid; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical 3,4,5-trihydroxypentanoic acid;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of 3,4,5-trihydroxypentanoic acid, or a composition comprising 3,4,5-trihydroxypentanoic acid in said non-human organism or in the culture medium surrounding said non-human organism.

A further embodiment of the present invention relates to a process for the production of erythrol, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical erythrol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical erythrol;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of erythrol, or a composition comprising erythrol in said non-human organism or in the culture medium surrounding said non-human organism.

A further embodiment of the present invention relates to a process for the production of fructose, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical fructose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical fructose;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of fructose, or a composition comprising fructose in said non-human organism or in the culture medium surrounding said non-human organism.

A further embodiment of the present invention relates to a process for the production of fucose, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical fucose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical fucose;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of fucose, or a composition comprising fucose in said non-human organism or in the culture medium surrounding said non-human organism.

A further embodiment of the present invention relates to a process for the production of galactinol, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical galactinol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical galactinol;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of galactinol, or a composition comprising galactinol in said non-human organism or in the culture medium surrounding said non-human organism.

A further embodiment of the present invention relates to a process for the production of galactitol, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical galactitol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical galactitol;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of galactitol, or a composition comprising galactitol in said non-human organism or in the culture medium surrounding said non-human organism.

A further embodiment of the present invention relates to a process for the production of glucose, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical glucose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical glucose;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of glucose, or a composition comprising glucose in said non-human organism or in the culture medium surrounding said non-human organism.

A further embodiment of the present invention relates to a process for the production of glucose-1-phosphate, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical glucose-1-phosphate; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical glucose-1-phosphate;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of glucose-1-phosphate, or a composition comprising glucose-1-phosphate in said non-human organism or in the culture medium surrounding said non-human organism.

A further embodiment of the present invention relates to a process for the production of mannitol, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical mannitol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical mannitol;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of mannitol, or a composition comprising mannitol in said non-human organism or in the culture medium surrounding said non-human organism.

A further embodiment of the present invention relates to a process for the production of melibiose, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical melibiose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical melibiose;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of melibiose, or a composition comprising melibiose in said non-human organism or in the culture medium surrounding said non-human organism.

A further embodiment of the present invention relates to a process for the production of myo-inositol, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical myo-inositol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical myo-inositol;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of myo-inositol, or a composition comprising myo-inositol in said non-human organism or in the culture medium surrounding said non-human organism.

A further embodiment of the present invention relates to a process for the production of raffinose, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical raffinose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical raffinose;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of raffinose, or a composition comprising raffinose in said non-human organism or in the culture medium surrounding said non-human organism.

A further embodiment of the present invention relates to a process for the production of saccharic acid, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical saccharic acid; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical saccharic acid;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of saccharic acid, or a composition comprising saccharic acid in said non-human organism or in the culture medium surrounding said non-human organism.

A further embodiment of the present invention relates to a process for the production of sedoheptulose-7-phosphate, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical sedoheptulose-7-phosphate; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical sedoheptulose-7-phosphate;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of sedoheptulose-7-phosphate, or a composition comprising sedoheptulose-7-phosphate in said non-human organism or in the culture medium surrounding said non-human organism.

A further embodiment of the present invention relates to a process for the production of sorbitol, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical sorbitol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical sorbitol;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of sorbitol, or a composition comprising sorbitol in said non-human organism or in the culture medium surrounding said non-human organism.

A further embodiment of the present invention relates to a process for the production of sucrose, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical sucrose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical sucrose;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of sucrose, or a composition comprising sucrose in said non-human organism or in the culture medium surrounding said non-human organism.

A further embodiment of the present invention relates to a process for the production of trehalose, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical trehalose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical trehalose;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of trehalose, or a composition comprising trehalose in said non-human organism or in the culture medium surrounding said non-human organism.

A further embodiment of the present invention relates to a process for the production of verbascose, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical verbascose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical verbascose;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of verbascose, or a composition comprising verbascose in said non-human organism or in the culture medium surrounding said non-human organism.

A further embodiment of the present invention relates to a process for the production of raffinose, which comprises
(a) increasing or generating one or more activities selected from the group consisting of galactinol synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose-1-phosphate, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of glucose-1-phosphate, which comprises
(a) increasing or generating one or more activities selected from the group consisting of At1g19350-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of At1g19350-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of myo-inositol, which comprises
(a) increasing or generating one or more activities selected from the group consisting of chlorophyllase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating one or more activities selected from the group consisting of cullin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of fructose, which comprises
(a) increasing or generating one or more activities selected from the group consisting of eukaryotic translation initiation factor 5 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of eukaryotic translation initiation factor 5 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of raffinose, which comprises
(a) increasing or generating one or more activities selected from the group consisting of eukaryotic translation initiation factor 5 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sucrose, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of raffinose, which comprises
(a) increasing or generating one or more activities selected from the group consisting of galactinol synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sucrose, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of saccharic acid, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

A further embodiment of the present invention relates to a process for the production of fructose, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of saccharic acid, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

A further embodiment of the present invention relates to a process for the production of fructose, which comprises
(a) increasing or generating one or more activities selected from the group consisting of CBL-interacting protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of CBL-interacting protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of sedoheptulose-7-phosphate, which comprises
(a) increasing or generating one or more activities selected from the group consisting of CBL-interacting protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of glucose-1-phosphate, which comprises
(a) increasing or generating one or more activities selected from the group consisting of phenylalanine ammonia-lyase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating one or more activities selected from the group consisting of MADS box protein transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of At2g45420-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of glucose-1-phosphate, which comprises
(a) increasing or generating one or more activities selected from the group consisting of phosphatidylinositol 3- and 4-kinase family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of raffinose, which comprises
(a) increasing or generating one or more activities selected from the group consisting of galactinol synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of raffinose, which comprises
(a) increasing or generating one or more activities selected from the group consisting of galactinol synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of fructose, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of myo-inositol, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of fructose, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of myo-inositol, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating one or more activities selected from the group consisting of thioredoxin family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of thioredoxin family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of myo-inositol, which comprises
(a) increasing or generating one or more activities selected from the group consisting of thioredoxin family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of saccharic acid, which comprises
(a) increasing or generating one or more activities selected from the group consisting of DNA binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

A further embodiment of the present invention relates to a process for the production of glucose-1-phosphate, which comprises
(a) increasing or generating one or more activities selected from the group consisting of CCAAT-binding transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of raffinose, which comprises
(a) increasing or generating one or more activities selected from the group consisting of CCAAT-binding transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of fructose, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of sedoheptulose-7-phosphate, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of fructose, which comprises
(a) increasing or generating one or more activities selected from the group consisting of eukaryotic translation initiation factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of eukaryotic translation initiation factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of raffinose, which comprises
(a) increasing or generating one or more activities selected from the group consisting of eukaryotic translation initiation factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of myo-inositol, which comprises
(a) increasing or generating one or more activities selected from the group consisting of mitogen-activated protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sedoheptulose-7-phosphate, which comprises
(a) increasing or generating one or more activities selected from the group consisting of auxin response factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of fructose, which comprises
(a) increasing or generating one or more activities selected from the group consisting of monthiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of monthiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of monthiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of monthiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sedoheptulose-7-phosphate, which comprises
(a) increasing or generating one or more activities selected from the group consisting of monthiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of sucrose, which comprises
(a) increasing or generating one or more activities selected from the group consisting of monthiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sedoheptulose-7-phosphate, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of sucrose, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose-1-phosphate, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of fructose, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sedoheptulose-7-phosphate, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of sucrose, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sedoheptulose-7-phosphate, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of sucrose, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sucrose, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of sedoheptulose-7-phosphate, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of sucrose, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of myo-inositol, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sedoheptulose-7-phosphate, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of sucrose, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose-1-phosphate, which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of raffinose, which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sucrose, which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose-1-phosphate, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of raffinose, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of myo-inositol, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sucrose, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of myo-inositol, which comprises
(a) increasing or generating one or more activities selected from the group consisting of gibberellin 20-oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of myo-inositol, which comprises
(a) increasing or generating one or more activities selected from the group consisting of inositol-3-phosphate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating one or more activities selected from the group consisting of inositol-3-phosphate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of fructose, which comprises
(a) increasing or generating one or more activities selected from the group consisting of At5g16650-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of fructose, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of myo-inositol, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sedoheptulose-7-phosphate, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of sucrose, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose-1-phosphate, which comprises
(a) increasing or generating one or more activities selected from the group consisting of At5g43630-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose-1-phosphate, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating one or more activities selected from the group consisting of RNA-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of RNA-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of fructose, which comprises
(a) increasing or generating one or more activities selected from the group consisting of MADS-box transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of raffinose, which comprises
(a) increasing or generating one or more activities selected from the group consisting of MADS-box transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of saccharic acid, which comprises
(a) increasing or generating one or more activities selected from the group consisting of MADS-box transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

A further embodiment of the present invention relates to a process for the production of glucose-1-phosphate, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of raffinose, which comprises
(a) increasing or generating one or more activities selected from the group consisting of glycosyl transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sucrose, which comprises
(a) increasing or generating one or more activities selected from the group consisting of sec-independent protein translocase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of beta-hydroxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating one or more activities selected from the group consisting of elongation factor Tu in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of elongation factor Tu in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of fructose, which comprises
(a) increasing or generating one or more activities selected from the group consisting of ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of sucrose, which comprises
(a) increasing or generating one or more activities selected from the group consisting of ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose-1-phosphate, which comprises
(a) increasing or generating one or more activities selected from the group consisting of acyl-CoA synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose, which comprises
(a) increasing or generating one or more activities selected from the group consisting of 2-oxoglutarate dehydrogenase E1 subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of 2-oxoglutarate dehydrogenase E1 subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of 2-oxoglutarate dehydrogenase E1 subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating one or more activities selected from the group consisting of 2-oxoglutarate dehydrogenase E1 subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sedoheptulose-7-phosphate, which comprises
(a) increasing or generating one or more activities selected from the group consisting of 2-oxoglutarate dehydrogenase E1 subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of raffinose, which comprises
(a) increasing or generating one or more activities selected from the group consisting of GDP-mannose dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of fructose, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of ThiF family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating one or more activities selected from the group consisting of CTP synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of CTP synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of sucrose, which comprises
(a) increasing or generating one or more activities selected from the group consisting of CTP synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of raffinose, which comprises
(a) increasing or generating one or more activities selected from the group consisting of protein-L-isoaspartate O-methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of myo-inositol, which comprises
(a) increasing or generating one or more activities selected from the group consisting of recombinase A in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating one or more activities selected from the group consisting of recombinase A in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of myo-inositol, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of raffinose, which comprises
(a) increasing or generating one or more activities selected from the group consisting of enoyl-CoA hydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of fructose, which comprises
(a) increasing or generating one or more activities selected from the group consisting of NADH-quinone oxidoreductase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose-1-phosphate, which comprises
(a) increasing or generating one or more activities selected from the group consisting of NADH-quinone oxidoreductase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of sucrose, which comprises
(a) increasing or generating one or more activities selected from the group consisting of NADH-quinone oxidoreductase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of isopropylmalate isomerase large subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating one or more activities selected from the group consisting of hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of sucrose, which comprises
(a) increasing or generating one or more activities selected from the group consisting of hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating one or more activities selected from the group consisting of glutamate-ammonia-ligase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of glutamate-ammonia-ligase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of sucrose, which comprises
(a) increasing or generating one or more activities selected from the group consisting of glutamate-ammonia-ligase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating one or more activities selected from the group consisting of fumarylacetoacetate hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of raffinose, which comprises
(a) increasing or generating one or more activities selected from the group consisting of glycosyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of myo-inositol, which comprises
(a) increasing or generating one or more activities selected from the group consisting of peptidyl-prolyl cis-trans isomerase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of NAD(P)H dehydrogenase (quinone) in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating one or more activities selected from the group consisting of electron transfer flavoprotein subunit beta in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of electron transfer flavoprotein subunit beta in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of purine nucleoside phosphorylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of purine nucleoside phosphorylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of raffinose, which comprises
(a) increasing or generating one or more activities selected from the group consisting of transcriptional regulator protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose-1-phosphate, which comprises
(a) increasing or generating one or more activities selected from the group consisting of AX653549-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating one or more activities selected from the group consisting of AY087308-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of AY087308-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of glucose-1-phosphate, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose, which comprises
(a) increasing or generating one or more activities selected from the group consisting of threonine synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of sucrose, which comprises
(a) increasing or generating one or more activities selected from the group consisting of threonine synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of b0050-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of raffinose, which comprises
(a) increasing or generating one or more activities selected from the group consisting of L-ribulose-5-phosphate 4-epimerase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sucrose, which comprises
(a) increasing or generating one or more activities selected from the group consisting of glucose dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose-1-phosphate, which comprises
(a) increasing or generating one or more activities selected from the group consisting of serine protease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of serine protease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sucrose, which comprises
(a) increasing or generating one or more activities selected from the group consisting of acyl-CoA dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose-1-phosphate, which comprises
(a) increasing or generating one or more activities selected from the group consisting of b0251-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of fructose, which comprises
(a) increasing or generating one or more activities selected from the group consisting of betaine aldehyde dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of melibiose, which comprises
(a) increasing or generating one or more activities selected from the group consisting of beta-galactosidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of melibiose or a composition comprising melibiose in said non-human organism or in the culture medium surrounding said non-human organism.

A further embodiment of the present invention relates to a process for the production of glucose-1-phosphate, which comprises
(a) increasing or generating one or more activities selected from the group consisting of b0362-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating one or more activities selected from the group consisting of penicillin-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of fructose, which comprises
(a) increasing or generating one or more activities selected from the group consisting of ATP-binding component of a transport system in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of ATP-binding component of a transport system in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose, which comprises
(a) increasing or generating one or more activities selected from the group consisting of membrane transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of saccharic acid, which comprises
(a) increasing or generating one or more activities selected from the group consisting of b0488-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

A further embodiment of the present invention relates to a process for the production of galactinol, which comprises
(a) increasing or generating one or more activities selected from the group consisting of glycerate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

A further embodiment of the present invention relates to a process for the production of fructose, which comprises
(a) increasing or generating one or more activities selected from the group consisting of isochorismate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of isochorismate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of raffinose, which comprises
(a) increasing or generating one or more activities selected from the group consisting of isochorismate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sucrose, which comprises
(a) increasing or generating one or more activities selected from the group consisting of isochorismate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of B0644-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of saccharic acid, which comprises
(a) increasing or generating one or more activities selected from the group consisting of pyrimidine-specific ribonucleoside hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

A further embodiment of the present invention relates to a process for the production of erythrol, which comprises
(a) increasing or generating one or more activities selected from the group consisting of asparagine synthetase B in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of erythrol or a composition comprising erythrol in said non-human organism or in the culture medium surrounding said non-human organism.

A further embodiment of the present invention relates to a process for the production of myo-inositol, which comprises
(a) increasing or generating one or more activities selected from the group consisting of sensor histidine kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of myo-inositol, which comprises
(a) increasing or generating one or more activities selected from the group consisting of succinyl-CoA synthetase beta chain in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating one or more activities selected from the group consisting of zinc transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of zinc transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of zinc transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of raffinose, which comprises
(a) increasing or generating one or more activities selected from the group consisting of zinc transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose, which comprises
(a) increasing or generating one or more activities selected from the group consisting of b0801-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of sedoheptulose-7-phosphate, which comprises
(a) increasing or generating one or more activities selected from the group consisting of putrescine transport system permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of raffinose, which comprises
(a) increasing or generating one or more activities selected from the group consisting of leucyl/phenylalanyl-tRNA-protein transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose-1-phosphate, which comprises
(a) increasing or generating one or more activities selected from the group consisting of b0970-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of phosphoanhydride phosphorylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating one or more activities selected from the group consisting of b1003-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of b1003-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of b1003-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of sedoheptulose-7-phosphate, which comprises
(a) increasing or generating one or more activities selected from the group consisting of b1003-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of sucrose, which comprises
(a) increasing or generating one or more activities selected from the group consisting of b1003-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of b1046-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating one or more activities selected from the group consisting of multidrug resistance protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of sucrose, which comprises
(a) increasing or generating one or more activities selected from the group consisting of multidrug resistance protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating one or more activities selected from the group consisting of outer membrane receptor protein precursor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose-1-phosphate, which comprises
(a) increasing or generating one or more activities selected from the group consisting of adenylosuccinate lyase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose, which comprises
(a) increasing or generating one or more activities selected from the group consisting of isocitrate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of b1137-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating one or more activities selected from the group consisting of sodium/proton antiporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating one or more activities selected from the group consisting of b1214-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of b1214-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating one or more activities selected from the group consisting of b1219-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of b1219-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of b1219-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of myo-inositol, which comprises
(a) increasing or generating one or more activities selected from the group consisting of b1234-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of galactinol, which comprises
(a) increasing or generating one or more activities selected from the group consisting of cardiolipin synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

A further embodiment of the present invention relates to a process for the production of galactitol, which comprises
(a) increasing or generating one or more activities selected from the group consisting of cardiolipin synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactitol or a composition comprising galactitol in said non-human organism or in the culture medium surrounding said non-human organism.

A further embodiment of the present invention relates to a process for the production of sorbitol, which comprises
(a) increasing or generating one or more activities selected from the group consisting of cardiolipin synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sorbitol or a composition comprising sorbitol in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of intracellular septation protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating one or more activities selected from the group consisting of membrane protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of membrane protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of raffinose, which comprises
(a) increasing or generating one or more activities selected from the group consisting of membrane protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sucrose, which comprises
(a) increasing or generating one or more activities selected from the group consisting of membrane protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of myo-inositol, which comprises
(a) increasing or generating one or more activities selected from the group consisting of tryptophan biosynthesis protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating one or more activities selected from the group consisting of b1285-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose-1-phosphate, which comprises
(a) increasing or generating one or more activities selected from the group consisting of phage-related repressor protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating one or more activities selected from the group consisting of b1357-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose, which comprises
(a) increasing or generating one or more activities selected from the group consisting of phenylacetic acid degradation protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of phenylacetic acid degradation protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of sedoheptulose-7-phosphate, which comprises
(a) increasing or generating one or more activities selected from the group consisting of b1410-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of myo-inositol, which comprises
(a) increasing or generating one or more activities selected from the group consisting of b1425-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of glucose-1-phosphate, which comprises
(a) increasing or generating one or more activities selected from the group consisting of lipoprotein precursor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of sedoheptulose-7-phosphate, which comprises
(a) increasing or generating one or more activities selected from the group consisting of lipoprotein precursor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of lipoprotein precursor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of b1445-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating one or more activities selected from the group consisting of b1470-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of fructose, which comprises
(a) increasing or generating one or more activities selected from the group consisting of b1522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose-1-phosphate, which comprises
(a) increasing or generating one or more activities selected from the group consisting of b1522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating one or more activities selected from the group consisting of b1556-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of sedoheptulose-7-phosphate, which comprises
(a) increasing or generating one or more activities selected from the group consisting of b1556-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of glucose-1-phosphate, which comprises
(a) increasing or generating one or more activities selected from the group consisting of b1583-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating one or more activities selected from the group consisting of Fe—S subunit of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of Fe—S subunit of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating one or more activities selected from the group consisting of acid shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of acid shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of acid shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of acid shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating one or more activities selected from the group consisting of acid shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of fructose, which comprises
(a) increasing or generating one or more activities selected from the group consisting of phosphotransferase system component in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of phosphotransferase system component in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose, which comprises
(a) increasing or generating one or more activities selected from the group consisting of electron transport complex protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of electron transport complex protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of electron transport complex protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of myo-inositol, which comprises
(a) increasing or generating one or more activities selected from the group consisting of methylglyoxalase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of myo-inositol, which comprises
(a) increasing or generating one or more activities selected from the group consisting of b1670-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating one or more activities selected from the group consisting of ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of myo-inositol, which comprises
(a) increasing or generating one or more activities selected from the group consisting of transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating one or more activities selected from the group consisting of b1796-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of myo-inositol, which comprises
(a) increasing or generating one or more activities selected from the group consisting of para-aminobenzoate synthase component I in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of galactitol, which comprises
(a) increasing or generating one or more activities selected from the group consisting of L-serine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactitol or a composition comprising galactitol in said non-human organism or in the culture medium surrounding said non-human organism.

A further embodiment of the present invention relates to a process for the production of sorbitol, which comprises
(a) increasing or generating one or more activities selected from the group consisting of L-serine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sorbitol or a composition comprising sorbitol in said non-human organism or in the culture medium surrounding said non-human organism.

A further embodiment of the present invention relates to a process for the production of myo-inositol, which comprises
(a) increasing or generating one or more activities selected from the group consisting of protease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of erythrol, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of erythrol or a composition comprising erythrol in said non-human organism or in the culture medium surrounding said non-human organism.

A further embodiment of the present invention relates to a process for the production of myo-inositol, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sorbitol, which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sorbitol or a composition comprising sorbitol in said non-human organism or in the culture medium surrounding said non-human organism.

A further embodiment of the present invention relates to a process for the production of fructose, which comprises
(a) increasing or generating one or more activities selected from the group consisting of dATP pyrophosphohydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose-1-phosphate, which comprises
(a) increasing or generating one or more activities selected from the group consisting of trehalose-6-phosphate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of trehalose, which comprises
(a) increasing or generating one or more activities selected from the group consisting of trehalose-6-phosphate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of trehalose or a composition comprising trehalose in said non-human organism or in the culture medium surrounding said non-human organism.

A further embodiment of the present invention relates to a process for the production of erythrol, which comprises
(a) increasing or generating one or more activities selected from the group consisting of trehalose-phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of erythrol or a composition comprising erythrol in said non-human organism or in the culture medium surrounding said non-human organism.

A further embodiment of the present invention relates to a process for the production of fructose, which comprises
(a) increasing or generating one or more activities selected from the group consisting of trehalose-phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of trehalose-phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of sucrose, which comprises
(a) increasing or generating one or more activities selected from the group consisting of trehalose-phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of raffinose, which comprises
(a) increasing or generating one or more activities selected from the group consisting of b1898-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose-1-phosphate, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose, which comprises
(a) increasing or generating one or more activities selected from the group consisting of flagellar M-ring protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of myo-inositol, which comprises
(a) increasing or generating one or more activities selected from the group consisting of flagellar M-ring protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating one or more activities selected from the group consisting of O-antigen chain length determinant in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of b2032-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of myo-inositol, which comprises
(a) increasing or generating one or more activities selected from the group consisting of b2032-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of glucose-1-phosphate, which comprises
(a) increasing or generating one or more activities selected from the group consisting of colanic acid biosynthesis protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of fucose, which comprises
(a) increasing or generating one or more activities selected from the group consisting of GDP-mannose 4,6-dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fucose or a composition comprising fucose in said non-human organism or in the culture medium surrounding said non-human organism.

A further embodiment of the present invention relates to a process for the production of fructose, which comprises
(a) increasing or generating one or more activities selected from the group consisting of transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose-1-phosphate, which comprises
(a) increasing or generating one or more activities selected from the group consisting of transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of raffinose, which comprises
(a) increasing or generating one or more activities selected from the group consisting of transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sucrose, which comprises
(a) increasing or generating one or more activities selected from the group consisting of transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating one or more activities selected from the group consisting of b2107-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of b2107-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating one or more activities selected from the group consisting of b2139-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of erythrol, which comprises
(a) increasing or generating one or more activities selected from the group consisting of sn-glycerol-3-phosphate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of erythrol or a composition comprising erythrol in said non-human organism or in the culture medium surrounding said non-human organism.

A further embodiment of the present invention relates to a process for the production of fructose, which comprises
(a) increasing or generating one or more activities selected from the group consisting of ATP synthase subunit E in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of galactinol, which comprises
(a) increasing or generating one or more activities selected from the group consisting of aminotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

A further embodiment of the present invention relates to a process for the production of myo-inositol, which comprises
(a) increasing or generating one or more activities selected from the group consisting of b2345-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of b2345-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of verbascose, which comprises
(a) increasing or generating one or more activities selected from the group consisting of b2360-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of verbascose or a composition comprising verbascose in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of b2360-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating one or more activities selected from the group consisting of b2361-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of b2361-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of sucrose, which comprises
(a) increasing or generating one or more activities selected from the group consisting of ion-transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating one or more activities selected from the group consisting of b2399-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of b2399-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of b2399-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of b2399-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating one or more activities selected from the group consisting of b2399-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sedoheptulose-7-phosphate, which comprises
(a) increasing or generating one or more activities selected from the group consisting of b2399-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of sucrose, which comprises
(a) increasing or generating one or more activities selected from the group consisting of b2399-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of raffinose, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of myo-inositol, which comprises
(a) increasing or generating one or more activities selected from the group consisting of cysteine synthase A in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of verbascose, which comprises
(a) increasing or generating one or more activities selected from the group consisting of cysteine synthase A in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of verbascose or a composition comprising verbascose in said non-human organism or in the culture medium surrounding said non-human organism.

A further embodiment of the present invention relates to a process for the production of glucose-1-phosphate, which comprises
(a) increasing or generating one or more activities selected from the group consisting of b2466-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of b2466-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of b2466-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating one or more activities selected from the group consisting of b2474-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of raffinose, which comprises
(a) increasing or generating one or more activities selected from the group consisting of 2,3-dihydroxy-2,3-dihydrophenylpropionatedehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sucrose, which comprises
(a) increasing or generating one or more activities selected from the group consisting of putative transport system permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose-1-phosphate, which comprises
(a) increasing or generating one or more activities selected from the group consisting of b2613-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of b2613-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of myo-inositol, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sucrose, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose-1-phosphate, which comprises
(a) increasing or generating one or more activities selected from the group consisting of b2653-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of sedoheptulose-7-phosphate, which comprises
(a) increasing or generating one or more activities selected from the group consisting of b2653-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of b2653-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating one or more activities selected from the group consisting of b2654-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of b2654-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating one or more activities selected from the group consisting of b2670-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating one or more activities selected from the group consisting of b2673-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of b2673-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of myo-inositol, which comprises
(a) increasing or generating one or more activities selected from the group consisting of b2682-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of glucose-1-phosphate, which comprises
(a) increasing or generating one or more activities selected from the group consisting of recombinase A in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating one or more activities selected from the group consisting of murein transglycosylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating one or more activities selected from the group consisting of b2739-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of b2739-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose, which comprises
(a) increasing or generating one or more activities selected from the group consisting of 2-C-methyl-D-erythritol 4-phosphate cytidylyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of myo-inositol, which comprises
(a) increasing or generating one or more activities selected from the group consisting of 2-C-methyl-D-erythritol 4-phosphate cytidylyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating one or more activities selected from the group consisting of 2-C-methyl-D-erythritol 4-phosphate cytidylyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose-1-phosphate, which comprises
(a) increasing or generating one or more activities selected from the group consisting of phosphoadenosine phosphosulfate reductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of phosphoadenosine phosphosulfate reductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating one or more activities selected from the group consisting of flavodoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of flavodoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of flavodoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of flavodoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of flavodoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating one or more activities selected from the group consisting of amino-acid acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose-1-phosphate, which comprises
(a) increasing or generating one or more activities selected from the group consisting of amino-acid acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of amino-acid acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose, which comprises
(a) increasing or generating one or more activities selected from the group consisting of arginine exporter protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of myo-inositol, which comprises
(a) increasing or generating one or more activities selected from the group consisting of arginine exporter protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of myo-inositol, which comprises
(a) increasing or generating one or more activities selected from the group consisting of b2932-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of erythrol, which comprises
(a) increasing or generating one or more activities selected from the group consisting of L-serine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of erythrol or a composition comprising erythrol in said non-human organism or in the culture medium surrounding said non-human organism.

A further embodiment of the present invention relates to a process for the production of myo-inositol, which comprises
(a) increasing or generating one or more activities selected from the group consisting of b3151-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating one or more activities selected from the group consisting of b3151-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sucrose, which comprises
(a) increasing or generating one or more activities selected from the group consisting of b3151-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating one or more activities selected from the group consisting of b3221-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of b3221-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of b3221-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of myo-inositol, which comprises
(a) increasing or generating one or more activities selected from the group consisting of b3246-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of myo-inositol, which comprises
(a) increasing or generating one or more activities selected from the group consisting of acetyl CoA carboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating one or more activities selected from the group consisting of methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of raffinose, which comprises
(a) increasing or generating one or more activities selected from the group consisting of methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sucrose, which comprises
(a) increasing or generating one or more activities selected from the group consisting of methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose, which comprises
(a) increasing or generating one or more activities selected from the group consisting of ferredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of myo-inositol, which comprises
(a) increasing or generating one or more activities selected from the group consisting of b3346-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating one or more activities selected from the group consisting of b3346-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of b3346-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating one or more activities selected from the group consisting of phosphoenolpyruvate carboxykinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of phosphoenolpyruvate carboxykinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of B3427-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating one or more activities selected from the group consisting of glycogen synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of glycogen synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of myo-inositol, which comprises
(a) increasing or generating one or more activities selected from the group consisting of b3442-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of B3443-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of saccharic acid, which comprises
(a) increasing or generating one or more activities selected from the group consisting of sn-glycerol-3-phosphate transport system permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

A further embodiment of the present invention relates to a process for the production of glucose-1-phosphate, which comprises
(a) increasing or generating one or more activities selected from the group consisting of high-affinity branched-chain amino acid transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of high-affinity branched-chain amino acid transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating one or more activities selected from the group consisting of trehalase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of galactinol, which comprises
(a) increasing or generating one or more activities selected from the group consisting of trehalase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

A further embodiment of the present invention relates to a process for the production of fructose, which comprises
(a) increasing or generating one or more activities selected from the group consisting of mannitol-1-phosphate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of mannitol, which comprises
(a) increasing or generating one or more activities selected from the group consisting of mannitol-1-phosphate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of mannitol or a composition comprising mannitol in said non-human organism or in the culture medium surrounding said non-human organism.

A further embodiment of the present invention relates to a process for the production of sorbitol, which comprises
(a) increasing or generating one or more activities selected from the group consisting of mannitol-1-phosphate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sorbitol or a composition comprising sorbitol in said non-human organism or in the culture medium surrounding said non-human organism.

A further embodiment of the present invention relates to a process for the production of galactinol, which comprises
(a) increasing or generating one or more activities selected from the group consisting of ketol-acid reductoisomerase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

A further embodiment of the present invention relates to a process for the production of myo-inositol, which comprises
(a) increasing or generating one or more activities selected from the group consisting of b3777-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of myo-inositol, which comprises
(a) increasing or generating one or more activities selected from the group consisting of b3817-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating one or more activities selected from the group consisting of b3817-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of myo-inositol, which comprises
(a) increasing or generating one or more activities selected from the group consisting of b3818-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of myo-inositol, which comprises
(a) increasing or generating one or more activities selected from the group consisting of threonine efflux protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of myo-inositol, which comprises
(a) increasing or generating one or more activities selected from the group consisting of glutamine synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of verbascose, which comprises
(a) increasing or generating one or more activities selected from the group consisting of glutamine synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of verbascose or a composition comprising verbascose in said non-human organism or in the culture medium surrounding said non-human organism.

A further embodiment of the present invention relates to a process for the production of glucose-1-phosphate, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of fructose, which comprises
(a) increasing or generating one or more activities selected from the group consisting of b3989-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of b3989-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of b3989-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of raffinose, which comprises
(a) increasing or generating one or more activities selected from the group consisting of b3989-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sucrose, which comprises
(a) increasing or generating one or more activities selected from the group consisting of b3989-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of galactinol, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

A further embodiment of the present invention relates to a process for the production of fructose, which comprises
(a) increasing or generating one or more activities selected from the group consisting of b4029-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of b4029-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of sedoheptulose-7-phosphate, which comprises
(a) increasing or generating one or more activities selected from the group consisting of b4029-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of glucose-1-phosphate, which comprises
(a) increasing or generating one or more activities selected from the group consisting of chorismate lyase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of chorismate lyase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating one or more activities selected from the group consisting of b4050-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of b4050-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of myo-inositol, which comprises
(a) increasing or generating one or more activities selected from the group consisting of b4050-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating one or more activities selected from the group consisting of b4056-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of galactinol, which comprises
(a) increasing or generating one or more activities selected from the group consisting of acetyl-coenzyme A synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

A further embodiment of the present invention relates to a process for the production of glucose-1-phosphate, which comprises
(a) increasing or generating one or more activities selected from the group consisting of lipoprotein precursor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of lipoprotein precursor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of myo-inositol, which comprises
(a) increasing or generating one or more activities selected from the group consisting of b4250-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating one or more activities selected from the group consisting of b4250-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of fructose, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of fructose, which comprises
(a) increasing or generating one or more activities selected from the group consisting of gluconate transport system permease 3 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of galactinol, which comprises
(a) increasing or generating one or more activities selected from the group consisting of purine-nucleoside phosphorylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

A further embodiment of the present invention relates to a process for the production of myo-inositol, which comprises
(a) increasing or generating one or more activities selected from the group consisting of lysine decarboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sucrose, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose-1-phosphate, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose-1-phosphate, which comprises
(a) increasing or generating one or more activities selected from the group consisting of GM02LC38418-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of raffinose, which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sucrose, which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of GM02LC46-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of myo-inositol, which comprises
(a) increasing or generating one or more activities selected from the group consisting of alkaline phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of verbascose, which comprises
(a) increasing or generating one or more activities selected from the group consisting of flavodoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of verbascose or a composition comprising verbascose in said non-human organism or in the culture medium surrounding said non-human organism.

A further embodiment of the present invention relates to a process for the production of sucrose, which comprises
(a) increasing or generating one or more activities selected from the group consisting of sll0254-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of verbascose, which comprises
(a) increasing or generating one or more activities selected from the group consisting of sll0281-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of verbascose or a composition comprising verbascose in said non-human organism or in the culture medium surrounding said non-human organism.

A further embodiment of the present invention relates to a process for the production of myo-inositol, which comprises
(a) increasing or generating one or more activities selected from the group consisting of sll0354-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of myo-inositol, which comprises
(a) increasing or generating one or more activities selected from the group consisting of bifunctional protein (phosphoribosyltransferase and regulatory protein) in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of glucose-1-phosphate, which comprises
(a) increasing or generating one or more activities selected from the group consisting of sll0418-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of sll0418-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of myo-inositol, which comprises
(a) increasing or generating one or more activities selected from the group consisting of sll0418-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of saccharic acid, which comprises
(a) increasing or generating one or more activities selected from the group consisting of sll0418-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of sll0418-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating one or more activities selected from the group consisting of urease subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating one or more activities selected from the group consisting of glucokinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of saccharic acid, which comprises
(a) increasing or generating one or more activities selected from the group consisting of urease accessory protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

A further embodiment of the present invention relates to a process for the production of glucose-1-phosphate, which comprises
(a) increasing or generating one or more activities selected from the group consisting of binding-protein-dependent transport systems inner membrane component in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of binding-protein-dependent transport systems inner membrane component in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating one or more activities selected from the group consisting of 3-deoxy-7-phosphoheptulonate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of fructose, which comprises
(a) increasing or generating one or more activities selected from the group consisting of glycogen synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of sucrose, which comprises
(a) increasing or generating one or more activities selected from the group consisting of glycogen synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose-1-phosphate, which comprises
(a) increasing or generating one or more activities selected from the group consisting of coproporphyrinogen oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of short chain dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of raffinose, which comprises
(a) increasing or generating one or more activities selected from the group consisting of glycogen (starch) synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of myo-inositol, which comprises
(a) increasing or generating one or more activities selected from the group consisting of carbamoyl-phosphate synthase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of carbamoyl-phosphate synthase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of myo-inositol, which comprises
(a) increasing or generating one or more activities selected from the group consisting of 4-alpha-glucanotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of alanine dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose, which comprises
(a) increasing or generating one or more activities selected from the group consisting of sll1761-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose, which comprises
(a) increasing or generating one or more activities selected from the group consisting of acyl transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of cation-transporting ATPase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating one or more activities selected from the group consisting of cation-transporting ATPase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of cation-transporting ATPase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating one or more activities selected from the group consisting of cation-transporting ATPase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose-1-phosphate, which comprises
(a) increasing or generating one or more activities selected from the group consisting of glycosidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of fructose, which comprises
(a) increasing or generating one or more activities selected from the group consisting of precorrin methylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of precorrin methylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating one or more activities selected from the group consisting of slr0600-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating one or more activities selected from the group consisting of Glu/Leu/Phe/Val dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating one or more activities selected from the group consisting of malate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of sucrose, which comprises
(a) increasing or generating one or more activities selected from the group consisting of geranylgeranyl pyrophosphate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating one or more activities selected from the group consisting of circadian clock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of circadian clock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of circadian clock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating one or more activities selected from the group consisting of amine oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of fructose, which comprises
(a) increasing or generating one or more activities selected from the group consisting of kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of Mg-protoporphyrin IX monomethyl ester oxidative cyclase 66 kD subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of permease protein of ABC transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of glucose-1-phosphate, which comprises
(a) increasing or generating one or more activities selected from the group consisting of dihydrolipoamide dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of raffinose, which comprises
(a) increasing or generating one or more activities selected from the group consisting of dihydrolipoamide dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of myo-inositol, which comprises
(a) increasing or generating one or more activities selected from the group consisting of slr1107-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of glucose-1-phosphate, which comprises
(a) increasing or generating one or more activities selected from the group consisting of fatty acid desaturase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating one or more activities selected from the group consisting of CDP-diglyceride synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of CDP-diglyceride synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating one or more activities selected from the group consisting of iron(III) dicitrate-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of sucrose, which comprises
(a) increasing or generating one or more activities selected from the group consisting of photosystem I reaction center subunit XI in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating one or more activities selected from the group consisting of photosystem II protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of fructose, which comprises
(a) increasing or generating one or more activities selected from the group consisting of glutamine amidotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of myo-inositol, which comprises
(a) increasing or generating one or more activities selected from the group consisting of glutamine amidotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of glucose-1-phosphate, which comprises
(a) increasing or generating one or more activities selected from the group consisting of NADH dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of raffinose, which comprises
(a) increasing or generating one or more activities selected from the group consisting of pyridoxal phosphate biosynthetic protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose-1-phosphate, which comprises
(a) increasing or generating one or more activities selected from the group consisting of phosphoadenosine phosphosulfate reductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating one or more activities selected from the group consisting of threonine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of threonine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating one or more activities selected from the group consisting of short-chain alcohol dehydrogenase family in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of short-chain alcohol dehydrogenase family in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of short-chain alcohol dehydrogenase family in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of short-chain alcohol dehydrogenase family in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sucrose, which comprises
(a) increasing or generating one or more activities selected from the group consisting of short-chain alcohol dehydrogenase family in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of raffinose, which comprises
(a) increasing or generating one or more activities selected from the group consisting of phosphate acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose-1-phosphate, which comprises
(a) increasing or generating one or more activities selected from the group consisting of photosystem I reaction centre subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of raffinose, which comprises
(a) increasing or generating one or more activities selected from the group consisting of photosystem I reaction centre subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of photosystem I reaction centre subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating one or more activities selected from the group consisting of Sec-independent protein translocase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of Sec-independent protein translocase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of sedoheptulose-7-phosphate, which comprises
(a) increasing or generating one or more activities selected from the group consisting of Sec-independent protein translocase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of fructose, which comprises
(a) increasing or generating one or more activities selected from the group consisting of cell division protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of cell division protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of sucrose, which comprises
(a) increasing or generating one or more activities selected from the group consisting of cell division protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose-1-phosphate, which comprises
(a) increasing or generating one or more activities selected from the group consisting of branched-chain amino acid ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of branched-chain amino acid ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of myo-inositol, which comprises
(a) increasing or generating one or more activities selected from the group consisting of 3-isopropylmalate dehydratase large subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating one or more activities selected from the group consisting of multiple antibiotic resistance protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of multiple antibiotic resistance protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of multiple antibiotic resistance protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of fructose, which comprises
(a) increasing or generating one or more activities selected from the group consisting of TTC1386-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of TTC1386-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of fructose, which comprises
(a) increasing or generating one or more activities selected from the group consisting of homocitrate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose-1-phosphate, which comprises
(a) increasing or generating one or more activities selected from the group consisting of homocitrate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of sucrose, which comprises
(a) increasing or generating one or more activities selected from the group consisting of homocitrate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of raffinose, which comprises
(a) increasing or generating one or more activities selected from the group consisting of permease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of permease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating one or more activities selected from the group consisting of oxidoreductase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating one or more activities selected from the group consisting of XM_{—}473199-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sucrose, which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose-1-phosphate, which comprises
(a) increasing or generating one or more activities selected from the group consisting of 3-deoxy-7-phosphoheptulonate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating one or more activities selected from the group consisting of phosphate permease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of phosphate permease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating one or more activities selected from the group consisting of ycl027w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose-1-phosphate, which comprises
(a) increasing or generating one or more activities selected from the group consisting of ycl027w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of serine/threonine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sucrose, which comprises
(a) increasing or generating one or more activities selected from the group consisting of serine/threonine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of fructose, which comprises
(a) increasing or generating one or more activities selected from the group consisting of phosphoglycerate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of phosphoglycerate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of raffinose, which comprises
(a) increasing or generating one or more activities selected from the group consisting of YCR047W-A-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of ycr102c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of galactinol, which comprises
(a) increasing or generating one or more activities selected from the group consisting of isocitrate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

A further embodiment of the present invention relates to a process for the production of myo-inositol, which comprises
(a) increasing or generating one or more activities selected from the group consisting of cell division control protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of myo-inositol, which comprises
(a) increasing or generating one or more activities selected from the group consisting of cyclin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating one or more activities selected from the group consisting of YDL159W-A-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose-1-phosphate, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating one or more activities selected from the group consisting of serine/threonine-protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of integral membrane protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating one or more activities selected from the group consisting of ydl235c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of ydl235c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of ydl235c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of sucrose, which comprises
(a) increasing or generating one or more activities selected from the group consisting of coproporphyrinogen III oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose, which comprises
(a) increasing or generating one or more activities selected from the group consisting of branched-chain amino acid permease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of fructose, which comprises
(a) increasing or generating one or more activities selected from the group consisting of ydr183w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of ydr183w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of ydr507c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of fructose, which comprises
(a) increasing or generating one or more activities selected from the group consisting of 1,4-alpha-glucan branching enzyme in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of 1,4-alpha-glucan branching enzyme in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of YEL030C-A-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating one or more activities selected from the group consisting of aldolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose, which comprises
(a) increasing or generating one or more activities selected from the group consisting of O-acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of raffinose, which comprises
(a) increasing or generating one or more activities selected from the group consisting of yer177w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of myo-inositol, which comprises
(a) increasing or generating one or more activities selected from the group consisting of YFL019C-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sucrose, which comprises
(a) increasing or generating one or more activities selected from the group consisting of YFL019C-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of sucrose, which comprises
(a) increasing or generating one or more activities selected from the group consisting of hexokinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose-1-phosphate, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating one or more activities selected from the group consisting of ygl096w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of ygl096w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of raffinose, which comprises
(a) increasing or generating one or more activities selected from the group consisting of ygl237c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of glucose-1-phosphate, which comprises
(a) increasing or generating one or more activities selected from the group consisting of alcohol dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of alcohol dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose, which comprises
(a) increasing or generating one or more activities selected from the group consisting of yhl013c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of yhl013c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of glucose-1-phosphate, which comprises
(a) increasing or generating one or more activities selected from the group consisting of 3-phosphoglycerate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of fructose, which comprises
(a) increasing or generating one or more activities selected from the group consisting of YIL083C-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of myo-inositol, which comprises
(a) increasing or generating one or more activities selected from the group consisting of DnaJ-like chaperone in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of YJL127W-A-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating one or more activities selected from the group consisting of inositol-3-phosphate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of galactitol, which comprises
(a) increasing or generating one or more activities selected from the group consisting of inositol-3-phosphate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactitol or a composition comprising galactitol in said non-human organism or in the culture medium surrounding said non-human organism.

A further embodiment of the present invention relates to a process for the production of myo-inositol, which comprises
(a) increasing or generating one or more activities selected from the group consisting of inositol-3-phosphate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sorbitol, which comprises
(a) increasing or generating one or more activities selected from the group consisting of hexose transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sorbitol or a composition comprising sorbitol in said non-human organism or in the culture medium surrounding said non-human organism.

A further embodiment of the present invention relates to a process for the production of myo-inositol, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of glucose, which comprises
(a) increasing or generating one or more activities selected from the group consisting of cystathionine gamma-synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of fructose, which comprises
(a) increasing or generating one or more activities selected from the group consisting of fructose biphosphate aldolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of sedoheptulose-7-phosphate, which comprises
(a) increasing or generating one or more activities selected from the group consisting of fructose biphosphate aldolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of glucose-1-phosphate, which comprises
(a) increasing or generating one or more activities selected from the group consisting of acyl carrier protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of sucrose, which comprises
(a) increasing or generating one or more activities selected from the group consisting of halotolerance protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose, which comprises
(a) increasing or generating one or more activities selected from the group consisting of phosphoenolpyruvate carboxykinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose, which comprises
(a) increasing or generating one or more activities selected from the group consisting of phosphoenolpyruvate carboxykinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of sucrose, which comprises
(a) increasing or generating one or more activities selected from the group consisting of vacuolar sorting protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of glucose-1-phosphate, which comprises
(a) increasing or generating one or more activities selected from the group consisting of thioredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of fructose, which comprises
(a) increasing or generating one or more activities selected from the group consisting of hexose transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of verbascose, which comprises
(a) increasing or generating one or more activities selected from the group consisting of purine biosynthesis protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of verbascose or a composition comprising verbascose in said non-human organism or in the culture medium surrounding said non-human organism.

A further embodiment of the present invention relates to a process for the production of glucose-1-phosphate, which comprises
(a) increasing or generating one or more activities selected from the group consisting of ymr324c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of molecular chaperone in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sucrose, which comprises
(a) increasing or generating one or more activities selected from the group consisting of molecular chaperone in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of raffinose, which comprises
(a) increasing or generating one or more activities selected from the group consisting of sterol O-acyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of myo-inositol, which comprises
(a) increasing or generating one or more activities selected from the group consisting of yol160w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of glucose, which comprises
(a) increasing or generating one or more activities selected from the group consisting of phosphohydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of histone acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of subunit of COMPASS-protein complex in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating one or more activities selected from the group consisting of ypl144w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of ypl144w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating one or more activities selected from the group consisting of ypr098c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of ypr098c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of glucose-1-phosphate, which comprises
(a) increasing or generating one or more activities selected from the group consisting of polyamine transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of polyamine transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating one or more activities selected from the group consisting of Zm_{—}4842_BE510522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of Zm_{—}4842_BE510522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of myo-inositol, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of fructose, which comprises
(a) increasing or generating one or more activities selected from the group consisting of 47266012-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of 47266012-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of 47266012-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of 47266012-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of sedoheptulose-7-phosphate, which comprises
(a) increasing or generating one or more activities selected from the group consisting of 47266012-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of glucose, which comprises
(a) increasing or generating one or more activities selected from the group consisting of 49747384_SOYBEAN-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of 49747384_SOYBEAN-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of myo-inositol, which comprises
(a) increasing or generating one or more activities selected from the group consisting of 49747384_SOYBEAN-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

A further embodiment of the present invention relates to a process for the production of raffinose, which comprises
(a) increasing or generating one or more activities selected from the group consisting of 49747384_SOYBEAN-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

A further embodiment of the present invention relates to a process for the production of sedoheptulose-7-phosphate, which comprises
(a) increasing or generating one or more activities selected from the group consisting of 49747384_SOYBEAN-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of fructose, which comprises
(a) increasing or generating one or more activities selected from the group consisting of glucose-6-phosphate 1-dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of glucose-6-phosphate 1-dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of glucose-1-phosphate, which comprises
(a) increasing or generating one or more activities selected from the group consisting of glucose-6-phosphate 1-dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

A further embodiment of the present invention relates to a process for the production of sedoheptulose-7-phosphate, which comprises
(a) increasing or generating one or more activities selected from the group consisting of glucose-6-phosphate 1-dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

A further embodiment of the present invention relates to a process for the production of sucrose, which comprises
(a) increasing or generating one or more activities selected from the group consisting of glucose-6-phosphate 1-dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

A further embodiment of the present invention relates to a process for the production of 3,4,5-trihydroxypentanoic acid, which comprises
(a) increasing or generating one or more activities selected from the group consisting of glucose-6-phosphate 1-dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

A further embodiment of the present invention relates to a process for the production of fructose, which comprises
(a) increasing or generating one or more activities selected from the group consisting of DNA replication complex GINS protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

A further embodiment of the present invention relates to a process for the production of glucose, which comprises
(a) increasing or generating one or more activities selected from the group consisting of acetyl CoA synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

A further embodiment of the present invention relates to a process for the production of 3,4,5-trihydroxypentanoic acid, which comprises
(a1) increasing or generating the activity of a polypeptide comprising a polypeptide as depicted in the respective line in column 5 or 8 of Table II, application no. 17, or a homolog or fragment thereof, a consensus sequence or at least one polypeptide motif as depicted in column 8 of Table IV, application no. 17, whereby the respective line disclose in column 7 the fine chemical 3,4,5-trihydroxypentanoic acid; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical 3,4,5-trihydroxypentanoic acid;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, which is joined to a transit peptide, whereby the respective line disclose in column 7 the fine chemical 3,4,5-trihydroxypentanoic acid; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical 3,4,5-trihydroxypentanoic acid;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical 3,4,5-trihydroxypentanoic acid; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical 3,4,5-trihydroxypentanoic acid;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of 3,4,5-trihydroxypentanoic acid, or a composition comprising 3,4,5-trihydroxypentanoic acid in said non-human organism or in the culture medium surrounding said non-human organism.

A further embodiment of the present invention relates to a process for the production of erythrol, which comprises
(a1) increasing or generating the activity of a polypeptide comprising a polypeptide as depicted in the respective line in column 5 or 8 of Table II, application no. 17, or a homolog or fragment thereof, a consensus sequence or at least one polypeptide motif as depicted in column 8 of Table IV, application no. 17, whereby the respective line disclose in column 7 the fine chemical erythrol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical erythrol;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, which is joined to a transit peptide, whereby the respective line disclose in column 7 the fine chemical erythrol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical erythrol;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical erythrol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical erythrol;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of erythrol, or a composition comprising erythrol in said non-human organism or in the culture medium surrounding said non-human organism.

A further embodiment of the present invention relates to a process for the production of fructose, which comprises
(a1) increasing or generating the activity of a polypeptide comprising a polypeptide as depicted in the respective line in column 5 or 8 of Table II, application no. 17, or a homolog or fragment thereof, a consensus sequence or at least one polypeptide motif as depicted in column 8 of Table IV, application no. 17, whereby the respective line disclose in column 7 the fine chemical fructose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical fructose;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, which is joined to a transit peptide, whereby the respective line disclose in column 7 the fine chemical fructose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical fructose;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical fructose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical fructose;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of fructose, or a composition comprising fructose in said non-human organism or in the culture medium surrounding said non-human organism.

A further embodiment of the present invention relates to a process for the production of fucose, which comprises
(a1) increasing or generating the activity of a polypeptide comprising a polypeptide as depicted in the respective line in column 5 or 8 of Table II, application no. 17, or a homolog or fragment thereof, a consensus sequence or at least one polypeptide motif as depicted in column 8 of Table IV, application no. 17, whereby the respective line disclose in column 7 the fine chemical fucose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical fucose;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, which is joined to a transit peptide, whereby the respective line disclose in column 7 the fine chemical fucose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical fucose;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical fucose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical fucose;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of fucose, or a composition comprising fucose in said non-human organism or in the culture medium surrounding said non-human organism.

A further embodiment of the present invention relates to a process for the production of galactinol, which comprises
(a1) increasing or generating the activity of a polypeptide comprising a polypeptide as depicted in the respective line in column 5 or 8 of Table II, application no. 17, or a homolog or fragment thereof, a consensus sequence or at least one polypeptide motif as depicted in column 8 of Table IV, application no. 17, whereby the respective line disclose in column 7 the fine chemical galactinol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical galactinol;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, which is joined to a transit peptide, whereby the respective line disclose in column 7 the fine chemical galactinol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical galactinol;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical galactinol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical galactinol;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of galactinol, or a composition comprising galactinol in said non-human organism or in the culture medium surrounding said non-human organism.

A further embodiment of the present invention relates to a process for the production of galactitol, which comprises
(a1) increasing or generating the activity of a polypeptide comprising a polypeptide as depicted in the respective line in column 5 or 8 of Table II, application no. 17, or a homolog or fragment thereof, a consensus sequence or at least one polypeptide motif as depicted in column 8 of Table IV, application no. 17, whereby the respective line disclose in column 7 the fine chemical galactitol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical galactitol;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, which is joined to a transit peptide, whereby the respective line disclose in column 7 the fine chemical galactitol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical galactitol;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical galactitol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical galactitol;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of galactitol, or a composition comprising galactitol in said non-human organism or in the culture medium surrounding said non-human organism.

A further embodiment of the present invention relates to a process for the production of glucose, which comprises
(a1) increasing or generating the activity of a polypeptide comprising a polypeptide as depicted in the respective line in column 5 or 8 of Table II, application no. 17, or a homolog or fragment thereof, a consensus sequence or at least one polypeptide motif as depicted in column 8 of Table IV, application no. 17, whereby the respective line disclose in column 7 the fine chemical glucose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical glucose;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, which is joined to a transit peptide, whereby the respective line disclose in column 7 the fine chemical glucose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical glucose;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical glucose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical glucose;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of glucose, or a composition comprising glucose in said non-human organism or in the culture medium surrounding said non-human organism.

A further embodiment of the present invention relates to a process for the production of glucose-1-phosphate, which comprises
(a1) increasing or generating the activity of a polypeptide comprising a polypeptide as depicted in the respective line in column 5 or 8 of Table II, application no. 17, or a homolog or fragment thereof, a consensus sequence or at least one polypeptide motif as depicted in column 8 of Table IV, application no. 17, whereby the respective line disclose in column 7 the fine chemical glucose-1-phosphate; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical glucose-1-phosphate;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, which is joined to a transit peptide, whereby the respective line disclose in column 7 the fine chemical glucose-1-phosphate; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical glucose-1-phosphate;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical glucose-1-phosphate; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical glucose-1-phosphate;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of glucose-1-phosphate, or a composition comprising glucose-1-phosphate in said non-human organism or in the culture medium surrounding said non-human organism.

A further embodiment of the present invention relates to a process for the production of mannitol, which comprises
(a1) increasing or generating the activity of a polypeptide comprising a polypeptide as depicted in the respective line in column 5 or 8 of Table II, application no. 17, or a homolog or fragment thereof, a consensus sequence or at least one polypeptide motif as depicted in column 8 of Table IV, application no. 17, whereby the respective line disclose in column 7 the fine chemical mannitol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical mannitol;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, which is joined to a transit peptide, whereby the respective line disclose in column 7 the fine chemical mannitol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical mannitol;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical mannitol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical mannitol;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of mannitol, or a composition comprising mannitol in said non-human organism or in the culture medium surrounding said non-human organism.

A further embodiment of the present invention relates to a process for the production of melibiose, which comprises
(a1) increasing or generating the activity of a polypeptide comprising a polypeptide as depicted in the respective line in column 5 or 8 of Table II, application no. 17, or a homolog or fragment thereof, a consensus sequence or at least one polypeptide motif as depicted in column 8 of Table IV, application no. 17, whereby the respective line disclose in column 7 the fine chemical melibiose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical melibiose;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, which is joined to a transit peptide, whereby the respective line disclose in column 7 the fine chemical melibiose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical melibiose;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical melibiose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical melibiose;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of melibiose, or a composition comprising melibiose in said non-human organism or in the culture medium surrounding said non-human organism.

A further embodiment of the present invention relates to a process for the production of myo-inositol, which comprises
(a1) increasing or generating the activity of a polypeptide comprising a polypeptide as depicted in the respective line in column 5 or 8 of Table II, application no. 17, or a homolog or fragment thereof, a consensus sequence or at least one polypeptide motif as depicted in column 8 of Table IV, application no. 17, whereby the respective line disclose in column 7 the fine chemical myo-inositol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical myo-inositol;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, which is joined to a transit peptide, whereby the respective line disclose in column 7 the fine chemical myo-inositol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical myo-inositol;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical myo-inositol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical myo-inositol;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of myo-inositol, or a composition comprising myo-inositol in said non-human organism or in the culture medium surrounding said non-human organism.

A further embodiment of the present invention relates to a process for the production of raffinose, which comprises
(a1) increasing or generating the activity of a polypeptide comprising a polypeptide as depicted in the respective line in column 5 or 8 of Table II, application no. 17, or a homolog or fragment thereof, a consensus sequence or at least one polypeptide motif as depicted in column 8 of Table IV, application no. 17, whereby the respective line disclose in column 7 the fine chemical raffinose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical raffinose;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, which is joined to a transit peptide, whereby the respective line disclose in column 7 the fine chemical raffinose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical raffinose;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical raffinose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical raffinose;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of raffinose, or a composition comprising raffinose in said non-human organism or in the culture medium surrounding said non-human organism.

A further embodiment of the present invention relates to a process for the production of saccharic acid, which comprises
(a1) increasing or generating the activity of a polypeptide comprising a polypeptide as depicted in the respective line in column 5 or 8 of Table II, application no. 17, or a homolog or fragment thereof, a consensus sequence or at least one polypeptide motif as depicted in column 8 of Table IV, application no. 17, whereby the respective line disclose in column 7 the fine chemical saccharic acid; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical saccharic acid;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, which is joined to a transit peptide, whereby the respective line disclose in column 7 the fine chemical saccharic acid; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical saccharic acid;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical saccharic acid; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical saccharic acid;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of saccharic acid, or a composition comprising saccharic acid in said non-human organism or in the culture medium surrounding said non-human organism.

A further embodiment of the present invention relates to a process for the production of sedoheptulose-7-phosphate, which comprises
(a1) increasing or generating the activity of a polypeptide comprising a polypeptide as depicted in the respective line in column 5 or 8 of Table II, application no. 17, or a homolog or fragment thereof, a consensus sequence or at least one polypeptide motif as depicted in column 8 of Table IV, application no. 17, whereby the respective line disclose in column 7 the fine chemical sedoheptulose-7-phosphate; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical sedoheptulose-7-phosphate;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, which is joined to a transit peptide, whereby the respective line disclose in column 7 the fine chemical sedoheptulose-7-phosphate; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical sedoheptulose-7-phosphate;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical sedoheptulose-7-phosphate; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical sedoheptulose-7-phosphate;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of sedoheptulose-7-phosphate, or a composition comprising sedoheptulose-7-phosphate in said non-human organism or in the culture medium surrounding said non-human organism.

A further embodiment of the present invention relates to a process for the production of sorbitol, which comprises
(a1) increasing or generating the activity of a polypeptide comprising a polypeptide as depicted in the respective line in column 5 or 8 of Table II, application no. 17, or a homolog or fragment thereof, a consensus sequence or at least one polypeptide motif as depicted in column 8 of Table IV, application no. 17, whereby the respective line disclose in column 7 the fine chemical sorbitol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical sorbitol;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, which is joined to a transit peptide, whereby the respective line disclose in column 7 the fine chemical sorbitol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical sorbitol;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical sorbitol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical sorbitol;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of sorbitol, or a composition comprising sorbitol in said non-human organism or in the culture medium surrounding said non-human organism.

A further embodiment of the present invention relates to a process for the production of sucrose, which comprises
(a1) increasing or generating the activity of a polypeptide comprising a polypeptide as depicted in the respective line in column 5 or 8 of Table II, application no. 17, or a homolog or fragment thereof, a consensus sequence or at least one polypeptide motif as depicted in column 8 of Table IV, application no. 17, whereby the respective line disclose in column 7 the fine chemical sucrose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical sucrose;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, which is joined to a transit peptide, whereby the respective line disclose in column 7 the fine chemical sucrose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical sucrose;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical sucrose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical sucrose;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of sucrose, or a composition comprising sucrose in said non-human organism or in the culture medium surrounding said non-human organism.

A further embodiment of the present invention relates to a process for the production of trehalose, which comprises
(a1) increasing or generating the activity of a polypeptide comprising a polypeptide as depicted in the respective line in column 5 or 8 of Table II, application no. 17, or a homolog or fragment thereof, a consensus sequence or at least one polypeptide motif as depicted in column 8 of Table IV, application no. 17, whereby the respective line disclose in column 7 the fine chemical trehalose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical trehalose;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, which is joined to a transit peptide, whereby the respective line disclose in column 7 the fine chemical trehalose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical trehalose;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical trehalose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical trehalose;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of trehalose, or a composition comprising trehalose in said non-human organism or in the culture medium surrounding said non-human organism.

A further embodiment of the present invention relates to a process for the production of verbascose, which comprises
(a1) increasing or generating the activity of a polypeptide comprising a polypeptide as depicted in the respective line in column 5 or 8 of Table II, application no. 17, or a homolog or fragment thereof, a consensus sequence or at least one polypeptide motif as depicted in column 8 of Table IV, application no. 17, whereby the respective line disclose in column 7 the fine chemical verbascose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical verbascose;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, which is joined to a transit peptide, whereby the respective line disclose in column 7 the fine chemical verbascose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical verbascose;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical verbascose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical verbascose;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of verbascose, or a composition comprising verbascose in said non-human organism or in the culture medium surrounding said non-human organism.

Accordingly, the present invention relates to a process for the production of raffinose, which comprises
(a) increasing or generating the activity of a galactinol synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose-1-phosphate, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of glucose-1-phosphate, which comprises
(a) increasing or generating the activity of a At1g19350-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating the activity of a At1g19350-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of myo-inositol, which comprises
(a) increasing or generating the activity of a chlorophyllase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating the activity of a cullin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of fructose, which comprises
(a) increasing or generating the activity of a eukaryotic translation initiation factor 5 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a eukaryotic translation initiation factor 5 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of raffinose, which comprises
(a) increasing or generating the activity of a eukaryotic translation initiation factor 5 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sucrose, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of raffinose, which comprises
(a) increasing or generating the activity of a galactinol synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sucrose, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of saccharic acid, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

Accordingly, the present invention relates to a process for the production of fructose, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of saccharic acid, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

Accordingly, the present invention relates to a process for the production of fructose, which comprises
(a) increasing or generating the activity of a CBL-interacting protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a CBL-interacting protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of sedoheptulose-7-phosphate, which comprises
(a) increasing or generating the activity of a CBL-interacting protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of glucose-1-phosphate, which comprises
(a) increasing or generating the activity of a phenylalanine ammonia-lyase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating the activity of a MADS box protein transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a At2g45420-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of glucose-1-phosphate, which comprises
(a) increasing or generating the activity of a phosphatidylinositol 3- and 4-kinase family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of raffinose, which comprises
(a) increasing or generating the activity of a galactinol synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of raffinose, which comprises
(a) increasing or generating the activity of a galactinol synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of fructose, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of myo-inositol, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of fructose, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of myo-inositol, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating the activity of a thioredoxin family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a thioredoxin family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of myo-inositol, which comprises
(a) increasing or generating the activity of a thioredoxin family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of saccharic acid, which comprises
(a) increasing or generating the activity of a DNA binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

Accordingly, the present invention relates to a process for the production of glucose-1-phosphate, which comprises
(a) increasing or generating the activity of a CCAAT-binding transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of raffinose, which comprises
(a) increasing or generating the activity of a CCAAT-binding transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of fructose, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of sedoheptulose-7-phosphate, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of fructose, which comprises
(a) increasing or generating the activity of a eukaryotic translation initiation factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a eukaryotic translation initiation factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of raffinose, which comprises
(a) increasing or generating the activity of a eukaryotic translation initiation factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of myo-inositol, which comprises
(a) increasing or generating the activity of a mitogen-activated protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sedoheptulose-7-phosphate, which comprises
(a) increasing or generating the activity of a auxin response factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of fructose, which comprises
(a) increasing or generating the activity of a monthiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a monthiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a monthiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a monthiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sedoheptulose-7-phosphate, which comprises
(a) increasing or generating the activity of a monthiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of sucrose, which comprises
(a) increasing or generating the activity of a monthiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sedoheptulose-7-phosphate, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of sucrose, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose-1-phosphate, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of fructose, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sedoheptulose-7-phosphate, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of sucrose, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sedoheptulose-7-phosphate, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of sucrose, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sucrose, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of sedoheptulose-7-phosphate, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of sucrose, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of myo-inositol, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sedoheptulose-7-phosphate, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of sucrose, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose-1-phosphate, which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of raffinose, which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sucrose, which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose-1-phosphate, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of raffinose, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of myo-inositol, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sucrose, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of myo-inositol, which comprises
(a) increasing or generating the activity of a gibberellin 20-oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of myo-inositol, which comprises
(a) increasing or generating the activity of a inositol-3-phosphate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating the activity of a inositol-3-phosphate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of fructose, which comprises
(a) increasing or generating the activity of a At5g16650-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of fructose, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of myo-inositol, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sedoheptulose-7-phosphate, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of sucrose, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose-1-phosphate, which comprises
(a) increasing or generating the activity of a At5g43630-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose-1-phosphate, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating the activity of a RNA-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a RNA-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of fructose, which comprises
(a) increasing or generating the activity of a MADS-box transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of raffinose, which comprises
(a) increasing or generating the activity of a MADS-box transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of saccharic acid, which comprises
(a) increasing or generating the activity of a MADS-box transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

Accordingly, the present invention relates to a process for the production of glucose-1-phosphate, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of raffinose, which comprises
(a) increasing or generating the activity of a glycosyl transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sucrose, which comprises
(a) increasing or generating the activity of a sec-independent protein translocase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of 3,4,5-trihydroxypentanoic acid, which comprises
(a) increasing or generating the activity of a beta-hydroxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating the activity of a elongation factor Tu in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a elongation factor Tu in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of fructose, which comprises
(a) increasing or generating the activity of a ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of sucrose, which comprises
(a) increasing or generating the activity of a ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose-1-phosphate, which comprises
(a) increasing or generating the activity of a acyl-CoA synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose, which comprises
(a) increasing or generating the activity of a 2-oxoglutarate dehydrogenase E1 subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a 2-oxoglutarate dehydrogenase E1 subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a 2-oxoglutarate dehydrogenase E1 subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating the activity of a 2-oxoglutarate dehydrogenase E1 subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sedoheptulose-7-phosphate, which comprises
(a) increasing or generating the activity of a 2-oxoglutarate dehydrogenase E1 subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of raffinose, which comprises
(a) increasing or generating the activity of a GDP-mannose dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of fructose, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a ThiF family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating the activity of a CTP synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a CTP synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of sucrose, which comprises
(a) increasing or generating the activity of a CTP synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of raffinose, which comprises
(a) increasing or generating the activity of a protein-L-isoaspartate O-methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of myo-inositol, which comprises
(a) increasing or generating the activity of a recombinase A in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating the activity of a recombinase A in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of myo-inositol, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of raffinose, which comprises
(a) increasing or generating the activity of a enoyl-CoA hydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of fructose, which comprises
(a) increasing or generating the activity of a NADH-quinone oxidoreductase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose-1-phosphate, which comprises
(a) increasing or generating the activity of a NADH-quinone oxidoreductase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of sucrose, which comprises
(a) increasing or generating the activity of a NADH-quinone oxidoreductase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of 3,4,5-trihydroxypentanoic acid, which comprises
(a) increasing or generating the activity of a isopropylmalate isomerase large subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating the activity of a hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of sucrose, which comprises
(a) increasing or generating the activity of a hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating the activity of a glutamate-ammonia-ligase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a glutamate-ammonia-ligase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of sucrose, which comprises
(a) increasing or generating the activity of a glutamate-ammonia-ligase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating the activity of a fumarylacetoacetate hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of raffinose, which comprises
(a) increasing or generating the activity of a glycosyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of myo-inositol, which comprises
(a) increasing or generating the activity of a peptidyl-prolyl cis-trans isomerase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of 3,4,5-trihydroxypentanoic acid, which comprises
(a) increasing or generating the activity of a NAD(P)H dehydrogenase (quinone) in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating the activity of a electron transfer flavoprotein subunit beta in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a electron transfer flavoprotein subunit beta in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a purine nucleoside phosphorylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating the activity of a purine nucleoside phosphorylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of raffinose, which comprises
(a) increasing or generating the activity of a transcriptional regulator protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose-1-phosphate, which comprises
(a) increasing or generating the activity of a AX653549-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating the activity of a AY087308-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a AY087308-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of glucose-1-phosphate, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose, which comprises
(a) increasing or generating the activity of a threonine synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of sucrose, which comprises
(a) increasing or generating the activity of a threonine synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of 3,4,5-trihydroxypentanoic acid, which comprises
(a) increasing or generating the activity of a b0050-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of raffinose, which comprises
(a) increasing or generating the activity of a L-ribulose-5-phosphate 4-epimerase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sucrose, which comprises
(a) increasing or generating the activity of a glucose dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose-1-phosphate, which comprises
(a) increasing or generating the activity of a serine protease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a serine protease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sucrose, which comprises
(a) increasing or generating the activity of a acyl-CoA dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose-1-phosphate, which comprises
(a) increasing or generating the activity of a b0251-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of fructose, which comprises
(a) increasing or generating the activity of a betaine aldehyde dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of melibiose, which comprises
(a) increasing or generating the activity of a beta-galactosidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of melibiose or a composition comprising melibiose in said non-human organism or in the culture medium surrounding said non-human organism.

Accordingly, the present invention relates to a process for the production of glucose-1-phosphate, which comprises
(a) increasing or generating the activity of a b0362-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating the activity of a penicillin-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of fructose, which comprises
(a) increasing or generating the activity of a ATP-binding component of a transport system in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a ATP-binding component of a transport system in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose, which comprises
(a) increasing or generating the activity of a membrane transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of saccharic acid, which comprises
(a) increasing or generating the activity of a b0488-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

Accordingly, the present invention relates to a process for the production of galactinol, which comprises
(a) increasing or generating the activity of a glycerate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

Accordingly, the present invention relates to a process for the production of fructose, which comprises
(a) increasing or generating the activity of a isochorismate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a isochorismate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of raffinose, which comprises
(a) increasing or generating the activity of a isochorismate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sucrose, which comprises
(a) increasing or generating the activity of a isochorismate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of 3,4,5-trihydroxypentanoic acid, which comprises
(a) increasing or generating the activity of a B0644-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of saccharic acid, which comprises
(a) increasing or generating the activity of a pyrimidine-specific ribonucleoside hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

Accordingly, the present invention relates to a process for the production of erythrol, which comprises
(a) increasing or generating the activity of a asparagine synthetase B in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of erythrol or a composition comprising erythrol in said non-human organism or in the culture medium surrounding said non-human organism.

Accordingly, the present invention relates to a process for the production of myo-inositol, which comprises
(a) increasing or generating the activity of a sensor histidine kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of myo-inositol, which comprises
(a) increasing or generating the activity of a succinyl-CoA synthetase beta chain in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating the activity of a zinc transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a zinc transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a zinc transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of raffinose, which comprises
(a) increasing or generating the activity of a zinc transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose, which comprises
(a) increasing or generating the activity of a b0801-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of sedoheptulose-7-phosphate, which comprises
(a) increasing or generating the activity of a putrescine transport system permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of raffinose, which comprises
(a) increasing or generating the activity of a leucyl/phenylalanyl-tRNA-protein transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose-1-phosphate, which comprises
(a) increasing or generating the activity of a b0970-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a phosphoanhydride phosphorylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating the activity of a b1003-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a b1003-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a b1003-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of sedoheptulose-7-phosphate, which comprises
(a) increasing or generating the activity of a b1003-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of sucrose, which comprises
(a) increasing or generating the activity of a b1003-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of 3,4,5-trihydroxypentanoic acid, which comprises
(a) increasing or generating the activity of a b1046-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating the activity of a multidrug resistance protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of sucrose, which comprises
(a) increasing or generating the activity of a multidrug resistance protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating the activity of a outer membrane receptor protein precursor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose-1-phosphate, which comprises
(a) increasing or generating the activity of a adenylosuccinate lyase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose, which comprises
(a) increasing or generating the activity of a isocitrate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a b1137-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating the activity of a sodium/proton antiporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating the activity of a b1214-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating the activity of a b1214-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating the activity of a b1219-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a b1219-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of 3,4,5-trihydroxypentanoic acid, which comprises
(a) increasing or generating the activity of a b1219-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of myo-inositol, which comprises
(a) increasing or generating the activity of a b1234-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of galactinol, which comprises
(a) increasing or generating the activity of a cardiolipin synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

Accordingly, the present invention relates to a process for the production of galactitol, which comprises
(a) increasing or generating the activity of a cardiolipin synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactitol or a composition comprising galactitol in said non-human organism or in the culture medium surrounding said non-human organism.

Accordingly, the present invention relates to a process for the production of sorbitol, which comprises
(a) increasing or generating the activity of a cardiolipin synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sorbitol or a composition comprising sorbitol in said non-human organism or in the culture medium surrounding said non-human organism.

Accordingly, the present invention relates to a process for the production of 3,4,5-trihydroxypentanoic acid, which comprises
(a) increasing or generating the activity of a intracellular septation protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating the activity of a membrane protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a membrane protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of raffinose, which comprises
(a) increasing or generating the activity of a membrane protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sucrose, which comprises
(a) increasing or generating the activity of a membrane protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of myo-inositol, which comprises
(a) increasing or generating the activity of a tryptophan biosynthesis protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating the activity of a b1285-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose-1-phosphate, which comprises
(a) increasing or generating the activity of a phage-related repressor protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating the activity of a b1357-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose, which comprises
(a) increasing or generating the activity of a phenylacetic acid degradation protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of 3,4,5-trihydroxypentanoic acid, which comprises
(a) increasing or generating the activity of a phenylacetic acid degradation protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of sedoheptulose-7-phosphate, which comprises
(a) increasing or generating the activity of a b1410-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of myo-inositol, which comprises
(a) increasing or generating the activity of a b1425-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of glucose-1-phosphate, which comprises
(a) increasing or generating the activity of a lipoprotein precursor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of sedoheptulose-7-phosphate, which comprises
(a) increasing or generating the activity of a lipoprotein precursor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of 3,4,5-trihydroxypentanoic acid, which comprises
(a) increasing or generating the activity of a lipoprotein precursor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of 3,4,5-trihydroxypentanoic acid, which comprises
(a) increasing or generating the activity of a b1445-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating the activity of a b1470-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of fructose, which comprises
(a) increasing or generating the activity of a b1522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose-1-phosphate, which comprises
(a) increasing or generating the activity of a b1522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating the activity of a b1556-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of sedoheptulose-7-phosphate, which comprises
(a) increasing or generating the activity of a b1556-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of glucose-1-phosphate, which comprises
(a) increasing or generating the activity of a b1583-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating the activity of a Fe—S subunit of oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating the activity of a Fe—S subunit of oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating the activity of a acid shock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a acid shock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a acid shock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a acid shock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating the activity of a acid shock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of fructose, which comprises
(a) increasing or generating the activity of a phosphotransferase system component in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a phosphotransferase system component in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose, which comprises
(a) increasing or generating the activity of a electron transport complex protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a electron transport complex protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating the activity of a electron transport complex protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of myo-inositol, which comprises
(a) increasing or generating the activity of a methylglyoxalase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of myo-inositol, which comprises
(a) increasing or generating the activity of a b1670-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating the activity of a ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of myo-inositol, which comprises
(a) increasing or generating the activity of a transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating the activity of a b1796-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of myo-inositol, which comprises
(a) increasing or generating the activity of a para-aminobenzoate synthase component I in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of galactitol, which comprises
(a) increasing or generating the activity of a L-serine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactitol or a composition comprising galactitol in said non-human organism or in the culture medium surrounding said non-human organism.

Accordingly, the present invention relates to a process for the production of sorbitol, which comprises
(a) increasing or generating the activity of a L-serine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sorbitol or a composition comprising sorbitol in said non-human organism or in the culture medium surrounding said non-human organism.

Accordingly, the present invention relates to a process for the production of myo-inositol, which comprises
(a) increasing or generating the activity of a protease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of erythrol, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of erythrol or a composition comprising erythrol in said non-human organism or in the culture medium surrounding said non-human organism.

Accordingly, the present invention relates to a process for the production of myo-inositol, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sorbitol, which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sorbitol or a composition comprising sorbitol in said non-human organism or in the culture medium surrounding said non-human organism.

Accordingly, the present invention relates to a process for the production of fructose, which comprises
(a) increasing or generating the activity of a dATP pyrophosphohydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose-1-phosphate, which comprises
(a) increasing or generating the activity of a trehalose-6-phosphate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of trehalose, which comprises
(a) increasing or generating the activity of a trehalose-6-phosphate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of trehalose or a composition comprising trehalose in said non-human organism or in the culture medium surrounding said non-human organism.

Accordingly, the present invention relates to a process for the production of erythrol, which comprises
(a) increasing or generating the activity of a trehalose-phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of erythrol or a composition comprising erythrol in said non-human organism or in the culture medium surrounding said non-human organism.

Accordingly, the present invention relates to a process for the production of fructose, which comprises
(a) increasing or generating the activity of a trehalose-phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a trehalose-phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of sucrose, which comprises
(a) increasing or generating the activity of a trehalose-phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of raffinose, which comprises
(a) increasing or generating the activity of a b1898-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose-1-phosphate, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose, which comprises
(a) increasing or generating the activity of a flagellar M-ring protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of myo-inositol, which comprises
(a) increasing or generating the activity of a flagellar M-ring protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating the activity of a O-antigen chain length determinant in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a b2032-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of myo-inositol, which comprises
(a) increasing or generating the activity of a b2032-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of glucose-1-phosphate, which comprises
(a) increasing or generating the activity of a colanic acid biosynthesis protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of fucose, which comprises
(a) increasing or generating the activity of a GDP-mannose 4,6-dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fucose or a composition comprising fucose in said non-human organism or in the culture medium surrounding said non-human organism.

Accordingly, the present invention relates to a process for the production of fructose, which comprises
(a) increasing or generating the activity of a transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose-1-phosphate, which comprises
(a) increasing or generating the activity of a transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of raffinose, which comprises
(a) increasing or generating the activity of a transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sucrose, which comprises
(a) increasing or generating the activity of a transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of 3,4,5-trihydroxypentanoic acid, which comprises
(a) increasing or generating the activity of a transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating the activity of a b2107-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating the activity of a b2107-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating the activity of a b2139-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of erythrol, which comprises
(a) increasing or generating the activity of a sn-glycerol-3-phosphate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of erythrol or a composition comprising erythrol in said non-human organism or in the culture medium surrounding said non-human organism.

Accordingly, the present invention relates to a process for the production of fructose, which comprises
(a) increasing or generating the activity of a ATP synthase subunit E in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of galactinol, which comprises
(a) increasing or generating the activity of a aminotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

Accordingly, the present invention relates to a process for the production of myo-inositol, which comprises
(a) increasing or generating the activity of a b2345-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of 3,4,5-trihydroxypentanoic acid, which comprises
(a) increasing or generating the activity of a b2345-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of verbascose, which comprises
(a) increasing or generating the activity of a b2360-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of verbascose or a composition comprising verbascose in said non-human organism or in the culture medium surrounding said non-human organism.

Accordingly, the present invention relates to a process for the production of 3,4,5-trihydroxypentanoic acid, which comprises
(a) increasing or generating the activity of a b2360-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating the activity of a b2361-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating the activity of a b2361-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of sucrose, which comprises
(a) increasing or generating the activity of a ion-transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating the activity of a b2399-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a b2399-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a b2399-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a b2399-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating the activity of a b2399-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sedoheptulose-7-phosphate, which comprises
(a) increasing or generating the activity of a b2399-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of sucrose, which comprises
(a) increasing or generating the activity of a b2399-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of raffinose, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of myo-inositol, which comprises
(a) increasing or generating the activity of a cysteine synthase A in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of verbascose, which comprises
(a) increasing or generating the activity of a cysteine synthase A in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of verbascose or a composition comprising verbascose in said non-human organism or in the culture medium surrounding said non-human organism.

Accordingly, the present invention relates to a process for the production of glucose-1-phosphate, which comprises
(a) increasing or generating the activity of a b2466-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a b2466-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of 3,4,5-trihydroxypentanoic acid, which comprises
(a) increasing or generating the activity of a b2466-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating the activity of a b2474-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of raffinose, which comprises
(a) increasing or generating the activity of a 2,3-dihydroxy-2,3-dihydrophenylpropionatedehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sucrose, which comprises
(a) increasing or generating the activity of a putative transport system permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose-1-phosphate, which comprises
(a) increasing or generating the activity of a b2613-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a b2613-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of myo-inositol, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sucrose, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose-1-phosphate, which comprises
(a) increasing or generating the activity of a b2653-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of sedoheptulose-7-phosphate, which comprises
(a) increasing or generating the activity of a b2653-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of 3,4,5-trihydroxypentanoic acid, which comprises
(a) increasing or generating the activity of a b2653-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating the activity of a b2654-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating the activity of a b2654-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating the activity of a b2670-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating the activity of a b2673-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating the activity of a b2673-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of myo-inositol, which comprises
(a) increasing or generating the activity of a b2682-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of glucose-1-phosphate, which comprises
(a) increasing or generating the activity of a recombinase A in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating the activity of a murein transglycosylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating the activity of a b2739-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a b2739-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose, which comprises
(a) increasing or generating the activity of a 2-C-methyl-D-erythritol 4-phosphate cytidylyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of myo-inositol, which comprises
(a) increasing or generating the activity of a 2-C-methyl-D-erythritol 4-phosphate cytidylyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating the activity of a 2-C-methyl-D-erythritol 4-phosphate cytidylyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose-1-phosphate, which comprises
(a) increasing or generating the activity of a phosphoadenosine phosphosulfate reductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating the activity of a phosphoadenosine phosphosulfate reductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating the activity of a flavodoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a flavodoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a flavodoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a flavodoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of 3,4,5-trihydroxypentanoic acid, which comprises
(a) increasing or generating the activity of a flavodoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating the activity of a amino-acid acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose-1-phosphate, which comprises
(a) increasing or generating the activity of a amino-acid acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a amino-acid acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose, which comprises
(a) increasing or generating the activity of a arginine exporter protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of myo-inositol, which comprises
(a) increasing or generating the activity of a arginine exporter protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of myo-inositol, which comprises
(a) increasing or generating the activity of a b2932-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of erythrol, which comprises
(a) increasing or generating the activity of a L-serine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of erythrol or a composition comprising erythrol in said non-human organism or in the culture medium surrounding said non-human organism.

Accordingly, the present invention relates to a process for the production of myo-inositol, which comprises
(a) increasing or generating the activity of a b3151-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating the activity of a b3151-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sucrose, which comprises
(a) increasing or generating the activity of a b3151-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating the activity of a b3221-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a b3221-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of 3,4,5-trihydroxypentanoic acid, which comprises
(a) increasing or generating the activity of a b3221-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of myo-inositol, which comprises
(a) increasing or generating the activity of a b3246-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of myo-inositol, which comprises
(a) increasing or generating the activity of a acetyl CoA carboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating the activity of a methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of raffinose, which comprises
(a) increasing or generating the activity of a methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sucrose, which comprises
(a) increasing or generating the activity of a methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose, which comprises
(a) increasing or generating the activity of a ferredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of myo-inositol, which comprises
(a) increasing or generating the activity of a b3346-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating the activity of a b3346-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of 3,4,5-trihydroxypentanoic acid, which comprises
(a) increasing or generating the activity of a b3346-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating the activity of a phosphoenolpyruvate carboxykinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a phosphoenolpyruvate carboxykinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of 3,4,5-trihydroxypentanoic acid, which comprises
(a) increasing or generating the activity of a B3427-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating the activity of a glycogen synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a glycogen synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of myo-inositol, which comprises
(a) increasing or generating the activity of a b3442-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of 3,4,5-trihydroxypentanoic acid, which comprises
(a) increasing or generating the activity of a B3443-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of saccharic acid, which comprises
(a) increasing or generating the activity of a sn-glycerol-3-phosphate transport system permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

Accordingly, the present invention relates to a process for the production of glucose-1-phosphate, which comprises
(a) increasing or generating the activity of a high-affinity branched-chain amino acid transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a high-affinity branched-chain amino acid transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating the activity of a trehalase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of galactinol, which comprises
(a) increasing or generating the activity of a trehalase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

Accordingly, the present invention relates to a process for the production of fructose, which comprises
(a) increasing or generating the activity of a mannitol-1-phosphate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of mannitol, which comprises
(a) increasing or generating the activity of a mannitol-1-phosphate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of mannitol or a composition comprising mannitol in said non-human organism or in the culture medium surrounding said non-human organism.

Accordingly, the present invention relates to a process for the production of sorbitol, which comprises
(a) increasing or generating the activity of a mannitol-1-phosphate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sorbitol or a composition comprising sorbitol in said non-human organism or in the culture medium surrounding said non-human organism.

Accordingly, the present invention relates to a process for the production of galactinol, which comprises
(a) increasing or generating the activity of a ketol-acid reductoisomerase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

Accordingly, the present invention relates to a process for the production of myo-inositol, which comprises
(a) increasing or generating the activity of a b3777-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of myo-inositol, which comprises
(a) increasing or generating the activity of a b3817-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating the activity of a b3817-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of myo-inositol, which comprises
(a) increasing or generating the activity of a b3818-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of myo-inositol, which comprises
(a) increasing or generating the activity of a threonine efflux protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of myo-inositol, which comprises
(a) increasing or generating the activity of a glutamine synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of verbascose, which comprises
(a) increasing or generating the activity of a glutamine synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of verbascose or a composition comprising verbascose in said non-human organism or in the culture medium surrounding said non-human organism.

Accordingly, the present invention relates to a process for the production of glucose-1-phosphate, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of fructose, which comprises
(a) increasing or generating the activity of a b3989-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a b3989-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a b3989-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of raffinose, which comprises
(a) increasing or generating the activity of a b3989-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sucrose, which comprises
(a) increasing or generating the activity of a b3989-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of galactinol, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

Accordingly, the present invention relates to a process for the production of fructose, which comprises
(a) increasing or generating the activity of a b4029-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a b4029-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of sedoheptulose-7-phosphate, which comprises
(a) increasing or generating the activity of a b4029-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of glucose-1-phosphate, which comprises
(a) increasing or generating the activity of a chorismate lyase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating the activity of a chorismate lyase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating the activity of a b4050-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a b4050-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of myo-inositol, which comprises
(a) increasing or generating the activity of a b4050-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating the activity of a b4056-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of galactinol, which comprises
(a) increasing or generating the activity of a acetyl-coenzyme A synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

Accordingly, the present invention relates to a process for the production of glucose-1-phosphate, which comprises
(a) increasing or generating the activity of a lipoprotein precursor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating the activity of a lipoprotein precursor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of myo-inositol, which comprises
(a) increasing or generating the activity of a b4250-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating the activity of a b4250-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of fructose, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of fructose, which comprises
(a) increasing or generating the activity of a gluconate transport system permease 3 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of galactinol, which comprises
(a) increasing or generating the activity of a purine-nucleoside phosphorylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

Accordingly, the present invention relates to a process for the production of myo-inositol, which comprises
(a) increasing or generating the activity of a lysine decarboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sucrose, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose-1-phosphate, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose-1-phosphate, which comprises
(a) increasing or generating the activity of a GM02LC38418-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of raffinose, which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sucrose, which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of 3,4,5-trihydroxypentanoic acid, which comprises
(a) increasing or generating the activity of a GM02LC46-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of 3,4,5-trihydroxypentanoic acid, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of myo-inositol, which comprises
(a) increasing or generating the activity of a alkaline phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of verbascose, which comprises
(a) increasing or generating the activity of a flavodoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of verbascose or a composition comprising verbascose in said non-human organism or in the culture medium surrounding said non-human organism.

Accordingly, the present invention relates to a process for the production of sucrose, which comprises
(a) increasing or generating the activity of a sll0254-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of verbascose, which comprises
(a) increasing or generating the activity of a sll0281-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of verbascose or a composition comprising verbascose in said non-human organism or in the culture medium surrounding said non-human organism.

Accordingly, the present invention relates to a process for the production of myo-inositol, which comprises
(a) increasing or generating the activity of a sll0354-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of myo-inositol, which comprises
(a) increasing or generating the activity of a bifunctional protein (phosphoribosyltransferase and regulatory protein) in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of glucose-1-phosphate, which comprises
(a) increasing or generating the activity of a sll0418-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a sll0418-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of myo-inositol, which comprises
(a) increasing or generating the activity of a sll0418-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of saccharic acid, which comprises
(a) increasing or generating the activity of a sll0418-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

Accordingly, the present invention relates to a process for the production of 3,4,5-trihydroxypentanoic acid, which comprises
(a) increasing or generating the activity of a sll0418-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating the activity of a urease subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating the activity of a glucokinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of saccharic acid, which comprises
(a) increasing or generating the activity of a urease accessory protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of saccharic acid or a composition comprising saccharic acid in said non-human organism or in the culture medium surrounding said non-human organism.

Accordingly, the present invention relates to a process for the production of glucose-1-phosphate, which comprises
(a) increasing or generating the activity of a binding-protein-dependent transport systems inner membrane component in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating the activity of a binding-protein-dependent transport systems inner membrane component in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating the activity of a 3-deoxy-7-phosphoheptulonate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of fructose, which comprises
(a) increasing or generating the activity of a glycogen synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of sucrose, which comprises
(a) increasing or generating the activity of a glycogen synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose-1-phosphate, which comprises
(a) increasing or generating the activity of a coproporphyrinogen oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating the activity of a short chain dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of raffinose, which comprises
(a) increasing or generating the activity of a glycogen (starch) synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of myo-inositol, which comprises
(a) increasing or generating the activity of a carbamoyl-phosphate synthase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of 3,4,5-trihydroxypentanoic acid, which comprises
(a) increasing or generating the activity of a carbamoyl-phosphate synthase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of myo-inositol, which comprises
(a) increasing or generating the activity of a 4-alpha-glucanotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of 3,4,5-trihydroxypentanoic acid, which comprises
(a) increasing or generating the activity of a alanine dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose, which comprises
(a) increasing or generating the activity of a sll1761-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose, which comprises
(a) increasing or generating the activity of a acyl transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a cation-transporting ATPase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating the activity of a cation-transporting ATPase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a cation-transporting ATPase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating the activity of a cation-transporting ATPase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose-1-phosphate, which comprises
(a) increasing or generating the activity of a glycosidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of fructose, which comprises
(a) increasing or generating the activity of a precorrin methylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a precorrin methylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating the activity of a slr0600-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating the activity of a Glu/Leu/Phe/Val dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating the activity of a malate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of sucrose, which comprises
(a) increasing or generating the activity of a geranylgeranyl pyrophosphate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating the activity of a circadian clock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a circadian clock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a circadian clock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating the activity of a amine oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of fructose, which comprises
(a) increasing or generating the activity of a kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a Mg-protoporphyrin IX monomethyl ester oxidative cyclase 66 kD subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a permease protein of ABC transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of glucose-1-phosphate, which comprises
(a) increasing or generating the activity of a dihydrolipoamide dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of raffinose, which comprises
(a) increasing or generating the activity of a dihydrolipoamide dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of myo-inositol, which comprises
(a) increasing or generating the activity of a slr1107-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of glucose-1-phosphate, which comprises
(a) increasing or generating the activity of a fatty acid desaturase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating the activity of a CDP-diglyceride synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a CDP-diglyceride synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating the activity of a iron(III) dicitrate-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of sucrose, which comprises
(a) increasing or generating the activity of a photosystem I reaction center subunit XI in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating the activity of a photosystem II protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of fructose, which comprises
(a) increasing or generating the activity of a glutamine amidotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of myo-inositol, which comprises
(a) increasing or generating the activity of a glutamine amidotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of glucose-1-phosphate, which comprises
(a) increasing or generating the activity of a NADH dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of raffinose, which comprises
(a) increasing or generating the activity of a pyridoxal phosphate biosynthetic protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose-1-phosphate, which comprises
(a) increasing or generating the activity of a phosphoadenosine phosphosulfate reductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating the activity of a threonine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating the activity of a threonine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating the activity of a short-chain alcohol dehydrogenase family in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a short-chain alcohol dehydrogenase family in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a short-chain alcohol dehydrogenase family in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a short-chain alcohol dehydrogenase family in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sucrose, which comprises
(a) increasing or generating the activity of a short-chain alcohol dehydrogenase family in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of raffinose, which comprises
(a) increasing or generating the activity of a phosphate acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose-1-phosphate, which comprises
(a) increasing or generating the activity of a photosystem I reaction centre subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of raffinose, which comprises
(a) increasing or generating the activity of a photosystem I reaction centre subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of 3,4,5-trihydroxypentanoic acid, which comprises
(a) increasing or generating the activity of a photosystem I reaction centre subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating the activity of a Sec-independent protein translocase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a Sec-independent protein translocase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of sedoheptulose-7-phosphate, which comprises
(a) increasing or generating the activity of a Sec-independent protein translocase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of fructose, which comprises
(a) increasing or generating the activity of a cell division protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a cell division protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of sucrose, which comprises
(a) increasing or generating the activity of a cell division protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose-1-phosphate, which comprises
(a) increasing or generating the activity of a branched-chain amino acid ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating the activity of a branched-chain amino acid ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of myo-inositol, which comprises
(a) increasing or generating the activity of a 3-isopropylmalate dehydratase large subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating the activity of a multiple antibiotic resistance protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a multiple antibiotic resistance protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a multiple antibiotic resistance protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of fructose, which comprises
(a) increasing or generating the activity of a TTC1386-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a TTC1386-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of fructose, which comprises
(a) increasing or generating the activity of a homocitrate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose-1-phosphate, which comprises
(a) increasing or generating the activity of a homocitrate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of sucrose, which comprises
(a) increasing or generating the activity of a homocitrate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of raffinose, which comprises
(a) increasing or generating the activity of a permease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of 3,4,5-trihydroxypentanoic acid, which comprises
(a) increasing or generating the activity of a permease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating the activity of a oxidoreductase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating the activity of a XM_{—}473199-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sucrose, which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose-1-phosphate, which comprises
(a) increasing or generating the activity of a 3-deoxy-7-phosphoheptulonate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating the activity of a phosphate permease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating the activity of a phosphate permease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating the activity of a ycl027w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose-1-phosphate, which comprises
(a) increasing or generating the activity of a ycl027w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a serine/threonine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sucrose, which comprises
(a) increasing or generating the activity of a serine/threonine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of fructose, which comprises
(a) increasing or generating the activity of a phosphoglycerate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a phosphoglycerate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of raffinose, which comprises
(a) increasing or generating the activity of a YCR047W-A-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of 3,4,5-trihydroxypentanoic acid, which comprises
(a) increasing or generating the activity of a ycr102c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of galactinol, which comprises
(a) increasing or generating the activity of a isocitrate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactinol or a composition comprising galactinol in said non-human organism or in the culture medium surrounding said non-human organism.

Accordingly, the present invention relates to a process for the production of myo-inositol, which comprises
(a) increasing or generating the activity of a cell division control protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of myo-inositol, which comprises
(a) increasing or generating the activity of a cyclin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating the activity of a YDL159W-A-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose-1-phosphate, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating the activity of a serine/threonine-protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating the activity of a integral membrane protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating the activity of a ydl235c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a ydl235c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of 3,4,5-trihydroxypentanoic acid, which comprises
(a) increasing or generating the activity of a ydl235c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of sucrose, which comprises
(a) increasing or generating the activity of a coproporphyrinogen III oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose, which comprises
(a) increasing or generating the activity of a branched-chain amino acid permease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of fructose, which comprises
(a) increasing or generating the activity of a ydr183w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a ydr183w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a ydr507c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of fructose, which comprises
(a) increasing or generating the activity of a 1,4-alpha-glucan branching enzyme in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a 1,4-alpha-glucan branching enzyme in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of 3,4,5-trihydroxypentanoic acid, which comprises
(a) increasing or generating the activity of a YEL030C-A-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating the activity of a aldolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose, which comprises
(a) increasing or generating the activity of a O-acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of raffinose, which comprises
(a) increasing or generating the activity of a yer177w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of myo-inositol, which comprises
(a) increasing or generating the activity of a YFL019C-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sucrose, which comprises
(a) increasing or generating the activity of a YFL019C-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of sucrose, which comprises
(a) increasing or generating the activity of a hexokinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose-1-phosphate, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating the activity of a ygl096w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating the activity of a ygl096w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of raffinose, which comprises
(a) increasing or generating the activity of a ygl237c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of glucose-1-phosphate, which comprises
(a) increasing or generating the activity of a alcohol dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating the activity of a alcohol dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose, which comprises
(a) increasing or generating the activity of a yhl013c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a yhl013c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of glucose-1-phosphate, which comprises
(a) increasing or generating the activity of a 3-phosphoglycerate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of fructose, which comprises
(a) increasing or generating the activity of a YIL083C-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of myo-inositol, which comprises
(a) increasing or generating the activity of a DnaJ-like chaperone in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of 3,4,5-trihydroxypentanoic acid, which comprises
(a) increasing or generating the activity of a YJL127W-A-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating the activity of a inositol-3-phosphate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of galactitol, which comprises
(a) increasing or generating the activity of a inositol-3-phosphate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of galactitol or a composition comprising galactitol in said non-human organism or in the culture medium surrounding said non-human organism.

Accordingly, the present invention relates to a process for the production of myo-inositol, which comprises
(a) increasing or generating the activity of a inositol-3-phosphate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sorbitol, which comprises
(a) increasing or generating the activity of a hexose transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sorbitol or a composition comprising sorbitol in said non-human organism or in the culture medium surrounding said non-human organism.

Accordingly, the present invention relates to a process for the production of myo-inositol, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of glucose, which comprises
(a) increasing or generating the activity of a cystathionine gamma-synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of fructose, which comprises
(a) increasing or generating the activity of a fructose biphosphate aldolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of sedoheptulose-7-phosphate, which comprises
(a) increasing or generating the activity of a fructose biphosphate aldolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of glucose-1-phosphate, which comprises
(a) increasing or generating the activity of a acyl carrier protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of sucrose, which comprises
(a) increasing or generating the activity of a halotolerance protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose, which comprises
(a) increasing or generating the activity of a phosphoenolpyruvate carboxykinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose, which comprises
(a) increasing or generating the activity of a phosphoenolpyruvate carboxykinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of sucrose, which comprises
(a) increasing or generating the activity of a vacuolar sorting protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of glucose-1-phosphate, which comprises
(a) increasing or generating the activity of a thioredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of fructose, which comprises
(a) increasing or generating the activity of a hexose transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of verbascose, which comprises
(a) increasing or generating the activity of a purine biosynthesis protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of verbascose or a composition comprising verbascose in said non-human organism or in the culture medium surrounding said non-human organism.

Accordingly, the present invention relates to a process for the production of glucose-1-phosphate, which comprises
(a) increasing or generating the activity of a ymr324c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a molecular chaperone in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sucrose, which comprises
(a) increasing or generating the activity of a molecular chaperone in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of raffinose, which comprises
(a) increasing or generating the activity of a sterol O-acyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of myo-inositol, which comprises
(a) increasing or generating the activity of a yol160w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of glucose, which comprises
(a) increasing or generating the activity of a phosphohydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a histone acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating the activity of a subunit of COMPASS-protein complex in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating the activity of a ypl144w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating the activity of a ypl144w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating the activity of a ypr098c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of 3,4,5-trihydroxypentanoic acid, which comprises
(a) increasing or generating the activity of a ypr098c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of glucose-1-phosphate, which comprises
(a) increasing or generating the activity of a polyamine transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a polyamine transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating the activity of a Zm_{—}4842_BE510522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a Zm_{—}4842_BE510522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of myo-inositol, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of fructose, which comprises
(a) increasing or generating the activity of a 47266012-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a 47266012-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a 47266012-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a 47266012-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of sedoheptulose-7-phosphate, which comprises
(a) increasing or generating the activity of a 47266012-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of glucose, which comprises
(a) increasing or generating the activity of a 49747384_SOYBEAN-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a 49747384_SOYBEAN-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of myo-inositol, which comprises
(a) increasing or generating the activity of a 49747384_SOYBEAN-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of myo-inositol or a composition comprising myo-inositol in said non-human organism or in the culture medium surrounding said non-human organism.

Accordingly, the present invention relates to a process for the production of raffinose, which comprises
(a) increasing or generating the activity of a 49747384_SOYBEAN-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of raffinose or a composition comprising raffinose in said non-human organism or in the culture medium surrounding said non-human organism.

Accordingly, the present invention relates to a process for the production of sedoheptulose-7-phosphate, which comprises
(a) increasing or generating the activity of a 49747384_SOYBEAN-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of fructose, which comprises
(a) increasing or generating the activity of a glucose-6-phosphate 1-dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a glucose-6-phosphate 1-dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of glucose-1-phosphate, which comprises
(a) increasing or generating the activity of a glucose-6-phosphate 1-dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose-1-phosphate or a composition comprising glucose-1-phosphate in said non-human organism or in the culture medium surrounding said non-human organism.

Accordingly, the present invention relates to a process for the production of sedoheptulose-7-phosphate, which comprises
(a) increasing or generating the activity of a glucose-6-phosphate 1-dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sedoheptulose-7-phosphate or a composition comprising sedoheptulose-7-phosphate in said non-human organism or in the culture medium surrounding said non-human organism.

Accordingly, the present invention relates to a process for the production of sucrose, which comprises
(a) increasing or generating the activity of a glucose-6-phosphate 1-dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of sucrose or a composition comprising sucrose in said non-human organism or in the culture medium surrounding said non-human organism.

Accordingly, the present invention relates to a process for the production of 3,4,5-trihydroxypentanoic acid, which comprises
(a) increasing or generating the activity of a glucose-6-phosphate 1-dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid or a composition comprising 3,4,5-trihydroxypentanoic acid in said non-human organism or in the culture medium surrounding said non-human organism.

Accordingly, the present invention relates to a process for the production of fructose, which comprises
(a) increasing or generating the activity of a DNA replication complex GINS protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of fructose or a composition comprising fructose in said non-human organism or in the culture medium surrounding said non-human organism.

Accordingly, the present invention relates to a process for the production of glucose, which comprises
(a) increasing or generating the activity of a acetyl CoA synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glucose or a composition comprising glucose in said non-human organism or in the culture medium surrounding said non-human organism.

Accordingly, the present invention relates to a process for the production of 3,4,5-trihydroxypentanoic acid, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical 3,4,5-trihydroxypentanoic acid; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical 3,4,5-trihydroxypentanoic acid;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of 3,4,5-trihydroxypentanoic acid, or a composition comprising 3,4,5-trihydroxypentanoic acid in said non-human organism or in the culture medium surrounding said non-human organism.

Accordingly, the present invention relates to a process for the production of erythrol, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical erythrol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical erythrol;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of erythrol, or a composition comprising erythrol in said non-human organism or in the culture medium surrounding said non-human organism.

Accordingly, the present invention relates to a process for the production of fructose, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical fructose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical fructose;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of fructose, or a composition comprising fructose in said non-human organism or in the culture medium surrounding said non-human organism.

Accordingly, the present invention relates to a process for the production of fucose, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical fucose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical fucose;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of fucose, or a composition comprising fucose in said non-human organism or in the culture medium surrounding said non-human organism.

Accordingly, the present invention relates to a process for the production of galactinol, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical galactinol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical galactinol;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of galactinol, or a composition comprising galactinol in said non-human organism or in the culture medium surrounding said non-human organism.

Accordingly, the present invention relates to a process for the production of galactitol, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical galactitol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical galactitol;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of galactitol, or a composition comprising galactitol in said non-human organism or in the culture medium surrounding said non-human organism.

Accordingly, the present invention relates to a process for the production of glucose, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical glucose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical glucose;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of glucose, or a composition comprising glucose in said non-human organism or in the culture medium surrounding said non-human organism.

Accordingly, the present invention relates to a process for the production of glucose-1-phosphate, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical glucose-1-phosphate; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical glucose-1-phosphate;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of glucose-1-phosphate, or a composition comprising glucose-1-phosphate in said non-human organism or in the culture medium surrounding said non-human organism.

Accordingly, the present invention relates to a process for the production of mannitol, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical mannitol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical mannitol;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of mannitol, or a composition comprising mannitol in said non-human organism or in the culture medium surrounding said non-human organism.

Accordingly, the present invention relates to a process for the production of melibiose, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical melibiose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical melibiose;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of melibiose, or a composition comprising melibiose in said non-human organism or in the culture medium surrounding said non-human organism.

Accordingly, the present invention relates to a process for the production of myo-inositol, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical myo-inositol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical myo-inositol;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of myo-inositol, or a composition comprising myo-inositol in said non-human organism or in the culture medium surrounding said non-human organism.

Accordingly, the present invention relates to a process for the production of raffinose, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical raffinose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical raffinose;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of raffinose, or a composition comprising raffinose in said non-human organism or in the culture medium surrounding said non-human organism.

Accordingly, the present invention relates to a process for the production of saccharic acid, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical saccharic acid; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical saccharic acid;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of saccharic acid, or a composition comprising saccharic acid in said non-human organism or in the culture medium surrounding said non-human organism.

Accordingly, the present invention relates to a process for the production of sedoheptulose-7-phosphate, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical sedoheptulose-7-phosphate; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical sedoheptulose-7-phosphate;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of sedoheptulose-7-phosphate, or a composition comprising sedoheptulose-7-phosphate in said non-human organism or in the culture medium surrounding said non-human organism.

Accordingly, the present invention relates to a process for the production of sorbitol, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical sorbitol; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical sorbitol;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of sorbitol, or a composition comprising sorbitol in said non-human organism or in the culture medium surrounding said non-human organism.

Accordingly, the present invention relates to a process for the production of sucrose, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical sucrose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical sucrose;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of sucrose, or a composition comprising sucrose in said non-human organism or in the culture medium surrounding said non-human organism.

Accordingly, the present invention relates to a process for the production of trehalose, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical trehalose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical trehalose;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of trehalose, or a composition comprising trehalose in said non-human organism or in the culture medium surrounding said non-human organism.

Accordingly, the present invention relates to a process for the production of verbascose, which comprises
(a) increasing or generating the activity of a polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, whereby the respective line disclose in column 7 the fine chemical verbascose; or
increasing or generating the activity of an expression product of one or more nucleic acid molecule(s) comprising a polynucleotide as depicted in the respective line in column 5 or 8 of Table I, application no. 17, preferably the coding region thereof, or a homolog or a fragment thereof, whereby the respective line disclose in column 7 the fine chemical verbascose;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of verbascose, or a composition comprising verbascose in said non-human organism or in the culture medium surrounding said non-human organism.

In a further embodiment the activity of the polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, is increased or generated non-targeted in the above-mentioned process in a microorganism or plant or a part thereof.

In a further embodiment said polypeptide has the activity of the respective polypeptide represented by a protein comprising a polypeptide as depicted in the respective line in column 5 of Table II, application no. 17.

In a further embodiment the activity of the expression product of one or more nucleic acid molecule(s) comprising a polynucleotide as depicted in the respective line in column 5 or 8 of Table I, application no. 17, preferably the coding region thereof, or a homolog or a fragment thereof, is increased or generated non-targeted in the above-mentioned process in a microorganism or plant or a part thereof.

In a further embodiment the activity of the polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, is increased or generated in the above-mentioned process in an organelle preferably in plastids or mitochondria, especially in plastids, of a microorganism or plant.

In a further embodiment said polypeptide has the activity of the respective polypeptide represented by a protein comprising a polypeptide as depicted in the respective line in column 5 of Table II, application no. 17.

In a further embodiment the activity of the expression product of one or more nucleic acid molecule(s) comprising a polynucleotide as depicted in the respective line in column 5 or 8 of Table I, application no. 17, preferably the coding region thereof, or a homolog or a fragment thereof, is increased or generated in the above-mentioned process in an organelle, preferably in plasids or mitochondria, especially in plastids, of a microorganism or plant.

In a further embodiment the activity of the polypeptide comprising a polypeptide as depicted in the respective line in column 5 or 8 of Table II, application no. 17, or a homolog or a fragment thereof, a consensus sequence or at least one polypeptide motif as depicted in the respective line in column 8 of Table IV, application no. 17, is increased or generated in the above-mentioned process in the cyctosol of a cell, of a microorganism or plant.

In a further embodiment said polypeptide has the activity of the respective polypeptide represented by a protein comprising a polypeptide as depicted in the respective line in column 5 of Table II, application no. 17.

In a further embodiment the activity of the expression product of one or more nucleic acid molecule(s) comprising a polynucleotide as depicted in the respective line in column 5 or 8 of Table I, application no. 17, preferably the coding region thereof, or a homolog or a fragment thereof, is increased or generated in the above-mentioned process in the cytosol of a cell, of a microorganism or plant.

[0039.1.7.17] to [0066.1.7.17] for the disclosure of this paragraphs see [0039.1.7.7] to [0066.1.7.7] above

As mentioned above the nucleic acid sequences coding for the proteins as shown in Table II, application no. 17, column 5 or 8, or homologs or fragments thereof, are joined to a nucleic acid sequence encoding a plastidic transit peptide, e.g, if for the nucleic acid molecule in Table I, column 6 the term “plastidic” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide coding sequence is fused in frame to the nucleic acid sequence coding for proteins as shown in Table II, application no. 17, column 5 or 8, or homologs or fragments thereof, e.g., if for the nucleic acid molecule in Table I, column 6 the term “plastidic” is indicated.

[0068.1.7.17] to [0072.1.7.17] for the disclosure of this paragraph see [0068.1.7.7] to [0072.1.7.7] above

As mentioned above the nucleic acid sequences coding for the proteins as shown in the respective line in Table II, application no. 17, column 5 or 8, or homologs or fragments thereof, are joined to a nucleic acid sequence encoding a mitochondric transit peptide, e.g., if for the respective nucleic acid molecule in Table I, column 6 the term “mitochondric” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in the respective line in Table II, application no. 17, column 5 or 8, or homologs or fragments thereof, e.g., if for the nucleic acid molecule in the respective line in Table I, column 6 the term “mitochondric” is indicated.

for the disclosure of this paragraph see [0074.1.7.7] above

for the disclosure of this paragraph see [0075.1.7.7] above

Nucleic acid sequences coding for the transit peptides may be chemically synthesized either in part or wholly according to structure of transit peptide sequences disclosed in the prior art. Said natural or chemically synthesized sequences can be directly linked to the sequences encoding the mature protein or via a linker nucleic acid sequence, which may be typically less than 500 base pairs, preferably less than 450, 400, 350, 300, 250 or 200 base pairs, more preferably less than 150, 100, 90, 80, 70, 60, 50, 40 or 30 base pairs and most preferably less than 25, 20, 15, 12, 9, 6 or 3 base pairs in length and are in frame to the coding sequence. Furthermore favorable nucleic acid sequences encoding transit peptides may comprise sequences derived from more than one biological and/or chemical source and may include a nucleic acid sequence derived from the amino-terminal region of the mature protein, which in its native state is linked to the transit peptide. In a preferred embodiment of the invention said amino-terminal region of the mature protein is typically less than 150 amino acids, preferably less than 140, 130, 120, 110, 100 or 90 amino acids, more preferably less than 80, 70, 60, 50, 40, 35, 30, 25 or 20 amino acids and most preferably less than 19, 18, 17, 16, 15, 14, 13, 12, 11 or 10 amino acids in length. But even shorter or longer stretches are also possible. In addition target sequences, which facilitate the transport of proteins to other cell compartments such as the vacuole, endoplasmic reticulum, golgi complex, glyoxysomes, peroxisomes, mitochondria (in case of a plastidal transit peptide) or plastids, especially chloroplasts (in case of a mitochondric transit peptide) may be also part of the inventive nucleic acid sequence. The proteins translated from said inventive nucleic acid sequences are a kind of fusion proteins that means the nucleic acid sequences encoding the plastidal transit peptide for example the ones shown in Table a, preferably the last one of the Table a are joint to the respective nucleic acid sequences shown in Table I, application no. 17, columns 5 or 8, especially the coding region thereof, or homologs or fragments thereof, in case of nucleic acid sequences being expressed in plastids, and that means the nucleic acid sequences encoding the mitochondric transit peptide for example the ones shown in Table b, preferably the last one of the Table b are joint to the respective nucleic acid sequences shown in Table I, application no. 17, columns 5 or 8, especially the coding region thereof, or homologs or fragments thereof, in case of nucleic acid sequences being expressed in mitochondria. The person skilled in the art is able to join said sequences in a functional manner. Advantageously the transit peptide part is cleaved off from the protein part shown in Table II, application no. 17, columns 5 or 8, or a homolog or fragment thereof, during or after the transport into the organelles, preferably into the plastids or mitochondria, respectively.

All products of the cleavage of the preferred transit peptide shown in the last line of Table a have preferably the N-terminal amino acid sequences QIA CSS or QIA EFQLTT in front of the start methionine of the respective protein mentioned in Table II, application no. 17, column 5 or 8. or homologs or fragments thereof. Other short amino acid sequences of an range of 1 to 20 amino acids preferable 2 to 15 amino acids, more preferable 3 to 10 amino acids most preferably 4 to 8 amino acids are also possible in front of the start methionine of the protein mentioned in Table II, application no. 17, columns 5 or 8, or homologs or fragments thereof. In case of the amino acid sequence QIA CSS the three amino acids in front of the start methionine are stemming from the LIC (=ligatation independent cloning) cassette. Said short amino acid sequence is preferred in the case of the expression of E. coli genes. In case of the amino acid sequence QIA EFQLTT the six amino acids in front of the start methionine are stemming from the LIC cassette. Said short amino acid sequence is preferred in the case of the expression of S. cerevisiae genes. The skilled worker knowns that other short sequences are also useful in the expression of the respective nucleic acid molecule comprising a polynucleotide as depicted in Table I, application no. 17, column 5 or 8, preferably the coding region thereof, or a homolog or a fragment thereof. Furthermore the skilled worker is aware of the fact that there is not a need for such short sequences in the expression of the genes.

for the disclosure of this paragraph see [0077.1.7.7] above

for the disclosure of this paragraph see [0078.1.7.7] above

Alternatively to the targeting of the respective sequences shown in Table II, application no. 17, columns 5 or 8, or homologs or fragments thereof, preferably of sequences in general encoded in the nucleus with the aid of the targeting sequences mentioned for example in Table a alone, Table b alone, or in combination with other targeting sequences preferably into plastids or mitochondria, respectively, the nucleic acids of the invention can directly be introduced into the plastidal or mitochondrial genome. Therefore in a preferred embodiment the respective nucleic acid sequences shown in the respective line in Table I, application no. 17, columns 5 or 8, preferably the coding region thereof, or homologs or fragments thereof, are directly introduced and expressed in plastids e.g., if for the nucleic acid molecule in the respective line in Table I, column 6, the term “plastidic” is indicated, or are directly introduced and expressed in mitochondria e.g., if for the nucleic acid molecule in the respective line in Table I, column 6 the term “mitochondric” is indicated.

The term “introduced” in the context of this specification shall mean the insertion of a nucleic acid sequence into the organism by means of a “transfection”, “transduction” or preferably by “transformation”.

A plastid, such as a chloroplast, or a mitochondrion, has been “transformed” by an exogenous (preferably foreign) nucleic acid sequence if a nucleic acid sequence has been introduced into the plastid or mitochondrion that means that this sequence has crossed the membrane or the membranes of the plastid or of the mitochondrion. The exogenous, preferably foreign, DNA may be integrated (covalently linked) into plastid or mitochondrial DNA making up the genome of the plastid or mitochondrion, or it may remain unintegrated (e.g., by including a chloroplast origin of replication). “Stably” integrated DNA sequences are those, which are inherited through plastid or mitochondrion replication, thereby transferring new plastids or mitochondria, with the features of the integrated DNA sequence to the progeny.

[0080.1.7.17] to [0083.1.7.17] for the disclosure of these paragraphs see [0080.1.7.7] to [0083.1.7.7] above

For the process of the present invention it may be of great advantage that by transforming the plastids the intraspecies specific transgene flow is blocked, because a lot of species such as corn, cotton and rice have a strict maternal inheritance of plastids. By placing the respective genes specified in the respective line in Table I, application no. 17, columns 5 or 8, preferably the coding region, or homologs or fragments thereof, e.g. if for the nucleic acid molecule in the respective line in column 6 of Table I the term “plastidic” is indicated, in the plastids of plants, these genes will not be present in the pollen of said plants.

A further preferred embodiment of the invention relates to the use of so called “chloroplast localization sequences”, in which a first RNA sequence or molecule is capable of transporting or “chaperoning” a second RNA sequence, such as a RNA sequence transcribed from the sequences depicted in the respective line in Table I, application no. 17, columns 5 or 8, preferably the coding region thereof, or a homolog or a fragment thereof, or a sequence encoding a protein, as depicted in the respective line in Table II, application no. 17, columns 5 and 8, or a homolog or a fragment thereof, from an external environment inside a cell or outside a plastid into a chloroplast. In one embodiment the chloroplast localization signal is substantially similar or complementary to a complete or intact viroid sequence. The chloroplast localization signal may be encoded by a DNA sequence, which is transcribed into the chloroplast localization RNA.

The term “viroid” refers to a naturally occurring single stranded RNA molecule (Flores, C. R. Acad. Sci. III. 324 (10), 943 (2001)). Viroids usually contain about 200-500 nucleotides and generally exist as circular molecules. Examples of viroids that contain chloroplast localization signals include but are not limited to ASBVd, PLMVd, CChMVd and ELVd. The viroid sequence or a functional part of it can be fused to the sequences depicted in the respective line in Table I, application no. 17, columns 5 or 8, preferably the coding region thereof, or a homolog or a fragment thereof, or a sequence encoding a protein, as depicted in the respective line in Table II, application no. 17, columns 5 or 8, or a homolog or a fragment thereof, in such a manner that the viroid sequence transports a sequence transcribed from a sequence as depicted in the respective line in Table I, application no. 17 columns 5 or 8, preferably the coding region thereof, or a homolog or a fragment thereof, or a sequence encoding a protein as depicted in the respective line in Table II, application no. 17 columns 5 or 8, or a homolog or a fragment thereof, into the chloroplasts, e.g. if for the nucleic acid molecule in column 6 of Table I the term “plastidic” is indicated. A preferred embodiment uses a modified ASBVd (Navarro et al., Virology. 268 (1), 218 (2000)).

In a further specific embodiment the protein to be expressed in the plastids such as the proteins depicted in the respective line in Table II, application no. 17, columns 5 or 8, or a homolog or a fragment thereof, e.g. if for the nucleic acid molecule in the respective line in column 6 of Table I the term “plastidic” is indicated, are encoded by different nucleic acids. Such a method is disclosed in WO 2004/040973, which shall be incorporated by reference. WO 2004/040973 teaches a method, which relates to the translocation of an RNA corresponding to a gene or gene fragment into the chloroplast by means of a chloroplast localization sequence. The genes, which should be expressed in the plant or plant cells, are split into nucleic acid fragments, which are introduced into different compartments in the plant e.g. the nucleus, the plastids and/or mitochondria. Additionally plant cells are described in which the chloroplast contains a ribozyme fused at one end to an RNA encoding a fragment of a protein used in the inventive process such that the ribozyme can trans-splice the translocated fusion RNA to the RNA encoding the gene fragment to form and as the case may be reunite the nucleic acid fragments to an intact mRNA encoding a functional protein for example as disclosed in the respective line in Table II, application no. 17, columns 5 or 8, or a homolog or a fragment thereof.

In a preferred embodiment of the invention the nucleic acid sequences as shown in the respective line in Table I, application no. 17, columns 5 or 8, preferably the coding region thereof, or homologs or fragments thereof, e.g. if for the nucleic acid molecule in the respective line in column 6 of Table I the term “plastidic” is indicated, used in the inventive process are transformed into plastids, which are metabolically active. Those plastids should preferably maintain at a high copy number in the plant or plant tissue of interest, most preferably the chloroplasts found in green plant tissues, such as leaves or cotyledons or in seeds.

For a good expression in the plastids the nucleic acid sequences as shown in the respective line in Table I, application no. 17, columns 5 or 8, preferably the coding region thereof, or homologs or fragments thereof, e.g. if for the nucleic acid molecule in the respective line in column 6 of Table I the term “plastidic” is indicated, are introduced into an expression cassette using preferably a promoter and terminater, which are active in plastids preferably a chloroplast promoter. Examples of such promoters include the psbA promoter from the gene from spinach or pea, the rbcL promoter, and the atpB promoter from corn.

[0089.1.7.17] to [0092.1.7.17] for the disclosure of these paragraphs see [0089.1.7.7] to [0092.1.7.7] above

Advantageously the process for the production of the fine chemical 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, and/or verbascose leads to an enhanced production of the respective fine chemical. The terms “enhanced” or “increase” mean at least a 10%, 20%, 30%, 40% or 50%, preferably at least 60%, 70%, 80%, 90% or 100%, more preferably 150%, 200%, 300%, 400% or 500% higher production of the respective fine chemical 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, and/or verbascose in comparison to the wild-type as defined above, e.g. that means in comparison to a non-human organism without the aforementioned modification of the activity of a protein as shown in the respective line in Table II, application no. 17, column 5 or 8, or a fragment or a homolog thereof. The modification of the activity of a protein as shown in the respective line in Table II, application no. 17, column 5 or 8, or a homolog or a fragment thereof, or their combination can be achieved by joining the protein to a respective transit peptide, e.g. if for the respective encoding nucleic acid molecule in column 6 of Table I the term “plastidic” or “mitochondric” is indicated.

Surprisingly it was found, that the transgenic expression of a protein as shown in the respective line in Table II, application no. 17, column 5, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, conferred a production of or an increase in 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, and/or verbascose,respectively, to the transgenic non-human organism as compared to a corresponding non-transformed wild type.

In one embodiment the transgenic expression of a protein as shown in the respective line in Table II, application no. 17, column 5 or 8, or a homolog or a fragment thereof, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, confers a production of or an increase in 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose7-phosphate, sorbitol, sucrose, trehalose, and/or verbascose,respectively, to the transgenic non-human organism as compared to a corresponding non-transformed wild type.

Accordingly, in one embodiment, an increase of raffinose in a non-human organism, as compared to a corresponding non-transformed wild type non-human organism, is conferred in the process of the invention, if the activity of a polypeptide showing the activity of a galactinol synthase, or if the activity of the polypeptide Atgols2, preferably represented by SEQ ID NO. 142993, or a homolog or fragment thereof, or if the activity of a polypeptide encoded by a nucleic acid molecule comprising the nucleic acid SEQ ID NO. 142992, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 142992 or polypeptide SEQ ID NO. 142993, respectively, or a homolog or a fragment thereof, is increased or generated, or if the activity galactinol synthase is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 1186 to 8115-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a protein phosphatase, or if the activity of the polypeptide At1g07430, preferably represented by SEQ ID NO. 17452, or a homolog or fragment thereof, or if the activity of a polypeptide encoded by a nucleic acid molecule comprising the nucleic acid SEQ ID NO. 17451, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 17451 or polypeptide SEQ ID NO. 17452, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 27 to 90-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a protein phosphatase, or if the activity of the polypeptide At1g07430, preferably represented by SEQ ID NO. 17452, or a homolog or fragment thereof, or if the activity of a polypeptide encoded by a nucleic acid molecule comprising the nucleic acid SEQ ID NO. 17451, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 17451 or polypeptide SEQ ID NO. 17452, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 25 to 96-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a At1g19350-protein, or if the activity of the polypeptide At1g19350, preferably represented by SEQ ID NO. 140772, or a homolog or fragment thereof, or if the activity of a polypeptide encoded by a nucleic acid molecule comprising the nucleic acid SEQ ID NO. 140771, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 140771 or polypeptide SEQ ID NO. 140772, respectively, or a homolog or a fragment thereof, is increased or generated, or if the activity At1g19350-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 36 to 66-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a At1g19350-protein, or if the activity of the polypeptide At1g19350, preferably represented by SEQ ID NO. 140772, or a homolog or fragment thereof, or if the activity of a polypeptide encoded by a nucleic acid molecule comprising the nucleic acid SEQ ID NO. 140771, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 140771 or polypeptide SEQ ID NO. 140772, respectively, or a homolog or a fragment thereof, is increased or generated, or if the activity At1g19350-protein is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 42 to 73-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a chlorophyllase, or if the activity of the polypeptide At1g19670, preferably represented by SEQ ID NO. 113652, or a homolog or fragment thereof, or if the activity of a polypeptide encoded by a nucleic acid molecule comprising the nucleic acid SEQ ID NO. 113651, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 113651 or polypeptide SEQ ID NO. 113652, respectively, or a homolog or a fragment thereof, is increased or generated, or if the activity chlorophyllase is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 27 to 54-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a cullin, or if the activity of the polypeptide At1g26830, preferably represented by SEQ ID NO. 17702, or a homolog or fragment thereof, or if the activity of a polypeptide encoded by a nucleic acid molecule comprising the nucleic acid SEQ ID NO. 17701, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 17701 or polypeptide SEQ ID NO. 17702, respectively, or a homolog or a fragment thereof, is increased or generated, or if the activity cullin is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 107 to 121-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a eukaryotic translation initiation factor 5, or if the activity of the polypeptide At1g36730, preferably represented by SEQ ID NO. 17969, or a homolog or fragment thereof, or if the activity of a polypeptide encoded by a nucleic acid molecule comprising the nucleic acid SEQ ID NO. 17968, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 17968 or polypeptide SEQ ID NO. 17969, respectively, or a homolog or a fragment thereof, is increased or generated, or if the activity eukaryotic translation initiation factor 5 is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 119 to 725-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a eukaryotic translation initiation factor 5, or if the activity of the polypeptide At1g36730, preferably represented by SEQ ID NO. 17969, or a homolog or fragment thereof, or if the activity of a polypeptide encoded by a nucleic acid molecule comprising the nucleic acid SEQ ID NO. 17968, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 17968 or polypeptide SEQ ID NO. 17969, respectively, or a homolog or a fragment thereof, is increased or generated, or if the activity eukaryotic translation initiation factor 5 is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 95 to 312-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a eukaryotic translation initiation factor 5, or if the activity of the polypeptide At1g36730, preferably represented by SEQ ID NO. 17969, or a homolog or fragment thereof, or if the activity of a polypeptide encoded by a nucleic acid molecule comprising the nucleic acid SEQ ID NO. 17968, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 17968 or polypeptide SEQ ID NO. 17969, respectively, or a homolog or a fragment thereof, is increased or generated, or if the activity eukaryotic translation initiation factor 5 is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 57 to 150-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a transcriptional regulator, or if the activity of the polypeptide At1g43850, preferably represented by SEQ ID NO. 18071, or a homolog or fragment thereof, or if the activity of a polypeptide encoded by a nucleic acid molecule comprising the nucleic acid SEQ ID NO. 18070, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 18070 or polypeptide SEQ ID NO. 18071, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 40 to 87-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a galactinol synthase, or if the activity of the polypeptide At1g60470, preferably represented by SEQ ID NO. 140842, or a homolog or fragment thereof, or if the activity of a polypeptide encoded by a nucleic acid molecule comprising the nucleic acid SEQ ID NO. 140841, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 140841 or polypeptide SEQ ID NO. 140842, respectively, or a homolog or a fragment thereof, is increased or generated, or if the activity galactinol synthase is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 182 to 2025-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a protein phosphatase, or if the activity of the polypeptide At1g72770, preferably represented by SEQ ID NO. 19420, or a homolog or fragment thereof, or if the activity of a polypeptide encoded by a nucleic acid molecule comprising the nucleic acid SEQ ID NO. 19419, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 19419 or polypeptide SEQ ID NO. 19420, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 69 to 193-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a protein phosphatase, or if the activity of the polypeptide At1g72770, preferably represented by SEQ ID NO. 19420, or a homolog or fragment thereof, or if the activity of a polypeptide encoded by a nucleic acid molecule comprising the nucleic acid SEQ ID NO. 19419, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 19419 or polypeptide SEQ ID NO. 19420, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 19 to 102-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a protein phosphatase, or if the activity of the polypeptide At1g72770, preferably represented by SEQ ID NO. 19420, or a homolog or fragment thereof, or if the activity of a polypeptide encoded by a nucleic acid molecule comprising the nucleic acid SEQ ID NO. 19419, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 19419 or polypeptide SEQ ID NO. 19420, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 49 to 123-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a protein phosphatase, or if the activity of the polypeptide At1g72770, preferably represented by SEQ ID NO. 19420, or a homolog or fragment thereof, or if the activity of a polypeptide encoded by a nucleic acid molecule comprising the nucleic acid SEQ ID NO. 19419, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 19419 or polypeptide SEQ ID NO. 19420, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 55 to 339-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a protein phosphatase, or if the activity of the polypeptide At1g72770, preferably represented by SEQ ID NO. 19420, or a homolog or fragment thereof, or if the activity of a polypeptide encoded by a nucleic acid molecule comprising the nucleic acid SEQ ID NO. 19419, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 19419 or polypeptide SEQ ID NO. 19420, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 35 to 82-percent is conferred as compared to a corresponding non-transformed wild type non-human organism.

Accordingly, in one embodiment, an increase of saccharic acid in a non-human organism, as compared to a corresponding non-transformed wild type non-human organism, is conferred in the process of the invention, if the activity of a polypeptide showing the activity of a protein phosphatase, or if the activity of the polypeptide At1g78200, preferably represented by SEQ ID NO. 141014, or a homolog or fragment thereof, or if the activity of a polypeptide encoded by a nucleic acid molecule comprising the nucleic acid SEQ ID NO. 141013, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 141013 or polypeptide SEQ ID NO. 141014, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical saccharic acid. For example, an increase of the saccharic acid of at least 1 percent, particularly in a range of 49 to 143-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a transcription factor, or if the activity of the polypeptide At2g17560, preferably represented by SEQ ID NO. 19503, or a homolog or fragment thereof, or if the activity of a polypeptide encoded by a nucleic acid molecule comprising the nucleic acid SEQ ID NO. 19502, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 19502 or polypeptide SEQ ID NO. 19503, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 77 to 460-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a transcription factor, or if the activity of the polypeptide At2g17560, preferably represented by SEQ ID NO. 19503, or a homolog or fragment thereof, or if the activity of a polypeptide encoded by a nucleic acid molecule comprising the nucleic acid SEQ ID NO. 19502, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 19502 or polypeptide SEQ ID NO. 19503, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 67 to 304-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a transcription factor, or if the activity of the polypeptide At2g17560, preferably represented by SEQ ID NO. 19503, or a homolog or fragment thereof, or if the activity of a polypeptide encoded by a nucleic acid molecule comprising the nucleic acid SEQ ID NO. 19502, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 19502 or polypeptide SEQ ID NO. 19503, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 23 to 32-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a protein phosphatase, or if the activity of the polypeptide At2g25070, preferably represented by SEQ ID NO. 1299, or a homolog or fragment thereof, or if the activity of a polypeptide encoded by a nucleic acid molecule comprising the nucleic acid SEQ ID NO. 1298, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 1298 or polypeptide SEQ ID NO. 1299, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 19 to 37-percent is conferred as compared to a corresponding non-transformed wild type non-human organism.

Accordingly, in one embodiment, an increase of saccharic acid in a non-human organism, as compared to a corresponding non-transformed wild type non-human organism, is conferred in the process of the invention, if the activity of a polypeptide showing the activity of a glutaredoxin, or if the activity of the polypeptide At2g30540, preferably represented by SEQ ID NO. 20347, or a homolog or fragment thereof, or if the activity of a polypeptide encoded by a nucleic acid molecule comprising the nucleic acid SEQ ID NO. 20346, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 20346 or polypeptide SEQ ID NO. 20347, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical saccharic acid. For example, an increase of the saccharic acid of at least 1 percent, particularly in a range of 44 to 162-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a CBL-interacting protein kinase, or if the activity of the polypeptide At2g34180, preferably represented by SEQ ID NO. 20579, or a homolog or fragment thereof, or if the activity of a polypeptide encoded by a nucleic acid molecule comprising the nucleic acid SEQ ID NO. 20578, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 20578 or polypeptide SEQ ID NO. 20579, respectively, or a homolog or a fragment thereof, is increased or generated, or if the activity CBL-interacting protein kinase is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 820 to 7343-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a CBL-interacting protein kinase, or if the activity of the polypeptide At2g34180, preferably represented by SEQ ID NO. 20579, or a homolog or fragment thereof, or if the activity of a polypeptide encoded by a nucleic acid molecule comprising the nucleic acid SEQ ID NO. 20578, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 20578 or polypeptide SEQ ID NO. 20579, respectively, or a homolog or a fragment thereof, is increased or generated, or if the activity CBL-interacting protein kinase is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 339 to 2993-percent is conferred as compared to a corresponding non-transformed wild type non-human organism.

Accordingly, in one embodiment, an increase of sedoheptulose-7-phosphate in a non-human organism, as compared to a corresponding non-transformed wild type non-human organism, is conferred in the process of the invention, if the activity of a polypeptide showing the activity of a CBL-interacting protein kinase, or if the activity of the polypeptide At2g34180, preferably represented by SEQ ID NO. 20579, or a homolog or fragment thereof, or if the activity of a polypeptide encoded by a nucleic acid molecule comprising the nucleic acid SEQ ID NO. 20578, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 20578 or polypeptide SEQ ID NO. 20579, respectively, or a homolog or a fragment thereof, is increased or generated, or if the activity CBL-interacting protein kinase is increased or generated in a non-human organism, like a microorganism or a plant cell, plant or part thereof, especially with non-targeted localization, whereby the respective line disclose in column 7 the fine chemical sedoheptulose-7-phosphate. For example, an increase of the sedoheptulose-7-phosphate of at least 1 percent, particularly in a range of 190 to 580-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a phenylalanine ammonia-lyase, or if the activity of the polypeptide At2g37040, preferably represented by SEQ ID NO. 133043, or a homolog or fragment thereof, or if the activity of a polypeptide encoded by a nucleic acid molecule comprising the nucleic acid SEQ ID NO. 133042, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 133042 or polypeptide SEQ ID NO. 133043, respectively, or a homolog or a fragment thereof, is increased or generated, or if the activity phenylalanine ammonia-lyase is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 34 to 95-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a MADS box protein transcription factor, or if the activity of the polypeptide At2g42830, preferably represented by SEQ ID NO. 108728, or a homolog or fragment thereof, or if the activity of a polypeptide encoded by a nucleic acid molecule comprising the nucleic acid SEQ ID NO. 108727, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 108727 or polypeptide SEQ ID NO. 108728, respectively, or a homolog or a fragment thereof, is increased or generated, or if the activity MADS box protein transcription factor is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 33 to 85-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a At2g45420-protein, or if the activity of the polypeptide At2g45420, preferably represented by SEQ ID NO. 1624, or a homolog or fragment thereof, or if the activity of a polypeptide encoded by a nucleic acid molecule comprising the nucleic acid SEQ ID NO. 1623, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 1623 or polypeptide SEQ ID NO. 1624, respectively, or a homolog or a fragment thereof, is increased or generated, or if the activity At2g45420-protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 28 to 34-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a phosphatidylinositol 3- and 4-kinase family protein, or if the activity of the polypeptide At2g46500, preferably represented by SEQ ID NO. 21107, or a homolog or fragment thereof, or if the activity of a polypeptide encoded by a nucleic acid molecule comprising the nucleic acid SEQ ID NO. 21106, preferably the coding region thereof, or a homolog or fragment thereof, e.g.

derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 21106 or polypeptide SEQ ID NO. 21107, respectively, or a homolog or a fragment thereof, is increased or generated, or if the activity phosphatidylinositol 3- and 4-kinase family protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 19 to 26-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a galactinol synthase, or if the activity of the polypeptide At2g47180, preferably represented by SEQ ID NO. 141638, or a homolog or fragment thereof, or if the activity of a polypeptide encoded by a nucleic acid molecule comprising the nucleic acid SEQ ID NO. 141637, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 141637 or polypeptide SEQ ID NO. 141638, respectively, or a homolog or a fragment thereof, is increased or generated, or if the activity galactinol synthase is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 569 to 4907-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a galactinol synthase, or if the activity of the polypeptide At2g47180, preferably represented by SEQ ID NO. 141638, or a homolog or fragment thereof, or if the activity of a polypeptide encoded by a nucleic acid molecule comprising the nucleic acid SEQ ID NO. 141637, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 141637 or polypeptide SEQ ID NO. 141638, respectively, or a homolog or a fragment thereof, is increased or generated, or if the activity galactinol synthase is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 158 to 11191-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a heat shock transcription factor, or if the activity of the polypeptide At3g02990, preferably represented by SEQ ID NO. 68658, or a homolog or fragment thereof, or if the activity of a polypeptide encoded by a nucleic acid molecule comprising the nucleic acid SEQ ID NO. 68657, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 68657 or polypeptide SEQ ID NO. 68658, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 426 to 970-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a heat shock transcription factor, or if the activity of the polypeptide At3g02990, preferably represented by SEQ ID NO. 68658, or a homolog or fragment thereof, or if the activity of a polypeptide encoded by a nucleic acid molecule comprising the nucleic acid SEQ ID NO. 68657, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 68657 or polypeptide SEQ ID NO. 68658, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 58 to 465-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a heat shock transcription factor, or if the activity of the polypeptide At3g02990, preferably represented by SEQ ID NO. 68658, or a homolog or fragment thereof, or if the activity of a polypeptide encoded by a nucleic acid molecule comprising the nucleic acid SEQ ID NO. 68657, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 68657 or polypeptide SEQ ID NO. 68658, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 29 to 46-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a heat shock transcription factor, or if the activity of the polypeptide At3g02990, preferably represented by SEQ ID NO. 68658, or a homolog or fragment thereof, or if the activity of a polypeptide encoded by a nucleic acid molecule comprising the nucleic acid SEQ ID NO. 68657, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 68657 or polypeptide SEQ ID NO. 68658, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 117 to 1149-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a pyruvate kinase, or if the activity of the polypeptide At3g04050, preferably represented by SEQ ID NO. 21498, or a homolog or fragment thereof, or if the activity of a polypeptide encoded by a nucleic acid molecule comprising the nucleic acid SEQ ID NO. 21497, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 21497 or polypeptide SEQ ID NO. 21498, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line disclose in column 7 the fine chemical fructose. For example, an increase of the fructose of at least 1 percent, particularly in a range of 192 to 299-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a protein phosphatase, or if the activity of the polypeptide At3g06270, preferably represented by SEQ ID NO. 22016, or a homolog or fragment thereof, or if the activity of a polypeptide encoded by a nucleic acid molecule comprising the nucleic acid SEQ ID NO. 22015, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 22015 or polypeptide SEQ ID NO. 22016, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 85 to 185-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a protein phosphatase, or if the activity of the polypeptide At3g06270, preferably represented by SEQ ID NO. 22016, or a homolog or fragment thereof, or if the activity of a polypeptide encoded by a nucleic acid molecule comprising the nucleic acid SEQ ID NO. 22015, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 22015 or polypeptide SEQ ID NO. 22016, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 25 to 80-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a thioredoxin family protein, or if the activity of the polypeptide At3g08710, preferably represented by SEQ ID NO. 22250, or a homolog or fragment thereof, or if the activity of a polypeptide encoded by a nucleic acid molecule comprising the nucleic acid SEQ ID NO. 22249, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 22249 or polypeptide SEQ ID NO. 22250, respectively, or a homolog or a fragment thereof, is increased or generated, or if the activity thioredoxin family protein is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 93 to 182-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a thioredoxin family protein, or if the activity of the polypeptide At3g08710, preferably represented by SEQ ID NO. 22250, or a homolog or fragment thereof, or if the activity of a polypeptide encoded by a nucleic acid molecule comprising the nucleic acid SEQ ID NO. 22249, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 22249 or polypeptide SEQ ID NO. 22250, respectively, or a homolog or a fragment thereof, is increased or generated, or if the activity thioredoxin family protein is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 69 to 147-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a thioredoxin family protein, or if the activity of the polypeptide At3g08710, preferably represented by SEQ ID NO. 22250, or a homolog or fragment thereof, or if the activity of a polypeptide encoded by a nucleic acid molecule comprising the nucleic acid SEQ ID NO. 22249, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 22249 or polypeptide SEQ ID NO. 22250, respectively, or a homolog or a fragment thereof, is increased or generated, or if the activity thioredoxin family protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 32 to 123-percent is conferred as compared to a corresponding non-transformed wild type non-human organism.

Accordingly, in one embodiment, an increase of saccharic acid in a non-human organism, as compared to a corresponding non-transformed wild type non-human organism, is conferred in the process of the invention, if the activity of a polypeptide showing the activity of a DNA binding protein, or if the activity of the polypeptide At3g14230, preferably represented by SEQ ID NO. 22700, or a homolog or fragment thereof, or if the activity of a polypeptide encoded by a nucleic acid molecule comprising the nucleic acid SEQ ID NO. 22699, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 22699 or polypeptide SEQ ID NO. 22700, respectively, or a homolog or a fragment thereof, is increased or generated, or if the activity DNA binding protein is increased or generated in a non-human organism, like a microorganism or a plant cell, plant or part thereof, especially with plastidic localization, whereby the respective line disclose in column 7 the fine chemical saccharic acid. For example, an increase of the saccharic acid of at least 1 percent, particularly in a range of 57 to 182-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a CCAAT-binding transcription factor, or if the activity of the polypeptide At3g20910, preferably represented by SEQ ID NO. 22922, or a homolog or fragment thereof, or if the activity of a polypeptide encoded by a nucleic acid molecule comprising the nucleic acid SEQ ID NO. 22921, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 22921 or polypeptide SEQ ID NO. 22922, respectively, or a homolog or a fragment thereof, is increased or generated, or if the activity CCAAT-binding transcription factor is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 18 to 45-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a CCAAT-binding transcription factor, or if the activity of the polypeptide At3g20910, preferably represented by SEQ ID NO. 22922, or a homolog or fragment thereof, or if the activity of a polypeptide encoded by a nucleic acid molecule comprising the nucleic acid SEQ ID NO. 22921, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 22921 or polypeptide SEQ ID NO. 22922, respectively, or a homolog or a fragment thereof, is increased or generated, or if the activity CCAAT-binding transcription factor is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 55 to 114-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a protein kinase, or if the activity of the polypeptide At3g23000, preferably represented by SEQ ID NO. 1816, or a homolog or fragment thereof, or if the activity of a polypeptide encoded by a nucleic acid molecule comprising the nucleic acid SEQ ID NO. 1815, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 1815 or polypeptide SEQ ID NO. 1816, respectively, or a homolog or a fragment thereof, is increased or generated, or if the activity protein kinase is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 123 to 2049-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a protein kinase, or if the activity of the polypeptide At3g23000, preferably represented by SEQ ID NO. 1816, or a homolog or fragment thereof, or if the activity of a polypeptide encoded by a nucleic acid molecule comprising the nucleic acid SEQ ID NO. 1815, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 1815 or polypeptide SEQ ID NO. 1816, respectively, or a homolog or a fragment thereof, is increased or generated, or if the activity protein kinase is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 177 to 3162-percent is conferred as compared to a corresponding non-transformed wild type non-human organism.

Accordingly, in one embodiment, an increase of sedoheptulose-7-phosphate in a non-human organism, as compared to a corresponding non-transformed wild type non-human organism, is conferred in the process of the invention, if the activity of a polypeptide showing the activity of a protein kinase, or if the activity of the polypeptide At3g23000, preferably represented by SEQ ID NO. 1816, or a homolog or fragment thereof, or if the activity of a polypeptide encoded by a nucleic acid molecule comprising the nucleic acid SEQ ID NO. 1815, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 1815 or polypeptide SEQ ID NO. 1816, respectively, or a homolog or a fragment thereof, is increased or generated, or if the activity protein kinase is increased or generated in a non-human organism, like a microorganism or a plant cell, plant or part thereof, especially with non-targeted localization, whereby the respective line disclose in column 7 the fine chemical sedoheptulose-7-phosphate. For example, an increase of the sedoheptulose-7-phosphate of at least 1 percent, particularly in a range of 116 to 571-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a eukaryotic translation initiation factor, or if the activity of the polypeptide At3g26400, preferably represented by SEQ ID NO. 68728, or a homolog or fragment thereof, or if the activity of a polypeptide encoded by a nucleic acid molecule comprising the nucleic acid SEQ ID NO. 68727, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 68727 or polypeptide SEQ ID NO. 68728, respectively, or a homolog or a fragment thereof, is increased or generated, or if the activity eukaryotic translation initiation factor is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 79 to 583-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a eukaryotic translation initiation factor, or if the activity of the polypeptide At3g26400, preferably represented by SEQ ID NO. 68728, or a homolog or fragment thereof, or if the activity of a polypeptide encoded by a nucleic acid molecule comprising the nucleic acid SEQ ID NO. 68727, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 68727 or polypeptide SEQ ID NO. 68728, respectively, or a homolog or a fragment thereof, is increased or generated, or if the activity eukaryotic translation initiation factor is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 112 to 264-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a eukaryotic translation initiation factor, or if the activity of the polypeptide At3g26400, preferably represented by SEQ ID NO. 68728, or a homolog or fragment thereof, or if the activity of a polypeptide encoded by a nucleic acid molecule comprising the nucleic acid SEQ ID NO. 68727, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 68727 or polypeptide SEQ ID NO. 68728, respectively, or a homolog or a fragment thereof, is increased or generated, or if the activity eukaryotic translation initiation factor is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 89 to 129-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a mitogenactivated protein kinase, or if the activity of the polypeptide At3g59790, preferably represented by SEQ ID NO. 141759, or a homolog or fragment thereof, or if the activity of a polypeptide encoded by a nucleic acid molecule comprising the nucleic acid SEQ ID NO. 141758, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 141758 or polypeptide SEQ ID NO. 141759, respectively, or a homolog or a fragment thereof, is increased or generated, or if the activity mitogen-activated protein kinase is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 33 to 77-percent is conferred as compared to a corresponding non-transformed wild type non-human organism.

Accordingly, in one embodiment, an increase of sedoheptulose-7-phosphate in a non-human organism, as compared to a corresponding non-transformed wild type non-human organism, is conferred in the process of the invention, if the activity of a polypeptide showing the activity of a auxin response factor, or if the activity of the polypeptide At3g61830, preferably represented by SEQ ID NO. 2368, or a homolog or fragment thereof, or if the activity of a polypeptide encoded by a nucleic acid molecule comprising the nucleic acid SEQ ID NO. 2367, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 2367 or polypeptide SEQ ID NO. 2368, respectively, or a homolog or a fragment thereof, is increased or generated, or if the activity auxin response factor is increased or generated in a non-human organism, like a microorganism or a plant cell, plant or part thereof, especially with non-targeted localization, whereby the respective line disclose in column 7 the fine chemical sedoheptulose-7-phosphate. For example, an increase of the sedoheptulose-7-phosphate of at least 1 percent, particularly in a range of 54 to 87-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a monthiol glutaredoxin, or if the activity of the polypeptide At3g62930, preferably represented by SEQ ID NO. 2574, or a homolog or fragment thereof, or if the activity of a polypeptide encoded by a nucleic acid molecule comprising the nucleic acid SEQ ID NO. 2573, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 2573 or polypeptide SEQ ID NO. 2574, respectively, or a homolog or a fragment thereof, is increased or generated, or if the activity monthiol glutaredoxin is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 194 to 4745-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a monthiol glutaredoxin, or if the activity of the polypeptide At3g62930, preferably represented by SEQ ID NO. 2574, or a homolog or fragment thereof, or if the activity of a polypeptide encoded by a nucleic acid molecule comprising the nucleic acid SEQ ID NO. 2573, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 2573 or polypeptide SEQ ID NO. 2574, respectively, or a homolog or a fragment thereof, is increased or generated, or if the activity monthiol glutaredoxin is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 207 to 4142-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a monthiol glutaredoxin, or if the activity of the polypeptide At3g62930, preferably represented by SEQ ID NO. 2574, or a homolog or fragment thereof, or if the activity of a polypeptide encoded by a nucleic acid molecule comprising the nucleic acid SEQ ID NO. 2573, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 2573 or polypeptide SEQ ID NO. 2574, respectively, or a homolog or a fragment thereof, is increased or generated, or if the activity monthiol glutaredoxin is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 32 to 176-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a monthiol glutaredoxin, or if the activity of the polypeptide At3g62930, preferably represented by SEQ ID NO. 2574, or a homolog or fragment thereof, or if the activity of a polypeptide encoded by a nucleic acid molecule comprising the nucleic acid SEQ ID NO. 2573, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 2573 or polypeptide SEQ ID NO. 2574, respectively, or a homolog or a fragment thereof, is increased or generated, or if the activity monthiol glutaredoxin is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 36 to 118-percent is conferred as compared to a corresponding non-transformed wild type non-human organism.

Accordingly, in one embodiment, an increase of sedoheptulose-7-phosphate in a non-human organism, as compared to a corresponding non-transformed wild type non-human organism, is conferred in the process of the invention, if the activity of a polypeptide showing the activity of a monthiol glutaredoxin, or if the activity of the polypeptide At3g62930, preferably represented by SEQ ID NO. 2574, or a homolog or fragment thereof, or if the activity of a polypeptide encoded by a nucleic acid molecule comprising the nucleic acid SEQ ID NO. 2573, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 2573 or polypeptide SEQ ID NO. 2574, respectively, or a homolog or a fragment thereof, is increased or generated, or if the activity monthiol glutaredoxin is increased or generated in a non-human organism, like a microorganism or a plant cell, plant or part thereof, especially with non-targeted localization, whereby the respective line disclose in column 7 the fine chemical sedoheptulose-7-phosphate. For example, an increase of the sedoheptulose-7-phosphate of at least 1 percent, particularly in a range of 85 to 752-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a monthiol glutaredoxin, or if the activity of the polypeptide At3g62930, preferably represented by SEQ ID NO. 2574, or a homolog or fragment thereof, or if the activity of a polypeptide encoded by a nucleic acid molecule comprising the nucleic acid SEQ ID NO. 2573, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 2573 or polypeptide SEQ ID NO. 2574, respectively, or a homolog or a fragment thereof, is increased or generated, or if the activity monthiol glutaredoxin is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 58 to 129-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 623 to 3749-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 867 to 3374-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 31 to 141-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 46 to 95-percent is conferred as compared to a corresponding non-transformed wild type non-human organism.

Accordingly, in one embodiment, an increase of sedoheptulose-7-phosphate in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical sedoheptulose-7-phosphate. For example, an increase of the sedoheptulose-7-phosphate of at least 1 percent, particularly in a range of 167 to 848-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 49 to 114-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a transcription factor, or if the activity of the polypeptide At4g09960, preferably represented by SEQ ID NO. 109718, or a homolog or fragment thereof, or if the activity of a polypeptide encoded by a nucleic acid molecule comprising the nucleic acid SEQ ID NO. 109717, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 109717 or polypeptide SEQ ID NO. 109718, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 18 to 53-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 321 to 3175-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 231 to 2124-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 107 to 199-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 97 to 241-percent is conferred as compared to a corresponding non-transformed wild type non-human organism.

Accordingly, in one embodiment, an increase of sedoheptulose-7-phosphate in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical sedoheptulose7-phosphate. For example, an increase of the sedoheptulose-7-phosphate of at least 1 percent, particularly in a range of 183 to 692-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 41 to 106-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a monothiol glutaredoxin, or if the activity of the polypeptide At4g15670, preferably represented by SEQ ID NO. 3280, or a homolog or fragment thereof, or if the activity of a polypeptide encoded by a nucleic acid molecule comprising the nucleic acid SEQ ID NO. 3279, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 3279 or polypeptide SEQ ID NO. 3280, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 624 to 6054-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a monothiol glutaredoxin, or if the activity of the polypeptide At4g15670, preferably represented by SEQ ID NO. 3280, or a homolog or fragment thereof, or if the activity of a polypeptide encoded by a nucleic acid molecule comprising the nucleic acid SEQ ID NO. 3279, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 3279 or polypeptide SEQ ID NO. 3280, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 156 to 1091-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a monothiol glutaredoxin, or if the activity of the polypeptide At4g15670, preferably represented by SEQ ID NO. 3280, or a homolog or fragment thereof, or if the activity of a polypeptide encoded by a nucleic acid molecule comprising the nucleic acid SEQ ID NO. 3279, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 3279 or polypeptide SEQ ID NO. 3280, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 124 to 251-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a monothiol glutaredoxin, or if the activity of the polypeptide At4g15670, preferably represented by SEQ ID NO. 3280, or a homolog or fragment thereof, or if the activity of a polypeptide encoded by a nucleic acid molecule comprising the nucleic acid SEQ ID NO. 3279, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 3279 or polypeptide SEQ ID NO. 3280, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 123 to 337-percent is conferred as compared to a corresponding non-transformed wild type non-human organism.

Accordingly, in one embodiment, an increase of sedoheptulose-7-phosphate in a non-human organism, as compared to a corresponding non-transformed wild type non-human organism, is conferred in the process of the invention, if the activity of a polypeptide showing the activity of a monothiol glutaredoxin, or if the activity of the polypeptide At4g15670, preferably represented by SEQ ID NO. 3280, or a homolog or fragment thereof, or if the activity of a polypeptide encoded by a nucleic acid molecule comprising the nucleic acid SEQ ID NO. 3279, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 3279 or polypeptide SEQ ID NO. 3280, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical sedoheptulose7-phosphate. For example, an increase of the sedoheptulose-7-phosphate of at least 1 percent, particularly in a range of 82 to 1306-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a monothiol glutaredoxin, or if the activity of the polypeptide At4g15670, preferably represented by SEQ ID NO. 3280, or a homolog or fragment thereof, or if the activity of a polypeptide encoded by a nucleic acid molecule comprising the nucleic acid SEQ ID NO. 3279, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 3279 or polypeptide SEQ ID NO. 3280, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 38 to 91-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a monothiol glutaredoxin, or if the activity of the polypeptide At4g15690, preferably represented by SEQ ID NO. 23845, or a homolog or fragment thereof, or if the activity of a polypeptide encoded by a nucleic acid molecule comprising the nucleic acid SEQ ID NO. 23844, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 23844 or polypeptide SEQ ID NO. 23845, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 100 to 2161-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a monothiol glutaredoxin, or if the activity of the polypeptide At4g15690, preferably represented by SEQ ID NO. 23845, or a homolog or fragment thereof, or if the activity of a polypeptide encoded by a nucleic acid molecule comprising the nucleic acid SEQ ID NO. 23844, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 23844 or polypeptide SEQ ID NO. 23845, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 98 to 1660-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a monothiol glutaredoxin, or if the activity of the polypeptide At4g15690, preferably represented by SEQ ID NO. 23845, or a homolog or fragment thereof, or if the activity of a polypeptide encoded by a nucleic acid molecule comprising the nucleic acid SEQ ID NO. 23844, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 23844 or polypeptide SEQ ID NO. 23845, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 49 to 103-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a monothiol glutaredoxin, or if the activity of the polypeptide At4g15690, preferably represented by SEQ ID NO. 23845, or a homolog or fragment thereof, or if the activity of a polypeptide encoded by a nucleic acid molecule comprising the nucleic acid SEQ ID NO. 23844, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 23844 or polypeptide SEQ ID NO. 23845, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 50 to 145-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a monothiol glutaredoxin, or if the activity of the polypeptide At4g15690, preferably represented by SEQ ID NO. 23845, or a homolog or fragment thereof, or if the activity of a polypeptide encoded by a nucleic acid molecule comprising the nucleic acid SEQ ID NO. 23844, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 23844 or polypeptide SEQ ID NO. 23845, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 34 to 69-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a monothiol glutaredoxin, or if the activity of the polypeptide At4g15700, preferably represented by SEQ ID NO. 3655, or a homolog or fragment thereof, or if the activity of a polypeptide encoded by a nucleic acid molecule comprising the nucleic acid SEQ ID NO. 3654, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 3654 or polypeptide SEQ ID NO. 3655, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 722 to 1818-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a monothiol glutaredoxin, or if the activity of the polypeptide At4g15700, preferably represented by SEQ ID NO. 3655, or a homolog or fragment thereof, or if the activity of a polypeptide encoded by a nucleic acid molecule comprising the nucleic acid SEQ ID NO. 3654, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 3654 or polypeptide SEQ ID NO. 3655, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 469 to 1285-percent is conferred as compared to a corresponding non-transformed wild type non-human organism.

Accordingly, in one embodiment, an increase of sedoheptulose-7-phosphate in a non-human organism, as compared to a corresponding non-transformed wild type non-human organism, is conferred in the process of the invention, if the activity of a polypeptide showing the activity of a monothiol glutaredoxin, or if the activity of the polypeptide At4g15700, preferably represented by SEQ ID NO. 3655, or a homolog or fragment thereof, or if the activity of a polypeptide encoded by a nucleic acid molecule comprising the nucleic acid SEQ ID NO. 3654, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 3654 or polypeptide SEQ ID NO. 3655, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical sedoheptulose7-phosphate. For example, an increase of the sedoheptulose-7-phosphate of at least 1 percent, particularly in a range of 254 to 653-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a monothiol glutaredoxin, or if the activity of the polypeptide At4g15700, preferably represented by SEQ ID NO. 3655, or a homolog or fragment thereof, or if the activity of a polypeptide encoded by a nucleic acid molecule comprising the nucleic acid SEQ ID NO. 3654, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 3654 or polypeptide SEQ ID NO. 3655, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 83 to 229-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a protein phosphatase, or if the activity of the polypeptide At4g26080, preferably represented by SEQ ID NO. 68850, or a homolog or fragment thereof, or if the activity of a polypeptide encoded by a nucleic acid molecule comprising the nucleic acid SEQ ID NO. 68849, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 68849 or polypeptide SEQ ID NO. 68850, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 33 to 138-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a glutaredoxin, or if the activity of the polypeptide At4g33040, preferably represented by SEQ ID NO. 4103, or a homolog or fragment thereof, or if the activity of a polypeptide encoded by a nucleic acid molecule comprising the nucleic acid SEQ ID NO. 4102, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 4102 or polypeptide SEQ ID NO. 4103, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 79 to 387-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a glutaredoxin, or if the activity of the polypeptide At4g33040, preferably represented by SEQ ID NO. 4103, or a homolog or fragment thereof, or if the activity of a polypeptide encoded by a nucleic acid molecule comprising the nucleic acid SEQ ID NO. 4102, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 4102 or polypeptide SEQ ID NO. 4103, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 58 to 294-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a glutaredoxin, or if the activity of the polypeptide At4g33040, preferably represented by SEQ ID NO. 4103, or a homolog or fragment thereof, or if the activity of a polypeptide encoded by a nucleic acid molecule comprising the nucleic acid SEQ ID NO. 4102, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 4102 or polypeptide SEQ ID NO. 4103, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 19 to 65-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a glutaredoxin, or if the activity of the polypeptide At4g33040, preferably represented by SEQ ID NO. 4103, or a homolog or fragment thereof, or if the activity of a polypeptide encoded by a nucleic acid molecule comprising the nucleic acid SEQ ID NO. 4102, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 4102 or polypeptide SEQ ID NO. 4103, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 50 to 110-percent is conferred as compared to a corresponding non-transformed wild type non-human organism.

Accordingly, in one embodiment, an increase of sedoheptulose-7-phosphate in a non-human organism, as compared to a corresponding non-transformed wild type non-human organism, is conferred in the process of the invention, if the activity of a polypeptide showing the activity of a glutaredoxin, or if the activity of the polypeptide At4g33040, preferably represented by SEQ ID NO. 4103, or a homolog or fragment thereof, or if the activity of a polypeptide encoded by a nucleic acid molecule comprising the nucleic acid SEQ ID NO. 4102, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 4102 or polypeptide SEQ ID NO. 4103, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical sedoheptulose-7-phosphate. For example, an increase of the sedoheptulose-7-phosphate of at least 1 percent, particularly in a range of 47 to 108-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a glutaredoxin, or if the activity of the polypeptide At4g33040, preferably represented by SEQ ID NO. 4103, or a homolog or fragment thereof, or if the activity of a polypeptide encoded by a nucleic acid molecule comprising the nucleic acid SEQ ID NO. 4102, preferably the coding region thereof, or a homolog or fragment thereof, e.g. derived from Arabidopsis thaliana, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 4102 or polypeptide SEQ ID NO. 4103, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 28 to 74-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a cyclin D , or if the activity of the polypeptide At4g34160, preferably represented by SEQ ID NO. 24312, or a homolog or fragment thereof, or if the activity of a polypeptide encoded by a nucleic acid molecule comprising the nucleic acid SEQ ID NO. 24311, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 24311 or polypeptide SEQ ID NO. 24312, respectively, or a homolog or a fragment thereof, is increased or generated, or if the activity cyclin D is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 20 to 47-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a cyclin D , or if the activity of the polypeptide At4g34160, preferably represented by SEQ ID NO. 24312, or a homolog or fragment thereof, or if the activity of a polypeptide encoded by a nucleic acid molecule comprising the nucleic acid SEQ ID NO. 24311, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 24311 or polypeptide SEQ ID NO. 24312, respectively, or a homolog or a fragment thereof, is increased or generated, or if the activity cyclin D is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 131 to 327-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a cyclin D , or if the activity of the polypeptide At4g34160, preferably represented by SEQ ID NO. 24312, or a homolog or fragment thereof, or if the activity of a polypeptide encoded by a nucleic acid molecule comprising the nucleic acid SEQ ID NO. 24311, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 24311 or polypeptide SEQ ID NO. 24312, respectively, or a homolog or a fragment thereof, is increased or generated, or if the activity cyclin D is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 31 to 97-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 112 to 350-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 32 to 55-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 66 to 186-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a protein phosphatase, or if the activity of the polypeptide At5g02760, preferably represented by SEQ ID NO. 68988, or a homolog or fragment thereof, or if the activity of a polypeptide encoded by a nucleic acid molecule comprising the nucleic acid SEQ ID NO. 68987, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 68987 or polypeptide SEQ ID NO. 68988, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 25 to 104-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a heat shock transcription factor, or if the activity of the polypeptide At5g03720, preferably represented by SEQ ID NO. 24439, or a homolog or fragment thereof, or if the activity of a polypeptide encoded by a nucleic acid molecule comprising the nucleic acid SEQ ID NO. 24438, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 24438 or polypeptide SEQ ID NO. 24439, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 134 to 1766-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a heat shock transcription factor, or if the activity of the polypeptide At5g03720, preferably represented by SEQ ID NO. 24439, or a homolog or fragment thereof, or if the activity of a polypeptide encoded by a nucleic acid molecule comprising the nucleic acid SEQ ID NO. 24438, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 24438 or polypeptide SEQ ID NO. 24439, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 106 to 2148-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a heat shock transcription factor, or if the activity of the polypeptide At5g03720, preferably represented by SEQ ID NO. 24439, or a homolog or fragment thereof, or if the activity of a polypeptide encoded by a nucleic acid molecule comprising the nucleic acid SEQ ID NO. 24438, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 24438 or polypeptide SEQ ID NO. 24439, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 28 to 106-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a heat shock transcription factor, or if the activity of the polypeptide At5g03720, preferably represented by SEQ ID NO. 24439, or a homolog or fragment thereof, or if the activity of a polypeptide encoded by a nucleic acid molecule comprising the nucleic acid SEQ ID NO. 24438, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 24438 or polypeptide SEQ ID NO. 24439, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 92 to 427-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a heat shock transcription factor, or if the activity of the polypeptide At5g03720, preferably represented by SEQ ID NO. 24439, or a homolog or fragment thereof, or if the activity of a polypeptide encoded by a nucleic acid molecule comprising the nucleic acid SEQ ID NO. 24438, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 24438 or polypeptide SEQ ID NO. 24439, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 1744 to 12039-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a heat shock transcription factor, or if the activity of the polypeptide At5g03720, preferably represented by SEQ ID NO. 24439, or a homolog or fragment thereof, or if the activity of a polypeptide encoded by a nucleic acid molecule comprising the nucleic acid SEQ ID NO. 24438, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 24438 or polypeptide SEQ ID NO. 24439, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 35 to 100-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a gibberellin 20-oxidase, or if the activity of the polypeptide At5g07200, preferably represented by SEQ ID NO. 24493, or a homolog or fragment thereof, or if the activity of a polypeptide encoded by a nucleic acid molecule comprising the nucleic acid SEQ ID NO. 24492, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 24492 or polypeptide SEQ ID NO. 24493, respectively, or a homolog or a fragment thereof, is increased or generated, or if the activity gibberellin 20-oxidase is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 24 to 47-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a inositol-3-phosphate synthase, or if the activity of the polypeptide At5g10170, preferably represented by SEQ ID NO. 142754, or a homolog or fragment thereof, or if the activity of a polypeptide encoded by a nucleic acid molecule comprising the nucleic acid SEQ ID NO. 142753, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 142753 or polypeptide SEQ ID NO. 142754, respectively, or a homolog or a fragment thereof, is increased or generated, or if the activity inositol-3-phosphate synthase is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 83 to 386-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a inositol-3-phosphate synthase, or if the activity of the polypeptide At5g10170, preferably represented by SEQ ID NO. 142754, or a homolog or fragment thereof, or if the activity of a polypeptide encoded by a nucleic acid molecule comprising the nucleic acid SEQ ID NO. 142753, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 142753 or polypeptide SEQ ID NO. 142754, respectively, or a homolog or a fragment thereof, is increased or generated, or if the activity inositol-3-phosphate synthase is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 72 to 155-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a glutaredoxin, or if the activity of the polypeptide At5g14070, preferably represented by SEQ ID NO. 69276, or a homolog or fragment thereof, or if the activity of a polypeptide encoded by a nucleic acid molecule comprising the nucleic acid SEQ ID NO. 69275, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 69275 or polypeptide SEQ ID NO. 69276, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 70 to 248-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a At5g16650-protein, or if the activity of the polypeptide At5g16650, preferably represented by SEQ ID NO. 25284, or a homolog or fragment thereof, or if the activity of a polypeptide encoded by a nucleic acid molecule comprising the nucleic acid SEQ ID NO. 25283, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 25283 or polypeptide SEQ ID NO. 25284, respectively, or a homolog or a fragment thereof, is increased or generated, or if the activity At5g16650-protein is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 80 to 153-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a monothiol glutaredoxin, or if the activity of the polypeptide At5g18600, preferably represented by SEQ ID NO. 4905, or a homolog or fragment thereof, or if the activity of a polypeptide encoded by a nucleic acid molecule comprising the nucleic acid SEQ ID NO. 4904, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 4904 or polypeptide SEQ ID NO. 4905, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 228 to 2679-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a monothiol glutaredoxin, or if the activity of the polypeptide At5g18600, preferably represented by SEQ ID NO. 4905, or a homolog or fragment thereof, or if the activity of a polypeptide encoded by a nucleic acid molecule comprising the nucleic acid SEQ ID NO. 4904, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 4904 or polypeptide SEQ ID NO. 4905, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 187 to 2197-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a monothiol glutaredoxin, or if the activity of the polypeptide At5g18600, preferably represented by SEQ ID NO. 4905, or a homolog or fragment thereof, or if the activity of a polypeptide encoded by a nucleic acid molecule comprising the nucleic acid SEQ ID NO. 4904, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 4904 or polypeptide SEQ ID NO. 4905, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 46 to 76-percent is conferred as compared to a corresponding non-transformed wild type non-human organism.

Accordingly, in one embodiment, an increase of sedoheptulose-7-phosphate in a non-human organism, as compared to a corresponding non-transformed wild type non-human organism, is conferred in the process of the invention, if the activity of a polypeptide showing the activity of a monothiol glutaredoxin, or if the activity of the polypeptide At5g18600, preferably represented by SEQ ID NO. 4905, or a homolog or fragment thereof, or if the activity of a polypeptide encoded by a nucleic acid molecule comprising the nucleic acid SEQ ID NO. 4904, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 4904 or polypeptide SEQ ID NO. 4905, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical sedoheptulose7-phosphate. For example, an increase of the sedoheptulose-7-phosphate of at least 1 percent, particularly in a range of 149 to 336-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a monothiol glutaredoxin, or if the activity of the polypeptide At5g18600, preferably represented by SEQ ID NO. 4905, or a homolog or fragment thereof, or if the activity of a polypeptide encoded by a nucleic acid molecule comprising the nucleic acid SEQ ID NO. 4904, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 4904 or polypeptide SEQ ID NO. 4905, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 40 to 141-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a transcription factor, or if the activity of the polypeptide At5g39760, preferably represented by SEQ ID NO. 25429, or a homolog or fragment thereof, or if the activity of a polypeptide encoded by a nucleic acid molecule comprising the nucleic acid SEQ ID NO. 25428, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 25428 or polypeptide SEQ ID NO. 25429, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 114 to 219-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a At5g43630-protein, or if the activity of the polypeptide At5g43630, preferably represented by SEQ ID NO. 142930, or a homolog or fragment thereof, or if the activity of a polypeptide encoded by a nucleic acid molecule comprising the nucleic acid SEQ ID NO. 142929, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 142929 or polypeptide SEQ ID NO. 142930, respectively, or a homolog or a fragment thereof, is increased or generated, or if the activity At5g43630-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 65 to 74-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a heat shock transcription factor, or if the activity of the polypeptide At5g54070, preferably represented by SEQ ID NO. 142934, or a homolog or fragment thereof, or if the activity of a polypeptide encoded by a nucleic acid molecule comprising the nucleic acid SEQ ID NO. 142933, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 142933 or polypeptide SEQ ID NO. 142934, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 30 to 65-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a heat shock transcription factor, or if the activity of the polypeptide At5g54070, preferably represented by SEQ ID NO. 142934, or a homolog or fragment thereof, or if the activity of a polypeptide encoded by a nucleic acid molecule comprising the nucleic acid SEQ ID NO. 142933, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 142933 or polypeptide SEQ ID NO. 142934, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 68 to 383-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a protein phosphatase, or if the activity of the polypeptide At5g57050, preferably represented by SEQ ID NO. 5319, or a homolog or fragment thereof, or if the activity of a polypeptide encoded by a nucleic acid molecule comprising the nucleic acid SEQ ID NO. 5318, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 5318 or polypeptide SEQ ID NO. 5319, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 37 to 73-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a protein phosphatase, or if the activity of the polypeptide At5g57050, preferably represented by SEQ ID NO. 5319, or a homolog or fragment thereof, or if the activity of a polypeptide encoded by a nucleic acid molecule comprising the nucleic acid SEQ ID NO. 5318, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 5318 or polypeptide SEQ ID NO. 5319, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 27 to 112-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a RNA-binding protein, or if the activity of the polypeptide At5g60110, preferably represented by SEQ ID NO. 70007, or a homolog or fragment thereof, or if the activity of a polypeptide encoded by a nucleic acid molecule comprising the nucleic acid SEQ ID NO. 70006, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 70006 or polypeptide SEQ ID NO. 70007, respectively, or a homolog or a fragment thereof, is increased or generated, or if the activity RNA-binding protein is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 90 to 185-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a RNA-binding protein, or if the activity of the polypeptide At5g60110, preferably represented by SEQ ID NO. 70007, or a homolog or fragment thereof, or if the activity of a polypeptide encoded by a nucleic acid molecule comprising the nucleic acid SEQ ID NO. 70006, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 70006 or polypeptide SEQ ID NO. 70007, respectively, or a homolog or a fragment thereof, is increased or generated, or if the activity RNA-binding protein is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 68 to 191-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a MADS-box transcription factor, or if the activity of the polypeptide At5g60440, preferably represented by SEQ ID NO. 102900, or a homolog or fragment thereof, or if the activity of a polypeptide encoded by a nucleic acid molecule comprising the nucleic acid SEQ ID NO. 102899, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 102899 or polypeptide SEQ ID NO. 102900, respectively, or a homolog or a fragment thereof, is increased or generated, or if the activity MADS-box transcription factor is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 91 to 939-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a MADS-box transcription factor, or if the activity of the polypeptide At5g60440, preferably represented by SEQ ID NO. 102900, or a homolog or fragment thereof, or if the activity of a polypeptide encoded by a nucleic acid molecule comprising the nucleic acid SEQ ID NO. 102899, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 102899 or polypeptide SEQ ID NO. 102900, respectively, or a homolog or a fragment thereof, is increased or generated, or if the activity MADS-box transcription factor is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 93 to 149-percent is conferred as compared to a corresponding non-transformed wild type non-human organism.

Accordingly, in one embodiment, an increase of saccharic acid in a non-human organism, as compared to a corresponding non-transformed wild type non-human organism, is conferred in the process of the invention, if the activity of a polypeptide showing the activity of a MADS-box transcription factor, or if the activity of the polypeptide At5g60440, preferably represented by SEQ ID NO. 102900, or a homolog or fragment thereof, or if the activity of a polypeptide encoded by a nucleic acid molecule comprising the nucleic acid SEQ ID NO. 102899, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 102899 or polypeptide SEQ ID NO. 102900, respectively, or a homolog or a fragment thereof, is increased or generated, or if the activity MADS-box transcription factor is increased or generated in a non-human organism, like a microorganism or a plant cell, plant or part thereof, especially with non-targeted localization, whereby the respective line disclose in column 7 the fine chemical saccharic acid. For example, an increase of the saccharic acid of at least 1 percent, partitularly in a range of 43 to 64-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a protein kinase, or if the activity of the polypeptide At5g66710, preferably represented by SEQ ID NO. 122804, or a homolog or fragment thereof, or if the activity of a polypeptide encoded by a nucleic acid molecule comprising the nucleic acid SEQ ID NO. 122803, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 122803 or polypeptide SEQ ID NO. 122804, respectively, or a homolog or a fragment thereof, is increased or generated, or if the activity protein kinase is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 20 to 49-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a glycosyl transferase, or if the activity of the polypeptide AvinDRAFT_{—}1534, preferably represented by SEQ ID NO. 26121, or a homolog or fragment thereof, or if the activity of a polypeptide encoded by a nucleic acid molecule comprising the nucleic acid SEQ ID NO. 26120, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 26120 or polypeptide SEQ ID NO. 26121, respectively, or a homolog or a fragment thereof, is increased or generated, or if the activity glycosyl transferase is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 86 to 92-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a secindependent protein translocase, or if the activity of the polypeptide AvinDRAFT_{—}1624, preferably represented by SEQ ID NO. 26197, or a homolog or fragment thereof, or if the activity of a polypeptide encoded by a nucleic acid molecule comprising the nucleic acid SEQ ID NO. 26196, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 26196 or polypeptide SEQ ID NO. 26197, respectively, or a homolog or a fragment thereof, is increased or generated, or if the activity sec-independent protein translocase is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 26 to 41-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a beta-hydroxylase, or if the activity of the polypeptide AvinDRAFT_{—}2091, preferably represented by SEQ ID NO. 6041, or a homolog or fragment thereof, or if the activity of a polypeptide encoded by a nucleic acid molecule comprising the nucleic acid SEQ ID NO. 6040, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 6040 or polypeptide SEQ ID NO. 6041, respectively, or a homolog or a fragment thereof, is increased or generated, or if the activity beta-hydroxylase is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 43 to 115-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a elongation factor Tu, or if the activity of the polypeptide AvinDRAFT_{—}2344, preferably represented by SEQ ID NO. 27022, or a homolog or fragment thereof, or if the activity of a polypeptide encoded by a nucleic acid molecule comprising the nucleic acid SEQ ID NO. 27021, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 27021 or polypeptide SEQ ID NO. 27022, respectively, or a homolog or a fragment thereof, is increased or generated, or if the activity elongation factor Tu is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 131 to 486-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a elongation factor Tu, or if the activity of the polypeptide AvinDRAFT_{—}2344, preferably represented by SEQ ID NO. 27022, or a homolog or fragment thereof, or if the activity of a polypeptide encoded by a nucleic acid molecule comprising the nucleic acid SEQ ID NO. 27021, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 27021 or polypeptide SEQ ID NO. 27022, respectively, or a homolog or a fragment thereof, is increased or generated, or if the activity elongation factor Tu is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 58 to 207-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a ABC transporter permease protein, or if the activity of the polypeptide AvinDRAFT_{—}2521, preferably represented by SEQ ID NO. 27883, or a homolog or fragment thereof, or if the activity of a polypeptide encoded by a nucleic acid molecule comprising the nucleic acid SEQ ID NO. 27882, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 27882 or polypeptide SEQ ID NO. 27883, respectively, or a homolog or a fragment thereof, is increased or generated, or if the activity ABC transporter permease protein is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 77 to 744-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a ABC transporter permease protein, or if the activity of the polypeptide AvinDRAFT_{—}2521, preferably represented by SEQ ID NO. 27883, or a homolog or fragment thereof, or if the activity of a polypeptide encoded by a nucleic acid molecule comprising the nucleic acid SEQ ID NO. 27882, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 27882 or polypeptide SEQ ID NO. 27883, respectively, or a homolog or a fragment thereof, is increased or generated, or if the activity ABC transporter permease protein is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 24 to 95-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a acyl-CoA synthase, or if the activity of the polypeptide AvinDRAFT_{—}2754, preferably represented by SEQ ID NO. 28041, or a homolog or fragment thereof, or if the activity of a polypeptide encoded by a nucleic acid molecule comprising the nucleic acid SEQ ID NO. 28040, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 28040 or polypeptide SEQ ID NO. 28041, respectively, or a homolog or a fragment thereof, is increased or generated, or if the activity acyl-CoA synthase is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 28 to 54-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a 2-oxoglutarate dehydrogenase E1 subunit, or if the activity of the polypeptide AvinDRAFT_{—}3028, preferably represented by SEQ ID NO. 6076, or a homolog or fragment thereof, or if the activity of a polypeptide encoded by a nucleic acid molecule comprising the nucleic acid SEQ ID NO. 6075, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 6075 or polypeptide SEQ ID NO. 6076, respectively, or a homolog or a fragment thereof, is increased or generated, or if the activity 2-oxoglutarate dehydrogenase E1 subunit is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 77 to 249-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a 2-oxoglutarate dehydrogenase E1 subunit, or if the activity of the polypeptide AvinDRAFT_{—}3028, preferably represented by SEQ ID NO. 6076, or a homolog or fragment thereof, or if the activity of a polypeptide encoded by a nucleic acid molecule comprising the nucleic acid SEQ ID NO. 6075, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 6075 or polypeptide SEQ ID NO. 6076, respectively, or a homolog or a fragment thereof, is increased or generated, or if the activity 2-oxoglutarate dehydrogenase E1 subunit is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 19 to 137-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a 2-oxoglutarate dehydrogenase E1 subunit, or if the activity of the polypeptide AvinDRAFT_{—}3028, preferably represented by SEQ ID NO. 6076, or a homolog or fragment thereof, or if the activity of a polypeptide encoded by a nucleic acid molecule comprising the nucleic acid SEQ ID NO. 6075, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 6075 or polypeptide SEQ ID NO. 6076, respectively, or a homolog or a fragment thereof, is increased or generated, or if the activity 2-oxoglutarate dehydrogenase E1 subunit is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 39 to 273-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a 2-oxoglutarate dehydrogenase E1 subunit, or if the activity of the polypeptide AvinDRAFT_{—}3028, preferably represented by SEQ ID NO. 6076, or a homolog or fragment thereof, or if the activity of a polypeptide encoded by a nucleic acid molecule comprising the nucleic acid SEQ ID NO. 6075, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 6075 or polypeptide SEQ ID NO. 6076, respectively, or a homolog or a fragment thereof, is increased or generated, or if the activity 2-oxoglutarate dehydrogenase E1 subunit is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 65 to 493-percent is conferred as compared to a corresponding non-transformed wild type non-human organism.

Accordingly, in one embodiment, an increase of sedoheptulose-7-phosphate in a non-human organism, as compared to a corresponding non-transformed wild type non-human organism, is conferred in the process of the invention, if the activity of a polypeptide showing the activity of a 2-oxoglutarate dehydrogenase E1 subunit, or if the activity of the polypeptide AvinDRAFT_{—}3028, preferably represented by SEQ ID NO. 6076, or a homolog or fragment thereof, or if the activity of a polypeptide encoded by a nucleic acid molecule comprising the nucleic acid SEQ ID NO. 6075, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 6075 or polypeptide SEQ ID NO. 6076, respectively, or a homolog or a fragment thereof, is increased or generated, or if the activity 2-oxoglutarate dehydrogenase E1 subunit is increased or generated in a non-human organism, like a microorganism or a plant cell, plant or part thereof, especially with non-targeted localization, whereby the respective line disclose in column 7 the fine chemical sedoheptulose-7-phosphate. For example, an increase of the sedoheptulose-7-phosphate of at least 1 percent, particularly in a range of 50 to 123-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a GDP-mannose dehydrogenase, or if the activity of the polypeptide AvinDRAFT_{—}3135, preferably represented by SEQ ID NO. 72347, or a homolog or fragment thereof, or if the activity of a polypeptide encoded by a nucleic acid molecule comprising the nucleic acid SEQ ID NO. 72346, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 72346 or polypeptide SEQ ID NO. 72347, respectively, or a homolog or a fragment thereof, is increased or generated, or if the activity GDP-mannose dehydrogenase is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 427 to 3237-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a transcriptional regulator, or if the activity of the polypeptide AvinDRAFT_{—}3209, preferably represented by SEQ ID NO. 29287, or a homolog or fragment thereof, or if the activity of a polypeptide encoded by a nucleic acid molecule comprising the nucleic acid SEQ ID NO. 29286, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 29286 or polypeptide SEQ ID NO. 29287, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 78 to 155-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a transcriptional regulator, or if the activity of the polypeptide AvinDRAFT_{—}3209, preferably represented by SEQ ID NO. 29287, or a homolog or fragment thereof, or if the activity of a polypeptide encoded by a nucleic acid molecule comprising the nucleic acid SEQ ID NO. 29286, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 29286 or polypeptide SEQ ID NO. 29287, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 72 to 167-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a transcriptional regulator, or if the activity of the polypeptide AvinDRAFT_{—}3209, preferably represented by SEQ ID NO. 29287, or a homolog or fragment thereof, or if the activity of a polypeptide encoded by a nucleic acid molecule comprising the nucleic acid SEQ ID NO. 29286, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 29286 or polypeptide SEQ ID NO. 29287, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 20 to 26-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a oxidoreductase, or if the activity of the polypeptide AvinDRAFT_{—}3253, preferably represented by SEQ ID NO. 29501, or a homolog or fragment thereof, or if the activity of a polypeptide encoded by a nucleic acid molecule comprising the nucleic acid SEQ ID NO. 29500, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 29500 or polypeptide SEQ ID NO. 29501, respectively, or a homolog or a fragment thereof, is increased or generated, or if the activity oxidoreductase is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 19 to 61-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a ThiF family protein, or if the activity of the polypeptide AvinDRAFT_{—}3577, preferably represented by SEQ ID NO. 111156, or a homolog or fragment thereof, or if the activity of a polypeptide encoded by a nucleic acid molecule comprising the nucleic acid SEQ ID NO. 111155, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 111155 or polypeptide SEQ ID NO. 111156, respectively, or a homolog or a fragment thereof, is increased or generated, or if the activity ThiF family protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 27 to 74-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a CTP synthase, or if the activity of the polypeptide AvinDRAFT_{—}3605, preferably represented by SEQ ID NO. 31027, or a homolog or fragment thereof, or if the activity of a polypeptide encoded by a nucleic acid molecule comprising the nucleic acid SEQ ID NO. 31026, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 31026 or polypeptide SEQ ID NO. 31027, respectively, or a homolog or a fragment thereof, is increased or generated, or if the activity CTP synthase is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 170 to 743-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a CTP synthase, or if the activity of the polypeptide AvinDRAFT_{—}3605, preferably represented by SEQ ID NO. 31027, or a homolog or fragment thereof, or if the activity of a polypeptide encoded by a nucleic acid molecule comprising the nucleic acid SEQ ID NO. 31026, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 31026 or polypeptide SEQ ID NO. 31027, respectively, or a homolog or a fragment thereof, is increased or generated, or if the activity CTP synthase is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 67 to 683-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a CTP synthase, or if the activity of the polypeptide AvinDRAFT_{—}3605, preferably represented by SEQ ID NO. 31027, or a homolog or fragment thereof, or if the activity of a polypeptide encoded by a nucleic acid molecule comprising the nucleic acid SEQ ID NO. 31026, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 31026 or polypeptide SEQ ID NO. 31027, respectively, or a homolog or a fragment thereof, is increased or generated, or if the activity CTP synthase is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 46 to 102-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a protein-L-isoaspartate O-methyltransferase, or if the activity of the polypeptide AvinDRAFT_{—}3616, preferably represented by SEQ ID NO. 143122, or a homolog or fragment thereof, or if the activity of a polypeptide encoded by a nucleic acid molecule comprising the nucleic acid SEQ ID NO. 143121, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 143121 or polypeptide SEQ ID NO. 143122, respectively, or a homolog or a fragment thereof, is increased or generated, or if the activity protein-L-isoaspartate O-methyltransferase is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 55 to 99-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a recombinase A, or if the activity of the polypeptide AvinDRAFT_{—}3629, preferably represented by SEQ ID NO. 114232, or a homolog or fragment thereof, or if the activity of a polypeptide encoded by a nucleic acid molecule comprising the nucleic acid SEQ ID NO. 114231, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 114231 or polypeptide SEQ ID NO. 114232, respectively, or a homolog or a fragment thereof, is increased or generated, or if the activity recombinase A is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 44 to 288-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a recombinase A, or if the activity of the polypeptide AvinDRAFT_{—}3629, preferably represented by SEQ ID NO. 114232, or a homolog or fragment thereof, or if the activity of a polypeptide encoded by a nucleic acid molecule comprising the nucleic acid SEQ ID NO. 114231, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 114231 or polypeptide SEQ ID NO. 114232, respectively, or a homolog or a fragment thereof, is increased or generated, or if the activity recombinase A is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 107 to 400-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a acetyltransferase, or if the activity of the polypeptide AvinDRAFT_{—}4265, preferably represented by SEQ ID NO. 143399, or a homolog or fragment thereof, or if the activity of a polypeptide encoded by a nucleic acid molecule comprising the nucleic acid SEQ ID NO. 143398, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 143398 or polypeptide SEQ ID NO. 143399, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 29 to 72-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a acetyltransferase, or if the activity of the polypeptide AvinDRAFT_{—}4265, preferably represented by SEQ ID NO. 143399, or a homolog or fragment thereof, or if the activity of a polypeptide encoded by a nucleic acid molecule comprising the nucleic acid SEQ ID NO. 143398, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 143398 or polypeptide SEQ ID NO. 143399, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 62 to 126-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a enoyl-CoA hydratase, or if the activity of the polypeptide AvinDRAFT_{—}4420, preferably represented by SEQ ID NO. 73039, or a homolog or fragment thereof, or if the activity of a polypeptide encoded by a nucleic acid molecule comprising the nucleic acid SEQ ID NO. 73038, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 73038 or polypeptide SEQ ID NO. 73039, respectively, or a homolog or a fragment thereof, is increased or generated, or if the activity enoyl-CoA hydratase is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 74 to 180-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a NADH-quinone oxidoreductase subunit, or if the activity of the polypeptide AvinDRAFT_{—}4606, preferably represented by SEQ ID NO. 73720, or a homolog or fragment thereof, or if the activity of a polypeptide encoded by a nucleic acid molecule comprising the nucleic acid SEQ ID NO. 73719, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 73719 or polypeptide SEQ ID NO. 73720, respectively, or a homolog or a fragment thereof, is increased or generated, or if the activity NADH-quinone oxidoreductase subunit is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 125 to 130-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a NADH-quinone oxidoreductase subunit, or if the activity of the polypeptide AvinDRAFT_{—}4606, preferably represented by SEQ ID NO. 73720, or a homolog or fragment thereof, or if the activity of a polypeptide encoded by a nucleic acid molecule comprising the nucleic acid SEQ ID NO. 73719, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 73719 or polypeptide SEQ ID NO. 73720, respectively, or a homolog or a fragment thereof, is increased or generated, or if the activity NADH-quinone oxidoreductase subunit is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 24 to 34-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a NADH-quinone oxidoreductase subunit, or if the activity of the polypeptide AvinDRAFT_{—}4606, preferably represented by SEQ ID NO. 73720, or a homolog or fragment thereof, or if the activity of a polypeptide encoded by a nucleic acid molecule comprising the nucleic acid SEQ ID NO. 73719, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 73719 or polypeptide SEQ ID NO. 73720, respectively, or a homolog or a fragment thereof, is increased or generated, or if the activity NADH-quinone oxidoreductase subunit is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 28 to 74-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a isopropylmalate isomerase large subunit, or if the activity of the polypeptide AvinDRAFT_{—}4847, preferably represented by SEQ ID NO. 102942, or a homolog or fragment thereof, or if the activity of a polypeptide encoded by a nucleic acid molecule comprising the nucleic acid SEQ ID NO. 102941, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 102941 or polypeptide SEQ ID NO. 102942, respectively, or a homolog or a fragment thereof, is increased or generated, or if the activity isopropylmalate isomerase large subunit is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 44 to 86-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a hydrolase, or if the activity of the polypeptide AvinDRAFT_{—}5103, preferably represented by SEQ ID NO. 6511, or a homolog or fragment thereof, or if the activity of a polypeptide encoded by a nucleic acid molecule comprising the nucleic acid SEQ ID NO. 6510, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 6510 or polypeptide SEQ ID NO. 6511, respectively, or a homolog or a fragment thereof, is increased or generated, or if the activity hydrolase is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 83 to 1502-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a hydrolase, or if the activity of the polypeptide AvinDRAFT_{—}5103, preferably represented by SEQ ID NO. 6511, or a homolog or fragment thereof, or if the activity of a polypeptide encoded by a nucleic acid molecule comprising the nucleic acid SEQ ID NO. 6510, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 6510 or polypeptide SEQ ID NO. 6511, respectively, or a homolog or a fragment thereof, is increased or generated, or if the activity hydrolase is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 62 to 605-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a hydrolase, or if the activity of the polypeptide AvinDRAFT_{—}5103, preferably represented by SEQ ID NO. 6511, or a homolog or fragment thereof, or if the activity of a polypeptide encoded by a nucleic acid molecule comprising the nucleic acid SEQ ID NO. 6510, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 6510 or polypeptide SEQ ID NO. 6511, respectively, or a homolog or a fragment thereof, is increased or generated, or if the activity hydrolase is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 31 to 48-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a glutamate-ammonia-ligase, or if the activity of the polypeptide AvinDRAFT_{—}5246, preferably represented by SEQ ID NO. 32038, or a homolog or fragment thereof, or if the activity of a polypeptide encoded by a nucleic acid molecule comprising the nucleic acid SEQ ID NO. 32037, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 32037 or polypeptide SEQ ID NO. 32038, respectively, or a homolog or a fragment thereof, is increased or generated, or if the activity glutamate-ammonia-ligase is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 82 to 370-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a glutamate-ammonia-ligase, or if the activity of the polypeptide AvinDRAFT_{—}5246, preferably represented by SEQ ID NO. 32038, or a homolog or fragment thereof, or if the activity of a polypeptide encoded by a nucleic acid molecule comprising the nucleic acid SEQ ID NO. 32037, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 32037 or polypeptide SEQ ID NO. 32038, respectively, or a homolog or a fragment thereof, is increased or generated, or if the activity glutamate-ammonia-ligase is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 61 to 200-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a glutamate-ammonia-ligase, or if the activity of the polypeptide AvinDRAFT_{—}5246, preferably represented by SEQ ID NO. 32038, or a homolog or fragment thereof, or if the activity of a polypeptide encoded by a nucleic acid molecule comprising the nucleic acid SEQ ID NO. 32037, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 32037 or polypeptide SEQ ID NO. 32038, respectively, or a homolog or a fragment thereof, is increased or generated, or if the activity glutamate-ammonia-ligase is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 27 to 38-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a fumarylacetoacetate hydrolase, or if the activity of the polypeptide AvinDRAFT_{—}5292, preferably represented by SEQ ID NO. 32309, or a homolog or fragment thereof, or if the activity of a polypeptide encoded by a nucleic acid molecule comprising the nucleic acid SEQ ID NO. 32308, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 32308 or polypeptide SEQ ID NO. 32309, respectively, or a homolog or a fragment thereof, is increased or generated, or if the activity fumarylacetoacetate hydrolase is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 186 to 188-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a glycosyltransferase, or if the activity of the polypeptide AvinDRAFT_{—}5967, preferably represented by SEQ ID NO. 143496, or a homolog or fragment thereof, or if the activity of a polypeptide encoded by a nucleic acid molecule comprising the nucleic acid SEQ ID NO. 143495, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 143495 or polypeptide SEQ ID NO. 143496, respectively, or a homolog or a fragment thereof, is increased or generated, or if the activity glycosyltransferase is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 1093 to 3766-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a peptidylprolyl cis-trans isomerase, or if the activity of the polypeptide AvinDRAFT_{—}6075, preferably represented by SEQ ID NO. 6675, or a homolog or fragment thereof, or if the activity of a polypeptide encoded by a nucleic acid molecule comprising the nucleic acid SEQ ID NO. 6674, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 6674 or polypeptide SEQ ID NO. 6675, respectively, or a homolog or a fragment thereof, is increased or generated, or if the activity peptidyl-prolyl cis-trans isomerase is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 31 to 126-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a NAD(P)H dehydrogenase (quinone), or if the activity of the polypeptide AVINDRAFT_{—}6261, preferably represented by SEQ ID NO. 143615, or a homolog or fragment thereof, or if the activity of a polypeptide encoded by a nucleic acid molecule comprising the nucleic acid SEQ ID NO. 143614, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 143614 or polypeptide SEQ ID NO. 143615, respectively, or a homolog or a fragment thereof, is increased or generated, or if the activity NAD(P)H dehydrogenase (quinone) is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 33 to 77-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a electron transfer flavoprotein subunit beta, or if the activity of the polypeptide AvinDRAFT_{—}6679, preferably represented by SEQ ID NO. 74730, or a homolog or fragment thereof, or if the activity of a polypeptide encoded by a nucleic acid molecule comprising the nucleic acid SEQ ID NO. 74729, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 74729 or polypeptide SEQ ID NO. 74730, respectively, or a homolog or a fragment thereof, is increased or generated, or if the activity electron transfer flavoprotein subunit beta is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 117 to 1104-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a electron transfer flavoprotein subunit beta, or if the activity of the polypeptide AvinDRAFT_{—}6679, preferably represented by SEQ ID NO. 74730, or a homolog or fragment thereof, or if the activity of a polypeptide encoded by a nucleic acid molecule comprising the nucleic acid SEQ ID NO. 74729, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 74729 or polypeptide SEQ ID NO. 74730, respectively, or a homolog or a fragment thereof, is increased or generated, or if the activity electron transfer flavoprotein subunit beta is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 80 to 613-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a purine nucleoside phosphorylase, or if the activity of the polypeptide AvinDRAFT_{—}6700, preferably represented by SEQ ID NO. 34045, or a homolog or fragment thereof, or if the activity of a polypeptide encoded by a nucleic acid molecule comprising the nucleic acid SEQ ID NO. 34044, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 34044 or polypeptide SEQ ID NO. 34045, respectively, or a homolog or a fragment thereof, is increased or generated, or if the activity purine nucleoside phosphorylase is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 53 to 114-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a purine nucleoside phosphorylase, or if the activity of the polypeptide AvinDRAFT_{—}6700, preferably represented by SEQ ID NO. 34045, or a homolog or fragment thereof, or if the activity of a polypeptide encoded by a nucleic acid molecule comprising the nucleic acid SEQ ID NO. 34044, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 34044 or polypeptide SEQ ID NO. 34045, respectively, or a homolog or a fragment thereof, is increased or generated, or if the activity purine nucleoside phosphorylase is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 60 to 108-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a transcriptional regulator protein, or if the activity of the polypeptide AvinDRAFT_{—}6864, preferably represented by SEQ ID NO. 34205, or a homolog or fragment thereof, or if the activity of a polypeptide encoded by a nucleic acid molecule comprising the nucleic acid SEQ ID NO. 34204, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 34204 or polypeptide SEQ ID NO. 34205, respectively, or a homolog or a fragment thereof, is increased or generated, or if the activity transcriptional regulator protein is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 62 to 341-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a AX653549-protein, or if the activity of the polypeptide AX653549, preferably represented by SEQ ID NO. 34302, or a homolog or fragment thereof, or if the activity of a polypeptide encoded by a nucleic acid molecule comprising the nucleic acid SEQ ID NO. 34301, preferably the coding region thereof, or a homolog or fragment thereof, e.g. derived from Oryza sativa, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 34301 or polypeptide SEQ ID NO. 34302, respectively, or a homolog or a fragment thereof, is increased or generated, or if the activity AX653549-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 28 to 50-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a AY087308-protein, or if the activity of the polypeptide AY087308, preferably represented by SEQ ID NO. 34603, or a homolog or fragment thereof, or if the activity of a polypeptide encoded by a nucleic acid molecule comprising the nucleic acid SEQ ID NO. 34602, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 34602 or polypeptide SEQ ID NO. 34603, respectively, or a homolog or a fragment thereof, is increased or generated, or if the activity AY087308-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 28 to 47-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a AY087308-protein, or if the activity of the polypeptide AY087308, preferably represented by SEQ ID NO. 34603, or a homolog or fragment thereof, or if the activity of a polypeptide encoded by a nucleic acid molecule comprising the nucleic acid SEQ ID NO. 34602, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 34602 or polypeptide SEQ ID NO. 34603, respectively, or a homolog or a fragment thereof, is increased or generated, or if the activity AY087308-protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 26 to 85-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a pyruvate kinase, or if the activity of the polypeptide AY623894, preferably represented by SEQ ID NO. 103434, or a homolog or fragment thereof, or if the activity of a polypeptide encoded by a nucleic acid molecule comprising the nucleic acid SEQ ID NO. 103433, preferably the coding region thereof, or a homolog or fragment thereof, e.g. derived from Zea mays, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 103433 or polypeptide SEQ ID NO. 103434, respectively, or a homolog or a fragment thereof, is increased or generated, or if the activity pyruvate kinase is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 58 to 113-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a pyruvate kinase, or if the activity of the polypeptide AY623894, preferably represented by SEQ ID NO. 103434, or a homolog or fragment thereof, or if the activity of a polypeptide encoded by a nucleic acid molecule comprising the nucleic acid SEQ ID NO. 103433, preferably the coding region thereof, or a homolog or fragment thereof, e.g. derived from Zea mays, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 103433 or polypeptide SEQ ID NO. 103434, respectively, or a homolog or a fragment thereof, is increased or generated, or if the activity pyruvate kinase is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 33 to 167-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a threonine synthase, or if the activity of the polypeptide B0004, preferably represented by SEQ ID NO. 34890, or a homolog or fragment thereof, or if the activity of a polypeptide encoded by a nucleic acid molecule comprising the nucleic acid SEQ ID NO. 34889, preferably the coding region thereof, or a homolog or fragment thereof, e.g. derived from Escherichia coli, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 34889 or polypeptide SEQ ID NO. 34890, respectively, or a homolog or a fragment thereof, is increased or generated, or if the activity threonine synthase is increased or generated in a non-human organism, like a microorganism or a plant cell, plant or part thereof, especially with plastidic localization, whereby the respective line disclose in column 7 the fine chemical glucose. For example, an increase of the glucose of at least 1 percent, particularly in a range of 62 to 423-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a threonine synthase, or if the activity of the polypeptide B0004, preferably represented by SEQ ID NO. 34890, or a homolog or fragment thereof, or if the activity of a polypeptide encoded by a nucleic acid molecule comprising the nucleic acid SEQ ID NO. 34889, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 34889 or polypeptide SEQ ID NO. 34890, respectively, or a homolog or a fragment thereof, is increased or generated, or if the activity threonine synthase is increased or generated in a non-human organism, like a microorganism or a plant cell, plant or part thereof, especially with plastidic localization, whereby the respective line disclose in column 7 the fine chemical sucrose. For example, an increase of the sucrose of at least 1 percent, particularly in a range of 25 to 120-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a b0050-protein, or if the activity of the polypeptide B0050, preferably represented by SEQ ID NO. 103959, or a homolog or fragment thereof, or if the activity of a polypeptide encoded by a nucleic acid molecule comprising the nucleic acid SEQ ID NO. 103958, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 103958 or polypeptide SEQ ID NO. 103959, respectively, or a homolog or a fragment thereof, is increased or generated, or if the activity b0050-protein is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 33 to 63-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a L-ribulose-5-phosphate 4-epimerase, or if the activity of the polypeptide B0061, preferably represented by SEQ ID NO. 35205, or a homolog or fragment thereof, or if the activity of a polypeptide encoded by a nucleic acid molecule comprising the nucleic acid SEQ ID NO. 35204, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 35204 or polypeptide SEQ ID NO. 35205, respectively, or a homolog or a fragment thereof, is increased or generated, or if the activity L-ribulose-5-phosphate 4-epimerase is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 38 to 63-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a glucose dehydrogenase, or if the activity of the polypeptide B0124, preferably represented by SEQ ID NO. 35483, or a homolog or fragment thereof, or if the activity of a polypeptide encoded by a nucleic acid molecule comprising the nucleic acid SEQ ID NO. 35482, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 35482 or polypeptide SEQ ID NO. 35483, respectively, or a homolog or a fragment thereof, is increased or generated, or if the activity glucose dehydrogenase is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 27 to 83-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a serine protease, or if the activity of the polypeptide B0161, preferably represented by SEQ ID NO. 7082, or a homolog or fragment thereof, or if the activity of a polypeptide encoded by a nucleic acid molecule comprising the nucleic acid SEQ ID NO. 7081, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 7081 or polypeptide SEQ ID NO. 7082, respectively, or a homolog or a fragment thereof, is increased or generated, or if the activity serine protease is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 24 to 53-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a serine protease, or if the activity of the polypeptide B0161, preferably represented by SEQ ID NO. 7082, or a homolog or fragment thereof, or if the activity of a polypeptide encoded by a nucleic acid molecule comprising the nucleic acid SEQ ID NO. 7081, preferably the coding region thereof, or a homolog or fragment thereof, e.g. derived from Escherichia coli, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 7081 or polypeptide SEQ ID NO. 7082, respectively, or a homolog or a fragment thereof, is increased or generated, or if the activity serine protease is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 25 to 62-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a acyl-CoA dehydrogenase, or if the activity of the polypeptide B0221, preferably represented by SEQ ID NO. 35591, or a homolog or fragment thereof, or if the activity of a polypeptide encoded by a nucleic acid molecule comprising the nucleic acid SEQ ID NO. 35590, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 35590 or polypeptide SEQ ID NO. 35591, respectively, or a homolog or a fragment thereof, is increased or generated, or if the activity acyl-CoA dehydrogenase is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 25 to 89-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a b0251-protein, or if the activity of the polypeptide B0251, preferably represented by SEQ ID NO. 143743, or a homolog or fragment thereof, or if the activity of a polypeptide encoded by a nucleic acid molecule comprising the nucleic acid SEQ ID NO. 143742, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 143742 or polypeptide SEQ ID NO. 143743, respectively, or a homolog or a fragment thereof, is increased or generated, or if the activity b0251-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 20 to 59-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a betaine aldehyde dehydrogenase, or if the activity of the polypeptide B0312, preferably represented by SEQ ID NO. 121112, or a homolog or fragment thereof, or if the activity of a polypeptide encoded by a nucleic acid molecule comprising the nucleic acid SEQ ID NO. 121111, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 121111 or polypeptide SEQ ID NO. 121112, respectively, or a homolog or a fragment thereof, is increased or generated, or if the activity betaine aldehyde dehydrogenase is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 76 to 386-percent is conferred as compared to a corresponding non-transformed wild type non-human organism.

Accordingly, in one embodiment, an increase of melibiose in a non-human organism, as compared to a corresponding non-transformed wild type non-human organism, is conferred in the process of the invention, if the activity of a polypeptide showing the activity of a beta-galactosidase, or if the activity of the polypeptide B0344, preferably represented by SEQ ID NO. 35734, or a homolog or fragment thereof, or if the activity of a polypeptide encoded by a nucleic acid molecule comprising the nucleic acid SEQ ID NO. 35733, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 35733 or polypeptide SEQ ID NO. 35734, respectively, or a homolog or a fragment thereof, is increased or generated, or if the activity beta-galactosidase is increased or generated in a non-human organism, like a microorganism or a plant cell, plant or part thereof, especially with plastidic localization, whereby the respective line disclose in column 7 the fine chemical melibiose. For example, an increase of the melibiose of at least 1 percent, particularly in a range of 65 to 79-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a b0362-protein, or if the activity of the polypeptide B0362, preferably represented by SEQ ID NO. 143762, or a homolog or fragment thereof, or if the activity of a polypeptide encoded by a nucleic acid molecule comprising the nucleic acid SEQ ID NO. 143761, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 143761 or polypeptide SEQ ID NO. 143762, respectively, or a homolog or a fragment thereof, is increased or generated, or if the activity b0362-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 18 to 22-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a penicillin-binding protein, or if the activity of the polypeptide B0376, preferably represented by SEQ ID NO. 143772, or a homolog or fragment thereof, or if the activity of a polypeptide encoded by a nucleic acid molecule comprising the nucleic acid SEQ ID NO. 143771, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 143771 or polypeptide SEQ ID NO. 143772, respectively, or a homolog or a fragment thereof, is increased or generated, or if the activity penicillin-binding protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 27 to 37-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a ATP-binding component of a transport system, or if the activity of the polypeptide B0449, preferably represented by SEQ ID NO. 7334, or a homolog or fragment thereof, or if the activity of a polypeptide encoded by a nucleic acid molecule comprising the nucleic acid SEQ ID NO. 7333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 7333 or polypeptide SEQ ID NO. 7334, respectively, or a homolog or a fragment thereof, is increased or generated, or if the activity ATP-binding component of a transport system is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 121 to 729-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a ATP-binding component of a transport system, or if the activity of the polypeptide B0449, preferably represented by SEQ ID NO. 7334, or a homolog or fragment thereof, or if the activity of a polypeptide encoded by a nucleic acid molecule comprising the nucleic acid SEQ ID NO. 7333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 7333 or polypeptide SEQ ID NO. 7334, respectively, or a homolog or a fragment thereof, is increased or generated, or if the activity ATP-binding component of a transport system is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 63 to 243-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a membrane transport protein, or if the activity of the polypeptide B0486, preferably represented by SEQ ID NO. 7687, or a homolog or fragment thereof, or if the activity of a polypeptide encoded by a nucleic acid molecule comprising the nucleic acid SEQ ID NO. 7686, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 7686 or polypeptide SEQ ID NO. 7687, respectively, or a homolog or a fragment thereof, is increased or generated, or if the activity membrane transport protein is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 61 to 135-percent is conferred as compared to a corresponding non-transformed wild type non-human organism.

Accordingly, in one embodiment, an increase of saccharic acid in a non-human organism, as compared to a corresponding non-transformed wild type non-human organism, is conferred in the process of the invention, if the activity of a polypeptide showing the activity of a b0488-protein, or if the activity of the polypeptide B0488, preferably represented by SEQ ID NO. 143818, or a homolog or fragment thereof, or if the activity of a polypeptide encoded by a nucleic acid molecule comprising the nucleic acid SEQ ID NO. 143817, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 143817 or polypeptide SEQ ID NO. 143818, respectively, or a homolog or a fragment thereof, is increased or generated, or if the activity b0488-protein is increased or generated in a non-human organism, like a microorganism or a plant cell, plant or part thereof, especially with plastidic localization, whereby the respective line disclose in column 7 the fine chemical saccharic acid. For example, an increase of the saccharic acid of at least 1 percent, particularly in a range of 57 to 74-percent is conferred as compared to a corresponding non-transformed wild type non-human organism.

Accordingly, in one embodiment, an increase of galactinol in a non-human organism, as compared to a corresponding non-transformed wild type non-human organism, is conferred in the process of the invention, if the activity of a polypeptide showing the activity of a glycerate kinase, or if the activity of the polypeptide B0514, preferably represented by SEQ ID NO. 134403, or a homolog or fragment thereof, or if the activity of a polypeptide encoded by a nucleic acid molecule comprising the nucleic acid SEQ ID NO. 134402, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 134402 or polypeptide SEQ ID NO. 134403, respectively, or a homolog or a fragment thereof, is increased or generated, or if the activity glycerate kinase is increased or generated in a non-human organism, like a microorganism or a plant cell, plant or part thereof, especially with non-targeted localization, whereby the respective line disclose in column 7 the fine chemical galactinol. For example, an increase of the galactinol of at least 1 percent, particularly in a range of 42 to 174-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a isochorismate synthase, or if the activity of the polypeptide B0593, preferably represented by SEQ ID NO. 35968, or a homolog or fragment thereof, or if the activity of a polypeptide encoded by a nucleic acid molecule comprising the nucleic acid SEQ ID NO. 35967, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 35967 or polypeptide SEQ ID NO. 35968, respectively, or a homolog or a fragment thereof, is increased or generated, or if the activity isochorismate synthase is increased or generated in a non-human organism, like a microorganism or a plant cell, plant or part thereof, especially with plastidic localization, whereby the respective line disclose in column 7 the fine chemical fructose. For example, an increase of the fructose of at least 1 percent, particularly in a range of 188 to 824-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a isochorismate synthase, or if the activity of the polypeptide B0593, preferably represented by SEQ ID NO. 35968, or a homolog or fragment thereof, or if the activity of a polypeptide encoded by a nucleic acid molecule comprising the nucleic acid SEQ ID NO. 35967, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 35967 or polypeptide SEQ ID NO. 35968, respectively, or a homolog or a fragment thereof, is increased or generated, or if the activity isochorismate synthase is increased or generated in a non-human organism, like a microorganism or a plant cell, plant or part thereof, especially with plastidic localization, whereby the respective line disclose in column 7 the fine chemical glucose. For example, an increase of the glucose of at least 1 percent, particularly in a range of 73 to 258-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a isochorismate synthase, or if the activity of the polypeptide B0593, preferably represented by SEQ ID NO. 35968, or a homolog or fragment thereof, or if the activity of a polypeptide encoded by a nucleic acid molecule comprising the nucleic acid SEQ ID NO. 35967, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 35967 or polypeptide SEQ ID NO. 35968, respectively, or a homolog or a fragment thereof, is increased or generated, or if the activity isochorismate synthase is increased or generated in a non-human organism, like a microorganism or a plant cell, plant or part thereof, especially with plastidic localization, whereby the respective line disclose in column 7 the fine chemical raffinose. For example, an increase of the raffinose of at least 1 percent, particularly in a range of 70 to 127-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a isochorismate synthase, or if the activity of the polypeptide B0593, preferably represented by SEQ ID NO. 35968, or a homolog or fragment thereof, or if the activity of a polypeptide encoded by a nucleic acid molecule comprising the nucleic acid SEQ ID NO. 35967, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 35967 or polypeptide SEQ ID NO. 35968, respectively, or a homolog or a fragment thereof, is increased or generated, or if the activity isochorismate synthase is increased or generated in a non-human organism, like a microorganism or a plant cell, plant or part thereof, especially with plastidic localization, whereby the respective line disclose in column 7 the fine chemical sucrose. For example, an increase of the sucrose of at least 1 percent, particularly in a range of 25 to 48-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a B0644-protein, or if the activity of the polypeptide B0644, preferably represented by SEQ ID NO. 143902, or a homolog or fragment thereof, or if the activity of a polypeptide encoded by a nucleic acid molecule comprising the nucleic acid SEQ ID NO. 143901, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 143901 or polypeptide SEQ ID NO. 143902, respectively, or a homolog or a fragment thereof, is increased or generated, or if the activity B0644-protein is increased or generated in a non-human organism, like a microorganism or a plant cell, plant or part thereof, especially with plastidic localization, whereby the respective line disclose in column 7 the fine chemical 3,4,5-trihydroxypentanoic acid. For example, an increase of the 3,4,5-trihydroxypentanoic acid of at least 1 percent, particularly in a range of 40 to 79-percent is conferred as compared to a corresponding non-transformed wild type non-human organism.

Accordingly, in one embodiment, an increase of saccharic acid in a non-human organism, as compared to a corresponding non-transformed wild type non-human organism, is conferred in the process of the invention, if the activity of a polypeptide showing the activity of a pyrimidine-specific ribonucleoside hydrolase, or if the activity of the polypeptide B0651, preferably represented by SEQ ID NO. 143955, or a homolog or fragment thereof, or if the activity of a polypeptide encoded by a nucleic acid molecule comprising the nucleic acid SEQ ID NO. 143954, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 143954 or polypeptide SEQ ID NO. 143955, respectively, or a homolog or a fragment thereof, is increased or generated, or if the activity pyrimidine-specific ribonucleoside hydrolase is increased or generated in a non-human organism, like a microorganism or a plant cell, plant or part thereof, especially with non-targeted localization, whereby the respective line disclose in column 7 the fine chemical saccharic acid. For example, an increase of the saccharic acid of at least 1 percent, particularly in a range of 40 to 46-percent is conferred as compared to a corresponding non-transformed wild type non-human organism.

Accordingly, in one embodiment, an increase of erythrol in a non-human organism, as compared to a corresponding non-transformed wild type non-human organism, is conferred in the process of the invention, if the activity of a polypeptide showing the activity of a asparagine synthetase B, or if the activity of the polypeptide B0674, preferably represented by SEQ ID NO. 144208, or a homolog or fragment thereof, or if the activity of a polypeptide encoded by a nucleic acid molecule comprising the nucleic acid SEQ ID NO. 144207, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 144207 or polypeptide SEQ ID NO. 144208, respectively, or a homolog or a fragment thereof, is increased or generated, or if the activity asparagine synthetase B is increased or generated in a non-human organism, like a microorganism or a plant cell, plant or part thereof, especially with plastidic localization, whereby the respective line disclose in column 7 the fine chemical erythrol. For example, an increase of the erythrol of at least 1 percent, particularly in a range of 239 to 281-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a sensor histidine kinase, or if the activity of the polypeptide B0695, preferably represented by SEQ ID NO. 93679, or a homolog or fragment thereof, or if the activity of a polypeptide encoded by a nucleic acid molecule comprising the nucleic acid SEQ ID NO. 93678, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 93678 or polypeptide SEQ ID NO. 93679, respectively, or a homolog or a fragment thereof, is increased or generated, or if the activity sensor histidine kinase is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 27 to 58-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a succinyl-CoA synthetase beta chain, or if the activity of the polypeptide B0728, preferably represented by SEQ ID NO. 93794, or a homolog or fragment thereof, or if the activity of a polypeptide encoded by a nucleic acid molecule comprising the nucleic acid SEQ ID NO. 93793, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 93793 or polypeptide SEQ ID NO. 93794, respectively, or a homolog or a fragment thereof, is increased or generated, or if the activity succinyl-CoA synthetase beta chain is increased or generated in a non-human organism, like a microorganism or a plant cell, plant or part thereof, especially with plastidic localization, whereby the respective line disclose in column 7 the fine chemical myo-inositol. For example, an increase of the myo-inositol of at least 1 percent, particularly in a range of 26 to 47-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a zinc transporter, or if the activity of the polypeptide B0752, preferably represented by SEQ ID NO. 36115, or a homolog or fragment thereof, or if the activity of a polypeptide encoded by a nucleic acid molecule comprising the nucleic acid SEQ ID NO. 36114, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 36114 or polypeptide SEQ ID NO. 36115, respectively, or a homolog or a fragment thereof, is increased or generated, or if the activity zinc transporter is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 77 to 258-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a zinc transporter, or if the activity of the polypeptide B0752, preferably represented by SEQ ID NO. 36115, or a homolog or fragment thereof, or if the activity of a polypeptide encoded by a nucleic acid molecule comprising the nucleic acid SEQ ID NO. 36114, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 36114 or polypeptide SEQ ID NO. 36115, respectively, or a homolog or a fragment thereof, is increased or generated, or if the activity zinc transporter is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 76 to 641-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a zinc transporter, or if the activity of the polypeptide B0752, preferably represented by SEQ ID NO. 36115, or a homolog or fragment thereof, or if the activity of a polypeptide encoded by a nucleic acid molecule comprising the nucleic acid SEQ ID NO. 36114, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 36114 or polypeptide SEQ ID NO. 36115, respectively, or a homolog or a fragment thereof, is increased or generated, or if the activity zinc transporter is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 30 to 78-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a zinc transporter, or if the activity of the polypeptide B0752, preferably represented by SEQ ID NO. 36115, or a homolog or fragment thereof, or if the activity of a polypeptide encoded by a nucleic acid molecule comprising the nucleic acid SEQ ID NO. 36114, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 36114 or polypeptide SEQ ID NO. 36115, respectively, or a homolog or a fragment thereof, is increased or generated, or if the activity zinc transporter is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 65 to 646-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a b0801-protein, or if the activity of the polypeptide B0801, preferably represented by SEQ ID NO. 75808, or a homolog or fragment thereof, or if the activity of a polypeptide encoded by a nucleic acid molecule comprising the nucleic acid SEQ ID NO. 75807, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 75807 or polypeptide SEQ ID NO. 75808, respectively, or a homolog or a fragment thereof, is increased or generated, or if the activity b0801-protein is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 108 to 170-percent is conferred as compared to a corresponding non-transformed wild type non-human organism.

Accordingly, in one embodiment, an increase of sedoheptulose-7-phosphate in a non-human organism, as compared to a corresponding non-transformed wild type non-human organism, is conferred in the process of the invention, if the activity of a polypeptide showing the activity of a putrescine transport system permease protein, or if the activity of the polypeptide B0856, preferably represented by SEQ ID NO. 131360, or a homolog or fragment thereof, or if the activity of a polypeptide encoded by a nucleic acid molecule comprising the nucleic acid SEQ ID NO. 131359, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 131359 or polypeptide SEQ ID NO. 131360, respectively, or a homolog or a fragment thereof, is increased or generated, or if the activity putrescine transport system permease protein is increased or generated in a non-human organism, like a microorganism or a plant cell, plant or part thereof, especially with plastidic localization, whereby the respective line disclose in column 7 the fine chemical sedoheptulose-7-phosphate. For example, an increase of the sedoheptulose-7-phosphate of at least 1 percent, particularly in a range of 74 to 127-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a leucyl/phenylalanyl-tRNA-protein transferase, or if the activity of the polypeptide B0885, preferably represented by SEQ ID NO. 36490, or a homolog or fragment thereof, or if the activity of a polypeptide encoded by a nucleic acid molecule comprising the nucleic acid SEQ ID NO. 36489, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 36489 or polypeptide SEQ ID NO. 36490, respectively, or a homolog or a fragment thereof, is increased or generated, or if the activity leucyl/phenylalanyl-tRNA-protein transferase is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 67 to 76-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a b0970-protein, or if the activity of the polypeptide B0970, preferably represented by SEQ ID NO. 144480, or a homolog or fragment thereof, or if the activity of a polypeptide encoded by a nucleic acid molecule comprising the nucleic acid SEQ ID NO. 144479, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 144479 or polypeptide SEQ ID NO. 144480, respectively, or a homolog or a fragment thereof, is increased or generated, or if the activity b0970-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 22 to 130-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a phosphoanhydride phosphorylase, or if the activity of the polypeptide B0980, preferably represented by SEQ ID NO. 36810, or a homolog or fragment thereof, or if the activity of a polypeptide encoded by a nucleic acid molecule comprising the nucleic acid SEQ ID NO. 36809, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 36809 or polypeptide SEQ ID NO. 36810, respectively, or a homolog or a fragment thereof, is increased or generated, or if the activity phosphoanhydride phosphorylase is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 26 to 130-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a b1003-protein, or if the activity of the polypeptide B1003, preferably represented by SEQ ID NO. 7942, or a homolog or fragment thereof, or if the activity of a polypeptide encoded by a nucleic acid molecule comprising the nucleic acid SEQ ID NO. 7941, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 7941 or polypeptide SEQ ID NO. 7942, respectively, or a homolog or a fragment thereof, is increased or generated, or if the activity b1003-protein is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 278 to 1463-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a b1003-protein, or if the activity of the polypeptide B1003, preferably represented by SEQ ID NO. 7942, or a homolog or fragment thereof, or if the activity of a polypeptide encoded by a nucleic acid molecule comprising the nucleic acid SEQ ID NO. 7941, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 7941 or polypeptide SEQ ID NO. 7942, respectively, or a homolog or a fragment thereof, is increased or generated, or if the activity b1003-protein is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 44 to 339-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a b1003-protein, or if the activity of the polypeptide B1003, preferably represented by SEQ ID NO. 7942, or a homolog or fragment thereof, or if the activity of a polypeptide encoded by a nucleic acid molecule comprising the nucleic acid SEQ ID NO. 7941, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 7941 or polypeptide SEQ ID NO. 7942, respectively, or a homolog or a fragment thereof, is increased or generated, or if the activity b1003-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 30 to 102-percent is conferred as compared to a corresponding non-transformed wild type non-human organism.

Accordingly, in one embodiment, an increase of sedoheptulose-7-phosphate in a non-human organism, as compared to a corresponding non-transformed wild type non-human organism, is conferred in the process of the invention, if the activity of a polypeptide showing the activity of a b1003-protein, or if the activity of the polypeptide B1003, preferably represented by SEQ ID NO. 7942, or a homolog or fragment thereof, or if the activity of a polypeptide encoded by a nucleic acid molecule comprising the nucleic acid SEQ ID NO. 7941, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 7941 or polypeptide SEQ ID NO. 7942, respectively, or a homolog or a fragment thereof, is increased or generated, or if the activity b1003-protein is increased or generated in a non-human organism, like a microorganism or a plant cell, plant or part thereof, especially with non-targeted localization, whereby the respective line disclose in column 7 the fine chemical sedoheptulose-7-phosphate. For example, an increase of the sedoheptulose-7-phosphate of at least 1 percent, particularly in a range of 62 to 799-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a b1003-protein, or if the activity of the polypeptide B1003, preferably represented by SEQ ID NO. 7942, or a homolog or fragment thereof, or if the activity of a polypeptide encoded by a nucleic acid molecule comprising the nucleic acid SEQ ID NO. 7941, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 7941 or polypeptide SEQ ID NO. 7942, respectively, or a homolog or a fragment thereof, is increased or generated, or if the activity b1003-protein is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 57 to 120-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a b1046-protein, or if the activity of the polypeptide B1046, preferably represented by SEQ ID NO. 124052, or a homolog or fragment thereof, or if the activity of a polypeptide encoded by a nucleic acid molecule comprising the nucleic acid SEQ ID NO. 124051, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 124051 or polypeptide SEQ ID NO. 124052, respectively, or a homolog or a fragment thereof, is increased or generated, or if the activity b1046-protein is increased or generated in a non-human organism, like a microorganism or a plant cell, plant or part thereof, especially with plastidic localization, whereby the respective line disclose in column 7 the fine chemical 3,4,5-trihydroxypentanoic acid. For example, an increase of the 3,4,5-trihydroxypentanoic acid of at least 1 percent, particularly in a range of 33 to 49-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a multidrug resistance protein, or if the activity of the polypeptide B1065, preferably represented by SEQ ID NO. 76033, or a homolog or fragment thereof, or if the activity of a polypeptide encoded by a nucleic acid molecule comprising the nucleic acid SEQ ID NO. 76032, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 76032 or polypeptide SEQ ID NO. 76033, respectively, or a homolog or a fragment thereof, is increased or generated, or if the activity multidrug resistance protein is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 19 to 35-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a multidrug resistance protein, or if the activity of the polypeptide B1065, preferably represented by SEQ ID NO. 76033, or a homolog or fragment thereof, or if the activity of a polypeptide encoded by a nucleic acid molecule comprising the nucleic acid SEQ ID NO. 76032, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 76032 or polypeptide SEQ ID NO. 76033, respectively, or a homolog or a fragment thereof, is increased or generated, or if the activity multidrug resistance protein is increased or generated in a non-human organism, like a microorganism or a plant cell, plant or part thereof, especially with plastidic localization, whereby the respective line disclose in column 7 the fine chemical sucrose. For example, an increase of the sucrose of at least 1 percent, particularly in a range of 32 to 37-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a outer membrane receptor protein precursor, or if the activity of the polypeptide B1102, preferably represented by SEQ ID NO. 114980, or a homolog or fragment thereof, or if the activity of a polypeptide encoded by a nucleic acid molecule comprising the nucleic acid SEQ ID NO. 114979, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 114979 or polypeptide SEQ ID NO. 114980, respectively, or a homolog or a fragment thereof, is increased or generated, or if the activity outer membrane receptor protein precursor is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 87 to 189-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a adenylosuccinate lyase, or if the activity of the polypeptide B1131, preferably represented by SEQ ID NO. 144612, or a homolog or fragment thereof, or if the activity of a polypeptide encoded by a nucleic acid molecule comprising the nucleic acid SEQ ID NO. 144611, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 144611 or polypeptide SEQ ID NO. 144612, respectively, or a homolog or a fragment thereof, is increased or generated, or if the activity adenylosuccinate lyase is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 18 to 23-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a isocitrate dehydrogenase, or if the activity of the polypeptide B1136, preferably represented by SEQ ID NO. 36972, or a homolog or fragment thereof, or if the activity of a polypeptide encoded by a nucleic acid molecule comprising the nucleic acid SEQ ID NO. 36971, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 36971 or polypeptide SEQ ID NO. 36972, respectively, or a homolog or a fragment thereof, is increased or generated, or if the activity isocitrate dehydrogenase is increased or generated in a non-human organism, like a microorganism or a plant cell, plant or part thereof, especially with plastidic localization, whereby the respective line disclose in column 7 the fine chemical glucose. For example, an increase of the glucose of at least 1 percent, particularly in a range of 50 to 104-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a b1137-protein, or if the activity of the polypeptide B1137, preferably represented by SEQ ID NO. 37391, or a homolog or fragment thereof, or if the activity of a polypeptide encoded by a nucleic acid molecule comprising the nucleic acid SEQ ID NO. 37390, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 37390 or polypeptide SEQ ID NO. 37391, respectively, or a homolog or a fragment thereof, is increased or generated, or if the activity b1137-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 20 to 65-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a sodium/proton antiporter, or if the activity of the polypeptide B1186, preferably represented by SEQ ID NO. 37401, or a homolog or fragment thereof, or if the activity of a polypeptide encoded by a nucleic acid molecule comprising the nucleic acid SEQ ID NO. 37400, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 37400 or polypeptide SEQ ID NO. 37401, respectively, or a homolog or a fragment thereof, is increased or generated, or if the activity sodium/proton antiporter is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 24 to 126-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a b1214-protein, or if the activity of the polypeptide B1214, preferably represented by SEQ ID NO. 144858, or a homolog or fragment thereof, or if the activity of a polypeptide encoded by a nucleic acid molecule comprising the nucleic acid SEQ ID NO. 144857, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 144857 or polypeptide SEQ ID NO. 144858, respectively, or a homolog or a fragment thereof, is increased or generated, or if the activity b1214-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 43 to 71-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a B1214-protein, or if the activity of the polypeptide B1214, preferably represented by SEQ ID NO. 144858, or a homolog or fragment thereof, or if the activity of a polypeptide encoded by a nucleic acid molecule comprising the nucleic acid SEQ ID NO. 144857, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 144857 or polypeptide SEQ ID NO. 144858, respectively, or a homolog or a fragment thereof, is increased or generated, or if the activity b1214-protein is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 40 to 58-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a b1219-protein, or if the activity of the polypeptide B1219, preferably represented by SEQ ID NO. 112073, or a homolog or fragment thereof, or if the activity of a polypeptide encoded by a nucleic acid molecule comprising the nucleic acid SEQ ID NO. 112072, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 112072 or polypeptide SEQ ID NO. 112073, respectively, or a homolog or a fragment thereof, is increased or generated, or if the activity b1219-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 40 to 58-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a b1219-protein, or if the activity of the polypeptide B1219, preferably represented by SEQ ID NO. 112073, or a homolog or fragment thereof, or if the activity of a polypeptide encoded by a nucleic acid molecule comprising the nucleic acid SEQ ID NO. 112072, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 112072 or polypeptide SEQ ID NO. 112073, respectively, or a homolog or a fragment thereof, is increased or generated, or if the activity b1219-protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 36 to 38-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a b1219-protein, or if the activity of the polypeptide B1219, preferably represented by SEQ ID NO. 112073, or a homolog or fragment thereof, or if the activity of a polypeptide encoded by a nucleic acid molecule comprising the nucleic acid SEQ ID NO. 112072, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 112072 or polypeptide SEQ ID NO. 112073, respectively, or a homolog or a fragment thereof, is increased or generated, or if the activity b1219-protein is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 35 to 76-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a b1234-protein, or if the activity of the polypeptide B1234, preferably represented by SEQ ID NO. 112112, or a homolog or fragment thereof, or if the activity of a polypeptide encoded by a nucleic acid molecule comprising the nucleic acid SEQ ID NO. 112111, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 112111 or polypeptide SEQ ID NO. 112112, respectively, or a homolog or a fragment thereof, is increased or generated, or if the activity b1234-protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 26 to 38-percent is conferred as compared to a corresponding non-transformed wild type non-human organism.

Accordingly, in one embodiment, an increase of galactinol in a non-human organism, as compared to a corresponding non-transformed wild type non-human organism, is conferred in the process of the invention, if the activity of a polypeptide showing the activity of a cardiolipin synthase, or if the activity of the polypeptide B1249, preferably represented by SEQ ID NO. 98779, or a homolog or fragment thereof, or if the activity of a polypeptide encoded by a nucleic acid molecule comprising the nucleic acid SEQ ID NO. 98778, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 98778 or polypeptide SEQ ID NO. 98779, respectively, or a homolog or a fragment thereof, is increased or generated, or if the activity cardiolipin synthase is increased or generated in a non-human organism, like a microorganism or a plant cell, plant or part thereof, especially with plastidic localization, whereby the respective line disclose in column 7 the fine chemical galactinol. For example, an increase of the galactinol of at least 1 percent, particularly in a range of 40 to 153-percent is conferred as compared to a corresponding non-transformed wild type non-human organism.

Accordingly, in one embodiment, an increase of galactitol in a non-human organism, as compared to a corresponding non-transformed wild type non-human organism, is conferred in the process of the invention, if the activity of a polypeptide showing the activity of a cardiolipin synthase, or if the activity of the polypeptide B1249, preferably represented by SEQ ID NO. 98779, or a homolog or fragment thereof, or if the activity of a polypeptide encoded by a nucleic acid molecule comprising the nucleic acid SEQ ID NO. 98778, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 98778 or polypeptide SEQ ID NO. 98779, respectively, or a homolog or a fragment thereof, is increased or generated, or if the activity cardiolipin synthase is increased or generated in a non-human organism, like a microorganism or a plant cell, plant or part thereof, especially with plastidic localization, whereby the respective line disclose in column 7 the fine chemical galactitol. For example, an increase of the galactitol of at least 1 percent, particularly in a range of 65 to 272-percent is conferred as compared to a corresponding non-transformed wild type non-human organism.

Accordingly, in one embodiment, an increase of sorbitol in a non-human organism, as compared to a corresponding non-transformed wild type non-human organism, is conferred in the process of the invention, if the activity of a polypeptide showing the activity of a cardiolipin synthase, or if the activity of the polypeptide B1249, preferably represented by SEQ ID NO. 98779, or a homolog or fragment thereof, or if the activity of a polypeptide encoded by a nucleic acid molecule comprising the nucleic acid SEQ ID NO. 98778, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 98778 or polypeptide SEQ ID NO. 98779, respectively, or a homolog or a fragment thereof, is increased or generated, or if the activity cardiolipin synthase is increased or generated in a non-human organism, like a microorganism or a plant cell, plant or part thereof, especially with plastidic localization, whereby the respective line disclose in column 7 the fine chemical sorbitol. For example, an increase of the sorbitol of at least 1 percent, particularly in a range of 65 to 272-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a intracellular septation protein, or if the activity of the polypeptide B1254, preferably represented by SEQ ID NO. 144895, or a homolog or fragment thereof, or if the activity of a polypeptide encoded by a nucleic acid molecule comprising the nucleic acid SEQ ID NO. 144894, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 144894 or polypeptide SEQ ID NO. 144895, respectively, or a homolog or a fragment thereof, is increased or generated, or if the activity intracellular septation protein is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 37 to 56-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a membrane protein, or if the activity of the polypeptide B1255, preferably represented by SEQ ID NO. 37484, or a homolog or fragment thereof, or if the activity of a polypeptide encoded by a nucleic acid molecule comprising the nucleic acid SEQ ID NO. 37483, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 37483 or polypeptide SEQ ID NO. 37484, respectively, or a homolog or a fragment thereof, is increased or generated, or if the activity membrane protein is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 80 to 727-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a membrane protein, or if the activity of the polypeptide B1255, preferably represented by SEQ ID NO. 37484, or a homolog or fragment thereof, or if the activity of a polypeptide encoded by a nucleic acid molecule comprising the nucleic acid SEQ ID NO. 37483, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 37483 or polypeptide SEQ ID NO. 37484, respectively, or a homolog or a fragment thereof, is increased or generated, or if the activity membrane protein is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 66 to 597-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a membrane protein, or if the activity of the polypeptide B1255, preferably represented by SEQ ID NO. 37484, or a homolog or fragment thereof, or if the activity of a polypeptide encoded by a nucleic acid molecule comprising the nucleic acid SEQ ID NO. 37483, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 37483 or polypeptide SEQ ID NO. 37484, respectively, or a homolog or a fragment thereof, is increased or generated, or if the activity membrane protein is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 82 to 369-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a membrane protein, or if the activity of the polypeptide B1255, preferably represented by SEQ ID NO. 37484, or a homolog or fragment thereof, or if the activity of a polypeptide encoded by a nucleic acid molecule comprising the nucleic acid SEQ ID NO. 37483, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 37483 or polypeptide SEQ ID NO. 37484, respectively, or a homolog or a fragment thereof, is increased or generated, or if the activity membrane protein is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 33 to 52-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a tryptophan biosynthesis protein, or if the activity of the polypeptide B1262, preferably represented by SEQ ID NO. 76158, or a homolog or fragment thereof, or if the activity of a polypeptide encoded by a nucleic acid molecule comprising the nucleic acid SEQ ID NO. 76157, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 76157 or polypeptide SEQ ID NO. 76158, respectively, or a homolog or a fragment thereof, is increased or generated, or if the activity tryptophan biosynthesis protein is increased or generated in a non-human organism, like a microorganism or a plant cell, plant or part thereof, especially with plastidic localization, whereby the respective line disclose in column 7 the fine chemical myo-inositol. For example, an increase of the myo-inositol of at least 1 percent, particularly in a range of 32 to 577-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a b1285-protein, or if the activity of the polypeptide B1285, preferably represented by SEQ ID NO. 134656, or a homolog or fragment thereof, or if the activity of a polypeptide encoded by a nucleic acid molecule comprising the nucleic acid SEQ ID NO. 134655, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 134655 or polypeptide SEQ ID NO. 134656, respectively, or a homolog or a fragment thereof, is increased or generated, or if the activity b1285-protein is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 71 to 127-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a phage-related repressor protein, or if the activity of the polypeptide B1356, preferably represented by SEQ ID NO. 124558, or a homolog or fragment thereof, or if the activity of a polypeptide encoded by a nucleic acid molecule comprising the nucleic acid SEQ ID NO. 124557, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 124557 or polypeptide SEQ ID NO. 124558, respectively, or a homolog or a fragment thereof, is increased or generated, or if the activity phage-related repressor protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1-percent, particularly in a range of 19 to 20-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a b1357-protein, or if the activity of the polypeptide B1357, preferably represented by SEQ ID NO. 145067, or a homolog or fragment thereof, or if the activity of a polypeptide encoded by a nucleic acid molecule comprising the nucleic acid SEQ ID NO. 145066, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 145066 or polypeptide SEQ ID NO. 145067, respectively, or a homolog or a fragment thereof, is increased or generated, or if the activity b1357-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 20 to 44-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a phenylacetic acid degradation protein, or if the activity of the polypeptide B1390, preferably represented by SEQ ID NO. 134698, or a homolog or fragment thereof, or if the activity of a polypeptide encoded by a nucleic acid molecule comprising the nucleic acid SEQ ID NO. 134697, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 134697 or polypeptide SEQ ID NO. 134698, respectively, or a homolog or a fragment thereof, is increased or generated, or if the activity phenylacetic acid degradation protein is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 57 to 120-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a phenylacetic acid degradation protein, or if the activity of the polypeptide B1391, preferably represented by SEQ ID NO. 131490, or a homolog or fragment thereof, or if the activity of a polypeptide encoded by a nucleic acid molecule comprising the nucleic acid SEQ ID NO. 131489, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 131489 or polypeptide SEQ ID NO. 131490, respectively, or a homolog or a fragment thereof, is increased or generated, or if the activity phenylacetic acid degradation protein is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 43 to 95-percent is conferred as compared to a corresponding non-transformed wild type non-human organism.

Accordingly, in one embodiment, an increase of sedoheptulose-7-phosphate in a non-human organism, as compared to a corresponding non-transformed wild type non-human organism, is conferred in the process of the invention, if the activity of a polypeptide showing the activity of a b1410-protein, or if the activity of the polypeptide B1410, preferably represented by SEQ ID NO. 145092, or a homolog or fragment thereof, or if the activity of a polypeptide encoded by a nucleic acid molecule comprising the nucleic acid SEQ ID NO. 145091, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 145091 or polypeptide SEQ ID NO. 145092, respectively, or a homolog or a fragment thereof, is increased or generated, or if the activity b1410-protein is increased or generated in a non-human organism, like a microorganism or a plant cell, plant or part thereof, especially with plastidic localization, whereby the respective line disclose in column 7 the fine chemical sedoheptulose-7-phosphate. For example, an increase of the sedoheptulose-7-phosphate of at least 1 percent, particularly in a range of 43 to 112-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a b1425-protein, or if the activity of the polypeptide B1425, preferably represented by SEQ ID NO. 134757, or a homolog or fragment thereof, or if the activity of a polypeptide encoded by a nucleic acid molecule comprising the nucleic acid SEQ ID NO. 134756, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 134756 or polypeptide SEQ ID NO. 134757, respectively, or a homolog or a fragment thereof, is increased or generated, or if the activity b1425-protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 25 to 36-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a lipoprotein precursor, or if the activity of the polypeptide B1431, preferably represented by SEQ ID NO. 38267, or a homolog or fragment thereof, or if the activity of a polypeptide encoded by a nucleic acid molecule comprising the nucleic acid SEQ ID NO. 38266, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 38266 or polypeptide SEQ ID NO. 38267, respectively, or a homolog or a fragment thereof, is increased or generated, or if the activity lipoprotein precursor is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 31 to 52-percent is conferred as compared to a corresponding non-transformed wild type non-human organism.

Accordingly, in one embodiment, an increase of sedoheptulose-7-phosphate in a non-human organism, as compared to a corresponding non-transformed wild type non-human organism, is conferred in the process of the invention, if the activity of a polypeptide showing the activity of a lipoprotein precursor, or if the activity of the polypeptide B1431, preferably represented by SEQ ID NO. 38267, or a homolog or fragment thereof, or if the activity of a polypeptide encoded by a nucleic acid molecule comprising the nucleic acid SEQ ID NO. 38266, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 38266 or polypeptide SEQ ID NO. 38267, respectively, or a homolog or a fragment thereof, is increased or generated, or if the activity lipoprotein precursor is increased or generated in a non-human organism, like a microorganism or a plant cell, plant or part thereof, especially with plastidic localization, whereby the respective line disclose in column 7 the fine chemical sedoheptulose-7-phosphate. For example, an increase of the sedoheptulose-7-phosphate of at least 1 percent, particularly in a range of 32 to 52-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a lipoprotein precursor, or if the activity of the polypeptide B1431, preferably represented by SEQ ID NO. 38267, or a homolog or fragment thereof, or if the activity of a polypeptide encoded by a nucleic acid molecule comprising the nucleic acid SEQ ID NO. 38266, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 38266 or polypeptide SEQ ID NO. 38267, respectively, or a homolog or a fragment thereof, is increased or generated, or if the activity lipoprotein precursor is increased or generated in a non-human organism, like a microorganism or a plant cell, plant or part thereof, especially with plastidic localization, whereby the respective line disclose in column 7 the fine chemical 3,4,5-trihydroxypentanoic acid. For example, an increase of the 3,4,5-trihydroxypentanoic acid of at least 1 percent, particularly in a range of 37 to 89-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a b1445-protein, or if the activity of the polypeptide B1445, preferably represented by SEQ ID NO. 38290, or a homolog or fragment thereof, or if the activity of a polypeptide encoded by a nucleic acid molecule comprising the nucleic acid SEQ ID NO. 38289, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 38289 or polypeptide SEQ ID NO. 38290, respectively, or a homolog or a fragment thereof, is increased or generated, or if the activity b1445-protein is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 47 to 50-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a b1470-protein, or if the activity of the polypeptide B1470, preferably represented by SEQ ID NO. 115057, or a homolog or fragment thereof, or if the activity of a polypeptide encoded by a nucleic acid molecule comprising the nucleic acid SEQ ID NO. 115056, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 115056 or polypeptide SEQ ID NO. 115057, respectively, or a homolog or a fragment thereof, is increased or generated, or if the activity b1470-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 18 to 44-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a b1522-protein, or if the activity of the polypeptide B1522, preferably represented by SEQ ID NO. 7948, or a homolog or fragment thereof, or if the activity of a polypeptide encoded by a nucleic acid molecule comprising the nucleic acid SEQ ID NO. 7947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 7947 or polypeptide SEQ ID NO. 7948, respectively, or a homolog or a fragment thereof, is increased or generated, or if the activity b1522-protein is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 83 to 364-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a b1522-protein, or if the activity of the polypeptide B1522, preferably represented by SEQ ID NO. 7948, or a homolog or fragment thereof, or if the activity of a polypeptide encoded by a nucleic acid molecule comprising the nucleic acid SEQ ID NO. 7947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 7947 or polypeptide SEQ ID NO. 7948, respectively, or a homolog or a fragment thereof, is increased or generated, or if the activity b1522-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 20 to 41-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a b1556-protein, or if the activity of the polypeptide B1556, preferably represented by SEQ ID NO. 145143, or a homolog or fragment thereof, or if the activity of a polypeptide encoded by a nucleic acid molecule comprising the nucleic acid SEQ ID NO. 145142, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 145142 or polypeptide SEQ ID NO. 145143, respectively, or a homolog or a fragment thereof, is increased or generated, or if the activity b1556-protein is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 35 to 57-percent is conferred as compared to a corresponding non-transformed wild type non-human organism.

Accordingly, in one embodiment, an increase of sedoheptulose-7-phosphate in a non-human organism, as compared to a corresponding non-transformed wild type non-human organism, is conferred in the process of the invention, if the activity of a polypeptide showing the activity of a b1556-protein, or if the activity of the polypeptide B1556, preferably represented by SEQ ID NO. 145143, or a homolog or fragment thereof, or if the activity of a polypeptide encoded by a nucleic acid molecule comprising the nucleic acid SEQ ID NO. 145142, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 145142 or polypeptide SEQ ID NO. 145143, respectively, or a homolog or a fragment thereof, is increased or generated, or if the activity b1556-protein is increased or generated in a non-human organism, like a microorganism or a plant cell, plant or part thereof, especially with plastidic localization, whereby the respective line disclose in column 7 the fine chemical sedoheptulose-7-phosphate. For example, an increase of the sedoheptulose-7-phosphate of at least 1 percent, particularly in a range of 86 to 382-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a b1583-protein, or if the activity of the polypeptide B1583, preferably represented by SEQ ID NO. 78139, or a homolog or fragment thereof, or if the activity of a polypeptide encoded by a nucleic acid molecule comprising the nucleic acid SEQ ID NO. 78138, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 78138 or polypeptide SEQ ID NO. 78139, respectively, or a homolog or a fragment thereof, is increased or generated, or if the activity b1583-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 23 to 46-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a FeS subunit of oxidoreductase, or if the activity of the polypeptide B1589, preferably represented by SEQ ID NO. 78155, or a homolog or fragment thereof, or if the activity of a polypeptide encoded by a nucleic acid molecule comprising the nucleic acid SEQ ID NO. 78154, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 78154 or polypeptide SEQ ID NO. 78155, respectively, or a homolog or a fragment thereof, is increased or generated, or if the activity Fe—S subunit of oxidoreductase is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 50 to 114-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a Fe—S subunit of oxidoreductase, or if the activity of the polypeptide B1589, preferably represented by SEQ ID NO. 78155, or a homolog or fragment thereof, or if the activity of a polypeptide encoded by a nucleic acid molecule comprising the nucleic acid SEQ ID NO. 78154, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 78154 or polypeptide SEQ ID NO. 78155, respectively, or a homolog or a fragment thereof, is increased or generated, or if the activity Fe—S subunit of oxidoreductase is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 33 to 122-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a acid shock protein, or if the activity of the polypeptide B1597, preferably represented by SEQ ID NO. 38301, or a homolog or fragment thereof, or if the activity of a polypeptide encoded by a nucleic acid molecule comprising the nucleic acid SEQ ID NO. 38300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 38300 or polypeptide SEQ ID NO. 38301, respectively, or a homolog or a fragment thereof, is increased or generated, or if the activity acid shock protein is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 78 to 250-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a acid shock protein, or if the activity of the polypeptide B1597, preferably represented by SEQ ID NO. 38301, or a homolog or fragment thereof, or if the activity of a polypeptide encoded by a nucleic acid molecule comprising the nucleic acid SEQ ID NO. 38300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 38300 or polypeptide SEQ ID NO. 38301, respectively, or a homolog or a fragment thereof, is increased or generated, or if the activity acid shock protein is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 86 to 187-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a acid shock protein, or if the activity of the polypeptide B1597, preferably represented by SEQ ID NO. 38301, or a homolog or fragment thereof, or if the activity of a polypeptide encoded by a nucleic acid molecule comprising the nucleic acid SEQ ID NO. 38300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 38300 or polypeptide SEQ ID NO. 38301, respectively, or a homolog or a fragment thereof, is increased or generated, or if the activity acid shock protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 47 to 249-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a acid shock protein, or if the activity of the polypeptide B1597, preferably represented by SEQ ID NO. 38301, or a homolog or fragment thereof, or if the activity of a polypeptide encoded by a nucleic acid molecule comprising the nucleic acid SEQ ID NO. 38300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 38300 or polypeptide SEQ ID NO. 38301, respectively, or a homolog or a fragment thereof, is increased or generated, or if the activity acid shock protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 33 to 81-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a acid shock protein, or if the activity of the polypeptide B1597, preferably represented by SEQ ID NO. 38301, or a homolog or fragment thereof, or if the activity of a polypeptide encoded by a nucleic acid molecule comprising the nucleic acid SEQ ID NO. 38300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 38300 or polypeptide SEQ ID NO. 38301, respectively, or a homolog or a fragment thereof, is increased or generated, or if the activity acid shock protein is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 65 to 199-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a phosphotransferase system component, or if the activity of the polypeptide B1621, preferably represented by SEQ ID NO. 124572, or a homolog or fragment thereof, or if the activity of a polypeptide encoded by a nucleic acid molecule comprising the nucleic acid SEQ ID NO. 124571, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 124571 or polypeptide SEQ ID NO. 124572, respectively, or a homolog or a fragment thereof, is increased or generated, or if the activity phosphotransferase system component is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 107 to 424-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a phosphotransferase system component, or if the activity of the polypeptide B1621, preferably represented by SEQ ID NO. 124572, or a homolog or fragment thereof, or if the activity of a polypeptide encoded by a nucleic acid molecule comprising the nucleic acid SEQ ID NO. 124571, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 124571 or polypeptide SEQ ID NO. 124572, respectively, or a homolog or a fragment thereof, is increased or generated, or if the activity phosphotransferase system component is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 77 to 119-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a electron transport complex protein, or if the activity of the polypeptide B1627, preferably represented by SEQ ID NO. 38346, or a homolog or fragment thereof, or if the activity of a polypeptide encoded by a nucleic acid molecule comprising the nucleic acid SEQ ID NO. 38345, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 38345 or polypeptide SEQ ID NO. 38346, respectively, or a homolog or a fragment thereof, is increased or generated, or if the activity electron transport complex protein is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 72 to 186-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a electron transport complex protein, or if the activity of the polypeptide B1627, preferably represented by SEQ ID NO. 38346, or a homolog or fragment thereof, or if the activity of a polypeptide encoded by a nucleic acid molecule comprising the nucleic acid SEQ ID NO. 38345, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 38345 or polypeptide SEQ ID NO. 38346, respectively, or a homolog or a fragment thereof, is increased or generated, or if the activity electron transport complex protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1-percent, particularly in a range of 32 to 78-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a electron transport complex protein, or if the activity of the polypeptide B1627, preferably represented by SEQ ID NO. 38346, or a homolog or fragment thereof, or if the activity of a polypeptide encoded by a nucleic acid molecule comprising the nucleic acid SEQ ID NO. 38345, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 38345 or polypeptide SEQ ID NO. 38346, respectively, or a homolog or a fragment thereof, is increased or generated, or if the activity electron transport complex protein is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 55 to 78-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a methylglyoxalase, or if the activity of the polypeptide B1651, preferably represented by SEQ ID NO. 78266, or a homolog or fragment thereof, or if the activity of a polypeptide encoded by a nucleic acid molecule comprising the nucleic acid SEQ ID NO. 78265, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 78265 or polypeptide SEQ ID NO. 78266, respectively, or a homolog or a fragment thereof, is increased or generated, or if the activity methylglyoxalase is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 25 to 41-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a b1670-protein, or if the activity of the polypeptide B1670, preferably represented by SEQ ID NO. 78754, or a homolog or fragment thereof, or if the activity of a polypeptide encoded by a nucleic acid molecule comprising the nucleic acid SEQ ID NO. 78753, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 78753 or polypeptide SEQ ID NO. 78754, respectively, or a homolog or a fragment thereof, is increased or generated, or if the activity b1670-protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 30 to 45-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a ABC transporter permease protein, or if the activity of the polypeptide B1755, preferably represented by SEQ ID NO. 78853, or a homolog or fragment thereof, or if the activity of a polypeptide encoded by a nucleic acid molecule comprising the nucleic acid SEQ ID NO. 78852, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 78852 or polypeptide SEQ ID NO. 78853, respectively, or a homolog or a fragment thereof, is increased or generated, or if the activity ABC transporter permease protein is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 83 to 94-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a transport protein, or if the activity of the polypeptide B1791, preferably represented by SEQ ID NO. 78884, or a homolog or fragment thereof, or if the activity of a polypeptide encoded by a nucleic acid molecule comprising the nucleic acid SEQ ID NO. 78883, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 78883 or polypeptide SEQ ID NO. 78884, respectively, or a homolog or a fragment thereof, is increased or generated, or if the activity transport protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 25 to 344-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a b1796-protein, or if the activity of the polypeptide B1796, preferably represented by SEQ ID NO. 99197, or a homolog or fragment thereof, or if the activity of a polypeptide encoded by a nucleic acid molecule comprising the nucleic acid SEQ ID NO. 99196, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 99196 or polypeptide SEQ ID NO. 99197, respectively, or a homolog or a fragment thereof, is increased or generated, or if the activity b1796-protein is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 85 to 93-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a paraaminobenzoate synthase componentl, or if the activity of the polypeptide B1812, preferably represented by SEQ ID NO. 145181, or a homolog or fragment thereof, or if the activity of a polypeptide encoded by a nucleic acid molecule comprising the nucleic acid SEQ ID NO. 145180, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 145180 or polypeptide SEQ ID NO. 145181, respectively, or a homolog or a fragment thereof, is increased or generated, or if the activity para-aminobenzoate synthase componentl is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 33 to 49-percent is conferred as compared to a corresponding non-transformed wild type non-human organism.

Accordingly, in one embodiment, an increase of galactitol in a non-human organism, as compared to a corresponding non-transformed wild type non-human organism, is conferred in the process of the invention, if the activity of a polypeptide showing the activity of a L-serine dehydratase, or if the activity of the polypeptide B1814, preferably represented by SEQ ID NO. 8034, or a homolog or fragment thereof, or if the activity of a polypeptide encoded by a nucleic acid molecule comprising the nucleic acid SEQ ID NO. 8033, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 8033 or polypeptide SEQ ID NO. 8034, respectively, or a homolog or a fragment thereof, is increased or generated, or if the activity L-serine dehydratase is increased or generated in a non-human organism, like a microorganism or a plant cell, plant or part thereof, especially with plastidic localization, whereby the respective line disclose in column 7 the fine chemical galactitol. For example, an increase of the galactitol of at least 1 percent, particularly in a range of 55 to 2661-percent is conferred as compared to a corresponding non-transformed wild type non-human organism.

Accordingly, in one embodiment, an increase of sorbitol in a non-human organism, as compared to a corresponding non-transformed wild type non-human organism, is conferred in the process of the invention, if the activity of a polypeptide showing the activity of a L-serine dehydratase, or if the activity of the polypeptide B1814, preferably represented by SEQ ID NO. 8034, or a homolog or fragment thereof, or if the activity of a polypeptide encoded by a nucleic acid molecule comprising the nucleic acid SEQ ID NO. 8033, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 8033 or polypeptide SEQ ID NO. 8034, respectively, or a homolog or a fragment thereof, is increased or generated, or if the activity L-serine dehydratase is increased or generated in a non-human organism, like a microorganism or a plant cell, plant or part thereof, especially with plastidic localization, whereby the respective line disclose in column 7 the fine chemical sorbitol. For example, an increase of the sorbitol of at least 1 percent, particularly in a range of 55 to 2661-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a protease, or if the activity of the polypeptide B1845, preferably represented by SEQ ID NO. 38574, or a homolog or fragment thereof, or if the activity of a polypeptide encoded by a nucleic acid molecule comprising the nucleic acid SEQ ID NO. 38573, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 38573 or polypeptide SEQ ID NO. 38574, respectively, or a homolog or a fragment thereof, is increased or generated, or if the activity protease is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 26 to 51-percent is conferred as compared to a corresponding non-transformed wild type non-human organism.

Accordingly, in one embodiment, an increase of erythrol in a non-human organism, as compared to a corresponding non-transformed wild type non-human organism, is conferred in the process of the invention, if the activity of a polypeptide showing the activity of a pyruvate kinase, or if the activity of the polypeptide B1854, preferably represented by SEQ ID NO. 8364, or a homolog or fragment thereof, or if the activity of a polypeptide encoded by a nucleic acid molecule comprising the nucleic acid SEQ ID NO. 8363, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 8363 or polypeptide SEQ ID NO. 8364, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line disclose in column 7 the fine chemical erythrol. For example, an increase of the erythrol of at least 1 percent, particularly in a range of 140 to 330-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a pyruvate kinase, or if the activity of the polypeptide B1854, preferably represented by SEQ ID NO. 8364, or a homolog or fragment thereof, or if the activity of a polypeptide encoded by a nucleic acid molecule comprising the nucleic acid SEQ ID NO. 8363, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 8363 or polypeptide SEQ ID NO. 8364, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line disclose in column 7 the fine chemical myo-inositol. For example, an increase of the myo-inositol of at least 1 percent, particularly in a range of 44 to 81-percent is conferred as compared to a corresponding non-transformed wild type non-human organism.

Accordingly, in one embodiment, an increase of sorbitol in a non-human organism, as compared to a corresponding non-transformed wild type non-human organism, is conferred in the process of the invention, if the activity of a polypeptide showing the activity of a pyruvate kinase, or if the activity of the polypeptide B1854, preferably represented by SEQ ID NO. 8364, or a homolog or fragment thereof, or if the activity of a polypeptide encoded by a nucleic acid molecule comprising the nucleic acid SEQ ID NO. 8363, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 8363 or polypeptide SEQ ID NO. 8364, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line disclose in column 7 the fine chemical sorbitol. For example, an increase of the sorbitol of at least 1 percent, particularly in a range of 200 to 414-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a dATP pyrophosphohydrolase, or if the activity of the polypeptide B1865, preferably represented by SEQ ID NO. 134784, or a homolog or fragment thereof, or if the activity of a polypeptide encoded by a nucleic acid molecule comprising the nucleic acid SEQ ID NO. 134783, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 134783 or polypeptide SEQ ID NO. 134784, respectively, or a homolog or a fragment thereof, is increased or generated, or if the activity dATP pyrophosphohydrolase is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 32 to 105-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a trehalose-6-phosphate synthase, or if the activity of the polypeptide B1896, preferably represented by SEQ ID NO. 145412, or a homolog or fragment thereof, or if the activity of a polypeptide encoded by a nucleic acid molecule comprising the nucleic acid SEQ ID NO. 145411, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 145411 or polypeptide SEQ ID NO. 145412, respectively, or a homolog or a fragment thereof, is increased or generated, or if the activity trehalose-6-phosphate synthase is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1-percent, particularly in a range of 19 to 74-percent is conferred as compared to a corresponding non-transformed wild type non-human organism.

Accordingly, in one embodiment, an increase of trehalose in a non-human organism, as compared to a corresponding non-transformed wild type non-human organism, is conferred in the process of the invention, if the activity of a polypeptide showing the activity of a trehalose-6-phosphate synthase, or if the activity of the polypeptide B1896, preferably represented by SEQ ID NO. 145412, or a homolog or fragment thereof, or if the activity of a polypeptide encoded by a nucleic acid molecule comprising the nucleic acid SEQ ID NO. 145411, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 145411 or polypeptide SEQ ID NO. 145412, respectively, or a homolog or a fragment thereof, is increased or generated, or if the activity trehalose-6-phosphate synthase is increased or generated in a non-human organism, like a microorganism or a plant cell, plant or part thereof, especially with non-targeted localization, whereby the respective line disclose in column 7 the fine chemical trehalose. For example, an increase of the trehalose of at least 1 percent, particularly in a range of 294 to 441-percent is conferred as compared to a corresponding non-transformed wild type non-human organism.

Accordingly, in one embodiment, an increase of erythrol in a non-human organism, as compared to a corresponding non-transformed wild type non-human organism, is conferred in the process of the invention, if the activity of a polypeptide showing the activity of a trehalosephosphatase, or if the activity of the polypeptide B1897, preferably represented by SEQ ID NO. 79057, or a homolog or fragment thereof, or if the activity of a polypeptide encoded by a nucleic acid molecule comprising the nucleic acid SEQ ID NO. 79056, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 79056 or polypeptide SEQ ID NO. 79057, respectively, or a homolog or a fragment thereof, is increased or generated, or if the activity trehalose-phosphatase is increased or generated in a non-human organism, like a microorganism or a plant cell, plant or part thereof, especially with plastidic localization, whereby the respective line disclose in column 7 the fine chemical erythrol. For example, an increase of the erythrol of at least 1 percent, particularly in a range of 139 to 3201-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a trehalosephosphatase, or if the activity of the polypeptide B1897, preferably represented by SEQ ID NO. 79057, or a homolog or fragment thereof, or if the activity of a polypeptide encoded by a nucleic acid molecule comprising the nucleic acid SEQ ID NO. 79056, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 79056 or polypeptide SEQ ID NO. 79057, respectively, or a homolog or a fragment thereof, is increased or generated, or if the activity trehalose-phosphatase is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 202 to 2339-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a trehalosephosphatase, or if the activity of the polypeptide B1897, preferably represented by SEQ ID NO. 79057, or a homolog or fragment thereof, or if the activity of a polypeptide encoded by a nucleic acid molecule comprising the nucleic acid SEQ ID NO. 79056, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 79056 or polypeptide SEQ ID NO. 79057, respectively, or a homolog or a fragment thereof, is increased or generated, or if the activity trehalose-phosphatase is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 120 to 1091-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a trehalosephosphatase, or if the activity of the polypeptide B1897, preferably represented by SEQ ID NO. 79057, or a homolog or fragment thereof, or if the activity of a polypeptide encoded by a nucleic acid molecule comprising the nucleic acid SEQ ID NO. 79056, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 79056 or polypeptide SEQ ID NO. 79057, respectively, or a homolog or a fragment thereof, is increased or generated, or if the activity trehalose-phosphatase is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 38 to 103-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a b1898-protein, or if the activity of the polypeptide B1898, preferably represented by SEQ ID NO. 38768, or a homolog or fragment thereof, or if the activity of a polypeptide encoded by a nucleic acid molecule comprising the nucleic acid SEQ ID NO. 38767, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 38767 or polypeptide SEQ ID NO. 38768, respectively, or a homolog or a fragment thereof, is increased or generated, or if the activity b1898-protein is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 55 to 329-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a acetyltransferase, or if the activity of the polypeptide B1932, preferably represented by SEQ ID NO. 131800, or a homolog or fragment thereof, or if the activity of a polypeptide encoded by a nucleic acid molecule comprising the nucleic acid SEQ ID NO. 131799, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 131799 or polypeptide SEQ ID NO. 131800, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 19 to 107-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a acetyltransferase, or if the activity of the polypeptide B1932, preferably represented by SEQ ID NO. 131800, or a homolog or fragment thereof, or if the activity of a polypeptide encoded by a nucleic acid molecule comprising the nucleic acid SEQ ID NO. 131799, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 131799 or polypeptide SEQ ID NO. 131800, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 33 to 84-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a flagellar M-ring protein, or if the activity of the polypeptide B1938, preferably represented by SEQ ID NO. 134865, or a homolog or fragment thereof, or if the activity of a polypeptide encoded by a nucleic acid molecule comprising the nucleic acid SEQ ID NO. 134864, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 134864 or polypeptide SEQ ID NO. 134865, respectively, or a homolog or a fragment thereof, is increased or generated, or if the activity flagellar M-ring protein is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 65 to 201-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a flagellar M-ring protein, or if the activity of the polypeptide B1938, preferably represented by SEQ ID NO. 134865, or a homolog or fragment thereof, or if the activity of a polypeptide encoded by a nucleic acid molecule comprising the nucleic acid SEQ ID NO. 134864, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 134864 or polypeptide SEQ ID NO. 134865, respectively, or a homolog or a fragment thereof, is increased or generated, or if the activity flagellar M-ring protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 28 to 190-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a O-antigen chain length determinant, or if the activity of the polypeptide B2027, preferably represented by SEQ ID NO. 79218, or a homolog or fragment thereof, or if the activity of a polypeptide encoded by a nucleic acid molecule comprising the nucleic acid SEQ ID NO. 79217, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 79217 or polypeptide SEQ ID NO. 79218, respectively, or a homolog or a fragment thereof, is increased or generated, or if the activity O-antigen chain length determinant is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 156 to 232-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a b2032-protein, or if the activity of the polypeptide B2032, preferably represented by SEQ ID NO. 8921, or a homolog or fragment thereof, or if the activity of a polypeptide encoded by a nucleic acid molecule comprising the nucleic acid SEQ ID NO. 8920, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 8920 or polypeptide SEQ ID NO. 8921, respectively, or a hornolog or a fragment thereof, is increased or generated, or if the activity b2032-protein is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 61 to 157-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a b2032-protein, or if the activity of the polypeptide B2032, preferably represented by SEQ ID NO. 8921, or a homolog or fragment thereof, or if the activity of a polypeptide encoded by a nucleic acid molecule comprising the nucleic acid SEQ ID NO. 8920, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 8920 or polypeptide SEQ ID NO. 8921, respectively, or a homolog or a fragment thereof, is increased or generated, or if the activity b2032-protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 27 to 212-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a colanic acid biosynthesis protein, or if the activity of the polypeptide B2043, preferably represented by SEQ ID NO. 112194, or a homolog or fragment thereof, or if the activity of a polypeptide encoded by a nucleic acid molecule comprising the nucleic acid SEQ ID NO. 112193, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 112193 or polypeptide SEQ ID NO. 112194, respectively, or a homolog or a fragment thereof, is increased or generated, or if the activity colanic acid biosynthesis protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1-percent, particularly in a range of 18 to 35-percent is conferred as compared to a corresponding non-transformed wild type non-human organism.

Accordingly, in one embodiment, an increase of fucose in a non-human organism, as compared to a corresponding non-transformed wild type non-human organism, is conferred in the process of the invention, if the activity of a polypeptide showing the activity of a GDP-mannose 4,6-dehydratase, or if the activity of the polypeptide B2053, preferably represented by SEQ ID NO. 104278, or a homolog or fragment thereof, or if the activity of a polypeptide encoded by a nucleic acid molecule comprising the nucleic acid SEQ ID NO. 104277, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 104277 or polypeptide SEQ ID NO. 104278, respectively, or a homolog or a fragment thereof, is increased or generated, or if the activity GDP-mannose 4,6-dehydratase is increased or generated in a non-human organism, like a microorganism or a plant cell, plant or part thereof, especially with non-targeted localization, whereby the respective line disclose in column 7 the fine chemical fucose. For example, an increase of the fucose of at least 1 percent, particularly in a range of 1022 to 6264-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a transport protein, or if the activity of the polypeptide B2063, preferably represented by SEQ ID NO. 38948, or a homolog or fragment thereof, or if the activity of a polypeptide encoded by a nucleic acid molecule comprising the nucleic acid SEQ ID NO. 38947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 38947 or polypeptide SEQ ID NO. 38948, respectively, or a homolog or a fragment thereof, is increased or generated, or if the activity transport protein is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 78 to 1849-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a transport protein, or if the activity of the polypeptide B2063, preferably represented by SEQ ID NO. 38948, or a homolog or fragment thereof, or if the activity of a polypeptide encoded by a nucleic acid molecule comprising the nucleic acid SEQ ID NO. 38947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 38947 or polypeptide SEQ ID NO. 38948, respectively, or a homolog or a fragment thereof, is increased or generated, or if the activity transport protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 32 to 87-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a transport protein, or if the activity of the polypeptide B2063, preferably represented by SEQ ID NO. 38948, or a homolog or fragment thereof, or if the activity of a polypeptide encoded by a nucleic acid molecule comprising the nucleic acid SEQ ID NO. 38947, preferably the coding region thereof, or a homolog or fragment thereof, e.g. derived from Escherichia coli, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 38947 or polypeptide SEQ ID NO. 38948, respectively, or a homolog or a fragment thereof, is increased or generated, or if the activity transport protein is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 59 to 242-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a transport protein, or if the activity of the polypeptide B2063, preferably represented by SEQ ID NO. 38948, or a homolog or fragment thereof, or if the activity of a polypeptide encoded by a nucleic acid molecule comprising the nucleic acid SEQ ID NO. 38947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 38947 or polypeptide SEQ ID NO. 38948, respectively, or a homolog or a fragment thereof, is increased or generated, or if the activity transport protein is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 24 to 87-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a transport protein, or if the activity of the polypeptide B2063, preferably represented by SEQ ID NO. 38948, or a homolog or fragment thereof, or if the activity of a polypeptide encoded by a nucleic acid molecule comprising the nucleic acid SEQ ID NO. 38947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 38947 or polypeptide SEQ ID NO. 38948, respectively, or a homolog or a fragment thereof, is increased or generated, or if the activity transport protein is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 37 to 111-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a b2107-protein, or if the activity of the polypeptide B2107, preferably represented by SEQ ID NO. 39003, or a homolog or fragment thereof, or if the activity of a polypeptide encoded by a nucleic acid molecule comprising the nucleic acid SEQ ID NO. 39002, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 39002 or polypeptide SEQ ID NO. 39003, respectively, or a homolog or a fragment thereof, is increased or generated, or if the activity b2107-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 37 to 71-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a b2107-protein, or if the activity of the polypeptide B2107, preferably represented by SEQ ID NO. 39003, or a homolog or fragment thereof, or if the activity of a polypeptide encoded by a nucleic acid molecule comprising the nucleic acid SEQ ID NO. 39002, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 39002 or polypeptide SEQ ID NO. 39003, respectively, or a homolog or a fragment thereof, is increased or generated, or if the activity b2107-protein is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 39 to 94-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a b2139-protein, or if the activity of the polypeptide B2139, preferably represented by SEQ ID NO. 125086, or a homolog or fragment thereof, or if the activity of a polypeptide encoded by a nucleic acid molecule comprising the nucleic acid SEQ ID NO. 125085, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 125085 or polypeptide SEQ ID NO. 125086, respectively, or a homolog or a fragment thereof, is increased or generated, or if the activity b2139-protein is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 32 to 44-percent is conferred as compared to a corresponding non-transformed wild type non-human organism.

Accordingly, in one embodiment, an increase of erythrol in a non-human organism, as compared to a corresponding non-transformed wild type non-human organism, is conferred in the process of the invention, if the activity of a polypeptide showing the activity of a sn-glycerol-3-phosphate dehydrogenase, or if the activity of the polypeptide B2242, preferably represented by SEQ ID NO. 79280, or a homolog or fragment thereof, or if the activity of a polypeptide encoded by a nucleic acid molecule comprising the nucleic acid SEQ ID NO. 79279, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 79279 or polypeptide SEQ ID NO. 79280, respectively, or a homolog or a fragment thereof, is increased or generated, or if the activity sn-glycerol-3-phosphate dehydrogenase is increased or generated in a non-human organism, like a microorganism or a plant cell, plant or part thereof, especially with plastidic localization, whereby the respective line disclose in column 7 the fine chemical erythrol. For example, an increase of the erythrol of at least 1 percent, particularly in a range of 244 to 808-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a ATP synthase subunit E, or if the activity of the polypeptide B2285, preferably represented by SEQ ID NO. 99621, or a homolog or fragment thereof, or if the activity of a polypeptide encoded by a nucleic acid molecule comprising the nucleic acid SEQ ID NO. 99620, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 99620 or polypeptide SEQ ID NO. 99621, respectively, or a homolog or a fragment thereof, is increased or generated, or if the activity ATP synthase subunit E is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 26 to 113-percent is conferred as compared to a corresponding non-transformed wild type non-human organism.

Accordingly, in one embodiment, an increase of galactinol in a non-human organism, as compared to a corresponding non-transformed wild type non-human organism, is conferred in the process of the invention, if the activity of a polypeptide showing the activity of a aminotransferase, or if the activity of the polypeptide B2290, preferably represented by SEQ ID NO. 145693, or a homolog or fragment thereof, or if the activity of a polypeptide encoded by a nucleic acid molecule comprising the nucleic acid SEQ ID NO. 145692, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 145692 or polypeptide SEQ ID NO. 145693, respectively, or a homolog or a fragment thereof, is increased or generated, or if the activity aminotransferase is increased or generated in a non-human organism, like a microorganism or a plant cell, plant or part thereof, especially with non-targeted localization, whereby the respective line disclose in column 7 the fine chemical galactinol. For example, an increase of the galactinol of at least 1 percent, particularly in a range of 54 to 149-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a b2345-protein, or if the activity of the polypeptide B2345, preferably represented by SEQ ID NO. 9157, or a homolog or fragment thereof, or if the activity of a polypeptide encoded by a nucleic acid molecule comprising the nucleic acid SEQ ID NO. 9156, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 9156 or polypeptide SEQ ID NO. 9157, respectively, or a homolog or a fragment thereof, is increased or generated, or if the activity b2345-protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 25 to 60-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a b2345-protein, or if the activity of the polypeptide B2345, preferably represented by SEQ ID NO. 9157, or a homolog or fragment thereof, or if the activity of a polypeptide encoded by a nucleic acid molecule comprising the nucleic acid SEQ ID NO. 9156, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 9156 or polypeptide SEQ ID NO. 9157, respectively, or a homolog or a fragment thereof, is increased or generated, or if the activity b2345-protein is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 41 to 68-percent is conferred as compared to a corresponding non-transformed wild type non-human organism.

Accordingly, in one embodiment, an increase of verbascose in a non-human organism, as compared to a corresponding non-transformed wild type non-human organism, is conferred in the process of the invention, if the activity of a polypeptide showing the activity of a b2360-protein, or if the activity of the polypeptide B2360, preferably represented by SEQ ID NO. 39220, or a homolog or fragment thereof, or if the activity of a polypeptide encoded by a nucleic acid molecule comprising the nucleic acid SEQ ID NO. 39219, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 39219 or polypeptide SEQ ID NO. 39220, respectively, or a homolog or a fragment thereof, is increased or generated, or if the activity b2360-protein is increased or generated in a non-human organism, like a microorganism or a plant cell, plant or part thereof, especially with non-targeted localization, whereby the respective line disclose in column 7 the fine chemical verbascose. For example, an increase of the verbascose of at least 1 percent, particularly in a range of 67 to 1435-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a b2360-protein, or if the activity of the polypeptide B2360, preferably represented by SEQ ID NO. 39220, or a homolog or fragment thereof, or if the activity of a polypeptide encoded by a nucleic acid molecule comprising the nucleic acid SEQ ID NO. 39219, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 39219 or polypeptide SEQ ID NO. 39220, respectively, or a homolog or a fragment thereof, is increased or generated, or if the activity b2360-protein is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 38 to 65-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a b2361-protein, or if the activity of the polypeptide B2361, preferably represented by SEQ ID NO. 146205, or a homolog or fragment thereof, or if the activity of a polypeptide encoded by a nucleic acid molecule comprising the nucleic acid SEQ ID NO. 146204, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 146204 or polypeptide SEQ ID NO. 146205, respectively, or a homolog or a fragment thereof, is increased or generated, or if the activity b2361-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 31 to 46-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a B2361-protein, or if the activity of the polypeptide B2361, preferably represented by SEQ ID NO. 146205, or a homolog or fragment thereof, or if the activity of a polypeptide encoded by a nucleic acid molecule comprising the nucleic acid SEQ ID NO. 146204, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 146204 or polypeptide SEQ ID NO. 146205, respectively, or a homolog or a fragment thereof, is increased or generated, or if the activity b2361-protein is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 33 to 79-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a ion-transport protein, or if the activity of the polypeptide B2389, preferably represented by SEQ ID NO. 146239, or a homolog or fragment thereof, or if the activity of a polypeptide encoded by a nucleic acid molecule comprising the nucleic acid SEQ ID NO. 146238, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 146238 or polypeptide SEQ ID NO. 146239, respectively, or a homolog or a fragment thereof, is increased or generated, or if the activity ion-transport protein is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 32 to 83-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a b2399-protein, or if the activity of the polypeptide B2399, preferably represented by SEQ ID NO. 39238, or a homolog or fragment thereof, or if the activity of a polypeptide encoded by a nucleic acid molecule comprising the nucleic acid SEQ ID NO. 39237, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 39237 or polypeptide SEQ ID NO. 39238, respectively, or a homolog or a fragment thereof, is increased or generated, or if the activity b2399-protein is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 147 to 1537-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a b2399-protein, or if the activity of the polypeptide B2399, preferably represented by SEQ ID NO. 39238, or a homolog or fragment thereof, or if the activity of a polypeptide encoded by a nucleic acid molecule comprising the nucleic acid SEQ ID NO. 39237, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 39237 or polypeptide SEQ ID NO. 39238, respectively, or a homolog or a fragment thereof, is increased or generated, or if the activity b2399-protein is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 97 to 2165-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a b2399-protein, or if the activity of the polypeptide B2399, preferably represented by SEQ ID NO. 39238, or a homolog or fragment thereof, or if the activity of a polypeptide encoded by a nucleic acid molecule comprising the nucleic acid SEQ ID NO. 39237, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 39237 or polypeptide SEQ ID NO. 39238, respectively, or a homolog or a fragment thereof, is increased or generated, or if the activity b2399-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 68 to 231-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a b2399-protein, or if the activity of the polypeptide B2399, preferably represented by SEQ ID NO. 39238, or a homolog or fragment thereof, or if the activity of a polypeptide encoded by a nucleic acid molecule comprising the nucleic acid SEQ ID NO. 39237, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 39237 or polypeptide SEQ ID NO. 39238, respectively, or a homolog or a fragment thereof, is increased or generated, or if the activity b2399-protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 33 to 1261-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a b2399-protein, or if the activity of the polypeptide B2399, preferably represented by SEQ ID NO. 39238, or a homolog or fragment thereof, or if the activity of a polypeptide encoded by a nucleic acid molecule comprising the nucleic acid SEQ ID NO. 39237, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 39237 or polypeptide SEQ ID NO. 39238, respectively, or a homolog or a fragment thereof, is increased or generated, or if the activity b2399-protein is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 862 to 3481-percent is conferred as compared to a corresponding non-transformed wild type non-human organism.

Accordingly, in one embodiment, an increase of sedoheptulose-7-phosphate in a non-human organism, as compared to a corresponding non-transformed wild type non-human organism, is conferred in the process of the invention, if the activity of a polypeptide showing the activity of a b2399-protein, or if the activity of the polypeptide B2399, preferably represented by SEQ ID NO. 39238, or a homolog or fragment thereof, or if the activity of a polypeptide encoded by a nucleic acid molecule comprising the nucleic acid SEQ ID NO. 39237, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 39237 or polypeptide SEQ ID NO. 39238, respectively, or a homolog or a fragment thereof, is increased or generated, or if the activity b2399-protein is increased or generated in a non-human organism, like a microorganism or a plant cell, plant or part thereof, especially with non-targeted localization, whereby the respective line disclose in column 7 the fine chemical sedoheptulose-7-phosphate. For example, an increase of the sedoheptulose-7-phosphate of at least 1 percent, particularly in a range of 51 to 583-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a b2399-protein, or if the activity of the polypeptide B2399, preferably represented by SEQ ID NO. 39238, or a homolog or fragment thereof, or if the activity of a polypeptide encoded by a nucleic acid molecule comprising the nucleic acid SEQ ID NO. 39237, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 39237 or polypeptide SEQ ID NO. 39238, respectively, or a homolog or a fragment thereof, is increased or generated, or if the activity b2399-protein is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 26 to 131-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a transcriptional regulator, or if the activity of the polypeptide B2405, preferably represented by SEQ ID NO. 39256, or a homolog or fragment thereof, or if the activity of a polypeptide encoded by a nucleic acid molecule comprising the nucleic acid SEQ ID NO. 39255, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 39255 or polypeptide SEQ ID NO. 39256, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 77 to 106-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a transcriptional regulator, or if the activity of the polypeptide B2405, preferably represented by SEQ ID NO. 39256, or a homolog or fragment thereof, or if the activity of a polypeptide encoded by a nucleic acid molecule comprising the nucleic acid SEQ ID NO. 39255, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 39255 or polypeptide SEQ ID NO. 39256, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 58 to 61-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a cysteine synthase A, or if the activity of the polypeptide B2414, preferably represented by SEQ ID NO. 39301, or a homolog or fragment thereof, or if the activity of a polypeptide encoded by a nucleic acid molecule comprising the nucleic acid SEQ ID NO. 39300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 39300 or polypeptide SEQ ID NO. 39301, respectively, or a homolog or a fragment thereof, is increased or generated, or if the activity cysteine synthase A is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 26 to 55-percent is conferred as compared to a corresponding non-transformed wild type non-human organism.

Accordingly, in one embodiment, an increase of verbascose in a non-human organism, as compared to a corresponding non-transformed wild type non-human organism, is conferred in the process of the invention, if the activity of a polypeptide showing the activity of a cysteine synthase A, or if the activity of the polypeptide B2414, preferably represented by SEQ ID NO. 39301, or a homolog or fragment thereof, or if the activity of a polypeptide encoded by a nucleic acid molecule comprising the nucleic acid SEQ ID NO. 39300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 39300 or polypeptide SEQ ID NO. 39301, respectively, or a homolog or a fragment thereof, is increased or generated, or if the activity cysteine synthase A is increased or generated in a non-human organism, like a microorganism or a plant cell, plant or part thereof, especially with non-targeted localization, whereby the respective line disclose in column 7 the fine chemical verbascose. For example, an increase of the verbascose of at least 1 percent, particularly in a range of 49 to 743-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a b2466-protein, or if the activity of the polypeptide B2466, preferably represented by SEQ ID NO. 146262, or a homolog or fragment thereof, or if the activity of a polypeptide encoded by a nucleic acid molecule comprising the nucleic acid SEQ ID NO. 146261, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 146261 or polypeptide SEQ ID NO. 146262, respectively, or a homolog or a fragment thereof, is increased or generated, or if the activity b2466-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 35 to 116-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a b2466-protein, or if the activity of the polypeptide B2466, preferably represented by SEQ ID NO. 146262, or a homolog or fragment thereof, or if the activity of a polypeptide encoded by a nucleic acid molecule comprising the nucleic acid SEQ ID NO. 146261, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 146261 or polypeptide SEQ ID NO. 146262, respectively, or a homolog or a fragment thereof, is increased or generated, or if the activity b2466-protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 33 to 154-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a b2466-protein, or if the activity of the polypeptide B2466, preferably represented by SEQ ID NO. 146262, or a homolog or fragment thereof, or if the activity of a polypeptide encoded by a nucleic acid molecule comprising the nucleic acid SEQ ID NO. 146261, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 146261 or polypeptide SEQ ID NO. 146262, respectively, or a homolog or a fragment thereof, is increased or generated, or if the activity b2466-protein is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, partitularly in a range of 37 to 62-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a b2474-protein, or if the activity of the polypeptide B2474, preferably represented by SEQ ID NO. 40330, or a homolog or fragment thereof, or if the activity of a polypeptide encoded by a nucleic acid molecule comprising the nucleic acid SEQ ID NO. 40329, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 40329 or polypeptide SEQ ID NO. 40330, respectively, or a homolog or a fragment thereof, is increased or generated, or if the activity b2474-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 23 to 57-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a 2,3-dihydroxy-2,3-dihydrophenylpropionatedehydrogenase, or if the activity of the polypeptide B2541, preferably represented by SEQ ID NO. 40384, or a homolog or fragment thereof, or if the activity of a polypeptide encoded by a nucleic acid molecule comprising the nucleic acid SEQ ID NO. 40383, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 40383 or polypeptide SEQ ID NO. 40384, respectively, or a homolog or a fragment thereof, is increased or generated, or if the activity 2,3-dihydroxy-2,3-dihydrophenylpropionatedehydrogenase is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 95 to 173-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a putative transport system permease protein, or if the activity of the polypeptide B2546, preferably represented by SEQ ID NO. 99899, or a homolog or fragment thereof, or if the activity of a polypeptide encoded by a nucleic acid molecule comprising the nucleic acid SEQ ID NO. 99898, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 99898 or polypeptide SEQ ID NO. 99899, respectively, or a homolog or a fragment thereof, is increased or generated, or if the activity putative transport system permease protein is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 23 to 38-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a b2613-protein, or if the activity of the polypeptide B2613, preferably represented by SEQ ID NO. 40666, or a homolog or fragment thereof, or if the activity of a polypeptide encoded by a nucleic acid molecule comprising the nucleic acid SEQ ID NO. 40665, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 40665 or polypeptide SEQ ID NO. 40666, respectively, or a homolog or a fragment thereof, is increased or generated, or if the activity b2613-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 18 to 42-percent is conferred as compared to a corresponding non-transformed wild type non-human organism

Accordingly, in one embodiment, an increase of myo-inositol in a non-human organism, as compared to a corresponding non-transformed wild type non-human organism, is conferred in the process of the invention, if the activity of a polypeptide showing the activity of a b2613-protein, or if the activity of the polypeptide B2613, preferably represented by SEQ ID NO. 40666, or a homolog or fragment thereof, or if the activity of a polypeptide encoded by a nucleic acid molecule comprising the nucleic acid SEQ ID NO. 40665, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 40665 or polypeptide SEQ ID NO. 40666, respectively, or a homolog or a fragment thereof, is increased or generated, or if the activity b2613-protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 26 to 43-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a transcriptional regulator, or if the activity of the polypeptide B2634, preferably represented by SEQ ID NO. 40727, or a homolog or fragment thereof, or if the activity of a polypeptide encoded by a nucleic acid molecule comprising the nucleic acid SEQ ID NO. 40726, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 40726 or polypeptide SEQ ID NO. 40727, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 110 to 4796-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a transcriptional regulator, or if the activity of the polypeptide B2634, preferably represented by SEQ ID NO. 40727, or a homolog or fragment thereof, or if the activity of a polypeptide encoded by a nucleic acid molecule comprising the nucleic acid SEQ ID NO. 40726, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 40726 or polypeptide SEQ ID NO. 40727, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 27 to 1006-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a transcriptional regulator, or if the activity of the polypeptide B2634, preferably represented by SEQ ID NO. 40727, or a homolog or fragment thereof, or if the activity of a polypeptide encoded by a nucleic acid molecule comprising the nucleic acid SEQ ID NO. 40726, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 40726 or polypeptide SEQ ID NO. 40727, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 28 to 696-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a b2653-protein, or if the activity of the polypeptide B2653, preferably represented by SEQ ID NO. 146289, or a homolog or fragment thereof, or if the activity of a polypeptide encoded by a nucleic acid molecule comprising the nucleic acid SEQ ID NO. 146288, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 146288 or polypeptide SEQ ID NO. 146289, respectively, or a homolog or a fragment thereof, is increased or generated, or if the activity b2653-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 31 to 45-percent is conferred as compared to a corresponding non-transformed wild type non-human organism.

Accordingly, in one embodiment, an increase of sedoheptulose-7-phosphate in a non-human organism, as compared to a corresponding non-transformed wild type non-human organism, is conferred in the process of the invention, if the activity of a polypeptide showing the activity of a b2653-protein, or if the activity of the polypeptide B2653, preferably represented by SEQ ID NO. 146289, or a homolog or fragment thereof, or if the activity of a polypeptide encoded by a nucleic acid molecule comprising the nucleic acid SEQ ID NO. 146288, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 146288 or polypeptide SEQ ID NO. 146289, respectively, or a homolog or a fragment thereof, is increased or generated, or if the activity b2653-protein is increased or generated in a non-human organism, like a microorganism or a plant cell, plant or part thereof, especially with non-targeted localization, whereby the respective line disclose in column 7 the fine chemical sedoheptulose-7-phosphate. For example, an increase of the sedoheptulose-7-phosphate of at least 1 percent, particularly in a range of 57 to 70-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a b2653-protein, or if the activity of the polypeptide B2653, preferably represented by SEQ ID NO. 146289, or a homolog or fragment thereof, or if the activity of a polypeptide encoded by a nucleic acid molecule comprising the nucleic acid SEQ ID NO. 146288, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 146288 or polypeptide SEQ ID NO. 146289, respectively, or a homolog or a fragment thereof, is increased or generated, or if the activity b2653-protein is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 37 to 69-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a b2654-protein, or if the activity of the polypeptide B2654, preferably represented by SEQ ID NO. 125227, or a homolog or fragment thereof, or if the activity of a polypeptide encoded by a nucleic acid molecule comprising the nucleic acid SEQ ID NO. 125226, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 125226 or polypeptide SEQ ID NO. 125227, respectively, or a homolog or a fragment thereof, is increased or generated, or if the activity b2654-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 39 to 54-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a b2654-protein, or if the activity of the polypeptide B2654, preferably represented by SEQ ID NO. 125227, or a homolog or fragment thereof, or if the activity of a polypeptide encoded by a nucleic acid molecule comprising the nucleic acid SEQ ID NO. 125226, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 125226 or polypeptide SEQ ID NO. 125227, respectively, or a homolog or a fragment thereof, is increased or generated, or if the activity b2654-protein is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 34 to 70-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a b2670-protein, or if the activity of the polypeptide B2670, preferably represented by SEQ ID NO. 146295, or a homolog or fragment thereof, or if the activity of a polypeptide encoded by a nucleic acid molecule comprising the nucleic acid SEQ ID NO. 146294, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 146294 or polypeptide SEQ ID NO. 146295, respectively, or a homolog or a fragment thereof, is increased or generated, or if the activity b2670-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 34 to 97-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a b2673-protein, or if the activity of the polypeptide B2673, preferably represented by SEQ ID NO. 9245, or a homolog or fragment thereof, or if the activity of a polypeptide encoded by a nucleic acid molecule comprising the nucleic acid SEQ ID NO. 9244, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 9244 or polypeptide SEQ ID NO. 9245, respectively, or a homolog or a fragment thereof, is increased or generated, or if the activity b2673-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 31 to 60-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a b2673-protein, or if the activity of the polypeptide B2673, preferably represented by SEQ ID NO. 9245, or a homolog or fragment thereof, or if the activity of a polypeptide encoded by a nucleic acid molecule comprising the nucleic acid SEQ ID NO. 9244, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 9244 or polypeptide SEQ ID NO. 9245, respectively, or a homolog or a fragment thereof, is increased or generated, or if the activity b2673-protein is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 40 to 56-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a b2682-protein, or if the activity of the polypeptide B2682, preferably represented by SEQ ID NO. 115656, or a homolog or fragment thereof, or if the activity of a polypeptide encoded by a nucleic acid molecule comprising the nucleic acid SEQ ID NO. 115655, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 115655 or polypeptide SEQ ID NO. 115656, respectively, or a homolog or a fragment thereof, is increased or generated, or if the activity b2682-protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 25 to 113-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a recombinase A, or if the activity of the polypeptide B2699, preferably represented by SEQ ID NO. 100106, or a homolog or fragment thereof, or if the activity of a polypeptide encoded by a nucleic acid molecule comprising the nucleic acid SEQ ID NO. 100105, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 100105 or polypeptide SEQ ID NO. 100106, respectively, or a homolog or a fragment thereof, is increased or generated, or if the activity recombinase A is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 65 to 214-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a murein transglycosylase, or if the activity of the polypeptide B2701, preferably represented by SEQ ID NO. 80757, or a homolog or fragment thereof, or if the activity of a polypeptide encoded by a nucleic acid molecule comprising the nucleic acid SEQ ID NO. 80756, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 80756 or polypeptide SEQ ID NO. 80757, respectively, or a homolog or a fragment thereof, is increased or generated, or if the activity murein transglycosylase is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 36 to 59-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a transcriptional regulator, or if the activity of the polypeptide B2714, preferably represented by SEQ ID NO. 40742, or a homolog or fragment thereof, or if the activity of a polypeptide encoded by a nucleic acid molecule comprising the nucleic acid SEQ ID NO. 40741, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 40741 or polypeptide SEQ ID NO. 40742, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 29 to 45-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a b2739-protein, or if the activity of the polypeptide B2739, preferably represented by SEQ ID NO. 80907, or a homolog or fragment thereof, or if the activity of a polypeptide encoded by a nucleic acid molecule comprising the nucleic acid SEQ ID NO. 80906, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 80906 or polypeptide SEQ ID NO. 80907, respectively, or a homolog or a fragment thereof, is increased or generated, or if the activity b2739-protein is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 76 to 544-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a b2739-protein, or if the activity of the polypeptide B2739, preferably represented by SEQ ID NO. 80907, or a homolog or fragment thereof, or if the activity of a polypeptide encoded by a nucleic acid molecule comprising the nucleic acid SEQ ID NO. 80906, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 80906 or polypeptide SEQ ID NO. 80907, respectively, or a homolog or a fragment thereof, is increased or generated, or if the activity b2739-protein is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 77 to 212-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a 2-C-methyl-D-erythritol 4-phosphate cytidylyltransferase, or if the activity of the polypeptide B2747, preferably represented by SEQ ID NO. 81076, or a homolog or fragment thereof, or if the activity of a polypeptide encoded by a nucleic acid molecule comprising the nucleic acid SEQ ID NO. 81075, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 81075 or polypeptide SEQ ID NO. 81076, respectively, or a homolog or a fragment thereof, is increased or generated, or if the activity 2-C-methyl-D-erythritol 4-phosphate cytidylyltransferase is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 81 to 417-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a 2-C-methyl-D-erythritol 4-phosphate cytidylyltransferase, or if the activity of the polypeptide B2747, preferably represented by SEQ ID NO. 81076, or a homolog or fragment thereof, or if the activity of a polypeptide encoded by a nucleic acid molecule comprising the nucleic acid SEQ ID NO. 81075, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 81075 or polypeptide SEQ ID NO. 81076, respectively, or a homolog or a fragment thereof, is increased or generated, or if the activity 2-C-methyl-D-erythritol 4-phosphate cytidylyltransferase is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1-percent, particularly in a range of 34 to 238-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a 2-C-methyl-D-erythritol 4-phosphate cytidylyltransferase, or if the activity of the polypeptide B2747, preferably represented by SEQ ID NO. 81076, or a homolog or fragment thereof, or if the activity of a polypeptide encoded by a nucleic acid molecule comprising the nucleic acid SEQ ID NO. 81075, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 81075 or polypeptide SEQ ID NO. 81076, respectively, or a homolog or a fragment thereof, is increased or generated, or if the activity 2-C-methyl-D-erythritol 4-phosphate cytidylyltransferase is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 64 to 612-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a phosphoadenosine phosphosulfate reductase, or if the activity of the polypeptide B2762, preferably represented by SEQ ID NO. 81452, or a homolog or fragment thereof, or if the activity of a polypeptide encoded by a nucleic acid molecule comprising the nucleic acid SEQ ID NO. 81451, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 81451 or polypeptide SEQ ID NO. 81452, respectively, or a homolog or a fragment thereof, is increased or generated, or if the activity phosphoadenosine phosphosulfate reductase is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 18 to 30-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a phosphoadenosine phosphosulfate reductase, or if the activity of the polypeptide B2762, preferably represented by SEQ ID NO. 81452, or a homolog or fragment thereof, or if the activity of a polypeptide encoded by a nucleic acid molecule comprising the nucleic acid SEQ ID NO. 81451, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 81451 or polypeptide SEQ ID NO. 81452, respectively, or a homolog or a fragment thereof, is increased or generated, or if the activity phosphoadenosine phosphosulfate reductase is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 35 to 58-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a flavodoxin, or if the activity of the polypeptide B2790, preferably represented by SEQ ID NO. 115720, or a homolog or fragment thereof, or if the activity of a polypeptide encoded by a nucleic acid molecule comprising the nucleic acid SEQ ID NO. 115719, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 115719 or polypeptide SEQ ID NO. 115720, respectively, or a homolog or a fragment thereof, is increased or generated, or if the activity flavodoxin is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 134 to 138-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a flavodoxin, or if the activity of the polypeptide B2790, preferably represented by SEQ ID NO. 115720, or a homolog or fragment thereof, or if the activity of a polypeptide encoded by a nucleic acid molecule comprising the nucleic acid SEQ ID NO. 115719, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 115719 or polypeptide SEQ ID NO. 115720, respectively, or a homolog or a fragment thereof, is increased or generated, or if the activity flavodoxin is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 60 to 155-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a flavodoxin, or if the activity of the polypeptide B2790, preferably represented by SEQ ID NO. 115720, or a homolog or fragment thereof, or if the activity of a polypeptide encoded by a nucleic acid molecule comprising the nucleic acid SEQ ID NO. 115719, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 115719 or polypeptide SEQ ID NO. 115720, respectively, or a homolog or a fragment thereof, is increased or generated, or if the activity flavodoxin is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 32 to 81-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a flavodoxin, or if the activity of the polypeptide B2790, preferably represented by SEQ ID NO. 115720, or a homolog or fragment thereof, or if the activity of a polypeptide encoded by a nucleic acid molecule comprising the nucleic acid SEQ ID NO. 115719, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 115719 or polypeptide SEQ ID NO. 115720, respectively, or a homolog or a fragment thereof, is increased or generated, or if the activity flavodoxin is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 25 to 116-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a flavodoxin, or if the activity of the polypeptide B2790, preferably represented by SEQ ID NO. 115720, or a homolog or fragment thereof, or if the activity of a polypeptide encoded by a nucleic acid molecule comprising the nucleic acid SEQ ID NO. 115719, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 115719 or polypeptide SEQ ID NO. 115720, respectively, or a homolog or a fragment thereof, is increased or generated, or if the activity flavodoxin is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 43 to 90-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a amino-acid acetyltransferase, or if the activity of the polypeptide B2818, preferably represented by SEQ ID NO. 81577, or a homolog or fragment thereof, or if the activity of a polypeptide encoded by a nucleic acid molecule comprising the nucleic acid SEQ ID NO. 81576, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 81576 or polypeptide SEQ ID NO. 81577, respectively, or a homolog or a fragment thereof, is increased or generated, or if the activity amino-acid acetyltransferase is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 89 to 1378-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a amino-acid acetyltransferase, or if the activity of the polypeptide B2818, preferably represented by SEQ ID NO. 81577, or a homolog or fragment thereof, or if the activity of a polypeptide encoded by a nucleic acid molecule comprising the nucleic acid SEQ ID NO. 81576, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 81576 or polypeptide SEQ ID NO. 81577, respectively, or a homolog or a fragment thereof, is increased or generated, or if the activity amino-acid acetyltransferase is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 25 to 43-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a amino-acid acetyltransferase, or if the activity of the polypeptide B2818, preferably represented by SEQ ID NO. 81577, or a homolog or fragment thereof, or if the activity of a polypeptide encoded by a nucleic acid molecule comprising the nucleic acid SEQ ID NO. 81576, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 81576 or polypeptide SEQ ID NO. 81577, respectively, or a homolog or a fragment thereof, is increased or generated, or if the activity amino-acid acetyltransferase is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 25 to 67-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a transcriptional regulator, or if the activity of the polypeptide B2839, preferably represented by SEQ ID NO. 81936, or a homolog or fragment thereof, or if the activity of a polypeptide encoded by a nucleic acid molecule comprising the nucleic acid SEQ ID NO. 81935, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 81935 or polypeptide SEQ ID NO. 81936, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 121 to 159-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a arginine exporter protein, or if the activity of the polypeptide B2923, preferably represented by SEQ ID NO. 9334, or a homolog or fragment thereof, or if the activity of a polypeptide encoded by a nucleic acid molecule comprising the nucleic acid SEQ ID NO. 9333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 9333 or polypeptide SEQ ID NO. 9334, respectively, or a homolog or a fragment thereof, is increased or generated, or if the activity arginine exporter protein is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 77 to 194-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a arginine exporter protein, or if the activity of the polypeptide B2923, preferably represented by SEQ ID NO. 9334, or a homolog or fragment thereof, or if the activity of a polypeptide encoded by a nucleic acid molecule comprising the nucleic acid SEQ ID NO. 9333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 9333 or polypeptide SEQ ID NO. 9334, respectively, or a homolog or a fragment thereof, is increased or generated, or if the activity arginine exporter protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 24 to 107-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a b2932-protein, or if the activity of the polypeptide B2932, preferably represented by SEQ ID NO. 146328, or a homolog or fragment thereof, or if the activity of a polypeptide encoded by a nucleic acid molecule comprising the nucleic acid SEQ ID NO. 146327, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 146327 or polypeptide SEQ ID NO. 146328, respectively, or a homolog or a fragment thereof, is increased or generated, or if the activity b2932-protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 26 to 61-percent is conferred as compared to a corresponding non-transformed wild type non-human organism.

Accordingly, in one embodiment, an increase of erythrol in a non-human organism, as compared to a corresponding non-transformed wild type non-human organism, is conferred in the process of the invention, if the activity of a polypeptide showing the activity of a L-serine dehydratase, or if the activity of the polypeptide B3112, preferably represented by SEQ ID NO. 104661, or a homolog or fragment thereof, or if the activity of a polypeptide encoded by a nucleic acid molecule comprising the nucleic acid SEQ ID NO. 104660, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 104660 or polypeptide SEQ ID NO. 104661, respectively, or a homolog or a fragment thereof, is increased or generated, or if the activity L-serine dehydratase is increased or generated in a non-human organism, like a microorganism or a plant cell, plant or part thereof, especially with plastidic localization, whereby the respective line disclose in column 7 the fine chemical erythrol. For example, an increase of the erythrol of at least 1 percent, particularly in a range of 147 to 413-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a b3151-protein, or if the activity of the polypeptide B3151, preferably represented by SEQ ID NO. 42478, or a homolog or fragment thereof, or if the activity of a polypeptide encoded by a nucleic acid molecule comprising the nucleic acid SEQ ID NO. 42477, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 42477 or polypeptide SEQ ID NO. 42478, respectively, or a homolog or a fragment thereof, is increased or generated, or if the activity b3151-protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 33 to 139-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a b3151-protein, or if the activity of the polypeptide B3151, preferably represented by SEQ ID NO. 42478, or a homolog or fragment thereof, or if the activity of a polypeptide encoded by a nucleic acid molecule comprising the nucleic acid SEQ ID NO. 42477, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 42477 or polypeptide SEQ ID NO. 42478, respectively, or a homolog or a fragment thereof, is increased or generated, or if the activity b3151-protein is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 65 to 188-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a b3151-protein, or if the activity of the polypeptide B3151, preferably represented by SEQ ID NO. 42478, or a homolog or fragment thereof, or if the activity of a polypeptide encoded by a nucleic acid molecule comprising the nucleic acid SEQ ID NO. 42477, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 42477 or polypeptide SEQ ID NO. 42478, respectively, or a homolog or a fragment thereof, is increased or generated, or if the activity b3151-protein is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 25 to 64-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a b3221-protein, or if the activity of the polypeptide B3221, preferably represented by SEQ ID NO. 82511, or a homolog or fragment thereof, or if the activity of a polypeptide encoded by a nucleic acid molecule comprising the nucleic acid SEQ ID NO. 82510, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 82510 or polypeptide SEQ ID NO. 82511, respectively, or a homolog or a fragment thereof, is increased or generated, or if the activity b3221-protein is increased or generated in a non-human organism, like a microorganism or a plant cell, plant or part thereof, especially with plastidic localization, whereby the respective line disclose in column 7 the fine chemical fructose. For example, an increase of the fructose of at least 1 percent, particularly in a range of 40 to 58-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a b3221-protein, or if the activity of the polypeptide B3221, preferably represented by SEQ ID NO. 82511, or a homolog or fragment thereof, or if the activity of a polypeptide encoded by a nucleic acid molecule comprising the nucleic acid SEQ ID NO. 82510, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 82510 or polypeptide SEQ ID NO. 82511, respectively, or a homolog or a fragment thereof, is increased or generated, or if the activity b3221-protein is increased or generated in a non-human organism, like a microorganism or a plant cell, plant or part thereof, especially with plastidic localization, whereby the respective line disclose in column 7 the fine chemical glucose. For example, an increase of the glucose of at least 1 percent, particularly in a range of 58 to 73-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a b3221-protein, or if the activity of the polypeptide B3221, preferably represented by SEQ ID NO. 82511, or a homolog or fragment thereof, or if the activity of a polypeptide encoded by a nucleic acid molecule comprising the nucleic acid SEQ ID NO. 82510, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 82510 or polypeptide SEQ ID NO. 82511, respectively, or a homolog or a fragment thereof, is increased or generated, or if the activity b3221-protein is increased or generated in a non-human organism, like a microorganism or a plant cell, plant or part thereof, especially with plastidic localization, whereby the respective line disclose in column 7 the fine chemical 3,4,5-trihydroxypentanoic acid. For example, an increase of the 3,4,5-trihydroxypentanoic acid of at least 1 percent, particularly in a range of 44 to 62-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a b3246-protein, or if the activity of the polypeptide B3246, preferably represented by SEQ ID NO. 9471, or a homolog or fragment thereof, or if the activity of a polypeptide encoded by a nucleic acid molecule comprising the nucleic acid SEQ ID NO. 9470, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 9470 or polypeptide SEQ ID NO. 9471, respectively, or a homolog or a fragment thereof, is increased or generated, or if the activity b3246-protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 311 to 476-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a acetyl CoA carboxylase, or if the activity of the polypeptide B3256, preferably represented by SEQ ID NO. 9493, or a homolog or fragment thereof, or if the activity of a polypeptide encoded by a nucleic acid molecule comprising the nucleic acid SEQ ID NO. 9492, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 9492 or polypeptide SEQ ID NO. 9493, respectively, or a homolog or a fragment thereof, is increased or generated, or if the activity acetyl CoA carboxylase is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 25 to 229-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a methyltransferase, or if the activity of the polypeptide B3262, preferably represented by SEQ ID NO. 42503, or a homolog or fragment thereof, or if the activity of a polypeptide encoded by a nucleic acid molecule comprising the nucleic acid SEQ ID NO. 42502, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 42502 or polypeptide SEQ ID NO. 42503, respectively, or a homolog or a fragment thereof, is increased or generated, or if the activity methyltransferase is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 79 to 736-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a methyltransferase, or if the activity of the polypeptide B3262, preferably represented by SEQ ID NO. 42503, or a homolog or fragment thereof, or if the activity of a polypeptide encoded by a nucleic acid molecule comprising the nucleic acid SEQ ID NO. 42502, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 42502 or polypeptide SEQ ID NO. 42503, respectively, or a homolog or a fragment thereof, is increased or generated, or if the activity methyltransferase is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 58 to 334-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a methyltransferase, or if the activity of the polypeptide B3262, preferably represented by SEQ ID NO. 42503, or a homolog or fragment thereof, or if the activity of a polypeptide encoded by a nucleic acid molecule comprising the nucleic acid SEQ ID NO. 42502, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 42502 or polypeptide SEQ ID NO. 42503, respectively, or a homolog or a fragment thereof, is increased or generated, or if the activity methyltransferase is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 43 to 64-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a methyltransferase, or if the activity of the polypeptide B3262, preferably represented by SEQ ID NO. 42503, or a homolog or fragment thereof, or if the activity of a polypeptide encoded by a nucleic acid molecule comprising the nucleic acid SEQ ID NO. 42502, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 42502 or polypeptide SEQ ID NO. 42503, respectively, or a homolog or a fragment thereof, is increased or generated, or if the activity methyltransferase is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 186 to 930-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a methyltransferase, or if the activity of the polypeptide B3262, preferably represented by SEQ ID NO. 42503, or a homolog or fragment thereof, or if the activity of a polypeptide encoded by a nucleic acid molecule comprising the nucleic acid SEQ ID NO. 42502, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 42502 or polypeptide SEQ ID NO. 42503, respectively, or a homolog or a fragment thereof, is increased or generated, or if the activity methyltransferase is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 30 to 67-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a ferredoxin, or if the activity of the polypeptide B3337, preferably represented by SEQ ID NO. 146365, or a homolog or fragment thereof, or if the activity of a polypeptide encoded by a nucleic acid molecule comprising the nucleic acid SEQ ID NO. 146364, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 146364 or polypeptide SEQ ID NO. 146365, respectively, or a homolog or a fragment thereof, is increased or generated, or if the activity ferredoxin is increased or generated in a non-human organism, like a microorganism or a plant cell, plant or part thereof, especially with plastidic localization, whereby the respective line disclose in column 7 the fine chemical glucose. For example, an increase of the glucose of at least 1 percent, particularly in a range of 38 to 48-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a b3346-protein, or if the activity of the polypeptide B3346, preferably represented by SEQ ID NO. 10105, or a homolog or fragment thereof, or if the activity of a polypeptide encoded by a nucleic acid molecule comprising the nucleic acid SEQ ID NO. 10104, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 10104 or polypeptide SEQ ID NO. 10105, respectively, or a homolog or a fragment thereof, is increased or generated, or if the activity b3346-protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 30 to 74-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a b3346-protein, or if the activity of the polypeptide B3346, preferably represented by SEQ ID NO. 10105, or a homolog or fragment thereof, or if the activity of a polypeptide encoded by a nucleic acid molecule comprising the nucleic acid SEQ ID NO. 10104, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 10104 or polypeptide SEQ ID NO. 10105, respectively, or a homolog or a fragment thereof, is increased or generated, or if the activity b3346-protein is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 73 to 370-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a b3346-protein, or if the activity of the polypeptide B3346, preferably represented by SEQ ID NO. 10105, or a homolog or fragment thereof, or if the activity of a polypeptide encoded by a nucleic acid molecule comprising the nucleic acid SEQ ID NO. 10104, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 10104 or polypeptide SEQ ID NO. 10105, respectively, or a homolog or a fragment thereof, is increased or generated, or if the activity b3346-protein is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 34 to 74-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a phosphoenolpyruvate carboxykinase, or if the activity of the polypeptide B3403, preferably represented by SEQ ID NO. 82867, or a homolog or fragment thereof, or if the activity of a polypeptide encoded by a nucleic acid molecule comprising the nucleic acid SEQ ID NO. 82866, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 82866 or polypeptide SEQ ID NO. 82867, respectively, or a homolog or a fragment thereof, is increased or generated, or if the activity phosphoenolpyruvate carboxykinase is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 77 to 143-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a phosphoenolpyruvate carboxykinase, or if the activity of the polypeptide B3403, preferably represented by SEQ ID NO. 82867, or a homolog or fragment thereof, or if the activity of a polypeptide encoded by a nucleic acid molecule comprising the nucleic acid SEQ ID NO. 82866, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 82866 or polypeptide SEQ ID NO. 82867, respectively, or a homolog or a fragment thereof, is increased or generated, or if the activity phosphoenolpyruvate carboxykinase is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 79 to 87-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a B3427-protein, or if the activity of the polypeptide B3427, preferably represented by SEQ ID NO. 42580, or a homolog or fragment thereof, or if the activity of a polypeptide encoded by a nucleic acid molecule comprising the nucleic acid SEQ ID NO. 42579, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 42579 or polypeptide SEQ ID NO. 42580, respectively, or a homolog or a fragment thereof, is increased or generated, or if the activity B3427-protein is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 39 to 125-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a glycogen synthase, or if the activity of the polypeptide B3429, preferably represented by SEQ ID NO. 94052, or a homolog or fragment thereof, or if the activity of a polypeptide encoded by a nucleic acid molecule comprising the nucleic acid SEQ ID NO. 94051, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 94051 or polypeptide SEQ ID NO. 94052, respectively, or a homolog or a fragment thereof, is increased or generated, or if the activity glycogen synthase is increased or generated in a non-human organism, like a microorganism or a plant cell, plant or part thereof, especially with mitochondrial localization, whereby the respective line disclose in column 7 the fine chemical fructose. For example, an increase of the fructose of at least 1 percent, particularly in a range of 79 to 211-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a glycogen synthase, or if the activity of the polypeptide B3429, preferably represented by SEQ ID NO. 94052, or a homolog or fragment thereof, or if the activity of a polypeptide encoded by a nucleic acid molecule comprising the nucleic acid SEQ ID NO. 94051, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 94051 or polypeptide SEQ ID NO. 94052, respectively, or a homolog or a fragment thereof, is increased or generated, or if the activity glycogen synthase is increased or generated in a non-human organism, like a microorganism or a plant cell, plant or part thereof, especially with mitochondrial localization, whereby the respective line disclose in column 7 the fine chemical glucose. For example, an increase of the glucose of at least 1 percent, particularly in a range of 39 to 65-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a b3442-protein, or if the activity of the polypeptide B3442, preferably represented by SEQ ID NO. 94381, or a homolog or fragment thereof, or if the activity of a polypeptide encoded by a nucleic acid molecule comprising the nucleic acid SEQ ID NO. 94380, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 94380 or polypeptide SEQ ID NO. 94381, respectively, or a homolog or a fragment thereof, is increased or generated, or if the activity b3442-protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 27 to 82-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a B3443-protein, or if the activity of the polypeptide B3443, preferably represented by SEQ ID NO. 146441, or a homolog or fragment thereof, or if the activity of a polypeptide encoded by a nucleic acid molecule comprising the nucleic acid SEQ ID NO. 146440, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 146440 or polypeptide SEQ ID NO. 146441, respectively, or a homolog or a fragment thereof, is increased or generated, or if the activity B3443-protein is increased or generated in a non-human organism, like a microorganism or a plant cell, plant or part thereof, especially with plastidic localization, whereby the respective line disclose in column 7 the fine chemical 3,4,5-trihydroxypentanoic acid. For example, an increase of the 3,4,5-trihydroxypentanoic acid of at least 1 percent, partitularly in a range of 44 to 69-percent is conferred as compared to a corresponding non-transformed wild type non-human organism.

Accordingly, in one embodiment, an increase of saccharic acid in a non-human organism, as compared to a corresponding non-transformed wild type non-human organism, is conferred in the process of the invention, if the activity of a polypeptide showing the activity of a sn-glycerol-3-phosphate transport system permease protein, or if the activity of the polypeptide B3451, preferably represented by SEQ ID NO. 116173, or a homolog or fragment thereof, or if the activity of a polypeptide encoded by a nucleic acid molecule comprising the nucleic acid SEQ ID NO. 116172, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 116172 or polypeptide SEQ ID NO. 116173, respectively, or a homolog or a fragment thereof, is increased or generated, or if the activity sn-glycerol-3-phosphate transport system permease protein is increased or generated in a non-human organism, like a microorganism or a plant cell, plant or part thereof, especially with plastidic localization, whereby the respective line disclose in column 7 the fine chemical saccharic acid. For example, an increase of the saccharic acid of at least 1 percent, particularly in a range of 53 to 59-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a high-affinity branched-chain amino acid transport protein, or if the activity of the polypeptide B3457, preferably represented by SEQ ID NO. 146473, or a homolog or fragment thereof, or if the activity of a polypeptide encoded by a nucleic acid molecule comprising the nucleic acid SEQ ID NO. 146472, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 146472 or polypeptide SEQ ID NO. 146473, respectively, or a homolog or a fragment thereof, is increased or generated, or if the activity high-affinity branched-chain amino acid transport protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 28 to 51-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a high-affinity branched-chain amino acid transport protein, or if the activity of the polypeptide B3457, preferably represented by SEQ ID NO. 146473, or a homolog or fragment thereof, or if the activity of a polypeptide encoded by a nucleic acid molecule comprising the nucleic acid SEQ ID NO. 146472, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 146472 or polypeptide SEQ ID NO. 146473, respectively, or a homolog or a fragment thereof, is increased or generated, or if the activity high-affinity branched-chain amino acid transport protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 24 to 54-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a trehalase, or if the activity of the polypeptide B3519, preferably represented by SEQ ID NO. 146647, or a homolog or fragment thereof, or if the activity of a polypeptide encoded by a nucleic acid molecule comprising the nucleic acid SEQ ID NO. 146646, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 146646 or polypeptide SEQ ID NO. 146647, respectively, or a homolog or a fragment thereof, is increased or generated, or if the activity trehalase is increased or generated in a non-human organism, like a microorganism or a plant cell, plant or part thereof, especially with plastidic localization, whereby the respective line disclose in column 7 the fine chemical fructose. For example, an increase of the fructose of at least 1 percent, particularly in a range of 24 to 71-percent is conferred as compared to a corresponding non-transformed wild type non-human organism.

Accordingly, in one embodiment, an increase of galactinol in a non-human organism, as compared to a corresponding non-transformed wild type non-human organism, is conferred in the process of the invention, if the activity of a polypeptide showing the activity of a trehalase, or if the activity of the polypeptide B3519, preferably represented by SEQ ID NO. 146647, or a homolog or fragment thereof, or if the activity of a polypeptide encoded by a nucleic acid molecule comprising the nucleic acid SEQ ID NO. 146646, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 146646 or polypeptide SEQ ID NO. 146647, respectively, or a homolog or a fragment thereof, is increased or generated, or if the activity trehalase is increased or generated in a non-human organism, like a microorganism or a plant cell, plant or part thereof, especially with plastidic localization, whereby the respective line disclose in column 7 the fine chemical galactinol. For example, an increase of the galactinol of at least 1 percent, particularly in a range of 40 to 107-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a mannitol-1-phosphate dehydrogenase, or if the activity of the polypeptide B3600, preferably represented by SEQ ID NO. 146733, or a homolog or fragment thereof, or if the activity of a polypeptide encoded by a nucleic acid molecule comprising the nucleic acid SEQ ID NO. 146732, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 146732 or polypeptide SEQ ID NO. 146733, respectively, or a homolog or a fragment thereof, is increased or generated, or if the activity mannitol-1-phosphate dehydrogenase is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 69 to 185-percent is conferred as compared to a corresponding non-transformed wild type non-human organism.

Accordingly, in one embodiment, an increase of mannitol in a non-human organism, as compared to a corresponding non-transformed wild type non-human organism, is conferred in the process of the invention, if the activity of a polypeptide showing the activity of a mannitol-1-phosphate dehydrogenase, or if the activity of the polypeptide B3600, preferably represented by SEQ ID NO. 146733, or a homolog or fragment thereof, or if the activity of a polypeptide encoded by a nucleic acid molecule comprising the nucleic acid SEQ ID NO. 146732, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 146732 or polypeptide SEQ ID NO. 146733, respectively, or a homolog or a fragment thereof, is increased or generated, or if the activity mannitol-1-phosphate dehydrogenase is increased or generated in a non-human organism, like a microorganism or a plant cell, plant or part thereof, especially with non-targeted localization, whereby the respective line disclose in column 7 the fine chemical mannitol. For example, an increase of the mannitol of at least 1 percent, particularly in a range of 422 to 16165-percent is conferred as compared to a corresponding non-transformed wild type non-human organism.

Accordingly, in one embodiment, an increase of sorbitol in a non-human organism, as compared to a corresponding non-transformed wild type non-human organism, is conferred in the process of the invention, if the activity of a polypeptide showing the activity of a mannitol-1-phosphate dehydrogenase, or if the activity of the polypeptide B3600, preferably represented by SEQ ID NO. 146733, or a homolog or fragment thereof, or if the activity of a polypeptide encoded by a nucleic acid molecule comprising the nucleic acid SEQ ID NO. 146732, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 146732 or polypeptide SEQ ID NO. 146733, respectively, or a homolog or a fragment thereof, is increased or generated, or if the activity mannitol-1-phosphate dehydrogenase is increased or generated in a non-human organism, like a microorganism or a plant cell, plant or part thereof, especially with non-targeted localization, whereby the respective line disclose in column 7 the fine chemical sorbitol. For example, an increase of the sorbitol of at least 1 percent, particularly in a range of 3140 to 12520-percent is conferred as compared to a corresponding non-transformed wild type non-human organism.

Accordingly, in one embodiment, an increase of galactinol in a non-human organism, as compared to a corresponding non-transformed wild type non-human organism, is conferred in the process of the invention, if the activity of a polypeptide showing the activity of a ketol-acid reductoisomerase, or if the activity of the polypeptide B3774, preferably represented by SEQ ID NO. 94387, or a homolog or fragment thereof, or if the activity of a polypeptide encoded by a nucleic acid molecule comprising the nucleic acid SEQ ID NO. 94386, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 94386 or polypeptide SEQ ID NO. 94387, respectively, or a homolog or a fragment thereof, is increased or generated, or if the activity ketol-acid reductoisomerase is increased or generated in a non-human organism, like a microorganism or a plant cell, plant or part thereof, especially with non-targeted localization, whereby the respective line disclose in column 7 the fine chemical galactinol. For example, an increase of the galactinol of at least 1 percent, particularly in a range of 37 to 119-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a b3777-protein, or if the activity of the polypeptide B3777, preferably represented by SEQ ID NO. 136099, or a homolog or fragment thereof, or if the activity of a polypeptide encoded by a nucleic acid molecule comprising the nucleic acid SEQ ID NO. 136098, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 136098 or polypeptide SEQ ID NO. 136099, respectively, or a homolog or a fragment thereof, is increased or generated, or if the activity b3777-protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 26 to 50-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a b3817-protein, or if the activity of the polypeptide B3817, preferably represented by SEQ ID NO. 10709, or a homolog or fragment thereof, or if the activity of a polypeptide encoded by a nucleic acid molecule comprising the nucleic acid SEQ ID NO. 10708, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 10708 or polypeptide SEQ ID NO. 10709, respectively, or a homolog or a fragment thereof, is increased or generated, or if the activity b3817-protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 38 to 203-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a b3817-protein, or if the activity of the polypeptide B3817, preferably represented by SEQ ID NO. 10709, or a homolog or fragment thereof, or if the activity of a polypeptide encoded by a nucleic acid molecule comprising the nucleic acid SEQ ID NO. 10708, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 10708 or polypeptide SEQ ID NO. 10709, respectively, or a homolog or a fragment thereof, is increased or generated, or if the activity b3817-protein is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 220 to 467-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a b3818-protein, or if the activity of the polypeptide B3818, preferably represented by SEQ ID NO. 146835, or a homolog or fragment thereof, or if the activity of a polypeptide encoded by a nucleic acid molecule comprising the nucleic acid SEQ ID NO. 146834, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 146834 or polypeptide SEQ ID NO. 146835, respectively, or a homolog or a fragment thereof, is increased or generated, or if the activity b3818-protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 31 to 45-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a threonine efflux protein, or if the activity of the polypeptide B3823, preferably represented by SEQ ID NO. 10727, or a homolog or fragment thereof, or if the activity of a polypeptide encoded by a nucleic acid molecule comprising the nucleic acid SEQ ID NO. 10726, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 10726 or polypeptide SEQ ID NO. 10727, respectively, or a homolog or a fragment thereof, is increased or generated, or if the activity threonine efflux protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 29 to 113-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a glutamine synthetase, or if the activity of the polypeptide B3870, preferably represented by SEQ ID NO. 101328, or a homolog or fragment thereof, or if the activity of a polypeptide encoded by a nucleic acid molecule comprising the nucleic acid SEQ ID NO. 101327, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 101327 or polypeptide SEQ ID NO. 101328, respectively, or a homolog or a fragment thereof, is increased or generated, or if the activity glutamine synthetase is increased or generated in a non-human organism, like a microorganism or a plant cell, plant or part thereof, especially with plastidic localization, whereby the respective line disclose in column 7 the fine chemical myo-inositol. For example, an increase of the myo-inositol of at least 1 percent, particularly in a range of 26 to 69-percent is conferred as compared to a corresponding non-transformed wild type non-human organism.

Accordingly, in one embodiment, an increase of verbascose in a non-human organism, as compared to a corresponding non-transformed wild type non-human organism, is conferred in the process of the invention, if the activity of a polypeptide showing the activity of a glutamine synthetase, or if the activity of the polypeptide B3870, preferably represented by SEQ ID NO. 101328, or a homolog or fragment thereof, or if the activity of a polypeptide encoded by a nucleic acid molecule comprising the nucleic acid SEQ ID NO. 101327, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 101327 or polypeptide SEQ ID NO. 101328, respectively, or a homolog or a fragment thereof, is increased or generated, or if the activity glutamine synthetase is increased or generated in a non-human organism, like a microorganism or a plant cell, plant or part thereof, especially with plastidic localization, whereby the respective line disclose in column 7 the fine chemical verbascose. For example, an increase of the verbascose of at least 1 percent, particularly in a range of 67 to 181-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a transcriptional regulator, or if the activity of the polypeptide B3872, preferably represented by SEQ ID NO. 83822, or a homolog or fragment thereof, or if the activity of a polypeptide encoded by a nucleic acid molecule comprising the nucleic acid SEQ ID NO. 83821, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 83821 or polypeptide SEQ ID NO. 83822, respectively, or a homolog or a fragment thereof, is increased or generated, or if the activity transcriptional regulator is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 27 to 59-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a b3989-protein, or if the activity of the polypeptide B3989, preferably represented by SEQ ID NO. 44373, or a homolog or fragment thereof, or if the activity of a polypeptide encoded by a nucleic acid molecule comprising the nucleic acid SEQ ID NO. 44372, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 44372 or polypeptide SEQ ID NO. 44373, respectively, or a homolog or a fragment thereof, is increased or generated, or if the activity b3989-protein is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 105 to 1389-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a b3989-protein, or if the activity of the polypeptide B3989, preferably represented by SEQ ID NO. 44373, or a homolog or fragment thereof, or if the activity of a polypeptide encoded by a nucleic acid molecule comprising the nucleic acid SEQ ID NO. 44372, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 44372 or polypeptide SEQ ID NO. 44373, respectively, or a homolog or a fragment thereof, is increased or generated, or if the activity b3989-protein is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 90 to 370-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a b3989-protein, or if the activity of the polypeptide B3989, preferably represented by SEQ ID NO. 44373, or a homolog or fragment thereof, or if the activity of a polypeptide encoded by a nucleic acid molecule comprising the nucleic acid SEQ ID NO. 44372, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 44372 or polypeptide SEQ ID NO. 44373, respectively, or a homolog or a fragment thereof, is increased or generated, or if the activity b3989-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 18 to 43-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a b3989-protein, or if the activity of the polypeptide B3989, preferably represented by SEQ ID NO. 44373, or a homolog or fragment thereof, or if the activity of a polypeptide encoded by a nucleic acid molecule comprising the nucleic acid SEQ ID NO. 44372, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 44372 or polypeptide SEQ ID NO. 44373, respectively, or a homolog or a fragment thereof, is increased or generated, or if the activity b3989-protein is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 95 to 272-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a b3989-protein, or if the activity of the polypeptide B3989, preferably represented by SEQ ID NO. 44373, or a homolog or fragment thereof, or if the activity of a polypeptide encoded by a nucleic acid molecule comprising the nucleic acid SEQ ID NO. 44372, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 44372 or polypeptide SEQ ID NO. 44373, respectively, or a homolog or a fragment thereof, is increased or generated, or if the activity b3989-protein is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 37 to 65-percent is conferred as compared to a corresponding non-transformed wild type non-human organism.

Accordingly, in one embodiment, an increase of galactinol in a non-human organism, as compared to a corresponding non-transformed wild type non-human organism, is conferred in the process of the invention, if the activity of a polypeptide showing the activity of a acetyltransferase, or if the activity of the polypeptide B4012, preferably represented by SEQ ID NO. 44379, or a homolog or fragment thereof, or if the activity of a polypeptide encoded by a nucleic acid molecule comprising the nucleic acid SEQ ID NO. 44378, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 44378 or polypeptide SEQ ID NO. 44379, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line disclose in column 7 the fine chemical galactinol. For example, an increase of the galactinol of at least 1 percent, particularly in a range of 37 to 102-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a b4029-protein, or if the activity of the polypeptide B4029, preferably represented by SEQ ID NO. 10741, or a homolog or fragment thereof, or if the activity of a polypeptide encoded by a nucleic acid molecule comprising the nucleic acid SEQ ID NO. 10740, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 10740 or polypeptide SEQ ID NO. 10741, respectively, or a homolog or a fragment thereof, is increased or generated, or if the activity b4029-protein is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 83 to 2120-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a b4029-protein, or if the activity of the polypeptide B4029, preferably represented by SEQ ID NO. 10741, or a homolog or fragment thereof, or if the activity of a polypeptide encoded by a nucleic acid molecule comprising the nucleic acid SEQ ID NO. 10740, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 10740 or polypeptide SEQ ID NO. 10741, respectively, or a homolog or a fragment thereof, is increased or generated, or if the activity b4029-protein is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 72 to 1072-percent is conferred as compared to a corresponding non-transformed wild type non-human organism.

Accordingly, in one embodiment, an increase of sedoheptulose-7-phosphate in a non-human organism, as compared to a corresponding non-transformed wild type non-human organism, is conferred in the process of the invention, if the activity of a polypeptide showing the activity of a b4029-protein, or if the activity of the polypeptide B4029, preferably represented by SEQ ID NO. 10741, or a homolog or fragment thereof, or if the activity of a polypeptide encoded by a nucleic acid molecule comprising the nucleic acid SEQ ID NO. 10740, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 10740 or polypeptide SEQ ID NO. 10741, respectively, or a homolog or a fragment thereof, is increased or generated, or if the activity b4029-protein is increased or generated in a non-human organism, like a microorganism or a plant cell, plant or part thereof, especially with non-targeted localization, whereby the respective line disclose in column 7 the fine chemical sedoheptulose-7-phosphate. For example, an increase of the sedoheptulose-7-phosphate of at least 1 percent, particularly in a range of 46 to 404-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a chorismate lyase, or if the activity of the polypeptide B4039, preferably represented by SEQ ID NO. 146849, or a homolog or fragment thereof, or if the activity of a polypeptide encoded by a nucleic acid molecule comprising the nucleic acid SEQ ID NO. 146848, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 146848 or polypeptide SEQ ID NO. 146849, respectively, or a homolog or a fragment thereof, is increased or generated, or if the activity chorismate lyase is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 20 to 54-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a chorismate lyase, or if the activity of the polypeptide B4039, preferably represented by SEQ ID NO. 146849, or a homolog or fragment thereof, or if the activity of a polypeptide encoded by a nucleic acid molecule comprising the nucleic acid SEQ ID NO. 146848, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 146848 or polypeptide SEQ ID NO. 146849, respectively, or a homolog or a fragment thereof, is increased or generated, or if the activity chorismate lyase is increased or generated in a non-human organism, like a microorganism or a plant cell, plant or part thereof, especially with plastidic localization, whereby the respective line disclose in column 7 the fine chemical 3,4,5-trihydroxypentanoic acid. For example, an increase of the 3,4,5-trihydroxypentanoic acid of at least 1 percent, particularly in a range of 33 to 87-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a b4050-protein, or if the activity of the polypeptide B4050, preferably represented by SEQ ID NO. 106533, or a homolog or fragment thereof, or if the activity of a polypeptide encoded by a nucleic acid molecule comprising the nucleic acid SEQ ID NO. 106532, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 106532 or polypeptide SEQ ID NO. 106533, respectively, or a homolog or a fragment thereof, is increased or generated, or if the activity b4050-protein is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 114 to 1402-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a b4050-protein, or if the activity of the polypeptide B4050, preferably represented by SEQ ID NO. 106533, or a homolog or fragment thereof, or if the activity of a polypeptide encoded by a nucleic acid molecule comprising the nucleic acid SEQ ID NO. 106532, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 106532 or polypeptide SEQ ID NO. 106533, respectively, or a homolog or a fragment thereof, is increased or generated, or if the activity b4050-protein is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 102 to 725-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a b4050-protein, or if the activity of the polypeptide B4050, preferably represented by SEQ ID NO. 106533, or a homolog or fragment thereof, or if the activity of a polypeptide encoded by a nucleic acid molecule comprising the nucleic acid SEQ ID NO. 106532, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 106532 or polypeptide SEQ ID NO. 106533, respectively, or a homolog or a fragment thereof, is increased or generated, or if the activity b4050-protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 27 to 309-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a b4056-protein, or if the activity of the polypeptide B4056, preferably represented by SEQ ID NO. 94543, or a homolog or fragment thereof, or if the activity of a polypeptide encoded by a nucleic acid molecule comprising the nucleic acid SEQ ID NO. 94542, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 94542 or polypeptide SEQ ID NO. 94543, respectively, or a homolog or a fragment thereof, is increased or generated, or if the activity b4056-protein is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 140 to 163-percent is conferred as compared to a corresponding non-transformed wild type non-human organism.

Accordingly, in one embodiment, an increase of galactinol in a non-human organism, as compared to a corresponding non-transformed wild type non-human organism, is conferred in the process of the invention, if the activity of a polypeptide showing the activity of a acetylcoenzyme A synthetase, or if the activity of the polypeptide B4069, preferably represented by SEQ ID NO. 126422, or a homolog or fragment thereof, or if the activity of a polypeptide encoded by a nucleic acid molecule comprising the nucleic acid SEQ ID NO. 126421, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 126421 or polypeptide SEQ ID NO. 126422, respectively, or a homolog or a fragment thereof, is increased or generated, or if the activity acetyl-coenzyme A synthetase is increased or generated in a non-human organism, like a microorganism or a plant cell, plant or part thereof, especially with plastidic localization, whereby the respective line disclose in column 7 the fine chemical galactinol. For example, an increase of the galactinol of at least 1 percent, particularly in a range of 47 to 118-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a lipoprotein precursor, or if the activity of the polypeptide B4189, preferably represented by SEQ ID NO. 146922, or a homolog or fragment thereof, or if the activity of a polypeptide encoded by a nucleic acid molecule comprising the nucleic acid SEQ ID NO. 146921, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 146921 or polypeptide SEQ ID NO. 146922, respectively, or a homolog or a fragment thereof, is increased or generated, or if the activity lipoprotein precursor is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 31 to 67-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a lipoprotein precursor, or if the activity of the polypeptide B4189, preferably represented by SEQ ID NO. 146922, or a homolog or fragment thereof, or if the activity of a polypeptide encoded by a nucleic acid molecule comprising the nucleic acid SEQ ID NO. 146921, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 146921 or polypeptide SEQ ID NO. 146922, respectively, or a homolog or a fragment thereof, is increased or generated, or if the activity lipoprotein precursor is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 43 to 74-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a b4250-protein, or if the activity of the polypeptide B4250, preferably represented by SEQ ID NO. 94736, or a homolog or fragment thereof, or if the activity of a polypeptide encoded by a nucleic acid molecule comprising the nucleic acid SEQ ID NO. 94735, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 94735 or polypeptide SEQ ID NO. 94736, respectively, or a homolog or a fragment thereof, is increased or generated, or if the activity b4250-protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 31 to 169-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a b4250-protein, or if the activity of the polypeptide B4250, preferably represented by SEQ ID NO. 94736, or a homolog or fragment thereof, or if the activity of a polypeptide encoded by a nucleic acid molecule comprising the nucleic acid SEQ ID NO. 94735, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 94735 or polypeptide SEQ ID NO. 94736, respectively, or a homolog or a fragment thereof, is increased or generated, or if the activity b4250-protein is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 188 to 278-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a acetyltransferase, or if the activity of the polypeptide B4256, preferably represented by SEQ ID NO. 45322, or a homolog or fragment thereof, or if the activity of a polypeptide encoded by a nucleic acid molecule comprising the nucleic acid SEQ ID NO. 45321, preferably the coding region thereof, or a homolog or fragment thereof, e.g. derived from Escherichia coli, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 45321 or polypeptide SEQ ID NO. 45322, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 219 to 2357-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a acetyltransferase, or if the activity of the polypeptide B4256, preferably represented by SEQ ID NO. 45322, or a homolog or fragment thereof, or if the activity of a polypeptide encoded by a nucleic acid molecule comprising the nucleic acid SEQ ID NO. 45321, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 45321 or polypeptide SEQ ID NO. 45322, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 88 to 504-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a gluconate transport system permease 3, or if the activity of the polypeptide B4321, preferably represented by SEQ ID NO. 45395, or a homolog or fragment thereof, or if the activity of a polypeptide encoded by a nucleic acid molecule comprising the nucleic acid SEQ ID NO. 45394, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 45394 or polypeptide SEQ ID NO. 45395, respectively, or a homolog or a fragment thereof, is increased or generated, or if the activity gluconate transport system permease 3 is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 104 to 177-percent is conferred as compared to a corresponding non-transformed wild type non-human organism.

Accordingly, in one embodiment, an increase of galactinol in a non-human organism, as compared to a corresponding non-transformed wild type non-human organism, is conferred in the process of the invention, if the activity of a polypeptide showing the activity of a purinenucleoside phosphorylase, or if the activity of the polypeptide B4384, preferably represented by SEQ ID NO. 45557, or a homolog or fragment thereof, or if the activity of a polypeptide encoded by a nucleic acid molecule comprising the nucleic acid SEQ ID NO. 45556, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 45556 or polypeptide SEQ ID NO. 45557, respectively, or a homolog or a fragment thereof, is increased or generated, or if the activity purine-nucleoside phosphorylase is increased or generated in a non-human organism, like a microorganism or a plant cell, plant or part thereof, especially with non-targeted localization, whereby the respective line disclose in column 7 the fine chemical galactinol. For example, an increase of the galactinol of at least 1 percent, particularly in a range of 56 to 127-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a lysine decarboxylase, or if the activity of the polypeptide D90900, preferably represented by SEQ ID NO. 84080, or a homolog or fragment thereof, or if the activity of a polypeptide encoded by a nucleic acid molecule comprising the nucleic acid SEQ ID NO. 84079, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 84079 or polypeptide SEQ ID NO. 84080, respectively, or a homolog or a fragment thereof, is increased or generated, or if the activity lysine decarboxylase is increased or generated in a non-human organism, like a microorganism or a plant cell, plant or part thereof, especially with plastidic localization, whereby the respective line disclose in column 7 the fine chemical myo-inositol. For example, an increase of the myo-inositol of at least 1 percent, particularly in a range of 37 to 67-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a glutaredoxin, or if the activity of the polypeptide GM02LC12622, preferably represented by SEQ ID NO. 10812, or a homolog or fragment thereof, or if the activity of a polypeptide encoded by a nucleic acid molecule comprising the nucleic acid SEQ ID NO. 10811, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 10811 or polypeptide SEQ ID NO. 10812, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 222 to 4601-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a glutaredoxin, or if the activity of the polypeptide GM02LC12622, preferably represented by SEQ ID NO. 10812, or a homolog or fragment thereof, or if the activity of a polypeptide encoded by a nucleic acid molecule comprising the nucleic acid SEQ ID NO. 10811, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 10811 or polypeptide SEQ ID NO. 10812, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 88 to 366-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a glutaredoxin, or if the activity of the polypeptide GM02LC12622, preferably represented by SEQ ID NO. 10812, or a homolog or fragment thereof, or if the activity of a polypeptide encoded by a nucleic acid molecule comprising the nucleic acid SEQ ID NO. 10811, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 10811 or polypeptide SEQ ID NO. 10812, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 26 to 99-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a glutaredoxin, or if the activity of the polypeptide GM02LC12622, preferably represented by SEQ ID NO. 10812, or a homolog or fragment thereof, or if the activity of a polypeptide encoded by a nucleic acid molecule comprising the nucleic acid SEQ ID NO. 10811, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 10811 or polypeptide SEQ ID NO. 10812, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 25 to 119-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a glutaredoxin, or if the activity of the polypeptide GM02LC12622, preferably represented by SEQ ID NO. 10812, or a homolog or fragment thereof, or if the activity of a polypeptide encoded by a nucleic acid molecule comprising the nucleic acid SEQ ID NO. 10811, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 10811 or polypeptide SEQ ID NO. 10812, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 30 to 134-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a transcription factor, or if the activity of the polypeptide GM02LC19289, preferably represented by SEQ ID NO. 46752, or a homolog or fragment thereof, or if the activity of a polypeptide encoded by a nucleic acid molecule comprising the nucleic acid SEQ ID NO. 46751, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 46751 or polypeptide SEQ ID NO. 46752, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 35 to 110-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a transcription factor, or if the activity of the polypeptide GM02LC19289, preferably represented by SEQ ID NO. 46752, or a homolog or fragment thereof, or if the activity of a polypeptide encoded by a nucleic acid molecule comprising the nucleic acid SEQ ID NO. 46751, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 46751 or polypeptide SEQ ID NO. 46752, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 39 to 119-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a transcription factor, or if the activity of the polypeptide GM02LC21368, preferably represented by SEQ ID NO. 84199, or a homolog or fragment thereof, or if the activity of a polypeptide encoded by a nucleic acid molecule comprising the nucleic acid SEQ ID NO. 84198, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 84198 or polypeptide SEQ ID NO. 84199, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 82 to 665-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a GM02LC38418-protein, or if the activity of the polypeptide GM02LC38418, preferably represented by SEQ ID NO. 116313, or a homolog or fragment thereof, or if the activity of a polypeptide encoded by a nucleic acid molecule comprising the nucleic acid SEQ ID NO. 116312, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 116312 or polypeptide SEQ ID NO. 116313, respectively, or a homolog or a fragment thereof, is increased or generated, or if the activity GM02LC38418-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 46 to 57-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a cyclin D, or if the activity of the polypeptide GM02LC44512, preferably represented by SEQ ID NO. 46851, or a homolog or fragment thereof, or if the activity of a polypeptide encoded by a nucleic acid molecule comprising the nucleic acid SEQ ID NO. 46850, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 46850 or polypeptide SEQ ID NO. 46851, respectively, or a homolog or a fragment thereof, is increased or generated, or if the activity cyclin D is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 47 to 85-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a cyclin D, or if the activity of the polypeptide GM02LC44512, preferably represented by SEQ ID NO. 46851, or a homolog or fragment thereof, or if the activity of a polypeptide encoded by a nucleic acid molecule comprising the nucleic acid SEQ ID NO. 46850, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 46850 or polypeptide SEQ ID NO. 46851, respectively, or a homolog or a fragment thereof, is increased or generated, or if the activity cyclin D is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 60 to 997-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a cyclin D, or if the activity of the polypeptide GM02LC44512, preferably represented by SEQ ID NO. 46851, or a homolog or fragment thereof, or if the activity of a polypeptide encoded by a nucleic acid molecule comprising the nucleic acid SEQ ID NO. 46850, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 46850 or polypeptide SEQ ID NO. 46851, respectively, or a homolog or a fragment thereof, is increased or generated, or if the activity cyclin D is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 28 to 94-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a GM02LC46-protein, or if the activity of the polypeptide GM02LC46, preferably represented by SEQ ID NO. 47027, or a homolog or fragment thereof, or if the activity of a polypeptide encoded by a nucleic acid molecule comprising the nucleic acid SEQ ID NO. 47026, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 47026 or polypeptide SEQ ID NO. 47027, respectively, or a homolog or a fragment thereof, is increased or generated, or if the activity GM02LC46-protein is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 35 to 69-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a transcription factor, or if the activity of the polypeptide GM02LC6021, preferably represented by SEQ ID NO. 47106, or a homolog or fragment thereof, or if the activity of a polypeptide encoded by a nucleic acid molecule comprising the nucleic acid SEQ ID NO. 47105, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 47105 or polypeptide SEQ ID NO. 47106, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 38 to 92-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a alkaline phosphatase, or if the activity of the polypeptide Sll0222, preferably represented by SEQ ID NO. 146940, or a homolog or fragment thereof, or if the activity of a polypeptide encoded by a nucleic acid molecule comprising the nucleic acid SEQ ID NO. 146939, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 146939 or polypeptide SEQ ID NO. 146940, respectively, or a homolog or a fragment thereof, is increased or generated, or if the activity alkaline phosphatase is increased or generated in a non-human organism, like a microorganism or a plant cell, plant or part thereof, especially with plastidic localization, whereby the respective line disclose in column 7 the fine chemical myo-inositol. For example, an increase of the myo-inositol of at least 1 percent, particularly in a range of 26 to 48-percent is conferred as compared to a corresponding non-transformed wild type non-human organism.

Accordingly, in one embodiment, an increase of verbascose in a non-human organism, as compared to a corresponding non-transformed wild type non-human organism, is conferred in the process of the invention, if the activity of a polypeptide showing the activity of a flavodoxin, or if the activity of the polypeptide Sll0248, preferably represented by SEQ ID NO. 49144, or a homolog or fragment thereof, or if the activity of a polypeptide encoded by a nucleic acid molecule comprising the nucleic acid SEQ ID NO. 49143, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 49143 or polypeptide SEQ ID NO. 49144, respectively, or a homolog or a fragment thereof, is increased or generated, or if the activity flavodoxin is increased or generated in a non-human organism, like a microorganism or a plant cell, plant or part thereof, especially with plastidic localization, whereby the respective line disclose in column 7 the fine chemical verbascose. For example, an increase of the verbascose of at least 1 percent, particularly in a range of 88 to 378-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a sll0254-protein, or if the activity of the polypeptide Sll0254, preferably represented by SEQ ID NO. 49801, or a homolog or fragment thereof, or if the activity of a polypeptide encoded by a nucleic acid molecule comprising the nucleic acid SEQ ID NO. 49800, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 49800 or polypeptide SEQ ID NO. 49801, respectively, or a homolog or a fragment thereof, is increased or generated, or if the activity sll0254-protein is increased or generated in a non-human organism, like a microorganism or a plant cell, plant or part thereof, especially with mitochondrial localization, whereby the respective line disclose in column 7 the fine chemical sucrose. For example, an increase of the sucrose of at least 1 percent, particularly in a range of 25 to 62-percent is conferred as compared to a corresponding non-transformed wild type non-human organism.

Accordingly, in one embodiment, an increase of verbascose in a non-human organism, as compared to a corresponding non-transformed wild type non-human organism, is conferred in the process of the invention, if the activity of a polypeptide showing the activity of a sll0281-protein, or if the activity of the polypeptide Sll0281, preferably represented by SEQ ID NO. 84866, or a homolog or fragment thereof, or if the activity of a polypeptide encoded by a nucleic acid molecule comprising the nucleic acid SEQ ID NO. 84865, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 84865 or polypeptide SEQ ID NO. 84866, respectively, or a homolog or a fragment thereof, is increased or generated, or if the activity sll0281-protein is increased or generated in a non-human organism, like a microorganism or a plant cell, plant or part thereof, especially with plastidic localization, whereby the respective line disclose in column 7 the fine chemical verbascose. For example, an increase of the verbascose of at least 1 percent, particularly in a range of 107 to 525-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a sll0354-protein, or if the activity of the polypeptide Sll0354, preferably represented by SEQ ID NO. 50071, or a homolog or fragment thereof, or if the activity of a polypeptide encoded by a nucleic acid molecule comprising the nucleic acid SEQ ID NO. 50070, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 50070 or polypeptide SEQ ID NO. 50071, respectively, or a homolog or a fragment thereof, is increased or generated, or if the activity sll0354-protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 25 to 38-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a bifunctional protein (phosphoribosyltransferase and regulatory protein), or if the activity of the polypeptide Sll0368, preferably represented by SEQ ID NO. 106921, or a homolog or fragment thereof, or if the activity of a polypeptide encoded by a nucleic acid molecule comprising the nucleic acid SEQ ID NO. 106920, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 106920 or polypeptide SEQ ID NO. 106921, respectively, or a homolog or a fragment thereof, is increased or generated, or if the activity bifunctional protein (phosphoribosyltransferase and regulatory protein) is increased or generated in a non-human organism, like a microorganism or a plant cell, plant or part thereof, especially with mitochondrial localization, whereby the respective line disclose in column 7 the fine chemical myo-inositol. For example, an increase of the myo-inositol of at least 1 percent, particularly in a range of 25 to 77-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a sll0418-protein, or if the activity of the polypeptide Sll0418, preferably represented by SEQ ID NO. 94943, or a homolog or fragment thereof, or if the activity of a polypeptide encoded by a nucleic acid molecule comprising the nucleic acid SEQ ID NO. 94942, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 94942 or polypeptide SEQ ID NO. 94943, respectively, or a homolog or a fragment thereof, is increased or generated, or if the activity sll0418-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 43 to 164-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a sll0418-protein, or if the activity of the polypeptide Sll0418, preferably represented by SEQ ID NO. 94943, or a homolog or fragment thereof, or if the activity of a polypeptide encoded by a nucleic acid molecule comprising the nucleic acid SEQ ID NO. 94942, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 94942 or polypeptide SEQ ID NO. 94943, respectively, or a homolog or a fragment thereof, is increased or generated, or if the activity sll0418-protein is increased or generated in a non-human organism, like a microorganism or a plant cell, plant or part thereof, especially with mitochondrial localization, whereby the respective line disclose in column 7 the fine chemical myo-inositol. For example, an increase of the myo-inositol of at least 1 percent, particularly in a range of 33 to 56-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a sll0418-protein, or if the activity of the polypeptide Sll0418, preferably represented by SEQ ID NO. 94943, or a homolog or fragment thereof, or if the activity of a polypeptide encoded by a nucleic acid molecule comprising the nucleic acid SEQ ID NO. 94942, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 94942 or polypeptide SEQ ID NO. 94943, respectively, or a homolog or a fragment thereof, is increased or generated, or if the activity sll0418-protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 26 to 38-percent is conferred as compared to a corresponding non-transformed wild type non-human organism.

Accordingly, in one embodiment, an increase of saccharic acid in a non-human organism, as compared to a corresponding non-transformed wild type non-human organism, is conferred in the process of the invention, if the activity of a polypeptide showing the activity of a sll0418-protein, or if the activity of the polypeptide Sll0418, preferably represented by SEQ ID NO. 94943, or a homolog or fragment thereof, or if the activity of a polypeptide encoded by a nucleic acid molecule comprising the nucleic acid SEQ ID NO. 94942, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 94942 or polypeptide SEQ ID NO. 94943, respectively, or a homolog or a fragment thereof, is increased or generated, or if the activity sll0418-protein is increased or generated in a non-human organism, like a microorganism or a plant cell, plant or part thereof, especially with non-targeted localization, whereby the respective line disclose in column 7 the fine chemical saccharic acid. For example, an increase of the saccharic acid of at least 1 percent, particularly in a range of 51 to 100-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a sll0418-protein, or if the activity of the polypeptide Sll0418, preferably represented by SEQ ID NO. 94943, or a homolog or fragment thereof, or if the activity of a polypeptide encoded by a nucleic acid molecule comprising the nucleic acid SEQ ID NO. 94942, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 94942 or polypeptide SEQ ID NO. 94943, respectively, or a homolog or a fragment thereof, is increased or generated, or if the activity sll0418-protein is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 37 to 157-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a urease subunit, or if the activity of the polypeptide Sll0420, preferably represented by SEQ ID NO. 50105, or a homolog or fragment thereof, or if the activity of a polypeptide encoded by a nucleic acid molecule comprising the nucleic acid SEQ ID NO. 50104, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 50104 or polypeptide SEQ ID NO. 50105, respectively, or a homolog or a fragment thereof, is increased or generated, or if the activity urease subunit is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 23 to 36-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a glucokinase, or if the activity of the polypeptide Sll0593, preferably represented by SEQ ID NO. 85511, or a homolog or fragment thereof, or if the activity of a polypeptide encoded by a nucleic acid molecule comprising the nucleic acid SEQ ID NO. 85510, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 85510 or polypeptide SEQ ID NO. 85511, respectively, or a homolog or a fragment thereof, is increased or generated, or if the activity glucokinase is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 35 to 72-percent is conferred as compared to a corresponding non-transformed wild type non-human organism.

Accordingly, in one embodiment, an increase of saccharic acid in a non-human organism, as compared to a corresponding non-transformed wild type non-human organism, is conferred in the process of the invention, if the activity of a polypeptide showing the activity of a urease accessory protein, or if the activity of the polypeptide Sll0643, preferably represented by SEQ ID NO. 146946, or a homolog or fragment thereof, or if the activity of a polypeptide encoded by a nucleic acid molecule comprising the nucleic acid SEQ ID NO. 146945, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 146945 or polypeptide SEQ ID NO. 146946, respectively, or a homolog or a fragment thereof, is increased or generated, or if the activity urease accessory protein is increased or generated in a non-human organism, like a microorganism or a plant cell, plant or part thereof, especially with non-targeted localization, whereby the respective line disclose in column 7 the fine chemical saccharic acid. For example, an increase of the saccharic acid of at least 1 percent, particularly in a range of 46 to 111-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a binding-protein-dependent transport systems inner membrane component, or if the activity of the polypeptide Sll0833, preferably represented by SEQ ID NO. 101708, or a homolog or fragment thereof, or if the activity of a polypeptide encoded by a nucleic acid molecule comprising the nucleic acid SEQ ID NO. 101707, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 101707 or polypeptide SEQ ID NO. 101708, respectively, or a homolog or a fragment thereof, is increased or generated, or if the activity binding-protein-dependent transport systems inner membrane component is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 33 to 132-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a binding-protein-dependent transport systems inner membrane component, or if the activity of the polypeptide Sll0833, preferably represented by SEQ ID NO. 101708, or a homolog or fragment thereof, or if the activity of a polypeptide encoded by a nucleic acid molecule comprising the nucleic acid SEQ ID NO. 101707, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 101707 or polypeptide SEQ ID NO. 101708, respectively, or a homolog or a fragment thereof, is increased or generated, or if the activity binding-protein-dependent transport systems inner membrane component is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 39 to 122-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a 3-deoxy-7-phosphoheptulonate synthase, or if the activity of the polypeptide Sll0934, preferably represented by SEQ ID NO. 52247, or a homolog or fragment thereof, or if the activity of a polypeptide encoded by a nucleic acid molecule comprising the nucleic acid SEQ ID NO. 52246, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 52246 or polypeptide SEQ ID NO. 52247, respectively, or a homolog or a fragment thereof, is increased or generated, or if the activity 3-deoxy-7-phosphoheptulonate synthase is increased or generated in a non-human organism, like a microorganism or a plant cell, plant or part thereof, especially with mitochondrial localization, whereby the respective line disclose in column 7 the fine chemical glucose-1-phosphate. For example, an increase of the glucose-1-phosphate of at least 1 percent, particularly in a range of 39 to 58-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a glycogen synthase, or if the activity of the polypeptide Sll0945, preferably represented by SEQ ID NO. 52365, or a homolog or fragment thereof, or if the activity of a polypeptide encoded by a nucleic acid molecule comprising the nucleic acid SEQ ID NO. 52364, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 52364 or polypeptide SEQ ID NO. 52365, respectively, or a homolog or a fragment thereof, is increased or generated, or if the activity glycogen synthase is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 94 to 495-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a glycogen synthase, or if the activity of the polypeptide Sll0945, preferably represented by SEQ ID NO. 52365, or a homolog or fragment thereof, or if the activity of a polypeptide encoded by a nucleic acid molecule comprising the nucleic acid SEQ ID NO. 52364, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 52364 or polypeptide SEQ ID NO. 52365, respectively, or a homolog or a fragment thereof, is increased or generated, or if the activity glycogen synthase is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 30 to 82-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a coproporphyrinogen oxidase, or if the activity of the polypeptide Sll1185, preferably represented by SEQ ID NO. 53190, or a homolog or fragment thereof, or if the activity of a polypeptide encoded by a nucleic acid molecule comprising the nucleic acid SEQ ID NO. 53189, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 53189 or polypeptide SEQ ID NO. 53190, respectively, or a homolog or a fragment thereof, is increased or generated, or if the activity coproporphyrinogen oxidase is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 32 to 41-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a short chain dehydrogenase, or if the activity of the polypeptide SLL1308, preferably represented by SEQ ID NO. 147209, or a homolog or fragment thereof, or if the activity of a polypeptide encoded by a nucleic acid molecule comprising the nucleic acid SEQ ID NO. 147208, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 147208 or polypeptide SEQ ID NO. 147209, respectively, or a homolog or a fragment thereof, is increased or generated, or if the activity short chain dehydrogenase is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 34 to 69-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a glycogen (starch) synthase, or if the activity of the polypeptide Sll1393, preferably represented by SEQ ID NO. 53457, or a homolog or fragment thereof, or if the activity of a polypeptide encoded by a nucleic acid molecule comprising the nucleic acid SEQ ID NO. 53456, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 53456 or polypeptide SEQ ID NO. 53457, respectively, or a homolog or a fragment thereof, is increased or generated, or if the activity glycogen (starch) synthase is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 71 to 157-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a carbamoylphosphate synthase subunit, or if the activity of the polypeptide Sll1498, preferably represented by SEQ ID NO. 95048, or a homolog or fragment thereof, or if the activity of a polypeptide encoded by a nucleic acid molecule comprising the nucleic acid SEQ ID NO. 95047, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 95047 or polypeptide SEQ ID NO. 95048, respectively, or a homolog or a fragment thereof, is increased or generated, or if the activity carbamoyl-phosphate synthase subunit is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 24 to 28-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a carbamoyl-phosphate synthase subunit, or if the activity of the polypeptide Sll1498, preferably represented by SEQ ID NO. 95048, or a homolog or fragment thereof, or if the activity of a polypeptide encoded by a nucleic acid molecule comprising the nucleic acid SEQ ID NO. 95047, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 95047 or polypeptide SEQ ID NO. 95048, respectively, or a homolog or a fragment thereof, is increased or generated, or if the activity carbamoyl-phosphate synthase subunit is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 38 to 68-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a 4-alpha-glucanotransferase, or if the activity of the polypeptide Sll1676, preferably represented by SEQ ID NO. 54898, or a homolog or fragment thereof, or if the activity of a polypeptide encoded by a nucleic acid molecule comprising the nucleic acid SEQ ID NO. 54897, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 54897 or polypeptide SEQ ID NO. 54898, respectively, or a homolog or a fragment thereof, is increased or generated, or if the activity 4-alpha-glucanotransferase is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 20 to 24-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a alanine dehydrogenase, or if the activity of the polypeptide Sll1682, preferably represented by SEQ ID NO. 55064, or a homolog or fragment thereof, or if the activity of a polypeptide encoded by a nucleic acid molecule comprising the nucleic acid SEQ ID NO. 55063, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 55063 or polypeptide SEQ ID NO. 55064, respectively, or a homolog or a fragment thereof, is increased or generated, or if the activity alanine dehydrogenase is increased or generated in a non-human organism, like a microorganism or a plant cell, plant or part thereof, especially with plastidic localization, whereby the respective line disclose in column 7 the fine chemical 3,4,5-trihydroxypentanoic acid. For example, an increase of the 3,4,5-trihydroxypentanoic acid of at least 1 percent, particularly in a range of 33 to 65-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a sll1761-protein, or if the activity of the polypeptide Sll1761, preferably represented by SEQ ID NO. 55380, or a homolog or fragment thereof, or if the activity of a polypeptide encoded by a nucleic acid molecule comprising the nucleic acid SEQ ID NO. 55379, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 55379 or polypeptide SEQ ID NO. 55380, respectively, or a homolog or a fragment thereof, is increased or generated, or if the activity sll1761-protein is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 62 to 157-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a acyl transferase, or if the activity of the polypeptide Sll1848, preferably represented by SEQ ID NO. 86448, or a homolog or fragment thereof, or if the activity of a polypeptide encoded by a nucleic acid molecule comprising the nucleic acid SEQ ID NO. 86447, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 86447 or polypeptide SEQ ID NO. 86448, respectively, or a homolog or a fragment thereof, is increased or generated, or if the activity acyl transferase is increased or generated in a non-human organism, like a microorganism or a plant cell, plant or part thereof, especially with plastidic localization, whereby the respective line disclose in column 7 the fine chemical glucose. For example, an increase of the glucose of at least 1-percent, particularly in a range of 115 to 341-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a cation-transporting ATPase, or if the activity of the polypeptide Sll1920, preferably represented by SEQ ID NO. 55386, or a homolog or fragment thereof, or if the activity of a polypeptide encoded by a nucleic acid molecule comprising the nucleic acid SEQ ID NO. 55385, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 55385 or polypeptide SEQ ID NO. 55386, respectively, or a homolog or a fragment thereof, is increased or generated, or if the activity cation-transporting ATPase is increased or generated in a non-human organism, like a microorganism or a plant cell, plant or part thereof, especially with mitochondrial localization, whereby the respective line disclose in column 7 the fine chemical glucose-1-phosphate. For example, an increase of the glucose-1-phosphate of at least 1 percent, particularly in a range of 20 to 106-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a cation-transporting ATPase, or if the activity of the polypeptide Sll1920, preferably represented by SEQ ID NO. 55386, or a homolog or fragment thereof, or if the activity of a polypeptide encoded by a nucleic acid molecule comprising the nucleic acid SEQ ID NO. 55385, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 55385 or polypeptide SEQ ID NO. 55386, respectively, or a homolog or a fragment thereof, is increased or generated, or if the activity cation-transporting ATPase is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 20 to 136-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a cation-transporting ATPase, or if the activity of the polypeptide Sll1920, preferably represented by SEQ ID NO. 55386, or a homolog or fragment thereof, or if the activity of a polypeptide encoded by a nucleic acid molecule comprising the nucleic acid SEQ ID NO. 55385, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 55385 or polypeptide SEQ ID NO. 55386, respectively, or a homolog or a fragment thereof, is increased or generated, or if the activity cation-transporting ATPase is increased or generated in a non-human organism, like a microorganism or a plant cell, plant or part thereof, especially with mitochondrial localization, whereby the respective line disclose in column 7 the fine chemical myo-inositol. For example, an increase of the myo-inositol of at least 1 percent, particularly in a range of 25 to 28-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a cation-transporting ATPase, or if the activity of the polypeptide Sll1920, preferably represented by SEQ ID NO. 55386, or a homolog or fragment thereof, or if the activity of a polypeptide encoded by a nucleic acid molecule comprising the nucleic acid SEQ ID NO. 55385, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 55385 or polypeptide SEQ ID NO. 55386, respectively, or a homolog or a fragment thereof, is increased or generated, or if the activity cation-transporting ATPase is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 85 to 216-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a glycosidase, or if the activity of the polypeptide Slr0237, preferably represented by SEQ ID NO. 55772, or a homolog or fragment thereof, or if the activity of a polypeptide encoded by a nucleic acid molecule comprising the nucleic acid SEQ ID NO. 55771, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 55771 or polypeptide SEQ ID NO. 55772, respectively, or a homolog or a fragment thereof, is increased or generated, or if the activity glycosidase is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 31 to 55-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a precorrin methylase, or if the activity of the polypeptide Slr0239, preferably represented by SEQ ID NO. 86541, or a homolog or fragment thereof, or if the activity of a polypeptide encoded by a nucleic acid molecule comprising the nucleic acid SEQ ID NO. 86540, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 86540 or polypeptide SEQ ID NO. 86541, respectively, or a homolog or a fragment thereof, is increased or generated, or if the activity precorrin methylase is increased or generated in a non-human organism, like a microorganism or a plant cell, plant or part thereof, especially with mitochondrial localization, whereby the respective line disclose in column 7 the fine chemical fructose. For example, an increase of the fructose of at least 1 percent, particularly in a range of 76 to 426-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a precorrin methylase, or if the activity of the polypeptide Slr0239, preferably represented by SEQ ID NO. 86541, or a homolog or fragment thereof, or if the activity of a polypeptide encoded by a nucleic acid molecule comprising the nucleic acid SEQ ID NO. 86540, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 86540 or polypeptide SEQ ID NO. 86541, respectively, or a homolog or a fragment thereof, is increased or generated, or if the activity precorrin methylase is increased or generated in a non-human organism, like a microorganism or a plant cell, plant or part thereof, especially with mitochondrial localization, whereby the respective line disclose in column 7 the fine chemical glucose. For example, an increase of the glucose of at least 1 percent, particularly in a range of 65 to 167-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a oxidoreductase, or if the activity of the polypeptide Slr0338, preferably represented by SEQ ID NO. 11991, or a homolog or fragment thereof, or if the activity of a polypeptide encoded by a nucleic acid molecule comprising the nucleic acid SEQ ID NO. 11990, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 11990 or polypeptide SEQ ID NO. 11991, respectively, or a homolog or a fragment thereof, is increased or generated, or if the activity oxidoreductase is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 18 to 64-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a oxidoreductase, or if the activity of the polypeptide Slr0338, preferably represented by SEQ ID NO. 11991, or a homolog or fragment thereof, or if the activity of a polypeptide encoded by a nucleic acid molecule comprising the nucleic acid SEQ ID NO. 11990, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 11990 or polypeptide SEQ ID NO. 11991, respectively, or a homolog or a fragment thereof, is increased or generated, or if the activity oxidoreductase is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 43 to 133-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a slr0600-protein, or if the activity of the polypeptide Slr0600, preferably represented by SEQ ID NO. 56515, or a homolog or fragment thereof, or if the activity of a polypeptide encoded by a nucleic acid molecule comprising the nucleic acid SEQ ID NO. 56514, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 56514 or polypeptide SEQ ID NO. 56515, respectively, or a homolog or a fragment thereof, is increased or generated, or if the activity slr0600-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 18 to 91-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a Glu/Leu/Phe/Val dehydrogenase , or if the activity of the polypeptide Slr0710, preferably represented by SEQ ID NO. 56895, or a homolog or fragment thereof, or if the activity of a polypeptide encoded by a nucleic acid molecule comprising the nucleic acid SEQ ID NO. 56894, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 56894 or polypeptide SEQ ID NO. 56895, respectively, or a homolog or a fragment thereof, is increased or generated, or if the activity Glu/Leu/Phe/Val dehydrogenase is increased or generated in a non-human organism, like a microorganism or a plant cell, plant or part thereof, especially with mitochondrial localization, whereby the respective line disclose in column 7 the fine chemical glucose-1-phosphate. For example, an increase of the glucose-1-phosphate of at least 1-percent, particularly in a range of 24 to 37-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a malate dehydrogenase, or if the activity of the polypeptide Slr0721, preferably represented by SEQ ID NO. 87398, or a homolog or fragment thereof, or if the activity of a polypeptide encoded by a nucleic acid molecule comprising the nucleic acid SEQ ID NO. 87397, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 87397 or polypeptide SEQ ID NO. 87398, respectively, or a homolog or a fragment thereof, is increased or generated, or if the activity malate dehydrogenase is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 24 to 119-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a geranylgeranyl pyrophosphate synthase, or if the activity of the polypeptide Slr0739, preferably represented by SEQ ID NO. 57236, or a homolog or fragment thereof, or if the activity of a polypeptide encoded by a nucleic acid molecule comprising the nucleic acid SEQ ID NO. 57235, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 57235 or polypeptide SEQ ID NO. 57236, respectively, or a homolog or a fragment thereof, is increased or generated, or if the activity geranylgeranyl pyrophosphate synthase is increased or generated in a non-human organism, like a microorganism or a plant cell, plant or part thereof, especially with plastidic localization, whereby the respective line disclose in column 7 the fine chemical sucrose. For example, an increase of the sucrose of at least 1 percent, particularly in a range of 26 to 34-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a circadian clock protein, or if the activity of the polypeptide Slr0757, preferably represented by SEQ ID NO. 147347, or a homolog or fragment thereof, or if the activity of a polypeptide encoded by a nucleic acid molecule comprising the nucleic acid SEQ ID NO. 147346, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 147346 or polypeptide SEQ ID NO. 147347, respectively, or a homolog or a fragment thereof, is increased or generated, or if the activity circadian clock protein is increased or generated in a non-human organism, like a microorganism or a plant cell, plant or part thereof, especially with mitochondrial localization, whereby the respective line disclose in column 7 the fine chemical fructose. For example, an increase of the fructose of at least 1 percent, particularly in a range of 100 to 243-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a circadian clock protein, or if the activity of the polypeptide Slr0757, preferably represented by SEQ ID NO. 147347, or a homolog or fragment thereof, or if the activity of a polypeptide encoded by a nucleic acid molecule comprising the nucleic acid SEQ ID NO. 147346, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 147346 or polypeptide SEQ ID NO. 147347, respectively, or a homolog or a fragment thereof, is increased or generated, or if the activity circadian clock protein is increased or generated in a non-human organism, like a microorganism or a plant cell, plant or part thereof, especially with mitochondrial localization, whereby the respective line disclose in column 7 the fine chemical glucose. For example, an increase of the glucose of at least 1 percent, particularly in a range of 72 to 114-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a circadian clock protein, or if the activity of the polypeptide Slr0757, preferably represented by SEQ ID NO. 147347, or a homolog or fragment thereof, or if the activity of a polypeptide encoded by a nucleic acid molecule comprising the nucleic acid SEQ ID NO. 147346, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 147346 or polypeptide SEQ ID NO. 147347, respectively, or a homolog or a fragment thereof, is increased or generated, or if the activity circadian clock protein is increased or generated in a non-human organism, like a microorganism or a plant cell, plant or part thereof, especially with mitochondrial localization, whereby the respective line disclose in column 7 the fine chemical glucose-1-phosphate. For example, an increase of the glucose-1-phosphate of at least 1 percent, particularly in a range of 39 to 56-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a amine oxidase, or if the activity of the polypeptide Slr0782, preferably represented by SEQ ID NO. 57680, or a homolog or fragment thereof, or if the activity of a polypeptide encoded by a nucleic acid molecule comprising the nucleic acid SEQ ID NO. 57679, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 57679 or polypeptide SEQ ID NO. 57680, respectively, or a homolog or a fragment thereof, is increased or generated, or if the activity amine oxidase is increased or generated in a non-human organism, like a microorganism or a plant cell, plant or part thereof, especially with mitochondrial localization, whereby the respective line disclose in column 7 the fine chemical glucose-1-phosphate. For example, an increase of the glucose-1-phosphate of at least 1 percent, particularly in a range of 33 to 68-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a kinase, or if the activity of the polypeptide Slr0862, preferably represented by SEQ ID NO. 87611, or a homolog or fragment thereof, or if the activity of a polypeptide encoded by a nucleic acid molecule comprising the nucleic acid SEQ ID NO. 87610, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 87610 or polypeptide SEQ ID NO. 87611, respectively, or a homolog or a fragment thereof, is increased or generated, or if the activity kinase is increased or generated in a non-human organism, like a microorganism or a plant cell, plant or part thereof, especially with mitochondrial localization, whereby the respective line disclose in column 7 the fine chemical fructose. For example, an increase of the fructose of at least 1 percent, particularly in a range of 80 to 98-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a kinase, or if the activity of the polypeptide Slr0862, preferably represented by SEQ ID NO. 87611, or a homolog or fragment thereof, or if the activity of a polypeptide encoded by a nucleic acid molecule comprising the nucleic acid SEQ ID NO. 87610, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 87610 or polypeptide SEQ ID NO. 87611, respectively, or a homolog or a fragment thereof, is increased or generated, or if the activity kinase is increased or generated in a non-human organism, like a microorganism or a plant cell, plant or part thereof, especially with mitochondrial localization, whereby the respective line disclose in column 7 the fine chemical glucose. For example, an increase of the glucose of at least 1 percent, particularly in a range of 54 to 88-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a Mg-protoporphyrin IX monomethyl ester oxidative cyclase 66 kD subunit, or if the activity of the polypeptide Slr0905, preferably represented by SEQ ID NO. 147408, or a homolog or fragment thereof, or if the activity of a polypeptide encoded by a nucleic acid molecule comprising the nucleic acid SEQ ID NO. 147407, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 147407 or polypeptide SEQ ID NO. 147408, respectively, or a homolog or a fragment thereof, is increased or generated, or if the activity Mg-protoporphyrin IX monomethyl ester oxidative cyclase 66 kD subunit is increased or generated in a non-human organism, like a microorganism or a plant cell, plant or part thereof, especially with mitochondrial localization, whereby the respective line disclose in column 7 the fine chemical glucose-1-phosphate. For example, an increase of the glucose-1-phosphate of at least 1 percent, particularly in a range of 32 to 57-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a permease protein of ABC transporter, or if the activity of the polypeptide Slr0949, preferably represented by SEQ ID NO. 119223, or a homolog or fragment thereof, or if the activity of a polypeptide encoded by a nucleic acid molecule comprising the nucleic acid SEQ ID NO. 119222, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 119222 or polypeptide SEQ ID NO. 119223, respectively, or a homolog or a fragment thereof, is increased or generated, or if the activity permease protein of ABC transporter is increased or generated in a non-human organism, like a microorganism or a plant cell, plant or part thereof, especially with mitochondrial localization, whereby the respective line disclose in column 7 the fine chemical myo-inositol. For example, an increase of the myo-inositol of at least 1 percent, particularly in a range of 25 to 56-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a dihydrolipoamide dehydrogenase, or if the activity of the polypeptide Slr1096, preferably represented by SEQ ID NO. 57735, or a homolog or fragment thereof, or if the activity of a polypeptide encoded by a nucleic acid molecule comprising the nucleic acid SEQ ID NO. 57734, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 57734 or polypeptide SEQ ID NO. 57735, respectively, or a homolog or a fragment thereof, is increased or generated, or if the activity dihydrolipoamide dehydrogenase is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1-percent, particularly in a range of 31 to 90-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a dihydrolipoamide dehydrogenase, or if the activity of the polypeptide Slr1096, preferably represented by SEQ ID NO. 57735, or a homolog or fragment thereof, or if the activity of a polypeptide encoded by a nucleic acid molecule comprising the nucleic acid SEQ ID NO. 57734, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 57734 or polypeptide SEQ ID NO. 57735, respectively, or a homolog or a fragment thereof, is increased or generated, or if the activity dihydrolipoamide dehydrogenase is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 81 to 216-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a slr1107-protein, or if the activity of the polypeptide Slr1107, preferably represented by SEQ ID NO. 101777, or a homolog or fragment thereof, or if the activity of a polypeptide encoded by a nucleic acid molecule comprising the nucleic acid SEQ ID NO. 101776, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 101776 or polypeptide SEQ ID NO. 101777, respectively, or a homolog or a fragment thereof, is increased or generated, or if the activity slr1107-protein is increased or generated in a non-human organism, like a microorganism or a plant cell, plant or part thereof, especially with plastidic localization, whereby the respective line disclose in column 7 the fine chemical myo-inositol. For example, an increase of the myo-inositol of at least 1 percent, particularly in a range of 21 to 26-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a fatty acid desaturase, or if the activity of the polypeptide Slr1350, preferably represented by SEQ ID NO. 116714, or a homolog or fragment thereof, or if the activity of a polypeptide encoded by a nucleic acid molecule comprising the nucleic acid SEQ ID NO. 116713, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 116713 or polypeptide SEQ ID NO. 116714, respectively, or a homolog or a fragment thereof, is increased or generated, or if the activity fatty acid desaturase is increased or generated in a non-human organism, like a microorganism or a plant cell, plant or part thereof, especially with mitochondrial localization, whereby the respective line disclose in column 7 the fine chemical glucose-1-phosphate. For example, an increase of the glucose-1-phosphate of at least 1 percent, particularly in a range of 26 to 61-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a CDP-diglyceride synthetase, or if the activity of the polypeptide Slr1369, preferably represented by SEQ ID NO. 58473, or a homolog or fragment thereof, or if the activity of a polypeptide encoded by a nucleic acid molecule comprising the nucleic acid SEQ ID NO. 58472, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 58472 or polypeptide SEQ ID NO. 58473, respectively, or a homolog or a fragment thereof, is increased or generated, or if the activity CDP-diglyceride synthetase is increased or generated in a non-human organism, like a microorganism or a plant cell, plant or part thereof, especially with mitochondrial localization, whereby the respective line disclose in column 7 the fine chemical glucose-1-phosphate. For example, an increase of the glucose-1-phosphate of at least 1 percent, particularly in a range of 39 to 63-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a CDP-diglyceride synthetase, or if the activity of the polypeptide Slr1369, preferably represented by SEQ ID NO. 58473, or a homolog or fragment thereof, or if the activity of a polypeptide encoded by a nucleic acid molecule comprising the nucleic acid SEQ ID NO. 58472, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 58472 or polypeptide SEQ ID NO. 58473, respectively, or a homolog or a fragment thereof, is increased or generated, or if the activity CDP-diglyceride synthetase is increased or generated in a non-human organism, like a microorganism or a plant cell, plant or part thereof, especially with mitochondrial localization, whereby the respective line disclose in column 7 the fine chemical myo-inositol. For example, an increase of the myo-inositol of at least 1 percent, particularly in a range of 29 to 62-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a iron(III) dicitrate-binding protein, or if the activity of the polypeptide Slr1492, preferably represented by SEQ ID NO. 58669, or a homolog or fragment thereof, or if the activity of a polypeptide encoded by a nucleic acid molecule comprising the nucleic acid SEQ ID NO. 58668, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 58668 or polypeptide SEQ ID NO. 58669, respectively, or a homolog or a fragment thereof, is increased or generated, or if the activity iron(III) dicitrate-binding protein is increased or generated in a non-human organism, like a microorganism or a plant cell, plant or part thereof, especially with mitochondrial localization, whereby the respective line disclose in column 7 the fine chemical fructose. For example, an increase of the fructose of at least 1 percent, particularly in a range of 82 to 150-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a photosystem I reaction center subunit XI, or if the activity of the polypeptide Slr1655, preferably represented by SEQ ID NO. 12071, or a homolog or fragment thereof, or if the activity of a polypeptide encoded by a nucleic acid molecule comprising the nucleic acid SEQ ID NO. 12070, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 12070 or polypeptide SEQ ID NO. 12071, respectively, or a homolog or a fragment thereof, is increased or generated, or if the activity photosystem I reaction center subunit XI is increased or generated in a non-human organism, like a microorganism or a plant cell, plant or part thereof, especially with mitochondrial localization, whereby the respective line disclose in column 7 the fine chemical sucrose. For example, an increase of the sucrose of at least 1 percent, particularly in a range of 26 to 64-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a photosystem II protein, or if the activity of the polypeptide Slr1739, preferably represented by SEQ ID NO. 58732, or a homolog or fragment thereof, or if the activity of a polypeptide encoded by a nucleic acid molecule comprising the nucleic acid SEQ ID NO. 58731, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 58731 or polypeptide SEQ ID NO. 58732, respectively, or a homolog or a fragment thereof, is increased or generated, or if the activity photosystem II protein is increased or generated in a non-human organism, like a microorganism or a plant cell, plant or part thereof, especially with plastidic localization, whereby the respective line disclose in column 7 the fine chemical fructose. For example, an increase of the fructose of at least 1 percent, particularly in a range of 84 to 407-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a glutamine amidotransferase, or if the activity of the polypeptide Slr1742, preferably represented by SEQ ID NO. 58752, or a homolog or fragment thereof, or if the activity of a polypeptide encoded by a nucleic acid molecule comprising the nucleic acid SEQ ID NO. 58751, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 58751 or polypeptide SEQ ID NO. 58752, respectively, or a homolog or a fragment thereof, is increased or generated, or if the activity glutamine amidotransferase is increased or generated in a non-human organism, like a microorganism or a plant cell, plant or part thereof, especially with plastidic localization, whereby the respective line disclose in column 7 the fine chemical fructose. For example, an increase of the fructose of at least 1 percent, particularly in a range of 76 to 91-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a glutamine amidotransferase, or if the activity of the polypeptide Slr1742, preferably represented by SEQ ID NO. 58752, or a homolog or fragment thereof, or if the activity of a polypeptide encoded by a nucleic acid molecule comprising the nucleic acid SEQ ID NO. 58751, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 58751 or polypeptide SEQ ID NO. 58752, respectively, or a homolog or a fragment thereof, is increased or generated, or if the activity glutamine amidotransferase is increased or generated in a non-human organism, like a microorganism or a plant cell, plant or part thereof, especially with plastidic localization, whereby the respective line disclose in column 7 the fine chemical myo-inositol. For example, an increase of the myo-inositol of at least 1 percent, particularly in a range of 27 to 35-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a NADH dehydrogenase, or if the activity of the polypeptide Slr1743, preferably represented by SEQ ID NO. 147458, or a homolog or fragment thereof, or if the activity of a polypeptide encoded by a nucleic acid molecule comprising the nucleic acid SEQ ID NO. 147457, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 147457 or polypeptide SEQ ID NO. 147458, respectively, or a homolog or a fragment thereof, is increased or generated, or if the activity NADH dehydrogenase is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 28 to 51-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a pyridoxal phosphate biosynthetic protein, or if the activity of the polypeptide Slr1779, preferably represented by SEQ ID NO. 147638, or a homolog or fragment thereof, or if the activity of a polypeptide encoded by a nucleic acid molecule comprising the nucleic acid SEQ ID NO. 147637, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 147637 or polypeptide SEQ ID NO. 147638, respectively, or a homolog or a fragment thereof, is increased or generated, or if the activity pyridoxal phosphate biosynthetic protein is increased or generated in a non-human organism, like a microorganism or a plant cell, plant or part thereof, especially with plastidic localization, whereby the respective line disclose in column 7 the fine chemical raffinose. For example, an increase of the raffinose of at least 1 percent, particularly in a range of 55 to 176-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a phosphoadenosine phosphosulfate reductase , or if the activity of the polypeptide Slr1791, preferably represented by SEQ ID NO. 12141, or a homolog or fragment thereof, or if the activity of a polypeptide encoded by a nucleic acid molecule comprising the nucleic acid SEQ ID NO. 12140, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 12140 or polypeptide SEQ ID NO. 12141, respectively, or a homolog or a fragment thereof, is increased or generated, or if the activity phosphoadenosine phosphosulfate reductase is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 21 to 48-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a threonine dehydratase, or if the activity of the polypeptide Slr2072, preferably represented by SEQ ID NO. 12342, or a homolog or fragment thereof, or if the activity of a polypeptide encoded by a nucleic acid molecule comprising the nucleic acid SEQ ID NO. 12341, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 12341 or polypeptide SEQ ID NO. 12342, respectively, or a homolog or a fragment thereof, is increased or generated, or if the activity threonine dehydratase is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 28 to 82-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a threonine dehydratase, or if the activity of the polypeptide Slr2072, preferably represented by SEQ ID NO. 12342, or a homolog or fragment thereof, or if the activity of a polypeptide encoded by a nucleic acid molecule comprising the nucleic acid SEQ ID NO. 12341, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 12341 or polypeptide SEQ ID NO. 12342, respectively, or a homolog or a fragment thereof, is increased or generated, or if the activity threonine dehydratase is increased or generated in a non-human organism, like a microorganism or a plant cell, plant or part thereof, especially with plastidic localization, whereby the respective line disclose in column 7 the fine chemical 3,4,5-trihydroxypentanoic acid. For example, an increase of the 3,4,5-trihydroxypentanoic acid of at least 1 percent, particularly in a range of 34 to 121-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a short-chain alcohol dehydrogenase family, or if the activity of the polypeptide Slr2124, preferably represented by SEQ ID NO. 59371, or a homolog or fragment thereof, or if the activity of a polypeptide encoded by a nucleic acid molecule comprising the nucleic acid SEQ ID NO. 59370, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 59370 or polypeptide SEQ ID NO. 59371, respectively, or a homolog or a fragment thereof, is increased or generated, or if the activity short-chain alcohol dehydrogenase family is increased or generated in a non-human organism, like a microorganism or a plant cell, plant or part thereof, especially with plastidic localization, whereby the respective line disclose in column 7 the fine chemical fructose. For example, an increase of the fructose of at least 1 percent, particularly in a range of 152 to 214-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a short-chain alcohol dehydrogenase family, or if the activity of the polypeptide Slr2124, preferably represented by SEQ ID NO. 59371, or a homolog or fragment thereof, or if the activity of a polypeptide encoded by a nucleic acid molecule comprising the nucleic acid SEQ ID NO. 59370, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 59370 or polypeptide SEQ ID NO. 59371, respectively, or a homolog or a fragment thereof, is increased or generated, or if the activity short-chain alcohol dehydrogenase family is increased or generated in a non-human organism, like a microorganism or a plant cell, plant or part thereof, especially with plastidic localization, whereby the respective line disclose in column 7 the fine chemical glucose. For example, an increase of the glucose of at least 1 percent, particularly in a range of 123 to 150-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a short-chain alcohol dehydrogenase family, or if the activity of the polypeptide Slr2124, preferably represented by SEQ ID NO. 59371, or a homolog or fragment thereof, or if the activity of a polypeptide encoded by a nucleic acid molecule comprising the nucleic acid SEQ ID NO. 59370, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 59370 or polypeptide SEQ ID NO. 59371, respectively, or a homolog or a fragment thereof, is increased or generated, or if the activity short-chain alcohol dehydrogenase family is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 31 to 31-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a short-chain alcohol dehydrogenase family, or if the activity of the polypeptide Slr2124, preferably represented by SEQ ID NO. 59371, or a homolog or fragment thereof, or if the activity of a polypeptide encoded by a nucleic acid molecule comprising the nucleic acid SEQ ID NO. 59370, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 59370 or polypeptide SEQ ID NO. 59371, respectively, or a homolog or a fragment thereof, is increased or generated, or if the activity short-chain alcohol dehydrogenase family is increased or generated in a non-human organism, like a microorganism or a plant cell, plant or part thereof, especially with plastidic localization, whereby the respective line disclose in column 7 the fine chemical myo-inositol. For example, an increase of the myo-inositol of at least 1 percent, particularly in a range of 24 to 38-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a short-chain alcohol dehydrogenase family, or if the activity of the polypeptide Slr2124, preferably represented by SEQ ID NO. 59371, or a homolog or fragment thereof, or if the activity of a polypeptide encoded by a nucleic acid molecule comprising the nucleic acid SEQ ID NO. 59370, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 59370 or polypeptide SEQ ID NO. 59371, respectively, or a homolog or a fragment thereof, is increased or generated, or if the activity short-chain alcohol dehydrogenase family is increased or generated in a non-human organism, like a microorganism or a plant cell, plant or part thereof, especially with plastidic localization, whereby the respective line disclose in column 7 the fine chemical sucrose. For example, an increase of the sucrose of at least 1 percent, particularly in a range of 24 to 45-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a phosphate acetyltransferase, or if the activity of the polypeptide Slr2132, preferably represented by SEQ ID NO. 147837, or a homolog or fragment thereof, or if the activity of a polypeptide encoded by a nucleic acid molecule comprising the nucleic acid SEQ ID NO. 147836, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 147836 or polypeptide SEQ ID NO. 147837, respectively, or a homolog or a fragment thereof, is increased or generated, or if the activity phosphate acetyltransferase is increased or generated in a non-human organism, like a microorganism or a plant cell, plant or part thereof, especially with mitochondrial localization, whereby the respective line disclose in column 7 the fine chemical raffinose. For example, an increase of the raffinose of at least 1 percent, particularly in a range of 75 to 205-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a photosystem I reaction centre subunit, or if the activity of the polypeptide Smr0004, preferably represented by SEQ ID NO. 136908, or a homolog or fragment thereof, or if the activity of a polypeptide encoded by a nucleic acid molecule comprising the nucleic acid SEQ ID NO. 136907, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 136907 or polypeptide SEQ ID NO. 136908, respectively, or a homolog or a fragment thereof, is increased or generated, or if the activity photosystem I reaction centre subunit is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 31 to 52-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a photosystem I reaction centre subunit, or if the activity of the polypeptide Smr0004, preferably represented by SEQ ID NO. 136908, or a homolog or fragment thereof, or if the activity of a polypeptide encoded by a nucleic acid molecule comprising the nucleic acid SEQ ID NO. 136907, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 136907 or polypeptide SEQ ID NO. 136908, respectively, or a homolog or a fragment thereof, is increased or generated, or if the activity photosystem I reaction centre subunit is increased or generated in a non-human organism, like a microorganism or a plant cell, plant or part thereof, especially with plastidic localization, whereby the respective line disclose in column 7 the fine chemical raffinose. For example, an increase of the raffinose of at least 1 percent, particularly in a range of 79 to 166-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a photosystem I reaction centre subunit, or if the activity of the polypeptide Smr0004, preferably represented by SEQ ID NO. 136908, or a homolog or fragment thereof, or if the activity of a polypeptide encoded by a nucleic acid molecule comprising the nucleic acid SEQ ID NO. 136907, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 136907 or polypeptide SEQ ID NO. 136908, respectively, or a homolog or a fragment thereof, is increased or generated, or if the activity photosystem I reaction centre subunit is increased or generated in a non-human organism, like a microorganism or a plant cell, plant or part thereof, especially with plastidic localization, whereby the respective line disclose in column 7 the fine chemical 3,4,5-trihydroxypentanoic acid. For example, an increase of the 3,4,5-trihydroxypentanoic acid of at least 1 percent, particularly in a range of 33 to 78-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a Sec-independent protein translocase subunit, or if the activity of the polypeptide TTC0019, preferably represented by SEQ ID NO. 12699, or a homolog or fragment thereof, or if the activity of a polypeptide encoded by a nucleic acid molecule comprising the nucleic acid SEQ ID NO. 12698, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 12698 or polypeptide SEQ ID NO. 12699, respectively, or a homolog or a fragment thereof, is increased or generated, or if the activity Sec-independent protein translocase subunit is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 83 to 4740-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a Sec-independent protein translocase subunit, or if the activity of the polypeptide TTC0019, preferably represented by SEQ ID NO. 12699, or a homolog or fragment thereof, or if the activity of a polypeptide encoded by a nucleic acid molecule comprising the nucleic acid SEQ ID NO. 12698, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 12698 or polypeptide SEQ ID NO. 12699, respectively, or a homolog or a fragment thereof, is increased or generated, or if the activity Sec-independent protein translocase subunit is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 62 to 1517-percent is conferred as compared to a corresponding non-transformed wild type non-human organism.

Accordingly, in one embodiment, an increase of sedoheptulose-7-phosphate in a non-human organism, as compared to a corresponding non-transformed wild type non-human organism, is conferred in the process of the invention, if the activity of a polypeptide showing the activity of a Sec-independent protein translocase subunit, or if the activity of the polypeptide TTC0019, preferably represented by SEQ ID NO. 12699, or a homolog or fragment thereof, or if the activity of a polypeptide encoded by a nucleic acid molecule comprising the nucleic acid SEQ ID NO. 12698, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 12698 or polypeptide SEQ ID NO. 12699, respectively, or a homolog or a fragment thereof, is increased or generated, or if the activity Sec-independent protein translocase subunit is increased or generated in a non-human organism, like a microorganism or a plant cell, plant or part thereof, especially with non-targeted localization, whereby the respective line disclose in column 7 the fine chemical sedoheptulose-7-phosphate. For example, an increase of the sedoheptulose-7-phosphate of at least 1 percent, particularly in a range of 57 to 296-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a cell division protein, or if the activity of the polypeptide TTC0035, preferably represented by SEQ ID NO. 60302, or a homolog or fragment thereof, or if the activity of a polypeptide encoded by a nucleic acid molecule comprising the nucleic acid SEQ ID NO. 60301, preferably the coding region thereof, or a homolog or fragment thereof, e.g. derived from Thermus thermophilus, is increased or generated. For example the activity of a nucleic acid molecule or a polypeptide comprising the nuoleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 60301 or polypeptide SEQ ID NO. 60302, respectively, or a homolog or a fragment thereof, is increased or generated, or if the activity cell division protein is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 77 to 154-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a cell division protein, or if the activity of the polypeptide TTC0035, preferably represented by SEQ ID NO. 60302, or a homolog or fragment thereof, or if the activity of a polypeptide encoded by a nucleic acid molecule comprising the nucleic acid SEQ ID NO. 60301, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 60301 or polypeptide SEQ ID NO. 60302, respectively, or a homolog or a fragment thereof, is increased or generated, or if the activity cell division protein is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 51 to 84-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a cell division protein, or if the activity of the polypeptide TTC0035, preferably represented by SEQ ID NO. 60302, or a homolog or fragment thereof, or if the activity of a polypeptide encoded by a nucleic acid molecule comprising the nucleic acid SEQ ID NO. 60301, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 60301 or polypeptide SEQ ID NO. 60302, respectively, or a homolog or a fragment thereof, is increased or generated, or if the activity cell division protein is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 27 to 34-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a branched-chain amino acid ABC transporter permease protein, or if the activity of the polypeptide TTC0216, preferably represented by SEQ ID NO. 60860, or a homolog or fragment thereof, or if the activity of a polypeptide encoded by a nucleic acid molecule comprising the nucleic acid SEQ ID NO. 60859, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 60859 or polypeptide SEQ ID NO. 60860, respectively, or a homolog or a fragment thereof, is increased or generated, or if the activity branched-chain amino acid ABC transporter permease protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 32 to 117 -percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a branched-chain amino acid ABC transporter permease protein, or if the activity of the polypeptide TTC0216, preferably represented by SEQ ID NO. 60860, or a homolog or fragment thereof, or if the activity of a polypeptide encoded by a nucleic acid molecule comprising the nucleic acid SEQ ID NO. 60859, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 60859 or polypeptide SEQ ID NO. 60860, respectively, or a homolog or a fragment thereof, is increased or generated, or if the activity branched-chain amino acid ABC transporter permease protein is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 46 to 95-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a 3-isopropylmalate dehydratase large subunit, or if the activity of the polypeptide TTC0865, preferably represented by SEQ ID NO. 147945, or a homolog or fragment thereof, or if the activity of a polypeptide encoded by a nucleic acid molecule comprising the nucleic acid SEQ ID NO. 147944, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 147944 or polypeptide SEQ ID NO. 147945, respectively, or a homolog or a fragment thereof, is increased or generated, or if the activity 3-isopropylmalate dehydratase large subunit is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 30 to 49-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a multiple antibiotic resistance protein, or if the activity of the polypeptide TTC1193, preferably represented by SEQ ID NO. 61724, or a homolog or fragment thereof, or if the activity of a polypeptide encoded by a nucleic acid molecule comprising the nucleic acid SEQ ID NO. 61723, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 61723 or polypeptide SEQ ID NO. 61724, respectively, or a homolog or a fragment thereof, is increased or generated, or if the activity multiple antibiotic resistance protein is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 88 to 1786-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a multiple antibiotic resistance protein, or if the activity of the polypeptide TTC1193, preferably represented by SEQ ID NO. 61724, or a homolog or fragment thereof, or if the activity of a polypeptide encoded by a nucleic acid molecule comprising the nucleic acid SEQ ID NO. 61723, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 61723 or polypeptide SEQ ID NO. 61724, respectively, or a homolog or a fragment thereof, is increased or generated, or if the activity multiple antibiotic resistance protein is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 63 to 1072-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a multiple antibiotic resistance protein, or if the activity of the polypeptide TTC1193, preferably represented by SEQ ID NO. 61724, or a homolog or fragment thereof, or if the activity of a polypeptide encoded by a nucleic acid molecule comprising the nucleic acid SEQ ID NO. 61723, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 61723 or polypeptide SEQ ID NO. 61724, respectively, or a homolog or a fragment thereof, is increased or generated, or if the activity multiple antibiotic resistance protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 38 to 77-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a TTC1386-protein, or if the activity of the polypeptide TTC1386, preferably represented by SEQ ID NO. 62080, or a homolog or fragment thereof, or if the activity of a polypeptide encoded by a nucleic acid molecule comprising the nucleic acid SEQ ID NO. 62079, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 62079 or polypeptide SEQ ID NO. 62080, respectively, or a homolog or a fragment thereof, is increased or generated, or if the activity TTC1386-protein is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 363 to 1435-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a TTC1386-protein, or if the activity of the polypeptide TTC1386, preferably represented by SEQ ID NO. 62080, or a homolog or fragment thereof, or if the activity of a polypeptide encoded by a nucleic acid molecule comprising the nucleic acid SEQ ID NO. 62079, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 62079 or polypeptide SEQ ID NO. 62080, respectively, or a homolog or a fragment thereof, is increased or generated, or if the activity TTC1386-protein is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 197 to 296-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a homocitrate synthase, or if the activity of the polypeptide TTC1550, preferably represented by SEQ ID NO. 12975, or a homolog or fragment thereof, or if the activity of a polypeptide encoded by a nucleic acid molecule comprising the nucleic acid SEQ ID NO. 12974, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 12974 or polypeptide SEQ ID NO. 12975, respectively, or a homolog or a fragment thereof, is increased or generated, or if the activity homocitrate synthase is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 128 to 915-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a homocitrate synthase, or if the activity of the polypeptide TTC1550, preferably represented by SEQ ID NO. 12975, or a homolog or fragment thereof, or if the activity of a polypeptide encoded by a nucleic acid molecule comprising the nucleic acid SEQ ID NO. 12974, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 12974 or polypeptide SEQ ID NO. 12975, respectively, or a homolog or a fragment thereof, is increased or generated, or if the activity homocitrate synthase is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 19 to 53-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a homocitrate synthase, or if the activity of the polypeptide TTC1550, preferably represented by SEQ ID NO. 12975, or a homolog or fragment thereof, or if the activity of a polypeptide encoded by a nucleic acid molecule comprising the nucleic acid SEQ ID NO. 12974, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 12974 or polypeptide SEQ ID NO. 12975, respectively, or a homolog or a fragment thereof, is increased or generated, or if the activity homocitrate synthase is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 24 to 83-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a permease, or if the activity of the polypeptide TTC1893, preferably represented by SEQ ID NO. 148560, or a homolog or fragment thereof, or if the activity of a polypeptide encoded by a nucleic acid molecule comprising the nucleic acid SEQ ID NO. 148559, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 148559 or polypeptide SEQ ID NO. 148560, respectively, or a homolog or a fragment thereof, is increased or generated, or if the activity permease is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 66 to 72-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a permease, or if the activity of the polypeptide TTC1893, preferably represented by SEQ ID NO. 148560, or a homolog or fragment thereof, or if the activity of a polypeptide encoded by a nucleic acid molecule comprising the nucleic acid SEQ ID NO. 148559, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 148559 or polypeptide SEQ ID NO. 148560, respectively, or a homolog or a fragment thereof, is increased or generated, or if the activity permease is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 47 to 62-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a oxidoreductase subunit, or if the activity of the polypeptide TTC1918, preferably represented by SEQ ID NO. 62161, or a homolog or fragment thereof, or if the activity of a polypeptide encoded by a nucleic acid molecule comprising the nucleic acid SEQ ID NO. 62160, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 62160 or polypeptide SEQ ID NO. 62161, respectively, or a homolog or a fragment thereof, is increased or generated, or if the activity oxidoreductase subunit is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 21 to 42-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a XM_{—}473199-protein, or if the activity of the polypeptide XM_{—}473199, preferably represented by SEQ ID NO. 62245, or a homolog or fragment thereof, or if the activity of a polypeptide encoded by a nucleic acid molecule comprising the nucleic acid SEQ ID NO. 62244, preferably the coding region thereof, or a homolog or fragment thereof, e.g. derived from Oryza sativa, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 62244 or polypeptide SEQ ID NO. 62245, respectively, or a homolog or a fragment thereof, is increased or generated, or if the activity XM_{—}473199-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 18 to 54-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a transcription factor, or if the activity of the polypeptide Ybl021c, preferably represented by SEQ ID NO. 62525, or a homolog or fragment thereof, or if the activity of a polypeptide encoded by a nucleic acid molecule comprising the nucleic acid SEQ ID NO. 62524, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 62524 or polypeptide SEQ ID NO. 62525, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 26 to 84-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a transcription factor, or if the activity of the polypeptide Ybl021 c, preferably represented by SEQ ID NO. 62525, or a homolog or fragment thereof, or if the activity of a polypeptide encoded by a nucleic acid molecule comprising the nucleic acid SEQ ID NO. 62524, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 62524 or polypeptide SEQ ID NO. 62525, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 25 to 53-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a 3-deoxy-7-phosphoheptulonate synthase, or if the activity of the polypeptide Ybr249c, preferably represented by SEQ ID NO. 88880, or a homolog or fragment thereof, or if the activity of a polypeptide encoded by a nucleic acid molecule comprising the nucleic acid SEQ ID NO. 88879, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 88879 or polypeptide SEQ ID NO. 88880, respectively, or a homolog or a fragment thereof, is increased or generated, or if the activity 3-deoxy-7-phosphoheptulonate synthase is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 32 to 59-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a phosphate permease, or if the activity of the polypeptide Ybr296c, preferably represented by SEQ ID NO. 89318, or a homolog or fragment thereof, or if the activity of a polypeptide encoded by a nucleic acid molecule comprising the nucleic acid SEQ ID NO. 89317, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 89317 or polypeptide SEQ ID NO. 89318, respectively, or a homolog or a fragment thereof, is increased or generated, or if the activity phosphate permease is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 35 to 46-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a phosphate permease, or if the activity of the polypeptide Ybr296c, preferably represented by SEQ ID NO. 89318, or a homolog or fragment thereof, or if the activity of a polypeptide encoded by a nucleic acid molecule comprising the nucleic acid SEQ ID NO. 89317, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 89317 or polypeptide SEQ ID NO. 89318, respectively, or a homolog or a fragment thereof, is increased or generated, or if the activity phosphate permease is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 37 to 53-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a ycl027w-protein, or if the activity of the polypeptide Ycl027w, preferably represented by SEQ ID NO. 96981, or a homolog or fragment thereof, or if the activity of a polypeptide encoded by a nucleic acid molecule comprising the nucleic acid SEQ ID NO. 96980, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 96980 or polypeptide SEQ ID NO. 96981, respectively, or a homolog or a fragment thereof, is increased or generated, or if the activity ycl027w-protein is increased or generated in a non-human organism, like a microorganism or a plant cell, plant or part thereof, especially with plastidic localization, whereby the respective line disclose in column 7 the fine chemical fructose. For example, an increase of the fructose of at least 1 percent, particularly in a range of 61 to 266-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a ycl027w-protein, or if the activity of the polypeptide Ycl027w, preferably represented by SEQ ID NO. 96981, or a homolog or fragment thereof, or if the activity of a polypeptide encoded by a nucleic acid molecule comprising the nucleic acid SEQ ID NO. 96980, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 96980 or polypeptide SEQ ID NO. 96981, respectively, or a homolog or a fragment thereof, is increased or generated, or if the activity ycl027w-protein is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 35 to 66-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a serine/threonine dehydratase, or if the activity of the polypeptide Ycl064c, preferably represented by SEQ ID NO. 89396, or a homolog or fragment thereof, or if the activity of a polypeptide encoded by a nucleic acid molecule comprising the nucleic acid SEQ ID NO. 89395, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 89395 or polypeptide SEQ ID NO. 89396, respectively, or a homolog or a fragment thereof, is increased or generated, or if the activity serine/threonine dehydratase is increased or generated in a non-human organism, like a microorganism or a plant cell, plant or part thereof, especially with plastidic localization, whereby the respective line disclose in column 7 the fine chemical myo-inositol. For example, an increase of the myo-inositol of at least 1 percent, particularly in a range of 28 to 48-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a serine/threonine dehydratase, or if the activity of the polypeptide Ycl064c, preferably represented by SEQ ID NO. 89396, or a homolog or fragment thereof, or if the activity of a polypeptide encoded by a nucleic acid molecule comprising the nucleic acid SEQ ID NO. 89395, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 89395 or polypeptide SEQ ID NO. 89396, respectively, or a homolog or a fragment thereof, is increased or generated, or if the activity serine/threonine dehydratase is increased or generated in a non-human organism, like a microorganism or a plant cell, plant or part thereof, especially with plastidic localization, whereby the respective line disclose in column 7 the fine chemical sucrose. For example, an increase of the sucrose of at least 1 percent, particularly in a range of 37 to 37-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a phosphoglycerate kinase, or if the activity of the polypeptide Ycr012w, preferably represented by SEQ ID NO. 108114, or a homolog or fragment thereof, or if the activity of a polypeptide encoded by a nucleic acid molecule comprising the nucleic acid SEQ ID NO. 108113, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 108113 or polypeptide SEQ ID NO. 108114, respectively, or a homolog or a fragment thereof, is increased or generated, or if the activity phosphoglycerate kinase is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 83 to 188-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a phosphoglycerate kinase, or if the activity of the polypeptide Ycr012w, preferably represented by SEQ ID NO. 108114, or a homolog or fragment thereof, or if the activity of a polypeptide encoded by a nucleic acid molecule comprising the nucleic acid SEQ ID NO. 108113, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 108113 or polypeptide SEQ ID NO. 108114, respectively, or a homolog or a fragment thereof, is increased or generated, or if the activity phosphoglycerate kinase is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 81 to 181-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a YCR047W-A-protein, or if the activity of the polypeptide Ycr047w-a, preferably represented by SEQ ID NO. 148580, or a homolog or fragment thereof, or if the activity of a polypeptide encoded by a nucleic acid molecule comprising the nucleic acid SEQ ID NO. 148579, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 148579 or polypeptide SEQ ID NO. 148580, respectively, or a homolog or a fragment thereof, is increased or generated, or if the activity YCR047W-A-protein is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 69 to 234-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a ycr102c-protein, or if the activity of the polypeptide Ycr102c, preferably represented by SEQ ID NO. 63265, or a homolog or fragment thereof, or if the activity of a polypeptide encoded by a nucleic acid molecule comprising the nucleic acid SEQ ID NO. 63264, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 63264 or polypeptide SEQ ID NO. 63265, respectively, or a homolog or a fragment thereof, is increased or generated, or if the activity ycr102c-protein is increased or generated in a non-human organism, like a microorganism or a plant cell, plant or part thereof, especially with plastidic localization, whereby the respective line disclose in column 7 the fine chemical 3,4,5-trihydroxypentanoic acid. For example, an increase of the 3,4,5-trihydroxypentanoic acid of at least 1 percent, particularly in a range of 39 to 87-percent is conferred as compared to a corresponding non-transformed wild type non-human organism.

Accordingly, in one embodiment, an increase of galactinol in a non-human organism, as compared to a corresponding non-transformed wild type non-human organism, is conferred in the process of the invention, if the activity of a polypeptide showing the activity of a isocitrate dehydrogenase, or if the activity of the polypeptide Ydl066w, preferably represented by SEQ ID NO. 122229, or a homolog or fragment thereof, or if the activity of a polypeptide encoded by a nucleic acid molecule comprising the nucleic acid SEQ ID NO. 122228, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 122228 or polypeptide SEQ ID NO. 122229, respectively, or a homolog or a fragment thereof, is increased or generated, or if the activity isocitrate dehydrogenase is increased or generated in a non-human organism, like a microorganism or a plant cell, plant or part thereof, especially with non-targeted localization, whereby the respective line disclose in column 7 the fine chemical galactinol. For example, an increase of the galactinol of at least 1 percent, particularly in a range of 37 to 61-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a cell division control protein, or if the activity of the polypeptide Ydl126c, preferably represented by SEQ ID NO. 120046, or a homolog or fragment thereof, or if the activity of a polypeptide encoded by a nucleic acid molecule comprising the nucleic acid SEQ ID NO. 120045, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 120045 or polypeptide SEQ ID NO. 120046, respectively, or a homolog or a fragment thereof, is increased or generated, or if the activity cell division control protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 24 to 35-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a cyclin, or if the activity of the polypeptide Ydl155w, preferably represented by SEQ ID NO. 14172, or a homolog or fragment thereof, or if the activity of a polypeptide encoded by a nucleic acid molecule comprising the nucleic acid SEQ ID NO. 14171, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 14171 or polypeptide SEQ ID NO. 14172, respectively, or a homolog or a fragment thereof, is increased or generated, or if the activity cyclin is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 33 to 112-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a YDL159W-A-protein, or if the activity of the polypeptide Ydl159w-a, preferably represented by SEQ ID NO. 137499, or a homolog or fragment thereof, or if the activity of a polypeptide encoded by a nucleic acid molecule comprising the nucleic acid SEQ ID NO. 137498, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 137498 or polypeptide SEQ ID NO. 137499, respectively, or a homolog or a fragment thereof, is increased or generated, or if the activity YDL159W-A-protein is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 57 to 204-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a protein kinase, or if the activity of the polypeptide Ydl168w, preferably represented by SEQ ID NO. 14303, or a homolog or fragment thereof, or if the activity of a polypeptide encoded by a nucleic acid molecule comprising the nucleic acid SEQ ID NO. 14302, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 14302 or polypeptide SEQ ID NO. 14303, respectively, or a homolog or a fragment thereof, is increased or generated, or if the activity protein kinase is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 34 to 126-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a serine/threonine-protein phosphatase, or if the activity of the polypeptide Ydl188c, preferably represented by SEQ ID NO. 89484, or a homolog or fragment thereof, or if the activity of a polypeptide encoded by a nucleic acid molecule comprising the nucleic acid SEQ ID NO. 89483, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 89483 or polypeptide SEQ ID NO. 89484, respectively, or a homolog or a fragment thereof, is increased or generated, or if the activity serine/threonine-protein phosphatase is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 40 to 58-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a integral membrane protein, or if the activity of the polypeptide YDL222C, preferably represented by SEQ ID NO. 148584, or a homolog or fragment thereof, or if the activity of a polypeptide encoded by a nucleic acid molecule comprising the nucleic acid SEQ ID NO. 148583, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 148583 or polypeptide SEQ ID NO. 148584, respectively, or a homolog or a fragment thereof, is increased or generated, or if the activity integral membrane protein is increased or generated in a non-human organism, like a microorganism or a plant cell, plant or part thereof, especially with plastidic localization, whereby the respective line disclose in column 7 the fine chemical 3,4,5-trihydroxypentanoic acid. For example, an increase of the 3,4,5-trihydroxypentanoic acid of at least 1 percent, particularly in a range of 36 to 79-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a ydl235c-protein, or if the activity of the polypeptide Ydl235c, preferably represented by SEQ ID NO. 113314, or a homolog or fragment thereof, or if the activity of a polypeptide encoded by a nucleic acid molecule comprising the nucleic acid SEQ ID NO. 113313, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 113313 or polypeptide SEQ ID NO. 113314, respectively, or a homolog or a fragment thereof, is increased or generated, or if the activity ydl235c-protein is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 31 to 88-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a ydl235c-protein, or if the activity of the polypeptide Ydl235c, preferably represented by SEQ ID NO. 113314, or a homolog or fragment thereof, or if the activity of a polypeptide encoded by a nucleic acid molecule comprising the nucleic acid SEQ ID NO. 113313, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 113313 or polypeptide SEQ ID NO. 113314, respectively, or a homolog or a fragment thereof, is increased or generated, or if the activity ydl235c-protein is increased or generated in a non-human organism, like a microorganism or a plant cell, plant or part thereof, especially with plastidic localization, whereby the respective line disclose in column 7 the fine chemical myo-inositol. For example, an increase of the myo-inositol of at least 1 percent, particularly in a range of 30 to 103-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a ydl235c-protein, or if the activity of the polypeptide Ydl235c, preferably represented by SEQ ID NO. 113314, or a homolog or fragment thereof, or if the activity of a polypeptide encoded by a nucleic acid molecule comprising the nucleic acid SEQ ID NO. 113313, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 113313 or polypeptide SEQ ID NO. 113314, respectively, or a homolog or a fragment thereof, is increased or generated, or if the activity ydl235c-protein is increased or generated in a non-human organism, like a microorganism or a plant cell, plant or part thereof, especially with plastidic localization, whereby the respective line disclose in column 7 the fine chemical 3,4,5-trihydroxypentanoic acid. For example, an increase of the 3,4,5-trihydroxypentanoic acid of at least 1 percent, particularly in a range of 48 to 88-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a coproporphyrinogen III oxidase, or if the activity of the polypeptide Ydr044w, preferably represented by SEQ ID NO. 63335, or a homolog or fragment thereof, or if the activity of a polypeptide encoded by a nucleic acid molecule comprising the nucleic acid SEQ ID NO. 63334, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 63334 or polypeptide SEQ ID NO. 63335, respectively, or a homolog or a fragment thereof, is increased or generated, or if the activity coproporphyrinogen III oxidase is increased or generated in a non-human organism, like a microorganism or a plant cell, plant or part thereof, especially with plastidic localization, whereby the respective line disclose in column 7 the fine chemical sucrose. For example, an increase of the sucrose of at least 1 percent, particularly in a range of 31 to 77-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a branched-chain amino acid permease, or if the activity of the polypeptide Ydr046c, preferably represented by SEQ ID NO. 63545, or a homolog or fragment thereof, or if the activity of a polypeptide encoded by a nucleic acid molecule comprising the nucleic acid SEQ ID NO. 63544, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 63544 or polypeptide SEQ ID NO. 63545, respectively, or a homolog or a fragment thereof, is increased or generated, or if the activity branched-chain amino acid permease is increased or generated in a non-human organism, like a microorganism or a plant cell, plant or part thereof, especially with plastidic localization, whereby the respective line disclose in column 7 the fine chemical glucose. For example, an increase of the glucose of at least 1 percent, particularly in a range of 36 to 43-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a ydr183w-protein, or if the activity of the polypeptide Ydr183w, preferably represented by SEQ ID NO. 90104, or a homolog or fragment thereof, or if the activity of a polypeptide encoded by a nucleic acid molecule comprising the nucleic acid SEQ ID NO. 90103, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 90103 or polypeptide SEQ ID NO. 90104, respectively, or a homolog or a fragment thereof, is increased or generated, or if the activity ydr183w-protein is increased or generated in a non-human organism, like a microorganism or a plant cell, plant or part thereof, especially with plastidic localization, whereby the respective line disclose in column 7 the fine chemical fructose. For example, an increase of the fructose of at least 1 percent, particularly in a range of 158 to 249-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a ydr183w-protein, or if the activity of the polypeptide Ydr183w, preferably represented by SEQ ID NO. 90104, or a homolog or fragment thereof, or if the activity of a polypeptide encoded by a nucleic acid molecule comprising the nucleic acid SEQ ID NO. 90103, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 90103 or polypeptide SEQ ID NO. 90104, respectively, or a homolog or a fragment thereof, is increased or generated, or if the activity ydr183w-protein is increased or generated in a non-human organism, like a microorganism or a plant cell, plant or part thereof, especially with plastidic localization, whereby the respective line disclose in column 7 the fine chemical glucose. For example, an increase of the glucose of at least 1 percent, particularly in a range of 60 to 74-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a ydr507c-protein, or if the activity of the polypeptide Ydr507c, preferably represented by SEQ ID NO. 90166, or a homolog or fragment thereof, or if the activity of a polypeptide encoded by a nucleic acid molecule comprising the nucleic acid SEQ ID NO. 90165, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 90165 or polypeptide SEQ ID NO. 90166, respectively, or a homolog or a fragment thereof, is increased or generated, or if the activity ydr507c-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 18 to 88-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a 1,4-alpha-glucan branching enzyme, or if the activity of the polypeptide Yel011w, preferably represented by SEQ ID NO. 148615, or a homolog or fragment thereof, or if the activity of a polypeptide encoded by a nucleic acid molecule comprising the nucleic acid SEQ ID NO. 148614, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 148614 or polypeptide SEQ ID NO. 148615, respectively, or a homolog or a fragment thereof, is increased or generated, or if the activity 1,4-alpha-glucan branching enzyme is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 113 to 539-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a 1,4-alpha-glucan branching enzyme, or if the activity of the polypeptide Yel011w, preferably represented by SEQ ID NO. 148615, or a homolog or fragment thereof, or if the activity of a polypeptide encoded by a nucleic acid molecule comprising the nucleic acid SEQ ID NO. 148614, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 148614 or polypeptide SEQ ID NO. 148615, respectively, or a homolog or a fragment thereof, is increased or generated, or if the activity 1,4-alpha-glucan branching enzyme is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 71 to 431-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a YEL030C-A-protein, or if the activity of the polypeptide Yel030c-a, preferably represented by SEQ ID NO. 97295, or a homolog or fragment thereof, or if the activity of a polypeptide encoded by a nucleic acid molecule comprising the nucleic acid SEQ ID NO. 97294, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 97294 or polypeptide SEQ ID NO. 97295, respectively, or a homolog or a fragment thereof, is increased or generated, or if the activity YEL030C-A-protein is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 33 to 87-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a aldolase, or if the activity of the polypeptide Yel046c, preferably represented by SEQ ID NO. 128512, or a homolog or fragment thereof, or if the activity of a polypeptide encoded by a nucleic acid molecule comprising the nucleic acid SEQ ID NO. 128511, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 128511 or polypeptide SEQ ID NO. 128512, respectively, or a homolog or a fragment thereof, is increased or generated, or if the activity aldolase is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 21 to 112-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a O-acetyltransferase, or if the activity of the polypeptide Yer024w, preferably represented by SEQ ID NO. 148812, or a homolog or fragment thereof, or if the activity of a polypeptide encoded by a nucleic acid molecule comprising the nucleic acid SEQ ID NO. 148811, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 148811 or polypeptide SEQ ID NO. 148812, respectively, or a homolog or a fragment thereof, is increased or generated, or if the activity O-acetyltransferase is increased or generated in a non-human organism, like a microorganism or a plant cell, plant or part thereof, especially with plastidic localization, whereby the respective line disclose in column 7 the fine chemical glucose. For example, an increase of the glucose of at least 1 percent, particularly in a range of 47 to 92-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a yer177w-protein, or if the activity of the polypeptide Yer177w, preferably represented by SEQ ID NO. 148834, or a homolog or fragment thereof, or if the activity of a polypeptide encoded by a nucleic acid molecule comprising the nucleic acid SEQ ID NO. 148833, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 148833 or polypeptide SEQ ID NO. 148834, respectively, or a homolog or a fragment thereof, is increased or generated, or if the activity yer177w-protein is increased or generated in a non-human organism, like a microorganism or a plant cell, plant or part thereof, especially with plastidic localization, whereby the respective line disclose in column 7 the fine chemical raffinose. For example, an increase of the raffinose of at least 1 percent, particularly in a range of 112 to 148-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a YFL019C-protein, or if the activity of the polypeptide Yfl019c, preferably represented by SEQ ID NO. 64145, or a homolog or fragment thereof, or if the activity of a polypeptide encoded by a nucleic acid molecule comprising the nucleic acid SEQ ID NO. 64144, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 64144 or polypeptide SEQ ID NO. 64145, respectively, or a homolog or a fragment thereof, is increased or generated, or if the activity YFL019C-protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 28 to 275-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a YFL019C-protein, or if the activity of the polypeptide Yfl019c, preferably represented by SEQ ID NO. 64145, or a homolog or fragment thereof, or if the activity of a polypeptide encoded by a nucleic acid molecule comprising the nucleic acid SEQ ID NO. 64144, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 64144 or polypeptide SEQ ID NO. 64145, respectively, or a homolog or a fragment thereof, is increased or generated, or if the activity YFL019C-protein is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 26 to 126-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a hexokinase, or if the activity of the polypeptide Yfr053c, preferably represented by SEQ ID NO. 149220, or a homolog or fragment thereof, or if the activity of a polypeptide encoded by a nucleic acid molecule comprising the nucleic acid SEQ ID NO. 149219, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 149219 or polypeptide SEQ ID NO. 149220, respectively, or a homolog or a fragment thereof, is increased or generated, or if the activity hexokinase is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 27 to 45-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a oxidoreductase, or if the activity of the polypeptide Ygl039w, preferably represented by SEQ ID NO. 120437, or a homolog or fragment thereof, or if the activity of a polypeptide encoded by a nucleic acid molecule comprising the nucleic acid SEQ ID NO. 120436, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 120436 or polypeptide SEQ ID NO. 120437, respectively, or a homolog or a fragment thereof, is increased or generated, or if the activity oxidoreductase is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 30 to 57-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a oxidoreductase, or if the activity of the polypeptide Ygl039w, preferably represented by SEQ ID NO. 120437, or a homolog or fragment thereof, or if the activity of a polypeptide encoded by a nucleic acid molecule comprising the nucleic acid SEQ ID NO. 120436, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 120436 or polypeptide SEQ ID NO. 120437, respectively, or a homolog or a fragment thereof, is increased or generated, or if the activity oxidoreductase is increased or generated in a non-human organism, like a microorganism or a plant cell, plant or part thereof, especially with plastidic localization, whereby the respective line disclose in column 7 the fine chemical 3,4,5-trihydroxypentanoic acid. For example, an increase of the 3,4,5-trihydroxypentanoic acid of at least 1 percent, particularly in a range of 36 to 92-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a ygl096w-protein, or if the activity of the polypeptide Ygl096w, preferably represented by SEQ ID NO. 90279, or a homolog or fragment thereof, or if the activity of a polypeptide encoded by a nucleic acid molecule comprising the nucleic acid SEQ ID NO. 90278, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 90278 or polypeptide SEQ ID NO. 90279, respectively, or a homolog or a fragment thereof, is increased or generated, or if the activity ygl096w-protein is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 18 to 48-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a ygl096w-protein, or if the activity of the polypeptide Ygl096w, preferably represented by SEQ ID NO. 90279, or a homolog or fragment thereof, or if the activity of a polypeptide encoded by a nucleic acid molecule comprising the nucleic acid SEQ ID NO. 90278, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 90278 or polypeptide SEQ ID NO. 90279, respectively, or a homolog or a fragment thereof, is increased or generated, or if the activity ygl096w-protein is increased or generated in a non-human organism, like a microorganism or a plant cell, plant or part thereof, especially with plastidic localization, whereby the respective line disclose in column 7 the fine chemical 3,4,5-trihydroxypentanoic acid. For example, an increase of the 3,4,5-trihydroxypentanoic acid of at least 1 percent, particularly in a range of 33 to 86-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a ygl237c-protein, or if the activity of the polypeptide Ygl237c, preferably represented by SEQ ID NO. 64178, or a homolog or fragment thereof, or if the activity of a polypeptide encoded by a nucleic acid molecule comprising the nucleic acid SEQ ID NO. 64177, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 64177 or polypeptide SEQ ID NO. 64178, respectively, or a homolog or a fragment thereof, is increased or generated, or if the activity ygl237c-protein is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 68 to 290-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a alcohol dehydrogenase, or if the activity of the polypeptide Ygl256w, preferably represented by SEQ ID NO. 130743, or a homolog or fragment thereof, or if the activity of a polypeptide encoded by a nucleic acid molecule comprising the nucleic acid SEQ ID NO. 130742, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 130742 or polypeptide SEQ ID NO. 130743, respectively, or a homolog or a fragment thereof, is increased or generated, or if the activity alcohol dehydrogenase is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 44 to 105-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a alcohol dehydrogenase, or if the activity of the polypeptide Ygl256w, preferably represented by SEQ ID NO. 130743, or a homolog or fragment thereof, or if the activity of a polypeptide encoded by a nucleic acid molecule comprising the nucleic acid SEQ ID NO. 130742, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 130742 or polypeptide SEQ ID NO. 130743, respectively, or a homolog or a fragment thereof, is increased or generated, or if the activity alcohol dehydrogenase is increased or generated in a non-human organism, like a microorganism or a plant cell, plant or part thereof, especially with plastidic localization, whereby the respective line disclose in column 7 the fine chemical 3,4,5-trihydroxypentanoic acid. For example, an increase of the 3,4,5-trihydroxypentanoic acid of at least 1 percent, particularly in a range of 40 to 101-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a yhl013c-protein, or if the activity of the polypeptide Yhl013c, preferably represented by SEQ ID NO. 14716, or a homolog or fragment thereof, or if the activity of a polypeptide encoded by a nucleic acid molecule comprising the nucleic acid SEQ ID NO. 14715, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 14715 or polypeptide SEQ ID NO. 14716, respectively, or a homolog or a fragment thereof, is increased or generated, or if the activity yhl013c-protein is increased or generated in a non-human organism, like a microorganism or a plant cell, plant or part thereof, especially with plastidic localization, whereby the respective line disclose in column 7 the fine chemical glucose. For example, an increase of the glucose of at least 1 percent, particularly in a range of 137 to 173-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a yhl013c-protein, or if the activity of the polypeptide Yhl013c, preferably represented by SEQ ID NO. 14716, or a homolog or fragment thereof, or if the activity of a polypeptide encoded by a nucleic acid molecule comprising the nucleic acid SEQ ID NO. 14715, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 14715 or polypeptide SEQ ID NO. 14716, respectively, or a homolog or a fragment thereof, is increased or generated, or if the activity yhl013c-protein is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 18 to 38-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a 3-phosphoglycerate dehydrogenase, or if the activity of the polypeptide Yil074c, preferably represented by SEQ ID NO. 64564, or a homolog or fragment thereof, or if the activity of a polypeptide encoded by a nucleic acid molecule comprising the nucleic acid SEQ ID NO. 64563, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 64563 or polypeptide SEQ ID NO. 64564, respectively, or a homolog or a fragment thereof, is increased or generated, or if the activity 3-phosphoglycerate dehydrogenase is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 36 to 70-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a YIL083C-protein, or if the activity of the polypeptide Yil083c, preferably represented by SEQ ID NO. 149455, or a homolog or fragment thereof, or if the activity of a polypeptide encoded by a nucleic acid molecule comprising the nucleic acid SEQ ID NO. 149454, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 149454 or polypeptide SEQ ID NO. 149455, respectively, or a homolog or a fragment thereof, is increased or generated, or if the activity YIL083C-protein is increased or generated in a non-human organism, like a microorganism or a plant cell, plant or part thereof, especially with plastidic localization, whereby the respective line disclose in column 7 the fine chemical fructose. For example, an increase of the fructose of at least 1 percent, particularly in a range of 89 to 140-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a DnaJ-like chaperone, or if the activity of the polypeptide Yjl073w, preferably represented by SEQ ID NO. 64965, or a homolog or fragment thereof, or if the activity of a polypeptide encoded by a nucleic acid molecule comprising the nucleic acid SEQ ID NO. 64964, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 64964 or polypeptide SEQ ID NO. 64965, respectively, or a homolog or a fragment thereof, is increased or generated, or if the activity DnaJ-like chaperone is increased or generated in a non-human organism, like a microorganism or a plant cell, plant or part thereof, especially with plastidic localization, whereby the respective line disclose in column 7 the fine chemical myo-inositol. For example, an increase of the myo-inositol of at least 1 percent, particularly in a range of 25 to 34-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a YJL127W-A-protein, or if the activity of the polypeptide Yjl127w-a, preferably represented by SEQ ID NO. 97632, or a homolog or fragment thereof, or if the activity of a polypeptide encoded by a nucleic acid molecule comprising the nucleic acid SEQ ID NO. 97631, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 97631 or polypeptide SEQ ID NO. 97632, respectively, or a homolog or a fragment thereof, is increased or generated, or if the activity YJL127W-A-protein is increased or generated in a non-human organism, like a microorganism or a plant cell, plant or part thereof, especially with non-targeted localization, whereby the respective line disclose in column 7 the fine chemical 3,4,5-trihydroxypentanoic acid. For example, an increase of the 3,4,5-trihydroxypentanoic acid of at least 1 percent, particularly in a range of 39 to 102-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a inositol-3-phosphate synthase, or if the activity of the polypeptide Yjl153c, preferably represented by SEQ ID NO. 149633, or a homolog or fragment thereof, or if the activity of a polypeptide encoded by a nucleic acid molecule comprising the nucleic acid SEQ ID NO. 149632, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 149632 or polypeptide SEQ ID NO. 149633, respectively, or a homolog or a fragment thereof, is increased or generated, or if the activity inositol-3-phosphate synthase is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 51 to 79-percent is conferred as compared to a corresponding non-transformed wild type non-human organism.

Accordingly, in one embodiment, an increase of galactitol in a non-human organism, as compared to a corresponding non-transformed wild type non-human organism, is conferred in the process of the invention, if the activity of a polypeptide showing the activity of a inositol-3-phosphate synthase, or if the activity of the polypeptide Yjl153c, preferably represented by SEQ ID NO. 149633, or a homolog or fragment thereof, or if the activity of a polypeptide encoded by a nucleic acid molecule comprising the nucleic acid SEQ ID NO. 149632, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 149632 or polypeptide SEQ ID NO. 149633, respectively, or a homolog or a fragment thereof, is increased or generated, or if the activity inositol-3-phosphate synthase is increased or generated in a non-human organism, like a microorganism or a plant cell, plant or part thereof, especially with non-targeted localization, whereby the respective line disclose in column 7 the fine chemical galactitol. For example, an increase of the galactitol of at least 1 percent, particularly in a range of 96 to 292-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a inositol-3-phosphate synthase, or if the activity of the polypeptide Yjl153c, preferably represented by SEQ ID NO. 149633, or a homolog or fragment thereof, or if the activity of a polypeptide encoded by a nucleic acid molecule comprising the nucleic acid SEQ ID NO. 149632, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 149632 or polypeptide SEQ ID NO. 149633, respectively, or a homolog or a fragment thereof, is increased or generated, or if the activity inositol-3-phosphate synthase is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 24 to 70-percent is conferred as compared to a corresponding non-transformed wild type non-human organism.

Accordingly, in one embodiment, an increase of sorbitol in a non-human organism, as compared to a corresponding non-transformed wild type non-human organism, is conferred in the process of the invention, if the activity of a polypeptide showing the activity of a hexose transporter, or if the activity of the polypeptide Yjl219w, preferably represented by SEQ ID NO. 97636, or a homolog or fragment thereof, or if the activity of a polypeptide encoded by a nucleic acid molecule comprising the nucleic acid SEQ ID NO. 97635, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 97635 or polypeptide SEQ ID NO. 97636, respectively, or a homolog or a fragment thereof, is increased or generated, or if the activity hexose transporter is increased or generated in a non-human organism, like a microorganism or a plant cell, plant or part thereof, especially with non-targeted localization, whereby the respective line disclose in column 7 the fine chemical sorbitol. For example, an increase of the sorbitol of at least 1 percent, particularly in a range of 121 to 350-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a protein kinase, or if the activity of the polypeptide Yjr066w, preferably represented by SEQ ID NO. 149802, or a homolog or fragment thereof, or if the activity of a polypeptide encoded by a nucleic acid molecule comprising the nucleic acid SEQ ID NO. 149801, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 149801 or polypeptide SEQ ID NO. 149802, respectively, or a homolog or a fragment thereof, is increased or generated, or if the activity protein kinase is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 26 to 87-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a cystathionine gamma-synthase, or if the activity of the polypeptide Yjr130c, preferably represented by SEQ ID NO. 14844, or a homolog or fragment thereof, or if the activity of a polypeptide encoded by a nucleic acid molecule comprising the nucleic acid SEQ ID NO. 14843, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 14843 or polypeptide SEQ ID NO. 14844, respectively, or a homolog or a fragment thereof, is increased or generated, or if the activity cystathionine gamma-synthase is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 45 to 92-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a fructose biphosphate aldolase, or if the activity of the polypeptide Ykl060c, preferably represented by SEQ ID NO. 149869, or a homolog or fragment thereof, or if the activity of a polypeptide encoded by a nucleic acid molecule comprising the nucleic acid SEQ ID NO. 149868, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 149868 or polypeptide SEQ ID NO. 149869, respectively, or a homolog or a fragment thereof, is increased or generated, or if the activity fructose biphosphate aldolase is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 82 to 133-percent is conferred as compared to a corresponding non-transformed wild type non-human organism.

Accordingly, in one embodiment, an increase of sedoheptulose-7-phosphate in a non-human organism, as compared to a corresponding non-transformed wild type non-human organism, is conferred in the process of the invention, if the activity of a polypeptide showing the activity of a fructose biphosphate aldolase, or if the activity of the polypeptide Ykl060c, preferably represented by SEQ ID NO. 149869, or a homolog or fragment thereof, or if the activity of a polypeptide encoded by a nucleic acid molecule comprising the nucleic acid SEQ ID NO. 149868, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 149868 or polypeptide SEQ ID NO. 149869, respectively, or a homolog or a fragment thereof, is increased or generated, or if the activity fructose biphosphate aldolase is increased or generated in a non-human organism, like a microorganism or a plant cell, plant or part thereof, especially with non-targeted localization, whereby the respective line disclose in column 7 the fine chemical sedoheptulose-7-phosphate. For example, an increase of the sedoheptulose-7-phosphate of at least 1 percent, particularly in a range of 43 to 61-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a acyl carrier protein, or if the activity of the polypeptide Ykl192c, preferably represented by SEQ ID NO. 150064, or a homolog or fragment thereof, or if the activity of a polypeptide encoded by a nucleic acid molecule comprising the nucleic acid SEQ ID NO. 150063, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 150063 or polypeptide SEQ ID NO. 150064, respectively, or a homolog or a fragment thereof, is increased or generated, or if the activity acyl carrier protein is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 42 to 49-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a halotolerance protein, or if the activity of the polypeptide Ykr072c, preferably represented by SEQ ID NO. 130904, or a homolog or fragment thereof, or if the activity of a polypeptide encoded by a nucleic acid molecule comprising the nucleic acid SEQ ID NO. 130903, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 130903 or polypeptide SEQ ID NO. 130904, respectively, or a homolog or a fragment thereof, is increased or generated, or if the activity halotolerance protein is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 23 to 51-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a phosphoenolpyruvate carboxykinase, or if the activity of the polypeptide Ykr097w, preferably represented by SEQ ID NO. 98076, or a homolog or fragment thereof, or if the activity of a polypeptide encoded by a nucleic acid molecule comprising the nucleic acid SEQ ID NO. 98075, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 98075 or polypeptide SEQ ID NO. 98076, respectively, or a homolog or a fragment thereof, is increased or generated, or if the activity phosphoenolpyruvate carboxykinase is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 62 to 110-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a phosphoenolpyruvate carboxykinase, or if the activity of the polypeptide Ykr097w, preferably represented by SEQ ID NO. 98076, or a homolog or fragment thereof, or if the activity of a polypeptide encoded by a nucleic acid molecule comprising the nucleic acid SEQ ID NO. 98075, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 98075 or polypeptide SEQ ID NO. 98076, respectively, or a homolog or a fragment thereof, is increased or generated, or if the activity phosphoenolpyruvate carboxykinase is increased or generated in a non-human organism, like a microorganism or a plant cell, plant or part thereof, especially with plastidic localization, whereby the respective line disclose in column 7 the fine chemical glucose. For example, an increase of the glucose of at least 1 percent, particularly in a range of 70 to 93-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a vacuolar sorting protein, or if the activity of the polypeptide Ylr025w, preferably represented by SEQ ID NO. 150604, or a homolog or fragment thereof, or if the activity of a polypeptide encoded by a nucleic acid molecule comprising the nucleic acid SEQ ID NO. 150603, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 150603 or polypeptide SEQ ID NO. 150604, respectively, or a homolog or a fragment thereof, is increased or generated, or if the activity vacuolar sorting protein is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 31 to 31-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a thioredoxin, or if the activity of the polypeptide Ylr043c, preferably represented by SEQ ID NO. 91046, or a homolog or fragment thereof, or if the activity of a polypeptide encoded by a nucleic acid molecule comprising the nucleic acid SEQ ID NO. 91045, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 91045 or polypeptide SEQ ID NO. 91046, respectively, or a homolog or a fragment thereof, is increased or generated, or if the activity thioredoxin is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 19 to 78-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a hexose transporter, or if the activity of the polypeptide Ylr081w, preferably represented by SEQ ID NO. 150683, or a homolog or fragment thereof, or if the activity of a polypeptide encoded by a nucleic acid molecule comprising the nucleic acid SEQ ID NO. 150682, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 150682 or polypeptide SEQ ID NO. 150683, respectively, or a homolog or a fragment thereof, is increased or generated, or if the activity hexose transporter is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 68 to 128-percent is conferred as compared to a corresponding non-transformed wild type non-human organism.

Accordingly, in one embodiment, an increase of verbascose in a non-human organism, as compared to a corresponding non-transformed wild type non-human organism, is conferred in the process of the invention, if the activity of a polypeptide showing the activity of a purine biosynthesis protein, or if the activity of the polypeptide Ymr120c, preferably represented by SEQ ID NO. 91974, or a homolog or fragment thereof, or if the activity of a polypeptide encoded by a nucleic acid molecule comprising the nucleic acid SEQ ID NO. 91973, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 91973 or polypeptide SEQ ID NO. 91974, respectively, or a homolog or a fragment thereof, is increased or generated, or if the activity purine biosynthesis protein is increased or generated in a non-human organism, like a microorganism or a plant cell, plant or part thereof, especially with plastidic localization, whereby the respective line disclose in column 7 the fine chemical verbascose. For example, an increase of the verbascose of at least 1 percent, particularly in a range of 135 to 3405-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a ymr324c-protein, or if the activity of the polypeptide Ymr324c, preferably represented by SEQ ID NO. 130987, or a homolog or fragment thereof, or if the activity of a polypeptide encoded by a nucleic acid molecule comprising the nucleic acid SEQ ID NO. 130986, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 130986 or polypeptide SEQ ID NO. 130987, respectively, or a homolog or a fragment thereof, is increased or generated, or if the activity ymr324c-protein is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 34 to 58-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a molecular chaperone, or if the activity of the polypeptide Ynl064c, preferably represented by SEQ ID NO. 117496, or a homolog or fragment thereof, or if the activity of a polypeptide encoded by a nucleic acid molecule comprising the nucleic acid SEQ ID NO. 117495, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 117495 or polypeptide SEQ ID NO. 117496, respectively, or a homolog or a fragment thereof, is increased or generated, or if the activity molecular chaperone is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 28 to 50-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a molecular chaperone, or if the activity of the polypeptide Ynl064c, preferably represented by SEQ ID NO. 117496, or a homolog or fragment thereof, or if the activity of a polypeptide encoded by a nucleic acid molecule comprising the nucleic acid SEQ ID NO. 117495, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 117495 or polypeptide SEQ ID NO. 117496, respectively, or a homolog or a fragment thereof, is increased or generated, or if the activity molecular chaperone is increased or generated in a non-human organism, like a microorganism or a plant cell, plant or part thereof, especially with non-targeted localization, whereby the respective line disclose in column 7 the fine chemical sucrose. For example, an increase of the sucrose of at least 1 percent, particularly in a range of 25 to 31-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a sterol O-acyltransferase, or if the activity of the polypeptide Ynr019w, preferably represented by SEQ ID NO. 67647, or a homolog or fragment thereof, or if the activity of a polypeptide encoded by a nucleic acid molecule comprising the nucleic acid SEQ ID NO. 67646, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 67646 or polypeptide SEQ ID NO. 67647, respectively, or a homolog or a fragment thereof, is increased or generated, or if the activity sterol O-acyltransferase is increased or generated in a non-human organism, like a microorganism or a plant cell, plant or part thereof, especially with plastidic localization, whereby the respective line disclose in column 7 the fine chemical raffinose. For example, an increase of the raffinose of at least 1 percent, particularly in a range of 64 to 132-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a yol160w-protein, or if the activity of the polypeptide Yol160w, preferably represented by SEQ ID NO. 15180, or a homolog or fragment thereof, or if the activity of a polypeptide encoded by a nucleic acid molecule comprising the nucleic acid SEQ ID NO. 15179, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 15179 or polypeptide SEQ ID NO. 15180, respectively, or a homolog or a fragment thereof, is increased or generated, or if the activity yol160w-protein is increased or generated in a non-human organism, like a microorganism or a plant cell, plant or part thereof, especially with plastidic localization, whereby the respective line disclose in column 7 the fine chemical myo-inositol. For example, an increase of the myo-inositol of at least 1 percent, particularly in a range of 27 to 30-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a phosphohydrolase, or if the activity of the polypeptide Yor163w, preferably represented by SEQ ID NO. 151863, or a homolog or fragment thereof, or if the activity of a polypeptide encoded by a nucleic acid molecule comprising the nucleic acid SEQ ID NO. 151862, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 151862 or polypeptide SEQ ID NO. 151863, respectively, or a homolog or a fragment thereof, is increased or generated, or if the activity phosphohydrolase is increased or generated in a non-human organism, like a microorganism or a plant cell, plant or part thereof, especially with plastidic localization, whereby the respective line disclose in column 7 the fine chemical glucose. For example, an increase of the glucose of at least 1 percent, particularly in a range of 42 to 80-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a histone acetyltransferase, or if the activity of the polypeptide Yor244w, preferably represented by SEQ ID NO. 92541, or a homolog or fragment thereof, or if the activity of a polypeptide encoded by a nucleic acid molecule comprising the nucleic acid SEQ ID NO. 92540, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 92540 or polypeptide SEQ ID NO. 92541, respectively, or a homolog or a fragment thereof, is increased or generated, or if the activity histone acetyltransferase is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 31 to 53-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a subunit of COMPASS-protein complex, or if the activity of the polypeptide Ypl138c, preferably represented by SEQ ID NO. 151911, or a homolog or fragment thereof, or if the activity of a polypeptide encoded by a nucleic acid molecule comprising the nucleic acid SEQ ID NO. 151910, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 151910 or polypeptide SEQ ID NO. 151911, respectively, or a homolog or a fragment thereof, is increased or generated, or if the activity subunit of COMPASS-protein complex is increased or generated in a non-human organism, like a microorganism or a plant cell, plant or part thereof, especially with plastidic localization, whereby the respective line disclose in column 7 the fine chemical 3,4,5-trihydroxypentanoic acid. For example, an increase of the 3,4,5-trihydroxypentanoic acid of at least 1 percent, particularly in a range of 32 to 115-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a ypl144w-protein, or if the activity of the polypeptide Ypl144w, preferably represented by SEQ ID NO. 151928, or a homolog or fragment thereof, or if the activity of a polypeptide encoded by a nucleic acid molecule comprising the nucleic acid SEQ ID NO. 151927, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 151927 or polypeptide SEQ ID NO. 151928, respectively, or a homolog or a fragment thereof, is increased or generated, or if the activity ypl144w-protein is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 33 to 71-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a ypl144w-protein, or if the activity of the polypeptide Ypl144w, preferably represented by SEQ ID NO. 151928, or a homolog or fragment thereof, or if the activity of a polypeptide encoded by a nucleic acid molecule comprising the nucleic acid SEQ ID NO. 151927, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 151927 or polypeptide SEQ ID NO. 151928, respectively, or a homolog or a fragment thereof, is increased or generated, or if the activity ypl144w-protein is increased or generated in a non-human organism, like a microorganism or a plant cell, plant or part thereof, especially with plastidic localization, whereby the respective line disclose in column 7 the fine chemical 3,4,5-trihydroxypentanoic acid. For example, an increase of the 3,4,5-trihydroxypentanoic acid of at least 1 percent, particularly in a range of 36 to 66-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a ypr098c-protein, or if the activity of the polypeptide Ypr098c, preferably represented by SEQ ID NO. 151932, or a homolog or fragment thereof, or if the activity of a polypeptide encoded by a nucleic acid molecule comprising the nucleic acid SEQ ID NO. 151931, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 151931 or polypeptide SEQ ID NO. 151932, respectively, or a homolog or a fragment thereof, is increased or generated, or if the activity ypr098c-protein is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 47 to 110-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a ypr098c-protein, or if the activity of the polypeptide Ypr098c, preferably represented by SEQ ID NO. 151932, or a homolog or fragment thereof, or if the activity of a polypeptide encoded by a nucleic acid molecule comprising the nucleic acid SEQ ID NO. 151931, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 151931 or polypeptide SEQ ID NO. 151932, respectively, or a homolog or a fragment thereof, is increased or generated, or if the activity ypr098c-protein is increased or generated in a non-human organism, like a microorganism or a plant cell, plant or part thereof, especially with plastidic localization, whereby the respective line disclose in column 7 the fine chemical 3,4,5-trihydroxypentanoic acid. For example, an increase of the 3,4,5-trihydroxypentanoic acid of at least 1 percent, particularly in a range of 63 to 122-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a polyamine transporter, or if the activity of the polypeptide Ypr156c, preferably represented by SEQ ID NO. 151959, or a homolog or fragment thereof, or if the activity of a polypeptide encoded by a nucleic acid molecule comprising the nucleic acid SEQ ID NO. 151958, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 151958 or polypeptide SEQ ID NO. 151959, respectively, or a homolog or a fragment thereof, is increased or generated, or if the activity polyamine transporter is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 19 to 58-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a polyamine transporter, or if the activity of the polypeptide Ypr156c, preferably represented by SEQ ID NO. 151959, or a homolog or fragment thereof, or if the activity of a polypeptide encoded by a nucleic acid molecule comprising the nucleic acid SEQ ID NO. 151958, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 151958 or polypeptide SEQ ID NO. 151959, respectively, or a homolog or a fragment thereof, is increased or generated, or if the activity polyamine transporter is increased or generated in a non-human organism, like a microorganism or a plant cell, plant or part thereof, especially with plastidic localization, whereby the respective line disclose in column 7 the fine chemical myo-inositol. For example, an increase of the myo-inositol of at least 1 percent, particularly in a range of 27 to 92-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a Zm_{—}4842_BE510522-protein, or if the activity of the polypeptide Zm_{—}4842_BE510522, preferably represented by SEQ ID NO. 68414, or a homolog or fragment thereof, or if the activity of a polypeptide encoded by a nucleic acid molecule comprising the nucleic acid SEQ ID NO. 68413, preferably the coding region thereof, or a homolog or fragment thereof, e.g. derived from Zea mays, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 68413 or polypeptide SEQ ID NO. 68414, respectively, or a homolog or a fragment thereof, is increased or generated, or if the activity Zm_{—}4842_BE510522-protein is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 106 to 493-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a Zm_{—}4842_BE510522-protein, or if the activity of the polypeptide Zm_{—}4842_BE510522, preferably represented by SEQ ID NO. 68414, or a homolog or fragment thereof, or if the activity of a polypeptide encoded by a nucleic acid molecule comprising the nucleic acid SEQ ID NO. 68413, preferably the coding region thereof, or a homolog or fragment thereof, e.g. derived from Zea mays, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 68413 or polypeptide SEQ ID NO. 68414, respectively, or a homolog or a fragment thereof, is increased or generated, or if the activity Zm_{—}4842_BE510522-protein is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 68 to 257-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a acetyltransferase, or if the activity of the polypeptide 44849766_CANOLA, preferably represented by SEQ ID NO. 140627, or a homolog or fragment thereof, or if the activity of a polypeptide encoded by a nucleic acid molecule comprising the nucleic acid SEQ ID NO. 140626, preferably the coding region thereof, or a homolog or fragment thereof, e.g. derived from Brassica napus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 140626 or polypeptide SEQ ID NO. 140627, respectively, or a homolog or a fragment thereof, is increased or generated, or if the activity acetyltransferase is increased or generated in a non-human organism, like a microorganism or a plant cell, plant or part thereof, especially with plastidic localization, whereby the respective line disclose in column 7 the fine chemical myo-inositol. For example, an increase of the myo-inositol of at least 1 percent, particularly in a range of 36 to 104-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a 47266012-protein, or if the activity of the polypeptide 47266012_SOYBEAN, preferably represented by SEQ ID NO. 15188, or a homolog or fragment thereof, or if the activity of a polypeptide encoded by a nucleic acid molecule comprising the nucleic acid SEQ ID NO. 15187, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 15187 or polypeptide SEQ ID NO. 15188, respectively, or a homolog or a fragment thereof, is increased or generated, or if the activity 47266012-protein is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 78 to 200-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a 47266012-protein, or if the activity of the polypeptide 47266012_SOYBEAN, preferably represented by SEQ ID NO. 15188, or a homolog or fragment thereof, or if the activity of a polypeptide encoded by a nucleic acid molecule comprising the nucleic acid SEQ ID NO. 15187, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 15187 or polypeptide SEQ ID NO. 15188, respectively, or a homolog or a fragment thereof, is increased or generated, or if the activity 47266012-protein is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 60 to 140-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a 47266012-protein, or if the activity of the polypeptide 47266012_SOYBEAN, preferably represented by SEQ ID NO. 15188, or a homolog or fragment thereof, or if the activity of a polypeptide encoded by a nucleic acid molecule comprising the nucleic acid SEQ ID NO. 15187, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 15187 or polypeptide SEQ ID NO. 15188, respectively, or a homolog or a fragment thereof, is increased or generated, or if the activity 47266012-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 21 to 69-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a 47266012-protein, or if the activity of the polypeptide 47266012_SOYBEAN, preferably represented by SEQ ID NO. 15188, or a homolog or fragment thereof, or if the activity of a polypeptide encoded by a nucleic acid molecule comprising the nucleic acid SEQ ID NO. 15187, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 15187 or polypeptide SEQ ID NO. 15188, respectively, or a homolog or a fragment thereof, is increased or generated, or if the activity 47266012-protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 27 to 62-percent is conferred as compared to a corresponding non-transformed wild type non-human organism.

Accordingly, in one embodiment, an increase of sedoheptulose-7-phosphate in a non-human organism, as compared to a corresponding non-transformed wild type non-human organism, is conferred in the process of the invention, if the activity of a polypeptide showing the activity of a 47266012-protein, or if the activity of the polypeptide 47266012_SOYBEAN, preferably represented by SEQ ID NO. 15188, or a homolog or fragment thereof, or if the activity of a polypeptide encoded by a nucleic acid molecule comprising the nucleic acid SEQ ID NO. 15187, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 15187 or polypeptide SEQ ID NO. 15188, respectively, or a homolog or a fragment thereof, is increased or generated, or if the activity 47266012-protein is increased or generated in a non-human organism, like a microorganism or a plant cell, plant or part thereof, especially with non-targeted localization, whereby the respective line disclose in column 7 the fine chemical sedoheptulose7-phosphate. For example, an increase of the sedoheptulose-7-phosphate of at least 1 percent, particularly in a range of 48 to 72-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a 49747384_SOYBEAN-protein, or if the activity of the polypeptide 49747384_SOYBEAN, preferably represented by SEQ ID NO. 70, or a homolog or fragment thereof, or if the activity of a polypeptide encoded by a nucleic acid molecule comprising the nucleic acid SEQ ID NO. 69, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 69 or polypeptide SEQ ID NO. 70, respectively, or a homolog or a fragment thereof, is increased or generated, or if the activity 49747384_SOYBEAN-protein is increased or generated in a non-human organism, like a microorganism or a plant cell, plant or part thereof, especially with non-targeted localization, whereby the respective line disclose in column 7 the fine chemical glucose. For example, an increase of the glucose of at least 1 percent, particularly in a range of 101 to 120-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a 49747384_SOYBEAN-protein, or if the activity of the polypeptide 49747384_SOYBEAN, preferably represented by SEQ ID NO. 70, or a homolog or fragment thereof, or if the activity of a polypeptide encoded by a nucleic acid molecule comprising the nucleic acid SEQ ID NO. 69, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 69 or polypeptide SEQ ID NO. 70, respectively, or a homolog or a fragment thereof, is increased or generated, or if the activity 49747384_SOYBEAN-protein is increased or generated in a non-human organism, like a microorganism or a plant cell, plant or part thereof, especially with non-targeted localization, whereby the respective line disclose in column 7 the fine chemical glucose-1-phosphate. For example, an increase of the glucose-1-phosphate of at least 1 percent, particularly in a range of 27 to 72-percent is conferred as compared to a corresponding non-transformed wild type non-human organism.

Accordingly, in one embodiment, an increase of myo-inositol in a non-human organism, as compared to a corresponding non-transformed wild type non-human organism, is conferred in the process of the invention, if the activity of a polypeptide showing the activity of a 49747384_SOYBEAN-protein, or if the activity of the polypeptide 49747384_SOYBEAN, preferably represented by SEQ ID NO. 70, or a homolog or fragment thereof, or if the activity of a polypeptide encoded by a nucleic acid molecule comprising the nucleic acid SEQ ID NO. 69, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 69 or polypeptide SEQ ID NO. 70, respectively, or a homolog or a fragment thereof, is increased or generated, or if the activity 49747384_SOYBEAN-protein is increased or generated in a non-human organism, like a microorganism or a plant cell, plant or part thereof, especially with non-targeted localization, whereby the respective line disclose in column 7 the fine chemical myo-inositol. For example, an increase of the myo-inositol of at least 1 percent, particularly in a range of 33 to 71-percent is conferred as compared to a corresponding non-transformed wild type non-human organism.

Accordingly, in one embodiment, an increase of raffinose in a non-human organism, as compared to a corresponding non-transformed wild type non-human organism, is conferred in the process of the invention, if the activity of a polypeptide showing the activity of a 49747384_SOYBEAN-protein, or if the activity of the polypeptide 49747384_SOYBEAN, preferably represented by SEQ ID NO. 70, or a homolog or fragment thereof, or if the activity of a polypeptide encoded by a nucleic acid molecule comprising the nucleic acid SEQ ID NO. 69, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 69 or polypeptide SEQ ID NO. 70, respectively, or a homolog or a fragment thereof, is increased or generated, or if the activity 49747384_SOYBEAN-protein is increased or generated in a non-human organism, like a microorganism or a plant cell, plant or part thereof, especially with non-targeted localization, whereby the respective line disclose in column 7 the fine chemical raffinose. For example, an increase of the raffinose of at least 1 percent, particularly in a range of 64 to 155-percent is conferred as compared to a corresponding non-transformed wild type non-human organism.

Accordingly, in one embodiment, an increase of sedoheptulose-7-phosphate in a non-human organism, as compared to a corresponding non-transformed wild type non-human organism, is conferred in the process of the invention, if the activity of a polypeptide showing the activity of a 49747384_SOYBEAN-protein, or if the activity of the polypeptide 49747384_SOYBEAN, preferably represented by SEQ ID NO. 70, or a homolog or fragment thereof, or if the activity of a polypeptide encoded by a nucleic acid molecule comprising the nucleic acid SEQ ID NO. 69, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 69 or polypeptide SEQ ID NO. 70, respectively, or a homolog or a fragment thereof, is increased or generated, or if the activity 49747384_SOYBEAN-protein is increased or generated in a non-human organism, like a microorganism or a plant cell, plant or part thereof, especially with non-targeted localization, whereby the respective line disclose in column 7 the fine chemical sedoheptulose-7-phosphate. For example, an increase of the sedoheptulose-7-phosphate of at least 1-percent, particularly in a range of 46 to 85-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a glucose-6-phosphate 1-dehydrogenase, or if the activity of the polypeptide 59582753_SOYBEAN, preferably represented by SEQ ID NO. 16884, or a homolog or fragment thereof, or if the activity of a polypeptide encoded by a nucleic acid molecule comprising the nucleic acid SEQ ID NO. 16883, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 16883 or polypeptide SEQ ID NO. 16884, respectively, or a homolog or a fragment thereof, is increased or generated, or if the activity glucose-6-phosphate 1-dehydrogenase is increased or generated in a non-human organism, like a microorganism or a plant cell, plant or part thereof, especially with plastidic localization, whereby the respective line disclose in column 7 the fine chemical fructose. For example, an increase of the fructose of at least 1 percent, particularly in a range of 77 to 404-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a glucose-6-phosphate 1-dehydrogenase, or if the activity of the polypeptide 59582753_SOYBEAN, preferably represented by SEQ ID NO. 16884, or a homolog or fragment thereof, or if the activity of a polypeptide encoded by a nucleic acid molecule comprising the nucleic acid SEQ ID NO. 16883, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 16883 or polypeptide SEQ ID NO. 16884, respectively, or a homolog or a fragment thereof, is increased or generated, or if the activity glucose-6-phosphate 1-dehydrogenase is increased or generated in a non-human organism, like a microorganism or a plant cell, plant or part thereof, especially with plastidic localization, whereby the respective line disclose in column 7 the fine chemical glucose. For example, an increase of the glucose of at least 1 percent, particularly in a range of 98 to 367-percent is conferred as compared to a corresponding non-transformed wild type non-human organism.

Accordingly, in one embodiment, an increase of glucose-1-phosphate in a non-human organism, as compared to a corresponding non-transformed wild type non-human organism, is conferred in the process of the invention, if the activity of a polypeptide showing the activity of a glucose-6-phosphate 1-dehydrogenase, or if the activity of the polypeptide 59582753_SOYBEAN, preferably represented by SEQ ID NO. 16884, or a homolog or fragment thereof, or if the activity of a polypeptide encoded by a nucleic acid molecule comprising the nucleic acid SEQ ID NO. 16883, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 16883 or polypeptide SEQ ID NO. 16884, respectively, or a homolog or a fragment thereof, is increased or generated, or if the activity glucose-6-phosphate 1-dehydrogenase is increased or generated in a non-human organism, like a microorganism or a plant cell, plant or part thereof, especially with plastidic localization, whereby the respective line disclose in column 7 the fine chemical glucose-1-phosphate. For example, an increase of the glucose-1-phosphate of at least 1 percent, particularly in a range of 32 to 101-percent is conferred as compared to a corresponding non-transformed wild type non-human organism.

Accordingly, in one embodiment, an increase of sedoheptulose-7-phosphate in a non-human organism, as compared to a corresponding non-transformed wild type non-human organism, is conferred in the process of the invention, if the activity of a polypeptide showing the activity of a glucose-6-phosphate 1-dehydrogenase, or if the activity of the polypeptide 59582753_SOYBEAN, preferably represented by SEQ ID NO. 16884, or a homolog or fragment thereof, or if the activity of a polypeptide encoded by a nucleic acid molecule comprising the nucleic acid SEQ ID NO. 16883, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 16883 or polypeptide SEQ ID NO. 16884, respectively, or a homolog or a fragment thereof, is increased or generated, or if the activity glucose-6-phosphate 1-dehydrogenase is increased or generated in a non-human organism, like a microorganism or a plant cell, plant or part thereof, especially with plastidic localization, whereby the respective line disclose in column 7 the fine chemical sedoheptulose-7-phosphate. For example, an increase of the sedoheptulose-7-phosphate of at least 1 percent, particularly in a range of 56 to 350-percent is conferred as compared to a corresponding non-transformed wild type non-human organism.

Accordingly, in one embodiment, an increase of sucrose in a non-human organism, as compared to a corresponding non-transformed wild type non-human organism, is conferred in the process of the invention, if the activity of a polypeptide showing the activity of a glucose-6-phosphate 1-dehydrogenase, or if the activity of the polypeptide 59582753_SOYBEAN, preferably represented by SEQ ID NO. 16884, or a homolog or fragment thereof, or if the activity of a polypeptide encoded by a nucleic acid molecule comprising the nucleic acid SEQ ID NO. 16883, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 16883 or polypeptide SEQ ID NO. 16884, respectively, or a homolog or a fragment thereof, is increased or generated, or if the activity glucose-6-phosphate 1-dehydrogenase is increased or generated in a non-human organism, like a microorganism or a plant cell, plant or part thereof, especially with plastidic localization, whereby the respective line disclose in column 7 the fine chemical sucrose. For example, an increase of the sucrose of at least 1 percent, particularly in a range of 30 to 79-percent is conferred as compared to a corresponding non-transformed wild type non-human organism.

Accordingly, in one embodiment, an increase of 3,4,5-trihydroxypentanoic acid in a non-human organism, as compared to a corresponding non-transformed wild type non-human organism, is conferred in the process of the invention, if the activity of a polypeptide showing the activity of a glucose-6-phosphate 1-dehydrogenase, or if the activity of the polypeptide 59582753_SOYBEAN, preferably represented by SEQ ID NO. 16884, or a homolog or fragment thereof, or if the activity of a polypeptide encoded by a nucleic acid molecule comprising the nucleic acid SEQ ID NO. 16883, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 16883 or polypeptide SEQ ID NO. 16884, respectively, or a homolog or a fragment thereof, is increased or generated, or if the activity glucose-6-phosphate 1-dehydrogenase is increased or generated in a non-human organism, like a microorganism or a plant cell, plant or part thereof, especially with plastidic localization, whereby the respective line disclose in column 7 the fine chemical 3,4,5-trihydroxypentanoic acid. For example, an increase of the 3,4,5-trihydroxypentanoic acid of at least 1 percent, particularly in a range of 37 to 139-percent is conferred as compared to a corresponding non-transformed wild type non-human organism.

Accordingly, in one embodiment, an increase of fructose in a non-human organism, as compared to a corresponding non-transformed wild type non-human organism, is conferred in the process of the invention, if the activity of a polypeptide showing the activity of a DNA replication complex GINS protein, or if the activity of the polypeptide YJL072C, preferably represented by SEQ ID NO. 149529, or a homolog or fragment thereof, or if the activity of a polypeptide encoded by a nucleic acid molecule comprising the nucleic acid SEQ ID NO. 149528, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 149528 or polypeptide SEQ ID NO. 149529, respectively, or a homolog or a fragment thereof, is increased or generated, or if the activity DNA replication complex GINS protein is increased or generated in a non-human organism, like a microorganism or a plant cell, plant or part thereof, especially with non-targeted localization, whereby the respective line disclose in column 7 the fine chemical fructose. For example, an increase of the fructose of at least 1 percent, particularly in a range of 1 to 418-percent is conferred as compared to a corresponding non-transformed wild type non-human organism.

Accordingly, in one embodiment, an increase of glucose in a non-human organism, as compared to a corresponding non-transformed wild type non-human organism, is conferred in the process of the invention, if the activity of a polypeptide showing the activity of a acetyl CoA synthetase, or if the activity of the polypeptide YLR153C, preferably represented by SEQ ID NO. 150961, or a homolog or fragment thereof, or if the activity of a polypeptide encoded by a nucleic acid molecule comprising the nucleic acid SEQ ID NO. 150960, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 17, column 5 or 8 in the respective same line as the nucleic acid molecule SEQ ID NO. 150960 or polypeptide SEQ ID NO. 150961, respectively, or a homolog or a fragment thereof, is increased or generated, or if the activity acetyl CoA synthetase is increased or generated in a non-human organism, like a microorganism or a plant cell, plant or part thereof, especially with plastidic localization, whereby the respective line disclose in column 7 the fine chemical glucose. For example, an increase of the glucose of at least 1 percent, particularly in a range of 12 to 372-percent is conferred as compared to a corresponding non-transformed wild type non-human organism.

[0096.1.7.17] to for the disclosure of these paragraphs see [0096.1.7.7] to [0103.1.7.7] above

In this paragraph “A” means “Goffeau et al., Science 274 (5287), 546 (1996)”, “B” means “Blattner et al., Science 277 (5331), 1453 (1997)”, “C” means “Nakamura et al., Nucleic Acids Res. 28(1), 72 (2000)”, “D” means “deposition of the sequences to the EMBL/GenBank/DDBJ databases under “Sequencing of the draft genome assembly of Azotobacter vinelandii AvOP”. in June 2005 by Copeland et al.”, “E” means “Henne et al., Nat. Biotechnol. 22 (5), 547 (2004)”, “F” means “Kaul et al., Nature 408 (6814), 796 (2000)”, and “G” means “Matsumoto et al., Nature 436 (7052), 793 (2005)”.

The nucleic acid sequence of Atgols2 from Arabidopsis thaliana, e.g. as shown in the respective line in column 5 of Table I, application no. 17, has been published in F. And the activity of the gene product thereof is the activity of galactinol synthase.

Accordingly, in one embodiment, the process of the present invention for producing raffinose in a non-human organism, like a microorganism or a plant or a part thereof, comprises increasing or generating the activity of a gene product with the activity of a gene product conferring the activity of “galactinol synthase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said Atgols2, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Atgols2, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Atgols2, or a functional equivalent or a homolog thereof as depicted in column 8 of Table II, application no. 17, preferably a homolog or functional equivalent as depicted in column 8 of Table II B, application no. 17, and being depicted in the same respective line as said Atgols2, and preferably the activity is increased non-targeted, whereby the respective line disclose in column 7 the fine chemical raffinose.

Accordingly, in one embodiment, the molecule which activity is to be increased in the process of the invention is the gene product with an activity as a “galactinol synthase”, preferably it is the molecule of section (a) or (b) of this paragraph.

In particular, it was observed that in plants, especially in Arabidopsis thaliana, increasing or generating the activity of a gene product non-targeted with the activity of a “galactinol synthase”, preferably being encoded by a gene comprising the nucleic acid sequence SEQ ID NO. 142992, preferably the coding region thereof, conferred the production of or the increase in raffinose compared with the wild type control.

The nucleic acid sequence of At1g07430 from Arabidopsis thaliana, e.g. as shown in the respective line in column 5 of Table I, application no. 17, has been published in F. And the activity of the gene product thereof is the activity of protein phosphatase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said At1g07430, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At1g07430, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At1g07430, or a functional equivalent or a homolog thereof as depicted in column 8 of Table II, application no. 17, preferably a homolog or functional equivalent as depicted in column 8 of Table II B, application no. 17, and being depicted in the same respective line as said At1g07430, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 17451, preferably the coding region thereof, conferred the production of or the increase in glucose-1-phosphate compared with the wild type control.

The nucleic acid sequence of At1g07430 from Arabidopsis thaliana, e.g. as shown in the respective line in column 5 of Table I, application no. 17, has been published in F. And the activity of the gene product thereof is the activity of protein phosphatase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said At1g07430, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At1g07430, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At1g07430, or a functional equivalent or a homolog thereof as depicted in column 8 of Table II, application no. 17, preferably a homolog or functional equivalent as depicted in column 8 of Table II B, application no. 17, and being depicted in the same respective line as said At1g07430, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 17451, preferably the coding region thereof, conferred the production of or the increase in myo-inositol compared with the wild type control.

The nucleic acid sequence of At1g19350 from Arabidopsis thaliana, e.g. as shown in the respective line in column 5 of Table I, application no. 17, has been published in F. And the activity of the gene product thereof is the activity of At1g19350-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “At1g19350-protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said At1g19350, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At1g19350, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At1g19350, or a functional equivalent or a homolog thereof as depicted in column 8 of Table II, application no. 17, preferably a homolog or functional equivalent as depicted in column 8 of Table II B, application no. 17, and being depicted in the same respective line as said At1g19350, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “At1g19350-protein”, preferably it is the molecule of section (a) or (b) of this paragraph.

In particular, it was observed that in plants, especially in Arabidopsis thaliana, increasing or generating the activity of a gene product non-targeted with the activity of a “At1g19350-protein”, preferably being encoded by a gene comprising the nucleic acid sequence SEQ ID NO. 140771, preferably the coding region thereof, conferred the production of or the increase in glucose-1-phosphate compared with the wild type control.

The nucleic acid sequence of At1g19350 from Arabidopsis thaliana, e.g. as shown in the respective line in column 5 of Table I, application no. 17, has been published in F. And the activity of the gene product thereof is the activity of At1g19350-protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “At1g19350-protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said At1g19350, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At1g19350, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At1g19350, or a functional equivalent or a homolog thereof as depicted in column 8 of Table II, application no. 17, preferably a homolog or functional equivalent as depicted in column 8 of Table II B, application no. 17, and being depicted in the same respective line as said At1g19350, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “At1g19350-protein”, preferably it is the molecule of section (a) or (b) of this paragraph.

In particular, it was observed that in plants, especially in Arabidopsis thaliana, increasing or generating the activity of a gene product non-targeted with the activity of a “At1g19350-protein”, preferably being encoded by a gene comprising the nucleic acid sequence SEQ ID NO. 140771, preferably the coding region thereof, conferred the production of or the increase in 3,4,5-trihydroxypentanoic acid compared with the wild type control.

The nucleic acid sequence of At1g19670 from Arabidopsis thaliana, e.g. as shown in the respective line in column 5 of Table I, application no. 17, has been published in F. And the activity of the gene product thereof is the activity of chlorophyllase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “chlorophyllase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said At1g19670, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At1g19670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At1g19670, or a functional equivalent or a homolog thereof as depicted in column 8 of Table II, application no. 17, preferably a homolog or functional equivalent as depicted in column 8 of Table II B, application no. 17, and being depicted in the same respective line as said At1g19670, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “chlorophyllase”, preferably it is the molecule of section (a) or (b) of this paragraph.

In particular, it was observed that in plants, especially in Arabidopsis thaliana, increasing or generating the activity of a gene product non-targeted with the activity of a “chlorophyllase”, preferably being encoded by a gene comprising the nucleic acid sequence SEQ ID NO. 113651, preferably the coding region thereof, conferred the production of or the increase in myo-inositol compared with the wild type control.

The nucleic acid sequence of At1g26830 from Arabidopsis thaliana, e.g. as shown in the respective line in column 5 of Table I, application no. 17, has been published in F. And the activity of the gene product thereof is the activity of cullin.

Accordingly, in one embodiment, the process of the present invention for producing fructose in a non-human organism, like a microorganism or a plant or a part thereof, comprises increasing or generating the activity of a gene product with the activity of a gene product conferring the activity of “cullin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said At1g26830, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At1g26830, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At1g26830, or a functional equivalent or a homolog thereof as depicted in column 8 of Table II, application no. 17, preferably a homolog or functional equivalent as depicted in column 8 of Table II B, application no. 17, and being depicted in the same respective line as said At1g26830, and preferably the activity is increased non-targeted, whereby the respective line disclose in column 7 the fine chemical fructose.

Accordingly, in one embodiment, the molecule which activity is to be increased in the process of the invention is the gene product with an activity as a “cullin”, preferably it is the molecule of section (a) or (b) of this paragraph.

In particular, it was observed that in plants, especially in Arabidopsis thaliana, increasing or generating the activity of a gene product non-targeted with the activity of a “cullin”, preferably being encoded by a gene comprising the nucleic acid sequence SEQ ID NO. 17701, preferably the coding region thereof, conferred the production of or the increase in fructose compared with the wild type control.

The nucleic acid sequence of At1g36730 from Arabidopsis thaliana, e.g. as shown in the respective line in column 5 of Table I, application no. 17, has been published in F. And the activity of the gene product thereof is the activity of eukaryotic translation initiation factor 5.

Accordingly, in one embodiment, the process of the present invention for producing fructose in a non-human organism, like a microorganism or a plant or a part thereof, comprises increasing or generating the activity of a gene product with the activity of a gene product conferring the activity of “eukaryotic translation initiation factor 5”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said At1g36730, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At1g36730, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At1g36730, or a functional equivalent or a homolog thereof as depicted in column 8 of Table II, application no. 17, preferably a homolog or functional equivalent as depicted in column 8 of Table II B, application no. 17, and being depicted in the same respective line as said At1g36730, and preferably the activity is increased non-targeted, whereby the respective line disclose in column 7 the fine chemical fructose.

Accordingly, in one embodiment, the molecule which activity is to be increased in the process of the invention is the gene product with an activity as a “eukaryotic translation initiation factor 5”, preferably it is the molecule of section (a) or (b) of this paragraph.

In particular, it was observed that in plants, especially in Arabidopsis thaliana, increasing or generating the activity of a gene product non-targeted with the activity of a “eukaryotic translation initiation factor 5”, preferably being encoded by a gene comprising the nucleic acid sequence SEQ ID NO. 17968, preferably the coding region thereof, conferred the production of or the increase in fructose compared with the wild type control.

The nucleic acid sequence of At1g36730 from Arabidopsis thaliana, e.g. as shown in the respective line in column 5 of Table I, application no. 17, has been published in F. And the activity of the gene product thereof is the activity of eukaryotic translation initiation factor 5.

Accordingly, in one embodiment, the process of the present invention for producing glucose in a non-human organism, like a microorganism or a plant or a part thereof, comprises increasing or generating the activity of a gene product with the activity of a gene product conferring the activity of “eukaryotic translation initiation factor 5”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said At1g36730, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At1g36730, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At1g36730, or a functional equivalent or a homolog thereof as depicted in column 8 of Table II, application no. 17, preferably a homolog or functional equivalent as depicted in column 8 of Table II B, application no. 17, and being depicted in the same respective line as said At1g36730, and preferably the activity is increased non-targeted, whereby the respective line disclose in column 7 the fine chemical glucose.

Accordingly, in one embodiment, the molecule which activity is to be increased in the process of the invention is the gene product with an activity as a “eukaryotic translation initiation factor 5”, preferably it is the molecule of section (a) or (b) of this paragraph.

In particular, it was observed that in plants, especially in Arabidopsis thaliana, increasing or generating the activity of a gene product non-targeted with the activity of a “eukaryotic translation initiation factor 5”, preferably being encoded by a gene comprising the nucleic acid sequence SEQ ID NO. 17968, preferably the coding region thereof, conferred the production of or the increase in glucose compared with the wild type control.

The nucleic acid sequence of At1g36730 from Arabidopsis thaliana, e.g. as shown in the respective line in column 5 of Table I, application no. 17, has been published in F. And the activity of the gene product thereof is the activity of eukaryotic translation initiation factor 5.

Accordingly, in one embodiment, the process of the present invention for producing raffinose in a non-human organism, like a microorganism or a plant or a part thereof, comprises increasing or generating the activity of a gene product with the activity of a gene product conferring the activity of “eukaryotic translation initiation factor 5”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said At1g36730, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At1g36730, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At1g36730, or a functional equivalent or a homolog thereof as depicted in column 8 of Table II, application no. 17, preferably a homolog or functional equivalent as depicted in column 8 of Table II B, application no. 17, and being depicted in the same respective line as said At1g36730, and preferably the activity is increased non-targeted, whereby the respective line disclose in column 7 the fine chemical raffinose.

Accordingly, in one embodiment, the molecule which activity is to be increased in the process of the invention is the gene product with an activity as a “eukaryotic translation initiation factor 5”, preferably it is the molecule of section (a) or (b) of this paragraph.

In particular, it was observed that in plants, especially in Arabidopsis thaliana, increasing or generating the activity of a gene product non-targeted with the activity of a “eukaryotic translation initiation factor 5”, preferably being encoded by a gene comprising the nucleic acid sequence SEQ ID NO. 17968, preferably the coding region thereof, conferred the production of or the increase in raffinose compared with the wild type control.

The nucleic acid sequence of At1g43850 from Arabidopsis thaliana, e.g. as shown in the respective line in column 5 of Table I, application no. 17, has been published in F. And the activity of the gene product thereof is the activity of transcriptional regulator.

Accordingly, in one embodiment, the process of the present invention for producing sucrose in a non-human organism, like a microorganism or a plant or a part thereof, comprises increasing or generating the activity of a gene product with the activity of a gene product conferring the activity of “transcriptional regulator”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said At1g43850, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At1g43850, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At1g43850, or a functional equivalent or a homolog thereof as depicted in column 8 of Table II, application no. 17, preferably a homolog or functional equivalent as depicted in column 8 of Table II B, application no. 17, and being depicted in the same respective line as said At1g43850, and preferably the activity is increased non-targeted, whereby the respective line disclose in column 7 the fine chemical sucrose.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 18070, preferably the coding region thereof, conferred the production of or the increase in sucrose compared with the wild type control.

The nucleic acid sequence of At1g60470 from Arabidopsis thaliana, e.g. as shown in the respective line in column 5 of Table I, application no. 17, has been published in F. And the activity of the gene product thereof is the activity of galactinol synthase.

Accordingly, in one embodiment, the process of the present invention for producing raffinose in a non-human organism, like a microorganism or a plant or a part thereof, comprises increasing or generating the activity of a gene product with the activity of a gene product conferring the activity of “galactinol synthase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said At1g60470, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At1g60470, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At1g60470, or a functional equivalent or a homolog thereof as depicted in column 8 of Table II, application no. 17, preferably a homolog or functional equivalent as depicted in column 8 of Table II B, application no. 17, and being depicted in the same respective line as said At1g60470, and preferably the activity is increased non-targeted, whereby the respective line disclose in column 7 the fine chemical raffinose.

Accordingly, in one embodiment, the molecule which activity is to be increased in the process of the invention is the gene product with an activity as a “galactinol synthase”, preferably it is the molecule of section (a) or (b) of this paragraph.

In particular, it was observed that in plants, especially in Arabidopsis thaliana, increasing or generating the activity of a gene product non-targeted with the activity of a “galactinol synthase”, preferably being encoded by a gene comprising the nucleic acid sequence SEQ ID NO. 140841, preferably the coding region thereof, conferred the production of or the increase in raffinose compared with the wild type control.

The nucleic acid sequence of At1g72770 from Arabidopsis thaliana, e.g. as shown in the respective line in column 5 of Table I, application no. 17, has been published in F. And the activity of the gene product thereof is the activity of protein phosphatase.

Accordingly, in one embodiment, the process of the present invention for producing glucose in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said At1g72770, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At1g72770, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At1g72770, or a functional equivalent or a homolog thereof as depicted in column 8 of Table II, application no. 17, preferably a homolog or functional equivalent as depicted in column 8 of Table II B, application no. 17, and being depicted in the same respective line as said At1g72770, and preferably the activity is increased non-targeted, whereby the respective line disclose in column 7 the fine chemical glucose.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19419, preferably the coding region thereof, conferred the production of or the increase in glucose compared with the wild type control.

The nucleic acid sequence of At1g72770 from Arabidopsis thaliana, e.g. as shown in the respective line in column 5 of Table I, application no. 17, has been published in F. And the activity of the gene product thereof is the activity of protein phosphatase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said At1g72770, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At1g72770, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At1g72770, or a functional equivalent or a homolog thereof as depicted in column 8 of Table II, application no. 17, preferably a homolog or functional equivalent as depicted in column 8 of Table II B, application no. 17, and being depicted in the same respective line as said At1g72770, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19419, preferably the coding region thereof, conferred the production of or the increase in glucose-1-phosphate compared with the wild type control.

The nucleic acid sequence of At1g72770 from Arabidopsis thaliana, e.g. as shown in the respective line in column 5 of Table I, application no. 17, has been published in F. And the activity of the gene product thereof is the activity of protein phosphatase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said At1g72770, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At1g72770, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At1g72770, or a functional equivalent or a homolog thereof as depicted in column 8 of Table II, application no. 17, preferably a homolog or functional equivalent as depicted in column 8 of Table II B, application no. 17, and being depicted in the same respective line as said At1g72770, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19419, preferably the coding region thereof, conferred the production of or the increase in myo-inositol compared with the wild type control.

The nucleic acid sequence of At1g72770 from Arabidopsis thaliana, e.g. as shown in the respective line in column 5 of Table I, application no. 17, has been published in F. And the activity of the gene product thereof is the activity of protein phosphatase.

Accordingly, in one embodiment, the process of the present invention for producing raffinose in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said At1g72770, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At1g72770, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At1g72770, or a functional equivalent or a homolog thereof as depicted in column 8 of Table II, application no. 17, preferably a homolog or functional equivalent as depicted in column 8 of Table II B, application no. 17, and being depicted in the same respective line as said At1g72770, and preferably the activity is increased non-targeted, whereby the respective line disclose in column 7 the fine chemical raffinose.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19419, preferably the coding region thereof, conferred the production of or the increase in raffinose compared with the wild type control.

The nucleic acid sequence of At1g72770 from Arabidopsis thaliana, e.g. as shown in the respective line in column 5 of Table I, application no. 17, has been published in F. And the activity of the gene product thereof is the activity of protein phosphatase.

Accordingly, in one embodiment, the process of the present invention for producing sucrose in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said At1g72770, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At1g72770, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At1g72770, or a functional equivalent or a homolog thereof as depicted in column 8 of Table II, application no. 17, preferably a homolog or functional equivalent as depicted in column 8 of Table II B, application no. 17, and being depicted in the same respective line as said At1g72770, and preferably the activity is increased non-targeted, whereby the respective line disclose in column 7 the fine chemical sucrose.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19419, preferably the coding region thereof, conferred the production of or the increase in sucrose compared with the wild type control.

The nucleic acid sequence of At1g78200 from Arabidopsis thaliana, e.g. as shown in the respective line in column 5 of Table I, application no. 17, has been published in F. And the activity of the gene product thereof is the activity of protein phosphatase.

Accordingly, in one embodiment, the process of the present invention for producing saccharic acid in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical saccharic acid), application no. 17, preferably the coding region thereof, or a homolog or a fragment thereof, and being depicted in the same respective line as said At1g78200, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At1g78200, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At1g78200, or a functional equivalent or a homolog thereof as depicted in column 8 of Table II, application no. 17, preferably a homolog or functional equivalent as depicted in column 8 of Table II B, application no. 17, and being depicted in the same respective line as said At1g78200, and preferably the activity is increased non-targeted, whereby the respective line disclose in column 7 the fine chemical saccharic acid.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 141013, preferably the coding region thereof, conferred the production of or the increase in saccharic acid compared with the wild type control.

The nucleic acid sequence of At2g17560 from Arabidopsis thaliana, e.g. as shown in the respective line in column 5 of Table I, application no. 17, has been published in F. And the activity of the gene product thereof is the activity of transcription factor.

Accordingly, in one embodiment, the process of the present invention for producing fructose in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said At2g17560, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At2g17560, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At2g17560, or a functional equivalent or a homolog thereof as depicted in column 8 of Table II, application no. 17, preferably a homolog or functional equivalent as depicted in column 8 of Table II B, application no. 17, and being depicted in the same respective line as said At2g17560, and preferably the activity is increased non-targeted, whereby the respective line disclose in column 7 the fine chemical fructose.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 19502, preferably the coding region thereof, conferred the production of or the increase in fructose compared with the wild type control.

The nucleic acid sequence of At2g17560 from Arabidopsis thaliana, e.g. as shown in the respective line in column 5 of Table I, application no. 17, has been published in F. And the activity of the gene product thereof is the activity of transcription factor.

Accordingly, in one embodiment, the process of the present invention for producing glucose in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said At2g17560, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At2g17560, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At2g17560, or a functional equivalent or a homolog thereof as depicted in column 8 of Table II, application no. 17, preferably a homolog or functional equivalent as depicted in column 8 of Table II B, application no. 17, and being depicted in the same respective line as said At2g17560, and preferably the activity is increased non-targeted, whereby the respective line disclose in column 7 the fine chemical glucose.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 19502, preferably the coding region thereof, conferred the production of or the increase in glucose compared with the wild type control.

The nucleic acid sequence of At2g17560 from Arabidopsis thaliana, e.g. as shown in the respective line in column 5 of Table I, application no. 17, has been published in F. And the activity of the gene product thereof is the activity of transcription factor.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said At2g17560, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At2g17560, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At2g17560, or a functional equivalent or a homolog thereof as depicted in column 8 of Table II, application no. 17, preferably a homolog or functional equivalent as depicted in column 8 of Table II B, application no. 17, and being depicted in the same respective line as said At2g17560, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 19502, preferably the coding region thereof, conferred the production of or the increase in glucose-1-phosphate compared with the wild type control.

The nucleic acid sequence of At2g25070 from Arabidopsis thaliana, e.g. as shown in the respective line in column 5 of Table I, application no. 17, has been published in F. And the activity of the gene product thereof is the activity of protein phosphatase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said At2g25070, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At2g25070, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At2g25070, or a functional equivalent or a homolog thereof as depicted in column 8 of Table II, application no. 17, preferably a homolog or functional equivalent as depicted in column 8 of Table II B, application no. 17, and being depicted in the same respective line as said At2g25070, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 1298, preferably the coding region thereof, conferred the production of or the increase in glucose-1-phosphate compared with the wild type control.

The nucleic acid sequence of At2g30540 from Arabidopsis thaliana, e.g. as shown in the respective line in column 5 of Table I, application no. 17, has been published in F. And the activity of the gene product thereof is the activity of glutaredoxin.

Accordingly, in one embodiment, the process of the present invention for producing saccharic acid in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical saccharic acid), application no. 17, preferably the coding region thereof, or a homolog or a fragment thereof, and being depicted in the same respective line as said At2g30540, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At2g30540, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At2g30540, or a functional equivalent or a homolog thereof as depicted in column 8 of Table II, application no. 17, preferably a homolog or functional equivalent as depicted in column 8 of Table II B, application no. 17, and being depicted in the same respective line as said At2g30540, and preferably the activity is increased non-targeted, whereby the respective line disclose in column 7 the fine chemical saccharic acid.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 20346, preferably the coding region thereof, conferred the production of or the increase in saccharic acid compared with the wild type control.

The nucleic acid sequence of At2g34180 from Arabidopsis thaliana, e.g. as shown in the respective line in column 5 of Table I, application no. 17, has been published in F. And the activity of the gene product thereof is the activity of CBL-interacting protein kinase.

Accordingly, in one embodiment, the process of the present invention for producing fructose in a non-human organism, like a microorganism or a plant or a part thereof, comprises increasing or generating the activity of a gene product with the activity of a gene product conferring the activity of “CBL-interacting protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said At2g34180, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At2g34180, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At2g34180, or a functional equivalent or a homolog thereof as depicted in column 8 of Table II, application no. 17, preferably a homolog or functional equivalent as depicted in column 8 of Table II B, application no. 17, and being depicted in the same respective line as said At2g34180, and preferably the activity is increased non-targeted, whereby the respective line disclose in column 7 the fine chemical fructose.

Accordingly, in one embodiment, the molecule which activity is to be increased in the process of the invention is the gene product with an activity as a “CBL-interacting protein kinase”, preferably it is the molecule of section (a) or (b) of this paragraph.

In particular, it was observed that in plants, especially in Arabidopsis thaliana, increasing or generating the activity of a gene product non-targeted with the activity of a “CBL-interacting protein kinase”, preferably being encoded by a gene comprising the nucleic acid sequence SEQ ID NO. 20578, preferably the coding region thereof, conferred the production of or the increase in fructose compared with the wild type control.

The nucleic acid sequence of At2g34180 from Arabidopsis thaliana, e.g. as shown in the respective line in column 5 of Table I, application no. 17, has been published in F. And the activity of the gene product thereof is the activity of CBL-interacting protein kinase.

Accordingly, in one embodiment, the process of the present invention for producing glucose in a non-human organism, like a microorganism or a plant or a part thereof, comprises increasing or generating the activity of a gene product with the activity of a gene product conferring the activity of “CBL-interacting protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said At2g34180, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At2g34180, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At2g34180, or a functional equivalent or a homolog thereof as depicted in column 8 of Table II, application no. 17, preferably a homolog or functional equivalent as depicted in column 8 of Table II B, application no. 17, and being depicted in the same respective line as said At2g34180, and preferably the activity is increased non-targeted, whereby the respective line disclose in column 7 the fine chemical glucose.

Accordingly, in one embodiment, the molecule which activity is to be increased in the process of the invention is the gene product with an activity as a “CBL-interacting protein kinase”, preferably it is the molecule of section (a) or (b) of this paragraph.

In particular, it was observed that in plants, especially in Arabidopsis thaliana, increasing or generating the activity of a gene product non-targeted with the activity of a “CBL-interacting protein kinase”, preferably being encoded by a gene comprising the nucleic acid sequence SEQ ID NO. 20578, preferably the coding region thereof, conferred the production of or the increase in glucose compared with the wild type control.

The nucleic acid sequence of At2g34180 from Arabidopsis thaliana, e.g. as shown in the respective line in column 5 of Table I, application no. 17, has been published in F. And the activity of the gene product thereof is the activity of CBL-interacting protein kinase.

Accordingly, in one embodiment, the process of the present invention for producing sedoheptulose-7-phosphate in a non-human organism, like a microorganism or a plant or a part thereof, comprises increasing or generating the activity of a gene product with the activity of a gene product conferring the activity of “CBL-interacting protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical sedoheptulose-7-phosphate), application no. 17, preferably the coding region thereof, or a homolog or a fragment thereof, and being depicted in the same respective line as said At2g34180, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At2g34180, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At2g34180, or a functional equivalent or a homolog thereof as depicted in column 8 of Table II, application no. 17, preferably a homolog or functional equivalent as depicted in column 8 of Table II B, application no. 17, and being depicted in the same respective line as said At2g34180, and preferably the activity is increased non-targeted, whereby the respective line disclose in column 7 the fine chemical sedoheptulose-7-phosphate.

Accordingly, in one embodiment, the molecule which activity is to be increased in the process of the invention is the gene product with an activity as a “CBL-interacting protein kinase”, preferably it is the molecule of section (a) or (b) of this paragraph.

In particular, it was observed that in plants, especially in Arabidopsis thaliana, increasing or generating the activity of a gene product non-targeted with the activity of a “CBL-interacting protein kinase”, preferably being encoded by a gene comprising the nucleic acid sequence SEQ ID NO. 20578, preferably the coding region thereof, conferred the production of or the increase in sedoheptulose-7-phosphate compared with the wild type control.

The nucleic acid sequence of At2g37040 from Arabidopsis thaliana, e.g. as shown in the respective line in column 5 of Table I, application no. 17, has been published in F. And the activity of the gene product thereof is the activity of phenylalanine ammonia-lyase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “phenylalanine ammonia-lyase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said At2g37040, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At2g37040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At2g37040, or a functional equivalent or a homolog thereof as depicted in column 8 of Table II, application no. 17, preferably a homolog or functional equivalent as depicted in column 8 of Table II B, application no. 17, and being depicted in the same respective line as said At2g37040, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “phenylalanine ammonia-lyase”, preferably it is the molecule of section (a) or (b) of this paragraph.

In particular, it was observed that in plants, especially in Arabidopsis thaliana, increasing or generating the activity of a gene product non-targeted with the activity of a “phenylalanine ammonia-lyase”, preferably being encoded by a gene comprising the nucleic acid sequence SEQ ID NO. 133042, preferably the coding region thereof, conferred the production of or the increase in glucose-1-phosphate compared with the wild type control.

The nucleic acid sequence of At2g42830 from Arabidopsis thaliana, e.g. as shown in the respective line in column 5 of Table I, application no. 17, has been published in F. And the activity of the gene product thereof is the activity of MADS box protein transcription factor.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “MADS box protein transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said At2g42830, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At2g42830, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At2g42830, or a functional equivalent or a homolog thereof as depicted in column 8 of Table II, application no. 17, preferably a homolog or functional equivalent as depicted in column 8 of Table II B, application no. 17, and being depicted in the same respective line as said At2g42830, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “MADS box protein transcription factor”, preferably it is the molecule of section (a) or (b) of this paragraph.

In particular, it was observed that in plants, especially in Arabidopsis thaliana, increasing or generating the activity of a gene product non-targeted with the activity of a “MADS box protein transcription factor”, preferably being encoded by a gene comprising the nucleic acid sequence SEQ ID NO. 108727, preferably the coding region thereof, conferred the production of or the increase in glucose-1-phosphate compared with the wild type control.

The nucleic acid sequence of At2g45420 from Arabidopsis thaliana, e.g. as shown in the respective line in column 5 of Table I, application no. 17, has been published in F. And the activity of the gene product thereof is the activity of At2g45420-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “At2g45420-protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said At2g45420, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At2g45420, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At2g45420, or a functional equivalent or a homolog thereof as depicted in column 8 of Table II, application no. 17, preferably a homolog or functional equivalent as depicted in column 8 of Table II B, application no. 17, and being depicted in the same respective line as said At2g45420, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “At2g45420-protein”, preferably it is the molecule of section (a) or (b) of this paragraph.

In particular, it was observed that in plants, especially in Arabidopsis thaliana, increasing or generating the activity of a gene product non-targeted with the activity of a “At2g45420-protein”, preferably being encoded by a gene comprising the nucleic acid sequence SEQ ID NO. 1623, preferably the coding region thereof, conferred the production of or the increase in myo-inositol compared with the wild type control.

The nucleic acid sequence of At2g46500 from Arabidopsis thaliana, e.g. as shown in the respective line in column 5 of Table I, application no. 17, has been published in F. And the activity of the gene product thereof is the activity of phosphatidylinositol 3- and 4-kinase family protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “phosphatidylinositol 3- and 4-kinase family protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said At2g46500, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At2g46500, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At2g46500, or a functional equivalent or a homolog thereof as depicted in column 8 of Table II, application no. 17, preferably a homolog or functional equivalent as depicted in column 8 of Table II B, application no. 17, and being depicted in the same respective line as said At2g46500, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “phosphatidylinositol 3- and 4-kinase family protein”, preferably it is the molecule of section (a) or (b) of this paragraph.

In particular, it was observed that in plants, especially in Arabidopsis thaliana, increasing or generating the activity of a gene product non-targeted with the activity of a “phosphatidylinositol 3- and 4-kinase family protein”, preferably being encoded by a gene comprising the nucleic acid sequence SEQ ID NO. 21106, preferably the coding region thereof, conferred the production of or the increase in glucose-1-phosphate compared with the wild type control.

The nucleic acid sequence of At2g47180 from Arabidopsis thaliana, e.g. as shown in the respective line in column 5 of Table I, application no. 17, has been published in F. And the activity of the gene product thereof is the activity of galactinol synthase.

Accordingly, in one embodiment, the process of the present invention for producing raffinose in a non-human organism, like a microorganism or a plant or a part thereof, comprises increasing or generating the activity of a gene product with the activity of a gene product conferring the activity of “galactinol synthase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said At2g47180, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At2g47180, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At2g47180, or a functional equivalent or a homolog thereof as depicted in column 8 of Table II, application no. 17, preferably a homolog or functional equivalent as depicted in column 8 of Table II B, application no. 17, and being depicted in the same respective line as said At2g47180, and preferably the activity is increased non-targeted, whereby the respective line disclose in column 7 the fine chemical raffinose.

Accordingly, in one embodiment, the molecule which activity is to be increased in the process of the invention is the gene product with an activity as a “galactinol synthase”, preferably it is the molecule of section (a) or (b) of this paragraph.

In particular, it was observed that in plants, especially in Arabidopsis thaliana, increasing or generating the activity of a gene product non-targeted with the activity of a “galactinol synthase”, preferably being encoded by a gene comprising the nucleic acid sequence SEQ ID NO. 141637, preferably the coding region thereof, conferred the production of or the increase in raffinose compared with the wild type control.

The nucleic acid sequence of At2g47180 from Arabidopsis thaliana, e.g. as shown in the respective line in column 5 of Table I, application no. 17, has been published in F. And the activity of the gene product thereof is the activity of galactinol synthase.

Accordingly, in one embodiment, the process of the present invention for producing raffinose in a non-human organism, like a microorganism or a plant or a part thereof, comprises increasing or generating the activity of a gene product with the activity of a gene product conferring the activity of “galactinol synthase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said At2g47180, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At2g47180, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At2g47180, or a functional equivalent or a homolog thereof as depicted in column 8 of Table II, application no. 17, preferably a homolog or functional equivalent as depicted in column 8 of Table II B, application no. 17, and being depicted in the same respective line as said At2g47180, and preferably the activity is increased non-targeted, whereby the respective line disclose in column 7 the fine chemical raffinose.

Accordingly, in one embodiment, the molecule which activity is to be increased in the process of the invention is the gene product with an activity as a “galactinol synthase”, preferably it is the molecule of section (a) or (b) of this paragraph.

In particular, it was observed that in plants, especially in Arabidopsis thaliana, increasing or generating the activity of a gene product non-targeted with the activity of a “galactinol synthase”, preferably being encoded by a gene comprising the nucleic acid sequence SEQ ID NO. 141637, preferably the coding region thereof, conferred the production of or the increase in raffinose compared with the wild type control.

The nucleic acid sequence of At3g02990 from Arabidopsis thaliana, e.g. as shown in the respective line in column 5 of Table I, application no. 17, has been published in F. And the activity of the gene product thereof is the activity of heat shock transcription factor.

Accordingly, in one embodiment, the process of the present invention for producing fructose in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said At3g02990, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g02990, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g02990, or a functional equivalent or a homolog thereof as depicted in column 8 of Table II, application no. 17, preferably a homolog or functional equivalent as depicted in column 8 of Table II B, application no. 17, and being depicted in the same respective line as said At3g02990, and preferably the activity is increased non-targeted, whereby the respective line disclose in column 7 the fine chemical fructose.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 68657, preferably the coding region thereof, conferred the production of or the increase in fructose compared with the wild type control.

The nucleic acid sequence of At3g02990 from Arabidopsis thaliana, e.g. as shown in the respective line in column 5 of Table I, application no. 17, has been published in F. And the activity of the gene product thereof is the activity of heat shock transcription factor.

Accordingly, in one embodiment, the process of the present invention for producing glucose in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said At3g02990, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g02990, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g02990, or a functional equivalent or a homolog thereof as depicted in column 8 of Table II, application no. 17, preferably a homolog or functional equivalent as depicted in column 8 of Table II B, application no. 17, and being depicted in the same respective line as said At3g02990, and preferably the activity is increased non-targeted, whereby the respective line disclose in column 7 the fine chemical glucose.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 68657, preferably the coding region thereof, conferred the production of or the increase in glucose compared with the wild type control.

The nucleic acid sequence of At3g02990 from Arabidopsis thaliana, e.g. as shown in the respective line in column 5 of Table I, application no. 17, has been published in F. And the activity of the gene product thereof is the activity of heat shock transcription factor.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said At3g02990, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g02990, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g02990, or a functional equivalent or a homolog thereof as depicted in column 8 of Table II, application no. 17, preferably a homolog or functional equivalent as depicted in column 8 of Table II B, application no. 17, and being depicted in the same respective line as said At3g02990, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 68657, preferably the coding region thereof, conferred the production of or the increase in myo-inositol compared with the wild type control.

The nucleic acid sequence of At3g02990 from Arabidopsis thaliana, e.g. as shown in the respective line in column 5 of Table I, application no. 17, has been published in F. And the activity of the gene product thereof is the activity of heat shock transcription factor.

Accordingly, in one embodiment, the process of the present invention for producing raffinose in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said At3g02990, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g02990, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g02990, or a functional equivalent or a homolog thereof as depicted in column 8 of Table II, application no. 17, preferably a homolog or functional equivalent as depicted in column 8 of Table II B, application no. 17, and being depicted in the same respective line as said At3g02990, and preferably the activity is increased non-targeted, whereby the respective line disclose in column 7 the fine chemical raffinose.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 68657, preferably the coding region thereof, conferred the production of or the increase in raffinose compared with the wild type control.

The nucleic acid sequence of At3g04050 from Arabidopsis thaliana, e.g. as shown in the respective line in column 5 of Table I, application no. 17, has been published in F. And the activity of the gene product thereof is the activity of pyruvate kinase.

Accordingly, in one embodiment, the process of the present invention for producing fructose in a non-human organism, like a microorganism or a plant or a part thereof, comprises increasing or generating the activity of a gene product with the activity of a gene product conferring the activity of “pyruvate kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said At3g04050, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g04050, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g04050, or a functional equivalent or a homolog thereof as depicted in column 8 of Table II, application no. 17, preferably a homolog or functional equivalent as depicted in column 8 of Table II B, application no. 17, and being depicted in the same respective line as said At3g04050, and preferably the activity is increased plastidic, whereby the respective line disclose in column 7 the fine chemical fructose.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 21497, preferably the coding region thereof, conferred the production of or the increase in fructose compared with the wild type control.

The nucleic acid sequence of At3g06270 from Arabidopsis thaliana, e.g. as shown in the respective line in column 5 of Table I, application no. 17, has been published in F. And the activity of the gene product thereof is the activity of protein phosphatase.

Accordingly, in one embodiment, the process of the present invention for producing glucose in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said At3g06270, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g06270, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g06270, or a functional equivalent or a homolog thereof as depicted in column 8 of Table II, application no. 17, preferably a homolog or functional equivalent as depicted in column 8 of Table II B, application no. 17, and being depicted in the same respective line as said At3g06270, and preferably the activity is increased non-targeted, whereby the respective line disclose in column 7 the fine chemical glucose.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 22015, preferably the coding region thereof, conferred the production of or the increase in glucose compared with the wild type control.

The nucleic acid sequence of At3g06270 from Arabidopsis thaliana, e.g. as shown in the respective line in column 5 of Table I, application no. 17, has been published in F. And the activity of the gene product thereof is the activity of protein phosphatase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said At3g06270, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g06270, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g06270, or a functional equivalent or a homolog thereof as depicted in column 8 of Table II, application no. 17, preferably a homolog or functional equivalent as depicted in column 8 of Table II B, application no. 17, and being depicted in the same respective line as said At3g06270, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 22015, preferably the coding region thereof, conferred the production of or the increase in myo-inositol compared with the wild type control.

The nucleic acid sequence of At3g08710 from Arabidopsis thaliana, e.g. as shown in the respective line in column 5 of Table I, application no. 17, has been published in F. And the activity of the gene product thereof is the activity of thioredoxin family protein.

Accordingly, in one embodiment, the process of the present invention for producing fructose in a non-human organism, like a microorganism or a plant or a part thereof, comprises increasing or generating the activity of a gene product with the activity of a gene product conferring the activity of “thioredoxin family protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said At3g08710, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g08710, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g08710, or a functional equivalent or a homolog thereof as depicted in column 8 of Table II, application no. 17, preferably a homolog or functional equivalent as depicted in column 8 of Table II B, application no. 17, and being depicted in the same respective line as said At3g08710, and preferably the activity is increased non-targeted, whereby the respective line disclose in column 7 the fine chemical fructose.

Accordingly, in one embodiment, the molecule which activity is to be increased in the process of the invention is the gene product with an activity as a “thioredoxin family protein”, preferably it is the molecule of section (a) or (b) of this paragraph.

In particular, it was observed that in plants, especially in Arabidopsis thaliana, increasing or generating the activity of a gene product non-targeted with the activity of a “thioredoxin family protein”, preferably being encoded by a gene comprising the nucleic acid sequence SEQ ID NO. 22249, preferably the coding region thereof, conferred the production of or the increase in fructose compared with the wild type control.

The nucleic acid sequence of At3g08710 from Arabidopsis thaliana, e.g. as shown in the respective line in column 5 of Table I, application no. 17, has been published in F. And the activity of the gene product thereof is the activity of thioredoxin family protein.

Accordingly, in one embodiment, the process of the present invention for producing glucose in a non-human organism, like a microorganism or a plant or a part thereof, comprises increasing or generating the activity of a gene product with the activity of a gene product conferring the activity of “thioredoxin family protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said At3g08710, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g08710, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g08710, or a functional equivalent or a homolog thereof as depicted in column 8 of Table II, application no. 17, preferably a homolog or functional equivalent as depicted in column 8 of Table II B, application no. 17, and being depicted in the same respective line as said At3g08710, and preferably the activity is increased non-targeted, whereby the respective line disclose in column 7 the fine chemical glucose.

Accordingly, in one embodiment, the molecule which activity is to be increased in the process of the invention is the gene product with an activity as a “thioredoxin family protein”, preferably it is the molecule of section (a) or (b) of this paragraph.

In particular, it was observed that in plants, especially in Arabidopsis thaliana, increasing or generating the activity of a gene product non-targeted with the activity of a “thioredoxin family protein”, preferably being encoded by a gene comprising the nucleic acid sequence SEQ ID NO. 22249, preferably the coding region thereof, conferred the production of or the increase in glucose compared with the wild type control.

The nucleic acid sequence of At3g08710 from Arabidopsis thaliana, e.g. as shown in the respective line in column 5 of Table I, application no. 17, has been published in F. And the activity of the gene product thereof is the activity of thioredoxin family protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “thioredoxin family protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said At3g08710, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g08710, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g08710, or a functional equivalent or a homolog thereof as depicted in column 8 of Table II, application no. 17, preferably a homolog or functional equivalent as depicted in column 8 of Table II B, application no. 17, and being depicted in the same respective line as said At3g08710, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “thioredoxin family protein”, preferably it is the molecule of section (a) or (b) of this paragraph.

In particular, it was observed that in plants, especially in Arabidopsis thaliana, increasing or generating the activity of a gene product non-targeted with the activity of a “thioredoxin family protein”, preferably being encoded by a gene comprising the nucleic acid sequence SEQ ID NO. 22249, preferably the coding region thereof, conferred the production of or the increase in myo-inositol compared with the wild type control.

The nucleic acid sequence of At3g14230 from Arabidopsis thaliana, e.g. as shown in the respective line in column 5 of Table I, application no. 17, has been published in F. And the activity of the gene product thereof is the activity of DNA binding protein.

Accordingly, in one embodiment, the process of the present invention for producing saccharic acid in a non-human organism, like a microorganism or a plant or a part thereof, comprises increasing or generating the activity of a gene product with the activity of a gene product conferring the activity of “DNA binding protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical saccharic acid), application no. 17, preferably the coding region thereof, or a homolog or a fragment thereof, and being depicted in the same respective line as said At3g14230, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g14230, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g14230, or a functional equivalent or a homolog thereof as depicted in column 8 of Table II, application no. 17, preferably a homolog or functional equivalent as depicted in column 8 of Table II B, application no. 17, and being depicted in the same respective line as said At3g14230, and preferably the activity is increased plastidic, whereby the respective line disclose in column 7 the fine chemical saccharic acid.

Accordingly, in one embodiment, the molecule which activity is to be increased in the process of the invention is the gene product with an activity as a “DNA binding protein”, preferably it is the molecule of section (a) or (b) of this paragraph.

In particular, it was observed that in plants, especially in Arabidopsis thaliana, increasing or generating the activity of a gene product plastidic with the activity of a “DNA binding protein”, preferably being encoded by a gene comprising the nucleic acid sequence SEQ ID NO. 22699, preferably the coding region thereof, conferred the production of or the increase in saccharic acid compared with the wild type control.

The nucleic acid sequence of At3g20910 from Arabidopsis thaliana, e.g. as shown in the respective line in column 5 of Table I, application no. 17, has been published in F. And the activity of the gene product thereof is the activity of CCAAT-binding transcription factor.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “CCAAT-binding transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said At3g20910, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g20910, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g20910, or a functional equivalent or a homolog thereof as depicted in column 8 of Table II, application no. 17, preferably a homolog or functional equivalent as depicted in column 8 of Table II B, application no. 17, and being depicted in the same respective line as said At3g20910, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “CCAAT-binding transcription factor”, preferably it is the molecule of section (a) or (b) of this paragraph.

In particular, it was observed that in plants, especially in Arabidopsis thaliana, increasing or generating the activity of a gene product non-targeted with the activity of a “CCAAT-binding transcription factor”, preferably being encoded by a gene comprising the nucleic acid sequence SEQ ID NO. 22921, preferably the coding region thereof, conferred the production of or the increase in glucose-1-phosphate compared with the wild type control.

The nucleic acid sequence of At3g20910 from Arabidopsis thaliana, e.g. as shown in the respective line in column 5 of Table I, application no. 17, has been published in F. And the activity of the gene product thereof is the activity of CCAAT-binding transcription factor.

Accordingly, in one embodiment, the process of the present invention for producing raffinose in a non-human organism, like a microorganism or a plant or a part thereof, comprises increasing or generating the activity of a gene product with the activity of a gene product conferring the activity of “CCAAT-binding transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said At3g20910, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g20910, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g20910, or a functional equivalent or a homolog thereof as depicted in column 8 of Table II, application no. 17, preferably a homolog or functional equivalent as depicted in column 8 of Table II B, application no. 17, and being depicted in the same respective line as said At3g20910, and preferably the activity is increased non-targeted, whereby the respective line disclose in column 7 the fine chemical raffinose.

Accordingly, in one embodiment, the molecule which activity is to be increased in the process of the invention is the gene product with an activity as a “CCAAT-binding transcription factor”, preferably it is the molecule of section (a) or (b) of this paragraph.

In particular, it was observed that in plants, especially in Arabidopsis thaliana, increasing or generating the activity of a gene product non-targeted with the activity of a “CCAAT-binding transcription factor”, preferably being encoded by a gene comprising the nucleic acid sequence SEQ ID NO. 22921, preferably the coding region thereof, conferred the production of or the increase in raffinose compared with the wild type control.

The nucleic acid sequence of At3g23000 from Arabidopsis thaliana, e.g. as shown in the respective line in column 5 of Table I, application no. 17, has been published in F. And the activity of the gene product thereof is the activity of protein kinase.

Accordingly, in one embodiment, the process of the present invention for producing fructose in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said At3g23000, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g23000, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g23000, or a functional equivalent or a homolog thereof as depicted in column 8 of Table II, application no. 17, preferably a homolog or functional equivalent as depicted in column 8 of Table II B, application no. 17, and being depicted in the same respective line as said At3g23000, and preferably the activity is increased non-targeted, whereby the respective line disclose in column 7 the fine chemical fructose.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 1815, preferably the coding region thereof, conferred the production of or the increase in fructose compared with the wild type control.

The nucleic acid sequence of At3g23000 from Arabidopsis thaliana, e.g. as shown in the respective line in column 5 of Table I, application no. 17, has been published in F. And the activity of the gene product thereof is the activity of protein kinase.

Accordingly, in one embodiment, the process of the present invention for producing glucose in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said At3g23000, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g23000, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g23000, or a functional equivalent or a homolog thereof as depicted in column 8 of Table II, application no. 17, preferably a homolog or functional equivalent as depicted in column 8 of Table II B, application no. 17, and being depicted in the same respective line as said At3g23000, and preferably the activity is increased non-targeted, whereby the respective line disclose in column 7 the fine chemical glucose.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 1815, preferably the coding region thereof, conferred the production of or the increase in glucose compared with the wild type control.

The nucleic acid sequence of At3g23000 from Arabidopsis thaliana, e.g. as shown in the respective line in column 5 of Table I, application no. 17, has been published in F. And the activity of the gene product thereof is the activity of protein kinase.

Accordingly, in one embodiment, the process of the present invention for producing sedoheptulose-7-phosphate in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical sedoheptulose-7-phosphate), application no. 17, preferably the coding region thereof, or a homolog or a fragment thereof, and being depicted in the same respective line as said At3g23000, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g23000, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g23000, or a functional equivalent or a homolog thereof as depicted in column 8 of Table II, application no. 17, preferably a homolog or functional equivalent as depicted in column 8 of Table II B, application no. 17, and being depicted in the same respective line as said At3g23000, and preferably the activity is increased non-targeted, whereby the respective line disclose in column 7 the fine chemical sedoheptulose-7-phosphate.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 1815, preferably the coding region thereof, conferred the production of or the increase in sedoheptulose-7-phosphate compared with the wild type control.

The nucleic acid sequence of At3g26400 from Arabidopsis thaliana, e.g. as shown in the respective line in column 5 of Table I, application no. 17, has been published in F. And the activity of the gene product thereof is the activity of eukaryotic translation initiation factor.

Accordingly, in one embodiment, the process of the present invention for producing fructose in a non-human organism, like a microorganism or a plant or a part thereof, comprises increasing or generating the activity of a gene product with the activity of a gene product conferring the activity of “eukaryotic translation initiation factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said At3g26400, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g26400, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g26400, or a functional equivalent or a homolog thereof as depicted in column 8 of Table II, application no. 17, preferably a homolog or functional equivalent as depicted in column 8 of Table II B, application no. 17, and being depicted in the same respective line as said At3g26400, and preferably the activity is increased non-targeted, whereby the respective line disclose in column 7 the fine chemical fructose.

Accordingly, in one embodiment, the molecule which activity is to be increased in the process of the invention is the gene product with an activity as a “eukaryotic translation initiation factor”, preferably it is the molecule of section (a) or (b) of this paragraph.

In particular, it was observed that in plants, especially in Arabidopsis thaliana, increasing or generating the activity of a gene product non-targeted with the activity of a “eukaryotic translation initiation factor”, preferably being encoded by a gene comprising the nucleic acid sequence SEQ ID NO. 68727, preferably the coding region thereof, conferred the production of or the increase in fructose compared with the wild type control.

The nucleic acid sequence of At3g26400 from Arabidopsis thaliana, e.g. as shown in the respective line in column 5 of Table I, application no. 17, has been published in F. And the activity of the gene product thereof is the activity of eukaryotic translation initiation factor.

Accordingly, in one embodiment, the process of the present invention for producing glucose in a non-human organism, like a microorganism or a plant or a part thereof, comprises increasing or generating the activity of a gene product with the activity of a gene product conferring the activity of “eukaryotic translation initiation factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said At3g26400, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g26400, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g26400, or a functional equivalent or a homolog thereof as depicted in column 8 of Table II, application no. 17, preferably a homolog or functional equivalent as depicted in column 8 of Table II B, application no. 17, and being depicted in the same respective line as said At3g26400, and preferably the activity is increased non-targeted, whereby the respective line disclose in column 7 the fine chemical glucose.

Accordingly, in one embodiment, the molecule which activity is to be increased in the process of the invention is the gene product with an activity as a “eukaryotic translation initiation factor”, preferably it is the molecule of section (a) or (b) of this paragraph.

In particular, it was observed that in plants, especially in Arabidopsis thaliana, increasing or generating the activity of a gene product non-targeted with the activity of a “eukaryotic translation initiation factor”, preferably being encoded by a gene comprising the nucleic acid sequence SEQ ID NO. 68727, preferably the coding region thereof, conferred the production of or the increase in glucose compared with the wild type control.

The nucleic acid sequence of At3g26400 from Arabidopsis thaliana, e.g. as shown in the respective line in column 5 of Table I, application no. 17, has been published in F. And the activity of the gene product thereof is the activity of eukaryotic translation initiation factor.

Accordingly, in one embodiment, the process of the present invention for producing raffinose in a non-human organism, like a microorganism or a plant or a part thereof, comprises increasing or generating the activity of a gene product with the activity of a gene product conferring the activity of “eukaryotic translation initiation factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said At3g26400, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g26400, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g26400, or a functional equivalent or a homolog thereof as depicted in column 8 of Table II, application no. 17, preferably a homolog or functional equivalent as depicted in column 8 of Table II B, application no. 17, and being depicted in the same respective line as said At3g26400, and preferably the activity is increased non-targeted, whereby the respective line disclose in column 7 the fine chemical raffinose.

Accordingly, in one embodiment, the molecule which activity is to be increased in the process of the invention is the gene product with an activity as a “eukaryotic translation initiation factor”, preferably it is the molecule of section (a) or (b) of this paragraph.

In particular, it was observed that in plants, especially in Arabidopsis thaliana, increasing or generating the activity of a gene product non-targeted with the activity of a “eukaryotic translation initiation factor”, preferably being encoded by a gene comprising the nucleic acid sequence SEQ ID NO. 68727, preferably the coding region thereof, conferred the production of or the increase in raffinose compared with the wild type control.

The nucleic acid sequence of At3g59790 from Arabidopsis thaliana, e.g. as shown in the respective line in column 5 of Table I, application no. 17, has been published in F. And the activity of the gene product thereof is the activity of mitogen-activated protein kinase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “mitogen-activated protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said At3g59790, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g59790, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g59790, or a functional equivalent or a homolog thereof as depicted in column 8 of Table II, application no. 17, preferably a homolog or functional equivalent as depicted in column 8 of Table II B, application no. 17, and being depicted in the same respective line as said At3g59790, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “mitogen-activated protein kinase”, preferably it is the molecule of section (a) or (b) of this paragraph.

In particular, it was observed that in plants, especially in Arabidopsis thaliana, increasing or generating the activity of a gene product non-targeted with the activity of a “mitogen-activated protein kinase”, preferably being encoded by a gene comprising the nucleic acid sequence SEQ ID NO. 141758, preferably the coding region thereof, conferred the production of or the increase in myo-inositol compared with the wild type control.

The nucleic acid sequence of At3g61830 from Arabidopsis thaliana, e.g. as shown in the respective line in column 5 of Table I, application no. 17, has been published in F. And the activity of the gene product thereof is the activity of auxin response factor.

Accordingly, in one embodiment, the process of the present invention for producing sedoheptulose-7-phosphate in a non-human organism, like a microorganism or a plant or a part thereof, comprises increasing or generating the activity of a gene product with the activity of a gene product conferring the activity of “auxin response factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical sedoheptulose-7-phosphate), application no. 17, preferably the coding region thereof, or a homolog or a fragment thereof, and being depicted in the same respective line as said At3g61830, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g61830, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g61830, or a functional equivalent or a homolog thereof as depicted in column 8 of Table II, application no. 17, preferably a homolog or functional equivalent as depicted in column 8 of Table II B, application no. 17, and being depicted in the same respective line as said At3g61830, and preferably the activity is increased non-targeted, whereby the respective line disclose in column 7 the fine chemical sedoheptulose-7-phosphate.

Accordingly, in one embodiment, the molecule which activity is to be increased in the process of the invention is the gene product with an activity as a “auxin response factor”, preferably it is the molecule of section (a) or (b) of this paragraph.

In particular, it was observed that in plants, especially in Arabidopsis thaliana, increasing or generating the activity of a gene product non-targeted with the activity of a “auxin response factor”, preferably being encoded by a gene comprising the nucleic acid sequence SEQ ID NO. 2367, preferably the coding region thereof, conferred the production of or the increase in sedoheptulose-7-phosphate compared with the wild type control.

The nucleic acid sequence of At3g62930 from Arabidopsis thaliana, e.g. as shown in the respective line in column 5 of Table I, application no. 17, has been published in F. And the activity of the gene product thereof is the activity of monthiol glutaredoxin.

Accordingly, in one embodiment, the process of the present invention for producing fructose in a non-human organism, like a microorganism or a plant or a part thereof, comprises increasing or generating the activity of a gene product with the activity of a gene product conferring the activity of “monthiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said At3g62930, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g62930, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g62930, or a functional equivalent or a homolog thereof as depicted in column 8 of Table II, application no. 17, preferably a homolog or functional equivalent as depicted in column 8 of Table II B, application no. 17, and being depicted in the same respective line as said At3g62930, and preferably the activity is increased non-targeted, whereby the respective line disclose in column 7 the fine chemical fructose.

Accordingly, in one embodiment, the molecule which activity is to be increased in the process of the invention is the gene product with an activity as a “monthiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monthiol glutaredoxin”, preferably being encoded by a gene comprising the nucleic acid sequence SEQ ID NO. 2573, preferably the coding region thereof, conferred the production of or the increase in fructose compared with the wild type control.

The nucleic acid sequence of At3g62930 from Arabidopsis thaliana, e.g. as shown in the respective line in column 5 of Table I, application no. 17, has been published in F. And the activity of the gene product thereof is the activity of monthiol glutaredoxin.

Accordingly, in one embodiment, the process of the present invention for producing glucose in a non-human organism, like a microorganism or a plant or a part thereof, comprises increasing or generating the activity of a gene product with the activity of a gene product conferring the activity of “monthiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said At3g62930, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g62930, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g62930, or a functional equivalent or a homolog thereof as depicted in column 8 of Table II, application no. 17, preferably a homolog or functional equivalent as depicted in column 8 of Table II B, application no. 17, and being depicted in the same respective line as said At3g62930, and preferably the activity is increased non-targeted, whereby the respective line disclose in column 7 the fine chemical glucose.

Accordingly, in one embodiment, the molecule which activity is to be increased in the process of the invention is the gene product with an activity as a “monthiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monthiol glutaredoxin”, preferably being encoded by a gene comprising the nucleic acid sequence SEQ ID NO. 2573, preferably the coding region thereof, conferred the production of or the increase in glucose compared with the wild type control.

The nucleic acid sequence of At3g62930 from Arabidopsis thaliana, e.g. as shown in the respective line in column 5 of Table I, application no. 17, has been published in F. And the activity of the gene product thereof is the activity of monthiol glutaredoxin.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “monthiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said At3g62930, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g62930, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g62930, or a functional equivalent or a homolog thereof as depicted in column 8 of Table II, application no. 17, preferably a homolog or functional equivalent as depicted in column 8 of Table II B, application no. 17, and being depicted in the same respective line as said At3g62930, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “monthiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monthiol glutaredoxin”, preferably being encoded by a gene comprising the nucleic acid sequence SEQ ID NO. 2573, preferably the coding region thereof, conferred the production of or the increase in glucose-1-phosphate compared with the wild type control.

The nucleic acid sequence of At3g62930 from Arabidopsis thaliana, e.g. as shown in the respective line in column 5 of Table I, application no. 17, has been published in F. And the activity of the gene product thereof is the activity of monthiol glutaredoxin.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “monthiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said At3g62930, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g62930, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g62930, or a functional equivalent or a homolog thereof as depicted in column 8 of Table II, application no. 17, preferably a homolog or functional equivalent as depicted in column 8 of Table II B, application no. 17, and being depicted in the same respective line as said At3g62930, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “monthiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monthiol glutaredoxin”, preferably being encoded by a gene comprising the nucleic acid sequence SEQ ID NO. 2573, preferably the coding region thereof, conferred the production of or the increase in myo-inositol compared with the wild type control.

The nucleic acid sequence of At3g62930 from Arabidopsis thaliana, e.g. as shown in the respective line in column 5 of Table I, application no. 17, has been published in F. And the activity of the gene product thereof is the activity of monthiol glutaredoxin.

Accordingly, in one embodiment, the process of the present invention for producing sedoheptulose-7-phosphate in a non-human organism, like a microorganism or a plant or a part thereof, comprises increasing or generating the activity of a gene product with the activity of a gene product conferring the activity of “monthiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical sedoheptulose-7-phosphate), application no. 17, preferably the coding region thereof, or a homolog or a fragment thereof, and being depicted in the same respective line as said At3g62930, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g62930, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g62930, or a functional equivalent or a homolog thereof as depicted in column 8 of Table II, application no. 17, preferably a homolog or functional equivalent as depicted in column 8 of Table II B, application no. 17, and being depicted in the same respective line as said At3g62930, and preferably the activity is increased non-targeted, whereby the respective line disclose in column 7 the fine chemical sedoheptulose-7-phosphate.

Accordingly, in one embodiment, the molecule which activity is to be increased in the process of the invention is the gene product with an activity as a “monthiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monthiol glutaredoxin”, preferably being encoded by a gene comprising the nucleic acid sequence SEQ ID NO. 2573, preferably the coding region thereof, conferred the production of or the increase in sedoheptulose-7-phosphate compared with the wild type control.

The nucleic acid sequence of At3g62930 from Arabidopsis thaliana, e.g. as shown in the respective line in column 5 of Table I, application no. 17, has been published in F. And the activity of the gene product thereof is the activity of monthiol glutaredoxin.

Accordingly, in one embodiment, the process of the present invention for producing sucrose in a non-human organism, like a microorganism or a plant or a part thereof, comprises increasing or generating the activity of a gene product with the activity of a gene product conferring the activity of “monthiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said At3g62930, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g62930, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g62930, or a functional equivalent or a homolog thereof as depicted in column 8 of Table II, application no. 17, preferably a homolog or functional equivalent as depicted in column 8 of Table II B, application no. 17, and being depicted in the same respective line as said At3g62930, and preferably the activity is increased non-targeted, whereby the respective line disclose in column 7 the fine chemical sucrose.

Accordingly, in one embodiment, the molecule which activity is to be increased in the process of the invention is the gene product with an activity as a “monthiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monthiol glutaredoxin”, preferably being encoded by a gene comprising the nucleic acid sequence SEQ ID NO. 2573, preferably the coding region thereof, conferred the production of or the increase in sucrose compared with the wild type control.

The nucleic acid sequence of At3g62950 from Arabidopsis thaliana, e.g. as shown in the respective line in column 5 of Table I, application no. 17, has been published in F. And the activity of the gene product thereof is the activity of glutaredoxin.

Accordingly, in one embodiment, the process of the present invention for producing fructose in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said At3g62950, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g62950, or a functional equivalent or a homolog thereof as depicted in column 8 of Table II, application no. 17, preferably a homolog or functional equivalent as depicted in column 8 of Table II B, application no. 17, and being depicted in the same respective line as said At3g62950, and preferably the activity is increased non-targeted, whereby the respective line disclose in column 7 the fine chemical fructose.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in fructose compared with the wild type control.

The nucleic acid sequence of At3g62950 from Arabidopsis thaliana, e.g. as shown in the respective line in column 5 of Table I, application no. 17, has been published in F. And the activity of the gene product thereof is the activity of glutaredoxin.

Accordingly, in one embodiment, the process of the present invention for producing glucose in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said At3g62950, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g62950, or a functional equivalent or a homolog thereof as depicted in column 8 of Table II, application no. 17, preferably a homolog or functional equivalent as depicted in column 8 of Table II B, application no. 17, and being depicted in the same respective line as said At3g62950, and preferably the activity is increased non-targeted, whereby the respective line disclose in column 7 the fine chemical glucose.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in glucose compared with the wild type control.

The nucleic acid sequence of At3g62950 from Arabidopsis thaliana, e.g. as shown in the respective line in column 5 of Table I, application no. 17, has been published in F. And the activity of the gene product thereof is the activity of glutaredoxin.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said At3g62950, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g62950, or a functional equivalent or a homolog thereof as depicted in column 8 of Table II, application no. 17, preferably a homolog or functional equivalent as depicted in column 8 of Table II B, application no. 17, and being depicted in the same respective line as said At3g62950, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in glucose-1-phosphate compared with the wild type control.

The nucleic acid sequence of At3g62950 from Arabidopsis thaliana, e.g. as shown in the respective line in column 5 of Table I, application no. 17, has been published in F. And the activity of the gene product thereof is the activity of glutaredoxin.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said At3g62950, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g62950, or a functional equivalent or a homolog thereof as depicted in column 8 of Table II, application no. 17, preferably a homolog or functional equivalent as depicted in column 8 of Table II B, application no. 17, and being depicted in the same respective line as said At3g62950, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in myo-inositol compared with the wild type control.

The nucleic acid sequence of At3g62950 from Arabidopsis thaliana, e.g. as shown in the respective line in column 5 of Table I, application no. 17, has been published in F. And the activity of the gene product thereof is the activity of glutaredoxin.

Accordingly, in one embodiment, the process of the present invention for producing sedoheptulose-7-phosphate in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical sedoheptulose-7-phosphate), application no. 17, preferably the coding region thereof, or a homolog or a fragment thereof, and being depicted in the same respective line as said At3g62950, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g62950, or a functional equivalent or a homolog thereof as depicted in column 8 of Table II, application no. 17, preferably a homolog or functional equivalent as depicted in column 8 of Table II B, application no. 17, and being depicted in the same respective line as said At3g62950, and preferably the activity is increased non-targeted, whereby the respective line disclose in column 7 the fine chemical sedoheptulose-7-phosphate.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in sedoheptulose7-phosphate compared with the wild type control.

The nucleic acid sequence of At3g62950 from Arabidopsis thaliana, e.g. as shown in the respective line in column 5 of Table I, application no. 17, has been published in F. And the activity of the gene product thereof is the activity of glutaredoxin.

Accordingly, in one embodiment, the process of the present invention for producing sucrose in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said At3g62950, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At3g62950, or a functional equivalent or a homolog thereof as depicted in column 8 of Table II, application no. 17, preferably a homolog or functional equivalent as depicted in column 8 of Table II B, application no. 17, and being depicted in the same respective line as said At3g62950, and preferably the activity is increased non-targeted, whereby the respective line disclose in column 7 the fine chemical sucrose.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in sucrose compared with the wild type control.

The nucleic acid sequence of At4g09960 from Arabidopsis thaliana, e.g. as shown in the respective line in column 5 of Table I, application no. 17, has been published in F. And the activity of the gene product thereof is the activity of transcription factor.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said At4g09960, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g09960, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g09960, or a functional equivalent or a homolog thereof as depicted in column 8 of Table II, application no. 17, preferably a homolog or functional equivalent as depicted in column 8 of Table II B, application no. 17, and being depicted in the same respective line as said At4g09960, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 109717, preferably the coding region thereof, conferred the production of or the increase in glucose-1-phosphate compared with the wild type control.

The nucleic acid sequence of At4g15660 from Arabidopsis thaliana, e.g. as shown in the respective line in column 5 of Table I, application no. 17, has been published in F. And the activity of the gene product thereof is the activity of monothiol glutaredoxin.

Accordingly, in one embodiment, the process of the present invention for producing fructose in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said At4g15660, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g15660, or a functional equivalent or a homolog thereof as depicted in column 8 of Table II, application no. 17, preferably a homolog or functional equivalent as depicted in column 8 of Table II B, application no. 17, and being depicted in the same respective line as said At4g15660, and preferably the activity is increased non-targeted, whereby the respective line disclose in column 7 the fine chemical fructose.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in fructose compared with the wild type control.

The nucleic acid sequence of At4g15660 from Arabidopsis thaliana, e.g. as shown in the respective line in column 5 of Table I, application no. 17, has been published in F. And the activity of the gene product thereof is the activity of monothiol glutaredoxin.

Accordingly, in one embodiment, the process of the present invention for producing glucose in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said At4g15660, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g15660, or a functional equivalent or a homolog thereof as depicted in column 8 of Table II, application no. 17, preferably a homolog or functional equivalent as depicted in column 8 of Table II B, application no. 17, and being depicted in the same respective line as said At4g15660, and preferably the activity is increased non-targeted, whereby the respective line disclose in column 7 the fine chemical glucose.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in glucose compared with the wild type control.

The nucleic acid sequence of At4g15660 from Arabidopsis thaliana, e.g. as shown in the respective line in column 5 of Table I, application no. 17, has been published in F. And the activity of the gene product thereof is the activity of monothiol glutaredoxin.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said At4g15660, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g15660, or a functional equivalent or a homolog thereof as depicted in column 8 of Table II, application no. 17, preferably a homolog or functional equivalent as depicted in column 8 of Table II B, application no. 17, and being depicted in the same respective line as said At4g15660, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in glucose-1-phosphate compared with the wild type control.

The nucleic acid sequence of At4g15660 from Arabidopsis thaliana, e.g. as shown in the respective line in column 5 of Table I, application no. 17, has been published in F. And the activity of the gene product thereof is the activity of monothiol glutaredoxin.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said At4g15660, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g15660, or a functional equivalent or a homolog thereof as depicted in column 8 of Table II, application no. 17, preferably a homolog or functional equivalent as depicted in column 8 of Table II B, application no. 17, and being depicted in the same respective line as said At4g15660, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in myo-inositol compared with the wild type control.

The nucleic acid sequence of At4g15660 from Arabidopsis thaliana, e.g. as shown in the respective line in column 5 of Table I, application no. 17, has been published in F. And the activity of the gene product thereof is the activity of monothiol glutaredoxin.

Accordingly, in one embodiment, the process of the present invention for producing sedoheptulose-7-phosphate in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical sedoheptulose-7-phosphate), application no. 17, preferably the coding region thereof, or a homolog or a fragment thereof, and being depicted in the same respective line as said At4g15660, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g15660, or a functional equivalent or a homolog thereof as depicted in column 8 of Table II, application no. 17, preferably a homolog or functional equivalent as depicted in column 8 of Table II B, application no. 17, and being depicted in the same respective line as said At4g15660, and preferably the activity is increased non-targeted, whereby the respective line disclose in column 7 the fine chemical sedoheptulose-7-phosphate.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in sedoheptulose-7-phosphate compared with the wild type control.

The nucleic acid sequence of At4g15660 from Arabidopsis thaliana, e.g. as shown in the respective line in column 5 of Table I, application no. 17, has been published in F. And the activity of the gene product thereof is the activity of monothiol glutaredoxin.

Accordingly, in one embodiment, the process of the present invention for producing sucrose in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said At4g15660, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g15660, or a functional equivalent or a homolog thereof as depicted in column 8 of Table II, application no. 17, preferably a homolog or functional equivalent as depicted in column 8 of Table II B, application no. 17, and being depicted in the same respective line as said At4g15660, and preferably the activity is increased non-targeted, whereby the respective line disclose in column 7 the fine chemical sucrose.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in sucrose compared with the wild type control.

The nucleic acid sequence of At4g15670 from Arabidopsis thaliana, e.g. as shown in the respective line in column 5 of Table I, application no. 17, has been published in F. And the activity of the gene product thereof is the activity of monothiol glutaredoxin.

Accordingly, in one embodiment, the process of the present invention for producing fructose in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said At4g15670, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g15670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g15670, or a functional equivalent or a homolog thereof as depicted in column 8 of Table II, application no. 17, preferably a homolog or functional equivalent as depicted in column 8 of Table II B, application no. 17, and being depicted in the same respective line as said At4g15670, and preferably the activity is increased non-targeted, whereby the respective line disclose in column 7 the fine chemical fructose.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3279, preferably the coding region thereof, conferred the production of or the increase in fructose compared with the wild type control.

The nucleic acid sequence of At4g15670 from Arabidopsis thaliana, e.g. as shown in the respective line in column 5 of Table I, application no. 17, has been published in F. And the activity of the gene product thereof is the activity of monothiol glutaredoxin.

Accordingly, in one embodiment, the process of the present invention for producing glucose in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said At4g15670, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g15670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g15670, or a functional equivalent or a homolog thereof as depicted in column 8 of Table II, application no. 17, preferably a homolog or functional equivalent as depicted in column 8 of Table II B, application no. 17, and being depicted in the same respective line as said At4g15670, and preferably the activity is increased non-targeted, whereby the respective line disclose in column 7 the fine chemical glucose.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3279, preferably the coding region thereof, conferred the production of or the increase in glucose compared with the wild type control.

The nucleic acid sequence of At4g15670 from Arabidopsis thaliana, e.g. as shown in the respective line in column 5 of Table I, application no. 17, has been published in F. And the activity of the gene product thereof is the activity of monothiol glutaredoxin.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said At4g15670, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g15670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g15670, or a functional equivalent or a homolog thereof as depicted in column 8 of Table II, application no. 17, preferably a homolog or functional equivalent as depicted in column 8 of Table II B, application no. 17, and being depicted in the same respective line as said At4g15670, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3279, preferably the coding region thereof, conferred the production of or the increase in glucose-1-phosphate compared with the wild type control.

The nucleic acid sequence of At4g15670 from Arabidopsis thaliana, e.g. as shown in the respective line in column 5 of Table I, application no. 17, has been published in F. And the activity of the gene product thereof is the activity of monothiol glutaredoxin.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said At4g15670, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g15670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g15670, or a functional equivalent or a homolog thereof as depicted in column 8 of Table II, application no. 17, preferably a homolog or functional equivalent as depicted in column 8 of Table II B, application no. 17, and being depicted in the same respective line as said At4g15670, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3279, preferably the coding region thereof, conferred the production of or the increase in myo-inositol compared with the wild type control.

The nucleic acid sequence of At4g15670 from Arabidopsis thaliana, e.g. as shown in the respective line in column 5 of Table I, application no. 17, has been published in F. And the activity of the gene product thereof is the activity of monothiol glutaredoxin.

Accordingly, in one embodiment, the process of the present invention for producing sedoheptulose-7-phosphate in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical sedoheptulose-7-phosphate), application no. 17, preferably the coding region thereof, or a homolog or a fragment thereof, and being depicted in the same respective line as said At4g15670, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g15670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g15670, or a functional equivalent or a homolog thereof as depicted in column 8 of Table II, application no. 17, preferably a homolog or functional equivalent as depicted in column 8 of Table II B, application no. 17, and being depicted in the same respective line as said At4g15670, and preferably the activity is increased non-targeted, whereby the respective line disclose in column 7 the fine chemical sedoheptulose-7-phosphate.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3279, preferably the coding region thereof, conferred the production of or the increase in sedoheptulose-7-phosphate compared with the wild type control.

The nucleic acid sequence of At4g15670 from Arabidopsis thaliana, e.g. as shown in the respective line in column 5 of Table I, application no. 17, has been published in F. And the activity of the gene product thereof is the activity of monothiol glutaredoxin.

Accordingly, in one embodiment, the process of the present invention for producing sucrose in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said At4g15670, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g15670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g15670, or a functional equivalent or a homolog thereof as depicted in column 8 of Table II, application no. 17, preferably a homolog or functional equivalent as depicted in column 8 of Table II B, application no. 17, and being depicted in the same respective line as said At4g15670, and preferably the activity is increased non-targeted, whereby the respective line disclose in column 7 the fine chemical sucrose.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3279, preferably the coding region thereof, conferred the production of or the increase in sucrose compared with the wild type control.

The nucleic acid sequence of At4g15690 from Arabidopsis thaliana, e.g. as shown in the respective line in column 5 of Table I, application no. 17, has been published in F. And the activity of the gene product thereof is the activity of monothiol glutaredoxin.

Accordingly, in one embodiment, the process of the present invention for producing fructose in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said At4g15690, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g15690, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g15690, or a functional equivalent or a homolog thereof as depicted in column 8 of Table II, application no. 17, preferably a homolog or functional equivalent as depicted in column 8 of Table II B, application no. 17, and being depicted in the same respective line as said At4g15690, and preferably the activity is increased non-targeted, whereby the respective line disclose in column 7 the fine chemical fructose.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23844, preferably the coding region thereof, conferred the production of or the increase in fructose compared with the wild type control.

The nucleic acid sequence of At4g15690 from Arabidopsis thaliana, e.g. as shown in the respective line in column 5 of Table I, application no. 17, has been published in F. And the activity of the gene product thereof is the activity of monothiol glutaredoxin.

Accordingly, in one embodiment, the process of the present invention for producing glucose in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said At4g15690, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g15690, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g15690, or a functional equivalent or a homolog thereof as depicted in column 8 of Table II, application no. 17, preferably a homolog or functional equivalent as depicted in column 8 of Table II B, application no. 17, and being depicted in the same respective line as said At4g15690, and preferably the activity is increased non-targeted, whereby the respective line disclose in column 7 the fine chemical glucose.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23844, preferably the coding region thereof, conferred the production of or the increase in glucose compared with the wild type control.

The nucleic acid sequence of At4g15690 from Arabidopsis thaliana, e.g. as shown in the respective line in column 5 of Table I, application no. 17, has been published in F. And the activity of the gene product thereof is the activity of monothiol glutaredoxin.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said At4g15690, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g15690, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g15690, or a functional equivalent or a homolog thereof as depicted in column 8 of Table II, application no. 17, preferably a homolog or functional equivalent as depicted in column 8 of Table II B, application no. 17, and being depicted in the same respective line as said At4g15690, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23844, preferably the coding region thereof, conferred the production of or the increase in glucose-1-phosphate compared with the wild type control.

The nucleic acid sequence of At4g15690 from Arabidopsis thaliana, e.g. as shown in the respective line in column 5 of Table I, application no. 17, has been published in F. And the activity of the gene product thereof is the activity of monothiol glutaredoxin.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said At4g15690, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g15690, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g15690, or a functional equivalent or a homolog thereof as depicted in column 8 of Table II, application no. 17, preferably a homolog or functional equivalent as depicted in column 8 of Table II B, application no. 17, and being depicted in the same respective line as said At4g15690, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23844, preferably the coding region thereof, conferred the production of or the increase in myo-inositol compared with the wild type control.

The nucleic acid sequence of At4g15690 from Arabidopsis thaliana, e.g. as shown in the respective line in column 5 of Table I, application no. 17, has been published in F. And the activity of the gene product thereof is the activity of monothiol glutaredoxin.

Accordingly, in one embodiment, the process of the present invention for producing sucrose in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said At4g15690, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g15690, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g15690, or a functional equivalent or a homolog thereof as depicted in column 8 of Table II, application no. 17, preferably a homolog or functional equivalent as depicted in column 8 of Table II B, application no. 17, and being depicted in the same respective line as said At4g15690, and preferably the activity is increased non-targeted, whereby the respective line disclose in column 7 the fine chemical sucrose.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23844, preferably the coding region thereof, conferred the production of or the increase in sucrose compared with the wild type control.

The nucleic acid sequence of At4g15700 from Arabidopsis thaliana, e.g. as shown in the respective line in column 5 of Table I, application no. 17, has been published in F. And the activity of the gene product thereof is the activity of monothiol glutaredoxin.

Accordingly, in one embodiment, the process of the present invention for producing fructose in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said At4g15700, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g15700, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g15700, or a functional equivalent or a homolog thereof as depicted in column 8 of Table II, application no. 17, preferably a homolog or functional equivalent as depicted in column 8 of Table II B, application no. 17, and being depicted in the same respective line as said At4g15700, and preferably the activity is increased non-targeted, whereby the respective line disclose in column 7 the fine chemical fructose.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3654, preferably the coding region thereof, conferred the production of or the increase in fructose compared with the wild type control.

The nucleic acid sequence of At4g15700 from Arabidopsis thaliana, e.g. as shown in the respective line in column 5 of Table I, application no. 17, has been published in F. And the activity of the gene product thereof is the activity of monothiol glutaredoxin.

Accordingly, in one embodiment, the process of the present invention for producing glucose in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said At4g15700, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g15700, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g15700, or a functional equivalent or a homolog thereof as depicted in column 8 of Table II, application no. 17, preferably a homolog or functional equivalent as depicted in column 8 of Table II B, application no. 17, and being depicted in the same respective line as said At4g15700, and preferably the activity is increased non-targeted, whereby the respective line disclose in column 7 the fine chemical glucose.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3654, preferably the coding region thereof, conferred the production of or the increase in glucose compared with the wild type control.

The nucleic acid sequence of At4g15700 from Arabidopsis thaliana, e.g. as shown in the respective line in column 5 of Table I, application no. 17, has been published in F. And the activity of the gene product thereof is the activity of monothiol glutaredoxin.

Accordingly, in one embodiment, the process of the present invention for producing sedoheptulose-7-phosphate in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical sedoheptulose-7-phosphate), application no. 17, preferably the coding region thereof, or a homolog or a fragment thereof, and being depicted in the same respective line as said At4g15700, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g15700, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g15700, or a functional equivalent or a homolog thereof as depicted in column 8 of Table II, application no. 17, preferably a homolog or functional equivalent as depicted in column 8 of Table II B, application no. 17, and being depicted in the same respective line as said At4g15700, and preferably the activity is increased non-targeted, whereby the respective line disclose in column 7 the fine chemical sedoheptulose-7-phosphate.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3654, preferably the coding region thereof, conferred the production of or the increase in sedoheptulose-7-phosphate compared with the wild type control.

The nucleic acid sequence of At4g15700 from Arabidopsis thaliana, e.g. as shown in the respective line in column 5 of Table I, application no. 17, has been published in F. And the activity of the gene product thereof is the activity of monothiol glutaredoxin.

Accordingly, in one embodiment, the process of the present invention for producing sucrose in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said At4g15700, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g15700, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g15700, or a functional equivalent or a homolog thereof as depicted in column 8 of Table II, application no. 17, preferably a homolog or functional equivalent as depicted in column 8 of Table II B, application no. 17, and being depicted in the same respective line as said At4g15700, and preferably the activity is increased non-targeted, whereby the respective line disclose in column 7 the fine chemical sucrose.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3654, preferably the coding region thereof, conferred the production of or the increase in sucrose compared with the wild type control.

The nucleic acid sequence of At4g26080 from Arabidopsis thaliana, e.g. as shown in the respective line in column 5 of Table I, application no. 17, has been published in F. And the activity of the gene product thereof is the activity of protein phosphatase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said At4g26080, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g26080, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g26080, or a functional equivalent or a homolog thereof as depicted in column 8 of Table II, application no. 17, preferably a homolog or functional equivalent as depicted in column 8 of Table II B, application no. 17, and being depicted in the same respective line as said At4g26080, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 68849, preferably the coding region thereof, conferred the production of or the increase in myo-inositol compared with the wild type control.

The nucleic acid sequence of At4g33040 from Arabidopsis thaliana, e.g. as shown in the respective line in column 5 of Table I, application no. 17, has been published in F. And the activity of the gene product thereof is the activity of glutaredoxin.

Accordingly, in one embodiment, the process of the present invention for producing fructose in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said At4g33040, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g33040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g33040, or a functional equivalent or a homolog thereof as depicted in column 8 of Table II, application no. 17, preferably a homolog or functional equivalent as depicted in column 8 of Table II B, application no. 17, and being depicted in the same respective line as said At4g33040, and preferably the activity is increased non-targeted, whereby the respective line disclose in column 7 the fine chemical fructose.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 4102, preferably the coding region thereof, conferred the production of or the increase in fructose compared with the wild type control.

The nucleic acid sequence of At4g33040 from Arabidopsis thaliana, e.g. as shown in the respective line in column 5 of Table I, application no. 17, has been published in F. And the activity of the gene product thereof is the activity of glutaredoxin.

Accordingly, in one embodiment, the process of the present invention for producing glucose in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said At4g33040, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g33040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g33040, or a functional equivalent or a homolog thereof as depicted in column 8 of Table II, application no. 17, preferably a homolog or functional equivalent as depicted in column 8 of Table II B, application no. 17, and being depicted in the same respective line as said At4g33040, and preferably the activity is increased non-targeted, whereby the respective line disclose in column 7 the fine chemical glucose.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 4102, preferably the coding region thereof, conferred the production of or the increase in glucose compared with the wild type control.

The nucleic acid sequence of At4g33040 from Arabidopsis thaliana, e.g. as shown in the respective line in column 5 of Table I, application no. 17, has been published in F. And the activity of the gene product thereof is the activity of glutaredoxin.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said At4g33040, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g33040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g33040, or a functional equivalent or a homolog thereof as depicted in column 8 of Table II, application no. 17, preferably a homolog or functional equivalent as depicted in column 8 of Table II B, application no. 17, and being depicted in the same respective line as said At4g33040, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 4102, preferably the coding region thereof, conferred the production of or the increase in glucose-1-phosphate compared with the wild type control.

The nucleic acid sequence of At4g33040 from Arabidopsis thaliana, e.g. as shown in the respective line in column 5 of Table I, application no. 17, has been published in F. And the activity of the gene product thereof is the activity of glutaredoxin.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said At4g33040, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g33040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g33040, or a functional equivalent or a homolog thereof as depicted in column 8 of Table II, application no. 17, preferably a homolog or functional equivalent as depicted in column 8 of Table II B, application no. 17, and being depicted in the same respective line as said At4g33040, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 4102, preferably the coding region thereof, conferred the production of or the increase in myo-inositol compared with the wild type control.

The nucleic acid sequence of At4g33040 from Arabidopsis thaliana, e.g. as shown in the respective line in column 5 of Table I, application no. 17, has been published in F. And the activity of the gene product thereof is the activity of glutaredoxin.

Accordingly, in one embodiment, the process of the present invention for producing sedoheptulose-7-phosphate in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical sedoheptulose-7-phosphate), application no. 17, preferably the coding region thereof, or a homolog or a fragment thereof, and being depicted in the same respective line as said At4g33040, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g33040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g33040, or a functional equivalent or a homolog thereof as depicted in column 8 of Table II, application no. 17, preferably a homolog or functional equivalent as depicted in column 8 of Table II B, application no. 17, and being depicted in the same respective line as said At4g33040, and preferably the activity is increased non-targeted, whereby the respective line disclose in column 7 the fine chemical sedoheptulose-7-phosphate.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 4102, preferably the coding region thereof, conferred the production of or the increase in sedoheptulose7-phosphate compared with the wild type control.

The nucleic acid sequence of At4g33040 from Arabidopsis thaliana, e.g. as shown in the respective line in column 5 of Table I, application no. 17, has been published in F. And the activity of the gene product thereof is the activity of glutaredoxin.

Accordingly, in one embodiment, the process of the present invention for producing sucrose in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said At4g33040, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g33040, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g33040, or a functional equivalent or a homolog thereof as depicted in column 8 of Table II, application no. 17, preferably a homolog or functional equivalent as depicted in column 8 of Table II B, application no. 17, and being depicted in the same respective line as said At4g33040, and preferably the activity is increased non-targeted, whereby the respective line disclose in column 7 the fine chemical sucrose.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 4102, preferably the coding region thereof, conferred the production of or the increase in sucrose compared with the wild type control.

The nucleic acid sequence of At4g34160 from Arabidopsis thaliana, e.g. as shown in the respective line in column 5 of Table I, application no. 17, has been published in F. And the activity of the gene product thereof is the activity of cyclin D .

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “cyclin D”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said At4g34160, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g34160, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g34160, or a functional equivalent or a homolog thereof as depicted in column 8 of Table II, application no. 17, preferably a homolog or functional equivalent as depicted in column 8 of Table II B, application no. 17, and being depicted in the same respective line as said At4g34160, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 24311, preferably the coding region thereof, conferred the production of or the increase in glucose-1-phosphate compared with the wild type control.

The nucleic acid sequence of At4g34160 from Arabidopsis thaliana, e.g. as shown in the respective line in column 5 of Table I, application no. 17, has been published in F. And the activity of the gene product thereof is the activity of cyclin D .

Accordingly, in one embodiment, the process of the present invention for producing raffinose in a non-human organism, like a microorganism or a plant or a part thereof, comprises increasing or generating the activity of a gene product with the activity of a gene product conferring the activity of “cyclin D”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said At4g34160, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g34160, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g34160, or a functional equivalent or a homolog thereof as depicted in column 8 of Table II, application no. 17, preferably a homolog or functional equivalent as depicted in column 8 of Table II B, application no. 17, and being depicted in the same respective line as said At4g34160, and preferably the activity is increased non-targeted, whereby the respective line disclose in column 7 the fine chemical raffinose.

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 24311, preferably the coding region thereof, conferred the production of or the increase in raffinose compared with the wild type control.

The nucleic acid sequence of At4g34160 from Arabidopsis thaliana, e.g. as shown in the respective line in column 5 of Table I, application no. 17, has been published in F. And the activity of the gene product thereof is the activity of cyclin D .

Accordingly, in one embodiment, the process of the present invention for producing sucrose in a non-human organism, like a microorganism or a plant or a part thereof, comprises increasing or generating the activity of a gene product with the activity of a gene product conferring the activity of “cyclin D”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said At4g34160, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g34160, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g34160, or a functional equivalent or a homolog thereof as depicted in column 8 of Table II, application no. 17, preferably a homolog or functional equivalent as depicted in column 8 of Table II B, application no. 17, and being depicted in the same respective line as said At4g34160, and preferably the activity is increased non-targeted, whereby the respective line disclose in column 7 the fine chemical sucrose.

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 24311, preferably the coding region thereof, conferred the production of or the increase in sucrose compared with the wild type control.

The nucleic acid sequence of At4g35310 from Arabidopsis thaliana, e.g. as shown in the respective line in column 5 of Table I, application no. 17, has been published in F. And the activity of the gene product thereof is the activity of calcium-dependent protein kinase.

Accordingly, in one embodiment, the process of the present invention for producing fructose in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said At4g35310, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g35310, or a functional equivalent or a homolog thereof as depicted in column 8 of Table II, application no. 17, preferably a homolog or functional equivalent as depicted in column 8 of Table II B, application no. 17, and being depicted in the same respective line as said At4g35310, and preferably the activity is increased non-targeted, whereby the respective line disclose in column 7 the fine chemical fructose.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in fructose compared with the wild type control.

The nucleic acid sequence of At4g35310 from Arabidopsis thaliana, e.g. as shown in the respective line in column 5 of Table I, application no. 17, has been published in F. And the activity of the gene product thereof is the activity of calcium-dependent protein kinase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said At4g35310, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g35310, or a functional equivalent or a homolog thereof as depicted in column 8 of Table II, application no. 17, preferably a homolog or functional equivalent as depicted in column 8 of Table II B, application no. 17, and being depicted in the same respective line as said At4g35310, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in glucose-1-phosphate compared with the wild type control.

The nucleic acid sequence of At4g35310 from Arabidopsis thaliana, e.g. as shown in the respective line in column 5 of Table I, application no. 17, has been published in F. And the activity of the gene product thereof is the activity of calcium-dependent protein kinase.

Accordingly, in one embodiment, the process of the present invention for producing raffinose in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said At4g35310, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At4g35310, or a functional equivalent or a homolog thereof as depicted in column 8 of Table II, application no. 17, preferably a homolog or functional equivalent as depicted in column 8 of Table II B, application no. 17, and being depicted in the same respective line as said At4g35310, and preferably the activity is increased non-targeted, whereby the respective line disclose in column 7 the fine chemical raffinose.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in raffinose compared with the wild type control.

The nucleic acid sequence of At5g02760 from Arabidopsis thaliana, e.g. as shown in the respective line in column 5 of Table I, application no. 17, has been published in F. And the activity of the gene product thereof is the activity of protein phosphatase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said At5g02760, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g02760, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g02760, or a functional equivalent or a homolog thereof as depicted in column 8 of Table II, application no. 17, preferably a homolog or functional equivalent as depicted in column 8 of Table II B, application no. 17, and being depicted in the same respective line as said At5g02760, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 68987, preferably the coding region thereof, conferred the production of or the increase in myo-inositol compared with the wild type control.

The nucleic acid sequence of At5g03720 from Arabidopsis thaliana, e.g. as shown in the respective line in column 5 of Table I, application no. 17, has been published in F. And the activity of the gene product thereof is the activity of heat shock transcription factor.

Accordingly, in one embodiment, the process of the present invention for producing fructose in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said At5g03720, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g03720, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g03720, or a functional equivalent or a homolog thereof as depicted in column 8 of Table II, application no. 17, preferably a homolog or functional equivalent as depicted in column 8 of Table II B, application no. 17, and being depicted in the same respective line as said At5g03720, and preferably the activity is increased non-targeted, whereby the respective line disclose in column 7 the fine chemical fructose.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 24438, preferably the coding region thereof, conferred the production of or the increase in fructose compared with the wild type control.

The nucleic acid sequence of At5g03720 from Arabidopsis thaliana, e.g. as shown in the respective line in column 5 of Table I, application no. 17, has been published in F. And the activity of the gene product thereof is the activity of heat shock transcription factor.

Accordingly, in one embodiment, the process of the present invention for producing glucose in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said At5g03720, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g03720, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g03720, or a functional equivalent or a homolog thereof as depicted in column 8 of Table II, application no. 17, preferably a homolog or functional equivalent as depicted in column 8 of Table II B, application no. 17, and being depicted in the same respective line as said At5g03720, and preferably the activity is increased non-targeted, whereby the respective line disclose in column 7 the fine chemical glucose.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 24438, preferably the coding region thereof, conferred the production of or the increase in glucose compared with the wild type control.

The nucleic acid sequence of At5g03720 from Arabidopsis thaliana, e.g. as shown in the respective line in column 5 of Table I, application no. 17, has been published in F. And the activity of the gene product thereof is the activity of heat shock transcription factor.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said At5g03720, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g03720, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g03720, or a functional equivalent or a homolog thereof as depicted in column 8 of Table II, application no. 17, preferably a homolog or functional equivalent as depicted in column 8 of Table II B, application no. 17, and being depicted in the same respective line as said At5g03720, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 24438, preferably the coding region thereof, conferred the production of or the increase in glucose-1-phosphate compared with the wild type control.

The nucleic acid sequence of At5g03720 from Arabidopsis thaliana, e.g. as shown in the respective line in column 5 of Table I, application no. 17, has been published in F. And the activity of the gene product thereof is the activity of heat shock transcription factor.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said At5g03720, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g03720, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g03720, or a functional equivalent or a homolog thereof as depicted in column 8 of Table II, application no. 17, preferably a homolog or functional equivalent as depicted in column 8 of Table II B, application no. 17, and being depicted in the same respective line as said At5g03720, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 24438, preferably the coding region thereof, conferred the production of or the increase in myo-inositol compared with the wild type control.

The nucleic acid sequence of At5g03720 from Arabidopsis thaliana, e.g. as shown in the respective line in column 5 of Table I, application no. 17, has been published in F. And the activity of the gene product thereof is the activity of heat shock transcription factor.

Accordingly, in one embodiment, the process of the present invention for producing raffinose in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said At5g03720, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g03720, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g03720, or a functional equivalent or a homolog thereof as depicted in column 8 of Table II, application no. 17, preferably a homolog or functional equivalent as depicted in column 8 of Table II B, application no. 17, and being depicted in the same respective line as said At5g03720, and preferably the activity is increased non-targeted, whereby the respective line disclose in column 7 the fine chemical raffinose.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 24438, preferably the coding region thereof, conferred the production of or the increase in raffinose compared with the wild type control.

The nucleic acid sequence of At5g03720 from Arabidopsis thaliana, e.g. as shown in the respective line in column 5 of Table I, application no. 17, has been published in F. And the activity of the gene product thereof is the activity of heat shock transcription factor.

Accordingly, in one embodiment, the process of the present invention for producing sucrose in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said At5g03720, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g03720, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g03720, or a functional equivalent or a homolog thereof as depicted in column 8 of Table II, application no. 17, preferably a homolog or functional equivalent as depicted in column 8 of Table II B, application no. 17, and being depicted in the same respective line as said At5g03720, and preferably the activity is increased non-targeted, whereby the respective line disclose in column 7 the fine chemical sucrose.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 24438, preferably the coding region thereof, conferred the production of or the increase in sucrose compared with the wild type control.

The nucleic acid sequence of At5g07200 from Arabidopsis thaliana, e.g. as shown in the respective line in column 5 of Table I, application no. 17, has been published in F. And the activity of the gene product thereof is the activity of gibberellin 20-oxidase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “gibberellin 20-oxidase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said At5g07200, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g07200, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g07200, or a functional equivalent or a homolog thereof as depicted in column 8 of Table II, application no. 17, preferably a homolog or functional equivalent as depicted in column 8 of Table II B, application no. 17, and being depicted in the same respective line as said At5g07200, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “gibberellin 20-oxidase”, preferably it is the molecule of section (a) or (b) of this paragraph.

In particular, it was observed that in plants, especially in Arabidopsis thaliana, increasing or generating the activity of a gene product non-targeted with the activity of a “gibberellin 20-oxidase”, preferably being encoded by a gene comprising the nucleic acid sequence SEQ ID NO. 24492, preferably the coding region thereof, conferred the production of or the increase in myo-inositol compared with the wild type control.

The nucleic acid sequence of At5g10170 from Arabidopsis thaliana, e.g. as shown in the respective line in column 5 of Table I, application no. 17, has been published in F. And the activity of the gene product thereof is the activity of inositol-3-phosphate synthase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “inositol-3-phosphate synthase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said At5g10170, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g10170, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g10170, or a functional equivalent or a homolog thereof as depicted in column 8 of Table II, application no. 17, preferably a homolog or functional equivalent as depicted in column 8 of Table II B, application no. 17, and being depicted in the same respective line as said At5g10170, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “inositol-3-phosphate synthase”, preferably it is the molecule of section (a) or (b) of this paragraph.

In particular, it was observed that in plants, especially in Arabidopsis thaliana, increasing or generating the activity of a gene product non-targeted with the activity of a “inositol-3-phosphate synthase”, preferably being encoded by a gene comprising the nucleic acid sequence SEQ ID NO. 142753, preferably the coding region thereof, conferred the production of or the increase in myo-inositol compared with the wild type control.

The nucleic acid sequence of At5g10170 from Arabidopsis thaliana, e.g. as shown in the respective line in column 5 of Table I, application no. 17, has been published in F. And the activity of the gene product thereof is the activity of inositol-3-phosphate synthase.

Accordingly, in one embodiment, the process of the present invention for producing raffinose in a non-human organism, like a microorganism or a plant or a part thereof, comprises increasing or generating the activity of a gene product with the activity of a gene product conferring the activity of “inositol-3-phosphate synthase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said At5g10170, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g10170, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g10170, or a functional equivalent or a homolog thereof as depicted in column 8 of Table II, application no. 17, preferably a homolog or functional equivalent as depicted in column 8 of Table II B, application no. 17, and being depicted in the same respective line as said At5g10170, and preferably the activity is increased non-targeted, whereby the respective line disclose in column 7 the fine chemical raffinose.

Accordingly, in one embodiment, the molecule which activity is to be increased in the process of the invention is the gene product with an activity as a “inositol-3-phosphate synthase”, preferably it is the molecule of section (a) or (b) of this paragraph.

In particular, it was observed that in plants, especially in Arabidopsis thaliana, increasing or generating the activity of a gene product non-targeted with the activity of a “inositol-3-phosphate synthase”, preferably being encoded by a gene comprising the nucleic acid sequence SEQ ID NO. 142753, preferably the coding region thereof, conferred the production of or the increase in raffinose compared with the wild type control.

The nucleic acid sequence of At5g14070 from Arabidopsis thaliana, e.g. as shown in the respective line in column 5 of Table I, application no. 17, has been published in F. And the activity of the gene product thereof is the activity of glutaredoxin.

Accordingly, in one embodiment, the process of the present invention for producing glucose in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said At5g14070, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g14070, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g14070, or a functional equivalent or a homolog thereof as depicted in column 8 of Table II, application no. 17, preferably a homolog or functional equivalent as depicted in column 8 of Table II B, application no. 17, and being depicted in the same respective line as said At5g14070, and preferably the activity is increased non-targeted, whereby the respective line disclose in column 7 the fine chemical glucose.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 69275, preferably the coding region thereof, conferred the production of or the increase in glucose compared with the wild type control.

The nucleic acid sequence of At5g16650 from Arabidopsis thaliana, e.g. as shown in the respective line in column 5 of Table I, application no. 17, has been published in F. And the activity of the gene product thereof is the activity of At5g16650-protein.

Accordingly, in one embodiment, the process of the present invention for producing fructose in a non-human organism, like a microorganism or a plant or a part thereof, comprises increasing or generating the activity of a gene product with the activity of a gene product conferring the activity of “At5g16650-protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said At5g16650, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g16650, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g16650, or a functional equivalent or a homolog thereof as depicted in column 8 of Table II, application no. 17, preferably a homolog or functional equivalent as depicted in column 8 of Table II B, application no. 17, and being depicted in the same respective line as said At5g16650, and preferably the activity is increased non-targeted, whereby the respective line disclose in column 7 the fine chemical fructose.

Accordingly, in one embodiment, the molecule which activity is to be increased in the process of the invention is the gene product with an activity as a “At5g16650-protein”, preferably it is the molecule of section (a) or (b) of this paragraph.

In particular, it was observed that in plants, especially in Arabidopsis thaliana, increasing or generating the activity of a gene product non-targeted with the activity of a “At5g16650-protein”, preferably being encoded by a gene comprising the nucleic acid sequence SEQ ID NO. 25283, preferably the coding region thereof, conferred the production of or the increase in fructose compared with the wild type control.

The nucleic acid sequence of At5g18600 from Arabidopsis thaliana, e.g. as shown in the respective line in column 5 of Table I, application no. 17, has been published in F. And the activity of the gene product thereof is the activity of monothiol glutaredoxin.

Accordingly, in one embodiment, the process of the present invention for producing fructose in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said At5g18600, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g18600, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g18600, or a functional equivalent or a homolog thereof as depicted in column 8 of Table II, application no. 17, preferably a homolog or functional equivalent as depicted in column 8 of Table II B, application no. 17, and being depicted in the same respective line as said At5g18600, and preferably the activity is increased non-targeted, whereby the respective line disclose in column 7 the fine chemical fructose.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 4904, preferably the coding region thereof, conferred the production of or the increase in fructose compared with the wild type control.

The nucleic acid sequence of At5g18600 from Arabidopsis thaliana, e.g. as shown in the respective line in column 5 of Table I, application no. 17, has been published in F. And the activity of the gene product thereof is the activity of monothiol glutaredoxin.

Accordingly, in one embodiment, the process of the present invention for producing glucose in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said At5g18600, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g18600, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g18600, or a functional equivalent or a homolog thereof as depicted in column 8 of Table II, application no. 17, preferably a homolog or functional equivalent as depicted in column 8 of Table II B, application no. 17, and being depicted in the same respective line as said At5g18600, and preferably the activity is increased non-targeted, whereby the respective line disclose in column 7 the fine chemical glucose.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 4904, preferably the coding region thereof, conferred the production of or the increase in glucose compared with the wild type control.

The nucleic acid sequence of At5g18600 from Arabidopsis thaliana, e.g. as shown in the respective line in column 5 of Table I, application no. 17, has been published in F. And the activity of the gene product thereof is the activity of monothiol glutaredoxin.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said At5g18600, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g18600, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g18600, or a functional equivalent or a homolog thereof as depicted in column 8 of Table II, application no. 17, preferably a homolog or functional equivalent as depicted in column 8 of Table II B, application no. 17, and being depicted in the same respective line as said At5g18600, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 4904, preferably the coding region thereof, conferred the production of or the increase in myo-inositol compared with the wild type control.

The nucleic acid sequence of At5g18600 from Arabidopsis thaliana, e.g. as shown in the respective line in column 5 of Table I, application no. 17, has been published in F. And the activity of the gene product thereof is the activity of monothiol glutaredoxin.

Accordingly, in one embodiment, the process of the present invention for producing sedoheptulose-7-phosphate in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical sedoheptulose-7-phosphate), application no. 17, preferably the coding region thereof, or a homolog or a fragment thereof, and being depicted in the same respective line as said At5g18600, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g18600, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g18600, or a functional equivalent or a homolog thereof as depicted in column 8 of Table II, application no. 17, preferably a homolog or functional equivalent as depicted in column 8 of Table II B, application no. 17, and being depicted in the same respective line as said At5g18600, and preferably the activity is increased non-targeted, whereby the respective line disclose in column 7 the fine chemical sedoheptulose-7-phosphate.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 4904, preferably the coding region thereof, conferred the production of or the increase in sedoheptulose-7-phosphate compared with the wild type control.

The nucleic acid sequence of At5g18600 from Arabidopsis thaliana, e.g. as shown in the respective line in column 5 of Table I, application no. 17, has been published in F. And the activity of the gene product thereof is the activity of monothiol glutaredoxin.

Accordingly, in one embodiment, the process of the present invention for producing sucrose in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said At5g18600, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g18600, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g18600, or a functional equivalent or a homolog thereof as depicted in column 8 of Table II, application no. 17, preferably a homolog or functional equivalent as depicted in column 8 of Table II B, application no. 17, and being depicted in the same respective line as said At5g18600, and preferably the activity is increased non-targeted, whereby the respective line disclose in column 7 the fine chemical sucrose.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 4904, preferably the coding region thereof, conferred the production of or the increase in sucrose compared with the wild type control.

The nucleic acid sequence of At5g39760 from Arabidopsis thaliana, e.g. as shown in the respective line in column 5 of Table I, application no. 17, has been published in F. And the activity of the gene product thereof is the activity of transcription factor.

Accordingly, in one embodiment, the process of the present invention for producing fructose in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said At5g39760, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g39760, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g39760, or a functional equivalent or a homolog thereof as depicted in column 8 of Table II, application no. 17, preferably a homolog or functional equivalent as depicted in column 8 of Table II B, application no. 17, and being depicted in the same respective line as said At5g39760, and preferably the activity is increased non-targeted, whereby the respective line disclose in column 7 the fine chemical fructose.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 25428, preferably the coding region thereof, conferred the production of or the increase in fructose compared with the wild type control.

The nucleic acid sequence of At5g43630 from Arabidopsis thaliana, e.g. as shown in the respective line in column 5 of Table I, application no. 17, has been published in F. And the activity of the gene product thereof is the activity of At5g43630-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “At5g43630-protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said At5g43630, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g43630, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g43630, or a functional equivalent or a homolog thereof as depicted in column 8 of Table II, application no. 17, preferably a homolog or functional equivalent as depicted in column 8 of Table II B, application no. 17, and being depicted in the same respective line as said At5g43630, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “At5g43630-protein”, preferably it is the molecule of section (a) or (b) of this paragraph.

In particular, it was observed that in plants, especially in Arabidopsis thaliana, increasing or generating the activity of a gene product non-targeted with the activity of a “At5g43630-protein”, preferably being encoded by a gene comprising the nucleic acid sequence SEQ ID NO. 142929, preferably the coding region thereof, conferred the production of or the increase in glucose-1-phosphate compared with the wild type control.

The nucleic acid sequence of At5g54070 from Arabidopsis thaliana, e.g. as shown in the respective line in column 5 of Table I, application no. 17, has been published in F. And the activity of the gene product thereof is the activity of heat shock transcription factor.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said At5g54070, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g54070, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g54070, or a functional equivalent or a homolog thereof as depicted in column 8 of Table II, application no. 17, preferably a homolog or functional equivalent as depicted in column 8 of Table II B, application no. 17, and being depicted in the same respective line as said At5g54070, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 142933, preferably the coding region thereof, conferred the production of or the increase in myo-inositol compared with the wild type control.

The nucleic acid sequence of At5g54070 from Arabidopsis thaliana, e.g. as shown in the respective line in column 5 of Table I, application no. 17, has been published in F. And the activity of the gene product thereof is the activity of heat shock transcription factor.

Accordingly, in one embodiment, the process of the present invention for producing raffinose in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said At5g54070, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g54070, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g54070, or a functional equivalent or a homolog thereof as depicted in column 8 of Table II, application no. 17, preferably a homolog or functional equivalent as depicted in column 8 of Table II B, application no. 17, and being depicted in the same respective line as said At5g54070, and preferably the activity is increased non-targeted, whereby the respective line disclose in column 7 the fine chemical raffinose.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 142933, preferably the coding region thereof, conferred the production of or the increase in raffinose compared with the wild type control.

The nucleic acid sequence of At5g57050 from Arabidopsis thaliana, e.g. as shown in the respective line in column 5 of Table I, application no. 17, has been published in F. And the activity of the gene product thereof is the activity of protein phosphatase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said At5g57050, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g57050, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g57050, or a functional equivalent or a homolog thereof as depicted in column 8 of Table II, application no. 17, preferably a homolog or functional equivalent as depicted in column 8 of Table II B, application no. 17, and being depicted in the same respective line as said At5g57050, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 5318, preferably the coding region thereof, conferred the production of or the increase in glucose-1-phosphate compared with the wild type control.

The nucleic acid sequence of At5g57050 from Arabidopsis thaliana, e.g. as shown in the respective line in column 5 of Table I, application no. 17, has been published in F. And the activity of the gene product thereof is the activity of protein phosphatase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said At5g57050, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g57050, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g57050, or a functional equivalent or a homolog thereof as depicted in column 8 of Table II, application no. 17, preferably a homolog or functional equivalent as depicted in column 8 of Table II B, application no. 17, and being depicted in the same respective line as said At5g57050, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 5318, preferably the coding region thereof, conferred the production of or the increase in myo-inositol compared with the wild type control.

The nucleic acid sequence of At5g60110 from Arabidopsis thaliana, e.g. as shown in the respective line in column 5 of Table I, application no. 17, has been published in F. And the activity of the gene product thereof is the activity of RNA-binding protein.

Accordingly, in one embodiment, the process of the present invention for producing fructose in a non-human organism, like a microorganism or a plant or a part thereof, comprises increasing or generating the activity of a gene product with the activity of a gene product conferring the activity of “RNA-binding protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said At5g60110, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g60110, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g60110, or a functional equivalent or a homolog thereof as depicted in column 8 of Table II, application no. 17, preferably a homolog or functional equivalent as depicted in column 8 of Table II B, application no. 17, and being depicted in the same respective line as said At5g60110, and preferably the activity is increased non-targeted, whereby the respective line disclose in column 7 the fine chemical fructose.

Accordingly, in one embodiment, the molecule which activity is to be increased in the process of the invention is the gene product with an activity as a “RNA-binding protein”, preferably it is the molecule of section (a) or (b) of this paragraph.

In particular, it was observed that in plants, especially in Arabidopsis thaliana, increasing or generating the activity of a gene product non-targeted with the activity of a “RNA-binding protein”, preferably being encoded by a gene comprising the nucleic acid sequence SEQ ID NO. 70006, preferably the coding region thereof, conferred the production of or the increase in fructose compared with the wild type control.

The nucleic acid sequence of At5g60110 from Arabidopsis thaliana, e.g. as shown in the respective line in column 5 of Table I, application no. 17, has been published in F. And the activity of the gene product thereof is the activity of RNA-binding protein.

Accordingly, in one embodiment, the process of the present invention for producing glucose in a non-human organism, like a microorganism or a plant or a part thereof, comprises increasing or generating the activity of a gene product with the activity of a gene product conferring the activity of “RNA-binding protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said At5g60110, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g60110, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g60110, or a functional equivalent or a homolog thereof as depicted in column 8 of Table II, application no. 17, preferably a homolog or functional equivalent as depicted in column 8 of Table II B, application no. 17, and being depicted in the same respective line as said At5g60110, and preferably the activity is increased non-targeted, whereby the respective line disclose in column 7 the fine chemical glucose.

Accordingly, in one embodiment, the molecule which activity is to be increased in the process of the invention is the gene product with an activity as a “RNA-binding protein”, preferably it is the molecule of section (a) or (b) of this paragraph.

In particular, it was observed that in plants, especially in Arabidopsis thaliana, increasing or generating the activity of a gene product non-targeted with the activity of a “RNA-binding protein”, preferably being encoded by a gene comprising the nucleic acid sequence SEQ ID NO. 70006, preferably the coding region thereof, conferred the production of or the increase in glucose compared with the wild type control.

The nucleic acid sequence of At5g60440 from Arabidopsis thaliana, e.g. as shown in the respective line in column 5 of Table I, application no. 17, has been published in F. And the activity of the gene product thereof is the activity of MADS-box transcription factor.

Accordingly, in one embodiment, the process of the present invention for producing fructose in a non-human organism, like a microorganism or a plant or a part thereof, comprises increasing or generating the activity of a gene product with the activity of a gene product conferring the activity of “MADS-box transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said At5g60440, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g60440, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g60440, or a functional equivalent or a homolog thereof as depicted in column 8 of Table II, application no. 17, preferably a homolog or functional equivalent as depicted in column 8 of Table II B, application no. 17, and being depicted in the same respective line as said At5g60440, and preferably the activity is increased non-targeted, whereby the respective line disclose in column 7 the fine chemical fructose.

Accordingly, in one embodiment, the molecule which activity is to be increased in the process of the invention is the gene product with an activity as a “MADS-box transcription factor”, preferably it is the molecule of section (a) or (b) of this paragraph.

In particular, it was observed that in plants, especially in Arabidopsis thaliana, increasing or generating the activity of a gene product non-targeted with the activity of a “MADS-box transcription factor”, preferably being encoded by a gene comprising the nucleic acid sequence SEQ ID NO. 102899, preferably the coding region thereof, conferred the production of or the increase in fructose compared with the wild type control.

The nucleic acid sequence of At5g60440 from Arabidopsis thaliana, e.g. as shown in the respective line in column 5 of Table I, application no. 17, has been published in F. And the activity of the gene product thereof is the activity of MADS-box transcription factor.

Accordingly, in one embodiment, the process of the present invention for producing raffinose in a non-human organism, like a microorganism or a plant or a part thereof, comprises increasing or generating the activity of a gene product with the activity of a gene product conferring the activity of “MADS-box transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said At5g60440, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g60440, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g60440, or a functional equivalent or a homolog thereof as depicted in column 8 of Table II, application no. 17, preferably a homolog or functional equivalent as depicted in column 8 of Table II B, application no. 17, and being depicted in the same respective line as said At5g60440, and preferably the activity is increased non-targeted, whereby the respective line disclose in column 7 the fine chemical raffinose.

Accordingly, in one embodiment, the molecule which activity is to be increased in the process of the invention is the gene product with an activity as a “MADS-box transcription factor”, preferably it is the molecule of section (a) or (b) of this paragraph.

In particular, it was observed that in plants, especially in Arabidopsis thaliana, increasing or generating the activity of a gene product non-targeted with the activity of a “MADS-box transcription factor”, preferably being encoded by a gene comprising the nucleic acid sequence SEQ ID NO. 102899, preferably the coding region thereof, conferred the production of or the increase in raffinose compared with the wild type control.

The nucleic acid sequence of At5g60440 from Arabidopsis thaliana, e.g. as shown in the respective line in column 5 of Table I, application no. 17, has been published in F. And the activity of the gene product thereof is the activity of MADS-box transcription factor.

Accordingly, in one embodiment, the process of the present invention for producing saccharic acid in a non-human organism, like a microorganism or a plant or a part thereof, comprises increasing or generating the activity of a gene product with the activity of a gene product conferring the activity of “MADS-box transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical saccharic acid), application no. 17, preferably the coding region thereof, or a homolog or a fragment thereof, and being depicted in the same respective line as said At5g60440, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g60440, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g60440, or a functional equivalent or a homolog thereof as depicted in column 8 of Table II, application no. 17, preferably a homolog or functional equivalent as depicted in column 8 of Table II B, application no. 17, and being depicted in the same respective line as said At5g60440, and preferably the activity is increased non-targeted, whereby the respective line disclose in column 7 the fine chemical saccharic acid.

Accordingly, in one embodiment, the molecule which activity is to be increased in the process of the invention is the gene product with an activity as a “MADS-box transcription factor”, preferably it is the molecule of section (a) or (b) of this paragraph.

In particular, it was observed that in plants, especially in Arabidopsis thaliana, increasing or generating the activity of a gene product non-targeted with the activity of a “MADS-box transcription factor”, preferably being encoded by a gene comprising the nucleic acid sequence SEQ ID NO. 102899, preferably the coding region thereof, conferred the production of or the increase in saccharic acid compared with the wild type control.

The nucleic acid sequence of At5g66710 from Arabidopsis thaliana, e.g. as shown in the respective line in column 5 of Table I, application no. 17, has been published in F. And the activity of the gene product thereof is the activity of protein kinase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said At5g66710, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said At5g66710, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said At5g66710, or a functional equivalent or a homolog thereof as depicted in column 8 of Table II, application no. 17, preferably a homolog or functional equivalent as depicted in column 8 of Table II B, application no. 17, and being depicted in the same respective line as said At5g66710, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 122803, preferably the coding region thereof, conferred the production of or the increase in glucose-1-phosphate compared with the wild type control.

The nucleic acid sequence of AvinDRAFT_{—}1534 from Azotobacter vinelandii, e.g. as shown in the respective line in column 5 of Table I, application no. 17, has been published in D. And the activity of the gene product thereof is the activity of glycosyl transferase.

Accordingly, in one embodiment, the process of the present invention for producing raffinose in a non-human organism, like a microorganism or a plant or a part thereof, comprises increasing or generating the activity of a gene product with the activity of a gene product conferring the activity of “glycosyl transferase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said AvinDRAFT_{—}1534, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}1534, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}1534, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}1534, and preferably the activity is increased non-targeted, whereby the respective line disclose in column 7 the fine chemical raffinose.

Accordingly, in one embodiment, the molecule which activity is to be increased in the process of the invention is the gene product with an activity as a “glycosyl transferase”, preferably it is the molecule of section (a) or (b) of this paragraph.

In particular, it was observed that in plants, especially in Arabidopsis thaliana, increasing or generating the activity of a gene product non-targeted with the activity of a “glycosyl transferase”, preferably being encoded by a gene comprising the nucleic acid sequence SEQ ID NO. 26120, preferably the coding region thereof, conferred the production of or the increase in raffinose compared with the wild type control.

The nucleic acid sequence of AvinDRAFT_{—}1624 from Azotobacter vinelandii, e.g. as shown in the respective line in column 5 of Table I, application no. 17, has been published in D. And the activity of the gene product thereof is the activity of sec-independent protein translocase.

Accordingly, in one embodiment, the process of the present invention for producing sucrose in a non-human organism, like a microorganism or a plant or a part thereof, comprises increasing or generating the activity of a gene product with the activity of a gene product conferring the activity of “sec-independent protein translocase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said AvinDRAFT_{—}1624, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}1624, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}1624, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}1624, and preferably the activity is increased non-targeted, whereby the respective line disclose in column 7 the fine chemical sucrose.

Accordingly, in one embodiment, the molecule which activity is to be increased in the process of the invention is the gene product with an activity as a “sec-independent protein translocase”, preferably it is the molecule of section (a) or (b) of this paragraph.

In particular, it was observed that in plants, especially in Arabidopsis thaliana, increasing or generating the activity of a gene product non-targeted with the activity of a “sec-independent protein translocase”, preferably being encoded by a gene comprising the nucleic acid sequence SEQ ID NO. 26196, preferably the coding region thereof, conferred the production of or the increase in sucrose compared with the wild type control.

The nucleic acid sequence of AvinDRAFT_{—}2091 from Azotobacter vinelandii, e.g. as shown in the respective line in column 5 of Table I, application no. 17, has been published in D. And the activity of the gene product thereof is the activity of beta-hydroxylase.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “beta-hydroxylase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said AvinDRAFT_{—}2091, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}2091, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}2091, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}2091, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “beta-hydroxylase”, preferably it is the molecule of section (a) or (b) of this paragraph.

In particular, it was observed that in plants, especially in Arabidopsis thaliana, increasing or generating the activity of a gene product non-targeted with the activity of a “beta-hydroxylase”, preferably being encoded by a gene comprising the nucleic acid sequence SEQ ID NO. 6040, preferably the coding region thereof, conferred the production of or the increase in 3,4,5-trihydroxypentanoic acid compared with the wild type control.

The nucleic acid sequence of AvinDRAFT_{—}2344 from Azotobacter vinelandii, e.g. as shown in the respective line in column 5 of Table I, application no. 17, has been published in D. And the activity of the gene product thereof is the activity of elongation factor Tu.

Accordingly, in one embodiment, the process of the present invention for producing fructose in a non-human organism, like a microorganism or a plant or a part thereof, comprises increasing or generating the activity of a gene product with the activity of a gene product conferring the activity of “elongation factor Tu”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said AvinDRAFT_{—}2344, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}2344, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}2344, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}2344, and preferably the activity is increased non-targeted, whereby the respective line disclose in column 7 the fine chemical fructose.

Accordingly, in one embodiment, the molecule which activity is to be increased in the process of the invention is the gene product with an activity as a “elongation factor Tu”, preferably it is the molecule of section (a) or (b) of this paragraph.

In particular, it was observed that in plants, especially in Arabidopsis thaliana, increasing or generating the activity of a gene product non-targeted with the activity of a “elongation factor Tu”, preferably being encoded by a gene comprising the nucleic acid sequence SEQ ID NO. 27021, preferably the coding region thereof, conferred the production of or the increase in fructose compared with the wild type control.

The nucleic acid sequence of AvinDRAFT_{—}2344 from Azotobacter vinelandii, e.g. as shown in the respective line in column 5 of Table I, application no. 17, has been published in D. And the activity of the gene product thereof is the activity of elongation factor Tu.

Accordingly, in one embodiment, the process of the present invention for producing glucose in a non-human organism, like a microorganism or a plant or a part thereof, comprises increasing or generating the activity of a gene product with the activity of a gene product conferring the activity of “elongation factor Tu”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said AvinDRAFT_{—}2344, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}2344, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}2344, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}2344, and preferably the activity is increased non-targeted, whereby the respective line disclose in column 7 the fine chemical glucose.

Accordingly, in one embodiment, the molecule which activity is to be increased in the process of the invention is the gene product with an activity as a “elongation factor Tu”, preferably it is the molecule of section (a) or (b) of this paragraph.

In particular, it was observed that in plants, especially in Arabidopsis thaliana, increasing or generating the activity of a gene product non-targeted with the activity of a “elongation factor Tu”, preferably being encoded by a gene comprising the nucleic acid sequence SEQ ID NO. 27021, preferably the coding region thereof, conferred the production of or the increase in glucose compared with the wild type control.

The nucleic acid sequence of AvinDRAFT_{—}2521 from Azotobacter vinelandii, e.g. as shown in the respective line in column 5 of Table I, application no. 17, has been published in D. And the activity of the gene product thereof is the activity of ABC transporter permease protein.

Accordingly, in one embodiment, the process of the present invention for producing fructose in a non-human organism, like a microorganism or a plant or a part thereof, comprises increasing or generating the activity of a gene product with the activity of a gene product conferring the activity of “ABC transporter permease protein”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said AvinDRAFT_{—}2521, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}2521, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}2521, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}2521, and preferably the activity is increased non-targeted, whereby the respective line disclose in column 7 the fine chemical fructose.

Accordingly, in one embodiment, the molecule which activity is to be increased in the process of the invention is the gene product with an activity as a “ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “ABC transporter permease protein”, preferably being encoded by a gene comprising the nucleic acid sequence SEQ ID NO. 27882, preferably the coding region thereof, conferred the production of or the increase in fructose compared with the wild type control.

The nucleic acid sequence of AvinDRAFT_{—}2521 from Azotobacter vinelandii, e.g. as shown in the respective line in column 5 of Table I, application no. 17, has been published in D. And the activity of the gene product thereof is the activity of ABC transporter permease protein.

Accordingly, in one embodiment, the process of the present invention for producing sucrose in a non-human organism, like a microorganism or a plant or a part thereof, comprises increasing or generating the activity of a gene product with the activity of a gene product conferring the activity of “ABC transporter permease protein”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said AvinDRAFT_{—}2521, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}2521, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}2521, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}2521, and preferably the activity is increased non-targeted, whereby the respective line disclose in column 7 the fine chemical sucrose.

Accordingly, in one embodiment, the molecule which activity is to be increased in the process of the invention is the gene product with an activity as a “ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “ABC transporter permease protein”, preferably being encoded by a gene comprising the nucleic acid sequence SEQ ID NO. 27882, preferably the coding region thereof, conferred the production of or the increase in sucrose compared with the wild type control.

The nucleic acid sequence of AvinDRAFT_{—}2754 from Azotobacter vinelandii, e.g. as shown in the respective line in column 5 of Table I, application no. 17, has been published in D. And the activity of the gene product thereof is the activity of acyl-CoA synthase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “acyl-CoA synthase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said AvinDRAFT_{—}2754, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}2754, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}2754, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}2754, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “acyl-CoA synthase”, preferably it is the molecule of section (a) or (b) of this paragraph.

In particular, it was observed that in plants, especially in Arabidopsis thaliana, increasing or generating the activity of a gene product non-targeted with the activity of a “acyl-CoA synthase”, preferably being encoded by a gene comprising the nucleic acid sequence SEQ ID NO. 28040, preferably the coding region thereof, conferred the production of or the increase in glucose-1-phosphate compared with the wild type control.

The nucleic acid sequence of AvinDRAFT_{—}3028 from Azotobacter vinelandii, e.g. as shown in the respective line in column 5 of Table I, application no. 17, has been published in D. And the activity of the gene product thereof is the activity of 2-oxoglutarate dehydrogenase E1 subunit.

Accordingly, in one embodiment, the process of the present invention for producing glucose in a non-human organism, like a microorganism or a plant or a part thereof, comprises increasing or generating the activity of a gene product with the activity of a gene product conferring the activity of “2-oxoglutarate dehydrogenase E1 subunit”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said AvinDRAFT_{—}3028, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}3028, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}3028, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}3028, and preferably the activity is increased non-targeted, whereby the respective line disclose in column 7 the fine chemical glucose.

Accordingly, in one embodiment, the molecule which activity is to be increased in the process of the invention is the gene product with an activity as a “2-oxoglutarate dehydrogenase E1 subunit”, preferably it is the molecule of section (a) or (b) of this paragraph.

In particular, it was observed that in plants, especially in Arabidopsis thaliana, increasing or generating the activity of a gene product non-targeted with the activity of a “2-oxoglutarate dehydrogenase E1 subunit”, preferably being encoded by a gene comprising the nucleic acid sequence SEQ ID NO. 6075, preferably the coding region thereof, conferred the production of or the increase in glucose compared with the wild type control.

The nucleic acid sequence of AvinDRAFT_{—}3028 from Azotobacter vinelandii, e.g. as shown in the respective line in column 5 of Table I, application no. 17, has been published in D. And the activity of the gene product thereof is the activity of 2-oxoglutarate dehydrogenase E1 subunit.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “2-oxoglutarate dehydrogenase E1 subunit”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said AvinDRAFT_{—}3028, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}3028, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}3028, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}3028, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “2-oxoglutarate dehydrogenase E1 subunit”, preferably it is the molecule of section (a) or (b) of this paragraph.

In particular, it was observed that in plants, especially in Arabidopsis thaliana, increasing or generating the activity of a gene product non-targeted with the activity of a “2-oxoglutarate dehydrogenase E1 subunit”, preferably being encoded by a gene comprising the nucleic acid sequence SEQ ID NO. 6075, preferably the coding region thereof, conferred the production of or the increase in glucose-1-phosphate compared with the wild type control.

The nucleic acid sequence of AvinDRAFT_{—}3028 from Azotobacter vinelandii, e.g. as shown in the respective line in column 5 of Table I, application no. 17, has been published in D. And the activity of the gene product thereof is the activity of 2-oxoglutarate dehydrogenase E1 subunit.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “2-oxoglutarate dehydrogenase E1 subunit”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said AvinDRAFT_{—}3028, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}3028, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}3028, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}3028, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “2-oxoglutarate dehydrogenase E1 subunit”, preferably it is the molecule of section (a) or (b) of this paragraph.

In particular, it was observed that in plants, especially in Arabidopsis thaliana, increasing or generating the activity of a gene product non-targeted with the activity of a “2-oxoglutarate dehydrogenase E1 subunit”, preferably being encoded by a gene comprising the nucleic acid sequence SEQ ID NO. 6075, preferably the coding region thereof, conferred the production of or the increase in myo-inositol compared with the wild type control.

The nucleic acid sequence of AvinDRAFT_{—}3028 from Azotobacter vinelandii, e.g. as shown in the respective line in column 5 of Table I, application no. 17, has been published in D. And the activity of the gene product thereof is the activity of 2-oxoglutarate dehydrogenase E1 subunit.

Accordingly, in one embodiment, the process of the present invention for producing raffinose in a non-human organism, like a microorganism or a plant or a part thereof, comprises increasing or generating the activity of a gene product with the activity of a gene product conferring the activity of “2-oxoglutarate dehydrogenase E1 subunit”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said AvinDRAFT_{—}3028, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}3028, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}3028, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}3028, and preferably the activity is increased non-targeted, whereby the respective line disclose in column 7 the fine chemical raffinose.

Accordingly, in one embodiment, the molecule which activity is to be increased in the process of the invention is the gene product with an activity as a “2-oxoglutarate dehydrogenase E1 subunit”, preferably it is the molecule of section (a) or (b) of this paragraph.

In particular, it was observed that in plants, especially in Arabidopsis thaliana, increasing or generating the activity of a gene product non-targeted with the activity of a “2-oxoglutarate dehydrogenase E1 subunit”, preferably being encoded by a gene comprising the nucleic acid sequence SEQ ID NO. 6075, preferably the coding region thereof, conferred the production of or the increase in raffinose compared with the wild type control.

The nucleic acid sequence of AvinDRAFT_{—}3028 from Azotobacter vinelandii, e.g. as shown in the respective line in column 5 of Table I, application no. 17, has been published in D. And the activity of the gene product thereof is the activity of 2-oxoglutarate dehydrogenase E1 subunit.

Accordingly, in one embodiment, the process of the present invention for producing sedoheptulose-7-phosphate in a non-human organism, like a microorganism or a plant or a part thereof, comprises increasing or generating the activity of a gene product with the activity of a gene product conferring the activity of “2-oxoglutarate dehydrogenase E1 subunit”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical sedoheptulose-7-phosphate), application no. 17, preferably the coding region thereof, or a homolog or a fragment thereof, and being depicted in the same respective line as said AvinDRAFT_{—}3028, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}3028, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}3028, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}3028, and preferably the activity is increased non-targeted, whereby the respective line disclose in column 7 the fine chemical sedoheptulose-7-phosphate.

Accordingly, in one embodiment, the molecule which activity is to be increased in the process of the invention is the gene product with an activity as a “2-oxoglutarate dehydrogenase E1 subunit”, preferably it is the molecule of section (a) or (b) of this paragraph.

In particular, it was observed that in plants, especially in Arabidopsis thaliana, increasing or generating the activity of a gene product non-targeted with the activity of a “2-oxoglutarate dehydrogenase E1 subunit”, preferably being encoded by a gene comprising the nucleic acid sequence SEQ ID NO. 6075, preferably the coding region thereof, conferred the production of or the increase in sedoheptulose-7-phosphate compared with the wild type control.

The nucleic acid sequence of AvinDRAFT_{—}3135 from Azotobacter vinelandii, e.g. as shown in the respective line in column 5 of Table I, application no. 17, has been published in D. And the activity of the gene product thereof is the activity of GDP-mannose dehydrogenase.

Accordingly, in one embodiment, the process of the present invention for producing raffinose in a non-human organism, like a microorganism or a plant or a part thereof, comprises increasing or generating the activity of a gene product with the activity of a gene product conferring the activity of “GDP-mannose dehydrogenase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said AvinDRAFT_{—}3135, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}3135, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}3135, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}3135, and preferably the activity is increased non-targeted, whereby the respective line disclose in column 7 the fine chemical raffinose.

Accordingly, in one embodiment, the molecule which activity is to be increased in the process of the invention is the gene product with an activity as a “GDP-mannose dehydrogenase”, preferably it is the molecule of section (a) or (b) of this paragraph.

In particular, it was observed that in plants, especially in Arabidopsis thaliana, increasing or generating the activity of a gene product non-targeted with the activity of a “GDP-mannose dehydrogenase”, preferably being encoded by a gene comprising the nucleic acid sequence SEQ ID NO. 72346, preferably the coding region thereof, conferred the production of or the increase in raffinose compared with the wild type control.

The nucleic acid sequence of AvinDRAFT_{—}3209 from Azotobacter vinelandii, e.g. as shown in the respective line in column 5 of Table I, application no. 17, has been published in D. And the activity of the gene product thereof is the activity of transcriptional regulator.

Accordingly, in one embodiment, the process of the present invention for producing fructose in a non-human organism, like a microorganism or a plant or a part thereof, comprises increasing or generating the activity of a gene product with the activity of a gene product conferring the activity of “transcriptional regulator”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said AvinDRAFT_{—}3209, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}3209, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}3209, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}3209, and preferably the activity is increased non-targeted, whereby the respective line disclose in column 7 the fine chemical fructose.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 29286, preferably the coding region thereof, conferred the production of or the increase in fructose compared with the wild type control.

The nucleic acid sequence of AvinDRAFT_{—}3209 from Azotobacter vinelandii, e.g. as shown in the respective line in column 5 of Table I, application no. 17, has been published in D. And the activity of the gene product thereof is the activity of transcriptional regulator.

Accordingly, in one embodiment, the process of the present invention for producing glucose in a non-human organism, like a microorganism or a plant or a part thereof, comprises increasing or generating the activity of a gene product with the activity of a gene product conferring the activity of “transcriptional regulator”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said AvinDRAFT_{—}3209, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}3209, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}3209, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}3209, and preferably the activity is increased non-targeted, whereby the respective line disclose in column 7 the fine chemical glucose.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 29286, preferably the coding region thereof, conferred the production of or the increase in glucose compared with the wild type control.

The nucleic acid sequence of AvinDRAFT_{—}3209 from Azotobacter vinelandii, e.g. as shown in the respective line in column 5 of Table I, application no. 17, has been published in D. And the activity of the gene product thereof is the activity of transcriptional regulator.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “transcriptional regulator”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said AvinDRAFT_{—}3209, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}3209, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}3209, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}3209, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 29286, preferably the coding region thereof, conferred the production of or the increase in glucose-1-phosphate compared with the wild type control.

The nucleic acid sequence of AvinDRAFT_{—}3253 from Azotobacter vinelandii, e.g. as shown in the respective line in column 5 of Table I, application no. 17, has been published in D. And the activity of the gene product thereof is the activity of oxidoreductase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “oxidoreductase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said AvinDRAFT_{—}3253, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}3253, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}3253, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}3253, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 29500, preferably the coding region thereof, conferred the production of or the increase in glucose-1-phosphate compared with the wild type control.

The nucleic acid sequence of AvinDRAFT_{—}3577 from Azotobacter vinelandii, e.g. as shown in the respective line in column 5 of Table I, application no. 17, has been published in D. And the activity of the gene product thereof is the activity of ThiF family protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “ThiF family protein”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said AvinDRAFT_{—}3577, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}3577, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}3577, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}3577, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “ThiF family protein”, preferably it is the molecule of section (a) or (b) of this paragraph.

In particular, it was observed that in plants, especially in Arabidopsis thaliana, increasing or generating the activity of a gene product non-targeted with the activity of a “ThiF family protein”, preferably being encoded by a gene comprising the nucleic acid sequence SEQ ID NO. 111155, preferably the coding region thereof, conferred the production of or the increase in myo-inositol compared with the wild type control.

The nucleic acid sequence of AvinDRAFT_{—}3605 from Azotobacter vinelandii, e.g. as shown in the respective line in column 5 of Table I, application no. 17, has been published in D. And the activity of the gene product thereof is the activity of CTP synthase.

Accordingly, in one embodiment, the process of the present invention for producing fructose in a non-human organism, like a microorganism or a plant or a part thereof, comprises increasing or generating the activity of a gene product with the activity of a gene product conferring the activity of “CTP synthase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said AvinDRAFT_{—}3605, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}3605, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}3605, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}3605, and preferably the activity is increased non-targeted, whereby the respective line disclose in column 7 the fine chemical fructose.

Accordingly, in one embodiment, the molecule which activity is to be increased in the process of the invention is the gene product with an activity as a “CTP synthase”, preferably it is the molecule of section (a) or (b) of this paragraph.

In particular, it was observed that in plants, especially in Arabidopsis thaliana, increasing or generating the activity of a gene product non-targeted with the activity of a “CTP synthase”, preferably being encoded by a gene comprising the nucleic acid sequence SEQ ID NO. 31026, preferably the coding region thereof, conferred the production of or the increase in fructose compared with the wild type control.

The nucleic acid sequence of AvinDRAFT_{—}3605 from Azotobacter vinelandii, e.g. as shown in the respective line in column 5 of Table I, application no. 17, has been published in D. And the activity of the gene product thereof is the activity of CTP synthase.

Accordingly, in one embodiment, the process of the present invention for producing glucose in a non-human organism, like a microorganism or a plant or a part thereof, comprises increasing or generating the activity of a gene product with the activity of a gene product conferring the activity of “CTP synthase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said AvinDRAFT_{—}3605, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}3605, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}3605, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}3605, and preferably the activity is increased non-targeted, whereby the respective line disclose in column 7 the fine chemical glucose.

Accordingly, in one embodiment, the molecule which activity is to be increased in the process of the invention is the gene product with an activity as a “CTP synthase”, preferably it is the molecule of section (a) or (b) of this paragraph.

In particular, it was observed that in plants, especially in Arabidopsis thaliana, increasing or generating the activity of a gene product non-targeted with the activity of a “CTP synthase”, preferably being encoded by a gene comprising the nucleic acid sequence SEQ ID NO. 31026, preferably the coding region thereof, conferred the production of or the increase in glucose compared with the wild type control.

The nucleic acid sequence of AvinDRAFT_{—}3605 from Azotobacter vinelandii, e.g. as shown in the respective line in column 5 of Table I, application no. 17, has been published in D. And the activity of the gene product thereof is the activity of CTP synthase.

Accordingly, in one embodiment, the process of the present invention for producing sucrose in a non-human organism, like a microorganism or a plant or a part thereof, comprises increasing or generating the activity of a gene product with the activity of a gene product conferring the activity of “CTP synthase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said AvinDRAFT_{—}3605, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}3605, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}3605, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}3605, and preferably the activity is increased non-targeted, whereby the respective line disclose in column 7 the fine chemical sucrose.

Accordingly, in one embodiment, the molecule which activity is to be increased in the process of the invention is the gene product with an activity as a “CTP synthase”, preferably it is the molecule of section (a) or (b) of this paragraph.

In particular, it was observed that in plants, especially in Arabidopsis thaliana, increasing or generating the activity of a gene product non-targeted with the activity of a “CTP synthase”, preferably being encoded by a gene comprising the nucleic acid sequence SEQ ID NO. 31026, preferably the coding region thereof, conferred the production of or the increase in sucrose compared with the wild type control.

The nucleic acid sequence of AvinDRAFT_{—}3616 from Azotobacter vinelandii, e.g. as shown in the respective line in column 5 of Table I, application no. 17, has been published in D. And the activity of the gene product thereof is the activity of protein-L-isoaspartate O-methyltransferase.

Accordingly, in one embodiment, the process of the present invention for producing raffinose in a non-human organism, like a microorganism or a plant or a part thereof, comprises increasing or generating the activity of a gene product with the activity of a gene product conferring the activity of “protein-L-isoaspartate O-methyltransferase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said AvinDRAFT_{—}3616, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}3616, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}3616, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}3616, and preferably the activity is increased non-targeted, whereby the respective line disclose in column 7 the fine chemical raffinose.

Accordingly, in one embodiment, the molecule which activity is to be increased in the process of the invention is the gene product with an activity as a “protein-L-isoaspartate O-methyltransferase”, preferably it is the molecule of section (a) or (b) of this paragraph.

In particular, it was observed that in plants, especially in Arabidopsis thaliana, increasing or generating the activity of a gene product non-targeted with the activity of a “protein-L-isoaspartate O-methyltransferase”, preferably being encoded by a gene comprising the nucleic acid sequence SEQ ID NO. 143121, preferably the coding region thereof, conferred the production of or the increase in raffinose compared with the wild type control.

The nucleic acid sequence of AvinDRAFT_{—}3629 from Azotobacter vinelandii, e.g. as shown in the respective line in column 5 of Table I, application no. 17, has been published in D. And the activity of the gene product thereof is the activity of recombinase A.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “recombinase A”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said AvinDRAFT_{—}3629, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}3629, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}3629, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}3629, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “recombinase A”, preferably it is the molecule of section (a) or (b) of this paragraph.

In particular, it was observed that in plants, especially in Arabidopsis thaliana, increasing or generating the activity of a gene product non-targeted with the activity of a “recombinase A”, preferably being encoded by a gene comprising the nucleic acid sequence SEQ ID NO. 114231, preferably the coding region thereof, conferred the production of or the increase in myo-inositol compared with the wild type control.

The nucleic acid sequence of AvinDRAFT_{—}3629 from Azotobacter vinelandii, e.g. as shown in the respective line in column 5 of Table I, application no. 17, has been published in D. And the activity of the gene product thereof is the activity of recombinase A.

Accordingly, in one embodiment, the process of the present invention for producing raffinose in a non-human organism, like a microorganism or a plant or a part thereof, comprises increasing or generating the activity of a gene product with the activity of a gene product conferring the activity of “recombinase A”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said AvinDRAFT_{—}3629, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}3629, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}3629, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}3629, and preferably the activity is increased non-targeted, whereby the respective line disclose in column 7 the fine chemical raffinose.

Accordingly, in one embodiment, the molecule which activity is to be increased in the process of the invention is the gene product with an activity as a “recombinase A”, preferably it is the molecule of section (a) or (b) of this paragraph.

In particular, it was observed that in plants, especially in Arabidopsis thaliana, increasing or generating the activity of a gene product non-targeted with the activity of a “recombinase A”, preferably being encoded by a gene comprising the nucleic acid sequence SEQ ID NO. 114231, preferably the coding region thereof, conferred the production of or the increase in raffinose compared with the wild type control.

The nucleic acid sequence of AvinDRAFT_{—}4265 from Azotobacter vinelandii, e.g. as shown in the respective line in column 5 of Table I, application no. 17, has been published in D. And the activity of the gene product thereof is the activity of acetyltransferase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “acetyltransferase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said AvinDRAFT_{—}4265, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}4265, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}4265, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}4265, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 143398, preferably the coding region thereof, conferred the production of or the increase in myo-inositol compared with the wild type control.

The nucleic acid sequence of AvinDRAFT_{—}4265 from Azotobacter vinelandii, e.g. as shown in the respective line in column 5 of Table I, application no. 17, has been published in D. And the activity of the gene product thereof is the activity of acetyltransferase.

Accordingly, in one embodiment, the process of the present invention for producing raffinose in a non-human organism, like a microorganism or a plant or a part thereof, comprises increasing or generating the activity of a gene product with the activity of a gene product conferring the activity of “acetyltransferase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said AvinDRAFT_{—}4265, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}4265, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}4265, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}4265, and preferably the activity is increased non-targeted, whereby the respective line disclose in column 7 the fine chemical raffinose.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 143398, preferably the coding region thereof, conferred the production of or the increase in raffinose compared with the wild type control.

The nucleic acid sequence of AvinDRAFT_{—}4420 from Azotobacter vinelandii, e.g. as shown in the respective line in column 5 of Table I, application no. 17, has been published in D. And the activity of the gene product thereof is the activity of enoyl-CoA hydratase.

Accordingly, in one embodiment, the process of the present invention for producing raffinose in a non-human organism, like a microorganism or a plant or a part thereof, comprises increasing or generating the activity of a gene product with the activity of a gene product conferring the activity of “enoyl-CoA hydratase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said AvinDRAFT_{—}4420, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}4420, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}4420, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}4420, and preferably the activity is increased non-targeted, whereby the respective line disclose in column 7 the fine chemical raffinose.

Accordingly, in one embodiment, the molecule which activity is to be increased in the process of the invention is the gene product with an activity as a “enoyl-CoA hydratase”, preferably it is the molecule of section (a) or (b) of this paragraph.

In particular, it was observed that in plants, especially in Arabidopsis thaliana, increasing or generating the activity of a gene product non-targeted with the activity of a “enoyl-CoA hydratase”, preferably being encoded by a gene comprising the nucleic acid sequence SEQ ID NO. 73038, preferably the coding region thereof, conferred the production of or the increase in raffinose compared with the wild type control.

The nucleic acid sequence of AvinDRAFT_{—}4606 from Azotobacter vinelandii, e.g. as shown in the respective line in column 5 of Table I, application no. 17, has been published in D. And the activity of the gene product thereof is the activity of NADH-quinone oxidoreductase subunit.

Accordingly, in one embodiment, the process of the present invention for producing fructose in a non-human organism, like a microorganism or a plant or a part thereof, comprises increasing or generating the activity of a gene product with the activity of a gene product conferring the activity of “NADH-quinone oxidoreductase subunit”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said AvinDRAFT_{—}4606, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}4606, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}4606, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}4606, and preferably the activity is increased non-targeted, whereby the respective line disclose in column 7 the fine chemical fructose.

Accordingly, in one embodiment, the molecule which activity is to be increased in the process of the invention is the gene product with an activity as a “NADH-quinone oxidoreductase subunit”, preferably it is the molecule of section (a) or (b) of this paragraph.

In particular, it was observed that in plants, especially in Arabidopsis thaliana, increasing or generating the activity of a gene product non-targeted with the activity of a “NADH-quinone oxidoreductase subunit”, preferably being encoded by a gene comprising the nucleic acid sequence SEQ ID NO. 73719, preferably the coding region thereof, conferred the production of or the increase in fructose compared with the wild type control.

The nucleic acid sequence of AvinDRAFT_{—}4606 from Azotobacter vinelandii, e.g. as shown in the respective line in column 5 of Table I, application no. 17, has been published in D. And the activity of the gene product thereof is the activity of NADH-quinone oxidoreductase subunit.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “NADH-quinone oxidoreductase subunit”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said AvinDRAFT_{—}4606, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}4606, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}4606, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}4606, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “NADH-quinone oxidoreductase subunit”, preferably it is the molecule of section (a) or (b) of this paragraph.

In particular, it was observed that in plants, especially in Arabidopsis thaliana, increasing or generating the activity of a gene product non-targeted with the activity of a “NADH-quinone oxidoreductase subunit”, preferably being encoded by a gene comprising the nucleic acid sequence SEQ ID NO. 73719, preferably the coding region thereof, conferred the production of or the increase in glucose-1-phosphate compared with the wild type control.

The nucleic acid sequence of AvinDRAFT_{—}4606 from Azotobacter vinelandii, e.g. as shown in the respective line in column 5 of Table I, application no. 17, has been published in D. And the activity of the gene product thereof is the activity of NADH-quinone oxidoreductase subunit.

Accordingly, in one embodiment, the process of the present invention for producing sucrose in a non-human organism, like a microorganism or a plant or a part thereof, comprises increasing or generating the activity of a gene product with the activity of a gene product conferring the activity of “NADH-quinone oxidoreductase subunit”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said AvinDRAFT_{—}4606, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}4606, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}4606, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}4606, and preferably the activity is increased non-targeted, whereby the respective line disclose in column 7 the fine chemical sucrose.

Accordingly, in one embodiment, the molecule which activity is to be increased in the process of the invention is the gene product with an activity as a “NADH-quinone oxidoreductase subunit”, preferably it is the molecule of section (a) or (b) of this paragraph.

In particular, it was observed that in plants, especially in Arabidopsis thaliana, increasing or generating the activity of a gene product non-targeted with the activity of a “NADH-quinone oxidoreductase subunit”, preferably being encoded by a gene comprising the nucleic acid sequence SEQ ID NO. 73719, preferably the coding region thereof, conferred the production of or the increase in sucrose compared with the wild type control.

The nucleic acid sequence of AvinDRAFT_{—}4847 from Azotobacter vinelandii, e.g. as shown in the respective line in column 5 of Table I, application no. 17, has been published in D. And the activity of the gene product thereof is the activity of isopropylmalate isomerase large subunit.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “isopropylmalate isomerase large subunit”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said AvinDRAFT_{—}4847, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}4847, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}4847, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}4847, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “isopropylmalate isomerase large subunit”, preferably it is the molecule of section (a) or (b) of this paragraph.

In particular, it was observed that in plants, especially in Arabidopsis thaliana, increasing or generating the activity of a gene product non-targeted with the activity of a “isopropylmalate isomerase large subunit”, preferably being encoded by a gene comprising the nucleic acid sequence SEQ ID NO. 102941, preferably the coding region thereof, conferred the production of or the increase in 3,4,5-trihydroxypentanoic acid compared with the wild type control.

The nucleic acid sequence of AvinDRAFT_{—}5103 from Azotobacter vinelandii, e.g. as shown in the respective line in column 5 of Table I, application no. 17, has been published in D. And the activity of the gene product thereof is the activity of hydrolase.

Accordingly, in one embodiment, the process of the present invention for producing fructose in a non-human organism, like a microorganism or a plant or a part thereof, comprises increasing or generating the activity of a gene product with the activity of a gene product conferring the activity of “hydrolase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said AvinDRAFT_{—}5103, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}5103, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}5103, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}5103, and preferably the activity is increased non-targeted, whereby the respective line disclose in column 7 the fine chemical fructose.

Accordingly, in one embodiment, the molecule which activity is to be increased in the process of the invention is the gene product with an activity as a “hydrolase”, preferably it is the molecule of section (a) or (b) of this paragraph.

In particular, it was observed that in plants, especially in Arabidopsis thaliana, increasing or generating the activity of a gene product non-targeted with the activity of a “hydrolase”, preferably being encoded by a gene comprising the nucleic acid sequence SEQ ID NO. 6510, preferably the coding region thereof, conferred the production of or the increase in fructose compared with the wild type control.

The nucleic acid sequence of AvinDRAFT_{—}5103 from Azotobacter vinelandii, e.g. as shown in the respective line in column 5 of Table I, application no. 17, has been published in D. And the activity of the gene product thereof is the activity of hydrolase.

Accordingly, in one embodiment, the process of the present invention for producing glucose in a non-human organism, like a microorganism or a plant or a part thereof, comprises increasing or generating the activity of a gene product with the activity of a gene product conferring the activity of “hydrolase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said AvinDRAFT_{—}5103, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}5103, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}5103, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}5103, and preferably the activity is increased non-targeted, whereby the respective line disclose in column 7 the fine chemical glucose.

Accordingly, in one embodiment, the molecule which activity is to be increased in the process of the invention is the gene product with an activity as a “hydrolase”, preferably it is the molecule of section (a) or (b) of this paragraph.

In particular, it was observed that in plants, especially in Arabidopsis thaliana, increasing or generating the activity of a gene product non-targeted with the activity of a “hydrolase”, preferably being encoded by a gene comprising the nucleic acid sequence SEQ ID NO. 6510, preferably the coding region thereof, conferred the production of or the increase in glucose compared with the wild type control.

The nucleic acid sequence of AvinDRAFT_{—}5103 from Azotobacter vinelandii, e.g. as shown in the respective line in column 5 of Table I, application no. 17, has been published in D. And the activity of the gene product thereof is the activity of hydrolase.

Accordingly, in one embodiment, the process of the present invention for producing sucrose in a non-human organism, like a microorganism or a plant or a part thereof, comprises increasing or generating the activity of a gene product with the activity of a gene product conferring the activity of “hydrolase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said AvinDRAFT_{—}5103, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}5103, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}5103, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}5103, and preferably the activity is increased non-targeted, whereby the respective line disclose in column 7 the fine chemical sucrose.

Accordingly, in one embodiment, the molecule which activity is to be increased in the process of the invention is the gene product with an activity as a “hydrolase”, preferably it is the molecule of section (a) or (b) of this paragraph.

In particular, it was observed that in plants, especially in Arabidopsis thaliana, increasing or generating the activity of a gene product non-targeted with the activity of a “hydrolase”, preferably being encoded by a gene comprising the nucleic acid sequence SEQ ID NO. 6510, preferably the coding region thereof, conferred the production of or the increase in sucrose compared with the wild type control.

The nucleic acid sequence of AvinDRAFT_{—}5246 from Azotobacter vinelandii, e.g. as shown in the respective line in column 5 of Table I, application no. 17, has been published in D. And the activity of the gene product thereof is the activity of glutamate-ammonia-ligase.

Accordingly, in one embodiment, the process of the present invention for producing fructose in a non-human organism, like a microorganism or a plant or a part thereof, comprises increasing or generating the activity of a gene product with the activity of a gene product conferring the activity of “glutamate-ammonia-ligase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said AvinDRAFT_{—}5246, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}5246, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}5246, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}5246, and preferably the activity is increased non-targeted, whereby the respective line disclose in column 7 the fine chemical fructose.

Accordingly, in one embodiment, the molecule which activity is to be increased in the process of the invention is the gene product with an activity as a “glutamate-ammonia-ligase”, preferably it is the molecule of section (a) or (b) of this paragraph.

In particular, it was observed that in plants, especially in Arabidopsis thaliana, increasing or generating the activity of a gene product non-targeted with the activity of a “glutamate-ammonia-ligase”, preferably being encoded by a gene comprising the nucleic acid sequence SEQ ID NO. 32037, preferably the coding region thereof, conferred the production of or the increase in fructose compared with the wild type control.

The nucleic acid sequence of AvinDRAFT_{—}5246 from Azotobacter vinelandii, e.g. as shown in the respective line in column 5 of Table I, application no. 17, has been published in D. And the activity of the gene product thereof is the activity of glutamate-ammonia-ligase.

Accordingly, in one embodiment, the process of the present invention for producing glucose in a non-human organism, like a microorganism or a plant or a part thereof, comprises increasing or generating the activity of a gene product with the activity of a gene product conferring the activity of “glutamate-ammonia-ligase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said AvinDRAFT_{—}5246, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}5246, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}5246, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}5246, and preferably the activity is increased non-targeted, whereby the respective line disclose in column 7 the fine chemical glucose.

Accordingly, in one embodiment, the molecule which activity is to be increased in the process of the invention is the gene product with an activity as a “glutamate-ammonia-ligase”, preferably it is the molecule of section (a) or (b) of this paragraph.

In particular, it was observed that in plants, especially in Arabidopsis thaliana, increasing or generating the activity of a gene product non-targeted with the activity of a “glutamate-ammonia-ligase”, preferably being encoded by a gene comprising the nucleic acid sequence SEQ ID NO. 32037, preferably the coding region thereof, conferred the production of or the increase in glucose compared with the wild type control.

The nucleic acid sequence of AvinDRAFT_{—}5246 from Azotobacter vinelandii, e.g. as shown in the respective line in column 5 of Table I, application no. 17, has been published in D. And the activity of the gene product thereof is the activity of glutamate-ammonia-ligase.

Accordingly, in one embodiment, the process of the present invention for producing sucrose in a non-human organism, like a microorganism or a plant or a part thereof, comprises increasing or generating the activity of a gene product with the activity of a gene product conferring the activity of “glutamate-ammonia-ligase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said AvinDRAFT_{—}5246, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}5246, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}5246, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}5246, and preferably the activity is increased non-targeted, whereby the respective line disclose in column 7 the fine chemical sucrose.

Accordingly, in one embodiment, the molecule which activity is to be increased in the process of the invention is the gene product with an activity as a “glutamate-ammonia-ligase”, preferably it is the molecule of section (a) or (b) of this paragraph.

In particular, it was observed that in plants, especially in Arabidopsis thaliana, increasing or generating the activity of a gene product non-targeted with the activity of a “glutamate-ammonia-ligase”, preferably being encoded by a gene comprising the nucleic acid sequence SEQ ID NO. 32037, preferably the coding region thereof, conferred the production of or the increase in sucrose compared with the wild type control.

The nucleic acid sequence of AvinDRAFT_{—}5292 from Azotobacter vinelandii, e.g. as shown in the respective line in column 5 of Table I, application no. 17, has been published in D. And the activity of the gene product thereof is the activity of fumarylacetoacetate hydrolase.

Accordingly, in one embodiment, the process of the present invention for producing fructose in a non-human organism, like a microorganism or a plant or a part thereof, comprises increasing or generating the activity of a gene product with the activity of a gene product conferring the activity of “fumarylacetoacetate hydrolase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said AvinDRAFT_{—}5292, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}5292, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}5292, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}5292, and preferably the activity is increased non-targeted, whereby the respective line disclose in column 7 the fine chemical fructose.

Accordingly, in one embodiment, the molecule which activity is to be increased in the process of the invention is the gene product with an activity as a “fumarylacetoacetate hydrolase”, preferably it is the molecule of section (a) or (b) of this paragraph.

In particular, it was observed that in plants, especially in Arabidopsis thaliana, increasing or generating the activity of a gene product non-targeted with the activity of a “fumarylacetoacetate hydrolase”, preferably being encoded by a gene comprising the nucleic acid sequence SEQ ID NO. 32308, preferably the coding region thereof, conferred the production of or the increase in fructose compared with the wild type control.

The nucleic acid sequence of AvinDRAFT_{—}5967 from Azotobacter vinelandii, e.g. as shown in the respective line in column 5 of Table I, application no. 17, has been published in D. And the activity of the gene product thereof is the activity of glycosyltransferase.

Accordingly, in one embodiment, the process of the present invention for producing raffinose in a non-human organism, like a microorganism or a plant or a part thereof, comprises increasing or generating the activity of a gene product with the activity of a gene product conferring the activity of “glycosyltransferase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said AvinDRAFT_{—}5967, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}5967, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}5967, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}5967, and preferably the activity is increased non-targeted, whereby the respective line disclose in column 7 the fine chemical raffinose.

Accordingly, in one embodiment, the molecule which activity is to be increased in the process of the invention is the gene product with an activity as a “glycosyltransferase”, preferably it is the molecule of section (a) or (b) of this paragraph.

In particular, it was observed that in plants, especially in Arabidopsis thaliana, increasing or generating the activity of a gene product non-targeted with the activity of a “glycosyltransferase”, preferably being encoded by a gene comprising the nucleic acid sequence SEQ ID NO. 143495, preferably the coding region thereof, conferred the production of or the increase in raffinose compared with the wild type control.

The nucleic acid sequence of AvinDRAFT_{—}6075 from Azotobacter vinelandii, e.g. as shown in the respective line in column 5 of Table I, application no. 17, has been published in D. And the activity of the gene product thereof is the activity of peptidyl-prolyl cis-trans isomerase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “peptidyl-prolyl cis-trans isomerase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said AvinDRAFT_{—}6075, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}6075, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}6075, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}6075, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “peptidyl-prolyl cis-trans isomerase”, preferably it is the molecule of section (a) or (b) of this paragraph.

In particular, it was observed that in plants, especially in Arabidopsis thaliana, increasing or generating the activity of a gene product non-targeted with the activity of a “peptidyl-prolyl cistrans isomerase”, preferably being encoded by a gene comprising the nucleic acid sequence SEQ ID NO. 6674, preferably the coding region thereof, conferred the production of or the increase in myo-inositol compared with the wild type control.

The nucleic acid sequence of AVINDRAFT_{—}6261 from Azotobacter vinelandii, e.g. as shown in the respective line in column 5 of Table I, application no. 17, has been published in D. And the activity of the gene product thereof is the activity of NAD(P)H dehydrogenase (quinone).

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “NAD(P)H dehydrogenase (quinone)”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said AVINDRAFT_{—}6261, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AVINDRAFT_{—}6261, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AVINDRAFT_{—}6261, or a functional equivalent or a homolog thereof as depicted in column 8 of Table II, application no. 17, preferably a homolog or functional equivalent as depicted in column 8 of Table II B, application no. 17, and being depicted in the same respective line as said AVINDRAFT_{—}6261, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “NAD(P)H dehydrogenase (quinone)”, preferably it is the molecule of section (a) or (b) of this paragraph.

In particular, it was observed that in plants, especially in Arabidopsis thaliana, increasing or generating the activity of a gene product non-targeted with the activity of a “NAD(P)H dehydrogenase (quinone)”, preferably being encoded by a gene comprising the nucleic acid sequence SEQ ID NO. 143614, preferably the coding region thereof, conferred the production of or the increase in 3,4,5-trihydroxypentanoic acid compared with the wild type control.

The nucleic acid sequence of AvinDRAFT_{—}6679 from Azotobacter vinelandii, e.g. as shown in the respective line in column 5 of Table I, application no. 17, has been published in D. And the activity of the gene product thereof is the activity of electron transfer flavoprotein subunit beta.

Accordingly, in one embodiment, the process of the present invention for producing fructose in a non-human organism, like a microorganism or a plant or a part thereof, comprises increasing or generating the activity of a gene product with the activity of a gene product conferring the activity of “electron transfer flavoprotein subunit beta”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said AvinDRAFT_{—}6679, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}6679, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}6679, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}6679, and preferably the activity is increased non-targeted, whereby the respective line disclose in column 7 the fine chemical fructose.

Accordingly, in one embodiment, the molecule which activity is to be increased in the process of the invention is the gene product with an activity as a “electron transfer flavoprotein subunit beta”, preferably it is the molecule of section (a) or (b) of this paragraph.

In particular, it was observed that in plants, especially in Arabidopsis thaliana, increasing or generating the activity of a gene product non-targeted with the activity of a “electron transfer flavoprotein subunit beta”, preferably being encoded by a gene comprising the nucleic acid sequence SEQ ID NO. 74729, preferably the coding region thereof, conferred the production of or the increase in fructose compared with the wild type control.

The nucleic acid sequence of AvinDRAFT_{—}6679 from Azotobacter vinelandii, e.g. as shown in the respective line in column 5 of Table I, application no. 17, has been published in D. And the activity of the gene product thereof is the activity of electron transfer flavoprotein subunit beta.

Accordingly, in one embodiment, the process of the present invention for producing glucose in a non-human organism, like a microorganism or a plant or a part thereof, comprises increasing or generating the activity of a gene product with the activity of a gene product conferring the activity of “electron transfer flavoprotein subunit beta”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said AvinDRAFT_{—}6679, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}6679, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}6679, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}6679, and preferably the activity is increased non-targeted, whereby the respective line disclose in column 7 the fine chemical glucose.

Accordingly, in one embodiment, the molecule which activity is to be increased in the process of the invention is the gene product with an activity as a “electron transfer flavoprotein subunit beta”, preferably it is the molecule of section (a) or (b) of this paragraph.

In particular, it was observed that in plants, especially in Arabidopsis thaliana, increasing or generating the activity of a gene product non-targeted with the activity of a “electron transfer flavoprotein subunit beta”, preferably being encoded by a gene comprising the nucleic acid sequence SEQ ID NO. 74729, preferably the coding region thereof, conferred the production of or the increase in glucose compared with the wild type control.

The nucleic acid sequence of AvinDRAFT_{—}6700 from Azotobacter vinelandii, e.g. as shown in the respective line in column 5 of Table I, application no. 17, has been published in D. And the activity of the gene product thereof is the activity of purine nucleoside phosphorylase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “purine nucleoside phosphorylase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said AvinDRAFT_{—}6700, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}6700, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}6700, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}6700, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “purine nucleoside phosphorylase”, preferably it is the molecule of section (a) or (b) of this paragraph.

In particular, it was observed that in plants, especially in Arabidopsis thaliana, increasing or generating the activity of a gene product non-targeted with the activity of a “purine nucleoside phosphorylase”, preferably being encoded by a gene comprising the nucleic acid sequence SEQ ID NO. 34044, preferably the coding region thereof, conferred the production of or the increase in glucose-1-phosphate compared with the wild type control.

The nucleic acid sequence of AvinDRAFT_{—}6700 from Azotobacter vinelandii, e.g. as shown in the respective line in column 5 of Table I, application no. 17, has been published in D. And the activity of the gene product thereof is the activity of purine nucleoside phosphorylase.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “purine nucleoside phosphorylase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said AvinDRAFT_{—}6700, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}6700, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}6700, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}6700, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “purine nucleoside phosphorylase”, preferably it is the molecule of section (a) or (b) of this paragraph.

In particular, it was observed that in plants, especially in Arabidopsis thaliana, increasing or generating the activity of a gene product non-targeted with the activity of a “purine nucleoside phosphorylase”, preferably being encoded by a gene comprising the nucleic acid sequence SEQ ID NO. 34044, preferably the coding region thereof, conferred the production of or the increase in 3,4,5-trihydroxypentanoic acid compared with the wild type control.

The nucleic acid sequence of AvinDRAFT_{—}6864 from Azotobacter vinelandii, e.g. as shown in the respective line in column 5 of Table I, application no. 17, has been published in D. And the activity of the gene product thereof is the activity of transcriptional regulator protein.

Accordingly, in one embodiment, the process of the present invention for producing raffinose in a non-human organism, like a microorganism or a plant or a part thereof, comprises increasing or generating the activity of a gene product with the activity of a gene product conferring the activity of “transcriptional regulator protein”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said AvinDRAFT_{—}6864, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AvinDRAFT_{—}6864, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AvinDRAFT_{—}6864, or a functional equivalent or a homolog thereof as depicted in column 8 of Table II, application no. 17, preferably a homolog or functional equivalent as depicted in column 8 of Table II B, application no. 17, and being depicted in the same respective line as said AvinDRAFT_{—}6864, and preferably the activity is increased non-targeted, whereby the respective line disclose in column 7 the fine chemical raffinose.

Accordingly, in one embodiment, the molecule which activity is to be increased in the process of the invention is the gene product with an activity as a “transcriptional regulator protein”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator protein”, preferably being encoded by a gene comprising the nucleic acid sequence SEQ ID NO. 34204, preferably the coding region thereof, conferred the production of or the increase in raffinose compared with the wild type control.

The nucleic acid sequence of AX653549 from Oryza sativa, e.g. as shown in the respective line in column 5 of Table I, application no. 17, has been published in G. And the activity of the gene product thereof is the activity of AX653549-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “AX653549-protein”, especially from Oryza sativa or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said AX653549, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AX653549, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AX653549, or a functional equivalent or a homolog thereof as depicted in column 8 of Table II, application no. 17, preferably a homolog or functional equivalent as depicted in column 8 of Table II B, application no. 17, and being depicted in the same respective line as said AX653549, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “AX653549-protein”, preferably it is the molecule of section (a) or (b) of this paragraph.

In particular, it was observed that in plants, especially in Arabidopsis thaliana, increasing or generating the activity of a gene product non-targeted with the activity of a “AX653549-protein”, preferably being encoded by a gene comprising the nucleic acid sequence SEQ ID NO. 34301, preferably the coding region thereof, conferred the production of or the increase in glucose-1-phosphate compared with the wild type control.

The nucleic acid sequence of AY087308 from Arabidopsis thaliana, e.g. as shown in the respective line in column 5 of Table I, application no. 17, has been published in F. And the activity of the gene product thereof is the activity of AY087308-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “AY087308-protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said AY087308, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AY087308, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AY087308, or a functional equivalent or a homolog thereof as depicted in column 8 of Table II, application no. 17, preferably a homolog or functional equivalent as depicted in column 8 of Table II B, application no. 17, and being depicted in the same respective line as said AY087308, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “AY087308-protein”, preferably it is the molecule of section (a) or (b) of this paragraph.

In particular, it was observed that in plants, especially in Arabidopsis thaliana, increasing or generating the activity of a gene product non-targeted with the activity of a “AY087308-protein”, preferably being encoded by a gene comprising the nucleic acid sequence SEQ ID NO. 34602, preferably the coding region thereof, conferred the production of or the increase in glucose-1-phosphate compared with the wild type control.

The nucleic acid sequence of AY087308 from Arabidopsis thaliana, e.g. as shown in the respective line in column 5 of Table I, application no. 17, has been published in F. And the activity of the gene product thereof is the activity of AY087308-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “AY087308-protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said AY087308, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AY087308, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AY087308, or a functional equivalent or a homolog thereof as depicted in column 8 of Table II, application no. 17, preferably a homolog or functional equivalent as depicted in column 8 of Table II B, application no. 17, and being depicted in the same respective line as said AY087308, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “AY087308-protein”, preferably it is the molecule of section (a) or (b) of this paragraph.

In particular, it was observed that in plants, especially in Arabidopsis thaliana, increasing or generating the activity of a gene product non-targeted with the activity of a “AY087308-protein”, preferably being encoded by a gene comprising the nucleic acid sequence SEQ ID NO. 34602, preferably the coding region thereof, conferred the production of or the increase in myo-inositol compared with the wild type control.

The nucleic acid sequence of AY623894 from Zea mays, e.g. as shown in the respective line in column 5 of Table I, application no. 17, is unpublished. And the activity of the gene product thereof is the activity of pyruvate kinase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “pyruvate kinase”, especially from Zea mays or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said AY623894, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AY623894, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AY623894, or a functional equivalent or a homolog thereof as depicted in column 8 of Table II, application no. 17, preferably a homolog or functional equivalent as depicted in column 8 of Table II B, application no. 17, and being depicted in the same respective line as said AY623894, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product non-targeted with the activity of a “pyruvate kinase”, preferably being encoded by a gene comprising the nucleic acid sequence SEQ ID NO. 103433, preferably the coding region thereof, conferred the production of or the increase in glucose-1-phosphate compared with the wild type control.

The nucleic acid sequence of AY623894 from Zea mays, e.g. as shown in the respective line in column 5 of Table I, application no. 17, is unpublished. And the activity of the gene product thereof is the activity of pyruvate kinase.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “pyruvate kinase”, especially from Zea mays or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said AY623894, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said AY623894, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said AY623894, or a functional equivalent or a homolog thereof as depicted in column 8 of Table II, application no. 17, preferably a homolog or functional equivalent as depicted in column 8 of Table II B, application no. 17, and being depicted in the same respective line as said AY623894, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product non-targeted with the activity of a “pyruvate kinase”, preferably being encoded by a gene comprising the nucleic acid sequence SEQ ID NO. 103433, preferably the coding region thereof, conferred the production of or the increase in 3,4,5-trihydroxypentanoic acid compared with the wild type control.

The nucleic acid sequence of B0004 from Escherichia coli, e.g. as shown in the respective line in column 5 of Table I, application no. 17, has been published in B. And the activity of the gene product thereof is the activity of threonine synthase.

Accordingly, in one embodiment, the process of the present invention for producing glucose in a non-human organism, like a microorganism or a plant or a part thereof, comprises increasing or generating the activity of a gene product with the activity of a gene product conferring the activity of “threonine synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B0004, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0004, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0004, or a functional equivalent or a homolog thereof as depicted in column 8 of Table II, application no. 17, preferably a homolog or functional equivalent as depicted in column 8 of Table II B, application no. 17, and being depicted in the same respective line as said B0004, and preferably the activity is increased plastidic, whereby the respective line disclose in column 7 the fine chemical glucose.

Accordingly, in one embodiment, the molecule which activity is to be increased in the process of the invention is the gene product with an activity as a “threonine synthase”, preferably it is the molecule of section (a) or (b) of this paragraph.

In particular, it was observed that in plants, especially in Arabidopsis thaliana, increasing or generating the activity of a gene product plastidic with the activity of a “threonine synthase”, preferably being encoded by a gene comprising the nucleic acid sequence SEQ ID NO. 34889, preferably the coding region thereof, conferred the production of or the increase in glucose compared with the wild type control.

The nucleic acid sequence of B0004 from Escherichia coli, e.g. as shown in the respective line in column 5 of Table I, application no. 17, has been published in B. And the activity of the gene product thereof is the activity of threonine synthase.

Accordingly, in one embodiment, the process of the present invention for producing sucrose in a non-human organism, like a microorganism or a plant or a part thereof, comprises increasing or generating the activity of a gene product with the activity of a gene product conferring the activity of “threonine synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said B0004, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0004, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0004, or a functional equivalent or a homolog thereof as depicted in column 8 of Table II, application no. 17, preferably a homolog or functional equivalent as depicted in column 8 of Table II B, application no. 17, and being depicted in the same respective line as said B0004, and preferably the activity is increased plastidic, whereby the respective line disclose in column 7 the fine chemical sucrose.

Accordingly, in one embodiment, the molecule which activity is to be increased in the process of the invention is the gene product with an activity as a “threonine synthase”, preferably it is the molecule of section (a) or (b) of this paragraph.

In particular, it was observed that in plants, especially in Arabidopsis thaliana, increasing or generating the activity of a gene product plastidic with the activity of a “threonine synthase”, preferably being encoded by a gene comprising the nucleic acid sequence SEQ ID NO. 34889, preferably the coding region thereof, conferred the production of or the increase in sucrose compared with the wild type control.

The nucleic acid sequence of B0050 from Escherichia coli, e.g. as shown in the respective line in column 5 of Table I, application no. 17, has been published in B. And the activity of the gene product thereof is the activity of b0050-protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “b0050-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B0050, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0050, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0050, or a functional equivalent or a homolog thereof as depicted in column 8 of Table II, application no. 17, preferably a homolog or functional equivalent as depicted in column 8 of Table II B, application no. 17, and being depicted in the same respective line as said B0050, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “b0050-protein”, preferably it is the molecule of section (a) or (b) of this paragraph.

In particular, it was observed that in plants, especially in Arabidopsis thaliana, increasing or generating the activity of a gene product non-targeted with the activity of a “b0050-protein”, preferably being encoded by a gene comprising the nucleic acid sequence SEQ ID NO. 103958, preferably the coding region thereof, conferred the production of or the increase in 3,4,5-trihydroxypentanoic acid compared with the wild type control.

The nucleic acid sequence of B0061 from Escherichia coli, e.g. as shown in the respective line in column 5 of Table I, application no. 17, has been published in B. And the activity of the gene product thereof is the activity of L-ribulose-5-phosphate 4-epimerase.

Accordingly, in one embodiment, the process of the present invention for producing raffinose in a non-human organism, like a microorganism or a plant or a part thereof, comprises increasing or generating the activity of a gene product with the activity of a gene product conferring the activity of “L-ribulose-5-phosphate 4-epimerase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said B0061, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0061, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0061, or a functional equivalent or a homolog thereof as depicted in column 8 of Table II, application no. 17, preferably a homolog or functional equivalent as depicted in column 8 of Table II B, application no. 17, and being depicted in the same respective line as said B0061, and preferably the activity is increased non-targeted, whereby the respective line disclose in column 7 the fine chemical raffinose.

Accordingly, in one embodiment, the molecule which activity is to be increased in the process of the invention is the gene product with an activity as a “L-ribulose-5-phosphate 4-epimerase”, preferably it is the molecule of section (a) or (b) of this paragraph.

In particular, it was observed that in plants, especially in Arabidopsis thaliana, increasing or generating the activity of a gene product non-targeted with the activity of a “L-ribulose-5-phosphate 4-epimerase”, preferably being encoded by a gene comprising the nucleic acid sequence SEQ ID NO. 35204, preferably the coding region thereof, conferred the production of or the increase in raffinose compared with the wild type control.

The nucleic acid sequence of B0124 from Escherichia coli, e.g. as shown in the respective line in column 5 of Table I, application no. 17, has been published in B. And the activity of the gene product thereof is the activity of glucose dehydrogenase.

Accordingly, in one embodiment, the process of the present invention for producing sucrose in a non-human organism, like a microorganism or a plant or a part thereof, comprises increasing or generating the activity of a gene product with the activity of a gene product conferring the activity of “glucose dehydrogenase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said B0124, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0124, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0124, or a functional equivalent or a homolog thereof as depicted in column 8 of Table II, application no. 17, preferably a homolog or functional equivalent as depicted in column 8 of Table II B, application no. 17, and being depicted in the same respective line as said B0124, and preferably the activity is increased non-targeted, whereby the respective line disclose in column 7 the fine chemical sucrose.

Accordingly, in one embodiment, the molecule which activity is to be increased in the process of the invention is the gene product with an activity as a “glucose dehydrogenase”, preferably it is the molecule of section (a) or (b) of this paragraph.

In particular, it was observed that in plants, especially in Arabidopsis thaliana, increasing or generating the activity of a gene product non-targeted with the activity of a “glucose dehydrogenase”, preferably being encoded by a gene comprising the nucleic acid sequence SEQ ID NO. 35482, preferably the coding region thereof, conferred the production of or the increase in sucrose compared with the wild type control.

The nucleic acid sequence of B0161 from Escherichia coli, e.g. as shown in the respective line in column 5 of Table I, application no. 17, has been published in B. And the activity of the gene product thereof is the activity of serine protease.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “serine protease”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B0161, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0161, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0161, or a functional equivalent or a homolog thereof as depicted in column 8 of Table II, application no. 17, preferably a homolog or functional equivalent as depicted in column 8 of Table II B, application no. 17, and being depicted in the same respective line as said B0161, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “serine protease”, preferably it is the molecule of section (a) or (b) of this paragraph.

In particular, it was observed that in plants, especially in Arabidopsis thaliana, increasing or generating the activity of a gene product non-targeted with the activity of a “serine protease”, preferably being encoded by a gene comprising the nucleic acid sequence SEQ ID NO. 7081, preferably the coding region thereof, conferred the production of or the increase in glucose-1-phosphate compared with the wild type control.

The nucleic acid sequence of B0161 from Escherichia coli, e.g. as shown in the respective line in column 5 of Table I, application no. 17, has been published in B. And the activity of the gene product thereof is the activity of serine protease.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “serine protease”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B0161, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0161, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0161, or a functional equivalent or a homolog thereof as depicted in column 8 of Table II, application no. 17, preferably a homolog or functional equivalent as depicted in column 8 of Table II B, application no. 17, and being depicted in the same respective line as said B0161, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “serine protease”, preferably it is the molecule of section (a) or (b) of this paragraph.

In particular, it was observed that in plants, especially in Arabidopsis thaliana, increasing or generating the activity of a gene product non-targeted with the activity of a “serine protease”, preferably being encoded by a gene comprising the nucleic acid sequence SEQ ID NO. 7081, preferably the coding region thereof, conferred the production of or the increase in myo-inositol compared with the wild type control.

The nucleic acid sequence of B0221 from Escherichia coli, e.g. as shown in the respective line in column 5 of Table I, application no. 17, has been published in B. And the activity of the gene product thereof is the activity of acyl-CoA dehydrogenase.

Accordingly, in one embodiment, the process of the present invention for producing sucrose in a non-human organism, like a microorganism or a plant or a part thereof, comprises increasing or generating the activity of a gene product with the activity of a gene product conferring the activity of “acyl-CoA dehydrogenase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said B0221, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0221, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0221, or a functional equivalent or a homolog thereof as depicted in column 8 of Table II, application no. 17, preferably a homolog or functional equivalent as depicted in column 8 of Table II B, application no. 17, and being depicted in the same respective line as said B0221, and preferably the activity is increased non-targeted, whereby the respective line disclose in column 7 the fine chemical sucrose.

Accordingly, in one embodiment, the molecule which activity is to be increased in the process of the invention is the gene product with an activity as a “acyl-CoA dehydrogenase”, preferably it is the molecule of section (a) or (b) of this paragraph.

In particular, it was observed that in plants, especially in Arabidopsis thaliana, increasing or generating the activity of a gene product non-targeted with the activity of a “acyl-CoA dehydrogenase”, preferably being encoded by a gene comprising the nucleic acid sequence SEQ ID NO. 35590, preferably the coding region thereof, conferred the production of or the increase in sucrose compared with the wild type control.

The nucleic acid sequence of B0251 from Escherichia coli, e.g. as shown in the respective line in column 5 of Table I, application no. 17, has been published in B. And the activity of the gene product thereof is the activity of b0251-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “b0251-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B0251, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0251, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0251, or a functional equivalent or a homolog thereof as depicted in column 8 of Table II, application no. 17, preferably a homolog or functional equivalent as depicted in column 8 of Table II B, application no. 17, and being depicted in the same respective line as said B0251, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “b0251-protein”, preferably it is the molecule of section (a) or (b) of this paragraph.

In particular, it was observed that in plants, especially in Arabidopsis thaliana, increasing or generating the activity of a gene product non-targeted with the activity of a “b0251-protein”, preferably being encoded by a gene comprising the nucleic acid sequence SEQ ID NO. 143742, preferably the coding region thereof, conferred the production of or the increase in glucose-1-phosphate compared with the wild type control.

The nucleic acid sequence of B0312 from Escherichia coli, e.g. as shown in the respective line in column 5 of Table I, application no. 17, has been published in B. And the activity of the gene product thereof is the activity of betaine aldehyde dehydrogenase.

Accordingly, in one embodiment, the process of the present invention for producing fructose in a non-human organism, like a microorganism or a plant or a part thereof, comprises increasing or generating the activity of a gene product with the activity of a gene product conferring the activity of “betaine aldehyde dehydrogenase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B0312, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0312, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0312, or a functional equivalent or a homolog thereof as depicted in column 8 of Table II, application no. 17, preferably a homolog or functional equivalent as depicted in column 8 of Table II B, application no. 17, and being depicted in the same respective line as said B0312, and preferably the activity is increased non-targeted, whereby the respective line disclose in column 7 the fine chemical fructose.

Accordingly, in one embodiment, the molecule which activity is to be increased in the process of the invention is the gene product with an activity as a “betaine aldehyde dehydrogenase”, preferably it is the molecule of section (a) or (b) of this paragraph.

In particular, it was observed that in plants, especially in Arabidopsis thaliana, increasing or generating the activity of a gene product non-targeted with the activity of a “betaine aldehyde dehydrogenase”, preferably being encoded by a gene comprising the nucleic acid sequence SEQ ID NO. 121111, preferably the coding region thereof, conferred the production of or the increase in fructose compared with the wild type control.

The nucleic acid sequence of B0344 from Escherichia coli, e.g. as shown in the respective line in column 5 of Table I, application no. 17, has been published in B. And the activity of the gene product thereof is the activity of beta-galactosidase.

Accordingly, in one embodiment, the process of the present invention for producing melibiose in a non-human organism, like a microorganism or a plant or a part thereof, comprises increasing or generating the activity of a gene product with the activity of a gene product conferring the activity of “beta-galactosidase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical melibiose), application no. 17, preferably the coding region thereof, or a homolog or a fragment thereof, and being depicted in the same respective line as said B0344, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0344, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0344, or a functional equivalent or a homolog thereof as depicted in column 8 of Table II, application no. 17, preferably a homolog or functional equivalent as depicted in column 8 of Table II B, application no. 17, and being depicted in the same respective line as said B0344, and preferably the activity is increased plastidic, whereby the respective line disclose in column 7 the fine chemical melibiose.

Accordingly, in one embodiment, the molecule which activity is to be increased in the process of the invention is the gene product with an activity as a “beta-galactosidase”, preferably it is the molecule of section (a) or (b) of this paragraph.

In particular, it was observed that in plants, especially in Arabidopsis thaliana, increasing or generating the activity of a gene product plastidic with the activity of a “beta-galactosidase”, preferably being encoded by a gene comprising the nucleic acid sequence SEQ ID NO. 35733, preferably the coding region thereof, conferred the production of or the increase in melibiose compared with the wild type control.

The nucleic acid sequence of B0362 from Escherichia coli, e.g. as shown in the respective line in column 5 of Table I, application no. 17, has been published in B. And the activity of the gene product thereof is the activity of b0362-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “b0362-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B0362, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0362, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0362, or a functional equivalent or a homolog thereof as depicted in column 8 of Table II, application no. 17, preferably a homolog or functional equivalent as depicted in column 8 of Table II B, application no. 17, and being depicted in the same respective line as said B0362, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “b0362-protein”, preferably it is the molecule of section (a) or (b) of this paragraph.

In particular, it was observed that in plants, especially in Arabidopsis thaliana, increasing or generating the activity of a gene product non-targeted with the activity of a “b0362-protein”, preferably being encoded by a gene comprising the nucleic acid sequence SEQ ID NO. 143761, preferably the coding region thereof, conferred the production of or the increase in glucose-1-phosphate compared with the wild type control.

The nucleic acid sequence of B0376 from Escherichia coli, e.g. as shown in the respective line in column 5 of Table I, application no. 17, has been published in B. And the activity of the gene product thereof is the activity of penicillin-binding protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “penicillin-binding protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B0376, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0376, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0376, or a functional equivalent or a homolog thereof as depicted in column 8 of Table II, application no. 17, preferably a homolog or functional equivalent as depicted in column 8 of Table II B, application no. 17, and being depicted in the same respective line as said B0376, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “penicillin-binding protein”, preferably it is the molecule of section (a) or (b) of this paragraph.

In particular, it was observed that in plants, especially in Arabidopsis thaliana, increasing or generating the activity of a gene product non-targeted with the activity of a “penicillin-binding protein”, preferably being encoded by a gene comprising the nucleic acid sequence SEQ ID NO. 143771, preferably the coding region thereof, conferred the production of or the increase in glucose-1-phosphate compared with the wild type control.

The nucleic acid sequence of B0449 from Escherichia coli, e.g. as shown in the respective line in column 5 of Table I, application no. 17, has been published in B. And the activity of the gene product thereof is the activity of ATP-binding component of a transport system.

Accordingly, in one embodiment, the process of the present invention for producing fructose in a non-human organism, like a microorganism or a plant or a part thereof, comprises increasing or generating the activity of a gene product with the activity of a gene product conferring the activity of “ATP-binding component of a transport system”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B0449, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0449, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0449, or a functional equivalent or a homolog thereof as depicted in column 8 of Table II, application no. 17, preferably a homolog or functional equivalent as depicted in column 8 of Table II B, application no. 17, and being depicted in the same respective line as said B0449, and preferably the activity is increased non-targeted, whereby the respective line disclose in column 7 the fine chemical fructose.

Accordingly, in one embodiment, the molecule which activity is to be increased in the process of the invention is the gene product with an activity as a “ATP-binding component of a transport system”, preferably it is the molecule of section (a) or (b) of this paragraph.

In particular, it was observed that in plants, especially in Arabidopsis thaliana, increasing or generating the activity of a gene product non-targeted with the activity of a “ATP-binding component of a transport system”, preferably being encoded by a gene comprising the nucleic acid sequence SEQ ID NO. 7333, preferably the coding region thereof, conferred the production of or the increase in fructose compared with the wild type control.

The nucleic acid sequence of B0449 from Escherichia coli, e.g. as shown in the respective line in column 5 of Table I, application no. 17, has been published in B. And the activity of the gene product thereof is the activity of ATP-binding component of a transport system.

Accordingly, in one embodiment, the process of the present invention for producing glucose in a non-human organism, like a microorganism or a plant or a part thereof, comprises increasing or generating the activity of a gene product with the activity of a gene product conferring the activity of “ATP-binding component of a transport system”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B0449, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0449, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0449, or a functional equivalent or a homolog thereof as depicted in column 8 of Table II, application no. 17, preferably a homolog or functional equivalent as depicted in column 8 of Table II B, application no. 17, and being depicted in the same respective line as said B0449, and preferably the activity is increased non-targeted, whereby the respective line disclose in column 7 the fine chemical glucose.

Accordingly, in one embodiment, the molecule which activity is to be increased in the process of the invention is the gene product with an activity as a “ATP-binding component of a transport system”, preferably it is the molecule of section (a) or (b) of this paragraph.

In particular, it was observed that in plants, especially in Arabidopsis thaliana, increasing or generating the activity of a gene product non-targeted with the activity of a “ATP-binding component of a transport system”, preferably being encoded by a gene comprising the nucleic acid sequence SEQ ID NO. 7333, preferably the coding region thereof, conferred the production of or the increase in glucose compared with the wild type control.

The nucleic acid sequence of B0486 from Escherichia coli, e.g. as shown in the respective line in column 5 of Table I, application no. 17, has been published in B. And the activity of the gene product thereof is the activity of membrane transport protein.

Accordingly, in one embodiment, the process of the present invention for producing glucose in a non-human organism, like a microorganism or a plant or a part thereof, comprises increasing or generating the activity of a gene product with the activity of a gene product conferring the activity of “membrane transport protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B0486, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0486, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0486, or a functional equivalent or a homolog thereof as depicted in column 8 of Table II, application no. 17, preferably a homolog or functional equivalent as depicted in column 8 of Table II B, application no. 17, and being depicted in the same respective line as said B0486, and preferably the activity is increased non-targeted, whereby the respective line disclose in column 7 the fine chemical glucose.

Accordingly, in one embodiment, the molecule which activity is to be increased in the process of the invention is the gene product with an activity as a “membrane transport protein”, preferably it is the molecule of section (a) or (b) of this paragraph.

In particular, it was observed that in plants, especially in Arabidopsis thaliana, increasing or generating the activity of a gene product non-targeted with the activity of a “membrane transport protein”, preferably being encoded by a gene comprising the nucleic acid sequence SEQ ID NO. 7686, preferably the coding region thereof, conferred the production of or the increase in glucose compared with the wild type control.

The nucleic acid sequence of B0488 from Escherichia coli, e.g. as shown in the respective line in column 5 of Table I, application no. 17, has been published in B. And the activity of the gene product thereof is the activity of b0488-protein.

Accordingly, in one embodiment, the process of the present invention for producing saccharic acid in a non-human organism, like a microorganism or a plant or a part thereof, comprises increasing or generating the activity of a gene product with the activity of a gene product conferring the activity of “b0488-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical saccharic acid), application no. 17, preferably the coding region thereof, or a homolog or a fragment thereof, and being depicted in the same respective line as said B0488, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0488, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0488, or a functional equivalent or a homolog thereof as depicted in column 8 of Table II, application no. 17, preferably a homolog or functional equivalent as depicted in column 8 of Table II B, application no. 17, and being depicted in the same respective line as said B0488, and preferably the activity is increased plastidic, whereby the respective line disclose in column 7 the fine chemical saccharic acid.

Accordingly, in one embodiment, the molecule which activity is to be increased in the process of the invention is the gene product with an activity as a “b0488-protein”, preferably it is the molecule of section (a) or (b) of this paragraph.

In particular, it was observed that in plants, especially in Arabidopsis thaliana, increasing or generating the activity of a gene product plastidic with the activity of a “b0488-protein”, preferably being encoded by a gene comprising the nucleic acid sequence SEQ ID NO. 143817, preferably the coding region thereof, conferred the production of or the increase in saccharic acid compared with the wild type control.

The nucleic acid sequence of B0514 from Escherichia coli, e.g. as shown in the respective line in column 5 of Table I, application no. 17, has been published in B. And the activity of the gene product thereof is the activity of glycerate kinase.

Accordingly, in one embodiment, the process of the present invention for producing galactinol in a non-human organism, like a microorganism or a plant or a part thereof, comprises increasing or generating the activity of a gene product with the activity of a gene product conferring the activity of “glycerate kinase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical galactinol), application no. 17, preferably the coding region thereof, or a homolog or a fragment thereof, and being depicted in the same respective line as said B0514, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0514, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0514, or a functional equivalent or a homolog thereof as depicted in column 8 of Table II, application no. 17, preferably a homolog or functional equivalent as depicted in column 8 of Table II B, application no. 17, and being depicted in the same respective line as said B0514, and preferably the activity is increased non-targeted, whereby the respective line disclose in column 7 the fine chemical galactinol.

Accordingly, in one embodiment, the molecule which activity is to be increased in the process of the invention is the gene product with an activity as a “glycerate kinase”, preferably it is the molecule of section (a) or (b) of this paragraph.

In particular, it was observed that in plants, especially in Arabidopsis thaliana, increasing or generating the activity of a gene product non-targeted with the activity of a “glycerate kinase”, preferably being encoded by a gene comprising the nucleic acid sequence SEQ ID NO. 134402, preferably the coding region thereof, conferred the production of or the increase in galactinol compared with the wild type control.

The nucleic acid sequence of B0593 from Escherichia coli, e.g. as shown in the respective line in column 5 of Table I, application no. 17, has been published in B. And the activity of the gene product thereof is the activity of isochorismate synthase.

Accordingly, in one embodiment, the process of the present invention for producing fructose in a non-human organism, like a microorganism or a plant or a part thereof, comprises increasing or generating the activity of a gene product with the activity of a gene product conferring the activity of “isochorismate synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B0593, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0593, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0593, or a functional equivalent or a homolog thereof as depicted in column 8 of Table II, application no. 17, preferably a homolog or functional equivalent as depicted in column 8 of Table II B, application no. 17, and being depicted in the same respective line as said B0593, and preferably the activity is increased plastidic, whereby the respective line disclose in column 7 the fine chemical fructose.

Accordingly, in one embodiment, the molecule which activity is to be increased in the process of the invention is the gene product with an activity as a “isochorismate synthase”, preferably it is the molecule of section (a) or (b) of this paragraph.

In particular, it was observed that in plants, especially in Arabidopsis thaliana, increasing or generating the activity of a gene product plastidic with the activity of a “isochorismate synthase”, preferably being encoded by a gene comprising the nucleic acid sequence SEQ ID NO. 35967, preferably the coding region thereof, conferred the production of or the increase in fructose compared with the wild type control.

The nucleic acid sequence of B0593 from Escherichia coli, e.g. as shown in the respective line in column 5 of Table I, application no. 17, has been published in B. And the activity of the gene product thereof is the activity of isochorismate synthase.

Accordingly, in one embodiment, the process of the present invention for producing glucose in a non-human organism, like a microorganism or a plant or a part thereof, comprises increasing or generating the activity of a gene product with the activity of a gene product conferring the activity of “isochorismate synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B0593, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0593, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0593, or a functional equivalent or a homolog thereof as depicted in column 8 of Table II, application no. 17, preferably a homolog or functional equivalent as depicted in column 8 of Table II B, application no. 17, and being depicted in the same respective line as said B0593, and preferably the activity is increased plastidic, whereby the respective line disclose in column 7 the fine chemical glucose.

Accordingly, in one embodiment, the molecule which activity is to be increased in the process of the invention is the gene product with an activity as a “isochorismate synthase”, preferably it is the molecule of section (a) or (b) of this paragraph.

In particular, it was observed that in plants, especially in Arabidopsis thaliana, increasing or generating the activity of a gene product plastidic with the activity of a “isochorismate synthase”, preferably being encoded by a gene comprising the nucleic acid sequence SEQ ID NO. 35967, preferably the coding region thereof, conferred the production of or the increase in glucose compared with the wild type control.

The nucleic acid sequence of B0593 from Escherichia coli, e.g. as shown in the respective line in column 5 of Table I, application no. 17, has been published in B. And the activity of the gene product thereof is the activity of isochorismate synthase.

Accordingly, in one embodiment, the process of the present invention for producing raffinose in a non-human organism, like a microorganism or a plant or a part thereof, comprises increasing or generating the activity of a gene product with the activity of a gene product conferring the activity of “isochorismate synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said B0593, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0593, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0593, or a functional equivalent or a homolog thereof as depicted in column 8 of Table II, application no. 17, preferably a homolog or functional equivalent as depicted in column 8 of Table II B, application no. 17, and being depicted in the same respective line as said B0593, and preferably the activity is increased plastidic, whereby the respective line disclose in column 7 the fine chemical raffinose.

Accordingly, in one embodiment, the molecule which activity is to be increased in the process of the invention is the gene product with an activity as a “isochorismate synthase”, preferably it is the molecule of section (a) or (b) of this paragraph.

In particular, it was observed that in plants, especially in Arabidopsis thaliana, increasing or generating the activity of a gene product plastidic with the activity of a “isochorismate synthase”, preferably being encoded by a gene comprising the nucleic acid sequence SEQ ID NO. 35967, preferably the coding region thereof, conferred the production of or the increase in raffinose compared with the wild type control.

The nucleic acid sequence of B0593 from Escherichia coli, e.g. as shown in the respective line in column 5 of Table I, application no. 17, has been published in B. And the activity of the gene product thereof is the activity of isochorismate synthase.

Accordingly, in one embodiment, the process of the present invention for producing sucrose in a non-human organism, like a microorganism or a plant or a part thereof, comprises increasing or generating the activity of a gene product with the activity of a gene product conferring the activity of “isochorismate synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said B0593, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0593, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0593, or a functional equivalent or a homolog thereof as depicted in column 8 of Table II, application no. 17, preferably a homolog or functional equivalent as depicted in column 8 of Table II B, application no. 17, and being depicted in the same respective line as said B0593, and preferably the activity is increased plastidic, whereby the respective line disclose in column 7 the fine chemical sucrose.

Accordingly, in one embodiment, the molecule which activity is to be increased in the process of the invention is the gene product with an activity as a “isochorismate synthase”, preferably it is the molecule of section (a) or (b) of this paragraph.

In particular, it was observed that in plants, especially in Arabidopsis thaliana, increasing or generating the activity of a gene product plastidic with the activity of a “isochorismate synthase”, preferably being encoded by a gene comprising the nucleic acid sequence SEQ ID NO. 35967, preferably the coding region thereof, conferred the production of or the increase in sucrose compared with the wild type control.

The nucleic acid sequence of B0644 from Escherichia coli, e.g. as shown in the respective line in column 5 of Table I, application no. 17, has been published in B. And the activity of the gene product thereof is the activity of B0644-protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “B0644-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B0644, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0644, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0644, or a functional equivalent or a homolog thereof as depicted in column 8 of Table II, application no. 17, preferably a homolog or functional equivalent as depicted in column 8 of Table II B, application no. 17, and being depicted in the same respective line as said B0644, and preferably the activity is increased plastidic, whereby the respective line disclose in column 7 the fine chemical 3,4,5-trihydroxypentanoic acid.

Accordingly, in one embodiment, the molecule which activity is to be increased in the process of the invention is the gene product with an activity as a “B0644-protein”, preferably it is the molecule of section (a) or (b) of this paragraph.

In particular, it was observed that in plants, especially in Arabidopsis thaliana, increasing or generating the activity of a gene product plastidic with the activity of a “B0644-protein”, preferably being encoded by a gene comprising the nucleic acid sequence SEQ ID NO. 143901, preferably the coding region thereof, conferred the production of or the increase in 3,4,5-trihydroxypentanoic acid compared with the wild type control.

The nucleic acid sequence of B0651 from Escherichia coli, e.g. as shown in the respective line in column 5 of Table I, application no. 17, has been published in B. And the activity of the gene product thereof is the activity of pyrimidine-specific ribonucleoside hydrolase.

Accordingly, in one embodiment, the process of the present invention for producing saccharic acid in a non-human organism, like a microorganism or a plant or a part thereof, comprises increasing or generating the activity of a gene product with the activity of a gene product conferring the activity of “pyrimidine-specific ribonucleoside hydrolase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical saccharic acid), application no. 17, preferably the coding region thereof, or a homolog or a fragment thereof, and being depicted in the same respective line as said B0651, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0651, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0651, or a functional equivalent or a homolog thereof as depicted in column 8 of Table II, application no. 17, preferably a homolog or functional equivalent as depicted in column 8 of Table II B, application no. 17, and being depicted in the same respective line as said B0651, and preferably the activity is increased non-targeted, whereby the respective line disclose in column 7 the fine chemical saccharic acid.

Accordingly, in one embodiment, the molecule which activity is to be increased in the process of the invention is the gene product with an activity as a “pyrimidine-specific ribonucleoside hydrolase”, preferably it is the molecule of section (a) or (b) of this paragraph.

In particular, it was observed that in plants, especially in Arabidopsis thaliana, increasing or generating the activity of a gene product non-targeted with the activity of a “pyrimidine-specific ribonucleoside hydrolase”, preferably being encoded by a gene comprising the nucleic acid sequence SEQ ID NO. 143954, preferably the coding region thereof, conferred the production of or the increase in saccharic acid compared with the wild type control.

The nucleic acid sequence of B0674 from Escherichia coli, e.g. as shown in the respective line in column 5 of Table I, application no. 17, has been published in B. And the activity of the gene product thereof is the activity of asparagine synthetase B.

Accordingly, in one embodiment, the process of the present invention for producing erythrol in a non-human organism, like a microorganism or a plant or a part thereof, comprises increasing or generating the activity of a gene product with the activity of a gene product conferring the activity of “asparagine synthetase B”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical erythrol), application no. 17, preferably the coding region thereof, or a homolog or a fragment thereof, and being depicted in the same respective line as said B0674, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0674, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0674, or a functional equivalent or a homolog thereof as depicted in column 8 of Table II, application no. 17, preferably a homolog or functional equivalent as depicted in column 8 of Table II B, application no. 17, and being depicted in the same respective line as said B0674, and preferably the activity is increased plastidic, whereby the respective line disclose in column 7 the fine chemical erythrol.

Accordingly, in one embodiment, the molecule which activity is to be increased in the process of the invention is the gene product with an activity as a “asparagine synthetase B”, preferably it is the molecule of section (a) or (b) of this paragraph.

In particular, it was observed that in plants, especially in Arabidopsis thaliana, increasing or generating the activity of a gene product plastidic with the activity of a “asparagine synthetase B”, preferably being encoded by a gene comprising the nucleic acid sequence SEQ ID NO. 144207, preferably the coding region thereof, conferred the production of or the increase in erythrol compared with the wild type control.

The nucleic acid sequence of B0695 from Escherichia coli, e.g. as shown in the respective line in column 5 of Table I, application no. 17, has been published in B. And the activity of the gene product thereof is the activity of sensor histidine kinase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “sensor histidine kinase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B0695, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0695, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0695, or a functional equivalent or a homolog thereof as depicted in column 8 of Table II, application no. 17, preferably a homolog or functional equivalent as depicted in column 8 of Table II B, application no. 17, and being depicted in the same respective line as said B0695, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “sensor histidine kinase”, preferably it is the molecule of section (a) or (b) of this paragraph.

In particular, it was observed that in plants, especially in Arabidopsis thaliana, increasing or generating the activity of a gene product non-targeted with the activity of a “sensor histidine kinase”, preferably being encoded by a gene comprising the nucleic acid sequence SEQ ID NO. 93678, preferably the coding region thereof, conferred the production of or the increase in myo-inositol compared with the wild type control.

The nucleic acid sequence of B0728 from Escherichia coli, e.g. as shown in the respective line in column 5 of Table I, application no. 17, has been published in B. And the activity of the gene product thereof is the activity of succinyl-CoA synthetase beta chain.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “succinyl-CoA synthetase beta chain”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B0728, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0728, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0728, or a functional equivalent or a homolog thereof as depicted in column 8 of Table II, application no. 17, preferably a homolog or functional equivalent as depicted in column 8 of Table II B, application no. 17, and being depicted in the same respective line as said B0728, and preferably the activity is increased plastidic, whereby the respective line disclose in column 7 the fine chemical myo-inositol.

Accordingly, in one embodiment, the molecule which activity is to be increased in the process of the invention is the gene product with an activity as a “succinyl-CoA synthetase beta chain”, preferably it is the molecule of section (a) or (b) of this paragraph.

In particular, it was observed that in plants, especially in Arabidopsis thaliana, increasing or generating the activity of a gene product plastidic with the activity of a “succinyl-CoA synthetase beta chain”, preferably being encoded by a gene comprising the nucleic acid sequence SEQ ID NO. 93793, preferably the coding region thereof, conferred the production of or the increase in myo-inositol compared with the wild type control.

The nucleic acid sequence of B0752 from Escherichia coli, e.g. as shown in the respective line in column 5 of Table I, application no. 17, has been published in B. And the activity of the gene product thereof is the activity of zinc transporter.

Accordingly, in one embodiment, the process of the present invention for producing fructose in a non-human organism, like a microorganism or a plant or a part thereof, comprises increasing or generating the activity of a gene product with the activity of a gene product conferring the activity of “zinc transporter”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B0752, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0752, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0752, or a functional equivalent or a homolog thereof as depicted in column 8 of Table II, application no. 17, preferably a homolog or functional equivalent as depicted in column 8 of Table II B, application no. 17, and being depicted in the same respective line as said B0752, and preferably the activity is increased non-targeted, whereby the respective line disclose in column 7 the fine chemical fructose.

Accordingly, in one embodiment, the molecule which activity is to be increased in the process of the invention is the gene product with an activity as a “zinc transporter”, preferably it is the molecule of section (a) or (b) of this paragraph.

In particular, it was observed that in plants, especially in Arabidopsis thaliana, increasing or generating the activity of a gene product non-targeted with the activity of a “zinc transporter”, preferably being encoded by a gene comprising the nucleic acid sequence SEQ ID NO. 36114, preferably the coding region thereof, conferred the production of or the increase in fructose compared with the wild type control.

The nucleic acid sequence of B0752 from Escherichia coli, e.g. as shown in the respective line in column 5 of Table I, application no. 17, has been published in B. And the activity of the gene product thereof is the activity of zinc transporter.

Accordingly, in one embodiment, the process of the present invention for producing glucose in a non-human organism, like a microorganism or a plant or a part thereof, comprises increasing or generating the activity of a gene product with the activity of a gene product conferring the activity of “zinc transporter”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B0752, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0752, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0752, or a functional equivalent or a homolog thereof as depicted in column 8 of Table II, application no. 17, preferably a homolog or functional equivalent as depicted in column 8 of Table II B, application no. 17, and being depicted in the same respective line as said B0752, and preferably the activity is increased non-targeted, whereby the respective line disclose in column 7 the fine chemical glucose.

Accordingly, in one embodiment, the molecule which activity is to be increased in the process of the invention is the gene product with an activity as a “zinc transporter”, preferably it is the molecule of section (a) or (b) of this paragraph.

In particular, it was observed that in plants, especially in Arabidopsis thaliana, increasing or generating the activity of a gene product non-targeted with the activity of a “zinc transporter”, preferably being encoded by a gene comprising the nucleic acid sequence SEQ ID NO. 36114, preferably the coding region thereof, conferred the production of or the increase in glucose compared with the wild type control.

The nucleic acid sequence of B0752 from Escherichia coli, e.g. as shown in the respective line in column 5 of Table I, application no. 17, has been published in B. And the activity of the gene product thereof is the activity of zinc transporter.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “zinc transporter”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B0752, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0752, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0752, or a functional equivalent or a homolog thereof as depicted in column 8 of Table II, application no. 17, preferably a homolog or functional equivalent as depicted in column 8 of Table II B, application no. 17, and being depicted in the same respective line as said B0752, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “zinc transporter”, preferably it is the molecule of section (a) or (b) of this paragraph.

In particular, it was observed that in plants, especially in Arabidopsis thaliana, increasing or generating the activity of a gene product non-targeted with the activity of a “zinc transporter”, preferably being encoded by a gene comprising the nucleic acid sequence SEQ ID NO. 36114, preferably the coding region thereof, conferred the production of or the increase in glucose-1-phosphate compared with the wild type control.

The nucleic acid sequence of B0752 from Escherichia coli, e.g. as shown in the respective line in column 5 of Table I, application no. 17, has been published in B. And the activity of the gene product thereof is the activity of zinc transporter.

Accordingly, in one embodiment, the process of the present invention for producing raffinose in a non-human organism, like a microorganism or a plant or a part thereof, comprises increasing or generating the activity of a gene product with the activity of a gene product conferring the activity of “zinc transporter”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said B0752, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0752, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0752, or a functional equivalent or a homolog thereof as depicted in column 8 of Table II, application no. 17, preferably a homolog or functional equivalent as depicted in column 8 of Table II B, application no. 17, and being depicted in the same respective line as said B0752, and preferably the activity is increased non-targeted, whereby the respective line disclose in column 7 the fine chemical raffinose.

Accordingly, in one embodiment, the molecule which activity is to be increased in the process of the invention is the gene product with an activity as a “zinc transporter”, preferably it is the molecule of section (a) or (b) of this paragraph.

In particular, it was observed that in plants, especially in Arabidopsis thaliana, increasing or generating the activity of a gene product non-targeted with the activity of a “zinc transporter”, preferably being encoded by a gene comprising the nucleic acid sequence SEQ ID NO. 36114, preferably the coding region thereof, conferred the production of or the increase in raffinose compared with the wild type control.

The nucleic acid sequence of B0801 from Escherichia coli, e.g. as shown in the respective line in column 5 of Table I, application no. 17, has been published in B. And the activity of the gene product thereof is the activity of b0801-protein.

Accordingly, in one embodiment, the process of the present invention for producing glucose in a non-human organism, like a microorganism or a plant or a part thereof, comprises increasing or generating the activity of a gene product with the activity of a gene product conferring the activity of “b0801-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B0801, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0801, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0801, or a functional equivalent or a homolog thereof as depicted in column 8 of Table II, application no. 17, preferably a homolog or functional equivalent as depicted in column 8 of Table II B, application no. 17, and being depicted in the same respective line as said B0801, and preferably the activity is increased non-targeted, whereby the respective line disclose in column 7 the fine chemical glucose.

Accordingly, in one embodiment, the molecule which activity is to be increased in the process of the invention is the gene product with an activity as a “b0801-protein”, preferably it is the molecule of section (a) or (b) of this paragraph.

In particular, it was observed that in plants, especially in Arabidopsis thaliana, increasing or generating the activity of a gene product non-targeted with the activity of a “b0801-protein”, preferably being encoded by a gene comprising the nucleic acid sequence SEQ ID NO. 75807, preferably the coding region thereof, conferred the production of or the increase in glucose compared with the wild type control.

The nucleic acid sequence of B0856 from Escherichia coli, e.g. as shown in the respective line in column 5 of Table I, application no. 17, has been published in B. And the activity of the gene product thereof is the activity of putrescine transport system permease protein.

Accordingly, in one embodiment, the process of the present invention for producing sedoheptulose-7-phosphate in a non-human organism, like a microorganism or a plant or a part thereof, comprises increasing or generating the activity of a gene product with the activity of a gene product conferring the activity of “putrescine transport system permease protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical sedoheptulose-7-phosphate), application no. 17, preferably the coding region thereof, or a homolog or a fragment thereof, and being depicted in the same respective line as said B0856, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0856, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0856, or a functional equivalent or a homolog thereof as depicted in column 8 of Table II, application no. 17, preferably a homolog or functional equivalent as depicted in column 8 of Table II B, application no. 17, and being depicted in the same respective line as said B0856, and preferably the activity is increased plastidic, whereby the respective line disclose in column 7 the fine chemical sedoheptulose-7-phosphate.

Accordingly, in one embodiment, the molecule which activity is to be increased in the process of the invention is the gene product with an activity as a “putrescine transport system permease protein”, preferably it is the molecule of section (a) or (b) of this paragraph.

In particular, it was observed that in plants, especially in Arabidopsis thaliana, increasing or generating the activity of a gene product plastidic with the activity of a “putrescine transport system permease protein”, preferably being encoded by a gene comprising the nucleic acid sequence SEQ ID NO. 131359, preferably the coding region thereof, conferred the production of or the increase in sedoheptulose-7-phosphate compared with the wild type control.

The nucleic acid sequence of B0885 from Escherichia coli, e.g. as shown in the respective line in column 5 of Table I, application no. 17, has been published in B. And the activity of the gene product thereof is the activity of leucyl/phenylalanyl-tRNA-protein transferase.

Accordingly, in one embodiment, the process of the present invention for producing raffinose in a non-human organism, like a microorganism or a plant or a part thereof, comprises increasing or generating the activity of a gene product with the activity of a gene product conferring the activity of “leucyl/phenylalanyl-tRNA-protein transferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said B0885, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0885, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0885, or a functional equivalent or a homolog thereof as depicted in column 8 of Table II, application no. 17, preferably a homolog or functional equivalent as depicted in column 8 of Table II B, application no. 17, and being depicted in the same respective line as said B0885, and preferably the activity is increased non-targeted, whereby the respective line disclose in column 7 the fine chemical raffinose.

Accordingly, in one embodiment, the molecule which activity is to be increased in the process of the invention is the gene product with an activity as a “leucyl/phenylalanyl-tRNA-protein transferase”, preferably it is the molecule of section (a) or (b) of this paragraph.

In particular, it was observed that in plants, especially in Arabidopsis thaliana, increasing or generating the activity of a gene product non-targeted with the activity of a “leucyl/phenylalanyl-tRNA-protein transferase”, preferably being encoded by a gene comprising the nucleic acid sequence SEQ ID NO. 36489, preferably the coding region thereof, conferred the production of or the increase in raffinose compared with the wild type control.

The nucleic acid sequence of B0970 from Escherichia coli, e.g. as shown in the respective line in column 5 of Table I, application no. 17, has been published in B. And the activity of the gene product thereof is the activity of b0970-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “b0970-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B0970, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0970, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0970, or a functional equivalent or a homolog thereof as depicted in column 8 of Table II, application no. 17, preferably a homolog or functional equivalent as depicted in column 8 of Table II B, application no. 17, and being depicted in the same respective line as said B0970, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “b0970-protein”, preferably it is the molecule of section (a) or (b) of this paragraph.

In particular, it was observed that in plants, especially in Arabidopsis thaliana, increasing or generating the activity of a gene product non-targeted with the activity of a “b0970-protein”, preferably being encoded by a gene comprising the nucleic acid sequence SEQ ID NO. 144479, preferably the coding region thereof, conferred the production of or the increase in glucose-1-phosphate compared with the wild type control.

The nucleic acid sequence of B0980 from Escherichia coli, e.g. as shown in the respective line in column 5 of Table I, application no. 17, has been published in B. And the activity of the gene product thereof is the activity of phosphoanhydride phosphorylase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “phosphoanhydride phosphorylase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B0980, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B0980, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B0980, or a functional equivalent or a homolog thereof as depicted in column 8 of Table II, application no. 17, preferably a homolog or functional equivalent as depicted in column 8 of Table II B, application no. 17, and being depicted in the same respective line as said B0980, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “phosphoanhydride phosphorylase”, preferably it is the molecule of section (a) or (b) of this paragraph.

In particular, it was observed that in plants, especially in Arabidopsis thaliana, increasing or generating the activity of a gene product non-targeted with the activity of a “phosphoanhydride phosphorylase”, preferably being encoded by a gene comprising the nucleic acid sequence SEQ ID NO. 36809, preferably the coding region thereof, conferred the production of or the increase in myo-inositol compared with the wild type control.

The nucleic acid sequence of B1003 from Escherichia coli, e.g. as shown in the respective line in column 5 of Table I, application no. 17, has been published in B. And the activity of the gene product thereof is the activity of b1003-protein.

Accordingly, in one embodiment, the process of the present invention for producing fructose in a non-human organism, like a microorganism or a plant or a part thereof, comprises increasing or generating the activity of a gene product with the activity of a gene product conferring the activity of “b1003-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B1003, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1003, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1003, or a functional equivalent or a homolog thereof as depicted in column 8 of Table II, application no. 17, preferably a homolog or functional equivalent as depicted in column 8 of Table II B, application no. 17, and being depicted in the same respective line as said B1003, and preferably the activity is increased non-targeted, whereby the respective line disclose in column 7 the fine chemical fructose.

Accordingly, in one embodiment, the molecule which activity is to be increased in the process of the invention is the gene product with an activity as a “b1003-protein”, preferably it is the molecule of section (a) or (b) of this paragraph.

In particular, it was observed that in plants, especially in Arabidopsis thaliana, increasing or generating the activity of a gene product non-targeted with the activity of a “b1003-protein”, preferably being encoded by a gene comprising the nucleic acid sequence SEQ ID NO. 7941, preferably the coding region thereof, conferred the production of or the increase in fructose compared with the wild type control.

The nucleic acid sequence of B1003 from Escherichia coli, e.g. as shown in the respective line in column 5 of Table I, application no. 17, has been published in B. And the activity of the gene product thereof is the activity of b1003-protein.

Accordingly, in one embodiment, the process of the present invention for producing glucose in a non-human organism, like a microorganism or a plant or a part thereof, comprises increasing or generating the activity of a gene product with the activity of a gene product conferring the activity of “b1003-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B1003, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1003, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1003, or a functional equivalent or a homolog thereof as depicted in column 8 of Table II, application no. 17, preferably a homolog or functional equivalent as depicted in column 8 of Table II B, application no. 17, and being depicted in the same respective line as said B1003, and preferably the activity is increased non-targeted, whereby the respective line disclose in column 7 the fine chemical glucose.

Accordingly, in one embodiment, the molecule which activity is to be increased in the process of the invention is the gene product with an activity as a “b1003-protein”, preferably it is the molecule of section (a) or (b) of this paragraph.

In particular, it was observed that in plants, especially in Arabidopsis thaliana, increasing or generating the activity of a gene product non-targeted with the activity of a “b1003-protein”, preferably being encoded by a gene comprising the nucleic acid sequence SEQ ID NO. 7941, preferably the coding region thereof, conferred the production of or the increase in glucose compared with the wild type control.

The nucleic acid sequence of B1003 from Escherichia coli, e.g. as shown in the respective line in column 5 of Table I, application no. 17, has been published in B. And the activity of the gene product thereof is the activity of b1003-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “b1003-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B1003, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1003, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1003, or a functional equivalent or a homolog thereof as depicted in column 8 of Table II, application no. 17, preferably a homolog or functional equivalent as depicted in column 8 of Table II B, application no. 17, and being depicted in the same respective line as said B1003, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “b1003-protein”, preferably it is the molecule of section (a) or (b) of this paragraph.

In particular, it was observed that in plants, especially in Arabidopsis thaliana, increasing or generating the activity of a gene product non-targeted with the activity of a “b1003-protein”, preferably being encoded by a gene comprising the nucleic acid sequence SEQ ID NO. 7941, preferably the coding region thereof, conferred the production of or the increase in glucose-1-phosphate compared with the wild type control.

The nucleic acid sequence of B1003 from Escherichia coli, e.g. as shown in the respective line in column 5 of Table I, application no. 17, has been published in B. And the activity of the gene product thereof is the activity of b1003-protein.

Accordingly, in one embodiment, the process of the present invention for producing sedoheptulose-7-phosphate in a non-human organism, like a microorganism or a plant or a part thereof, comprises increasing or generating the activity of a gene product with the activity of a gene product conferring the activity of “b1003-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical sedoheptulose-7-phosphate), application no. 17, preferably the coding region thereof, or a homolog or a fragment thereof, and being depicted in the same respective line as said B1003, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1003, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1003, or a functional equivalent or a homolog thereof as depicted in column 8 of Table II, application no. 17, preferably a homolog or functional equivalent as depicted in column 8 of Table II B, application no. 17, and being depicted in the same respective line as said B1003, and preferably the activity is increased non-targeted, whereby the respective line disclose in column 7 the fine chemical sedoheptulose-7-phosphate.

Accordingly, in one embodiment, the molecule which activity is to be increased in the process of the invention is the gene product with an activity as a “b1003-protein”, preferably it is the molecule of section (a) or (b) of this paragraph.

In particular, it was observed that in plants, especially in Arabidopsis thaliana, increasing or generating the activity of a gene product non-targeted with the activity of a “b1003-protein”, preferably being encoded by a gene comprising the nucleic acid sequence SEQ ID NO. 7941, preferably the coding region thereof, conferred the production of or the increase in sedoheptulose-7-phosphate compared with the wild type control.

The nucleic acid sequence of B1003 from Escherichia coli, e.g. as shown in the respective line in column 5 of Table I, application no. 17, has been published in B. And the activity of the gene product thereof is the activity of b1003-protein.

Accordingly, in one embodiment, the process of the present invention for producing sucrose in a non-human organism, like a microorganism or a plant or a part thereof, comprises increasing or generating the activity of a gene product with the activity of a gene product conferring the activity of “b1003-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said B1003, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1003, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1003, or a functional equivalent or a homolog thereof as depicted in column 8 of Table II, application no. 17, preferably a homolog or functional equivalent as depicted in column 8 of Table II B, application no. 17, and being depicted in the same respective line as said B1003, and preferably the activity is increased non-targeted, whereby the respective line disclose in column 7 the fine chemical sucrose.

Accordingly, in one embodiment, the molecule which activity is to be increased in the process of the invention is the gene product with an activity as a “b1003-protein”, preferably it is the molecule of section (a) or (b) of this paragraph.

In particular, it was observed that in plants, especially in Arabidopsis thaliana, increasing or generating the activity of a gene product non-targeted with the activity of a “b1003-protein”, preferably being encoded by a gene comprising the nucleic acid sequence SEQ ID NO. 7941, preferably the coding region thereof, conferred the production of or the increase in sucrose compared with the wild type control.

The nucleic acid sequence of B1046 from Escherichia coli, e.g. as shown in the respective line in column 5 of Table I, application no. 17, has been published in B. And the activity of the gene product thereof is the activity of b1046-protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “b1046-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B1046, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1046, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1046, or a functional equivalent or a homolog thereof as depicted in column 8 of Table II, application no. 17, preferably a homolog or functional equivalent as depicted in column 8 of Table II B, application no. 17, and being depicted in the same respective line as said B1046, and preferably the activity is increased plastidic, whereby the respective line disclose in column 7 the fine chemical 3,4,5-trihydroxypentanoic acid.

Accordingly, in one embodiment, the molecule which activity is to be increased in the process of the invention is the gene product with an activity as a “b1046-protein”, preferably it is the molecule of section (a) or (b) of this paragraph.

In particular, it was observed that in plants, especially in Arabidopsis thaliana, increasing or generating the activity of a gene product plastidic with the activity of a “b1046-protein”, preferably being encoded by a gene comprising the nucleic acid sequence SEQ ID NO. 124051, preferably the coding region thereof, conferred the production of or the increase in 3,4,5-trihydroxypentanoic acid compared with the wild type control.

The nucleic acid sequence of B1065 from Escherichia coli, e.g. as shown in the respective line in column 5 of Table I, application no. 17, has been published in B. And the activity of the gene product thereof is the activity of multidrug resistance protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “multidrug resistance protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B1065, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1065, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1065, or a functional equivalent or a homolog thereof as depicted in column 8 of Table II, application no. 17, preferably a homolog or functional equivalent as depicted in column 8 of Table II B, application no. 17, and being depicted in the same respective line as said B1065, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “multidrug resistance protein”, preferably it is the molecule of section (a) or (b) of this paragraph.

In particular, it was observed that in plants, especially in Arabidopsis thaliana, increasing or generating the activity of a gene product plastidic with the activity of a “multidrug resistance protein”, preferably being encoded by a gene comprising the nucleic acid sequence SEQ ID NO. 76032, preferably the coding region thereof, conferred the production of or the increase in glucose-1-phosphate compared with the wild type control.

The nucleic acid sequence of B1065 from Escherichia coli, e.g. as shown in the respective line in column 5 of Table I, application no. 17, has been published in B. And the activity of the gene product thereof is the activity of multidrug resistance protein.

Accordingly, in one embodiment, the process of the present invention for producing sucrose in a non-human organism, like a microorganism or a plant or a part thereof, comprises increasing or generating the activity of a gene product with the activity of a gene product conferring the activity of “multidrug resistance protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said B1065, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1065, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1065, or a functional equivalent or a homolog thereof as depicted in column 8 of Table II, application no. 17, preferably a homolog or functional equivalent as depicted in column 8 of Table II B, application no. 17, and being depicted in the same respective line as said B1065, and preferably the activity is increased plastidic, whereby the respective line disclose in column 7 the fine chemical sucrose.

Accordingly, in one embodiment, the molecule which activity is to be increased in the process of the invention is the gene product with an activity as a “multidrug resistance protein”, preferably it is the molecule of section (a) or (b) of this paragraph.

In particular, it was observed that in plants, especially in Arabidopsis thaliana, increasing or generating the activity of a gene product plastidic with the activity of a “multidrug resistance protein”, preferably being encoded by a gene comprising the nucleic acid sequence SEQ ID NO. 76032, preferably the coding region thereof, conferred the production of or the increase in sucrose compared with the wild type control.

The nucleic acid sequence of B1102 from Escherichia coli, e.g. as shown in the respective line in column 5 of Table I, application no. 17, has been published in B. And the activity of the gene product thereof is the activity of outer membrane receptor protein precursor.

Accordingly, in one embodiment, the process of the present invention for producing fructose in a non-human organism, like a microorganism or a plant or a part thereof, comprises increasing or generating the activity of a gene product with the activity of a gene product conferring the activity of “outer membrane receptor protein precursor”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B1102, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1102, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1102, or a functional equivalent or a homolog thereof as depicted in column 8 of Table II, application no. 17, preferably a homolog or functional equivalent as depicted in column 8 of Table II B, application no. 17, and being depicted in the same respective line as said B1102, and preferably the activity is increased non-targeted, whereby the respective line disclose in column 7 the fine chemical fructose.

Accordingly, in one embodiment, the molecule which activity is to be increased in the process of the invention is the gene product with an activity as a “outer membrane receptor protein precursor”, preferably it is the molecule of section (a) or (b) of this paragraph.

In particular, it was observed that in plants, especially in Arabidopsis thaliana, increasing or generating the activity of a gene product non-targeted with the activity of a “outer membrane receptor protein precursor”, preferably being encoded by a gene comprising the nucleic acid sequence SEQ ID NO. 114979, preferably the coding region thereof, conferred the production of or the increase in fructose compared with the wild type control.

The nucleic acid sequence of B1131 from Escherichia coli, e.g. as shown in the respective line in column 5 of Table I, application no. 17, has been published in B. And the activity of the gene product thereof is the activity of adenylosuccinate lyase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “adenylosuccinate lyase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B1131, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1131, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1131, or a functional equivalent or a homolog thereof as depicted in column 8 of Table II, application no. 17, preferably a homolog or functional equivalent as depicted in column 8 of Table II B, application no. 17, and being depicted in the same respective line as said B1131, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “adenylosuccinate lyase”, preferably it is the molecule of section (a) or (b) of this paragraph.

In particular, it was observed that in plants, especially in Arabidopsis thaliana, increasing or generating the activity of a gene product plastidic with the activity of a “adenylosuccinate lyase”, preferably being encoded by a gene comprising the nucleic acid sequence SEQ ID NO. 144611, preferably the coding region thereof, conferred the production of or the increase in glucose-1-phosphate compared with the wild type control.

The nucleic acid sequence of B1136 from Escherichia coli, e.g. as shown in the respective line in column 5 of Table I, application no. 17, has been published in B. And the activity of the gene product thereof is the activity of isocitrate dehydrogenase.

Accordingly, in one embodiment, the process of the present invention for producing glucose in a non-human organism, like a microorganism or a plant or a part thereof, comprises increasing or generating the activity of a gene product with the activity of a gene product conferring the activity of “isocitrate dehydrogenase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B1136, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1136, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1136, or a functional equivalent or a homolog thereof as depicted in column 8 of Table II, application no. 17, preferably a homolog or functional equivalent as depicted in column 8 of Table II B, application no. 17, and being depicted in the same respective line as said B1136, and preferably the activity is increased plastidic, whereby the respective line disclose in column 7 the fine chemical glucose.

Accordingly, in one embodiment, the molecule which activity is to be increased in the process of the invention is the gene product with an activity as a “isocitrate dehydrogenase”, preferably it is the molecule of section (a) or (b) of this paragraph.

In particular, it was observed that in plants, especially in Arabidopsis thaliana, increasing or generating the activity of a gene product plastidic with the activity of a “isocitrate dehydrogenase”, preferably being encoded by a gene comprising the nucleic acid sequence SEQ ID NO. 36971, preferably the coding region thereof, conferred the production of or the increase in glucose compared with the wild type control.

The nucleic acid sequence of B1137 from Escherichia coli, e.g. as shown in the respective line in column 5 of Table I, application no. 17, has been published in B. And the activity of the gene product thereof is the activity of b1137-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “b1137-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B1137, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1137, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1137, or a functional equivalent or a homolog thereof as depicted in column 8 of Table II, application no. 17, preferably a homolog or functional equivalent as depicted in column 8 of Table II B, application no. 17, and being depicted in the same respective line as said B1137, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “b1137-protein”, preferably it is the molecule of section (a) or (b) of this paragraph.

In particular, it was observed that in plants, especially in Arabidopsis thaliana, increasing or generating the activity of a gene product non-targeted with the activity of a “b1137-protein”, preferably being encoded by a gene comprising the nucleic acid sequence SEQ ID NO. 37390, preferably the coding region thereof, conferred the production of or the increase in glucose-1-phosphate compared with the wild type control.

The nucleic acid sequence of B1186 from Escherichia coli, e.g. as shown in the respective line in column 5 of Table I, application no. 17, has been published in B. And the activity of the gene product thereof is the activity of sodium/proton antiporter.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “sodium/proton antiporter”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B1186, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1186, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1186, or a functional equivalent or a homolog thereof as depicted in column 8 of Table II, application no. 17, preferably a homolog or functional equivalent as depicted in column 8 of Table II B, application no. 17, and being depicted in the same respective line as said B1186, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “sodium/proton antiporter”, preferably it is the molecule of section (a) or (b) of this paragraph.

In particular, it was observed that in plants, especially in Arabidopsis thaliana, increasing or generating the activity of a gene product non-targeted with the activity of a “sodium/proton antiporter”, preferably being encoded by a gene comprising the nucleic acid sequence SEQ ID NO. 37400, preferably the coding region thereof, conferred the production of or the increase in glucose-1-phosphate compared with the wild type control.

The nucleic acid sequence of B1214 from Escherichia coli, e.g. as shown in the respective line in column 5 of Table I, application no. 17, has been published in B. And the activity of the gene product thereof is the activity of b1214-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “b1214-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B1214, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1214, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1214, or a functional equivalent or a homolog thereof as depicted in column 8 of Table II, application no. 17, preferably a homolog or functional equivalent as depicted in column 8 of Table II B, application no. 17, and being depicted in the same respective line as said B1214, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “b1214-protein”, preferably it is the molecule of section (a) or (b) of this paragraph.

In particular, it was observed that in plants, especially in Arabidopsis thaliana, increasing or generating the activity of a gene product non-targeted with the activity of a “b1214-protein”, preferably being encoded by a gene comprising the nucleic acid sequence SEQ ID NO. 144857, preferably the coding region thereof, conferred the production of or the increase in glucose-1-phosphate compared with the wild type control.

The nucleic acid sequence of B1214 from Escherichia coli, e.g. as shown in the respective line in column 5 of Table I, application no. 17, has been published in B. And the activity of the gene product thereof is the activity of b1214-protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “b1214-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B1214, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1214, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1214, or a functional equivalent or a homolog thereof as depicted in column 8 of Table II, application no. 17, preferably a homolog or functional equivalent as depicted in column 8 of Table II B, application no. 17, and being depicted in the same respective line as said B1214, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “b1214-protein”, preferably it is the molecule of section (a) or (b) of this paragraph.

In particular, it was observed that in plants, especially in Arabidopsis thaliana, increasing or generating the activity of a gene product non-targeted with the activity of a “b1214-protein”, preferably being encoded by a gene comprising the nucleic acid sequence SEQ ID NO. 144857, preferably the coding region thereof, conferred the production of or the increase in 3,4,5-trihydroxypentanoic acid compared with the wild type control.

The nucleic acid sequence of B1219 from Escherichia coli, e.g. as shown in the respective line in column 5 of Table I, application no. 17, has been published in B. And the activity of the gene product thereof is the activity of b1219-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “b1219-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B1219, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1219, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1219, or a functional equivalent or a homolog thereof as depicted in column 8 of Table II, application no. 17, preferably a homolog or functional equivalent as depicted in column 8 of Table II B, application no. 17, and being depicted in the same respective line as said B1219, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “b1219-protein”, preferably it is the molecule of section (a) or (b) of this paragraph.

In particular, it was observed that in plants, especially in Arabidopsis thaliana, increasing or generating the activity of a gene product non-targeted with the activity of a “b1219-protein”, preferably being encoded by a gene comprising the nucleic acid sequence SEQ ID NO. 112072, preferably the coding region thereof, conferred the production of or the increase in glucose-1-phosphate compared with the wild type control.

The nucleic acid sequence of B1219 from Escherichia coli, e.g. as shown in the respective line in column 5 of Table I, application no. 17, has been published in B. And the activity of the gene product thereof is the activity of b1219-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “b1219-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B1219, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1219, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1219, or a functional equivalent or a homolog thereof as depicted in column 8 of Table II, application no. 17, preferably a homolog or functional equivalent as depicted in column 8 of Table II B, application no. 17, and being depicted in the same respective line as said B1219, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “b1219-protein”, preferably it is the molecule of section (a) or (b) of this paragraph.

In particular, it was observed that in plants, especially in Arabidopsis thaliana, increasing or generating the activity of a gene product non-targeted with the activity of a “b1219-protein”, preferably being encoded by a gene comprising the nucleic acid sequence SEQ ID NO. 112072, preferably the coding region thereof, conferred the production of or the increase in myo-inositol compared with the wild type control.

The nucleic acid sequence of B1219 from Escherichia coli, e.g. as shown in the respective line in column 5 of Table I, application no. 17, has been published in B. And the activity of the gene product thereof is the activity of b1219-protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “b1219-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B1219, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1219, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1219, or a functional equivalent or a homolog thereof as depicted in column 8 of Table II, application no. 17, preferably a homolog or functional equivalent as depicted in column 8 of Table II B, application no. 17, and being depicted in the same respective line as said B1219, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “b1219-protein”, preferably it is the molecule of section (a) or (b) of this paragraph.

In particular, it was observed that in plants, especially in Arabidopsis thaliana, increasing or generating the activity of a gene product non-targeted with the activity of a “b1219-protein”, preferably being encoded by a gene comprising the nucleic acid sequence SEQ ID NO. 112072, preferably the coding region thereof, conferred the production of or the increase in 3,4,5-trihydroxypentanoic acid compared with the wild type control.

The nucleic acid sequence of B1234 from Escherichia coli, e.g. as shown in the respective line in column 5 of Table I, application no. 17, has been published in B. And the activity of the gene product thereof is the activity of b1234-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “b1234-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B1234, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1234, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1234, or a functional equivalent or a homolog thereof as depicted in column 8 of Table II, application no. 17, preferably a homolog or functional equivalent as depicted in column 8 of Table II B, application no. 17, and being depicted in the same respective line as said B1234, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “b1234-protein”, preferably it is the molecule of section (a) or (b) of this paragraph.

In particular, it was observed that in plants, especially in Arabidopsis thaliana, increasing or generating the activity of a gene product non-targeted with the activity of a “b1234-protein”, preferably being encoded by a gene comprising the nucleic acid sequence SEQ ID NO. 112111, preferably the coding region thereof, conferred the production of or the increase in myo-inositol compared with the wild type control.

The nucleic acid sequence of B1249 from Escherichia coli, e.g. as shown in the respective line in column 5 of Table I, application no. 17, has been published in B. And the activity of the gene product thereof is the activity of cardiolipin synthase.

Accordingly, in one embodiment, the process of the present invention for producing galactinol in a non-human organism, like a microorganism or a plant or a part thereof, comprises increasing or generating the activity of a gene product with the activity of a gene product conferring the activity of “cardiolipin synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical galactinol), application no. 17, preferably the coding region thereof, or a homolog or a fragment thereof, and being depicted in the same respective line as said B1249, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1249, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1249, or a functional equivalent or a homolog thereof as depicted in column 8 of Table II, application no. 17, preferably a homolog or functional equivalent as depicted in column 8 of Table II B, application no. 17, and being depicted in the same respective line as said B1249, and preferably the activity is increased plastidic, whereby the respective line disclose in column 7 the fine chemical galactinol.

Accordingly, in one embodiment, the molecule which activity is to be increased in the process of the invention is the gene product with an activity as a “cardiolipin synthase”, preferably it is the molecule of section (a) or (b) of this paragraph.

In particular, it was observed that in plants, especially in Arabidopsis thaliana, increasing or generating the activity of a gene product plastidic with the activity of a “cardiolipin synthase”, preferably being encoded by a gene comprising the nucleic acid sequence SEQ ID NO. 98778, preferably the coding region thereof, conferred the production of or the increase in galactinol compared with the wild type control.

The nucleic acid sequence of B1249 from Escherichia coli, e.g. as shown in the respective line in column 5 of Table I, application no. 17, has been published in B. And the activity of the gene product thereof is the activity of cardiolipin synthase.

Accordingly, in one embodiment, the process of the present invention for producing galactitol in a non-human organism, like a microorganism or a plant or a part thereof, comprises increasing or generating the activity of a gene product with the activity of a gene product conferring the activity of “cardiolipin synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical galactitol), application no. 17, preferably the coding region thereof, or a homolog or a fragment thereof, and being depicted in the same respective line as said B1249, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1249, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1249, or a functional equivalent or a homolog thereof as depicted in column 8 of Table II, application no. 17, preferably a homolog or functional equivalent as depicted in column 8 of Table II B, application no. 17, and being depicted in the same respective line as said B1249, and preferably the activity is increased plastidic, whereby the respective line disclose in column 7 the fine chemical galactitol.

Accordingly, in one embodiment, the molecule which activity is to be increased in the process of the invention is the gene product with an activity as a “cardiolipin synthase”, preferably it is the molecule of section (a) or (b) of this paragraph.

In particular, it was observed that in plants, especially in Arabidopsis thaliana, increasing or generating the activity of a gene product plastidic with the activity of a “cardiolipin synthase”, preferably being encoded by a gene comprising the nucleic acid sequence SEQ ID NO. 98778, preferably the coding region thereof, conferred the production of or the increase in galactitol compared with the wild type control.

The nucleic acid sequence of B1249 from Escherichia coli, e.g. as shown in the respective line in column 5 of Table I, application no. 17, has been published in B. And the activity of the gene product thereof is the activity of cardiolipin synthase.

Accordingly, in one embodiment, the process of the present invention for producing sorbitol in a non-human organism, like a microorganism or a plant or a part thereof, comprises increasing or generating the activity of a gene product with the activity of a gene product conferring the activity of “cardiolipin synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical sorbitol), application no. 17, preferably the coding region thereof, or a homolog or a fragment thereof, and being depicted in the same respective line as said B1249, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1249, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1249, or a functional equivalent or a homolog thereof as depicted in column 8 of Table II, application no. 17, preferably a homolog or functional equivalent as depicted in column 8 of Table II B, application no. 17, and being depicted in the same respective line as said B1249, and preferably the activity is increased plastidic, whereby the respective line disclose in column 7 the fine chemical sorbitol.

Accordingly, in one embodiment, the molecule which activity is to be increased in the process of the invention is the gene product with an activity as a “cardiolipin synthase”, preferably it is the molecule of section (a) or (b) of this paragraph.

In particular, it was observed that in plants, especially in Arabidopsis thaliana, increasing or generating the activity of a gene product plastidic with the activity of a “cardiolipin synthase”, preferably being encoded by a gene comprising the nucleic acid sequence SEQ ID NO. 98778, preferably the coding region thereof, conferred the production of or the increase in sorbitol compared with the wild type control.

The nucleic acid sequence of B1254 from Escherichia coli, e.g. as shown in the respective line in column 5 of Table I, application no. 17, has been published in B. And the activity of the gene product thereof is the activity of intracellular septation protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “intracellular septation protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B1254, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1254, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1254, or a functional equivalent or a homolog thereof as depicted in column 8 of Table II, application no. 17, preferably a homolog or functional equivalent as depicted in column 8 of Table II B, application no. 17, and being depicted in the same respective line as said B1254, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “intracellular septation protein”, preferably it is the molecule of section (a) or (b) of this paragraph.

In particular, it was observed that in plants, especially in Arabidopsis thaliana, increasing or generating the activity of a gene product non-targeted with the activity of a “intracellular septation protein”, preferably being encoded by a gene comprising the nucleic acid sequence SEQ ID NO. 144894, preferably the coding region thereof, conferred the production of or the increase in 3,4,5-trihydroxypentanoic acid compared with the wild type control.

The nucleic acid sequence of B1255 from Escherichia coli, e.g. as shown in the respective line in column 5 of Table I, application no. 17, has been published in B. And the activity of the gene product thereof is the activity of membrane protein.

Accordingly, in one embodiment, the process of the present invention for producing fructose in a non-human organism, like a microorganism or a plant or a part thereof, comprises increasing or generating the activity of a gene product with the activity of a gene product conferring the activity of “membrane protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B1255, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1255, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1255, or a functional equivalent or a homolog thereof as depicted in column 8 of Table II, application no. 17, preferably a homolog or functional equivalent as depicted in column 8 of Table II B, application no. 17, and being depicted in the same respective line as said B1255, and preferably the activity is increased non-targeted, whereby the respective line disclose in column 7 the fine chemical fructose.

Accordingly, in one embodiment, the molecule which activity is to be increased in the process of the invention is the gene product with an activity as a “membrane protein”, preferably it is the molecule of section (a) or (b) of this paragraph.

In particular, it was observed that in plants, especially in Arabidopsis thaliana, increasing or generating the activity of a gene product non-targeted with the activity of a “membrane protein”, preferably being encoded by a gene comprising the nucleic acid sequence SEQ ID NO. 37483, preferably the coding region thereof, conferred the production of or the increase in fructose compared with the wild type control.

The nucleic acid sequence of B1255 from Escherichia coli, e.g. as shown in the respective line in column 5 of Table I, application no. 17, has been published in B. And the activity of the gene product thereof is the activity of membrane protein.

Accordingly, in one embodiment, the process of the present invention for producing glucose in a non-human organism, like a microorganism or a plant or a part thereof, comprises increasing or generating the activity of a gene product with the activity of a gene product conferring the activity of “membrane protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B1255, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1255, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1255, or a functional equivalent or a homolog thereof as depicted in column 8 of Table II, application no. 17, preferably a homolog or functional equivalent as depicted in column 8 of Table II B, application no. 17, and being depicted in the same respective line as said B1255, and preferably the activity is increased non-targeted, whereby the respective line disclose in column 7 the fine chemical glucose.

Accordingly, in one embodiment, the molecule which activity is to be increased in the process of the invention is the gene product with an activity as a “membrane protein”, preferably it is the molecule of section (a) or (b) of this paragraph.

In particular, it was observed that in plants, especially in Arabidopsis thaliana, increasing or generating the activity of a gene product non-targeted with the activity of a “membrane protein”, preferably being encoded by a gene comprising the nucleic acid sequence SEQ ID NO. 37483, preferably the coding region thereof, conferred the production of or the increase in glucose compared with the wild type control.

The nucleic acid sequence of B1255 from Escherichia coli, e.g. as shown in the respective line in column 5 of Table I, application no. 17, has been published in B. And the activity of the gene product thereof is the activity of membrane protein.

Accordingly, in one embodiment, the process of the present invention for producing raffinose in a non-human organism, like a microorganism or a plant or a part thereof, comprises increasing or generating the activity of a gene product with the activity of a gene product conferring the activity of “membrane protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said B1255, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1255, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1255, or a functional equivalent or a homolog thereof as depicted in column 8 of Table II, application no. 17, preferably a homolog or functional equivalent as depicted in column 8 of Table II B, application no. 17, and being depicted in the same respective line as said B1255, and preferably the activity is increased non-targeted, whereby the respective line disclose in column 7 the fine chemical raffinose.

Accordingly, in one embodiment, the molecule which activity is to be increased in the process of the invention is the gene product with an activity as a “membrane protein”, preferably it is the molecule of section (a) or (b) of this paragraph.

In particular, it was observed that in plants, especially in Arabidopsis thaliana, increasing or generating the activity of a gene product non-targeted with the activity of a “membrane protein”, preferably being encoded by a gene comprising the nucleic acid sequence SEQ ID NO. 37483, preferably the coding region thereof, conferred the production of or the increase in raffinose compared with the wild type control.

The nucleic acid sequence of B1255 from Escherichia coli, e.g. as shown in the respective line in column 5 of Table I, application no. 17, has been published in B. And the activity of the gene product thereof is the activity of membrane protein.

Accordingly, in one embodiment, the process of the present invention for producing sucrose in a non-human organism, like a microorganism or a plant or a part thereof, comprises increasing or generating the activity of a gene product with the activity of a gene product conferring the activity of “membrane protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said B1255, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1255, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1255, or a functional equivalent or a homolog thereof as depicted in column 8 of Table II, application no. 17, preferably a homolog or functional equivalent as depicted in column 8 of Table II B, application no. 17, and being depicted in the same respective line as said B1255, and preferably the activity is increased non-targeted, whereby the respective line disclose in column 7 the fine chemical sucrose.

Accordingly, in one embodiment, the molecule which activity is to be increased in the process of the invention is the gene product with an activity as a “membrane protein”, preferably it is the molecule of section (a) or (b) of this paragraph.

In particular, it was observed that in plants, especially in Arabidopsis thaliana, increasing or generating the activity of a gene product non-targeted with the activity of a “membrane protein”, preferably being encoded by a gene comprising the nucleic acid sequence SEQ ID NO. 37483, preferably the coding region thereof, conferred the production of or the increase in sucrose compared with the wild type control.

The nucleic acid sequence of B1262 from Escherichia coli, e.g. as shown in the respective line in column 5 of Table I, application no. 17, has been published in B. And the activity of the gene product thereof is the activity of tryptophan biosynthesis protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “tryptophan biosynthesis protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B1262, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1262, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1262, or a functional equivalent or a homolog thereof as depicted in column 8 of Table II, application no. 17, preferably a homolog or functional equivalent as depicted in column 8 of Table II B, application no. 17, and being depicted in the same respective line as said B1262, and preferably the activity is increased plastidic, whereby the respective line disclose in column 7 the fine chemical myo-inositol.

Accordingly, in one embodiment, the molecule which activity is to be increased in the process of the invention is the gene product with an activity as a “tryptophan biosynthesis protein”, preferably it is the molecule of section (a) or (b) of this paragraph.

In particular, it was observed that in plants, especially in Arabidopsis thaliana, increasing or generating the activity of a gene product plastidic with the activity of a “tryptophan biosynthesis protein”, preferably being encoded by a gene comprising the nucleic acid sequence SEQ ID NO. 76157, preferably the coding region thereof, conferred the production of or the increase in myo-inositol compared with the wild type control.

The nucleic acid sequence of B1285 from Escherichia coli, e.g. as shown in the respective line in column 5 of Table I, application no. 17, has been published in B. And the activity of the gene product thereof is the activity of b1285-protein.

Accordingly, in one embodiment, the process of the present invention for producing fructose in a non-human organism, like a microorganism or a plant or a part thereof, comprises increasing or generating the activity of a gene product with the activity of a gene product conferring the activity of “b1285-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B1285, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1285, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1285, or a functional equivalent or a homolog thereof as depicted in column 8 of Table II, application no. 17, preferably a homolog or functional equivalent as depicted in column 8 of Table II B, application no. 17, and being depicted in the same respective line as said B1285, and preferably the activity is increased non-targeted, whereby the respective line disclose in column 7 the fine chemical fructose.

Accordingly, in one embodiment, the molecule which activity is to be increased in the process of the invention is the gene product with an activity as a “b1285-protein”, preferably it is the molecule of section (a) or (b) of this paragraph.

In particular, it was observed that in plants, especially in Arabidopsis thaliana, increasing or generating the activity of a gene product non-targeted with the activity of a “b1285-protein”, preferably being encoded by a gene comprising the nucleic acid sequence SEQ ID NO. 134655, preferably the coding region thereof, conferred the production of or the increase in fructose compared with the wild type control.

The nucleic acid sequence of B1356 from Escherichia coli, e.g. as shown in the respective line in column 5 of Table I, application no. 17, has been published in B. And the activity of the gene product thereof is the activity of phage-related repressor protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “phage-related repressor protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B1356, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1356, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1356, or a functional equivalent or a homolog thereof as depicted in column 8 of Table II, application no. 17, preferably a homolog or functional equivalent as depicted in column 8 of Table II B, application no. 17, and being depicted in the same respective line as said B1356, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “phage-related repressor protein”, preferably it is the molecule of section (a) or (b) of this paragraph.

In particular, it was observed that in plants, especially in Arabidopsis thaliana, increasing or generating the activity of a gene product non-targeted with the activity of a “phage-related repressor protein”, preferably being encoded by a gene comprising the nucleic acid sequence SEQ ID NO. 124557, preferably the coding region thereof, conferred the production of or the increase in glucose-1-phosphate compared with the wild type control.

The nucleic acid sequence of B1357 from Escherichia coli, e.g. as shown in the respective line in column 5 of Table I, application no. 17, has been published in B. And the activity of the gene product thereof is the activity of b1357-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “b1357-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B1357, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1357, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1357, or a functional equivalent or a homolog thereof as depicted in column 8 of Table II, application no. 17, preferably a homolog or functional equivalent as depicted in column 8 of Table II B, application no. 17, and being depicted in the same respective line as said B1357, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “b1357-protein”, preferably it is the molecule of section (a) or (b) of this paragraph.

In particular, it was observed that in plants, especially in Arabidopsis thaliana, increasing or generating the activity of a gene product non-targeted with the activity of a “b1357-protein”, preferably being encoded by a gene comprising the nucleic acid sequence SEQ ID NO. 145066, preferably the coding region thereof, conferred the production of or the increase in glucose-1-phosphate compared with the wild type control.

The nucleic acid sequence of B1390 from Escherichia coli, e.g. as shown in the respective line in column 5 of Table I, application no. 17, has been published in B. And the activity of the gene product thereof is the activity of phenylacetic acid degradation protein.

Accordingly, in one embodiment, the process of the present invention for producing glucose in a non-human organism, like a microorganism or a plant or a part thereof, comprises increasing or generating the activity of a gene product with the activity of a gene product conferring the activity of “phenylacetic acid degradation protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B1390, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1390, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1390, or a functional equivalent or a homolog thereof as depicted in column 8 of Table II, application no. 17, preferably a homolog or functional equivalent as depicted in column 8 of Table II B, application no. 17, and being depicted in the same respective line as said B1390, and preferably the activity is increased non-targeted, whereby the respective line disclose in column 7 the fine chemical glucose.

Accordingly, in one embodiment, the molecule which activity is to be increased in the process of the invention is the gene product with an activity as a “phenylacetic acid degradation protein”, preferably it is the molecule of section (a) or (b) of this paragraph.

In particular, it was observed that in plants, especially in Arabidopsis thaliana, increasing or generating the activity of a gene product non-targeted with the activity of a “phenylacetic acid degradation protein”, preferably being encoded by a gene comprising the nucleic acid sequence SEQ ID NO. 134697, preferably the coding region thereof, conferred the production of or the increase in glucose compared with the wild type control.

The nucleic acid sequence of B1391 from Escherichia coli, e.g. as shown in the respective line in column 5 of Table I, application no. 17, has been published in B. And the activity of the gene product thereof is the activity of phenylacetic acid degradation protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “phenylacetic acid degradation protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B1391, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1391, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1391, or a functional equivalent or a homolog thereof as depicted in column 8 of Table II, application no. 17, preferably a homolog or functional equivalent as depicted in column 8 of Table II B, application no. 17, and being depicted in the same respective line as said B1391, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “phenylacetic acid degradation protein”, preferably it is the molecule of section (a) or (b) of this paragraph.

In particular, it was observed that in plants, especially in Arabidopsis thaliana, increasing or generating the activity of a gene product non-targeted with the activity of a “phenylacetic acid degradation protein”, preferably being encoded by a gene comprising the nucleic acid sequence SEQ ID NO. 131489, preferably the coding region thereof, conferred the production of or the increase in 3,4,5-trihydroxypentanoic acid compared with the wild type control.

The nucleic acid sequence of B1410 from Escherichia coli, e.g. as shown in the respective line in column 5 of Table I, application no. 17, has been published in B. And the activity of the gene product thereof is the activity of b1410-protein.

Accordingly, in one embodiment, the process of the present invention for producing sedoheptulose-7-phosphate in a non-human organism, like a microorganism or a plant or a part thereof, comprises increasing or generating the activity of a gene product with the activity of a gene product conferring the activity of “b1410-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical sedoheptulose-7-phosphate), application no. 17, preferably the coding region thereof, or a homolog or a fragment thereof, and being depicted in the same respective line as said B1410, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1410, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1410, or a functional equivalent or a homolog thereof as depicted in column 8 of Table II, application no. 17, preferably a homolog or functional equivalent as depicted in column 8 of Table II B, application no. 17, and being depicted in the same respective line as said B1410, and preferably the activity is increased plastidic, whereby the respective line disclose in column 7 the fine chemical sedoheptulose-7-phosphate.

Accordingly, in one embodiment, the molecule which activity is to be increased in the process of the invention is the gene product with an activity as a “b1410-protein”, preferably it is the molecule of section (a) or (b) of this paragraph.

In particular, it was observed that in plants, especially in Arabidopsis thaliana, increasing or generating the activity of a gene product plastidic with the activity of a “b1410-protein”, preferably being encoded by a gene comprising the nucleic acid sequence SEQ ID NO. 145091, preferably the coding region thereof, conferred the production of or the increase in sedoheptulose7-phosphate compared with the wild type control.

The nucleic acid sequence of B1425 from Escherichia coli, e.g. as shown in the respective line in column 5 of Table I, application no. 17, has been published in B. And the activity of the gene product thereof is the activity of b1425-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “b1425-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B1425, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1425, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1425, or a functional equivalent or a homolog thereof as depicted in column 8 of Table II, application no. 17, preferably a homolog or functional equivalent as depicted in column 8 of Table II B, application no. 17, and being depicted in the same respective line as said B1425, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “b1425-protein”, preferably it is the molecule of section (a) or (b) of this paragraph.

In particular, it was observed that in plants, especially in Arabidopsis thaliana, increasing or generating the activity of a gene product non-targeted with the activity of a “b1425-protein”, preferably being encoded by a gene comprising the nucleic acid sequence SEQ ID NO. 134756, preferably the coding region thereof, conferred the production of or the increase in myo-inositol compared with the wild type control.

The nucleic acid sequence of B1431 from Escherichia coli, e.g. as shown in the respective line in column 5 of Table I, application no. 17, has been published in B. And the activity of the gene product thereof is the activity of lipoprotein precursor.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “lipoprotein precursor”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B1431, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1431, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1431, or a functional equivalent or a homolog thereof as depicted in column 8 of Table II, application no. 17, preferably a homolog or functional equivalent as depicted in column 8 of Table II B, application no. 17, and being depicted in the same respective line as said B1431, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “lipoprotein precursor”, preferably it is the molecule of section (a) or (b) of this paragraph.

In particular, it was observed that in plants, especially in Arabidopsis thaliana, increasing or generating the activity of a gene product plastidic with the activity of a “lipoprotein precursor”, preferably being encoded by a gene comprising the nucleic acid sequence SEQ ID NO. 38266, preferably the coding region thereof, conferred the production of or the increase in glucose-1-phosphate compared with the wild type control.

The nucleic acid sequence of B1431 from Escherichia coli, e.g. as shown in the respective line in column 5 of Table I, application no. 17, has been published in B. And the activity of the gene product thereof is the activity of lipoprotein precursor.

Accordingly, in one embodiment, the process of the present invention for producing sedoheptulose-7-phosphate in a non-human organism, like a microorganism or a plant or a part thereof, comprises increasing or generating the activity of a gene product with the activity of a gene product conferring the activity of “lipoprotein precursor”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical sedoheptulose-7-phosphate), application no. 17, preferably the coding region thereof, or a homolog or a fragment thereof, and being depicted in the same respective line as said B1431, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1431, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1431, or a functional equivalent or a homolog thereof as depicted in column 8 of Table II, application no. 17, preferably a homolog or functional equivalent as depicted in column 8 of Table II B, application no. 17, and being depicted in the same respective line as said B1431, and preferably the activity is increased plastidic, whereby the respective line disclose in column 7 the fine chemical sedoheptulose-7-phosphate.

Accordingly, in one embodiment, the molecule which activity is to be increased in the process of the invention is the gene product with an activity as a “lipoprotein precursor”, preferably it is the molecule of section (a) or (b) of this paragraph.

In particular, it was observed that in plants, especially in Arabidopsis thaliana, increasing or generating the activity of a gene product plastidic with the activity of a “lipoprotein precursor”, preferably being encoded by a gene comprising the nucleic acid sequence SEQ ID NO. 38266, preferably the coding region thereof, conferred the production of or the increase in sedoheptulose-7-phosphate compared with the wild type control.

The nucleic acid sequence of B1431 from Escherichia coli, e.g. as shown in the respective line in column 5 of Table I, application no. 17, has been published in B. And the activity of the gene product thereof is the activity of lipoprotein precursor.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “lipoprotein precursor”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B1431, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1431, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1431, or a functional equivalent or a homolog thereof as depicted in column 8 of Table II, application no. 17, preferably a homolog or functional equivalent as depicted in column 8 of Table II B, application no. 17, and being depicted in the same respective line as said B1431, and preferably the activity is increased plastidic, whereby the respective line disclose in column 7 the fine chemical 3,4,5-trihydroxypentanoic acid.

Accordingly, in one embodiment, the molecule which activity is to be increased in the process of the invention is the gene product with an activity as a “lipoprotein precursor”, preferably it is the molecule of section (a) or (b) of this paragraph.

In particular, it was observed that in plants, especially in Arabidopsis thaliana, increasing or generating the activity of a gene product plastidic with the activity of a “lipoprotein precursor”, preferably being encoded by a gene comprising the nucleic acid sequence SEQ ID NO. 38266, preferably the coding region thereof, conferred the production of or the increase in 3,4,5-trihydroxypentanoic acid compared with the wild type control.

The nucleic acid sequence of B1445 from Escherichia coli, e.g. as shown in the respective line in column 5 of Table I, application no. 17, has been published in B. And the activity of the gene product thereof is the activity of b1445-protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “b1445-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B1445, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1445, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1445, or a functional equivalent or a homolog thereof as depicted in column 8 of Table II, application no. 17, preferably a homolog or functional equivalent as depicted in column 8 of Table II B, application no. 17, and being depicted in the same respective line as said B1445, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “b1445-protein”, preferably it is the molecule of section (a) or (b) of this paragraph.

In particular, it was observed that in plants, especially in Arabidopsis thaliana, increasing or generating the activity of a gene product non-targeted with the activity of a “b1445-protein”, preferably being encoded by a gene comprising the nucleic acid sequence SEQ ID NO. 38289, preferably the coding region thereof, conferred the production of or the increase in 3,4,5-trihydroxypentanoic acid compared with the wild type control.

The nucleic acid sequence of B1470 from Escherichia coli, e.g. as shown in the respective line in column 5 of Table I, application no. 17, has been published in B. And the activity of the gene product thereof is the activity of b1470-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “b1470-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B1470, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1470, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1470, or a functional equivalent or a homolog thereof as depicted in column 8 of Table II, application no. 17, preferably a homolog or functional equivalent as depicted in column 8 of Table II B, application no. 17, and being depicted in the same respective line as said B1470, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “b1470-protein”, preferably it is the molecule of section (a) or (b) of this paragraph.

In particular, it was observed that in plants, especially in Arabidopsis thaliana, increasing or generating the activity of a gene product non-targeted with the activity of a “b1470-protein”, preferably being encoded by a gene comprising the nucleic acid sequence SEQ ID NO. 115056, preferably the coding region thereof, conferred the production of or the increase in glucose-1-phosphate compared with the wild type control.

The nucleic acid sequence of B1522 from Escherichia coli, e.g. as shown in the respective line in column 5 of Table I, application no. 17, has been published in B. And the activity of the gene product thereof is the activity of b1522-protein.

Accordingly, in one embodiment, the process of the present invention for producing fructose in a non-human organism, like a microorganism or a plant or a part thereof, comprises increasing or generating the activity of a gene product with the activity of a gene product conferring the activity of “b1522-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B1522, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1522, or a functional equivalent or a homolog thereof as depicted in column 8 of Table II, application no. 17, preferably a homolog or functional equivalent as depicted in column 8 of Table II B, application no. 17, and being depicted in the same respective line as said B1522, and preferably the activity is increased non-targeted, whereby the respective line disclose in column 7 the fine chemical fructose.

Accordingly, in one embodiment, the molecule which activity is to be increased in the process of the invention is the gene product with an activity as a “b1522-protein”, preferably it is the molecule of section (a) or (b) of this paragraph.

In particular, it was observed that in plants, especially in Arabidopsis thaliana, increasing or generating the activity of a gene product non-targeted with the activity of a “b1522-protein”, preferably being encoded by a gene comprising the nucleic acid sequence SEQ ID NO. 7947, preferably the coding region thereof, conferred the production of or the increase in fructose compared with the wild type control.

The nucleic acid sequence of B1522 from Escherichia coli, e.g. as shown in the respective line in column 5 of Table I, application no. 17, has been published in B. And the activity of the gene product thereof is the activity of b1522-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “b1522-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B1522, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1522, or a functional equivalent or a homolog thereof as depicted in column 8 of Table II, application no. 17, preferably a homolog or functional equivalent as depicted in column 8 of Table II B, application no. 17, and being depicted in the same respective line as said B1522, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “b1522-protein”, preferably it is the molecule of section (a) or (b) of this paragraph.

In particular, it was observed that in plants, especially in Arabidopsis thaliana, increasing or generating the activity of a gene product non-targeted with the activity of a “b1522-protein”, preferably being encoded by a gene comprising the nucleic acid sequence SEQ ID NO. 7947, preferably the coding region thereof, conferred the production of or the increase in glucose-1-phosphate compared with the wild type control.

The nucleic acid sequence of B1556 from Escherichia coli, e.g. as shown in the respective line in column 5 of Table I, application no. 17, has been published in B. And the activity of the gene product thereof is the activity of b1556-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “b1556-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B1556, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1556, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1556, or a functional equivalent or a homolog thereof as depicted in column 8 of Table II, application no. 17, preferably a homolog or functional equivalent as depicted in column 8 of Table II B, application no. 17, and being depicted in the same respective line as said B1556, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “b1556-protein”, preferably it is the molecule of section (a) or (b) of this paragraph.

In particular, it was observed that in plants, especially in Arabidopsis thaliana, increasing or generating the activity of a gene product plastidic with the activity of a “b1556-protein”, preferably being encoded by a gene comprising the nucleic acid sequence SEQ ID NO. 145142, preferably the coding region thereof, conferred the production of or the increase in glucose-1-phosphate compared with the wild type control.

The nucleic acid sequence of B1556 from Escherichia coli, e.g. as shown in the respective line in column 5 of Table I, application no. 17, has been published in B. And the activity of the gene product thereof is the activity of b1556-protein.

Accordingly, in one embodiment, the process of the present invention for producing sedoheptulose-7-phosphate in a non-human organism, like a microorganism or a plant or a part thereof, comprises increasing or generating the activity of a gene product with the activity of a gene product conferring the activity of “b1556-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical sedoheptulose-7-phosphate), application no. 17, preferably the coding region thereof, or a homolog or a fragment thereof, and being depicted in the same respective line as said B1556, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1556, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1556, or a functional equivalent or a homolog thereof as depicted in column 8 of Table II, application no. 17, preferably a homolog or functional equivalent as depicted in column 8 of Table II B, application no. 17, and being depicted in the same respective line as said B1556, and preferably the activity is increased plastidic, whereby the respective line disclose in column 7 the fine chemical sedoheptulose-7-phosphate.

Accordingly, in one embodiment, the molecule which activity is to be increased in the process of the invention is the gene product with an activity as a “b1556-protein”, preferably it is the molecule of section (a) or (b) of this paragraph.

In particular, it was observed that in plants, especially in Arabidopsis thaliana, increasing or generating the activity of a gene product plastidic with the activity of a “b1556-protein”, preferably being encoded by a gene comprising the nucleic acid sequence SEQ ID NO. 145142, preferably the coding region thereof, conferred the production of or the increase in sedoheptulose7-phosphate compared with the wild type control.

The nucleic acid sequence of B1583 from Escherichia coli, e.g. as shown in the respective line in column 5 of Table I, application no. 17, has been published in B. And the activity of the gene product thereof is the activity of b1583-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “b1583-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B1583, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1583, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1583, or a functional equivalent or a homolog thereof as depicted in column 8 of Table II, application no. 17, preferably a homolog or functional equivalent as depicted in column 8 of Table II B, application no. 17, and being depicted in the same respective line as said B1583, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “b1583-protein”, preferably it is the molecule of section (a) or (b) of this paragraph.

In particular, it was observed that in plants, especially in Arabidopsis thaliana, increasing or generating the activity of a gene product non-targeted with the activity of a “b1583-protein”, preferably being encoded by a gene comprising the nucleic acid sequence SEQ ID NO. 78138, preferably the coding region thereof, conferred the production of or the increase in glucose-1-phosphate compared with the wild type control.

The nucleic acid sequence of B1589 from Escherichia coli, e.g. as shown in the respective line in column 5 of Table I, application no. 17, has been published in B. And the activity of the gene product thereof is the activity of Fe—S subunit of oxidoreductase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “Fe—S subunit of oxidoreductase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B1589, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1589, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1589, or a functional equivalent or a homolog thereof as depicted in column 8 of Table II, application no. 17, preferably a homolog or functional equivalent as depicted in column 8 of Table II B, application no. 17, and being depicted in the same respective line as said B1589, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “Fe—S subunit of oxidoreductase”, preferably it is the molecule of section (a) or (b) of this paragraph.

In particular, it was observed that in plants, especially in Arabidopsis thaliana, increasing or generating the activity of a gene product non-targeted with the activity of a “Fe—S subunit of oxidoreductase”, preferably being encoded by a gene comprising the nucleic acid sequence SEQ ID NO. 78154, preferably the coding region thereof, conferred the production of or the increase in glucose-1-phosphate compared with the wild type control.

The nucleic acid sequence of B1589 from Escherichia coli, e.g. as shown in the respective line in column 5 of Table I, application no. 17, has been published in B. And the activity of the gene product thereof is the activity of Fe—S subunit of oxidoreductase.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “Fe—S subunit of oxidoreductase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B1589, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1589, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1589, or a functional equivalent or a homolog thereof as depicted in column 8 of Table II, application no. 17, preferably a homolog or functional equivalent as depicted in column 8 of Table II B, application no. 17, and being depicted in the same respective line as said B1589, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “Fe—S subunit of oxidoreductase”, preferably it is the molecule of section (a) or (b) of this paragraph.

In particular, it was observed that in plants, especially in Arabidopsis thaliana, increasing or generating the activity of a gene product non-targeted with the activity of a “Fe—S subunit of oxidoreductase”, preferably being encoded by a gene comprising the nucleic acid sequence SEQ ID NO. 78154, preferably the coding region thereof, conferred the production of or the increase in 3,4,5-trihydroxypentanoic acid compared with the wild type control.

The nucleic acid sequence of B1597 from Escherichia coli, e.g. as shown in the respective line in column 5 of Table I, application no. 17, has been published in B. And the activity of the gene product thereof is the activity of acid shock protein.

Accordingly, in one embodiment, the process of the present invention for producing fructose in a non-human organism, like a microorganism or a plant or a part thereof, comprises increasing or generating the activity of a gene product with the activity of a gene product conferring the activity of “acid shock protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B1597, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1597, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1597, or a functional equivalent or a homolog thereof as depicted in column 8 of Table II, application no. 17, preferably a homolog or functional equivalent as depicted in column 8 of Table II B, application no. 17, and being depicted in the same respective line as said B1597, and preferably the activity is increased non-targeted, whereby the respective line disclose in column 7 the fine chemical fructose.

Accordingly, in one embodiment, the molecule which activity is to be increased in the process of the invention is the gene product with an activity as a “acid shock protein”, preferably it is the molecule of section (a) or (b) of this paragraph.

In particular, it was observed that in plants, especially in Arabidopsis thaliana, increasing or generating the activity of a gene product non-targeted with the activity of a “acid shock protein”, preferably being encoded by a gene comprising the nucleic acid sequence SEQ ID NO. 38300, preferably the coding region thereof, conferred the production of or the increase in fructose compared with the wild type control.

The nucleic acid sequence of B1597 from Escherichia coli, e.g. as shown in the respective line in column 5 of Table I, application no. 17, has been published in B. And the activity of the gene product thereof is the activity of acid shock protein.

Accordingly, in one embodiment, the process of the present invention for producing glucose in a non-human organism, like a microorganism or a plant or a part thereof, comprises increasing or generating the activity of a gene product with the activity of a gene product conferring the activity of “acid shock protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B1597, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1597, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1597, or a functional equivalent or a homolog thereof as depicted in column 8 of Table II, application no. 17, preferably a homolog or functional equivalent as depicted in column 8 of Table II B, application no. 17, and being depicted in the same respective line as said B1597, and preferably the activity is increased non-targeted, whereby the respective line disclose in column 7 the fine chemical glucose.

Accordingly, in one embodiment, the molecule which activity is to be increased in the process of the invention is the gene product with an activity as a “acid shock protein”, preferably it is the molecule of section (a) or (b) of this paragraph.

In particular, it was observed that in plants, especially in Arabidopsis thaliana, increasing or generating the activity of a gene product non-targeted with the activity of a “acid shock protein”, preferably being encoded by a gene comprising the nucleic acid sequence SEQ ID NO. 38300, preferably the coding region thereof, conferred the production of or the increase in glucose compared with the wild type control.

The nucleic acid sequence of B1597 from Escherichia coli, e.g. as shown in the respective line in column 5 of Table I, application no. 17, has been published in B. And the activity of the gene product thereof is the activity of acid shock protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “acid shock protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B1597, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1597, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1597, or a functional equivalent or a homolog thereof as depicted in column 8 of Table II, application no. 17, preferably a homolog or functional equivalent as depicted in column 8 of Table II B, application no. 17, and being depicted in the same respective line as said B1597, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “acid shock protein”, preferably it is the molecule of section (a) or (b) of this paragraph.

In particular, it was observed that in plants, especially in Arabidopsis thaliana, increasing or generating the activity of a gene product non-targeted with the activity of a “acid shock protein”, preferably being encoded by a gene comprising the nucleic acid sequence SEQ ID NO. 38300, preferably the coding region thereof, conferred the production of or the increase in glucose-1-phosphate compared with the wild type control.

The nucleic acid sequence of B1597 from Escherichia coli, e.g. as shown in the respective line in column 5 of Table I, application no. 17, has been published in B. And the activity of the gene product thereof is the activity of acid shock protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “acid shock protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B1597, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1597, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1597, or a functional equivalent or a homolog thereof as depicted in column 8 of Table II, application no. 17, preferably a homolog or functional equivalent as depicted in column 8 of Table II B, application no. 17, and being depicted in the same respective line as said B1597, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “acid shock protein”, preferably it is the molecule of section (a) or (b) of this paragraph.

In particular, it was observed that in plants, especially in Arabidopsis thaliana, increasing or generating the activity of a gene product non-targeted with the activity of a “acid shock protein”, preferably being encoded by a gene comprising the nucleic acid sequence SEQ ID NO. 38300, preferably the coding region thereof, conferred the production of or the increase in myo-inositol compared with the wild type control.

The nucleic acid sequence of B1597 from Escherichia coli, e.g. as shown in the respective line in column 5 of Table I, application no. 17, has been published in B. And the activity of the gene product thereof is the activity of acid shock protein.

Accordingly, in one embodiment, the process of the present invention for producing raffinose in a non-human organism, like a microorganism or a plant or a part thereof, comprises increasing or generating the activity of a gene product with the activity of a gene product conferring the activity of “acid shock protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said B1597, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1597, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1597, or a functional equivalent or a homolog thereof as depicted in column 8 of Table II, application no. 17, preferably a homolog or functional equivalent as depicted in column 8 of Table II B, application no. 17, and being depicted in the same respective line as said B1597, and preferably the activity is increased non-targeted, whereby the respective line disclose in column 7 the fine chemical raffinose.

Accordingly, in one embodiment, the molecule which activity is to be increased in the process of the invention is the gene product with an activity as a “acid shock protein”, preferably it is the molecule of section (a) or (b) of this paragraph.

In particular, it was observed that in plants, especially in Arabidopsis thaliana, increasing or generating the activity of a gene product non-targeted with the activity of a “acid shock protein”, preferably being encoded by a gene comprising the nucleic acid sequence SEQ ID NO. 38300, preferably the coding region thereof, conferred the production of or the increase in raffinose compared with the wild type control.

The nucleic acid sequence of B1621 from Escherichia coli, e.g. as shown in the respective line in column 5 of Table I, application no. 17, has been published in B. And the activity of the gene product thereof is the activity of phosphotransferase system component.

Accordingly, in one embodiment, the process of the present invention for producing fructose in a non-human organism, like a microorganism or a plant or a part thereof, comprises increasing or generating the activity of a gene product with the activity of a gene product conferring the activity of “phosphotransferase system component”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B1621, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1621, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1621, or a functional equivalent or a homolog thereof as depicted in column 8 of Table II, application no. 17, preferably a homolog or functional equivalent as depicted in column 8 of Table II B, application no. 17, and being depicted in the same respective line as said B1621, and preferably the activity is increased non-targeted, whereby the respective line disclose in column 7 the fine chemical fructose.

Accordingly, in one embodiment, the molecule which activity is to be increased in the process of the invention is the gene product with an activity as a “phosphotransferase system component”, preferably it is the molecule of section (a) or (b) of this paragraph.

In particular, it was observed that in plants, especially in Arabidopsis thaliana, increasing or generating the activity of a gene product non-targeted with the activity of a “phosphotransferase system component”, preferably being encoded by a gene comprising the nucleic acid sequence SEQ ID NO. 124571, preferably the coding region thereof, conferred the production of or the increase in fructose compared with the wild type control.

The nucleic acid sequence of B1621 from Escherichia coli, e.g. as shown in the respective line in column 5 of Table I, application no. 17, has been published in B. And the activity of the gene product thereof is the activity of phosphotransferase system component.

Accordingly, in one embodiment, the process of the present invention for producing glucose in a non-human organism, like a microorganism or a plant or a part thereof, comprises increasing or generating the activity of a gene product with the activity of a gene product conferring the activity of “phosphotransferase system component”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B1621, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1621, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1621, or a functional equivalent or a homolog thereof as depicted in column 8 of Table II, application no. 17, preferably a homolog or functional equivalent as depicted in column 8 of Table II B, application no. 17, and being depicted in the same respective line as said B1621, and preferably the activity is increased non-targeted, whereby the respective line disclose in column 7 the fine chemical glucose.

Accordingly, in one embodiment, the molecule which activity is to be increased in the process of the invention is the gene product with an activity as a “phosphotransferase system component”, preferably it is the molecule of section (a) or (b) of this paragraph.

In particular, it was observed that in plants, especially in Arabidopsis thaliana, increasing or generating the activity of a gene product non-targeted with the activity of a “phosphotransferase system component”, preferably being encoded by a gene comprising the nucleic acid sequence SEQ ID NO. 124571, preferably the coding region thereof, conferred the production of or the increase in glucose compared with the wild type control.

The nucleic acid sequence of B1627 from Escherichia coli, e.g. as shown in the respective line in column 5 of Table I, application no. 17, has been published in B. And the activity of the gene product thereof is the activity of electron transport complex protein.

Accordingly, in one embodiment, the process of the present invention for producing glucose in a non-human organism, like a microorganism or a plant or a part thereof, comprises increasing or generating the activity of a gene product with the activity of a gene product conferring the activity of “electron transport complex protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B1627, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1627, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1627, or a functional equivalent or a homolog thereof as depicted in column 8 of Table II, application no. 17, preferably a homolog or functional equivalent as depicted in column 8 of Table II B, application no. 17, and being depicted in the same respective line as said B1627, and preferably the activity is increased non-targeted, whereby the respective line disclose in column 7 the fine chemical glucose.

Accordingly, in one embodiment, the molecule which activity is to be increased in the process of the invention is the gene product with an activity as a “electron transport complex protein”, preferably it is the molecule of section (a) or (b) of this paragraph.

In particular, it was observed that in plants, especially in Arabidopsis thaliana, increasing or generating the activity of a gene product non-targeted with the activity of a “electron transport complex protein”, preferably being encoded by a gene comprising the nucleic acid sequence SEQ ID NO. 38345, preferably the coding region thereof, conferred the production of or the increase in glucose compared with the wild type control.

The nucleic acid sequence of B1627 from Escherichia coli, e.g. as shown in the respective line in column 5 of Table I, application no. 17, has been published in B. And the activity of the gene product thereof is the activity of electron transport complex protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “electron transport complex protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B1627, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1627, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1627, or a functional equivalent or a homolog thereof as depicted in column 8 of Table II, application no. 17, preferably a homolog or functional equivalent as depicted in column 8 of Table II B, application no. 17, and being depicted in the same respective line as said B1627, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “electron transport complex protein”, preferably it is the molecule of section (a) or (b) of this paragraph.

In particular, it was observed that in plants, especially in Arabidopsis thaliana, increasing or generating the activity of a gene product non-targeted with the activity of a “electron transport complex protein”, preferably being encoded by a gene comprising the nucleic acid sequence SEQ ID NO. 38345, preferably the coding region thereof, conferred the production of or the increase in glucose-1-phosphate compared with the wild type control.

The nucleic acid sequence of B1627 from Escherichia coli, e.g. as shown in the respective line in column 5 of Table I, application no. 17, has been published in B. And the activity of the gene product thereof is the activity of electron transport complex protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “electron transport complex protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B1627, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1627, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1627, or a functional equivalent or a homolog thereof as depicted in column 8 of Table II, application no. 17, preferably a homolog or functional equivalent as depicted in column 8 of Table II B, application no. 17, and being depicted in the same respective line as said B1627, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “electron transport complex protein”, preferably it is the molecule of section (a) or (b) of this paragraph.

In particular, it was observed that in plants, especially in Arabidopsis thaliana, increasing or generating the activity of a gene product non-targeted with the activity of a “electron transport complex protein”, preferably being encoded by a gene comprising the nucleic acid sequence SEQ ID NO. 38345, preferably the coding region thereof, conferred the production of or the increase in 3,4,5-trihydroxypentanoic acid compared with the wild type control.

The nucleic acid sequence of B1651 from Escherichia coli, e.g. as shown in the respective line in column 5 of Table I, application no. 17, has been published in B. And the activity of the gene product thereof is the activity of methylglyoxalase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “methylglyoxalase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B1651, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1651, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1651, or a functional equivalent or a homolog thereof as depicted in column 8 of Table II, application no. 17, preferably a homolog or functional equivalent as depicted in column 8 of Table II B, application no. 17, and being depicted in the same respective line as said B1651, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “methylglyoxalase”, preferably it is the molecule of section (a) or (b) of this paragraph.

In particular, it was observed that in plants, especially in Arabidopsis thaliana, increasing or generating the activity of a gene product non-targeted with the activity of a “methylglyoxalase”, preferably being encoded by a gene comprising the nucleic acid sequence SEQ ID NO. 78265, preferably the coding region thereof, conferred the production of or the increase in myo-inositol compared with the wild type control.

The nucleic acid sequence of B1670 from Escherichia coli, e.g. as shown in the respective line in column 5 of Table I, application no. 17, has been published in B. And the activity of the gene product thereof is the activity of b1670-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “b1670-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B1670, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1670, or a functional equivalent or a homolog thereof as depicted in column 8 of Table II, application no. 17, preferably a homolog or functional equivalent as depicted in column 8 of Table II B, application no. 17, and being depicted in the same respective line as said B1670, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “b1670-protein”, preferably it is the molecule of section (a) or (b) of this paragraph.

In particular, it was observed that in plants, especially in Arabidopsis thaliana, increasing or generating the activity of a gene product non-targeted with the activity of a “b1670-protein”, preferably being encoded by a gene comprising the nucleic acid sequence SEQ ID NO. 78753, preferably the coding region thereof, conferred the production of or the increase in myo-inositol compared with the wild type control.

The nucleic acid sequence of B1755 from Escherichia coli, e.g. as shown in the respective line in column 5 of Table I, application no. 17, has been published in B. And the activity of the gene product thereof is the activity of ABC transporter permease protein.

Accordingly, in one embodiment, the process of the present invention for producing fructose in a non-human organism, like a microorganism or a plant or a part thereof, comprises increasing or generating the activity of a gene product with the activity of a gene product conferring the activity of “ABC transporter permease protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B1755, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1755, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1755, or a functional equivalent or a homolog thereof as depicted in column 8 of Table II, application no. 17, preferably a homolog or functional equivalent as depicted in column 8 of Table II B, application no. 17, and being depicted in the same respective line as said B1755, and preferably the activity is increased non-targeted, whereby the respective line disclose in column 7 the fine chemical fructose.

Accordingly, in one embodiment, the molecule which activity is to be increased in the process of the invention is the gene product with an activity as a “ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “ABC transporter permease protein”, preferably being encoded by a gene comprising the nucleic acid sequence SEQ ID NO. 78852, preferably the coding region thereof, conferred the production of or the increase in fructose compared with the wild type control.

The nucleic acid sequence of B1791 from Escherichia coli, e.g. as shown in the respective line in column 5 of Table I, application no. 17, has been published in B. And the activity of the gene product thereof is the activity of transport protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “transport protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B1791, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1791, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1791, or a functional equivalent or a homolog thereof as depicted in column 8 of Table II, application no. 17, preferably a homolog or functional equivalent as depicted in column 8 of Table II B, application no. 17, and being depicted in the same respective line as said B1791, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “transport protein”, preferably it is the molecule of section (a) or (b) of this paragraph.

In particular, it was observed that in plants, especially in Arabidopsis thaliana, increasing or generating the activity of a gene product non-targeted with the activity of a “transport protein”, preferably being encoded by a gene comprising the nucleic acid sequence SEQ ID NO. 78883, preferably the coding region thereof, conferred the production of or the increase in myo-inositol compared with the wild type control.

The nucleic acid sequence of B1796 from Escherichia coli, e.g. as shown in the respective line in column 5 of Table I, application no. 17, has been published in B. And the activity of the gene product thereof is the activity of b1796-protein.

Accordingly, in one embodiment, the process of the present invention for producing fructose in a non-human organism, like a microorganism or a plant or a part thereof, comprises increasing or generating the activity of a gene product with the activity of a gene product conferring the activity of “b1796-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B1796, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1796, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1796, or a functional equivalent or a homolog thereof as depicted in column 8 of Table II, application no. 17, preferably a homolog or functional equivalent as depicted in column 8 of Table II B, application no. 17, and being depicted in the same respective line as said B1796, and preferably the activity is increased non-targeted, whereby the respective line disclose in column 7 the fine chemical fructose.

Accordingly, in one embodiment, the molecule which activity is to be increased in the process of the invention is the gene product with an activity as a “b1796-protein”, preferably it is the molecule of section (a) or (b) of this paragraph.

In particular, it was observed that in plants, especially in Arabidopsis thaliana, increasing or generating the activity of a gene product non-targeted with the activity of a “b1796-protein”, preferably being encoded by a gene comprising the nucleic acid sequence SEQ ID NO. 99196, preferably the coding region thereof, conferred the production of or the increase in fructose compared with the wild type control.

The nucleic acid sequence of B1812 from Escherichia coli, e.g. as shown in the respective line in column 5 of Table I, application no. 17, has been published in B. And the activity of the gene product thereof is the activity of para-aminobenzoate synthase component I.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “para-aminobenzoate synthase componentl”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B1812, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1812, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1812, or a functional equivalent or a homolog thereof as depicted in column 8 of Table II, application no. 17, preferably a homolog or functional equivalent as depicted in column 8 of Table II B, application no. 17, and being depicted in the same respective line as said B1812, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “para-aminobenzoate synthase componentl”, preferably it is the molecule of section (a) or (b) of this paragraph.

In particular, it was observed that in plants, especially in Arabidopsis thaliana, increasing or generating the activity of a gene product non-targeted with the activity of a “paraaminobenzoate synthase componentl”, preferably being encoded by a gene comprising the nucleic acid sequence SEQ ID NO. 145180, preferably the coding region thereof, conferred the production of or the increase in myo-inositol compared with the wild type control.

The nucleic acid sequence of B1814 from Escherichia coli, e.g. as shown in the respective line in column 5 of Table I, application no. 17, has been published in B. And the activity of the gene product thereof is the activity of L-serine dehydratase.

Accordingly, in one embodiment, the process of the present invention for producing galactitol in a non-human organism, like a microorganism or a plant or a part thereof, comprises increasing or generating the activity of a gene product with the activity of a gene product conferring the activity of “L-serine dehydratase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical galactitol), application no. 17, preferably the coding region thereof, or a homolog or a fragment thereof, and being depicted in the same respective line as said B1814, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1814, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1814, or a functional equivalent or a homolog thereof as depicted in column 8 of Table II, application no. 17, preferably a homolog or functional equivalent as depicted in column 8 of Table II B, application no. 17, and being depicted in the same respective line as said B1814, and preferably the activity is increased plastidic, whereby the respective line disclose in column 7 the fine chemical galactitol.

Accordingly, in one embodiment, the molecule which activity is to be increased in the process of the invention is the gene product with an activity as a “L-serine dehydratase”, preferably it is the molecule of section (a) or (b) of this paragraph.

In particular, it was observed that in plants, especially in Arabidopsis thaliana, increasing or generating the activity of a gene product plastidic with the activity of a “L-serine dehydratase”, preferably being encoded by a gene comprising the nucleic acid sequence SEQ ID NO. 8033, preferably the coding region thereof, conferred the production of or the increase in galactitol compared with the wild type control.

The nucleic acid sequence of B1814 from Escherichia coli, e.g. as shown in the respective line in column 5 of Table I, application no. 17, has been published in B. And the activity of the gene product thereof is the activity of L-serine dehydratase.

Accordingly, in one embodiment, the process of the present invention for producing sorbitol in a non-human organism, like a microorganism or a plant or a part thereof, comprises increasing or generating the activity of a gene product with the activity of a gene product conferring the activity of “L-serine dehydratase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical sorbitol), application no. 17, preferably the coding region thereof, or a homolog or a fragment thereof, and being depicted in the same respective line as said B1814, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1814, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1814, or a functional equivalent or a homolog thereof as depicted in column 8 of Table II, application no. 17, preferably a homolog or functional equivalent as depicted in column 8 of Table II B, application no. 17, and being depicted in the same respective line as said B1814, and preferably the activity is increased plastidic, whereby the respective line disclose in column 7 the fine chemical sorbitol.

Accordingly, in one embodiment, the molecule which activity is to be increased in the process of the invention is the gene product with an activity as a “L-serine dehydratase”, preferably it is the molecule of section (a) or (b) of this paragraph.

In particular, it was observed that in plants, especially in Arabidopsis thaliana, increasing or generating the activity of a gene product plastidic with the activity of a “L-serine dehydratase”, preferably being encoded by a gene comprising the nucleic acid sequence SEQ ID NO. 8033, preferably the coding region thereof, conferred the production of or the increase in sorbitol compared with the wild type control.

The nucleic acid sequence of B1845 from Escherichia coli, e.g. as shown in the respective line in column 5 of Table I, application no. 17, has been published in B. And the activity of the gene product thereof is the activity of protease.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “protease”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B1845, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1845, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1845, or a functional equivalent or a homolog thereof as depicted in column 8 of Table II, application no. 17, preferably a homolog or functional equivalent as depicted in column 8 of Table II B, application no. 17, and being depicted in the same respective line as said B1845, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “protease”, preferably it is the molecule of section (a) or (b) of this paragraph.

In particular, it was observed that in plants, especially in Arabidopsis thaliana, increasing or generating the activity of a gene product non-targeted with the activity of a “protease”, preferably being encoded by a gene comprising the nucleic acid sequence SEQ ID NO. 38573, preferably the coding region thereof, conferred the production of or the increase in myo-inositol compared with the wild type control.

The nucleic acid sequence of B1854 from Escherichia coli, e.g. as shown in the respective line in column 5 of Table I, application no. 17, has been published in B. And the activity of the gene product thereof is the activity of pyruvate kinase.

Accordingly, in one embodiment, the process of the present invention for producing erythrol in a non-human organism, like a microorganism or a plant or a part thereof, comprises increasing or generating the activity of a gene product with the activity of a gene product conferring the activity of “pyruvate kinase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical erythrol), application no. 17, preferably the coding region thereof, or a homolog or a fragment thereof, and being depicted in the same respective line as said B1854, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1854, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1854, or a functional equivalent or a homolog thereof as depicted in column 8 of Table II, application no. 17, preferably a homolog or functional equivalent as depicted in column 8 of Table II B, application no. 17, and being depicted in the same respective line as said B1854, and preferably the activity is increased plastidic, whereby the respective line disclose in column 7 the fine chemical erythrol.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 8363, preferably the coding region thereof, conferred the production of or the increase in erythrol compared with the wild type control.

The nucleic acid sequence of B1854 from Escherichia coli, e.g. as shown in the respective line in column 5 of Table I, application no. 17, has been published in B. And the activity of the gene product thereof is the activity of pyruvate kinase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “pyruvate kinase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B1854, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1854, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1854, or a functional equivalent or a homolog thereof as depicted in column 8 of Table II, application no. 17, preferably a homolog or functional equivalent as depicted in column 8 of Table II B, application no. 17, and being depicted in the same respective line as said B1854, and preferably the activity is increased plastidic, whereby the respective line disclose in column 7 the fine chemical myo-inositol.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 8363, preferably the coding region thereof, conferred the production of or the increase in myo-inositol compared with the wild type control.

The nucleic acid sequence of B1854 from Escherichia coli, e.g. as shown in the respective line in column 5 of Table I, application no. 17, has been published in B. And the activity of the gene product thereof is the activity of pyruvate kinase.

Accordingly, in one embodiment, the process of the present invention for producing sorbitol in a non-human organism, like a microorganism or a plant or a part thereof, comprises increasing or generating the activity of a gene product with the activity of a gene product conferring the activity of “pyruvate kinase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical sorbitol), application no. 17, preferably the coding region thereof, or a homolog or a fragment thereof, and being depicted in the same respective line as said B1854, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1854, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1854, or a functional equivalent or a homolog thereof as depicted in column 8 of Table II, application no. 17, preferably a homolog or functional equivalent as depicted in column 8 of Table II B, application no. 17, and being depicted in the same respective line as said B1854, and preferably the activity is increased plastidic, whereby the respective line disclose in column 7 the fine chemical sorbitol.

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 8363, preferably the coding region thereof, conferred the production of or the increase in sorbitol compared with the wild type control.

The nucleic acid sequence of B1865 from Escherichia coli, e.g. as shown in the respective line in column 5 of Table I, application no. 17, has been published in B. And the activity of the gene product thereof is the activity of dATP pyrophosphohydrolase.

Accordingly, in one embodiment, the process of the present invention for producing fructose in a non-human organism, like a microorganism or a plant or a part thereof, comprises increasing or generating the activity of a gene product with the activity of a gene product conferring the activity of “dATP pyrophosphohydrolase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B1865, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1865, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1865, or a functional equivalent or a homolog thereof as depicted in column 8 of Table II, application no. 17, preferably a homolog or functional equivalent as depicted in column 8 of Table II B, application no. 17, and being depicted in the same respective line as said B1865, and preferably the activity is increased non-targeted, whereby the respective line disclose in column 7 the fine chemical fructose.

Accordingly, in one embodiment, the molecule which activity is to be increased in the process of the invention is the gene product with an activity as a “dATP pyrophosphohydrolase”, preferably it is the molecule of section (a) or (b) of this paragraph.

In particular, it was observed that in plants, especially in Arabidopsis thaliana, increasing or generating the activity of a gene product non-targeted with the activity of a “dATP pyrophosphohydrolase”, preferably being encoded by a gene comprising the nucleic acid sequence SEQ ID NO. 134783, preferably the coding region thereof, conferred the production of or the increase in fructose compared with the wild type control.

The nucleic acid sequence of B1896 from Escherichia coli, e.g. as shown in the respective line in column 5 of Table I, application no. 17, has been published in B. And the activity of the gene product thereof is the activity of trehalose-6-phosphate synthase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “trehalose-6-phosphate synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B1896, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1896, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1896, or a functional equivalent or a homolog thereof as depicted in column 8 of Table II, application no. 17, preferably a homolog or functional equivalent as depicted in column 8 of Table II B, application no. 17, and being depicted in the same respective line as said B1896, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “trehalose-6-phosphate synthase”, preferably it is the molecule of section (a) or (b) of this paragraph.

In particular, it was observed that in plants, especially in Arabidopsis thaliana, increasing or generating the activity of a gene product non-targeted with the activity of a “trehalose-6-phosphate synthase”, preferably being encoded by a gene comprising the nucleic acid sequence SEQ ID NO. 145411, preferably the coding region thereof, conferred the production of or the increase in glucose-1-phosphate compared with the wild type control.

The nucleic acid sequence of B1896 from Escherichia coli, e.g. as shown in the respective line in column 5 of Table I, application no. 17, has been published in B. And the activity of the gene product thereof is the activity of trehalose-6-phosphate synthase.

Accordingly, in one embodiment, the process of the present invention for producing trehalose in a non-human organism, like a microorganism or a plant or a part thereof, comprises increasing or generating the activity of a gene product with the activity of a gene product conferring the activity of “trehalose-6-phosphate synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical trehalose), application no. 17, preferably the coding region thereof, or a homolog or a fragment thereof, and being depicted in the same respective line as said B1896, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1896, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1896, or a functional equivalent or a homolog thereof as depicted in column 8 of Table II, application no. 17, preferably a homolog or functional equivalent as depicted in column 8 of Table II B, application no. 17, and being depicted in the same respective line as said B1896, and preferably the activity is increased non-targeted, whereby the respective line disclose in column 7 the fine chemical trehalose.

Accordingly, in one embodiment, the molecule which activity is to be increased in the process of the invention is the gene product with an activity as a “trehalose-6-phosphate synthase”, preferably it is the molecule of section (a) or (b) of this paragraph.

In particular, it was observed that in plants, especially in Arabidopsis thaliana, increasing or generating the activity of a gene product non-targeted with the activity of a “trehalose-6-phosphate synthase”, preferably being encoded by a gene comprising the nucleic acid sequence SEQ ID NO. 145411, preferably the coding region thereof, conferred the production of or the increase in trehalose compared with the wild type control.

The nucleic acid sequence of B1897 from Escherichia coli, e.g. as shown in the respective line in column 5 of Table I, application no. 17, has been published in B. And the activity of the gene product thereof is the activity of trehalose-phosphatase.

Accordingly, in one embodiment, the process of the present invention for producing erythrol in a non-human organism, like a microorganism or a plant or a part thereof, comprises increasing or generating the activity of a gene product with the activity of a gene product conferring the activity of “trehalose-phosphatase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical erythrol), application no. 17, preferably the coding region thereof, or a homolog or a fragment thereof, and being depicted in the same respective line as said B1897, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1897, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1897, or a functional equivalent or a homolog thereof as depicted in column 8 of Table II, application no. 17, preferably a homolog or functional equivalent as depicted in column 8 of Table II B, application no. 17, and being depicted in the same respective line as said B1897, and preferably the activity is increased plastidic, whereby the respective line disclose in column 7 the fine chemical erythrol.

Accordingly, in one embodiment, the molecule which activity is to be increased in the process of the invention is the gene product with an activity as a “trehalose-phosphatase”, preferably it is the molecule of section (a) or (b) of this paragraph.

In particular, it was observed that in plants, especially in Arabidopsis thaliana, increasing or generating the activity of a gene product plastidic with the activity of a “trehalose-phosphatase”, preferably being encoded by a gene comprising the nucleic acid sequence SEQ ID NO. 79056, preferably the coding region thereof, conferred the production of or the increase in erythrol compared with the wild type control.

The nucleic acid sequence of B1897 from Escherichia coli, e.g. as shown in the respective line in column 5 of Table I, application no. 17, has been published in B. And the activity of the gene product thereof is the activity of trehalose-phosphatase.

Accordingly, in one embodiment, the process of the present invention for producing fructose in a non-human organism, like a microorganism or a plant or a part thereof, comprises increasing or generating the activity of a gene product with the activity of a gene product conferring the activity of “trehalose-phosphatase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B1897, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1897, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1897, or a functional equivalent or a homolog thereof as depicted in column 8 of Table II, application no. 17, preferably a homolog or functional equivalent as depicted in column 8 of Table II B, application no. 17, and being depicted in the same respective line as said B1897, and preferably the activity is increased non-targeted, whereby the respective line disclose in column 7 the fine chemical fructose.

Accordingly, in one embodiment, the molecule which activity is to be increased in the process of the invention is the gene product with an activity as a “trehalose-phosphatase”, preferably it is the molecule of section (a) or (b) of this paragraph.

In particular, it was observed that in plants, especially in Arabidopsis thaliana, increasing or generating the activity of a gene product non-targeted with the activity of a “trehalosephosphatase”, preferably being encoded by a gene comprising the nucleic acid sequence SEQ ID NO. 79056, preferably the coding region thereof, conferred the production of or the increase in fructose compared with the wild type control.

The nucleic acid sequence of B1897 from Escherichia coli, e.g. as shown in the respective line in column 5 of Table I, application no. 17, has been published in B. And the activity of the gene product thereof is the activity of trehalose-phosphatase.

Accordingly, in one embodiment, the process of the present invention for producing glucose in a non-human organism, like a microorganism or a plant or a part thereof, comprises increasing or generating the activity of a gene product with the activity of a gene product conferring the activity of “trehalose-phosphatase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B1897, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1897, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1897, or a functional equivalent or a homolog thereof as depicted in column 8 of Table II, application no. 17, preferably a homolog or functional equivalent as depicted in column 8 of Table II B, application no. 17, and being depicted in the same respective line as said B1897, and preferably the activity is increased non-targeted, whereby the respective line disclose in column 7 the fine chemical glucose.

Accordingly, in one embodiment, the molecule which activity is to be increased in the process of the invention is the gene product with an activity as a “trehalose-phosphatase”, preferably it is the molecule of section (a) or (b) of this paragraph.

In particular, it was observed that in plants, especially in Arabidopsis thaliana, increasing or generating the activity of a gene product non-targeted with the activity of a “trehalosephosphatase”, preferably being encoded by a gene comprising the nucleic acid sequence SEQ ID NO. 79056, preferably the coding region thereof, conferred the production of or the increase in glucose compared with the wild type control.

The nucleic acid sequence of B1897 from Escherichia coli, e.g. as shown in the respective line in column 5 of Table I, application no. 17, has been published in B. And the activity of the gene product thereof is the activity of trehalose-phosphatase.

Accordingly, in one embodiment, the process of the present invention for producing sucrose in a non-human organism, like a microorganism or a plant or a part thereof, comprises increasing or generating the activity of a gene product with the activity of a gene product conferring the activity of “trehalose-phosphatase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said B1897, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1897, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1897, or a functional equivalent or a homolog thereof as depicted in column 8 of Table II, application no. 17, preferably a homolog or functional equivalent as depicted in column 8 of Table II B, application no. 17, and being depicted in the same respective line as said B1897, and preferably the activity is increased non-targeted, whereby the respective line disclose in column 7 the fine chemical sucrose.

Accordingly, in one embodiment, the molecule which activity is to be increased in the process of the invention is the gene product with an activity as a “trehalose-phosphatase”, preferably it is the molecule of section (a) or (b) of this paragraph.

In particular, it was observed that in plants, especially in Arabidopsis thaliana, increasing or generating the activity of a gene product non-targeted with the activity of a “trehalosephosphatase”, preferably being encoded by a gene comprising the nucleic acid sequence SEQ ID NO. 79056, preferably the coding region thereof, conferred the production of or the increase in sucrose compared with the wild type control.

The nucleic acid sequence of B1898 from Escherichia coli, e.g. as shown in the respective line in column 5 of Table I, application no. 17, has been published in B. And the activity of the gene product thereof is the activity of b1898-protein.

Accordingly, in one embodiment, the process of the present invention for producing raffinose in a non-human organism, like a microorganism or a plant or a part thereof, comprises increasing or generating the activity of a gene product with the activity of a gene product conferring the activity of “b1898-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said B1898, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1898, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1898, or a functional equivalent or a homolog thereof as depicted in column 8 of Table II, application no. 17, preferably a homolog or functional equivalent as depicted in column 8 of Table II B, application no. 17, and being depicted in the same respective line as said B1898, and preferably the activity is increased non-targeted, whereby the respective line disclose in column 7 the fine chemical raffinose.

Accordingly, in one embodiment, the molecule which activity is to be increased in the process of the invention is the gene product with an activity as a “b1898-protein”, preferably it is the molecule of section (a) or (b) of this paragraph.

In particular, it was observed that in plants, especially in Arabidopsis thaliana, increasing or generating the activity of a gene product non-targeted with the activity of a “b1898-protein”, preferably being encoded by a gene comprising the nucleic acid sequence SEQ ID NO. 38767, preferably the coding region thereof, conferred the production of or the increase in raffinose compared with the wild type control.

The nucleic acid sequence of B1932 from Escherichia coli, e.g. as shown in the respective line in column 5 of Table I, application no. 17, has been published in B. And the activity of the gene product thereof is the activity of acetyltransferase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B1932, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1932, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1932, or a functional equivalent or a homolog thereof as depicted in column 8 of Table II, application no. 17, preferably a homolog or functional equivalent as depicted in column 8 of Table II B, application no. 17, and being depicted in the same respective line as said B1932, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 131799, preferably the coding region thereof, conferred the production of or the increase in glucose-1-phosphate compared with the wild type control.

The nucleic acid sequence of B1932 from Escherichia coli, e.g. as shown in the respective line in column 5 of Table I, application no. 17, has been published in B. And the activity of the gene product thereof is the activity of acetyltransferase.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B1932, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1932, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1932, or a functional equivalent or a homolog thereof as depicted in column 8 of Table II, application no. 17, preferably a homolog or functional equivalent as depicted in column 8 of Table II B, application no. 17, and being depicted in the same respective line as said B1932, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 131799, preferably the coding region thereof, conferred the production of or the increase in 3,4,5-trihydroxypentanoic acid compared with the wild type control.

The nucleic acid sequence of B1938 from Escherichia coli, e.g. as shown in the respective line in column 5 of Table I, application no. 17, has been published in B. And the activity of the gene product thereof is the activity of flagellar M-ring protein.

Accordingly, in one embodiment, the process of the present invention for producing glucose in a non-human organism, like a microorganism or a plant or a part thereof, comprises increasing or generating the activity of a gene product with the activity of a gene product conferring the activity of “flagellar M-ring protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B1938, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1938, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1938, or a functional equivalent or a homolog thereof as depicted in column 8 of Table II, application no. 17, preferably a homolog or functional equivalent as depicted in column 8 of Table II B, application no. 17, and being depicted in the same respective line as said B1938, and preferably the activity is increased non-targeted, whereby the respective line disclose in column 7 the fine chemical glucose.

Accordingly, in one embodiment, the molecule which activity is to be increased in the process of the invention is the gene product with an activity as a “flagellar M-ring protein”, preferably it is the molecule of section (a) or (b) of this paragraph.

In particular, it was observed that in plants, especially in Arabidopsis thaliana, increasing or generating the activity of a gene product non-targeted with the activity of a “flagellar M-ring protein”, preferably being encoded by a gene comprising the nucleic acid sequence SEQ ID NO. 134864, preferably the coding region thereof, conferred the production of or the increase in glucose compared with the wild type control.

The nucleic acid sequence of B1938 from Escherichia coli, e.g. as shown in the respective line in column 5 of Table I, application no. 17, has been published in B. And the activity of the gene product thereof is the activity of flagellar M-ring protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “flagellar M-ring protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B1938, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B1938, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B1938, or a functional equivalent or a homolog thereof as depicted in column 8 of Table II, application no. 17, preferably a homolog or functional equivalent as depicted in column 8 of Table II B, application no. 17, and being depicted in the same respective line as said B1938, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “flagellar M-ring protein”, preferably it is the molecule of section (a) or (b) of this paragraph.

In particular, it was observed that in plants, especially in Arabidopsis thaliana, increasing or generating the activity of a gene product non-targeted with the activity of a “flagellar M-ring protein”, preferably being encoded by a gene comprising the nucleic acid sequence SEQ ID NO. 134864, preferably the coding region thereof, conferred the production of or the increase in myo-inositol compared with the wild type control.

The nucleic acid sequence of B2027 from Escherichia coli, e.g. as shown in the respective line in column 5 of Table I, application no. 17, has been published in B. And the activity of the gene product thereof is the activity of O-antigen chain length determinant.

Accordingly, in one embodiment, the process of the present invention for producing fructose in a non-human organism, like a microorganism or a plant or a part thereof, comprises increasing or generating the activity of a gene product with the activity of a gene product conferring the activity of “O-antigen chain length determinant”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B2027, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2027, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2027, or a functional equivalent or a homolog thereof as depicted in column 8 of Table II, application no. 17, preferably a homolog or functional equivalent as depicted in column 8 of Table II B, application no. 17, and being depicted in the same respective line as said B2027, and preferably the activity is increased non-targeted, whereby the respective line disclose in column 7 the fine chemical fructose.

Accordingly, in one embodiment, the molecule which activity is to be increased in the process of the invention is the gene product with an activity as a “O-antigen chain length determinant”, preferably it is the molecule of section (a) or (b) of this paragraph.

In particular, it was observed that in plants, especially in Arabidopsis thaliana, increasing or generating the activity of a gene product non-targeted with the activity of a “O-antigen chain length determinant”, preferably being encoded by a gene comprising the nucleic acid sequence SEQ ID NO. 79217, preferably the coding region thereof, conferred the production of or the increase in fructose compared with the wild type control.

The nucleic acid sequence of B2032 from Escherichia coli, e.g. as shown in the respective line in column 5 of Table I, application no. 17, has been published in B. And the activity of the gene product thereof is the activity of b2032-protein.

Accordingly, in one embodiment, the process of the present invention for producing glucose in a non-human organism, like a microorganism or a plant or a part thereof, comprises increasing or generating the activity of a gene product with the activity of a gene product conferring the activity of “b2032-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B2032, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2032, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2032, or a functional equivalent or a homolog thereof as depicted in column 8 of Table II, application no. 17, preferably a homolog or functional equivalent as depicted in column 8 of Table II B, application no. 17, and being depicted in the same respective line as said B2032, and preferably the activity is increased non-targeted, whereby the respective line disclose in column 7 the fine chemical glucose.

Accordingly, in one embodiment, the molecule which activity is to be increased in the process of the invention is the gene product with an activity as a “b2032-protein”, preferably it is the molecule of section (a) or (b) of this paragraph.

In particular, it was observed that in plants, especially in Arabidopsis thaliana, increasing or generating the activity of a gene product non-targeted with the activity of a “b2032-protein”, preferably being encoded by a gene comprising the nucleic acid sequence SEQ ID NO. 8920, preferably the coding region thereof, conferred the production of or the increase in glucose compared with the wild type control.

The nucleic acid sequence of B2032 from Escherichia coli, e.g. as shown in the respective line in column 5 of Table I, application no. 17, has been published in B. And the activity of the gene product thereof is the activity of b2032-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “b2032-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B2032, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2032, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2032, or a functional equivalent or a homolog thereof as depicted in column 8 of Table II, application no. 17, preferably a homolog or functional equivalent as depicted in column 8 of Table II B, application no. 17, and being depicted in the same respective line as said B2032, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “b2032-protein”, preferably it is the molecule of section (a) or (b) of this paragraph.

In particular, it was observed that in plants, especially in Arabidopsis thaliana, increasing or generating the activity of a gene product non-targeted with the activity of a “b2032-protein”, preferably being encoded by a gene comprising the nucleic acid sequence SEQ ID NO. 8920, preferably the coding region thereof, conferred the production of or the increase in myo-inositol compared with the wild type control.

The nucleic acid sequence of B2043 from Escherichia coli, e.g. as shown in the respective line in column 5 of Table I, application no. 17, has been published in B. And the activity of the gene product thereof is the activity of colanic acid biosynthesis protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “colanic acid biosynthesis protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B2043, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2043, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2043, or a functional equivalent or a homolog thereof as depicted in column 8 of Table II, application no. 17, preferably a homolog or functional equivalent as depicted in column 8 of Table II B, application no. 17, and being depicted in the same respective line as said B2043, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “colanic acid biosynthesis protein”, preferably it is the molecule of section (a) or (b) of this paragraph.

In particular, it was observed that in plants, especially in Arabidopsis thaliana, increasing or generating the activity of a gene product non-targeted with the activity of a “colanic acid biosynthesis protein”, preferably being encoded by a gene comprising the nucleic acid sequence SEQ ID NO. 112193, preferably the coding region thereof, conferred the production of or the increase in glucose-1-phosphate compared with the wild type control.

The nucleic acid sequence of B2053 from Escherichia coli, e.g. as shown in the respective line in column 5 of Table I, application no. 17, has been published in B. And the activity of the gene product thereof is the activity of GDP-mannose 4,6-dehydratase.

Accordingly, in one embodiment, the process of the present invention for producing fucose in a non-human organism, like a microorganism or a plant or a part thereof, comprises increasing or generating the activity of a gene product with the activity of a gene product conferring the activity of “GDP-mannose 4,6-dehydratase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fucose), application no. 17, preferably the coding region thereof, or a homolog or a fragment thereof, and being depicted in the same respective line as said B2053, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2053, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2053, or a functional equivalent or a homolog thereof as depicted in column 8 of Table II, application no. 17, preferably a homolog or functional equivalent as depicted in column 8 of Table II B, application no. 17, and being depicted in the same respective line as said B2053, and preferably the activity is increased non-targeted, whereby the respective line disclose in column 7 the fine chemical fucose.

Accordingly, in one embodiment, the molecule which activity is to be increased in the process of the invention is the gene product with an activity as a “GDP-mannose 4,6-dehydratase”, preferably it is the molecule of section (a) or (b) of this paragraph.

In particular, it was observed that in plants, especially in Arabidopsis thaliana, increasing or generating the activity of a gene product non-targeted with the activity of a “GDP-mannose 4,6-dehydratase”, preferably being encoded by a gene comprising the nucleic acid sequence SEQ ID NO. 104277, preferably the coding region thereof, conferred the production of or the increase in fucose compared with the wild type control.

The nucleic acid sequence of B2063 from Escherichia coli, e.g. as shown in the respective line in column 5 of Table I, application no. 17, has been published in B. And the activity of the gene product thereof is the activity of transport protein.

Accordingly, in one embodiment, the process of the present invention for producing fructose in a non-human organism, like a microorganism or a plant or a part thereof, comprises increasing or generating the activity of a gene product with the activity of a gene product conferring the activity of “transport protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B2063, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2063, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2063, or a functional equivalent or a homolog thereof as depicted in column 8 of Table II, application no. 17, preferably a homolog or functional equivalent as depicted in column 8 of Table II B, application no. 17, and being depicted in the same respective line as said B2063, and preferably the activity is increased non-targeted, whereby the respective line disclose in column 7 the fine chemical fructose.

Accordingly, in one embodiment, the molecule which activity is to be increased in the process of the invention is the gene product with an activity as a “transport protein”, preferably it is the molecule of section (a) or (b) of this paragraph.

In particular, it was observed that in plants, especially in Arabidopsis thaliana, increasing or generating the activity of a gene product non-targeted with the activity of a “transport protein”, preferably being encoded by a gene comprising the nucleic acid sequence SEQ ID NO. 38947, preferably the coding region thereof, conferred the production of or the increase in fructose compared with the wild type control.

The nucleic acid sequence of B2063 from Escherichia coli, e.g. as shown in the respective line in column 5 of Table I, application no. 17, has been published in B. And the activity of the gene product thereof is the activity of transport protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “transport protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B2063, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2063, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2063, or a functional equivalent or a homolog thereof as depicted in column 8 of Table II, application no. 17, preferably a homolog or functional equivalent as depicted in column 8 of Table II B, application no. 17, and being depicted in the same respective line as said B2063, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “transport protein”, preferably it is the molecule of section (a) or (b) of this paragraph.

In particular, it was observed that in plants, especially in Arabidopsis thaliana, increasing or generating the activity of a gene product non-targeted with the activity of a “transport protein”, preferably being encoded by a gene comprising the nucleic acid sequence SEQ ID NO. 38947, preferably the coding region thereof, conferred the production of or the increase in glucose-1-phosphate compared with the wild type control.

The nucleic acid sequence of B2063 from Escherichia coli, e.g. as shown in the respective line in column 5 of Table I, application no. 17, has been published in B. And the activity of the gene product thereof is the activity of transport protein.

Accordingly, in one embodiment, the process of the present invention for producing raffinose in a non-human organism, like a microorganism or a plant or a part thereof, comprises increasing or generating the activity of a gene product with the activity of a gene product conferring the activity of “transport protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said B2063, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2063, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2063, or a functional equivalent or a homolog thereof as depicted in column 8 of Table II, application no. 17, preferably a homolog or functional equivalent as depicted in column 8 of Table II B, application no. 17, and being depicted in the same respective line as said B2063, and preferably the activity is increased non-targeted, whereby the respective line disclose in column 7 the fine chemical raffinose.

Accordingly, in one embodiment, the molecule which activity is to be increased in the process of the invention is the gene product with an activity as a “transport protein”, preferably it is the molecule of section (a) or (b) of this paragraph.

In particular, it was observed that in plants, especially in Arabidopsis thaliana, increasing or generating the activity of a gene product non-targeted with the activity of a “transport protein”, preferably being encoded by a gene comprising the nucleic acid sequence SEQ ID NO. 38947, preferably the coding region thereof, conferred the production of or the increase in raffinose compared with the wild type control.

The nucleic acid sequence of B2063 from Escherichia coli, e.g. as shown in the respective line in column 5 of Table I, application no. 17, has been published in B. And the activity of the gene product thereof is the activity of transport protein.

Accordingly, in one embodiment, the process of the present invention for producing sucrose in a non-human organism, like a microorganism or a plant or a part thereof, comprises increasing or generating the activity of a gene product with the activity of a gene product conferring the activity of “transport protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said B2063, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2063, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2063, or a functional equivalent or a homolog thereof as depicted in column 8 of Table II, application no. 17, preferably a homolog or functional equivalent as depicted in column 8 of Table II B, application no. 17, and being depicted in the same respective line as said B2063, and preferably the activity is increased non-targeted, whereby the respective line disclose in column 7 the fine chemical sucrose.

Accordingly, in one embodiment, the molecule which activity is to be increased in the process of the invention is the gene product with an activity as a “transport protein”, preferably it is the molecule of section (a) or (b) of this paragraph.

In particular, it was observed that in plants, especially in Arabidopsis thaliana, increasing or generating the activity of a gene product non-targeted with the activity of a “transport protein”, preferably being encoded by a gene comprising the nucleic acid sequence SEQ ID NO. 38947, preferably the coding region thereof, conferred the production of or the increase in sucrose compared with the wild type control.

The nucleic acid sequence of B2063 from Escherichia coli, e.g. as shown in the respective line in column 5 of Table I, application no. 17, has been published in B. And the activity of the gene product thereof is the activity of transport protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “transport protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B2063, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2063, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2063, or a functional equivalent or a homolog thereof as depicted in column 8 of Table II, application no. 17, preferably a homolog or functional equivalent as depicted in column 8 of Table II B, application no. 17, and being depicted in the same respective line as said B2063, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “transport protein”, preferably it is the molecule of section (a) or (b) of this paragraph.

In particular, it was observed that in plants, especially in Arabidopsis thaliana, increasing or generating the activity of a gene product non-targeted with the activity of a “transport protein”, preferably being encoded by a gene comprising the nucleic acid sequence SEQ ID NO. 38947, preferably the coding region thereof, conferred the production of or the increase in 3,4,5-trihydroxypentanoic acid compared with the wild type control.

The nucleic acid sequence of B2107 from Escherichia coli, e.g. as shown in the respective line in column 5 of Table I, application no. 17, has been published in B. And the activity of the gene product thereof is the activity of b2107-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “b2107-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B2107, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2107, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2107, or a functional equivalent or a homolog thereof as depicted in column 8 of Table II, application no. 17, preferably a homolog or functional equivalent as depicted in column 8 of Table II B, application no. 17, and being depicted in the same respective line as said B2107, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “b2107-protein”, preferably it is the molecule of section (a) or (b) of this paragraph.

In particular, it was observed that in plants, especially in Arabidopsis thaliana, increasing or generating the activity of a gene product non-targeted with the activity of a “b2107-protein”, preferably being encoded by a gene comprising the nucleic acid sequence SEQ ID NO. 39002, preferably the coding region thereof, conferred the production of or the increase in glucose-1-phosphate compared with the wild type control.

The nucleic acid sequence of B2107 from Escherichia coli, e.g. as shown in the respective line in column 5 of Table I, application no. 17, has been published in B. And the activity of the gene product thereof is the activity of b2107-protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “b2107-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B2107, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2107, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2107, or a functional equivalent or a homolog thereof as depicted in column 8 of Table II, application no. 17, preferably a homolog or functional equivalent as depicted in column 8 of Table II B, application no. 17, and being depicted in the same respective line as said B2107, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “b2107-protein”, preferably it is the molecule of section (a) or (b) of this paragraph.

In particular, it was observed that in plants, especially in Arabidopsis thaliana, increasing or generating the activity of a gene product non-targeted with the activity of a “b2107-protein”, preferably being encoded by a gene comprising the nucleic acid sequence SEQ ID NO. 39002, preferably the coding region thereof, conferred the production of or the increase in 3,4,5-trihydroxypentanoic acid compared with the wild type control.

The nucleic acid sequence of B2139 from Escherichia coli, e.g. as shown in the respective line in column 5 of Table I, application no. 17, has been published in B. And the activity of the gene product thereof is the activity of b2139-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “b2139-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B2139, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2139, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2139, or a functional equivalent or a homolog thereof as depicted in column 8 of Table II, application no. 17, preferably a homolog or functional equivalent as depicted in column 8 of Table II B, application no. 17, and being depicted in the same respective line as said B2139, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “b2139-protein”, preferably it is the molecule of section (a) or (b) of this paragraph.

In particular, it was observed that in plants, especially in Arabidopsis thaliana, increasing or generating the activity of a gene product plastidic with the activity of a “b2139-protein”, preferably being encoded by a gene comprising the nucleic acid sequence SEQ ID NO. 125085, preferably the coding region thereof, conferred the production of or the increase in glucose-1-phosphate compared with the wild type control.

The nucleic acid sequence of B2242 from Escherichia coli, e.g. as shown in the respective line in column 5 of Table I, application no. 17, has been published in B. And the activity of the gene product thereof is the activity of sn-glycerol-3-phosphate dehydrogenase.

Accordingly, in one embodiment, the process of the present invention for producing erythrol in a non-human organism, like a microorganism or a plant or a part thereof, comprises increasing or generating the activity of a gene product with the activity of a gene product conferring the activity of “sn-glycerol-3-phosphate dehydrogenase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical erythrol), application no. 17, preferably the coding region thereof, or a homolog or a fragment thereof, and being depicted in the same respective line as said B2242, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2242, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2242, or a functional equivalent or a homolog thereof as depicted in column 8 of Table II, application no. 17, preferably a homolog or functional equivalent as depicted in column 8 of Table II B, application no. 17, and being depicted in the same respective line as said B2242, and preferably the activity is increased plastidic, whereby the respective line disclose in column 7 the fine chemical erythrol.

Accordingly, in one embodiment, the molecule which activity is to be increased in the process of the invention is the gene product with an activity as a “sn-glycerol-3-phosphate dehydrogenase”, preferably it is the molecule of section (a) or (b) of this paragraph.

In particular, it was observed that in plants, especially in Arabidopsis thaliana, increasing or generating the activity of a gene product plastidic with the activity of a “sn-glycerol-3-phosphate dehydrogenase”, preferably being encoded by a gene comprising the nucleic acid sequence SEQ ID NO. 79279, preferably the coding region thereof, conferred the production of or the increase in erythrol compared with the wild type control.

The nucleic acid sequence of B2285 from Escherichia coli, e.g. as shown in the respective line in column 5 of Table I, application no. 17, has been published in B. And the activity of the gene product thereof is the activity of ATP synthase subunit E.

Accordingly, in one embodiment, the process of the present invention for producing fructose in a non-human organism, like a microorganism or a plant or a part thereof, comprises increasing or generating the activity of a gene product with the activity of a gene product conferring the activity of “ATP synthase subunit E”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B2285, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2285, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2285, or a functional equivalent or a homolog thereof as depicted in column 8 of Table II, application no. 17, preferably a homolog or functional equivalent as depicted in column 8 of Table II B, application no. 17, and being depicted in the same respective line as said B2285, and preferably the activity is increased non-targeted, whereby the respective line disclose in column 7 the fine chemical fructose.

Accordingly, in one embodiment, the molecule which activity is to be increased in the process of the invention is the gene product with an activity as a “ATP synthase subunit E”, preferably it is the molecule of section (a) or (b) of this paragraph.

In particular, it was observed that in plants, especially in Arabidopsis thaliana, increasing or generating the activity of a gene product non-targeted with the activity of a “ATP synthase subunit E”, preferably being encoded by a gene comprising the nucleic acid sequence SEQ ID NO. 99620, preferably the coding region thereof, conferred the production of or the increase in fructose compared with the wild type control.

The nucleic acid sequence of B2290 from Escherichia coli, e.g. as shown in the respective line in column 5 of Table I, application no. 17, has been published in B. And the activity of the gene product thereof is the activity of aminotransferase.

Accordingly, in one embodiment, the process of the present invention for producing galactinol in a non-human organism, like a microorganism or a plant or a part thereof, comprises increasing or generating the activity of a gene product with the activity of a gene product conferring the activity of “aminotransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical galactinol), application no. 17, preferably the coding region thereof, or a homolog or a fragment thereof, and being depicted in the same respective line as said B2290, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2290, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2290, or a functional equivalent or a homolog thereof as depicted in column 8 of Table II, application no. 17, preferably a homolog or functional equivalent as depicted in column 8 of Table II B, application no. 17, and being depicted in the same respective line as said B2290, and preferably the activity is increased non-targeted, whereby the respective line disclose in column 7 the fine chemical galactinol.

Accordingly, in one embodiment, the molecule which activity is to be increased in the process of the invention is the gene product with an activity as a “aminotransferase”, preferably it is the molecule of section (a) or (b) of this paragraph.

In particular, it was observed that in plants, especially in Arabidopsis thaliana, increasing or generating the activity of a gene product non-targeted with the activity of a “aminotransferase”, preferably being encoded by a gene comprising the nucleic acid sequence SEQ ID NO. 145692, preferably the coding region thereof, conferred the production of or the increase in galactinol compared with the wild type control.

The nucleic acid sequence of B2345 from Escherichia coli, e.g. as shown in the respective line in column 5 of Table I, application no. 17, has been published in B. And the activity of the gene product thereof is the activity of b2345-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “b2345-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B2345, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2345, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2345, or a functional equivalent or a homolog thereof as depicted in column 8 of Table II, application no. 17, preferably a homolog or functional equivalent as depicted in column 8 of Table II B, application no. 17, and being depicted in the same respective line as said B2345, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “b2345-protein”, preferably it is the molecule of section (a) or (b) of this paragraph.

In particular, it was observed that in plants, especially in Arabidopsis thaliana, increasing or generating the activity of a gene product non-targeted with the activity of a “b2345-protein”, preferably being encoded by a gene comprising the nucleic acid sequence SEQ ID NO. 9156, preferably the coding region thereof, conferred the production of or the increase in myo-inositol compared with the wild type control.

The nucleic acid sequence of B2345 from Escherichia coli, e.g. as shown in the respective line in column 5 of Table I, application no. 17, has been published in B. And the activity of the gene product thereof is the activity of b2345-protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “b2345-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B2345, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2345, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2345, or a functional equivalent or a homolog thereof as depicted in column 8 of Table II, application no. 17, preferably a homolog or functional equivalent as depicted in column 8 of Table II B, application no. 17, and being depicted in the same respective line as said B2345, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “b2345-protein”, preferably it is the molecule of section (a) or (b) of this paragraph.

In particular, it was observed that in plants, especially in Arabidopsis thaliana, increasing or generating the activity of a gene product non-targeted with the activity of a “b2345-protein”, preferably being encoded by a gene comprising the nucleic acid sequence SEQ ID NO. 9156, preferably the coding region thereof, conferred the production of or the increase in 3,4,5-trihydroxypentanoic acid compared with the wild type control.

The nucleic acid sequence of B2360 from Escherichia coli, e.g. as shown in the respective line in column 5 of Table I, application no. 17, has been published in B. And the activity of the gene product thereof is the activity of b2360-protein.

Accordingly, in one embodiment, the process of the present invention for producing verbascose in a non-human organism, like a microorganism or a plant or a part thereof, comprises increasing or generating the activity of a gene product with the activity of a gene product conferring the activity of “b2360-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical verbascose), application no. 17, preferably the coding region thereof, or a homolog or a fragment thereof, and being depicted in the same respective line as said B2360, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2360, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2360, or a functional equivalent or a homolog thereof as depicted in column 8 of Table II, application no. 17, preferably a homolog or functional equivalent as depicted in column 8 of Table II B, application no. 17, and being depicted in the same respective line as said B2360, and preferably the activity is increased non-targeted, whereby the respective line disclose in column 7 the fine chemical verbascose.

Accordingly, in one embodiment, the molecule which activity is to be increased in the process of the invention is the gene product with an activity as a “b2360-protein”, preferably it is the molecule of section (a) or (b) of this paragraph.

In particular, it was observed that in plants, especially in Arabidopsis thaliana, increasing or generating the activity of a gene product non-targeted with the activity of a “b2360-protein”, preferably being encoded by a gene comprising the nucleic acid sequence SEQ ID NO. 39219, preferably the coding region thereof, conferred the production of or the increase in verbascose compared with the wild type control.

The nucleic acid sequence of B2360 from Escherichia coli, e.g. as shown in the respective line in column 5 of Table I, application no. 17, has been published in B. And the activity of the gene product thereof is the activity of b2360-protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “b2360-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B2360, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2360, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2360, or a functional equivalent or a homolog thereof as depicted in column 8 of Table II, application no. 17, preferably a homolog or functional equivalent as depicted in column 8 of Table II B, application no. 17, and being depicted in the same respective line as said B2360, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “b2360-protein”, preferably it is the molecule of section (a) or (b) of this paragraph.

In particular, it was observed that in plants, especially in Arabidopsis thaliana, increasing or generating the activity of a gene product non-targeted with the activity of a “b2360-protein”, preferably being encoded by a gene comprising the nucleic acid sequence SEQ ID NO. 39219, preferably the coding region thereof, conferred the production of or the increase in 3,4,5-trihydroxypentanoic acid compared with the wild type control.

The nucleic acid sequence of B2361 from Escherichia coli, e.g. as shown in the respective line in column 5 of Table I, application no. 17, has been published in B. And the activity of the gene product thereof is the activity of b2361-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “b2361-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B2361, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2361, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2361, or a functional equivalent or a homolog thereof as depicted in column 8 of Table II, application no. 17, preferably a homolog or functional equivalent as depicted in column 8 of Table II B, application no. 17, and being depicted in the same respective line as said B2361, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “b2361-protein”, preferably it is the molecule of section (a) or (b) of this paragraph.

In particular, it was observed that in plants, especially in Arabidopsis thaliana, increasing or generating the activity of a gene product non-targeted with the activity of a “b2361-protein”, preferably being encoded by a gene comprising the nucleic acid sequence SEQ ID NO. 146204, preferably the coding region thereof, conferred the production of or the increase in glucose-1-phosphate compared with the wild type control.

The nucleic acid sequence of B2361 from Escherichia coli, e.g. as shown in the respective line in column 5 of Table I, application no. 17, has been published in B. And the activity of the gene product thereof is the activity of b2361-protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “b2361-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B2361, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2361, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2361, or a functional equivalent or a homolog thereof as depicted in column 8 of Table II, application no. 17, preferably a homolog or functional equivalent as depicted in column 8 of Table II B, application no. 17, and being depicted in the same respective line as said B2361, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “b2361-protein”, preferably it is the molecule of section (a) or (b) of this paragraph.

In particular, it was observed that in plants, especially in Arabidopsis thaliana, increasing or generating the activity of a gene product non-targeted with the activity of a “b2361-protein”, preferably being encoded by a gene comprising the nucleic acid sequence SEQ ID NO. 146204, preferably the coding region thereof, conferred the production of or the increase in 3,4,5-trihydroxypentanoic acid compared with the wild type control.

The nucleic acid sequence of B2389 from Escherichia coli, e.g. as shown in the respective line in column 5 of Table I, application no. 17, has been published in B. And the activity of the gene product thereof is the activity of ion-transport protein.

Accordingly, in one embodiment, the process of the present invention for producing sucrose in a non-human organism, like a microorganism or a plant or a part thereof, comprises increasing or generating the activity of a gene product with the activity of a gene product conferring the activity of “ion-transport protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said B2389, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2389, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2389, or a functional equivalent or a homolog thereof as depicted in column 8 of Table II, application no. 17, preferably a homolog or functional equivalent as depicted in column 8 of Table II B, application no. 17, and being depicted in the same respective line as said B2389, and preferably the activity is increased non-targeted, whereby the respective line disclose in column 7 the fine chemical sucrose.

Accordingly, in one embodiment, the molecule which activity is to be increased in the process of the invention is the gene product with an activity as a “ion-transport protein”, preferably it is the molecule of section (a) or (b) of this paragraph.

In particular, it was observed that in plants, especially in Arabidopsis thaliana, increasing or generating the activity of a gene product non-targeted with the activity of a “ion-transport protein”, preferably being encoded by a gene comprising the nucleic acid sequence SEQ ID NO. 146238, preferably the coding region thereof, conferred the production of or the increase in sucrose compared with the wild type control.

The nucleic acid sequence of B2399 from Escherichia coli, e.g. as shown in the respective line in column 5 of Table I, application no. 17, has been published in B. And the activity of the gene product thereof is the activity of b2399-protein.

Accordingly, in one embodiment, the process of the present invention for producing fructose in a non-human organism, like a microorganism or a plant or a part thereof, comprises increasing or generating the activity of a gene product with the activity of a gene product conferring the activity of “b2399-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B2399, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2399, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2399, or a functional equivalent or a homolog thereof as depicted in column 8 of Table II, application no. 17, preferably a homolog or functional equivalent as depicted in column 8 of Table II B, application no. 17, and being depicted in the same respective line as said B2399, and preferably the activity is increased non-targeted, whereby the respective line disclose in column 7 the fine chemical fructose.

Accordingly, in one embodiment, the molecule which activity is to be increased in the process of the invention is the gene product with an activity as a “b2399-protein”, preferably it is the molecule of section (a) or (b) of this paragraph.

In particular, it was observed that in plants, especially in Arabidopsis thaliana, increasing or generating the activity of a gene product non-targeted with the activity of a “b2399-protein”, preferably being encoded by a gene comprising the nucleic acid sequence SEQ ID NO. 39237, preferably the coding region thereof, conferred the production of or the increase in fructose compared with the wild type control.

The nucleic acid sequence of B2399 from Escherichia coli, e.g. as shown in the respective line in column 5 of Table I, application no. 17, has been published in B. And the activity of the gene product thereof is the activity of b2399-protein.

Accordingly, in one embodiment, the process of the present invention for producing glucose in a non-human organism, like a microorganism or a plant or a part thereof, comprises increasing or generating the activity of a gene product with the activity of a gene product conferring the activity of “b2399-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B2399, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2399, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2399, or a functional equivalent or a homolog thereof as depicted in column 8 of Table II, application no. 17, preferably a homolog or functional equivalent as depicted in column 8 of Table II B, application no. 17, and being depicted in the same respective line as said B2399, and preferably the activity is increased non-targeted, whereby the respective line disclose in column 7 the fine chemical glucose.

Accordingly, in one embodiment, the molecule which activity is to be increased in the process of the invention is the gene product with an activity as a “b2399-protein”, preferably it is the molecule of section (a) or (b) of this paragraph.

In particular, it was observed that in plants, especially in Arabidopsis thaliana, increasing or generating the activity of a gene product non-targeted with the activity of a “b2399-protein”, preferably being encoded by a gene comprising the nucleic acid sequence SEQ ID NO. 39237, preferably the coding region thereof, conferred the production of or the increase in glucose compared with the wild type control.

The nucleic acid sequence of B2399 from Escherichia coli, e.g. as shown in the respective line in column 5 of Table I, application no. 17, has been published in B. And the activity of the gene product thereof is the activity of b2399-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “b2399-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B2399, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2399, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2399, or a functional equivalent or a homolog thereof as depicted in column 8 of Table II, application no. 17, preferably a homolog or functional equivalent as depicted in column 8 of Table II B, application no. 17, and being depicted in the same respective line as said B2399, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “b2399-protein”, preferably it is the molecule of section (a) or (b) of this paragraph.

In particular, it was observed that in plants, especially in Arabidopsis thaliana, increasing or generating the activity of a gene product non-targeted with the activity of a “b2399-protein”, preferably being encoded by a gene comprising the nucleic acid sequence SEQ ID NO. 39237, preferably the coding region thereof, conferred the production of or the increase in glucose-1-phosphate compared with the wild type control.

The nucleic acid sequence of B2399 from Escherichia coli, e.g. as shown in the respective line in column 5 of Table I, application no. 17, has been published in B. And the activity of the gene product thereof is the activity of b2399-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “b2399-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B2399, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2399, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2399, or a functional equivalent or a homolog thereof as depicted in column 8 of Table II, application no. 17, preferably a homolog or functional equivalent as depicted in column 8 of Table II B, application no. 17, and being depicted in the same respective line as said B2399, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “b2399-protein”, preferably it is the molecule of section (a) or (b) of this paragraph.

In particular, it was observed that in plants, especially in Arabidopsis thaliana, increasing or generating the activity of a gene product non-targeted with the activity of a “b2399-protein”, preferably being encoded by a gene comprising the nucleic acid sequence SEQ ID NO. 39237, preferably the coding region thereof, conferred the production of or the increase in myo-inositol compared with the wild type control.

The nucleic acid sequence of B2399 from Escherichia coli, e.g. as shown in the respective line in column 5 of Table I, application no. 17, has been published in B. And the activity of the gene product thereof is the activity of b2399-protein.

Accordingly, in one embodiment, the process of the present invention for producing raffinose in a non-human organism, like a microorganism or a plant or a part thereof, comprises increasing or generating the activity of a gene product with the activity of a gene product conferring the activity of “b2399-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said B2399, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2399, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2399, or a functional equivalent or a homolog thereof as depicted in column 8 of Table II, application no. 17, preferably a homolog or functional equivalent as depicted in column 8 of Table II B, application no. 17, and being depicted in the same respective line as said B2399, and preferably the activity is increased non-targeted, whereby the respective line disclose in column 7 the fine chemical raffinose.

Accordingly, in one embodiment, the molecule which activity is to be increased in the process of the invention is the gene product with an activity as a “b2399-protein”, preferably it is the molecule of section (a) or (b) of this paragraph.

In particular, it was observed that in plants, especially in Arabidopsis thaliana, increasing or generating the activity of a gene product non-targeted with the activity of a “b2399-protein”, preferably being encoded by a gene comprising the nucleic acid sequence SEQ ID NO. 39237, preferably the coding region thereof, conferred the production of or the increase in raffinose compared with the wild type control.

The nucleic acid sequence of B2399 from Escherichia coli, e.g. as shown in the respective line in column 5 of Table I, application no. 17, has been published in B. And the activity of the gene product thereof is the activity of b2399-protein.

Accordingly, in one embodiment, the process of the present invention for producing sedoheptulose-7-phosphate in a non-human organism, like a microorganism or a plant or a part thereof, comprises increasing or generating the activity of a gene product with the activity of a gene product conferring the activity of “b2399-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical sedoheptulose-7-phosphate), application no. 17, preferably the coding region thereof, or a homolog or a fragment thereof, and being depicted in the same respective line as said B2399, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2399, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2399, or a functional equivalent or a homolog thereof as depicted in column 8 of Table II, application no. 17, preferably a homolog or functional equivalent as depicted in column 8 of Table II B, application no. 17, and being depicted in the same respective line as said B2399, and preferably the activity is increased non-targeted, whereby the respective line disclose in column 7 the fine chemical sedoheptulose-7-phosphate.

Accordingly, in one embodiment, the molecule which activity is to be increased in the process of the invention is the gene product with an activity as a “b2399-protein”, preferably it is the molecule of section (a) or (b) of this paragraph.

In particular, it was observed that in plants, especially in Arabidopsis thaliana, increasing or generating the activity of a gene product non-targeted with the activity of a “b2399-protein”, preferably being encoded by a gene comprising the nucleic acid sequence SEQ ID NO. 39237, preferably the coding region thereof, conferred the production of or the increase in sedoheptulose-7-phosphate compared with the wild type control.

The nucleic acid sequence of B2399 from Escherichia coli, e.g. as shown in the respective line in column 5 of Table I, application no. 17, has been published in B. And the activity of the gene product thereof is the activity of b2399-protein.

Accordingly, in one embodiment, the process of the present invention for producing sucrose in a non-human organism, like a microorganism or a plant or a part thereof, comprises increasing or generating the activity of a gene product with the activity of a gene product conferring the activity of “b2399-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said B2399, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2399, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2399, or a functional equivalent or a homolog thereof as depicted in column 8 of Table II, application no. 17, preferably a homolog or functional equivalent as depicted in column 8 of Table II B, application no. 17, and being depicted in the same respective line as said B2399, and preferably the activity is increased non-targeted, whereby the respective line disclose in column 7 the fine chemical sucrose.

Accordingly, in one embodiment, the molecule which activity is to be increased in the process of the invention is the gene product with an activity as a “b2399-protein”, preferably it is the molecule of section (a) or (b) of this paragraph.

In particular, it was observed that in plants, especially in Arabidopsis thaliana, increasing or generating the activity of a gene product non-targeted with the activity of a “b2399-protein”, preferably being encoded by a gene comprising the nucleic acid sequence SEQ ID NO. 39237, preferably the coding region thereof, conferred the production of or the increase in sucrose compared with the wild type control.

The nucleic acid sequence of B2405 from Escherichia coli, e.g. as shown in the respective line in column 5 of Table I, application no. 17, has been published in B. And the activity of the gene product thereof is the activity of transcriptional regulator.

Accordingly, in one embodiment, the process of the present invention for producing fructose in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B2405, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2405, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2405, or a functional equivalent or a homolog thereof as depicted in column 8 of Table II, application no. 17, preferably a homolog or functional equivalent as depicted in column 8 of Table II B, application no. 17, and being depicted in the same respective line as said B2405, and preferably the activity is increased non-targeted, whereby the respective line disclose in column 7 the fine chemical fructose.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 39255, preferably the coding region thereof, conferred the production of or the increase in fructose compared with the wild type control.

The nucleic acid sequence of B2405 from Escherichia coli, e.g. as shown in the respective line in column 5 of Table I, application no. 17, has been published in B. And the activity of the gene product thereof is the activity of transcriptional regulator.

Accordingly, in one embodiment, the process of the present invention for producing raffinose in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said B2405, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2405, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2405, or a functional equivalent or a homolog thereof as depicted in column 8 of Table II, application no. 17, preferably a homolog or functional equivalent as depicted in column 8 of Table II B, application no. 17, and being depicted in the same respective line as said B2405, and preferably the activity is increased non-targeted, whereby the respective line disclose in column 7 the fine chemical raffinose.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 39255, preferably the coding region thereof, conferred the production of or the increase in raffinose compared with the wild type control.

The nucleic acid sequence of B2414 from Escherichia coli, e.g. as shown in the respective line in column 5 of Table I, application no. 17, has been published in B. And the activity of the gene product thereof is the activity of cysteine synthase A.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “cysteine synthase A”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B2414, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2414, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2414, or a functional equivalent or a homolog thereof as depicted in column 8 of Table II, application no. 17, preferably a homolog or functional equivalent as depicted in column 8 of Table II B, application no. 17, and being depicted in the same respective line as said B2414, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “cysteine synthase A”, preferably it is the molecule of section (a) or (b) of this paragraph.

In particular, it was observed that in plants, especially in Arabidopsis thaliana, increasing or generating the activity of a gene product non-targeted with the activity of a “cysteine synthase A”, preferably being encoded by a gene comprising the nucleic acid sequence SEQ ID NO. 39300, preferably the coding region thereof, conferred the production of or the increase in myo-inositol compared with the wild type control.

The nucleic acid sequence of B2414 from Escherichia coli, e.g. as shown in the respective line in column 5 of Table I, application no. 17, has been published in B. And the activity of the gene product thereof is the activity of cysteine synthase A.

Accordingly, in one embodiment, the process of the present invention for producing verbascose in a non-human organism, like a microorganism or a plant or a part thereof, comprises increasing or generating the activity of a gene product with the activity of a gene product conferring the activity of “cysteine synthase A”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical verbascose), application no. 17, preferably the coding region thereof, or a homolog or a fragment thereof, and being depicted in the same respective line as said B2414, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2414, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2414, or a functional equivalent or a homolog thereof as depicted in column 8 of Table II, application no. 17, preferably a homolog or functional equivalent as depicted in column 8 of Table II B, application no. 17, and being depicted in the same respective line as said B2414, and preferably the activity is increased non-targeted, whereby the respective line disclose in column 7 the fine chemical verbascose.

Accordingly, in one embodiment, the molecule which activity is to be increased in the process of the invention is the gene product with an activity as a “cysteine synthase A”, preferably it is the molecule of section (a) or (b) of this paragraph.

In particular, it was observed that in plants, especially in Arabidopsis thaliana, increasing or generating the activity of a gene product non-targeted with the activity of a “cysteine synthase A”, preferably being encoded by a gene comprising the nucleic acid sequence SEQ ID NO. 39300, preferably the coding region thereof, conferred the production of or the increase in verbascose compared with the wild type control.

The nucleic acid sequence of B2466 from Escherichia coli, e.g. as shown in the respective line in column 5 of Table I, application no. 17, has been published in B. And the activity of the gene product thereof is the activity of b2466-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “b2466-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B2466, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2466, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2466, or a functional equivalent or a homolog thereof as depicted in column 8 of Table II, application no. 17, preferably a homolog or functional equivalent as depicted in column 8 of Table II B, application no. 17, and being depicted in the same respective line as said B2466, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “b2466-protein”, preferably it is the molecule of section (a) or (b) of this paragraph.

In particular, it was observed that in plants, especially in Arabidopsis thaliana, increasing or generating the activity of a gene product non-targeted with the activity of a “b2466-protein”, preferably being encoded by a gene comprising the nucleic acid sequence SEQ ID NO. 146261, preferably the coding region thereof, conferred the production of or the increase in glucose-1-phosphate compared with the wild type control.

The nucleic acid sequence of B2466 from Escherichia coli, e.g. as shown in the respective line in column 5 of Table I, application no. 17, has been published in B. And the activity of the gene product thereof is the activity of b2466-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “b2466-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B2466, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2466, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2466, or a functional equivalent or a homolog thereof as depicted in column 8 of Table II, application no. 17, preferably a homolog or functional equivalent as depicted in column 8 of Table II B, application no. 17, and being depicted in the same respective line as said B2466, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “b2466-protein”, preferably it is the molecule of section (a) or (b) of this paragraph.

In particular, it was observed that in plants, especially in Arabidopsis thaliana, increasing or generating the activity of a gene product non-targeted with the activity of a “b2466-protein”, preferably being encoded by a gene comprising the nucleic acid sequence SEQ ID NO. 146261, preferably the coding region thereof, conferred the production of or the increase in myo-inositol compared with the wild type control.

The nucleic acid sequence of B2466 from Escherichia coli, e.g. as shown in the respective line in column 5 of Table I, application no. 17, has been published in B. And the activity of the gene product thereof is the activity of b2466-protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “b2466-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B2466, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2466, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2466, or a functional equivalent or a homolog thereof as depicted in column 8 of Table II, application no. 17, preferably a homolog or functional equivalent as depicted in column 8 of Table II B, application no. 17, and being depicted in the same respective line as said B2466, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “b2466-protein”, preferably it is the molecule of section (a) or (b) of this paragraph.

In particular, it was observed that in plants, especially in Arabidopsis thaliana, increasing or generating the activity of a gene product non-targeted with the activity of a “b2466-protein”, preferably being encoded by a gene comprising the nucleic acid sequence SEQ ID NO. 146261, preferably the coding region thereof, conferred the production of or the increase in 3,4,5-trihydroxypentanoic acid compared with the wild type control.

The nucleic acid sequence of B2474 from Escherichia coli, e.g. as shown in the respective line in column 5 of Table I, application no. 17, has been published in B. And the activity of the gene product thereof is the activity of b2474-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “b2474-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B2474, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2474, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2474, or a functional equivalent or a homolog thereof as depicted in column 8 of Table II, application no. 17, preferably a homolog or functional equivalent as depicted in column 8 of Table II B, application no. 17, and being depicted in the same respective line as said B2474, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “b2474-protein”, preferably it is the molecule of section (a) or (b) of this paragraph.

In particular, it was observed that in plants, especially in Arabidopsis thaliana, increasing or generating the activity of a gene product non-targeted with the activity of a “b2474-protein”, preferably being encoded by a gene comprising the nucleic acid sequence SEQ ID NO. 40329, preferably the coding region thereof, conferred the production of or the increase in glucose-1-phosphate compared with the wild type control.

The nucleic acid sequence of B2541 from Escherichia coli, e.g. as shown in the respective line in column 5 of Table I, application no. 17, has been published in B. And the activity of the gene product thereof is the activity of 2,3-dihydroxy-2,3-dihydrophenylpropionatedehydrogenase.

Accordingly, in one embodiment, the process of the present invention for producing raffinose in a non-human organism, like a microorganism or a plant or a part thereof, comprises increasing or generating the activity of a gene product with the activity of a gene product conferring the activity of “2,3-dihydroxy-2,3-dihydrophenylpropionatedehydrogenase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said B2541, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2541, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2541, or a functional equivalent or a homolog thereof as depicted in column 8 of Table II, application no. 17, preferably a homolog or functional equivalent as depicted in column 8 of Table II B, application no. 17, and being depicted in the same respective line as said B2541, and preferably the activity is increased non-targeted, whereby the respective line disclose in column 7 the fine chemical raffinose.

Accordingly, in one embodiment, the molecule which activity is to be increased in the process of the invention is the gene product with an activity as a “2,3-dihydroxy-2,3-dihydrophenylpropionatedehydrogenase”, preferably it is the molecule of section (a) or (b) of this paragraph.

In particular, it was observed that in plants, especially in Arabidopsis thaliana, increasing or generating the activity of a gene product non-targeted with the activity of a “2,3-dihydroxy-2,3-dihydrophenylpropionatedehydrogenase”, preferably being encoded by a gene comprising the nucleic acid sequence SEQ ID NO. 40383, preferably the coding region thereof, conferred the production of or the increase in raffinose compared with the wild type control.

The nucleic acid sequence of B2546 from Escherichia coli, e.g. as shown in the respective line in column 5 of Table I, application no. 17, has been published in B. And the activity of the gene product thereof is the activity of putative transport system permease protein.

Accordingly, in one embodiment, the process of the present invention for producing sucrose in a non-human organism, like a microorganism or a plant or a part thereof, comprises increasing or generating the activity of a gene product with the activity of a gene product conferring the activity of “putative transport system permease protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said B2546, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2546, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2546, or a functional equivalent or a homolog thereof as depicted in column 8 of Table II, application no. 17, preferably a homolog or functional equivalent as depicted in column 8 of Table II B, application no. 17, and being depicted in the same respective line as said B2546, and preferably the activity is increased non-targeted, whereby the respective line disclose in column 7 the fine chemical sucrose.

Accordingly, in one embodiment, the molecule which activity is to be increased in the process of the invention is the gene product with an activity as a “putative transport system permease protein”, preferably it is the molecule of section (a) or (b) of this paragraph.

In particular, it was observed that in plants, especially in Arabidopsis thaliana, increasing or generating the activity of a gene product non-targeted with the activity of a “putative transport system permease protein”, preferably being encoded by a gene comprising the nucleic acid sequence SEQ ID NO. 99898, preferably the coding region thereof, conferred the production of or the increase in sucrose compared with the wild type control.

The nucleic acid sequence of B2613 from Escherichia coli, e.g. as shown in the respective line in column 5 of Table I, application no. 17, has been published in B. And the activity of the gene product thereof is the activity of b2613-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “b2613-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B2613, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2613, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2613, or a functional equivalent or a homolog thereof as depicted in column 8 of Table II, application no. 17, preferably a homolog or functional equivalent as depicted in column 8 of Table II B, application no. 17, and being depicted in the same respective line as said B2613, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “b2613-protein”, preferably it is the molecule of section (a) or (b) of this paragraph.

In particular, it was observed that in plants, especially in Arabidopsis thaliana, increasing or generating the activity of a gene product non-targeted with the activity of a “b2613-protein”, preferably being encoded by a gene comprising the nucleic acid sequence SEQ ID NO. 40665, preferably the coding region thereof, conferred the production of or the increase in glucose-1-phosphate compared with the wild type control.

The nucleic acid sequence of B2613 from Escherichia coli, e.g. as shown in the respective line in column 5 of Table I, application no. 17, has been published in B. And the activity of the gene product thereof is the activity of b2613-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “b2613-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B2613, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2613, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2613, or a functional equivalent or a homolog thereof as depicted in column 8 of Table II, application no. 17, preferably a homolog or functional equivalent as depicted in column 8 of Table II B, application no. 17, and being depicted in the same respective line as said B2613, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “b2613-protein”, preferably it is the molecule of section (a) or (b) of this paragraph.

In particular, it was observed that in plants, especially in Arabidopsis thaliana, increasing or generating the activity of a gene product non-targeted with the activity of a “b2613-protein”, preferably being encoded by a gene comprising the nucleic acid sequence SEQ ID NO. 40665, preferably the coding region thereof, conferred the production of or the increase in myo-inositol compared with the wild type control.

The nucleic acid sequence of B2634 from Escherichia coli, e.g. as shown in the respective line in column 5 of Table I, application no. 17, has been published in B. And the activity of the gene product thereof is the activity of transcriptional regulator.

Accordingly, in one embodiment, the process of the present invention for producing fructose in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B2634, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2634, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2634, or a functional equivalent or a homolog thereof as depicted in column 8 of Table II, application no. 17, preferably a homolog or functional equivalent as depicted in column 8 of Table II B, application no. 17, and being depicted in the same respective line as said B2634, and preferably the activity is increased non-targeted, whereby the respective line disclose in column 7 the fine chemical fructose.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 40726, preferably the coding region thereof, conferred the production of or the increase in fructose compared with the wild type control.

The nucleic acid sequence of B2634 from Escherichia coli, e.g. as shown in the respective line in column 5 of Table I, application no. 17, has been published in B. And the activity of the gene product thereof is the activity of transcriptional regulator.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B2634, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2634, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2634, or a functional equivalent or a homolog thereof as depicted in column 8 of Table II, application no. 17, preferably a homolog or functional equivalent as depicted in column 8 of Table II B, application no. 17, and being depicted in the same respective line as said B2634, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 40726, preferably the coding region thereof, conferred the production of or the increase in myo-inositol compared with the wild type control.

The nucleic acid sequence of B2634 from Escherichia coli, e.g. as shown in the respective line in column 5 of Table I, application no. 17, has been published in B. And the activity of the gene product thereof is the activity of transcriptional regulator.

Accordingly, in one embodiment, the process of the present invention for producing sucrose in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said B2634, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2634, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2634, or a functional equivalent or a homolog thereof as depicted in column 8 of Table II, application no. 17, preferably a homolog or functional equivalent as depicted in column 8 of Table II B, application no. 17, and being depicted in the same respective line as said B2634, and preferably the activity is increased non-targeted, whereby the respective line disclose in column 7 the fine chemical sucrose.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 40726, preferably the coding region thereof, conferred the production of or the increase in sucrose compared with the wild type control.

The nucleic acid sequence of B2653 from Escherichia coli, e.g. as shown in the respective line in column 5 of Table I, application no. 17, has been published in B. And the activity of the gene product thereof is the activity of b2653-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “b2653-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B2653, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2653, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2653, or a functional equivalent or a homolog thereof as depicted in column 8 of Table II, application no. 17, preferably a homolog or functional equivalent as depicted in column 8 of Table II B, application no. 17, and being depicted in the same respective line as said B2653, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “b2653-protein”, preferably it is the molecule of section (a) or (b) of this paragraph.

In particular, it was observed that in plants, especially in Arabidopsis thaliana, increasing or generating the activity of a gene product non-targeted with the activity of a “b2653-protein”, preferably being encoded by a gene comprising the nucleic acid sequence SEQ ID NO. 146288, preferably the coding region thereof, conferred the production of or the increase in glucose-1-phosphate compared with the wild type control.

The nucleic acid sequence of B2653 from Escherichia coli, e.g. as shown in the respective line in column 5 of Table I, application no. 17, has been published in B. And the activity of the gene product thereof is the activity of b2653-protein.

Accordingly, in one embodiment, the process of the present invention for producing sedoheptulose-7-phosphate in a non-human organism, like a microorganism or a plant or a part thereof, comprises increasing or generating the activity of a gene product with the activity of a gene product conferring the activity of “b2653-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical sedoheptulose-7-phosphate), application no. 17, preferably the coding region thereof, or a homolog or a fragment thereof, and being depicted in the same respective line as said B2653, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2653, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2653, or a functional equivalent or a homolog thereof as depicted in column 8 of Table II, application no. 17, preferably a homolog or functional equivalent as depicted in column 8 of Table II B, application no. 17, and being depicted in the same respective line as said B2653, and preferably the activity is increased non-targeted, whereby the respective line disclose in column 7 the fine chemical sedoheptulose-7-phosphate.

Accordingly, in one embodiment, the molecule which activity is to be increased in the process of the invention is the gene product with an activity as a “b2653-protein”, preferably it is the molecule of section (a) or (b) of this paragraph.

In particular, it was observed that in plants, especially in Arabidopsis thaliana, increasing or generating the activity of a gene product non-targeted with the activity of a “b2653-protein”, preferably being encoded by a gene comprising the nucleic acid sequence SEQ ID NO. 146288, preferably the coding region thereof, conferred the production of or the increase in sedoheptulose-7-phosphate compared with the wild type control.

The nucleic acid sequence of B2653 from Escherichia coli, e.g. as shown in the respective line in column 5 of Table I, application no. 17, has been published in B. And the activity of the gene product thereof is the activity of b2653-protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “b2653-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B2653, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2653, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2653, or a functional equivalent or a homolog thereof as depicted in column 8 of Table II, application no. 17, preferably a homolog or functional equivalent as depicted in column 8 of Table II B, application no. 17, and being depicted in the same respective line as said B2653, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “b2653-protein”, preferably it is the molecule of section (a) or (b) of this paragraph.

In particular, it was observed that in plants, especially in Arabidopsis thaliana, increasing or generating the activity of a gene product non-targeted with the activity of a “b2653-protein”, preferably being encoded by a gene comprising the nucleic acid sequence SEQ ID NO. 146288, preferably the coding region thereof, conferred the production of or the increase in 3,4,5-trihydroxypentanoic acid compared with the wild type control.

The nucleic acid sequence of B2654 from Escherichia coli, e.g. as shown in the respective line in column 5 of Table I, application no. 17, has been published in B. And the activity of the gene product thereof is the activity of b2654-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “b2654-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B2654, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2654, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2654, or a functional equivalent or a homolog thereof as depicted in column 8 of Table II, application no. 17, preferably a homolog or functional equivalent as depicted in column 8 of Table II B, application no. 17, and being depicted in the same respective line as said B2654, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “b2654-protein”, preferably it is the molecule of section (a) or (b) of this paragraph.

In particular, it was observed that in plants, especially in Arabidopsis thaliana, increasing or generating the activity of a gene product non-targeted with the activity of a “b2654-protein”, preferably being encoded by a gene comprising the nucleic acid sequence SEQ ID NO. 125226, preferably the coding region thereof, conferred the production of or the increase in glucose-1-phosphate compared with the wild type control.

The nucleic acid sequence of B2654 from Escherichia coli, e.g. as shown in the respective line in column 5 of Table I, application no. 17, has been published in B. And the activity of the gene product thereof is the activity of b2654-protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “b2654-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B2654, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2654, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2654, or a functional equivalent or a homolog thereof as depicted in column 8 of Table II, application no. 17, preferably a homolog or functional equivalent as depicted in column 8 of Table II B, application no. 17, and being depicted in the same respective line as said B2654, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “b2654-protein”, preferably it is the molecule of section (a) or (b) of this paragraph.

In particular, it was observed that in plants, especially in Arabidopsis thaliana, increasing or generating the activity of a gene product non-targeted with the activity of a “b2654-protein”, preferably being encoded by a gene comprising the nucleic acid sequence SEQ ID NO. 125226, preferably the coding region thereof, conferred the production of or the increase in 3,4,5-trihydroxypentanoic acid compared with the wild type control.

The nucleic acid sequence of B2670 from Escherichia coli, e.g. as shown in the respective line in column 5 of Table I, application no. 17, has been published in B. And the activity of the gene product thereof is the activity of b2670-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “b2670-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B2670, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2670, or a functional equivalent or a homolog thereof as depicted in column 8 of Table II, application no. 17, preferably a homolog or functional equivalent as depicted in column 8 of Table II B, application no. 17, and being depicted in the same respective line as said B2670, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “b2670-protein”, preferably it is the molecule of section (a) or (b) of this paragraph.

In particular, it was observed that in plants, especially in Arabidopsis thaliana, increasing or generating the activity of a gene product non-targeted with the activity of a “b2670-protein”, preferably being encoded by a gene comprising the nucleic acid sequence SEQ ID NO. 146294, preferably the coding region thereof, conferred the production of or the increase in glucose-1-phosphate compared with the wild type control.

The nucleic acid sequence of B2673 from Escherichia coli, e.g. as shown in the respective line in column 5 of Table I, application no. 17, has been published in B. And the activity of the gene product thereof is the activity of b2673-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “b2673-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B2673, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2673, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2673, or a functional equivalent or a homolog thereof as depicted in column 8 of Table II, application no. 17, preferably a homolog or functional equivalent as depicted in column 8 of Table II B, application no. 17, and being depicted in the same respective line as said B2673, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “b2673-protein”, preferably it is the molecule of section (a) or (b) of this paragraph.

In particular, it was observed that in plants, especially in Arabidopsis thaliana, increasing or generating the activity of a gene product non-targeted with the activity of a “b2673-protein”, preferably being encoded by a gene comprising the nucleic acid sequence SEQ ID NO. 9244, preferably the coding region thereof, conferred the production of or the increase in glucose-1-phosphate compared with the wild type control.

The nucleic acid sequence of B2673 from Escherichia coli, e.g. as shown in the respective line in column 5 of Table I, application no. 17, has been published in B. And the activity of the gene product thereof is the activity of b2673-protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “b2673-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B2673, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2673, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2673, or a functional equivalent or a homolog thereof as depicted in column 8 of Table II, application no. 17, preferably a homolog or functional equivalent as depicted in column 8 of Table II B, application no. 17, and being depicted in the same respective line as said B2673, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “b2673-protein”, preferably it is the molecule of section (a) or (b) of this paragraph.

In particular, it was observed that in plants, especially in Arabidopsis thaliana, increasing or generating the activity of a gene product non-targeted with the activity of a “b2673-protein”, preferably being encoded by a gene comprising the nucleic acid sequence SEQ ID NO. 9244, preferably the coding region thereof, conferred the production of or the increase in 3,4,5-trihydroxypentanoic acid compared with the wild type control.

The nucleic acid sequence of B2682 from Escherichia coli, e.g. as shown in the respective line in column 5 of Table I, application no. 17, has been published in B. And the activity of the gene product thereof is the activity of b2682-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “b2682-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B2682, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2682, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2682, or a functional equivalent or a homolog thereof as depicted in column 8 of Table II, application no. 17, preferably a homolog or functional equivalent as depicted in column 8 of Table II B, application no. 17, and being depicted in the same respective line as said B2682, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “b2682-protein”, preferably it is the molecule of section (a) or (b) of this paragraph.

In particular, it was observed that in plants, especially in Arabidopsis thaliana, increasing or generating the activity of a gene product non-targeted with the activity of a “b2682-protein”, preferably being encoded by a gene comprising the nucleic acid sequence SEQ ID NO. 115655, preferably the coding region thereof, conferred the production of or the increase in myo-inositol compared with the wild type control.

The nucleic acid sequence of B2699 from Escherichia coli, e.g. as shown in the respective line in column 5 of Table I, application no. 17, has been published in B. And the activity of the gene product thereof is the activity of recombinase A.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “recombinase A”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B2699, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2699, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2699, or a functional equivalent or a homolog thereof as depicted in column 8 of Table II, application no. 17, preferably a homolog or functional equivalent as depicted in column 8 of Table II B, application no. 17, and being depicted in the same respective line as said B2699, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “recombinase A”, preferably it is the molecule of section (a) or (b) of this paragraph.

In particular, it was observed that in plants, especially in Arabidopsis thaliana, increasing or generating the activity of a gene product non-targeted with the activity of a “recombinase A”, preferably being encoded by a gene comprising the nucleic acid sequence SEQ ID NO. 100105, preferably the coding region thereof, conferred the production of or the increase in glucose-1-phosphate compared with the wild type control.

The nucleic acid sequence of B2701 from Escherichia coli, e.g. as shown in the respective line in column 5 of Table I, application no. 17, has been published in B. And the activity of the gene product thereof is the activity of murein transglycosylase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “murein transglycosylase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B2701, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2701, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2701, or a functional equivalent or a homolog thereof as depicted in column 8 of Table II, application no. 17, preferably a homolog or functional equivalent as depicted in column 8 of Table II B, application no. 17, and being depicted in the same respective line as said B2701, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “murein transglycosylase”, preferably it is the molecule of section (a) or (b) of this paragraph.

In particular, it was observed that in plants, especially in Arabidopsis thaliana, increasing or generating the activity of a gene product non-targeted with the activity of a “murein transglycosylase”, preferably being encoded by a gene comprising the nucleic acid sequence SEQ ID NO. 80756, preferably the coding region thereof, conferred the production of or the increase in glucose-1-phosphate compared with the wild type control.

The nucleic acid sequence of B2714 from Escherichia coli, e.g. as shown in the respective line in column 5 of Table I, application no. 17, has been published in B. And the activity of the gene product thereof is the activity of transcriptional regulator.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B2714, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2714, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2714, or a functional equivalent or a homolog thereof as depicted in column 8 of Table II, application no. 17, preferably a homolog or functional equivalent as depicted in column 8 of Table II B, application no. 17, and being depicted in the same respective line as said B2714, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 40741, preferably the coding region thereof, conferred the production of or the increase in myo-inositol compared with the wild type control.

The nucleic acid sequence of B2739 from Escherichia coli, e.g. as shown in the respective line in column 5 of Table I, application no. 17, has been published in B. And the activity of the gene product thereof is the activity of b2739-protein.

Accordingly, in one embodiment, the process of the present invention for producing fructose in a non-human organism, like a microorganism or a plant or a part thereof, comprises increasing or generating the activity of a gene product with the activity of a gene product conferring the activity of “b2739-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B2739, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2739, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2739, or a functional equivalent or a homolog thereof as depicted in column 8 of Table II, application no. 17, preferably a homolog or functional equivalent as depicted in column 8 of Table II B, application no. 17, and being depicted in the same respective line as said B2739, and preferably the activity is increased non-targeted, whereby the respective line disclose in column 7 the fine chemical fructose.

Accordingly, in one embodiment, the molecule which activity is to be increased in the process of the invention is the gene product with an activity as a “b2739-protein”, preferably it is the molecule of section (a) or (b) of this paragraph.

In particular, it was observed that in plants, especially in Arabidopsis thaliana, increasing or generating the activity of a gene product non-targeted with the activity of a “b2739-protein”, preferably being encoded by a gene comprising the nucleic acid sequence SEQ ID NO. 80906, preferably the coding region thereof, conferred the production of or the increase in fructose compared with the wild type control.

The nucleic acid sequence of B2739 from Escherichia coli, e.g. as shown in the respective line in column 5 of Table I, application no. 17, has been published in B. And the activity of the gene product thereof is the activity of b2739-protein.

Accordingly, in one embodiment, the process of the present invention for producing glucose in a non-human organism, like a microorganism or a plant or a part thereof, comprises increasing or generating the activity of a gene product with the activity of a gene product conferring the activity of “b2739-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B2739, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2739, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2739, or a functional equivalent or a homolog thereof as depicted in column 8 of Table II, application no. 17, preferably a homolog or functional equivalent as depicted in column 8 of Table II B, application no. 17, and being depicted in the same respective line as said B2739, and preferably the activity is increased non-targeted, whereby the respective line disclose in column 7 the fine chemical glucose.

Accordingly, in one embodiment, the molecule which activity is to be increased in the process of the invention is the gene product with an activity as a “b2739-protein”, preferably it is the molecule of section (a) or (b) of this paragraph.

In particular, it was observed that in plants, especially in Arabidopsis thaliana, increasing or generating the activity of a gene product non-targeted with the activity of a “b2739-protein”, preferably being encoded by a gene comprising the nucleic acid sequence SEQ ID NO. 80906, preferably the coding region thereof, conferred the production of or the increase in glucose compared with the wild type control.

The nucleic acid sequence of B2747 from Escherichia coli, e.g. as shown in the respective line in column 5 of Table I, application no. 17, has been published in B. And the activity of the gene product thereof is the activity of 2-C-methyl-D-erythritol 4-phosphate cytidylyltransferase.

Accordingly, in one embodiment, the process of the present invention for producing glucose in a non-human organism, like a microorganism or a plant or a part thereof, comprises increasing or generating the activity of a gene product with the activity of a gene product conferring the activity of “2-C-methyl-D-erythritol 4-phosphate cytidylyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B2747, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2747, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2747, or a functional equivalent or a homolog thereof as depicted in column 8 of Table II, application no. 17, preferably a homolog or functional equivalent as depicted in column 8 of Table II B, application no. 17, and being depicted in the same respective line as said B2747, and preferably the activity is increased non-targeted, whereby the respective line disclose in column 7 the fine chemical glucose.

Accordingly, in one embodiment, the molecule which activity is to be increased in the process of the invention is the gene product with an activity as a “2-C-methyl-D-erythritol 4-phosphate cytidylyltransferase”, preferably it is the molecule of section (a) or (b) of this paragraph.

In particular, it was observed that in plants, especially in Arabidopsis thaliana, increasing or generating the activity of a gene product non-targeted with the activity of a “2-C-methyl-D-erythritol 4-phosphate cytidylyltransferase”, preferably being encoded by a gene comprising the nucleic acid sequence SEQ ID NO. 81075, preferably the coding region thereof, conferred the production of or the increase in glucose compared with the wild type control.

The nucleic acid sequence of B2747 from Escherichia coli, e.g. as shown in the respective line in column 5 of Table I, application no. 17, has been published in B. And the activity of the gene product thereof is the activity of 2-C-methyl-D-erythritol 4-phosphate cytidylyltransferase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “2-C-methyl-D-erythritol 4-phosphate cytidylyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B2747, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2747, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2747, or a functional equivalent or a homolog thereof as depicted in column 8 of Table II, application no. 17, preferably a homolog or functional equivalent as depicted in column 8 of Table II B, application no. 17, and being depicted in the same respective line as said B2747, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “2-C-methyl-D-erythritol 4-phosphate cytidylyltransferase”, preferably it is the molecule of section (a) or (b) of this paragraph.

In particular, it was observed that in plants, especially in Arabidopsis thaliana, increasing or generating the activity of a gene product non-targeted with the activity of a “2-C-methyl-D-erythritol 4-phosphate cytidylyltransferase”, preferably being encoded by a gene comprising the nucleic acid sequence SEQ ID NO. 81075, preferably the coding region thereof, conferred the production of or the increase in myo-inositol compared with the wild type control.

The nucleic acid sequence of B2747 from Escherichia coli, e.g. as shown in the respective line in column 5 of Table I, application no. 17, has been published in B. And the activity of the gene product thereof is the activity of 2-C-methyl-D-erythritol 4-phosphate cytidylyltransferase.

Accordingly, in one embodiment, the process of the present invention for producing raffinose in a non-human organism, like a microorganism or a plant or a part thereof, comprises increasing or generating the activity of a gene product with the activity of a gene product conferring the activity of “2-C-methyl-D-erythritol 4-phosphate cytidylyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said B2747, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2747, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2747, or a functional equivalent or a homolog thereof as depicted in column 8 of Table II, application no. 17, preferably a homolog or functional equivalent as depicted in column 8 of Table II B, application no. 17, and being depicted in the same respective line as said B2747, and preferably the activity is increased non-targeted, whereby the respective line disclose in column 7 the fine chemical raffinose.

Accordingly, in one embodiment, the molecule which activity is to be increased in the process of the invention is the gene product with an activity as a “2-C-methyl-D-erythritol 4-phosphate cytidylyltransferase”, preferably it is the molecule of section (a) or (b) of this paragraph.

In particular, it was observed that in plants, especially in Arabidopsis thaliana, increasing or generating the activity of a gene product non-targeted with the activity of a “2-C-methyl-D-erythritol 4-phosphate cytidylyltransferase”, preferably being encoded by a gene comprising the nucleic acid sequence SEQ ID NO. 81075, preferably the coding region thereof, conferred the production of or the increase in raffinose compared with the wild type control.

The nucleic acid sequence of B2762 from Escherichia coli, e.g. as shown in the respective line in column 5 of Table I, application no. 17, has been published in B. And the activity of the gene product thereof is the activity of phosphoadenosine phosphosulfate reductase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “phosphoadenosine phosphosulfate reductase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B2762, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2762, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2762, or a functional equivalent or a homolog thereof as depicted in column 8 of Table II, application no. 17, preferably a homolog or functional equivalent as depicted in column 8 of Table II B, application no. 17, and being depicted in the same respective line as said B2762, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “phosphoadenosine phosphosulfate reductase”, preferably it is the molecule of section (a) or (b) of this paragraph.

In particular, it was observed that in plants, especially in Arabidopsis thaliana, increasing or generating the activity of a gene product non-targeted with the activity of a “phosphoadenosine phosphosulfate reductase”, preferably being encoded by a gene comprising the nucleic acid sequence SEQ ID NO. 81451, preferably the coding region thereof, conferred the production of or the increase in glucose-1-phosphate compared with the wild type control.

The nucleic acid sequence of B2762 from Escherichia coli, e.g. as shown in the respective line in column 5 of Table I, application no. 17, has been published in B. And the activity of the gene product thereof is the activity of phosphoadenosine phosphosulfate reductase.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “phosphoadenosine phosphosulfate reductase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B2762, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2762, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2762, or a functional equivalent or a homolog thereof as depicted in column 8 of Table II, application no. 17, preferably a homolog or functional equivalent as depicted in column 8 of Table II B, application no. 17, and being depicted in the same respective line as said B2762, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “phosphoadenosine phosphosulfate reductase”, preferably it is the molecule of section (a) or (b) of this paragraph.

In particular, it was observed that in plants, especially in Arabidopsis thaliana, increasing or generating the activity of a gene product non-targeted with the activity of a “phosphoadenosine phosphosulfate reductase”, preferably being encoded by a gene comprising the nucleic acid sequence SEQ ID NO. 81451, preferably the coding region thereof, conferred the production of or the increase in 3,4,5-trihydroxypentanoic acid compared with the wild type control.

The nucleic acid sequence of B2790 from Escherichia coli, e.g. as shown in the respective line in column 5 of Table I, application no. 17, has been published in B. And the activity of the gene product thereof is the activity of flavodoxin.

Accordingly, in one embodiment, the process of the present invention for producing fructose in a non-human organism, like a microorganism or a plant or a part thereof, comprises increasing or generating the activity of a gene product with the activity of a gene product conferring the activity of “flavodoxin”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B2790, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2790, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2790, or a functional equivalent or a homolog thereof as depicted in column 8 of Table II, application no. 17, preferably a homolog or functional equivalent as depicted in column 8 of Table II B, application no. 17, and being depicted in the same respective line as said B2790, and preferably the activity is increased non-targeted, whereby the respective line disclose in column 7 the fine chemical fructose.

Accordingly, in one embodiment, the molecule which activity is to be increased in the process of the invention is the gene product with an activity as a “flavodoxin”, preferably it is the molecule of section (a) or (b) of this paragraph.

In particular, it was observed that in plants, especially in Arabidopsis thaliana, increasing or generating the activity of a gene product non-targeted with the activity of a “flavodoxin”, preferably being encoded by a gene comprising the nucleic acid sequence SEQ ID NO. 115719, preferably the coding region thereof, conferred the production of or the increase in fructose compared with the wild type control.

The nucleic acid sequence of B2790 from Escherichia coli, e.g. as shown in the respective line in column 5 of Table I, application no. 17, has been published in B. And the activity of the gene product thereof is the activity of flavodoxin.

Accordingly, in one embodiment, the process of the present invention for producing glucose in a non-human organism, like a microorganism or a plant or a part thereof, comprises increasing or generating the activity of a gene product with the activity of a gene product conferring the activity of “flavodoxin”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B2790, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2790, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2790, or a functional equivalent or a homolog thereof as depicted in column 8 of Table II, application no. 17, preferably a homolog or functional equivalent as depicted in column 8 of Table II B, application no. 17, and being depicted in the same respective line as said B2790, and preferably the activity is increased non-targeted, whereby the respective line disclose in column 7 the fine chemical glucose.

Accordingly, in one embodiment, the molecule which activity is to be increased in the process of the invention is the gene product with an activity as a “flavodoxin”, preferably it is the molecule of section (a) or (b) of this paragraph.

In particular, it was observed that in plants, especially in Arabidopsis thaliana, increasing or generating the activity of a gene product non-targeted with the activity of a “flavodoxin”, preferably being encoded by a gene comprising the nucleic acid sequence SEQ ID NO. 115719, preferably the coding region thereof, conferred the production of or the increase in glucose compared with the wild type control.

The nucleic acid sequence of B2790 from Escherichia coli, e.g. as shown in the respective line in column 5 of Table I, application no. 17, has been published in B. And the activity of the gene product thereof is the activity of flavodoxin.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “flavodoxin”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B2790, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2790, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2790, or a functional equivalent or a homolog thereof as depicted in column 8 of Table II, application no. 17, preferably a homolog or functional equivalent as depicted in column 8 of Table II B, application no. 17, and being depicted in the same respective line as said B2790, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “flavodoxin”, preferably it is the molecule of section (a) or (b) of this paragraph.

In particular, it was observed that in plants, especially in Arabidopsis thaliana, increasing or generating the activity of a gene product non-targeted with the activity of a “flavodoxin”, preferably being encoded by a gene comprising the nucleic acid sequence SEQ ID NO. 115719, preferably the coding region thereof, conferred the production of or the increase in glucose-1-phosphate compared with the wild type control.

The nucleic acid sequence of B2790 from Escherichia coli, e.g. as shown in the respective line in column 5 of Table I, application no. 17, has been published in B. And the activity of the gene product thereof is the activity of flavodoxin.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “flavodoxin”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B2790, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2790, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2790, or a functional equivalent or a homolog thereof as depicted in column 8 of Table II, application no. 17, preferably a homolog or functional equivalent as depicted in column 8 of Table II B, application no. 17, and being depicted in the same respective line as said B2790, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “flavodoxin”, preferably it is the molecule of section (a) or (b) of this paragraph.

In particular, it was observed that in plants, especially in Arabidopsis thaliana, increasing or generating the activity of a gene product non-targeted with the activity of a “flavodoxin”, preferably being encoded by a gene comprising the nucleic acid sequence SEQ ID NO. 115719, preferably the coding region thereof, conferred the production of or the increase in myo-inositol compared with the wild type control.

The nucleic acid sequence of B2790 from Escherichia coli, e.g. as shown in the respective line in column 5 of Table I, application no. 17, has been published in B. And the activity of the gene product thereof is the activity of flavodoxin.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “flavodoxin”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B2790, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2790, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2790, or a functional equivalent or a homolog thereof as depicted in column 8 of Table II, application no. 17, preferably a homolog or functional equivalent as depicted in column 8 of Table II B, application no. 17, and being depicted in the same respective line as said B2790, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “flavodoxin”, preferably it is the molecule of section (a) or (b) of this paragraph.

In particular, it was observed that in plants, especially in Arabidopsis thaliana, increasing or generating the activity of a gene product non-targeted with the activity of a “flavodoxin”, preferably being encoded by a gene comprising the nucleic acid sequence SEQ ID NO. 115719, preferably the coding region thereof, conferred the production of or the increase in 3,4,5-trihydroxypentanoic acid compared with the wild type control.

The nucleic acid sequence of B2818 from Escherichia coli, e.g. as shown in the respective line in column 5 of Table I, application no. 17, has been published in B. And the activity of the gene product thereof is the activity of amino-acid acetyltransferase.

Accordingly, in one embodiment, the process of the present invention for producing fructose in a non-human organism, like a microorganism or a plant or a part thereof, comprises increasing or generating the activity of a gene product with the activity of a gene product conferring the activity of “amino-acid acetyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B2818, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2818, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2818, or a functional equivalent or a homolog thereof as depicted in column 8 of Table II, application no. 17, preferably a homolog or functional equivalent as depicted in column 8 of Table II B, application no. 17, and being depicted in the same respective line as said B2818, and preferably the activity is increased non-targeted, whereby the respective line disclose in column 7 the fine chemical fructose.

Accordingly, in one embodiment, the molecule which activity is to be increased in the process of the invention is the gene product with an activity as a “amino-acid acetyltransferase”, preferably it is the molecule of section (a) or (b) of this paragraph.

In particular, it was observed that in plants, especially in Arabidopsis thaliana, increasing or generating the activity of a gene product non-targeted with the activity of a “amino-acid acetyltransferase”, preferably being encoded by a gene comprising the nucleic acid sequence SEQ ID NO. 81576, preferably the coding region thereof, conferred the production of or the increase in fructose compared with the wild type control.

The nucleic acid sequence of B2818 from Escherichia coli, e.g. as shown in the respective line in column 5 of Table I, application no. 17, has been published in B. And the activity of the gene product thereof is the activity of amino-acid acetyltransferase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “amino-acid acetyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B2818, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2818, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2818, or a functional equivalent or a homolog thereof as depicted in column 8 of Table II, application no. 17, preferably a homolog or functional equivalent as depicted in column 8 of Table II B, application no. 17, and being depicted in the same respective line as said B2818, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “amino-acid acetyltransferase”, preferably it is the molecule of section (a) or (b) of this paragraph.

In particular, it was observed that in plants, especially in Arabidopsis thaliana, increasing or generating the activity of a gene product non-targeted with the activity of a “amino-acid acetyltransferase”, preferably being encoded by a gene comprising the nucleic acid sequence SEQ ID NO. 81576, preferably the coding region thereof, conferred the production of or the increase in glucose-1-phosphate compared with the wild type control.

The nucleic acid sequence of B2818 from Escherichia coli, e.g. as shown in the respective line in column 5 of Table I, application no. 17, has been published in B. And the activity of the gene product thereof is the activity of amino-acid acetyltransferase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “amino-acid acetyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B2818, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2818, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2818, or a functional equivalent or a homolog thereof as depicted in column 8 of Table II, application no. 17, preferably a homolog or functional equivalent as depicted in column 8 of Table II B, application no. 17, and being depicted in the same respective line as said B2818, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “amino-acid acetyltransferase”, preferably it is the molecule of section (a) or (b) of this paragraph.

In particular, it was observed that in plants, especially in Arabidopsis thaliana, increasing or generating the activity of a gene product non-targeted with the activity of a “amino-acid acetyltransferase”, preferably being encoded by a gene comprising the nucleic acid sequence SEQ ID NO. 81576, preferably the coding region thereof, conferred the production of or the increase in myo-inositol compared with the wild type control.

The nucleic acid sequence of B2839 from Escherichia coli, e.g. as shown in the respective line in column 5 of Table I, application no. 17, has been published in B. And the activity of the gene product thereof is the activity of transcriptional regulator.

Accordingly, in one embodiment, the process of the present invention for producing raffinose in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said B2839, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2839, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2839, or a functional equivalent or a homolog thereof as depicted in column 8 of Table II, application no. 17, preferably a homolog or functional equivalent as depicted in column 8 of Table II B, application no. 17, and being depicted in the same respective line as said B2839, and preferably the activity is increased non-targeted, whereby the respective line disclose in column 7 the fine chemical raffinose.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 81935, preferably the coding region thereof, conferred the production of or the increase in raffinose compared with the wild type control.

The nucleic acid sequence of B2923 from Escherichia coli, e.g. as shown in the respective line in column 5 of Table I, application no. 17, has been published in B. And the activity of the gene product thereof is the activity of arginine exporter protein.

Accordingly, in one embodiment, the process of the present invention for producing glucose in a non-human organism, like a microorganism or a plant or a part thereof, comprises increasing or generating the activity of a gene product with the activity of a gene product conferring the activity of “arginine exporter protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B2923, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2923, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2923, or a functional equivalent or a homolog thereof as depicted in column 8 of Table II, application no. 17, preferably a homolog or functional equivalent as depicted in column 8 of Table II B, application no. 17, and being depicted in the same respective line as said B2923, and preferably the activity is increased non-targeted, whereby the respective line disclose in column 7 the fine chemical glucose.

Accordingly, in one embodiment, the molecule which activity is to be increased in the process of the invention is the gene product with an activity as a “arginine exporter protein”, preferably it is the molecule of section (a) or (b) of this paragraph.

In particular, it was observed that in plants, especially in Arabidopsis thaliana, increasing or generating the activity of a gene product non-targeted with the activity of a “arginine exporter protein”, preferably being encoded by a gene comprising the nucleic acid sequence SEQ ID NO. 9333, preferably the coding region thereof, conferred the production of or the increase in glucose compared with the wild type control.

The nucleic acid sequence of B2923 from Escherichia coli, e.g. as shown in the respective line in column 5 of Table I, application no. 17, has been published in B. And the activity of the gene product thereof is the activity of arginine exporter protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “arginine exporter protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B2923, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2923, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2923, or a functional equivalent or a homolog thereof as depicted in column 8 of Table II, application no. 17, preferably a homolog or functional equivalent as depicted in column 8 of Table II B, application no. 17, and being depicted in the same respective line as said B2923, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “arginine exporter protein”, preferably it is the molecule of section (a) or (b) of this paragraph.

In particular, it was observed that in plants, especially in Arabidopsis thaliana, increasing or generating the activity of a gene product non-targeted with the activity of a “arginine exporter protein”, preferably being encoded by a gene comprising the nucleic acid sequence SEQ ID NO. 9333, preferably the coding region thereof, conferred the production of or the increase in myo-inositol compared with the wild type control.

The nucleic acid sequence of B2932 from Escherichia coli, e.g. as shown in the respective line in column 5 of Table I, application no. 17, has been published in B. And the activity of the gene product thereof is the activity of b2932-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “b2932-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B2932, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B2932, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B2932, or a functional equivalent or a homolog thereof as depicted in column 8 of Table II, application no. 17, preferably a homolog or functional equivalent as depicted in column 8 of Table II B, application no. 17, and being depicted in the same respective line as said B2932, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “b2932-protein”, preferably it is the molecule of section (a) or (b) of this paragraph.

In particular, it was observed that in plants, especially in Arabidopsis thaliana, increasing or generating the activity of a gene product non-targeted with the activity of a “b2932-protein”, preferably being encoded by a gene comprising the nucleic acid sequence SEQ ID NO. 146327, preferably the coding region thereof, conferred the production of or the increase in myo-inositol compared with the wild type control.

The nucleic acid sequence of B3112 from Escherichia coli, e.g. as shown in the respective line in column 5 of Table I, application no. 17, has been published in B. And the activity of the gene product thereof is the activity of L-serine dehydratase.

Accordingly, in one embodiment, the process of the present invention for producing erythrol in a non-human organism, like a microorganism or a plant or a part thereof, comprises increasing or generating the activity of a gene product with the activity of a gene product conferring the activity of “L-serine dehydratase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical erythrol), application no. 17, preferably the coding region thereof, or a homolog or a fragment thereof, and being depicted in the same respective line as said B3112, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3112, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3112, or a functional equivalent or a homolog thereof as depicted in column 8 of Table II, application no. 17, preferably a homolog or functional equivalent as depicted in column 8 of Table II B, application no. 17, and being depicted in the same respective line as said B3112, and preferably the activity is increased plastidic, whereby the respective line disclose in column 7 the fine chemical erythrol.

Accordingly, in one embodiment, the molecule which activity is to be increased in the process of the invention is the gene product with an activity as a “L-serine dehydratase”, preferably it is the molecule of section (a) or (b) of this paragraph.

In particular, it was observed that in plants, especially in Arabidopsis thaliana, increasing or generating the activity of a gene product plastidic with the activity of a “L-serine dehydratase”, preferably being encoded by a gene comprising the nucleic acid sequence SEQ ID NO. 104660, preferably the coding region thereof, conferred the production of or the increase in erythrol compared with the wild type control.

The nucleic acid sequence of B3151 from Escherichia coli, e.g. as shown in the respective line in column 5 of Table I, application no. 17, has been published in B. And the activity of the gene product thereof is the activity of b3151-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “b3151-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B3151, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3151, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3151, or a functional equivalent or a homolog thereof as depicted in column 8 of Table II, application no. 17, preferably a homolog or functional equivalent as depicted in column 8 of Table II B, application no. 17, and being depicted in the same respective line as said B3151, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “b3151-protein”, preferably it is the molecule of section (a) or (b) of this paragraph.

In particular, it was observed that in plants, especially in Arabidopsis thaliana, increasing or generating the activity of a gene product non-targeted with the activity of a “b3151-protein”, preferably being encoded by a gene comprising the nucleic acid sequence SEQ ID NO. 42477, preferably the coding region thereof, conferred the production of or the increase in myo-inositol compared with the wild type control.

The nucleic acid sequence of B3151 from Escherichia coli, e.g. as shown in the respective line in column 5 of Table I, application no. 17, has been published in B. And the activity of the gene product thereof is the activity of b3151-protein.

Accordingly, in one embodiment, the process of the present invention for producing raffinose in a non-human organism, like a microorganism or a plant or a part thereof, comprises increasing or generating the activity of a gene product with the activity of a gene product conferring the activity of “b3151-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said B3151, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3151, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3151, or a functional equivalent or a homolog thereof as depicted in column 8 of Table II, application no. 17, preferably a homolog or functional equivalent as depicted in column 8 of Table II B, application no. 17, and being depicted in the same respective line as said B3151, and preferably the activity is increased non-targeted, whereby the respective line disclose in column 7 the fine chemical raffinose.

Accordingly, in one embodiment, the molecule which activity is to be increased in the process of the invention is the gene product with an activity as a “b3151-protein”, preferably it is the molecule of section (a) or (b) of this paragraph.

In particular, it was observed that in plants, especially in Arabidopsis thaliana, increasing or generating the activity of a gene product non-targeted with the activity of a “b3151-protein”, preferably being encoded by a gene comprising the nucleic acid sequence SEQ ID NO. 42477, preferably the coding region thereof, conferred the production of or the increase in raffinose compared with the wild type control.

The nucleic acid sequence of B3151 from Escherichia coli, e.g. as shown in the respective line in column 5 of Table I, application no. 17, has been published in B. And the activity of the gene product thereof is the activity of b3151-protein.

Accordingly, in one embodiment, the process of the present invention for producing sucrose in a non-human organism, like a microorganism or a plant or a part thereof, comprises increasing or generating the activity of a gene product with the activity of a gene product conferring the activity of “b3151-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said B3151, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3151, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3151, or a functional equivalent or a homolog thereof as depicted in column 8 of Table II, application no. 17, preferably a homolog or functional equivalent as depicted in column 8 of Table II B, application no. 17, and being depicted in the same respective line as said B3151, and preferably the activity is increased non-targeted, whereby the respective line disclose in column 7 the fine chemical sucrose.

Accordingly, in one embodiment, the molecule which activity is to be increased in the process of the invention is the gene product with an activity as a “b3151-protein”, preferably it is the molecule of section (a) or (b) of this paragraph.

In particular, it was observed that in plants, especially in Arabidopsis thaliana, increasing or generating the activity of a gene product non-targeted with the activity of a “b3151-protein”, preferably being encoded by a gene comprising the nucleic acid sequence SEQ ID NO. 42477, preferably the coding region thereof, conferred the production of or the increase in sucrose compared with the wild type control.

The nucleic acid sequence of B3221 from Escherichia coli, e.g. as shown in the respective line in column 5 of Table I, application no. 17, has been published in B. And the activity of the gene product thereof is the activity of b3221-protein.

Accordingly, in one embodiment, the process of the present invention for producing fructose in a non-human organism, like a microorganism or a plant or a part thereof, comprises increasing or generating the activity of a gene product with the activity of a gene product conferring the activity of “b3221-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B3221, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3221, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3221, or a functional equivalent or a homolog thereof as depicted in column 8 of Table II, application no. 17, preferably a homolog or functional equivalent as depicted in column 8 of Table II B, application no. 17, and being depicted in the same respective line as said B3221, and preferably the activity is increased plastidic, whereby the respective line disclose in column 7 the fine chemical fructose.

Accordingly, in one embodiment, the molecule which activity is to be increased in the process of the invention is the gene product with an activity as a “b3221-protein”, preferably it is the molecule of section (a) or (b) of this paragraph.

In particular, it was observed that in plants, especially in Arabidopsis thaliana, increasing or generating the activity of a gene product plastidic with the activity of a “b3221-protein”, preferably being encoded by a gene comprising the nucleic acid sequence SEQ ID NO. 82510, preferably the coding region thereof, conferred the production of or the increase in fructose compared with the wild type control.

The nucleic acid sequence of B3221 from Escherichia coli, e.g. as shown in the respective line in column 5 of Table I, application no. 17, has been published in B. And the activity of the gene product thereof is the activity of b3221-protein.

Accordingly, in one embodiment, the process of the present invention for producing glucose in a non-human organism, like a microorganism or a plant or a part thereof, comprises increasing or generating the activity of a gene product with the activity of a gene product conferring the activity of “b3221-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B3221, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3221, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3221, or a functional equivalent or a homolog thereof as depicted in column 8 of Table II, application no. 17, preferably a homolog or functional equivalent as depicted in column 8 of Table II B, application no. 17, and being depicted in the same respective line as said B3221, and preferably the activity is increased plastidic, whereby the respective line disclose in column 7 the fine chemical glucose.

Accordingly, in one embodiment, the molecule which activity is to be increased in the process of the invention is the gene product with an activity as a “b3221-protein”, preferably it is the molecule of section (a) or (b) of this paragraph.

In particular, it was observed that in plants, especially in Arabidopsis thaliana, increasing or generating the activity of a gene product plastidic with the activity of a “b3221-protein”, preferably being encoded by a gene comprising the nucleic acid sequence SEQ ID NO. 82510, preferably the coding region thereof, conferred the production of or the increase in glucose compared with the wild type control.

The nucleic acid sequence of B3221 from Escherichia coli, e.g. as shown in the respective line in column 5 of Table I, application no. 17, has been published in B. And the activity of the gene product thereof is the activity of b3221-protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “b3221-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B3221, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3221, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3221, or a functional equivalent or a homolog thereof as depicted in column 8 of Table II, application no. 17, preferably a homolog or functional equivalent as depicted in column 8 of Table II B, application no. 17, and being depicted in the same respective line as said B3221, and preferably the activity is increased plastidic, whereby the respective line disclose in column 7 the fine chemical 3,4,5-trihydroxypentanoic acid.

Accordingly, in one embodiment, the molecule which activity is to be increased in the process of the invention is the gene product with an activity as a “b3221-protein”, preferably it is the molecule of section (a) or (b) of this paragraph.

In particular, it was observed that in plants, especially in Arabidopsis thaliana, increasing or generating the activity of a gene product plastidic with the activity of a “b3221-protein”, preferably being encoded by a gene comprising the nucleic acid sequence SEQ ID NO. 82510, preferably the coding region thereof, conferred the production of or the increase in 3,4,5-trihydroxypentanoic acid compared with the wild type control.

The nucleic acid sequence of B3246 from Escherichia coli, e.g. as shown in the respective line in column 5 of Table I, application no. 17, has been published in B. And the activity of the gene product thereof is the activity of b3246-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “b3246-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B3246, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3246, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3246, or a functional equivalent or a homolog thereof as depicted in column 8 of Table II, application no. 17, preferably a homolog or functional equivalent as depicted in column 8 of Table II B, application no. 17, and being depicted in the same respective line as said B3246, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “b3246-protein”, preferably it is the molecule of section (a) or (b) of this paragraph.

In particular, it was observed that in plants, especially in Arabidopsis thaliana, increasing or generating the activity of a gene product non-targeted with the activity of a “b3246-protein”, preferably being encoded by a gene comprising the nucleic acid sequence SEQ ID NO. 9470, preferably the coding region thereof, conferred the production of or the increase in myo-inositol compared with the wild type control.

The nucleic acid sequence of B3256 from Escherichia coli, e.g. as shown in the respective line in column 5 of Table I, application no. 17, has been published in B. And the activity of the gene product thereof is the activity of acetyl CoA carboxylase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “acetyl CoA carboxylase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B3256, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3256, or a functional equivalent or a homolog thereof as depicted in column 8 of Table II, application no. 17, preferably a homolog or functional equivalent as depicted in column 8 of Table II B, application no. 17, and being depicted in the same respective line as said B3256, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “acetyl CoA carboxylase”, preferably it is the molecule of section (a) or (b) of this paragraph.

In particular, it was observed that in plants, especially in Arabidopsis thaliana, increasing or generating the activity of a gene product non-targeted with the activity of a “acetyl CoA carboxylase”, preferably being encoded by a gene comprising the nucleic acid sequence SEQ ID NO. 9492, preferably the coding region thereof, conferred the production of or the increase in myo-inositol compared with the wild type control.

The nucleic acid sequence of B3262 from Escherichia coli, e.g. as shown in the respective line in column 5 of Table I, application no. 17, has been published in B. And the activity of the gene product thereof is the activity of methyltransferase.

Accordingly, in one embodiment, the process of the present invention for producing fructose in a non-human organism, like a microorganism or a plant or a part thereof, comprises increasing or generating the activity of a gene product with the activity of a gene product conferring the activity of “methyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B3262, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3262, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3262, or a functional equivalent or a homolog thereof as depicted in column 8 of Table II, application no. 17, preferably a homolog or functional equivalent as depicted in column 8 of Table II B, application no. 17, and being depicted in the same respective line as said B3262, and preferably the activity is increased non-targeted, whereby the respective line disclose in column 7 the fine chemical fructose.

Accordingly, in one embodiment, the molecule which activity is to be increased in the process of the invention is the gene product with an activity as a “methyltransferase”, preferably it is the molecule of section (a) or (b) of this paragraph.

In particular, it was observed that in plants, especially in Arabidopsis thaliana, increasing or generating the activity of a gene product non-targeted with the activity of a “methyltransferase”, preferably being encoded by a gene comprising the nucleic acid sequence SEQ ID NO. 42502, preferably the coding region thereof, conferred the production of or the increase in fructose compared with the wild type control.

The nucleic acid sequence of B3262 from Escherichia coli, e.g. as shown in the respective line in column 5 of Table I, application no. 17, has been published in B. And the activity of the gene product thereof is the activity of methyltransferase.

Accordingly, in one embodiment, the process of the present invention for producing glucose in a non-human organism, like a microorganism or a plant or a part thereof, comprises increasing or generating the activity of a gene product with the activity of a gene product conferring the activity of “methyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B3262, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3262, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3262, or a functional equivalent or a homolog thereof as depicted in column 8 of Table II, application no. 17, preferably a homolog or functional equivalent as depicted in column 8 of Table II B, application no. 17, and being depicted in the same respective line as said B3262, and preferably the activity is increased non-targeted, whereby the respective line disclose in column 7 the fine chemical glucose.

Accordingly, in one embodiment, the molecule which activity is to be increased in the process of the invention is the gene product with an activity as a “methyltransferase”, preferably it is the molecule of section (a) or (b) of this paragraph.

In particular, it was observed that in plants, especially in Arabidopsis thaliana, increasing or generating the activity of a gene product non-targeted with the activity of a “methyltransferase”, preferably being encoded by a gene comprising the nucleic acid sequence SEQ ID NO. 42502, preferably the coding region thereof, conferred the production of or the increase in glucose compared with the wild type control.

The nucleic acid sequence of B3262 from Escherichia coli, e.g. as shown in the respective line in column 5 of Table I, application no. 17, has been published in B. And the activity of the gene product thereof is the activity of methyltransferase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “methyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B3262, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3262, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3262, or a functional equivalent or a homolog thereof as depicted in column 8 of Table II, application no. 17, preferably a homolog or functional equivalent as depicted in column 8 of Table II B, application no. 17, and being depicted in the same respective line as said B3262, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “methyltransferase”, preferably it is the molecule of section (a) or (b) of this paragraph.

In particular, it was observed that in plants, especially in Arabidopsis thaliana, increasing or generating the activity of a gene product non-targeted with the activity of a “methyltransferase”, preferably being encoded by a gene comprising the nucleic acid sequence SEQ ID NO. 42502, preferably the coding region thereof, conferred the production of or the increase in glucose-1-phosphate compared with the wild type control.

The nucleic acid sequence of B3262 from Escherichia coli, e.g. as shown in the respective line in column 5 of Table I, application no. 17, has been published in B. And the activity of the gene product thereof is the activity of methyltransferase.

Accordingly, in one embodiment, the process of the present invention for producing raffinose in a non-human organism, like a microorganism or a plant or a part thereof, comprises increasing or generating the activity of a gene product with the activity of a gene product conferring the activity of “methyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said B3262, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3262, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3262, or a functional equivalent or a homolog thereof as depicted in column 8 of Table II, application no. 17, preferably a homolog or functional equivalent as depicted in column 8 of Table II B, application no. 17, and being depicted in the same respective line as said B3262, and preferably the activity is increased non-targeted, whereby the respective line disclose in column 7 the fine chemical raffinose.

Accordingly, in one embodiment, the molecule which activity is to be increased in the process of the invention is the gene product with an activity as a “methyltransferase”, preferably it is the molecule of section (a) or (b) of this paragraph.

In particular, it was observed that in plants, especially in Arabidopsis thaliana, increasing or generating the activity of a gene product non-targeted with the activity of a “methyltransferase”, preferably being encoded by a gene comprising the nucleic acid sequence SEQ ID NO. 42502, preferably the coding region thereof, conferred the production of or the increase in raffinose compared with the wild type control.

The nucleic acid sequence of B3262 from Escherichia coli, e.g. as shown in the respective line in column 5 of Table I, application no. 17, has been published in B. And the activity of the gene product thereof is the activity of methyltransferase.

Accordingly, in one embodiment, the process of the present invention for producing sucrose in a non-human organism, like a microorganism or a plant or a part thereof, comprises increasing or generating the activity of a gene product with the activity of a gene product conferring the activity of “methyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said B3262, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3262, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3262, or a functional equivalent or a homolog thereof as depicted in column 8 of Table II, application no. 17, preferably a homolog or functional equivalent as depicted in column 8 of Table II B, application no. 17, and being depicted in the same respective line as said B3262, and preferably the activity is increased non-targeted, whereby the respective line disclose in column 7 the fine chemical sucrose.

Accordingly, in one embodiment, the molecule which activity is to be increased in the process of the invention is the gene product with an activity as a “methyltransferase”, preferably it is the molecule of section (a) or (b) of this paragraph.

In particular, it was observed that in plants, especially in Arabidopsis thaliana, increasing or generating the activity of a gene product non-targeted with the activity of a “methyltransferase”, preferably being encoded by a gene comprising the nucleic acid sequence SEQ ID NO. 42502, preferably the coding region thereof, conferred the production of or the increase in sucrose compared with the wild type control.

The nucleic acid sequence of B3337 from Escherichia coli, e.g. as shown in the respective line in column 5 of Table I, application no. 17, has been published in B. And the activity of the gene product thereof is the activity of ferredoxin.

Accordingly, in one embodiment, the process of the present invention for producing glucose in a non-human organism, like a microorganism or a plant or a part thereof, comprises increasing or generating the activity of a gene product with the activity of a gene product conferring the activity of “ferredoxin”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B3337, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3337, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3337, or a functional equivalent or a homolog thereof as depicted in column 8 of Table II, application no. 17, preferably a homolog or functional equivalent as depicted in column 8 of Table II B, application no. 17, and being depicted in the same respective line as said B3337, and preferably the activity is increased plastidic, whereby the respective line disclose in column 7 the fine chemical glucose.

Accordingly, in one embodiment, the molecule which activity is to be increased in the process of the invention is the gene product with an activity as a “ferredoxin”, preferably it is the molecule of section (a) or (b) of this paragraph.

In particular, it was observed that in plants, especially in Arabidopsis thaliana, increasing or generating the activity of a gene product plastidic with the activity of a “ferredoxin”, preferably being encoded by a gene comprising the nucleic acid sequence SEQ ID NO. 146364, preferably the coding region thereof, conferred the production of or the increase in glucose compared with the wild type control.

The nucleic acid sequence of B3346 from Escherichia coli, e.g. as shown in the respective line in column 5 of Table I, application no. 17, has been published in B. And the activity of the gene product thereof is the activity of b3346-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “b3346-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B3346, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3346, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3346, or a functional equivalent or a homolog thereof as depicted in column 8 of Table II, application no. 17, preferably a homolog or functional equivalent as depicted in column 8 of Table II B, application no. 17, and being depicted in the same respective line as said B3346, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “b3346-protein”, preferably it is the molecule of section (a) or (b) of this paragraph.

In particular, it was observed that in plants, especially in Arabidopsis thaliana, increasing or generating the activity of a gene product non-targeted with the activity of a “b3346-protein”, preferably being encoded by a gene comprising the nucleic acid sequence SEQ ID NO. 10104, preferably the coding region thereof, conferred the production of or the increase in myo-inositol compared with the wild type control.

The nucleic acid sequence of B3346 from Escherichia coli, e.g. as shown in the respective line in column 5 of Table I, application no. 17, has been published in B. And the activity of the gene product thereof is the activity of b3346-protein.

Accordingly, in one embodiment, the process of the present invention for producing raffinose in a non-human organism, like a microorganism or a plant or a part thereof, comprises increasing or generating the activity of a gene product with the activity of a gene product conferring the activity of “b3346-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said B3346, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3346, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3346, or a functional equivalent or a homolog thereof as depicted in column 8 of Table II, application no. 17, preferably a homolog or functional equivalent as depicted in column 8 of Table II B, application no. 17, and being depicted in the same respective line as said B3346, and preferably the activity is increased non-targeted, whereby the respective line disclose in column 7 the fine chemical raffinose.

Accordingly, in one embodiment, the molecule which activity is to be increased in the process of the invention is the gene product with an activity as a “b3346-protein”, preferably it is the molecule of section (a) or (b) of this paragraph.

In particular, it was observed that in plants, especially in Arabidopsis thaliana, increasing or generating the activity of a gene product non-targeted with the activity of a “b3346-protein”, preferably being encoded by a gene comprising the nucleic acid sequence SEQ ID NO. 10104, preferably the coding region thereof, conferred the production of or the increase in raffinose compared with the wild type control.

The nucleic acid sequence of B3346 from Escherichia coli, e.g. as shown in the respective line in column 5 of Table I, application no. 17, has been published in B. And the activity of the gene product thereof is the activity of b3346-protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “b3346-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B3346, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3346, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3346, or a functional equivalent or a homolog thereof as depicted in column 8 of Table II, application no. 17, preferably a homolog or functional equivalent as depicted in column 8 of Table II B, application no. 17, and being depicted in the same respective line as said B3346, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “b3346-protein”, preferably it is the molecule of section (a) or (b) of this paragraph.

In particular, it was observed that in plants, especially in Arabidopsis thaliana, increasing or generating the activity of a gene product non-targeted with the activity of a “b3346-protein”, preferably being encoded by a gene comprising the nucleic acid sequence SEQ ID NO. 10104, preferably the coding region thereof, conferred the production of or the increase in 3,4,5-trihydroxypentanoic acid compared with the wild type control.

The nucleic acid sequence of B3403 from Escherichia coli, e.g. as shown in the respective line in column 5 of Table I, application no. 17, has been published in B. And the activity of the gene product thereof is the activity of phosphoenolpyruvate carboxykinase.

Accordingly, in one embodiment, the process of the present invention for producing fructose in a non-human organism, like a microorganism or a plant or a part thereof, comprises increasing or generating the activity of a gene product with the activity of a gene product conferring the activity of “phosphoenolpyruvate carboxykinase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B3403, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3403, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3403, or a functional equivalent or a homolog thereof as depicted in column 8 of Table II, application no. 17, preferably a homolog or functional equivalent as depicted in column 8 of Table II B, application no. 17, and being depicted in the same respective line as said B3403, and preferably the activity is increased non-targeted, whereby the respective line disclose in column 7 the fine chemical fructose.

Accordingly, in one embodiment, the molecule which activity is to be increased in the process of the invention is the gene product with an activity as a “phosphoenolpyruvate carboxykinase”, preferably it is the molecule of section (a) or (b) of this paragraph.

In particular, it was observed that in plants, especially in Arabidopsis thaliana, increasing or generating the activity of a gene product non-targeted with the activity of a “phosphoenolpyruvate carboxykinase”, preferably being encoded by a gene comprising the nucleic acid sequence SEQ ID NO. 82866, preferably the coding region thereof, conferred the production of or the increase in fructose compared with the wild type control.

The nucleic acid sequence of B3403 from Escherichia coli, e.g. as shown in the respective line in column 5 of Table I, application no. 17, has been published in B. And the activity of the gene product thereof is the activity of phosphoenolpyruvate carboxykinase.

Accordingly, in one embodiment, the process of the present invention for producing glucose in a non-human organism, like a microorganism or a plant or a part thereof, comprises increasing or generating the activity of a gene product with the activity of a gene product conferring the activity of “phosphoenolpyruvate carboxykinase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B3403, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3403, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3403, or a functional equivalent or a homolog thereof as depicted in column 8 of Table II, application no. 17, preferably a homolog or functional equivalent as depicted in column 8 of Table II B, application no. 17, and being depicted in the same respective line as said B3403, and preferably the activity is increased non-targeted, whereby the respective line disclose in column 7 the fine chemical glucose.

Accordingly, in one embodiment, the molecule which activity is to be increased in the process of the invention is the gene product with an activity as a “phosphoenolpyruvate carboxykinase”, preferably it is the molecule of section (a) or (b) of this paragraph.

In particular, it was observed that in plants, especially in Arabidopsis thaliana, increasing or generating the activity of a gene product non-targeted with the activity of a “phosphoenolpyruvate carboxykinase”, preferably being encoded by a gene comprising the nucleic acid sequence SEQ ID NO. 82866, preferably the coding region thereof, conferred the production of or the increase in glucose compared with the wild type control.

The nucleic acid sequence of B3427 from Escherichia coli, e.g. as shown in the respective line in column 5 of Table I, application no. 17, has been published in B. And the activity of the gene product thereof is the activity of B3427-protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “B3427-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B3427, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3427, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3427, or a functional equivalent or a homolog thereof as depicted in column 8 of Table II, application no. 17, preferably a homolog or functional equivalent as depicted in column 8 of Table II B, application no. 17, and being depicted in the same respective line as said B3427, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “B3427-protein”, preferably it is the molecule of section (a) or (b) of this paragraph.

In particular, it was observed that in plants, especially in Arabidopsis thaliana, increasing or generating the activity of a gene product non-targeted with the activity of a “B3427-protein”, preferably being encoded by a gene comprising the nucleic acid sequence SEQ ID NO. 42579, preferably the coding region thereof, conferred the production of or the increase in 3,4,5-trihydroxypentanoic acid compared with the wild type control.

The nucleic acid sequence of B3429 from Escherichia coli, e.g. as shown in the respective line in column 5 of Table I, application no. 17, has been published in B. And the activity of the gene product thereof is the activity of glycogen synthase.

Accordingly, in one embodiment, the process of the present invention for producing fructose in a non-human organism, like a microorganism or a plant or a part thereof, comprises increasing or generating the activity of a gene product with the activity of a gene product conferring the activity of “glycogen synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B3429, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3429, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3429, or a functional equivalent or a homolog thereof as depicted in column 8 of Table II, application no. 17, preferably a homolog or functional equivalent as depicted in column 8 of Table II B, application no. 17, and being depicted in the same respective line as said B3429, and preferably the activity is increased mitochondrial, whereby the respective line disclose in column 7 the fine chemical fructose.

Accordingly, in one embodiment, the molecule which activity is to be increased in the process of the invention is the gene product with an activity as a “glycogen synthase”, preferably it is the molecule of section (a) or (b) of this paragraph.

In particular, it was observed that in plants, especially in Arabidopsis thaliana, increasing or generating the activity of a gene product mitochondrial with the activity of a “glycogen synthase”, preferably being encoded by a gene comprising the nucleic acid sequence SEQ ID NO. 94051, preferably the coding region thereof, conferred the production of or the increase in fructose compared with the wild type control.

The nucleic acid sequence of B3429 from Escherichia coli, e.g. as shown in the respective line in column 5 of Table I, application no. 17, has been published in B. And the activity of the gene product thereof is the activity of glycogen synthase.

Accordingly, in one embodiment, the process of the present invention for producing glucose in a non-human organism, like a microorganism or a plant or a part thereof, comprises increasing or generating the activity of a gene product with the activity of a gene product conferring the activity of “glycogen synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B3429, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3429, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3429, or a functional equivalent or a homolog thereof as depicted in column 8 of Table II, application no. 17, preferably a homolog or functional equivalent as depicted in column 8 of Table II B, application no. 17, and being depicted in the same respective line as said B3429, and preferably the activity is increased mitochondrial, whereby the respective line disclose in column 7 the fine chemical glucose.

Accordingly, in one embodiment, the molecule which activity is to be increased in the process of the invention is the gene product with an activity as a “glycogen synthase”, preferably it is the molecule of section (a) or (b) of this paragraph.

In particular, it was observed that in plants, especially in Arabidopsis thaliana, increasing or generating the activity of a gene product mitochondrial with the activity of a “glycogen synthase”, preferably being encoded by a gene comprising the nucleic acid sequence SEQ ID NO. 94051, preferably the coding region thereof, conferred the production of or the increase in glucose compared with the wild type control.

The nucleic acid sequence of B3442 from Escherichia coli, e.g. as shown in the respective line in column 5 of Table I, application no. 17, has been published in B. And the activity of the gene product thereof is the activity of b3442-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “b3442-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B3442, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3442, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3442, or a functional equivalent or a homolog thereof as depicted in column 8 of Table II, application no. 17, preferably a homolog or functional equivalent as depicted in column 8 of Table II B, application no. 17, and being depicted in the same respective line as said B3442, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “b3442-protein”, preferably it is the molecule of section (a) or (b) of this paragraph.

In particular, it was observed that in plants, especially in Arabidopsis thaliana, increasing or generating the activity of a gene product non-targeted with the activity of a “b3442-protein”, preferably being encoded by a gene comprising the nucleic acid sequence SEQ ID NO. 94380, preferably the coding region thereof, conferred the production of or the increase in myo-inositol compared with the wild type control.

The nucleic acid sequence of B3443 from Escherichia coli, e.g. as shown in the respective line in column 5 of Table I, application no. 17, has been published in B. And the activity of the gene product thereof is the activity of B3443-protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “B3443-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B3443, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3443, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3443, or a functional equivalent or a homolog thereof as depicted in column 8 of Table II, application no. 17, preferably a homolog or functional equivalent as depicted in column 8 of Table II B, application no. 17, and being depicted in the same respective line as said B3443, and preferably the activity is increased plastidic, whereby the respective line disclose in column 7 the fine chemical 3,4,5-trihydroxypentanoic acid.

Accordingly, in one embodiment, the molecule which activity is to be increased in the process of the invention is the gene product with an activity as a “B3443-protein”, preferably it is the molecule of section (a) or (b) of this paragraph.

In particular, it was observed that in plants, especially in Arabidopsis thaliana, increasing or generating the activity of a gene product plastidic with the activity of a “B3443-protein”, preferably being encoded by a gene comprising the nucleic acid sequence SEQ ID NO. 146440, preferably the coding region thereof, conferred the production of or the increase in 3,4,5-trihydroxypentanoic acid compared with the wild type control.

The nucleic acid sequence of B3451 from Escherichia coli, e.g. as shown in the respective line in column 5 of Table I, application no. 17, has been published in B. And the activity of the gene product thereof is the activity of sn-glycerol-3-phosphate transport system permease protein.

Accordingly, in one embodiment, the process of the present invention for producing saccharic acid in a non-human organism, like a microorganism or a plant or a part thereof, comprises increasing or generating the activity of a gene product with the activity of a gene product conferring the activity of “sn-glycerol-3-phosphate transport system permease protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical saccharic acid), application no. 17, preferably the coding region thereof, or a homolog or a fragment thereof, and being depicted in the same respective line as said B3451, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3451, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3451, or a functional equivalent or a homolog thereof as depicted in column 8 of Table II, application no. 17, preferably a homolog or functional equivalent as depicted in column 8 of Table II B, application no. 17, and being depicted in the same respective line as said B3451, and preferably the activity is increased plastidic, whereby the respective line disclose in column 7 the fine chemical saccharic acid.

Accordingly, in one embodiment, the molecule which activity is to be increased in the process of the invention is the gene product with an activity as a “sn-glycerol-3-phosphate transport system permease protein”, preferably it is the molecule of section (a) or (b) of this paragraph.

In particular, it was observed that in plants, especially in Arabidopsis thaliana, increasing or generating the activity of a gene product plastidic with the activity of a “sn-glycerol-3-phosphate transport system permease protein”, preferably being encoded by a gene comprising the nucleic acid sequence SEQ ID NO. 116172, preferably the coding region thereof, conferred the production of or the increase in saccharic acid compared with the wild type control.

The nucleic acid sequence of B3457 from Escherichia coli, e.g. as shown in the respective line in column 5 of Table I, application no. 17, has been published in B. And the activity of the gene product thereof is the activity of high-affinity branched-chain amino acid transport protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “high-affinity branched-chain amino acid transport protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B3457, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3457, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3457, or a functional equivalent or a homolog thereof as depicted in column 8 of Table II, application no. 17, preferably a homolog or functional equivalent as depicted in column 8 of Table II B, application no. 17, and being depicted in the same respective line as said B3457, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “high-affinity branched-chain amino acid transport protein”, preferably it is the molecule of section (a) or (b) of this paragraph.

In particular, it was observed that in plants, especially in Arabidopsis thaliana, increasing or generating the activity of a gene product non-targeted with the activity of a “high-affinity branched-chain amino acid transport protein”, preferably being encoded by a gene comprising the nucleic acid sequence SEQ ID NO. 146472, preferably the coding region thereof, conferred the production of or the increase in glucose-1-phosphate compared with the wild type control.

The nucleic acid sequence of B3457 from Escherichia coli, e.g. as shown in the respective line in column 5 of Table I, application no. 17, has been published in B. And the activity of the gene product thereof is the activity of high-affinity branched-chain amino acid transport protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “high-affinity branched-chain amino acid transport protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B3457, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3457, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3457, or a functional equivalent or a homolog thereof as depicted in column 8 of Table II, application no. 17, preferably a homolog or functional equivalent as depicted in column 8 of Table II B, application no. 17, and being depicted in the same respective line as said B3457, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “high-affinity branched-chain amino acid transport protein”, preferably it is the molecule of section (a) or (b) of this paragraph.

In particular, it was observed that in plants, especially in Arabidopsis thaliana, increasing or generating the activity of a gene product non-targeted with the activity of a “high-affinity branched-chain amino acid transport protein”, preferably being encoded by a gene comprising the nucleic acid sequence SEQ ID NO. 146472, preferably the coding region thereof, conferred the production of or the increase in myo-inositol compared with the wild type control.

The nucleic acid sequence of B3519 from Escherichia coli, e.g. as shown in the respective line in column 5 of Table I, application no. 17, has been published in B. And the activity of the gene product thereof is the activity of trehalase.

Accordingly, in one embodiment, the process of the present invention for producing fructose in a non-human organism, like a microorganism or a plant or a part thereof, comprises increasing or generating the activity of a gene product with the activity of a gene product conferring the activity of “trehalase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B3519, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3519, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3519, or a functional equivalent or a homolog thereof as depicted in column 8 of Table II, application no. 17, preferably a homolog or functional equivalent as depicted in column 8 of Table II B, application no. 17, and being depicted in the same respective line as said B3519, and preferably the activity is increased plastidic, whereby the respective line disclose in column 7 the fine chemical fructose.

Accordingly, in one embodiment, the molecule which activity is to be increased in the process of the invention is the gene product with an activity as a “trehalase”, preferably it is the molecule of section (a) or (b) of this paragraph.

In particular, it was observed that in plants, especially in Arabidopsis thaliana, increasing or generating the activity of a gene product plastidic with the activity of a “trehalase”, preferably being encoded by a gene comprising the nucleic acid sequence SEQ ID NO. 146646, preferably the coding region thereof, conferred the production of or the increase in fructose compared with the wild type control.

The nucleic acid sequence of B3519 from Escherichia coli, e.g. as shown in the respective line in column 5 of Table I, application no. 17, has been published in B. And the activity of the gene product thereof is the activity of trehalase.

Accordingly, in one embodiment, the process of the present invention for producing galactinol in a non-human organism, like a microorganism or a plant or a part thereof, comprises increasing or generating the activity of a gene product with the activity of a gene product conferring the activity of “trehalase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical galactinol), application no. 17, preferably the coding region thereof, or a homolog or a fragment thereof, and being depicted in the same respective line as said B3519, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3519, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3519, or a functional equivalent or a homolog thereof as depicted in column 8 of Table II, application no. 17, preferably a homolog or functional equivalent as depicted in column 8 of Table II B, application no. 17, and being depicted in the same respective line as said B3519, and preferably the activity is increased plastidic, whereby the respective line disclose in column 7 the fine chemical galactinol.

Accordingly, in one embodiment, the molecule which activity is to be increased in the process of the invention is the gene product with an activity as a “trehalase”, preferably it is the molecule of section (a) or (b) of this paragraph.

In particular, it was observed that in plants, especially in Arabidopsis thaliana, increasing or generating the activity of a gene product plastidic with the activity of a “trehalase”, preferably being encoded by a gene comprising the nucleic acid sequence SEQ ID NO. 146646, preferably the coding region thereof, conferred the production of or the increase in galactinol compared with the wild type control.

The nucleic acid sequence of B3600 from Escherichia coli, e.g. as shown in the respective line in column 5 of Table I, application no. 17, has been published in B. And the activity of the gene product thereof is the activity of mannitol-1-phosphate dehydrogenase.

Accordingly, in one embodiment, the process of the present invention for producing fructose in a non-human organism, like a microorganism or a plant or a part thereof, comprises increasing or generating the activity of a gene product with the activity of a gene product conferring the activity of “mannitol-1-phosphate dehydrogenase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B3600, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3600, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3600, or a functional equivalent or a homolog thereof as depicted in column 8 of Table II, application no. 17, preferably a homolog or functional equivalent as depicted in column 8 of Table II B, application no. 17, and being depicted in the same respective line as said B3600, and preferably the activity is increased non-targeted, whereby the respective line disclose in column 7 the fine chemical fructose.

Accordingly, in one embodiment, the molecule which activity is to be increased in the process of the invention is the gene product with an activity as a “mannitol-1-phosphate dehydrogenase”, preferably it is the molecule of section (a) or (b) of this paragraph.

In particular, it was observed that in plants, especially in Arabidopsis thaliana, increasing or generating the activity of a gene product non-targeted with the activity of a “mannitol-1-phosphate dehydrogenase”, preferably being encoded by a gene comprising the nucleic acid sequence SEQ ID NO. 146732, preferably the coding region thereof, conferred the production of or the increase in fructose compared with the wild type control.

The nucleic acid sequence of B3600 from Escherichia coli, e.g. as shown in the respective line in column 5 of Table I, application no. 17, has been published in B. And the activity of the gene product thereof is the activity of mannitol-1-phosphate dehydrogenase.

Accordingly, in one embodiment, the process of the present invention for producing mannitol in a non-human organism, like a microorganism or a plant or a part thereof, comprises increasing or generating the activity of a gene product with the activity of a gene product conferring the activity of “mannitol-1-phosphate dehydrogenase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical mannitol), application no. 17, preferably the coding region thereof, or a homolog or a fragment thereof, and being depicted in the same respective line as said B3600, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3600, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3600, or a functional equivalent or a homolog thereof as depicted in column 8 of Table II, application no. 17, preferably a homolog or functional equivalent as depicted in column 8 of Table II B, application no. 17, and being depicted in the same respective line as said B3600, and preferably the activity is increased non-targeted, whereby the respective line disclose in column 7 the fine chemical mannitol.

Accordingly, in one embodiment, the molecule which activity is to be increased in the process of the invention is the gene product with an activity as a “mannitol-1-phosphate dehydrogenase”, preferably it is the molecule of section (a) or (b) of this paragraph.

In particular, it was observed that in plants, especially in Arabidopsis thaliana, increasing or generating the activity of a gene product non-targeted with the activity of a “mannitol-1-phosphate dehydrogenase”, preferably being encoded by a gene comprising the nucleic acid sequence SEQ ID NO. 146732, preferably the coding region thereof, conferred the production of or the increase in mannitol compared with the wild type control.

The nucleic acid sequence of B3600 from Escherichia coli, e.g. as shown in the respective line in column 5 of Table I, application no. 17, has been published in B. And the activity of the gene product thereof is the activity of mannitol-1-phosphate dehydrogenase.

Accordingly, in one embodiment, the process of the present invention for producing sorbitol in a non-human organism, like a microorganism or a plant or a part thereof, comprises increasing or generating the activity of a gene product with the activity of a gene product conferring the activity of “mannitol-1-phosphate dehydrogenase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical sorbitol), application no. 17, preferably the coding region thereof, or a homolog or a fragment thereof, and being depicted in the same respective line as said B3600, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3600, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3600, or a functional equivalent or a homolog thereof as depicted in column 8 of Table II, application no. 17, preferably a homolog or functional equivalent as depicted in column 8 of Table II B, application no. 17, and being depicted in the same respective line as said B3600, and preferably the activity is increased non-targeted, whereby the respective line disclose in column 7 the fine chemical sorbitol.

Accordingly, in one embodiment, the molecule which activity is to be increased in the process of the invention is the gene product with an activity as a “mannitol-1-phosphate dehydrogenase”, preferably it is the molecule of section (a) or (b) of this paragraph.

In particular, it was observed that in plants, especially in Arabidopsis thaliana, increasing or generating the activity of a gene product non-targeted with the activity of a “mannitol-1-phosphate dehydrogenase”, preferably being encoded by a gene comprising the nucleic acid sequence SEQ ID NO. 146732, preferably the coding region thereof, conferred the production of or the increase in sorbitol compared with the wild type control.

The nucleic acid sequence of B3774 from Escherichia coli, e.g. as shown in the respective line in column 5 of Table I, application no. 17, has been published in B. And the activity of the gene product thereof is the activity of ketol-acid reductoisomerase.

Accordingly, in one embodiment, the process of the present invention for producing galactinol in a non-human organism, like a microorganism or a plant or a part thereof, comprises increasing or generating the activity of a gene product with the activity of a gene product conferring the activity of “ketol-acid reductoisomerase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical galactinol), application no. 17, preferably the coding region thereof, or a homolog or a fragment thereof, and being depicted in the same respective line as said B3774, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3774, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3774, or a functional equivalent or a homolog thereof as depicted in column 8 of Table II, application no. 17, preferably a homolog or functional equivalent as depicted in column 8 of Table II B, application no. 17, and being depicted in the same respective line as said B3774, and preferably the activity is increased non-targeted, whereby the respective line disclose in column 7 the fine chemical galactinol.

Accordingly, in one embodiment, the molecule which activity is to be increased in the process of the invention is the gene product with an activity as a “ketol-acid reductoisomerase”, preferably it is the molecule of section (a) or (b) of this paragraph.

In particular, it was observed that in plants, especially in Arabidopsis thaliana, increasing or generating the activity of a gene product non-targeted with the activity of a “ketol-acid reductoisomerase”, preferably being encoded by a gene comprising the nucleic acid sequence SEQ ID NO. 94386, preferably the coding region thereof, conferred the production of or the increase in galactinol compared with the wild type control.

The nucleic acid sequence of B3777 from Escherichia coli, e.g. as shown in the respective line in column 5 of Table I, application no. 17, has been published in B. And the activity of the gene product thereof is the activity of b3777-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “b3777-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B3777, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3777, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3777, or a functional equivalent or a homolog thereof as depicted in column 8 of Table II, application no. 17, preferably a homolog or functional equivalent as depicted in column 8 of Table II B, application no. 17, and being depicted in the same respective line as said B3777, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “b3777-protein”, preferably it is the molecule of section (a) or (b) of this paragraph.

In particular, it was observed that in plants, especially in Arabidopsis thaliana, increasing or generating the activity of a gene product non-targeted with the activity of a “b3777-protein”, preferably being encoded by a gene comprising the nucleic acid sequence SEQ ID NO. 136098, preferably the coding region thereof, conferred the production of or the increase in myo-inositol compared with the wild type control.

The nucleic acid sequence of B3817 from Escherichia coli, e.g. as shown in the respective line in column 5 of Table I, application no. 17, has been published in B. And the activity of the gene product thereof is the activity of b3817-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “b3817-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B3817, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3817, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3817, or a functional equivalent or a homolog thereof as depicted in column 8 of Table II, application no. 17, preferably a homolog or functional equivalent as depicted in column 8 of Table II B, application no. 17, and being depicted in the same respective line as said B3817, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “b3817-protein”, preferably it is the molecule of section (a) or (b) of this paragraph.

In particular, it was observed that in plants, especially in Arabidopsis thaliana, increasing or generating the activity of a gene product non-targeted with the activity of a “b3817-protein”, preferably being encoded by a gene comprising the nucleic acid sequence SEQ ID NO. 10708, preferably the coding region thereof, conferred the production of or the increase in myo-inositol compared with the wild type control.

The nucleic acid sequence of B3817 from Escherichia coli, e.g. as shown in the respective line in column 5 of Table I, application no. 17, has been published in B. And the activity of the gene product thereof is the activity of b3817-protein.

Accordingly, in one embodiment, the process of the present invention for producing raffinose in a non-human organism, like a microorganism or a plant or a part thereof, comprises increasing or generating the activity of a gene product with the activity of a gene product conferring the activity of “b3817-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said B3817, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3817, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3817, or a functional equivalent or a homolog thereof as depicted in column 8 of Table II, application no. 17, preferably a homolog or functional equivalent as depicted in column 8 of Table II B, application no. 17, and being depicted in the same respective line as said B3817, and preferably the activity is increased non-targeted, whereby the respective line disclose in column 7 the fine chemical raffinose.

Accordingly, in one embodiment, the molecule which activity is to be increased in the process of the invention is the gene product with an activity as a “b3817-protein”, preferably it is the molecule of section (a) or (b) of this paragraph.

In particular, it was observed that in plants, especially in Arabidopsis thaliana, increasing or generating the activity of a gene product non-targeted with the activity of a “b3817-protein”, preferably being encoded by a gene comprising the nucleic acid sequence SEQ ID NO. 10708, preferably the coding region thereof, conferred the production of or the increase in raffinose compared with the wild type control.

The nucleic acid sequence of B3818 from Escherichia coli, e.g. as shown in the respective line in column 5 of Table I, application no. 17, has been published in B. And the activity of the gene product thereof is the activity of b3818-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “b3818-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B3818, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3818, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3818, or a functional equivalent or a homolog thereof as depicted in column 8 of Table II, application no. 17, preferably a homolog or functional equivalent as depicted in column 8 of Table II B, application no. 17, and being depicted in the same respective line as said B3818, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “b3818-protein”, preferably it is the molecule of section (a) or (b) of this paragraph.

In particular, it was observed that in plants, especially in Arabidopsis thaliana, increasing or generating the activity of a gene product non-targeted with the activity of a “b3818-protein”, preferably being encoded by a gene comprising the nucleic acid sequence SEQ ID NO. 146834, preferably the coding region thereof, conferred the production of or the increase in myo-inositol compared with the wild type control.

The nucleic acid sequence of B3823 from Escherichia coli, e.g. as shown in the respective line in column 5 of Table I, application no. 17, has been published in B. And the activity of the gene product thereof is the activity of threonine efflux protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “threonine efflux protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B3823, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3823, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3823, or a functional equivalent or a homolog thereof as depicted in column 8 of Table II, application no. 17, preferably a homolog or functional equivalent as depicted in column 8 of Table II B, application no. 17, and being depicted in the same respective line as said B3823, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “threonine efflux protein”, preferably it is the molecule of section (a) or (b) of this paragraph.

In particular, it was observed that in plants, especially in Arabidopsis thaliana, increasing or generating the activity of a gene product non-targeted with the activity of a “threonine efflux protein”, preferably being encoded by a gene comprising the nucleic acid sequence SEQ ID NO. 10726, preferably the coding region thereof, conferred the production of or the increase in myo-inositol compared with the wild type control.

The nucleic acid sequence of B3870 from Escherichia coli, e.g. as shown in the respective line in column 5 of Table I, application no. 17, has been published in B. And the activity of the gene product thereof is the activity of glutamine synthetase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “glutamine synthetase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B3870, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3870, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3870, or a functional equivalent or a homolog thereof as depicted in column 8 of Table II, application no. 17, preferably a homolog or functional equivalent as depicted in column 8 of Table II B, application no. 17, and being depicted in the same respective line as said B3870, and preferably the activity is increased plastidic, whereby the respective line disclose in column 7 the fine chemical myo-inositol.

Accordingly, in one embodiment, the molecule which activity is to be increased in the process of the invention is the gene product with an activity as a “glutamine synthetase”, preferably it is the molecule of section (a) or (b) of this paragraph.

In particular, it was observed that in plants, especially in Arabidopsis thaliana, increasing or generating the activity of a gene product plastidic with the activity of a “glutamine synthetase”, preferably being encoded by a gene comprising the nucleic acid sequence SEQ ID NO. 101327, preferably the coding region thereof, conferred the production of or the increase in myo-inositol compared with the wild type control.

The nucleic acid sequence of B3870 from Escherichia coli, e.g. as shown in the respective line in column 5 of Table I, application no. 17, has been published in B. And the activity of the gene product thereof is the activity of glutamine synthetase.

Accordingly, in one embodiment, the process of the present invention for producing verbascose in a non-human organism, like a microorganism or a plant or a part thereof, comprises increasing or generating the activity of a gene product with the activity of a gene product conferring the activity of “glutamine synthetase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical verbascose), application no. 17, preferably the coding region thereof, or a homolog or a fragment thereof, and being depicted in the same respective line as said B3870, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3870, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3870, or a functional equivalent or a homolog thereof as depicted in column 8 of Table II, application no. 17, preferably a homolog or functional equivalent as depicted in column 8 of Table II B, application no. 17, and being depicted in the same respective line as said B3870, and preferably the activity is increased plastidic, whereby the respective line disclose in column 7 the fine chemical verbascose.

Accordingly, in one embodiment, the molecule which activity is to be increased in the process of the invention is the gene product with an activity as a “glutamine synthetase”, preferably it is the molecule of section (a) or (b) of this paragraph.

In particular, it was observed that in plants, especially in Arabidopsis thaliana, increasing or generating the activity of a gene product plastidic with the activity of a “glutamine synthetase”, preferably being encoded by a gene comprising the nucleic acid sequence SEQ ID NO. 101327, preferably the coding region thereof, conferred the production of or the increase in verbascose compared with the wild type control.

The nucleic acid sequence of B3872 from Escherichia coli, e.g. as shown in the respective line in column 5 of Table I, application no. 17, has been published in B. And the activity of the gene product thereof is the activity of transcriptional regulator.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B3872, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3872, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3872, or a functional equivalent or a homolog thereof as depicted in column 8 of Table II, application no. 17, preferably a homolog or functional equivalent as depicted in column 8 of Table II B, application no. 17, and being depicted in the same respective line as said B3872, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product plastidic with the activity of a “transcriptional regulator”, preferably being encoded by a gene comprising the nucleic acid sequence SEQ ID NO. 83821, preferably the coding region thereof, conferred the production of or the increase in glucose-1-phosphate compared with the wild type control.

The nucleic acid sequence of B3989 from Escherichia coli, e.g. as shown in the respective line in column 5 of Table I, application no. 17, has been published in B. And the activity of the gene product thereof is the activity of b3989-protein.

Accordingly, in one embodiment, the process of the present invention for producing fructose in a non-human organism, like a microorganism or a plant or a part thereof, comprises increasing or generating the activity of a gene product with the activity of a gene product conferring the activity of “b3989-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B3989, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3989, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3989, or a functional equivalent or a homolog thereof as depicted in column 8 of Table II, application no. 17, preferably a homolog or functional equivalent as depicted in column 8 of Table II B, application no. 17, and being depicted in the same respective line as said B3989, and preferably the activity is increased non-targeted, whereby the respective line disclose in column 7 the fine chemical fructose.

Accordingly, in one embodiment, the molecule which activity is to be increased in the process of the invention is the gene product with an activity as a “b3989-protein”, preferably it is the molecule of section (a) or (b) of this paragraph.

In particular, it was observed that in plants, especially in Arabidopsis thaliana, increasing or generating the activity of a gene product non-targeted with the activity of a “b3989-protein”, preferably being encoded by a gene comprising the nucleic acid sequence SEQ ID NO. 44372, preferably the coding region thereof, conferred the production of or the increase in fructose compared with the wild type control.

The nucleic acid sequence of B3989 from Escherichia coli, e.g. as shown in the respective line in column 5 of Table I, application no. 17, has been published in B. And the activity of the gene product thereof is the activity of b3989-protein.

Accordingly, in one embodiment, the process of the present invention for producing glucose in a non-human organism, like a microorganism or a plant or a part thereof, comprises increasing or generating the activity of a gene product with the activity of a gene product conferring the activity of “b3989-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B3989, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3989, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3989, or a functional equivalent or a homolog thereof as depicted in column 8 of Table II, application no. 17, preferably a homolog or functional equivalent as depicted in column 8 of Table II B, application no. 17, and being depicted in the same respective line as said B3989, and preferably the activity is increased non-targeted, whereby the respective line disclose in column 7 the fine chemical glucose.

Accordingly, in one embodiment, the molecule which activity is to be increased in the process of the invention is the gene product with an activity as a “b3989-protein”, preferably it is the molecule of section (a) or (b) of this paragraph.

In particular, it was observed that in plants, especially in Arabidopsis thaliana, increasing or generating the activity of a gene product non-targeted with the activity of a “b3989-protein”, preferably being encoded by a gene comprising the nucleic acid sequence SEQ ID NO. 44372, preferably the coding region thereof, conferred the production of or the increase in glucose compared with the wild type control.

The nucleic acid sequence of B3989 from Escherichia coli, e.g. as shown in the respective line in column 5 of Table I, application no. 17, has been published in B. And the activity of the gene product thereof is the activity of b3989-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “b3989-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B3989, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3989, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3989, or a functional equivalent or a homolog thereof as depicted in column 8 of Table II, application no. 17, preferably a homolog or functional equivalent as depicted in column 8 of Table II B, application no. 17, and being depicted in the same respective line as said B3989, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “b3989-protein”, preferably it is the molecule of section (a) or (b) of this paragraph.

In particular, it was observed that in plants, especially in Arabidopsis thaliana, increasing or generating the activity of a gene product non-targeted with the activity of a “b3989-protein”, preferably being encoded by a gene comprising the nucleic acid sequence SEQ ID NO. 44372, preferably the coding region thereof, conferred the production of or the increase in glucose-1-phosphate compared with the wild type control.

The nucleic acid sequence of B3989 from Escherichia coli, e.g. as shown in the respective line in column 5 of Table I, application no. 17, has been published in B. And the activity of the gene product thereof is the activity of b3989-protein.

Accordingly, in one embodiment, the process of the present invention for producing raffinose in a non-human organism, like a microorganism or a plant or a part thereof, comprises increasing or generating the activity of a gene product with the activity of a gene product conferring the activity of “b3989-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said B3989, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3989, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3989, or a functional equivalent or a homolog thereof as depicted in column 8 of Table II, application no. 17, preferably a homolog or functional equivalent as depicted in column 8 of Table II B, application no. 17, and being depicted in the same respective line as said B3989, and preferably the activity is increased non-targeted, whereby the respective line disclose in column 7 the fine chemical raffinose.

Accordingly, in one embodiment, the molecule which activity is to be increased in the process of the invention is the gene product with an activity as a “b3989-protein”, preferably it is the molecule of section (a) or (b) of this paragraph.

In particular, it was observed that in plants, especially in Arabidopsis thaliana, increasing or generating the activity of a gene product non-targeted with the activity of a “b3989-protein”, preferably being encoded by a gene comprising the nucleic acid sequence SEQ ID NO. 44372, preferably the coding region thereof, conferred the production of or the increase in raffinose compared with the wild type control.

The nucleic acid sequence of B3989 from Escherichia coli, e.g. as shown in the respective line in column 5 of Table I, application no. 17, has been published in B. And the activity of the gene product thereof is the activity of b3989-protein.

Accordingly, in one embodiment, the process of the present invention for producing sucrose in a non-human organism, like a microorganism or a plant or a part thereof, comprises increasing or generating the activity of a gene product with the activity of a gene product conferring the activity of “b3989-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said B3989, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B3989, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B3989, or a functional equivalent or a homolog thereof as depicted in column 8 of Table II, application no. 17, preferably a homolog or functional equivalent as depicted in column 8 of Table II B, application no. 17, and being depicted in the same respective line as said B3989, and preferably the activity is increased non-targeted, whereby the respective line disclose in column 7 the fine chemical sucrose.

Accordingly, in one embodiment, the molecule which activity is to be increased in the process of the invention is the gene product with an activity as a “b3989-protein”, preferably it is the molecule of section (a) or (b) of this paragraph.

In particular, it was observed that in plants, especially in Arabidopsis thaliana, increasing or generating the activity of a gene product non-targeted with the activity of a “b3989-protein”, preferably being encoded by a gene comprising the nucleic acid sequence SEQ ID NO. 44372, preferably the coding region thereof, conferred the production of or the increase in sucrose compared with the wild type control.

The nucleic acid sequence of B4012 from Escherichia coli, e.g. as shown in the respective line in column 5 of Table I, application no. 17, has been published in B. And the activity of the gene product thereof is the activity of acetyltransferase.

Accordingly, in one embodiment, the process of the present invention for producing galactinol in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical galactinol), application no. 17, preferably the coding region thereof, or a homolog or a fragment thereof, and being depicted in the same respective line as said B4012, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B4012, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B4012, or a functional equivalent or a homolog thereof as depicted in column 8 of Table II, application no. 17, preferably a homolog or functional equivalent as depicted in column 8 of Table II B, application no. 17, and being depicted in the same respective line as said B4012, and preferably the activity is increased non-targeted, whereby the respective line disclose in column 7 the fine chemical galactinol.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 44378, preferably the coding region thereof, conferred the production of or the increase in galactinol compared with the wild type control.

The nucleic acid sequence of B4029 from Escherichia coli, e.g. as shown in the respective line in column 5 of Table I, application no. 17, has been published in B. And the activity of the gene product thereof is the activity of b4029-protein.

Accordingly, in one embodiment, the process of the present invention for producing fructose in a non-human organism, like a microorganism or a plant or a part thereof, comprises increasing or generating the activity of a gene product with the activity of a gene product conferring the activity of “b4029-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B4029, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B4029, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B4029, or a functional equivalent or a homolog thereof as depicted in column 8 of Table II, application no. 17, preferably a homolog or functional equivalent as depicted in column 8 of Table II B, application no. 17, and being depicted in the same respective line as said B4029, and preferably the activity is increased non-targeted, whereby the respective line disclose in column 7 the fine chemical fructose.

Accordingly, in one embodiment, the molecule which activity is to be increased in the process of the invention is the gene product with an activity as a “b4029-protein”, preferably it is the molecule of section (a) or (b) of this paragraph.

In particular, it was observed that in plants, especially in Arabidopsis thaliana, increasing or generating the activity of a gene product non-targeted with the activity of a “b4029-protein”, preferably being encoded by a gene comprising the nucleic acid sequence SEQ ID NO. 10740, preferably the coding region thereof, conferred the production of or the increase in fructose compared with the wild type control.

The nucleic acid sequence of B4029 from Escherichia coli, e.g. as shown in the respective line in column 5 of Table I, application no. 17, has been published in B. And the activity of the gene product thereof is the activity of b4029-protein.

Accordingly, in one embodiment, the process of the present invention for producing glucose in a non-human organism, like a microorganism or a plant or a part thereof, comprises increasing or generating the activity of a gene product with the activity of a gene product conferring the activity of “b4029-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B4029, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B4029, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B4029, or a functional equivalent or a homolog thereof as depicted in column 8 of Table II, application no. 17, preferably a homolog or functional equivalent as depicted in column 8 of Table II B, application no. 17, and being depicted in the same respective line as said B4029, and preferably the activity is increased non-targeted, whereby the respective line disclose in column 7 the fine chemical glucose.

Accordingly, in one embodiment, the molecule which activity is to be increased in the process of the invention is the gene product with an activity as a “b4029-protein”, preferably it is the molecule of section (a) or (b) of this paragraph.

In particular, it was observed that in plants, especially in Arabidopsis thaliana, increasing or generating the activity of a gene product non-targeted with the activity of a “b4029-protein”, preferably being encoded by a gene comprising the nucleic acid sequence SEQ ID NO. 10740, preferably the coding region thereof, conferred the production of or the increase in glucose compared with the wild type control.

The nucleic acid sequence of B4029 from Escherichia coli, e.g. as shown in the respective line in column 5 of Table I, application no. 17, has been published in B. And the activity of the gene product thereof is the activity of b4029-protein.

Accordingly, in one embodiment, the process of the present invention for producing sedoheptulose-7-phosphate in a non-human organism, like a microorganism or a plant or a part thereof, comprises increasing or generating the activity of a gene product with the activity of a gene product conferring the activity of “b4029-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical sedoheptulose-7-phosphate), application no. 17, preferably the coding region thereof, or a homolog or a fragment thereof, and being depicted in the same respective line as said B4029, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B4029, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B4029, or a functional equivalent or a homolog thereof as depicted in column 8 of Table II, application no. 17, preferably a homolog or functional equivalent as depicted in column 8 of Table II B, application no. 17, and being depicted in the same respective line as said B4029, and preferably the activity is increased non-targeted, whereby the respective line disclose in column 7 the fine chemical sedoheptulose-7-phosphate.

Accordingly, in one embodiment, the molecule which activity is to be increased in the process of the invention is the gene product with an activity as a “b4029-protein”, preferably it is the molecule of section (a) or (b) of this paragraph.

In particular, it was observed that in plants, especially in Arabidopsis thaliana, increasing or generating the activity of a gene product non-targeted with the activity of a “b4029-protein”, preferably being encoded by a gene comprising the nucleic acid sequence SEQ ID NO. 10740, preferably the coding region thereof, conferred the production of or the increase in sedoheptulose-7-phosphate compared with the wild type control.

The nucleic acid sequence of B4039 from Escherichia coli, e.g. as shown in the respective line in column 5 of Table I, application no. 17, has been published in B. And the activity of the gene product thereof is the activity of chorismate lyase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “chorismate lyase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B4039, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B4039, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B4039, or a functional equivalent or a homolog thereof as depicted in column 8 of Table II, application no. 17, preferably a homolog or functional equivalent as depicted in column 8 of Table II B, application no. 17, and being depicted in the same respective line as said B4039, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “chorismate lyase”, preferably it is the molecule of section (a) or (b) of this paragraph.

In particular, it was observed that in plants, especially in Arabidopsis thaliana, increasing or generating the activity of a gene product plastidic with the activity of a “chorismate lyase”, preferably being encoded by a gene comprising the nucleic acid sequence SEQ ID NO. 146848, preferably the coding region thereof, conferred the production of or the increase in glucose-1-phosphate compared with the wild type control.

The nucleic acid sequence of B4039 from Escherichia coli, e.g. as shown in the respective line in column 5 of Table I, application no. 17, has been published in B. And the activity of the gene product thereof is the activity of chorismate lyase.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “chorismate lyase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B4039, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B4039, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B4039, or a functional equivalent or a homolog thereof as depicted in column 8 of Table II, application no. 17, preferably a homolog or functional equivalent as depicted in column 8 of Table II B, application no. 17, and being depicted in the same respective line as said B4039, and preferably the activity is increased plastidic, whereby the respective line disclose in column 7 the fine chemical 3,4,5-trihydroxypentanoic acid.

Accordingly, in one embodiment, the molecule which activity is to be increased in the process of the invention is the gene product with an activity as a “chorismate lyase”, preferably it is the molecule of section (a) or (b) of this paragraph.

In particular, it was observed that in plants, especially in Arabidopsis thaliana, increasing or generating the activity of a gene product plastidic with the activity of a “chorismate lyase”, preferably being encoded by a gene comprising the nucleic acid sequence SEQ ID NO. 146848, preferably the coding region thereof, conferred the production of or the increase in 3,4,5-trihydroxypentanoic acid compared with the wild type control.

The nucleic acid sequence of B4050 from Escherichia coli, e.g. as shown in the respective line in column 5 of Table I, application no. 17, has been published in B. And the activity of the gene product thereof is the activity of b4050-protein.

Accordingly, in one embodiment, the process of the present invention for producing fructose in a non-human organism, like a microorganism or a plant or a part thereof, comprises increasing or generating the activity of a gene product with the activity of a gene product conferring the activity of “b4050-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B4050, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B4050, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B4050, or a functional equivalent or a homolog thereof as depicted in column 8 of Table II, application no. 17, preferably a homolog or functional equivalent as depicted in column 8 of Table II B, application no. 17, and being depicted in the same respective line as said B4050, and preferably the activity is increased non-targeted, whereby the respective line disclose in column 7 the fine chemical fructose.

Accordingly, in one embodiment, the molecule which activity is to be increased in the process of the invention is the gene product with an activity as a “b4050-protein”, preferably it is the molecule of section (a) or (b) of this paragraph.

In particular, it was observed that in plants, especially in Arabidopsis thaliana, increasing or generating the activity of a gene product non-targeted with the activity of a “b4050-protein”, preferably being encoded by a gene comprising the nucleic acid sequence SEQ ID NO. 106532, preferably the coding region thereof, conferred the production of or the increase in fructose compared with the wild type control.

The nucleic acid sequence of B4050 from Escherichia coli, e.g. as shown in the respective line in column 5 of Table I, application no. 17, has been published in B. And the activity of the gene product thereof is the activity of b4050-protein.

Accordingly, in one embodiment, the process of the present invention for producing glucose in a non-human organism, like a microorganism or a plant or a part thereof, comprises increasing or generating the activity of a gene product with the activity of a gene product conferring the activity of “b4050-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B4050, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B4050, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B4050, or a functional equivalent or a homolog thereof as depicted in column 8 of Table II, application no. 17, preferably a homolog or functional equivalent as depicted in column 8 of Table II B, application no. 17, and being depicted in the same respective line as said B4050, and preferably the activity is increased non-targeted, whereby the respective line disclose in column 7 the fine chemical glucose.

Accordingly, in one embodiment, the molecule which activity is to be increased in the process of the invention is the gene product with an activity as a “b4050-protein”, preferably it is the molecule of section (a) or (b) of this paragraph.

In particular, it was observed that in plants, especially in Arabidopsis thaliana, increasing or generating the activity of a gene product non-targeted with the activity of a “b4050-protein”, preferably being encoded by a gene comprising the nucleic acid sequence SEQ ID NO. 106532, preferably the coding region thereof, conferred the production of or the increase in glucose compared with the wild type control.

The nucleic acid sequence of B4050 from Escherichia coli, e.g. as shown in the respective line in column 5 of Table I, application no. 17, has been published in B. And the activity of the gene product thereof is the activity of b4050-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “b4050-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B4050, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B4050, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B4050, or a functional equivalent or a homolog thereof as depicted in column 8 of Table II, application no. 17, preferably a homolog or functional equivalent as depicted in column 8 of Table II B, application no. 17, and being depicted in the same respective line as said B4050, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “b4050-protein”, preferably it is the molecule of section (a) or (b) of this paragraph.

In particular, it was observed that in plants, especially in Arabidopsis thaliana, increasing or generating the activity of a gene product non-targeted with the activity of a “b4050-protein”, preferably being encoded by a gene comprising the nucleic acid sequence SEQ ID NO. 106532, preferably the coding region thereof, conferred the production of or the increase in myo-inositol compared with the wild type control.

The nucleic acid sequence of B4056 from Escherichia coli, e.g. as shown in the respective line in column 5 of Table I, application no. 17, has been published in B. And the activity of the gene product thereof is the activity of b4056-protein.

Accordingly, in one embodiment, the process of the present invention for producing fructose in a non-human organism, like a microorganism or a plant or a part thereof, comprises increasing or generating the activity of a gene product with the activity of a gene product conferring the activity of “b4056-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B4056, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B4056, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B4056, or a functional equivalent or a homolog thereof as depicted in column 8 of Table II, application no. 17, preferably a homolog or functional equivalent as depicted in column 8 of Table II B, application no. 17, and being depicted in the same respective line as said B4056, and preferably the activity is increased non-targeted, whereby the respective line disclose in column 7 the fine chemical fructose.

Accordingly, in one embodiment, the molecule which activity is to be increased in the process of the invention is the gene product with an activity as a “b4056-protein”, preferably it is the molecule of section (a) or (b) of this paragraph.

In particular, it was observed that in plants, especially in Arabidopsis thaliana, increasing or generating the activity of a gene product non-targeted with the activity of a “b4056-protein”, preferably being encoded by a gene comprising the nucleic acid sequence SEQ ID NO. 94542, preferably the coding region thereof, conferred the production of or the increase in fructose compared with the wild type control.

The nucleic acid sequence of B4069 from Escherichia coli, e.g. as shown in the respective line in column 5 of Table I, application no. 17, has been published in B. And the activity of the gene product thereof is the activity of acetyl-coenzyme A synthetase.

Accordingly, in one embodiment, the process of the present invention for producing galactinol in a non-human organism, like a microorganism or a plant or a part thereof, comprises increasing or generating the activity of a gene product with the activity of a gene product conferring the activity of “acetyl-coenzyme A synthetase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical galactinol), application no. 17, preferably the coding region thereof, or a homolog or a fragment thereof, and being depicted in the same respective line as said B4069, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B4069, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B4069, or a functional equivalent or a homolog thereof as depicted in column 8 of Table II, application no. 17, preferably a homolog or functional equivalent as depicted in column 8 of Table II B, application no. 17, and being depicted in the same respective line as said B4069, and preferably the activity is increased plastidic, whereby the respective line disclose in column 7 the fine chemical galactinol.

Accordingly, in one embodiment, the molecule which activity is to be increased in the process of the invention is the gene product with an activity as a “acetyl-coenzyme A synthetase”, preferably it is the molecule of section (a) or (b) of this paragraph.

In particular, it was observed that in plants, especially in Arabidopsis thaliana, increasing or generating the activity of a gene product plastidic with the activity of a “acetyl-coenzyme A synthetase”, preferably being encoded by a gene comprising the nucleic acid sequence SEQ ID NO. 126421, preferably the coding region thereof, conferred the production of or the increase in galactinol compared with the wild type control.

The nucleic acid sequence of B4189 from Escherichia coli, e.g. as shown in the respective line in column 5 of Table I, application no. 17, has been published in B. And the activity of the gene product thereof is the activity of lipoprotein precursor.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “lipoprotein precursor”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said B4189, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B4189, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B4189, or a functional equivalent or a homolog thereof as depicted in column 8 of Table II, application no. 17, preferably a homolog or functional equivalent as depicted in column 8 of Table II B, application no. 17, and being depicted in the same respective line as said B4189, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “lipoprotein precursor”, preferably it is the molecule of section (a) or (b) of this paragraph.

In particular, it was observed that in plants, especially in Arabidopsis thaliana, increasing or generating the activity of a gene product non-targeted with the activity of a “lipoprotein precursor”, preferably being encoded by a gene comprising the nucleic acid sequence SEQ ID NO. 146921, preferably the coding region thereof, conferred the production of or the increase in glucose-1-phosphate compared with the wild type control.

The nucleic acid sequence of B4189 from Escherichia coli, e.g. as shown in the respective line in column 5 of Table I, application no. 17, has been published in B. And the activity of the gene product thereof is the activity of lipoprotein precursor.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “lipoprotein precursor”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said B4189, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B4189, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B4189, or a functional equivalent or a homolog thereof as depicted in column 8 of Table II, application no. 17, preferably a homolog or functional equivalent as depicted in column 8 of Table II B, application no. 17, and being depicted in the same respective line as said B4189, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “lipoprotein precursor”, preferably it is the molecule of section (a) or (b) of this paragraph.

In particular, it was observed that in plants, especially in Arabidopsis thaliana, increasing or generating the activity of a gene product non-targeted with the activity of a “lipoprotein precursor”, preferably being encoded by a gene comprising the nucleic acid sequence SEQ ID NO. 146921, preferably the coding region thereof, conferred the production of or the increase in 3,4,5-trihydroxypentanoic acid compared with the wild type control.

The nucleic acid sequence of B4250 from Escherichia coli, e.g. as shown in the respective line in column 5 of Table I, application no. 17, has been published in B. And the activity of the gene product thereof is the activity of b4250-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “b4250-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said B4250, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B4250, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B4250, or a functional equivalent or a homolog thereof as depicted in column 8 of Table II, application no. 17, preferably a homolog or functional equivalent as depicted in column 8 of Table II B, application no. 17, and being depicted in the same respective line as said B4250, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “b4250-protein”, preferably it is the molecule of section (a) or (b) of this paragraph.

In particular, it was observed that in plants, especially in Arabidopsis thaliana, increasing or generating the activity of a gene product non-targeted with the activity of a “b4250-protein”, preferably being encoded by a gene comprising the nucleic acid sequence SEQ ID NO. 94735, preferably the coding region thereof, conferred the production of or the increase in myo-inositol compared with the wild type control.

The nucleic acid sequence of B4250 from Escherichia coli, e.g. as shown in the respective line in column 5 of Table I, application no. 17, has been published in B. And the activity of the gene product thereof is the activity of b4250-protein.

Accordingly, in one embodiment, the process of the present invention for producing raffinose in a non-human organism, like a microorganism or a plant or a part thereof, comprises increasing or generating the activity of a gene product with the activity of a gene product conferring the activity of “b4250-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said B4250, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B4250, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B4250, or a functional equivalent or a homolog thereof as depicted in column 8 of Table II, application no. 17, preferably a homolog or functional equivalent as depicted in column 8 of Table II B, application no. 17, and being depicted in the same respective line as said B4250, and preferably the activity is increased non-targeted, whereby the respective line disclose in column 7 the fine chemical raffinose.

Accordingly, in one embodiment, the molecule which activity is to be increased in the process of the invention is the gene product with an activity as a “b4250-protein”, preferably it is the molecule of section (a) or (b) of this paragraph.

In particular, it was observed that in plants, especially in Arabidopsis thaliana, increasing or generating the activity of a gene product non-targeted with the activity of a “b4250-protein”, preferably being encoded by a gene comprising the nucleic acid sequence SEQ ID NO. 94735, preferably the coding region thereof, conferred the production of or the increase in raffinose compared with the wild type control.

The nucleic acid sequence of B4256 from Escherichia coli, e.g. as shown in the respective line in column 5 of Table I, application no. 17, has been published in B. And the activity of the gene product thereof is the activity of acetyltransferase.

Accordingly, in one embodiment, the process of the present invention for producing fructose in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B4256, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B4256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B4256, or a functional equivalent or a homolog thereof as depicted in column 8 of Table II, application no. 17, preferably a homolog or functional equivalent as depicted in column 8 of Table II B, application no. 17, and being depicted in the same respective line as said B4256, and preferably the activity is increased non-targeted, whereby the respective line disclose in column 7 the fine chemical fructose.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 45321, preferably the coding region thereof, conferred the production of or the increase in fructose compared with the wild type control.

The nucleic acid sequence of B4256 from Escherichia coli, e.g. as shown in the respective line in column 5 of Table I, application no. 17, has been published in B. And the activity of the gene product thereof is the activity of acetyltransferase.

Accordingly, in one embodiment, the process of the present invention for producing glucose in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said B4256, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B4256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B4256, or a functional equivalent or a homolog thereof as depicted in column 8 of Table II, application no. 17, preferably a homolog or functional equivalent as depicted in column 8 of Table II B, application no. 17, and being depicted in the same respective line as said B4256, and preferably the activity is increased non-targeted, whereby the respective line disclose in column 7 the fine chemical glucose.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 45321, preferably the coding region thereof, conferred the production of or the increase in glucose compared with the wild type control.

The nucleic acid sequence of B4321 from Escherichia coli, e.g. as shown in the respective line in column 5 of Table I, application no. 17, has been published in B. And the activity of the gene product thereof is the activity of gluconate transport system permease 3.

Accordingly, in one embodiment, the process of the present invention for producing fructose in a non-human organism, like a microorganism or a plant or a part thereof, comprises increasing or generating the activity of a gene product with the activity of a gene product conferring the activity of “gluconate transport system permease 3”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said B4321, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B4321, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B4321, or a functional equivalent or a homolog thereof as depicted in column 8 of Table II, application no. 17, preferably a homolog or functional equivalent as depicted in column 8 of Table II B, application no. 17, and being depicted in the same respective line as said B4321, and preferably the activity is increased non-targeted, whereby the respective line disclose in column 7 the fine chemical fructose.

Accordingly, in one embodiment, the molecule which activity is to be increased in the process of the invention is the gene product with an activity as a “gluconate transport system permease 3”, preferably it is the molecule of section (a) or (b) of this paragraph.

In particular, it was observed that in plants, especially in Arabidopsis thaliana, increasing or generating the activity of a gene product non-targeted with the activity of a “gluconate transport system permease 3”, preferably being encoded by a gene comprising the nucleic acid sequence SEQ ID NO. 45394, preferably the coding region thereof, conferred the production of or the increase in fructose compared with the wild type control.

The nucleic acid sequence of B4384 from Escherichia coli, e.g. as shown in the respective line in column 5 of Table I, application no. 17, has been published in B. And the activity of the gene product thereof is the activity of purine-nucleoside phosphorylase.

Accordingly, in one embodiment, the process of the present invention for producing galactinol in a non-human organism, like a microorganism or a plant or a part thereof, comprises increasing or generating the activity of a gene product with the activity of a gene product conferring the activity of “purine-nucleoside phosphorylase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical galactinol), application no. 17, preferably the coding region thereof, or a homolog or a fragment thereof, and being depicted in the same respective line as said B4384, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said B4384, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said B4384, or a functional equivalent or a homolog thereof as depicted in column 8 of Table II, application no. 17, preferably a homolog or functional equivalent as depicted in column 8 of Table II B, application no. 17, and being depicted in the same respective line as said B4384, and preferably the activity is increased non-targeted, whereby the respective line disclose in column 7 the fine chemical galactinol.

Accordingly, in one embodiment, the molecule which activity is to be increased in the process of the invention is the gene product with an activity as a “purine-nucleoside phosphorylase”, preferably it is the molecule of section (a) or (b) of this paragraph.

In particular, it was observed that in plants, especially in Arabidopsis thaliana, increasing or generating the activity of a gene product non-targeted with the activity of a “purine-nucleoside phosphorylase”, preferably being encoded by a gene comprising the nucleic acid sequence SEQ ID NO. 45556, preferably the coding region thereof, conferred the production of or the increase in galactinol compared with the wild type control.

The nucleic acid sequence of D90900 from Synechocystis sp., e.g. as shown in the respective line in column 5 of Table I, application no. 17, has been published in C. And the activity of the gene product thereof is the activity of lysine decarboxylase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “lysine decarboxylase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said D90900, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said D90900, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said D90900, or a functional equivalent or a homolog thereof as depicted in column 8 of Table II, application no. 17, preferably a homolog or functional equivalent as depicted in column 8 of Table II B, application no. 17, and being depicted in the same respective line as said D90900, and preferably the activity is increased plastidic, whereby the respective line disclose in column 7 the fine chemical myo-inositol.

Accordingly, in one embodiment, the molecule which activity is to be increased in the process of the invention is the gene product with an activity as a “lysine decarboxylase”, preferably it is the molecule of section (a) or (b) of this paragraph.

In particular, it was observed that in plants, especially in Arabidopsis thaliana, increasing or generating the activity of a gene product plastidic with the activity of a “lysine decarboxylase”, preferably being encoded by a gene comprising the nucleic acid sequence SEQ ID NO. 84079, preferably the coding region thereof, conferred the production of or the increase in myo-inositol compared with the wild type control.

The nucleic acid sequence of GM02LC12622 from Glycine max, e.g. as shown in the respective line in column 5 of Table I, application no. 17, is unpublished. And the activity of the gene product thereof is the activity of glutaredoxin.

Accordingly, in one embodiment, the process of the present invention for producing fructose in a non-human organism, like a microorganism or a plant or a part thereof, comprises increasing or generating the activity of a gene product with the activity of a gene product conferring the activity of “glutaredoxin”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said GM02LC12622, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said GM02LC12622, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said GM02LC12622, or a functional equivalent or a homolog thereof as depicted in column 8 of Table II, application no. 17, preferably a homolog or functional equivalent as depicted in column 8 of Table II B, application no. 17, and being depicted in the same respective line as said GM02LC12622, and preferably the activity is increased non-targeted, whereby the respective line disclose in column 7 the fine chemical fructose.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 10811, preferably the coding region thereof, conferred the production of or the increase in fructose compared with the wild type control.

The nucleic acid sequence of GM02LC12622 from Glycine max, e.g. as shown in the respective line in column 5 of Table I, application no. 17, is unpublished. And the activity of the gene product thereof is the activity of glutaredoxin.

Accordingly, in one embodiment, the process of the present invention for producing glucose in a non-human organism, like a microorganism or a plant or a part thereof, comprises increasing or generating the activity of a gene product with the activity of a gene product conferring the activity of “glutaredoxin”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said GM02LC12622, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said GM02LC12622, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said GM02LC12622, or a functional equivalent or a homolog thereof as depicted in column 8 of Table II, application no. 17, preferably a homolog or functional equivalent as depicted in column 8 of Table II B, application no. 17, and being depicted in the same respective line as said GM02LC12622, and preferably the activity is increased non-targeted, whereby the respective line disclose in column 7 the fine chemical glucose.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 10811, preferably the coding region thereof, conferred the production of or the increase in glucose compared with the wild type control.

The nucleic acid sequence of GM02LC12622 from Glycine max, e.g. as shown in the respective line in column 5 of Table I, application no. 17, is unpublished. And the activity of the gene product thereof is the activity of glutaredoxin.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “glutaredoxin”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said GM02LC12622, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said GM02LC12622, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said GM02LC12622, or a functional equivalent or a homolog thereof as depicted in column 8 of Table II, application no. 17, preferably a homolog or functional equivalent as depicted in column 8 of Table II B, application no. 17, and being depicted in the same respective line as said GM02LC12622, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 10811, preferably the coding region thereof, conferred the production of or the increase in glucose-1-phosphate compared with the wild type control.

The nucleic acid sequence of GM02LC12622 from Glycine max, e.g. as shown in the respective line in column 5 of Table I, application no. 17, is unpublished. And the activity of the gene product thereof is the activity of glutaredoxin.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “glutaredoxin”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said GM02LC12622, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said GM02LC12622, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said GM02LC12622, or a functional equivalent or a homolog thereof as depicted in column 8 of Table II, application no. 17, preferably a homolog or functional equivalent as depicted in column 8 of Table II B, application no. 17, and being depicted in the same respective line as said GM02LC12622, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 10811, preferably the coding region thereof, conferred the production of or the increase in myo-inositol compared with the wild type control.

The nucleic acid sequence of GM02LC12622 from Glycine max, e.g. as shown in the respective line in column 5 of Table I, application no. 17, is unpublished. And the activity of the gene product thereof is the activity of glutaredoxin.

Accordingly, in one embodiment, the process of the present invention for producing sucrose in a non-human organism, like a microorganism or a plant or a part thereof, comprises increasing or generating the activity of a gene product with the activity of a gene product conferring the activity of “glutaredoxin”, especially from Glycine max or its functional equivalent or its homolog, e.g.

the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said GM02LC12622, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said GM02LC12622, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said GM02LC12622, or a functional equivalent or a homolog thereof as depicted in column 8 of Table II, application no. 17, preferably a homolog or functional equivalent as depicted in column 8 of Table II B, application no. 17, and being depicted in the same respective line as said GM02LC12622, and preferably the activity is increased non-targeted, whereby the respective line disclose in column 7 the fine chemical sucrose.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 10811, preferably the coding region thereof, conferred the production of or the increase in sucrose compared with the wild type control.

The nucleic acid sequence of GM02LC19289 from Glycine max, e.g. as shown in the respective line in column 5 of Table I, application no. 17, is unpublished. And the activity of the gene product thereof is the activity of transcription factor.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said GM02LC19289, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said GM02LC19289, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said GM02LC19289, or a functional equivalent or a homolog thereof as depicted in column 8 of Table II, application no. 17, preferably a homolog or functional equivalent as depicted in column 8 of Table II B, application no. 17, and being depicted in the same respective line as said GM02LC19289, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 46751, preferably the coding region thereof, conferred the production of or the increase in glucose-1-phosphate compared with the wild type control.

The nucleic acid sequence of GM02LC19289 from Glycine max, e.g. as shown in the respective line in column 5 of Table I, application no. 17, is unpublished. And the activity of the gene product thereof is the activity of transcription factor.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said GM02LC19289, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said GM02LC19289, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said GM02LC19289, or a functional equivalent or a homolog thereof as depicted in column 8 of Table II, application no. 17, preferably a homolog or functional equivalent as depicted in column 8 of Table II B, application no. 17, and being depicted in the same respective line as said GM02LC19289, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 46751, preferably the coding region thereof, conferred the production of or the increase in 3,4,5-trihydroxypentanoic acid compared with the wild type control.

The nucleic acid sequence of GM02LC21368 from Glycine max, e.g. as shown in the respective line in column 5 of Table I, application no. 17, is unpublished. And the activity of the gene product thereof is the activity of transcription factor.

Accordingly, in one embodiment, the process of the present invention for producing fructose in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said GM02LC21368, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said GM02LC21368, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said GM02LC21368, or a functional equivalent or a homolog thereof as depicted in column 8 of Table II, application no. 17, preferably a homolog or functional equivalent as depicted in column 8 of Table II B, application no. 17, and being depicted in the same respective line as said GM02LC21368, and preferably the activity is increased non-targeted, whereby the respective line disclose in column 7 the fine chemical fructose.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 84198, preferably the coding region thereof, conferred the production of or the increase in fructose compared with the wild type control.

The nucleic acid sequence of GM02LC38418 from Glycine max, e.g. as shown in the respective line in column 5 of Table I, application no. 17, is unpublished. And the activity of the gene product thereof is the activity of GM02LC38418-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “GM02LC38418-protein”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said GM02LC38418, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said GM02LC38418, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said GM02LC38418, or a functional equivalent or a homolog thereof as depicted in column 8 of Table II, application no. 17, preferably a homolog or functional equivalent as depicted in column 8 of Table II B, application no. 17, and being depicted in the same respective line as said GM02LC38418, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “GM02LC38418-protein”, preferably it is the molecule of section (a) or (b) of this paragraph.

In particular, it was observed that in plants, especially in Arabidopsis thaliana, increasing or generating the activity of a gene product non-targeted with the activity of a “GM02LC38418-protein”, preferably being encoded by a gene comprising the nucleic acid sequence SEQ ID NO. 116312, preferably the coding region thereof, conferred the production of or the increase in glucose-1-phosphate compared with the wild type control.

The nucleic acid sequence of GM02LC44512 from Glycine max, e.g. as shown in the respective line in column 5 of Table I, application no. 17, is unpublished. And the activity of the gene product thereof is the activity of cyclin D.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “cyclin D”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said GM02LC44512, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said GM02LC44512, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said GM02LC44512, or a functional equivalent or a homolog thereof as depicted in column 8 of Table II, application no. 17, preferably a homolog or functional equivalent as depicted in column 8 of Table II B, application no. 17, and being depicted in the same respective line as said GM02LC44512, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 46850, preferably the coding region thereof, conferred the production of or the increase in glucose-1-phosphate compared with the wild type control.

The nucleic acid sequence of GM02LC44512 from Glycine max, e.g. as shown in the respective line in column 5 of Table I, application no. 17, is unpublished. And the activity of the gene product thereof is the activity of cyclin D.

Accordingly, in one embodiment, the process of the present invention for producing raffinose in a non-human organism, like a microorganism or a plant or a part thereof, comprises increasing or generating the activity of a gene product with the activity of a gene product conferring the activity of “cyclin D”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said GM02LC44512, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said GM02LC44512, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said GM02LC44512, or a functional equivalent or a homolog thereof as depicted in column 8 of Table II, application no. 17, preferably a homolog or functional equivalent as depicted in column 8 of Table II B, application no. 17, and being depicted in the same respective line as said GM02LC44512, and preferably the activity is increased non-targeted, whereby the respective line disclose in column 7 the fine chemical raffinose.

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 46850, preferably the coding region thereof, conferred the production of or the increase in raffinose compared with the wild type control.

The nucleic acid sequence of GM02LC44512 from Glycine max, e.g. as shown in the respective line in column 5 of Table I, application no. 17, is unpublished. And the activity of the gene product thereof is the activity of cyclin D.

Accordingly, in one embodiment, the process of the present invention for producing sucrose in a non-human organism, like a microorganism or a plant or a part thereof, comprises increasing or generating the activity of a gene product with the activity of a gene product conferring the activity of “cyclin D”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said GM02LC44512, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said GM02LC44512, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said GM02LC44512, or a functional equivalent or a homolog thereof as depicted in column 8 of Table II, application no. 17, preferably a homolog or functional equivalent as depicted in column 8 of Table II B, application no. 17, and being depicted in the same respective line as said GM02LC44512, and preferably the activity is increased non-targeted, whereby the respective line disclose in column 7 the fine chemical sucrose.

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 46850, preferably the coding region thereof, conferred the production of or the increase in sucrose compared with the wild type control.

The nucleic acid sequence of GM02LC46 from Glycine max, e.g. as shown in the respective line in column 5 of Table I, application no. 17, is unpublished. And the activity of the gene product thereof is the activity of GM02LC46-protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “GM02LC46-protein”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said GM02LC46, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said GM02LC46, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said GM02LC46, or a functional equivalent or a homolog thereof as depicted in column 8 of Table II, application no. 17, preferably a homolog or functional equivalent as depicted in column 8 of Table II B, application no. 17, and being depicted in the same respective line as said GM02LC46, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “GM02LC46-protein”, preferably it is the molecule of section (a) or (b) of this paragraph.

In particular, it was observed that in plants, especially in Arabidopsis thaliana, increasing or generating the activity of a gene product non-targeted with the activity of a “GM02LC46-protein”, preferably being encoded by a gene comprising the nucleic acid sequence SEQ ID NO. 47026, preferably the coding region thereof, conferred the production of or the increase in 3,4,5-trihydroxypentanoic acid compared with the wild type control.

The nucleic acid sequence of GM02LC6021 from Glycine max, e.g. as shown in the respective line in column 5 of Table I, application no. 17, is unpublished. And the activity of the gene product thereof is the activity of transcription factor.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said GM02LC6021, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said GM02LC6021, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said GM02LC6021, or a functional equivalent or a homolog thereof as depicted in column 8 of Table II, application no. 17, preferably a homolog or functional equivalent as depicted in column 8 of Table II B, application no. 17, and being depicted in the same respective line as said GM02LC6021, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 47105, preferably the coding region thereof, conferred the production of or the increase in 3,4,5-trihydroxypentanoic acid compared with the wild type control.

The nucleic acid sequence of Sll0222 from Synechocystis sp., e.g. as shown in the respective line in column 5 of Table I, application no. 17, has been published in C. And the activity of the gene product thereof is the activity of alkaline phosphatase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “alkaline phosphatase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said Sll0222, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll0222, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll0222, or a functional equivalent or a homolog thereof as depicted in column 8 of Table II, application no. 17, preferably a homolog or functional equivalent as depicted in column 8 of Table II B, application no. 17, and being depicted in the same respective line as said Sll0222, and preferably the activity is increased plastidic, whereby the respective line disclose in column 7 the fine chemical myo-inositol.

Accordingly, in one embodiment, the molecule which activity is to be increased in the process of the invention is the gene product with an activity as a “alkaline phosphatase”, preferably it is the molecule of section (a) or (b) of this paragraph.

In particular, it was observed that in plants, especially in Arabidopsis thaliana, increasing or generating the activity of a gene product plastidic with the activity of a “alkaline phosphatase”, preferably being encoded by a gene comprising the nucleic acid sequence SEQ ID NO. 146939, preferably the coding region thereof, conferred the production of or the increase in myo-inositol compared with the wild type control.

The nucleic acid sequence of Sll0248 from Synechocystis sp., e.g. as shown in the respective line in column 5 of Table I, application no. 17, has been published in C. And the activity of the gene product thereof is the activity of flavodoxin.

Accordingly, in one embodiment, the process of the present invention for producing verbascose in a non-human organism, like a microorganism or a plant or a part thereof, comprises increasing or generating the activity of a gene product with the activity of a gene product conferring the activity of “flavodoxin”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical verbascose), application no. 17, preferably the coding region thereof, or a homolog or a fragment thereof, and being depicted in the same respective line as said Sll0248, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll0248, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll0248, or a functional equivalent or a homolog thereof as depicted in column 8 of Table II, application no. 17, preferably a homolog or functional equivalent as depicted in column 8 of Table II B, application no. 17, and being depicted in the same respective line as said Sll0248, and preferably the activity is increased plastidic, whereby the respective line disclose in column 7 the fine chemical verbascose.

Accordingly, in one embodiment, the molecule which activity is to be increased in the process of the invention is the gene product with an activity as a “flavodoxin”, preferably it is the molecule of section (a) or (b) of this paragraph.

In particular, it was observed that in plants, especially in Arabidopsis thaliana, increasing or generating the activity of a gene product plastidic with the activity of a “flavodoxin”, preferably being encoded by a gene comprising the nucleic acid sequence SEQ ID NO. 49143, preferably the coding region thereof, conferred the production of or the increase in verbascose compared with the wild type control.

The nucleic acid sequence of Sll0254 from Synechocystis sp., e.g. as shown in the respective line in column 5 of Table I, application no. 17, has been published in C. And the activity of the gene product thereof is the activity of sll0254-protein.

Accordingly, in one embodiment, the process of the present invention for producing sucrose in a non-human organism, like a microorganism or a plant or a part thereof, comprises increasing or generating the activity of a gene product with the activity of a gene product conferring the activity of “sll0254-protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said Sll0254, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll0254, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll0254, or a functional equivalent or a homolog thereof as depicted in column 8 of Table II, application no. 17, preferably a homolog or functional equivalent as depicted in column 8 of Table II B, application no. 17, and being depicted in the same respective line as said Sll0254, and preferably the activity is increased mitochondrial, whereby the respective line disclose in column 7 the fine chemical sucrose.

Accordingly, in one embodiment, the molecule which activity is to be increased in the process of the invention is the gene product with an activity as a “sll0254-protein”, preferably it is the molecule of section (a) or (b) of this paragraph.

In particular, it was observed that in plants, especially in Arabidopsis thaliana, increasing or generating the activity of a gene product mitochondrial with the activity of a “sll0254-protein”, preferably being encoded by a gene comprising the nucleic acid sequence SEQ ID NO. 49800, preferably the coding region thereof, conferred the production of or the increase in sucrose compared with the wild type control.

The nucleic acid sequence of Sll0281 from Synechocystis sp., e.g. as shown in the respective line in column 5 of Table I, application no. 17, has been published in C. And the activity of the gene product thereof is the activity of sll0281-protein.

Accordingly, in one embodiment, the process of the present invention for producing verbascose in a non-human organism, like a microorganism or a plant or a part thereof, comprises increasing or generating the activity of a gene product with the activity of a gene product conferring the activity of “sll0281-protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical verbascose), application no. 17, preferably the coding region thereof, or a homolog or a fragment thereof, and being depicted in the same respective line as said Sll0281, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll0281, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll0281, or a functional equivalent or a homolog thereof as depicted in column 8 of Table II, application no. 17, preferably a homolog or functional equivalent as depicted in column 8 of Table II B, application no. 17, and being depicted in the same respective line as said Sll0281, and preferably the activity is increased plastidic, whereby the respective line disclose in column 7 the fine chemical verbascose.

Accordingly, in one embodiment, the molecule which activity is to be increased in the process of the invention is the gene product with an activity as a “sll0281-protein”, preferably it is the molecule of section (a) or (b) of this paragraph.

In particular, it was observed that in plants, especially in Arabidopsis thaliana, increasing or generating the activity of a gene product plastidic with the activity of a “sll0281-protein”, preferably being encoded by a gene comprising the nucleic acid sequence SEQ ID NO. 84865, preferably the coding region thereof, conferred the production of or the increase in verbascose compared with the wild type control.

The nucleic acid sequence of Sll0354 from Synechocystis sp., e.g. as shown in the respective line in column 5 of Table I, application no. 17, has been published in C. And the activity of the gene product thereof is the activity of sll0354-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “sll0354-protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said Sll0354, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll0354, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll0354, or a functional equivalent or a homolog thereof as depicted in column 8 of Table II, application no. 17, preferably a homolog or functional equivalent as depicted in column 8 of Table II B, application no. 17, and being depicted in the same respective line as said Sll0354, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “sll0354-protein”, preferably it is the molecule of section (a) or (b) of this paragraph.

In particular, it was observed that in plants, especially in Arabidopsis thaliana, increasing or generating the activity of a gene product non-targeted with the activity of a “sll0354-protein”, preferably being encoded by a gene comprising the nucleic acid sequence SEQ ID NO. 50070, preferably the coding region thereof, conferred the production of or the increase in myo-inositol compared with the wild type control.

The nucleic acid sequence of Sll0368 from Synechocystis sp., e.g. as shown in the respective line in column 5 of Table I, application no. 17, has been published in C. And the activity of the gene product thereof is the activity of bifunctional protein (phosphoribosyltransferase and regulatory protein).

Accordingly, in one embodiment, the process of the present invention for producing myo-inositol in a non-human organism, like a microorganism or a plant or a part thereof, comprises increasing or generating the activity of a gene product with the activity of a gene product conferring the activity of “bifunctional protein (phosphoribosyltransferase and regulatory protein)”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said Sll0368, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll0368, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll0368, or a functional equivalent or a homolog thereof as depicted in column 8 of Table II, application no. 17, preferably a homolog or functional equivalent as depicted in column 8 of Table II B, application no. 17, and being depicted in the same respective line as said Sll0368, and preferably the activity is increased mitochondrial, whereby the respective line disclose in column 7 the fine chemical myo-inositol.

Accordingly, in one embodiment, the molecule which activity is to be increased in the process of the invention is the gene product with an activity as a “bifunctional protein (phosphoribosyltransferase and regulatory protein)”, preferably it is the molecule of section (a) or (b) of this paragraph.

In particular, it was observed that in plants, especially in Arabidopsis thaliana, increasing or generating the activity of a gene product mitochondrial with the activity of a “bifunctional protein (phosphoribosyltransferase and regulatory protein)”, preferably being encoded by a gene comprising the nucleic acid sequence SEQ ID NO. 106920, preferably the coding region thereof, conferred the production of or the increase in myo-inositol compared with the wild type control.

The nucleic acid sequence of Sll0418 from Synechocystis sp., e.g. as shown in the respective line in column 5 of Table I, application no. 17, has been published in C. And the activity of the gene product thereof is the activity of sll0418-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “sll0418-protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Sll0418, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll0418, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll0418, or a functional equivalent or a homolog thereof as depicted in column 8 of Table II, application no. 17, preferably a homolog or functional equivalent as depicted in column 8 of Table II B, application no. 17, and being depicted in the same respective line as said Sll0418, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “sll0418-protein”, preferably it is the molecule of section (a) or (b) of this paragraph.

In particular, it was observed that in plants, especially in Arabidopsis thaliana, increasing or generating the activity of a gene product non-targeted with the activity of a “sll0418-protein”, preferably being encoded by a gene comprising the nucleic acid sequence SEQ ID NO. 94942, preferably the coding region thereof, conferred the production of or the increase in glucose-1-phosphate compared with the wild type control.

The nucleic acid sequence of Sll0418 from Synechocystis sp., e.g. as shown in the respective line in column 5 of Table I, application no. 17, has been published in C. And the activity of the gene product thereof is the activity of sll0418-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “sll0418-protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said Sll0418, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll0418, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll0418, or a functional equivalent or a homolog thereof as depicted in column 8 of Table II, application no. 17, preferably a homolog or functional equivalent as depicted in column 8 of Table II B, application no. 17, and being depicted in the same respective line as said Sll0418, and preferably the activity is increased mitochondrial, whereby the respective line disclose in column 7 the fine chemical myo-inositol.

Accordingly, in one embodiment, the molecule which activity is to be increased in the process of the invention is the gene product with an activity as a “sll0418-protein”, preferably it is the molecule of section (a) or (b) of this paragraph.

In particular, it was observed that in plants, especially in Arabidopsis thaliana, increasing or generating the activity of a gene product mitochondrial with the activity of a “sll0418-protein”, preferably being encoded by a gene comprising the nucleic acid sequence SEQ ID NO. 94942, preferably the coding region thereof, conferred the production of or the increase in myo-inositol compared with the wild type control.

The nucleic acid sequence of Sll0418 from Synechocystis sp., e.g. as shown in the respective line in column 5 of Table I, application no. 17, has been published in C. And the activity of the gene product thereof is the activity of sll0418-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “sll0418-protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said Sll0418, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll0418, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll0418, or a functional equivalent or a homolog thereof as depicted in column 8 of Table II, application no. 17, preferably a homolog or functional equivalent as depicted in column 8 of Table II B, application no. 17, and being depicted in the same respective line as said Sll0418, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “sll0418-protein”, preferably it is the molecule of section (a) or (b) of this paragraph.

In particular, it was observed that in plants, especially in Arabidopsis thaliana, increasing or generating the activity of a gene product non-targeted with the activity of a “sll0418-protein”, preferably being encoded by a gene comprising the nucleic acid sequence SEQ ID NO. 94942, preferably the coding region thereof, conferred the production of or the increase in myo-inositol compared with the wild type control.

The nucleic acid sequence of Sll0418 from Synechocystis sp., e.g. as shown in the respective line in column 5 of Table I, application no. 17, has been published in C. And the activity of the gene product thereof is the activity of sll0418-protein.

Accordingly, in one embodiment, the process of the present invention for producing saccharic acid in a non-human organism, like a microorganism or a plant or a part thereof, comprises increasing or generating the activity of a gene product with the activity of a gene product conferring the activity of “sll0418-protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical saccharic acid), application no. 17, preferably the coding region thereof, or a homolog or a fragment thereof, and being depicted in the same respective line as said Sll0418, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll0418, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll0418, or a functional equivalent or a homolog thereof as depicted in column 8 of Table II, application no. 17, preferably a homolog or functional equivalent as depicted in column 8 of Table II B, application no. 17, and being depicted in the same respective line as said Sll0418, and preferably the activity is increased non-targeted, whereby the respective line disclose in column 7 the fine chemical saccharic acid.

Accordingly, in one embodiment, the molecule which activity is to be increased in the process of the invention is the gene product with an activity as a “sll0418-protein”, preferably it is the molecule of section (a) or (b) of this paragraph.

In particular, it was observed that in plants, especially in Arabidopsis thaliana, increasing or generating the activity of a gene product non-targeted with the activity of a “sll0418-protein”, preferably being encoded by a gene comprising the nucleic acid sequence SEQ ID NO. 94942, preferably the coding region thereof, conferred the production of or the increase in saccharic acid compared with the wild type control.

The nucleic acid sequence of Sll0418 from Synechocystis sp., e.g. as shown in the respective line in column 5 of Table I, application no. 17, has been published in C. And the activity of the gene product thereof is the activity of sll0418-protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “sll0418-protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said Sll0418, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll0418, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll0418, or a functional equivalent or a homolog thereof as depicted in column 8 of Table II, application no. 17, preferably a homolog or functional equivalent as depicted in column 8 of Table II B, application no. 17, and being depicted in the same respective line as said Sll0418, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “sll0418-protein”, preferably it is the molecule of section (a) or (b) of this paragraph.

In particular, it was observed that in plants, especially in Arabidopsis thaliana, increasing or generating the activity of a gene product non-targeted with the activity of a “sll0418-protein”, preferably being encoded by a gene comprising the nucleic acid sequence SEQ ID NO. 94942, preferably the coding region thereof, conferred the production of or the increase in 3,4,5-trihydroxypentanoic acid compared with the wild type control.

The nucleic acid sequence of Sll0420 from Synechocystis sp., e.g. as shown in the respective line in column 5 of Table I, application no. 17, has been published in C. And the activity of the gene product thereof is the activity of urease subunit.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “urease subunit”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Sll0420, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll0420, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll0420, or a functional equivalent or a homolog thereof as depicted in column 8 of Table II, application no. 17, preferably a homolog or functional equivalent as depicted in column 8 of Table II B, application no. 17, and being depicted in the same respective line as said Sll0420, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “urease subunit”, preferably it is the molecule of section (a) or (b) of this paragraph.

In particular, it was observed that in plants, especially in Arabidopsis thaliana, increasing or generating the activity of a gene product non-targeted with the activity of a “urease subunit”, preferably being encoded by a gene comprising the nucleic acid sequence SEQ ID NO. 50104, preferably the coding region thereof, conferred the production of or the increase in glucose-1-phosphate compared with the wild type control.

The nucleic acid sequence of Sll0593 from Synechocystis sp., e.g. as shown in the respective line in column 5 of Table I, application no. 17, has been published in C. And the activity of the gene product thereof is the activity of glucokinase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “glucokinase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Sll0593, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll0593, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll0593, or a functional equivalent or a homolog thereof as depicted in column 8 of Table II, application no. 17, preferably a homolog or functional equivalent as depicted in column 8 of Table II B, application no. 17, and being depicted in the same respective line as said Sll0593, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “glucokinase”, preferably it is the molecule of section (a) or (b) of this paragraph.

In particular, it was observed that in plants, especially in Arabidopsis thaliana, increasing or generating the activity of a gene product plastidic with the activity of a “glucokinase”, preferably being encoded by a gene comprising the nucleic acid sequence SEQ ID NO. 85510, preferably the coding region thereof, conferred the production of or the increase in glucose-1-phosphate compared with the wild type control.

The nucleic acid sequence of Sll0643 from Synechocystis sp., e.g. as shown in the respective line in column 5 of Table I, application no. 17, has been published in C. And the activity of the gene product thereof is the activity of urease accessory protein.

Accordingly, in one embodiment, the process of the present invention for producing saccharic acid in a non-human organism, like a microorganism or a plant or a part thereof, comprises increasing or generating the activity of a gene product with the activity of a gene product conferring the activity of “urease accessory protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical saccharic acid), application no. 17, preferably the coding region thereof, or a homolog or a fragment thereof, and being depicted in the same respective line as said Sll0643, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll0643, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll0643, or a functional equivalent or a homolog thereof as depicted in column 8 of Table II, application no. 17, preferably a homolog or functional equivalent as depicted in column 8 of Table II B, application no. 17, and being depicted in the same respective line as said Sll0643, and preferably the activity is increased non-targeted, whereby the respective line disclose in column 7 the fine chemical saccharic acid.

Accordingly, in one embodiment, the molecule which activity is to be increased in the process of the invention is the gene product with an activity as a “urease accessory protein”, preferably it is the molecule of section (a) or (b) of this paragraph.

In particular, it was observed that in plants, especially in Arabidopsis thaliana, increasing or generating the activity of a gene product non-targeted with the activity of a “urease accessory protein”, preferably being encoded by a gene comprising the nucleic acid sequence SEQ ID NO. 146945, preferably the coding region thereof, conferred the production of or the increase in saccharic acid compared with the wild type control.

The nucleic acid sequence of Sll0833 from Synechocystis sp., e.g. as shown in the respective line in column 5 of Table I, application no. 17, has been published in C. And the activity of the gene product thereof is the activity of binding-protein-dependent transport systems inner membrane component.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “binding-protein-dependent transport systems inner membrane component”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Sll0833, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll0833, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll0833, or a functional equivalent or a homolog thereof as depicted in column 8 of Table II, application no. 17, preferably a homolog or functional equivalent as depicted in column 8 of Table II B, application no. 17, and being depicted in the same respective line as said Sll0833, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “binding-protein-dependent transport systems inner membrane component”, preferably it is the molecule of section (a) or (b) of this paragraph.

In particular, it was observed that in plants, especially in Arabidopsis thaliana, increasing or generating the activity of a gene product non-targeted with the activity of a “binding-protein-dependent transport systems inner membrane component”, preferably being encoded by a gene comprising the nucleic acid sequence SEQ ID NO. 101707, preferably the coding region thereof, conferred the production of or the increase in glucose-1-phosphate compared with the wild type control.

The nucleic acid sequence of Sll0833 from Synechocystis sp., e.g. as shown in the respective line in column 5 of Table I, application no. 17, has been published in C. And the activity of the gene product thereof is the activity of binding-protein-dependent transport systems inner membrane component.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “binding-protein-dependent transport systems inner membrane component”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said Sll0833, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll0833, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll0833, or a functional equivalent or a homolog thereof as depicted in column 8 of Table II, application no. 17, preferably a homolog or functional equivalent as depicted in column 8 of Table II B, application no. 17, and being depicted in the same respective line as said Sll0833, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “binding-protein-dependent transport systems inner membrane component”, preferably it is the molecule of section (a) or (b) of this paragraph.

In particular, it was observed that in plants, especially in Arabidopsis thaliana, increasing or generating the activity of a gene product non-targeted with the activity of a “binding-protein-dependent transport systems inner membrane component”, preferably being encoded by a gene comprising the nucleic acid sequence SEQ ID NO. 101707, preferably the coding region thereof, conferred the production of or the increase in 3,4,5-trihydroxypentanoic acid compared with the wild type control.

The nucleic acid sequence of Sll0934 from Synechocystis sp., e.g. as shown in the respective line in column 5 of Table I, application no. 17, has been published in C. And the activity of the gene product thereof is the activity of 3-deoxy-7-phosphoheptulonate synthase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “3-deoxy-7-phosphoheptulonate synthase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Sll0934, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll0934, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll0934, or a functional equivalent or a homolog thereof as depicted in column 8 of Table II, application no. 17, preferably a homolog or functional equivalent as depicted in column 8 of Table II B, application no. 17, and being depicted in the same respective line as said Sll0934, and preferably the activity is increased mitochondrial, whereby the respective line disclose in column 7 the fine chemical glucose-1-phosphate.

Accordingly, in one embodiment, the molecule which activity is to be increased in the process of the invention is the gene product with an activity as a “3-deoxy-7-phosphoheptulonate synthase”, preferably it is the molecule of section (a) or (b) of this paragraph.

In particular, it was observed that in plants, especially in Arabidopsis thaliana, increasing or generating the activity of a gene product mitochondrial with the activity of a “3-deoxy-7-phosphoheptulonate synthase”, preferably being encoded by a gene comprising the nucleic acid sequence SEQ ID NO. 52246, preferably the coding region thereof, conferred the production of or the increase in glucose-1-phosphate compared with the wild type control.

The nucleic acid sequence of Sll0945 from Synechocystis sp., e.g. as shown in the respective line in column 5 of Table I, application no. 17, has been published in C. And the activity of the gene product thereof is the activity of glycogen synthase.

Accordingly, in one embodiment, the process of the present invention for producing fructose in a non-human organism, like a microorganism or a plant or a part thereof, comprises increasing or generating the activity of a gene product with the activity of a gene product conferring the activity of “glycogen synthase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said Sll0945, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll0945, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll0945, or a functional equivalent or a homolog thereof as depicted in column 8 of Table II, application no. 17, preferably a homolog or functional equivalent as depicted in column 8 of Table II B, application no. 17, and being depicted in the same respective line as said Sll0945, and preferably the activity is increased non-targeted, whereby the respective line disclose in column 7 the fine chemical fructose.

Accordingly, in one embodiment, the molecule which activity is to be increased in the process of the invention is the gene product with an activity as a “glycogen synthase”, preferably it is the molecule of section (a) or (b) of this paragraph.

In particular, it was observed that in plants, especially in Arabidopsis thaliana, increasing or generating the activity of a gene product non-targeted with the activity of a “glycogen synthase”, preferably being encoded by a gene comprising the nucleic acid sequence SEQ ID NO. 52364, preferably the coding region thereof, conferred the production of or the increase in fructose compared with the wild type control.

The nucleic acid sequence of Sll0945 from Synechocystis sp., e.g. as shown in the respective line in column 5 of Table I, application no. 17, has been published in C. And the activity of the gene product thereof is the activity of glycogen synthase.

Accordingly, in one embodiment, the process of the present invention for producing sucrose in a non-human organism, like a microorganism or a plant or a part thereof, comprises increasing or generating the activity of a gene product with the activity of a gene product conferring the activity of “glycogen synthase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said Sll0945, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll0945, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll0945, or a functional equivalent or a homolog thereof as depicted in column 8 of Table II, application no. 17, preferably a homolog or functional equivalent as depicted in column 8 of Table II B, application no. 17, and being depicted in the same respective line as said Sll0945, and preferably the activity is increased non-targeted, whereby the respective line disclose in column 7 the fine chemical sucrose.

Accordingly, in one embodiment, the molecule which activity is to be increased in the process of the invention is the gene product with an activity as a “glycogen synthase”, preferably it is the molecule of section (a) or (b) of this paragraph.

In particular, it was observed that in plants, especially in Arabidopsis thaliana, increasing or generating the activity of a gene product non-targeted with the activity of a “glycogen synthase”, preferably being encoded by a gene comprising the nucleic acid sequence SEQ ID NO. 52364, preferably the coding region thereof, conferred the production of or the increase in sucrose compared with the wild type control.

The nucleic acid sequence of Sll1185 from Synechocystis sp., e.g. as shown in the respective line in column 5 of Table I, application no. 17, has been published in C. And the activity of the gene product thereof is the activity of coproporphyrinogen oxidase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “coproporphyrinogen oxidase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Sll1185, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll1185, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll1185, or a functional equivalent or a homolog thereof as depicted in column 8 of Table II, application no. 17, preferably a homolog or functional equivalent as depicted in column 8 of Table II B, application no. 17, and being depicted in the same respective line as said Sll1185, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “coproporphyrinogen oxidase”, preferably it is the molecule of section (a) or (b) of this paragraph.

In particular, it was observed that in plants, especially in Arabidopsis thaliana, increasing or generating the activity of a gene product non-targeted with the activity of a “coproporphyrinogen oxidase”, preferably being encoded by a gene comprising the nucleic acid sequence SEQ ID NO. 53189, preferably the coding region thereof, conferred the production of or the increase in glucose-1-phosphate compared with the wild type control.

The nucleic acid sequence of SLL1308 from Synechocystis sp., e.g. as shown in the respective line in column 5 of Table I, application no. 17, has been published in C. And the activity of the gene product thereof is the activity of short chain dehydrogenase.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “short chain dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said SLL1308, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said SLL1308, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said SLL1308, or a functional equivalent or a homolog thereof as depicted in column 8 of Table II, application no. 17, preferably a homolog or functional equivalent as depicted in column 8 of Table II B, application no. 17, and being depicted in the same respective line as said SLL1308, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “short chain dehydrogenase”, preferably it is the molecule of section (a) or (b) of this paragraph.

In particular, it was observed that in plants, especially in Arabidopsis thaliana, increasing or generating the activity of a gene product non-targeted with the activity of a “short chain dehydrogenase”, preferably being encoded by a gene comprising the nucleic acid sequence SEQ ID NO. 147208, preferably the coding region thereof, conferred the production of or the increase in 3,4,5-trihydroxypentanoic acid compared with the wild type control.

The nucleic acid sequence of Sll1393 from Synechocystis sp., e.g. as shown in the respective line in column 5 of Table I, application no. 17, has been published in C. And the activity of the gene product thereof is the activity of glycogen (starch) synthase.

Accordingly, in one embodiment, the process of the present invention for producing raffinose in a non-human organism, like a microorganism or a plant or a part thereof, comprises increasing or generating the activity of a gene product with the activity of a gene product conferring the activity of “glycogen (starch) synthase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said Sll1393, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll1393, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll1393, or a functional equivalent or a homolog thereof as depicted in column 8 of Table II, application no. 17, preferably a homolog or functional equivalent as depicted in column 8 of Table II B, application no. 17, and being depicted in the same respective line as said Sll1393, and preferably the activity is increased non-targeted, whereby the respective line disclose in column 7 the fine chemical raffinose.

Accordingly, in one embodiment, the molecule which activity is to be increased in the process of the invention is the gene product with an activity as a “glycogen (starch) synthase”, preferably it is the molecule of section (a) or (b) of this paragraph.

In particular, it was observed that in plants, especially in Arabidopsis thaliana, increasing or generating the activity of a gene product non-targeted with the activity of a “glycogen (starch) synthase”, preferably being encoded by a gene comprising the nucleic acid sequence SEQ ID NO. 53456, preferably the coding region thereof, conferred the production of or the increase in raffinose compared with the wild type control.

The nucleic acid sequence of Sll1498 from Synechocystis sp., e.g. as shown in the respective line in column 5 of Table I, application no. 17, has been published in C. And the activity of the gene product thereof is the activity of carbamoyl-phosphate synthase subunit.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “carbamoyl-phosphate synthase subunit”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said Sll1498, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll1498, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll1498, or a functional equivalent or a homolog thereof as depicted in column 8 of Table II, application no. 17, preferably a homolog or functional equivalent as depicted in column 8 of Table II B, application no. 17, and being depicted in the same respective line as said Sll1498, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “carbamoyl-phosphate synthase subunit”, preferably it is the molecule of section (a) or (b) of this paragraph.

In particular, it was observed that in plants, especially in Arabidopsis thaliana, increasing or generating the activity of a gene product non-targeted with the activity of a “carbamoylphosphate synthase subunit”, preferably being encoded by a gene comprising the nucleic acid sequence SEQ ID NO. 95047, preferably the coding region thereof, conferred the production of or the increase in myo-inositol compared with the wild type control.

The nucleic acid sequence of Sll1498 from Synechocystis sp., e.g. as shown in the respective line in column 5 of Table I, application no. 17, has been published in C. And the activity of the gene product thereof is the activity of carbamoyl-phosphate synthase subunit.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “carbamoyl-phosphate synthase subunit”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said Sll1498, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll1498, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll1498, or a functional equivalent or a homolog thereof as depicted in column 8 of Table II, application no. 17, preferably a homolog or functional equivalent as depicted in column 8 of Table II B, application no. 17, and being depicted in the same respective line as said Sll1498, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “carbamoyl-phosphate synthase subunit”, preferably it is the molecule of section (a) or (b) of this paragraph.

In particular, it was observed that in plants, especially in Arabidopsis thaliana, increasing or generating the activity of a gene product non-targeted with the activity of a “carbamoylphosphate synthase subunit”, preferably being encoded by a gene comprising the nucleic acid sequence SEQ ID NO. 95047, preferably the coding region thereof, conferred the production of or the increase in 3,4,5-trihydroxypentanoic acid compared with the wild type control.

The nucleic acid sequence of Sll1676 from Synechocystis sp., e.g. as shown in the respective line in column 5 of Table I, application no. 17, has been published in C. And the activity of the gene product thereof is the activity of 4-alpha-glucanotransferase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “4-alpha-glucanotransferase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said Sll1676, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll1676, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll1676, or a functional equivalent or a homolog thereof as depicted in column 8 of Table II, application no. 17, preferably a homolog or functional equivalent as depicted in column 8 of Table II B, application no. 17, and being depicted in the same respective line as said Sll1676, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “4-alpha-glucanotransferase”, preferably it is the molecule of section (a) or (b) of this paragraph.

In particular, it was observed that in plants, especially in Arabidopsis thaliana, increasing or generating the activity of a gene product non-targeted with the activity of a “4-alpha-glucanotransferase”, preferably being encoded by a gene comprising the nucleic acid sequence SEQ ID NO. 54897, preferably the coding region thereof, conferred the production of or the increase in myo-inositol compared with the wild type control.

The nucleic acid sequence of Sll1682 from Synechocystis sp., e.g. as shown in the respective line in column 5 of Table I, application no. 17, has been published in C. And the activity of the gene product thereof is the activity of alanine dehydrogenase.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “alanine dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said Sll1682, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll1682, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll1682, or a functional equivalent or a homolog thereof as depicted in column 8 of Table II, application no. 17, preferably a homolog or functional equivalent as depicted in column 8 of Table II B, application no. 17, and being depicted in the same respective line as said Sll1682, and preferably the activity is increased plastidic, whereby the respective line disclose in column 7 the fine chemical 3,4,5-trihydroxypentanoic acid.

Accordingly, in one embodiment, the molecule which activity is to be increased in the process of the invention is the gene product with an activity as a “alanine dehydrogenase”, preferably it is the molecule of section (a) or (b) of this paragraph.

In particular, it was observed that in plants, especially in Arabidopsis thaliana, increasing or generating the activity of a gene product plastidic with the activity of a “alanine dehydrogenase”, preferably being encoded by a gene comprising the nucleic acid sequence SEQ ID NO. 55063, preferably the coding region thereof, conferred the production of or the increase in 3,4,5-trihydroxypentanoic acid compared with the wild type control.

The nucleic acid sequence of Sll1761 from Synechocystis sp., e.g. as shown in the respective line in column 5 of Table I, application no. 17, has been published in C. And the activity of the gene product thereof is the activity of sll1761-protein.

Accordingly, in one embodiment, the process of the present invention for producing glucose in a non-human organism, like a microorganism or a plant or a part thereof, comprises increasing or generating the activity of a gene product with the activity of a gene product conferring the activity of “sll1761-protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said Sll1761, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll1761, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll1761, or a functional equivalent or a homolog thereof as depicted in column 8 of Table II, application no. 17, preferably a homolog or functional equivalent as depicted in column 8 of Table II B, application no. 17, and being depicted in the same respective line as said Sll1761, and preferably the activity is increased non-targeted, whereby the respective line disclose in column 7 the fine chemical glucose.

Accordingly, in one embodiment, the molecule which activity is to be increased in the process of the invention is the gene product with an activity as a “sll1761-protein”, preferably it is the molecule of section (a) or (b) of this paragraph.

In particular, it was observed that in plants, especially in Arabidopsis thaliana, increasing or generating the activity of a gene product non-targeted with the activity of a “sll1761-protein”, preferably being encoded by a gene comprising the nucleic acid sequence SEQ ID NO. 55379, preferably the coding region thereof, conferred the production of or the increase in glucose compared with the wild type control.

The nucleic acid sequence of Sll1848 from Synechocystis sp., e.g. as shown in the respective line in column 5 of Table I, application no. 17, has been published in C. And the activity of the gene product thereof is the activity of acyl transferase.

Accordingly, in one embodiment, the process of the present invention for producing glucose in a non-human organism, like a microorganism or a plant or a part thereof, comprises increasing or generating the activity of a gene product with the activity of a gene product conferring the activity of “acyl transferase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said Sll1848, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll1848, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll1848, or a functional equivalent or a homolog thereof as depicted in column 8 of Table II, application no. 17, preferably a homolog or functional equivalent as depicted in column 8 of Table II B, application no. 17, and being depicted in the same respective line as said Sll1848, and preferably the activity is increased plastidic, whereby the respective line disclose in column 7 the fine chemical glucose.

Accordingly, in one embodiment, the molecule which activity is to be increased in the process of the invention is the gene product with an activity as a “acyl transferase”, preferably it is the molecule of section (a) or (b) of this paragraph.

In particular, it was observed that in plants, especially in Arabidopsis thaliana, increasing or generating the activity of a gene product plastidic with the activity of a “acyl transferase”, preferably being encoded by a gene comprising the nucleic acid sequence SEQ ID NO. 86447, preferably the coding region thereof, conferred the production of or the increase in glucose compared with the wild type control.

The nucleic acid sequence of Sll1920 from Synechocystis sp., e.g. as shown in the respective line in column 5 of Table I, application no. 17, has been published in C. And the activity of the gene product thereof is the activity of cation-transporting ATPase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “cation-transporting ATPase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Sll1920, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll1920, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll1920, or a functional equivalent or a homolog thereof as depicted in column 8 of Table II, application no. 17, preferably a homolog or functional equivalent as depicted in column 8 of Table II B, application no. 17, and being depicted in the same respective line as said Sll1920, and preferably the activity is increased mitochondrial, whereby the respective line disclose in column 7 the fine chemical glucose-1-phosphate.

Accordingly, in one embodiment, the molecule which activity is to be increased in the process of the invention is the gene product with an activity as a “cation-transporting ATPase”, preferably it is the molecule of section (a) or (b) of this paragraph.

In particular, it was observed that in plants, especially in Arabidopsis thaliana, increasing or generating the activity of a gene product mitochondrial with the activity of a “cation-transporting ATPase”, preferably being encoded by a gene comprising the nucleic acid sequence SEQ ID NO. 55385, preferably the coding region thereof, conferred the production of or the increase in glucose-1-phosphate compared with the wild type control.

The nucleic acid sequence of Sll1920 from Synechocystis sp., e.g. as shown in the respective line in column 5 of Table I, application no. 17, has been published in C. And the activity of the gene product thereof is the activity of cation-transporting ATPase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “cation-transporting ATPase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Sll1920, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll1920, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll1920, or a functional equivalent or a homolog thereof as depicted in column 8 of Table II, application no. 17, preferably a homolog or functional equivalent as depicted in column 8 of Table II B, application no. 17, and being depicted in the same respective line as said Sll1920, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “cation-transporting ATPase”, preferably it is the molecule of section (a) or (b) of this paragraph.

In particular, it was observed that in plants, especially in Arabidopsis thaliana, increasing or generating the activity of a gene product non-targeted with the activity of a “cation-transporting ATPase”, preferably being encoded by a gene comprising the nucleic acid sequence SEQ ID NO. 55385, preferably the coding region thereof, conferred the production of or the increase in glucose-1-phosphate compared with the wild type control.

The nucleic acid sequence of Sll1920 from Synechocystis sp., e.g. as shown in the respective line in column 5 of Table I, application no. 17, has been published in C. And the activity of the gene product thereof is the activity of cation-transporting ATPase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “cation-transporting ATPase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said Sll1920, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll1920, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll1920, or a functional equivalent or a homolog thereof as depicted in column 8 of Table II, application no. 17, preferably a homolog or functional equivalent as depicted in column 8 of Table II B, application no. 17, and being depicted in the same respective line as said Sll1920, and preferably the activity is increased mitochondrial, whereby the respective line disclose in column 7 the fine chemical myo-inositol.

Accordingly, in one embodiment, the molecule which activity is to be increased in the process of the invention is the gene product with an activity as a “cation-transporting ATPase”, preferably it is the molecule of section (a) or (b) of this paragraph.

In particular, it was observed that in plants, especially in Arabidopsis thaliana, increasing or generating the activity of a gene product mitochondrial with the activity of a “cation-transporting ATPase”, preferably being encoded by a gene comprising the nucleic acid sequence SEQ ID NO. 55385, preferably the coding region thereof, conferred the production of or the increase in myo-inositol compared with the wild type control.

The nucleic acid sequence of Sll1920 from Synechocystis sp., e.g. as shown in the respective line in column 5 of Table I, application no. 17, has been published in C. And the activity of the gene product thereof is the activity of cation-transporting ATPase.

Accordingly, in one embodiment, the process of the present invention for producing raffinose in a non-human organism, like a microorganism or a plant or a part thereof, comprises increasing or generating the activity of a gene product with the activity of a gene product conferring the activity of “cation-transporting ATPase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said Sll1920, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Sll1920, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Sll1920, or a functional equivalent or a homolog thereof as depicted in column 8 of Table II, application no. 17, preferably a homolog or functional equivalent as depicted in column 8 of Table II B, application no. 17, and being depicted in the same respective line as said Sll1920, and preferably the activity is increased non-targeted, whereby the respective line disclose in column 7 the fine chemical raffinose.

Accordingly, in one embodiment, the molecule which activity is to be increased in the process of the invention is the gene product with an activity as a “cation-transporting ATPase”, preferably it is the molecule of section (a) or (b) of this paragraph.

In particular, it was observed that in plants, especially in Arabidopsis thaliana, increasing or generating the activity of a gene product non-targeted with the activity of a “cation-transporting ATPase”, preferably being encoded by a gene comprising the nucleic acid sequence SEQ ID NO. 55385, preferably the coding region thereof, conferred the production of or the increase in raffinose compared with the wild type control.

The nucleic acid sequence of Slr0237 from Synechocystis sp., e.g. as shown in the respective line in column 5 of Table I, application no. 17, has been published in C. And the activity of the gene product thereof is the activity of glycosidase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “glycosidase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Slr0237, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr0237, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr0237, or a functional equivalent or a homolog thereof as depicted in column 8 of Table II, application no. 17, preferably a homolog or functional equivalent as depicted in column 8 of Table II B, application no. 17, and being depicted in the same respective line as said Slr0237, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “glycosidase”, preferably it is the molecule of section (a) or (b) of this paragraph.

In particular, it was observed that in plants, especially in Arabidopsis thaliana, increasing or generating the activity of a gene product non-targeted with the activity of a “glycosidase”, preferably being encoded by a gene comprising the nucleic acid sequence SEQ ID NO. 55771, preferably the coding region thereof, conferred the production of or the increase in glucose-1-phosphate compared with the wild type control.

The nucleic acid sequence of Slr0239 from Synechocystis sp., e.g. as shown in the respective line in column 5 of Table I, application no. 17, has been published in C. And the activity of the gene product thereof is the activity of precorrin methylase.

Accordingly, in one embodiment, the process of the present invention for producing fructose in a non-human organism, like a microorganism or a plant or a part thereof, comprises increasing or generating the activity of a gene product with the activity of a gene product conferring the activity of “precorrin methylase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said Slr0239, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr0239, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr0239, or a functional equivalent or a homolog thereof as depicted in column 8 of Table II, application no. 17, preferably a homolog or functional equivalent as depicted in column 8 of Table II B, application no. 17, and being depicted in the same respective line as said Slr0239, and preferably the activity is increased mitochondrial, whereby the respective line disclose in column 7 the fine chemical fructose.

Accordingly, in one embodiment, the molecule which activity is to be increased in the process of the invention is the gene product with an activity as a “precorrin methylase”, preferably it is the molecule of section (a) or (b) of this paragraph.

In particular, it was observed that in plants, especially in Arabidopsis thaliana, increasing or generating the activity of a gene product mitochondrial with the activity of a “precorrin methylase”, preferably being encoded by a gene comprising the nucleic acid sequence SEQ ID NO. 86540, preferably the coding region thereof, conferred the production of or the increase in fructose compared with the wild type control.

The nucleic acid sequence of Slr0239 from Synechocystis sp., e.g. as shown in the respective line in column 5 of Table I, application no. 17, has been published in C. And the activity of the gene product thereof is the activity of precorrin methylase.

Accordingly, in one embodiment, the process of the present invention for producing glucose in a non-human organism, like a microorganism or a plant or a part thereof, comprises increasing or generating the activity of a gene product with the activity of a gene product conferring the activity of “precorrin methylase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said Slr0239, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr0239, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr0239, or a functional equivalent or a homolog thereof as depicted in column 8 of Table II, application no. 17, preferably a homolog or functional equivalent as depicted in column 8 of Table II B, application no. 17, and being depicted in the same respective line as said Slr0239, and preferably the activity is increased mitochondrial, whereby the respective line disclose in column 7 the fine chemical glucose.

Accordingly, in one embodiment, the molecule which activity is to be increased in the process of the invention is the gene product with an activity as a “precorrin methylase”, preferably it is the molecule of section (a) or (b) of this paragraph.

In particular, it was observed that in plants, especially in Arabidopsis thaliana, increasing or generating the activity of a gene product mitochondrial with the activity of a “precorrin methylase”, preferably being encoded by a gene comprising the nucleic acid sequence SEQ ID NO. 86540, preferably the coding region thereof, conferred the production of or the increase in glucose compared with the wild type control.

The nucleic acid sequence of Slr0338 from Synechocystis sp., e.g. as shown in the respective line in column 5 of Table I, application no. 17, has been published in C. And the activity of the gene product thereof is the activity of oxidoreductase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “oxidoreductase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Slr0338, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr0338, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr0338, or a functional equivalent or a homolog thereof as depicted in column 8 of Table II, application no. 17, preferably a homolog or functional equivalent as depicted in column 8 of Table II B, application no. 17, and being depicted in the same respective line as said Slr0338, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 11990, preferably the coding region thereof, conferred the production of or the increase in glucose-1-phosphate compared with the wild type control.

The nucleic acid sequence of Slr0338 from Synechocystis sp., e.g. as shown in the respective line in column 5 of Table I, application no. 17, has been published in C. And the activity of the gene product thereof is the activity of oxidoreductase.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “oxidoreductase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said Slr0338, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr0338, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr0338, or a functional equivalent or a homolog thereof as depicted in column 8 of Table II, application no. 17, preferably a homolog or functional equivalent as depicted in column 8 of Table II B, application no. 17, and being depicted in the same respective line as said Slr0338, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 11990, preferably the coding region thereof, conferred the production of or the increase in 3,4,5-trihydroxypentanoic acid compared with the wild type control.

The nucleic acid sequence of Slr0600 from Synechocystis sp., e.g. as shown in the respective line in column 5 of Table I, application no. 17, has been published in C. And the activity of the gene product thereof is the activity of slr0600-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “slr0600-protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Slr0600, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr0600, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr0600, or a functional equivalent or a homolog thereof as depicted in column 8 of Table II, application no. 17, preferably a homolog or functional equivalent as depicted in column 8 of Table II B, application no. 17, and being depicted in the same respective line as said Slr0600, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “slr0600-protein”, preferably it is the molecule of section (a) or (b) of this paragraph.

In particular, it was observed that in plants, especially in Arabidopsis thaliana, increasing or generating the activity of a gene product non-targeted with the activity of a “slr0600-protein”, preferably being encoded by a gene comprising the nucleic acid sequence SEQ ID NO. 56514, preferably the coding region thereof, conferred the production of or the increase in glucose-1-phosphate compared with the wild type control.

The nucleic acid sequence of Slr0710 from Synechocystis sp., e.g. as shown in the respective line in column 5 of Table I, application no. 17, has been published in C. And the activity of the gene product thereof is the activity of Glu/Leu/Phe/Val dehydrogenase .

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “Glu/Leu/Phe/Val dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Slr0710, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr0710, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr0710, or a functional equivalent or a homolog thereof as depicted in column 8 of Table II, application no. 17, preferably a homolog or functional equivalent as depicted in column 8 of Table II B, application no. 17, and being depicted in the same respective line as said Slr0710, and preferably the activity is increased mitochondrial, whereby the respective line disclose in column 7 the fine chemical glucose-1-phosphate.

Accordingly, in one embodiment, the molecule which activity is to be increased in the process of the invention is the gene product with an activity as a “Glu/Leu/Phe/Val dehydrogenase”, preferably it is the molecule of section (a) or (b) of this paragraph.

In particular, it was observed that in plants, especially in Arabidopsis thaliana, increasing or generating the activity of a gene product mitochondrial with the activity of a “Glu/Leu/PheNal dehydrogenase”, preferably being encoded by a gene comprising the nucleic acid sequence SEQ ID NO. 56894, preferably the coding region thereof, conferred the production of or the increase in glucose-1-phosphate compared with the wild type control.

The nucleic acid sequence of Slr0721 from Synechocystis sp., e.g. as shown in the respective line in column 5 of Table I, application no. 17, has been published in C. And the activity of the gene product thereof is the activity of malate dehydrogenase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “malate dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Slr0721, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr0721, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr0721, or a functional equivalent or a homolog thereof as depicted in column 8 of Table II, application no. 17, preferably a homolog or functional equivalent as depicted in column 8 of Table II B, application no. 17, and being depicted in the same respective line as said Slr0721, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “malate dehydrogenase”, preferably it is the molecule of section (a) or (b) of this paragraph.

In particular, it was observed that in plants, especially in Arabidopsis thaliana, increasing or generating the activity of a gene product plastidic with the activity of a “malate dehydrogenase”, preferably being encoded by a gene comprising the nucleic acid sequence SEQ ID NO. 87397, preferably the coding region thereof, conferred the production of or the increase in glucose-1-phosphate compared with the wild type control.

The nucleic acid sequence of Slr0739 from Synechocystis sp., e.g. as shown in the respective line in column 5 of Table I, application no. 17, has been published in C. And the activity of the gene product thereof is the activity of geranylgeranyl pyrophosphate synthase.

Accordingly, in one embodiment, the process of the present invention for producing sucrose in a non-human organism, like a microorganism or a plant or a part thereof, comprises increasing or generating the activity of a gene product with the activity of a gene product conferring the activity of “geranylgeranyl pyrophosphate synthase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said Slr0739, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr0739, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr0739, or a functional equivalent or a homolog thereof as depicted in column 8 of Table II, application no. 17, preferably a homolog or functional equivalent as depicted in column 8 of Table II B, application no. 17, and being depicted in the same respective line as said Slr0739, and preferably the activity is increased plastidic, whereby the respective line disclose in column 7 the fine chemical sucrose.

Accordingly, in one embodiment, the molecule which activity is to be increased in the process of the invention is the gene product with an activity as a “geranylgeranyl pyrophosphate synthase”, preferably it is the molecule of section (a) or (b) of this paragraph.

In particular, it was observed that in plants, especially in Arabidopsis thaliana, increasing or generating the activity of a gene product plastidic with the activity of a “geranylgeranyl pyrophosphate synthase”, preferably being encoded by a gene comprising the nucleic acid sequence SEQ ID NO. 57235, preferably the coding region thereof, conferred the production of or the increase in sucrose compared with the wild type control.

The nucleic acid sequence of Slr0757 from Synechocystis sp., e.g. as shown in the respective line in column 5 of Table I, application no. 17, has been published in C. And the activity of the gene product thereof is the activity of circadian clock protein.

Accordingly, in one embodiment, the process of the present invention for producing fructose in a non-human organism, like a microorganism or a plant or a part thereof, comprises increasing or generating the activity of a gene product with the activity of a gene product conferring the activity of “circadian clock protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said Slr0757, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr0757, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr0757, or a functional equivalent or a homolog thereof as depicted in column 8 of Table II, application no. 17, preferably a homolog or functional equivalent as depicted in column 8 of Table II B, application no. 17, and being depicted in the same respective line as said Slr0757, and preferably the activity is increased mitochondrial, whereby the respective line disclose in column 7 the fine chemical fructose.

Accordingly, in one embodiment, the molecule which activity is to be increased in the process of the invention is the gene product with an activity as a “circadian clock protein”, preferably it is the molecule of section (a) or (b) of this paragraph.

In particular, it was observed that in plants, especially in Arabidopsis thaliana, increasing or generating the activity of a gene product mitochondrial with the activity of a “circadian clock protein”, preferably being encoded by a gene comprising the nucleic acid sequence SEQ ID NO. 147346, preferably the coding region thereof, conferred the production of or the increase in fructose compared with the wild type control.

The nucleic acid sequence of Slr0757 from Synechocystis sp., e.g. as shown in the respective line in column 5 of Table I, application no. 17, has been published in C. And the activity of the gene product thereof is the activity of circadian clock protein.

Accordingly, in one embodiment, the process of the present invention for producing glucose in a non-human organism, like a microorganism or a plant or a part thereof, comprises increasing or generating the activity of a gene product with the activity of a gene product conferring the activity of “circadian clock protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said Slr0757, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr0757, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr0757, or a functional equivalent or a homolog thereof as depicted in column 8 of Table II, application no. 17, preferably a homolog or functional equivalent as depicted in column 8 of Table II B, application no. 17, and being depicted in the same respective line as said Slr0757, and preferably the activity is increased mitochondrial, whereby the respective line disclose in column 7 the fine chemical glucose.

Accordingly, in one embodiment, the molecule which activity is to be increased in the process of the invention is the gene product with an activity as a “circadian clock protein”, preferably it is the molecule of section (a) or (b) of this paragraph.

In particular, it was observed that in plants, especially in Arabidopsis thaliana, increasing or generating the activity of a gene product mitochondrial with the activity of a “circadian clock protein”, preferably being encoded by a gene comprising the nucleic acid sequence SEQ ID NO. 147346, preferably the coding region thereof, conferred the production of or the increase in glucose compared with the wild type control.

The nucleic acid sequence of Slr0757 from Synechocystis sp., e.g. as shown in the respective line in column 5 of Table I, application no. 17, has been published in C. And the activity of the gene product thereof is the activity of circadian clock protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “circadian clock protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Slr0757, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr0757, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr0757, or a functional equivalent or a homolog thereof as depicted in column 8 of Table II, application no. 17, preferably a homolog or functional equivalent as depicted in column 8 of Table II B, application no. 17, and being depicted in the same respective line as said Slr0757, and preferably the activity is increased mitochondrial, whereby the respective line disclose in column 7 the fine chemical glucose-1-phosphate.

Accordingly, in one embodiment, the molecule which activity is to be increased in the process of the invention is the gene product with an activity as a “circadian clock protein”, preferably it is the molecule of section (a) or (b) of this paragraph.

In particular, it was observed that in plants, especially in Arabidopsis thaliana, increasing or generating the activity of a gene product mitochondrial with the activity of a “circadian clock protein”, preferably being encoded by a gene comprising the nucleic acid sequence SEQ ID NO. 147346, preferably the coding region thereof, conferred the production of or the increase in glucose-1-phosphate compared with the wild type control.

The nucleic acid sequence of Slr0782 from Synechocystis sp., e.g. as shown in the respective line in column 5 of Table I, application no. 17, has been published in C. And the activity of the gene product thereof is the activity of amine oxidase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “amine oxidase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Slr0782, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr0782, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr0782, or a functional equivalent or a homolog thereof as depicted in column 8 of Table II, application no. 17, preferably a homolog or functional equivalent as depicted in column 8 of Table II B, application no. 17, and being depicted in the same respective line as said Slr0782, and preferably the activity is increased mitochondrial, whereby the respective line disclose in column 7 the fine chemical glucose-1-phosphate.

Accordingly, in one embodiment, the molecule which activity is to be increased in the process of the invention is the gene product with an activity as a “amine oxidase”, preferably it is the molecule of section (a) or (b) of this paragraph.

In particular, it was observed that in plants, especially in Arabidopsis thaliana, increasing or generating the activity of a gene product mitochondrial with the activity of a “amine oxidase”, preferably being encoded by a gene comprising the nucleic acid sequence SEQ ID NO. 57679, preferably the coding region thereof, conferred the production of or the increase in glucose-1-phosphate compared with the wild type control.

The nucleic acid sequence of Slr0862 from Synechocystis sp., e.g. as shown in the respective line in column 5 of Table I, application no. 17, has been published in C. And the activity of the gene product thereof is the activity of kinase.

Accordingly, in one embodiment, the process of the present invention for producing fructose in a non-human organism, like a microorganism or a plant or a part thereof, comprises increasing or generating the activity of a gene product with the activity of a gene product conferring the activity of “kinase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said Slr0862, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr0862, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr0862, or a functional equivalent or a homolog thereof as depicted in column 8 of Table II, application no. 17, preferably a homolog or functional equivalent as depicted in column 8 of Table II B, application no. 17, and being depicted in the same respective line as said Slr0862, and preferably the activity is increased mitochondrial, whereby the respective line disclose in column 7 the fine chemical fructose.

Accordingly, in one embodiment, the molecule which activity is to be increased in the process of the invention is the gene product with an activity as a “kinase”, preferably it is the molecule of section (a) or (b) of this paragraph.

In particular, it was observed that in plants, especially in Arabidopsis thaliana, increasing or generating the activity of a gene product mitochondrial with the activity of a “kinase”, preferably being encoded by a gene comprising the nucleic acid sequence SEQ ID NO. 87610, preferably the coding region thereof, conferred the production of or the increase in fructose compared with the wild type control.

The nucleic acid sequence of Slr0862 from Synechocystis sp., e.g. as shown in the respective line in column 5 of Table I, application no. 17, has been published in C. And the activity of the gene product thereof is the activity of kinase.

Accordingly, in one embodiment, the process of the present invention for producing glucose in a non-human organism, like a microorganism or a plant or a part thereof, comprises increasing or generating the activity of a gene product with the activity of a gene product conferring the activity of “kinase”, especially from Synechocystis sp. or its functional equivalent or its homolog, e.g.

the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said Slr0862, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr0862, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr0862, or a functional equivalent or a homolog thereof as depicted in column 8 of Table II, application no. 17, preferably a homolog or functional equivalent as depicted in column 8 of Table II B, application no. 17, and being depicted in the same respective line as said Slr0862, and preferably the activity is increased mitochondrial, whereby the respective line disclose in column 7 the fine chemical glucose.

Accordingly, in one embodiment, the molecule which activity is to be increased in the process of the invention is the gene product with an activity as a “kinase”, preferably it is the molecule of section (a) or (b) of this paragraph.

In particular, it was observed that in plants, especially in Arabidopsis thaliana, increasing or generating the activity of a gene product mitochondrial with the activity of a “kinase”, preferably being encoded by a gene comprising the nucleic acid sequence SEQ ID NO. 87610, preferably the coding region thereof, conferred the production of or the increase in glucose compared with the wild type control.

The nucleic acid sequence of Slr0905 from Synechocystis sp., e.g. as shown in the respective line in column 5 of Table I, application no. 17, has been published in C. And the activity of the gene product thereof is the activity of Mg-protoporphyrin IX monomethyl ester oxidative cyclase 66 kD subunit.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “Mg-protoporphyrin IX monomethyl ester oxidative cyclase 66 kD subunit”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Slr0905, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr0905, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr0905, or a functional equivalent or a homolog thereof as depicted in column 8 of Table II, application no. 17, preferably a homolog or functional equivalent as depicted in column 8 of Table II B, application no. 17, and being depicted in the same respective line as said Slr0905, and preferably the activity is increased mitochondrial, whereby the respective line disclose in column 7 the fine chemical glucose-1-phosphate.

Accordingly, in one embodiment, the molecule which activity is to be increased in the process of the invention is the gene product with an activity as a “Mg-protoporphyrin IX monomethyl ester oxidative cyclase 66 kD subunit”, preferably it is the molecule of section (a) or (b) of this paragraph.

In particular, it was observed that in plants, especially in Arabidopsis thaliana, increasing or generating the activity of a gene product mitochondrial with the activity of a “Mg-protoporphyrin IX monomethyl ester oxidative cyclase 66 kD subunit”, preferably being encoded by a gene comprising the nucleic acid sequence SEQ ID NO. 147407, preferably the coding region thereof, conferred the production of or the increase in glucose-1-phosphate compared with the wild type control.

The nucleic acid sequence of Slr0949 from Synechocystis sp., e.g. as shown in the respective line in column 5 of Table I, application no. 17, has been published in C. And the activity of the gene product thereof is the activity of permease protein of ABC transporter.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “permease protein of ABC transporter”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said Slr0949, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr0949, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr0949, or a functional equivalent or a homolog thereof as depicted in column 8 of Table II, application no. 17, preferably a homolog or functional equivalent as depicted in column 8 of Table II B, application no. 17, and being depicted in the same respective line as said Slr0949, and preferably the activity is increased mitochondrial, whereby the respective line disclose in column 7 the fine chemical myo-inositol.

Accordingly, in one embodiment, the molecule which activity is to be increased in the process of the invention is the gene product with an activity as a “permease protein of ABC transporter”, preferably it is the molecule of section (a) or (b) of this paragraph.

In particular, it was observed that in plants, especially in Arabidopsis thaliana, increasing or generating the activity of a gene product mitochondrial with the activity of a “permease protein of ABC transporter”, preferably being encoded by a gene comprising the nucleic acid sequence SEQ ID NO. 119222, preferably the coding region thereof, conferred the production of or the increase in myo-inositol compared with the wild type control.

The nucleic acid sequence of Slr1096 from Synechocystis sp., e.g. as shown in the respective line in column 5 of Table I, application no. 17, has been published in C. And the activity of the gene product thereof is the activity of dihydrolipoamide dehydrogenase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “dihydrolipoamide dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Slr1096, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr1096, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr1096, or a functional equivalent or a homolog thereof as depicted in column 8 of Table II, application no. 17, preferably a homolog or functional equivalent as depicted in column 8 of Table II B, application no. 17, and being depicted in the same respective line as said Slr1096, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “dihydrolipoamide dehydrogenase”, preferably it is the molecule of section (a) or (b) of this paragraph.

In particular, it was observed that in plants, especially in Arabidopsis thaliana, increasing or generating the activity of a gene product non-targeted with the activity of a “dihydrolipoamide dehydrogenase”, preferably being encoded by a gene comprising the nucleic acid sequence SEQ ID NO. 57734, preferably the coding region thereof, conferred the production of or the increase in glucose-1-phosphate compared with the wild type control.

The nucleic acid sequence of Slr1096 from Synechocystis sp., e.g. as shown in the respective line in column 5 of Table I, application no. 17, has been published in C. And the activity of the gene product thereof is the activity of dihydrolipoamide dehydrogenase.

Accordingly, in one embodiment, the process of the present invention for producing raffinose in a non-human organism, like a microorganism or a plant or a part thereof, comprises increasing or generating the activity of a gene product with the activity of a gene product conferring the activity of “dihydrolipoamide dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said Slr1096, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr1096, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr1096, or a functional equivalent or a homolog thereof as depicted in column 8 of Table II, application no. 17, preferably a homolog or functional equivalent as depicted in column 8 of Table II B, application no. 17, and being depicted in the same respective line as said Slr1096, and preferably the activity is increased non-targeted, whereby the respective line disclose in column 7 the fine chemical raffinose.

Accordingly, in one embodiment, the molecule which activity is to be increased in the process of the invention is the gene product with an activity as a “dihydrolipoamide dehydrogenase”, preferably it is the molecule of section (a) or (b) of this paragraph.

In particular, it was observed that in plants, especially in Arabidopsis thaliana, increasing or generating the activity of a gene product non-targeted with the activity of a “dihydrolipoamide dehydrogenase”, preferably being encoded by a gene comprising the nucleic acid sequence SEQ ID NO. 57734, preferably the coding region thereof, conferred the production of or the increase in raffinose compared with the wild type control.

The nucleic acid sequence of Slr1107 from Synechocystis sp., e.g. as shown in the respective line in column 5 of Table I, application no. 17, has been published in C. And the activity of the gene product thereof is the activity of slr1107-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “slr1107-protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said Slr1107, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr1107, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr1107, or a functional equivalent or a homolog thereof as depicted in column 8 of Table II, application no. 17, preferably a homolog or functional equivalent as depicted in column 8 of Table II B, application no. 17, and being depicted in the same respective line as said Slr1107, and preferably the activity is increased plastidic, whereby the respective line disclose in column 7 the fine chemical myo-inositol.

Accordingly, in one embodiment, the molecule which activity is to be increased in the process of the invention is the gene product with an activity as a “slr1107-protein”, preferably it is the molecule of section (a) or (b) of this paragraph.

In particular, it was observed that in plants, especially in Arabidopsis thaliana, increasing or generating the activity of a gene product plastidic with the activity of a “slr1107-protein”, preferably being encoded by a gene comprising the nucleic acid sequence SEQ ID NO. 101776, preferably the coding region thereof, conferred the production of or the increase in myo-inositol compared with the wild type control.

The nucleic acid sequence of Slr1350 from Synechocystis sp., e.g. as shown in the respective line in column 5 of Table I, application no. 17, has been published in C. And the activity of the gene product thereof is the activity of fatty acid desaturase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “fatty acid desaturase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Slr1350, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr1350, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr1350, or a functional equivalent or a homolog thereof as depicted in column 8 of Table II, application no. 17, preferably a homolog or functional equivalent as depicted in column 8 of Table II B, application no. 17, and being depicted in the same respective line as said Slr1350, and preferably the activity is increased mitochondrial, whereby the respective line disclose in column 7 the fine chemical glucose-1-phosphate.

Accordingly, in one embodiment, the molecule which activity is to be increased in the process of the invention is the gene product with an activity as a “fatty acid desaturase”, preferably it is the molecule of section (a) or (b) of this paragraph.

In particular, it was observed that in plants, especially in Arabidopsis thaliana, increasing or generating the activity of a gene product mitochondrial with the activity of a “fatty acid desaturase”, preferably being encoded by a gene comprising the nucleic acid sequence SEQ ID NO. 116713, preferably the coding region thereof, conferred the production of or the increase in glucose-1-phosphate compared with the wild type control.

The nucleic acid sequence of Slr1369 from Synechocystis sp., e.g. as shown in the respective line in column 5 of Table I, application no. 17, has been published in C. And the activity of the gene product thereof is the activity of CDP-diglyceride synthetase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “CDP-diglyceride synthetase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Slr1369, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr1369, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr1369, or a functional equivalent or a homolog thereof as depicted in column 8 of Table II, application no. 17, preferably a homolog or functional equivalent as depicted in column 8 of Table II B, application no. 17, and being depicted in the same respective line as said Slr1369, and preferably the activity is increased mitochondrial, whereby the respective line disclose in column 7 the fine chemical glucose-1-phosphate.

Accordingly, in one embodiment, the molecule which activity is to be increased in the process of the invention is the gene product with an activity as a “CDP-diglyceride synthetase”, preferably it is the molecule of section (a) or (b) of this paragraph.

In particular, it was observed that in plants, especially in Arabidopsis thaliana, increasing or generating the activity of a gene product mitochondrial with the activity of a “CDP-diglyceride synthetase”, preferably being encoded by a gene comprising the nucleic acid sequence SEQ ID NO. 58472, preferably the coding region thereof, conferred the production of or the increase in glucose-1-phosphate compared with the wild type control.

The nucleic acid sequence of Slr1369 from Synechocystis sp., e.g. as shown in the respective line in column 5 of Table I, application no. 17, has been published in C. And the activity of the gene product thereof is the activity of CDP-diglyceride synthetase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “CDP-diglyceride synthetase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said Slr1369, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr1369, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr1369, or a functional equivalent or a homolog thereof as depicted in column 8 of Table II, application no. 17, preferably a homolog or functional equivalent as depicted in column 8 of Table II B, application no. 17, and being depicted in the same respective line as said Slr1369, and preferably the activity is increased mitochondrial, whereby the respective line disclose in column 7 the fine chemical myo-inositol.

Accordingly, in one embodiment, the molecule which activity is to be increased in the process of the invention is the gene product with an activity as a “CDP-diglyceride synthetase”, preferably it is the molecule of section (a) or (b) of this paragraph.

In particular, it was observed that in plants, especially in Arabidopsis thaliana, increasing or generating the activity of a gene product mitochondrial with the activity of a “CDP-diglyceride synthetase”, preferably being encoded by a gene comprising the nucleic acid sequence SEQ ID NO. 58472, preferably the coding region thereof, conferred the production of or the increase in myo-inositol compared with the wild type control.

The nucleic acid sequence of Slr1492 from Synechocystis sp., e.g. as shown in the respective line in column 5 of Table I, application no. 17, has been published in C. And the activity of the gene product thereof is the activity of iron(III) dicitrate-binding protein.

Accordingly, in one embodiment, the process of the present invention for producing fructose in a non-human organism, like a microorganism or a plant or a part thereof, comprises increasing or generating the activity of a gene product with the activity of a gene product conferring the activity of “iron(III) dicitrate-binding protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said Slr1492, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr1492, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr1492, or a functional equivalent or a homolog thereof as depicted in column 8 of Table II, application no. 17, preferably a homolog or functional equivalent as depicted in column 8 of Table II B, application no. 17, and being depicted in the same respective line as said Slr1492, and preferably the activity is increased mitochondrial, whereby the respective line disclose in column 7 the fine chemical fructose.

Accordingly, in one embodiment, the molecule which activity is to be increased in the process of the invention is the gene product with an activity as a “iron(III) dicitrate-binding protein”, preferably it is the molecule of section (a) or (b) of this paragraph.

In particular, it was observed that in plants, especially in Arabidopsis thaliana, increasing or generating the activity of a gene product mitochondrial with the activity of a “iron(III) dicitratebinding protein”, preferably being encoded by a gene comprising the nucleic acid sequence SEQ ID NO. 58668, preferably the coding region thereof, conferred the production of or the increase in fructose compared with the wild type control.

The nucleic acid sequence of Slr1655 from Synechocystis sp., e.g. as shown in the respective line in column 5 of Table I, application no. 17, has been published in C. And the activity of the gene product thereof is the activity of photosystem I reaction center subunit XI.

Accordingly, in one embodiment, the process of the present invention for producing sucrose in a non-human organism, like a microorganism or a plant or a part thereof, comprises increasing or generating the activity of a gene product with the activity of a gene product conferring the activity of “photosystem I reaction center subunit XI”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said Slr1655, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr1655, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr1655, or a functional equivalent or a homolog thereof as depicted in column 8 of Table II, application no. 17, preferably a homolog or functional equivalent as depicted in column 8 of Table II B, application no. 17, and being depicted in the same respective line as said Slr1655, and preferably the activity is increased mitochondrial, whereby the respective line disclose in column 7 the fine chemical sucrose.

Accordingly, in one embodiment, the molecule which activity is to be increased in the process of the invention is the gene product with an activity as a “photosystem I reaction center subunit XI”, preferably it is the molecule of section (a) or (b) of this paragraph.

In particular, it was observed that in plants, especially in Arabidopsis thaliana, increasing or generating the activity of a gene product mitochondrial with the activity of a “photosystem I reaction center subunit XI”, preferably being encoded by a gene comprising the nucleic acid sequence SEQ ID NO. 12070, preferably the coding region thereof, conferred the production of or the increase in sucrose compared with the wild type control.

The nucleic acid sequence of Slr1739 from Synechocystis sp., e.g. as shown in the respective line in column 5 of Table I, application no. 17, has been published in C. And the activity of the gene product thereof is the activity of photosystem II protein.

Accordingly, in one embodiment, the process of the present invention for producing fructose in a non-human organism, like a microorganism or a plant or a part thereof, comprises increasing or generating the activity of a gene product with the activity of a gene product conferring the activity of “photosystem II protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said Slr1739, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr1739, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr1739, or a functional equivalent or a homolog thereof as depicted in column 8 of Table II, application no. 17, preferably a homolog or functional equivalent as depicted in column 8 of Table II B, application no. 17, and being depicted in the same respective line as said Slr1739, and preferably the activity is increased plastidic, whereby the respective line disclose in column 7 the fine chemical fructose.

Accordingly, in one embodiment, the molecule which activity is to be increased in the process of the invention is the gene product with an activity as a “photosystem II protein”, preferably it is the molecule of section (a) or (b) of this paragraph.

In particular, it was observed that in plants, especially in Arabidopsis thaliana, increasing or generating the activity of a gene product plastidic with the activity of a “photosystem II protein”, preferably being encoded by a gene comprising the nucleic acid sequence SEQ ID NO. 58731, preferably the coding region thereof, conferred the production of or the increase in fructose compared with the wild type control.

The nucleic acid sequence of Slr1742 from Synechocystis sp., e.g. as shown in the respective line in column 5 of Table I, application no. 17, has been published in C. And the activity of the gene product thereof is the activity of glutamine amidotransferase.

Accordingly, in one embodiment, the process of the present invention for producing fructose in a non-human organism, like a microorganism or a plant or a part thereof, comprises increasing or generating the activity of a gene product with the activity of a gene product conferring the activity of “glutamine amidotransferase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said Slr1742, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr1742, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr1742, or a functional equivalent or a homolog thereof as depicted in column 8 of Table II, application no. 17, preferably a homolog or functional equivalent as depicted in column 8 of Table II B, application no. 17, and being depicted in the same respective line as said Slr1742, and preferably the activity is increased plastidic, whereby the respective line disclose in column 7 the fine chemical fructose.

Accordingly, in one embodiment, the molecule which activity is to be increased in the process of the invention is the gene product with an activity as a “glutamine amidotransferase”, preferably it is the molecule of section (a) or (b) of this paragraph.

In particular, it was observed that in plants, especially in Arabidopsis thaliana, increasing or generating the activity of a gene product plastidic with the activity of a “glutamine amidotransferase”, preferably being encoded by a gene comprising the nucleic acid sequence SEQ ID NO. 58751, preferably the coding region thereof, conferred the production of or the increase in fructose compared with the wild type control.

The nucleic acid sequence of Slr1742 from Synechocystis sp., e.g. as shown in the respective line in column 5 of Table I, application no. 17, has been published in C. And the activity of the gene product thereof is the activity of glutamine amidotransferase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “glutamine amidotransferase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said Slr1742, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr1742, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr1742, or a functional equivalent or a homolog thereof as depicted in column 8 of Table II, application no. 17, preferably a homolog or functional equivalent as depicted in column 8 of Table II B, application no. 17, and being depicted in the same respective line as said Slr1742, and preferably the activity is increased plastidic, whereby the respective line disclose in column 7 the fine chemical myo-inositol.

Accordingly, in one embodiment, the molecule which activity is to be increased in the process of the invention is the gene product with an activity as a “glutamine amidotransferase”, preferably it is the molecule of section (a) or (b) of this paragraph.

In particular, it was observed that in plants, especially in Arabidopsis thaliana, increasing or generating the activity of a gene product plastidic with the activity of a “glutamine amidotransferase”, preferably being encoded by a gene comprising the nucleic acid sequence SEQ ID NO. 58751, preferably the coding region thereof, conferred the production of or the increase in myo-inositol compared with the wild type control.

The nucleic acid sequence of Slr1743 from Synechocystis sp., e.g. as shown in the respective line in column 5 of Table I, application no. 17, has been published in C. And the activity of the gene product thereof is the activity of NADH dehydrogenase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “NADH dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Slr1743, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr1743, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr1743, or a functional equivalent or a homolog thereof as depicted in column 8 of Table II, application no. 17, preferably a homolog or functional equivalent as depicted in column 8 of Table II B, application no. 17, and being depicted in the same respective line as said Slr1743, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “NADH dehydrogenase”, preferably it is the molecule of section (a) or (b) of this paragraph.

In particular, it was observed that in plants, especially in Arabidopsis thaliana, increasing or generating the activity of a gene product plastidic with the activity of a “NADH dehydrogenase”, preferably being encoded by a gene comprising the nucleic acid sequence SEQ ID NO. 147457, preferably the coding region thereof, conferred the production of or the increase in glucose-1-phosphate compared with the wild type control.

The nucleic acid sequence of Slr1779 from Synechocystis sp., e.g. as shown in the respective line in column 5 of Table I, application no. 17, has been published in C. And the activity of the gene product thereof is the activity of pyridoxal phosphate biosynthetic protein.

Accordingly, in one embodiment, the process of the present invention for producing raffinose in a non-human organism, like a microorganism or a plant or a part thereof, comprises increasing or generating the activity of a gene product with the activity of a gene product conferring the activity of “pyridoxal phosphate biosynthetic protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said Slr1779, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr1779, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr1779, or a functional equivalent or a homolog thereof as depicted in column 8 of Table II, application no. 17, preferably a homolog or functional equivalent as depicted in column 8 of Table II B, application no. 17, and being depicted in the same respective line as said Slr1779, and preferably the activity is increased plastidic, whereby the respective line disclose in column 7 the fine chemical raffinose.

Accordingly, in one embodiment, the molecule which activity is to be increased in the process of the invention is the gene product with an activity as a “pyridoxal phosphate biosynthetic protein”, preferably it is the molecule of section (a) or (b) of this paragraph.

In particular, it was observed that in plants, especially in Arabidopsis thaliana, increasing or generating the activity of a gene product plastidic with the activity of a “pyridoxal phosphate biosynthetic protein”, preferably being encoded by a gene comprising the nucleic acid sequence SEQ ID NO. 147637, preferably the coding region thereof, conferred the production of or the increase in raffinose compared with the wild type control.

The nucleic acid sequence of Slr1791 from Synechocystis sp., e.g. as shown in the respective line in column 5 of Table I, application no. 17, has been published in C. And the activity of the gene product thereof is the activity of phosphoadenosine phosphosulfate reductase .

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “phosphoadenosine phosphosulfate reductase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Slr1791, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr1791, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr1791, or a functional equivalent or a homolog thereof as depicted in column 8 of Table II, application no. 17, preferably a homolog or functional equivalent as depicted in column 8 of Table II B, application no. 17, and being depicted in the same respective line as said Slr1791, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “phosphoadenosine phosphosulfate reductase”, preferably it is the molecule of section (a) or (b) of this paragraph.

In particular, it was observed that in plants, especially in Arabidopsis thaliana, increasing or generating the activity of a gene product plastidic with the activity of a “phosphoadenosine phosphosulfate reductase”, preferably being encoded by a gene comprising the nucleic acid sequence SEQ ID NO. 12140, preferably the coding region thereof, conferred the production of or the increase in glucose-1-phosphate compared with the wild type control.

The nucleic acid sequence of Slr2072 from Synechocystis sp., e.g. as shown in the respective line in column 5 of Table I, application no. 17, has been published in C. And the activity of the gene product thereof is the activity of threonine dehydratase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “threonine dehydratase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Slr2072, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr2072, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr2072, or a functional equivalent or a homolog thereof as depicted in column 8 of Table II, application no. 17, preferably a homolog or functional equivalent as depicted in column 8 of Table II B, application no. 17, and being depicted in the same respective line as said Slr2072, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “threonine dehydratase”, preferably it is the molecule of section (a) or (b) of this paragraph.

In particular, it was observed that in plants, especially in Arabidopsis thaliana, increasing or generating the activity of a gene product plastidic with the activity of a “threonine dehydratase”, preferably being encoded by a gene comprising the nucleic acid sequence SEQ ID NO. 12341, preferably the coding region thereof, conferred the production of or the increase in glucose-1-phosphate compared with the wild type control.

The nucleic acid sequence of Slr2072 from Synechocystis sp., e.g. as shown in the respective line in column 5 of Table I, application no. 17, has been published in C. And the activity of the gene product thereof is the activity of threonine dehydratase.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “threonine dehydratase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said Slr2072, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr2072, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr2072, or a functional equivalent or a homolog thereof as depicted in column 8 of Table II, application no. 17, preferably a homolog or functional equivalent as depicted in column 8 of Table II B, application no. 17, and being depicted in the same respective line as said Slr2072, and preferably the activity is increased plastidic, whereby the respective line disclose in column 7 the fine chemical 3,4,5-trihydroxypentanoic acid.

Accordingly, in one embodiment, the molecule which activity is to be increased in the process of the invention is the gene product with an activity as a “threonine dehydratase”, preferably it is the molecule of section (a) or (b) of this paragraph.

In particular, it was observed that in plants, especially in Arabidopsis thaliana, increasing or generating the activity of a gene product plastidic with the activity of a “threonine dehydratase”, preferably being encoded by a gene comprising the nucleic acid sequence SEQ ID NO. 12341, preferably the coding region thereof, conferred the production of or the increase in 3,4,5-trihydroxypentanoic acid compared with the wild type control.

The nucleic acid sequence of Slr2124 from Synechocystis sp., e.g. as shown in the respective line in column 5 of Table I, application no. 17, has been published in C. And the activity of the gene product thereof is the activity of short-chain alcohol dehydrogenase family.

Accordingly, in one embodiment, the process of the present invention for producing fructose in a non-human organism, like a microorganism or a plant or a part thereof, comprises increasing or generating the activity of a gene product with the activity of a gene product conferring the activity of “short-chain alcohol dehydrogenase family”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said Slr2124, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr2124, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr2124, or a functional equivalent or a homolog thereof as depicted in column 8 of Table II, application no. 17, preferably a homolog or functional equivalent as depicted in column 8 of Table II B, application no. 17, and being depicted in the same respective line as said Slr2124, and preferably the activity is increased plastidic, whereby the respective line disclose in column 7 the fine chemical fructose.

Accordingly, in one embodiment, the molecule which activity is to be increased in the process of the invention is the gene product with an activity as a “short-chain alcohol dehydrogenase family”, preferably it is the molecule of section (a) or (b) of this paragraph.

In particular, it was observed that in plants, especially in Arabidopsis thaliana, increasing or generating the activity of a gene product plastidic with the activity of a “short-chain alcohol dehydrogenase family”, preferably being encoded by a gene comprising the nucleic acid sequence SEQ ID NO. 59370, preferably the coding region thereof, conferred the production of or the increase in fructose compared with the wild type control.

The nucleic acid sequence of Slr2124 from Synechocystis sp., e.g. as shown in the respective line in column 5 of Table I, application no. 17, has been published in C. And the activity of the gene product thereof is the activity of short-chain alcohol dehydrogenase family.

Accordingly, in one embodiment, the process of the present invention for producing glucose in a non-human organism, like a microorganism or a plant or a part thereof, comprises increasing or generating the activity of a gene product with the activity of a gene product conferring the activity of “short-chain alcohol dehydrogenase family”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said Slr2124, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr2124, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr2124, or a functional equivalent or a homolog thereof as depicted in column 8 of Table II, application no. 17, preferably a homolog or functional equivalent as depicted in column 8 of Table II B, application no. 17, and being depicted in the same respective line as said Slr2124, and preferably the activity is increased plastidic, whereby the respective line disclose in column 7 the fine chemical glucose.

Accordingly, in one embodiment, the molecule which activity is to be increased in the process of the invention is the gene product with an activity as a “short-chain alcohol dehydrogenase family”, preferably it is the molecule of section (a) or (b) of this paragraph.

In particular, it was observed that in plants, especially in Arabidopsis thaliana, increasing or generating the activity of a gene product plastidic with the activity of a “short-chain alcohol dehydrogenase family”, preferably being encoded by a gene comprising the nucleic acid sequence SEQ ID NO. 59370, preferably the coding region thereof, conferred the production of or the increase in glucose compared with the wild type control.

The nucleic acid sequence of Slr2124 from Synechocystis sp., e.g. as shown in the respective line in column 5 of Table I, application no. 17, has been published in C. And the activity of the gene product thereof is the activity of short-chain alcohol dehydrogenase family.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “short-chain alcohol dehydrogenase family”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Slr2124, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr2124, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr2124, or a functional equivalent or a homolog thereof as depicted in column 8 of Table II, application no. 17, preferably a homolog or functional equivalent as depicted in column 8 of Table II B, application no. 17, and being depicted in the same respective line as said Slr2124, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “short-chain alcohol dehydrogenase family”, preferably it is the molecule of section (a) or (b) of this paragraph.

In particular, it was observed that in plants, especially in Arabidopsis thaliana, increasing or generating the activity of a gene product plastidic with the activity of a “short-chain alcohol dehydrogenase family”, preferably being encoded by a gene comprising the nucleic acid sequence SEQ ID NO. 59370, preferably the coding region thereof, conferred the production of or the increase in glucose-1-phosphate compared with the wild type control.

The nucleic acid sequence of Slr2124 from Synechocystis sp., e.g. as shown in the respective line in column 5 of Table I, application no. 17, has been published in C. And the activity of the gene product thereof is the activity of short-chain alcohol dehydrogenase family.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “short-chain alcohol dehydrogenase family”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said Slr2124, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr2124, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr2124, or a functional equivalent or a homolog thereof as depicted in column 8 of Table II, application no. 17, preferably a homolog or functional equivalent as depicted in column 8 of Table II B, application no. 17, and being depicted in the same respective line as said Slr2124, and preferably the activity is increased plastidic, whereby the respective line disclose in column 7 the fine chemical myo-inositol.

Accordingly, in one embodiment, the molecule which activity is to be increased in the process of the invention is the gene product with an activity as a “short-chain alcohol dehydrogenase family”, preferably it is the molecule of section (a) or (b) of this paragraph.

In particular, it was observed that in plants, especially in Arabidopsis thaliana, increasing or generating the activity of a gene product plastidic with the activity of a “short-chain alcohol dehydrogenase family”, preferably being encoded by a gene comprising the nucleic acid sequence SEQ ID NO. 59370, preferably the coding region thereof, conferred the production of or the increase in myo-inositol compared with the wild type control.

The nucleic acid sequence of Slr2124 from Synechocystis sp., e.g. as shown in the respective line in column 5 of Table I, application no. 17, has been published in C. And the activity of the gene product thereof is the activity of short-chain alcohol dehydrogenase family.

Accordingly, in one embodiment, the process of the present invention for producing sucrose in a non-human organism, like a microorganism or a plant or a part thereof, comprises increasing or generating the activity of a gene product with the activity of a gene product conferring the activity of “short-chain alcohol dehydrogenase family”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said Slr2124, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr2124, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr2124, or a functional equivalent or a homolog thereof as depicted in column 8 of Table II, application no. 17, preferably a homolog or functional equivalent as depicted in column 8 of Table II B, application no. 17, and being depicted in the same respective line as said Slr2124, and preferably the activity is increased plastidic, whereby the respective line disclose in column 7 the fine chemical sucrose.

Accordingly, in one embodiment, the molecule which activity is to be increased in the process of the invention is the gene product with an activity as a “short-chain alcohol dehydrogenase family”, preferably it is the molecule of section (a) or (b) of this paragraph.

In particular, it was observed that in plants, especially in Arabidopsis thaliana, increasing or generating the activity of a gene product plastidic with the activity of a “short-chain alcohol dehydrogenase family”, preferably being encoded by a gene comprising the nucleic acid sequence SEQ ID NO. 59370, preferably the coding region thereof, conferred the production of or the increase in sucrose compared with the wild type control.

The nucleic acid sequence of Slr2132 from Synechocystis sp., e.g. as shown in the respective line in column 5 of Table I, application no. 17, has been published in C. And the activity of the gene product thereof is the activity of phosphate acetyltransferase.

Accordingly, in one embodiment, the process of the present invention for producing raffinose in a non-human organism, like a microorganism or a plant or a part thereof, comprises increasing or generating the activity of a gene product with the activity of a gene product conferring the activity of “phosphate acetyltransferase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said Slr2132, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Slr2132, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Slr2132, or a functional equivalent or a homolog thereof as depicted in column 8 of Table II, application no. 17, preferably a homolog or functional equivalent as depicted in column 8 of Table II B, application no. 17, and being depicted in the same respective line as said Slr2132, and preferably the activity is increased mitochondrial, whereby the respective line disclose in column 7 the fine chemical raffinose.

Accordingly, in one embodiment, the molecule which activity is to be increased in the process of the invention is the gene product with an activity as a “phosphate acetyltransferase”, preferably it is the molecule of section (a) or (b) of this paragraph.

In particular, it was observed that in plants, especially in Arabidopsis thaliana, increasing or generating the activity of a gene product mitochondrial with the activity of a “phosphate acetyltransferase”, preferably being encoded by a gene comprising the nucleic acid sequence SEQ ID NO. 147836, preferably the coding region thereof, conferred the production of or the increase in raffinose compared with the wild type control.

The nucleic acid sequence of Smr0004 from Synechocystis sp., e.g. as shown in the respective line in column 5 of Table I, application no. 17, has been published in C. And the activity of the gene product thereof is the activity of photosystem I reaction centre subunit.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “photosystem I reaction centre subunit”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Smr0004, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Smr0004, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Smr0004, or a functional equivalent or a homolog thereof as depicted in column 8 of Table II, application no. 17, preferably a homolog or functional equivalent as depicted in column 8 of Table II B, application no. 17, and being depicted in the same respective line as said Smr0004, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “photosystem I reaction centre subunit”, preferably it is the molecule of section (a) or (b) of this paragraph.

In particular, it was observed that in plants, especially in Arabidopsis thaliana, increasing or generating the activity of a gene product plastidic with the activity of a “photosystem I reaction centre subunit”, preferably being encoded by a gene comprising the nucleic acid sequence SEQ ID NO. 136907, preferably the coding region thereof, conferred the production of or the increase in glucose-1-phosphate compared with the wild type control.

The nucleic acid sequence of Smr0004 from Synechocystis sp., e.g. as shown in the respective line in column 5 of Table I, application no. 17, has been published in C. And the activity of the gene product thereof is the activity of photosystem I reaction centre subunit.

Accordingly, in one embodiment, the process of the present invention for producing raffinose in a non-human organism, like a microorganism or a plant or a part thereof, comprises increasing or generating the activity of a gene product with the activity of a gene product conferring the activity of “photosystem I reaction centre subunit”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said Smr0004, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Smr0004, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Smr0004, or a functional equivalent or a homolog thereof as depicted in column 8 of Table II, application no. 17, preferably a homolog or functional equivalent as depicted in column 8 of Table II B, application no. 17, and being depicted in the same respective line as said Smr0004, and preferably the activity is increased plastidic, whereby the respective line disclose in column 7 the fine chemical raffinose.

Accordingly, in one embodiment, the molecule which activity is to be increased in the process of the invention is the gene product with an activity as a “photosystem I reaction centre subunit”, preferably it is the molecule of section (a) or (b) of this paragraph.

In particular, it was observed that in plants, especially in Arabidopsis thaliana, increasing or generating the activity of a gene product plastidic with the activity of a “photosystem I reaction centre subunit”, preferably being encoded by a gene comprising the nucleic acid sequence SEQ ID NO. 136907, preferably the coding region thereof, conferred the production of or the increase in raffinose compared with the wild type control.

The nucleic acid sequence of Smr0004 from Synechocystis sp., e.g. as shown in the respective line in column 5 of Table I, application no. 17, has been published in C. And the activity of the gene product thereof is the activity of photosystem I reaction centre subunit.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “photosystem I reaction centre subunit”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said Smr0004, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Smr0004, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Smr0004, or a functional equivalent or a homolog thereof as depicted in column 8 of Table II, application no. 17, preferably a homolog or functional equivalent as depicted in column 8 of Table II B, application no. 17, and being depicted in the same respective line as said Smr0004, and preferably the activity is increased plastidic, whereby the respective line disclose in column 7 the fine chemical 3,4,5-trihydroxypentanoic acid.

Accordingly, in one embodiment, the molecule which activity is to be increased in the process of the invention is the gene product with an activity as a “photosystem I reaction centre subunit”, preferably it is the molecule of section (a) or (b) of this paragraph.

In particular, it was observed that in plants, especially in Arabidopsis thaliana, increasing or generating the activity of a gene product plastidic with the activity of a “photosystem I reaction centre subunit”, preferably being encoded by a gene comprising the nucleic acid sequence SEQ ID NO. 136907, preferably the coding region thereof, conferred the production of or the increase in 3,4,5-trihydroxypentanoic acid compared with the wild type control.

The nucleic acid sequence of TTC0019 from Thermus thermophilus, e.g. as shown in the respective line in column 5 of Table I, application no. 17, has been published in E. And the activity of the gene product thereof is the activity of Sec-independent protein translocase subunit.

Accordingly, in one embodiment, the process of the present invention for producing fructose in a non-human organism, like a microorganism or a plant or a part thereof, comprises increasing or generating the activity of a gene product with the activity of a gene product conferring the activity of “Sec-independent protein translocase subunit”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said TTC0019, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said TTC0019, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said TTC0019, or a functional equivalent or a homolog thereof as depicted in column 8 of Table II, application no. 17, preferably a homolog or functional equivalent as depicted in column 8 of Table II B, application no. 17, and being depicted in the same respective line as said TTC0019, and preferably the activity is increased non-targeted, whereby the respective line disclose in column 7 the fine chemical fructose.

Accordingly, in one embodiment, the molecule which activity is to be increased in the process of the invention is the gene product with an activity as a “Sec-independent protein translocase subunit”, preferably it is the molecule of section (a) or (b) of this paragraph.

In particular, it was observed that in plants, especially in Arabidopsis thaliana, increasing or generating the activity of a gene product non-targeted with the activity of a “Sec-independent protein translocase subunit”, preferably being encoded by a gene comprising the nucleic acid sequence SEQ ID NO. 12698, preferably the coding region thereof, conferred the production of or the increase in fructose compared with the wild type control.

The nucleic acid sequence of TTC0019 from Thermus thermophilus, e.g. as shown in the respective line in column 5 of Table I, application no. 17, has been published in E. And the activity of the gene product thereof is the activity of Sec-independent protein translocase subunit.

Accordingly, in one embodiment, the process of the present invention for producing glucose in a non-human organism, like a microorganism or a plant or a part thereof, comprises increasing or generating the activity of a gene product with the activity of a gene product conferring the activity of “Sec-independent protein translocase subunit”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said TTC0019, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said TTC0019, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said TTC0019, or a functional equivalent or a homolog thereof as depicted in column 8 of Table II, application no. 17, preferably a homolog or functional equivalent as depicted in column 8 of Table II B, application no. 17, and being depicted in the same respective line as said TTC0019, and preferably the activity is increased non-targeted, whereby the respective line disclose in column 7 the fine chemical glucose.

Accordingly, in one embodiment, the molecule which activity is to be increased in the process of the invention is the gene product with an activity as a “Sec-independent protein translocase subunit”, preferably it is the molecule of section (a) or (b) of this paragraph.

In particular, it was observed that in plants, especially in Arabidopsis thaliana, increasing or generating the activity of a gene product non-targeted with the activity of a “Sec-independent protein translocase subunit”, preferably being encoded by a gene comprising the nucleic acid sequence SEQ ID NO. 12698, preferably the coding region thereof, conferred the production of or the increase in glucose compared with the wild type control.

The nucleic acid sequence of TTC0019 from Thermus thermophilus, e.g. as shown in the respective line in column 5 of Table I, application no. 17, has been published in E. And the activity of the gene product thereof is the activity of Sec-independent protein translocase subunit.

Accordingly, in one embodiment, the process of the present invention for producing sedoheptulose-7-phosphate in a non-human organism, like a microorganism or a plant or a part thereof, comprises increasing or generating the activity of a gene product with the activity of a gene product conferring the activity of “Sec-independent protein translocase subunit”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical sedoheptulose-7-phosphate), application no. 17, preferably the coding region thereof, or a homolog or a fragment thereof, and being depicted in the same respective line as said TTC0019, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said TTC0019, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said TTC0019, or a functional equivalent or a homolog thereof as depicted in column 8 of Table II, application no. 17, preferably a homolog or functional equivalent as depicted in column 8 of Table II B, application no. 17, and being depicted in the same respective line as said TTC0019, and preferably the activity is increased non-targeted, whereby the respective line disclose in column 7 the fine chemical sedoheptulose-7-phosphate.

Accordingly, in one embodiment, the molecule which activity is to be increased in the process of the invention is the gene product with an activity as a “Sec-independent protein translocase subunit”, preferably it is the molecule of section (a) or (b) of this paragraph.

In particular, it was observed that in plants, especially in Arabidopsis thaliana, increasing or generating the activity of a gene product non-targeted with the activity of a “Sec-independent protein translocase subunit”, preferably being encoded by a gene comprising the nucleic acid sequence SEQ ID NO. 12698, preferably the coding region thereof, conferred the production of or the increase in sedoheptulose-7-phosphate compared with the wild type control.

The nucleic acid sequence of TTC0035 from Thermus thermophilus, e.g. as shown in the respective line in column 5 of Table I, application no. 17, has been published in E. And the activity of the gene product thereof is the activity of cell division protein.

Accordingly, in one embodiment, the process of the present invention for producing fructose in a non-human organism, like a microorganism or a plant or a part thereof, comprises increasing or generating the activity of a gene product with the activity of a gene product conferring the activity of “cell division protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said TTC0035, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said TTC0035, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said TTC0035, or a functional equivalent or a homolog thereof as depicted in column 8 of Table II, application no. 17, preferably a homolog or functional equivalent as depicted in column 8 of Table II B, application no. 17, and being depicted in the same respective line as said TTC0035, and preferably the activity is increased non-targeted, whereby the respective line disclose in column 7 the fine chemical fructose.

Accordingly, in one embodiment, the molecule which activity is to be increased in the process of the invention is the gene product with an activity as a “cell division protein”, preferably it is the molecule of section (a) or (b) of this paragraph.

In particular, it was observed that in plants, especially in Arabidopsis thaliana, increasing or generating the activity of a gene product non-targeted with the activity of a “cell division protein”, preferably being encoded by a gene comprising the nucleic acid sequence SEQ ID NO. 60301, preferably the coding region thereof, conferred the production of or the increase in fructose compared with the wild type control.

The nucleic acid sequence of TTC0035 from Thermus thermophilus, e.g. as shown in the respective line in column 5 of Table I, application no. 17, has been published in E. And the activity of the gene product thereof is the activity of cell division protein.

Accordingly, in one embodiment, the process of the present invention for producing glucose in a non-human organism, like a microorganism or a plant or a part thereof, comprises increasing or generating the activity of a gene product with the activity of a gene product conferring the activity of “cell division protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said TTC0035, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said TTC0035, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said TTC0035, or a functional equivalent or a homolog thereof as depicted in column 8 of Table II, application no. 17, preferably a homolog or functional equivalent as depicted in column 8 of Table II B, application no. 17, and being depicted in the same respective line as said TTC0035, and preferably the activity is increased non-targeted, whereby the respective line disclose in column 7 the fine chemical glucose.

Accordingly, in one embodiment, the molecule which activity is to be increased in the process of the invention is the gene product with an activity as a “cell division protein”, preferably it is the molecule of section (a) or (b) of this paragraph.

In particular, it was observed that in plants, especially in Arabidopsis thaliana, increasing or generating the activity of a gene product non-targeted with the activity of a “cell division protein”, preferably being encoded by a gene comprising the nucleic acid sequence SEQ ID NO. 60301, preferably the coding region thereof, conferred the production of or the increase in glucose compared with the wild type control.

The nucleic acid sequence of TTC0035 from Thermus thermophilus, e.g. as shown in the respective line in column 5 of Table I, application no. 17, has been published in E. And the activity of the gene product thereof is the activity of cell division protein.

Accordingly, in one embodiment, the process of the present invention for producing sucrose in a non-human organism, like a microorganism or a plant or a part thereof, comprises increasing or generating the activity of a gene product with the activity of a gene product conferring the activity of “cell division protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said TTC0035, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said TTC0035, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said TTC0035, or a functional equivalent or a homolog thereof as depicted in column 8 of Table II, application no. 17, preferably a homolog or functional equivalent as depicted in column 8 of Table II B, application no. 17, and being depicted in the same respective line as said TTC0035, and preferably the activity is increased non-targeted, whereby the respective line disclose in column 7 the fine chemical sucrose.

Accordingly, in one embodiment, the molecule which activity is to be increased in the process of the invention is the gene product with an activity as a “cell division protein”, preferably it is the molecule of section (a) or (b) of this paragraph.

In particular, it was observed that in plants, especially in Arabidopsis thaliana, increasing or generating the activity of a gene product non-targeted with the activity of a “cell division protein”, preferably being encoded by a gene comprising the nucleic acid sequence SEQ ID NO. 60301, preferably the coding region thereof, conferred the production of or the increase in sucrose compared with the wild type control.

The nucleic acid sequence of TTC0216 from Thermus thermophilus, e.g. as shown in the respective line in column 5 of Table I, application no. 17, has been published in E. And the activity of the gene product thereof is the activity of branched-chain amino acid ABC transporter permease protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “branched-chain amino acid ABC transporter permease protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said TTC0216, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said TTC0216, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said TTC0216, or a functional equivalent or a homolog thereof as depicted in column 8 of Table II, application no. 17, preferably a homolog or functional equivalent as depicted in column 8 of Table II B, application no. 17, and being depicted in the same respective line as said TTC0216, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “branched-chain amino acid ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “branched-chain amino acid ABC transporter permease protein”, preferably being encoded by a gene comprising the nucleic acid sequence SEQ ID NO. 60859, preferably the coding region thereof, conferred the production of or the increase in glucose-1-phosphate compared with the wild type control.

The nucleic acid sequence of TTC0216 from Thermus thermophilus, e.g. as shown in the respective line in column 5 of Table I, application no. 17, has been published in E. And the activity of the gene product thereof is the activity of branched-chain amino acid ABC transporter permease protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “branched-chain amino acid ABC transporter permease protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said TTC0216, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said TTC0216, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said TTC0216, or a functional equivalent or a homolog thereof as depicted in column 8 of Table II, application no. 17, preferably a homolog or functional equivalent as depicted in column 8 of Table II B, application no. 17, and being depicted in the same respective line as said TTC0216, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “branched-chain amino acid ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “branched-chain amino acid ABC transporter permease protein”, preferably being encoded by a gene comprising the nucleic acid sequence SEQ ID NO. 60859, preferably the coding region thereof, conferred the production of or the increase in 3,4,5-trihydroxypentanoic acid compared with the wild type control.

The nucleic acid sequence of TTC0865 from Thermus thermophilus, e.g. as shown in the respective line in column 5 of Table I, application no. 17, has been published in E. And the activity of the gene product thereof is the activity of 3-isopropylmalate dehydratase large subunit.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “3-isopropylmalate dehydratase large subunit”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said TTC0865, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said TTC0865, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said TTC0865, or a functional equivalent or a homolog thereof as depicted in column 8 of Table II, application no. 17, preferably a homolog or functional equivalent as depicted in column 8 of Table II B, application no. 17, and being depicted in the same respective line as said TTC0865, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “3-isopropylmalate dehydratase large subunit”, preferably it is the molecule of section (a) or (b) of this paragraph.

In particular, it was observed that in plants, especially in Arabidopsis thaliana, increasing or generating the activity of a gene product non-targeted with the activity of a “3-isopropylmalate dehydratase large subunit”, preferably being encoded by a gene comprising the nucleic acid sequence SEQ ID NO. 147944, preferably the coding region thereof, conferred the production of or the increase in myo-inositol compared with the wild type control.

The nucleic acid sequence of TTC1193 from Thermus thermophilus, e.g. as shown in the respective line in column 5 of Table I, application no. 17, has been published in E. And the activity of the gene product thereof is the activity of multiple antibiotic resistance protein.

Accordingly, in one embodiment, the process of the present invention for producing fructose in a non-human organism, like a microorganism or a plant or a part thereof, comprises increasing or generating the activity of a gene product with the activity of a gene product conferring the activity of “multiple antibiotic resistance protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said TTC1193, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said TTC1193, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said TTC1193, or a functional equivalent or a homolog thereof as depicted in column 8 of Table II, application no. 17, preferably a homolog or functional equivalent as depicted in column 8 of Table II B, application no. 17, and being depicted in the same respective line as said TTC1193, and preferably the activity is increased non-targeted, whereby the respective line disclose in column 7 the fine chemical fructose.

Accordingly, in one embodiment, the molecule which activity is to be increased in the process of the invention is the gene product with an activity as a “multiple antibiotic resistance protein”, preferably it is the molecule of section (a) or (b) of this paragraph.

In particular, it was observed that in plants, especially in Arabidopsis thaliana, increasing or generating the activity of a gene product non-targeted with the activity of a “multiple antibiotic resistance protein”, preferably being encoded by a gene comprising the nucleic acid sequence SEQ ID NO. 61723, preferably the coding region thereof, conferred the production of or the increase in fructose compared with the wild type control.

The nucleic acid sequence of TTC1193 from Thermus thermophilus, e.g. as shown in the respective line in column 5 of Table I, application no. 17, has been published in E. And the activity of the gene product thereof is the activity of multiple antibiotic resistance protein.

Accordingly, in one embodiment, the process of the present invention for producing glucose in a non-human organism, like a microorganism or a plant or a part thereof, comprises increasing or generating the activity of a gene product with the activity of a gene product conferring the activity of “multiple antibiotic resistance protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said TTC1193, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said TTC1193, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said TTC1193, or a functional equivalent or a homolog thereof as depicted in column 8 of Table II, application no. 17, preferably a homolog or functional equivalent as depicted in column 8 of Table II B, application no. 17, and being depicted in the same respective line as said TTC1193, and preferably the activity is increased non-targeted, whereby the respective line disclose in column 7 the fine chemical glucose.

Accordingly, in one embodiment, the molecule which activity is to be increased in the process of the invention is the gene product with an activity as a “multiple antibiotic resistance protein”, preferably it is the molecule of section (a) or (b) of this paragraph.

In particular, it was observed that in plants, especially in Arabidopsis thaliana, increasing or generating the activity of a gene product non-targeted with the activity of a “multiple antibiotic resistance protein”, preferably being encoded by a gene comprising the nucleic acid sequence SEQ ID NO. 61723, preferably the coding region thereof, conferred the production of or the increase in glucose compared with the wild type control.

The nucleic acid sequence of TTC1193 from Thermus thermophilus, e.g. as shown in the respective line in column 5 of Table I, application no. 17, has been published in E. And the activity of the gene product thereof is the activity of multiple antibiotic resistance protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “multiple antibiotic resistance protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said TTC1193, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said TTC1193, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said TTC1193, or a functional equivalent or a homolog thereof as depicted in column 8 of Table II, application no. 17, preferably a homolog or functional equivalent as depicted in column 8 of Table II B, application no. 17, and being depicted in the same respective line as said TTC1193, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “multiple antibiotic resistance protein”, preferably it is the molecule of section (a) or (b) of this paragraph.

In particular, it was observed that in plants, especially in Arabidopsis thaliana, increasing or generating the activity of a gene product non-targeted with the activity of a “multiple antibiotic resistance protein”, preferably being encoded by a gene comprising the nucleic acid sequence SEQ ID NO. 61723, preferably the coding region thereof, conferred the production of or the increase in glucose-1-phosphate compared with the wild type control.

The nucleic acid sequence of TTC1386 from Thermus thermophilus, e.g. as shown in the respective line in column 5 of Table I, application no. 17, has been published in E. And the activity of the gene product thereof is the activity of TTC1386-protein.

Accordingly, in one embodiment, the process of the present invention for producing fructose in a non-human organism, like a microorganism or a plant or a part thereof, comprises increasing or generating the activity of a gene product with the activity of a gene product conferring the activity of “TTC1386-protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said TTC1386, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said TTC1386, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said TTC1386, or a functional equivalent or a homolog thereof as depicted in column 8 of Table II, application no. 17, preferably a homolog or functional equivalent as depicted in column 8 of Table II B, application no. 17, and being depicted in the same respective line as said TTC1386, and preferably the activity is increased non-targeted, whereby the respective line disclose in column 7 the fine chemical fructose.

Accordingly, in one embodiment, the molecule which activity is to be increased in the process of the invention is the gene product with an activity as a “TTC1386-protein”, preferably it is the molecule of section (a) or (b) of this paragraph.

In particular, it was observed that in plants, especially in Arabidopsis thaliana, increasing or generating the activity of a gene product non-targeted with the activity of a “TTC1386-protein”, preferably being encoded by a gene comprising the nucleic acid sequence SEQ ID NO. 62079, preferably the coding region thereof, conferred the production of or the increase in fructose compared with the wild type control.

The nucleic acid sequence of TTC1386 from Thermus thermophilus, e.g. as shown in the respective line in column 5 of Table I, application no. 17, has been published in E. And the activity of the gene product thereof is the activity of TTC1386-protein.

Accordingly, in one embodiment, the process of the present invention for producing glucose in a non-human organism, like a microorganism or a plant or a part thereof, comprises increasing or generating the activity of a gene product with the activity of a gene product conferring the activity of “TTC1386-protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said TTC1386, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said TTC1386, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said TTC1386, or a functional equivalent or a homolog thereof as depicted in column 8 of Table II, application no. 17, preferably a homolog or functional equivalent as depicted in column 8 of Table II B, application no. 17, and being depicted in the same respective line as said TTC1386, and preferably the activity is increased non-targeted, whereby the respective line disclose in column 7 the fine chemical glucose.

Accordingly, in one embodiment, the molecule which activity is to be increased in the process of the invention is the gene product with an activity as a “TTC1386-protein”, preferably it is the molecule of section (a) or (b) of this paragraph.

In particular, it was observed that in plants, especially in Arabidopsis thaliana, increasing or generating the activity of a gene product non-targeted with the activity of a “TTC1386-protein”, preferably being encoded by a gene comprising the nucleic acid sequence SEQ ID NO. 62079, preferably the coding region thereof, conferred the production of or the increase in glucose compared with the wild type control.

The nucleic acid sequence of TTC1550 from Thermus thermophilus, e.g. as shown in the respective line in column 5 of Table I, application no. 17, has been published in E. And the activity of the gene product thereof is the activity of homocitrate synthase.

Accordingly, in one embodiment, the process of the present invention for producing fructose in a non-human organism, like a microorganism or a plant or a part thereof, comprises increasing or generating the activity of a gene product with the activity of a gene product conferring the activity of “homocitrate synthase”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said TTC1550, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said TTC1550, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said TTC1550, or a functional equivalent or a homolog thereof as depicted in column 8 of Table II, application no. 17, preferably a homolog or functional equivalent as depicted in column 8 of Table II B, application no. 17, and being depicted in the same respective line as said TTC1550, and preferably the activity is increased non-targeted, whereby the respective line disclose in column 7 the fine chemical fructose.

Accordingly, in one embodiment, the molecule which activity is to be increased in the process of the invention is the gene product with an activity as a “homocitrate synthase”, preferably it is the molecule of section (a) or (b) of this paragraph.

In particular, it was observed that in plants, especially in Arabidopsis thaliana, increasing or generating the activity of a gene product non-targeted with the activity of a “homocitrate synthase”, preferably being encoded by a gene comprising the nucleic acid sequence SEQ ID NO. 12974, preferably the coding region thereof, conferred the production of or the increase in fructose compared with the wild type control.

The nucleic acid sequence of TTC1550 from Thermus thermophilus, e.g. as shown in the respective line in column 5 of Table I, application no. 17, has been published in E. And the activity of the gene product thereof is the activity of homocitrate synthase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “homocitrate synthase”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said TTC1550, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said TTC1550, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said TTC1550, or a functional equivalent or a homolog thereof as depicted in column 8 of Table II, application no. 17, preferably a homolog or functional equivalent as depicted in column 8 of Table II B, application no. 17, and being depicted in the same respective line as said TTC1550, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “homocitrate synthase”, preferably it is the molecule of section (a) or (b) of this paragraph.

In particular, it was observed that in plants, especially in Arabidopsis thaliana, increasing or generating the activity of a gene product non-targeted with the activity of a “homocitrate synthase”, preferably being encoded by a gene comprising the nucleic acid sequence SEQ ID NO. 12974, preferably the coding region thereof, conferred the production of or the increase in glucose-1-phosphate compared with the wild type control.

The nucleic acid sequence of TTC1550 from Thermus thermophilus, e.g. as shown in the respective line in column 5 of Table I, application no. 17, has been published in E. And the activity of the gene product thereof is the activity of homocitrate synthase.

Accordingly, in one embodiment, the process of the present invention for producing sucrose in a non-human organism, like a microorganism or a plant or a part thereof, comprises increasing or generating the activity of a gene product with the activity of a gene product conferring the activity of “homocitrate synthase”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said TTC1550, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said TTC1550, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said TTC1550, or a functional equivalent or a homolog thereof as depicted in column 8 of Table II, application no. 17, preferably a homolog or functional equivalent as depicted in column 8 of Table II B, application no. 17, and being depicted in the same respective line as said TTC1550, and preferably the activity is increased non-targeted, whereby the respective line disclose in column 7 the fine chemical sucrose.

Accordingly, in one embodiment, the molecule which activity is to be increased in the process of the invention is the gene product with an activity as a “homocitrate synthase”, preferably it is the molecule of section (a) or (b) of this paragraph.

In particular, it was observed that in plants, especially in Arabidopsis thaliana, increasing or generating the activity of a gene product non-targeted with the activity of a “homocitrate synthase”, preferably being encoded by a gene comprising the nucleic acid sequence SEQ ID NO. 12974, preferably the coding region thereof, conferred the production of or the increase in sucrose compared with the wild type control.

The nucleic acid sequence of TTC1893 from Thermus thermophilus, e.g. as shown in the respective line in column 5 of Table I, application no. 17, has been published in E. And the activity of the gene product thereof is the activity of permease.

Accordingly, in one embodiment, the process of the present invention for producing raffinose in a non-human organism, like a microorganism or a plant or a part thereof, comprises increasing or generating the activity of a gene product with the activity of a gene product conferring the activity of “permease”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said TTC1893, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said TTC1893, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said TTC1893, or a functional equivalent or a homolog thereof as depicted in column 8 of Table II, application no. 17, preferably a homolog or functional equivalent as depicted in column 8 of Table II B, application no. 17, and being depicted in the same respective line as said TTC1893, and preferably the activity is increased non-targeted, whereby the respective line disclose in column 7 the fine chemical raffinose.

Accordingly, in one embodiment, the molecule which activity is to be increased in the process of the invention is the gene product with an activity as a “permease”, preferably it is the molecule of section (a) or (b) of this paragraph.

In particular, it was observed that in plants, especially in Arabidopsis thaliana, increasing or generating the activity of a gene product non-targeted with the activity of a “permease”, preferably being encoded by a gene comprising the nucleic acid sequence SEQ ID NO. 148559, preferably the coding region thereof, conferred the production of or the increase in raffinose compared with the wild type control.

The nucleic acid sequence of TTC1893 from Thermus thermophilus, e.g. as shown in the respective line in column 5 of Table I, application no. 17, has been published in E. And the activity of the gene product thereof is the activity of permease.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “permease”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said TTC1893, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said TTC1893, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said TTC1893, or a functional equivalent or a homolog thereof as depicted in column 8 of Table II, application no. 17, preferably a homolog or functional equivalent as depicted in column 8 of Table II B, application no. 17, and being depicted in the same respective line as said TTC1893, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “permease”, preferably it is the molecule of section (a) or (b) of this paragraph.

In particular, it was observed that in plants, especially in Arabidopsis thaliana, increasing or generating the activity of a gene product non-targeted with the activity of a “permease”, preferably being encoded by a gene comprising the nucleic acid sequence SEQ ID NO. 148559, preferably the coding region thereof, conferred the production of or the increase in 3,4,5-trihydroxypentanoic acid compared with the wild type control.

The nucleic acid sequence of TTC1918 from Thermus thermophilus, e.g. as shown in the respective line in column 5 of Table I, application no. 17, has been published in E. And the activity of the gene product thereof is the activity of oxidoreductase subunit.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “oxidoreductase subunit”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said TTC1918, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said TTC1918, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said TTC1918, or a functional equivalent or a homolog thereof as depicted in column 8 of Table II, application no. 17, preferably a homolog or functional equivalent as depicted in column 8 of Table II B, application no. 17, and being depicted in the same respective line as said TTC1918, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “oxidoreductase subunit”, preferably it is the molecule of section (a) or (b) of this paragraph.

In particular, it was observed that in plants, especially in Arabidopsis thaliana, increasing or generating the activity of a gene product non-targeted with the activity of a “oxidoreductase subunit”, preferably being encoded by a gene comprising the nucleic acid sequence SEQ ID NO. 62160, preferably the coding region thereof, conferred the production of or the increase in glucose-1-phosphate compared with the wild type control.

The nucleic acid sequence of XM_{—}473199 from Oryza sativa, e.g. as shown in the respective line in column 5 of Table I, application no. 17, has been published in G. And the activity of the gene product thereof is the activity of XM_{—}473199-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “XM_{—}473199-protein”, especially from Oryza sativa or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said XM_{—}473199, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said XM_{—}473199, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said XM_{—}473199, or a functional equivalent or a homolog thereof as depicted in column 8 of Table II, application no. 17, preferably a homolog or functional equivalent as depicted in column 8 of Table II B, application no. 17, and being depicted in the same respective line as said XM_{—}473199, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “XM_{—}473199-protein”, preferably it is the molecule of section (a) or (b) of this paragraph.

In particular, it was observed that in plants, especially in Arabidopsis thaliana, increasing or generating the activity of a gene product non-targeted with the activity of a “XM_{—}473199-protein”, preferably being encoded by a gene comprising the nucleic acid sequence SEQ ID NO. 62244, preferably the coding region thereof, conferred the production of or the increase in glucose-1-phosphate compared with the wild type control.

The nucleic acid sequence of Ybl021c from Saccharomyces cerevisiae, e.g. as shown in the respective line in column 5 of Table I, application no. 17, has been published in A. And the activity of the gene product thereof is the activity of transcription factor.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “transcription factor”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said Ybl021c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ybl021 c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ybl021c, or a functional equivalent or a homolog thereof as depicted in column 8 of Table II, application no. 17, preferably a homolog or functional equivalent as depicted in column 8 of Table II B, application no. 17, and being depicted in the same respective line as said Ybl021c, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 62524, preferably the coding region thereof, conferred the production of or the increase in myo-inositol compared with the wild type control.

The nucleic acid sequence of Ybl021 c from Saccharomyces cerevisiae, e.g. as shown in the respective line in column 5 of Table I, application no. 17, has been published in A. And the activity of the gene product thereof is the activity of transcription factor.

Accordingly, in one embodiment, the process of the present invention for producing sucrose in a non-human organism, like a microorganism or a plant or a part thereof, comprises increasing or generating the activity of a gene product with the activity of a gene product conferring the activity of “transcription factor”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said Ybl021 c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ybl021c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ybl021c, or a functional equivalent or a homolog thereof as depicted in column 8 of Table II, application no. 17, preferably a homolog or functional equivalent as depicted in column 8 of Table II B, application no. 17, and being depicted in the same respective line as said Ybl021c, and preferably the activity is increased non-targeted, whereby the respective line disclose in column 7 the fine chemical sucrose.

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 62524, preferably the coding region thereof, conferred the production of or the increase in sucrose compared with the wild type control.

The nucleic acid sequence of Ybr249c from Saccharomyces cerevisiae, e.g. as shown in the respective line in column 5 of Table I, application no. 17, has been published in A. And the activity of the gene product thereof is the activity of 3-deoxy-7-phosphoheptulonate synthase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “3-deoxy-7-phosphoheptulonate synthase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Ybr249c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ybr249c, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ybr249c, or a functional equivalent or a homolog thereof as depicted in column 8 of Table II, application no. 17, preferably a homolog or functional equivalent as depicted in column 8 of Table II B, application no. 17, and being depicted in the same respective line as said Ybr249c, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “3-deoxy-7-phosphoheptulonate synthase”, preferably it is the molecule of section (a) or (b) of this paragraph.

In particular, it was observed that in plants, especially in Arabidopsis thaliana, increasing or generating the activity of a gene product plastidic with the activity of a “3-deoxy-7-phosphoheptulonate synthase”, preferably being encoded by a gene comprising the nucleic acid sequence SEQ ID NO. 88879, preferably the coding region thereof, conferred the production of or the increase in glucose-1-phosphate compared with the wild type control.

The nucleic acid sequence of Ybr296c from Saccharomyces cerevisiae, e.g. as shown in the respective line in column 5 of Table I, application no. 17, has been published in A. And the activity of the gene product thereof is the activity of phosphate permease.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “phosphate permease”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Ybr296c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ybr296c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ybr296c, or a functional equivalent or a homolog thereof as depicted in column 8 of Table II, application no. 17, preferably a homolog or functional equivalent as depicted in column 8 of Table II B, application no. 17, and being depicted in the same respective line as said Ybr296c, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “phosphate permease”, preferably it is the molecule of section (a) or (b) of this paragraph.

In particular, it was observed that in plants, especially in Arabidopsis thaliana, increasing or generating the activity of a gene product non-targeted with the activity of a “phosphate permease”, preferably being encoded by a gene comprising the nucleic acid sequence SEQ ID NO. 89317, preferably the coding region thereof, conferred the production of or the increase in glucose-1-phosphate compared with the wild type control.

The nucleic acid sequence of Ybr296c from Saccharomyces cerevisiae, e.g. as shown in the respective line in column 5 of Table I, application no. 17, has been published in A. And the activity of the gene product thereof is the activity of phosphate permease.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “phosphate permease”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said Ybr296c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ybr296c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ybr296c, or a functional equivalent or a homolog thereof as depicted in column 8 of Table II, application no. 17, preferably a homolog or functional equivalent as depicted in column 8 of Table II B, application no. 17, and being depicted in the same respective line as said Ybr296c, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “phosphate permease”, preferably it is the molecule of section (a) or (b) of this paragraph.

In particular, it was observed that in plants, especially in Arabidopsis thaliana, increasing or generating the activity of a gene product non-targeted with the activity of a “phosphate permease”, preferably being encoded by a gene comprising the nucleic acid sequence SEQ ID NO. 89317, preferably the coding region thereof, conferred the production of or the increase in 3,4,5-trihydroxypentanoic acid compared with the wild type control.

The nucleic acid sequence of Ycl027w from Saccharomyces cerevisiae, e.g. as shown in the respective line in column 5 of Table I, application no. 17, has been published in A. And the activity of the gene product thereof is the activity of ycl027w-protein.

Accordingly, in one embodiment, the process of the present invention for producing fructose in a non-human organism, like a microorganism or a plant or a part thereof, comprises increasing or generating the activity of a gene product with the activity of a gene product conferring the activity of “ycl027w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said Ycl027w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ycl027w, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ycl027w, or a functional equivalent or a homolog thereof as depicted in column 8 of Table II, application no. 17, preferably a homolog or functional equivalent as depicted in column 8 of Table II B, application no. 17, and being depicted in the same respective line as said Ycl027w, and preferably the activity is increased plastidic, whereby the respective line disclose in column 7 the fine chemical fructose.

Accordingly, in one embodiment, the molecule which activity is to be increased in the process of the invention is the gene product with an activity as a “ycl027w-protein”, preferably it is the molecule of section (a) or (b) of this paragraph.

In particular, it was observed that in plants, especially in Arabidopsis thaliana, increasing or generating the activity of a gene product plastidic with the activity of a “ycl027w-protein”, preferably being encoded by a gene comprising the nucleic acid sequence SEQ ID NO. 96980, preferably the coding region thereof, conferred the production of or the increase in fructose compared with the wild type control.

The nucleic acid sequence of Ycl027w from Saccharomyces cerevisiae, e.g. as shown in the respective line in column 5 of Table I, application no. 17, has been published in A. And the activity of the gene product thereof is the activity of ycl027w-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “ycl027w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Ycl027w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ycl027w, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ycl027w, or a functional equivalent or a homolog thereof as depicted in column 8 of Table II, application no. 17, preferably a homolog or functional equivalent as depicted in column 8 of Table II B, application no. 17, and being depicted in the same respective line as said Ycl027w, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “ycl027w-protein”, preferably it is the molecule of section (a) or (b) of this paragraph.

In particular, it was observed that in plants, especially in Arabidopsis thaliana, increasing or generating the activity of a gene product plastidic with the activity of a “ycl027w-protein”, preferably being encoded by a gene comprising the nucleic acid sequence SEQ ID NO. 96980, preferably the coding region thereof, conferred the production of or the increase in glucose-1-phosphate compared with the wild type control.

The nucleic acid sequence of Ycl064c from Saccharomyces cerevisiae, e.g. as shown in the respective line in column 5 of Table I, application no. 17, has been published in A. And the activity of the gene product thereof is the activity of serine/threonine dehydratase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “serine/threonine dehydratase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said Ycl064c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ycl064c, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ycl064c, or a functional equivalent or a homolog thereof as depicted in column 8 of Table II, application no. 17, preferably a homolog or functional equivalent as depicted in column 8 of Table II B, application no. 17, and being depicted in the same respective line as said Ycl064c, and preferably the activity is increased plastidic, whereby the respective line disclose in column 7 the fine chemical myo-inositol.

Accordingly, in one embodiment, the molecule which activity is to be increased in the process of the invention is the gene product with an activity as a “serine/threonine dehydratase”, preferably it is the molecule of section (a) or (b) of this paragraph.

In particular, it was observed that in plants, especially in Arabidopsis thaliana, increasing or generating the activity of a gene product plastidic with the activity of a “serine/threonine dehydratase”, preferably being encoded by a gene comprising the nucleic acid sequence SEQ ID NO. 89395, preferably the coding region thereof, conferred the production of or the increase in myo-inositol compared with the wild type control.

The nucleic acid sequence of Ycl064c from Saccharomyces cerevisiae, e.g. as shown in the respective line in column 5 of Table I, application no. 17, has been published in A. And the activity of the gene product thereof is the activity of serine/threonine dehydratase.

Accordingly, in one embodiment, the process of the present invention for producing sucrose in a non-human organism, like a microorganism or a plant or a part thereof, comprises increasing or generating the activity of a gene product with the activity of a gene product conferring the activity of “serine/threonine dehydratase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said Ycl064c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ycl064c, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ycl064c, or a functional equivalent or a homolog thereof as depicted in column 8 of Table II, application no. 17, preferably a homolog or functional equivalent as depicted in column 8 of Table II B, application no. 17, and being depicted in the same respective line as said Ycl064c, and preferably the activity is increased plastidic, whereby the respective line disclose in column 7 the fine chemical sucrose.

Accordingly, in one embodiment, the molecule which activity is to be increased in the process of the invention is the gene product with an activity as a “serine/threonine dehydratase”, preferably it is the molecule of section (a) or (b) of this paragraph.

In particular, it was observed that in plants, especially in Arabidopsis thaliana, increasing or generating the activity of a gene product plastidic with the activity of a “serine/threonine dehydratase”, preferably being encoded by a gene comprising the nucleic acid sequence SEQ ID NO. 89395, preferably the coding region thereof, conferred the production of or the increase in sucrose compared with the wild type control.

The nucleic acid sequence of Ycr012w from Saccharomyces cerevisiae, e.g. as shown in the respective line in column 5 of Table I, application no. 17, has been published in A. And the activity of the gene product thereof is the activity of phosphoglycerate kinase.

Accordingly, in one embodiment, the process of the present invention for producing fructose in a non-human organism, like a microorganism or a plant or a part thereof, comprises increasing or generating the activity of a gene product with the activity of a gene product conferring the activity of “phosphoglycerate kinase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said Ycr012w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ycr012w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ycr012w, or a functional equivalent or a homolog thereof as depicted in column 8 of Table II, application no. 17, preferably a homolog or functional equivalent as depicted in column 8 of Table II B, application no. 17, and being depicted in the same respective line as said Ycr012w, and preferably the activity is increased non-targeted, whereby the respective line disclose in column 7 the fine chemical fructose.

Accordingly, in one embodiment, the molecule which activity is to be increased in the process of the invention is the gene product with an activity as a “phosphoglycerate kinase”, preferably it is the molecule of section (a) or (b) of this paragraph.

In particular, it was observed that in plants, especially in Arabidopsis thaliana, increasing or generating the activity of a gene product non-targeted with the activity of a “phosphoglycerate kinase”, preferably being encoded by a gene comprising the nucleic acid sequence SEQ ID NO. 108113, preferably the coding region thereof, conferred the production of or the increase in fructose compared with the wild type control.

The nucleic acid sequence of Ycr012w from Saccharomyces cerevisiae, e.g. as shown in the respective line in column 5 of Table I, application no. 17, has been published in A. And the activity of the gene product thereof is the activity of phosphoglycerate kinase.

Accordingly, in one embodiment, the process of the present invention for producing glucose in a non-human organism, like a microorganism or a plant or a part thereof, comprises increasing or generating the activity of a gene product with the activity of a gene product conferring the activity of “phosphoglycerate kinase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said Ycr012w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ycr012w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ycr012w, or a functional equivalent or a homolog thereof as depicted in column 8 of Table II, application no. 17, preferably a homolog or functional equivalent as depicted in column 8 of Table II B, application no. 17, and being depicted in the same respective line as said Ycr012w, and preferably the activity is increased non-targeted, whereby the respective line disclose in column 7 the fine chemical glucose.

Accordingly, in one embodiment, the molecule which activity is to be increased in the process of the invention is the gene product with an activity as a “phosphoglycerate kinase”, preferably it is the molecule of section (a) or (b) of this paragraph.

In particular, it was observed that in plants, especially in Arabidopsis thaliana, increasing or generating the activity of a gene product non-targeted with the activity of a “phosphoglycerate kinase”, preferably being encoded by a gene comprising the nucleic acid sequence SEQ ID NO. 108113, preferably the coding region thereof, conferred the production of or the increase in glucose compared with the wild type control.

The nucleic acid sequence of Ycr047w-a from Saccharomyces cerevisiae, e.g. as shown in the respective line in column 5 of Table I, application no. 17, has been published in A. And the activity of the gene product thereof is the activity of YCR047W-A-protein.

Accordingly, in one embodiment, the process of the present invention for producing raffinose in a non-human organism, like a microorganism or a plant or a part thereof, comprises increasing or generating the activity of a gene product with the activity of a gene product conferring the activity of “YCR047W-A-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said Ycr047w-a, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ycr047w-a, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ycr047w-a, or a functional equivalent or a homolog thereof as depicted in column 8 of Table II, application no. 17, preferably a homolog or functional equivalent as depicted in column 8 of Table II B, application no. 17, and being depicted in the same respective line as said Ycr047w-a, and preferably the activity is increased non-targeted, whereby the respective line disclose in column 7 the fine chemical raffinose.

Accordingly, in one embodiment, the molecule which activity is to be increased in the process of the invention is the gene product with an activity as a “YCR047W-A-protein”, preferably it is the molecule of section (a) or (b) of this paragraph.

In particular, it was observed that in plants, especially in Arabidopsis thaliana, increasing or generating the activity of a gene product non-targeted with the activity of a “YCR047W-A-protein”, preferably being encoded by a gene comprising the nucleic acid sequence SEQ ID NO. 148579, preferably the coding region thereof, conferred the production of or the increase in raffinose compared with the wild type control.

The nucleic acid sequence of Ycr102c from Saccharomyces cerevisiae, e.g. as shown in the respective line in column 5 of Table I, application no. 17, has been published in A. And the activity of the gene product thereof is the activity of ycr102c-protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “ycr102c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said Ycr102c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ycr102c, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ycr102c, or a functional equivalent or a homolog thereof as depicted in column 8 of Table II, application no. 17, preferably a homolog or functional equivalent as depicted in column 8 of Table II B, application no. 17, and being depicted in the same respective line as said Ycr102c, and preferably the activity is increased plastidic, whereby the respective line disclose in column 7 the fine chemical 3,4,5-trihydroxypentanoic acid.

Accordingly, in one embodiment, the molecule which activity is to be increased in the process of the invention is the gene product with an activity as a “ycr102c-protein”, preferably it is the molecule of section (a) or (b) of this paragraph.

In particular, it was observed that in plants, especially in Arabidopsis thaliana, increasing or generating the activity of a gene product plastidic with the activity of a “ycr102c-protein”, preferably being encoded by a gene comprising the nucleic acid sequence SEQ ID NO. 63264, preferably the coding region thereof, conferred the production of or the increase in 3,4,5-trihydroxypentanoic acid compared with the wild type control.

The nucleic acid sequence of Ydl066w from Saccharomyces cerevisiae, e.g. as shown in the respective line in column 5 of Table I, application no. 17, has been published in A. And the activity of the gene product thereof is the activity of isocitrate dehydrogenase.

Accordingly, in one embodiment, the process of the present invention for producing galactinol in a non-human organism, like a microorganism or a plant or a part thereof, comprises increasing or generating the activity of a gene product with the activity of a gene product conferring the activity of “isocitrate dehydrogenase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical galactinol), application no. 17, preferably the coding region thereof, or a homolog or a fragment thereof, and being depicted in the same respective line as said Ydl066w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ydl066w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ydl066w, or a functional equivalent or a homolog thereof as depicted in column 8 of Table II, application no. 17, preferably a homolog or functional equivalent as depicted in column 8 of Table II B, application no. 17, and being depicted in the same respective line as said Ydl066w, and preferably the activity is increased non-targeted, whereby the respective line disclose in column 7 the fine chemical galactinol.

Accordingly, in one embodiment, the molecule which activity is to be increased in the process of the invention is the gene product with an activity as a “isocitrate dehydrogenase”, preferably it is the molecule of section (a) or (b) of this paragraph.

In particular, it was observed that in plants, especially in Arabidopsis thaliana, increasing or generating the activity of a gene product non-targeted with the activity of a “isocitrate dehydrogenase”, preferably being encoded by a gene comprising the nucleic acid sequence SEQ ID NO. 122228, preferably the coding region thereof, conferred the production of or the increase in galactinol compared with the wild type control.

The nucleic acid sequence of Ydl126c from Saccharomyces cerevisiae, e.g. as shown in the respective line in column 5 of Table I, application no. 17, has been published in A. And the activity of the gene product thereof is the activity of cell division control protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “cell division control protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said Ydl126c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ydl126c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ydl126c, or a functional equivalent or a homolog thereof as depicted in column 8 of Table II, application no. 17, preferably a homolog or functional equivalent as depicted in column 8 of Table II B, application no. 17, and being depicted in the same respective line as said Ydl126c, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “cell division control protein”, preferably it is the molecule of section (a) or (b) of this paragraph.

In particular, it was observed that in plants, especially in Arabidopsis thaliana, increasing or generating the activity of a gene product non-targeted with the activity of a “cell division control protein”, preferably being encoded by a gene comprising the nucleic acid sequence SEQ ID NO. 120045, preferably the coding region thereof, conferred the production of or the increase in myo-inositol compared with the wild type control.

The nucleic acid sequence of Ydl155w from Saccharomyces cerevisiae, e.g. as shown in the respective line in column 5 of Table I, application no. 17, has been published in A. And the activity of the gene product thereof is the activity of cyclin.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “cyclin”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said Ydl155w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ydl155w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ydl155w, or a functional equivalent or a homolog thereof as depicted in column 8 of Table II, application no. 17, preferably a homolog or functional equivalent as depicted in column 8 of Table II B, application no. 17, and being depicted in the same respective line as said Ydl155w, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “cyclin”, preferably it is the molecule of section (a) or (b) of this paragraph.

In particular, it was observed that in plants, especially in Arabidopsis thaliana, increasing or generating the activity of a gene product non-targeted with the activity of a “cyclin”, preferably being encoded by a gene comprising the nucleic acid sequence SEQ ID NO. 14171, preferably the coding region thereof, conferred the production of or the increase in myo-inositol compared with the wild type control.

The nucleic acid sequence of Ydl159w-a from Saccharomyces cerevisiae, e.g. as shown in the respective line in column 5 of Table I, application no. 17, has been published in A. And the activity of the gene product thereof is the activity of YDL159W-A-protein.

Accordingly, in one embodiment, the process of the present invention for producing raffinose in a non-human organism, like a microorganism or a plant or a part thereof, comprises increasing or generating the activity of a gene product with the activity of a gene product conferring the activity of “YDL159W-A-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said Ydl159w-a, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ydl159w-a, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ydl159w-a, or a functional equivalent or a homolog thereof as depicted in column 8 of Table II, application no. 17, preferably a homolog or functional equivalent as depicted in column 8 of Table II B, application no. 17, and being depicted in the same respective line as said Ydl159w-a, and preferably the activity is increased non-targeted, whereby the respective line disclose in column 7 the fine chemical raffinose.

Accordingly, in one embodiment, the molecule which activity is to be increased in the process of the invention is the gene product with an activity as a “YDL159W-A-protein”, preferably it is the molecule of section (a) or (b) of this paragraph.

In particular, it was observed that in plants, especially in Arabidopsis thaliana, increasing or generating the activity of a gene product non-targeted with the activity of a “YDL159W-A-protein”, preferably being encoded by a gene comprising the nucleic acid sequence SEQ ID NO. 137498, preferably the coding region thereof, conferred the production of or the increase in raffinose compared with the wild type control.

The nucleic acid sequence of Ydl168w from Saccharomyces cerevisiae, e.g. as shown in the respective line in column 5 of Table I, application no. 17, has been published in A. And the activity of the gene product thereof is the activity of protein kinase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “protein kinase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Ydl168w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ydl168w, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ydl168w, or a functional equivalent or a homolog thereof as depicted in column 8 of Table II, application no. 17, preferably a homolog or functional equivalent as depicted in column 8 of Table II B, application no. 17, and being depicted in the same respective line as said Ydl168w, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product plastidic with the activity of a “protein kinase”, preferably being encoded by a gene comprising the nucleic acid sequence SEQ ID NO. 14302, preferably the coding region thereof, conferred the production of or the increase in glucose-1-phosphate compared with the wild type control.

The nucleic acid sequence of Ydl188c from Saccharomyces cerevisiae, e.g. as shown in the respective line in column 5 of Table I, application no. 17, has been published in A. And the activity of the gene product thereof is the activity of serine/threonine-protein phosphatase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “serine/threonine-protein phosphatase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Ydl188c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ydl188c, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ydl188c, or a functional equivalent or a homolog thereof as depicted in column 8 of Table II, application no. 17, preferably a homolog or functional equivalent as depicted in column 8 of Table II B, application no. 17, and being depicted in the same respective line as said Ydl188c, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “serine/threonine-protein phosphatase”, preferably it is the molecule of section (a) or (b) of this paragraph.

In particular, it was observed that in plants, especially in Arabidopsis thaliana, increasing or generating the activity of a gene product plastidic with the activity of a “serine/threonine-protein phosphatase”, preferably being encoded by a gene comprising the nucleic acid sequence SEQ ID NO. 89483, preferably the coding region thereof, conferred the production of or the increase in glucose-1-phosphate compared with the wild type control.

The nucleic acid sequence of YDL222C from Saccharomyces cerevisiae, e.g. as shown in the respective line in column 5 of Table I, application no. 17, has been published in A. And the activity of the gene product thereof is the activity of integral membrane protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “integral membrane protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said YDL222C, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said YDL222C, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said YDL222C, or a functional equivalent or a homolog thereof as depicted in column 8 of Table II, application no. 17, preferably a homolog or functional equivalent as depicted in column 8 of Table II B, application no. 17, and being depicted in the same respective line as said YDL222C, and preferably the activity is increased plastidic, whereby the respective line disclose in column 7 the fine chemical 3,4,5-trihydroxypentanoic acid.

Accordingly, in one embodiment, the molecule which activity is to be increased in the process of the invention is the gene product with an activity as a “integral membrane protein”, preferably it is the molecule of section (a) or (b) of this paragraph.

In particular, it was observed that in plants, especially in Arabidopsis thaliana, increasing or generating the activity of a gene product plastidic with the activity of a “integral membrane protein”, preferably being encoded by a gene comprising the nucleic acid sequence SEQ ID NO. 148583, preferably the coding region thereof, conferred the production of or the increase in 3,4,5-trihydroxypentanoic acid compared with the wild type control.

The nucleic acid sequence of Ydl235c from Saccharomyces cerevisiae, e.g. as shown in the respective line in column 5 of Table I, application no. 17, has been published in A. And the activity of the gene product thereof is the activity of ydl235c-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “ydl235c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Ydl235c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ydl235c, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ydl235c, or a functional equivalent or a homolog thereof as depicted in column 8 of Table II, application no. 17, preferably a homolog or functional equivalent as depicted in column 8 of Table II B, application no. 17, and being depicted in the same respective line as said Ydl235c, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “ydl235c-protein”, preferably it is the molecule of section (a) or (b) of this paragraph.

In particular, it was observed that in plants, especially in Arabidopsis thaliana, increasing or generating the activity of a gene product plastidic with the activity of a “ydl235c-protein”, preferably being encoded by a gene comprising the nucleic acid sequence SEQ ID NO. 113313, preferably the coding region thereof, conferred the production of or the increase in glucose-1-phosphate compared with the wild type control.

The nucleic acid sequence of Ydl235c from Saccharomyces cerevisiae, e.g. as shown in the respective line in column 5 of Table I, application no. 17, has been published in A. And the activity of the gene product thereof is the activity of ydl235c-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “ydl235c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said Ydl235c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ydl235c, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ydl235c, or a functional equivalent or a homolog thereof as depicted in column 8 of Table II, application no. 17, preferably a homolog or functional equivalent as depicted in column 8 of Table II B, application no. 17, and being depicted in the same respective line as said Ydl235c, and preferably the activity is increased plastidic, whereby the respective line disclose in column 7 the fine chemical myo-inositol.

Accordingly, in one embodiment, the molecule which activity is to be increased in the process of the invention is the gene product with an activity as a “ydl235c-protein”, preferably it is the molecule of section (a) or (b) of this paragraph.

In particular, it was observed that in plants, especially in Arabidopsis thaliana, increasing or generating the activity of a gene product plastidic with the activity of a “ydl235c-protein”, preferably being encoded by a gene comprising the nucleic acid sequence SEQ ID NO. 113313, preferably the coding region thereof, conferred the production of or the increase in myo-inositol compared with the wild type control.

The nucleic acid sequence of Ydl235c from Saccharomyces cerevisiae, e.g. as shown in the respective line in column 5 of Table I, application no. 17, has been published in A. And the activity of the gene product thereof is the activity of ydl235c-protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “ydl235c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said Ydl235c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ydl235c, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ydl235c, or a functional equivalent or a homolog thereof as depicted in column 8 of Table II, application no. 17, preferably a homolog or functional equivalent as depicted in column 8 of Table II B, application no. 17, and being depicted in the same respective line as said Ydl235c, and preferably the activity is increased plastidic, whereby the respective line disclose in column 7 the fine chemical 3,4,5-trihydroxypentanoic acid.

Accordingly, in one embodiment, the molecule which activity is to be increased in the process of the invention is the gene product with an activity as a “ydl235c-protein”, preferably it is the molecule of section (a) or (b) of this paragraph.

In particular, it was observed that in plants, especially in Arabidopsis thaliana, increasing or generating the activity of a gene product plastidic with the activity of a “ydl235c-protein”, preferably being encoded by a gene comprising the nucleic acid sequence SEQ ID NO. 113313, preferably the coding region thereof, conferred the production of or the increase in 3,4,5-trihydroxypentanoic acid compared with the wild type control.

The nucleic acid sequence of Ydr044w from Saccharomyces cerevisiae, e.g. as shown in the respective line in column 5 of Table I, application no. 17, has been published in A. And the activity of the gene product thereof is the activity of coproporphyrinogen III oxidase.

Accordingly, in one embodiment, the process of the present invention for producing sucrose in a non-human organism, like a microorganism or a plant or a part thereof, comprises increasing or generating the activity of a gene product with the activity of a gene product conferring the activity of “coproporphyrinogen III oxidase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said Ydr044w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ydr044w, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ydr044w, or a functional equivalent or a homolog thereof as depicted in column 8 of Table II, application no. 17, preferably a homolog or functional equivalent as depicted in column 8 of Table II B, application no. 17, and being depicted in the same respective line as said Ydr044w, and preferably the activity is increased plastidic, whereby the respective line disclose in column 7 the fine chemical sucrose.

Accordingly, in one embodiment, the molecule which activity is to be increased in the process of the invention is the gene product with an activity as a “coproporphyrinogen III oxidase”, preferably it is the molecule of section (a) or (b) of this paragraph.

In particular, it was observed that in plants, especially in Arabidopsis thaliana, increasing or generating the activity of a gene product plastidic with the activity of a “coproporphyrinogen III oxidase”, preferably being encoded by a gene comprising the nucleic acid sequence SEQ ID NO. 63334, preferably the coding region thereof, conferred the production of or the increase in sucrose compared with the wild type control.

The nucleic acid sequence of Ydr046c from Saccharomyces cerevisiae, e.g. as shown in the respective line in column 5 of Table I, application no. 17, has been published in A. And the activity of the gene product thereof is the activity of branched-chain amino acid permease.

Accordingly, in one embodiment, the process of the present invention for producing glucose in a non-human organism, like a microorganism or a plant or a part thereof, comprises increasing or generating the activity of a gene product with the activity of a gene product conferring the activity of “branched-chain amino acid permease”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said Ydr046c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ydr046c, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ydr046c, or a functional equivalent or a homolog thereof as depicted in column 8 of Table II, application no. 17, preferably a homolog or functional equivalent as depicted in column 8 of Table II B, application no. 17, and being depicted in the same respective line as said Ydr046c, and preferably the activity is increased plastidic, whereby the respective line disclose in column 7 the fine chemical glucose.

Accordingly, in one embodiment, the molecule which activity is to be increased in the process of the invention is the gene product with an activity as a “branched-chain amino acid permease”, preferably it is the molecule of section (a) or (b) of this paragraph.

In particular, it was observed that in plants, especially in Arabidopsis thaliana, increasing or generating the activity of a gene product plastidic with the activity of a “branched-chain amino acid permease”, preferably being encoded by a gene comprising the nucleic acid sequence SEQ ID NO. 63544, preferably the coding region thereof, conferred the production of or the increase in glucose compared with the wild type control.

The nucleic acid sequence of Ydr183w from Saccharomyces cerevisiae, e.g. as shown in the respective line in column 5 of Table I, application no. 17, has been published in A. And the activity of the gene product thereof is the activity of ydr183w-protein.

Accordingly, in one embodiment, the process of the present invention for producing fructose in a non-human organism, like a microorganism or a plant or a part thereof, comprises increasing or generating the activity of a gene product with the activity of a gene product conferring the activity of “ydr183w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said Ydr183w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ydr183w, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ydr183w, or a functional equivalent or a homolog thereof as depicted in column 8 of Table II, application no. 17, preferably a homolog or functional equivalent as depicted in column 8 of Table II B, application no. 17, and being depicted in the same respective line as said Ydr183w, and preferably the activity is increased plastidic, whereby the respective line disclose in column 7 the fine chemical fructose.

Accordingly, in one embodiment, the molecule which activity is to be increased in the process of the invention is the gene product with an activity as a “ydr183w-protein”, preferably it is the molecule of section (a) or (b) of this paragraph.

In particular, it was observed that in plants, especially in Arabidopsis thaliana, increasing or generating the activity of a gene product plastidic with the activity of a “ydr183w-protein”, preferably being encoded by a gene comprising the nucleic acid sequence SEQ ID NO. 90103, preferably the coding region thereof, conferred the production of or the increase in fructose compared with the wild type control.

The nucleic acid sequence of Ydr183w from Saccharomyces cerevisiae, e.g. as shown in the respective line in column 5 of Table I, application no. 17, has been published in A. And the activity of the gene product thereof is the activity of ydr183w-protein.

Accordingly, in one embodiment, the process of the present invention for producing glucose in a non-human organism, like a microorganism or a plant or a part thereof, comprises increasing or generating the activity of a gene product with the activity of a gene product conferring the activity of “ydr183w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said Ydr183w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ydr183w, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ydr183w, or a functional equivalent or a homolog thereof as depicted in column 8 of Table II, application no. 17, preferably a homolog or functional equivalent as depicted in column 8 of Table II B, application no. 17, and being depicted in the same respective line as said Ydr183w, and preferably the activity is increased plastidic, whereby the respective line disclose in column 7 the fine chemical glucose.

Accordingly, in one embodiment, the molecule which activity is to be increased in the process of the invention is the gene product with an activity as a “ydr183w-protein”, preferably it is the molecule of section (a) or (b) of this paragraph.

In particular, it was observed that in plants, especially in Arabidopsis thaliana, increasing or generating the activity of a gene product plastidic with the activity of a “ydr183w-protein”, preferably being encoded by a gene comprising the nucleic acid sequence SEQ ID NO. 90103, preferably the coding region thereof, conferred the production of or the increase in glucose compared with the wild type control.

The nucleic acid sequence of Ydr507c from Saccharomyces cerevisiae, e.g. as shown in the respective line in column 5 of Table I, application no. 17, has been published in A. And the activity of the gene product thereof is the activity of ydr507c-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “ydr507c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Ydr507c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ydr507c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ydr507c, or a functional equivalent or a homolog thereof as depicted in column 8 of Table II, application no. 17, preferably a homolog or functional equivalent as depicted in column 8 of Table II B, application no. 17, and being depicted in the same respective line as said Ydr507c, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “ydr507c-protein”, preferably it is the molecule of section (a) or (b) of this paragraph.

In particular, it was observed that in plants, especially in Arabidopsis thaliana, increasing or generating the activity of a gene product non-targeted with the activity of a “ydr507c-protein”, preferably being encoded by a gene comprising the nucleic acid sequence SEQ ID NO. 90165, preferably the coding region thereof, conferred the production of or the increase in glucose-1-phosphate compared with the wild type control.

The nucleic acid sequence of Yel011w from Saccharomyces cerevisiae, e.g. as shown in the respective line in column 5 of Table I, application no. 17, has been published in A. And the activity of the gene product thereof is the activity of 1,4-alpha-glucan branching enzyme.

Accordingly, in one embodiment, the process of the present invention for producing fructose in a non-human organism, like a microorganism or a plant or a part thereof, comprises increasing or generating the activity of a gene product with the activity of a gene product conferring the activity of “1,4-alpha-glucan branching enzyme”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said Yel011w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Yel011w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Yel011w, or a functional equivalent or a homolog thereof as depicted in column 8 of Table II, application no. 17, preferably a homolog or functional equivalent as depicted in column 8 of Table II B, application no. 17, and being depicted in the same respective line as said Yel011w, and preferably the activity is increased non-targeted, whereby the respective line disclose in column 7 the fine chemical fructose.

Accordingly, in one embodiment, the molecule which activity is to be increased in the process of the invention is the gene product with an activity as a “1,4-alpha-glucan branching enzyme”, preferably it is the molecule of section (a) or (b) of this paragraph.

In particular, it was observed that in plants, especially in Arabidopsis thaliana, increasing or generating the activity of a gene product non-targeted with the activity of a “1,4-alpha-glucan branching enzyme”, preferably being encoded by a gene comprising the nucleic acid sequence SEQ ID NO. 148614, preferably the coding region thereof, conferred the production of or the increase in fructose compared with the wild type control.

The nucleic acid sequence of Yel011w from Saccharomyces cerevisiae, e.g. as shown in the respective line in column 5 of Table I, application no. 17, has been published in A. And the activity of the gene product thereof is the activity of 1,4-alpha-glucan branching enzyme.

Accordingly, in one embodiment, the process of the present invention for producing glucose in a non-human organism, like a microorganism or a plant or a part thereof, comprises increasing or generating the activity of a gene product with the activity of a gene product conferring the activity of “1,4-alpha-glucan branching enzyme”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said Yel011w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Yel011w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Yel011w, or a functional equivalent or a homolog thereof as depicted in column 8 of Table II, application no. 17, preferably a homolog or functional equivalent as depicted in column 8 of Table II B, application no. 17, and being depicted in the same respective line as said Yel011w, and preferably the activity is increased non-targeted, whereby the respective line disclose in column 7 the fine chemical glucose.

Accordingly, in one embodiment, the molecule which activity is to be increased in the process of the invention is the gene product with an activity as a “1,4-alpha-glucan branching enzyme”, preferably it is the molecule of section (a) or (b) of this paragraph.

In particular, it was observed that in plants, especially in Arabidopsis thaliana, increasing or generating the activity of a gene product non-targeted with the activity of a “1,4-alpha-glucan branching enzyme”, preferably being encoded by a gene comprising the nucleic acid sequence SEQ ID NO. 148614, preferably the coding region thereof, conferred the production of or the increase in glucose compared with the wild type control.

The nucleic acid sequence of Yel030c-a from Saccharomyces cerevisiae, e.g. as shown in the respective line in column 5 of Table I, application no. 17, has been published in A. And the activity of the gene product thereof is the activity of YEL030C-A-protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “YEL030C-A-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said Yel030c-a, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Yel030c-a, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Yel030c-a, or a functional equivalent or a homolog thereof as depicted in column 8 of Table II, application no. 17, preferably a homolog or functional equivalent as depicted in column 8 of Table II B, application no. 17, and being depicted in the same respective line as said Yel030c-a, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “YEL030C-A-protein”, preferably it is the molecule of section (a) or (b) of this paragraph.

In particular, it was observed that in plants, especially in Arabidopsis thaliana, increasing or generating the activity of a gene product non-targeted with the activity of a “YEL030C-A-protein”, preferably being encoded by a gene comprising the nucleic acid sequence SEQ ID NO. 97294, preferably the coding region thereof, conferred the production of or the increase in 3,4,5-trihydroxypentanoic acid compared with the wild type control.

The nucleic acid sequence of Yel046c from Saccharomyces cerevisiae, e.g. as shown in the respective line in column 5 of Table I, application no. 17, has been published in A. And the activity of the gene product thereof is the activity of aldolase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “aldolase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Yel046c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Yel046c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Yel046c, or a functional equivalent or a homolog thereof as depicted in column 8 of Table II, application no. 17, preferably a homolog or functional equivalent as depicted in column 8 of Table II B, application no. 17, and being depicted in the same respective line as said Yel046c, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “aldolase”, preferably it is the molecule of section (a) or (b) of this paragraph.

In particular, it was observed that in plants, especially in Arabidopsis thaliana, increasing or generating the activity of a gene product non-targeted with the activity of a “aldolase”, preferably being encoded by a gene comprising the nucleic acid sequence SEQ ID NO. 128511, preferably the coding region thereof, conferred the production of or the increase in glucose-1-phosphate compared with the wild type control.

The nucleic acid sequence of Yer024w from Saccharomyces cerevisiae, e.g. as shown in the respective line in column 5 of Table I, application no. 17, has been published in A. And the activity of the gene product thereof is the activity of O-acetyltransferase.

Accordingly, in one embodiment, the process of the present invention for producing glucose in a non-human organism, like a microorganism or a plant or a part thereof, comprises increasing or generating the activity of a gene product with the activity of a gene product conferring the activity of “O-acetyltransferase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said Yer024w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Yer024w, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Yer024w, or a functional equivalent or a homolog thereof as depicted in column 8 of Table II, application no. 17, preferably a homolog or functional equivalent as depicted in column 8 of Table II B, application no. 17, and being depicted in the same respective line as said Yer024w, and preferably the activity is increased plastidic, whereby the respective line disclose in column 7 the fine chemical glucose.

Accordingly, in one embodiment, the molecule which activity is to be increased in the process of the invention is the gene product with an activity as a “O-acetyltransferase”, preferably it is the molecule of section (a) or (b) of this paragraph.

In particular, it was observed that in plants, especially in Arabidopsis thaliana, increasing or generating the activity of a gene product plastidic with the activity of a “O-acetyltransferase”, preferably being encoded by a gene comprising the nucleic acid sequence SEQ ID NO. 148811, preferably the coding region thereof, conferred the production of or the increase in glucose compared with the wild type control.

The nucleic acid sequence of Yer177w from Saccharomyces cerevisiae, e.g. as shown in the respective line in column 5 of Table I, application no. 17, has been published in A. And the activity of the gene product thereof is the activity of yer177w-protein.

Accordingly, in one embodiment, the process of the present invention for producing raffinose in a non-human organism, like a microorganism or a plant or a part thereof, comprises increasing or generating the activity of a gene product with the activity of a gene product conferring the activity of “yer177w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said Yer177w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Yer177w, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Yer177w, or a functional equivalent or a homolog thereof as depicted in column 8 of Table II, application no. 17, preferably a homolog or functional equivalent as depicted in column 8 of Table II B, application no. 17, and being depicted in the same respective line as said Yer177w, and preferably the activity is increased plastidic, whereby the respective line disclose in column 7 the fine chemical raffinose.

Accordingly, in one embodiment, the molecule which activity is to be increased in the process of the invention is the gene product with an activity as a “yer177w-protein”, preferably it is the molecule of section (a) or (b) of this paragraph.

In particular, it was observed that in plants, especially in Arabidopsis thaliana, increasing or generating the activity of a gene product plastidic with the activity of a “yer177w-protein”, preferably being encoded by a gene comprising the nucleic acid sequence SEQ ID NO. 148833, preferably the coding region thereof, conferred the production of or the increase in raffinose compared with the wild type control.

The nucleic acid sequence of Yfl019c from Saccharomyces cerevisiae, e.g. as shown in the respective line in column 5 of Table I, application no. 17, has been published in A. And the activity of the gene product thereof is the activity of YFL019C-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “YFL019C-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said Yfl019c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Yfl019c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Yfl019c, or a functional equivalent or a homolog thereof as depicted in column 8 of Table II, application no. 17, preferably a homolog or functional equivalent as depicted in column 8 of Table II B, application no. 17, and being depicted in the same respective line as said Yfl019c, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “YFL0190-protein”, preferably it is the molecule of section (a) or (b) of this paragraph.

In particular, it was observed that in plants, especially in Arabidopsis thaliana, increasing or generating the activity of a gene product non-targeted with the activity of a “YFL0190-protein”, preferably being encoded by a gene comprising the nucleic acid sequence SEQ ID NO. 64144, preferably the coding region thereof, conferred the production of or the increase in myo-inositol compared with the wild type control.

The nucleic acid sequence of Yfl019c from Saccharomyces cerevisiae, e.g. as shown in the respective line in column 5 of Table I, application no. 17, has been published in A. And the activity of the gene product thereof is the activity of YFL019C-protein.

Accordingly, in one embodiment, the process of the present invention for producing sucrose in a non-human organism, like a microorganism or a plant or a part thereof, comprises increasing or generating the activity of a gene product with the activity of a gene product conferring the activity of “YFL0190-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said Yfl019c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Yfl019c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Yfl019c, or a functional equivalent or a homolog thereof as depicted in column 8 of Table II, application no. 17, preferably a homolog or functional equivalent as depicted in column 8 of Table II B, application no. 17, and being depicted in the same respective line as said Yfl019c, and preferably the activity is increased non-targeted, whereby the respective line disclose in column 7 the fine chemical sucrose.

Accordingly, in one embodiment, the molecule which activity is to be increased in the process of the invention is the gene product with an activity as a “YFL0190-protein”, preferably it is the molecule of section (a) or (b) of this paragraph.

In particular, it was observed that in plants, especially in Arabidopsis thaliana, increasing or generating the activity of a gene product non-targeted with the activity of a “YFL019C-protein”, preferably being encoded by a gene comprising the nucleic acid sequence SEQ ID NO. 64144, preferably the coding region thereof, conferred the production of or the increase in sucrose compared with the wild type control.

The nucleic acid sequence of Yfr053c from Saccharomyces cerevisiae, e.g. as shown in the respective line in column 5 of Table I, application no. 17, has been published in A. And the activity of the gene product thereof is the activity of hexokinase.

Accordingly, in one embodiment, the process of the present invention for producing sucrose in a non-human organism, like a microorganism or a plant or a part thereof, comprises increasing or generating the activity of a gene product with the activity of a gene product conferring the activity of “hexokinase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said Yfr053c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Yfr053c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Yfr053c, or a functional equivalent or a homolog thereof as depicted in column 8 of Table II, application no. 17, preferably a homolog or functional equivalent as depicted in column 8 of Table II B, application no. 17, and being depicted in the same respective line as said Yfr053c, and preferably the activity is increased non-targeted, whereby the respective line disclose in column 7 the fine chemical sucrose.

Accordingly, in one embodiment, the molecule which activity is to be increased in the process of the invention is the gene product with an activity as a “hexokinase”, preferably it is the molecule of section (a) or (b) of this paragraph.

In particular, it was observed that in plants, especially in Arabidopsis thaliana, increasing or generating the activity of a gene product non-targeted with the activity of a “hexokinase”, preferably being encoded by a gene comprising the nucleic acid sequence SEQ ID NO. 149219, preferably the coding region thereof, conferred the production of or the increase in sucrose compared with the wild type control.

The nucleic acid sequence of Ygl039w from Saccharomyces cerevisiae, e.g. as shown in the respective line in column 5 of Table I, application no. 17, has been published in A. And the activity of the gene product thereof is the activity of oxidoreductase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “oxidoreductase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Ygl039w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ygl039w, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ygl039w, or a functional equivalent or a homolog thereof as depicted in column 8 of Table II, application no. 17, preferably a homolog or functional equivalent as depicted in column 8 of Table II B, application no. 17, and being depicted in the same respective line as said Ygl039w, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product plastidic with the activity of a “oxidoreductase”, preferably being encoded by a gene comprising the nucleic acid sequence SEQ ID NO. 120436, preferably the coding region thereof, conferred the production of or the increase in glucose-1-phosphate compared with the wild type control.

The nucleic acid sequence of Ygl039w from Saccharomyces cerevisiae, e.g. as shown in the respective line in column 5 of Table I, application no. 17, has been published in A. And the activity of the gene product thereof is the activity of oxidoreductase.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “oxidoreductase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said Ygl039w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ygl039w, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ygl039w, or a functional equivalent or a homolog thereof as depicted in column 8 of Table II, application no. 17, preferably a homolog or functional equivalent as depicted in column 8 of Table II B, application no. 17, and being depicted in the same respective line as said Ygl039w, and preferably the activity is increased plastidic, whereby the respective line disclose in column 7 the fine chemical 3,4,5-trihydroxypentanoic acid.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product plastidic with the activity of a “oxidoreductase”, preferably being encoded by a gene comprising the nucleic acid sequence SEQ ID NO. 120436, preferably the coding region thereof, conferred the production of or the increase in 3,4,5-trihydroxypentanoic acid compared with the wild type control.

The nucleic acid sequence of Ygl096w from Saccharomyces cerevisiae, e.g. as shown in the respective line in column 5 of Table I, application no. 17, has been published in A. And the activity of the gene product thereof is the activity of ygl096w-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “ygl096w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Ygl096w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ygl096w, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ygl096w, or a functional equivalent or a homolog thereof as depicted in column 8 of Table II, application no. 17, preferably a homolog or functional equivalent as depicted in column 8 of Table II B, application no. 17, and being depicted in the same respective line as said Ygl096w, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “ygl096w-protein”, preferably it is the molecule of section (a) or (b) of this paragraph.

In particular, it was observed that in plants, especially in Arabidopsis thaliana, increasing or generating the activity of a gene product plastidic with the activity of a “ygl096w-protein”, preferably being encoded by a gene comprising the nucleic acid sequence SEQ ID NO. 90278, preferably the coding region thereof, conferred the production of or the increase in glucose-1-phosphate compared with the wild type control.

The nucleic acid sequence of Ygl096w from Saccharomyces cerevisiae, e.g. as shown in the respective line in column 5 of Table I, application no. 17, has been published in A. And the activity of the gene product thereof is the activity of ygl096w-protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “ygl096w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said Ygl096w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ygl096w, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ygl096w, or a functional equivalent or a homolog thereof as depicted in column 8 of Table II, application no. 17, preferably a homolog or functional equivalent as depicted in column 8 of Table II B, application no. 17, and being depicted in the same respective line as said Ygl096w, and preferably the activity is increased plastidic, whereby the respective line disclose in column 7 the fine chemical 3,4,5-trihydroxypentanoic acid.

Accordingly, in one embodiment, the molecule which activity is to be increased in the process of the invention is the gene product with an activity as a “ygl096w-protein”, preferably it is the molecule of section (a) or (b) of this paragraph.

In particular, it was observed that in plants, especially in Arabidopsis thaliana, increasing or generating the activity of a gene product plastidic with the activity of a “ygl096w-protein”, preferably being encoded by a gene comprising the nucleic acid sequence SEQ ID NO. 90278, preferably the coding region thereof, conferred the production of or the increase in 3,4,5-trihydroxypentanoic acid compared with the wild type control.

The nucleic acid sequence of Ygl237c from Saccharomyces cerevisiae, e.g. as shown in the respective line in column 5 of Table I, application no. 17, has been published in A. And the activity of the gene product thereof is the activity of ygl237c-protein.

Accordingly, in one embodiment, the process of the present invention for producing raffinose in a non-human organism, like a microorganism or a plant or a part thereof, comprises increasing or generating the activity of a gene product with the activity of a gene product conferring the activity of “ygl237c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said Ygl237c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ygl237c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ygl237c, or a functional equivalent or a homolog thereof as depicted in column 8 of Table II, application no. 17, preferably a homolog or functional equivalent as depicted in column 8 of Table II B, application no. 17, and being depicted in the same respective line as said Ygl237c, and preferably the activity is increased non-targeted, whereby the respective line disclose in column 7 the fine chemical raffinose.

Accordingly, in one embodiment, the molecule which activity is to be increased in the process of the invention is the gene product with an activity as a “ygl237c-protein”, preferably it is the molecule of section (a) or (b) of this paragraph.

In particular, it was observed that in plants, especially in Arabidopsis thaliana, increasing or generating the activity of a gene product non-targeted with the activity of a “ygl237c-protein”, preferably being encoded by a gene comprising the nucleic acid sequence SEQ ID NO. 64177, preferably the coding region thereof, conferred the production of or the increase in raffinose compared with the wild type control.

The nucleic acid sequence of Ygl256w from Saccharomyces cerevisiae, e.g. as shown in the respective line in column 5 of Table I, application no. 17, has been published in A. And the activity of the gene product thereof is the activity of alcohol dehydrogenase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “alcohol dehydrogenase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Ygl256w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ygl256w, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ygl256w, or a functional equivalent or a homolog thereof as depicted in column 8 of Table II, application no. 17, preferably a homolog or functional equivalent as depicted in column 8 of Table II B, application no. 17, and being depicted in the same respective line as said Ygl256w, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “alcohol dehydrogenase”, preferably it is the molecule of section (a) or (b) of this paragraph.

In particular, it was observed that in plants, especially in Arabidopsis thaliana, increasing or generating the activity of a gene product plastidic with the activity of a “alcohol dehydrogenase”, preferably being encoded by a gene comprising the nucleic acid sequence SEQ ID NO. 130742, preferably the coding region thereof, conferred the production of or the increase in glucose-1-phosphate compared with the wild type control.

The nucleic acid sequence of Ygl256w from Saccharomyces cerevisiae, e.g. as shown in the respective line in column 5 of Table I, application no. 17, has been published in A. And the activity of the gene product thereof is the activity of alcohol dehydrogenase.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “alcohol dehydrogenase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said Ygl256w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ygl256w, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ygl256w, or a functional equivalent or a homolog thereof as depicted in column 8 of Table II, application no. 17, preferably a homolog or functional equivalent as depicted in column 8 of Table II B, application no. 17, and being depicted in the same respective line as said Ygl256w, and preferably the activity is increased plastidic, whereby the respective line disclose in column 7 the fine chemical 3,4,5-trihydroxypentanoic acid.

Accordingly, in one embodiment, the molecule which activity is to be increased in the process of the invention is the gene product with an activity as a “alcohol dehydrogenase”, preferably it is the molecule of section (a) or (b) of this paragraph.

In particular, it was observed that in plants, especially in Arabidopsis thaliana, increasing or generating the activity of a gene product plastidic with the activity of a “alcohol dehydrogenase”, preferably being encoded by a gene comprising the nucleic acid sequence SEQ ID NO. 130742, preferably the coding region thereof, conferred the production of or the increase in 3,4,5-trihydroxypentanoic acid compared with the wild type control.

The nucleic acid sequence of Yhl013c from Saccharomyces cerevisiae, e.g. as shown in the respective line in column 5 of Table I, application no. 17, has been published in A. And the activity of the gene product thereof is the activity of yhl013c-protein.

Accordingly, in one embodiment, the process of the present invention for producing glucose in a non-human organism, like a microorganism or a plant or a part thereof, comprises increasing or generating the activity of a gene product with the activity of a gene product conferring the activity of “yhl013c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said Yhl013c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Yhl013c, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Yhl013c, or a functional equivalent or a homolog thereof as depicted in column 8 of Table II, application no. 17, preferably a homolog or functional equivalent as depicted in column 8 of Table II B, application no. 17, and being depicted in the same respective line as said Yhl013c, and preferably the activity is increased plastidic, whereby the respective line disclose in column 7 the fine chemical glucose.

Accordingly, in one embodiment, the molecule which activity is to be increased in the process of the invention is the gene product with an activity as a “yhl013c-protein”, preferably it is the molecule of section (a) or (b) of this paragraph.

In particular, it was observed that in plants, especially in Arabidopsis thaliana, increasing or generating the activity of a gene product plastidic with the activity of a “yhl013c-protein”, preferably being encoded by a gene comprising the nucleic acid sequence SEQ ID NO. 14715, preferably the coding region thereof, conferred the production of or the increase in glucose compared with the wild type control.

The nucleic acid sequence of Yhl013c from Saccharomyces cerevisiae, e.g. as shown in the respective line in column 5 of Table I, application no. 17, has been published in A. And the activity of the gene product thereof is the activity of yhl013c-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “yhl013c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Yhl013c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Yhl013c, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Yhl013c, or a functional equivalent or a homolog thereof as depicted in column 8 of Table II, application no. 17, preferably a homolog or functional equivalent as depicted in column 8 of Table II B, application no. 17, and being depicted in the same respective line as said Yhl013c, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “yhl013c-protein”, preferably it is the molecule of section (a) or (b) of this paragraph.

In particular, it was observed that in plants, especially in Arabidopsis thaliana, increasing or generating the activity of a gene product plastidic with the activity of a “yhl013c-protein”, preferably being encoded by a gene comprising the nucleic acid sequence SEQ ID NO. 14715, preferably the coding region thereof, conferred the production of or the increase in glucose-1-phosphate compared with the wild type control.

The nucleic acid sequence of Yil074c from Saccharomyces cerevisiae, e.g. as shown in the respective line in column 5 of Table I, application no. 17, has been published in A. And the activity of the gene product thereof is the activity of 3-phosphoglycerate dehydrogenase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “3-phosphoglycerate dehydrogenase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Yil074c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Yil074c, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Yil074c, or a functional equivalent or a homolog thereof as depicted in column 8 of Table II, application no. 17, preferably a homolog or functional equivalent as depicted in column 8 of Table II B, application no. 17, and being depicted in the same respective line as said Yil074c, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “3-phosphoglycerate dehydrogenase”, preferably it is the molecule of section (a) or (b) of this paragraph.

In particular, it was observed that in plants, especially in Arabidopsis thaliana, increasing or generating the activity of a gene product plastidic with the activity of a “3-phosphoglycerate dehydrogenase”, preferably being encoded by a gene comprising the nucleic acid sequence SEQ ID NO. 64563, preferably the coding region thereof, conferred the production of or the increase in glucose-1-phosphate compared with the wild type control.

The nucleic acid sequence of Yil083c from Saccharomyces cerevisiae, e.g. as shown in the respective line in column 5 of Table I, application no. 17, has been published in A. And the activity of the gene product thereof is the activity of YIL083C-protein.

Accordingly, in one embodiment, the process of the present invention for producing fructose in a non-human organism, like a microorganism or a plant or a part thereof, comprises increasing or generating the activity of a gene product with the activity of a gene product conferring the activity of “YIL083C-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said Yil083c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Yil083c, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Yil083c, or a functional equivalent or a homolog thereof as depicted in column 8 of Table II, application no. 17, preferably a homolog or functional equivalent as depicted in column 8 of Table II B, application no. 17, and being depicted in the same respective line as said Yil083c, and preferably the activity is increased plastidic, whereby the respective line disclose in column 7 the fine chemical fructose.

Accordingly, in one embodiment, the molecule which activity is to be increased in the process of the invention is the gene product with an activity as a “YIL083C-protein”, preferably it is the molecule of section (a) or (b) of this paragraph.

In particular, it was observed that in plants, especially in Arabidopsis thaliana, increasing or generating the activity of a gene product plastidic with the activity of a “YIL083C-protein”, preferably being encoded by a gene comprising the nucleic acid sequence SEQ ID NO. 149454, preferably the coding region thereof, conferred the production of or the increase in fructose compared with the wild type control.

The nucleic acid sequence of Yjl073w from Saccharomyces cerevisiae, e.g. as shown in the respective line in column 5 of Table I, application no. 17, has been published in A. And the activity of the gene product thereof is the activity of DnaJ-like chaperone.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “DnaJ-like chaperone”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said Yjl073w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Yjl073w, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Yjl073w, or a functional equivalent or a homolog thereof as depicted in column 8 of Table II, application no. 17, preferably a homolog or functional equivalent as depicted in column 8 of Table II B, application no. 17, and being depicted in the same respective line as said Yjl073w, and preferably the activity is increased plastidic, whereby the respective line disclose in column 7 the fine chemical myo-inositol.

Accordingly, in one embodiment, the molecule which activity is to be increased in the process of the invention is the gene product with an activity as a “DnaJ-like chaperone”, preferably it is the molecule of section (a) or (b) of this paragraph.

In particular, it was observed that in plants, especially in Arabidopsis thaliana, increasing or generating the activity of a gene product plastidic with the activity of a “DnaJ-like chaperone”, preferably being encoded by a gene comprising the nucleic acid sequence SEQ ID NO. 64964, preferably the coding region thereof, conferred the production of or the increase in myo-inositol compared with the wild type control.

The nucleic acid sequence of Yjl127w-a from Saccharomyces cerevisiae, e.g. as shown in the respective line in column 5 of Table I, application no. 17, has been published in A. And the activity of the gene product thereof is the activity of YJL127W-A-protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “YJL127W-A-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said Yjl127w-a, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Yjl127w-a, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Yjl127w-a, or a functional equivalent or a homolog thereof as depicted in column 8 of Table II, application no. 17, preferably a homolog or functional equivalent as depicted in column 8 of Table II B, application no. 17, and being depicted in the same respective line as said Yjl127w-a, and preferably the activity is increased non-targeted, whereby the respective line disclose in column 7 the fine chemical 3,4,5-trihydroxypentanoic acid.

Accordingly, in one embodiment, the molecule which activity is to be increased in the process of the invention is the gene product with an activity as a “YJL127W-A-protein”, preferably it is the molecule of section (a) or (b) of this paragraph.

In particular, it was observed that in plants, especially in Arabidopsis thaliana, increasing or generating the activity of a gene product non-targeted with the activity of a “YJL127W-A-protein”, preferably being encoded by a gene comprising the nucleic acid sequence SEQ ID NO. 97631, preferably the coding region thereof, conferred the production of or the increase in 3,4,5-trihydroxypentanoic acid compared with the wild type control.

The nucleic acid sequence of Yjl153c from Saccharomyces cerevisiae, e.g. as shown in the respective line in column 5 of Table I, application no. 17, has been published in A. And the activity of the gene product thereof is the activity of inositol-3-phosphate synthase.

Accordingly, in one embodiment, the process of the present invention for producing fructose in a non-human organism, like a microorganism or a plant or a part thereof, comprises increasing or generating the activity of a gene product with the activity of a gene product conferring the activity of “inositol-3-phosphate synthase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said Yjl153c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Yjl153c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Yjl153c, or a functional equivalent or a homolog thereof as depicted in column 8 of Table II, application no. 17, preferably a homolog or functional equivalent as depicted in column 8 of Table II B, application no. 17, and being depicted in the same respective line as said Yjl153c, and preferably the activity is increased non-targeted, whereby the respective line disclose in column 7 the fine chemical fructose.

Accordingly, in one embodiment, the molecule which activity is to be increased in the process of the invention is the gene product with an activity as a “inositol-3-phosphate synthase”, preferably it is the molecule of section (a) or (b) of this paragraph.

In particular, it was observed that in plants, especially in Arabidopsis thaliana, increasing or generating the activity of a gene product non-targeted with the activity of a “inositol-3-phosphate synthase”, preferably being encoded by a gene comprising the nucleic acid sequence SEQ ID NO. 149632, preferably the coding region thereof, conferred the production of or the increase in fructose compared with the wild type control.

The nucleic acid sequence of Yjl153c from Saccharomyces cerevisiae, e.g. as shown in the respective line in column 5 of Table I, application no. 17, has been published in A. And the activity of the gene product thereof is the activity of inositol-3-phosphate synthase.

Accordingly, in one embodiment, the process of the present invention for producing galactitol in a non-human organism, like a microorganism or a plant or a part thereof, comprises increasing or generating the activity of a gene product with the activity of a gene product conferring the activity of “inositol-3-phosphate synthase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical galactitol), application no. 17, preferably the coding region thereof, or a homolog or a fragment thereof, and being depicted in the same respective line as said Yjl153c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Yjl153c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Yjl153c, or a functional equivalent or a homolog thereof as depicted in column 8 of Table II, application no. 17, preferably a homolog or functional equivalent as depicted in column 8 of Table II B, application no. 17, and being depicted in the same respective line as said Yjl153c, and preferably the activity is increased non-targeted, whereby the respective line disclose in column 7 the fine chemical galactitol.

Accordingly, in one embodiment, the molecule which activity is to be increased in the process of the invention is the gene product with an activity as a “inositol-3-phosphate synthase”, preferably it is the molecule of section (a) or (b) of this paragraph.

In particular, it was observed that in plants, especially in Arabidopsis thaliana, increasing or generating the activity of a gene product non-targeted with the activity of a “inositol-3-phosphate synthase”, preferably being encoded by a gene comprising the nucleic acid sequence SEQ ID NO. 149632, preferably the coding region thereof, conferred the production of or the increase in galactitol compared with the wild type control.

The nucleic acid sequence of Yjl153c from Saccharomyces cerevisiae, e.g. as shown in the respective line in column 5 of Table I, application no. 17, has been published in A. And the activity of the gene product thereof is the activity of inositol-3-phosphate synthase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “inositol-3-phosphate synthase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said Yjl153c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Yjl153c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Yjl153c, or a functional equivalent or a homolog thereof as depicted in column 8 of Table II, application no. 17, preferably a homolog or functional equivalent as depicted in column 8 of Table II B, application no. 17, and being depicted in the same respective line as said Yjl153c, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “inositol-3-phosphate synthase”, preferably it is the molecule of section (a) or (b) of this paragraph.

In particular, it was observed that in plants, especially in Arabidopsis thaliana, increasing or generating the activity of a gene product non-targeted with the activity of a “inositol-3-phosphate synthase”, preferably being encoded by a gene comprising the nucleic acid sequence SEQ ID NO. 149632, preferably the coding region thereof, conferred the production of or the increase in myo-inositol compared with the wild type control.

The nucleic acid sequence of Yjl219w from Saccharomyces cerevisiae, e.g. as shown in the respective line in column 5 of Table I, application no. 17, has been published in A. And the activity of the gene product thereof is the activity of hexose transporter.

Accordingly, in one embodiment, the process of the present invention for producing sorbitol in a non-human organism, like a microorganism or a plant or a part thereof, comprises increasing or generating the activity of a gene product with the activity of a gene product conferring the activity of “hexose transporter”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical sorbitol), application no. 17, preferably the coding region thereof, or a homolog or a fragment thereof, and being depicted in the same respective line as said Yjl219w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Yjl219w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Yjl219w, or a functional equivalent or a homolog thereof as depicted in column 8 of Table II, application no. 17, preferably a homolog or functional equivalent as depicted in column 8 of Table II B, application no. 17, and being depicted in the same respective line as said Yjl219w, and preferably the activity is increased non-targeted, whereby the respective line disclose in column 7 the fine chemical sorbitol.

Accordingly, in one embodiment, the molecule which activity is to be increased in the process of the invention is the gene product with an activity as a “hexose transporter”, preferably it is the molecule of section (a) or (b) of this paragraph.

In particular, it was observed that in plants, especially in Arabidopsis thaliana, increasing or generating the activity of a gene product non-targeted with the activity of a “hexose transporter”, preferably being encoded by a gene comprising the nucleic acid sequence SEQ ID NO. 97635, preferably the coding region thereof, conferred the production of or the increase in sorbitol compared with the wild type control.

The nucleic acid sequence of Yjr066w from Saccharomyces cerevisiae, e.g. as shown in the respective line in column 5 of Table I, application no. 17, has been published in A. And the activity of the gene product thereof is the activity of protein kinase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “protein kinase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said Yjr066w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Yjr066w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Yjr066w, or a functional equivalent or a homolog thereof as depicted in column 8 of Table II, application no. 17, preferably a homolog or functional equivalent as depicted in column 8 of Table II B, application no. 17, and being depicted in the same respective line as said Yjr066w, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 149801, preferably the coding region thereof, conferred the production of or the increase in myo-inositol compared with the wild type control.

The nucleic acid sequence of Yjr130c from Saccharomyces cerevisiae, e.g. as shown in the respective line in column 5 of Table I, application no. 17, has been published in A. And the activity of the gene product thereof is the activity of cystathionine gamma-synthase.

Accordingly, in one embodiment, the process of the present invention for producing glucose in a non-human organism, like a microorganism or a plant or a part thereof, comprises increasing or generating the activity of a gene product with the activity of a gene product conferring the activity of “cystathionine gamma-synthase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said Yjr130c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Yjr130c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Yjr130c, or a functional equivalent or a homolog thereof as depicted in column 8 of Table II, application no. 17, preferably a homolog or functional equivalent as depicted in column 8 of Table II B, application no. 17, and being depicted in the same respective line as said Yjr130c, and preferably the activity is increased non-targeted, whereby the respective line disclose in column 7 the fine chemical glucose.

Accordingly, in one embodiment, the molecule which activity is to be increased in the process of the invention is the gene product with an activity as a “cystathionine gamma-synthase”, preferably it is the molecule of section (a) or (b) of this paragraph.

In particular, it was observed that in plants, especially in Arabidopsis thaliana, increasing or generating the activity of a gene product non-targeted with the activity of a “cystathionine gamma-synthase”, preferably being encoded by a gene comprising the nucleic acid sequence SEQ ID NO. 14843, preferably the coding region thereof, conferred the production of or the increase in glucose compared with the wild type control.

The nucleic acid sequence of Ykl060c from Saccharomyces cerevisiae, e.g. as shown in the respective line in column 5 of Table I, application no. 17, has been published in A. And the activity of the gene product thereof is the activity of fructose biphosphate aldolase.

Accordingly, in one embodiment, the process of the present invention for producing fructose in a non-human organism, like a microorganism or a plant or a part thereof, comprises increasing or generating the activity of a gene product with the activity of a gene product conferring the activity of “fructose biphosphate aldolase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said Ykl060c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ykl060c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ykl060c, or a functional equivalent or a homolog thereof as depicted in column 8 of Table II, application no. 17, preferably a homolog or functional equivalent as depicted in column 8 of Table II B, application no. 17, and being depicted in the same respective line as said Ykl060c, and preferably the activity is increased non-targeted, whereby the respective line disclose in column 7 the fine chemical fructose.

Accordingly, in one embodiment, the molecule which activity is to be increased in the process of the invention is the gene product with an activity as a “fructose biphosphate aldolase”, preferably it is the molecule of section (a) or (b) of this paragraph.

In particular, it was observed that in plants, especially in Arabidopsis thaliana, increasing or generating the activity of a gene product non-targeted with the activity of a “fructose biphosphate aldolase”, preferably being encoded by a gene comprising the nucleic acid sequence SEQ ID NO. 149868, preferably the coding region thereof, conferred the production of or the increase in fructose compared with the wild type control.

The nucleic acid sequence of Ykl060c from Saccharomyces cerevisiae, e.g. as shown in the respective line in column 5 of Table I, application no. 17, has been published in A. And the activity of the gene product thereof is the activity of fructose biphosphate aldolase.

Accordingly, in one embodiment, the process of the present invention for producing sedoheptulose-7-phosphate in a non-human organism, like a microorganism or a plant or a part thereof, comprises increasing or generating the activity of a gene product with the activity of a gene product conferring the activity of “fructose biphosphate aldolase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical sedoheptulose-7-phosphate), application no. 17, preferably the coding region thereof, or a homolog or a fragment thereof, and being depicted in the same respective line as said Ykl060c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ykl060c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ykl060c, or a functional equivalent or a homolog thereof as depicted in column 8 of Table II, application no. 17, preferably a homolog or functional equivalent as depicted in column 8 of Table II B, application no. 17, and being depicted in the same respective line as said Ykl060c, and preferably the activity is increased non-targeted, whereby the respective line disclose in column 7 the fine chemical sedoheptulose-7-phosphate.

Accordingly, in one embodiment, the molecule which activity is to be increased in the process of the invention is the gene product with an activity as a “fructose biphosphate aldolase”, preferably it is the molecule of section (a) or (b) of this paragraph.

In particular, it was observed that in plants, especially in Arabidopsis thaliana, increasing or generating the activity of a gene product non-targeted with the activity of a “fructose biphosphate aldolase”, preferably being encoded by a gene comprising the nucleic acid sequence SEQ ID NO. 149868, preferably the coding region thereof, conferred the production of or the increase in sedoheptulose-7-phosphate compared with the wild type control.

The nucleic acid sequence of Ykl192c from Saccharomyces cerevisiae, e.g. as shown in the respective line in column 5 of Table I, application no. 17, has been published in A. And the activity of the gene product thereof is the activity of acyl carrier protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “acyl carrier protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Ykl192c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ykl192c, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ykl192c, or a functional equivalent or a homolog thereof as depicted in column 8 of Table II, application no. 17, preferably a homolog or functional equivalent as depicted in column 8 of Table II B, application no. 17, and being depicted in the same respective line as said Ykl192c, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “acyl carrier protein”, preferably it is the molecule of section (a) or (b) of this paragraph.

In particular, it was observed that in plants, especially in Arabidopsis thaliana, increasing or generating the activity of a gene product plastidic with the activity of a “acyl carrier protein”, preferably being encoded by a gene comprising the nucleic acid sequence SEQ ID NO. 150063, preferably the coding region thereof, conferred the production of or the increase in glucose-1-phosphate compared with the wild type control.

The nucleic acid sequence of Ykr072c from Saccharomyces cerevisiae, e.g. as shown in the respective line in column 5 of Table I, application no. 17, has been published in A. And the activity of the gene product thereof is the activity of halotolerance protein.

Accordingly, in one embodiment, the process of the present invention for producing sucrose in a non-human organism, like a microorganism or a plant or a part thereof, comprises increasing or generating the activity of a gene product with the activity of a gene product conferring the activity of “halotolerance protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said Ykr072c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ykr072c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ykr072c, or a functional equivalent or a homolog thereof as depicted in column 8 of Table II, application no. 17, preferably a homolog or functional equivalent as depicted in column 8 of Table II B, application no. 17, and being depicted in the same respective line as said Ykr072c, and preferably the activity is increased non-targeted, whereby the respective line disclose in column 7 the fine chemical sucrose.

Accordingly, in one embodiment, the molecule which activity is to be increased in the process of the invention is the gene product with an activity as a “halotolerance protein”, preferably it is the molecule of section (a) or (b) of this paragraph.

In particular, it was observed that in plants, especially in Arabidopsis thaliana, increasing or generating the activity of a gene product non-targeted with the activity of a “halotolerance protein”, preferably being encoded by a gene comprising the nucleic acid sequence SEQ ID NO. 130903, preferably the coding region thereof, conferred the production of or the increase in sucrose compared with the wild type control.

The nucleic acid sequence of Ykr097w from Saccharomyces cerevisiae, e.g. as shown in the respective line in column 5 of Table I, application no. 17, has been published in A. And the activity of the gene product thereof is the activity of phosphoenolpyruvate carboxykinase.

Accordingly, in one embodiment, the process of the present invention for producing glucose in a non-human organism, like a microorganism or a plant or a part thereof, comprises increasing or generating the activity of a gene product with the activity of a gene product conferring the activity of “phosphoenolpyruvate carboxykinase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said Ykr097w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ykr097w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ykr097w, or a functional equivalent or a homolog thereof as depicted in column 8 of Table II, application no. 17, preferably a homolog or functional equivalent as depicted in column 8 of Table II B, application no. 17, and being depicted in the same respective line as said Ykr097w, and preferably the activity is increased non-targeted, whereby the respective line disclose in column 7 the fine chemical glucose.

Accordingly, in one embodiment, the molecule which activity is to be increased in the process of the invention is the gene product with an activity as a “phosphoenolpyruvate carboxykinase”, preferably it is the molecule of section (a) or (b) of this paragraph.

In particular, it was observed that in plants, especially in Arabidopsis thaliana, increasing or generating the activity of a gene product non-targeted with the activity of a “phosphoenolpyruvate carboxykinase”, preferably being encoded by a gene comprising the nucleic acid sequence SEQ ID NO. 98075, preferably the coding region thereof, conferred the production of or the increase in glucose compared with the wild type control.

The nucleic acid sequence of Ykr097w from Saccharomyces cerevisiae, e.g. as shown in the respective line in column 5 of Table I, application no. 17, has been published in A. And the activity of the gene product thereof is the activity of phosphoenolpyruvate carboxykinase.

Accordingly, in one embodiment, the process of the present invention for producing glucose in a non-human organism, like a microorganism or a plant or a part thereof, comprises increasing or generating the activity of a gene product with the activity of a gene product conferring the activity of “phosphoenolpyruvate carboxykinase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said Ykr097w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ykr097w, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ykr097w, or a functional equivalent or a homolog thereof as depicted in column 8 of Table II, application no. 17, preferably a homolog or functional equivalent as depicted in column 8 of Table II B, application no. 17, and being depicted in the same respective line as said Ykr097w, and preferably the activity is increased plastidic, whereby the respective line disclose in column 7 the fine chemical glucose.

Accordingly, in one embodiment, the molecule which activity is to be increased in the process of the invention is the gene product with an activity as a “phosphoenolpyruvate carboxykinase”, preferably it is the molecule of section (a) or (b) of this paragraph.

In particular, it was observed that in plants, especially in Arabidopsis thaliana, increasing or generating the activity of a gene product plastidic with the activity of a “phosphoenolpyruvate carboxykinase”, preferably being encoded by a gene comprising the nucleic acid sequence SEQ ID NO. 98075, preferably the coding region thereof, conferred the production of or the increase in glucose compared with the wild type control.

The nucleic acid sequence of Ylr025w from Saccharomyces cerevisiae, e.g. as shown in the respective line in column 5 of Table I, application no. 17, has been published in A. And the activity of the gene product thereof is the activity of vacuolar sorting protein.

Accordingly, in one embodiment, the process of the present invention for producing sucrose in a non-human organism, like a microorganism or a plant or a part thereof, comprises increasing or generating the activity of a gene product with the activity of a gene product conferring the activity of “vacuolar sorting protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said Ylr025w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ylr025w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ylr025w, or a functional equivalent or a homolog thereof as depicted in column 8 of Table II, application no. 17, preferably a homolog or functional equivalent as depicted in column 8 of Table II B, application no. 17, and being depicted in the same respective line as said Ylr025w, and preferably the activity is increased non-targeted, whereby the respective line disclose in column 7 the fine chemical sucrose.

Accordingly, in one embodiment, the molecule which activity is to be increased in the process of the invention is the gene product with an activity as a “vacuolar sorting protein”, preferably it is the molecule of section (a) or (b) of this paragraph.

In particular, it was observed that in plants, especially in Arabidopsis thaliana, increasing or generating the activity of a gene product non-targeted with the activity of a “vacuolar sorting protein”, preferably being encoded by a gene comprising the nucleic acid sequence SEQ ID NO. 150603, preferably the coding region thereof, conferred the production of or the increase in sucrose compared with the wild type control.

The nucleic acid sequence of Ylr043c from Saccharomyces cerevisiae, e.g. as shown in the respective line in column 5 of Table I, application no. 17, has been published in A. And the activity of the gene product thereof is the activity of thioredoxin.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “thioredoxin”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Ylr043c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ylr043c, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ylr043c, or a functional equivalent or a homolog thereof as depicted in column 8 of Table II, application no. 17, preferably a homolog or functional equivalent as depicted in column 8 of Table II B, application no. 17, and being depicted in the same respective line as said Ylr043c, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “thioredoxin”, preferably it is the molecule of section (a) or (b) of this paragraph.

In particular, it was observed that in plants, especially in Arabidopsis thaliana, increasing or generating the activity of a gene product plastidic with the activity of a “thioredoxin”, preferably being encoded by a gene comprising the nucleic acid sequence SEQ ID NO. 91045, preferably the coding region thereof, conferred the production of or the increase in glucose-1-phosphate compared with the wild type control.

The nucleic acid sequence of Ylr081w from Saccharomyces cerevisiae, e.g. as shown in the respective line in column 5 of Table I, application no. 17, has been published in A. And the activity of the gene product thereof is the activity of hexose transporter.

Accordingly, in one embodiment, the process of the present invention for producing fructose in a non-human organism, like a microorganism or a plant or a part thereof, comprises increasing or generating the activity of a gene product with the activity of a gene product conferring the activity of “hexose transporter”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said Ylr081w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ylr081w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ylr081w, or a functional equivalent or a homolog thereof as depicted in column 8 of Table II, application no. 17, preferably a homolog or functional equivalent as depicted in column 8 of Table II B, application no. 17, and being depicted in the same respective line as said Ylr081w, and preferably the activity is increased non-targeted, whereby the respective line disclose in column 7 the fine chemical fructose.

Accordingly, in one embodiment, the molecule which activity is to be increased in the process of the invention is the gene product with an activity as a “hexose transporter”, preferably it is the molecule of section (a) or (b) of this paragraph.

In particular, it was observed that in plants, especially in Arabidopsis thaliana, increasing or generating the activity of a gene product non-targeted with the activity of a “hexose transporter”, preferably being encoded by a gene comprising the nucleic acid sequence SEQ ID NO. 150682, preferably the coding region thereof, conferred the production of or the increase in fructose compared with the wild type control.

The nucleic acid sequence of Ymr120c from Saccharomyces cerevisiae, e.g. as shown in the respective line in column 5 of Table I, application no. 17, has been published in A. And the activity of the gene product thereof is the activity of purine biosynthesis protein.

Accordingly, in one embodiment, the process of the present invention for producing verbascose in a non-human organism, like a microorganism or a plant or a part thereof, comprises increasing or generating the activity of a gene product with the activity of a gene product conferring the activity of “purine biosynthesis protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical verbascose), application no. 17, preferably the coding region thereof, or a homolog or a fragment thereof, and being depicted in the same respective line as said Ymr120c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ymr120c, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ymr120c, or a functional equivalent or a homolog thereof as depicted in column 8 of Table II, application no. 17, preferably a homolog or functional equivalent as depicted in column 8 of Table II B, application no. 17, and being depicted in the same respective line as said Ymr120c, and preferably the activity is increased plastidic, whereby the respective line disclose in column 7 the fine chemical verbascose.

Accordingly, in one embodiment, the molecule which activity is to be increased in the process of the invention is the gene product with an activity as a “purine biosynthesis protein”, preferably it is the molecule of section (a) or (b) of this paragraph.

In particular, it was observed that in plants, especially in Arabidopsis thaliana, increasing or generating the activity of a gene product plastidic with the activity of a “purine biosynthesis protein”, preferably being encoded by a gene comprising the nucleic acid sequence SEQ ID NO. 91973, preferably the coding region thereof, conferred the production of or the increase in verbascose compared with the wild type control.

The nucleic acid sequence of Ymr324c from Saccharomyces cerevisiae, e.g. as shown in the respective line in column 5 of Table I, application no. 17, has been published in A. And the activity of the gene product thereof is the activity of ymr324c-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “ymr324c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Ymr324c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ymr324c, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ymr324c, or a functional equivalent or a homolog thereof as depicted in column 8 of Table II, application no. 17, preferably a homolog or functional equivalent as depicted in column 8 of Table II B, application no. 17, and being depicted in the same respective line as said Ymr324c, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “ymr324c-protein”, preferably it is the molecule of section (a) or (b) of this paragraph.

In particular, it was observed that in plants, especially in Arabidopsis thaliana, increasing or generating the activity of a gene product plastidic with the activity of a “ymr324c-protein”, preferably being encoded by a gene comprising the nucleic acid sequence SEQ ID NO. 130986, preferably the coding region thereof, conferred the production of or the increase in glucose-1-phosphate compared with the wild type control.

The nucleic acid sequence of Ynl064c from Saccharomyces cerevisiae, e.g. as shown in the respective line in column 5 of Table I, application no. 17, has been published in A. And the activity of the gene product thereof is the activity of molecular chaperone.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “molecular chaperone”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said Ynl064c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ynl064c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ynl064c, or a functional equivalent or a homolog thereof as depicted in column 8 of Table II, application no. 17, preferably a homolog or functional equivalent as depicted in column 8 of Table II B, application no. 17, and being depicted in the same respective line as said Ynl064c, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “molecular chaperone”, preferably it is the molecule of section (a) or (b) of this paragraph.

In particular, it was observed that in plants, especially in Arabidopsis thaliana, increasing or generating the activity of a gene product non-targeted with the activity of a “molecular chaperone”, preferably being encoded by a gene comprising the nucleic acid sequence SEQ ID NO. 117495, preferably the coding region thereof, conferred the production of or the increase in myo-inositol compared with the wild type control.

The nucleic acid sequence of Ynl064c from Saccharomyces cerevisiae, e.g. as shown in the respective line in column 5 of Table I, application no. 17, has been published in A. And the activity of the gene product thereof is the activity of molecular chaperone.

Accordingly, in one embodiment, the process of the present invention for producing sucrose in a non-human organism, like a microorganism or a plant or a part thereof, comprises increasing or generating the activity of a gene product with the activity of a gene product conferring the activity of “molecular chaperone”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said Ynl064c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ynl064c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ynl064c, or a functional equivalent or a homolog thereof as depicted in column 8 of Table II, application no. 17, preferably a homolog or functional equivalent as depicted in column 8 of Table II B, application no. 17, and being depicted in the same respective line as said Ynl064c, and preferably the activity is increased non-targeted, whereby the respective line disclose in column 7 the fine chemical sucrose.

Accordingly, in one embodiment, the molecule which activity is to be increased in the process of the invention is the gene product with an activity as a “molecular chaperone”, preferably it is the molecule of section (a) or (b) of this paragraph.

In particular, it was observed that in plants, especially in Arabidopsis thaliana, increasing or generating the activity of a gene product non-targeted with the activity of a “molecular chaperone”, preferably being encoded by a gene comprising the nucleic acid sequence SEQ ID NO. 117495, preferably the coding region thereof, conferred the production of or the increase in sucrose compared with the wild type control.

The nucleic acid sequence of Ynr019w from Saccharomyces cerevisiae, e.g. as shown in the respective line in column 5 of Table I, application no. 17, has been published in A. And the activity of the gene product thereof is the activity of sterol O-acyltransferase.

Accordingly, in one embodiment, the process of the present invention for producing raffinose in a non-human organism, like a microorganism or a plant or a part thereof, comprises increasing or generating the activity of a gene product with the activity of a gene product conferring the activity of “sterol O-acyltransferase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said Ynr019w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ynr019w, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ynr019w, or a functional equivalent or a homolog thereof as depicted in column 8 of Table II, application no. 17, preferably a homolog or functional equivalent as depicted in column 8 of Table II B, application no. 17, and being depicted in the same respective line as said Ynr019w, and preferably the activity is increased plastidic, whereby the respective line disclose in column 7 the fine chemical raffinose.

Accordingly, in one embodiment, the molecule which activity is to be increased in the process of the invention is the gene product with an activity as a “sterol O-acyltransferase”, preferably it is the molecule of section (a) or (b) of this paragraph.

In particular, it was observed that in plants, especially in Arabidopsis thaliana, increasing or generating the activity of a gene product plastidic with the activity of a “sterol O-acyltransferase”, preferably being encoded by a gene comprising the nucleic acid sequence SEQ ID NO. 67646, preferably the coding region thereof, conferred the production of or the increase in raffinose compared with the wild type control.

The nucleic acid sequence of Yol160w from Saccharomyces cerevisiae, e.g. as shown in the respective line in column 5 of Table I, application no. 17, has been published in A. And the activity of the gene product thereof is the activity of yol160w-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “yol160w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said Yol160w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Yol160w, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Yol160w, or a functional equivalent or a homolog thereof as depicted in column 8 of Table II, application no. 17, preferably a homolog or functional equivalent as depicted in column 8 of Table II B, application no. 17, and being depicted in the same respective line as said Yol160w, and preferably the activity is increased plastidic, whereby the respective line disclose in column 7 the fine chemical myo-inositol.

Accordingly, in one embodiment, the molecule which activity is to be increased in the process of the invention is the gene product with an activity as a “yol160w-protein”, preferably it is the molecule of section (a) or (b) of this paragraph.

In particular, it was observed that in plants, especially in Arabidopsis thaliana, increasing or generating the activity of a gene product plastidic with the activity of a “yol160w-protein”, preferably being encoded by a gene comprising the nucleic acid sequence SEQ ID NO. 15179, preferably the coding region thereof, conferred the production of or the increase in myo-inositol compared with the wild type control.

The nucleic acid sequence of Yor163w from Saccharomyces cerevisiae, e.g. as shown in the respective line in column 5 of Table I, application no. 17, has been published in A. And the activity of the gene product thereof is the activity of phosphohydrolase.

Accordingly, in one embodiment, the process of the present invention for producing glucose in a non-human organism, like a microorganism or a plant or a part thereof, comprises increasing or generating the activity of a gene product with the activity of a gene product conferring the activity of “phosphohydrolase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said Yor163w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Yor163w, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Yor163w, or a functional equivalent or a homolog thereof as depicted in column 8 of Table II, application no. 17, preferably a homolog or functional equivalent as depicted in column 8 of Table II B, application no. 17, and being depicted in the same respective line as said Yor163w, and preferably the activity is increased plastidic, whereby the respective line disclose in column 7 the fine chemical glucose.

Accordingly, in one embodiment, the molecule which activity is to be increased in the process of the invention is the gene product with an activity as a “phosphohydrolase”, preferably it is the molecule of section (a) or (b) of this paragraph.

In particular, it was observed that in plants, especially in Arabidopsis thaliana, increasing or generating the activity of a gene product plastidic with the activity of a “phosphohydrolase”, preferably being encoded by a gene comprising the nucleic acid sequence SEQ ID NO. 151862, preferably the coding region thereof, conferred the production of or the increase in glucose compared with the wild type control.

The nucleic acid sequence of Yor244w from Saccharomyces cerevisiae, e.g. as shown in the respective line in column 5 of Table I, application no. 17, has been published in A. And the activity of the gene product thereof is the activity of histone acetyltransferase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “histone acetyltransferase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Yor244w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Yor244w, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Yor244w, or a functional equivalent or a homolog thereof as depicted in column 8 of Table II, application no. 17, preferably a homolog or functional equivalent as depicted in column 8 of Table II B, application no. 17, and being depicted in the same respective line as said Yor244w, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “histone acetyltransferase”, preferably it is the molecule of section (a) or (b) of this paragraph.

In particular, it was observed that in plants, especially in Arabidopsis thaliana, increasing or generating the activity of a gene product plastidic with the activity of a “histone acetyltransferase”, preferably being encoded by a gene comprising the nucleic acid sequence SEQ ID NO. 92540, preferably the coding region thereof, conferred the production of or the increase in glucose-1-phosphate compared with the wild type control.

The nucleic acid sequence of Ypl138c from Saccharomyces cerevisiae, e.g. as shown in the respective line in column 5 of Table I, application no. 17, has been published in A. And the activity of the gene product thereof is the activity of subunit of COMPASS-protein complex.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “subunit of COMPASS-protein complex”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said Ypl138c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ypl138c, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ypl138c, or a functional equivalent or a homolog thereof as depicted in column 8 of Table II, application no. 17, preferably a homolog or functional equivalent as depicted in column 8 of Table II B, application no. 17, and being depicted in the same respective line as said Ypl138c, and preferably the activity is increased plastidic, whereby the respective line disclose in column 7 the fine chemical 3,4,5-trihydroxypentanoic acid.

Accordingly, in one embodiment, the molecule which activity is to be increased in the process of the invention is the gene product with an activity as a “subunit of COMPASS-protein complex”, preferably it is the molecule of section (a) or (b) of this paragraph.

In particular, it was observed that in plants, especially in Arabidopsis thaliana, increasing or generating the activity of a gene product plastidic with the activity of a “subunit of COMPASSprotein complex”, preferably being encoded by a gene comprising the nucleic acid sequence SEQ ID NO. 151910, preferably the coding region thereof, conferred the production of or the increase in 3,4,5-trihydroxypentanoic acid compared with the wild type control.

The nucleic acid sequence of Ypl144w from Saccharomyces cerevisiae, e.g. as shown in the respective line in column 5 of Table I, application no. 17, has been published in A. And the activity of the gene product thereof is the activity of ypl144w-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “ypl144w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Ypl144w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ypl144w, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ypl144w, or a functional equivalent or a homolog thereof as depicted in column 8 of Table II, application no. 17, preferably a homolog or functional equivalent as depicted in column 8 of Table II B, application no. 17, and being depicted in the same respective line as said Ypl144w, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “ypl144w-protein”, preferably it is the molecule of section (a) or (b) of this paragraph.

In particular, it was observed that in plants, especially in Arabidopsis thaliana, increasing or generating the activity of a gene product plastidic with the activity of a “ypl144w-protein”, preferably being encoded by a gene comprising the nucleic acid sequence SEQ ID NO. 151927, preferably the coding region thereof, conferred the production of or the increase in glucose-1-phosphate compared with the wild type control.

The nucleic acid sequence of Ypl144w from Saccharomyces cerevisiae, e.g. as shown in the respective line in column 5 of Table I, application no. 17, has been published in A. And the activity of the gene product thereof is the activity of ypl144w-protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “ypl144w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said Ypl144w, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ypl144w, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ypl144w, or a functional equivalent or a homolog thereof as depicted in column 8 of Table II, application no. 17, preferably a homolog or functional equivalent as depicted in column 8 of Table II B, application no. 17, and being depicted in the same respective line as said Ypl144w, and preferably the activity is increased plastidic, whereby the respective line disclose in column 7 the fine chemical 3,4,5-trihydroxypentanoic acid.

Accordingly, in one embodiment, the molecule which activity is to be increased in the process of the invention is the gene product with an activity as a “ypl144w-protein”, preferably it is the molecule of section (a) or (b) of this paragraph.

In particular, it was observed that in plants, especially in Arabidopsis thaliana, increasing or generating the activity of a gene product plastidic with the activity of a “ypl144w-protein”, preferably being encoded by a gene comprising the nucleic acid sequence SEQ ID NO. 151927, preferably the coding region thereof, conferred the production of or the increase in 3,4,5-trihydroxypentanoic acid compared with the wild type control.

The nucleic acid sequence of Ypr098c from Saccharomyces cerevisiae, e.g. as shown in the respective line in column 5 of Table I, application no. 17, has been published in A. And the activity of the gene product thereof is the activity of ypr098c-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “ypr098c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Ypr098c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ypr098c, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ypr098c, or a functional equivalent or a homolog thereof as depicted in column 8 of Table II, application no. 17, preferably a homolog or functional equivalent as depicted in column 8 of Table II B, application no. 17, and being depicted in the same respective line as said Ypr098c, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “ypr098c-protein”, preferably it is the molecule of section (a) or (b) of this paragraph.

In particular, it was observed that in plants, especially in Arabidopsis thaliana, increasing or generating the activity of a gene product plastidic with the activity of a “ypr098c-protein”, preferably being encoded by a gene comprising the nucleic acid sequence SEQ ID NO. 151931, preferably the coding region thereof, conferred the production of or the increase in glucose-1-phosphate compared with the wild type control.

The nucleic acid sequence of Ypr098c from Saccharomyces cerevisiae, e.g. as shown in the respective line in column 5 of Table I, application no. 17, has been published in A. And the activity of the gene product thereof is the activity of ypr098c-protein.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “ypr098c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said Ypr098c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ypr098c, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ypr098c, or a functional equivalent or a homolog thereof as depicted in column 8 of Table II, application no. 17, preferably a homolog or functional equivalent as depicted in column 8 of Table II B, application no. 17, and being depicted in the same respective line as said Ypr098c, and preferably the activity is increased plastidic, whereby the respective line disclose in column 7 the fine chemical 3,4,5-trihydroxypentanoic acid.

Accordingly, in one embodiment, the molecule which activity is to be increased in the process of the invention is the gene product with an activity as a “ypr098c-protein”, preferably it is the molecule of section (a) or (b) of this paragraph.

In particular, it was observed that in plants, especially in Arabidopsis thaliana, increasing or generating the activity of a gene product plastidic with the activity of a “ypr098c-protein”, preferably being encoded by a gene comprising the nucleic acid sequence SEQ ID NO. 151931, preferably the coding region thereof, conferred the production of or the increase in 3,4,5-trihydroxypentanoic acid compared with the wild type control.

The nucleic acid sequence of Ypr156c from Saccharomyces cerevisiae, e.g. as shown in the respective line in column 5 of Table I, application no. 17, has been published in A. And the activity of the gene product thereof is the activity of polyamine transporter.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “polyamine transporter”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said Ypr156c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ypr156c, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ypr156c, or a functional equivalent or a homolog thereof as depicted in column 8 of Table II, application no. 17, preferably a homolog or functional equivalent as depicted in column 8 of Table II B, application no. 17, and being depicted in the same respective line as said Ypr156c, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “polyamine transporter”, preferably it is the molecule of section (a) or (b) of this paragraph.

In particular, it was observed that in plants, especially in Arabidopsis thaliana, increasing or generating the activity of a gene product plastidic with the activity of a “polyamine transporter”, preferably being encoded by a gene comprising the nucleic acid sequence SEQ ID NO. 151958, preferably the coding region thereof, conferred the production of or the increase in glucose-1-phosphate compared with the wild type control.

The nucleic acid sequence of Ypr156c from Saccharomyces cerevisiae, e.g. as shown in the respective line in column 5 of Table I, application no. 17, has been published in A. And the activity of the gene product thereof is the activity of polyamine transporter.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “polyamine transporter”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said Ypr156c, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Ypr156c, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Ypr156c, or a functional equivalent or a homolog thereof as depicted in column 8 of Table II, application no. 17, preferably a homolog or functional equivalent as depicted in column 8 of Table II B, application no. 17, and being depicted in the same respective line as said Ypr156c, and preferably the activity is increased plastidic, whereby the respective line disclose in column 7 the fine chemical myo-inositol.

Accordingly, in one embodiment, the molecule which activity is to be increased in the process of the invention is the gene product with an activity as a “polyamine transporter”, preferably it is the molecule of section (a) or (b) of this paragraph.

In particular, it was observed that in plants, especially in Arabidopsis thaliana, increasing or generating the activity of a gene product plastidic with the activity of a “polyamine transporter”, preferably being encoded by a gene comprising the nucleic acid sequence SEQ ID NO. 151958, preferably the coding region thereof, conferred the production of or the increase in myo-inositol compared with the wild type control.

The nucleic acid sequence of Zm_{—}4842_BE510522 from Zea mays, e.g. as shown in the respective line in column 5 of Table I, application no. 17, is unpublished. And the activity of the gene product thereof is the activity of Zm_{—}4842_BE510522-protein.

Accordingly, in one embodiment, the process of the present invention for producing fructose in a non-human organism, like a microorganism or a plant or a part thereof, comprises increasing or generating the activity of a gene product with the activity of a gene product conferring the activity of “Zm_{—}4842_BE510522-protein”, especially from Zea mays or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said Zm_{—}4842_BE510522, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Zm_{—}4842_BE510522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Zm_{—}4842_BE510522, or a functional equivalent or a homolog thereof as depicted in column 8 of Table II, application no. 17, preferably a homolog or functional equivalent as depicted in column 8 of Table II B, application no. 17, and being depicted in the same respective line as said Zm_{—}4842_BE510522, and preferably the activity is increased non-targeted, whereby the respective line disclose in column 7 the fine chemical fructose.

Accordingly, in one embodiment, the molecule which activity is to be increased in the process of the invention is the gene product with an activity as a “Zm_{—}4842_BE510522-protein”, preferably it is the molecule of section (a) or (b) of this paragraph.

In particular, it was observed that in plants, especially in Arabidopsis thaliana, increasing or generating the activity of a gene product non-targeted with the activity of a “Zm_{—}4842_BE510522-protein”, preferably being encoded by a gene comprising the nucleic acid sequence SEQ ID NO. 68413, preferably the coding region thereof, conferred the production of or the increase in fructose compared with the wild type control.

The nucleic acid sequence of Zm_{—}4842_BE510522 from Zea mays, e.g. as shown in the respective line in column 5 of Table I, application no. 17, is unpublished. And the activity of the gene product thereof is the activity of Zm_{—}4842_BE510522-protein.

Accordingly, in one embodiment, the process of the present invention for producing glucose in a non-human organism, like a microorganism or a plant or a part thereof, comprises increasing or generating the activity of a gene product with the activity of a gene product conferring the activity of “Zm_{—}4842_BE510522-protein”, especially from Zea mays or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said Zm_{—}4842_BE510522, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said Zm_{—}4842_BE510522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said Zm_{—}4842_BE510522, or a functional equivalent or a homolog thereof as depicted in column 8 of Table II, application no. 17, preferably a homolog or functional equivalent as depicted in column 8 of Table II B, application no. 17, and being depicted in the same respective line as said Zm_{—}4842_BE510522, and preferably the activity is increased non-targeted, whereby the respective line disclose in column 7 the fine chemical glucose.

Accordingly, in one embodiment, the molecule which activity is to be increased in the process of the invention is the gene product with an activity as a “Zm_{—}4842_BE510522-protein”, preferably it is the molecule of section (a) or (b) of this paragraph.

In particular, it was observed that in plants, especially in Arabidopsis thaliana, increasing or generating the activity of a gene product non-targeted with the activity of a “Zm_{—}4842_BE510522-protein”, preferably being encoded by a gene comprising the nucleic acid sequence SEQ ID NO. 68413, preferably the coding region thereof, conferred the production of or the increase in glucose compared with the wild type control.

The nucleic acid sequence of 44849766_CANOLA from Brassica napus, e.g. as shown in the respective line in column 5 of Table I, application no. 17, is unpublished. And the activity of the gene product thereof is the activity of acetyltransferase.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “acetyltransferase”, especially from Brassica napus or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said 44849766_CANOLA, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said 44849766_CANOLA, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said 44849766_CANOLA, or a functional equivalent or a homolog thereof as depicted in column 8 of Table II, application no. 17, preferably a homolog or functional equivalent as depicted in column 8 of Table II B, application no. 17, and being depicted in the same respective line as said 44849766_CANOLA, and preferably the activity is increased plastidic, whereby the respective line disclose in column 7 the fine chemical myo-inositol.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product plastidic with the activity of a “acetyltransferase”, preferably being encoded by a gene comprising the nucleic acid sequence SEQ ID NO. 140626, preferably the coding region thereof, conferred the production of or the increase in myo-inositol compared with the wild type control.

The nucleic acid sequence of 47266012_SOYBEAN from Glycine max, e.g. as shown in the respective line in column 5 of Table I, application no. 17, is unpublished. And the activity of the gene product thereof is the activity of 47266012-protein.

Accordingly, in one embodiment, the process of the present invention for producing fructose in a non-human organism, like a microorganism or a plant or a part thereof, comprises increasing or generating the activity of a gene product with the activity of a gene product conferring the activity of “47266012-protein”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said 47266012_SOYBEAN, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said 47266012_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said 47266012_SOYBEAN, or a functional equivalent or a homolog thereof as depicted in column 8 of Table II, application no. 17, preferably a homolog or functional equivalent as depicted in column 8 of Table II B, application no. 17, and being depicted in the same respective line as said 47266012_SOYBEAN, and preferably the activity is increased non-targeted, whereby the respective line disclose in column 7 the fine chemical fructose.

Accordingly, in one embodiment, the molecule which activity is to be increased in the process of the invention is the gene product with an activity as a “47266012-protein”, preferably it is the molecule of section (a) or (b) of this paragraph.

In particular, it was observed that in plants, especially in Arabidopsis thaliana, increasing or generating the activity of a gene product non-targeted with the activity of a “47266012-protein”, preferably being encoded by a gene comprising the nucleic acid sequence SEQ ID NO. 15187, preferably the coding region thereof, conferred the production of or the increase in fructose compared with the wild type control.

The nucleic acid sequence of 47266012_SOYBEAN from Glycine max, e.g. as shown in the respective line in column 5 of Table I, application no. 17, is unpublished. And the activity of the gene product thereof is the activity of 47266012-protein.

Accordingly, in one embodiment, the process of the present invention for producing glucose in a non-human organism, like a microorganism or a plant or a part thereof, comprises increasing or generating the activity of a gene product with the activity of a gene product conferring the activity of “47266012-protein”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said 47266012_SOYBEAN, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said 47266012_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said 47266012_SOYBEAN, or a functional equivalent or a homolog thereof as depicted in column 8 of Table II, application no. 17, preferably a homolog or functional equivalent as depicted in column 8 of Table II B, application no. 17, and being depicted in the same respective line as said 47266012_SOYBEAN, and preferably the activity is increased non-targeted, whereby the respective line disclose in column 7 the fine chemical glucose.

Accordingly, in one embodiment, the molecule which activity is to be increased in the process of the invention is the gene product with an activity as a “47266012-protein”, preferably it is the molecule of section (a) or (b) of this paragraph.

In particular, it was observed that in plants, especially in Arabidopsis thaliana, increasing or generating the activity of a gene product non-targeted with the activity of a “47266012-protein”, preferably being encoded by a gene comprising the nucleic acid sequence SEQ ID NO. 15187, preferably the coding region thereof, conferred the production of or the increase in glucose compared with the wild type control.

The nucleic acid sequence of 47266012_SOYBEAN from Glycine max, e.g. as shown in the respective line in column 5 of Table I, application no. 17, is unpublished. And the activity of the gene product thereof is the activity of 47266012-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “47266012-protein”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said 47266012_SOYBEAN, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said 47266012_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said 47266012_SOYBEAN, or a functional equivalent or a homolog thereof as depicted in column 8 of Table II, application no. 17, preferably a homolog or functional equivalent as depicted in column 8 of Table II B, application no. 17, and being depicted in the same respective line as said 47266012_SOYBEAN, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “47266012-protein”, preferably it is the molecule of section (a) or (b) of this paragraph.

In particular, it was observed that in plants, especially in Arabidopsis thaliana, increasing or generating the activity of a gene product non-targeted with the activity of a “47266012-protein”, preferably being encoded by a gene comprising the nucleic acid sequence SEQ ID NO. 15187, preferably the coding region thereof, conferred the production of or the increase in glucose-1-phosphate compared with the wild type control.

The nucleic acid sequence of 47266012_SOYBEAN from Glycine max, e.g. as shown in the respective line in column 5 of Table I, application no. 17, is unpublished. And the activity of the gene product thereof is the activity of 47266012-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “47266012-protein”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said 47266012_SOYBEAN, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said 47266012_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said 47266012_SOYBEAN, or a functional equivalent or a homolog thereof as depicted in column 8 of Table II, application no. 17, preferably a homolog or functional equivalent as depicted in column 8 of Table II B, application no. 17, and being depicted in the same respective line as said 47266012_SOYBEAN, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “47266012-protein”, preferably it is the molecule of section (a) or (b) of this paragraph.

In particular, it was observed that in plants, especially in Arabidopsis thaliana, increasing or generating the activity of a gene product non-targeted with the activity of a “47266012-protein”, preferably being encoded by a gene comprising the nucleic acid sequence SEQ ID NO. 15187, preferably the coding region thereof, conferred the production of or the increase in myo-inositol compared with the wild type control.

The nucleic acid sequence of 47266012_SOYBEAN from Glycine max, e.g. as shown in the respective line in column 5 of Table I, application no. 17, is unpublished. And the activity of the gene product thereof is the activity of 47266012-protein.

Accordingly, in one embodiment, the process of the present invention for producing sedoheptulose-7-phosphate in a non-human organism, like a microorganism or a plant or a part thereof, comprises increasing or generating the activity of a gene product with the activity of a gene product conferring the activity of “47266012-protein”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical sedoheptulose-7-phosphate), application no. 17, preferably the coding region thereof, or a homolog or a fragment thereof, and being depicted in the same respective line as said 47266012_SOYBEAN, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said 47266012_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said 47266012_SOYBEAN, or a functional equivalent or a homolog thereof as depicted in column 8 of Table II, application no. 17, preferably a homolog or functional equivalent as depicted in column 8 of Table II B, application no. 17, and being depicted in the same respective line as said 47266012_SOYBEAN, and preferably the activity is increased non-targeted, whereby the respective line disclose in column 7 the fine chemical sedoheptulose-7-phosphate.

Accordingly, in one embodiment, the molecule which activity is to be increased in the process of the invention is the gene product with an activity as a “47266012-protein”, preferably it is the molecule of section (a) or (b) of this paragraph.

In particular, it was observed that in plants, especially in Arabidopsis thaliana, increasing or generating the activity of a gene product non-targeted with the activity of a “47266012-protein”, preferably being encoded by a gene comprising the nucleic acid sequence SEQ ID NO. 15187, preferably the coding region thereof, conferred the production of or the increase in sedoheptulose-7-phosphate compared with the wild type control.

The nucleic acid sequence of 49747384_SOYBEAN from Glycine max, e.g. as shown in the respective line in column 5 of Table I, application no. 17, is unpublished. And the activity of the gene product thereof is the activity of 49747384_SOYBEAN-protein.

Accordingly, in one embodiment, the process of the present invention for producing glucose in a non-human organism, like a microorganism or a plant or a part thereof, comprises increasing or generating the activity of a gene product with the activity of a gene product conferring the activity of “49747384_SOYBEAN-protein”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said 49747384_SOYBEAN, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said 49747384_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said 49747384_SOYBEAN, or a functional equivalent or a homolog thereof as depicted in column 8 of Table II, application no. 17, preferably a homolog or functional equivalent as depicted in column 8 of Table II B, application no. 17, and being depicted in the same respective line as said 49747384_SOYBEAN, and preferably the activity is increased non-targeted, whereby the respective line disclose in column 7 the fine chemical glucose.

Accordingly, in one embodiment, the molecule which activity is to be increased in the process of the invention is the gene product with an activity as a “49747384_SOYBEAN-protein”, preferably it is the molecule of section (a) or (b) of this paragraph.

In particular, it was observed that in plants, especially in Arabidopsis thaliana, increasing or generating the activity of a gene product non-targeted with the activity of a “49747384_SOYBEAN-protein”, preferably being encoded by a gene comprising the nucleic acid sequence SEQ ID NO. 69, preferably the coding region thereof, conferred the production of or the increase in glucose compared with the wild type control.

The nucleic acid sequence of 49747384_SOYBEAN from Glycine max, e.g. as shown in the respective line in column 5 of Table I, application no. 17, is unpublished. And the activity of the gene product thereof is the activity of 49747384_SOYBEAN-protein.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “49747384_SOYBEAN-protein”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said 49747384_SOYBEAN, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said 49747384_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said 49747384_SOYBEAN, or a functional equivalent or a homolog thereof as depicted in column 8 of Table II, application no. 17, preferably a homolog or functional equivalent as depicted in column 8 of Table II B, application no. 17, and being depicted in the same respective line as said 49747384_SOYBEAN, and preferably the activity is increased non-targeted, whereby the respective line disclose in column 7 the fine chemical glucose-1-phosphate.

Accordingly, in one embodiment, the molecule which activity is to be increased in the process of the invention is the gene product with an activity as a “49747384_SOYBEAN-protein”, preferably it is the molecule of section (a) or (b) of this paragraph.

In particular, it was observed that in plants, especially in Arabidopsis thaliana, increasing or generating the activity of a gene product non-targeted with the activity of a “49747384_SOYBEAN-protein”, preferably being encoded by a gene comprising the nucleic acid sequence SEQ ID NO. 69, preferably the coding region thereof, conferred the production of or the increase in glucose-1-phosphate compared with the wild type control.

The nucleic acid sequence of 49747384_SOYBEAN from Glycine max, e.g. as shown in the respective line in column 5 of Table I, application no. 17, is unpublished. And the activity of the gene product thereof is the activity of 49747384_SOYBEAN-protein.

Accordingly, in one embodiment, the process of the present invention for producing myo-inositol in a non-human organism, like a microorganism or a plant or a part thereof, comprises increasing or generating the activity of a gene product with the activity of a gene product conferring the activity of “49747384_SOYBEAN-protein”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical myo-inositol), application no. 17, preferably the coding region thereof, or a homolog or a fragment thereof, and being depicted in the same respective line as said 49747384_SOYBEAN, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said 49747384_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said 49747384_SOYBEAN, or a functional equivalent or a homolog thereof as depicted in column 8 of Table II, application no. 17, preferably a homolog or functional equivalent as depicted in column 8 of Table II B, application no. 17, and being depicted in the same respective line as said 49747384_SOYBEAN, and preferably the activity is increased non-targeted, whereby the respective line disclose in column 7 the fine chemical myo-inositol.

Accordingly, in one embodiment, the molecule which activity is to be increased in the process of the invention is the gene product with an activity as a “49747384_SOYBEAN-protein”, preferably it is the molecule of section (a) or (b) of this paragraph.

In particular, it was observed that in plants, especially in Arabidopsis thaliana, increasing or generating the activity of a gene product non-targeted with the activity of a “49747384_SOYBEAN-protein”, preferably being encoded by a gene comprising the nucleic acid sequence SEQ ID NO. 69, preferably the coding region thereof, conferred the production of or the increase in myo-inositol compared with the wild type control.

The nucleic acid sequence of 49747384_SOYBEAN from Glycine max, e.g. as shown in the respective line in column 5 of Table I, application no. 17, is unpublished. And the activity of the gene product thereof is the activity of 49747384_SOYBEAN-protein.

Accordingly, in one embodiment, the process of the present invention for producing raffinose in a non-human organism, like a microorganism or a plant or a part thereof, comprises increasing or generating the activity of a gene product with the activity of a gene product conferring the activity of “49747384_SOYBEAN-protein”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical raffinose), application no. 17, preferably the coding region thereof, or a homolog or a fragment thereof, and being depicted in the same respective line as said 49747384_SOYBEAN, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said 49747384_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said 49747384_SOYBEAN, or a functional equivalent or a homolog thereof as depicted in column 8 of Table II, application no. 17, preferably a homolog or functional equivalent as depicted in column 8 of Table II B, application no. 17, and being depicted in the same respective line as said 49747384_SOYBEAN, and preferably the activity is increased non-targeted, whereby the respective line disclose in column 7 the fine chemical raffinose.

Accordingly, in one embodiment, the molecule which activity is to be increased in the process of the invention is the gene product with an activity as a “49747384_SOYBEAN-protein”, preferably it is the molecule of section (a) or (b) of this paragraph.

In particular, it was observed that in plants, especially in Arabidopsis thaliana, increasing or generating the activity of a gene product non-targeted with the activity of a “49747384_SOYBEAN-protein”, preferably being encoded by a gene comprising the nucleic acid sequence SEQ ID NO. 69, preferably the coding region thereof, conferred the production of or the increase in raffinose compared with the wild type control.

The nucleic acid sequence of 49747384_SOYBEAN from Glycine max, e.g. as shown in the respective line in column 5 of Table I, application no. 17, is unpublished. And the activity of the gene product thereof is the activity of 49747384_SOYBEAN-protein.

Accordingly, in one embodiment, the process of the present invention for producing sedoheptulose-7-phosphate in a non-human organism, like a microorganism or a plant or a part thereof, comprises increasing or generating the activity of a gene product with the activity of a gene product conferring the activity of “49747384_SOYBEAN-protein”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical sedoheptulose-7-phosphate), application no. 17, preferably the coding region thereof, or a homolog or a fragment thereof, and being depicted in the same respective line as said 49747384_SOYBEAN, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said 49747384_SOYBEAN, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said 49747384_SOYBEAN, or a functional equivalent or a homolog thereof as depicted in column 8 of Table II, application no. 17, preferably a homolog or functional equivalent as depicted in column 8 of Table II B, application no. 17, and being depicted in the same respective line as said 49747384_SOYBEAN, and preferably the activity is increased non-targeted, whereby the respective line disclose in column 7 the fine chemical sedoheptulose-7-phosphate.

Accordingly, in one embodiment, the molecule which activity is to be increased in the process of the invention is the gene product with an activity as a “49747384_SOYBEAN-protein”, preferably it is the molecule of section (a) or (b) of this paragraph.

In particular, it was observed that in plants, especially in Arabidopsis thaliana, increasing or generating the activity of a gene product non-targeted with the activity of a “49747384_SOYBEAN-protein”, preferably being encoded by a gene comprising the nucleic acid sequence SEQ ID NO. 69, preferably the coding region thereof, conferred the production of or the increase in sedoheptulose-7-phosphate compared with the wild type control.

The nucleic acid sequence of 59582753_SOYBEAN from Glycine max, e.g. as shown in the respective line in column 5 of Table I, application no. 17, is unpublished. And the activity of the gene product thereof is the activity of glucose-6-phosphate 1-dehydrogenase.

Accordingly, in one embodiment, the process of the present invention for producing fructose in a non-human organism, like a microorganism or a plant or a part thereof, comprises increasing or generating the activity of a gene product with the activity of a gene product conferring the activity of “glucose-6-phosphate 1-dehydrogenase”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said 59582753_SOYBEAN, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said 59582753_SOYBEAN, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said 59582753_SOYBEAN, or a functional equivalent or a homolog thereof as depicted in column 8 of Table II, application no. 17, preferably a homolog or functional equivalent as depicted in column 8 of Table II B, application no. 17, and being depicted in the same respective line as said 59582753_SOYBEAN, and preferably the activity is increased plastidic, whereby the respective line disclose in column 7 the fine chemical fructose.

Accordingly, in one embodiment, the molecule which activity is to be increased in the process of the invention is the gene product with an activity as a “glucose-6-phosphate 1-dehydrogenase”, preferably it is the molecule of section (a) or (b) of this paragraph.

In particular, it was observed that in plants, especially in Arabidopsis thaliana, increasing or generating the activity of a gene product plastidic with the activity of a “glucose-6-phosphate 1-dehydrogenase”, preferably being encoded by a gene comprising the nucleic acid sequence SEQ ID NO. 16883, preferably the coding region thereof, conferred the production of or the increase in fructose compared with the wild type control.

The nucleic acid sequence of 59582753_SOYBEAN from Glycine max, e.g. as shown in the respective line in column 5 of Table I, application no. 17, is unpublished. And the activity of the gene product thereof is the activity of glucose-6-phosphate 1-dehydrogenase.

Accordingly, in one embodiment, the process of the present invention for producing glucose in a non-human organism, like a microorganism or a plant or a part thereof, comprises increasing or generating the activity of a gene product with the activity of a gene product conferring the activity of “glucose-6-phosphate 1-dehydrogenase”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said 59582753_SOYBEAN, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said 59582753_SOYBEAN, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said 59582753_SOYBEAN, or a functional equivalent or a homolog thereof as depicted in column 8 of Table II, application no. 17, preferably a homolog or functional equivalent as depicted in column 8 of Table II B, application no. 17, and being depicted in the same respective line as said 59582753_SOYBEAN, and preferably the activity is increased plastidic, whereby the respective line disclose in column 7 the fine chemical glucose.

Accordingly, in one embodiment, the molecule which activity is to be increased in the process of the invention is the gene product with an activity as a “glucose-6-phosphate 1-dehydrogenase”, preferably it is the molecule of section (a) or (b) of this paragraph.

In particular, it was observed that in plants, especially in Arabidopsis thaliana, increasing or generating the activity of a gene product plastidic with the activity of a “glucose-6-phosphate 1-dehydrogenase”, preferably being encoded by a gene comprising the nucleic acid sequence SEQ ID NO. 16883, preferably the coding region thereof, conferred the production of or the increase in glucose compared with the wild type control.

The nucleic acid sequence of 59582753_SOYBEAN from Glycine max, e.g. as shown in the respective line in column 5 of Table I, application no. 17, is unpublished. And the activity of the gene product thereof is the activity of glucose-6-phosphate 1-dehydrogenase.

Accordingly, in one embodiment, the process of the present invention for producing glucose-1-phosphate in a non-human organism, like a microorganism or a plant or a part thereof, comprises increasing or generating the activity of a gene product with the activity of a gene product conferring the activity of “glucose-6-phosphate 1-dehydrogenase”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical glucose-1-phosphate), application no. 17, preferably the coding region thereof, or a homolog or a fragment thereof, and being depicted in the same respective line as said 59582753_SOYBEAN, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said 59582753_SOYBEAN, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said 59582753_SOYBEAN, or a functional equivalent or a homolog thereof as depicted in column 8 of Table II, application no. 17, preferably a homolog or functional equivalent as depicted in column 8 of Table II B, application no. 17, and being depicted in the same respective line as said 59582753_SOYBEAN, and preferably the activity is increased plastidic, whereby the respective line disclose in column 7 the fine chemical glucose-1-phosphate.

Accordingly, in one embodiment, the molecule which activity is to be increased in the process of the invention is the gene product with an activity as a “glucose-6-phosphate 1-dehydrogenase”, preferably it is the molecule of section (a) or (b) of this paragraph.

In particular, it was observed that in plants, especially in Arabidopsis thaliana, increasing or generating the activity of a gene product plastidic with the activity of a “glucose-6-phosphate 1-dehydrogenase”, preferably being encoded by a gene comprising the nucleic acid sequence SEQ ID NO. 16883, preferably the coding region thereof, conferred the production of or the increase in glucose-1-phosphate compared with the wild type control.

The nucleic acid sequence of 59582753_SOYBEAN from Glycine max, e.g. as shown in the respective line in column 5 of Table I, application no. 17, is unpublished. And the activity of the gene product thereof is the activity of glucose-6-phosphate 1-dehydrogenase.

Accordingly, in one embodiment, the process of the present invention for producing sedoheptulose-7-phosphate in a non-human organism, like a microorganism or a plant or a part thereof, comprises increasing or generating the activity of a gene product with the activity of a gene product conferring the activity of “glucose-6-phosphate 1-dehydrogenase”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical sedoheptulose-7-phosphate), application no. 17, preferably the coding region thereof, or a homolog or a fragment thereof, and being depicted in the same respective line as said 59582753_SOYBEAN, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said 59582753_SOYBEAN, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said 59582753_SOYBEAN, or a functional equivalent or a homolog thereof as depicted in column 8 of Table II, application no. 17, preferably a homolog or functional equivalent as depicted in column 8 of Table II B, application no. 17, and being depicted in the same respective line as said 59582753_SOYBEAN, and preferably the activity is increased plastidic, whereby the respective line disclose in column 7 the fine chemical sedoheptulose-7-phosphate.

Accordingly, in one embodiment, the molecule which activity is to be increased in the process of the invention is the gene product with an activity as a “glucose-6-phosphate 1-dehydrogenase”, preferably it is the molecule of section (a) or (b) of this paragraph.

In particular, it was observed that in plants, especially in Arabidopsis thaliana, increasing or generating the activity of a gene product plastidic with the activity of a “glucose-6-phosphate 1-dehydrogenase”, preferably being encoded by a gene comprising the nucleic acid sequence SEQ ID NO. 16883, preferably the coding region thereof, conferred the production of or the increase in sedoheptulose-7-phosphate compared with the wild type control.

The nucleic acid sequence of 59582753_SOYBEAN from Glycine max, e.g. as shown in the respective line in column 5 of Table I, application no. 17, is unpublished. And the activity of the gene product thereof is the activity of glucose-6-phosphate 1-dehydrogenase.

Accordingly, in one embodiment, the process of the present invention for producing sucrose in a non-human organism, like a microorganism or a plant or a part thereof, comprises increasing or generating the activity of a gene product with the activity of a gene product conferring the activity of “glucose-6-phosphate 1-dehydrogenase”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical sucrose), application no. 17, preferably the coding region thereof, or a homolog or a fragment thereof, and being depicted in the same respective line as said 59582753_SOYBEAN, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said 59582753_SOYBEAN, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said 59582753_SOYBEAN, or a functional equivalent or a homolog thereof as depicted in column 8 of Table II, application no. 17, preferably a homolog or functional equivalent as depicted in column 8 of Table II B, application no. 17, and being depicted in the same respective line as said 59582753_SOYBEAN, and preferably the activity is increased plastidic, whereby the respective line disclose in column 7 the fine chemical sucrose.

Accordingly, in one embodiment, the molecule which activity is to be increased in the process of the invention is the gene product with an activity as a “glucose-6-phosphate 1-dehydrogenase”, preferably it is the molecule of section (a) or (b) of this paragraph.

In particular, it was observed that in plants, especially in Arabidopsis thaliana, increasing or generating the activity of a gene product plastidic with the activity of a “glucose-6-phosphate 1-dehydrogenase”, preferably being encoded by a gene comprising the nucleic acid sequence SEQ ID NO. 16883, preferably the coding region thereof, conferred the production of or the increase in sucrose compared with the wild type control.

The nucleic acid sequence of 59582753_SOYBEAN from Glycine max, e.g. as shown in the respective line in column 5 of Table I, application no. 17, is unpublished. And the activity of the gene product thereof is the activity of glucose-6-phosphate 1-dehydrogenase.

Accordingly, in one embodiment, the process of the present invention for producing 3,4,5-trihydroxypentanoic acid in a non-human organism, like a microorganism or a plant or a part thereof, comprises increasing or generating the activity of a gene product with the activity of a gene product conferring the activity of “glucose-6-phosphate 1-dehydrogenase”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical 3,4,5-trihydroxypentanoic acid), application no. 17, preferably the coding region thereof, or a homolog or a fragment thereof, and being depicted in the same respective line as said 59582753_SOYBEAN, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said 59582753_SOYBEAN, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said 59582753_SOYBEAN, or a functional equivalent or a homolog thereof as depicted in column 8 of Table II, application no. 17, preferably a homolog or functional equivalent as depicted in column 8 of Table II B, application no. 17, and being depicted in the same respective line as said 59582753_SOYBEAN, and preferably the activity is increased plastidic, whereby the respective line disclose in column 7 the fine chemical 3,4,5-trihydroxypentanoic acid.

Accordingly, in one embodiment, the molecule which activity is to be increased in the process of the invention is the gene product with an activity as a “glucose-6-phosphate 1-dehydrogenase”, preferably it is the molecule of section (a) or (b) of this paragraph.

In particular, it was observed that in plants, especially in Arabidopsis thaliana, increasing or generating the activity of a gene product plastidic with the activity of a “glucose-6-phosphate 1-dehydrogenase”, preferably being encoded by a gene comprising the nucleic acid sequence SEQ ID NO. 16883, preferably the coding region thereof, conferred the production of or the increase in 3,4,5-trihydroxypentanoic acid compared with the wild type control.

The nucleic acid sequence of YJL072C from Saccharomyces cerevisiae, e.g. as shown in the respective line in column 5 of Table I, application no. 17, has been published in A. And the activity of the gene product thereof is the activity of DNA replication complex GINS protein.

Accordingly, in one embodiment, the process of the present invention for producing fructose in a non-human organism, like a microorganism or a plant or a part thereof, comprises increasing or generating the activity of a gene product with the activity of a gene product conferring the activity of “DNA replication complex GINS protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical fructose), application no. 17, preferably the coding region thereof, or a homolog or a fragment thereof, and being depicted in the same respective line as said YJL072C, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said YJL072C, and preferably the activity is increased non-targeted, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said YJL072C, or a functional equivalent or a homolog thereof as depicted in column 8 of Table II, application no. 17, preferably a homolog or functional equivalent as depicted in column 8 of Table II B, application no. 17, and being depicted in the same respective line as said YJL072C, and preferably the activity is increased non-targeted, whereby the respective line disclose in column 7 the fine chemical fructose.

Accordingly, in one embodiment, the molecule which activity is to be increased in the process of the invention is the gene product with an activity as a “DNA replication complex GINS protein”, preferably it is the molecule of section (a) or (b) of this paragraph.

In particular, it was observed that in plants, especially in Arabidopsis thaliana, increasing or generating the activity of a gene product non-targeted with the activity of a “DNA replication complex GINS protein”, preferably being encoded by a gene comprising the nucleic acid sequence SEQ ID NO. 149528, preferably the coding region thereof, conferred the production of or the increase in fructose compared with the wild type control.

The nucleic acid sequence of YLR153C from Saccharomyces cerevisiae, e.g. as shown in the respective line in column 5 of Table I, application no. 17, has been published in A. And the activity of the gene product thereof is the activity of acetyl CoA synthetase.

Accordingly, in one embodiment, the process of the present invention for producing glucose in a non-human organism, like a microorganism or a plant or a part thereof, comprises increasing or generating the activity of a gene product with the activity of a gene product conferring the activity of “acetyl CoA synthetase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glucose), application no. 17, preferably the coding region thereof, or a homolog or a fragment thereof, and being depicted in the same respective line as said YLR153C, or a functional equivalent or a homolog thereof as shown in column 8 of Table I, application no. 17, preferably the coding region thereof, particularly a homolog or functional equivalent as shown in column 8 of Table I B, application no. 17, and being depicted in the same respective line as said YLR153C, and preferably the activity is increased plastidic, or
(b) a polypeptide comprising a polypeptide, a consensus sequence or at least a polypeptide motif as shown in the respective line in column 5 of Table II or in column 8 of Table IV, application no. 17, respectively, and being depicted in the same respective line as said YLR153C, or a functional equivalent or a homolog thereof as depicted in column 8 of Table II, application no. 17, preferably a homolog or functional equivalent as depicted in column 8 of Table II B, application no. 17, and being depicted in the same respective line as said YLR153C, and preferably the activity is increased plastidic, whereby the respective line disclose in column 7 the fine chemical glucose.

Accordingly, in one embodiment, the molecule which activity is to be increased in the process of the invention is the gene product with an activity as a “acetyl CoA synthetase”, preferably it is the molecule of section (a) or (b) of this paragraph.

In particular, it was observed that in plants, especially in Arabidopsis thaliana, increasing or generating the activity of a gene product plastidic with the activity of a “acetyl CoA synthetase”, preferably being encoded by a gene comprising the nucleic acid sequence SEQ ID NO. 150960, preferably the coding region thereof, conferred the production of or the increase in glucose compared with the wild type control.

[0105.1.7.17] to [0107.1.7.17] for the disclosure of these paragraphs see [0105.1.7.7] to [0107.1.7.7] above

A protein having an activity conferring an increase in the amount or level of the fine chemical 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose, upon targeting to the plastids or mitochondria or upon non-targeting, preferably has the structure of the respective polypeptide described herein, in particular of the polypeptides comprising the consensus sequence or at least one polypeptide motifs as shown in the respective line in Table IV, application no. 17, column 8 or of the polypeptide comprising an amino acid sequence as disclosed in the respective line in Table II, application no. 17, columns 5 or 8, or homologs or fragments thereof as described herein, or is encoded by the nucleic acid molecule characterized herein or the nucleic acid molecule according to the invention, for example by the nucleic acid molecule as shown in the respective line in Table I, application no. 17, columns 5 or 8, preferably the coding region thereof, or homologs or fragments thereof and has the herein mentioned activity.

for the disclosure of this paragraph see [0109.1.7.7] above

for the disclosure of this paragraph see [0110.1.7.7] above

In one embodiment, the process of the present invention comprises one or more of the following steps
(a) stabilizing a protein conferring the generation or increased expression of a protein encoded by the nucleic acid molecule of the invention or of the polypeptide of the invention, e.g. of a polypeptide having the activity selected from the group consisting of 1,4-alpha-glucan branching enzyme, 2,3-dihydroxy-2,3-dihydrophenylpropionatedehydrogenase, 2-C-methyl-D-erythritol 4-phosphate cytidylyltransferase, 2-oxoglutarate dehydrogenase E1 subunit, 3-deoxy-7-phosphoheptulonate synthase, 3-isopropylmalate dehydratase large subunit, 3-phosphoglycerate dehydrogenase, 47266012-protein, 49747384_SOYBEAN-protein, 4-alpha-glucanotransferase, ABC transporter permease protein, acetyl CoA carboxylase, acetyl CoA synthetase, acetyl-coenzyme A synthetase, acetyltransferase, acid shock protein, acyl carrier protein, acyl transferase, acyl-CoA dehydrogenase, acyl-CoA synthase, adenylosuccinate lyase, alanine dehydrogenase, alcohol dehydrogenase, aldolase, alkaline phosphatase, amine oxidase, amino-acid acetyltransferase, aminotransferase, arginine exporter protein, asparagine synthetase B, AO g19350-protein, At2g45420-protein, At5g16650-protein, At5g43630-protein, ATP synthase subunit E, ATP-binding component of a transport system, auxin response factor, AX653549-protein, AY087308-protein, b0050-protein, b0251-protein, b0362-protein, b0488-protein, B0644-protein, b0801-protein, b0970-protein, b1003-protein, b1046-protein, b1137-protein, b1214-protein, b1219-protein, b1234-protein, b1285-protein, b1357-protein, b1410-protein, b1425-protein, b1445-protein, b1470-protein, b1522-protein, b1556-protein, b1583-protein, b1670-protein, b1796-protein, b1898-protein, b2032-protein, b2107-protein, b2139-protein, b2345-protein, b2360-protein, b2361-protein, b2399-protein, b2466-protein, b2474-protein, b2613-protein, b2653-protein, b2654-protein, b2670-protein, b2673-protein, b2682-protein, b2739-protein, b2932-protein, b3151-protein, b3221-protein, b3246-protein, b3346-protein, B3427-protein, b3442-protein, B3443-protein, b3777-protein, b3817-protein, b3818-protein, b3989-protein, b4029-protein, b4050-protein, b4056-protein, b4250-protein, beta-galactosidase, beta-hydroxylase, betaine aldehyde dehydrogenase, bifunctional protein (phosphoribosyltransferase and regulatory protein), binding-protein-dependent transport systems inner membrane component, branched-chain amino acid ABC transporter permease protein, branched-chain amino acid permease, calcium-dependent protein kinase, carbamoyl-phosphate synthase subunit, cardiolipin synthase, cation-transporting ATPase, CBL-interacting protein kinase, CCAAT-binding transcription factor, CDP-diglyceride synthetase, cell division control protein, cell division protein, chlorophyllase, chorismate lyase, circadian clock protein, colanic acid biosynthesis protein, coproporphyrinogen III oxidase, coproporphyrinogen oxidase, CTP synthase, cullin, cyclin, cyclin D, cystathionine gamma-synthase, cysteine synthase A, dATP pyrophosphohydrolase, dihydrolipoamide dehydrogenase, DNA binding protein, DNA replication complex GINS protein, DnaJ-like chaperone, electron transfer flavoprotein subunit beta, electron transport complex protein, elongation factor Tu, enoyl-CoA hydratase, eukaryotic translation initiation factor, eukaryotic translation initiation factor 5, fatty acid desaturase, ferredoxin, Fe—S subunit of oxidoreductase, flagellar M-ring protein, flavodoxin, fructose biphosphate aldolase, fumarylacetoacetate hydrolase, galactinol synthase, GDP-mannose 4,6-dehydratase, GDP-mannose dehydrogenase, geranylgeranyl pyrophosphate synthase, gibberellin 20-oxidase, Glu/Leu/PheNal dehydrogenase, glucokinase, gluconate transport system permease 3, glucose dehydrogenase, glucose-6-phosphate 1-dehydrogenase, glutamate-ammonia-ligase, glutamine amidotransferase, glutamine synthetase, glutaredoxin, glycerate kinase, glycogen (starch) synthase, glycogen synthase, glycosidase, glycosyl transferase, glycosyltransferase, GM02LC38418-protein, GM02LC46-protein, halotolerance protein, heat shock transcription factor, hexokinase, hexose transporter, high-affinity branched-chain amino acid transport protein, histone acetyltransferase, homocitrate synthase, hydrolase, inositol-3-phosphate synthase, integral membrane protein, intracellular septation protein, ion-transport protein, iron(III) dicitrate-binding protein, isochorismate synthase, isocitrate dehydrogenase, isopropylmalate isomerase large subunit, ketol-acid reductoisomerase, kinase, leucyl/phenylalanyl-tRNA-protein transferase, lipoprotein precursor, L-ribulose-5-phosphate 4-epimerase, L-serine dehydratase, lysine decarboxylase, MADS box protein transcription factor, MADS-box transcription factor, malate dehydrogenase, mannitol-1-phosphate dehydrogenase, membrane protein, membrane transport protein, methylglyoxalase, methyltransferase, Mg-protoporphyrin IX monomethyl ester oxidative cyclase 66 kD subunit, mitogen-activated protein kinase, molecular chaperone, monothiol glutaredoxin, monthiol glutaredoxin, multidrug resistance protein, multiple antibiotic resistance protein, murein transglycosylase, NAD(P)H dehydrogenase (quinone), NADH dehydrogenase, NADH-quinone oxidoreductase subunit, O-acetyltransferase, O-antigen chain length determinant, outer membrane receptor protein precursor, oxidoreductase, oxidoreductase subunit, para-aminobenzoate synthase component I, penicillin-binding protein, peptidyl-prolyl cis-trans isomerase, permease, permease protein of ABC transporter, phage-related repressor protein, phenylacetic acid degradation protein, phenylalanine ammonia-lyase, phosphate acetyltransferase, phosphate permease, phosphatidylinositol 3- and 4-kinase family protein, phosphoadenosine phosphosulfate reductase, phosphoanhydride phosphorylase, phosphoenolpyruvate carboxykinase, phosphoglycerate kinase, phosphohydrolase, phosphotransferase system component, photosystem I reaction center subunit XI, photosystem I reaction centre subunit, photosystem II protein, polyamine transporter, precorrin methylase, protease, protein kinase, protein phosphatase, protein-L-isoaspartate O-methyltransferase, purine biosynthesis protein, purine nucleoside phosphorylase, purine-nucleoside phosphorylase, putative transport system permease protein, putrescine transport system permease protein, pyridoxal phosphate biosynthetic protein, pyrimidine-specific ribonucleoside hydrolase, pyruvate kinase, recombinase A, RNA-binding protein, sec-independent protein translocase, Sec-independent protein translocase subunit, sensor histidine kinase, serine protease, serine/threonine dehydratase, serine/threonine-protein phosphatase, short chain dehydrogenase, short-chain alcohol dehydrogenase family, sll0254-protein, sll0281-protein, sll0354-protein, sll0418-protein, sll1761-protein, slr0600-protein, slr1107-protein, sn-glycerol-3-phosphate dehydrogenase, sn-glycerol-3-phosphate transport system permease protein, sodium/proton antiporter, sterol O-acyltransferase, subunit of COMPASS-protein complex, succinyl-CoA synthetase beta chain, ThiF family protein, thioredoxin, thioredoxin family protein, threonine dehydratase, threonine efflux protein, threonine synthase, transcription factor, transcriptional regulator, transcriptional regulator protein, transport protein, trehalase, trehalose-6-phosphate synthase, trehalose-phosphatase, tryptophan biosynthesis protein, TTC1386-protein, urease accessory protein, urease subunit, vacuolar sorting protein, XM_{—}473199-protein, ycl027w-protein, YCR047W-A-protein, ycr102c-protein, YDL159W-A-protein, ydl235c-protein, ydr183w-protein, ydr507c-protein, YEL030C-A-protein, yer177w-protein, YFL019C-protein, ygl096w-protein, ygl237c-protein, yhl013c-protein, YIL083C-protein, YJL127W-A-protein, ymr324c-protein, yol160w-protein, ypl144w-protein, ypr098c-protein, zinc transporter, and Zm_{—}4842_BE510522-protein, or of a polypeptide as indicated in the respective line in Table II, application no. 17, columns 5 or 8, or its homologs or fragments, and conferring the production of or an increase in 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose, respectively; and/or
(b) stabilizing a mRNA conferring the generation or increased expression of a FCRP, e.g. protein encoded by the nucleic acid molecule of the invention or its homologs or fragments, or of a mRNA encoding the polypeptide of the present invention having the hereinmentioned activity selected from the group consisting of said activities mentioned in (a) and conferring the production of or an increase in 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose, respectively; and/or
(c) increasing the specific activity of a protein conferring the increased expression of a FCRP, e.g. a protein encoded by the nucleic acid molecule of the invention or of the polypeptide of the present invention having herein-mentioned 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose generating or increasing activity, respectively, e.g. of a polypeptide having the activity of a protein as indicated in the respective line in Table II, application no. 17, columns 5 or 8, or its homologs or fragments, or decreasing the inhibitory regulation of the polypeptide of the invention; and/or
(d) generating or increasing the expression of an endogenous or artificial transcription factor mediating the expression of a protein conferring the generation or increased expression of a FCRP, e.g. a protein encoded by the nucleic acid molecule of the invention or of the polypeptide of the invention having herein-mentioned activity selected from the group consisting of said activities mentioned in (a) and conferring a 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose increasing activity, respectively, e.g. of a polypeptide having the activity of a protein as indicated in the respective line in Table II, application no. 17, columns 5 or 8 or its homologs; and/or
(e) stimulating activity of a protein conferring the increased expression of a FCRP, e.g. a protein encoded by the nucleic acid molecule of the present invention or a polypeptide of the present invention having herein-mentioned activity selected from the group consisting of said activities mentioned in (a) and conferring a 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose increasing activity, respectively, e.g. of a polypeptide having the activity of a protein as indicated in the respective line in Table II, application no. 17, columns 5 or 8, or its homologs or fragments, by adding one or more exogenous inducing factors to the non-human organism or parts thereof; and/or
(f) expressing a transgenic gene encoding a protein conferring the increased expression of a FCRP, e.g. a polypeptide encoded by the nucleic acid molecule of the present invention or a polypeptide of the present invention, having herein-mentioned activity selected from the group consisting of said activities mentioned in (a) and conferring a 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose increasing activity, respectively, e.g. of a polypeptide having the activity of a protein as indicated in the respective line in Table II, application no. 17, columns 5 or 8, or its homologs or fragments, and/or
(g) increasing the copy number of a gene conferring the increased expression of a nucleic acid molecule encoding a FCRP, e.g. a polypeptide encoded by the nucleic acid molecule of the invention or the polypeptide of the invention having herein-mentioned activity selected from the group consisting of said activities mentioned in (a) and conferring a 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose increasing activity, respectively, e.g. of a polypeptide having the activity of a protein as indicated in the respective line in Table II, application no. 17, columns 5 or 8, or its homologs or fragments; and/or
(h) increasing the expression of the endogenous gene encoding the FCRP, polypeptide of the invention, e.g. a polypeptide encoded by the nucleic acid molecule of the invention or the polypeptide of the invention having herein-mentioned activity selected from the group consisting of said activities mentioned in (a) and conferring a 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose; increasing activity, respectively, e.g. of a polypeptide having the activity of a protein as indicated in the respective line in Table II, application no. 17, columns 5 or 8, or its homologs or fragments, by adding positive expression or removing negative expression elements, e.g. homologous recombination can be used to either introduce positive regulatory elements like for plants the 35S enhancer into the promoter or to remove repressor elements form regulatory regions. Further gene conversion methods can be used to disrupt repressor elements or to enhance to activity of positive elements. Positive elements can be randomly introduced in plants by T-DNA or transposon mutagenesis and lines can be identified in which the positive elements have be integrated near to a gene of the invention, the expression of which is thereby enhanced; and/or
(i) modulating growth conditions of the non-human organism in such a manner, that the expression or activity of the gene encoding the FCRP, e.g. a protein of the invention or the protein itself is enhanced, for example microorganisms or plants can be grown for example under a higher temperature regime leading to an enhanced expression of heat shock proteins, which can lead to an enhanced 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose production, respectively; and/or
(j) selecting of non-human organisms with especially high activity of the proteins of the invention from natural or from mutagenized resources and breeding them into the target organisms, e.g. the elite crops; and/or
(k) directing a protein encoded by the nucleic acid molecule of the invention or of the polypeptide of the present invention having herein-mentioned activity selected from the group consisting of said activities mentioned in (a) and conferring a 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose increasing activity, respectively, e.g. of a polypeptide having the activity of a protein as indicated in the respective line in Table II, application no. 17, columns 5 or 8, or its homologs or fragments, if for the corresponding nucleic acid molecule in the respective line in column 6 of Table I the term “plastidic” is indicated, to the plastids by the addition of a plastidial targeting sequence or if for the corresponding nucleic acid molecule in the respective line in column 6 of Table I the term “mitochondric” is indicated, to the mitochondria by the addition of a mitochondrial targeting sequence; and/or
(l) generating the expression of a protein encoded by the nucleic acid molecule of the invention or of the polypeptide of the present invention having herein-mentioned activity selected from the group consisting of said activities mentioned in (a) and conferring a 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose increasing activity, respectively, e.g. of a polypeptide having the activity of a protein as indicated in the respective line in Table II, application no. 17, columns 5 or 8, or its homologs or fragments, if for the corresponding nucleic acid molecule in the respective line in column 6 of Table I the term “plastidic” or “mitochondric” is indicated, in these organelles by the stable or transient transformation, advantageously stable transformation, of organelles, preferably plastids or mitochondria, with an inventive nucleic acid sequence preferably in form of an expression cassette containing said sequence leading to the expression of the nucleic acids or polypeptides of the invention in the respective organelle; and/or
(m) generating the expression of a protein encoded by the nucleic acid molecule of the invention or of the polypeptide of the present invention having herein-mentioned activity selected from the group consisting of said activities mentioned in (a) and conferring a 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, and/or verbascose increasing activity, respectively, e.g. of a polypeptide having the activity of a protein as indicated in the respective line in Table II, application no. 17, columns 5 or 8, or its homologs or fragments, if for the corresponding nucleic acid molecule in the respective line in column 6 of Table I the term “plastidic” or “mitochondric” is indicated, in these organelles by integration of a nucleic acid of the invention into the genome of the respective organelle under control of preferable a promoter selective for the respective organelle.

Preferably, said mRNA is the coding region of the nucleic acid molecule of the present invention and/or the protein conferring the increased expression of a protein encoded by the coding region of the nucleic acid molecule of the present invention alone or linked to a transit nucleic acid sequence or transit peptide encoding nucleic acid sequence or the polypeptide having the herein mentioned activity, e.g. conferring the generation of or increase of 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose, respectively, after increasing the expression or activity of the encoded polypeptide, non-targeted or in organelles such as plastids and/or mitochondria, preferably plastids, or having the activity of a polypeptide having an activity as the protein as shown in the respective line in Table II, application no. 17, column 3, or its homologs. Preferably the increase of 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose, respectively, takes place non-targeted or in plastids and/or mitochondria, preferably non-targeted or in plastids.

[0113.1.7.17] to [0122.1.7.17] for the disclosure of this paragraphs see [0113.1.7.7] to [0122.1.7.7] above

The activation of an endogenous polypeptide having above-mentioned activity, e.g. having the activity of a protein as shown in the respective line in Table II, application no. 17, column 3 or of the polypeptide of the invention, e.g. conferring the increase of the fine chemical 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose7-phosphate, sorbitol, sucrose, trehalose, or verbascose, respectively, by increase of expression or activity in the cytoplasm, and/or in the cytosol, and/or in an organelle, such as plastids or mitochondria, can also be increased by introducing a synthetic transcription factor, which binds close to the coding region of the gene encoding the protein as shown in the respective line in Table II, application no. 17, column 5 or 8, or homologs or fragments thereof, and activates its transcription. A chimeric zinc finger protein can be constructed, which comprises a specific DNA-binding domain and an activation domain as e.g. the VP16 domain of Herpes Simplex virus. The specific binding domain can bind to the regulatory region of the gene encoding the protein as shown in the respective line in Table II, application no. 17, column 5 or 8, or homologs or fragments thereof. The expression of the chimeric transcription factor in a non-human organism, in particular in a plant, leads to a specific expression of the protein as shown in the respective line in Table II, application no. 17, column 5 or 8, or homologs or fragments thereof, see e.g. in WO01/52620, Oriz, Proc. Natl. Acad. Sci. USA 99, 13290 (2002), or Guan, Proc. Natl. Acad. Sci. USA 99, 13296 (2002).

[0124.1.7.17] to [0127.1.7.17] for the disclosure of this paragraphs see [0124.1.7.7] to [0127.1.7.7] above

Owing to the introduction of a gene or a plurality of genes conferring the expression of the nucleic acid molecule of the invention or the polypeptide of the invention, for example the nucleic acid construct mentioned below, or encoding the protein as shown in the respective line in Table II, application no. 17, column 5 or 8, or homologs or fragments thereof, into a non-human organism alone or in combination with other genes, it is possible not only to increase the biosynthetic flux towards the end product, but also to increase, modify or create de novo an advantageous, preferably novel metabolites composition in the non-human organism, e.g. an advantageous composition comprising a higher content of (from a viewpoint of nutritional physiology limited) 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose and if desired other saccharides and/or carbohydrates, and/or other metabolites, in free or bound form.

[0129.1.7.17] for the disclosure of this paragraph see [0129.1.7.7] above

Accordingly, in one embodiment, the process according to the invention relates to a process, which comprises:
(a) providing a non-human organism, preferably a microorganism, a plant cell, a plant tissue, a plant or a part thereof, more preferably a microorganism, a plant tissue, a plant or a part thereof;
(b) increasing the activity of a protein as shown in the respective line in Table II, application no. 17, column 3 or of a polypeptide being encoded by the respective nucleic acid molecule of the present invention and described below, e.g. conferring an increase of the fine chemical 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose, respectively, in the non-human organism, preferably in the microorganism, the plant cell, the plant tissue, the plant or a part thereof, more preferably a microorganism, a plant tissue, a plant or a part thereof, especially cytoplasmic or in an organelle, like plastids or mitochondria,
(c) growing the non-human organism, preferably the microorganism, the plant cell, the plant tissue, the plant or a part thereof, under conditions which permit the production of said chemical in the non-human organism, preferably the microorganism, the plant cell, the plant tissue, the plant or a part thereof; and
(d) if desired, recovering, optionally isolating, said fine chemical, in free and/or bound form, and, optionally further free and/or bound saccharides and/or carbohydrates, and/or other metabolites synthesized by the non-human organism, the microorganism, the plant cell, the plant tissue, the plant or a part thereof.

[0131.1.7.17] to [0133.1.7.17] for the disclosure of these paragraphs see [0131.1.7.7] to [0133.1.7.7] above

The non-human organism, in particular the microorganism, plant or part thereof, is advantageously grown in such a way that it is not only possible to recover, if desired to isolate the free or bound fine chemical or the free and bound fine chemical but as option it is also possible to produce, recover and, if desired to isolate, other free or/and bound vitamins and/or other metabolites.

The organisms are harvested in a manner known per se and appropriate for the particular organism. Microorganisms such as bacteria, mosses, yeasts and fungi or plant cells which are cultured in liquid media by fermentation may be removed, for example, by centrifugation, decanting or filtration. Plants are grown on solid media in a manner known per se and harvested accordingly.

The organisms, its cells or a part thereof or the recovered, and if desired isolated, saccharides, means all of the aforementioned carbohydrate, e.g. monosaccharides, preferably fructose and/or glucose, or other pentoses or hexoses; in free or bound form, preferably glucose-1-phosphate and/or sedoheptulose-7-phosphate; and/or derivates of monosaccharides, preferably (sugar) acids, e.g. saccharic acid, 3,4,5-trihydroxypentanoic acid; and/or sugar alcohols e.g. erythrol, galactitol, sorbitol, mannitol and/or myo-inositol; and/or deoxy-derivates, e.g. fucose; and/or disaccharides, preferably sucrose, melibiose, galactinol and/or trehalose, and/or oligosaccharides, preferably the RFOs (raffinose family of oligosaccharides: preferably the trisaccharide raffinose and/or the pentasaccharide verbascose), and/or polysaccharides, preferably starch and/or cellulose and/or oligosaccharides, polysaccharides, salts, amines, ethers, esters, preferably ester of phosphoric acid, glycosides containing said saccharides can then be processed further directly into foodstuffs or animal feeds or for other applications, for example according to the disclosures made in WO/2005/083093 or WO2005/012316, which are expressly incorporated herein by reference. The organism, its cells or a part thereof, such as microorganisms or plants or the recovered, and if desired isolated, respective fine chemical can then be processed further directly into foodstuffs or animal feeds or for other applications in nutrition or medicine or cosmetics, for example according to the disclosures made in U.S. Pat. No. 6,669,962 (Starch microcapsules for delivery of active agents); US No. 20050042737 (Starch process); US 20050054071 (Enzymes for starch processing); US 20050091716 (Novel plants and processes for obtaining them); U.S. Pat. No. 5,096,594 and U.S. Pat. No. 5,482,631 discloses a method of purifying cyclitols; U.S. Pat. No. 4,997,489 discloses soaking almond hulls in water to obtain a syrup containing fructose, glucose, inositol, and sorbitol; U.S. Pat. No. 5,296,364 discloses a microbial method for producing inositol; U.S. Pat. No. 4,734,402; US. 4,788,065; U.S. Pat. No. 6,465,037 and U.S. Pat. No. 6,355,295 relates to soy food ingredient based on carbohydrates, U.S. Pat. No. 6,653,451; US 20040128713: pertains to soybean plants having in their seeds significantly lower contents of raffinose, stachyose and phytic acid and significantly higher contents of sucrose and inorganic phosphate; US 20050008713 discloses compositions of plant carbohydrates for dietary supplements and nutritional support; which are expressly incorporated herein by reference.

[0135.1.7.17] to [0139.1.7.17] for the disclosure of these paragraphs see [0135.1.7.7] to [0139.1.7.7] above

In another embodiment, the invention relates to a (isolated) nucleic acid molecule, which comprises a nucleic acid molecule selected from the group consisting of:
(a) a nucleic acid molecule encoding a polypeptide shown in Table II, application no. 17, column 5 or 8, preferably shown in Table II B, application no. 17, column 8, or a homolog or a fragment thereof;
(b) a nucleic acid molecule shown in Table I, application no. 17, column 5 or 8, preferably shown in Table I B, application no. 17, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 17, preferably in column 8 of Table II B, application no. 17;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 17, preferably shown in column 8 of Table I B, application no. 17, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 17;
(i) a nucleic acid molecule encoding a polypeptide having the activity represented by a respective protein as depicted in column 5 of Table II, application no. 17;
(j) a nucleic acid molecule comprising a polynucleotide which is obtained by amplifying a cDNA library or a genomic library using the primers in Table III, application no. 17, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j); or a nucleic acid molecule comprising a sequence which is complementary thereto.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 17.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II application no. 17 and confers a generation or an increase of the respective fine chemical in a non-human organism or a part thereof as compared to a corresponding wild-type non-human organism or part thereof.

In another embodiment, the invention relates to a (isolated) nucleic acid molecule, which comprises a nucleic acid molecule selected from the group consisting of:
(a) a nucleic acid molecule encoding a polypeptide shown in Table II, column 5 or 8, application no. 17, preferably shown in Table II A, application no. 17, in column 5 or in Table II A, application no. 17, column 8 or in Table II B, application no. 17, column 8, or a homolog or a fragment thereof;
(b) a nucleic acid molecule shown in Table I, application no. 17, column 5 or 8, preferably shown in Table I A, application no. 17, in column 5 or in Table I A, application no. 17, column 8 or in Table I B, application no. 17, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 17, preferably shown in Table II A, application no. 17, in column 5 or in Table II A, application no. 17, column 8 or in Table II B, application no. 17, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 17, preferably shown in Table I A, application no. 17, in column 5 or in Table I A, application no. 17, column 8 or in Table I B column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 17;
(i) a nucleic acid molecule encoding a polypeptide having the activity represented by a respective protein as depicted in column 5 of Table II, application no. 17;
(j) a nucleic acid molecule comprising a polynucleotide which is obtained by amplifying a cDNA library or a genomic library using the primers in Table III, application no. 17, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt,500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto;
   whereby, preferably, the nucleic acid molecule according to (a), (b), (c), (d), (e), (f), (g), (h), (i), (j) or (k) distinguishes over the sequence depicted in Table I A and/or I B, application no. 17, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 17, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 17, column 5 or 8, or the coding region thereof, respectively. In a further embodiment the nucleic acid molecule does not encode the polypeptide sequence shown in Table II A and/or II B, application no. 17, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 17, column 5 or 8 and does not encode a protein of the sequence shown in Table II A and/or II B, application no. 17, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 17, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 17, column 5 or 8 but less than 100%, preferably less than 99.999%, 99.99%, 99.9%, 99%, 98%, 97%, 96% or 95% identical to the sequence shown in Table II A and/or II B, application no. 17, column 5 or 8.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 17.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II, application no. 17, and confers a generation or an increase of the respective fine chemical in a non-human organism or a part thereof as compared to a corresponding wild-type non-human organism or part thereof.

[0144.1.7.17] to [0155.1.7.17] for the disclosure of these paragraphs see [0144.1.7.7] to [0155.1.7.7] above

In a preferred embodiment a nucleic acid construct, for example an expression cassette, comprises upstream, i.e. at the 5′ end of the encoding sequence, a promoter and downstream, i.e. at the 3′ end, a polyadenylation signal and optionally other regulatory elements which are operably linked to the intervening encoding sequence with one of the nucleic acids as depicted in the respective line in Table I, application no. 17, column 5 or 8, preferably the coding region thereof, or a homolog or a fragment thereof. By an operable linkage is meant the sequential arrangement of promoter, encoding sequence, terminator, optionally other regulatory elements and optionally targeting sequences in such a way that each of the regulatory elements/targeting sequence can fulfill its function in the expression of the encoding sequence in due manner. In one embodiment the sequences preferred for operable linkage are targeting sequences for ensuring subcellular localization in organelles, like plastids or mitochondria. However, targeting sequences for ensuring subcellular localization in the endoplasmic reticulum (=ER), in the nucleus, in oil corpuscles or other compartments may also be employed as well as translation promoters such as the 5′ lead sequence in tobacco mosaic virus (Gallie et al., Nucl. Acids Res. 15, 8693 (1987)).

for the disclosure of this paragraph see [0157.1.7.7] above

In an embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 17, column 5 or 8, preferably shown in Table II A, application no. 17, column 5, or in Table II A, application no. 17, column 8, or in Table II B, application no. 17, column 8, or a homolog or a fragment thereof;
   (b) a nucleic acid molecule shown in Table I, application no. 17, column 5 or 8, preferably shown in Table I A, application no. 17, column 5, or in Table I A, application no. 17, column 8, or in Table I B, application no. 17, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 17, preferably shown in Table II A, application no. 17, column 5, or in Table II A, application no. 17, column 8, or in Table II B, application no. 17, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 17, preferably shown in Table I A, application no. 17, column 5, or in Table I A, application no. 17, column 8, or in Table I B, application no. 17, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 17;
   (i) a nucleic acid molecule encoding a polypeptide having the activity represented by a respective protein as depicted in column 5 of Table II, application no. 17;
   (j) a nucleic acid molecule comprising a polynucleotide which is obtained by amplifying a cDNA library or a genomic library using the primers in Table III, application no. 17, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto. and, optionally
3) nucleic acid molecule encoding a transit peptide, preferably encoding a plastidal transit peptide or a mitochondrial transit peptide;
   which are operable linked.
   (All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 17.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 17 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 17, in the same line as of the respective nucleic acid molecule according to 2) “plastidic” is depicted, or encoding a mitochondrial transit peptide, especially in case in column 6 of Table I, application no. 17, in the same line as of the respective nucleic acid molecule according to 2) “mitochondric” is depicted.

In another embodiment thereof the expression cassette does not comprise 3) a nucleic acid molecule encoding a transit peptide, especially in case in column 6 of Table I, application no. 17, in the same line as of the respective nucleic acid molecule according to 2) “non-targeted” is depicted.

In a further embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 17, column 5 or 8, preferably shown in Table II A, application no. 17, column 5, or in Table II A, application no. 17, column 8, or in Table II B, application no. 17, column 8, or a homolog or a fragment thereof;
   (b) a nucleic acid molecule shown in Table I, application no. 17, column 5 or 8, preferably shown in Table I A, application no. 17, column 5, or in Table I A, application no. 17, column 8, or in Table I B, application no. 17, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 17, preferably shown in Table II A, application no. 17, column 5, or in Table II A, application no. 17, column 8, or in Table II B, application no. 17, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 17, preferably shown in Table I A, application no. 17, column 5, or in Table I A, application no. 17, column 8,or in Table I B, application no. 17, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 17;
   (i) a nucleic acid molecule encoding a polypeptide having the activity represented by a respective protein as depicted in column 5 of Table II, application no. 17;
   (j) a nucleic acid molecule comprising a polynucleotide which is obtained by amplifying a cDNA library or a genomic library using the primers in Table III, application no. 17, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, optionally
3) nucleic acid molecule encoding a transit peptide, preferably encoding a plastidal transit peptide or a mitochondrial transit peptide;
   which are operable linked and
   whereby, preferably, the nucleic acid molecule according to 2 (a), (b), (c), (d), (e), (f), (g), (h), (i), (j) or (k) distinguishes over the sequence depicted in Table I A and/or I B, application no. 17, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention according to 2) does not consist of the sequence shown in Table I A and/or I B, application no. 17, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 17, column 5 or 8, or the coding region thereof, respectively. In a further embodiment the nucleic acid molecule does not encode the polypeptide sequence shown in Table II A and/or II B, application no. 17, column 5 or 8 but homologs thereof.

Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 17, column 5 or 8 and does not encode a protein of the sequence shown in Table II A and/or II B, application no. 17, column 5 or 8. Accordingly, in one embodiment, the protein encoded by a sequence of a nucleic acid according to 2(a), (b), (c), (d), (e), (f), (g), (h), (i), (j) or (k) does not consist of the sequence shown in Table I A and/or I B, application no. 17, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 17, column 5 or 8 but less than 100%, 99.999%, 99.99%, 99.9%, 99%, 98%, 97%, 96% or 95% identical to the sequence shown in Table II A and/or II B, application no. 17, column 5 or 8.

(All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 17.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 17 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 17, in the same line as of the respective nucleic acid molecule according to 2) “plastidic” is depicted, or encoding a mitochondrial transit peptide, especially in case in column 6 of Table I, application no. 17, in the same line as of the respective nucleic acid molecule according to 2) “mitochondric” is depicted.

In another embodiment thereof the expression cassette does not comprise 3) a nucleic acid molecule encoding a transit peptide, especially in case in column 6 of Table I, application no. 17, in the same line as of the respective nucleic acid molecule according to 2) “non-targeted” is depicted.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 142992, or a homolog or fragment thereof, or a homolog thereof being depicted in Table I, application no. 17, column 8, in the same line as SEQ ID NO. 142992, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 142992 is depicted, plastidic is mentioned, or a mitochondrial transit peptide in case in Table I, application no. 17, in column 6 in the same line as SEQ ID NO. 142992 is depicted, mitochondric is mentioned; which is operable linked, too.

In another embodiment thereof the expression cassette does not comprise an additional transit peptide in case in Table I, application no. 17, in column 6 in the same line as SEQ ID NO. 142992 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17451, or a homolog or fragment thereof, or a homolog thereof being depicted in Table I, application no. 17, column 8, in the same line as SEQ ID NO. 17451, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 17451 is depicted, plastidic is mentioned, or a mitochondrial transit peptide in case in Table I, application no. 17, in column 6 in the same line as SEQ ID NO. 17451 is depicted, mitochondric is mentioned; which is operable linked, too.

In another embodiment thereof the expression cassette does not comprise an additional transit peptide in case in Table I, application no. 17, in column 6 in the same line as SEQ ID NO. 17451 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 140771, or a homolog or fragment thereof, or a homolog thereof being depicted in Table I, application no. 17, column 8, in the same line as SEQ ID NO. 140771, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 140771 is depicted, plastidic is mentioned, or a mitochondrial transit peptide in case in Table I, application no. 17, in column 6 in the same line as SEQ ID NO. 140771 is depicted, mitochondric is mentioned; which is operable linked, too.

In another embodiment thereof the expression cassette does not comprise an additional transit peptide in case in Table I, application no. 17, in column 6 in the same line as SEQ ID NO. 140771 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 113651, or a homolog or fragment thereof, or a homolog thereof being depicted in Table I, application no. 17, column 8, in the same line as SEQ ID NO. 113651, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 113651 is depicted, plastidic is mentioned, or a mitochondrial transit peptide in case in Table I, application no. 17, in column 6 in the same line as SEQ ID NO. 113651 is depicted, mitochondric is mentioned; which is operable linked, too.

In another embodiment thereof the expression cassette does not comprise an additional transit peptide in case in Table I, application no. 17, in column 6 in the same line as SEQ ID NO. 113651 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17701, or a homolog or fragment thereof, or a homolog thereof being depicted in Table I, application no. 17, column 8, in the same line as SEQ ID NO. 17701, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 17701 is depicted, plastidic is mentioned, or a mitochondrial transit peptide in case in Table I, application no. 17, in column 6 in the same line as SEQ ID NO. 17701 is depicted, mitochondric is mentioned; which is operable linked, too.

In another embodiment thereof the expression cassette does not comprise an additional transit peptide in case in Table I, application no. 17, in column 6 in the same line as SEQ ID NO. 17701 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17968, or a homolog or fragment thereof, or a homolog thereof being depicted in Table I, application no. 17, column 8, in the same line as SEQ ID NO. 17968, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 17968 is depicted, plastidic is mentioned, or a mitochondrial transit peptide in case in Table I, application no. 17, in column 6 in the same line as SEQ ID NO. 17968 is depicted, mitochondric is mentioned; which is operable linked, too.

In another embodiment thereof the expression cassette does not comprise an additional transit peptide in case in Table I, application no. 17, in column 6 in the same line as SEQ ID NO. 17968 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 18070, or a homolog or fragment thereof, or a homolog thereof being depicted in Table I, application no. 17, column 8, in the same line as SEQ ID NO. 18070, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 18070 is depicted, plastidic is mentioned, or a mitochondrial transit peptide in case in Table I, application no. 17, in column 6 in the same line as SEQ ID NO. 18070 is depicted, mitochondric is mentioned; which is operable linked, too.

In another embodiment thereof the expression cassette does not comprise an additional transit peptide in case in Table I, application no. 17, in column 6 in the same line as SEQ ID NO. 18070 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 140841, or a homolog or fragment thereof, or a homolog thereof being depicted in Table I, application no. 17, column 8, in the same line as SEQ ID NO. 140841, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 140841 is depicted, plastidic is mentioned, or a mitochondrial transit peptide in case in Table I, application no. 17, in column 6 in the same line as SEQ ID NO. 140841 is depicted, mitochondric is mentioned; which is operable linked, too.

In another embodiment thereof the expression cassette does not comprise an additional transit peptide in case in Table I, application no. 17, in column 6 in the same line as SEQ ID NO. 140841 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 19419, or a homolog or fragment thereof, or a homolog thereof being depicted in Table I, application no. 17, column 8, in the same line as SEQ ID NO. 19419, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 19419 is depicted, plastidic is mentioned, or a mitochondrial transit peptide in case in Table I, application no. 17, in column 6 in the same line as SEQ ID NO. 19419 is depicted, mitochondric is mentioned; which is operable linked, too.

In another embodiment thereof the expression cassette does not comprise an additional transit peptide in case in Table I, application no. 17, in column 6 in the same line as SEQ ID NO. 19419 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 141013, or a homolog or fragment thereof, or a homolog thereof being depicted in Table I, application no. 17, column 8, in the same line as SEQ ID NO. 141013, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 141013 is depicted, plastidic is mentioned, or a mitochondrial transit peptide in case in Table I, application no. 17, in column 6 in the same line as SEQ ID NO. 141013 is depicted, mitochondric is mentioned; which is operable linked, too.

In another embodiment thereof the expression cassette does not comprise an additional transit peptide in case in Table I, application no. 17, in column 6 in the same line as SEQ ID NO. 141013 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 19502, or a homolog or fragment thereof, or a homolog thereof being depicted in Table I, application no. 17, column 8, in the same line as SEQ ID NO. 19502, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 19502 is depicted, plastidic is mentioned, or a mitochondrial transit peptide in case in Table I, application no. 17, in column 6 in the same line as SEQ ID NO. 19502 is depicted, mitochondric is mentioned; which is operable linked, too.

In another embodiment thereof the expression cassette does not comprise an additional transit peptide in case in Table I, application no. 17, in column 6 in the same line as SEQ ID NO. 19502 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 1298, or a homolog or fragment thereof, or a homolog thereof being depicted in Table I, application no. 17, column 8, in the same line as SEQ ID NO. 1298, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 1298 is depicted, plastidic is mentioned, or a mitochondrial transit peptide in case in Table I, application no. 17, in column 6 in the same line as SEQ ID NO. 1298 is depicted, mitochondric is mentioned; which is operable linked, too.

In another embodiment thereof the expression cassette does not comprise an additional transit peptide in case in Table I, application no. 17, in column 6 in the same line as SEQ ID NO. 1298 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 20346, or a homolog or fragment thereof, or a homolog thereof being depicted in Table I, application no. 17, column 8, in the same line as SEQ ID NO. 20346, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 20346 is depicted, plastidic is mentioned, or a mitochondrial transit peptide in case in Table I, application no. 17, in column 6 in the same line as SEQ ID NO. 20346 is depicted, mitochondric is mentioned; which is operable linked, too.

In another embodiment thereof the expression cassette does not comprise an additional transit peptide in case in Table I, application no. 17, in column 6 in the same line as SEQ ID NO. 20346 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 20578, or a homolog or fragment thereof, or a homolog thereof being depicted in Table I, application no. 17, column 8, in the same line as SEQ ID NO. 20578, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 20578 is depicted, plastidic is mentioned, or a mitochondrial transit peptide in case in Table I, application no. 17, in column 6 in the same line as SEQ ID NO. 20578 is depicted, mitochondric is mentioned; which is operable linked, too.

In another embodiment thereof the expression cassette does not comprise an additional transit peptide in case in Table I, application no. 17, in column 6 in the same line as SEQ ID NO. 20578 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 133042, or a homolog or fragment thereof, or a homolog thereof being depicted in Table I, application no. 17, column 8, in the same line as SEQ ID NO. 133042, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 133042 is depicted, plastidic is mentioned, or a mitochondrial transit peptide in case in Table I, application no. 17, in column 6 in the same line as SEQ ID NO. 133042 is depicted, mitochondric is mentioned; which is operable linked, too.

In another embodiment thereof the expression cassette does not comprise an additional transit peptide in case in Table I, application no. 17, in column 6 in the same line as SEQ ID NO. 133042 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 108727, or a homolog or fragment thereof, or a homolog thereof being depicted in Table I, application no. 17, column 8, in the same line as SEQ ID NO. 108727, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 108727 is depicted, plastidic is mentioned, or a mitochondrial transit peptide in case in Table I, application no. 17, in column 6 in the same line as SEQ ID NO. 108727 is depicted, mitochondric is mentioned; which is operable linked, too.

In another embodiment thereof the expression cassette does not comprise an additional transit peptide in case in Table I, application no. 17, in column 6 in the same line as SEQ ID NO. 108727 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 1623, or a homolog or fragment thereof, or a homolog thereof being depicted in Table I, application no. 17, column 8, in the same line as SEQ ID NO. 1623, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 1623 is depicted, plastidic is mentioned, or a mitochondrial transit peptide in case in Table I, application no. 17, in column 6 in the same line as SEQ ID NO. 1623 is depicted, mitochondric is mentioned; which is operable linked, too.

In another embodiment thereof the expression cassette does not comprise an additional transit peptide in case in Table I, application no. 17, in column 6 in the same line as SEQ ID NO. 1623 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 21106, or a homolog or fragment thereof, or a homolog thereof being depicted in Table I, application no. 17, column 8, in the same line as SEQ ID NO. 21106, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 21106 is depicted, plastidic is mentioned, or a mitochondrial transit peptide in case in Table I, application no. 17, in column 6 in the same line as SEQ ID NO. 21106 is depicted, mitochondric is mentioned; which is operable linked, too.

In another embodiment thereof the expression cassette does not comprise an additional transit peptide in case in Table I, application no. 17, in column 6 in the same line as SEQ ID NO. 21106 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 141637, or a homolog or fragment thereof, or a homolog thereof being depicted in Table I, application no. 17, column 8, in the same line as SEQ ID NO. 141637, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 141637 is depicted, plastidic is mentioned, or a mitochondrial transit peptide in case in Table I, application no. 17, in column 6 in the same line as SEQ ID NO. 141637 is depicted, mitochondric is mentioned; which is operable linked, too.

In another embodiment thereof the expression cassette does not comprise an additional transit peptide in case in Table I, application no. 17, in column 6 in the same line as SEQ ID NO. 141637 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 141637, or a homolog or fragment thereof, or a homolog thereof being depicted in Table I, application no. 17, column 8, in the same line as SEQ ID NO. 141637, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 141637 is depicted, plastidic is mentioned, or a mitochondrial transit peptide in case in Table I, application no. 17, in column 6 in the same line as SEQ ID NO. 141637 is depicted, mitochondric is mentioned; which is operable linked, too.

In another embodiment thereof the expression cassette does not comprise an additional transit peptide in case in Table I, application no. 17, in column 6 in the same line as SEQ ID NO. 141637 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68657, or a homolog or fragment thereof, or a homolog thereof being depicted in Table I, application no. 17, column 8, in the same line as SEQ ID NO. 68657, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 68657 is depicted, plastidic is mentioned, or a mitochondrial transit peptide in case in Table I, application no. 17, in column 6 in the same line as SEQ ID NO. 68657 is depicted, mitochondric is mentioned; which is operable linked, too.

In another embodiment thereof the expression cassette does not comprise an additional transit peptide in case in Table I, application no. 17, in column 6 in the same line as SEQ ID NO. 68657 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 21497, or a homolog or fragment thereof, or a homolog thereof being depicted in Table I, application no. 17, column 8, in the same line as SEQ ID NO. 21497, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 21497 is depicted, plastidic is mentioned, or a mitochondrial transit peptide in case in Table I, application no. 17, in column 6 in the same line as SEQ ID NO. 21497 is depicted, mitochondric is mentioned; which is operable linked, too.

In another embodiment thereof the expression cassette does not comprise an additional transit peptide in case in Table I, application no. 17, in column 6 in the same line as SEQ ID NO. 21497 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 22015, or a homolog or fragment thereof, or a homolog thereof being depicted in Table I, application no. 17, column 8, in the same line as SEQ ID NO. 22015, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 22015 is depicted, plastidic is mentioned, or a mitochondrial transit peptide in case in Table I, application no. 17, in column 6 in the same line as SEQ ID NO. 22015 is depicted, mitochondric is mentioned; which is operable linked, too.

In another embodiment thereof the expression cassette does not comprise an additional transit peptide in case in Table I, application no. 17, in column 6 in the same line as SEQ ID NO. 22015 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 22249, or a homolog or fragment thereof, or a homolog thereof being depicted in Table I, application no. 17, column 8, in the same line as SEQ ID NO. 22249, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 22249 is depicted, plastidic is mentioned, or a mitochondrial transit peptide in case in Table I, application no. 17, in column 6 in the same line as SEQ ID NO. 22249 is depicted, mitochondric is mentioned; which is operable linked, too.

In another embodiment thereof the expression cassette does not comprise an additional transit peptide in case in Table I, application no. 17, in column 6 in the same line as SEQ ID NO. 22249 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 22699, or a homolog or fragment thereof, or a homolog thereof being depicted in Table I, application no. 17, column 8, in the same line as SEQ ID NO. 22699, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 22699 is depicted, plastidic is mentioned, or a mitochondrial transit peptide in case in Table I, application no. 17, in column 6 in the same line as SEQ ID NO. 22699 is depicted, mitochondric is mentioned; which is operable linked, too.

In another embodiment thereof the expression cassette does not comprise an additional transit peptide in case in Table I, application no. 17, in column 6 in the same line as SEQ ID NO. 22699 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 22921, or a homolog or fragment thereof, or a homolog thereof being depicted in Table I, application no. 17, column 8, in the same line as SEQ ID NO. 22921, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 22921 is depicted, plastidic is mentioned, or a mitochondrial transit peptide in case in Table I, application no. 17, in column 6 in the same line as SEQ ID NO. 22921 is depicted, mitochondric is mentioned; which is operable linked, too.

In another embodiment thereof the expression cassette does not comprise an additional transit peptide in case in Table I, application no. 17, in column 6 in the same line as SEQ ID NO. 22921 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 1815, or a homolog or fragment thereof, or a homolog thereof being depicted in Table I, application no. 17, column 8, in the same line as SEQ ID NO. 1815, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 1815 is depicted, plastidic is mentioned, or a mitochondrial transit peptide in case in Table I, application no. 17, in column 6 in the same line as SEQ ID NO. 1815 is depicted, mitochondric is mentioned; which is operable linked, too.

In another embodiment thereof the expression cassette does not comprise an additional transit peptide in case in Table I, application no. 17, in column 6 in the same line as SEQ ID NO. 1815 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68727, or a homolog or fragment thereof, or a homolog thereof being depicted in Table I, application no. 17, column 8, in the same line as SEQ ID NO. 68727, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 68727 is depicted, plastidic is mentioned, or a mitochondrial transit peptide in case in Table I, application no. 17, in column 6 in the same line as SEQ ID NO. 68727 is depicted, mitochondric is mentioned; which is operable linked, too.

In another embodiment thereof the expression cassette does not comprise an additional transit peptide in case in Table I, application no. 17, in column 6 in the same line as SEQ ID NO. 68727 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 141758, or a homolog or fragment thereof, or a homolog thereof being depicted in Table I, application no. 17, column 8, in the same line as SEQ ID NO. 141758, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 141758 is depicted, plastidic is mentioned, or a mitochondrial transit peptide in case in Table I, application no. 17, in column 6 in the same line as SEQ ID NO. 141758 is depicted, mitochondric is mentioned; which is operable linked, too.

In another embodiment thereof the expression cassette does not comprise an additional transit peptide in case in Table I, application no. 17, in column 6 in the same line as SEQ ID NO. 141758 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 2367, or a homolog or fragment thereof, or a homolog thereof being depicted in Table I, application no. 17, column 8, in the same line as SEQ ID NO. 2367, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 2367 is depicted, plastidic is mentioned, or a mitochondrial transit peptide in case in Table I, application no. 17, in column 6 in the same line as SEQ ID NO. 2367 is depicted, mitochondric is mentioned; which is operable linked, too.

In another embodiment thereof the expression cassette does not comprise an additional transit peptide in case in Table I, application no. 17, in column 6 in the same line as SEQ ID NO. 2367 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 2573, or a homolog or fragment thereof, or a homolog thereof being depicted in Table I, application no. 17, column 8, in the same line as SEQ ID NO. 2573, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 2573 is depicted, plastidic is mentioned, or a mitochondrial transit peptide in case in Table I, application no. 17, in column 6 in the same line as SEQ ID NO. 2573 is depicted, mitochondric is mentioned; which is operable linked, too.

In another embodiment thereof the expression cassette does not comprise an additional transit peptide in case in Table I, application no. 17, in column 6 in the same line as SEQ ID NO. 2573 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 2935, or a homolog or fragment thereof, or a homolog thereof being depicted in Table I, application no. 17, column 8, in the same line as SEQ ID NO. 2935, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 2935 is depicted, plastidic is mentioned, or a mitochondrial transit peptide in case in Table I, application no. 17, in column 6 in the same line as SEQ ID NO. 2935 is depicted, mitochondric is mentioned; which is operable linked, too.

In another embodiment thereof the expression cassette does not comprise an additional transit peptide in case in Table I, application no. 17, in column 6 in the same line as SEQ ID NO. 2935 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 109717, or a homolog or fragment thereof, or a homolog thereof being depicted in Table I, application no. 17, column 8, in the same line as SEQ ID NO. 109717, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 109717 is depicted, plastidic is mentioned, or a mitochondrial transit peptide in case in Table I, application no. 17, in column 6 in the same line as SEQ ID NO. 109717 is depicted, mitochondric is mentioned; which is operable linked, too.

In another embodiment thereof the expression cassette does not comprise an additional transit peptide in case in Table I, application no. 17, in column 6 in the same line as SEQ ID NO. 109717 is depicted, non-targeted is mentioned. In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 23482, or a homolog or fragment thereof, or a homolog thereof being depicted in Table I, application no. 17, column 8, in the same line as SEQ ID NO. 23482, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 23482 is depicted, plastidic is mentioned, or a mitochondrial transit peptide in case in Table I, application no. 17, in column 6 in the same line as SEQ ID NO. 23482 is depicted, mitochondric is mentioned; which is operable linked, too.

In another embodiment thereof the expression cassette does not comprise an additional transit peptide in case in Table I, application no. 17, in column 6 in the same line as SEQ ID NO. 23482 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 3279, or a homolog or fragment thereof, or a homolog thereof being depicted in Table I, application no. 17, column 8, in the same line as SEQ ID NO. 3279, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 3279 is depicted, plastidic is mentioned, or a mitochondrial transit peptide in case in Table I, application no. 17, in column 6 in the same line as SEQ ID NO. 3279 is depicted, mitochondric is mentioned; which is operable linked, too.

In another embodiment thereof the expression cassette does not comprise an additional transit peptide in case in Table I, application no. 17, in column 6 in the same line as SEQ ID NO. 3279 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 23844, or a homolog or fragment thereof, or a homolog thereof being depicted in Table I, application no. 17, column 8, in the same line as SEQ ID NO. 23844, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 23844 is depicted, plastidic is mentioned, or a mitochondrial transit peptide in case in Table I, application no. 17, in column 6 in the same line as SEQ ID NO. 23844 is depicted, mitochondric is mentioned; which is operable linked, too.

In another embodiment thereof the expression cassette does not comprise an additional transit peptide in case in Table I, application no. 17, in column 6 in the same line as SEQ ID NO. 23844 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 3654, or a homolog or fragment thereof, or a homolog thereof being depicted in Table I, application no. 17, column 8, in the same line as SEQ ID NO. 3654, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 3654 is depicted, plastidic is mentioned, or a mitochondrial transit peptide in case in Table I, application no. 17, in column 6 in the same line as SEQ ID NO. 3654 is depicted, mitochondric is mentioned; which is operable linked, too.

In another embodiment thereof the expression cassette does not comprise an additional transit peptide in case in Table I, application no. 17, in column 6 in the same line as SEQ ID NO. 3654 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68849, or a homolog or fragment thereof, or a homolog thereof being depicted in Table I, application no. 17, column 8, in the same line as SEQ ID NO. 68849, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 68849 is depicted, plastidic is mentioned, or a mitochondrial transit peptide in case in Table I, application no. 17, in column 6 in the same line as SEQ ID NO. 68849 is depicted, mitochondric is mentioned; which is operable linked, too.

In another embodiment thereof the expression cassette does not comprise an additional transit peptide in case in Table I, application no. 17, in column 6 in the same line as SEQ ID NO. 68849 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4102, or a homolog or fragment thereof, or a homolog thereof being depicted in Table I, application no. 17, column 8, in the same line as SEQ ID NO. 4102, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 4102 is depicted, plastidic is mentioned, or a mitochondrial transit peptide in case in Table I, application no. 17, in column 6 in the same line as SEQ ID NO. 4102 is depicted, mitochondric is mentioned; which is operable linked, too.

In another embodiment thereof the expression cassette does not comprise an additional transit peptide in case in Table I, application no. 17, in column 6 in the same line as SEQ ID NO. 4102 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 24311, or a homolog or fragment thereof, or a homolog thereof being depicted in Table I, application no. 17, column 8, in the same line as SEQ ID NO. 24311, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 24311 is depicted, plastidic is mentioned, or a mitochondrial transit peptide in case in Table I, application no. 17, in column 6 in the same line as SEQ ID NO. 24311 is depicted, mitochondric is mentioned; which is operable linked, too.

In another embodiment thereof the expression cassette does not comprise an additional transit peptide in case in Table I, application no. 17, in column 6 in the same line as SEQ ID NO. 24311 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4348, or a homolog or fragment thereof, or a homolog thereof being depicted in Table I, application no. 17, column 8, in the same line as SEQ ID NO. 4348, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 4348 is depicted, plastidic is mentioned, or a mitochondrial transit peptide in case in Table I, application no. 17, in column 6 in the same line as SEQ ID NO. 4348 is depicted, mitochondric is mentioned; which is operable linked, too.

In another embodiment thereof the expression cassette does not comprise an additional transit peptide in case in Table I, application no. 17, in column 6 in the same line as SEQ ID NO. 4348 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68987, or a homolog or fragment thereof, or a homolog thereof being depicted in Table I, application no. 17, column 8, in the same line as SEQ ID NO. 68987, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 68987 is depicted, plastidic is mentioned, or a mitochondrial transit peptide in case in Table I, application no. 17, in column 6 in the same line as SEQ ID NO. 68987 is depicted, mitochondric is mentioned; which is operable linked, too.

In another embodiment thereof the expression cassette does not comprise an additional transit peptide in case in Table I, application no. 17, in column 6 in the same line as SEQ ID NO. 68987 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 24438, or a homolog or fragment thereof, or a homolog thereof being depicted in Table I, application no. 17, column 8, in the same line as SEQ ID NO. 24438, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 24438 is depicted, plastidic is mentioned, or a mitochondrial transit peptide in case in Table I, application no. 17, in column 6 in the same line as SEQ ID NO. 24438 is depicted, mitochondric is mentioned; which is operable linked, too.

In another embodiment thereof the expression cassette does not comprise an additional transit peptide in case in Table I, application no. 17, in column 6 in the same line as SEQ ID NO. 24438 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 24492, or a homolog or fragment thereof, or a homolog thereof being depicted in Table I, application no. 17, column 8, in the same line as SEQ ID NO. 24492, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 24492 is depicted, plastidic is mentioned, or a mitochondrial transit peptide in case in Table I, application no. 17, in column 6 in the same line as SEQ ID NO. 24492 is depicted, mitochondric is mentioned; which is operable linked, too.

In another embodiment thereof the expression cassette does not comprise an additional transit peptide in case in Table I, application no. 17, in column 6 in the same line as SEQ ID NO. 24492 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 142753, or a homolog or fragment thereof, or a homolog thereof being depicted in Table I, application no. 17, column 8, in the same line as SEQ ID NO. 142753, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 142753 is depicted, plastidic is mentioned, or a mitochondrial transit peptide in case in Table I, application no. 17, in column 6 in the same line as SEQ ID NO. 142753 is depicted, mitochondric is mentioned; which is operable linked, too.

In another embodiment thereof the expression cassette does not comprise an additional transit peptide in case in Table I, application no. 17, in column 6 in the same line as SEQ ID NO. 142753 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 69275, or a homolog or fragment thereof, or a homolog thereof being depicted in Table I, application no. 17, column 8, in the same line as SEQ ID NO. 69275, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 69275 is depicted, plastidic is mentioned, or a mitochondrial transit peptide in case in Table I, application no. 17, in column 6 in the same line as SEQ ID NO. 69275 is depicted, mitochondric is mentioned; which is operable linked, too.

In another embodiment thereof the expression cassette does not comprise an additional transit peptide in case in Table I, application no. 17, in column 6 in the same line as SEQ ID NO. 69275 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 25283, or a homolog or fragment thereof, or a homolog thereof being depicted in Table I, application no. 17, column 8, in the same line as SEQ ID NO. 25283, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 25283 is depicted, plastidic is mentioned, or a mitochondrial transit peptide in case in Table I, application no. 17, in column 6 in the same line as SEQ ID NO. 25283 is depicted, mitochondric is mentioned; which is operable linked, too.

In another embodiment thereof the expression cassette does not comprise an additional transit peptide in case in Table I, application no. 17, in column 6 in the same line as SEQ ID NO. 25283 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4904, or a homolog or fragment thereof, or a homolog thereof being depicted in Table I, application no. 17, column 8, in the same line as SEQ ID NO. 4904, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 4904 is depicted, plastidic is mentioned, or a mitochondrial transit peptide in case in Table I, application no. 17, in column 6 in the same line as SEQ ID NO. 4904 is depicted, mitochondric is mentioned; which is operable linked, too.

In another embodiment thereof the expression cassette does not comprise an additional transit peptide in case in Table I, application no. 17, in column 6 in the same line as SEQ ID NO. 4904 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 25428, or a homolog or fragment thereof, or a homolog thereof being depicted in Table I, application no. 17, column 8, in the same line as SEQ ID NO. 25428, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 25428 is depicted, plastidic is mentioned, or a mitochondrial transit peptide in case in Table I, application no. 17, in column 6 in the same line as SEQ ID NO. 25428 is depicted, mitochondric is mentioned; which is operable linked, too.

In another embodiment thereof the expression cassette does not comprise an additional transit peptide in case in Table I, application no. 17, in column 6 in the same line as SEQ ID NO. 25428 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 142929, or a homolog or fragment thereof, or a homolog thereof being depicted in Table I, application no. 17, column 8, in the same line as SEQ ID NO. 142929, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 142929 is depicted, plastidic is mentioned, or a mitochondrial transit peptide in case in Table I, application no. 17, in column 6 in the same line as SEQ ID NO. 142929 is depicted, mitochondric is mentioned; which is operable linked, too.

In another embodiment thereof the expression cassette does not comprise an additional transit peptide in case in Table I, application no. 17, in column 6 in the same line as SEQ ID NO. 142929 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 142933, or a homolog or fragment thereof, or a homolog thereof being depicted in Table I, application no. 17, column 8, in the same line as SEQ ID NO. 142933, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 142933 is depicted, plastidic is mentioned, or a mitochondrial transit peptide in case in Table I, application no. 17, in column 6 in the same line as SEQ ID NO. 142933 is depicted, mitochondric is mentioned; which is operable linked, too.

In another embodiment thereof the expression cassette does not comprise an additional transit peptide in case in Table I, application no. 17, in column 6 in the same line as SEQ ID NO. 142933 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 5318, or a homolog or fragment thereof, or a homolog thereof being depicted in Table I, application no. 17, column 8, in the same line as SEQ ID NO. 5318, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 5318 is depicted, plastidic is mentioned, or a mitochondrial transit peptide in case in Table I, application no. 17, in column 6 in the same line as SEQ ID NO. 5318 is depicted, mitochondric is mentioned; which is operable linked, too.

In another embodiment thereof the expression cassette does not comprise an additional transit peptide in case in Table I, application no. 17, in column 6 in the same line as SEQ ID NO. 5318 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 70006, or a homolog or fragment thereof, or a homolog thereof being depicted in Table I, application no. 17, column 8, in the same line as SEQ ID NO. 70006, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 70006 is depicted, plastidic is mentioned, or a mitochondrial transit peptide in case in Table I, application no. 17, in column 6 in the same line as SEQ ID NO. 70006 is depicted, mitochondric is mentioned; which is operable linked, too.

In another embodiment thereof the expression cassette does not comprise an additional transit peptide in case in Table I, application no. 17, in column 6 in the same line as SEQ ID NO. 70006 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 102899, or a homolog or fragment thereof, or a homolog thereof being depicted in Table I, application no. 17, column 8, in the same line as SEQ ID NO. 102899, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 102899 is depicted, plastidic is mentioned, or a mitochondrial transit peptide in case in Table I, application no. 17, in column 6 in the same line as SEQ ID NO. 102899 is depicted, mitochondric is mentioned; which is operable linked, too.

In another embodiment thereof the expression cassette does not comprise an additional transit peptide in case in Table I, application no. 17, in column 6 in the same line as SEQ ID NO. 102899 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 122803, or a homolog or fragment thereof, or a homolog thereof being depicted in Table I, application no. 17, column 8, in the same line as SEQ ID NO. 122803, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 122803 is depicted, plastidic is mentioned, or a mitochondrial transit peptide in case in Table I, application no. 17, in column 6 in the same line as SEQ ID NO. 122803 is depicted, mitochondric is mentioned; which is operable linked, too.

In another embodiment thereof the expression cassette does not comprise an additional transit peptide in case in Table I, application no. 17, in column 6 in the same line as SEQ ID NO. 122803 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 26120, or a homolog or fragment thereof, or a homolog thereof being depicted in Table I, application no. 17, column 8, in the same line as SEQ ID NO. 26120, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 26120 is depicted, plastidic is mentioned, or a mitochondrial transit peptide in case in Table I, application no. 17, in column 6 in the same line as SEQ ID NO. 26120 is depicted, mitochondric is mentioned; which is operable linked, too.

In another embodiment thereof the expression cassette does not comprise an additional transit peptide in case in Table I, application no. 17, in column 6 in the same line as SEQ ID NO. 26120 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 26196, or a homolog or fragment thereof, or a homolog thereof being depicted in Table I, application no. 17, column 8, in the same line as SEQ ID NO. 26196, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 26196 is depicted, plastidic is mentioned, or a mitochondrial transit peptide in case in Table I, application no. 17, in column 6 in the same line as SEQ ID NO. 26196 is depicted, mitochondric is mentioned; which is operable linked, too.

In another embodiment thereof the expression cassette does not comprise an additional transit peptide in case in Table I, application no. 17, in column 6 in the same line as SEQ ID NO. 26196 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6040, or a homolog or fragment thereof, or a homolog thereof being depicted in Table I, application no. 17, column 8, in the same line as SEQ ID NO. 6040, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 6040 is depicted, plastidic is mentioned, or a mitochondrial transit peptide in case in Table I, application no. 17, in column 6 in the same line as SEQ ID NO. 6040 is depicted, mitochondric is mentioned; which is operable linked, too.

In another embodiment thereof the expression cassette does not comprise an additional transit peptide in case in Table I, application no. 17, in column 6 in the same line as SEQ ID NO. 6040 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 27021, or a homolog or fragment thereof, or a homolog thereof being depicted in Table I, application no. 17, column 8, in the same line as SEQ ID NO. 27021, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 27021 is depicted, plastidic is mentioned, or a mitochondrial transit peptide in case in Table I, application no. 17, in column 6 in the same line as SEQ ID NO. 27021 is depicted, mitochondric is mentioned; which is operable linked, too.

In another embodiment thereof the expression cassette does not comprise an additional transit peptide in case in Table I, application no. 17, in column 6 in the same line as SEQ ID NO. 27021 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 27882, or a homolog or fragment thereof, or a homolog thereof being depicted in Table I, application no. 17, column 8, in the same line as SEQ ID NO. 27882, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 27882 is depicted, plastidic is mentioned, or a mitochondrial transit peptide in case in Table I, application no. 17, in column 6 in the same line as SEQ ID NO. 27882 is depicted, mitochondric is mentioned; which is operable linked, too.

In another embodiment thereof the expression cassette does not comprise an additional transit peptide in case in Table I, application no. 17, in column 6 in the same line as SEQ ID NO. 27882 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 28040, or a homolog or fragment thereof, or a homolog thereof being depicted in Table I, application no. 17, column 8, in the same line as SEQ ID NO. 28040, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 28040 is depicted, plastidic is mentioned, or a mitochondrial transit peptide in case in Table I, application no. 17, in column 6 in the same line as SEQ ID NO. 28040 is depicted, mitochondric is mentioned; which is operable linked, too.

In another embodiment thereof the expression cassette does not comprise an additional transit peptide in case in Table I, application no. 17, in column 6 in the same line as SEQ ID NO. 28040 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6075, or a homolog or fragment thereof, or a homolog thereof being depicted in Table I, application no. 17, column 8, in the same line as SEQ ID NO. 6075, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 6075 is depicted, plastidic is mentioned, or a mitochondrial transit peptide in case in Table I, application no. 17, in column 6 in the same line as SEQ ID NO. 6075 is depicted, mitochondric is mentioned; which is operable linked, too.

In another embodiment thereof the expression cassette does not comprise an additional transit peptide in case in Table I, application no. 17, in column 6 in the same line as SEQ ID NO. 6075 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 72346, or a homolog or fragment thereof, or a homolog thereof being depicted in Table I, application no. 17, column 8, in the same line as SEQ ID NO. 72346, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 72346 is depicted, plastidic is mentioned, or a mitochondrial transit peptide in case in Table I, application no. 17, in column 6 in the same line as SEQ ID NO. 72346 is depicted, mitochondric is mentioned; which is operable linked, too.

In another embodiment thereof the expression cassette does not comprise an additional transit peptide in case in Table I, application no. 17, in column 6 in the same line as SEQ ID NO. 72346 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 29286, or a homolog or fragment thereof, or a homolog thereof being depicted in Table I, application no. 17, column 8, in the same line as SEQ ID NO. 29286, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 29286 is depicted, plastidic is mentioned, or a mitochondrial transit peptide in case in Table I, application no. 17, in column 6 in the same line as SEQ ID NO. 29286 is depicted, mitochondric is mentioned; which is operable linked, too.

In another embodiment thereof the expression cassette does not comprise an additional transit peptide in case in Table I, application no. 17, in column 6 in the same line as SEQ ID NO. 29286 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 29500, or a homolog or fragment thereof, or a homolog thereof being depicted in Table I, application no. 17, column 8, in the same line as SEQ ID NO. 29500, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 29500 is depicted, plastidic is mentioned, or a mitochondrial transit peptide in case in Table I, application no. 17, in column 6 in the same line as SEQ ID NO. 29500 is depicted, mitochondric is mentioned; which is operable linked, too.

In another embodiment thereof the expression cassette does not comprise an additional transit peptide in case in Table I, application no. 17, in column 6 in the same line as SEQ ID NO. 29500 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 111155, or a homolog or fragment thereof, or a homolog thereof being depicted in Table I, application no. 17, column 8, in the same line as SEQ ID NO. 111155, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 111155 is depicted, plastidic is mentioned, or a mitochondrial transit peptide in case in Table I, application no. 17, in column 6 in the same line as SEQ ID NO. 111155 is depicted, mitochondric is mentioned; which is operable linked, too.

In another embodiment thereof the expression cassette does not comprise an additional transit peptide in case in Table I, application no. 17, in column 6 in the same line as SEQ ID NO. 111155 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 31026, or a homolog or fragment thereof, or a homolog thereof being depicted in Table I, application no. 17, column 8, in the same line as SEQ ID NO. 31026, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 31026 is depicted, plastidic is mentioned, or a mitochondrial transit peptide in case in Table I, application no. 17, in column 6 in the same line as SEQ ID NO. 31026 is depicted, mitochondric is mentioned; which is operable linked, too.

In another embodiment thereof the expression cassette does not comprise an additional transit peptide in case in Table I, application no. 17, in column 6 in the same line as SEQ ID NO. 31026 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 143121, or a homolog or fragment thereof, or a homolog thereof being depicted in Table I, application no. 17, column 8, in the same line as SEQ ID NO. 143121, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 143121 is depicted, plastidic is mentioned, or a mitochondrial transit peptide in case in Table I, application no. 17, in column 6 in the same line as SEQ ID NO. 143121 is depicted, mitochondric is mentioned; which is operable linked, too.

In another embodiment thereof the expression cassette does not comprise an additional transit peptide in case in Table I, application no. 17, in column 6 in the same line as SEQ ID NO. 143121 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 114231, or a homolog or fragment thereof, or a homolog thereof being depicted in Table I, application no. 17, column 8, in the same line as SEQ ID NO. 114231, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 114231 is depicted, plastidic is mentioned, or a mitochondrial transit peptide in case in Table I, application no. 17, in column 6 in the same line as SEQ ID NO. 114231 is depicted, mitochondric is mentioned; which is operable linked, too.

In another embodiment thereof the expression cassette does not comprise an additional transit peptide in case in Table I, application no. 17, in column 6 in the same line as SEQ ID NO. 114231 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 143398, or a homolog or fragment thereof, or a homolog thereof being depicted in Table I, application no. 17, column 8, in the same line as SEQ ID NO. 143398, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 143398 is depicted, plastidic is mentioned, or a mitochondrial transit peptide in case in Table I, application no. 17, in column 6 in the same line as SEQ ID NO. 143398 is depicted, mitochondric is mentioned; which is operable linked, too.

In another embodiment thereof the expression cassette does not comprise an additional transit peptide in case in Table I, application no. 17, in column 6 in the same line as SEQ ID NO. 143398 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 73038, or a homolog or fragment thereof, or a homolog thereof being depicted in Table I, application no. 17, column 8, in the same line as SEQ ID NO. 73038, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 73038 is depicted, plastidic is mentioned, or a mitochondrial transit peptide in case in Table I, application no. 17, in column 6 in the same line as SEQ ID NO. 73038 is depicted, mitochondric is mentioned; which is operable linked, too.

In another embodiment thereof the expression cassette does not comprise an additional transit peptide in case in Table I, application no. 17, in column 6 in the same line as SEQ ID NO. 73038 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 73719, or a homolog or fragment thereof, or a homolog thereof being depicted in Table I, application no. 17, column 8, in the same line as SEQ ID NO. 73719, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 73719 is depicted, plastidic is mentioned, or a mitochondrial transit peptide in case in Table I, application no. 17, in column 6 in the same line as SEQ ID NO. 73719 is depicted, mitochondric is mentioned; which is operable linked, too.

In another embodiment thereof the expression cassette does not comprise an additional transit peptide in case in Table I, application no. 17, in column 6 in the same line as SEQ ID NO. 73719 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 102941, or a homolog or fragment thereof, or a homolog thereof being depicted in Table I, application no. 17, column 8, in the same line as SEQ ID NO. 102941, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 102941 is depicted, plastidic is mentioned, or a mitochondrial transit peptide in case in Table I, application no. 17, in column 6 in the same line as SEQ ID NO. 102941 is depicted, mitochondric is mentioned; which is operable linked, too.

In another embodiment thereof the expression cassette does not comprise an additional transit peptide in case in Table I, application no. 17, in column 6 in the same line as SEQ ID NO. 102941 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6510, or a homolog or fragment thereof, or a homolog thereof being depicted in Table I, application no. 17, column 8, in the same line as SEQ ID NO. 6510, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 6510 is depicted, plastidic is mentioned, or a mitochondrial transit peptide in case in Table I, application no. 17, in column 6 in the same line as SEQ ID NO. 6510 is depicted, mitochondric is mentioned; which is operable linked, too.

In another embodiment thereof the expression cassette does not comprise an additional transit peptide in case in Table I, application no. 17, in column 6 in the same line as SEQ ID NO. 6510 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 32037, or a homolog or fragment thereof, or a homolog thereof being depicted in Table I, application no. 17, column 8, in the same line as SEQ ID NO. 32037, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 32037 is depicted, plastidic is mentioned, or a mitochondrial transit peptide in case in Table I, application no. 17, in column 6 in the same line as SEQ ID NO. 32037 is depicted, mitochondric is mentioned; which is operable linked, too.

In another embodiment thereof the expression cassette does not comprise an additional transit peptide in case in Table I, application no. 17, in column 6 in the same line as SEQ ID NO. 32037 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 32308, or a homolog or fragment thereof, or a homolog thereof being depicted in Table I, application no. 17, column 8, in the same line as SEQ ID NO. 32308, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 32308 is depicted, plastidic is mentioned, or a mitochondrial transit peptide in case in Table I, application no. 17, in column 6 in the same line as SEQ ID NO. 32308 is depicted, mitochondric is mentioned; which is operable linked, too.

In another embodiment thereof the expression cassette does not comprise an additional transit peptide in case in Table I, application no. 17, in column 6 in the same line as SEQ ID NO. 32308 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 143495, or a homolog or fragment thereof, or a homolog thereof being depicted in Table I, application no. 17, column 8, in the same line as SEQ ID NO. 143495, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 143495 is depicted, plastidic is mentioned, or a mitochondrial transit peptide in case in Table I, application no. 17, in column 6 in the same line as SEQ ID NO. 143495 is depicted, mitochondric is mentioned; which is operable linked, too.

In another embodiment thereof the expression cassette does not comprise an additional transit peptide in case in Table I, application no. 17, in column 6 in the same line as SEQ ID NO. 143495 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6674, or a homolog or fragment thereof, or a homolog thereof being depicted in Table I, application no. 17, column 8, in the same line as SEQ ID NO. 6674, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 6674 is depicted, plastidic is mentioned, or a mitochondrial transit peptide in case in Table I, application no. 17, in column 6 in the same line as SEQ ID NO. 6674 is depicted, mitochondric is mentioned; which is operable linked, too.

In another embodiment thereof the expression cassette does not comprise an additional transit peptide in case in Table I, application no. 17, in column 6 in the same line as SEQ ID NO. 6674 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 143614, or a homolog or fragment thereof, or a homolog thereof being depicted in Table I, application no. 17, column 8, in the same line as SEQ ID NO. 143614, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 143614 is depicted, plastidic is mentioned, or a mitochondrial transit peptide in case in Table I, application no. 17, in column 6 in the same line as SEQ ID NO. 143614 is depicted, mitochondric is mentioned; which is operable linked, too.

In another embodiment thereof the expression cassette does not comprise an additional transit peptide in case in Table I, application no. 17, in column 6 in the same line as SEQ ID NO. 143614 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 74729, or a homolog or fragment thereof, or a homolog thereof being depicted in Table I, application no. 17, column 8, in the same line as SEQ ID NO. 74729, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 74729 is depicted, plastidic is mentioned, or a mitochondrial transit peptide in case in Table I, application no. 17, in column 6 in the same line as SEQ ID NO. 74729 is depicted, mitochondric is mentioned; which is operable linked, too.

In another embodiment thereof the expression cassette does not comprise an additional transit peptide in case in Table I, application no. 17, in column 6 in the same line as SEQ ID NO. 74729 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 34044, or a homolog or fragment thereof, or a homolog thereof being depicted in Table I, application no. 17, column 8, in the same line as SEQ ID NO. 34044, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 34044 is depicted, plastidic is mentioned, or a mitochondrial transit peptide in case in Table I, application no. 17, in column 6 in the same line as SEQ ID NO. 34044 is depicted, mitochondric is mentioned; which is operable linked, too.

In another embodiment thereof the expression cassette does not comprise an additional transit peptide in case in Table I, application no. 17, in column 6 in the same line as SEQ ID NO. 34044 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 34204, or a homolog or fragment thereof, or a homolog thereof being depicted in Table I, application no. 17, column 8, in the same line as SEQ ID NO. 34204, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 34204 is depicted, plastidic is mentioned, or a mitochondrial transit peptide in case in Table I, application no. 17, in column 6 in the same line as SEQ ID NO. 34204 is depicted, mitochondric is mentioned; which is operable linked, too.

In another embodiment thereof the expression cassette does not comprise an additional transit peptide in case in Table I, application no. 17, in column 6 in the same line as SEQ ID NO. 34204 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 34301, or a homolog or fragment thereof, or a homolog thereof being depicted in Table I, application no. 17, column 8, in the same line as SEQ ID NO. 34301, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 34301 is depicted, plastidic is mentioned, or a mitochondrial transit peptide in case in Table I, application no. 17, in column 6 in the same line as SEQ ID NO. 34301 is depicted, mitochondric is mentioned; which is operable linked, too.

In another embodiment thereof the expression cassette does not comprise an additional transit peptide in case in Table I, application no. 17, in column 6 in the same line as SEQ ID NO. 34301 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 34602, or a homolog or fragment thereof, or a homolog thereof being depicted in Table I, application no. 17, column 8, in the same line as SEQ ID NO. 34602, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 34602 is depicted, plastidic is mentioned, or a mitochondrial transit peptide in case in Table I, application no. 17, in column 6 in the same line as SEQ ID NO. 34602 is depicted, mitochondric is mentioned; which is operable linked, too.

In another embodiment thereof the expression cassette does not comprise an additional transit peptide in case in Table I, application no. 17, in column 6 in the same line as SEQ ID NO. 34602 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 103433, or a homolog or fragment thereof, or a homolog thereof being depicted in Table I, application no. 17, column 8, in the same line as SEQ ID NO. 103433, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 103433 is depicted, plastidic is mentioned, or a mitochondrial transit peptide in case in Table I, application no. 17, in column 6 in the same line as SEQ ID NO. 103433 is depicted, mitochondric is mentioned; which is operable linked, too.

In another embodiment thereof the expression cassette does not comprise an additional transit peptide in case in Table I, application no. 17, in column 6 in the same line as SEQ ID NO. 103433 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 34889, or a homolog or fragment thereof, or a homolog thereof being depicted in Table I, application no. 17, column 8, in the same line as SEQ ID NO. 34889, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 34889 is depicted, plastidic is mentioned, or a mitochondrial transit peptide in case in Table I, application no. 17, in column 6 in the same line as SEQ ID NO. 34889 is depicted, mitochondric is mentioned; which is operable linked, too.

In another embodiment thereof the expression cassette does not comprise an additional transit peptide in case in Table I, application no. 17, in column 6 in the same line as SEQ ID NO. 34889 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 103958, or a homolog or fragment thereof, or a homolog thereof being depicted in Table I, application no. 17, column 8, in the same line as SEQ ID NO. 103958, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 103958 is depicted, plastidic is mentioned, or a mitochondrial transit peptide in case in Table I, application no. 17, in column 6 in the same line as SEQ ID NO. 103958 is depicted, mitochondric is mentioned; which is operable linked, too.

In another embodiment thereof the expression cassette does not comprise an additional transit peptide in case in Table I, application no. 17, in column 6 in the same line as SEQ ID NO. 103958 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 35204, or a homolog or fragment thereof, or a homolog thereof being depicted in Table I, application no. 17, column 8, in the same line as SEQ ID NO. 35204, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 35204 is depicted, plastidic is mentioned, or a mitochondrial transit peptide in case in Table I, application no. 17, in column 6 in the same line as SEQ ID NO. 35204 is depicted, mitochondric is mentioned; which is operable linked, too.

In another embodiment thereof the expression cassette does not comprise an additional transit peptide in case in Table I, application no. 17, in column 6 in the same line as SEQ ID NO. 35204 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 35482, or a homolog or fragment thereof, or a homolog thereof being depicted in Table I, application no. 17, column 8, in the same line as SEQ ID NO. 35482, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 35482 is depicted, plastidic is mentioned, or a mitochondrial transit peptide in case in Table I, application no. 17, in column 6 in the same line as SEQ ID NO. 35482 is depicted, mitochondric is mentioned; which is operable linked, too.

In another embodiment thereof the expression cassette does not comprise an additional transit peptide in case in Table I, application no. 17, in column 6 in the same line as SEQ ID NO. 35482 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7081, or a homolog or fragment thereof, or a homolog thereof being depicted in Table I, application no. 17, column 8, in the same line as SEQ ID NO. 7081, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 7081 is depicted, plastidic is mentioned, or a mitochondrial transit peptide in case in Table I, application no. 17, in column 6 in the same line as SEQ ID NO. 7081 is depicted, mitochondric is mentioned; which is operable linked, too.

In another embodiment thereof the expression cassette does not comprise an additional transit peptide in case in Table I, application no. 17, in column 6 in the same line as SEQ ID NO. 7081 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 35590, or a homolog or fragment thereof, or a homolog thereof being depicted in Table I, application no. 17, column 8, in the same line as SEQ ID NO. 35590, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 35590 is depicted, plastidic is mentioned, or a mitochondrial transit peptide in case in Table I, application no. 17, in column 6 in the same line as SEQ ID NO. 35590 is depicted, mitochondric is mentioned; which is operable linked, too.

In another embodiment thereof the expression cassette does not comprise an additional transit peptide in case in Table I, application no. 17, in column 6 in the same line as SEQ ID NO. 35590 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 143742, or a homolog or fragment thereof, or a homolog thereof being depicted in Table I, application no. 17, column 8, in the same line as SEQ ID NO. 143742, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 143742 is depicted, plastidic is mentioned, or a mitochondrial transit peptide in case in Table I, application no. 17, in column 6 in the same line as SEQ ID NO. 143742 is depicted, mitochondric is mentioned; which is operable linked, too.

In another embodiment thereof the expression cassette does not comprise an additional transit peptide in case in Table I, application no. 17, in column 6 in the same line as SEQ ID NO. 143742 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 121111, or a homolog or fragment thereof, or a homolog thereof being depicted in Table I, application no. 17, column 8, in the same line as SEQ ID NO. 121111, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 121111 is depicted, plastidic is mentioned, or a mitochondrial transit peptide in case in Table I, application no. 17, in column 6 in the same line as SEQ ID NO. 121111 is depicted, mitochondric is mentioned; which is operable linked, too.

In another embodiment thereof the expression cassette does not comprise an additional transit peptide in case in Table I, application no. 17, in column 6 in the same line as SEQ ID NO. 121111 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 35733, or a homolog or fragment thereof, or a homolog thereof being depicted in Table I, application no. 17, column 8, in the same line as SEQ ID NO. 35733, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 35733 is depicted, plastidic is mentioned, or a mitochondrial transit peptide in case in Table I, application no. 17, in column 6 in the same line as SEQ ID NO. 35733 is depicted, mitochondric is mentioned; which is operable linked, too.

In another embodiment thereof the expression cassette does not comprise an additional transit peptide in case in Table I, application no. 17, in column 6 in the same line as SEQ ID NO. 35733 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 143761, or a homolog or fragment thereof, or a homolog thereof being depicted in Table I, application no. 17, column 8, in the same line as SEQ ID NO. 143761, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 143761 is depicted, plastidic is mentioned, or a mitochondrial transit peptide in case in Table I, application no. 17, in column 6 in the same line as SEQ ID NO. 143761 is depicted, mitochondric is mentioned; which is operable linked, too.

In another embodiment thereof the expression cassette does not comprise an additional transit peptide in case in Table I, application no. 17, in column 6 in the same line as SEQ ID NO. 143761 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 143771, or a homolog or fragment thereof, or a homolog thereof being depicted in Table I, application no. 17, column 8, in the same line as SEQ ID NO. 143771, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 143771 is depicted, plastidic is mentioned, or a mitochondrial transit peptide in case in Table I, application no. 17, in column 6 in the same line as SEQ ID NO. 143771 is depicted, mitochondric is mentioned; which is operable linked, too.

In another embodiment thereof the expression cassette does not comprise an additional transit peptide in case in Table I, application no. 17, in column 6 in the same line as SEQ ID NO. 143771 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7333, or a homolog or fragment thereof, or a homolog thereof being depicted in Table I, application no. 17, column 8, in the same line as SEQ ID NO. 7333, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 7333 is depicted, plastidic is mentioned, or a mitochondrial transit peptide in case in Table I, application no. 17, in column 6 in the same line as SEQ ID NO. 7333 is depicted, mitochondric is mentioned; which is operable linked, too.

In another embodiment thereof the expression cassette does not comprise an additional transit peptide in case in Table I, application no. 17, in column 6 in the same line as SEQ ID NO. 7333 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7686, or a homolog or fragment thereof, or a homolog thereof being depicted in Table I, application no. 17, column 8, in the same line as SEQ ID NO. 7686, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 7686 is depicted, plastidic is mentioned, or a mitochondrial transit peptide in case in Table I, application no. 17, in column 6 in the same line as SEQ ID NO. 7686 is depicted, mitochondric is mentioned; which is operable linked, too.

In another embodiment thereof the expression cassette does not comprise an additional transit peptide in case in Table I, application no. 17, in column 6 in the same line as SEQ ID NO. 7686 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 143817, or a homolog or fragment thereof, or a homolog thereof being depicted in Table I, application no. 17, column 8, in the same line as SEQ ID NO. 143817, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 143817 is depicted, plastidic is mentioned, or a mitochondrial transit peptide in case in Table I, application no. 17, in column 6 in the same line as SEQ ID NO. 143817 is depicted, mitochondric is mentioned; which is operable linked, too.

In another embodiment thereof the expression cassette does not comprise an additional transit peptide in case in Table I, application no. 17, in column 6 in the same line as SEQ ID NO. 143817 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 134402, or a homolog or fragment thereof, or a homolog thereof being depicted in Table I, application no. 17, column 8, in the same line as SEQ ID NO. 134402, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 134402 is depicted, plastidic is mentioned, or a mitochondrial transit peptide in case in Table I, application no. 17, in column 6 in the same line as SEQ ID NO. 134402 is depicted, mitochondric is mentioned; which is operable linked, too.

In another embodiment thereof the expression cassette does not comprise an additional transit peptide in case in Table I, application no. 17, in column 6 in the same line as SEQ ID NO. 134402 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 35967, or a homolog or fragment thereof, or a homolog thereof being depicted in Table I, application no. 17, column 8, in the same line as SEQ ID NO. 35967, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 35967 is depicted, plastidic is mentioned, or a mitochondrial transit peptide in case in Table I, application no. 17, in column 6 in the same line as SEQ ID NO. 35967 is depicted, mitochondric is mentioned; which is operable linked, too.

In another embodiment thereof the expression cassette does not comprise an additional transit peptide in case in Table I, application no. 17, in column 6 in the same line as SEQ ID NO. 35967 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 143901, or a homolog or fragment thereof, or a homolog thereof being depicted in Table I, application no. 17, column 8, in the same line as SEQ ID NO. 143901, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 143901 is depicted, plastidic is mentioned, or a mitochondrial transit peptide in case in Table I, application no. 17, in column 6 in the same line as SEQ ID NO. 143901 is depicted, mitochondric is mentioned; which is operable linked, too.

In another embodiment thereof the expression cassette does not comprise an additional transit peptide in case in Table I, application no. 17, in column 6 in the same line as SEQ ID NO. 143901 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 143954, or a homolog or fragment thereof, or a homolog thereof being depicted in Table I, application no. 17, column 8, in the same line as SEQ ID NO. 143954, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 143954 is depicted, plastidic is mentioned, or a mitochondrial transit peptide in case in Table I, application no. 17, in column 6 in the same line as SEQ ID NO. 143954 is depicted, mitochondric is mentioned; which is operable linked, too.

In another embodiment thereof the expression cassette does not comprise an additional transit peptide in case in Table I, application no. 17, in column 6 in the same line as SEQ ID NO. 143954 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 144207, or a homolog or fragment thereof, or a homolog thereof being depicted in Table I, application no. 17, column 8, in the same line as SEQ ID NO. 144207, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 144207 is depicted, plastidic is mentioned, or a mitochondrial transit peptide in case in Table I, application no. 17, in column 6 in the same line as SEQ ID NO. 144207 is depicted, mitochondric is mentioned; which is operable linked, too.

In another embodiment thereof the expression cassette does not comprise an additional transit peptide in case in Table I, application no. 17, in column 6 in the same line as SEQ ID NO. 144207 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 93678, or a homolog or fragment thereof, or a homolog thereof being depicted in Table I, application no. 17, column 8, in the same line as SEQ ID NO. 93678, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 93678 is depicted, plastidic is mentioned, or a mitochondrial transit peptide in case in Table I, application no. 17, in column 6 in the same line as SEQ ID NO. 93678 is depicted, mitochondric is mentioned; which is operable linked, too.

In another embodiment thereof the expression cassette does not comprise an additional transit peptide in case in Table I, application no. 17, in column 6 in the same line as SEQ ID NO. 93678 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 93793, or a homolog or fragment thereof, or a homolog thereof being depicted in Table I, application no. 17, column 8, in the same line as SEQ ID NO. 93793, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 93793 is depicted, plastidic is mentioned, or a mitochondrial transit peptide in case in Table I, application no. 17, in column 6 in the same line as SEQ ID NO. 93793 is depicted, mitochondric is mentioned; which is operable linked, too.

In another embodiment thereof the expression cassette does not comprise an additional transit peptide in case in Table I, application no. 17, in column 6 in the same line as SEQ ID NO. 93793 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 36114, or a homolog or fragment thereof, or a homolog thereof being depicted in Table I, application no. 17, column 8, in the same line as SEQ ID NO. 36114, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 36114 is depicted, plastidic is mentioned, or a mitochondrial transit peptide in case in Table I, application no. 17, in column 6 in the same line as SEQ ID NO. 36114 is depicted, mitochondric is mentioned; which is operable linked, too.

In another embodiment thereof the expression cassette does not comprise an additional transit peptide in case in Table I, application no. 17, in column 6 in the same line as SEQ ID NO. 36114 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 75807, or a homolog or fragment thereof, or a homolog thereof being depicted in Table I, application no. 17, column 8, in the same line as SEQ ID NO. 75807, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 75807 is depicted, plastidic is mentioned, or a mitochondrial transit peptide in case in Table I, application no. 17, in column 6 in the same line as SEQ ID NO. 75807 is depicted, mitochondric is mentioned; which is operable linked, too.

In another embodiment thereof the expression cassette does not comprise an additional transit peptide in case in Table I, application no. 17, in column 6 in the same line as SEQ ID NO. 75807 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 131359, or a homolog or fragment thereof, or a homolog thereof being depicted in Table I, application no. 17, column 8, in the same line as SEQ ID NO. 131359, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 131359 is depicted, plastidic is mentioned, or a mitochondrial transit peptide in case in Table I, application no. 17, in column 6 in the same line as SEQ ID NO. 131359 is depicted, mitochondric is mentioned; which is operable linked, too.

In another embodiment thereof the expression cassette does not comprise an additional transit peptide in case in Table I, application no. 17, in column 6 in the same line as SEQ ID NO. 131359 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 36489, or a homolog or fragment thereof, or a homolog thereof being depicted in Table I, application no. 17, column 8, in the same line as SEQ ID NO. 36489, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 36489 is depicted, plastidic is mentioned, or a mitochondrial transit peptide in case in Table I, application no. 17, in column 6 in the same line as SEQ ID NO. 36489 is depicted, mitochondric is mentioned; which is operable linked, too.

In another embodiment thereof the expression cassette does not comprise an additional transit peptide in case in Table I, application no. 17, in column 6 in the same line as SEQ ID NO. 36489 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 144479, or a homolog or fragment thereof, or a homolog thereof being depicted in Table I, application no. 17, column 8, in the same line as SEQ ID NO. 144479, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 144479 is depicted, plastidic is mentioned, or a mitochondrial transit peptide in case in Table I, application no. 17, in column 6 in the same line as SEQ ID NO. 144479 is depicted, mitochondric is mentioned; which is operable linked, too.

In another embodiment thereof the expression cassette does not comprise an additional transit peptide in case in Table I, application no. 17, in column 6 in the same line as SEQ ID NO. 144479 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 36809, or a homolog or fragment thereof, or a homolog thereof being depicted in Table I, application no. 17, column 8, in the same line as SEQ ID NO. 36809, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 36809 is depicted, plastidic is mentioned, or a mitochondrial transit peptide in case in Table I, application no. 17, in column 6 in the same line as SEQ ID NO. 36809 is depicted, mitochondric is mentioned; which is operable linked, too.

In another embodiment thereof the expression cassette does not comprise an additional transit peptide in case in Table I, application no. 17, in column 6 in the same line as SEQ ID NO. 36809 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7941, or a homolog or fragment thereof, or a homolog thereof being depicted in Table I, application no. 17, column 8, in the same line as SEQ ID NO. 7941, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 7941 is depicted, plastidic is mentioned, or a mitochondrial transit peptide in case in Table I, application no. 17, in column 6 in the same line as SEQ ID NO. 7941 is depicted, mitochondric is mentioned; which is operable linked, too.

In another embodiment thereof the expression cassette does not comprise an additional transit peptide in case in Table I, application no. 17, in column 6 in the same line as SEQ ID NO. 7941 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 124051, or a homolog or fragment thereof, or a homolog thereof being depicted in Table I, application no. 17, column 8, in the same line as SEQ ID NO. 124051, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 124051 is depicted, plastidic is mentioned, or a mitochondrial transit peptide in case in Table I, application no. 17, in column 6 in the same line as SEQ ID NO. 124051 is depicted, mitochondric is mentioned; which is operable linked, too.

In another embodiment thereof the expression cassette does not comprise an additional transit peptide in case in Table I, application no. 17, in column 6 in the same line as SEQ ID NO. 124051 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 76032, or a homolog or fragment thereof, or a homolog thereof being depicted in Table I, application no. 17, column 8, in the same line as SEQ ID NO. 76032, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 76032 is depicted, plastidic is mentioned, or a mitochondrial transit peptide in case in Table I, application no. 17, in column 6 in the same line as SEQ ID NO. 76032 is depicted, mitochondric is mentioned; which is operable linked, too.

In another embodiment thereof the expression cassette does not comprise an additional transit peptide in case in Table I, application no. 17, in column 6 in the same line as SEQ ID NO. 76032 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 114979, or a homolog or fragment thereof, or a homolog thereof being depicted in Table I, application no. 17, column 8, in the same line as SEQ ID NO. 114979, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 114979 is depicted, plastidic is mentioned, or a mitochondrial transit peptide in case in Table I, application no. 17, in column 6 in the same line as SEQ ID NO. 114979 is depicted, mitochondric is mentioned; which is operable linked, too.

In another embodiment thereof the expression cassette does not comprise an additional transit peptide in case in Table I, application no. 17, in column 6 in the same line as SEQ ID NO. 114979 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 144611, or a homolog or fragment thereof, or a homolog thereof being depicted in Table I, application no. 17, column 8, in the same line as SEQ ID NO. 144611, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 144611 is depicted, plastidic is mentioned, or a mitochondrial transit peptide in case in Table I, application no. 17, in column 6 in the same line as SEQ ID NO. 144611 is depicted, mitochondric is mentioned; which is operable linked, too.

In another embodiment thereof the expression cassette does not comprise an additional transit peptide in case in Table I, application no. 17, in column 6 in the same line as SEQ ID NO. 144611 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 36971, or a homolog or fragment thereof, or a homolog thereof being depicted in Table I, application no. 17, column 8, in the same line as SEQ ID NO. 36971, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 36971 is depicted, plastidic is mentioned, or a mitochondrial transit peptide in case in Table I, application no. 17, in column 6 in the same line as SEQ ID NO. 36971 is depicted, mitochondric is mentioned; which is operable linked, too.

In another embodiment thereof the expression cassette does not comprise an additional transit peptide in case in Table I, application no. 17, in column 6 in the same line as SEQ ID NO. 36971 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 37390, or a homolog or fragment thereof, or a homolog thereof being depicted in Table I, application no. 17, column 8, in the same line as SEQ ID NO. 37390, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 37390 is depicted, plastidic is mentioned, or a mitochondrial transit peptide in case in Table I, application no. 17, in column 6 in the same line as SEQ ID NO. 37390 is depicted, mitochondric is mentioned; which is operable linked, too.

In another embodiment thereof the expression cassette does not comprise an additional transit peptide in case in Table I, application no. 17, in column 6 in the same line as SEQ ID NO. 37390 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 37400, or a homolog or fragment thereof, or a homolog thereof being depicted in Table I, application no. 17, column 8, in the same line as SEQ ID NO. 37400, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 37400 is depicted, plastidic is mentioned, or a mitochondrial transit peptide in case in Table I, application no. 17, in column 6 in the same line as SEQ ID NO. 37400 is depicted, mitochondric is mentioned; which is operable linked, too.

In another embodiment thereof the expression cassette does not comprise an additional transit peptide in case in Table I, application no. 17, in column 6 in the same line as SEQ ID NO. 37400 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 144857, or a homolog or fragment thereof, or a homolog thereof being depicted in Table I, application no. 17, column 8, in the same line as SEQ ID NO. 144857, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 144857 is depicted, plastidic is mentioned, or a mitochondrial transit peptide in case in Table I, application no. 17, in column 6 in the same line as SEQ ID NO. 144857 is depicted, mitochondric is mentioned; which is operable linked, too.

In another embodiment thereof the expression cassette does not comprise an additional transit peptide in case in Table I, application no. 17, in column 6 in the same line as SEQ ID NO. 144857 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 112072, or a homolog or fragment thereof, or a homolog thereof being depicted in Table I, application no. 17, column 8, in the same line as SEQ ID NO. 112072, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 112072 is depicted, plastidic is mentioned, or a mitochondrial transit peptide in case in Table I, application no. 17, in column 6 in the same line as SEQ ID NO. 112072 is depicted, mitochondric is mentioned; which is operable linked, too.

In another embodiment thereof the expression cassette does not comprise an additional transit peptide in case in Table I, application no. 17, in column 6 in the same line as SEQ ID NO. 112072 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 112111, or a homolog or fragment thereof, or a homolog thereof being depicted in Table I, application no. 17, column 8, in the same line as SEQ ID NO. 112111, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 112111 is depicted, plastidic is mentioned, or a mitochondrial transit peptide in case in Table I, application no. 17, in column 6 in the same line as SEQ ID NO. 112111 is depicted, mitochondric is mentioned; which is operable linked, too.

In another embodiment thereof the expression cassette does not comprise an additional transit peptide in case in Table I, application no. 17, in column 6 in the same line as SEQ ID NO. 112111 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 98778, or a homolog or fragment thereof, or a homolog thereof being depicted in Table I, application no. 17, column 8, in the same line as SEQ ID NO. 98778, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 98778 is depicted, plastidic is mentioned, or a mitochondrial transit peptide in case in Table I, application no. 17, in column 6 in the same line as SEQ ID NO. 98778 is depicted, mitochondric is mentioned; which is operable linked, too.

In another embodiment thereof the expression cassette does not comprise an additional transit peptide in case in Table I, application no. 17, in column 6 in the same line as SEQ ID NO. 98778 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 144894, or a homolog or fragment thereof, or a homolog thereof being depicted in Table I, application no. 17, column 8, in the same line as SEQ ID NO. 144894, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 144894 is depicted, plastidic is mentioned, or a mitochondrial transit peptide in case in Table I, application no. 17, in column 6 in the same line as SEQ ID NO. 144894 is depicted, mitochondric is mentioned; which is operable linked, too.

In another embodiment thereof the expression cassette does not comprise an additional transit peptide in case in Table I, application no. 17, in column 6 in the same line as SEQ ID NO. 144894 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 37483, or a homolog or fragment thereof, or a homolog thereof being depicted in Table I, application no. 17, column 8, in the same line as SEQ ID NO. 37483, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 37483 is depicted, plastidic is mentioned, or a mitochondrial transit peptide in case in Table I, application no. 17, in column 6 in the same line as SEQ ID NO. 37483 is depicted, mitochondric is mentioned; which is operable linked, too.

In another embodiment thereof the expression cassette does not comprise an additional transit peptide in case in Table I, application no. 17, in column 6 in the same line as SEQ ID NO. 37483 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 76157, or a homolog or fragment thereof, or a homolog thereof being depicted in Table I, application no. 17, column 8, in the same line as SEQ ID NO. 76157, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 76157 is depicted, plastidic is mentioned, or a mitochondrial transit peptide in case in Table I, application no. 17, in column 6 in the same line as SEQ ID NO. 76157 is depicted, mitochondric is mentioned; which is operable linked, too.

In another embodiment thereof the expression cassette does not comprise an additional transit peptide in case in Table I, application no. 17, in column 6 in the same line as SEQ ID NO. 76157 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 134655, or a homolog or fragment thereof, or a homolog thereof being depicted in Table I, application no. 17, column 8, in the same line as SEQ ID NO. 134655, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 134655 is depicted, plastidic is mentioned, or a mitochondrial transit peptide in case in Table I, application no. 17, in column 6 in the same line as SEQ ID NO. 134655 is depicted, mitochondric is mentioned; which is operable linked, too.

In another embodiment thereof the expression cassette does not comprise an additional transit peptide in case in Table I, application no. 17, in column 6 in the same line as SEQ ID NO. 134655 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 124557, or a homolog or fragment thereof, or a homolog thereof being depicted in Table I, application no. 17, column 8, in the same line as SEQ ID NO. 124557, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 124557 is depicted, plastidic is mentioned, or a mitochondrial transit peptide in case in Table I, application no. 17, in column 6 in the same line as SEQ ID NO. 124557 is depicted, mitochondric is mentioned; which is operable linked, too.

In another embodiment thereof the expression cassette does not comprise an additional transit peptide in case in Table I, application no. 17, in column 6 in the same line as SEQ ID NO. 124557 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 145066, or a homolog or fragment thereof, or a homolog thereof being depicted in Table I, application no. 17, column 8, in the same line as SEQ ID NO. 145066, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 145066 is depicted, plastidic is mentioned, or a mitochondrial transit peptide in case in Table I, application no. 17, in column 6 in the same line as SEQ ID NO. 145066 is depicted, mitochondric is mentioned; which is operable linked, too.

In another embodiment thereof the expression cassette does not comprise an additional transit peptide in case in Table I, application no. 17, in column 6 in the same line as SEQ ID NO. 145066 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 134697, or a homolog or fragment thereof, or a homolog thereof being depicted in Table I, application no. 17, column 8, in the same line as SEQ ID NO. 134697, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 134697 is depicted, plastidic is mentioned, or a mitochondrial transit peptide in case in Table I, application no. 17, in column 6 in the same line as SEQ ID NO. 134697 is depicted, mitochondric is mentioned; which is operable linked, too.

In another embodiment thereof the expression cassette does not comprise an additional transit peptide in case in Table I, application no. 17, in column 6 in the same line as SEQ ID NO. 134697 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 131489, or a homolog or fragment thereof, or a homolog thereof being depicted in Table I, application no. 17, column 8, in the same line as SEQ ID NO. 131489, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 131489 is depicted, plastidic is mentioned, or a mitochondrial transit peptide in case in Table I, application no. 17, in column 6 in the same line as SEQ ID NO. 131489 is depicted, mitochondric is mentioned; which is operable linked, too.

In another embodiment thereof the expression cassette does not comprise an additional transit peptide in case in Table I, application no. 17, in column 6 in the same line as SEQ ID NO. 131489 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 145091, or a homolog or fragment thereof, or a homolog thereof being depicted in Table I, application no. 17, column 8, in the same line as SEQ ID NO. 145091, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 145091 is depicted, plastidic is mentioned, or a mitochondrial transit peptide in case in Table I, application no. 17, in column 6 in the same line as SEQ ID NO. 145091 is depicted, mitochondric is mentioned; which is operable linked, too.

In another embodiment thereof the expression cassette does not comprise an additional transit peptide in case in Table I, application no. 17, in column 6 in the same line as SEQ ID NO. 145091 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 134756, or a homolog or fragment thereof, or a homolog thereof being depicted in Table I, application no. 17, column 8, in the same line as SEQ ID NO. 134756, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 134756 is depicted, plastidic is mentioned, or a mitochondrial transit peptide in case in Table I, application no. 17, in column 6 in the same line as SEQ ID NO. 134756 is depicted, mitochondric is mentioned; which is operable linked, too.

In another embodiment thereof the expression cassette does not comprise an additional transit peptide in case in Table I, application no. 17, in column 6 in the same line as SEQ ID NO. 134756 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38266, or a homolog or fragment thereof, or a homolog thereof being depicted in Table I, application no. 17, column 8, in the same line as SEQ ID NO. 38266, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 38266 is depicted, plastidic is mentioned, or a mitochondrial transit peptide in case in Table I, application no. 17, in column 6 in the same line as SEQ ID NO. 38266 is depicted, mitochondric is mentioned; which is operable linked, too.

In another embodiment thereof the expression cassette does not comprise an additional transit peptide in case in Table I, application no. 17, in column 6 in the same line as SEQ ID NO. 38266 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38289, or a homolog or fragment thereof, or a homolog thereof being depicted in Table I, application no. 17, column 8, in the same line as SEQ ID NO. 38289, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 38289 is depicted, plastidic is mentioned, or a mitochondrial transit peptide in case in Table I, application no. 17, in column 6 in the same line as SEQ ID NO. 38289 is depicted, mitochondric is mentioned; which is operable linked, too.

In another embodiment thereof the expression cassette does not comprise an additional transit peptide in case in Table I, application no. 17, in column 6 in the same line as SEQ ID NO. 38289 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115056, or a homolog or fragment thereof, or a homolog thereof being depicted in Table I, application no. 17, column 8, in the same line as SEQ ID NO. 115056, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 115056 is depicted, plastidic is mentioned, or a mitochondrial transit peptide in case in Table I, application no. 17, in column 6 in the same line as SEQ ID NO. 115056 is depicted, mitochondric is mentioned; which is operable linked, too.

In another embodiment thereof the expression cassette does not comprise an additional transit peptide in case in Table I, application no. 17, in column 6 in the same line as SEQ ID NO. 115056 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7947, or a homolog or fragment thereof, or a homolog thereof being depicted in Table I, application no. 17, column 8, in the same line as SEQ ID NO. 7947, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 7947 is depicted, plastidic is mentioned, or a mitochondrial transit peptide in case in Table I, application no. 17, in column 6 in the same line as SEQ ID NO. 7947 is depicted, mitochondric is mentioned; which is operable linked, too.

In another embodiment thereof the expression cassette does not comprise an additional transit peptide in case in Table I, application no. 17, in column 6 in the same line as SEQ ID NO. 7947 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 145142, or a homolog or fragment thereof, or a homolog thereof being depicted in Table I, application no. 17, column 8, in the same line as SEQ ID NO. 145142, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 145142 is depicted, plastidic is mentioned, or a mitochondrial transit peptide in case in Table I, application no. 17, in column 6 in the same line as SEQ ID NO. 145142 is depicted, mitochondric is mentioned; which is operable linked, too.

In another embodiment thereof the expression cassette does not comprise an additional transit peptide in case in Table I, application no. 17, in column 6 in the same line as SEQ ID NO. 145142 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78138, or a homolog or fragment thereof, or a homolog thereof being depicted in Table I, application no. 17, column 8, in the same line as SEQ ID NO. 78138, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 78138 is depicted, plastidic is mentioned, or a mitochondrial transit peptide in case in Table I, application no. 17, in column 6 in the same line as SEQ ID NO. 78138 is depicted, mitochondric is mentioned; which is operable linked, too.

In another embodiment thereof the expression cassette does not comprise an additional transit peptide in case in Table I, application no. 17, in column 6 in the same line as SEQ ID NO. 78138 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78154, or a homolog or fragment thereof, or a homolog thereof being depicted in Table I, application no. 17, column 8, in the same line as SEQ ID NO. 78154, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 78154 is depicted, plastidic is mentioned, or a mitochondrial transit peptide in case in Table I, application no. 17, in column 6 in the same line as SEQ ID NO. 78154 is depicted, mitochondric is mentioned; which is operable linked, too.

In another embodiment thereof the expression cassette does not comprise an additional transit peptide in case in Table I, application no. 17, in column 6 in the same line as SEQ ID NO. 78154 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38300, or a homolog or fragment thereof, or a homolog thereof being depicted in Table I, application no. 17, column 8, in the same line as SEQ ID NO. 38300, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 38300 is depicted, plastidic is mentioned, or a mitochondrial transit peptide in case in Table I, application no. 17, in column 6 in the same line as SEQ ID NO. 38300 is depicted, mitochondric is mentioned; which is operable linked, too.

In another embodiment thereof the expression cassette does not comprise an additional transit peptide in case in Table I, application no. 17, in column 6 in the same line as SEQ ID NO. 38300 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 124571, or a homolog or fragment thereof, or a homolog thereof being depicted in Table I, application no. 17, column 8, in the same line as SEQ ID NO. 124571, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 124571 is depicted, plastidic is mentioned, or a mitochondrial transit peptide in case in Table I, application no. 17, in column 6 in the same line as SEQ ID NO. 124571 is depicted, mitochondric is mentioned; which is operable linked, too.

In another embodiment thereof the expression cassette does not comprise an additional transit peptide in case in Table I, application no. 17, in column 6 in the same line as SEQ ID NO. 124571 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38345, or a homolog or fragment thereof, or a homolog thereof being depicted in Table I, application no. 17, column 8, in the same line as SEQ ID NO. 38345, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 38345 is depicted, plastidic is mentioned, or a mitochondrial transit peptide in case in Table I, application no. 17, in column 6 in the same line as SEQ ID NO. 38345 is depicted, mitochondric is mentioned; which is operable linked, too.

In another embodiment thereof the expression cassette does not comprise an additional transit peptide in case in Table I, application no. 17, in column 6 in the same line as SEQ ID NO. 38345 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78265, or a homolog or fragment thereof, or a homolog thereof being depicted in Table I, application no. 17, column 8, in the same line as SEQ ID NO. 78265, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 78265 is depicted, plastidic is mentioned, or a mitochondrial transit peptide in case in Table I, application no. 17, in column 6 in the same line as SEQ ID NO. 78265 is depicted, mitochondric is mentioned; which is operable linked, too.

In another embodiment thereof the expression cassette does not comprise an additional transit peptide in case in Table I, application no. 17, in column 6 in the same line as SEQ ID NO. 78265 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78753, or a homolog or fragment thereof, or a homolog thereof being depicted in Table I, application no. 17, column 8, in the same line as SEQ ID NO. 78753, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 78753 is depicted, plastidic is mentioned, or a mitochondrial transit peptide in case in Table I, application no. 17, in column 6 in the same line as SEQ ID NO. 78753 is depicted, mitochondric is mentioned; which is operable linked, too.

In another embodiment thereof the expression cassette does not comprise an additional transit peptide in case in Table I, application no. 17, in column 6 in the same line as SEQ ID NO. 78753 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78852, or a homolog or fragment thereof, or a homolog thereof being depicted in Table I, application no. 17, column 8, in the same line as SEQ ID NO. 78852, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 78852 is depicted, plastidic is mentioned, or a mitochondrial transit peptide in case in Table I, application no. 17, in column 6 in the same line as SEQ ID NO. 78852 is depicted, mitochondric is mentioned; which is operable linked, too.

In another embodiment thereof the expression cassette does not comprise an additional transit peptide in case in Table I, application no. 17, in column 6 in the same line as SEQ ID NO. 78852 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78883, or a homolog or fragment thereof, or a homolog thereof being depicted in Table I, application no. 17, column 8, in the same line as SEQ ID NO. 78883, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 78883 is depicted, plastidic is mentioned, or a mitochondrial transit peptide in case in Table I, application no. 17, in column 6 in the same line as SEQ ID NO. 78883 is depicted, mitochondric is mentioned; which is operable linked, too.

In another embodiment thereof the expression cassette does not comprise an additional transit peptide in case in Table I, application no. 17, in column 6 in the same line as SEQ ID NO. 78883 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 99196, or a homolog or fragment thereof, or a homolog thereof being depicted in Table I, application no. 17, column 8, in the same line as SEQ ID NO. 99196, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 99196 is depicted, plastidic is mentioned, or a mitochondrial transit peptide in case in Table I, application no. 17, in column 6 in the same line as SEQ ID NO. 99196 is depicted, mitochondric is mentioned; which is operable linked, too.

In another embodiment thereof the expression cassette does not comprise an additional transit peptide in case in Table I, application no. 17, in column 6 in the same line as SEQ ID NO. 99196 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 145180, or a homolog or fragment thereof, or a homolog thereof being depicted in Table I, application no. 17, column 8, in the same line as SEQ ID NO. 145180, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 145180 is depicted, plastidic is mentioned, or a mitochondrial transit peptide in case in Table I, application no. 17, in column 6 in the same line as SEQ ID NO. 145180 is depicted, mitochondric is mentioned; which is operable linked, too.

In another embodiment thereof the expression cassette does not comprise an additional transit peptide in case in Table I, application no. 17, in column 6 in the same line as SEQ ID NO. 145180 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 8033, or a homolog or fragment thereof, or a homolog thereof being depicted in Table I, application no. 17, column 8, in the same line as SEQ ID NO. 8033, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 8033 is depicted, plastidic is mentioned, or a mitochondrial transit peptide in case in Table I, application no. 17, in column 6 in the same line as SEQ ID NO. 8033 is depicted, mitochondric is mentioned; which is operable linked, too.

In another embodiment thereof the expression cassette does not comprise an additional transit peptide in case in Table I, application no. 17, in column 6 in the same line as SEQ ID NO. 8033 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38573, or a homolog or fragment thereof, or a homolog thereof being depicted in Table I, application no. 17, column 8, in the same line as SEQ ID NO. 38573, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 38573 is depicted, plastidic is mentioned, or a mitochondrial transit peptide in case in Table I, application no. 17, in column 6 in the same line as SEQ ID NO. 38573 is depicted, mitochondric is mentioned; which is operable linked, too.

In another embodiment thereof the expression cassette does not comprise an additional transit peptide in case in Table I, application no. 17, in column 6 in the same line as SEQ ID NO. 38573 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 8363, or a homolog or fragment thereof, or a homolog thereof being depicted in Table I, application no. 17, column 8, in the same line as SEQ ID NO. 8363, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 8363 is depicted, plastidic is mentioned, or a mitochondrial transit peptide in case in Table I, application no. 17, in column 6 in the same line as SEQ ID NO. 8363 is depicted, mitochondric is mentioned; which is operable linked, too.

In another embodiment thereof the expression cassette does not comprise an additional transit peptide in case in Table I, application no. 17, in column 6 in the same line as SEQ ID NO. 8363 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 134783, or a homolog or fragment thereof, or a homolog thereof being depicted in Table I, application no. 17, column 8, in the same line as SEQ ID NO. 134783, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 134783 is depicted, plastidic is mentioned, or a mitochondrial transit peptide in case in Table I, application no. 17, in column 6 in the same line as SEQ ID NO. 134783 is depicted, mitochondric is mentioned; which is operable linked, too.

In another embodiment thereof the expression cassette does not comprise an additional transit peptide in case in Table I, application no. 17, in column 6 in the same line as SEQ ID NO. 134783 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 145411, or a homolog or fragment thereof, or a homolog thereof being depicted in Table I, application no. 17, column 8, in the same line as SEQ ID NO. 145411, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 145411 is depicted, plastidic is mentioned, or a mitochondrial transit peptide in case in Table I, application no. 17, in column 6 in the same line as SEQ ID NO. 145411 is depicted, mitochondric is mentioned; which is operable linked, too.

In another embodiment thereof the expression cassette does not comprise an additional transit peptide in case in Table I, application no. 17, in column 6 in the same line as SEQ ID NO. 145411 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 79056, or a homolog or fragment thereof, or a homolog thereof being depicted in Table I, application no. 17, column 8, in the same line as SEQ ID NO. 79056, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 79056 is depicted, plastidic is mentioned, or a mitochondrial transit peptide in case in Table I, application no. 17, in column 6 in the same line as SEQ ID NO. 79056 is depicted, mitochondric is mentioned; which is operable linked, too.

In another embodiment thereof the expression cassette does not comprise an additional transit peptide in case in Table I, application no. 17, in column 6 in the same line as SEQ ID NO. 79056 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 79056, or a homolog or fragment thereof, or a homolog thereof being depicted in Table I, application no. 17, column 8, in the same line as SEQ ID NO. 79056, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 79056 is depicted, plastidic is mentioned, or a mitochondrial transit peptide in case in Table I, application no. 17, in column 6 in the same line as SEQ ID NO. 79056 is depicted, mitochondric is mentioned; which is operable linked, too.

In another embodiment thereof the expression cassette does not comprise an additional transit peptide in case in Table I, application no. 17, in column 6 in the same line as SEQ ID NO. 79056 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38767, or a homolog or fragment thereof, or a homolog thereof being depicted in Table I, application no. 17, column 8, in the same line as SEQ ID NO. 38767, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 38767 is depicted, plastidic is mentioned, or a mitochondrial transit peptide in case in Table I, application no. 17, in column 6 in the same line as SEQ ID NO. 38767 is depicted, mitochondric is mentioned; which is operable linked, too.

In another embodiment thereof the expression cassette does not comprise an additional transit peptide in case in Table I, application no. 17, in column 6 in the same line as SEQ ID NO. 38767 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 131799, or a homolog or fragment thereof, or a homolog thereof being depicted in Table I, application no. 17, column 8, in the same line as SEQ ID NO. 131799, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 131799 is depicted, plastidic is mentioned, or a mitochondrial transit peptide in case in Table I, application no. 17, in column 6 in the same line as SEQ ID NO. 131799 is depicted, mitochondric is mentioned; which is operable linked, too.

In another embodiment thereof the expression cassette does not comprise an additional transit peptide in case in Table I, application no. 17, in column 6 in the same line as SEQ ID NO. 131799 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 134864, or a homolog or fragment thereof, or a homolog thereof being depicted in Table I, application no. 17, column 8, in the same line as SEQ ID NO. 134864, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 134864 is depicted, plastidic is mentioned, or a mitochondrial transit peptide in case in Table I, application no. 17, in column 6 in the same line as SEQ ID NO. 134864 is depicted, mitochondric is mentioned; which is operable linked, too.

In another embodiment thereof the expression cassette does not comprise an additional transit peptide in case in Table I, application no. 17, in column 6 in the same line as SEQ ID NO. 134864 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 79217, or a homolog or fragment thereof, or a homolog thereof being depicted in Table I, application no. 17, column 8, in the same line as SEQ ID NO. 79217, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 79217 is depicted, plastidic is mentioned, or a mitochondrial transit peptide in case in Table I, application no. 17, in column 6 in the same line as SEQ ID NO. 79217 is depicted, mitochondric is mentioned; which is operable linked, too.

In another embodiment thereof the expression cassette does not comprise an additional transit peptide in case in Table I, application no. 17, in column 6 in the same line as SEQ ID NO. 79217 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 8920, or a homolog or fragment thereof, or a homolog thereof being depicted in Table I, application no. 17, column 8, in the same line as SEQ ID NO. 8920, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 8920 is depicted, plastidic is mentioned, or a mitochondrial transit peptide in case in Table I, application no. 17, in column 6 in the same line as SEQ ID NO. 8920 is depicted, mitochondric is mentioned; which is operable linked, too.

In another embodiment thereof the expression cassette does not comprise an additional transit peptide in case in Table I, application no. 17, in column 6 in the same line as SEQ ID NO. 8920 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 112193, or a homolog or fragment thereof, or a homolog thereof being depicted in Table I, application no. 17, column 8, in the same line as SEQ ID NO. 112193, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 112193 is depicted, plastidic is mentioned, or a mitochondrial transit peptide in case in Table I, application no. 17, in column 6 in the same line as SEQ ID NO. 112193 is depicted, mitochondric is mentioned; which is operable linked, too.

In another embodiment thereof the expression cassette does not comprise an additional transit peptide in case in Table I, application no. 17, in column 6 in the same line as SEQ ID NO. 112193 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 104277, or a homolog or fragment thereof, or a homolog thereof being depicted in Table I, application no. 17, column 8, in the same line as SEQ ID NO. 104277, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 104277 is depicted, plastidic is mentioned, or a mitochondrial transit peptide in case in Table I, application no. 17, in column 6 in the same line as SEQ ID NO. 104277 is depicted, mitochondric is mentioned; which is operable linked, too.

In another embodiment thereof the expression cassette does not comprise an additional transit peptide in case in Table I, application no. 17, in column 6 in the same line as SEQ ID NO. 104277 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38947, or a homolog or fragment thereof, or a homolog thereof being depicted in Table I, application no. 17, column 8, in the same line as SEQ ID NO. 38947, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 38947 is depicted, plastidic is mentioned, or a mitochondrial transit peptide in case in Table I, application no. 17, in column 6 in the same line as SEQ ID NO. 38947 is depicted, mitochondric is mentioned; which is operable linked, too.

In another embodiment thereof the expression cassette does not comprise an additional transit peptide in case in Table I, application no. 17, in column 6 in the same line as SEQ ID NO. 38947 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39002, or a homolog or fragment thereof, or a homolog thereof being depicted in Table I, application no. 17, column 8, in the same line as SEQ ID NO. 39002, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 39002 is depicted, plastidic is mentioned, or a mitochondrial transit peptide in case in Table I, application no. 17, in column 6 in the same line as SEQ ID NO. 39002 is depicted, mitochondric is mentioned; which is operable linked, too.

In another embodiment thereof the expression cassette does not comprise an additional transit peptide in case in Table I, application no. 17, in column 6 in the same line as SEQ ID NO. 39002 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 125085, or a homolog or fragment thereof, or a homolog thereof being depicted in Table I, application no. 17, column 8, in the same line as SEQ ID NO. 125085, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 125085 is depicted, plastidic is mentioned, or a mitochondrial transit peptide in case in Table I, application no. 17, in column 6 in the same line as SEQ ID NO. 125085 is depicted, mitochondric is mentioned; which is operable linked, too.

In another embodiment thereof the expression cassette does not comprise an additional transit peptide in case in Table I, application no. 17, in column 6 in the same line as SEQ ID NO. 125085 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 79279, or a homolog or fragment thereof, or a homolog thereof being depicted in Table I, application no. 17, column 8, in the same line as SEQ ID NO. 79279, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 79279 is depicted, plastidic is mentioned, or a mitochondrial transit peptide in case in Table I, application no. 17, in column 6 in the same line as SEQ ID NO. 79279 is depicted, mitochondric is mentioned; which is operable linked, too.

In another embodiment thereof the expression cassette does not comprise an additional transit peptide in case in Table I, application no. 17, in column 6 in the same line as SEQ ID NO. 79279 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 99620, or a homolog or fragment thereof, or a homolog thereof being depicted in Table I, application no. 17, column 8, in the same line as SEQ ID NO. 99620, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 99620 is depicted, plastidic is mentioned, or a mitochondrial transit peptide in case in Table I, application no. 17, in column 6 in the same line as SEQ ID NO. 99620 is depicted, mitochondric is mentioned; which is operable linked, too.

In another embodiment thereof the expression cassette does not comprise an additional transit peptide in case in Table I, application no. 17, in column 6 in the same line as SEQ ID NO. 99620 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 145692, or a homolog or fragment thereof, or a homolog thereof being depicted in Table I, application no. 17, column 8, in the same line as SEQ ID NO. 145692, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 145692 is depicted, plastidic is mentioned, or a mitochondrial transit peptide in case in Table I, application no. 17, in column 6 in the same line as SEQ ID NO. 145692 is depicted, mitochondric is mentioned; which is operable linked, too.

In another embodiment thereof the expression cassette does not comprise an additional transit peptide in case in Table I, application no. 17, in column 6 in the same line as SEQ ID NO. 145692 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 9156, or a homolog or fragment thereof, or a homolog thereof being depicted in Table I, application no. 17, column 8, in the same line as SEQ ID NO. 9156, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 9156 is depicted, plastidic is mentioned, or a mitochondrial transit peptide in case in Table I, application no. 17, in column 6 in the same line as SEQ ID NO. 9156 is depicted, mitochondric is mentioned; which is operable linked, too.

In another embodiment thereof the expression cassette does not comprise an additional transit peptide in case in Table I, application no. 17, in column 6 in the same line as SEQ ID NO. 9156 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39219, or a homolog or fragment thereof, or a homolog thereof being depicted in Table I, application no. 17, column 8, in the same line as SEQ ID NO. 39219, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 39219 is depicted, plastidic is mentioned, or a mitochondrial transit peptide in case in Table I, application no. 17, in column 6 in the same line as SEQ ID NO. 39219 is depicted, mitochondric is mentioned; which is operable linked, too.

In another embodiment thereof the expression cassette does not comprise an additional transit peptide in case in Table I, application no. 17, in column 6 in the same line as SEQ ID NO. 39219 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 146204, or a homolog or fragment thereof, or a homolog thereof being depicted in Table I, application no. 17, column 8, in the same line as SEQ ID NO. 146204, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 146204 is depicted, plastidic is mentioned, or a mitochondrial transit peptide in case in Table I, application no. 17, in column 6 in the same line as SEQ ID NO. 146204 is depicted, mitochondric is mentioned; which is operable linked, too.

In another embodiment thereof the expression cassette does not comprise an additional transit peptide in case in Table I, application no. 17, in column 6 in the same line as SEQ ID NO. 146204 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 146238, or a homolog or fragment thereof, or a homolog thereof being depicted in Table I, application no. 17, column 8, in the same line as SEQ ID NO. 146238, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 146238 is depicted, plastidic is mentioned, or a mitochondrial transit peptide in case in Table I, application no. 17, in column 6 in the same line as SEQ ID NO. 146238 is depicted, mitochondric is mentioned; which is operable linked, too.

In another embodiment thereof the expression cassette does not comprise an additional transit peptide in case in Table I, application no. 17, in column 6 in the same line as SEQ ID NO. 146238 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39237, or a homolog or fragment thereof, or a homolog thereof being depicted in Table I, application no. 17, column 8, in the same line as SEQ ID NO. 39237, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 39237 is depicted, plastidic is mentioned, or a mitochondrial transit peptide in case in Table I, application no. 17, in column 6 in the same line as SEQ ID NO. 39237 is depicted, mitochondric is mentioned; which is operable linked, too.

In another embodiment thereof the expression cassette does not comprise an additional transit peptide in case in Table I, application no. 17, in column 6 in the same line as SEQ ID NO. 39237 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39255, or a homolog or fragment thereof, or a homolog thereof being depicted in Table I, application no. 17, column 8, in the same line as SEQ ID NO. 39255, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 39255 is depicted, plastidic is mentioned, or a mitochondrial transit peptide in case in Table I, application no. 17, in column 6 in the same line as SEQ ID NO. 39255 is depicted, mitochondric is mentioned; which is operable linked, too.

In another embodiment thereof the expression cassette does not comprise an additional transit peptide in case in Table I, application no. 17, in column 6 in the same line as SEQ ID NO. 39255 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39300, or a homolog or fragment thereof, or a homolog thereof being depicted in Table I, application no. 17, column 8, in the same line as SEQ ID NO. 39300, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 39300 is depicted, plastidic is mentioned, or a mitochondrial transit peptide in case in Table I, application no. 17, in column 6 in the same line as SEQ ID NO. 39300 is depicted, mitochondric is mentioned; which is operable linked, too.

In another embodiment thereof the expression cassette does not comprise an additional transit peptide in case in Table I, application no. 17, in column 6 in the same line as SEQ ID NO. 39300 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 146261, or a homolog or fragment thereof, or a homolog thereof being depicted in Table I, application no. 17, column 8, in the same line as SEQ ID NO. 146261, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 146261 is depicted, plastidic is mentioned, or a mitochondrial transit peptide in case in Table I, application no. 17, in column 6 in the same line as SEQ ID NO. 146261 is depicted, mitochondric is mentioned; which is operable linked, too.

In another embodiment thereof the expression cassette does not comprise an additional transit peptide in case in Table I, application no. 17, in column 6 in the same line as SEQ ID NO. 146261 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40329, or a homolog or fragment thereof, or a homolog thereof being depicted in Table I, application no. 17, column 8, in the same line as SEQ ID NO. 40329, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 40329 is depicted, plastidic is mentioned, or a mitochondrial transit peptide in case in Table I, application no. 17, in column 6 in the same line as SEQ ID NO. 40329 is depicted, mitochondric is mentioned; which is operable linked, too.

In another embodiment thereof the expression cassette does not comprise an additional transit peptide in case in Table I, application no. 17, in column 6 in the same line as SEQ ID NO. 40329 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40383, or a homolog or fragment thereof, or a homolog thereof being depicted in Table I, application no. 17, column 8, in the same line as SEQ ID NO. 40383, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 40383 is depicted, plastidic is mentioned, or a mitochondrial transit peptide in case in Table I, application no. 17, in column 6 in the same line as SEQ ID NO. 40383 is depicted, mitochondric is mentioned; which is operable linked, too.

In another embodiment thereof the expression cassette does not comprise an additional transit peptide in case in Table I, application no. 17, in column 6 in the same line as SEQ ID NO. 40383 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 99898, or a homolog or fragment thereof, or a homolog thereof being depicted in Table I, application no. 17, column 8, in the same line as SEQ ID NO. 99898, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 99898 is depicted, plastidic is mentioned, or a mitochondrial transit peptide in case in Table I, application no. 17, in column 6 in the same line as SEQ ID NO. 99898 is depicted, mitochondric is mentioned; which is operable linked, too.

In another embodiment thereof the expression cassette does not comprise an additional transit peptide in case in Table I, application no. 17, in column 6 in the same line as SEQ ID NO. 99898 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40665, or a homolog or fragment thereof, or a homolog thereof being depicted in Table I, application no. 17, column 8, in the same line as SEQ ID NO. 40665, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 40665 is depicted, plastidic is mentioned, or a mitochondrial transit peptide in case in Table I, application no. 17, in column 6 in the same line as SEQ ID NO. 40665 is depicted, mitochondric is mentioned; which is operable linked, too.

In another embodiment thereof the expression cassette does not comprise an additional transit peptide in case in Table I, application no. 17, in column 6 in the same line as SEQ ID NO. 40665 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40726, or a homolog or fragment thereof, or a homolog thereof being depicted in Table I, application no. 17, column 8, in the same line as SEQ ID NO. 40726, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 40726 is depicted, plastidic is mentioned, or a mitochondrial transit peptide in case in Table I, application no. 17, in column 6 in the same line as SEQ ID NO. 40726 is depicted, mitochondric is mentioned; which is operable linked, too.

In another embodiment thereof the expression cassette does not comprise an additional transit peptide in case in Table I, application no. 17, in column 6 in the same line as SEQ ID NO. 40726 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 146288, or a homolog or fragment thereof, or a homolog thereof being depicted in Table I, application no. 17, column 8, in the same line as SEQ ID NO. 146288, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 146288 is depicted, plastidic is mentioned, or a mitochondrial transit peptide in case in Table I, application no. 17, in column 6 in the same line as SEQ ID NO. 146288 is depicted, mitochondric is mentioned; which is operable linked, too.

In another embodiment thereof the expression cassette does not comprise an additional transit peptide in case in Table I, application no. 17, in column 6 in the same line as SEQ ID NO. 146288 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 125226, or a homolog or fragment thereof, or a homolog thereof being depicted in Table I, application no. 17, column 8, in the same line as SEQ ID NO. 125226, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 125226 is depicted, plastidic is mentioned, or a mitochondrial transit peptide in case in Table I, application no. 17, in column 6 in the same line as SEQ ID NO. 125226 is depicted, mitochondric is mentioned; which is operable linked, too.

In another embodiment thereof the expression cassette does not comprise an additional transit peptide in case in Table I, application no. 17, in column 6 in the same line as SEQ ID NO. 125226 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 146294, or a homolog or fragment thereof, or a homolog thereof being depicted in Table I, application no. 17, column 8, in the same line as SEQ ID NO. 146294, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 146294 is depicted, plastidic is mentioned, or a mitochondrial transit peptide in case in Table I, application no. 17, in column 6 in the same line as SEQ ID NO. 146294 is depicted, mitochondric is mentioned; which is operable linked, too.

In another embodiment thereof the expression cassette does not comprise an additional transit peptide in case in Table I, application no. 17, in column 6 in the same line as SEQ ID NO. 146294 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 9244, or a homolog or fragment thereof, or a homolog thereof being depicted in Table I, application no. 17, column 8, in the same line as SEQ ID NO. 9244, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 9244 is depicted, plastidic is mentioned, or a mitochondrial transit peptide in case in Table I, application no. 17, in column 6 in the same line as SEQ ID NO. 9244 is depicted, mitochondric is mentioned; which is operable linked, too.

In another embodiment thereof the expression cassette does not comprise an additional transit peptide in case in Table I, application no. 17, in column 6 in the same line as SEQ ID NO. 9244 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115655, or a homolog or fragment thereof, or a homolog thereof being depicted in Table I, application no. 17, column 8, in the same line as SEQ ID NO. 115655, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 115655 is depicted, plastidic is mentioned, or a mitochondrial transit peptide in case in Table I, application no. 17, in column 6 in the same line as SEQ ID NO. 115655 is depicted, mitochondric is mentioned; which is operable linked, too.

In another embodiment thereof the expression cassette does not comprise an additional transit peptide in case in Table I, application no. 17, in column 6 in the same line as SEQ ID NO. 115655 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 100105, or a homolog or fragment thereof, or a homolog thereof being depicted in Table I, application no. 17, column 8, in the same line as SEQ ID NO. 100105, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 100105 is depicted, plastidic is mentioned, or a mitochondrial transit peptide in case in Table I, application no. 17, in column 6 in the same line as SEQ ID NO. 100105 is depicted, mitochondric is mentioned; which is operable linked, too.

In another embodiment thereof the expression cassette does not comprise an additional transit peptide in case in Table I, application no. 17, in column 6 in the same line as SEQ ID NO. 100105 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 80756, or a homolog or fragment thereof, or a homolog thereof being depicted in Table I, application no. 17, column 8, in the same line as SEQ ID NO. 80756, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 80756 is depicted, plastidic is mentioned, or a mitochondrial transit peptide in case in Table I, application no. 17, in column 6 in the same line as SEQ ID NO. 80756 is depicted, mitochondric is mentioned; which is operable linked, too.

In another embodiment thereof the expression cassette does not comprise an additional transit peptide in case in Table I, application no. 17, in column 6 in the same line as SEQ ID NO. 80756 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40741, or a homolog or fragment thereof, or a homolog thereof being depicted in Table I, application no. 17, column 8, in the same line as SEQ ID NO. 40741, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 40741 is depicted, plastidic is mentioned, or a mitochondrial transit peptide in case in Table I, application no. 17, in column 6 in the same line as SEQ ID NO. 40741 is depicted, mitochondric is mentioned; which is operable linked, too.

In another embodiment thereof the expression cassette does not comprise an additional transit peptide in case in Table I, application no. 17, in column 6 in the same line as SEQ ID NO. 40741 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 80906, or a homolog or fragment thereof, or a homolog thereof being depicted in Table I, application no. 17, column 8, in the same line as SEQ ID NO. 80906, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 80906 is depicted, plastidic is mentioned, or a mitochondrial transit peptide in case in Table I, application no. 17, in column 6 in the same line as SEQ ID NO. 80906 is depicted, mitochondric is mentioned; which is operable linked, too.

In another embodiment thereof the expression cassette does not comprise an additional transit peptide in case in Table I, application no. 17, in column 6 in the same line as SEQ ID NO. 80906 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 81075, or a homolog or fragment thereof, or a homolog thereof being depicted in Table I, application no. 17, column 8, in the same line as SEQ ID NO. 81075, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 81075 is depicted, plastidic is mentioned, or a mitochondrial transit peptide in case in Table I, application no. 17, in column 6 in the same line as SEQ ID NO. 81075 is depicted, mitochondric is mentioned; which is operable linked, too.

In another embodiment thereof the expression cassette does not comprise an additional transit peptide in case in Table I, application no. 17, in column 6 in the same line as SEQ ID NO. 81075 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 81451, or a homolog or fragment thereof, or a homolog thereof being depicted in Table I, application no. 17, column 8, in the same line as SEQ ID NO. 81451, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 81451 is depicted, plastidic is mentioned, or a mitochondrial transit peptide in case in Table I, application no. 17, in column 6 in the same line as SEQ ID NO. 81451 is depicted, mitochondric is mentioned; which is operable linked, too.

In another embodiment thereof the expression cassette does not comprise an additional transit peptide in case in Table I, application no. 17, in column 6 in the same line as SEQ ID NO. 81451 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115719, or a homolog or fragment thereof, or a homolog thereof being depicted in Table I, application no. 17, column 8, in the same line as SEQ ID NO. 115719, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 115719 is depicted, plastidic is mentioned, or a mitochondrial transit peptide in case in Table I, application no. 17, in column 6 in the same line as SEQ ID NO. 115719 is depicted, mitochondric is mentioned; which is operable linked, too.

In another embodiment thereof the expression cassette does not comprise an additional transit peptide in case in Table I, application no. 17, in column 6 in the same line as SEQ ID NO. 115719 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 81576, or a homolog or fragment thereof, or a homolog thereof being depicted in Table I, application no. 17, column 8, in the same line as SEQ ID NO. 81576, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 81576 is depicted, plastidic is mentioned, or a mitochondrial transit peptide in case in Table I, application no. 17, in column 6 in the same line as SEQ ID NO. 81576 is depicted, mitochondric is mentioned; which is operable linked, too.

In another embodiment thereof the expression cassette does not comprise an additional transit peptide in case in Table I, application no. 17, in column 6 in the same line as SEQ ID NO. 81576 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 81935, or a homolog or fragment thereof, or a homolog thereof being depicted in Table I, application no. 17, column 8, in the same line as SEQ ID NO. 81935, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 81935 is depicted, plastidic is mentioned, or a mitochondrial transit peptide in case in Table I, application no. 17, in column 6 in the same line as SEQ ID NO. 81935 is depicted, mitochondric is mentioned; which is operable linked, too.

In another embodiment thereof the expression cassette does not comprise an additional transit peptide in case in Table I, application no. 17, in column 6 in the same line as SEQ ID NO. 81935 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 9333, or a homolog or fragment thereof, or a homolog thereof being depicted in Table I, application no. 17, column 8, in the same line as SEQ ID NO. 9333, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 9333 is depicted, plastidic is mentioned, or a mitochondrial transit peptide in case in Table I, application no. 17, in column 6 in the same line as SEQ ID NO. 9333 is depicted, mitochondric is mentioned; which is operable linked, too.

In another embodiment thereof the expression cassette does not comprise an additional transit peptide in case in Table I, application no. 17, in column 6 in the same line as SEQ ID NO. 9333 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 146327, or a homolog or fragment thereof, or a homolog thereof being depicted in Table I, application no. 17, column 8, in the same line as SEQ ID NO. 146327, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 146327 is depicted, plastidic is mentioned, or a mitochondrial transit peptide in case in Table I, application no. 17, in column 6 in the same line as SEQ ID NO. 146327 is depicted, mitochondric is mentioned; which is operable linked, too.

In another embodiment thereof the expression cassette does not comprise an additional transit peptide in case in Table I, application no. 17, in column 6 in the same line as SEQ ID NO. 146327 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 104660, or a homolog or fragment thereof, or a homolog thereof being depicted in Table I, application no. 17, column 8, in the same line as SEQ ID NO. 104660, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 104660 is depicted, plastidic is mentioned, or a mitochondrial transit peptide in case in Table I, application no. 17, in column 6 in the same line as SEQ ID NO. 104660 is depicted, mitochondric is mentioned; which is operable linked, too.

In another embodiment thereof the expression cassette does not comprise an additional transit peptide in case in Table I, application no. 17, in column 6 in the same line as SEQ ID NO. 104660 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 42477, or a homolog or fragment thereof, or a homolog thereof being depicted in Table I, application no. 17, column 8, in the same line as SEQ ID NO. 42477, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 42477 is depicted, plastidic is mentioned, or a mitochondrial transit peptide in case in Table I, application no. 17, in column 6 in the same line as SEQ ID NO. 42477 is depicted, mitochondric is mentioned; which is operable linked, too.

In another embodiment thereof the expression cassette does not comprise an additional transit peptide in case in Table I, application no. 17, in column 6 in the same line as SEQ ID NO. 42477 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 82510, or a homolog or fragment thereof, or a homolog thereof being depicted in Table I, application no. 17, column 8, in the same line as SEQ ID NO. 82510, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 82510 is depicted, plastidic is mentioned, or a mitochondrial transit peptide in case in Table I, application no. 17, in column 6 in the same line as SEQ ID NO. 82510 is depicted, mitochondric is mentioned; which is operable linked, too.

In another embodiment thereof the expression cassette does not comprise an additional transit peptide in case in Table I, application no. 17, in column 6 in the same line as SEQ ID NO. 82510 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 9470, or a homolog or fragment thereof, or a homolog thereof being depicted in Table I, application no. 17, column 8, in the same line as SEQ ID NO. 9470, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 9470 is depicted, plastidic is mentioned, or a mitochondrial transit peptide in case in Table I, application no. 17, in column 6 in the same line as SEQ ID NO. 9470 is depicted, mitochondric is mentioned; which is operable linked, too.

In another embodiment thereof the expression cassette does not comprise an additional transit peptide in case in Table I, application no. 17, in column 6 in the same line as SEQ ID NO. 9470 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 9492, or a homolog or fragment thereof, or a homolog thereof being depicted in Table I, application no. 17, column 8, in the same line as SEQ ID NO. 9492, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 9492 is depicted, plastidic is mentioned, or a mitochondrial transit peptide in case in Table I, application no. 17, in column 6 in the same line as SEQ ID NO. 9492 is depicted, mitochondric is mentioned; which is operable linked, too.

In another embodiment thereof the expression cassette does not comprise an additional transit peptide in case in Table I, application no. 17, in column 6 in the same line as SEQ ID NO. 9492 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 42502, or a homolog or fragment thereof, or a homolog thereof being depicted in Table I, application no. 17, column 8, in the same line as SEQ ID NO. 42502, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 42502 is depicted, plastidic is mentioned, or a mitochondrial transit peptide in case in Table I, application no. 17, in column 6 in the same line as SEQ ID NO. 42502 is depicted, mitochondric is mentioned; which is operable linked, too.

In another embodiment thereof the expression cassette does not comprise an additional transit peptide in case in Table I, application no. 17, in column 6 in the same line as SEQ ID NO. 42502 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 146364, or a homolog or fragment thereof, or a homolog thereof being depicted in Table I, application no. 17, column 8, in the same line as SEQ ID NO. 146364, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 146364 is depicted, plastidic is mentioned, or a mitochondrial transit peptide in case in Table I, application no. 17, in column 6 in the same line as SEQ ID NO. 146364 is depicted, mitochondric is mentioned; which is operable linked, too.

In another embodiment thereof the expression cassette does not comprise an additional transit peptide in case in Table I, application no. 17, in column 6 in the same line as SEQ ID NO. 146364 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 10104, or a homolog or fragment thereof, or a homolog thereof being depicted in Table I, application no. 17, column 8, in the same line as SEQ ID NO. 10104, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 10104 is depicted, plastidic is mentioned, or a mitochondrial transit peptide in case in Table I, application no. 17, in column 6 in the same line as SEQ ID NO. 10104 is depicted, mitochondric is mentioned; which is operable linked, too.

In another embodiment thereof the expression cassette does not comprise an additional transit peptide in case in Table I, application no. 17, in column 6 in the same line as SEQ ID NO. 10104 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 82866, or a homolog or fragment thereof, or a homolog thereof being depicted in Table I, application no. 17, column 8, in the same line as SEQ ID NO. 82866, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 82866 is depicted, plastidic is mentioned, or a mitochondrial transit peptide in case in Table I, application no. 17, in column 6 in the same line as SEQ ID NO. 82866 is depicted, mitochondric is mentioned; which is operable linked, too.

In another embodiment thereof the expression cassette does not comprise an additional transit peptide in case in Table I, application no. 17, in column 6 in the same line as SEQ ID NO. 82866 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 42579, or a homolog or fragment thereof, or a homolog thereof being depicted in Table I, application no. 17, column 8, in the same line as SEQ ID NO. 42579, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 42579 is depicted, plastidic is mentioned, or a mitochondrial transit peptide in case in Table I, application no. 17, in column 6 in the same line as SEQ ID NO. 42579 is depicted, mitochondric is mentioned; which is operable linked, too.

In another embodiment thereof the expression cassette does not comprise an additional transit peptide in case in Table I, application no. 17, in column 6 in the same line as SEQ ID NO. 42579 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 94051, or a homolog or fragment thereof, or a homolog thereof being depicted in Table I, application no. 17, column 8, in the same line as SEQ ID NO. 94051, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 94051 is depicted, plastidic is mentioned, or a mitochondrial transit peptide in case in Table I, application no. 17, in column 6 in the same line as SEQ ID NO. 94051 is depicted, mitochondric is mentioned; which is operable linked, too.

In another embodiment thereof the expression cassette does not comprise an additional transit peptide in case in Table I, application no. 17, in column 6 in the same line as SEQ ID NO. 94051 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 94380, or a homolog or fragment thereof, or a homolog thereof being depicted in Table I, application no. 17, column 8, in the same line as SEQ ID NO. 94380, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 94380 is depicted, plastidic is mentioned, or a mitochondrial transit peptide in case in Table I, application no. 17, in column 6 in the same line as SEQ ID NO. 94380 is depicted, mitochondric is mentioned; which is operable linked, too.

In another embodiment thereof the expression cassette does not comprise an additional transit peptide in case in Table I, application no. 17, in column 6 in the same line as SEQ ID NO. 94380 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 146440, or a homolog or fragment thereof, or a homolog thereof being depicted in Table I, application no. 17, column 8, in the same line as SEQ ID NO. 146440, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 146440 is depicted, plastidic is mentioned, or a mitochondrial transit peptide in case in Table I, application no. 17, in column 6 in the same line as SEQ ID NO. 146440 is depicted, mitochondric is mentioned; which is operable linked, too.

In another embodiment thereof the expression cassette does not comprise an additional transit peptide in case in Table I, application no. 17, in column 6 in the same line as SEQ ID NO. 146440 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 116172, or a homolog or fragment thereof, or a homolog thereof being depicted in Table I, application no. 17, column 8, in the same line as SEQ ID NO. 116172, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 116172 is depicted, plastidic is mentioned, or a mitochondrial transit peptide in case in Table I, application no. 17, in column 6 in the same line as SEQ ID NO. 116172 is depicted, mitochondric is mentioned; which is operable linked, too.

In another embodiment thereof the expression cassette does not comprise an additional transit peptide in case in Table I, application no. 17, in column 6 in the same line as SEQ ID NO. 116172 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 146472, or a homolog or fragment thereof, or a homolog thereof being depicted in Table I, application no. 17, column 8, in the same line as SEQ ID NO. 146472, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 146472 is depicted, plastidic is mentioned, or a mitochondrial transit peptide in case in Table I, application no. 17, in column 6 in the same line as SEQ ID NO. 146472 is depicted, mitochondric is mentioned; which is operable linked, too.

In another embodiment thereof the expression cassette does not comprise an additional transit peptide in case in Table I, application no. 17, in column 6 in the same line as SEQ ID NO. 146472 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 146646, or a homolog or fragment thereof, or a homolog thereof being depicted in Table I, application no. 17, column 8, in the same line as SEQ ID NO. 146646, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 146646 is depicted, plastidic is mentioned, or a mitochondrial transit peptide in case in Table I, application no. 17, in column 6 in the same line as SEQ ID NO. 146646 is depicted, mitochondric is mentioned; which is operable linked, too.

In another embodiment thereof the expression cassette does not comprise an additional transit peptide in case in Table I, application no. 17, in column 6 in the same line as SEQ ID NO. 146646 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 146732, or a homolog or fragment thereof, or a homolog thereof being depicted in Table I, application no. 17, column 8, in the same line as SEQ ID NO. 146732, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 146732 is depicted, plastidic is mentioned, or a mitochondrial transit peptide in case in Table I, application no. 17, in column 6 in the same line as SEQ ID NO. 146732 is depicted, mitochondric is mentioned; which is operable linked, too.

In another embodiment thereof the expression cassette does not comprise an additional transit peptide in case in Table I, application no. 17, in column 6 in the same line as SEQ ID NO. 146732 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 94386, or a homolog or fragment thereof, or a homolog thereof being depicted in Table I, application no. 17, column 8, in the same line as SEQ ID NO. 94386, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 94386 is depicted, plastidic is mentioned, or a mitochondrial transit peptide in case in Table I, application no. 17, in column 6 in the same line as SEQ ID NO. 94386 is depicted, mitochondric is mentioned; which is operable linked, too.

In another embodiment thereof the expression cassette does not comprise an additional transit peptide in case in Table I, application no. 17, in column 6 in the same line as SEQ ID NO. 94386 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 136098, or a homolog or fragment thereof, or a homolog thereof being depicted in Table I, application no. 17, column 8, in the same line as SEQ ID NO. 136098, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 136098 is depicted, plastidic is mentioned, or a mitochondrial transit peptide in case in Table I, application no. 17, in column 6 in the same line as SEQ ID NO. 136098 is depicted, mitochondric is mentioned; which is operable linked, too.

In another embodiment thereof the expression cassette does not comprise an additional transit peptide in case in Table I, application no. 17, in column 6 in the same line as SEQ ID NO. 136098 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 10708, or a homolog or fragment thereof, or a homolog thereof being depicted in Table I, application no. 17, column 8, in the same line as SEQ ID NO. 10708, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 10708 is depicted, plastidic is mentioned, or a mitochondrial transit peptide in case in Table I, application no. 17, in column 6 in the same line as SEQ ID NO. 10708 is depicted, mitochondric is mentioned; which is operable linked, too.

In another embodiment thereof the expression cassette does not comprise an additional transit peptide in case in Table I, application no. 17, in column 6 in the same line as SEQ ID NO. 10708 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 146834, or a homolog or fragment thereof, or a homolog thereof being depicted in Table I, application no. 17, column 8, in the same line as SEQ ID NO. 146834, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 146834 is depicted, plastidic is mentioned, or a mitochondrial transit peptide in case in Table I, application no. 17, in column 6 in the same line as SEQ ID NO. 146834 is depicted, mitochondric is mentioned; which is operable linked, too.

In another embodiment thereof the expression cassette does not comprise an additional transit peptide in case in Table I, application no. 17, in column 6 in the same line as SEQ ID NO. 146834 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 10726, or a homolog or fragment thereof, or a homolog thereof being depicted in Table I, application no. 17, column 8, in the same line as SEQ ID NO. 10726, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 10726 is depicted, plastidic is mentioned, or a mitochondrial transit peptide in case in Table I, application no. 17, in column 6 in the same line as SEQ ID NO. 10726 is depicted, mitochondric is mentioned; which is operable linked, too.

In another embodiment thereof the expression cassette does not comprise an additional transit peptide in case in Table I, application no. 17, in column 6 in the same line as SEQ ID NO. 10726 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 101327, or a homolog or fragment thereof, or a homolog thereof being depicted in Table I, application no. 17, column 8, in the same line as SEQ ID NO. 101327, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 101327 is depicted, plastidic is mentioned, or a mitochondrial transit peptide in case in Table I, application no. 17, in column 6 in the same line as SEQ ID NO. 101327 is depicted, mitochondric is mentioned; which is operable linked, too.

In another embodiment thereof the expression cassette does not comprise an additional transit peptide in case in Table I, application no. 17, in column 6 in the same line as SEQ ID NO. 101327 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 83821, or a homolog or fragment thereof, or a homolog thereof being depicted in Table I, application no. 17, column 8, in the same line as SEQ ID NO. 83821, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 83821 is depicted, plastidic is mentioned, or a mitochondrial transit peptide in case in Table I, application no. 17, in column 6 in the same line as SEQ ID NO. 83821 is depicted, mitochondric is mentioned; which is operable linked, too.

In another embodiment thereof the expression cassette does not comprise an additional transit peptide in case in Table I, application no. 17, in column 6 in the same line as SEQ ID NO. 83821 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 44372, or a homolog or fragment thereof, or a homolog thereof being depicted in Table I, application no. 17, column 8, in the same line as SEQ ID NO. 44372, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 44372 is depicted, plastidic is mentioned, or a mitochondrial transit peptide in case in Table I, application no. 17, in column 6 in the same line as SEQ ID NO. 44372 is depicted, mitochondric is mentioned; which is operable linked, too.

In another embodiment thereof the expression cassette does not comprise an additional transit peptide in case in Table I, application no. 17, in column 6 in the same line as SEQ ID NO. 44372 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 44378, or a homolog or fragment thereof, or a homolog thereof being depicted in Table I, application no. 17, column 8, in the same line as SEQ ID NO. 44378, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 44378 is depicted, plastidic is mentioned, or a mitochondrial transit peptide in case in Table I, application no. 17, in column 6 in the same line as SEQ ID NO. 44378 is depicted, mitochondric is mentioned; which is operable linked, too.

In another embodiment thereof the expression cassette does not comprise an additional transit peptide in case in Table I, application no. 17, in column 6 in the same line as SEQ ID NO. 44378 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 10740, or a homolog or fragment thereof, or a homolog thereof being depicted in Table I, application no. 17, column 8, in the same line as SEQ ID NO. 10740, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 10740 is depicted, plastidic is mentioned, or a mitochondrial transit peptide in case in Table I, application no. 17, in column 6 in the same line as SEQ ID NO. 10740 is depicted, mitochondric is mentioned; which is operable linked, too.

In another embodiment thereof the expression cassette does not comprise an additional transit peptide in case in Table I, application no. 17, in column 6 in the same line as SEQ ID NO. 10740 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 146848, or a homolog or fragment thereof, or a homolog thereof being depicted in Table I, application no. 17, column 8, in the same line as SEQ ID NO. 146848, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 146848 is depicted, plastidic is mentioned, or a mitochondrial transit peptide in case in Table I, application no. 17, in column 6 in the same line as SEQ ID NO. 146848 is depicted, mitochondric is mentioned; which is operable linked, too.

In another embodiment thereof the expression cassette does not comprise an additional transit peptide in case in Table I, application no. 17, in column 6 in the same line as SEQ ID NO. 146848 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 106532, or a homolog or fragment thereof, or a homolog thereof being depicted in Table I, application no. 17, column 8, in the same line as SEQ ID NO. 106532, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 106532 is depicted, plastidic is mentioned, or a mitochondrial transit peptide in case in Table I, application no. 17, in column 6 in the same line as SEQ ID NO. 106532 is depicted, mitochondric is mentioned; which is operable linked, too.

In another embodiment thereof the expression cassette does not comprise an additional transit peptide in case in Table I, application no. 17, in column 6 in the same line as SEQ ID NO. 106532 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 94542, or a homolog or fragment thereof, or a homolog thereof being depicted in Table I, application no. 17, column 8, in the same line as SEQ ID NO. 94542, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 94542 is depicted, plastidic is mentioned, or a mitochondrial transit peptide in case in Table I, application no. 17, in column 6 in the same line as SEQ ID NO. 94542 is depicted, mitochondric is mentioned; which is operable linked, too.

In another embodiment thereof the expression cassette does not comprise an additional transit peptide in case in Table I, application no. 17, in column 6 in the same line as SEQ ID NO. 94542 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 126421, or a homolog or fragment thereof, or a homolog thereof being depicted in Table I, application no. 17, column 8, in the same line as SEQ ID NO. 126421, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 126421 is depicted, plastidic is mentioned, or a mitochondrial transit peptide in case in Table I, application no. 17, in column 6 in the same line as SEQ ID NO. 126421 is depicted, mitochondric is mentioned; which is operable linked, too.

In another embodiment thereof the expression cassette does not comprise an additional transit peptide in case in Table I, application no. 17, in column 6 in the same line as SEQ ID NO. 126421 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 146921, or a homolog or fragment thereof, or a homolog thereof being depicted in Table I, application no. 17, column 8, in the same line as SEQ ID NO. 146921, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 146921 is depicted, plastidic is mentioned, or a mitochondrial transit peptide in case in Table I, application no. 17, in column 6 in the same line as SEQ ID NO. 146921 is depicted, mitochondric is mentioned; which is operable linked, too.

In another embodiment thereof the expression cassette does not comprise an additional transit peptide in case in Table I, application no. 17, in column 6 in the same line as SEQ ID NO. 146921 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 94735, or a homolog or fragment thereof, or a homolog thereof being depicted in Table I, application no. 17, column 8, in the same line as SEQ ID NO. 94735, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 94735 is depicted, plastidic is mentioned, or a mitochondrial transit peptide in case in Table I, application no. 17, in column 6 in the same line as SEQ ID NO. 94735 is depicted, mitochondric is mentioned; which is operable linked, too.

In another embodiment thereof the expression cassette does not comprise an additional transit peptide in case in Table I, application no. 17, in column 6 in the same line as SEQ ID NO. 94735 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 45321, or a homolog or fragment thereof, or a homolog thereof being depicted in Table I, application no. 17, column 8, in the same line as SEQ ID NO. 45321, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 45321 is depicted, plastidic is mentioned, or a mitochondrial transit peptide in case in Table I, application no. 17, in column 6 in the same line as SEQ ID NO. 45321 is depicted, mitochondric is mentioned; which is operable linked, too.

In another embodiment thereof the expression cassette does not comprise an additional transit peptide in case in Table I, application no. 17, in column 6 in the same line as SEQ ID NO. 45321 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 45394, or a homolog or fragment thereof, or a homolog thereof being depicted in Table I, application no. 17, column 8, in the same line as SEQ ID NO. 45394, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 45394 is depicted, plastidic is mentioned, or a mitochondrial transit peptide in case in Table I, application no. 17, in column 6 in the same line as SEQ ID NO. 45394 is depicted, mitochondric is mentioned; which is operable linked, too.

In another embodiment thereof the expression cassette does not comprise an additional transit peptide in case in Table I, application no. 17, in column 6 in the same line as SEQ ID NO. 45394 is depicted, non-targeted is mentioned.

In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 45556, or a homolog or fragment thereof, or a homolog thereof being depicted in Table I, application no. 17, column 8, in the same line as SEQ ID NO. 45556, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 45556 is depicted, plastidic is mentioned, or a mitochondrial transit peptide in case in Table I, application no. 17, in column 6 in the same line as SEQ ID NO. 45556 is depicted, mitochondric is mentioned; which is operable linked, too.

In another embodiment thereof the expression cassette does not comprise an additional transit peptide in case in Table I, application no. 17, in column 6 in the same line as SEQ ID NO. 45556 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 84079, or a homolog or fragment thereof, or a homolog thereof being depicted in Table I, application no. 17, column 8, in the same line as SEQ ID NO. 84079, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 84079 is depicted, plastidic is mentioned, or a mitochondrial transit peptide in case in Table I, application no. 17, in column 6 in the same line as SEQ ID NO. 84079 is depicted, mitochondric is mentioned; which is operable linked, too.

In another embodiment thereof the expression cassette does not comprise an additional transit peptide in case in Table I, application no. 17, in column 6 in the same line as SEQ ID NO. 84079 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 10811, or a homolog or fragment thereof, or a homolog thereof being depicted in Table I, application no. 17, column 8, in the same line as SEQ ID NO. 10811, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 10811 is depicted, plastidic is mentioned, or a mitochondrial transit peptide in case in Table I, application no. 17, in column 6 in the same line as SEQ ID NO. 10811 is depicted, mitochondric is mentioned; which is operable linked, too.

In another embodiment thereof the expression cassette does not comprise an additional transit peptide in case in Table I, application no. 17, in column 6 in the same line as SEQ ID NO. 10811 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 46751, or a homolog or fragment thereof, or a homolog thereof being depicted in Table I, application no. 17, column 8, in the same line as SEQ ID NO. 46751, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 46751 is depicted, plastidic is mentioned, or a mitochondrial transit peptide in case in Table I, application no. 17, in column 6 in the same line as SEQ ID NO. 46751 is depicted, mitochondric is mentioned; which is operable linked, too.

In another embodiment thereof the expression cassette does not comprise an additional transit peptide in case in Table I, application no. 17, in column 6 in the same line as SEQ ID NO. 46751 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 84198, or a homolog or fragment thereof, or a homolog thereof being depicted in Table I, application no. 17, column 8, in the same line as SEQ ID NO. 84198, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 84198 is depicted, plastidic is mentioned, or a mitochondrial transit peptide in case in Table I, application no. 17, in column 6 in the same line as SEQ ID NO. 84198 is depicted, mitochondric is mentioned; which is operable linked, too.

In another embodiment thereof the expression cassette does not comprise an additional transit peptide in case in Table I, application no. 17, in column 6 in the same line as SEQ ID NO. 84198 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 116312, or a homolog or fragment thereof, or a homolog thereof being depicted in Table I, application no. 17, column 8, in the same line as SEQ ID NO. 116312, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 116312 is depicted, plastidic is mentioned, or a mitochondrial transit peptide in case in Table I, application no. 17, in column 6 in the same line as SEQ ID NO. 116312 is depicted, mitochondric is mentioned; which is operable linked, too.

In another embodiment thereof the expression cassette does not comprise an additional transit peptide in case in Table I, application no. 17, in column 6 in the same line as SEQ ID NO. 116312 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 46850, or a homolog or fragment thereof, or a homolog thereof being depicted in Table I, application no. 17, column 8, in the same line as SEQ ID NO. 46850, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 46850 is depicted, plastidic is mentioned, or a mitochondrial transit peptide in case in Table I, application no. 17, in column 6 in the same line as SEQ ID NO. 46850 is depicted, mitochondric is mentioned; which is operable linked, too.

In another embodiment thereof the expression cassette does not comprise an additional transit peptide in case in Table I, application no. 17, in column 6 in the same line as SEQ ID NO. 46850 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 47026, or a homolog or fragment thereof, or a homolog thereof being depicted in Table I, application no. 17, column 8, in the same line as SEQ ID NO. 47026, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 47026 is depicted, plastidic is mentioned, or a mitochondrial transit peptide in case in Table I, application no. 17, in column 6 in the same line as SEQ ID NO. 47026 is depicted, mitochondric is mentioned; which is operable linked, too.

In another embodiment thereof the expression cassette does not comprise an additional transit peptide in case in Table I, application no. 17, in column 6 in the same line as SEQ ID NO. 47026 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 47105, or a homolog or fragment thereof, or a homolog thereof being depicted in Table I, application no. 17, column 8, in the same line as SEQ ID NO. 47105, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 47105 is depicted, plastidic is mentioned, or a mitochondrial transit peptide in case in Table I, application no. 17, in column 6 in the same line as SEQ ID NO. 47105 is depicted, mitochondric is mentioned; which is operable linked, too.

In another embodiment thereof the expression cassette does not comprise an additional transit peptide in case in Table I, application no. 17, in column 6 in the same line as SEQ ID NO. 47105 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 146939, or a homolog or fragment thereof, or a homolog thereof being depicted in Table I, application no. 17, column 8, in the same line as SEQ ID NO. 146939, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 146939 is depicted, plastidic is mentioned, or a mitochondrial transit peptide in case in Table I, application no. 17, in column 6 in the same line as SEQ ID NO. 146939 is depicted, mitochondric is mentioned; which is operable linked, too.

In another embodiment thereof the expression cassette does not comprise an additional transit peptide in case in Table I, application no. 17, in column 6 in the same line as SEQ ID NO. 146939 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 49143, or a homolog or fragment thereof, or a homolog thereof being depicted in Table I, application no. 17, column 8, in the same line as SEQ ID NO. 49143, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 49143 is depicted, plastidic is mentioned, or a mitochondrial transit peptide in case in Table I, application no. 17, in column 6 in the same line as SEQ ID NO. 49143 is depicted, mitochondric is mentioned; which is operable linked, too.

In another embodiment thereof the expression cassette does not comprise an additional transit peptide in case in Table I, application no. 17, in column 6 in the same line as SEQ ID NO. 49143 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 49800, or a homolog or fragment thereof, or a homolog thereof being depicted in Table I, application no. 17, column 8, in the same line as SEQ ID NO. 49800, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 49800 is depicted, plastidic is mentioned, or a mitochondrial transit peptide in case in Table I, application no. 17, in column 6 in the same line as SEQ ID NO. 49800 is depicted, mitochondric is mentioned; which is operable linked, too.

In another embodiment thereof the expression cassette does not comprise an additional transit peptide in case in Table I, application no. 17, in column 6 in the same line as SEQ ID NO. 49800 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 84865, or a homolog or fragment thereof, or a homolog thereof being depicted in Table I, application no. 17, column 8, in the same line as SEQ ID NO. 84865, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 84865 is depicted, plastidic is mentioned, or a mitochondrial transit peptide in case in Table I, application no. 17, in column 6 in the same line as SEQ ID NO. 84865 is depicted, mitochondric is mentioned; which is operable linked, too.

In another embodiment thereof the expression cassette does not comprise an additional transit peptide in case in Table I, application no. 17, in column 6 in the same line as SEQ ID NO. 84865 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 50070, or a homolog or fragment thereof, or a homolog thereof being depicted in Table I, application no. 17, column 8, in the same line as SEQ ID NO. 50070, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 50070 is depicted, plastidic is mentioned, or a mitochondrial transit peptide in case in Table I, application no. 17, in column 6 in the same line as SEQ ID NO. 50070 is depicted, mitochondric is mentioned; which is operable linked, too.

In another embodiment thereof the expression cassette does not comprise an additional transit peptide in case in Table I, application no. 17, in column 6 in the same line as SEQ ID NO. 50070 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 106920, or a homolog or fragment thereof, or a homolog thereof being depicted in Table I, application no. 17, column 8, in the same line as SEQ ID NO. 106920, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 106920 is depicted, plastidic is mentioned, or a mitochondrial transit peptide in case in Table I, application no. 17, in column 6 in the same line as SEQ ID NO. 106920 is depicted, mitochondric is mentioned; which is operable linked, too.

In another embodiment thereof the expression cassette does not comprise an additional transit peptide in case in Table I, application no. 17, in column 6 in the same line as SEQ ID NO. 106920 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 94942, or a homolog or fragment thereof, or a homolog thereof being depicted in Table I, application no. 17, column 8, in the same line as SEQ ID NO. 94942, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 94942 is depicted, plastidic is mentioned, or a mitochondrial transit peptide in case in Table I, application no. 17, in column 6 in the same line as SEQ ID NO. 94942 is depicted, mitochondric is mentioned; which is operable linked, too.

In another embodiment thereof the expression cassette does not comprise an additional transit peptide in case in Table I, application no. 17, in column 6 in the same line as SEQ ID NO. 94942 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 50104, or a homolog or fragment thereof, or a homolog thereof being depicted in Table I, application no. 17, column 8, in the same line as SEQ ID NO. 50104, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 50104 is depicted, plastidic is mentioned, or a mitochondrial transit peptide in case in Table I, application no. 17, in column 6 in the same line as SEQ ID NO. 50104 is depicted, mitochondric is mentioned; which is operable linked, too.

In another embodiment thereof the expression cassette does not comprise an additional transit peptide in case in Table I, application no. 17, in column 6 in the same line as SEQ ID NO. 50104 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 85510, or a homolog or fragment thereof, or a homolog thereof being depicted in Table I, application no. 17, column 8, in the same line as SEQ ID NO. 85510, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 85510 is depicted, plastidic is mentioned, or a mitochondrial transit peptide in case in Table I, application no. 17, in column 6 in the same line as SEQ ID NO. 85510 is depicted, mitochondric is mentioned; which is operable linked, too.

In another embodiment thereof the expression cassette does not comprise an additional transit peptide in case in Table I, application no. 17, in column 6 in the same line as SEQ ID NO. 85510 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 146945, or a homolog or fragment thereof, or a homolog thereof being depicted in Table I, application no. 17, column 8, in the same line as SEQ ID NO. 146945, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 146945 is depicted, plastidic is mentioned, or a mitochondrial transit peptide in case in Table I, application no. 17, in column 6 in the same line as SEQ ID NO. 146945 is depicted, mitochondric is mentioned; which is operable linked, too.

In another embodiment thereof the expression cassette does not comprise an additional transit peptide in case in Table I, application no. 17, in column 6 in the same line as SEQ ID NO. 146945 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 101707, or a homolog or fragment thereof, or a homolog thereof being depicted in Table I, application no. 17, column 8, in the same line as SEQ ID NO. 101707, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 101707 is depicted, plastidic is mentioned, or a mitochondrial transit peptide in case in Table I, application no. 17, in column 6 in the same line as SEQ ID NO. 101707 is depicted, mitochondric is mentioned; which is operable linked, too.

In another embodiment thereof the expression cassette does not comprise an additional transit peptide in case in Table I, application no. 17, in column 6 in the same line as SEQ ID NO. 101707 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 52246, or a homolog or fragment thereof, or a homolog thereof being depicted in Table I, application no. 17, column 8, in the same line as SEQ ID NO. 52246, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 52246 is depicted, plastidic is mentioned, or a mitochondrial transit peptide in case in Table I, application no. 17, in column 6 in the same line as SEQ ID NO. 52246 is depicted, mitochondric is mentioned; which is operable linked, too.

In another embodiment thereof the expression cassette does not comprise an additional transit peptide in case in Table I, application no. 17, in column 6 in the same line as SEQ ID NO. 52246 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 52364, or a homolog or fragment thereof, or a homolog thereof being depicted in Table I, application no. 17, column 8, in the same line as SEQ ID NO. 52364, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 52364 is depicted, plastidic is mentioned, or a mitochondrial transit peptide in case in Table I, application no. 17, in column 6 in the same line as SEQ ID NO. 52364 is depicted, mitochondric is mentioned; which is operable linked, too.

In another embodiment thereof the expression cassette does not comprise an additional transit peptide in case in Table I, application no. 17, in column 6 in the same line as SEQ ID NO. 52364 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 53189, or a homolog or fragment thereof, or a homolog thereof being depicted in Table I, application no. 17, column 8, in the same line as SEQ ID NO. 53189, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 53189 is depicted, plastidic is mentioned, or a mitochondrial transit peptide in case in Table I, application no. 17, in column 6 in the same line as SEQ ID NO. 53189 is depicted, mitochondric is mentioned; which is operable linked, too.

In another embodiment thereof the expression cassette does not comprise an additional transit peptide in case in Table I, application no. 17, in column 6 in the same line as SEQ ID NO. 53189 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 147208, or a homolog or fragment thereof, or a homolog thereof being depicted in Table I, application no. 17, column 8, in the same line as SEQ ID NO. 147208, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 147208 is depicted, plastidic is mentioned, or a mitochondrial transit peptide in case in Table I, application no. 17, in column 6 in the same line as SEQ ID NO. 147208 is depicted, mitochondric is mentioned; which is operable linked, too.

In another embodiment thereof the expression cassette does not comprise an additional transit peptide in case in Table I, application no. 17, in column 6 in the same line as SEQ ID NO. 147208 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 53456, or a homolog or fragment thereof, or a homolog thereof being depicted in Table I, application no. 17, column 8, in the same line as SEQ ID NO. 53456, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 53456 is depicted, plastidic is mentioned, or a mitochondrial transit peptide in case in Table I, application no. 17, in column 6 in the same line as SEQ ID NO. 53456 is depicted, mitochondric is mentioned; which is operable linked, too.

In another embodiment thereof the expression cassette does not comprise an additional transit peptide in case in Table I, application no. 17, in column 6 in the same line as SEQ ID NO. 53456 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 95047, or a homolog or fragment thereof, or a homolog thereof being depicted in Table I, application no. 17, column 8, in the same line as SEQ ID NO. 95047, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 95047 is depicted, plastidic is mentioned, or a mitochondrial transit peptide in case in Table I, application no. 17, in column 6 in the same line as SEQ ID NO. 95047 is depicted, mitochondric is mentioned; which is operable linked, too.

In another embodiment thereof the expression cassette does not comprise an additional transit peptide in case in Table I, application no. 17, in column 6 in the same line as SEQ ID NO. 95047 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 54897, or a homolog or fragment thereof, or a homolog thereof being depicted in Table I, application no. 17, column 8, in the same line as SEQ ID NO. 54897, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 54897 is depicted, plastidic is mentioned, or a mitochondrial transit peptide in case in Table I, application no. 17, in column 6 in the same line as SEQ ID NO. 54897 is depicted, mitochondric is mentioned; which is operable linked, too.

In another embodiment thereof the expression cassette does not comprise an additional transit peptide in case in Table I, application no. 17, in column 6 in the same line as SEQ ID NO. 54897 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 55063, or a homolog or fragment thereof, or a homolog thereof being depicted in Table I, application no. 17, column 8, in the same line as SEQ ID NO. 55063, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 55063 is depicted, plastidic is mentioned, or a mitochondrial transit peptide in case in Table I, application no. 17, in column 6 in the same line as SEQ ID NO. 55063 is depicted, mitochondric is mentioned; which is operable linked, too.

In another embodiment thereof the expression cassette does not comprise an additional transit peptide in case in Table I, application no. 17, in column 6 in the same line as SEQ ID NO. 55063 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 55379, or a homolog or fragment thereof, or a homolog thereof being depicted in Table I, application no. 17, column 8, in the same line as SEQ ID NO. 55379, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 55379 is depicted, plastidic is mentioned, or a mitochondrial transit peptide in case in Table I, application no. 17, in column 6 in the same line as SEQ ID NO. 55379 is depicted, mitochondric is mentioned; which is operable linked, too.

In another embodiment thereof the expression cassette does not comprise an additional transit peptide in case in Table I, application no. 17, in column 6 in the same line as SEQ ID NO. 55379 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 86447, or a homolog or fragment thereof, or a homolog thereof being depicted in Table I, application no. 17, column 8, in the same line as SEQ ID NO. 86447, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 86447 is depicted, plastidic is mentioned, or a mitochondrial transit peptide in case in Table I, application no. 17, in column 6 in the same line as SEQ ID NO. 86447 is depicted, mitochondric is mentioned; which is operable linked, too.

In another embodiment thereof the expression cassette does not comprise an additional transit peptide in case in Table I, application no. 17, in column 6 in the same line as SEQ ID NO. 86447 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 55385, or a homolog or fragment thereof, or a homolog thereof being depicted in Table I, application no. 17, column 8, in the same line as SEQ ID NO. 55385, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 55385 is depicted, plastidic is mentioned, or a mitochondrial transit peptide in case in Table I, application no. 17, in column 6 in the same line as SEQ ID NO. 55385 is depicted, mitochondric is mentioned; which is operable linked, too.

In another embodiment thereof the expression cassette does not comprise an additional transit peptide in case in Table I, application no. 17, in column 6 in the same line as SEQ ID NO. 55385 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 55771, or a homolog or fragment thereof, or a homolog thereof being depicted in Table I, application no. 17, column 8, in the same line as SEQ ID NO. 55771, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 55771 is depicted, plastidic is mentioned, or a mitochondrial transit peptide in case in Table I, application no. 17, in column 6 in the same line as SEQ ID NO. 55771 is depicted, mitochondric is mentioned; which is operable linked, too.

In another embodiment thereof the expression cassette does not comprise an additional transit peptide in case in Table I, application no. 17, in column 6 in the same line as SEQ ID NO. 55771 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 86540, or a homolog or fragment thereof, or a homolog thereof being depicted in Table I, application no. 17, column 8, in the same line as SEQ ID NO. 86540, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 86540 is depicted, plastidic is mentioned, or a mitochondrial transit peptide in case in Table I, application no. 17, in column 6 in the same line as SEQ ID NO. 86540 is depicted, mitochondric is mentioned; which is operable linked, too.

In another embodiment thereof the expression cassette does not comprise an additional transit peptide in case in Table I, application no. 17, in column 6 in the same line as SEQ ID NO. 86540 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 11990, or a homolog or fragment thereof, or a homolog thereof being depicted in Table I, application no. 17, column 8, in the same line as SEQ ID NO. 11990, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 11990 is depicted, plastidic is mentioned, or a mitochondrial transit peptide in case in Table I, application no. 17, in column 6 in the same line as SEQ ID NO. 11990 is depicted, mitochondric is mentioned; which is operable linked, too.

In another embodiment thereof the expression cassette does not comprise an additional transit peptide in case in Table I, application no. 17, in column 6 in the same line as SEQ ID NO. 11990 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 56514, or a homolog or fragment thereof, or a homolog thereof being depicted in Table I, application no. 17, column 8, in the same line as SEQ ID NO. 56514, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 56514 is depicted, plastidic is mentioned, or a mitochondrial transit peptide in case in Table I, application no. 17, in column 6 in the same line as SEQ ID NO. 56514 is depicted, mitochondric is mentioned; which is operable linked, too.

In another embodiment thereof the expression cassette does not comprise an additional transit peptide in case in Table I, application no. 17, in column 6 in the same line as SEQ ID NO. 56514 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 56894, or a homolog or fragment thereof, or a homolog thereof being depicted in Table I, application no. 17, column 8, in the same line as SEQ ID NO. 56894, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 56894 is depicted, plastidic is mentioned, or a mitochondrial transit peptide in case in Table I, application no. 17, in column 6 in the same line as SEQ ID NO. 56894 is depicted, mitochondric is mentioned; which is operable linked, too.

In another embodiment thereof the expression cassette does not comprise an additional transit peptide in case in Table I, application no. 17, in column 6 in the same line as SEQ ID NO. 56894 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 87397, or a homolog or fragment thereof, or a homolog thereof being depicted in Table I, application no. 17, column 8, in the same line as SEQ ID NO. 87397, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 87397 is depicted, plastidic is mentioned, or a mitochondrial transit peptide in case in Table I, application no. 17, in column 6 in the same line as SEQ ID NO. 87397 is depicted, mitochondric is mentioned; which is operable linked, too.

In another embodiment thereof the expression cassette does not comprise an additional transit peptide in case in Table I, application no. 17, in column 6 in the same line as SEQ ID NO. 87397 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 57235, or a homolog or fragment thereof, or a homolog thereof being depicted in Table I, application no. 17, column 8, in the same line as SEQ ID NO. 57235, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 57235 is depicted, plastidic is mentioned, or a mitochondrial transit peptide in case in Table I, application no. 17, in column 6 in the same line as SEQ ID NO. 57235 is depicted, mitochondric is mentioned; which is operable linked, too.

In another embodiment thereof the expression cassette does not comprise an additional transit peptide in case in Table I, application no. 17, in column 6 in the same line as SEQ ID NO. 57235 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 147346, or a homolog or fragment thereof, or a homolog thereof being depicted in Table I, application no. 17, column 8, in the same line as SEQ ID NO. 147346, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 147346 is depicted, plastidic is mentioned, or a mitochondrial transit peptide in case in Table I, application no. 17, in column 6 in the same line as SEQ ID NO. 147346 is depicted, mitochondric is mentioned; which is operable linked, too.

In another embodiment thereof the expression cassette does not comprise an additional transit peptide in case in Table I, application no. 17, in column 6 in the same line as SEQ ID NO. 147346 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 57679, or a homolog or fragment thereof, or a homolog thereof being depicted in Table I, application no. 17, column 8, in the same line as SEQ ID NO. 57679, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 57679 is depicted, plastidic is mentioned, or a mitochondrial transit peptide in case in Table I, application no. 17, in column 6 in the same line as SEQ ID NO. 57679 is depicted, mitochondric is mentioned; which is operable linked, too.

In another embodiment thereof the expression cassette does not comprise an additional transit peptide in case in Table I, application no. 17, in column 6 in the same line as SEQ ID NO. 57679 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 87610, or a homolog or fragment thereof, or a homolog thereof being depicted in Table I, application no. 17, column 8, in the same line as SEQ ID NO. 87610, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 87610 is depicted, plastidic is mentioned, or a mitochondrial transit peptide in case in Table I, application no. 17, in column 6 in the same line as SEQ ID NO. 87610 is depicted, mitochondric is mentioned; which is operable linked, too.

In another embodiment thereof the expression cassette does not comprise an additional transit peptide in case in Table I, application no. 17, in column 6 in the same line as SEQ ID NO. 87610 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 147407, or a homolog or fragment thereof, or a homolog thereof being depicted in Table I, application no. 17, column 8, in the same line as SEQ ID NO. 147407, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 147407 is depicted, plastidic is mentioned, or a mitochondrial transit peptide in case in Table I, application no. 17, in column 6 in the same line as SEQ ID NO. 147407 is depicted, mitochondric is mentioned; which is operable linked, too.

In another embodiment thereof the expression cassette does not comprise an additional transit peptide in case in Table I, application no. 17, in column 6 in the same line as SEQ ID NO. 147407 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 119222, or a homolog or fragment thereof, or a homolog thereof being depicted in Table I, application no. 17, column 8, in the same line as SEQ ID NO. 119222, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 119222 is depicted, plastidic is mentioned, or a mitochondrial transit peptide in case in Table I, application no. 17, in column 6 in the same line as SEQ ID NO. 119222 is depicted, mitochondric is mentioned; which is operable linked, too.

In another embodiment thereof the expression cassette does not comprise an additional transit peptide in case in Table I, application no. 17, in column 6 in the same line as SEQ ID NO. 119222 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 57734, or a homolog or fragment thereof, or a homolog thereof being depicted in Table I, application no. 17, column 8, in the same line as SEQ ID NO. 57734, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 57734 is depicted, plastidic is mentioned, or a mitochondrial transit peptide in case in Table I, application no. 17, in column 6 in the same line as SEQ ID NO. 57734 is depicted, mitochondric is mentioned; which is operable linked, too.

In another embodiment thereof the expression cassette does not comprise an additional transit peptide in case in Table I, application no. 17, in column 6 in the same line as SEQ ID NO. 57734 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 101776, or a homolog or fragment thereof, or a homolog thereof being depicted in Table I, application no. 17, column 8, in the same line as SEQ ID NO. 101776, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 101776 is depicted, plastidic is mentioned, or a mitochondrial transit peptide in case in Table I, application no. 17, in column 6 in the same line as SEQ ID NO. 101776 is depicted, mitochondric is mentioned; which is operable linked, too.

In another embodiment thereof the expression cassette does not comprise an additional transit peptide in case in Table I, application no. 17, in column 6 in the same line as SEQ ID NO. 101776 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 116713, or a homolog or fragment thereof, or a homolog thereof being depicted in Table I, application no. 17, column 8, in the same line as SEQ ID NO. 116713, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 116713 is depicted, plastidic is mentioned, or a mitochondrial transit peptide in case in Table I, application no. 17, in column 6 in the same line as SEQ ID NO. 116713 is depicted, mitochondric is mentioned; which is operable linked, too.

In another embodiment thereof the expression cassette does not comprise an additional transit peptide in case in Table I, application no. 17, in column 6 in the same line as SEQ ID NO. 116713 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 58472, or a homolog or fragment thereof, or a homolog thereof being depicted in Table I, application no. 17, column 8, in the same line as SEQ ID NO. 58472, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 58472 is depicted, plastidic is mentioned, or a mitochondrial transit peptide in case in Table I, application no. 17, in column 6 in the same line as SEQ ID NO. 58472 is depicted, mitochondric is mentioned; which is operable linked, too.

In another embodiment thereof the expression cassette does not comprise an additional transit peptide in case in Table I, application no. 17, in column 6 in the same line as SEQ ID NO. 58472 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 58668, or a homolog or fragment thereof, or a homolog thereof being depicted in Table I, application no. 17, column 8, in the same line as SEQ ID NO. 58668, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 58668 is depicted, plastidic is mentioned, or a mitochondrial transit peptide in case in Table I, application no. 17, in column 6 in the same line as SEQ ID NO. 58668 is depicted, mitochondric is mentioned; which is operable linked, too.

In another embodiment thereof the expression cassette does not comprise an additional transit peptide in case in Table I, application no. 17, in column 6 in the same line as SEQ ID NO. 58668 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12070, or a homolog or fragment thereof, or a homolog thereof being depicted in Table I, application no. 17, column 8, in the same line as SEQ ID NO. 12070, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 12070 is depicted, plastidic is mentioned, or a mitochondrial transit peptide in case in Table I, application no. 17, in column 6 in the same line as SEQ ID NO. 12070 is depicted, mitochondric is mentioned; which is operable linked, too.

In another embodiment thereof the expression cassette does not comprise an additional transit peptide in case in Table I, application no. 17, in column 6 in the same line as SEQ ID NO. 12070 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 58731, or a homolog or fragment thereof, or a homolog thereof being depicted in Table I, application no. 17, column 8, in the same line as SEQ ID NO. 58731, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 58731 is depicted, plastidic is mentioned, or a mitochondrial transit peptide in case in Table I, application no. 17, in column 6 in the same line as SEQ ID NO. 58731 is depicted, mitochondric is mentioned; which is operable linked, too.

In another embodiment thereof the expression cassette does not comprise an additional transit peptide in case in Table I, application no. 17, in column 6 in the same line as SEQ ID NO. 58731 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 58751, or a homolog or fragment thereof, or a homolog thereof being depicted in Table I, application no. 17, column 8, in the same line as SEQ ID NO. 58751, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 58751 is depicted, plastidic is mentioned, or a mitochondrial transit peptide in case in Table I, application no. 17, in column 6 in the same line as SEQ ID NO. 58751 is depicted, mitochondric is mentioned; which is operable linked, too.

In another embodiment thereof the expression cassette does not comprise an additional transit peptide in case in Table I, application no. 17, in column 6 in the same line as SEQ ID NO. 58751 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 147457, or a homolog or fragment thereof, or a homolog thereof being depicted in Table I, application no. 17, column 8, in the same line as SEQ ID NO. 147457, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 147457 is depicted, plastidic is mentioned, or a mitochondrial transit peptide in case in Table I, application no. 17, in column 6 in the same line as SEQ ID NO. 147457 is depicted, mitochondric is mentioned; which is operable linked, too.

In another embodiment thereof the expression cassette does not comprise an additional transit peptide in case in Table I, application no. 17, in column 6 in the same line as SEQ ID NO. 147457 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 147637, or a homolog or fragment thereof, or a homolog thereof being depicted in Table I, application no. 17, column 8, in the same line as SEQ ID NO. 147637, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 147637 is depicted, plastidic is mentioned, or a mitochondrial transit peptide in case in Table I, application no. 17, in column 6 in the same line as SEQ ID NO. 147637 is depicted, mitochondric is mentioned; which is operable linked, too.

In another embodiment thereof the expression cassette does not comprise an additional transit peptide in case in Table I, application no. 17, in column 6 in the same line as SEQ ID NO. 147637 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12140, or a homolog or fragment thereof, or a homolog thereof being depicted in Table I, application no. 17, column 8, in the same line as SEQ ID NO. 12140, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 12140 is depicted, plastidic is mentioned, or a mitochondrial transit peptide in case in Table I, application no. 17, in column 6 in the same line as SEQ ID NO. 12140 is depicted, mitochondric is mentioned; which is operable linked, too.

In another embodiment thereof the expression cassette does not comprise an additional transit peptide in case in Table I, application no. 17, in column 6 in the same line as SEQ ID NO. 12140 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12341, or a homolog or fragment thereof, or a homolog thereof being depicted in Table I, application no. 17, column 8, in the same line as SEQ ID NO. 12341, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 12341 is depicted, plastidic is mentioned, or a mitochondrial transit peptide in case in Table I, application no. 17, in column 6 in the same line as SEQ ID NO. 12341 is depicted, mitochondric is mentioned; which is operable linked, too.

In another embodiment thereof the expression cassette does not comprise an additional transit peptide in case in Table I, application no. 17, in column 6 in the same line as SEQ ID NO. 12341 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 59370, or a homolog or fragment thereof, or a homolog thereof being depicted in Table I, application no. 17, column 8, in the same line as SEQ ID NO. 59370, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 59370 is depicted, plastidic is mentioned, or a mitochondrial transit peptide in case in Table I, application no. 17, in column 6 in the same line as SEQ ID NO. 59370 is depicted, mitochondric is mentioned; which is operable linked, too.

In another embodiment thereof the expression cassette does not comprise an additional transit peptide in case in Table I, application no. 17, in column 6 in the same line as SEQ ID NO. 59370 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 147836, or a homolog or fragment thereof, or a homolog thereof being depicted in Table I, application no. 17, column 8, in the same line as SEQ ID NO. 147836, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 147836 is depicted, plastidic is mentioned, or a mitochondrial transit peptide in case in Table I, application no. 17, in column 6 in the same line as SEQ ID NO. 147836 is depicted, mitochondric is mentioned; which is operable linked, too.

In another embodiment thereof the expression cassette does not comprise an additional transit peptide in case in Table I, application no. 17, in column 6 in the same line as SEQ ID NO. 147836 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 136907, or a homolog or fragment thereof, or a homolog thereof being depicted in Table I, application no. 17, column 8, in the same line as SEQ ID NO. 136907, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 136907 is depicted, plastidic is mentioned, or a mitochondrial transit peptide in case in Table I, application no. 17, in column 6 in the same line as SEQ ID NO. 136907 is depicted, mitochondric is mentioned; which is operable linked, too.

In another embodiment thereof the expression cassette does not comprise an additional transit peptide in case in Table I, application no. 17, in column 6 in the same line as SEQ ID NO. 136907 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12698, or a homolog or fragment thereof, or a homolog thereof being depicted in Table I, application no. 17, column 8, in the same line as SEQ ID NO. 12698, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 12698 is depicted, plastidic is mentioned, or a mitochondrial transit peptide in case in Table I, application no. 17, in column 6 in the same line as SEQ ID NO. 12698 is depicted, mitochondric is mentioned; which is operable linked, too.

In another embodiment thereof the expression cassette does not comprise an additional transit peptide in case in Table I, application no. 17, in column 6 in the same line as SEQ ID NO. 12698 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 60301, or a homolog or fragment thereof, or a homolog thereof being depicted in Table I, application no. 17, column 8, in the same line as SEQ ID NO. 60301, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 60301 is depicted, plastidic is mentioned, or a mitochondrial transit peptide in case in Table I, application no. 17, in column 6 in the same line as SEQ ID NO. 60301 is depicted, mitochondric is mentioned; which is operable linked, too.

In another embodiment thereof the expression cassette does not comprise an additional transit peptide in case in Table I, application no. 17, in column 6 in the same line as SEQ ID NO. 60301 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 60859, or a homolog or fragment thereof, or a homolog thereof being depicted in Table I, application no. 17, column 8, in the same line as SEQ ID NO. 60859, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 60859 is depicted, plastidic is mentioned, or a mitochondrial transit peptide in case in Table I, application no. 17, in column 6 in the same line as SEQ ID NO. 60859 is depicted, mitochondric is mentioned; which is operable linked, too.

In another embodiment thereof the expression cassette does not comprise an additional transit peptide in case in Table I, application no. 17, in column 6 in the same line as SEQ ID NO. 60859 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 147944, or a homolog or fragment thereof, or a homolog thereof being depicted in Table I, application no. 17, column 8, in the same line as SEQ ID NO. 147944, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 147944 is depicted, plastidic is mentioned, or a mitochondrial transit peptide in case in Table I, application no. 17, in column 6 in the same line as SEQ ID NO. 147944 is depicted, mitochondric is mentioned; which is operable linked, too.

In another embodiment thereof the expression cassette does not comprise an additional transit peptide in case in Table I, application no. 17, in column 6 in the same line as SEQ ID NO. 147944 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 61723, or a homolog or fragment thereof, or a homolog thereof being depicted in Table I, application no. 17, column 8, in the same line as SEQ ID NO. 61723, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 61723 is depicted, plastidic is mentioned, or a mitochondrial transit peptide in case in Table I, application no. 17, in column 6 in the same line as SEQ ID NO. 61723 is depicted, mitochondric is mentioned; which is operable linked, too.

In another embodiment thereof the expression cassette does not comprise an additional transit peptide in case in Table I, application no. 17, in column 6 in the same line as SEQ ID NO. 61723 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 62079, or a homolog or fragment thereof, or a homolog thereof being depicted in Table I, application no. 17, column 8, in the same line as SEQ ID NO. 62079, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 62079 is depicted, plastidic is mentioned, or a mitochondrial transit peptide in case in Table I, application no. 17, in column 6 in the same line as SEQ ID NO. 62079 is depicted, mitochondric is mentioned; which is operable linked, too.

In another embodiment thereof the expression cassette does not comprise an additional transit peptide in case in Table I, application no. 17, in column 6 in the same line as SEQ ID NO. 62079 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12974, or a homolog or fragment thereof, or a homolog thereof being depicted in Table I, application no. 17, column 8, in the same line as SEQ ID NO. 12974, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 12974 is depicted, plastidic is mentioned, or a mitochondrial transit peptide in case in Table I, application no. 17, in column 6 in the same line as SEQ ID NO. 12974 is depicted, mitochondric is mentioned; which is operable linked, too.

In another embodiment thereof the expression cassette does not comprise an additional transit peptide in case in Table I, application no. 17, in column 6 in the same line as SEQ ID NO. 12974 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 148559, or a homolog or fragment thereof, or a homolog thereof being depicted in Table I, application no. 17, column 8, in the same line as SEQ ID NO. 148559, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 148559 is depicted, plastidic is mentioned, or a mitochondrial transit peptide in case in Table I, application no. 17, in column 6 in the same line as SEQ ID NO. 148559 is depicted, mitochondric is mentioned; which is operable linked, too.

In another embodiment thereof the expression cassette does not comprise an additional transit peptide in case in Table I, application no. 17, in column 6 in the same line as SEQ ID NO. 148559 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 62160, or a homolog or fragment thereof, or a homolog thereof being depicted in Table I, application no. 17, column 8, in the same line as SEQ ID NO. 62160, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 62160 is depicted, plastidic is mentioned, or a mitochondrial transit peptide in case in Table I, application no. 17, in column 6 in the same line as SEQ ID NO. 62160 is depicted, mitochondric is mentioned; which is operable linked, too.

In another embodiment thereof the expression cassette does not comprise an additional transit peptide in case in Table I, application no. 17, in column 6 in the same line as SEQ ID NO. 62160 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 62244, or a homolog or fragment thereof, or a homolog thereof being depicted in Table I, application no. 17, column 8, in the same line as SEQ ID NO. 62244, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 62244 is depicted, plastidic is mentioned, or a mitochondrial transit peptide in case in Table I, application no. 17, in column 6 in the same line as SEQ ID NO. 62244 is depicted, mitochondric is mentioned; which is operable linked, too.

In another embodiment thereof the expression cassette does not comprise an additional transit peptide in case in Table I, application no. 17, in column 6 in the same line as SEQ ID NO. 62244 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 62524, or a homolog or fragment thereof, or a homolog thereof being depicted in Table I, application no. 17, column 8, in the same line as SEQ ID NO. 62524, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 62524 is depicted, plastidic is mentioned, or a mitochondrial transit peptide in case in Table I, application no. 17, in column 6 in the same line as SEQ ID NO. 62524 is depicted, mitochondric is mentioned; which is operable linked, too.

In another embodiment thereof the expression cassette does not comprise an additional transit peptide in case in Table I, application no. 17, in column 6 in the same line as SEQ ID NO. 62524 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 88879, or a homolog or fragment thereof, or a homolog thereof being depicted in Table I, application no. 17, column 8, in the same line as SEQ ID NO. 88879, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 88879 is depicted, plastidic is mentioned, or a mitochondrial transit peptide in case in Table I, application no. 17, in column 6 in the same line as SEQ ID NO. 88879 is depicted, mitochondric is mentioned; which is operable linked, too.

In another embodiment thereof the expression cassette does not comprise an additional transit peptide in case in Table I, application no. 17, in column 6 in the same line as SEQ ID NO. 88879 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 89317, or a homolog or fragment thereof, or a homolog thereof being depicted in Table I, application no. 17, column 8, in the same line as SEQ ID NO. 89317, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 89317 is depicted, plastidic is mentioned, or a mitochondrial transit peptide in case in Table I, application no. 17, in column 6 in the same line as SEQ ID NO. 89317 is depicted, mitochondric is mentioned; which is operable linked, too.

In another embodiment thereof the expression cassette does not comprise an additional transit peptide in case in Table I, application no. 17, in column 6 in the same line as SEQ ID NO. 89317 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 96980, or a homolog or fragment thereof, or a homolog thereof being depicted in Table I, application no. 17, column 8, in the same line as SEQ ID NO. 96980, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 96980 is depicted, plastidic is mentioned, or a mitochondrial transit peptide in case in Table I, application no. 17, in column 6 in the same line as SEQ ID NO. 96980 is depicted, mitochondric is mentioned; which is operable linked, too.

In another embodiment thereof the expression cassette does not comprise an additional transit peptide in case in Table I, application no. 17, in column 6 in the same line as SEQ ID NO. 96980 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 89395, or a homolog or fragment thereof, or a homolog thereof being depicted in Table I, application no. 17, column 8, in the same line as SEQ ID NO. 89395, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 89395 is depicted, plastidic is mentioned, or a mitochondrial transit peptide in case in Table I, application no. 17, in column 6 in the same line as SEQ ID NO. 89395 is depicted, mitochondric is mentioned; which is operable linked, too.

In another embodiment thereof the expression cassette does not comprise an additional transit peptide in case in Table I, application no. 17, in column 6 in the same line as SEQ ID NO. 89395 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 108113, or a homolog or fragment thereof, or a homolog thereof being depicted in Table I, application no. 17, column 8, in the same line as SEQ ID NO. 108113, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 108113 is depicted, plastidic is mentioned, or a mitochondrial transit peptide in case in Table I, application no. 17, in column 6 in the same line as SEQ ID NO. 108113 is depicted, mitochondric is mentioned; which is operable linked, too.

In another embodiment thereof the expression cassette does not comprise an additional transit peptide in case in Table I, application no. 17, in column 6 in the same line as SEQ ID NO. 108113 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 148579, or a homolog or fragment thereof, or a homolog thereof being depicted in Table I, application no. 17, column 8, in the same line as SEQ ID NO. 148579, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 148579 is depicted, plastidic is mentioned, or a mitochondrial transit peptide in case in Table I, application no. 17, in column 6 in the same line as SEQ ID NO. 148579 is depicted, mitochondric is mentioned; which is operable linked, too.

In another embodiment thereof the expression cassette does not comprise an additional transit peptide in case in Table I, application no. 17, in column 6 in the same line as SEQ ID NO. 148579 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 63264, or a homolog or fragment thereof, or a homolog thereof being depicted in Table I, application no. 17, column 8, in the same line as SEQ ID NO. 63264, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 63264 is depicted, plastidic is mentioned, or a mitochondrial transit peptide in case in Table I, application no. 17, in column 6 in the same line as SEQ ID NO. 63264 is depicted, mitochondric is mentioned; which is operable linked, too.

In another embodiment thereof the expression cassette does not comprise an additional transit peptide in case in Table I, application no. 17, in column 6 in the same line as SEQ ID NO. 63264 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 122228, or a homolog or fragment thereof, or a homolog thereof being depicted in Table I, application no. 17, column 8, in the same line as SEQ ID NO. 122228, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 122228 is depicted, plastidic is mentioned, or a mitochondrial transit peptide in case in Table I, application no. 17, in column 6 in the same line as SEQ ID NO. 122228 is depicted, mitochondric is mentioned; which is operable linked, too.

In another embodiment thereof the expression cassette does not comprise an additional transit peptide in case in Table I, application no. 17, in column 6 in the same line as SEQ ID NO. 122228 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 120045, or a homolog or fragment thereof, or a homolog thereof being depicted in Table I, application no. 17, column 8, in the same line as SEQ ID NO. 120045, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 120045 is depicted, plastidic is mentioned, or a mitochondrial transit peptide in case in Table I, application no. 17, in column 6 in the same line as SEQ ID NO. 120045 is depicted, mitochondric is mentioned; which is operable linked, too.

In another embodiment thereof the expression cassette does not comprise an additional transit peptide in case in Table I, application no. 17, in column 6 in the same line as SEQ ID NO. 120045 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 14171, or a homolog or fragment thereof, or a homolog thereof being depicted in Table I, application no. 17, column 8, in the same line as SEQ ID NO. 14171, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 14171 is depicted, plastidic is mentioned, or a mitochondrial transit peptide in case in Table I, application no. 17, in column 6 in the same line as SEQ ID NO. 14171 is depicted, mitochondric is mentioned; which is operable linked, too.

In another embodiment thereof the expression cassette does not comprise an additional transit peptide in case in Table I, application no. 17, in column 6 in the same line as SEQ ID NO. 14171 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 137498, or a homolog or fragment thereof, or a homolog thereof being depicted in Table I, application no. 17, column 8, in the same line as SEQ ID NO. 137498, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 137498 is depicted, plastidic is mentioned, or a mitochondrial transit peptide in case in Table I, application no. 17, in column 6 in the same line as SEQ ID NO. 137498 is depicted, mitochondric is mentioned; which is operable linked, too.

In another embodiment thereof the expression cassette does not comprise an additional transit peptide in case in Table I, application no. 17, in column 6 in the same line as SEQ ID NO. 137498 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 14302, or a homolog or fragment thereof, or a homolog thereof being depicted in Table I, application no. 17, column 8, in the same line as SEQ ID NO. 14302, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 14302 is depicted, plastidic is mentioned, or a mitochondrial transit peptide in case in Table I, application no. 17, in column 6 in the same line as SEQ ID NO. 14302 is depicted, mitochondric is mentioned; which is operable linked, too.

In another embodiment thereof the expression cassette does not comprise an additional transit peptide in case in Table I, application no. 17, in column 6 in the same line as SEQ ID NO. 14302 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 89483, or a homolog or fragment thereof, or a homolog thereof being depicted in Table I, application no. 17, column 8, in the same line as SEQ ID NO. 89483, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 89483 is depicted, plastidic is mentioned, or a mitochondrial transit peptide in case in Table I, application no. 17, in column 6 in the same line as SEQ ID NO. 89483 is depicted, mitochondric is mentioned; which is operable linked, too.

In another embodiment thereof the expression cassette does not comprise an additional transit peptide in case in Table I, application no. 17, in column 6 in the same line as SEQ ID NO. 89483 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 148583, or a homolog or fragment thereof, or a homolog thereof being depicted in Table I, application no. 17, column 8, in the same line as SEQ ID NO. 148583, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 148583 is depicted, plastidic is mentioned, or a mitochondrial transit peptide in case in Table I, application no. 17, in column 6 in the same line as SEQ ID NO. 148583 is depicted, mitochondric is mentioned; which is operable linked, too.

In another embodiment thereof the expression cassette does not comprise an additional transit peptide in case in Table I, application no. 17, in column 6 in the same line as SEQ ID NO. 148583 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 113313, or a homolog or fragment thereof, or a homolog thereof being depicted in Table I, application no. 17, column 8, in the same line as SEQ ID NO. 113313, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 113313 is depicted, plastidic is mentioned, or a mitochondrial transit peptide in case in Table I, application no. 17, in column 6 in the same line as SEQ ID NO. 113313 is depicted, mitochondric is mentioned; which is operable linked, too.

In another embodiment thereof the expression cassette does not comprise an additional transit peptide in case in Table I, application no. 17, in column 6 in the same line as SEQ ID NO. 113313 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 63334, or a homolog or fragment thereof, or a homolog thereof being depicted in Table I, application no. 17, column 8, in the same line as SEQ ID NO. 63334, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 63334 is depicted, plastidic is mentioned, or a mitochondrial transit peptide in case in Table I, application no. 17, in column 6 in the same line as SEQ ID NO. 63334 is depicted, mitochondric is mentioned; which is operable linked, too.

In another embodiment thereof the expression cassette does not comprise an additional transit peptide in case in Table I, application no. 17, in column 6 in the same line as SEQ ID NO. 63334 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 63544, or a homolog or fragment thereof, or a homolog thereof being depicted in Table I, application no. 17, column 8, in the same line as SEQ ID NO. 63544, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 63544 is depicted, plastidic is mentioned, or a mitochondrial transit peptide in case in Table I, application no. 17, in column 6 in the same line as SEQ ID NO. 63544 is depicted, mitochondric is mentioned; which is operable linked, too.

In another embodiment thereof the expression cassette does not comprise an additional transit peptide in case in Table I, application no. 17, in column 6 in the same line as SEQ ID NO. 63544 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 90103, or a homolog or fragment thereof, or a homolog thereof being depicted in Table I, application no. 17, column 8, in the same line as SEQ ID NO. 90103, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 90103 is depicted, plastidic is mentioned, or a mitochondrial transit peptide in case in Table I, application no. 17, in column 6 in the same line as SEQ ID NO. 90103 is depicted, mitochondric is mentioned; which is operable linked, too.

In another embodiment thereof the expression cassette does not comprise an additional transit peptide in case in Table I, application no. 17, in column 6 in the same line as SEQ ID NO. 90103 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 90165, or a homolog or fragment thereof, or a homolog thereof being depicted in Table I, application no. 17, column 8, in the same line as SEQ ID NO. 90165, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 90165 is depicted, plastidic is mentioned, or a mitochondrial transit peptide in case in Table I, application no. 17, in column 6 in the same line as SEQ ID NO. 90165 is depicted, mitochondric is mentioned; which is operable linked, too.

In another embodiment thereof the expression cassette does not comprise an additional transit peptide in case in Table I, application no. 17, in column 6 in the same line as SEQ ID NO. 90165 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 148614, or a homolog or fragment thereof, or a homolog thereof being depicted in Table I, application no. 17, column 8, in the same line as SEQ ID NO. 148614, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 148614 is depicted, plastidic is mentioned, or a mitochondrial transit peptide in case in Table I, application no. 17, in column 6 in the same line as SEQ ID NO. 148614 is depicted, mitochondric is mentioned; which is operable linked, too.

In another embodiment thereof the expression cassette does not comprise an additional transit peptide in case in Table I, application no. 17, in column 6 in the same line as SEQ ID NO. 148614 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 97294, or a homolog or fragment thereof, or a homolog thereof being depicted in Table I, application no. 17, column 8, in the same line as SEQ ID NO. 97294, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 97294 is depicted, plastidic is mentioned, or a mitochondrial transit peptide in case in Table I, application no. 17, in column 6 in the same line as SEQ ID NO. 97294 is depicted, mitochondric is mentioned; which is operable linked, too.

In another embodiment thereof the expression cassette does not comprise an additional transit peptide in case in Table I, application no. 17, in column 6 in the same line as SEQ ID NO. 97294 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 128511, or a homolog or fragment thereof, or a homolog thereof being depicted in Table I, application no. 17, column 8, in the same line as SEQ ID NO. 128511, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 128511 is depicted, plastidic is mentioned, or a mitochondrial transit peptide in case in Table I, application no. 17, in column 6 in the same line as SEQ ID NO. 128511 is depicted, mitochondric is mentioned; which is operable linked, too.

In another embodiment thereof the expression cassette does not comprise an additional transit peptide in case in Table I, application no. 17, in column 6 in the same line as SEQ ID NO. 128511 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 148811, or a homolog or fragment thereof, or a homolog thereof being depicted in Table I, application no. 17, column 8, in the same line as SEQ ID NO. 148811, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 148811 is depicted, plastidic is mentioned, or a mitochondrial transit peptide in case in Table I, application no. 17, in column 6 in the same line as SEQ ID NO. 148811 is depicted, mitochondric is mentioned; which is operable linked, too.

In another embodiment thereof the expression cassette does not comprise an additional transit peptide in case in Table I, application no. 17, in column 6 in the same line as SEQ ID NO. 148811 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 148833, or a homolog or fragment thereof, or a homolog thereof being depicted in Table I, application no. 17, column 8, in the same line as SEQ ID NO. 148833, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 148833 is depicted, plastidic is mentioned, or a mitochondrial transit peptide in case in Table I, application no. 17, in column 6 in the same line as SEQ ID NO. 148833 is depicted, mitochondric is mentioned; which is operable linked, too.

In another embodiment thereof the expression cassette does not comprise an additional transit peptide in case in Table I, application no. 17, in column 6 in the same line as SEQ ID NO. 148833 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 64144, or a homolog or fragment thereof, or a homolog thereof being depicted in Table I, application no. 17, column 8, in the same line as SEQ ID NO. 64144, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 64144 is depicted, plastidic is mentioned, or a mitochondrial transit peptide in case in Table I, application no. 17, in column 6 in the same line as SEQ ID NO. 64144 is depicted, mitochondric is mentioned; which is operable linked, too.

In another embodiment thereof the expression cassette does not comprise an additional transit peptide in case in Table I, application no. 17, in column 6 in the same line as SEQ ID NO. 64144 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 149219, or a homolog or fragment thereof, or a homolog thereof being depicted in Table I, application no. 17, column 8, in the same line as SEQ ID NO. 149219, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 149219 is depicted, plastidic is mentioned, or a mitochondrial transit peptide in case in Table I, application no. 17, in column 6 in the same line as SEQ ID NO. 149219 is depicted, mitochondric is mentioned; which is operable linked, too.

In another embodiment thereof the expression cassette does not comprise an additional transit peptide in case in Table I, application no. 17, in column 6 in the same line as SEQ ID NO. 149219 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 120436, or a homolog or fragment thereof, or a homolog thereof being depicted in Table I, application no. 17, column 8, in the same line as SEQ ID NO. 120436, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 120436 is depicted, plastidic is mentioned, or a mitochondrial transit peptide in case in Table I, application no. 17, in column 6 in the same line as SEQ ID NO. 120436 is depicted, mitochondric is mentioned; which is operable linked, too.

In another embodiment thereof the expression cassette does not comprise an additional transit peptide in case in Table I, application no. 17, in column 6 in the same line as SEQ ID NO. 120436 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 90278, or a homolog or fragment thereof, or a homolog thereof being depicted in Table I, application no. 17, column 8, in the same line as SEQ ID NO. 90278, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 90278 is depicted, plastidic is mentioned, or a mitochondrial transit peptide in case in Table I, application no. 17, in column 6 in the same line as SEQ ID NO. 90278 is depicted, mitochondric is mentioned; which is operable linked, too.

In another embodiment thereof the expression cassette does not comprise an additional transit peptide in case in Table I, application no. 17, in column 6 in the same line as SEQ ID NO. 90278 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 64177, or a homolog or fragment thereof, or a homolog thereof being depicted in Table I, application no. 17, column 8, in the same line as SEQ ID NO. 64177, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 64177 is depicted, plastidic is mentioned, or a mitochondrial transit peptide in case in Table I, application no. 17, in column 6 in the same line as SEQ ID NO. 64177 is depicted, mitochondric is mentioned; which is operable linked, too.

In another embodiment thereof the expression cassette does not comprise an additional transit peptide in case in Table I, application no. 17, in column 6 in the same line as SEQ ID NO. 64177 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 130742, or a homolog or fragment thereof, or a homolog thereof being depicted in Table I, application no. 17, column 8, in the same line as SEQ ID NO. 130742, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 130742 is depicted, plastidic is mentioned, or a mitochondrial transit peptide in case in Table I, application no. 17, in column 6 in the same line as SEQ ID NO. 130742 is depicted, mitochondric is mentioned; which is operable linked, too.

In another embodiment thereof the expression cassette does not comprise an additional transit peptide in case in Table I, application no. 17, in column 6 in the same line as SEQ ID NO. 130742 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 14715, or a homolog or fragment thereof, or a homolog thereof being depicted in Table I, application no. 17, column 8, in the same line as SEQ ID NO. 14715, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 14715 is depicted, plastidic is mentioned, or a mitochondrial transit peptide in case in Table I, application no. 17, in column 6 in the same line as SEQ ID NO. 14715 is depicted, mitochondric is mentioned; which is operable linked, too.

In another embodiment thereof the expression cassette does not comprise an additional transit peptide in case in Table I, application no. 17, in column 6 in the same line as SEQ ID NO. 14715 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 64563, or a homolog or fragment thereof, or a homolog thereof being depicted in Table I, application no. 17, column 8, in the same line as SEQ ID NO. 64563, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 64563 is depicted, plastidic is mentioned, or a mitochondrial transit peptide in case in Table I, application no. 17, in column 6 in the same line as SEQ ID NO. 64563 is depicted, mitochondric is mentioned; which is operable linked, too.

In another embodiment thereof the expression cassette does not comprise an additional transit peptide in case in Table I, application no. 17, in column 6 in the same line as SEQ ID NO. 64563 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 149454, or a homolog or fragment thereof, or a homolog thereof being depicted in Table I, application no. 17, column 8, in the same line as SEQ ID NO. 149454, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 149454 is depicted, plastidic is mentioned, or a mitochondrial transit peptide in case in Table I, application no. 17, in column 6 in the same line as SEQ ID NO. 149454 is depicted, mitochondric is mentioned; which is operable linked, too.

In another embodiment thereof the expression cassette does not comprise an additional transit peptide in case in Table I, application no. 17, in column 6 in the same line as SEQ ID NO. 149454 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 64964, or a homolog or fragment thereof, or a homolog thereof being depicted in Table I, application no. 17, column 8, in the same line as SEQ ID NO. 64964, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 64964 is depicted, plastidic is mentioned, or a mitochondrial transit peptide in case in Table I, application no. 17, in column 6 in the same line as SEQ ID NO. 64964 is depicted, mitochondric is mentioned; which is operable linked, too.

In another embodiment thereof the expression cassette does not comprise an additional transit peptide in case in Table I, application no. 17, in column 6 in the same line as SEQ ID NO. 64964 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 97631, or a homolog or fragment thereof, or a homolog thereof being depicted in Table I, application no. 17, column 8, in the same line as SEQ ID NO. 97631, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 97631 is depicted, plastidic is mentioned, or a mitochondrial transit peptide in case in Table I, application no. 17, in column 6 in the same line as SEQ ID NO. 97631 is depicted, mitochondric is mentioned; which is operable linked, too.

In another embodiment thereof the expression cassette does not comprise an additional transit peptide in case in Table I, application no. 17, in column 6 in the same line as SEQ ID NO. 97631 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 149632, or a homolog or fragment thereof, or a homolog thereof being depicted in Table I, application no. 17, column 8, in the same line as SEQ ID NO. 149632, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 149632 is depicted, plastidic is mentioned, or a mitochondrial transit peptide in case in Table I, application no. 17, in column 6 in the same line as SEQ ID NO. 149632 is depicted, mitochondric is mentioned; which is operable linked, too.

In another embodiment thereof the expression cassette does not comprise an additional transit peptide in case in Table I, application no. 17, in column 6 in the same line as SEQ ID NO. 149632 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 97635, or a homolog or fragment thereof, or a homolog thereof being depicted in Table I, application no. 17, column 8, in the same line as SEQ ID NO. 97635, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 97635 is depicted, plastidic is mentioned, or a mitochondrial transit peptide in case in Table I, application no. 17, in column 6 in the same line as SEQ ID NO. 97635 is depicted, mitochondric is mentioned; which is operable linked, too.

In another embodiment thereof the expression cassette does not comprise an additional transit peptide in case in Table I, application no. 17, in column 6 in the same line as SEQ ID NO. 97635 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 149801, or a homolog or fragment thereof, or a homolog thereof being depicted in Table I, application no. 17, column 8, in the same line as SEQ ID NO. 149801, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 149801 is depicted, plastidic is mentioned, or a mitochondrial transit peptide in case in Table I, application no. 17, in column 6 in the same line as SEQ ID NO. 149801 is depicted, mitochondric is mentioned; which is operable linked, too.

In another embodiment thereof the expression cassette does not comprise an additional transit peptide in case in Table I, application no. 17, in column 6 in the same line as SEQ ID NO. 149801 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 14843, or a homolog or fragment thereof, or a homolog thereof being depicted in Table I, application no. 17, column 8, in the same line as SEQ ID NO. 14843, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 14843 is depicted, plastidic is mentioned, or a mitochondrial transit peptide in case in Table I, application no. 17, in column 6 in the same line as SEQ ID NO. 14843 is depicted, mitochondric is mentioned; which is operable linked, too.

In another embodiment thereof the expression cassette does not comprise an additional transit peptide in case in Table I, application no. 17, in column 6 in the same line as SEQ ID NO. 14843 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 149868, or a homolog or fragment thereof, or a homolog thereof being depicted in Table I, application no. 17, column 8, in the same line as SEQ ID NO. 149868, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 149868 is depicted, plastidic is mentioned, or a mitochondrial transit peptide in case in Table I, application no. 17, in column 6 in the same line as SEQ ID NO. 149868 is depicted, mitochondric is mentioned; which is operable linked, too.

In another embodiment thereof the expression cassette does not comprise an additional transit peptide in case in Table I, application no. 17, in column 6 in the same line as SEQ ID NO. 149868 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 150063, or a homolog or fragment thereof, or a homolog thereof being depicted in Table I, application no. 17, column 8, in the same line as SEQ ID NO. 150063, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 150063 is depicted, plastidic is mentioned, or a mitochondrial transit peptide in case in Table I, application no. 17, in column 6 in the same line as SEQ ID NO. 150063 is depicted, mitochondric is mentioned; which is operable linked, too.

In another embodiment thereof the expression cassette does not comprise an additional transit peptide in case in Table I, application no. 17, in column 6 in the same line as SEQ ID NO. 150063 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 130903, or a homolog or fragment thereof, or a homolog thereof being depicted in Table I, application no. 17, column 8, in the same line as SEQ ID NO. 130903, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 130903 is depicted, plastidic is mentioned, or a mitochondrial transit peptide in case in Table I, application no. 17, in column 6 in the same line as SEQ ID NO. 130903 is depicted, mitochondric is mentioned; which is operable linked, too.

In another embodiment thereof the expression cassette does not comprise an additional transit peptide in case in Table I, application no. 17, in column 6 in the same line as SEQ ID NO. 130903 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 98075, or a homolog or fragment thereof, or a homolog thereof being depicted in Table I, application no. 17, column 8, in the same line as SEQ ID NO. 98075, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 98075 is depicted, plastidic is mentioned, or a mitochondrial transit peptide in case in Table I, application no. 17, in column 6 in the same line as SEQ ID NO. 98075 is depicted, mitochondric is mentioned; which is operable linked, too.

In another embodiment thereof the expression cassette does not comprise an additional transit peptide in case in Table I, application no. 17, in column 6 in the same line as SEQ ID NO. 98075 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 98075, or a homolog or fragment thereof, or a homolog thereof being depicted in Table I, application no. 17, column 8, in the same line as SEQ ID NO. 98075, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 98075 is depicted, plastidic is mentioned, or a mitochondrial transit peptide in case in Table I, application no. 17, in column 6 in the same line as SEQ ID NO. 98075 is depicted, mitochondric is mentioned; which is operable linked, too.

In another embodiment thereof the expression cassette does not comprise an additional transit peptide in case in Table I, application no. 17, in column 6 in the same line as SEQ ID NO. 98075 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 150603, or a homolog or fragment thereof, or a homolog thereof being depicted in Table I, application no. 17, column 8, in the same line as SEQ ID NO. 150603, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 150603 is depicted, plastidic is mentioned, or a mitochondrial transit peptide in case in Table I, application no. 17, in column 6 in the same line as SEQ ID NO. 150603 is depicted, mitochondric is mentioned; which is operable linked, too.

In another embodiment thereof the expression cassette does not comprise an additional transit peptide in case in Table I, application no. 17, in column 6 in the same line as SEQ ID NO. 150603 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 91045, or a homolog or fragment thereof, or a homolog thereof being depicted in Table I, application no. 17, column 8, in the same line as SEQ ID NO. 91045, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 91045 is depicted, plastidic is mentioned, or a mitochondrial transit peptide in case in Table I, application no. 17, in column 6 in the same line as SEQ ID NO. 91045 is depicted, mitochondric is mentioned; which is operable linked, too.

In another embodiment thereof the expression cassette does not comprise an additional transit peptide in case in Table I, application no. 17, in column 6 in the same line as SEQ ID NO. 91045 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 150682, or a homolog or fragment thereof, or a homolog thereof being depicted in Table I, application no. 17, column 8, in the same line as SEQ ID NO. 150682, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 150682 is depicted, plastidic is mentioned, or a mitochondrial transit peptide in case in Table I, application no. 17, in column 6 in the same line as SEQ ID NO. 150682 is depicted, mitochondric is mentioned; which is operable linked, too.

In another embodiment thereof the expression cassette does not comprise an additional transit peptide in case in Table I, application no. 17, in column 6 in the same line as SEQ ID NO. 150682 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 91973, or a homolog or fragment thereof, or a homolog thereof being depicted in Table I, application no. 17, column 8, in the same line as SEQ ID NO. 91973, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 91973 is depicted, plastidic is mentioned, or a mitochondrial transit peptide in case in Table I, application no. 17, in column 6 in the same line as SEQ ID NO. 91973 is depicted, mitochondric is mentioned; which is operable linked, too.

In another embodiment thereof the expression cassette does not comprise an additional transit peptide in case in Table I, application no. 17, in column 6 in the same line as SEQ ID NO. 91973 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 130986, or a homolog or fragment thereof, or a homolog thereof being depicted in Table I, application no. 17, column 8, in the same line as SEQ ID NO. 130986, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 130986 is depicted, plastidic is mentioned, or a mitochondrial transit peptide in case in Table I, application no. 17, in column 6 in the same line as SEQ ID NO. 130986 is depicted, mitochondric is mentioned; which is operable linked, too.

In another embodiment thereof the expression cassette does not comprise an additional transit peptide in case in Table I, application no. 17, in column 6 in the same line as SEQ ID NO. 130986 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 117495, or a homolog or fragment thereof, or a homolog thereof being depicted in Table I, application no. 17, column 8, in the same line as SEQ ID NO. 117495, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 117495 is depicted, plastidic is mentioned, or a mitochondrial transit peptide in case in Table I, application no. 17, in column 6 in the same line as SEQ ID NO. 117495 is depicted, mitochondric is mentioned; which is operable linked, too.

In another embodiment thereof the expression cassette does not comprise an additional transit peptide in case in Table I, application no. 17, in column 6 in the same line as SEQ ID NO. 117495 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 67646, or a homolog or fragment thereof, or a homolog thereof being depicted in Table I, application no. 17, column 8, in the same line as SEQ ID NO. 67646, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 67646 is depicted, plastidic is mentioned, or a mitochondrial transit peptide in case in Table I, application no. 17, in column 6 in the same line as SEQ ID NO. 67646 is depicted, mitochondric is mentioned; which is operable linked, too.

In another embodiment thereof the expression cassette does not comprise an additional transit peptide in case in Table I, application no. 17, in column 6 in the same line as SEQ ID NO. 67646 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 15179, or a homolog or fragment thereof, or a homolog thereof being depicted in Table I, application no. 17, column 8, in the same line as SEQ ID NO. 15179, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 15179 is depicted, plastidic is mentioned, or a mitochondrial transit peptide in case in Table I, application no. 17, in column 6 in the same line as SEQ ID NO. 15179 is depicted, mitochondric is mentioned; which is operable linked, too.

In another embodiment thereof the expression cassette does not comprise an additional transit peptide in case in Table I, application no. 17, in column 6 in the same line as SEQ ID NO. 15179 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 151862, or a homolog or fragment thereof, or a homolog thereof being depicted in Table I, application no. 17, column 8, in the same line as SEQ ID NO. 151862, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 151862 is depicted, plastidic is mentioned, or a mitochondrial transit peptide in case in Table I, application no. 17, in column 6 in the same line as SEQ ID NO. 151862 is depicted, mitochondric is mentioned; which is operable linked, too.

In another embodiment thereof the expression cassette does not comprise an additional transit peptide in case in Table I, application no. 17, in column 6 in the same line as SEQ ID NO. 151862 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 92540, or a homolog or fragment thereof, or a homolog thereof being depicted in Table I, application no. 17, column 8, in the same line as SEQ ID NO. 92540, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 92540 is depicted, plastidic is mentioned, or a mitochondrial transit peptide in case in Table I, application no. 17, in column 6 in the same line as SEQ ID NO. 92540 is depicted, mitochondric is mentioned; which is operable linked, too.

In another embodiment thereof the expression cassette does not comprise an additional transit peptide in case in Table I, application no. 17, in column 6 in the same line as SEQ ID NO. 92540 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 151910, or a homolog or fragment thereof, or a homolog thereof being depicted in Table I, application no. 17, column 8, in the same line as SEQ ID NO. 151910, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 151910 is depicted, plastidic is mentioned, or a mitochondrial transit peptide in case in Table I, application no. 17, in column 6 in the same line as SEQ ID NO. 151910 is depicted, mitochondric is mentioned; which is operable linked, too.

In another embodiment thereof the expression cassette does not comprise an additional transit peptide in case in Table I, application no. 17, in column 6 in the same line as SEQ ID NO. 151910 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 151927, or a homolog or fragment thereof, or a homolog thereof being depicted in Table I, application no. 17, column 8, in the same line as SEQ ID NO. 151927, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 151927 is depicted, plastidic is mentioned, or a mitochondrial transit peptide in case in Table I, application no. 17, in column 6 in the same line as SEQ ID NO. 151927 is depicted, mitochondric is mentioned; which is operable linked, too.

In another embodiment thereof the expression cassette does not comprise an additional transit peptide in case in Table I, application no. 17, in column 6 in the same line as SEQ ID NO. 151927 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 151931, or a homolog or fragment thereof, or a homolog thereof being depicted in Table I, application no. 17, column 8, in the same line as SEQ ID NO. 151931, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 151931 is depicted, plastidic is mentioned, or a mitochondrial transit peptide in case in Table I, application no. 17, in column 6 in the same line as SEQ ID NO. 151931 is depicted, mitochondric is mentioned; which is operable linked, too.

In another embodiment thereof the expression cassette does not comprise an additional transit peptide in case in Table I, application no. 17, in column 6 in the same line as SEQ ID NO. 151931 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 151958, or a homolog or fragment thereof, or a homolog thereof being depicted in Table I, application no. 17, column 8, in the same line as SEQ ID NO. 151958, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 151958 is depicted, plastidic is mentioned, or a mitochondrial transit peptide in case in Table I, application no. 17, in column 6 in the same line as SEQ ID NO. 151958 is depicted, mitochondric is mentioned; which is operable linked, too.

In another embodiment thereof the expression cassette does not comprise an additional transit peptide in case in Table I, application no. 17, in column 6 in the same line as SEQ ID NO. 151958 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68413, or a homolog or fragment thereof, or a homolog thereof being depicted in Table I, application no. 17, column 8, in the same line as SEQ ID NO. 68413, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 68413 is depicted, plastidic is mentioned, or a mitochondrial transit peptide in case in Table I, application no. 17, in column 6 in the same line as SEQ ID NO. 68413 is depicted, mitochondric is mentioned; which is operable linked, too.

In another embodiment thereof the expression cassette does not comprise an additional transit peptide in case in Table I, application no. 17, in column 6 in the same line as SEQ ID NO. 68413 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 140626, or a homolog or fragment thereof, or a homolog thereof being depicted in Table I, application no. 17, column 8, in the same line as SEQ ID NO. 140626, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 140626 is depicted, plastidic is mentioned, or a mitochondrial transit peptide in case in Table I, application no. 17, in column 6 in the same line as SEQ ID NO. 140626 is depicted, mitochondric is mentioned; which is operable linked, too.

In another embodiment thereof the expression cassette does not comprise an additional transit peptide in case in Table I, application no. 17, in column 6 in the same line as SEQ ID NO. 140626 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 15187, or a homolog or fragment thereof, or a homolog thereof being depicted in Table I, application no. 17, column 8, in the same line as SEQ ID NO. 15187, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 15187 is depicted, plastidic is mentioned, or a mitochondrial transit peptide in case in Table I, application no. 17, in column 6 in the same line as SEQ ID NO. 15187 is depicted, mitochondric is mentioned; which is operable linked, too.

In another embodiment thereof the expression cassette does not comprise an additional transit peptide in case in Table I, application no. 17, in column 6 in the same line as SEQ ID NO. 15187 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 69, or a homolog or fragment thereof, or a homolog thereof being depicted in Table I, application no. 17, column 8, in the same line as SEQ ID NO. 69, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 69 is depicted, plastidic is mentioned, or a mitochondrial transit peptide in case in Table I, application no. 17, in column 6 in the same line as SEQ ID NO. 69 is depicted, mitochondric is mentioned; which is operable linked, too.

In another embodiment thereof the expression cassette does not comprise an additional transit peptide in case in Table I, application no. 17, in column 6 in the same line as SEQ ID NO. 69 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 16883, or a homolog or fragment thereof, or a homolog thereof being depicted in Table I, application no. 17, column 8, in the same line as SEQ ID NO. 16883, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 16883 is depicted, plastidic is mentioned, or a mitochondrial transit peptide in case in Table I, application no. 17, in column 6 in the same line as SEQ ID NO. 16883 is depicted, mitochondric is mentioned; which is operable linked, too.

In another embodiment thereof the expression cassette does not comprise an additional transit peptide in case in Table I, application no. 17, in column 6 in the same line as SEQ ID NO. 16883 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 149528, or a homolog or fragment thereof, or a homolog thereof being depicted in Table I, application no. 17, column 8, in the same line as SEQ ID NO. 149528, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 149528 is depicted, plastidic is mentioned, or a mitochondrial transit peptide in case in Table I, application no. 17, in column 6 in the same line as SEQ ID NO. 149528 is depicted, mitochondric is mentioned; which is operable linked, too.

In another embodiment thereof the expression cassette does not comprise an additional transit peptide in case in Table I, application no. 17, in column 6 in the same line as SEQ ID NO. 149528 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 150960, or a homolog or fragment thereof, or a homolog thereof being depicted in Table I, application no. 17, column 8, in the same line as SEQ ID NO. 150960, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 17, in column 6 in the same line as SEQ ID NO. 150960 is depicted, plastidic is mentioned, or a mitochondrial transit peptide in case in Table I, application no. 17, in column 6 in the same line as SEQ ID NO. 150960 is depicted, mitochondric is mentioned; which is operable linked, too.

In another embodiment thereof the expression cassette does not comprise an additional transit peptide in case in Table I, application no. 17, in column 6 in the same line as SEQ ID NO. 150960 is depicted, non-targeted is mentioned.

[0165.1.7.17] to [0170.1.7.17] for the disclosure of these paragraphs see [0165.1.7.7] to [0170.1.7.7] above

The invention further provides a vector, comprising a nucleic acid molecule comprising 1) a nucleic acid molecule selected from the group consisting of:
(a) a nucleic acid molecule encoding a polypeptide shown in Table II, column 5 or 8, application no. 17, preferably shown in Table II A, application no. 17, in column 5, or in Table II A, application no. 17, column 8, or in Table II B, application no. 17, column 8, or a homolog or a fragment thereof;
(b) a nucleic acid molecule shown in Table I, application no. 17, column 5 or 8, preferably shown in Table I A, application no. 17, in column 5, or in Table I A, application no. 17, column 8, or in Table I B, application no. 17, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 17, preferably shown in Table II A, application no. 17, in column 5, or in Table II A, application no. 17, column 8, or in Table II B, application no. 17, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 17, preferably shown in Table I A, application no. 17, in column 5, or in Table I A, application no. 17, column 8, or in Table I B, application no. 17, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 17;
(i) a nucleic acid molecule encoding a polypeptide having the activity represented by a protein as depicted in column 5 of Table II, application no. 17;
(j) a nucleic acid molecule comprising a polynucleotide which is obtained by amplifying a cDNA library or a genomic library using the primers in Table III, application no. 17, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 17.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 17, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 17.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 17, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 17, in the same line as of the respective nucleic acid molecule according to 1) “plastidic” is depicted, or encoding a mitochondrial transit peptide, especially in case in column 6 of Table I, application no. 17, in the same line as of the respective nucleic acid molecule according to 1) “mitochondric” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 17, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 17, in the same line as of the respective nucleic acid molecule according to 1) “plastidic” is depicted, or encoding a mitochondrial transit peptide, especially in case in column 6 of Table I, application no. 17, in the same line as of the respective nucleic acid molecule according to 1) “mitochondric” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operable linked; a nucleic acid molecule encoding a transit peptide, especially in case in column 6 of Table I, application no. 17, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 17, in the same line as of the respective nucleic acid molecule according to 1) “plastidic” is depicted, or encoding a mitochondrial transit peptide, especially in case in column 6 of Table I, application no. 17, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 17 (in the same line).

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 17, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 17.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 17, in the same line as of the respective nucleic acid molecule according to 1) “plastidic” is depicted, or encoding a mitochondrial transit peptide, especially in case in column 6 of Table I, application no. 17, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 17, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 17, in the same line as of the respective nucleic acid molecule according to 1) “plastidic” is depicted, or encoding a mitochondrial transit peptide, especially in case in column 6 of Table I, application no. 17, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 17 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 17, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 17 (in the same line) and confers a generation or an increase of the respective fine chemical in a non-human organism or a part thereof as compared to a corresponding wild-type non-human organism or part thereof.

In a preferred embodiment of said vector said nucleic acid molecule according to 1 (a), (b), (c), (d), (e), (f), (g), (h), (i), (j) or (k), said promoter and optionally said nucleic acid molecule encoding a transit peptide are encompassed in an expression cassette. Accordingly in an embodiment the invention provides a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

The invention further provides a vector comprising a nucleic acid molecule comprising 1) a nucleic acid molecule selected from the group consisting of:
(a) a nucleic acid molecule encoding a polypeptide shown in Table II, column 5 or 8, application no. 17, preferably shown in Table II A, application no. 17, in column 5, or in Table II A, application no. 17, column 8, or in Table II B, application no. 17, column 8, or a homolog or a fragment thereof;
(b) a nucleic acid molecule shown in Table I, application no. 17, column 5 or 8, preferably shown in Table I A, application no. 17, in column 5, or in Table I A, application no. 17, column 8, or in Table I B, application no. 17, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 17, preferably shown in Table II A, application no. 17, in column 5, or in Table II A, application no. 17, column 8, or in Table II B, application no. 17, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 17, preferably shown in Table I A, application no. 17, in column 5, or in Table I A, application no. 17, column 8, or in Table I B, application no. 17, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 17;
(i) a nucleic acid molecule encoding a polypeptide having the activity represented by a protein as depicted in column 5 of Table II, application no. 17;
(j) a nucleic acid molecule comprising a polynucleotide which is obtained by amplifying a cDNA library or a genomic library using the primers in Table III, application no. 17, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto;
   whereby, preferably, the nucleic acid molecule according to 1 (a), (b), (c), (d), (e), (f), (g), (h), (i), (j) or (k) distinguishes over the sequence depicted in Table I A and/or I B, application no. 17, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 17, column 5 or 8, or the coding region thereof, respectively. In another embodiment, the nucleic acid molecule of the present invention, preferably the coding region thereof, is 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 17, column 5 or 8, or the coding region thereof, respectively. In a further embodiment the nucleic acid molecule does not encode the polypeptide sequence shown in Table II A and/or II B, application no. 17, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 17, column 5 or 8 and does not encode a protein of the sequence shown in Table II A and/or II B, application no. 17, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 17, column 5 and 8, or the coding region thereof. In a further embodiment, the protein of the present invention is at least 30%,40%, 50%, 60%, 70%, 80%, 90%, 95% identical to protein sequence depicted in Table II A and/or II B, application no. 17, column 5 or 8 but less than 100%, preferably less than 99.999%, 99.99% or 99.9%, more preferably less than 99%, 985, 97%, 96% or 95% identical to the sequence shown in Table II A and/or II B, application no. 17, column 5 or 8.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 17.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 17, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 17.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 17, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 17, in the same line as of the respective nucleic acid molecule according to 1) “plastidic” is depicted, or encoding a mitochondrial transit peptide, especially in case in column 6 of Table I, application no. 17, in the same line as of the respective nucleic acid molecule according to 1) “mitochondric” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 17, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 17, in the same line as of the respective nucleic acid molecule according to 1) “plastidic” is depicted, or encoding a mitochondrial transit peptide, especially in case in column 6 of Table I, application no. 17, in the same line as of the respective nucleic acid molecule according to 1) “mitochondric” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 17, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 17, in the same line as of the respective nucleic acid molecule according to 1) “plastidic” is depicted, or encoding a mitochondrial transit peptide, especially in case in column 6 of Table I, application no. 17, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 17.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 17, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 17.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 17, in the same line as of the respective nucleic acid molecule according to 1) “plastidic” is depicted, or encoding a mitochondrial transit peptide, especially in case in column 6 of Table I, application no. 17, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 17, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 17, in the same line as of the respective nucleic acid molecule according to 1) “plastidic” is depicted, or encoding a mitochondrial transit peptide, especially in case in column 6 of Table I, application no. 17, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 17 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 17, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 17 and confers a generation or an increase of the respective fine chemical in a non-human organism or a part thereof as compared to a corresponding wild-type non-human organism or part thereof.

[0181.1.7.17] to [0209.1.7.17] for the disclosure of these paragraphs see [0181.1.7.7] to [0209.1.7.7] above

The genes of the invention, coding in one embodiment for an activity selected from the group consisting of 1,4-alpha-glucan branching enzyme, 2,3-dihydroxy-2,3-dihydrophenylpropionatedehydrogenase, 2-C-methyl-D-erythritol 4-phosphate cytidylyltransferase, 2-oxoglutarate dehydrogenase E1 subunit, 3-deoxy-7-phosphoheptulonate synthase, 3isopropylmalate dehydratase large subunit, 3-phosphoglycerate dehydrogenase, 47266012-protein, 49747384_SOYBEAN-protein, 4-alpha-glucanotransferase, ABC transporter permease protein, acetyl CoA carboxylase, acetyl CoA synthetase, acetyl-coenzyme A synthetase, acetyltransferase, acid shock protein, acyl carrier protein, acyl transferase, acyl-CoA dehydrogenase, acyl-CoA synthase, adenylosuccinate lyase, alanine dehydrogenase, alcohol dehydrogenase, aldolase, alkaline phosphatase, amine oxidase, amino-acid acetyltransferase, aminotransferase, arginine exporter protein, asparagine synthetase B, At1g19350-protein, At2g45420-protein, At5g16650-protein, At5g43630-protein, ATP synthase subunit E, ATP-binding component of a transport system, auxin response factor, AX653549-protein, AY087308-protein, b0050-protein, b0251-protein, b0362-protein, b0488-protein, B0644-protein, b0801-protein, b0970-protein, b1003-protein, b1046-protein, b1137-protein, b1214-protein, b1219-protein, b1234-protein, b1285-protein, b1357-protein, b1410-protein, b1425-protein, b1445-protein, b1470-protein, b1522-protein, b1556-protein, b1583-protein, b1670-protein, b1796-protein, b1898-protein, b2032-protein, b2107-protein, b2139-protein, b2345-protein, b2360-protein, b2361-protein, b2399-protein, b2466-protein, b2474-protein, b2613-protein, b2653-protein, b2654-protein, b2670-protein, b2673-protein, b2682-protein, b2739-protein, b2932-protein, b3151-protein, b3221-protein, b3246-protein, b3346-protein, B3427-protein, b3442-protein, B3443-protein, b3777-protein, b3817-protein, b3818-protein, b3989-protein, b4029-protein, b4050-protein, b4056-protein, b4250-protein, beta-galactosidase, beta-hydroxylase, betaine aldehyde dehydrogenase, bifunctional protein (phosphoribosyltransferase and regulatory protein), binding-protein-dependent transport systems inner membrane component, branched-chain amino acid ABC transporter permease protein, branched-chain amino acid permease, calcium-dependent protein kinase, carbamoyl-phosphate synthase subunit, cardiolipin synthase, cation-transporting ATPase, CBL-interacting protein kinase, CCAAT-binding transcription factor, CDP-diglyceride synthetase, cell division control protein, cell division protein, chlorophyllase, chorismate lyase, circadian clock protein, colanic acid biosynthesis protein, coproporphyrinogen III oxidase, coproporphyrinogen oxidase, CTP synthase, cullin, cyclin, cyclin D, cystathionine gamma-synthase, cysteine synthase A, dATP pyrophosphohydrolase, dihydrolipoamide dehydrogenase, DNA binding protein, DNA replication complex GINS protein, DnaJ-like chaperone, electron transfer flavoprotein subunit beta, electron transport complex protein, elongation factor Tu, enoyl-CoA hydratase, eukaryotic translation initiation factor, eukaryotic translation initiation factor 5, fatty acid desaturase, ferredoxin, Fe—S subunit of oxidoreductase, flagellar M-ring protein, flavodoxin, fructose biphosphate aldolase, fumarylacetoacetate hydrolase, galactinol synthase, GDP-mannose 4,6-dehydratase, GDP-mannose dehydrogenase, geranylgeranyl pyrophosphate synthase, gibberellin 20-oxidase, Glu/Leu/Phe/Val dehydrogenase, glucokinase, gluconate transport system permease 3, glucose dehydrogenase, glucose-6-phosphate 1-dehydrogenase, glutamate-ammonia-ligase, glutamine amidotransferase, glutamine synthetase, glutaredoxin, glycerate kinase, glycogen (starch) synthase, glycogen synthase, glycosidase, glycosyl transferase, glycosyltransferase, GM02LC38418-protein, GM02LC46-protein, halotolerance protein, heat shock transcription factor, hexokinase, hexose transporter, high-affinity branched-chain amino acid transport protein, histone acetyltransferase, homocitrate synthase, hydrolase, inositol-3-phosphate synthase, integral membrane protein, intracellular septation protein, ion-transport protein, iron(III) dicitrate-binding protein, isochorismate synthase, isocitrate dehydrogenase, isopropylmalate isomerase large subunit, ketol-acid reductoisomerase, kinase, leucyl/phenylalanyl-tRNA-protein transferase, lipoprotein precursor,

L-ribulose-5-phosphate 4-epimerase, L-serine dehydratase, lysine decarboxylase, MADS box protein transcription factor, MADS-box transcription factor, malate dehydrogenase, mannitol-1-phosphate dehydrogenase, membrane protein, membrane transport protein, methylglyoxalase, methyltransferase, Mg-protoporphyrin IX monomethyl ester oxidative cyclase 66 kD subunit, mitogen-activated protein kinase, molecular chaperone, monothiol glutaredoxin, monthiol glutaredoxin, multidrug resistance protein, multiple antibiotic resistance protein, murein transglycosylase, NAD(P)H dehydrogenase (quinone), NADH dehydrogenase, NADH-quinone oxidoreductase subunit, O-acetyltransferase, O-antigen chain length determinant, outer membrane receptor protein precursor, oxidoreductase, oxidoreductase subunit, para-aminobenzoate synthase component I, penicillin-binding protein, peptidyl-prolyl cis-trans isomerase, permease, permease protein of ABC transporter, phage-related repressor protein, phenylacetic acid degradation protein, phenylalanine ammonia-lyase, phosphate acetyltransferase, phosphate permease, phosphatidylinositol 3- and 4-kinase family protein, phosphoadenosine phosphosulfate reductase, phosphoanhydride phosphorylase, phosphoenolpyruvate carboxykinase, phosphoglycerate kinase, phosphohydrolase, phosphotransferase system component, photosystem I reaction center subunit XI, photosystem I reaction centre subunit, photosystem II protein, polyamine transporter, precorrin methylase, protease, protein kinase, protein phosphatase, protein-L-isoaspartate O-methyltransferase, purine biosynthesis protein, purine nucleoside phosphorylase, purine-nucleoside phosphorylase, putative transport system permease protein, putrescine transport system permease protein, pyridoxal phosphate biosynthetic protein, pyrimidine-specific ribonucleoside hydrolase, pyruvate kinase, recombinase A, RNA-binding protein, sec-independent protein translocase, Sec-independent protein translocase subunit, sensor histidine kinase, serine protease, serine/threonine dehydratase, serine/threonine-protein phosphatase, short chain dehydrogenase, short-chain alcohol dehydrogenase family, sll0254-protein, sll0281-protein, sll0354-protein, sll0418-protein, sll1761-protein, slr0600-protein, slr1107-protein, sn-glycerol-3-phosphate dehydrogenase, sn-glycerol-3-phosphate transport system permease protein, sodium/proton antiporter, sterol O-acyltransferase, subunit of COMPASS-protein complex, succinyl-CoA synthetase beta chain, ThiF family protein, thioredoxin, thioredoxin family protein, threonine dehydratase, threonine efflux protein, threonine synthase, transcription factor, transcriptional regulator, transcriptional regulator protein, transport protein, trehalase, trehalose-6-phosphate synthase, trehalose-phosphatase, tryptophan biosynthesis protein, TTC1386-protein, urease accessory protein, urease subunit, vacuolar sorting protein, XM_{—}473199-protein, ycl027w-protein, YCR047W-A-protein, ycr102c-protein, YDL159W-A-protein, ydl235c-protein, ydr183w-protein, ydr507c-protein, YEL030C-A-protein, yer177w-protein, YFL019C-protein, ygl096w-protein, ygl237c-protein, yhl013c-protein, YIL083C-protein, YJL127W-A-protein, ymr324c-protein, yol160w-protein, ypl144w-protein, ypr098c-protein, zinc transporter, and Zm_{—}4842_BE510522-protein are also called “FCRP genes”.

[0211.1.7.17] to [0225.1.7.17] for the disclosure of these paragraphs see [0211.1.7.7] to [0225.1.7.7] above

In addition to the sequence mentioned in Table I, application no. 17, column 5 or 8, preferably the coding region thereof, or its homologs or fragments, it may be advantageous additionally to express and/or mutate further genes in the non-human organisms. Especially advantageously, additionally at least one further gene of the biosynthetic pathway of the fine chemical is expressed in the non-human organisms such as plants or microorganisms. It is also possible that the regulation of the natural genes has been modified advantageously so that the gene and/or its gene product is no longer subject to the regulatory mechanisms which exist in the non-human organisms. This leads to an increased synthesis of the respective saccharide since, for example, feedback regulations no longer exist to the same extent or not at all. In addition it might be advantageously to combine the nucleic acids sequences of the invention containing the sequences shown in the respective line in Table I, application no. 17, column 5 or 8, preferably the coding region thereof, or homologs or fragments thereof, with genes which generally support or enhance the growth or yield of the target non-human organisms, for example genes which lead to faster growth rate of non human organism like microorganisms or plants or genes which produce stress-, pathogen-, or herbicide-resistant plants.

In a further embodiment of the process of the invention, non-human organisms are grown, in which there is in addition to the expression of the nucleic acid molecule according to the invention simultaneous expression of at least one nucleic acid molecule or one of the genes which code for proteins involved in the carbohydrate metabolism, in particular genes of the sugar or starch metabolism, the glucose metabolism, the saccharide metabolism, the metabolism of glycolysis (gluconeogenesis), citrate cycle, pentose-pathway, of glycolipids and glycoprotein metabolism, or their combinations.

In a further embodiment of the process for the production of the fine chemical sorbitol, non-human organisms are grown, in which there is in addition to the expression of the nucleic acid molecule according to the invention simultaneous expression of at least one nucleic acid molecule or one of the genes which code for proteins involved in the sorbitol biosynthesis, e.g. aldose-6-phosphate reductase (EC 1.1.1.200), also called sorbitol-6-phosphate dehydrogenase (S6PDH), D-sorbitol-6-phosphatase, sorbitol-6-phosphate phosphatase (E.C. 3.1.3.50), glucose-fructose oxidoreductase and/or a reduction of the activity of sorbitol dehydrogenase (SDH, EC 1.1.1.14).

In a further embodiment of the process for the production of the fine chemical sucrose, non-human organisms are grown, in which there is in addition to the expression of the nucleic acid molecule according to the invention simultaneous expression of at least one nucleic acid molecule or one of the genes which code for proteins involved in the sucrose biosynthesis, e.g. sucrose-6-phosphate synthase, phosphoglucomutase (EC 5.4.2.2), UDP-glucose pyrophosphorylase (EC 2.7.7.9) and/or sucrose synthase.

In a further embodiment of the process for the production of the fine chemical raffinose, non-human organisms are grown, in which there is in addition to the expression of the nucleic acid molecule according to the invention simultaneous expression of at least one nucleic acid molecule or one of the genes which code for proteins involved in the raffinose biosynthesis, e.g. raffinose synthase, galactinol synthase and/or galactosy transferase.

In a further embodiment of the process for the production of the fine chemical myo-inositol, non-human organisms are grown, in which there is in addition to the expression of the nucleic acid molecule according to the invention simultaneous expression of at least one nucleic acid molecule or one of the genes which code for proteins involved in the myo-inositol biosynthesis, e.g. inositol-1-phosphate synthase and/or inositol monophosphatase.

In a further embodiment of the process for the production of the fine chemical trehalose, non-human organisms are grown, in which there is in addition to the expression of the nucleic acid molecule according to the invention simultaneous expression of at least one nucleic acid molecule or one of the genes which code for proteins involved in the trehalose biosynthesis, e.g. trehalose-6-phosphate synthase (EC 2.4.1.15), trehalose-6-phosphate phophatase and/or trehalose phophatase (EC 3.1.3.12).

In a further embodiment of the process for the production of the fine chemical sedoheptulose-7-phosphate, non-human organisms are grown, in which there is in addition to the expression of the nucleic acid molecule according to the invention simultaneous expression of at least one nucleic acid molecule or one of the genes which code for proteins involved in the sedoheptulose-7-phosphate biosynthesis, e.g. transaldolase [EC 2.2.1.2], Seduheptulose-1,7 bisphosphats phosphatase [EC 3.1.3.37] and/or transketolase [EC 2.2.1.1.

In a further embodiment of the process for the production of the fine chemical mannitol, non-human organisms are grown, in which there is in addition to the expression of the nucleic acid molecule according to the invention simultaneous expression of at least one nucleic acid molecule or one of the genes which code for proteins involved in the mannitol biosynthesis, e.g. mannose-1-phosphate phosphatase [EC 3.1.3.22], mannitol-1-phosphate dehydrogenase, phosphomannose isomerase (E.C. 5.3.1.8) and/or mannose-6-phosphate reductase, M6PR (E.C. 1.1.1.224).

In a further embodiment of the process for the production of the fine chemical glucose, non-human organisms are grown, in which there is in addition to the expression of the nucleic acid molecule according to the invention simultaneous expression of at least one nucleic acid molecule or one of the genes which code for proteins involved in the glucose biosynthesis, e.g. involved in the photosynthesis, involved in the glycogenolysis and/or involved in the gluconeogenesis.

In a further embodiment of the process for the production of the fine chemical galactitol, non-human organisms are grown, in which there is in addition to the expression of the nucleic acid molecule according to the invention simultaneous expression of at least one nucleic acid molecule or one of the genes which code for proteins involved in the galactitol biosynthesis, e.g. enzyme aldose (aldehyde) reductase.

In a further embodiment of the process for the production of the fine chemical galactinol, non-human organisms are grown, in which there is in addition to the expression of the nucleic acid molecule according to the invention simultaneous expression of at least one nucleic acid molecule or one of the genes which code for proteins involved in the galactinol biosynthesis, e.g. proteins involved in the biosynthesis of the RFOs.

In a further embodiment of the process for the production of the fine chemical erythrol, non-human organisms are grown, in which there is in addition to the expression of the nucleic acid molecule according to the invention simultaneous expression of at least one nucleic acid molecule or one of the genes which code for proteins involved in the erythrol biosynthesis, e.g. erythrose reductase genes, being a member of the aldo/keto reductase superfamily and catalysing the NADP(H)-dependent reduction of erythrose to Erythrol.

In a further embodiment of the process for the production of the fine chemical verbascose, non-human organisms are grown, in which there is in addition to the expression of the nucleic acid molecule according to the invention simultaneous expression of at least one nucleic acid molecule or one of the genes which code for proteins involved in the verbascose biosynthesis, e.g. stachyose galactinol:verbascose galactosyltransferase, verbascose galactinol:ajugose galactosyltransferase, stachyose synthase and/or verbascose synthase.

[0228.1.7.17] to [0239.1.7.17] for the disclosure of these paragraphs see [0228.1.7.7] to [0239.1.7.7] above

In an embodiment of the present invention the nucleic acid construct of the present invention comprises a nucleic acid molecule as depicted in the respective line in Table I, application no. 17, column 5 or 8, preferably the coding region thereof, a fragment or a homolog thereof, and a plant promoter, such as the Big 35S promoter, the DC3 promoter, the PC Ubi promoter, the LegB4-promoter, the Super promoter, the USP promoter or the like. In another embodiment the nucleic acid construct of the present invention comprises a nucleic acid molecule as depicted in the respective line in Table I, application no. 17, column 5 or 8, preferably the coding region thereof, a fragment or a homolog thereof, and a plant promoter such as, for example, the USP, the LegB4-, the DC3 promoter or the ubiquitin promoter from parsley or other herein mentioned promoters. In a further embodiment the nucleic acid construct of the present invention comprises a nucleic acid molecule as depicted in the respective line in Table I, application no. 17, column 5 or 8, preferably the coding region thereof, a fragment or a homolog thereof, and a plant promoter, such as the Big 35S promoter, the PC Ubi promoter, the Super promoter, the USP promoter or the like. In addition different terminators may advantageously be used in these nucleic acid constructs; examples for transcriptional termination are polyadenylation signals.

[0241.1.7.17] to [0245.1.7.17] for the disclosure of these paragraphs see [0241.1.7.7] to [0245.1.7.17] as above mentioned

Other preferred sequences for use in operable linkage in gene expression constructs are targeting sequences, which are required for targeting the gene product into specific cell compartments (for a review, see Kermode, Crit. Rev. Plant Sci. 15 (4), 285 (1996) and references cited therein), for example into the vacuole, the nucleus, all types of plastids, such as amyloplasts, chloroplasts, chromoplasts, the extracellular space, the mitochondria, the endoplasmic reticulum, elaioplasts, peroxisomes, glycosomes, and other compartments of cells or extracellular; preferred are sequences, which are involved in targeting to plastids as mentioned above. Sequences, which must be mentioned in this context are, in particular, the signal-peptide- or transit-peptide-encoding sequences which are known per se. For example, plastid-transit-peptide-encoding sequences enable the targeting of the expression product into the plastids of a plant cell. Targeting sequences are also known for eukaryotic and to a lower extent for prokaryotic organisms and can advantageously be operable linked with the nucleic acid molecule of the present invention as shown in Table I, application no. 17, columns 5 and 8, preferably the coding region thereof, or homologs or fragments thereof, and described herein to achieve an expression in one of said compartments or extracellular.

[0247.1.7.17] to [0249.1.7.17] for the disclosure of these paragraphs see [0247.1.7.7] to [0249.1.7.7] above

Preferred promoters are in particular those which bring about gene expression in tissues and organs, such as leaves, roots, seeds, pollen or xylem. Examples of tissue preferred or organ preferred promoters include, but are not limited to fruit-preferred, ovule-preferred, male tissue-preferred, seed-preferred, pollen-preferred integument-preferred, tuber-preferred, stalk-preferred, pericarp-preferred, leaf-preferred, stigma-preferred, pollen-preferred, anther-preferred, petal-preferred, sepal-preferred, pedicel-preferred, silique-preferred, stem-preferred, root-preferred promoters, and the like. Especially those promoters are preferred which bring about gene expression in tissues and organs in which the biosynthesis of saccharides, e.g.carbohydrates takes place, like in seed cells, such as endosperm cells and cells of the developing embryo. Seed promoters are preferentially expressed during seed development and/or germination. For example, seed preferred promoters can be embryo-preferred, endosperm preferred and seed coat-preferred (see Thompson et al., BioEssays 10, 108 (1989)). Examples of seed preferred promoters include, but are not limited to, cellulose synthase (celA), Cim1, gamma-zein, globulin-1, maize 19 kD zein (cZ19B1), and the like. Other suitable promoters are the oilseed rape napin gene promoter (U.S. Pat. No. 5,608,152), the Vicia faba USP promoter (Baeumlein et al., Mol Gen Genet, 225 (3), 459 (1991)), the Arabidopsis oleosin promoter (WO 98/45461), the Phaseolus vulgaris phaseolin promoter (U.S. Pat. No. 5,504,200), the Brassica Bce4 promoter (WO 91/13980), the bean arcs promoter, the carrot DcG3 promoter, or the Legumin B4 promoter (LeB4) (Baeumlein et al., Plant Journal, 2 (2), 233 (1992)), and promoters which bring about the seed-specific expression in monocotyledonous plants such as maize, barley, wheat, rye, rice and the like. Advantageous seed-specific promoters are the sucrose binding protein promoter (WO 00/26388), the phaseolin promoter and the napin promoter. Suitable promoters which must be considered are the barley Ipt2 or Ipt1 gene promoter (WO 95/15389 and WO 95/23230), and the promoters described in WO 99/16890 (promoters from the barley hordein gene, the rice glutelin gene, the rice oryzin gene, the rice prolamin gene, the wheat gliadin gene, the wheat glutelin gene, the maize zein gene, the oat glutelin gene, the sorghum kasirin gene and the rye secalin gene). Further suitable promoters are Amy32b, Amy 6-6 and Aleurain (U.S. Pat. No. 5,677,474), Bce4 (oilseed rape) (U.S. Pat. No. 5,530,149), glycinin (soya) (EP 571 741], phosphoenolpyruvate carboxylase (soya) (JP 06/62870), ADR12-2 (soya) (WO 98/08962), isocitrate lyase (oilseed rape) (U.S. Pat. No. 5,689,040]) or α-amylase (barley) (EP 781 8499. Other promoters which are available for the expression of genes in plants are leaf-specific promoters such as those described in DE-A 19 644 478 or light-regulated promoters such as, for example, the pea petE promoter.

[0251.1.7.17] to [0266.1.7.17] for the disclosure of these paragraphs see [0251.1.7.7] to [0266.1.7.7] above

An embodiment of the invention relates to a vector where the nucleic acid molecule according to the invention is linked operably to regulatory sequences which permit the expression in a prokaryotic or eukaryotic host. A further embodiment of the invention relates to a vector in which a nucleic acid sequence encoding one of the polypeptides shown in the respective line in Table II, application no. 17, columns 5 or 8, or homologs or fragments thereof, is functionally linked to a targeting sequence such as a plastidial or mitochondrial targeting sequence. A further embodiment of the invention relates to a vector in which a nucleic acid sequence encoding one of the polypeptides shown in the respective line in Table II, application no. 17, columns 5 or 8, or homologs or fragments thereof, is functionally linked to (a) regulatory sequence which permit the expression in an organelle, such as plastids or mitochondria, preferably plastids.

A further embodiment of the invention relates to a vector which comprises a nucleic acid sequence encoding one of the polypeptides shown in the respective line in Table II, application no. 17, columns 5 or 8, or homologs or fragments thereof, but no additional targeting sequence.

[0268.1.7.17] to [0273.1.7.17] for the disclosure of these paragraphs see [0268.1.7.7] to [0273.1.7.7] above

A further object of the invention relates to the use of a nucleic acid construct, e.g. an expression cassette, comprising one or more nucleic acid molecules encoding one or more polypeptides as depicted in the respective line(s) in Table II, application no. 17, columns 5 or 8, or homologs or fragments thereof, or comprising one or more nucleic acid molecules as depicted in the respective line(s) in Table I, application no. 17, columns 5 or 8, preferably the coding region thereof, or homologs or fragments thereof, or comprising a nucleic acid molecule hybridizing therewith for the transformation of a microorganism or a plant cell, plant tissues or a part of a plant

In doing so, depending on the choice of promoter, the nucleic acid molecules shown in the respective line in Table I, application no. 17, column 5 or 8, preferably the coding region thereof, or homologs or fragments thereof, or the nucleic acid molecules encoding a polypeptide as depicted in the respective line in Table II, application no. 17, column 5 or 8, or homologs or fragments thereof, can be expressed in the whole plant or specifically e.g. in the leaves, in the seeds, the nodules, in roots, in the stem or other parts of the plant. Those transgenic plants overproducing sequences, e.g. as depicted in the respective line in Table I, application no. 17, column 5 or 8, preferably the coding region thereof, or homologs or fragments thereof, the plant cell, plant tissues, plants, the reproductive material thereof, or parts thereof are a further object of the present invention.

Within the framework of the present invention, the production of the 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose is due to the generation or overexpression of one or more polypeptides as depicted in the respective line(s) in Table II, application no. 17, column 5 or 8, or homologs or fragments thereof, or encoded by the corresponding nucleic acid molecules as depicted in the respective line(s) in Table I, application no. 17, column 5 or 8, preferably the coding region thereof, or homologs or fragments thereof, in the non-human organism according to the invention, advantageously in the transgenic microorganism or plant according to the invention, at least for the duration of at least one plant generation.

A constitutive expression of the polypeptide as depicted in the respective line in Table II, application no. 17, column 5 or 8, or homologs or fragments thereof, or encoded by the respective nucleic acid molecule as depicted in the respective line in Table I, application no. 17, column 5 or 8, preferably the coding region thereof, or homologs or fragments thereof, may be advantageous. On the other hand, an inducible expression may also appear desirable. Expression of the polypeptide sequences of the invention can be either directed to the cytosol or to the organelles, such as plastids or mitochondria, preferably the plastids of the host cells, preferably the plant cells, or non-targeted.

for the disclosure of this paragraph see [0278.1.7.7] above

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 17, column 5 or 8, preferably shown in Table II A, application no. 17, column 5, or in Table II A, application no. 17, column 8, or in Table II B, application no. 17, column 8, or a homolog or a fragment thereof;
   (b) a nucleic acid molecule shown in Table I, application no. 17, column 5 or 8, preferably shown in Table I A, application no. 17, column 5, or in Table I A, application no. 17, column 8, or in Table I B, application no. 17, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 17, preferably shown in Table II A, application no. 17, column 5, or in Table II A, application no. 17, column 8, or in Table II B, application no. 17, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 17, preferably shown in Table I A, application no. 17, column 5, or in Table I A, application no. 17, column 8, or in Table I B, application no. 17, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 17;
   (i) a nucleic acid molecule encoding a polypeptide having the activity represented by a respective protein as depicted in column 5 of Table II, application no. 17;
   (j) a nucleic acid molecule comprising a polynucleotide which is obtained by amplifying a cDNA library or a genomic library using the primers in Table III, application no. 17, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide; which are operable linked;]
   or comprising an expression cassette comprising
[0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 17, column 5 or 8, preferably shown in Table II A, application no. 17, column 5, or in Table II A, application no. 17, column 8, or in Table II B, application no. 17, column 8, or a homolog or a fragment thereof;
   (b) a nucleic acid molecule shown in Table I, application no. 17, column 5 or 8, preferably shown in Table I A, application no. 17, column 5, or in Table I A, application no. 17, column 8, or in Table I B, application no. 17, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 17, preferably shown in Table II A, application no. 17, column 5, or in Table II A, application no. 17, column 8, or in Table II B, application no. 17, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 17, preferably shown in Table I A, application no. 17, column 5, or in Table I A, application no. 17, column 8, or in Table I B, application no. 17, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 17;
   (i) a nucleic acid molecule encoding a polypeptide having the activity represented by a respective protein as depicted in column 5 of Table II, application no. 17;
   (j) a nucleic acid molecule comprising a polynucleotide which is obtained by amplifying a cDNA library or a genomic library using the primers in Table III, application no. 17, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide; which are operable linked;]
   or comprising or being transformed by a vector comprising
[1) a nucleic acid molecule selected from the group consisting of
   (a) a nucleic acid molecule encoding a polypeptide shown in Table II, application no. 17, column 5 or 8, preferably shown in Table II A, application no. 17, column 5, or in Table II A, application no. 17, column 8, or in Table II B, application no. 17, column 8, or a homolog or a fragment thereof;
   (b) a nucleic acid molecule shown in Table I, application no. 17, column 5 or 8, preferably shown in Table I A, application no. 17, column 5, or in Table I A, application no. 17, column 8, or in Table I B, application no. 17, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 17, preferably shown in Table II A, application no. 17, column 5, or in Table II A, application no. 17, column 8, or in Table II B, application no. 17, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 17, preferably shown in Table I A, application no. 17, column 5, or in Table I A, application no. 17, column 8, or in Table I B, application no. 17, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 17;
   (i) a nucleic acid molecule encoding a polypeptide having the activity represented by a respective protein as depicted in column 5 of Table II, application no. 17;
   (j) a nucleic acid molecule comprising a polynucleotide which is obtained by amplifying a cDNA library or a genomic library using the primers in Table III, application no. 17, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 17.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 17 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 17, in the same line as of the respective nucleic acid molecule according to 1) “plastidic” is depicted, or encoding a mitochondrial transit peptide, especially in case in column 6 of Table I, application no. 17, in the same line as of the respective nucleic acid molecule according to 1) “mitochondric” is depicted.

In another embodiment of said transgenic non-human organism the expression cassette does not comprise 2) a nucleic acid molecule encoding a transit peptide, especially in case in column 6 of Table I, application no. 17, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 17.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 17, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 17, in the same line as of the respective nucleic acid molecule according to 1) “plastidic” is depicted, or encoding a mitochondrial transit peptide, especially in case in column 6 of Table I, application no. 17, in the same line as of the respective nucleic acid molecule according to 1) “mitochondric” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 17, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 17, in the same line as of the respective nucleic acid molecule according to 1) “plastidic” is depicted, or encoding a mitochondrial transit peptide, especially in case in column 6 of Table I, application no. 17, in the same line as of the respective nucleic acid molecule according to 1) “mitochondric” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 17, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 17, in the same line as of the respective nucleic acid molecule according to 1) “plastidic” is depicted, or encoding a mitochondrial transit peptide, especially in case in column 6 of Table I, application no. 17, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 17.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 17, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 17.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 17, in the same line as of the respective nucleic acid molecule according to 1) “plastidic” is depicted, or encoding a mitochondrial transit peptide, especially in case in column 6 of Table I, application no. 17, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 17, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 17, in the same line as of the respective nucleic acid molecule according to 1) “plastidic” is depicted, or encoding a mitochondrial transit peptide, especially in case in column 6 of Table I, application no. 17, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 17 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 17, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 17 (in the same line) and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 17, column 5 or 8, preferably shown in Table II A, application no. 17, column 5, or in Table II A, application no. 17, column 8, or in Table II B, application no. 17, column 8, or a homolog or a fragment thereof;
   (b) a nucleic acid molecule shown in Table I, application no. 17, column 5 or 8, preferably shown in Table I A, application no. 17, column 5, or in Table I A, application no. 17, column 8, or in Table I B, application no. 17, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 17, preferably shown in Table II A, application no. 17, column 5, or in Table II A, application no. 17, column 8, or in Table II B, application no. 17, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 17, preferably shown in Table I A, application no. 17, column 5, or in Table I A, application no. 17, column 8, or in Table I B, application no. 17, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 17;
   (i) a nucleic acid molecule encoding a polypeptide having the activity represented by a respective protein as depicted in column 5 of Table II, application no. 17;
   (j) a nucleic acid molecule comprising a polynucleotide which is obtained by amplifying a cDNA library or a genomic library using the primers in Table III, application no. 17, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising an expression cassette comprising
[0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 17, column 5 or 8, preferably shown in Table II A, application no. 17, column 5, or in Table II A, application no. 17, column 8, or in Table II B, application no. 17, column 8, or a homolog or a fragment thereof;
   (b) a nucleic acid molecule shown in Table I, application no. 17, column 5 or 8, preferably shown in Table I A, application no. 17, column 5, or in Table I A, application no. 17, column 8, or in Table I B, application no. 17, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 17, preferably shown in Table II A, application no. 17, column 5, or in Table II A, application no. 17, column 8, or in Table II B, application no. 17, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 17, preferably shown in Table I A, application no. 17, column 5, or in Table I A, application no. 17, column 8, or in Table I B, application no. 17, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 17;
   (i) a nucleic acid molecule encoding a polypeptide having the activity represented by a respective protein as depicted in column 5 of Table II, application no. 17;
   (j) a nucleic acid molecule comprising a polynucleotide which is obtained by amplifying a cDNA library or a genomic library using the primers in Table III, application no. 17, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      and, if desired
2) nucleic acid molecule encoding a transit peptide, preferably encoding a plastidal transit peptide or a mitochondrial transit peptide; which are operable linked;]
   or comprising or being transformed by a vector comprising
   [1) a nucleic acid molecule selected from the group consisting of
   (a) a nucleic acid molecule encoding a polypeptide shown in Table II, application no. 17, column 5 or 8, preferably shown in Table II A, application no. 17, column 5, or in Table II A, application no. 17, column 8, or in Table II B, application no. 17, column 8, or a homolog or a fragment thereof;
   (b) a nucleic acid molecule shown in Table I, application no. 17, column 5 or 8, preferably shown in Table I A, application no. 17, column 5, or in Table I A, application no. 17, column 8, or in Table I B, application no. 17, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 17, preferably shown in Table II A, application no. 17, column 5, or in Table II A, application no. 17, column 8, or in Table II B, application no. 17, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 17, preferably shown in Table I A, application no. 17, column 5, or in Table I A, application no. 17, column 8, or in Table I B, application no. 17, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 17;
   (i) a nucleic acid molecule encoding a polypeptide having the activity represented by a respective protein as depicted in column 5 of Table II, application no. 17;
   (j) a nucleic acid molecule comprising a polynucleotide which is obtained by amplifying a cDNA library or a genomic library using the primers in Table III, application no. 17, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      whereby, preferably, the nucleic acid molecule according to 1 (a), (b), (c), (d), (e), (f), (g), (h), (i), (j) or (k) distinguishes over the sequence depicted in Table I A and/or I B, application no. 17, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 17, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 17, column 5 or 8, or the coding region thereof, respectively. In a further embodiment the nucleic acid molecule does not encode the polypeptide sequence shown in Table II A and/or II B, application no. 17, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 17, column 5 or 8 and does not encode a protein of the sequence shown in Table II A and/or II B, application no. 17, column 5 or 8. Accordingly, in one embodiment, the protein encoded by a sequence of a nucleic acid according to 1 (a), (b), (c), (d), (e), (f), (g), (h), (i), (j) or (k) does not consist of the sequence shown in Table I A and/or I B, application no. 17, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 17, column 5 or 8 but less than 100%, preferably less than 99.999%, 99.99%, 99.9%, 99%, 98%, 97%, 96% or 95% identical to the sequence shown in Table II A and/or II B, application no. 17, column 5 or 8; as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 17.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 17 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 17, in the same line as of the respective nucleic acid molecule according to 1) “plastidic” is depicted, or encoding a mitochondrial transit peptide, especially in case in column 6 of Table I, application no. 17, in the same line as of the respective nucleic acid molecule according to 1) “mitochondric” is depicted.

In another embodiment thereof the expression cassette does not comprise 2) a nucleic acid molecule encoding a transit peptide, especially in case in column 6 of Table I, application no. 17, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 17.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the same line in column 5 of Table II, application no. 17, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 17, in the same line as of the respective nucleic acid molecule according to 1) “plastidic” is depicted, or encoding a mitochondrial transit peptide, especially in case in column 6 of Table I, application no. 17, in the same line as of the respective nucleic acid molecule according to 1) “mitochondric” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 17, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 17, in the same line as of the respective nucleic acid molecule according to 1) “plastidic” is depicted, or encoding a mitochondrial transit peptide, especially in case in column 6 of Table I, application no. 17, in the same line as of the respective nucleic acid molecule according to 1) “mitochondric” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 17, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 17, in the same line as of the respective nucleic acid molecule according to 1) “plastidic” is depicted, or encoding a mitochondrial transit peptide, especially in case in column 6 of Table I, application no. 17, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 17.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 17, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 17.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 17, in the same line as of the respective nucleic acid molecule according to 1) “plastidic” is depicted, or encoding a mitochondrial transit peptide, especially in case in column 6 of Table I, application no. 17, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 17, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 17, in the same line as of the respective nucleic acid molecule according to 1) “plastidic” is depicted, or encoding a mitochondrial transit peptide, especially in case in column 6 of Table I, application no. 17, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 17 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 17, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 17 and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

[0291.1.7.17] to [0299.1.7.17] for the disclosure of these paragraphs see [0291.1.7.7] to [0299.1.7.7] above

The term “biological active portion” or “biological activity” means a polypeptide as depicted in the respective line in Table II, application no. 17, column 3 or a portion of said polypeptide which still has at least 10% or 20%, preferably 30%, 40%, 50% or 60%, especially preferably 70%, 75%, 80%, 90% or 95% of the enzymatic or biological activity of the natural or starting enzyme or protein.

[0301.1.7.17] to [0304.1.7.17] for the disclosure of these paragraphs see [0301.1.7.7] to [0304.1.7.7] above

Synthetic oligonucleotide primers for the amplification, e.g. as shown in the respective line in Table III, application no. 17, column 8, by means of polymerase chain reaction can be generated on the basis of a sequence shown herein, for example the sequence shown in the respective line in Table I, application no. 17, columns 5 or 8, or the sequences derived from Table II, application no. 17, columns 5 or 8, respectively.

Moreover, it is possible to identify a conserved region of a protein from various organism by carrying out protein sequence alignments with the polypeptide encoded by the nucleic acid molecules of the present invention, in particular with the sequences encoded by the nucleic acid molecule shown in the respective line in column 5 or 8 of Table I, application no. 17, from which conserved regions, and in turn, degenerate primers can be derived. Conserved regions are those, which show a very little variation in the amino acid in one particular position of several homologs from different origin. The consensus sequence and polypeptide motifs shown in the respective line in column 8 of Table IV, application no. 17, are derived from such alignments of homologous proteins.

In an embodiment of the present invention, in the method of the present invention the activity of a polypeptide comprising or consisting of a consensus sequence or at least one polypeptide motif shown in the respective line in Table IV, application no. 17, column 8, is increased. In another embodiment thereof, the method of the present invention relates to a polypeptide comprising or consisting of a consensus sequence shown in the respective line in Table IV, application no. 17, column 8 or comprising at least one polypeptide motif shown in the respective line in Table IV, application no. 17, column 8, whereby not more than 20, 15, 10, 9, 8, 7, 6, 5 4, 3, 2 or 1, or 0 of the amino acid positions indicated can be replaced by any amino acid. In another embodiment thereof, the method of the present invention relates to a polypeptide comprising or consisting of a consensus sequence shown in the respective line in Table IV, application no. 17, column 8 or comprising at least one polypeptide motif shown in the respective line in Table IV, application no. 17, column 8, whereby not more than 15%, 10%, 5%, 4%, 3%, 2% or 1%, or 0% of the amino acid positions defined as distinct specific amino acids are/is replaced by another amino acid. In another embodiment thereof, the method of the present invention relates to a polypeptide comprising or consisting of a consensus sequence shown in the respective line in Table IV, application no. 17, column 8 or comprising at least one polypeptide motif shown in the respective line in Table IV, application no. 17, column 8, whereby not more than 20, 15, 10, 9, 8, 7, 6, 5, 4, 3, 2 or 1, or 0 amino acids are inserted into a consensus sequence or polypeptide motif.

In an embodiment of the present invention, in the method of the present invention the activity of a polypeptide comprising or consisting of a consensus sequence or at least one polypeptide motif shown in the respective line in Table IV, application no. 17, column 8, is increased, whereby said polypeptide distinguishes over the sequence depicted in Table II, application no. 17, columns 5 or 8 by one or more amino acids.

In one embodiment, said polypeptide of the invention distinguishes over the sequence shown in the respective line in Table II, application no. 17, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II B, application no. 17, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in Table II, application no. 17, columns 5 or 8.

[0309.1.7.17] to [0321.1.7.17] for the disclosure of this paragraph see [0309.1.7.7] to [0321.1.7.7] above

Polypeptides having above-mentioned activity, i.e. conferring the production or the increased production of the fine chemical 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose as compared to a corresponding, e.g. non-transformed, wild type non-human organism, like a microorganism or a plant cell, plant or part thereof, can be encoded by other DNA sequences which hybridize to the sequences shown in the respective line in Table I, application no. 17, columns 5 and 8, preferably the coding region thereof, at least under relaxed hybridization conditions and which encode the expression of polypeptides conferring the production or the increased production of the respective fine chemical 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose as compared to a corresponding, e.g. non-transformed, wild type non-human organism, like a microorganism or a plant cell, plant or part thereof.

[0323.1.7.17] to [0329.1.7.17] for the disclosure of these paragraphs see [0323.1.7.7] to [0329.1.7.7] above

Further, the nucleic acid molecule of the invention comprises a nucleic acid molecule, which is a complement of one of the nucleotide sequences of above-mentioned nucleic acid molecules or a portion thereof. A nucleic acid molecule or its sequence which is complementary to one of the nucleotide molecules or sequences shown in the respective line in Table I, application no. 17, columns 5 or 8, preferably the coding region thereof, homologs or fragments thereof, is one which is sufficiently complementary to one of the nucleotide molecules or sequences shown in the respective line in Table I, application no. 17, columns 5 or 8, preferably the coding region thereof, homologs or fragments thereof, such that it can hybridize to one of the nucleotide sequences shown in Table I, application no. 17, columns 5 or 8, preferably the coding region thereof, homologs or fragments thereof, thereby forming a stable duplex. Preferably, the hybridization is performed under stringent hybridization conditions. However, a complement of one of the herein disclosed sequences is preferably a sequence complement thereto according to the base pairing of nucleic acid molecules well known to the skilled person.

For example, the bases A and G undergo base pairing with the bases T and U or C, resp. and visa versa. Modifications of the bases can influence the base-pairing partner.

The nucleic acid molecule of the invention comprises a nucleotide sequence which is at least about 30%, 35%, 40%, 45%, 50%, 55%, 60%, 65%, 70%, 75%, 80%, 90%, 95%, 96%, 97%, 98%, 99%, 99.5% or more homologous to a nucleotide sequence shown in Table I, application no. 17, columns 5 or 8, preferably the coding region thereof, homologs or fragments thereof, and preferably has above-mentioned activity, in particular conferring the production or the increased production of the fine chemical 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose, respectively, after increasing the activity or an activity of a gene as shown in the respective line in Table I or of a gene product, e.g. as shown in the respective line in Table II, application no. 17, column 5 or 8, by for example in one embodiment expression either in the cytosol or in an organelle such as a plastid or mitochondria or both, preferably in a plastid, or in another embodiment non-targeted or targeted.

The nucleic acid molecule of the invention comprises a nucleotide sequence or molecule which hybridizes, preferably hybridizes under stringent conditions as defined herein, to one of the nucleic acid molecule shown in the respective line in Table I, preferably Table IB, application no. 17, columns 5 or 8, preferably the coding region thereof, fragments or homologs thereof, and encodes a protein having above-mentioned activity, e.g. conferring the production or the increased production of the respective fine chemical 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted, and optionally, the activity thereof is selected from the group consisting of 1,4-alpha-glucan branching enzyme, 2,3-dihydroxy-2,3-dihydrophenylpropionatedehydrogenase, 2-C-methyl-D-erythritol 4-phosphate cytidylyltransferase, 2-oxoglutarate dehydrogenase E1 subunit, 3-deoxy7-phosphoheptulonate synthase, 3-isopropylmalate dehydratase large subunit, 3-phosphoglycerate dehydrogenase, 47266012-protein, 49747384_SOYBEAN-protein, 4-alpha-glucanotransferase, ABC transporter permease protein, acetyl CoA carboxylase, acetyl CoA synthetase, acetyl-coenzyme A synthetase, acetyltransferase, acid shock protein, acyl carrier protein, acyl transferase, acyl-CoA dehydrogenase, acyl-CoA synthase, adenylosuccinate lyase, alanine dehydrogenase, alcohol dehydrogenase, aldolase, alkaline phosphatase, amine oxidase, amino-acid acetyltransferase, aminotransferase, arginine exporter protein, asparagine synthetase B, At1g19350-protein, At2g45420-protein, At5g16650-protein, At5g43630-protein, ATP synthase subunit E, ATP-binding component of a transport system, auxin response factor, AX653549-protein, AY087308-protein, b0050-protein, b0251-protein, b0362-protein, b0488-protein, B0644-protein, b0801-protein, b0970-protein, b1003-protein, b1046-protein, b1137-protein, b1214-protein, b1219-protein, b1234-protein, b1285-protein, b1357-protein, b1410-protein, b1425-protein, b1445-protein, b1470-protein, b1522-protein, b1556-protein, b1583-protein, b1670-protein, b1796-protein, b1898-protein, b2032-protein, b2107-protein, b2139-protein, b2345-protein, b2360-protein, b2361-protein, b2399-protein, b2466-protein, b2474-protein, b2613-protein, b2653-protein, b2654-protein, b2670-protein, b2673-protein, b2682-protein, b2739-protein, b2932-protein, b3151-protein, b3221-protein, b3246-protein, b3346-protein, B3427-protein, b3442-protein, B3443-protein, b3777-protein, b3817-protein, b3818-protein, b3989-protein, b4029-protein, b4050-protein, b4056-protein, b4250-protein, beta-galactosidase, beta-hydroxylase, betaine aldehyde dehydrogenase, bifunctional protein (phosphoribosyltransferase and regulatory protein), binding-protein-dependent transport systems inner membrane component, branched-chain amino acid ABC transporter permease protein, branched-chain amino acid permease, calcium-dependent protein kinase, carbamoyl-phosphate synthase subunit, cardiolipin synthase, cation-transporting ATPase, CBL-interacting protein kinase, CCAAT-binding transcription factor, CDP-diglyceride synthetase, cell division control protein, cell division protein, chlorophyllase, chorismate lyase, circadian clock protein, colanic acid biosynthesis protein, coproporphyrinogen III oxidase, coproporphyrinogen oxidase, CTP synthase, cullin, cyclin, cyclin D, cystathionine gamma-synthase, cysteine synthase A, dATP pyrophosphohydrolase, dihydrolipoamide dehydrogenase, DNA binding protein, DNA replication complex GINS protein, DnaJ-like chaperone, electron transfer flavoprotein subunit beta, electron transport complex protein, elongation factor Tu, enoyl-CoA hydratase, eukaryotic translation initiation factor, eukaryotic translation initiation factor 5, fatty acid desaturase, ferredoxin, Fe—S subunit of oxidoreductase, flagellar M-ring protein, flavodoxin, fructose biphosphate aldolase, fumarylacetoacetate hydrolase, galactinol synthase, GDP-mannose 4,6-dehydratase, GDP-mannose dehydrogenase, geranylgeranyl pyrophosphate synthase, gibberellin 20-oxidase, Glu/Leu/Phe/Val dehydrogenase , glucokinase, gluconate transport system permease 3, glucose dehydrogenase, glucose-6-phosphate 1-dehydrogenase, glutamate-ammonia-ligase, glutamine amidotransferase, glutamine synthetase, glutaredoxin, glycerate kinase, glycogen (starch) synthase, glycogen synthase, glycosidase, glycosyl transferase, glycosyltransferase, GM02LC38418-protein, GM02LC46-protein, halotolerance protein, heat shock transcription factor, hexokinase, hexose transporter, high-affinity branched-chain amino acid transport protein, histone acetyltransferase, homocitrate synthase, hydrolase, inositol-3-phosphate synthase, integral membrane protein, intracellular septation protein, ion-transport protein, iron(III) dicitrate-binding protein, isochorismate synthase, isocitrate dehydrogenase, isopropylmalate isomerase large subunit, ketol-acid reductoisomerase, kinase, leucyl/phenylalanyl-tRNA-protein transferase, lipoprotein precursor, L-ribulose-5-phosphate 4-epimerase, L-serine dehydratase, lysine decarboxylase, MADS box protein transcription factor, MADS-box transcription factor, malate dehydrogenase, mannitol-1-phosphate dehydrogenase, membrane protein, membrane transport protein, methylglyoxalase, methyltransferase, Mg-protoporphyrin IX monomethyl ester oxidative cyclase 66 kD subunit, mitogen-activated protein kinase, molecular chaperone, monothiol glutaredoxin, monthiol glutaredoxin, multidrug resistance protein, multiple antibiotic resistance protein, murein transglycosylase, NAD(P)H dehydrogenase (quinone), NADH dehydrogenase, NADH-quinone oxidoreductase subunit, O-acetyltransferase, O-antigen chain length determinant, outer membrane receptor protein precursor, oxidoreductase, oxidoreductase subunit, para-aminobenzoate synthase component I, penicillin-binding protein, peptidyl-prolyl cis-trans isomerase, permease, permease protein of ABC transporter, phage-related repressor protein, phenylacetic acid degradation protein, phenylalanine ammonia-lyase, phosphate acetyltransferase, phosphate permease, phosphatidylinositol 3- and 4-kinase family protein, phosphoadenosine phosphosulfate reductase, phosphoanhydride phosphorylase, phosphoenolpyruvate carboxykinase, phosphoglycerate kinase, phosphohydrolase, phosphotransferase system component, photosystem I reaction center subunit XI, photosystem I reaction centre subunit, photosystem IIprotein, polyamine transporter, precorrin methylase, protease, protein kinase, protein phosphatase, protein-L-isoaspartate O-methyltransferase, purine biosynthesis protein, purine nucleoside phosphorylase, purine-nucleoside phosphorylase, putative transport system permease protein, putrescine transport system permease protein, pyridoxal phosphate biosynthetic protein, pyrimidine-specific ribonucleoside hydrolase, pyruvate kinase, recombinase A, RNA-binding protein, sec-independent protein translocase, Sec-independent protein translocase subunit, sensor histidine kinase, serine protease, serine/threonine dehydratase, serine/threonine-protein phosphatase, short chain dehydrogenase, short-chain alcohol dehydrogenase family, sll0254-protein, sll0281-protein, sll0354-protein, sll0418-protein, sll1761-protein, slr0600-protein, slr1107-protein, sn-glycerol-3-phosphate dehydrogenase, sn-glycerol-3-phosphate transport system permease protein, sodium/proton antiporter, sterol O-acyltransferase, subunit of COMPASS-protein complex, succinyl-CoA synthetase beta chain, ThiF family protein, thioredoxin, thioredoxin family protein, threonine dehydratase, threonine efflux protein, threonine synthase, transcription factor, transcriptional regulator, transcriptional regulator protein, transport protein, trehalase, trehalose-6-phosphate synthase, trehalosephosphatase, tryptophan biosynthesis protein, TTC1386-protein, urease accessory protein, urease subunit, vacuolar sorting protein, XM_{—}473199-protein, ycl027w-protein, YCR047W-A-protein, ycr102c-protein, YDL159W-A-protein, ydl235c-protein, ydr183w-protein, ydr507c-protein, YEL030C-A-protein, yer177w-protein, YFL019C-protein, ygl096w-protein, ygl237c-protein, yhl013c-protein, YIL083C-protein, YJL127W-A-protein, ymr324c-protein, yol160w-protein, ypl144w-protein, ypr098c-protein, zinc transporter, and Zm_{—}4842_BE510522-protein, respectively.

Moreover, the nucleic acid molecule of the invention can comprise only a portion of the coding region of one of the nucleic acid molecules depicted in Table I, application no. 17, columns 5 or 8, for example a fragment which can be used as a probe or primer or a fragment encoding a biologically active portion of the polypeptide of the present invention or of a polypeptide used in the process of the present invention, i.e. having above-mentioned activity, e.g. conferring the production or the increased production of the respective fine chemical 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted. The nucleotide sequences determined from the cloning of the present protein-according-to-the-invention-encoding gene allows for the generation of probes and primers designed for use in identifying and/or cloning its homologues from other cell types and organisms. The probe/primer typically comprises a substantially purified oligonucleotide. The oligonucleotide typically comprises a region of nucleotide sequence that hybridizes under stringent conditions to at least about 12, 15 preferably about 20 or 25, more preferably about 40, 50 or 75 consecutive nucleotides of a sense strand of one of the sequences set forth, e.g., in the respective line in Table I, application no. 17, column 5 or 8, preferably the coding region thereof, or a homolog or a fragment thereof, an anti-sense sequence of one of the sequences, e.g., set forth in the respective line in Table I, application no. 17, columns 5 or 8, preferably the coding region thereof, or a homolog or a fragment thereof or naturally occurring mutants thereof. Primers based on a nucleic acid molecule of invention can be used in PCR reactions to clone homologs of the polypeptide of the invention or of the polypeptide used in the process of the invention, e.g. as the primers described in the examples of the present invention, e.g. as shown in the examples. A PCR with the primers shown in the respective line in Table Ill, column 8 will result in a fragment of the gene product as shown in Table II, application no. 17, column 5.

Primer sets are interchangeable. The person skilled in the art knows how to design and combine said primers to result in the desired product, e.g. in a full length clone or a partial sequence. Probes based on the sequences of the nucleic acid molecule of the invention or used in the process of the present invention can be used to detect transcripts or genomic sequences encoding the same or homologous proteins. The probe can further comprise a label group attached thereto, e.g. the label group can be a radioisotope, a fluorescent compound, an enzyme, or an enzyme co-factor. Such probes can be used as a part of a genomic marker test kit for identifying cells which express a polypeptide of the invention or used in the process of the present invention, such as by measuring a level of an encoding nucleic acid molecule in a sample of cells, e.g. detecting mRNA levels or determining, whether a genomic gene comprising the sequence of the polynucleotide of the invention or used in the processes of the present invention has been mutated or deleted.

As the expression of a respective nucleic acids of the invention is related to the synthesis of the respective fine chemical 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose its function as a probe extends to the detection of microorganisms, plant tissues, plants, plant variets, plant ecotypes or plant genera with varying capability or potential for synthesis of the respective fine chemical 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose. Therefore in one embodiment the present invention relates to a method for analyzing the capability or potential of a plant tissue, a plant, a plant variety or plant ecotype to produce the fine chemical 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose by using the nucleic acid of the invention or parts thereof as a probe to detect the amount of the nucleic acid of the invention in the non-human organism or a part thereof in comparison to another non-human organism.

The nucleic acid molecule of the invention encodes a polypeptide or portion thereof which includes an amino acid sequence which is sufficiently homologous to the amino acid sequence shown in respective line in Table II, application no. 17, columns 5 or 8 such that the protein or portion thereof maintains the ability to confer the production or the increased production of the respective fine chemical 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose as compared to a corresponding, e.g. non-transformed, wild type non-human organism, like a microorganism or a plant cell, plant or part thereof, in particular increasing the activity as mentioned above or as described in the examples in microorganisms or plants.

As used herein, the language “sufficiently homologous” refers to proteins or portions thereof which have amino acid sequences which include a minimum number of identical or equivalent amino acids (e.g. an amino acid which has a similar side chain as an amino acid in one of the sequences of the polypeptide of the present invention) to an amino acid sequence shown in the respective line in Table II, application no. 17, columns 5 or 8 such that the protein or portion thereof is able to confer the production or the increased production of the respective fine chemical 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose as compared to a corresponding, e.g. non-transformed, wild type non-human organism, like a microorganism or a plant cell, plant or part thereof. For example having the activity of a protein as shown in the respective line in Table II, application no. 17, column 3 and as described herein.

In an embodiment of the present invention, the nucleic acid molecule of the present invention comprises a nucleic acid molecule that encodes a portion of the protein of the present invention. The protein is at least about 30%, 35%, 40%, 45% 50%, 55%, 60%, 65%, 70%, 75%, 80%, 85%, 90%, 91%, 92%, 93%, 94%, 95%, 96%, 97%, 98%, 99%, 99.5% or more homologous to an entire amino acid sequence shown in the respective line of Table II, application no. 17, column 5 or 8 and has the above-mentioned activity, e.g. conferring the production or the increased production of the respective fine chemical 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose as compared to a corresponding, e.g. non-transformed, wild type non-human organism, like a microorganism or a plant cell, plant or part thereof by, for example in a embodiment expression either in the cytosol or in an organelle such as a plastid or mitochondria or both, preferably in a plastid, or in another embodiment by targeted or non-targeted expression

for the disclosure of this paragraph see [0338.1.7.7] above

for the disclosure of this paragraph see [0339.1.7.7] above

The invention further relates to nucleic acid molecules or methods using said nucleic acid molecules that differ from one of the nucleotide sequences shown in the respective line in Table I, application no. 17, columns 5 or 8, preferably the coding region thereof, or fragments or homologs thereof, due to degeneracy of the genetic code but encode a polypeptide of the present invention, in particular a polypeptide having the above-mentioned activity, e.g. as those polypeptides depicted in the respective line in Table II, application no. 17, columns 5 or 8, fragments or homologs thereof. Advantageously, the nucleic acid molecule of the invention comprises, or in an other embodiment has, a nucleotide sequence encoding a protein comprising, or in an other embodiment having, an amino acid sequence shown in the respective line in Table II, application no. 17, columns 5 or 8, fragments or homologs thereof. In a further embodiment, the nucleic acid molecule of the invention encodes a full length protein which is substantially homologous to an amino acid sequence shown in the respective line in Table II, application no. 17, columns 5 or 8, or homologs thereof. However, in an embodiment, the nucleic acid molecule of the present invention does not consist of the sequence shown in the respective line in Table I, preferably Table IA, application no. 17, columns 5 or 8, preferably the coding region thereof, but the functional homologs thereof.

[0341.1.7.17] to [0343.1.7.17] for the disclosure of these paragraphs see [0341.1.7.7] to [0343.1.7.7] above

Accordingly, in another embodiment, a nucleic acid molecule of the invention is at least 15, 20, 25 or 30 nucleotides in length. Preferably, it hybridizes under stringent conditions to a nucleic acid molecule comprising a nucleotide sequence of the nucleic acid molecule of the present invention or used in the process of the present invention, e.g. comprising the sequence shown in the respective line in Table I, application no. 17, columns 5 or 8, preferably the coding region thereof. In another embodiment the nucleic acid molecule is preferably at least 20, 30, 50, 100, 250 or more nucleotides in length.

for the disclosure of this paragraph see [0345.1.7.7] above

Preferably, a nucleic acid molecule of the invention that hybridizes under stringent conditions to a sequence shown in the respective line in Table I, application no. 17, columns 5 or 8, preferably the coding region thereof, corresponds to a naturally-occurring nucleic acid molecule of the invention. As used herein, a “naturally-occurring” nucleic acid molecule refers to a RNA or DNA molecule having a nucleotide sequence that occurs in nature (e.g.

encodes a natural protein). Preferably, the nucleic acid molecule encodes a natural protein having above-mentioned activity, e.g. conferring the production or the increased production of the respective fine chemical 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose as compared to a corresponding, e.g. non-transformed, wild type non-human organism, like a microorganism or a plant cell, plant or part thereof after increasing the expression or activity thereof or the activity of a protein of the invention or used in the process of the invention, in an embodiment for example expression either in the cytosol or in an organelle such as a plastid or mitochondria, preferably in plastids, or, in another embodiment by targeted or non-targeted expression.

for the disclosure of this paragraph see [0347.1.7.7] above

For example, nucleotide substitutions leading to amino acid substitutions at “non-essential” amino acid residues can be made in a sequence of the nucleic acid molecule of the invention or used in the process of the invention, e.g. shown in the respective line in Table I, application no. 17, columns 5 or 8, preferably the coding region thereof, or fragments or homologs thereof.

for the disclosure of this paragraph see [0349.1.7.7] above

for the disclosure of this paragraph see [0350.1.7.7] above

Accordingly, the invention relates to nucleic acid molecules encoding a polypeptide having above-mentioned activity, in a non-human organisms or parts thereof by for example expression either in the cytosol or in an organelle such as a plastid or mitochondria or both, preferably in plastids, that contain changes in amino acid residues that are not essential for said activity. Such polypeptides differ in amino acid sequence from a sequence depicted in the respective line in Table II, especially Table IIA, application no. 17, columns 5 or 8, but retain said activity described herein. The nucleic acid molecule can comprise a nucleotide sequence encoding a polypeptide, wherein the polypeptide comprises an amino acid sequence at least about 50% identical to an amino acid sequence shown in the respective line in Table II, application no. 17, columns 5 or 8, and is capable of conferring the production or the increased production of the respective fine chemical 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose as compared to a corresponding, e.g. non-transformed, wild type non-human organism, like a microorganism or a plant cell, plant or part thereof, after increasing its activity for example in an embodiment by expression either in the cytosol or in an organelle such as a plastid or mitochondria or both, preferably in plastids, or, in another embodiment by targeted or non-targeted expression. Preferably, the protein encoded by the nucleic acid molecule is at least about 60%, 70%, 80%, 90%, 95%, 96%, 97%, 98%, 99% or 99.5% identical to the sequence shown in the respective line in Table II, application no. 17, columns 5 or 8.

[0352.1.7.17] to [0343.1.7.17] for the disclosure of these paragraphs see [0352.1.7.7] to [0343.1.7.7] above

Functional equivalents of the nucleic acid molecules according to the present invention and/or used in the process according to the present invention represent an embodiment of homologs. Functional equivalents derived from the nucleic acid molecules as shown in the respective line in Table I, application no. 17, columns 5 or 8, preferably the coding region thereof, according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the nucleic acid molecules as shown in the respective line in Table I, application no. 17, columns 5 or 8, preferably the coding region thereof, according to the invention and encode polypeptides having essentially the same properties as the polypeptide as shown in the respective line in Table II, application no. 17, columns 5 or 8.

Functional equivalents derived from one of the polypeptides as shown in the respective line in Table II, application no. 17, columns 5 or 8 according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the polypeptides as shown in the respective line in Table II, application no. 17, columns 5 or 8 according to the invention and having essentially the same properties as the polypeptide as shown in the respective line in Table II, application no. 17, columns 5 or 8.

for the disclosure of this paragraph see [0359.1.7.7] above

A nucleic acid molecule encoding a homolog to a polypeptide depicted in the respective line in Table II, application no. 17, columns 5 or 8 can be created by introducing one or more nucleotide substitutions, additions or deletions into the nucleic acid molecule of the present invention, in particular of Table I, application no. 17, columns 5 or 8, in the respective line, such that one or more amino acid substitutions, additions or deletions are introduced into the encoded protein. Mutations can be introduced into the nucleic acid molecules as depicted in the respective line in Table I, application no. 17, columns 5 or 8, preferably the coding region thereof, by standard techniques, such as site-directed mutagenesis, PCR-mediated mutagenesis or other methods known to the person skilled in the art.

[0361.1.7.17] to [0363.1.7.17] for the disclosure of these paragraphs see [0361.1.7.7] to [0363.1.7.7] above

Homologues of the nucleic acid sequences used, with the sequences shown in the respective line in Table I, application no. 17, columns 5 or 8, preferably the coding region thereof, comprise also allelic variants with at least approximately 30%, 35%, 40%, 45%, 50%, 55%, 60%, 65%, 70%, 75%, 80%, 85%, 90%, 91%, 92%, 93%, 94% 95%. 96%, 97%, 98%, 99%, 99.5% or more % homology with one of the nucleotide sequences shown or the above-mentioned derived nucleic acid sequences or their homologues or derivatives or parts of these. Allelic variants encompass in particular functional variants which can be obtained by deletion, insertion or substitution of nucleotides from the sequences shown in the respective line, preferably in Table I, columns 5 or 8, preferably the coding region thereof, or from the derived nucleic acid sequences, the intention being, however, that the enzymatic activity or the biological activity of the resulting proteins synthesized is advantageously retained or increased.

In one embodiment of the present invention, the nucleic acid molecule of the invention or used in the process of the invention comprises the sequences shown in the respective line in any of the Table I, application no. 17, columns 5 or 8, preferably the coding region thereof, fragments or homologs thereof. It is preferred that the nucleic acid molecule comprises as little as possible other nucleotides not shown in the respective line in any one of Table I, application no. 17, columns 5 or 8, preferably the coding region thereof, fragments or homologs thereof. In one embodiment, the nucleic acid molecule comprises less than 500, 400, 300, 200, 100, 90, 80, 70, 60, 50 or 40 further nucleotides. In a further embodiment, the nucleic acid molecule comprises less than 30, 20 or 10 further nucleotides. In one embodiment, the nucleic acid molecule used in the process of the invention is identical to the sequences shown in the respective line in Table I, application no. 17, columns 5 or 8, preferably the coding region thereof.

Also preferred is that the nucleic acid molecule used in the process of the invention encodes a polypeptide comprising the sequence shown in the respective line in Table II, application no. 17, columns 5 or 8, fragments or homologs thereof. In one embodiment, the nucleic acid molecule encodes less than 150, 130, 100, 80, 60, 50, 40 or 30 further amino acids. In a further embodiment, the encoded polypeptide comprises less than 20, 15, 10, 9, 8, 7, 6 or 5 further amino acids. In one embodiment used in the inventive process, the encoded polypeptide is identical to the sequences shown in the respective line in Table II, application no. 17, columns 5 or 8.

In one embodiment, the nucleic acid molecule of the invention or used in the process encodes a polypeptide comprising the sequence shown in the respective line in Table II, application no. 17, columns 5 or 8, or fragments or homologs thereof, and comprises less than 100 further nucleotides. In a further embodiment, said nucleic acid molecule comprises less than 30 further nucleotides. In one embodiment, the nucleic acid molecule used in the process is identical to a coding sequence of the sequences shown in the respective line in Table I, application no. 17, columns 5 or 8.

Polypeptides (=proteins), which still have the essential biological or enzymatic activity of the polypeptide of the present invention conferring the production or the increased production of the respective fine chemical 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose as compared to a corresponding, e.g. non-transformed, wild type non-human organism, like a microorganism or a plant cell, plant or part thereof, i.e. whose activity is essentially not reduced, are polypeptides with at least 10%, 20%, 30%, 40%, 50%, 60%, 70%, 80%, 90%, 95% or more of the wild type biological activity or enzymatic activity, advantageously, the activity is essentially not reduced in comparison with the activity of a polypeptide shown in the respective line in Table II, application no. 17, columns 5 or 8 expressed under identical conditions.

Homologues of nucleic acid molecules shown in the respective line in Table I, application no. 17, columns 5 or 8, preferably the coding region thereof, or fragments thereof, or of the derived sequences shown in the respective line in Table II, application no. 17, columns 5 or 8, or fragments thereof, also mean truncated sequences, cDNA, single-stranded DNA or RNA of the coding and noncoding DNA sequence. Homologues of said sequences are also understood as meaning derivatives, which comprise noncoding regions such as, for example, UTRs, introns, terminators, enhancers or promoter variants. The promoters upstream of the nucleotide sequences stated can be modified by one or more nucleotide substitution(s), insertion(s) and/or deletion(s) without, however, interfering with the functionality or activity either of the promoters, the open reading frame (=ORF) or with the 3′-regulatory region such as terminators or other 3′-regulatory regions, which are far away from the ORF. It is furthermore possible that the activity of the promoters is increased by modification of their sequence, or that they are replaced completely by more active promoters, even promoters from heterologous organisms. Appropriate promoters are known to the person skilled in the art and are mentioned herein below.

[0370.1.7.17] to [0379.1.7.17] for the disclosure of these paragraphs see [0370.1.7.7] to [0379.1.7.7] above

Moreover, a native polypeptide conferring the increase of the respective fine chemical 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose in a non-human organism or a part thereof can be isolated from cells (e.g., endothelial cells), for example using the antibody of the present invention as described below, in particular, an antibody against proteins having 1,4-alpha-glucan branching enzyme, 2,3-dihydroxy-2,3-dihydrophenylpropionatedehydrogenase, 2-C-methyl-D-erythritol 4-phosphate cytidylyltransferase, 2-oxoglutarate dehydrogenase E1 subunit, 3-deoxy-7-phosphoheptulonate synthase, 3isopropylmalate dehydratase large subunit, 3-phosphoglycerate dehydrogenase, 47266012-protein, 49747384_SOYBEAN-protein, 4-alpha-glucanotransferase, ABC transporter permease protein, acetyl CoA carboxylase, acetyl CoA synthetase, acetyl-coenzyme A synthetase, acetyltransferase, acid shock protein, acyl carrier protein, acyl transferase, acyl-CoA dehydrogenase, acyl-CoA synthase, adenylosuccinate lyase, alanine dehydrogenase, alcohol dehydrogenase, aldolase, alkaline phosphatase, amine oxidase, amino-acid acetyltransferase, aminotransferase, arginine exporter protein, asparagine synthetase B, AO g19350-protein, At2g45420-protein, At5g16650-protein, At5g43630-protein, ATP synthase subunit E, ATP-binding component of a transport system, auxin response factor, AX653549-protein, AY087308-protein, b0050-protein, b0251-protein, b0362-protein, b0488-protein, B0644-protein, b0801-protein, b0970-protein, b1003-protein, b1046-protein, b1137-protein, b1214-protein, b1219-protein, b1234-protein, b1285-protein, b1357-protein, b1410-protein, b1425-protein, b1445-protein, b1470-protein, b1522-protein, b1556-protein, b1583-protein, b1670-protein, b1796-protein, b1898-protein, b2032-protein, b2107-protein, b2139-protein, b2345-protein, b2360-protein, b2361-protein, b2399-protein, b2466-protein, b2474-protein, b2613-protein, b2653-protein, b2654-protein, b2670-protein, b2673-protein, b2682-protein, b2739-protein, b2932-protein, b3151-protein, b3221-protein, b3246-protein, b3346-protein, B3427-protein, b3442-protein, B3443-protein, b3777-protein, b3817-protein, b3818-protein, b3989-protein, b4029-protein, b4050-protein, b4056-protein, b4250-protein, beta-galactosidase, beta-hydroxylase, betaine aldehyde dehydrogenase, bifunctional protein (phosphoribosyltransferase and regulatory protein), binding-protein-dependent transport systems inner membrane component, branched-chain amino acid ABC transporter permease protein, branched-chain amino acid permease, calcium-dependent protein kinase, carbamoyl-phosphate synthase subunit, cardiolipin synthase, cation-transporting ATPase, CBL-interacting protein kinase, CCAAT-binding transcription factor, CDP-diglyceride synthetase, cell division control protein, cell division protein, chlorophyllase, chorismate lyase, circadian clock protein, colanic acid biosynthesis protein, coproporphyrinogen III oxidase, coproporphyrinogen oxidase, CTP synthase, cullin, cyclin, cyclin D, cystathionine gamma-synthase, cysteine synthase A, dATP pyrophosphohydrolase, dihydrolipoamide dehydrogenase, DNA binding protein, DNA replication complex GINS protein, DnaJ-like chaperone, electron transfer flavoprotein subunit beta, electron transport complex protein, elongation factor Tu, enoyl-CoA hydratase, eukaryotic translation initiation factor, eukaryotic translation initiation factor 5, fatty acid desaturase, ferredoxin, Fe—S subunit of oxidoreductase, flagellar M-ring protein, flavodoxin, fructose biphosphate aldolase, fumarylacetoacetate hydrolase, galactinol synthase, GDP-mannose 4,6-dehydratase, GDP-mannose dehydrogenase, geranylgeranyl pyrophosphate synthase, gibberellin 20-oxidase, Glu/Leu/Phe/Val dehydrogenase, glucokinase, gluconate transport system permease 3, glucose dehydrogenase, glucose-6-phosphate 1-dehydrogenase, glutamate-ammonia-ligase, glutamine amidotransferase, glutamine synthetase, glutaredoxin, glycerate kinase, glycogen (starch) synthase, glycogen synthase, glycosidase, glycosyl transferase, glycosyltransferase, GM02LC38418-protein, GM02LC46-protein, halotolerance protein, heat shock transcription factor, hexokinase, hexose transporter, high-affinity branched-chain amino acid transport protein, histone acetyltransferase, homocitrate synthase, hydrolase, inositol-3-phosphate synthase, integral membrane protein, intracellular septation protein, ion-transport protein, iron(III) dicitrate-binding protein, isochorismate synthase, isocitrate dehydrogenase, isopropylmalate isomerase large subunit, ketol-acid reductoisomerase, kinase, leucyl/phenylalanyl-tRNA-protein transferase, lipoprotein precursor, L-ribulose-5-phosphate 4-epimerase, L-serine dehydratase, lysine decarboxylase, MADS box protein transcription factor, MADS-box transcription factor, malate dehydrogenase, mannitol-1-phosphate dehydrogenase, membrane protein, membrane transport protein, methylglyoxalase, methyltransferase, Mg-protoporphyrin IX monomethyl ester oxidative cyclase 66 kD subunit, mitogen-activated protein kinase, molecular chaperone, monothiol glutaredoxin, monthiol glutaredoxin, multidrug resistance protein, multiple antibiotic resistance protein, murein transglycosylase, NAD(P)H dehydrogenase (quinone), NADH dehydrogenase, NADH-quinone oxidoreductase subunit, O-acetyltransferase, O-antigen chain length determinant, outer membrane receptor protein precursor, oxidoreductase, oxidoreductase subunit, para-aminobenzoate synthase component I, penicillin-binding protein, peptidyl-prolyl cis-trans isomerase, permease, permease protein of ABC transporter, phage-related repressor protein, phenylacetic acid degradation protein, phenylalanine ammonia-lyase, phosphate acetyltransferase, phosphate permease, phosphatidylinositol 3- and 4-kinase family protein, phosphoadenosine phosphosulfate reductase, phosphoanhydride phosphorylase, phosphoenolpyruvate carboxykinase, phosphoglycerate kinase, phosphohydrolase, phosphotransferase system component, photosystem I reaction center subunit XI, photosystem I reaction centre subunit, photosystem II protein, polyamine transporter, precorrin methylase, protease, protein kinase, protein phosphatase, protein-L-isoaspartate O-methyltransferase, purine biosynthesis protein, purine nucleoside phosphorylase, purine-nucleoside phosphorylase, putative transport system permease protein, putrescine transport system permease protein, pyridoxal phosphate biosynthetic protein, pyrimidine-specific ribonucleoside hydrolase, pyruvate kinase, recombinase A, RNA-binding protein, sec-independent protein translocase, Sec-independent protein translocase subunit, sensor histidine kinase, serine protease, serine/threonine dehydratase, serine/threonine-protein phosphatase, short chain dehydrogenase, short-chain alcohol dehydrogenase family, sll0254-protein, sll0281-protein, sll0354-protein, sll0418-protein, sll1761-protein, slr0600-protein, slr1107-protein, sn-glycerol-3-phosphate dehydrogenase, sn-glycerol-3-phosphate transport system permease protein, sodium/proton antiporter, sterol O-acyltransferase, subunit of COMPASS-protein complex, succinyl-CoA synthetase beta chain, ThiF family protein, thioredoxin, thioredoxin family protein, threonine dehydratase, threonine efflux protein, threonine synthase, transcription factor, transcriptional regulator, transcriptional regulator protein, transport protein, trehalase, trehalose-6-phosphate synthase, trehalose-phosphatase, tryptophan biosynthesis protein, TTC1386-protein, urease accessory protein, urease subunit, vacuolar sorting protein, XM_{—}473199-protein, ycl027w-protein, YCR047W-A-protein, ycr102c-protein, YDL159W-A-protein, ydl235c-protein, ydr183w-protein, ydr507c-protein, YEL030C-A-protein, yer177w-protein, YFL019C-protein, ygl096w-protein, ygl237c-protein, yhl013c-protein, YIL083C-protein, YJL127W-A-protein, ymr324c-protein, yol160w-protein, ypl144w-protein, ypr098c-protein, zinc transporter, or Zm_{—}4842_BE510522-protein activity, respectively, or an antibody against poly peptides as shown in the respective line in Table II, application no. 17, columns 5 or 8, or fragments or homologs thereof which can be produced by standard techniques utilizing the polypeptid of the present invention or fragment thereof, i.e., the polypeptide of this invention (FCRP). Preferred are monoclonal antibodies.

for the disclosure of this paragraph see [0381.1.7.7] above

In one embodiment, the present invention relates to a polypeptide as depicted in the respective line in Table II, especially in Table IIA or especially in Table IIB, application no. 17, columns 5 or 8, or fragment or homolog thereof or as coded by the nucleic acid molecule shown in the respective line in Table I, especially Table IA or especially Table IB, application no. 17, columns 5 or 8, preferably the coding region thereof, or fragment or homolog thereof.

In an embodiment, the present invention relates to a polypeptide comprising or consisting of a consensus sequence shown in the respective line in Table IV, application no. 17, column 8 or at least one polypeptide motif shown in the respective line in Table IV, application no. 17, column 8.

In an embodiment thereof, the present invention relates to a polypeptide comprising or consisting of a consensus sequence shown in the respective line in Table IV, application no. 17, column 8 or comprising at least one polypeptide motif shown in the respective line in Table IV, application no. 17, column 8, whereby not more than 20, 15, 10, 9, 8, 7, 6, 5, 4, 3, 2, or 1, or 0 of the amino acids can be replaced by any amino acid.

In another embodiment thereof, the present invention relates to a polypeptide comprising or consisting of a consensus sequence shown in the respective line in Table IV, application no. 17, column 8 or comprising at least one polypeptide motif shown in the respective line in Table IV, application no. 17, column 8, whereby not more than 15%, 10%, 5%, 4%, 3%, 2% or1% or 0% of the amino acids defined as distinct specific amino acids are/is replaced by another amino acid.

In another embodiment thereof, the present invention relates to a polypeptide comprising or consisting of a consensus sequence shown in the respective line in Table IV, application no. 17, column 8 or comprising at least one polypeptide motif shown in the respective line in Table IV, application no. 17, column 8, whereby not more than 20, 15, 10, 9, 8, 7, 6, 5, 4, 3, 2, or 1, or 0 amino acids are inserted into the consensus sequence or polypeptide motif.

In an embodiment of the present invention, the polypeptide of the present invention comprises or consists of a consensus sequence or at least one polypeptide motif shown in the respective line in Table IV, application no. 17, column 8, whereby said polypeptide distinguishes over the sequence depicted in the respective line in Table II, application no. 17, columns 5 or 8 by one or more amino acids. In one embodiment, said polypeptide of the invention distinguishes over the sequence shown in the respective line in Table II, application no. 17, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 17, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in the respective line in Table II, application no. 17, columns 5 and 8.

In an embodiment, the invention relates to a polypeptide conferring an increase in the respective fine chemical 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose in a non-human organism, especially a microorganism or a plant, or a part thereof, being encoded by the nucleic acid molecule of the invention or used in the process of the invention and having a sequence which distinguishes over the sequence as shown in the respective line in Table II, application no. 17, columns 5 or 8 by one or more amino acids (but not exceeding 5%, preferably 4%, 3%, 2%, 1%, 0.5%, 0.2% or 0.1%). In an embodiment, said polypeptide of the invention does not comprise or consist of the sequence shown in the respective line in Table II, application no. 17, columns 5 or 8. In an embodiment, said polypeptide of the present invention is less than 100%, 99.999%, 99.99%, 99.9% or 99% identical. In one embodiment, said polypeptide which differs at least in one or more amino acids (but not exceeding 5%, preferably 4%, 3%, 2%, 1%, 0.5%, 0.2% or 0.1%) from the polypeptide shown in the respective line in Table II, application no. 17, columns 5 and 8 does not comprise a protein of the sequence shown in the respective line in Table II A and/or II B, application no. 17, columns 5 or 8.

In an embodiment, the present invention relates to a polypeptide having the activity of the protein as shown in the respective line in Table II, application no. 17, column 3, which distinguishes over the sequence depicted in the respective line in Table II, application no. 17, columns 5 or 8 by one or more amino acids, preferably by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 17, columns 5 and 8 by not more than 80%, 70%, 60%, 50%, 40%, 30%, 20%, 10%, 5%, 4%, 3%, 2%, 1% or 0.5%. In another embodiment the polypeptide of the invention takes the form of a preprotein consisting of a plastidial or mitochondrial transit peptide joint to a polypeptide having the activity of the protein as shown in Table II, column 3, from which the transit peptide is preferably cleaved off upon transport of the preprotein into the organelle for example into the plastid or mitochondrion.

In another embodiment the polypeptide of the invention takes not the form of a preprotein.

for the disclosure of this paragraph see [0390.1.7.7] above

for the disclosure of this paragraph see [0391.1.7.7] above

A polypeptide of the invention can participate in the process of the present invention. The polypeptide or a portion thereof comprises preferably an amino acid sequence, which is sufficiently homologous to an amino acid sequence shown in the respective line in Table II A and/or II B, application no. 17, columns 5 or 8.

Further, the polypeptide can have an amino acid sequence which is encoded by a nucleotide sequence which hybridizes, preferably hybridizes under stringent conditions as described above, to a nucleotide sequence of the nucleic acid molecule of the present invention. Accordingly, the polypeptide has an amino acid sequence which is encoded by a nucleotide sequence that is at least about 35%, 40%, 45%, 50%, 55%, 60%, 65%, 70%, 75%, 80%, 85%, 90%, 91%, 92%, 93%, 94%, 95%, 96%, 97%, 98%, 99%, 99.5% or more homologous to one of the amino acid sequences as shown in the respective line in Table II A and/or II B, application no. 17, columns 5 or 8, or fragments thereof. The preferred polypeptide of the present invention preferably possesses at least one of the activities according to the invention and described herein. A preferred polypeptide of the present invention includes an amino acid sequence encoded by a nucleotide sequence which hybridizes, preferably hybridizes under stringent conditions, to a nucleotide sequence shown in the respective line in Table I A and/or I B, application no. 17, columns 5 or 8, preferably the coding region thereof, or fragments thereto or which is homologous thereto, as defined above

The polypeptide of the present invention can vary from the sequences shown in the respective line in Table II A and/or II B, application no. 17, columns 5 or 8 in amino acid sequence due to natural variation or mutagenesis, as described in detail herein. Accordingly, the polypeptide comprise an amino acid sequence which is at least about 35%, 40%, 45%, 50%, 55%, 60%, 65%, 70%, 75%, 80%, 85% or 90, 91%, 92%, 93%, 94%, 95%, 96%, 97%, 98%, 99%, 99.5% or more homologous to an entire amino acid sequence shown in the respective line in Table II A and/or II B, application no. 17, columns 5 or 8.

Biologically active portions of a polypeptide of the present invention include peptides comprising amino acid sequences derived from the amino acid sequence of the polypeptide of the present invention or used in the process of the present invention, e.g., the amino acid sequence shown in the respective line in Table II, application no. 17, columns 5 or 8 or the amino acid sequence of a protein homologous thereto, which include fewer amino acids than a full length polypeptide of the present invention or used in the process of the present invention or the full length protein which is homologous to a polypeptide of the present invention or used in the process of the present invention depicted herein, and exhibit at least one activity of a polypeptide of the present invention or used in the process of the present invention.

for the disclosure of this paragraph see [0396.1.7.7] above

Manipulation of the nucleic acid molecule of the invention may result in the production of a protein having differences from the protein as shown in the respective line in Table II, application no. 17, column 5 or 8. Differences shall mean at least one amino acid different from the sequences as shown in the respective line in Table II, application no. 17, column 5 or 8, preferably at least 2, 3, 4, 5, 6, 7, 8, 9 or 10 amino acids (especially but not exceeding 0.1%, preferably 0.2%, 0.5%, 1%, 2%, 3% 4% or 5%), more preferably at least 15, 20, 25, 30, 35, 40, 45 or 50 amino acids (especially but not exceeding 0.5%, preferably 1%, 2%, 3% 4%, 5%, 10% or 25%) different from the sequences as shown in the respective line in Table II, application no. 17, column 5 or 8. These proteins may be improved in efficiency or activity, or may be more stable and therefore present in greater numbers in the cell as compared to the wild-type cell.

Any mutagenesis strategies for the polypeptide of the present invention or the polypeptide used in the process of the present invention to result in increasing said activity are not meant to be limiting; variations on these strategies will be readily apparent to one skilled in the art. Using such strategies, and incorporating the mechanisms disclosed herein, the nucleic acid molecule and polypeptide of the invention may be utilized to generate algae, ciliates, fungi, other microorganisms like C. glutamicum or plants or parts thereof, expressing proteins as shown in the respective line in Table II, application no. 17, column 5 or 8, or mutated proteins thereof. The nucleic acid molecules and polypeptide molecules of the invention are expressed such that the yield, production, and/or efficiency of production of a desired compound is improved.

[0399.1.7.17] to [0401.1.7.17] for the disclosure of these paragraphs see [0399.1.7.7] to [0401.1.7.7] above

In one embodiment, a protein (=polypeptide) as shown in the respective line in Table II, application no. 17, column 5 or 8 refers to a polypeptide having an amino acid sequence corresponding to the polypeptide of the invention, especially to a polypeptide as shown in Table II, application no. 17, columns 5 or 8 or homologs thereof, or used in the process of the invention, whereas a “non-inventive protein or polypeptide” or “other polypeptide” refers to a polypeptide having an amino acid sequence corresponding to a protein which is not substantially homologous to a polypeptide of the invention, preferably which is not substantially homologous to a polypeptide or protein as shown in the respective line in Table II, application no. 17, column 5 or 8 or homologs thereof, e.g., a protein which does not confer the activity described herein and which is derived from the same or a different organism.

[0403.1.7.17] to [0409.1.7.17] for the disclosure of these paragraphs see [0403.1.7.7] to [0409.1.7.7] above

As the nucleic acid molecules of the invention and/or the expression thereof is related to the synthesis of the respective fine chemical 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose its function as a probe extends to the detection of microorganisms, plant tissues, plants, plant variets, plant ecotypes or plant genera with varying, advantageously increased, capability or potential for synthesis of the respective fine chemical 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose. Therefore in one embodiment the present invention relates to a method for analyzing the capability or potential of a plant tissue, a plant, a plant variety or ecotype to produce the respective fine chemical 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose by using the respective antibody of the invention as a probe to detect the amount of the polypeptide encoded by said nucleic acid molecule of the invention in a non-human organism in comparison to another organism.

Therefore in one embodiment the invention relates to a method to probe a non-human organism for its capability for the production of the respective fine chemical comprising the steps of
a) analyzing the sequence and/or the expression of the respective nucleic acid molecule of the invention in least two non-human organisms which differ in the capability to produce the respective fine chemical;
b) associate the different sequence and/or expression level of the nucleic acid molecules of the invention with their different capability to produce the respective fine chemical;
c) analyze the sequence and/or the expression of the nucleic acid molecule of the invention in at least a third non-human organism;
d) predict the capability of the at least third organism to produce the respective fine chemical based by comparing the sequence and/or expression of the nucleic acid of the invention to the sequences and/or expression levels of the nucleic acid molecule of the invention of the at least two organisms analyzed in step a.

[0411.1.7.17] to [0430.1.7.17] for the disclosure of these paragraphs see [0411.1.7.7] to [0430.1.7.7] above

Accordingly the present invention relates to any cell transgenic for any nucleic acid characterized as part of the invention, e.g. conferring the increase of the respective fine chemical 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose in a cell or a non-human organism or a part thereof, e.g. the nucleic acid molecule of the invention, the nucleic acid construct of the invention, the vector of the invention, the expression cassette according to the invention, or a nucleic acid molecule encoding the polypeptide of the invention, e.g. encoding a polypeptide having an activity as the protein as shown in the respective line in Table II, application no. 17, column 3. Due to the above-mentioned activity the respective fine chemical 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose content in a cell or a non-human organism is increased. For example, due to modulation or manipulation, the cellular activity is increased, in a preferred embodiment in organelles such as plastids or mitochondria, e.g. due to an increased expression or specific activity or specific targeting of the subject matters of the invention in a cell or a non-human organism or a part thereof especially in organelles such as plastids or mitochondria, or in another embodiment in the cytosol. Transgenic for a polypeptide having a protein or a protein activity means herein that due to modulation or manipulation of the genome, the activity of protein as shown in the respective line in Table II, application no. 17, column 3 or a protein as shown in the respective line in Table II, application no. 17, column 3like activity is increased in the cell or non-human organism or part thereof, especially in organelles such as plastids or mitochondria, or especially in the cytosol. Examples are described above in context with the process of the invention.

for the disclosure of this paragraph see [0432.1.7.7] above

A naturally occurring expression cassette—for example the naturally occurring combination of the promoter of the gene encoding a protein as shown in the respective line in Table II, application no. 17, column 5 or 8, preferably the coding region thereof, or fragments or homologs thereof with the corresponding encoding gene—becomes a transgenic expression cassette when it is modified by non-natural, synthetic “artificial” methods such as, for example, mutagenesis. Such methods have been described (U.S. Pat. No. 5,565,350; WO 00/15815; also see above).

for the disclosure of this paragraph see [0434.1.7.7] above

for the disclosure of this paragraph see [0435.1.7.7] above

Transgenic plants comprising the respective fine chemical(s) synthesized in the process according to the invention can be marketed directly without isolation of the fine chemical synthesized. In the process according to the invention, plants are understood as meaning all plant parts, plant organs such as leaf, stalk, root, tubers or seeds or propagation material or harvested material or the intact plant. In this context, the seed encompasses all parts of the seed such as the seed coats, epidermal cells, seed cells, endosperm or embryonic tissue. The fine chemical produced in the process according to the invention may, however, also be isolated from the plant. In case of 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, or verbascose this can be in free form or bound to proteins. Fine chemical(s) produced by this process can be harvested by harvesting the non-human organisms either from the culture in which they grow or from the field. For example, this can be done via squeezing, grinding and/or extraction, salt precipitation and/or ion-exchange chromatography of the plant parts, preferably the plant seeds, plant fruits, plant tubers and the like.

[0437.1.7.17] to [0447.1.7.17] for the disclosure of these paragraphs see [0437.1.7.7] to [0447.1.7.7] above

The identity and purity of the isolated compound(s) can be determined by prior art techniques. These include high performance liquid chromatography (HPLC), spectroscopic methods, mass spectrometry (MS), staining methods, thin-layer chromatography, NIRS, enzyme assay or microbiological assays. These analytical methods are summarized for example in: Patek et al., Appl. Environ. Microbiol. 60, 133 (1994), Malakhova et al., Biotekhnologiya 11, 27 (1996); Schmidt et al., Bioprocess Engineer. 19, 67 (1998), Ullmann's Encyclopedia of Industrial Chemistry (1996) Vol. A27, VCH, Weinheim, pp. 89-90, pp. 521-540, pp. 540-547, pp. 559566, 575-581 and pp. 581-587, Michal G., “Biochemical Pathways: An Atlas of Biochemistry and

Molecular Biology”, John Wiley and Sons (1999); Fallon A. et al. “Applications of HPLC in Biochemistry” in “Laboratory Techniques in Biochemistry and Molecular Biology”, Vol. 17 (1987). The fine chemical of the invention can for example be analyzed advantageously via HPLC or GC separation methods and detected by MS or MSMS methods. The unambiguous detection for the presence of the fine chemical containing products can be obtained by analyzing recombinant organisms using analytical standard methods: GC, GC-MS or TLC, as described on several occasions by Christie and the references therein (1997, in: Advances on Lipid Methodology, Fourth Edition: Christie, Oily Press, Dundee, 119-169; 1998, GaschromatographieMassenspektrometrie-Verfahren [Gas chromatography/mass spectrometric methods], Lipide 33:343-353). The material to be analyzed can be disrupted by sonication, grinding in a glass mill, liquid nitrogen and grinding, cooking, or via other applicable methods; see also Biotechnology of Vitamins, Pigments and Growth Factors, Edited by Erik J. Vandamme, London, 1989, p.96 to 103.

The fine chemical obtained in the process are suitable as starting material for the synthesis of further products of value. For example, in case of vitamins they can be used in combination with each other or alone for the production of pharmaceuticals, foodstuffs, animal feeds or cosmetics.

Accordingly, the present invention relates to a method for the production of pharmaceuticals, food stuff, animal feeds, nutrients or cosmetics comprising the steps of the process according to the invention, including the isolation of the saccharides composition produced or the produced fine chemical carbohydrate, e.g. monosaccharides, preferably fructose and/or glucose, or other pentoses or hexoses; in free or bound form, preferably glucose-1-phosphate and/or sedoheptulose-7-phosphate; and/or derivates of monosaccharides, preferably (sugar) acids, e.g. saccharic acid, 3,4,5-trihydroxypentanoic acid; and/or sugar alcohols e.g. erythrol, galactitol, sorbitol, mannitol and/or myo-inositol; and/or deoxy-derivates, e.g. fucose; and/or disaccharides, preferably sucrose, melibiose, galactinol and/or trehalose, and/or oligosaccharides, preferably the

RFOs (raffinose family of oligosaccharides: preferably the trisaccharide raffinose and/or the pentasaccharide verbascose), and/or polysaccharides, preferably starch and/or cellulose if desired and formulating the product with a pharmaceutical or cosmetic acceptable carrier or formulating the product in a form acceptable for an application in agriculture. A further embodiment according to the invention is the use of the sugars, preferably fructose produced in the process or of the transgenic organisms in animal feeds, foodstuffs, medicines, food supplements, cosmetics or pharmaceuticals.

In one embodiment of the invention erytrhol is used as growth-accelerating factor on plants and microorganisms and/or as component of toothpaste.

In one embodiment of the invention mannitol and/or sorbitol is used as sugar substitute.

In one embodiment of the invention D-sedoheptulose-7-phosphate is used synthesis of the more generic family of pyralomycin antibiotics.

In one embodiment of the invention Saccharic acid can be used as detergent to substitute for phosphates, as corrosion inhibitor and/or as a building block in the synthesis of polymers, such as nylon.

[0450.1.7.17] to [0452.1.7.17] for the disclosure of these paragraphs see [0450.1.7.7] to [0452.1.7.7] above

Further in another embodiment, the present invention relates to the use of the nucleic acid molecule of the invention or used in the method of the invention alone or in combination with other genes of the respective fine chemical synthesis for example of the saccharide (carbohydrates) biosynthesis, the polypeptide of the invention or used in the method of the invention, the nucleic acid construct of the invention, the expression cassette of the invention, the vector of the invention, the plant or plant tissue or the host cell of the invention, for the production of plant resistant to a herbicide inhibiting eventually the production of the fine chemical.

for the disclosure of this paragraph see [0454.1.7.7] above

In one embodiment, the present invention relates to a method for the identification of a gene product conferring the production of or an increase in the fine chemical production in a non-human cell, comprising the following steps:
(a) contacting, e.g. hybridizing, a, some or all nucleic acid molecules of a sample, e.g. cells, tissues, plants or microorganisms or a nucleic acid library, which can contain a candidate gene encoding a gene product conferring the production of or an increase in the fine chemical after expression, with the nucleic acid molecule of the present invention as shown in the respective line in column 5 or 8 of Table I A or B of application no. 17, preferably the coding region thereof, or a fragment or homolog thereof;
(b) identifying the nucleic acid molecules, which hybridize under relaxed stringent conditions with the nucleic acid molecule of the present invention in particular to the nucleic acid molecule sequence shown in the respective line in Table I, application no. 17, columns 5 or 8, preferably the coding region thereof, of a fragment or a homolog thereof, preferably in Table I B, application no. 17, columns 5 or 8, preferably the coding region thereof, of a fragment or a homolog thereof, and, optionally, isolating the full length cDNA clone or complete genomic clone;
(c) introducing the candidate nucleic acid molecules or a fragment thereof in host cells, preferably in a plant cell or a microorganism, appropriate for producing the fine chemical;
(d) expressing the identified nucleic acid molecules in the host cells for which the production of or the increased production of the fine chemical is desired;
(e) assaying the fine chemical level in the non-human host cells; and
(f) identifying the nucleic acid molecule and/or its gene product which expression confers the production of or an increase in the fine chemical level in the non-human host cell after expression compared to the wild type.

for the disclosure of this paragraph see [0456.1.7.7] above

In another embodiment, the present invention relates to a method for the identification of a gene product conferring the production of or an increase in the fine chemical production in a cell, comprising the following steps:
(a) identifying a nucleic acid molecule of a non-human organism, which is at least 20%, 25%, 30%, 35%, 40%, 50%, 60%, 70%, 80%, 90%, 95%, 96%, 97%, 98%, 99%, 99.5% or more homolog to the nucleic acid molecule encoding a polypeptide comprising the polypeptide molecule as shown in the respective line in column 5 or 8 of Table II, application no. 17, or comprising a consensus sequence or a polypeptide motif as shown in the respective line in column 8 of Table IV, application no. 17, or being encoded by a nucleic acid molecule comprising a polynucleotide as shown in the respective line in column 5 or 8 of Table I, application no. 17, preferably the coding region thereof, or a homologue thereof as described herein, for example via homology search in a data bank;
(b) introducing the candidate nucleic acid molecule in non-human host cells, preferably in a plant cell or a microorganism, appropriate for producing the fine chemical;
(c) expressing the identified nucleic acid molecule in the non-human host cells;
(d) assaying the fine chemical level in the non-human host cells; and
(e) identifying the nucleic acid molecule and its gene product which expression confers the production of or an increase in the fine chemical level in the non-human host cell after expression compared to the wild type.

for the disclosure of this paragraph see [0458.1.7.7] above

Further, the nucleic acid molecules disclosed herein, in particular the nucleic acid molecules shown in the respective line in column 5 or 8 of Table I A or B of application no. 17, may be sufficiently homologous to the sequences of related species such that these nucleic acid molecules may serve as markers for the construction of a genomic map in related organism or for association mapping. Furthermore natural variation in the genomic regions corresponding to nucleic acids disclosed herein, in particular the nucleic acid molecule shown in the respective line in column 5 or 8 of Table I A or B, of application no. 17 or homologous thereof, may lead to variation in the activity of the proteins disclosed herein, in particular the proteins comprising polypeptides as shown in the respective lines in column 5 or 8 of Table II A or B, of application no. 17 or their homologous, or comprising the consensus sequence or the polypeptide motif as shown in the respective line in column 8 of Table IV, and in consequence in a natural variation of an increased fine chemical production.

In consequence natural variation eventually also exists in form of more active allelic variants leading already to a relative increase in the fine chemical. Different variants of the nucleic acid molecules disclosed herein, in particular the nucleic acid molecules comprising the nucleic acid molecules as shown in the respective line in column 5 or 8 of Table I A or B of application no. 17, preferably the coding region thereof, which corresponds to different levels of increase in fine chemical, e.g. different levels of increase in fine chemical, can be indentified and used for marker assisted breeding for an enhanced production of the fine chemical.

Natural variation may also exist in the regulatory regions, e.g. the promoters of the natural genes comprising the nucleic acid molecules as shown in the respective line column 5 or 8 of Table I A or B of application no. 17, preferably the coding region thereof, which can similarly correspond to different levels of increase in the fine chemical, e.g. different levels of increase in the fine chemical can be identified and used for marker assisted breeding for an enhanced production of the fine chemical.

Accordingly, the present invention relates to a method for breeding plants for the production of the fine chemical, comprising
(a) selecting a first plant variety with an increased production of the fine chemical, e.g. based on an increased expression of a nucleic acid molecule of the invention as disclosed herein, in particular of a nucleic acid molecule comprising a nucleic acid molecule as shown in the respective line in column 5 or 8 of Table I A or B of application no. 17, preferably the coding region thereof, or fragments or homologs thereof, or a polypeptide comprising a polypeptide as shown in the respective line in column 5 or 8 of Table II A or B of application no. 17, or fragments or homologs thereof, or comprising a consensus sequence or a polypeptide motif as shown in the respective line in column 8 of Table IV, or a homolog thereof, as described herein;
(b) associating the level of increased production of the fine chemical, with the expression level or the genomic structure of a gene encoding said polypeptide or said nucleic acid molecule;
(c) crossing the first plant variety with a second plant variety, which significantly differs in its level of fine chemical; and
(d) identifying, which of the offspring varieties has got increased production of the fine chemical by the expression level of said polypeptide or nucleic acid molecule or the genomic structure of the gene(s) encoding said polypeptide or nucleic acid molecule of the invention.

[0462.1.7.17] to [0479.1.7.17] for the disclosure of these paragraphs see [0462.1.7.7] to [0479.1.7.7] above

In a further embodiment the nucleic acid molecule of the invention, the polypeptide of the invention, the nucleic acid construct of the invention, the expression cassette of the invention, the non-human organisms, the non-human host cell, the microorganisms, the plant, plant cell, or the part thereof, of the invention, the vector of the invention, the agonist identified with the method of the invention, the nucleic acid molecule identified with the method of the present invention, can be used for the production of the fine chemical saccharides or of the fine chemical sacharides and one or more other saccharides (carbohydrates).

Accordingly, the nucleic acid molecule of the invention, or the nucleic acid molecule identified with the method of the present invention or the complement sequences thereof, the polypeptide of the invention, the nucleic acid construct of the invention, the expression cassette of the invention, the non-human organism, the non-human host cell, the microorganism, the plant, plant cell, or the part thereof of the invention, the vector of the invention, the agonist identified with the method of the invention, the antibody of the present invention, can be used for the production of or the increase in the fine chemical in a non-human organism or part thereof, e.g. in a cell.

for the disclosure of this paragraph see [0481.1.7.7] above

for the disclosure of this paragraph see [0482.1.7.7] above

A special embodiment of this invention is related to genes which increase or generate the production of the fine chemical fructose, glucose, raffinose and/or trehalose in plant cells, plants or part thereof. Phenotypes thereto are associated with yield of plants (=yield related phenotypes). In accordance with the invention, therefore, the respective genes identified in Table I, wherein in column 7 fructose, glucose, raffinose and/or trehalose are mentioned, especially the coding region thereof, or homologs or fragments thereof,may be employed to enhance any yield-related phenotype. Increased yield may be determined in field trials of transgenic plants and suitable control plants. Alternatively, a transgene's ability to increase yield may be determined in a model plant. An increased yield phenotype may be determined in the field test or in a model plant by measuring any one or any combination of the following phenotypes, in comparison to a control plant: yield of dry harvestable parts of the plant, yield of dry aerial harvestable parts of the plant, yield of underground dry harvestable parts of the plant, yield of fresh weight harvestable parts of the plant, yield of aerial fresh weight harvestable parts of the plant yield of underground fresh weight harvestable parts of the plant, yield of the plant's fruit (both fresh and dried), grain dry weight, yield of seeds (both fresh and dry), and the like. The most basic yield-related phenotype is increased yield associated with the presence of the gene or a homolog or a fragment thereof as a transgene in the plant, i.e., the intrinsic yield of the plant. Intrinsic yield capacity of a plant can be, for example, manifested in a field test or in a model system by demonstrating an improvement of seed yield (e.g. in terms of increased seed/grain size, increased ear number, increased seed number per ear, improvement of seed filling, improvement of seed composition, embryo and/or endosperm improvements, and the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under non-stressed conditions), improvement in plant architecture.

Increased yield-related phenotypes may also be measured to determine tolerance to abiotic environmental stress. Abiotic stresses include drought, low temperature, salinity, osmotic stress, shade, high plant density, mechanical stresses, and oxidative stress, and yield-related phenotypes are encompassed by tolerance to such abiotic stresses. Additional phenotypes that can be monitored to determine enhanced tolerance to abiotic environmental stress include, without limitation, wilting; leaf browning; loss of turgor, which results in drooping of leaves or needles stems, and flowers; drooping and/or shedding of leaves or needles; the leaves are green but leaf angled slightly toward the ground compared with controls; leaf blades begun to fold (curl) inward; premature senescence of leaves or needles; loss of chlorophyll in leaves or needles and/or yellowing. Any of the yield-related phenotypes described above may be monitored in field tests or in model plants to demonstrate that a transgenic plant has increased tolerance to abiotic environmental stress. In accordance with the invention, the respective genes identified in Table 1, in case in column 7 fructose, glucose, raffinose and/or trehalose is indicated, especially the coding region thereof, or homologs or fragments thereof, may be employed to enhance tolerance to abiotic environmental stress in a plant means that the plant, when confronted with abiotic environmental stress.

A polypeptide conferring an yield-increasing activity can be encoded by a respective nucleic acid sequence as shown in Table I, column 5 or 8, and/or comprises or consists of a respective polypeptide as depicted in Table II, column 5 and 8, and/or can be amplified with the respective primer set shown in Table III, column 8, in case in column 7 fructose, glucose, raffinose and/or trehalose is indicated.

“Improved adaptation” to environmental stress like e.g. freezing and/or chilling temperatures refers to an improved plant performance under environmental stress conditions.

A modification, i.e. an increase, can be caused by endogenous or exogenous factors. For example, an increase in activity in an organism or a part thereof can be caused by adding a gene product or a precursor or an activator or an agonist to the media or nutrition or can be caused by introducing said subjects into an organism, transient or stable. Furthermore such an increase can be reached by the introduction of the respective inventive nucleic acid sequence or the encoded protein in the correct cell compartment for example into the nucleus or cytoplasmic respectively or into plastids either by transformation and/or targeting.

The term “yield” as used herein generally refers to a measurable produce from a plant, particularly a crop. Yield and yield increase (in comparison to a non-transformed starting or wild-type plant) can be measured in a number of ways, and it is understood that a skilled person will be able to apply the correct meaning in view of the particular embodiments, the particular crop concerned and the specific purpose or application concerned. The terms “improved yield” or “increased yield” can be used interchangeable.

As used herein, the term “improved yield” or the term “increased yield” means any improvement in the yield of any measured plant product, such as grain, fruit or fiber. In accordance with the invention, changes in different phenotypic traits may improve yield. For example, and without limitation, parameters such as floral organ development, root initiation, root biomass, seed number, seed weight, harvest index, tolerance to abiotic environmental stress, leaf formation, phototropism, apical dominance, and fruit development, are suitable measurements of improved yield. Any increase in yield is an improved yield in accordance with the invention. For example, the improvement in yield can comprise a 0.1%, 0.5%, 1%, 3%, 5%, 10%, 15%, 20%, 30%, 40%, 50%, 60%, 70%, 80%, 90% or greater increase in any measured parameter. For example, an increase in the bushel/acre yield of soybeans or corn derived from a crop comprising plants which are transgenic for the nucleotides and polypeptides of Table I, in case in column 7 fructose, glucose, raffinose and/or trehalose is mentioned, as compared with the bushel/acre yield from untreated soybeans or corn cultivated under the same conditions, is an improved yield in accordance with the invention. The increased or improved yield can be achieved in the absence or presence of stress conditions.

For example, enhanced or increased “yield” refers to one or more yield parameters selected from the group consisting of biomass yield, dry biomass yield, aerial dry biomass yield, underground dry biomass yield, fresh-weight biomass yield, aerial fresh-weight biomass yield, underground fresh-weight biomass yield; enhanced yield of harvestable parts, either dry or fresh-weight or both, either aerial or underground or both; enhanced yield of crop fruit, either dry or fresh-weight or both, either aerial or underground or both; and preferably enhanced yield of seeds, either dry or fresh-weight or both, either aerial or underground or both. Crop yield is defined herein as the number of bushels of relevant agricultural product (such as grain, forage, or seed) harvested per acre. Crop yield is impacted by abiotic stresses, such as drought, heat, salinity, and cold stress, and by the size (biomass) of the plant. Traditional plant breeding strategies are relatively slow and have in general not been successful in conferring increased tolerance to abiotic stresses. Grain yield improvements by conventional breeding have nearly reached a plateau in maize.

Accordingly, the yield of a plant can depend on the specific plant/crop of interest as well as its intended application (such as food production, feed production, processed food production, biofuel, biogas or alcohol production, or the like) of interest in each particular case. Thus, in one embodiment, yield is calculated as harvest index (expressed as a ratio of the weight of the respective harvestable parts divided by the total biomass), harvestable parts weight per area (acre, square meter, or the like); and the like. The harvest index, i.e., the ratio of yield biomass to the total cumulative biomass at harvest, in maize has remained essentially unchanged during selective breeding for grain yield over the last hundred years. Accordingly, recent yield improvements that have occurred in maize are the result of the increased total biomass production per unit land area. This increased total biomass has been achieved by increasing planting density, which has led to adaptive phenotypic alterations, such as a reduction in leaf angle, which may reduce shading of lower leaves, and tassel size, which may increase harvest index. Harvest index is relatively stable under many environmental conditions, and so a robust correlation between plant size and grain yield is possible. Plant size and grain yield are intrinsically linked, because the majority of grain biomass is dependent on current or stored photosynthetic productivity by the leaves and stem of the plant. As with abiotic stress tolerance, measurements of plant size in early development, under standardized conditions in a growth chamber or greenhouse, are standard practices to measure potential yield advantages conferred by the presence of a transgene.

Acccordingly, the yield of a plant can be increased by improving one or more of the yield-related phenotypes.

Such yield-related phenotypes or traits of a plant the improvement of which results in increased yield comprise, without limitation, the increase of the intrinsic yield capacity of a plant, improved nutrient use efficiency, and/or increased stress tolerance.

For example, yield refers to biomass yield, e.g. to dry weight biomass yield and/or fresh-weight biomass yield. Biomass yield refers to the aerial or underground parts of a plant, depending on the specific circumstances (test conditions, specific crop of interest, application of interest, and the like). In one embodiment, biomass yield refers to the aerial and underground parts. Biomass yield may be calculated as fresh-weight, dry weight or a moisture adjusted basis. Biomass yield may be calculated on a per plant basis or in relation to a specific area (e.g. biomass yield per acre/square meter/ or the like).

In other embodiment, “yield” refers to seed yield which can be measured by one or more of the following parameters: number of seeds or number of filled seeds (per plant or per area (acre/square meter/ or the like)); seed filling rate (ratio between number of filled seeds and total number of seeds); number of flowers per plant; seed biomass or total seeds weight (per plant or per area (acre/square meter/ or the like); thousand kernel weight (TKW; extrapolated from the number of filled seeds counted and their total weight; an increase in TKW may be caused by an increased seed size, an increased seed weight, an increased embryo size, and/or an increased endosperm). Other parameters allowing to measure seed yield are also known in the art. Seed yield may be determined on a dry weight or on a fresh weight basis, or typically on a moisture adjusted basis, e.g. at 15.5 percent moisture.

For example, the term “increased yield” means that the plant, exhibits an increased growth rate, under conditions of abiotic environmental stress, compared to the corresponding wild-type plant.

An increased growth rate may be reflected inter alia by or confers an increased biomass production of the whole plant, or an increased biomass production of the aerial parts of a plant, or by an increased biomass production of the underground parts of a plant, or by an increased biomass production of parts of a plant, like stems, leaves, blossoms, fruits, and/or seeds.

Increased yield includes higher fruit yields, higher seed yields, higher fresh matter production, and/or higher dry matter production.

The term “increased yield” means that the plant, exhibits a prolonged growth under conditions of abiotic environmental stress, as compared to the corresponding, e.g. non-transformed, wild type organism. A prolonged growth comprises survival and/or continued growth of the plant, at the moment when the non-transformed wild type organism shows visual symptoms of deficiency and/or death.

When the plant of the invention is a corn plant increased yield for corn plants means, for example, increased seed yield, in particular for corn varieties used for feed or food. Increased seed yield of corn refers to an increased kernel size or weight, an increased kernel per ear, or increased ears per plant. Alternatively or in addition the cob yield may be increased, or the length or size of the cob is increased, or the kernel per cob ratio is improved.

When the plant of the invention is a soy plant, increased yield for soy plants means increased seed yield, in particular for soy varieties used for feed or food. Increased seed yield of soy refers for example to an increased kernel size or weight, an increased kernel per pod, or increased pods per plant.

When the plant of the invention is an oil seed rape (OSR) plant, increased yield for OSR plants means increased seed yield, in particular for OSR varieties used for feed or food. Increased seed yield of OSR refers to an increased seed size or weight, an increased seed number per silique, or increased siliques per plant.

When the plant of the invention is a cotton plant, increased yield for cotton plants means increased lint yield. Increased lint yield of cotton refers in one embodiment to an increased length of lint.

When the plant of the invention is a wheat plant increased yield for wheat plants means, for example, increased seed yield, in particular for wheat varieties used for feed or food. Increased seed yield of wheat refers to an increased kernel size or weight, an increased kernel per ear, or increased ears per plant. Increased seed yield of wheat refers also to an increased grain size or weight, an increased number of grains per spike, or increased number of spikes per plant. Alternatively or in addition increased seed yield of wheat refers also to an increased grain yield, increased length of spikes, increased number of spikelets per spike , increased 1000 grain weight or increased flag leaf area (flag leaf is the leaf directely below the spike).

Said increased yield can typically be achieved by enhancing or improving, one or more yield-related traits of the plant. Such yield-related traits of a plant comprise, without limitation, the increase of the intrinsic yield capacity of a plant, improved nutrient use efficiency, and/or increased stress tolerance, in particular increased abiotic stress tolerance.

Intrinsic yield capacity of a plant can be, for example, manifested by improving the specific (intrinsic) seed yield (e.g. in terms of increased seed/ grain size, increased ear number, increased seed number per ear, improvement of seed filling, improvement of seed composition, embryo and/or endosperm improvements, or the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under stressed or non-stressed conditions), improvement in plant architecture, cell cycle modifications, photosynthesis modifications, various signaling pathway modifications, modification of transcriptional regulation, modification of translational regulation, modification of enzyme activities, and the like); and/or the like.

The improvement or increase of stress tolerance of a plant can for example be manifested by improving or increasing a plant's tolerance against stress, particularly abiotic stress. In the present application, abiotic stress refers generally to abiotic environmental conditions a plant is typically confronted with, including, but not limited to, drought (tolerance to drought may be achieved as a result of improved water use efficiency), heat, low temperatures and cold conditions (such as freezing and chilling conditions), salinity, osmotic stress , shade, high plant density, mechanical stress, oxidative stress, and the like.

The increased plant yield can also be mediated by increasing the “nutrient use efficiency of a plant”, e.g. by improving the use efficiency of nutrients including, but not limited to, phosphorus, potassium, and nitrogen. Further, higher yields may be obtained with current or standard levels of nitrogen use.

Generally, the term “increased tolerance to stress” can be defined as survival of plants, and/or higher yield production, under stress conditions as compared to a non-transformed wild type or starting plant: For example, the plant of the invention or produced according to the method of the invention is better adapted to the stress conditions. “Improved adaptation” to environmental stress like e.g. drought, heat, nutrient depletion, freezing and/or chilling temperatures refers herein to an improved plant performance resulting in an increased yield, particularly with regard to one or more of the yield related traits as defined in more detail above.

During its life-cycle, a plant is generally confronted with a diversity of environmental conditions. Any such conditions, which may, under certain circumstances, have an impact on plant yield, are herein referred to as “stress” condition. Environmental stresses may generally be divided into biotic and abiotic (environmental) stresses. Unfavorable nutrient conditions are sometimes also referred to as “environmental stress”. The present invention does also contemplate solutions for this kind of environmental stress, e.g. referring to increased nutrient use efficiency.

For the purposes of the description of the present invention, the terms “enhanced tolerance to abiotic stress”, “enhanced resistance to abiotic environmental stress”, “enhanced tolerance to environmental stress”, “improved adaptation to environmental stress” and other variations and expressions similar in its meaning are used interchangeably and refer, without limitation, to an improvement in tolerance to one or more abiotic environmental stress(es) as described herein and as compared to a corresponding origin or wild type plant or a part thereof.

The term abiotic stress tolerance(s) refers for example low temperature tolerance, drought tolerance or improved water use efficiency (WUE), heat tolerance, salt stress tolerance and others.

Studies of a plant's response to desiccation, osmotic shock, and temperature extremes are also employed to determine the plant's tolerance or resistance to abiotic stresses. Water use efficiency (WUE) is a parameter often correlated with drought tolerance. In selecting traits for improving crops, a decrease in water use, without a change in growth would have particular merit in an irrigated agricultural system where the water input costs were high. An increase in growth without a corresponding jump in water use would have applicability to all agricultural systems. In many agricultural systems where water supply is not limiting, an increase in growth, even if it came at the expense of an increase in water use also increases yield.

Drought stress means any environmental stress which leads to a lack of water in plants or reduction of water supply to plants, including a secondary stress by low temperature and/or salt, and/or a primary stress during drought or heat, e.g. desiccation etc.

### Specific Embodiments

Accordingly, this invention provides respective measures and methods to produce plants with increased yield, e.g. genes conferring an increased yield-related trait, for example enhanced tolerance to abiotic environmental stress, for example an increased drought tolerance and/or low temperature tolerance and/or an increased nutrient use efficiency, intrinsic yield and/or another increased yield-related trait, upon expression or over-expression. Accordingly, the present invention provides such genes in case in column 7 of the respective Table or fructose, glucose, raffinose and/or trehalose is indicated. In particular, such genes are described in column 5 as well as in column 8 of Tables I, especially the coding region thereof, or homologs or fragments thereof, in case fructose, glucose, raffinose and/or trehalose are indicated in column 7 or the respective polypeptides are described in column 5 as well as in column 8 of Table II, or homologs or fragments thereof, in case fructose, glucose, raffinose and/or trehalose are indicated in column 7.

Accordingly, the present invention provides respective transgenic plants showing one or more improved yield-related traits as compared to the corresponding origin or the wild type plant and methods for producing such transgenic plants with increased yield in case in column 7 of the respective Table “fructose” or “glucose” or “raffinose” or “trehalose” is indicated.

In one embodiment, one or more of said yield-increasing activities are increased by increasing the amount and/or the specific activity of one or more proteins listed in Table II, column 5 or 8 in a compartment of a cell indicated in Table II, column 6, in case in column 7 of the respective Table “fructose” or “glucose” or “raffinose” or “trehalose” is indicated.

Accordingly to present invention, the yield of the plant of the invention is increased by improving one or more of the yield-related traits as defined herein. Said increased yield in accordance with the present invention can typically be achieved by enhancing or improving, in comparison to an origin or wild-type plant, one or more yield-related traits of said plant. Such yield-related traits of a plant the improvement of which results in increased yield comprise, without limitation, the increase of the intrinsic yield capacity of a plant, improved nutrient use efficiency, like nitrogen use efficiency, and/or increased stress tolerance.

In one embodiment, the present invention provides respective transgenic plants showing one or more improved yield-related traits, preferably increased intrinsic yield as compared to the corresponding origin or the wild type plant and methods for producing such transgenic plants with increased yield in case in column 7 of the respective Table “fructose” or “glucose” or “trehalose” is indicated.

In one embodiment, the present invention provides respective transgenic plants showing one or more improved yield-related traits, preferably increased drought and/or increased cold stress tolerance as compared to the corresponding origin or the wild type plant and methods for producing such transgenic plants with increased yield in case in column 7 of the respective Table “raffinose”.

In a further embodiment the present invention is characterized by the features as disclosed in the following items:

Item 1. A process for the production of a fine chemical selected from the group consisting of: 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, and/or verbascose
(a) increasing or generating one or more activities selected from the group consisting of 1,4-alpha-glucan branching enzyme, 2,3-dihydroxy-2,3-dihydrophenylpropionatedehydrogenase, 2-C-methyl-D-erythritol 4-phosphate cytidylyltransferase, 2-oxoglutarate dehydrogenase E1 subunit, 3-deoxy-7-phosphoheptulonate synthase, 3-isopropylmalate dehydratase large subunit, 3-phosphoglycerate dehydrogenase, 47266012-protein, 49747384_SOYBEAN-protein, 4-alpha-glucanotransferase, ABC transporter permease protein, acetyl CoA carboxylase, acetyl CoA synthetase, acetyl-coenzyme A synthetase, acetyltransferase, acid shock protein, acyl carrier protein, acyl transferase, acyl-CoA dehydrogenase, acyl-CoA synthase, adenylosuccinate lyase, alanine dehydrogenase, alcohol dehydrogenase, aldolase, alkaline phosphatase, amine oxidase, amino-acid acetyltransferase, aminotransferase, arginine exporter protein, asparagine synthetase B, At1g19350-protein, At2g45420-protein, At5g16650-protein, At5g43630-protein, ATP synthase subunit E, ATP-binding component of a transport system, auxin response factor, AX653549-protein, AY087308-protein, b0050-protein, b0251-protein, b0362-protein, b0488-protein, B0644-protein, b0801-protein, b0970-protein, b1003-protein, b1046-protein, b1137-protein, b1214-protein, b1219-protein, b1234-protein, b1285-protein, b1357-protein, b1410-protein, b1425-protein, b1445-protein, b1470-protein, b1522-protein, b1556-protein, b1583-protein, b1670-protein, b1796-protein, b1898-protein, b2032-protein, b2107-protein, b2139-protein, b2345-protein, b2360-protein, b2361-protein, b2399-protein, b2466-protein, b2474-protein, b2613-protein, b2653-protein, b2654-protein, b2670-protein, b2673-protein, b2682-protein, b2739-protein, b2932-protein, b3151-protein, b3221-protein, b3246-protein, b3346-protein, B3427-protein, b3442-protein, B3443-protein, b3777-protein, b3817-protein, b3818-protein, b3989-protein, b4029-protein, b4050-protein, b4056-protein, b4250-protein, beta-galactosidase, beta-hydroxylase, betaine aldehyde dehydrogenase, bifunctional protein (phosphoribosyltransferase and regulatory protein), binding-protein-dependent transport systems inner membrane component, branched-chain amino acid ABC transporter permease protein, branched-chain amino acid permease, calcium-dependent protein kinase, carbamoyl-phosphate synthase subunit, cardiolipin synthase, cation-transporting ATPase, CBL-interacting protein kinase, CCAAT-binding transcription factor, CDP-diglyceride synthetase, cell division control protein, cell division protein, chlorophyllase, chorismate lyase, circadian clock protein, colanic acid biosynthesis protein, coproporphyrinogen III oxidase, coproporphyrinogen oxidase, CTP synthase, cullin, cyclin, cyclin D, cystathionine gamma-synthase, cysteine synthase A, dATP pyrophosphohydrolase, dihydrolipoamide dehydrogenase, DNA binding-protein, DNA replication complex GINS protein, DnaJ-like chaperone, electron transfer flavoprotein subunit beta, electron transport complex protein, elongation factor Tu, enoyl-CoA hydratase, eukaryotic translation initiation factor, eukaryotic translation initiation factor 5, fatty acid desaturase, ferredoxin, Fe—S subunit of oxidoreductase, flagellar M-ring protein, flavodoxin, fructose biphosphate aldolase, fumarylacetoacetate hydrolase, galactinol synthase, GDP-mannose 4,6-dehydratase, GDP-mannose dehydrogenase, geranylgeranyl pyrophosphate synthase, gibberellin 20-oxidase, Glu/Leu/Phe/Val dehydrogenase , glucokinase, gluconate transport system permease 3, glucose dehydrogenase, glucose-6-phosphate 1-dehydrogenase, glutamate-ammonia-ligase, glutamine amidotransferase, glutamine synthetase, glutaredoxin, glycerate kinase, glycogen (starch) synthase, glycogen synthase, glycosidase, glycosyl transferase, glycosyltransferase, GM02LC38418-protein, GM02LC46-protein, halotolerance protein, heat shock transcription factor, hexokinase, hexose transporter, high-affinity branched-chain amino acid transport protein, histone acetyltransferase, homocitrate synthase, hydrolase, inositol-3-phosphate synthase, integral membrane protein, intracellular septation protein, ion-transport protein, iron(III) dicitrate-binding protein, isochorismate synthase, isocitrate dehydrogenase, isopropylmalate isomerase large subunit, ketol-acid reductoisomerase, kinase, leucyl/phenylalanyl-tRNA-protein transferase, lipoprotein precursor, L-ribulose-5-phosphate 4-epimerase, L-serine dehydratase, lysine decarboxylase, MADS box protein transcription factor, MADS-box transcription factor, malate dehydrogenase, mannitol-1-phosphate dehydrogenase, membrane protein, membrane transport protein, methylglyoxalase, methyltransferase, Mg-protoporphyrin IX monomethyl ester oxidative cyclase 66 kD subunit, mitogen-activated protein kinase, molecular chaperone, monothiol glutaredoxin, monthiol glutaredoxin, multidrug resistance protein, multiple antibiotic resistance protein, murein transglycosylase, NAD(P)H dehydrogenase (quinone), NADH dehydrogenase, NADH-quinone oxidoreductase subunit, O-acetyltransferase, O-antigen chain length determinant, outer membrane receptor protein precursor, oxidoreductase, oxidoreductase subunit, para-aminobenzoate synthase component I, penicillin-binding protein, peptidyl-prolyl cis-trans isomerase, permease, permease protein of ABC transporter, phage-related repressor protein, phenylacetic acid degradation protein, phenylalanine ammonia-lyase, phosphate acetyltransferase, phosphate permease, phosphatidylinositol 3- and 4-kinase family protein, phosphoadenosine phosphosulfate reductase, phosphoanhydride phosphorylase, phosphoenolpyruvate carboxykinase, phosphoglycerate kinase, phosphohydrolase, phosphotransferase system component, photosystem I reaction center subunit XI, photosystem I reaction centre subunit, photosystem II protein, polyamine transporter, precorrin methylase, protease, protein kinase, protein phosphatase, protein-L-isoaspartate O-methyltransferase, purine biosynthesis protein, purine nucleoside phosphorylase, purine-nucleoside phosphorylase, putative transport system permease protein, putrescine transport system permease protein, pyridoxal phosphate biosynthetic protein, pyrimidine-specific ribonucleoside hydrolase, pyruvate kinase, recombinase A, RNA-binding protein, sec-independent protein translocase, Sec-independent protein translocase subunit, sensor histidine kinase, serine protease, serine/threonine dehydratase, serine/threonine-protein phosphatase, short chain dehydrogenase, short-chain alcohol dehydrogenase family, sll0254-protein, sll0281-protein, sll0354-protein, sll0418-protein, sll1761-protein, slr0600-protein, slr1107-protein, sn-glycerol-3-phosphate dehydrogenase, sn-glycerol-3-phosphate transport system permease protein, sodium/proton antiporter, sterol O-acyltransferase, subunit of COMPASS-protein complex, succinyl-CoA synthetase beta chain, ThiF family protein, thioredoxin, thioredoxin family protein, threonine dehydratase, threonine efflux protein, threonine synthase, transcription factor, transcriptional regulator, transcriptional regulator protein, transport protein, trehalase, trehalose-6-phosphate synthase, trehalose-phosphatase, tryptophan biosynthesis protein, TTC1386-protein, urease accessory protein, urease subunit, vacuolar sorting protein, XM_{—}473199-protein, ycl027w-protein, YCR047W-A-protein, ycr102c-protein, YDL159W-A-protein, ydl235c-protein, ydr183w-protein, ydr507c-protein, YEL030C-A-protein, yer177w-protein, YFL019C-protein, ygl096w-protein, ygl237c-protein, yhl013c-protein, YIL083C-protein, YJL127W-A-protein, ymr324c-protein, yol160w-protein, ypl144w-protein, ypr098c-protein, zinc transporter, and Zm_{—}4842_BE510522-protein, in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, and/or verbascose or a composition comprising 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, and/or verbascose in said non-human organism or in the culture medium surrounding said non-human organism.

Item 2. A process for the production of a fine chemical selected from the group consisting of: 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, and/or verbascose, which comprises
(A)
   (i) increasing or generating of the expression of; and/or
   (ii) increasing or generating the expression of an expression product of; and/or
   (iii) increasing or generating one or more activities of an expression product encoded by;
      at least one respective nucleic acid molecule comprising a nucleic acid molecule selected from the group consisting of:
   (a) a nucleic acid molecule encoding the polypeptide shown in column 5 or 8 of Table II, preferably Table II B, application no.17, or a homolog or a fragment thereof;
   (b) a nucleic acid molecule shown in column 5 or 8 of Table I, preferably Table I B, application no.17, or a homolog or a fragment thereof (preferably the coding region thereof);
   (c) a nucleic acid molecule, which, as a result of the degeneracy of the genetic code, can be derived from a polypeptide sequence depicted in column 5 or 8 of Table II, preferably Table II B, application no.17;
   (d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 17, or the coding region thereof;
   (e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no.17;
   (i) a nucleic acid molecule encoding a polypeptide having the activity represented by a respective protein as depicted in column 5 of Table II, application no. 17;
   (j) a nucleic acid molecule which comprises a polynucleotide, which is obtained by amplifying a cDNA library or a genomic library using the primers as depicted in column 8 of Table III, application no. 17; and
   (k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
   or a nucleic acid molecule comprising a sequence which is complementary thereto;
      in a non-human organism or a part thereof, preferably a microorganism, a plant cell, a plant or a part thereof, as compared to a corresponding non-transformed wild type non-human organism or a part thereof; and
(B) growing the non-human organism or a part thereof under conditions which permit the production of 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, and/or verbascose or a composition comprising 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, and/or verbascose in said non-human organism or in the culture medium surrounding said non-human organism.

Item 3. A process of items 1 or 2, comprising of recovering 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, and/or verbascose in its free or bound form.

Item 4. The process of any one of items 1 to 3, comprising the following steps:
(a) selecting a non-human organism or a part thereof expressing a polypeptide encoded by the nucleic acid molecule characterized in item 2;
(b) mutagenizing the selected non-human organism or the part thereof;
(c) comparing the activity or the expression level of said polypeptide in the mutagenized non-human organism or the part thereof with the activity or the expression of said polypeptide of the selected non-human organisms or the part thereof;
(d) selecting the mutated non-human organisms or parts thereof, which comprise an increased activity or expression level of said polypeptide compared to the selected non-human organism or the part thereof;
(e) optionally, growing and cultivating the non-human organisms or the parts thereof; and
(f) recovering, and optionally isolating, the free or bound 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, and/or verbascose produced by the selected mutated non-human organisms or parts thereof.

Item 5. The process of any one of items 1 to 4 wherein the activity of said protein or the expression of said nucleic acid molecule is increased or generated transiently or stably.

Item 6. An isolated nucleic acid molecule comprising a nucleic acid molecule selected from the group consisting of:
(a) a nucleic acid molecule encoding a polypeptide shown in Table II, application no. 17, column 5 or 8, preferably shown in Table II B, application no. 17, column 8, or a homolog or a fragment thereof;
(b) a nucleic acid molecule shown in Table I, application no. 17, column 5 or 8, preferably shown in Table I B, application no. 17, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 17, preferably in column 8 of Table II B, application no. 17;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 17, preferably shown in column 8 of Table I B, application no. 17, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 17;
(i) a nucleic acid molecule encoding a polypeptide having the activity represented by a respective protein as depicted in column 5 of Table II, application no. 17;
(j) a nucleic acid molecule comprising a polynucleotide which is obtained by amplifying a cDNA library or a genomic library using the primers in Table III, application no. 17, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j); or a nucleic acid molecule comprising a sequence which is complementary thereto.

Item 7. A nucleic acid construct which confers the expression of the nucleic acid molecule as defined in item 6, comprising one or more regulatory elements.

Item 8. An expression cassette comprising
(a) a promoter, preferably selected from the group consisting of Big35S, PCUbi, Super and USP;
(b) a nucleic acid molecule as defined in item 6.

Item 9. A vector comprising the nucleic acid molecule as defined in item 6 or the nucleic acid construct as claimed in item 7 or the expression cassette as claimed in item 8.

Item 10. A host cell or a plant organelle, which has been transformed with the vector as claimed in item 9 or the nucleic acid molecule as defined in item 6 or the nucleic acid construct as claimed in item 7 or the expression cassette as claimed in item 8.

Item 11. The host cell of item 10, which is a plant cell or a microorganism.

Item 12. A process for producing a polypeptide, wherein the polypeptide is expressed in a host cell as claimed in item 10 or 11.

Item 13. A polypeptide produced by the process as claimed in item 12 or encoded by the nucleic acid molecule as defined in item 6.

Item 14. An antibody, which binds specifically to the polypeptide as claimed in item 13.

Item 15. A plant tissue, propagation material, harvested material or a plant comprising the host cell as claimed in item 10 or 11.

Item 16. A process for the identification of a compound conferring an increase in 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, and/or verbascose production in a non-human organism, comprising the steps:
(a) culturing a plant cell or tissue or microorganism or maintaining a plant expressing the polypeptide encoded by the nucleic acid molecule as defined in item 6 conferring an increase in the amount of 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, and/or verbascose in a non-human organism or a part thereof and a readout system capable of interacting with the polypeptide under suitable conditions which permit the interaction of the polypeptide with said readout system in the presence of a compound or a sample comprising a plurality of compounds and capable of providing a detectable signal in response to the binding of a compound to said polypeptide under conditions which permit the expression of said readout system and of the polypeptide encoded by the nucleic acid molecule of item 6 conferring an increase in the amount of 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, and/or verbascose in a non-human organism or a part thereof;
(b) identifying if the compound is an effective agonist by detecting the presence or absence or increase of a signal produced by said readout system.

Item 17. A method for the production of an agricultural composition comprising the steps of the method of item 16 and formulating the compound identified in item 16 in a form acceptable for an application in agriculture.

Item 18. A composition comprising the nucleic acid molecule as defined in item 6, the polypeptide as claimed in item 13, the nucleic acid construct as claimed in item 7, the expression cassette as claimed in item 8, the vector as claimed in item 9, the compound as claimed in item 16, the antibody as claimed in item 14, and optionally an agricultural acceptable carrier.

Item 19. Use of the nucleic acid molecule as defined in item 6 for the identification of a nucleic acid molecule conferring an increase in 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, and/or verbascose after expression.

Item 20. Cosmetic, pharmaceutical, food or feed composition comprising the nucleic acid moleculeas defined in item 6, the polypeptide as claimed in item 13, the nucleic acid construct as claimed in item 7, the expression cassette as claimed in item 8, the vector as claimed in item 9, the antibody as claimed in item 14 , the plant or plant tissue as claimed in item 15, the harvested material as claimed in item 15 or the host cell of item as claimed in items 10 or 11.

Item 21. Use of the nuclei acid molecule as defined in item 6, the polypeptide as claimed in item 13, the nucleic acid construct as claimed in item 7, the expression cassette as claimed in item 8, the vector as claimed in item 9, the plant or plant tissue as claimed in item 15, or the host cell of item 10 to 11 for the production of plant resistant to a herbicide inhibiting the production of 3,4,5-trihydroxypentanoic acid, erythrol, fructose, fucose, galactinol, galactitol, glucose, glucose-1-phosphate, mannitol, melibiose, myo-inositol, raffinose, saccharic acid, sedoheptulose-7-phosphate, sorbitol, sucrose, trehalose, and/or verbascose.

Item 22. A method for producing a plant with increased yield as compared to a corresponding non-transformed wild type plant, whereby the method comprises
(i) increasing or generating of the expression of; and/or
(ii) increasing or generating the expression of an expression product of; and/or
(iii) increasing or generating one or more activities of an expression product encoded by;
   at least one respective nucleic acid molecule comprising a nucleic acid molecule selected from the group consisting of:
(a) a nucleic acid molecule encoding the polypeptide shown in column 5 or 8 of Table II, preferably Table II B, application no. 17, or a homolog or a fragment thereof, in case in column fructose, glucose, raffinose and/or trehalose is indicated;
(b) a nucleic acid molecule shown in column 5 or 8 of Table I, preferably Table I B, application no. 17, or a homolog or a fragment thereof (preferably the coding region thereof) , in case in column fructose, glucose, raffinose and/or trehalose is indicated;
(c) a nucleic acid molecule, which, as a result of the degeneracy of the genetic code, can be derived from a polypeptide sequence depicted in column 5 or 8 of Table II, preferably Table II B, application no. 17, in case in column fructose, glucose, raffinose and/or trehalose is indicated;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 17, or the coding region thereof, in case in column fructose, glucose, raffinose and/or trehalose is indicated;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 17, in case in column fructose, glucose, raffinose and/or trehalose is indicated;
(i) a nucleic acid molecule encoding a polypeptide having the activity represented by a respective protein as depicted in column 5 of Table II, application no. 17, in case in column fructose, glucose, raffinose and/or trehalose is indicated;
(j) a nucleic acid molecule which comprises a polynucleotide, which is obtained by amplifying a cDNA library or a genomic library using the primers as depicted in column 8 of Table III, application no. 17, in case in column fructose, glucose, raffinose and/or trehalose is indicated; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto;
   in a plant cell, plant or a part thereof.

Item 23. A method for producing a plant with increased yield according to item 22, which comprises in addition the regeneration of a plant from such transformed plant cell, plant cell compartment, plant cell nucleus, plant cell or plant tissue with increased yield.

[0483.1.7.17] to [0492.1.7.17] for the disclosure of these paragraphs see [0483.1.7.7] to [0492.1.7.7] above

### Example 10

Purification of a fine chemical e.g. saccharides meaning all of the aforementioned carbohydrate, e.g. monosaccharides, preferably fructose and/or glucose, or other pentoses or hexoses; in free or bound form, preferably glucose1-phosphate and/or sedoheptulose-7-phosphate; and/or derivates of monosaccharides, preferably (sugar) acids, e.g. saccharic acid, 3,4,5-trihydroxypentanoic acid; and/or sugar alcohols e.g. erythrol, galactitol, sorbitol, mannitol and/or myo-inositol; and/or deoxy-derivates, e.g. fucose; and/or disaccharides, preferably sucrose, melibiose, galactinol and/or trehalose, and/or oligosaccharides, preferably the RFOs (raffinose family of oligosaccharides: preferably the trisaccharide raffinose and/or the pentasaccharide verbascose), and/or polysaccharides, preferably starch and/or cellulose

Saccharides (carbohydrates) can for example be detected advantageously via traditional methods of sugar analysis coupled to chromatography use a Refractive Index Detector (RID) due to a lack of a UV-absorbing chromophore on sugar molecules. Other detectors, like Mass Spectrometry (MS) or Pulsed Amperometric Detection (PAD), are used also. Methods of sugar analysis are capillary electrophoresis, GC, HPLC or LC.

Saccharides (carbohydrates) are detected by GC or LC combined with MS. Traditional methods of sugar analysis coupled to chromatography use a Refractive Index Detector (RID) due to a lack of a UV-absorbing chromophore on sugar molecules. Other detectors, like Mass Spectrometry (MS) or Pulsed Amperometric Detection (PAD), are used also.

In one embodiment of the invention the fructose can be detected by chromatography, thin layer chromatography, Gaschromatography (GC), liquid-chromatographie (LC), capillary electrophoresis and HPLC. Alternatively fructose can be detected and analized by bio-sensors: a amperometric enzyme electrode for fructose analysis was constructed, by co-immobilization of a pyrrolo quinoline quinone (PQQ) enzyme (Gluconobacter sp. fructose-5-dehydrogenase, FDH, EC-1.1.99.11) with a mediator in a thin polypyrrole (PP) membrane (Anal.Chim.Acta; (1993) 281, 3, 527-33). Two amperometric biosensors for fructose detection were developed by immobilizing d-fructose 5-dehydrogenase by two different immobilization processes (Analytica Chimica Acta, Volume 374, Number 2, 23 November 1998, pp. 201-208(8)). In one embodiment of the invention the glucose can be detected by Fourier transformed nearinfrared (FT-NIR) spectroscopy in diffuse reflectance mode (Liu et al., 2006), by HPLC (siehe z. B. Sánchez-Mata et al., European Food Research and Technology, 2004) or by colourimetric enzyme-assays (Ciantar et al., J Periodontal Res., 2002).

A further method is the analysis of fluorophore-labeled glycans by high-resolution polyacrylatmide gel electrophoresis (Jacksonet al., Anal. Biochem. 216 (1994) 243-52).

The sucrose of the invention is detected in one embodiment by traditional methods of sugar analysis coupled to chromatography use a Refractive Index Detector (RID, Koimur et al., Chromatographia 43, 1996, p. 254-260; Callul et al., J. Chromatogr. 590, 1992, p. 215-222.);) due to a lack of a UV-absorbing chromophore on sugar molecules. Other detectors, like Mass Spectrometry (MS) or Pulsed Amperometric Detection (PAD, Weston et al., Food Chem. 64, 1999, p. 33-37; Sigvardson et al., J. Pharm. Biomed. Anal. 15, 1996, p. 227-231) are also used.

In another embodiment the sucrose is detected by enzyme-linked immunosorbant assay (U.S. Pat. No. 5,9726,31), or by Fourier Transform Infrared Detection in Miniaturized Total Analysis Systems for Sucrose Analysis (Anal. Chem. 1997, 69, 2877-2881).

for the disclosure of this paragraph see [0494.1.7.7] above

### Example 11a

### PCR Amplification of the Sequences

Unless otherwise specified, standard methods as described in Sambrook et al., Molecular Cloning:

A laboratory manual, Cold Spring Harbor 1989, Cold Spring Harbor Laboratory Press are used.

The inventive sequences as shown in the respective line in Table I, column 5, preferably the coding region thereof, were amplified by PCR as described in the protocol of the Pfu Ultra, Pfu Turbo or Herculase DNA polymerase (Stratagene). The composition for the protocol of the Pfu Ultra, Pfu Turbo or Herculase DNA polymerase was as follows: 1×PCR buffer (Stratagene), 0.2 mM of each dNTP, 100 ng genomic DNA of Saccharomyces cerevisiae (strain S288C; Research Genetics, Inc., now Invitrogen), Escherichia coli (strain MG1655; E. coli Genetic Stock

Center), Synechocystis sp. (strain PCC6803), Azotobacter vinelandii (strain N.R. Smith,16), Thermus thermophilus (HB8) or 50 ng cDNA from various tissues and development stages of Arabidopsis thaliana (ecotype Columbia), Physcomitrella patens, Glycine max (variety Resnick), Brassica napus, Oryza sativa or Zea mays (variety B73, Mo17, A188), 50 pmol forward primer, 50 pmol reverse primer, with or without 1 M Betaine, 2.5 u Pfu Ultra, Pfu Turbo or Herculase DNA polymerase.

The amplification cycles were as follows:

1 cycle of 2-3 minutes at 94-95° C., then 25-36 cycles with 30-60 seconds at 94-95° C., 30-45 seconds at 50-60° C. and 210-480 seconds at 72° C., followed by 1 cycle of 5-10 minutes at 72° C., then 4-16° C.—preferably for Saccharomyces cerevisiae, Escherichia coli, Synechocystis sp., Azotobacter vinelandii, Thermus thermophilus.

In case of Arabidopsis thaliana, Brassica napus, Glycine max, Oryza sativa, Physcomitrella patens, Zea mays the amplification cycles were as follows: 1 cycle with 30 seconds at 94° C., 30 seconds at 61° C., 15 minutes at 72° C., then 2 cycles with 30 seconds at 94° C., 30 seconds at 60° C., 15 minutes at 72° C., then 3 cycles with 30 seconds at 94° C., 30 seconds at 59° C., 15 minutes at 72° C., then 4 cycles with 30 seconds at 94° C., 30 seconds at 58° C., 15 minutes at 72° C., then 25 cycles with 30 seconds at 94° C., 30 seconds at 57° C., 15 minutes at 72° C., then 1 cycle with 10 minutes at 72° C., then finally 4-16° C.

RNAs were generated with the RNeasy Plant Kit according to the standard protocol (Qiagen) and Superscript II Reverse Transkriptase was used to produce double stranded cDNA according to the standard protocol (Invitrogen).

ORF specific primer pairs for the genes to be expressed are shown in the respective line in Table III, column 8. The following adapter sequences were added to Saccharomyces cerevisiae ORF specific primers (see Table III) for cloning purposes:

SEQ ID NO: 20

| | | |
|---|---|---|
| i) | foward primer: | 5′-GGAATTCCAGCTGACCACC-3′ |

SEQ ID NO: 21

| | | |
|---|---|---|
| ii) | reverse primer: | 5′-GATCCCCGGGAATTGCCATG-3′ |

These adaptor sequences allow cloning of the ORF into the various vectors containing the Resgen adaptors, see table column E of Table c.

The following adapter sequences were added to Saccharomyces cerevisiae, Escherichia coli, Synechocystis sp., Azotobacter vinelandii, Thermus thermophilus, Arabidopsis thaliana, Brassica napus, Glycine max, Oryza sativa , Physcomitrella patens, or Zea mays ORF specific primers for cloning purposes:

| SEQ ID NO: 22 | | |
|---|---|---|
| iii) | forward primer: | 5′-TTGCTCTTCC-3′ |
| SEQ ID NO: 23 | | |
| iiii) | reverse primer: | 5′-TTGCTCTTCG-3′ |

The adaptor sequences allow cloning of the ORF into the various vectors containing the Colic adaptors, see table column E of Table c.

Therefore for amplification and cloning of Saccharomyces cerevisiae SEQ ID NO: 14171, a primer consisting of the adaptor sequence i) and the ORF specific sequence SEQ ID NO: 14271 and a second primer consisting of the adaptor sequence ii) and the ORF specific sequence SEQ ID NO: 14272 were used.

For amplification and cloning of Escherichia coli SEQ ID NO: 7081, a primer consisting of the adaptor sequence iii) and the ORF specific sequence SEQ ID NO: 7261 and a second primer consisting of the adaptor sequence iiii) and the ORF specific sequence SEQ ID NO: 7262 were used.

For amplification and cloning of Synechocystis sp. SEQ ID NO: 11990, a primer consisting of the adaptor sequence iii) and the ORF specific sequence SEQ ID NO: 12064 and a second primer consisting of the adaptor sequence iiii) and the ORF specific sequence SEQ ID NO: 12065 were used.

For amplification and cloning of Arabidopsis thaliana SEQ ID NO: 1298, a primer consisting of the adaptor sequence iii) and the ORF specific sequence SEQ ID NO: 1616 and a second primer consisting of the adaptor sequence iiii) and the ORF specific sequence SEQ ID NO: 1617 were used.

For amplification and cloning of Azotobacter vinelandii SEQ ID NO: 6040, a primer consisting of the adaptor sequence iii) and the ORF specific sequence SEQ ID NO: 6070 and a second primer consisting of the adaptor sequence iiii) and the ORF specific sequence SEQ ID NO: 6071 were used.

For amplification and cloning of Brassica napus SEQ ID NO: 140626, a primer consisting of the adaptor sequence iii) and the ORF specific sequence SEQ ID NO: 140762 and a second primer consisting of the adaptor sequence iiii) and the ORF specific sequence SEQ ID NO: 140763 were used.

For amplification and cloning of Glycine max SEQ ID NO: 69, a primer consisting of the adaptor sequence iii) and the ORF specific sequence SEQ ID NO: 343 and a second primer consisting of the adaptor sequence iiii) and the ORF specific sequence SEQ ID NO: 344 were used.

For amplification and cloning of Oryza sativa SEQ ID NO: 34301, a primer consisting of the adaptor sequence iii) and the ORF specific sequence SEQ ID NO: 34595 and a second primer consisting of the adaptor sequence iiii) and the ORF specific sequence SEQ ID NO: 34596 were used.

For amplification and cloning of Thermus thermophilus SEQ ID NO: 12698, a primer consisting of the adaptor sequence iii) and the ORF specific sequence SEQ ID NO: 12970 and a second primer consisting of the adaptor sequence iiii) and the ORF specific sequence SEQ ID NO: 12971 were used.

For amplification and cloning of Zea mays SEQ ID NO: 68413, a primer consisting of the adapfor sequence iii) and the ORF specific sequence SEQ ID NO: 68531 and a second primer consisting of the adaptor sequence iiii) and the ORF specific sequence SEQ ID NO: 68532 were used.

Following these examples every sequence disclosed in Table I, preferably column 5, especially the coding region thereof can be cloned by fusing the adaptor sequences to the respective specific primers sequences as disclosed in Table III, column 8 using the respective vectors shown in Table c.

[0496.1.7.17] to [0499.1.7.17] for the disclosure of these paragraphs see [0496.1.7.7] to [0499.1.7.7] above

### Example 11e

### Cloning of Inventive Sequences as Shown in Table I, Column 5 in the Different Expression Vectors

For cloning of for example SEQ ID NO: 7081 from Escherichia coli or any other ORF from Escherichia coli, Synechocystis sp., Azotobacter vinelandii, Thermus thermophilus, Arabidopsis thaliana, Brassica napus, Glycine max, Oryza sativa , or Zea mays the vector DNA was treated with the restriction enzymes Pacl and Ncol following the standard protocol (MBI Fermentas).

For cloning for example the ORFs of SEQ ID NO: 14171 from Saccharomyces cerevisiae or any other ORF from Saccharomyces cerevisiae into vectors containing the Resgen adaptor sequence the respective vector DNA was treated with the restriction enzyme Ncol. For cloning of for example ORFs SEQ ID NO: 63334 from Saccharomyces cerevisiae or any other ORF from Saccharomyces cerevisiae into vectors containing the Colic adaptor sequence, the respective vector DNA was treated with the restriction enzymes Pacl and Ncol following the standard protocol (MBI Fermentas).

In all cases the reaction was stopped by inactivation at 70° C. for 20 minutes and purified over QlAquick or NucleoSpin Extract II columns following the standard protocol (Qiagen or Macherey-Nagel).

Then the PCR-product representing the amplified ORF with the respective adapter sequences and the vector DNA were treated with T4 DNA polymerase according to the standard protocol (MBI Fermentas) to produce single stranded overhangs with the parameters 1 unit T4 DNA polymerase at 37° C. for 2-10 minutes for the vector and 1-2 u T4 DNA polymerase at 15-17° C. for 10-60 minutes for the PCR product representing SEQ ID NO: 7081.

The reaction was stopped by addition of high-salt buffer and purified over QlAquick or NucleoSpin Extract II columns following the standard protocol (Qiagen or Macherey-Nagel).

According to this example the skilled person is able to clone all sequences disclosed in Table I, preferably column 5 or column 8, especially the coding region thereof.

Approximately 30-60 ng of prepared vector and a defined amount of prepared amplificate were mixed and hybridized at 65° C. for 15 minutes followed by 37° C. 0.1° C/1 seconds, followed by 37° C. 10 minutes, followed by 0.1° C/1 seconds, then 4-10 ° C.

The ligated constructs were transformed in the same reaction vessel by addition of competent E. coli cells (strain DHSalpha) and incubation for 20 minutes at 1° C. followed by a heat shock for 90 seconds at 42° C. and cooling to 1-4° C. Then, complete medium (SOC) was added and the mixture was incubated for 45 minutes at 37° C. The entire mixture was subsequently plated onto an agar plate with 0.05 mg/ml kanamycin and incubated overnight at 37° C.

The outcome of the cloning step was verified by amplification with the aid of primers which bind upstream and downstream of the integration site, thus allowing the amplification of the insertion.

The amplifications were carried out as described in the protocol of Taq DNA polymerase (Gibco-BRL).

The amplification cycles were as follows:

1 cycle of 1-5 minutes at 94° C., followed by 35 cycles of in each case 15-60 seconds at 94° C., 15-60 seconds at 50-66° C. and 5-15 minutes at 72° C., followed by 1 cycle of 10 minutes at 72° C., then 4-16° C.

Several colonies were checked, but only one colony for which a PCR product of the expected size was detected was used in the following steps.

A portion of this positive colony was transferred into a reaction vessel filled with complete medium (LB) supplemented with kanamycin and incubated overnight at 37° C.

The plasmid preparation was carried out as specified in the Qiaprep or NucleoSpin Multi-96 Plus standard protocol (Qiagen or Macherey-Nagel).

[0501.1.7.17] to for the disclosure of these paragraphs see [0501.1.7.7] to [0503.1.7.17] above

Table d showing results of plant analyses

TABLE d

| SeqID | Target | Locus | Metabolite | Source | Promotor | Method | Min | Max |
|---|---|---|---|---|---|---|---|---|
| 142992 | non-targ | Atgols2 | raffinose | ARA_LEAF | p-Super | LC | 1186 | 8115 |
| 17451 | non-targ | At1g07430 | g-1-p | ARA_LEAF | p-PcUBI | GC | 27 | 90 |
| 17451 | non-targ | At1g07430 | my-inos | ARA_LEAF | p-PcUBI | GC | 25 | 96 |
| 140771 | non-targ | At1g19350 | g-1-p | ARA_LEAF | p-PcUBI | GC | 36 | 66 |
| 140771 | non-targ | At1g19350 | THP | ARA_LEAF | p-PcUBI | GC | 42 | 73 |
| 113651 | non-targ | At1g19670 | my-inos | ARA_LEAF | p-PcUBI | GC | 27 | 54 |
| 17701 | non-targ | At1g26830 | fructose | ARA_LEAF | p-PcUBI | GC | 107 | 121 |
| 17968 | non-targ | At1g36730 | fructose | ARA_LEAF | p-PcUBI | GC | 119 | 725 |
| 17968 | non-targ | At1g36730 | glucose | ARA_LEAF | p-PcUBI | GC | 95 | 312 |
| 17968 | non-targ | At1g36730 | raffinose | ARA_LEAF | p-PcUBI | LC | 57 | 150 |
| 18070 | non-targ | At1g43850 | sucrose | ARA_LEAF | p-PcUBI | GC | 40 | 87 |
| 140841 | non-targ | At1g60470 | raffinose | ARA_LEAF | p-Super | LC | 182 | 2025 |
| 19419 | non-targ | At1g72770 | glucose | ARA_LEAF | p-PcUBI | GC | 69 | 193 |
| 19419 | non-targ | At1g72770 | g-1-p | ARA_LEAF | p-PcUBI | GC | 19 | 102 |
| 19419 | non-targ | At1g72770 | my-inos | ARA_LEAF | p-PcUBI | GC | 49 | 123 |
| 19419 | non-targ | At1g72770 | raffinose | ARA_LEAF | p-PcUBI | LC | 55 | 339 |
| 19419 | non-targ | At1g72770 | sucrose | ARA_LEAF | p-PcUBI | GC | 35 | 82 |
| 141013 | non-targ | At1g78200 | sacch a | ARA_LEAF | p-PcUBI | LC | 49 | 143 |
| 19502 | non-targ | At2g17560 | fructose | ARA_LEAF | p-PcUBI | GC | 77 | 460 |
| 19502 | non-targ | At2g17560 | glucose | ARA_LEAF | p-PcUBI | GC | 67 | 304 |
| 19502 | non-targ | At2g17560 | g-1-p | ARA_LEAF | p-PcUBI | GC | 23 | 32 |
| 1298 | non-targ | At2g25070 | g-1-p | ARA_LEAF | p-PcUBI | GC | 19 | 37 |
| 20346 | non-targ | At2g30540 | sacch a | ARA_LEAF | p-PcUBI | LC | 44 | 162 |
| 20578 | non-targ | At2g34180 | fructose | ARA_LEAF | p-PcUBI | GC | 820 | 7343 |
| 20578 | non-targ | At2g34180 | glucose | ARA_LEAF | p-PcUBI | GC | 339 | 2993 |
| 20578 | non-targ | At2g34180 | sh-7-p | ARA_LEAF | p-PcUBI | GC | 190 | 580 |
| 133042 | non-targ | At2g37040 | g-1-p | ARA_LEAF | p-PcUBI | GC | 34 | 95 |
| 108727 | non-targ | At2g42830 | g-1-p | ARA_LEAF | p-PcUBI | GC | 33 | 85 |
| 1623 | non-targ | At2g45420 | my-inos | ARA_LEAF | p-PcUBI | GC | 28 | 34 |
| 21106 | non-targ | At2g46500 | g-1-p | ARA_LEAF | p-PcUBI | GC | 19 | 26 |
| 141637 | non-targ | At2g47180 | raffinose | ARA_LEAF | p-PcUBI | LC | 569 | 4907 |
| 141637 | non-targ | At2g47180 | raffinose | ARA_LEAF | Big35S | LC | 158 | 11191 |
| 68657 | non-targ | At3g02990 | fructose | ARA_LEAF | p-PcUBI | GC | 426 | 970 |
| 68657 | non-targ | At3g02990 | glucose | ARA_LEAF | p-PcUBI | GC | 58 | 465 |
| 68657 | non-targ | At3g02990 | my-inos | ARA_LEAF | p-PcUBI | GC | 29 | 46 |
| 68657 | non-targ | At3g02990 | raffinose | ARA_LEAF | p-PcUBI | LC | 117 | 1149 |
| 21497 | plastidic | At3g04050 | fructose | ARA_LEAF | p-PcUBI | GC | 192 | 299 |
| 22015 | non-targ | At3g06270 | glucose | ARA_LEAF | p-PcUBI | GC | 85 | 185 |
| 22015 | non-targ | At3g06270 | my-inos | ARA_LEAF | p-PcUBI | GC | 25 | 80 |
| 22249 | non-targ | At3g08710 | fructose | ARA_LEAF | p-PcUBI | GC | 93 | 182 |
| 22249 | non-targ | At3g08710 | glucose | ARA_LEAF | p-PcUBI | GC | 69 | 147 |
| 22249 | non-targ | At3g08710 | my-inos | ARA_LEAF | p-PcUBI | GC | 32 | 123 |
| 22699 | plastidic | At3g14230 | sacch a | ARA_LEAF | p-PcUBI | LC | 57 | 182 |
| 22921 | non-targ | At3g20910 | g-1-p | ARA_LEAF | p-PcUBI | GC | 18 | 45 |
| 22921 | non-targ | At3g20910 | raffinose | ARA_LEAF | p-PcUBI | LC | 55 | 114 |
| 1815 | non-targ | At3g23000 | fructose | ARA_LEAF | p-PcUBI | GC | 123 | 2049 |
| 1815 | non-targ | At3g23000 | glucose | ARA_LEAF | p-PcUBI | GC | 177 | 3162 |
| 1815 | non-targ | At3g23000 | sh-7-p | ARA_LEAF | p-PcUBI | GC | 116 | 571 |
| 68727 | non-targ | At3g26400 | fructose | ARA_LEAF | p-PcUBI | GC | 79 | 583 |
| 68727 | non-targ | At3g26400 | glucose | ARA_LEAF | p-PcUBI | GC | 112 | 264 |
| 68727 | non-targ | At3g26400 | raffinose | ARA_LEAF | p-PcUBI | LC | 89 | 129 |
| 141758 | non-targ | At3g59790 | my-inos | ARA_LEAF | p-PcUBI | GC | 33 | 77 |
| 2367 | non-targ | At3g61830 | sh-7-p | ARA_LEAF | p-PcUBI | GC | 54 | 87 |
| 2573 | non-targ | At3g62930 | fructose | ARA_LEAF | p-PcUBI | GC | 194 | 4745 |
| 2573 | non-targ | At3g62930 | glucose | ARA_LEAF | p-PcUBI | GC | 207 | 4142 |
| 2573 | non-targ | At3g62930 | g-1-p | ARA_LEAF | p-PcUBI | GC | 32 | 176 |
| 2573 | non-targ | At3g62930 | my-inos | ARA_LEAF | p-PcUBI | GC | 36 | 118 |
| 2573 | non-targ | At3g62930 | sh-7-p | ARA_LEAF | p-PcUBI | GC | 85 | 752 |
| 2573 | non-targ | At3g62930 | sucrose | ARA_LEAF | p-PcUBI | GC | 58 | 129 |
| 2935 | non-targ | At3g62950 | fructose | ARA_LEAF | p-PcUBI | GC | 623 | 3749 |
| 2935 | non-targ | At3g62950 | glucose | ARA_LEAF | p-PcUBI | GC | 867 | 3374 |
| 2935 | non-targ | At3g62950 | g-1-p | ARA_LEAF | p-PcUBI | GC | 31 | 141 |
| 2935 | non-targ | At3g62950 | my-inos | ARA_LEAF | p-PcUBI | GC | 46 | 95 |
| 2935 | non-targ | At3g62950 | sh-7-p | ARA_LEAF | p-PcUBI | GC | 167 | 848 |
| 2935 | non-targ | At3g62950 | sucrose | ARA_LEAF | p-PcUBI | GC | 49 | 114 |
| 109717 | non-targ | At4g09960 | g-1-p | ARA_LEAF | p-PcUBI | GC | 18 | 53 |
| 23482 | non-targ | At4g15660 | fructose | ARA_LEAF | p-PcUBI | GC | 321 | 3175 |
| 23482 | non-targ | At4g15660 | glucose | ARA_LEAF | p-PcUBI | GC | 231 | 2124 |
| 23482 | non-targ | At4g15660 | g-1-p | ARA_LEAF | p-PcUBI | GC | 107 | 199 |
| 23482 | non-targ | At4g15660 | my-inos | ARA_LEAF | p-PcUBI | GC | 97 | 241 |
| 23482 | non-targ | At4g15660 | sh-7-p | ARA_LEAF | p-PcUBI | GC | 183 | 692 |
| 23482 | non-targ | At4g15660 | sucrose | ARA_LEAF | p-PcUBI | GC | 41 | 106 |
| 3279 | non-targ | At4g15670 | fructose | ARA_LEAF | p-PcUBI | GC | 624 | 6054 |
| 3279 | non-targ | At4g15670 | glucose | ARA_LEAF | p-PcUBI | GC | 156 | 1091 |
| 3279 | non-targ | At4g15670 | g-1-p | ARA_LEAF | p-PcUBI | GC | 124 | 251 |
| 3279 | non-targ | At4g15670 | my-inos | ARA_LEAF | p-PcUBI | GC | 123 | 337 |
| 3279 | non-targ | At4g15670 | sh-7-p | ARA_LEAF | p-PcUBI | GC | 82 | 1306 |
| 3279 | non-targ | At4g15670 | sucrose | ARA_LEAF | p-PcUBI | GC | 38 | 91 |
| 23844 | non-targ | At4g15690 | fructose | ARA_LEAF | p-PcUBI | GC | 100 | 2161 |
| 23844 | non-targ | At4g15690 | glucose | ARA_LEAF | p-PcUBI | GC | 98 | 1660 |
| 23844 | non-targ | At4g15690 | g-1-p | ARA_LEAF | p-PcUBI | GC | 49 | 103 |
| 23844 | non-targ | At4g15690 | my-inos | ARA_LEAF | p-PcUBI | GC | 50 | 145 |
| 23844 | non-targ | At4g15690 | sucrose | ARA_LEAF | p-PcUBI | GC | 34 | 69 |
| 3654 | non-targ | At4g15700 | fructose | ARA_LEAF | p-PcUBI | GC | 722 | 1818 |
| 3654 | non-targ | At4g15700 | glucose | ARA_LEAF | p-PcUBI | GC | 469 | 1285 |
| 3654 | non-targ | At4g15700 | sh-7-p | ARA_LEAF | p-PcUBI | GC | 254 | 653 |
| 3654 | non-targ | At4g15700 | sucrose | ARA_LEAF | p-PcUBI | GC | 83 | 229 |
| 68849 | non-targ | At4g26080 | my-inos | ARA_LEAF | p-PcUBI | GC | 33 | 138 |
| 4102 | non-targ | At4g33040 | fructose | ARA_LEAF | p-PcUBI | GC | 79 | 387 |
| 4102 | non-targ | At4g33040 | glucose | ARA_LEAF | p-PcUBI | GC | 58 | 294 |
| 4102 | non-targ | At4g33040 | g-1-p | ARA_LEAF | p-PcUBI | GC | 19 | 65 |
| 4102 | non-targ | At4g33040 | my-inos | ARA_LEAF | p-PcUBI | GC | 50 | 110 |
| 4102 | non-targ | At4g33040 | sh-7-p | ARA_LEAF | p-PcUBI | GC | 47 | 108 |
| 4102 | non-targ | At4g33040 | sucrose | ARA_LEAF | p-PcUBI | GC | 28 | 74 |
| 24311 | non-targ | At4g34160 | g-1-p | ARA_LEAF | p-PcUBI | GC | 20 | 47 |
| 24311 | non-targ | At4g34160 | raffinose | ARA_LEAF | p-PcUBI | LC | 131 | 327 |
| 24311 | non-targ | At4g34160 | sucrose | ARA_LEAF | p-PcUBI | GC | 31 | 97 |
| 4348 | non-targ | At4g35310 | fructose | ARA_LEAF | p-PcUBI | GC | 112 | 350 |
| 4348 | non-targ | At4g35310 | g-1-p | ARA_LEAF | p-PcUBI | GC | 32 | 55 |
| 4348 | non-targ | At4g35310 | raffinose | ARA_LEAF | p-PcUBI | LC | 66 | 186 |
| 68987 | non-targ | At5g02760 | my-inos | ARA_LEAF | p-PcUBI | GC | 25 | 104 |
| 24438 | non-targ | At5g03720 | fructose | ARA_LEAF | p-PcUBI | GC | 134 | 1766 |
| 24438 | non-targ | At5g03720 | glucose | ARA_LEAF | p-PcUBI | GC | 106 | 2148 |
| 24438 | non-targ | At5g03720 | g-1-p | ARA_LEAF | p-PcUBI | GC | 28 | 106 |
| 24438 | non-targ | At5g03720 | my-inos | ARA_LEAF | p-PcUBI | GC | 92 | 427 |
| 24438 | non-targ | At5g03720 | raffinose | ARA_LEAF | p-PcUBI | LC | 1744 | 12039 |
| 24438 | non-targ | At5g03720 | sucrose | ARA_LEAF | p-PcUBI | GC | 35 | 100 |
| 24492 | non-targ | At5g07200 | my-inos | ARA_LEAF | p-PcUBI | GC | 24 | 47 |
| 142753 | non-targ | At5g10170 | my-inos | ARA_LEAF | p-PcUBI | GC | 83 | 386 |
| 142753 | non-targ | At5g10170 | raffinose | ARA_LEAF | p-PcUBI | LC | 72 | 155 |
| 69275 | non-targ | At5g14070 | glucose | ARA_LEAF | p-PcUBI | GC | 70 | 248 |
| 25283 | non-targ | At5g16650 | fructose | ARA_LEAF | p-PcUBI | GC | 80 | 153 |
| 4904 | non-targ | At5g18600 | fructose | ARA_LEAF | p-PcUBI | GC | 228 | 2679 |
| 4904 | non-targ | At5g18600 | glucose | ARA_LEAF | p-PcUBI | GC | 187 | 2197 |
| 4904 | non-targ | At5g18600 | my-inos | ARA_LEAF | p-PcUBI | GC | 46 | 76 |
| 4904 | non-targ | At5g18600 | sh-7-p | ARA_LEAF | p-PcUBI | GC | 149 | 336 |
| 4904 | non-targ | At5g18600 | sucrose | ARA_LEAF | p-PcUBI | GC | 40 | 141 |
| 25428 | non-targ | At5g39760 | fructose | ARA_LEAF | p-PcUBI | GC | 114 | 219 |
| 142929 | non-targ | At5g43630 | g-1-p | ARA_LEAF | p-PcUBI | GC | 65 | 74 |
| 142933 | non-targ | At5g54070 | my-inos | ARA_LEAF | p-PcUBI | GC | 30 | 65 |
| 142933 | non-targ | At5g54070 | raffinose | ARA_LEAF | p-PcUBI | LC | 68 | 383 |
| 5318 | non-targ | At5g57050 | g-1-p | ARA_LEAF | p-PcUBI | GC | 37 | 73 |
| 5318 | non-targ | At5g57050 | my-inos | ARA_LEAF | p-PcUBI | GC | 27 | 112 |
| 70006 | non-targ | At5g60110 | fructose | ARA_LEAF | p-PcUBI | GC | 90 | 185 |
| 70006 | non-targ | At5g60110 | glucose | ARA_LEAF | p-PcUBI | GC | 68 | 191 |
| 102899 | non-targ | At5g60440 | fructose | ARA_LEAF | p-PcUBI | GC | 91 | 939 |
| 102899 | non-targ | At5g60440 | raffinose | ARA_LEAF | p-PcUBI | LC | 93 | 149 |
| 102899 | non-targ | At5g60440 | sacch a | ARA_LEAF | p-PcUBI | LC | 43 | 64 |
| 122803 | non-targ | At5g66710 | g-1-p | ARA_LEAF | p-PcUBI | GC | 20 | 49 |
| 26120 | non-targ | Avin- | raffinose | ARA_LEAF | p-PcUBI | LC | 86 | 92 |
| | | DRAFT_1534 | | | | | | |
| 26196 | non-targ | Avin- | sucrose | ARA_LEAF | p-PcUBI | GC | 26 | 41 |
| | | DRAFT_1624 | | | | | | |
| 6040 | non-targ | Avin- | THP | ARA_LEAF | p-PcUBI | GC | 43 | 115 |
| | | DRAFT_2091 | | | | | | |
| 27021 | non-targ | Avin- | fructose | ARA_LEAF | p-PcUBI | GC | 131 | 486 |
| | | DRAFT_2344 | | | | | | |
| 27021 | non-targ | Avin- | glucose | ARA_LEAF | p-PcUBI | GC | 58 | 207 |
| | | DRAFT_2344 | | | | | | |
| 27882 | non-targ | Avin- | fructose | ARA_LEAF | p-PcUBI | GC | 77 | 744 |
| | | DRAFT_2521 | | | | | | |
| 27882 | non-targ | Avin- | sucrose | ARA_LEAF | p-PcUBI | GC | 24 | 95 |
| | | DRAFT_2521 | | | | | | |
| 28040 | non-targ | Avin- | g-1-p | ARA_LEAF | p-PcUBI | GC | 28 | 54 |
| | | DRAFT_2754 | | | | | | |
| 6075 | non-targ | Avin- | glucose | ARA_LEAF | p-PcUBI | GC | 77 | 249 |
| | | DRAFT_3028 | | | | | | |
| 6075 | non-targ | Avin- | g-1-p | ARA_LEAF | p-PcUBI | GC | 19 | 137 |
| | | DRAFT_3028 | | | | | | |
| 6075 | non-targ | Avin- | my-inos | ARA_LEAF | p-PcUBI | GC | 39 | 273 |
| | | DRAFT_3028 | | | | | | |
| 6075 | non-targ | Avin- | raffinose | ARA_LEAF | p-PcUBI | LC | 65 | 493 |
| | | DRAFT_3028 | | | | | | |
| 6075 | non-targ | Avin- | sh-7-p | ARA_LEAF | p-PcUBI | GC | 50 | 123 |
| | | DRAFT_3028 | | | | | | |
| 72346 | non-targ | Avin- | raffinose | ARA_LEAF | p-PcUBI | LC | 427 | 3237 |
| | | DRAFT_3135 | | | | | | |
| 29286 | non-targ | Avin- | fructose | ARA_LEAF | p-PcUBI | GC | 78 | 155 |
| | | DRAFT_3209 | | | | | | |
| 29286 | non-targ | Avin- | glucose | ARA_LEAF | p-PcUBI | GC | 72 | 167 |
| | | DRAFT_3209 | | | | | | |
| 29286 | non-targ | Avin- | g-1-p | ARA_LEAF | p-PcUBI | GC | 20 | 26 |
| | | DRAFT_3209 | | | | | | |
| 29500 | non-targ | Avin- | g-1-p | ARA_LEAF | p-PcUBI | GC | 19 | 61 |
| | | DRAFT_3253 | | | | | | |
| 111155 | non-targ | Avin- | my-inos | ARA_LEAF | p-PcUBI | GC | 27 | 74 |
| | | DRAFT_3577 | | | | | | |
| 31026 | non-targ | Avin- | fructose | ARA_LEAF | p-PcUBI | GC | 170 | 743 |
| | | DRAFT_3605 | | | | | | |
| 31026 | non-targ | Avin- | glucose | ARA_LEAF | p-PcUBI | GC | 67 | 683 |
| | | DRAFT_3605 | | | | | | |
| 31026 | non-targ | Avin- | sucrose | ARA_LEAF | p-PcUBI | GC | 46 | 102 |
| | | DRAFT_3605 | | | | | | |
| 143121 | non-targ | Avin- | raffinose | ARA_LEAF | p-PcUBI | LC | 55 | 99 |
| | | DRAFT_3616 | | | | | | |
| 114231 | non-targ | Avin- | my-inos | ARA_LEAF | p-PcUBI | GC | 44 | 288 |
| | | DRAFT_3629 | | | | | | |
| 114231 | non-targ | Avin- | raffinose | ARA_LEAF | p-PcUBI | LC | 107 | 400 |
| | | DRAFT_3629 | | | | | | |
| 143398 | non-targ | Avin- | my-inos | ARA_LEAF | p-PcUBI | GC | 29 | 72 |
| | | DRAFT_4265 | | | | | | |
| 143398 | non-targ | Avin- | raffinose | ARA_LEAF | p-PcUBI | LC | 62 | 126 |
| | | DRAFT_4265 | | | | | | |
| 73038 | non-targ | Avin- | raffinose | ARA_LEAF | p-PcUBI | LC | 74 | 180 |
| | | DRAFT_4420 | | | | | | |
| 73719 | non-targ | Avin- | fructose | ARA_LEAF | p-PcUBI | GC | 125 | 130 |
| | | DRAFT_4606 | | | | | | |
| 73719 | non-targ | Avin- | g-1-p | ARA_LEAF | p-PcUBI | GC | 24 | 34 |
| | | DRAFT_4606 | | | | | | |
| 73719 | non-targ | Avin- | sucrose | ARA_LEAF | p-PcUBI | GC | 28 | 74 |
| | | DRAFT_4606 | | | | | | |
| 102941 | non-targ | Avin- | THP | ARA_LEAF | p-PcUBI | GC | 44 | 86 |
| | | DRAFT_4847 | | | | | | |
| 6510 | non-targ | Avin- | fructose | ARA_LEAF | p-PcUBI | GC | 83 | 1502 |
| | | DRAFT_5103 | | | | | | |
| 6510 | non-targ | Avin- | glucose | ARA_LEAF | p-PcUBI | GC | 62 | 605 |
| | | DRAFT_5103 | | | | | | |
| 6510 | non-targ | Avin- | sucrose | ARA_LEAF | p-PcUBI | GC | 31 | 48 |
| | | DRAFT_5103 | | | | | | |
| 32037 | non-targ | Avin- | fructose | ARA_LEAF | p-PcUBI | GC | 82 | 370 |
| | | DRAFT_5246 | | | | | | |
| 32037 | non-targ | Avin- | glucose | ARA_LEAF | p-PcUBI | GC | 61 | 200 |
| | | DRAFT_5246 | | | | | | |
| 32037 | non-targ | Avin- | sucrose | ARA_LEAF | p-PcUBI | GC | 27 | 38 |
| | | DRAFT_5246 | | | | | | |
| 32308 | non-targ | Avin- | fructose | ARA_LEAF | p-PcUBI | GC | 186 | 188 |
| | | RAFT_5292 | | | | | | |
| 143495 | non-targ | Avin- | raffinose | ARA_LEAF | p-PcUBI | LC | 1093 | 3766 |
| | | DRAFT_5967 | | | | | | |
| 6674 | non-targ | Avin- | my-inos | ARA_LEAF | p-PcUBI | GC | 31 | 126 |
| | | DRAFT_6075 | | | | | | |
| 143614 | non-targ | AVIN- | THP | ARA_LEAF | p-PcUBI | GC | 33 | 77 |
| | | DRAFT_6261 | | | | | | |
| 74729 | non-targ | Avin- | fructose | ARA_LEAF | p-PcUBI | GC | 117 | 1104 |
| | | DRAFT_6679 | | | | | | |
| 74729 | non-targ | Avin- | glucose | ARA_LEAF | p-PcUBI | GC | 80 | 613 |
| | | DRAFT_6679 | | | | | | |
| 34044 | non-targ | Avin- | g-1-p | ARA_LEAF | p-PcUBI | GC | 53 | 114 |
| | | DRAFT_6700 | | | | | | |
| 34044 | non-targ | Avin- | THP | ARA_LEAF | p-PcUBI | GC | 60 | 108 |
| | | DRAFT_6700 | | | | | | |
| 34204 | non-targ | Avin- | raffinose | ARA_LEAF | p-PcUBI | LC | 62 | 341 |
| | | DRAFT_6864 | | | | | | |
| 34301 | non-targ | AX653549 | g-1-p | ARA_LEAF | p-PcUBI | GC | 28 | 50 |
| 34602 | non-targ | AY087308 | g-1-p | ARA_LEAF | p-PcUBI | GC | 28 | 47 |
| 34602 | non-targ | AY087308 | my-inos | ARA_LEAF | p-PcUBI | GC | 26 | 85 |
| 103433 | non-targ | AY623894 | g-1-p | ARA_LEAF | p-PcUBI | GC | 58 | 113 |
| 103433 | non-targ | AY623894 | THP | ARA_LEAF | p-PcUBI | GC | 33 | 167 |
| 34889 | plastidic | B0004 | glucose | ARA_LEAF | p-Super | GC | 62 | 423 |
| 34889 | plastidic | B0004 | sucrose | ARA_LEAF | p-Super | GC | 25 | 120 |
| 103958 | non-targ | B0050 | THP | ARA_LEAF | p-Super | GC | 33 | 63 |
| 35204 | non-targ | B0061 | raffinose | ARA_SEED_2 | p-USP | LC | 38 | 63 |
| 35482 | non-targ | B0124 | sucrose | ARA_LEAF | p-Super | GC | 27 | 83 |
| 7081 | non-targ | B0161 | g-1-p | ARA_LEAF | p-Super | GC | 24 | 53 |
| 7081 | non-targ | B0161 | my-inos | ARA_LEAF | p-Super | GC | 25 | 62 |
| 35590 | non-targ | B0221 | sucrose | ARA_LEAF | p-Super | GC | 25 | 89 |
| 143742 | non-targ | B0251 | g-1-p | ARA_LEAF | p-Super | GC | 20 | 59 |
| 121111 | non-targ | B0312 | fructose | ARA_LEAF | p-Super | GC | 76 | 386 |
| 35733 | plastidic | B0344 | melibiose | ARA_SEED_2 | p-USP | GC | 65 | 79 |
| 143761 | non-targ | B0362 | g-1-p | ARA_LEAF | p-Super | GC | 18 | 22 |
| 143771 | non-targ | B0376 | g-1-p | ARA_LEAF | p-Super | GC | 27 | 37 |
| 7333 | non-targ | B0449 | fructose | ARA_LEAF | p-Super | GC | 121 | 729 |
| 7333 | non-targ | B0449 | glucose | ARA_LEAF | p-Super | GC | 63 | 243 |
| 7686 | non-targ | B0486 | glucose | ARA_LEAF | p-Super | GC | 61 | 135 |
| 143817 | plastidic | B0488 | sacch a | ARA_LEAF | p-Super | LC | 57 | 74 |
| 134402 | non-targ | B0514 | galactinol | ARA_SEED_2 | p-USP | GC | 42 | 174 |
| 35967 | plastidic | B0593 | fructose | ARA_LEAF | p-Super | GC | 188 | 824 |
| 35967 | plastidic | B0593 | glucose | ARA_LEAF | p-Super | GC | 73 | 258 |
| 35967 | plastidic | B0593 | raffinose | ARA_LEAF | p-Super | LC | 70 | 127 |
| 35967 | plastidic | B0593 | sucrose | ARA_LEAF | p-Super | GC | 25 | 48 |
| 143901 | plastidic | B0644 | THP | ARA_LEAF | p-Super | GC | 40 | 79 |
| 143954 | non-targ | B0651 | sacch a | ARA_LEAF | p-Super | LC | 40 | 46 |
| 144207 | plastidic | B0674 | erythrol | ARA_SEED_2 | p-USP | GC | 239 | 281 |
| 93678 | non-targ | B0695 | my-inos | ARA_LEAF | p-Super | GC | 27 | 58 |
| 93793 | plastidic | B0728 | my-inos | ARA_LEAF | p-Super | GC | 26 | 47 |
| 36114 | non-targ | B0752 | fructose | ARA_LEAF | p-Super | GC | 77 | 258 |
| 36114 | non-targ | B0752 | glucose | ARA_LEAF | p-Super | GC | 76 | 641 |
| 36114 | non-targ | B0752 | g-1-p | ARA_LEAF | p-Super | GC | 30 | 78 |
| 36114 | non-targ | B0752 | raffinose | ARA_LEAF | p-Super | LC | 65 | 646 |
| 75807 | non-targ | B0801 | glucose | ARA_LEAF | p-Super | GC | 108 | 170 |
| 131359 | plastidic | B0856 | sh-7-p | ARA_LEAF | p-Super | GC | 74 | 127 |
| 36489 | non-targ | B0885 | raffinose | ARA_LEAF | p-Super | LC | 67 | 76 |
| 144479 | non-targ | B0970 | g-1-p | ARA_LEAF | p-Super | GC | 22 | 130 |
| 36809 | non-targ | B0980 | my-inos | ARA_SEED_2 | p-USP | GC | 26 | 130 |
| 7941 | non-targ | B1003 | fructose | ARA_LEAF | p-Super | GC | 278 | 1463 |
| 7941 | non-targ | B1003 | glucose | ARA_LEAF | p-Super | GC | 44 | 339 |
| 7941 | non-targ | B1003 | g-1-p | ARA_LEAF | p-Super | GC | 30 | 102 |
| 7941 | non-targ | B1003 | sh-7-p | ARA_LEAF | p-Super | GC | 62 | 799 |
| 7941 | non-targ | B1003 | sucrose | ARA_LEAF | p-Super | GC | 57 | 120 |
| 124051 | plastidic | B1046 | THP | ARA_LEAF | p-Super | GC | 33 | 49 |
| 76032 | plastidic | B1065 | g-1-p | ARA_LEAF | p-Super | GC | 19 | 35 |
| 76032 | plastidic | B1065 | sucrose | ARA_LEAF | p-Super | GC | 32 | 37 |
| 114979 | non-targ | B1102 | fructose | ARA_LEAF | p-Super | GC | 87 | 189 |
| 144611 | plastidic | B1131 | g-1-p | ARA_LEAF | p-Super | GC | 18 | 23 |
| 36971 | plastidic | B1136 | glucose | ARA_LEAF | p-Super | GC | 50 | 104 |
| 37390 | non-targ | B1137 | g-1-p | ARA_LEAF | p-Super | GC | 20 | 65 |
| 37400 | non-targ | B1186 | g-1-p | ARA_LEAF | p-Super | GC | 24 | 126 |
| 144857 | non-targ | B1214 | g-1-p | ARA_LEAF | p-Super | GC | 43 | 71 |
| 144857 | non-targ | B1214 | THP | ARA_LEAF | p-Super | GC | 40 | 58 |
| 112072 | non-targ | B1219 | g-1-p | ARA_LEAF | p-Super | GC | 40 | 58 |
| 112072 | non-targ | B1219 | my-inos | ARA_LEAF | p-Super | GC | 36 | 38 |
| 112072 | non-targ | B1219 | THP | ARA_LEAF | p-Super | GC | 35 | 76 |
| 112111 | non-targ | B1234 | my-inos | ARA_LEAF | p-Super | GC | 26 | 38 |
| 98778 | plastidic | B1249 | galactinol | ARA_SEED_2 | p-USP | GC | 40 | 153 |
| 98778 | plastidic | B1249 | galactitol | ARA_SEED_2 | p-USP | GC | 65 | 272 |
| 98778 | plastidic | B1249 | sorbitol | ARA_SEED_2 | p-USP | GC | 65 | 272 |
| 144894 | non-targ | B1254 | THP | ARA_LEAF | p-Super | GC | 37 | 56 |
| 37483 | non-targ | B1255 | fructose | ARA_LEAF | p-Super | GC | 80 | 727 |
| 37483 | non-targ | B1255 | glucose | ARA_LEAF | p-Super | GC | 66 | 597 |
| 37483 | non-targ | B1255 | raffinose | ARA_LEAF | p-Super | LC | 82 | 369 |
| 37483 | non-targ | B1255 | sucrose | ARA_LEAF | p-Super | GC | 33 | 52 |
| 76157 | plastidic | B1262 | my-inos | ARA_SEED_2 | p-USP | GC | 32 | 577 |
| 134655 | non-targ | B1285 | fructose | ARA_LEAF | p-Super | GC | 71 | 127 |
| 124557 | non-targ | B1356 | g-1-p | ARA_LEAF | p-Super | GC | 19 | 20 |
| 145066 | non-targ | B1357 | g-1-p | ARA_LEAF | p-Super | GC | 20 | 44 |
| 134697 | non-targ | B1390 | glucose | ARA_LEAF | p-Super | GC | 57 | 120 |
| 131489 | non-targ | B1391 | THP | ARA_LEAF | p-Super | GC | 43 | 95 |
| 145091 | plastidic | B1410 | sh-7-p | ARA_LEAF | p-Super | GC | 43 | 112 |
| 134756 | non-targ | B1425 | my-inos | ARA_LEAF | p-Super | GC | 25 | 36 |
| 38266 | plastidic | B1431 | g-1-p | ARA_LEAF | p-Super | GC | 31 | 52 |
| 38266 | plastidic | B1431 | sh-7-p | ARA_LEAF | p-Super | GC | 32 | 52 |
| 38266 | plastidic | B1431 | THP | ARA_LEAF | p-Super | GC | 37 | 89 |
| 38289 | non-targ | B1445 | THP | ARA_LEAF | p-Super | GC | 47 | 50 |
| 115056 | non-targ | B1470 | g-1-p | ARA_LEAF | p-Super | GC | 18 | 44 |
| 7947 | non-targ | B1522 | fructose | ARA_LEAF | p-Super | GC | 83 | 364 |
| 7947 | non-targ | B1522 | g-1-p | ARA_LEAF | p-Super | GC | 20 | 41 |
| 145142 | plastidic | B1556 | g-1-p | ARA_LEAF | p-Super | GC | 35 | 57 |
| 145142 | plastidic | B1556 | sh-7-p | ARA_LEAF | p-Super | GC | 86 | 382 |
| 78138 | non-targ | B1583 | g-1-p | ARA_LEAF | p-Super | GC | 23 | 46 |
| 78154 | non-targ | B1589 | g-1-p | ARA_LEAF | p-Super | GC | 50 | 114 |
| 78154 | non-targ | B1589 | THP | ARA_LEAF | p-Super | GC | 33 | 122 |
| 38300 | non-targ | B1597 | fructose | ARA_LEAF | p-Super | GC | 78 | 250 |
| 38300 | non-targ | B1597 | glucose | ARA_LEAF | p-Super | GC | 86 | 187 |
| 38300 | non-targ | B1597 | g-1-p | ARA_LEAF | p-Super | GC | 47 | 249 |
| 38300 | non-targ | B1597 | my-inos | ARA_LEAF | p-Super | GC | 33 | 81 |
| 38300 | non-targ | B1597 | raffinose | ARA_LEAF | p-Super | LC | 65 | 199 |
| 124571 | non-targ | B1621 | fructose | ARA_LEAF | p-Super | GC | 107 | 424 |
| 124571 | non-targ | B1621 | glucose | ARA_LEAF | p-Super | GC | 77 | 119 |
| 38345 | non-targ | B1627 | glucose | ARA_LEAF | p-Super | GC | 72 | 186 |
| 38345 | non-targ | B1627 | g-1-p | ARA_LEAF | p-Super | GC | 32 | 78 |
| 38345 | non-targ | B1627 | THP | ARA_LEAF | p-Super | GC | 55 | 78 |
| 78265 | non-targ | B1651 | my-inos | ARA_LEAF | p-Super | GC | 25 | 41 |
| 78753 | non-targ | B1670 | my-inos | ARA_LEAF | p-Super | GC | 30 | 45 |
| 78852 | non-targ | B1755 | fructose | ARA_LEAF | p-Super | GC | 83 | 94 |
| 78883 | non-targ | B1791 | my-inos | ARA_LEAF | p-Super | GC | 25 | 344 |
| 99196 | non-targ | B1796 | fructose | ARA_LEAF | p-Super | GC | 85 | 93 |
| 145180 | non-targ | B1812 | my-inos | ARA_LEAF | p-Super | GC | 33 | 49 |
| 8033 | plastidic | B1814 | galactitol | ARA_SEED_2 | p-USP | GC | 55 | 2661 |
| 8033 | plastidic | B1814 | sorbitol | ARA_SEED_2 | p-USP | GC | 55 | 2661 |
| 38573 | non-targ | B1845 | my-inos | ARA_LEAF | p-Super | GC | 26 | 51 |
| 8363 | plastidic | B1854 | erythrol | ARA_SEED_2 | p-USP | GC | 140 | 330 |
| 8363 | plastidic | B1854 | my-inos | ARA_SEED_2 | p-USP | GC | 44 | 81 |
| 8363 | plastidic | B1854 | sorbitol | ARA_SEED_2 | p-USP | LC | 200 | 414 |
| 134783 | non-targ | B1865 | fructose | ARA_SEED_2 | p-USP | GC | 32 | 105 |
| 145411 | non-targ | B1896 | g-1-p | ARA_LEAF | p-Super | GC | 19 | 74 |
| 145411 | non-targ | B1896 | trehalose | ARA_LEAF | p-Super | GC | 294 | 441 |
| 79056 | plastidic | B1897 | erythrol | ARA_SEED_2 | p-USP | GC | 139 | 3201 |
| 79056 | non-targ | B1897 | fructose | ARA_LEAF | p-Super | GC | 202 | 2339 |
| 79056 | non-targ | B1897 | glucose | ARA_LEAF | p-Super | GC | 120 | 1091 |
| 79056 | non-targ | B1897 | sucrose | ARA_LEAF | p-Super | GC | 38 | 103 |
| 38767 | non-targ | B1898 | raffinose | ARA_LEAF | p-Super | LC | 55 | 329 |
| 131799 | non-targ | B1932 | g-1-p | ARA_LEAF | p-Super | GC | 19 | 107 |
| 131799 | non-targ | B1932 | THP | ARA_LEAF | p-Super | GC | 33 | 84 |
| 134864 | non-targ | B1938 | glucose | ARA_LEAF | p-Super | GC | 65 | 201 |
| 134864 | non-targ | B1938 | my-inos | ARA_LEAF | p-Super | GC | 28 | 190 |
| 79217 | non-targ | B2027 | fructose | ARA_LEAF | p-Super | GC | 156 | 232 |
| 8920 | non-targ | B2032 | glucose | ARA_LEAF | p-Super | GC | 61 | 157 |
| 8920 | non-targ | B2032 | my-inos | ARA_LEAF | p-Super | GC | 27 | 212 |
| 112193 | non-targ | B2043 | g-1-p | ARA_LEAF | p-Super | GC | 18 | 35 |
| 104277 | non-targ | B2053 | fucose | ARA_LEAF | p-Super | GC | 1022 | 6264 |
| 38947 | non-targ | B2063 | fructose | ARA_LEAF | p-Super | GC | 78 | 1849 |
| 38947 | non-targ | B2063 | g-1-p | ARA_LEAF | p-Super | GC | 32 | 87 |
| 38947 | non-targ | B2063 | raffinose | ARA_LEAF | p-Super | LC | 59 | 242 |
| 38947 | non-targ | B2063 | sucrose | ARA_LEAF | p-Super | GC | 24 | 87 |
| 38947 | non-targ | B2063 | THP | ARA_LEAF | p-Super | GC | 37 | 111 |
| 39002 | non-targ | B2107 | g-1-p | ARA_LEAF | p-Super | GC | 37 | 71 |
| 39002 | non-targ | B2107 | THP | ARA_LEAF | p-Super | GC | 39 | 94 |
| 125085 | plastidic | B2139 | g-1-p | ARA_LEAF | p-Super | GC | 32 | 44 |
| 79279 | plastidic | B2242 | erythrol | ARA_SEED_2 | p-USP | GC | 244 | 808 |
| 99620 | non-targ | B2285 | fructose | ARA_SEED_2 | p-USP | GC | 26 | 113 |
| 145692 | non-targ | B2290 | galactinol | ARA_SEED_2 | p-USP | GC | 54 | 149 |
| 9156 | non-targ | B2345 | my-inos | ARA_LEAF | p-Super | GC | 25 | 60 |
| 9156 | non-targ | B2345 | THP | ARA_LEAF | p-Super | GC | 41 | 68 |
| 39219 | non-targ | B2360 | verbascose | ARA_LEAF | p-Super | LC | 67 | 1435 |
| 39219 | non-targ | B2360 | THP | ARA_LEAF | p-Super | GC | 38 | 65 |
| 146204 | non-targ | B2361 | g-1-p | ARA_LEAF | p-Super | GC | 31 | 46 |
| 146204 | non-targ | B2361 | THP | ARA_LEAF | p-Super | GC | 33 | 79 |
| 146238 | non-targ | B2389 | sucrose | ARA_LEAF | p-Super | GC | 32 | 83 |
| 39237 | non-targ | B2399 | fructose | ARA_LEAF | p-Super | GC | 147 | 1537 |
| 39237 | non-targ | B2399 | glucose | ARA_LEAF | p-Super | GC | 97 | 2165 |
| 39237 | non-targ | B2399 | g-1-p | ARA_LEAF | p-Super | GC | 68 | 231 |
| 39237 | non-targ | B2399 | my-inos | ARA_LEAF | p-Super | GC | 33 | 1261 |
| 39237 | non-targ | B2399 | raffinose | ARA_LEAF | p-Super | LC | 862 | 3481 |
| 39237 | non-targ | B2399 | sh-7-p | ARA_LEAF | p-Super | GC | 51 | 583 |
| 39237 | non-targ | B2399 | sucrose | ARA_LEAF | p-Super | GC | 26 | 131 |
| 39255 | non-targ | B2405 | fructose | ARA_LEAF | p-Super | GC | 77 | 106 |
| 39255 | non-targ | B2405 | raffinose | ARA_LEAF | p-Super | LC | 58 | 61 |
| 39300 | non-targ | B2414 | my-inos | ARA_LEAF | p-Super | GC | 26 | 55 |
| 39300 | non-targ | B2414 | verbascose | ARA_LEAF | p-Super | LC | 49 | 743 |
| 146261 | non-targ | B2466 | g-1-p | ARA_LEAF | p-Super | GC | 35 | 116 |
| 146261 | non-targ | B2466 | my-inos | ARA_LEAF | p-Super | GC | 33 | 154 |
| 146261 | non-targ | B2466 | THP | ARA_LEAF | p-Super | GC | 37 | 62 |
| 40329 | non-targ | B2474 | g-1-p | ARA_LEAF | p-Super | GC | 23 | 57 |
| 40383 | non-targ | B2541 | raffinose | ARA_LEAF | p-Super | LC | 95 | 173 |
| 99898 | non-targ | B2546 | sucrose | ARA_LEAF | p-Super | GC | 23 | 38 |
| 40665 | non-targ | B2613 | g-1-p | ARA_LEAF | p-Super | GC | 18 | 42 |
| 40665 | non-targ | B2613 | my-inos | ARA_LEAF | p-Super | GC | 26 | 43 |
| 40726 | non-targ | B2634 | fructose | ARA_LEAF | p-Super | GC | 110 | 4796 |
| 40726 | non-targ | B2634 | my-inos | ARA_LEAF | p-Super | GC | 27 | 1006 |
| 40726 | non-targ | B2634 | sucrose | ARA_LEAF | p-Super | GC | 28 | 696 |
| 146288 | non-targ | B2653 | g-1-p | ARA_LEAF | p-Super | GC | 31 | 45 |
| 146288 | non-targ | B2653 | sh-7-p | ARA_LEAF | p-Super | GC | 57 | 70 |
| 146288 | non-targ | B2653 | THP | ARA_LEAF | p-Super | GC | 37 | 69 |
| 125226 | non-targ | B2654 | g-1-p | ARA_LEAF | p-Super | GC | 39 | 54 |
| 125226 | non-targ | B2654 | THP | ARA_LEAF | p-Super | GC | 34 | 70 |
| 146294 | non-targ | B2670 | g-1-p | ARA_LEAF | p-Super | GC | 34 | 97 |
| 9244 | non-targ | B2673 | g-1-p | ARA_LEAF | p-Super | GC | 31 | 60 |
| 9244 | non-targ | B2673 | THP | ARA_LEAF | p-Super | GC | 40 | 56 |
| 115655 | non-targ | B2682 | my-inos | ARA_LEAF | p-Super | GC | 25 | 113 |
| 100105 | non-targ | B2699 | g-1-p | ARA_LEAF | p-Super | GC | 65 | 214 |
| 80756 | non-targ | B2701 | g-1-p | ARA_LEAF | p-Super | GC | 36 | 59 |
| 40741 | non-targ | B2714 | my-inos | ARA_LEAF | p-Super | GC | 29 | 45 |
| 80906 | non-targ | B2739 | fructose | ARA_LEAF | p-Super | GC | 76 | 544 |
| 80906 | non-targ | B2739 | glucose | ARA_LEAF | p-Super | GC | 77 | 212 |
| 81075 | non-targ | B2747 | glucose | ARA_LEAF | p-Super | GC | 81 | 417 |
| 81075 | non-targ | B2747 | my-inos | ARA_LEAF | p-Super | GC | 34 | 238 |
| 81075 | non-targ | B2747 | raffinose | ARA_LEAF | p-Super | LC | 64 | 612 |
| 81451 | non-targ | B2762 | g-1-p | ARA_LEAF | p-Super | GC | 18 | 30 |
| 81451 | non-targ | B2762 | THP | ARA_LEAF | p-Super | GC | 35 | 58 |
| 115719 | non-targ | B2790 | fructose | ARA_LEAF | p-Super | GC | 134 | 138 |
| 115719 | non-targ | B2790 | glucose | ARA_LEAF | p-Super | GC | 60 | 155 |
| 115719 | non-targ | B2790 | g-1-p | ARA_LEAF | p-Super | GC | 32 | 81 |
| 115719 | non-targ | B2790 | my-inos | ARA_LEAF | p-Super | GC | 25 | 116 |
| 115719 | non-targ | B2790 | THP | ARA_LEAF | p-Super | GC | 43 | 90 |
| 81576 | non-targ | B2818 | fructose | ARA_LEAF | p-Super | GC | 89 | 1378 |
| 81576 | non-targ | B2818 | g-1-p | ARA_LEAF | p-Super | GC | 25 | 43 |
| 81576 | non-targ | B2818 | my-inos | ARA_LEAF | p-Super | GC | 25 | 67 |
| 81935 | non-targ | B2839 | raffinose | ARA_LEAF | p-Super | LC | 121 | 159 |
| 9333 | non-targ | B2923 | glucose | ARA_LEAF | p-Super | GC | 77 | 194 |
| 9333 | non-targ | B2923 | my-inos | ARA_LEAF | p-Super | GC | 24 | 107 |
| 146327 | non-targ | B2932 | my-inos | ARA_LEAF | p-Super | GC | 26 | 61 |
| 104660 | plastidic | B3112 | erythrol | ARA_SEED_2 | p-USP | GC | 147 | 413 |
| 42477 | non-targ | B3151 | my-inos | ARA_LEAF | p-Super | GC | 33 | 139 |
| 42477 | non-targ | B3151 | raffinose | ARA_LEAF | p-Super | LC | 65 | 188 |
| 42477 | non-targ | B3151 | sucrose | ARA_LEAF | p-Super | GC | 25 | 64 |
| 82510 | plastidic | B3221 | fructose | ARA_LEAF | p-Super | GC | 40 | 58 |
| 82510 | plastidic | B3221 | glucose | ARA_LEAF | p-Super | GC | 58 | 73 |
| 82510 | plastidic | B3221 | THP | ARA_LEAF | p-Super | GC | 44 | 62 |
| 9470 | non-targ | B3246 | my-inos | ARA_LEAF | p-Super | GC | 311 | 476 |
| 9492 | non-targ | B3256 | my-inos | ARA_SEED_2 | p-USP | GC | 25 | 229 |
| 42502 | non-targ | B3262 | fructose | ARA_LEAF | p-Super | GC | 79 | 736 |
| 42502 | non-targ | B3262 | glucose | ARA_LEAF | p-Super | GC | 58 | 334 |
| 42502 | non-targ | B3262 | g-1-p | ARA_LEAF | p-Super | GC | 43 | 64 |
| 42502 | non-targ | B3262 | raffinose | ARA_LEAF | p-Super | LC | 186 | 930 |
| 42502 | non-targ | B3262 | sucrose | ARA_LEAF | p-Super | GC | 30 | 67 |
| 146364 | plastidic | B3337 | glucose | ARA_LEAF | p-Super | GC | 38 | 48 |
| 10104 | non-targ | B3346 | my-inos | ARA_LEAF | p-Super | GC | 30 | 74 |
| 10104 | non-targ | B3346 | raffinose | ARA_LEAF | p-Super | LC | 73 | 370 |
| 10104 | non-targ | B3346 | THP | ARA_LEAF | p-Super | GC | 34 | 74 |
| 82866 | non-targ | B3403 | fructose | ARA_LEAF | p-Super | GC | 77 | 143 |
| 82866 | non-targ | B3403 | glucose | ARA_LEAF | p-Super | GC | 79 | 87 |
| 42579 | non-targ | B3427 | THP | ARA_LEAF | p-Super | GC | 39 | 125 |
| 94051 | mitochondrial | B3429 | fructose | ARA_LEAF | p-Super | GC | 79 | 211 |
| 94051 | mitochondrial | B3429 | glucose | ARA_LEAF | p-Super | GC | 39 | 65 |
| 94380 | non-targ | B3442 | my-inos | ARA_LEAF | p-Super | GC | 27 | 82 |
| 146440 | plastidic | B3443 | THP | ARA_LEAF | p-Super | GC | 44 | 69 |
| 116172 | plastidic | B3451 | sacch a | ARA_LEAF | p-Super | LC | 53 | 59 |
| 146472 | non-targ | B3457 | g-1-p | ARA_LEAF | p-Super | GC | 28 | 51 |
| 146472 | non-targ | B3457 | my-inos | ARA_LEAF | p-Super | GC | 24 | 54 |
| 146646 | plastidic | B3519 | fructose | ARA_SEED_2 | p-USP | GC | 24 | 71 |
| 146646 | plastidic | B3519 | galactinol | ARA_SEED_2 | p-USP | GC | 40 | 107 |
| 146732 | non-targ | B3600 | fructose | ARA_SEED_2 | p-Super | GC | 69 | 185 |
| 146732 | non-targ | B3600 | mannitol | ARA_LEAF | p-Super | LC | 422 | 16165 |
| 146732 | non-targ | B3600 | sorbitol | ARA_SEED_2 | p-Super | LC | 3140 | 12520 |
| 94386 | non-targ | B3774 | galactinol | ARA_SEED_2 | p-USP | GC | 37 | 119 |
| 136098 | non-targ | B3777 | my-inos | ARA_LEAF | p-Super | GC | 26 | 50 |
| 10708 | non-targ | B3817 | my-inos | ARA_LEAF | p-Super | GC | 38 | 203 |
| 10708 | non-targ | B3817 | raffinose | ARA_LEAF | p-Super | LC | 220 | 467 |
| 146834 | non-targ | B3818 | my-inos | ARA_LEAF | p-Super | GC | 31 | 45 |
| 10726 | non-targ | B3823 | my-inos | ARA_LEAF | p-Super | GC | 29 | 113 |
| 101327 | plastidic | B3870 | my-inos | ARA_SEED_2 | p-USP | GC | 26 | 69 |
| 101327 | plastidic | B3870 | verbascose | ARA_SEED_2 | p-USP | LC | 67 | 181 |
| 83821 | plastidic | B3872 | g-1-p | ARA_LEAF | p-Super | GC | 27 | 59 |
| 44372 | non-targ | B3989 | fructose | ARA_LEAF | p-Super | GC | 105 | 1389 |
| 44372 | non-targ | B3989 | glucose | ARA_LEAF | p-Super | GC | 90 | 370 |
| 44372 | non-targ | B3989 | g-1-p | ARA_LEAF | p-Super | GC | 18 | 43 |
| 44372 | non-targ | B3989 | raffinose | ARA_LEAF | p-Super | LC | 95 | 272 |
| 44372 | non-targ | B3989 | sucrose | ARA_LEAF | p-Super | GC | 37 | 65 |
| 44378 | non-targ | B4012 | galactinol | ARA_SEED_2 | p-USP | GC | 37 | 102 |
| 10740 | non-targ | B4029 | fructose | ARA_LEAF | p-Super | GC | 83 | 2120 |
| 10740 | non-targ | B4029 | glucose | ARA_LEAF | p-Super | GC | 72 | 1072 |
| 10740 | non-targ | B4029 | sh-7-p | ARA_LEAF | p-Super | GC | 46 | 404 |
| 146848 | plastidic | B4039 | g-1-p | ARA_LEAF | p-Super | GC | 20 | 54 |
| 146848 | plastidic | B4039 | THP | ARA_LEAF | p-Super | GC | 33 | 87 |
| 106532 | non-targ | B4050 | fructose | ARA_LEAF | p-Super | GC | 114 | 1402 |
| 106532 | non-targ | B4050 | glucose | ARA_LEAF | p-Super | GC | 102 | 725 |
| 106532 | non-targ | B4050 | my-inos | ARA_LEAF | p-Super | GC | 27 | 309 |
| 94542 | non-targ | B4056 | fructose | ARA_LEAF | p-Super | GC | 140 | 163 |
| 126421 | plastidic | B4069 | galactinol | ARA_SEED_2 | p-USP | GC | 47 | 118 |
| 146921 | non-targ | B4189 | g-1-p | ARA_LEAF | p-Super | GC | 31 | 67 |
| 146921 | non-targ | B4189 | THP | ARA_LEAF | p-Super | GC | 43 | 74 |
| 94735 | non-targ | B4250 | my-inos | ARA_LEAF | p-Super | GC | 31 | 169 |
| 94735 | non-targ | B4250 | raffinose | ARA_LEAF | p-Super | LC | 188 | 278 |
| 45321 | non-targ | B4256 | fructose | ARA_LEAF | p-Super | GC | 219 | 2357 |
| 45321 | non-targ | B4256 | glucose | ARA_LEAF | p-Super | GC | 88 | 504 |
| 45394 | non-targ | B4321 | fructose | ARA_LEAF | p-Super | GC | 104 | 177 |
| 45556 | non-targ | B4384 | galactinol | ARA_SEED_2 | p-USP | GC | 56 | 127 |
| 84079 | plastidic | D90900 | my-inos | ARA_LEAF | p-PcUBI | GC | 37 | 67 |
| 10811 | non-targ | GM02LC12622 | fructose | ARA_LEAF | p-PcUBI | GC | 222 | 4601 |
| 10811 | non-targ | GM02LC12622 | glucose | ARA_LEAF | p-PcUBI | GC | 88 | 366 |
| 10811 | non-targ | GM02LC12622 | g-1-p | ARA_LEAF | p-PcUBI | GC | 26 | 99 |
| 10811 | non-targ | GM02LC12622 | my-inos | ARA_LEAF | p-PcUBI | GC | 25 | 119 |
| 10811 | non-targ | GM02LC12622 | sucrose | ARA_LEAF | p-PcUBI | GC | 30 | 134 |
| 46751 | non-targ | GM02LC19289 | g-1-p | ARA_LEAF | p-PcUBI | GC | 35 | 110 |
| 46751 | non-targ | GM02LC19289 | THP | ARA_LEAF | p-PcUBI | GC | 39 | 119 |
| 84198 | non-targ | GM02LC21368 | fructose | ARA_LEAF | p-PcUBI | GC | 82 | 665 |
| 116312 | non-targ | GM02LC38418 | g-1-p | ARA_LEAF | p-PcUBI | GC | 46 | 57 |
| 46850 | non-targ | GM02LC44512 | g-1-p | ARA_LEAF | p-PcUBI | GC | 47 | 85 |
| 46850 | non-targ | GM02LC44512 | raffinose | ARA_LEAF | p-PcUBI | LC | 60 | 997 |
| 46850 | non-targ | GM02LC44512 | sucrose | ARA_LEAF | p-PcUBI | GC | 28 | 94 |
| 47026 | non-targ | GM02LC46 | THP | ARA_LEAF | p-PcUBI | GC | 35 | 69 |
| 47105 | non-targ | GM02LC6021 | THP | ARA_LEAF | p-PcUBI | GC | 38 | 92 |
| 146939 | plastidic | Sll0222 | my-inos | ARA_LEAF | p-PcUBI | GC | 26 | 48 |
| 49143 | plastidic | Sll0248 | verbascose | ARA_LEAF | p-PcUBI | LC | 88 | 378 |
| 49800 | mitochondrial | Sll0254 | sucrose | ARA_LEAF | p-PcUBI | GC | 25 | 62 |
| 84865 | plastidic | Sll0281 | verbascose | ARA_LEAF | p-PcUBI | LC | 107 | 525 |
| 50070 | non-targ | Sll0354 | my-inos | ARA_LEAF | p-PcUBI | GC | 25 | 38 |
| 106920 | mitochondrial | Sll0368 | my-inos | ARA_LEAF | p-PcUBI | GC | 25 | 77 |
| 94942 | non-targ | Sll0418 | g-1-p | ARA_LEAF | p-PcUBI | GC | 43 | 164 |
| 94942 | mitochondrial | Sll0418 | my-inos | ARA_LEAF | p-PcUBI | GC | 33 | 56 |
| 94942 | non-targ | Sll0418 | my-inos | ARA_LEAF | p-PcUBI | GC | 26 | 38 |
| 94942 | non-targ | Sll0418 | sacch a | ARA_LEAF | p-PcUBI | LC | 51 | 100 |
| 94942 | non-targ | Sll0418 | THP | ARA_LEAF | p-PcUBI | GC | 37 | 157 |
| 50104 | non-targ | Sll0420 | g-1-p | ARA_LEAF | p-PcUBI | GC | 23 | 36 |
| 85510 | plastidic | Sll0593 | g-1-p | ARA_LEAF | p-PcUBI | GC | 35 | 72 |
| 146945 | non-targ | Sll0643 | sacch a | ARA_LEAF | p-PcUBI | LC | 46 | 111 |
| 101707 | non-targ | Sll0833 | g-1-p | ARA_LEAF | p-PcUBI | GC | 33 | 132 |
| 101707 | non-targ | Sll0833 | THP | ARA_LEAF | p-PcUBI | GC | 39 | 122 |
| 52246 | mitochondrial | Sll0934 | g-1-p | ARA_LEAF | p-PcUBI | GC | 39 | 58 |
| 52364 | non-targ | Sll0945 | fructose | ARA_LEAF | p-PcUBI | GC | 94 | 495 |
| 52364 | non-targ | Sll0945 | sucrose | ARA_LEAF | p-PcUBI | GC | 30 | 82 |
| 53189 | non-targ | Sll1185 | g-1-p | ARA_LEAF | p-PcUBI | GC | 32 | 41 |
| 147208 | non-targ | SLL1308 | THP | ARA_LEAF | p-PcUBI | GC | 34 | 69 |
| 53456 | non-targ | Sll1393 | raffinose | ARA_LEAF | p-PcUBI | LC | 71 | 157 |
| 95047 | non-targ | Sll1498 | my-inos | ARA_LEAF | p-PcUBI | GC | 24 | 28 |
| 95047 | non-targ | Sll1498 | THP | ARA_LEAF | p-PcUBI | GC | 38 | 68 |
| 54897 | non-targ | Sll1676 | my-inos | ARA_LEAF | p-PcUBI | GC | 20 | 24 |
| 55063 | plastidic | Sll1682 | THP | ARA_LEAF | p-PcUBI | GC | 33 | 65 |
| 55379 | non-targ | Sll1761 | glucose | ARA_LEAF | p-PcUBI | GC | 62 | 157 |
| 86447 | plastidic | Sll1848 | glucose | ARA_LEAF | p-PcUBI | GC | 115 | 341 |
| 55385 | mitochondrial | Sll1920 | g-1-p | ARA_LEAF | p-PcUBI | GC | 20 | 106 |
| 55385 | non-targ | Sll1920 | g-1-p | ARA_LEAF | p-PcUBI | GC | 20 | 136 |
| 55385 | mitochondrial | Sll1920 | my-inos | ARA_LEAF | p-PcUBI | GC | 25 | 28 |
| 55385 | non-targ | Sll1920 | raffinose | ARA_LEAF | p-PcUBI | LC | 85 | 216 |
| 55771 | non-targ | Slr0237 | g-1-p | ARA_LEAF | p-PcUBI | GC | 31 | 55 |
| 86540 | mitochondrial | Slr0239 | fructose | ARA_LEAF | p-PcUBI | GC | 76 | 426 |
| 86540 | mitochondrial | Slr0239 | glucose | ARA_LEAF | p-PcUBI | GC | 65 | 167 |
| 11990 | non-targ | Slr0338 | g-1-p | ARA_LEAF | p-PcUBI | GC | 18 | 64 |
| 11990 | non-targ | Slr0338 | THP | ARA_LEAF | p-PcUBI | GC | 43 | 133 |
| 56514 | non-targ | Slr0600 | g-1-p | ARA_LEAF | p-PcUBI | GC | 18 | 91 |
| 56894 | mitochondrial | Slr0710 | g-1-p | ARA_LEAF | p-PcUBI | GC | 24 | 37 |
| 87397 | plastidic | Slr0721 | g-1-p | ARA_LEAF | p-PcUBI | GC | 24 | 119 |
| 57235 | plastidic | Slr0739 | sucrose | ARA_LEAF | p-PcUBI | GC | 26 | 34 |
| 147346 | mitochondrial | Slr0757 | fructose | ARA_LEAF | p-PcUBI | GC | 100 | 243 |
| 147346 | mitochondrial | Slr0757 | glucose | ARA_LEAF | p-PcUBI | GC | 72 | 114 |
| 147346 | mitochondrial | Slr0757 | g-1-p | ARA_LEAF | p-PcUBI | GC | 39 | 56 |
| 57679 | mitochondrial | Slr0782 | g-1-p | ARA_LEAF | p-PcUBI | GC | 33 | 68 |
| 87610 | mitochondrial | Slr0862 | fructose | ARA_LEAF | p-PcUBI | GC | 80 | 98 |
| 87610 | mitochondrial | Slr0862 | glucose | ARA_LEAF | p-PcUBI | GC | 54 | 88 |
| 147407 | mitochondrial | Slr0905 | g-1-p | ARA_LEAF | p-PcUBI | GC | 32 | 57 |
| 119222 | mitochondrial | Slr0949 | my-inos | ARA_LEAF | p-PcUBI | GC | 25 | 56 |
| 57734 | non-targ | Slr1096 | g-1-p | ARA_LEAF | p-PcUBI | GC | 31 | 90 |
| 57734 | non-targ | Slr1096 | raffinose | ARA_LEAF | p-PcUBI | LC | 81 | 216 |
| 101776 | plastidic | Slr1107 | my-inos | ARA_LEAF | p-PcUBI | GC | 21 | 26 |
| 116713 | mitochondrial | Slr1350 | g-1-p | ARA_LEAF | p-PcUBI | GC | 26 | 61 |
| 58472 | mitochondrial | Slr1369 | g-1-p | ARA_LEAF | p-PcUBI | GC | 39 | 63 |
| 58472 | mitochondrial | Slr1369 | my-inos | ARA_LEAF | p-PcUBI | GC | 29 | 62 |
| 58668 | mitochondrial | Slr1492 | fructose | ARA_LEAF | p-PcUBI | GC | 82 | 150 |
| 12070 | mitochondrial | Slr1655 | sucrose | ARA_LEAF | p-PcUBI | GC | 26 | 64 |
| 58731 | plastidic | Slr1739 | fructose | ARA_LEAF | p-PcUBI | GC | 84 | 407 |
| 58751 | plastidic | Slr1742 | fructose | ARA_LEAF | p-PcUBI | GC | 76 | 91 |
| 58751 | plastidic | Slr1742 | my-inos | ARA_LEAF | p-PcUBI | GC | 27 | 35 |
| 147457 | plastidic | Slr1743 | g-1-p | ARA_LEAF | p-PcUBI | GC | 28 | 51 |
| 147637 | plastidic | Slr1779 | raffinose | ARA_LEAF | p-PcUBI | LC | 55 | 176 |
| 12140 | plastidic | Slr1791 | g-1-p | ARA_LEAF | p-PcUBI | GC | 21 | 48 |
| 12341 | plastidic | Slr2072 | g-1-p | ARA_LEAF | p-PcUBI | GC | 28 | 82 |
| 12341 | plastidic | Slr2072 | THP | ARA_LEAF | p-PcUBI | GC | 34 | 121 |
| 59370 | plastidic | Slr2124 | fructose | ARA_LEAF | p-PcUBI | GC | 152 | 214 |
| 59370 | plastidic | Slr2124 | glucose | ARA_LEAF | p-PcUBI | GC | 123 | 150 |
| 59370 | plastidic | Slr2124 | g-1-p | ARA_LEAF | p-PcUBI | GC | 31 | 31 |
| 59370 | plastidic | Slr2124 | my-inos | ARA_LEAF | p-PcUBI | GC | 24 | 38 |
| 59370 | plastidic | Slr2124 | sucrose | ARA_LEAF | p-PcUBI | GC | 24 | 45 |
| 147836 | mitochondrial | Slr2132 | raffinose | ARA_LEAF | p-PcUBI | LC | 75 | 205 |
| 136907 | plastidic | Smr0004 | g-1-p | ARA_LEAF | p-PcUBI | GC | 31 | 52 |
| 136907 | plastidic | Smr0004 | raffinose | ARA_LEAF | p-PcUBI | LC | 79 | 166 |
| 136907 | plastidic | Smr0004 | THP | ARA_LEAF | p-PcUBI | GC | 33 | 78 |
| 12698 | non-targ | TTC0019 | fructose | ARA_LEAF | p-PcUBI | GC | 83 | 4740 |
| 12698 | non-targ | TTC0019 | glucose | ARA_LEAF | p-PcUBI | GC | 62 | 1517 |
| 12698 | non-targ | TTC0019 | sh-7-p | ARA_LEAF | p-PcUBI | GC | 57 | 296 |
| 60301 | non-targ | TTC0035 | fructose | ARA_LEAF | p-PcUBI | GC | 77 | 154 |
| 60301 | non-targ | TTC0035 | glucose | ARA_LEAF | p-PcUBI | GC | 51 | 84 |
| 60301 | non-targ | TTC0035 | sucrose | ARA_LEAF | p-PcUBI | GC | 27 | 34 |
| 60859 | non-targ | TTC0216 | g-1-p | ARA_LEAF | p-PcUBI | GC | 32 | 117 |
| 60859 | non-targ | TTC0216 | THP | ARA_LEAF | p-PcUBI | GC | 46 | 95 |
| 147944 | non-targ | TTC0865 | my-inos | ARA_LEAF | p-PcUBI | GC | 30 | 49 |
| 61723 | non-targ | TTC1193 | fructose | ARA_LEAF | p-PcUBI | GC | 88 | 1786 |
| 61723 | non-targ | TTC1193 | glucose | ARA_LEAF | p-PcUBI | GC | 63 | 1072 |
| 61723 | non-targ | TTC1193 | g-1-p | ARA_LEAF | p-PcUBI | GC | 38 | 77 |
| 62079 | non-targ | TTC1386 | fructose | ARA_LEAF | p-PcUBI | GC | 363 | 1435 |
| 62079 | non-targ | TTC1386 | glucose | ARA_LEAF | p-PcUBI | GC | 197 | 296 |
| 12974 | non-targ | TTC1550 | fructose | ARA_LEAF | p-PcUBI | GC | 128 | 915 |
| 12974 | non-targ | TTC1550 | g-1-p | ARA_LEAF | p-PcUBI | GC | 19 | 53 |
| 12974 | non-targ | TTC1550 | sucrose | ARA_LEAF | p-PcUBI | GC | 24 | 83 |
| 148559 | non-targ | TTC1893 | raffinose | ARA_LEAF | p-PcUBI | LC | 66 | 72 |
| 148559 | non-targ | TTC1893 | THP | ARA_LEAF | p-PcUBI | GC | 47 | 62 |
| 62160 | non-targ | TTC1918 | g-1-p | ARA_LEAF | p-PcUBI | GC | 21 | 42 |
| 62244 | non-targ | XM_473199 | g-1-p | ARA_LEAF | p-PcUBI | GC | 18 | 54 |
| 62524 | non-targ | Ybl021c | my-inos | ARA_LEAF | Big35S | GC | 26 | 84 |
| 62524 | non-targ | Ybl021c | sucrose | ARA_LEAF | Big35S | GC | 25 | 53 |
| 88879 | plastidic | Ybr249c | g-1-p | ARA_LEAF | p-Super | GC | 32 | 59 |
| 89317 | non-targ | Ybr296c | g-1-p | ARA_LEAF | p-PcUBI | GC | 35 | 46 |
| 89317 | non-targ | Ybr296c | THP | ARA_LEAF | p-PcUBI | GC | 37 | 53 |
| 96980 | plastidic | Ycl027w | fructose | ARA_LEAF | p-Super | GC | 61 | 266 |
| 96980 | plastidic | Ycl027w | g-1-p | ARA_LEAF | p-Super | GC | 35 | 66 |
| 89395 | plastidic | Ycl064c | my-inos | ARA_LEAF | p-Super | GC | 28 | 48 |
| 89395 | plastidic | Ycl064c | sucrose | ARA_LEAF | p-Super | GC | 37 | 37 |
| 108113 | non-targ | Ycr012w | fructose | ARA_LEAF | Big35S | GC | 83 | 188 |
| 108113 | non-targ | Ycr012w | glucose | ARA_LEAF | Big35S | GC | 81 | 181 |
| 148579 | non-targ | Ycr047w-a | raffinose | ARA_LEAF | Big35S | LC | 69 | 234 |
| 63264 | plastidic | Ycr102c | THP | ARA_LEAF | p-Super | GC | 39 | 87 |
| 122228 | non-targ | Ydl066w | galactinol | ARA_SEED_2 | p-PcUBI | GC | 37 | 61 |
| 120045 | non-targ | Ydl126c | my-inos | ARA_LEAF | Big35S | GC | 24 | 35 |
| 14171 | non-targ | Ydl155w | my-inos | ARA_LEAF | Big35S | GC | 33 | 112 |
| 137498 | non-targ | Ydl159w-a | raffinose | ARA_LEAF | Big35S | LC | 57 | 204 |
| 14302 | plastidic | Ydl168w | g-1-p | ARA_LEAF | p-Super | GC | 34 | 126 |
| 89483 | plastidic | Ydl188c | g-1-p | ARA_LEAF | p-Super | GC | 40 | 58 |
| 148583 | plastidic | YDL222C | THP | ARA_LEAF | p-Super | GC | 36 | 79 |
| 113313 | plastidic | Ydl235c | g-1-p | ARA_LEAF | p-Super | GC | 31 | 88 |
| 113313 | plastidic | Ydl235c | my-inos | ARA_LEAF | p-Super | GC | 30 | 103 |
| 113313 | plastidic | Ydl235c | THP | ARA_LEAF | p-Super | GC | 48 | 88 |
| 63334 | plastidic | Ydr044w | sucrose | ARA_LEAF | p-PcUBI | GC | 31 | 77 |
| 63544 | plastidic | Ydr046c | glucose | ARA_LEAF | p-Super | GC | 36 | 43 |
| 90103 | plastidic | Ydr183w | fructose | ARA_LEAF | p-Super | GC | 158 | 249 |
| 90103 | plastidic | Ydr183w | glucose | ARA_LEAF | p-Super | GC | 60 | 74 |
| 90165 | non-targ | Ydr507c | g-1-p | ARA_LEAF | Big35S | GC | 18 | 88 |
| 148614 | non-targ | Yel011w | fructose | ARA_LEAF | Big35S | GC | 113 | 539 |
| 148614 | non-targ | Yel011w | glucose | ARA_LEAF | Big35S | GC | 71 | 431 |
| 97294 | non-targ | Yel030c-a | THP | ARA_LEAF | Big35S | GC | 33 | 87 |
| 128511 | non-targ | Yel046c | g-1-p | ARA_LEAF | Big35S | GC | 21 | 112 |
| 148811 | plastidic | Yer024w | glucose | ARA_LEAF | p-Super | GC | 47 | 92 |
| 148833 | plastidic | Yer177w | raffinose | ARA_LEAF | p-PcUBI | LC | 112 | 148 |
| 64144 | non-targ | Yfl019c | my-inos | ARA_LEAF | Big35S | GC | 28 | 275 |
| 64144 | non-targ | Yfl019c | sucrose | ARA_LEAF | Big35S | GC | 26 | 126 |
| 149219 | non-targ | Yfr053c | sucrose | ARA_LEAF | Big35S | GC | 27 | 45 |
| 120436 | plastidic | Ygl039w | g-1-p | ARA_LEAF | p-PcUBI | GC | 30 | 57 |
| 120436 | plastidic | Ygl039w | THP | ARA_LEAF | p-PcUBI | GC | 36 | 92 |
| 90278 | plastidic | Ygl096w | g-1-p | ARA_LEAF | p-Super | GC | 18 | 48 |
| 90278 | plastidic | Ygl096w | THP | ARA_LEAF | p-Super | GC | 33 | 86 |
| 64177 | non-targ | Ygl237c | raffinose | ARA_LEAF | Big35S | LC | 68 | 290 |
| 130742 | plastidic | Ygl256w | g-1-p | ARA_LEAF | p-Super | GC | 44 | 105 |
| 130742 | plastidic | Ygl256w | THP | ARA_LEAF | p-Super | GC | 40 | 101 |
| 14715 | plastidic | Yhl013c | glucose | ARA_LEAF | p-Super | GC | 137 | 173 |
| 14715 | plastidic | Yhl013c | g-1-p | ARA_LEAF | p-Super | GC | 18 | 38 |
| 64563 | plastidic | Yil074c | g-1-p | ARA_LEAF | p-Super | GC | 36 | 70 |
| 149454 | plastidic | Yil083c | fructose | ARA_LEAF | p-Super | GC | 89 | 140 |
| 64964 | plastidic | Yjl073w | my-inos | ARA_LEAF | p-Super | GC | 25 | 34 |
| 97631 | non-targ | Yjl127w-a | THP | ARA_LEAF | Big35S | GC | 39 | 102 |
| 149632 | non-targ | Yjl153c | fructose | ARA_SEED_2 | p-PcUBI | GC | 51 | 79 |
| 149632 | non-targ | Yjl153c | galactitol | ARA_SEED_2 | p-PcUBI | GC | 96 | 292 |
| 149632 | non-targ | Yjl153c | my-inos | ARA_SEED_2 | p-PcUBI | GC | 24 | 70 |
| 97635 | non-targ | Yjl219w | sorbitol | ARA_SEED_2 | p-PcUBI | GC | 121 | 350 |
| 149801 | non-targ | Yjr066w | my-inos | ARA_LEAF | Big35S | GC | 26 | 87 |
| 14843 | non-targ | Yjr130c | glucose | ARA_LEAF | Big35S | GC | 45 | 92 |
| 149868 | non-targ | Ykl060c | fructose | ARA_LEAF | p-PcUBI | GC | 82 | 133 |
| 149868 | non-targ | Ykl060c | sh-7-p | ARA_LEAF | p-PcUBI | GC | 43 | 61 |
| 150063 | plastidic | Ykl192c | g-1-p | ARA_LEAF | p-PcUBI | GC | 42 | 49 |
| 130903 | non-targ | Ykr072c | sucrose | ARA_LEAF | Big35S | GC | 23 | 51 |
| 98075 | non-targ | Ykr097w | glucose | ARA_LEAF | p-PcUBI | GC | 62 | 110 |
| 98075 | plastidic | Ykr097w | glucose | ARA_LEAF | p-Super | GC | 70 | 93 |
| 150603 | non-targ | Ylr025w | sucrose | ARA_LEAF | Big35S | GC | 31 | 31 |
| 91045 | plastidic | Ylr043c | g-1-p | ARA_LEAF | p-Super | GC | 19 | 78 |
| 150682 | non-targ | Ylr081w | fructose | ARA_LEAF | p-PcUBI | GC | 68 | 128 |
| 91973 | plastidic | Ymr120c | verbascose | ARA_LEAF | p-Super | LC | 135 | 3405 |
| 130986 | plastidic | Ymr324c | g-1-p | ARA_LEAF | p-Super | GC | 34 | 58 |
| 117495 | non-targ | Ynl064c | my-inos | ARA_LEAF | Big35S | GC | 28 | 50 |
| 117495 | non-targ | Ynl064c | sucrose | ARA_LEAF | Big35S | GC | 25 | 31 |
| 67646 | plastidic | Ynr019w | raffinose | ARA_LEAF | p-PcUBI | LC | 64 | 132 |
| 15179 | plastidic | Yol160w | my-inos | ARA_LEAF | p-Super | GC | 27 | 30 |
| 151862 | plastidic | Yor163w | glucose | ARA_LEAF | p-Super | GC | 42 | 80 |
| 92540 | plastidic | Yor244w | g-1-p | ARA_LEAF | p-Super | GC | 31 | 53 |
| 151910 | plastidic | Ypl138c | THP | ARA_LEAF | p-Super | GC | 32 | 115 |
| 151927 | plastidic | Ypl144w | g-1-p | ARA_LEAF | p-Super | GC | 33 | 71 |
| 151927 | plastidic | Ypl144w | THP | ARA_LEAF | p-Super | GC | 36 | 66 |
| 151931 | plastidic | Ypr098c | g-1-p | ARA_LEAF | p-Super | GC | 47 | 110 |
| 151931 | plastidic | Ypr098c | THP | ARA_LEAF | p-Super | GC | 63 | 122 |
| 151958 | plastidic | Ypr156c | g-1-p | ARA_LEAF | p-PcUBI | GC | 19 | 58 |
| 151958 | plastidic | Ypr156c | my-inos | ARA_LEAF | p-PcUBI | GC | 27 | 92 |
| 68413 | non-targ | Zm_4842_BE510522 | fructose | ARA_LEAF | p-PcUBI | GC | 106 | 493 |
| 68413 | non-targ | Zm_4842_BE510522 | glucose | ARA_LEAF | p-PcUBI | GC | 68 | 257 |
| 140626 | plastidic | 44849766_CANOLA | my-inos | ARA_LEAF | p-PcUBI | GC | 36 | 104 |
| 15187 | non-targ | 47266012_SOYBEAN | fructose | ARA_LEAF | p-PcUBI | GC | 78 | 200 |
| 15187 | non-targ | 47266012_SOYBEAN | glucose | ARA_LEAF | p-PcUBI | GC | 60 | 140 |
| 15187 | non-targ | 47266012_SOYBEAN | g-1-p | ARA_LEAF | p-PcUBI | GC | 21 | 69 |
| 15187 | non-targ | 47266012_SOYBEAN | my-inos | ARA_LEAF | p-PcUBI | GC | 27 | 62 |
| 15187 | non-targ | 47266012_SOYBEAN | sh-7-p | ARA_LEAF | p-PcUBI | GC | 48 | 72 |
| 69 | non-targ | 49747384_SOYBEAN | glucose | ARA_LEAF | p-PcUBI | GC | 101 | 120 |
| 69 | non-targ | 49747384_SOYBEAN | g-1-p | ARA_LEAF | p-PcUBI | GC | 27 | 72 |
| 69 | non-targ | 49747384_SOYBEAN | my-inos | ARA_LEAF | p-PcUBI | GC | 33 | 71 |
| 69 | non-targ | 49747384_SOYBEAN | raffinose | ARA_LEAF | p-PcUBI | LC | 64 | 155 |
| 69 | non-targ | 49747384_SOYBEAN | sh-7-p | ARA_LEAF | p-PcUBI | GC | 46 | 85 |
| 16883 | plastidic | 59582753_SOYBEAN | fructose | ARA_LEAF | p-PcUBI | GC | 77 | 404 |
| 16883 | plastidic | 59582753_SOYBEAN | glucose | ARA_LEAF | p-PcUBI | GC | 98 | 367 |
| 16883 | plastidic | 59582753_SOYBEAN | g-1-p | ARA_LEAF | p-PcUBI | GC | 32 | 101 |
| 16883 | plastidic | 59582753_SOYBEAN | sh-7-p | ARA_LEAF | p-PcUBI | GC | 56 | 350 |
| 16883 | plastidic | 59582753_SOYBEAN | sucrose | ARA_LEAF | p-PcUBI | GC | 30 | 79 |
| 16883 | plastidic | 59582753_SOYBEAN | THP | ARA_LEAF | p-PcUBI | GC | 37 | 139 |
| 149528 | non-targ | YJL072C | fructose | ARA_LEAF | Big35S | GC | 1 | 418 |
| 150960 | plastidic | YLR153C | glucose | ARA_LEAF | p-Super | GC | 12 | 372 |

Column 1 shows the SEQ ID NO, Column 2 shows the expression type (targeted or non-targeted), Column 3 shows the “gene name” (locus), Column 4 shows the metabolite analyzed, Column 5 indicates the A. thaliana source tissue analyzed, Column 6 indicates the used promoter for expression, Column 7 indicates the analytical method. Columns 8 and 9 show the minimum and the maximum increase of the analyzed metabolite (in percent) in comparison to the wild type (ratio_by _WT, given as percent increase).

The term “non-targ” in Table d, Column 2 which shows the expression type means “non-targeted”.

The term “THP” in Table d, Column 4 which shows the metabolite analyzed means “3,4,5-trihydroxypentanoic acid”.

The term “sh-7-p” in Table d, Column 4 which shows the metabolite analyzed means “sedoheptulose-7-phosphate”.

The term “g-1-p” in Table d, Column 4 which shows the metabolite analyzed means “glucose-1-phosphate”.

The term “my-inos” in Table d, Column 4 which shows the metabolite analyzed means “myo-inositol”.

The term “sacch a” in Table d, Column 4 which shows the metabolite analyzed means “saccharic acid”.

When the analyses were repeated independently, all results proved to be significant.

[0505.1.7.17] to [0515.1.7.17] for the disclosure of these paragraphs see [0505.1.7.7] to [0515.1.7.7] above

In a further embodiment, the present invention relates in paragraphs [0000.1.7.18.] to [0514.1.7.18.] to a further process for the production of the fine chemical cerotic acid, heneicosanoic acid, lignoceric acid, melissic acid and/or montanic acid as defined below and corresponding embodiments as described herein as follows.

for the disclosure of this paragraph see [0001.1.7.7] above

Living organisms synthesize many different fatty acids. Most of them are incorporated into complex lipids, which constitute the major structural component of membranes and also the major storage product in plants and animals. Some of these fatty acids are the so called very-long-chain fatty acids (VLCFAs) which are composed of 20 or more carbons in length (i.e. >C18). VLCFAs are involved in many different physiological functions in different organisms.

In plants VLCFAs are essential for example in plant development and necessary for the synthesis of seed storage triacylglycerols, epicuticular waxes, and sphingolipids.

In plants, fatty acids up to C18-chains are formed in the plastid (fatty acid de novo synthesis) while those with C>18 are synthesized by so called elongases in the endoplasmic reticulum of the cytosol. The first reaction of elongation involves condensation of malonyl-CoA with a long chain substrate, a CoA-activated acyl substrates (stemming from fatty acid de novo synthesis or previous rounds of elongation) producing a β-ketoacyl-CoA, yielding elongation with a C2-unit. Subsequent reactions are reduction of β-hydroxyacyl-CoA, dehydration to an enoyl-CoA, followed by a second reduction to form the elongated acyl-CoA. The β-ketoacyl-CoA synthase (KCS) catalyzing the condensation reaction plays a key role in determining the chain length of fatty acid products found in seed oils and is the rate-limiting enzyme for seed VLCFA production (Lassner et al., Plant Cell, 8(1996), 281-292). The elongation process can be repeated to yield members that are 20, 22, and 24 carbons long. Plants produce very long chain fatty acids such as heneicosanoic acid (C21:0), lignoceric acid (C24:0), cerotic acid (C26:0), montanic acid (C28:0) and/or melissic acid (C30:0).

Three different pathways then lead to the transformation of VLCFAs: in the epidermis, (i) through the “acylreduction pathway”, primary alcohols and wax-esters are formed by fatty acyl-coA reductases (FAR) and wax synthases (WS), respectively, (ii) in the “decarbonylation pathway”, VLCFAs are reduced to alkanes, which in stems are oxidised by mid-chain alkane hydroxylase (MAH 1) to form secondary alcohols and ketones (FIG. 1, reviewed in ref. 5). Finally, (iii) in all cells, VLCFAs and long chain bases (LCBs) (generated from C16 and C18 fatty acids) are the precursors for the synthesis of sphingolipids (Rafaele et al., Plant Signal Behay. (2009), 4(2), 94-99).

Very-long-chain fatty acids (VLCFAs), like are essential components for the vitality of higher plants. The key enzyme of VLCFA biosynthesis, the extraplastidary fatty acid elongase, is shown for to be the primary target site of chloroacetamide herbicides. With an analysis of the fatty acid composition and the metabolism of 140-labelled precursors (sterate, malonate, acetate), the reduction of VLCFAs was determined in vivo. The inhibition of the recombinant protein substantiates the first and rate-limiting step of VLCFA biosynthesis, the condensation of acyl-CoA with malonyl-CoA to β-ketoacyl-CoA, to be the primary target site of chloroacetamides (150=10-100 nM).

Cerotic acid (26:0) (hexacosanoic acid) is a component of beeswax. Echinacea angustifolia extracts are sold as natural health products comprising the very long chain fatty acid cerotic acid. Cerotic acid is used in cosmetics as a constituent in hairstyling products. Like most long chain fatty acids with an uneven number of carbons, heneicosanoic acid is found rarely and in rather low concentrations in nature. With more than 18 carbon atoms.

It has been reported that heneicosanoic acid is found in higher concentrations (up to 5% of total fatty acids) in some Basidiomycetes (Armillaria) (K. D. Cox; H. Scherm; M. B. Riley: Characterization of Armillaria spp. from peach orchards in the southeastern United States using fatty acid methyl ester profiling, Mycological Research 110, 414-422, 2006).

Because of it is rarely encountered in biological samples, heneicosanoic acid is commonly used as internal standard in gas chromatography of fatty acids.

Lignoceric acid (24:0) (tetracosanoic acid) is a component of rapeseed oil (up to 1%) and peanut oil (1-3%). It is further found in wood tar, various cerebrosides, and in small amounts in most natural fats. The fatty acids of peanut oil contain small amounts of lignoceric acid (1.1%-2.2%). This fatty acid is also a byproduct of lignin production. Reduction of lignoceric acid yields lignoceryl alcohol.

Montanic acid and it's esters with long chain alcohols (C-24, C-26 and C-28) are part of montan wax. Montan wax, also known as lignite wax or OP wax, is a hard wax obtained by solvent extraction of certain types of lignite or brown coal. Commercially viable deposits exist in only a few locations, including Amsdorf, Germany, and in the lone Basin near lone, California. Montan wax, composed of non-glyceride long-chain (C24-030) carboxylic acid esters (62-68 weight %), free long-chain organic acids (22-26%), long-chain alcohols, ketones, and hydrocarbons (7-15%) and resins, is in effect a fossilized plant wax.

The frequent used technical montanic acid is a mixture of saturated fatty acids with 24 tp 32 carbons, Esters of montanic acid are known as food additive “E912”. E912 is used to treat the outer surface of citrus fruits.

Melissic acid (C30:0) (triacontanoic acid) is a component of beeswax.

Beeswax (cera alba) is obtained from the product excreted by certain glands of the honeybee from which the honeycomb is made. It is freed of solid impurities by melting and centrifugation (cera flava). Finally, it is bleached completely white (cera alba). Beeswax consists of 10-15 percent paraffin carbohydrates, 35-37-percent esters of C16 to C36 fatty acids and about 15 percent cerotic acid, melissic acid and their homologues. Beeswax is used as a thickener and a humectant in the manufacture of ointments, creams, lipsticks and other cosmetics and skincare products as an emulsifier, emollient, moisturizer and film former.

Beeswax is also used for the production of candles.

Wax is a general term used to refer to the mixture of long-chain apolar lipids forming a protective coating (cutin in the cuticle) on plant leaves and fruits but also in animals (wax of honeybee, cuticular lipids of insects, spermaceti of the sperm whale, skin lipids, uropygial glands of birds, depot fat of planktonic crustacea), algae, fungi and bacteria.

Many of the waxes found in nature have commercial uses in the lubricant, food and cosmetic industry. Jojoba oil has long been suggested as a putative resource of wax, since this desert shrub is unusual in its capacity to produce waxes rather than triacylglycerols (TAG) as seed storage lipids. These waxes are esters of very-long-chain-fatty acids and fatty alcohols (Miwa, 1971, J Am Oil Chem Soc 48, 259-264). As the production cost for jojoba wax, which is primarily used for cosmetic applications, is high, there is a need to engineer crop plants to produce high level of wax esters in its seed oil.

Plant aerial surfaces are covered by epicuticular waxes, complex mixtures of very long (C20-034) fatty acids, alkanes, aldehydes, ketones and esters. In addition to repelling atmospheric water they prevent dessication and are therefore an important determinant of drought resistance (Riederer and Schreiber, 2001, J. Exp. Bot 52, 2023-2032). Beside abiotic stress resistance the wax layer is part of the plant defense against biotic stressor, especially insects as for example described by Marcell and Beattie, 2002, Mol Plant Microbe Interact. 15(12), 1236-44. Furthermore they provide stability to pollen grains, thus influencing fertility and productivity.

Increased wax production in transgenic plants has for example been reported by Broun et al., 2004, Proc Natl. Acad. Sci, 101, 4706-4711. The authors overexpressed the transcriptional activator WIN1 in Arabidopsis, leading to increased wax load on arial organs. As this resulted in a complex change in the wax profile and the transgenic overexpressors had characteristic alterations in growth and development (Broun et al., 2004, Proc Natl. Acad. Sci, 101, 4706-4711) there is still a need for a more controlled increased production of defined VLCFAs.

Very long chain fatty alcohols obtained from plant waxes and beeswax have also been reported to lower plasma cholesterol in humans and existing data support the hypothesis that VLCFA exert regulatory roles in cholesterol metabolism in the peroxisome and also alter LDL uptake and metabolism (discussed in Hargrove et al., 2004, Exp Biol Med (Maywood), 229(3): 215-26).

Due to these interesting physiological roles and the nutritional, cosmetic and agrobiotechnological potential of cerotic acid, heneicosanoic acid, lignoceric acid, melissic acid and/or montanic acid there is a need to identify the genes of enzymes and other proteins involved in VLCFA metabolism, and to generate mutants or transgenic plant lines with which to modify the cerotic acid, heneicosanoic acid, lignoceric acid, melissic acid and/or montanic acid content in plants.

Economical methods for producing waxes, VLCFAs or derivates thereof, life salts, ethers esters, oxo and/or hydroxy derivates and/or reduced derivates like alcohols and food- and feed-stuffs with increased cerotic acid, heneicosanoic acid, lignoceric acid, melissic acid and/or montanic acid content are therefore very important.

Particularly economical methods are biotechnological methods utilizing waxes and/or VLCFA-producing organisms which are either natural or optimized by genetic modification.

There is a constant need for providing novel enzyme activities or direct or indirect regulators and thus alternative methods with advantageous properties for producing waxes and/or VLCFA in organisms, e.g. in transgenic organisms.

The content of a some VLCFAs in a cell was also increased by increasing or decreasing certain activities as disclosed in WO2006/069610, WO 2007/087815 or WO 2008/034648.

Improving the quality of foodstuffs and animal feeds is an important task of the food-and-feed industry. Especially advantageous for the quality of foodstuffs and animal feeds is as balanced as possible a fatty acid, waxe and/or lipid profile since a great excess of some fatty acids, waxes and/or lipids above a specific concentration in the food has no further positive effect on the utilization of the food since other fatty acid, waxes and/or lipid suddenly become limiting. A further increase in quality is only possible via addition of further fatty acid, waxe and/or lipid, which are limiting under these conditions. The targeted addition of the limiting fatty acid, waxe and/or lipid in the form of synthetic products must be carried out with extreme caution in order to avoid fatty acid, waxe and/or lipid imbalance.

To ensure a high quality of foods and animal feeds, it is therefore necessary to add a plurality of fatty acids, waxes and/or lipids in a balanced manner to suit the respective organism. Accordingly, there is still a great demand for new and more suitable genes, which encode enzymes or regulators, which participate in the biosynthesis of fatty acid, waxes and/or lipid, in particular VLCFAs and make it possible to produce certain VLCFAs specifically on an industrial scale without unwanted byproducts being formed. In the selection of genes for biosynthesis or regulation two characteristics above all are particularly important. On the one hand, there is as ever a need for improved processes for obtaining the highest possible contents of fatty acids, waxes and/or lipids like cerotic acid, heneicosanoic acid, lignoceric acid, melissic acid and/or montanic acid and on the other hand as less as possible byproducts should be produced in the production process.

It is an object of the present invention to develop an inexpensive process for the synthesis of cerotic acid, heneicosanoic acid, lignoceric acid, melissic acid and/or montanic acid

It is a further object of the present invention to develop an inexpensive process for the synthesis of waxes, e.g. VFCLAs, in particular cerotic acid, heneicosanoic acid, lignoceric acid, melissic acid and/or montanic acid and to assure that the VFCLAs, in particular cerotic acid, heneicosanoic acid, lignoceric acid, melissic acid and/or montanic acid are more accessible and facilely to isolate and recover in an industrial scale from the producing organism, preferably from a plant.

for the disclosure of this paragraph see [0013.1.7.7] above

Accordingly, in a first embodiment, the invention relates to a process for the production of at least one fine chemical selected from the group consisting of: cerotic acid, heneicosanoic acid, lignoceric acid, melissic acid and montanic acid, or, in other words, of the “fine chemical” or “fine chemical of the invention”.

The terms “fine chemical of the invention”, “fine chemical” or “the fine chemical” are used herein equally and relate in context of the paragraphs or sections [0014.1.18.18] to [0514.1.7.18] essentially to the metabolite or the metabolites indicated in column 7, application no. 18 of Tables I, II and IV in the respective line.

Further, the term “in context of any of the paragraphs [0014.1.18.18] to [0514.1.7.18]” as used herein means that for any of said paragraphs [0014.1.18.18] to [0514.1.7.18] the term “the fine chemical” is understood to follow the definition of paragraphs or sections [0014.1.18.18] and [0015.1.18.18], independently whether it refers to any other paragraph or not and whether the reference recites the term “fine chemical” in an other context.

Thus, in cases where one or more paragraphs or sections are incorporated by reference into any of the present paragraphs [0014.1.18.18] to [0514.1.7.18], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to the definition of paragraph [0014.1.18.18] and [0015.1.18.18].

Accordingly, in one embodiment, the term “fine chemical of the invention”, “fine chemical” or “the fine chemical” as used herein (alone or in combination, like in FCRP) means “cerotic acid” in context of the nucleic acid or polypeptide sequences listed in the respective same line of any one of Tables I to IV of application no. 18 and indicating in column 7 the metabolite “cerotic acid”.

In one embodiment, the term cerotic acid or the term “fine chemical” mean in context of the paragraphs or sections [0014.1.18.18] to [0514.1.7.18] at least one chemical compound with an activity of the above mentioned cerotic acid, respectively.

Accordingly, in one embodiment, the term “fine chemical of the invention”, “fine chemical” or “the fine chemical” as used herein (alone or in combination, like in FCRP) means “heneicosanoic acid” in context of the nucleic acid or polypeptide sequences listed in the respective same line of any one of Tables I to IV of application no. 18 and indicating in column 7 the metabolite “heneicosanoic acid”.

In one embodiment, the term heneicosanoic acid or the term “fine chemical” mean in context of the paragraphs or sections [0014.1.18.18] to [0514.1.7.18] at least one chemical compound with an activity of the above mentioned heneicosanoic acid, respectively.

Accordingly, in one embodiment, the term “fine chemical of the invention”, “fine chemical” or “the fine chemical” as used herein (alone or in combination, like in FCRP) means “lignoceric acid” in context of the nucleic acid or polypeptide sequences listed in the respective same line of any one of Tables I to IV of application no. 18 and indicating in column 7 the metabolite “lignoceric acid”.

In one embodiment, the term lignoceric acid or the term “fine chemical” mean in context of the paragraphs or sections [0014.1.18.18] to [0514.1.7.18] at least one chemical compound with an activity of the above mentioned lignoceric acid, respectively.

Accordingly, in one embodiment, the term “fine chemical of the invention”, “fine chemical” or “the fine chemical” as used herein (alone or in combination, like in FCRP) means “melissic acid” in context of the nucleic acid or polypeptide sequences listed in the respective same line of any one of Tables I to IV of application no. 18 and indicating in column 7 the metabolite “melissic acid”.

In one embodiment, the term melissic acid or the term “fine chemical” mean in context of the paragraphs or sections [0014.1.18.18] to [0514.1.7.18] at least one chemical compound with an activity of the above mentioned melissic acid respectively.

Accordingly, in one embodiment, the term “fine chemical of the invention”, “fine chemical” or “the fine chemical” as used herein (alone or in combination, like in FCRP) means “montanic acid” in context of the nucleic acid or polypeptide sequences listed in the respective same line of any one of Tables I to IV of application no. 18 and indicating in column 7 the metabolite “montanic acid”.

In one embodiment, the term montanic acid or the term “fine chemical” mean in context of the paragraphs or sections [0014.1.18.18] to [0514.1.7.18] at least one chemical compound with an activity of the above mentioned montanic acid, respectively.

Accordingly, in one embodiment, the terms “fine chemical of the invention”, “fine chemical” or “the fine chemical” mean in context of any of the paragraphs [0014.1.18.18] to [0514.1.7.18] cerotic acid, or derivates thereof, life salts, ethers esters, oxo and/or hydroxy derivates and/or reduced derivates like alcohols in free form or bound form. In a preferred embodiment, the term “the fine chemical” means cerotic acid or its salts, ester or ether, in free form or bound form.

In a preferred embodiment “cerotic acid” means cerotic acid in free form.

Accordingly, in one embodiment, the terms “fine chemical of the invention”, “fine chemical” or “the fine chemical” mean in context of any of the paragraphs [0014.1.18.18] to [0514.1.7.18] heneicosanoic acid, or derivates thereof, life salts, ethers esters, oxo and/or hydroxy derivates and/or reduced derivates like alcohols in free form or bound form. In a preferred embodiment, the term “the fine chemical” means heneicosanoic acid or its salts, ester or ether, in free form or bound form.

In a preferred embodiment “heneicosanoic acid” means heneicosanoic acid in free form.

Accordingly, in one embodiment, the terms “fine chemical of the invention”, “fine chemical” or “the fine chemical” mean in context of any of the paragraphs [0014.1.18.18] to [0514.1.7.18] lignoceric acid, or derivates thereof, life salts, ethers esters, oxo and/or hydroxy derivates and/or reduced derivates like alcohols in free form or bound form. In a preferred embodiment, the term “the fine chemical” means lignoceric acid or its salts, ester or ether, in free form or bound form.

In a preferred embodiment “lignoceric acid” means lignoceric acid in free form.

Accordingly, in one embodiment, the terms “fine chemical of the invention”, “fine chemical” or “the fine chemical” mean in context of any of the paragraphs [0014.1.18.18] to [0514.1.7.18] melissic acid or derivates thereof, life salts, ethers esters, oxo and/or hydroxy derivates and/or reduced derivates like alcohols in free form or bound form. In a preferred embodiment, the term “the fine chemical” means melissic acid or its salts, ester or ether, in free form or bound form.

In a preferred embodiment “melissic acid” means melissic acid in free form.

Accordingly, in one embodiment, the terms “fine chemical of the invention”, “fine chemical” or “the fine chemical” mean in context of any of the paragraphs [0014.1.18.18] to [0514.1.7.18] montanic acid, or derivates thereof, life salts, ethers esters, oxo and/or hydroxy derivates and/or reduced derivates like alcohols in free form or bound form. In a preferred embodiment, the term “the fine chemical” means montanic acid or its salts, ester or ether, in free form or bound form.

In a preferred embodiment “montanic acid” means montanic acid in free form.

Further, the term “in context of any of the paragraphs [0014.1.18.18] to [0514.1.7.18]” as used herein means that for any of said paragraphs [0014.1.18.18] to [0514.1.7.18] the term “the fine chemical” is understood to follow the definition of section [0014.1.18.18] or section [0015.1.18.18], independently whether it refers to any other paragraph or not and whether the reference recites the term “fine chemical” in an other context.

Thus, in cases where one or more paragraphs or sections are incorporated by reference into any of the present paragraphs [0014.1.18.18] to [0514.1.7.18], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to this definition of this paragraph [0015.1.18.18].

Further, the term “fine chemicals” as used herein relates to compositions comprising said fine chemical(s), i.e. comprising cerotic acid, heneicosanoic acid, lignoceric acid, melissic acid and/or montanic acid, respectively.

Accordingly, the present invention relates to a process for the production of lignoceric acid, which comprises
(a) increasing or generating one or more activities selected from the group consisting of eukaryotic translation initiation factor 5 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of thioredoxin family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of monodehydroascorbate reductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of glycosyl transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of gibberellin 20-oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of zinc finger protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of aminotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of glutamate-ammonia-ligase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of chaperone protein CIpB in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of purine nucleoside phosphorylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of threonine synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of serine protease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of ATP-binding component of a transport system in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of isochorismate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of leucyl/phenylalanyl-tRNA-protein transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of major facilitator superfamily transporter protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of cerotic acid, which comprises
(a) increasing or generating one or more activities selected from the group consisting of multidrug resistance protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerotic acid or a composition comprising cerotic acid in said non-human organism or in the culture medium surrounding said non-human organism.

Accordingly, the present invention relates to a process for the production of lignoceric acid, which comprises
(a) increasing or generating one or more activities selected from the group consisting of sodium/proton antiporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of cardiolipin synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of b1522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of cerotic acid, which comprises
(a) increasing or generating one or more activities selected from the group consisting of acid shock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerotic acid or a composition comprising cerotic acid in said non-human organism or in the culture medium surrounding said non-human organism.

Accordingly, the present invention relates to a process for the production of lignoceric acid, which comprises
(a) increasing or generating one or more activities selected from the group consisting of acid shock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of O-antigen chain length determinant in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of NADH dehydrogenase subunit N in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of 2,3-dihydroxy-2,3-dihydrophenylpropionatedehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of b3083-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of b3812-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of b4029-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of sll0064-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of agmatinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of uroporphyrin-III C-methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of binding-protein-dependent transport systems inner membrane component in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of malate dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of ABC metal ion transporter substrate-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of acyl transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of slr0383-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of geranylgeranyl pyrophosphate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of tryptophan synthase alpha chain in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of rubredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of Sec-independent protein translocase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of TTC0768-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of TTC1386-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of acetyl-coenzyme A synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of peroxisome assembly protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of heneicosanoic acid, which comprises
(a) increasing or generating one or more activities selected from the group consisting of inositol-3-phosphate synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of heneicosanoic acid or a composition comprising heneicosanoic acid in said non-human organism or in the culture medium surrounding said non-human organism.

Accordingly, the present invention relates to a process for the production of lignoceric acid, which comprises
(a) increasing or generating one or more activities selected from the group consisting of cystathionine gamma-synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of potassium transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of ylr065c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of yml093w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of mitochondrial inner membrane protease subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating one or more activities selected from the group consisting of mevalonate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of melissic acid which comprises
(a) increasing or generating one or more activities selected from the group consisting of yer152c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of melissic acid or a composition comprising melissic acid in said non-human organism or in the culture medium surrounding said non-human organism.

Accordingly, the present invention relates to a process for the production of montanic acid, which comprises
(a) increasing or generating one or more activities selected from the group consisting of yen 52c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of montanic acid or a composition comprising montanic acid in said non-human organism or in the culture medium surrounding said non-human organism.

Accordingly, the present invention relates to a process for the production of melissic acid which comprises
(a) increasing or generating one or more activities selected from the group consisting of b3644-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of melissic acid or a composition comprising melissic acid in said non-human organism or in the culture medium surrounding said non-human organism.

Accordingly, the present invention relates to a process for the production of montanic acid, which comprises
(a) increasing or generating one or more activities selected from the group consisting of b3644-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of montanic acid or a composition comprising montanic acid in said non-human organism or in the culture medium surrounding said non-human organism.

An embodiment of the present invention relates to a process for the production of cerotic acid, which comprises
(a1) increasing or generating the activity of a polypeptide comprising a polypeptide as depicted in a respective line in column 5 or 8 of Table II, application no. 18, or a homolog or a fragment thereof, a consensus sequence or at least one polypeptide motif as depicted in the respective line in column 8 of Table IV, application no. 18, whereby the respective line disclose in column 7 the fine chemical cerotic acid; or
(a2) increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, whereby the respective line disclose in column 7 the fine chemical cerotic acid; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of cerotic acid or a composition comprising cerotic acid in said non-human organism or in the culture medium surrounding said non-human organism.

An embodiment of the present invention relates to a process for the production of heneicosanoic acid, which comprises
(a1) increasing or generating the activity of a polypeptide comprising a polypeptide as depicted in a respective line in column 5 or 8 of Table II, application no. 18, or a homolog or a fragment thereof, a consensus sequence or at least one polypeptide motif as depicted in the respective line in column 8 of Table IV, application no. 18, whereby the respective line disclose in column 7 the fine chemical heneicosanoic acid; or
(a2) increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, whereby the respective line disclose in column 7 the fine chemical heneicosanoic acid; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of heneicosanoic acid or a composition comprising heneicosanoic acid in said non-human organism or in the culture medium surrounding said non-human organism.

An embodiment of the present invention relates to a process for the production of lignoceric acid, which comprises
(a1) increasing or generating the activity of a polypeptide comprising a polypeptide as depicted in a respective line in column 5 or 8 of Table II, application no. 18, or a homolog or a fragment thereof, a consensus sequence or at least one polypeptide motif as depicted in the respective line in column 8 of Table IV, application no. 18, whereby the respective line disclose in column 7 the fine chemical lignoceric acid; or
(a2) increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, whereby the respective line disclose in column 7 the fine chemical lignoceric acid; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of lignoceric acid or a composition comprising lignoceric acid in said non-human organism or in the culture medium surrounding said non-human organism.

An embodiment of the present invention relates to a process for the production of melissic acid which comprises
(a1) increasing or generating the activity of a polypeptide comprising a polypeptide as depicted in a respective line in column 5 or 8 of Table II, application no. 18, or a homolog or a fragment thereof, a consensus sequence or at least one polypeptide motif as depicted in the respective line in column 8 of Table IV, application no. 18, whereby the respective line disclose in column 7 the fine chemical melissic acid; or
(a2) increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, whereby the respective line disclose in column 7 the fine chemical melissic acid; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of melissic acid or a composition comprising melissic acid in said non-human organism or in the culture medium surrounding said non-human organism.

An embodiment of the present invention relates to a process for the production of montanic acid, which comprises
(a1) increasing or generating the activity of a polypeptide comprising a polypeptide as depicted in a respective line in column 5 or 8 of Table II, application no. 18, or a homolog or a fragment thereof, a consensus sequence or at least one polypeptide motif as depicted in the respective line in column 8 of Table IV, application no. 18, whereby the respective line disclose in column 7 the fine chemical montanic acid; or
(a2) increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, whereby the respective line disclose in column 7 the fine chemical montanic acid; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of montanic acid or a composition comprising montanic acid in said non-human organism or in the culture medium surrounding said non-human organism.

Accordingly, the increase or generation of one or more said activities is for example conferred by one or more expression products of said nucleic acid molecule, e.g. proteins. Accordingly, in the present invention described above, the increase or generation of one or more said activities is for example conferred by one or more protein(s) each comprising a polypeptide selected from the group as depicted in Table II, application no. 18, column 5 and 8, or a homolog or a fragment thereof.

The process of the invention comprises in one embodiment the following steps:
(i) increasing or generating of the expression of; and/or
(ii) increasing or generating the expression of an expression product of; and/or
(iii) increasing or generating one or more activities of an expression product encoded by; at least one nucleic acid molecule (in the following “Fine Chemical Related Protein (FCRP)”-encoding gene or “FCRP”-gene) comprising a nucleic acid molecule selected from the group consisting of:
(a) a nucleic acid molecule encoding the polypeptide shown in column 5 or 8 of Table II, preferably Table II B, application no. 18, or a homolog or a fragment thereof;
(b) a nucleic acid molecule shown in column 5 or 8 of Table I, preferably Table I B, application no. 18, or a homolog or a fragment thereof (preferably the coding region thereof);
(c) a nucleic acid molecule, which, as a result of the degeneracy of the genetic code, can be derived from a polypeptide sequence depicted in column 5 or 8 of Table II, preferably Table II B, application no. 18;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 18, or the coding region thereof;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d),
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 18;
(i) a nucleic acid molecule encoding a polypeptide having the activity represented by a respective protein as depicted in column 5 of Table II, application no. 18;
(j) a nucleic acid molecule which comprises a polynucleotide, which is obtained by amplifying a cDNA library or a genomic library using the primers as depicted in column 8 of Table III, application no. 18; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment thereof said nucleic acid molecule encodes a polypeptide which has the activity of the polypeptide represented by a protein comprising a polypeptide as depicted in the corresponding hit in column 5 of Table II, application no. 18.

In another preferred embodiment thereof said nucleic acid molecule confers a generation or an increase of the respective fine chemical in a non-human organism or a part thereof as compared to a corresponding wild-type non-human organism or part thereof.

In another preferred embodiment thereof said nucleic acid molecule encodes a polypeptide which has the activity of the polypeptide represented by a protein as depicted in the corresponding hit in column 5 of Table II, application no. 18, and confers a generation or an increase of the respective fine chemical in a non-human organism or a part thereof as compared to a corresponding wild-type non-human organism or part thereof.

Accordingly, the genes of the present invention or used in accordance with the present invention, which respectively encode a protein having an activity of 2,3-dihydroxy-2,3-dihydrophenylpropionatedehydrogenase, ABC metal ion transporter substrate-binding protein, acetyl-coenzyme A synthetase, acetyltransferase, acid shock protein, acyl transferase, agmatinase, aminotransferase, ATP-binding component of a transport system, b1522-protein, b3083-protein, b3644-protein, b3812-protein, b4029-protein, binding-protein-dependent transport systems inner membrane component, calcium-dependent protein kinase, cardiolipin synthase, chaperone protein CIpB, cyclin D, cystathionine gamma-synthase, eukaryotic translation initiation factor 5, geranylgeranyl pyrophosphate synthase, gibberellin 20-oxidase, glutamate-ammonia-ligase, glutaredoxin, glycosyl transferase, heat shock transcription factor, hydrolase, inositol-3-phosphate synthase, isochorismate synthase, leucyl/phenylalanyl-tRNA-protein transferase, major facilitator superfamily transporter protein, malate dehydrogenase, mevalonate kinase, mitochondrial inner membrane protease subunit, monodehydroascorbate reductase, multidrug resistance protein, NADH dehydrogenase subunit N, O-antigen chain length determinant, oxidoreductase, peroxisome assembly protein, potassium transport protein, protein kinase, purine nucleoside phosphorylase, rubredoxin, Sec-independent protein translocase subunit, serine protease, sll0064-protein, slr0383-protein, sodium/proton antiporter, thioredoxin family protein, threonine synthase, tryptophan synthase alpha chain, TTC0768-protein, TTC1386-protein, uroporphyrin-III C-methyltransferase, yer152c-protein, ylr065c-protein, Yml093w-protein, or zinc finger protein, which respectively encode a protein comprising a polypeptide encoded by a nucleic acid sequence as shown in Table I, application no. 18, column 5 or 8, (preferably the coding region thereof), or a homolog or a fragment thereof, which respectively encode a protein comprising a polypeptide as depicted in Table II, application no. 18, column 5 or 8, or a homolg or a fragment thereof, and/or which respectively can be amplified with the primer set shown in Table III, application no. 18, column 8, are also referred to as “FCRP genes”.

Proteins or polypeptides encoded by “FCRP-genes” are referred to as “Fine Chemical Related Proteins” or “FCRP”. For the purposes of the description of the present invention, the respective protein having an activity of 2,3-dihydroxy-2,3-dihydrophenylpropionatedehydrogenase, ABC metal ion transporter substrate-binding protein, acetyl-coenzyme A synthetase, acetyltransferase, acid shock protein, acyl transferase, agmatinase, aminotransferase, ATP-binding component of a transport system, b1522-protein, b3083-protein, b3644-protein, b3812-protein, b4029-protein, binding-protein-dependent transport systems inner membrane component, calcium-dependent protein kinase, cardiolipin synthase, chaperone protein CIpB, cyclin D, cystathionine gamma-synthase, eukaryotic translation initiation factor 5, geranylgeranyl pyrophosphate synthase, gibberellin 20-oxidase, glutamate-ammonia-ligase, glutaredoxin, glycosyl transferase, heat shock transcription factor, hydrolase, inositol-3-phosphate synthase, isochorismate synthase, leucyl/phenylalanyl-tRNA-protein transferase, major facilitator superfamily transporter protein, malate dehydrogenase, mevalonate kinase, mitochondrial inner membrane protease subunit, monodehydroascorbate reductase, multidrug resistance protein, NADH dehydrogenase subunit N, O-antigen chain length determinant, oxidoreductase, peroxisome assembly protein, potassium transport protein, protein kinase, purine nucleoside phosphorylase, rubredoxin, Sec-independent protein translocase subunit, serine protease, sll0064-protein, slr0383-protein, sodium/proton antiporter, thioredoxin family protein, threonine synthase, tryptophan synthase alpha chain, TTC0768-protein, TTC1386-protein, uroporphyrin-III C-methyltransferase, yer152c-protein, ylr065c-protein, yml093w-protein, or zinc finger protein, the respective protein comprising a polypeptide encoded by one or more respective nucleic acid sequences as shown in Table I, application no. 18, column 5 or 8, (preferably the coding region thereof), or a homolog or fragment thereof, the respective protein comprising a respective polypeptide as depicted in Table II, application no. 18, column 5 or 8, or a homolog or fragment thereof, the respective protein comprising a sequence corresponding to the consensus sequence as shown in Table IV, application no. 18, column 8, and/or the respective protein comprising at least one polypeptide motif as shown in Table IV, application no. 18, column 8 is also referred to as Fine Chemical Related Protein” or “FCRP”.

Thus, in one embodiment, the present invention provides a process of the production of cerotic acid, heneicosanoic acid, lignoceric acid, melissic acid and/or montanic acid, by increasing or generating one or more activities, especially selected from the group consisting of 2,3-dihydroxy-2,3-dihydrophenylpropionatedehydrogenase, ABC metal ion transporter substrate-binding protein, acetyl-coenzyme A synthetase, acetyltransferase, acid shock protein, acyl transferase, agmatinase, aminotransferase, ATP-binding component of a transport system, b1522-protein, b3083-protein, b3644-protein, b3812-protein, b4029-protein, binding-protein dependent transport systems inner membrane component, calcium-dependent protein kinase, cardiolipin synthase, chaperone protein CIpB, cyclin D, cystathionine gamma-synthase, eukaryotic translation initiation factor 5, geranylgeranyl pyrophosphate synthase, gibberellin 20-oxidase, glutamate-ammonia-ligase, glutaredoxin, glycosyl transferase, heat shock transcription factor, hydrolase, inositol-3-phosphate synthase, isochorismate synthase, leucyl/phenylalanyl-tRNA-protein transferase, major facilitator superfamily transporter protein, malate dehydrogenase, mevalonate kinase, mitochondrial inner membrane protease subunit, monodehydroascorbate reductase, multidrug resistance protein, NADH dehydrogenase subunit N, O-antigen chain length determinant, oxidoreductase, peroxisome assembly protein, potassium transport protein, protein kinase, purine nucleoside phosphorylase, rubredoxin, Sec-independent protein translocase subunit, serine protease, sll0064-protein, slr0383-protein, sodium/proton antiporter, thioredoxin family protein, threonine synthase, tryptophan synthase alpha chain, TTC0768-protein, TTC1386-protein, uroporphyrin-III C-methyltransferase, yer152c-protein, ylr065c-protein, yml093w-protein, and zinc finger protein, which is conferred by one or more FCRPs or the gene product of one or more FCRP-genes, for example by the gene product of a nucleic acid sequences comprising a polynucleotide selected from the group as shown in Table I, application no. 18, column 5 or 8, (preferably by the coding region thereof), or a homolog or a fragment thereof, e.g. or by one or more proteins each comprising a polypeptide encoded by one or more nucleic acid sequences selected from the group as shown in Table I, application no. 18, column 5 or 8, (preferably by the coding region thereof), or a homolog or a fragment thereof, or by one or more protein(s) each comprising a polypeptide selected from the group as depicted in Table II, application no. 18, column 5 and 8, or a homolog thereof, or a protein comprising a sequence corresponding to the consensus sequence or comprising at least one polypeptide motif as shown in Table IV, application no. 18, column 8.

for the disclosure of this paragraph see [0025.1.7.7] above

In an embodiment, the process comprises increasing or generating the activity of one or more polypeptides having said activity, e.g. by generating or increasing the amount and/or specific activity in the cell or a compartment of a cell of one of more FCRP, especially selected from the group consisting of 2,3-dihydroxy-2,3-dihydrophenylpropionatedehydrogenase, ABC metal ion transporter substrate-binding protein, acetyl-coenzyme A synthetase, acetyltransferase, acid shock protein, acyl transferase, agmatinase, aminotransferase, ATP-binding component of a transport system, b1522-protein, b3083-protein, b3644-protein, b3812-protein, b4029-protein, binding-protein-dependent transport systems inner membrane component, calcium-dependent protein kinase, cardiolipin synthase, chaperone protein CIpB, cyclin D, cystathionine gamma-synthase, eukaryotic translation initiation factor 5, geranylgeranyl pyrophosphate synthase, gibberellin 20-oxidase, glutamate-ammonia-ligase, glutaredoxin, glycosyl transferase, heat shock transcription factor, hydrolase, inositol-3-phosphate synthase, isochorismate synthase, leucyl/phenylalanyl-tRNA-protein transferase, major facilitator superfamily transporter protein, malate dehydrogenase, mevalonate kinase, mitochondrial inner membrane protease subunit, monodehydroascorbate reductase, multidrug resistance protein, NADH dehydrogenase subunit N, O-antigen chain length determinant, oxidoreductase, peroxisome assembly protein, potassium transport protein, protein kinase, purine nucleoside phosphorylase, rubredoxin, Sec-independent protein translocase subunit, serine protease, sll0064-protein, slr0383-protein, sodium/proton antiporter, thioredoxin family protein, threonine synthase, tryptophan synthase alpha chain, TTC0768-protein, TTC1386-protein, uroporphyrin-III C-methyltransferase, yer152c-protein, ylr065c-protein, yml093w-protein, and zinc finger protein, for example of the respective polypeptide as depicted in Table II, application no. 18, column 5 and 8, or a homolog or a fragment thereof, or the respective polypeptide comprising a sequence corresponding to the consensus sequences as shown in Table IV, application no. 18, column 8, or the respective polypeptide comprising at least one polypeptide motif as depicted in Table IV, application no. 18, column 8.

A further embodiment of the present invention relates to a process for the production of lignoceric acid, which comprises
(a) increasing or generating the activity of a eukaryotic translation initiation factor 5 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a thioredoxin family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a monodehydroascorbate reductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a glycosyl transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a gibberellin 20-oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a zinc finger protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a aminotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a glutamate-ammonia-ligase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a chaperone protein CIpB non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a purine nucleoside phosphorylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a threonine synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a serine protease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a ATP-binding component of a transport system non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a isochorismate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a leucyl/phenylalanyl-tRNA-protein transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a major facilitator superfamily transporter protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of cerotic acid, which comprises
(a) increasing or generating the activity of a multidrug resistance protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerotic acid or a composition comprising cerotic acid in said non-human organism or in the culture medium surrounding said non-human organism.

A further embodiment of the present invention relates to a process for the production of lignoceric acid, which comprises
(a) increasing or generating the activity of a sodium/proton antiporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a cardiolipin synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a b1522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of cerotic acid, which comprises
(a) increasing or generating the activity of a acid shock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerotic acid or a composition comprising cerotic acid in said non-human organism or in the culture medium surrounding said non-human organism.

A further embodiment of the present invention relates to a process for the production of lignoceric acid, which comprises
(a) increasing or generating the activity of a acid shock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a O-antigen chain length determinant non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a NADH dehydrogenase subunit N non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a 2,3-dihydroxy-2,3-dihydrophenylpropionatedehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a b3083-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a b3812-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a b4029-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a sll0064-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a agmatinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a uroporphyrin-III C-methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a binding-protein-dependent transport systems inner membrane component non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a malate dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a ABC metal ion transporter substrate-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a acyl transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a slr0383-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a geranylgeranyl pyrophosphate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a tryptophan synthase alpha chain non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a rubredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a Sec-independent protein translocase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a TTC0768-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a TTC1386-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a acetyl-coenzyme A synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a peroxisome assembly protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of heneicosanoic acid, which comprises
(a) increasing or generating the activity of a inositol-3-phosphate synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of heneicosanoic acid or a composition comprising heneicosanoic acid in said non-human organism or in the culture medium surrounding said non-human organism.

A further embodiment of the present invention relates to a process for the production of lignoceric acid, which comprises
(a) increasing or generating the activity of a cystathionine gamma-synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a potassium transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a ylr065c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a yml093w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a mitochondrial inner membrane protease subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating the activity of a mevalonate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of melissic acid which comprises
(a) increasing or generating the activity of a yer152c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of melissic acid or a composition comprising melissic acid in said non-human organism or in the culture medium surrounding said non-human organism.

A further embodiment of the present invention relates to a process for the production of montanic acid, which comprises
(a) increasing or generating the activity of a yer152c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of montanic acid or a composition comprising montanic acid in said non-human organism or in the culture medium surrounding said non-human organism.

A further embodiment of the present invention relates to a process for the production of melissic acid which comprises
(a) increasing or generating the activity of a b3644-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of melissic acid or a composition comprising melissic acid in said non-human organism or in the culture medium surrounding said non-human organism.

A further embodiment of the present invention relates to a process for the production of montanic acid, which comprises
(a) increasing or generating the activity of a b3644-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of montanic acid or a composition comprising montanic acid in said non-human organism or in the culture medium surrounding said non-human organism.

A further embodiment of the present invention relates to a process for the production of cerotic acid, which comprises
(a) increasing or generating the activity of a polypeptide comprising a polypeptide as depicted in the respective line in column 5 or 8 of Table II, application no. 18, or a homolog or a fragment thereof, a consensus sequence or at least one polypeptide motif as depicted in the respective line in column 8 of Table IV, application no. 18, whereby the respective line disclose in column 7 the fine chemical cerotic acid; or
increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, whereby the respective line disclose in column 7 the fine chemical cerotic acid;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of cerotic acid, or a composition comprising cerotic acid in said non-human organism or in the culture medium surrounding said non-human organism.

A further embodiment of the present invention relates to a process for the production of heneicosanoic acid, which comprises
(a) increasing or generating the activity of a polypeptide comprising a polypeptide as depicted in the respective line in column 5 or 8 of Table II, application no. 18, or a homolog or a fragment thereof, a consensus sequence or at least one polypeptide motif as depicted in the respective line in column 8 of Table IV, application no. 18, whereby the respective line disclose in column 7 the fine chemical heneicosanoic acid; or
increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, whereby the respective line disclose in column 7 the fine chemical heneicosanoic acid;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of heneicosanoic acid, or a composition comprising heneicosanoic acid in said non-human organism or in the culture medium surrounding said non-human organism.

A further embodiment of the present invention relates to a process for the production of lignoceric acid, which comprises
(a) increasing or generating the activity of a polypeptide comprising a polypeptide as depicted in the respective line in column 5 or 8 of Table II, application no. 18, or a homolog or a fragment thereof, a consensus sequence or at least one polypeptide motif as depicted in the respective line in column 8 of Table IV, application no. 18, whereby the respective line disclose in column 7 the fine chemical lignoceric acid; or
increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, whereby the respective line disclose in column 7 the fine chemical lignoceric acid;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of lignoceric acid, or a composition comprising lignoceric acid in said non-human organism or in the culture medium surrounding said non-human organism.

A further embodiment of the present invention relates to a process for the production of melissic acid which comprises
(a) increasing or generating the activity of a polypeptide comprising a polypeptide as depicted in the respective line in column 5 or 8 of Table II, application no. 18, or a homolog or a fragment thereof, a consensus sequence or at least one polypeptide motif as depicted in the respective line in column 8 of Table IV, application no. 18, whereby the respective line disclose in column 7 the fine chemical melissic acid; or
increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, whereby the respective line disclose in column 7 the fine chemical melissic acid;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of melissic acid or a composition comprising melissic acid in said non-human organism or in the culture medium surrounding said non-human organism.

A further embodiment of the present invention relates to a process for the production of montanic acid, which comprises
(a) increasing or generating the activity of a polypeptide comprising a polypeptide as depicted in the respective line in column 5 or 8 of Table II, application no. 18, or a homolog or a fragment thereof, a consensus sequence or at least one polypeptide motif as depicted in the respective line in column 8 of Table IV, application no. 18, whereby the respective line disclose in column 7 the fine chemical montanic acid; or
increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, whereby the respective line disclose in column 7 the fine chemical montanic acid;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of montanic acid, or a composition comprising montanic acid in said non-human organism or in the culture medium surrounding said non-human organism.

A further embodiment of the present invention relates to a process for the production of lignoceric acid, which comprises
(a) increasing or generating one or more activities selected from the group consisting of eukaryotic translation initiation factor 5 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of thioredoxin family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of monodehydroascorbate reductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of glycosyl transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of gibberellin 20-oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of zinc finger protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of aminotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of glutamate-ammonia-ligase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of chaperone protein CIpB in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of purine nucleoside phosphorylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of threonine synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of serine protease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of ATP-binding component of a transport system in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of isochorismate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of leucyl/phenylalanyl-tRNA-protein transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of major facilitator superfamily transporter protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of cerotic acid, which comprises
(a) increasing or generating one or more activities selected from the group consisting of multidrug resistance protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerotic acid or a composition comprising cerotic acid in said non-human organism or in the culture medium surrounding said non-human organism.

A further embodiment of the present invention relates to a process for the production of lignoceric acid, which comprises
(a) increasing or generating one or more activities selected from the group consisting of sodium/proton antiporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of cardiolipin synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of b1522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of cerotic acid, which comprises
(a) increasing or generating one or more activities selected from the group consisting of acid shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerotic acid or a composition comprising cerotic acid in said non-human organism or in the culture medium surrounding said non-human organism.

A further embodiment of the present invention relates to a process for the production of lignoceric acid, which comprises
(a) increasing or generating one or more activities selected from the group consisting of acid shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of O-antigen chain length determinant in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of NADH dehydrogenase subunit N in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of 2,3-dihydroxy-2,3-dihydrophenylpropionatedehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of b3083-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of b3812-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of b4029-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of sll0064-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of agmatinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of uroporphyrin-III C-methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of binding-protein-dependent transport systems inner membrane component in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of malate dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of ABC metal ion transporter substrate-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of acyl transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of slr0383-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of geranylgeranyl pyrophosphate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of tryptophan synthase alpha chain in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of rubredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of Sec-independent protein translocase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of TTC0768-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of TTC1386-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of acetyl-coenzyme A synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of peroxisome assembly protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of heneicosanoic acid, which comprises
(a) increasing or generating one or more activities selected from the group consisting of inositol-3-phosphate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of heneicosanoic acid or a composition comprising heneicosanoic acid in said non-human organism or in the culture medium surrounding said non-human organism.

A further embodiment of the present invention relates to a process for the production of lignoceric acid, which comprises
(a) increasing or generating one or more activities selected from the group consisting of cystathionine gamma-synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of potassium transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of ylr065c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of yml093w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of mitochondrial inner membrane protease subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of lignoceric acid, which comprises
(a) increasing or generating one or more activities selected from the group consisting of mevalonate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

A further embodiment of the present invention relates to a process for the production of melissic acid which comprises
(a) increasing or generating one or more activities selected from the group consisting of yer152c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of melissic acid or a composition comprising melissic acid in said non-human organism or in the culture medium surrounding said non-human organism.

A further embodiment of the present invention relates to a process for the production of montanic acid, which comprises
(a) increasing or generating one or more activities selected from the group consisting of yer152c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of montanic acid or a composition comprising montanic acid in said non-human organism or in the culture medium surrounding said non-human organism.

A further embodiment of the present invention relates to a process for the production of melissic acid which comprises
(a) increasing or generating one or more activities selected from the group consisting of b3644-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of melissic acid or a composition comprising melissic acid in said non-human organism or in the culture medium surrounding said non-human organism.

A further embodiment of the present invention relates to a process for the production of montanic acid, which comprises
(a) increasing or generating one or more activities selected from the group consisting of b3644-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of montanic acid or a composition comprising montanic acid in said non-human organism or in the culture medium surrounding said non-human organism.

A further embodiment of the present invention relates to a process for the production of cerotic acid, which comprises
(a1) increasing or generating the activity of a polypeptide comprising a polypeptide as depicted in the respective line in column 5 or 8 of Table II, application no. 18, or a homolog or fragment thereof, a consensus sequence or at least one polypeptide motif as depicted in column 8 of Table IV, application no. 18, whereby the respective line disclose in column 7 the fine chemical cerotic acid; or
increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, whereby the respective line disclose in column 7 the fine chemical cerotic acid;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 18, or a homolog or a fragment thereof, a consensus sequence or at least one polypeptide motif as depicted in the respective line in column 8 of Table IV, application no. 18, which is joined to a transit peptide, whereby the respective line disclose in column 7 the fine chemical cerotic acid; or
increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical cerotic acid;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 18, or a homolog or a fragment thereof, a consensus sequence or at least one polypeptide motif as depicted in the respective line in column 8 of Table IV, application no. 18, whereby the respective line disclose in column 7 the fine chemical cerotic acid; or
increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, whereby the respective line disclose in column 7 the fine chemical cerotic acid;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of cerotic acid, or a composition comprising cerotic acid in said non-human organism or in the culture medium surrounding said non-human organism.

A further embodiment of the present invention relates to a process for the production of heneicosanoic acid, which comprises
(a1) increasing or generating the activity of a polypeptide comprising a polypeptide as depicted in the respective line in column 5 or 8 of Table II, application no. 18, or a homolog or fragment thereof, a consensus sequence or at least one polypeptide motif as depicted in column 8 of Table IV, application no. 18, whereby the respective line disclose in column 7 the fine chemical heneicosanoic acid; or
increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, whereby the respective line disclose in column 7 the fine chemical heneicosanoic acid;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 18, or a homolog or a fragment thereof, a consensus sequence or at least one polypeptide motif as depicted in the respective line in column 8 of Table IV, application no. 18, which is joined to a transit peptide, whereby the respective line disclose in column 7 the fine chemical heneicosanoic acid; or
increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical heneicosanoic acid;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 18, or a homolog or a fragment thereof, a consensus sequence or at least one polypeptide motif as depicted in the respective line in column 8 of Table IV, application no. 18, whereby the respective line disclose in column 7 the fine chemical heneicosanoic acid; or
increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, whereby the respective line disclose in column 7 the fine chemical heneicosanoic acid;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of heneicosanoic acid, or a composition comprising heneicosanoic acid in said non-human organism or in the culture medium surrounding said non-human organism.

A further embodiment of the present invention relates to a process for the production of lignoceric acid, which comprises
(a1) increasing or generating the activity of a polypeptide comprising a polypeptide as depicted in the respective line in column 5 or 8 of Table II, application no. 18, or a homolog or fragment thereof, a consensus sequence or at least one polypeptide motif as depicted in column 8 of Table IV, application no. 18, whereby the respective line disclose in column 7 the fine chemical lignoceric acid; or
increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, whereby the respective line disclose in column 7 the fine chemical lignoceric acid;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 18, or a homolog or a fragment thereof, a consensus sequence or at least one polypeptide motif as depicted in the respective line in column 8 of Table IV, application no. 18, which is joined to a transit peptide, whereby the respective line disclose in column 7 the fine chemical lignoceric acid; or
increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical lignoceric acid;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 18, or a homolog or a fragment thereof, a consensus sequence or at least one polypeptide motif as depicted in the respective line in column 8 of Table IV, application no. 18, whereby the respective line disclose in column 7 the fine chemical lignoceric acid; or
increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, whereby the respective line disclose in column 7 the fine chemical lignoceric acid;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of lignoceric acid, or a composition comprising lignoceric acid in said non-human organism or in the culture medium surrounding said non-human organism.

A further embodiment of the present invention relates to a process for the production of melissic acid which comprises
(a1) increasing or generating the activity of a polypeptide comprising a polypeptide as depicted in the respective line in column 5 or 8 of Table II, application no. 18, or a homolog or fragment thereof, a consensus sequence or at least one polypeptide motif as depicted in column 8 of Table IV, application no. 18, whereby the respective line disclose in column 7 the fine chemical melissic acid; or
increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, whereby the respective line disclose in column 7 the fine chemical melissic acid;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 18, or a homolog or a fragment thereof, a consensus sequence or at least one polypeptide motif as depicted in the respective line in column 8 of Table IV, application no. 18, which is joined to a transit peptide, whereby the respective line disclose in column 7 the fine chemical melissic acid; or
increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical melissic acid;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 18, or a homolog or a fragment thereof, a consensus sequence or at least one polypeptide motif as depicted in the respective line in column 8 of Table IV, application no. 18, whereby the respective line disclose in column 7 the fine chemical melissic acid; or
increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, whereby the respective line disclose in column 7 the fine chemical melissic acid;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of melissic acid or a composition comprising melissic acid in said non-human organism or in the culture medium surrounding said non-human organism.

A further embodiment of the present invention relates to a process for the production of montanic acid, which comprises
(a1) increasing or generating the activity of a polypeptide comprising a polypeptide as depicted in the respective line in column 5 or 8 of Table II, application no. 18, or a homolog or fragment thereof, a consensus sequence or at least one polypeptide motif as depicted in column 8 of Table IV, application no. 18, whereby the respective line disclose in column 7 the fine chemical montanic acid; or
increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, whereby the respective line disclose in column 7 the fine chemical montanic acid;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 18, or a homolog or a fragment thereof, a consensus sequence or at least one polypeptide motif as depicted in the respective line in column 8 of Table IV, application no. 18, which is joined to a transit peptide, whereby the respective line disclose in column 7 the fine chemical montanic acid; or
increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical montanic acid;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 18, or a homolog or a fragment thereof, a consensus sequence or at least one polypeptide motif as depicted in the respective line in column 8 of Table IV, application no. 18, whereby the respective line disclose in column 7 the fine chemical montanic acid; or
increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, whereby the respective line disclose in column 7 the fine chemical montanic acid;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
and
(b) growing the non-human organism under conditions which permit the production of montanic acid, or a composition comprising montanic acid in said non-human organism or in the culture medium surrounding said non-human organism.

Accordingly, the present invention relates to a process for the production of lignoceric acid, which comprises
(a) increasing or generating the activity of a eukaryotic translation initiation factor 5 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a thioredoxin family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a monodehydroascorbate reductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a glycosyl transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a gibberellin 20-oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a zinc finger protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a aminotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a glutamate-ammonia-ligase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a chaperone protein CIpB in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a purine nucleoside phosphorylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a threonine synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a serine protease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a ATP-binding component of a transport system in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a isochorismate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a leucyl/phenylalanyl-tRNA-protein transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a major facilitator superfamily transporter protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of cerotic acid, which comprises
(a) increasing or generating the activity of a multidrug resistance protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerotic acid or a composition comprising cerotic acid in said non-human organism or in the culture medium surrounding said non-human organism.

Accordingly, the present invention relates to a process for the production of lignoceric acid, which comprises
(a) increasing or generating the activity of a sodium/proton antiporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a cardiolipin synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a b1522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of cerotic acid, which comprises
(a) increasing or generating the activity of a acid shock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerotic acid or a composition comprising cerotic acid in said non-human organism or in the culture medium surrounding said non-human organism.

Accordingly, the present invention relates to a process for the production of lignoceric acid, which comprises
(a) increasing or generating the activity of a acid shock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a O-antigen chain length determinant in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a NADH dehydrogenase subunit N in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a 2,3-dihydroxy-2,3-dihydrophenylpropionatedehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a b3083-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a b3812-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a b4029-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a sll0064-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a agmatinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a uroporphyrin-III C-methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a binding-protein-dependent transport systems inner membrane component in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a malate dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a ABC metal ion transporter substrate-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a acyl transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a slr0383-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a geranylgeranyl pyrophosphate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a tryptophan synthase alpha chain in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a rubredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a Sec-independent protein translocase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a TTC0768-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a TTC1386-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a acetyl-coenzyme A synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a peroxisome assembly protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of heneicosanoic acid, which comprises
(a) increasing or generating the activity of a inositol-3-phosphate synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of heneicosanoic acid or a composition comprising heneicosanoic acid in said non-human organism or in the culture medium surrounding said non-human organism.

Accordingly, the present invention relates to a process for the production of lignoceric acid, which comprises
(a) increasing or generating the activity of a cystathionine gamma-synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a potassium transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a ylr065c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a yml093w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a mitochondrial inner membrane protease subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of lignoceric acid, which comprises
(a) increasing or generating the activity of a mevalonate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of lignoceric acid or a composition comprising lignoceric acid in said non-human organism or in the culture medium surrounding said non-human organism.

Accordingly, the present invention relates to a process for the production of melissic acid which comprises
(a) increasing or generating the activity of a yer152c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of melissic acid or a composition comprising melissic acid in said non-human organism or in the culture medium surrounding said non-human organism.

Accordingly, the present invention relates to a process for the production of montanic acid, which comprises
(a) increasing or generating the activity of a yer152c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of montanic acid or a composition comprising montanic acid in said non-human organism or in the culture medium surrounding said non-human organism.

Accordingly, the present invention relates to a process for the production of melissic acid which comprises
(a) increasing or generating the activity of a b3644-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of melissic acid or a composition comprising melissic acid in said non-human organism or in the culture medium surrounding said non-human organism.

Accordingly, the present invention relates to a process for the production of montanic acid, which comprises
(a) increasing or generating the activity of a b3644-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of montanic acid or a composition comprising montanic acid in said non-human organism or in the culture medium surrounding said non-human organism.

Accordingly, the present invention relates to a process for the production of cerotic acid, which comprises
(a) increasing or generating the activity of a polypeptide comprising a polypeptide as depicted in the respective line in column 5 or 8 of Table II, application no. 18, or a homolog or a fragment thereof, a consensus sequence or at least one polypeptide motif as depicted in the respective line in column 8 of Table IV, application no. 18, whereby the respective line disclose in column 7 the fine chemical cerotic acid; or
increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, whereby the respective line disclose in column 7 the fine chemical cerotic acid;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of cerotic acid, or a composition comprising cerotic acid in said non-human organism or in the culture medium surrounding said non-human organism.

Accordingly, the present invention relates to a process for the production of heneicosanoic acid, which comprises
(a) increasing or generating the activity of a polypeptide comprising a polypeptide as depicted in the respective line in column 5 or 8 of Table II, application no. 18, or a homolog or a fragment thereof, a consensus sequence or at least one polypeptide motif as depicted in the respective line in column 8 of Table IV, application no. 18, whereby the respective line disclose in column 7 the fine chemical heneicosanoic acid; or
increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, whereby the respective line disclose in column 7 the fine chemical heneicosanoic acid;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of heneicosanoic acid, or a composition comprising heneicosanoic acid in said non-human organism or in the culture medium surrounding said non-human organism.

Accordingly, the present invention relates to a process for the production of lignoceric acid, which comprises
(a) increasing or generating the activity of a polypeptide comprising a polypeptide as depicted in the respective line in column 5 or 8 of Table II, application no. 18, or a homolog or a fragment thereof, a consensus sequence or at least one polypeptide motif as depicted in the respective line in column 8 of Table IV, application no. 18, whereby the respective line disclose in column 7 the fine chemical lignoceric acid; or
increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, whereby the respective line disclose in column 7 the fine chemical lignoceric acid;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of lignoceric acid, or a composition comprising lignoceric acid in said non-human organism or in the culture medium surrounding said non-human organism.

Accordingly, the present invention relates to a process for the production of melissic acid which comprises
(a) increasing or generating the activity of a polypeptide comprising a polypeptide as depicted in the respective line in column 5 or 8 of Table II, application no. 18, or a homolog or a fragment thereof, a consensus sequence or at least one polypeptide motif as depicted in the respective line in column 8 of Table IV, application no. 18, whereby the respective line disclose in column 7 the fine chemical melissic acid; or
increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, whereby the respective line disclose in column 7 the fine chemical melissic acid;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of melissic acid or a composition comprising melissic acid in said non-human organism or in the culture medium surrounding said non-human organism.

Accordingly, the present invention relates to a process for the production of montanic acid, which comprises
(a) increasing or generating the activity of a polypeptide comprising a polypeptide as depicted in the respective line in column 5 or 8 of Table II, application no. 18, or a homolog or a fragment thereof, a consensus sequence or at least one polypeptide motif as depicted in the respective line in column 8 of Table IV, application no. 18, whereby the respective line disclose in column 7 the fine chemical montanic acid; or
increasing or generating the activity of an expression product of one or more nucleic acid molecule(s) comprising a polynucleotide as depicted in the respective line in column 5 or 8 of Table I, application no. 18, preferably the coding region thereof, or a homolog or a fragment thereof, whereby the respective line disclose in column 7 the fine chemical montanic acid;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of montanic acid, or a composition comprising montanic acid in said non-human organism or in the culture medium surrounding said non-human organism.

In a further embodiment the activity of the polypeptide comprising a polypeptide as depicted in the respective line in column 5 or 8 of Table II, application no. 18, or a homolog or a fragment thereof, a consensus sequence or at least one polypeptide motif as depicted in the respective line in column 8 of Table IV, application no. 18, is increased or generated non-targeted in the above-mentioned process in a microorganism or plant or a part thereof.

In a further embodiment said polypeptide has the activity of the respective polypeptide represented by a protein comprising a polypeptide as depicted in the respective line in column 5 of Table II, application no. 18.

In a further embodiment the activity of the expression product of one or more nucleic acid molecule(s) comprising a polynucleotide as depicted in the respective line in column 5 or 8 of Table I, application no. 18, preferably the coding region thereof, or a homolog or a fragment thereof, is increased or generated non-targeted in the above-mentioned process in a microorganism or plant or a part thereof.

In a further embodiment the activity of the polypeptide comprising a polypeptide as depicted in the respective line in column 5 or 8 of Table II, application no. 18, or a homolog or a fragment thereof, a consensus sequence or at least one polypeptide motif as depicted in the respective line in column 8 of Table IV, application no. 18, is increased or generated in the above-mentioned process in an organelle preferably in plastids or mitochondria, especially in plastids, of a microorganism or plant.

In a further embodiment said polypeptide has the activity of the respective polypeptide represented by a protein comprising a polypeptide as depicted in the respective line in column 5 of Table II, application no. 18.

In a further embodiment the activity of the expression product of one or more nucleic acid molecule(s) comprising a polynucleotide as depicted in the respective line in column 5 or 8 of Table I, application no. 18, preferably the coding region thereof, or a homolog or a fragment thereof, is increased or generated in the above-mentioned process in an organelle, preferably in plasids or mitochondria, especially in plastids, of a microorganism or plant.

In a further embodiment the activity of the polypeptide comprising a polypeptide as depicted in the respective line in column 5 or 8 of Table II, application no. 18, or a homolog or a fragment thereof, a consensus sequence or at least one polypeptide motif as depicted in the respective line in column 8 of Table IV, application no. 18, is increased or generated in the above-mentioned process in the cyctosol of a cell, of a microorganism or plant.

In a further embodiment said polypeptide has the activity of the respective polypeptide represented by a protein comprising a polypeptide as depicted in the respective line in column 5 of Table II, application no. 18.

In a further embodiment the activity of the expression product of one or more nucleic acid molecule(s) comprising a polynucleotide as depicted in the respective line in column 5 or 8 of Table I, application no. 18, preferably the coding region thereof, or a homolog or a fragment thereof, is increased or generated in the above-mentioned process in the cytosol of a cell, of a microorganism or plant.

[0039.1.7.18] to [0066.1.7.18] for the disclosure of these paragraphs see [0039.1.7.7] to [0066.1.7.7] above

As mentioned above the nucleic acid sequences coding for the proteins as shown in Table II, application no. 18, column 5 or 8, or homologs or fragments thereof, are joined to a nucleic acid sequence encoding a plastidic transit peptide, e.g, if for the nucleic acid molecule in Table I, column 6 the term “plastidic” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide coding sequence is fused in frame to the nucleic acid sequence coding for proteins as shown in Table II, application no. 18, column 5 or 8, or homologs or fragments thereof, e.g., if for the nucleic acid molecule in Table I, column 6 the term “plastidic” is indicated.

[0068.1.7.18] to [0072.1.7.18] for the disclosure of these paragraphs see [0068.1.7.7] to [0072.1.7.7] above

As mentioned above the nucleic acid sequences coding for the proteins as shown in the respective line in Table II, application no. 18, column 5 or 8, or homologs or fragments thereof, are joined to a nucleic acid sequence encoding a mitochondric transit peptide, e.g., if for the respective nucleic acid molecule in Table I, column 6 the term “mitochondric” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in the respective line in Table II, application no. 18, column 5 or 8, or homologs or fragments thereof, e.g., if for the nucleic acid molecule in the respective line in Table I, column 6 the term “mitochondric” is indicated.

for the disclosure of this paragraph see [0074.1.7.7] above

for the disclosure of this paragraph see [0075.1.7.7] above

Nucleic acid sequences coding for the transit peptides may be chemically synthesized either in part or wholly according to structure of transit peptide sequences disclosed in the prior art. Said natural or chemically synthesized sequences can be directly linked to the sequences encoding the mature protein or via a linker nucleic acid sequence, which may be typically less than 500 base pairs, preferably less than 450, 400, 350, 300, 250 or 200 base pairs, more preferably less than 150, 100, 90, 80, 70, 60, 50, 40 or 30 base pairs and most preferably less than 25, 20, 15, 12, 9, 6 or 3 base pairs in length and are in frame to the coding sequence. Furthermore favorable nucleic acid sequences encoding transit peptides may comprise sequences derived from more than one biological and/or chemical source and may include a nucleic acid sequence derived from the amino-terminal region of the mature protein, which in its native state is linked to the transit peptide. In a preferred embodiment of the invention said amino-terminal region of the mature protein is typically less than 150 amino acids, preferably less than 140, 130, 120, 110, 100 or 90 amino acids, more preferably less than 80, 70, 60, 50, 40, 35, 30, 25 or 20 amino acids and most preferably less than 19, 18, 17, 16, 15, 14, 13, 12, 11 or 10 amino acids in length. But even shorter or longer stretches are also possible. In addition target sequences, which facilitate the transport of proteins to other cell compartments such as the vacuole, endoplasmic reticulum, golgi complex, glyoxysomes, peroxisomes, mitochondria (in case of a plastidal transit peptide) or plastids, especially chloroplasts (in case of a mitochondric transit peptide) may be also part of the inventive nucleic acid sequence. The proteins translated from said inventive nucleic acid sequences are a kind of fusion proteins that means the nucleic acid sequences encoding the plastidal transit peptide for example the ones shown in Table a, preferably the last one of the Table a are joint to the respective nucleic acid sequences shown in Table I, application no. 18, columns 5 or 8, especially the coding region thereof, or homologs or fragments thereof, in case of nucleic acid sequences being expressed in plastids, and that means the nucleic acid sequences encoding the mitochondric transit peptide for example the ones shown in Table b, preferably the last one of the Table b are joint to the respective nucleic acid sequences shown in Table I, application no. 18, columns 5 or 8, especially the coding region thereof, or homologs or fragments thereof, in case of nucleic acid sequences being expressed in mitochondria. The person skilled in the art is able to join said sequences in a functional manner. Advantageously the transit peptide part is cleaved off from the protein part shown in Table II, application no. 18, columns 5 or 8, or a homolog or fragment thereof, during or after the transport into the organelles, preferably into the plastids or mitochondria, respectively.

All products of the cleavage of the preferred transit peptide shown in the last line of Table a have preferably the N-terminal amino acid sequences QIA CSS or QIA EFQLTT in front of the start methionine of the respective protein mentioned in Table II, application no. 18, column 5 or 8. or homologs or fragments thereof. Other short amino acid sequences of an range of 1 to 20 amino acids preferable 2 to 15 amino acids, more preferable 3 to 10 amino acids most preferably 4 to 8 amino acids are also possible in front of the start methionine of the protein mentioned in Table II, application no. 18, columns 5 or 8, or homologs or fragments thereof. In case of the amino acid sequence QIA CSS the three amino acids in front of the start methionine are stemming from the LIC (=ligatation independent cloning) cassette. Said short amino acid sequence is preferred in the case of the expression of E. coli genes. In case of the amino acid sequence QIA EFQLTT the six amino acids in front of the start methionine are stemming from the LIC cassette. Said short amino acid sequence is preferred in the case of the expression of S. cerevisiae genes. The skilled worker knowns that other short sequences are also useful in the expression of the respective nucleic acid molecule comprising a polynucleotide as depicted in Table I, application no. 18, column 5 or 8, preferably the coding region thereof, or a homolog or a fragment thereof. Furthermore the skilled worker is aware of the fact that there is not a need for such short sequences in the expression of the genes.

for the disclosure of this paragraph see [0077.1.7.7] above

for the disclosure of this paragraph see [0078.1.7.7] above

Alternatively to the targeting of the respective sequences shown in Table II, application no. 18, columns 5 or 8, or homologs or fragments thereof, preferably of sequences in general encoded in the nucleus with the aid of the targeting sequences mentioned for example in Table a alone, Table b alone, or in combination with other targeting sequences preferably into plastids or mitochondria, respectively, the nucleic acids of the invention can directly be introduced into the plastidal or mitochondrial genome. Therefore in a preferred embodiment the respective nucleic acid sequences shown in the respective line in Table I, application no. 18, columns 5 or 8, preferably the coding region thereof, or homologs or fragments thereof, are directly introduced and expressed in plastids e.g., if for the nucleic acid molecule in the respective line in Table I, column 6, the term “plastidic” is indicated, or are directly introduced and expressed in mitochondria e.g., if for the nucleic acid molecule in the respective line in Table I, column 6 the term “mitochondric” is indicated. The term “introduced” in the context of this specification shall mean the insertion of a nucleic acid sequence into the organism by means of a “transfection”, “transduction” or preferably by “transformation”.

A plastid, such as a chloroplast, or a mitochondrion, has been “transformed” by an exogenous (preferably foreign) nucleic acid sequence if a nucleic acid sequence has been introduced into the plastid or mitochondrion that means that this sequence has crossed the membrane or the membranes of the plastid or of the mitochondrion. The exogenous, preferably foreign, DNA may be integrated (covalently linked) into plastid or mitochondrial DNA making up the genome of the plastid or mitochondrion, or it may remain unintegrated (e.g., by including a chloroplast origin of replication). “Stably” integrated DNA sequences are those, which are inherited through plastid or mitochondrion replication, thereby transferring new plastids or mitochondria, with the features of the integrated DNA sequence to the progeny.

[0080.1.7.18] to [0083.1.7.18] for the disclosure of these paragraphs see [0080.1.7.7] to [0083.1.7.7] above

For the process of the present invention it may be of great advantage that by transforming the plastids the intraspecies specific transgene flow is blocked, because a lot of species such as corn, cotton and rice have a strict maternal inheritance of plastids. By placing the respective genes specified in the respective line in Table I, application no. 18, columns 5 or 8, preferably the coding region, or homologs or fragments thereof, e.g. if for the nucleic acid molecule in the respective line in column 6 of Table I the term “plastidic” is indicated, in the plastids of plants, these genes will not be present in the pollen of said plants.

A further preferred embodiment of the invention relates to the use of so called “chloroplast localization sequences”, in which a first RNA sequence or molecule is capable of transporting or “chaperoning” a second RNA sequence, such as a RNA sequence transcribed from the sequences depicted in the respective line in Table I, application no. 18, columns 5 or 8, preferably the coding region thereof, or a homolog or a fragment thereof, or a sequence encoding a protein, as depicted in the respective line in Table II, application no. 18, columns 5 and 8, or a homolog or a fragment thereof, from an external environment inside a cell or outside a plastid into a chloroplast. In one embodiment the chloroplast localization signal is substantially similar or complementary to a complete or intact viroid sequence. The chloroplast localization signal may be encoded by a DNA sequence, which is transcribed into the chloroplast localization RNA. The term “viroid” refers to a naturally occurring single stranded RNA molecule (Flores, C. R. Acad. Sci. III. 324 (10), 943 (2001)). Viroids usually contain about 200-500 nucleotides and generally exist as circular molecules. Examples of viroids that contain chloroplast localization signals include but are not limited to ASBVd, PLMVd, CChMVd and ELVd. The viroid sequence or a functional part of it can be fused to the sequences depicted in the respective line in Table I, application no. 18, columns 5 or 8, preferably the coding region thereof, or a homolog or a fragment thereof, or a sequence encoding a protein, as depicted in the respective line in Table II, application no. 18, columns 5 or 8, or a homolog or a fragment thereof, in such a manner that the viroid sequence transports a sequence transcribed from a sequence as depicted in the respective line in Table I, application no. 18 columns 5 or 8, preferably the coding region thereof, or a homolog or a fragment thereof, or a sequence encoding a protein as depicted in the respective line in Table II, application no. 18 columns 5 or 8, or a homolog or a fragment thereof, into the chloroplasts, e.g. if for the nucleic acid molecule in column 6 of Table I the term “plastidic” is indicated. A preferred embodiment uses a modified ASBVd (Navarro et al., Virology. 268 (1), 218 (2000)).

In a further specific embodiment the protein to be expressed in the plastids such as the proteins depicted in the respective line in Table II, application no. 18, columns 5 or 8, or a homolog or a fragment thereof, e.g. if for the nucleic acid molecule in the respective line in column 6 of Table I the term “plastidic” is indicated, are encoded by different nucleic acids. Such a method is disclosed in WO 2004/040973, which shall be incorporated by reference. WO 2004/040973 teaches a method, which relates to the translocation of an RNA corresponding to a gene or gene fragment into the chloroplast by means of a chloroplast localization sequence. The genes, which should be expressed in the plant or plant cells, are split into nucleic acid fragments, which are introduced into different compartments in the plant e.g. the nucleus, the plastids and/or mitochondria. Additionally plant cells are described in which the chloroplast contains a ribozyme fused at one end to an RNA encoding a fragment of a protein used in the inventive process such that the ribozyme can trans-splice the translocated fusion RNA to the RNA encoding the gene fragment to form and as the case may be reunite the nucleic acid fragments to an intact mRNA encoding a functional protein for example as disclosed in the respective line in Table II, application no. 18, columns 5 or 8, or a homolog or a fragment thereof.

In a preferred embodiment of the invention the nucleic acid sequences as shown in the respective line in Table I, application no. 18, columns 5 or 8, preferably the coding region thereof, or homologs or fragments thereof, e.g. if for the nucleic acid molecule in the respective line in column 6 of Table I the term “plastidic” is indicated, used in the inventive process are transformed into plastids, which are metabolically active. Those plastids should preferably maintain at a high copy number in the plant or plant tissue of interest, most preferably the chloroplasts found in green plant tissues, such as leaves or cotyledons or in seeds.

For a good expression in the plastids the nucleic acid sequences as shown in the respective line in Table I, application no. 18, columns 5 or 8, preferably the coding region thereof, or homologs or fragments thereof, e.g. if for the nucleic acid molecule in the respective line in column 6 of Table I the term “plastidic” is indicated, are introduced into an expression cassette using preferably a promoter and terminater, which are active in plastids preferably a chloroplast promoter. Examples of such promoters include the psbA promoter from the gene from spinach or pea, the rbcL promoter, and the atpB promoter from corn.

[0089.1.7.18] to [0092.1.7.18] for the disclosure of these paragraphs see [0089.1.7.7] to [0092.1.7.7] above

Advantageously the process for the production of the fine chemical cerotic acid, heneicosanoic acid, lignoceric acid, melissic acid and/or montanic acid leads to an enhanced production of the respective fine chemical. The terms “enhanced” or “increase” mean at least a 10%, 20%, 30%, 40% or 50%, preferably at least 60%, 70%, 80%, 90% or 100%, more preferably 150%, 200%, 300%, 400% or 500% higher production of the respective fine chemical cerotic acid, heneicosanoic acid, lignoceric acid, melissic acid and/or montanic acid in comparison to the wild-type as defined above, e.g. that means in comparison to a non-human organism without the aforementioned modification of the activity of a protein as shown in the respective line in Table II, application no. 18, column 5 or 8, or a fragment or a homolog thereof. The modification of the activity of a protein as shown in the respective line in Table II, application no. 18, column 5 or 8, or a homolog or a fragment thereof, or their combination can be achieved by joining the protein to a respective transit peptide, e.g. if for the respective encoding nucleic acid molecule in column 6 of Table I the term “plastidic” or “mitochondric” is indicated.

Surprisingly it was found, that the transgenic expression of a protein as shown in the respective line in Table II, application no. 18, column 5, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, conferred a production of or an increase in cerotic acid, heneicosanoic acid, lignoceric acid, melissic acid and/or montanic acid,respectively, to the transgenic non-human organism as compared to a corresponding non-transformed wild type.

In one embodiment the transgenic expression of a protein as shown in the respective line in Table II, application no. 18, column 5 or 8, or a homolog or a fragment thereof, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, confers a production of or an increase in cerotic acid, heneicosanoic acid, lignoceric acid, melissic acid and/or montanic acid,respectively, to the transgenic non-human organism as compared to a corresponding non-transformed wild type.

Accordingly, in one embodiment, an increase of lignoceric acid in a non-human organism, as compared to a corresponding non-transformed wild type non-human organism, is conferred in the process of the invention, if the activity of a polypeptide showing the activity of a eukaryotic translation initiation factor 5, or if the activity of the polypeptide At1g36730, preferably represented by SEQ ID NO. 17969, or a homolog or fragment thereof, or if the activity of a polypeptide encoded by a nucleic acid molecule comprising the nucleic acid SEQ ID NO. 17968, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 17968 or polypeptide SEQ ID NO. 17969, respectively, or a homolog or a fragment thereof, is increased or generated, or if the activity eukaryotic translation initiation factor 5 is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 31 to 65-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a protein kinase, or if the activity of the polypeptide At1g48260, preferably represented by SEQ ID NO. 18236, or a homolog or fragment thereof, or if the activity of a polypeptide encoded by a nucleic acid molecule comprising the nucleic acid SEQ ID NO. 18235, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 18235 or polypeptide SEQ ID NO. 18236, respectively, or a homolog or a fragment thereof, is increased or generated, or if the activity protein kinase is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 33 to 86-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a thioredoxin family protein, or if the activity of the polypeptide At3g08710, preferably represented by SEQ ID NO. 22250, or a homolog or fragment thereof, or if the activity of a polypeptide encoded by a nucleic acid molecule comprising the nucleic acid SEQ ID NO. 22249, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 22249 or polypeptide SEQ ID NO. 22250, respectively, or a homolog or a fragment thereof, is increased or generated, or if the activity thioredoxin family protein is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 31 to 127-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a monodehydroascorbate reductase, or if the activity of the polypeptide At3g09940, preferably represented by SEQ ID NO. 152082, or a homolog or fragment thereof, or if the activity of a polypeptide encoded by a nucleic acid molecule comprising the nucleic acid SEQ ID NO. 152081, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 152081 or polypeptide SEQ ID NO. 152082, respectively, or a homolog or a fragment thereof, is increased or generated, or if the activity monodehydroascorbate reductase is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 28 to 36-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a glycosyl transferase, or if the activity of the polypeptide At3g27540, preferably represented by SEQ ID NO. 68778, or a homolog or fragment thereof, or if the activity of a polypeptide encoded by a nucleic acid molecule comprising the nucleic acid SEQ ID NO. 68777, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 68777 or polypeptide SEQ ID NO. 68778, respectively, or a homolog or a fragment thereof, is increased or generated, or if the activity glycosyl transferase is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 39 to 99-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a glutaredoxin, or if the activity of the polypeptide At3g62950, preferably represented by SEQ ID NO. 2936, or a homolog or fragment thereof, or if the activity of a polypeptide encoded by a nucleic acid molecule comprising the nucleic acid SEQ ID NO. 2935, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 2935 or polypeptide SEQ ID NO. 2936, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 30 to 159-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a heat shock transcription factor, or if the activity of the polypeptide At4g18880, preferably represented by SEQ ID NO. 24233, or a homolog or fragment thereof, or if the activity of a polypeptide encoded by a nucleic acid molecule comprising the nucleic acid SEQ ID NO. 24232, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 24232 or polypeptide SEQ ID NO. 24233, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 27 to 53-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 38 to 213-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a gibberellin 20-oxidase, or if the activity of the polypeptide At5g07200, preferably represented by SEQ ID NO. 24493, or a homolog or fragment thereof, or if the activity of a polypeptide encoded by a nucleic acid molecule comprising the nucleic acid SEQ ID NO. 24492, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 24492 or polypeptide SEQ ID NO. 24493, respectively, or a homolog or a fragment thereof, is increased or generated, or if the activity gibberellin 20-oxidase is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 28 to 50-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a zinc finger protein, or if the activity of the polypeptide At5g64920, preferably represented by SEQ ID NO. 5494, or a homolog or fragment thereof, or if the activity of a polypeptide encoded by a nucleic acid molecule comprising the nucleic acid SEQ ID NO. 5493, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 5493 or polypeptide SEQ ID NO. 5494, respectively, or a homolog or a fragment thereof, is increased or generated, or if the activity zinc finger protein is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 30 to 57-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a aminotransferase, or if the activity of the polypeptide AvinDRAFT_{—}4562, preferably represented by SEQ ID NO. 31927, or a homolog or fragment thereof, or if the activity of a polypeptide encoded by a nucleic acid molecule comprising the nucleic acid SEQ ID NO. 31926, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 31926 or polypeptide SEQ ID NO. 31927, respectively, or a homolog or a fragment thereof, is increased or generated, or if the activity aminotransferase is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 31 to 40-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a hydrolase, or if the activity of the polypeptide AvinDRAFT_{—}5103, preferably represented by SEQ ID NO. 6511, or a homolog or fragment thereof, or if the activity of a polypeptide encoded by a nucleic acid molecule comprising the nucleic acid SEQ ID NO. 6510, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 6510 or polypeptide SEQ ID NO. 6511, respectively, or a homolog or a fragment thereof, is increased or generated, or if the activity hydrolase is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 35 to 171-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a glutamate-ammonia-ligase, or if the activity of the polypeptide AvinDRAFT_{—}5246, preferably represented by SEQ ID NO. 32038, or a homolog or fragment thereof, or if the activity of a polypeptide encoded by a nucleic acid molecule comprising the nucleic acid SEQ ID NO. 32037, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 32037 or polypeptide SEQ ID NO. 32038, respectively, or a homolog or a fragment thereof, is increased or generated, or if the activity glutamate-ammonia-ligase is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 27 to 137-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a oxidoreductase, or if the activity of the polypeptide AvinDRAFT_{—}5651, preferably represented by SEQ ID NO. 33458, or a homolog or fragment thereof, or if the activity of a polypeptide encoded by a nucleic acid molecule comprising the nucleic acid SEQ ID NO. 33457, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 33457 or polypeptide SEQ ID NO. 33458, respectively, or a homolog or a fragment thereof, is increased or generated, or if the activity oxidoreductase is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 36 to 62-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a chaperone protein CIpB, or if the activity of the polypeptide AvinDRAFT_{—}6093, preferably represented by SEQ ID NO. 33597, or a homolog or fragment thereof, or if the activity of a polypeptide encoded by a nucleic acid molecule comprising the nucleic acid SEQ ID NO. 33596, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 33596 or polypeptide SEQ ID NO. 33597, respectively, or a homolog or a fragment thereof, is increased or generated, or if the activity chaperone protein CIpB is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 32 to 50-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a purine nucleoside phosphorylase, or if the activity of the polypeptide AvinDRAFT_{—}6700, preferably represented by SEQ ID NO. 34045, or a homolog or fragment thereof, or if the activity of a polypeptide encoded by a nucleic acid molecule comprising the nucleic acid SEQ ID NO. 34044, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 34044 or polypeptide SEQ ID NO. 34045, respectively, or a homolog or a fragment thereof, is increased or generated, or if the activity purine nucleoside phosphorylase is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 27 to 99-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a threonine synthase, or if the activity of the polypeptide B0004, preferably represented by SEQ ID NO. 34890, or a homolog or fragment thereof, or if the activity of a polypeptide encoded by a nucleic acid molecule comprising the nucleic acid SEQ ID NO. 34889, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 34889 or polypeptide SEQ ID NO. 34890, respectively, or a homolog or a fragment thereof, is increased or generated, or if the activity threonine synthase is increased or generated in a non-human organism, like a microorganism or a plant cell, plant or part thereof, especially with plastidic localization, whereby the respective line disclose in column 7 the fine chemical lignoceric acid. For example, an increase of the lignoceric acid of at least 1 percent, particularly in a range of 32 to 201-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a serine protease, or if the activity of the polypeptide B0161, preferably represented by SEQ ID NO. 7082, or a homolog or fragment thereof, or if the activity of a polypeptide encoded by a nucleic acid molecule comprising the nucleic acid SEQ ID NO. 7081, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 7081 or polypeptide SEQ ID NO. 7082, respectively, or a homolog or a fragment thereof, is increased or generated, or if the activity serine protease is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 43 to 268-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a ATP-binding component of a transport system, or if the activity of the polypeptide B0449, preferably represented by SEQ ID NO. 7334, or a homolog or fragment thereof, or if the activity of a polypeptide encoded by a nucleic acid molecule comprising the nucleic acid SEQ ID NO. 7333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 7333 or polypeptide SEQ ID NO. 7334, respectively, or a homolog or a fragment thereof, is increased or generated, or if the activity ATP-binding component of a transport system is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 34 to 244-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a isochorismate synthase, or if the activity of the polypeptide B0593, preferably represented by SEQ ID NO. 35968, or a homolog or fragment thereof, or if the activity of a polypeptide encoded by a nucleic acid molecule comprising the nucleic acid SEQ ID NO. 35967, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 35967 or polypeptide SEQ ID NO. 35968, respectively, or a homolog or a fragment thereof, is increased or generated, or if the activity isochorismate synthase is increased or generated in a non-human organism, like a microorganism or a plant cell, plant or part thereof, especially with plastidic localization, whereby the respective line disclose in column 7 the fine chemical lignoceric acid. For example, an increase of the lignoceric acid of at least 1 percent, particularly in a range of 30 to 87-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a leucyl/phenylalanyl-tRNA-protein transferase, or if the activity of the polypeptide B0885, preferably represented by SEQ ID NO. 36490, or a homolog or fragment thereof, or if the activity of a polypeptide encoded by a nucleic acid molecule comprising the nucleic acid SEQ ID NO. 36489, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 36489 or polypeptide SEQ ID NO. 36490, respectively, or a homolog or a fragment thereof, is increased or generated, or if the activity leucyl/phenylalanyl-tRNA-protein transferase is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 38 to 93-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a major facilitator superfamily transporter protein, or if the activity of the polypeptide B0898, preferably represented by SEQ ID NO. 7918, or a homolog or fragment thereof, or if the activity of a polypeptide encoded by a nucleic acid molecule comprising the nucleic acid SEQ ID NO. 7917, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 7917 or polypeptide SEQ ID NO. 7918, respectively, or a homolog or a fragment thereof, is increased or generated, or if the activity major facilitator superfamily transporter protein is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 36 to 337-percent is conferred as compared to a corresponding non-transformed wild type non-human organism.

Accordingly, in one embodiment, an increase of cerotic acid in a non-human organism, as compared to a corresponding non-transformed wild type non-human organism, is conferred in the process of the invention, if the activity of a polypeptide showing the activity of a multidrug resistance protein, or if the activity of the polypeptide B1065, preferably represented by SEQ ID NO. 76033, or a homolog or fragment thereof, or if the activity of a polypeptide encoded by a nucleic acid molecule comprising the nucleic acid SEQ ID NO. 76032, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 76032 or polypeptide SEQ ID NO. 76033, respectively, or a homolog or a fragment thereof, is increased or generated, or if the activity multidrug resistance protein is increased or generated in a non-human organism, like a microorganism or a plant cell, plant or part thereof, especially with plastidic localization, whereby the respective line disclose in column 7 the fine chemical cerotic acid. For example, an increase of the cerotic acid of at least 1 percent, particularly in a range of 41 to 48-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a sodium/proton antiporter, or if the activity of the polypeptide B1186, preferably represented by SEQ ID NO. 37401, or a homolog or fragment thereof, or if the activity of a polypeptide encoded by a nucleic acid molecule comprising the nucleic acid SEQ ID NO. 37400, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 37400 or polypeptide SEQ ID NO. 37401, respectively, or a homolog or a fragment thereof, is increased or generated, or if the activity sodium/proton antiporter is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 35 to 126-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a cardiolipin synthase, or if the activity of the polypeptide B1249, preferably represented by SEQ ID NO. 98779, or a homolog or fragment thereof, or if the activity of a polypeptide encoded by a nucleic acid molecule comprising the nucleic acid SEQ ID NO. 98778, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 98778 or polypeptide SEQ ID NO. 98779, respectively, or a homolog or a fragment thereof, is increased or generated, or if the activity cardiolipin synthase is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 33 to 73-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a b1522-protein, or if the activity of the polypeptide B1522, preferably represented by SEQ ID NO. 7948, or a homolog or fragment thereof, or if the activity of a polypeptide encoded by a nucleic acid molecule comprising the nucleic acid SEQ ID NO. 7947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 7947 or polypeptide SEQ ID NO. 7948, respectively, or a homolog or a fragment thereof, is increased or generated, or if the activity b1522-protein is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 28 to 178-percent is conferred as compared to a corresponding non-transformed wild type non-human organism.

Accordingly, in one embodiment, an increase of cerotic acid in a non-human organism, as compared to a corresponding non-transformed wild type non-human organism, is conferred in the process of the invention, if the activity of a polypeptide showing the activity of a acid shock protein, or if the activity of the polypeptide B1597, preferably represented by SEQ ID NO. 38301, or a homolog or fragment thereof, or if the activity of a polypeptide encoded by a nucleic acid molecule comprising the nucleic acid SEQ ID NO. 38300, preferably the coding region thereof, or a homolog or fragment thereof, e.g. derived from Escherichia coli, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 38300 or polypeptide SEQ ID NO. 38301, respectively, or a homolog or a fragment thereof, is increased or generated, or if the activity acid shock protein is increased or generated in a non-human organism, like a microorganism or a plant cell, plant or part thereof, especially with non-targeted localization, whereby the respective line disclose in column 7 the fine chemical cerotic acid. For example, an increase of the cerotic acid of at least 1 percent, particularly in a range of 43 to 164-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a acid shock protein, or if the activity of the polypeptide B1597, preferably represented by SEQ ID NO. 38301, or a homolog or fragment thereof, or if the activity of a polypeptide encoded by a nucleic acid molecule comprising the nucleic acid SEQ ID NO. 38300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 38300 or polypeptide SEQ ID NO. 38301, respectively, or a homolog or a fragment thereof, is increased or generated, or if the activity acid shock protein is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 35 to 214-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a O-antigen chain length determinant, or if the activity of the polypeptide B2027, preferably represented by SEQ ID NO. 79218, or a homolog or fragment thereof, or if the activity of a polypeptide encoded by a nucleic acid molecule comprising the nucleic acid SEQ ID NO. 79217, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 79217 or polypeptide SEQ ID NO. 79218, respectively, or a homolog or a fragment thereof, is increased or generated, or if the activity O-antigen chain length determinant is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 29 to 85-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a NADH dehydrogenase subunit N, or if the activity of the polypeptide B2482, preferably represented by SEQ ID NO. 79510, or a homolog or fragment thereof, or if the activity of a polypeptide encoded by a nucleic acid molecule comprising the nucleic acid SEQ ID NO. 79509, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 79509 or polypeptide SEQ ID NO. 79510, respectively, or a homolog or a fragment thereof, is increased or generated, or if the activity NADH dehydrogenase subunit N is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 29 to 64-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a 2,3-dihydroxy-2,3-dihydrophenylpropionatedehydrogenase, or if the activity of the polypeptide B2541, preferably represented by SEQ ID NO. 40384, or a homolog or fragment thereof, or if the activity of a polypeptide encoded by a nucleic acid molecule comprising the nucleic acid SEQ ID NO. 40383, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 40383 or polypeptide SEQ ID NO. 40384, respectively, or a homolog or a fragment thereof, is increased or generated, or if the activity 2,3-dihydroxy-2,3-dihydrophenylpropionatedehydrogenase is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 40 to 110-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a b3083-protein, or if the activity of the polypeptide B3083, preferably represented by SEQ ID NO. 82425, or a homolog or fragment thereof, or if the activity of a polypeptide encoded by a nucleic acid molecule comprising the nucleic acid SEQ ID NO. 82424, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 82424 or polypeptide SEQ ID NO. 82425, respectively, or a homolog or a fragment thereof, is increased or generated, or if the activity b3083-protein is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 31 to 63-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a b3812-protein, or if the activity of the polypeptide B3812, preferably represented by SEQ ID NO. 125488, or a homolog or fragment thereof, or if the activity of a polypeptide encoded by a nucleic acid molecule comprising the nucleic acid SEQ ID NO. 125487, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 125487 or polypeptide SEQ ID NO. 125488, respectively, or a homolog or a fragment thereof, is increased or generated, or if the activity b3812-protein is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 35 to 118-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a b4029-protein, or if the activity of the polypeptide B4029, preferably represented by SEQ ID NO. 10741, or a homolog or fragment thereof, or if the activity of a polypeptide encoded by a nucleic acid molecule comprising the nucleic acid SEQ ID NO. 10740, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 10740 or polypeptide SEQ ID NO. 10741, respectively, or a homolog or a fragment thereof, is increased or generated, or if the activity b4029-protein is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 31 to 222-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a acetyltransferase, or if the activity of the polypeptide B4256, preferably represented by SEQ ID NO. 45322, or a homolog or fragment thereof, or if the activity of a polypeptide encoded by a nucleic acid molecule comprising the nucleic acid SEQ ID NO. 45321, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 45321 or polypeptide SEQ ID NO. 45322, respectively, or a homolog or a fragment thereof, is increased or generated, or if the activity acetyltransferase is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 64 to 185-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a glutaredoxin, or if the activity of the polypeptide GM02LC12622, preferably represented by SEQ ID NO. 10812, or a homolog or fragment thereof, or if the activity of a polypeptide encoded by a nucleic acid molecule comprising the nucleic acid SEQ ID NO. 10811, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 10811 or polypeptide SEQ ID NO. 10812, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 28 to 81-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a cyclin D, or if the activity of the polypeptide GM02LC44512, preferably represented by SEQ ID NO. 46851, or a homolog or fragment thereof, or if the activity of a polypeptide encoded by a nucleic acid molecule comprising the nucleic acid SEQ ID NO. 46850, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 46850 or polypeptide SEQ ID NO. 46851, respectively, or a homolog or a fragment thereof, is increased or generated, or if the activity cyclin D is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 30 to 75-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a sll0064-protein, or if the activity of the polypeptide Sll0064, preferably represented by SEQ ID NO. 47527, or a homolog or fragment thereof, or if the activity of a polypeptide encoded by a nucleic acid molecule comprising the nucleic acid SEQ ID NO. 47526, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 47526 or polypeptide SEQ ID NO. 47527, respectively, or a homolog or a fragment thereof, is increased or generated, or if the activity sll0064-protein is increased or generated in a non-human organism, like a microorganism or a plant cell, plant or part thereof, especially with mitochondrial localization, whereby the respective line disclose in column 7 the fine chemical lignoceric acid. For example, an increase of the lignoceric acid of at least 1 percent, particularly in a range of 29 to 56-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a agmatinase, or if the activity of the polypeptide Sll0228, preferably represented by SEQ ID NO. 84266, or a homolog or fragment thereof, or if the activity of a polypeptide encoded by a nucleic acid molecule comprising the nucleic acid SEQ ID NO. 84265, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 84265 or polypeptide SEQ ID NO. 84266, respectively, or a homolog or a fragment thereof, is increased or generated, or if the activity agmatinase is increased or generated in a non-human organism, like a microorganism or a plant cell, plant or part thereof, especially with plastidic localization, whereby the respective line disclose in column 7 the fine chemical lignoceric acid. For example, an increase of the lignoceric acid of at least 1 percent, particularly in a range of 36 to 38-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a uroporphyrin-III C-methyltransferase, or if the activity of the polypeptide Sll0378, preferably represented by SEQ ID NO. 84870, or a homolog or fragment thereof, or if the activity of a polypeptide encoded by a nucleic acid molecule comprising the nucleic acid SEQ ID NO. 84869, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 84869 or polypeptide SEQ ID NO. 84870, respectively, or a homolog or a fragment thereof, is increased or generated, or if the activity uroporphyrin-III C-methyltransferase is increased or generated in a non-human organism, like a microorganism or a plant cell, plant or part thereof, especially with plastidic localization, whereby the respective line disclose in column 7 the fine chemical lignoceric acid. For example, an increase of the lignoceric acid of at least 1 percent, particularly in a range of 32 to 69-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a binding-protein-dependent transport systems inner membrane component, or if the activity of the polypeptide Sll0833, preferably represented by SEQ ID NO. 101708, or a homolog or fragment thereof, or if the activity of a polypeptide encoded by a nucleic acid molecule comprising the nucleic acid SEQ ID NO. 101707, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 101707 or polypeptide SEQ ID NO. 101708, respectively, or a homolog or a fragment thereof, is increased or generated, or if the activity binding-protein-dependent transport systems inner membrane component is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 40 to 112-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a malate dehydrogenase, or if the activity of the polypeptide Sll0891, preferably represented by SEQ ID NO. 51269, or a homolog or fragment thereof, or if the activity of a polypeptide encoded by a nucleic acid molecule comprising the nucleic acid SEQ ID NO. 51268, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 51268 or polypeptide SEQ ID NO. 51269, respectively, or a homolog or a fragment thereof, is increased or generated, or if the activity malate dehydrogenase is increased or generated in a non-human organism, like a microorganism or a plant cell, plant or part thereof, especially with plastidic localization, whereby the respective line disclose in column 7 the fine chemical lignoceric acid. For example, an increase of the lignoceric acid of at least 1 percent, particularly in a range of 32 to 55-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a ABC metal ion transporter substrate-binding protein, or if the activity of the polypeptide Sll1598, preferably represented by SEQ ID NO. 85744, or a homolog or fragment thereof, or if the activity of a polypeptide encoded by a nucleic acid molecule comprising the nucleic acid SEQ ID NO. 85743, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 85743 or polypeptide SEQ ID NO. 85744, respectively, or a homolog or a fragment thereof, is increased or generated, or if the activity ABC metal ion transporter substrate-binding protein is increased or generated in a non-human organism, like a microorganism or a plant cell, plant or part thereof, especially with mitochondrial localization, whereby the respective line disclose in column 7 the fine chemical lignoceric acid. For example, an increase of the lignoceric acid of at least 1 percent, particularly in a range of 27 to 51-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a acyl transferase, or if the activity of the polypeptide Sll1848, preferably represented by SEQ ID NO. 86448, or a homolog or fragment thereof, or if the activity of a polypeptide encoded by a nucleic acid molecule comprising the nucleic acid SEQ ID NO. 86447, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 86447 or polypeptide SEQ ID NO. 86448, respectively, or a homolog or a fragment thereof, is increased or generated, or if the activity acyl transferase is increased or generated in a non-human organism, like a microorganism or a plant cell, plant or part thereof, especially with plastidic localization, whereby the respective line disclose in column 7 the fine chemical lignoceric acid. For example, an increase of the lignoceric acid of at least 1 percent, particularly in a range of 57 to 85-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a slr0383-protein, or if the activity of the polypeptide Slr0383, preferably represented by SEQ ID NO. 95745, or a homolog or fragment thereof, or if the activity of a polypeptide encoded by a nucleic acid molecule comprising the nucleic acid SEQ ID NO. 95744, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 95744 or polypeptide SEQ ID NO. 95745, respectively, or a homolog or a fragment thereof, is increased or generated, or if the activity slr0383-protein is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 32 to 48-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a geranylgeranyl pyrophosphate synthase, or if the activity of the polypeptide Slr0739, preferably represented by SEQ ID NO. 57236, or a homolog or fragment thereof, or if the activity of a polypeptide encoded by a nucleic acid molecule comprising the nucleic acid SEQ ID NO. 57235, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 57235 or polypeptide SEQ ID NO. 57236, respectively, or a homolog or a fragment thereof, is increased or generated, or if the activity geranylgeranyl pyrophosphate synthase is increased or generated in a non-human organism, like a microorganism or a plant cell, plant or part thereof, especially with plastidic localization, whereby the respective line disclose in column 7 the fine chemical lignoceric acid. For example, an increase of the lignoceric acid of at least 1 percent, particularly in a range of 31 to 87-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a tryptophan synthase alpha chain, or if the activity of the polypeptide Slr0966, preferably represented by SEQ ID NO. 87656, or a homolog or fragment thereof, or if the activity of a polypeptide encoded by a nucleic acid molecule comprising the nucleic acid SEQ ID NO. 87655, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 87655 or polypeptide SEQ ID NO. 87656, respectively, or a homolog or a fragment thereof, is increased or generated, or if the activity tryptophan synthase alpha chain is increased or generated in a non-human organism, like a microorganism or a plant cell, plant or part thereof, especially with plastidic localization, whereby the respective line disclose in column 7 the fine chemical lignoceric acid. For example, an increase of the lignoceric acid of at least 1 percent, particularly in a range of 32 to 111-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a rubredoxin, or if the activity of the polypeptide Slr2033, preferably represented by SEQ ID NO. 152276, or a homolog or fragment thereof, or if the activity of a polypeptide encoded by a nucleic acid molecule comprising the nucleic acid SEQ ID NO. 152275, preferably the coding region thereof, or a homolog or fragment thereof, e.g. derived from Synechocystis sp., is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 152275 or polypeptide SEQ ID NO. 152276, respectively, or a homolog or a fragment thereof, is increased or generated, or if the activity rubredoxin is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 28 to 105-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a Sec-independent protein translocase subunit, or if the activity of the polypeptide TT00019, preferably represented by SEQ ID NO. 12699, or a homolog or fragment thereof, or if the activity of a polypeptide encoded by a nucleic acid molecule comprising the nucleic acid SEQ ID NO. 12698, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 12698 or polypeptide SEQ ID NO. 12699, respectively, or a homolog or a fragment thereof, is increased or generated, or if the activity Sec-independent protein translocase subunit is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 44 to 114-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a TT00768-protein, or if the activity of the polypeptide TT00768, preferably represented by SEQ ID NO. 61533, or a homolog or fragment thereof, or if the activity of a polypeptide encoded by a nucleic acid molecule comprising the nucleic acid SEQ ID NO. 61532, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 61532 or polypeptide SEQ ID NO. 61533, respectively, or a homolog or a fragment thereof, is increased or generated, or if the activity TT00768-protein is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 38 to 110-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a TT01386-protein, or if the activity of the polypeptide TT01386, preferably represented by SEQ ID NO. 62080, or a homolog or fragment thereof, or if the activity of a polypeptide encoded by a nucleic acid molecule comprising the nucleic acid SEQ ID NO. 62079, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 62079 or polypeptide SEQ ID NO. 62080, respectively, or a homolog or a fragment thereof, is increased or generated, or if the activity TTC1386-protein is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 49 to 106-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a acetylcoenzyme A synthetase, or if the activity of the polypeptide Yal054c, preferably represented by SEQ ID NO. 121909, or a homolog or fragment thereof, or if the activity of a polypeptide encoded by a nucleic acid molecule comprising the nucleic acid SEQ ID NO. 121908, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 121908 or polypeptide SEQ ID NO. 121909, respectively, or a homolog or a fragment thereof, is increased or generated, or if the activity acetyl-coenzyme A synthetase is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 52 to 58-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a peroxisome assembly protein, or if the activity of the polypeptide Ydr265w, preferably represented by SEQ ID NO. 63666, or a homolog or fragment thereof, or if the activity of a polypeptide encoded by a nucleic acid molecule comprising the nucleic acid SEQ ID NO. 63665, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 63665 or polypeptide SEQ ID NO. 63666, respectively, or a homolog or a fragment thereof, is increased or generated, or if the activity peroxisome assembly protein is increased or generated in a non-human organism, like a microorganism or a plant cell, plant or part thereof, especially with plastidic localization, whereby the respective line disclose in column 7 the fine chemical lignoceric acid. For example, an increase of the lignoceric acid of at least 1 percent, particularly in a range of 28 to 50-percent is conferred as compared to a corresponding non-transformed wild type non-human organism.

Accordingly, in one embodiment, an increase of heneicosanoic acid in a non-human organism, as compared to a corresponding non-transformed wild type non-human organism, is conferred in the process of the invention, if the activity of a polypeptide showing the activity of a inositol-3-phosphate synthase, or if the activity of the polypeptide Yjl153c, preferably represented by SEQ ID NO. 149633, or a homolog or fragment thereof, or if the activity of a polypeptide encoded by a nucleic acid molecule comprising the nucleic acid SEQ ID NO. 149632, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 149632 or polypeptide SEQ ID NO. 149633, respectively, or a homolog or a fragment thereof, is increased or generated, or if the activity inositol-3-phosphate synthase is increased or generated in a non-human organism, like a microorganism or a plant cell, plant or part thereof, especially with non-targeted localization, whereby the respective line disclose in column 7 the fine chemical heneicosanoic acid. For example, an increase of the heneicosanoic acid of at least 1 percent, particularly in a range of 10 to 23-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a cystathionine gamma-synthase, or if the activity of the polypeptide Yjr130c, preferably represented by SEQ ID NO. 14844, or a homolog or fragment thereof, or if the activity of a polypeptide encoded by a nucleic acid molecule comprising the nucleic acid SEQ ID NO. 14843, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 14843 or polypeptide SEQ ID NO. 14844, respectively, or a homolog or a fragment thereof, is increased or generated, or if the activity cystathionine gamma-synthase is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 31 to 42-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a potassium transport protein, or if the activity of the polypeptide Ykr050w, preferably represented by SEQ ID NO. 152383, or a homolog or fragment thereof, or if the activity of a polypeptide encoded by a nucleic acid molecule comprising the nucleic acid SEQ ID NO. 152382, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 152382 or polypeptide SEQ ID NO. 152383, respectively, or a homolog or a fragment thereof, is increased or generated, or if the activity potassium transport protein is increased or generated in a non-human organism, like a microorganism or a plant cell, plant or part thereof, especially with non-targeted localization, whereby the respective line disclose in column 7 the fine chemical lignoceric acid. For example, an increase of the lignoceric acid of at least 1 percent, particularly in a range of 28 to 96-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a ylr065c-protein, or if the activity of the polypeptide Ylr065c, preferably represented by SEQ ID NO. 66696, or a homolog or fragment thereof, or if the activity of a polypeptide encoded by a nucleic acid molecule comprising the nucleic acid SEQ ID NO. 66695, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 66695 or polypeptide SEQ ID NO. 66696, respectively, or a homolog or a fragment thereof, is increased or generated, or if the activity ylr065c-protein is increased or generated in a non-human organism, like a microorganism or a plant cell, plant or part thereof, especially with plastidic localization, whereby the respective line disclose in column 7 the fine chemical lignoceric acid. For example, an increase of the lignoceric acid of at least 1 percent, particularly in a range of 28 to 66-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a yml093w-protein, or if the activity of the polypeptide Yml093w, preferably represented by SEQ ID NO. 152425, or a homolog or fragment thereof, or if the activity of a polypeptide encoded by a nucleic acid molecule comprising the nucleic acid SEQ ID NO. 152424, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 152424 or polypeptide SEQ ID NO. 152425, respectively, or a homolog or a fragment thereof, is increased or generated, or if the activity yml093w-protein is increased or generated in a non-human organism, like a microorganism or a plant cell, plant or part thereof, especially with plastidic localization, whereby the respective line disclose in column 7 the fine chemical lignoceric acid. For example, an increase of the lignoceric acid of at least 1 percent, particularly in a range of 34 to 69 is conferred as compared to a corresponding non-transformed wild type non-human-percent organism.

Accordingly, in one embodiment, an increase of lignoceric acid in a non-human organism, as compared to a corresponding non-transformed wild type non-human organism, is conferred in the process of the invention, if the activity of a polypeptide showing the activity of a mitochondrial inner membrane protease subunit, or if the activity of the polypeptide Ymr150c, preferably represented by SEQ ID NO. 92275, or a homolog or fragment thereof, or if the activity of a polypeptide encoded by a nucleic acid molecule comprising the nucleic acid SEQ ID NO. 92274, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 92274 or polypeptide SEQ ID NO. 92275, respectively, or a homolog or a fragment thereof, is increased or generated, or if the activity mitochondrial inner membrane protease subunit is increased or generated in a non-human organism, like a microorganism or a plant cell, plant or part thereof, especially with plastidic localization, whereby the respective line disclose in column 7 the fine chemical lignoceric acid. For example, an increase of the lignoceric acid of at least 1 percent, particularly in a range of 29 to 74-percent is conferred as compared to a corresponding non-transformed wild type non-human organism.

Accordingly, in one embodiment, an increase of lignoceric acid in a non-human organism, as compared to a corresponding non-transformed wild type non-human organism, is conferred in the process of the invention, if the activity of a polypeptide showing the activity of a mevalonate kinase, or if the activity of the polypeptide Ymr208w, preferably represented by SEQ ID NO. 152461, or a homolog or fragment thereof, or if the activity of a polypeptide encoded by a nucleic acid molecule comprising the nucleic acid SEQ ID NO. 152460, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 152460 or polypeptide SEQ ID NO. 152461, respectively, or a homolog or a fragment thereof, is increased or generated, or if the activity mevalonate kinase is increased or generated in a non-human organism, like a microorganism or a plant cell, plant or part thereof, especially with plastidic localization, whereby the respective line disclose in column 7 the fine chemical lignoceric acid. For example, an increase of the lignoceric acid of at least 1 percent, particularly in a range of 30 to 44-percent is conferred as compared to a corresponding non-transformed wild type non-human organism.

Accordingly, in one embodiment, an increase of melissic acid in a non-human organism, as compared to a corresponding non-transformed wild type non-human organism, is conferred in the process of the invention, if the activity of a polypeptide showing the activity of a yer152c-protein, or if the activity of the polypeptide Yer152c, preferably represented by SEQ ID NO. 152342, or a homolog or fragment thereof, or if the activity of a polypeptide encoded by a nucleic acid molecule comprising the nucleic acid SEQ ID NO. 152341, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 152341 or polypeptide SEQ ID NO. 152342, respectively, or a homolog or a fragment thereof, is increased or generated, or if the activity yer152c-protein is increased or generated in a non-human organism, like a microorganism or a plant cell, plant or part thereof, especially with non-targeted localization, whereby the respective line disclose in column 7 the fine chemical melissic acid. For example, an increase of the melissic acid of at least 1 percent, particularly in a range of 1 to 69-percent is conferred as compared to a corresponding non-transformed wild type non-human organism.

Accordingly, in one embodiment, an increase of montanic acid in a non-human organism, as compared to a corresponding non-transformed wild type non-human organism, is conferred in the process of the invention, if the activity of a polypeptide showing the activity of a yer152c-protein, or if the activity of the polypeptide Yer152c, preferably represented by SEQ ID NO. 152342, or a homolog or fragment thereof, or if the activity of a polypeptide encoded by a nucleic acid molecule comprising the nucleic acid SEQ ID NO. 152341, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 152341 or polypeptide SEQ ID NO. 152342, respectively, or a homolog or a fragment thereof, is increased or generated, or if the activity yer152c-protein is increased or generated in a non-human organism, like a microorganism or a plant cell, plant or part thereof, especially with non-targeted localization, whereby the respective line disclose in column 7 the fine chemical montanic acid. For example, an increase of the montanic acid of at least 1 percent, particularly in a range of 3 to 93-percent is conferred as compared to a corresponding non-transformed wild type non-human organism.

Accordingly, in one embodiment, an increase of melissic acid in a non-human organism, as compared to a corresponding non-transformed wild type non-human organism, is conferred in the process of the invention, if the activity of a polypeptide showing the activity of a b3644-protein, or if the activity of the polypeptide B3644, preferably represented by SEQ ID NO. 125349, or a homolog or fragment thereof, or if the activity of a polypeptide encoded by a nuoleic acid molecule comprising the nucleic acid SEQ ID NO. 125348, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 125348 or polypeptide SEQ ID NO. 125349, respectively, or a homolog or a fragment thereof, is increased or generated, or if the activity b3644-protein is increased or generated in a non-human organism, like a microorganism or a plant cell, plant or part thereof, especially with non-targeted localization, whereby the respective line disclose in column 7 the fine chemical melissic acid. For example, an increase of the melissic acid of at least 1 percent, particularly in a range of 3 to 56-percent is conferred as compared to a corresponding non-transformed wild type non-human organism.

Accordingly, in one embodiment, an increase of montanic acid in a non-human organism, as compared to a corresponding non-transformed wild type non-human organism, is conferred in the process of the invention, if the activity of a polypeptide showing the activity of a b3644-protein, or if the activity of the polypeptide B3644, preferably represented by SEQ ID NO. 125349, or a homolog or fragment thereof, or if the activity of a polypeptide encoded by a nucleic acid molecule comprising the nucleic acid SEQ ID NO. 125348, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 18, column 5 or 8 in the respective same line as the nucleic acid molecule SEQ ID NO. 125348 or polypeptide SEQ ID NO. 125349, respectively, or a homolog or a fragment thereof, is increased or generated, or if the activity b3644-protein is increased or generated in a non-human organism, like a microorganism or a plant cell, plant or part thereof, especially with non-targeted localization, whereby the respective line disclose in column 7 the fine chemical montanic acid. For example, an increase of the montanic acid of at least 1 percent, particularly in a range of 5 to 49-percent is conferred as compared to a corresponding non-transformed wild type non-human organism.

[0096.1.7.18] to [0103.1.7.18] for the disclosure of these paragraphs see [0096.1.7.7] to [0103.1.7.7] above

In this paragraph “A” means “Goffeau et al., Science 274 (5287), 546 (1996)”, “B” means “Blattner et al., Science 277 (5331), 1453 (1997)”, “C” means “Nakamura et al., Nucleic Acids Res. 28(1), 72 (2000)”, “D” means “deposition of the sequences to the EMBL/GenBank/DDBJ databases under “Sequencing of the draft genome assembly of Azotobacter vinelandii AvOP”. in June 2005 by Copeland et al.”, “E” means “Henne et al., Nat. Biotechnol. 22 (5), 547 (2004)”, “F” means “Kaul et al., Nature 408 (6814), 796 (2000)”, and “G” means “Matsumoto et al., Nature 436 (7052), 793 (2005)”.

The nucleic acid sequence of At1g36730 from Arabidopsis thaliana, e.g. as shown in the respective line in column 5 of Table I, application no. 18, has been published in F. And the activity of the gene product thereof is the activity of eukaryotic translation initiation factor 5.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “eukaryotic translation initiation factor 5”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said At1g36730, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said At1g36730, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said At1g36730, or a functional equivalent or a homolog thereof as depicted in column 8 of Table II, application no. 18, preferably a homolog or functional equivalent as depicted in column 8 of Table II B, application no. 18, and being depicted in the same respective line as said At1g36730, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “eukaryotic translation initiation factor 5”, preferably it is the molecule of section (a) or (b) of this paragraph.

In particular, it was observed that in plants, especially in Arabidopsis thaliana, increasing or generating the activity of a gene product non-targeted with the activity of a “eukaryotic translation initiation factor 5”, preferably being encoded by a gene comprising the nucleic acid sequence SEQ ID NO. 17968, preferably the coding region thereof, conferred the production of or the increase in lignoceric acid compared with the wild type control.

The nucleic acid sequence of At1g48260 from Arabidopsis thaliana, e.g. as shown in the respective line in column 5 of Table I, application no. 18, has been published in F. And the activity of the gene product thereof is the activity of protein kinase.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said At1g48260, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said At1g48260, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said At1g48260, or a functional equivalent or a homolog thereof as depicted in column 8 of Table II, application no. 18, preferably a homolog or functional equivalent as depicted in column 8 of Table II B, application no. 18, and being depicted in the same respective line as said At1g48260, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “protein kinase”, preferably it is the molecule of section (a) or (b) of this paragraph.

In particular, it was observed that in plants, especially in Arabidopsis thaliana, increasing or generating the activity of a gene product non-targeted with the activity of a “protein kinase”, preferably being encoded by a gene comprising the nucleic acid sequence SEQ ID NO. 18235, preferably the coding region thereof, conferred the production of or the increase in lignoceric acid compared with the wild type control.

The nucleic acid sequence of At3g08710 from Arabidopsis thaliana, e.g. as shown in the respective line in column 5 of Table I, application no. 18, has been published in F. And the activity of the gene product thereof is the activity of thioredoxin family protein.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “thioredoxin family protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said At3g08710, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said At3g08710, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said At3g08710, or a functional equivalent or a homolog thereof as depicted in column 8 of Table II, application no. 18, preferably a homolog or functional equivalent as depicted in column 8 of Table II B, application no. 18, and being depicted in the same respective line as said At3g08710, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “thioredoxin family protein”, preferably it is the molecule of section (a) or (b) of this paragraph.

In particular, it was observed that in plants, especially in Arabidopsis thaliana, increasing or generating the activity of a gene product non-targeted with the activity of a “thioredoxin family protein”, preferably being encoded by a gene comprising the nucleic acid sequence SEQ ID NO. 22249, preferably the coding region thereof, conferred the production of or the increase in lignoceric acid compared with the wild type control.

The nucleic acid sequence of At3g09940 from Arabidopsis thaliana, e.g. as shown in the respective line in column 5 of Table I, application no. 18, has been published in F. And the activity of the gene product thereof is the activity of monodehydroascorbate reductase.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “monodehydroascorbate reductase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said At3g09940, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said At3g09940, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said At3g09940, or a functional equivalent or a homolog thereof as depicted in column 8 of Table II, application no. 18, preferably a homolog or functional equivalent as depicted in column 8 of Table II B, application no. 18, and being depicted in the same respective line as said At3g09940, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “monodehydroascorbate reductase”, preferably it is the molecule of section (a) or (b) of this paragraph.

In particular, it was observed that in plants, especially in Arabidopsis thaliana, increasing or generating the activity of a gene product non-targeted with the activity of a “monodehydroascorbate reductase”, preferably being encoded by a gene comprising the nucleic acid sequence SEQ ID NO. 152081, preferably the coding region thereof, conferred the production of or the increase in lignoceric acid compared with the wild type control.

The nucleic acid sequence of At3g27540 from Arabidopsis thaliana, e.g. as shown in the respective line in column 5 of Table I, application no. 18, has been published in F. And the activity of the gene product thereof is the activity of glycosyl transferase.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “glycosyl transferase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said At3g27540, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said At3g27540, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said At3g27540, or a functional equivalent or a homolog thereof as depicted in column 8 of Table II, application no. 18, preferably a homolog or functional equivalent as depicted in column 8 of Table II B, application no. 18, and being depicted in the same respective line as said At3g27540, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “glycosyl transferase”, preferably it is the molecule of section (a) or (b) of this paragraph.

In particular, it was observed that in plants, especially in Arabidopsis thaliana, increasing or generating the activity of a gene product non-targeted with the activity of a “glycosyl transferase”, preferably being encoded by a gene comprising the nucleic acid sequence SEQ ID NO. 68777, preferably the coding region thereof, conferred the production of or the increase in lignoceric acid compared with the wild type control.

The nucleic acid sequence of At3g62950 from Arabidopsis thaliana, e.g. as shown in the respective line in column 5 of Table I, application no. 18, has been published in F. And the activity of the gene product thereof is the activity of glutaredoxin.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said At3g62950, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said At3g62950, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said At3g62950, or a functional equivalent or a homolog thereof as depicted in column 8 of Table II, application no. 18, preferably a homolog or functional equivalent as depicted in column 8 of Table II B, application no. 18, and being depicted in the same respective line as said At3g62950, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 2935, preferably the coding region thereof, conferred the production of or the increase in lignoceric acid compared with the wild type control.

The nucleic acid sequence of At4g18880 from Arabidopsis thaliana, e.g. as shown in the respective line in column 5 of Table I, application no. 18, has been published in F. And the activity of the gene product thereof is the activity of heat shock transcription factor.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said At4g18880, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said At4g18880, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said At4g18880, or a functional equivalent or a homolog thereof as depicted in column 8 of Table II, application no. 18, preferably a homolog or functional equivalent as depicted in column 8 of Table II B, application no. 18, and being depicted in the same respective line as said At4g18880, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 24232, preferably the coding region thereof, conferred the production of or the increase in lignoceric acid compared with the wild type control.

The nucleic acid sequence of At4g35310 from Arabidopsis thaliana, e.g. as shown in the respective line in column 5 of Table I, application no. 18, has been published in F. And the activity of the gene product thereof is the activity of calcium-dependent protein kinase.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said At4g35310, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said At4g35310, or a functional equivalent or a homolog thereof as depicted in column 8 of Table II, application no. 18, preferably a homolog or functional equivalent as depicted in column 8 of Table II B, application no. 18, and being depicted in the same respective line as said At4g35310, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in lignoceric acid compared with the wild type control.

The nucleic acid sequence of At5g07200 from Arabidopsis thaliana, e.g. as shown in the respective line in column 5 of Table I, application no. 18, has been published in F. And the activity of the gene product thereof is the activity of gibberellin 20-oxidase.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “gibberellin 20-oxidase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said At5g07200, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said At5g07200, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said At5g07200, or a functional equivalent or a homolog thereof as depicted in column 8 of Table II, application no. 18, preferably a homolog or functional equivalent as depicted in column 8 of Table II B, application no. 18, and being depicted in the same respective line as said At5g07200, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “gibberellin 20-oxidase”, preferably it is the molecule of section (a) or (b) of this paragraph.

In particular, it was observed that in plants, especially in Arabidopsis thaliana, increasing or generating the activity of a gene product non-targeted with the activity of a “gibberellin 20-oxidase”, preferably being encoded by a gene comprising the nucleic acid sequence SEQ ID NO. 24492, preferably the coding region thereof, conferred the production of or the increase in lignoceric acid compared with the wild type control.

The nucleic acid sequence of At5g64920 from Arabidopsis thaliana, e.g. as shown in the respective line in column 5 of Table I, application no. 18, has been published in F. And the activity of the gene product thereof is the activity of zinc finger protein.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “zinc finger protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said At5g64920, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said At5g64920, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said At5g64920, or a functional equivalent or a homolog thereof as depicted in column 8 of Table II, application no. 18, preferably a homolog or functional equivalent as depicted in column 8 of Table II B, application no. 18, and being depicted in the same respective line as said At5g64920, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “zinc finger protein”, preferably it is the molecule of section (a) or (b) of this paragraph.

In particular, it was observed that in plants, especially in Arabidopsis thaliana, increasing or generating the activity of a gene product non-targeted with the activity of a “zinc finger protein”, preferably being encoded by a gene comprising the nucleic acid sequence SEQ ID NO. 5493, preferably the coding region thereof, conferred the production of or the increase in lignoceric acid compared with the wild type control.

The nucleic acid sequence of AvinDRAFT_{—}4562 from Azotobacter vinelandii, e.g. as shown in the respective line in column 5 of Table I, application no. 18, has been published in D. And the activity of the gene product thereof is the activity of aminotransferase.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “aminotransferase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said AvinDRAFT_{—}4562, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said AvinDRAFT_{—}4562, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said AvinDRAFT_{—}4562, or a functional equivalent or a homolog thereof as depicted in column 8 of Table II, application no. 18, preferably a homolog or functional equivalent as depicted in column 8 of Table II B, application no. 18, and being depicted in the same respective line as said AvinDRAFT_{—}4562, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “aminotransferase”, preferably it is the molecule of section (a) or (b) of this paragraph.

In particular, it was observed that in plants, especially in Arabidopsis thaliana, increasing or generating the activity of a gene product non-targeted with the activity of a “aminotransferase”, preferably being encoded by a gene comprising the nucleic acid sequence SEQ ID NO. 31926, preferably the coding region thereof, conferred the production of or the increase in lignoceric acid compared with the wild type control.

The nucleic acid sequence of AvinDRAFT_{—}5103 from Azotobacter vinelandii, e.g. as shown in the respective line in column 5 of Table I, application no. 18, has been published in D. And the activity of the gene product thereof is the activity of hydrolase.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “hydrolase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said AvinDRAFT_{—}5103, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said AvinDRAFT_{—}5103, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said AvinDRAFT_{—}5103, or a functional equivalent or a homolog thereof as depicted in column 8 of Table II, application no. 18, preferably a homolog or functional equivalent as depicted in column 8 of Table II B, application no. 18, and being depicted in the same respective line as said AvinDRAFT_{—}5103, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “hydrolase”, preferably it is the molecule of section (a) or (b) of this paragraph.

In particular, it was observed that in plants, especially in Arabidopsis thaliana, increasing or generating the activity of a gene product non-targeted with the activity of a “hydrolase”, preferably being encoded by a gene comprising the nucleic acid sequence SEQ ID NO. 6510, preferably the coding region thereof, conferred the production of or the increase in lignoceric acid compared with the wild type control.

The nucleic acid sequence of AvinDRAFT_{—}5246 from Azotobacter vinelandii, e.g. as shown in the respective line in column 5 of Table I, application no. 18, has been published in D. And the activity of the gene product thereof is the activity of glutamate-ammonia-ligase.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “glutamate-ammonia-ligase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said AvinDRAFT_{—}5246, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said AvinDRAFT_{—}5246, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said AvinDRAFT_{—}5246, or a functional equivalent or a homolog thereof as depicted in column 8 of Table II, application no. 18, preferably a homolog or functional equivalent as depicted in column 8 of Table II B, application no. 18, and being depicted in the same respective line as said AvinDRAFT_{—}5246, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “glutamate-ammonia-ligase”, preferably it is the molecule of section (a) or (b) of this paragraph.

In particular, it was observed that in plants, especially in Arabidopsis thaliana, increasing or generating the activity of a gene product non-targeted with the activity of a “glutamate-ammonia-ligase”, preferably being encoded by a gene comprising the nucleic acid sequence SEQ ID NO. 32037, preferably the coding region thereof, conferred the production of or the increase in lignoceric acid compared with the wild type control.

The nucleic acid sequence of AvinDRAFT_{—}5651 from Azotobacter vinelandii, e.g. as shown in the respective line in column 5 of Table I, application no. 18, has been published in D. And the activity of the gene product thereof is the activity of oxidoreductase.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “oxidoreductase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said AvinDRAFT_{—}5651, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said AvinDRAFT_{—}5651, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said AvinDRAFT_{—}5651, or a functional equivalent or a homolog thereof as depicted in column 8 of Table II, application no. 18, preferably a homolog or functional equivalent as depicted in column 8 of Table II B, application no. 18, and being depicted in the same respective line as said AvinDRAFT_{—}5651, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 33457, preferably the coding region thereof, conferred the production of or the increase in lignoceric acid compared with the wild type control.

The nucleic acid sequence of AvinDRAFT_{—}6093 from Azotobacter vinelandii, e.g. as shown in the respective line in column 5 of Table I, application no. 18, has been published in D. And the activity of the gene product thereof is the activity of chaperone protein CIpB.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “chaperone protein CIpB”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said AvinDRAFT_{—}6093, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said AvinDRAFT_{—}6093, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said AvinDRAFT_{—}6093, or a functional equivalent or a homolog thereof as depicted in column 8 of Table II, application no. 18, preferably a homolog or functional equivalent as depicted in column 8 of Table II B, application no. 18, and being depicted in the same respective line as said AvinDRAFT_{—}6093, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “chaperone protein CIpB”, preferably it is the molecule of section (a) or (b) of this paragraph.

In particular, it was observed that in plants, especially in Arabidopsis thaliana, increasing or generating the activity of a gene product non-targeted with the activity of a “chaperone protein CIpB”, preferably being encoded by a gene comprising the nucleic acid sequence SEQ ID NO. 33596, preferably the coding region thereof, conferred the production of or the increase in lignoceric acid compared with the wild type control.

The nucleic acid sequence of AvinDRAFT_{—}6700 from Azotobacter vinelandii, e.g. as shown in the respective line in column 5 of Table I, application no. 18, has been published in D. And the activity of the gene product thereof is the activity of purine nucleoside phosphorylase.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “purine nucleoside phosphorylase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said AvinDRAFT_{—}6700, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said AvinDRAFT_{—}6700, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said AvinDRAFT_{—}6700, or a functional equivalent or a homolog thereof as depicted in column 8 of Table II, application no. 18, preferably a homolog or functional equivalent as depicted in column 8 of Table II B, application no. 18, and being depicted in the same respective line as said AvinDRAFT_{—}6700, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “purine nucleoside phosphorylase”, preferably it is the molecule of section (a) or (b) of this paragraph.

In particular, it was observed that in plants, especially in Arabidopsis thaliana, increasing or generating the activity of a gene product non-targeted with the activity of a “purine nucleoside phosphorylase”, preferably being encoded by a gene comprising the nucleic acid sequence SEQ ID NO. 34044, preferably the coding region thereof, conferred the production of or the increase in lignoceric acid compared with the wild type control.

The nucleic acid sequence of B0004 from Escherichia coli, e.g. as shown in the respective line in column 5 of Table I, application no. 18, has been published in B. And the activity of the gene product thereof is the activity of threonine synthase.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “threonine synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said B0004, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said B0004, and preferably the activity is increased plastidic, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said B0004, or a functional equivalent or a homolog thereof as depicted in column 8 of Table II, application no. 18, preferably a homolog or functional equivalent as depicted in column 8 of Table II B, application no. 18, and being depicted in the same respective line as said B0004, and preferably the activity is increased plastidic, whereby the respective line disclose in column 7 the fine chemical lignoceric acid.

Accordingly, in one embodiment, the molecule which activity is to be increased in the process of the invention is the gene product with an activity as a “threonine synthase”, preferably it is the molecule of section (a) or (b) of this paragraph.

In particular, it was observed that in plants, especially in Arabidopsis thaliana, increasing or generating the activity of a gene product plastidic with the activity of a “threonine synthase”, preferably being encoded by a gene comprising the nucleic acid sequence SEQ ID NO. 34889, preferably the coding region thereof, conferred the production of or the increase in lignoceric acid compared with the wild type control.

The nucleic acid sequence of B0161 from Escherichia coli, e.g. as shown in the respective line in column 5 of Table I, application no. 18, has been published in B. And the activity of the gene product thereof is the activity of serine protease.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “serine protease”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said B0161, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said B0161, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said B0161, or a functional equivalent or a homolog thereof as depicted in column 8 of Table II, application no. 18, preferably a homolog or functional equivalent as depicted in column 8 of Table II B, application no. 18, and being depicted in the same respective line as said B0161, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “serine protease”, preferably it is the molecule of section (a) or (b) of this paragraph.

In particular, it was observed that in plants, especially in Arabidopsis thaliana, increasing or generating the activity of a gene product non-targeted with the activity of a “serine protease”, preferably being encoded by a gene comprising the nucleic acid sequence SEQ ID NO. 7081, preferably the coding region thereof, conferred the production of or the increase in lignoceric acid compared with the wild type control.

The nucleic acid sequence of B0449 from Escherichia coli, e.g. as shown in the respective line in column 5 of Table I, application no. 18, has been published in B. And the activity of the gene product thereof is the activity of ATP-binding component of a transport system.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “ATP-binding component of a transport system”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said B0449, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said B0449, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said B0449, or a functional equivalent or a homolog thereof as depicted in column 8 of Table II, application no. 18, preferably a homolog or functional equivalent as depicted in column 8 of Table II B, application no. 18, and being depicted in the same respective line as said B0449, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “ATP-binding component of a transport system”, preferably it is the molecule of section (a) or (b) of this paragraph.

In particular, it was observed that in plants, especially in Arabidopsis thaliana, increasing or generating the activity of a gene product non-targeted with the activity of a “ATP-binding component of a transport system”, preferably being encoded by a gene comprising the nucleic acid sequence SEQ ID NO. 7333, preferably the coding region thereof, conferred the production of or the increase in lignoceric acid compared with the wild type control.

The nucleic acid sequence of B0593 from Escherichia coli, e.g. as shown in the respective line in column 5 of Table I, application no. 18, has been published in B. And the activity of the gene product thereof is the activity of isochorismate synthase.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “isochorismate synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said B0593, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said B0593, and preferably the activity is increased plastidic, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said B0593, or a functional equivalent or a homolog thereof as depicted in column 8 of Table II, application no. 18, preferably a homolog or functional equivalent as depicted in column 8 of Table II B, application no. 18, and being depicted in the same respective line as said B0593, and preferably the activity is increased plastidic, whereby the respective line disclose in column 7 the fine chemical lignoceric acid.

Accordingly, in one embodiment, the molecule which activity is to be increased in the process of the invention is the gene product with an activity as a “isochorismate synthase”, preferably it is the molecule of section (a) or (b) of this paragraph.

In particular, it was observed that in plants, especially in Arabidopsis thaliana, increasing or generating the activity of a gene product plastidic with the activity of a “isochorismate synthase”, preferably being encoded by a gene comprising the nucleic acid sequence SEQ ID NO. 35967, preferably the coding region thereof, conferred the production of or the increase in lignoceric acid compared with the wild type control.

The nucleic acid sequence of B0885 from Escherichia coli, e.g. as shown in the respective line in column 5 of Table I, application no. 18, has been published in B. And the activity of the gene product thereof is the activity of leucyl/phenylalanyl-tRNA-protein transferase.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “leucyl/phenylalanyl-tRNA-protein transferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said B0885, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said B0885, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said B0885, or a functional equivalent or a homolog thereof as depicted in column 8 of Table II, application no. 18, preferably a homolog or functional equivalent as depicted in column 8 of Table II B, application no. 18, and being depicted in the same respective line as said B0885, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “leucyl/phenylalanyl-tRNA-protein transferase”, preferably it is the molecule of section (a) or (b) of this paragraph.

In particular, it was observed that in plants, especially in Arabidopsis thaliana, increasing or generating the activity of a gene product non-targeted with the activity of a “leucyl/phenylalanyltRNA-protein transferase”, preferably being encoded by a gene comprising the nucleic acid sequence SEQ ID NO. 36489, preferably the coding region thereof, conferred the production of or the increase in lignoceric acid compared with the wild type control.

The nucleic acid sequence of B0898 from Escherichia coli, e.g. as shown in the respective line in column 5 of Table I, application no. 18, has been published in B. And the activity of the gene product thereof is the activity of major facilitator superfamily transporter protein.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “major facilitator superfamily transporter protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said B0898, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said B0898, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said B0898, or a functional equivalent or a homolog thereof as depicted in column 8 of Table II, application no. 18, preferably a homolog or functional equivalent as depicted in column 8 of Table II B, application no. 18, and being depicted in the same respective line as said B0898, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “major facilitator superfamily transporter protein”, preferably it is the molecule of section (a) or (b) of this paragraph.

In particular, it was observed that in plants, especially in Arabidopsis thaliana, increasing or generating the activity of a gene product non-targeted with the activity of a “major facilitator superfamily transporter protein”, preferably being encoded by a gene comprising the nucleic acid sequence SEQ ID NO. 7917, preferably the coding region thereof, conferred the production of or the increase in lignoceric acid compared with the wild type control.

The nucleic acid sequence of B1065 from Escherichia coli, e.g. as shown in the respective line in column 5 of Table I, application no. 18, has been published in B. And the activity of the gene product thereof is the activity of multidrug resistance protein.

Accordingly, in one embodiment, the process of the present invention for producing cerotic acid in a non-human organism, like a microorganism or a plant or a part thereof, comprises increasing or generating the activity of a gene product with the activity of a gene product conferring the activity of “multidrug resistance protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical cerotic acid), application no. 18, preferably the coding region thereof, or a homolog or a fragment thereof, and being depicted in the same respective line as said B1065, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said B1065, and preferably the activity is increased plastidic, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said B1065, or a functional equivalent or a homolog thereof as depicted in column 8 of Table II, application no. 18, preferably a homolog or functional equivalent as depicted in column 8 of Table II B, application no. 18, and being depicted in the same respective line as said B1065, and preferably the activity is increased plastidic, whereby the respective line disclose in column 7 the fine chemical cerotic acid.

Accordingly, in one embodiment, the molecule which activity is to be increased in the process of the invention is the gene product with an activity as a “multidrug resistance protein”, preferably it is the molecule of section (a) or (b) of this paragraph.

In particular, it was observed that in plants, especially in Arabidopsis thaliana, increasing or generating the activity of a gene product plastidic with the activity of a “multidrug resistance protein”, preferably being encoded by a gene comprising the nucleic acid sequence SEQ ID NO. 76032, preferably the coding region thereof, conferred the production of or the increase in cerotic acid compared with the wild type control.

The nucleic acid sequence of B1186 from Escherichia coli, e.g. as shown in the respective line in column 5 of Table I, application no. 18, has been published in B. And the activity of the gene product thereof is the activity of sodium/proton antiporter.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “sodium/proton antiporter”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said B1186, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said B1186, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said B1186, or a functional equivalent or a homolog thereof as depicted in column 8 of Table II, application no. 18, preferably a homolog or functional equivalent as depicted in column 8 of Table II B, application no. 18, and being depicted in the same respective line as said B1186, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “sodium/proton antiporter”, preferably it is the molecule of section (a) or (b) of this paragraph.

In particular, it was observed that in plants, especially in Arabidopsis thaliana, increasing or generating the activity of a gene product non-targeted with the activity of a “sodium/proton antiporter”, preferably being encoded by a gene comprising the nucleic acid sequence SEQ ID NO. 37400, preferably the coding region thereof, conferred the production of or the increase in lignoceric acid compared with the wild type control.

The nucleic acid sequence of B1249 from Escherichia coli, e.g. as shown in the respective line in column 5 of Table I, application no. 18, has been published in B. And the activity of the gene product thereof is the activity of cardiolipin synthase.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “cardiolipin synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said B1249, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said B1249, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said B1249, or a functional equivalent or a homolog thereof as depicted in column 8 of Table II, application no. 18, preferably a homolog or functional equivalent as depicted in column 8 of Table II B, application no. 18, and being depicted in the same respective line as said B1249, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “cardiolipin synthase”, preferably it is the molecule of section (a) or (b) of this paragraph.

In particular, it was observed that in plants, especially in Arabidopsis thaliana, increasing or generating the activity of a gene product non-targeted with the activity of a “cardiolipin synthase”, preferably being encoded by a gene comprising the nucleic acid sequence SEQ ID NO. 98778, preferably the coding region thereof, conferred the production of or the increase in lignoceric acid compared with the wild type control.

The nucleic acid sequence of B1522 from Escherichia coli, e.g. as shown in the respective line in column 5 of Table I, application no. 18, has been published in B. And the activity of the gene product thereof is the activity of b1522-protein.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “b1522-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said B1522, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said B1522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said B1522, or a functional equivalent or a homolog thereof as depicted in column 8 of Table II, application no. 18, preferably a homolog or functional equivalent as depicted in column 8 of Table II B, application no. 18, and being depicted in the same respective line as said B1522, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “b1522-protein”, preferably it is the molecule of section (a) or (b) of this paragraph.

In particular, it was observed that in plants, especially in Arabidopsis thaliana, increasing or generating the activity of a gene product non-targeted with the activity of a “b1522-protein”, preferably being encoded by a gene comprising the nucleic acid sequence SEQ ID NO. 7947, preferably the coding region thereof, conferred the production of or the increase in lignoceric acid compared with the wild type control.

The nucleic acid sequence of B1597 from Escherichia coli, e.g. as shown in the respective line in column 5 of Table I, application no. 18, has been published in B. And the activity of the gene product thereof is the activity of acid shock protein.

Accordingly, in one embodiment, the process of the present invention for producing cerotic acid in a non-human organism, like a microorganism or a plant or a part thereof, comprises increasing or generating the activity of a gene product with the activity of a gene product conferring the activity of “acid shock protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical cerotic acid), application no. 18, preferably the coding region thereof, or a homolog or a fragment thereof, and being depicted in the same respective line as said B1597, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said B1597, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said B1597, or a functional equivalent or a homolog thereof as depicted in column 8 of Table II, application no. 18, preferably a homolog or functional equivalent as depicted in column 8 of Table II B, application no. 18, and being depicted in the same respective line as said B1597, and preferably the activity is increased non-targeted, whereby the respective line disclose in column 7 the fine chemical cerotic acid.

Accordingly, in one embodiment, the molecule which activity is to be increased in the process of the invention is the gene product with an activity as a “acid shock protein”, preferably it is the molecule of section (a) or (b) of this paragraph.

In particular, it was observed that in plants, especially in Arabidopsis thaliana, increasing or generating the activity of a gene product non-targeted with the activity of a “acid shock protein”, preferably being encoded by a gene comprising the nucleic acid sequence SEQ ID NO. 38300, preferably the coding region thereof, conferred the production of or the increase in cerotic acid compared with the wild type control.

The nucleic acid sequence of B1597 from Escherichia coli, e.g. as shown in the respective line in column 5 of Table I, application no. 18, has been published in B. And the activity of the gene product thereof is the activity of acid shock protein.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “acid shock protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said B1597, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said B1597, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said B1597, or a functional equivalent or a homolog thereof as depicted in column 8 of Table II, application no. 18, preferably a homolog or functional equivalent as depicted in column 8 of Table II B, application no. 18, and being depicted in the same respective line as said B1597, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “acid shock protein”, preferably it is the molecule of section (a) or (b) of this paragraph.

In particular, it was observed that in plants, especially in Arabidopsis thaliana, increasing or generating the activity of a gene product non-targeted with the activity of a “acid shock protein”, preferably being encoded by a gene comprising the nucleic acid sequence SEQ ID NO. 38300, preferably the coding region thereof, conferred the production of or the increase in lignoceric acid compared with the wild type control.

The nucleic acid sequence of B2027 from Escherichia coli, e.g. as shown in the respective line in column 5 of Table I, application no. 18, has been published in B. And the activity of the gene product thereof is the activity of O-antigen chain length determinant.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “O-antigen chain length determinant”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said B2027, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said B2027, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said B2027, or a functional equivalent or a homolog thereof as depicted in column 8 of Table II, application no. 18, preferably a homolog or functional equivalent as depicted in column 8 of Table II B, application no. 18, and being depicted in the same respective line as said B2027, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “O-antigen chain length determinant”, preferably it is the molecule of section (a) or (b) of this paragraph.

In particular, it was observed that in plants, especially in Arabidopsis thaliana, increasing or generating the activity of a gene product non-targeted with the activity of a “O-antigen chain length determinant”, preferably being encoded by a gene comprising the nucleic acid sequence SEQ ID NO. 79217, preferably the coding region thereof, conferred the production of or the increase in lignoceric acid compared with the wild type control.

The nucleic acid sequence of B2482 from Escherichia coli, e.g. as shown in the respective line in column 5 of Table I, application no. 18, has been published in B. And the activity of the gene product thereof is the activity of NADH dehydrogenase subunit N.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “NADH dehydrogenase subunit N”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said B2482, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said B2482, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said B2482, or a functional equivalent or a homolog thereof as depicted in column 8 of Table II, application no. 18, preferably a homolog or functional equivalent as depicted in column 8 of Table II B, application no. 18, and being depicted in the same respective line as said B2482, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “NADH dehydrogenase subunit N”, preferably it is the molecule of section (a) or (b) of this paragraph.

In particular, it was observed that in plants, especially in Arabidopsis thaliana, increasing or generating the activity of a gene product non-targeted with the activity of a “NADH dehydrogenase subunit N”, preferably being encoded by a gene comprising the nucleic acid sequence SEQ ID NO. 79509, preferably the coding region thereof, conferred the production of or the increase in lignoceric acid compared with the wild type control.

The nucleic acid sequence of B2541 from Escherichia coli, e.g. as shown in the respective line in column 5 of Table I, application no. 18, has been published in B. And the activity of the gene product thereof is the activity of 2,3-dihydroxy-2,3-dihydrophenylpropionatedehydrogenase.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “2,3-dihydroxy-2,3-dihydrophenylpropionatedehydrogenase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said B2541, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said B2541, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said B2541, or a functional equivalent or a homolog thereof as depicted in column 8 of Table II, application no. 18, preferably a homolog or functional equivalent as depicted in column 8 of Table II B, application no. 18, and being depicted in the same respective line as said B2541, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “2,3-dihydroxy-2,3-dihydrophenylpropionatedehydrogenase”, preferably it is the molecule of section (a) or (b) of this paragraph.

In particular, it was observed that in plants, especially in Arabidopsis thaliana, increasing or generating the activity of a gene product non-targeted with the activity of a “2,3-dihydroxy-2,3-dihydrophenylpropionatedehydrogenase”, preferably being encoded by a gene comprising the nucleic acid sequence SEQ ID NO. 40383, preferably the coding region thereof, conferred the production of or the increase in lignoceric acid compared with the wild type control.

The nucleic acid sequence of B3083 from Escherichia coli, e.g. as shown in the respective line in column 5 of Table I, application no. 18, has been published in B. And the activity of the gene product thereof is the activity of b3083-protein.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “b3083-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said B3083, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said B3083, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said B3083, or a functional equivalent or a homolog thereof as depicted in column 8 of Table II, application no. 18, preferably a homolog or functional equivalent as depicted in column 8 of Table II B, application no. 18, and being depicted in the same respective line as said B3083, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “b3083-protein”, preferably it is the molecule of section (a) or (b) of this paragraph.

In particular, it was observed that in plants, especially in Arabidopsis thaliana, increasing or generating the activity of a gene product non-targeted with the activity of a “b3083-protein”, preferably being encoded by a gene comprising the nucleic acid sequence SEQ ID NO. 82424, preferably the coding region thereof, conferred the production of or the increase in lignoceric acid compared with the wild type control.

The nucleic acid sequence of B3812 from Escherichia coli, e.g. as shown in the respective line in column 5 of Table I, application no. 18, has been published in B. And the activity of the gene product thereof is the activity of b3812-protein.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “b3812-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said B3812, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said B3812, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said B3812, or a functional equivalent or a homolog thereof as depicted in column 8 of Table II, application no. 18, preferably a homolog or functional equivalent as depicted in column 8 of Table II B, application no. 18, and being depicted in the same respective line as said B3812, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “b3812-protein”, preferably it is the molecule of section (a) or (b) of this paragraph.

In particular, it was observed that in plants, especially in Arabidopsis thaliana, increasing or generating the activity of a gene product non-targeted with the activity of a “b3812-protein”, preferably being encoded by a gene comprising the nucleic acid sequence SEQ ID NO. 125487, preferably the coding region thereof, conferred the production of or the increase in lignoceric acid compared with the wild type control.

The nucleic acid sequence of B4029 from Escherichia coli, e.g. as shown in the respective line in column 5 of Table I, application no. 18, has been published in B. And the activity of the gene product thereof is the activity of b4029-protein.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “b4029-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said B4029, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said B4029, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said B4029, or a functional equivalent or a homolog thereof as depicted in column 8 of Table II, application no. 18, preferably a homolog or functional equivalent as depicted in column 8 of Table II B, application no. 18, and being depicted in the same respective line as said B4029, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “b4029-protein”, preferably it is the molecule of section (a) or (b) of this paragraph.

In particular, it was observed that in plants, especially in Arabidopsis thaliana, increasing or generating the activity of a gene product non-targeted with the activity of a “b4029-protein”, preferably being encoded by a gene comprising the nucleic acid sequence SEQ ID NO. 10740, preferably the coding region thereof, conferred the production of or the increase in lignoceric acid compared with the wild type control.

The nucleic acid sequence of B4256 from Escherichia coli, e.g. as shown in the respective line in column 5 of Table I, application no. 18, has been published in B. And the activity of the gene product thereof is the activity of acetyltransferase.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “acetyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said B4256, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said B4256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said B4256, or a functional equivalent or a homolog thereof as depicted in column 8 of Table II, application no. 18, preferably a homolog or functional equivalent as depicted in column 8 of Table II B, application no. 18, and being depicted in the same respective line as said B4256, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “acetyltransferase”, preferably it is the molecule of section (a) or (b) of this paragraph.

In particular, it was observed that in plants, especially in Arabidopsis thaliana, increasing or generating the activity of a gene product non-targeted with the activity of a “acetyltransferase”, preferably being encoded by a gene comprising the nucleic acid sequence SEQ ID NO. 45321, preferably the coding region thereof, conferred the production of or the increase in lignoceric acid compared with the wild type control.

The nucleic acid sequence of GM02LC12622 from Glycine max, e.g. as shown in the respective line in column 5 of Table I, application no. 18, is unpublished. And the activity of the gene product thereof is the activity of glutaredoxin.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “glutaredoxin”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said GM02LC12622, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said GM02LC12622, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said GM02LC12622, or a functional equivalent or a homolog thereof as depicted in column 8 of Table II, application no. 18, preferably a homolog or functional equivalent as depicted in column 8 of Table II B, application no. 18, and being depicted in the same respective line as said GM02LC12622, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 10811, preferably the coding region thereof, conferred the production of or the increase in lignoceric acid compared with the wild type control.

The nucleic acid sequence of GM02LC44512 from Glycine max, e.g. as shown in the respective line in column 5 of Table I, application no. 18, is unpublished. And the activity of the gene product thereof is the activity of cyclin D.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “cyclin D”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said GM02LC44512, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said GM02LC44512, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said GM02LC44512, or a functional equivalent or a homolog thereof as depicted in column 8 of Table II, application no. 18, preferably a homolog or functional equivalent as depicted in column 8 of Table II B, application no. 18, and being depicted in the same respective line as said GM02LC44512, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 46850, preferably the coding region thereof, conferred the production of or the increase in lignoceric acid compared with the wild type control.

The nucleic acid sequence of Sll0064 from Synechocystis sp., e.g. as shown in the respective line in column 5 of Table I, application no. 18, has been published in C. And the activity of the gene product thereof is the activity of sll0064-protein.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “sll0064-protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said Sll0064, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said Sll0064, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said Sll0064, or a functional equivalent or a homolog thereof as depicted in column 8 of Table II, application no. 18, preferably a homolog or functional equivalent as depicted in column 8 of Table II B, application no. 18, and being depicted in the same respective line as said Sll0064, and preferably the activity is increased mitochondrial, whereby the respective line disclose in column 7 the fine chemical lignoceric acid.

Accordingly, in one embodiment, the molecule which activity is to be increased in the process of the invention is the gene product with an activity as a “sll0064-protein”, preferably it is the molecule of section (a) or (b) of this paragraph.

In particular, it was observed that in plants, especially in Arabidopsis thaliana, increasing or generating the activity of a gene product mitochondrial with the activity of a “sll0064-protein”, preferably being encoded by a gene comprising the nucleic acid sequence SEQ ID NO. 47526, preferably the coding region thereof, conferred the production of or the increase in lignoceric acid compared with the wild type control.

The nucleic acid sequence of Sll0228 from Synechocystis sp., e.g. as shown in the respective line in column 5 of Table I, application no. 18, has been published in C. And the activity of the gene product thereof is the activity of agmatinase.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “agmatinase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said Sll0228, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said Sll0228, and preferably the activity is increased plastidic, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said Sll0228, or a functional equivalent or a homolog thereof as depicted in column 8 of Table II, application no. 18, preferably a homolog or functional equivalent as depicted in column 8 of Table II B, application no. 18, and being depicted in the same respective line as said Sll0228, and preferably the activity is increased plastidic, whereby the respective line disclose in column 7 the fine chemical lignoceric acid.

Accordingly, in one embodiment, the molecule which activity is to be increased in the process of the invention is the gene product with an activity as a “agmatinase”, preferably it is the molecule of section (a) or (b) of this paragraph.

In particular, it was observed that in plants, especially in Arabidopsis thaliana, increasing or generating the activity of a gene product plastidic with the activity of a “agmatinase”, preferably being encoded by a gene comprising the nucleic acid sequence SEQ ID NO. 84265, preferably the coding region thereof, conferred the production of or the increase in lignoceric acid compared with the wild type control.

The nucleic acid sequence of Sll0378 from Synechocystis sp., e.g. as shown in the respective line in column 5 of Table I, application no. 18, has been published in C. And the activity of the gene product thereof is the activity of uroporphyrin-III C-methyltransferase.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “uroporphyrin-III C-methyltransferase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said Sll0378, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said Sll0378, and preferably the activity is increased plastidic, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said Sll0378, or a functional equivalent or a homolog thereof as depicted in column 8 of Table II, application no. 18, preferably a homolog or functional equivalent as depicted in column 8 of Table II B, application no. 18, and being depicted in the same respective line as said Sll0378, and preferably the activity is increased plastidic, whereby the respective line disclose in column 7 the fine chemical lignoceric acid.

Accordingly, in one embodiment, the molecule which activity is to be increased in the process of the invention is the gene product with an activity as a “uroporphyrin-III C-methyltransferase”, preferably it is the molecule of section (a) or (b) of this paragraph.

In particular, it was observed that in plants, especially in Arabidopsis thaliana, increasing or generating the activity of a gene product plastidic with the activity of a “uroporphyrin-III C-methyltransferase”, preferably being encoded by a gene comprising the nucleic acid sequence SEQ ID NO. 84869, preferably the coding region thereof, conferred the production of or the increase in lignoceric acid compared with the wild type control.

The nucleic acid sequence of Sll0833 from Synechocystis sp., e.g. as shown in the respective line in column 5 of Table I, application no. 18, has been published in C. And the activity of the gene product thereof is the activity of binding-protein-dependent transport systems inner membrane component.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “binding-protein-dependent transport systems inner membrane component”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said Sll0833, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said Sll0833, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said Sll0833, or a functional equivalent or a homolog thereof as depicted in column 8 of Table II, application no. 18, preferably a homolog or functional equivalent as depicted in column 8 of Table II B, application no. 18, and being depicted in the same respective line as said Sll0833, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “binding-protein-dependent transport systems inner membrane component”, preferably it is the molecule of section (a) or (b) of this paragraph.

In particular, it was observed that in plants, especially in Arabidopsis thaliana, increasing or generating the activity of a gene product non-targeted with the activity of a “binding-protein-dependent transport systems inner membrane component”, preferably being encoded by a gene comprising the nucleic acid sequence SEQ ID NO. 101707, preferably the coding region thereof, conferred the production of or the increase in lignoceric acid compared with the wild type control.

The nucleic acid sequence of Sll0891 from Synechocystis sp., e.g. as shown in the respective line in column 5 of Table I, application no. 18, has been published in C. And the activity of the gene product thereof is the activity of malate dehydrogenase.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “malate dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said Sll0891, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said Sll0891, and preferably the activity is increased plastidic, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said Sll0891, or a functional equivalent or a homolog thereof as depicted in column 8 of Table II, application no. 18, preferably a homolog or functional equivalent as depicted in column 8 of Table II B, application no. 18, and being depicted in the same respective line as said Sll0891, and preferably the activity is increased plastidic, whereby the respective line disclose in column 7 the fine chemical lignoceric acid.

Accordingly, in one embodiment, the molecule which activity is to be increased in the process of the invention is the gene product with an activity as a “malate dehydrogenase”, preferably it is the molecule of section (a) or (b) of this paragraph.

In particular, it was observed that in plants, especially in Arabidopsis thaliana, increasing or generating the activity of a gene product plastidic with the activity of a “malate dehydrogenase”, preferably being encoded by a gene comprising the nucleic acid sequence SEQ ID NO. 51268, preferably the coding region thereof, conferred the production of or the increase in lignoceric acid compared with the wild type control.

The nucleic acid sequence of Sll1598 from Synechocystis sp., e.g. as shown in the respective line in column 5 of Table I, application no. 18, has been published in C. And the activity of the gene product thereof is the activity of ABC metal ion transporter substrate-binding protein.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “ABC metal ion transporter substrate-binding protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said Sll1598, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said Sll1598, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said Sll1598, or a functional equivalent or a homolog thereof as depicted in column 8 of Table II, application no. 18, preferably a homolog or functional equivalent as depicted in column 8 of Table II B, application no. 18, and being depicted in the same respective line as said Sll1598, and preferably the activity is increased mitochondrial, whereby the respective line disclose in column 7 the fine chemical lignoceric acid.

Accordingly, in one embodiment, the molecule which activity is to be increased in the process of the invention is the gene product with an activity as a “ABC metal ion transporter substratebinding protein”, preferably it is the molecule of section (a) or (b) of this paragraph.

In particular, it was observed that in plants, especially in Arabidopsis thaliana, increasing or generating the activity of a gene product mitochondrial with the activity of a “ABC metal ion transporter substrate-binding protein”, preferably being encoded by a gene comprising the nucleic acid sequence SEQ ID NO. 85743, preferably the coding region thereof, conferred the production of or the increase in lignoceric acid compared with the wild type control.

The nucleic acid sequence of Sll1848 from Synechocystis sp., e.g. as shown in the respective line in column 5 of Table I, application no. 18, has been published in C. And the activity of the gene product thereof is the activity of acyl transferase.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “acyl transferase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said Sll1848, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said Sll1848, and preferably the activity is increased plastidic, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said Sll1848, or a functional equivalent or a homolog thereof as depicted in column 8 of Table II, application no. 18, preferably a homolog or functional equivalent as depicted in column 8 of Table II B, application no. 18, and being depicted in the same respective line as said Sll1848, and preferably the activity is increased plastidic, whereby the respective line disclose in column 7 the fine chemical lignoceric acid.

Accordingly, in one embodiment, the molecule which activity is to be increased in the process of the invention is the gene product with an activity as a “acyl transferase”, preferably it is the molecule of section (a) or (b) of this paragraph.

In particular, it was observed that in plants, especially in Arabidopsis thaliana, increasing or generating the activity of a gene product plastidic with the activity of a “acyl transferase”, preferably being encoded by a gene comprising the nucleic acid sequence SEQ ID NO. 86447, preferably the coding region thereof, conferred the production of or the increase in lignoceric acid compared with the wild type control.

The nucleic acid sequence of Slr0383 from Synechocystis sp., e.g. as shown in the respective line in column 5 of Table I, application no. 18, has been published in C. And the activity of the gene product thereof is the activity of slr0383-protein.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “slr0383-protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said Slr0383, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said Slr0383, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said Slr0383, or a functional equivalent or a homolog thereof as depicted in column 8 of Table II, application no. 18, preferably a homolog or functional equivalent as depicted in column 8 of Table II B, application no. 18, and being depicted in the same respective line as said Slr0383, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “slr0383-protein”, preferably it is the molecule of section (a) or (b) of this paragraph.

In particular, it was observed that in plants, especially in Arabidopsis thaliana, increasing or generating the activity of a gene product non-targeted with the activity of a “slr0383-protein”, preferably being encoded by a gene comprising the nucleic acid sequence SEQ ID NO. 95744, preferably the coding region thereof, conferred the production of or the increase in lignoceric acid compared with the wild type control.

The nucleic acid sequence of Slr0739 from Synechocystis sp., e.g. as shown in the respective line in column 5 of Table I, application no. 18, has been published in C. And the activity of the gene product thereof is the activity of geranylgeranyl pyrophosphate synthase.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “geranylgeranyl pyrophosphate synthase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said Slr0739, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said Slr0739, and preferably the activity is increased plastidic, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said Slr0739, or a functional equivalent or a homolog thereof as depicted in column 8 of Table II, application no. 18, preferably a homolog or functional equivalent as depicted in column 8 of Table II B, application no. 18, and being depicted in the same respective line as said Slr0739, and preferably the activity is increased plastidic, whereby the respective line disclose in column 7 the fine chemical lignoceric acid.

Accordingly, in one embodiment, the molecule which activity is to be increased in the process of the invention is the gene product with an activity as a “geranylgeranyl pyrophosphate synthase”, preferably it is the molecule of section (a) or (b) of this paragraph.

In particular, it was observed that in plants, especially in Arabidopsis thaliana, increasing or generating the activity of a gene product plastidic with the activity of a “geranylgeranyl pyrophosphate synthase”, preferably being encoded by a gene comprising the nucleic acid sequence SEQ ID NO. 57235, preferably the coding region thereof, conferred the production of or the increase in lignoceric acid compared with the wild type control.

The nucleic acid sequence of Slr0966 from Synechocystis sp., e.g. as shown in the respective line in column 5 of Table I, application no. 18, has been published in C. And the activity of the gene product thereof is the activity of tryptophan synthase alpha chain.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “tryptophan synthase alpha chain”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said Slr0966, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said Slr0966, and preferably the activity is increased plastidic, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said Slr0966, or a functional equivalent or a homolog thereof as depicted in column 8 of Table II, application no. 18, preferably a homolog or functional equivalent as depicted in column 8 of Table II B, application no. 18, and being depicted in the same respective line as said Slr0966, and preferably the activity is increased plastidic, whereby the respective line disclose in column 7 the fine chemical lignoceric acid.

Accordingly, in one embodiment, the molecule which activity is to be increased in the process of the invention is the gene product with an activity as a “tryptophan synthase alpha chain”, preferably it is the molecule of section (a) or (b) of this paragraph.

In particular, it was observed that in plants, especially in Arabidopsis thaliana, increasing or generating the activity of a gene product plastidic with the activity of a “tryptophan synthase alpha chain”, preferably being encoded by a gene comprising the nucleic acid sequence SEQ ID NO. 87655, preferably the coding region thereof, conferred the production of or the increase in lignoceric acid compared with the wild type control.

The nucleic acid sequence of Slr2033 from Synechocystis sp., e.g. as shown in the respective line in column 5 of Table I, application no. 18, has been published in C. And the activity of the gene product thereof is the activity of rubredoxin.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “rubredoxin”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said Slr2033, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said Slr2033, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said Slr2033, or a functional equivalent or a homolog thereof as depicted in column 8 of Table II, application no. 18, preferably a homolog or functional equivalent as depicted in column 8 of Table II B, application no. 18, and being depicted in the same respective line as said Slr2033, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “rubredoxin”, preferably it is the molecule of section (a) or (b) of this paragraph.

In particular, it was observed that in plants, especially in Arabidopsis thaliana, increasing or generating the activity of a gene product non-targeted with the activity of a “rubredoxin”, preferably being encoded by a gene comprising the nucleic acid sequence SEQ ID NO. 152275, preferably the coding region thereof, conferred the production of or the increase in lignoceric acid compared with the wild type control.

The nucleic acid sequence of TTC0019 from Thermus thermophilus, e.g. as shown in the respective line in column 5 of Table I, application no. 18, has been published in E. And the activity of the gene product thereof is the activity of Sec-independent protein translocase subunit.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “Sec-independent protein translocase subunit”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said TTC0019, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said TTC0019, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said TTC0019, or a functional equivalent or a homolog thereof as depicted in column 8 of Table II, application no. 18, preferably a homolog or functional equivalent as depicted in column 8 of Table II B, application no. 18, and being depicted in the same respective line as said TTC0019, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “Sec-independent protein translocase subunit”, preferably it is the molecule of section (a) or (b) of this paragraph.

In particular, it was observed that in plants, especially in Arabidopsis thaliana, increasing or generating the activity of a gene product non-targeted with the activity of a “Sec-independent protein translocase subunit”, preferably being encoded by a gene comprising the nucleic acid sequence SEQ ID NO. 12698, preferably the coding region thereof, conferred the production of or the increase in lignoceric acid compared with the wild type control.

The nucleic acid sequence of TTC0768 from Thermus thermophilus, e.g. as shown in the respective line in column 5 of Table I, application no. 18, has been published in E. And the activity of the gene product thereof is the activity of TTC0768-protein.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “TTC0768-protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said TTC0768, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said TTC0768, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said TTC0768, or a functional equivalent or a homolog thereof as depicted in column 8 of Table II, application no. 18, preferably a homolog or functional equivalent as depicted in column 8 of Table II B, application no. 18, and being depicted in the same respective line as said TTC0768, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “TTC0768-protein”, preferably it is the molecule of section (a) or (b) of this paragraph.

In particular, it was observed that in plants, especially in Arabidopsis thaliana, increasing or generating the activity of a gene product non-targeted with the activity of a “TTC0768-protein”, preferably being encoded by a gene comprising the nucleic acid sequence SEQ ID NO. 61532, preferably the coding region thereof, conferred the production of or the increase in lignoceric acid compared with the wild type control.

The nucleic acid sequence of TTC1386 from Thermus thermophilus, e.g. as shown in the respective line in column 5 of Table I, application no. 18, has been published in E. And the activity of the gene product thereof is the activity of TTC1386-protein.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “TTC1386-protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said TTC1386, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said TTC1386, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said TTC1386, or a functional equivalent or a homolog thereof as depicted in column 8 of Table II, application no. 18, preferably a homolog or functional equivalent as depicted in column 8 of Table II B, application no. 18, and being depicted in the same respective line as said TTC1386, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “TTC1386-protein”, preferably it is the molecule of section (a) or (b) of this paragraph.

In particular, it was observed that in plants, especially in Arabidopsis thaliana, increasing or generating the activity of a gene product non-targeted with the activity of a “TTC1386-protein”, preferably being encoded by a gene comprising the nucleic acid sequence SEQ ID NO. 62079, preferably the coding region thereof, conferred the production of or the increase in lignoceric acid compared with the wild type control.

The nucleic acid sequence of Yal054c from Saccharomyces cerevisiae, e.g. as shown in the respective line in column 5 of Table I, application no. 18, has been published in A. And the activity of the gene product thereof is the activity of acetyl-coenzyme A synthetase.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “acetyl-coenzyme A synthetase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said Yal054c, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said Yal054c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said Yal054c, or a functional equivalent or a homolog thereof as depicted in column 8 of Table II, application no. 18, preferably a homolog or functional equivalent as depicted in column 8 of Table II B, application no. 18, and being depicted in the same respective line as said Yal054c, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “acetyl-coenzyme A synthetase”, preferably it is the molecule of section (a) or (b) of this paragraph.

In particular, it was observed that in plants, especially in Arabidopsis thaliana, increasing or generating the activity of a gene product non-targeted with the activity of a “acetyl-coenzyme A synthetase”, preferably being encoded by a gene comprising the nucleic acid sequence SEQ ID NO. 121908, preferably the coding region thereof, conferred the production of or the increase in lignoceric acid compared with the wild type control.

The nucleic acid sequence of Ydr265w from Saccharomyces cerevisiae, e.g. as shown in the respective line in column 5 of Table I, application no. 18, has been published in A. And the activity of the gene product thereof is the activity of peroxisome assembly protein.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “peroxisome assembly protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said Ydr265w, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said Ydr265w, and preferably the activity is increased plastidic, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said Ydr265w, or a functional equivalent or a homolog thereof as depicted in column 8 of Table II, application no. 18, preferably a homolog or functional equivalent as depicted in column 8 of Table II B, application no. 18, and being depicted in the same respective line as said Ydr265w, and preferably the activity is increased plastidic, whereby the respective line disclose in column 7 the fine chemical lignoceric acid.

Accordingly, in one embodiment, the molecule which activity is to be increased in the process of the invention is the gene product with an activity as a “peroxisome assembly protein”, preferably it is the molecule of section (a) or (b) of this paragraph.

In particular, it was observed that in plants, especially in Arabidopsis thaliana, increasing or generating the activity of a gene product plastidic with the activity of a “peroxisome assembly protein”, preferably being encoded by a gene comprising the nucleic acid sequence SEQ ID NO. 63665, preferably the coding region thereof, conferred the production of or the increase in lignoceric acid compared with the wild type control.

The nucleic acid sequence of Yjl153c from Saccharomyces cerevisiae, e.g. as shown in the respective line in column 5 of Table I, application no. 18, has been published in A. And the activity of the gene product thereof is the activity of inositol-3-phosphate synthase.

Accordingly, in one embodiment, the process of the present invention for producing heneicosanoic acid in a non-human organism, like a microorganism or a plant or a part thereof, comprises increasing or generating the activity of a gene product with the activity of a gene product conferring the activity of “inositol-3-phosphate synthase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical heneicosanoic acid), application no. 18, preferably the coding region thereof, or a homolog or a fragment thereof, and being depicted in the same respective line as said Yjl153c, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said Yjl153c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said Yjl153c, or a functional equivalent or a homolog thereof as depicted in column 8 of Table II, application no. 18, preferably a homolog or functional equivalent as depicted in column 8 of Table II B, application no. 18, and being depicted in the same respective line as said Yjl153c, and preferably the activity is increased non-targeted, whereby the respective line disclose in column 7 the fine chemical heneicosanoic acid.

Accordingly, in one embodiment, the molecule which activity is to be increased in the process of the invention is the gene product with an activity as a “inositol-3-phosphate synthase”, preferably it is the molecule of section (a) or (b) of this paragraph.

In particular, it was observed that in plants, especially in Arabidopsis thaliana, increasing or generating the activity of a gene product non-targeted with the activity of a “inositol-3-phosphate synthase”, preferably being encoded by a gene comprising the nucleic acid sequence SEQ ID NO. 149632, preferably the coding region thereof, conferred the production of or the increase in heneicosanoic acid compared with the wild type control.

The nucleic acid sequence of Yjr130c from Saccharomyces cerevisiae, e.g. as shown in the respective line in column 5 of Table I, application no. 18, has been published in A. And the activity of the gene product thereof is the activity of cystathionine gamma-synthase.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “cystathionine gamma-synthase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said Yjr130c, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said Yjr130c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said Yjr130c, or a functional equivalent or a homolog thereof as depicted in column 8 of Table II, application no. 18, preferably a homolog or functional equivalent as depicted in column 8 of Table II B, application no. 18, and being depicted in the same respective line as said Yjr130c, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “cystathionine gamma-synthase”, preferably it is the molecule of section (a) or (b) of this paragraph.

In particular, it was observed that in plants, especially in Arabidopsis thaliana, increasing or generating the activity of a gene product non-targeted with the activity of a “cystathionine gamma-synthase”, preferably being encoded by a gene comprising the nucleic acid sequence SEQ ID NO. 14843, preferably the coding region thereof, conferred the production of or the increase in lignoceric acid compared with the wild type control.

The nucleic acid sequence of Ykr050w from Saccharomyces cerevisiae, e.g. as shown in the respective line in column 5 of Table I, application no. 18, has been published in A. And the activity of the gene product thereof is the activity of potassium transport protein.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “potassium transport protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said Ykr050w, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said Ykr050w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said Ykr050w, or a functional equivalent or a homolog thereof as depicted in column 8 of Table II, application no. 18, preferably a homolog or functional equivalent as depicted in column 8 of Table II B, application no. 18, and being depicted in the same respective line as said Ykr050w, and preferably the activity is increased non-targeted, whereby the respective line disclose in column 7 the fine chemical lignoceric acid.

Accordingly, in one embodiment, the molecule which activity is to be increased in the process of the invention is the gene product with an activity as a “potassium transport protein”, preferably it is the molecule of section (a) or (b) of this paragraph.

In particular, it was observed that in plants, especially in Arabidopsis thaliana, increasing or generating the activity of a gene product non-targeted with the activity of a “potassium transport protein”, preferably being encoded by a gene comprising the nucleic acid sequence SEQ ID NO. 152382, preferably the coding region thereof, conferred the production of or the increase in lignoceric acid compared with the wild type control.

The nucleic acid sequence of Ylr065c from Saccharomyces cerevisiae, e.g. as shown in the respective line in column 5 of Table I, application no. 18, has been published in A. And the activity of the gene product thereof is the activity of ylr065c-protein.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “ylr065c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said Ylr065c, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said Ylr065c, and preferably the activity is increased plastidic, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said Ylr065c, or a functional equivalent or a homolog thereof as depicted in column 8 of Table II, application no. 18, preferably a homolog or functional equivalent as depicted in column 8 of Table II B, application no. 18, and being depicted in the same respective line as said Ylr065c, and preferably the activity is increased plastidic, whereby the respective line disclose in column 7 the fine chemical lignoceric acid.

Accordingly, in one embodiment, the molecule which activity is to be increased in the process of the invention is the gene product with an activity as a “ylr065c-protein”, preferably it is the molecule of section (a) or (b) of this paragraph.

In particular, it was observed that in plants, especially in Arabidopsis thaliana, increasing or generating the activity of a gene product plastidic with the activity of a “ylr065c-protein”, preferably being encoded by a gene comprising the nucleic acid sequence SEQ ID NO. 66695, preferably the coding region thereof, conferred the production of or the increase in lignoceric acid compared with the wild type control.

The nucleic acid sequence of Yml093w from Saccharomyces cerevisiae, e.g. as shown in the respective line in column 5 of Table I, application no. 18, has been published in A. And the activity of the gene product thereof is the activity of yml093w-protein.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “yml093w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said Yml093w, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said Yml093w, and preferably the activity is increased plastidic, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said Yml093w, or a functional equivalent or a homolog thereof as depicted in column 8 of Table II, application no. 18, preferably a homolog or functional equivalent as depicted in column 8 of Table II B, application no. 18, and being depicted in the same respective line as said Yml093w, and preferably the activity is increased plastidic, whereby the respective line disclose in column 7 the fine chemical lignoceric acid.

Accordingly, in one embodiment, the molecule which activity is to be increased in the process of the invention is the gene product with an activity as a “yml093w-protein”, preferably it is the molecule of section (a) or (b) of this paragraph.

In particular, it was observed that in plants, especially in Arabidopsis thaliana, increasing or generating the activity of a gene product plastidic with the activity of a “yml093w-protein”, preferably being encoded by a gene comprising the nucleic acid sequence SEQ ID NO. 152424, preferably the coding region thereof, conferred the production of or the increase in lignoceric acid compared with the wild type control.

The nucleic acid sequence of Ymr150c from Saccharomyces cerevisiae, e.g. as shown in the respective line in column 5 of Table I, application no. 18, has been published in A. And the activity of the gene product thereof is the activity of mitochondrial inner membrane protease subunit.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “mitochondrial inner membrane protease subunit”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said Ymr150c, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said Ymr150c, and preferably the activity is increased plastidic, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said Ymr150c, or a functional equivalent or a homolog thereof as depicted in column 8 of Table II, application no. 18, preferably a homolog or functional equivalent as depicted in column 8 of Table II B, application no. 18, and being depicted in the same respective line as said Ymr150c, and preferably the activity is increased plastidic, whereby the respective line disclose in column 7 the fine chemical lignoceric acid.

Accordingly, in one embodiment, the molecule which activity is to be increased in the process of the invention is the gene product with an activity as a “mitochondrial inner membrane protease subunit”, preferably it is the molecule of section (a) or (b) of this paragraph.

In particular, it was observed that in plants, especially in Arabidopsis thaliana, increasing or generating the activity of a gene product plastidic with the activity of a “mitochondrial inner membrane protease subunit”, preferably being encoded by a gene comprising the nucleic acid sequence SEQ ID NO. 92274, preferably the coding region thereof, conferred the production of or the increase in lignoceric acid compared with the wild type control.

The nucleic acid sequence of Ymr208w from Saccharomyces cerevisiae, e.g. as shown in the respective line in column 5 of Table I, application no. 18, has been published in A. And the activity of the gene product thereof is the activity of mevalonate kinase.

Accordingly, in one embodiment, the process of the present invention for producing lignoceric acid in a non-human organism, like a microorganism or a plant or a part thereof, comprises increasing or generating the activity of a gene product with the activity of a gene product conferring the activity of “mevalonate kinase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical lignoceric acid), application no. 18, preferably the coding region thereof, or a homolog or a fragment thereof, and being depicted in the same respective line as said Ymr208w, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said Ymr208w, and preferably the activity is increased plastidic, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said Ymr208w, or a functional equivalent or a homolog thereof as depicted in column 8 of Table II, application no. 18, preferably a homolog or functional equivalent as depicted in column 8 of Table II B, application no. 18, and being depicted in the same respective line as said Ymr208w, and preferably the activity is increased plastidic, whereby the respective line disclose in column 7 the fine chemical lignoceric acid.

Accordingly, in one embodiment, the molecule which activity is to be increased in the process of the invention is the gene product with an activity as a “mevalonate kinase”, preferably it is the molecule of section (a) or (b) of this paragraph.

In particular, it was observed that in plants, especially in Arabidopsis thaliana, increasing or generating the activity of a gene product plastidic with the activity of a “mevalonate kinase”, preferably being encoded by a gene comprising the nucleic acid sequence SEQ ID NO. 152460, preferably the coding region thereof, conferred the production of or the increase in lignoceric acid compared with the wild type control.

The nucleic acid sequence of Yer152c from Saccharomyces cerevisiae, e.g. as shown in the respective line in column 5 of Table I, application no. 18, has been published in A. And the activity of the gene product thereof is the activity of yer152c-protein.

Accordingly, in one embodiment, the process of the present invention for producing melissic acid in a non-human organism, like a microorganism or a plant or a part thereof, comprises increasing or generating the activity of a gene product with the activity of a gene product conferring the activity of “yer152c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical melissic acid), application no. 18, preferably the coding region thereof, or a homolog or a fragment thereof, and being depicted in the same respective line as said Yer152c, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said Yer152c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said Yer152c, or a functional equivalent or a homolog thereof as depicted in column 8 of Table II, application no. 18, preferably a homolog or functional equivalent as depicted in column 8 of Table II B, application no. 18, and being depicted in the same respective line as said Yer152c, and preferably the activity is increased non-targeted, whereby the respective line disclose in column 7 the fine chemical melissic acid.

Accordingly, in one embodiment, the molecule which activity is to be increased in the process of the invention is the gene product with an activity as a “yer152c-protein”, preferably it is the molecule of section (a) or (b) of this paragraph.

In particular, it was observed that in plants, especially in Arabidopsis thaliana, increasing or generating the activity of a gene product non-targeted with the activity of a “yer152c-protein”, preferably being encoded by a gene comprising the nucleic acid sequence SEQ ID NO. 152341, preferably the coding region thereof, conferred the production of or the increase in melissic acid compared with the wild type control.

The nucleic acid sequence of Yer152c from Saccharomyces cerevisiae, e.g. as shown in the respective line in column 5 of Table I, application no. 18, has been published in A. And the activity of the gene product thereof is the activity of yer152c-protein.

Accordingly, in one embodiment, the process of the present invention for producing montanic acid in a non-human organism, like a microorganism or a plant or a part thereof, comprises increasing or generating the activity of a gene product with the activity of a gene product conferring the activity of “yer152c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical montanic acid), application no. 18, preferably the coding region thereof, or a homolog or a fragment thereof, and being depicted in the same respective line as said Yer152c, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said Yer152c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said Yer152c, or a functional equivalent or a homolog thereof as depicted in column 8 of Table II, application no. 18, preferably a homolog or functional equivalent as depicted in column 8 of Table II B, application no. 18, and being depicted in the same respective line as said Yer152c, and preferably the activity is increased non-targeted, whereby the respective line disclose in column 7 the fine chemical montanic acid.

Accordingly, in one embodiment, the molecule which activity is to be increased in the process of the invention is the gene product with an activity as a “yer152c-protein”, preferably it is the molecule of section (a) or (b) of this paragraph.

In particular, it was observed that in plants, especially in Arabidopsis thaliana, increasing or generating the activity of a gene product non-targeted with the activity of a “yer152c-protein”, preferably being encoded by a gene comprising the nucleic acid sequence SEQ ID NO. 152341, preferably the coding region thereof, conferred the production of or the increase in montanic acid compared with the wild type control.

The nucleic acid sequence of B3644 from Escherichia coli, e.g. as shown in the respective line in column 5 of Table I, application no. 18, has been published in B. And the activity of the gene product thereof is the activity of b3644-protein.

Accordingly, in one embodiment, the process of the present invention for producing melissic acid in a non-human organism, like a microorganism or a plant or a part thereof, comprises increasing or generating the activity of a gene product with the activity of a gene product conferring the activity of “b3644-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical melissic acid), application no. 18, preferably the coding region thereof, or a homolog or a fragment thereof, and being depicted in the same respective line as said B3644, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said B3644, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said B3644, or a functional equivalent or a homolog thereof as depicted in column 8 of Table II, application no. 18, preferably a homolog or functional equivalent as depicted in column 8 of Table II B, application no. 18, and being depicted in the same respective line as said B3644, and preferably the activity is increased non-targeted, whereby the respective line disclose in column 7 the fine chemical melissic acid.

Accordingly, in one embodiment, the molecule which activity is to be increased in the process of the invention is the gene product with an activity as a “b3644-protein”, preferably it is the molecule of section (a) or (b) of this paragraph.

In particular, it was observed that in plants, especially in Arabidopsis thaliana, increasing or generating the activity of a gene product non-targeted with the activity of a “b3644-protein”, preferably being encoded by a gene comprising the nucleic acid sequence SEQ ID NO. 125348, preferably the coding region thereof, conferred the production of or the increase in melissic acid compared with the wild type control.

The nucleic acid sequence of B3644 from Escherichia coli, e.g. as shown in the respective line in column 5 of Table I, application no. 18, has been published in B. And the activity of the gene product thereof is the activity of b3644-protein.

Accordingly, in one embodiment, the process of the present invention for producing montanic acid in a non-human organism, like a microorganism or a plant or a part thereof, comprises increasing or generating the activity of a gene product with the activity of a gene product conferring the activity of “b3644-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical montanic acid), application no. 18, preferably the coding region thereof, or a homolog or a fragment thereof, and being depicted in the same respective line as said B3644, or a functional equivalent or a homolog thereof as shown in column 8 of Table I, application no. 18, preferably the coding region thereof, particularly a homolog or functional equivalent as shown in column 8 of Table I B, application no. 18, and being depicted in the same respective line as said B3644, and preferably the activity is increased non-targeted, or
(b) a polypeptide comprising a polypeptide, a consensus sequence or at least a polypeptide motif as shown in the respective line in column 5 of Table II or in column 8 of Table IV, application no. 18, respectively, and being depicted in the same respective line as said B3644, or a functional equivalent or a homolog thereof as depicted in column 8 of Table II, application no. 18, preferably a homolog or functional equivalent as depicted in column 8 of Table II B, application no. 18, and being depicted in the same respective line as said B3644, and preferably the activity is increased non-targeted, whereby the respective line disclose in column 7 the fine chemical montanic acid.

Accordingly, in one embodiment, the molecule which activity is to be increased in the process of the invention is the gene product with an activity as a “b3644-protein”, preferably it is the molecule of section (a) or (b) of this paragraph.

In particular, it was observed that in plants, especially in Arabidopsis thaliana, increasing or generating the activity of a gene product non-targeted with the activity of a “b3644-protein”, preferably being encoded by a gene comprising the nucleic acid sequence SEQ ID NO. 125348, preferably the coding region thereof, conferred the production of or the increase in montanic acid compared with the wild type control.

[0105.1.7.18] to [0107.1.7.18] for the disclosure of these paragraphs see [0105.1.7.7] to [0107.1.7.7] above

A protein having an activity conferring an increase in the amount or level of the fine chemical cerotic acid, heneicosanoic acid, lignoceric acid, melissic acid or montanic acid, upon targeting to the plastids or mitochondria or upon non-targeting, preferably has the structure of the respective polypeptide described herein, in particular of the polypeptides comprising the consensus sequence or at least one polypeptide motifs as shown in the respective line in Table IV, application no. 18, column 8 or of the polypeptide comprising an amino acid sequence as disclosed in the respective line in Table II, application no. 18, columns 5 or 8, or homologs or fragments thereof as described herein, or is encoded by the nucleic acid molecule characterized herein or the nucleic acid molecule according to the invention, for example by the nucleic acid molecule as shown in the respective line in Table I, application no. 18, columns 5 or 8, preferably the coding region thereof, or homologs or fragments thereof and has the herein mentioned activity.

for the disclosure of this paragraph see [0109.1.7.7] above

for the disclosure of this paragraph see [0110.1.7.7] above

In one embodiment, the process of the present invention comprises one or more of the following steps
(a) stabilizing a protein conferring the generation or increased expression of a protein encoded by the nucleic acid molecule of the invention or of the polypeptide of the invention, e.g. of a polypeptide having the activity selected from the group consisting of 2,3-dihydroxy-2,3-dihydrophenylpropionatedehydrogenase, ABC metal ion transporter substrate-binding protein, acetyl-coenzyme A synthetase, acetyltransferase, acid shock protein, acyl transferase, agmatinase, aminotransferase, ATP-binding component of a transport system, b1522-protein, b3083-protein, b3644-protein, b3812-protein, b4029-protein, binding-protein-dependent transport systems inner membrane component, calcium-dependent protein kinase, cardiolipin synthase, chaperone protein CIpB, cyclin D, cystathionine gamma-synthase, eukaryotic translation initiation factor 5, geranylgeranyl pyrophosphate synthase, gibberellin 20-oxidase, glutamate-ammonia-ligase, glutaredoxin, glycosyl transferase, heat shock transcription factor, hydrolase, inositol-3-phosphate synthase, isochorismate synthase, leucyl/phenylalanyl-tRNA-protein transferase, major facilitator superfamily transporter protein, malate dehydrogenase, mevalonate kinase, mitochondrial inner membrane protease subunit, monodehydroascorbate reductase, multidrug resistance protein, NADH dehydrogenase subunit N, O-antigen chain length determinant, oxidoreductase, peroxisome assembly protein, potassium transport protein, protein kinase, purine nucleoside phosphorylase, rubredoxin, Sec-independent protein translocase subunit, serine protease, sll0064-protein, slr0383-protein, sodium/proton antiporter, thioredoxin family protein, threonine synthase, tryptophan synthase alpha chain, TTC0768-protein, TTC1386-protein, uroporphyrin-III C-methyltransferase, yer152c-protein, ylr065c-protein, yml093w-protein, and zinc finger protein, or of a polypeptide as indicated in the respective line in Table II, application no. 18, columns 5 or 8, or its homologs or fragments, and conferring the production of or an increase in cerotic acid, heneicosanoic acid, lignoceric acid, melissic acid or montanic acid, respectively; and/or
(b) stabilizing a mRNA conferring the generation or increased expression of a FCRP, e.g. protein encoded by the nucleic acid molecule of the invention or its homologs or fragments, or of a mRNA encoding the polypeptide of the present invention having the hereinmentioned activity selected from the group consisting of said activities mentioned in (a) and conferring the production of or an increase in cerotic acid, heneicosanoic acid, lignoceric acid, melissic acid or montanic acid, respectively; and/or
(c) increasing the specific activity of a protein conferring the increased expression of a FCRP, e.g. a protein encoded by the nucleic acid molecule of the invention or of the polypeptide of the present invention having herein-mentioned cerotic acid, heneicosanoic acid, lignoceric acid, melissic acid or montanic acid generating or increasing activity, respectively, e.g. of a polypeptide having the activity of a protein as indicated in the respective line in Table II, application no. 18, columns 5 or 8, or its homologs or fragments, or decreasing the inhibitory regulation of the polypeptide of the invention; and/or
(d) generating or increasing the expression of an endogenous or artificial transcription factor mediating the expression of a protein conferring the generation or increased expression of a FCRP, e.g. a protein encoded by the nucleic acid molecule of the invention or of the polypeptide of the invention having herein-mentioned activity selected from the group consisting of said activities mentioned in (a) and conferring a cerotic acid, heneicosanoic acid, lignoceric acid, melissic acid or montanic acid increasing activity, respectively, e.g. of a polypeptide having the activity of a protein as indicated in the respective line in Table II, application no. 18, columns 5 or 8 or its homologs; and/or
(e) stimulating activity of a protein conferring the increased expression of a FCRP, e.g. a protein encoded by the nucleic acid molecule of the present invention or a polypeptide of the present invention having herein-mentioned activity selected from the group consisting of said activities mentioned in (a) and conferring a cerotic acid, heneicosanoic acid, lignoceric acid, melissic acid or montanic acid increasing activity, respectively, e.g. of a polypeptide having the activity of a protein as indicated in the respective line in Table II, application no. 18, columns 5 or 8, or its homologs or fragments, by adding one or more exogenous inducing factors to the non-human organism or parts thereof; and/or
(f) expressing a transgenic gene encoding a protein conferring the increased expression of a FCRP, e.g. a polypeptide encoded by the nucleic acid molecule of the present invention or a polypeptide of the present invention, having herein-mentioned activity selected from the group consisting of said activities mentioned in (a) and conferring a cerotic acid, heneicosanoic acid, lignoceric acid, melissic acid or montanic acid increasing activity, respectively, e.g. of a polypeptide having the activity of a protein as indicated in the respective line in Table II, application no. 18, columns 5 or 8, or its homologs or fragments, and/or
(g) increasing the copy number of a gene conferring the increased expression of a nucleic acid molecule encoding a FCRP, e.g. a polypeptide encoded by the nucleic acid molecule of the invention or the polypeptide of the invention having herein-mentioned activity selected from the group consisting of said activities mentioned in (a) and conferring a cerotic acid, heneicosanoic acid, lignoceric acid, melissic acid or montanic acid increasing activity, respectively, e.g. of a polypeptide having the activity of a protein as indicated in the respective line in Table II, application no. 18, columns 5 or 8, or its homologs or fragments; and/or
(h) increasing the expression of the endogenous gene encoding the FCRP, polypeptide of the invention, e.g. a polypeptide encoded by the nucleic acid molecule of the invention or the polypeptide of the invention having herein-mentioned activity selected from the group consisting of said activities mentioned in (a) and conferring a cerotic acid, heneicosanoic acid, lignoceric acid, melissic acid or montanic acid; increasing activity, respectively, e.g. of a polypeptide having the activity of a protein as indicated in the respective line in Table II, application no. 18, columns 5 or 8, or its homologs or fragments, by adding positive expression or removing negative expression elements, e.g. homologous recombination can be used to either introduce positive regulatory elements like for plants the 35S enhancer into the promoter or to remove repressor elements form regulatory regions. Further gene conversion methods can be used to disrupt repressor elements or to enhance to activity of positive elements. Positive elements can be randomly introduced in plants by T-DNA or transposon mutagenesis and lines can be identified in which the positive elements have be integrated near to a gene of the invention, the expression of which is thereby enhanced; and/or
(i) modulating growth conditions of the non-human organism in such a manner, that the expression or activity of the gene encoding the FCRP, e.g. a protein of the invention or the protein itself is enhanced, for example microorganisms or plants can be grown for example under a higher temperature regime leading to an enhanced expression of heat shock proteins, which can lead to an enhanced cerotic acid, heneicosanoic acid, lignoceric acid, melissic acid or montanic acid production, respectively; and/or
(j) selecting of non-human organisms with especially high activity of the proteins of the invention from natural or from mutagenized resources and breeding them into the target organisms, e.g. the elite crops; and/or
(k) directing a protein encoded by the nucleic acid molecule of the invention or of the polypeptide of the present invention having herein-mentioned activity selected from the group consisting of said activities mentioned in (a) and conferring a cerotic acid, heneicosanoic acid, lignoceric acid, melissic acid or montanic acid increasing activity, respectively, e.g. of a polypeptide having the activity of a protein as indicated in the respective line in Table II, application no. 18, columns 5 or 8, or its homologs or fragments, if for the corresponding nucleic acid molecule in the respective line in column 6 of Table I the term “plastidic” is indicated, to the plastids by the addition of a plastidial targeting sequence or if for the corresponding nucleic acid molecule in the respective line in column 6 of Table I the term “mitochondric” is indicated, to the mitochondria by the addition of a mitochondrial targeting sequence; and/or
(l) generating the expression of a protein encoded by the nucleic acid molecule of the invention or of the polypeptide of the present invention having herein-mentioned activity selected from the group consisting of said activities mentioned in (a) and conferring a cerotic acid, heneicosanoic acid, lignoceric acid, melissic acid or montanic acid increasing activity, respectively, e.g. of a polypeptide having the activity of a protein as indicated in the respective line in Table II, application no. 18, columns 5 or 8, or its homologs or fragments, if for the corresponding nucleic acid molecule in the respective line in column 6 of Table I the term “plastidic” or “mitochondric” is indicated, in these organelles by the stable or transient transformation, advantageously stable transformation, of organelles, preferably plastids or mitochondria, with an inventive nucleic acid sequence preferably in form of an expression cassette containing said sequence leading to the expression of the nucleic acids or polypeptides of the invention in the respective organelle; and/or
(m) generating the expression of a protein encoded by the nucleic acid molecule of the invention or of the polypeptide of the present invention having herein-mentioned activity selected from the group consisting of said activities mentioned in (a) and conferring a cerotic acid, heneicosanoic acid, lignoceric acid, melissic acid and/or montanic acid increasing activity, respectively, e.g. of a polypeptide having the activity of a protein as indicated in the respective line in Table II, application no. 18, columns 5 or 8, or its homologs or fragments, if for the corresponding nucleic acid molecule in the respective line in column 6 of Table I the term “plastidic” or “mitochondric” is indicated, in these organelles by integration of a nucleic acid of the invention into the genome of the respective organelle under control of preferable a promoter selective for the respective organelle.

Preferably, said mRNA is the coding region of the nucleic acid molecule of the present invention and/or the protein conferring the increased expression of a protein encoded by the coding region of the nucleic acid molecule of the present invention alone or linked to a transit nucleic acid sequence or transit peptide encoding nucleic acid sequence or the polypeptide having the herein mentioned activity, e.g. conferring the generation of or increase of cerotic acid, heneicosanoic acid, lignoceric acid, melissic acid or montanic acid, respectively, after increasing the expression or activity of the encoded polypeptide, non-targeted or in organelles such as plastids and/or mitochondria, preferably plastids, or having the activity of a polypeptide having an activity as the protein as shown in the respective line in Table II, application no. 18, column 3, or its homologs. Preferably the increase of cerotic acid, heneicosanoic acid, lignoceric acid, melissic acid or montanic acid, respectively, takes place non-targeted or in plastids and/or mitochondria, preferably non-targeted or in plastids.

[0113.1.7.18] to [0122.1.7.18] for the disclosure of theses paragraphs see [0113.1.7.7] to [0122.1.7.7] above

The activation of an endogenous polypeptide having above-mentioned activity, e.g. having the activity of a protein as shown in the respective line in Table II, application no. 18, column 3 or of the polypeptide of the invention, e.g. conferring the increase of the fine chemical cerotic acid, heneicosanoic acid, lignoceric acid, melissic acid or montanic acid, respectively, by increase of expression or activity in the cytoplasm, and/or in the cytosol, and/or in an organelle, such as plastids or mitochondria, can also be increased by introducing a synthetic transcription factor, which binds close to the coding region of the gene encoding the protein as shown in the respective line in Table II, application no. 18, column 5 or 8, or homologs or fragments thereof, and activates its transcription. A chimeric zinc finger protein can be constructed, which comprises a specific DNA-binding domain and an activation domain as e.g. the VP16 domain of Herpes Simplex virus. The specific binding domain can bind to the regulatory region of the gene encoding the protein as shown in the respective line in Table II, application no. 18, column 5 or 8, or homologs or fragments thereof. The expression of the chimeric transcription factor in a non-human organism, in particular in a plant, leads to a specific expression of the protein as shown in the respective line in Table II, application no. 18, column 5 or 8, or homologs or fragments thereof, see e.g. in WO01/52620, Oriz, Proc. Natl. Acad. Sci. USA 99, 13290 (2002), or Guan, Proc. Natl. Acad. Sci. USA 99, 13296 (2002).

[0124.1.7.18] to [0127.1.7.18] for the disclosure of these paragraphs see [0124.1.7.7] to [0127.1.7.7] above

Owing to the introduction of a gene or a plurality of genes conferring the expression of the nucleic acid molecule of the invention or the polypeptide of the invention, for example the nucleic acid construct mentioned below, or encoding the protein as shown in the respective line in Table II, application no. 18, column 5 or 8, or homologs or fragments thereof, into a non-human organism alone or in combination with other genes, it is possible not only to increase the biosynthetic flux towards the end product, but also to increase, modify or create de novo an advantageous, preferably novel metabolites composition in the non-human organism, e.g. an advantageous composition comprising a higher content of (from a viewpoint of nutritional physiology limited) cerotic acid, heneicosanoic acid, lignoceric acid, melissic acid or montanic acid and if desired other fatty acids, waxes e.g. VLCFA and/or lipids, and/or other metabolites, in free or bound form.

for the disclosure of this paragraph see [0129.1.7.7] above

Accordingly, in one embodiment, the process according to the invention relates to a process, which comprises:
(a) providing a non-human organism, preferably a microorganism, a plant cell, a plant tissue, a plant or a part thereof, more preferably a microorganism, a plant tissue, a plant or a part thereof;
(b) increasing the activity of a protein as shown in the respective line in Table II, application no. 18, column 3 or of a polypeptide being encoded by the respective nucleic acid molecule of the present invention and described below, e.g. conferring an increase of the fine chemical cerotic acid, heneicosanoic acid, lignoceric acid, melissic acid or montanic acid, respectively, in the non-human organism, preferably in the microorganism, the plant cell, the plant tissue, the plant or a part thereof, more preferably a microorganism, a plant tissue, a plant or a part thereof, especially cytoplasmic or in an organelle, like plastids or mitochondria,
(c) growing the non-human organism, preferably the microorganism, the plant cell, the plant tissue, the plant or a part thereof, under conditions which permit the production of said chemical in the non-human organism, preferably the microorganism, the plant cell, the plant tissue, the plant or a part thereof; and
(d) if desired, recovering, optionally isolating, said fine chemical, in free and/or bound form, and, optionally further free and/or bound fatty acids, waxes, e.g. VLCFAs, and/or other metabolites synthesized by the non-human organism, the microorganism, the plant cell, the plant tissue, the plant or a part thereof.

[0131.1.7.18] to [0133.1.7.18] for the disclosure of these paragraphs see [0131.1.7.7] to [0133.1.7.7] above

The non-human organism, in particular the microorganism, plant or part thereof, is advantageously grown in such a way that it is not only possible to recover, if desired to isolate the free or bound fine chemical or the free and bound fine chemical but as option it is also possible to produce, recover and, if desired to isolate, other free or/and bound vitamins and/or other metabolites.

The organisms are harvested in a manner known per se and appropriate for the particular organism. Microorganisms such as bacteria, mosses, yeasts and fungi or plant cells which are cultured in liquid media by fermentation may be removed, for example, by centrifugation, decanting or filtration. Plants are grown on solid media in a manner known per se and harvested accordingly.

The organisms, its cells or a part thereof or the recovered, and if desired isolated, waxes, VLCFAs, e.g. cerotic acid, heneicosanoic acid, lignoceric acid, melissic acid and/or montanic acid or derivates thereof, life salts, ethers esters, oxo and/or hydroxy derivates and/or reduced derivates like alcohols can then be processed further directly into foodstuffs or animal feeds or for other applications, for example according to the disclosures made in WO/2005/083093 or WO2005/012316, which are expressly incorporated herein by reference.

Fatty Acids are isolated from the harvested biomass in a manner known per se, for example by extraction and, where appropriate, further chemical or physical purification processes such as, for example, precipitation methods, crystallography, thermal separation methods such as rectification methods or physical separation methods such as, for example, chromatography.

Especially extraction methods using plant materials are described by Cahoon et al. (Proc. Natl. Acad. Sci. USA 96 (22), 12935 (1999)) or Browse et al., Analytic Biochemistry 152,141 (1986)).

[0135.1.7.18] to [0139.1.7.18] for the disclosure of these paragraphs see [0135.1.7.7] to [0139.1.7.7] above

In another embodiment, the invention relates to a (isolated) nucleic acid molecule, which comprises a nucleic acid molecule selected from the group consisting of:
(a) a nucleic acid molecule encoding a polypeptide shown in Table II, application no. 18, column 5 or 8, preferably shown in Table II B, application no. 18, column 8, or a homolog or a fragment thereof;
(b) a nucleic acid molecule shown in Table I, application no. 18, column 5 or 8, preferably shown in Table I B, application no. 18, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 18, preferably in column 8 of Table II B, application no. 18;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 18, preferably shown in column 8 of Table I B, application no. 18, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 18;
(i) a nucleic acid molecule encoding a polypeptide having the activity represented by a respective protein as depicted in column 5 of Table II, application no. 18;
(j) a nucleic acid molecule comprising a polynucleotide which is obtained by amplifying a cDNA library or a genomic library using the primers in Table III, application no. 18, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 18.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II application no. 18 and confers a generation or an increase of the respective fine chemical in a non-human organism or a part thereof as compared to a corresponding wild-type non-human organism or part thereof.

In another embodiment, the invention relates to a (isolated) nucleic acid molecule, which comprises a nucleic acid molecule selected from the group consisting of:
(a) a nucleic acid molecule encoding a polypeptide shown in Table II, column 5 or 8, application no. 18, preferably shown in Table II A, application no. 18, in column 5 or in Table II A, application no. 18, column 8 or in Table II B, application no. 18, column 8, or a homolog or a fragment thereof;
(b) a nucleic acid molecule shown in Table I, application no. 18, column 5 or 8, preferably shown in Table I A, application no. 18, in column 5 or in Table I A, application no. 18, column 8 or in Table I B, application no. 18, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 18, preferably shown in Table II A, application no. 18, in column 5 or in Table II A, application no. 18, column 8 or in Table II B, application no. 18, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 18, preferably shown in Table I A, application no. 18, in column 5 or in Table I A, application no. 18, column 8 or in Table I B column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 18;
(i) a nucleic acid molecule encoding a polypeptide having the activity represented by a respective protein as depicted in column 5 of Table II, application no. 18;
(j) a nucleic acid molecule comprising a polynucleotide which is obtained by amplifying a cDNA library or a genomic library using the primers in Table III, application no. 18, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt,500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto;
   whereby, preferably, the nucleic acid molecule according to (a), (b), (c), (d), (e), (f), (g), (h), (i), (j) or (k) distinguishes over the sequence depicted in Table I A and/or I B, application no. 18, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 18, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 18, column 5 or 8, or the coding region thereof, respectively. In a further embodiment the nucleic acid molecule does not encode the polypeptide sequence shown in Table II A and/or II B, application no. 18, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 18, column 5 or 8 and does not encode a protein of the sequence shown in Table II A and/or II B, application no. 18, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 18, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 18, column 5 or 8 but less than 100%, preferably less than 99.999%, 99.99%, 99.9%, 99%, 98%, 97%, 96% or 95% identical to the sequence shown in Table II A and/or II B, application no. 18, column 5 or 8.
   (All the references to Tables I to IV are references to the corresponding lines of Tables I to IV.)

In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 18.

In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II, application no. 18, and confers a generation or an increase of the respective fine chemical in a non-human organism or a part thereof as compared to a corresponding wild-type non-human organism or part thereof.

[0144.1.7.18] to [0155.1.7.18] for the disclosure of these paragraph see [0144.1.7.7] to [0155.1.7.7] above

In a preferred embodiment a nucleic acid construct, for example an expression cassette, comprises upstream, i.e. at the 5′ end of the encoding sequence, a promoter and downstream, i.e. at the 3′ end, a polyadenylation signal and optionally other regulatory elements which are operably linked to the intervening encoding sequence with one of the nucleic acids as depicted in the respective line in Table I, application no. 18, column 5 or 8, preferably the coding region thereof, or a homolog or a fragment thereof. By an operable linkage is meant the sequential arrangement of promoter, encoding sequence, terminator, optionally other regulatory elements and optionally targeting sequences in such a way that each of the regulatory elements/targeting sequence can fulfill its function in the expression of the encoding sequence in due manner. In one embodiment the sequences preferred for operable linkage are targeting sequences for ensuring subcellular localization in organelles, like plastids or mitochondria. However, targeting sequences for ensuring subcellular localization in the endoplasmic reticulum (=ER), in the nucleus, in oil corpuscles or other compartments may also be employed as well as translation promoters such as the 5′ lead sequence in tobacco mosaic virus (Gallie et al., Nucl. Acids Res. 15, 8693 (1987)).

for the disclosure of this paragraph see [0157.1.7.7] above

In an embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 18, column 5 or 8, preferably shown in Table II A, application no. 18, column 5, or in Table II A, application no. 18, column 8, or in Table II B, application no. 18, column 8, or a homolog or a fragment thereof;
   (b) a nucleic acid molecule shown in Table I, application no. 18, column 5 or 8, preferably shown in Table I A, application no. 18, column 5, or in Table I A, application no. 18, column 8, or in Table I B, application no. 18, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 18, preferably shown in Table II A, application no. 18, column 5, or in Table II A, application no. 18, column 8, or in Table II B, application no. 18, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 18, preferably shown in Table I A, application no. 18, column 5, or in Table I A, application no. 18, column 8, or in Table I B, application no. 18, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 18;
   (i) a nucleic acid molecule encoding a polypeptide having the activity represented by a respective protein as depicted in column 5 of Table II, application no. 18;
   (j) a nucleic acid molecule comprising a polynucleotide which is obtained by amplifying a cDNA library or a genomic library using the primers in Table III, application no. 18, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto.
      and, optionally
3) nucleic acid molecule encoding a transit peptide, preferably encoding a plastidal transit peptide or a mitochondrial transit peptide;
   which are operable linked.
   (All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 18.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 18 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 18, in the same line as of the respective nucleic acid molecule according to 2) “plastidic” is depicted, or encoding a mitochondrial transit peptide, especially in case in column 6 of Table I, application no. 18, in the same line as of the respective nucleic acid molecule according to 2) “mitochondric” is depicted.

In another embodiment thereof the expression cassette does not comprise 3) a nucleic acid molecule encoding a transit peptide, especially in case in column 6 of Table I, application no. 18, in the same line as of the respective nucleic acid molecule according to 2) “non-targeted” is depicted.

In a further embodiment, the invention relates to an expression cassette, comprising
1) a promoter, preferably a promoter selected from the group consisting of Big35S, PCUbi, Super and USP;
2) a nucleic acid molecule selected from the group consisting of
   (a) a nucleic acid molecule encoding a polypeptide shown in Table II, application no. 18, column 5 or 8, preferably shown in Table II A, application no. 18, column 5, or in Table II A, application no. 18, column 8, or in Table II B, application no. 18, column 8, or a homolog or a fragment thereof;
   (b) a nucleic acid molecule shown in Table I, application no. 18, column 5 or 8, preferably shown in Table I A, application no. 18, column 5, or in Table I A, application no. 18, column 8, or in Table I B, application no. 18, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 18, preferably shown in Table II A, application no. 18, column 5, or in Table II A, application no. 18, column 8, or in Table II B, application no. 18, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 18, preferably shown in Table I A, application no. 18, column 5, or in Table I A, application no. 18, column 8,or in Table I B, application no. 18, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 18;
   (i) a nucleic acid molecule encoding a polypeptide having the activity represented by a respective protein as depicted in column 5 of Table II, application no. 18;
   (j) a nucleic acid molecule comprising a polynucleotide which is obtained by amplifying a cDNA library or a genomic library using the primers in Table III, application no. 18, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, optionally
3) nucleic acid molecule encoding a transit peptide, preferably encoding a plastidal transit peptide or a mitochondrial transit peptide;
   which are operable linked and
   whereby, preferably, the nucleic acid molecule according to 2 (a), (b), (c), (d), (e), (f), (g), (h), (i), (j) or (k) distinguishes over the sequence depicted in Table I A and/or I B, application no. 18, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention according to 2) does not consist of the sequence shown in Table I A and/or I B, application no. 18, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 18, column 5 or 8, or the coding region thereof, respectively. In a further embodiment the nucleic acid molecule does not encode the polypeptide sequence shown in Table II A and/or II B, application no. 18, column 5 or 8 but homologs thereof.

Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 18, column 5 or 8 and does not encode a protein of the sequence shown in Table II A and/or II B, application no. 18, column 5 or 8. Accordingly, in one embodiment, the protein encoded by a sequence of a nucleic acid according to 2(a), (b), (c), (d), (e), (f), (g), (h), (i), (j) or (k) does not consist of the sequence shown in Table I A and/or I B, application no. 18, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 18, column 5 or 8 but less than 100%, 99.999%, 99.99%, 99.9%, 99%, 98%, 97%, 96% or 95% identical to the sequence shown in Table II A and/or II B, application no. 18, column 5 or 8.

(All the references to Tables I to IV are references to the corresponding lines of Tables I to IV.)

In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 18.

In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 18 and confers a generation or an increase of the respective fine chemical in a non-human organism or a part thereof as compared to a corresponding wild-type non-human organism or part thereof.

In another embodiment thereof the expression cassette comprises 3) a nucleic acid molecule encoding a transit peptide, preferably encoding a plastidal transit peptide, especially in case in column 6 of Table I, application no. 18, in the same line as of the respective nucleic acid molecule according to 2) “plastidic” is depicted, or encoding a mitochondrial transit peptide, especially in case in column 6 of Table I, application no. 18, in the same line as of the respective nucleic acid molecule according to 2) “mitochondric” is depicted.

In another embodiment thereof the expression cassette does not comprise 3) a nucleic acid molecule encoding a transit peptide, especially in case in column 6 of Table I, application no. 18, in the same line as of the respective nucleic acid molecule according to 2) “non-targeted” is depicted.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17968, or a homolog or fragment thereof, or a homolog thereof being depicted in Table I, application no. 18, column 8, in the same line as SEQ ID NO. 17968, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 17968 is depicted, plastidic is mentioned, or a mitochondrial transit peptide in case in Table I, application no. 18, in column 6 in the same line as SEQ ID NO. 17968 is depicted, mitochondric is mentioned; which is operable linked, too.

In another embodiment thereof the expression cassette does not comprise an additional transit peptide in case in Table I, application no. 18, in column 6 in the same line as SEQ ID NO. 17968 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 18235, or a homolog or fragment thereof, or a homolog thereof being depicted in Table I, application no. 18, column 8, in the same line as SEQ ID NO. 18235, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 18235 is depicted, plastidic is mentioned, or a mitochondrial transit peptide in case in Table I, application no. 18, in column 6 in the same line as SEQ ID NO. 18235 is depicted, mitochondric is mentioned; which is operable linked, too.

In another embodiment thereof the expression cassette does not comprise an additional transit peptide in case in Table I, application no. 18, in column 6 in the same line as SEQ ID NO. 18235 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 22249, or a homolog or fragment thereof, or a homolog thereof being depicted in Table I, application no. 18, column 8, in the same line as SEQ ID NO. 22249, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 22249 is depicted, plastidic is mentioned, or a mitochondrial transit peptide in case in Table I, application no. 18, in column 6 in the same line as SEQ ID NO. 22249 is depicted, mitochondric is mentioned; which is operable linked, too.

In another embodiment thereof the expression cassette does not comprise an additional transit peptide in case in Table I, application no. 18, in column 6 in the same line as SEQ ID NO. 22249 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 152081, or a homolog or fragment thereof, or a homolog thereof being depicted in Table I, application no. 18, column 8, in the same line as SEQ ID NO. 152081, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 152081 is depicted, plastidic is mentioned, or a mitochondrial transit peptide in case in Table I, application no. 18, in column 6 in the same line as SEQ ID NO. 152081 is depicted, mitochondric is mentioned; which is operable linked, too.

In another embodiment thereof the expression cassette does not comprise an additional transit peptide in case in Table I, application no. 18, in column 6 in the same line as SEQ ID NO. 152081 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68777, or a homolog or fragment thereof, or a homolog thereof being depicted in Table I, application no. 18, column 8, in the same line as SEQ ID NO. 68777, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 68777 is depicted, plastidic is mentioned, or a mitochondrial transit peptide in case in Table I, application no. 18, in column 6 in the same line as SEQ ID NO. 68777 is depicted, mitochondric is mentioned; which is operable linked, too.

In another embodiment thereof the expression cassette does not comprise an additional transit peptide in case in Table I, application no. 18, in column 6 in the same line as SEQ ID NO. 68777 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 2935, or a homolog or fragment thereof, or a homolog thereof being depicted in Table I, application no. 18, column 8, in the same line as SEQ ID NO. 2935, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 2935 is depicted, plastidic is mentioned, or a mitochondrial transit peptide in case in Table I, application no. 18, in column 6 in the same line as SEQ ID NO. 2935 is depicted, mitochondric is mentioned; which is operable linked, too.

In another embodiment thereof the expression cassette does not comprise an additional transit peptide in case in Table I, application no. 18, in column 6 in the same line as SEQ ID NO. 2935 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 24232, or a homolog or fragment thereof, or a homolog thereof being depicted in Table I, application no. 18, column 8, in the same line as SEQ ID NO. 24232, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 24232 is depicted, plastidic is mentioned, or a mitochondrial transit peptide in case in Table I, application no. 18, in column 6 in the same line as SEQ ID NO. 24232 is depicted, mitochondric is mentioned; which is operable linked, too.

In another embodiment thereof the expression cassette does not comprise an additional transit peptide in case in Table I, application no. 18, in column 6 in the same line as SEQ ID NO. 24232 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4348, or a homolog or fragment thereof, or a homolog thereof being depicted in Table I, application no. 18, column 8, in the same line as SEQ ID NO. 4348, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 4348 is depicted, plastidic is mentioned, or a mitochondrial transit peptide in case in Table I, application no. 18, in column 6 in the same line as SEQ ID NO. 4348 is depicted, mitochondric is mentioned; which is operable linked, too.

In another embodiment thereof the expression cassette does not comprise an additional transit peptide in case in Table I, application no. 18, in column 6 in the same line as SEQ ID NO. 4348 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 24492, or a homolog or fragment thereof, or a homolog thereof being depicted in Table I, application no. 18, column 8, in the same line as SEQ ID NO. 24492, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 24492 is depicted, plastidic is mentioned, or a mitochondrial transit peptide in case in Table I, application no. 18, in column 6 in the same line as SEQ ID NO. 24492 is depicted, mitochondric is mentioned; which is operable linked, too.

In another embodiment thereof the expression cassette does not comprise an additional transit peptide in case in Table I, application no. 18, in column 6 in the same line as SEQ ID NO. 24492 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 5493, or a homolog or fragment thereof, or a homolog thereof being depicted in Table I, application no. 18, column 8, in the same line as SEQ ID NO. 5493, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 5493 is depicted, plastidic is mentioned, or a mitochondrial transit peptide in case in Table I, application no. 18, in column 6 in the same line as SEQ ID NO. 5493 is depicted, mitochondric is mentioned; which is operable linked, too.

In another embodiment thereof the expression cassette does not comprise an additional transit peptide in case in Table I, application no. 18, in column 6 in the same line as SEQ ID NO. 5493 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 31926, or a homolog or fragment thereof, or a homolog thereof being depicted in Table I, application no. 18, column 8, in the same line as SEQ ID NO. 31926, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 31926 is depicted, plastidic is mentioned, or a mitochondrial transit peptide in case in Table I, application no. 18, in column 6 in the same line as SEQ ID NO. 31926 is depicted, mitochondric is mentioned; which is operable linked, too.

In another embodiment thereof the expression cassette does not comprise an additional transit peptide in case in Table I, application no. 18, in column 6 in the same line as SEQ ID NO. 31926 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6510, or a homolog or fragment thereof, or a homolog thereof being depicted in Table I, application no. 18, column 8, in the same line as SEQ ID NO. 6510, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 6510 is depicted, plastidic is mentioned, or a mitochondrial transit peptide in case in Table I, application no. 18, in column 6 in the same line as SEQ ID NO. 6510 is depicted, mitochondric is mentioned; which is operable linked, too.

In another embodiment thereof the expression cassette does not comprise an additional transit peptide in case in Table I, application no. 18, in column 6 in the same line as SEQ ID NO. 6510 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 32037, or a homolog or fragment thereof, or a homolog thereof being depicted in Table I, application no. 18, column 8, in the same line as SEQ ID NO. 32037, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 32037 is depicted, plastidic is mentioned, or a mitochondrial transit peptide in case in Table I, application no. 18, in column 6 in the same line as SEQ ID NO. 32037 is depicted, mitochondric is mentioned; which is operable linked, too.

In another embodiment thereof the expression cassette does not comprise an additional transit peptide in case in Table I, application no. 18, in column 6 in the same line as SEQ ID NO. 32037 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 33457, or a homolog or fragment thereof, or a homolog thereof being depicted in Table I, application no. 18, column 8, in the same line as SEQ ID NO. 33457, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 33457 is depicted, plastidic is mentioned, or a mitochondrial transit peptide in case in Table I, application no. 18, in column 6 in the same line as SEQ ID NO. 33457 is depicted, mitochondric is mentioned; which is operable linked, too.

In another embodiment thereof the expression cassette does not comprise an additional transit peptide in case in Table I, application no. 18, in column 6 in the same line as SEQ ID NO. 33457 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 33596, or a homolog or fragment thereof, or a homolog thereof being depicted in Table I, application no. 18, column 8, in the same line as SEQ ID NO. 33596, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 33596 is depicted, plastidic is mentioned, or a mitochondrial transit peptide in case in Table I, application no. 18, in column 6 in the same line as SEQ ID NO. 33596 is depicted, mitochondric is mentioned; which is operable linked, too.

In another embodiment thereof the expression cassette does not comprise an additional transit peptide in case in Table I, application no. 18, in column 6 in the same line as SEQ ID NO. 33596 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 34044, or a homolog or fragment thereof, or a homolog thereof being depicted in Table I, application no. 18, column 8, in the same line as SEQ ID NO. 34044, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 34044 is depicted, plastidic is mentioned, or a mitochondrial transit peptide in case in Table I, application no. 18, in column 6 in the same line as SEQ ID NO. 34044 is depicted, mitochondric is mentioned; which is operable linked, too.

In another embodiment thereof the expression cassette does not comprise an additional transit peptide in case in Table I, application no. 18, in column 6 in the same line as SEQ ID NO. 34044 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 34889, or a homolog or fragment thereof, or a homolog thereof being depicted in Table I, application no. 18, column 8, in the same line as SEQ ID NO. 34889, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 34889 is depicted, plastidic is mentioned, or a mitochondrial transit peptide in case in Table I, application no. 18, in column 6 in the same line as SEQ ID NO. 34889 is depicted, mitochondric is mentioned; which is operable linked, too.

In another embodiment thereof the expression cassette does not comprise an additional transit peptide in case in Table I, application no. 18, in column 6 in the same line as SEQ ID NO. 34889 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7081, or a homolog or fragment thereof, or a homolog thereof being depicted in Table I, application no. 18, column 8, in the same line as SEQ ID NO. 7081, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 7081 is depicted, plastidic is mentioned, or a mitochondrial transit peptide in case in Table I, application no. 18, in column 6 in the same line as SEQ ID NO. 7081 is depicted, mitochondric is mentioned; which is operable linked, too.

In another embodiment thereof the expression cassette does not comprise an additional transit peptide in case in Table I, application no. 18, in column 6 in the same line as SEQ ID NO. 7081 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7333, or a homolog or fragment thereof, or a homolog thereof being depicted in Table I, application no. 18, column 8, in the same line as SEQ ID NO. 7333, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 7333 is depicted, plastidic is mentioned, or a mitochondrial transit peptide in case in Table I, application no. 18, in column 6 in the same line as SEQ ID NO. 7333 is depicted, mitochondric is mentioned; which is operable linked, too.

In another embodiment thereof the expression cassette does not comprise an additional transit peptide in case in Table I, application no. 18, in column 6 in the same line as SEQ ID NO. 7333 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 35967, or a homolog or fragment thereof, or a homolog thereof being depicted in Table I, application no. 18, column 8, in the same line as SEQ ID NO. 35967, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 35967 is depicted, plastidic is mentioned, or a mitochondrial transit peptide in case in Table I, application no. 18, in column 6 in the same line as SEQ ID NO. 35967 is depicted, mitochondric is mentioned; which is operable linked, too.

In another embodiment thereof the expression cassette does not comprise an additional transit peptide in case in Table I, application no. 18, in column 6 in the same line as SEQ ID NO. 35967 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 36489, or a homolog or fragment thereof, or a homolog thereof being depicted in Table I, application no. 18, column 8, in the same line as SEQ ID NO. 36489, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 36489 is depicted, plastidic is mentioned, or a mitochondrial transit peptide in case in Table I, application no. 18, in column 6 in the same line as SEQ ID NO. 36489 is depicted, mitochondric is mentioned; which is operable linked, too.

In another embodiment thereof the expression cassette does not comprise an additional transit peptide in case in Table I, application no. 18, in column 6 in the same line as SEQ ID NO. 36489 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7917, or a homolog or fragment thereof, or a homolog thereof being depicted in Table I, application no. 18, column 8, in the same line as SEQ ID NO. 7917, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 7917 is depicted, plastidic is mentioned, or a mitochondrial transit peptide in case in Table I, application no. 18, in column 6 in the same line as SEQ ID NO. 7917 is depicted, mitochondric is mentioned; which is operable linked, too.

In another embodiment thereof the expression cassette does not comprise an additional transit peptide in case in Table I, application no. 18, in column 6 in the same line as SEQ ID NO. 7917 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 76032, or a homolog or fragment thereof, or a homolog thereof being depicted in Table I, application no. 18, column 8, in the same line as SEQ ID NO. 76032, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 76032 is depicted, plastidic is mentioned, or a mitochondrial transit peptide in case in Table I, application no. 18, in column 6 in the same line as SEQ ID NO. 76032 is depicted, mitochondric is mentioned; which is operable linked, too.

In another embodiment thereof the expression cassette does not comprise an additional transit peptide in case in Table I, application no. 18, in column 6 in the same line as SEQ ID NO. 76032 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 37400, or a homolog or fragment thereof, or a homolog thereof being depicted in Table I, application no. 18, column 8, in the same line as SEQ ID NO. 37400, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 37400 is depicted, plastidic is mentioned, or a mitochondrial transit peptide in case in Table I, application no. 18, in column 6 in the same line as SEQ ID NO. 37400 is depicted, mitochondric is mentioned; which is operable linked, too.

In another embodiment thereof the expression cassette does not comprise an additional transit peptide in case in Table I, application no. 18, in column 6 in the same line as SEQ ID NO. 37400 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 98778, or a homolog or fragment thereof, or a homolog thereof being depicted in Table I, application no. 18, column 8, in the same line as SEQ ID NO. 98778, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 98778 is depicted, plastidic is mentioned, or a mitochondrial transit peptide in case in Table I, application no. 18, in column 6 in the same line as SEQ ID NO. 98778 is depicted, mitochondric is mentioned; which is operable linked, too.

In another embodiment thereof the expression cassette does not comprise an additional transit peptide in case in Table I, application no. 18, in column 6 in the same line as SEQ ID NO. 98778 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7947, or a homolog or fragment thereof, or a homolog thereof being depicted in Table I, application no. 18, column 8, in the same line as SEQ ID NO. 7947, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 7947 is depicted, plastidic is mentioned, or a mitochondrial transit peptide in case in Table I, application no. 18, in column 6 in the same line as SEQ ID NO. 7947 is depicted, mitochondric is mentioned; which is operable linked, too.

In another embodiment thereof the expression cassette does not comprise an additional transit peptide in case in Table I, application no. 18, in column 6 in the same line as SEQ ID NO. 7947 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38300, or a homolog or fragment thereof, or a homolog thereof being depicted in Table I, application no. 18, column 8, in the same line as SEQ ID NO. 38300, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 38300 is depicted, plastidic is mentioned, or a mitochondrial transit peptide in case in Table I, application no. 18, in column 6 in the same line as SEQ ID NO. 38300 is depicted, mitochondric is mentioned; which is operable linked, too.

In another embodiment thereof the expression cassette does not comprise an additional transit peptide in case in Table I, application no. 18, in column 6 in the same line as SEQ ID NO. 38300 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 79217, or a homolog or fragment thereof, or a homolog thereof being depicted in Table I, application no. 18, column 8, in the same line as SEQ ID NO. 79217, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 79217 is depicted, plastidic is mentioned, or a mitochondrial transit peptide in case in Table I, application no. 18, in column 6 in the same line as SEQ ID NO. 79217 is depicted, mitochondric is mentioned; which is operable linked, too.

In another embodiment thereof the expression cassette does not comprise an additional transit peptide in case in Table I, application no. 18, in column 6 in the same line as SEQ ID NO. 79217 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 79509, or a homolog or fragment thereof, or a homolog thereof being depicted in Table I, application no. 18, column 8, in the same line as SEQ ID NO. 79509, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 79509 is depicted, plastidic is mentioned, or a mitochondrial transit peptide in case in Table I, application no. 18, in column 6 in the same line as SEQ ID NO. 79509 is depicted, mitochondric is mentioned; which is operable linked, too.

In another embodiment thereof the expression cassette does not comprise an additional transit peptide in case in Table I, application no. 18, in column 6 in the same line as SEQ ID NO. 79509 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40383, or a homolog or fragment thereof, or a homolog thereof being depicted in Table I, application no. 18, column 8, in the same line as SEQ ID NO. 40383, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 40383 is depicted, plastidic is mentioned, or a mitochondrial transit peptide in case in Table I, application no. 18, in column 6 in the same line as SEQ ID NO. 40383 is depicted, mitochondric is mentioned; which is operable linked, too.

In another embodiment thereof the expression cassette does not comprise an additional transit peptide in case in Table I, application no. 18, in column 6 in the same line as SEQ ID NO. 40383 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 82424, or a homolog or fragment thereof, or a homolog thereof being depicted in Table I, application no. 18, column 8, in the same line as SEQ ID NO. 82424, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 82424 is depicted, plastidic is mentioned, or a mitochondrial transit peptide in case in Table I, application no. 18, in column 6 in the same line as SEQ ID NO. 82424 is depicted, mitochondric is mentioned; which is operable linked, too.

In another embodiment thereof the expression cassette does not comprise an additional transit peptide in case in Table I, application no. 18, in column 6 in the same line as SEQ ID NO. 82424 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 125487, or a homolog or fragment thereof, or a homolog thereof being depicted in Table I, application no. 18, column 8, in the same line as SEQ ID NO. 125487, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 125487 is depicted, plastidic is mentioned, or a mitochondrial transit peptide in case in Table I, application no. 18, in column 6 in the same line as SEQ ID NO. 125487 is depicted, mitochondric is mentioned; which is operable linked, too.

In another embodiment thereof the expression cassette does not comprise an additional transit peptide in case in Table I, application no. 18, in column 6 in the same line as SEQ ID NO. 125487 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 10740, or a homolog or fragment thereof, or a homolog thereof being depicted in Table I, application no. 18, column 8, in the same line as SEQ ID NO. 10740, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 10740 is depicted, plastidic is mentioned, or a mitochondrial transit peptide in case in Table I, application no. 18, in column 6 in the same line as SEQ ID NO. 10740 is depicted, mitochondric is mentioned; which is operable linked, too.

In another embodiment thereof the expression cassette does not comprise an additional transit peptide in case in Table I, application no. 18, in column 6 in the same line as SEQ ID NO. 10740 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 45321, or a homolog or fragment thereof, or a homolog thereof being depicted in Table I, application no. 18, column 8, in the same line as SEQ ID NO. 45321, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 45321 is depicted, plastidic is mentioned, or a mitochondrial transit peptide in case in Table I, application no. 18, in column 6 in the same line as SEQ ID NO. 45321 is depicted, mitochondric is mentioned; which is operable linked, too.

In another embodiment thereof the expression cassette does not comprise an additional transit peptide in case in Table I, application no. 18, in column 6 in the same line as SEQ ID NO. 45321 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 10811, or a homolog or fragment thereof, or a homolog thereof being depicted in Table I, application no. 18, column 8, in the same line as SEQ ID NO. 10811, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 10811 is depicted, plastidic is mentioned, or a mitochondrial transit peptide in case in Table I, application no. 18, in column 6 in the same line as SEQ ID NO. 10811 is depicted, mitochondric is mentioned; which is operable linked, too.

In another embodiment thereof the expression cassette does not comprise an additional transit peptide in case in Table I, application no. 18, in column 6 in the same line as SEQ ID NO. 10811 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 46850, or a homolog or fragment thereof, or a homolog thereof being depicted in Table I, application no. 18, column 8, in the same line as SEQ ID NO. 46850, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 46850 is depicted, plastidic is mentioned, or a mitochondrial transit peptide in case in Table I, application no. 18, in column 6 in the same line as SEQ ID NO. 46850 is depicted, mitochondric is mentioned; which is operable linked, too.

In another embodiment thereof the expression cassette does not comprise an additional transit peptide in case in Table I, application no. 18, in column 6 in the same line as SEQ ID NO. 46850 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 47526, or a homolog or fragment thereof, or a homolog thereof being depicted in Table I, application no. 18, column 8, in the same line as SEQ ID NO. 47526, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 47526 is depicted, plastidic is mentioned, or a mitochondrial transit peptide in case in Table I, application no. 18, in column 6 in the same line as SEQ ID NO. 47526 is depicted, mitochondric is mentioned; which is operable linked, too.

In another embodiment thereof the expression cassette does not comprise an additional transit peptide in case in Table I, application no. 18, in column 6 in the same line as SEQ ID NO. 47526 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 84265, or a homolog or fragment thereof, or a homolog thereof being depicted in Table I, application no. 18, column 8, in the same line as SEQ ID NO. 84265, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 84265 is depicted, plastidic is mentioned, or a mitochondrial transit peptide in case in Table I, application no. 18, in column 6 in the same line as SEQ ID NO. 84265 is depicted, mitochondric is mentioned; which is operable linked, too.

In another embodiment thereof the expression cassette does not comprise an additional transit peptide in case in Table I, application no. 18, in column 6 in the same line as SEQ ID NO. 84265 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 84869, or a homolog or fragment thereof, or a homolog thereof being depicted in Table I, application no. 18, column 8, in the same line as SEQ ID NO. 84869, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 84869 is depicted, plastidic is mentioned, or a mitochondrial transit peptide in case in Table I, application no. 18, in column 6 in the same line as SEQ ID NO. 84869 is depicted, mitochondric is mentioned; which is operable linked, too.

In another embodiment thereof the expression cassette does not comprise an additional transit peptide in case in Table I, application no. 18, in column 6 in the same line as SEQ ID NO. 84869 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 101707, or a homolog or fragment thereof, or a homolog thereof being depicted in Table I, application no. 18, column 8, in the same line as SEQ ID NO. 101707, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 101707 is depicted, plastidic is mentioned, or a mitochondrial transit peptide in case in Table I, application no. 18, in column 6 in the same line as SEQ ID NO. 101707 is depicted, mitochondric is mentioned; which is operable linked, too.

In another embodiment thereof the expression cassette does not comprise an additional transit peptide in case in Table I, application no. 18, in column 6 in the same line as SEQ ID NO. 101707 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 51268, or a homolog or fragment thereof, or a homolog thereof being depicted in Table I, application no. 18, column 8, in the same line as SEQ ID NO. 51268, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 51268 is depicted, plastidic is mentioned, or a mitochondrial transit peptide in case in Table I, application no. 18, in column 6 in the same line as SEQ ID NO. 51268 is depicted, mitochondric is mentioned; which is operable linked, too.

In another embodiment thereof the expression cassette does not comprise an additional transit peptide in case in Table I, application no. 18, in column 6 in the same line as SEQ ID NO. 51268 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 85743, or a homolog or fragment thereof, or a homolog thereof being depicted in Table I, application no. 18, column 8, in the same line as SEQ ID NO. 85743, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 85743 is depicted, plastidic is mentioned, or a mitochondrial transit peptide in case in Table I, application no. 18, in column 6 in the same line as SEQ ID NO. 85743 is depicted, mitochondric is mentioned; which is operable linked, too.

In another embodiment thereof the expression cassette does not comprise an additional transit peptide in case in Table I, application no. 18, in column 6 in the same line as SEQ ID NO. 85743 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 86447, or a homolog or fragment thereof, or a homolog thereof being depicted in Table I, application no. 18, column 8, in the same line as SEQ ID NO. 86447, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 86447 is depicted, plastidic is mentioned, or a mitochondrial transit peptide in case in Table I, application no. 18, in column 6 in the same line as SEQ ID NO. 86447 is depicted, mitochondric is mentioned; which is operable linked, too.

In another embodiment thereof the expression cassette does not comprise an additional transit peptide in case in Table I, application no. 18, in column 6 in the same line as SEQ ID NO. 86447 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 95744, or a homolog or fragment thereof, or a homolog thereof being depicted in Table I, application no. 18, column 8, in the same line as SEQ ID NO. 95744, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 95744 is depicted, plastidic is mentioned, or a mitochondrial transit peptide in case in Table I, application no. 18, in column 6 in the same line as SEQ ID NO. 95744 is depicted, mitochondric is mentioned; which is operable linked, too.

In another embodiment thereof the expression cassette does not comprise an additional transit peptide in case in Table I, application no. 18, in column 6 in the same line as SEQ ID NO. 95744 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 57235, or a homolog or fragment thereof, or a homolog thereof being depicted in Table I, application no. 18, column 8, in the same line as SEQ ID NO. 57235, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 57235 is depicted, plastidic is mentioned, or a mitochondrial transit peptide in case in Table I, application no. 18, in column 6 in the same line as SEQ ID NO. 57235 is depicted, mitochondric is mentioned; which is operable linked, too.

In another embodiment thereof the expression cassette does not comprise an additional transit peptide in case in Table I, application no. 18, in column 6 in the same line as SEQ ID NO. 57235 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 87655, or a homolog or fragment thereof, or a homolog thereof being depicted in Table I, application no. 18, column 8, in the same line as SEQ ID NO. 87655, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 87655 is depicted, plastidic is mentioned, or a mitochondrial transit peptide in case in Table I, application no. 18, in column 6 in the same line as SEQ ID NO. 87655 is depicted, mitochondric is mentioned; which is operable linked, too.

In another embodiment thereof the expression cassette does not comprise an additional transit peptide in case in Table I, application no. 18, in column 6 in the same line as SEQ ID NO. 87655 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 152275, or a homolog or fragment thereof, or a homolog thereof being depicted in Table I, application no. 18, column 8, in the same line as SEQ ID NO. 152275, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 152275 is depicted, plastidic is mentioned, or a mitochondrial transit peptide in case in Table I, application no. 18, in column 6 in the same line as SEQ ID NO. 152275 is depicted, mitochondric is mentioned; which is operable linked, too.

In another embodiment thereof the expression cassette does not comprise an additional transit peptide in case in Table I, application no. 18, in column 6 in the same line as SEQ ID NO. 152275 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12698, or a homolog or fragment thereof, or a homolog thereof being depicted in Table I, application no. 18, column 8, in the same line as SEQ ID NO. 12698, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 12698 is depicted, plastidic is mentioned, or a mitochondrial transit peptide in case in Table I, application no. 18, in column 6 in the same line as SEQ ID NO. 12698 is depicted, mitochondric is mentioned; which is operable linked, too.

In another embodiment thereof the expression cassette does not comprise an additional transit peptide in case in Table I, application no. 18, in column 6 in the same line as SEQ ID NO. 12698 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 61532, or a homolog or fragment thereof, or a homolog thereof being depicted in Table I, application no. 18, column 8, in the same line as SEQ ID NO. 61532, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 61532 is depicted, plastidic is mentioned, or a mitochondrial transit peptide in case in Table I, application no. 18, in column 6 in the same line as SEQ ID NO. 61532 is depicted, mitochondric is mentioned; which is operable linked, too.

In another embodiment thereof the expression cassette does not comprise an additional transit peptide in case in Table I, application no. 18, in column 6 in the same line as SEQ ID NO. 61532 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 62079, or a homolog or fragment thereof, or a homolog thereof being depicted in Table I, application no. 18, column 8, in the same line as SEQ ID NO. 62079, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 62079 is depicted, plastidic is mentioned, or a mitochondrial transit peptide in case in Table I, application no. 18, in column 6 in the same line as SEQ ID NO. 62079 is depicted, mitochondric is mentioned; which is operable linked, too.

In another embodiment thereof the expression cassette does not comprise an additional transit peptide in case in Table I, application no. 18, in column 6 in the same line as SEQ ID NO. 62079 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 121908, or a homolog or fragment thereof, or a homolog thereof being depicted in Table I, application no. 18, column 8, in the same line as SEQ ID NO. 121908, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 121908 is depicted, plastidic is mentioned, or a mitochondrial transit peptide in case in Table I, application no. 18, in column 6 in the same line as SEQ ID NO. 121908 is depicted, mitochondric is mentioned; which is operable linked, too.

In another embodiment thereof the expression cassette does not comprise an additional transit peptide in case in Table I, application no. 18, in column 6 in the same line as SEQ ID NO. 121908 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 63665, or a homolog or fragment thereof, or a homolog thereof being depicted in Table I, application no. 18, column 8, in the same line as SEQ ID NO. 63665, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 63665 is depicted, plastidic is mentioned, or a mitochondrial transit peptide in case in Table I, application no. 18, in column 6 in the same line as SEQ ID NO. 63665 is depicted, mitochondric is mentioned; which is operable linked, too.

In another embodiment thereof the expression cassette does not comprise an additional transit peptide in case in Table I, application no. 18, in column 6 in the same line as SEQ ID NO. 63665 is depicted, non-targeted is mentioned.

In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 149632, or a homolog or fragment thereof, or a homolog thereof being depicted in Table I, application no. 18, column 8, in the same line as SEQ ID NO. 149632, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 149632 is depicted, plastidic is mentioned, or a mitochondrial transit peptide in case in Table I, application no. 18, in column 6 in the same line as SEQ ID NO. 149632 is depicted, mitochondric is mentioned; which is operable linked, too.

In another embodiment thereof the expression cassette does not comprise an additional transit peptide in case in Table I, application no. 18, in column 6 in the same line as SEQ ID NO. 149632 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 14843, or a homolog or fragment thereof, or a homolog thereof being depicted in Table I, application no. 18, column 8, in the same line as SEQ ID NO. 14843, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 14843 is depicted, plastidic is mentioned, or a mitochondrial transit peptide in case in Table I, application no. 18, in column 6 in the same line as SEQ ID NO. 14843 is depicted, mitochondric is mentioned; which is operable linked, too.

In another embodiment thereof the expression cassette does not comprise an additional transit peptide in case in Table I, application no. 18, in column 6 in the same line as SEQ ID NO. 14843 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 152382, or a homolog or fragment thereof, or a homolog thereof being depicted in Table I, application no. 18, column 8, in the same line as SEQ ID NO. 152382, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 152382 is depicted, plastidic is mentioned, or a mitochondrial transit peptide in case in Table I, application no. 18, in column 6 in the same line as SEQ ID NO. 152382 is depicted, mitochondric is mentioned; which is operable linked, too.

In another embodiment thereof the expression cassette does not comprise an additional transit peptide in case in Table I, application no. 18, in column 6 in the same line as SEQ ID NO. 152382 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 66695, or a homolog or fragment thereof, or a homolog thereof being depicted in Table I, application no. 18, column 8, in the same line as SEQ ID NO. 66695, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 66695 is depicted, plastidic is mentioned, or a mitochondrial transit peptide in case in Table I, application no. 18, in column 6 in the same line as SEQ ID NO. 66695 is depicted, mitochondric is mentioned; which is operable linked, too.

In another embodiment thereof the expression cassette does not comprise an additional transit peptide in case in Table I, application no. 18, in column 6 in the same line as SEQ ID NO. 66695 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 152424, or a homolog or fragment thereof, or a homolog thereof being depicted in Table I, application no. 18, column 8, in the same line as SEQ ID NO. 152424, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 152424 is depicted, plastidic is mentioned, or a mitochondrial transit peptide in case in Table I, application no. 18, in column 6 in the same line as SEQ ID NO. 152424 is depicted, mitochondric is mentioned; which is operable linked, too.

In another embodiment thereof the expression cassette does not comprise an additional transit peptide in case in Table I, application no. 18, in column 6 in the same line as SEQ ID NO. 152424 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 92274, or a homolog or fragment thereof, or a homolog thereof being depicted in Table I, application no. 18, column 8, in the same line as SEQ ID NO. 92274, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 92274 is depicted, plastidic is mentioned, or a mitochondrial transit peptide in case in Table I, application no. 18, in column 6 in the same line as SEQ ID NO. 92274 is depicted, mitochondric is mentioned; which is operable linked, too.

In another embodiment thereof the expression cassette does not comprise an additional transit peptide in case in Table I, application no. 18, in column 6 in the same line as SEQ ID NO. 92274 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 152460, or a homolog or fragment thereof, or a homolog thereof being depicted in Table I, application no. 18, column 8, in the same line as SEQ ID NO. 152460, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 152460 is depicted, plastidic is mentioned, or a mitochondrial transit peptide in case in Table I, application no. 18, in column 6 in the same line as SEQ ID NO. 152460 is depicted, mitochondric is mentioned; which is operable linked, too.

In another embodiment thereof the expression cassette does not comprise an additional transit peptide in case in Table I, application no. 18, in column 6 in the same line as SEQ ID NO. 152460 is depicted, non-targeted is mentioned.

In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 152341, or a homolog or fragment thereof, or a homolog thereof being depicted in Table I, application no. 18, column 8, in the same line as SEQ ID NO. 152341, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 152341 is depicted, plastidic is mentioned, or a mitochondrial transit peptide in case in Table I, application no. 18, in column 6 in the same line as SEQ ID NO. 152341 is depicted, mitochondric is mentioned; which is operable linked, too.

In another embodiment thereof the expression cassette does not comprise an additional transit peptide in case in Table I, application no. 18, in column 6 in the same line as SEQ ID NO. 152341 is depicted, non-targeted is mentioned.

In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 125348, or a homolog or fragment thereof, or a homolog thereof being depicted in Table I, application no. 18, column 8, in the same line as SEQ ID NO. 125348, preferably the coding region thereof, a homolog or a fragment thereof; which are operable linked.

In an embodiment thereof the expression cassette comprises in addition c) a nucleic acid molecule encoding a transit peptide, especially a plastidal transit petide in case in Table I, application no. 18, in column 6 in the same line as SEQ ID NO. 125348 is depicted, plastidic is mentioned, or a mitochondrial transit peptide in case in Table I, application no. 18, in column 6 in the same line as SEQ ID NO. 125348 is depicted, mitochondric is mentioned; which is operable linked, too.

In another embodiment thereof the expression cassette does not comprise an additional transit peptide in case in Table I, application no. 18, in column 6 in the same line as SEQ ID NO. 125348 is depicted, non-targeted is mentioned.

[0165.1.7.18] to [0170.1.7.18] for the disclosure of these paragraphs see [0165.1.7.7] to [0170.1.7.7] above

The invention further provides a vector, comprising a nucleic acid molecule comprising 1) a nucleic acid molecule selected from the group consisting of:
(a) a nucleic acid molecule encoding a polypeptide shown in Table II, column 5 or 8, application no. 18, preferably shown in Table II A, application no. 18, in column 5, or in Table II A, application no. 18, column 8, or in Table II B, application no. 18, column 8, or a homolog or a fragment thereof;
(b) a nucleic acid molecule shown in Table I, application no. 18, column 5 or 8, preferably shown in Table I A, application no. 18, in column 5, or in Table I A, application no. 18, column 8, or in Table I B, application no. 18, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 18, preferably shown in Table II A, application no. 18, in column 5, or in Table II A, application no. 18, column 8, or in Table II B, application no. 18, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 18, preferably shown in Table I A, application no. 18, in column 5, or in Table I A, application no. 18, column 8, or in Table I B, application no. 18, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 18;
(i) a nucleic acid molecule encoding a polypeptide having the activity represented by a protein as depicted in column 5 of Table II, application no. 18;
(j) a nucleic acid molecule comprising a polynucleotide which is obtained by amplifying a cDNA library or a genomic library using the primers in Table III, application no. 18, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 18.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 18, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 18.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 18, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 18, in the same line as of the respective nucleic acid molecule according to 1) “plastidic” is depicted, or encoding a mitochondrial transit peptide, especially in case in column 6 of Table I, application no. 18, in the same line as of the respective nucleic acid molecule according to 1) “mitochondric” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 18, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 18, in the same line as of the respective nucleic acid molecule according to 1) “plastidic” is depicted, or encoding a mitochondrial transit peptide, especially in case in column 6 of Table I, application no. 18, in the same line as of the respective nucleic acid molecule according to 1) “mitochondric” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operable linked; a nucleic acid molecule encoding a transit peptide, especially in case in column 6 of Table I, application no. 18, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 18, in the same line as of the respective nucleic acid molecule according to 1) “plastidic” is depicted, or encoding a mitochondrial transit peptide, especially in case in column 6 of Table I, application no. 18, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 18 (in the same line).

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 18, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 18.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 18, in the same line as of the respective nucleic acid molecule according to 1) “plastidic” is depicted, or encoding a mitochondrial transit peptide, especially in case in column 6 of Table I, application no. 18, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 18, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 18, in the same line as of the respective nucleic acid molecule according to 1) “plastidic” is depicted, or encoding a mitochondrial transit peptide, especially in case in column 6 of Table I, application no. 18, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 18 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 18, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 18 (in the same line) and confers a generation or an increase of the respective fine chemical in a non-human organism or a part thereof as compared to a corresponding wild-type non-human organism or part thereof.

In a preferred embodiment of said vector said nucleic acid molecule according to 1 (a), (b), (c), (d), (e), (f), (g), (h), (i), (j) or (k), said promoter and optionally said nucleic acid molecule encoding a transit peptide are encompassed in an expression cassette. Accordingly in an embodiment the invention provides a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

The invention further provides a vector comprising a nucleic acid molecule comprising 1) a nucleic acid molecule selected from the group consisting of:
(a) a nucleic acid molecule encoding a polypeptide shown in Table II, column 5 or 8, application no. 18, preferably shown in Table II A, application no. 18, in column 5, or in Table II A, application no. 18, column 8, or in Table II B, application no. 18, column 8, or a homolog or a fragment thereof;
(b) a nucleic acid molecule shown in Table I, application no. 18, column 5 or 8, preferably shown in Table I A, application no. 18, in column 5, or in Table I A, application no. 18, column 8, or in Table I B, application no. 18, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 18, preferably shown in Table II A, application no. 18, in column 5, or in Table II A, application no. 18, column 8, or in Table II B, application no. 18, column 8;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 18, preferably shown in Table I A, application no. 18, in column 5, or in Table I A, application no. 18, column 8, or in Table I B, application no. 18, column 8, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
(f) a nucleic acid molecule which hybridizes with a nucleic acid molecule of (a), (b), (c), (d), or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 18;
(i) a nucleic acid molecule encoding a polypeptide having the activity represented by a protein as depicted in column 5 of Table II, application no. 18;
(j) a nucleic acid molecule comprising a polynucleotide which is obtained by amplifying a cDNA library or a genomic library using the primers in Table III, application no. 18, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
or a nucleic acid molecule comprising a sequence which is complementary thereto;
   whereby, preferably, the nucleic acid molecule according to 1 (a), (b), (c), (d), (e), (f), (g), (h), (i), (j) or (k) distinguishes over the sequence depicted in Table I A and/or I B, application no. 18, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 18, column 5 or 8, or the coding region thereof, respectively. In another embodiment, the nucleic acid molecule of the present invention, preferably the coding region thereof, is 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 18, column 5 or 8, or the coding region thereof, respectively. In a further embodiment the nucleic acid molecule does not encode the polypeptide sequence shown in Table II A and/or II B, application no. 18, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 18, column 5 or 8 and does not encode a protein of the sequence shown in Table II A and/or II B, application no. 18, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 18, column 5 and 8, or the coding region thereof. In a further embodiment, the protein of the present invention is at least 30%,40%, 50%, 60%, 70%, 80%, 90%, 95% identical to protein sequence depicted in Table II A and/or II B, application no. 18, column 5 or 8 but less than 100%, preferably less than 99.999%, 99.99% or 99.9%, more preferably less than 99%, 985, 97%, 96% or 95% identical to the sequence shown in Table II A and/or II B, application no. 18, column 5 or 8.
   (All the references to Tables I to IV are references to the corresponding lines of Tables I to IV.)

In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 18.

In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 18, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 18.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 18, and confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 18, in the same line as of the respective nucleic acid molecule according to 1) “plastidic” is depicted, or encoding a mitochondrial transit peptide, especially in case in column 6 of Table I, application no. 18, in the same line as of the respective nucleic acid molecule according to 1) “mitochondric” is depicted, which are operable linked.

In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 18, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 18, in the same line as of the respective nucleic acid molecule according to 1) “plastidic” is depicted, or encoding a mitochondrial transit peptide, especially in case in column 6 of Table I, application no. 18, in the same line as of the respective nucleic acid molecule according to 1) “mitochondric” is depicted, which are operable linked.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 18, in the same line as of the respective nucleic acid molecule according to 1) “non-targeted” is depicted.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 18, in the same line as of the respective nucleic acid molecule according to 1) “plastidic” is depicted, or encoding a mitochondrial transit peptide, especially in case in column 6 of Table I, application no. 18, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 18.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 18, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 18.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 18, in the same line as of the respective nucleic acid molecule according to 1) “plastidic” is depicted, or encoding a mitochondrial transit peptide, especially in case in column 6 of Table I, application no. 18, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 18, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 18, in the same line as of the respective nucleic acid molecule according to 1) “plastidic” is depicted, or encoding a mitochondrial transit peptide, especially in case in column 6 of Table I, application no. 18, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 18 and confers a generation or an increase of the respective fine chemical in a non-human organism or a part thereof as compared to a corresponding wild-type non-human organism or part thereof.

In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 18, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 18 and confers a generation or an increase of the respective fine chemical in a non-human organism or a part thereof as compared to a corresponding wild-type non-human organism or part thereof.

[0181.1.7.18] to [0209.1.7.18] for the disclosure of these paragraphs see [0181.1.7.7] to [0209.1.7.7] above

The genes of the invention, coding in one embodiment for an activity selected from the group consisting of 2,3-dihydroxy-2,3-dihydrophenylpropionatedehydrogenase, ABC metal ion transporter substrate-binding protein, acetyl-coenzyme A synthetase, acetyltransferase, acid shock protein, acyl transferase, agmatinase, aminotransferase, ATP-binding component of a transport system, b1522-protein, b3083-protein, b3644-protein, b3812-protein, b4029-protein, binding-protein-dependent transport systems inner membrane component, calcium-dependent protein kinase, cardiolipin synthase, chaperone protein CIpB, cyclin D, cystathionine gamma-synthase, eukaryotic translation initiation factor 5, geranylgeranyl pyrophosphate synthase, gibberellin 20-oxidase, glutamate-ammonia-ligase, glutaredoxin, glycosyl transferase, heat shock transcription factor, hydrolase, inositol-3-phosphate synthase, isochorismate synthase, leucyl/phenylalanyl-tRNA-protein transferase, major facilitator superfamily transporter protein, malate dehydrogenase, mevalonate kinase, mitochondrial inner membrane protease subunit, monodehydroascorbate reductase, multidrug resistance protein, NADH dehydrogenase subunit N, O-antigen chain length determinant, oxidoreductase, peroxisome assembly protein, potassium transport protein, protein kinase, purine nucleoside phosphorylase, rubredoxin, Sec-independent protein translocase subunit, serine protease, sll0064-protein, slr0383-protein, sodium/proton antiporter, thioredoxin family protein, threonine synthase, tryptophan synthase alpha chain, TTC0768-protein, TTC1386-protein, uroporphyrin-III C-methyltransferase, yer152c-protein, ylr065c-protein, yml093w-protein, and zinc finger protein are also called “FCRP genes”.

[0211.1.7.18] to [0225.1.7.18] for the disclosure of these paragraphs see [0211.1.7.7] to [0225.1.7.7] above

In addition to the sequence mentioned in Table I, application no. 18, column 5 or 8, preferably the coding region thereof, or its homologs or fragments, it may be advantageous additionally to express and/or mutate further genes in the non-human organisms. Especially advantageously, additionally at least one further gene of the biosynthetic pathway of the fine chemical is expressed in the non-human organisms such as plants or microorganisms. It is also possible that the regulation of the natural genes has been modified advantageously so that the gene and/or its gene product is no longer subject to the regulatory mechanisms which exist in the non-human organisms. This leads to an increased synthesis of the respective VLCFA since, for example, feedback regulations no longer exist to the same extent or not at all. In addition it might be advantageously to combine the nucleic acids sequences of the invention containing the sequences shown in the respective line in Table I, application no. 18, column 5 or 8, preferably the coding region thereof, or homologs or fragments thereof, with genes which generally support or enhance the growth or yield of the target non-human organisms, for example genes which lead to faster growth rate of non human organism like microorganisms or plants or genes which produce stress-, pathogen-, or herbicide-resistant plants.

In a further embodiment of the process of the invention, non-human organisms are grown, in which there is in addition to the expression of the nucleic acid molecule according to the invention simultaneous expression of at least one nucleic acid molecule or one of the genes which code for proteins involved in the fatty acid and/or waxes metabolism, in particular in VLCFA synthesis, especially genes selected from the group coding for polypeptides of the fatty acid pathway, such as acetyl-CoA or malonyl-CoA or a polypeptide having a very long chain fatty acid acyl (VLCFA) CoA synthase activity and/or genes selected from the group coding for Ketoacetyl-synthase (=VLCFA-synthase), e.g. FAE1 (AT4G34520) from A. thaliana, beta-Ketoacyl-CoA-reductase, beta-Hydroxyacyl-CoA-dehydratase, 2-trans-Enoyl-CoA-reductase and/or 3-ketoacyl-CoA synthases involved in VLCFA-biosynthesis and/or having the activity of a annotated gene selected from the group comprising AT1G04220 (KCS2), AT1G07720 (KCS3), AT1G19440 (KCS4), AT1G71160 (KCS7), AT2G15090 (KCS8), AT2G16280 (KCS9), AT2G26640 (KCSll), AT2G28630 (KCS12), AT2G46720 (KSC13), AT3G10280 (KCS14), AT3G52160 (KCS15), AT4G34250 (KCS16), AT4G34510 (KCS17), AT4G34520 (KCS18), AT5G04530 (KCS19), AT5G43760 (KCS20) and AT5G49070 (KCS21).

[0228.1.7.18] to [0239.1.7.18] for the disclosure of these paragraphs see [0228.1.7.7] to [0239.1.7.7] above

In an embodiment of the present invention the nucleic acid construct of the present invention comprises a nucleic acid molecule as depicted in the respective line in Table I, application no. 18, column 5 or 8, preferably the coding region thereof, a fragment or a homolog thereof, and a plant promoter, such as the Big 35S promoter, the DC3 promoter, the PC Ubi promoter, the LegB4-promoter, the Super promoter, the USP promoter or the like. In another embodiment the nucleic acid construct of the present invention comprises a nucleic acid molecule as depicted in the respective line in Table I, application no. 18, column 5 or 8, preferably the coding region thereof, a fragment or a homolog thereof, and a plant promoter such as, for example, the USP, the LegB4-, the DC3 promoter or the ubiquitin promoter from parsley or other herein mentioned promoters. In a further embodiment the nucleic acid construct of the present invention comprises a nucleic acid molecule as depicted in the respective line in Table I, application no. 18, column 5 or 8, preferably the coding region thereof, a fragment or a homolog thereof, and a plant promoter, such as the Big 35S promoter, the PC Ubi promoter, the Super promoter, the USP promoter or the like. In addition different terminators may advantageously be used in these nucleic acid constructs; examples for transcriptional termination are polyadenylation signals.

[0241.1.7.18] to [0245.1.7.18] for the disclosure of these paragraphs see [0241.1.7.7] to [0245.1.7.7] above

Other preferred sequences for use in operable linkage in gene expression constructs are targeting sequences, which are required for targeting the gene product into specific cell compartments (for a review, see Kermode, Crit. Rev. Plant Sci. 15 (4), 285 (1996) and references cited therein), for example into the vacuole, the nucleus, all types of plastids, such as amyloplasts, chloroplasts, chromoplasts, the extracellular space, the mitochondria, the endoplasmic reticulum, elaioplasts, peroxisomes, glycosomes, and other compartments of cells or extracellular; preferred are sequences, which are involved in targeting to plastids as mentioned above. Sequences, which must be mentioned in this context are, in particular, the signalpeptide-or transit-peptide-encoding sequences which are known per se. For example, plastid-transit-peptide-encoding sequences enable the targeting of the expression product into the plastids of a plant cell. Targeting sequences are also known for eukaryotic and to a lower extent for prokaryotic organisms and can advantageously be operable linked with the nucleic acid molecule of the present invention as shown in Table I, application no. 18, columns 5 and 8, preferably the coding region thereof, or homologs or fragments thereof, and described herein to achieve an expression in one of said compartments or extracellular.

[0247.1.7.18] to [0249.1.7.18] for the disclosure of these paragraphs see [0247.1.7.7] to [0249.1.7.7] above

Preferred promoters are in particular those which bring about gene expression in tissues and organs, such as leaves, roots, seeds, pollen or xylem. Examples of tissue preferred or organ preferred promoters include, but are not limited to fruit-preferred, ovule-preferred, male tissue-preferred, seed-preferred, pollen-preferred integument-preferred, tuber-preferred, stalk-preferred, pericarp-preferred, leaf-preferred, stigma-preferred, pollen-preferred, anther-preferred, petal-preferred, sepal-preferred, pedicel-preferred, silique-preferred, stemp-referred, root-preferred promoters, and the like. Especially those promoters are preferred which bring about gene expression in tissues and organs in which the biosynthesis of waxes and/or VLCFAs takes place, like in seed cells, such as endosperm cells and cells of the developing embryo. Seed promoters are preferentially expressed during seed development and/or germination. For example, seed preferred promoters can be embryo-preferred, endosperm preferred and seed coat-preferred (see Thompson et al., BioEssays 10, 108 (1989)). Examples of seed preferred promoters include, but are not limited to, cellulose synthase (celA), Cim1, gamma-zein, globulin-1, maize 19 kD zein (cZ19B1), and the like. Other suitable promoters are the oilseed rape napin gene promoter (U.S. Pat. No. 5,608,152), the Vicia faba USP promoter (Baeumlein et al., Mol Gen Genet, 225 (3), 459 (1991)), the Arabidopsis oleosin promoter (WO 98/45461), the Phaseolus vulgaris phaseolin promoter (U.S. Pat. No. 5,504,200), the Brassica Bce4 promoter (WO 91/13980), the bean arcs promoter, the carrot DcG3 promoter, or the Legumin B4 promoter (LeB4) (Baeumlein et al., Plant Journal, 2 (2), 233 (1992)), and promoters which bring about the seed-specific expression in monocotyledonous plants such as maize, barley, wheat, rye, rice and the like. Advantageous seed-specific promoters are the sucrose binding protein promoter (WO 00/26388), the phaseolin promoter and the napin promoter. Suitable promoters which must be considered are the barley Ipt2 or Ipt1 gene promoter (WO 95/15389 and WO 95/23230), and the promoters described in WO 99/16890 (promoters from the barley hordein gene, the rice glutelin gene, the rice oryzin gene, the rice prolamin gene, the wheat gliadin gene, the wheat glutelin gene, the maize zein gene, the oat glutelin gene, the sorghum kasirin gene and the rye secalin gene). Further suitable promoters are Amy32b, Amy 6-6 and Aleurain (U.S. Pat. No. 5,677,474), Bce4 (oilseed rape) (U.S. Pat. No. 5,530,149), glycinin (soya) (EP 571 741], phosphoenolpyruvate carboxylase (soya) (JP 06/62870), ADR12-2 (soya) (WO 98/08962), isocitrate lyase (oilseed rape) (U.S. Pat. No. 5,689,040]) or α-amylase (barley) (EP 781 8499. Other promoters which are available for the expression of genes in plants are leaf-specific promoters such as those described in DE-A 19 644 478 or light-regulated promoters such as, for example, the pea petE promoter.

[0251.1.7.18] to [0266.1.7.18] for the disclosure of these paragraphs see [0251.1.7.7] to [0266.1.7.7] above

An embodiment of the invention relates to a vector where the nucleic acid molecule according to the invention is linked operably to regulatory sequences which permit the expression in a prokaryotic or eukaryotic host. A further embodiment of the invention relates to a vector in which a nucleic acid sequence encoding one of the polypeptides shown in the respective line in Table II, application no. 18, columns 5 or 8, or homologs or fragments thereof, is functionally linked to a targeting sequence such as a plastidial or mitochondrial targeting sequence. A further embodiment of the invention relates to a vector in which a nucleic acid sequence encoding one of the polypeptides shown in the respective line in Table II, application no. 18, columns 5 or 8, or homologs or fragments thereof, is functionally linked to (a) regulatory sequence which permit the expression in an organelle, such as plastids or mitochondria, preferably plastids.

A further embodiment of the invention relates to a vector which comprises a nucleic acid sequence encoding one of the polypeptides shown in the respective line in Table II, application no. 18, columns 5 or 8, or homologs or fragments thereof, but no additional targeting sequence.

[0268.1.7.18] to [0273.1.7.18] for the disclosure of these paragraphs see [0268.1.7.7] to [0273.1.7.7] above

A further object of the invention relates to the use of a nucleic acid construct, e.g. an expression cassette, comprising one or more nucleic acid molecules encoding one or more polypeptides as depicted in the respective line(s) in Table II, application no. 18, columns 5 or 8, or homologs or fragments thereof, or comprising one or more nucleic acid molecules as depicted in the respective line(s) in Table I, application no. 18, columns 5 or 8, preferably the coding region thereof, or homologs or fragments thereof, or comprising a nucleic acid molecule hybridizing therewith for the transformation of a microorganism or a plant cell, plant tissues or a part of a plant

In doing so, depending on the choice of promoter, the nucleic acid molecules shown in the respective line in Table I, application no. 18, column 5 or 8, preferably the coding region thereof, or homologs or fragments thereof, or the nucleic acid molecules encoding a polypeptide as depicted in the respective line in Table II, application no. 18, column 5 or 8, or homologs or fragments thereof, can be expressed in the whole plant or specifically e.g. in the leaves, in the seeds, the nodules, in roots, in the stem or other parts of the plant. Those transgenic plants overproducing sequences, e.g. as depicted in the respective line in Table I, application no. 18, column 5 or 8, preferably the coding region thereof, or homologs or fragments thereof, the plant cell, plant tissues, plants, the reproductive material thereof, or parts thereof are a further object of the present invention.

Within the framework of the present invention, the production of the cerotic acid, heneicosanoic acid, lignoceric acid, melissic acid or montanic acid is due to the generation or over-expression of one or more polypeptides as depicted in the respective line(s) in Table II, application no. 18, column 5 or 8, or homologs or fragments thereof, or encoded by the corresponding nucleic acid molecules as depicted in the respective line(s) in Table I, application no. 18, column 5 or 8, preferably the coding region thereof, or homologs or fragments thereof, in the non-human organism according to the invention, advantageously in the transgenic microorganism or plant according to the invention, at least for the duration of at least one plant generation.

A constitutive expression of the polypeptide as depicted in the respective line in Table II, application no. 18, column 5 or 8, or homologs or fragments thereof, or encoded by the respective nucleic acid molecule as depicted in the respective line in Table I, application no. 18, column 5 or 8, preferably the coding region thereof, or homologs or fragments thereof, may be advantageous. On the other hand, an inducible expression may also appear desirable. Expression of the polypeptide sequences of the invention can be either directed to the cytosol or to the organelles, such as plastids or mitochondria, preferably the plastids of the host cells, preferably the plant cells, or non-targeted.

for the disclosure of this paragraph see [0278.1.7.7] above

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 18, column 5 or 8, preferably shown in Table II A, application no. 18, column 5, or in Table II A, application no. 18, column 8, or in Table II B, application no. 18, column 8, or a homolog or a fragment thereof;
   (b) a nucleic acid molecule shown in Table I, application no. 18, column 5 or 8, preferably shown in Table I A, application no. 18, column 5, or in Table I A, application no. 18, column 8, or in Table I B, application no. 18, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 18, preferably shown in Table II A, application no. 18, column 5, or in Table II A, application no. 18, column 8, or in Table II B, application no. 18, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 18, preferably shown in Table I A, application no. 18, column 5, or in Table I A, application no. 18, column 8, or in Table I B, application no. 18, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 18;
   (i) a nucleic acid molecule encoding a polypeptide having the activity represented by a respective protein as depicted in column 5 of Table II, application no. 18;
   (j) a nucleic acid molecule comprising a polynucleotide which is obtained by amplifying a cDNA library or a genomic library using the primers in Table III, application no. 18, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising an expression cassette comprising
[0) a promoter, preferably a promoter selected from the group consisting of Big35S, PCUbi, Super and USP; and 1) a nucleic acid molecule selected from the group consisting of
   (a) a nucleic acid molecule encoding a polypeptide shown in Table II, application no. 18, column 5 or 8, preferably shown in Table II A, application no. 18, column 5, or in Table II A, application no. 18, column 8, or in Table II B, application no. 18, column 8, or a homolog or a fragment thereof;
   (b) a nucleic acid molecule shown in Table I, application no. 18, column 5 or 8, preferably shown in Table I A, application no. 18, column 5, or in Table I A, application no. 18, column 8, or in Table I B, application no. 18, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 18, preferably shown in Table II A, application no. 18, column 5, or in Table II A, application no. 18, column 8, or in Table II B, application no. 18, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 18, preferably shown in Table I A, application no. 18, column 5, or in Table I A, application no. 18, column 8, or in Table I B, application no. 18, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 18;
   (i) a nucleic acid molecule encoding a polypeptide having the activity represented by a respective protein as depicted in column 5 of Table II, application no. 18;
   (j) a nucleic acid molecule comprising a polynucleotide which is obtained by amplifying a cDNA library or a genomic library using the primers in Table III, application no. 18, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising or being transformed by a vector comprising
[1) a nucleic acid molecule selected from the group consisting of
   (a) a nucleic acid molecule encoding a polypeptide shown in Table II, application no. 18, column 5 or 8, preferably shown in Table II A, application no. 18, column 5, or in Table II A, application no. 18, column 8, or in Table II B, application no. 18, column 8, or a homolog or a fragment thereof;
   (b) a nucleic acid molecule shown in Table I, application no. 18, column 5 or 8, preferably shown in Table I A, application no. 18, column 5, or in Table I A, application no. 18, column 8, or in Table I B, application no. 18, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 18, preferably shown in Table II A, application no. 18, column 5, or in Table II A, application no. 18, column 8, or in Table II B, application no. 18, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 18, preferably shown in Table I A, application no. 18, column 5, or in Table I A, application no. 18, column 8, or in Table I B, application no. 18, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 18;
   (i) a nucleic acid molecule encoding a polypeptide having the activity represented by a respective protein as depicted in column 5 of Table II, application no. 18;
   (j) a nucleic acid molecule comprising a polynucleotide which is obtained by amplifying a cDNA library or a genomic library using the primers in Table III, application no. 18, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 18.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 18 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 18, in the same line as of the respective nucleic acid molecule according to 1) “plastidic” is depicted, or encoding a mitochondrial transit peptide, especially in case in column 6 of Table I, application no. 18, in the same line as of the respective nucleic acid molecule according to 1) “mitochondric” is depicted.

In another embodiment of said transgenic non-human organism the expression cassette does not comprise 2) a nucleic acid molecule encoding a transit peptide, especially in case in column 6 of Table I, application no. 18, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 18.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 18, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 18, in the same line as of the respective nucleic acid molecule according to 1) “plastidic” is depicted, or encoding a mitochondrial transit peptide, especially in case in column 6 of Table I, application no. 18, in the same line as of the respective nucleic acid molecule according to 1) “mitochondric” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 18, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 18, in the same line as of the respective nucleic acid molecule according to 1) “plastidic” is depicted, or encoding a mitochondrial transit peptide, especially in case in column 6 of Table I, application no. 18, in the same line as of the respective nucleic acid molecule according to 1) “mitochondric” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 18, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 18, in the same line as of the respective nucleic acid molecule according to 1) “plastidic” is depicted, or encoding a mitochondrial transit peptide, especially in case in column 6 of Table I, application no. 18, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 18.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 18, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 18.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 18, in the same line as of the respective nucleic acid molecule according to 1) “plastidic” is depicted, or encoding a mitochondrial transit peptide, especially in case in column 6 of Table I, application no. 18, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 18, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 18, in the same line as of the respective nucleic acid molecule according to 1) “plastidic” is depicted, or encoding a mitochondrial transit peptide, especially in case in column 6 of Table I, application no. 18, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 18 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 18, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 18 (in the same line) and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

In a further embodiment the invention relates to a transgenic non-human organisms such as transgenic microorganism or transgenic plant comprising a nucleic acid molecule comprising
[1) a nucleic acid molecule selected from the group consisting of
   (a) a nucleic acid molecule encoding a polypeptide shown in Table II, application no. 18, column 5 or 8, preferably shown in Table II A, application no. 18, column 5, or in Table II A, application no. 18, column 8, or in Table II B, application no. 18, column 8, or a homolog or a fragment thereof;
   (b) a nucleic acid molecule shown in Table I, application no. 18, column 5 or 8, preferably shown in Table I A, application no. 18, column 5, or in Table I A, application no. 18, column 8, or in Table I B, application no. 18, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 18, preferably shown in Table II A, application no. 18, column 5, or in Table II A, application no. 18, column 8, or in Table II B, application no. 18, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 18, preferably shown in Table I A, application no. 18, column 5, or in Table I A, application no. 18, column 8, or in Table I B, application no. 18, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 18;
   (i) a nucleic acid molecule encoding a polypeptide having the activity represented by a respective protein as depicted in column 5 of Table II, application no. 18;
   (j) a nucleic acid molecule comprising a polynucleotide which is obtained by amplifying a cDNA library or a genomic library using the primers in Table III, application no. 18, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;
      and, if desired
2) nucleic acid molecule encoding a transit peptide, preferably encoding a plastidal transit peptide or a mitochondrial transit peptide;
   which are operable linked;]
   or comprising an expression cassette comprising
[0) a promoter, preferably a promoter selected from the group consisting of Big35S, PCUbi, Super and USP; and
1) a nucleic acid molecule selected from the group consisting of
   (a) a nucleic acid molecule encoding a polypeptide shown in Table II, application no. 18, column 5 or 8, preferably shown in Table II A, application no. 18, column 5, or in Table II A, application no. 18, column 8, or in Table II B, application no. 18, column 8, or a homolog or a fragment thereof;
   (b) a nucleic acid molecule shown in Table I, application no. 18, column 5 or 8, preferably shown in Table I A, application no. 18, column 5, or in Table I A, application no. 18, column 8, or in Table I B, application no. 18, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 18, preferably shown in Table II A, application no. 18, column 5, or in Table II A, application no. 18, column 8, or in Table II B, application no. 18, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 18, preferably shown in Table I A, application no. 18, column 5, or in Table I A, application no. 18, column 8, or in Table I B, application no. 18, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 18;
   (i) a nucleic acid molecule encoding a polypeptide having the activity represented by a respective protein as depicted in column 5 of Table II, application no. 18;
   (j) a nucleic acid molecule comprising a polynucleotide which is obtained by amplifying a cDNA library or a genomic library using the primers in Table III, application no. 18, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      and, if desired
2) nucleic acid molecule encoding a transit peptide, preferably encoding a plastidal transit peptide or a mitochondrial transit peptide; which are operable linked;]
   or comprising or being transformed by a vector comprising
[1) a nucleic acid molecule selected from the group consisting of
   (a) a nucleic acid molecule encoding a polypeptide shown in Table II, application no. 18, column 5 or 8, preferably shown in Table II A, application no. 18, column 5, or in Table II A, application no. 18, column 8, or in Table II B, application no. 18, column 8, or a homolog or a fragment thereof;
   (b) a nucleic acid molecule shown in Table I, application no. 18, column 5 or 8, preferably shown in Table I A, application no. 18, column 5, or in Table I A, application no. 18, column 8, or in Table I B, application no. 18, column 8, preferably the coding region thereof, or a homolog or a fragment thereof;
   (c) a nucleic acid molecule, which, as a result of the degeneracy of the genetic code, can be derived from a polypeptide sequence depicted in column 5 or 8 of Table II, application no. 18, preferably shown in Table II A, application no. 18, column 5, or in Table II A, application no. 18, column 8, or in Table II B, application no. 18, column 8;
   (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 18, preferably shown in Table I A, application no. 18, column 5, or in Table I A, application no. 18, column 8, or in Table I B, application no. 18, column 8, preferably the coding region thereof;
   (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no. 18;
   (i) a nucleic acid molecule encoding a polypeptide having the activity represented by a respective protein as depicted in column 5 of Table II, application no. 18;
   (j) a nucleic acid molecule comprising a polynucleotide which is obtained by amplifying a cDNA library or a genomic library using the primers in Table III, application no. 18, column 8;
   and
   (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   or a nucleic acid molecule comprising a sequence which is complementary thereto;]
      whereby, preferably, the nucleic acid molecule according to 1 (a), (b), (c), (d), (e), (f), (g), (h), (i), (j) or (k) distinguishes over the sequence depicted in Table I A and/or I B, application no. 18, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 18, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 18, column 5 or 8, or the coding region thereof, respectively. In a further embodiment the nucleic acid molecule does not encode the polypeptide sequence shown in Table II A and/or II B, application no. 18, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 18, column 5 or 8 and does not encode a protein of the sequence shown in Table II A and/or II B, application no. 18, column 5 or 8. Accordingly, in one embodiment, the protein encoded by a sequence of a nucleic acid according to 1 (a), (b), (c), (d), (e), (f), (g), (h), (i), (j) or (k) does not consist of the sequence shown in Table I A and/or I B, application no. 18, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 18, column 5 or 8 but less than 100%, preferably less than 99.999%, 99.99%, 99.9%, 99%, 98%, 97%, 96% or 95% identical to the sequence shown in Table II A and/or II B, application no. 18, column 5 or 8; as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 18.

In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 18 and confers a generation or an increase of the respective fine chemical in a non-human organism or a part thereof as compared to a corresponding wild-type non-human organism or part thereof.

In another embodiment of said transgenic non-human organism the expression cassette comprises 2) a nucleic acid molecule encoding a transit peptide, preferably encoding a plastidal transit peptide, especially in case in column 6 of Table I, application no. 18, in the same line as of the respective nucleic acid molecule according to 1) “plastidic” is depicted, or encoding a mitochondrial transit peptide, especially in case in column 6 of Table I, application no. 18, in the same line as of the respective nucleic acid molecule according to 1) “mitochondric” is depicted.

In another embodiment thereof the expression cassette does not comprise 2) a nucleic acid molecule encoding a transit peptide, especially in case in column 6 of Table I, application no. 18, in the same line as of the respective nucleic acid molecule according to 1) “non-targeted” is depicted.

In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 18.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the same line in column 5 of Table II, application no. 18, and confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 18, in the same line as of the respective nucleic acid molecule according to 1) “plastidic” is depicted, or encoding a mitochondrial transit peptide, especially in case in column 6 of Table I, application no. 18, in the same line as of the respective nucleic acid molecule according to 1) “mitochondric” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 18, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 18, in the same line as of the respective nucleic acid molecule according to 1) “plastidic” is depicted, or encoding a mitochondrial transit peptide, especially in case in column 6 of Table I, application no. 18, in the same line as of the respective nucleic acid molecule according to 1) “mitochondric” is depicted, which are operable linked.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 18, in the same line as of the respective nucleic acid molecule according to 1) “non-targeted” is depicted, which are operable linked.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 18, in the same line as of the respective nucleic acid molecule according to 1) “plastidic” is depicted, or encoding a mitochondrial transit peptide, especially in case in column 6 of Table I, application no. 18, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 18.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 18, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 18.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 18, in the same line as of the respective nucleic acid molecule according to 1) “plastidic” is depicted, or encoding a mitochondrial transit peptide, especially in case in column 6 of Table I, application no. 18, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 18, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 18, in the same line as of the respective nucleic acid molecule according to 1) “plastidic” is depicted, or encoding a mitochondrial transit peptide, especially in case in column 6 of Table I, application no. 18, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 18 and confers a generation or an increase of the respective fine chemical in a non-human organism or a part thereof as compared to a corresponding wild-type non-human organism or part thereof.

In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 18, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 18 and confers a generation or an increase of the respective fine chemical in a non-human organism or a part thereof as compared to a corresponding wild-type non-human organism or part thereof.

In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

[0291.1.7.18] to [0299.1.7.18] for the disclosure of these paragraphs see [0291.1.7.7] to [0299.1.7.7] above

The term “biological active portion” or “biological activity” means a polypeptide as depicted in the respective line in Table II, application no. 18, column 3 or a portion of said polypeptide which still has at least 10% or 20%, preferably 30%, 40%, 50% or 60%, especially preferably 70%, 75%, 80%, 90% or 95% of the enzymatic or biological activity of the natural or starting enzyme or protein.

[0301.1.7.18] to [0304.1.7.18] for the disclosure of these paragraphs see [0301.1.7.7] to [0304.1.7.7] above

Synthetic oligonucleotide primers for the amplification, e.g. as shown in the respective line in Table III, application no. 18, column 8, by means of polymerase chain reaction can be generated on the basis of a sequence shown herein, for example the sequence shown in the respective line in Table I, application no. 18, columns 5 or 8, or the sequences derived from Table II, application no. 18, columns 5 or 8, respectively.

Moreover, it is possible to identify a conserved region of a protein from various organism by carrying out protein sequence alignments with the polypeptide encoded by the nucleic acid molecules of the present invention, in particular with the sequences encoded by the nucleic acid molecule shown in the respective line in column 5 or 8 of Table I, application no. 18, from which conserved regions, and in turn, degenerate primers can be derived. Conserved regions are those, which show a very little variation in the amino acid in one particular position of several homologs from different origin. The consensus sequence and polypeptide motifs shown in the respective line in column 8 of Table IV, application no. 18, are derived from such alignments of homologous proteins.

In an embodiment of the present invention, in the method of the present invention the activity of a polypeptide comprising or consisting of a consensus sequence or at least one polypeptide motif shown in the respective line in Table IV, application no. 18, column 8, is increased. In another embodiment thereof, the method of the present invention relates to a polypeptide comprising or consisting of a consensus sequence shown in the respective line in Table IV, application no. 18, column 8 or comprising at least one polypeptide motif shown in the respective line in Table IV, application no. 18, column 8, whereby not more than 20, 15, 10, 9, 8, 7, 6, 5 4, 3, 2 or 1, or 0 of the amino acid positions indicated can be replaced by any amino acid. In another embodiment thereof, the method of the present invention relates to a polypeptide comprising or consisting of a consensus sequence shown in the respective line in Table IV, application no. 18, column 8 or comprising at least one polypeptide motif shown in the respective line in Table IV, application no. 18, column 8, whereby not more than 15%, 10%, 5%, 4%, 3%, 2% or 1%, or 0% of the amino acid positions defined as distinct specific amino acids are/is replaced by another amino acid. In another embodiment thereof, the method of the present invention relates to a polypeptide comprising or consisting of a consensus sequence shown in the respective line in Table IV, application no. 18, column 8 or comprising at least one polypeptide motif shown in the respective line in Table IV, application no. 18, column 8, whereby not more than 20, 15, 10, 9, 8, 7, 6, 5, 4, 3, 2 or 1, or 0 amino acids are inserted into a consensus sequence or polypeptide motif.

In an embodiment of the present invention, in the method of the present invention the activity of a polypeptide comprising or consisting of a consensus sequence or at least one polypeptide motif shown in the respective line in Table IV, application no. 18, column 8, is increased, whereby said polypeptide distinguishes over the sequence depicted in Table II, application no. 18, columns 5 or 8 by one or more amino acids.

In one embodiment, said polypeptide of the invention distinguishes over the sequence shown in the respective line in Table II, application no. 18, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II B, application no. 18, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in Table II, application no. 18, columns 5 or 8.

[0309.1.7.18] to [0321.1.7.18] for the disclosure of these paragraphs see [0309.1.7.7] to [0321.1.7.7] above

Polypeptides having above-mentioned activity, i.e. conferring the production or the increased production of the fine chemical cerotic acid, heneicosanoic acid, lignoceric acid, melissic acid or montanic acid as compared to a corresponding, e.g. non-transformed, wild type non-human organism, like a microorganism or a plant cell, plant or part thereof, can be encoded by other DNA sequences which hybridize to the sequences shown in the respective line in Table I, application no. 18, columns 5 and 8, preferably the coding region thereof, at least under relaxed hybridization conditions and which encode the expression of polypeptides conferring the production or the increased production of the respective fine chemical cerotic acid, heneicosanoic acid, lignoceric acid, melissic acid or montanic acid as compared to a corresponding, e.g. non-transformed, wild type non-human organism, like a microorganism or a plant cell, plant or part thereof.

[0323.1.7.18] to [0329.1.7.18] for the disclosure of these paragraphs see [0323.1.7.7] to [0329.1.7.7] above

Further, the nucleic acid molecule of the invention comprises a nucleic acid molecule, which is a complement of one of the nucleotide sequences of above-mentioned nucleic acid molecules or a portion thereof. A nucleic acid molecule or its sequence which is complementary to one of the nucleotide molecules or sequences shown in the respective line in Table I, application no. 18, columns 5 or 8, preferably the coding region thereof, homologs or fragments thereof, is one which is sufficiently complementary to one of the nucleotide molecules or sequences shown in the respective line in Table I, application no. 18, columns 5 or 8, preferably the coding region thereof, homologs or fragments thereof, such that it can hybridize to one of the nucleotide sequences shown in Table I, application no. 18, columns 5 or 8, preferably the coding region thereof, homologs or fragments thereof, thereby forming a stable duplex. Preferably, the hybridization is performed under stringent hybridization conditions. However, a complement of one of the herein disclosed sequences is preferably a sequence complement thereto according to the base pairing of nucleic acid molecules well known to the skilled person. For example, the bases A and G undergo base pairing with the bases T and U or C, resp. and visa versa. Modifications of the bases can influence the base-pairing partner.

The nucleic acid molecule of the invention comprises a nucleotide sequence which is at least about 30%, 35%, 40%, 45%, 50%, 55%, 60%, 65%, 70%, 75%, 80%, 90%, 95%, 96%, 97%, 98%, 99%, 99.5% or more homologous to a nucleotide sequence shown in Table I, application no. 18, columns 5 or 8, preferably the coding region thereof, homologs or fragments thereof, and preferably has above-mentioned activity, in particular conferring the production or the increased production of the fine chemical cerotic acid, heneicosanoic acid, lignoceric acid, melissic acid or montanic acid, respectively, after increasing the activity or an activity of a gene as shown in the respective line in Table I or of a gene product, e.g. as shown in the respective line in Table II, application no. 18, column 5 or 8, by for example in one embodiment expression either in the cytosol or in an organelle such as a plastid or mitochondria or both, preferably in a plastid, or in another embodiment non-targeted or targeted.

The nucleic acid molecule of the invention comprises a nucleotide sequence or molecule which hybridizes, preferably hybridizes under stringent conditions as defined herein, to one of the nucleic acid molecule shown in the respective line in Table I, preferably Table IB, application no. 18, columns 5 or 8, preferably the coding region thereof, fragments or homologs thereof, and encodes a protein having above-mentioned activity, e.g. conferring the production or the increased production of the respective fine chemical cerotic acid, heneicosanoic acid, lignoceric acid, melissic acid or montanic acid as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted, and optionally, the activity thereof is selected from the group consisting of 2,3-dihydroxy-2,3-dihydrophenylpropionatedehydrogenase, ABC metal ion transporter substrate-binding protein, acetyl-coenzyme A synthetase, acetyltransferase, acid shock protein, acyl transferase, agmatinase, aminotransferase, ATP-binding component of a transport system, b1522-protein, b3083-protein, b3644-protein, b3812-protein, b4029-protein, binding-protein-dependent transport systems inner membrane component, calcium-dependent protein kinase, cardiolipin synthase, chaperone protein CIpB, cyclin D, cystathionine gamma-synthase, eukaryotic translation initiation factor 5, geranylgeranyl pyrophosphate synthase, gibberellin 20-oxidase, glutamate-ammonia-ligase, glutaredoxin, glycosyl transferase, heat shock transcription factor, hydrolase, inositol-3-phosphate synthase, isochorismate synthase, leucyl/phenylalanyl-tRNA-protein transferase, major facilitator superfamily transporter protein, malate dehydrogenase, mevalonate kinase, mitochondrial inner membrane protease subunit, monodehydroascorbate reductase, multidrug resistance protein, NADH dehydrogenase subunit N, O-antigen chain length determinant, oxidoreductase, peroxisome assembly protein, potassium transport protein, protein kinase, purine nucleoside phosphorylase, rubredoxin, Sec-independent protein translocase subunit, serine protease, sll0064-protein, slr0383-protein, sodium/proton antiporter, thioredoxin family protein, threonine synthase, tryptophan synthase alpha chain, TTC0768-protein, TTC1386-protein, uroporphyrin-III C-methyltransferase, yer152c-protein, ylr065c-protein, yml093w-protein, and zinc finger protein, respectively.

Moreover, the nucleic acid molecule of the invention can comprise only a portion of the coding region of one of the nucleic acid molecules depicted in Table I, application no. 18, columns 5 or 8, for example a fragment which can be used as a probe or primer or a fragment encoding a biologically active portion of the polypeptide of the present invention or of a polypeptide used in the process of the present invention, i.e. having above-mentioned activity, e.g. conferring the production or the increased production of the respective fine chemical cerotic acid, heneicosanoic acid, lignoceric acid, melissic acid or montanic acid as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted. The nucleotide sequences determined from the cloning of the present protein-according-to-the-invention-encoding gene allows for the generation of probes and primers designed for use in identifying and/or cloning its homologues from other cell types and organisms. The probe/primer typically comprises a substantially purified oligonucleotide. The oligonucleotide typically comprises a region of nucleotide sequence that hybridizes under stringent conditions to at least about 12, 15 preferably about 20 or 25, more preferably about 40, 50 or 75 consecutive nucleotides of a sense strand of one of the sequences set forth, e.g., in the respective line in Table I, application no. 18, column 5 or 8, preferably the coding region thereof, or a homolog or a fragment thereof, an anti-sense sequence of one of the sequences, e.g., set forth in the respective line in Table I, application no. 18, columns 5 or 8, preferably the coding region thereof, or a homolog or a fragment thereof or naturally occurring mutants thereof. Primers based on a nucleic acid molecule of invention can be used in PCR reactions to clone homologs of the polypeptide of the invention or of the polypeptide used in the process of the invention, e.g. as the primers described in the examples of the present invention, e.g. as shown in the examples. A PCR with the primers shown in the respective line in Table Ill, column 8 will result in a fragment of the gene product as shown in Table II, application no. 18, column 5.

Primer sets are interchangeable. The person skilled in the art knows how to design and combine said primers to result in the desired product, e.g. in a full length clone or a partial sequence. Probes based on the sequences of the nucleic acid molecule of the invention or used in the process of the present invention can be used to detect transcripts or genomic sequences encoding the same or homologous proteins. The probe can further comprise a label group attached thereto, e.g. the label group can be a radioisotope, a fluorescent compound, an enzyme, or an enzyme co-factor. Such probes can be used as a part of a genomic marker test kit for identifying cells which express a polypeptide of the invention or used in the process of the present invention, such as by measuring a level of an encoding nucleic acid molecule in a sample of cells, e.g. detecting mRNA levels or determining, whether a genomic gene comprising the sequence of the polynucleotide of the invention or used in the processes of the present invention has been mutated or deleted.

As the expression of a respective nucleic acids of the invention is related to the synthesis of the respective fine chemical cerotic acid, heneicosanoic acid, lignoceric acid, melissic acid or montanic acid its function as a probe extends to the detection of microorganisms, plant tissues, plants, plant variets, plant ecotypes or plant genera with varying capability or potential for synthesis of the respective fine chemical cerotic acid, heneicosanoic acid, lignoceric acid, melissic acid or montanic acid. Therefore in one embodiment the present invention relates to a method for analyzing the capability or potential of a plant tissue, a plant, a plant variety or plant ecotype to produce the fine chemical cerotic acid, heneicosanoic acid, lignoceric acid, melissic acid or montanic acid by using the nucleic acid of the invention or parts thereof as a probe to detect the amount of the nucleic acid of the invention in the non-human organism or a part thereof in comparison to another non-human organism.

The nucleic acid molecule of the invention encodes a polypeptide or portion thereof which includes an amino acid sequence which is sufficiently homologous to the amino acid sequence shown in respective line in Table II, application no. 18, columns 5 or 8 such that the protein or portion thereof maintains the ability to confer the production or the increased production of the respective fine chemical cerotic acid, heneicosanoic acid, lignoceric acid, melissic acid or montanic acid as compared to a corresponding, e.g. non-transformed, wild type non-human organism, like a microorganism or a plant cell, plant or part thereof, in particular increasing the activity as mentioned above or as described in the examples in microorganisms or plants.

As used herein, the language “sufficiently homologous” refers to proteins or portions thereof which have amino acid sequences which include a minimum number of identical or equivalent amino acids (e.g. an amino acid which has a similar side chain as an amino acid in one of the sequences of the polypeptide of the present invention) to an amino acid sequence shown in the respective line in Table II, application no. 18, columns 5 or 8 such that the protein or portion thereof is able to confer the production or the increased production of the respective fine chemical cerotic acid, heneicosanoic acid, lignoceric acid, melissic acid or montanic acid as compared to a corresponding, e.g. non-transformed, wild type non-human organism, like a microorganism or a plant cell, plant or part thereof. For example having the activity of a protein as shown in the respective line in Table II, application no. 18, column 3 and as described herein.

In an embodiment of the present invention, the nucleic acid molecule of the present invention comprises a nucleic acid molecule that encodes a portion of the protein of the present invention. The protein is at least about 30%, 35%, 40%, 45% 50%, 55%, 60%, 65%, 70%, 75%, 80%, 85%, 90%, 91%, 92%, 93%, 94%, 95%, 96%, 97%, 98%, 99%, 99.5% or more homologous to an entire amino acid sequence shown in the respective line of Table II, application no. 18, column 5 or 8 and has the above-mentioned activity, e.g. conferring the production or the increased production of the respective fine chemical cerotic acid, heneicosanoic acid, lignoceric acid, melissic acid or montanic acid as compared to a corresponding, e.g. non-transformed, wild type non-human organism, like a microorganism or a plant cell, plant or part thereof by, for example in a embodiment expression either in the cytosol or in an organelle such as a plastid or mitochondria or both, preferably in a plastid, or in another embodiment by targeted or non-targeted expression.

for the disclosure of this paragraph see [0338.1.7.7] above

for the disclosure of this paragraph see [0339.1.7.7] above

The invention further relates to nucleic acid molecules or methods using said nucleic acid molecules that differ from one of the nucleotide sequences shown in the respective line in Table I, application no. 18, columns 5 or 8, preferably the coding region thereof, or fragments or homologs thereof, due to degeneracy of the genetic code but encode a polypeptide of the present invention, in particular a polypeptide having the above-mentioned activity, e.g. as those polypeptides depicted in the respective line in Table II, application no. 18, columns 5 or 8, fragments or homologs thereof. Advantageously, the nucleic acid molecule of the invention comprises, or in an other embodiment has, a nucleotide sequence encoding a protein comprising, or in an other embodiment having, an amino acid sequence shown in the respective line in Table II, application no. 18, columns 5 or 8, fragments or homologs thereof. In a further embodiment, the nucleic acid molecule of the invention encodes a full length protein which is substantially homologous to an amino acid sequence shown in the respective line in Table II, application no. 18, columns 5 or 8, or homologs thereof. However, in an embodiment, the nucleic acid molecule of the present invention does not consist of the sequence shown in the respective line in Table I, preferably Table IA, application no. 18, columns 5 or 8, preferably the coding region thereof, but the functional homologs thereof.

[0341.1.7.18] to [0343.1.7.18] for the disclosure of these paragraphs see [0341.1.7.7] to [0343.1.7.7] above

Accordingly, in another embodiment, a nucleic acid molecule of the invention is at least 15, 20, 25 or 30 nucleotides in length. Preferably, it hybridizes under stringent conditions to a nucleic acid molecule comprising a nucleotide sequence of the nucleic acid molecule of the present invention or used in the process of the present invention, e.g. comprising the sequence shown in the respective line in Table I, application no. 18, columns 5 or 8, preferably the coding region thereof. In another embodiment the nucleic acid molecule is preferably at least 20, 30, 50, 100, 250 or more nucleotides in length.

for the disclosure of this paragraph see [0345.1.7.7] above

Preferably, a nucleic acid molecule of the invention that hybridizes under stringent conditions to a sequence shown in the respective line in Table I, application no. 18, columns 5 or 8, preferably the coding region thereof, corresponds to a naturally-occurring nucleic acid molecule of the invention. As used herein, a “naturally-occurring” nucleic acid molecule refers to a RNA or DNA molecule having a nucleotide sequence that occurs in nature (e.g. encodes a natural protein). Preferably, the nucleic acid molecule encodes a natural protein having above-mentioned activity, e.g. conferring the production or the increased production of the respective fine chemical cerotic acid, heneicosanoic acid, lignoceric acid, melissic acid or montanic acid as compared to a corresponding, e.g. non-transformed, wild type non-human organism, like a microorganism or a plant cell, plant or part thereof after increasing the expression or activity thereof or the activity of a protein of the invention or used in the process of the invention, in an embodiment for example expression either in the cytosol or in an organelle such as a plastid or mitochondria, preferably in plastids, or, in another embodiment by targeted or non-targeted expression.

for the disclosure of this paragraph see [0347.1.7.7] above

For example, nucleotide substitutions leading to amino acid substitutions at “non-essential” amino acid residues can be made in a sequence of the nucleic acid molecule of the invention or used in the process of the invention, e.g. shown in the respective line in Table I, application no. 18, columns 5 or 8, preferably the coding region thereof, or fragments or homologs thereof.

for the disclosure of this paragraph see [0349.1.7.7] above

for the disclosure of this paragraph see [0350.1.7.7] above

Accordingly, the invention relates to nucleic acid molecules encoding a polypeptide having above-mentioned activity, in a non-human organisms or parts thereof by for example expression either in the cytosol or in an organelle such as a plastid or mitochondria or both, preferably in plastids, that contain changes in amino acid residues that are not essential for said activity. Such polypeptides differ in amino acid sequence from a sequence depicted in the respective line in Table II, especially Table IIA, application no. 18, columns 5 or 8, but retain said activity described herein. The nucleic acid molecule can comprise a nucleotide sequence encoding a polypeptide, wherein the polypeptide comprises an amino acid sequence at least about 50% identical to an amino acid sequence shown in the respective line in Table II, application no. 18, columns 5 or 8, and is capable of conferring the production or the increased production of the respective fine chemical cerotic acid, heneicosanoic acid, lignoceric acid, melissic acid or montanic acid as compared to a corresponding, e.g. non-transformed, wild type non-human organism, like a microorganism or a plant cell, plant or part thereof, after increasing its activity for example in an embodiment by expression either in the cytosol or in an organelle such as a plastid or mitochondria or both, preferably in plastids, or, in another embodiment by targeted or non-targeted expression. Preferably, the protein encoded by the nucleic acid molecule is at least about 60%, 70%, 80%, 90%, 95%, 96%, 97%, 98%, 99% or 99.5% identical to the sequence shown in the respective line in Table II, application no. 18, columns 5 or 8.

[0352.1.7.18] to [0357.1.7.18] for the disclosure of these paragraphs see [0352.1.7.7] to [0357.1.7.7] above

Functional equivalents of the nucleic acid molecules according to the present invention and/or used in the process according to the present invention represent an embodiment of homologs. Functional equivalents derived from the nucleic acid molecules as shown in the respective line in Table I, application no. 18, columns 5 or 8, preferably the coding region thereof, according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the nucleic acid molecules as shown in the respective line in Table I, application no. 18, columns 5 or 8, preferably the coding region thereof, according to the invention and encode polypeptides having essentially the same properties as the polypeptide as shown in the respective line in Table II, application no. 18, columns 5 or 8.

Functional equivalents derived from one of the polypeptides as shown in the respective line in Table II, application no. 18, columns 5 or 8 according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the polypeptides as shown in the respective line in Table II, application no. 18, columns 5 or 8 according to the invention and having essentially the same properties as the polypeptide as shown in the respective line in Table II, application no. 18, columns 5 or 8.

for the disclosure of this paragraph see [0359.1.7.7] above

A nucleic acid molecule encoding a homolog to a polypeptide depicted in the respective line in Table II, application no. 18, columns 5 or 8 can be created by introducing one or more nucleotide substitutions, additions or deletions into the nucleic acid molecule of the present invention, in particular of Table I, application no. 18, columns 5 or 8, in the respective line, such that one or more amino acid substitutions, additions or deletions are introduced into the encoded protein. Mutations can be introduced into the nucleic acid molecules as depicted in the respective line in Table I, application no. 18, columns 5 or 8, preferably the coding region thereof, by standard techniques, such as site-directed mutagenesis, PCR-mediated mutagenesis or other methods known to the person skilled in the art.

[0361.1.7.18] to [0363.1.7.18] for the disclosure of these paragraphs see [0361.1.7.7] to [0363.1.7.7] above

Homologues of the nucleic acid sequences used, with the sequences shown in the respective line in Table I, application no. 18, columns 5 or 8, preferably the coding region thereof, comprise also allelic variants with at least approximately 30%, 35%, 40%, 45%, 50%, 55%, 60%, 65%, 70%, 75%, 80%, 85%, 90%, 91%, 92%, 93%, 94% 95%. 96%, 97%, 98%, 99%, 99.5% or more % homology with one of the nucleotide sequences shown or the above-mentioned derived nucleic acid sequences or their homologues or derivatives or parts of these. Allelic variants encompass in particular functional variants which can be obtained by deletion, insertion or substitution of nucleotides from the sequences shown in the respective line, preferably in Table I, columns 5 or 8, preferably the coding region thereof, or from the derived nucleic acid sequences, the intention being, however, that the enzymatic activity or the biological activity of the resulting proteins synthesized is advantageously retained or increased.

In one embodiment of the present invention, the nucleic acid molecule of the invention or used in the process of the invention comprises the sequences shown in the respective line in any of the Table I, application no. 18, columns 5 or 8, preferably the coding region thereof, fragments or homologs thereof. It is preferred that the nucleic acid molecule comprises as little as possible other nucleotides not shown in the respective line in any one of Table I, application no. 18, columns 5 or 8, preferably the coding region thereof, fragments or homologs thereof. In one embodiment, the nucleic acid molecule comprises less than 500, 400, 300, 200, 100, 90, 80, 70, 60, 50 or 40 further nucleotides. In a further embodiment, the nucleic acid molecule comprises less than 30, 20 or 10 further nucleotides. In one embodiment, the nucleic acid molecule used in the process of the invention is identical to the sequences shown in the respective line in Table I, application no. 18, columns 5 or 8, preferably the coding region thereof.

Also preferred is that the nucleic acid molecule used in the process of the invention encodes a polypeptide comprising the sequence shown in the respective line in Table II, application no. 18, columns 5 or 8, fragments or homologs thereof. In one embodiment, the nuoleic acid molecule encodes less than 150, 130, 100, 80, 60, 50, 40 or 30 further amino acids.

In a further embodiment, the encoded polypeptide comprises less than 20, 15, 10, 9, 8, 7, 6 or 5 further amino acids. In one embodiment used in the inventive process, the encoded polypeptide is identical to the sequences shown in the respective line in Table II, application no. 18, columns 5 or 8.

In one embodiment, the nucleic acid molecule of the invention or used in the process encodes a polypeptide comprising the sequence shown in the respective line in Table II, application no. 18, columns 5 or 8, or fragments or homologs thereof, and comprises less than 100 further nucleotides. In a further embodiment, said nucleic acid molecule comprises less than 30 further nucleotides. In one embodiment, the nucleic acid molecule used in the process is identical to a coding sequence of the sequences shown in the respective line in Table I, application no. 18, columns 5 or 8.

Polypeptides (=proteins), which still have the essential biological or enzymatic activity of the polypeptide of the present invention conferring the production or the increased production of the respective fine chemical cerotic acid, heneicosanoic acid, lignoceric acid, melissic acid or montanic acid as compared to a corresponding, e.g. non-transformed, wild type non-human organism, like a microorganism or a plant cell, plant or part thereof, i.e. whose activity is essentially not reduced, are polypeptides with at least 10%, 20%, 30%, 40%, 50%, 60%, 70%, 80%, 90%, 95% or more of the wild type biological activity or enzymatic activity, advantageously, the activity is essentially not reduced in comparison with the activity of a polypeptide shown in the respective line in Table II, application no. 18, columns 5 or 8 expressed under identical conditions.

Homologues of nucleic acid molecules shown in the respective line in Table I, application no. 18, columns 5 or 8, preferably the coding region thereof, or fragments thereof, or of the derived sequences shown in the respective line in Table II, application no. 18, columns 5 or 8, or fragments thereof, also mean truncated sequences, cDNA, single-stranded DNA or RNA of the coding and noncoding DNA sequence. Homologues of said sequences are also understood as meaning derivatives, which comprise noncoding regions such as, for example,

UTRs, introns, terminators, enhancers or promoter variants. The promoters upstream of the nucleotide sequences stated can be modified by one or more nucleotide substitution(s), insertion(s) and/or deletion(s) without, however, interfering with the functionality or activity either of the promoters, the open reading frame (=ORF) or with the 3′-regulatory region such as terminators or other 3′-regulatory regions, which are far away from the ORF. It is furthermore possible that the activity of the promoters is increased by modification of their sequence, or that they are replaced completely by more active promoters, even promoters from heterologous organisms. Appropriate promoters are known to the person skilled in the art and are mentioned herein below.

[0370.1.7.18] to [0379.1.7.18] for the disclosure of these paragraphs see [0370.1.7.7] to [0379.1.7.7] above

Moreover, a native polypeptide conferring the increase of the respective fine chemical cerotic acid, heneicosanoic acid, lignoceric acid, melissic acid or montanic acid in a non-human organism or a part thereof can be isolated from cells (e.g., endothelial cells), for example using the antibody of the present invention as described below, in particular, an antibody against proteins having 2,3-dihydroxy-2,3-dihydrophenylpropionatedehydrogenase, ABC metal ion transporter substrate-binding protein, acetyl-coenzyme A synthetase, acetyltransferase, acid shock protein, acyl transferase, agmatinase, aminotransferase, ATP-binding component of a transport system, b1522-protein, b3083-protein, b3644-protein, b3812-protein, b4029-protein, binding-protein-dependent transport systems inner membrane component, calcium-dependent protein kinase, cardiolipin synthase, chaperone protein CIpB, cyclin D, cystathionine gamma-synthase, eukaryotic translation initiation factor 5, geranylgeranyl pyrophosphate synthase, gibberellin 20-oxidase, glutamate-ammonia-ligase, glutaredoxin, glycosyl transferase, heat shock transcription factor, hydrolase, inositol-3-phosphate synthase, isochorismate synthase, leucyl/phenylalanyl-tRNA-protein transferase, major facilitator superfamily transporter protein, malate dehydrogenase, mevalonate kinase, mitochondrial inner membrane protease subunit, monodehydroascorbate reductase, multidrug resistance protein, NADH dehydrogenase subunit N, O-antigen chain length determinant, oxidoreductase, peroxisome assembly protein, potassium transport protein, protein kinase, purine nucleoside phosphorylase, rubredoxin, Sec-independent protein translocase subunit, serine protease, sll0064-protein, slr0383-protein, sodium/proton antiporter, thioredoxin family protein, threonine synthase, tryptophan synthase alpha chain, TTC0768-protein, TTC1386-protein, uroporphyrin-III C-methyltransferase, yer152c-protein, ylr065c-protein, yml093w-protein, or zinc finger protein activity, respectively, or an antibody against polypeptides as shown in the respective line in Table II, application no. 18, columns 5 or 8, or fragments or homologs thereof which can be produced by standard techniques utilizing the polypeptid of the present invention or fragment thereof, i.e., the polypeptide of this invention (FCRP). Preferred are monoclonal antibodies.

for the disclosure of this paragraph see [0381.1.7.7] above

In one embodiment, the present invention relates to a polypeptide as depicted in the respective line in Table II, especially in Table IIA or especially in Table IIB, application no. 18, columns 5 or 8, or fragment or homolog thereof or as coded by the nucleic acid molecule shown in the respective line in Table I, especially Table IA or especially Table IB, application no. 18, columns 5 or 8, preferably the coding region thereof, or fragment or homolog thereof.

In an embodiment, the present invention relates to a polypeptide comprising or consisting of a consensus sequence shown in the respective line in Table IV, application no. 18, column 8 or at least one polypeptide motif shown in the respective line in Table IV, application no. 18, column 8.

In an embodiment thereof, the present invention relates to a polypeptide comprising or consisting of a consensus sequence shown in the respective line in Table IV, application no. 18, column 8 or comprising at least one polypeptide motif shown in the respective line in Table IV, application no. 18, column 8, whereby not more than 20, 15, 10, 9, 8, 7, 6, 5, 4, 3, 2, or 1, or 0 of the amino acids can be replaced by any amino acid.

In another embodiment thereof, the present invention relates to a polypeptide comprising or consisting of a consensus sequence shown in the respective line in Table IV, application no. 18, column 8 or comprising at least one polypeptide motif shown in the respective line in Table IV, application no. 18, column 8, whereby not more than 15%, 10%, 5%, 4%, 3%, 2% orl% or 0% of the amino acids defined as distinct specific amino acids are/is replaced by another amino acid.

In another embodiment thereof, the present invention relates to a polypeptide comprising or consisting of a consensus sequence shown in the respective line in Table IV, application no. 18, column 8 or comprising at least one polypeptide motif shown in the respective line in Table IV, application no. 18, column 8, whereby not more than 20, 15, 10, 9, 8, 7, 6, 5, 4, 3, 2, or 1, or 0 amino acids are inserted into the consensus sequence or polypeptide motif.

In an embodiment of the present invention, the polypeptide of the present invention comprises or consists of a consensus sequence or at least one polypeptide motif shown in the respective line in Table IV, application no. 18, column 8, whereby said polypeptide distinguishes over the sequence depicted in the respective line in Table II, application no. 18, columns 5 or 8 by one or more amino acids. In one embodiment, said polypeptide of the invention distinguishes over the sequence shown in the respective line in Table II, application no. 18, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 18, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in the respective line in Table II, application no. 18, columns 5 and 8.

In an embodiment, the invention relates to a polypeptide conferring an increase in the respective fine chemical cerotic acid, heneicosanoic acid, lignoceric acid, melissic acid or montanic acid in a non-human organism, especially a microorganism or a plant, or a part thereof, being encoded by the nucleic acid molecule of the invention or used in the process of the invention and having a sequence which distinguishes over the sequence as shown in the respective line in Table II, application no. 18, columns 5 or 8 by one or more amino acids (but not exceeding 5%, preferably 4%, 3%, 2%, 1%, 0.5%, 0.2% or 0.1%). In an embodiment, said polypeptide of the invention does not comprise or consist of the sequence shown in the respective line in Table II, application no. 18, columns 5 or 8. In an embodiment, said polypeptide of the present invention is less than 100%, 99.999%, 99.99%, 99.9% or 99% identical. In one embodiment, said polypeptide which differs at least in one or more amino acids (but not exceeding 5%, preferably 4%, 3%, 2%, 1%, 0.5%, 0.2% or 0.1%) from the polypeptide shown in the respective line in Table II, application no. 18, columns 5 and 8 does not comprise a protein of the sequence shown in the respective line in Table II A and/or II B, application no. 18, columns 5 or 8.

In an embodiment, the present invention relates to a polypeptide having the activity of the protein as shown in the respective line in Table II, application no. 18, column 3, which distinguishes over the sequence depicted in the respective line in Table II, application no. 18, columns 5 or 8 by one or more amino acids, preferably by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 18, columns 5 and 8 by not more than 80%, 70%, 60%, 50%, 40%, 30%, 20%, 10%, 5%, 4%, 3%, 2%, 1% or 0.5%.

In another embodiment the polypeptide of the invention takes the form of a preprotein consisting of a plastidial or mitochondrial transit peptide joint to a polypeptide having the activity of the protein as shown in Table II, column 3, from which the transit peptide is preferably cleaved off upon transport of the preprotein into the organelle for example into the plastid or mitochondrion.

In another embodiment the polypeptide of the invention takes not the form of a preprotein.

for the disclosure of this paragraph see [0390.1.7.7] above

for the disclosure of this paragraph see [0391.1.7.7] above

A polypeptide of the invention can participate in the process of the present invention. The polypeptide or a portion thereof comprises preferably an amino acid sequence, which is sufficiently homologous to an amino acid sequence shown in the respective line in Table II A and/or II B, application no. 18, columns 5 or 8.

Further, the polypeptide can have an amino acid sequence which is encoded by a nucleotide sequence which hybridizes, preferably hybridizes under stringent conditions as described above, to a nucleotide sequence of the nucleic acid molecule of the present invention. Accordingly, the polypeptide has an amino acid sequence which is encoded by a nucleotide sequence that is at least about 35%, 40%, 45%, 50%, 55%, 60%, 65%, 70%, 75%, 80%, 85%, 90%, 91%, 92%, 93%, 94%, 95%, 96%, 97%, 98%, 99%, 99.5% or more homologous to one of the amino acid sequences as shown in the respective line in Table II A and/or II B, application no. 18, columns 5 or 8, or fragments thereof. The preferred polypeptide of the present invention preferably possesses at least one of the activities according to the invention and described herein. A preferred polypeptide of the present invention includes an amino acid sequence encoded by a nucleotide sequence which hybridizes, preferably hybridizes under stringent conditions, to a nucleotide sequence shown in the respective line in Table I A and/or I B, application no. 18, columns 5 or 8, preferably the coding region thereof, or fragments thereto or which is homologous thereto, as defined above

The polypeptide of the present invention can vary from the sequences shown in the respective line in Table II A and/or II B, application no. 18, columns 5 or 8 in amino acid sequence due to natural variation or mutagenesis, as described in detail herein. Accordingly, the polypeptide comprise an amino acid sequence which is at least about 35%, 40%, 45%, 50%, 55%, 60%, 65%, 70%, 75%, 80%, 85% or 90, 91%, 92%, 93%, 94%, 95%, 96%, 97%, 98%, 99%, 99.5% or more homologous to an entire amino acid sequence shown in the respective line in Table II A and/or II B, application no. 18, columns 5 or 8.

Biologically active portions of a polypeptide of the present invention include peptides comprising amino acid sequences derived from the amino acid sequence of the polypeptide of the present invention or used in the process of the present invention, e.g., the amino acid sequence shown in the respective line in Table II, application no. 18, columns 5 or 8 or the amino acid sequence of a protein homologous thereto, which include fewer amino acids than a full length polypeptide of the present invention or used in the process of the present invention or the full length protein which is homologous to a polypeptide of the present invention or used in the process of the present invention depicted herein, and exhibit at least one activity of a polypeptide of the present invention or used in the process of the present invention.

for the disclosure of this paragraph see [0396.1.7.7] above

Manipulation of the nucleic acid molecule of the invention may result in the production of a protein having differences from the protein as shown in the respective line in Table II, application no. 18, column 5 or 8. Differences shall mean at least one amino acid different from the sequences as shown in the respective line in Table II, application no. 18, column 5 or 8, preferably at least 2, 3, 4, 5, 6, 7, 8, 9 or 10 amino acids (especially but not exceeding 0.1%, preferably 0.2%, 0.5%, 1%, 2%, 3% 4% or 5%), more preferably at least 15, 20, 25, 30, 35, 40, 45 or 50 amino acids (especially but not exceeding 0.5%, preferably 1%, 2%, 3% 4%, 5%, 10% or 25%) different from the sequences as shown in the respective line in Table II, application no. 18, column 5 or 8. These proteins may be improved in efficiency or activity, or may be more stable and therefore present in greater numbers in the cell as compared to the wild-type cell.

Any mutagenesis strategies for the polypeptide of the present invention or the polypeptide used in the process of the present invention to result in increasing said activity are not meant to be limiting; variations on these strategies will be readily apparent to one skilled in the art. Using such strategies, and incorporating the mechanisms disclosed herein, the nucleic acid molecule and polypeptide of the invention may be utilized to generate algae, ciliates, fungi, other microorganisms like C. glutamicum or plants or parts thereof, expressing proteins as shown in the respective line in Table II, application no. 18, column 5 or 8, or mutated proteins thereof. The nucleic acid molecules and polypeptide molecules of the invention are expressed such that the yield, production, and/or efficiency of production of a desired compound is improved.

[0399.1.7.18] to [0401.1.7.18] for the disclosure of these paragraphs see [0399.1.7.7] to [0401.1.7.7] above

In one embodiment, a protein (=polypeptide) as shown in the respective line in Table II, application no. 18, column 5 or 8 refers to a polypeptide having an amino acid sequence corresponding to the polypeptide of the invention, especially to a polypeptide as shown in Table II, application no. 18, columns 5 or 8 or homologs thereof, or used in the process of the invention, whereas a “non- inventive protein or polypeptide” or “other polypeptide” refers to a polypeptide having an amino acid sequence corresponding to a protein which is not substantially homologous to a polypeptide of the invention, preferably which is not substantially homologous to a polypeptide or protein as shown in the respective line in Table II, application no. 18, column 5 or 8 or homologs thereof, e.g., a protein which does not confer the activity described herein and which is derived from the same or a different organism.

[0403.1.7.18] to [0409.1.7.18] for the disclosure of these paragraphs see [0403.1.7.7] to [0409.1.7.7] above

As the nucleic acid molecules of the invention and/or the expression thereof is related to the synthesis of the respective fine chemical cerotic acid, heneicosanoic acid, lignoceric acid, melissic acid or montanic acid its function as a probe extends to the detection of microorganisms, plant tissues, plants, plant variets, plant ecotypes or plant genera with varying, advantageously increased, capability or potential for synthesis of the respective fine chemical cerotic acid, heneicosanoic acid, lignoceric acid, melissic acid or montanic acid. Therefore in one embodiment the present invention relates to a method for analyzing the capability or potential of a plant tissue, a plant, a plant variety or ecotype to produce the respective fine chemical cerotic acid, heneicosanoic acid, lignoceric acid, melissic acid or montanic acid by using the respective antibody of the invention as a probe to detect the amount of the polypeptide encoded by said nucleic acid molecule of the invention in a non-human organism in comparison to another organism.

Therefore in one embodiment the invention relates to a method to probe a non-human organism for its capability for the production of the respective fine chemical comprising the steps of
a) analyzing the sequence and/or the expression of the respective nucleic acid molecule of the invention in least two non-human organisms which differ in the capability to produce the respective fine chemical;
b) associate the different sequence and/or expression level of the nucleic acid molecules of the invention with their different capability to produce the respective fine chemical;
c) analyze the sequence and/or the expression of the nucleic acid molecule of the invention in at least a third non-human organism;
d) predict the capability of the at least third organism to produce the respective fine chemical based by comparing the sequence and/or expression of the nucleic acid of the invention to the sequences and/or expression levels of the nucleic acid molecule of the invention of the at least two organisms analyzed in step a.

[0411.1.7.18] to [0430.1.7.18] for the disclosure of these paragraphs see [0411.1.7.7] to [0430.1.7.7] above

Accordingly the present invention relates to any cell transgenic for any nucleic acid characterized as part of the invention, e.g. conferring the increase of the respective fine chemical cerotic acid, heneicosanoic acid, lignoceric acid, melissic acid or montanic acid in a cell or a non-human organism or a part thereof, e.g. the nucleic acid molecule of the invention, the nucleic acid construct of the invention, the vector of the invention, the expression cassette according to the invention, or a nucleic acid molecule encoding the polypeptide of the invention, e.g. encoding a polypeptide having an activity as the protein as shown in the respective line in Table II, application no. 18, column 3. Due to the above-mentioned activity the respective fine chemical cerotic acid, heneicosanoic acid, lignoceric acid, melissic acid or montanic acid content in a cell or a non-human organism is increased. For example, due to modulation or manipulation, the cellular activity is increased, in a preferred embodiment in organelles such as plastids or mitochondria, e.g. due to an increased expression or specific activity or specific targeting of the subject matters of the invention in a cell or a non-human organism or a part thereof especially in organelles such as plastids or mitochondria, or in another embodiment in the cytosol. Transgenic for a polypeptide having a protein or a protein activity means herein that due to modulation or manipulation of the genome, the activity of protein as shown in the respective line in Table II, application no. 18, column 3 or a protein as shown in the respective line in Table II, application no. 18, column 3-like activity is increased in the cell or non-human organism or part thereof, especially in organelles such as plastids or mitochondria, or especially in the cytosol.

Examples are described above in context with the process of the invention.

for the disclosure of this paragraph see [0432.1.7.7] above

A naturally occurring expression cassette—for example the naturally occurring combination of the promoter of the gene encoding a protein as shown in the respective line in Table II, application no. 18, column 5 or 8, preferably the coding region thereof, or fragments or homologs thereof with the corresponding encoding gene—becomes a transgenic expression cassette when it is modified by non-natural, synthetic “artificial” methods such as, for example, mutagenesis. Such methods have been described (U.S. Pat. No. 5,565,350; WO 00/15815; also see above).

for the disclosure of this paragraph see [0434.1.7.7] above

for the disclosure of this paragraph see [0435.1.7.7] above

Transgenic plants comprising the respective fine chemical(s) synthesized in the process according to the invention can be marketed directly without isolation of the fine chemical synthesized. In the process according to the invention, plants are understood as meaning all plant parts, plant organs such as leaf, stalk, root, tubers or seeds or propagation material or harvested material or the intact plant. In this context, the seed encompasses all parts of the seed such as the seed coats, epidermal cells, seed cells, endosperm or embryonic tissue. The fine chemical produced in the process according to the invention may, however, also be isolated from the plant. In case of cerotic acid, heneicosanoic acid, lignoceric acid, melissic acid or montanic acid this can be in free form or bound to proteins. Fine chemical(s) produced by this process can be harvested by harvesting the non-human organisms either from the culture in which they grow or from the field. For example, this can be done via squeezing, grinding and/or extraction, salt precipitation and/or ion-exchange chromatography of the plant parts, preferably the plant seeds, plant fruits, plant tubers and the like.

[0437.1.7.18] to [0447.1.7.18] for the disclosure of these paragraphs see [0437.1.7.7] to [0447.1.7.7] above

The identity and purity of the isolated compound(s) can be determined by prior art techniques. These include high performance liquid chromatography (HPLC), spectroscopic methods, mass spectrometry (MS), staining methods, thin-layer chromatography, NIRS, enzyme assay or microbiological assays. These analytical methods are summarized for example in: Patek et al., Appl. Environ. Microbiol. 60, 133 (1994), Malakhova et al., Biotekhnologiya 11, 27 (1996); Schmidt et al., Bioprocess Engineer. 19, 67 (1998), Ullmann's Encyclopedia of Industrial Chemistry (1996) Vol. A27, VCH, Weinheim, pp. 89-90, pp. 521-540, pp. 540-547, pp. 559-566, 575-581 and pp. 581-587, Michal G., “Biochemical Pathways: An Atlas of Biochemistry and Molecular Biology”, John Wiley and Sons (1999); Fallon A. et al. “Applications of HPLC in Biochemistry” in “Laboratory Techniques in Biochemistry and Molecular Biology”, Vol. 17 (1987).

The fine chemical obtained in the process are suitable as starting material for the synthesis of further products of value.

Accordingly, the present invention relates a method for the production of a pharmaceuticals, food stuff, animal feeds, nutrients or cosmetics comprising the steps of the process according to the invention, including the isolation of the fine chemical produced or a composition produced comprising the fine chemical, and, if desired, formulating the product with a pharmaceutical acceptable carrier or formulating the product in a form acceptable for an application in agriculture or formulating the product in a form acceptable for an application as food stuff, animal feed, nutrient or cosmetic. A further embodiment according to the invention is the use of the fine chemical produced in the process or of the transgenic non-human organisms in animal feeds, foodstuffs, medicines, food supplements, cosmetics or pharmaceuticals.

[0450.1.7.18] to [0452.1.7.18] for the disclosure of these paragraphs see [0450.1.7.7] to [0452.1.7.7] above

Further in another embodiment, the present invention relates to the use of the nucleic acid molecule of the invention or used in the method of the invention alone or in combination with other genes of the respective fine chemical synthesis for example of the VLCFA biosynthesis, the polypeptide of the invention or used in the method of the invention, the nucleic acid construct of the invention, the expression cassette of the invention, the vector of the invention, the plant or plant tissue or the host cell of the invention, for the production of plant resistant to a herbicide inhibiting eventually the production of the fine chemical.

for the disclosure of this paragraph see [0454.1.7.7] above

In one embodiment, the present invention relates to a method for the identification of a gene product conferring the production of or an increase in the fine chemical production in a non-human cell, comprising the following steps:
(a) contacting, e.g. hybridizing, a, some or all nucleic acid molecules of a sample, e.g. cells, tissues, plants or microorganisms or a nucleic acid library, which can contain a candidate gene encoding a gene product conferring the production of or an increase in the fine chemical after expression, with the nucleic acid molecule of the present invention as shown in the respective line in column 5 or 8 of Table I A or B of application no. 18, preferably the coding region thereof, or a fragment or homolog thereof;
(b) identifying the nucleic acid molecules, which hybridize under relaxed stringent conditions with the nucleic acid molecule of the present invention in particular to the nucleic acid molecule sequence shown in the respective line in Table I, application no. 18, columns 5 or 8, preferably the coding region thereof, of a fragment or a homolog thereof, preferably in Table I B, application no. 18, columns 5 or 8, preferably the coding region thereof, of a fragment or a homolog thereof, and, optionally, isolating the full length cDNA clone or complete genomic clone;
(c) introducing the candidate nucleic acid molecules or a fragment thereof in host cells, preferably in a plant cell or a microorganism, appropriate for producing the fine chemical;
(d) expressing the identified nucleic acid molecules in the host cells for which the production of or the increased production of the fine chemical is desired;
(e) assaying the fine chemical level in the non-human host cells; and
(f) identifying the nucleic acid molecule and/or its gene product which expression confers the production of or an increase in the fine chemical level in the non-human host cell after expression compared to the wild type.

for the disclosure of this paragraph see [0456.1.7.7] above

In another embodiment, the present invention relates to a method for the identification of a gene product conferring the production of or an increase in the fine chemical production in a cell, comprising the following steps:
(a) identifying a nucleic acid molecule of a non-human organism, which is at least 20%, 25%, 30%, 35%, 40%, 50%, 60%, 70%, 80%, 90%, 95%, 96%, 97%, 98%, 99%, 99.5% or more homolog to the nucleic acid molecule encoding a polypeptide comprising the polypeptide molecule as shown in the respective line in column 5 or 8 of Table II, application no. 18, or comprising a consensus sequence or a polypeptide motif as shown in the respective line in column 8 of Table IV, application no. 18, or being encoded by a nucleic acid molecule comprising a polynucleotide as shown in the respective line in column 5 or 8 of Table I, application no. 18, preferably the coding region thereof, or a homologue thereof as described herein, for example via homology search in a data bank;
(b) introducing the candidate nucleic acid molecule in non-human host cells, preferably in a plant cell or a microorganism, appropriate for producing the fine chemical;
(c) expressing the identified nucleic acid molecule in the non-human host cells;
(d) assaying the fine chemical level in the non-human host cells; and
(e) identifying the nucleic acid molecule and its gene product which expression confers the production of or an increase in the fine chemical level in the non-human host cell after expression compared to the wild type.

for the disclosure of this paragraph see [0458.1.7.7] above

Further, the nucleic acid molecules disclosed herein, in particular the nucleic acid molecules shown in the respective line in column 5 or 8 of Table I A or B of application no. 18, may be sufficiently homologous to the sequences of related species such that these nucleic acid molecules may serve as markers for the construction of a genomic map in related organism or for association mapping. Furthermore natural variation in the genomic regions corresponding to nucleic acids disclosed herein, in particular the nucleic acid molecule shown in the respective line in column 5 or 8 of Table I A or B, of application no. 18 or homologous thereof, may lead to variation in the activity of the proteins disclosed herein, in particular the proteins comprising polypeptides as shown in the respective lines in column 5 or 8 of Table II A or B, of application no. 18 or their homologous, or comprising the consensus sequence or the polypeptide motif as shown in the respective line in column 8 of Table IV, and in consequence in a natural variation of an increased fine chemical production.

In consequence natural variation eventually also exists in form of more active allelic variants leading already to a relative increase in the fine chemical. Different variants of the nucleic acid molecules disclosed herein, in particular the nucleic acid molecules comprising the nucleic acid molecules as shown in the respective line in column 5 or 8 of Table I A or B of application no. 18, preferably the coding region thereof, which corresponds to different levels of increase in fine chemical, e.g. different levels of increase in fine chemical, can be indentified and used for marker assisted breeding for an enhanced production of the fine chemical.

Natural variation may also exist in the regulatory regions, e.g. the promoters of the natural genes comprising the nucleic acid molecules as shown in the respective line column 5 or 8 of Table I A or B of application no. 18, preferably the coding region thereof, which can similarly correspond to different levels of increase in the fine chemical, e.g. different levels of increase in the fine chemical can be identified and used for marker assisted breeding for an enhanced production of the fine chemical.

Accordingly, the present invention relates to a method for breeding plants for the production of the fine chemical, comprising
(a) selecting a first plant variety with an increased production of the fine chemical, e.g. based on an increased expression of a nucleic acid molecule of the invention as disclosed herein, in particular of a nucleic acid molecule comprising a nucleic acid molecule as shown in the respective line in column 5 or 8 of Table I A or B of application no. 18, preferably the coding region thereof, or fragments or homologs thereof, or a polypeptide comprising a polypeptide as shown in the respective line in column 5 or 8 of Table II A or B of application no. 18, or fragments or homologs thereof, or comprising a consensus sequence or a polypeptide motif as shown in the respective line in column 8 of Table IV, or a homolog thereof, as described herein;
(b) associating the level of increased production of the fine chemical, with the expression level or the genomic structure of a gene encoding said polypeptide or said nucleic acid molecule;
(c) crossing the first plant variety with a second plant variety, which significantly differs in its level of fine chemical; and
(d) identifying, which of the offspring varieties has got increased production of the fine chemical by the expression level of said polypeptide or nucleic acid molecule or the genomic structure of the gene(s) encoding said polypeptide or nucleic acid molecule of the invention.

[0462.1.7.18] to [0479.1.7.18] for the disclosure of these paragraphs see [0462.1.7.7] to [0479.1.7.7] above

In a further embodiment the nucleic acid molecule of the invention, the polypeptide of the invention, the nucleic acid construct of the invention, the expression cassette of the invention, the non-human organisms, the non-human host cell, the microorganisms, the plant, plant cell, or the part thereof, of the invention, the vector of the invention, the agonist identified with the method of the invention, the nucleic acid molecule identified with the method of the present invention, can be used for the production of the fine chemical or of the fine chemical and one or more other waxes and/or VLCFA.

Accordingly, the nucleic acid molecule of the invention, or the nucleic acid molecule identified with the method of the present invention or the complement sequences thereof, the polypeptide of the invention, the nucleic acid construct of the invention, the expression cassette of the invention, the non-human organism, the non-human host cell, the microorganism, the plant, plant cell, or the part thereof of the invention, the vector of the invention, the agonist identified with the method of the invention, the antibody of the present invention, can be used for the production of or the increase in the fine chemical in a non-human organism or part thereof, e.g. in a cell.

for the disclosure of this paragraph see [0481.1.7.7] above

for the disclosure of this paragraph see [0482.1.7.7] above

A special embodiment of this invention is related to genes which increase or generate the production of the fine chemical cerotic acid, heneicosanoic acid, lignoceric acid, melissic acid and/or montanic acid in plant cells, plants or part thereof. Phenotypes thereto are associated with yield of plants (=yield related phenotypes). In accordance with the invention, therefore, the respective genes identified in Table I, wherein in column 7 cerotic acid, heneicosanoic acid, lignoceric acid, melissic acid and/or montanic acid are mentioned, especially the coding region thereof, or homologs or fragments thereof,may be employed to enhance any yield-related phenotype. Increased yield may be determined in field trials of transgenic plants and suitable control plants. Alternatively, a transgene's ability to increase yield may be determined in a model plant. An increased yield phenotype may be determined in the field test or in a model plant by measuring any one or any combination of the following phenotypes, in comparison to a control plant: yield of dry harvestable parts of the plant, yield of dry aerial harvestable parts of the plant, yield of underground dry harvestable parts of the plant, yield of fresh weight harvestable parts of the plant, yield of aerial fresh weight harvestable parts of the plant yield of underground fresh weight harvestable parts of the plant, yield of the plant's fruit (both fresh and dried), grain dry weight, yield of seeds (both fresh and dry), and the like.

The most basic yield-related phenotype is increased yield associated with the presence of the gene or a homolog or a fragment thereof as a transgene in the plant, i.e., the intrinsic yield of the plant. Intrinsic yield capacity of a plant can be, for example, manifested in a field test or in a model system by demonstrating an improvement of seed yield (e.g. in terms of increased seed/grain size, increased ear number, increased seed number per ear, improvement of seed filling, improvement of seed composition, embryo and/or endosperm improvements, and the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under non-stressed conditions), improvement in plant architecture.

Increased yield-related phenotypes may also be measured to determine tolerance to abiotic environmental stress. Abiotic stresses include drought, low temperature, salinity, osmotic stress, shade, high plant density, mechanical stresses, and oxidative stress, and yield-related phenotypes are encompassed by tolerance to such abiotic stresses. Additional phenotypes that can be monitored to determine enhanced tolerance to abiotic environmental stress include, without limitation, wilting; leaf browning; loss of turgor, which results in drooping of leaves or needles stems, and flowers; drooping and/or shedding of leaves or needles; the leaves are green but leaf angled slightly toward the ground compared with controls; leaf blades begun to fold (curl) inward; premature senescence of leaves or needles; loss of chlorophyll in leaves or needles and/or yellowing. Any of the yield-related phenotypes described above may be monitored in field tests or in model plants to demonstrate that a transgenic plant has increased tolerance to abiotic environmental stress. In accordance with the invention, the respective genes identified in Table 1, in case in column 7 cerotic acid, heneicosanoic acid, lignoceric acid, melissic acid and/or montanic acid is indicated, especially the coding region thereof, or homologs or fragments thereof, may be employed to enhance tolerance to abiotic environmental stress in a plant means that the plant, when confronted with abiotic environmental stress.

A polypeptide conferring an yield-increasing activity can be encoded by a respective nucleic acid sequence as shown in Table I, column 5 or 8, and/or comprises or consists of a respective polypeptide as depicted in Table II, column 5 and 8, and/or can be amplified with the respective primer set shown in Table III, column 8, in case in column 7 cerotic acid, heneicosanoic acid, lignoceric acid, melissic acid and/or montanic acid is indicated.

“Improved adaptation” to environmental stress like e.g. freezing and/or chilling temperatures refers to an improved plant performance under environmental stress conditions.

A modification, i.e. an increase, can be caused by endogenous or exogenous factors. For example, an increase in activity in an organism or a part thereof can be caused by adding a gene product or a precursor or an activator or an agonist to the media or nutrition or can be caused by introducing said subjects into an organism, transient or stable. Furthermore such an increase can be reached by the introduction of the respective inventive nucleic acid sequence or the encoded protein in the correct cell compartment for example into the nucleus or cytoplasmic respectively or into plastids either by transformation and/or targeting.

The term “yield” as used herein generally refers to a measurable produce from a plant, particularly a crop. Yield and yield increase (in comparison to a non-transformed starting or wild-type plant) can be measured in a number of ways, and it is understood that a skilled person will be able to apply the correct meaning in view of the particular embodiments, the particular crop concerned and the specific purpose or application concerned. The terms “improved yield” or “increased yield” can be used interchangeable.

As used herein, the term “improved yield” or the term “increased yield” means any improvement in the yield of any measured plant product, such as grain, fruit or fiber. In accordance with the invention, changes in different phenotypic traits may improve yield. For example, and without limitation, parameters such as floral organ development, root initiation, root biomass, seed number, seed weight, harvest index, tolerance to abiotic environmental stress, leaf formation, phototropism, apical dominance, and fruit development, are suitable measurements of improved yield. Any increase in yield is an improved yield in accordance with the invention. For example, the improvement in yield can comprise a 0.1%, 0.5%, 1%, 3%, 5%, 10%, 15%, 20%, 30%, 40%, 50%, 60%, 70%, 80%, 90% or greater increase in any measured parameter. For example, an increase in the bushel/acre yield of soybeans or corn derived from a crop comprising plants which are transgenic for the nucleotides and polypeptides of Table I, in case in column 7 cerotic acid, heneicosanoic acid, lignoceric acid, melissic acid and/or montanic acid is mentioned, as compared with the bushel/acre yield from untreated soybeans or corn cultivated under the same conditions, is an improved yield in accordance with the invention. The increased or improved yield can be achieved in the absence or presence of stress conditions.

For example, enhanced or increased “yield” refers to one or more yield parameters selected from the group consisting of biomass yield, dry biomass yield, aerial dry biomass yield, underground dry biomass yield, fresh-weight biomass yield, aerial fresh-weight biomass yield, underground fresh-weight biomass yield; enhanced yield of harvestable parts, either dry or fresh-weight or both, either aerial or underground or both; enhanced yield of crop fruit, either dry or fresh-weight or both, either aerial or underground or both; and preferably enhanced yield of seeds, either dry or fresh-weight or both, either aerial or underground or both.

Crop yield is defined herein as the number of bushels of relevant agricultural product (such as grain, forage, or seed) harvested per acre. Crop yield is impacted by abiotic stresses, such as drought, heat, salinity, and cold stress, and by the size (biomass) of the plant. Traditional plant breeding strategies are relatively slow and have in general not been successful in conferring increased tolerance to abiotic stresses. Grain yield improvements by conventional breeding have nearly reached a plateau in maize.

Accordingly, the yield of a plant can depend on the specific plant/crop of interest as well as its intended application (such as food production, feed production, processed food production, biofuel, biogas or alcohol production, or the like) of interest in each particular case. Thus, in one embodiment, yield is calculated as harvest index (expressed as a ratio of the weight of the respective harvestable parts divided by the total biomass), harvestable parts weight per area (acre, square meter, or the like); and the like. The harvest index, i.e., the ratio of yield biomass to the total cumulative biomass at harvest, in maize has remained essentially unchanged during selective breeding for grain yield over the last hundred years. Accordingly, recent yield improvements that have occurred in maize are the result of the increased total biomass production per unit land area. This increased total biomass has been achieved by increasing planting density, which has led to adaptive phenotypic alterations, such as a reduction in leaf angle, which may reduce shading of lower leaves, and tassel size, which may increase harvest index. Harvest index is relatively stable under many environmental conditions, and so a robust correlation between plant size and grain yield is possible. Plant size and grain yield are intrinsically linked, because the majority of grain biomass is dependent on current or stored photosynthetic productivity by the leaves and stem of the plant. As with abiotic stress tolerance, measurements of plant size in early development, under standardized conditions in a growth chamber or greenhouse, are standard practices to measure potential yield advantages conferred by the presence of a transgene.

Acccordingly, the yield of a plant can be increased by improving one or more of the yield-related phenotypes.

Such yield-related phenotypes or traits of a plant the improvement of which results in increased yield comprise, without limitation, the increase of the intrinsic yield capacity of a plant, improved nutrient use efficiency, and/or increased stress tolerance.

For example, yield refers to biomass yield, e.g. to dry weight biomass yield and/or fresh-weight biomass yield. Biomass yield refers to the aerial or underground parts of a plant, depending on the specific circumstances (test conditions, specific crop of interest, application of interest, and the like). In one embodiment, biomass yield refers to the aerial and underground parts. Biomass yield may be calculated as fresh-weight, dry weight or a moisture adjusted basis. Biomass yield may be calculated on a per plant basis or in relation to a specific area (e.g. biomass yield per acre/square meter/ or the like).

In other embodiment, “yield” refers to seed yield which can be measured by one or more of the following parameters: number of seeds or number of filled seeds (per plant or per area (acre/square meter/ or the like)); seed filling rate (ratio between number of filled seeds and total number of seeds); number of flowers per plant; seed biomass or total seeds weight (per plant or per area (acre/square meter/ or the like); thousand kernel weight (TKW; extrapolated from the number of filled seeds counted and their total weight; an increase in TKW may be caused by an increased seed size, an increased seed weight, an increased embryo size, and/or an increased endosperm). Other parameters allowing to measure seed yield are also known in the art. Seed yield may be determined on a dry weight or on a fresh weight basis, or typically on a moisture adjusted basis, e.g. at 15.5 percent moisture.

For example, the term “increased yield” means that the plant, exhibits an increased growth rate, under conditions of abiotic environmental stress, compared to the corresponding wild-type plant.

An increased growth rate may be reflected inter alia by or confers an increased biomass production of the whole plant, or an increased biomass production of the aerial parts of a plant, or by an increased biomass production of the underground parts of a plant, or by an increased biomass production of parts of a plant, like stems, leaves, blossoms, fruits, and/or seeds.

Increased yield includes higher fruit yields, higher seed yields, higher fresh matter production, and/or higher dry matter production.

The term “increased yield” means that the plant, exhibits a prolonged growth under conditions of abiotic environmental stress, as compared to the corresponding, e.g. non-transformed, wild type organism. A prolonged growth comprises survival and/or continued growth of the plant, at the moment when the non-transformed wild type organism shows visual symptoms of deficiency and/or death.

When the plant of the invention is a corn plant increased yield for corn plants means, for example, increased seed yield, in particular for corn varieties used for feed or food. Increased seed yield of corn refers to an increased kernel size or weight, an increased kernel per ear, or increased ears per plant. Alternatively or in addition the cob yield may be increased, or the length or size of the cob is increased, or the kernel per cob ratio is improved.

When the plant of the invention is a soy plant, increased yield for soy plants means increased seed yield, in particular for soy varieties used for feed or food. Increased seed yield of soy refers for example to an increased kernel size or weight, an increased kernel per pod, or increased pods per plant.

When the plant of the invention is an oil seed rape (OSR) plant, increased yield for OSR plants means increased seed yield, in particular for OSR varieties used for feed or food. Increased seed yield of OSR refers to an increased seed size or weight, an increased seed number per silique, or increased siliques per plant.

When the plant of the invention is a cotton plant, increased yield for cotton plants means increased lint yield. Increased lint yield of cotton refers in one embodiment to an increased length of lint.

When the plant of the invention is a wheat plant increased yield for wheat plants means, for example, increased seed yield, in particular for wheat varieties used for feed or food. Increased seed yield of wheat refers to an increased kernel size or weight, an increased kernel per ear, or increased ears per plant. Increased seed yield of wheat refers also to an increased grain size or weight, an increased number of grains per spike, or increased number of spikes per plant. Alternatively or in addition increased seed yield of wheat refers also to an increased grain yield, increased length of spikes, increased number of spikelets per spike , increased 1000 grain weight or increased flag leaf area (flag leaf is the leaf directely below the spike).

Said increased yield can typically be achieved by enhancing or improving, one or more yield-related traits of the plant. Such yield-related traits of a plant comprise, without limitation, the increase of the intrinsic yield capacity of a plant, improved nutrient use efficiency, and/or increased stress tolerance, in particular increased abiotic stress tolerance. Intrinsic yield capacity of a plant can be, for example, manifested by improving the specific (intrinsic) seed yield (e.g. in terms of increased seed/ grain size, increased ear number, increased seed number per ear, improvement of seed filling, improvement of seed composition, embryo and/or endosperm improvements, or the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under stressed or non-stressed conditions), improvement in plant architecture, cell cycle modifications, photosynthesis modifications, various signaling pathway modifications, modification of transcriptional regulation, modification of translational regulation, modification of enzyme activities, and the like); and/or the like.

The improvement or increase of stress tolerance of a plant can for example be manifested by improving or increasing a plant's tolerance against stress, particularly abiotic stress. In the present application, abiotic stress refers generally to abiotic environmental conditions a plant is typically confronted with, including, but not limited to, drought (tolerance to drought may be achieved as a result of improved water use efficiency), heat, low temperatures and cold conditions (such as freezing and chilling conditions), salinity, osmotic stress , shade, high plant density, mechanical stress, oxidative stress, and the like.

The increased plant yield can also be mediated by increasing the “nutrient use efficiency of a plant”, e.g. by improving the use efficiency of nutrients including, but not limited to, phosphorus, potassium, and nitrogen. Further, higher yields may be obtained with current or standard levels of nitrogen use.

Generally, the term “increased tolerance to stress” can be defined as survival of plants, and/or higher yield production, under stress conditions as compared to a non-transformed wild type or starting plant: For example, the plant of the invention or produced according to the method of the invention is better adapted to the stress conditions. “Improved adaptation” to environmental stress like e.g. drought, heat, nutrient depletion, freezing and/or chilling temperatures refers herein to an improved plant performance resulting in an increased yield, particularly with regard to one or more of the yield related traits as defined in more detail above.

During its life-cycle, a plant is generally confronted with a diversity of environmental conditions. Any such conditions, which may, under certain circumstances, have an impact on plant yield, are herein referred to as “stress” condition. Environmental stresses may generally be divided into biotic and abiotic (environmental) stresses. Unfavorable nutrient conditions are sometimes also referred to as “environmental stress”. The present invention does also contemplate solutions for this kind of environmental stress, e.g. referring to increased nutrient use efficiency.

For the purposes of the description of the present invention, the terms “enhanced tolerance to abiotic stress”, “enhanced resistance to abiotic environmental stress”, “enhanced tolerance to environmental stress”, “improved adaptation to environmental stress” and other variations and expressions similar in its meaning are used interchangeably and refer, without limitation, to an improvement in tolerance to one or more abiotic environmental stress(es) as described herein and as compared to a corresponding origin or wild type plant or a part thereof.

The term abiotic stress tolerance(s) refers for example low temperature tolerance, drought tolerance or improved water use efficiency (WUE), heat tolerance, salt stress tolerance and others. Studies of a plant's response to desiccation, osmotic shock, and temperature extremes are also employed to determine the plant's tolerance or resistance to abiotic stresses. Water use efficiency (WUE) is a parameter often correlated with drought tolerance. In selecting traits for improving crops, a decrease in water use, without a change in growth would have particular merit in an irrigated agricultural system where the water input costs were high. An increase in growth without a corresponding jump in water use would have applicability to all agricultural systems. In many agricultural systems where water supply is not limiting, an increase in growth, even if it came at the expense of an increase in water use also increases yield.

Drought stress means any environmental stress which leads to a lack of water in plants or reduction of water supply to plants, including a secondary stress by low temperature and/or salt, and/or a primary stress during drought or heat, e.g. desiccation etc.

### Specific Embodiments

Accordingly, this invention provides respective measures and methods to produce plants with increased yield, e.g. genes conferring an increased yield-related trait, for example enhanced tolerance to abiotic environmental stress, for example an increased drought tolerance and/or low temperature tolerance and/or an increased nutrient use efficiency, intrinsic yield and/or another increased yield-related trait, upon expression or over-expression. Accordingly, the present invention provides such genes in case in column 7 of the respective Table or cerotic acid, heneicosanoic acid, lignoceric acid, melissic acid and/or montanic acid is indicated. In particular, such genes are described in column 5 as well as in column 8 of Tables I, especially the coding region thereof, or homologs or fragments thereof, in case cerotic acid, heneicosanoic acid, lignoceric acid, melissic acid and/or montanic acid are indicated in column 7 or the respective polypeptides are described in column 5 as well as in column 8 of Table II, or homologs or fragments thereof, in case cerotic acid, heneicosanoic acid, lignoceric acid, melissic acid and/or montanic acid are indicated in column 7.

Accordingly, the present invention provides respective transgenic plants showing one or more improved yield-related traits as compared to the corresponding origin or the wild type plant and methods for producing such transgenic plants with increased yield in case in column 7 of the respective Table “cerotic acid” or “heneicosanoic acid” or “lignoceric acidcid” or “melissic acid” or “montanic acid” is indicated.

In one embodiment, one or more of said yield-increasing activities are increased by increasing the amount and/or the specific activity of one or more proteins listed in Table II, column 5 or 8 in a compartment of a cell indicated in Table II, column 6, in case in column 7 of the respective Table “cerotic acid” or “heneicosanoic acid” or “lignoceric acidcid” or “melissic acid” or “montanic acid” is indicated.

Accordingly to present invention, the yield of the plant of the invention is increased by improving one or more of the yield-related traits as defined herein. Said increased yield in accordance with the present invention can typically be achieved by enhancing or improving, in comparison to an origin or wild-type plant, one or more yield-related traits of said plant. Such yield-related traits of a plant the improvement of which results in increased yield comprise, without limitation, the increase of the intrinsic yield capacity of a plant, improved nutrient use efficiency, like nitrogen use efficiency, and/or increased stress tolerance.

In one embodiment, the present invention provides respective transgenic plants showing one or more improved yield-related traits, preferably increased drought tolerance as compared to the corresponding origin or the wild type plant and methods for producing such transgenic plants with increased yield in case in column 7 of the respective Table “cerotic acid” or “heneicosanoic acid” or “lignoceric acidcid” or “melissic acid” or “montanic acid” is indicated.

In a further embodiment the present invention is characterized by the features as disclosed in the following items:

Item 1. A process for the production of a fine chemical selected from the group consisting of: cerotic acid, heneicosanoic acid, lignoceric acid, melissic acid and/or montanic acid
(a) increasing or generating one or more activities selected from the group consisting of 2,3-dihydroxy-2,3-dihydrophenylpropionatedehydrogenase, ABC metal ion transporter substrate-binding protein, acetyl-coenzyme A synthetase, acetyltransferase, acid shock protein, acyl transferase, agmatinase, aminotransferase, ATP-binding component of a transport system, b1522-protein, b3083-protein, b3644-protein, b3812-protein, b4029-protein, binding-protein-dependent transport systems inner membrane component, calcium-dependent protein kinase, cardiolipin synthase, chaperone protein CIpB, cyclin D, cystathionine gamma-synthase, eukaryotic translation initiation factor 5, geranylgeranyl pyrophosphate synthase, gibberellin 20-oxidase, glutamate-ammonia-ligase, glutaredoxin, glycosyl transferase, heat shock transcription factor, hydrolase, inositol-3-phosphate synthase, isochorismate synthase, leucyl/phenylalanyl-tRNA-protein transferase, major facilitator superfamily transporter protein, malate dehydrogenase, mevalonate kinase, mitochondrial inner membrane protease subunit, monodehydroascorbate reductase, multidrug resistance protein, NADH dehydrogenase subunit N, O-antigen chain length determinant, oxidoreductase, peroxisome assembly protein, potassium transport protein, protein kinase, purine nucleoside phosphorylase, rubredoxin, Sec-independent protein translocase subunit, serine protease, sll0064-protein, slr0383-protein, sodium/proton antiporter, thioredoxin family protein, threonine synthase, tryptophan synthase alpha chain, TTC0768-protein, TTC1386-protein, uroporphyrin-III C-methyltransferase, yer152c-protein, ylr065c-protein, yml093w-protein, and zinc finger protein, in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of cerotic acid, heneicosanoic acid, lignoceric acid, melissic acid and/or montanic acid or a composition comprising cerotic acid, heneicosanoic acid, lignoceric acid, melissic acid and/or montanic acid in said non-human organism or in the culture medium surrounding said non-human organism.

Item 2. A process for the production of a a fine chemical selected from the group consisting of: cerotic acid, heneicosanoic acid, lignoceric acid, melissic acid and/or montanic acid, which comprises
(A)
   (i) increasing or generating of the expression of; and/or
   (ii) increasing or generating the expression of an expression product of; and/or
   (iii) increasing or generating one or more activities of an expression product encoded by;
      at least one respective nucleic acid molecule comprising a nucleic acid molecule selected from the group consisting of:
   (a) a nucleic acid molecule encoding the polypeptide shown in column 5 or 8 of Table II, preferably Table II B, application no.18, or a homolog or a fragment thereof;
   (b) a nucleic acid molecule shown in column 5 or 8 of Table I, preferably Table I B, application no.18, or a homolog or a fragment thereof (preferably the coding region thereof);
   (c) a nucleic acid molecule, which, as a result of the degeneracy of the genetic code, can be derived from a polypeptide sequence depicted in column 5 or 8 of Table II, preferably Table II B, application no.18;
   (d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 18, or the coding region thereof;
   (e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
   (f) a nucleic acid molecule which hybridizes with a nucleic acid molecule of (a), (b), (c), (d) or (e) under stringent hybridization conditions;
   (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   (h) a nucleic acid molecule encoding a polypeptide comprising the consensus sequence or one or more polypeptide motifs as shown in column 8 of Table IV, application no.18;
   (i) a nucleic acid molecule encoding a polypeptide having the activity represented by a respective protein as depicted in column 5 of Table II, application no. 18;
   (j) a nucleic acid molecule which comprises a polynucleotide, which is obtained by amplifying a cDNA library or a genomic library using the primers as depicted in column 8 of Table III, application no. 18; and
   (k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
   or a nucleic acid molecule comprising a sequence which is complementary thereto;
      in a non-human organism or a part thereof, preferably a microorganism, a plant cell, a plant or a part thereof, as compared to a corresponding non-transformed wild type non-human organism or a part thereof; and
(B) growing the non-human organism or a part thereof under conditions which permit the production of cerotic acid, heneicosanoic acid, lignoceric acid, melissic acid and/or montanic acid or a composition comprising cerotic acid, heneicosanoic acid, lignoceric acid, melissic acid and/or montanic acid in said non-human organism or in the culture medium surrounding said non-human organism.

Item 3. A process of items 1 or 2, comprising of recovering cerotic acid, heneicosanoic acid, lignoceric acid, melissic acid and/or montanic acid in its free or bound form.

Item 4. The process of any one of items 1 to 3, comprising the following steps:
(a) selecting a non-human organism or a part thereof expressing a polypeptide encoded by the nucleic acid molecule characterized in item 2;
(b) mutagenizing the selected non-human organism or the part thereof;
(c) comparing the activity or the expression level of said polypeptide in the mutagenized non-human organism or the part thereof with the activity or the expression of said polypeptide of the selected non-human organisms or the part thereof;
(d) selecting the mutated non-human organisms or parts thereof, which comprise an increased activity or expression level of said polypeptide compared to the selected non-human organism or the part thereof;
(e) optionally, growing and cultivating the non-human organisms or the parts thereof; and
(f) recovering, and optionally isolating, the free or bound cerotic acid, heneicosanoic acid, lignoceric acid, melissic acid and/or montanic acid produced by the selected mutated non-human organisms or parts thereof.

Item 5. The process of any one of items 1 to 4 wherein the activity of said protein or the expression of said nucleic acid molecule is increased or generated transiently or stably.

Item 6. An isolated nucleic acid molecule comprising a nucleic acid molecule selected from the group consisting of:
(a) a nucleic acid molecule encoding a polypeptide shown in Table II, application no. 18, column 5 or 8, preferably shown in Table II B, application no. 18, column 8, or a homolog or a fragment thereof;
(b) a nucleic acid molecule shown in Table I, application no. 18, column 5 or 8, preferably shown in Table I B, application no. 18, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, application no. 18, preferably in column 8 of Table II B, application no. application no.;
(d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 3, preferably shown in column 8 of Table I B, application no. 18, preferably the coding region thereof;
(e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridisation conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 18;
(i) a nucleic acid molecule encoding a polypeptide having the activity represented by a respective protein as depicted in column 5 of Table II, application no. 18;
(j) a nucleic acid molecule comprising a polynucleotide which is obtained by amplifying a cDNA library or a genomic library using the primers in Table III, application no. 18, column 8;
and
(k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (i);
or a nucleic acid molecule comprising a sequence which is complementary thereto.

Item 7. A nucleic acid construct which confers the expression of the nucleic acid molecule as defined in item 6, comprising one or more regulatory elements.

Item 8. An expression cassette comprising
(a) a promoter, preferably selected from the group consisting of Big35S, PCUbi, Super and USP;
(b) a nucleic acid molecule as defined in item 6.

Item 9. A vector comprising the nucleic acid molecule as defined in item 6 or the nucleic acid construct as claimed in item 7 or the expression cassette as claimed in item 8.

Item 10. A host cell or a plant organelle, which has been transformed with the vector as claimed in item 9 or the nucleic acid molecule as defined in item 6 or the nucleic acid construct as claimed in item 7 or the expression cassette as claimed in item 8.

Item 11. The host cell of item 10, which is a plant cell or a microorganism.

Item 12. A process for producing a polypeptide, wherein the polypeptide is expressed in a host cell as claimed in item 10 or 11.

Item 13. A polypeptide produced by the process as claimed in item 12 or encoded by the nucleic acid molecule as defined in item 6.

Item 14. An antibody, which binds specifically to the polypeptide as claimed in item 13.

Item 15. A plant tissue, propagation material, harvested material or a plant comprising the host cell as claimed in item 10 or 11.

Item 16. A process for the identification of a compound conferring an increase in cerotic acid, heneicosanoic acid, lignoceric acid, melissic acid and/or montanic acid production in a non-human organism, comprising the steps:
(a) culturing a plant cell or tissue or microorganism or maintaining a plant expressing the polypeptide encoded by the nucleic acid molecule as defined in item 6 conferring an increase in the amount of cerotic acid heneicosanoic acid, lignoceric acid, melissic acid, and/or montanic acid in a non-human organism or a part thereof and a readout system capable of interacting with the polypeptide under suitable conditions which permit the interaction of the polypeptide with said readout system in the presence of a compound or a sample comprising a plurality of compounds and capable of providing a detectable signal in response to the binding of a compound to said polypeptide under conditions which permit the expression of said readout system and of the polypeptide encoded by the nucleic acid molecule of item 6 conferring an increase in the amount of cerotic acid, heneicosanoic acid, lignoceric acid, melissic acid and/or montanic acidin a non-human organism or a part thereof;
(b) identifying if the compound is an effective agonist by detecting the presence or absence or increase of a signal produced by said readout system.

Item 17. A method for the production of an agricultural composition comprising the steps of the method of item 16 and formulating the compound identified in item 16 in a form acceptable for an application in agriculture.

Item 18. A composition comprising the nucleic acid molecule as defined in item 6, the polypeptide as claimed in item 13, the nucleic acid construct as claimed in item 7, the expression cassette as claimed in item 8, the vector as claimed in item 9, the compound as claimed in item 16, the antibody as claimed in item 14, and optionally an agricultural acceptable carrier.

Item 19. Use of the nucleic acid molecule as defined in item 6 for the identification of a nucleic acid molecule conferring an increase in cerotic acid, heneicosanoic acid, lignoceric acid, melissic acid and/or montanic acid after expression.

Item 20. Cosmetic, pharmaceutical, food or feed composition comprising the nucleic acid moleculeas defined in item 6, the polypeptide as claimed in item 13, the nucleic acid construct as claimed in item 7, the expression cassette as claimed in item 8, the vector as claimed in item 9, the antibody as claimed in item 14 , the plant or plant tissue as claimed in item 15, the harvested material as claimed in item 15 or the host cell of item as claimed in items 10 or 11.

Item 21. Use of the nuclei acid molecule as defined in item 6, the polypeptide as claimed in item 13, the nucleic acid construct as claimed in item 7, the expression cassette as claimed in item 8, the vector as claimed in item 9, the plant or plant tissue as claimed in item 15, or the host cell of item 10 to 11 for the production of plant resistant to a herbicide inhibiting the production of cerotic acid, heneicosanoic acid, lignoceric acid, melissic acid and/or montanic acid.

Item 22. A method for producing a plant with increased yield as compared to a corresponding non-transformed wild type plant, whereby the method comprises
(i) increasing or generating of the expression of; and/or
(ii) increasing or generating the expression of an expression product of; and/or
(iii) increasing or generating one or more activities of an expression product encoded by; at least one respective nucleic acid molecule comprising a nucleic acid molecule selected from the group consisting of:
(a) a nucleic acid molecule encoding the polypeptide shown in column 5 or 8 of Table II, preferably Table II B, application no. 18, or a homolog or a fragment thereof, in case in column 7 cerotic acid, heneicosanoic acid, lignoceric acid, melissic acid and/or montanic acid is indicated;
(b) a nucleic acid molecule shown in column 5 or 8 of Table I, preferably Table I B, application no. 18, or a homolog or a fragment thereof (preferably the coding region thereof) , in case in column 7 cerotic acid, heneicosanoic acid, lignoceric acid, melissic acid and/or montanic acid is indicated;
(c) a nucleic acid molecule, which, as a result of the degeneracy of the genetic code, can be derived from a polypeptide sequence depicted in column 5 or 8 of Table II, preferably Table II B, application no. 18, in case in column 7 cerotic acid, heneicosanoic acid, lignoceric acid, melissic acid and/or montanic acidis indicated;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 18, or the coding region thereof, in case in column 7 cerotic acid, heneicosanoic acid, lignoceric acid, melissic acid and/or montanic acid is indicated;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 18, in case in column 7 cerotic acid, heneicosanoic acid, lignoceric acid, melissic acid and/or montanic acid is indicated;
(i) a nucleic acid molecule encoding a polypeptide having the activity represented by a respective protein as depicted in column 5 of Table II, application no. 18, in case in column 7 cerotic acid, heneicosanoic acid, lignoceric acid, melissic acid and/or montanic acid is indicated;
(j) a nucleic acid molecule which comprises a polynucleotide, which is obtained by amplifying a cDNA library or a genomic library using the primers as depicted in column 8 of Table III, application no. 18, in case in column 7 cerotic acid, heneicosanoic acid, lignoceric acid, melissic acid and/or montanic acid is indicated; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto; in a plant cell, plant or a part thereof.

Item 23. A method for producing a plant with increased yield according to item 22, which comprises in addition the regeneration of a plant from such transformed plant cell, plant cell compartment, plant cell nucleus, plant cell or plant tissue with increased yield.

[0483.1.7.18] to [0492.1.7.18] for the disclosure of these paragraphs see [0483.1.7.7] to [0492.1.7.7] above

### Example 10

### Purification of a Fine Chemical e.g. VLCFA, e.g. Cerotic Acid, Heneicosanoic Acid, Lignoceric Acid, Melissic Acid and/or Montanic Acid

Long-chain fatty acids, such as cerotic acid, heneicosanoic acid, lignoceric acid, melissic acid and/or montanic acid, can be recovered from cells or from the supernatant of the abovedescribed culture by a variety of methods known in the art. For example, the culture supernatant is recovered first. To this end, the cells are harvested from the culture by slow centrifugation. Cells can generally be disrupted or lysed by standard techniques such as mechanical force or sonication. The cell debris is removed by centrifugation and the supernatant fraction, if appropriate together with the culture supernatant, is used for the further purification of the VLCFA. However, it is also possible to process the supernatant alone if the VLCFA are present in the supernatant in sufficiently high a concentration. In this case, the VLCFA, or the VLCFAs mixture, can be purified further for example via extraction and/or salt precipitation or via ion-exchange chromatography.

One example is the analysis of fatty acids (abbreviations: FAME, fatty acid methyl ester; GC-MS, gas liquid chromatography/mass spectrometry; TAG, triacylglycerol; TLC, thin-layer chromatography).

The unambiguous detection for the presence of fatty acid products can be obtained by analyzing recombinant organisms using analytical standard methods: GC, GC-MS or TLC, as described on several occasions by Christie and the references therein (1997, in: Advances on Lipid Methodology, Fourth Edition: Christie, Oily Press, Dundee, 119-169; 1998, Gaschromatographie-Massenspektrometrie-Verfahren [Gas chromatography/mass spectrometric methods], Lipide 33:343-353).

The total fatty acids produced in the organism for example in yeasts used in the inventive process can be analysed for example according to the following procedure: The material such as yeasts, E. coli or plants to be analyzed can be disrupted by sonication, grinding in a glass mill, liquid nitrogen and grinding or via other applicable methods. After disruption, the material must be centrifuged (1000×g, 10 min., 4° C.) and washed once with 100 mM NaHCO3, pH 8.0 to remove residual medium and fatty acids. For preparation of the fatty acid methyl esters (FAMES) the sediment is resuspended in distilled water, heated for 10 minutes at 100° C., cooled on ice and recentrifuged, followed by extraction for one hour at 90° C. in 0.5 M sulfuric acid in methanol with 2% dimethoxypropane, which leads to hydrolyzed oil and lipid compounds, which give transmethylated lipids.

The FAMES are then extracted twice with 2 ml petrolether, washed once with 100 mM NaHCO₃, pH 8.0 and once with distilled water and dried with Na₂SO₄. The organic solvent can be evaporated under a stream of Argon and the FAMES were dissolved in 50 μl of petrolether. The samples can be separated on a ZEBRON ZB-Wax capillary column (30 m, 0,32 mm, 0.25 μm; Phenomenex) in a Hewlett Packard 6850 gas chromatograph with a flame ionisation detector. The oven temperature is programmed from 70° C. (1 min. hold) to 200° C. at a rate of 20° C/min., then to 250° C. (5 min. hold) at a rate of 5° C/min and finally to 260° C. at a rate of 5° C/min. Nitrogen is used as carrier gas (4,5 ml/min. at 70° C.). The identity of the resulting fatty acid methyl esters can be identified by comparison with retention times of FAME standards, which are available from commercial sources (i.e. Sigma).

Plant material is initially homogenized mechanically by comminuting in a pestle and mortar to make it more amenable to extraction.

This is followed by heating at 100° C. for 10 minutes and, after cooling on ice, by resedimentation. The cell sediment is hydrolyzed for one hour at 90° C. with 1 M methanolic sulfuric acid and 2% dimethoxypropane, and the lipids are transmethylated. The resulting fatty acid methyl esters (FAMEs) are extracted in petroleum ether. The extracted FAMEs are analyzed by gas liquid chromatography using a capillary column (Chrompack, WCOT Fused Silica, CP-Wax-52 CB, 25 m, 0.32 mm) and a temperature gradient of from 170° C. to 240° C. in 20 minutes and 5 minutes at 240° C. The identity of the fatty acid methyl esters is confirmed by comparison with corresponding FAME standards (Sigma). The identity and position of the double bond can be analyzed further by suitable chemical derivatization of the FAME mixtures, for example to give 4,4-dimethoxyoxazoline derivatives (Christie, 1998) by means of GC-MS.

The methodology is described for example in Napier and Michaelson, 2001, Lipids. 36(8):761-766; Sayanova et al., 2001, Journal of Experimental Botany. 52(360):1581-1585, Sperling et al., 2001, Arch. Biochem. Biophys. 388(2):293-298 and Michaelson et al., 1998, FEBS Letters. 439(3):215-218.

If required and desired, further chromatography steps with a suitable resin may follow. Advantageously the fatty acids can be further purified with a so-called RTHPLC. As eluent different an acetonitrile/water or chloroform/acetonitrile mixtures are advantageously is used. For example canola oil can be separated said HPLC method using an RP-18-column (ET 250/3 Nucleosil 120-5 018 Macherey and Nagel, Duren, Germany). A chloroform/acetonitrile mixture (v/v 30:70) is used as eluent. The flow rate is beneficial 0.8 ml/min. For the analysis of the fatty acids an ELSD detector (evaporative light-scattering detector) is used. MPLC, dry-flash chromatography or thin layer chromatography are other beneficial chromatography methods for the purification of VLCFAs. If necessary, these chromatography steps may be repeated, using identical or other chromatography resins. The skilled worker is familiar with the selection of suitable chromatography resin and the most effective use for a particular molecule to be purified. In addition depending on the produced fine chemical purification is also possible with cristalisation or destilation. Both methods are well known to a person skilled in the art.

Identity and purity of the VLCFAs isolated can be determined by standard techniques of the art. They encompass high-performance liquid chromatography (HPLC), spectroscopic methods, mass spectrometry (MS), staining methods, thin-layer chromatography, NIRS, enzyme assay or microbiological assays. These analytical methods are compiled in: Patek et al. (1994) Appl. Environ. Microbiol. 60: 133-140; Malakhova et al. (1996) Biotekhnologiya 11:27-32; and Schmidt et al. (1998) Bioprocess Engineer. 19: 67-70. Ulmann's Encyclopedia of Industrial Chemistry (1996) Vol. A27, VCH: Weinheim, pp. 89-90, pp. 521-540, pp. 540-547, pp. 559-566, 575-581 and pp. 581-587; Michal, G (1999) Biochemical Pathways: An Atlas of Biochemistry and Molecular Biology, John Wiley and Sons; Fallon, A. et al. (1987) Applications of HPLC in Biochemistry in: Laboratory Techniques in Biochemistry and Molecular Biology, Vol. 17.

for the disclosure of this paragraph see [0494.1.7.7] above

### Example 11a

### PCR amplification of the Sequences

Unless otherwise specified, standard methods as described in Sambrook et al., Molecular Cloning: A laboratory manual, Cold Spring Harbor 1989, Cold Spring Harbor Laboratory Press are used.

The inventive sequences as shown in the respective line in Table I, column 5, preferably the coding region thereof, were amplified by PCR as described in the protocol of the Pfu Ultra, Pfu Turbo or Herculase DNA polymerase (Stratagene). The composition for the protocol of the Pfu Ultra, Pfu Turbo or Herculase DNA polymerase was as follows: 1×PCR buffer (Stratagene), 0.2 mM of each dNTP, 100 ng genomic DNA of Saccharomyces cerevisiae (strain S288C; Research Genetics, Inc., now Invitrogen), Escherichia coli (strain MG1655; E. coli Genetic Stock Center), Synechocystis sp. (strain PCC6803), Azotobacter vinelandii (strain N.R. Smith,16), Thermus thermophilus (HB8) or 50 ng cDNA from various tissues and development stages of Arabidopsis thaliana (ecotype Columbia), Physcomitrella patens, Glycine max (variety Resnick), Brassica napus, Oryza sativa or Zea mays (variety B73, Mo17, A188), 50 pmol forward primer, 50 pmol reverse primer, with or without 1 M Betaine, 2.5 u Pfu Ultra, Pfu Turbo or Herculase DNA polymerase.

The amplification cycles were as follows:

1 cycle of 2-3 minutes at 94-95° C., then 25-36 cycles with 30-60 seconds at 94-95° C., 30-45 seconds at 50-60° C. and 210-480 seconds at 72° C., followed by 1 cycle of 5-10 minutes at 72° C., then 4-16° C.—preferably for Saccharomyces cerevisiae, Escherichia coli, Synechocystis sp., Azotobacter vinelandii, Thermus thermophilus.

In case of Arabidopsis thaliana, Brassica napus, Glycine max, Oryza sativa, Physcomitrella patens, Zea mays the amplification cycles were as follows:

1 cycle with 30 seconds at 94° C., 30 seconds at 61° C., 15 minutes at 72° C., then 2 cycles with 30 seconds at 94° C., 30 seconds at 60° C., 15 minutes at 72° C., then 3 cycles with 30 seconds at 94° C., 30 seconds at 59° C., 15 minutes at 72° C., then 4 cycles with 30 seconds at 94° C., 30 seconds at 58° C., 15 minutes at 72° C., then 25 cycles with 30 seconds at 94° C., 30 seconds at 57° C., 15 minutes at 72° C., then 1 cycle with 10 minutes at 72° C., then finally 4-16° C.

RNAs were generated with the RNeasy Plant Kit according to the standard protocol (Qiagen) and Superscript II Reverse Transkriptase was used to produce double stranded cDNA according to the standard protocol (Invitrogen).

ORF specific primer pairs for the genes to be expressed are shown in the respective line in Table III, column 8. The following adapter sequences were added to Saccharomyces cerevisiae ORF specific primers (see Table III) for cloning purposes:

SEQ ID NO: 20

| | | |
|---|---|---|
| i) | foward primer: | 5′-GGAATTCCAGCTGACCACC-3′ |

SEQ ID NO: 21

| | | |
|---|---|---|
| ii) | reverse primer: | 5′-GATCCCCGGGAATTGCCATG-3′ |

These adaptor sequences allow cloning of the ORF into the various vectors containing the Resgen adaptors, see table column E of Table c.

The following adapter sequences were added to Saccharomyces cerevisiae, Escherichia coli, Synechocystis sp., Azotobacter vinelandii, Thermus thermophilus, Arabidopsis thaliana, Brassica napus, Glycine max, Oryza sativa , Physcomitrella patens, or Zea mays ORF specific primers for cloning purposes:

| SEQ ID NO: 22 | | |
|---|---|---|
| iii) | forward primer: | 5′-TTGCTCTTCC-3′ |
| SEQ ID NO: 23 | | |
| iiii) | reverse primer: | 5′-TTGCTCTTCG-3′ |

The adaptor sequences allow cloning of the ORF into the various vectors containing the Colic adaptors, see table column E of Table c.

Therefore for amplification and cloning of Saccharomyces cerevisiae SEQ ID NO: 14843, a primer consisting of the adaptor sequence i) and the ORF specific sequence SEQ ID NO: 14871 and a second primer consisting of the adaptor sequence ii) and the ORF specific sequence SEQ ID NO: 14872 were used.

For amplification and cloning of Escherichia coli SEQ ID NO: 7081, a primer consisting of the adaptor sequence iii) and the ORF specific sequence SEQ ID NO: 7261 and a second primer consisting of the adaptor sequence iiii) and the ORF specific sequence SEQ ID NO: 7262 were used.

For amplification and cloning of Synechocystis sp. SEQ ID NO: 47526, a primer consisting of the adaptor sequence iii) and the ORF specific sequence SEQ ID NO: 47562 and a second primer consisting of the adaptor sequence iiii) and the ORF specific sequence SEQ ID NO: 47563 were used.

For amplification and cloning of Arabidopsis thaliana SEQ ID NO: 2935, a primer consisting of the adaptor sequence iii) and the ORF specific sequence SEQ ID NO: 3275 and a second primer consisting of the adaptor sequence iiii) and the ORF specific sequence SEQ ID NO: 3276 were used.

For amplification and cloning of Azotobacter vinelandii SEQ ID NO: 6510, a primer consisting of the adaptor sequence iii) and the ORF specific sequence SEQ ID NO: 6670 and a second primer consisting of the adaptor sequence iiii) and the ORF specific sequence SEQ ID NO: 6671 were used.

For amplification and cloning of Glycine max SEQ ID NO: 10811, a primer consisting of the adaptor sequence iii) and the ORF specific sequence SEQ ID NO: 11207 and a second primer consisting of the adaptor sequence iiii) and the ORF specific sequence SEQ ID NO: 11208 were used.

For amplification and cloning of Thermus thermophilus SEQ ID NO: 12698, a primer consisting of the adaptor sequence iii) and the ORF specific sequence SEQ ID NO: 12970 and a second primer consisting of the adaptor sequence iiii) and the ORF specific sequence SEQ ID NO: 12971 were used.

Following these examples every sequence disclosed in Table I, preferably column 5, especially the coding region thereof can be cloned by fusing the adaptor sequences to the respective specific primers sequences as disclosed in Table III, column 8 using the respective vectors shown in Table c.

[0496.1.7.18] to [0499.1.7.18] for the disclosure of these paragraphs see [0496.1.7.7] to [0499.1.7.7] above

### Example 11e

### Cloning of Inventive Sequences as Shown in Table I, Column 5 in the Different Expression Vectors

For cloning of for example SEQ ID NO: 7081 from Escherichia coli or any other ORF from Escherichia coli, Synechocystis sp., Azotobacter vinelandii, Thermus thermophilus, Arabidopsis thaliana, or Glycine max the vector DNA was treated with the restriction enzymes Pacl and Ncol following the standard protocol (MBI Fermentas).

For cloning for example the ORFs of SEQ ID NO: 14843 from Saccharomyces cerevisiae or any other ORF from Saccharomyces cerevisiae into vectors containing the Resgen adaptor sequence the respective vector DNA was treated with the restriction enzyme Ncol.

For cloning of for example ORFs SEQ ID NO: 63665 from Saccharomyces cerevisiae or any other ORF from Saccharomyces cerevisiae into vectors containing the Colic adaptor sequence, the respective vector DNA was treated with the restriction enzymes Pacl and Ncol following the standard protocol (MBI Fermentas).

In all cases the reaction was stopped by inactivation at 70° C. for 20 minutes and purified over QlAquick or NucleoSpin Extract II columns following the standard protocol (Qiagen or Macherey-Nagel).

Then the PCR-product representing the amplified ORF with the respective adapter sequences and the vector DNA were treated with T4 DNA polymerase according to the standard protocol (MBI Fermentas) to produce single stranded overhangs with the parameters 1 unit T4 DNA polymerase at 37° C. for 2-10 minutes for the vector and 1-2 u T4 DNA polymerase at 15-17° C. for 10-60 minutes for the PCR product representing SEQ ID NO: 7081.

The reaction was stopped by addition of high-salt buffer and purified over QlAquick or NucleoSpin Extract II columns following the standard protocol (Qiagen or Macherey-Nagel).

According to this example the skilled person is able to clone all sequences disclosed in Table I, preferably column 5 or column 8, especially the coding region thereof.

Approximately 30-60 ng of prepared vector and a defined amount of prepared amplificate were mixed and hybridized at 65° C. for 15 minutes followed by 37° C. 0.1° C/1 seconds, followed by 37° C. 10 minutes, followed by 0.1° C/1 seconds, then 4-10 ° C.

The ligated constructs were transformed in the same reaction vessel by addition of competent E. coli cells (strain DHSalpha) and incubation for 20 minutes at 1° C. followed by a heat shock for 90 seconds at 42° C. and cooling to 1-4° C. Then, complete medium (SOC) was added and the mixture was incubated for 45 minutes at 37° C. The entire mixture was subsequently plated onto an agar plate with 0.05 mg/ml kanamycin and incubated overnight at 37° C.

The outcome of the cloning step was verified by amplification with the aid of primers which bind upstream and downstream of the integration site, thus allowing the amplification of the insertion. The amplifications were carried out as described in the protocol of Taq DNA polymerase (Gibco-BRL).

The amplification cycles were as follows:

1 cycle of 1-5 minutes at 94° C., followed by 35 cycles of in each case 15-60 seconds at 94° C., 15-60 seconds at 50-66° C. and 5-15 minutes at 72° C., followed by 1 cycle of 10 minutes at 72° C., then 4-16° C.

Several colonies were checked, but only one colony for which a PCR product of the expected size was detected was used in the following steps.

A portion of this positive colony was transferred into a reaction vessel filled with complete medium (LB) supplemented with kanamycin and incubated overnight at 37° C.

The plasmid preparation was carried out as specified in the Qiaprep or NucleoSpin Multi-96 Plus standard protocol (Qiagen or Macherey-Nagel).

[0501.1.7.18] to [0503.1.7.18] for the disclosure of these paragraphs see [0501.1.7.7] to

Table d showing results of plant analyses

TABLE d

| SeqID | Target | Locus | Metabolite | Source | Promotor | Method | Min | Max |
|---|---|---|---|---|---|---|---|---|
| 17968 | non-targ | At1g36730 | lignoceric acid | ARA_LEAF | p-PcUBI | GC | 31 | 65 |
| 18235 | non-targ | At1g48260 | lignoceric acid | ARA_LEAF | p-PcUBI | GC | 33 | 86 |
| 22249 | non-targ | At3g08710 | lignoceric acid | ARA_LEAF | p-PcUBI | GC | 31 | 127 |
| 152081 | non-targ | At3g09940 | lignoceric acid | ARA_LEAF | p-PcUBI | GC | 28 | 36 |
| 68777 | non-targ | At3g27540 | lignoceric acid | ARA_LEAF | p-PcUBI | GC | 39 | 99 |
| 2935 | non-targ | At3g62950 | lignoceric acid | ARA_LEAF | p-PcUBI | GC | 30 | 159 |
| 24232 | non-targ | At4g18880 | lignoceric acid | ARA_LEAF | p-PcUBI | GC | 27 | 53 |
| 4348 | non-targ | At4g35310 | lignoceric acid | ARA_LEAF | p-PcUBI | GC | 38 | 213 |
| 24492 | non-targ | At5g07200 | lignoceric acid | ARA_LEAF | p-PcUBI | GC | 28 | 50 |
| 5493 | non-targ | At5g64920 | lignoceric acid | ARA_LEAF | p-PcUBI | GC | 30 | 57 |
| 31926 | non-targ | Avin- | lignoceric acid | ARA_LEAF | p-PcUBI | GC | 31 | 40 |
| | | DRAFT_4562 | | | | | | |
| 6510 | non-targ | Avin- | lignoceric acid | ARA_LEAF | p-PcUBI | GC | 35 | 171 |
| | | DRAFT_5103 | | | | | | |
| 32037 | non-targ | Avin- | lignoceric acid | ARA_LEAF | p-PcUBI | GC | 27 | 137 |
| | | DRAFT_5246 | | | | | | |
| 33457 | non-targ | Avin- | lignoceric acid | ARA_LEAF | p-PcUBI | GC | 36 | 62 |
| | | DRAFT_5651 | | | | | | |
| 33596 | non-targ | Avin- | lignoceric acid | ARA_LEAF | p-PcUBI | GC | 32 | 50 |
| | | DRAFT_6093 | | | | | | |
| 34044 | non-targ | Avin- | lignoceric acid | ARA_LEAF | p-PcUBI | GC | 27 | 99 |
| | | DRAFT_6700 | | | | | | |
| 34889 | plastidic | B0004 | lignoceric acid | ARA_LEAF | p-Super | GC | 32 | 201 |
| 7081 | non-targ | B0161 | lignoceric acid | ARA_LEAF | p-Super | GC | 43 | 268 |
| 7333 | non-targ | B0449 | lignoceric acid | ARA_LEAF | p-Super | GC | 34 | 244 |
| 35967 | plastidic | B0593 | lignoceric acid | ARA_LEAF | p-Super | GC | 30 | 87 |
| 36489 | non-targ | B0885 | lignoceric acid | ARA_LEAF | p-Super | GC | 38 | 93 |
| 7917 | non-targ | B0898 | lignoceric acid | ARA_LEAF | p-Super | GC | 36 | 337 |
| 76032 | plastidic | B1065 | cerotic acid | ARA_LEAF | p-Super | GC | 41 | 48 |
| 37400 | non-targ | B1186 | lignoceric acid | ARA_LEAF | p-Super | GC | 35 | 126 |
| 98778 | non-targ | B1249 | lignoceric acid | ARA_LEAF | p-Super | GC | 33 | 73 |
| 7947 | non-targ | B1522 | lignoceric acid | ARA_LEAF | p-Super | GC | 28 | 178 |
| 38300 | non-targ | B1597 | cerotic acid | ARA_LEAF | p-Super | GC | 43 | 164 |
| 38300 | non-targ | B1597 | lignoceric acid | ARA_LEAF | p-Super | GC | 35 | 214 |
| 79217 | non-targ | B2027 | lignoceric acid | ARA_LEAF | p-Super | GC | 29 | 85 |
| 79509 | non-targ | B2482 | lignoceric acid | ARA_LEAF | p-Super | GC | 29 | 64 |
| 40383 | non-targ | B2541 | lignoceric acid | ARA_LEAF | p-Super | GC | 40 | 110 |
| 82424 | non-targ | B3083 | lignoceric acid | ARA_LEAF | p-Super | GC | 31 | 63 |
| 125487 | non-targ | B3812 | lignoceric acid | ARA_LEAF | p-Super | GC | 35 | 118 |
| 10740 | non-targ | B4029 | lignoceric acid | ARA_LEAF | p-Super | GC | 31 | 222 |
| 45321 | non-targ | B4256 | lignoceric acid | ARA_LEAF | p-Super | GC | 64 | 185 |
| 10811 | non-targ | GM02LC12622 | lignoceric acid | ARA_LEAF | p-PcUBI | GC | 28 | 81 |
| 46850 | non-targ | GM02LC44512 | lignoceric acid | ARA_LEAF | p-PcUBI | GC | 30 | 75 |
| 47526 | mito- | SII0064 | lignoceric acid | ARA_LEAF | p-PcUBI | GC | 29 | 56 |
| | chon- | | | | | | | |
| | drial | | | | | | | |
| 84265 | plastidic | SII0228 | lignoceric acid | ARA_LEAF | p-PcUBI | GC | 36 | 38 |
| 84869 | plastidic | SII0378 | lignoceric acid | ARA_LEAF | p-PcUBI | GC | 32 | 69 |
| 101707 | non-targ | SII0833 | lignoceric acid | ARA_LEAF | p-PcUBI | GC | 40 | 112 |
| 51268 | plastidic | SII0891 | lignoceric acid | ARA_LEAF | p-PcUBI | GC | 32 | 55 |
| 85743 | mito- | SII1598 | lignoceric acid | ARA_LEAF | p-PcUBI | GC | 27 | 51 |
| | chon- | | | | | | | |
| | drial | | | | | | | |
| 86447 | plastidic | SII1848 | lignoceric acid | ARA_LEAF | p-PcUBI | GC | 57 | 85 |
| 95744 | non-targ | SIr0383 | lignoceric acid | ARA_LEAF | p-PcUBI | GC | 32 | 48 |
| 57235 | plastidic | SIr0739 | lignoceric acid | ARA_LEAF | p-PcUBI | GC | 31 | 87 |
| 87655 | plastidic | SIr0966 | lignoceric acid | ARA_LEAF | p-PcUBI | GC | 32 | 111 |
| 152275 | non-targ | SIr2033 | lignoceric acid | ARA_LEAF | p-PcUBI | GC | 28 | 105 |
| 12698 | non-targ | TTC0019 | lignoceric acid | ARA_LEAF | p-PcUBI | GC | 44 | 114 |
| 61532 | non-targ | TTC0768 | lignoceric acid | ARA_LEAF | p-PcUBI | GC | 38 | 110 |
| 62079 | non-targ | TTC1386 | lignoceric acid | ARA_LEAF | p-PcUBI | GC | 49 | 106 |
| 121908 | non-targ | YaI054c | lignoceric acid | ARA_LEAF | p-PcUBI | GC | 52 | 58 |
| 63665 | plastidic | Ydr265w | lignoceric acid | ARA_LEAF | p-PcUBI | GC | 28 | 50 |
| 149632 | non-targ | Yjl153c | heneicosanoic | ARA_SEED_2 | p-PcUBI | GC | 10 | 23 |
| | | | acid | | | | | |
| 14843 | non-targ | Yjr130c | lignoceric acid | ARA_LEAF | Big35S | GC | 31 | 42 |
| 152382 | non-targ | Ykr050w | lignoceric acid | ARA_LEAF | Big35S | GC | 28 | 96 |
| 66695 | plastidic | YIr065c | lignoceric acid | ARA_LEAF | p-Super | GC | 28 | 66 |
| 152424 | plastidic | YmI093w | lignoceric acid | ARA_LEAF | p-Super | GC | 34 | 69 |
| 92274 | plastidic | Ymr150c | lignoceric acid | ARA_LEAF | p-Super | GC | 29 | 74 |
| 152460 | plastidic | Ymr208w | lignoceric acid | ARA_LEAF | p-Super | GC | 30 | 44 |
| 152341 | non-targ | Yer152c | melissic acid | ARA_LEAF | Big35S | GC | 1 | 69 |
| 152341 | non-targ | Yer152c | montanic acid | ARA_LEAF | Big35S | GC | 3 | 93 |
| 125348 | non-targ | B3644 | melissic acid | ARA_LEAF | p-Super | GC | 3 | 56 |
| 125348 | non-targ | B3644 | montanic acid | ARA_LEAF | p-Super | GC | 5 | 49 |

Column 1 shows the SEQ ID NO, Column 2 shows the expression type (targeted or non-targeted), Column 3 shows the “gene name” (locus), Column 4 shows the metabolite analyzed, Column 5 indicates the A. thaliana source tissue analyzed, Column 6 indicates the used promoter for expression, Column 7 indicates the analytical method. Columns 8 and 9 show the minimum and the maximum increase of the analyzed metabolite (in percent) in comparison to the wild type (ratio_by _WT, given as percent increase).

The term “non-targ” in Table d, Column 2 which shows the expression type means “non-targeted”.

When the analyses were repeated independently, all results proved to be significant.

[0505.1.7.18] to [0515.1.7.18] for the disclosure of these paragraphs see [0505.1.7.7] to [0515.1.7.7] above

In a further embodiment, the present invention relates in paragraphs [0000.1.7.19.] to [0514.1.7.19.] to a further process for the production of the fine chemical bis-glyceryl phosphate (polar fraction), glycerol (lipid fraction), glycerol (polar fraction), glycerol phosphate (lipid fraction), glycerol-3-phosphate (polar fraction), and/or triacylglycerol acid as defined below and corresponding embodiments as described herein as follows.

for the disclosure of this paragraph see [0001.1.7.7] above

Plants produce glycerol and glycerol-3-phosphate. Importantly lipids derived from glycerol are the major components of eukaryotic cells. In terms of dry weight they account for anywhere between 10% and 90% of the total mass of the cell. Triglycerol are the major source of store energy in eucaryotic organisms.

Glycerol is a principal component of all fats and oils, in the form of its esters called glycerides. It is commercially known as glycerin or glycerine. Glycerol (CH2OH.CHOH.CH2OH or propane-1,2,3-triol), in its pure form, is a sweet-tasting, clear, colorless, odorless, viscous liquid. It is completely soluble in water and alcohols, slightly soluble in many common solvents such as ether and dioxane, and is insoluble in hydrocarbons. As a food additive, glycerol is labeled as E number E422.

Glycerol is widely distributed in all living organisms as a constituent of glycerides. Glycerol is a precursor for synthesis of triacylglycerols and of phospholipids in the liver and adipose tissue. During digestion, glycerol is split from fatty acids and may recombine with them to form stored fat in the body or be used as a body fuel to provide energy. Before glycerol can enter the pathway of glycolysis or gluconeogenesis, it must be converted to their intermediate glyceraldehyde 3-phosphate.

Glycerol-3-phosphate (G3P, G-3-P) can be synthesized via two different routes in plants. In one route, it is formed from dihydroxyacetone phosphate (DHAP), an intermediate of glycolysis. In the second pathway it is formed by the reduction of dihydroxyacetone phosphate (DHAP).

In plants, at least two types of GPDH, a cytoplasmatic and a plastidial exist, which also differ in their reducing cosubstrate. The cytsolic GPDH uses NADH as the cosubstrate. The mitochondrial FAD-dependent glycerol-3-phosphate dehydrogenase (FAD-GPDH) of Arabidopsis forms a G-3-P shuttle, as previously established in other eukaryotic organisms, and links cytosolic G-3-P metabolism to carbon source utilization and energy metabolism in plants—also see Shen, W. et al., FEBS Lett. 2003 Feb 11; 536(1-3): 92-6.

Glycerol-3-phosphate is a primary substrate for triacylglycerol synthesis. Vigeolas and Geigenberger (Planta 219(2004): 827-835) have shown that injection of developing seeds with glycerol leads to increased glycerol-3-phosphate levels. These increased levels of glycerol-3-phosphate were accompanied by an increase in the flux of sucrose into total lipids and triacylglycerol providing evidence that the prevailing levels of glycerol-3-phosphate co-limit triacylglycerol production in developing seeds.

The direct acylation of glycerol by a glycerol: acyl-CoA acyltransferase to form mono-acyl-glycerol and, subsequently, diacylglycerol and triacylglycerol has been shown in myoblast and hepatocytes (Lee, D. P. et al. J. Lipid res. 42 (2001): 1979-1986). This direct acylation became more prominent when the glycerol-3-phosphate pathway was attenuated or when glycerol levels become elevated.

The major storage lipids (or oils) of seeds occur in the form of triacylglycerols (TAG), or three fatty acids linked to glycerol by ester bonds. Triacylglycerol synthesis involves diverse cellular compartments, including the cytoplasm, the mitochondria, the plastids, and the endoplasmic reticulum (ER). Glycerol-3-phosphate enters the ER for the final step in triacylglycerol synthesis. The newly formed triacylglycerols accumulate between the two layers of the double membrane of the ER, forming an oil body surrounded by a single (or half) unit membrane. Bis-glyceryl phosphate (glycerophophoryl glycerol, GPG) is a glycerophosphodiester which is formed by deacylation of phospholipids.

Glycerol was found to possess a wide variety of uses in the manufacture of numerous domestic, industrial, and pharmaceutical products.

Glycerol-insensitive Arabidopsis mutants: glil seedlings lack glycerol kinase, accumulate glycerol and are more resistant to abiotic stress, see Eastmond P.J. , The Plant Journal, 2004, 37(4), 617-625. These data show that glycerol kinase is required for glycerol catabolism in Arabidopsis and that the accumulation of glycerol can enhance resistance to a variety of abiotic stresses associated with dehydration.

Glycerol is used together with water and alcohol (ethyl alcohol) in glycerinated water/alcohol plant extracts and phytoaromatic compounds. These products are used as food supplements, providing concentrates of the minerals, trace elements, active ingredients (alkaloids, polyphenols, pigments, etc.) and aromatic substances to be found in plants. Glycerin acts as a carrier for plant extracts. It is found in the end product (the fresh plant extract) in concentrations of up to 24% or 25%.

Raw glycerol is a by-product of the transesterification process of rape oil to rape methyl ester (RME) and used edible oil to used edible methyl ester (AME), both better known as Biodiesel. Glycerol world production is estimated to be around 750.000 t/year. Around 90% is manufactured on the basis of natural oils and fats.

Glycerol or glycerol-phosphate, preferably G3P are biosynthetic precusors for the biosynthesis of monoacylglycerols, diacylglycerols, triacylglycerols, phosphatidylglycerols and other glycerolipids (e.g. glycosylglycerides, diphosphatidylglycerols, phosphonolipids, phosphatidylcholines, phosphatidylethanolamines, phosphatidylinositols, phytoglycolipids). Therefore the analysis of the glycerol or glycerol-phosphate content in cells, tissues or plant parts like seeds and leaves after total lipid extraction and lipid hydrolysis directly correlates with the analysis of the total lipid content. For example if the overexpression of a gene participating in the biosynthesis of triacylglycerols in the seed results in an increase in total lipid content in the seed or leaf this seed will also show an increased glycerol content after total lipid extraction and hydrolysis of the lipids.

Therefore the method as described below which leads to an increase in glycerol or glycerolphosphate in the lipid fraction after cleavage of the ester functions for example with a mixture of methanol and hydrochloric acid clearly represents a method for an increased production of triacylglycerol or total lipids.

Due to the wide variety of uses of glycerol and/or its derivates like glycerolphosphate, preferably glycerol-3-phosphate and/or GPG and/or monoacylglycerols, diacylglycerols, triacylglycerols, phosphatidylglycerols and/or other glycerolipids an increased production very important.

Particularly economical methods are biotechnological methods utilizing glycerol-producing organisms which are either natural or optimized by genetic modification.

There is a constant need for providing novel enzyme activities or direct or indirect regulators and thus alternative methods with advantageous properties for producing glycerol or its derivates in organisms, e.g. in transgenic organi sms.

The content of glycerol in a cell was also increased by increasing or decreasing certain activities as disclosed in WO 2006/069610, WO2007/087815 or WO2008/034648

Improving the quality of foodstuffs and animal feeds is an important task of the food-and-feed industry. This is necessary since, for example, glycerol and/or its derivates, which occur in plants are limited with regard to the supply of mammals. Especially advantageous for the quality of foodstuffs and animal feeds is as balanced as possible a glycerol and/or lipidprofile since a great excess of some lipids above a specific concentration in the food has no further positive effect on the utilization of the food since other lipids suddenly become limiting. A further increase in quality is only possible via addition of further lipids, which are limiting under these conditions. The targeted addition of the limiting lipid in the form of synthetic products must be carried out with extreme caution in order to avoid lipid imbalance.

To ensure a high quality of foods and animal feeds, it is therefore necessary to add a plurality of lipids in a balanced manner to suit the respective organism. Accordingly, there is still a great demand for new and more suitable genes, which encode enzymes or regulators, which participate in the biosynthesis of lipids, in particular glycerol and/or its derivates and make it possible to produce certain lipids specifically on an industrial scale without unwanted byproducts being formed. In the selection of genes for biosynthesis or regulation two characteristics above all are particularly important. On the one hand, there is as ever a need for improved processes for obtaining the highest possible contents of lipids like glycerol and/or its derivates and on the other hand as less as possible byproducts should be produced in the production process.

It is an object of the present invention to develop an inexpensive process for the synthesis of bis-glyceryl phosphate (polar fraction), glycerol (lipid fraction), glycerol (polar fraction), glycerol phosphate (lipid fraction), glycerol-3-phosphate (polar fraction), and/or triacylglycerol.

It is a further object of the present invention to develop an inexpensive process for the synthesis of lipids and/or glycerol and derivates, in particular bis-glyceryl phosphate (polar fraction), glycerol (lipid fraction), glycerol (polar fraction), glycerol phosphate (lipid fraction), glycerol-3-phosphate (polar fraction), and/or triacylglycerol and to assure that the lipids and/or glycerol and derivates, in particular bis-glyceryl phosphate (polar fraction), glycerol (lipid fraction), glycerol (polar fraction), glycerol phosphate (lipid fraction), glycerol-3-phosphate (polar fraction), and/or triacylglycerol are more accessible and facilely to isolate and recover in an industrial scale from the producing organism, preferably from a plant.

for the disclosure of this paragraph see [0013.1.7.7] above

Accordingly, in a first embodiment, the invention relates to a process for the production of at least one fine chemical selected from the group consisting of: bis-glyceryl phosphate (polar fraction), glycerol (lipid fraction), glycerol (polar fraction), glycerol phosphate (lipid fraction), glycerol-3-phosphate (polar fraction), and triacylglycerol, or, in other words, of the “fine chemical” or “fine chemical of the invention”.

The terms “fine chemical of the invention”, “fine chemical” or “the fine chemical” are used herein equally and relate in context of the paragraphs or sections [0014.1.19.19] to [0514.1.7.19] essentially to the metabolite or the metabolites indicated in column 7, application no. 19 of Tables I, II and IV in the respective line.

Further, the term “in context of any of the paragraphs [0014.1.19.19] to [0514.1.7.19]” as used herein means that for any of said paragraphs [0014.1.19.19] to [0514.1.7.19] the term “the fine chemical” is understood to follow the definition of paragraphs or sections [0014.1.19.19] and [0015.1.19.19], independently whether it refers to any other paragraph or not and whether the reference recites the term “fine chemical” in an other context.

Thus, in cases where one or more paragraphs or sections are incorporated by reference into any of the present paragraphs [0014.1.19.19] to [0514.1.7.19], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to the definition of paragraph [0014.1.19.19] and [0015.1.19.19].

Accordingly, in one embodiment, the term “fine chemical of the invention”, “fine chemical” or “the fine chemical” as used herein (alone or in combination, like in FCRP) means “bis-glyceryl phosphate (polar fraction)” in context of the nucleic acid or polypeptide sequences listed in the respective same line of any one of Tables Ito IV of application no. 19 and indicating in column 7 the metabolite “bis-glyceryl phosphate (polar fraction)”.

In one embodiment, the term bis-glyceryl phosphate (polar fraction) or the term “fine chemical” mean in context of the paragraphs or sections [0014.1.19.19] to [0514.1.7.19] at least one chemical compound with an activity of the above mentioned bis-glyceryl phosphate (polar fraction), respectively.

Accordingly, in one embodiment, the term “fine chemical of the invention”, “fine chemical” or “the fine chemical” as used herein (alone or in combination, like in FCRP) means “glycerol (lipid fraction)” in context of the nucleic acid or polypeptide sequences listed in the respective same line of any one of Tables I to IV of application no. 19 and indicating in column 7 the metabolite “glycerol (lipid fraction)”.

In one embodiment, the term glycerol (lipid fraction) or the term “fine chemical” mean in context of the paragraphs or sections [0014.1.19.19] to [0514.1.7.19] at least one chemical compound with an activity of the above mentioned glycerol (lipid fraction), respectively.

Accordingly, in one embodiment, the term “fine chemical of the invention”, “fine chemical” or “the fine chemical” as used herein (alone or in combination, like in FCRP) means “glycerol (polar fraction)” in context of the nucleic acid or polypeptide sequences listed in the respective same line of any one of Tables Ito IV of application no. 19 and indicating in column 7 the metabolite “glycerol (polar fraction)”.

In one embodiment, the term glycerol (polar fraction) or the term “fine chemical” mean in context of the paragraphs or sections [0014.1.19.19] to [0514.1.7.19] at least one chemical compound with an activity of the above mentioned glycerol (polar fraction), respectively.

Accordingly, in one embodiment, the term “fine chemical of the invention”, “fine chemical” or “the fine chemical” as used herein (alone or in combination, like in FCRP) means “glycerol phosphate (lipid fraction)” in context of the nucleic acid or polypeptide sequences listed in the respective same line of any one of Tables Ito IV of application no. 19 and indicating in column 7 the metabolite “glycerol phosphate (lipid fraction)”.

In one embodiment, the term glycerol phosphate (lipid fraction) or the term “fine chemical” mean in context of the paragraphs or sections [0014.1.19.19] to [0514.1.7.19] at least one chemical compound with an activity of the above mentioned glycerol phosphate (lipid fraction), respectively.

Accordingly, in one embodiment, the term “fine chemical of the invention”, “fine chemical” or “the fine chemical” as used herein (alone or in combination, like in FCRP) means “glycerol-3-phosphate (polar fraction)” in context of the nucleic acid or polypeptide sequences listed in the respective same line of any one of Tables I to IV of application no. 19 and indicating in column 7 the metabolite “glycerol-3-phosphate (polar fraction)”.

In one embodiment, the term glycerol-3-phosphate (polar fraction) or the term “fine chemical” mean in context of the paragraphs or sections [0014.1.19.19] to [0514.1.7.19] at least one chemical compound with an activity of the above mentioned glycerol-3-phosphate (polar fraction), respectively.

Accordingly, in one embodiment, the term “fine chemical of the invention”, “fine chemical” or “the fine chemical” as used herein (alone or in combination, like in FCRP) means “triacylglycerols” in context of the nucleic acid or polypeptide sequences listed in the respective same line of any one of Tables I to IV of application no. 19 and indicating in column 7 the metabolite “triacylglycerol”.

In one embodiment, the term triacylglycerol or the term “fine chemical” mean in context of the paragraphs or sections [0014.1.19.19] to [0514.1.7.19] at least one chemical compound with an activity of the above mentioned triacylglycerol, respectively.

Accordingly, in one embodiment, the terms “fine chemical of the invention”, “fine chemical” or “the fine chemical” mean in context of any of the paragraphs [0014.1.19.19] to [0514.1.7.19] bis-glyceryl phosphate (polar fraction), its salts, ester or ether in free form or bound form. In a preferred embodiment, the term “the fine chemical” means bis-glyceryl phosphate (polar fraction) or its salts, ester or ether, in free form or bound form.

In a preferred embodiment “bis-glyceryl phosphate (polar fraction)” means bis-glyceryl phosphate (polar fraction) in free form.

Accordingly, in one embodiment, the terms “fine chemical of the invention”, “fine chemical” or “the fine chemical” mean in context of any of the paragraphs [0014.1.19.19] to [0514.1.7.19] glycerol (lipid fraction), its salts, ester or ether in free form or bound form. In a preferred embodiment, the term “the fine chemical” means glycerol (lipid fraction) or its salts, ester or ether, in free form or bound form.

In a preferred embodiment “glycerol (lipid fraction)” means glycerol (lipid fraction) in free form.

Accordingly, in one embodiment, the terms “fine chemical of the invention”, “fine chemical” or “the fine chemical” mean in context of any of the paragraphs [0014.1.19.19] to [0514.1.7.19] glycerol (polar fraction), its salts, ester or ether in free form or bound form. In a preferred embodiment, the term “the fine chemical” means glycerol (polar fraction) or its salts, ester or ether, in free form or bound form.

In a preferred embodiment “glycerol (polar fraction)” means glycerol (polar fraction) in free form.

Accordingly, in one embodiment, the terms “fine chemical of the invention”, “fine chemical” or “the fine chemical” mean in context of any of the paragraphs [0014.1.19.19] to [0514.1.7.19] glycerol phosphate (lipid fraction), its salts, ester or ether in free form or bound form. In a preferred embodiment, the term “the fine chemical” means glycerol phosphate (lipid fraction) or its salts, ester or ether, in free form or bound form.

In a preferred embodiment “glycerol phosphate (lipid fraction)” means glycerol phosphate (lipid fraction) in free form.

Accordingly, in one embodiment, the terms “fine chemical of the invention”, “fine chemical” or “the fine chemical” mean in context of any of the paragraphs [0014.1.19.19] to [0514.1.7.19] glycerol-3-phosphate (polar fraction), its salts, ester or ether in free form or bound form. In a preferred embodiment, the term “the fine chemical” means glycerol-3-phosphate (polar fraction) or its salts, ester or ether, in free form or bound form.

In a preferred embodiment “glycerol-3-phosphate (polar fraction)” means glycerol-3-phosphate (polar fraction) in free form.

Accordingly, in one embodiment, the terms “fine chemical of the invention”, “fine chemical” or “the fine chemical” mean in context of any of the paragraphs [0014.1.19.19] to [0514.1.7.19] triacylglycerol, its salts, ester or ether in free form or bound form. In a preferred embodiment, the term “the fine chemical” means triacylglycerol or its salts, ester or ether, in free form or bound form.

In a preferred embodiment “triacylglycerol” means triacylglycerol in free form.

Accordingly, in a first embodiment, the invention relates to a process for the production of at least one fine chemical selected from the group consisting of: bis-glyceryl phosphate (polar fraction), glycerol (lipid fraction), glycerol (polar fraction), glycerol phosphate (lipid fraction), glycerol-3-phosphate (polar fraction) and/or triacylglycerol, or, in other words, of the “fine chemical” or “fine chemical of the invention”. Accordingly, in the present invention, the term “the fine chemical” as used herein relates to “glycerol and/or glycerolphosphate and/or glycerophosphodiester, preferably G3P and/or GPG and/or derivates”. Further, the term “the fine chemicals” as used herein also relates to fine chemicals comprising glycerol and/or glycerolphosphate and/or glycerolphosphate and/or glycerophosphodiester,and/or glycerides, lipids, oils and/or fats containing glycerol. Further, the term “the fine chemicals” as used herein also relates to fine chemicals comprising glycerol.

Further, the term “the fine chemicals” as used herein also relates to derivates of glycerol and/or glycerolphosphate and/or glycerophosphodiester, preferably G3P and/or GPG resp. like salts, ester, ethers, thioesters, thioethers mixtures thereof in free form or bound to other compounds such as protein(s) such as enzyme(s), peptide(s), polypeptide(s), membranes or part thereof, or lipids, oils, waxes or fatty acids or mixtures thereof or in compositions with lipids or carbohydrates such as sugars or sugarpolymers, like glucosides or polyols like myo-inositol or mixtures thereof.

In one embodiment, the term “the fine chemical” means monoacylglycerols, diacylglycerols, triacylglycerols, phosphatidylglycerols and/or other glycerolipids (e.g. but not limited to glycosylglycerides, diphosphatidylglycerols, phosphonolipids, phosphatidylcholines, phosphatidylethanolamines, phosphatidylinositols or phytoglycolipids) and is hereinafter referred to as “total lipids”.

In one embodiment, the term “glycerol” or “the fine chemical” or “the respective fine chemical” means at least one chemical compound with glycerol activity selected from the group glycerol and/or glycerolphosphate and/or glycerophosphodiester, preferably G3P and/or GPG resp. and/or derivates selected from the group of monoacylglycerols, diacylglycerols, triacylglycerols, phosphatidylglycerols and/or other glycerolipids (e.g. but not limited to glycosylglycerides, diphosphatidylglycerols, phosphonolipids, phosphatidylcholines, phosphatidylethanolamines, phosphatidylinositols or phytoglycolipids). In another embodiment, the term “glycerol” or “the fine chemical” or “the respective fine chemical” means at least one chemical compound with glycerol activity selected from the group glycerol and/or glycerolphosphate, preferably glycerol-3-phosphate and/or monoacylglycerols, diacylglycerols, triacylglycerols, phosphatidylglycerols and/or other glycerolipids (e.g. but not limited to glycosylglycerides, diphosphatidylglycerols, phosphonolipids, phosphatidylcholines, phosphatidylethanolamines, phosphatidylinositols or phytoglycolipids) in free or bound form.

In one embodiment derivates of glycerol means according to the present invention glycerolphosphate and/or glycerophosphodiester, preferably G3P and/or GPG resp.; salts, ester, ethers, thioesters, thioethers mixtures thereof or monoacylglycerols, diacylglycerols, triacylglycerols, preferably triacylglycerols, phosphatidylglycerols and/or other glycerolipids (e.g. but not limited to glycosylglycerides, diphosphatidylglycerols, phosphonolipids, phosphatidylcholines, phosphatidylethanolamines, phosphatidylinositols or phytoglycolipids) and lipids.

Further, the term “in context of any of the paragraphs [0014.1.19.19] to [0514.1.7.19]” as used herein means that for any of said paragraphs [0014.1.19.19] to [0514.1.7.19] the term “the fine chemical” is understood to follow the definition of section [0014.1.19.19] or section [0015.1.19.19], independently whether it refers to any other paragraph or not and whether the reference recites the term “fine chemical” in an other context.

Thus, in cases where one or more paragraphs or sections are incorporated by reference into any of the present paragraphs [0014.1.19.19] to [0514.1.7.19], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to this definition of this paragraph [0015.1.19.19].

Further, the term “fine chemicals” as used herein relates to compositions comprising said fine chemical(s), i.e. comprising bis-glyceryl phosphate (polar fraction), glycerol (lipid fraction), glycerol (polar fraction), glycerol phosphate (lipid fraction), glycerol-3-phosphate (polar fraction), and/or triacylglycerol, respectively.

As glycerol or glycerolphosphate, preferably G3P are biosynthetic precusor for the biosynthesis of monoacylglycerols, diacylglycerols, triacylglycerols, phosphatidylglycerols and other glycerolipids (e.g. g lycosylg lycerid es, diphosphatidylglycerols, phosphonolipids, phosphatidylcholines, phosphatidylethanolamines, phosphatidylinositols, phytoglycolipids). Therefore the analysis of the glycerol content in cells, tissues or plant parts like seeds and leaves after total lipid extraction and lipid hydrolysis directly correlates with the analysis of the total lipid content. For example if the overexpression of a gene participating in the biosynthesis of triacylglycerols in the seed results in an increase in total lipid content in the seed or leaf this seed will also show an increased glycerol content after total lipid extraction and hydrolysis of the lipids.

The same is valid for glycerophosphodiesters, preferably GPG, which are formed by deacylation of phspholipids. Therefore the analysis of the glycerophosphodiesters content in cells, tissues or plant parts like seeds and leaves after total lipid extraction directly correlates with the analysis of the total phospholipid content.

An increased glycerol, glycerolphosphate, preferably G3P, and/or glycerophosphodiesters, preferably GPG content normally means an increased of the total glycerol, glycerolphosphate, preferably G3P, and/or glycerophosphodiesters, preferably GPG content. However, a increased glycerol, glycerolphosphate, preferably G3P, and/or glycerophosphodiesters, preferably GPG content also means, in particular, a modified content of the above-described derivates of glycerol, glycerolphosphate, preferably G3P, and/or glycerophosphodiesters, preferably GPG containing compounds.

Accordingly, the present invention relates to a process for the production of glycerol phosphate (lipid fraction), which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of At1g23110-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of eukaryotic translation initiation factor 5 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol phosphate (lipid fraction), which comprises
(a) increasing or generating one or more activities selected from the group consisting of eukaryotic translation initiation factor 5 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of triacylglycerol, which comprises
(a) increasing or generating one or more activities selected from the group consisting of At1g67340-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of glycerol (lipid fraction), which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol phosphate (lipid fraction), which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of CBL-interacting protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of phosphatidylinositol 3- and 4-kinase family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of triacylglycerol, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of glycerol (lipid fraction), which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol (lipid fraction), which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of triacylglycerol, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of glycerol (lipid fraction), which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of triacylglycerol, which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of glycerol phosphate (lipid fraction), which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of calcium-dependent protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of triacylglycerol, which comprises
(a) increasing or generating one or more activities selected from the group consisting of heat shock transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of glycerol (lipid fraction), which comprises
(a) increasing or generating one or more activities selected from the group consisting of monothiol glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol phosphate (lipid fraction), which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol phosphate (lipid fraction), which comprises
(a) increasing or generating one or more activities selected from the group consisting of protein phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol phosphate (lipid fraction), which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of triacylglycerol, which comprises
(a) increasing or generating one or more activities selected from the group consisting of Glycine cleavage system T aminomethyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of glycerol phosphate (lipid fraction), which comprises
(a) increasing or generating one or more activities selected from the group consisting of sec-independent protein translocase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol (lipid fraction), which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of transcriptional regulator protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of beta-hydroxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of triacylglycerol, which comprises
(a) increasing or generating one or more activities selected from the group consisting of beta-hydroxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol phosphate (lipid fraction), which comprises
(a) increasing or generating one or more activities selected from the group consisting of ThiF family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol (lipid fraction), which comprises
(a) increasing or generating one or more activities selected from the group consisting of recombinase A in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of NADH-quinone oxidoreductase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of triacylglycerol, which comprises
(a) increasing or generating one or more activities selected from the group consisting of hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of glycerol phosphate (lipid fraction), which comprises
(a) increasing or generating one or more activities selected from the group consisting of HesB/YadR/YfhF family protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of triacylglycerol, which comprises
(a) increasing or generating one or more activities selected from the group consisting of threonine synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of glycerol phosphate (lipid fraction), which comprises
(a) increasing or generating one or more activities selected from the group consisting of b0050-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of serine protease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of triacylglycerol, which comprises
(a) increasing or generating one or more activities selected from the group consisting of serine protease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of ATP-binding component of a transport system in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating one or more activities selected from the group consisting of b0518-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of phosphoglyceromutase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (polar fraction), which comprises
(a) increasing or generating one or more activities selected from the group consisting of putrescine transport system permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

Accordingly, the present invention relates to a process for the production of glycerol (lipid fraction), which comprises
(a) increasing or generating one or more activities selected from the group consisting of methylglyoxal synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol (polar fraction), which comprises
(a) increasing or generating one or more activities selected from the group consisting of methylglyoxal synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of b0970-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of b1003-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of 3-oxoacyl-[acyl-carrier-protein] synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating one or more activities selected from the group consisting of sodium/proton antiporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of triacylglycerol, which comprises
(a) increasing or generating one or more activities selected from the group consisting of b1234-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of glycerol (lipid fraction), which comprises
(a) increasing or generating one or more activities selected from the group consisting of cardiolipin synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol phosphate (lipid fraction), which comprises
(a) increasing or generating one or more activities selected from the group consisting of b1330-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of ATP-dependent RNA helicase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of phenylacetic acid degradation protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of lipoprotein precursor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of b1522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of lysozyme in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of b1556-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of triacylglycerol, which comprises
(a) increasing or generating one or more activities selected from the group consisting of b1583-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of Fe—S subunit of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating one or more activities selected from the group consisting of acid shock protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of b1670-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of b1672-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of triacylglycerol, which comprises
(a) increasing or generating one or more activities selected from the group consisting of b1703-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of phosphotransferase system component in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of triacylglycerol, which comprises
(a) increasing or generating one or more activities selected from the group consisting of B1795-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of glycerol-3-phosphate (polar fraction), which comprises
(a) increasing or generating one or more activities selected from the group consisting of L-serine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol-3-phosphate (polar fraction) or a composition comprising glycerol-3-phosphate (polar fraction) in said non-human organism or in the culture medium surrounding said non-human organism.

Accordingly, the present invention relates to a process for the production of glycerol phosphate (lipid fraction), which comprises
(a) increasing or generating one or more activities selected from the group consisting of b1837-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of triacylglycerol, which comprises
(a) increasing or generating one or more activities selected from the group consisting of trehalose-phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of glycerol (lipid fraction), which comprises
(a) increasing or generating one or more activities selected from the group consisting of imidazole glycerol phosphate synthase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol phosphate (lipid fraction), which comprises
(a) increasing or generating one or more activities selected from the group consisting of b2032-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol (lipid fraction), which comprises
(a) increasing or generating one or more activities selected from the group consisting of b2099-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of b2139-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating one or more activities selected from the group consisting of ABC transporter permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol phosphate (lipid fraction), which comprises
(a) increasing or generating one or more activities selected from the group consisting of ion-transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of b2399-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating one or more activities selected from the group consisting of b2399-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol (lipid fraction), which comprises
(a) increasing or generating one or more activities selected from the group consisting of ethanolamine utilization protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol phosphate (lipid fraction), which comprises
(a) increasing or generating one or more activities selected from the group consisting of ethanolamine utilization protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol (lipid fraction), which comprises
(a) increasing or generating one or more activities selected from the group consisting of ethanolamine utilization protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of b2474-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating one or more activities selected from the group consisting of b2474-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol (lipid fraction), which comprises
(a) increasing or generating one or more activities selected from the group consisting of putative transport system permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol phosphate (lipid fraction), which comprises
(a) increasing or generating one or more activities selected from the group consisting of putative transport system permease protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of b2613-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (polar fraction), which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

Accordingly, the present invention relates to a process for the production of glycerol (lipid fraction), which comprises
(a) increasing or generating one or more activities selected from the group consisting of b2673-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol phosphate (lipid fraction), which comprises
(a) increasing or generating one or more activities selected from the group consisting of b2673-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of murein transglycosylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of triacylglycerol, which comprises
(a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of glycerol (lipid fraction), which comprises
(a) increasing or generating one or more activities selected from the group consisting of 2-C-methyl-D-erythritol 4-phosphate cytidylyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of triacylglycerol, which comprises
(a) increasing or generating one or more activities selected from the group consisting of amino-acid acetyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of b3083-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of threonine/serine transporter in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of b3160-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of argininosuccinate synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol-3-phosphate (polar fraction), which comprises
(a) increasing or generating one or more activities selected from the group consisting of acetyl CoA carboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol-3-phosphate (polar fraction) or a composition comprising glycerol-3-phosphate (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating one or more activities selected from the group consisting of methyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of FKBP-type peptidyl-prolyl cis-trans isomerase (rotamase) in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of high-affinity branched-chain amino acid transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of b3777-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of b3791-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of enterobacterial common antigen polymerase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of triacylglycerol, which comprises
(a) increasing or generating one or more activities selected from the group consisting of b3989-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of b4029-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of triacylglycerol, which comprises
(a) increasing or generating one or more activities selected from the group consisting of b4050-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of lysyl-tRNA synthetase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol-3-phosphate (polar fraction), which comprises
(a) increasing or generating one or more activities selected from the group consisting of purine-nucleoside phosphorylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol-3-phosphate (polar fraction) or a composition comprising glycerol-3-phosphate (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol phosphate (lipid fraction), which comprises
(a) increasing or generating one or more activities selected from the group consisting of glutaredoxin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of GM02LC17485-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol phosphate (lipid fraction), which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol (polar fraction), which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

Accordingly, the present invention relates to a process for the production of glycerol phosphate (lipid fraction), which comprises
(a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of nitrate/nitrite transport protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol phosphate (lipid fraction), which comprises
(a) increasing or generating one or more activities selected from the group consisting of carbamoyl-phosphate synthase subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol (lipid fraction), which comprises
(a) increasing or generating one or more activities selected from the group consisting of CDP-diacylglycerol-glycerol-3-phosphate 3-phosphatidyltransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of adenylate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating one or more activities selected from the group consisting of acyl transferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol phosphate (lipid fraction), which comprises
(a) increasing or generating one or more activities selected from the group consisting of coproporphyrinogen III oxidase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol (lipid fraction), which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol phosphate (lipid fraction), which comprises
(a) increasing or generating one or more activities selected from the group consisting of oxidoreductase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol (polar fraction), which comprises
(a) increasing or generating one or more activities selected from the group consisting of Mg-protoporphyrin IX monomethyl ester oxidative cyclase 66 kD subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of dihydrolipoamide dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol phosphate (lipid fraction), which comprises
(a) increasing or generating one or more activities selected from the group consisting of aromatic acid decarboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol phosphate (lipid fraction), which comprises
(a) increasing or generating one or more activities selected from the group consisting of phosphate import ATP-binding protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol phosphate (lipid fraction), which comprises
(a) increasing or generating one or more activities selected from the group consisting of arginine decarboxylase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol phosphate (lipid fraction), which comprises
(a) increasing or generating one or more activities selected from the group consisting of carbohydrate kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of precorrin-8X methylmutase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of NADH dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating one or more activities selected from the group consisting of glutamate 5-kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol (lipid fraction), which comprises
(a) increasing or generating one or more activities selected from the group consisting of threonine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol phosphate (lipid fraction), which comprises
(a) increasing or generating one or more activities selected from the group consisting of threonine dehydratase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of photosystem I reaction centre subunit in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of triacylglycerol, which comprises
(a) increasing or generating one or more activities selected from the group consisting of cell division protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of glycerol phosphate (lipid fraction), which comprises
(a) increasing or generating one or more activities selected from the group consisting of metal-dependent hydrolase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol (lipid fraction), which comprises
(a) increasing or generating one or more activities selected from the group consisting of TTC1386-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol-3-phosphate (polar fraction), which comprises
(a) increasing or generating one or more activities selected from the group consisting of YAL013W-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol-3-phosphate (polar fraction) or a composition comprising glycerol-3-phosphate (polar fraction) in said non-human organism or in the culture medium surrounding said non-human organism.

Accordingly, the present invention relates to a process for the production of glycerol (lipid fraction), which comprises
(a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol phosphate (lipid fraction), which comprises
(a) increasing or generating one or more activities selected from the group consisting of acid phosphatase precursor in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol (lipid fraction), which comprises
(a) increasing or generating one or more activities selected from the group consisting of cell division control protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol (polar fraction), which comprises
(a) increasing or generating one or more activities selected from the group consisting of cell division control protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of ycr102c-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of ydr273w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol phosphate (lipid fraction), which comprises
(a) increasing or generating one or more activities selected from the group consisting of molecular chaperone portein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of triacylglycerol, which comprises
(a) increasing or generating one or more activities selected from the group consisting of YFL019C-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of alcohol dehydrogenase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating one or more activities selected from the group consisting of biotin synthase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of triacylglycerol, which comprises
(a) increasing or generating one or more activities selected from the group consisting of mitochondrial processing protease in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of YHR202W-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (polar fraction), which comprises
(a) increasing or generating one or more activities selected from the group consisting of (DL)-glycerol-3-phosphatase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of YIL083C-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol phosphate (lipid fraction), which comprises
(a) increasing or generating one or more activities selected from the group consisting of YJL062W-A-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of DnaJ-like chaperone in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of polygalacturonase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (polar fraction), which comprises
(a) increasing or generating one or more activities selected from the group consisting of vacuolar sorting protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of YLR099C-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating one or more activities selected from the group consisting of cyclin in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol (lipid fraction), which comprises
(a) increasing or generating one or more activities selected from the group consisting of ylr326w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol phosphate (lipid fraction), which comprises
(a) increasing or generating one or more activities selected from the group consisting of ylr326w-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol (lipid fraction), which comprises
(a) increasing or generating one or more activities selected from the group consisting of heat shock protein Hsp40 in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol (lipid fraction), which comprises
(a) increasing or generating one or more activities selected from the group consisting of phosphotransferase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol (lipid fraction), which comprises
(a) increasing or generating one or more activities selected from the group consisting of Elongation factor Tu in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of YPL162C. protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating one or more activities selected from the group consisting of mitogen-activated protein kinase in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of Zm_{—}4842_BE510522-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating one or more activities selected from the group consisting of 49747384_SOYBEAN-protein in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

An embodiment of the present invention relates to a process for the production of bis-glyceryl phosphate (polar fraction), which comprises
(a1) increasing or generating the activity of a polypeptide comprising a polypeptide as depicted in a respective line in column 5 or 8 of Table II, application no. 19, or a homolog or a fragment thereof, a consensus sequence or at least one polypeptide motif as depicted in the respective line in column 8 of Table IV, application no. 19, whereby the respective line disclose in column 7 the fine chemical bis-glyceryl phosphate (polar fraction); or
(a2) increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical bis-glyceryl phosphate (polar fraction); or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of bis-glyceryl phosphate (polar fraction) or a composition comprising bis-glyceryl phosphate (polar fraction) in said non-human organism or in the culture medium surrounding said non-human organism.

An embodiment of the present invention relates to a process for the production of glycerol (lipid fraction), which comprises
(a1) increasing or generating the activity of a polypeptide comprising a polypeptide as depicted in a respective line in column 5 or 8 of Table II, application no. 19, or a homolog or a fragment thereof, a consensus sequence or at least one polypeptide motif as depicted in the respective line in column 8 of Table IV, application no. 19, whereby the respective line disclose in column 7 the fine chemical glycerol (lipid fraction); or
(a2) increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical glycerol (lipid fraction); or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of glycerol (lipid fraction) or a composition comprising glycerol (lipid fraction) in said non-human organism or in the culture medium surrounding said non-human organism.

An embodiment of the present invention relates to a process for the production of glycerol (polar fraction), which comprises
(a1) increasing or generating the activity of a polypeptide comprising a polypeptide as depicted in a respective line in column 5 or 8 of Table II, application no. 19, or a homolog or a fragment thereof, a consensus sequence or at least one polypeptide motif as depicted in the respective line in column 8 of Table IV, application no. 19, whereby the respective line disclose in column 7 the fine chemical glycerol (polar fraction); or
(a2) increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical glycerol (polar fraction); or
(a3) increasing or generating the activity of a functional equivalent of (a1) or (a2); in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of glycerol (polar fraction) or a composition comprising glycerol (polar fraction) in said non-human organism or in the culture medium surrounding said non-human organism.

An embodiment of the present invention relates to a process for the production of glycerol phosphate (lipid fraction), which comprises
(a1) increasing or generating the activity of a polypeptide comprising a polypeptide as depicted in a respective line in column 5 or 8 of Table II, application no. 19, or a homolog or a fragment thereof, a consensus sequence or at least one polypeptide motif as depicted in the respective line in column 8 of Table IV, application no. 19, whereby the respective line disclose in column 7 the fine chemical glycerol phosphate (lipid fraction); or
(a2) increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical glycerol phosphate (lipid fraction); or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of glycerol phosphate (lipid fraction) or a composition comprising glycerol phosphate (lipid fraction) in said non-human organism or in the culture medium surrounding said non-human organism.

An embodiment of the present invention relates to a process for the production of glycerol-3-phosphate (polar fraction), which comprises
(a1) increasing or generating the activity of a polypeptide comprising a polypeptide as depicted in a respective line in column 5 or 8 of Table II, application no. 19, or a homolog or a fragment thereof, a consensus sequence or at least one polypeptide motif as depicted in the respective line in column 8 of Table IV, application no. 19, whereby the respective line disclose in column 7 the fine chemical glycerol-3-phosphate (polar fraction); or
(a2) increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical glycerol-3-phosphate (polar fraction); or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of glycerol-3-phosphate (polar fraction) or a composition comprising glycerol-3-phosphate (polar fraction) in said non-human organism or in the culture medium surrounding said non-human organism

An embodiment of the present invention relates to a process for the production of triacylglycerol, which comprises
(a1) increasing or generating the activity of a polypeptide comprising a polypeptide as depicted in a respective line in column 5 or 8 of Table II, application no. 19, or a homolog or a fragment thereof, a consensus sequence or at least one polypeptide motif as depicted in the respective line in column 8 of Table IV, application no. 19, whereby the respective line disclose in column 7 the fine chemical triacylglycerol; or
(a2) increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical triacylglycerol; or
(a3) increasing or generating the activity of a functional equivalent of (a1) or (a2);
in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of triacylglycerol or a composition comprising triacylglycerol in said non-human organism or in the culture medium surrounding said non-human organism.

Accordingly, the increase or generation of one or more said activities is for example conferred by one or more expression products of said nucleic acid molecule, e.g. proteins. Accordingly, in the present invention described above, the increase or generation of one or more said activities is for example conferred by one or more protein(s) each comprising a polypeptide selected from the group as depicted in Table II, application no. 19, column 5 and 8, or a homolog or a fragment thereof.

The process of the invention comprises in one embodiment the following steps:
(i) increasing or generating of the expression of; and/or
(ii) increasing or generating the expression of an expression product of; and/or
(iii) increasing or generating one or more activities of an expression product encoded by;
   at least one nucleic acid molecule (in the following “Fine Chemical Related Protein (FCRP)”-encoding gene or “FCRP”-gene) comprising a nucleic acid molecule selected from the group consisting of:
(a) a nucleic acid molecule encoding the polypeptide shown in column 5 or 8 of Table II, preferably Table II B, application no. 19, or a homolog or a fragment thereof;
(b) a nucleic acid molecule shown in column 5 or 8 of Table I, preferably Table I B, application no. 19, or a homolog or a fragment thereof (preferably the coding region thereof);
(c) a nucleic acid molecule, which, as a result of the degeneracy of the genetic code, can be derived from a polypeptide sequence depicted in column 5 or 8 of Table II, preferably Table II B, application no. 19;
(d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 19, or the coding region thereof;
(e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, application no. 19;
(i) a nucleic acid molecule encoding a polypeptide having the activity represented by a respective protein as depicted in column 5 of Table II, application no. 19;
(j) a nucleic acid molecule which comprises a polynucleotide, which is obtained by amplifying a cDNA library or a genomic library using the primers as depicted in column 8 of Table III, application no. 19; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto.
   (All the references to Tables I to IV are references to the corresponding lines of Tables I to IV.)

In a preferred embodiment thereof said nucleic acid molecule encodes a polypeptide which has the activity of the polypeptide represented by a protein comprising a polypeptide as depicted in the corresponding hit in column 5 of Table II, application no. 19.

In another preferred embodiment thereof said nucleic acid molecule confers a generation or an increase of the respective fine chemical in a non-human organism or a part thereof as compared to a corresponding wild-type non-human organism or part thereof.

In another preferred embodiment thereof said nucleic acid molecule encodes a polypeptide which has the activity of the polypeptide represented by a protein as depicted in the corresponding hit in column 5 of Table II, application no. 19, and confers a generation or an increase of the respective fine chemical in a non-human organism or a part thereof as compared to a corresponding wild-type non-human organism or part thereof.

Accordingly, the genes of the present invention or used in accordance with the present invention, which respectively encode a protein having an activity of (DL)-glycerol-3-phosphatase, 2-C-methyl-D-erythritol 4-phosphate cytidylyltransferase, 3-oxoacyl-[acyl-carrier-protein] synthase, 49747384_SOYBEAN-protein, ABC transporter permease protein, acetyl CoA carboxylase, acid phosphatase precursor, acid shock protein, acyl transferase, adenylate kinase, alcohol dehydrogenase, amino-acid acetyltransferase, arginine decarboxylase, argininosuccinate synthetase, aromatic acid decarboxylase, At1g23110-protein, At1g67340-protein, ATP-binding component of a transport system, ATP-dependent RNA helicase, b0050-protein, b0518-protein, b0970-protein, b1003-protein, b1234-protein, b1330-protein, b1522-protein, b1556-protein, b1583-protein, b1670-protein, b1672-protein, b1703-protein, B1795-protein, b1837-protein, b2032-protein, b2099-protein, b2139-protein, b2399-protein, b2474-protein, b2613-protein, b2673-protein, b3083-protein, b3160-protein, b3777-protein, b3791-protein, b3989-protein, b4029-protein, b4050-protein, beta-hydroxylase, biotin synthase, calcium-dependent protein kinase, carbamoyl-phosphate synthase subunit, carbohydrate kinase, cardiolipin synthase, CBL-interacting protein kinase, CDP-diacylglycerol-glycerol-3-phosphate 3-phosphatidyltransferase, cell division control protein, cell division protein, coproporphyrinogen III oxidase, cyclin, cyclin D , dihydrolipoamide dehydrogenase, DnaJ-like chaperone, Elongation factor Tu, enterobacterial common antigen polymerase, ethanolamine utilization protein, eukaryotic translation initiation factor 5, Fe—S subunit of oxidoreductase, FKBP-type peptidyl-prolyl cis-trans isomerase (rotamase), glutamate 5-kinase, glutaredoxin, Glycine cleavage system T aminomethyltransferase, GM02LC17485-protein, heat shock protein Hsp40, heat shock transcription factor, HesB/YadR/YfhF family protein, high-affinity branched-chain amino acid transport protein, hydrolase, imidazole glycerol phosphate synthase subunit, ion-transport protein, lipoprotein precursor, L-serine dehydratase, lysozyme, lysyl-tRNA synthetase, metal-dependent hydrolase, methylglyoxal synthase, methyltransferase, Mg-protoporphyrin IX monomethyl ester oxidative cyclase 66 kD subunit, mitochondrial processing protease, mitogen-activated protein kinase, molecular chaperone portein, monothiol glutaredoxin, murein transglycosylase, NADH dehydrogenase, NADH-quinone oxidoreductase subunit, nitrate/nitrite transport protein, oxidoreductase, phenylacetic acid degradation protein, phosphate import ATP-binding protein, phosphatidylinositol 3- and 4-kinase family protein, phosphoglyceromutase, phosphotransferase, phosphotransferase system component, photosystem I reaction centre subunit, polygalacturonase, precorrin-8X methylmutase, protein phosphatase, purine-nucleoside phosphorylase, putative transport system permease protein, putrescine transport system permease protein, pyruvate kinase, recombinase A, sec-independent protein translocase, serine protease, sodium/proton antiporter, ThiF family protein, threonine dehydratase, threonine synthase, threonine/serine transporter, transcription factor, transcriptional regulator, transcriptional regulator protein, trehalose-phosphatase, TTC1386-protein, vacuolar sorting protein, YAL013W-protein, ycr102c-protein, ydr273w-protein, YFL019C-protein, YHR202W-protein, YIL083C-protein, YJL062W-A-protein, YLR099C-protein, ylr326w-protein, YPL162C.protein, or Zm_{—}4842_BE510522-protein, which respectively encode a protein comprising a polypeptide encoded by a nucleic acid sequence as shown in Table I, application no. 19, column 5 or 8, (preferably the coding region thereof), or a homolog or a fragment thereof, which respectively encode a protein comprising a polypeptide as depicted in Table II, application no. 19, column 5 or 8, or a homolg or a fragment thereof, and/or which respectively can be amplified with the primer set shown in Table III, application no. 19, column 8, are also referred to as “FCRP genes”.

Proteins or polypeptides encoded by “FCRP-genes” are referred to as “Fine Chemical Related Proteins” or “FCRP”. For the purposes of the description of the present invention, the respective protein having an activity of (DL)-glycerol-3-phosphatase, 2-C-methyl-D-erythritol 4-phosphate cytidylyltransferase, 3-oxoacyl-[acyl-carrier-protein] synthase, 49747384_SOYBEAN-protein, ABC transporter permease protein, acetyl CoA carboxylase, acid phosphatase precursor, acid shock protein, acyl transferase, adenylate kinase, alcohol dehydrogenase, amino-acid acetyltransferase, arginine decarboxylase, argininosuccinate synthetase, aromatic acid decarboxylase, At1g23110-protein, At1g67340-protein, ATP-binding component of a transport system, ATP-dependent RNA helicase, b0050-protein, b0518-protein, b0970-protein, b1003-protein, b1234-protein, b1330-protein, b1522-protein, b1556-protein, b1583-protein, b1670-protein, b1672-protein, b1703-protein, B1795-protein, b1837-protein, b2032-protein, b2099-protein, b2139-protein, b2399-protein, b2474-protein, b2613-protein, b2673-protein, b3083-protein, b3160-protein, b3777-protein, b3791-protein, b3989-protein, b4029-protein, b4050-protein, beta-hydroxylase, biotin synthase, calcium-dependent protein kinase, carbamoyl-phosphate synthase subunit, carbohydrate kinase, cardiolipin synthase, CBL-interacting protein kinase, CDP-diacylglycerol-glycerol-3-phosphate 3-phosphatidyltransferase, cell division control protein, cell division protein, coproporphyrinogen III oxidase, cyclin, cyclin D , dihydrolipoamide dehydrogenase, DnaJ-like chaperone, Elongation factor Tu, enterobacterial common antigen polymerase, ethanolamine utilization protein, eukaryotic translation initiation factor 5, Fe—S subunit of oxidoreductase, FKBP-type peptidyl-prolyl cis-trans isomerase (rotamase), glutamate 5-kinase, glutaredoxin, Glycine cleavage system T aminomethyltransferase, GM02LC17485-protein, heat shock protein Hsp40, heat shock transcription factor, HesB/YadR/YfhF family protein, high-affinity branched-chain amino acid transport protein, hydrolase, imidazole glycerol phosphate synthase subunit, ion-transport protein, lipoprotein precursor, L-serine dehydratase, lysozyme, lysyl-tRNA synthetase, metal-dependent hydrolase, methylglyoxal synthase, methyltransferase, Mg-protoporphyrin IX monomethyl ester oxidative cyclase 66 kD subunit, mitochondrial processing protease, mitogen-activated protein kinase, molecular chaperone portein, monothiol glutaredoxin, murein transglycosylase, NADH dehydrogenase, NADH-quinone oxidoreductase subunit, nitrate/nitrite transport protein, oxidoreductase, phenylacetic acid degradation protein, phosphate import ATP-binding protein, phosphatidylinositol 3- and 4-kinase family protein, phosphoglyceromutase, phosphotransferase, phosphotransferase system component, photosystem I reaction centre subunit, polygalacturonase, precorrin-8X methylmutase, protein phosphatase, purine-nucleoside phosphorylase, putative transport system permease protein, putrescine transport system permease protein, pyruvate kinase, recombinase A, sec-independent protein translocase, serine protease, sodium/proton antiporter, ThiF family protein, threonine dehydratase, threonine synthase, threonine/serine transporter, transcription factor, transcriptional regulator, transcriptional regulator protein, trehalose-phosphatase, TTC1386-protein, vacuolar sorting protein, YAL013W-protein, ycr102c-protein, ydr273w-protein, YFL0190-protein, YHR202W-protein, YIL083C-protein, YJL062W-A-protein, YLR099C-protein, ylr326w-protein, YPL162C.protein, or Zm_{—}4842_BE510522-protein, the respective protein comprising a polypeptide encoded by one or more respective nucleic acid sequences as shown in Table I, application no. 19, column 5 or 8, (preferably the coding region thereof), or a homolog or fragment thereof, the respective protein comprising a respective polypeptide as depicted in Table II, application no. 19, column 5 or 8, or a homolog or fragment thereof, the respective protein comprising a sequence corresponding to the consensus sequence as shown in Table IV, application no. 19, column 8, and/or the respective protein comprising at least one polypeptide motif as shown in Table IV, application no. 19, column 8 is also referred to as Fine Chemical Related Protein” or “FCRP”.

Thus, in one embodiment, the present invention provides a process of the production of bis-glyceryl phosphate (polar fraction), glycerol (lipid fraction), glycerol (polar fraction), glycerol phosphate (lipid fraction), glycerol-3-phosphate (polar fraction), and/or triacylglycerol, by increasing or generating one or more activities, especially selected from the group consisting of (DL)-glycerol-3-phosphatase, 2-C-methyl-D-erythritol 4-phosphate cytidylyltransferase, 3-oxoacyl-[acyl-carrier-protein] synthase, 49747384_SOYBEAN-protein, ABC transporter permease protein, acetyl CoA carboxylase, acid phosphatase precursor, acid shock protein, acyl transferase, adenylate kinase, alcohol dehydrogenase, amino-acid acetyltransferase, arginine decarboxylase, argininosuccinate synthetase, aromatic acid decarboxylase, At1g23110-protein, At1g67340-protein, ATP-binding component of a transport system, ATP-dependent RNA helicase, b0050-protein, b0518-protein, b0970-protein, b1003-protein, b1234-protein, b1330-protein, b1522-protein, b1556-protein, b1583-protein, b1670-protein, b1672-protein, b1703-protein, B1795-protein, b1837-protein, b2032-protein, b2099-protein, b2139-protein, b2399-protein, b2474-protein, b2613-protein, b2673-protein, b3083-protein, b3160-protein, b3777-protein, b3791-protein, b3989-protein, b4029-protein, b4050-protein, beta-hydroxylase, biotin synthase, calcium-dependent protein kinase, carbamoyl-phosphate synthase subunit, carbohydrate kinase, cardiolipin synthase, CBL-interacting protein kinase, CDP-diacylglycerol-glycerol-3-phosphate 3-phosphatidyltransferase, cell division control protein, cell division protein, coproporphyrinogen III oxidase, cyclin, cyclin D , dihydrolipoamide dehydrogenase, DnaJ-like chaperone, Elongation factor Tu, enterobacterial common antigen polymerase, ethanolamine utilization protein, eukaryotic translation initiation factor 5, Fe—S subunit of oxidoreductase, FKBP-type peptidyl-prolyl cis-trans isomerase (rotamase), glutamate 5-kinase, glutaredoxin, Glycine cleavage system T aminomethyltransferase, GM02LC17485-protein, heat shock protein Hsp40, heat shock transcription factor, HesB/YadR/YfhF family protein, high-affinity branched-chain amino acid transport protein, hydrolase, imidazole glycerol phosphate synthase subunit, ion-transport protein, lipoprotein precursor, L-serine dehydratase, lysozyme, lysyl-tRNA synthetase, metal-dependent hydrolase, methylglyoxal synthase, methyltransferase, Mg-protoporphyrin IX monomethyl ester oxidative cyclase 66 kD subunit, mitochondrial processing protease, mitogen-activated protein kinase, molecular chaperone portein, monothiol glutaredoxin, murein transglycosylase, NADH dehydrogenase, NADH-quinone oxidoreductase subunit, nitrate/nitrite transport protein, oxidoreductase, phenylacetic acid degradation protein, phosphate import ATP-binding protein, phosphatidylinositol 3- and 4-kinase family protein, phosphoglyceromutase, phosphotransferase, phosphotransferase system component, photosystem I reaction centre subunit, polygalacturonase, precorrin-8X methylmutase, protein phosphatase, purine-nucleoside phosphorylase, putative transport system permease protein, putrescine transport system permease protein, pyruvate kinase, recombinase A, sec-independent protein translocase, serine protease, sodium/proton antiporter, ThiF family protein, threonine dehydratase, threonine synthase, threonine/serine transporter, transcription factor, transcriptional regulator, transcriptional regulator protein, trehalose-phosphatase, TTC1386-protein, vacuolar sorting protein, YAL013W-protein, ycr102c-protein, ydr273w-protein, YFL019C-protein, YHR202W-protein, YIL083C-protein, YJL062W-A-protein, YLR099C-protein, ylr326w-protein, YPL162C.protein, and Zm_{—}4842_BE510522-protein, which is conferred by one or more FCRPs or the gene product of one or more FCRP-genes, for example by the gene product of a nucleic acid sequences comprising a polynucleotide selected from the group as shown in Table I, application no. 19, column 5 or 8, (preferably by the coding region thereof), or a homolog or a fragment thereof, e.g. or by one or more proteins each comprising a polypeptide encoded by one or more nucleic acid sequences selected from the group as shown in Table I, application no. 19, column 5 or 8, (preferably by the coding region thereof), or a homolog or a fragment thereof, or by one or more protein(s) each comprising a polypeptide selected from the group as depicted in Table II, application no. 19, column 5 and 8, or a homolog thereof, or a protein comprising a sequence corresponding to the consensus sequence or comprising at least one polypeptide motif as shown in Table IV, application no. 19, column 8.

for the disclosure of this paragraph see [0025.1.7.7] above

In an embodiment, the process comprises increasing or generating the activity of one or more polypeptides having said activity, e.g.

by generating or increasing the amount and/or specific activity in the cell or a compartment of a cell of one of more FCRP, especially selected from the group consisting of (DL)-glycerol-3-phosphatase, 2-C-methyl-D-erythritol 4-phosphate cytidylyltransferase, 3-oxoacyl-[acyl-carrier-protein] synthase, 49747384_SOYBEAN-protein, ABC transporter permease protein, acetyl CoA carboxylase, acid phosphatase precursor, acid shock protein, acyl transferase, adenylate kinase, alcohol dehydrogenase, amino-acid acetyltransferase, arginine decarboxylase, argininosuccinate synthetase, aromatic acid decarboxylase, At1g23110-protein, At1g67340-protein, ATP-binding component of a transport system, ATP-dependent RNA helicase, b0050-protein, b0518-protein, b0970-protein, b1003-protein, b1234-protein, b1330-protein, b1522-protein, b1556-protein, b1583-protein, b1670-protein, b1672-protein, b1703-protein, B1795-protein, b1837-protein, b2032-protein, b2099-protein, b2139-protein, b2399-protein, b2474-protein, b2613-protein, b2673-protein, b3083-protein, b3160-protein, b3777-protein, b3791-protein, b3989-protein, b4029-protein, b4050-protein, beta-hydroxylase, biotin synthase, calcium-dependent protein kinase, carbamoyl-phosphate synthase subunit, carbohydrate kinase, cardiolipin synthase, CBL-interacting protein kinase, CDP-diacylglycerol-glycerol-3-phosphate 3-phosphatidyltransferase, cell division control protein, cell division protein, coproporphyrinogen III oxidase, cyclin, cyclin D , dihydrolipoamide dehydrogenase, DnaJ-like chaperone, Elongation factor Tu, enterobacterial common antigen polymerase, ethanolamine utilization protein, eukaryotic translation initiation factor 5, Fe—S subunit of oxidoreductase, FKBP-type peptidyl-prolyl cis-trans isomerase (rotamase), glutamate 5-kinase, glutaredoxin, Glycine cleavage system T aminomethyltransferase, GM02LC17485-protein, heat shock protein Hsp40, heat shock transcription factor, HesB/YadR/YfhF family protein, high-affinity branched-chain amino acid transport protein, hydrolase, imidazole glycerol phosphate synthase subunit, ion-transport protein, lipoprotein precursor, L-serine dehydratase, lysozyme, lysyl-tRNA synthetase, metal-dependent hydrolase, methylglyoxal synthase, methyltransferase, Mg-protoporphyrin IX monomethyl ester oxidative cyclase 66 kD subunit, mitochondrial processing protease, mitogen-activated protein kinase, molecular chaperone portein, monothiol glutaredoxin, murein transglycosylase, NADH dehydrogenase, NADH-quinone oxidoreductase subunit, nitrate/nitrite transport protein, oxidoreductase, phenylacetic acid degradation protein, phosphate import ATP-binding protein, phosphatidylinositol 3- and 4-kinase family protein, phosphoglyceromutase, phosphotransferase, phosphotransferase system component, photosystem I reaction centre subunit, polygalacturonase, precorrin-8X methylmutase, protein phosphatase, purine-nucleoside phosphorylase, putative transport system permease protein, putrescine transport system permease protein, pyruvate kinase, recombinase A, sec-independent protein translocase, serine protease, sodium/proton antiporter, ThiF family protein, threonine dehydratase, threonine synthase, threonine/serine transporter, transcription factor, transcriptional regulator, transcriptional regulafor protein, trehalose-phosphatase, TTC1386-protein, vacuolar sorting protein, YAL013W-protein, ycr102c-protein, ydr273w-protein, YFL019C-protein, YHR202W-protein, YIL083C-protein, YJL062W-A-protein, YLR099C-protein, ylr326w-protein, YPL162C.protein, and Zm_{—}4842_BE510522-protein, for example of the respective polypeptide as depicted in Table II, application no. 19, column 5 and 8, or a homolog or a fragment thereof, or the respective polypeptide comprising a sequence corresponding to the consensus sequences as shown in Table IV, application no. 19, column 8, or the respective polypeptide comprising at least one polypeptide motif as depicted in Table IV, application no. 19, column 8.

A further embodiment of the present invention relates to a process for the production of glycerol phosphate (lipid fraction), which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of bis-glyceryl phosphate (polar fraction), which comprises
(a) increasing or generating the activity of a At1g23110-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a eukaryotic translation initiation factor 5 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol phosphate (lipid fraction), which comprises
(a) increasing or generating the activity of a eukaryotic translation initiation factor 5 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of triacylglycerol, which comprises
(a) increasing or generating the activity of a At1g67340-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of glycerol (lipid fraction), which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of bis-glyceryl phosphate (polar fraction), which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol phosphate (lipid fraction), which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of bis-glyceryl phosphate (polar fraction), which comprises
(a) increasing or generating the activity of a CBL-interacting protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a phosphatidylinositol 3- and 4-kinase family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of triacylglycerol, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of glycerol (lipid fraction), which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol (lipid fraction), which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of triacylglycerol, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of glycerol (lipid fraction), which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of triacylglycerol, which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of glycerol phosphate (lipid fraction), which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of bis-glyceryl phosphate (polar fraction), which comprises
(a) increasing or generating the activity of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a calcium-dependent protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of triacylglycerol, which comprises
(a) increasing or generating the activity of a heat shock transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of glycerol (lipid fraction), which comprises
(a) increasing or generating the activity of a monothiol glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol phosphate (lipid fraction), which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol phosphate (lipid fraction), which comprises
(a) increasing or generating the activity of a protein phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol phosphate (lipid fraction), which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of triacylglycerol, which comprises
(a) increasing or generating the activity of a Glycine cleavage system T aminomethyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of glycerol phosphate (lipid fraction), which comprises
(a) increasing or generating the activity of a sec-independent protein translocase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol (lipid fraction), which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of bis-glyceryl phosphate (polar fraction), which comprises
(a) increasing or generating the activity of a transcriptional regulator protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a beta-hydroxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of triacylglycerol, which comprises
(a) increasing or generating the activity of a beta-hydroxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of bis-glyceryl phosphate (polar fraction), which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol phosphate (lipid fraction), which comprises
(a) increasing or generating the activity of a ThiF family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol (lipid fraction), which comprises
(a) increasing or generating the activity of a recombinase A non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of bis-glyceryl phosphate (polar fraction), which comprises
(a) increasing or generating the activity of a NADH-quinone oxidoreductase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of triacylglycerol, which comprises
(a) increasing or generating the activity of a hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of glycerol phosphate (lipid fraction), which comprises
(a) increasing or generating the activity of a HesB/YadR/YfhF family protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of bis-glyceryl phosphate (polar fraction), which comprises
(a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of triacylglycerol, which comprises
(a) increasing or generating the activity of a threonine synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of glycerol phosphate (lipid fraction), which comprises
(a) increasing or generating the activity of a b0050-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of bis-glyceryl phosphate (polar fraction), which comprises
(a) increasing or generating the activity of a serine protease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of triacylglycerol, which comprises
(a) increasing or generating the activity of a serine protease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of bis-glyceryl phosphate (polar fraction), which comprises
(a) increasing or generating the activity of a ATP-binding component of a transport system non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating the activity of a b0518-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of bis-glyceryl phosphate (polar fraction), which comprises
(a) increasing or generating the activity of a phosphoglyceromutase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (polar fraction), which comprises
(a) increasing or generating the activity of a putrescine transport system permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

A further embodiment of the present invention relates to a process for the production of glycerol (lipid fraction), which comprises
(a) increasing or generating the activity of a methylglyoxal synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol (polar fraction), which comprises
(a) increasing or generating the activity of a methylglyoxal synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a b0970-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a b1003-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a 3-oxoacyl-[acyl-carrier-protein] synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating the activity of a sodium/proton antiporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of triacylglycerol, which comprises
(a) increasing or generating the activity of a b1234-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of glycerol (lipid fraction), which comprises
(a) increasing or generating the activity of a cardiolipin synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol phosphate (lipid fraction), which comprises
(a) increasing or generating the activity of a b1330-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of bis-glyceryl phosphate (polar fraction), which comprises
(a) increasing or generating the activity of a ATP-dependent RNA helicase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a phenylacetic acid degradation protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a lipoprotein precursor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a b1522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a lysozyme non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a b1556-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of triacylglycerol, which comprises
(a) increasing or generating the activity of a b1583-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of bis-glyceryl phosphate (polar fraction), which comprises
(a) increasing or generating the activity of a Fe—S subunit of oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating the activity of a acid shock protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of bis-glyceryl phosphate (polar fraction), which comprises
(a) increasing or generating the activity of a b1670-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a b1672-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of triacylglycerol, which comprises
(a) increasing or generating the activity of a b1703-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of bis-glyceryl phosphate (polar fraction), which comprises
(a) increasing or generating the activity of a phosphotransferase system component non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of triacylglycerol, which comprises
(a) increasing or generating the activity of a B1795-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of glycerol-3-phosphate (polar fraction), which comprises
(a) increasing or generating the activity of a L-serine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol-3-phosphate (polar fraction) or a composition comprising glycerol-3-phosphate (polar fraction) in said non-human organism or in the culture medium surrounding said non-human organism.

A further embodiment of the present invention relates to a process for the production of glycerol phosphate (lipid fraction), which comprises
(a) increasing or generating the activity of a b1837-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of triacylglycerol, which comprises
(a) increasing or generating the activity of a trehalose-phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of glycerol (lipid fraction), which comprises
(a) increasing or generating the activity of a imidazole glycerol phosphate synthase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol phosphate (lipid fraction), which comprises
(a) increasing or generating the activity of a b2032-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol (lipid fraction), which comprises
(a) increasing or generating the activity of a b2099-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of bis-glyceryl phosphate (polar fraction), which comprises
(a) increasing or generating the activity of a b2139-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating the activity of a ABC transporter permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol phosphate (lipid fraction), which comprises
(a) increasing or generating the activity of a ion-transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of bis-glyceryl phosphate (polar fraction), which comprises
(a) increasing or generating the activity of a b2399-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating the activity of a b2399-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol (lipid fraction), which comprises
(a) increasing or generating the activity of a ethanolamine utilization protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol phosphate (lipid fraction), which comprises
(a) increasing or generating the activity of a ethanolamine utilization protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol (lipid fraction), which comprises
(a) increasing or generating the activity of a ethanolamine utilization protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of bis-glyceryl phosphate (polar fraction), which comprises
(a) increasing or generating the activity of a b2474-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating the activity of a b2474-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol (lipid fraction), which comprises
(a) increasing or generating the activity of a putative transport system permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol phosphate (lipid fraction), which comprises
(a) increasing or generating the activity of a putative transport system permease protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of bis-glyceryl phosphate (polar fraction), which comprises
(a) increasing or generating the activity of a b2613-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (polar fraction), which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

A further embodiment of the present invention relates to a process for the production of glycerol (lipid fraction), which comprises
(a) increasing or generating the activity of a b2673-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol phosphate (lipid fraction), which comprises
(a) increasing or generating the activity of a b2673-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of bis-glyceryl phosphate (polar fraction), which comprises
(a) increasing or generating the activity of a murein transglycosylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of triacylglycerol, which comprises
(a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of glycerol (lipid fraction), which comprises
(a) increasing or generating the activity of a 2-C-methyl-D-erythritol 4-phosphate cytidylyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of triacylglycerol, which comprises
(a) increasing or generating the activity of a amino-acid acetyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of bis-glyceryl phosphate (polar fraction), which comprises
(a) increasing or generating the activity of a b3083-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a threonine/serine transporter non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a b3160-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a argininosuccinate synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol-3-phosphate (polar fraction), which comprises
(a) increasing or generating the activity of a acetyl CoA carboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol-3-phosphate (polar fraction) or a composition comprising glycerol-3-phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating the activity of a methyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of bis-glyceryl phosphate (polar fraction), which comprises
(a) increasing or generating the activity of a FKBP-type peptidyl-prolyl cis-trans isomerase (rotamase) non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a high-affinity branched-chain amino acid transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a b3777-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a b3791-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a enterobacterial common antigen polymerase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of triacylglycerol, which comprises
(a) increasing or generating the activity of a b3989-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of bis-glyceryl phosphate (polar fraction), which comprises
(a) increasing or generating the activity of a b4029-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of triacylglycerol, which comprises
(a) increasing or generating the activity of a b4050-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of bis-glyceryl phosphate (polar fraction), which comprises
(a) increasing or generating the activity of a lysyl-tRNA synthetase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol-3-phosphate (polar fraction), which comprises
(a) increasing or generating the activity of a purine-nucleoside phosphorylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol-3-phosphate (polar fraction) or a composition comprising glycerol-3-phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol phosphate (lipid fraction), which comprises
(a) increasing or generating the activity of a glutaredoxin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of bis-glyceryl phosphate (polar fraction), which comprises
(a) increasing or generating the activity of a GM02LC17485-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol phosphate (lipid fraction), which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol (polar fraction), which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

A further embodiment of the present invention relates to a process for the production of glycerol phosphate (lipid fraction), which comprises
(a) increasing or generating the activity of a cyclin D non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of bis-glyceryl phosphate (polar fraction), which comprises
(a) increasing or generating the activity of a nitrate/nitrite transport protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol phosphate (lipid fraction), which comprises
(a) increasing or generating the activity of a carbamoyl-phosphate synthase subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol (lipid fraction), which comprises
(a) increasing or generating the activity of a CDP-diacylglycerol-glycerol-3-phosphate 3-phosphatidyltransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of bis-glyceryl phosphate (polar fraction), which comprises
(a) increasing or generating the activity of a adenylate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating the activity of a acyl transferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol phosphate (lipid fraction), which comprises
(a) increasing or generating the activity of a coproporphyrinogen III oxidase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol (lipid fraction), which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol phosphate (lipid fraction), which comprises
(a) increasing or generating the activity of a oxidoreductase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol (polar fraction), which comprises
(a) increasing or generating the activity of a Mg-protoporphyrin IX monomethyl ester oxidative cyclase 66 kD subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a dihydrolipoamide dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol phosphate (lipid fraction), which comprises
(a) increasing or generating the activity of a aromatic acid decarboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol phosphate (lipid fraction), which comprises
(a) increasing or generating the activity of a phosphate import ATP-binding protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol phosphate (lipid fraction), which comprises
(a) increasing or generating the activity of a arginine decarboxylase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol phosphate (lipid fraction), which comprises
(a) increasing or generating the activity of a carbohydrate kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of bis-glyceryl phosphate (polar fraction), which comprises
(a) increasing or generating the activity of a precorrin-8X methylmutase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a NADH dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating the activity of a glutamate 5-kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol (lipid fraction), which comprises
(a) increasing or generating the activity of a threonine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol phosphate (lipid fraction), which comprises
(a) increasing or generating the activity of a threonine dehydratase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of bis-glyceryl phosphate (polar fraction), which comprises
(a) increasing or generating the activity of a photosystem I reaction centre subunit non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of triacylglycerol, which comprises
(a) increasing or generating the activity of a cell division protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of glycerol phosphate (lipid fraction), which comprises
(a) increasing or generating the activity of a metal-dependent hydrolase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol (lipid fraction), which comprises
(a) increasing or generating the activity of a TTC1386-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol-3-phosphate (polar fraction), which comprises
(a) increasing or generating the activity of a YAL013W-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol-3-phosphate (polar fraction) or a composition comprising glycerol-3-phosphate (polar fraction) in said non-human organism or in the culture medium surrounding said non-human organism.

A further embodiment of the present invention relates to a process for the production of glycerol (lipid fraction), which comprises
(a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol phosphate (lipid fraction), which comprises
(a) increasing or generating the activity of a acid phosphatase precursor non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol (lipid fraction), which comprises
(a) increasing or generating the activity of a cell division control protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol (polar fraction), which comprises
(a) increasing or generating the activity of a cell division control protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a ycr102c-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a ydr273w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol phosphate (lipid fraction), which comprises
(a) increasing or generating the activity of a molecular chaperone portein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of triacylglycerol, which comprises
(a) increasing or generating the activity of a YFL019C-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of bis-glyceryl phosphate (polar fraction), which comprises
(a) increasing or generating the activity of a alcohol dehydrogenase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating the activity of a biotin synthase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of triacylglycerol, which comprises
(a) increasing or generating the activity of a mitochondrial processing protease non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of bis-glyceryl phosphate (polar fraction), which comprises
(a) increasing or generating the activity of a YHR202W-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (polar fraction), which comprises
(a) increasing or generating the activity of a (DL)-glycerol-3-phosphatase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a YIL083C-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol phosphate (lipid fraction), which comprises
(a) increasing or generating the activity of a YJL062W-A-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of bis-glyceryl phosphate (polar fraction), which comprises
(a) increasing or generating the activity of a DnaJ-like chaperone non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a polygalacturonase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (polar fraction), which comprises
(a) increasing or generating the activity of a vacuolar sorting protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a YLR099C-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating the activity of a cyclin non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol (lipid fraction), which comprises
(a) increasing or generating the activity of a ylr326w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol phosphate (lipid fraction), which comprises
(a) increasing or generating the activity of a ylr326w-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol (lipid fraction), which comprises
(a) increasing or generating the activity of a heat shock protein Hsp40 non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol (lipid fraction), which comprises
(a) increasing or generating the activity of a phosphotransferase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol (lipid fraction), which comprises
(a) increasing or generating the activity of a Elongation factor Tu non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of bis-glyceryl phosphate (polar fraction), which comprises
(a) increasing or generating the activity of a YPL162C.protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating the activity of a mitogen-activated protein kinase non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of bis-glyceryl phosphate (polar fraction), which comprises
(a) increasing or generating the activity of a Zm_{—}4842_BE510522-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a 49747384_SOYBEAN-protein non-targeted in a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating the activity of a polypeptide comprising a polypeptide as depicted in the respective line in column 5 or 8 of Table II, application no. 19, or a homolog or a fragment thereof, a consensus sequence or at least one polypeptide motif as depicted in the respective line in column 8 of Table IV, application no. 19, whereby the respective line disclose in column 7 the fine chemical bis-glyceryl phosphate (polar fraction); or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical bis-glyceryl phosphate (polar fraction);
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of bis-glyceryl phosphate (polar fraction), or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating the activity of a polypeptide comprising a polypeptide as depicted in the respective line in column 5 or 8 of Table II, application no. 19, or a homolog or a fragment thereof, a consensus sequence or at least one polypeptide motif as depicted in the respective line in column 8 of Table IV, application no. 19, whereby the respective line disclose in column 7 the fine chemical glycerol (lipid fraction); or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical glycerol (lipid fraction);
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of glycerol (lipid fraction), or a composition comprising glycerol (lipid fraction) in said non-human organism or in the culture medium surrounding said non-human organism.

A further embodiment of the present invention relates to a process for the production of glycerol (polar fraction), which comprises
(a) increasing or generating the activity of a polypeptide comprising a polypeptide as depicted in the respective line in column 5 or 8 of Table II, application no. 19, or a homolog or a fragment thereof, a consensus sequence or at least one polypeptide motif as depicted in the respective line in column 8 of Table IV, application no. 19, whereby the respective line disclose in column 7 the fine chemical glycerol (polar fraction); or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical glycerol (polar fraction);
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of glycerol (polar fraction), or a composition comprising glycerol (polar fraction) in said non-human organism or in the culture medium surrounding said non-human organism.

A further embodiment of the present invention relates to a process for the production of glycerol phosphate (lipid fraction), which comprises
(a) increasing or generating the activity of a polypeptide comprising a polypeptide as depicted in the respective line in column 5 or 8 of Table II, application no. 19, or a homolog or a fragment thereof, a consensus sequence or at least one polypeptide motif as depicted in the respective line in column 8 of Table IV, application no. 19, whereby the respective line disclose in column 7 the fine chemical glycerol phosphate (lipid fraction); or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical glycerol phosphate (lipid fraction);
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of glycerol phosphate (lipid fraction), or a composition comprising glycerol phosphate (lipid fraction) in said non-human organism or in the culture medium surrounding said non-human organism.

A further embodiment of the present invention relates to a process for the production of glycerol-3-phosphate (polar fraction), which comprises
(a) increasing or generating the activity of a polypeptide comprising a polypeptide as depicted in the respective line in column 5 or 8 of Table II, application no. 19, or a homolog or a fragment thereof, a consensus sequence or at least one polypeptide motif as depicted in the respective line in column 8 of Table IV, application no. 19, whereby the respective line disclose in column 7 the fine chemical glycerol-3-phosphate (polar fraction); or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical glycerol-3-phosphate (polar fraction);
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of glycerol-3-phosphate (polar fraction), or a composition comprising glycerol-3-phosphate (polar fraction) in said non-human organism or in the culture medium surrounding said non-human organism

A further embodiment of the present invention relates to a process for the production of triacylglycerol, which comprises
(a) increasing or generating the activity of a polypeptide comprising a polypeptide as depicted in the respective line in column 5 or 8 of Table II, application no. 19, or a homolog or a fragment thereof, a consensus sequence or at least one polypeptide motif as depicted in the respective line in column 8 of Table IV, application no. 19, whereby the respective line disclose in column 7 the fine chemical triacylglycerol; or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical triacylglycerol;
non-targeted in a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of triacylglycerol, or a composition comprising triacylglycerol in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of At1g23110-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of eukaryotic translation initiation factor 5 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of eukaryotic translation initiation factor 5 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of triacylglycerol, which comprises
(a) increasing or generating one or more activities selected from the group consisting of At1g67340-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of glycerol (lipid fraction), which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of CBL-interacting protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of phosphatidylinositol 3- and 4-kinase family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of triacylglycerol, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of glycerol (lipid fraction), which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of triacylglycerol, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of glycerol (lipid fraction), which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of triacylglycerol, which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of calcium-dependent protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of triacylglycerol, which comprises
(a) increasing or generating one or more activities selected from the group consisting of heat shock transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of glycerol (lipid fraction), which comprises
(a) increasing or generating one or more activities selected from the group consisting of monothiol glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of protein phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of triacylglycerol, which comprises
(a) increasing or generating one or more activities selected from the group consisting of Glycine cleavage system T aminomethyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of sec-independent protein translocase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of transcriptional regulator protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of beta-hydroxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of triacylglycerol, which comprises
(a) increasing or generating one or more activities selected from the group consisting of beta-hydroxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of ThiF family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of recombinase A in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of NADH-quinone oxidoreductase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of triacylglycerol, which comprises
(a) increasing or generating one or more activities selected from the group consisting of hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of HesB/YadR/YfhF family protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of triacylglycerol, which comprises
(a) increasing or generating one or more activities selected from the group consisting of threonine synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of b0050-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of serine protease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of triacylglycerol, which comprises
(a) increasing or generating one or more activities selected from the group consisting of serine protease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of ATP-binding component of a transport system in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of b0518-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of phosphoglyceromutase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (polar fraction), which comprises
(a) increasing or generating one or more activities selected from the group consisting of putrescine transport system permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of methylglyoxal synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol (polar fraction), which comprises
(a) increasing or generating one or more activities selected from the group consisting of methylglyoxal synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of b0970-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of b1003-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of 3-oxoacyl-[acyl-carrier-protein] synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of sodium/proton antiporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of triacylglycerol, which comprises
(a) increasing or generating one or more activities selected from the group consisting of b1234-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of glycerol (lipid fraction), which comprises
(a) increasing or generating one or more activities selected from the group consisting of cardiolipin synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of b1330-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of ATP-dependent RNA helicase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of phenylacetic acid degradation protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of lipoprotein precursor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of b1522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of lysozyme in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of b1556-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of triacylglycerol, which comprises
(a) increasing or generating one or more activities selected from the group consisting of b1583-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of Fe—S subunit of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of acid shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of b1670-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of b1672-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of triacylglycerol, which comprises
(a) increasing or generating one or more activities selected from the group consisting of b1703-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of phosphotransferase system component in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of triacylglycerol, which comprises
(a) increasing or generating one or more activities selected from the group consisting of B1795-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of glycerol-3-phosphate (polar fraction), which comprises
(a) increasing or generating one or more activities selected from the group consisting of L-serine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol-3-phosphate (polar fraction) or a composition comprising glycerol-3-phosphate (polar fraction) in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of b1837-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of triacylglycerol, which comprises
(a) increasing or generating one or more activities selected from the group consisting of trehalose-phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of glycerol (lipid fraction), which comprises
(a) increasing or generating one or more activities selected from the group consisting of imidazole glycerol phosphate synthase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of b2032-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of b2099-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of b2139-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of ABC transporter permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of ion-transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of b2399-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of b2399-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of ethanolamine utilization protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of ethanolamine utilization protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of ethanolamine utilization protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of b2474-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of b2474-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of putative transport system permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of putative transport system permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of b2613-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (polar fraction), which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of b2673-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of b2673-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of murein transglycosylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of triacylglycerol, which comprises
(a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of glycerol (lipid fraction), which comprises
(a) increasing or generating one or more activities selected from the group consisting of 2-C-methyl-D-erythritol 4-phosphate cytidylyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of triacylglycerol, which comprises
(a) increasing or generating one or more activities selected from the group consisting of amino-acid acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of b3083-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of threonine/serine transporter in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of b3160-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of argininosuccinate synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol-3-phosphate (polar fraction), which comprises
(a) increasing or generating one or more activities selected from the group consisting of acetyl CoA carboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol-3-phosphate (polar fraction) or a composition comprising glycerol-3-phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of FKBP-type peptidyl-prolyl cis-trans isomerase (rotamase) in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of high-affinity branched-chain amino acid transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of b3777-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of b3791-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of enterobacterial common antigen polymerase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of triacylglycerol, which comprises
(a) increasing or generating one or more activities selected from the group consisting of b3989-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of b4029-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of triacylglycerol, which comprises
(a) increasing or generating one or more activities selected from the group consisting of b4050-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of lysyl-tRNA synthetase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol-3-phosphate (polar fraction), which comprises
(a) increasing or generating one or more activities selected from the group consisting of purine-nucleoside phosphorylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol-3-phosphate (polar fraction) or a composition comprising glycerol-3-phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of glutaredoxin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of GM02LC17485-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol (polar fraction), which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of nitrate/nitrite transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of carbamoyl-phosphate synthase subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of CDP-diacylglycerol-glycerol-3-phosphate 3-phosphatidyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of adenylate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of acyl transferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of coproporphyrinogen III oxidase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of oxidoreductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol (polar fraction), which comprises
(a) increasing or generating one or more activities selected from the group consisting of Mg-protoporphyrin IX monomethyl ester oxidative cyclase 66 kD subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of dihydrolipoamide dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of aromatic acid decarboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of phosphate import ATP-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of arginine decarboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of carbohydrate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of precorrin-8X methylmutase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of NADH dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of glutamate 5-kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of threonine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of threonine dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of photosystem I reaction centre subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of triacylglycerol, which comprises
(a) increasing or generating one or more activities selected from the group consisting of cell division protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of metal-dependent hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of TTC1386-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol-3-phosphate (polar fraction), which comprises
(a) increasing or generating one or more activities selected from the group consisting of YAL013W-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol-3-phosphate (polar fraction) or a composition comprising glycerol-3-phosphate (polar fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of acid phosphatase precursor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of cell division control protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol (polar fraction), which comprises
(a) increasing or generating one or more activities selected from the group consisting of cell division control protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of ycr102c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of ydr273w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of molecular chaperone portein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of triacylglycerol, which comprises
(a) increasing or generating one or more activities selected from the group consisting of YFL019C-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of alcohol dehydrogenase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of biotin synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of triacylglycerol, which comprises
(a) increasing or generating one or more activities selected from the group consisting of mitochondrial processing protease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of YHR202W-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (polar fraction), which comprises
(a) increasing or generating one or more activities selected from the group consisting of (DL)-glycerol-3-phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of YIL083C-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of YJL062W-A-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of DnaJ-like chaperone in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of polygalacturonase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (polar fraction), which comprises
(a) increasing or generating one or more activities selected from the group consisting of vacuolar sorting protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of YLR099C-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of cyclin in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of ylr326w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol phosphate (lipid fraction), which comprises
(a) increasing or generating one or more activities selected from the group consisting of ylr326w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of heat shock protein Hsp40 in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of phosphotransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of Elongation factor Tu in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of YPL162C.protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a) increasing or generating one or more activities selected from the group consisting of mitogen-activated protein kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of Zm_{—}4842_BE510522-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a) increasing or generating one or more activities selected from the group consisting of 49747384_SOYBEAN-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

A further embodiment of the present invention relates to a process for the production of bis-glyceryl phosphate (polar fraction), which comprises
(a1) increasing or generating the activity of a polypeptide comprising a polypeptide as depicted in the respective line in column 5 or 8 of Table II, application no. 19, or a homolog or fragment thereof, a consensus sequence or at least one polypeptide motif as depicted in column 8 of Table IV, application no. 19, whereby the respective line disclose in column 7 the fine chemical bis-glyceryl phosphate (polar fraction); or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical bis-glyceryl phosphate (polar fraction);
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 19, or a homolog or a fragment thereof, a consensus sequence or at least one polypeptide motif as depicted in the respective line in column 8 of Table IV, application no. 19, which is joined to a transit peptide, whereby the respective line disclose in column 7 the fine chemical bis-glyceryl phosphate (polar fraction); or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical bis-glyceryl phosphate (polar fraction);
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 19, or a homolog or a fragment thereof, a consensus sequence or at least one polypeptide motif as depicted in the respective line in column 8 of Table IV, application no. 19, whereby the respective line disclose in column 7 the fine chemical bis-glyceryl phosphate (polar fraction); or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical bis-glyceryl phosphate (polar fraction);
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof; and
(b) growing the non-human organism under conditions which permit the production of bis-glyceryl phosphate (polar fraction), or a composition comprising bis-glyceryl phosphate (polar fraction) in said non-human organism or in the culture medium surrounding said non-human organism.

A further embodiment of the present invention relates to a process for the production of glycerol (lipid fraction), which comprises
(a1) increasing or generating the activity of a polypeptide comprising a polypeptide as depicted in the respective line in column 5 or 8 of Table II, application no. 19, or a homolog or fragment thereof, a consensus sequence or at least one polypeptide motif as depicted in column 8 of Table IV, application no. 19, whereby the respective line disclose in column 7 the fine chemical glycerol (lipid fraction); or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical glycerol (lipid fraction);
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 19, or a homolog or a fragment thereof, a consensus sequence or at least one polypeptide motif as depicted in the respective line in column 8 of Table IV, application no. 19, which is joined to a transit peptide, whereby the respective line disclose in column 7 the fine chemical glycerol (lipid fraction); or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical glycerol (lipid fraction);
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 19, or a homolog or a fragment thereof, a consensus sequence or at least one polypeptide motif as depicted in the respective line in column 8 of Table IV, application no. 19, whereby the respective line disclose in column 7 the fine chemical glycerol (lipid fraction); or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical glycerol (lipid fraction);
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof; and
(b) growing the non-human organism under conditions which permit the production of glycerol (lipid fraction), or a composition comprising glycerol (lipid fraction) in said non-human organism or in the culture medium surrounding said non-human organism.

A further embodiment of the present invention relates to a process for the production of glycerol (polar fraction), which comprises
(a1) increasing or generating the activity of a polypeptide comprising a polypeptide as depicted in the respective line in column 5 or 8 of Table II, application no. 19, or a homolog or fragment thereof, a consensus sequence or at least one polypeptide motif as depicted in column 8 of Table IV, application no. 19, whereby the respective line disclose in column 7 the fine chemical glycerol (polar fraction); or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical glycerol (polar fraction);
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 19, or a homolog or a fragment thereof, a consensus sequence or at least one polypeptide motif as depicted in the respective line in column 8 of Table IV, application no. 19, which is joined to a transit peptide, whereby the respective line disclose in column 7 the fine chemical glycerol (polar fraction); or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical glycerol (polar fraction);
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 19, or a homolog or a fragment thereof, a consensus sequence or at least one polypeptide motif as depicted in the respective line in column 8 of Table IV, application no. 19, whereby the respective line disclose in column 7 the fine chemical glycerol (polar fraction); or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical glycerol (polar fraction);
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof; and
(b) growing the non-human organism under conditions which permit the production of glycerol (polar fraction), or a composition comprising glycerol (polar fraction) in said non-human organism or in the culture medium surrounding said non-human organism.

A further embodiment of the present invention relates to a process for the production of glycerol phosphate (lipid fraction), which comprises
(a1) increasing or generating the activity of a polypeptide comprising a polypeptide as depicted in the respective line in column 5 or 8 of Table II, application no. 19, or a homolog or fragment thereof, a consensus sequence or at least one polypeptide motif as depicted in column 8 of Table IV, application no. 19, whereby the respective line disclose in column 7 the fine chemical glycerol phosphate (lipid fraction); or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical glycerol phosphate (lipid fraction);
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 19, or a homolog or a fragment thereof, a consensus sequence or at least one polypeptide motif as depicted in the respective line in column 8 of Table IV, application no. 19, which is joined to a transit peptide, whereby the respective line disclose in column 7 the fine chemical glycerol phosphate (lipid fraction); or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical glycerol phosphate (lipid fraction);
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 19, or a homolog or a fragment thereof, a consensus sequence or at least one polypeptide motif as depicted in the respective line in column 8 of Table IV, application no. 19, whereby the respective line disclose in column 7 the fine chemical glycerol phosphate (lipid fraction); or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical glycerol phosphate (lipid fraction);
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof; and
(b) growing the non-human organism under conditions which permit the production of glycerol phosphate (lipid fraction), or a composition comprising glycerol phosphate (lipid fraction) in said non-human organism or in the culture medium surrounding said non-human organism.

A further embodiment of the present invention relates to a process for the production of glycerol-3-phosphate (polar fraction), which comprises
(a1) increasing or generating the activity of a polypeptide comprising a polypeptide as depicted in the respective line in column 5 or 8 of Table II, application no. 19, or a homolog or fragment thereof, a consensus sequence or at least one polypeptide motif as depicted in column 8 of Table IV, application no. 19, whereby the respective line disclose in column 7 the fine chemical glycerol-3-phosphate (polar fraction); or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical glycerol-3-phosphate (polar fraction);
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 19, or a homolog or a fragment thereof, a consensus sequence or at least one polypeptide motif as depicted in the respective line in column 8 of Table IV, application no. 19, which is joined to a transit peptide, whereby the respective line disclose in column 7 the fine chemical glycerol-3-phosphate (polar fraction); or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical glycerol-3-phosphate (polar fraction);
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 19, or a homolog or a fragment thereof, a consensus sequence or at least one polypeptide motif as depicted in the respective line in column 8 of Table IV, application no. 19, whereby the respective line disclose in column 7 the fine chemical glycerol-3-phosphate (polar fraction); or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical glycerol-3-phosphate (polar fraction);
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof; and
(b) growing the non-human organism under conditions which permit the production of glycerol-3-phosphate (polar fraction), or a composition comprising glycerol-3-phosphate (polar fraction) in said non-human organism or in the culture medium surrounding said non-human organism.

A further embodiment of the present invention relates to a process for the production of triacylglycerol, which comprises
(a1) increasing or generating the activity of a polypeptide comprising a polypeptide as depicted in the respective line in column 5 or 8 of Table II, application no. 19, or a homolog or fragment thereof, a consensus sequence or at least one polypeptide motif as depicted in column 8 of Table IV, application no. 19, whereby the respective line disclose in column 7 the fine chemical triacylglycerol; or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical triacylglycerol;
in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
(a2) increasing or generating the activity of a polypeptide comprising a polypeptide as depicted in the respective line in column 5 or 8 of Table II, application no. 19, or a homolog or a fragment thereof, a consensus sequence or at least one polypeptide motif as depicted in the respective line in column 8 of Table IV, application no. 19, which is joined to a transit peptide, whereby the respective line disclose in column 7 the fine chemical triacylglycerol; or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical triacylglycerol;
in a non-human organism or a part thereof; preferably a microorganism, a plant cell, a plant or a part thereof, as compared to a corresponding non-transformed wild type non-human organism or a part thereof; or
(a3) increasing or generating the activity of a polypeptide comprising a polypeptide as depicted in the respective line in column 5 or 8 of Table II, application no. 19, or a homolog or a fragment thereof, a consensus sequence or at least one polypeptide motif as depicted in the respective line in column 8 of Table IV, application no. 19, whereby the respective line disclose in column 7 the fine chemical triacylglycerol; or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical triacylglycerol;
in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof; and
(b) growing the non-human organism under conditions which permit the production of triacylglycerol, or a composition comprising triacylglycerol in said non-human organism or in the culture medium surrounding said non-human organism.

Accordingly, the present invention relates to a process for the production of glycerol phosphate (lipid fraction), which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of bis-glyceryl phosphate (polar fraction), which comprises
(a) increasing or generating the activity of a At1g23110-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a eukaryotic translation initiation factor 5 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol phosphate (lipid fraction), which comprises
(a) increasing or generating the activity of a eukaryotic translation initiation factor 5 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of triacylglycerol, which comprises
(a) increasing or generating the activity of a At1g67340-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of glycerol (lipid fraction), which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of bis-glyceryl phosphate (polar fraction), which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol phosphate (lipid fraction), which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of bis-glyceryl phosphate (polar fraction), which comprises
(a) increasing or generating the activity of a CBL-interacting protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a phosphatidylinositol 3- and 4-kinase family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of triacylglycerol, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of glycerol (lipid fraction), which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol (lipid fraction), which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of triacylglycerol, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of glycerol (lipid fraction), which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of triacylglycerol, which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of glycerol phosphate (lipid fraction), which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of bis-glyceryl phosphate (polar fraction), which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a calcium-dependent protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of triacylglycerol, which comprises
(a) increasing or generating the activity of a heat shock transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of glycerol (lipid fraction), which comprises
(a) increasing or generating the activity of a monothiol glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol phosphate (lipid fraction), which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol phosphate (lipid fraction), which comprises
(a) increasing or generating the activity of a protein phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol phosphate (lipid fraction), which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of triacylglycerol, which comprises
(a) increasing or generating the activity of a Glycine cleavage system T aminomethyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of glycerol phosphate (lipid fraction), which comprises
(a) increasing or generating the activity of a sec-independent protein translocase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol (lipid fraction), which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of bis-glyceryl phosphate (polar fraction), which comprises
(a) increasing or generating the activity of a transcriptional regulator protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a beta-hydroxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of triacylglycerol, which comprises
(a) increasing or generating the activity of a beta-hydroxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of bis-glyceryl phosphate (polar fraction), which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol phosphate (lipid fraction), which comprises
(a) increasing or generating the activity of a ThiF family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol (lipid fraction), which comprises
(a) increasing or generating the activity of a recombinase A in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of bis-glyceryl phosphate (polar fraction), which comprises
(a) increasing or generating the activity of a NADH-quinone oxidoreductase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of triacylglycerol, which comprises
(a) increasing or generating the activity of a hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of glycerol phosphate (lipid fraction), which comprises
(a) increasing or generating the activity of a HesB/YadR/YfhF family protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of bis-glyceryl phosphate (polar fraction), which comprises
(a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of triacylglycerol, which comprises
(a) increasing or generating the activity of a threonine synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of glycerol phosphate (lipid fraction), which comprises
(a) increasing or generating the activity of a b0050-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of bis-glyceryl phosphate (polar fraction), which comprises
(a) increasing or generating the activity of a serine protease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of triacylglycerol, which comprises
(a) increasing or generating the activity of a serine protease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of bis-glyceryl phosphate (polar fraction), which comprises
(a) increasing or generating the activity of a ATP-binding component of a transport system in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating the activity of a b0518-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of bis-glyceryl phosphate (polar fraction), which comprises
(a) increasing or generating the activity of a phosphoglyceromutase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (polar fraction), which comprises
(a) increasing or generating the activity of a putrescine transport system permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

Accordingly, the present invention relates to a process for the production of glycerol (lipid fraction), which comprises
(a) increasing or generating the activity of a methylglyoxal synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol (polar fraction), which comprises
(a) increasing or generating the activity of a methylglyoxal synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a b0970-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a b1003-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a 3-oxoacyl-[acyl-carrier-protein] synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating the activity of a sodium/proton antiporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of triacylglycerol, which comprises
(a) increasing or generating the activity of a b1234-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of glycerol (lipid fraction), which comprises
(a) increasing or generating the activity of a cardiolipin synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol phosphate (lipid fraction), which comprises
(a) increasing or generating the activity of a b1330-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of bis-glyceryl phosphate (polar fraction), which comprises
(a) increasing or generating the activity of a ATP-dependent RNA helicase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a phenylacetic acid degradation protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a lipoprotein precursor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a b1522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a lysozyme in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a b1556-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of triacylglycerol, which comprises
(a) increasing or generating the activity of a b1583-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of bis-glyceryl phosphate (polar fraction), which comprises
(a) increasing or generating the activity of a Fe—S subunit of oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating the activity of a acid shock protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of bis-glyceryl phosphate (polar fraction), which comprises
(a) increasing or generating the activity of a b1670-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a b1672-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of triacylglycerol, which comprises
(a) increasing or generating the activity of a b1703-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of bis-glyceryl phosphate (polar fraction), which comprises
(a) increasing or generating the activity of a phosphotransferase system component in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of triacylglycerol, which comprises
(a) increasing or generating the activity of a B1795-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of glycerol-3-phosphate (polar fraction), which comprises
(a) increasing or generating the activity of a L-serine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol-3-phosphate (polar fraction) or a composition comprising glycerol-3-phosphate (polar fraction) in said non-human organism or in the culture medium surrounding said non-human organism.

Accordingly, the present invention relates to a process for the production of glycerol phosphate (lipid fraction), which comprises
(a) increasing or generating the activity of a b1837-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of triacylglycerol, which comprises
(a) increasing or generating the activity of a trehalose-phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of glycerol (lipid fraction), which comprises
(a) increasing or generating the activity of a imidazole glycerol phosphate synthase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol phosphate (lipid fraction), which comprises
(a) increasing or generating the activity of a b2032-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol (lipid fraction), which comprises
(a) increasing or generating the activity of a b2099-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of bis-glyceryl phosphate (polar fraction), which comprises
(a) increasing or generating the activity of a b2139-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating the activity of a ABC transporter permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol phosphate (lipid fraction), which comprises
(a) increasing or generating the activity of a ion-transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of bis-glyceryl phosphate (polar fraction), which comprises
(a) increasing or generating the activity of a b2399-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating the activity of a b2399-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol (lipid fraction), which comprises
(a) increasing or generating the activity of a ethanolamine utilization protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol phosphate (lipid fraction), which comprises
(a) increasing or generating the activity of a ethanolamine utilization protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol (lipid fraction), which comprises
(a) increasing or generating the activity of a ethanolamine utilization protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of bis-glyceryl phosphate (polar fraction), which comprises
(a) increasing or generating the activity of a b2474-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating the activity of a b2474-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol (lipid fraction), which comprises
(a) increasing or generating the activity of a putative transport system permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol phosphate (lipid fraction), which comprises
(a) increasing or generating the activity of a putative transport system permease protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of bis-glyceryl phosphate (polar fraction), which comprises
(a) increasing or generating the activity of a b2613-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (polar fraction), which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

Accordingly, the present invention relates to a process for the production of glycerol (lipid fraction), which comprises
(a) increasing or generating the activity of a b2673-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol phosphate (lipid fraction), which comprises
(a) increasing or generating the activity of a b2673-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of bis-glyceryl phosphate (polar fraction), which comprises
(a) increasing or generating the activity of a murein transglycosylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of triacylglycerol, which comprises
(a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of glycerol (lipid fraction), which comprises
(a) increasing or generating the activity of a 2-C-methyl-D-erythritol 4-phosphate cytidylyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of triacylglycerol, which comprises
(a) increasing or generating the activity of a amino-acid acetyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of bis-glyceryl phosphate (polar fraction), which comprises
(a) increasing or generating the activity of a b3083-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a threonine/serine transporter in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a b3160-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a argininosuccinate synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol-3-phosphate (polar fraction), which comprises
(a) increasing or generating the activity of a acetyl CoA carboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol-3-phosphate (polar fraction) or a composition comprising glycerol-3-phosphate (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating the activity of a methyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of bis-glyceryl phosphate (polar fraction), which comprises
(a) increasing or generating the activity of a FKBP-type peptidyl-prolyl cis-trans isomerase (rotamase) in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a high-affinity branched-chain amino acid transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a b3777-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a b3791-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a enterobacterial common antigen polymerase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of triacylglycerol, which comprises
(a) increasing or generating the activity of a b3989-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of bis-glyceryl phosphate (polar fraction), which comprises
(a) increasing or generating the activity of a b4029-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of triacylglycerol, which comprises
(a) increasing or generating the activity of a b4050-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of bis-glyceryl phosphate (polar fraction), which comprises
(a) increasing or generating the activity of a lysyl-tRNA synthetase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol-3-phosphate (polar fraction), which comprises
(a) increasing or generating the activity of a purine-nucleoside phosphorylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol-3-phosphate (polar fraction) or a composition comprising glycerol-3-phosphate (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol phosphate (lipid fraction), which comprises
(a) increasing or generating the activity of a glutaredoxin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of bis-glyceryl phosphate (polar fraction), which comprises
(a) increasing or generating the activity of a GM02LC17485-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol phosphate (lipid fraction), which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol (polar fraction), which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

Accordingly, the present invention relates to a process for the production of glycerol phosphate (lipid fraction), which comprises
(a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of bis-glyceryl phosphate (polar fraction), which comprises
(a) increasing or generating the activity of a nitrate/nitrite transport protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol phosphate (lipid fraction), which comprises
(a) increasing or generating the activity of a carbamoyl-phosphate synthase subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol (lipid fraction), which comprises
(a) increasing or generating the activity of a CDP-diacylglycerol-glycerol-3-phosphate 3-phosphatidyltransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of bis-glyceryl phosphate (polar fraction), which comprises
(a) increasing or generating the activity of a adenylate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating the activity of a acyl transferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol phosphate (lipid fraction), which comprises
(a) increasing or generating the activity of a coproporphyrinogen III oxidase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol (lipid fraction), which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol phosphate (lipid fraction), which comprises
(a) increasing or generating the activity of a oxidoreductase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol (polar fraction), which comprises
(a) increasing or generating the activity of a Mg-protoporphyrin IX monomethyl ester oxidative cyclase 66 kD subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a dihydrolipoamide dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol phosphate (lipid fraction), which comprises
(a) increasing or generating the activity of a aromatic acid decarboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol phosphate (lipid fraction), which comprises
(a) increasing or generating the activity of a phosphate import ATP-binding protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol phosphate (lipid fraction), which comprises
(a) increasing or generating the activity of a arginine decarboxylase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol phosphate (lipid fraction), which comprises
(a) increasing or generating the activity of a carbohydrate kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of bis-glyceryl phosphate (polar fraction), which comprises
(a) increasing or generating the activity of a precorrin-8X methylmutase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a NADH dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating the activity of a glutamate 5-kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol (lipid fraction), which comprises
(a) increasing or generating the activity of a threonine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol phosphate (lipid fraction), which comprises
(a) increasing or generating the activity of a threonine dehydratase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of bis-glyceryl phosphate (polar fraction), which comprises
(a) increasing or generating the activity of a photosystem I reaction centre subunit in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of triacylglycerol, which comprises
(a) increasing or generating the activity of a cell division protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of glycerol phosphate (lipid fraction), which comprises
(a) increasing or generating the activity of a metal-dependent hydrolase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol (lipid fraction), which comprises
(a) increasing or generating the activity of a TTC1386-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol-3-phosphate (polar fraction), which comprises
(a) increasing or generating the activity of a YAL013W-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol-3-phosphate (polar fraction) or a composition comprising glycerol-3-phosphate (polar fraction) in said non-human organism or in the culture medium surrounding said non-human organism.

Accordingly, the present invention relates to a process for the production of glycerol (lipid fraction), which comprises
(a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol phosphate (lipid fraction), which comprises
(a) increasing or generating the activity of a acid phosphatase precursor in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol (lipid fraction), which comprises
(a) increasing or generating the activity of a cell division control protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol (polar fraction), which comprises
(a) increasing or generating the activity of a cell division control protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a ycr102c-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a ydr273w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol phosphate (lipid fraction), which comprises
(a) increasing or generating the activity of a molecular chaperone portein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of triacylglycerol, which comprises
(a) increasing or generating the activity of a YFL019C-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of bis-glyceryl phosphate (polar fraction), which comprises
(a) increasing or generating the activity of a alcohol dehydrogenase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating the activity of a biotin synthase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of triacylglycerol, which comprises
(a) increasing or generating the activity of a mitochondrial processing protease in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of triacylglycerol or a composition comprising triacylglycerol in said non-human organism or in the culture medium surrounding said non-human organism.

Accordingly, the present invention relates to a process for the production of bis-glyceryl phosphate (polar fraction), which comprises
(a) increasing or generating the activity of a YHR202W-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (polar fraction), which comprises
(a) increasing or generating the activity of a (DL)-glycerol-3-phosphatase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a YIL083C-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol phosphate (lipid fraction), which comprises
(a) increasing or generating the activity of a YJL062W-A-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of bis-glyceryl phosphate (polar fraction), which comprises
(a) increasing or generating the activity of a DnaJ-like chaperone in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a polygalacturonase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (polar fraction), which comprises
(a) increasing or generating the activity of a vacuolar sorting protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (polar fraction) or a composition comprising glycerol (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a YLR099C-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating the activity of a cyclin in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol (lipid fraction), which comprises
(a) increasing or generating the activity of a ylr326w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol phosphate (lipid fraction), which comprises
(a) increasing or generating the activity of a ylr326w-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol phosphate (lipid fraction) or a composition comprising glycerol phosphate (lipid fraction) in said non-human organism or in the culture medium surrounding said non-human organism.

Accordingly, the present invention relates to a process for the production of glycerol (lipid fraction), which comprises
(a) increasing or generating the activity of a heat shock protein Hsp40 in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol (lipid fraction), which comprises
(a) increasing or generating the activity of a phosphotransferase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of glycerol (lipid fraction), which comprises
(a) increasing or generating the activity of a Elongation factor Tu in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of bis-glyceryl phosphate (polar fraction), which comprises
(a) increasing or generating the activity of a YPL162C.protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating the activity of a mitogen-activated protein kinase in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of glycerol (lipid fraction) or a composition comprising glycerol (lipid fraction) in said non-human organism or in the culture medium surrounding said non-human organism.

Accordingly, the present invention relates to a process for the production of bis-glyceryl phosphate (polar fraction), which comprises
(a) increasing or generating the activity of a Zm_{—}4842_BE510522-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a 49747384_SOYBEAN-protein in the cytosol of a cell of a non-human organism or a part thereof, preferably a microorganism, a plant cell, a plant or a part thereof, as compared to a corresponding non-transformed wild type non-human organism or a part thereof; and
(b) growing the non-human organism or a part thereof under conditions which permit the production of bis-glyceryl phosphate (polar fraction) or a composition comprising bis-glyceryl phosphate (polar fraction) in said non-human organism or in the culture medium surrounding said non-human organism.

Accordingly, the present invention relates to a process for the production of bis-glyceryl phosphate (polar fraction), which comprises
(a) increasing or generating the activity of a polypeptide comprising a polypeptide as depicted in the respective line in column 5 or 8 of Table II, application no. 19, or a homolog or a fragment thereof, a consensus sequence or at least one polypeptide motif as depicted in the respective line in column 8 of Table IV, application no. 19, whereby the respective line disclose in column 7 the fine chemical bis-glyceryl phosphate (polar fraction); or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical bis-glyceryl phosphate (polar fraction);
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of bis-glyceryl phosphate (polar fraction), or a composition comprising bis-glyceryl phosphate (polar fraction) in said non-human organism or in the culture medium surrounding said non-human organism.

Accordingly, the present invention relates to a process for the production of glycerol (lipid fraction), which comprises
(a) increasing or generating the activity of a polypeptide comprising a polypeptide as depicted in the respective line in column 5 or 8 of Table II, application no. 19, or a homolog or a fragment thereof, a consensus sequence or at least one polypeptide motif as depicted in the respective line in column 8 of Table IV, application no. 19, whereby the respective line disclose in column 7 the fine chemical glycerol (lipid fraction); or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical glycerol (lipid fraction);
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of glycerol (lipid fraction), or a composition comprising glycerol (lipid fraction) in said non-human organism or in the culture medium surrounding said non-human organism.

Accordingly, the present invention relates to a process for the production of glycerol (polar fraction), which comprises
(a) increasing or generating the activity of a polypeptide comprising a polypeptide as depicted in the respective line in column 5 or 8 of Table II, application no. 19, or a homolog or a fragment thereof, a consensus sequence or at least one polypeptide motif as depicted in the respective line in column 8 of Table IV, application no. 19, whereby the respective line disclose in column 7 the fine chemical glycerol (polar fraction); or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical glycerol (polar fraction);
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of glycerol (polar fraction), or a composition comprising glycerol (polar fraction) in said non-human organism or in the culture medium surrounding said non-human organism.

Accordingly, the present invention relates to a process for the production of glycerol phosphate (lipid fraction), which comprises
(a) increasing or generating the activity of a polypeptide comprising a polypeptide as depicted in the respective line in column 5 or 8 of Table II, application no. 19, or a homolog or a fragment thereof, a consensus sequence or at least one polypeptide motif as depicted in the respective line in column 8 of Table IV, application no. 19, whereby the respective line disclose in column 7 the fine chemical glycerol phosphate (lipid fraction); or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical glycerol phosphate (lipid fraction);
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of glycerol phosphate (lipid fraction), or a composition comprising glycerol phosphate (lipid fraction) in said non-human organism or in the culture medium surrounding said non-human organism.

Accordingly, the present invention relates to a process for the production of glycerol-3-phosphate (polar fraction), which comprises
(a) increasing or generating the activity of a polypeptide comprising a polypeptide as depicted in the respective line in column 5 or 8 of Table II, application no. 19, or a homolog or a fragment thereof, a consensus sequence or at least one polypeptide motif as depicted in the respective line in column 8 of Table IV, application no. 19, whereby the respective line disclose in column 7 the fine chemical glycerol-3-phosphate (polar fraction); or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical glycerol-3-phosphate (polar fraction);
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of glycerol-3-phosphate (polar fraction), or a composition comprising glycerol-3-phosphate (polar fraction) in said non-human organism or in the culture medium surrounding said non-human organism

Accordingly, the present invention relates to a process for the production of triacylglycerol, which comprises
(a) increasing or generating the activity of a polypeptide comprising a polypeptide as depicted in the respective line in column 5 or 8 of Table II, application no. 19, or a homolog or a fragment thereof, a consensus sequence or at least one polypeptide motif as depicted in the respective line in column 8 of Table IV, application no. 19, whereby the respective line disclose in column 7 the fine chemical triacylglycerol; or
increasing or generating the activity of an expression product of one or more nucleic acid molecule(s) comprising a polynucleotide as depicted in the respective line in column 5 or 8 of Table I, application no. 19, preferably the coding region thereof, or a homolog or a fragment thereof, whereby the respective line disclose in column 7 the fine chemical triacylglycerol;
in the cytosol of a cell of a non-human organism or a part thereof; preferably a microorganism, a plant cell, a plant or a part thereof, as compared to a corresponding non-transformed wild type non-human organism or a part thereof; and
(b) growing the non-human organism under conditions which permit the production of triacylglycerol, or a composition comprising triacylglycerol in said non-human organism or in the culture medium surrounding said non-human organism.

In a further embodiment the activity of the polypeptide comprising a polypeptide as depicted in the respective line in column 5 or 8 of Table II, application no. 19, or a homolog or a fragment thereof, a consensus sequence or at least one polypeptide motif as depicted in the respective line in column 8 of Table IV, application no. 19, is increased or generated non-targeted in the above-mentioned process in a microorganism or plant or a part thereof.

In a further embodiment said polypeptide has the activity of the respective polypeptide represented by a protein comprising a polypeptide as depicted in the respective line in column 5 of Table II, application no. 19.

In a further embodiment the activity of the expression product of one or more nucleic acid molecule(s) comprising a polynucleotide as depicted in the respective line in column 5 or 8 of Table I, application no. 19, preferably the coding region thereof, or a homolog or a fragment thereof, is increased or generated non-targeted in the above-mentioned process in a microorganism or plant or a part thereof.

In a further embodiment the activity of the polypeptide comprising a polypeptide as depicted in the respective line in column 5 or 8 of Table II, application no. 19, or a homolog or a fragment thereof, a consensus sequence or at least one polypeptide motif as depicted in the respective line in column 8 of Table IV, application no. 19, is increased or generated in the above-mentioned process in an organelle preferably in plastids or mitochondria, especially in plastids, of a microorganism or plant.

In a further embodiment said polypeptide has the activity of the respective polypeptide represented by a protein comprising a polypeptide as depicted in the respective line in column 5 of Table II, application no. 19.

In a further embodiment the activity of the expression product of one or more nucleic acid molecule(s) comprising a polynucleotide as depicted in the respective line in column 5 or 8 of Table I, application no. 19, preferably the coding region thereof, or a homolog or a fragment thereof, is increased or generated in the above-mentioned process in an organelle, preferably in plasids or mitochondria, especially in plastids, of a microorganism or plant.

In a further embodiment the activity of the polypeptide comprising a polypeptide as depicted in the respective line in column 5 or 8 of Table II, application no. 19, or a homolog or a fragment thereof, a consensus sequence or at least one polypeptide motif as depicted in the respective line in column 8 of Table IV, application no. 19, is increased or generated in the above-mentioned process in the cyctosol of a cell, of a microorganism or plant.

In a further embodiment said polypeptide has the activity of the respective polypeptide represented by a protein comprising a polypeptide as depicted in the respective line in column 5 of Table II, application no. 19.

In a further embodiment the activity of the expression product of one or more nucleic acid molecule(s) comprising a polynucleotide as depicted in the respective line in column 5 or 8 of Table I, application no. 19, preferably the coding region thereof, or a homolog or a fragment thereof, is increased or generated in the above-mentioned process in the cytosol of a cell, of a microorganism or plant.

[0039.1.7.19] to [0066.1.7.19] for the disclosure of these paragraphs see [0039.1.7.7] to [0066.1.7.7] above

As mentioned above the nucleic acid sequences coding for the proteins as shown in Table II, application no. 19, column 5 or 8, or homologs or fragments thereof, are joined to a nucleic acid sequence encoding a plastidic transit peptide, e.g, if for the nucleic acid molecule in Table I, column 6 the term “plastidic” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide coding sequence is fused in frame to the nucleic acid sequence coding for proteins as shown in Table II, application no. 19, column 5 or 8, or homologs or fragments thereof, e.g., if for the nucleic acid molecule in Table I, column 6 the term “plastidic” is indicated.

[0068.1.7.19] to [0072.1.7.19] for the disclosure of these paragraphs see [0068.1.7.7] to [0072.1.7.7] above

As mentioned above the nucleic acid sequences coding for the proteins as shown in the respective line in Table II, application no. 19, column 5 or 8, or homologs or fragments thereof, are joined to a nucleic acid sequence encoding a mitochondric transit peptide, e.g., if for the respective nucleic acid molecule in Table I, column 6 the term “mitochondric” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in the respective line in Table II, application no. 19, column 5 or 8, or homologs or fragments thereof, e.g., if for the nucleic acid molecule in the respective line in Table I, column 6 the term “mitochondric” is indicated.

for the disclosure of this paragraph see [0074.1.7.7] above

for the disclosure of this paragraph see [0075.1.7.7] above

Nucleic acid sequences coding for the transit peptides may be chemically synthesized either in part or wholly according to structure of transit peptide sequences disclosed in the prior art. Said natural or chemically synthesized sequences can be directly linked to the sequences encoding the mature protein or via a linker nucleic acid sequence, which may be typically less than 500 base pairs, preferably less than 450, 400, 350, 300, 250 or 200 base pairs, more preferably less than 150, 100, 90, 80, 70, 60, 50, 40 or 30 base pairs and most preferably less than 25, 20, 15, 12, 9, 6 or 3 base pairs in length and are in frame to the coding sequence. Furthermore favorable nucleic acid sequences encoding transit peptides may comprise sequences derived from more than one biological and/or chemical source and may include a nucleic acid sequence derived from the amino-terminal region of the mature protein, which in its native state is linked to the transit peptide. In a preferred embodiment of the invention said amino-terminal region of the mature protein is typically less than 150 amino acids, preferably less than 140, 130, 120, 110, 100 or 90 amino acids, more preferably less than 80, 70, 60, 50, 40, 35, 30, 25 or 20 amino acids and most preferably less than 19, 18, 17, 16, 15, 14, 13, 12, 11 or 10 amino acids in length. But even shorter or longer stretches are also possible. In addition target sequences, which facilitate the transport of proteins to other cell compartments such as the vacuole, endoplasmic reticulum, golgi complex, glyoxysomes, peroxisomes, mitochondria (in case of a plastidal transit peptide) or plastids, especially chloroplasts (in case of a mitochondric transit peptide) may be also part of the inventive nucleic acid sequence. The proteins translated from said inventive nucleic acid sequences are a kind of fusion proteins that means the nucleic acid sequences encoding the plastidal transit peptide for example the ones shown in Table a, preferably the last one of the Table a are joint to the respective nucleic acid sequences shown in Table I, application no. 19, columns 5 or 8, especially the coding region thereof, or homologs or fragments thereof, in case of nucleic acid sequences being expressed in plastids, and that means the nucleic acid sequences encoding the mitochondric transit peptide for example the ones shown in Table b, preferably the last one of the Table b are joint to the respective nucleic acid sequences shown in Table I, application no. 19, columns 5 or 8, especially the coding region thereof, or homologs or fragments thereof, in case of nucleic acid sequences being expressed in mitochondria. The person skilled in the art is able to join said sequences in a functional manner. Advantageously the transit peptide part is cleaved off from the protein part shown in Table II, application no. 19, columns 5 or 8, or a homolog or fragment thereof, during or after the transport into the organelles, preferably into the plastids or mitochondria, respectively.

All products of the cleavage of the preferred transit peptide shown in the last line of Table a have preferably the N-terminal amino acid sequences QIA CSS or QIA EFQLTT in front of the start methionine of the respective protein mentioned in Table II, application no. 19, column 5 or 8. or homologs or fragments thereof. Other short amino acid sequences of an range of 1 to 20 amino acids preferable 2 to 15 amino acids, more preferable 3 to 10 amino acids most preferably 4 to 8 amino acids are also possible in front of the start methionine of the protein mentioned in Table II, application no. 19, columns 5 or 8, or homologs or fragments thereof. In case of the amino acid sequence QIA CSS the three amino acids in front of the start methionine are stemming from the LIC (=ligatation independent cloning) cassette. Said short amino acid sequence is preferred in the case of the expression of E. coli genes. In case of the amino acid sequence QIA EFQLTT the six amino acids in front of the start methionine are stemming from the LIC cassette. Said short amino acid sequence is preferred in the case of the expression of S. cerevisiae genes. The skilled worker knowns that other short sequences are also useful in the expression of the respective nucleic acid molecule comprising a polynucleotide as depicted in Table I, application no. 19, column 5 or 8, preferably the coding region thereof, or a homolog or a fragment thereof. Furthermore the skilled worker is aware of the fact that there is not a need for such short sequences in the expression of the genes.

for the disclosure of this paragraph see [0077.1.7.7] above

for the disclosure of this paragraph see [0078.1.7.7] above

Alternatively to the targeting of the respective sequences shown in Table II, application no. 19, columns 5 or 8, or homologs or fragments thereof, preferably of sequences in general encoded in the nucleus with the aid of the targeting sequences mentioned for example in Table a alone, Table b alone, or in combination with other targeting sequences preferably into plastids or mitochondria, respectively, the nucleic acids of the invention can directly be introduced into the plastidal or mitochondrial genome. Therefore in a preferred embodiment the respective nucleic acid sequences shown in the respective line in Table I, application no. 19, columns 5 or 8, preferably the coding region thereof, or homologs or fragments thereof, are directly introduced and expressed in plastids e.g., if for the nucleic acid molecule in the respective line in Table I, column 6, the term “plastidic” is indicated, or are directly introduced and expressed in mitochondria e.g., if for the nucleic acid molecule in the respective line in Table I, column 6 the term “mitochondric” is indicated. The term “introduced” in the context of this specification shall mean the insertion of a nucleic acid sequence into the organism by means of a “transfection”, “transduction” or preferably by “transformation”.

A plastid, such as a chloroplast, or a mitochondrion, has been “transformed” by an exogenous (preferably foreign) nucleic acid sequence if a nucleic acid sequence has been introduced into the plastid or mitochondrion that means that this sequence has crossed the membrane or the membranes of the plastid or of the mitochondrion. The exogenous, preferably foreign, DNA may be integrated (covalently linked) into plastid or mitochondrial DNA making up the genome of the plastid or mitochondrion, or it may remain unintegrated (e.g., by including a chloroplast origin of replication). “Stably” integrated DNA sequences are those, which are inherited through plastid or mitochondrion replication, thereby transferring new plastids or mitochondria, with the features of the integrated DNA sequence to the progeny.

[0080.1.7.19] to [0083.1.7.19] for the disclosure of these paragraphs see [0080.1.7.7] to [0083.1.7.7] above

For the process of the present invention it may be of great advantage that by transforming the plastids the intraspecies specific transgene flow is blocked, because a lot of species such as corn, cotton and rice have a strict maternal inheritance of plastids. By placing the respective genes specified in the respective line in Table I, application no. 19, columns 5 or 8, preferably the coding region, or homologs or fragments thereof, e.g. if for the nucleic acid molecule in the respective line in column 6 of Table I the term “plastidic” is indicated, in the plastids of plants, these genes will not be present in the pollen of said plants.

A further preferred embodiment of the invention relates to the use of so called “chloroplast localization sequences”, in which a first RNA sequence or molecule is capable of transporting or “chaperoning” a second RNA sequence, such as a RNA sequence transcribed from the sequences depicted in the respective line in Table I, application no. 19, columns 5 or 8, preferably the coding region thereof, or a homolog or a fragment thereof, or a sequence encoding a protein, as depicted in the respective line in Table II, application no. 19, columns 5 and 8, or a homolog or a fragment thereof, from an external environment inside a cell or outside a plastid into a chloroplast. In one embodiment the chloroplast localization signal is substantially similar or complementary to a complete or intact viroid sequence. The chloroplast localization signal may be encoded by a DNA sequence, which is transcribed into the chloroplast localization RNA. The term “viroid” refers to a naturally occurring single stranded RNA molecule (Flores, C. R. Acad. Sci. III. 324 (10), 943 (2001)). Viroids usually contain about 200-500 nucleotides and generally exist as circular molecules. Examples of viroids that contain chloroplast localization signals include but are not limited to ASBVd, PLMVd, CChMVd and ELVd. The viroid sequence or a functional part of it can be fused to the sequences depicted in the respective line in Table I, application no. 19, columns 5 or 8, preferably the coding region thereof, or a homolog or a fragment thereof, or a sequence encoding a protein, as depicted in the respective line in Table II, application no. 19, columns 5 or 8, or a homolog or a fragment thereof, in such a manner that the viroid sequence transports a sequence transcribed from a sequence as depicted in the respective line in Table I, application no. 19 columns 5 or 8, preferably the coding region thereof, or a homolog or a fragment thereof, or a sequence encoding a protein as depicted in the respective line in Table II, application no. 19 columns 5 or 8, or a homolog or a fragment thereof, into the chloroplasts, e.g. if for the nucleic acid molecule in column 6 of Table I the term “plastidic” is indicated. A preferred embodiment uses a modified ASBVd (Navarro et al., Virology. 268 (1), 218 (2000)).

In a further specific embodiment the protein to be expressed in the plastids such as the proteins depicted in the respective line in Table II, application no. 19, columns 5 or 8, or a homolog or a fragment thereof, e.g. if for the nucleic acid molecule in the respective line in column 6 of Table I the term “plastidic” is indicated, are encoded by different nucleic acids. Such a method is disclosed in WO 2004/040973, which shall be incorporated by reference. WO 2004/040973 teaches a method, which relates to the translocation of an RNA corresponding to a gene or gene fragment into the chloroplast by means of a chloroplast localization sequence. The genes, which should be expressed in the plant or plant cells, are split into nucleic acid fragments, which are introduced into different compartments in the plant e.g. the nucleus, the plastids and/or mitochondria. Additionally plant cells are described in which the chloroplast contains a ribozyme fused at one end to an RNA encoding a fragment of a protein used in the inventive process such that the ribozyme can trans-splice the translocated fusion RNA to the RNA encoding the gene fragment to form and as the case may be reunite the nucleic acid fragments to an intact mRNA encoding a functional protein for example as disclosed in the respective line in Table II, application no. 19, columns 5 or 8, or a homolog or a fragment thereof.

In a preferred embodiment of the invention the nucleic acid sequences as shown in the respective line in Table I, application no. 19, columns 5 or 8, preferably the coding region thereof, or homologs or fragments thereof, e.g. if for the nucleic acid molecule in the respective line in column 6 of Table I the term “plastidic” is indicated, used in the inventive process are transformed into plastids, which are metabolically active. Those plastids should preferably maintain at a high copy number in the plant or plant tissue of interest, most preferably the chloroplasts found in green plant tissues, such as leaves or cotyledons or in seeds.

For a good expression in the plastids the nucleic acid sequences as shown in the respective line in Table I, application no. 19, columns 5 or 8, preferably the coding region thereof, or homologs or fragments thereof, e.g. if for the nucleic acid molecule in the respective line in column 6 of Table I the term “plastidic” is indicated, are introduced into an expression cassette using preferably a promoter and terminater, which are active in plastids preferably a chloroplast promoter. Examples of such promoters include the psbA promoter from the gene from spinach or pea, the rbcL promoter, and the atpB promoter from corn.

[0089.1.7.19] to [0092.1.7.19] for the disclosure of these paragraphs see [0089.1.7.7] to [0092.1.7.7] above

Advantageously the process for the production of the fine chemical bis-glyceryl phosphate (polar fraction), glycerol (lipid fraction), glycerol (polar fraction), glycerol phosphate (lipid fraction), glycerol-3-phosphate (polar fraction), and/or triacylglycerol leads to an enhanced production of the respective fine chemical. The terms “enhanced” or “increase” mean at least a 10%, 20%, 30%, 40% or 50%, preferably at least 60%, 70%, 80%, 90% or 100%, more preferably 150%, 200%, 300%, 400% or 500% higher production of the respective fine chemical bis-glyceryl phosphate (polar fraction), glycerol (lipid fraction), glycerol (polar fraction), glycerol phosphate (lipid fraction), glycerol-3-phosphate (polar fraction), and/or triacylglycerol in comparison to the wild-type as defined above, e.g. that means in comparison to a non-human organism without the aforementioned modification of the activity of a protein as shown in the respective line in Table II, application no. 19, column 5 or 8, or a fragment or a homolog thereof. The modification of the activity of a protein as shown in the respective line in Table II, application no. 19, column 5 or 8, or a homolog or a fragment thereof, or their combination can be achieved by joining the protein to a respective transit peptide, e.g. if for the respective encoding nucleic acid molecule in column 6 of Table I the term “plastidic” or “mitochondric” is indicated.

Surprisingly it was found, that the transgenic expression of a protein as shown in the respective line in Table II, application no. 19, column 5, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, conferred a production of or an increase in bis-glyceryl phosphate (polar fraction), glycerol (lipid fraction), glycerol (polar fraction), glycerol phosphate (lipid fraction), glycerol-3-phosphate (polar fraction), and/or triacylglycerol,respectively, to the transgenic non-human organism as compared to a corresponding non-transformed wild type.

In one embodiment the transgenic expression of a protein as shown in the respective line in Table II, application no. 19, column 5 or 8, or a homolog or a fragment thereof, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, confers a production of or an increase in bis-glyceryl phosphate (polar fraction), glycerol (lipid fraction), glycerol (polar fraction), glycerol phosphate (lipid fraction), glycerol-3-phosphate (polar fraction), and/or triacylglycerol, respectively, to the transgenic non-human organism as compared to a corresponding non-transformed wild type.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a protein phosphatase, or if the activity of the polypeptide At1g07430, preferably represented by SEQ ID NO. 17452, or a homolog or fragment thereof, or if the activity of a polypeptide encoded by a nucleic acid molecule comprising the nucleic acid SEQ ID NO. 17451, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 17451 or polypeptide SEQ ID NO. 17452, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical glycerol phosphate (lipid fraction). For example, an increase of the glycerol phosphate (lipid fraction) of at least 1 percent, particularly in a range of 21 to 63-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a At1g23110-protein, or if the activity of the polypeptide At1g23110, preferably represented by SEQ ID NO. 102087, or a homolog or fragment thereof, or if the activity of a polypeptide encoded by a nucleic acid molecule comprising the nucleic acid SEQ ID NO. 102086, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 102086 or polypeptide SEQ ID NO. 102087, respectively, or a homolog or a fragment thereof, is increased or generated, or if the activity At1g23110-protein is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 264 to 530-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a eukaryotic translation initiation factor 5, or if the activity of the polypeptide At1g36730, preferably represented by SEQ ID NO. 17969, or a homolog or fragment thereof, or if the activity of a polypeptide encoded by a nucleic acid molecule comprising the nucleic acid SEQ ID NO. 17968, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 17968 or polypeptide SEQ ID NO. 17969, respectively, or a homolog or a fragment thereof, is increased or generated, or if the activity eukaryotic translation initiation factor 5 is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 38 to 72-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a eukaryotic translation initiation factor 5, or if the activity of the polypeptide At1g36730, preferably represented by SEQ ID NO. 17969, or a homolog or fragment thereof, or if the activity of a polypeptide encoded by a nucleic acid molecule comprising the nucleic acid SEQ ID NO. 17968, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 17968 or polypeptide SEQ ID NO. 17969, respectively, or a homolog or a fragment thereof, is increased or generated, or if the activity eukaryotic translation initiation factor 5 is increased or generated in a non-human organism, like a microorganism or a plant cell, plant or part thereof, especially with non-targeted localization, whereby the respective line disclose in column 7 the fine chemical glycerol phosphate (lipid fraction). For example, an increase of the glycerol phosphate (lipid fraction) of at least 1 percent, particularly in a range of 24 to 39-percent is conferred as compared to a corresponding non-transformed wild type non-human organism.

Accordingly, in one embodiment, an increase of triacylglycerol in a non-human organism, as compared to a corresponding non-transformed wild type non-human organism, is conferred in the process of the invention, if the activity of a polypeptide showing the activity of a At1g67340-protein, or if the activity of the polypeptide At1g67340, preferably represented by SEQ ID NO. 19365, or a homolog or fragment thereof, or if the activity of a polypeptide encoded by a nucleic acid molecule comprising the nucleic acid SEQ ID NO. 19364, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 19364 or polypeptide SEQ ID NO. 19365, respectively, or a homolog or a fragment thereof, is increased or generated, or if the activity At1g67340-protein is increased or generated in a non-human organism, like a microorganism or a plant cell, plant or part thereof, especially with non-targeted localization, whereby the respective line disclose in column 7 the fine chemical triacylglycerol. For example, an increase of the triacylglycerol of at least 1 percent, particularly in a range of 39 to 135-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a transcription factor, or if the activity of the polypeptide At1g68320, preferably represented by SEQ ID NO. 1062, or a homolog or fragment thereof, or if the activity of a polypeptide encoded by a nucleic acid molecule comprising the nucleic acid SEQ ID NO. 1061, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 1061 or polypeptide SEQ ID NO. 1062, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 16 to 57-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a protein phosphatase, or if the activity of the polypeptide At1g72770, preferably represented by SEQ ID NO. 19420, or a homolog or fragment thereof, or if the activity of a polypeptide encoded by a nucleic acid molecule comprising the nucleic acid SEQ ID NO. 19419, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 19419 or polypeptide SEQ ID NO. 19420, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 40 to 78-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a protein phosphatase, or if the activity of the polypeptide At1g72770, preferably represented by SEQ ID NO. 19420, or a homolog or fragment thereof, or if the activity of a polypeptide encoded by a nucleic acid molecule comprising the nucleic acid SEQ ID NO. 19419, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 19419 or polypeptide SEQ ID NO. 19420, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 23 to 67-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a protein phosphatase, or if the activity of the polypeptide At1g72770, preferably represented by SEQ ID NO. 19420, or a homolog or fragment thereof, or if the activity of a polypeptide encoded by a nucleic acid molecule comprising the nucleic acid SEQ ID NO. 19419, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 19419 or polypeptide SEQ ID NO. 19420, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical glycerol phosphate (lipid fraction). For example, an increase of the glycerol phosphate (lipid fraction) of at least 1 percent, particularly in a range of 17 to 56-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a CBL-interacting protein kinase, or if the activity of the polypeptide At2g30360, preferably represented by SEQ ID NO. 19920, or a homolog or fragment thereof, or if the activity of a polypeptide encoded by a nucleic acid molecule comprising the nucleic acid SEQ ID NO. 19919, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 19919 or polypeptide SEQ ID NO. 19920, respectively, or a homolog or a fragment thereof, is increased or generated, or if the activity CBL-interacting protein kinase is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 39 to 69-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a phosphatidylinositol 3- and 4-kinase family protein, or if the activity of the polypeptide At2g46500, preferably represented by SEQ ID NO. 21107, or a homolog or fragment thereof, or if the activity of a polypeptide encoded by a nucleic acid molecule comprising the nucleic acid SEQ ID NO. 21106, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 21106 or polypeptide SEQ ID NO. 21107, respectively, or a homolog or a fragment thereof, is increased or generated, or if the activity phosphatidylinositol 3- and 4-kinase family protein is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 45 to 68-percent is conferred as compared to a corresponding non-transformed wild type non-human organism.

Accordingly, in one embodiment, an increase of triacylglycerol in a non-human organism, as compared to a corresponding non-transformed wild type non-human organism, is conferred in the process of the invention, if the activity of a polypeptide showing the activity of a heat shock transcription factor, or if the activity of the polypeptide At3g02990, preferably represented by SEQ ID NO. 68658, or a homolog or fragment thereof, or if the activity of a polypeptide encoded by a nucleic acid molecule comprising the nucleic acid SEQ ID NO. 68657, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 68657 or polypeptide SEQ ID NO. 68658, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line disclose in column 7 the fine chemical triacylglycerol. For example, an increase of the triacylglycerol of at least 1 percent, particularly in a range of 39 to 91-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a transcription factor, or if the activity of the polypeptide At4g09960, preferably represented by SEQ ID NO. 109718, or a homolog or fragment thereof, or if the activity of a polypeptide encoded by a nucleic acid molecule comprising the nucleic acid SEQ ID NO. 109717, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 109717 or polypeptide SEQ ID NO. 109718, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, partitularly in a range of 18 to 25-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 16 to 85-percent is conferred as compared to a corresponding non-transformed wild type non-human organism.

Accordingly, in one embodiment, an increase of triacylglycerol in a non-human organism, as compared to a corresponding non-transformed wild type non-human organism, is conferred in the process of the invention, if the activity of a polypeptide showing the activity of a monothiol glutaredoxin, or if the activity of the polypeptide At4g15660, preferably represented by SEQ ID NO. 23483, or a homolog or fragment thereof, or if the activity of a polypeptide encoded by a nucleic acid molecule comprising the nucleic acid SEQ ID NO. 23482, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 23482 or polypeptide SEQ ID NO. 23483, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical triacylglycerol. For example, an increase of the triacylglycerol of at least 1 percent, particularly in a range of 33 to 158-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a monothiol glutaredoxin, or if the activity of the polypeptide At4g15670, preferably represented by SEQ ID NO. 3280, or a homolog or fragment thereof, or if the activity of a polypeptide encoded by a nucleic acid molecule comprising the nucleic acid SEQ ID NO. 3279, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 3279 or polypeptide SEQ ID NO. 3280, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 53 to 59-percent is conferred as compared to a corresponding non-transformed wild type non-human organism.

Accordingly, in one embodiment, an increase of triacylglycerol in a non-human organism, as compared to a corresponding non-transformed wild type non-human organism, is conferred in the process of the invention, if the activity of a polypeptide showing the activity of a monothiol glutaredoxin, or if the activity of the polypeptide At4g15670, preferably represented by SEQ ID NO. 3280, or a homolog or fragment thereof, or if the activity of a polypeptide encoded by a nucleic acid molecule comprising the nucleic acid SEQ ID NO. 3279, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 3279 or polypeptide SEQ ID NO. 3280, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical triacylglycerol. For example, an increase of the triacylglycerol of at least 1 percent, particularly in a range of 38 to 150-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a protein phosphatase, or if the activity of the polypeptide At4g26080, preferably represented by SEQ ID NO. 68850, or a homolog or fragment thereof, or if the activity of a polypeptide encoded by a nucleic acid molecule comprising the nucleic acid SEQ ID NO. 68849, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 68849 or polypeptide SEQ ID NO. 68850, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical glycerol phosphate (lipid fraction). For example, an increase of the glycerol phosphate (lipid fraction) of at least 1 percent, particularly in a range of 17 to 35-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a cyclin D , or if the activity of the polypeptide At4g34160, preferably represented by SEQ ID NO. 24312, or a homolog or fragment thereof, or if the activity of a polypeptide encoded by a nucleic acid molecule comprising the nucleic acid SEQ ID NO. 24311, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 24311 or polypeptide SEQ ID NO. 24312, respectively, or a homolog or a fragment thereof, is increased or generated, or if the activity cyclin D is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 50 to 76-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a calcium-dependent protein kinase, or if the activity of the polypeptide At4g35310, preferably represented by SEQ ID NO. 4349, or a homolog or fragment thereof, or if the activity of a polypeptide encoded by a nucleic acid molecule comprising the nucleic acid SEQ ID NO. 4348, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 4348 or polypeptide SEQ ID NO. 4349, respectively, or a homolog or a fragment thereof, is increased or generated, or if the activity calcium-dependent protein kinase is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 39 to 145-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a heat shock transcription factor, or if the activity of the polypeptide At5g03720, preferably represented by SEQ ID NO. 24439, or a homolog or fragment thereof, or if the activity of a polypeptide encoded by a nucleic acid molecule comprising the nucleic acid SEQ ID NO. 24438, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 24438 or polypeptide SEQ ID NO. 24439, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 20 to 29-percent is conferred as compared to a corresponding non-transformed wild type non-human organism.

Accordingly, in one embodiment, an increase of triacylglycerol in a non-human organism, as compared to a corresponding non-transformed wild type non-human organism, is conferred in the process of the invention, if the activity of a polypeptide showing the activity of a heat shock transcription factor, or if the activity of the polypeptide At5g03720, preferably represented by SEQ ID NO. 24439, or a homolog or fragment thereof, or if the activity of a polypeptide encoded by a nucleic acid molecule comprising the nucleic acid SEQ ID NO. 24438, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 24438 or polypeptide SEQ ID NO. 24439, respectively, or a homolog or a fragment thereof, is increased or generated, or if the activity heat shock transcription factor is increased or generated in a non-human organism, like a microorganism or a plant cell, plant or part thereof, especially with non-targeted localization, whereby the respective line disclose in column 7 the fine chemical triacylglycerol. For example, an increase of the triacylglycerol of at least 1 percent, particularly in a range of 37 to 99-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a monothiol glutaredoxin, or if the activity of the polypeptide At5g18600, preferably represented by SEQ ID NO. 4905, or a homolog or fragment thereof, or if the activity of a polypeptide encoded by a nucleic acid molecule comprising the nucleic acid SEQ ID NO. 4904, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 4904 or polypeptide SEQ ID NO. 4905, respectively, or a homolog or a fragment thereof, is increased or generated, or if the activity monothiol glutaredoxin is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 17 to 34-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a protein phosphatase, or if the activity of the polypeptide At5g57050, preferably represented by SEQ ID NO. 5319, or a homolog or fragment thereof, or if the activity of a polypeptide encoded by a nucleic acid molecule comprising the nucleic acid SEQ ID NO. 5318, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 5318 or polypeptide SEQ ID NO. 5319, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical glycerol phosphate (lipid fraction). For example, an increase of the glycerol phosphate (lipid fraction) of at least 1 percent, particularly in a range of 19 to 35-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a protein phosphatase, or if the activity of the polypeptide At5g59220, preferably represented by SEQ ID NO. 25499, or a homolog or fragment thereof, or if the activity of a polypeptide encoded by a nucleic acid molecule comprising the nucleic acid SEQ ID NO. 25498, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 25498 or polypeptide SEQ ID NO. 25499, respectively, or a homolog or a fragment thereof, is increased or generated, or if the activity protein phosphatase is increased or generated in a non-human organism, like a microorganism or a plant cell, plant or part thereof, especially with non-targeted localization, whereby the respective line disclose in column 7 the fine chemical glycerol phosphate (lipid fraction). For example, an increase of the glycerol phosphate (lipid fraction) of at least 1 percent, particularly in a range of 18 to 39-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a pyruvate kinase, or if the activity of the polypeptide At5g63680, preferably represented by SEQ ID NO. 70039, or a homolog or fragment thereof, or if the activity of a polypeptide encoded by a nucleic acid molecule comprising the nucleic acid SEQ ID NO. 70038, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 70038 or polypeptide SEQ ID NO. 70039, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line disclose in column 7 the fine chemical glycerol phosphate (lipid fraction). For example, an increase of the glycerol phosphate (lipid fraction) of at least 1 percent, particularly in a range of 19 to 47-percent is conferred as compared to a corresponding non-transformed wild type non-human organism.

Accordingly, in one embodiment, an increase of triacylglycerol in a non-human organism, as compared to a corresponding non-transformed wild type non-human organism, is conferred in the process of the invention, if the activity of a polypeptide showing the activity of a Glycine cleavage system T aminomethyltransferase, or if the activity of the polypeptide AVINDRAFT_{—}0539, preferably represented by SEQ ID NO. 70716, or a homolog or fragment thereof, or if the activity of a polypeptide encoded by a nucleic acid molecule comprising the nucleic acid SEQ ID NO. 70715, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 70715 or polypeptide SEQ ID NO. 70716, respectively, or a homolog or a fragment thereof, is increased or generated, or if the activity Glycine cleavage system T aminomethyltransferase is increased or generated in a non-human organism, like a microorganism or a plant cell, plant or part thereof, especially with non-targeted localization, whereby the respective line disclose in column 7 the fine chemical triacylglycerol. For example, an increase of the triacylglycerol of at least 1 percent, particularly in a range of 35 to 52-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a sec-independent protein translocase, or if the activity of the polypeptide AvinDRAFT_{—}1624, preferably represented by SEQ ID NO. 26197, or a homolog or fragment thereof, or if the activity of a polypeptide encoded by a nucleic acid molecule comprising the nucleic acid SEQ ID NO. 26196, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 26196 or polypeptide SEQ ID NO. 26197, respectively, or a homolog or a fragment thereof, is increased or generated, or if the activity sec-independent protein translocase is increased or generated in a non-human organism, like a microorganism or a plant cell, plant or part thereof, especially with non-targeted localization, whereby the respective line disclose in column 7 the fine chemical glycerol phosphate (lipid fraction). For example, an increase of the glycerol phosphate (lipid fraction) of at least 1 percent, particularly in a range of 19 to 29-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a oxidoreductase, or if the activity of the polypeptide AvinDRAFT_{—}1788, preferably represented by SEQ ID NO. 114084, or a homolog or fragment thereof, or if the activity of a polypeptide encoded by a nucleic acid molecule comprising the nucleic acid SEQ ID NO. 114083, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 114083 or polypeptide SEQ ID NO. 114084, respectively, or a homolog or a fragment thereof, is increased or generated, or if the activity oxidoreductase is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 19 to 29-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a transcriptional regulator protein, or if the activity of the polypeptide AvinDRAFT_{—}2010, preferably represented by SEQ ID NO. 114199, or a homolog or fragment thereof, or if the activity of a polypeptide encoded by a nucleic acid molecule comprising the nucleic acid SEQ ID NO. 114198, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 114198 or polypeptide SEQ ID NO. 114199, respectively, or a homolog or a fragment thereof, is increased or generated, or if the activity transcriptional regulator protein is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 38 to 64-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a beta-hydroxylase, or if the activity of the polypeptide AvinDRAFT_{—}2091, preferably represented by SEQ ID NO. 6041, or a homolog or fragment thereof, or if the activity of a polypeptide encoded by a nucleic acid molecule comprising the nucleic acid SEQ ID NO. 6040, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 6040 or polypeptide SEQ ID NO. 6041, respectively, or a homolog or a fragment thereof, is increased or generated, or if the activity beta-hydroxylase is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 39 to 71-percent is conferred as compared to a corresponding non-transformed wild type non-human organism.

Accordingly, in one embodiment, an increase of triacylglycerol in a non-human organism, as compared to a corresponding non-transformed wild type non-human organism, is conferred in the process of the invention, if the activity of a polypeptide showing the activity of a beta-hydroxylase, or if the activity of the polypeptide AvinDRAFT_{—}2091, preferably represented by SEQ ID NO. 6041, or a homolog or fragment thereof, or if the activity of a polypeptide encoded by a nucleic acid molecule comprising the nucleic acid SEQ ID NO. 6040, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 6040 or polypeptide SEQ ID NO. 6041, respectively, or a homolog or a fragment thereof, is increased or generated, or if the activity beta-hydroxylase is increased or generated in a non-human organism, like a microorganism or a plant cell, plant or part thereof, especially with non-targeted localization, whereby the respective line disclose in column 7 the fine chemical triacylglycerol. For example, an increase of the triacylglycerol of at least 1 percent, particularly in a range of 31 to 40-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a oxidoreductase, or if the activity of the polypeptide AvinDRAFT_{—}3253, preferably represented by SEQ ID NO. 29501, or a homolog or fragment thereof, or if the activity of a polypeptide encoded by a nucleic acid molecule comprising the nucleic acid SEQ ID NO. 29500, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 29500 or polypeptide SEQ ID NO. 29501, respectively, or a homolog or a fragment thereof, is increased or generated, or if the activity oxidoreductase is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 38 to 80-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a ThiF family protein, or if the activity of the polypeptide AvinDRAFT_{—}3577, preferably represented by SEQ ID NO. 111156, or a homolog or fragment thereof, or if the activity of a polypeptide encoded by a nucleic acid molecule comprising the nucleic acid SEQ ID NO. 111155, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 111155 or polypeptide SEQ ID NO. 111156, respectively, or a homolog or a fragment thereof, is increased or generated, or if the activity ThiF family protein is increased or generated in a non-human organism, like a microorganism or a plant cell, plant or part thereof, especially with non-targeted localization, whereby the respective line disclose in column 7 the fine chemical glycerol phosphate (lipid fraction). For example, an increase of the glycerol phosphate (lipid fraction) of at least 1 percent, particularly in a range of 17 to 56-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a recombinase A, or if the activity of the polypeptide AvinDRAFT_{—}3629, preferably represented by SEQ ID NO. 114232, or a homolog or fragment thereof, or if the activity of a polypeptide encoded by a nucleic acid molecule comprising the nucleic acid SEQ ID NO. 114231, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 114231 or polypeptide SEQ ID NO. 114232, respectively, or a homolog or a fragment thereof, is increased or generated, or if the activity recombinase A is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 34 to 60-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a NADH-quinone oxidoreductase subunit, or if the activity of the polypeptide AvinDRAFT_{—}4606, preferably represented by SEQ ID NO. 73720, or a homolog or fragment thereof, or if the activity of a polypeptide encoded by a nucleic acid molecule comprising the nucleic acid SEQ ID NO. 73719, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 73719 or polypeptide SEQ ID NO. 73720, respectively, or a homolog or a fragment thereof, is increased or generated, or if the activity NADH-quinone oxidoreductase subunit is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 39 to 53-percent is conferred as compared to a corresponding non-transformed wild type non-human organism.

Accordingly, in one embodiment, an increase of triacylglycerol in a non-human organism, as compared to a corresponding non-transformed wild type non-human organism, is conferred in the process of the invention, if the activity of a polypeptide showing the activity of a hydrolase, or if the activity of the polypeptide AvinDRAFT_{—}5103, preferably represented by SEQ ID NO. 6511, or a homolog or fragment thereof, or if the activity of a polypeptide encoded by a nucleic acid molecule comprising the nucleic acid SEQ ID NO. 6510, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 6510 or polypeptide SEQ ID NO. 6511, respectively, or a homolog or a fragment thereof, is increased or generated, or if the activity hydrolase is increased or generated in a non-human organism, like a microorganism or a plant cell, plant or part thereof, especially with non-targeted localization, whereby the respective line disclose in column 7 the fine chemical triacylglycerol. For example, an increase of the triacylglycerol of at least 1 percent, particularly in a range of 57 to 69-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a HesB/YadR/YfhF family protein, or if the activity of the polypeptide AvinDRAFT_{—}5467, preferably represented by SEQ ID NO. 32649, or a homolog or fragment thereof, or if the activity of a polypeptide encoded by a nucleic acid molecule comprising the nucleic acid SEQ ID NO. 32648, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 32648 or polypeptide SEQ ID NO. 32649, respectively, or a homolog or a fragment thereof, is increased or generated, or if the activity HesB/YadR/YfhF family protein is increased or generated in a non-human organism, like a microorganism or a plant cell, plant or part thereof, especially with non-targeted localization, whereby the respective line disclose in column 7 the fine chemical glycerol phosphate (lipid fraction). For example, an increase of the glycerol phosphate (lipid fraction) of at least 1 percent, particularly in a range of 19 to 65-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a pyruvate kinase, or if the activity of the polypeptide AY623894, preferably represented by SEQ ID NO. 103434, or a homolog or fragment thereof, or if the activity of a polypeptide encoded by a nucleic acid molecule comprising the nucleic acid SEQ ID NO. 103433, preferably the coding region thereof, or a homolog or fragment thereof, e.g. derived from Zea mays, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 103433 or polypeptide SEQ ID NO. 103434, respectively, or a homolog or a fragment thereof, is increased or generated, or if the activity pyruvate kinase is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 37 to 93-percent is conferred as compared to a corresponding non-transformed wild type non-human organism.

Accordingly, in one embodiment, an increase of triacylglycerol in a non-human organism, as compared to a corresponding non-transformed wild type non-human organism, is conferred in the process of the invention, if the activity of a polypeptide showing the activity of a threonine synthase, or if the activity of the polypeptide B0004, preferably represented by SEQ ID NO. 34890, or a homolog or fragment thereof, or if the activity of a polypeptide encoded by a nucleic acid molecule comprising the nucleic acid SEQ ID NO. 34889, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 34889 or polypeptide SEQ ID NO. 34890, respectively, or a homolog or a fragment thereof, is increased or generated, or if the activity threonine synthase is increased or generated in a non-human organism, like a microorganism or a plant cell, plant or part thereof, especially with plastidic localization, whereby the respective line disclose in column 7 the fine chemical triacylglycerol. For example, an increase of the triacylglycerol of at least 1 percent, particularly in a range of 56 to 127-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a b0050-protein, or if the activity of the polypeptide B0050, preferably represented by SEQ ID NO. 103959, or a homolog or fragment thereof, or if the activity of a polypeptide encoded by a nucleic acid molecule comprising the nucleic acid SEQ ID NO. 103958, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 103958 or polypeptide SEQ ID NO. 103959, respectively, or a homolog or a fragment thereof, is increased or generated, or if the activity b0050-protein is increased or generated in a non-human organism, like a microorganism or a plant cell, plant or part thereof, especially with non-targeted localization, whereby the respective line disclose in column 7 the fine chemical glycerol phosphate (lipid fraction). For example, an increase of the glycerol phosphate (lipid fraction) of at least 1 percent, particularly in a range of 18 to 39-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a serine protease, or if the activity of the polypeptide B0161, preferably represented by SEQ ID NO. 7082, or a homolog or fragment thereof, or if the activity of a polypeptide encoded by a nucleic acid molecule comprising the nucleic acid SEQ ID NO. 7081, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 7081 or polypeptide SEQ ID NO. 7082, respectively, or a homolog or a fragment thereof, is increased or generated, or if the activity serine protease is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 38 to 78-percent is conferred as compared to a corresponding non-transformed wild type non-human organism.

Accordingly, in one embodiment, an increase of triacylglycerol in a non-human organism, as compared to a corresponding non-transformed wild type non-human organism, is conferred in the process of the invention, if the activity of a polypeptide showing the activity of a serine protease, or if the activity of the polypeptide B0161, preferably represented by SEQ ID NO. 7082, or a homolog or fragment thereof, or if the activity of a polypeptide encoded by a nucleic acid molecule comprising the nucleic acid SEQ ID NO. 7081, preferably the coding region thereof, or a homolog or fragment thereof, e.g. derived from Escherichia coli, is increased or generated.

For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 7081 or polypeptide SEQ ID NO. 7082, respectively, or a homolog or a fragment thereof, is increased or generated, or if the activity serine protease is increased or generated in a non-human organism, like a microorganism or a plant cell, plant or part thereof, especially with non-targeted localization, whereby the respective line disclose in column 7 the fine chemical triacylglycerol. For example, an increase of the triacylglycerol of at least 1 percent, particularly in a range of 47 to 112-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a ATP-binding component of a transport system, or if the activity of the polypeptide B0449, preferably represented by SEQ ID NO. 7334, or a homolog or fragment thereof, or if the activity of a polypeptide encoded by a nucleic acid molecule comprising the nucleic acid SEQ ID NO. 7333, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 7333 or polypeptide SEQ ID NO. 7334, respectively, or a homolog or a fragment thereof, is increased or generated, or if the activity ATP-binding component of a transport system is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 48 to 187-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a b0518-protein, or if the activity of the polypeptide B0518, preferably represented by SEQ ID NO. 35937, or a homolog or fragment thereof, or if the activity of a polypeptide encoded by a nucleic acid molecule comprising the nucleic acid SEQ ID NO. 35936, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 35936 or polypeptide SEQ ID NO. 35937, respectively, or a homolog or a fragment thereof, is increased or generated, or if the activity b0518-protein is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 25 to 54-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a phosphoglyceromutase, or if the activity of the polypeptide B0755, preferably represented by SEQ ID NO. 152577, or a homolog or fragment thereof, or if the activity of a polypeptide encoded by a nucleic acid molecule comprising the nucleic acid SEQ ID NO. 152576, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 152576 or polypeptide SEQ ID NO. 152577, respectively, or a homolog or a fragment thereof, is increased or generated, or if the activity phosphoglyceromutase is increased or generated in a non-human organism, like a microorganism or a plant cell, plant or part thereof, especially with plastidic localization, whereby the respective line disclose in column 7 the fine chemical bis-glyceryl phosphate (polar fraction). For example, an increase of the bis-glyceryl phosphate (polar fraction) of at least 1 percent, particularly in a range of 51 to 83-percent is conferred as compared to a corresponding non-transformed wild type non-human organism.

Accordingly, in one embodiment, an increase of glycerol (polar fraction) in a non-human organism, as compared to a corresponding non-transformed wild type non-human organism, is conferred in the process of the invention, if the activity of a polypeptide showing the activity of a putrescine transport system permease protein, or if the activity of the polypeptide B0856, preferably represented by SEQ ID NO. 131360, or a homolog or fragment thereof, or if the activity of a polypeptide encoded by a nucleic acid molecule comprising the nucleic acid SEQ ID NO. 131359, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 131359 or polypeptide SEQ ID NO. 131360, respectively, or a homolog or a fragment thereof, is increased or generated, or if the activity putrescine transport system permease protein is increased or generated in a non-human organism, like a microorganism or a plant cell, plant or part thereof, especially with plastidic localization, whereby the respective line disclose in column 7 the fine chemical glycerol (polar fraction). For example, an increase of the glycerol (polar fraction) of at least 1 percent, particularly in a range of 39 to 57-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a methylglyoxal synthase, or if the activity of the polypeptide B0963, preferably represented by SEQ ID NO. 36671, or a homolog or fragment thereof, or if the activity of a polypeptide encoded by a nucleic acid molecule comprising the nucleic acid SEQ ID NO. 36670, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 36670 or polypeptide SEQ ID NO. 36671, respectively, or a homolog or a fragment thereof, is increased or generated, or if the activity methylglyoxal synthase is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 16 to 23-percent is conferred as compared to a corresponding non-transformed wild type non-human organism.

Accordingly, in one embodiment, an increase of glycerol (polar fraction) in a non-human organism, as compared to a corresponding non-transformed wild type non-human organism, is conferred in the process of the invention, if the activity of a polypeptide showing the activity of a methylglyoxal synthase, or if the activity of the polypeptide B0963, preferably represented by SEQ ID NO. 36671, or a homolog or fragment thereof, or if the activity of a polypeptide encoded by a nucleic acid molecule comprising the nucleic acid SEQ ID NO. 36670, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 36670 or polypeptide SEQ ID NO. 36671, respectively, or a homolog or a fragment thereof, is increased or generated, or if the activity methylglyoxal synthase is increased or generated in a non-human organism, like a microorganism or a plant cell, plant or part thereof, especially with non-targeted localization, whereby the respective line disclose in column 7 the fine chemical glycerol (polar fraction). For example, an increase of the glycerol (polar fraction) of at least 1 percent, particularly in a range of 46 to 105-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a b0970-protein, or if the activity of the polypeptide B0970, preferably represented by SEQ ID NO. 144480, or a homolog or fragment thereof, or if the activity of a polypeptide encoded by a nucleic acid molecule comprising the nucleic acid SEQ ID NO. 144479, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 144479 or polypeptide SEQ ID NO. 144480, respectively, or a homolog or a fragment thereof, is increased or generated, or if the activity b0970-protein is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 60 to 205-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a b1003-protein, or if the activity of the polypeptide B1003, preferably represented by SEQ ID NO. 7942, or a homolog or fragment thereof, or if the activity of a polypeptide encoded by a nucleic acid molecule comprising the nucleic acid SEQ ID NO. 7941, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 7941 or polypeptide SEQ ID NO. 7942, respectively, or a homolog or a fragment thereof, is increased or generated, or if the activity b1003-protein is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 52 to 148-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a 3-oxoacyl-[acyl-carrier-protein] synthase, or if the activity of the polypeptide B1095, preferably represented by SEQ ID NO. 114553, or a homolog or fragment thereof, or if the activity of a polypeptide encoded by a nucleic acid molecule comprising the nucleic acid SEQ ID NO. 114552, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 114552 or polypeptide SEQ ID NO. 114553, respectively, or a homolog or a fragment thereof, is increased or generated, or if the activity 3-oxoacyl-[acyl-carrier-protein] synthase is increased or generated in a non-human organism, like a microorganism or a plant cell, plant or part thereof, especially with plastidic localization, whereby the respective line disclose in column 7 the fine chemical bis-glyceryl phosphate (polar fraction). For example, an increase of the bis-glyceryl phosphate (polar fraction) of at least 1 percent, particularly in a range of 53 to 164-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a sodium/proton antiporter, or if the activity of the polypeptide B1186, preferably represented by SEQ ID NO. 37401, or a homolog or fragment thereof, or if the activity of a polypeptide encoded by a nucleic acid molecule comprising the nucleic acid SEQ ID NO. 37400, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 37400 or polypeptide SEQ ID NO. 37401, respectively, or a homolog or a fragment thereof, is increased or generated, or if the activity sodium/proton antiporter is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 17 to 80-percent is conferred as compared to a corresponding non-transformed wild type non-human organism.

Accordingly, in one embodiment, an increase of triacylglycerol in a non-human organism, as compared to a corresponding non-transformed wild type non-human organism, is conferred in the process of the invention, if the activity of a polypeptide showing the activity of a b1234-protein, or if the activity of the polypeptide B1234, preferably represented by SEQ ID NO. 112112, or a homolog or fragment thereof, or if the activity of a polypeptide encoded by a nucleic acid molecule comprising the nucleic acid SEQ ID NO. 112111, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 112111 or polypeptide SEQ ID NO. 112112, respectively, or a homolog or a fragment thereof, is increased or generated, or if the activity b1234-protein is increased or generated in a non-human organism, like a microorganism or a plant cell, plant or part thereof, especially with non-targeted localization, whereby the respective line disclose in column 7 the fine chemical triacylglycerol. For example, an increase of the triacylglycerol of at least 1 percent, particularly in a range of 41 to 80-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a cardiolipin synthase, or if the activity of the polypeptide B1249, preferably represented by SEQ ID NO. 98779, or a homolog or fragment thereof, or if the activity of a polypeptide encoded by a nucleic acid molecule comprising the nucleic acid SEQ ID NO. 98778, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 98778 or polypeptide SEQ ID NO. 98779, respectively, or a homolog or a fragment thereof, is increased or generated, or if the activity cardiolipin synthase is increased or generated in a non-human organism, like a microorganism or a plant cell, plant or part thereof, especially with plastidic localization, whereby the respective line disclose in column 7 the fine chemical glycerol (lipid fraction). For example, an increase of the glycerol (lipid fraction) of at least 1 percent, particularly in a range of 10 to 22-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a b1330-protein, or if the activity of the polypeptide B1330, preferably represented by SEQ ID NO. 38227, or a homolog or fragment thereof, or if the activity of a polypeptide encoded by a nucleic acid molecule comprising the nucleic acid SEQ ID NO. 38226, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 38226 or polypeptide SEQ ID NO. 38227, respectively, or a homolog or a fragment thereof, is increased or generated, or if the activity b1330-protein is increased or generated in a non-human organism, like a microorganism or a plant cell, plant or part thereof, especially with non-targeted localization, whereby the respective line disclose in column 7 the fine chemical glycerol phosphate (lipid fraction). For example, an increase of the glycerol phosphate (lipid fraction) of at least 1 percent, particularly in a range of 25 to 55-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a ATP-dependent RNA helicase, or if the activity of the polypeptide B1343, preferably represented by SEQ ID NO. 76891, or a homolog or fragment thereof, or if the activity of a polypeptide encoded by a nucleic acid molecule comprising the nucleic acid SEQ ID NO. 76890, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 76890 or polypeptide SEQ ID NO. 76891, respectively, or a homolog or a fragment thereof, is increased or generated, or if the activity ATP-dependent RNA helicase is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 38 to 51-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a phenylacetic acid degradation protein, or if the activity of the polypeptide B1391, preferably represented by SEQ ID NO. 131490, or a homolog or fragment thereof, or if the activity of a polypeptide encoded by a nucleic acid molecule comprising the nucleic acid SEQ ID NO. 131489, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 131489 or polypeptide SEQ ID NO. 131490, respectively, or a homolog or a fragment thereof, is increased or generated, or if the activity phenylacetic acid degradation protein is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 44 to 74-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a lipoprotein precursor, or if the activity of the polypeptide B1431, preferably represented by SEQ ID NO. 38267, or a homolog or fragment thereof, or if the activity of a polypeptide encoded by a nucleic acid molecule comprising the nucleic acid SEQ ID NO. 38266, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 38266 or polypeptide SEQ ID NO. 38267, respectively, or a homolog or a fragment thereof, is increased or generated, or if the activity lipoprotein precursor is increased or generated in a non-human organism, like a microorganism or a plant cell, plant or part thereof, especially with plastidic localization, whereby the respective line disclose in column 7 the fine chemical bis-glyceryl phosphate (polar fraction). For example, an increase of the bis-glyceryl phosphate (polar fraction) of at least 1 percent, particularly in a range of 45 to 94-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a b1522-protein, or if the activity of the polypeptide B1522, preferably represented by SEQ ID NO. 7948, or a homolog or fragment thereof, or if the activity of a polypeptide encoded by a nucleic acid molecule comprising the nucleic acid SEQ ID NO. 7947, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 7947 or polypeptide SEQ ID NO. 7948, respectively, or a homolog or a fragment thereof, is increased or generated, or if the activity b1522-protein is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 64 to 135-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a lysozyme, or if the activity of the polypeptide B1554, preferably represented by SEQ ID NO. 152992, or a homolog or fragment thereof, or if the activity of a polypeptide encoded by a nucleic acid molecule comprising the nucleic acid SEQ ID NO. 152991, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 152991 or polypeptide SEQ ID NO. 152992, respectively, or a homolog or a fragment thereof, is increased or generated, or if the activity lysozyme is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 39 to 74-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a b1556-protein, or if the activity of the polypeptide B1556, preferably represented by SEQ ID NO. 145143, or a homolog or fragment thereof, or if the activity of a polypeptide encoded by a nucleic acid molecule comprising the nucleic acid SEQ ID NO. 145142, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 145142 or polypeptide SEQ ID NO. 145143, respectively, or a homolog or a fragment thereof, is increased or generated, or if the activity b1556-protein is increased or generated in a non-human organism, like a microorganism or a plant cell, plant or part thereof, especially with plastidic localization, whereby the respective line disclose in column 7 the fine chemical bis-glyceryl phosphate (polar fraction). For example, an increase of the bis-glyceryl phosphate (polar fraction) of at least 1 percent, particularly in a range of 44 to 119-percent is conferred as compared to a corresponding non-transformed wild type non-human organism.

Accordingly, in one embodiment, an increase of triacylglycerol in a non-human organism, as compared to a corresponding non-transformed wild type non-human organism, is conferred in the process of the invention, if the activity of a polypeptide showing the activity of a b1583-protein, or if the activity of the polypeptide B1583, preferably represented by SEQ ID NO. 78139, or a homolog or fragment thereof, or if the activity of a polypeptide encoded by a nucleic acid molecule comprising the nucleic acid SEQ ID NO. 78138, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 78138 or polypeptide SEQ ID NO. 78139, respectively, or a homolog or a fragment thereof, is increased or generated, or if the activity b1583-protein is increased or generated in a non-human organism, like a microorganism or a plant cell, plant or part thereof, especially with non-targeted localization, whereby the respective line disclose in column 7 the fine chemical triacylglycerol. For example, an increase of the triacylglycerol of at least 1 percent, particularly in a range of 30 to 32-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a Fe—S subunit of oxidoreductase, or if the activity of the polypeptide B1589, preferably represented by SEQ ID NO. 78155, or a homolog or fragment thereof, or if the activity of a polypeptide encoded by a nucleic acid molecule comprising the nucleic acid SEQ ID NO. 78154, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 78154 or polypeptide SEQ ID NO. 78155, respectively, or a homolog or a fragment thereof, is increased or generated, or if the activity Fe—S subunit of oxidoreductase is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 41 to 94-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a acid shock protein, or if the activity of the polypeptide B1597, preferably represented by SEQ ID NO. 38301, or a homolog or fragment thereof, or if the activity of a polypeptide encoded by a nucleic acid molecule comprising the nucleic acid SEQ ID NO. 38300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 38300 or polypeptide SEQ ID NO. 38301, respectively, or a homolog or a fragment thereof, is increased or generated, or if the activity acid shock protein is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 21 to 123-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a b1670-protein, or if the activity of the polypeptide B1670, preferably represented by SEQ ID NO. 78754, or a homolog or fragment thereof, or if the activity of a polypeptide encoded by a nucleic acid molecule comprising the nucleic acid SEQ ID NO. 78753, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 78753 or polypeptide SEQ ID NO. 78754, respectively, or a homolog or a fragment thereof, is increased or generated, or if the activity b1670-protein is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 53 to 94-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a b1672-protein, or if the activity of the polypeptide B1672, preferably represented by SEQ ID NO. 78772, or a homolog or fragment thereof, or if the activity of a polypeptide encoded by a nucleic acid molecule comprising the nucleic acid SEQ ID NO. 78771, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 78771 or polypeptide SEQ ID NO. 78772, respectively, or a homolog or a fragment thereof, is increased or generated, or if the activity b1672-protein is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 39 to 72-percent is conferred as compared to a corresponding non-transformed wild type non-human organism.

Accordingly, in one embodiment, an increase of triacylglycerol in a non-human organism, as compared to a corresponding non-transformed wild type non-human organism, is conferred in the process of the invention, if the activity of a polypeptide showing the activity of a b1703-protein, or if the activity of the polypeptide B1703, preferably represented by SEQ ID NO. 124908, or a homolog or fragment thereof, or if the activity of a polypeptide encoded by a nucleic acid molecule comprising the nucleic acid SEQ ID NO. 124907, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 124907 or polypeptide SEQ ID NO. 124908, respectively, or a homolog or a fragment thereof, is increased or generated, or if the activity b1703-protein is increased or generated in a non-human organism, like a microorganism or a plant cell, plant or part thereof, especially with non-targeted localization, whereby the respective line disclose in column 7 the fine chemical triacylglycerol. For example, an increase of the triacylglycerol of at least 1 percent, particularly in a range of 35 to 49-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a phosphotransferase system component, or if the activity of the polypeptide B1736, preferably represented by SEQ ID NO. 153073, or a homolog or fragment thereof, or if the activity of a polypeptide encoded by a nucleic acid molecule comprising the nucleic acid SEQ ID NO. 153072, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 153072 or polypeptide SEQ ID NO. 153073, respectively, or a homolog or a fragment thereof, is increased or generated, or if the activity phosphotransferase system component is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 38 to 85-percent is conferred as compared to a corresponding non-transformed wild type non-human organism.

Accordingly, in one embodiment, an increase of triacylglycerol in a non-human organism, as compared to a corresponding non-transformed wild type non-human organism, is conferred in the process of the invention, if the activity of a polypeptide showing the activity of a B1795-protein, or if the activity of the polypeptide B1795, preferably represented by SEQ ID NO. 131540, or a homolog or fragment thereof, or if the activity of a polypeptide encoded by a nucleic acid molecule comprising the nucleic acid SEQ ID NO. 131539, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 131539 or polypeptide SEQ ID NO. 131540, respectively, or a homolog or a fragment thereof, is increased or generated, or if the activity B1795-protein is increased or generated in a non-human organism, like a microorganism or a plant cell, plant or part thereof, especially with non-targeted localization, whereby the respective line disclose in column 7 the fine chemical triacylglycerol. For example, an increase of the triacylglycerol of at least 1 percent, particularly in a range of 40 to 82-percent is conferred as compared to a corresponding non-transformed wild type non-human organism.

Accordingly, in one embodiment, an increase of glycerol-3-phosphate (polar fraction) in a non-human organism, as compared to a corresponding non-transformed wild type non-human organism, is conferred in the process of the invention, if the activity of a polypeptide showing the activity of a L-serine dehydratase, or if the activity of the polypeptide B1814, preferably represented by SEQ ID NO. 8034, or a homolog or fragment thereof, or if the activity of a polypeptide encoded by a nucleic acid molecule comprising the nucleic acid SEQ ID NO. 8033, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 8033 or polypeptide SEQ ID NO. 8034, respectively, or a homolog or a fragment thereof, is increased or generated, or if the activity L-serine dehydratase is increased or generated in a non-human organism, like a microorganism or a plant cell, plant or part thereof, especially with plastidic localization, whereby the respective line disclose in column 7 the fine chemical glycerol-3-phosphate (polar fraction). For example, an increase of the glycerol-3-phosphate (polar fraction) of at least 1-percent, particularly in a range of 54 to 199-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a b1837-protein, or if the activity of the polypeptide B1837, preferably represented by SEQ ID NO. 78954, or a homolog or fragment thereof, or if the activity of a polypeptide encoded by a nucleic acid molecule comprising the nucleic acid SEQ ID NO. 78953, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 78953 or polypeptide SEQ ID NO. 78954, respectively, or a homolog or a fragment thereof, is increased or generated, or if the activity b1837-protein is increased or generated in a non-human organism, like a microorganism or a plant cell, plant or part thereof, especially with non-targeted localization, whereby the respective line disclose in column 7 the fine chemical glycerol phosphate (lipid fraction). For example, an increase of the glycerol phosphate (lipid fraction) of at least 1 percent, particularly in a range of 20 to 38-percent is conferred as compared to a corresponding non-transformed wild type non-human organism.

Accordingly, in one embodiment, an increase of triacylglycerol in a non-human organism, as compared to a corresponding non-transformed wild type non-human organism, is conferred in the process of the invention, if the activity of a polypeptide showing the activity of a trehalosephosphatase, or if the activity of the polypeptide B1897, preferably represented by SEQ ID NO. 79057, or a homolog or fragment thereof, or if the activity of a polypeptide encoded by a nucleic acid molecule comprising the nucleic acid SEQ ID NO. 79056, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 79056 or polypeptide SEQ ID NO. 79057, respectively, or a homolog or a fragment thereof, is increased or generated, or if the activity trehalose-phosphatase is increased or generated in a non-human organism, like a microorganism or a plant cell, plant or part thereof, especially with non-targeted localization, whereby the respective line disclose in column 7 the fine chemical triacylglycerol. For example, an increase of the triacylglycerol of at least 1 percent, particularly in a range of 41 to 60-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a imidazole glycerol phosphate synthase subunit, or if the activity of the polypeptide B2023, preferably represented by SEQ ID NO. 99372, or a homolog or fragment thereof, or if the activity of a polypeptide encoded by a nucleic acid molecule comprising the nucleic acid SEQ ID NO. 99371, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 99371 or polypeptide SEQ ID NO. 99372, respectively, or a homolog or a fragment thereof, is increased or generated, or if the activity imidazole glycerol phosphate synthase subunit is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 13 to 19-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a b2032-protein, or if the activity of the polypeptide B2032, preferably represented by SEQ ID NO. 8921, or a homolog or fragment thereof, or if the activity of a polypeptide encoded by a nucleic acid molecule comprising the nucleic acid SEQ ID NO. 8920, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 8920 or polypeptide SEQ ID NO. 8921, respectively, or a homolog or a fragment thereof, is increased or generated, or if the activity b2032-protein is increased or generated in a non-human organism, like a microorganism or a plant cell, plant or part thereof, especially with non-targeted localization, whereby the respective line disclose in column 7 the fine chemical glycerol phosphate (lipid fraction). For example, an increase of the glycerol phosphate (lipid fraction) of at least 1 percent, particularly in a range of 22 to 67-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a b2099-protein, or if the activity of the polypeptide B2099, preferably represented by SEQ ID NO. 115596, or a homolog or fragment thereof, or if the activity of a polypeptide encoded by a nucleic acid molecule comprising the nucleic acid SEQ ID NO. 115595, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 115595 or polypeptide SEQ ID NO. 115596, respectively, or a homolog or a fragment thereof, is increased or generated, or if the activity b2099-protein is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 16 to 25-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a b2139-protein, or if the activity of the polypeptide B2139, preferably represented by SEQ ID NO. 125086, or a homolog or fragment thereof, or if the activity of a polypeptide encoded by a nucleic acid molecule comprising the nucleic acid SEQ ID NO. 125085, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 125085 or polypeptide SEQ ID NO. 125086, respectively, or a homolog or a fragment thereof, is increased or generated, or if the activity b2139-protein is increased or generated in a non-human organism, like a microorganism or a plant cell, plant or part thereof, especially with plastidic localization, whereby the respective line disclose in column 7 the fine chemical bis-glyceryl phosphate (polar fraction). For example, an increase of the bis-glyceryl phosphate (polar fraction) of at least 1 percent, particularly in a range of 42 to 43-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a ABC transporter permease protein, or if the activity of the polypeptide B2178, preferably represented by SEQ ID NO. 39041, or a homolog or fragment thereof, or if the activity of a polypeptide encoded by a nucleic acid molecule comprising the nucleic acid SEQ ID NO. 39040, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 39040 or polypeptide SEQ ID NO. 39041, respectively, or a homolog or a fragment thereof, is increased or generated, or if the activity ABC transporter permease protein is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 17 to 44-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a ion-transport protein, or if the activity of the polypeptide B2389, preferably represented by SEQ ID NO. 146239, or a homolog or fragment thereof, or if the activity of a polypeptide encoded by a nucleic acid molecule comprising the nucleic acid SEQ ID NO. 146238, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 146238 or polypeptide SEQ ID NO. 146239, respectively, or a homolog or a fragment thereof, is increased or generated, or if the activity ion-transport protein is increased or generated in a non-human organism, like a microorganism or a plant cell, plant or part thereof, especially with non-targeted localization, whereby the respective line disclose in column 7 the fine chemical glycerol phosphate (lipid fraction). For example, an increase of the glycerol phosphate (lipid fraction) of at least 1 percent, particularly in a range of 21 to 34-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a b2399-protein, or if the activity of the polypeptide B2399, preferably represented by SEQ ID NO. 39238, or a homolog or fragment thereof, or if the activity of a polypeptide encoded by a nucleic acid molecule comprising the nucleic acid SEQ ID NO. 39237, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 39237 or polypeptide SEQ ID NO. 39238, respectively, or a homolog or a fragment thereof, is increased or generated, or if the activity b2399-protein is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 53 to 471-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a b2399-protein, or if the activity of the polypeptide B2399, preferably represented by SEQ ID NO. 39238, or a homolog or fragment thereof, or if the activity of a polypeptide encoded by a nucleic acid molecule comprising the nucleic acid SEQ ID NO. 39237, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 39237 or polypeptide SEQ ID NO. 39238, respectively, or a homolog or a fragment thereof, is increased or generated, or if the activity b2399-protein is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 20 to 45-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a ethanolamine utilization protein, or if the activity of the polypeptide B2439, preferably represented by SEQ ID NO. 115629, or a homolog or fragment thereof, or if the activity of a polypeptide encoded by a nucleic acid molecule comprising the nucleic acid SEQ ID NO. 115628, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 115628 or polypeptide SEQ ID NO. 115629, respectively, or a homolog or a fragment thereof, is increased or generated, or if the activity ethanolamine utilization protein is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 17 to 35-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a ethanolamine utilization protein, or if the activity of the polypeptide B2439, preferably represented by SEQ ID NO. 115629, or a homolog or fragment thereof, or if the activity of a polypeptide encoded by a nucleic acid molecule comprising the nucleic acid SEQ ID NO. 115628, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 115628 or polypeptide SEQ ID NO. 115629, respectively, or a homolog or a fragment thereof, is increased or generated, or if the activity ethanolamine utilization protein is increased or generated in a non-human organism, like a microorganism or a plant cell, plant or part thereof, especially with non-targeted localization, whereby the respective line disclose in column 7 the fine chemical glycerol phosphate (lipid fraction). For example, an increase of the glycerol phosphate (lipid fraction) of at least 1 percent, particularly in a range of 21 to 31-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a ethanolamine utilization protein, or if the activity of the polypeptide B2461, preferably represented by SEQ ID NO. 40300, or a homolog or fragment thereof, or if the activity of a polypeptide encoded by a nucleic acid molecule comprising the nucleic acid SEQ ID NO. 40299, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 40299 or polypeptide SEQ ID NO. 40300, respectively, or a homolog or a fragment thereof, is increased or generated, or if the activity ethanolamine utilization protein is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 20 to 42-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a b2474-protein, or if the activity of the polypeptide B2474, preferably represented by SEQ ID NO. 40330, or a homolog or fragment thereof, or if the activity of a polypeptide encoded by a nucleic acid molecule comprising the nucleic acid SEQ ID NO. 40329, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 40329 or polypeptide SEQ ID NO. 40330, respectively, or a homolog or a fragment thereof, is increased or generated, or if the activity b2474-protein is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 38 to 83-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a b2474-protein, or if the activity of the polypeptide B2474, preferably represented by SEQ ID NO. 40330, or a homolog or fragment thereof, or if the activity of a polypeptide encoded by a nucleic acid molecule comprising the nucleic acid SEQ ID NO. 40329, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 40329 or polypeptide SEQ ID NO. 40330, respectively, or a homolog or a fragment thereof, is increased or generated, or if the activity b2474-protein is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 16 to 28-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a putative transport system permease protein, or if the activity of the polypeptide B2546, preferably represented by SEQ ID NO. 99899, or a homolog or fragment thereof, or if the activity of a polypeptide encoded by a nucleic acid molecule comprising the nucleic acid SEQ ID NO. 99898, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 99898 or polypeptide SEQ ID NO. 99899, respectively, or a homolog or a fragment thereof, is increased or generated, or if the activity putative transport system permease protein is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 22 to 24-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a putative transport system permease protein, or if the activity of the polypeptide B2546, preferably represented by SEQ ID NO. 99899, or a homolog or fragment thereof, or if the activity of a polypeptide encoded by a nucleic acid molecule comprising the nucleic acid SEQ ID NO. 99898, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 99898 or polypeptide SEQ ID NO. 99899, respectively, or a homolog or a fragment thereof, is increased or generated, or if the activity putative transport system permease protein is increased or generated in a non-human organism, like a microorganism or a plant cell, plant or part thereof, especially with non-targeted localization, whereby the respective line disclose in column 7 the fine chemical glycerol phosphate (lipid fraction). For example, an increase of the glycerol phosphate (lipid fraction) of at least 1 percent, particularly in a range of 18 to 52-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a b2613-protein, or if the activity of the polypeptide B2613, preferably represented by SEQ ID NO. 40666, or a homolog or fragment thereof, or if the activity of a polypeptide encoded by a nucleic acid molecule comprising the nucleic acid SEQ ID NO. 40665, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 40665 or polypeptide SEQ ID NO. 40666, respectively, or a homolog or a fragment thereof, is increased or generated, or if the activity b2613-protein is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 42 to 82-percent is conferred as compared to a corresponding non-transformed wild type non-human organism.

Accordingly, in one embodiment, an increase of glycerol (polar fraction) in a non-human organism, as compared to a corresponding non-transformed wild type non-human organism, is conferred in the process of the invention, if the activity of a polypeptide showing the activity of a transcriptional regulator, or if the activity of the polypeptide B2634, preferably represented by SEQ ID NO. 40727, or a homolog or fragment thereof, or if the activity of a polypeptide encoded by a nucleic acid molecule comprising the nucleic acid SEQ ID NO. 40726, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 40726 or polypeptide SEQ ID NO. 40727, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line disclose in column 7 the fine chemical glycerol (polar fraction). For example, an increase of the glycerol (polar fraction) of at least 1 percent, particularly in a range of 58 to 125-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a b2673-protein, or if the activity of the polypeptide B2673, preferably represented by SEQ ID NO. 9245, or a homolog or fragment thereof, or if the activity of a polypeptide encoded by a nucleic acid molecule comprising the nucleic acid SEQ ID NO. 9244, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 9244 or polypeptide SEQ ID NO. 9245, respectively, or a homolog or a fragment thereof, is increased or generated, or if the activity b2673-protein is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 17 to 20-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a b2673-protein, or if the activity of the polypeptide B2673, preferably represented by SEQ ID NO. 9245, or a homolog or fragment thereof, or if the activity of a polypeptide encoded by a nucleic acid molecule comprising the nucleic acid SEQ ID NO. 9244, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 9244 or polypeptide SEQ ID NO. 9245, respectively, or a homolog or a fragment thereof, is increased or generated, or if the activity b2673-protein is increased or generated in a non-human organism, like a microorganism or a plant cell, plant or part thereof, especially with non-targeted localization, whereby the respective line disclose in column 7 the fine chemical glycerol phosphate (lipid fraction). For example, an increase of the glycerol phosphate (lipid fraction) of at least 1 percent, particularly in a range of 19 to 47-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a murein transglycosylase, or if the activity of the polypeptide B2701, preferably represented by SEQ ID NO. 80757, or a homolog or fragment thereof, or if the activity of a polypeptide encoded by a nucleic acid molecule comprising the nucleic acid SEQ ID NO. 80756, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 80756 or polypeptide SEQ ID NO. 80757, respectively, or a homolog or a fragment thereof, is increased or generated, or if the activity murein transglycosylase is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 55 to 98-percent is conferred as compared to a corresponding non-transformed wild type non-human organism.

Accordingly, in one embodiment, an increase of triacylglycerol in a non-human organism, as compared to a corresponding non-transformed wild type non-human organism, is conferred in the process of the invention, if the activity of a polypeptide showing the activity of a transcriptional regulator, or if the activity of the polypeptide B2714, preferably represented by SEQ ID NO. 40742, or a homolog or fragment thereof, or if the activity of a polypeptide encoded by a nucleic acid molecule comprising the nucleic acid SEQ ID NO. 40741, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 40741 or polypeptide SEQ ID NO. 40742, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line disclose in column 7 the fine chemical triacylglycerol. For example, an increase of the triacylglycerol of at least 1 percent, particularly in a range of 38 to 56-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a 2-C-methyl-D-erythritol 4-phosphate cytidylyltransferase, or if the activity of the polypeptide B2747, preferably represented by SEQ ID NO. 81076, or a homolog or fragment thereof, or if the activity of a polypeptide encoded by a nucleic acid molecule comprising the nucleic acid SEQ ID NO. 81075, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 81075 or polypeptide SEQ ID NO. 81076, respectively, or a homolog or a fragment thereof, is increased or generated, or if the activity 2-C-methyl-D-erythritol 4-phosphate cytidylyltransferase is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 18 to 58-percent is conferred as compared to a corresponding non-transformed wild type non-human organism.

Accordingly, in one embodiment, an increase of triacylglycerol in a non-human organism, as compared to a corresponding non-transformed wild type non-human organism, is conferred in the process of the invention, if the activity of a polypeptide showing the activity of a amino-acid acetyltransferase, or if the activity of the polypeptide B2818, preferably represented by SEQ ID NO. 81577, or a homolog or fragment thereof, or if the activity of a polypeptide encoded by a nucleic acid molecule comprising the nucleic acid SEQ ID NO. 81576, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 81576 or polypeptide SEQ ID NO. 81577, respectively, or a homolog or a fragment thereof, is increased or generated, or if the activity amino-acid acetyltransferase is increased or generated in a non-human organism, like a microorganism or a plant cell, plant or part thereof, especially with non-targeted localization, whereby the respective line disclose in column 7 the fine chemical triacylglycerol. For example, an increase of the triacylglycerol of at least 1 percent, particularly in a range of 30 to 47-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a b3083-protein, or if the activity of the polypeptide B3083, preferably represented by SEQ ID NO. 82425, or a homolog or fragment thereof, or if the activity of a polypeptide encoded by a nucleic acid molecule comprising the nucleic acid SEQ ID NO. 82424, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 82424 or polypeptide SEQ ID NO. 82425, respectively, or a homolog or a fragment thereof, is increased or generated, or if the activity b3083-protein is increased or generated in a non-human organism, like a microorganism or a plant cell, plant or part thereof, especially with non-targeted localization, whereby the respective line disclose in column 7 the fine chemical bis-glyceryl phosphate (polar fraction).

For example, an increase of the bis-glyceryl phosphate (polar fraction) of at least 1 percent, particularly in a range of 43 to 60-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a threonine/serine transporter, or if the activity of the polypeptide B3116, preferably represented by SEQ ID NO. 153247, or a homolog or fragment thereof, or if the activity of a polypeptide encoded by a nucleic acid molecule comprising the nucleic acid SEQ ID NO. 153246, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 153246 or polypeptide SEQ ID NO. 153247, respectively, or a homolog or a fragment thereof, is increased or generated, or if the activity threonine/serine transporter is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 38 to 121-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a b3160-protein, or if the activity of the polypeptide B3160, preferably represented by SEQ ID NO. 153396, or a homolog or fragment thereof, or if the activity of a polypeptide encoded by a nucleic acid molecule comprising the nucleic acid SEQ ID NO. 153395, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 153395 or polypeptide SEQ ID NO. 153396, respectively, or a homolog or a fragment thereof, is increased or generated, or if the activity b3160-protein is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 47 to 100-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a argininosuccinate synthetase, or if the activity of the polypeptide B3172, preferably represented by SEQ ID NO. 153619, or a homolog or fragment thereof, or if the activity of a polypeptide encoded by a nucleic acid molecule comprising the nucleic acid SEQ ID NO. 153618, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 153618 or polypeptide SEQ ID NO. 153619, respectively, or a homolog or a fragment thereof, is increased or generated, or if the activity argininosuccinate synthetase is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 47 to 115-percent is conferred as compared to a corresponding non-transformed wild type non-human organism.

Accordingly, in one embodiment, an increase of glycerol-3-phosphate (polar fraction) in a non-human organism, as compared to a corresponding non-transformed wild type non-human organism, is conferred in the process of the invention, if the activity of a polypeptide showing the activity of a acetyl CoA carboxylase, or if the activity of the polypeptide B3256, preferably represented by SEQ ID NO. 9493, or a homolog or fragment thereof, or if the activity of a polypeptide encoded by a nucleic acid molecule comprising the nucleic acid SEQ ID NO. 9492, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 9492 or polypeptide SEQ ID NO. 9493, respectively, or a homolog or a fragment thereof, is increased or generated, or if the activity acetyl CoA carboxylase is increased or generated in a non-human organism, like a microorganism or a plant cell, plant or part thereof, especially with non-targeted localization, whereby the respective line disclose in column 7 the fine chemical glycerol-3-phosphate (polar fraction). For example, an increase of the glycerol-3-phosphate (polar fraction) of at least 1 percent, particularly in a range of 72 to 110-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a methyltransferase, or if the activity of the polypeptide B3262, preferably represented by SEQ ID NO. 42503, or a homolog or fragment thereof, or if the activity of a polypeptide encoded by a nucleic acid molecule comprising the nucleic acid SEQ ID NO. 42502, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 42502 or polypeptide SEQ ID NO. 42503, respectively, or a homolog or a fragment thereof, is increased or generated, or if the activity methyltransferase is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 59 to 124-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a methyltransferase, or if the activity of the polypeptide B3262, preferably represented by SEQ ID NO. 42503, or a homolog or fragment thereof, or if the activity of a polypeptide encoded by a nucleic acid molecule comprising the nucleic acid SEQ ID NO. 42502, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 42502 or polypeptide SEQ ID NO. 42503, respectively, or a homolog or a fragment thereof, is increased or generated, or if the activity methyltransferase is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 16 to 60-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a FKBP-type peptidyl-prolyl cis-trans isomerase (rotamase), or if the activity of the polypeptide B3347, preferably represented by SEQ ID NO. 153814, or a homolog or fragment thereof, or if the activity of a polypeptide encoded by a nucleic acid molecule comprising the nucleic acid SEQ ID NO. 153813, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 153813 or polypeptide SEQ ID NO. 153814, respectively, or a homolog or a fragment thereof, is increased or generated, or if the activity FKBP-type peptidyl-prolyl cis-trans isomerase (rotamase) is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 60 to 198-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a high-affinity branched-chain amino acid transport protein, or if the activity of the polypeptide B3457, preferably represented by SEQ ID NO. 146473, or a homolog or fragment thereof, or if the activity of a polypeptide encoded by a nucleic acid molecule comprising the nucleic acid SEQ ID NO. 146472, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 146472 or polypeptide SEQ ID NO. 146473, respectively, or a homolog or a fragment thereof, is increased or generated, or if the activity high-affinity branched-chain amino acid transport protein is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 37 to 71-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a b3777-protein, or if the activity of the polypeptide B3777, preferably represented by SEQ ID NO. 136099, or a homolog or fragment thereof, or if the activity of a polypeptide encoded by a nucleic acid molecule comprising the nucleic acid SEQ ID NO. 136098, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 136098 or polypeptide SEQ ID NO. 136099, respectively, or a homolog or a fragment thereof, is increased or generated, or if the activity b3777-protein is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 39 to 85-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a b3791-protein, or if the activity of the polypeptide B3791, preferably represented by SEQ ID NO. 83727, or a homolog or fragment thereof, or if the activity of a polypeptide encoded by a nucleic acid molecule comprising the nucleic acid SEQ ID NO. 83726, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 83726 or polypeptide SEQ ID NO. 83727, respectively, or a homolog or a fragment thereof, is increased or generated, or if the activity b3791-protein is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 40 to 93-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a enterobacterial common antigen polymerase, or if the activity of the polypeptide B3793, preferably represented by SEQ ID NO. 43801, or a homolog or fragment thereof, or if the activity of a polypeptide encoded by a nucleic acid molecule comprising the nucleic acid SEQ ID NO. 43800, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 43800 or polypeptide SEQ ID NO. 43801, respectively, or a homolog or a fragment thereof, is increased or generated, or if the activity enterobacterial common antigen polymerase is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 38 to 117-percent is conferred as compared to a corresponding non-transformed wild type non-human organism.

Accordingly, in one embodiment, an increase of triacylglycerol in a non-human organism, as compared to a corresponding non-transformed wild type non-human organism, is conferred in the process of the invention, if the activity of a polypeptide showing the activity of a b3989-protein, or if the activity of the polypeptide B3989, preferably represented by SEQ ID NO. 44373, or a homolog or fragment thereof, or if the activity of a polypeptide encoded by a nucleic acid molecule comprising the nucleic acid SEQ ID NO. 44372, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 44372 or polypeptide SEQ ID NO. 44373, respectively, or a homolog or a fragment thereof, is increased or generated, or if the activity b3989-protein is increased or generated in a non-human organism, like a microorganism or a plant cell, plant or part thereof, especially with non-targeted localization, whereby the respective line disclose in column 7 the fine chemical triacylglycerol. For example, an increase of the triacylglycerol of at least 1 percent, particularly in a range of 33 to 109-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a b4029-protein, or if the activity of the polypeptide B4029, preferably represented by SEQ ID NO. 10741, or a homolog or fragment thereof, or if the activity of a polypeptide encoded by a nucleic acid molecule comprising the nucleic acid SEQ ID NO. 10740, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 10740 or polypeptide SEQ ID NO. 10741, respectively, or a homolog or a fragment thereof, is increased or generated, or if the activity b4029-protein is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 38 to 220-percent is conferred as compared to a corresponding non-transformed wild type non-human organism.

Accordingly, in one embodiment, an increase of triacylglycerol in a non-human organism, as compared to a corresponding non-transformed wild type non-human organism, is conferred in the process of the invention, if the activity of a polypeptide showing the activity of a b4050-protein, or if the activity of the polypeptide B4050, preferably represented by SEQ ID NO. 106533, or a homolog or fragment thereof, or if the activity of a polypeptide encoded by a nucleic acid molecule comprising the nucleic acid SEQ ID NO. 106532, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 106532 or polypeptide SEQ ID NO. 106533, respectively, or a homolog or a fragment thereof, is increased or generated, or if the activity b4050-protein is increased or generated in a non-human organism, like a microorganism or a plant cell, plant or part thereof, especially with non-targeted localization, whereby the respective line disclose in column 7 the fine chemical triacylglycerol. For example, an increase of the triacylglycerol of at least 1 percent, particularly in a range of 50 to 173-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a lysyl-tRNA synthetase, or if the activity of the polypeptide B4129, preferably represented by SEQ ID NO. 44663, or a homolog or fragment thereof, or if the activity of a polypeptide encoded by a nucleic acid molecule comprising the nucleic acid SEQ ID NO. 44662, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 44662 or polypeptide SEQ ID NO. 44663, respectively, or a homolog or a fragment thereof, is increased or generated, or if the activity lysyl-tRNA synthetase is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 39 to 79-percent is conferred as compared to a corresponding non-transformed wild type non-human organism.

Accordingly, in one embodiment, an increase of glycerol-3-phosphate (polar fraction) in a non-human organism, as compared to a corresponding non-transformed wild type non-human organism, is conferred in the process of the invention, if the activity of a polypeptide showing the activity of a purine-nucleoside phosphorylase, or if the activity of the polypeptide B4384, preferably represented by SEQ ID NO. 45557, or a homolog or fragment thereof, or if the activity of a polypeptide encoded by a nucleic acid molecule comprising the nucleic acid SEQ ID NO. 45556, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 45556 or polypeptide SEQ ID NO. 45557, respectively, or a homolog or a fragment thereof, is increased or generated, or if the activity purine-nucleoside phosphorylase is increased or generated in a non-human organism, like a microorganism or a plant cell, plant or part thereof, especially with non-targeted localization, whereby the respective line disclose in column 7 the fine chemical glycerol-3-phosphate (polar fraction). For example, an increase of the glycerol-3-phosphate (polar fraction) of at least 1 percent, particularly in a range of 45 to 83-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a glutaredoxin, or if the activity of the polypeptide GM02LC12622, preferably represented by SEQ ID NO. 10812, or a homolog or fragment thereof, or if the activity of a polypeptide encoded by a nucleic acid molecule comprising the nucleic acid SEQ ID NO. 10811, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 10811 or polypeptide SEQ ID NO. 10812, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 41 to 98-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a glutaredoxin, or if the activity of the polypeptide GM02LC12622, preferably represented by SEQ ID NO. 10812, or a homolog or fragment thereof, or if the activity of a polypeptide encoded by a nucleic acid molecule comprising the nucleic acid SEQ ID NO. 10811, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 10811 or polypeptide SEQ ID NO. 10812, respectively, or a homolog or a fragment thereof, is increased or generated, or if the activity glutaredoxin is increased or generated in a non-human organism, like a microorganism or a plant cell, plant or part thereof, especially with non-targeted localization, whereby the respective line disclose in column 7 the fine chemical glycerol phosphate (lipid fraction). For example, an increase of the glycerol phosphate (lipid fraction) of at least 1 percent, particularly in a range of 18 to 34-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a GM02LC17485-protein, or if the activity of the polypeptide GM02LC17485, preferably represented by SEQ ID NO. 46406, or a homolog or fragment thereof, or if the activity of a polypeptide encoded by a nucleic acid molecule comprising the nucleic acid SEQ ID NO. 46405, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 46405 or polypeptide SEQ ID NO. 46406, respectively, or a homolog or a fragment thereof, is increased or generated, or if the activity GM02LC17485-protein is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 69 to 112-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a transcription factor, or if the activity of the polypeptide GM02LC19289, preferably represented by SEQ ID NO. 46752, or a homolog or fragment thereof, or if the activity of a polypeptide encoded by a nucleic acid molecule comprising the nucleic acid SEQ ID NO. 46751, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 46751 or polypeptide SEQ ID NO. 46752, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 25 to 59-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a transcription factor, or if the activity of the polypeptide GM02LC19289, preferably represented by SEQ ID NO. 46752, or a homolog or fragment thereof, or if the activity of a polypeptide encoded by a nucleic acid molecule comprising the nucleic acid SEQ ID NO. 46751, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 46751 or polypeptide SEQ ID NO. 46752, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical glycerol phosphate (lipid fraction). For example, an increase of the glycerol phosphate (lipid fraction) of at least 1 percent, particularly in a range of 18 to 82-percent is conferred as compared to a corresponding non-transformed wild type non-human organism.

Accordingly, in one embodiment, an increase of glycerol (polar fraction) in a non-human organism, as compared to a corresponding non-transformed wild type non-human organism, is conferred in the process of the invention, if the activity of a polypeptide showing the activity of a transcription factor, or if the activity of the polypeptide GM02LC21368, preferably represented by SEQ ID NO. 84199, or a homolog or fragment thereof, or if the activity of a polypeptide encoded by a nucleic acid molecule comprising the nucleic acid SEQ ID NO. 84198, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 84198 or polypeptide SEQ ID NO. 84199, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical glycerol (polar fraction). For example, an increase of the glycerol (polar fraction) of at least 1 percent, particularly in a range of 75 to 89-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a cyclin D, or if the activity of the polypeptide GM02LC44512, preferably represented by SEQ ID NO. 46851, or a homolog or fragment thereof, or if the activity of a polypeptide encoded by a nucleic acid molecule comprising the nucleic acid SEQ ID NO. 46850, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 46850 or polypeptide SEQ ID NO. 46851, respectively, or a homolog or a fragment thereof, is increased or generated, or if the activity cyclin D is increased or generated in a non-human organism, like a microorganism or a plant cell, plant or part thereof, especially with non-targeted localization, whereby the respective line disclose in column 7 the fine chemical glycerol phosphate (lipid fraction). For example, an increase of the glycerol phosphate (lipid fraction) of at least 1 percent, particularly in a range of 20 to 30-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a nitrate/nitrite transport protein, or if the activity of the polypeptide Sll1450, preferably represented by SEQ ID NO. 54338, or a homolog or fragment thereof, or if the activity of a polypeptide encoded by a nucleic acid molecule comprising the nucleic acid SEQ ID NO. 54337, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 54337 or polypeptide SEQ ID NO. 54338, respectively, or a homolog or a fragment thereof, is increased or generated, or if the activity nitrate/nitrite transport protein is increased or generated in a non-human organism, like a microorganism or a plant cell, plant or part thereof, especially with mitochondrial localization, whereby the respective line disclose in column 7 the fine chemical bis-glyceryl phosphate (polar fraction). For example, an increase of the bis-glyceryl phosphate (polar fraction) of at least 1 percent, particularly in a range of 41 to 70-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a carbamoyl-phosphate synthase subunit, or if the activity of the polypeptide Sll1498, preferably represented by SEQ ID NO. 95048, or a homolog or fragment thereof, or if the activity of a polypeptide encoded by a nucleic acid molecule comprising the nucleic acid SEQ ID NO. 95047, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 95047 or polypeptide SEQ ID NO. 95048, respectively, or a homolog or a fragment thereof, is increased or generated, or if the activity carbamoyl-phosphate synthase subunit is increased or generated in a non-human organism, like a microorganism or a plant cell, plant or part thereof, especially with non-targeted localization, whereby the respective line disclose in column 7 the fine chemical glycerol phosphate (lipid fraction). For example, an increase of the glycerol phosphate (lipid fraction) of at least 1 percent, particularly in a range of 18 to 32-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a CDP-diacylglycerol-glycerol-3-phosphate 3-phosphatidyltransferase, or if the activity of the polypeptide Sll1522, preferably represented by SEQ ID NO. 54453, or a homolog or fragment thereof, or if the activity of a polypeptide encoded by a nucleic acid molecule comprising the nucleic acid SEQ ID NO. 54452, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 54452 or polypeptide SEQ ID NO. 54453, respectively, or a homolog or a fragment thereof, is increased or generated, or if the activity CDP-diacylglycerol-glycerol-3-phosphate 3-phosphatidyltransferase is increased or generated in a non-human organism, like a microorganism or a plant cell, plant or part thereof, especially with plastidic localization, whereby the respective line disclose in column 7 the fine chemical glycerol (lipid fraction). For example, an increase of the glycerol (lipid fraction) of at least 1 percent, particularly in a range of 16 to 49-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a adenylate kinase, or if the activity of the polypeptide Sll1815, preferably represented by SEQ ID NO. 85809, or a homolog or fragment thereof, or if the activity of a polypeptide encoded by a nucleic acid molecule comprising the nucleic acid SEQ ID NO. 85808, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 85808 or polypeptide SEQ ID NO. 85809, respectively, or a homolog or a fragment thereof, is increased or generated, or if the activity adenylate kinase is increased or generated in a non-human organism, like a microorganism or a plant cell, plant or part thereof, especially with mitochondrial localization, whereby the respective line disclose in column 7 the fine chemical bis-glyceryl phosphate (polar fraction). For example, an increase of the bis-glyceryl phosphate (polar fraction) of at least 1 percent, particularly in a range of 49 to 68-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a acyl transferase, or if the activity of the polypeptide Sll1848, preferably represented by SEQ ID NO. 86448, or a homolog or fragment thereof, or if the activity of a polypeptide encoded by a nucleic acid molecule comprising the nucleic acid SEQ ID NO. 86447, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 86447 or polypeptide SEQ ID NO. 86448, respectively, or a homolog or a fragment thereof, is increased or generated, or if the activity acyl transferase is increased or generated in a non-human organism, like a microorganism or a plant cell, plant or part thereof, especially with plastidic localization, whereby the respective line disclose in column 7 the fine chemical glycerol (lipid fraction). For example, an increase of the glycerol (lipid fraction) of at least 1 percent, particularly in a range of 19 to 55-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a coproporphyrinogen III oxidase, or if the activity of the polypeptide Sll1917, preferably represented by SEQ ID NO. 11472, or a homolog or fragment thereof, or if the activity of a polypeptide encoded by a nucleic acid molecule comprising the nucleic acid SEQ ID NO. 11471, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 11471 or polypeptide SEQ ID NO. 11472, respectively, or a homolog or a fragment thereof, is increased or generated, or if the activity coproporphyrinogen III oxidase is increased or generated in a non-human organism, like a microorganism or a plant cell, plant or part thereof, especially with mitochondrial localization, whereby the respective line disclose in column 7 the fine chemical glycerol phosphate (lipid fraction). For example, an increase of the glycerol phosphate (lipid fraction) of at least 1 percent, particularly in a range of 17 to 67-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a oxidoreductase, or if the activity of the polypeptide Slr0338, preferably represented by SEQ ID NO. 11991, or a homolog or fragment thereof, or if the activity of a polypeptide encoded by a nucleic acid molecule comprising the nucleic acid SEQ ID NO. 11990, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 11990 or polypeptide SEQ ID NO. 11991, respectively, or a homolog or a fragment thereof, is increased or generated, or if the activity oxidoreductase is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 20 to 28-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a oxidoreductase, or if the activity of the polypeptide Slr0338, preferably represented by SEQ ID NO. 11991, or a homolog or fragment thereof, or if the activity of a polypeptide encoded by a nucleic acid molecule comprising the nucleic acid SEQ ID NO. 11990, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 11990 or polypeptide SEQ ID NO. 11991, respectively, or a homolog or a fragment thereof, is increased or generated, or if the activity oxidoreductase is increased or generated in a non-human organism, like a microorganism or a plant cell, plant or part thereof, especially with plastidic localization, whereby the respective line disclose in column 7 the fine chemical glycerol phosphate (lipid fraction). For example, an increase of the glycerol phosphate (lipid fraction) of at least 1 percent, particularly in a range of 18 to 39-percent is conferred as compared to a corresponding non-transformed wild type non-human organism.

Accordingly, in one embodiment, an increase of glycerol (polar fraction) in a non-human organism, as compared to a corresponding non-transformed wild type non-human organism, is conferred in the process of the invention, if the activity of a polypeptide showing the activity of a Mg-protoporphyrin IX monomethyl ester oxidative cyclase 66 kD subunit, or if the activity of the polypeptide Slr0905, preferably represented by SEQ ID NO. 147408, or a homolog or fragment thereof, or if the activity of a polypeptide encoded by a nucleic acid molecule comprising the nucleic acid SEQ ID NO. 147407, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 147407 or polypeptide SEQ ID NO. 147408, respectively, or a homolog or a fragment thereof, is increased or generated, or if the activity Mg-protoporphyrin IX monomethyl ester oxidative cyclase 66 kD subunit is increased or generated in a non-human organism, like a microorganism or a plant cell, plant or part thereof, especially with mitochondrial localization, whereby the respective line disclose in column 7 the fine chemical glycerol (polar fraction). For example, an increase of the glycerol (polar fraction) of at least 1 percent, particularly in a range of 53 to 73-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a dihydrolipoamide dehydrogenase, or if the activity of the polypeptide Slr1096, preferably represented by SEQ ID NO. 57735, or a homolog or fragment thereof, or if the activity of a polypeptide encoded by a nucleic acid molecule comprising the nucleic acid SEQ ID NO. 57734, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 57734 or polypeptide SEQ ID NO. 57735, respectively, or a homolog or a fragment thereof, is increased or generated, or if the activity dihydrolipoamide dehydrogenase is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 39 to 68-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a aromatic acid decarboxylase, or if the activity of the polypeptide Slr1099, preferably represented by SEQ ID NO. 116342, or a homolog or fragment thereof, or if the activity of a polypeptide encoded by a nucleic acid molecule comprising the nucleic acid SEQ ID NO. 116341, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 116341 or polypeptide SEQ ID NO. 116342, respectively, or a homolog or a fragment thereof, is increased or generated, or if the activity aromatic acid decarboxylase is increased or generated in a non-human organism, like a microorganism or a plant cell, plant or part thereof, especially with mitochondrial localization, whereby the respective line disclose in column 7 the fine chemical glycerol phosphate (lipid fraction). For example, an increase of the glycerol phosphate (lipid fraction) of at least 1 percent, particularly in a range of 17 to 29-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a phosphate import ATP-binding protein, or if the activity of the polypeptide Slr1250, preferably represented by SEQ ID NO. 116461, or a homolog or fragment thereof, or if the activity of a polypeptide encoded by a nucleic acid molecule comprising the nucleic acid SEQ ID NO. 116460, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 116460 or polypeptide SEQ ID NO. 116461, respectively, or a homolog or a fragment thereof, is increased or generated, or if the activity phosphate import ATP-binding protein is increased or generated in a non-human organism, like a microorganism or a plant cell, plant or part thereof, especially with mitochondrial localization, whereby the respective line disclose in column 7 the fine chemical glycerol phosphate (lipid fraction). For example, an increase of the glycerol phosphate (lipid fraction) of at least 1 percent, particularly in a range of 21 to 46-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a arginine decarboxylase, or if the activity of the polypeptide Slr1312, preferably represented by SEQ ID NO. 58325, or a homolog or fragment thereof, or if the activity of a polypeptide encoded by a nucleic acid molecule comprising the nucleic acid SEQ ID NO. 58324, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 58324 or polypeptide SEQ ID NO. 58325, respectively, or a homolog or a fragment thereof, is increased or generated, or if the activity arginine decarboxylase is increased or generated in a non-human organism, like a microorganism or a plant cell, plant or part thereof, especially with non-targeted localization, whereby the respective line disclose in column 7 the fine chemical glycerol phosphate (lipid fraction). For example, an increase of the glycerol phosphate (lipid fraction) of at least 1 percent, particularly in a range of 24 to 43-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a carbohydrate kinase, or if the activity of the polypeptide Slr1420, preferably represented by SEQ ID NO. 58591, or a homolog or fragment thereof, or if the activity of a polypeptide encoded by a nucleic acid molecule comprising the nucleic acid SEQ ID NO. 58590, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 58590 or polypeptide SEQ ID NO. 58591, respectively, or a homolog or a fragment thereof, is increased or generated, or if the activity carbohydrate kinase is increased or generated in a non-human organism, like a microorganism or a plant cell, plant or part thereof, especially with non-targeted localization, whereby the respective line disclose in column 7 the fine chemical glycerol phosphate (lipid fraction). For example, an increase of the glycerol phosphate (lipid fraction) of at least 1 percent, particularly in a range of 20 to 35-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a precorrin-8X methylmutase, or if the activity of the polypeptide Slr1467, preferably represented by SEQ ID NO. 154138, or a homolog or fragment thereof, or if the activity of a polypeptide encoded by a nucleic acid molecule comprising the nucleic acid SEQ ID NO. 154137, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 154137 or polypeptide SEQ ID NO. 154138, respectively, or a homolog or a fragment thereof, is increased or generated, or if the activity precorrin-8X methylmutase is increased or generated in a non-human organism, like a microorganism or a plant cell, plant or part thereof, especially with plastidic localization, whereby the respective line disclose in column 7 the fine chemical bis-glyceryl phosphate (polar fraction). For example, an increase of the bis-glyceryl phosphate (polar fraction) of at least 1 percent, particularly in a range of 57 to 97-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a NADH dehydrogenase, or if the activity of the polypeptide Slr1743, preferably represented by SEQ ID NO. 147458, or a homolog or fragment thereof, or if the activity of a polypeptide encoded by a nucleic acid molecule comprising the nucleic acid SEQ ID NO. 147457, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 147457 or polypeptide SEQ ID NO. 147458, respectively, or a homolog or a fragment thereof, is increased or generated, or if the activity NADH dehydrogenase is increased or generated in a non-human organism, like a microorganism or a plant cell, plant or part thereof, especially with plastidic localization, whereby the respective line disclose in column 7 the fine chemical bis-glyceryl phosphate (polar fraction). For example, an increase of the bis-glyceryl phosphate (polar fraction) of at least 1 percent, particularly in a range of 42 to 88-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a glutamate 5-kinase, or if the activity of the polypeptide Slr2035, preferably represented by SEQ ID NO. 127522, or a homolog or fragment thereof, or if the activity of a polypeptide encoded by a nucleic acid molecule comprising the nucleic acid SEQ ID NO. 127521, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 127521 or polypeptide SEQ ID NO. 127522, respectively, or a homolog or a fragment thereof, is increased or generated, or if the activity glutamate 5-kinase is increased or generated in a non-human organism, like a microorganism or a plant cell, plant or part thereof, especially with mitochondrial localization, whereby the respective line disclose in column 7 the fine chemical glycerol (lipid fraction). For example, an increase of the glycerol (lipid fraction) of at least 1 percent, particularly in a range of 18 to 22-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a threonine dehydratase, or if the activity of the polypeptide Slr2072, preferably represented by SEQ ID NO. 12342, or a homolog or fragment thereof, or if the activity of a polypeptide encoded by a nucleic acid molecule comprising the nucleic acid SEQ ID NO. 12341, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 12341 or polypeptide SEQ ID NO. 12342, respectively, or a homolog or a fragment thereof, is increased or generated, or if the activity threonine dehydratase is increased or generated in a non-human organism, like a microorganism or a plant cell, plant or part thereof, especially with mitochondrial localization, whereby the respective line disclose in column 7 the fine chemical glycerol (lipid fraction). For example, an increase of the glycerol (lipid fraction) of at least 1 percent, particularly in a range of 18 to 43-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a threonine dehydratase, or if the activity of the polypeptide Slr2072, preferably represented by SEQ ID NO. 12342, or a homolog or fragment thereof, or if the activity of a polypeptide encoded by a nucleic acid molecule comprising the nucleic acid SEQ ID NO. 12341, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 12341 or polypeptide SEQ ID NO. 12342, respectively, or a homolog or a fragment thereof, is increased or generated, or if the activity threonine dehydratase is increased or generated in a non-human organism, like a microorganism or a plant cell, plant or part thereof, especially with mitochondrial localization, whereby the respective line disclose in column 7 the fine chemical glycerol phosphate (lipid fraction). For example, an increase of the glycerol phosphate (lipid fraction) of at least 1 percent, particularly in a range of 19 to 28-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a photosystem I reaction centre subunit, or if the activity of the polypeptide Smr0004, preferably represented by SEQ ID NO. 136908, or a homolog or fragment thereof, or if the activity of a polypeptide encoded by a nucleic acid molecule comprising the nucleic acid SEQ ID NO. 136907, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 136907 or polypeptide SEQ ID NO. 136908, respectively, or a homolog or a fragment thereof, is increased or generated, or if the activity photosystem I reaction centre subunit is increased or generated in a non-human organism, like a microorganism or a plant cell, plant or part thereof, especially with plastidic localization, whereby the respective line disclose in column 7 the fine chemical bis-glyceryl phosphate (polar fraction). For example, an increase of the bis-glyceryl phosphate (polar fraction) of at least 1 percent, particularly in a range of 44 to 60-percent is conferred as compared to a corresponding non-transformed wild type non-human organism.

Accordingly, in one embodiment, an increase of triacylglycerol in a non-human organism, as compared to a corresponding non-transformed wild type non-human organism, is conferred in the process of the invention, if the activity of a polypeptide showing the activity of a cell division protein, or if the activity of the polypeptide TTC0035, preferably represented by SEQ ID NO. 60302, or a homolog or fragment thereof, or if the activity of a polypeptide encoded by a nucleic acid molecule comprising the nucleic acid SEQ ID NO. 60301, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 60301 or polypeptide SEQ ID NO. 60302, respectively, or a homolog or a fragment thereof, is increased or generated, or if the activity cell division protein is increased or generated in a non-human organism, like a microorganism or a plant cell, plant or part thereof, especially with non-targeted localization, whereby the respective line disclose in column 7 the fine chemical triacylglycerol. For example, an increase of the triacylglycerol of at least 1 percent, particularly in a range of 73 to 163-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a metal-dependent hydrolase, or if the activity of the polypeptide TTC0917, preferably represented by SEQ ID NO. 61554, or a homolog or fragment thereof, or if the activity of a polypeptide encoded by a nucleic acid molecule comprising the nucleic acid SEQ ID NO. 61553, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 61553 or polypeptide SEQ ID NO. 61554, respectively, or a homolog or a fragment thereof, is increased or generated, or if the activity metal-dependent hydrolase is increased or generated in a non-human organism, like a microorganism or a plant cell, plant or part thereof, especially with non-targeted localization, whereby the respective line disclose in column 7 the fine chemical glycerol phosphate (lipid fraction). For example, an increase of the glycerol phosphate (lipid fraction) of at least 1 percent, particularly in a range of 21 to 73-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a TTC1386-protein, or if the activity of the polypeptide TTC1386, preferably represented by SEQ ID NO. 62080, or a homolog or fragment thereof, or if the activity of a polypeptide encoded by a nucleic acid molecule comprising the nucleic acid SEQ ID NO. 62079, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 62079 or polypeptide SEQ ID NO. 62080, respectively, or a homolog or a fragment thereof, is increased or generated, or if the activity TTC1386-protein is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 23 to 34-percent is conferred as compared to a corresponding non-transformed wild type non-human organism.

Accordingly, in one embodiment, an increase of glycerol-3-phosphate (polar fraction) in a non-human organism, as compared to a corresponding non-transformed wild type non-human organism, is conferred in the process of the invention, if the activity of a polypeptide showing the activity of a YAL013W-protein, or if the activity of the polypeptide Yal013w, preferably represented by SEQ ID NO. 108093, or a homolog or fragment thereof, or if the activity of a polypeptide encoded by a nucleic acid molecule comprising the nucleic acid SEQ ID NO. 108092, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 108092 or polypeptide SEQ ID NO. 108093, respectively, or a homolog or a fragment thereof, is increased or generated, or if the activity YAL013W-protein is increased or generated in a non-human organism, like a microorganism or a plant cell, plant or part thereof, especially with non-targeted localization, whereby the respective line disclose in column 7 the fine chemical glycerol-3-phosphate (polar fraction). For example, an increase of the glycerol-3-phosphate (polar fraction) of at least 1 percent, particularly in a range of 136 to 212-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a transcription factor, or if the activity of the polypeptide Ybl021c, preferably represented by SEQ ID NO. 62525, or a homolog or fragment thereof, or if the activity of a polypeptide encoded by a nucleic acid molecule comprising the nucleic acid SEQ ID NO. 62524, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 62524 or polypeptide SEQ ID NO. 62525, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 17 to 23-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a acid phosphatase precursor, or if the activity of the polypeptide Ybr092c, preferably represented by SEQ ID NO. 119348, or a homolog or fragment thereof, or if the activity of a polypeptide encoded by a nucleic acid molecule comprising the nucleic acid SEQ ID NO. 119347, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 119347 or polypeptide SEQ ID NO. 119348, respectively, or a homolog or a fragment thereof, is increased or generated, or if the activity acid phosphatase precursor is increased or generated in a non-human organism, like a microorganism or a plant cell, plant or part thereof, especially with plastidic localization, whereby the respective line disclose in column 7 the fine chemical glycerol phosphate (lipid fraction). For example, an increase of the glycerol phosphate (lipid fraction) of at least 1 percent, particularly in a range of 24 to 31-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a cell division control protein, or if the activity of the polypeptide Ybr160w, preferably represented by SEQ ID NO. 62718, or a homolog or fragment thereof, or if the activity of a polypeptide encoded by a nucleic acid molecule comprising the nucleic acid SEQ ID NO. 62717, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 62717 or polypeptide SEQ ID NO. 62718, respectively, or a homolog or a fragment thereof, is increased or generated, or if the activity cell division control protein is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 17 to 63-percent is conferred as compared to a corresponding non-transformed wild type non-human organism.

Accordingly, in one embodiment, an increase of glycerol (polar fraction) in a non-human organism, as compared to a corresponding non-transformed wild type non-human organism, is conferred in the process of the invention, if the activity of a polypeptide showing the activity of a cell division control protein, or if the activity of the polypeptide Ybr160w, preferably represented by SEQ ID NO. 62718, or a homolog or fragment thereof, or if the activity of a polypeptide encoded by a nucleic acid molecule comprising the nucleic acid SEQ ID NO. 62717, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 62717 or polypeptide SEQ ID NO. 62718, respectively, or a homolog or a fragment thereof, is increased or generated, or if the activity cell division control protein is increased or generated in a non-human organism, like a microorganism or a plant cell, plant or part thereof, especially with non-targeted localization, whereby the respective line disclose in column 7 the fine chemical glycerol (polar fraction). For example, an increase of the glycerol (polar fraction) of at least 1 percent, particularly in a range of 54 to 85-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a ycr102c-protein, or if the activity of the polypeptide Ycr102c, preferably represented by SEQ ID NO. 63265, or a homolog or fragment thereof, or if the activity of a polypeptide encoded by a nucleic acid molecule comprising the nucleic acid SEQ ID NO. 63264, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 63264 or polypeptide SEQ ID NO. 63265, respectively, or a homolog or a fragment thereof, is increased or generated, or if the activity ycr102c-protein is increased or generated in a non-human organism, like a microorganism or a plant cell, plant or part thereof, especially with plastidic localization, whereby the respective line disclose in column 7 the fine chemical bis-glyceryl phosphate (polar fraction). For example, an increase of the bis-glyceryl phosphate (polar fraction) of at least 1 percent, particularly in a range of 42 to 81-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a ydr273w-protein, or if the activity of the polypeptide Ydr273w, preferably represented by SEQ ID NO. 90160, or a homolog or fragment thereof, or if the activity of a polypeptide encoded by a nucleic acid molecule comprising the nucleic acid SEQ ID NO. 90159, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 90159 or polypeptide SEQ ID NO. 90160, respectively, or a homolog or a fragment thereof, is increased or generated, or if the activity ydr273w-protein is increased or generated in a non-human organism, like a microorganism or a plant cell, plant or part thereof, especially with plastidic localization, whereby the respective line disclose in column 7 the fine chemical bis-glyceryl phosphate (polar fraction). For example, an increase of the bis-glyceryl phosphate (polar fraction) of at least 1 percent, particularly in a range of 41 to 48-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a molecular chaperone portein, or if the activity of the polypeptide Yfl016c, preferably represented by SEQ ID NO. 63808, or a homolog or fragment thereof, or if the activity of a polypeptide encoded by a nucleic acid molecule comprising the nucleic acid SEQ ID NO. 63807, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 63807 or polypeptide SEQ ID NO. 63808, respectively, or a homolog or a fragment thereof, is increased or generated, or if the activity molecular chaperone portein is increased or generated in a non-human organism, like a microorganism or a plant cell, plant or part thereof, especially with non-targeted localization, whereby the respective line disclose in column 7 the fine chemical glycerol phosphate (lipid fraction). For example, an increase of the glycerol phosphate (lipid fraction) of at least 1 percent, particularly in a range of 22 to 31-percent is conferred as compared to a corresponding non-transformed wild type non-human organism.

Accordingly, in one embodiment, an increase of triacylglycerol in a non-human organism, as compared to a corresponding non-transformed wild type non-human organism, is conferred in the process of the invention, if the activity of a polypeptide showing the activity of a YFL019C-protein, or if the activity of the polypeptide Yfl019c, preferably represented by SEQ ID NO. 64145, or a homolog or fragment thereof, or if the activity of a polypeptide encoded by a nucleic acid molecule comprising the nucleic acid SEQ ID NO. 64144, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 64144 or polypeptide SEQ ID NO. 64145, respectively, or a homolog or a fragment thereof, is increased or generated, or if the activity YFL019C-protein is increased or generated in a non-human organism, like a microorganism or a plant cell, plant or part thereof, especially with non-targeted localization, whereby the respective line disclose in column 7 the fine chemical triacylglycerol. For example, an increase of the triacylglycerol of at least 1 percent, particularly in a range of 36 to 110-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a alcohol dehydrogenase, or if the activity of the polypeptide Ygl256w, preferably represented by SEQ ID NO. 130743, or a homolog or fragment thereof, or if the activity of a polypeptide encoded by a nucleic acid molecule comprising the nucleic acid SEQ ID NO. 130742, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 130742 or polypeptide SEQ ID NO. 130743, respectively, or a homolog or a fragment thereof, is increased or generated, or if the activity alcohol dehydrogenase is increased or generated in a non-human organism, like a microorganism or a plant cell, plant or part thereof, especially with plastidic localization, whereby the respective line disclose in column 7 the fine chemical bis-glyceryl phosphate (polar fraction). For example, an increase of the bis-glyceryl phosphate (polar fraction) of at least 1 percent, particularly in a range of 42 to 93-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a biotin synthase, or if the activity of the polypeptide Ygr286c, preferably represented by SEQ ID NO. 128656, or a homolog or fragment thereof, or if the activity of a polypeptide encoded by a nucleic acid molecule comprising the nucleic acid SEQ ID NO. 128655, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 128655 or polypeptide SEQ ID NO. 128656, respectively, or a homolog or a fragment thereof, is increased or generated, or if the activity biotin synthase is increased or generated in a non-human organism, like a microorganism or a plant cell, plant or part thereof, especially with plastidic localization, whereby the respective line disclose in column 7 the fine chemical glycerol (lipid fraction).

For example, an increase of the glycerol (lipid fraction) of at least 1 percent, particularly in a range of 16 to 43-percent is conferred as compared to a corresponding non-transformed wild type non-human organism.

Accordingly, in one embodiment, an increase of triacylglycerol in a non-human organism, as compared to a corresponding non-transformed wild type non-human organism, is conferred in the process of the invention, if the activity of a polypeptide showing the activity of a mitochondrial processing protease, or if the activity of the polypeptide Yhr024c, preferably represented by SEQ ID NO. 64337, or a homolog or fragment thereof, or if the activity of a polypeptide encoded by a nucleic acid molecule comprising the nucleic acid SEQ ID NO. 64336, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 64336 or polypeptide SEQ ID NO. 64337, respectively, or a homolog or a fragment thereof, is increased or generated, or if the activity mitochondrial processing protease is increased or generated in a non-human organism, like a microorganism or a plant cell, plant or part thereof, especially with plastidic localization, whereby the respective line disclose in column 7 the fine chemical triacylglycerol. For example, an increase of the triacylglycerol of at least 1 percent, particularly in a range of 52 to 138-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a YHR202W-protein, or if the activity of the polypeptide Yhr202w, preferably represented by SEQ ID NO. 154395, or a homolog or fragment thereof, or if the activity of a polypeptide encoded by a nucleic acid molecule comprising the nucleic acid SEQ ID NO. 154394, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 154394 or polypeptide SEQ ID NO. 154395, respectively, or a homolog or a fragment thereof, is increased or generated, or if the activity YHR202W-protein is increased or generated in a non-human organism, like a microorganism or a plant cell, plant or part thereof, especially with plastidic localization, whereby the respective line disclose in column 7 the fine chemical bis-glyceryl phosphate (polar fraction). For example, an increase of the bis-glyceryl phosphate (polar fraction) of at least 1 percent, particularly in a range of 36 to 46-percent is conferred as compared to a corresponding non-transformed wild type non-human organism.

Accordingly, in one embodiment, an increase of glycerol (polar fraction) in a non-human organism, as compared to a corresponding non-transformed wild type non-human organism, is conferred in the process of the invention, if the activity of a polypeptide showing the activity of a (DL)-glycerol-3-phosphatase, or if the activity of the polypeptide Yil053w, preferably represented by SEQ ID NO. 14770, or a homolog or fragment thereof, or if the activity of a polypeptide encoded by a nucleic acid molecule comprising the nucleic acid SEQ ID NO. 14769, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 14769 or polypeptide SEQ ID NO. 14770, respectively, or a homolog or a fragment thereof, is increased or generated, or if the activity (DL)-glycerol-3-phosphatase is increased or generated in a non-human organism, like a microorganism or a plant cell, plant or part thereof, especially with non-targeted localization, whereby the respective line disclose in column 7 the fine chemical glycerol (polar fraction). For example, an increase of the glycerol (polar fraction) of at least 1 percent, particularly in a range of 49 to 2038-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a YIL083C-protein, or if the activity of the polypeptide Yil083c, preferably represented by SEQ ID NO. 149455, or a homolog or fragment thereof, or if the activity of a polypeptide encoded by a nucleic acid molecule comprising the nucleic acid SEQ ID NO. 149454, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 149454 or polypeptide SEQ ID NO. 149455, respectively, or a homolog or a fragment thereof, is increased or generated, or if the activity YIL083C-protein is increased or generated in a non-human organism, like a microorganism or a plant cell, plant or part thereof, especially with plastidic localization, whereby the respective line disclose in column 7 the fine chemical bis-glyceryl phosphate (polar fraction). For example, an increase of the bis-glyceryl phosphate (polar fraction) of at least 1 percent, particularly in a range of 48 to 101-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a YJL062W-A-protein, or if the activity of the polypeptide Yjl062w-a, preferably represented by SEQ ID NO. 154438, or a homolog or fragment thereof, or if the activity of a polypeptide encoded by a nucleic acid molecule comprising the nucleic acid SEQ ID NO. 154437, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 154437 or polypeptide SEQ ID NO. 154438, respectively, or a homolog or a fragment thereof, is increased or generated, or if the activity YJL062W-A-protein is increased or generated in a non-human organism, like a microorganism or a plant cell, plant or part thereof, especially with non-targeted localization, whereby the respective line disclose in column 7 the fine chemical glycerol phosphate (lipid fraction). For example, an increase of the glycerol phosphate (lipid fraction) of at least 1 percent, particularly in a range of 18 to 23-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a DnaJ-like chaperone, or if the activity of the polypeptide Yjl073w, preferably represented by SEQ ID NO. 64965, or a homolog or fragment thereof, or if the activity of a polypeptide encoded by a nucleic acid molecule comprising the nucleic acid SEQ ID NO. 64964, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 64964 or polypeptide SEQ ID NO. 64965, respectively, or a homolog or a fragment thereof, is increased or generated, or if the activity DnaJ-like chaperone is increased or generated in a non-human organism, like a microorganism or a plant cell, plant or part thereof, especially with plastidic localization, whereby the respective line disclose in column 7 the fine chemical bis-glyceryl phosphate (polar fraction). For example, an increase of the bis-glyceryl phosphate (polar fraction) of at least 1 percent, particularly in a range of 33 to 36-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a polygalacturonase, or if the activity of the polypeptide Yjr153w, preferably represented by SEQ ID NO. 66275, or a homolog or fragment thereof, or if the activity of a polypeptide encoded by a nucleic acid molecule comprising the nucleic acid SEQ ID NO. 66274, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 66274 or polypeptide SEQ ID NO. 66275, respectively, or a homolog or a fragment thereof, is increased or generated, or if the activity polygalacturonase is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 45 to 131-percent is conferred as compared to a corresponding non-transformed wild type non-human organism.

Accordingly, in one embodiment, an increase of glycerol (polar fraction) in a non-human organism, as compared to a corresponding non-transformed wild type non-human organism, is conferred in the process of the invention, if the activity of a polypeptide showing the activity of a vacuolar sorting protein, or if the activity of the polypeptide Ylr025w, preferably represented by SEQ ID NO. 150604, or a homolog or fragment thereof, or if the activity of a polypeptide encoded by a nucleic acid molecule comprising the nucleic acid SEQ ID NO. 150603, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 150603 or polypeptide SEQ ID NO. 150604, respectively, or a homolog or a fragment thereof, is increased or generated, or if the activity vacuolar sorting protein is increased or generated in a non-human organism, like a microorganism or a plant cell, plant or part thereof, especially with non-targeted localization, whereby the respective line disclose in column 7 the fine chemical glycerol (polar fraction). For example, an increase of the glycerol (polar fraction) of at least 1 percent, particularly in a range of 62 to 120-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a YLR099C-protein, or if the activity of the polypeptide Ylr099c, preferably represented by SEQ ID NO. 154455, or a homolog or fragment thereof, or if the activity of a polypeptide encoded by a nucleic acid molecule comprising the nucleic acid SEQ ID NO. 154454, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 154454 or polypeptide SEQ ID NO. 154455, respectively, or a homolog or a fragment thereof, is increased or generated, or if the activity YLR099C-protein is increased or generated in a non-human organism, like a microorganism or a plant cell, plant or part thereof, especially with plastidic localization, whereby the respective line disclose in column 7 the fine chemical bis-glyceryl phosphate (polar fraction). For example, an increase of the bis-glyceryl phosphate (polar fraction) of at least 1 percent, particularly in a range of 297 to 648-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a cyclin, or if the activity of the polypeptide Ylr210w, preferably represented by SEQ ID NO. 129066, or a homolog or fragment thereof, or if the activity of a polypeptide encoded by a nucleic acid molecule comprising the nucleic acid SEQ ID NO. 129065, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 129065 or polypeptide SEQ ID NO. 129066, respectively, or a homolog or a fragment thereof, is increased or generated, or if the activity cyclin is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 17 to 25-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a ylr326w-protein, or if the activity of the polypeptide Ylr326w, preferably represented by SEQ ID NO. 129124, or a homolog or fragment thereof, or if the activity of a polypeptide encoded by a nucleic acid molecule comprising the nucleic acid SEQ ID NO. 129123, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 129123 or polypeptide SEQ ID NO. 129124, respectively, or a homolog or a fragment thereof, is increased or generated, or if the activity ylr326w-protein is increased or generated in a non-human organism, like a microorganism or a plant cell, plant or part thereof, especially with plastidic localization, whereby the respective line disclose in column 7 the fine chemical glycerol (lipid fraction). For example, an increase of the glycerol (lipid fraction) of at least 1 percent, particularly in a range of 17 to 42-percent is conferred as compared to a corresponding non-transformed wild type non-human organism.

Accordingly, in one embodiment, an increase of glycerol phosphate (lipid fraction) in a non-human organism, as compared to a corresponding non-transformed wild type non-human organism, is conferred in the process of the invention, if the activity of a polypeptide showing the activity of a ylr326w-protein, or if the activity of the polypeptide Ylr326w, preferably represented by SEQ ID NO. 129124, or a homolog or fragment thereof, or if the activity of a polypeptide encoded by a nucleic acid molecule comprising the nucleic acid SEQ ID NO. 129123, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 129123 or polypeptide SEQ ID NO. 129124, respectively, or a homolog or a fragment thereof, is increased or generated, or if the activity ylr326w-protein is increased or generated in a non-human organism, like a microorganism or a plant cell, plant or part thereof, especially with plastidic localization, whereby the respective line disclose in column 7 the fine chemical glycerol phosphate (lipid fraction). For example, an increase of the glycerol phosphate (lipid fraction) of at least 1 percent, particularly in a range of 26 to 46-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a heat shock protein Hsp40, or if the activity of the polypeptide Ynl007c, preferably represented by SEQ ID NO. 139174, or a homolog or fragment thereof, or if the activity of a polypeptide encoded by a nucleic acid molecule comprising the nucleic acid SEQ ID NO. 139173, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 139173 or polypeptide SEQ ID NO. 139174, respectively, or a homolog or a fragment thereof, is increased or generated, or if the activity heat shock protein Hsp40 is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 17 to 57-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a phosphotransferase, or if the activity of the polypeptide Ynl130c, preferably represented by SEQ ID NO. 129471, or a homolog or fragment thereof, or if the activity of a polypeptide encoded by a nucleic acid molecule comprising the nucleic acid SEQ ID NO. 129470, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 129470 or polypeptide SEQ ID NO. 129471, respectively, or a homolog or a fragment thereof, is increased or generated, or if the activity phosphotransferase is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 17 to 53-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a Elongation factor Tu, or if the activity of the polypeptide Yor187w, preferably represented by SEQ ID NO. 129575, or a homolog or fragment thereof, or if the activity of a polypeptide encoded by a nucleic acid molecule comprising the nucleic acid SEQ ID NO. 129574, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 129574 or polypeptide SEQ ID NO. 129575, respectively, or a homolog or a fragment thereof, is increased or generated, or if the activity Elongation factor Tu is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, partitularly in a range of 18 to 49-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a YPL162C.protein, or if the activity of the polypeptide Ypl162c, preferably represented by SEQ ID NO. 154478, or a homolog or fragment thereof, or if the activity of a polypeptide encoded by a nucleic acid molecule comprising the nucleic acid SEQ ID NO. 154477, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 154477 or polypeptide SEQ ID NO. 154478, respectively, or a homolog or a fragment thereof, is increased or generated, or if the activity YPL162C.protein is increased or generated in a non-human organism, like a microorganism or a plant cell, plant or part thereof, especially with plastidic localization, whereby the respective line disclose in column 7 the fine chemical bis-glyceryl phosphate (polar fraction). For example, an increase of the bis-glyceryl phosphate (polar fraction) of at least 1 percent, particularly in a range of 37 to 52-percent is conferred as compared to a corresponding non-transformed wild type non-human organism.

Accordingly, in one embodiment, an increase of glycerol (lipid fraction) in a non-human organism, as compared to a corresponding non-transformed wild type non-human organism, is conferred in the process of the invention, if the activity of a polypeptide showing the activity of a mitogen-activated protein kinase, or if the activity of the polypeptide Ypr054w, preferably represented by SEQ ID NO. 140111, or a homolog or fragment thereof, or if the activity of a polypeptide encoded by a nucleic acid molecule comprising the nucleic acid SEQ ID NO. 140110, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 140110 or polypeptide SEQ ID NO. 140111, respectively, or a homolog or a fragment thereof, is increased or generated, or if the activity mitogen-activated protein kinase is increased or generated in a non-human organism, like a microorganism or a plant cell, plant or part thereof, especially with non-targeted localization, whereby the respective line disclose in column 7 the fine chemical glycerol (lipid fraction). For example, an increase of the glycerol (lipid fraction) of at least 1 percent, particularly in a range of 20 to 42-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a Zm_{—}4842_BE510522-protein, or if the activity of the polypeptide Zm_{—}4842_BE510522, preferably represented by SEQ ID NO. 68414, or a homolog or fragment thereof, or if the activity of a polypeptide encoded by a nucleic acid molecule comprising the nucleic acid SEQ ID NO. 68413, preferably the coding region thereof, or a homolog or fragment thereof, e.g. derived from Zea mays, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 68413 or polypeptide SEQ ID NO. 68414, respectively, or a homolog or a fragment thereof, is increased or generated, or if the activity Zm_{—}4842_BE510522-protein is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 46 to 72-percent is conferred as compared to a corresponding non-transformed wild type non-human organism.

Accordingly, in one embodiment, an increase of bis-glyceryl phosphate (polar fraction) in a non-human organism, as compared to a corresponding non-transformed wild type non-human organism, is conferred in the process of the invention, if the activity of a polypeptide showing the activity of a 49747384_SOYBEAN-protein, or if the activity of the polypeptide 49747384_SOYBEAN, preferably represented by SEQ ID NO. 70, or a homolog or fragment thereof, or if the activity of a polypeptide encoded by a nucleic acid molecule comprising the nucleic acid SEQ ID NO. 69, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 19, column 5 or 8 in the respective same line as the nucleic acid molecule SEQ ID NO. 69 or polypeptide SEQ ID NO. 70, respectively, or a homolog or a fragment thereof, is increased or generated, or if the activity 49747384_SOYBEAN-protein is increased or generated in a non-human organism, like a microorganism or a plant cell, plant or part thereof, especially with non-targeted localization, whereby the respective line disclose in column 7 the fine chemical bis-glyceryl phosphate (polar fraction). For example, an increase of the bis-glyceryl phosphate (polar fraction) of at least 1 percent, particularly in a range of 47 to 114-percent is conferred as compared to a corresponding non-transformed wild type non-human organism.

[0096.1.7.19] to [0103.1.7.19] for the disclosure of theses paragraphs see [0096.1.7.7] to [0103.1.7.7] above

In this paragraph “A” means “Goffeau et al., Science 274 (5287), 546 (1996)”, “B” means “Blattner et al., Science 277 (5331), 1453 (1997)”, “C” means “Nakamura et al., Nucleic Acids Res. 28(1), 72 (2000)”, “D” means “deposition of the sequences to the EMBL/GenBank/DDBJ databases under “Sequencing of the draft genome assembly of Azotobacter vinelandii AvOP”. in June 2005 by Copeland et al.”, “E” means “Henne et al., Nat. Biotechnol. 22 (5), 547 (2004)”, “F” means “Kaul et al., Nature 408 (6814), 796 (2000)”, and “G” means “Matsumoto et al., Nature 436 (7052), 793 (2005)”.

The nucleic acid sequence of At1g07430 from Arabidopsis thaliana, e.g. as shown in the respective line in column 5 of Table I, application no. 19, has been published in F. And the activity of the gene product thereof is the activity of protein phosphatase.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said At1g07430, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said At1g07430, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said At1g07430, or a functional equivalent or a homolog thereof as depicted in column 8 of Table II, application no. 19, preferably a homolog or functional equivalent as depicted in column 8 of Table II B, application no. 19, and being depicted in the same respective line as said At1g07430, and preferably the activity is increased non-targeted, whereby the respective line disclose in column 7 the fine chemical glycerol phosphate (lipid fraction).

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 17451, preferably the coding region thereof, conferred the production of or the increase in glycerol phosphate (lipid fraction) compared with the wild type control.

The nucleic acid sequence of At1g23110 from Arabidopsis thaliana, e.g. as shown in the respective line in column 5 of Table I, application no. 19, has been published in F. And the activity of the gene product thereof is the activity of At1g23110-protein.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “At1g23110-protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said At1g23110, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said At1g23110, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said At1g23110, or a functional equivalent or a homolog thereof as depicted in column 8 of Table II, application no. 19, preferably a homolog or functional equivalent as depicted in column 8 of Table II B, application no. 19, and being depicted in the same respective line as said At1g23110, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “At1g23110-protein”, preferably it is the molecule of section (a) or (b) of this paragraph.

In particular, it was observed that in plants, especially in Arabidopsis thaliana, increasing or generating the activity of a gene product non-targeted with the activity of a “At1g23110-protein”, preferably being encoded by a gene comprising the nucleic acid sequence SEQ ID NO. 102086, preferably the coding region thereof, conferred the production of or the increase in bis-glyceryl phosphate (polar fraction) compared with the wild type control.

The nucleic acid sequence of At1g36730 from Arabidopsis thaliana, e.g. as shown in the respective line in column 5 of Table I, application no. 19, has been published in F. And the activity of the gene product thereof is the activity of eukaryotic translation initiation factor 5.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “eukaryotic translation initiation factor 5”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said At1g36730, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said At1g36730, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said At1g36730, or a functional equivalent or a homolog thereof as depicted in column 8 of Table II, application no. 19, preferably a homolog or functional equivalent as depicted in column 8 of Table II B, application no. 19, and being depicted in the same respective line as said At1g36730, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “eukaryotic translation initiation factor 5”, preferably it is the molecule of section (a) or (b) of this paragraph.

In particular, it was observed that in plants, especially in Arabidopsis thaliana, increasing or generating the activity of a gene product non-targeted with the activity of a “eukaryotic translation initiation factor 5”, preferably being encoded by a gene comprising the nucleic acid sequence SEQ ID NO. 17968, preferably the coding region thereof, conferred the production of or the increase in bis-glyceryl phosphate (polar fraction) compared with the wild type control.

The nucleic acid sequence of At1g36730 from Arabidopsis thaliana, e.g. as shown in the respective line in column 5 of Table I, application no. 19, has been published in F. And the activity of the gene product thereof is the activity of eukaryotic translation initiation factor 5.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “eukaryotic translation initiation factor 5”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said At1g36730, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said At1g36730, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said At1g36730, or a functional equivalent or a homolog thereof as depicted in column 8 of Table II, application no. 19, preferably a homolog or functional equivalent as depicted in column 8 of Table II B, application no. 19, and being depicted in the same respective line as said At1g36730, and preferably the activity is increased non-targeted, whereby the respective line disclose in column 7 the fine chemical glycerol phosphate (lipid fraction).

Accordingly, in one embodiment, the molecule which activity is to be increased in the process of the invention is the gene product with an activity as a “eukaryotic translation initiation factor 5”, preferably it is the molecule of section (a) or (b) of this paragraph.

In particular, it was observed that in plants, especially in Arabidopsis thaliana, increasing or generating the activity of a gene product non-targeted with the activity of a “eukaryotic translation initiation factor 5”, preferably being encoded by a gene comprising the nucleic acid sequence SEQ ID NO. 17968, preferably the coding region thereof, conferred the production of or the increase in glycerol phosphate (lipid fraction) compared with the wild type control.

The nucleic acid sequence of At1g67340 from Arabidopsis thaliana, e.g. as shown in the respective line in column 5 of Table I, application no. 19, has been published in F. And the activity of the gene product thereof is the activity of At1g67340-protein.

Accordingly, in one embodiment, the process of the present invention for producing triacylglycerol in a non-human organism, like a microorganism or a plant or a part thereof, comprises increasing or generating the activity of a gene product with the activity of a gene product conferring the activity of “At1g67340-protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical triacylglycerol), application no. 19, preferably the coding region thereof, or a homolog or a fragment thereof, and being depicted in the same respective line as said At1g67340, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said At1g67340, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said At1g67340, or a functional equivalent or a homolog thereof as depicted in column 8 of Table II, application no. 19, preferably a homolog or functional equivalent as depicted in column 8 of Table II B, application no. 19, and being depicted in the same respective line as said At1g67340, and preferably the activity is increased non-targeted, whereby the respective line disclose in column 7 the fine chemical triacylglycerol.

Accordingly, in one embodiment, the molecule which activity is to be increased in the process of the invention is the gene product with an activity as a “At1g67340-protein”, preferably it is the molecule of section (a) or (b) of this paragraph.

In particular, it was observed that in plants, especially in Arabidopsis thaliana, increasing or generating the activity of a gene product non-targeted with the activity of a “At1g67340-protein”, preferably being encoded by a gene comprising the nucleic acid sequence SEQ ID NO. 19364, preferably the coding region thereof, conferred the production of or the increase in triacylglycerol compared with the wild type control.

The nucleic acid sequence of At1g68320 from Arabidopsis thaliana, e.g. as shown in the respective line in column 5 of Table I, application no. 19, has been published in F. And the activity of the gene product thereof is the activity of transcription factor.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said At1g68320, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said At1g68320, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said At1g68320, or a functional equivalent or a homolog thereof as depicted in column 8 of Table II, application no. 19, preferably a homolog or functional equivalent as depicted in column 8 of Table II B, application no. 19, and being depicted in the same respective line as said At1g68320, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 1061, preferably the coding region thereof, conferred the production of or the increase in glycerol (lipid fraction) compared with the wild type control.

The nucleic acid sequence of At1g72770 from Arabidopsis thaliana, e.g. as shown in the respective line in column 5 of Table I, application no. 19, has been published in F. And the activity of the gene product thereof is the activity of protein phosphatase.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said At1g72770, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said At1g72770, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said At1g72770, or a functional equivalent or a homolog thereof as depicted in column 8 of Table II, application no. 19, preferably a homolog or functional equivalent as depicted in column 8 of Table II B, application no. 19, and being depicted in the same respective line as said At1g72770, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19419, preferably the coding region thereof, conferred the production of or the increase in bis-glyceryl phosphate (polar fraction) compared with the wild type control.

The nucleic acid sequence of At1g72770 from Arabidopsis thaliana, e.g. as shown in the respective line in column 5 of Table I, application no. 19, has been published in F. And the activity of the gene product thereof is the activity of protein phosphatase.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said At1g72770, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said At1g72770, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said At1g72770, or a functional equivalent or a homolog thereof as depicted in column 8 of Table II, application no. 19, preferably a homolog or functional equivalent as depicted in column 8 of Table II B, application no. 19, and being depicted in the same respective line as said At1g72770, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19419, preferably the coding region thereof, conferred the production of or the increase in glycerol (lipid fraction) compared with the wild type control.

The nucleic acid sequence of At1g72770 from Arabidopsis thaliana, e.g. as shown in the respective line in column 5 of Table I, application no. 19, has been published in F. And the activity of the gene product thereof is the activity of protein phosphatase.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said At1g72770, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said At1g72770, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said At1g72770, or a functional equivalent or a homolog thereof as depicted in column 8 of Table II, application no. 19, preferably a homolog or functional equivalent as depicted in column 8 of Table II B, application no. 19, and being depicted in the same respective line as said At1g72770, and preferably the activity is increased non-targeted, whereby the respective line disclose in column 7 the fine chemical glycerol phosphate (lipid fraction).

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 19419, preferably the coding region thereof, conferred the production of or the increase in glycerol phosphate (lipid fraction) compared with the wild type control.

The nucleic acid sequence of At2g30360 from Arabidopsis thaliana, e.g. as shown in the respective line in column 5 of Table I, application no. 19, has been published in F. And the activity of the gene product thereof is the activity of CBL-interacting protein kinase.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “CBL-interacting protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said At2g30360, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said At2g30360, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said At2g30360, or a functional equivalent or a homolog thereof as depicted in column 8 of Table II, application no. 19, preferably a homolog or functional equivalent as depicted in column 8 of Table II B, application no. 19, and being depicted in the same respective line as said At2g30360, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “CBL-interacting protein kinase”, preferably it is the molecule of section (a) or (b) of this paragraph.

In particular, it was observed that in plants, especially in Arabidopsis thaliana, increasing or generating the activity of a gene product non-targeted with the activity of a “CBL-interacting protein kinase”, preferably being encoded by a gene comprising the nucleic acid sequence SEQ ID NO. 19919, preferably the coding region thereof, conferred the production of or the increase in bis-glyceryl phosphate (polar fraction) compared with the wild type control.

The nucleic acid sequence of At2g46500 from Arabidopsis thaliana, e.g. as shown in the respective line in column 5 of Table I, application no. 19, has been published in F. And the activity of the gene product thereof is the activity of phosphatidylinositol 3- and 4-kinase family protein.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “phosphatidylinositol 3- and 4-kinase family protein”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said At2g46500, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said At2g46500, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said At2g46500, or a functional equivalent or a homolog thereof as depicted in column 8 of Table II, application no. 19, preferably a homolog or functional equivalent as depicted in column 8 of Table II B, application no. 19, and being depicted in the same respective line as said At2g46500, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “phosphatidylinositol 3- and 4-kinase family protein”, preferably it is the molecule of section (a) or (b) of this paragraph.

In particular, it was observed that in plants, especially in Arabidopsis thaliana, increasing or generating the activity of a gene product non-targeted with the activity of a “phosphatidylinositol 3- and 4-kinase family protein”, preferably being encoded by a gene comprising the nucleic acid sequence SEQ ID NO. 21106, preferably the coding region thereof, conferred the production of or the increase in bis-glyceryl phosphate (polar fraction) compared with the wild type control.

The nucleic acid sequence of At3g02990 from Arabidopsis thaliana, e.g. as shown in the respective line in column 5 of Table I, application no. 19, has been published in F. And the activity of the gene product thereof is the activity of heat shock transcription factor.

Accordingly, in one embodiment, the process of the present invention for producing triacylglycerol in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical triacylglycerol), application no. 19, preferably the coding region thereof, or a homolog or a fragment thereof, and being depicted in the same respective line as said At3g02990, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said At3g02990, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said At3g02990, or a functional equivalent or a homolog thereof as depicted in column 8 of Table II, application no. 19, preferably a homolog or functional equivalent as depicted in column 8 of Table II B, application no. 19, and being depicted in the same respective line as said At3g02990, and preferably the activity is increased non-targeted, whereby the respective line disclose in column 7 the fine chemical triacylglycerol.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 68657, preferably the coding region thereof, conferred the production of or the increase in triacylglycerol compared with the wild type control.

The nucleic acid sequence of At4g09960 from Arabidopsis thaliana, e.g. as shown in the respective line in column 5 of Table I, application no. 19, has been published in F. And the activity of the gene product thereof is the activity of transcription factor.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said At4g09960, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said At4g09960, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said At4g09960, or a functional equivalent or a homolog thereof as depicted in column 8 of Table II, application no. 19, preferably a homolog or functional equivalent as depicted in column 8 of Table II B, application no. 19, and being depicted in the same respective line as said At4g09960, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 109717, preferably the coding region thereof, conferred the production of or the increase in glycerol (lipid fraction) compared with the wild type control.

The nucleic acid sequence of At4g15660 from Arabidopsis thaliana, e.g. as shown in the respective line in column 5 of Table I, application no. 19, has been published in F. And the activity of the gene product thereof is the activity of monothiol glutaredoxin.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said At4g15660, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said At4g15660, or a functional equivalent or a homolog thereof as depicted in column 8 of Table II, application no. 19, preferably a homolog or functional equivalent as depicted in column 8 of Table II B, application no. 19, and being depicted in the same respective line as said At4g15660, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in glycerol (lipid fraction) compared with the wild type control.

The nucleic acid sequence of At4g15660 from Arabidopsis thaliana, e.g. as shown in the respective line in column 5 of Table I, application no. 19, has been published in F. And the activity of the gene product thereof is the activity of monothiol glutaredoxin.

Accordingly, in one embodiment, the process of the present invention for producing triacylglycerol in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical triacylglycerol), application no. 19, preferably the coding region thereof, or a homolog or a fragment thereof, and being depicted in the same respective line as said At4g15660, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said At4g15660, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said At4g15660, or a functional equivalent or a homolog thereof as depicted in column 8 of Table II, application no. 19, preferably a homolog or functional equivalent as depicted in column 8 of Table II B, application no. 19, and being depicted in the same respective line as said At4g15660, and preferably the activity is increased non-targeted, whereby the respective line disclose in column 7 the fine chemical triacylglycerol.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 23482, preferably the coding region thereof, conferred the production of or the increase in triacylglycerol compared with the wild type control.

The nucleic acid sequence of At4g15670 from Arabidopsis thaliana, e.g. as shown in the respective line in column 5 of Table I, application no. 19, has been published in F. And the activity of the gene product thereof is the activity of monothiol glutaredoxin.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said At4g15670, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said At4g15670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said At4g15670, or a functional equivalent or a homolog thereof as depicted in column 8 of Table II, application no. 19, preferably a homolog or functional equivalent as depicted in column 8 of Table II B, application no. 19, and being depicted in the same respective line as said At4g15670, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3279, preferably the coding region thereof, conferred the production of or the increase in glycerol (lipid fraction) compared with the wild type control.

The nucleic acid sequence of At4g15670 from Arabidopsis thaliana, e.g. as shown in the respective line in column 5 of Table I, application no. 19, has been published in F. And the activity of the gene product thereof is the activity of monothiol glutaredoxin.

Accordingly, in one embodiment, the process of the present invention for producing triacylglycerol in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical triacylglycerol), application no. 19, preferably the coding region thereof, or a homolog or a fragment thereof, and being depicted in the same respective line as said At4g15670, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said At4g15670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said At4g15670, or a functional equivalent or a homolog thereof as depicted in column 8 of Table II, application no. 19, preferably a homolog or functional equivalent as depicted in column 8 of Table II B, application no. 19, and being depicted in the same respective line as said At4g15670, and preferably the activity is increased non-targeted, whereby the respective line disclose in column 7 the fine chemical triacylglycerol.

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 3279, preferably the coding region thereof, conferred the production of or the increase in triacylglycerol compared with the wild type control.

The nucleic acid sequence of At4g26080 from Arabidopsis thaliana, e.g. as shown in the respective line in column 5 of Table I, application no. 19, has been published in F. And the activity of the gene product thereof is the activity of protein phosphatase.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said At4g26080, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said At4g26080, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said At4g26080, or a functional equivalent or a homolog thereof as depicted in column 8 of Table II, application no. 19, preferably a homolog or functional equivalent as depicted in column 8 of Table II B, application no. 19, and being depicted in the same respective line as said At4g26080, and preferably the activity is increased non-targeted, whereby the respective line disclose in column 7 the fine chemical glycerol phosphate (lipid fraction).

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 68849, preferably the coding region thereof, conferred the production of or the increase in glycerol phosphate (lipid fraction) compared with the wild type control.

The nucleic acid sequence of At4g34160 from Arabidopsis thaliana, e.g. as shown in the respective line in column 5 of Table I, application no. 19, has been published in F. And the activity of the gene product thereof is the activity of cyclin D .

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “cyclin D”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said At4g34160, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said At4g34160, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said At4g34160, or a functional equivalent or a homolog thereof as depicted in column 8 of Table II, application no. 19, preferably a homolog or functional equivalent as depicted in column 8 of Table II B, application no. 19, and being depicted in the same respective line as said At4g34160, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 24311, preferably the coding region thereof, conferred the production of or the increase in bis-glyceryl phosphate (polar fraction) compared with the wild type control.

The nucleic acid sequence of At4g35310 from Arabidopsis thaliana, e.g. as shown in the respective line in column 5 of Table I, application no. 19, has been published in F. And the activity of the gene product thereof is the activity of calcium-dependent protein kinase.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “calcium-dependent protein kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said At4g35310, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said At4g35310, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said At4g35310, or a functional equivalent or a homolog thereof as depicted in column 8 of Table II, application no. 19, preferably a homolog or functional equivalent as depicted in column 8 of Table II B, application no. 19, and being depicted in the same respective line as said At4g35310, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “calcium-dependent protein kinase”, preferably it is the molecule of section (a) or (b) of this paragraph.

In particular, it was observed that in plants, especially in Arabidopsis thaliana, increasing or generating the activity of a gene product non-targeted with the activity of a “calcium-dependent protein kinase”, preferably being encoded by a gene comprising the nucleic acid sequence SEQ ID NO. 4348, preferably the coding region thereof, conferred the production of or the increase in bis-glyceryl phosphate (polar fraction) compared with the wild type control.

The nucleic acid sequence of At5g03720 from Arabidopsis thaliana, e.g. as shown in the respective line in column 5 of Table I, application no. 19, has been published in F. And the activity of the gene product thereof is the activity of heat shock transcription factor.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said At5g03720, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said At5g03720, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said At5g03720, or a functional equivalent or a homolog thereof as depicted in column 8 of Table II, application no. 19, preferably a homolog or functional equivalent as depicted in column 8 of Table II B, application no. 19, and being depicted in the same respective line as said At5g03720, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 24438, preferably the coding region thereof, conferred the production of or the increase in glycerol (lipid fraction) compared with the wild type control.

The nucleic acid sequence of At5g03720 from Arabidopsis thaliana, e.g. as shown in the respective line in column 5 of Table I, application no. 19, has been published in F. And the activity of the gene product thereof is the activity of heat shock transcription factor.

Accordingly, in one embodiment, the process of the present invention for producing triacylglycerol in a non-human organism, like a microorganism or a plant or a part thereof, comprises increasing or generating the activity of a gene product with the activity of a gene product conferring the activity of “heat shock transcription factor”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical triacylglycerol), application no. 19, preferably the coding region thereof, or a homolog or a fragment thereof, and being depicted in the same respective line as said At5g03720, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said At5g03720, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said At5g03720, or a functional equivalent or a homolog thereof as depicted in column 8 of Table II, application no. 19, preferably a homolog or functional equivalent as depicted in column 8 of Table II B, application no. 19, and being depicted in the same respective line as said At5g03720, and preferably the activity is increased non-targeted, whereby the respective line disclose in column 7 the fine chemical triacylglycerol.

Accordingly, in one embodiment, the molecule which activity is to be increased in the process of the invention is the gene product with an activity as a “heat shock transcription factor”, preferably it is the molecule of section (a) or (b) of this paragraph.

In particular, it was observed that in plants, especially in Arabidopsis thaliana, increasing or generating the activity of a gene product non-targeted with the activity of a “heat shock transcription factor”, preferably being encoded by a gene comprising the nucleic acid sequence SEQ ID NO. 24438, preferably the coding region thereof, conferred the production of or the increase in triacylglycerol compared with the wild type control.

The nucleic acid sequence of At5g18600 from Arabidopsis thaliana, e.g. as shown in the respective line in column 5 of Table I, application no. 19, has been published in F. And the activity of the gene product thereof is the activity of monothiol glutaredoxin.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “monothiol glutaredoxin”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said At5g18600, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said At5g18600, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said At5g18600, or a functional equivalent or a homolog thereof as depicted in column 8 of Table II, application no. 19, preferably a homolog or functional equivalent as depicted in column 8 of Table II B, application no. 19, and being depicted in the same respective line as said At5g18600, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “monothiol glutaredoxin”, preferably it is the molecule of section (a) or (b) of this paragraph.

In particular, it was observed that in plants, especially in Arabidopsis thaliana, increasing or generating the activity of a gene product non-targeted with the activity of a “monothiol glutaredoxin”, preferably being encoded by a gene comprising the nucleic acid sequence SEQ ID NO. 4904, preferably the coding region thereof, conferred the production of or the increase in glycerol (lipid fraction) compared with the wild type control.

The nucleic acid sequence of At5g57050 from Arabidopsis thaliana, e.g. as shown in the respective line in column 5 of Table I, application no. 19, has been published in F. And the activity of the gene product thereof is the activity of protein phosphatase.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said At5g57050, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said At5g57050, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said At5g57050, or a functional equivalent or a homolog thereof as depicted in column 8 of Table II, application no. 19, preferably a homolog or functional equivalent as depicted in column 8 of Table II B, application no. 19, and being depicted in the same respective line as said At5g57050, and preferably the activity is increased non-targeted, whereby the respective line disclose in column 7 the fine chemical glycerol phosphate (lipid fraction).

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 5318, preferably the coding region thereof, conferred the production of or the increase in glycerol phosphate (lipid fraction) compared with the wild type control.

The nucleic acid sequence of At5g59220 from Arabidopsis thaliana, e.g. as shown in the respective line in column 5 of Table I, application no. 19, has been published in F. And the activity of the gene product thereof is the activity of protein phosphatase.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “protein phosphatase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said At5g59220, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said At5g59220, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said At5g59220, or a functional equivalent or a homolog thereof as depicted in column 8 of Table II, application no. 19, preferably a homolog or functional equivalent as depicted in column 8 of Table II B, application no. 19, and being depicted in the same respective line as said At5g59220, and preferably the activity is increased non-targeted, whereby the respective line disclose in column 7 the fine chemical glycerol phosphate (lipid fraction).

Accordingly, in one embodiment, the molecule which activity is to be increased in the process of the invention is the gene product with an activity as a “protein phosphatase”, preferably it is the molecule of section (a) or (b) of this paragraph.

In particular, it was observed that in plants, especially in Arabidopsis thaliana, increasing or generating the activity of a gene product non-targeted with the activity of a “protein phosphatase”, preferably being encoded by a gene comprising the nucleic acid sequence SEQ ID NO. 25498, preferably the coding region thereof, conferred the production of or the increase in glycerol phosphate (lipid fraction) compared with the wild type control.

The nucleic acid sequence of At5g63680 from Arabidopsis thaliana, e.g. as shown in the respective line in column 5 of Table I, application no. 19, has been published in F. And the activity of the gene product thereof is the activity of pyruvate kinase.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “pyruvate kinase”, especially from Arabidopsis thaliana or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said At5g63680, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said At5g63680, and preferably the activity is increased plastidic, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said At5g63680, or a functional equivalent or a homolog thereof as depicted in column 8 of Table II, application no. 19, preferably a homolog or functional equivalent as depicted in column 8 of Table II B, application no. 19, and being depicted in the same respective line as said At5g63680, and preferably the activity is increased plastidic, whereby the respective line disclose in column 7 the fine chemical glycerol phosphate (lipid fraction).

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 70038, preferably the coding region thereof, conferred the production of or the increase in glycerol phosphate (lipid fraction) compared with the wild type control.

The nucleic acid sequence of AVINDRAFT_{—}0539 from Azotobacter vinelandii, e.g. as shown in the respective line in column 5 of Table I, application no. 19, has been published in D. And the activity of the gene product thereof is the activity of Glycine cleavage system T aminomethyltransferase.

Accordingly, in one embodiment, the process of the present invention for producing triacylglycerol in a non-human organism, like a microorganism or a plant or a part thereof, comprises increasing or generating the activity of a gene product with the activity of a gene product conferring the activity of “Glycine cleavage system T aminomethyltransferase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical triacylglycerol), application no. 19, preferably the coding region thereof, or a homolog or a fragment thereof, and being depicted in the same respective line as said AVINDRAFT_{—}0539, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said AVINDRAFT_{—}0539, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said AVINDRAFT_{—}0539, or a functional equivalent or a homolog thereof as depicted in column 8 of Table II, application no. 19, preferably a homolog or functional equivalent as depicted in column 8 of Table II B, application no. 19, and being depicted in the same respective line as said AVINDRAFT_{—}0539, and preferably the activity is increased non-targeted, whereby the respective line disclose in column 7 the fine chemical triacylglycerol.

Accordingly, in one embodiment, the molecule which activity is to be increased in the process of the invention is the gene product with an activity as a “Glycine cleavage system T aminomethyltransferase”, preferably it is the molecule of section (a) or (b) of this paragraph.

In particular, it was observed that in plants, especially in Arabidopsis thaliana, increasing or generating the activity of a gene product non-targeted with the activity of a “Glycine cleavage system T aminomethyltransferase”, preferably being encoded by a gene comprising the nucleic acid sequence SEQ ID NO. 70715, preferably the coding region thereof, conferred the production of or the increase in triacylglycerol compared with the wild type control.

The nucleic acid sequence of AvinDRAFT_{—}1624 from Azotobacter vinelandii, e.g. as shown in the respective line in column 5 of Table I, application no. 19, has been published in D. And the activity of the gene product thereof is the activity of sec-independent protein translocase.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “sec-independent protein translocase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said AvinDRAFT_{—}1624, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said AvinDRAFT_{—}1624, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said AvinDRAFT_{—}1624, or a functional equivalent or a homolog thereof as depicted in column 8 of Table II, application no. 19, preferably a homolog or functional equivalent as depicted in column 8 of Table II B, application no. 19, and being depicted in the same respective line as said AvinDRAFT_{—}1624, and preferably the activity is increased non-targeted, whereby the respective line disclose in column 7 the fine chemical glycerol phosphate (lipid fraction).

Accordingly, in one embodiment, the molecule which activity is to be increased in the process of the invention is the gene product with an activity as a “sec-independent protein translocase”, preferably it is the molecule of section (a) or (b) of this paragraph.

In particular, it was observed that in plants, especially in Arabidopsis thaliana, increasing or generating the activity of a gene product non-targeted with the activity of a “sec-independent protein translocase”, preferably being encoded by a gene comprising the nucleic acid sequence SEQ ID NO. 26196, preferably the coding region thereof, conferred the production of or the increase in glycerol phosphate (lipid fraction) compared with the wild type control.

The nucleic acid sequence of AvinDRAFT_{—}1788 from Azotobacter vinelandii, e.g. as shown in the respective line in column 5 of Table I, application no. 19, has been published in D. And the activity of the gene product thereof is the activity of oxidoreductase.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “oxidoreductase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said AvinDRAFT_{—}1788, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said AvinDRAFT_{—}1788, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said AvinDRAFT_{—}1788, or a functional equivalent or a homolog thereof as depicted in column 8 of Table II, application no. 19, preferably a homolog or functional equivalent as depicted in column 8 of Table II B, application no. 19, and being depicted in the same respective line as said AvinDRAFT_{—}1788, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 114083, preferably the coding region thereof, conferred the production of or the increase in glycerol (lipid fraction) compared with the wild type control.

The nucleic acid sequence of AvinDRAFT_{—}2010 from Azotobacter vinelandii, e.g. as shown in the respective line in column 5 of Table I, application no. 19, has been published in D. And the activity of the gene product thereof is the activity of transcriptional regulator protein.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “transcriptional regulator protein”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said AvinDRAFT_{—}2010, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said AvinDRAFT_{—}2010, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said AvinDRAFT_{—}2010, or a functional equivalent or a homolog thereof as depicted in column 8 of Table II, application no. 19, preferably a homolog or functional equivalent as depicted in column 8 of Table II B, application no. 19, and being depicted in the same respective line as said AvinDRAFT_{—}2010, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “transcriptional regulator protein”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator protein”, preferably being encoded by a gene comprising the nucleic acid sequence SEQ ID NO. 114198, preferably the coding region thereof, conferred the production of or the increase in bis-glyceryl phosphate (polar fraction) compared with the wild type control.

The nucleic acid sequence of AvinDRAFT_{—}2091 from Azotobacter vinelandii, e.g. as shown in the respective line in column 5 of Table I, application no. 19, has been published in D. And the activity of the gene product thereof is the activity of beta-hydroxylase.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “beta-hydroxylase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said AvinDRAFT_{—}2091, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said AvinDRAFT_{—}2091, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said AvinDRAFT_{—}2091, or a functional equivalent or a homolog thereof as depicted in column 8 of Table II, application no. 19, preferably a homolog or functional equivalent as depicted in column 8 of Table II B, application no. 19, and being depicted in the same respective line as said AvinDRAFT_{—}2091, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “beta-hydroxylase”, preferably it is the molecule of section (a) or (b) of this paragraph.

In particular, it was observed that in plants, especially in Arabidopsis thaliana, increasing or generating the activity of a gene product non-targeted with the activity of a “beta-hydroxylase”, preferably being encoded by a gene comprising the nucleic acid sequence SEQ ID NO. 6040, preferably the coding region thereof, conferred the production of or the increase in bis-glyceryl phosphate (polar fraction) compared with the wild type control.

The nucleic acid sequence of AvinDRAFT_{—}2091 from Azotobacter vinelandii, e.g. as shown in the respective line in column 5 of Table I, application no. 19, has been published in D. And the activity of the gene product thereof is the activity of beta-hydroxylase.

Accordingly, in one embodiment, the process of the present invention for producing triacylglycerol in a non-human organism, like a microorganism or a plant or a part thereof, comprises increasing or generating the activity of a gene product with the activity of a gene product conferring the activity of “beta-hydroxylase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical triacylglycerol), application no. 19, preferably the coding region thereof, or a homolog or a fragment thereof, and being depicted in the same respective line as said AvinDRAFT_{—}2091, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said AvinDRAFT_{—}2091, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said AvinDRAFT_{—}2091, or a functional equivalent or a homolog thereof as depicted in column 8 of Table II, application no. 19, preferably a homolog or functional equivalent as depicted in column 8 of Table II B, application no. 19, and being depicted in the same respective line as said AvinDRAFT_{—}2091, and preferably the activity is increased non-targeted, whereby the respective line disclose in column 7 the fine chemical triacylglycerol.

Accordingly, in one embodiment, the molecule which activity is to be increased in the process of the invention is the gene product with an activity as a “beta-hydroxylase”, preferably it is the molecule of section (a) or (b) of this paragraph.

In particular, it was observed that in plants, especially in Arabidopsis thaliana, increasing or generating the activity of a gene product non-targeted with the activity of a “beta-hydroxylase”, preferably being encoded by a gene comprising the nucleic acid sequence SEQ ID NO. 6040, preferably the coding region thereof, conferred the production of or the increase in triacylglycerol compared with the wild type control.

The nucleic acid sequence of AvinDRAFT_{—}3253 from Azotobacter vinelandii, e.g. as shown in the respective line in column 5 of Table I, application no. 19, has been published in D. And the activity of the gene product thereof is the activity of oxidoreductase.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “oxidoreductase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said AvinDRAFT_{—}3253, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said AvinDRAFT_{—}3253, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said AvinDRAFT_{—}3253, or a functional equivalent or a homolog thereof as depicted in column 8 of Table II, application no. 19, preferably a homolog or functional equivalent as depicted in column 8 of Table II B, application no. 19, and being depicted in the same respective line as said AvinDRAFT_{—}3253, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 29500, preferably the coding region thereof, conferred the production of or the increase in bis-glyceryl phosphate (polar fraction) compared with the wild type control.

The nucleic acid sequence of AvinDRAFT_{—}3577 from Azotobacter vinelandii, e.g. as shown in the respective line in column 5 of Table I, application no. 19, has been published in D. And the activity of the gene product thereof is the activity of ThiF family protein.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “ThiF family protein”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said AvinDRAFT_{—}3577, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said AvinDRAFT_{—}3577, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said AvinDRAFT_{—}3577, or a functional equivalent or a homolog thereof as depicted in column 8 of Table II, application no. 19, preferably a homolog or functional equivalent as depicted in column 8 of Table II B, application no. 19, and being depicted in the same respective line as said AvinDRAFT_{—}3577, and preferably the activity is increased non-targeted, whereby the respective line disclose in column 7 the fine chemical glycerol phosphate (lipid fraction).

Accordingly, in one embodiment, the molecule which activity is to be increased in the process of the invention is the gene product with an activity as a “ThiF family protein”, preferably it is the molecule of section (a) or (b) of this paragraph.

In particular, it was observed that in plants, especially in Arabidopsis thaliana, increasing or generating the activity of a gene product non-targeted with the activity of a “ThiF family protein”, preferably being encoded by a gene comprising the nucleic acid sequence SEQ ID NO. 111155, preferably the coding region thereof, conferred the production of or the increase in glycerol phosphate (lipid fraction) compared with the wild type control.

The nucleic acid sequence of AvinDRAFT_{—}3629 from Azotobacter vinelandii, e.g. as shown in the respective line in column 5 of Table I, application no. 19, has been published in D. And the activity of the gene product thereof is the activity of recombinase A.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “recombinase A”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said AvinDRAFT_{—}3629, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said AvinDRAFT_{—}3629, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said AvinDRAFT_{—}3629, or a functional equivalent or a homolog thereof as depicted in column 8 of Table II, application no. 19, preferably a homolog or functional equivalent as depicted in column 8 of Table II B, application no. 19, and being depicted in the same respective line as said AvinDRAFT_{—}3629, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “recombinase A”, preferably it is the molecule of section (a) or (b) of this paragraph.

In particular, it was observed that in plants, especially in Arabidopsis thaliana, increasing or generating the activity of a gene product non-targeted with the activity of a “recombinase A”, preferably being encoded by a gene comprising the nucleic acid sequence SEQ ID NO. 114231, preferably the coding region thereof, conferred the production of or the increase in glycerol (lipid fraction) compared with the wild type control.

The nucleic acid sequence of AvinDRAFT_{—}4606 from Azotobacter vinelandii, e.g. as shown in the respective line in column 5 of Table I, application no. 19, has been published in D. And the activity of the gene product thereof is the activity of NADH-quinone oxidoreductase subunit.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “NADH-quinone oxidoreductase subunit”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said AvinDRAFT_{—}4606, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said AvinDRAFT_{—}4606, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said AvinDRAFT_{—}4606, or a functional equivalent or a homolog thereof as depicted in column 8 of Table II, application no. 19, preferably a homolog or functional equivalent as depicted in column 8 of Table II B, application no. 19, and being depicted in the same respective line as said AvinDRAFT_{—}4606, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “NADH-quinone oxidoreductase subunit”, preferably it is the molecule of section (a) or (b) of this paragraph.

In particular, it was observed that in plants, especially in Arabidopsis thaliana, increasing or generating the activity of a gene product non-targeted with the activity of a “NADH-quinone oxidoreductase subunit”, preferably being encoded by a gene comprising the nucleic acid sequence SEQ ID NO. 73719, preferably the coding region thereof, conferred the production of or the increase in bis-glyceryl phosphate (polar fraction) compared with the wild type control.

The nucleic acid sequence of AvinDRAFT_{—}5103 from Azotobacter vinelandii, e.g. as shown in the respective line in column 5 of Table I, application no. 19, has been published in D. And the activity of the gene product thereof is the activity of hydrolase.

Accordingly, in one embodiment, the process of the present invention for producing triacylglycerol in a non-human organism, like a microorganism or a plant or a part thereof, comprises increasing or generating the activity of a gene product with the activity of a gene product conferring the activity of “hydrolase”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical triacylglycerol), application no. 19, preferably the coding region thereof, or a homolog or a fragment thereof, and being depicted in the same respective line as said AvinDRAFT_{—}5103, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said AvinDRAFT_{—}5103, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said AvinDRAFT_{—}5103, or a functional equivalent or a homolog thereof as depicted in column 8 of Table II, application no. 19, preferably a homolog or functional equivalent as depicted in column 8 of Table II B, application no. 19, and being depicted in the same respective line as said AvinDRAFT_{—}5103, and preferably the activity is increased non-targeted, whereby the respective line disclose in column 7 the fine chemical triacylglycerol.

Accordingly, in one embodiment, the molecule which activity is to be increased in the process of the invention is the gene product with an activity as a “hydrolase”, preferably it is the molecule of section (a) or (b) of this paragraph.

In particular, it was observed that in plants, especially in Arabidopsis thaliana, increasing or generating the activity of a gene product non-targeted with the activity of a “hydrolase”, preferably being encoded by a gene comprising the nucleic acid sequence SEQ ID NO. 6510, preferably the coding region thereof, conferred the production of or the increase in triacylglycerol compared with the wild type control.

The nucleic acid sequence of AvinDRAFT_{—}5467 from Azotobacter vinelandii, e.g. as shown in the respective line in column 5 of Table I, application no. 19, has been published in D. And the activity of the gene product thereof is the activity of HesB/YadR/YfhF family protein.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “HesB/YadR/YfhF family protein”, especially from Azotobacter vinelandii or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said AvinDRAFT_{—}5467, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said AvinDRAFT_{—}5467, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said AvinDRAFT_{—}5467, or a functional equivalent or a homolog thereof as depicted in column 8 of Table II, application no. 19, preferably a homolog or functional equivalent as depicted in column 8 of Table II B, application no. 19, and being depicted in the same respective line as said AvinDRAFT_{—}5467, and preferably the activity is increased non-targeted, whereby the respective line disclose in column 7 the fine chemical glycerol phosphate (lipid fraction).

Accordingly, in one embodiment, the molecule which activity is to be increased in the process of the invention is the gene product with an activity as a “HesB/YadR/YfhF family protein”, preferably it is the molecule of section (a) or (b) of this paragraph.

In particular, it was observed that in plants, especially in Arabidopsis thaliana, increasing or generating the activity of a gene product non-targeted with the activity of a “HesB/YadR/YfhF family protein”, preferably being encoded by a gene comprising the nucleic acid sequence SEQ ID NO. 32648, preferably the coding region thereof, conferred the production of or the increase in glycerol phosphate (lipid fraction) compared with the wild type control.

The nucleic acid sequence of AY623894 from Zea mays, e.g. as shown in the respective line in column 5 of Table I, application no. 19, is unpublished. And the activity of the gene product thereof is the activity of pyruvate kinase.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “pyruvate kinase”, especially from Zea mays or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said AY623894, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said AY623894, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said AY623894, or a functional equivalent or a homolog thereof as depicted in column 8 of Table II, application no. 19, preferably a homolog or functional equivalent as depicted in column 8 of Table II B, application no. 19, and being depicted in the same respective line as said AY623894, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “pyruvate kinase”, preferably it is the molecule of section (a) or (b) of this paragraph.

In particular, it was observed that in plants, especially in Arabidopsis thaliana, increasing or generating the activity of a gene product non-targeted with the activity of a “pyruvate kinase”, preferably being encoded by a gene comprising the nucleic acid sequence SEQ ID NO. 103433, preferably the coding region thereof, conferred the production of or the increase in bis-glyceryl phosphate (polar fraction) compared with the wild type control.

The nucleic acid sequence of B0004 from Escherichia coli, e.g. as shown in the respective line in column 5 of Table I, application no. 19, has been published in B. And the activity of the gene product thereof is the activity of threonine synthase.

Accordingly, in one embodiment, the process of the present invention for producing triacylglycerol in a non-human organism, like a microorganism or a plant or a part thereof, comprises increasing or generating the activity of a gene product with the activity of a gene product conferring the activity of “threonine synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical triacylglycerol), application no. 19, preferably the coding region thereof, or a homolog or a fragment thereof, and being depicted in the same respective line as said B0004, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B0004, and preferably the activity is increased plastidic, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B0004, or a functional equivalent or a homolog thereof as depicted in column 8 of Table II, application no. 19, preferably a homolog or functional equivalent as depicted in column 8 of Table II B, application no. 19, and being depicted in the same respective line as said B0004, and preferably the activity is increased plastidic, whereby the respective line disclose in column 7 the fine chemical triacylglycerol.

Accordingly, in one embodiment, the molecule which activity is to be increased in the process of the invention is the gene product with an activity as a “threonine synthase”, preferably it is the molecule of section (a) or (b) of this paragraph.

In particular, it was observed that in plants, especially in Arabidopsis thaliana, increasing or generating the activity of a gene product plastidic with the activity of a “threonine synthase”, preferably being encoded by a gene comprising the nucleic acid sequence SEQ ID NO. 34889, preferably the coding region thereof, conferred the production of or the increase in triacylglycerol compared with the wild type control.

The nucleic acid sequence of B0050 from Escherichia coli, e.g. as shown in the respective line in column 5 of Table I, application no. 19, has been published in B. And the activity of the gene product thereof is the activity of b0050-protein.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “b0050-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said B0050, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B0050, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B0050, or a functional equivalent or a homolog thereof as depicted in column 8 of Table II, application no. 19, preferably a homolog or functional equivalent as depicted in column 8 of Table II B, application no. 19, and being depicted in the same respective line as said B0050, and preferably the activity is increased non-targeted, whereby the respective line disclose in column 7 the fine chemical glycerol phosphate (lipid fraction).

Accordingly, in one embodiment, the molecule which activity is to be increased in the process of the invention is the gene product with an activity as a “b0050-protein”, preferably it is the molecule of section (a) or (b) of this paragraph.

In particular, it was observed that in plants, especially in Arabidopsis thaliana, increasing or generating the activity of a gene product non-targeted with the activity of a “b0050-protein”, preferably being encoded by a gene comprising the nucleic acid sequence SEQ ID NO. 103958, preferably the coding region thereof, conferred the production of or the increase in glycerol phosphate (lipid fraction) compared with the wild type control.

The nucleic acid sequence of B0161 from Escherichia coli, e.g. as shown in the respective line in column 5 of Table I, application no. 19, has been published in B. And the activity of the gene product thereof is the activity of serine protease.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “serine protease”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B0161, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B0161, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B0161, or a functional equivalent or a homolog thereof as depicted in column 8 of Table II, application no. 19, preferably a homolog or functional equivalent as depicted in column 8 of Table II B, application no. 19, and being depicted in the same respective line as said B0161, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “serine protease”, preferably it is the molecule of section (a) or (b) of this paragraph.

In particular, it was observed that in plants, especially in Arabidopsis thaliana, increasing or generating the activity of a gene product non-targeted with the activity of a “serine protease”, preferably being encoded by a gene comprising the nucleic acid sequence SEQ ID NO. 7081, preferably the coding region thereof, conferred the production of or the increase in bis-glyceryl phosphate (polar fraction) compared with the wild type control.

The nucleic acid sequence of B0161 from Escherichia coli, e.g. as shown in the respective line in column 5 of Table I, application no. 19, has been published in B. And the activity of the gene product thereof is the activity of serine protease.

Accordingly, in one embodiment, the process of the present invention for producing triacylglycerol in a non-human organism, like a microorganism or a plant or a part thereof, comprises increasing or generating the activity of a gene product with the activity of a gene product conferring the activity of “serine protease”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical triacylglycerol), application no. 19, preferably the coding region thereof, or a homolog or a fragment thereof, and being depicted in the same respective line as said B0161, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B0161, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B0161, or a functional equivalent or a homolog thereof as depicted in column 8 of Table II, application no. 19, preferably a homolog or functional equivalent as depicted in column 8 of Table II B, application no. 19, and being depicted in the same respective line as said B0161, and preferably the activity is increased non-targeted, whereby the respective line disclose in column 7 the fine chemical triacylglycerol.

Accordingly, in one embodiment, the molecule which activity is to be increased in the process of the invention is the gene product with an activity as a “serine protease”, preferably it is the molecule of section (a) or (b) of this paragraph.

In particular, it was observed that in plants, especially in Arabidopsis thaliana, increasing or generating the activity of a gene product non-targeted with the activity of a “serine protease”, preferably being encoded by a gene comprising the nucleic acid sequence SEQ ID NO. 7081, preferably the coding region thereof, conferred the production of or the increase in triacylglycerol compared with the wild type control.

The nucleic acid sequence of B0449 from Escherichia coli, e.g. as shown in the respective line in column 5 of Table I, application no. 19, has been published in B. And the activity of the gene product thereof is the activity of ATP-binding component of a transport system.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “ATP-binding component of a transport system”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B0449, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B0449, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B0449, or a functional equivalent or a homolog thereof as depicted in column 8 of Table II, application no. 19, preferably a homolog or functional equivalent as depicted in column 8 of Table II B, application no. 19, and being depicted in the same respective line as said B0449, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “ATP-binding component of a transport system”, preferably it is the molecule of section (a) or (b) of this paragraph.

In particular, it was observed that in plants, especially in Arabidopsis thaliana, increasing or generating the activity of a gene product non-targeted with the activity of a “ATP-binding component of a transport system”, preferably being encoded by a gene comprising the nucleic acid sequence SEQ ID NO. 7333, preferably the coding region thereof, conferred the production of or the increase in bis-glyceryl phosphate (polar fraction) compared with the wild type control.

The nucleic acid sequence of B0518 from Escherichia coli, e.g. as shown in the respective line in column 5 of Table I, application no. 19, has been published in B. And the activity of the gene product thereof is the activity of b0518-protein.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “b0518-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said B0518, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B0518, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B0518, or a functional equivalent or a homolog thereof as depicted in column 8 of Table II, application no. 19, preferably a homolog or functional equivalent as depicted in column 8 of Table II B, application no. 19, and being depicted in the same respective line as said B0518, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “b0518-protein”, preferably it is the molecule of section (a) or (b) of this paragraph.

In particular, it was observed that in plants, especially in Arabidopsis thaliana, increasing or generating the activity of a gene product non-targeted with the activity of a “b0518-protein”, preferably being encoded by a gene comprising the nucleic acid sequence SEQ ID NO. 35936, preferably the coding region thereof, conferred the production of or the increase in glycerol (lipid fraction) compared with the wild type control.

The nucleic acid sequence of B0755 from Escherichia coli, e.g. as shown in the respective line in column 5 of Table I, application no. 19, has been published in B. And the activity of the gene product thereof is the activity of phosphoglyceromutase.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “phosphoglyceromutase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B0755, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B0755, and preferably the activity is increased plastidic, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B0755, or a functional equivalent or a homolog thereof as depicted in column 8 of Table II, application no. 19, preferably a homolog or functional equivalent as depicted in column 8 of Table II B, application no. 19, and being depicted in the same respective line as said B0755, and preferably the activity is increased plastidic, whereby the respective line disclose in column 7 the fine chemical bis-glyceryl phosphate (polar fraction).

Accordingly, in one embodiment, the molecule which activity is to be increased in the process of the invention is the gene product with an activity as a “phosphoglyceromutase”, preferably it is the molecule of section (a) or (b) of this paragraph.

In particular, it was observed that in plants, especially in Arabidopsis thaliana, increasing or generating the activity of a gene product plastidic with the activity of a “phosphoglyceromutase”, preferably being encoded by a gene comprising the nucleic acid sequence SEQ ID NO. 152576, preferably the coding region thereof, conferred the production of or the increase in bis-glyceryl phosphate (polar fraction) compared with the wild type control.

The nucleic acid sequence of B0856 from Escherichia coli, e.g. as shown in the respective line in column 5 of Table I, application no. 19, has been published in B. And the activity of the gene product thereof is the activity of putrescine transport system permease protein.

Accordingly, in one embodiment, the process of the present invention for producing glycerol (polar fraction) in a non-human organism, like a microorganism or a plant or a part thereof, comprises increasing or generating the activity of a gene product with the activity of a gene product conferring the activity of “putrescine transport system permease protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (polar fraction)), application no. 19, preferably the coding region thereof, or a homolog or a fragment thereof, and being depicted in the same respective line as said B0856, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B0856, and preferably the activity is increased plastidic, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B0856, or a functional equivalent or a homolog thereof as depicted in column 8 of Table II, application no. 19, preferably a homolog or functional equivalent as depicted in column 8 of Table II B, application no. 19, and being depicted in the same respective line as said B0856, and preferably the activity is increased plastidic, whereby the respective line disclose in column 7 the fine chemical glycerol (polar fraction).

Accordingly, in one embodiment, the molecule which activity is to be increased in the process of the invention is the gene product with an activity as a “putrescine transport system permease protein”, preferably it is the molecule of section (a) or (b) of this paragraph.

In particular, it was observed that in plants, especially in Arabidopsis thaliana, increasing or generating the activity of a gene product plastidic with the activity of a “putrescine transport system permease protein”, preferably being encoded by a gene comprising the nucleic acid sequence SEQ ID NO. 131359, preferably the coding region thereof, conferred the production of or the increase in glycerol (polar fraction) compared with the wild type control.

The nucleic acid sequence of B0963 from Escherichia coli, e.g. as shown in the respective line in column 5 of Table I, application no. 19, has been published in B. And the activity of the gene product thereof is the activity of methylglyoxal synthase.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “methylglyoxal synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said B0963, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B0963, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B0963, or a functional equivalent or a homolog thereof as depicted in column 8 of Table II, application no. 19, preferably a homolog or functional equivalent as depicted in column 8 of Table II B, application no. 19, and being depicted in the same respective line as said B0963, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “methylglyoxal synthase”, preferably it is the molecule of section (a) or (b) of this paragraph.

In particular, it was observed that in plants, especially in Arabidopsis thaliana, increasing or generating the activity of a gene product non-targeted with the activity of a “methylglyoxal synthase”, preferably being encoded by a gene comprising the nucleic acid sequence SEQ ID NO. 36670, preferably the coding region thereof, conferred the production of or the increase in glycerol (lipid fraction) compared with the wild type control.

The nucleic acid sequence of B0963 from Escherichia coli, e.g. as shown in the respective line in column 5 of Table I, application no. 19, has been published in B. And the activity of the gene product thereof is the activity of methylglyoxal synthase.

Accordingly, in one embodiment, the process of the present invention for producing glycerol (polar fraction) in a non-human organism, like a microorganism or a plant or a part thereof, comprises increasing or generating the activity of a gene product with the activity of a gene product conferring the activity of “methylglyoxal synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (polar fraction)), application no. 19, preferably the coding region thereof, or a homolog or a fragment thereof, and being depicted in the same respective line as said B0963, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B0963, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B0963, or a functional equivalent or a homolog thereof as depicted in column 8 of Table II, application no. 19, preferably a homolog or functional equivalent as depicted in column 8 of Table II B, application no. 19, and being depicted in the same respective line as said B0963, and preferably the activity is increased non-targeted, whereby the respective line disclose in column 7 the fine chemical glycerol (polar fraction).

Accordingly, in one embodiment, the molecule which activity is to be increased in the process of the invention is the gene product with an activity as a “methylglyoxal synthase”, preferably it is the molecule of section (a) or (b) of this paragraph.

In particular, it was observed that in plants, especially in Arabidopsis thaliana, increasing or generating the activity of a gene product non-targeted with the activity of a “methylglyoxal synthase”, preferably being encoded by a gene comprising the nucleic acid sequence SEQ ID NO. 36670, preferably the coding region thereof, conferred the production of or the increase in glycerol (polar fraction) compared with the wild type control.

The nucleic acid sequence of B0970 from Escherichia coli, e.g. as shown in the respective line in column 5 of Table I, application no. 19, has been published in B. And the activity of the gene product thereof is the activity of b0970-protein.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “b0970-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B0970, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B0970, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B0970, or a functional equivalent or a homolog thereof as depicted in column 8 of Table II, application no. 19, preferably a homolog or functional equivalent as depicted in column 8 of Table II B, application no. 19, and being depicted in the same respective line as said B0970, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “b0970-protein”, preferably it is the molecule of section (a) or (b) of this paragraph.

In particular, it was observed that in plants, especially in Arabidopsis thaliana, increasing or generating the activity of a gene product non-targeted with the activity of a “b0970-protein”, preferably being encoded by a gene comprising the nucleic acid sequence SEQ ID NO. 144479, preferably the coding region thereof, conferred the production of or the increase in bis-glyceryl phosphate (polar fraction) compared with the wild type control.

The nucleic acid sequence of B1003 from Escherichia coli, e.g. as shown in the respective line in column 5 of Table I, application no. 19, has been published in B. And the activity of the gene product thereof is the activity of b1003-protein.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “b1003-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B1003, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B1003, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B1003, or a functional equivalent or a homolog thereof as depicted in column 8 of Table II, application no. 19, preferably a homolog or functional equivalent as depicted in column 8 of Table II B, application no. 19, and being depicted in the same respective line as said B1003, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “b1003-protein”, preferably it is the molecule of section (a) or (b) of this paragraph.

In particular, it was observed that in plants, especially in Arabidopsis thaliana, increasing or generating the activity of a gene product non-targeted with the activity of a “b1003-protein”, preferably being encoded by a gene comprising the nucleic acid sequence SEQ ID NO. 7941, preferably the coding region thereof, conferred the production of or the increase in bis-glyceryl phosphate (polar fraction) compared with the wild type control.

The nucleic acid sequence of B1095 from Escherichia coli, e.g. as shown in the respective line in column 5 of Table I, application no. 19, has been published in B. And the activity of the gene product thereof is the activity of 3-oxoacyl-[acyl-carrier-protein] synthase.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “3-oxoacyl-[acyl-carrier-protein] synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B1095, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B1095, and preferably the activity is increased plastidic, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B1095, or a functional equivalent or a homolog thereof as depicted in column 8 of Table II, application no. 19, preferably a homolog or functional equivalent as depicted in column 8 of Table II B, application no. 19, and being depicted in the same respective line as said B1095, and preferably the activity is increased plastidic, whereby the respective line disclose in column 7 the fine chemical bis-glyceryl phosphate (polar fraction).

Accordingly, in one embodiment, the molecule which activity is to be increased in the process of the invention is the gene product with an activity as a “3-oxoacyl-[acyl-carrier-protein] synthase”, preferably it is the molecule of section (a) or (b) of this paragraph.

In particular, it was observed that in plants, especially in Arabidopsis thaliana, increasing or generating the activity of a gene product plastidic with the activity of a “3-oxoacyl-[acyl-carrier-protein] synthase”, preferably being encoded by a gene comprising the nucleic acid sequence SEQ ID NO. 114552, preferably the coding region thereof, conferred the production of or the increase in bis-glyceryl phosphate (polar fraction) compared with the wild type control.

The nucleic acid sequence of B1186 from Escherichia coli, e.g. as shown in the respective line in column 5 of Table I, application no. 19, has been published in B. And the activity of the gene product thereof is the activity of sodium/proton antiporter.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “sodium/proton antiporter”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said B1186, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B1186, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B1186, or a functional equivalent or a homolog thereof as depicted in column 8 of Table II, application no. 19, preferably a homolog or functional equivalent as depicted in column 8 of Table II B, application no. 19, and being depicted in the same respective line as said B1186, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “sodium/proton antiporter”, preferably it is the molecule of section (a) or (b) of this paragraph.

In particular, it was observed that in plants, especially in Arabidopsis thaliana, increasing or generating the activity of a gene product non-targeted with the activity of a “sodium/proton antiporter”, preferably being encoded by a gene comprising the nucleic acid sequence SEQ ID NO. 37400, preferably the coding region thereof, conferred the production of or the increase in glycerol (lipid fraction) compared with the wild type control.

The nucleic acid sequence of B1234 from Escherichia coli, e.g. as shown in the respective line in column 5 of Table I, application no. 19, has been published in B. And the activity of the gene product thereof is the activity of b1234-protein.

Accordingly, in one embodiment, the process of the present invention for producing triacylglycerol in a non-human organism, like a microorganism or a plant or a part thereof, comprises increasing or generating the activity of a gene product with the activity of a gene product conferring the activity of “b1234-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical triacylglycerol), application no. 19, preferably the coding region thereof, or a homolog or a fragment thereof, and being depicted in the same respective line as said B1234, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B1234, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B1234, or a functional equivalent or a homolog thereof as depicted in column 8 of Table II, application no. 19, preferably a homolog or functional equivalent as depicted in column 8 of Table II B, application no. 19, and being depicted in the same respective line as said B1234, and preferably the activity is increased non-targeted, whereby the respective line disclose in column 7 the fine chemical triacylglycerol.

Accordingly, in one embodiment, the molecule which activity is to be increased in the process of the invention is the gene product with an activity as a “b1234-protein”, preferably it is the molecule of section (a) or (b) of this paragraph.

In particular, it was observed that in plants, especially in Arabidopsis thaliana, increasing or generating the activity of a gene product non-targeted with the activity of a “b1234-protein”, preferably being encoded by a gene comprising the nucleic acid sequence SEQ ID NO. 112111, preferably the coding region thereof, conferred the production of or the increase in triacylglycerol compared with the wild type control.

The nucleic acid sequence of B1249 from Escherichia coli, e.g. as shown in the respective line in column 5 of Table I, application no. 19, has been published in B. And the activity of the gene product thereof is the activity of cardiolipin synthase.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “cardiolipin synthase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said B1249, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B1249, and preferably the activity is increased plastidic, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B1249, or a functional equivalent or a homolog thereof as depicted in column 8 of Table II, application no. 19, preferably a homolog or functional equivalent as depicted in column 8 of Table II B, application no. 19, and being depicted in the same respective line as said B1249, and preferably the activity is increased plastidic, whereby the respective line disclose in column 7 the fine chemical glycerol (lipid fraction).

Accordingly, in one embodiment, the molecule which activity is to be increased in the process of the invention is the gene product with an activity as a “cardiolipin synthase”, preferably it is the molecule of section (a) or (b) of this paragraph.

In particular, it was observed that in plants, especially in Arabidopsis thaliana, increasing or generating the activity of a gene product plastidic with the activity of a “cardiolipin synthase”, preferably being encoded by a gene comprising the nucleic acid sequence SEQ ID NO. 98778, preferably the coding region thereof, conferred the production of or the increase in glycerol (lipid fraction) compared with the wild type control.

The nucleic acid sequence of B1330 from Escherichia coli, e.g. as shown in the respective line in column 5 of Table I, application no. 19, has been published in B. And the activity of the gene product thereof is the activity of b1330-protein.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “b1330-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said B1330, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B1330, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B1330, or a functional equivalent or a homolog thereof as depicted in column 8 of Table II, application no. 19, preferably a homolog or functional equivalent as depicted in column 8 of Table II B, application no. 19, and being depicted in the same respective line as said B1330, and preferably the activity is increased non-targeted, whereby the respective line disclose in column 7 the fine chemical glycerol phosphate (lipid fraction).

Accordingly, in one embodiment, the molecule which activity is to be increased in the process of the invention is the gene product with an activity as a “b1330-protein”, preferably it is the molecule of section (a) or (b) of this paragraph.

In particular, it was observed that in plants, especially in Arabidopsis thaliana, increasing or generating the activity of a gene product non-targeted with the activity of a “b1330-protein”, preferably being encoded by a gene comprising the nucleic acid sequence SEQ ID NO. 38226, preferably the coding region thereof, conferred the production of or the increase in glycerol phosphate (lipid fraction) compared with the wild type control.

The nucleic acid sequence of B1343 from Escherichia coli, e.g. as shown in the respective line in column 5 of Table I, application no. 19, has been published in B. And the activity of the gene product thereof is the activity of ATP-dependent RNA helicase.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “ATP-dependent RNA helicase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B1343, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B1343, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B1343, or a functional equivalent or a homolog thereof as depicted in column 8 of Table II, application no. 19, preferably a homolog or functional equivalent as depicted in column 8 of Table II B, application no. 19, and being depicted in the same respective line as said B1343, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “ATP-dependent RNA helicase”, preferably it is the molecule of section (a) or (b) of this paragraph.

In particular, it was observed that in plants, especially in Arabidopsis thaliana, increasing or generating the activity of a gene product non-targeted with the activity of a “ATP-dependent RNA helicase”, preferably being encoded by a gene comprising the nucleic acid sequence SEQ ID NO. 76890, preferably the coding region thereof, conferred the production of or the increase in bis-glyceryl phosphate (polar fraction) compared with the wild type control.

The nucleic acid sequence of B1391 from Escherichia coli, e.g. as shown in the respective line in column 5 of Table I, application no. 19, has been published in B. And the activity of the gene product thereof is the activity of phenylacetic acid degradation protein.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “phenylacetic acid degradation protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B1391, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B1391, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B1391, or a functional equivalent or a homolog thereof as depicted in column 8 of Table II, application no. 19, preferably a homolog or functional equivalent as depicted in column 8 of Table II B, application no. 19, and being depicted in the same respective line as said B1391, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “phenylacetic acid degradation protein”, preferably it is the molecule of section (a) or (b) of this paragraph.

In particular, it was observed that in plants, especially in Arabidopsis thaliana, increasing or generating the activity of a gene product non-targeted with the activity of a “phenylacetic acid degradation protein”, preferably being encoded by a gene comprising the nucleic acid sequence SEQ ID NO. 131489, preferably the coding region thereof, conferred the production of or the increase in bis-glyceryl phosphate (polar fraction) compared with the wild type control.

The nucleic acid sequence of B1431 from Escherichia coli, e.g. as shown in the respective line in column 5 of Table I, application no. 19, has been published in B. And the activity of the gene product thereof is the activity of lipoprotein precursor.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “lipoprotein precursor”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B1431, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B1431, and preferably the activity is increased plastidic, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B1431, or a functional equivalent or a homolog thereof as depicted in column 8 of Table II, application no. 19, preferably a homolog or functional equivalent as depicted in column 8 of Table II B, application no. 19, and being depicted in the same respective line as said B1431, and preferably the activity is increased plastidic, whereby the respective line disclose in column 7 the fine chemical bis-glyceryl phosphate (polar fraction).

Accordingly, in one embodiment, the molecule which activity is to be increased in the process of the invention is the gene product with an activity as a “lipoprotein precursor”, preferably it is the molecule of section (a) or (b) of this paragraph.

In particular, it was observed that in plants, especially in Arabidopsis thaliana, increasing or generating the activity of a gene product plastidic with the activity of a “lipoprotein precursor”, preferably being encoded by a gene comprising the nucleic acid sequence SEQ ID NO. 38266, preferably the coding region thereof, conferred the production of or the increase in bis-glyceryl phosphate (polar fraction) compared with the wild type control.

The nucleic acid sequence of B1522 from Escherichia coli, e.g. as shown in the respective line in column 5 of Table I, application no. 19, has been published in B. And the activity of the gene product thereof is the activity of b1522-protein.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “b1522-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B1522, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B1522, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B1522, or a functional equivalent or a homolog thereof as depicted in column 8 of Table II, application no. 19, preferably a homolog or functional equivalent as depicted in column 8 of Table II B, application no. 19, and being depicted in the same respective line as said B1522, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “b1522-protein”, preferably it is the molecule of section (a) or (b) of this paragraph.

In particular, it was observed that in plants, especially in Arabidopsis thaliana, increasing or generating the activity of a gene product non-targeted with the activity of a “b1522-protein”, preferably being encoded by a gene comprising the nucleic acid sequence SEQ ID NO. 7947, preferably the coding region thereof, conferred the production of or the increase in bis-glyceryl phosphate (polar fraction) compared with the wild type control.

The nucleic acid sequence of B1554 from Escherichia coli, e.g. as shown in the respective line in column 5 of Table I, application no. 19, has been published in B. And the activity of the gene product thereof is the activity of lysozyme.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “lysozyme”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B1554, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B1554, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B1554, or a functional equivalent or a homolog thereof as depicted in column 8 of Table II, application no. 19, preferably a homolog or functional equivalent as depicted in column 8 of Table II B, application no. 19, and being depicted in the same respective line as said B1554, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “lysozyme”, preferably it is the molecule of section (a) or (b) of this paragraph.

In particular, it was observed that in plants, especially in Arabidopsis thaliana, increasing or generating the activity of a gene product non-targeted with the activity of a “lysozyme”, preferably being encoded by a gene comprising the nucleic acid sequence SEQ ID NO. 152991, preferably the coding region thereof, conferred the production of or the increase in bis-glyceryl phosphate (polar fraction) compared with the wild type control.

The nucleic acid sequence of B1556 from Escherichia coli, e.g. as shown in the respective line in column 5 of Table I, application no. 19, has been published in B. And the activity of the gene product thereof is the activity of b1556-protein.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “b1556-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B1556, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B1556, and preferably the activity is increased plastidic, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B1556, or a functional equivalent or a homolog thereof as depicted in column 8 of Table II, application no. 19, preferably a homolog or functional equivalent as depicted in column 8 of Table II B, application no. 19, and being depicted in the same respective line as said B1556, and preferably the activity is increased plastidic, whereby the respective line disclose in column 7 the fine chemical bis-glyceryl phosphate (polar fraction).

Accordingly, in one embodiment, the molecule which activity is to be increased in the process of the invention is the gene product with an activity as a “b1556-protein”, preferably it is the molecule of section (a) or (b) of this paragraph.

In particular, it was observed that in plants, especially in Arabidopsis thaliana, increasing or generating the activity of a gene product plastidic with the activity of a “b1556-protein”, preferably being encoded by a gene comprising the nucleic acid sequence SEQ ID NO. 145142, preferably the coding region thereof, conferred the production of or the increase in bis-glyceryl phosphate (polar fraction) compared with the wild type control.

The nucleic acid sequence of B1583 from Escherichia coli, e.g. as shown in the respective line in column 5 of Table I, application no. 19, has been published in B. And the activity of the gene product thereof is the activity of b1583-protein.

Accordingly, in one embodiment, the process of the present invention for producing triacylglycerol in a non-human organism, like a microorganism or a plant or a part thereof, comprises increasing or generating the activity of a gene product with the activity of a gene product conferring the activity of “b1583-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical triacylglycerol), application no. 19, preferably the coding region thereof, or a homolog or a fragment thereof, and being depicted in the same respective line as said B1583, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B1583, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B1583, or a functional equivalent or a homolog thereof as depicted in column 8 of Table II, application no. 19, preferably a homolog or functional equivalent as depicted in column 8 of Table II B, application no. 19, and being depicted in the same respective line as said B1583, and preferably the activity is increased non-targeted, whereby the respective line disclose in column 7 the fine chemical triacylglycerol.

Accordingly, in one embodiment, the molecule which activity is to be increased in the process of the invention is the gene product with an activity as a “b1583-protein”, preferably it is the molecule of section (a) or (b) of this paragraph.

In particular, it was observed that in plants, especially in Arabidopsis thaliana, increasing or generating the activity of a gene product non-targeted with the activity of a “b1583-protein”, preferably being encoded by a gene comprising the nucleic acid sequence SEQ ID NO. 78138, preferably the coding region thereof, conferred the production of or the increase in triacylglycerol compared with the wild type control.

The nucleic acid sequence of B1589 from Escherichia coli, e.g. as shown in the respective line in column 5 of Table I, application no. 19, has been published in B. And the activity of the gene product thereof is the activity of Fe—S subunit of oxidoreductase.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “Fe—S subunit of oxidoreductase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B1589, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B1589, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B1589, or a functional equivalent or a homolog thereof as depicted in column 8 of Table II, application no. 19, preferably a homolog or functional equivalent as depicted in column 8 of Table II B, application no. 19, and being depicted in the same respective line as said B1589, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “Fe—S subunit of oxidoreductase”, preferably it is the molecule of section (a) or (b) of this paragraph.

In particular, it was observed that in plants, especially in Arabidopsis thaliana, increasing or generating the activity of a gene product non-targeted with the activity of a “Fe—S subunit of oxidoreductase”, preferably being encoded by a gene comprising the nucleic acid sequence SEQ ID NO. 78154, preferably the coding region thereof, conferred the production of or the increase in bis-glyceryl phosphate (polar fraction) compared with the wild type control.

The nucleic acid sequence of B1597 from Escherichia coli, e.g. as shown in the respective line in column 5 of Table I, application no. 19, has been published in B. And the activity of the gene product thereof is the activity of acid shock protein.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “acid shock protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said B1597, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B1597, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B1597, or a functional equivalent or a homolog thereof as depicted in column 8 of Table II, application no. 19, preferably a homolog or functional equivalent as depicted in column 8 of Table II B, application no. 19, and being depicted in the same respective line as said B1597, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “acid shock protein”, preferably it is the molecule of section (a) or (b) of this paragraph.

In particular, it was observed that in plants, especially in Arabidopsis thaliana, increasing or generating the activity of a gene product non-targeted with the activity of a “acid shock protein”, preferably being encoded by a gene comprising the nucleic acid sequence SEQ ID NO. 38300, preferably the coding region thereof, conferred the production of or the increase in glycerol (lipid fraction) compared with the wild type control.

The nucleic acid sequence of B1670 from Escherichia coli, e.g. as shown in the respective line in column 5 of Table I, application no. 19, has been published in B. And the activity of the gene product thereof is the activity of b1670-protein.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “b1670-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B1670, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B1670, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B1670, or a functional equivalent or a homolog thereof as depicted in column 8 of Table II, application no. 19, preferably a homolog or functional equivalent as depicted in column 8 of Table II B, application no. 19, and being depicted in the same respective line as said B1670, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “b1670-protein”, preferably it is the molecule of section (a) or (b) of this paragraph.

In particular, it was observed that in plants, especially in Arabidopsis thaliana, increasing or generating the activity of a gene product non-targeted with the activity of a “b1670-protein”, preferably being encoded by a gene comprising the nucleic acid sequence SEQ ID NO. 78753, preferably the coding region thereof, conferred the production of or the increase in bis-glyceryl phosphate (polar fraction) compared with the wild type control.

The nucleic acid sequence of B1672 from Escherichia coli, e.g. as shown in the respective line in column 5 of Table I, application no. 19, has been published in B. And the activity of the gene product thereof is the activity of b1672-protein.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “b1672-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B1672, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B1672, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B1672, or a functional equivalent or a homolog thereof as depicted in column 8 of Table II, application no. 19, preferably a homolog or functional equivalent as depicted in column 8 of Table II B, application no. 19, and being depicted in the same respective line as said B1672, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “b1672-protein”, preferably it is the molecule of section (a) or (b) of this paragraph.

In particular, it was observed that in plants, especially in Arabidopsis thaliana, increasing or generating the activity of a gene product non-targeted with the activity of a “b1672-protein”, preferably being encoded by a gene comprising the nucleic acid sequence SEQ ID NO. 78771, preferably the coding region thereof, conferred the production of or the increase in bis-glyceryl phosphate (polar fraction) compared with the wild type control.

The nucleic acid sequence of B1703 from Escherichia coli, e.g. as shown in the respective line in column 5 of Table I, application no. 19, has been published in B. And the activity of the gene product thereof is the activity of b1703-protein.

Accordingly, in one embodiment, the process of the present invention for producing triacylglycerol in a non-human organism, like a microorganism or a plant or a part thereof, comprises increasing or generating the activity of a gene product with the activity of a gene product conferring the activity of “b1703-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical triacylglycerol), application no. 19, preferably the coding region thereof, or a homolog or a fragment thereof, and being depicted in the same respective line as said B1703, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B1703, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B1703, or a functional equivalent or a homolog thereof as depicted in column 8 of Table II, application no. 19, preferably a homolog or functional equivalent as depicted in column 8 of Table II B, application no. 19, and being depicted in the same respective line as said B1703, and preferably the activity is increased non-targeted, whereby the respective line disclose in column 7 the fine chemical triacylglycerol.

Accordingly, in one embodiment, the molecule which activity is to be increased in the process of the invention is the gene product with an activity as a “b1703-protein”, preferably it is the molecule of section (a) or (b) of this paragraph.

In particular, it was observed that in plants, especially in Arabidopsis thaliana, increasing or generating the activity of a gene product non-targeted with the activity of a “b1703-protein”, preferably being encoded by a gene comprising the nucleic acid sequence SEQ ID NO. 124907, preferably the coding region thereof, conferred the production of or the increase in triacylglycerol compared with the wild type control.

The nucleic acid sequence of B1736 from Escherichia coli, e.g. as shown in the respective line in column 5 of Table I, application no. 19, has been published in B. And the activity of the gene product thereof is the activity of phosphotransferase system component.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “phosphotransferase system component”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B1736, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B1736, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B1736, or a functional equivalent or a homolog thereof as depicted in column 8 of Table II, application no. 19, preferably a homolog or functional equivalent as depicted in column 8 of Table II B, application no. 19, and being depicted in the same respective line as said B1736, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “phosphotransferase system component”, preferably it is the molecule of section (a) or (b) of this paragraph.

In particular, it was observed that in plants, especially in Arabidopsis thaliana, increasing or generating the activity of a gene product non-targeted with the activity of a “phosphotransferase system component”, preferably being encoded by a gene comprising the nucleic acid sequence SEQ ID NO. 153072, preferably the coding region thereof, conferred the production of or the increase in bis-glyceryl phosphate (polar fraction) compared with the wild type control.

The nucleic acid sequence of B1795 from Escherichia coli, e.g. as shown in the respective line in column 5 of Table I, application no. 19, has been published in B. And the activity of the gene product thereof is the activity of B1795-protein.

Accordingly, in one embodiment, the process of the present invention for producing triacylglycerol in a non-human organism, like a microorganism or a plant or a part thereof, comprises increasing or generating the activity of a gene product with the activity of a gene product conferring the activity of “B1795-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical triacylglycerol), application no. 19, preferably the coding region thereof, or a homolog or a fragment thereof, and being depicted in the same respective line as said B1795, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B1795, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B1795, or a functional equivalent or a homolog thereof as depicted in column 8 of Table II, application no. 19, preferably a homolog or functional equivalent as depicted in column 8 of Table II B, application no. 19, and being depicted in the same respective line as said B1795, and preferably the activity is increased non-targeted, whereby the respective line disclose in column 7 the fine chemical triacylglycerol.

Accordingly, in one embodiment, the molecule which activity is to be increased in the process of the invention is the gene product with an activity as a “B1795-protein”, preferably it is the molecule of section (a) or (b) of this paragraph.

In particular, it was observed that in plants, especially in Arabidopsis thaliana, increasing or generating the activity of a gene product non-targeted with the activity of a “B1795-protein”, preferably being encoded by a gene comprising the nucleic acid sequence SEQ ID NO. 131539, preferably the coding region thereof, conferred the production of or the increase in triacylglycerol compared with the wild type control.

The nucleic acid sequence of B1814 from Escherichia coli, e.g. as shown in the respective line in column 5 of Table I, application no. 19, has been published in B. And the activity of the gene product thereof is the activity of L-serine dehydratase.

Accordingly, in one embodiment, the process of the present invention for producing glycerol-3-phosphate (polar fraction) in a non-human organism, like a microorganism or a plant or a part thereof, comprises increasing or generating the activity of a gene product with the activity of a gene product conferring the activity of “L-serine dehydratase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol-3-phosphate (polar fraction)), application no. 19, preferably the coding region thereof, or a homolog or a fragment thereof, and being depicted in the same respective line as said B1814, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B1814, and preferably the activity is increased plastidic, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B1814, or a functional equivalent or a homolog thereof as depicted in column 8 of Table II, application no. 19, preferably a homolog or functional equivalent as depicted in column 8 of Table II B, application no. 19, and being depicted in the same respective line as said B1814, and preferably the activity is increased plastidic, whereby the respective line disclose in column 7 the fine chemical glycerol-3-phosphate (polar fraction).

Accordingly, in one embodiment, the molecule which activity is to be increased in the process of the invention is the gene product with an activity as a “L-serine dehydratase”, preferably it is the molecule of section (a) or (b) of this paragraph.

In particular, it was observed that in plants, especially in Arabidopsis thaliana, increasing or generating the activity of a gene product plastidic with the activity of a “L-serine dehydratase”, preferably being encoded by a gene comprising the nucleic acid sequence SEQ ID NO. 8033, preferably the coding region thereof, conferred the production of or the increase in glycerol-3-phosphate (polar fraction) compared with the wild type control.

The nucleic acid sequence of B1837 from Escherichia coli, e.g. as shown in the respective line in column 5 of Table I, application no. 19, has been published in B. And the activity of the gene product thereof is the activity of b1837-protein.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “b1837-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said B1837, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B1837, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B1837, or a functional equivalent or a homolog thereof as depicted in column 8 of Table II, application no. 19, preferably a homolog or functional equivalent as depicted in column 8 of Table II B, application no. 19, and being depicted in the same respective line as said B1837, and preferably the activity is increased non-targeted, whereby the respective line disclose in column 7 the fine chemical glycerol phosphate (lipid fraction).

Accordingly, in one embodiment, the molecule which activity is to be increased in the process of the invention is the gene product with an activity as a “b1837-protein”, preferably it is the molecule of section (a) or (b) of this paragraph.

In particular, it was observed that in plants, especially in Arabidopsis thaliana, increasing or generating the activity of a gene product non-targeted with the activity of a “b1837-protein”, preferably being encoded by a gene comprising the nucleic acid sequence SEQ ID NO. 78953, preferably the coding region thereof, conferred the production of or the increase in glycerol phosphate (lipid fraction) compared with the wild type control.

The nucleic acid sequence of B1897 from Escherichia coli, e.g. as shown in the respective line in column 5 of Table I, application no. 19, has been published in B. And the activity of the gene product thereof is the activity of trehalose-phosphatase.

Accordingly, in one embodiment, the process of the present invention for producing triacylglycerol in a non-human organism, like a microorganism or a plant or a part thereof, comprises increasing or generating the activity of a gene product with the activity of a gene product conferring the activity of “trehalose-phosphatase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical triacylglycerol), application no. 19, preferably the coding region thereof, or a homolog or a fragment thereof, and being depicted in the same respective line as said B1897, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B1897, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B1897, or a functional equivalent or a homolog thereof as depicted in column 8 of Table II, application no. 19, preferably a homolog or functional equivalent as depicted in column 8 of Table II B, application no. 19, and being depicted in the same respective line as said B1897, and preferably the activity is increased non-targeted, whereby the respective line disclose in column 7 the fine chemical triacylglycerol.

Accordingly, in one embodiment, the molecule which activity is to be increased in the process of the invention is the gene product with an activity as a “trehalose-phosphatase”, preferably it is the molecule of section (a) or (b) of this paragraph.

In particular, it was observed that in plants, especially in Arabidopsis thaliana, increasing or generating the activity of a gene product non-targeted with the activity of a “trehalosephosphatase”, preferably being encoded by a gene comprising the nucleic acid sequence SEQ ID NO. 79056, preferably the coding region thereof, conferred the production of or the increase in triacylglycerol compared with the wild type control.

The nucleic acid sequence of B2023 from Escherichia coli, e.g. as shown in the respective line in column 5 of Table I, application no. 19, has been published in B. And the activity of the gene product thereof is the activity of imidazole glycerol phosphate synthase subunit.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “imidazole glycerol phosphate synthase subunit”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said B2023, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B2023, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B2023, or a functional equivalent or a homolog thereof as depicted in column 8 of Table II, application no. 19, preferably a homolog or functional equivalent as depicted in column 8 of Table II B, application no. 19, and being depicted in the same respective line as said B2023, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “imidazole glycerol phosphate synthase subunit”, preferably it is the molecule of section (a) or (b) of this paragraph.

In particular, it was observed that in plants, especially in Arabidopsis thaliana, increasing or generating the activity of a gene product non-targeted with the activity of a “imidazole glycerol phosphate synthase subunit”, preferably being encoded by a gene comprising the nucleic acid sequence SEQ ID NO. 99371, preferably the coding region thereof, conferred the production of or the increase in glycerol (lipid fraction) compared with the wild type control.

The nucleic acid sequence of B2032 from Escherichia coli, e.g. as shown in the respective line in column 5 of Table I, application no. 19, has been published in B. And the activity of the gene product thereof is the activity of b2032-protein.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “b2032-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said B2032, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B2032, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B2032, or a functional equivalent or a homolog thereof as depicted in column 8 of Table II, application no. 19, preferably a homolog or functional equivalent as depicted in column 8 of Table II B, application no. 19, and being depicted in the same respective line as said B2032, and preferably the activity is increased non-targeted, whereby the respective line disclose in column 7 the fine chemical glycerol phosphate (lipid fraction).

Accordingly, in one embodiment, the molecule which activity is to be increased in the process of the invention is the gene product with an activity as a “b2032-protein”, preferably it is the molecule of section (a) or (b) of this paragraph.

In particular, it was observed that in plants, especially in Arabidopsis thaliana, increasing or generating the activity of a gene product non-targeted with the activity of a “b2032-protein”, preferably being encoded by a gene comprising the nucleic acid sequence SEQ ID NO. 8920, preferably the coding region thereof, conferred the production of or the increase in glycerol phosphate (lipid fraction) compared with the wild type control.

The nucleic acid sequence of B2099 from Escherichia coli, e.g. as shown in the respective line in column 5 of Table I, application no. 19, has been published in B. And the activity of the gene product thereof is the activity of b2099-protein.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “b2099-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said B2099, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B2099, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B2099, or a functional equivalent or a homolog thereof as depicted in column 8 of Table II, application no. 19, preferably a homolog or functional equivalent as depicted in column 8 of Table II B, application no. 19, and being depicted in the same respective line as said B2099, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “b2099-protein”, preferably it is the molecule of section (a) or (b) of this paragraph.

In particular, it was observed that in plants, especially in Arabidopsis thaliana, increasing or generating the activity of a gene product non-targeted with the activity of a “b2099-protein”, preferably being encoded by a gene comprising the nucleic acid sequence SEQ ID NO. 115595, preferably the coding region thereof, conferred the production of or the increase in glycerol (lipid fraction) compared with the wild type control.

The nucleic acid sequence of B2139 from Escherichia coli, e.g. as shown in the respective line in column 5 of Table I, application no. 19, has been published in B. And the activity of the gene product thereof is the activity of b2139-protein.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “b2139-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B2139, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B2139, and preferably the activity is increased plastidic, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B2139, or a functional equivalent or a homolog thereof as depicted in column 8 of Table II, application no. 19, preferably a homolog or functional equivalent as depicted in column 8 of Table II B, application no. 19, and being depicted in the same respective line as said B2139, and preferably the activity is increased plastidic, whereby the respective line disclose in column 7 the fine chemical bis-glyceryl phosphate (polar fraction).

Accordingly, in one embodiment, the molecule which activity is to be increased in the process of the invention is the gene product with an activity as a “b2139-protein”, preferably it is the molecule of section (a) or (b) of this paragraph.

In particular, it was observed that in plants, especially in Arabidopsis thaliana, increasing or generating the activity of a gene product plastidic with the activity of a “b2139-protein”, preferably being encoded by a gene comprising the nucleic acid sequence SEQ ID NO. 125085, preferably the coding region thereof, conferred the production of or the increase in bis-glyceryl phosphate (polar fraction) compared with the wild type control.

The nucleic acid sequence of B2178 from Escherichia coli, e.g. as shown in the respective line in column 5 of Table I, application no. 19, has been published in B. And the activity of the gene product thereof is the activity of ABC transporter permease protein.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “ABC transporter permease protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said B2178, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B2178, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B2178, or a functional equivalent or a homolog thereof as depicted in column 8 of Table II, application no. 19, preferably a homolog or functional equivalent as depicted in column 8 of Table II B, application no. 19, and being depicted in the same respective line as said B2178, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “ABC transporter permease protein”, preferably it is the molecule of section (a) or (b) of this paragraph.

In particular, it was observed that in plants, especially in Arabidopsis thaliana, increasing or generating the activity of a gene product non-targeted with the activity of a “ABC transporter permease protein”, preferably being encoded by a gene comprising the nucleic acid sequence SEQ ID NO. 39040, preferably the coding region thereof, conferred the production of or the increase in glycerol (lipid fraction) compared with the wild type control.

The nucleic acid sequence of B2389 from Escherichia coli, e.g. as shown in the respective line in column 5 of Table I, application no. 19, has been published in B. And the activity of the gene product thereof is the activity of ion-transport protein.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “ion-transport protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said B2389, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B2389, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B2389, or a functional equivalent or a homolog thereof as depicted in column 8 of Table II, application no. 19, preferably a homolog or functional equivalent as depicted in column 8 of Table II B, application no. 19, and being depicted in the same respective line as said B2389, and preferably the activity is increased non-targeted, whereby the respective line disclose in column 7 the fine chemical glycerol phosphate (lipid fraction).

Accordingly, in one embodiment, the molecule which activity is to be increased in the process of the invention is the gene product with an activity as a “ion-transport protein”, preferably it is the molecule of section (a) or (b) of this paragraph.

In particular, it was observed that in plants, especially in Arabidopsis thaliana, increasing or generating the activity of a gene product non-targeted with the activity of a “ion-transport protein”, preferably being encoded by a gene comprising the nucleic acid sequence SEQ ID NO. 146238, preferably the coding region thereof, conferred the production of or the increase in glycerol phosphate (lipid fraction) compared with the wild type control.

The nucleic acid sequence of B2399 from Escherichia coli, e.g. as shown in the respective line in column 5 of Table I, application no. 19, has been published in B. And the activity of the gene product thereof is the activity of b2399-protein.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “b2399-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B2399, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B2399, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B2399, or a functional equivalent or a homolog thereof as depicted in column 8 of Table II, application no. 19, preferably a homolog or functional equivalent as depicted in column 8 of Table II B, application no. 19, and being depicted in the same respective line as said B2399, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “b2399-protein”, preferably it is the molecule of section (a) or (b) of this paragraph.

In particular, it was observed that in plants, especially in Arabidopsis thaliana, increasing or generating the activity of a gene product non-targeted with the activity of a “b2399-protein”, preferably being encoded by a gene comprising the nucleic acid sequence SEQ ID NO. 39237, preferably the coding region thereof, conferred the production of or the increase in bis-glyceryl phosphate (polar fraction) compared with the wild type control.

The nucleic acid sequence of B2399 from Escherichia coli, e.g. as shown in the respective line in column 5 of Table I, application no. 19, has been published in B. And the activity of the gene product thereof is the activity of b2399-protein.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “b2399-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said B2399, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B2399, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B2399, or a functional equivalent or a homolog thereof as depicted in column 8 of Table II, application no. 19, preferably a homolog or functional equivalent as depicted in column 8 of Table II B, application no. 19, and being depicted in the same respective line as said B2399, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “b2399-protein”, preferably it is the molecule of section (a) or (b) of this paragraph.

In particular, it was observed that in plants, especially in Arabidopsis thaliana, increasing or generating the activity of a gene product non-targeted with the activity of a “b2399-protein”, preferably being encoded by a gene comprising the nucleic acid sequence SEQ ID NO. 39237, preferably the coding region thereof, conferred the production of or the increase in glycerol (lipid fraction) compared with the wild type control.

The nucleic acid sequence of B2439 from Escherichia coli, e.g. as shown in the respective line in column 5 of Table I, application no. 19, has been published in B. And the activity of the gene product thereof is the activity of ethanolamine utilization protein.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “ethanolamine utilization protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said B2439, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B2439, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B2439, or a functional equivalent or a homolog thereof as depicted in column 8 of Table II, application no. 19, preferably a homolog or functional equivalent as depicted in column 8 of Table II B, application no. 19, and being depicted in the same respective line as said B2439, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “ethanolamine utilization protein”, preferably it is the molecule of section (a) or (b) of this paragraph.

In particular, it was observed that in plants, especially in Arabidopsis thaliana, increasing or generating the activity of a gene product non-targeted with the activity of a “ethanolamine utilization protein”, preferably being encoded by a gene comprising the nucleic acid sequence SEQ ID NO. 115628, preferably the coding region thereof, conferred the production of or the increase in glycerol (lipid fraction) compared with the wild type control.

The nucleic acid sequence of B2439 from Escherichia coli, e.g. as shown in the respective line in column 5 of Table I, application no. 19, has been published in B. And the activity of the gene product thereof is the activity of ethanolamine utilization protein.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “ethanolamine utilization protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said B2439, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B2439, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B2439, or a functional equivalent or a homolog thereof as depicted in column 8 of Table II, application no. 19, preferably a homolog or functional equivalent as depicted in column 8 of Table II B, application no. 19, and being depicted in the same respective line as said B2439, and preferably the activity is increased non-targeted, whereby the respective line disclose in column 7 the fine chemical glycerol phosphate (lipid fraction).

Accordingly, in one embodiment, the molecule which activity is to be increased in the process of the invention is the gene product with an activity as a “ethanolamine utilization protein”, preferably it is the molecule of section (a) or (b) of this paragraph.

In particular, it was observed that in plants, especially in Arabidopsis thaliana, increasing or generating the activity of a gene product non-targeted with the activity of a “ethanolamine utilization protein”, preferably being encoded by a gene comprising the nucleic acid sequence SEQ ID NO. 115628, preferably the coding region thereof, conferred the production of or the increase in glycerol phosphate (lipid fraction) compared with the wild type control.

The nucleic acid sequence of B2461 from Escherichia coli, e.g. as shown in the respective line in column 5 of Table I, application no. 19, has been published in B. And the activity of the gene product thereof is the activity of ethanolamine utilization protein.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “ethanolamine utilization protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said B2461, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B2461, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B2461, or a functional equivalent or a homolog thereof as depicted in column 8 of Table II, application no. 19, preferably a homolog or functional equivalent as depicted in column 8 of Table II B, application no. 19, and being depicted in the same respective line as said B2461, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “ethanolamine utilization protein”, preferably it is the molecule of section (a) or (b) of this paragraph.

In particular, it was observed that in plants, especially in Arabidopsis thaliana, increasing or generating the activity of a gene product non-targeted with the activity of a “ethanolamine utilization protein”, preferably being encoded by a gene comprising the nucleic acid sequence SEQ ID NO. 40299, preferably the coding region thereof, conferred the production of or the increase in glycerol (lipid fraction) compared with the wild type control.

The nucleic acid sequence of B2474 from Escherichia coli, e.g. as shown in the respective line in column 5 of Table I, application no. 19, has been published in B. And the activity of the gene product thereof is the activity of b2474-protein.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “b2474-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B2474, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B2474, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B2474, or a functional equivalent or a homolog thereof as depicted in column 8 of Table II, application no. 19, preferably a homolog or functional equivalent as depicted in column 8 of Table II B, application no. 19, and being depicted in the same respective line as said B2474, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “b2474-protein”, preferably it is the molecule of section (a) or (b) of this paragraph.

In particular, it was observed that in plants, especially in Arabidopsis thaliana, increasing or generating the activity of a gene product non-targeted with the activity of a “b2474-protein”, preferably being encoded by a gene comprising the nucleic acid sequence SEQ ID NO. 40329, preferably the coding region thereof, conferred the production of or the increase in bis-glyceryl phosphate (polar fraction) compared with the wild type control.

The nucleic acid sequence of B2474 from Escherichia coli, e.g. as shown in the respective line in column 5 of Table I, application no. 19, has been published in B. And the activity of the gene product thereof is the activity of b2474-protein.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “b2474-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said B2474, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B2474, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B2474, or a functional equivalent or a homolog thereof as depicted in column 8 of Table II, application no. 19, preferably a homolog or functional equivalent as depicted in column 8 of Table II B, application no. 19, and being depicted in the same respective line as said B2474, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “b2474-protein”, preferably it is the molecule of section (a) or (b) of this paragraph.

In particular, it was observed that in plants, especially in Arabidopsis thaliana, increasing or generating the activity of a gene product non-targeted with the activity of a “b2474-protein”, preferably being encoded by a gene comprising the nucleic acid sequence SEQ ID NO. 40329, preferably the coding region thereof, conferred the production of or the increase in glycerol (lipid fraction) compared with the wild type control.

The nucleic acid sequence of B2546 from Escherichia coli, e.g. as shown in the respective line in column 5 of Table I, application no. 19, has been published in B. And the activity of the gene product thereof is the activity of putative transport system permease protein.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “putative transport system permease protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said B2546, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B2546, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B2546, or a functional equivalent or a homolog thereof as depicted in column 8 of Table II, application no. 19, preferably a homolog or functional equivalent as depicted in column 8 of Table II B, application no. 19, and being depicted in the same respective line as said B2546, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “putative transport system permease protein”, preferably it is the molecule of section (a) or (b) of this paragraph.

In particular, it was observed that in plants, especially in Arabidopsis thaliana, increasing or generating the activity of a gene product non-targeted with the activity of a “putative transport system permease protein”, preferably being encoded by a gene comprising the nucleic acid sequence SEQ ID NO. 99898, preferably the coding region thereof, conferred the production of or the increase in glycerol (lipid fraction) compared with the wild type control.

The nucleic acid sequence of B2546 from Escherichia coli, e.g. as shown in the respective line in column 5 of Table I, application no. 19, has been published in B. And the activity of the gene product thereof is the activity of putative transport system permease protein.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “putative transport system permease protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said B2546, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B2546, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B2546, or a functional equivalent or a homolog thereof as depicted in column 8 of Table II, application no. 19, preferably a homolog or functional equivalent as depicted in column 8 of Table II B, application no. 19, and being depicted in the same respective line as said B2546, and preferably the activity is increased non-targeted, whereby the respective line disclose in column 7 the fine chemical glycerol phosphate (lipid fraction).

Accordingly, in one embodiment, the molecule which activity is to be increased in the process of the invention is the gene product with an activity as a “putative transport system permease protein”, preferably it is the molecule of section (a) or (b) of this paragraph.

In particular, it was observed that in plants, especially in Arabidopsis thaliana, increasing or generating the activity of a gene product non-targeted with the activity of a “putative transport system permease protein”, preferably being encoded by a gene comprising the nucleic acid sequence SEQ ID NO. 99898, preferably the coding region thereof, conferred the production of or the increase in glycerol phosphate (lipid fraction) compared with the wild type control.

The nucleic acid sequence of B2613 from Escherichia coli, e.g. as shown in the respective line in column 5 of Table I, application no. 19, has been published in B. And the activity of the gene product thereof is the activity of b2613-protein.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “b2613-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B2613, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B2613, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B2613, or a functional equivalent or a homolog thereof as depicted in column 8 of Table II, application no. 19, preferably a homolog or functional equivalent as depicted in column 8 of Table II B, application no. 19, and being depicted in the same respective line as said B2613, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “b2613-protein”, preferably it is the molecule of section (a) or (b) of this paragraph.

In particular, it was observed that in plants, especially in Arabidopsis thaliana, increasing or generating the activity of a gene product non-targeted with the activity of a “b2613-protein”, preferably being encoded by a gene comprising the nucleic acid sequence SEQ ID NO. 40665, preferably the coding region thereof, conferred the production of or the increase in bis-glyceryl phosphate (polar fraction) compared with the wild type control.

The nucleic acid sequence of B2634 from Escherichia coli, e.g. as shown in the respective line in column 5 of Table I, application no. 19, has been published in B. And the activity of the gene product thereof is the activity of transcriptional regulator.

Accordingly, in one embodiment, the process of the present invention for producing glycerol (polar fraction) in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (polar fraction)), application no. 19, preferably the coding region thereof, or a homolog or a fragment thereof, and being depicted in the same respective line as said B2634, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B2634, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B2634, or a functional equivalent or a homolog thereof as depicted in column 8 of Table II, application no. 19, preferably a homolog or functional equivalent as depicted in column 8 of Table II B, application no. 19, and being depicted in the same respective line as said B2634, and preferably the activity is increased non-targeted, whereby the respective line disclose in column 7 the fine chemical glycerol (polar fraction).

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 40726, preferably the coding region thereof, conferred the production of or the increase in glycerol (polar fraction) compared with the wild type control.

The nucleic acid sequence of B2673 from Escherichia coli, e.g. as shown in the respective line in column 5 of Table I, application no. 19, has been published in B. And the activity of the gene product thereof is the activity of b2673-protein.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “b2673-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said B2673, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B2673, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B2673, or a functional equivalent or a homolog thereof as depicted in column 8 of Table II, application no. 19, preferably a homolog or functional equivalent as depicted in column 8 of Table II B, application no. 19, and being depicted in the same respective line as said B2673, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “b2673-protein”, preferably it is the molecule of section (a) or (b) of this paragraph.

In particular, it was observed that in plants, especially in Arabidopsis thaliana, increasing or generating the activity of a gene product non-targeted with the activity of a “b2673-protein”, preferably being encoded by a gene comprising the nucleic acid sequence SEQ ID NO. 9244, preferably the coding region thereof, conferred the production of or the increase in glycerol (lipid fraction) compared with the wild type control.

The nucleic acid sequence of B2673 from Escherichia coli, e.g. as shown in the respective line in column 5 of Table I, application no. 19, has been published in B. And the activity of the gene product thereof is the activity of b2673-protein.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “b2673-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said B2673, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B2673, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B2673, or a functional equivalent or a homolog thereof as depicted in column 8 of Table II, application no. 19, preferably a homolog or functional equivalent as depicted in column 8 of Table II B, application no. 19, and being depicted in the same respective line as said B2673, and preferably the activity is increased non-targeted, whereby the respective line disclose in column 7 the fine chemical glycerol phosphate (lipid fraction).

Accordingly, in one embodiment, the molecule which activity is to be increased in the process of the invention is the gene product with an activity as a “b2673-protein”, preferably it is the molecule of section (a) or (b) of this paragraph.

In particular, it was observed that in plants, especially in Arabidopsis thaliana, increasing or generating the activity of a gene product non-targeted with the activity of a “b2673-protein”, preferably being encoded by a gene comprising the nucleic acid sequence SEQ ID NO. 9244, preferably the coding region thereof, conferred the production of or the increase in glycerol phosphate (lipid fraction) compared with the wild type control.

The nucleic acid sequence of B2701 from Escherichia coli, e.g. as shown in the respective line in column 5 of Table I, application no. 19, has been published in B. And the activity of the gene product thereof is the activity of murein transglycosylase.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “murein transglycosylase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B2701, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B2701, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B2701, or a functional equivalent or a homolog thereof as depicted in column 8 of Table II, application no. 19, preferably a homolog or functional equivalent as depicted in column 8 of Table II B, application no. 19, and being depicted in the same respective line as said B2701, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “murein transglycosylase”, preferably it is the molecule of section (a) or (b) of this paragraph.

In particular, it was observed that in plants, especially in Arabidopsis thaliana, increasing or generating the activity of a gene product non-targeted with the activity of a “murein transglycosylase”, preferably being encoded by a gene comprising the nucleic acid sequence SEQ ID NO. 80756, preferably the coding region thereof, conferred the production of or the increase in bis-glyceryl phosphate (polar fraction) compared with the wild type control.

The nucleic acid sequence of B2714 from Escherichia coli, e.g. as shown in the respective line in column 5 of Table I, application no. 19, has been published in B. And the activity of the gene product thereof is the activity of transcriptional regulator.

Accordingly, in one embodiment, the process of the present invention for producing triacylglycerol in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical triacylglycerol), application no. 19, preferably the coding region thereof, or a homolog or a fragment thereof, and being depicted in the same respective line as said B2714, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B2714, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B2714, or a functional equivalent or a homolog thereof as depicted in column 8 of Table II, application no. 19, preferably a homolog or functional equivalent as depicted in column 8 of Table II B, application no. 19, and being depicted in the same respective line as said B2714, and preferably the activity is increased non-targeted, whereby the respective line disclose in column 7 the fine chemical triacylglycerol.

Accordingly, in one embodiment, the molecule which activity is to be increased in the process of the invention is the gene product with an activity as a “transcriptional regulator”, preferably it is the molecule of section (a) or (b) of this paragraph.

In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 40741, preferably the coding region thereof, conferred the production of or the increase in triacylglycerol compared with the wild type control.

The nucleic acid sequence of B2747 from Escherichia coli, e.g. as shown in the respective line in column 5 of Table I, application no. 19, has been published in B. And the activity of the gene product thereof is the activity of 2-C-methyl-D-erythritol 4-phosphate cytidylyltransferase.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “2-C-methyl-D-erythritol 4-phosphate cytidylyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said B2747, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B2747, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B2747, or a functional equivalent or a homolog thereof as depicted in column 8 of Table II, application no. 19, preferably a homolog or functional equivalent as depicted in column 8 of Table II B, application no. 19, and being depicted in the same respective line as said B2747, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “2-C-methyl-D-erythritol 4-phosphate cytidylyltransferase”, preferably it is the molecule of section (a) or (b) of this paragraph.

In particular, it was observed that in plants, especially in Arabidopsis thaliana, increasing or generating the activity of a gene product non-targeted with the activity of a “2-C-methyl-D-erythritol 4-phosphate cytidylyltransferase”, preferably being encoded by a gene comprising the nucleic acid sequence SEQ ID NO. 81075, preferably the coding region thereof, conferred the production of or the increase in glycerol (lipid fraction) compared with the wild type control.

The nucleic acid sequence of B2818 from Escherichia coli, e.g. as shown in the respective line in column 5 of Table I, application no. 19, has been published in B. And the activity of the gene product thereof is the activity of amino-acid acetyltransferase.

Accordingly, in one embodiment, the process of the present invention for producing triacylglycerol in a non-human organism, like a microorganism or a plant or a part thereof, comprises increasing or generating the activity of a gene product with the activity of a gene product conferring the activity of “amino-acid acetyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical triacylglycerol), application no. 19, preferably the coding region thereof, or a homolog or a fragment thereof, and being depicted in the same respective line as said B2818, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B2818, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B2818, or a functional equivalent or a homolog thereof as depicted in column 8 of Table II, application no. 19, preferably a homolog or functional equivalent as depicted in column 8 of Table II B, application no. 19, and being depicted in the same respective line as said B2818, and preferably the activity is increased non-targeted, whereby the respective line disclose in column 7 the fine chemical triacylglycerol.

Accordingly, in one embodiment, the molecule which activity is to be increased in the process of the invention is the gene product with an activity as a “amino-acid acetyltransferase”, preferably it is the molecule of section (a) or (b) of this paragraph.

In particular, it was observed that in plants, especially in Arabidopsis thaliana, increasing or generating the activity of a gene product non-targeted with the activity of a “amino-acid acetyltransferase”, preferably being encoded by a gene comprising the nucleic acid sequence SEQ ID NO. 81576, preferably the coding region thereof, conferred the production of or the increase in triacylglycerol compared with the wild type control.

The nucleic acid sequence of B3083 from Escherichia coli, e.g. as shown in the respective line in column 5 of Table I, application no. 19, has been published in B. And the activity of the gene product thereof is the activity of b3083-protein.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “b3083-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B3083, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B3083, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B3083, or a functional equivalent or a homolog thereof as depicted in column 8 of Table II, application no. 19, preferably a homolog or functional equivalent as depicted in column 8 of Table II B, application no. 19, and being depicted in the same respective line as said B3083, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “b3083-protein”, preferably it is the molecule of section (a) or (b) of this paragraph.

In particular, it was observed that in plants, especially in Arabidopsis thaliana, increasing or generating the activity of a gene product non-targeted with the activity of a “b3083-protein”, preferably being encoded by a gene comprising the nucleic acid sequence SEQ ID NO. 82424, preferably the coding region thereof, conferred the production of or the increase in bis-glyceryl phosphate (polar fraction) compared with the wild type control.

The nucleic acid sequence of B3116 from Escherichia coli, e.g. as shown in the respective line in column 5 of Table I, application no. 19, has been published in B. And the activity of the gene product thereof is the activity of threonine/serine transporter.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “threonine/serine transporter”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B3116, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B3116, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B3116, or a functional equivalent or a homolog thereof as depicted in column 8 of Table II, application no. 19, preferably a homolog or functional equivalent as depicted in column 8 of Table II B, application no. 19, and being depicted in the same respective line as said B3116, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “threonine/serine transporter”, preferably it is the molecule of section (a) or (b) of this paragraph.

In particular, it was observed that in plants, especially in Arabidopsis thaliana, increasing or generating the activity of a gene product non-targeted with the activity of a “threonine/serine transporter”, preferably being encoded by a gene comprising the nucleic acid sequence SEQ ID NO. 153246, preferably the coding region thereof, conferred the production of or the increase in bis-glyceryl phosphate (polar fraction) compared with the wild type control.

The nucleic acid sequence of B3160 from Escherichia coli, e.g. as shown in the respective line in column 5 of Table I, application no. 19, has been published in B. And the activity of the gene product thereof is the activity of b3160-protein.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “b3160-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B3160, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B3160, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B3160, or a functional equivalent or a homolog thereof as depicted in column 8 of Table II, application no. 19, preferably a homolog or functional equivalent as depicted in column 8 of Table II B, application no. 19, and being depicted in the same respective line as said B3160, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “b3160-protein”, preferably it is the molecule of section (a) or (b) of this paragraph.

In particular, it was observed that in plants, especially in Arabidopsis thaliana, increasing or generating the activity of a gene product non-targeted with the activity of a “b3160-protein”, preferably being encoded by a gene comprising the nucleic acid sequence SEQ ID NO. 153395, preferably the coding region thereof, conferred the production of or the increase in bis-glyceryl phosphate (polar fraction) compared with the wild type control.

The nucleic acid sequence of B3172 from Escherichia coli, e.g. as shown in the respective line in column 5 of Table I, application no. 19, has been published in B. And the activity of the gene product thereof is the activity of argininosuccinate synthetase.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “argininosuccinate synthetase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B3172, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B3172, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B3172, or a functional equivalent or a homolog thereof as depicted in column 8 of Table II, application no. 19, preferably a homolog or functional equivalent as depicted in column 8 of Table II B, application no. 19, and being depicted in the same respective line as said B3172, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “argininosuccinate synthetase”, preferably it is the molecule of section (a) or (b) of this paragraph.

In particular, it was observed that in plants, especially in Arabidopsis thaliana, increasing or generating the activity of a gene product non-targeted with the activity of a “argininosuccinate synthetase”, preferably being encoded by a gene comprising the nucleic acid sequence SEQ ID NO. 153618, preferably the coding region thereof, conferred the production of or the increase in bis-glyceryl phosphate (polar fraction) compared with the wild type control.

The nucleic acid sequence of B3256 from Escherichia coli, e.g. as shown in the respective line in column 5 of Table I, application no. 19, has been published in B. And the activity of the gene product thereof is the activity of acetyl CoA carboxylase.

Accordingly, in one embodiment, the process of the present invention for producing glycerol-3-phosphate (polar fraction) in a non-human organism, like a microorganism or a plant or a part thereof, comprises increasing or generating the activity of a gene product with the activity of a gene product conferring the activity of “acetyl CoA carboxylase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol-3-phosphate (polar fraction)), application no. 19, preferably the coding region thereof, or a homolog or a fragment thereof, and being depicted in the same respective line as said B3256, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B3256, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B3256, or a functional equivalent or a homolog thereof as depicted in column 8 of Table II, application no. 19, preferably a homolog or functional equivalent as depicted in column 8 of Table II B, application no. 19, and being depicted in the same respective line as said B3256, and preferably the activity is increased non-targeted, whereby the respective line disclose in column 7 the fine chemical glycerol-3-phosphate (polar fraction).

Accordingly, in one embodiment, the molecule which activity is to be increased in the process of the invention is the gene product with an activity as a “acetyl CoA carboxylase”, preferably it is the molecule of section (a) or (b) of this paragraph.

In particular, it was observed that in plants, especially in Arabidopsis thaliana, increasing or generating the activity of a gene product non-targeted with the activity of a “acetyl CoA carboxylase”, preferably being encoded by a gene comprising the nucleic acid sequence SEQ ID NO. 9492, preferably the coding region thereof, conferred the production of or the increase in glycerol-3-phosphate (polar fraction) compared with the wild type control.

The nucleic acid sequence of B3262 from Escherichia coli, e.g. as shown in the respective line in column 5 of Table I, application no. 19, has been published in B. And the activity of the gene product thereof is the activity of methyltransferase.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “methyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B3262, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B3262, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B3262, or a functional equivalent or a homolog thereof as depicted in column 8 of Table II, application no. 19, preferably a homolog or functional equivalent as depicted in column 8 of Table II B, application no. 19, and being depicted in the same respective line as said B3262, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “methyltransferase”, preferably it is the molecule of section (a) or (b) of this paragraph.

In particular, it was observed that in plants, especially in Arabidopsis thaliana, increasing or generating the activity of a gene product non-targeted with the activity of a “methyltransferase”, preferably being encoded by a gene comprising the nucleic acid sequence SEQ ID NO. 42502, preferably the coding region thereof, conferred the production of or the increase in bis-glyceryl phosphate (polar fraction) compared with the wild type control.

The nucleic acid sequence of B3262 from Escherichia coli, e.g. as shown in the respective line in column 5 of Table I, application no. 19, has been published in B. And the activity of the gene product thereof is the activity of methyltransferase.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “methyltransferase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said B3262, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B3262, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B3262, or a functional equivalent or a homolog thereof as depicted in column 8 of Table II, application no. 19, preferably a homolog or functional equivalent as depicted in column 8 of Table II B, application no. 19, and being depicted in the same respective line as said B3262, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “methyltransferase”, preferably it is the molecule of section (a) or (b) of this paragraph.

In particular, it was observed that in plants, especially in Arabidopsis thaliana, increasing or generating the activity of a gene product non-targeted with the activity of a “methyltransferase”, preferably being encoded by a gene comprising the nucleic acid sequence SEQ ID NO. 42502, preferably the coding region thereof, conferred the production of or the increase in glycerol (lipid fraction) compared with the wild type control.

The nucleic acid sequence of B3347 from Escherichia coli, e.g. as shown in the respective line in column 5 of Table I, application no. 19, has been published in B. And the activity of the gene product thereof is the activity of FKBP-type peptidyl-prolyl cis-trans isomerase (rotamase).

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “FKBP-type peptidyl-prolyl cis-trans isomerase (rotamase)”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B3347, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B3347, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B3347, or a functional equivalent or a homolog thereof as depicted in column 8 of Table II, application no. 19, preferably a homolog or functional equivalent as depicted in column 8 of Table II B, application no. 19, and being depicted in the same respective line as said B3347, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “FKBP-type peptidyl-prolyl cis-trans isomerase (rotamase)”, preferably it is the molecule of section (a) or (b) of this paragraph.

In particular, it was observed that in plants, especially in Arabidopsis thaliana, increasing or generating the activity of a gene product non-targeted with the activity of a “FKBP-type peptidylprolyl cis-trans isomerase (rotamase)”, preferably being encoded by a gene comprising the nucleic acid sequence SEQ ID NO. 153813, preferably the coding region thereof, conferred the production of or the increase in bis-glyceryl phosphate (polar fraction) compared with the wild type control.

The nucleic acid sequence of B3457 from Escherichia coli, e.g. as shown in the respective line in column 5 of Table I, application no. 19, has been published in B. And the activity of the gene product thereof is the activity of high-affinity branched-chain amino acid transport protein.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “high-affinity branched-chain amino acid transport protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B3457, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B3457, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B3457, or a functional equivalent or a homolog thereof as depicted in column 8 of Table II, application no. 19, preferably a homolog or functional equivalent as depicted in column 8 of Table II B, application no. 19, and being depicted in the same respective line as said B3457, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “high-affinity branched-chain amino acid transport protein”, preferably it is the molecule of section (a) or (b) of this paragraph.

In particular, it was observed that in plants, especially in Arabidopsis thaliana, increasing or generating the activity of a gene product non-targeted with the activity of a “high-affinity branched-chain amino acid transport protein”, preferably being encoded by a gene comprising the nucleic acid sequence SEQ ID NO. 146472, preferably the coding region thereof, conferred the production of or the increase in bis-glyceryl phosphate (polar fraction) compared with the wild type control.

The nucleic acid sequence of B3777 from Escherichia coli, e.g. as shown in the respective line in column 5 of Table I, application no. 19, has been published in B. And the activity of the gene product thereof is the activity of b3777-protein.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “b3777-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B3777, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B3777, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B3777, or a functional equivalent or a homolog thereof as depicted in column 8 of Table II, application no. 19, preferably a homolog or functional equivalent as depicted in column 8 of Table II B, application no. 19, and being depicted in the same respective line as said B3777, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “b3777-protein”, preferably it is the molecule of section (a) or (b) of this paragraph.

In particular, it was observed that in plants, especially in Arabidopsis thaliana, increasing or generating the activity of a gene product non-targeted with the activity of a “b3777-protein”, preferably being encoded by a gene comprising the nucleic acid sequence SEQ ID NO. 136098, preferably the coding region thereof, conferred the production of or the increase in bis-glyceryl phosphate (polar fraction) compared with the wild type control.

The nucleic acid sequence of B3791 from Escherichia coli, e.g. as shown in the respective line in column 5 of Table I, application no. 19, has been published in B. And the activity of the gene product thereof is the activity of b3791-protein.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “b3791-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B3791, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B3791, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B3791, or a functional equivalent or a homolog thereof as depicted in column 8 of Table II, application no. 19, preferably a homolog or functional equivalent as depicted in column 8 of Table II B, application no. 19, and being depicted in the same respective line as said B3791, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “b3791-protein”, preferably it is the molecule of section (a) or (b) of this paragraph.

In particular, it was observed that in plants, especially in Arabidopsis thaliana, increasing or generating the activity of a gene product non-targeted with the activity of a “b3791-protein”, preferably being encoded by a gene comprising the nucleic acid sequence SEQ ID NO. 83726, preferably the coding region thereof, conferred the production of or the increase in bis-glyceryl phosphate (polar fraction) compared with the wild type control.

The nucleic acid sequence of B3793 from Escherichia coli, e.g. as shown in the respective line in column 5 of Table I, application no. 19, has been published in B. And the activity of the gene product thereof is the activity of enterobacterial common antigen polymerase.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “enterobacterial common antigen polymerase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B3793, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B3793, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B3793, or a functional equivalent or a homolog thereof as depicted in column 8 of Table II, application no. 19, preferably a homolog or functional equivalent as depicted in column 8 of Table II B, application no. 19, and being depicted in the same respective line as said B3793, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “enterobacterial common antigen polymerase”, preferably it is the molecule of section (a) or (b) of this paragraph.

In particular, it was observed that in plants, especially in Arabidopsis thaliana, increasing or generating the activity of a gene product non-targeted with the activity of a “enterobacterial common antigen polymerase”, preferably being encoded by a gene comprising the nucleic acid sequence SEQ ID NO. 43800, preferably the coding region thereof, conferred the production of or the increase in bis-glyceryl phosphate (polar fraction) compared with the wild type control.

The nucleic acid sequence of B3989 from Escherichia coli, e.g. as shown in the respective line in column 5 of Table I, application no. 19, has been published in B. And the activity of the gene product thereof is the activity of b3989-protein.

Accordingly, in one embodiment, the process of the present invention for producing triacylglycerol in a non-human organism, like a microorganism or a plant or a part thereof, comprises increasing or generating the activity of a gene product with the activity of a gene product conferring the activity of “b3989-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical triacylglycerol), application no. 19, preferably the coding region thereof, or a homolog or a fragment thereof, and being depicted in the same respective line as said B3989, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B3989, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B3989, or a functional equivalent or a homolog thereof as depicted in column 8 of Table II, application no. 19, preferably a homolog or functional equivalent as depicted in column 8 of Table II B, application no. 19, and being depicted in the same respective line as said B3989, and preferably the activity is increased non-targeted, whereby the respective line disclose in column 7 the fine chemical triacylglycerol.

Accordingly, in one embodiment, the molecule which activity is to be increased in the process of the invention is the gene product with an activity as a “b3989-protein”, preferably it is the molecule of section (a) or (b) of this paragraph.

In particular, it was observed that in plants, especially in Arabidopsis thaliana, increasing or generating the activity of a gene product non-targeted with the activity of a “b3989-protein”, preferably being encoded by a gene comprising the nucleic acid sequence SEQ ID NO. 44372, preferably the coding region thereof, conferred the production of or the increase in triacylglycerol compared with the wild type control.

The nucleic acid sequence of B4029 from Escherichia coli, e.g. as shown in the respective line in column 5 of Table I, application no. 19, has been published in B. And the activity of the gene product thereof is the activity of b4029-protein.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “b4029-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B4029, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B4029, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B4029, or a functional equivalent or a homolog thereof as depicted in column 8 of Table II, application no. 19, preferably a homolog or functional equivalent as depicted in column 8 of Table II B, application no. 19, and being depicted in the same respective line as said B4029, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “b4029-protein”, preferably it is the molecule of section (a) or (b) of this paragraph.

In particular, it was observed that in plants, especially in Arabidopsis thaliana, increasing or generating the activity of a gene product non-targeted with the activity of a “b4029-protein”, preferably being encoded by a gene comprising the nucleic acid sequence SEQ ID NO. 10740, preferably the coding region thereof, conferred the production of or the increase in bis-glyceryl phosphate (polar fraction) compared with the wild type control.

The nucleic acid sequence of B4050 from Escherichia coli, e.g. as shown in the respective line in column 5 of Table I, application no. 19, has been published in B. And the activity of the gene product thereof is the activity of b4050-protein.

Accordingly, in one embodiment, the process of the present invention for producing triacylglycerol in a non-human organism, like a microorganism or a plant or a part thereof, comprises increasing or generating the activity of a gene product with the activity of a gene product conferring the activity of “b4050-protein”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical triacylglycerol), application no. 19, preferably the coding region thereof, or a homolog or a fragment thereof, and being depicted in the same respective line as said B4050, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B4050, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B4050, or a functional equivalent or a homolog thereof as depicted in column 8 of Table II, application no. 19, preferably a homolog or functional equivalent as depicted in column 8 of Table II B, application no. 19, and being depicted in the same respective line as said B4050, and preferably the activity is increased non-targeted, whereby the respective line disclose in column 7 the fine chemical triacylglycerol.

Accordingly, in one embodiment, the molecule which activity is to be increased in the process of the invention is the gene product with an activity as a “b4050-protein”, preferably it is the molecule of section (a) or (b) of this paragraph.

In particular, it was observed that in plants, especially in Arabidopsis thaliana, increasing or generating the activity of a gene product non-targeted with the activity of a “b4050-protein”, preferably being encoded by a gene comprising the nucleic acid sequence SEQ ID NO. 106532, preferably the coding region thereof, conferred the production of or the increase in triacylglycerol compared with the wild type control.

The nucleic acid sequence of B4129 from Escherichia coli, e.g. as shown in the respective line in column 5 of Table I, application no. 19, has been published in B. And the activity of the gene product thereof is the activity of lysyl-tRNA synthetase.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “lysyl-tRNA synthetase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said B4129, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B4129, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B4129, or a functional equivalent or a homolog thereof as depicted in column 8 of Table II, application no. 19, preferably a homolog or functional equivalent as depicted in column 8 of Table II B, application no. 19, and being depicted in the same respective line as said B4129, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “lysyl-tRNA synthetase”, preferably it is the molecule of section (a) or (b) of this paragraph.

In particular, it was observed that in plants, especially in Arabidopsis thaliana, increasing or generating the activity of a gene product non-targeted with the activity of a “lysyl-tRNA synthetase”, preferably being encoded by a gene comprising the nucleic acid sequence SEQ ID NO. 44662, preferably the coding region thereof, conferred the production of or the increase in bis-glyceryl phosphate (polar fraction) compared with the wild type control.

The nucleic acid sequence of B4384 from Escherichia coli, e.g. as shown in the respective line in column 5 of Table I, application no. 19, has been published in B. And the activity of the gene product thereof is the activity of purine-nucleoside phosphorylase.

Accordingly, in one embodiment, the process of the present invention for producing glycerol-3-phosphate (polar fraction) in a non-human organism, like a microorganism or a plant or a part thereof, comprises increasing or generating the activity of a gene product with the activity of a gene product conferring the activity of “purine-nucleoside phosphorylase”, especially from Escherichia coli or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol-3-phosphate (polar fraction)), application no. 19, preferably the coding region thereof, or a homolog or a fragment thereof, and being depicted in the same respective line as said B4384, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said B4384, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said B4384, or a functional equivalent or a homolog thereof as depicted in column 8 of Table II, application no. 19, preferably a homolog or functional equivalent as depicted in column 8 of Table II B, application no. 19, and being depicted in the same respective line as said B4384, and preferably the activity is increased non-targeted, whereby the respective line disclose in column 7 the fine chemical glycerol-3-phosphate (polar fraction).

Accordingly, in one embodiment, the molecule which activity is to be increased in the process of the invention is the gene product with an activity as a “purine-nucleoside phosphorylase”, preferably it is the molecule of section (a) or (b) of this paragraph.

In particular, it was observed that in plants, especially in Arabidopsis thaliana, increasing or generating the activity of a gene product non-targeted with the activity of a “purine-nucleoside phosphorylase”, preferably being encoded by a gene comprising the nucleic acid sequence SEQ ID NO. 45556, preferably the coding region thereof, conferred the production of or the increase in glycerol-3-phosphate (polar fraction) compared with the wild type control.

The nucleic acid sequence of GM02LC12622 from Glycine max, e.g. as shown in the respective line in column 5 of Table I, application no. 19, is unpublished. And the activity of the gene product thereof is the activity of glutaredoxin.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “glutaredoxin”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said GM02LC12622, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said GM02LC12622, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said GM02LC12622, or a functional equivalent or a homolog thereof as depicted in column 8 of Table II, application no. 19, preferably a homolog or functional equivalent as depicted in column 8 of Table II B, application no. 19, and being depicted in the same respective line as said GM02LC12622, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 10811, preferably the coding region thereof, conferred the production of or the increase in bis-glyceryl phosphate (polar fraction) compared with the wild type control.

The nucleic acid sequence of GM02LC12622 from Glycine max, e.g. as shown in the respective line in column 5 of Table I, application no. 19, is unpublished. And the activity of the gene product thereof is the activity of glutaredoxin.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “glutaredoxin”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said GM02LC12622, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said GM02LC12622, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said GM02LC12622, or a functional equivalent or a homolog thereof as depicted in column 8 of Table II, application no. 19, preferably a homolog or functional equivalent as depicted in column 8 of Table II B, application no. 19, and being depicted in the same respective line as said GM02LC12622, and preferably the activity is increased non-targeted, whereby the respective line disclose in column 7 the fine chemical glycerol phosphate (lipid fraction).

Accordingly, in one embodiment, the molecule which activity is to be increased in the process of the invention is the gene product with an activity as a “glutaredoxin”, preferably it is the molecule of section (a) or (b) of this paragraph.

In particular, it was observed that in plants, especially in Arabidopsis thaliana, increasing or generating the activity of a gene product non-targeted with the activity of a “glutaredoxin”, preferably being encoded by a gene comprising the nucleic acid sequence SEQ ID NO. 10811, preferably the coding region thereof, conferred the production of or the increase in glycerol phosphate (lipid fraction) compared with the wild type control.

The nucleic acid sequence of GM02LC17485 from Glycine max, e.g. as shown in the respective line in column 5 of Table I, application no. 19, is unpublished. And the activity of the gene product thereof is the activity of GM02LC17485-protein.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “GM02LC17485-protein”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said GM02LC17485, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said GM02LC17485, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said GM02LC17485, or a functional equivalent or a homolog thereof as depicted in column 8 of Table II, application no. 19, preferably a homolog or functional equivalent as depicted in column 8 of Table II B, application no. 19, and being depicted in the same respective line as said GM02LC17485, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “GM02LC17485-protein”, preferably it is the molecule of section (a) or (b) of this paragraph.

In particular, it was observed that in plants, especially in Arabidopsis thaliana, increasing or generating the activity of a gene product non-targeted with the activity of a “GM02LC17485-protein”, preferably being encoded by a gene comprising the nucleic acid sequence SEQ ID NO. 46405, preferably the coding region thereof, conferred the production of or the increase in bis-glyceryl phosphate (polar fraction) compared with the wild type control.

The nucleic acid sequence of GM02LC19289 from Glycine max, e.g. as shown in the respective line in column 5 of Table I, application no. 19, is unpublished. And the activity of the gene product thereof is the activity of transcription factor.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said GM02LC19289, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said GM02LC19289, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said GM02LC19289, or a functional equivalent or a homolog thereof as depicted in column 8 of Table II, application no. 19, preferably a homolog or functional equivalent as depicted in column 8 of Table II B, application no. 19, and being depicted in the same respective line as said GM02LC19289, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 46751, preferably the coding region thereof, conferred the production of or the increase in glycerol (lipid fraction) compared with the wild type control.

The nucleic acid sequence of GM02LC19289 from Glycine max, e.g. as shown in the respective line in column 5 of Table I, application no. 19, is unpublished. And the activity of the gene product thereof is the activity of transcription factor.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said GM02LC19289, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said GM02LC19289, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said GM02LC19289, or a functional equivalent or a homolog thereof as depicted in column 8 of Table II, application no. 19, preferably a homolog or functional equivalent as depicted in column 8 of Table II B, application no. 19, and being depicted in the same respective line as said GM02LC19289, and preferably the activity is increased non-targeted, whereby the respective line disclose in column 7 the fine chemical glycerol phosphate (lipid fraction).

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 46751, preferably the coding region thereof, conferred the production of or the increase in glycerol phosphate (lipid fraction) compared with the wild type control.

The nucleic acid sequence of GM02LC21368 from Glycine max, e.g. as shown in the respective line in column 5 of Table I, application no. 19, is unpublished. And the activity of the gene product thereof is the activity of transcription factor.

Accordingly, in one embodiment, the process of the present invention for producing glycerol (polar fraction) in a non-human organism, like a microorganism or a plant or a part thereof, comprises increasing or generating the activity of a gene product with the activity of a gene product conferring the activity of “transcription factor”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (polar fraction)), application no. 19, preferably the coding region thereof, or a homolog or a fragment thereof, and being depicted in the same respective line as said GM02LC21368, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said GM02LC21368, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said GM02LC21368, or a functional equivalent or a homolog thereof as depicted in column 8 of Table II, application no. 19, preferably a homolog or functional equivalent as depicted in column 8 of Table II B, application no. 19, and being depicted in the same respective line as said GM02LC21368, and preferably the activity is increased non-targeted, whereby the respective line disclose in column 7 the fine chemical glycerol (polar fraction).

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 84198, preferably the coding region thereof, conferred the production of or the increase in glycerol (polar fraction) compared with the wild type control.

The nucleic acid sequence of GM02LC44512 from Glycine max, e.g. as shown in the respective line in column 5 of Table I, application no. 19, is unpublished. And the activity of the gene product thereof is the activity of cyclin D.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “cyclin D”, especially from Glycine max or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said GM02LC44512, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said GM02LC44512, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said GM02LC44512, or a functional equivalent or a homolog thereof as depicted in column 8 of Table II, application no. 19, preferably a homolog or functional equivalent as depicted in column 8 of Table II B, application no. 19, and being depicted in the same respective line as said GM02LC44512, and preferably the activity is increased non-targeted, whereby the respective line disclose in column 7 the fine chemical glycerol phosphate (lipid fraction).

Accordingly, in one embodiment, the molecule which activity is to be increased in the process of the invention is the gene product with an activity as a “cyclin D”, preferably it is the molecule of section (a) or (b) of this paragraph.

In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 46850, preferably the coding region thereof, conferred the production of or the increase in glycerol phosphate (lipid fraction) compared with the wild type control.

The nucleic acid sequence of Sll1450 from Synechocystis sp., e.g. as shown in the respective line in column 5 of Table I, application no. 19, has been published in C. And the activity of the gene product thereof is the activity of nitrate/nitrite transport protein.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “nitrate/nitrite transport protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said Sll1450, or a functional equivalent or a homolog thereof as shown in column 8 of Table 1, application no. 19, preferably the coding region thereof, particularly a homolog or functional equivalent as shown in column 8 of Table I B, application no. 19, and being depicted in the same respective line as said Sll1450, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Sll1450, or a functional equivalent or a homolog thereof as depicted in column 8 of Table II, application no. 19, preferably a homolog or functional equivalent as depicted in column 8 of Table 11 B, application no. 19, and being depicted in the same respective line as said Sll1450, and preferably the activity is increased mitochondrial, whereby the respective line disclose in column 7 the fine chemical bis-glyceryl phosphate (polar fraction).

Accordingly, in one embodiment, the molecule which activity is to be increased in the process of the invention is the gene product with an activity as a “nitrate/nitrite transport protein”, preferably it is the molecule of section (a) or (b) of this paragraph.

In particular, it was observed that in plants, especially in Arabidopsis thaliana, increasing or generating the activity of a gene product mitochondrial with the activity of a “nitrate/nitrite transport protein”, preferably being encoded by a gene comprising the nucleic acid sequence SEQ ID NO. 54337, preferably the coding region thereof, conferred the production of or the increase in bis-glyceryl phosphate (polar fraction) compared with the wild type control.

The nucleic acid sequence of Sll1498 from Synechocystis sp., e.g. as shown in the respective line in column 5 of Table I, application no. 19, has been published in C. And the activity of the gene product thereof is the activity of carbamoyl-phosphate synthase subunit.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “carbamoyl-phosphate synthase subunit”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said Sll1498, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Sll1498, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Sll1498, or a functional equivalent or a homolog thereof as depicted in column 8 of Table II, application no. 19, preferably a homolog or functional equivalent as depicted in column 8 of Table 11 B, application no. 19, and being depicted in the same respective line as said Sll1498, and preferably the activity is increased non-targeted, whereby the respective line disclose in column 7 the fine chemical glycerol phosphate (lipid fraction).

Accordingly, in one embodiment, the molecule which activity is to be increased in the process of the invention is the gene product with an activity as a “carbamoyl-phosphate synthase subunit”, preferably it is the molecule of section (a) or (b) of this paragraph.

In particular, it was observed that in plants, especially in Arabidopsis thaliana, increasing or generating the activity of a gene product non-targeted with the activity of a “carbamoylphosphate synthase subunit”, preferably being encoded by a gene comprising the nucleic acid sequence SEQ ID NO. 95047, preferably the coding region thereof, conferred the production of or the increase in glycerol phosphate (lipid fraction) compared with the wild type control.

The nucleic acid sequence of Sll1522 from Synechocystis sp., e.g. as shown in the respective line in column 5 of Table I, application no. 19, has been published in C. And the activity of the gene product thereof is the activity of CDP-diacylglycerol-glycerol-3-phosphate 3-phosphatidyltransferase.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “CDP-diacylglycerol-glycerol-3-phosphate 3-phosphatidyltransferase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said Sll1522, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Sll1522, and preferably the activity is increased plastidic, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Sll1522, or a functional equivalent or a homolog thereof as depicted in column 8 of Table II, application no. 19, preferably a homolog or functional equivalent as depicted in column 8 of Table 11 B, application no. 19, and being depicted in the same respective line as said Sll1522, and preferably the activity is increased plastidic, whereby the respective line disclose in column 7 the fine chemical glycerol (lipid fraction).

Accordingly, in one embodiment, the molecule which activity is to be increased in the process of the invention is the gene product with an activity as a “CDP-diacylglycerol-glycerol-3-phosphate 3-phosphatidyltransferase”, preferably it is the molecule of section (a) or (b) of this paragraph.

In particular, it was observed that in plants, especially in Arabidopsis thaliana, increasing or generating the activity of a gene product plastidic with the activity of a “CDP-diacylglycerolglycerol-3-phosphate 3-phosphatidyltransferase”, preferably being encoded by a gene comprising the nucleic acid sequence SEQ ID NO. 54452, preferably the coding region thereof, conferred the production of or the increase in glycerol (lipid fraction) compared with the wild type control.

The nucleic acid sequence of Sll1815 from Synechocystis sp., e.g. as shown in the respective line in column 5 of Table I, application no. 19, has been published in C. And the activity of the gene product thereof is the activity of adenylate kinase.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “adenylate kinase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said Sll1815, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Sll1815, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Sll1815, or a functional equivalent or a homolog thereof as depicted in column 8 of Table II, application no. 19, preferably a homolog or functional equivalent as depicted in column 8 of Table II B, application no. 19, and being depicted in the same respective line as said Sll1815, and preferably the activity is increased mitochondrial, whereby the respective line disclose in column 7 the fine chemical bis-glyceryl phosphate (polar fraction).

Accordingly, in one embodiment, the molecule which activity is to be increased in the process of the invention is the gene product with an activity as a “adenylate kinase”, preferably it is the molecule of section (a) or (b) of this paragraph.

In particular, it was observed that in plants, especially in Arabidopsis thaliana, increasing or generating the activity of a gene product mitochondrial with the activity of a “adenylate kinase”, preferably being encoded by a gene comprising the nucleic acid sequence SEQ ID NO. 85808, preferably the coding region thereof, conferred the production of or the increase in bis-glyceryl phosphate (polar fraction) compared with the wild type control.

The nucleic acid sequence of Sll1848 from Synechocystis sp., e.g. as shown in the respective line in column 5 of Table I, application no. 19, has been published in C. And the activity of the gene product thereof is the activity of acyl transferase.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “acyl transferase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said Sll1848, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Sll1848, and preferably the activity is increased plastidic, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Sll1848, or a functional equivalent or a homolog thereof as depicted in column 8 of Table II, application no. 19, preferably a homolog or functional equivalent as depicted in column 8 of Table 11 B, application no. 19, and being depicted in the same respective line as said Sll1848, and preferably the activity is increased plastidic, whereby the respective line disclose in column 7 the fine chemical glycerol (lipid fraction).

Accordingly, in one embodiment, the molecule which activity is to be increased in the process of the invention is the gene product with an activity as a “acyl transferase”, preferably it is the molecule of section (a) or (b) of this paragraph.

In particular, it was observed that in plants, especially in Arabidopsis thaliana, increasing or generating the activity of a gene product plastidic with the activity of a “acyl transferase”, preferably being encoded by a gene comprising the nucleic acid sequence SEQ ID NO. 86447, preferably the coding region thereof, conferred the production of or the increase in glycerol (lipid fraction) compared with the wild type control.

The nucleic acid sequence of Sll1917 from Synechocystis sp., e.g. as shown in the respective line in column 5 of Table I, application no. 19, has been published in C. And the activity of the gene product thereof is the activity of coproporphyrinogen III oxidase.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “coproporphyrinogen III oxidase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said Sll1917, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Sll1917, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Sll1917, or a functional equivalent or a homolog thereof as depicted in column 8 of Table II, application no. 19, preferably a homolog or functional equivalent as depicted in column 8 of Table II B, application no. 19, and being depicted in the same respective line as said Sll1917, and preferably the activity is increased mitochondrial, whereby the respective line disclose in column 7 the fine chemical glycerol phosphate (lipid fraction).

Accordingly, in one embodiment, the molecule which activity is to be increased in the process of the invention is the gene product with an activity as a “coproporphyrinogen III oxidase”, preferably it is the molecule of section (a) or (b) of this paragraph.

In particular, it was observed that in plants, especially in Arabidopsis thaliana, increasing or generating the activity of a gene product mitochondrial with the activity of a “coproporphyrinogen III oxidase”, preferably being encoded by a gene comprising the nucleic acid sequence SEQ ID NO. 11471, preferably the coding region thereof, conferred the production of or the increase in glycerol phosphate (lipid fraction) compared with the wild type control.

The nucleic acid sequence of Slr0338 from Synechocystis sp., e.g. as shown in the respective line in column 5 of Table I, application no. 19, has been published in C. And the activity of the gene product thereof is the activity of oxidoreductase.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “oxidoreductase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said Slr0338, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Slr0338, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Slr0338, or a functional equivalent or a homolog thereof as depicted in column 8 of Table II, application no. 19, preferably a homolog or functional equivalent as depicted in column 8 of Table II B, application no. 19, and being depicted in the same respective line as said Slr0338, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product non-targeted with the activity of a “oxidoreductase”, preferably being encoded by a gene comprising the nucleic acid sequence SEQ ID NO. 11990, preferably the coding region thereof, conferred the production of or the increase in glycerol (lipid fraction) compared with the wild type control.

The nucleic acid sequence of Slr0338 from Synechocystis sp., e.g. as shown in the respective line in column 5 of Table I, application no. 19, has been published in C. And the activity of the gene product thereof is the activity of oxidoreductase.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “oxidoreductase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said Slr0338, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Slr0338, and preferably the activity is increased plastidic, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Slr0338, or a functional equivalent or a homolog thereof as depicted in column 8 of Table II, application no. 19, preferably a homolog or functional equivalent as depicted in column 8 of Table II B, application no. 19, and being depicted in the same respective line as said Slr0338, and preferably the activity is increased plastidic, whereby the respective line disclose in column 7 the fine chemical glycerol phosphate (lipid fraction).

Accordingly, in one embodiment, the molecule which activity is to be increased in the process of the invention is the gene product with an activity as a “oxidoreductase”, preferably it is the molecule of section (a) or (b) of this paragraph.

In particular, it was observed that in plants, especially in Arabidopsis thaliana, increasing or generating the activity of a gene product plastidic with the activity of a “oxidoreductase”, preferably being encoded by a gene comprising the nucleic acid sequence SEQ ID NO. 11990, preferably the coding region thereof, conferred the production of or the increase in glycerol phosphate (lipid fraction) compared with the wild type control.

The nucleic acid sequence of Slr0905 from Synechocystis sp., e.g. as shown in the respective line in column 5 of Table I, application no. 19, has been published in C. And the activity of the gene product thereof is the activity of Mg-protoporphyrin IX monomethyl ester oxidative cyclase 66 kD subunit.

Accordingly, in one embodiment, the process of the present invention for producing glycerol (polar fraction) in a non-human organism, like a microorganism or a plant or a part thereof, comprises increasing or generating the activity of a gene product with the activity of a gene product conferring the activity of “Mg-protoporphyrin IX monomethyl ester oxidative cyclase 66 kD subunit”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (polar fraction)), application no. 19, preferably the coding region thereof, or a homolog or a fragment thereof, and being depicted in the same respective line as said Slr0905, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Slr0905, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Slr0905, or a functional equivalent or a homolog thereof as depicted in column 8 of Table II, application no. 19, preferably a homolog or functional equivalent as depicted in column 8 of Table II B, application no. 19, and being depicted in the same respective line as said Slr0905, and preferably the activity is increased mitochondrial, whereby the respective line disclose in column 7 the fine chemical glycerol (polar fraction).

Accordingly, in one embodiment, the molecule which activity is to be increased in the process of the invention is the gene product with an activity as a “Mg-protoporphyrin IX monomethyl ester oxidative cyclase 66 kD subunit”, preferably it is the molecule of section (a) or (b) of this paragraph.

In particular, it was observed that in plants, especially in Arabidopsis thaliana, increasing or generating the activity of a gene product mitochondrial with the activity of a “Mg-protoporphyrin IX monomethyl ester oxidative cyclase 66 kD subunit”, preferably being encoded by a gene comprising the nucleic acid sequence SEQ ID NO. 147407, preferably the coding region thereof, conferred the production of or the increase in glycerol (polar fraction) compared with the wild type control.

The nucleic acid sequence of Slr1096 from Synechocystis sp., e.g. as shown in the respective line in column 5 of Table I, application no. 19, has been published in C. And the activity of the gene product thereof is the activity of dihydrolipoamide dehydrogenase.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “dihydrolipoamide dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said Slr1096, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Slr1096, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Slr1096, or a functional equivalent or a homolog thereof as depicted in column 8 of Table II, application no. 19, preferably a homolog or functional equivalent as depicted in column 8 of Table II B, application no. 19, and being depicted in the same respective line as said Slr1096, and preferably the activity is increased non-targeted, whereby the respective line disclose in column 7 the fine chemical bis-glyceryl phosphate (polar fraction).

Accordingly, in one embodiment, the molecule which activity is to be increased in the process of the invention is the gene product with an activity as a “dihydrolipoamide dehydrogenase”, preferably it is the molecule of section (a) or (b) of this paragraph.

In particular, it was observed that in plants, especially in Arabidopsis thaliana, increasing or generating the activity of a gene product non-targeted with the activity of a “dihydrolipoamide dehydrogenase”, preferably being encoded by a gene comprising the nucleic acid sequence SEQ ID NO. 57734, preferably the coding region thereof, conferred the production of or the increase in bis-glyceryl phosphate (polar fraction) compared with the wild type control.

The nucleic acid sequence of Slr1099 from Synechocystis sp., e.g. as shown in the respective line in column 5 of Table I, application no. 19, has been published in C. And the activity of the gene product thereof is the activity of aromatic acid decarboxylase.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “aromatic acid decarboxylase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said Slr1099, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Slr1099, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Slr1099, or a functional equivalent or a homolog thereof as depicted in column 8 of Table II, application no. 19, preferably a homolog or functional equivalent as depicted in column 8 of Table II B, application no. 19, and being depicted in the same respective line as said Slr1099, and preferably the activity is increased mitochondrial, whereby the respective line disclose in column 7 the fine chemical glycerol phosphate (lipid fraction).

Accordingly, in one embodiment, the molecule which activity is to be increased in the process of the invention is the gene product with an activity as a “aromatic acid decarboxylase”, preferably it is the molecule of section (a) or (b) of this paragraph.

In particular, it was observed that in plants, especially in Arabidopsis thaliana, increasing or generating the activity of a gene product mitochondrial with the activity of a “aromatic acid decarboxylase”, preferably being encoded by a gene comprising the nucleic acid sequence SEQ ID NO. 116341, preferably the coding region thereof, conferred the production of or the increase in glycerol phosphate (lipid fraction) compared with the wild type control.

The nucleic acid sequence of Slr1250 from Synechocystis sp., e.g. as shown in the respective line in column 5 of Table I, application no. 19, has been published in C. And the activity of the gene product thereof is the activity of phosphate import ATP-binding protein.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “phosphate import ATP-binding protein”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said Slr1250, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Slr1250, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Slr1250, or a functional equivalent or a homolog thereof as depicted in column 8 of Table II, application no. 19, preferably a homolog or functional equivalent as depicted in column 8 of Table II B, application no. 19, and being depicted in the same respective line as said Slr1250, and preferably the activity is increased mitochondrial, whereby the respective line disclose in column 7 the fine chemical glycerol phosphate (lipid fraction).

Accordingly, in one embodiment, the molecule which activity is to be increased in the process of the invention is the gene product with an activity as a “phosphate import ATP-binding protein”, preferably it is the molecule of section (a) or (b) of this paragraph.

In particular, it was observed that in plants, especially in Arabidopsis thaliana, increasing or generating the activity of a gene product mitochondrial with the activity of a “phosphate import ATP-binding protein”, preferably being encoded by a gene comprising the nucleic acid sequence SEQ ID NO. 116460, preferably the coding region thereof, conferred the production of or the increase in glycerol phosphate (lipid fraction) compared with the wild type control.

The nucleic acid sequence of Slr1312 from Synechocystis sp., e.g. as shown in the respective line in column 5 of Table I, application no. 19, has been published in C. And the activity of the gene product thereof is the activity of arginine decarboxylase.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “arginine decarboxylase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said Slr1312, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Slr1312, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Slr1312, or a functional equivalent or a homolog thereof as depicted in column 8 of Table II, application no. 19, preferably a homolog or functional equivalent as depicted in column 8 of Table II B, application no. 19, and being depicted in the same respective line as said Slr1312, and preferably the activity is increased non-targeted, whereby the respective line disclose in column 7 the fine chemical glycerol phosphate (lipid fraction).

Accordingly, in one embodiment, the molecule which activity is to be increased in the process of the invention is the gene product with an activity as a “arginine decarboxylase”, preferably it is the molecule of section (a) or (b) of this paragraph.

In particular, it was observed that in plants, especially in Arabidopsis thaliana, increasing or generating the activity of a gene product non-targeted with the activity of a “arginine decarboxylase”, preferably being encoded by a gene comprising the nucleic acid sequence SEQ ID NO. 58324, preferably the coding region thereof, conferred the production of or the increase in glycerol phosphate (lipid fraction) compared with the wild type control.

The nucleic acid sequence of Slr1420 from Synechocystis sp., e.g. as shown in the respective line in column 5 of Table I, application no. 19, has been published in C. And the activity of the gene product thereof is the activity of carbohydrate kinase.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “carbohydrate kinase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said Slr1420, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Slr1420, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Slr1420, or a functional equivalent or a homolog thereof as depicted in column 8 of Table II, application no. 19, preferably a homolog or functional equivalent as depicted in column 8 of Table II B, application no. 19, and being depicted in the same respective line as said Slr1420, and preferably the activity is increased non-targeted, whereby the respective line disclose in column 7 the fine chemical glycerol phosphate (lipid fraction).

Accordingly, in one embodiment, the molecule which activity is to be increased in the process of the invention is the gene product with an activity as a “carbohydrate kinase”, preferably it is the molecule of section (a) or (b) of this paragraph.

In particular, it was observed that in plants, especially in Arabidopsis thaliana, increasing or generating the activity of a gene product non-targeted with the activity of a “carbohydrate kinase”, preferably being encoded by a gene comprising the nucleic acid sequence SEQ ID NO. 58590, preferably the coding region thereof, conferred the production of or the increase in glycerol phosphate (lipid fraction) compared with the wild type control.

The nucleic acid sequence of Slr1467 from Synechocystis sp., e.g. as shown in the respective line in column 5 of Table I, application no. 19, has been published in C. And the activity of the gene product thereof is the activity of precorrin-8X methylmutase.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “precorrin-8X methylmutase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said Slr1467, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Slr1467, and preferably the activity is increased plastidic, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Slr1467, or a functional equivalent or a homolog thereof as depicted in column 8 of Table II, application no. 19, preferably a homolog or functional equivalent as depicted in column 8 of Table II B, application no. 19, and being depicted in the same respective line as said Slr1467, and preferably the activity is increased plastidic, whereby the respective line disclose in column 7 the fine chemical bis-glyceryl phosphate (polar fraction).

Accordingly, in one embodiment, the molecule which activity is to be increased in the process of the invention is the gene product with an activity as a “precorrin-8X methylmutase”, preferably it is the molecule of section (a) or (b) of this paragraph.

In particular, it was observed that in plants, especially in Arabidopsis thaliana, increasing or generating the activity of a gene product plastidic with the activity of a “precorrin-8X methylmutase”, preferably being encoded by a gene comprising the nucleic acid sequence SEQ ID NO. 154137, preferably the coding region thereof, conferred the production of or the increase in bis-glyceryl phosphate (polar fraction) compared with the wild type control.

The nucleic acid sequence of Slr1743 from Synechocystis sp., e.g. as shown in the respective line in column 5 of Table I, application no. 19, has been published in C. And the activity of the gene product thereof is the activity of NADH dehydrogenase.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “NADH dehydrogenase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said Slr1743, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Slr1743, and preferably the activity is increased plastidic, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Slr1743, or a functional equivalent or a homolog thereof as depicted in column 8 of Table II, application no. 19, preferably a homolog or functional equivalent as depicted in column 8 of Table II B, application no. 19, and being depicted in the same respective line as said Slr1743, and preferably the activity is increased plastidic, whereby the respective line disclose in column 7 the fine chemical bis-glyceryl phosphate (polar fraction).

Accordingly, in one embodiment, the molecule which activity is to be increased in the process of the invention is the gene product with an activity as a “NADH dehydrogenase”, preferably it is the molecule of section (a) or (b) of this paragraph.

In particular, it was observed that in plants, especially in Arabidopsis thaliana, increasing or generating the activity of a gene product plastidic with the activity of a “NADH dehydrogenase”, preferably being encoded by a gene comprising the nucleic acid sequence SEQ ID NO. 147457, preferably the coding region thereof, conferred the production of or the increase in bis-glyceryl phosphate (polar fraction) compared with the wild type control.

The nucleic acid sequence of Slr2035 from Synechocystis sp., e.g. as shown in the respective line in column 5 of Table I, application no. 19, has been published in C. And the activity of the gene product thereof is the activity of glutamate 5-kinase.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “glutamate 5-kinase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said Slr2035, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Slr2035, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Slr2035, or a functional equivalent or a homolog thereof as depicted in column 8 of Table II, application no. 19, preferably a homolog or functional equivalent as depicted in column 8 of Table II B, application no. 19, and being depicted in the same respective line as said Slr2035, and preferably the activity is increased mitochondrial, whereby the respective line disclose in column 7 the fine chemical glycerol (lipid fraction).

Accordingly, in one embodiment, the molecule which activity is to be increased in the process of the invention is the gene product with an activity as a “glutamate 5-kinase”, preferably it is the molecule of section (a) or (b) of this paragraph.

In particular, it was observed that in plants, especially in Arabidopsis thaliana, increasing or generating the activity of a gene product mitochondrial with the activity of a “glutamate 5-kinase”, preferably being encoded by a gene comprising the nucleic acid sequence SEQ ID NO. 127521, preferably the coding region thereof, conferred the production of or the increase in glycerol (lipid fraction) compared with the wild type control.

The nucleic acid sequence of Slr2072 from Synechocystis sp., e.g. as shown in the respective line in column 5 of Table I, application no. 19, has been published in C. And the activity of the gene product thereof is the activity of threonine dehydratase.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “threonine dehydratase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said Slr2072, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Slr2072, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Slr2072, or a functional equivalent or a homolog thereof as depicted in column 8 of Table II, application no. 19, preferably a homolog or functional equivalent as depicted in column 8 of Table II B, application no. 19, and being depicted in the same respective line as said Slr2072, and preferably the activity is increased mitochondrial, whereby the respective line disclose in column 7 the fine chemical glycerol (lipid fraction).

Accordingly, in one embodiment, the molecule which activity is to be increased in the process of the invention is the gene product with an activity as a “threonine dehydratase”, preferably it is the molecule of section (a) or (b) of this paragraph.

In particular, it was observed that in plants, especially in Arabidopsis thaliana, increasing or generating the activity of a gene product mitochondrial with the activity of a “threonine dehydratase”, preferably being encoded by a gene comprising the nucleic acid sequence SEQ ID NO. 12341, preferably the coding region thereof, conferred the production of or the increase in glycerol (lipid fraction) compared with the wild type control.

The nucleic acid sequence of Slr2072 from Synechocystis sp., e.g. as shown in the respective line in column 5 of Table I, application no. 19, has been published in C. And the activity of the gene product thereof is the activity of threonine dehydratase.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “threonine dehydratase”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said Slr2072, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Slr2072, and preferably the activity is increased mitochondrial, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Slr2072, or a functional equivalent or a homolog thereof as depicted in column 8 of Table II, application no. 19, preferably a homolog or functional equivalent as depicted in column 8 of Table II B, application no. 19, and being depicted in the same respective line as said Slr2072, and preferably the activity is increased mitochondrial, whereby the respective line disclose in column 7 the fine chemical glycerol phosphate (lipid fraction).

Accordingly, in one embodiment, the molecule which activity is to be increased in the process of the invention is the gene product with an activity as a “threonine dehydratase”, preferably it is the molecule of section (a) or (b) of this paragraph.

In particular, it was observed that in plants, especially in Arabidopsis thaliana, increasing or generating the activity of a gene product mitochondrial with the activity of a “threonine dehydratase”, preferably being encoded by a gene comprising the nucleic acid sequence SEQ ID NO. 12341, preferably the coding region thereof, conferred the production of or the increase in glycerol phosphate (lipid fraction) compared with the wild type control.

The nucleic acid sequence of Smr0004 from Synechocystis sp., e.g. as shown in the respective line in column 5 of Table I, application no. 19, has been published in C. And the activity of the gene product thereof is the activity of photosystem I reaction centre subunit.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “photosystem I reaction centre subunit”, especially from Synechocystis sp. or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said Smr0004, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Smr0004, and preferably the activity is increased plastidic, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Smr0004, or a functional equivalent or a homolog thereof as depicted in column 8 of Table II, application no. 19, preferably a homolog or functional equivalent as depicted in column 8 of Table II B, application no. 19, and being depicted in the same respective line as said Smr0004, and preferably the activity is increased plastidic, whereby the respective line disclose in column 7 the fine chemical bis-glyceryl phosphate (polar fraction).

Accordingly, in one embodiment, the molecule which activity is to be increased in the process of the invention is the gene product with an activity as a “photosystem I reaction centre subunit”, preferably it is the molecule of section (a) or (b) of this paragraph.

In particular, it was observed that in plants, especially in Arabidopsis thaliana, increasing or generating the activity of a gene product plastidic with the activity of a “photosystem I reaction centre subunit”, preferably being encoded by a gene comprising the nucleic acid sequence SEQ ID NO. 136907, preferably the coding region thereof, conferred the production of or the increase in bis-glyceryl phosphate (polar fraction) compared with the wild type control.

The nucleic acid sequence of TTC0035 from Thermus thermophilus, e.g. as shown in the respective line in column 5 of Table I, application no. 19, has been published in E. And the activity of the gene product thereof is the activity of cell division protein.

Accordingly, in one embodiment, the process of the present invention for producing triacylglycerol in a non-human organism, like a microorganism or a plant or a part thereof, comprises increasing or generating the activity of a gene product with the activity of a gene product conferring the activity of “cell division protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical triacylglycerol), application no. 19, preferably the coding region thereof, or a homolog or a fragment thereof, and being depicted in the same respective line as said TTC0035, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said TTC0035, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said TTC0035, or a functional equivalent or a homolog thereof as depicted in column 8 of Table II, application no. 19, preferably a homolog or functional equivalent as depicted in column 8 of Table II B, application no. 19, and being depicted in the same respective line as said TTC0035, and preferably the activity is increased non-targeted, whereby the respective line disclose in column 7 the fine chemical triacylglycerol.

Accordingly, in one embodiment, the molecule which activity is to be increased in the process of the invention is the gene product with an activity as a “cell division protein”, preferably it is the molecule of section (a) or (b) of this paragraph.

In particular, it was observed that in plants, especially in Arabidopsis thaliana, increasing or generating the activity of a gene product non-targeted with the activity of a “cell division protein”, preferably being encoded by a gene comprising the nucleic acid sequence SEQ ID NO. 60301, preferably the coding region thereof, conferred the production of or the increase in triacylglycerol compared with the wild type control.

The nucleic acid sequence of TTC0917 from Thermus thermophilus, e.g. as shown in the respective line in column 5 of Table I, application no. 19, has been published in E. And the activity of the gene product thereof is the activity of metal-dependent hydrolase.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “metal-dependent hydrolase”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said TTC0917, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said TTC0917, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said TTC0917, or a functional equivalent or a homolog thereof as depicted in column 8 of Table II, application no. 19, preferably a homolog or functional equivalent as depicted in column 8 of Table II B, application no. 19, and being depicted in the same respective line as said TTC0917, and preferably the activity is increased non-targeted, whereby the respective line disclose in column 7 the fine chemical glycerol phosphate (lipid fraction).

Accordingly, in one embodiment, the molecule which activity is to be increased in the process of the invention is the gene product with an activity as a “metal-dependent hydrolase”, preferably it is the molecule of section (a) or (b) of this paragraph.

In particular, it was observed that in plants, especially in Arabidopsis thaliana, increasing or generating the activity of a gene product non-targeted with the activity of a “metal-dependent hydrolase”, preferably being encoded by a gene comprising the nucleic acid sequence SEQ ID NO. 61553, preferably the coding region thereof, conferred the production of or the increase in glycerol phosphate (lipid fraction) compared with the wild type control.

The nucleic acid sequence of TTC1386 from Thermus thermophilus, e.g. as shown in the respective line in column 5 of Table I, application no. 19, has been published in E. And the activity of the gene product thereof is the activity of TTC1386-protein.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “TTC1386-protein”, especially from Thermus thermophilus or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said TTC1386, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said TTC1386, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said TTC1386, or a functional equivalent or a homolog thereof as depicted in column 8 of Table II, application no. 19, preferably a homolog or functional equivalent as depicted in column 8 of Table II B, application no. 19, and being depicted in the same respective line as said TTC1386, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “TTC1386-protein”, preferably it is the molecule of section (a) or (b) of this paragraph.

In particular, it was observed that in plants, especially in Arabidopsis thaliana, increasing or generating the activity of a gene product non-targeted with the activity of a “TTC1386-protein”, preferably being encoded by a gene comprising the nucleic acid sequence SEQ ID NO. 62079, preferably the coding region thereof, conferred the production of or the increase in glycerol (lipid fraction) compared with the wild type control.

The nucleic acid sequence of Yal013w from Saccharomyces cerevisiae, e.g. as shown in the respective line in column 5 of Table I, application no. 19, has been published in A. And the activity of the gene product thereof is the activity of YAL013W-protein.

Accordingly, in one embodiment, the process of the present invention for producing glycerol-3-phosphate (polar fraction) in a non-human organism, like a microorganism or a plant or a part thereof, comprises increasing or generating the activity of a gene product with the activity of a gene product conferring the activity of “YAL013W-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol-3-phosphate (polar fraction)), application no. 19, preferably the coding region thereof, or a homolog or a fragment thereof, and being depicted in the same respective line as said Yal013w, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Yal013w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Yal013w, or a functional equivalent or a homolog thereof as depicted in column 8 of Table II, application no. 19, preferably a homolog or functional equivalent as depicted in column 8 of Table II B, application no. 19, and being depicted in the same respective line as said Yal013w, and preferably the activity is increased non-targeted, whereby the respective line disclose in column 7 the fine chemical glycerol-3-phosphate (polar fraction).

Accordingly, in one embodiment, the molecule which activity is to be increased in the process of the invention is the gene product with an activity as a “YAL013W-protein”, preferably it is the molecule of section (a) or (b) of this paragraph.

In particular, it was observed that in plants, especially in Arabidopsis thaliana, increasing or generating the activity of a gene product non-targeted with the activity of a “YAL013W-protein”, preferably being encoded by a gene comprising the nucleic acid sequence SEQ ID NO. 108092, preferably the coding region thereof, conferred the production of or the increase in glycerol-3-phosphate (polar fraction) compared with the wild type control.

The nucleic acid sequence of Ybl021c from Saccharomyces cerevisiae, e.g. as shown in the respective line in column 5 of Table I, application no. 19, has been published in A. And the activity of the gene product thereof is the activity of transcription factor.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “transcription factor”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said Ybl021c, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Ybl021c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Ybl021c, or a functional equivalent or a homolog thereof as depicted in column 8 of Table II, application no. 19, preferably a homolog or functional equivalent as depicted in column 8 of Table II B, application no. 19, and being depicted in the same respective line as said Ybl021c, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “transcription factor”, preferably it is the molecule of section (a) or (b) of this paragraph.

In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 62524, preferably the coding region thereof, conferred the production of or the increase in glycerol (lipid fraction) compared with the wild type control.

The nucleic acid sequence of Ybr092c from Saccharomyces cerevisiae, e.g. as shown in the respective line in column 5 of Table I, application no. 19, has been published in A. And the activity of the gene product thereof is the activity of acid phosphatase precursor.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “acid phosphatase precursor”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said Ybr092c, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Ybr092c, and preferably the activity is increased plastidic, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Ybr092c, or a functional equivalent or a homolog thereof as depicted in column 8 of Table II, application no. 19, preferably a homolog or functional equivalent as depicted in column 8 of Table II B, application no. 19, and being depicted in the same respective line as said Ybr092c, and preferably the activity is increased plastidic, whereby the respective line disclose in column 7 the fine chemical glycerol phosphate (lipid fraction).

Accordingly, in one embodiment, the molecule which activity is to be increased in the process of the invention is the gene product with an activity as a “acid phosphatase precursor”, preferably it is the molecule of section (a) or (b) of this paragraph.

In particular, it was observed that in plants, especially in Arabidopsis thaliana, increasing or generating the activity of a gene product plastidic with the activity of a “acid phosphatase precursor”, preferably being encoded by a gene comprising the nucleic acid sequence SEQ ID NO. 119347, preferably the coding region thereof, conferred the production of or the increase in glycerol phosphate (lipid fraction) compared with the wild type control.

The nucleic acid sequence of Ybr160w from Saccharomyces cerevisiae, e.g. as shown in the respective line in column 5 of Table I, application no. 19, has been published in A. And the activity of the gene product thereof is the activity of cell division control protein.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “cell division control protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said Ybr160w, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Ybr160w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Ybr160w, or a functional equivalent or a homolog thereof as depicted in column 8 of Table II, application no. 19, preferably a homolog or functional equivalent as depicted in column 8 of Table II B, application no. 19, and being depicted in the same respective line as said Ybr160w, and preferably the activity is increased non-targeted, whereby the respective line disclose in column 7 the fine chemical glycerol (lipid fraction).

Accordingly, in one embodiment, the molecule which activity is to be increased in the process of the invention is the gene product with an activity as a “cell division control protein”, preferably it is the molecule of section (a) or (b) of this paragraph.

In particular, it was observed that in plants, especially in Arabidopsis thaliana, increasing or generating the activity of a gene product non-targeted with the activity of a “cell division control protein”, preferably being encoded by a gene comprising the nucleic acid sequence SEQ ID NO. 62717, preferably the coding region thereof, conferred the production of or the increase in glycerol (lipid fraction) compared with the wild type control.

The nucleic acid sequence of Ybr160w from Saccharomyces cerevisiae, e.g. as shown in the respective line in column 5 of Table I, application no. 19, has been published in A. And the activity of the gene product thereof is the activity of cell division control protein.

Accordingly, in one embodiment, the process of the present invention for producing glycerol (polar fraction) in a non-human organism, like a microorganism or a plant or a part thereof, comprises increasing or generating the activity of a gene product with the activity of a gene product conferring the activity of “cell division control protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (polar fraction)), application no. 19, preferably the coding region thereof, or a homolog or a fragment thereof, and being depicted in the same respective line as said Ybr160w, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Ybr160w, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Ybr160w, or a functional equivalent or a homolog thereof as depicted in column 8 of Table II, application no. 19, preferably a homolog or functional equivalent as depicted in column 8 of Table II B, application no. 19, and being depicted in the same respective line as said Ybr160w, and preferably the activity is increased non-targeted, whereby the respective line disclose in column 7 the fine chemical glycerol (polar fraction).

Accordingly, in one embodiment, the molecule which activity is to be increased in the process of the invention is the gene product with an activity as a “cell division control protein”, preferably it is the molecule of section (a) or (b) of this paragraph.

In particular, it was observed that in plants, especially in Arabidopsis thaliana, increasing or generating the activity of a gene product non-targeted with the activity of a “cell division control protein”, preferably being encoded by a gene comprising the nucleic acid sequence SEQ ID NO. 62717, preferably the coding region thereof, conferred the production of or the increase in glycerol (polar fraction) compared with the wild type control.

The nucleic acid sequence of Ycr102c from Saccharomyces cerevisiae, e.g. as shown in the respective line in column 5 of Table I, application no. 19, has been published in A. And the activity of the gene product thereof is the activity of ycr102c-protein.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “ycr102c-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said Ycr102c, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Ycr102c, and preferably the activity is increased plastidic, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Ycr102c, or a functional equivalent or a homolog thereof as depicted in column 8 of Table II, application no. 19, preferably a homolog or functional equivalent as depicted in column 8 of Table II B, application no. 19, and being depicted in the same respective line as said Ycr102c, and preferably the activity is increased plastidic, whereby the respective line disclose in column 7 the fine chemical bis-glyceryl phosphate (polar fraction).

Accordingly, in one embodiment, the molecule which activity is to be increased in the process of the invention is the gene product with an activity as a “ycr102c-protein”, preferably it is the molecule of section (a) or (b) of this paragraph.

In particular, it was observed that in plants, especially in Arabidopsis thaliana, increasing or generating the activity of a gene product plastidic with the activity of a “ycr102c-protein”, preferably being encoded by a gene comprising the nucleic acid sequence SEQ ID NO. 63264, preferably the coding region thereof, conferred the production of or the increase in bis-glyceryl phosphate (polar fraction) compared with the wild type control.

The nucleic acid sequence of Ydr273w from Saccharomyces cerevisiae, e.g. as shown in the respective line in column 5 of Table I, application no. 19, has been published in A. And the activity of the gene product thereof is the activity of ydr273w-protein.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “ydr273w-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said Ydr273w, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Ydr273w, and preferably the activity is increased plastidic, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Ydr273w, or a functional equivalent or a homolog thereof as depicted in column 8 of Table II, application no. 19, preferably a homolog or functional equivalent as depicted in column 8 of Table II B, application no. 19, and being depicted in the same respective line as said Ydr273w, and preferably the activity is increased plastidic, whereby the respective line disclose in column 7 the fine chemical bis-glyceryl phosphate (polar fraction).

Accordingly, in one embodiment, the molecule which activity is to be increased in the process of the invention is the gene product with an activity as a “ydr273w-protein”, preferably it is the molecule of section (a) or (b) of this paragraph.

In particular, it was observed that in plants, especially in Arabidopsis thaliana, increasing or generating the activity of a gene product plastidic with the activity of a “ydr273w-protein”, preferably being encoded by a gene comprising the nucleic acid sequence SEQ ID NO. 90159, preferably the coding region thereof, conferred the production of or the increase in bis-glyceryl phosphate (polar fraction) compared with the wild type control.

The nucleic acid sequence of Yfl016c from Saccharomyces cerevisiae, e.g. as shown in the respective line in column 5 of Table I, application no. 19, has been published in A. And the activity of the gene product thereof is the activity of molecular chaperone portein.

Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “molecular chaperone portein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said Yfl016c, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Yfl016c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Yfl016c, or a functional equivalent or a homolog thereof as depicted in column 8 of Table II, application no. 19, preferably a homolog or functional equivalent as depicted in column 8 of Table II B, application no. 19, and being depicted in the same respective line as said Yfl016c, and preferably the activity is increased non-targeted, whereby the respective line disclose in column 7 the fine chemical glycerol phosphate (lipid fraction).

Accordingly, in one embodiment, the molecule which activity is to be increased in the process of the invention is the gene product with an activity as a “molecular chaperone portein”, preferably it is the molecule of section (a) or (b) of this paragraph.

In particular, it was observed that in plants, especially in Arabidopsis thaliana, increasing or generating the activity of a gene product non-targeted with the activity of a “molecular chaperone portein”, preferably being encoded by a gene comprising the nucleic acid sequence SEQ ID NO. 63807, preferably the coding region thereof, conferred the production of or the increase in glycerol phosphate (lipid fraction) compared with the wild type control.

The nucleic acid sequence of Yfl019c from Saccharomyces cerevisiae, e.g. as shown in the respective line in column 5 of Table I, application no. 19, has been published in A. And the activity of the gene product thereof is the activity of YFL019C-protein.

Accordingly, in one embodiment, the process of the present invention for producing triacylglycerol in a non-human organism, like a microorganism or a plant or a part thereof, comprises increasing or generating the activity of a gene product with the activity of a gene product conferring the activity of “YFL019C-protein”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical triacylglycerol), application no. 19, preferably the coding region thereof, or a homolog or a fragment thereof, and being depicted in the same respective line as said Yfl019c, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Yfl019c, and preferably the activity is increased non-targeted, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Yfl019c, or a functional equivalent or a homolog thereof as depicted in column 8 of Table II, application no. 19, preferably a homolog or functional equivalent as depicted in column 8 of Table 11 B, application no. 19, and being depicted in the same respective line as said Yfl019c, and preferably the activity is increased non-targeted, whereby the respective line disclose in column 7 the fine chemical triacylglycerol.

Accordingly, in one embodiment, the molecule which activity is to be increased in the process of the invention is the gene product with an activity as a “YFL0190-protein”, preferably it is the molecule of section (a) or (b) of this paragraph.

In particular, it was observed that in plants, especially in Arabidopsis thaliana, increasing or generating the activity of a gene product non-targeted with the activity of a “YFL019C-protein”, preferably being encoded by a gene comprising the nucleic acid sequence SEQ ID NO. 64144, preferably the coding region thereof, conferred the production of or the increase in triacylglycerol compared with the wild type control.

The nucleic acid sequence of Ygl256w from Saccharomyces cerevisiae, e.g. as shown in the respective line in column 5 of Table I, application no. 19, has been published in A. And the activity of the gene product thereof is the activity of alcohol dehydrogenase.

Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “alcohol dehydrogenase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said Ygl256w, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Ygl256w, and preferably the activity is increased plastidic, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Ygl256w, or a functional equivalent or a homolog thereof as depicted in column 8 of Table II, application no. 19, preferably a homolog or functional equivalent as depicted in column 8 of Table II B, application no. 19, and being depicted in the same respective line as said Ygl256w, and preferably the activity is increased plastidic, whereby the respective line disclose in column 7 the fine chemical bis-glyceryl phosphate (polar fraction).

Accordingly, in one embodiment, the molecule which activity is to be increased in the process of the invention is the gene product with an activity as a “alcohol dehydrogenase”, preferably it is the molecule of section (a) or (b) of this paragraph.

In particular, it was observed that in plants, especially in Arabidopsis thaliana, increasing or generating the activity of a gene product plastidic with the activity of a “alcohol dehydrogenase”, preferably being encoded by a gene comprising the nucleic acid sequence SEQ ID NO. 130742, preferably the coding region thereof, conferred the production of or the increase in bis-glyceryl phosphate (polar fraction) compared with the wild type control.

The nucleic acid sequence of Ygr286c from Saccharomyces cerevisiae, e.g. as shown in the respective line in column 5 of Table I, application no. 19, has been published in A. And the activity of the gene product thereof is the activity of biotin synthase.

Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “biotin synthase”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said Ygr286c, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Ygr286c, and preferably the activity is increased plastidic, or
(b) a polypeptide comprising a polypeptide, a consensus sequence or at least a polypeptide motif as shown in the respective line in column 5 of Table II or in column 8 of Table IV, application no. 19, respectively, and being depicted in the same respective line as said Ygr286c, or a functional equivalent or a homolog thereof as depicted in column 8 of Table II, application no. 19, preferably a homolog or functional equivalent as depicted in column 8 of Table II B, application no. 19, and being depicted in the same respective line as said Ygr286c, and preferably the activity is increased plastidic, whereby the respective line disclose in column 7 the fine chemical glycerol (lipid fraction).

Accordingly, in one embodiment, the molecule which activity is to be increased in the process of the invention is the gene product with an activity as a “biotin synthase”, preferably it is the molecule of section (a) or (b) of this paragraph.

In particular, it was observed that in plants, especially in Arabidopsis thaliana, increasing or generating the activity of a gene product plastidic with the activity of a “biotin synthase”, preferably being encoded by a gene comprising the nucleic acid sequence SEQ ID NO. 128655, preferably the coding region thereof, conferred the production of or the increase in glycerol (lipid fraction) compared with the wild type control.

The nucleic acid sequence of Yhr024c from Saccharomyces cerevisiae, e.g. as shown in the respective line in column 5 of Table I, application no. 19, has been published in A. And the activity of the gene product thereof is the activity of mitochondrial processing protease.

Accordingly, in one embodiment, the process of the present invention for producing triacylglycerol in a non-human organism, like a microorganism or a plant or a part thereof, comprises increasing or generating the activity of a gene product with the activity of a gene product conferring the activity of “mitochondrial processing protease”, especially from Saccharomyces cerevisiae or its functional equivalent or its homolog, e.g. the increase of
(a) a gene product of a gene comprising the nucleic acid molecule as shown in the respective line in column 5 of Table I (whereby the respective line disclose in column 7 the fine chemical triacylglycerol), application no. 19, preferably the coding region thereof, or a homolog or a fragment thereof, and being depicted in the same respective line as said Yhr024c, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Yhr024c, and preferably the activity is increased plastidic, or
[100000] (b) a polypeptide comprising a polypeptide, a consensus sequence or at least a polypeptide motif as shown in the respective line in column 5 of Table II or in column 8 of Table IV, application no. 19, respectively, and being depicted in the same respective line as said Yhr024c, or a functional equivalent or a homolog thereof as depicted in column 8 of Table II, application no. 19, preferably a homolog or functional equivalent as depicted in column 8 of Table II B, application no. 19, and being depicted in the same respective line as said Yhr024c, and preferably the activity is increased plastidic, whereby the respective line disclose in column 7 the fine chemical triacylglycerol.

[100001] Accordingly, in one embodiment, the molecule which activity is to be increased in the process of the invention is the gene product with an activity as a “mitochondrial processing protease”, preferably it is the molecule of section (a) or (b) of this paragraph.

[100002] In particular, it was observed that in plants, especially in Arabidopsis thaliana, increasing or generating the activity of a gene product plastidic with the activity of a “mitochondrial processing protease”, preferably being encoded by a gene comprising the nucleic acid sequence SEQ ID NO. 64336, preferably the coding region thereof, conferred the production of or the increase in triacylglycerol compared with the wild type control.

[100003] The nucleic acid sequence of Yhr202w from Saccharomyces cerevisiae, e.g. as shown in the respective line in column 5 of Table I, application no. 19, has been published in A. And the activity of the gene product thereof is the activity of YHR202W-protein.

[100004] Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “YHR202W-protein”, especially from Saccharomyces cerevisiae or its functional equivalent or its homolog, e.g. the increase of
[100005] (a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said Yhr202w, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Yhr202w, and preferably the activity is increased plastidic, or
[100006] (b) a polypeptide comprising a polypeptide, a consensus sequence or at least a polypeptide motif as shown in the respective line in column 5 of Table II or in column 8 of Table IV, application no. 19, respectively, and being depicted in the same respective line as said Yhr202w, or a functional equivalent or a homolog thereof as depicted in column 8 of Table II, application no. 19, preferably a homolog or functional equivalent as depicted in column 8 of Table II B, application no. 19, and being depicted in the same respective line as said Yhr202w, and preferably the activity is increased plastidic, whereby the respective line disclose in column 7 the fine chemical bis-glyceryl phosphate (polar fraction).

[100007] Accordingly, in one embodiment, the molecule which activity is to be increased in the process of the invention is the gene product with an activity as a “YHR202W-protein”, preferably it is the molecule of section (a) or (b) of this paragraph.

[100008] In particular, it was observed that in plants, especially in Arabidopsis thaliana, increasing or generating the activity of a gene product plastidic with the activity of a “YHR202W-protein”, preferably being encoded by a gene comprising the nucleic acid sequence SEQ ID NO. 154394, preferably the coding region thereof, conferred the production of or the increase in bis-glyceryl phosphate (polar fraction) compared with the wild type control.

[100009] The nucleic acid sequence of Yil053w from Saccharomyces cerevisiae, e.g. as shown in the respective line in column 5 of Table I, application no. 19, has been published in A. And the activity of the gene product thereof is the activity of (DL)-glycerol-3-phosphatase.

[100010] Accordingly, in one embodiment, the process of the present invention for producing glycerol (polar fraction) in a non-human organism, like a microorganism or a plant or a part thereof, comprises increasing or generating the activity of a gene product with the activity of a gene product conferring the activity of “(DL)-glycerol-3-phosphatase”, especially from Saccharomyces cerevisiae or its functional equivalent or its homolog, e.g. the increase of
[100011] (a) a gene product of a gene comprising the nucleic acid molecule as shown in the respective line in column 5 of Table I (whereby the respective line disclose in column 7 the fine chemical glycerol (polar fraction)), application no. 19, preferably the coding region thereof, or a homolog or a fragment thereof, and being depicted in the same respective line as said Yil053w, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Yil053w, and preferably the activity is increased non-targeted, or
[100012] (b) a polypeptide comprising a polypeptide, a consensus sequence or at least a polypeptide motif as shown in the respective line in column 5 of Table II or in column 8 of Table IV, application no. 19, respectively, and being depicted in the same respective line as said Yil053w, or a functional equivalent or a homolog thereof as depicted in column 8 of Table II, application no. 19, preferably a homolog or functional equivalent as depicted in column 8 of Table 11 B, application no. 19, and being depicted in the same respective line as said Yil053w, and preferably the activity is increased non-targeted, whereby the respective line disclose in column 7 the fine chemical glycerol (polar fraction).

[100013] Accordingly, in one embodiment, the molecule which activity is to be increased in the process of the invention is the gene product with an activity as a “(DL)-glycerol-3-phosphatase”, preferably it is the molecule of section (a) or (b) of this paragraph.

[100014] In particular, it was observed that in plants, especially in Arabidopsis thaliana, increasing or generating the activity of a gene product non-targeted with the activity of a “(DL)-glycerol-3-phosphatase”, preferably being encoded by a gene comprising the nucleic acid sequence SEQ ID NO. 14769, preferably the coding region thereof, conferred the production of or the increase in glycerol (polar fraction) compared with the wild type control.

[100015] The nucleic acid sequence of Yil083c from Saccharomyces cerevisiae, e.g. as shown in the respective line in column 5 of Table I, application no. 19, has been published in A. And the activity of the gene product thereof is the activity of YIL083C-protein.

[100016] Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “YIL083C-protein”, especially from Saccharomyces cerevisiae or its functional equivalent or its homolog, e.g. the increase of
[100017] (a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said Yil083c, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Yil083c, and preferably the activity is increased plastidic, or
[100018] (b) a polypeptide comprising a polypeptide, a consensus sequence or at least a polypeptide motif as shown in the respective line in column 5 of Table II or in column 8 of Table IV, application no. 19, respectively, and being depicted in the same respective line as said Yil083c, or a functional equivalent or a homolog thereof as depicted in column 8 of Table II, application no. 19, preferably a homolog or functional equivalent as depicted in column 8 of Table II B, application no. 19, and being depicted in the same respective line as said Yil083c, and preferably the activity is increased plastidic, whereby the respective line disclose in column 7 the fine chemical bis-glyceryl phosphate (polar fraction).

[100019] Accordingly, in one embodiment, the molecule which activity is to be increased in the process of the invention is the gene product with an activity as a “YIL083C-protein”, preferably it is the molecule of section (a) or (b) of this paragraph.

[100020] In particular, it was observed that in plants, especially in Arabidopsis thaliana, increasing or generating the activity of a gene product plastidic with the activity of a “YIL083C-protein”, preferably being encoded by a gene comprising the nucleic acid sequence SEQ ID NO. 149454, preferably the coding region thereof, conferred the production of or the increase in bis-glyceryl phosphate (polar fraction) compared with the wild type control.

[100021] The nucleic acid sequence of Yjl062w-a from Saccharomyces cerevisiae, e.g. as shown in the respective line in column 5 of Table I, application no. 19, has been published in A. And the activity of the gene product thereof is the activity of YJL062W-A-protein.

[100022] Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “YJL062W-A-protein”, especially from Saccharomyces cerevisiae or its functional equivalent or its homolog, e.g. the increase of
[100023] (a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said Yjl062w-a, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Yjl062w-a, and preferably the activity is increased non-targeted, or
[100024] (b) a polypeptide comprising a polypeptide, a consensus sequence or at least a polypeptide motif as shown in the respective line in column 5 of Table II or in column 8 of Table IV, application no. 19, respectively, and being depicted in the same respective line as said Yjl062w-a, or a functional equivalent or a homolog thereof as depicted in column 8 of Table II, application no. 19, preferably a homolog or functional equivalent as depicted in column 8 of Table II B, application no. 19, and being depicted in the same respective line as said Yjl062w-a, and preferably the activity is increased non-targeted, whereby the respective line disclose in column 7 the fine chemical glycerol phosphate (lipid fraction).

[100025] Accordingly, in one embodiment, the molecule which activity is to be increased in the process of the invention is the gene product with an activity as a “YJL062W-A-protein”, preferably it is the molecule of section (a) or (b) of this paragraph.

[100026] In particular, it was observed that in plants, especially in Arabidopsis thaliana, increasing or generating the activity of a gene product non-targeted with the activity of a “YJL062W-A-protein”, preferably being encoded by a gene comprising the nucleic acid sequence SEQ ID NO. 154437, preferably the coding region thereof, conferred the production of or the increase in glycerol phosphate (lipid fraction) compared with the wild type control.

[100027] The nucleic acid sequence of Yjl073w from Saccharomyces cerevisiae, e.g. as shown in the respective line in column 5 of Table I, application no. 19, has been published in A. And the activity of the gene product thereof is the activity of DnaJ-like chaperone.

[100028] Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “DnaJ-like chaperone”, especially from Saccharomyces cerevisiae or its functional equivalent or its homolog, e.g. the increase of
[100029] (a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said Yjl073w, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Yjl073w, and preferably the activity is increased plastidic, or
[100030] (b) a polypeptide comprising a polypeptide, a consensus sequence or at least a polypeptide motif as shown in the respective line in column 5 of Table II or in column 8 of Table IV, application no. 19, respectively, and being depicted in the same respective line as said Yjl073w, or a functional equivalent or a homolog thereof as depicted in column 8 of Table II, application no. 19, preferably a homolog or functional equivalent as depicted in column 8 of Table II B, application no. 19, and being depicted in the same respective line as said Yjl073w, and preferably the activity is increased plastidic, whereby the respective line disclose in column 7 the fine chemical bis-glyceryl phosphate (polar fraction).

[100031] Accordingly, in one embodiment, the molecule which activity is to be increased in the process of the invention is the gene product with an activity as a “DnaJ-like chaperone”, preferably it is the molecule of section (a) or (b) of this paragraph.

[100032] In particular, it was observed that in plants, especially in Arabidopsis thaliana, increasing or generating the activity of a gene product plastidic with the activity of a “DnaJ-like chaperone”, preferably being encoded by a gene comprising the nucleic acid sequence SEQ ID NO. 64964, preferably the coding region thereof, conferred the production of or the increase in bis-glyceryl phosphate (polar fraction) compared with the wild type control.

[100033] The nucleic acid sequence of Yjr153w from Saccharomyces cerevisiae, e.g. as shown in the respective line in column 5 of Table I, application no. 19, has been published in A. And the activity of the gene product thereof is the activity of polygalacturonase.

[100034] Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “polygalacturonase”, especially from Saccharomyces cerevisiae or its functional equivalent or its homolog, e.g. the increase of
[100035] (a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said Yjr153w, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Yjr153w, and preferably the activity is increased non-targeted, or
[100036] (b) a polypeptide comprising a polypeptide, a consensus sequence or at least a polypeptide motif as shown in the respective line in column 5 of Table II or in column 8 of Table IV, application no. 19, respectively, and being depicted in the same respective line as said Yjr153w, or a functional equivalent or a homolog thereof as depicted in column 8 of Table II, application no. 19, preferably a homolog or functional equivalent as depicted in column 8 of Table II B, application no. 19, and being depicted in the same respective line as said Yjr153w, and preferably the activity is increased non-targeted, whereby the respective line disclose in column 7 the fine chemical bis-glyceryl phosphate (polar fraction).

[100037] Accordingly, in one embodiment, the molecule which activity is to be increased in the process of the invention is the gene product with an activity as a “polygalacturonase”, preferably it is the molecule of section (a) or (b) of this paragraph.

[100038] In particular, it was observed that in plants, especially in Arabidopsis thaliana, increasing or generating the activity of a gene product non-targeted with the activity of a “polygalacturonase”, preferably being encoded by a gene comprising the nucleic acid sequence SEQ ID NO. 66274, preferably the coding region thereof, conferred the production of or the increase in bis-glyceryl phosphate (polar fraction) compared with the wild type control.

[100039] The nucleic acid sequence of Ylr025w from Saccharomyces cerevisiae, e.g. as shown in the respective line in column 5 of Table I, application no. 19, has been published in A. And the activity of the gene product thereof is the activity of vacuolar sorting protein.

[100040] Accordingly, in one embodiment, the process of the present invention for producing glycerol (polar fraction) in a non-human organism, like a microorganism or a plant or a part thereof, comprises increasing or generating the activity of a gene product with the activity of a gene product conferring the activity of “vacuolar sorting protein”, especially from Saccharomyces cerevisiae or its functional equivalent or its homolog, e.g. the increase of
[100041] (a) a gene product of a gene comprising the nucleic acid molecule as shown in the respective line in column 5 of Table I (whereby the respective line disclose in column 7 the fine chemical glycerol (polar fraction)), application no. 19, preferably the coding region thereof, or a homolog or a fragment thereof, and being depicted in the same respective line as said Ylr025w, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Ylr025w, and preferably the activity is increased non-targeted, or
[100042] (b) a polypeptide comprising a polypeptide, a consensus sequence or at least a polypeptide motif as shown in the respective line in column 5 of Table II or in column 8 of Table IV, application no. 19, respectively, and being depicted in the same respective line as said Ylr025w, or a functional equivalent or a homolog thereof as depicted in column 8 of Table II, application no. 19, preferably a homolog or functional equivalent as depicted in column 8 of Table II B, application no. 19, and being depicted in the same respective line as said Ylr025w, and preferably the activity is increased non-targeted, whereby the respective line disclose in column 7 the fine chemical glycerol (polar fraction).

[100043] Accordingly, in one embodiment, the molecule which activity is to be increased in the process of the invention is the gene product with an activity as a “vacuolar sorting protein”, preferably it is the molecule of section (a) or (b) of this paragraph.

[100044] In particular, it was observed that in plants, especially in Arabidopsis thaliana, increasing or generating the activity of a gene product non-targeted with the activity of a “vacuolar sorting protein”, preferably being encoded by a gene comprising the nucleic acid sequence SEQ ID NO. 150603, preferably the coding region thereof, conferred the production of or the increase in glycerol (polar fraction) compared with the wild type control.

[100045] The nucleic acid sequence of Ylr099c from Saccharomyces cerevisiae, e.g. as shown in the respective line in column 5 of Table I, application no. 19, has been published in A. And the activity of the gene product thereof is the activity of YLR099C-protein.

[100046] Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “YLR099C-protein”, especially from Saccharomyces cerevisiae or its functional equivalent or its homolog, e.g. the increase of
[100047] (a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said Ylr099c, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Ylr099c, and preferably the activity is increased plastidic, or
[100048] (b) a polypeptide comprising a polypeptide, a consensus sequence or at least a polypeptide motif as shown in the respective line in column 5 of Table II or in column 8 of Table IV, application no. 19, respectively, and being depicted in the same respective line as said Ylr099c, or a functional equivalent or a homolog thereof as depicted in column 8 of Table II, application no. 19, preferably a homolog or functional equivalent as depicted in column 8 of Table II B, application no. 19, and being depicted in the same respective line as said Ylr099c, and preferably the activity is increased plastidic, whereby the respective line disclose in column 7 the fine chemical bis-glyceryl phosphate (polar fraction).

[100049] Accordingly, in one embodiment, the molecule which activity is to be increased in the process of the invention is the gene product with an activity as a “YLR099C-protein”, preferably it is the molecule of section (a) or (b) of this paragraph.

[100050] In particular, it was observed that in plants, especially in Arabidopsis thaliana, increasing or generating the activity of a gene product plastidic with the activity of a “YLR099C-protein”, preferably being encoded by a gene comprising the nucleic acid sequence SEQ ID NO. 154454, preferably the coding region thereof, conferred the production of or the increase in bis-glyceryl phosphate (polar fraction) compared with the wild type control.

[100051] The nucleic acid sequence of Ylr210w from Saccharomyces cerevisiae, e.g. as shown in the respective line in column 5 of Table I, application no. 19, has been published in A. And the activity of the gene product thereof is the activity of cyclin.

[100052] Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “cyclin”, especially from Saccharomyces cerevisiae or its functional equivalent or its homolog, e.g. the increase of
[100053] (a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said Ylr210w, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Ylr210w, and preferably the activity is increased non-targeted, or
[100054] (b) a polypeptide comprising a polypeptide, a consensus sequence or at least a polypeptide motif as shown in the respective line in column 5 of Table II or in column 8 of Table IV, application no. 19, respectively, and being depicted in the same respective line as said Ylr210w, or a functional equivalent or a homolog thereof as depicted in column 8 of Table II, application no. 19, preferably a homolog or functional equivalent as depicted in column 8 of Table II B, application no. 19, and being depicted in the same respective line as said Ylr210w, and preferably the activity is increased non-targeted, whereby the respective line disclose in column 7 the fine chemical glycerol (lipid fraction).

[100055] Accordingly, in one embodiment, the molecule which activity is to be increased in the process of the invention is the gene product with an activity as a “cyclin”, preferably it is the molecule of section (a) or (b) of this paragraph.

[100056] In particular, it was observed that in plants, especially in Arabidopsis thaliana, increasing or generating the activity of a gene product non-targeted with the activity of a “cyclin”, preferably being encoded by a gene comprising the nucleic acid sequence SEQ ID NO. 129065, preferably the coding region thereof, conferred the production of or the increase in glycerol (lipid fraction) compared with the wild type control.

[100057] The nucleic acid sequence of Ylr326w from Saccharomyces cerevisiae, e.g. as shown in the respective line in column 5 of Table I, application no. 19, has been published in A. And the activity of the gene product thereof is the activity of ylr326w-protein.

[100058] Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “ylr326w-protein”, especially from Saccharomyces cerevisiae or its functional equivalent or its homolog, e.g. the increase of
[100059] (a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said Ylr326w, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Ylr326w, and preferably the activity is increased plastidic, or
[100060] (b) a polypeptide comprising a polypeptide, a consensus sequence or at least a polypeptide motif as shown in the respective line in column 5 of Table II or in column 8 of Table IV, application no. 19, respectively, and being depicted in the same respective line as said Ylr326w, or a functional equivalent or a homolog thereof as depicted in column 8 of Table II, application no. 19, preferably a homolog or functional equivalent as depicted in column 8 of Table II B, application no. 19, and being depicted in the same respective line as said Ylr326w, and preferably the activity is increased plastidic, whereby the respective line disclose in column 7 the fine chemical glycerol (lipid fraction).

[100061] Accordingly, in one embodiment, the molecule which activity is to be increased in the process of the invention is the gene product with an activity as a “ylr326w-protein”, preferably it is the molecule of section (a) or (b) of this paragraph.

[100062] In particular, it was observed that in plants, especially in Arabidopsis thaliana, increasing or generating the activity of a gene product plastidic with the activity of a “ylr326w-protein”, preferably being encoded by a gene comprising the nucleic acid sequence SEQ ID NO. 129123, preferably the coding region thereof, conferred the production of or the increase in glycerol (lipid fraction) compared with the wild type control.

[100063] The nucleic acid sequence of Ylr326w from Saccharomyces cerevisiae, e.g. as shown in the respective line in column 5 of Table I, application no. 19, has been published in A. And the activity of the gene product thereof is the activity of ylr326w-protein.

[100064] Accordingly, in one embodiment, the process of the present invention for producing glycerol phosphate (lipid fraction) in a non-human organism, like a microorganism or a plant or a part thereof, comprises increasing or generating the activity of a gene product with the activity of a gene product conferring the activity of “ylr326w-protein”, especially from Saccharomyces cerevisiae or its functional equivalent or its homolog, e.g. the increase of
[100065] (a) a gene product of a gene comprising the nucleic acid molecule as shown in the respective line in column 5 of Table I (whereby the respective line disclose in column 7 the fine chemical glycerol phosphate (lipid fraction)), application no. 19, preferably the coding region thereof, or a homolog or a fragment thereof, and being depicted in the same respective line as said Ylr326w, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Ylr326w, and preferably the activity is increased plastidic, or
[100066] (b) a polypeptide comprising a polypeptide, a consensus sequence or at least a polypeptide motif as shown in the respective line in column 5 of Table II or in column 8 of Table IV, application no. 19, respectively, and being depicted in the same respective line as said Ylr326w, or a functional equivalent or a homolog thereof as depicted in column 8 of Table II, application no. 19, preferably a homolog or functional equivalent as depicted in column 8 of Table II B, application no. 19, and being depicted in the same respective line as said Ylr326w, and preferably the activity is increased plastidic, whereby the respective line disclose in column 7 the fine chemical glycerol phosphate (lipid fraction).

[100067] Accordingly, in one embodiment, the molecule which activity is to be increased in the process of the invention is the gene product with an activity as a “ylr326w-protein”, preferably it is the molecule of section (a) or (b) of this paragraph.

[100068] In particular, it was observed that in plants, especially in Arabidopsis thaliana, increasing or generating the activity of a gene product plastidic with the activity of a “ylr326w-protein”, preferably being encoded by a gene comprising the nucleic acid sequence SEQ ID NO. 129123, preferably the coding region thereof, conferred the production of or the increase in glycerol phosphate (lipid fraction) compared with the wild type control.

[100069] The nucleic acid sequence of Ynl007c from Saccharomyces cerevisiae, e.g. as shown in the respective line in column 5 of Table I, application no. 19, has been published in A. And the activity of the gene product thereof is the activity of heat shock protein Hsp40.

[100070] Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “heat shock protein Hsp40”, especially from Saccharomyces cerevisiae or its functional equivalent or its homolog, e.g. the increase of
[100071] (a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said Ynl007c, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Ynl007c, and preferably the activity is increased non-targeted, or
[100072] (b) a polypeptide comprising a polypeptide, a consensus sequence or at least a polypeptide motif as shown in the respective line in column 5 of Table II or in column 8 of Table IV, application no. 19, respectively, and being depicted in the same respective line as said Ynl007c, or a functional equivalent or a homolog thereof as depicted in column 8 of Table II, application no. 19, preferably a homolog or functional equivalent as depicted in column 8 of Table II B, application no. 19, and being depicted in the same respective line as said Ynl007c, and preferably the activity is increased non-targeted, whereby the respective line disclose in column 7 the fine chemical glycerol (lipid fraction).

[100073] Accordingly, in one embodiment, the molecule which activity is to be increased in the process of the invention is the gene product with an activity as a “heat shock protein Hsp40”, preferably it is the molecule of section (a) or (b) of this paragraph.

[100074] In particular, it was observed that in plants, especially in Arabidopsis thaliana, increasing or generating the activity of a gene product non-targeted with the activity of a “heat shock protein Hsp40”, preferably being encoded by a gene comprising the nucleic acid sequence SEQ ID NO. 139173, preferably the coding region thereof, conferred the production of or the increase in glycerol (lipid fraction) compared with the wild type control.

[100075] The nucleic acid sequence of Ynl130c from Saccharomyces cerevisiae, e.g. as shown in the respective line in column 5 of Table I, application no. 19, has been published in A. And the activity of the gene product thereof is the activity of phosphotransferase.

[100076] Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “phosphotransferase”, especially from Saccharomyces cerevisiae or its functional equivalent or its homolog, e.g. the increase of
[100077] (a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said Ynl130c, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Ynl130c, and preferably the activity is increased non-targeted, or
[100078] (b) a polypeptide comprising a polypeptide, a consensus sequence or at least a polypeptide motif as shown in the respective line in column 5 of Table II or in column 8 of Table IV, application no. 19, respectively, and being depicted in the same respective line as said Ynl130c, or a functional equivalent or a homolog thereof as depicted in column 8 of Table II, application no. 19, preferably a homolog or functional equivalent as depicted in column 8 of Table II B, application no. 19, and being depicted in the same respective line as said Ynl130c, and preferably the activity is increased non-targeted, whereby the respective line disclose in column 7 the fine chemical glycerol (lipid fraction).

[100079] Accordingly, in one embodiment, the molecule which activity is to be increased in the process of the invention is the gene product with an activity as a “phosphotransferase”, preferably it is the molecule of section (a) or (b) of this paragraph.

[100080] In particular, it was observed that in plants, especially in Arabidopsis thaliana, increasing or generating the activity of a gene product non-targeted with the activity of a “phosphotransferase”, preferably being encoded by a gene comprising the nucleic acid sequence SEQ ID NO. 129470, preferably the coding region thereof, conferred the production of or the increase in glycerol (lipid fraction) compared with the wild type control.

[100081] The nucleic acid sequence of Yor187w from Saccharomyces cerevisiae, e.g. as shown in the respective line in column 5 of Table I, application no. 19, has been published in A. And the activity of the gene product thereof is the activity of Elongation factor Tu.

[100082] Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “Elongation factor Tu”, especially from Saccharomyces cerevisiae or its functional equivalent or its homolog, e.g. the increase of
[100083] (a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said Yor187w, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Yor187w, and preferably the activity is increased non-targeted, or
[100084] (b) a polypeptide comprising a polypeptide, a consensus sequence or at least a polypeptide motif as shown in the respective line in column 5 of Table II or in column 8 of Table IV, application no. 19, respectively, and being depicted in the same respective line as said Yor187w, or a functional equivalent or a homolog thereof as depicted in column 8 of Table II, application no. 19, preferably a homolog or functional equivalent as depicted in column 8 of Table II B, application no. 19, and being depicted in the same respective line as said Yor187w, and preferably the activity is increased non-targeted, whereby the respective line disclose in column 7 the fine chemical glycerol (lipid fraction).

[100085] Accordingly, in one embodiment, the molecule which activity is to be increased in the process of the invention is the gene product with an activity as a “Elongation factor Tu”, preferably it is the molecule of section (a) or (b) of this paragraph.

[100086] In particular, it was observed that in plants, especially in Arabidopsis thaliana, increasing or generating the activity of a gene product non-targeted with the activity of a “Elongation factor Tu”, preferably being encoded by a gene comprising the nucleic acid sequence SEQ ID NO. 129574, preferably the coding region thereof, conferred the production of or the increase in glycerol (lipid fraction) compared with the wild type control.

[100087] The nucleic acid sequence of Ypl162c from Saccharomyces cerevisiae, e.g. as shown in the respective line in column 5 of Table I, application no. 19, has been published in A. And the activity of the gene product thereof is the activity of YPL162C.protein.

[100088] Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “YPL162C.protein”, especially from Saccharomyces cerevisiae or its functional equivalent or its homolog, e.g. the increase of
[100089] (a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said Ypl162c, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Ypl162c, and preferably the activity is increased plastidic, or
[100090] (b) a polypeptide comprising a polypeptide, a consensus sequence or at least a polypeptide motif as shown in the respective line in column 5 of Table II or in column 8 of Table IV, application no. 19, respectively, and being depicted in the same respective line as said Ypl162c, or a functional equivalent or a homolog thereof as depicted in column 8 of Table II, application no. 19, preferably a homolog or functional equivalent as depicted in column 8 of Table II B, application no. 19, and being depicted in the same respective line as said Ypl162c, and preferably the activity is increased plastidic, whereby the respective line disclose in column 7 the fine chemical bis-glyceryl phosphate (polar fraction).

[100091] Accordingly, in one embodiment, the molecule which activity is to be increased in the process of the invention is the gene product with an activity as a “YPL162C.protein”, preferably it is the molecule of section (a) or (b) of this paragraph.

[100092] In particular, it was observed that in plants, especially in Arabidopsis thaliana, increasing or generating the activity of a gene product plastidic with the activity of a “YPL162C.protein”, preferably being encoded by a gene comprising the nucleic acid sequence SEQ ID NO. 154477, preferably the coding region thereof, conferred the production of or the increase in bis-glyceryl phosphate (polar fraction) compared with the wild type control.

[100093] The nucleic acid sequence of Ypr054w from Saccharomyces cerevisiae, e.g. as shown in the respective line in column 5 of Table I, application no. 19, has been published in A. And the activity of the gene product thereof is the activity of mitogen-activated protein kinase.

[100094] Accordingly, in one embodiment, the process of the present invention for producing glycerol (lipid fraction) in a non-human organism, like a microorganism or a plant or a part thereof, comprises increasing or generating the activity of a gene product with the activity of a gene product conferring the activity of “mitogen-activated protein kinase”, especially from Saccharomyces cerevisiae or its functional equivalent or its homolog, e.g. the increase of
[100095] (a) a gene product of a gene comprising the nucleic acid molecule as shown in the respective line in column 5 of Table I (whereby the respective line disclose in column 7 the fine chemical glycerol (lipid fraction)), application no. 19, preferably the coding region thereof, or a homolog or a fragment thereof, and being depicted in the same respective line as said Ypr054w, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Ypr054w, and preferably the activity is increased non-targeted, or
[100096] (b) a polypeptide comprising a polypeptide, a consensus sequence or at least a polypeptide motif as shown in the respective line in column 5 of Table II or in column 8 of Table IV, application no. 19, respectively, and being depicted in the same respective line as said Ypr054w, or a functional equivalent or a homolog thereof as depicted in column 8 of Table II, application no. 19, preferably a homolog or functional equivalent as depicted in column 8 of Table II B, application no. 19, and being depicted in the same respective line as said Ypr054w, and preferably the activity is increased non-targeted, whereby the respective line disclose in column 7 the fine chemical glycerol (lipid fraction).

[100097] Accordingly, in one embodiment, the molecule which activity is to be increased in the process of the invention is the gene product with an activity as a “mitogen-activated protein kinase”, preferably it is the molecule of section (a) or (b) of this paragraph.

[100098] In particular, it was observed that in plants, especially in Arabidopsis thaliana, increasing or generating the activity of a gene product non-targeted with the activity of a “mitogen-activated protein kinase”, preferably being encoded by a gene comprising the nucleic acid sequence SEQ ID NO. 140110, preferably the coding region thereof, conferred the production of or the increase in glycerol (lipid fraction) compared with the wild type control.

[100099] The nucleic acid sequence of Zm_{—}4842_BE510522 from Zea mays, e.g. as shown in the respective line in column 5 of Table I, application no. 19, is unpublished. And the activity of the gene product thereof is the activity of Zm_{—}4842_BE510522-protein.

[100100] Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “Zm_{—}4842_BE510522-protein”, especially from Zea mays or its functional equivalent or its homolog, e.g. the increase of
[100101] (a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said Zm_{—}4842_BE510522, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said Zm_{—}4842_BE510522, and preferably the activity is increased non-targeted, or
[100102] (b) a polypeptide comprising a polypeptide, a consensus sequence or at least a polypeptide motif as shown in the respective line in column 5 of Table II or in column 8 of Table IV, application no. 19, respectively, and being depicted in the same respective line as said Zm_{—}4842_BE510522, or a functional equivalent or a homolog thereof as depicted in column 8 of Table II, application no. 19, preferably a homolog or functional equivalent as depicted in column 8 of Table II B, application no. 19, and being depicted in the same respective line as said Zm_{—}4842_BE510522, and preferably the activity is increased non-targeted, whereby the respective line disclose in column 7 the fine chemical bis-glyceryl phosphate (polar fraction).

[100103] Accordingly, in one embodiment, the molecule which activity is to be increased in the process of the invention is the gene product with an activity as a “Zm_{—}4842_BE510522-protein”, preferably it is the molecule of section (a) or (b) of this paragraph.

[100104] In particular, it was observed that in plants, especially in Arabidopsis thaliana, increasing or generating the activity of a gene product non-targeted with the activity of a “Zm_{—}4842_BE510522-protein”, preferably being encoded by a gene comprising the nucleic acid sequence SEQ ID NO. 68413, preferably the coding region thereof, conferred the production of or the increase in bis-glyceryl phosphate (polar fraction) compared with the wild type control.

[100105] The nucleic acid sequence of 49747384_SOYBEAN from Glycine max, e.g. as shown in the respective line in column 5 of Table I, application no. 19, is unpublished. And the activity of the gene product thereof is the activity of 49747384_SOYBEAN-protein.

[100106] Accordingly, in one embodiment, the process of the present invention for producing bis-glyceryl phosphate (polar fraction) in a non-human organism, like a microorganism or a plant or a part thereof, comprises increasing or generating the activity of a gene product with the activity of a gene product conferring the activity of “49747384_SOYBEAN-protein”, especially from Glycine max or its functional equivalent or its homolog, e.g. the increase of
[100107] (a) a gene product of a gene comprising the nucleic acid molecule as shown in the respective line in column 5 of Table I (whereby the respective line disclose in column 7 the fine chemical bis-glyceryl phosphate (polar fraction)), application no. 19, preferably the coding region thereof, or a homolog or a fragment thereof, and being depicted in the same respective line as said 49747384_SOYBEAN, or a functional equivalent or a homolog thereof as shown in column 8 of Table I, application no. 19, preferably the coding region thereof, particularly a homolog or functional equivalent as shown in column 8 of Table I B, application no. 19, and being depicted in the same respective line as said 49747384_SOYBEAN, and preferably the activity is increased non-targeted, or
[100108] (b) a polypeptide comprising a polypeptide, a consensus sequence or at least a polypeptide motif as shown in the respective line in column 5 of Table II or in column 8 of Table IV, application no. 19, respectively, and being depicted in the same respective line as said 49747384_SOYBEAN, or a functional equivalent or a homolog thereof as depicted in column 8 of Table II, application no. 19, preferably a homolog or functional equivalent as depicted in column 8 of Table II B, application no. 19, and being depicted in the same respective line as said 49747384_SOYBEAN, and preferably the activity is increased non-targeted, whereby the respective line disclose in column 7 the fine chemical bis-glyceryl phosphate (polar fraction).

[100109] Accordingly, in one embodiment, the molecule which activity is to be increased in the process of the invention is the gene product with an activity as a “49747384_SOYBEAN-protein”, preferably it is the molecule of section (a) or (b) of this paragraph.

[100110] In particular, it was observed that in plants, especially in Arabidopsis thaliana, increasing or generating the activity of a gene product non-targeted with the activity of a “49747384_SOYBEAN-protein”, preferably being encoded by a gene comprising the nucleic acid sequence SEQ ID NO. 69, preferably the coding region thereof, conferred the production of or the increase in bis-glyceryl phosphate (polar fraction) compared with the wild type control.

[100111] [0105.1.7.19] to [0107.1.7.19] for the disclosure of these paragraphs see [0105.1.7.7] to [0107.1.7.7] above

[100112] A protein having an activity conferring an increase in the amount or level of the fine chemical bis-glyceryl phosphate (polar fraction), glycerol (lipid fraction), glycerol (polar fraction), glycerol phosphate (lipid fraction), glycerol-3-phosphate (polar fraction), or triacylglycerol, upon targeting to the plastids or mitochondria or upon non-targeting, preferably has the structure of the respective polypeptide described herein, in particular of the polypeptides comprising the consensus sequence or at least one polypeptide motifs as shown in the respective line in Table IV, application no. 19, column 8 or of the polypeptide comprising an amino acid sequence as disclosed in the respective line in Table II, application no. 19, columns 5 or 8, or homologs or fragments thereof as described herein, or is encoded by the nucleic acid molecule characterized herein or the nucleic acid molecule according to the invention, for example by the nucleic acid molecule as shown in the respective line in Table I, application no. 19, columns 5 or 8, preferably the coding region thereof, or homologs or fragments thereof and has the herein mentioned activity.

[100113] for the disclosure of this paragraph see [0109.1.7.7] above

[100114] for the disclosure of this paragraph see [0110.1.7.7] above

[100115] In one embodiment, the process of the present invention comprises one or more of the following steps
[100116] (a) stabilizing a protein conferring the generation or increased expression of a protein encoded by the nucleic acid molecule of the invention or of the polypeptide of the invention, e.g. of a polypeptide having the activity selected from the group consisting of (DL)-glycerol-3-phosphatase, 2-C-methyl-D-erythritol 4-phosphate cytidylyltransferase, 3-oxoacyl-[acyl-carrier-protein] synthase, 49747384_SOYBEAN-protein, ABC transporter permease protein, acetyl CoA carboxylase, acid phosphatase precursor, acid shock protein, acyl transferase, adenylate kinase, alcohol dehydrogenase, amino-acid acetyltransferase, arginine decarboxylase, argininosuccinate synthetase, aromatic acid decarboxylase, At1g23110-protein, At1g67340-protein, ATP-binding component of a transport system, ATP-dependent RNA helicase, b0050-protein, b0518-protein, b0970-protein, b1003-protein, b1234-protein, b1330-protein, b1522-protein, b1556-protein, b1583-protein, b1670-protein, b1672-protein, b1703-protein, B1795-protein, b1837-protein, b2032-protein, b2099-protein, b2139-protein, b2399-protein, b2474-protein, b2613-protein, b2673-protein, b3083-protein, b3160-protein, b3777-protein, b3791-protein, b3989-protein, b4029-protein, b4050-protein, beta-hydroxylase, biotin synthase, calcium-dependent protein kinase, carbamoyl-phosphate synthase subunit, carbohydrate kinase, cardiolipin synthase, CBL-interacting protein kinase, CDP-diacylglycerol-glycerol-3-phosphate 3-phosphatidyltransferase, cell division control protein, cell division protein, coproporphyrinogen III oxidase, cyclin, cyclin D , dihydrolipoamide dehydrogenase, DnaJ-like chaperone, Elongation factor Tu, enterobacterial common antigen polymerase, ethanolamine utilization protein, eukaryotic translation initiation factor 5, Fe—S subunit of oxidoreductase, FKBP-type peptidyl-prolyl cis-trans isomerase (rotamase), glutamate 5-kinase, glutaredoxin, Glycine cleavage system T aminomethyltransferase, GM02LC17485-protein, heat shock protein Hsp40, heat shock transcription factor, HesB/YadR/YfhF family protein, high-affinity branched-chain amino acid transport protein, hydrolase, imidazole glycerol phosphate synthase subunit, ion-transport protein, lipoprotein precursor, L-serine dehydratase, lysozyme, lysyl-tRNA synthetase, metal-dependent hydrolase, methylglyoxal synthase, methyltransferase, Mg-protoporphyrin IX monomethyl ester oxidative cyclase 66 kD subunit, mitochondrial processing protease, mitogen-activated protein kinase, molecular chaperone portein, monothiol glutaredoxin, murein transglycosylase, NADH dehydrogenase, NADH-quinone oxidoreductase subunit, nitrate/nitrite transport protein, oxidoreductase, phenylacetic acid degradation protein, phosphate import ATP-binding protein, phosphatidylinositol 3- and 4-kinase family protein, phosphoglyceromutase, phosphotransferase, phosphotransferase system component, photosystem I reaction centre subunit, polygalacturonase, precorrin-8X methylmutase, protein phosphatase, purine-nucleoside phosphorylase, putative transport system permease protein, putrescine transport system permease protein, pyruvate kinase, recombinase A, sec-independent protein translocase, serine protease, sodium/proton antiporter, ThiF family protein, threonine dehydratase, threonine synthase, threonine/serine transporter, transcription factor, transcriptional regulator, transcriptional regulafor protein, trehalose-phosphatase, TTC1386-protein, vacuolar sorting protein, YAL013W-protein, ycr102c-protein, ydr273w-protein, YFL019C-protein, YHR202W-protein, YIL083C-protein, YJL062W-A-protein, YLR099C-protein, ylr326w-protein, YPL162C.protein, and Zm_{—}4842_BE510522-protein, or of a polypeptide as indicated in the respective line in Table II, application no. 19, columns 5 or 8, or its homologs or fragments, and conferring the production of or an increase in bis-glyceryl phosphate (polar fraction), glycerol (lipid fraction), glycerol (polar fraction), glycerol phosphate (lipid fraction), glycerol-3-phosphate (polar fraction), or triacylglycerol, respectively; and/or
[100117] (b) stabilizing a mRNA conferring the generation or increased expression of a FCRP, e.g. protein encoded by the nucleic acid molecule of the invention or its homologs or fragments, or of a mRNA encoding the polypeptide of the present invention having the hereinmentioned activity selected from the group consisting of said activities mentioned in (a) and conferring the production of or an increase in bis-glyceryl phosphate (polar fraction), glycerol (lipid fraction), glycerol (polar fraction), glycerol phosphate (lipid fraction), glycerol-3-phosphate (polar fraction), or triacylglycerol, respectively; and/or
[100118] (c) increasing the specific activity of a protein conferring the increased expression of a FCRP, e.g. a protein encoded by the nucleic acid molecule of the invention or of the polypeptide of the present invention having herein-mentioned bis-glyceryl phosphate (polar fraction), glycerol (lipid fraction), glycerol (polar fraction), glycerol phosphate (lipid fraction), glycerol-3-phosphate (polar fraction), or triacylglycerol generating or increasing activity, respectively, e.g. of a polypeptide having the activity of a protein as indicated in the respective line in Table II, application no. 19, columns 5 or 8, or its homologs or fragments, or decreasing the inhibitory regulation of the polypeptide of the invention; and/or
[100119] (d) generating or increasing the expression of an endogenous or artificial transcription factor mediating the expression of a protein conferring the generation or increased expression of a FCRP, e.g. a protein encoded by the nucleic acid molecule of the invention or of the polypeptide of the invention having herein-mentioned activity selected from the group consisting of said activities mentioned in (a) and conferring a bis-glyceryl phosphate (polar fraction), glycerol (lipid fraction), glycerol (polar fraction), glycerol phosphate (lipid fraction), glycerol-3-phosphate (polar fraction), or triacylglycerol increasing activity, respectively, e.g. of a polypeptide having the activity of a protein as indicated in the respective line in Table II, application no. 19, columns 5 or 8 or its homologs; and/or
[100120] (e) stimulating activity of a protein conferring the increased expression of a FCRP, e.g. a protein encoded by the nucleic acid molecule of the present invention or a polypeptide of the present invention having herein-mentioned activity selected from the group consisting of said activities mentioned in (a) and conferring a bis-glyceryl phosphate (polar fraction), glycerol (lipid fraction), glycerol (polar fraction), glycerol phosphate (lipid fraction), glycerol-3-phosphate (polar fraction), or triacylglycerol increasing activity, respectively, e.g. of a polypeptide having the activity of a protein as indicated in the respective line in Table II, application no. 19, columns 5 or 8, or its homologs or fragments, by adding one or more exogenous inducing factors to the non-human organism or parts thereof; and/or
[100121] (f) expressing a transgenic gene encoding a protein conferring the increased expression of a FCRP, e.g. a polypeptide encoded by the nucleic acid molecule of the present invention or a polypeptide of the present invention, having herein-mentioned activity selected from the group consisting of said activities mentioned in (a) and conferring a bis-glyceryl phosphate (polar fraction), glycerol (lipid fraction), glycerol (polar fraction), glycerol phosphate (lipid fraction), glycerol-3-phosphate (polar fraction), or triacylglycerol increasing activity, respectively, e.g. of a polypeptide having the activity of a protein as indicated in the respective line in Table II, application no. 19, columns 5 or 8, or its homologs or fragments, and/or
[100122] (g) increasing the copy number of a gene conferring the increased expression of a nucleic acid molecule encoding a FCRP, e.g. a polypeptide encoded by the nucleic acid molecule of the invention or the polypeptide of the invention having herein-mentioned activity selected from the group consisting of said activities mentioned in (a) and conferring a bis-glyceryl phosphate (polar fraction), glycerol (lipid fraction), glycerol (polar fraction), glycerol phosphate (lipid fraction), glycerol-3-phosphate (polar fraction), or triacylglycerol increasing activity, respectively, e.g. of a polypeptide having the activity of a protein as indicated in the respective line in Table II, application no. 19, columns 5 or 8, or its homologs or fragments; and/or
[100123] (h) increasing the expression of the endogenous gene encoding the FCRP, polypeptide of the invention, e.g. a polypeptide encoded by the nucleic acid molecule of the invention or the polypeptide of the invention having herein-mentioned activity selected from the group consisting of said activities mentioned in (a) and conferring a bis-glyceryl phosphate (polar fraction), glycerol (lipid fraction), glycerol (polar fraction), glycerol phosphate (lipid fraction), glycerol-3-phosphate (polar fraction), or triacylglycerol; increasing activity, respectively, e.g. of a polypeptide having the activity of a protein as indicated in the respective line in Table II, application no. 19, columns 5 or 8, or its homologs or fragments, by adding positive expression or removing negative expression elements, e.g. homologous recombination can be used to either introduce positive regulatory elements like for plants the 35S enhancer into the promoter or to remove repressor elements form regulatory regions. Further gene conversion methods can be used to disrupt repressor elements or to enhance to activity of positive elements. Positive elements can be randomly introduced in plants by T-DNA or transposon mutagenesis and lines can be identified in which the positive elements have be integrated near to a gene of the invention, the expression of which is thereby enhanced; and/or
[100124] (i) modulating growth conditions of the non-human organism in such a manner, that the expression or activity of the gene encoding the FCRP, e.g. a protein of the invention or the protein itself is enhanced, for example microorganisms or plants can be grown for example under a higher temperature regime leading to an enhanced expression of heat shock proteins, which can lead to an enhanced bis-glyceryl phosphate (polar fraction), glycerol (lipid fraction), glycerol (polar fraction), glycerol phosphate (lipid fraction), glycerol-3-phosphate (polar fraction), or triacylglycerol production, respectively; and/or
[100125] (j) selecting of non-human organisms with especially high activity of the proteins of the invention from natural or from mutagenized resources and breeding them into the target organisms, e.g. the elite crops; and/or
[100126] (k) directing a protein encoded by the nucleic acid molecule of the invention or of the polypeptide of the present invention having herein-mentioned activity selected from the group consisting of said activities mentioned in (a) and conferring a bis-glyceryl phosphate (polar fraction), glycerol (lipid fraction), glycerol (polar fraction), glycerol phosphate (lipid fraction), glycerol-3-phosphate (polar fraction), or triacylglycerol increasing activity, respectively, e.g. of a polypeptide having the activity of a protein as indicated in the respective line in Table II, application no. 19, columns 5 or 8, or its homologs or fragments, if for the corresponding nucleic acid molecule in the respective line in column 6 of Table I the term “plastidic” is indicated, to the plastids by the addition of a plastidial targeting sequence or if for the corresponding nucleic acid molecule in the respective line in column 6 of Table I the term “mitochondric” is indicated, to the mitochondria by the addition of a mitochondrial targeting sequence; and/or
[100127] (l) generating the expression of a protein encoded by the nucleic acid molecule of the invention or of the polypeptide of the present invention having herein-mentioned activity selected from the group consisting of said activities mentioned in (a) and conferring a bis-glyceryl phosphate (polar fraction), glycerol (lipid fraction), glycerol (polar fraction), glycerol phosphate (lipid fraction), glycerol-3-phosphate (polar fraction), or triacylglycerol increasing activity, respectively, e.g. of a polypeptide having the activity of a protein as indicated in the respective line in Table II, application no. 19, columns 5 or 8, or its homologs or fragments, if for the corresponding nucleic acid molecule in the respective line in column 6 of Table I the term “plastidic” or “mitochondric” is indicated, in these organelles by the stable or transient transformation, advantageously stable transformation, of organelles, preferably plastids or mitochondria, with an inventive nucleic acid sequence preferably in form of an expression cassette containing said sequence leading to the expression of the nucleic acids or polypeptides of the invention in the respective organelle; and/or
[100128] (m) generating the expression of a protein encoded by the nucleic acid molecule of the invention or of the polypeptide of the present invention having herein-mentioned activity selected from the group consisting of said activities mentioned in (a) and conferring a bis-glyceryl phosphate (polar fraction), glycerol (lipid fraction), glycerol (polar fraction), glycerol phosphate (lipid fraction), glycerol-3-phosphate (polar fraction), and/or triacylglycerol increasing activity, respectively, e.g. of a polypeptide having the activity of a protein as indicated in the respective line in Table II, application no. 19, columns 5 or 8, or its homologs or fragments, if for the corresponding nucleic acid molecule in the respective line in column 6 of Table I the term “plastidic” or “mitochondric” is indicated, in these organelles by integration of a nucleic acid of the invention into the genome of the respective organelle under control of preferable a promoter selective for the respective organelle.

[100129] Preferably, said mRNA is the coding region of the nucleic acid molecule of the present invention and/or the protein conferring the increased expression of a protein encoded by the coding region of the nucleic acid molecule of the present invention alone or linked to a transit nucleic acid sequence or transit peptide encoding nucleic acid sequence or the polypeptide having the herein mentioned activity, e.g. conferring the generation of or increase of bis-glyceryl phosphate (polar fraction), glycerol (lipid fraction), glycerol (polar fraction), glycerol phosphate (lipid fraction), glycerol-3-phosphate (polar fraction), or triacylglycerol, respectively, after increasing the expression or activity of the encoded polypeptide, non-targeted or in organelles such as plastids and/or mitochondria, preferably plastids, or having the activity of a polypeptide having an activity as the protein as shown in the respective line in Table II, application no. 19, column 3, or its homologs. Preferably the increase of bis-glyceryl phosphate (polar fraction), glycerol (lipid fraction), glycerol (polar fraction), glycerol phosphate (lipid fraction), glycerol-3-phosphate (polar fraction), or triacylglycerol, respectively, takes place non-targeted or in plastids and/or mitochondria, preferably non-targeted or in plastids.

[100130] [0113.1.7.19] to [0122.1.7.19] for the disclosure of these paragraphs see [0113.1.7.7] to [0122.1.7.7] above

[100131] The activation of an endogenous polypeptide having above-mentioned activity, e.g. having the activity of a protein as shown in the respective line in Table II, application no. 19, column 3 or of the polypeptide of the invention, e.g. conferring the increase of the fine chemical bis-glyceryl phosphate (polar fraction), glycerol (lipid fraction), glycerol (polar fraction), glycerol phosphate (lipid fraction), glycerol-3-phosphate (polar fraction), or triacylglycerol, respectively, by increase of expression or activity in the cytoplasm, and/or in the cytosol, and/or in an organelle, such as plastids or mitochondria, can also be increased by introducing a synthetic transcription factor, which binds close to the coding region of the gene encoding the protein as shown in the respective line in Table II, application no. 19, column 5 or 8, or homologs or fragments thereof, and activates its transcription. A chimeric zinc finger protein can be constructed, which comprises a specific DNA-binding domain and an activation domain as e.g. the VP16 domain of Herpes Simplex virus. The specific binding domain can bind to the regulatory region of the gene encoding the protein as shown in the respective line in Table II, application no. 19, column 5 or 8, or homologs or fragments thereof. The expression of the chimeric transcription factor in a non-human organism, in particular in a plant, leads to a specific expression of the protein as shown in the respective line in Table II, application no. 19, column 5 or 8, or homologs or fragments thereof, see e.g. in WO01/52620, Oriz, Proc. Natl. Acad. Sci. USA 99, 13290 (2002), or Guan, Proc. Natl. Acad. Sci. USA 99, 13296 (2002).

[100132] [0124.1.7.19] to [0127.1.7.19] for the disclosure of these paragraphs see [0124.1.7.7] to [0127.1.7.7] above

[100133] Owing to the introduction of a gene or a plurality of genes conferring the expression of the nucleic acid molecule of the invention or the polypeptide of the invention, for example the nucleic acid construct mentioned below, or encoding the protein as shown in the respective line in Table II, application no. 19, column 5 or 8, or homologs or fragments thereof, into a non-human organism alone or in combination with other genes, it is possible not only to increase the biosynthetic flux towards the end product, but also to increase, modify or create de novo an advantageous, preferably novel metabolites composition in the non-human organism, e.g. an advantageous composition comprising a higher content of (from a viewpoint of nutritional physiology limited) bis-glyceryl phosphate (polar fraction), glycerol (lipid fraction), glycerol (polar fraction), glycerol phosphate (lipid fraction), glycerol-3-phosphate (polar fraction), or triacylglycerol and if desired other lipids, and/or other metabolites, in free or bound form.

[100134] for the disclosure of this paragraph see [0129.1.7.7] above

[100135] Accordingly, in one embodiment, the process according to the invention relates to a process, which comprises:
[100136] (a) providing a non-human organism, preferably a microorganism, a plant cell, a plant tissue, a plant or a part thereof, more preferably a microorganism, a plant tissue, a plant or a part thereof;
[100137] (b) increasing the activity of a protein as shown in the respective line in Table II, application no. 19, column 3 or of a polypeptide being encoded by the respective nucleic acid molecule of the present invention and described below, e.g. conferring an increase of the fine chemical bis-glyceryl phosphate (polar fraction), glycerol (lipid fraction), glycerol (polar fraction), glycerol phosphate (lipid fraction), glycerol-3-phosphate (polar fraction), or triacylglycerol, respectively, in the non-human organism, preferably in the microorganism, the plant cell, the plant tissue, the plant or a part thereof, more preferably a microorganism, a plant tissue, a plant or a part thereof, especially cytoplasmic or in an organelle, like plastids or mitochondria,
[100138] (c) growing the non-human organism, preferably the microorganism, the plant cell, the plant tissue, the plant or a part thereof, under conditions which permit the production of said chemical in the non-human organism, preferably the microorganism, the plant cell, the plant tissue, the plant or a part thereof; and
[100139] (d) if desired, recovering, optionally isolating, said fine chemical, in free and/or bound form, and, optionally further free and/or bound lipids, and/or other metabolites synthesized by the non-human organism, the microorganism, the plant cell, the plant tissue, the plant or a part thereof.

[100140] [0131.1.7.19] to [0133.1.7.19] for the disclosure of these paragraphs see [0131.1.7.7] to [0133.1.7.7] above

[100141] The non-human organism, in particular the microorganism, plant or part thereof, is advantageously grown in such a way that it is not only possible to recover, if desired to isolate the free or bound fine chemical or the free and bound fine chemical but as option it is also possible to produce, recover and, if desired to isolate, other free or/and bound glycerol derivates and/or lipids and/or other metabolites. The organisms are harvested in a manner known per se and appropriate for the particular organism. Microorganisms such as bacteria, mosses, yeasts and fungi or plant cells which are cultured in liquid media by fermentation may be removed, for example, by centrifugation, decanting or filtration. Plants are grown on solid media in a manner known per se and harvested accordingly.

[100142] [0135.1.7.19] to [0139.1.7.19] for the disclosure of these paragraphs see [0135.1.7.7] to [0139.1.7.7] above

[100143] In another embodiment, the invention relates to a (isolated) nucleic acid molecule, which comprises a nucleic acid molecule selected from the group consisting of:
[100144] (a) a nucleic acid molecule encoding a polypeptide shown in Table II, application no. 19, column 5 or 8, preferably shown in Table II B, application no. 19, column 8, or a homolog or a fragment thereof;
[100145] (b) a nucleic acid molecule shown in Table I, application no. 19, column 5 or 8, preferably shown in Table I B, application no. 19, column 8, preferably the coding region thereof, or a homolog or a fragment thereof;
[100146] (c) a nucleic acid molecule, which, as a result of the degeneracy of the genetic code, can be derived from a polypeptide sequence depicted in column 5 or 8 of Table II, application no. 19, preferably in column 8 of Table II B, application no. 19;
[100147] (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 19, preferably shown in column 8 of Table I B, application no. 19, preferably the coding region thereof;
[100148] (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
[100149] (f) a nucleic acid molecule which hybridizes with a nucleic acid molecule of (a), (b), (c), (d) or (e) under stringent hybridization conditions;
[100150] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
[100151] (h) a nucleic acid molecule encoding a polypeptide comprising the consensus sequence or one or more polypeptide motifs as shown in column 8 of Table IV, application no. 19;
[100152] (i) a nucleic acid molecule encoding a polypeptide having the activity represented by a respective protein as depicted in column 5 of Table II, application no. 19;
[100153] (j) a nucleic acid molecule comprising a polynucleotide which is obtained by amplifying a cDNA library or a genomic library using the primers in Table III, application no. 19, column 8;
[100154] and
[100155] (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
[100156] or a nucleic acid molecule comprising a sequence which is complementary thereto.
   (All the references to Tables I to IV are references to the corresponding lines of Tables I to IV.)

[100157] In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 19.

[100158] In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[100159] In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II application no. 19 and confers a generation or an increase of the respective fine chemical in a non-human organism or a part thereof as compared to a corresponding wild-type non-human organism or part thereof.

[100160] In another embodiment, the invention relates to a (isolated) nucleic acid molecule, which comprises a nucleic acid molecule selected from the group consisting of:
[100161] (a) a nucleic acid molecule encoding a polypeptide shown in Table II, column 5 or 8, application no. 19, preferably shown in Table II A, application no. 19, in column 5 or in Table II A, application no. 19, column 8 or in Table II B, application no. 19, column 8, or a homolog or a fragment thereof;
[100162] (b) a nucleic acid molecule shown in Table I, application no. 19, column 5 or 8, preferably shown in Table I A, application no. 19, in column 5 or in Table I A, application no. 19, column 8 or in Table I B, application no. 19, column 8, preferably the coding region thereof, or a homolog or a fragment thereof;
[100163] (c) a nucleic acid molecule, which, as a result of the degeneracy of the genetic code, can be derived from a polypeptide sequence depicted in column 5 or 8 of Table II, application no. 19, preferably shown in Table II A, application no. 19, in column 5 or in Table II A, application no. 19, column 8 or in Table II B, application no. 19, column 8;
[100164] (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 19, preferably shown in Table I A, application no. 19, in column 5 or in Table I A, application no. 19, column 8 or in Table I B column 8, preferably the coding region thereof;
[100165] (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
[100166] (f) a nucleic acid molecule which hybridizes with a nucleic acid molecule of (a), (b), (c), (d) or (e) under stringent hybridization conditions;
[100167] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
[100168] (h) a nucleic acid molecule encoding a polypeptide comprising the consensus sequence or one or more polypeptide motifs as shown in column 8 of Table IV, application no. 19;
[100169] (i) a nucleic acid molecule encoding a polypeptide having the activity represented by a respective protein as depicted in column 5 of Table II, application no. 19;
[100170] (j) a nucleic acid molecule comprising a polynucleotide which is obtained by amplifying a cDNA library or a genomic library using the primers in Table III, application no. 19, column 8;
[100171] and
[100172] (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt,500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
[100173] or a nucleic acid molecule comprising a sequence which is complementary thereto; whereby, preferably, the nucleic acid molecule according to (a), (b), (c), (d), (e), (f), (g), (h), (i), (j) or (k) distinguishes over the sequence depicted in Table I A and/or I B, application no. 19, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 19, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 19, column 5 or 8, or the coding region thereof, respectively. In a further embodiment the nucleic acid molecule does not encode the polypeptide sequence shown in Table II A and/or II B, application no. 19, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 19, column 5 or 8 and does not encode a protein of the sequence shown in Table II A and/or II B, application no. 19, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 19, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 19, column 5 or 8 but less than 100%, preferably less than 99.999%, 99.99%, 99.9%, 99%, 98%, 97%, 96% or 95% identical to the sequence shown in Table II A and/or II B, application no. 19, column 5 or 8.
   (All the references to Tables I to IV are references to the corresponding lines of Tables I to IV.)

[100174] In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 19.

[100175] In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[100176] In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II, application no. 19, and confers a generation or an increase of the respective fine chemical in a non-human organism or a part thereof as compared to a corresponding wild-type non-human organism or part thereof.

[100177] [0144.1.7.19] to [0155.1.7.19] for the disclosure of theses paragraphs see [0144.1.7.7] to [0155.1.7.7] above

[100178] In a preferred embodiment a nucleic acid construct, for example an expression cassette, comprises upstream, i.e. at the 5′ end of the encoding sequence, a promoter and downstream, i.e. at the 3′ end, a polyadenylation signal and optionally other regulatory elements which are operably linked to the intervening encoding sequence with one of the nucleic acids as depicted in the respective line in Table I, application no. 19, column 5 or 8, preferably the coding region thereof, or a homolog or a fragment thereof. By an operable linkage is meant the sequential arrangement of promoter, encoding sequence, terminator, optionally other regulatory elements and optionally targeting sequences in such a way that each of the regulatory elements/targeting sequence can fulfill its function in the expression of the encoding sequence in due manner. In one embodiment the sequences preferred for operable linkage are targeting sequences for ensuring subcellular localization in organelles, like plastids or mitochondria. However, targeting sequences for ensuring subcellular localization in the endoplasmic reticulum (=ER), in the nucleus, in oil corpuscles or other compartments may also be employed as well as translation promoters such as the 5′ lead sequence in tobacco mosaic virus (Gallie et al., Nucl. Acids Res. 15, 8693 (1987)).

[100179] for the disclosure of this paragraph see [0157.1.7.7] above

[100180] In an embodiment, the invention relates to an expression cassette, comprising
[100181] 1) a promoter, preferably a promoter selected from the group consisting of Big35S, PCUbi, Super and USP;
[100182] 2) a nucleic acid molecule selected from the group consisting of
   [100183] (a) a nucleic acid molecule encoding a polypeptide shown in Table II, application no. 19, column 5 or 8, preferably shown in Table II A, application no. 19, column 5, or in Table II A, application no. 19, column 8, or in Table II B, application no. 19, column 8, or a homolog or a fragment thereof;
   [100184] (b) a nucleic acid molecule shown in Table I, application no. 19, column 5 or 8, preferably shown in Table I A, application no. 19, column 5, or in Table I A, application no. 19, column 8, or in Table I B, application no. 19, column 8, preferably the coding region thereof, or a homolog or a fragment thereof;
   [100185] (c) a nucleic acid molecule, which, as a result of the degeneracy of the genetic code, can be derived from a polypeptide sequence depicted in column 5 or 8 of Table II, application no. 19, preferably shown in Table II A, application no. 19, column 5, or in Table II A, application no. 19, column 8, or in Table II B, application no. 19, column 8;
   [100186] (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 19, preferably shown in Table I A, application no. 19, column 5, or in Table I A, application no. 19, column 8, or in Table I B, application no. 19, column 8, preferably the coding region thereof;
   [100187] (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   [100188] (f) a nucleic acid molecule which hybridizes with a nucleic acid molecule of (a), (b), (c), (d) or (e) under stringent hybridization conditions;
   [100189] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   [100190] (h) a nucleic acid molecule encoding a polypeptide comprising the consensus sequence or one or more polypeptide motifs as shown in column 8 of Table IV, application no. 19;
   [100191] (i) a nucleic acid molecule encoding a polypeptide having the activity represented by a respective protein as depicted in column 5 of Table II, application no. 19;
   [100192] (j) a nucleic acid molecule comprising a polynucleotide which is obtained by amplifying a cDNA library or a genomic library using the primers in Table III, application no. 19, column 8;
   [100193] and
   [100194] nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   [100195] or a nucleic acid molecule comprising a sequence which is complementary thereto. and, optionally
[100196] 3) nucleic acid molecule encoding a transit peptide, preferably encoding a plastidal transit peptide or a mitochondrial transit peptide;
   which are operable linked.
   (All the references to Tables I to IV are references to the corresponding lines of Tables I to IV.)

[100197] In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 19.

[100198] In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[100199] In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 19 and confers a generation or an increase of the respective fine chemical in a non-human organism or a part thereof as compared to a corresponding wild-type non-human organism or part thereof.

[100200] In another embodiment thereof the expression cassette comprises 3) a nucleic acid molecule encoding a transit peptide, preferably encoding a plastidal transit peptide, especially in case in column 6 of Table I, application no. 19, in the same line as of the respective nucleic acid molecule according to 2) “plastidic” is depicted, or encoding a mitochondrial transit peptide, especially in case in column 6 of Table I, application no. 19, in the same line as of the respective nucleic acid molecule according to 2) “mitochondric” is depicted.

[100201] In another embodiment thereof the expression cassette does not comprise 3) a nucleic acid molecule encoding a transit peptide, especially in case in column 6 of Table I, application no. 19, in the same line as of the respective nucleic acid molecule according to 2) “non-targeted” is depicted.

[100202] In a further embodiment, the invention relates to an expression cassette, comprising
[100203] 1) a promoter, preferably a promoter selected from the group consisting of Big35S, PCUbi, Super and USP;
[100204] 2) a nucleic acid molecule selected from the group consisting of
   [100205] (a) a nucleic acid molecule encoding a polypeptide shown in Table II, application no. 19, column 5 or 8, preferably shown in Table II A, application no. 19, column 5, or in Table II A, application no. 19, column 8, or in Table II B, application no. 19, column 8, or a homolog or a fragment thereof;
   [100206] (b) a nucleic acid molecule shown in Table I, application no. 19, column 5 or 8, preferably shown in Table I A, application no. 19, column 5, or in Table I A, application no. 19, column 8, or in Table I B, application no. 19, column 8, preferably the coding region thereof, or a homolog or a fragment thereof;
   [100207] (c) a nucleic acid molecule, which, as a result of the degeneracy of the genetic code, can be derived from a polypeptide sequence depicted in column 5 or 8 of Table II, application no. 19, preferably shown in Table II A, application no. 19, column 5, or in Table II A, application no. 19, column 8, or in Table II B, application no. 19, column 8;
   [100208] (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 19, preferably shown in Table I A, application no. 19, column 5, or in Table I A, application no. 19, column 8,or in Table I B, application no. 19, column 8, preferably the coding region thereof;
   [100209] (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   [100210] (f) a nucleic acid molecule which hybridizes with a nucleic acid molecule of (a), (b), (c), (d) or (e) under stringent hybridization conditions;
   [100211] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   [100212] (h) a nucleic acid molecule encoding a polypeptide comprising the consensus sequence or one or more polypeptide motifs as shown in column 8 of Table IV, application no. 19;
   [100213] (i) a nucleic acid molecule encoding a polypeptide having the activity represented by a respective protein as depicted in column 5 of Table II, application no. 19;
   [100214] (j) a nucleic acid molecule comprising a polynucleotide which is obtained by amplifying a cDNA library or a genomic library using the primers in Table III, application no. 19, column 8;
   [100215] and
   [100216] (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   [100217] or a nucleic acid molecule comprising a sequence which is complementary thereto; and, optionally
[100218] 3) nucleic acid molecule encoding a transit peptide, preferably encoding a plastidal transit peptide or a mitochondrial transit peptide;
   which are operable linked and
   whereby, preferably, the nucleic acid molecule according to 2 (a), (b), (c), (d), (e), (f), (g), (h), (i), (j) or (k) distinguishes over the sequence depicted in Table I A and/or I B, application no. 19, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention according to 2) does not consist of the sequence shown in Table I A and/or I B, application no. 19, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 19, column 5 or 8, or the coding region thereof, respectively. In a further embodiment the nucleic acid molecule does not encode the polypeptide sequence shown in Table II A and/or II B, application no. 19, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 19, column 5 or 8 and does not encode a protein of the sequence shown in Table II A and/or II B, application no. 19, column 5 or 8. Accordingly, in one embodiment, the protein encoded by a sequence of a nucleic acid according to 2(a), (b), (c), (d), (e), (f), (g), (h), (i), (j) or (k) does not consist of the sequence shown in Table I A and/or I B, application no. 19, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 19, column 5 or 8 but less than 100%, 99.999%, 99.99%, 99.9%, 99%, 98%, 97%, 96% or 95% identical to the sequence shown in Table II A and/or II B, application no. 19, column 5 or 8.
   (All the references to Tables I to IV are references to the corresponding lines of Tables I to IV.)

[100219] In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 19.

[100220] In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[100221] In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 19 and confers a generation or an increase of the respective fine chemical in a non-human organism or a part thereof as compared to a corresponding wild-type non-human organism or part thereof.

[100222] In another embodiment thereof the expression cassette comprises 3) a nucleic acid molecule encoding a transit peptide, preferably encoding a plastidal transit peptide, especially in case in column 6 of Table I, application no. 19, in the same line as of the respective nucleic acid molecule according to 2) “plastidic” is depicted, or encoding a mitochondrial transit peptide, especially in case in column 6 of Table I, application no. 19, in the same line as of the respective nucleic acid molecule according to 2) “mitochondric” is depicted.

[100223] In another embodiment thereof the expression cassette does not comprise 3) a nucleic acid molecule encoding a transit peptide, especially in case in column 6 of Table I, application no. 19, in the same line as of the respective nucleic acid molecule according to 2) “non-targeted” is depicted.

[100224] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17451, or a homolog or fragment thereof, or a homolog thereof being depicted in Table I, application no. 19, column 8, in the same line as SEQ ID NO. 17451, preferably the coding region thereof, a homolog or a fragment thereof; which are operable linked.

[100225] In an embodiment thereof the expression cassette comprises in addition c) a nucleic acid molecule encoding a transit peptide, especially a plastidal transit petide in case in Table I, application no. 19, in column 6 in the same line as SEQ ID NO. 17451 is depicted, plastidic is mentioned, or a mitochondrial transit peptide in case in Table I, application no. 19, in column 6 in the same line as SEQ ID NO. 17451 is depicted, mitochondric is mentioned; which is operable linked, too.

[100226] In another embodiment thereof the expression cassette does not comprise an additional transit peptide in case in Table I, application no. 19, in column 6 in the same line as SEQ ID NO. 17451 is depicted, non-targeted is mentioned.

[100227] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 102086, or a homolog or fragment thereof, or a homolog thereof being depicted in Table I, application no. 19, column 8, in the same line as SEQ ID NO. 102086, preferably the coding region thereof, a homolog or a fragment thereof; which are operable linked.

[100228] In an embodiment thereof the expression cassette comprises in addition c) a nucleic acid molecule encoding a transit peptide, especially a plastidal transit petide in case in Table I, application no. 19, in column 6 in the same line as SEQ ID NO. 102086 is depicted, plastidic is mentioned, or a mitochondrial transit peptide in case in Table I, application no. 19, in column 6 in the same line as SEQ ID NO. 102086 is depicted, mitochondric is mentioned; which is operable linked, too.

[100229] In another embodiment thereof the expression cassette does not comprise an additional transit peptide in case in Table I, application no. 19, in column 6 in the same line as SEQ ID NO. 102086 is depicted, non-targeted is mentioned.

[100230] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 17968, or a homolog or fragment thereof, or a homolog thereof being depicted in Table I, application no. 19, column 8, in the same line as SEQ ID NO. 17968, preferably the coding region thereof, a homolog or a fragment thereof; which are operable linked.

[100231] In an embodiment thereof the expression cassette comprises in addition c) a nucleic acid molecule encoding a transit peptide, especially a plastidal transit petide in case in Table I, application no. 19, in column 6 in the same line as SEQ ID NO. 17968 is depicted, plastidic is mentioned, or a mitochondrial transit peptide in case in Table I, application no. 19, in column 6 in the same line as SEQ ID NO. 17968 is depicted, mitochondric is mentioned; which is operable linked, too.

[100232] In another embodiment thereof the expression cassette does not comprise an additional transit peptide in case in Table I, application no. 19, in column 6 in the same line as SEQ ID NO. 17968 is depicted, non-targeted is mentioned.

[100233] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 19364, or a homolog or fragment thereof, or a homolog thereof being depicted in Table I, application no. 19, column 8, in the same line as SEQ ID NO. 19364, preferably the coding region thereof, a homolog or a fragment thereof; which are operable linked.

[100234] In an embodiment thereof the expression cassette comprises in addition c) a nucleic acid molecule encoding a transit peptide, especially a plastidal transit petide in case in Table I, application no. 19, in column 6 in the same line as SEQ ID NO. 19364 is depicted, plastidic is mentioned, or a mitochondrial transit peptide in case in Table I, application no. 19, in column 6 in the same line as SEQ ID NO. 19364 is depicted, mitochondric is mentioned; which is operable linked, too.

[100235] In another embodiment thereof the expression cassette does not comprise an additional transit peptide in case in Table I, application no. 19, in column 6 in the same line as SEQ ID NO. 19364 is depicted, non-targeted is mentioned.

[100236] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 1061, or a homolog or fragment thereof, or a homolog thereof being depicted in Table I, application no. 19, column 8, in the same line as SEQ ID NO. 1061, preferably the coding region thereof, a homolog or a fragment thereof; which are operable linked.

[100237] In an embodiment thereof the expression cassette comprises in addition c) a nucleic acid molecule encoding a transit peptide, especially a plastidal transit petide in case in Table I, application no. 19, in column 6 in the same line as SEQ ID NO. 1061 is depicted, plastidic is mentioned, or a mitochondrial transit peptide in case in Table I, application no. 19, in column 6 in the same line as SEQ ID NO. 1061 is depicted, mitochondric is mentioned; which is operable linked, too.

[100238] In another embodiment thereof the expression cassette does not comprise an additional transit peptide in case in Table I, application no. 19, in column 6 in the same line as SEQ ID NO. 1061 is depicted, non-targeted is mentioned.

[100239] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 19419, or a homolog or fragment thereof, or a homolog thereof being depicted in Table I, application no. 19, column 8, in the same line as SEQ ID NO. 19419, preferably the coding region thereof, a homolog or a fragment thereof; which are operable linked.

[100240] In an embodiment thereof the expression cassette comprises in addition c) a nucleic acid molecule encoding a transit peptide, especially a plastidal transit petide in case in Table I, application no. 19, in column 6 in the same line as SEQ ID NO. 19419 is depicted, plastidic is mentioned, or a mitochondrial transit peptide in case in Table I, application no. 19, in column 6 in the same line as SEQ ID NO. 19419 is depicted, mitochondric is mentioned; which is operable linked, too.

[100241] In another embodiment thereof the expression cassette does not comprise an additional transit peptide in case in Table I, application no. 19, in column 6 in the same line as SEQ ID NO. 19419 is depicted, non-targeted is mentioned.

[100242] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 19919, or a homolog or fragment thereof, or a homolog thereof being depicted in Table I, application no. 19, column 8, in the same line as SEQ ID NO. 19919, preferably the coding region thereof, a homolog or a fragment thereof; which are operable linked.

[100243] In an embodiment thereof the expression cassette comprises in addition c) a nucleic acid molecule encoding a transit peptide, especially a plastidal transit petide in case in Table I, application no. 19, in column 6 in the same line as SEQ ID NO. 19919 is depicted, plastidic is mentioned, or a mitochondrial transit peptide in case in Table I, application no. 19, in column 6 in the same line as SEQ ID NO. 19919 is depicted, mitochondric is mentioned; which is operable linked, too.

[100244] In another embodiment thereof the expression cassette does not comprise an additional transit peptide in case in Table I, application no. 19, in column 6 in the same line as SEQ ID NO. 19919 is depicted, non-targeted is mentioned.

[100245] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 21106, or a homolog or fragment thereof, or a homolog thereof being depicted in Table I, application no. 19, column 8, in the same line as SEQ ID NO. 21106, preferably the coding region thereof, a homolog or a fragment thereof; which are operable linked.

[100246] In an embodiment thereof the expression cassette comprises in addition c) a nucleic acid molecule encoding a transit peptide, especially a plastidal transit petide in case in Table I, application no. 19, in column 6 in the same line as SEQ ID NO. 21106 is depicted, plastidic is mentioned, or a mitochondrial transit peptide in case in Table I, application no. 19, in column 6 in the same line as SEQ ID NO. 21106 is depicted, mitochondric is mentioned; which is operable linked, too.

[100247] In another embodiment thereof the expression cassette does not comprise an additional transit peptide in case in Table I, application no. 19, in column 6 in the same line as SEQ ID NO. 21106 is depicted, non-targeted is mentioned.

[100248] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68657, or a homolog or fragment thereof, or a homolog thereof being depicted in Table I, application no. 19, column 8, in the same line as SEQ ID NO. 68657, preferably the coding region thereof, a homolog or a fragment thereof; which are operable linked.

[100249] In an embodiment thereof the expression cassette comprises in addition c) a nucleic acid molecule encoding a transit peptide, especially a plastidal transit petide in case in Table I, application no. 19, in column 6 in the same line as SEQ ID NO. 68657 is depicted, plastidic is mentioned, or a mitochondrial transit peptide in case in Table I, application no. 19, in column 6 in the same line as SEQ ID NO. 68657 is depicted, mitochondric is mentioned; which is operable linked, too.

[100250] In another embodiment thereof the expression cassette does not comprise an additional transit peptide in case in Table I, application no. 19, in column 6 in the same line as SEQ ID NO. 68657 is depicted, non-targeted is mentioned.

[100251] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 109717, or a homolog or fragment thereof, or a homolog thereof being depicted in Table I, application no. 19, column 8, in the same line as SEQ ID NO. 109717, preferably the coding region thereof, a homolog or a fragment thereof; which are operable linked.

[100252] In an embodiment thereof the expression cassette comprises in addition c) a nucleic acid molecule encoding a transit peptide, especially a plastidal transit petide in case in Table I, application no. 19, in column 6 in the same line as SEQ ID NO. 109717 is depicted, plastidic is mentioned, or a mitochondrial transit peptide in case in Table I, application no. 19, in column 6 in the same line as SEQ ID NO. 109717 is depicted, mitochondric is mentioned; which is operable linked, too.

[100253] In another embodiment thereof the expression cassette does not comprise an additional transit peptide in case in Table I, application no. 19, in column 6 in the same line as SEQ ID NO. 109717 is depicted, non-targeted is mentioned.

[100254] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 23482, or a homolog or fragment thereof, or a homolog thereof being depicted in Table I, application no. 19, column 8, in the same line as SEQ ID NO. 23482, preferably the coding region thereof, a homolog or a fragment thereof; which are operable linked.

[100255] In an embodiment thereof the expression cassette comprises in addition c) a nucleic acid molecule encoding a transit peptide, especially a plastidal transit petide in case in Table I, application no. 19, in column 6 in the same line as SEQ ID NO. 23482 is depicted, plastidic is mentioned, or a mitochondrial transit peptide in case in Table I, application no. 19, in column 6 in the same line as SEQ ID NO. 23482 is depicted, mitochondric is mentioned; which is operable linked, too.

[100256] In another embodiment thereof the expression cassette does not comprise an additional transit peptide in case in Table I, application no. 19, in column 6 in the same line as SEQ ID NO. 23482 is depicted, non-targeted is mentioned.

[100257] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 3279, or a homolog or fragment thereof, or a homolog thereof being depicted in Table I, application no. 19, column 8, in the same line as SEQ ID NO. 3279, preferably the coding region thereof, a homolog or a fragment thereof; which are operable linked.

[100258] In an embodiment thereof the expression cassette comprises in addition c) a nucleic acid molecule encoding a transit peptide, especially a plastidal transit petide in case in Table I, application no. 19, in column 6 in the same line as SEQ ID NO. 3279 is depicted, plastidic is mentioned, or a mitochondrial transit peptide in case in Table I, application no. 19, in column 6 in the same line as SEQ ID NO. 3279 is depicted, mitochondric is mentioned; which is operable linked, too.

[100259] In another embodiment thereof the expression cassette does not comprise an additional transit peptide in case in Table I, application no. 19, in column 6 in the same line as SEQ ID NO. 3279 is depicted, non-targeted is mentioned.

[100260] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68849, or a homolog or fragment thereof, or a homolog thereof being depicted in Table I, application no. 19, column 8, in the same line as SEQ ID NO. 68849, preferably the coding region thereof, a homolog or a fragment thereof; which are operable linked.

[100261] In an embodiment thereof the expression cassette comprises in addition c) a nucleic acid molecule encoding a transit peptide, especially a plastidal transit petide in case in Table I, application no. 19, in column 6 in the same line as SEQ ID NO. 68849 is depicted, plastidic is mentioned, or a mitochondrial transit peptide in case in Table I, application no. 19, in column 6 in the same line as SEQ ID NO. 68849 is depicted, mitochondric is mentioned; which is operable linked, too.

[100262] In another embodiment thereof the expression cassette does not comprise an additional transit peptide in case in Table I, application no. 19, in column 6 in the same line as SEQ ID NO. 68849 is depicted, non-targeted is mentioned.

[100263] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 24311, or a homolog or fragment thereof, or a homolog thereof being depicted in Table I, application no. 19, column 8, in the same line as SEQ ID NO. 24311, preferably the coding region thereof, a homolog or a fragment thereof; which are operable linked.

[100264] In an embodiment thereof the expression cassette comprises in addition c) a nucleic acid molecule encoding a transit peptide, especially a plastidal transit petide in case in Table I, application no. 19, in column 6 in the same line as SEQ ID NO. 24311 is depicted, plastidic is mentioned, or a mitochondrial transit peptide in case in Table I, application no. 19, in column 6 in the same line as SEQ ID NO. 24311 is depicted, mitochondric is mentioned; which is operable linked, too.

[100265] In another embodiment thereof the expression cassette does not comprise an additional transit peptide in case in Table I, application no. 19, in column 6 in the same line as SEQ ID NO. 24311 is depicted, non-targeted is mentioned.

[100266] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4348, or a homolog or fragment thereof, or a homolog thereof being depicted in Table I, application no. 19, column 8, in the same line as SEQ ID NO. 4348, preferably the coding region thereof, a homolog or a fragment thereof; which are operable linked.

[100267] In an embodiment thereof the expression cassette comprises in addition c) a nucleic acid molecule encoding a transit peptide, especially a plastidal transit petide in case in Table I, application no. 19, in column 6 in the same line as SEQ ID NO. 4348 is depicted, plastidic is mentioned, or a mitochondrial transit peptide in case in Table I, application no. 19, in column 6 in the same line as SEQ ID NO. 4348 is depicted, mitochondric is mentioned; which is operable linked, too.

[100268] In another embodiment thereof the expression cassette does not comprise an additional transit peptide in case in Table I, application no. 19, in column 6 in the same line as SEQ ID NO. 4348 is depicted, non-targeted is mentioned.

[100269] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 24438, or a homolog or fragment thereof, or a homolog thereof being depicted in Table I, application no. 19, column 8, in the same line as SEQ ID NO. 24438, preferably the coding region thereof, a homolog or a fragment thereof; which are operable linked.

[100270] In an embodiment thereof the expression cassette comprises in addition c) a nucleic acid molecule encoding a transit peptide, especially a plastidal transit petide in case in Table I, application no. 19, in column 6 in the same line as SEQ ID NO. 24438 is depicted, plastidic is mentioned, or a mitochondrial transit peptide in case in Table I, application no. 19, in column 6 in the same line as SEQ ID NO. 24438 is depicted, mitochondric is mentioned; which is operable linked, too.

[100271] In another embodiment thereof the expression cassette does not comprise an additional transit peptide in case in Table I, application no. 19, in column 6 in the same line as SEQ ID NO. 24438 is depicted, non-targeted is mentioned.

[100272] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 4904, or a homolog or fragment thereof, or a homolog thereof being depicted in Table I, application no. 19, column 8, in the same line as SEQ ID NO. 4904, preferably the coding region thereof, a homolog or a fragment thereof; which are operable linked.

[100273] In an embodiment thereof the expression cassette comprises in addition c) a nucleic acid molecule encoding a transit peptide, especially a plastidal transit petide in case in Table I, application no. 19, in column 6 in the same line as SEQ ID NO. 4904 is depicted, plastidic is mentioned, or a mitochondrial transit peptide in case in Table I, application no. 19, in column 6 in the same line as SEQ ID NO. 4904 is depicted, mitochondric is mentioned; which is operable linked, too.

[100274] In another embodiment thereof the expression cassette does not comprise an additional transit peptide in case in Table I, application no. 19, in column 6 in the same line as SEQ ID NO. 4904 is depicted, non-targeted is mentioned.

[100275] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 5318, or a homolog or fragment thereof, or a homolog thereof being depicted in Table I, application no. 19, column 8, in the same line as SEQ ID NO. 5318, preferably the coding region thereof, a homolog or a fragment thereof; which are operable linked.

[100276] In an embodiment thereof the expression cassette comprises in addition c) a nucleic acid molecule encoding a transit peptide, especially a plastidal transit petide in case in Table I, application no. 19, in column 6 in the same line as SEQ ID NO. 5318 is depicted, plastidic is mentioned, or a mitochondrial transit peptide in case in Table I, application no. 19, in column 6 in the same line as SEQ ID NO. 5318 is depicted, mitochondric is mentioned; which is operable linked, too.

[100277] In another embodiment thereof the expression cassette does not comprise an additional transit peptide in case in Table I, application no. 19, in column 6 in the same line as SEQ ID NO. 5318 is depicted, non-targeted is mentioned.

[100278] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 25498, or a homolog or fragment thereof, or a homolog thereof being depicted in Table I, application no. 19, column 8, in the same line as SEQ ID NO. 25498, preferably the coding region thereof, a homolog or a fragment thereof; which are operable linked.

[100279] In an embodiment thereof the expression cassette comprises in addition c) a nucleic acid molecule encoding a transit peptide, especially a plastidal transit petide in case in Table I, application no. 19, in column 6 in the same line as SEQ ID NO. 25498 is depicted, plastidic is mentioned, or a mitochondrial transit peptide in case in Table I, application no. 19, in column 6 in the same line as SEQ ID NO. 25498 is depicted, mitochondric is mentioned; which is operable linked, too.

[100280] In another embodiment thereof the expression cassette does not comprise an additional transit peptide in case in Table I, application no. 19, in column 6 in the same line as SEQ ID NO. 25498 is depicted, non-targeted is mentioned.

[100281] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 70038, or a homolog or fragment thereof, or a homolog thereof being depicted in Table I, application no. 19, column 8, in the same line as SEQ ID NO. 70038, preferably the coding region thereof, a homolog or a fragment thereof; which are operable linked.

[100282] In an embodiment thereof the expression cassette comprises in addition c) a nucleic acid molecule encoding a transit peptide, especially a plastidal transit petide in case in Table I, application no. 19, in column 6 in the same line as SEQ ID NO. 70038 is depicted, plastidic is mentioned, or a mitochondrial transit peptide in case in Table I, application no. 19, in column 6 in the same line as SEQ ID NO. 70038 is depicted, mitochondric is mentioned; which is operable linked, too.

[100283] In another embodiment thereof the expression cassette does not comprise an additional transit peptide in case in Table I, application no. 19, in column 6 in the same line as SEQ ID NO. 70038 is depicted, non-targeted is mentioned.

[100284] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 70715, or a homolog or fragment thereof, or a homolog thereof being depicted in Table I, application no. 19, column 8, in the same line as SEQ ID NO. 70715, preferably the coding region thereof, a homolog or a fragment thereof; which are operable linked.

[100285] In an embodiment thereof the expression cassette comprises in addition c) a nucleic acid molecule encoding a transit peptide, especially a plastidal transit petide in case in Table I, application no. 19, in column 6 in the same line as SEQ ID NO. 70715 is depicted, plastidic is mentioned, or a mitochondrial transit peptide in case in Table I, application no. 19, in column 6 in the same line as SEQ ID NO. 70715 is depicted, mitochondric is mentioned; which is operable linked, too.

[100286] In another embodiment thereof the expression cassette does not comprise an additional transit peptide in case in Table I, application no. 19, in column 6 in the same line as SEQ ID NO. 70715 is depicted, non-targeted is mentioned.

[100287] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 26196, or a homolog or fragment thereof, or a homolog thereof being depicted in Table I, application no. 19, column 8, in the same line as SEQ ID NO. 26196, preferably the coding region thereof, a homolog or a fragment thereof; which are operable linked.

[100288] In an embodiment thereof the expression cassette comprises in addition c) a nucleic acid molecule encoding a transit peptide, especially a plastidal transit petide in case in Table I, application no. 19, in column 6 in the same line as SEQ ID NO. 26196 is depicted, plastidic is mentioned, or a mitochondrial transit peptide in case in Table I, application no. 19, in column 6 in the same line as SEQ ID NO. 26196 is depicted, mitochondric is mentioned; which is operable linked, too.

[100289] In another embodiment thereof the expression cassette does not comprise an additional transit peptide in case in Table I, application no. 19, in column 6 in the same line as SEQ ID NO. 26196 is depicted, non-targeted is mentioned.

[100290] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 114083, or a homolog or fragment thereof, or a homolog thereof being depicted in Table I, application no. 19, column 8, in the same line as SEQ ID NO. 114083, preferably the coding region thereof, a homolog or a fragment thereof; which are operable linked.

[100291] In an embodiment thereof the expression cassette comprises in addition c) a nucleic acid molecule encoding a transit peptide, especially a plastidal transit petide in case in Table I, application no. 19, in column 6 in the same line as SEQ ID NO. 114083 is depicted, plastidic is mentioned, or a mitochondrial transit peptide in case in Table I, application no. 19, in column 6 in the same line as SEQ ID NO. 114083 is depicted, mitochondric is mentioned; which is operable linked, too.

[100292] In another embodiment thereof the expression cassette does not comprise an additional transit peptide in case in Table I, application no. 19, in column 6 in the same line as SEQ ID NO. 114083 is depicted, non-targeted is mentioned.

[100293] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 114198, or a homolog or fragment thereof, or a homolog thereof being depicted in Table I, application no. 19, column 8, in the same line as SEQ ID NO. 114198, preferably the coding region thereof, a homolog or a fragment thereof; which are operable linked.

[100294] In an embodiment thereof the expression cassette comprises in addition c) a nucleic acid molecule encoding a transit peptide, especially a plastidal transit petide in case in Table I, application no. 19, in column 6 in the same line as SEQ ID NO. 114198 is depicted, plastidic is mentioned, or a mitochondrial transit peptide in case in Table I, application no. 19, in column 6 in the same line as SEQ ID NO. 114198 is depicted, mitochondric is mentioned; which is operable linked, too.

[100295] In another embodiment thereof the expression cassette does not comprise an additional transit peptide in case in Table I, application no. 19, in column 6 in the same line as SEQ ID NO. 114198 is depicted, non-targeted is mentioned.

[100296] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6040, or a homolog or fragment thereof, or a homolog thereof being depicted in Table I, application no. 19, column 8, in the same line as SEQ ID NO. 6040, preferably the coding region thereof, a homolog or a fragment thereof; which are operable linked.

[100297] In an embodiment thereof the expression cassette comprises in addition c) a nucleic acid molecule encoding a transit peptide, especially a plastidal transit petide in case in Table I, application no. 19, in column 6 in the same line as SEQ ID NO. 6040 is depicted, plastidic is mentioned, or a mitochondrial transit peptide in case in Table I, application no. 19, in column 6 in the same line as SEQ ID NO. 6040 is depicted, mitochondric is mentioned; which is operable linked, too.

[100298] In another embodiment thereof the expression cassette does not comprise an additional transit peptide in case in Table I, application no. 19, in column 6 in the same line as SEQ ID NO. 6040 is depicted, non-targeted is mentioned.

[100299] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 29500, or a homolog or fragment thereof, or a homolog thereof being depicted in Table I, application no. 19, column 8, in the same line as SEQ ID NO. 29500, preferably the coding region thereof, a homolog or a fragment thereof; which are operable linked.

[100300] In an embodiment thereof the expression cassette comprises in addition c) a nucleic acid molecule encoding a transit peptide, especially a plastidal transit petide in case in Table I, application no. 19, in column 6 in the same line as SEQ ID NO. 29500 is depicted, plastidic is mentioned, or a mitochondrial transit peptide in case in Table I, application no. 19, in column 6 in the same line as SEQ ID NO. 29500 is depicted, mitochondric is mentioned; which is operable linked, too.

[100301] In another embodiment thereof the expression cassette does not comprise an additional transit peptide in case in Table I, application no. 19, in column 6 in the same line as SEQ ID NO. 29500 is depicted, non-targeted is mentioned.

[100302] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 111155, or a homolog or fragment thereof, or a homolog thereof being depicted in Table I, application no. 19, column 8, in the same line as SEQ ID NO. 111155, preferably the coding region thereof, a homolog or a fragment thereof; which are operable linked.

[100303] In an embodiment thereof the expression cassette comprises in addition c) a nucleic acid molecule encoding a transit peptide, especially a plastidal transit petide in case in Table I, application no. 19, in column 6 in the same line as SEQ ID NO. 111155 is depicted, plastidic is mentioned, or a mitochondrial transit peptide in case in Table I, application no. 19, in column 6 in the same line as SEQ ID NO. 111155 is depicted, mitochondric is mentioned; which is operable linked, too.

[100304] In another embodiment thereof the expression cassette does not comprise an additional transit peptide in case in Table I, application no. 19, in column 6 in the same line as SEQ ID NO. 111155 is depicted, non-targeted is mentioned.

[100305] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 114231, or a homolog or fragment thereof, or a homolog thereof being depicted in Table I, application no. 19, column 8, in the same line as SEQ ID NO. 114231, preferably the coding region thereof, a homolog or a fragment thereof; which are operable linked.

[100306] In an embodiment thereof the expression cassette comprises in addition c) a nucleic acid molecule encoding a transit peptide, especially a plastidal transit petide in case in Table I, application no. 19, in column 6 in the same line as SEQ ID NO. 114231 is depicted, plastidic is mentioned, or a mitochondrial transit peptide in case in Table I, application no. 19, in column 6 in the same line as SEQ ID NO. 114231 is depicted, mitochondric is mentioned; which is operable linked, too.

[100307] In another embodiment thereof the expression cassette does not comprise an additional transit peptide in case in Table I, application no. 19, in column 6 in the same line as SEQ ID NO. 114231 is depicted, non-targeted is mentioned.

[100308] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 73719, or a homolog or fragment thereof, or a homolog thereof being depicted in Table I, application no. 19, column 8, in the same line as SEQ ID NO. 73719, preferably the coding region thereof, a homolog or a fragment thereof; which are operable linked.

[100309] In an embodiment thereof the expression cassette comprises in addition c) a nucleic acid molecule encoding a transit peptide, especially a plastidal transit petide in case in Table I, application no. 19, in column 6 in the same line as SEQ ID NO. 73719 is depicted, plastidic is mentioned, or a mitochondrial transit peptide in case in Table I, application no. 19, in column 6 in the same line as SEQ ID NO. 73719 is depicted, mitochondric is mentioned; which is operable linked, too.

[100310] In another embodiment thereof the expression cassette does not comprise an additional transit peptide in case in Table I, application no. 19, in column 6 in the same line as SEQ ID NO. 73719 is depicted, non-targeted is mentioned.

[100311] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 6510, or a homolog or fragment thereof, or a homolog thereof being depicted in Table I, application no. 19, column 8, in the same line as SEQ ID NO. 6510, preferably the coding region thereof, a homolog or a fragment thereof; which are operable linked.

[100312] In an embodiment thereof the expression cassette comprises in addition c) a nucleic acid molecule encoding a transit peptide, especially a plastidal transit petide in case in Table I, application no. 19, in column 6 in the same line as SEQ ID NO. 6510 is depicted, plastidic is mentioned, or a mitochondrial transit peptide in case in Table I, application no. 19, in column 6 in the same line as SEQ ID NO. 6510 is depicted, mitochondric is mentioned; which is operable linked, too.

[100313] In another embodiment thereof the expression cassette does not comprise an additional transit peptide in case in Table I, application no. 19, in column 6 in the same line as SEQ ID NO. 6510 is depicted, non-targeted is mentioned.

[100314] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 32648, or a homolog or fragment thereof, or a homolog thereof being depicted in Table I, application no. 19, column 8, in the same line as SEQ ID NO. 32648, preferably the coding region thereof, a homolog or a fragment thereof; which are operable linked.

[100315] In an embodiment thereof the expression cassette comprises in addition c) a nucleic acid molecule encoding a transit peptide, especially a plastidal transit petide in case in Table I, application no. 19, in column 6 in the same line as SEQ ID NO. 32648 is depicted, plastidic is mentioned, or a mitochondrial transit peptide in case in Table I, application no. 19, in column 6 in the same line as SEQ ID NO. 32648 is depicted, mitochondric is mentioned; which is operable linked, too.

[100316] In another embodiment thereof the expression cassette does not comprise an additional transit peptide in case in Table I, application no. 19, in column 6 in the same line as SEQ ID NO. 32648 is depicted, non-targeted is mentioned.

[100317] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 103433, or a homolog or fragment thereof, or a homolog thereof being depicted in Table I, application no. 19, column 8, in the same line as SEQ ID NO. 103433, preferably the coding region thereof, a homolog or a fragment thereof; which are operable linked.

[100318] In an embodiment thereof the expression cassette comprises in addition c) a nucleic acid molecule encoding a transit peptide, especially a plastidal transit petide in case in Table I, application no. 19, in column 6 in the same line as SEQ ID NO. 103433 is depicted, plastidic is mentioned, or a mitochondrial transit peptide in case in Table I, application no. 19, in column 6 in the same line as SEQ ID NO. 103433 is depicted, mitochondric is mentioned; which is operable linked, too.

[100319] In another embodiment thereof the expression cassette does not comprise an additional transit peptide in case in Table I, application no. 19, in column 6 in the same line as SEQ ID NO. 103433 is depicted, non-targeted is mentioned.

[100320] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 34889, or a homolog or fragment thereof, or a homolog thereof being depicted in Table I, application no. 19, column 8, in the same line as SEQ ID NO. 34889, preferably the coding region thereof, a homolog or a fragment thereof; which are operable linked.

[100321] In an embodiment thereof the expression cassette comprises in addition c) a nucleic acid molecule encoding a transit peptide, especially a plastidal transit petide in case in Table I, application no. 19, in column 6 in the same line as SEQ ID NO. 34889 is depicted, plastidic is mentioned, or a mitochondrial transit peptide in case in Table I, application no. 19, in column 6 in the same line as SEQ ID NO. 34889 is depicted, mitochondric is mentioned; which is operable linked, too.

[100322] In another embodiment thereof the expression cassette does not comprise an additional transit peptide in case in Table I, application no. 19, in column 6 in the same line as SEQ ID NO. 34889 is depicted, non-targeted is mentioned.

[100323] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 103958, or a homolog or fragment thereof, or a homolog thereof being depicted in Table I, application no. 19, column 8, in the same line as SEQ ID NO. 103958, preferably the coding region thereof, a homolog or a fragment thereof; which are operable linked.

[100324] In an embodiment thereof the expression cassette comprises in addition c) a nucleic acid molecule encoding a transit peptide, especially a plastidal transit petide in case in Table I, application no. 19, in column 6 in the same line as SEQ ID NO. 103958 is depicted, plastidic is mentioned, or a mitochondrial transit peptide in case in Table I, application no. 19, in column 6 in the same line as SEQ ID NO. 103958 is depicted, mitochondric is mentioned; which is operable linked, too.

[100325] In another embodiment thereof the expression cassette does not comprise an additional transit peptide in case in Table I, application no. 19, in column 6 in the same line as SEQ ID NO. 103958 is depicted, non-targeted is mentioned.

[100326] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7081, or a homolog or fragment thereof, or a homolog thereof being depicted in Table I, application no. 19, column 8, in the same line as SEQ ID NO. 7081, preferably the coding region thereof, a homolog or a fragment thereof; which are operable linked.

[100327] In an embodiment thereof the expression cassette comprises in addition c) a nucleic acid molecule encoding a transit peptide, especially a plastidal transit petide in case in Table I, application no. 19, in column 6 in the same line as SEQ ID NO. 7081 is depicted, plastidic is mentioned, or a mitochondrial transit peptide in case in Table I, application no. 19, in column 6 in the same line as SEQ ID NO. 7081 is depicted, mitochondric is mentioned; which is operable linked, too.

[100328] In another embodiment thereof the expression cassette does not comprise an additional transit peptide in case in Table I, application no. 19, in column 6 in the same line as SEQ ID NO. 7081 is depicted, non-targeted is mentioned.

[100329] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7333, or a homolog or fragment thereof, or a homolog thereof being depicted in Table I, application no. 19, column 8, in the same line as SEQ ID NO. 7333, preferably the coding region thereof, a homolog or a fragment thereof; which are operable linked.

[100330] In an embodiment thereof the expression cassette comprises in addition c) a nucleic acid molecule encoding a transit peptide, especially a plastidal transit petide in case in Table I, application no. 19, in column 6 in the same line as SEQ ID NO. 7333 is depicted, plastidic is mentioned, or a mitochondrial transit peptide in case in Table I, application no. 19, in column 6 in the same line as SEQ ID NO. 7333 is depicted, mitochondric is mentioned; which is operable linked, too.

[100331] In another embodiment thereof the expression cassette does not comprise an additional transit peptide in case in Table I, application no. 19, in column 6 in the same line as SEQ ID NO. 7333 is depicted, non-targeted is mentioned.

[100332] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 35936, or a homolog or fragment thereof, or a homolog thereof being depicted in Table I, application no. 19, column 8, in the same line as SEQ ID NO. 35936, preferably the coding region thereof, a homolog or a fragment thereof; which are operable linked.

[100333] In an embodiment thereof the expression cassette comprises in addition c) a nucleic acid molecule encoding a transit peptide, especially a plastidal transit petide in case in Table I, application no. 19, in column 6 in the same line as SEQ ID NO. 35936 is depicted, plastidic is mentioned, or a mitochondrial transit peptide in case in Table I, application no. 19, in column 6 in the same line as SEQ ID NO. 35936 is depicted, mitochondric is mentioned; which is operable linked, too.

[100334] In another embodiment thereof the expression cassette does not comprise an additional transit peptide in case in Table I, application no. 19, in column 6 in the same line as SEQ ID NO. 35936 is depicted, non-targeted is mentioned.

[100335] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 152576, or a homolog or fragment thereof, or a homolog thereof being depicted in Table I, application no. 19, column 8, in the same line as SEQ ID NO. 152576, preferably the coding region thereof, a homolog or a fragment thereof; which are operable linked.

[100336] In an embodiment thereof the expression cassette comprises in addition c) a nucleic acid molecule encoding a transit peptide, especially a plastidal transit petide in case in Table I, application no. 19, in column 6 in the same line as SEQ ID NO. 152576 is depicted, plastidic is mentioned, or a mitochondrial transit peptide in case in Table I, application no. 19, in column 6 in the same line as SEQ ID NO. 152576 is depicted, mitochondric is mentioned; which is operable linked, too.

[100337] In another embodiment thereof the expression cassette does not comprise an additional transit peptide in case in Table I, application no. 19, in column 6 in the same line as SEQ ID NO. 152576 is depicted, non-targeted is mentioned.

[100338] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 131359, or a homolog or fragment thereof, or a homolog thereof being depicted in Table I, application no. 19, column 8, in the same line as SEQ ID NO. 131359, preferably the coding region thereof, a homolog or a fragment thereof; which are operable linked.

[100339] In an embodiment thereof the expression cassette comprises in addition c) a nucleic acid molecule encoding a transit peptide, especially a plastidal transit petide in case in Table I, application no. 19, in column 6 in the same line as SEQ ID NO. 131359 is depicted, plastidic is mentioned, or a mitochondrial transit peptide in case in Table I, application no. 19, in column 6 in the same line as SEQ ID NO. 131359 is depicted, mitochondric is mentioned; which is operable linked, too.

[100340] In another embodiment thereof the expression cassette does not comprise an additional transit peptide in case in Table I, application no. 19, in column 6 in the same line as SEQ ID NO. 131359 is depicted, non-targeted is mentioned.

[100341] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 36670, or a homolog or fragment thereof, or a homolog thereof being depicted in Table I, application no. 19, column 8, in the same line as SEQ ID NO. 36670, preferably the coding region thereof, a homolog or a fragment thereof; which are operable linked.

[100342] In an embodiment thereof the expression cassette comprises in addition c) a nucleic acid molecule encoding a transit peptide, especially a plastidal transit petide in case in Table I, application no. 19, in column 6 in the same line as SEQ ID NO. 36670 is depicted, plastidic is mentioned, or a mitochondrial transit peptide in case in Table I, application no. 19, in column 6 in the same line as SEQ ID NO. 36670 is depicted, mitochondric is mentioned; which is operable linked, too.

[100343] In another embodiment thereof the expression cassette does not comprise an additional transit peptide in case in Table I, application no. 19, in column 6 in the same line as SEQ ID NO. 36670 is depicted, non-targeted is mentioned.

[100344] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 144479, or a homolog or fragment thereof, or a homolog thereof being depicted in Table I, application no. 19, column 8, in the same line as SEQ ID NO. 144479, preferably the coding region thereof, a homolog or a fragment thereof; which are operable linked.

[100345] In an embodiment thereof the expression cassette comprises in addition c) a nucleic acid molecule encoding a transit peptide, especially a plastidal transit petide in case in Table I, application no. 19, in column 6 in the same line as SEQ ID NO. 144479 is depicted, plastidic is mentioned, or a mitochondrial transit peptide in case in Table I, application no. 19, in column 6 in the same line as SEQ ID NO. 144479 is depicted, mitochondric is mentioned; which is operable linked, too.

[100346] In another embodiment thereof the expression cassette does not comprise an additional transit peptide in case in Table I, application no. 19, in column 6 in the same line as SEQ ID NO. 144479 is depicted, non-targeted is mentioned.

[100347] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7941, or a homolog or fragment thereof, or a homolog thereof being depicted in Table I, application no. 19, column 8, in the same line as SEQ ID NO. 7941, preferably the coding region thereof, a homolog or a fragment thereof; which are operable linked.

[100348] In an embodiment thereof the expression cassette comprises in addition c) a nucleic acid molecule encoding a transit peptide, especially a plastidal transit petide in case in Table I, application no. 19, in column 6 in the same line as SEQ ID NO. 7941 is depicted, plastidic is mentioned, or a mitochondrial transit peptide in case in Table I, application no. 19, in column 6 in the same line as SEQ ID NO. 7941 is depicted, mitochondric is mentioned; which is operable linked, too.

[100349] In another embodiment thereof the expression cassette does not comprise an additional transit peptide in case in Table I, application no. 19, in column 6 in the same line as SEQ ID NO. 7941 is depicted, non-targeted is mentioned.

[100350] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 114552, or a homolog or fragment thereof, or a homolog thereof being depicted in Table I, application no. 19, column 8, in the same line as SEQ ID NO. 114552, preferably the coding region thereof, a homolog or a fragment thereof; which are operable linked.

[100351] In an embodiment thereof the expression cassette comprises in addition c) a nucleic acid molecule encoding a transit peptide, especially a plastidal transit petide in case in Table I, application no. 19, in column 6 in the same line as SEQ ID NO. 114552 is depicted, plastidic is mentioned, or a mitochondrial transit peptide in case in Table I, application no. 19, in column 6 in the same line as SEQ ID NO. 114552 is depicted, mitochondric is mentioned; which is operable linked, too.

[100352] In another embodiment thereof the expression cassette does not comprise an additional transit peptide in case in Table I, application no. 19, in column 6 in the same line as SEQ ID NO. 114552 is depicted, non-targeted is mentioned.

[100353] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 37400, or a homolog or fragment thereof, or a homolog thereof being depicted in Table I, application no. 19, column 8, in the same line as SEQ ID NO. 37400, preferably the coding region thereof, a homolog or a fragment thereof; which are operable linked.

[100354] In an embodiment thereof the expression cassette comprises in addition c) a nucleic acid molecule encoding a transit peptide, especially a plastidal transit petide in case in Table I, application no. 19, in column 6 in the same line as SEQ ID NO. 37400 is depicted, plastidic is mentioned, or a mitochondrial transit peptide in case in Table I, application no. 19, in column 6 in the same line as SEQ ID NO. 37400 is depicted, mitochondric is mentioned; which is operable linked, too.

[100355] In another embodiment thereof the expression cassette does not comprise an additional transit peptide in case in Table I, application no. 19, in column 6 in the same line as SEQ ID NO. 37400 is depicted, non-targeted is mentioned.

[100356] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 112111, or a homolog or fragment thereof, or a homolog thereof being depicted in Table I, application no. 19, column 8, in the same line as SEQ ID NO. 112111, preferably the coding region thereof, a homolog or a fragment thereof; which are operable linked.

[100357] In an embodiment thereof the expression cassette comprises in addition c) a nucleic acid molecule encoding a transit peptide, especially a plastidal transit petide in case in Table I, application no. 19, in column 6 in the same line as SEQ ID NO. 112111 is depicted, plastidic is mentioned, or a mitochondrial transit peptide in case in Table I, application no. 19, in column 6 in the same line as SEQ ID NO. 112111 is depicted, mitochondric is mentioned; which is operable linked, too.

[100358] In another embodiment thereof the expression cassette does not comprise an additional transit peptide in case in Table I, application no. 19, in column 6 in the same line as SEQ ID NO. 112111 is depicted, non-targeted is mentioned.

[100359] In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 98778, or a homolog or fragment thereof, or a homolog thereof being depicted in Table I, application no. 19, column 8, in the same line as SEQ ID NO. 98778, preferably the coding region thereof, a homolog or a fragment thereof; which are operable linked.

[100360] In an embodiment thereof the expression cassette comprises in addition c) a nucleic acid molecule encoding a transit peptide, especially a plastidal transit petide in case in Table I, application no. 19, in column 6 in the same line as SEQ ID NO. 98778 is depicted, plastidic is mentioned, or a mitochondrial transit peptide in case in Table I, application no. 19, in column 6 in the same line as SEQ ID NO. 98778 is depicted, mitochondric is mentioned; which is operable linked, too.

[100361] In another embodiment thereof the expression cassette does not comprise an additional transit peptide in case in Table I, application no. 19, in column 6 in the same line as SEQ ID NO. 98778 is depicted, non-targeted is mentioned.

[100362] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38226, or a homolog or fragment thereof, or a homolog thereof being depicted in Table I, application no. 19, column 8, in the same line as SEQ ID NO. 38226, preferably the coding region thereof, a homolog or a fragment thereof; which are operable linked.

[100363] In an embodiment thereof the expression cassette comprises in addition c) a nucleic acid molecule encoding a transit peptide, especially a plastidal transit petide in case in Table I, application no. 19, in column 6 in the same line as SEQ ID NO. 38226 is depicted, plastidic is mentioned, or a mitochondrial transit peptide in case in Table I, application no. 19, in column 6 in the same line as SEQ ID NO. 38226 is depicted, mitochondric is mentioned; which is operable linked, too.

[100364] In another embodiment thereof the expression cassette does not comprise an additional transit peptide in case in Table I, application no. 19, in column 6 in the same line as SEQ ID NO. 38226 is depicted, non-targeted is mentioned.

[100365] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 76890, or a homolog or fragment thereof, or a homolog thereof being depicted in Table I, application no. 19, column 8, in the same line as SEQ ID NO. 76890, preferably the coding region thereof, a homolog or a fragment thereof; which are operable linked.

[100366] In an embodiment thereof the expression cassette comprises in addition c) a nucleic acid molecule encoding a transit peptide, especially a plastidal transit petide in case in Table I, application no. 19, in column 6 in the same line as SEQ ID NO. 76890 is depicted, plastidic is mentioned, or a mitochondrial transit peptide in case in Table I, application no. 19, in column 6 in the same line as SEQ ID NO. 76890 is depicted, mitochondric is mentioned; which is operable linked, too.

[100367] In another embodiment thereof the expression cassette does not comprise an additional transit peptide in case in Table I, application no. 19, in column 6 in the same line as SEQ ID NO. 76890 is depicted, non-targeted is mentioned.

[100368] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 131489, or a homolog or fragment thereof, or a homolog thereof being depicted in Table I, application no. 19, column 8, in the same line as SEQ ID NO. 131489, preferably the coding region thereof, a homolog or a fragment thereof; which are operable linked.

[100369] In an embodiment thereof the expression cassette comprises in addition c) a nucleic acid molecule encoding a transit peptide, especially a plastidal transit petide in case in Table I, application no. 19, in column 6 in the same line as SEQ ID NO. 131489 is depicted, plastidic is mentioned, or a mitochondrial transit peptide in case in Table I, application no. 19, in column 6 in the same line as SEQ ID NO. 131489 is depicted, mitochondric is mentioned; which is operable linked, too.

[100370] In another embodiment thereof the expression cassette does not comprise an additional transit peptide in case in Table I, application no. 19, in column 6 in the same line as SEQ ID NO. 131489 is depicted, non-targeted is mentioned.

[100371] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38266, or a homolog or fragment thereof, or a homolog thereof being depicted in Table I, application no. 19, column 8, in the same line as SEQ ID NO. 38266, preferably the coding region thereof, a homolog or a fragment thereof; which are operable linked.

[100372] In an embodiment thereof the expression cassette comprises in addition c) a nucleic acid molecule encoding a transit peptide, especially a plastidal transit petide in case in Table I, application no. 19, in column 6 in the same line as SEQ ID NO. 38266 is depicted, plastidic is mentioned, or a mitochondrial transit peptide in case in Table I, application no. 19, in column 6 in the same line as SEQ ID NO. 38266 is depicted, mitochondric is mentioned; which is operable linked, too.

[100373] In another embodiment thereof the expression cassette does not comprise an additional transit peptide in case in Table I, application no. 19, in column 6 in the same line as SEQ ID NO. 38266 is depicted, non-targeted is mentioned.

[100374] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7947, or a homolog or fragment thereof, or a homolog thereof being depicted in Table I, application no. 19, column 8, in the same line as SEQ ID NO. 7947, preferably the coding region thereof, a homolog or a fragment thereof; which are operable linked.

[100375] In an embodiment thereof the expression cassette comprises in addition c) a nucleic acid molecule encoding a transit peptide, especially a plastidal transit petide in case in Table I, application no. 19, in column 6 in the same line as SEQ ID NO. 7947 is depicted, plastidic is mentioned, or a mitochondrial transit peptide in case in Table I, application no. 19, in column 6 in the same line as SEQ ID NO. 7947 is depicted, mitochondric is mentioned; which is operable linked, too.

[100376] In another embodiment thereof the expression cassette does not comprise an additional transit peptide in case in Table I, application no. 19, in column 6 in the same line as SEQ ID NO. 7947 is depicted, non-targeted is mentioned.

[100377] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 152991, or a homolog or fragment thereof, or a homolog thereof being depicted in Table I, application no. 19, column 8, in the same line as SEQ ID NO. 152991, preferably the coding region thereof, a homolog or a fragment thereof; which are operable linked.

[100378] In an embodiment thereof the expression cassette comprises in addition c) a nucleic acid molecule encoding a transit peptide, especially a plastidal transit petide in case in Table I, application no. 19, in column 6 in the same line as SEQ ID NO. 152991 is depicted, plastidic is mentioned, or a mitochondrial transit peptide in case in Table I, application no. 19, in column 6 in the same line as SEQ ID NO. 152991 is depicted, mitochondric is mentioned; which is operable linked, too.

[100379] In another embodiment thereof the expression cassette does not comprise an additional transit peptide in case in Table I, application no. 19, in column 6 in the same line as SEQ ID NO. 152991 is depicted, non-targeted is mentioned.

[100380] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 145142, or a homolog or fragment thereof, or a homolog thereof being depicted in Table I, application no. 19, column 8, in the same line as SEQ ID NO. 145142, preferably the coding region thereof, a homolog or a fragment thereof; which are operable linked.

[100381] In an embodiment thereof the expression cassette comprises in addition c) a nucleic acid molecule encoding a transit peptide, especially a plastidal transit petide in case in Table I, application no. 19, in column 6 in the same line as SEQ ID NO. 145142 is depicted, plastidic is mentioned, or a mitochondrial transit peptide in case in Table I, application no. 19, in column 6 in the same line as SEQ ID NO. 145142 is depicted, mitochondric is mentioned; which is operable linked, too.

[100382] In another embodiment thereof the expression cassette does not comprise an additional transit peptide in case in Table I, application no. 19, in column 6 in the same line as SEQ ID NO. 145142 is depicted, non-targeted is mentioned.

[100383] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78138, or a homolog or fragment thereof, or a homolog thereof being depicted in Table I, application no. 19, column 8, in the same line as SEQ ID NO. 78138, preferably the coding region thereof, a homolog or a fragment thereof; which are operable linked.

[100384] In an embodiment thereof the expression cassette comprises in addition c) a nucleic acid molecule encoding a transit peptide, especially a plastidal transit petide in case in Table I, application no. 19, in column 6 in the same line as SEQ ID NO. 78138 is depicted, plastidic is mentioned, or a mitochondrial transit peptide in case in Table I, application no. 19, in column 6 in the same line as SEQ ID NO. 78138 is depicted, mitochondric is mentioned; which is operable linked, too.

[100385] In another embodiment thereof the expression cassette does not comprise an additional transit peptide in case in Table I, application no. 19, in column 6 in the same line as SEQ ID NO. 78138 is depicted, non-targeted is mentioned.

[100386] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78154, or a homolog or fragment thereof, or a homolog thereof being depicted in Table I, application no. 19, column 8, in the same line as SEQ ID NO. 78154, preferably the coding region thereof, a homolog or a fragment thereof; which are operable linked.

[100387] In an embodiment thereof the expression cassette comprises in addition c) a nucleic acid molecule encoding a transit peptide, especially a plastidal transit petide in case in Table I, application no. 19, in column 6 in the same line as SEQ ID NO. 78154 is depicted, plastidic is mentioned, or a mitochondrial transit peptide in case in Table I, application no. 19, in column 6 in the same line as SEQ ID NO. 78154 is depicted, mitochondric is mentioned; which is operable linked, too.

[100388] In another embodiment thereof the expression cassette does not comprise an additional transit peptide in case in Table I, application no. 19, in column 6 in the same line as SEQ ID NO. 78154 is depicted, non-targeted is mentioned.

[100389] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38300, or a homolog or fragment thereof, or a homolog thereof being depicted in Table I, application no. 19, column 8, in the same line as SEQ ID NO. 38300, preferably the coding region thereof, a homolog or a fragment thereof; which are operable linked.

[100390] In an embodiment thereof the expression cassette comprises in addition c) a nucleic acid molecule encoding a transit peptide, especially a plastidal transit petide in case in Table I, application no. 19, in column 6 in the same line as SEQ ID NO. 38300 is depicted, plastidic is mentioned, or a mitochondrial transit peptide in case in Table I, application no. 19, in column 6 in the same line as SEQ ID NO. 38300 is depicted, mitochondric is mentioned; which is operable linked, too.

[100391] In another embodiment thereof the expression cassette does not comprise an additional transit peptide in case in Table I, application no. 19, in column 6 in the same line as SEQ ID NO. 38300 is depicted, non-targeted is mentioned.

[100392] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78753, or a homolog or fragment thereof, or a homolog thereof being depicted in Table I, application no. 19, column 8, in the same line as SEQ ID NO. 78753, preferably the coding region thereof, a homolog or a fragment thereof; which are operable linked.

[100393] In an embodiment thereof the expression cassette comprises in addition c) a nucleic acid molecule encoding a transit peptide, especially a plastidal transit petide in case in Table I, application no. 19, in column 6 in the same line as SEQ ID NO. 78753 is depicted, plastidic is mentioned, or a mitochondrial transit peptide in case in Table I, application no. 19, in column 6 in the same line as SEQ ID NO. 78753 is depicted, mitochondric is mentioned; which is operable linked, too.

[100394] In another embodiment thereof the expression cassette does not comprise an additional transit peptide in case in Table I, application no. 19, in column 6 in the same line as SEQ ID NO. 78753 is depicted, non-targeted is mentioned.

[100395] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78771, or a homolog or fragment thereof, or a homolog thereof being depicted in Table I, application no. 19, column 8, in the same line as SEQ ID NO. 78771, preferably the coding region thereof, a homolog or a fragment thereof; which are operable linked.

[100396] In an embodiment thereof the expression cassette comprises in addition c) a nucleic acid molecule encoding a transit peptide, especially a plastidal transit petide in case in Table I, application no. 19, in column 6 in the same line as SEQ ID NO. 78771 is depicted, plastidic is mentioned, or a mitochondrial transit peptide in case in Table I, application no. 19, in column 6 in the same line as SEQ ID NO. 78771 is depicted, mitochondric is mentioned; which is operable linked, too.

[100397] In another embodiment thereof the expression cassette does not comprise an additional transit peptide in case in Table I, application no. 19, in column 6 in the same line as SEQ ID NO. 78771 is depicted, non-targeted is mentioned.

[100398] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 124907, or a homolog or fragment thereof, or a homolog thereof being depicted in Table I, application no. 19, column 8, in the same line as SEQ ID NO. 124907, preferably the coding region thereof, a homolog or a fragment thereof; which are operable linked.

[100399] In an embodiment thereof the expression cassette comprises in addition c) a nucleic acid molecule encoding a transit peptide, especially a plastidal transit petide in case in Table I, application no. 19, in column 6 in the same line as SEQ ID NO. 124907 is depicted, plastidic is mentioned, or a mitochondrial transit peptide in case in Table I, application no. 19, in column 6 in the same line as SEQ ID NO. 124907 is depicted, mitochondric is mentioned; which is operable linked, too.

[100400] In another embodiment thereof the expression cassette does not comprise an additional transit peptide in case in Table I, application no. 19, in column 6 in the same line as SEQ ID NO. 124907 is depicted, non-targeted is mentioned.

[100401] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 153072, or a homolog or fragment thereof, or a homolog thereof being depicted in Table I, application no. 19, column 8, in the same line as SEQ ID NO. 153072, preferably the coding region thereof, a homolog or a fragment thereof; which are operable linked.

[100402] In an embodiment thereof the expression cassette comprises in addition c) a nucleic acid molecule encoding a transit peptide, especially a plastidal transit petide in case in Table I, application no. 19, in column 6 in the same line as SEQ ID NO. 153072 is depicted, plastidic is mentioned, or a mitochondrial transit peptide in case in Table I, application no. 19, in column 6 in the same line as SEQ ID NO. 153072 is depicted, mitochondric is mentioned; which is operable linked, too.

[100403] In another embodiment thereof the expression cassette does not comprise an additional transit peptide in case in Table I, application no. 19, in column 6 in the same line as SEQ ID NO. 153072 is depicted, non-targeted is mentioned.

[100404] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 131539, or a homolog or fragment thereof, or a homolog thereof being depicted in Table I, application no. 19, column 8, in the same line as SEQ ID NO. 131539, preferably the coding region thereof, a homolog or a fragment thereof; which are operable linked.

[100405] In an embodiment thereof the expression cassette comprises in addition c) a nucleic acid molecule encoding a transit peptide, especially a plastidal transit petide in case in Table I, application no. 19, in column 6 in the same line as SEQ ID NO. 131539 is depicted, plastidic is mentioned, or a mitochondrial transit peptide in case in Table I, application no. 19, in column 6 in the same line as SEQ ID NO. 131539 is depicted, mitochondric is mentioned; which is operable linked, too.

[100406] In another embodiment thereof the expression cassette does not comprise an additional transit peptide in case in Table I, application no. 19, in column 6 in the same line as SEQ ID NO. 131539 is depicted, non-targeted is mentioned.

[100407] In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 8033, or a homolog or fragment thereof, or a homolog thereof being depicted in Table I, application no. 19, column 8, in the same line as SEQ ID NO. 8033, preferably the coding region thereof, a homolog or a fragment thereof; which are operable linked.

[100408] In an embodiment thereof the expression cassette comprises in addition c) a nucleic acid molecule encoding a transit peptide, especially a plastidal transit petide in case in Table I, application no. 19, in column 6 in the same line as SEQ ID NO. 8033 is depicted, plastidic is mentioned, or a mitochondrial transit peptide in case in Table I, application no. 19, in column 6 in the same line as SEQ ID NO. 8033 is depicted, mitochondric is mentioned; which is operable linked, too.

[100409] In another embodiment thereof the expression cassette does not comprise an additional transit peptide in case in Table I, application no. 19, in column 6 in the same line as SEQ ID NO. 8033 is depicted, non-targeted is mentioned.

[100410] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78953, or a homolog or fragment thereof, or a homolog thereof being depicted in Table I, application no. 19, column 8, in the same line as SEQ ID NO. 78953, preferably the coding region thereof, a homolog or a fragment thereof; which are operable linked.

[100411] In an embodiment thereof the expression cassette comprises in addition c) a nucleic acid molecule encoding a transit peptide, especially a plastidal transit petide in case in Table I, application no. 19, in column 6 in the same line as SEQ ID NO. 78953 is depicted, plastidic is mentioned, or a mitochondrial transit peptide in case in Table I, application no. 19, in column 6 in the same line as SEQ ID NO. 78953 is depicted, mitochondric is mentioned; which is operable linked, too.

[100412] In another embodiment thereof the expression cassette does not comprise an additional transit peptide in case in Table I, application no. 19, in column 6 in the same line as SEQ ID NO. 78953 is depicted, non-targeted is mentioned.

[100413] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 79056, or a homolog or fragment thereof, or a homolog thereof being depicted in Table I, application no. 19, column 8, in the same line as SEQ ID NO. 79056, preferably the coding region thereof, a homolog or a fragment thereof; which are operable linked.

[100414] In an embodiment thereof the expression cassette comprises in addition c) a nucleic acid molecule encoding a transit peptide, especially a plastidal transit petide in case in Table I, application no. 19, in column 6 in the same line as SEQ ID NO. 79056 is depicted, plastidic is mentioned, or a mitochondrial transit peptide in case in Table I, application no. 19, in column 6 in the same line as SEQ ID NO. 79056 is depicted, mitochondric is mentioned; which is operable linked, too.

[100415] In another embodiment thereof the expression cassette does not comprise an additional transit peptide in case in Table I, application no. 19, in column 6 in the same line as SEQ ID NO. 79056 is depicted, non-targeted is mentioned.

[100416] In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 99371, or a homolog or fragment thereof, or a homolog thereof being depicted in Table I, application no. 19, column 8, in the same line as SEQ ID NO. 99371, preferably the coding region thereof, a homolog or a fragment thereof; which are operable linked.

[100417] In an embodiment thereof the expression cassette comprises in addition c) a nucleic acid molecule encoding a transit peptide, especially a plastidal transit petide in case in Table I, application no. 19, in column 6 in the same line as SEQ ID NO. 99371 is depicted, plastidic is mentioned, or a mitochondrial transit peptide in case in Table I, application no. 19, in column 6 in the same line as SEQ ID NO. 99371 is depicted, mitochondric is mentioned; which is operable linked, too.

[100418] In another embodiment thereof the expression cassette does not comprise an additional transit peptide in case in Table I, application no. 19, in column 6 in the same line as SEQ ID NO. 99371 is depicted, non-targeted is mentioned.

[100419] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 8920, or a homolog or fragment thereof, or a homolog thereof being depicted in Table I, application no. 19, column 8, in the same line as SEQ ID NO. 8920, preferably the coding region thereof, a homolog or a fragment thereof; which are operable linked.

[100420] In an embodiment thereof the expression cassette comprises in addition c) a nucleic acid molecule encoding a transit peptide, especially a plastidal transit petide in case in Table I, application no. 19, in column 6 in the same line as SEQ ID NO. 8920 is depicted, plastidic is mentioned, or a mitochondrial transit peptide in case in Table I, application no. 19, in column 6 in the same line as SEQ ID NO. 8920 is depicted, mitochondric is mentioned; which is operable linked, too.

[100421] In another embodiment thereof the expression cassette does not comprise an additional transit peptide in case in Table I, application no. 19, in column 6 in the same line as SEQ ID NO. 8920 is depicted, non-targeted is mentioned.

[100422] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115595, or a homolog or fragment thereof, or a homolog thereof being depicted in Table I, application no. 19, column 8, in the same line as SEQ ID NO. 115595, preferably the coding region thereof, a homolog or a fragment thereof; which are operable linked.

[100423] In an embodiment thereof the expression cassette comprises in addition c) a nucleic acid molecule encoding a transit peptide, especially a plastidal transit petide in case in Table I, application no. 19, in column 6 in the same line as SEQ ID NO. 115595 is depicted, plastidic is mentioned, or a mitochondrial transit peptide in case in Table I, application no. 19, in column 6 in the same line as SEQ ID NO. 115595 is depicted, mitochondric is mentioned; which is operable linked, too.

[100424] In another embodiment thereof the expression cassette does not comprise an additional transit peptide in case in Table I, application no. 19, in column 6 in the same line as SEQ ID NO. 115595 is depicted, non-targeted is mentioned.

[100425] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 125085, or a homolog or fragment thereof, or a homolog thereof being depicted in Table I, application no. 19, column 8, in the same line as SEQ ID NO. 125085, preferably the coding region thereof, a homolog or a fragment thereof; which are operable linked.

[100426] In an embodiment thereof the expression cassette comprises in addition c) a nucleic acid molecule encoding a transit peptide, especially a plastidal transit petide in case in Table I, application no. 19, in column 6 in the same line as SEQ ID NO. 125085 is depicted, plastidic is mentioned, or a mitochondrial transit peptide in case in Table I, application no. 19, in column 6 in the same line as SEQ ID NO. 125085 is depicted, mitochondric is mentioned; which is operable linked, too.

[100427] In another embodiment thereof the expression cassette does not comprise an additional transit peptide in case in Table I, application no. 19, in column 6 in the same line as SEQ ID NO. 125085 is depicted, non-targeted is mentioned.

[100428] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39040, or a homolog or fragment thereof, or a homolog thereof being depicted in Table I, application no. 19, column 8, in the same line as SEQ ID NO. 39040, preferably the coding region thereof, a homolog or a fragment thereof; which are operable linked.

[100429] In an embodiment thereof the expression cassette comprises in addition c) a nucleic acid molecule encoding a transit peptide, especially a plastidal transit petide in case in Table I, application no. 19, in column 6 in the same line as SEQ ID NO. 39040 is depicted, plastidic is mentioned, or a mitochondrial transit peptide in case in Table I, application no. 19, in column 6 in the same line as SEQ ID NO. 39040 is depicted, mitochondric is mentioned; which is operable linked, too.

[100430] In another embodiment thereof the expression cassette does not comprise an additional transit peptide in case in Table I, application no. 19, in column 6 in the same line as SEQ ID NO. 39040 is depicted, non-targeted is mentioned.

[100431] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 146238, or a homolog or fragment thereof, or a homolog thereof being depicted in Table I, application no. 19, column 8, in the same line as SEQ ID NO. 146238, preferably the coding region thereof, a homolog or a fragment thereof; which are operable linked.

[100432] In an embodiment thereof the expression cassette comprises in addition c) a nucleic acid molecule encoding a transit peptide, especially a plastidal transit petide in case in Table I, application no. 19, in column 6 in the same line as SEQ ID NO. 146238 is depicted, plastidic is mentioned, or a mitochondrial transit peptide in case in Table I, application no. 19, in column 6 in the same line as SEQ ID NO. 146238 is depicted, mitochondric is mentioned; which is operable linked, too.

[100433] In another embodiment thereof the expression cassette does not comprise an additional transit peptide in case in Table I, application no. 19, in column 6 in the same line as SEQ ID NO. 146238 is depicted, non-targeted is mentioned.

[100434] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39237, or a homolog or fragment thereof, or a homolog thereof being depicted in Table I, application no. 19, column 8, in the same line as SEQ ID NO. 39237, preferably the coding region thereof, a homolog or a fragment thereof; which are operable linked.

[100435] In an embodiment thereof the expression cassette comprises in addition c) a nucleic acid molecule encoding a transit peptide, especially a plastidal transit petide in case in Table I, application no. 19, in column 6 in the same line as SEQ ID NO. 39237 is depicted, plastidic is mentioned, or a mitochondrial transit peptide in case in Table I, application no. 19, in column 6 in the same line as SEQ ID NO. 39237 is depicted, mitochondric is mentioned; which is operable linked, too.

[100436] In another embodiment thereof the expression cassette does not comprise an additional transit peptide in case in Table I, application no. 19, in column 6 in the same line as SEQ ID NO. 39237 is depicted, non-targeted is mentioned.

[100437] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115628, or a homolog or fragment thereof, or a homolog thereof being depicted in Table I, application no. 19, column 8, in the same line as SEQ ID NO. 115628, preferably the coding region thereof, a homolog or a fragment thereof; which are operable linked.

[100438] In an embodiment thereof the expression cassette comprises in addition c) a nucleic acid molecule encoding a transit peptide, especially a plastidal transit petide in case in Table I, application no. 19, in column 6 in the same line as SEQ ID NO. 115628 is depicted, plastidic is mentioned, or a mitochondrial transit peptide in case in Table I, application no. 19, in column 6 in the same line as SEQ ID NO. 115628 is depicted, mitochondric is mentioned; which is operable linked, too.

[100439] In another embodiment thereof the expression cassette does not comprise an additional transit peptide in case in Table I, application no. 19, in column 6 in the same line as SEQ ID NO. 115628 is depicted, non-targeted is mentioned.

[100440] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40299, or a homolog or fragment thereof, or a homolog thereof being depicted in Table I, application no. 19, column 8, in the same line as SEQ ID NO. 40299, preferably the coding region thereof, a homolog or a fragment thereof; which are operable linked.

[100441] In an embodiment thereof the expression cassette comprises in addition c) a nucleic acid molecule encoding a transit peptide, especially a plastidal transit petide in case in Table I, application no. 19, in column 6 in the same line as SEQ ID NO. 40299 is depicted, plastidic is mentioned, or a mitochondrial transit peptide in case in Table I, application no. 19, in column 6 in the same line as SEQ ID NO. 40299 is depicted, mitochondric is mentioned; which is operable linked, too.

[100442] In another embodiment thereof the expression cassette does not comprise an additional transit peptide in case in Table I, application no. 19, in column 6 in the same line as SEQ ID NO. 40299 is depicted, non-targeted is mentioned.

[100443] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40329, or a homolog or fragment thereof, or a homolog thereof being depicted in Table I, application no. 19, column 8, in the same line as SEQ ID NO. 40329, preferably the coding region thereof, a homolog or a fragment thereof; which are operable linked.

[100444] In an embodiment thereof the expression cassette comprises in addition c) a nucleic acid molecule encoding a transit peptide, especially a plastidal transit petide in case in Table I, application no. 19, in column 6 in the same line as SEQ ID NO. 40329 is depicted, plastidic is mentioned, or a mitochondrial transit peptide in case in Table I, application no. 19, in column 6 in the same line as SEQ ID NO. 40329 is depicted, mitochondric is mentioned; which is operable linked, too.

[100445] In another embodiment thereof the expression cassette does not comprise an additional transit peptide in case in Table I, application no. 19, in column 6 in the same line as SEQ ID NO. 40329 is depicted, non-targeted is mentioned.

[100446] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 99898, or a homolog or fragment thereof, or a homolog thereof being depicted in Table I, application no. 19, column 8, in the same line as SEQ ID NO. 99898, preferably the coding region thereof, a homolog or a fragment thereof; which are operable linked.

[100447] In an embodiment thereof the expression cassette comprises in addition c) a nucleic acid molecule encoding a transit peptide, especially a plastidal transit petide in case in Table I, application no. 19, in column 6 in the same line as SEQ ID NO. 99898 is depicted, plastidic is mentioned, or a mitochondrial transit peptide in case in Table I, application no. 19, in column 6 in the same line as SEQ ID NO. 99898 is depicted, mitochondric is mentioned; which is operable linked, too.

[100448] In another embodiment thereof the expression cassette does not comprise an additional transit peptide in case in Table I, application no. 19, in column 6 in the same line as SEQ ID NO. 99898 is depicted, non-targeted is mentioned.

[100449] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40665, or a homolog or fragment thereof, or a homolog thereof being depicted in Table I, application no. 19, column 8, in the same line as SEQ ID NO. 40665, preferably the coding region thereof, a homolog or a fragment thereof; which are operable linked.

[100450] In an embodiment thereof the expression cassette comprises in addition c) a nucleic acid molecule encoding a transit peptide, especially a plastidal transit petide in case in Table I, application no. 19, in column 6 in the same line as SEQ ID NO. 40665 is depicted, plastidic is mentioned, or a mitochondrial transit peptide in case in Table I, application no. 19, in column 6 in the same line as SEQ ID NO. 40665 is depicted, mitochondric is mentioned; which is operable linked, too.

[100451] In another embodiment thereof the expression cassette does not comprise an additional transit peptide in case in Table I, application no. 19, in column 6 in the same line as SEQ ID NO. 40665 is depicted, non-targeted is mentioned.

[100452] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40726, or a homolog or fragment thereof, or a homolog thereof being depicted in Table I, application no. 19, column 8, in the same line as SEQ ID NO. 40726, preferably the coding region thereof, a homolog or a fragment thereof; which are operable linked.

[100453] In an embodiment thereof the expression cassette comprises in addition c) a nucleic acid molecule encoding a transit peptide, especially a plastidal transit petide in case in Table I, application no. 19, in column 6 in the same line as SEQ ID NO. 40726 is depicted, plastidic is mentioned, or a mitochondrial transit peptide in case in Table I, application no. 19, in column 6 in the same line as SEQ ID NO. 40726 is depicted, mitochondric is mentioned; which is operable linked, too.

[100454] In another embodiment thereof the expression cassette does not comprise an additional transit peptide in case in Table I, application no. 19, in column 6 in the same line as SEQ ID NO. 40726 is depicted, non-targeted is mentioned.

[100455] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 9244, or a homolog or fragment thereof, or a homolog thereof being depicted in Table I, application no. 19, column 8, in the same line as SEQ ID NO. 9244, preferably the coding region thereof, a homolog or a fragment thereof; which are operable linked.

[100456] In an embodiment thereof the expression cassette comprises in addition c) a nucleic acid molecule encoding a transit peptide, especially a plastidal transit petide in case in Table I, application no. 19, in column 6 in the same line as SEQ ID NO. 9244 is depicted, plastidic is mentioned, or a mitochondrial transit peptide in case in Table I, application no. 19, in column 6 in the same line as SEQ ID NO. 9244 is depicted, mitochondric is mentioned; which is operable linked, too.

[100457] In another embodiment thereof the expression cassette does not comprise an additional transit peptide in case in Table I, application no. 19, in column 6 in the same line as SEQ ID NO. 9244 is depicted, non-targeted is mentioned.

[100458] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 80756, or a homolog or fragment thereof, or a homolog thereof being depicted in Table I, application no. 19, column 8, in the same line as SEQ ID NO. 80756, preferably the coding region thereof, a homolog or a fragment thereof; which are operable linked.

[100459] In an embodiment thereof the expression cassette comprises in addition c) a nucleic acid molecule encoding a transit peptide, especially a plastidal transit petide in case in Table I, application no. 19, in column 6 in the same line as SEQ ID NO. 80756 is depicted, plastidic is mentioned, or a mitochondrial transit peptide in case in Table I, application no. 19, in column 6 in the same line as SEQ ID NO. 80756 is depicted, mitochondric is mentioned; which is operable linked, too.

[100460] In another embodiment thereof the expression cassette does not comprise an additional transit peptide in case in Table I, application no. 19, in column 6 in the same line as SEQ ID NO. 80756 is depicted, non-targeted is mentioned.

[100461] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 40741, or a homolog or fragment thereof, or a homolog thereof being depicted in Table I, application no. 19, column 8, in the same line as SEQ ID NO. 40741, preferably the coding region thereof, a homolog or a fragment thereof; which are operable linked.

[100462] In an embodiment thereof the expression cassette comprises in addition c) a nucleic acid molecule encoding a transit peptide, especially a plastidal transit petide in case in Table I, application no. 19, in column 6 in the same line as SEQ ID NO. 40741 is depicted, plastidic is mentioned, or a mitochondrial transit peptide in case in Table I, application no. 19, in column 6 in the same line as SEQ ID NO. 40741 is depicted, mitochondric is mentioned; which is operable linked, too.

[100463] In another embodiment thereof the expression cassette does not comprise an additional transit peptide in case in Table I, application no. 19, in column 6 in the same line as SEQ ID NO. 40741 is depicted, non-targeted is mentioned.

[100464] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 81075, or a homolog or fragment thereof, or a homolog thereof being depicted in Table I, application no. 19, column 8, in the same line as SEQ ID NO. 81075, preferably the coding region thereof, a homolog or a fragment thereof; which are operable linked.

[100465] In an embodiment thereof the expression cassette comprises in addition c) a nucleic acid molecule encoding a transit peptide, especially a plastidal transit petide in case in Table I, application no. 19, in column 6 in the same line as SEQ ID NO. 81075 is depicted, plastidic is mentioned, or a mitochondrial transit peptide in case in Table I, application no. 19, in column 6 in the same line as SEQ ID NO. 81075 is depicted, mitochondric is mentioned; which is operable linked, too.

[100466] In another embodiment thereof the expression cassette does not comprise an additional transit peptide in case in Table I, application no. 19, in column 6 in the same line as SEQ ID NO. 81075 is depicted, non-targeted is mentioned.

[100467] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 81576, or a homolog or fragment thereof, or a homolog thereof being depicted in Table I, application no. 19, column 8, in the same line as SEQ ID NO. 81576, preferably the coding region thereof, a homolog or a fragment thereof; which are operable linked.

[100468] In an embodiment thereof the expression cassette comprises in addition c) a nucleic acid molecule encoding a transit peptide, especially a plastidal transit petide in case in Table I, application no. 19, in column 6 in the same line as SEQ ID NO. 81576 is depicted, plastidic is mentioned, or a mitochondrial transit peptide in case in Table I, application no. 19, in column 6 in the same line as SEQ ID NO. 81576 is depicted, mitochondric is mentioned; which is operable linked, too.

[100469] In another embodiment thereof the expression cassette does not comprise an additional transit peptide in case in Table I, application no. 19, in column 6 in the same line as SEQ ID NO. 81576 is depicted, non-targeted is mentioned.

[100470] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 82424, or a homolog or fragment thereof, or a homolog thereof being depicted in Table I, application no. 19, column 8, in the same line as SEQ ID NO. 82424, preferably the coding region thereof, a homolog or a fragment thereof; which are operable linked.

[100471] In an embodiment thereof the expression cassette comprises in addition c) a nucleic acid molecule encoding a transit peptide, especially a plastidal transit petide in case in Table I, application no. 19, in column 6 in the same line as SEQ ID NO. 82424 is depicted, plastidic is mentioned, or a mitochondrial transit peptide in case in Table I, application no. 19, in column 6 in the same line as SEQ ID NO. 82424 is depicted, mitochondric is mentioned; which is operable linked, too.

[100472] In another embodiment thereof the expression cassette does not comprise an additional transit peptide in case in Table I, application no. 19, in column 6 in the same line as SEQ ID NO. 82424 is depicted, non-targeted is mentioned.

[100473] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 153246, or a homolog or fragment thereof, or a homolog thereof being depicted in Table I, application no. 19, column 8, in the same line as SEQ ID NO. 153246, preferably the coding region thereof, a homolog or a fragment thereof; which are operable linked.

[100474] In an embodiment thereof the expression cassette comprises in addition c) a nucleic acid molecule encoding a transit peptide, especially a plastidal transit petide in case in Table I, application no. 19, in column 6 in the same line as SEQ ID NO. 153246 is depicted, plastidic is mentioned, or a mitochondrial transit peptide in case in Table I, application no. 19, in column 6 in the same line as SEQ ID NO. 153246 is depicted, mitochondric is mentioned; which is operable linked, too.

[100475] In another embodiment thereof the expression cassette does not comprise an additional transit peptide in case in Table I, application no. 19, in column 6 in the same line as SEQ ID NO. 153246 is depicted, non-targeted is mentioned.

[100476] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 153395, or a homolog or fragment thereof, or a homolog thereof being depicted in Table I, application no. 19, column 8, in the same line as SEQ ID NO. 153395, preferably the coding region thereof, a homolog or a fragment thereof; which are operable linked.

[100477] In an embodiment thereof the expression cassette comprises in addition c) a nucleic acid molecule encoding a transit peptide, especially a plastidal transit petide in case in Table I, application no. 19, in column 6 in the same line as SEQ ID NO. 153395 is depicted, plastidic is mentioned, or a mitochondrial transit peptide in case in Table I, application no. 19, in column 6 in the same line as SEQ ID NO. 153395 is depicted, mitochondric is mentioned; which is operable linked, too.

[100478] In another embodiment thereof the expression cassette does not comprise an additional transit peptide in case in Table I, application no. 19, in column 6 in the same line as SEQ ID NO. 153395 is depicted, non-targeted is mentioned.

[100479] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 153618, or a homolog or fragment thereof, or a homolog thereof being depicted in Table I, application no. 19, column 8, in the same line as SEQ ID NO. 153618, preferably the coding region thereof, a homolog or a fragment thereof; which are operable linked.

[100480] In an embodiment thereof the expression cassette comprises in addition c) a nucleic acid molecule encoding a transit peptide, especially a plastidal transit petide in case in Table I, application no. 19, in column 6 in the same line as SEQ ID NO. 153618 is depicted, plastidic is mentioned, or a mitochondrial transit peptide in case in Table I, application no. 19, in column 6 in the same line as SEQ ID NO. 153618 is depicted, mitochondric is mentioned; which is operable linked, too.

[100481] In another embodiment thereof the expression cassette does not comprise an additional transit peptide in case in Table I, application no. 19, in column 6 in the same line as SEQ ID NO. 153618 is depicted, non-targeted is mentioned.

[100482] In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 9492, or a homolog or fragment thereof, or a homolog thereof being depicted in Table I, application no. 19, column 8, in the same line as SEQ ID NO. 9492, preferably the coding region thereof, a homolog or a fragment thereof; which are operable linked.

[100483] In an embodiment thereof the expression cassette comprises in addition c) a nucleic acid molecule encoding a transit peptide, especially a plastidal transit petide in case in Table I, application no. 19, in column 6 in the same line as SEQ ID NO. 9492 is depicted, plastidic is mentioned, or a mitochondrial transit peptide in case in Table I, application no. 19, in column 6 in the same line as SEQ ID NO. 9492 is depicted, mitochondric is mentioned; which is operable linked, too.

[100484] In another embodiment thereof the expression cassette does not comprise an additional transit peptide in case in Table I, application no. 19, in column 6 in the same line as SEQ ID NO. 9492 is depicted, non-targeted is mentioned.

[100485] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 42502, or a homolog or fragment thereof, or a homolog thereof being depicted in Table I, application no. 19, column 8, in the same line as SEQ ID NO. 42502, preferably the coding region thereof, a homolog or a fragment thereof; which are operable linked.

[100486] In an embodiment thereof the expression cassette comprises in addition c) a nucleic acid molecule encoding a transit peptide, especially a plastidal transit petide in case in Table I, application no. 19, in column 6 in the same line as SEQ ID NO. 42502 is depicted, plastidic is mentioned, or a mitochondrial transit peptide in case in Table I, application no. 19, in column 6 in the same line as SEQ ID NO. 42502 is depicted, mitochondric is mentioned; which is operable linked, too.

[100487] In another embodiment thereof the expression cassette does not comprise an additional transit peptide in case in Table I, application no. 19, in column 6 in the same line as SEQ ID NO. 42502 is depicted, non-targeted is mentioned.

[100488] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 153813, or a homolog or fragment thereof, or a homolog thereof being depicted in Table I, application no. 19, column 8, in the same line as SEQ ID NO. 153813, preferably the coding region thereof, a homolog or a fragment thereof; which are operable linked.

[100489] In an embodiment thereof the expression cassette comprises in addition c) a nucleic acid molecule encoding a transit peptide, especially a plastidal transit petide in case in Table I, application no. 19, in column 6 in the same line as SEQ ID NO. 153813 is depicted, plastidic is mentioned, or a mitochondrial transit peptide in case in Table I, application no. 19, in column 6 in the same line as SEQ ID NO. 153813 is depicted, mitochondric is mentioned; which is operable linked, too.

[100490] In another embodiment thereof the expression cassette does not comprise an additional transit peptide in case in Table I, application no. 19, in column 6 in the same line as SEQ ID NO. 153813 is depicted, non-targeted is mentioned.

[100491] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 146472, or a homolog or fragment thereof, or a homolog thereof being depicted in Table I, application no. 19, column 8, in the same line as SEQ ID NO. 146472, preferably the coding region thereof, a homolog or a fragment thereof; which are operable linked.

[100492] In an embodiment thereof the expression cassette comprises in addition c) a nucleic acid molecule encoding a transit peptide, especially a plastidal transit petide in case in Table I, application no. 19, in column 6 in the same line as SEQ ID NO. 146472 is depicted, plastidic is mentioned, or a mitochondrial transit peptide in case in Table I, application no. 19, in column 6 in the same line as SEQ ID NO. 146472 is depicted, mitochondric is mentioned; which is operable linked, too.

[100493] In another embodiment thereof the expression cassette does not comprise an additional transit peptide in case in Table I, application no. 19, in column 6 in the same line as SEQ ID NO. 146472 is depicted, non-targeted is mentioned.

[100494] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 136098, or a homolog or fragment thereof, or a homolog thereof being depicted in Table I, application no. 19, column 8, in the same line as SEQ ID NO. 136098, preferably the coding region thereof, a homolog or a fragment thereof; which are operable linked.

[100495] In an embodiment thereof the expression cassette comprises in addition c) a nucleic acid molecule encoding a transit peptide, especially a plastidal transit petide in case in Table I, application no. 19, in column 6 in the same line as SEQ ID NO. 136098 is depicted, plastidic is mentioned, or a mitochondrial transit peptide in case in Table I, application no. 19, in column 6 in the same line as SEQ ID NO. 136098 is depicted, mitochondric is mentioned; which is operable linked, too.

[100496] In another embodiment thereof the expression cassette does not comprise an additional transit peptide in case in Table I, application no. 19, in column 6 in the same line as SEQ ID NO. 136098 is depicted, non-targeted is mentioned.

[100497] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 83726, or a homolog or fragment thereof, or a homolog thereof being depicted in Table I, application no. 19, column 8, in the same line as SEQ ID NO. 83726, preferably the coding region thereof, a homolog or a fragment thereof; which are operable linked.

[100498] In an embodiment thereof the expression cassette comprises in addition c) a nucleic acid molecule encoding a transit peptide, especially a plastidal transit petide in case in Table I, application no. 19, in column 6 in the same line as SEQ ID NO. 83726 is depicted, plastidic is mentioned, or a mitochondrial transit peptide in case in Table I, application no. 19, in column 6 in the same line as SEQ ID NO. 83726 is depicted, mitochondric is mentioned; which is operable linked, too.

[100499] In another embodiment thereof the expression cassette does not comprise an additional transit peptide in case in Table I, application no. 19, in column 6 in the same line as SEQ ID NO. 83726 is depicted, non-targeted is mentioned.

[100500] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 43800, or a homolog or fragment thereof, or a homolog thereof being depicted in Table I, application no. 19, column 8, in the same line as SEQ ID NO. 43800, preferably the coding region thereof, a homolog or a fragment thereof; which are operable linked.

[100501] In an embodiment thereof the expression cassette comprises in addition c) a nucleic acid molecule encoding a transit peptide, especially a plastidal transit petide in case in Table I, application no. 19, in column 6 in the same line as SEQ ID NO. 43800 is depicted, plastidic is mentioned, or a mitochondrial transit peptide in case in Table I, application no. 19, in column 6 in the same line as SEQ ID NO. 43800 is depicted, mitochondric is mentioned; which is operable linked, too.

[100502] In another embodiment thereof the expression cassette does not comprise an additional transit peptide in case in Table I, application no. 19, in column 6 in the same line as SEQ ID NO. 43800 is depicted, non-targeted is mentioned.

[100503] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 44372, or a homolog or fragment thereof, or a homolog thereof being depicted in Table I, application no. 19, column 8, in the same line as SEQ ID NO. 44372, preferably the coding region thereof, a homolog or a fragment thereof; which are operable linked.

[100504] In an embodiment thereof the expression cassette comprises in addition c) a nucleic acid molecule encoding a transit peptide, especially a plastidal transit petide in case in Table I, application no. 19, in column 6 in the same line as SEQ ID NO. 44372 is depicted, plastidic is mentioned, or a mitochondrial transit peptide in case in Table I, application no. 19, in column 6 in the same line as SEQ ID NO. 44372 is depicted, mitochondric is mentioned; which is operable linked, too.

[100505] In another embodiment thereof the expression cassette does not comprise an additional transit peptide in case in Table I, application no. 19, in column 6 in the same line as SEQ ID NO. 44372 is depicted, non-targeted is mentioned.

[100506] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 10740, or a homolog or fragment thereof, or a homolog thereof being depicted in Table I, application no. 19, column 8, in the same line as SEQ ID NO. 10740, preferably the coding region thereof, a homolog or a fragment thereof; which are operable linked.

[100507] In an embodiment thereof the expression cassette comprises in addition c) a nucleic acid molecule encoding a transit peptide, especially a plastidal transit petide in case in Table I, application no. 19, in column 6 in the same line as SEQ ID NO. 10740 is depicted, plastidic is mentioned, or a mitochondrial transit peptide in case in Table I, application no. 19, in column 6 in the same line as SEQ ID NO. 10740 is depicted, mitochondric is mentioned; which is operable linked, too.

[100508] In another embodiment thereof the expression cassette does not comprise an additional transit peptide in case in Table I, application no. 19, in column 6 in the same line as SEQ ID NO. 10740 is depicted, non-targeted is mentioned.

[100509] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 106532, or a homolog or fragment thereof, or a homolog thereof being depicted in Table I, application no. 19, column 8, in the same line as SEQ ID NO. 106532, preferably the coding region thereof, a homolog or a fragment thereof; which are operable linked.

[100510] In an embodiment thereof the expression cassette comprises in addition c) a nucleic acid molecule encoding a transit peptide, especially a plastidal transit petide in case in Table I, application no. 19, in column 6 in the same line as SEQ ID NO. 106532 is depicted, plastidic is mentioned, or a mitochondrial transit peptide in case in Table I, application no. 19, in column 6 in the same line as SEQ ID NO. 106532 is depicted, mitochondric is mentioned; which is operable linked, too.

[100511] In another embodiment thereof the expression cassette does not comprise an additional transit peptide in case in Table I, application no. 19, in column 6 in the same line as SEQ ID NO. 106532 is depicted, non-targeted is mentioned.

[100512] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 44662, or a homolog or fragment thereof, or a homolog thereof being depicted in Table I, application no. 19, column 8, in the same line as SEQ ID NO. 44662, preferably the coding region thereof, a homolog or a fragment thereof; which are operable linked.

[100513] In an embodiment thereof the expression cassette comprises in addition c) a nucleic acid molecule encoding a transit peptide, especially a plastidal transit petide in case in Table I, application no. 19, in column 6 in the same line as SEQ ID NO. 44662 is depicted, plastidic is mentioned, or a mitochondrial transit peptide in case in Table I, application no. 19, in column 6 in the same line as SEQ ID NO. 44662 is depicted, mitochondric is mentioned; which is operable linked, too.

[100514] In another embodiment thereof the expression cassette does not comprise an additional transit peptide in case in Table I, application no. 19, in column 6 in the same line as SEQ ID NO. 44662 is depicted, non-targeted is mentioned.

[100515] In an embodiment the present invention relates to an expresssion cassette comprising a) a promoter, preferably a promoter selected from the group consisting of Big35S, PCUbi, Super and USP, in particular p-USP; and b) the nucleic acid SEQ ID NO. 45556, or a homolog or fragment thereof, or a homolog thereof being depicted in Table I, application no. 19, column 8, in the same line as SEQ ID NO. 45556, preferably the coding region thereof, a homolog or a fragment thereof; which are operable linked.

[100516] In an embodiment thereof the expression cassette comprises in addition c) a nucleic acid molecule encoding a transit peptide, especially a plastidal transit petide in case in Table I, application no. 19, in column 6 in the same line as SEQ ID NO. 45556 is depicted, plastidic is mentioned, or a mitochondrial transit peptide in case in Table I, application no. 19, in column 6 in the same line as SEQ ID NO. 45556 is depicted, mitochondric is mentioned; which is operable linked, too.

[100517] In another embodiment thereof the expression cassette does not comprise an additional transit peptide in case in Table I, application no. 19, in column 6 in the same line as SEQ ID NO. 45556 is depicted, non-targeted is mentioned.

[100518] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 10811, or a homolog or fragment thereof, or a homolog thereof being depicted in Table I, application no. 19, column 8, in the same line as SEQ ID NO. 10811, preferably the coding region thereof, a homolog or a fragment thereof; which are operable linked.

[100519] In an embodiment thereof the expression cassette comprises in addition c) a nucleic acid molecule encoding a transit peptide, especially a plastidal transit petide in case in Table I, application no. 19, in column 6 in the same line as SEQ ID NO. 10811 is depicted, plastidic is mentioned, or a mitochondrial transit peptide in case in Table I, application no. 19, in column 6 in the same line as SEQ ID NO. 10811 is depicted, mitochondric is mentioned; which is operable linked, too.

[100520] In another embodiment thereof the expression cassette does not comprise an additional transit peptide in case in Table I, application no. 19, in column 6 in the same line as SEQ ID NO. 10811 is depicted, non-targeted is mentioned.

[100521] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 46405, or a homolog or fragment thereof, or a homolog thereof being depicted in Table I, application no. 19, column 8, in the same line as SEQ ID NO. 46405, preferably the coding region thereof, a homolog or a fragment thereof; which are operable linked.

[100522] In an embodiment thereof the expression cassette comprises in addition c) a nucleic acid molecule encoding a transit peptide, especially a plastidal transit petide in case in Table I, application no. 19, in column 6 in the same line as SEQ ID NO. 46405 is depicted, plastidic is mentioned, or a mitochondrial transit peptide in case in Table I, application no. 19, in column 6 in the same line as SEQ ID NO. 46405 is depicted, mitochondric is mentioned; which is operable linked, too.

[100523] In another embodiment thereof the expression cassette does not comprise an additional transit peptide in case in Table I, application no. 19, in column 6 in the same line as SEQ ID NO. 46405 is depicted, non-targeted is mentioned.

[100524] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 46751, or a homolog or fragment thereof, or a homolog thereof being depicted in Table I, application no. 19, column 8, in the same line as SEQ ID NO. 46751, preferably the coding region thereof, a homolog or a fragment thereof; which are operable linked.

[100525] In an embodiment thereof the expression cassette comprises in addition c) a nucleic acid molecule encoding a transit peptide, especially a plastidal transit petide in case in Table I, application no. 19, in column 6 in the same line as SEQ ID NO. 46751 is depicted, plastidic is mentioned, or a mitochondrial transit peptide in case in Table I, application no. 19, in column 6 in the same line as SEQ ID NO. 46751 is depicted, mitochondric is mentioned; which is operable linked, too.

[100526] In another embodiment thereof the expression cassette does not comprise an additional transit peptide in case in Table I, application no. 19, in column 6 in the same line as SEQ ID NO. 46751 is depicted, non-targeted is mentioned.

[100527] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 84198, or a homolog or fragment thereof, or a homolog thereof being depicted in Table I, application no. 19, column 8, in the same line as SEQ ID NO. 84198, preferably the coding region thereof, a homolog or a fragment thereof; which are operable linked.

[100528] In an embodiment thereof the expression cassette comprises in addition c) a nucleic acid molecule encoding a transit peptide, especially a plastidal transit petide in case in Table I, application no. 19, in column 6 in the same line as SEQ ID NO. 84198 is depicted, plastidic is mentioned, or a mitochondrial transit peptide in case in Table I, application no. 19, in column 6 in the same line as SEQ ID NO. 84198 is depicted, mitochondric is mentioned; which is operable linked, too.

[100529] In another embodiment thereof the expression cassette does not comprise an additional transit peptide in case in Table I, application no. 19, in column 6 in the same line as SEQ ID NO. 84198 is depicted, non-targeted is mentioned.

[100530] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 46850, or a homolog or fragment thereof, or a homolog thereof being depicted in Table I, application no. 19, column 8, in the same line as SEQ ID NO. 46850, preferably the coding region thereof, a homolog or a fragment thereof; which are operable linked.

[100531] In an embodiment thereof the expression cassette comprises in addition c) a nucleic acid molecule encoding a transit peptide, especially a plastidal transit petide in case in Table I, application no. 19, in column 6 in the same line as SEQ ID NO. 46850 is depicted, plastidic is mentioned, or a mitochondrial transit peptide in case in Table I, application no. 19, in column 6 in the same line as SEQ ID NO. 46850 is depicted, mitochondric is mentioned; which is operable linked, too.

[100532] In another embodiment thereof the expression cassette does not comprise an additional transit peptide in case in Table I, application no. 19, in column 6 in the same line as SEQ ID NO. 46850 is depicted, non-targeted is mentioned.

[100533] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 54337, or a homolog or fragment thereof, or a homolog thereof being depicted in Table I, application no. 19, column 8, in the same line as SEQ ID NO. 54337, preferably the coding region thereof, a homolog or a fragment thereof; which are operable linked.

[100534] In an embodiment thereof the expression cassette comprises in addition c) a nucleic acid molecule encoding a transit peptide, especially a plastidal transit petide in case in Table I, application no. 19, in column 6 in the same line as SEQ ID NO. 54337 is depicted, plastidic is mentioned, or a mitochondrial transit peptide in case in Table I, application no. 19, in column 6 in the same line as SEQ ID NO. 54337 is depicted, mitochondric is mentioned; which is operable linked, too.

[100535] In another embodiment thereof the expression cassette does not comprise an additional transit peptide in case in Table I, application no. 19, in column 6 in the same line as SEQ ID NO. 54337 is depicted, non-targeted is mentioned.

[100536] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 95047, or a homolog or fragment thereof, or a homolog thereof being depicted in Table I, application no. 19, column 8, in the same line as SEQ ID NO. 95047, preferably the coding region thereof, a homolog or a fragment thereof; which are operable linked.

[100537] In an embodiment thereof the expression cassette comprises in addition c) a nucleic acid molecule encoding a transit peptide, especially a plastidal transit petide in case in Table I, application no. 19, in column 6 in the same line as SEQ ID NO. 95047 is depicted, plastidic is mentioned, or a mitochondrial transit peptide in case in Table I, application no. 19, in column 6 in the same line as SEQ ID NO. 95047 is depicted, mitochondric is mentioned; which is operable linked, too.

[100538] In another embodiment thereof the expression cassette does not comprise an additional transit peptide in case in Table I, application no. 19, in column 6 in the same line as SEQ ID NO. 95047 is depicted, non-targeted is mentioned.

[100539] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 54452, or a homolog or fragment thereof, or a homolog thereof being depicted in Table I, application no. 19, column 8, in the same line as SEQ ID NO. 54452, preferably the coding region thereof, a homolog or a fragment thereof; which are operable linked.

[100540] In an embodiment thereof the expression cassette comprises in addition c) a nucleic acid molecule encoding a transit peptide, especially a plastidal transit petide in case in Table I, application no. 19, in column 6 in the same line as SEQ ID NO. 54452 is depicted, plastidic is mentioned, or a mitochondrial transit peptide in case in Table I, application no. 19, in column 6 in the same line as SEQ ID NO. 54452 is depicted, mitochondric is mentioned; which is operable linked, too.

[100541] In another embodiment thereof the expression cassette does not comprise an additional transit peptide in case in Table I, application no. 19, in column 6 in the same line as SEQ ID NO. 54452 is depicted, non-targeted is mentioned.

[100542] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 85808, or a homolog or fragment thereof, or a homolog thereof being depicted in Table I, application no. 19, column 8, in the same line as SEQ ID NO. 85808, preferably the coding region thereof, a homolog or a fragment thereof; which are operable linked.

[100543] In an embodiment thereof the expression cassette comprises in addition c) a nucleic acid molecule encoding a transit peptide, especially a plastidal transit petide in case in Table I, application no. 19, in column 6 in the same line as SEQ ID NO. 85808 is depicted, plastidic is mentioned, or a mitochondrial transit peptide in case in Table I, application no. 19, in column 6 in the same line as SEQ ID NO. 85808 is depicted, mitochondric is mentioned; which is operable linked, too.

[100544] In another embodiment thereof the expression cassette does not comprise an additional transit peptide in case in Table I, application no. 19, in column 6 in the same line as SEQ ID NO. 85808 is depicted, non-targeted is mentioned.

[100545] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 86447, or a homolog or fragment thereof, or a homolog thereof being depicted in Table I, application no. 19, column 8, in the same line as SEQ ID NO. 86447, preferably the coding region thereof, a homolog or a fragment thereof; which are operable linked.

[100546] In an embodiment thereof the expression cassette comprises in addition c) a nucleic acid molecule encoding a transit peptide, especially a plastidal transit petide in case in Table I, application no. 19, in column 6 in the same line as SEQ ID NO. 86447 is depicted, plastidic is mentioned, or a mitochondrial transit peptide in case in Table I, application no. 19, in column 6 in the same line as SEQ ID NO. 86447 is depicted, mitochondric is mentioned; which is operable linked, too.

[100547] In another embodiment thereof the expression cassette does not comprise an additional transit peptide in case in Table I, application no. 19, in column 6 in the same line as SEQ ID NO. 86447 is depicted, non-targeted is mentioned.

[100548] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 11471, or a homolog or fragment thereof, or a homolog thereof being depicted in Table I, application no. 19, column 8, in the same line as SEQ ID NO. 11471, preferably the coding region thereof, a homolog or a fragment thereof; which are operable linked.

[100549] In an embodiment thereof the expression cassette comprises in addition c) a nucleic acid molecule encoding a transit peptide, especially a plastidal transit petide in case in Table I, application no. 19, in column 6 in the same line as SEQ ID NO. 11471 is depicted, plastidic is mentioned, or a mitochondrial transit peptide in case in Table I, application no. 19, in column 6 in the same line as SEQ ID NO. 11471 is depicted, mitochondric is mentioned; which is operable linked, too.

[100550] In another embodiment thereof the expression cassette does not comprise an additional transit peptide in case in Table I, application no. 19, in column 6 in the same line as SEQ ID NO. 11471 is depicted, non-targeted is mentioned.

[100551] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 11990, or a homolog or fragment thereof, or a homolog thereof being depicted in Table I, application no. 19, column 8, in the same line as SEQ ID NO. 11990, preferably the coding region thereof, a homolog or a fragment thereof; which are operable linked.

[100552] In an embodiment thereof the expression cassette comprises in addition c) a nucleic acid molecule encoding a transit peptide, especially a plastidal transit petide in case in Table I, application no. 19, in column 6 in the same line as SEQ ID NO. 11990 is depicted, plastidic is mentioned, or a mitochondrial transit peptide in case in Table I, application no. 19, in column 6 in the same line as SEQ ID NO. 11990 is depicted, mitochondric is mentioned; which is operable linked, too.

[100553] In another embodiment thereof the expression cassette does not comprise an additional transit peptide in case in Table I, application no. 19, in column 6 in the same line as SEQ ID NO. 11990 is depicted, non-targeted is mentioned.

[100554] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 147407, or a homolog or fragment thereof, or a homolog thereof being depicted in Table I, application no. 19, column 8, in the same line as SEQ ID NO. 147407, preferably the coding region thereof, a homolog or a fragment thereof; which are operable linked.

[100555] In an embodiment thereof the expression cassette comprises in addition c) a nucleic acid molecule encoding a transit peptide, especially a plastidal transit petide in case in Table I, application no. 19, in column 6 in the same line as SEQ ID NO. 147407 is depicted, plastidic is mentioned, or a mitochondrial transit peptide in case in Table I, application no. 19, in column 6 in the same line as SEQ ID NO. 147407 is depicted, mitochondric is mentioned; which is operable linked, too.

[100556] In another embodiment thereof the expression cassette does not comprise an additional transit peptide in case in Table I, application no. 19, in column 6 in the same line as SEQ ID NO. 147407 is depicted, non-targeted is mentioned.

[100557] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 57734, or a homolog or fragment thereof, or a homolog thereof being depicted in Table I, application no. 19, column 8, in the same line as SEQ ID NO. 57734, preferably the coding region thereof, a homolog or a fragment thereof; which are operable linked.

[100558] In an embodiment thereof the expression cassette comprises in addition c) a nucleic acid molecule encoding a transit peptide, especially a plastidal transit petide in case in Table I, application no. 19, in column 6 in the same line as SEQ ID NO. 57734 is depicted, plastidic is mentioned, or a mitochondrial transit peptide in case in Table I, application no. 19, in column 6 in the same line as SEQ ID NO. 57734 is depicted, mitochondric is mentioned; which is operable linked, too.

[100559] In another embodiment thereof the expression cassette does not comprise an additional transit peptide in case in Table I, application no. 19, in column 6 in the same line as SEQ ID NO. 57734 is depicted, non-targeted is mentioned.

[100560] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 116341, or a homolog or fragment thereof, or a homolog thereof being depicted in Table I, application no. 19, column 8, in the same line as SEQ ID NO. 116341, preferably the coding region thereof, a homolog or a fragment thereof; which are operable linked.

[100561] In an embodiment thereof the expression cassette comprises in addition c) a nucleic acid molecule encoding a transit peptide, especially a plastidal transit petide in case in Table I, application no. 19, in column 6 in the same line as SEQ ID NO. 116341 is depicted, plastidic is mentioned, or a mitochondrial transit peptide in case in Table I, application no. 19, in column 6 in the same line as SEQ ID NO. 116341 is depicted, mitochondric is mentioned; which is operable linked, too.

[100562] In another embodiment thereof the expression cassette does not comprise an additional transit peptide in case in Table I, application no. 19, in column 6 in the same line as SEQ ID NO. 116341 is depicted, non-targeted is mentioned.

[100563] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 116460, or a homolog or fragment thereof, or a homolog thereof being depicted in Table I, application no. 19, column 8, in the same line as SEQ ID NO. 116460, preferably the coding region thereof, a homolog or a fragment thereof; which are operable linked.

[100564] In an embodiment thereof the expression cassette comprises in addition c) a nucleic acid molecule encoding a transit peptide, especially a plastidal transit petide in case in Table I, application no. 19, in column 6 in the same line as SEQ ID NO. 116460 is depicted, plastidic is mentioned, or a mitochondrial transit peptide in case in Table I, application no. 19, in column 6 in the same line as SEQ ID NO. 116460 is depicted, mitochondric is mentioned; which is operable linked, too.

[100565] In another embodiment thereof the expression cassette does not comprise an additional transit peptide in case in Table I, application no. 19, in column 6 in the same line as SEQ ID NO. 116460 is depicted, non-targeted is mentioned.

[100566] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 58324, or a homolog or fragment thereof, or a homolog thereof being depicted in Table I, application no. 19, column 8, in the same line as SEQ ID NO. 58324, preferably the coding region thereof, a homolog or a fragment thereof; which are operable linked.

[100567] In an embodiment thereof the expression cassette comprises in addition c) a nucleic acid molecule encoding a transit peptide, especially a plastidal transit petide in case in Table I, application no. 19, in column 6 in the same line as SEQ ID NO. 58324 is depicted, plastidic is mentioned, or a mitochondrial transit peptide in case in Table I, application no. 19, in column 6 in the same line as SEQ ID NO. 58324 is depicted, mitochondric is mentioned; which is operable linked, too.

[100568] In another embodiment thereof the expression cassette does not comprise an additional transit peptide in case in Table I, application no. 19, in column 6 in the same line as SEQ ID NO. 58324 is depicted, non-targeted is mentioned.

[100569] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 58590, or a homolog or fragment thereof, or a homolog thereof being depicted in Table I, application no. 19, column 8, in the same line as SEQ ID NO. 58590, preferably the coding region thereof, a homolog or a fragment thereof; which are operable linked.

[100570] In an embodiment thereof the expression cassette comprises in addition c) a nucleic acid molecule encoding a transit peptide, especially a plastidal transit petide in case in Table I, application no. 19, in column 6 in the same line as SEQ ID NO. 58590 is depicted, plastidic is mentioned, or a mitochondrial transit peptide in case in Table I, application no. 19, in column 6 in the same line as SEQ ID NO. 58590 is depicted, mitochondric is mentioned; which is operable linked, too.

[100571] In another embodiment thereof the expression cassette does not comprise an additional transit peptide in case in Table I, application no. 19, in column 6 in the same line as SEQ ID NO. 58590 is depicted, non-targeted is mentioned.

[100572] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 154137, or a homolog or fragment thereof, or a homolog thereof being depicted in Table I, application no. 19, column 8, in the same line as SEQ ID NO. 154137, preferably the coding region thereof, a homolog or a fragment thereof; which are operable linked.

[100573] In an embodiment thereof the expression cassette comprises in addition c) a nucleic acid molecule encoding a transit peptide, especially a plastidal transit petide in case in Table I, application no. 19, in column 6 in the same line as SEQ ID NO. 154137 is depicted, plastidic is mentioned, or a mitochondrial transit peptide in case in Table I, application no. 19, in column 6 in the same line as SEQ ID NO. 154137 is depicted, mitochondric is mentioned; which is operable linked, too.

[100574] In another embodiment thereof the expression cassette does not comprise an additional transit peptide in case in Table I, application no. 19, in column 6 in the same line as SEQ ID NO. 154137 is depicted, non-targeted is mentioned.

[100575] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 147457, or a homolog or fragment thereof, or a homolog thereof being depicted in Table I, application no. 19, column 8, in the same line as SEQ ID NO. 147457, preferably the coding region thereof, a homolog or a fragment thereof; which are operable linked.

[100576] In an embodiment thereof the expression cassette comprises in addition c) a nucleic acid molecule encoding a transit peptide, especially a plastidal transit petide in case in Table I, application no. 19, in column 6 in the same line as SEQ ID NO. 147457 is depicted, plastidic is mentioned, or a mitochondrial transit peptide in case in Table I, application no. 19, in column 6 in the same line as SEQ ID NO. 147457 is depicted, mitochondric is mentioned; which is operable linked, too.

[100577] In another embodiment thereof the expression cassette does not comprise an additional transit peptide in case in Table I, application no. 19, in column 6 in the same line as SEQ ID NO. 147457 is depicted, non-targeted is mentioned.

[100578] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 127521, or a homolog or fragment thereof, or a homolog thereof being depicted in Table I, application no. 19, column 8, in the same line as SEQ ID NO. 127521, preferably the coding region thereof, a homolog or a fragment thereof; which are operable linked.

[100579] In an embodiment thereof the expression cassette comprises in addition c) a nucleic acid molecule encoding a transit peptide, especially a plastidal transit petide in case in Table I, application no. 19, in column 6 in the same line as SEQ ID NO. 127521 is depicted, plastidic is mentioned, or a mitochondrial transit peptide in case in Table I, application no. 19, in column 6 in the same line as SEQ ID NO. 127521 is depicted, mitochondric is mentioned; which is operable linked, too.

[100580] In another embodiment thereof the expression cassette does not comprise an additional transit peptide in case in Table I, application no. 19, in column 6 in the same line as SEQ ID NO. 127521 is depicted, non-targeted is mentioned.

[100581] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 12341, or a homolog or fragment thereof, or a homolog thereof being depicted in Table I, application no. 19, column 8, in the same line as SEQ ID NO. 12341, preferably the coding region thereof, a homolog or a fragment thereof; which are operable linked.

[100582] In an embodiment thereof the expression cassette comprises in addition c) a nucleic acid molecule encoding a transit peptide, especially a plastidal transit petide in case in Table I, application no. 19, in column 6 in the same line as SEQ ID NO. 12341 is depicted, plastidic is mentioned, or a mitochondrial transit peptide in case in Table I, application no. 19, in column 6 in the same line as SEQ ID NO. 12341 is depicted, mitochondric is mentioned; which is operable linked, too.

[100583] In another embodiment thereof the expression cassette does not comprise an additional transit peptide in case in Table I, application no. 19, in column 6 in the same line as SEQ ID NO. 12341 is depicted, non-targeted is mentioned.

[100584] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 136907, or a homolog or fragment thereof, or a homolog thereof being depicted in Table I, application no. 19, column 8, in the same line as SEQ ID NO. 136907, preferably the coding region thereof, a homolog or a fragment thereof; which are operable linked.

[100585] In an embodiment thereof the expression cassette comprises in addition c) a nucleic acid molecule encoding a transit peptide, especially a plastidal transit petide in case in Table I, application no. 19, in column 6 in the same line as SEQ ID NO. 136907 is depicted, plastidic is mentioned, or a mitochondrial transit peptide in case in Table I, application no. 19, in column 6 in the same line as SEQ ID NO. 136907 is depicted, mitochondric is mentioned; which is operable linked, too.

[100586] In another embodiment thereof the expression cassette does not comprise an additional transit peptide in case in Table I, application no. 19, in column 6 in the same line as SEQ ID NO. 136907 is depicted, non-targeted is mentioned.

[100587] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 60301, or a homolog or fragment thereof, or a homolog thereof being depicted in Table I, application no. 19, column 8, in the same line as SEQ ID NO. 60301, preferably the coding region thereof, a homolog or a fragment thereof; which are operable linked.

[100588] In an embodiment thereof the expression cassette comprises in addition c) a nucleic acid molecule encoding a transit peptide, especially a plastidal transit petide in case in Table I, application no. 19, in column 6 in the same line as SEQ ID NO. 60301 is depicted, plastidic is mentioned, or a mitochondrial transit peptide in case in Table I, application no. 19, in column 6 in the same line as SEQ ID NO. 60301 is depicted, mitochondric is mentioned; which is operable linked, too.

[100589] In another embodiment thereof the expression cassette does not comprise an additional transit peptide in case in Table I, application no. 19, in column 6 in the same line as SEQ ID NO. 60301 is depicted, non-targeted is mentioned.

[100590] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 61553, or a homolog or fragment thereof, or a homolog thereof being depicted in Table I, application no. 19, column 8, in the same line as SEQ ID NO. 61553, preferably the coding region thereof, a homolog or a fragment thereof; which are operable linked.

[100591] In an embodiment thereof the expression cassette comprises in addition c) a nucleic acid molecule encoding a transit peptide, especially a plastidal transit petide in case in Table I, application no. 19, in column 6 in the same line as SEQ ID NO. 61553 is depicted, plastidic is mentioned, or a mitochondrial transit peptide in case in Table I, application no. 19, in column 6 in the same line as SEQ ID NO. 61553 is depicted, mitochondric is mentioned; which is operable linked, too.

[100592] In another embodiment thereof the expression cassette does not comprise an additional transit peptide in case in Table I, application no. 19, in column 6 in the same line as SEQ ID NO. 61553 is depicted, non-targeted is mentioned.

[100593] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 62079, or a homolog or fragment thereof, or a homolog thereof being depicted in Table I, application no. 19, column 8, in the same line as SEQ ID NO. 62079, preferably the coding region thereof, a homolog or a fragment thereof; which are operable linked.

[100594] In an embodiment thereof the expression cassette comprises in addition c) a nucleic acid molecule encoding a transit peptide, especially a plastidal transit petide in case in Table I, application no. 19, in column 6 in the same line as SEQ ID NO. 62079 is depicted, plastidic is mentioned, or a mitochondrial transit peptide in case in Table I, application no. 19, in column 6 in the same line as SEQ ID NO. 62079 is depicted, mitochondric is mentioned; which is operable linked, too.

[100595] In another embodiment thereof the expression cassette does not comprise an additional transit peptide in case in Table I, application no. 19, in column 6 in the same line as SEQ ID NO. 62079 is depicted, non-targeted is mentioned.

[100596] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 108092, or a homolog or fragment thereof, or a homolog thereof being depicted in Table I, application no. 19, column 8, in the same line as SEQ ID NO. 108092, preferably the coding region thereof, a homolog or a fragment thereof; which are operable linked.

[100597] In an embodiment thereof the expression cassette comprises in addition c) a nucleic acid molecule encoding a transit peptide, especially a plastidal transit petide in case in Table I, application no. 19, in column 6 in the same line as SEQ ID NO. 108092 is depicted, plastidic is mentioned, or a mitochondrial transit peptide in case in Table I, application no. 19, in column 6 in the same line as SEQ ID NO. 108092 is depicted, mitochondric is mentioned; which is operable linked, too.

[100598] In another embodiment thereof the expression cassette does not comprise an additional transit peptide in case in Table I, application no. 19, in column 6 in the same line as SEQ ID NO. 108092 is depicted, non-targeted is mentioned.

[100599] In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 62524, or a homolog or fragment thereof, or a homolog thereof being depicted in Table I, application no. 19, column 8, in the same line as SEQ ID NO. 62524, preferably the coding region thereof, a homolog or a fragment thereof; which are operable linked.

[100600] In an embodiment thereof the expression cassette comprises in addition c) a nucleic acid molecule encoding a transit peptide, especially a plastidal transit petide in case in Table I, application no. 19, in column 6 in the same line as SEQ ID NO. 62524 is depicted, plastidic is mentioned, or a mitochondrial transit peptide in case in Table I, application no. 19, in column 6 in the same line as SEQ ID NO. 62524 is depicted, mitochondric is mentioned; which is operable linked, too.

[100601] In another embodiment thereof the expression cassette does not comprise an additional transit peptide in case in Table I, application no. 19, in column 6 in the same line as SEQ ID NO. 62524 is depicted, non-targeted is mentioned.

[100602] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 119347, or a homolog or fragment thereof, or a homolog thereof being depicted in Table I, application no. 19, column 8, in the same line as SEQ ID NO. 119347, preferably the coding region thereof, a homolog or a fragment thereof; which are operable linked.

[100603] In an embodiment thereof the expression cassette comprises in addition c) a nucleic acid molecule encoding a transit peptide, especially a plastidal transit petide in case in Table I, application no. 19, in column 6 in the same line as SEQ ID NO. 119347 is depicted, plastidic is mentioned, or a mitochondrial transit peptide in case in Table I, application no. 19, in column 6 in the same line as SEQ ID NO. 119347 is depicted, mitochondric is mentioned; which is operable linked, too.

[100604] In another embodiment thereof the expression cassette does not comprise an additional transit peptide in case in Table I, application no. 19, in column 6 in the same line as SEQ ID NO. 119347 is depicted, non-targeted is mentioned.

[100605] In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 62717, or a homolog or fragment thereof, or a homolog thereof being depicted in Table I, application no. 19, column 8, in the same line as SEQ ID NO. 62717, preferably the coding region thereof, a homolog or a fragment thereof; which are operable linked.

[100606] In an embodiment thereof the expression cassette comprises in addition c) a nucleic acid molecule encoding a transit peptide, especially a plastidal transit petide in case in Table I, application no. 19, in column 6 in the same line as SEQ ID NO. 62717 is depicted, plastidic is mentioned, or a mitochondrial transit peptide in case in Table I, application no. 19, in column 6 in the same line as SEQ ID NO. 62717 is depicted, mitochondric is mentioned; which is operable linked, too.

[100607] In another embodiment thereof the expression cassette does not comprise an additional transit peptide in case in Table I, application no. 19, in column 6 in the same line as SEQ ID NO. 62717 is depicted, non-targeted is mentioned.

[100608] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 63264, or a homolog or fragment thereof, or a homolog thereof being depicted in Table I, application no. 19, column 8, in the same line as SEQ ID NO. 63264, preferably the coding region thereof, a homolog or a fragment thereof; which are operable linked.

[100609] In an embodiment thereof the expression cassette comprises in addition c) a nucleic acid molecule encoding a transit peptide, especially a plastidal transit petide in case in Table I, application no. 19, in column 6 in the same line as SEQ ID NO. 63264 is depicted, plastidic is mentioned, or a mitochondrial transit peptide in case in Table I, application no. 19, in column 6 in the same line as SEQ ID NO. 63264 is depicted, mitochondric is mentioned; which is operable linked, too.

[100610] In another embodiment thereof the expression cassette does not comprise an additional transit peptide in case in Table I, application no. 19, in column 6 in the same line as SEQ ID NO. 63264 is depicted, non-targeted is mentioned.

[100611] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 90159, or a homolog or fragment thereof, or a homolog thereof being depicted in Table I, application no. 19, column 8, in the same line as SEQ ID NO. 90159, preferably the coding region thereof, a homolog or a fragment thereof; which are operable linked.

[100612] In an embodiment thereof the expression cassette comprises in addition c) a nucleic acid molecule encoding a transit peptide, especially a plastidal transit petide in case in Table I, application no. 19, in column 6 in the same line as SEQ ID NO. 90159 is depicted, plastidic is mentioned, or a mitochondrial transit peptide in case in Table I, application no. 19, in column 6 in the same line as SEQ ID NO. 90159 is depicted, mitochondric is mentioned; which is operable linked, too.

[100613] In another embodiment thereof the expression cassette does not comprise an additional transit peptide in case in Table I, application no. 19, in column 6 in the same line as SEQ ID NO. 90159 is depicted, non-targeted is mentioned.

[100614] In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 63807, or a homolog or fragment thereof, or a homolog thereof being depicted in Table I, application no. 19, column 8, in the same line as SEQ ID NO. 63807, preferably the coding region thereof, a homolog or a fragment thereof; which are operable linked.

[100615] In an embodiment thereof the expression cassette comprises in addition c) a nucleic acid molecule encoding a transit peptide, especially a plastidal transit petide in case in Table I, application no. 19, in column 6 in the same line as SEQ ID NO. 63807 is depicted, plastidic is mentioned, or a mitochondrial transit peptide in case in Table I, application no. 19, in column 6 in the same line as SEQ ID NO. 63807 is depicted, mitochondric is mentioned; which is operable linked, too.

[100616] In another embodiment thereof the expression cassette does not comprise an additional transit peptide in case in Table I, application no. 19, in column 6 in the same line as SEQ ID NO. 63807 is depicted, non-targeted is mentioned.

[100617] In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 64144, or a homolog or fragment thereof, or a homolog thereof being depicted in Table I, application no. 19, column 8, in the same line as SEQ ID NO. 64144, preferably the coding region thereof, a homolog or a fragment thereof; which are operable linked.

[100618] In an embodiment thereof the expression cassette comprises in addition c) a nucleic acid molecule encoding a transit peptide, especially a plastidal transit petide in case in Table I, application no. 19, in column 6 in the same line as SEQ ID NO. 64144 is depicted, plastidic is mentioned, or a mitochondrial transit peptide in case in Table I, application no. 19, in column 6 in the same line as SEQ ID NO. 64144 is depicted, mitochondric is mentioned; which is operable linked, too.

[100619] In another embodiment thereof the expression cassette does not comprise an additional transit peptide in case in Table I, application no. 19, in column 6 in the same line as SEQ ID NO. 64144 is depicted, non-targeted is mentioned.

[100620] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 130742, or a homolog or fragment thereof, or a homolog thereof being depicted in Table I, application no. 19, column 8, in the same line as SEQ ID NO. 130742, preferably the coding region thereof, a homolog or a fragment thereof; which are operable linked.

[100621] In an embodiment thereof the expression cassette comprises in addition c) a nucleic acid molecule encoding a transit peptide, especially a plastidal transit petide in case in Table I, application no. 19, in column 6 in the same line as SEQ ID NO. 130742 is depicted, plastidic is mentioned, or a mitochondrial transit peptide in case in Table I, application no. 19, in column 6 in the same line as SEQ ID NO. 130742 is depicted, mitochondric is mentioned; which is operable linked, too.

[100622] In another embodiment thereof the expression cassette does not comprise an additional transit peptide in case in Table I, application no. 19, in column 6 in the same line as SEQ ID NO. 130742 is depicted, non-targeted is mentioned.

[100623] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 128655, or a homolog or fragment thereof, or a homolog thereof being depicted in Table I, application no. 19, column 8, in the same line as SEQ ID NO. 128655, preferably the coding region thereof, a homolog or a fragment thereof; which are operable linked.

[100624] In an embodiment thereof the expression cassette comprises in addition c) a nucleic acid molecule encoding a transit peptide, especially a plastidal transit petide in case in Table I, application no. 19, in column 6 in the same line as SEQ ID NO. 128655 is depicted, plastidic is mentioned, or a mitochondrial transit peptide in case in Table I, application no. 19, in column 6 in the same line as SEQ ID NO. 128655 is depicted, mitochondric is mentioned; which is operable linked, too.

[100625] In another embodiment thereof the expression cassette does not comprise an additional transit peptide in case in Table I, application no. 19, in column 6 in the same line as SEQ ID NO. 128655 is depicted, non-targeted is mentioned.

[100626] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 64336, or a homolog or fragment thereof, or a homolog thereof being depicted in Table I, application no. 19, column 8, in the same line as SEQ ID NO. 64336, preferably the coding region thereof, a homolog or a fragment thereof; which are operable linked.

[100627] In an embodiment thereof the expression cassette comprises in addition c) a nucleic acid molecule encoding a transit peptide, especially a plastidal transit petide in case in Table I, application no. 19, in column 6 in the same line as SEQ ID NO. 64336 is depicted, plastidic is mentioned, or a mitochondrial transit peptide in case in Table I, application no. 19, in column 6 in the same line as SEQ ID NO. 64336 is depicted, mitochondric is mentioned; which is operable linked, too.

[100628] In another embodiment thereof the expression cassette does not comprise an additional transit peptide in case in Table I, application no. 19, in column 6 in the same line as SEQ ID NO. 64336 is depicted, non-targeted is mentioned.

[100629] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 154394, or a homolog or fragment thereof, or a homolog thereof being depicted in Table I, application no. 19, column 8, in the same line as SEQ ID NO. 154394, preferably the coding region thereof, a homolog or a fragment thereof; which are operable linked.

[100630] In an embodiment thereof the expression cassette comprises in addition c) a nucleic acid molecule encoding a transit peptide, especially a plastidal transit petide in case in Table I, application no. 19, in column 6 in the same line as SEQ ID NO. 154394 is depicted, plastidic is mentioned, or a mitochondrial transit peptide in case in Table I, application no. 19, in column 6 in the same line as SEQ ID NO. 154394 is depicted, mitochondric is mentioned; which is operable linked, too.

[100631] In another embodiment thereof the expression cassette does not comprise an additional transit peptide in case in Table I, application no. 19, in column 6 in the same line as SEQ ID NO. 154394 is depicted, non-targeted is mentioned.

[100632] In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 14769, or a homolog or fragment thereof, or a homolog thereof being depicted in Table I, application no. 19, column 8, in the same line as SEQ ID NO. 14769, preferably the coding region thereof, a homolog or a fragment thereof; which are operable linked.

[100633] In an embodiment thereof the expression cassette comprises in addition c) a nucleic acid molecule encoding a transit peptide, especially a plastidal transit petide in case in Table I, application no. 19, in column 6 in the same line as SEQ ID NO. 14769 is depicted, plastidic is mentioned, or a mitochondrial transit peptide in case in Table I, application no. 19, in column 6 in the same line as SEQ ID NO. 14769 is depicted, mitochondric is mentioned; which is operable linked, too.

[100634] In another embodiment thereof the expression cassette does not comprise an additional transit peptide in case in Table I, application no. 19, in column 6 in the same line as SEQ ID NO. 14769 is depicted, non-targeted is mentioned.

[100635] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 149454, or a homolog or fragment thereof, or a homolog thereof being depicted in Table I, application no. 19, column 8, in the same line as SEQ ID NO. 149454, preferably the coding region thereof, a homolog or a fragment thereof; which are operable linked.

[100636] In an embodiment thereof the expression cassette comprises in addition c) a nucleic acid molecule encoding a transit peptide, especially a plastidal transit petide in case in Table I, application no. 19, in column 6 in the same line as SEQ ID NO. 149454 is depicted, plastidic is mentioned, or a mitochondrial transit peptide in case in Table I, application no. 19, in column 6 in the same line as SEQ ID NO. 149454 is depicted, mitochondric is mentioned; which is operable linked, too.

[100637] In another embodiment thereof the expression cassette does not comprise an additional transit peptide in case in Table I, application no. 19, in column 6 in the same line as SEQ ID NO. 149454 is depicted, non-targeted is mentioned.

[100638] In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 154437, or a homolog or fragment thereof, or a homolog thereof being depicted in Table I, application no. 19, column 8, in the same line as SEQ ID NO. 154437, preferably the coding region thereof, a homolog or a fragment thereof; which are operable linked.

[100639] In an embodiment thereof the expression cassette comprises in addition c) a nucleic acid molecule encoding a transit peptide, especially a plastidal transit petide in case in Table I, application no. 19, in column 6 in the same line as SEQ ID NO. 154437 is depicted, plastidic is mentioned, or a mitochondrial transit peptide in case in Table I, application no. 19, in column 6 in the same line as SEQ ID NO. 154437 is depicted, mitochondric is mentioned; which is operable linked, too.

[100640] In another embodiment thereof the expression cassette does not comprise an additional transit peptide in case in Table I, application no. 19, in column 6 in the same line as SEQ ID NO. 154437 is depicted, non-targeted is mentioned.

[100641] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 64964, or a homolog or fragment thereof, or a homolog thereof being depicted in Table I, application no. 19, column 8, in the same line as SEQ ID NO. 64964, preferably the coding region thereof, a homolog or a fragment thereof; which are operable linked.

[100642] In an embodiment thereof the expression cassette comprises in addition c) a nucleic acid molecule encoding a transit peptide, especially a plastidal transit petide in case in Table I, application no. 19, in column 6 in the same line as SEQ ID NO. 64964 is depicted, plastidic is mentioned, or a mitochondrial transit peptide in case in Table I, application no. 19, in column 6 in the same line as SEQ ID NO. 64964 is depicted, mitochondric is mentioned; which is operable linked, too.

[100643] In another embodiment thereof the expression cassette does not comprise an additional transit peptide in case in Table I, application no. 19, in column 6 in the same line as SEQ ID NO. 64964 is depicted, non-targeted is mentioned.

[100644] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 66274, or a homolog or fragment thereof, or a homolog thereof being depicted in Table I, application no. 19, column 8, in the same line as SEQ ID NO. 66274, preferably the coding region thereof, a homolog or a fragment thereof; which are operable linked.

[100645] In an embodiment thereof the expression cassette comprises in addition c) a nucleic acid molecule encoding a transit peptide, especially a plastidal transit petide in case in Table I, application no. 19, in column 6 in the same line as SEQ ID NO. 66274 is depicted, plastidic is mentioned, or a mitochondrial transit peptide in case in Table I, application no. 19, in column 6 in the same line as SEQ ID NO. 66274 is depicted, mitochondric is mentioned; which is operable linked, too.

[100646] In another embodiment thereof the expression cassette does not comprise an additional transit peptide in case in Table I, application no. 19, in column 6 in the same line as SEQ ID NO. 66274 is depicted, non-targeted is mentioned.

[100647] In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 150603, or a homolog or fragment thereof, or a homolog thereof being depicted in Table I, application no. 19, column 8, in the same line as SEQ ID NO. 150603, preferably the coding region thereof, a homolog or a fragment thereof; which are operable linked.

[100648] In an embodiment thereof the expression cassette comprises in addition c) a nucleic acid molecule encoding a transit peptide, especially a plastidal transit petide in case in Table I, application no. 19, in column 6 in the same line as SEQ ID NO. 150603 is depicted, plastidic is mentioned, or a mitochondrial transit peptide in case in Table I, application no. 19, in column 6 in the same line as SEQ ID NO. 150603 is depicted, mitochondric is mentioned; which is operable linked, too.

[100649] In another embodiment thereof the expression cassette does not comprise an additional transit peptide in case in Table I, application no. 19, in column 6 in the same line as SEQ ID NO. 150603 is depicted, non-targeted is mentioned.

[100650] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 154454, or a homolog or fragment thereof, or a homolog thereof being depicted in Table I, application no. 19, column 8, in the same line as SEQ ID NO. 154454, preferably the coding region thereof, a homolog or a fragment thereof; which are operable linked.

[100651] In an embodiment thereof the expression cassette comprises in addition c) a nucleic acid molecule encoding a transit peptide, especially a plastidal transit petide in case in Table I, application no. 19, in column 6 in the same line as SEQ ID NO. 154454 is depicted, plastidic is mentioned, or a mitochondrial transit peptide in case in Table I, application no. 19, in column 6 in the same line as SEQ ID NO. 154454 is depicted, mitochondric is mentioned; which is operable linked, too.

[100652] In another embodiment thereof the expression cassette does not comprise an additional transit peptide in case in Table I, application no. 19, in column 6 in the same line as SEQ ID NO. 154454 is depicted, non-targeted is mentioned.

[100653] In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 129065, or a homolog or fragment thereof, or a homolog thereof being depicted in Table I, application no. 19, column 8, in the same line as SEQ ID NO. 129065, preferably the coding region thereof, a homolog or a fragment thereof; which are operable linked.

[100654] In an embodiment thereof the expression cassette comprises in addition c) a nucleic acid molecule encoding a transit peptide, especially a plastidal transit petide in case in Table I, application no. 19, in column 6 in the same line as SEQ ID NO. 129065 is depicted, plastidic is mentioned, or a mitochondrial transit peptide in case in Table I, application no. 19, in column 6 in the same line as SEQ ID NO. 129065 is depicted, mitochondric is mentioned; which is operable linked, too.

[100655] In another embodiment thereof the expression cassette does not comprise an additional transit peptide in case in Table I, application no. 19, in column 6 in the same line as SEQ ID NO. 129065 is depicted, non-targeted is mentioned.

[100656] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 129123, or a homolog or fragment thereof, or a homolog thereof being depicted in Table I, application no. 19, column 8, in the same line as SEQ ID NO. 129123, preferably the coding region thereof, a homolog or a fragment thereof; which are operable linked.

[100657] In an embodiment thereof the expression cassette comprises in addition c) a nucleic acid molecule encoding a transit peptide, especially a plastidal transit petide in case in Table I, application no. 19, in column 6 in the same line as SEQ ID NO. 129123 is depicted, plastidic is mentioned, or a mitochondrial transit peptide in case in Table I, application no. 19, in column 6 in the same line as SEQ ID NO. 129123 is depicted, mitochondric is mentioned; which is operable linked, too.

[100658] In another embodiment thereof the expression cassette does not comprise an additional transit peptide in case in Table I, application no. 19, in column 6 in the same line as SEQ ID NO. 129123 is depicted, non-targeted is mentioned.

[100659] In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 139173, or a homolog or fragment thereof, or a homolog thereof being depicted in Table I, application no. 19, column 8, in the same line as SEQ ID NO. 139173, preferably the coding region thereof, a homolog or a fragment thereof; which are operable linked.

[100660] In an embodiment thereof the expression cassette comprises in addition c) a nucleic acid molecule encoding a transit peptide, especially a plastidal transit petide in case in Table I, application no. 19, in column 6 in the same line as SEQ ID NO. 139173 is depicted, plastidic is mentioned, or a mitochondrial transit peptide in case in Table I, application no. 19, in column 6 in the same line as SEQ ID NO. 139173 is depicted, mitochondric is mentioned; which is operable linked, too.

[100661] In another embodiment thereof the expression cassette does not comprise an additional transit peptide in case in Table I, application no. 19, in column 6 in the same line as SEQ ID NO. 139173 is depicted, non-targeted is mentioned.

[100662] In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 129470, or a homolog or fragment thereof, or a homolog thereof being depicted in Table I, application no. 19, column 8, in the same line as SEQ ID NO. 129470, preferably the coding region thereof, a homolog or a fragment thereof; which are operable linked.

[100663] In an embodiment thereof the expression cassette comprises in addition c) a nucleic acid molecule encoding a transit peptide, especially a plastidal transit petide in case in Table I, application no. 19, in column 6 in the same line as SEQ ID NO. 129470 is depicted, plastidic is mentioned, or a mitochondrial transit peptide in case in Table I, application no. 19, in column 6 in the same line as SEQ ID NO. 129470 is depicted, mitochondric is mentioned; which is operable linked, too.

[100664] In another embodiment thereof the expression cassette does not comprise an additional transit peptide in case in Table I, application no. 19, in column 6 in the same line as SEQ ID NO. 129470 is depicted, non-targeted is mentioned.

[100665] In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 129574, or a homolog or fragment thereof, or a homolog thereof being depicted in Table I, application no. 19, column 8, in the same line as SEQ ID NO. 129574, preferably the coding region thereof, a homolog or a fragment thereof; which are operable linked.

[100666] In an embodiment thereof the expression cassette comprises in addition c) a nucleic acid molecule encoding a transit peptide, especially a plastidal transit petide in case in Table I, application no. 19, in column 6 in the same line as SEQ ID NO. 129574 is depicted, plastidic is mentioned, or a mitochondrial transit peptide in case in Table I, application no. 19, in column 6 in the same line as SEQ ID NO. 129574 is depicted, mitochondric is mentioned; which is operable linked, too.

[100667] In another embodiment thereof the expression cassette does not comprise an additional transit peptide in case in Table I, application no. 19, in column 6 in the same line as SEQ ID NO. 129574 is depicted, non-targeted is mentioned.

[100668] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 154477, or a homolog or fragment thereof, or a homolog thereof being depicted in Table I, application no. 19, column 8, in the same line as SEQ ID NO. 154477, preferably the coding region thereof, a homolog or a fragment thereof; which are operable linked.

[100669] In an embodiment thereof the expression cassette comprises in addition c) a nucleic acid molecule encoding a transit peptide, especially a plastidal transit petide in case in Table I, application no. 19, in column 6 in the same line as SEQ ID NO. 154477 is depicted, plastidic is mentioned, or a mitochondrial transit peptide in case in Table I, application no. 19, in column 6 in the same line as SEQ ID NO. 154477 is depicted, mitochondric is mentioned; which is operable linked, too.

[100670] In another embodiment thereof the expression cassette does not comprise an additional transit peptide in case in Table I, application no. 19, in column 6 in the same line as SEQ ID NO. 154477 is depicted, non-targeted is mentioned.

[100671] In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 140110, or a homolog or fragment thereof, or a homolog thereof being depicted in Table I, application no. 19, column 8, in the same line as SEQ ID NO. 140110, preferably the coding region thereof, a homolog or a fragment thereof; which are operable linked.

[100672] In an embodiment thereof the expression cassette comprises in addition c) a nucleic acid molecule encoding a transit peptide, especially a plastidal transit petide in case in Table I, application no. 19, in column 6 in the same line as SEQ ID NO. 140110 is depicted, plastidic is mentioned, or a mitochondrial transit peptide in case in Table I, application no. 19, in column 6 in the same line as SEQ ID NO. 140110 is depicted, mitochondric is mentioned; which is operable linked, too.

[100673] In another embodiment thereof the expression cassette does not comprise an additional transit peptide in case in Table I, application no. 19, in column 6 in the same line as SEQ ID NO. 140110 is depicted, non-targeted is mentioned.

[100674] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 68413, or a homolog or fragment thereof, or a homolog thereof being depicted in Table I, application no. 19, column 8, in the same line as SEQ ID NO. 68413, preferably the coding region thereof, a homolog or a fragment thereof; which are operable linked.

[100675] In an embodiment thereof the expression cassette comprises in addition c) a nucleic acid molecule encoding a transit peptide, especially a plastidal transit petide in case in Table I, application no. 19, in column 6 in the same line as SEQ ID NO. 68413 is depicted, plastidic is mentioned, or a mitochondrial transit peptide in case in Table I, application no. 19, in column 6 in the same line as SEQ ID NO. 68413 is depicted, mitochondric is mentioned; which is operable linked, too.

[100676] In another embodiment thereof the expression cassette does not comprise an additional transit peptide in case in Table I, application no. 19, in column 6 in the same line as SEQ ID NO. 68413 is depicted, non-targeted is mentioned.

[100677] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 69, or a homolog or fragment thereof, or a homolog thereof being depicted in Table I, application no. 19, column 8, in the same line as SEQ ID NO. 69, preferably the coding region thereof, a homolog or a fragment thereof; which are operable linked.

[100678] In an embodiment thereof the expression cassette comprises in addition c) a nucleic acid molecule encoding a transit peptide, especially a plastidal transit petide in case in Table I, application no. 19, in column 6 in the same line as SEQ ID NO. 69 is depicted, plastidic is mentioned, or a mitochondrial transit peptide in case in Table I, application no. 19, in column 6 in the same line as SEQ ID NO. 69 is depicted, mitochondric is mentioned; which is operable linked, too.

[100679] In another embodiment thereof the expression cassette does not comprise an additional transit peptide in case in Table I, application no. 19, in column 6 in the same line as SEQ ID NO. 69 is depicted, non-targeted is mentioned.

[100680] [0165.1.7.19] to [0170.1.7.19] for the disclosure of these paragraphs see [0165.1.7.7] to [0170.1.7.7] above

[100681] The invention further provides a vector, comprising a nucleic acid molecule comprising 1) a nucleic acid molecule selected from the group consisting of:
[100682] (a) a nucleic acid molecule encoding a polypeptide shown in Table II, column 5 or 8, application no. 19, preferably shown in Table II A, application no. 19, in column 5, or in Table II

[100683] A, application no. 19, column 8, or in Table II B, application no. 19, column 8, or a homolog or a fragment thereof;
[100684] (b) a nucleic acid molecule shown in Table I, application no. 19, column 5 or 8, preferably shown in Table I A, application no. 19, in column 5, or in Table I A, application no. 19, column 8, or in Table I B, application no. 19, column 8, preferably the coding region thereof, or a homolog or a fragment thereof;
[100685] (c) a nucleic acid molecule, which, as a result of the degeneracy of the genetic code, can be derived from a polypeptide sequence depicted in column 5 or 8 of Table II, application no. 19, preferably shown in Table II A, application no. 19, in column 5, or in Table II A, application no. 19, column 8, or in Table II B, application no. 19, column 8;
[100686] (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 19, preferably shown in Table I A, application no. 19, in column 5, or in Table I A, application no. 19, column 8, or in Table I B, application no. 19, column 8, preferably the coding region thereof;
[100687] (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
[100688] (f) a nucleic acid molecule which hybridizes with a nucleic acid molecule of (a), (b), (c), (d), or (e) under stringent hybridization conditions;
[100689] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
[100690] (h) a nucleic acid molecule encoding a polypeptide comprising the consensus sequence or one or more polypeptide motifs as shown in column 8 of Table IV, application no. 19;
[100691] (i) a nucleic acid molecule encoding a polypeptide having the activity represented by a protein as depicted in column 5 of Table II, application no. 19;
[100692] (j) a nucleic acid molecule comprising a polynucleotide which is obtained by amplifying a cDNA library or a genomic library using the primers in Table III, application no. 19, column 8;
[100693] and
[100694] (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
[100695] or a nucleic acid molecule comprising a sequence which is complementary thereto.
   (All the references to Tables I to IV are references to the corresponding lines of Tables I to IV.)

[100696] In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 19.

[100697] In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wildtype non-human organism or part thereof.

[100698] In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 19, and confers a generation or an increase of the respective fine chemical in a non-human organism or a part thereof as compared to a corresponding wild-type non-human organism or part thereof.

[100699] In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

[100700] In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 19.

[100701] In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[100702] In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 19, and confers a generation or an increase of the respective fine chemical in a non-human organism or a part thereof as compared to a corresponding wild-type non-human organism or part thereof.

[100703] In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 19, in the same line as of the respective nucleic acid molecule according to 1) “plastidic” is depicted, or encoding a mitochondrial transit peptide, especially in case in column 6 of Table I, application no. 19, in the same line as of the respective nucleic acid molecule according to 1) “mitochondric” is depicted, which are operable linked.

[100704] In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 19, in the same line as of the respective nucleic acid molecule according to 1) “non-targeted” is depicted.

[100705] In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 19, in the same line as of the respective nucleic acid molecule according to 1) “plastidic” is depicted, or encoding a mitochondrial transit peptide, especially in case in column 6 of Table I, application no. 19, in the same line as of the respective nucleic acid molecule according to 1) “mitochondric” is depicted, which are operable linked.

[100706] In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operable linked; a nucleic acid molecule encoding a transit peptide, especially in case in column 6 of Table I, application no. 19, in the same line as of the respective nucleic acid molecule according to 1) “non-targeted” is depicted.

[100707] In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 19, in the same line as of the respective nucleic acid molecule according to 1) “plastidic” is depicted, or encoding a mitochondrial transit peptide, especially in case in column 6 of Table I, application no. 19, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 19 (in the same line).

[100708] In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 19, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 19.

[100709] In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 19, in the same line as of the respective nucleic acid molecule according to 1) “plastidic” is depicted, or encoding a mitochondrial transit peptide, especially in case in column 6 of Table I, application no. 19, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[100710] In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 19, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[100711] In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 19, in the same line as of the respective nucleic acid molecule according to 1) “plastidic” is depicted, or encoding a mitochondrial transit peptide, especially in case in column 6 of Table I, application no. 19, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 19 and confers a generation or an increase of the respective fine chemical in a non-human organism or a part thereof as compared to a corresponding wild-type non-human organism or part thereof.

[100712] In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 19, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 19 (in the same line) and confers a generation or an increase of the respective fine chemical in a non-human organism or a part thereof as compared to a corresponding wild-type non-human organism or part thereof.

[100713] In a preferred embodiment of said vector said nucleic acid molecule according to 1 (a), (b), (c), (d), (e), (f), (g), (h), (i), (j) or (k), said promoter and optionally said nucleic acid molecule encoding a transit peptide are encompassed in an expression cassette. Accordingly in an embodiment the invention provides a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

[100714] The invention further provides a vector comprising a nucleic acid molecule comprising 1) a nucleic acid molecule selected from the group consisting of:
[100715] (a) a nucleic acid molecule encoding a polypeptide shown in Table II, column 5 or 8, application no. 19, preferably shown in Table II A, application no. 19, in column 5, or in Table II A, application no. 19, column 8, or in Table II B, application no. 19, column 8, or a homolog or a fragment thereof;
[100716] (b) a nucleic acid molecule shown in Table I, application no. 19, column 5 or 8, preferably shown in Table I A, application no. 19, in column 5, or in Table I A, application no. 19, column 8, or in Table I B, application no. 19, column 8, preferably the coding region thereof, or a homolog or a fragment thereof;
[100717] (c) a nucleic acid molecule, which, as a result of the degeneracy of the genetic code, can be derived from a polypeptide sequence depicted in column 5 or 8 of Table II, application no. 19, preferably shown in Table II A, application no. 19, in column 5, or in Table II A, application no. 19, column 8, or in Table II B, application no. 19, column 8;
[100718] (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 19, preferably shown in Table I A, application no. 19, in column 5, or in Table I A, application no. 19, column 8, or in Table I B, application no. 19, column 8, preferably the coding region thereof;
[100719] (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
[100720] (f) a nucleic acid molecule which hybridizes with a nucleic acid molecule of (a), (b), (c), (d), or (e) under stringent hybridization conditions;
[100721] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
[100722] (h) a nucleic acid molecule encoding a polypeptide comprising the consensus sequence or one or more polypeptide motifs as shown in column 8 of Table IV, application no. 19;
[100723] (i) a nucleic acid molecule encoding a polypeptide having the activity represented by a protein as depicted in column 5 of Table II, application no. 19;
[100724] (j) a nucleic acid molecule comprising a polynucleotide which is obtained by amplifying a cDNA library or a genomic library using the primers in Table III, application no. 19, column 8;
[100725] and
[100726] (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
[100727] or a nucleic acid molecule comprising a sequence which is complementary thereto;
   whereby, preferably, the nucleic acid molecule according to 1 (a), (b), (c), (d), (e), (f), (g), (h), (i), (j) or (k) distinguishes over the sequence depicted in Table I A and/or I B, application no. 19, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 19, column 5 or 8, or the coding region thereof, respectively. In another embodiment, the nucleic acid molecule of the present invention, preferably the coding region thereof, is 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 19, column 5 or 8, or the coding region thereof, respectively. In a further embodiment the nucleic acid molecule does not encode the polypeptide sequence shown in Table II A and/or II B, application no. 19, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 19, column 5 or 8 and does not encode a protein of the sequence shown in Table II A and/or II B, application no. 19, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 19, column 5 and 8, or the coding region thereof. In a further embodiment, the protein of the present invention is at least 30%,40%, 50%, 60%, 70%, 80%, 90%, 95% identical to protein sequence depicted in Table II A and/or II B, application no. 19, column 5 or 8 but less than 100%, preferably less than 99.999%, 99.99% or 99.9%, more preferably less than 99%, 985, 97%, 96% or 95% identical to the sequence shown in Table II A and/or II B, application no. 19, column 5 or 8.
   (All the references to Tables I to IV are references to the corresponding lines of Tables I to IV.)

[100728] In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 19.

[100729] In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wildtype non-human organism or part thereof.

[100730] In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 19, and confers a generation or an increase of the respective fine chemical in a non-human organism or a part thereof as compared to a corresponding wild-type non-human organism or part thereof.

[100731] In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

[100732] In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 19.

[100733] In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[100734] In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 19, and confers a generation or an increase of the respective fine chemical in a non-human organism or a part thereof as compared to a corresponding wild-type non-human organism or part thereof.

[100735] In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 19, in the same line as of the respective nucleic acid molecule according to 1) “plastidic” is depicted, or encoding a mitochondrial transit peptide, especially in case in column 6 of Table I, application no. 19, in the same line as of the respective nucleic acid molecule according to 1) “mitochondric” is depicted, which are operable linked.

[100736] In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 19, in the same line as of the respective nucleic acid molecule according to 1) “non-targeted” is depicted.

[100737] In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 19, in the same line as of the respective nucleic acid molecule according to 1) “plastidic” is depicted, or encoding a mitochondrial transit peptide, especially in case in column 6 of Table I, application no. 19, in the same line as of the respective nucleic acid molecule according to 1) “mitochondric” is depicted, which are operable linked.

[100738] In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 19, in the same line as of the respective nucleic acid molecule according to 1) “non-targeted” is depicted.

[100739] In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 19, in the same line as of the respective nucleic acid molecule according to 1) “plastidic” is depicted, or encoding a mitochondrial transit peptide, especially in case in column 6 of Table I, application no. 19, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 19.

[100740] In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 19, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 19.

[100741] In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 19, in the same line as of the respective nucleic acid molecule according to 1) “plastidic” is depicted, or encoding a mitochondrial transit peptide, especially in case in column 6 of Table I, application no. 19, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[100742] In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 19, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[100743] In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 19, in the same line as of the respective nucleic acid molecule according to 1) “plastidic” is depicted, or encoding a mitochondrial transit peptide, especially in case in column 6 of Table I, application no. 19, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 19 and confers a generation or an increase of the respective fine chemical in a non-human organism or a part thereof as compared to a corresponding wild-type non-human organism or part thereof.

[100744] In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 19, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 19 and confers a generation or an increase of the respective fine chemical in a non-human organism or a part thereof as compared to a corresponding wild-type non-human organism or part thereof.

[100745] [0181.1.7.19] to [0209.1.7.19] for the disclosure of these paragraphs see [0181.1.7.7] to [0209.1.7.7] above

[100746] The genes of the invention, coding in one embodiment for an activity selected from the group consisting of (DL)-glycerol-3-phosphatase, 2-C-methyl-D-erythritol 4-phosphate cytidylyltransferase, 3-oxoacyl-[acyl-carrier-protein] synthase, 49747384_SOYBEAN-protein, ABC transporter permease protein, acetyl CoA carboxylase, acid phosphatase precursor, acid shock protein, acyl transferase, adenylate kinase, alcohol dehydrogenase, amino-acid acetyltransferase, arginine decarboxylase, argininosuccinate synthetase, aromatic acid decarboxylase, At1g23110-protein, At1g67340-protein, ATP-binding component of a transport system, ATP-dependent RNA helicase, b0050-protein, b0518-protein, b0970-protein, b1003-protein, b1234-protein, b1330-protein, b1522-protein, b1556-protein, b1583-protein, b1670-protein, b1672-protein, b1703-protein, B1795-protein, b1837-protein, b2032-protein, b2099-protein, b2139-protein, b2399-protein, b2474-protein, b2613-protein, b2673-protein, b3083-protein, b3160-protein, b3777-protein, b3791-protein, b3989-protein, b4029-protein, b4050-protein, beta-hydroxylase, biotin synthase, calcium-dependent protein kinase, carbamoyl-phosphate synthase subunit, carbohydrate kinase, cardiolipin synthase, CBL-interacting protein kinase, CDP-diacylglycerol-glycerol-3-phosphate 3-phosphatidyltransferase, cell division control protein, cell division protein, coproporphyrinogen III oxidase, cyclin, cyclin D , dihydrolipoamide dehydrogenase, DnaJ-like chaperone, Elongation factor Tu, enterobacterial common antigen polymerase, ethanolamine utilization protein, eukaryotic translation initiation factor 5, Fe—S subunit of oxidoreductase, FKBP-type peptidyl-prolyl cis-trans isomerase (rotamase), glutamate 5-kinase, glutaredoxin, Glycine cleavage system T aminomethyltransferase, GM02LC17485-protein, heat shock protein Hsp40, heat shock transcription factor, HesB/YadR/YfhF family protein, high-affinity branched-chain amino acid transport protein, hydrolase, imidazole glycerol phosphate synthase subunit, ion-transport protein, lipoprotein precursor, L-serine dehydratase, lysozyme, lysyl-tRNA synthetase, metal-dependent hydrolase, methylglyoxal synthase, methyltransferase, Mg-protoporphyrin IX monomethyl ester oxidative cyclase 66 kD subunit, mitochondrial processing protease, mitogen-activated protein kinase, molecular chaperone portein, monothiol glutaredoxin, murein transglycosylase, NADH dehydrogenase, NADH-quinone oxidoreductase subunit, nitrate/nitrite transport protein, oxidoreductase, phenylacetic acid degradation protein, phosphate import ATP-binding protein, phosphatidylinositol 3- and 4-kinase family protein, phosphoglyceromutase, phosphotransferase, phosphotransferase system component, photosystem I reaction centre subunit, polygalacturonase, precorrin-8X methylmutase, protein phosphatase, purine-nucleoside phosphorylase, putative transport system permease protein, putrescine transport system permease protein, pyruvate kinase, recombinase A, secindependent protein translocase, serine protease, sodium/proton antiporter, ThiF family protein, threonine dehydratase, threonine synthase, threonine/serine transporter, transcription factor, transcriptional regulator, transcriptional regulator protein, trehalose-phosphatase, TTC1386-protein, vacuolar sorting protein, YAL013W-protein, ycr102c-protein, ydr273w-protein, YFL019C-protein, YHR202W-protein, YIL083C-protein, YJL062W-A-protein, YLR099C-protein, ylr326w-protein, YPL162C.protein, and Zm_{—}4842_BE510522-protein are also called “FCRP genes”.

[100747] [0211.1.7.19] to [0225.1.7.19] for the disclosure of these paragraphs see [0211.1.7.7] to [0225.1.7.7] above

[100748] In addition to the sequence mentioned in Table I, application no. 19, column 5 or 8, preferably the coding region thereof, or its homologs or fragments, it may be advantageous additionally to express and/or mutate further genes in the non-human organisms. Especially advantageously, additionally at least one further gene of the biosynthetic pathway of the fine chemical is expressed in the non-human organisms such as plants or microorganisms. It is also possible that the regulation of the natural genes has been modified advantageously so that the gene and/or its gene product is no longer subject to the regulatory mechanisms which exist in the non-human organisms. This leads to an increased synthesis of the respective fine chemical glycerol and/or derivates since, for example, feedback regulations no longer exist to the same extent or not at all. In addition it might be advantageously to combine the nucleic acids sequences of the invention containing the sequences shown in the respective line in Table I, application no. 19, column 5 or 8, preferably the coding region thereof, or homologs or fragments thereof, with genes which generally support or enhance the growth or yield of the target non-human organisms, for example genes which lead to faster growth rate of non human organism like microorganisms or plants or genes which produce stress-, pathogen-, or herbicide-resistant plants.

[100749] In a further embodiment of the process of the invention, non-human organisms are grown, in which there is in addition to the expression of the nucleic acid molecule according to the invention simultaneous expression of at least one nucleic acid molecule or one of the genes which code for proteins involved in the of the glycerol synthesis pathway such as glycerol biosynthesis chain such as hexokinase, glucose-3-P-dehydrogenase, phosphofructokinase, aldolase, glycerol-3-P-dehydrogenase etc.

[100750] [0228.1.7.19] to [0239.1.7.19] for the disclosure of these paragraphs see [0228.1.7.7] to [0239.1.7.7] above

[100751] In an embodiment of the present invention the nucleic acid construct of the present invention comprises a nucleic acid molecule as depicted in the respective line in Table I, application no. 19, column 5 or 8, preferably the coding region thereof, a fragment or a homolog thereof, and a plant promoter, such as the Big 35S promoter, the DC3 promoter, the PC Ubi promoter, the LegB4-promoter, the Super promoter, the USP promoter or the like. In another embodiment the nucleic acid construct of the present invention comprises a nucleic acid molecule as depicted in the respective line in Table I, application no. 19, column 5 or 8, preferably the coding region thereof, a fragment or a homolog thereof, and a plant promoter such as, for example, the USP, the LegB4-, the DC3 promoter or the ubiquitin promoter from parsley or other herein mentioned promoters. In a further embodiment the nucleic acid construct of the present invention comprises a nucleic acid molecule as depicted in the respective line in Table I, application no. 19, column 5 or 8, preferably the coding region thereof, a fragment or a homolog thereof, and a plant promoter, such as the Big 35S promoter, the PC Ubi promoter, the Super promoter, the USP promoter or the like. In addition different terminators may advantageously be used in these nucleic acid constructs; examples for transcriptional termination are polyadenylation signals.

[100752] [0241.1.7.19] to [0245.1.7.19] for the disclosure of these paragraphs see [0241.1.7.7] to [0245.1.7.7] above

[100753] Other preferred sequences for use in operable linkage in gene expression constructs are targeting sequences, which are required for targeting the gene product into specific cell compartments (for a review, see Kermode, Crit. Rev. Plant Sci. 15 (4), 285 (1996) and references cited therein), for example into the vacuole, the nucleus, all types of plastids, such as amyloplasts, chloroplasts, chromoplasts, the extracellular space, the mitochondria, the endoplasmic reticulum, elaioplasts, peroxisomes, glycosomes, and other compartments of cells or extracellular; preferred are sequences, which are involved in targeting to plastids as mentioned above. Sequences, which must be mentioned in this context are, in particular, the signalpeptideor transit-peptide-encoding sequences which are known per se. For example, plastid-transit-peptide-encoding sequences enable the targeting of the expression product into the plastids of a plant cell. Targeting sequences are also known for eukaryotic and to a lower extent for prokaryotic organisms and can advantageously be operable linked with the nucleic acid molecule of the present invention as shown in Table I, application no. 19, columns 5 and 8, preferably the coding region thereof, or homologs or fragments thereof, and described herein to achieve an expression in one of said compartments or extracellular.

[100754] [0247.1.7.19] to [0249.1.7.19] for the disclosure of these paragraphs see [0247.1.7.7] to [0249.1.7.7] above

[100755] Preferred promoters are in particular those which bring about gene expression in tissues and organs, such as leaves, roots, seeds, pollen or xylem. Examples of tissue preferred or organ preferred promoters include, but are not limited to fruit-preferred, ovule-preferred, male tissue-preferred, seed-preferred, pollen-preferred integument-preferred, tuber-preferred, stalk-preferred, pericarp-preferred, leaf-preferred, stigma-preferred, pollen-preferred, anther-preferred, petal-preferred, sepal-preferred, pedicel-preferred, silique-preferred, stem-preferred, root-preferred promoters, and the like. Especially those promoters are preferred which bring about gene expression in tissues and organs in which the biosynthesis of glycerol and/or derivates and/or lipids takes place, like in seed cells, such as endosperm cells and cells of the developing embryo. Seed promoters are preferentially expressed during seed development and/or germination. For example, seed preferred promoters can be embryo-preferred, endosperm preferred and seed coat-preferred (see Thompson et al., BioEssays 10, 108 (1989)). Examples of seed preferred promoters include, but are not limited to, cellulose synthase (celA), Cim1, gamma-zein, globulin-1, maize 19 kD zein (cZ19B1), and the like. Other suitable promoters are the oilseed rape napin gene promoter (U.S. Pat. No. 5,608,152), the Vicia faba USP promoter (Baeumlein et al., Mol Gen Genet, 225 (3), 459 (1991)), the Arabidopsis oleosin promoter (WO 98/45461), the Phaseolus vulgaris phaseolin promoter (U.S. Pat. No. 5,504,200), the Brassica Bce4 promoter (WO 91/13980), the bean arcs promoter, the carrot DcG3 promoter, or the Legumin B4 promoter (LeB4) (Baeumlein et al., Plant Journal, 2 (2), 233 (1992)), and promoters which bring about the seed-specific expression in monocotyledonous plants such as maize, barley, wheat, rye, rice and the like. Advantageous seed-specific promoters are the sucrose binding protein promoter (WO 00/26388), the phaseolin promoter and the napin promoter. Suitable promoters which must be considered are the barley Ipt2 or Ipt1 gene promoter (WO 95/15389 and WO 95/23230), and the promoters described in WO 99/16890 (promoters from the barley hordein gene, the rice glutelin gene, the rice oryzin gene, the rice prolamin gene, the wheat gliadin gene, the wheat glutelin gene, the maize zein gene, the oat glutelin gene, the sorghum kasirin gene and the rye secalin gene). Further suitable promoters are Amy32b, Amy 6-6 and Aleurain (U.S. Pat. No. 5,677,474), Bce4 (oilseed rape) (U.S. Pat. No. 5,530,149), glycinin (soya) (EP 571 741], phosphoenolpyruvate carboxylase (soya) (JP 06/62870), ADR12-2 (soya) (WO 98/08962), isocitrate lyase (oilseed rape) (U.S. Pat. No. 5,689,040]) or α-amylase (barley) (EP 781 8499. Other promoters which are available for the expression of genes in plants are leaf-specific promoters such as those described in DE-A 19 644 478 or light-regulated promoters such as, for example, the pea petE promoter.

[100756] [0251.1.7.19] to [0266.1.7.19] for the disclosure of these paragraphs see [0251.1.7.7] to [0266.1.7.7] above

[100757] An embodiment of the invention relates to a vector where the nucleic acid molecule according to the invention is linked operably to regulatory sequences which permit the expression in a prokaryotic or eukaryotic host. A further embodiment of the invention relates to a vector in which a nucleic acid sequence encoding one of the polypeptides shown in the respective line in Table II, application no. 19, columns 5 or 8, or homologs or fragments thereof, is functionally linked to a targeting sequence such as a plastidial or mitochondrial targeting sequence. A further embodiment of the invention relates to a vector in which a nucleic acid sequence encoding one of the polypeptides shown in the respective line in Table II, application no. 19, columns 5 or 8, or homologs or fragments thereof, is functionally linked to (a) regulatory sequence which permit the expression in an organelle, such as plastids or mitochondria, preferably plastids.

[100758] A further embodiment of the invention relates to a vector which comprises a nucleic acid sequence encoding one of the polypeptides shown in the respective line in Table II, application no. 19, columns 5 or 8, or homologs or fragments thereof, but no additional targeting sequence.

[100759] [0268.1.7.19] to [0273.1.7.19] for the disclosure of these paragraphs see [0268.1.7.7] to [0273.1.7.7] above

[100760] A further object of the invention relates to the use of a nucleic acid construct, e.g. an expression cassette, comprising one or more nucleic acid molecules encoding one or more polypeptides as depicted in the respective line(s) in Table II, application no. 19, columns 5 or 8, or homologs or fragments thereof, or comprising one or more nucleic acid molecules as depicted in the respective line(s) in Table I, application no. 19, columns 5 or 8, preferably the coding region thereof, or homologs or fragments thereof, or comprising a nucleic acid molecule hybridizing therewith for the transformation of a microorganism or a plant cell, plant tissues or a part of a plant

[100761] In doing so, depending on the choice of promoter, the nucleic acid molecules shown in the respective line in Table I, application no. 19, column 5 or 8, preferably the coding region thereof, or homologs or fragments thereof, or the nucleic acid molecules encoding a polypeptide as depicted in the respective line in Table II, application no. 19, column 5 or 8, or homologs or fragments thereof, can be expressed in the whole plant or specifically e.g. in the leaves, in the seeds, the nodules, in roots, in the stem or other parts of the plant. Those transgenic plants overproducing sequences, e.g. as depicted in the respective line in Table I, application no. 19, column 5 or 8, preferably the coding region thereof, or homologs or fragments thereof, the plant cell, plant tissues, plants, the reproductive material thereof, or parts thereof are a further object of the present invention.

[100762] Within the framework of the present invention, the production of the bis-glyceryl phosphate (polar fraction), glycerol (lipid fraction), glycerol (polar fraction), glycerol phosphate (lipid fraction), glycerol-3-phosphate (polar fraction), or triacylglycerol is due to the generation or over-expression of one or more polypeptides as depicted in the respective line(s) in Table II, application no. 19, column 5 or 8, or homologs or fragments thereof, or encoded by the corresponding nucleic acid molecules as depicted in the respective line(s) in Table I, application no. 19, column 5 or 8, preferably the coding region thereof, or homologs or fragments thereof, in the non-human organism according to the invention, advantageously in the transgenic microorganism or plant according to the invention, at least for the duration of at least one plant generation.

[100763] A constitutive expression of the polypeptide as depicted in the respective line in Table II, application no. 19, column 5 or 8, or homologs or fragments thereof, or encoded by the respective nucleic acid molecule as depicted in the respective line in Table I, application no. 19, column 5 or 8, preferably the coding region thereof, or homologs or fragments thereof, may be advantageous. On the other hand, an inducible expression may also appear desirable. Expression of the polypeptide sequences of the invention can be either directed to the cytosol or to the organelles, such as plastids or mitochondria, preferably the plastids of the host cells, preferably the plant cells, or non-targeted.

[100764] for the disclosure of this paragraph see [0278.1.7.7] above

[100765] In a further embodiment the invention relates to a transgenic non-human organisms such as transgenic microorganism or transgenic plant comprising a nucleic acid molecule comprising
[100766] [1) a nucleic acid molecule selected from the group consisting of
   [100767] (a) a nucleic acid molecule encoding a polypeptide shown in Table II, application no. 19, column 5 or 8, preferably shown in Table II A, application no. 19, column 5, or in Table II A, application no. 19, column 8, or in Table II B, application no. 19, column 8, or a homolog or a fragment thereof;
   [100768] (b) a nucleic acid molecule shown in Table I, application no. 19, column 5 or 8, preferably shown in Table I A, application no. 19, column 5, or in Table I A, application no. 19, column 8, or in Table I B, application no. 19, column 8, preferably the coding region thereof, or a homolog or a fragment thereof;
   [100769] (c) a nucleic acid molecule, which, as a result of the degeneracy of the genetic code, can be derived from a polypeptide sequence depicted in column 5 or 8 of Table II, application no. 19, preferably shown in Table II A, application no. 19, column 5, or in Table II A, application no. 19, column 8, or in Table II B, application no. 19, column 8;
   [100770] (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 19, preferably shown in Table I A, application no. 19, column 5, or in Table I A, application no. 19, column 8, or in Table I B, application no. 19, column 8, preferably the coding region thereof;
   [100771] (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   [100772] (f) a nucleic acid molecule which hybridizes with a nucleic acid molecule of (a), (b), (c), (d) or (e) under stringent hybridization conditions;
   [100773] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   [100774] (h) a nucleic acid molecule encoding a polypeptide comprising the consensus sequence or one or more polypeptide motifs as shown in column 8 of Table IV, application no. 19;
   [100775] (i) a nucleic acid molecule encoding a polypeptide having the activity represented by a respective protein as depicted in column 5 of Table II, application no. 19;
   [100776] (j) a nucleic acid molecule comprising a polynucleotide which is obtained by amplifying a cDNA library or a genomic library using the primers in Table III, application no. 19, column 8;
   [100777] and
   [100778] (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   [100779] or a nucleic acid molecule comprising a sequence which is complementary thereto; and, if desired
[100780] 2) nucleic acid molecule encoding a transit peptide, preferably encoding a plastidal transit peptide or a mitochondrial transit peptide;
   which are operable linked;]
   or comprising an expression cassette comprising
[100781] [0) a promoter, preferably a promoter selected from the group consisting of Big35S, PCUbi, Super and USP; and
[100782] 1) a nucleic acid molecule selected from the group consisting of
   [100783] (a) a nucleic acid molecule encoding a polypeptide shown in Table II, application no. 19, column 5 or 8, preferably shown in Table II A, application no. 19, column 5, or in Table II A, application no. 19, column 8, or in Table II B, application no. 19, column 8, or a homolog or a fragment thereof;
   [100784] (b) a nucleic acid molecule shown in Table I, application no. 19, column 5 or 8, preferably shown in Table I A, application no. 19, column 5, or in Table I A, application no. 19, column 8, or in Table I B, application no. 19, column 8, preferably the coding region thereof, or a homolog or a fragment thereof;
   [100785] (c) a nucleic acid molecule, which, as a result of the degeneracy of the genetic code, can be derived from a polypeptide sequence depicted in column 5 or 8 of Table II, application no. 19, preferably shown in Table II A, application no. 19, column 5, or in Table II A, application no. 19, column 8, or in Table II B, application no. 19, column 8;
   [100786] (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 19, preferably shown in Table I A, application no. 19, column 5, or in Table I A, application no. 19, column 8, or in Table I B, application no. 19, column 8, preferably the coding region thereof;
   [100787] (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   [100788] (f) a nucleic acid molecule which hybridizes with a nucleic acid molecule of (a), (b), (c), (d) or (e) under stringent hybridization conditions;
   [100789] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   [100790] (h) a nucleic acid molecule encoding a polypeptide comprising the consensus sequence or one or more polypeptide motifs as shown in column 8 of Table IV, application no. 19;
   [100791] (i) a nucleic acid molecule encoding a polypeptide having the activity represented by a respective protein as depicted in column 5 of Table II, application no. 19;
   [100792] (j) a nucleic acid molecule comprising a polynucleotide which is obtained by amplifying a cDNA library or a genomic library using the primers in Table III, application no. 19, column 8;
   [100793] and
   [100794] (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   [100795] or a nucleic acid molecule comprising a sequence which is complementary thereto;
      and, if desired
[100796] 2) nucleic acid molecule encoding a transit peptide, preferably encoding a plastidal transit peptide or a mitochondrial transit peptide;
   which are operable linked;]
   or comprising or being transformed by a vector comprising
[100797] [1) a nucleic acid molecule selected from the group consisting of
   [100798] (a) a nucleic acid molecule encoding a polypeptide shown in Table II, application no. 19, column 5 or 8, preferably shown in Table II A, application no. 19, column 5, or in Table II A, application no. 19, column 8, or in Table II B, application no. 19, column 8, or a homolog or a fragment thereof;
   [100799] (b) a nucleic acid molecule shown in Table I, application no. 19, column 5 or 8, preferably shown in Table I A, application no. 19, column 5, or in Table I A, application no. 19, column 8, or in Table I B, application no. 19, column 8, preferably the coding region thereof, or a homolog or a fragment thereof;
   [100800] (c) a nucleic acid molecule, which, as a result of the degeneracy of the genetic code, can be derived from a polypeptide sequence depicted in column 5 or 8 of Table II, application no. 19, preferably shown in Table II A, application no. 19, column 5, or in Table II A, application no. 19, column 8, or in Table II B, application no. 19, column 8;
   [100801] (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 19, preferably shown in Table I A, application no. 19, column 5, or in Table I A, application no. 19, column 8, or in Table I B, application no. 19, column 8, preferably the coding region thereof;
   [100802] (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   [100803] (f) a nucleic acid molecule which hybridizes with a nucleic acid molecule of (a), (b), (c), (d) or (e) under stringent hybridization conditions;
   [100804] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   [100805] (h) a nucleic acid molecule encoding a polypeptide comprising the consensus sequence or one or more polypeptide motifs as shown in column 8 of Table IV, application no. 19;
   [100806] (i) a nucleic acid molecule encoding a polypeptide having the activity represented by a respective protein as depicted in column 5 of Table II, application no. 19;
   [100807] (j) a nucleic acid molecule comprising a polynucleotide which is obtained by amplifying a cDNA library or a genomic library using the primers in Table III, application no. 19, column 8;
   [100808] and
   [100809] (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   [100810] or a nucleic acid molecule comprising a sequence which is complementary thereto;]
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

[100811] In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 19.

[100812] In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[100813] In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 19 and confers a generation or an increase of the respective fine chemical in a non-human organism or a part thereof as compared to a corresponding wild-type non-human organism or part thereof.

[100814] In another embodiment of said transgenic non-human organism the expression cassette comprises 2) a nucleic acid molecule encoding a transit peptide, preferably encoding a plastidal transit peptide, especially in case in column 6 of Table I, application no. 19, in the same line as of the respective nucleic acid molecule according to 1) “plastidic” is depicted, or encoding a mitochondrial transit peptide, especially in case in column 6 of Table I, application no. 19, in the same line as of the respective nucleic acid molecule according to 1) “mitochondric” is depicted.

[100815] In another embodiment of said transgenic non-human organism the expression cassette does not comprise 2) a nucleic acid molecule encoding a transit peptide, especially in case in column 6 of Table I, application no. 19, in the same line as of the respective nucleic acid molecule according to 1) “non-targeted” is depicted.

[100816] In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

[100817] In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 19.

[100818] In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[100819] In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 19, and confers a generation or an increase of the respective fine chemical in a non-human organism or a part thereof as compared to a corresponding wild-type non-human organism or part thereof.

[100820] In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 19, in the same line as of the respective nucleic acid molecule according to 1) “plastidic” is depicted, or encoding a mitochondrial transit peptide, especially in case in column 6 of Table I, application no. 19, in the same line as of the respective nucleic acid molecule according to 1) “mitochondric” is depicted, which are operable linked.

[100821] In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 19, in the same line as of the respective nucleic acid molecule according to 1) “non-targeted” is depicted, which are operable linked.

[100822] In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 19, in the same line as of the respective nucleic acid molecule according to 1) “plastidic” is depicted, or encoding a mitochondrial transit peptide, especially in case in column 6 of Table I, application no. 19, in the same line as of the respective nucleic acid molecule according to 1) “mitochondric” is depicted, which are operable linked.

[100823] In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 19, in the same line as of the respective nucleic acid molecule according to 1) “non-targeted” is depicted, which are operable linked.

[100824] In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 19, in the same line as of the respective nucleic acid molecule according to 1) “plastidic” is depicted, or encoding a mitochondrial transit peptide, especially in case in column 6 of Table I, application no. 19, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 19.

[100825] In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 19, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 19.

[100826] In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 19, in the same line as of the respective nucleic acid molecule according to 1) “plastidic” is depicted, or encoding a mitochondrial transit peptide, especially in case in column 6 of Table I, application no. 19, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[100827] In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 19, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[100828] In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 19, in the same line as of the respective nucleic acid molecule according to 1) “plastidic” is depicted, or encoding a mitochondrial transit peptide, especially in case in column 6 of Table I, application no. 19, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 19 and confers a generation or an increase of the respective fine chemical in a non-human organism or a part thereof as compared to a corresponding wild-type non-human organism or part thereof.

[100829] In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 19, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 19 (in the same line) and confers a generation or an increase of the respective fine chemical in a non-human organism or a part thereof as compared to a corresponding wild-type non-human organism or part thereof.

[100830] In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

[100831] In a further embodiment the invention relates to a transgenic non-human organisms such as transgenic microorganism or transgenic plant comprising a nucleic acid molecule comprising
[100832] [1) a nucleic acid molecule selected from the group consisting of
   [100833] (a) a nucleic acid molecule encoding a polypeptide shown in Table II, application no. 19, column 5 or 8, preferably shown in Table II A, application no. 19, column 5, or in Table II A, application no. 19, column 8, or in Table II B, application no. 19, column 8, or a homolog or a fragment thereof;
   [100834] (b) a nucleic acid molecule shown in Table I, application no. 19, column 5 or 8, preferably shown in Table I A, application no. 19, column 5, or in Table I A, application no. 19, column 8, or in Table I B, application no. 19, column 8, preferably the coding region thereof, or a homolog or a fragment thereof;
   [100835] (c) a nucleic acid molecule, which, as a result of the degeneracy of the genetic code, can be derived from a polypeptide sequence depicted in column 5 or 8 of Table II, application no. 19, preferably shown in Table II A, application no. 19, column 5, or in Table II A, application no. 19, column 8, or in Table II B, application no. 19, column 8;
   [100836] (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 19, preferably shown in Table I A, application no. 19, column 5, or in Table I A, application no. 19, column 8, or in Table I B, application no. 19, column 8, preferably the coding region thereof;
   [100837] (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   [100838] (f) a nucleic acid molecule which hybridizes with a nucleic acid molecule of (a), (b), (c), (d) or (e) under stringent hybridization conditions;
   [100839] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   [100840] (h) a nucleic acid molecule encoding a polypeptide comprising the consensus sequence or one or more polypeptide motifs as shown in column 8 of Table IV, application no. 19;
   [100841] (i) a nucleic acid molecule encoding a polypeptide having the activity represented by a respective protein as depicted in column 5 of Table II, application no. 19;
   [100842] (j) a nucleic acid molecule comprising a polynucleotide which is obtained by amplifying a cDNA library or a genomic library using the primers in Table III, application no. 19, column 8;
   [100843] and
   [100844] (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   [100845] or a nucleic acid molecule comprising a sequence which is complementary thereto;
      and, if desired
[100846] 2) nucleic acid molecule encoding a transit peptide, preferably encoding a plastidal transit peptide or a mitochondrial transit peptide;
   which are operable linked;]
   or comprising an expression cassette comprising
[100847] [0) a promoter, preferably a promoter selected from the group consisting of Big35S, PCUbi, Super and USP; and
[100848] 1) a nucleic acid molecule selected from the group consisting of
   [100849] (a) a nucleic acid molecule encoding a polypeptide shown in Table II, application no. 19, column 5 or 8, preferably shown in Table II A, application no. 19, column 5, or in Table II A, application no. 19, column 8, or in Table II B, application no. 19, column 8, or a homolog or a fragment thereof;
   [100850] (b) a nucleic acid molecule shown in Table I, application no. 19, column 5 or 8, preferably shown in Table I A, application no. 19, column 5, or in Table I A, application no. 19, column 8, or in Table I B, application no. 19, column 8, preferably the coding region thereof, or a homolog or a fragment thereof;
   [100851] (c) a nucleic acid molecule, which, as a result of the degeneracy of the genetic code, can be derived from a polypeptide sequence depicted in column 5 or 8 of Table II, application no. 19, preferably shown in Table II A, application no. 19, column 5, or in Table II A, application no. 19, column 8, or in Table II B, application no. 19, column 8;
   [100852] (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 19, preferably shown in Table I A, application no. 19, column 5, or in Table I A, application no. 19, column 8, or in Table I B, application no. 19, column 8, preferably the coding region thereof;
   [100853] (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   [100854] (f) a nucleic acid molecule which hybridizes with a nucleic acid molecule of (a), (b), (c), (d) or (e) under stringent hybridization conditions;
   [100855] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   [100856] (h) a nucleic acid molecule encoding a polypeptide comprising the consensus sequence or one or more polypeptide motifs as shown in column 8 of Table IV, application no. 19;
   [100857] (i) a nucleic acid molecule encoding a polypeptide having the activity represented by a respective protein as depicted in column 5 of Table II, application no. 19;
   [100858] (j) a nucleic acid molecule comprising a polynucleotide which is obtained by amplifying a cDNA library or a genomic library using the primers in Table III, application no. 19, column 8;
   [100859] and
   [100860] (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   [100861] or a nucleic acid molecule comprising a sequence which is complementary thereto;]
      and, if desired
[100862] 2) nucleic acid molecule encoding a transit peptide, preferably encoding a plastidal transit peptide or a mitochondrial transit peptide;
   which are operable linked;]
   or comprising or being transformed by a vector comprising
[100863] [1) a nucleic acid molecule selected from the group consisting of
   [100864] (a) a nucleic acid molecule encoding a polypeptide shown in Table II, application no. 19, column 5 or 8, preferably shown in Table II A, application no. 19, column 5, or in Table II A, application no. 19, column 8, or in Table II B, application no. 19, column 8, or a homolog or a fragment thereof;
   [100865] (b) a nucleic acid molecule shown in Table I, application no. 19, column 5 or 8, preferably shown in Table I A, application no. 19, column 5, or in Table I A, application no. 19, column 8, or in Table I B, application no. 19, column 8, preferably the coding region thereof, or a homolog or a fragment thereof;
   [100866] (c) a nucleic acid molecule, which, as a result of the degeneracy of the genetic code, can be derived from a polypeptide sequence depicted in column 5 or 8 of Table II, application no. 19, preferably shown in Table II A, application no. 19, column 5, or in Table II A, application no. 19, column 8, or in Table II B, application no. 19, column 8;
   [100867] (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 19, preferably shown in Table I A, application no. 19, column 5, or in Table I A, application no. 19, column 8, or in Table I B, application no. 19, column 8, preferably the coding region thereof;
   [100868] (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   [100869] (f) a nucleic acid molecule which hybridizes with a nucleic acid molecule of (a), (b), (c), (d) or (e) under stringent hybridization conditions;
   [100870] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   [100871] (h) a nucleic acid molecule encoding a polypeptide comprising the consensus sequence or one or more polypeptide motifs as shown in column 8 of Table IV, application no. 19;
   [100872] (i) a nucleic acid molecule encoding a polypeptide having the activity represented by a respective protein as depicted in column 5 of Table II, application no. 19;
   [100873] (k) a nucleic acid molecule comprising a polynucleotide which is obtained by amplifying a cDNA library or a genomic library using the primers in Table III, application no. 19, column 8;
   [100874] and
   [100875] (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   [100876] or a nucleic acid molecule comprising a sequence which is complementary thereto;]
      whereby, preferably, the nucleic acid molecule according to 1 (a), (b), (c), (d), (e), (f), (g), (h), (i), (j) or (k) distinguishes over the sequence depicted in Table I A and/or I B, application no. 19, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 19, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 19, column 5 or 8, or the coding region thereof, respectively. In a further embodiment the nucleic acid molecule does not encode the polypeptide sequence shown in Table II A and/or II B, application no. 19, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 19, column 5 or 8 and does not encode a protein of the sequence shown in Table II A and/or II B, application no. 19, column 5 or 8. Accordingly, in one embodiment, the protein encoded by a sequence of a nucleic acid according to 1 (a), (b), (c), (d), (e), (f), (g), (h), (i), (j) or (k) does not consist of the sequence shown in Table I A and/or I B, application no. 19, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 19, column 5 or 8 but less than 100%, preferably less than 99.999%, 99.99%, 99.9%, 99%, 98%, 97%, 96% or 95% identical to the sequence shown in Table II A and/or II B, application no. 19, column 5 or 8;
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

[100877] In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 19.

[100878] In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[100879] In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 19 and confers a generation or an increase of the respective fine chemical in a non-human organism or a part thereof as compared to a corresponding wild-type non-human organism or part thereof.

[100880] In another embodiment of said transgenic non-human organism the expression cassette comprises 2) a nucleic acid molecule encoding a transit peptide, preferably encoding a plastidal transit peptide, especially in case in column 6 of Table I, application no. 19, in the same line as of the respective nucleic acid molecule according to 1) “plastidic” is depicted, or encoding a mitochondrial transit peptide, especially in case in column 6 of Table I, application no. 19, in the same line as of the respective nucleic acid molecule according to 1) “mitochondric” is depicted.

[100881] In another embodiment thereof the expression cassette does not comprise 2) a nucleic acid molecule encoding a transit peptide, especially in case in column 6 of Table I, application no. 19, in the same line as of the respective nucleic acid molecule according to 1) “non-targeted” is depicted.

[100882] In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

[100883] In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 19.

[100884] In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[100885] In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the same line in column 5 of Table II, application no. 19, and confers a generation or an increase of the respective fine chemical in a non-human organism or a part thereof as compared to a corresponding wild-type non-human organism or part thereof.

[100886] In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 19, in the same line as of the respective nucleic acid molecule according to 1) “plastidic” is depicted, or encoding a mitochondrial transit peptide, especially in case in column 6 of Table I, application no. 19, in the same line as of the respective nucleic acid molecule according to 1) “mitochondric” is depicted, which are operable linked.

[100887] In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 19, in the same line as of the respective nucleic acid molecule according to 1) “non-targeted” is depicted, which are operable linked.

[100888] In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 19, in the same line as of the respective nucleic acid molecule according to 1) “plastidic” is depicted, or encoding a mitochondrial transit peptide, especially in case in column 6 of Table I, application no. 19, in the same line as of the respective nucleic acid molecule according to 1) “mitochondric” is depicted, which are operable linked.

[100889] In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 19, in the same line as of the respective nucleic acid molecule according to 1) “non-targeted” is depicted, which are operable linked.

[100890] In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 19, in the same line as of the respective nucleic acid molecule according to 1) “plastidic” is depicted, or encoding a mitochondrial transit peptide, especially in case in column 6 of Table I, application no. 19, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 19.

[100891] In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 19, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 19.

[100892] In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 19, in the same line as of the respective nucleic acid molecule according to 1) “plastidic” is depicted, or encoding a mitochondrial transit peptide, especially in case in column 6 of Table I, application no. 19, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[100893] In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 19, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[100894] In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 19, in the same line as of the respective nucleic acid molecule according to 1) “plastidic” is depicted, or encoding a mitochondrial transit peptide, especially in case in column 6 of Table I, application no. 19, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 19 and confers a generation or an increase of the respective fine chemical in a non-human organism or a part thereof as compared to a corresponding wild-type non-human organism or part thereof.

[100895] In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 19, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 19 and confers a generation or an increase of the respective fine chemical in a non-human organism or a part thereof as compared to a corresponding wild-type non-human organism or part thereof.

[100896] In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

[100897] [0291.1.7.19] to [0299.1.7.19] for the disclosure of these paragraphs see [0291.1.7.7] to [0299.1.7.7] above

[100898] The term “biological active portion” or “biological activity” means a polypeptide as depicted in the respective line in Table II, application no. 19, column 3 or a portion of said polypeptide which still has at least 10% or 20%, preferably 30%, 40%, 50% or 60%, especially preferably 70%, 75%, 80%, 90% or 95% of the enzymatic or biological activity of the natural or starting enzyme or protein.

[100899] [0301.1.7.19] to [0304.1.7.19] for the disclosure of these paragraphs see [0301.1.7.7] to [0304.1.7.7] above

[100900] Synthetic oligonucleotide primers for the amplification, e.g. as shown in the respective line in Table III, application no. 19, column 8, by means of polymerase chain reaction can be generated on the basis of a sequence shown herein, for example the sequence shown in the respective line in Table I, application no. 19, columns 5 or 8, or the sequences derived from Table II, application no. 19, columns 5 or 8, respectively.

[100901] Moreover, it is possible to identify a conserved region of a protein from various organism by carrying out protein sequence alignments with the polypeptide encoded by the nucleic acid molecules of the present invention, in particular with the sequences encoded by the nucleic acid molecule shown in the respective line in column 5 or 8 of Table I, application no. 19, from which conserved regions, and in turn, degenerate primers can be derived. Conserved regions are those, which show a very little variation in the amino acid in one particular position of several homologs from different origin. The consensus sequence and polypeptide motifs shown in the respective line in column 8 of Table IV, application no. 19, are derived from such alignments of homologous proteins.

[100902] In an embodiment of the present invention, in the method of the present invention the activity of a polypeptide comprising or consisting of a consensus sequence or at least one polypeptide motif shown in the respective line in Table IV, application no. 19, column 8, is increased. In another embodiment thereof, the method of the present invention relates to a polypeptide comprising or consisting of a consensus sequence shown in the respective line in Table IV, application no. 19, column 8 or comprising at least one polypeptide motif shown in the respective line in Table IV, application no. 19, column 8, whereby not more than 20, 15, 10, 9, 8, 7, 6, 5 4, 3, 2 or 1, or 0 of the amino acid positions indicated can be replaced by any amino acid. In another embodiment thereof, the method of the present invention relates to a polypeptide comprising or consisting of a consensus sequence shown in the respective line in Table IV, application no. 19, column 8 or comprising at least one polypeptide motif shown in the respective line in Table IV, application no. 19, column 8, whereby not more than 15%, 10%, 5%, 4%, 3%, 2% or 1%, or 0% of the amino acid positions defined as distinct specific amino acids are/is replaced by another amino acid. In another embodiment thereof, the method of the present invention relates to a polypeptide comprising or consisting of a consensus sequence shown in the respective line in Table IV, application no. 19, column 8 or comprising at least one polypeptide motif shown in the respective line in Table IV, application no. 19, column 8, whereby not more than 20, 15, 10, 9, 8, 7, 6, 5, 4, 3, 2 or 1, or 0 amino acids are inserted into a consensus sequence or polypeptide motif.

[100903] In an embodiment of the present invention, in the method of the present invention the activity of a polypeptide comprising or consisting of a consensus sequence or at least one polypeptide motif shown in the respective line in Table IV, application no. 19, column 8, is increased, whereby said polypeptide distinguishes over the sequence depicted in Table II, application no. 19, columns 5 or 8 by one or more amino acids.

[100904] In one embodiment, said polypeptide of the invention distinguishes over the sequence shown in the respective line in Table II, application no. 19, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II B, application no. 19, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in Table II, application no. 19, columns 5 or 8.

[100905] [0309.1.7.19] to [0321.1.7.19] for the disclosure of these paragraphs see [0309.1.7.7] to [0321.1.7.7] above

[100906] Polypeptides having above-mentioned activity, i.e. conferring the production or the increased production of the fine chemical bis-glyceryl phosphate (polar fraction), glycerol (lipid fraction), glycerol (polar fraction), glycerol phosphate (lipid fraction), glycerol-3-phosphate (polar fraction), or triacylglycerol as compared to a corresponding, e.g. non-transformed, wild type non-human organism, like a microorganism or a plant cell, plant or part thereof, can be encoded by other DNA sequences which hybridize to the sequences shown in the respective line in Table I, application no. 19, columns 5 and 8, preferably the coding region thereof, at least under relaxed hybridization conditions and which encode the expression of polypeptides conferring the production or the increased production of the respective fine chemical bis-glyceryl phosphate (polar fraction), glycerol (lipid fraction), glycerol (polar fraction), glycerol phosphate (lipid fraction), glycerol-3-phosphate (polar fraction), or triacylglycerol as compared to a corresponding, e.g. non-transformed, wild type non-human organism, like a microorganism or a plant cell, plant or part thereof.

[100907] [0323.1.7.19] to [0329.1.7.19] for the disclosure of these paragraphs see [0323.1.7.7] to [0329.1.7.7] above

[100908] Further, the nucleic acid molecule of the invention comprises a nucleic acid molecule, which is a complement of one of the nucleotide sequences of above-mentioned nucleic acid molecules or a portion thereof. A nucleic acid molecule or its sequence which is complementary to one of the nucleotide molecules or sequences shown in the respective line in Table I, application no. 19, columns 5 or 8, preferably the coding region thereof, homologs or fragments thereof, is one which is sufficiently complementary to one of the nucleotide molecules or sequences shown in the respective line in Table I, application no. 19, columns 5 or 8, preferably the coding region thereof, homologs or fragments thereof, such that it can hybridize to one of the nucleotide sequences shown in Table I, application no. 19, columns 5 or 8, preferably the coding region thereof, homologs or fragments thereof, thereby forming a stable duplex. Preferably, the hybridization is performed under stringent hybridization conditions. However, a complement of one of the herein disclosed sequences is preferably a sequence complement thereto according to the base pairing of nucleic acid molecules well known to the skilled person. For example, the bases A and G undergo base pairing with the bases T and U or C, resp. and visa versa. Modifications of the bases can influence the base-pairing partner.

[100909] The nucleic acid molecule of the invention comprises a nucleotide sequence which is at least about 30%, 35%, 40%, 45%, 50%, 55%, 60%, 65%, 70%, 75%, 80%, 90%, 95%, 96%, 97%, 98%, 99%, 99.5% or more homologous to a nucleotide sequence shown in Table I, application no. 19, columns 5 or 8, preferably the coding region thereof, homologs or fragments thereof, and preferably has above-mentioned activity, in particular conferring the production or the increased production of the fine chemical bis-glyceryl phosphate (polar fraction), glycerol (lipid fraction), glycerol (polar fraction), glycerol phosphate (lipid fraction), glycerol-3-phosphate (polar fraction), or triacylglycerol, respectively, after increasing the activity or an activity of a gene as shown in the respective line in Table I or of a gene product, e.g. as shown in the respective line in Table II, application no. 19, column 5 or 8, by for example in one embodiment expression either in the cytosol or in an organelle such as a plastid or mitochondria or both, preferably in a plastid, or in another embodiment non-targeted or targeted.

[100910] The nucleic acid molecule of the invention comprises a nucleotide sequence or molecule which hybridizes, preferably hybridizes under stringent conditions as defined herein, to one of the nucleic acid molecule shown in the respective line in Table I, preferably Table IB, application no. 19, columns 5 or 8, preferably the coding region thereof, fragments or homologs thereof, and encodes a protein having above-mentioned activity, e.g. conferring the production or the increased production of the respective fine chemical bis-glyceryl phosphate (polar fraction), glycerol (lipid fraction), glycerol (polar fraction), glycerol phosphate (lipid fraction), glycerol-3-phosphate (polar fraction), or triacylglycerol as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted, and optionally, the activity thereof is selected from the group consisting of (DL)-glycerol-3-phosphatase, 2-C-methyl-D-erythritol 4-phosphate cytidylyltransferase, 3oxoacyl-[acyl-carrier-protein] synthase, 49747384_SOYBEAN-protein, ABC transporter permease protein, acetyl CoA carboxylase, acid phosphatase precursor, acid shock protein, acyl transferase, adenylate kinase, alcohol dehydrogenase, amino-acid acetyltransferase, arginine decarboxylase, argininosuccinate synthetase, aromatic acid decarboxylase, At1g23110-protein, At1g67340-protein, ATP-binding component of a transport system, ATP-dependent RNA helicase, b0050-protein, b0518-protein, b0970-protein, b1003-protein, b1234-protein, b1330-protein, b1522-protein, b1556-protein, b1583-protein, b1670-protein, b1672-protein, b1703-protein, B1795-protein, b1837-protein, b2032-protein, b2099-protein, b2139-protein, b2399-protein, b2474-protein, b2613-protein, b2673-protein, b3083-protein, b3160-protein, b3777-protein, b3791-protein, b3989-protein, b4029-protein, b4050-protein, beta-hydroxylase, biotin synthase, calcium-dependent protein kinase, carbamoyl-phosphate synthase subunit, carbohydrate kinase, cardiolipin synthase, CBL-interacting protein kinase, CDP-diacylglycerol-glycerol-3-phosphate 3-phosphatidyltransferase, cell division control protein, cell division protein, coproporphyrinogen III oxidase, cyclin, cyclin D , dihydrolipoamide dehydrogenase, DnaJ-like chaperone, Elongation factor Tu, enterobacterial common antigen polymerase, ethanolamine utilization protein, eukaryotic translation initiation factor 5, Fe—S subunit of oxidoreductase, FKBP-type peptidyl-prolyl cis-trans isomerase (rotamase), glutamate 5-kinase, glutaredoxin, Glycine cleavage system T aminomethyltransferase, GM02LC17485-protein, heat shock protein Hsp40, heat shock transcription factor, HesB/YadR/YfhF family protein, high-affinity branched-chain amino acid transport protein, hydrolase, imidazole glycerol phosphate synthase subunit, ion-transport protein, lipoprotein precursor, L-serine dehydratase, lysozyme, lysyl-tRNA synthetase, metal-dependent hydrolase, methylglyoxal synthase, methyltransferase, Mg-protoporphyrin IX monomethyl ester oxidative cyclase 66 kD subunit, mitochondrial processing protease, mitogen-activated protein kinase, molecular chaperone portein, monothiol glutaredoxin, murein transglycosylase, NADH dehydrogenase, NADH-quinone oxidoreductase subunit, nitrate/nitrite transport protein, oxidoreductase, phenylacetic acid degradation protein, phosphate import ATP-binding protein, phosphatidylinositol 3- and 4-kinase family protein, phosphoglyceromutase, phosphotransferase, phosphotransferase system component, photosystem I reaction centre subunit, polygalacturonase, precorrin-8X methylmutase, protein phosphatase, purine-nucleoside phosphorylase, putative transport system permease protein, putrescine transport system permease protein, pyruvate kinase, recombinase A, sec-independent protein translocase, serine protease, sodium/proton antiporter, ThiF family protein, threonine dehydratase, threonine synthase, threonine/serine transporter, transcription factor, transcriptional regulator, transcriptional regulator protein, trehalose-phosphatase, TTC1386-protein, vacuolar sorting protein, YAL013W-protein, ycr102c-protein, ydr273w-protein, YFL019C-protein, YHR202W-protein, YIL083C-protein, YJL062W-A-protein, YLR099C-protein, ylr326w-protein, YPL162C.protein, and Zm_{—}4842_BE510522-protein, respectively.

[100911] Moreover, the nucleic acid molecule of the invention can comprise only a portion of the coding region of one of the nucleic acid molecules depicted in Table I, application no. 19, columns 5 or 8, for example a fragment which can be used as a probe or primer or a fragment encoding a biologically active portion of the polypeptide of the present invention or of a polypeptide used in the process of the present invention, i.e. having above-mentioned activity, e.g. conferring the production or the increased production of the respective fine chemical bis-glyceryl phosphate (polar fraction), glycerol (lipid fraction), glycerol (polar fraction), glycerol phosphate (lipid fraction), glycerol-3-phosphate (polar fraction), or triacylglycerol as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted. The nucleotide sequences determined from the cloning of the present protein-according-to-the-invention-encoding gene allows for the generation of probes and primers designed for use in identifying and/or cloning its homologues from other cell types and organisms. The probe/primer typically comprises a substantially purified oligonucleotide. The oligonucleotide typically comprises a region of nucleotide sequence that hybridizes under stringent conditions to at least about 12, 15 preferably about 20 or 25, more preferably about 40, 50 or 75 consecutive nucleotides of a sense strand of one of the sequences set forth, e.g., in the respective line in Table I, application no. 19, column 5 or 8, preferably the coding region thereof, or a homolog or a fragment thereof, an anti-sense sequence of one of the sequences, e.g., set forth in the respective line in Table I, application no. 19, columns 5 or 8, preferably the coding region thereof, or a homolog or a fragment thereof or naturally occurring mutants thereof. Primers based on a nucleic acid molecule of invention can be used in PCR reactions to clone homologs of the polypeptide of the invention or of the polypeptide used in the process of the invention, e.g. as the primers described in the examples of the present invention, e.g. as shown in the examples. A PCR with the primers shown in the respective line in Table III, column 8 will result in a fragment of the gene product as shown in Table II, application no. 19, column 5.

[100912] Primer sets are interchangeable. The person skilled in the art knows how to design and combine said primers to result in the desired product, e.g. in a full length clone or a partial sequence. Probes based on the sequences of the nucleic acid molecule of the invention or used in the process of the present invention can be used to detect transcripts or genomic sequences encoding the same or homologous proteins. The probe can further comprise a label group attached thereto, e.g. the label group can be a radioisotope, a fluorescent compound, an enzyme, or an enzyme co-factor. Such probes can be used as a part of a genomic marker test kit for identifying cells which express a polypeptide of the invention or used in the process of the present invention, such as by measuring a level of an encoding nucleic acid molecule in a sample of cells, e.g. detecting mRNA levels or determining, whether a genomic gene comprising the sequence of the polynucleotide of the invention or used in the processes of the present invention has been mutated or deleted.

[100913] As the expression of a respective nucleic acids of the invention is related to the synthesis of the respective fine chemical bis-glyceryl phosphate (polar fraction), glycerol (lipid fraction), glycerol (polar fraction), glycerol phosphate (lipid fraction), glycerol-3-phosphate (polar fraction), or triacylglycerol its function as a probe extends to the detection of microorganisms, plant tissues, plants, plant variets, plant ecotypes or plant genera with varying capability or potential for synthesis of the respective fine chemical bis-glyceryl phosphate (polar fraction), glycerol (lipid fraction), glycerol (polar fraction), glycerol phosphate (lipid fraction), glycerol-3-phosphate (polar fraction), or triacylglycerol. Therefore in one embodiment the present invention relates to a method for analyzing the capability or potential of a plant tissue, a plant, a plant variety or plant ecotype to produce the fine chemical bis-glyceryl phosphate (polar fraction), glycerol (lipid fraction), glycerol (polar fraction), glycerol phosphate (lipid fraction), glycerol-3-phosphate (polar fraction), or triacylglycerol by using the nucleic acid of the invention or parts thereof as a probe to detect the amount of the nucleic acid of the invention in the non-human organism or a part thereof in comparison to another non-human organism.

[100914] The nucleic acid molecule of the invention encodes a polypeptide or portion thereof which includes an amino acid sequence which is sufficiently homologous to the amino acid sequence shown in respective line in Table II, application no. 19, columns 5 or 8 such that the protein or portion thereof maintains the ability to confer the production or the increased production of the respective fine chemical bis-glyceryl phosphate (polar fraction), glycerol (lipid fraction), glycerol (polar fraction), glycerol phosphate (lipid fraction), glycerol-3-phosphate (polar fraction), or triacylglycerol as compared to a corresponding, e.g. non-transformed, wild type non-human organism, like a microorganism or a plant cell, plant or part thereof, in particular increasing the activity as mentioned above or as described in the examples in microorganisms or plants.

[100915] As used herein, the language “sufficiently homologous” refers to proteins or portions thereof which have amino acid sequences which include a minimum number of identical or equivalent amino acids (e.g. an amino acid which has a similar side chain as an amino acid in one of the sequences of the polypeptide of the present invention) to an amino acid sequence shown in the respective line in Table II, application no. 19, columns 5 or 8 such that the protein or portion thereof is able to confer the production or the increased production of the respective fine chemical bis-glyceryl phosphate (polar fraction), glycerol (lipid fraction), glycerol (polar fraction), glycerol phosphate (lipid fraction), glycerol-3-phosphate (polar fraction), or triacylglycerol as compared to a corresponding, e.g. non-transformed, wild type non-human organism, like a microorganism or a plant cell, plant or part thereof. For example having the activity of a protein as shown in the respective line in Table II, application no. 19, column 3 and as described herein.

[100916] In an embodiment of the present invention, the nucleic acid molecule of the present invention comprises a nucleic acid molecule that encodes a portion of the protein of the present invention. The protein is at least about 30%, 35%, 40%, 45% 50%, 55%, 60%, 65%, 70%, 75%, 80%, 85%, 90%, 91%, 92%, 93%, 94%, 95%, 96%, 97%, 98%, 99%, 99.5% or more homologous to an entire amino acid sequence shown in the respective line of Table II, application no. 19, column 5 or 8 and has the above-mentioned activity, e.g. conferring the production or the increased production of the respective fine chemical bis-glyceryl phosphate (polar fraction), glycerol (lipid fraction), glycerol (polar fraction), glycerol phosphate (lipid fraction), glycerol-3-phosphate (polar fraction), or triacylglycerol as compared to a corresponding, e.g. non-transformed, wild type non-human organism, like a microorganism or a plant cell, plant or part thereof by, for example in a embodiment expression either in the cytosol or in an organelle such as a plastid or mitochondria or both, preferably in a plastid, or in another embodiment by targeted or non-targeted expression.

[100917] for the disclosure of this paragraph see [0338.1.7.7] above

[100918] for the disclosure of this paragraph see [0339.1.7.7] above

[100919] The invention further relates to nucleic acid molecules or methods using said nucleic acid molecules that differ from one of the nucleotide sequences shown in the respective line in Table I, application no. 19, columns 5 or 8, preferably the coding region thereof, or fragments or homologs thereof, due to degeneracy of the genetic code but encode a polypeptide of the present invention, in particular a polypeptide having the above-mentioned activity, e.g. as those polypeptides depicted in the respective line in Table II, application no. 19, columns 5 or 8, fragments or homologs thereof. Advantageously, the nucleic acid molecule of the invention comprises, or in an other embodiment has, a nucleotide sequence encoding a protein comprising, or in an other embodiment having, an amino acid sequence shown in the respective line in Table II, application no. 19, columns 5 or 8, fragments or homologs thereof. In a further embodiment, the nucleic acid molecule of the invention encodes a full length protein which is substantially homologous to an amino acid sequence shown in the respective line in Table II, application no. 19, columns 5 or 8, or homologs thereof. However, in an embodiment, the nucleic acid molecule of the present invention does not consist of the sequence shown in the respective line in Table I, preferably Table IA, application no. 19, columns 5 or 8, preferably the coding region thereof, but the functional homologs thereof.

[100920] [0341.1.7.19] to [0343.1.7.19] for the disclosure of these paragraphs see [0341.1.7.7] to [0343.1.7.7] above

[100921] Accordingly, in another embodiment, a nucleic acid molecule of the invention is at least 15, 20, 25 or 30 nucleotides in length. Preferably, it hybridizes under stringent conditions to a nucleic acid molecule comprising a nucleotide sequence of the nucleic acid molecule of the present invention or used in the process of the present invention, e.g. comprising the sequence shown in the respective line in Table I, application no. 19, columns 5 or 8, preferably the coding region thereof. In another embodiment the nucleic acid molecule is preferably at least 20, 30, 50, 100, 250 or more nucleotides in length.

[100922] for the disclosure of this paragraph see [0345.1.7.7] above

[100923] Preferably, a nucleic acid molecule of the invention that hybridizes under stringent conditions to a sequence shown in the respective line in Table I, application no. 19, columns 5 or 8, preferably the coding region thereof, corresponds to a naturally-occurring nucleic acid molecule of the invention. As used herein, a “naturally-occurring” nucleic acid molecule refers to a RNA or DNA molecule having a nucleotide sequence that occurs in nature (e.g. encodes a natural protein). Preferably, the nucleic acid molecule encodes a natural protein having above-mentioned activity, e.g. conferring the production or the increased production of the respective fine chemical bis-glyceryl phosphate (polar fraction), glycerol (lipid fraction), glycerol (polar fraction), glycerol phosphate (lipid fraction), glycerol-3-phosphate (polar fraction), or triacylglycerol as compared to a corresponding, e.g. non-transformed, wild type non-human organism, like a microorganism or a plant cell, plant or part thereof after increasing the expression or activity thereof or the activity of a protein of the invention or used in the process of the invention, in an embodiment for example expression either in the cytosol or in an organelle such as a plastid or mitochondria, preferably in plastids, or, in another embodiment by targeted or non-targeted expression.

[100924] for the disclosure of this paragraph see [0347.1.7.7] above

[100925] For example, nucleotide substitutions leading to amino acid substitutions at “non-essential” amino acid residues can be made in a sequence of the nucleic acid molecule of the invention or used in the process of the invention, e.g. shown in the respective line in Table I, application no. 19, columns 5 or 8, preferably the coding region thereof, or fragments or homologs thereof.

[100926] for the disclosure of this paragraph see [0349.1.7.7] above

[100927] for the disclosure of this paragraph see [0350.1.7.7] above

[100928] Accordingly, the invention relates to nucleic acid molecules encoding a polypeptide having above-mentioned activity, in a non-human organisms or parts thereof by for example expression either in the cytosol or in an organelle such as a plastid or mitochondria or both, preferably in plastids, that contain changes in amino acid residues that are not essential for said activity. Such polypeptides differ in amino acid sequence from a sequence depicted in the respective line in Table II, especially Table IIA, application no. 19, columns 5 or 8, but retain said activity described herein. The nucleic acid molecule can comprise a nucleotide sequence encoding a polypeptide, wherein the polypeptide comprises an amino acid sequence at least about 50% identical to an amino acid sequence shown in the respective line in Table II, application no. 19, columns 5 or 8, and is capable of conferring the production or the increased production of the respective fine chemical bis-glyceryl phosphate (polar fraction), glycerol (lipid fraction), glycerol (polar fraction), glycerol phosphate (lipid fraction), glycerol-3-phosphate (polar fraction), or triacylglycerol as compared to a corresponding, e.g. non-transformed, wild type non-human organism, like a microorganism or a plant cell, plant or part thereof, after increasing its activity for example in an embodiment by expression either in the cytosol or in an organelle such as a plastid or mitochondria or both, preferably in plastids, or, in another embodiment by targeted or non-targeted expression. Preferably, the protein encoded by the nucleic acid molecule is at least about 60%, 70%, 80%, 90%, 95%, 96%, 97%, 98%, 99% or 99.5% identical to the sequence shown in the respective line in Table II, application no. 19, columns 5 or 8.

[100929] [0352.1.7.19] to [0357.1.7.19] for the disclosure of these paragraphs see [0352.1.7.7] to [0357.1.7.7] above

[100930] Functional equivalents of the nucleic acid molecules according to the present invention and/or used in the process according to the present invention represent an embodiment of homologs. Functional equivalents derived from the nucleic acid molecules as shown in the respective line in Table I, application no. 19, columns 5 or 8, preferably the coding region thereof, according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the nucleic acid molecules as shown in the respective line in Table I, application no. 19, columns 5 or 8, preferably the coding region thereof, according to the invention and encode polypeptides having essentially the same properties as the polypeptide as shown in the respective line in Table II, application no. 19, columns 5 or 8.

[100931] Functional equivalents derived from one of the polypeptides as shown in the respective line in Table II, application no. 19, columns 5 or 8 according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the polypeptides as shown in the respective line in Table II, application no. 19, columns 5 or 8 according to the invention and having essentially the same properties as the polypeptide as shown in the respective line in Table II, application no. 19, columns 5 or 8.

[100932] for the disclosure of this paragraph see [0359.1.7.7] above

[100933] A nucleic acid molecule encoding a homolog to a polypeptide depicted in the respective line in Table II, application no. 19, columns 5 or 8 can be created by introducing one or more nucleotide substitutions, additions or deletions into the nucleic acid molecule of the present invention, in particular of Table I, application no. 19, columns 5 or 8, in the respective line, such that one or more amino acid substitutions, additions or deletions are introduced into the encoded protein. Mutations can be introduced into the nucleic acid molecules as depicted in the respective line in Table I, application no. 19, columns 5 or 8, preferably the coding region thereof, by standard techniques, such as site-directed mutagenesis, PCR-mediated mutagenesis or other methods known to the person skilled in the art.

[100934] [0361.1.7.19] to [0363.1.7.19] for the disclosure of these paragraphs see [0361.1.7.7] to [0363.1.7.7] above

[100935] Homologues of the nucleic acid sequences used, with the sequences shown in the respective line in Table I, application no. 19, columns 5 or 8, preferably the coding region thereof, comprise also allelic variants with at least approximately 30%, 35%, 40%, 45%, 50%, 55%, 60%, 65%, 70%, 75%, 80%, 85%, 90%, 91%, 92%, 93%, 94% 95%. 96%, 97%, 98%, 99%, 99.5% or more % homology with one of the nucleotide sequences shown or the above-mentioned derived nucleic acid sequences or their homologues or derivatives or parts of these. Allelic variants encompass in particular functional variants which can be obtained by deletion, insertion or substitution of nucleotides from the sequences shown in the respective line, preferably in Table I, columns 5 or 8, preferably the coding region thereof, or from the derived nucleic acid sequences, the intention being, however, that the enzymatic activity or the biological activity of the resulting proteins synthesized is advantageously retained or increased.

[100936] In one embodiment of the present invention, the nucleic acid molecule of the invention or used in the process of the invention comprises the sequences shown in the respective line in any of the Table I, application no. 19, columns 5 or 8, preferably the coding region thereof, fragments or homologs thereof. It is preferred that the nucleic acid molecule comprises as little as possible other nucleotides not shown in the respective line in any one of Table I, application no. 19, columns 5 or 8, preferably the coding region thereof, fragments or homologs thereof. In one embodiment, the nucleic acid molecule comprises less than 500, 400, 300, 200, 100, 90, 80, 70, 60, 50 or 40 further nucleotides. In a further embodiment, the nucleic acid molecule comprises less than 30, 20 or 10 further nucleotides. In one embodiment, the nucleic acid molecule used in the process of the invention is identical to the sequences shown in the respective line in Table I, application no. 19, columns 5 or 8, preferably the coding region thereof.

[100937] [0366.1.19.19] Also preferred is that the nucleic acid molecule used in the process of the invention encodes a polypeptide comprising the sequence shown in the respective line in Table II, application no. 19, columns 5 or 8, fragments or homologs thereof. In one embodiment, the nucleic acid molecule encodes less than 150, 130, 100, 80, 60, 50, 40 or 30 further amino acids. In a further embodiment, the encoded polypeptide comprises less than 20, 15, 10, 9, 8, 7, 6 or 5 further amino acids. In one embodiment used in the inventive process, the encoded polypeptide is identical to the sequences shown in the respective line in Table II, application no. 19, columns 5 or 8.

[100938] In one embodiment, the nucleic acid molecule of the invention or used in the process encodes a polypeptide comprising the sequence shown in the respective line in Table II, application no. 19, columns 5 or 8, or fragments or homologs thereof, and comprises less than 100 further nucleotides. In a further embodiment, said nucleic acid molecule comprises less than 30 further nucleotides. In one embodiment, the nucleic acid molecule used in the process is identical to a coding sequence of the sequences shown in the respective line in Table I, application no. 19, columns 5 or 8.

[100939] Polypeptides (=proteins), which still have the essential biological or enzymatic activity of the polypeptide of the present invention conferring the production or the increased production of the respective fine chemical bis-glyceryl phosphate (polar fraction), glycerol (lipid fraction), glycerol (polar fraction), glycerol phosphate (lipid fraction), glycerol-3-phosphate (polar fraction), or triacylglycerol as compared to a corresponding, e.g. non-transformed, wild type non-human organism, like a microorganism or a plant cell, plant or part thereof, i.e. whose activity is essentially not reduced, are polypeptides with at least 10%, 20%, 30%, 40%, 50%, 60%, 70%, 80%, 90%, 95% or more of the wild type biological activity or enzymatic activity, advantageously, the activity is essentially not reduced in comparison with the activity of a polypeptide shown in the respective line in Table II, application no. 19, columns 5 or 8 expressed under identical conditions.

[100940] [0369.1.19.19] Homologues of nucleic acid molecules shown in the respective line in Table I, application no. 19, columns 5 or 8, preferably the coding region thereof, or fragments thereof, or of the derived sequences shown in the respective line in Table II, application no. 19, columns 5 or 8, or fragments thereof, also mean truncated sequences, cDNA, single-stranded DNA or RNA of the coding and noncoding DNA sequence. Homologues of said sequences are also understood as meaning derivatives, which comprise noncoding regions such as, for example, UTRs, introns, terminators, enhancers or promoter variants. The promoters upstream of the nucleotide sequences stated can be modified by one or more nucleotide substitution(s), insertion(s) and/or deletion(s) without, however, interfering with the functionality or activity either of the promoters, the open reading frame (=ORF) or with the 3′-regulatory region such as terminators or other 3′-regulatory regions, which are far away from the ORF. It is furthermore possible that the activity of the promoters is increased by modification of their sequence, or that they are replaced completely by more active promoters, even promoters from heterologous organisms.

[100941] Appropriate promoters are known to the person skilled in the art and are mentioned herein below.

[100942] [0370.1.7.19] to [0379.1.7.19] for the disclosure of these paragraphs see [0370.1.7.7] to [0379.1.7.7] above

[100943] Moreover, a native polypeptide conferring the increase of the respective fine chemical bis-glyceryl phosphate (polar fraction), glycerol (lipid fraction), glycerol (polar fraction), glycerol phosphate (lipid fraction), glycerol-3-phosphate (polar fraction), or triacylglycerol in a non-human organism or a part thereof can be isolated from cells (e.g., endothelial cells), for example using the antibody of the present invention as described below, in particular, an antibody against proteins having (DL)-glycerol-3-phosphatase, 2-C-methyl-D-erythritol 4-phosphate cytidylyltransferase, 3-oxoacyl-[acyl-carrier-protein] synthase, 49747384_SOYBEAN-protein, ABC transporter permease protein, acetyl CoA carboxylase, acid phosphatase precursor, acid shock protein, acyl transferase, adenylate kinase, alcohol dehydrogenase, amino-acid acetyltransferase, arginine decarboxylase, argininosuccinate synthetase, aromatic acid decarboxylase, At1g23110-protein, At1g67340-protein, ATP-binding component of a transport system, ATP-dependent RNA helicase, b0050-protein, b0518-protein, b0970-protein, b1003-protein, b1234-protein, b1330-protein, b1522-protein, b1556-protein, b1583-protein, b1670-protein, b1672-protein, b1703-protein, B1795-protein, b1837-protein, b2032-protein, b2099-protein, b2139-protein, b2399-protein, b2474-protein, b2613-protein, b2673-protein, b3083-protein, b3160-protein, b3777-protein, b3791-protein, b3989-protein, b4029-protein, b4050-protein, beta-hydroxylase, biotin synthase, calcium-dependent protein kinase, carbamoyl-phosphate synthase subunit, carbohydrate kinase, cardiolipin synthase, CBL-interacting protein kinase, CDP-diacylglycerol-glycerol-3-phosphate 3-phosphatidyltransferase, cell division control protein, cell division protein, coproporphyrinogen III oxidase, cyclin, cyclin D , dihydrolipoamide dehydrogenase, DnaJ-like chaperone, Elongation factor Tu, enterobacterial common antigen polymerase, ethanolamine utilization protein, eukaryotic translation initiation factor 5, Fe—S subunit of oxidoreductase, FKBP-type peptidyl-prolyl cis-trans isomerase (rotamase), glutamate 5-kinase, glutaredoxin, Glycine cleavage system T aminomethyltransferase, GM02LC17485-protein, heat shock protein Hsp40, heat shock transcription factor, HesB/YadR/YfhF family protein, high-affinity branched-chain amino acid transport protein, hydrolase, imidazole glycerol phosphate synthase subunit, ion-transport protein, lipoprotein precursor, L-serine dehydratase, lysozyme, lysyl-tRNA synthetase, metal-dependent hydrolase, methylglyoxal synthase, methyltransferase, Mg-protoporphyrin IX monomethyl ester oxidative cyclase 66 kD subunit, mitochondrial processing protease, mitogen-activated protein kinase, molecular chaperone portein, monothiol glutaredoxin, murein transglycosylase, NADH dehydrogenase, NADH-quinone oxidoreductase subunit, nitrate/nitrite transport protein, oxidoreductase, phenylacetic acid degradation protein, phosphate import ATP-binding protein, phosphatidylinositol 3- and 4-kinase family protein, phosphoglyceromutase, phosphotransferase, phosphotransferase system component, photosystem I reaction centre subunit, polygalacturonase, precorrin-8X methylmutase, protein phosphatase, purine-nucleoside phosphorylase, putative transport system permease protein, putrescine transport system permease protein, pyruvate kinase, recombinase A, secindependent protein translocase, serine protease, sodium/proton antiporter, ThiF family protein, threonine dehydratase, threonine synthase, threonine/serine transporter, transcription factor, transcriptional regulator, transcriptional regulator protein, trehalose-phosphatase, TTC1386-protein, vacuolar sorting protein, YAL013W-protein, ycr102c-protein, ydr273w-protein, YFL019C-protein, YHR202W-protein, YIL083C-protein, YJL062W-A-protein, YLR099C-protein, ylr326w-protein, YPL162C.protein, or Zm_{—}4842_BE510522-protein activity, respectively, or an antibody against polypeptides as shown in the respective line in Table II, application no. 19, columns 5 or 8, or fragments or homologs thereof which can be produced by standard techniques utilizing the polypeptid of the present invention or fragment thereof, i.e., the polypeptide of this invention (FCRP). Preferred are monoclonal antibodies.

[100944] for the disclosure of this paragraph see [0381.1.7.7] above

[100945] In one embodiment, the present invention relates to a polypeptide as depicted in the respective line in Table II, especially in Table IIA or especially in Table IIB, application no. 19, columns 5 or 8, or fragment or homolog thereof or as coded by the nucleic acid molecule shown in the respective line in Table I, especially Table IA or especially Table IB, application no. 19, columns 5 or 8, preferably the coding region thereof, or fragment or homolog thereof.

[100946] In an embodiment, the present invention relates to a polypeptide comprising or consisting of a consensus sequence shown in the respective line in Table IV, application no. 19, column 8 or at least one polypeptide motif shown in the respective line in Table IV, application no. 19, column 8.

[100947] In an embodiment thereof, the present invention relates to a polypeptide comprising or consisting of a consensus sequence shown in the respective line in Table IV, application no. 19, column 8 or comprising at least one polypeptide motif shown in the respective line in Table IV, application no. 19, column 8, whereby not more than 20, 15, 10, 9, 8, 7, 6, 5, 4, 3, 2, or 1, or 0 of the amino acids can be replaced by any amino acid.

[100948] In another embodiment thereof, the present invention relates to a polypeptide comprising or consisting of a consensus sequence shown in the respective line in Table IV, application no. 19, column 8 or comprising at least one polypeptide motif shown in the respective line in Table IV, application no. 19, column 8, whereby not more than 15%, 10%, 5%, 4%, 3%, 2% or1% or 0% of the amino acids defined as distinct specific amino acids are/is replaced by another amino acid.

[100949] In another embodiment thereof, the present invention relates to a polypeptide comprising or consisting of a consensus sequence shown in the respective line in Table IV, application no. 19, column 8 or comprising at least one polypeptide motif shown in the respective line in Table IV, application no. 19, column 8, whereby not more than 20, 15, 10, 9, 8, 7, 6, 5, 4, 3, 2, or 1, or 0 amino acids are inserted into the consensus sequence or polypeptide motif.

[100950] In an embodiment of the present invention, the polypeptide of the present invention comprises or consists of a consensus sequence or at least one polypeptide motif shown in the respective line in Table IV, application no. 19, column 8, whereby said polypeptide distinguishes over the sequence depicted in the respective line in Table II, application no. 19, columns 5 or 8 by one or more amino acids. In one embodiment, said polypeptide of the invention distinguishes over the sequence shown in the respective line in Table II, application no. 19, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 19, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in the respective line in Table II, application no. 19, columns 5 and 8.

[100951] In an embodiment, the invention relates to a polypeptide conferring an increase in the respective fine chemical bis-glyceryl phosphate (polar fraction), glycerol (lipid fraction), glycerol (polar fraction), glycerol phosphate (lipid fraction), glycerol-3-phosphate (polar fraction), or triacylglycerol in a non-human organism, especially a microorganism or a plant, or a part thereof, being encoded by the nucleic acid molecule of the invention or used in the process of the invention and having a sequence which distinguishes over the sequence as shown in the respective line in Table II, application no. 19, columns 5 or 8 by one or more amino acids (but not exceeding 5%, preferably 4%, 3%, 2%, 1%, 0.5%, 0.2% or 0.1%). In an embodiment, said polypeptide of the invention does not comprise or consist of the sequence shown in the respective line in Table II, application no. 19, columns 5 or 8. In an embodiment, said polypeptide of the present invention is less than 100%, 99.999%, 99.99%, 99.9% or 99% identical. In one embodiment, said polypeptide which differs at least in one or more amino acids (but not exceeding 5%, preferably 4%, 3%, 2%, 1%, 0.5%, 0.2% or 0.1%) from the polypeptide shown in the respective line in Table II, application no. 19, columns 5 and 8 does not comprise a protein of the sequence shown in the respective line in Table II A and/or II B, application no. 19, columns 5 or 8.

[100952] In an embodiment, the present invention relates to a polypeptide having the activity of the protein as shown in the respective line in Table II, application no. 19, column 3, which distinguishes over the sequence depicted in the respective line in Table II, application no. 19, columns 5 or 8 by one or more amino acids, preferably by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 19, columns 5 and 8 by not more than 80%, 70%, 60%, 50%, 40%, 30%, 20%, 10%, 5%, 4%, 3%, 2%, 1% or 0.5%. In another embodiment the polypeptide of the invention takes the form of a preprotein consisting of a plastidial or mitochondrial transit peptide joint to a polypeptide having the activity of the protein as shown in Table II, column 3, from which the transit peptide is preferably cleaved off upon transport of the preprotein into the organelle for example into the plastid or mitochondrion. In another embodiment the polypeptide of the invention takes not the form of a preprotein.

[100953] for the disclosure of this paragraph see [0390.1.7.7] above

[100954] for the disclosure of this paragraph see [0391.1.7.7] above

[100955] A polypeptide of the invention can participate in the process of the present invention. The polypeptide or a portion thereof comprises preferably an amino acid sequence, which is sufficiently homologous to an amino acid sequence shown in the respective line in Table II A and/or II B, application no. 19, columns 5 or 8.

[100956] Further, the polypeptide can have an amino acid sequence which is encoded by a nucleotide sequence which hybridizes, preferably hybridizes under stringent conditions as described above, to a nucleotide sequence of the nucleic acid molecule of the present invention. Accordingly, the polypeptide has an amino acid sequence which is encoded by a nucleotide sequence that is at least about 35%, 40%, 45%, 50%, 55%, 60%, 65%, 70%, 75%, 80%, 85%, 90%, 91%, 92%, 93%, 94%, 95%, 96%, 97%, 98%, 99%, 99.5% or more homologous to one of the amino acid sequences as shown in the respective line in Table II A and/or II B, application no. 19, columns 5 or 8, or fragments thereof. The preferred polypeptide of the present invention preferably possesses at least one of the activities according to the invention and described herein. A preferred polypeptide of the present invention includes an amino acid sequence encoded by a nucleotide sequence which hybridizes, preferably hybridizes under stringent conditions, to a nucleotide sequence shown in the respective line in Table I A and/or I B, application no. 19, columns 5 or 8, preferably the coding region thereof, or fragments thereto or which is homologous thereto, as defined above

[100957] The polypeptide of the present invention can vary from the sequences shown in the respective line in Table II A and/or II B, application no. 19, columns 5 or 8 in amino acid sequence due to natural variation or mutagenesis, as described in detail herein. Accordingly, the polypeptide comprise an amino acid sequence which is at least about 35%, 40%, 45%, 50%, 55%, 60%, 65%, 70%, 75%, 80%, 85% or 90, 91%, 92%, 93%, 94%, 95%, 96%, 97%, 98%, 99%, 99.5% or more homologous to an entire amino acid sequence shown in the respective line in Table II A and/or II B, application no. 19, columns 5 or 8.

[100958] Biologically active portions of a polypeptide of the present invention include peptides comprising amino acid sequences derived from the amino acid sequence of the polypeptide of the present invention or used in the process of the present invention, e.g., the amino acid sequence shown in the respective line in Table II, application no. 19, columns 5 or 8 or the amino acid sequence of a protein homologous thereto, which include fewer amino acids than a full length polypeptide of the present invention or used in the process of the present invention or the full length protein which is homologous to a polypeptide of the present invention or used in the process of the present invention depicted herein, and exhibit at least one activity of a polypeptide of the present invention or used in the process of the present invention.

[100959] for the disclosure of this paragraph see [0396.1.7.7] above

[100960] Manipulation of the nucleic acid molecule of the invention may result in the production of a protein having differences from the protein as shown in the respective line in Table II, application no. 19, column 5 or 8. Differences shall mean at least one amino acid different from the sequences as shown in the respective line in Table II, application no. 19, column 5 or 8, preferably at least 2, 3, 4, 5, 6, 7, 8, 9 or 10 amino acids (especially but not exceeding 0.1%, preferably 0.2%, 0.5%, 1%, 2%, 3% 4% or 5%), more preferably at least 15, 20, 25, 30, 35, 40, 45 or 50 amino acids (especially but not exceeding 0.5%, preferably 1%, 2%, 3% 4%, 5%, 10% or 25%) different from the sequences as shown in the respective line in Table II, application no. 19, column 5 or 8. These proteins may be improved in efficiency or activity, or may be more stable and therefore present in greater numbers in the cell as compared to the wild-type cell.

[100961] Any mutagenesis strategies for the polypeptide of the present invention or the polypeptide used in the process of the present invention to result in increasing said activity are not meant to be limiting; variations on these strategies will be readily apparent to one skilled in the art. Using such strategies, and incorporating the mechanisms disclosed herein, the nucleic acid molecule and polypeptide of the invention may be utilized to generate algae, ciliates, fungi, other microorganisms like C. glutamicum or plants or parts thereof, expressing proteins as shown in the respective line in Table II, application no. 19, column 5 or 8, or mutated proteins thereof. The nucleic acid molecules and polypeptide molecules of the invention are expressed such that the yield, production, and/or efficiency of production of a desired compound is improved.

[100962] [0399.1.7.19] to [0401.1.7.19] for the disclosure of these paragraphs see [0399.1.7.7] to [0401.1.7.7] above

[100963] In one embodiment, a protein (=polypeptide) as shown in the respective line in Table II, application no. 19, column 5 or 8 refers to a polypeptide having an amino acid sequence corresponding to the polypeptide of the invention, especially to a polypeptide as shown in Table II, application no. 19, columns 5 or 8 or homologs thereof, or used in the process of the invention, whereas a “non- inventive protein or polypeptide” or “other polypeptide” refers to a polypeptide having an amino acid sequence corresponding to a protein which is not substantially homologous to a polypeptide of the invention, preferably which is not substantially homologous to a polypeptide or protein as shown in the respective line in Table II, application no. 19, column 5 or 8 or homologs thereof, e.g., a protein which does not confer the activity described herein and which is derived from the same or a different organism.

[100964] [0403.1.7.19] to [0409.1.7.19] for the disclosure of these paragraphs see [0403.1.7.7] to [0409.1.7.7] above

[100965] As the nucleic acid molecules of the invention and/or the expression thereof is related to the synthesis of the respective fine chemical bis-glyceryl phosphate (polar fraction), glycerol (lipid fraction), glycerol (polar fraction), glycerol phosphate (lipid fraction), glycerol-3-phosphate (polar fraction), or triacylglycerol its function as a probe extends to the detection of microorganisms, plant tissues, plants, plant variets, plant ecotypes or plant genera with varying, advantageously increased, capability or potential for synthesis of the respective fine chemical bis-glyceryl phosphate (polar fraction), glycerol (lipid fraction), glycerol (polar fraction), glycerol phosphate (lipid fraction), glycerol-3-phosphate (polar fraction), or triacylglycerol. Therefore in one embodiment the present invention relates to a method for analyzing the capability or potential of a plant tissue, a plant, a plant variety or ecotype to produce the respective fine chemical bis-glyceryl phosphate (polar fraction), glycerol (lipid fraction), glycerol (polar fraction), glycerol phosphate (lipid fraction), glycerol-3-phosphate (polar fraction), or triacylglycerol by using the respective antibody of the invention as a probe to detect the amount of the polypeptide encoded by said nucleic acid molecule of the invention in a non-human organism in comparison to another organism.

[100966] Therefore in one embodiment the invention relates to a method to probe a non-human organism for its capability for the production of the respective fine chemical comprising the steps of
[100967] a) analyzing the sequence and/or the expression of the respective nucleic acid molecule of the invention in least two non-human organisms which differ in the capability to produce the respective fine chemical;
[100968] b) associate the different sequence and/or expression level of the nucleic acid molecules of the invention with their different capability to produce the respective fine chemical;
[100969] c) analyze the sequence and/or the expression of the nucleic acid molecule of the invention in at least a third non-human organism;
[100970] d) predict the capability of the at least third organism to produce the respective fine chemical based by comparing the sequence and/or expression of the nucleic acid of the invention to the sequences and/or expression levels of the nucleic acid molecule of the invention of the at least two organisms analyzed in step a.

[100971] [0411.1.7.19] to [0430.1.7.19] for the disclosure of these paragraphs see [0411.1.7.7] to [0430.1.7.7] above

[100972] Accordingly the present invention relates to any cell transgenic for any nucleic acid characterized as part of the invention, e.g. conferring the increase of the respective fine chemical bis-glyceryl phosphate (polar fraction), glycerol (lipid fraction), glycerol (polar fraction), glycerol phosphate (lipid fraction), glycerol-3-phosphate (polar fraction), or triacylglycerol in a cell or a non-human organism or a part thereof, e.g. the nucleic acid molecule of the invention, the nucleic acid construct of the invention, the vector of the invention, the expression cassette according to the invention, or a nucleic acid molecule encoding the polypeptide of the invention, e.g. encoding a polypeptide having an activity as the protein as shown in the respective line in Table II, application no. 19, column 3. Due to the above-mentioned activity the respective fine chemical bis-glyceryl phosphate (polar fraction), glycerol (lipid fraction), glycerol (polar fraction), glycerol phosphate (lipid fraction), glycerol-3-phosphate (polar fraction), or triacylglycerol content in a cell or a non-human organism is increased. For example, due to modulation or manipulation, the cellular activity is increased, in a preferred embodiment in organelles such as plastids or mitochondria, e.g. due to an increased expression or specific activity or specific targeting of the subject matters of the invention in a cell or a non-human organism or a part thereof especially in organelles such as plastids or mitochondria, or in another embodiment in the cytosol. Transgenic for a polypeptide having a protein or a protein activity means herein that due to modulation or manipulation of the genome, the activity of protein as shown in the respective line in Table II, application no. 19, column 3 or a protein as shown in the respective line in Table II, application no. 19, column 3-like activity is increased in the cell or non-human organism or part thereof, especially in organelles such as plastids or mitochondria, or especially in the cytosol. Examples are described above in context with the process of the invention.

[100973] for the disclosure of this paragraph see [0432.1.7.7] above

[100974] A naturally occurring expression cassette—for example the naturally occurring combination of the promoter of the gene encoding a protein as shown in the respective line in Table II, application no. 19, column 5 or 8, preferably the coding region thereof, or fragments or homologs thereof with the corresponding encoding gene—becomes a transgenic expression cassette when it is modified by non-natural, synthetic “artificial” methods such as, for example, mutagenesis. Such methods have been described (U.S. Pat. No. 5,565,350; WO 00/15815; also see above).

[100975] for the disclosure of this paragraph see [0434.1.7.7] above

[100976] for the disclosure of this paragraph see [0435.1.7.7] above

[100977] Transgenic plants comprising the respective fine chemical(s) synthesized in the process according to the invention can be marketed directly without isolation of the fine chemical synthesized. In the process according to the invention, plants are understood as meaning all plant parts, plant organs such as leaf, stalk, root, tubers or seeds or propagation material or harvested material or the intact plant. In this context, the seed encompasses all parts of the seed such as the seed coats, epidermal cells, seed cells, endosperm or embryonic tissue. The fine chemical produced in the process according to the invention may, however, also be isolated from the plant. In case of bis-glyceryl phosphate (polar fraction), glycerol (lipid fraction), glycerol (polar fraction), glycerol phosphate (lipid fraction), glycerol-3-phosphate (polar fraction), or triacylglycerol this can be in free form or bound to proteins. Fine chemical(s) produced by this process can be harvested by harvesting the non-human organisms either from the culture in which they grow or from the field. For example, this can be done via squeezing, grinding and/or extraction, salt precipitation and/or ion-exchange chromatography of the plant parts, preferably the plant seeds, plant fruits, plant tubers and the like.

[100978] [0437.1.7.19] to [0447.1.7.19] for the disclosure of theses paragraphs see [0437.1.7.7] to [0447.1.7.7] above

[100979] The identity and purity of the isolated compound(s) can be determined by prior art techniques. These include high performance liquid chromatography (HPLC), spectroscopic methods, mass spectrometry (MS), staining methods, thin-layer chromatography, NIRS, enzyme assay or microbiological assays. These analytical methods are summarized for example in: Patek et al., Appl. Environ. Microbiol. 60, 133 (1994), Malakhova et al., Biotekhnologiya 11, 27 (1996); Schmidt et al., Bioprocess Engineer. 19, 67 (1998), Ullmann's Encyclopedia of Industrial Chemistry (1996) Vol. A27, VCH, Weinheim, pp. 89-90, pp. 521-540, pp. 540-547, pp. 559-566, 575-581 and pp. 581-587, Michal G., “Biochemical Pathways: An Atlas of Biochemistry and Molecular Biology”, John Wiley and Sons (1999); Fallon A. et al. “Applications of HPLC in Biochemistry” in “Laboratory Techniques in Biochemistry and Molecular Biology”, Vol. 17 (1987).

[100980] The fine chemical of the invention can for example be analyzed advantageously via HPLC or GC separation methods and detected by MS or MSMS methods. The unambiguous detection for the presence of the fine chemical containing products can be obtained by analyzing recombinant organisms using analytical standard methods: GC, GC-MS or TLC, as described on several occasions by Christie and the references therein (1997, in: Advances on Lipid Methodology, Fourth Edition: Christie, Oily Press, Dundee, 119-169; 1998, GaschromatographieMassenspektrometrie-Verfahren [Gas chromatography/mass spectrometric methods], Lipide 33:343-353). The material to be analyzed can be disrupted by sonication, grinding in a glass mill, liquid nitrogen and grinding, cooking, or via other applicable methods; see also Biotechnology of Vitamins, Pigments and Growth Factors, Edited by Erik J. Vandamme, London, 1989, p.96 to 103. Glycerolipids can be separated by two-dimensional t.l.c., and the fatty acids are analysed by gas chromatography as described by Douce & Joyar (Methods Enzymol. 69, 290-301, 1980).

[100981] Glycerolipids may further be analized according to vanderest et al.(Plant Physiology, September 2002, Vol. 130, pp. 244-255).

[100982] The fine chemical obtained in the process are suitable as starting material for the synthesis of further products of value.

[100983] Accordingly, the present invention relates a method for the production of a pharmaceuticals, food stuff, animal feeds, nutrients or cosmetics comprising the steps of the process according to the invention, including the isolation of the fine chemical glycerol and/or derivates produced or a composition produced comprising the fine chemical, and, if desired, formulating the product with a pharmaceutical acceptable carrier or formulating the product in a form acceptable for an application in agriculture or formulating the product in a form acceptable for an application as food stuff, animal feed, nutrient or cosmetic. A further embodiment according to the invention is the use of the fine chemical produced in the process or of the transgenic non-human organisms in animal feeds, foodstuffs, medicines, food supplements, cosmetics or pharmaceuticals.

[100984] [0450.1.7.19] to [0452.1.7.19] for the disclosure of these paragraphs see [0450.1.7.7] to [0452.1.7.7] above

[100985] Further in another embodiment, the present invention relates to the use of the nucleic acid molecule of the invention or used in the method of the invention alone or in combination with other genes of the respective fine chemical synthesis for example of the glycerol and/or derivates and/or lipid biosynthesis, the polypeptide of the invention or used in the method of the invention, the nucleic acid construct of the invention, the expression cassette of the invention, the vector of the invention, the plant or plant tissue or the host cell of the invention, for the production of plant resistant to a herbicide inhibiting eventually the production of the fine chemical.

[100986] for the disclosure of this paragraph see [0454.1.7.7] above

[100987] In one embodiment, the present invention relates to a method for the identification of a gene product conferring the production of or an increase in the fine chemical production in a non-human cell, comprising the following steps:
[100988] (a) contacting, e.g. hybridizing, a, some or all nucleic acid molecules of a sample, e.g. cells, tissues, plants or microorganisms or a nucleic acid library, which can contain a candidate gene encoding a gene product conferring the production of or an increase in the fine chemical after expression, with the nucleic acid molecule of the present invention as shown in the respective line in column 5 or 8 of Table I A or B of application no. 19, preferably the coding region thereof, or a fragment or homolog thereof;
[100989] (b) identifying the nucleic acid molecules, which hybridize under relaxed stringent conditions with the nucleic acid molecule of the present invention in particular to the nucleic acid molecule sequence shown in the respective line in Table I, application no. 19, columns 5 or 8, preferably the coding region thereof, of a fragment or a homolog thereof, preferably in Table I B, application no. 19, columns 5 or 8, preferably the coding region thereof, of a fragment or a homolog thereof, and, optionally, isolating the full length cDNA clone or complete genomic clone;
[100990] (c) introducing the candidate nucleic acid molecules or a fragment thereof in host cells, preferably in a plant cell or a microorganism, appropriate for producing the fine chemical;
[100991] (d) expressing the identified nucleic acid molecules in the host cells for which the production of or the increased production of the fine chemical is desired;
[100992] (e) assaying the fine chemical level in the non-human host cells; and
[100993] (f) identifying the nucleic acid molecule and/or its gene product which expression confers the production of or an increase in the fine chemical level in the non-human host cell after expression compared to the wild type.

[100994] for the disclosure of this paragraph see [0456.1.7.7] above

[100995] In another embodiment, the present invention relates to a method for the identification of a gene product conferring the production of or an increase in the fine chemical production in a cell, comprising the following steps:
[100996] (a) identifying a nucleic acid molecule of a non-human organism, which is at least 20%, 25%, 30%, 35%, 40%, 50%, 60%, 70%, 80%, 90%, 95%, 96%, 97%, 98%, 99%, 99.5% or more homolog to the nucleic acid molecule encoding a polypeptide comprising the polypeptide molecule as shown in the respective line in column 5 or 8 of Table II, application no. 19, or comprising a consensus sequence or a polypeptide motif as shown in the respective line in column 8 of Table IV, application no. 19, or being encoded by a nucleic acid molecule comprising a polynucleotide as shown in the respective line in column 5 or 8 of Table I, application no. 19, preferably the coding region thereof, or a homologue thereof as described herein, for example via homology search in a data bank;
[100997] (b) introducing the candidate nucleic acid molecule in non-human host cells, preferably in a plant cell or a microorganism, appropriate for producing the fine chemical;
[100998] (c) expressing the identified nucleic acid molecule in the non-human host cells;
[100999] (d) assaying the fine chemical level in the non-human host cells; and
[101000] (e) identifying the nucleic acid molecule and its gene product which expression confers the production of or an increase in the fine chemical level in the non-human host cell after expression compared to the wild type.

[101001] for the disclosure of this paragraph see [0458.1.7.7] above

[101002] Further, the nucleic acid molecules disclosed herein, in particular the nucleic acid molecules shown in the respective line in column 5 or 8 of Table I A or B of application no. 19, may be sufficiently homologous to the sequences of related species such that these nucleic acid molecules may serve as markers for the construction of a genomic map in related organism or for association mapping. Furthermore natural variation in the genomic regions corresponding to nucleic acids disclosed herein, in particular the nucleic acid molecule shown in the respective line in column 5 or 8 of Table I A or B, of application no. 19 or homologous thereof, may lead to variation in the activity of the proteins disclosed herein, in particular the proteins comprising polypeptides as shown in the respective lines in column 5 or 8 of Table II A or B, of application no. 19 or their homologous, or comprising the consensus sequence or the polypeptide motif as shown in the respective line in column 8 of Table IV, and in consequence in a natural variation of an increased fine chemical production.

[101003] In consequence natural variation eventually also exists in form of more active allelic variants leading already to a relative increase in the fine chemical. Different variants of the nucleic acid molecules disclosed herein, in particular the nucleic acid molecules comprising the nucleic acid molecules as shown in the respective line in column 5 or 8 of Table I A or B of application no. 19, preferably the coding region thereof, which corresponds to different levels of increase in fine chemical, e.g. different levels of increase in fine chemical, can be indentified and used for marker assisted breeding for an enhanced production of the fine chemical.

[101004] Natural variation may also exist in the regulatory regions, e.g. the promoters of the natural genes comprising the nucleic acid molecules as shown in the respective line column 5 or 8 of Table I A or B of application no. 19, preferably the coding region thereof, which can similarly correspond to different levels of increase in the fine chemical, e.g. different levels of increase in the fine chemical can be identified and used for marker assisted breeding for an enhanced production of the fine chemical.

[101005] Accordingly, the present invention relates to a method for breeding plants for the production of the fine chemical, comprising
[101006] (a) selecting a first plant variety with an increased production of the fine chemical, e.g. based on an increased expression of a nucleic acid molecule of the invention as disclosed herein, in particular of a nucleic acid molecule comprising a nucleic acid molecule as shown in the respective line in column 5 or 8 of Table I A or B of application no. 19, preferably the coding region thereof, or fragments or homologs thereof, or a polypeptide comprising a polypeptide as shown in the respective line in column 5 or 8 of Table II A or B of application no. 19, or fragments or homologs thereof, or comprising a consensus sequence or a polypeptide motif as shown in the respective line in column 8 of Table IV, or a homolog thereof, as described herein;
[101007] (b) associating the level of increased production of the fine chemical, with the expression level or the genomic structure of a gene encoding said polypeptide or said nucleic acid molecule;
[101008] (c) crossing the first plant variety with a second plant variety, which significantly differs in its level of fine chemical; and
[101009] (d) identifying, which of the offspring varieties has got increased production of the fine chemical by the expression level of said polypeptide or nucleic acid molecule or the genomic structure of the gene(s) encoding said polypeptide or nucleic acid molecule of the invention.

[101010] [0462.1.7.19] to [0479.1.7.19] for the disclosure of these paragraphs see [0462.1.7.7] to [0479.1.7.7] above

[101011] In a further embodiment the nucleic acid molecule of the invention, the polypeptide of the invention, the nucleic acid construct of the invention, the expression cassette of the invention, the non-human organisms, the non-human host cell, the microorganisms, the plant, plant cell, or the part thereof, of the invention, the vector of the invention, the agonist identified with the method of the invention, the nucleic acid molecule identified with the method of the present invention, can be used for the production of the fine chemical or of the fine chemical and one or more other derivates of glycerol and/or lipids.

[101012] Accordingly, the nucleic acid molecule of the invention, or the nucleic acid molecule identified with the method of the present invention or the complement sequences thereof, the polypeptide of the invention, the nucleic acid construct of the invention, the expression cassette of the invention, the non-human organism, the non-human host cell, the microorganism, the plant, plant cell, or the part thereof of the invention, the vector of the invention, the agonist identified with the method of the invention, the antibody of the present invention, can be used for the production of or the increase in the fine chemical in a non-human organism or part thereof, e.g. in a cell.

[101013] for the disclosure of this paragraph see [0481.1.7.7] above

[101014] for the disclosure of this paragraph see [0482.1.7.7] above

[101015] #

[101016] In a further embodiment the present invention is characterized by the features as disclosed in the following items:

[101017] Item 1. A process for the production of a fine chemical selected from the group consisting of: bis-glyceryl phosphate (polar fraction), glycerol (lipid fraction), glycerol (polar fraction), glycerol phosphate (lipid fraction), glycerol-3-phosphate (polar fraction) and/or triacylglycerol
[101018] (a) increasing or generating one or more activities selected from the group consisting of (DL)-glycerol-3-phosphatase, 2-C-methyl-D-erythritol 4-phosphate cytidylyltransferase, 3-oxoacyl-[acyl-carrier-protein] synthase, 49747384_SOYBEAN-protein, ABC transporter permease protein, acetyl CoA carboxylase, acid phosphatase precursor, acid shock protein, acyl transferase, adenylate kinase, alcohol dehydrogenase, amino-acid acetyltransferase, arginine decarboxylase, argininosuccinate synthetase, aromatic acid decarboxylase, At1g23110-protein, At1g67340-protein, ATP-binding component of a transport system, ATP-dependent RNA helicase, b0050-protein, b0518-protein, b0970-protein, b1003-protein, b1234-protein, b1330-protein, b1522-protein, b1556-protein, b1583-protein, b1670-protein, b1672-protein, b1703-protein, B1795-protein, b1837-protein, b2032-protein, b2099-protein, b2139-protein, b2399-protein, b2474-protein, b2613-protein, b2673-protein, b3083-protein, b3160-protein, b3777-protein, b3791-protein, b3989-protein, b4029-protein, b4050-protein, beta-hydroxylase, biotin synthase, calcium-dependent protein kinase, carbamoylphosphate synthase subunit, carbohydrate kinase, cardiolipin synthase, CBL-interacting protein kinase, CDP-diacylglycerol-glycerol-3-phosphate 3-phosphatidyltransferase, cell division control protein, cell division protein, coproporphyrinogen III oxidase, cyclin, cyclin D , dihydrolipoamide dehydrogenase, DnaJ-like chaperone, Elongation factor Tu, enterobacterial common antigen polymerase, ethanolamine utilization protein, eukaryotic translation initiation factor 5, Fe—S subunit of oxidoreductase, FKBP-type peptidyl-prolyl cis-trans isomerase (rotamase), glutamate 5-kinase, glutaredoxin, Glycine cleavage system T aminomethyltransferase, GM02LC17485-protein, heat shock protein Hsp40, heat shock transcription factor, HesB/YadR/YfhF family protein, high-affinity branched-chain amino acid transport protein, hydrolase, imidazole glycerol phosphate synthase subunit, ion-transport protein, lipoprotein precursor, L-serine dehydratase, lysozyme, lysyl-tRNA synthetase, metal-dependent hydrolase, methylglyoxal synthase, methyltransferase, Mg-protoporphyrin IX monomethyl ester oxidative cyclase 66 kD subunit, mitochondrial processing protease, mitogen-activated protein kinase, molecular chaperone portein, monothiol glutaredoxin, murein transglycosylase, NADH dehydrogenase, NADH-quinone oxidoreductase subunit, nitrate/nitrite transport protein, oxidoreductase, phenylacetic acid degradation protein, phosphate import ATP-binding protein, phosphatidylinositol 3- and 4-kinase family protein, phosphoglyceromutase, phosphotransferase, phosphotransferase system component, photosystem I reaction centre subunit, polygalacturonase, precorrin-8X methylmutase, protein phosphatase, purine-nucleoside phosphorylase, putative transport system permease protein, putrescine transport system permease protein, pyruvate kinase, recombinase A, secindependent protein translocase, serine protease, sodium/proton antiporter, ThiF family protein, threonine dehydratase, threonine synthase, threonine/serine transporter, transcription factor, transcriptional regulator, transcriptional regulator protein, trehalose-phosphatase, TTC1386-protein, vacuolar sorting protein, YAL013W-protein, ycr102c-protein, ydr273w-protein, YFL019C-protein, YHR202W-protein, YIL083C-protein, YJL062W-A-protein, YLR099C-protein, ylr326w-protein, YPL162C.protein, and Zm_{—}4842_BE510522-protein in a non-human organism or a part thereof, preferably a microorganism, a plant cell, a plant or a part thereof, as compared to a corresponding non-transformed wild type non-human organism or a part thereof; and
[101019] (b) growing the non-human organism or a part thereof under conditions which permit the production of bis-glyceryl phosphate (polar fraction), glycerol (lipid fraction), glycerol (polar fraction), glycerol phosphate (lipid fraction), glycerol-3-phosphate (polar fraction), and/or triacylglycerol or a composition comprising bis-glyceryl phosphate (polar fraction), glycerol (lipid fraction), glycerol (polar fraction), glycerol phosphate (lipid fraction), glycerol-3-phosphate (polar fraction), and/or triacylglycerol in said non-human organism or in the culture medium surrounding said non-human organism.

[101020] Item 2. A process for the production of a fine chemical selected from the group consisting of: bis-glyceryl phosphate (polar fraction), glycerol (lipid fraction), glycerol (polar fraction), glycerol phosphate (lipid fraction), glycerol-3-phosphate (polar fraction) and/or triacylglycerol, which comprises
[101021] (A)
   [101022] (i) increasing or generating of the expression of; and/or
   [101023] (ii) increasing or generating the expression of an expression product of; and/or
   [101024] (iii) increasing or generating one or more activities of an expression product encoded by;
   [101025] at least one respective nucleic acid molecule comprising a nucleic acid molecule selected from the group consisting of:
   [101026] (a) a nucleic acid molecule encoding the polypeptide shown in column 5 or 8 of Table II, preferably Table II B, application no.19, or a homolog or a fragment thereof;
   [101027] (b) a nucleic acid molecule shown in column 5 or 8 of Table I, preferably Table I B, application no.19, or a homolog or a fragment thereof (preferably the coding region thereof);
   [101028] (c) a nucleic acid molecule, which, as a result of the degeneracy of the genetic code, can be derived from a polypeptide sequence depicted in column 5 or 8 of Table II, preferably Table II B, application no.19;
   [101029] (d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 19, or the coding region thereof;
   [101030] (e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
   [101031] (f) a nucleic acid molecule which hybridizes with a nucleic acid molecule of (a), (b), (c), (d) or (e) under stringent hybridization conditions;
   [101032] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   [101033] (h) a nucleic acid molecule encoding a polypeptide comprising the consensus sequence or one or more polypeptide motifs as shown in column 8 of Table IV, application no.19;
   [101034] (i) a nucleic acid molecule encoding a polypeptide having the activity represented by a respective protein as depicted in column 5 of Table II, application no. 19;
   [101035] (j) a nucleic acid molecule which comprises a polynucleotide, which is obtained by amplifying a cDNA library or a genomic library using the primers as depicted in column 8 of Table III, application no. 19; and
   [101036] (k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
   [101037] or a nucleic acid molecule comprising a sequence which is complementary thereto;
      in a non-human organism or a part thereof, preferably a microorganism, a plant cell, a plant or a part thereof, as compared to a corresponding non-transformed wild type non-human organism or a part thereof; and
[101038] (B) growing the non-human organism or a part thereof under conditions which permit the production of bis-glyceryl phosphate (polar fraction), glycerol (lipid fraction), glycerol (polar fraction), glycerol phosphate (lipid fraction), glycerol-3-phosphate (polar fraction) and/or triacylglycerol or a composition comprising bis-glyceryl phosphate (polar fraction), glycerol (lipid fraction), glycerol (polar fraction), glycerol phosphate (lipid fraction), glycerol-3-phosphate (polar fraction) and/or triacylglycerol in said non-human organism or in the culture medium surrounding said non-human organism.

[101039] Item 3. A process of items 1 or 2, comprising of recovering bis-glyceryl phosphate (polar fraction), glycerol (lipid fraction), glycerol (polar fraction), glycerol phosphate (lipid fraction), glycerol-3-phosphate (polar fraction) and/or triacylglycerol in its free or bound form.

[101040] Item 4. The process of any one of items 1 to 3, comprising the following steps:
[101041] (a) selecting a non-human organism or a part thereof expressing a polypeptide encoded by the nucleic acid molecule characterized in item 2;
[101042] (b) mutagenizing the selected non-human organism or the part thereof;
[101043] (c) comparing the activity or the expression level of said polypeptide in the mutagenized non-human organism or the part thereof with the activity or the expression of said polypeptide of the selected non-human organisms or the part thereof;
[101044] (d) selecting the mutated non-human organisms or parts thereof, which comprise an increased activity or expression level of said polypeptide compared to the selected non-human organism or the part thereof;
[101045] (e) optionally, growing and cultivating the non-human organisms or the parts thereof; and
[101046] (f) recovering, and optionally isolating, the free or bound bis-glyceryl phosphate (polar fraction), glycerol (lipid fraction), glycerol (polar fraction), glycerol phosphate (lipid fraction), glycerol-3-phosphate (polar fraction) and/or triacylglycerol produced by the selected mutated non-human organisms or parts thereof.

[101047] Item 5. The process of any one of items 1 to 4 wherein the activity of said protein or the expression of said nucleic acid molecule is increased or generated transiently or stably.

[101048] Item 6. An isolated nucleic acid molecule comprising a nucleic acid molecule selected from the group consisting of:
[101049] (a) a nucleic acid molecule encoding a polypeptide shown in Table II, application no. 19, column 5 or 8, preferably shown in Table II B, application no. 19, column 8, or a homolog or a fragment thereof;
[101050] (b) a nucleic acid molecule shown in Table I, application no. 19, column 5 or 8, preferably shown in Table I B, application no. 19, column 8, preferably the coding region thereof, or a homolog or a fragment thereof;
[101051] (c) a nucleic acid molecule, which, as a result of the degeneracy of the genetic code, can be derived from a polypeptide sequence depicted in column 5 or 8 of Table II, application no. 19, preferably in column 8 of Table II B, application no. 19;
[101052] (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 19, preferably shown in column 8 of Table I B, application no. 19, preferably the coding region thereof;
[101053] (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
[101054] (f) a nucleic acid molecule which hybridizes with a nucleic acid molecule of (a), (b), (c), (d) or (e) under stringent hybridisation conditions;
[101055] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
[101056] (h) a nucleic acid molecule encoding a polypeptide comprising the consensus sequence or one or more polypeptide motifs as shown in column 8 of Table IV, application no. 19;
[101057] (i) a nucleic acid molecule encoding a polypeptide having the activity represented by a respective protein as depicted in column 5 of Table II, application no. 19;
[101058] (j) a nucleic acid molecule comprising a polynucleotide which is obtained by amplifying a cDNA library or a genomic library using the primers in Table III, application no. 19, column 8;
[101059] and
[101060] (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (i);
[101061] or a nucleic acid molecule comprising a sequence which is complementary thereto.

[101062] Item 7. A nucleic acid construct which confers the expression of the nucleic acid molecule as defined in item 6, comprising one or more regulatory elements.

[101063] Item 8. An expression cassette comprising
[101064] (a) a promoter, preferably selected from the group consisting of Big35S, PCUbi, Super and USP;
[101065] (b) a nucleic acid molecule as defined in item 6.

[101066] Item 9. A vector comprising the nucleic acid molecule as defined in item 6 or the nucleic acid construct as claimed in item 7 or the expression cassette as claimed in item 8.

[101067] Item 10. A host cell or a plant organelle, which has been transformed with the vector as claimed in item 9 or the nucleic acid molecule as defined in item 6 or the nucleic acid construct as claimed in item 7 or the expression cassette as claimed in item 8.

[101068] Item 11. The host cell of item 10, which is a plant cell or a microorganism.

[101069] Item 12. A process for producing a polypeptide, wherein the polypeptide is expressed in a host cell as claimed in item 10 or 11.

[101070] Item 13. A polypeptide produced by the process as claimed in item 12 or encoded by the nucleic acid molecule as defined in item 6.

[101071] Item 14. An antibody, which binds specifically to the polypeptide as claimed in item 13.

[101072] Item 15. A plant tissue, propagation material, harvested material or a plant comprising the host cell as claimed in item 10 or 11.

[101073] Item 16. A process for the identification of a compound conferring an increase in bis-glyceryl phosphate (polar fraction), glycerol (lipid fraction), glycerol (polar fraction), glycerol phosphate (lipid fraction), glycerol-3-phosphate (polar fraction) and/or triacylglycerol production in a non-human organism, comprising the steps:
[101074] (a) culturing a plant cell or tissue or microorganism or maintaining a plant expressing the polypeptide encoded by the nucleic acid molecule as defined in item 6 conferring an increase in the amount of bis-glyceryl phosphate (polar fraction), glycerol (lipid fraction), glycerol (polar fraction), glycerol phosphate (lipid fraction), glycerol-3-phosphate (polar fraction) and/or triacylglycerol in a non-human organism or a part thereof and a readout system capable of interacting with the polypeptide under suitable conditions which permit the interaction of the polypeptide with said readout system in the presence of a compound or a sample comprising a plurality of compounds and capable of providing a detectable signal in response to the binding of a compound to said polypeptide under conditions which permit the expression of said readout system and of the polypeptide encoded by the nucleic acid molecule of item 6 conferring an increase in the amount of bis-glyceryl phosphate (polar fraction), glycerol (lipid fraction), glycerol (polar fraction), glycerol phosphate (lipid fraction), glycerol-3-phosphate (polar fraction) and/or triacylglycerols in a non-human organism or a part thereof;
[101075] (b) identifying if the compound is an effective agonist by detecting the presence or absence or increase of a signal produced by said readout system.

[101076] Item 17. A method for the production of an agricultural composition comprising the steps of the method of item 16 and formulating the compound identified in item 16 in a form acceptable for an application in agriculture.

[101077] Item 18. A composition comprising the nucleic acid molecule as defined in item 6, the polypeptide as claimed in item 13, the nucleic acid construct as claimed in item 7, the expression cassette as claimed in item 8, the vector as claimed in item 9, the compound as claimed in item 16, the antibody as claimed in item 14, and optionally an agricultural acceptable carrier.

[101078] Item 19. Use of the nucleic acid molecule as defined in item 6 for the identification of a nucleic acid molecule conferring an increase in bis-glyceryl phosphate (polar fraction), glycerol (lipid fraction), glycerol (polar fraction), glycerol phosphate (lipid fraction), glycerol-3-phosphate (polar fraction) and/or triacylglycerol after expression.

[101079] Item 20. Cosmetic, pharmaceutical, food or feed composition comprising the nucleic acid moleculeas defined in item 6, the polypeptide as claimed in item 13, the nucleic acid construct as claimed in item 7, the expression cassette as claimed in item 8, the vector as claimed in item 9, the antibody as claimed in item 14 , the plant or plant tissue as claimed in item 15, the harvested material as claimed in item 15 or the host cell of item as claimed in item s 10 or 11.

[101080] Item 21. Use of the nuclei acid molecule as defined in item 6, the polypeptide as claimed in item 13, the nucleic acid construct as claimed in item 7, the expression cassette as claimed in item 8, the vector as claimed in item 9, the plant or plant tissue as claimed in item 15, or the host cell of item 10 to 11 for the production of plant resistant to a herbicide inhibiting the production of bis-glyceryl phosphate (polar fraction), glycerol (lipid fraction), glycerol (polar fraction), glycerol phosphate (lipid fraction), glycerol-3-phosphate (polar fraction) and/or triacylglycerol.

[101081] [0483.1.7.19] to [0492.1.7.19] for the disclosure of these paragraphs see [0483.1.7.7] to [0492.1.7.7] above

### Example 10

### Purification of a Fine Chemical Glycerol and/or Derivatel e.g. Bis-Glyceryl Phosphate (Polar Fraction), Glycerol (Lipid Fraction), Glycerol (Polar Fraction), Glycerol Phosphate (Lipid Fraction), Glycerol-3-Phosphate (Polar Fraction) and/or Triacylglycerol

[101082] The fine chemical can be recovered from cells or from the supernatant of the above-described culture by a variety of methods known in the art. For example, the culture supernatant is recovered first. To this end, the cells are harvested from the culture by slow centrifugation. Cells can generally be disrupted or lysed by standard techniques such as mechanical force or sonication. The cell debris is removed by centrifugation and the supernatant fraction, if appropriate together with the culture supernatant, is used for the further purification of the polyalcohols. However, it is also possible to process the supernatant alone if the polyalcohols are present in the supernatant in sufficiently high a concentration. In this case, the fine chemical or mixture thereof, can be purified further for example via extraction and/or salt precipitation or via ion-exchange chromatography or as described above.

[101083] Identity and purity of the fine chemical isolated can be determined by standard techniques of the art. They encompass old techniques such as photometric methods and new techniques such as high-performance liquid chromatography (HPLC), gas chromatography (GC), spectroscopic methods, mass spectrometry (MS), staining methods, thin-layer chromatography, NIRS, enzyme assay or microbiological assays.

[101084] [0494.1.7.19] for the disclosure of this paragraph see [0494.1.7.7] above

### Example 11a

### PCR Amplification of the Sequences

[101085] Unless otherwise specified, standard methods as described in Sambrook et al., Molecular Cloning: A laboratory manual, Cold Spring Harbor 1989, Cold Spring Harbor Laboratory Press are used.

[101086] The inventive sequences as shown in the respective line in Table I, column 5, preferably the coding region thereof, were amplified by PCR as described in the protocol of the Pfu Ultra, Pfu Turbo or Herculase DNA polymerase (Stratagene). The composition for the protocol of the Pfu Ultra, Pfu Turbo or Herculase DNA polymerase was as follows: 1×PCR buffer (Stratagene), 0.2 mM of each dNTP, 100 ng genomic DNA of Saccharomyces cerevisiae (strain S288C; Research Genetics, Inc., now Invitrogen), Escherichia coli (strain MG1655; E. coli Genetic Stock Center), Synechocystis sp. (strain PCC6803), Azotobacter vinelandii (strain N.R. Smith,16), Thermus thermophilus (HB8) or 50 ng cDNA from various tissues and development stages of Arabidopsis thaliana (ecotype Columbia), Physcomitrella patens, Glycine max (variety Resnick), Brassica napus, Oryza sativa or Zea mays (variety B73, Mo17, A188), 50 pmol forward primer, 50 pmol reverse primer, with or without 1 M Betaine, 2.5 u Pfu Ultra, Pfu Turbo or Herculase DNA polymerase.

[101087] The amplification cycles were as follows:

[101088] 1 cycle of 2-3 minutes at 94-95° C., then 25-36 cycles with 30-60 seconds at 94-95° C., 30-45 seconds at 50-60° C. and 210-480 seconds at 72° C., followed by 1 cycle of 5-10 minutes at 72° C., then 4-16° C.—preferably for Saccharomyces cerevisiae, Escherichia coli, Synechocystis sp., Azotobacter vinelandii, Thermus thermophilus.

[101089] In case of Arabidopsis thaliana, Brassica napus, Glycine max, Oryza sativa, Physcomitrella patens, Zea mays the amplification cycles were as follows:

[101090] 1 cycle with 30 seconds at 94° C., 30 seconds at 61° C., 15 minutes at 72° C., then 2 cycles with 30 seconds at 94° C., 30 seconds at 60° C., 15 minutes at 72° C., then 3 cycles with 30 seconds at 94° C., 30 seconds at 59° C., 15 minutes at 72° C., then 4 cycles with 30 seconds at 94° C., 30 seconds at 58° C., 15 minutes at 72° C., then 25 cycles with 30 seconds at 94° C., 30 seconds at 57° C., 15 minutes at 72° C., then 1 cycle with 10 minutes at 72° C., then finally 4-16° C.

[101091] RNAs were generated with the RNeasy Plant Kit according to the standard protocol (Qiagen) and Superscript II Reverse Transkriptase was used to produce double stranded cDNA according to the standard protocol (Invitrogen).

[101092] ORF specific primer pairs for the genes to be expressed are shown in the respective line in Table III, column 8. The following adapter sequences were added to Saccharomyces cerevisiae ORF specific primers (see Table III) for cloning purposes:

SEQ ID NO: 20

| | | |
|---|---|---|
| i) | foward primer: | 5′-GGAATTCCAGCTGACCACC-3′ |

SEQ ID NO: 21

| | | |
|---|---|---|
| ii) | reverse primer: | 5′-GATCCCCGGGAATTGCCATG-3′ |

[101093] These adaptor sequences allow cloning of the ORF into the various vectors containing the Resgen adaptors, see table column E of Table c.

[101094] The following adapter sequences were added to Saccharomyces cerevisiae, Escherichia coli, Synechocystis sp., Azotobacter vinelandii, Thermus thermophilus, Arabidopsis thaliana, Brassica napus, Glycine max, Oryza sativa , Physcomitrella patens, or Zea mays ORF specific primers for cloning purposes:

| SEQ ID NO: 22 | | |
|---|---|---|
| iii) | forward primer: | 5′-TTGCTCTTCC-3′ |
| SEQ ID NO: 23 | | |
| iiii) | reverse primer: | 5′-TTGCTCTTCG-3′ |

[101095] The adaptor sequences allow cloning of the ORF into the various vectors containing the Colic adaptors, see table column E of Table c.

[101096] Therefore for amplification and cloning of Saccharomyces cerevisiae SEQ ID NO: 14769, a primer consisting of the adaptor sequence i) and the ORF specific sequence SEQ ID NO: 14815 and a second primer consisting of the adaptor sequence ii) and the ORF specific sequence SEQ ID NO: 14816 were used.

[101097] For amplification and cloning of Escherichia coli SEQ ID NO: 7081, a primer consisting of the adaptor sequence iii) and the ORF specific sequence SEQ ID NO: 7261 and a second primer consisting of the adaptor sequence iiii) and the ORF specific sequence SEQ ID NO: 7262 were used.

[101098] For amplification and cloning of Synechocystis sp. SEQ ID NO: 11471, a primer consisting of the adaptor sequence iii) and the ORF specific sequence SEQ ID NO: 11983 and a second primer consisting of the adaptor sequence iiii) and the ORF specific sequence SEQ ID NO: 11984 were used.

[101099] For amplification and cloning of Arabidopsis thaliana SEQ ID NO: 1061, a primer consisting of the adaptor sequence iii) and the ORF specific sequence SEQ ID NO: 1293 and a second primer consisting of the adaptor sequence iiii) and the ORF specific sequence SEQ ID NO: 1294 were used.

[101100] For amplification and cloning of Azotobacter vinelandii SEQ ID NO: 6040, a primer consisting of the adaptor sequence iii) and the ORF specific sequence SEQ ID NO: 6070 and a second primer consisting of the adaptor sequence iiii) and the ORF specific sequence SEQ ID NO: 6071 were used.

[101101] For amplification and cloning of Glycine max SEQ ID NO: 69, a primer consisting of the adaptor sequence iii) and the ORF specific sequence SEQ ID NO: 343 and a second primer consisting of the adaptor sequence iiii) and the ORF specific sequence SEQ ID NO: 344 were used.

[101102] For amplification and cloning of Thermus thermophilus SEQ ID NO: 60301, a primer consisting of the adaptor sequence iii) and the ORF specific sequence SEQ ID NO: 60849 and a second primer consisting of the adaptor sequence iiii) and the ORF specific sequence SEQ ID NO: 60850 were used.

[101103] For amplification and cloning of Zea mays SEQ ID NO: 68413, a primer consisting of the adapfor sequence iii) and the ORF specific sequence SEQ ID NO: 68531 and a second primer consisting of the adaptor sequence iiii) and the ORF specific sequence SEQ ID NO: 68532 were used.

[101104] Following these examples every sequence disclosed in Table I, preferably column 5, especially the coding region thereof can be cloned by fusing the adaptor sequences to the respective specific primers sequences as disclosed in Table III, column 8 using the respective vectors shown in Table c.

[101105] [0496.1.7.19] to [0499.1.7.19] for the disclosure of these paragraphs see [0496.1.7.7] to [0499.1.7.7] above

### Example 11e

### Cloning of Inventive Sequences as shown in Table I, Column 5 in the Different Expression Vectors

[101106] For cloning of for example SEQ ID NO: 7081 from Escherichia coli or any other ORF from Escherichia coli, Synechocystis sp., Azotobacter vinelandii, Thermus thermophilus, Arabidopsis thaliana, Glycine max, or Zea mays the vector DNA was treated with the restriction enzymes Pacl and Ncol following the standard protocol (MBI Fermentas).

[101107] For cloning for example the ORFs of SEQ ID NO: 14769 from Saccharomyces cerevisiae or any other ORF from Saccharomyces cerevisiae into vectors containing the Resgen adaptor sequence the respective vector DNA was treated with the restriction enzyme Ncol.

[101108] For cloning of for example ORFs SEQ ID NO: 66274 from Saccharomyces cerevisiae or any other ORF from Saccharomyces cerevisiae into vectors containing the Colic adaptor sequence, the respective vector DNA was treated with the restriction enzymes Pacl and Ncol following the standard protocol (MBI Fermentas).

[101109] In all cases the reaction was stopped by inactivation at 70° C. for 20 minutes and purified over QlAquick or NucleoSpin Extract II columns following the standard protocol (Qiagen or Macherey-Nagel). Then the PCR-product representing the amplified ORF with the respective adapter sequences and the vector DNA were treated with T4 DNA polymerase according to the standard protocol (MBI Fermentas) to produce single stranded overhangs with the parameters 1 unit T4 DNA polymerase at 37° C. for 2-10 minutes for the vector and 1-2 u T4 DNA polymerase at 15-17° C. for 10-60 minutes for the PCR product representing SEQ ID NO: 7081.

[101110] The reaction was stopped by addition of high-salt buffer and purified over QlAquick or NucleoSpin Extract II columns following the standard protocol (Qiagen or Macherey-Nagel).

[101111] According to this example the skilled person is able to clone all sequences disclosed in Table I, preferably column 5 or column 8, especially the coding region thereof. Approximately 30-60 ng of prepared vector and a defined amount of prepared amplificate were mixed and hybridized at 65° C. for 15 minutes followed by 37° C. 0.1° C/1 seconds, followed by 37° C. 10 minutes, followed by 0.1° C/1 seconds, then 4-10 ° C.

[101112] The ligated constructs were transformed in the same reaction vessel by addition of competent E. coli cells (strain DH5alpha) and incubation for 20 minutes at 1° C. followed by a heat shock for 90 seconds at 42° C. and cooling to 1-4° C. Then, complete medium (SOC) was added and the mixture was incubated for 45 minutes at 37° C. The entire mixture was subsequently plated onto an agar plate with 0.05 mg/ml kanamycin and incubated overnight at 37° C.

[101113] The outcome of the cloning step was verified by amplification with the aid of primers which bind upstream and downstream of the integration site, thus allowing the amplification of the insertion. The amplifications were carried out as described in the protocol of Taq DNA polymerase (Gibco-BRL).

[101114] The amplification cycles were as follows: 1 cycle of 1-5 minutes at 94° C., followed by 35 cycles of in each case 15-60 seconds at 94° C., 15-60 seconds at 50-66° C. and 5-15 minutes at 72° C., followed by 1 cycle of 10 minutes at 72° C., then 4-16° C.

[101115] Several colonies were checked, but only one colony for which a PCR product of the expected size was detected was used in the following steps.

[101116] A portion of this positive colony was transferred into a reaction vessel filled with complete medium (LB) supplemented with kanamycin and incubated overnight at 37° C.

[101117] The plasmid preparation was carried out as specified in the Qiaprep or NucleoSpin Multi-96 Plus standard protocol (Qiagen or Macherey-Nagel).

[101118] [0501.1.7.19] to [0503.1.7.19] for the disclosure of these paragraphs see [0501.1.7.7] to [0503.1.7.7] above

[101119] Table d showing results of plant analyses

TABLE d

| SeqID | Target | Locus | Metabolite | Source | Promotor | Method | Min | Max |
|---|---|---|---|---|---|---|---|---|
| 17451 | non-targ | At1g07430 | glycerol phosphate | ARA_LEAF | p-PcUBI | GC | 21 | 63 |
| | | | (lf) | | | | | |
| 102086 | non-targ | At1g23110 | bis-glyceryl phos- | ARA_LEAF | p-PcUBI | GC | 264 | 530 |
| | | | phate (pf) | | | | | |
| 17968 | non-targ | At1g36730 | bis-glyceryl phos- | ARA_LEAF | p-PcUBI | GC | 38 | 72 |
| | | | phate (pf) | | | | | |
| 17968 | non-targ | At1g36730 | glycerol phosphate | ARA_LEAF | p-PcUBI | GC | 24 | 39 |
| | | | (lf) | | | | | |
| 19364 | non-targ | At1g67340 | triacylglycerol | ARA_LEAF | p-PcUBI | LC | 39 | 135 |
| 1061 | non-targ | At1g68320 | glycerol (If) | ARA_LEAF | p-PcUBI | GC | 16 | 57 |
| 19419 | non-targ | At1g72770 | bis-glyceryl phos- | ARA_LEAF | p-PcUBI | GC | 40 | 78 |
| | | | phate (pf) | | | | | |
| 19419 | non-targ | At1g72770 | glycerol (lf) | ARA_LEAF | p-PcUBI | GC | 23 | 67 |
| 19419 | non-targ | At1g72770 | glycerol phosphate | ARA_LEAF | p-PcUBI | GC | 17 | 56 |
| | | | (lf) | | | | | |
| 19919 | non-targ | At2g30360 | bis-glyceryl phos- | ARA_LEAF | p-PcUBI | GC | 39 | 69 |
| | | | phate (pf) | | | | | |
| 21106 | non-targ | At2g46500 | bis-glyceryl phos- | ARA_LEAF | p-PcUBI | GC | 45 | 68 |
| | | | phate (pf) | | | | | |
| 68657 | non-targ | At3g02990 | triacylglycerol | ARA_LEAF | p-PcUBI | LC | 39 | 91 |
| 109717 | non-targ | At4g09960 | glycerol (lf) | ARA_LEAF | p-PcUBI | GC | 18 | 25 |
| 23482 | non-targ | At4g15660 | glycerol (lf) | ARA_LEAF | p-PcUBI | GC | 16 | 85 |
| 23482 | non-targ | At4g15660 | triacylglycerol | ARA_LEAF | p-PcUBI | LC | 33 | 158 |
| 3279 | non-targ | At4g15670 | glycerol (lf) | ARA_LEAF | p-PcUBI | GC | 53 | 59 |
| 3279 | non-targ | At4g15670 | triacylglycerol | ARA_LEAF | p-PcUBI | LC | 38 | 150 |
| 68849 | non-targ | At4g26080 | glycerol phosphate | ARA_LEAF | p-PcUBI | GC | 17 | 35 |
| | | | (lf) | | | | | |
| 24311 | non-targ | At4g34160 | bis-glyceryl phos- | ARA_LEAF | p-PcUBI | GC | 50 | 76 |
| | | | phate (pf) | | | | | |
| 4348 | non-targ | At4g35310 | bis-glyceryl phos- | ARA_LEAF | p-PcUBI | GC | 39 | 145 |
| | | | phate (pf) | | | | | |
| 24438 | non-targ | At5g03720 | glycerol (lf) | ARA_LEAF | p-PcUBI | GC | 20 | 29 |
| 24438 | non-targ | At5g03720 | triacylglycerol | ARA_LEAF | p-PcUBI | LC | 37 | 99 |
| 4904 | non-targ | At5g18600 | glycerol (lf) | ARA_LEAF | p-PcUBI | GC | 17 | 34 |
| 5318 | non-targ | At5g57050 | glycerol phosphate | ARA_LEAF | p-PcUBI | GC | 19 | 35 |
| | | | (lf) | | | | | |
| 25498 | non-targ | At5g59220 | glycerol phosphate | ARA_LEAF | p-PcUBI | GC | 18 | 39 |
| | | | (lf) | | | | | |
| 70038 | plastidic | At5g63680 | glycerol phosphate | ARA_LEAF | p-PcUBI | GC | 19 | 47 |
| | | | (lf) | | | | | |
| 70715 | non-targ | AVIN- | triacylglycerol | ARA_LEAF | p-PcUBI | LC | 35 | 52 |
| | | DRAFT_0539 | | | | | | |
| 26196 | non-targ | Avin- | glycerol phosphate | ARA_LEAF | p-PcUBI | GC | 19 | 29 |
| | | DRAFT_1624 | (lf) | | | | | |
| 114083 | non-targ | Avin- | glycerol (lf) | ARA_LEAF | p-PcUBI | GC | 19 | 29 |
| | | DRAFT_1788 | | | | | | |
| 114198 | non-targ | Avin- | bis-glyceryl phos- | ARA_LEAF | p-PcUBI | GC | 38 | 64 |
| | | DRAFT_2010 | phate (pf) | | | | | |
| 6040 | non-targ | Avin- | bis-glyceryl phos- | ARA_LEAF | p-PcUBI | GC | 39 | 71 |
| | | DRAFT_2091 | phate (pf) | | | | | |
| 6040 | non-targ | Avin- | triacylglycerol | ARA_LEAF | p-PcUBI | LC | 31 | 40 |
| | | DRAFT_2091 | | | | | | |
| 29500 | non-targ | Avin- | bis-glyceryl phos- | ARA_LEAF | p-PcUBI | GC | 38 | 80 |
| | | DRAFT_3253 | phate (pf) | | | | | |
| 111155 | non-targ | Avin- | glycerol phosphate | ARA_LEAF | p-PcUBI | GC | 17 | 56 |
| | | DRAFT_3577 | (lf) | | | | | |
| 114231 | non-targ | Avin- | glycerol (lf) | ARA_LEAF | p-PcUBI | GC | 34 | 60 |
| | | DRAFT_3629 | | | | | | |
| 73719 | non-targ | Avin- | bis-glyceryl phos- | ARA_LEAF | p-PcUBI | GC | 39 | 53 |
| | | DRAFT_4606 | phate (pf) | | | | | |
| 6510 | non-targ | Avin- | triacylglycerol | ARA_LEAF | p-PcUBI | LC | 57 | 69 |
| | | DRAFT_5103 | | | | | | |
| 32648 | non-targ | Avin- | glycerol phosphate | ARA_LEAF | p-PcUBI | GC | 19 | 65 |
| | | DRAFT_5467 | (lf) | | | | | |
| 103433 | non-targ | AY623894 | bis-glyceryl phos- | ARA_LEAF | p-PcUBI | GC | 37 | 93 |
| | | | phate (pf) | | | | | |
| 34889 | plastidic | B0004 | triacylglycerol | ARA_LEAF | p-Super | LC | 56 | 127 |
| 103958 | non-targ | B0050 | glycerol phosphate | ARA_LEAF | p-Super | GC | 18 | 39 |
| | | | (lf) | | | | | |
| 7081 | non-targ | B0161 | bis-glyceryl phos- | ARA_LEAF | p-Super | GC | 38 | 78 |
| | | | phate (pf) | | | | | |
| 7081 | non-targ | B0161 | triacylglycerol | ARA_LEAF | p-Super | LC | 47 | 112 |
| 7333 | non-targ | B0449 | bis-glyceryl phos- | ARA_LEAF | p-Super | GC | 48 | 187 |
| | | | phate (pf) | | | | | |
| 35936 | non-targ | B0518 | glycerol (lf) | ARA_LEAF | p-Super | GC | 25 | 54 |
| 152576 | plastidic | B0755 | bis-glyceryl phos- | ARA_LEAF | p-Super | GC | 51 | 83 |
| | | | phate (pf) | | | | | |
| 131359 | plastidic | B0856 | glycerol (pf) | ARA_LEAF | p-Super | GC | 39 | 57 |
| 36670 | non-targ | B0963 | glycerol (lf) | ARA_LEAF | p-Super | GC | 16 | 23 |
| 36670 | non-targ | B0963 | glycerol (pf) | ARA_LEAF | p-Super | GC | 46 | 105 |
| 144479 | non-targ | B0970 | bis-glyceryl phos- | ARA_LEAF | p-Super | GC | 60 | 205 |
| | | | phate (pf) | | | | | |
| 7941 | non-targ | B1003 | bis-glyceryl phos- | ARA_LEAF | p-Super | GC | 52 | 148 |
| | | | phate (pf) | | | | | |
| 114552 | plastidic | B1095 | bis-glyceryl phos- | ARA_LEAF | p-Super | GC | 53 | 164 |
| | | | phate (pf) | | | | | |
| 37400 | non-targ | B1186 | glycerol (lf) | ARA_LEAF | p-Super | GC | 17 | 80 |
| 112111 | non-targ | B1234 | triacylglycerol | ARA_LEAF | p-Super | LC | 41 | 80 |
| 98778 | plastidic | B1249 | glycerol (lf) | ARA_SEED_2 | p-USP | GC | 10 | 22 |
| 38226 | non-targ | B1330 | glycerol phosphate | ARA_LEAF | p-Super | GC | 25 | 55 |
| | | | (lf) | | | | | |
| 76890 | non-targ | B1343 | bis-glyceryl phos- | ARA_LEAF | p-Super | GC | 38 | 51 |
| | | | phate (pf) | | | | | |
| 131489 | non-targ | B1391 | bis-glyceryl phos- | ARA_LEAF | p-Super | GC | 44 | 74 |
| | | | phate (pf) | | | | | |
| 38266 | plastidic | B1431 | bis-glyceryl phos- | ARA_LEAF | p-Super | GC | 45 | 94 |
| | | | phate (pf) | | | | | |
| 7947 | non-targ | B1522 | bis-glyceryl phos- | ARA_LEAF | p-Super | GC | 64 | 135 |
| | | | phate (pf) | | | | | |
| 152991 | non-targ | B1554 | bis-glyceryl phos- | ARA_LEAF | p-Super | GC | 39 | 74 |
| | | | phate (pf) | | | | | |
| 145142 | plastidic | B1556 | bis-glyceryl phos- | ARA_LEAF | p-Super | GC | 44 | 119 |
| | | | phate (pf) | | | | | |
| 78138 | non-targ | B1583 | triacylglycerol | ARA_LEAF | p-Super | LC | 30 | 32 |
| 78154 | non-targ | B1589 | bis-glyceryl phos- | ARA_LEAF | p-Super | GC | 41 | 94 |
| | | | phate (pf) | | | | | |
| 38300 | non-targ | B1597 | glycerol (lf) | ARA_LEAF | p-Super | GC | 21 | 123 |
| 78753 | non-targ | B1670 | bis-glyceryl phos- | ARA_LEAF | p-Super | GC | 53 | 94 |
| | | | phate (pf) | | | | | |
| 78771 | non-targ | B1672 | bis-glyceryl phos- | ARA_LEAF | p-Super | GC | 39 | 72 |
| | | | phate (pf) | | | | | |
| 124907 | non-targ | B1703 | triacylglycerol | ARA_LEAF | p-Super | LC | 35 | 49 |
| 153072 | non-targ | B1736 | bis-glyceryl phos- | ARA_LEAF | p-Super | GC | 38 | 85 |
| | | | phate (pf) | | | | | |
| 131539 | non-targ | B1795 | triacylglycerol | ARA_LEAF | p-Super | LC | 40 | 82 |
| 8033 | plastidic | B1814 | glycerol-3- | ARA_SEED_2 | p-USP | LC | 54 | 199 |
| | | | phosphate (pf) | | | | | |
| 78953 | non-targ | B1837 | glycerol phosphate | ARA_LEAF | p-Super | GC | 20 | 38 |
| | | | (lf) | | | | | |
| 79056 | non-targ | B1897 | triacylglycerol | ARA_LEAF | p-Super | LC | 41 | 60 |
| 99371 | non-targ | B2023 | glycerol (lf) | ARA_SEED_2 | p-USP | GC | 13 | 19 |
| 8920 | non-targ | B2032 | glycerol phosphate | ARA_LEAF | p-Super | GC | 22 | 67 |
| | | | (lf) | | | | | |
| 115595 | non-targ | B2099 | glycerol (lf) | ARA_LEAF | p-Super | GC | 16 | 25 |
| 125085 | plastidic | B2139 | bis-glyceryl phos- | ARA_LEAF | p-Super | GC | 42 | 43 |
| | | | phate (pf) | | | | | |
| 39040 | non-targ | B2178 | glycerol (lf) | ARA_LEAF | p-Super | GC | 17 | 44 |
| 146238 | non-targ | B2389 | glycerol phosphate | ARA_LEAF | p-Super | GC | 21 | 34 |
| | | | (lf) | | | | | |
| 39237 | non-targ | B2399 | bis-glyceryl phos- | ARA_LEAF | p-Super | GC | 53 | 471 |
| | | | phate (pf) | | | | | |
| 39237 | non-targ | B2399 | glycerol (lf) | ARA_LEAF | p-Super | GC | 20 | 45 |
| 115628 | non-targ | B2439 | glycerol (lf) | ARA_LEAF | p-Super | GC | 17 | 35 |
| 115628 | non-targ | B2439 | glycerol phosphate | ARA_LEAF | p-Super | GC | 21 | 31 |
| | | | (lf) | | | | | |
| 40299 | non-targ | B2461 | glycerol (lf) | ARA_LEAF | p-Super | GC | 20 | 42 |
| 40329 | non-targ | B2474 | bis-glyceryl phos- | ARA_LEAF | p-Super | GC | 38 | 83 |
| | | | phate (pf) | | | | | |
| 40329 | non-targ | B2474 | glycerol (lf) | ARA_LEAF | p-Super | GC | 16 | 28 |
| 99898 | non-targ | B2546 | glycerol (lf) | ARA_LEAF | p-Super | GC | 22 | 24 |
| 99898 | non-targ | B2546 | glycerol phosphate | ARA_LEAF | p-Super | GC | 18 | 52 |
| | | | (lf) | | | | | |
| 40665 | non-targ | B2613 | bis-glyceryl phos- | ARA_LEAF | p-Super | GC | 42 | 82 |
| | | | phate (pf) | | | | | |
| 40726 | non-targ | B2634 | glycerol (pf) | ARA_LEAF | p-Super | GC | 58 | 125 |
| 9244 | non-targ | B2673 | glycerol (lf) | ARA_LEAF | p-Super | GC | 17 | 20 |
| 9244 | non-targ | B2673 | glycerol phosphate | ARA_LEAF | p-Super | GC | 19 | 47 |
| | | | (lf) | | | | | |
| 80756 | non-targ | B2701 | bis-glyceryl phos- | ARA_LEAF | p-Super | GC | 55 | 98 |
| | | | phate (pf) | | | | | |
| 40741 | non-targ | B2714 | triacylglycerol | ARA_LEAF | p-Super | LC | 38 | 56 |
| 81075 | non-targ | B2747 | glycerol (lf) | ARA_LEAF | p-Super | GC | 18 | 58 |
| 81576 | non-targ | B2818 | triacylglycerol | ARA_LEAF | p-Super | LC | 30 | 47 |
| 82424 | non-targ | B3083 | bis-glyceryl phos- | ARA_LEAF | p-Super | GC | 43 | 60 |
| | | | phate (pf) | | | | | |
| 153246 | non-targ | B3116 | bis-glyceryl phos- | ARA_LEAF | p-Super | GC | 38 | 121 |
| | | | phate (pf) | | | | | |
| 153395 | non-targ | B3160 | bis-glyceryl phos- | ARA_LEAF | p-Super | GC | 47 | 100 |
| | | | phate (pf) | | | | | |
| 153618 | non-targ | B3172 | bis-glyceryl phos- | ARA_LEAF | p-Super | GC | 47 | 115 |
| | | | phate (pf) | | | | | |
| 9492 | non-targ | B3256 | glycerol-3- | ARA_SEED_2 | p-USP | LC | 72 | 110 |
| | | | phosphate (pf) | | | | | |
| 42502 | non-targ | B3262 | bis-glyceryl phos- | ARA_LEAF | p-Super | GC | 59 | 124 |
| | | | phate (pf) | | | | | |
| 42502 | non-targ | B3262 | glycerol (lf) | ARA_LEAF | p-Super | GC | 16 | 60 |
| 153813 | non-targ | B3347 | bis-glyceryl phos- | ARA_LEAF | p-Super | GC | 60 | 198 |
| | | | phate (pf) | | | | | |
| 146472 | non-targ | B3457 | bis-glyceryl phos- | ARA_LEAF | p-Super | GC | 37 | 71 |
| | | | phate (pf) | | | | | |
| 136098 | non-targ | B3777 | bis-glyceryl phos- | ARA_LEAF | p-Super | GC | 39 | 85 |
| | | | phate (pf) | | | | | |
| 83726 | non-targ | B3791 | bis-glyceryl phos- | ARA_LEAF | p-Super | GC | 40 | 93 |
| | | | phate (pf) | | | | | |
| 43800 | non-targ | B3793 | bis-glyceryl phos- | ARA_LEAF | p-Super | GC | 38 | 117 |
| | | | phate (pf) | | | | | |
| 44372 | non-targ | B3989 | triacylglycerol | ARA_LEAF | p-Super | LC | 33 | 109 |
| 10740 | non-targ | B4029 | bis-glyceryl phos- | ARA_LEAF | p-Super | GC | 38 | 220 |
| | | | phate (pf) | | | | | |
| 106532 | non-targ | B4050 | triacylglycerol | ARA_LEAF | p-Super | LC | 50 | 173 |
| 44662 | non-targ | B4129 | bis-glyceryl phos- | ARA_LEAF | p-Super | GC | 39 | 79 |
| | | | phate (pf) | | | | | |
| 45556 | non-targ | B4384 | glycerol-3- | ARA_SEED_2 | p-USP | LC | 45 | 83 |
| | | | phosphate (pf) | | | | | |
| 10811 | non-targ | GM02LC12622 | bis-glyceryl phos- | ARA_LEAF | p-PcUBI | GC | 41 | 98 |
| | | | phate (pf) | | | | | |
| 10811 | non-targ | GM02LC12622 | glycerol phosphate | ARA_LEAF | p-PcUBI | GC | 18 | 34 |
| | | | (lf) | | | | | |
| 46405 | non-targ | GM02LC17485 | bis-glyceryl phos- | ARA_LEAF | p-PcUBI | GC | 69 | 112 |
| | | | phate (pf) | | | | | |
| 46751 | non-targ | GM02LC19289 | glycerol (lf) | ARA_LEAF | p-PcUBI | GC | 25 | 59 |
| 46751 | non-targ | GM02LC19289 | glycerol phosphate | ARA_LEAF | p-PcUBI | GC | 18 | 82 |
| | | | (lf) | | | | | |
| 84198 | non-targ | GM02LC21368 | glycerol (pf) | ARA_LEAF | p-PcUBI | GC | 75 | 89 |
| 46850 | non-targ | GM02LC44512 | glycerol phosphate | ARA_LEAF | p-PcUBI | GC | 20 | 30 |
| | | | (lf) | | | | | |
| 54337 | mitoch | SII1450 | bis-glyceryl phos- | ARA_LEAF | p-PcUBI | GC | 41 | 70 |
| | | | phate (pf) | | | | | |
| 95047 | non-targ | SII1498 | glycerol phosphate | ARA_LEAF | p-PcUBI | GC | 18 | 32 |
| | | | (lf) | | | | | |
| 54452 | plastidic | SII1522 | glycerol (lf) | ARA_LEAF | p-PcUBI | GC | 16 | 49 |
| 85808 | mitoch | SII1815 | bis-glyceryl phos- | ARA_LEAF | p-PcUBI | GC | 49 | 68 |
| | | | phate (pf) | | | | | |
| 86447 | plastidic | SII1848 | glycerol (lf) | ARA_LEAF | p-PcUBI | GC | 19 | 55 |
| 11471 | mitoch | SII1917 | glycerol phosphate | ARA_LEAF | p-PcUBI | GC | 17 | 67 |
| | | | (lf) | | | | | |
| 11990 | non-targ | Slr0338 | glycerol (lf) | ARA_LEAF | p-PcUBI | GC | 20 | 28 |
| 11990 | plastidic | Slr0338 | glycerol phosphate | ARA_LEAF | p-PcUBI | GC | 18 | 39 |
| | | | (lf) | | | | | |
| 147407 | mitoch | Slr0905 | glycerol (pf) | ARA_LEAF | p-PcUBI | GC | 53 | 73 |
| 57734 | non-targ | Slr1096 | bis-glyceryl phos- | ARA_LEAF | p-PcUBI | GC | 39 | 68 |
| | | | phate (pf) | | | | | |
| 116341 | mitoch | Slr1099 | glycerol phosphate | ARA_LEAF | p-PcUBI | GC | 17 | 29 |
| | | | (lf) | | | | | |
| 116460 | mitoch | Slr1250 | glycerol phosphate | ARA_LEAF | p-PcUBI | GC | 21 | 46 |
| | | | (lf) | | | | | |
| 58324 | non-targ | Slr1312 | glycerol phosphate | ARA_LEAF | p-PcUBI | GC | 24 | 43 |
| | | | (lf) | | | | | |
| 58590 | non-targ | Slr1420 | glycerol phosphate | ARA_LEAF | p-PcUBI | GC | 20 | 35 |
| | | | (lf) | | | | | |
| 154137 | plastidic | Slr1467 | bis-glyceryl phos- | ARA_LEAF | p-PcUBI | GC | 57 | 97 |
| | | | phate (pf) | | | | | |
| 147457 | plastidic | Slr1743 | bis-glyceryl phos- | ARA_LEAF | p-PcUBI | GC | 42 | 88 |
| | | | phate (pf) | | | | | |
| 127521 | mitoch | Slr2035 | glycerol (lf) | ARA_LEAF | p-PcUBI | GC | 18 | 22 |
| 12341 | mitoch | Slr2072 | glycerol (lf) | ARA_LEAF | p-PcUBI | GC | 18 | 43 |
| 12341 | mitoch | Slr2072 | glycerol phosphate | ARA_LEAF | p-PcUBI | GC | 19 | 28 |
| | | | (lf) | ARA_LEAF | | | | |
| 136907 | plastidic | Smr0004 | bis-glyceryl phos- | | p-PcUBI | GC | 44 | 60 |
| | | | phate (pf) | | | | | |
| 60301 | non-targ | TTC0035 | triacylglycerol | ARA_LEAF | p-PcUBI | LC | 73 | 163 |
| 61553 | non-targ | TTC0917 | glycerol phosphate | ARA_LEAF | p-PcUBI | GC | 21 | 73 |
| | | | (lf) | | | | | |
| 62079 | non-targ | TTC1386 | glycerol (lf) | ARA_LEAF | p-PcUBI | GC | 23 | 34 |
| 108092 | non-targ | Yal013w | glycerol-3- | ARA_SEED_2 | p-PcUBI | LC | 136 | 212 |
| | | | phosphate (pf) | | | | | |
| 62524 | non-targ | Ybl021c | glycerol (lf) | ARA_LEAF | Big35S | GC | 17 | 23 |
| 119347 | plastidic | Ybr092c | glycerol phosphate | ARA_LEAF | p-Super | GC | 24 | 31 |
| | | | (lf) | | | | | |
| 62717 | non-targ | Ybr160w | glycerol (lf) | ARA_LEAF | Big35S | GC | 17 | 63 |
| 62717 | non-targ | Ybr160w | glycerol (pf) | ARA_LEAF | Big35S | GC | 54 | 85 |
| 63264 | plastidic | Ycr102c | bis-glyceryl phos- | ARA_LEAF | p-Super | GC | 42 | 81 |
| | | | phate (pf) | | | | | |
| 90159 | plastidic | Ydr273w | bis-glyceryl phos- | ARA_LEAF | p-Super | GC | 41 | 48 |
| | | | phate (pf) | | | | | |
| 63807 | non-targ | Yfl016c | glycerol phosphate | ARA_LEAF | Big35S | GC | 22 | 31 |
| | | | (lf) | | | | | |
| 64144 | non-targ | Yfl019c | triacylglycerol | ARA_LEAF | Big35S | LC | 36 | 110 |
| 130742 | plastidic | Ygl256w | bis-glyceryl phos- | ARA_LEAF | p-Super | GC | 42 | 93 |
| | | | phate (pf) | | | | | |
| 128655 | plastidic | Ygr286c | glycerol (lf) | ARA_LEAF | p-Super | GC | 16 | 43 |
| 64336 | plastidic | Yhr024c | triacylglycerol | ARA_LEAF | p-Super | LC | 52 | 138 |
| 154394 | plastidic | Yhr202w | bis-glyceryl phos- | ARA_LEAF | p-Super | GC | 36 | 46 |
| | | | phate (pf) | | | | | |
| 14769 | non-targ | Yil053w | glycerol (pf) | ARA_LEAF | Big35S | GC | 49 | 2038 |
| 149454 | plastidic | Yil083c | bis-glyceryl phos- | ARA_LEAF | p-Super | GC | 48 | 101 |
| | | | phate (pf) | | | | | |
| 154437 | non-targ | Yjl062w-a | glycerol phosphate | ARA_LEAF | Big35S | GC | 18 | 23 |
| | | | (lf) | | | | | |
| 64964 | plastidic | Yjl073w | bis-glyceryl phos- | ARA_LEAF | p-Super | GC | 33 | 36 |
| | | | phate (pf) | | | | | |
| 66274 | non-targ | Yjr153w | bis-glyceryl phos- | ARA_LEAF | p-PcUBI | GC | 45 | 131 |
| | | | phate (pf) | | | | | |
| 150603 | non-targ | Ylr025w | glycerol (pf) | ARA_LEAF | Big35S | GC | 62 | 120 |
| 154454 | plastidic | Ylr099c | bis-glyceryl phos- | ARA_LEAF | p-Super | GC | 297 | 648 |
| | | | phate (pf) | | | | | |
| 129065 | non-targ | Ylr210w | glycerol (lf) | ARA_LEAF | Big35S | GC | 17 | 25 |
| 129123 | plastidic | Ylr326w | glycerol (lf) | ARA_LEAF | p-Super | GC | 17 | 42 |
| 129123 | plastidic | Ylr326w | glycerol phosphate | ARA_LEAF | p-Super | GC | 26 | 46 |
| | | | (lf) | | | | | |
| 139173 | non-targ | Ynl007c | glycerol (lf) | ARA_LEAF | Big35S | GC | 17 | 57 |
| 129470 | non-targ | Ynl130c | glycerol (lf) | ARA_LEAF | Big35S | GC | 17 | 53 |
| 129574 | non-targ | Yor187w | glycerol (lf) | ARA_LEAF | Big35S | GC | 18 | 49 |
| 154477 | plastidic | Ypl162c | bis-glyceryl phos- | ARA_LEAF | p-Super | GC | 37 | 52 |
| | | | phate (pf) | | | | | |
| 140110 | non-targ | Ypr054w | glycerol (lf) | ARA_LEAF | Big35S | GC | 20 | 42 |
| 68413 | non-targ | Zm_4842_BE510522 | bis-glyceryl phos- | ARA_LEAF | p-PcUBI | GC | 46 | 72 |
| | | | phate (pf) | | | | | |
| 69 | non-targ | 49747384_SOYBEAN | bis-glyceryl phos- | ARA_LEAF | p-PcUBI | GC | 47 | 114 |
| | | | phate (pf) | | | | | |

[101120] Column 1 shows the SEQ ID NO, Column 2 shows the expression type (targeted or non-targeted), Column 3 shows the “gene name” (locus), Column 4 shows the metabolite analyzed, Column 5 indicates the A. thaliana source tissue analyzed, Column 6 indicates the used promoter for expression, Column 7 indicates the analytical method. Columns 8 and 9 show the minimum and the maximum increase of the analyzed metabolite (in percent) in comparison to the wild type (ratio_by _WT, given as percent increase).

[101121] The term “non-targ” in Table d, Column 2 which shows the expression type means “non-targeted”.

[101122] The term “mitoch” in Table d, Column 2 which shows the expression type means “mitochondrial”.

[101123] The term “pf ” in Table d, Column 4 which shows the metabolite means “polar fraction”.

[101124] The term “lf ” in Table d, Column 4 which shows the metabolite means “lipid fraction”.

[101125] When the analyses were repeated independently, all results proved to be significant.

[101126] [0505.1.7.19] to [0515.1.7.19] for the disclosure of theses paragraphs see [0505.1.7.7] to [0515.1.7.7] above

[101127] In a further embodiment, the present invention relates in paragraphs [0000.1.7.20.] to [0514.1.7.20.] to a further process for the production of the fine chemical hentriacontane as defined below and corresponding embodiments as described herein as follows.

[101128] for the disclosure of this paragraph see [0001.1.7.7] above

[101129] Waxes are organic compounds that are plastic near ambient temperatures, when melting, they give a low viscosity liquid. Waxes are insoluble in water but soluble in petroleum based solvent.

[101130] Waxes characteristically consist of a long alkyl chains. They tend to contain wax esters as major components, i.e. esters of long-chain fatty alcohols with long-chain fatty acids. The nature of the other lipid constituents can vary greatly with the source of the waxy material, but they include hydrocarbons, saturated, e.g. n-alkanes and unsaturated, sterol esters, aliphatic aldehydes, primary and secondary alcohols, diols, ketones, 6-diketones, triacylglycerols, and many more.

[101131] Waxes are biosynthesized by many plants or animals.

[101132] The best known animal wax is beeswax, but other insects secrete waxes. A major component of beeswax is the ester myricyl palmitate substance and used in constructing their honeycombs. Lanolin for example is a wax obtained from wool, consisting of esters of sterols.

[101133] Plants secrete especially in warm climates, waxes as a way to control evaporation and hydration. Plant leaf surfaces are coated with a thin layer of waxy material that forms the outer boundary of the cuticular membrane; it is the interface between the plant and the atmosphere. Plant cuticular waxes serve as a protective barrier against water loss, UV light, pathogens and insects and helps the plants to resist drought.

[101134] Waxes differ widely among plant species and among the organs and tissues of a single plant, attesting to the genetic diversity and developmental influences In addition, wax content and composition are affected by environmental conditions. Relatively high-humidity conditions, such as in tissue culture, suppress wax production and the photoperiod affects the chain length of wax components.

[101135] Cuticular waxes of plants are complex mixtures of primarily very long chain fatty acids (VLCFAs, >018), hydrocarbons like long-chain aliphatic hydrocarbons, containing alkanes, alcohols, aldehydes, ketones, esters, triterpenes, sterols, and flavonoids, the proportions of the major classes vary among plant species. Hydrocarbons are e.g. a prominent part in leaves of cuticular waxes of peas' and cabbages' leaves.

[101136] As plants cover much of the earth's surface, it seems likely that plant waxes are the most abundant of all natural lipids.

[101137] Although most natural waxes are esters, paraffin waxes are hydrocarbons, mixtures of alkanes of different chain lengths. These materials represent a significant fraction of petroleum. They are refined by vacuum distillation. Paraffin waxes are mixtures of saturated of n- and isoalkanes, naphthenes, and alkyl- and naphthene-substituted aromatic compounds. They are used in adhesives, in foods (such as chewing gum and cheese wrapping), in cosmetics, and as coatings.

[101138] Montan wax is a fossilized wax extracted from coal and lignite. It is very hard, reflecting the high concentration of saturated fatty acids and alcohols, not esters that characterize softer waxes.

[101139] Although dark brown and smelly, they can be purified and bleached to give commercially useful products.

[101140] All the aliphatic components of plant waxes are synthesised in the epidermal cells from saturated very-long-chain fatty acids (commonly C20 to C34). 16:0 and 18:0 fatty acids are first synthesised in the stroma of plastids by the soluble enzymes forming the fatty acid synthase complex. The second stage involves multiple elongation steps and is catalysed by membrane-associated multi-enzyme complexes, known as fatty acid elongases outwith the plastids. As in fatty acid synthesis de novo, each two-carbon extension of the chain involves four reactions: condensation between a CoA-esterified fatty acyl substrate and malonyl-CoA, followed by a β-keto reduction, dehydration and an enoyl reduction to produce saturated verylong-chain fatty acids with 24 to 36 carbon atoms. The enzymes that catalyze these reactions of elongation of long-chain fatty acids are collectively referred to as elongases. Many different forms of the elongases have been identified, and these must interact in some manner to produce the chain-length specificity observed.

[101141] There are then two main pathways for biosynthesis of wax components: an acyl reduction pathway, which yields primary alcohols and wax esters, and a decarbonylation pathway that results in synthesis of aldehydes, alkanes, secondary alcohols and ketones. In the reductive pathway, acyl-CoA esters produced by chain elongation are reduced in a two-step process via a transient aldehyde intermediate, catalysed by a single enzyme, an acyl-CoA reductase (though it was once thought that two distinct enzymes were involved).

[101142] In the decarbonylation pathway for the synthesis of wax constituents, the first step is again believed to be the reduction of acyl-CoA ester to an aldehyde by means of an acyl-CoA reductase. Removal of the carbonyl group by an aldehyde decarbonylase yields an alkane, with one fewer carbon atom than the fatty acid precursor.

[101143] Hentriacontane, H₃C—(CH₂)₂₉—CH₃, is a typical member of the group of C₂₀-C₃₄ straight-chain aliphatics found in plant wax.

[101144] Waxes have long been use to make candles. Today, waxes are mainly consumed industrially as components of complex formulations, like coatings, formulation for plastics or paints. They are employed as release agents. They are also used as slip agents, e.g. in furniture, and corrosion resistance.

[101145] Waxes are used for impregnating and coating paper and card to waterproof but also leather and fabric. Waxes are also used in shoe polishes, wood polishes, and automotive polishes. In another context, waxes are used in cosmetics, like lipstick and mascara which are blends of various fats and waxes colored with pigments.

[101146] Due to the wide variety of uses of waxes or components thereof, specially long chain n-alkanes (C20 to C40), preferably C25 to C35, more preferably hentriacontane, an increased production very important.

[101147] Particularly economical methods are biotechnological methods utilizing long chain n-alkanes, preferably hentriacontane-producing organisms which are either natural or optimized by genetic modification.

[101148] There is a constant need for providing novel enzyme activities or direct or indirect regulators and thus alternative methods with advantageous properties for producing long chain n-alkanes (C20 to C40) or derivates like alcohols and acid of alkanes, optionally unsaturated as well as break down and/or cracking products of long chain alkanes, preferably long chain alkanes of C25 to C35, more preferably hentriacontane or waxes or components thereof in organisms, e.g. in transgenic organisms.

[101149] The content of waxes or components thereof in a cell was also increased by increasing or decreasing certain activities as disclosed in WO2006/069610, WO2007/087815 or WO2008/034648

[101150] Improving the quality of foodstuffs and animal feeds is an important task of the food-and-feed industry. For this purpose a great number of derivates of, long chain n-alkanes, which occur in plants are limited with regard to the supply of mammals. Such derivates are alcohols and acid of alkanes, optionally unsaturated as well as break down and/or cracking products of long chain alkanes. Especially advantageous for the quality of foodstuffs and animal feeds is as balanced as possible a profile of the componenets, since a great excess of some component above a specific concentration in the food has no further positive effect on the utilization of the food since others suddenly become limiting. A further increase in quality is only possible via addition of further components, which are limiting under these conditions. The targeted addition of the limiting component in the form of synthetic products must be carried out with extreme caution in order to avoid imbalance. To ensure a high quality of foods and animal feeds, it is therefore necessary to add a plurality of components in a balanced manner to suit the respective organism.

[101151] Accordingly, there is still a great demand for new and more suitable genes, which encode enzymes or regulators, which participate in the biosynthesis of long chain n-alkanes (C20 to C40) or derivates like alcohols and acid of alkanes, optionally unsaturated as well as break down and/or cracking products of long chain alkanes, preferably long chain alkanea of C25 to C35, more preferably hentriacontane or waxes or components thereof and make it possible to produce certain long chain n-alkanes (C20 to C40) or derivates like alcohols and acid of alkanes, optionally unsaturated as well as break down and/or cracking products of long chain alkanes, preferably long chain alkanes of C25 to C35, more preferably hentriacontane or waxes or components thereof specifically on an industrial scale without unwanted byproducts being formed. In the selection of genes for biosynthesis or regulation two characteristics above all are particularly important. On the one hand, there is as ever a need for improved processes for obtaining the highest possible contents of long chain n-alkanes (C20 to C40) or derivates like alcohols and acid of alkanes, optionally unsaturated as well as break down and/or cracking products of long chain alkanes, preferably long chain alkanes of C25 to C35, more preferably hentriacontane or waxes or components thereof and on the other hand as less as possible byproducts should be produced in the production process.

[101152] Very long chain alkanes, as found amongst plant wax compounds, can be generated by chemical synthesis and extracted from fossil sources with sufficient chemical diversity and at very low cost. Biotechnical approaches for hentriacontane production would therefore have to face strong competition. On the other hand, with ongoing depletion of fossil sources of hydrocarbons, plants with increased hydrocarbon (e.g. hentriacontane) content might become desirable as renewable hydrocarbon sources.

[101153] It is an object of the present invention to develop an inexpensive process for the synthesis of hentriacontane.

[101154] It is a further object of the present invention to develop an inexpensive process for the synthesis of waxes or components thereof like long chain n-alkanes (C20 to C40) or derivates like alcohols and acid of these alkanes, optionally unsaturated as well as break down and/or cracking products of long chain alkanes, preferably long chain alkanes of C25 to C35, more preferably hentriacontane and to assure that the waxes or components thereof like long chain n-alkanes are more accessible and facilely to isolate and recover in an industrial scale from the producing organism, preferably from a plant.

[101155] for the disclosure of this paragraph see [0013.1.7.7] above

[101156] Accordingly, in a first embodiment, the invention relates to a process for the production of at least one fine chemical selected from the group consisting of: hentriacontane, or, in other words, of the “fine chemical” or “fine chemical of the invention”.

[101157] The terms “fine chemical of the invention”, “fine chemical” or “the fine chemical” are used herein equally and relate in context of the paragraphs or sections [0014.1.20.20] to [0514.1.7.20] essentially to the metabolite or the metabolites indicated in column 7, application no. 20 of Tables I, II and IV in the respective line.

[101158] Further, the term “in context of any of the paragraphs [0014.1.20.20] to [0514.1.7.20]” as used herein means that for any of said paragraphs [0014.1.20.20] to [0514.1.7.20] the term “the fine chemical” is understood to follow the definition of paragraphs or sections [0014.1.20.20] and [0015.1.20.20], independently whether it refers to any other paragraph or not and whether the reference recites the term “fine chemical” in an other context. Thus, in cases where one or more paragraphs or sections are incorporated by reference into any of the present paragraphs [0014.1.20.20] to [0514.1.7.20], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to the definition of paragraph [0014.1.20.20] and [0015.1.20.20].

[101159] Accordingly, in one embodiment, the term “fine chemical of the invention”, “fine chemical” or “the fine chemical” as used herein (alone or in combination, like in FCRP) means “hentriacontane” in context of the nucleic acid or polypeptide sequences listed in the respective same line of any one of Tables I to IV of application no. 20 and indicating in column 7 the metabolite “hentriacontane”.

[101160] In one embodiment, the term hentriacontane or the term “fine chemical” mean in context of the paragraphs or sections [0014.1.20.20] to [0514.1.7.20] at least one chemical compound with an activity of the above mentioned hentriacontane, respectively.

[101161] Accordingly, in one embodiment, the terms “fine chemical of the invention”, “fine chemical” or “the fine chemical” mean in context of any of the paragraphs [0014.1.20.20] to [0514.1.7.20] hentriacontane, its derivates like alcohols and acid of these alkanes, optionally unsaturated as well as break down and/or cracking products in free form or bound form. In a preferred embodiment, the term “the fine chemical” means hentriacontane or its derivates like alcohols and acid of these alkanes, optionally unsaturated as well as break down and/or cracking products, in free form or bound form.

[101162] In a preferred embodiment “hentriacontane” means hentriacontane in free form.

[101163] Further, the term “in context of any of the paragraphs [0014.1.20.20] to [0514.1.7.20]” as used herein means that for any of said paragraphs [0014.1.20.20] to [0514.1.7.20] the term “the fine chemical” is understood to follow the definition of section [0014.1.20.20] or section [0015.1.20.20], independently whether it refers to any other paragraph or not and whether the reference recites the term “fine chemical” in an other context.

[101164] Thus, in cases where one or more paragraphs or sections are incorporated by reference into any of the present paragraphs [0014.1.20.20] to [0514.1.7.20], e.g. by usage of the term “see paragraph” or the term “for the disclosure of this paragraph see the disclosure of paragraph” or the term “incorporated by reference” or a corresponding term, the incorporated paragraph, section or term “the fine chemical” is also understood to have the meaning according to this definition of this paragraph [0015.1.20.20].

[101165] Further, the term “fine chemicals” as used herein relates to compositions comprising said fine chemical(s), i.e. comprising hentriacontane, respectively.

[101166] Further, the term “fine chemicals” as used herein relates in one embodiment to waxes or components thereof like long chain n-alkanes (C20 to C40) or derivates like alcohols and acid of these alkanes, optionally unsaturated as well as break down and/or cracking products of long chain alkanes, preferably long chain alkanes of C25 to C35 (C₂₅ Pentacosane, C₂₆ Hexacosane, C₂₇ Heptacosane, C₂₈ Octacosane, C₂₉ Nonacosane, C30 Triacontane, C₃₁ Hentriacontane, C₃₂ Dotriacontane, C₃₃ Tritriacontane, C₃₄ Tetratriacontane, C₃₅ Pentatriacontane), more preferably hentriacontane.

[101167] Accordingly, the present invention relates to a process for the production of hentriacontane, which comprises
[101168] (a) increasing or generating one or more activities selected from the group consisting of pyruvate kinase in a non-human organism or a part thereof, preferably a microorganism, a plant cell, a plant or a part thereof, as compared to a corresponding non-transformed wild type non-human organism or a part thereof; and
[101169] (b) growing the non-human organism or a part thereof under conditions which permit the production of hentriacontane or a composition comprising hentriacontane in said non-human organism or in the culture medium surrounding said non-human organism.

[101170] Accordingly, the present invention relates to a process for the production of hentriacontane, which comprises
[101171] (a) increasing or generating one or more activities selected from the group consisting of MADS box protein transcription factor in a non-human organism or a part thereof, preferably a microorganism, a plant cell, a plant or a part thereof, as compared to a corresponding non-transformed wild type non-human organism or a part thereof; and
[101172] (b) growing the non-human organism or a part thereof under conditions which permit the production of hentriacontane or a composition comprising hentriacontane in said non-human organism or in the culture medium surrounding said non-human organism.

[101173] Accordingly, the present invention relates to a process for the production of hentriacontane, which comprises
[101174] (a) increasing or generating one or more activities selected from the group consisting of At2g45420-protein in a non-human organism or a part thereof, preferably a microorganism, a plant cell, a plant or a part thereof, as compared to a corresponding non-transformed wild type non-human organism or a part thereof; and
[101175] (b) growing the non-human organism or a part thereof under conditions which permit the production of hentriacontane or a composition comprising hentriacontane in said non-human organism or in the culture medium surrounding said non-human organism.

[101176] Accordingly, the present invention relates to a process for the production of hentriacontane, which comprises
[101177] (a) increasing or generating one or more activities selected from the group consisting of transcription factor in a non-human organism or a part thereof, preferably a microorganism, a plant cell, a plant or a part thereof, as compared to a corresponding non-transformed wild type non-human organism or a part thereof; and
[101178] (b) growing the non-human organism or a part thereof under conditions which permit the production of hentriacontane or a composition comprising hentriacontane in said non-human organism or in the culture medium surrounding said non-human organism.

[101179] Accordingly, the present invention relates to a process for the production of hentriacontane, which comprises
[101180] (a) increasing or generating one or more activities selected from the group consisting of At5g43630-protein in a non-human organism or a part thereof, preferably a microorganism, a plant cell, a plant or a part thereof, as compared to a corresponding non-transformed wild type non-human organism or a part thereof; and
[101181] (b) growing the non-human organism or a part thereof under conditions which permit the production of hentriacontane or a composition comprising hentriacontane in said non-human organism or in the culture medium surrounding said non-human organism.

[101182] Accordingly, the present invention relates to a process for the production of hentriacontane, which comprises
[101183] (a) increasing or generating one or more activities selected from the group consisting of RNA-binding protein in a non-human organism or a part thereof, preferably a microorganism, a plant cell, a plant or a part thereof, as compared to a corresponding non-transformed wild type non-human organism or a part thereof; and
[101184] (b) growing the non-human organism or a part thereof under conditions which permit the production of hentriacontane or a composition comprising hentriacontane in said non-human organism or in the culture medium surrounding said non-human organism.

[101185] Accordingly, the present invention relates to a process for the production of hentriacontane, which comprises
[101186] (a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
[101187] (b) growing the non-human organism or a part thereof under conditions which permit the production of hentriacontane or a composition comprising hentriacontane in said non-human organism or in the culture medium surrounding said non-human organism.

[101188] Accordingly, the present invention relates to a process for the production of hentriacontane, which comprises
[101189] (a) increasing or generating one or more activities selected from the group consisting of fumarylacetoacetate hydrolase in a non-human organism or a part thereof, preferably a microorganism, a plant cell, a plant or a part thereof, as compared to a corresponding non-transformed wild type non-human organism or a part thereof; and
[101190] (b) growing the non-human organism or a part thereof under conditions which permit the production of hentriacontane or a composition comprising hentriacontane in said non-human organism or in the culture medium surrounding said non-human organism.

[101191] Accordingly, the present invention relates to a process for the production of hentriacontane, which comprises
[101192] (a) increasing or generating one or more activities selected from the group consisting of spermidine synthase in a non-human organism or a part thereof, preferably a microorganism, a plant cell, a plant or a part thereof, as compared to a corresponding non-transformed wild type non-human organism or a part thereof; and
[101193] (b) growing the non-human organism or a part thereof under conditions which permit the production of hentriacontane or a composition comprising hentriacontane in said non-human organism or in the culture medium surrounding said non-human organism.

[101194] Accordingly, the present invention relates to a process for the production of hentriacontane, which comprises
[101195] (a) increasing or generating one or more activities selected from the group consisting of serine protease in a non-human organism or a part thereof, preferably a microorganism, a plant cell, a plant or a part thereof, as compared to a corresponding non-transformed wild type non-human organism or a part thereof; and
[101196] (b) growing the non-human organism or a part thereof under conditions which permit the production of hentriacontane or a composition comprising hentriacontane in said non-human organism or in the culture medium surrounding said non-human organism.

[101197] Accordingly, the present invention relates to a process for the production of hentriacontane, which comprises
[101198] (a) increasing or generating one or more activities selected from the group consisting of peptide chain release factor in a non-human organism or a part thereof, preferably a microorganism, a plant cell, a plant or a part thereof, as compared to a corresponding non-transformed wild type non-human organism or a part thereof; and
[101199] (b) growing the non-human organism or a part thereof under conditions which permit the production of hentriacontane or a composition comprising hentriacontane in said non-human organism or in the culture medium surrounding said non-human organism.

[101200] Accordingly, the present invention relates to a process for the production of hentriacontane, which comprises
[101201] (a) increasing or generating one or more activities selected from the group consisting of mechanosensitive ion channel in a non-human organism or a part thereof, preferably a microorganism, a plant cell, a plant or a part thereof, as compared to a corresponding non-transformed wild type non-human organism or a part thereof; and
[101202] (b) growing the non-human organism or a part thereof under conditions which permit the production of hentriacontane or a composition comprising hentriacontane in said non-human organism or in the culture medium surrounding said non-human organism.

[101203] Accordingly, the present invention relates to a process for the production of hentriacontane, which comprises
[101204] (a) increasing or generating one or more activities selected from the group consisting of pyrimidine-specific ribonucleoside hydrolase in a non-human organism or a part thereof, preferably a microorganism, a plant cell, a plant or a part thereof, as compared to a corresponding non-transformed wild type non-human organism or a part thereof; and
[101205] (b) growing the non-human organism or a part thereof under conditions which permit the production of hentriacontane or a composition comprising hentriacontane in said non-human organism or in the culture medium surrounding said non-human organism.

[101206] Accordingly, the present invention relates to a process for the production of hentriacontane, which comprises
[101207] (a) increasing or generating one or more activities selected from the group consisting of inner membrane protein in a non-human organism or a part thereof, preferably a microorganism, a plant cell, a plant or a part thereof, as compared to a corresponding non-transformed wild type non-human organism or a part thereof; and
[101208] (b) growing the non-human organism or a part thereof under conditions which permit the production of hentriacontane or a composition comprising hentriacontane in said non-human organism or in the culture medium surrounding said non-human organism.

[101209] Accordingly, the present invention relates to a process for the production of hentriacontane, which comprises
[101210] (a) increasing or generating one or more activities selected from the group consisting of b0833-protein in a non-human organism or a part thereof, preferably a microorganism, a plant cell, a plant or a part thereof, as compared to a corresponding non-transformed wild type non-human organism or a part thereof; and
[101211] (b) growing the non-human organism or a part thereof under conditions which permit the production of hentriacontane or a composition comprising hentriacontane in said non-human organism or in the culture medium surrounding said non-human organism.

[101212] Accordingly, the present invention relates to a process for the production of hentriacontane, which comprises
[101213] (a) increasing or generating one or more activities selected from the group consisting of S-adenosyl-L-methionine-dependent methyltransferase in a non-human organism or a part thereof, preferably a microorganism, a plant cell, a plant or a part thereof, as compared to a corresponding non-transformed wild type non-human organism or a part thereof; and
[101214] (b) growing the non-human organism or a part thereof under conditions which permit the production of hentriacontane or a composition comprising hentriacontane in said non-human organism or in the culture medium surrounding said non-human organism.

[101215] Accordingly, the present invention relates to a process for the production of hentriacontane, which comprises
[101216] (a) increasing or generating one or more activities selected from the group consisting of b1259-protein in a non-human organism or a part thereof, preferably a microorganism, a plant cell, a plant or a part thereof, as compared to a corresponding non-transformed wild type non-human organism or a part thereof; and
[101217] (b) growing the non-human organism or a part thereof under conditions which permit the production of hentriacontane or a composition comprising hentriacontane in said non-human organism or in the culture medium surrounding said non-human organism.

[101218] Accordingly, the present invention relates to a process for the production of hentriacontane, which comprises
[101219] (a) increasing or generating one or more activities selected from the group consisting of orotidine-5′-phosphate decarboxylase in a non-human organism or a part thereof, preferably a microorganism, a plant cell, a plant or a part thereof, as compared to a corresponding non-transformed wild type non-human organism or a part thereof; and
[101220] (b) growing the non-human organism or a part thereof under conditions which permit the production of hentriacontane or a composition comprising hentriacontane in said non-human organism or in the culture medium surrounding said non-human organism.

[101221] Accordingly, the present invention relates to a process for the production of hentriacontane, which comprises
[101222] (a) increasing or generating one or more activities selected from the group consisting of ATP-dependent RNA helicase in a non-human organism or a part thereof, preferably a microorganism, a plant cell, a plant or a part thereof, as compared to a corresponding non-transformed wild type non-human organism or a part thereof; and
[101223] (b) growing the non-human organism or a part thereof under conditions which permit the production of hentriacontane or a composition comprising hentriacontane in said non-human organism or in the culture medium surrounding said non-human organism.

[101224] Accordingly, the present invention relates to a process for the production of hentriacontane, which comprises
[101225] (a) increasing or generating one or more activities selected from the group consisting of b1445-protein in a non-human organism or a part thereof, preferably a microorganism, a plant cell, a plant or a part thereof, as compared to a corresponding non-transformed wild type non-human organism or a part thereof; and
[101226] (b) growing the non-human organism or a part thereof under conditions which permit the production of hentriacontane or a composition comprising hentriacontane in said non-human organism or in the culture medium surrounding said non-human organism.

[101227] Accordingly, the present invention relates to a process for the production of hentriacontane, which comprises
[101228] (a) increasing or generating one or more activities selected from the group consisting of acid shock protein in a non-human organism or a part thereof, preferably a microorganism, a plant cell, a plant or a part thereof, as compared to a corresponding non-transformed wild type non-human organism or a part thereof; and
[101229] (b) growing the non-human organism or a part thereof under conditions which permit the production of hentriacontane or a composition comprising hentriacontane in said non-human organism or in the culture medium surrounding said non-human organism.

[101230] Accordingly, the present invention relates to a process for the production of hentriacontane, which comprises
[101231] (a) increasing or generating one or more activities selected from the group consisting of electron transport complex protein in a non-human organism or a part thereof, preferably a microorganism, a plant cell, a plant or a part thereof, as compared to a corresponding non-transformed wild type non-human organism or a part thereof; and
[101232] (b) growing the non-human organism or a part thereof under conditions which permit the production of hentriacontane or a composition comprising hentriacontane in said non-human organism or in the culture medium surrounding said non-human organism.

[101233] Accordingly, the present invention relates to a process for the production of hentriacontane, which comprises
[101234] (a) increasing or generating one or more activities selected from the group consisting of transcriptional regulator in a non-human organism or a part thereof, preferably a microorganism, a plant cell, a plant or a part thereof, as compared to a corresponding non-transformed wild type non-human organism or a part thereof; and
[101235] (b) growing the non-human organism or a part thereof under conditions which permit the production of hentriacontane or a composition comprising hentriacontane in said non-human organism or in the culture medium surrounding said non-human organism.

[101236] Accordingly, the present invention relates to a process for the production of hentriacontane, which comprises
[101237] (a) increasing or generating one or more activities selected from the group consisting of b1670-protein in a non-human organism or a part thereof, preferably a microorganism, a plant cell, a plant or a part thereof, as compared to a corresponding non-transformed wild type non-human organism or a part thereof; and
[101238] (b) growing the non-human organism or a part thereof under conditions which permit the production of hentriacontane or a composition comprising hentriacontane in said non-human organism or in the culture medium surrounding said non-human organism.

[101239] Accordingly, the present invention relates to a process for the production of hentriacontane, which comprises
[101240] (a) increasing or generating one or more activities selected from the group consisting of phospho-2-dehydro-3-deoxyheptonate aldolase in a non-human organism or a part thereof, preferably a microorganism, a plant cell, a plant or a part thereof, as compared to a corresponding non-transformed wild type non-human organism or a part thereof; and
[101241] (b) growing the non-human organism or a part thereof under conditions which permit the production of hentriacontane or a composition comprising hentriacontane in said non-human organism or in the culture medium surrounding said non-human organism.

[101242] Accordingly, the present invention relates to a process for the production of hentriacontane, which comprises
[101243] (a) increasing or generating one or more activities selected from the group consisting of b1930-protein in a non-human organism or a part thereof, preferably a microorganism, a plant cell, a plant or a part thereof, as compared to a corresponding non-transformed wild type non-human organism or a part thereof; and
[101244] (b) growing the non-human organism or a part thereof under conditions which permit the production of hentriacontane or a composition comprising hentriacontane in said non-human organism or in the culture medium surrounding said non-human organism.

[101245] Accordingly, the present invention relates to a process for the production of hentriacontane, which comprises
[101246] (a) increasing or generating one or more activities selected from the group consisting of O-antigen chain length determinant in a non-human organism or a part thereof, preferably a microorganism, a plant cell, a plant or a part thereof, as compared to a corresponding non-transformed wild type non-human organism or a part thereof; and
[101247] (b) growing the non-human organism or a part thereof under conditions which permit the production of hentriacontane or a composition comprising hentriacontane in said non-human organism or in the culture medium surrounding said non-human organism.

[101248] Accordingly, the present invention relates to a process for the production of hentriacontane, which comprises
[101249] (a) increasing or generating one or more activities selected from the group consisting of GDP-mannose 4,6-dehydratase in a non-human organism or a part thereof, preferably a microorganism, a plant cell, a plant or a part thereof, as compared to a corresponding non-transformed wild type non-human organism or a part thereof; and
[101250] (b) growing the non-human organism or a part thereof under conditions which permit the production of hentriacontane or a composition comprising hentriacontane in said non-human organism or in the culture medium surrounding said non-human organism.

[101251] Accordingly, the present invention relates to a process for the production of hentriacontane, which comprises
[101252] (a) increasing or generating one or more activities selected from the group consisting of assembly protein for periplasmic nitrate reductase in a non-human organism or a part thereof, preferably a microorganism, a plant cell, a plant or a part thereof, as compared to a corresponding non-transformed wild type non-human organism or a part thereof; and
[101253] (b) growing the non-human organism or a part thereof under conditions which permit the production of hentriacontane or a composition comprising hentriacontane in said non-human organism or in the culture medium surrounding said non-human organism.

[101254] Accordingly, the present invention relates to a process for the production of hentriacontane, which comprises
[101255] (a) increasing or generating one or more activities selected from the group consisting of ion-transport protein in a non-human organism or a part thereof, preferably a microorganism, a plant cell, a plant or a part thereof, as compared to a corresponding non-transformed wild type non-human organism or a part thereof; and
[101256] (b) growing the non-human organism or a part thereof under conditions which permit the production of hentriacontane or a composition comprising hentriacontane in said non-human organism or in the culture medium surrounding said non-human organism.

[101257] Accordingly, the present invention relates to a process for the production of hentriacontane, which comprises
[101258] (a) increasing or generating one or more activities selected from the group consisting of b2399-protein in a non-human organism or a part thereof, preferably a microorganism, a plant cell, a plant or a part thereof, as compared to a corresponding non-transformed wild type non-human organism or a part thereof; and
[101259] (b) growing the non-human organism or a part thereof under conditions which permit the production of hentriacontane or a composition comprising hentriacontane in said non-human organism or in the culture medium surrounding said non-human organism.

[101260] Accordingly, the present invention relates to a process for the production of hentriacontane, which comprises
[101261] (a) increasing or generating one or more activities selected from the group consisting of cysteine synthase A in a non-human organism or a part thereof, preferably a microorganism, a plant cell, a plant or a part thereof, as compared to a corresponding non-transformed wild type non-human organism or a part thereof; and
[101262] (b) growing the non-human organism or a part thereof under conditions which permit the production of hentriacontane or a composition comprising hentriacontane in said non-human organism or in the culture medium surrounding said non-human organism.

[101263] Accordingly, the present invention relates to a process for the production of hentriacontane, which comprises
[101264] (a) increasing or generating one or more activities selected from the group consisting of ethanolamine utilization protein in a non-human organism or a part thereof, preferably a microorganism, a plant cell, a plant or a part thereof, as compared to a corresponding non-transformed wild type non-human organism or a part thereof; and
[101265] (b) growing the non-human organism or a part thereof under conditions which permit the production of hentriacontane or a composition comprising hentriacontane in said non-human organism or in the culture medium surrounding said non-human organism.

[101266] Accordingly, the present invention relates to a process for the production of hentriacontane, which comprises
[101267] (a) increasing or generating one or more activities selected from the group consisting of sigma-54 dependent response regulator in a non-human organism or a part thereof, preferably a microorganism, a plant cell, a plant or a part thereof, as compared to a corresponding non-transformed wild type non-human organism or a part thereof; and
[101268] (b) growing the non-human organism or a part thereof under conditions which permit the production of hentriacontane or a composition comprising hentriacontane in said non-human organism or in the culture medium surrounding said non-human organism.

[101269] Accordingly, the present invention relates to a process for the production of hentriacontane, which comprises
[101270] (a) increasing or generating one or more activities selected from the group consisting of b2653-protein in a non-human organism or a part thereof, preferably a microorganism, a plant cell, a plant or a part thereof, as compared to a corresponding non-transformed wild type non-human organism or a part thereof; and
[101271] (b) growing the non-human organism or a part thereof under conditions which permit the production of hentriacontane or a composition comprising hentriacontane in said non-human organism or in the culture medium surrounding said non-human organism.

[101272] Accordingly, the present invention relates to a process for the production of hentriacontane, which comprises
[101273] (a) increasing or generating one or more activities selected from the group consisting of phosphoadenosine phosphosulfate reductase in a non-human organism or a part thereof, preferably a microorganism, a plant cell, a plant or a part thereof, as compared to a corresponding non-transformed wild type non-human organism or a part thereof; and
[101274] (b) growing the non-human organism or a part thereof under conditions which permit the production of hentriacontane or a composition comprising hentriacontane in said non-human organism or in the culture medium surrounding said non-human organism.

[101275] Accordingly, the present invention relates to a process for the production of hentriacontane, which comprises
[101276] (a) increasing or generating one or more activities selected from the group consisting of amino-acid acetyltransferase in a non-human organism or a part thereof, preferably a microorganism, a plant cell, a plant or a part thereof, as compared to a corresponding non-transformed wild type non-human organism or a part thereof; and
[101277] (b) growing the non-human organism or a part thereof under conditions which permit the production of hentriacontane or a composition comprising hentriacontane in said non-human organism or in the culture medium surrounding said non-human organism.

[101278] Accordingly, the present invention relates to a process for the production of hentriacontane, which comprises
[101279] (a) increasing or generating one or more activities selected from the group consisting of exonuclease subunit in a non-human organism or a part thereof, preferably a microorganism, a plant cell, a plant or a part thereof, as compared to a corresponding non-transformed wild type non-human organism or a part thereof; and
[101280] (b) growing the non-human organism or a part thereof under conditions which permit the production of hentriacontane or a composition comprising hentriacontane in said non-human organism or in the culture medium surrounding said non-human organism.

[101281] Accordingly, the present invention relates to a process for the production of hentriacontane, which comprises
[101282] (a) increasing or generating one or more activities selected from the group consisting of L-asparaginase in a non-human organism or a part thereof, preferably a microorganism, a plant cell, a plant or a part thereof, as compared to a corresponding non-transformed wild type non-human organism or a part thereof; and
[101283] (b) growing the non-human organism or a part thereof under conditions which permit the production of hentriacontane or a composition comprising hentriacontane in said non-human organism or in the culture medium surrounding said non-human organism.

[101284] Accordingly, the present invention relates to a process for the production of hentriacontane, which comprises
[101285] (a) increasing or generating one or more activities selected from the group consisting of ornithine decarboxylase in a non-human organism or a part thereof, preferably a microorganism, a plant cell, a plant or a part thereof, as compared to a corresponding non-transformed wild type non-human organism or a part thereof; and
[101286] (b) growing the non-human organism or a part thereof under conditions which permit the production of hentriacontane or a composition comprising hentriacontane in said non-human organism or in the culture medium surrounding said non-human organism.

[101287] Accordingly, the present invention relates to a process for the production of hentriacontane, which comprises
[101288] (a) increasing or generating one or more activities selected from the group consisting of phosphoglycolate phosphatase in a non-human organism or a part thereof, preferably a microorganism, a plant cell, a plant or a part thereof, as compared to a corresponding non-transformed wild type non-human organism or a part thereof; and
[101289] (b) growing the non-human organism or a part thereof under conditions which permit the production of hentriacontane or a composition comprising hentriacontane in said non-human organism or in the culture medium surrounding said non-human organism.

[101290] Accordingly, the present invention relates to a process for the production of hentriacontane, which comprises
[101291] (a) increasing or generating one or more activities selected from the group consisting of sugar transport system permease protein in a non-human organism or a part thereof, preferably a microorganism, a plant cell, a plant or a part thereof, as compared to a corresponding non-transformed wild type non-human organism or a part thereof; and
[101292] (b) growing the non-human organism or a part thereof under conditions which permit the production of hentriacontane or a composition comprising hentriacontane in said non-human organism or in the culture medium surrounding said non-human organism.

[101293] Accordingly, the present invention relates to a process for the production of hentriacontane, which comprises
[101294] (a) increasing or generating one or more activities selected from the group consisting of cyclin D in a non-human organism or a part thereof, preferably a microorganism, a plant cell, a plant or a part thereof, as compared to a corresponding non-transformed wild type non-human organism or a part thereof; and
[101295] (b) growing the non-human organism or a part thereof under conditions which permit the production of hentriacontane or a composition comprising hentriacontane in said non-human organism or in the culture medium surrounding said non-human organism.

[101296] Accordingly, the present invention relates to a process for the production of hentriacontane, which comprises
[101297] (a) increasing or generating one or more activities selected from the group consisting of short-chain alcohol dehydrogenase family in a non-human organism or a part thereof, preferably a microorganism, a plant cell, a plant or a part thereof, as compared to a corresponding non-transformed wild type non-human organism or a part thereof; and
[101298] (b) growing the non-human organism or a part thereof under conditions which permit the production of hentriacontane or a composition comprising hentriacontane in said non-human organism or in the culture medium surrounding said non-human organism.

[101299] Accordingly, the present invention relates to a process for the production of hentriacontane, which comprises
[101300] (a) increasing or generating one or more activities selected from the group consisting of cell division protein in a non-human organism or a part thereof, preferably a microorganism, a plant cell, a plant or a part thereof, as compared to a corresponding non-transformed wild type non-human organism or a part thereof; and
[101301] (b) growing the non-human organism or a part thereof under conditions which permit the production of hentriacontane or a composition comprising hentriacontane in said non-human organism or in the culture medium surrounding said non-human organism.

[101302] Accordingly, the present invention relates to a process for the production of hentriacontane, which comprises
[101303] (a) increasing or generating one or more activities selected from the group consisting of metal-dependent hydrolase in a non-human organism or a part thereof, preferably a microorganism, a plant cell, a plant or a part thereof, as compared to a corresponding non-transformed wild type non-human organism or a part thereof; and
[101304] (b) growing the non-human organism or a part thereof under conditions which permit the production of hentriacontane or a composition comprising hentriacontane in said non-human organism or in the culture medium surrounding said non-human organism.

[101305] Accordingly, the present invention relates to a process for the production of hentriacontane, which comprises
[101306] (a) increasing or generating one or more activities selected from the group consisting of 3-deoxy-7-phosphoheptulonate synthase in a non-human organism or a part thereof, preferably a microorganism, a plant cell, a plant or a part thereof, as compared to a corresponding non-transformed wild type non-human organism or a part thereof; and
[101307] (b) growing the non-human organism or a part thereof under conditions which permit the production of hentriacontane or a composition comprising hentriacontane in said non-human organism or in the culture medium surrounding said non-human organism.

[101308] Accordingly, the present invention relates to a process for the production of hentriacontane, which comprises
[101309] (a) increasing or generating one or more activities selected from the group consisting of YFL019C-protein in a non-human organism or a part thereof, preferably a microorganism, a plant cell, a plant or a part thereof, as compared to a corresponding non-transformed wild type non-human organism or a part thereof; and
[101310] (b) growing the non-human organism or a part thereof under conditions which permit the production of hentriacontane or a composition comprising hentriacontane in said non-human organism or in the culture medium surrounding said non-human organism.

[101311] Accordingly, the present invention relates to a process for the production of hentriacontane, which comprises
[101312] (a) increasing or generating one or more activities selected from the group consisting of dihydroxyacetone kinase in a non-human organism or a part thereof, preferably a microorganism, a plant cell, a plant or a part thereof, as compared to a corresponding non-transformed wild type non-human organism or a part thereof; and
[101313] (b) growing the non-human organism or a part thereof under conditions which permit the production of hentriacontane or a composition comprising hentriacontane in said non-human organism or in the culture medium surrounding said non-human organism.

[101314] Accordingly, the present invention relates to a process for the production of hentriacontane, which comprises
[101315] (a) increasing or generating one or more activities selected from the group consisting of methionine permease in a non-human organism or a part thereof, preferably a microorganism, a plant cell, a plant or a part thereof, as compared to a corresponding non-transformed wild type non-human organism or a part thereof; and
[101316] (b) growing the non-human organism or a part thereof under conditions which permit the production of hentriacontane or a composition comprising hentriacontane in said non-human organism or in the culture medium surrounding said non-human organism.

[101317] Accordingly, the present invention relates to a process for the production of hentriacontane, which comprises
[101318] (a) increasing or generating one or more activities selected from the group consisting of YHL039W-protein in a non-human organism or a part thereof, preferably a microorganism, a plant cell, a plant or a part thereof, as compared to a corresponding non-transformed wild type non-human organism or a part thereof; and
[101319] (b) growing the non-human organism or a part thereof under conditions which permit the production of hentriacontane or a composition comprising hentriacontane in said non-human organism or in the culture medium surrounding said non-human organism.

[101320] Accordingly, the present invention relates to a process for the production of hentriacontane, which comprises
[101321] (a) increasing or generating one or more activities selected from the group consisting of YHR022C-A-protein in a non-human organism or a part thereof, preferably a microorganism, a plant cell, a plant or a part thereof, as compared to a corresponding non-transformed wild type non-human organism or a part thereof; and
[101322] (b) growing the non-human organism or a part thereof under conditions which permit the production of hentriacontane or a composition comprising hentriacontane in said non-human organism or in the culture medium surrounding said non-human organism.

[101323] Accordingly, the present invention relates to a process for the production of hentriacontane, which comprises
[101324] (a) increasing or generating one or more activities selected from the group consisting of YJL127W-A-protein in a non-human organism or a part thereof, preferably a microorganism, a plant cell, a plant or a part thereof, as compared to a corresponding non-transformed wild type non-human organism or a part thereof; and
[101325] (b) growing the non-human organism or a part thereof under conditions which permit the production of hentriacontane or a composition comprising hentriacontane in said non-human organism or in the culture medium surrounding said non-human organism.

[101326] Accordingly, the present invention relates to a process for the production of hentriacontane, which comprises
[101327] (a) increasing or generating one or more activities selected from the group consisting of ylr157c-protein in a non-human organism or a part thereof, preferably a microorganism, a plant cell, a plant or a part thereof, as compared to a corresponding non-transformed wild type non-human organism or a part thereof; and
[101328] (b) growing the non-human organism or a part thereof under conditions which permit the production of hentriacontane or a composition comprising hentriacontane in said non-human organism or in the culture medium surrounding said non-human organism.

[101329] Accordingly, the present invention relates to a process for the production of hentriacontane, which comprises
[101330] (a) increasing or generating one or more activities selected from the group consisting of ynl321w-protein in a non-human organism or a part thereof, preferably a microorganism, a plant cell, a plant or a part thereof, as compared to a corresponding non-transformed wild type non-human organism or a part thereof; and
[101331] (b) growing the non-human organism or a part thereof under conditions which permit the production of hentriacontane or a composition comprising hentriacontane in said non-human organism or in the culture medium surrounding said non-human organism.

[101332] Accordingly, the present invention relates to a process for the production of hentriacontane, which comprises
[101333] (a) increasing or generating one or more activities selected from the group consisting of ypr170c-protein in a non-human organism or a part thereof, preferably a microorganism, a plant cell, a plant or a part thereof, as compared to a corresponding non-transformed wild type non-human organism or a part thereof; and
[101334] (b) growing the non-human organism or a part thereof under conditions which permit the production of hentriacontane or a composition comprising hentriacontane in said non-human organism or in the culture medium surrounding said non-human organism.

[101335] Accordingly, the present invention relates to a process for the production of hentriacontane, which comprises
[101336] (a) increasing or generating one or more activities selected from the group consisting of YPR172W-protein in a non-human organism or a part thereof, preferably a microorganism, a plant cell, a plant or a part thereof, as compared to a corresponding non-transformed wild type non-human organism or a part thereof; and
[101337] (b) growing the non-human organism or a part thereof under conditions which permit the production of hentriacontane or a composition comprising hentriacontane in said non-human organism or in the culture medium surrounding said non-human organism.

[101338] Accordingly, the present invention relates to a process for the production of hentriacontane, which comprises
[101339] (a) increasing or generating one or more activities selected from the group consisting of b3644-protein in a non-human organism or a part thereof, preferably a microorganism, a plant cell, a plant or a part thereof, as compared to a corresponding non-transformed wild type non-human organism or a part thereof; and
[101340] (b) growing the non-human organism or a part thereof under conditions which permit the production of hentriacontane or a composition comprising hentriacontane in said non-human organism or in the culture medium surrounding said non-human organism.

[101341] An embodiment of the present invention relates to a process for the production of hentriacontane, which comprises
[101342] (a1) increasing or generating the activity of a polypeptide comprising a polypeptide as depicted in a respective line in column 5 or 8 of Table II, application no. 20, or a homolog or a fragment thereof, a consensus sequence or at least one polypeptide motif as depicted in the respective line in column 8 of Table IV, application no. 20, whereby the respective line disclose in column 7 the fine chemical hentriacontane; or
[101343] (a2) increasing or generating the activity of an expression product of one or more nucleic acid molecule(s) comprising a polynucleotide as depicted in the respective line in column 5 or 8 of Table I, application no. 20, preferably the coding region thereof, or a homolog or a fragment thereof, whereby the respective line disclose in column 7 the fine chemical hentriacontane; or
[101344] (a3) increasing or generating the activity of a functional equivalent of (a1) or (a2);
[101345] in a non-human organism or a part thereof; preferably a microorganism, a plant cell, a plant or a part thereof, as compared to a corresponding non-transformed wild type non-human organism or a part thereof; and
[101346] (b) growing the non-human organism under conditions which permit the production of hentriacontane or a composition comprising hentriacontane in said non-human organism or in the culture medium surrounding said non-human organism.

[101347] Accordingly, the increase or generation of one or more said activities is for example conferred by one or more expression products of said nucleic acid molecule, e.g. proteins. Accordingly, in the present invention described above, the increase or generation of one or more said activities is for example conferred by one or more protein(s) each comprising a polypeptide selected from the group as depicted in Table II, application no. 20, column 5 and 8, or a homolog or a fragment thereof.

[101348] The process of the invention comprises in one embodiment the following steps:
[101349] (i) increasing or generating of the expression of; and/or
[101350] (ii) increasing or generating the expression of an expression product of; and/or
[101351] (iii) increasing or generating one or more activities of an expression product encoded by;
   at least one nucleic acid molecule (in the following “Fine Chemical Related Protein (FCRP)”-encoding gene or “FCRP”-gene) comprising a nucleic acid molecule selected from the group consisting of:
[101352] (a) a nucleic acid molecule encoding the polypeptide shown in column 5 or 8 of Table II, preferably Table II B, application no. 20, or a homolog or a fragment thereof;
[101353] (b) a nucleic acid molecule shown in column 5 or 8 of Table I, preferably Table I B, application no. 20, or a homolog or a fragment thereof (preferably the coding region thereof);
[101354] (c) a nucleic acid molecule, which, as a result of the degeneracy of the genetic code, can be derived from a polypeptide sequence depicted in column 5 or 8 of Table II, preferably Table II B, application no. 20;
[101355] (d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 20, or the coding region thereof;
[101356] (e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
[101357] (f) a nucleic acid molecule which hybridizes with a nucleic acid molecule of (a), (b), (c), (d) or (e) under stringent hybridization conditions;
[101358] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
[101359] (h) a nucleic acid molecule encoding a polypeptide comprising the consensus sequence or one or more polypeptide motifs as shown in column 8 of Table IV, application no. 20;
[101360] (i) a nucleic acid molecule encoding a polypeptide having the activity represented by a respective protein as depicted in column 5 of Table II, application no. 20;
[101361] (j) a nucleic acid molecule which comprises a polynucleotide, which is obtained by amplifying a cDNA library or a genomic library using the primers as depicted in column 8 of Table III, application no. 20; and
[101362] (k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
[101363] or a nucleic acid molecule comprising a sequence which is complementary thereto.
   (All the references to Tables I to IV are references to the corresponding lines of Tables I to IV.)

[101364] In a preferred embodiment thereof said nucleic acid molecule encodes a polypeptide which has the activity of the polypeptide represented by a protein comprising a polypeptide as depicted in the corresponding hit in column 5 of Table II, application no. 20.

[101365] In another preferred embodiment thereof said nucleic acid molecule confers a generation or an increase of the respective fine chemical in a non-human organism or a part thereof as compared to a corresponding wild-type non-human organism or part thereof.

[101366] In another preferred embodiment thereof said nucleic acid molecule encodes a polypeptide which has the activity of the polypeptide represented by a protein as depicted in the corresponding hit in column 5 of Table II, application no. 20, and confers a generation or an increase of the respective fine chemical in a non-human organism or a part thereof as compared to a corresponding wild-type non-human organism or part thereof.

[101367] Accordingly, the genes of the present invention or used in accordance with the present invention, which respectively encode a protein having an activity of 3-deoxy-7-phosphoheptulonate synthase, acid shock protein, amino-acid acetyltransferase, assembly protein for periplasmic nitrate reductase, At2g45420-protein, At5g43630-protein, ATP-dependent RNA helicase, b0833-protein, b1259-protein, b1445-protein, b1670-protein, b1930-protein, b2399-protein, b2653-protein, b3644-protein, cell division protein, cyclin D, cysteine synthase A, dihydroxyacetone kinase, electron transport complex protein, ethanolamine utilization protein, exonuclease subunit, fumarylacetoacetate hydrolase, GDP-mannose 4,6-dehydratase, inner membrane protein, ion-transport protein, L-asparaginase, MADS box protein transcription factor, mechanosensitive ion channel, metal-dependent hydrolase, methionine permease, O-antigen chain length determinant, ornithine decarboxylase, orotidine-5′-phosphate decarboxylase, peptide chain release factor, phospho-2-dehydro-3-deoxyheptonate aldolase, phosphoadenosine phosphosulfate reductase, phosphoglycolate phosphatase, pyrimidine-specific ribonucleoside hydrolase, pyruvate kinase, RNA-binding protein, S-adenosyl-L-methionine-dependent methyltransferase, serine protease, short-chain alcohol dehydrogenase family, sigma-54 dependent response regulator, spermidine synthase, sugar transport system permease protein, transcription factor, transcriptional regulator, YFL019C-protein, YHL039W-protein, YHR022C-A-protein, YJL127W-A-protein, ylr157c-protein, ynl321w-protein, ypr170c-protein, or YPR172W-protein, which respectively encode a protein comprising a polypeptide encoded by a nucleic acid sequence as shown in Table I, application no. 20, column 5 or 8, (preferably the coding region thereof), or a homolog or a fragment thereof, which respectively encode a protein comprising a polypeptide as depicted in Table II, application no. 20, column 5 or 8, or a homolg or a fragment thereof, and/or which respectively can be amplified with the primer set shown in Table III, application no. 20, column 8, are also referred to as “FCRP genes”.

[101368] Proteins or polypeptides encoded by “FCRP-genes” are referred to as “Fine Chemical Related Proteins” or “FCRP”. For the purposes of the description of the present invention, the respective protein having an activity of 3-deoxy-7-phosphoheptulonate synthase, acid shock protein, amino-acid acetyltransferase, assembly protein for periplasmic nitrate reductase, At2g45420-protein, At5g43630-protein, ATP-dependent RNA helicase, b0833-protein, b1259-protein, b1445-protein, b1670-protein, b1930-protein, b2399-protein, b2653-protein, b3644-protein, cell division protein, cyclin D, cysteine synthase A, dihydroxyacetone kinase, electron transport complex protein, ethanolamine utilization protein, exonuclease subunit, fumarylacetoacetate hydrolase, GDP-mannose 4,6-dehydratase, inner membrane protein, ion-transport protein, L-asparaginase, MADS box protein transcription factor, mechanosensitive ion channel, metal-dependent hydrolase, methionine permease, O-antigen chain length determinant, ornithine decarboxylase, orotidine-5′-phosphate decarboxylase, peptide chain release factor, phospho-2-dehydro-3-deoxyheptonate aldolase, phosphoadenosine phosphosulfate reductase, phosphoglycolate phosphatase, pyrimidine-specific ribonucleoside hydrolase, pyruvate kinase, RNA-binding protein, S-adenosyl-L-methionine-dependent methyltransferase, serine protease, short-chain alcohol dehydrogenase family, sigma-54 dependent response regulator, spermidine synthase, sugar transport system permease protein, transcription factor, transcriptional regulator, YFL019C-protein, YHL039W-protein, YHR022C-A-protein, YJL127W-A-protein, ylr157c-protein, ynl321w-protein, ypr170c-protein, or YPR172W-protein, the respective protein comprising a polypeptide encoded by one or more respective nucleic acid sequences as shown in Table I, application no. 20, column 5 or 8, (preferably the coding region thereof), or a homolog or fragment thereof, the respective protein comprising a respective polypeptide as depicted in Table II, application no. 20, column 5 or 8, or a homolog or fragment thereof, the respective protein comprising a sequence corresponding to the consensus sequence as shown in Table IV, application no. 20, column 8, and/or the respective protein comprising at least one polypeptide motif as shown in Table IV, application no. 20, column 8 is also referred to as Fine Chemical Related Protein” or “FCRP”.

[101369] Thus, in one embodiment, the present invention provides a process of the production of hentriacontane, by increasing or generating one or more activities, especially selected from the group consisting of 3-deoxy-7-phosphoheptulonate synthase, acid shock protein, amino-acid acetyltransferase, assembly protein for periplasmic nitrate reductase, At2g45420-protein, At5g43630-protein, ATP-dependent RNA helicase, b0833-protein, b1259-protein, b1445-protein, b1670-protein, b1930-protein, b2399-protein, b2653-protein, b3644-protein, cell division protein, cyclin D, cysteine synthase A, dihydroxyacetone kinase, electron transport complex protein, ethanolamine utilization protein, exonuclease subunit, fumarylacetoacetate hydrolase, GDP-mannose 4,6-dehydratase, inner membrane protein, ion-transport protein, L-asparaginase, MADS box protein transcription factor, mechanosensitive ion channel, metal-dependent hydrolase, methionine permease, O-antigen chain length determinant, ornithine decarboxylase, orotidine-5′-phosphate decarboxylase, peptide chain release factor, phospho-2-dehydro-3-deoxyheptonate aldolase, phosphoadenosine phosphosulfate reductase, phosphoglycolate phosphatase, pyrimidine-specific ribonucleoside hydrolase, pyruvate kinase, RNA-binding protein, S-adenosyl-L-methionine-dependent methyltransferase, serine protease, short-chain alcohol dehydrogenase family, sigma-54 dependent response regulator, spermidine synthase, sugar transport system permease protein, transcription factor, transcriptional regulator, YFL019C-protein, YHL039W-protein, YHR022C-A-protein, YJL127W-A-protein, ylr157c-protein, ynl321w-protein, ypr170c-protein, and YPR172W-protein, which is conferred by one or more FCRPs or the gene product of one or more FCRP-genes, for example by the gene product of a nucleic acid sequences comprising a polynucleotide selected from the group as shown in Table I, application no. 20, column 5 or 8, (preferably by the coding region thereof), or a homolog or a fragment thereof, e.g. or by one or more proteins each comprising a polypeptide encoded by one or more nucleic acid sequences selected from the group as shown in Table I, application no. 20, column 5 or 8, (preferably by the coding region thereof), or a homolog or a fragment thereof, or by one or more protein(s) each comprising a polypeptide selected from the group as depicted in Table II, application no. 20, column 5 and 8, or a homolog thereof, or a protein comprising a sequence corresponding to the consensus sequence or comprising at least one polypeptide motif as shown in Table IV, application no. 20, column 8.

[101370] for the disclosure of this paragraph see [0025.1.7.7] above

[101371] In an embodiment, the process comprises increasing or generating the activity of one or more polypeptides having said activity, e.g. by generating or increasing the amount and/or specific activity in the cell or a compartment of a cell of one of more FCRP, especially selected from the group consisting of 3-deoxy-7-phosphoheptulonate synthase, acid shock protein, amino-acid acetyltransferase, assembly protein for periplasmic nitrate reductase, At2g45420-protein, At5g43630-protein, ATP-dependent RNA helicase, b0833-protein, b1259-protein, b1445-protein, b1670-protein, b1930-protein, b2399-protein, b2653-protein, b3644-protein, cell division protein, cyclin D, cysteine synthase A, dihydroxyacetone kinase, electron transport complex protein, ethanolamine utilization protein, exonuclease subunit, fumarylacetoacetate hydrolase, GDP-mannose 4,6-dehydratase, inner membrane protein, ion-transport protein, L-asparaginase, MADS box protein transcription factor, mechanosensitive ion channel, metal-dependent hydrolase, methionine permease, O-antigen chain length determinant, ornithine decarboxylase, orotidine-5′-phosphate decarboxylase, peptide chain release factor, phospho-2-dehydro-3-deoxyheptonate aldolase, phosphoadenosine phosphosulfate reductase, phosphoglycolate phosphatase, pyrimidine-specific ribonucleoside hydrolase, pyruvate kinase, RNA-binding protein, S-adenosyl-L-methionine-dependent methyltransferase, serine protease, short-chain alcohol dehydrogenase family, sigma-54 dependent response regulator, spermidine synthase, sugar transport system permease protein, transcription factor, transcriptional regulator, YFL019C-protein, YHL039W-protein, YHR022C-A-protein, YJL127W-A-protein, ylr157c-protein, ynl321w-protein, ypr170c-protein, and YPR172W-protein, for example of the respective polypeptide as depicted in Table II, application no. 20, column 5 and 8, or a homolog or a fragment thereof, or the respective polypeptide comprising a sequence corresponding to the consensus sequences as shown in Table IV, application no. 20, column 8, or the respective polypeptide comprising at least one polypeptide motif as depicted in Table IV, application no. 20, column 8.

[101372] A further embodiment of the present invention relates to a process for the production of hentriacontane, which comprises
[101373] (a) increasing or generating the activity of a pyruvate kinase non-targeted in a non-human organism or a part thereof, preferably a microorganism, a plant cell, a plant or a part thereof, as compared to a corresponding non-transformed wild type non-human organism or a part thereof; and
[101374] (b) growing the non-human organism or a part thereof under conditions which permit the production of hentriacontane or a composition comprising hentriacontane in said non-human organism or in the culture medium surrounding said non-human organism.

[101375] A further embodiment of the present invention relates to a process for the production of hentriacontane, which comprises
[101376] (a) increasing or generating the activity of a MADS box protein transcription factor non-targeted in a non-human organism or a part thereof, preferably a microorganism, a plant cell, a plant or a part thereof, as compared to a corresponding non-transformed wild type non-human organism or a part thereof; and
[101377] (b) growing the non-human organism or a part thereof under conditions which permit the production of hentriacontane or a composition comprising hentriacontane in said non-human organism or in the culture medium surrounding said non-human organism.

[101378] A further embodiment of the present invention relates to a process for the production of hentriacontane, which comprises
[101379] (a) increasing or generating the activity of a At2g45420-protein non-targeted in a non-human organism or a part thereof, preferably a microorganism, a plant cell, a plant or a part thereof, as compared to a corresponding non-transformed wild type non-human organism or a part thereof; and
[101380] (b) growing the non-human organism or a part thereof under conditions which permit the production of hentriacontane or a composition comprising hentriacontane in said non-human organism or in the culture medium surrounding said non-human organism.

[101381] A further embodiment of the present invention relates to a process for the production of hentriacontane, which comprises
[101382] (a) increasing or generating the activity of a transcription factor non-targeted in a non-human organism or a part thereof, preferably a microorganism, a plant cell, a plant or a part thereof, as compared to a corresponding non-transformed wild type non-human organism or a part thereof; and
[101383] (b) growing the non-human organism or a part thereof under conditions which permit the production of hentriacontane or a composition comprising hentriacontane in said non-human organism or in the culture medium surrounding said non-human organism.

[101384] A further embodiment of the present invention relates to a process for the production of hentriacontane, which comprises
[101385] (a) increasing or generating the activity of a At5g43630-protein non-targeted in a non-human organism or a part thereof, preferably a microorganism, a plant cell, a plant or a part thereof, as compared to a corresponding non-transformed wild type non-human organism or a part thereof; and
[101386] (b) growing the non-human organism or a part thereof under conditions which permit the production of hentriacontane or a composition comprising hentriacontane in said non-human organism or in the culture medium surrounding said non-human organism.

[101387] A further embodiment of the present invention relates to a process for the production of hentriacontane, which comprises
[101388] (a) increasing or generating the activity of a RNA-binding protein non-targeted in a non-human organism or a part thereof, preferably a microorganism, a plant cell, a plant or a part thereof, as compared to a corresponding non-transformed wild type non-human organism or a part thereof; and
[101389] (b) growing the non-human organism or a part thereof under conditions which permit the production of hentriacontane or a composition comprising hentriacontane in said non-human organism or in the culture medium surrounding said non-human organism.

[101390] A further embodiment of the present invention relates to a process for the production of hentriacontane, which comprises
[101391] (a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
[101392] (b) growing the non-human organism or a part thereof under conditions which permit the production of hentriacontane or a composition comprising hentriacontane in said non-human organism or in the culture medium surrounding said non-human organism.

[101393] A further embodiment of the present invention relates to a process for the production of hentriacontane, which comprises
[101394] (a) increasing or generating the activity of a fumarylacetoacetate hydrolase non-targeted in a non-human organism or a part thereof, preferably a microorganism, a plant cell, a plant or a part thereof, as compared to a corresponding non-transformed wild type non-human organism or a part thereof; and
[101395] (b) growing the non-human organism or a part thereof under conditions which permit the production of hentriacontane or a composition comprising hentriacontane in said non-human organism or in the culture medium surrounding said non-human organism.

[101396] A further embodiment of the present invention relates to a process for the production of hentriacontane, which comprises
[101397] (a) increasing or generating the activity of a spermidine synthase non-targeted in a non-human organism or a part thereof, preferably a microorganism, a plant cell, a plant or a part thereof, as compared to a corresponding non-transformed wild type non-human organism or a part thereof; and
[101398] (b) growing the non-human organism or a part thereof under conditions which permit the production of hentriacontane or a composition comprising hentriacontane in said non-human organism or in the culture medium surrounding said non-human organism.

[101399] A further embodiment of the present invention relates to a process for the production of hentriacontane, which comprises
[101400] (a) increasing or generating the activity of a serine protease non-targeted in a non-human organism or a part thereof, preferably a microorganism, a plant cell, a plant or a part thereof, as compared to a corresponding non-transformed wild type non-human organism or a part thereof; and
[101401] (b) growing the non-human organism or a part thereof under conditions which permit the production of hentriacontane or a composition comprising hentriacontane in said non-human organism or in the culture medium surrounding said non-human organism.

[101402] A further embodiment of the present invention relates to a process for the production of hentriacontane, which comprises
[101403] (a) increasing or generating the activity of a peptide chain release factor non-targeted in a non-human organism or a part thereof, preferably a microorganism, a plant cell, a plant or a part thereof, as compared to a corresponding non-transformed wild type non-human organism or a part thereof; and
[101404] (b) growing the non-human organism or a part thereof under conditions which permit the production of hentriacontane or a composition comprising hentriacontane in said non-human organism or in the culture medium surrounding said non-human organism.

[101405] A further embodiment of the present invention relates to a process for the production of hentriacontane, which comprises
[101406] (a) increasing or generating the activity of a mechanosensitive ion channel non-targeted in a non-human organism or a part thereof, preferably a microorganism, a plant cell, a plant or a part thereof, as compared to a corresponding non-transformed wild type non-human organism or a part thereof; and
[101407] (b) growing the non-human organism or a part thereof under conditions which permit the production of hentriacontane or a composition comprising hentriacontane in said non-human organism or in the culture medium surrounding said non-human organism.

[101408] A further embodiment of the present invention relates to a process for the production of hentriacontane, which comprises
[101409] (a) increasing or generating the activity of a pyrimidine-specific ribonucleoside hydrolase non-targeted in a non-human organism or a part thereof, preferably a microorganism, a plant cell, a plant or a part thereof, as compared to a corresponding non-transformed wild type non-human organism or a part thereof; and
[101410] (b) growing the non-human organism or a part thereof under conditions which permit the production of hentriacontane or a composition comprising hentriacontane in said non-human organism or in the culture medium surrounding said non-human organism.

[101411] A further embodiment of the present invention relates to a process for the production of hentriacontane, which comprises
[101412] (a) increasing or generating the activity of a inner membrane protein non-targeted in a non-human organism or a part thereof, preferably a microorganism, a plant cell, a plant or a part thereof, as compared to a corresponding non-transformed wild type non-human organism or a part thereof; and
[101413] (b) growing the non-human organism or a part thereof under conditions which permit the production of hentriacontane or a composition comprising hentriacontane in said non-human organism or in the culture medium surrounding said non-human organism.

[101414] A further embodiment of the present invention relates to a process for the production of hentriacontane, which comprises
[101415] (a) increasing or generating the activity of a b0833-protein non-targeted in a non-human organism or a part thereof, preferably a microorganism, a plant cell, a plant or a part thereof, as compared to a corresponding non-transformed wild type non-human organism or a part thereof; and
[101416] (b) growing the non-human organism or a part thereof under conditions which permit the production of hentriacontane or a composition comprising hentriacontane in said non-human organism or in the culture medium surrounding said non-human organism.

[101417] A further embodiment of the present invention relates to a process for the production of hentriacontane, which comprises
[101418] (a) increasing or generating the activity of a S-adenosyl-L-methionine-dependent methyltransferase non-targeted in a non-human organism or a part thereof, preferably a microorganism, a plant cell, a plant or a part thereof, as compared to a corresponding non-transformed wild type non-human organism or a part thereof; and
[101419] (b) growing the non-human organism or a part thereof under conditions which permit the production of hentriacontane or a composition comprising hentriacontane in said non-human organism or in the culture medium surrounding said non-human organism.

[101420] A further embodiment of the present invention relates to a process for the production of hentriacontane, which comprises
[101421] (a) increasing or generating the activity of a b1259-protein non-targeted in a non-human organism or a part thereof, preferably a microorganism, a plant cell, a plant or a part thereof, as compared to a corresponding non-transformed wild type non-human organism or a part thereof; and
[101422] (b) growing the non-human organism or a part thereof under conditions which permit the production of hentriacontane or a composition comprising hentriacontane in said non-human organism or in the culture medium surrounding said non-human organism.

[101423] A further embodiment of the present invention relates to a process for the production of hentriacontane, which comprises
[101424] (a) increasing or generating the activity of a orotidine-5′-phosphate decarboxylase non-targeted in a non-human organism or a part thereof, preferably a microorganism, a plant cell, a plant or a part thereof, as compared to a corresponding non-transformed wild type non-human organism or a part thereof; and
[101425] (b) growing the non-human organism or a part thereof under conditions which permit the production of hentriacontane or a composition comprising hentriacontane in said non-human organism or in the culture medium surrounding said non-human organism.

[101426] A further embodiment of the present invention relates to a process for the production of hentriacontane, which comprises
[101427] (a) increasing or generating the activity of a ATP-dependent RNA helicase non-targeted in a non-human organism or a part thereof, preferably a microorganism, a plant cell, a plant or a part thereof, as compared to a corresponding non-transformed wild type non-human organism or a part thereof; and
[101428] (b) growing the non-human organism or a part thereof under conditions which permit the production of hentriacontane or a composition comprising hentriacontane in said non-human organism or in the culture medium surrounding said non-human organism.

[101429] A further embodiment of the present invention relates to a process for the production of hentriacontane, which comprises
[101430] (a) increasing or generating the activity of a b1445-protein non-targeted in a non-human organism or a part thereof, preferably a microorganism, a plant cell, a plant or a part thereof, as compared to a corresponding non-transformed wild type non-human organism or a part thereof; and
[101431] (b) growing the non-human organism or a part thereof under conditions which permit the production of hentriacontane or a composition comprising hentriacontane in said non-human organism or in the culture medium surrounding said non-human organism.

[101432] A further embodiment of the present invention relates to a process for the production of hentriacontane, which comprises
[101433] (a) increasing or generating the activity of a acid shock protein non-targeted in a non-human organism or a part thereof, preferably a microorganism, a plant cell, a plant or a part thereof, as compared to a corresponding non-transformed wild type non-human organism or a part thereof; and
[101434] (b) growing the non-human organism or a part thereof under conditions which permit the production of hentriacontane or a composition comprising hentriacontane in said non-human organism or in the culture medium surrounding said non-human organism.

[101435] A further embodiment of the present invention relates to a process for the production of hentriacontane, which comprises
[101436] (a) increasing or generating the activity of a electron transport complex protein non-targeted in a non-human organism or a part thereof, preferably a microorganism, a plant cell, a plant or a part thereof, as compared to a corresponding non-transformed wild type non-human organism or a part thereof; and
[101437] (b) growing the non-human organism or a part thereof under conditions which permit the production of hentriacontane or a composition comprising hentriacontane in said non-human organism or in the culture medium surrounding said non-human organism.

[101438] A further embodiment of the present invention relates to a process for the production of hentriacontane, which comprises
[101439] (a) increasing or generating the activity of a transcriptional regulator non-targeted in a non-human organism or a part thereof, preferably a microorganism, a plant cell, a plant or a part thereof, as compared to a corresponding non-transformed wild type non-human organism or a part thereof; and
[101440] (b) growing the non-human organism or a part thereof under conditions which permit the production of hentriacontane or a composition comprising hentriacontane in said non-human organism or in the culture medium surrounding said non-human organism.

[101441] A further embodiment of the present invention relates to a process for the production of hentriacontane, which comprises
[101442] (a) increasing or generating the activity of a b1670-protein non-targeted in a non-human organism or a part thereof, preferably a microorganism, a plant cell, a plant or a part thereof, as compared to a corresponding non-transformed wild type non-human organism or a part thereof; and
[101443] (b) growing the non-human organism or a part thereof under conditions which permit the production of hentriacontane or a composition comprising hentriacontane in said non-human organism or in the culture medium surrounding said non-human organism.

[101444] A further embodiment of the present invention relates to a process for the production of hentriacontane, which comprises
[101445] (a) increasing or generating the activity of a phospho-2-dehydro-3-deoxyheptonate aldolase non-targeted in a non-human organism or a part thereof, preferably a microorganism, a plant cell, a plant or a part thereof, as compared to a corresponding non-transformed wild type non-human organism or a part thereof; and
[101446] (b) growing the non-human organism or a part thereof under conditions which permit the production of hentriacontane or a composition comprising hentriacontane in said non-human organism or in the culture medium surrounding said non-human organism.

[101447] A further embodiment of the present invention relates to a process for the production of hentriacontane, which comprises
[101448] (a) increasing or generating the activity of a b1930-protein non-targeted in a non-human organism or a part thereof, preferably a microorganism, a plant cell, a plant or a part thereof, as compared to a corresponding non-transformed wild type non-human organism or a part thereof; and
[101449] (b) growing the non-human organism or a part thereof under conditions which permit the production of hentriacontane or a composition comprising hentriacontane in said non-human organism or in the culture medium surrounding said non-human organism.

[101450] A further embodiment of the present invention relates to a process for the production of hentriacontane, which comprises
[101451] (a) increasing or generating the activity of a O-antigen chain length determinant non-targeted in a non-human organism or a part thereof, preferably a microorganism, a plant cell, a plant or a part thereof, as compared to a corresponding non-transformed wild type non-human organism or a part thereof; and
[101452] (b) growing the non-human organism or a part thereof under conditions which permit the production of hentriacontane or a composition comprising hentriacontane in said non-human organism or in the culture medium surrounding said non-human organism.

[101453] A further embodiment of the present invention relates to a process for the production of hentriacontane, which comprises
[101454] (a) increasing or generating the activity of a GDP-mannose 4,6-dehydratase non-targeted in a non-human organism or a part thereof, preferably a microorganism, a plant cell, a plant or a part thereof, as compared to a corresponding non-transformed wild type non-human organism or a part thereof; and
[101455] (b) growing the non-human organism or a part thereof under conditions which permit the production of hentriacontane or a composition comprising hentriacontane in said non-human organism or in the culture medium surrounding said non-human organism.

[101456] A further embodiment of the present invention relates to a process for the production of hentriacontane, which comprises
[101457] (a) increasing or generating the activity of a assembly protein for periplasmic nitrate reductase non-targeted in a non-human organism or a part thereof, preferably a microorganism, a plant cell, a plant or a part thereof, as compared to a corresponding non-transformed wild type non-human organism or a part thereof; and
[101458] (b) growing the non-human organism or a part thereof under conditions which permit the production of hentriacontane or a composition comprising hentriacontane in said non-human organism or in the culture medium surrounding said non-human organism.

[101459] A further embodiment of the present invention relates to a process for the production of hentriacontane, which comprises
[101460] (a) increasing or generating the activity of a ion-transport protein non-targeted in a non-human organism or a part thereof, preferably a microorganism, a plant cell, a plant or a part thereof, as compared to a corresponding non-transformed wild type non-human organism or a part thereof; and
[101461] (b) growing the non-human organism or a part thereof under conditions which permit the production of hentriacontane or a composition comprising hentriacontane in said non-human organism or in the culture medium surrounding said non-human organism.

[101462] A further embodiment of the present invention relates to a process for the production of hentriacontane, which comprises
[101463] (a) increasing or generating the activity of a b2399-protein non-targeted in a non-human organism or a part thereof, preferably a microorganism, a plant cell, a plant or a part thereof, as compared to a corresponding non-transformed wild type non-human organism or a part thereof; and
[101464] (b) growing the non-human organism or a part thereof under conditions which permit the production of hentriacontane or a composition comprising hentriacontane in said non-human organism or in the culture medium surrounding said non-human organism.

[101465] A further embodiment of the present invention relates to a process for the production of hentriacontane, which comprises
[101466] (a) increasing or generating the activity of a cysteine synthase A non-targeted in a non-human organism or a part thereof, preferably a microorganism, a plant cell, a plant or a part thereof, as compared to a corresponding non-transformed wild type non-human organism or a part thereof; and
[101467] (b) growing the non-human organism or a part thereof under conditions which permit the production of hentriacontane or a composition comprising hentriacontane in said non-human organism or in the culture medium surrounding said non-human organism.

[101468] A further embodiment of the present invention relates to a process for the production of hentriacontane, which comprises
[101469] (a) increasing or generating the activity of a ethanolamine utilization protein non-targeted in a non-human organism or a part thereof, preferably a microorganism, a plant cell, a plant or a part thereof, as compared to a corresponding non-transformed wild type non-human organism or a part thereof; and
[101470] (b) growing the non-human organism or a part thereof under conditions which permit the production of hentriacontane or a composition comprising hentriacontane in said non-human organism or in the culture medium surrounding said non-human organism.

[101471] A further embodiment of the present invention relates to a process for the production of hentriacontane, which comprises
[101472] (a) increasing or generating the activity of a sigma-54 dependent response regulator non-targeted in a non-human organism or a part thereof, preferably a microorganism, a plant cell, a plant or a part thereof, as compared to a corresponding non-transformed wild type non-human organism or a part thereof; and
[101473] (b) growing the non-human organism or a part thereof under conditions which permit the production of hentriacontane or a composition comprising hentriacontane in said non-human organism or in the culture medium surrounding said non-human organism.

[101474] A further embodiment of the present invention relates to a process for the production of hentriacontane, which comprises
[101475] (a) increasing or generating the activity of a b2653-protein non-targeted in a non-human organism or a part thereof, preferably a microorganism, a plant cell, a plant or a part thereof, as compared to a corresponding non-transformed wild type non-human organism or a part thereof; and
[101476] (b) growing the non-human organism or a part thereof under conditions which permit the production of hentriacontane or a composition comprising hentriacontane in said non-human organism or in the culture medium surrounding said non-human organism.

[101477] A further embodiment of the present invention relates to a process for the production of hentriacontane, which comprises
[101478] (a) increasing or generating the activity of a phosphoadenosine phosphosulfate reductase non-targeted in a non-human organism or a part thereof, preferably a microorganism, a plant cell, a plant or a part thereof, as compared to a corresponding non-transformed wild type non-human organism or a part thereof; and
[101479] (b) growing the non-human organism or a part thereof under conditions which permit the production of hentriacontane or a composition comprising hentriacontane in said non-human organism or in the culture medium surrounding said non-human organism.

[101480] A further embodiment of the present invention relates to a process for the production of hentriacontane, which comprises
[101481] (a) increasing or generating the activity of a amino-acid acetyltransferase non-targeted in a non-human organism or a part thereof, preferably a microorganism, a plant cell, a plant or a part thereof, as compared to a corresponding non-transformed wild type non-human organism or a part thereof; and
[101482] (b) growing the non-human organism or a part thereof under conditions which permit the production of hentriacontane or a composition comprising hentriacontane in said non-human organism or in the culture medium surrounding said non-human organism.

[101483] A further embodiment of the present invention relates to a process for the production of hentriacontane, which comprises
[101484] (a) increasing or generating the activity of a exonuclease subunit non-targeted in a non-human organism or a part thereof, preferably a microorganism, a plant cell, a plant or a part thereof, as compared to a corresponding non-transformed wild type non-human organism or a part thereof; and
[101485] (b) growing the non-human organism or a part thereof under conditions which permit the production of hentriacontane or a composition comprising hentriacontane in said non-human organism or in the culture medium surrounding said non-human organism.

[101486] A further embodiment of the present invention relates to a process for the production of hentriacontane, which comprises
[101487] (a) increasing or generating the activity of a L-asparaginase non-targeted in a non-human organism or a part thereof, preferably a microorganism, a plant cell, a plant or a part thereof, as compared to a corresponding non-transformed wild type non-human organism or a part thereof; and
[101488] (b) growing the non-human organism or a part thereof under conditions which permit the production of hentriacontane or a composition comprising hentriacontane in said non-human organism or in the culture medium surrounding said non-human organism.

[101489] A further embodiment of the present invention relates to a process for the production of hentriacontane, which comprises
[101490] (a) increasing or generating the activity of a ornithine decarboxylase non-targeted in a non-human organism or a part thereof, preferably a microorganism, a plant cell, a plant or a part thereof, as compared to a corresponding non-transformed wild type non-human organism or a part thereof; and
[101491] (b) growing the non-human organism or a part thereof under conditions which permit the production of hentriacontane or a composition comprising hentriacontane in said non-human organism or in the culture medium surrounding said non-human organism.

[101492] A further embodiment of the present invention relates to a process for the production of hentriacontane, which comprises
[101493] (a) increasing or generating the activity of a phosphoglycolate phosphatase non-targeted in a non-human organism or a part thereof, preferably a microorganism, a plant cell, a plant or a part thereof, as compared to a corresponding non-transformed wild type non-human organism or a part thereof; and
[101494] (b) growing the non-human organism or a part thereof under conditions which permit the production of hentriacontane or a composition comprising hentriacontane in said non-human organism or in the culture medium surrounding said non-human organism.

[101495] A further embodiment of the present invention relates to a process for the production of hentriacontane, which comprises
[101496] (a) increasing or generating the activity of a sugar transport system permease protein non-targeted in a non-human organism or a part thereof, preferably a microorganism, a plant cell, a plant or a part thereof, as compared to a corresponding non-transformed wild type non-human organism or a part thereof; and
[101497] (b) growing the non-human organism or a part thereof under conditions which permit the production of hentriacontane or a composition comprising hentriacontane in said non-human organism or in the culture medium surrounding said non-human organism.

[101498] A further embodiment of the present invention relates to a process for the production of hentriacontane, which comprises
[101499] (a) increasing or generating the activity of a cyclin D non-targeted in a non-human organism or a part thereof, preferably a microorganism, a plant cell, a plant or a part thereof, as compared to a corresponding non-transformed wild type non-human organism or a part thereof; and
[101500] (b) growing the non-human organism or a part thereof under conditions which permit the production of hentriacontane or a composition comprising hentriacontane in said non-human organism or in the culture medium surrounding said non-human organism.

[101501] A further embodiment of the present invention relates to a process for the production of hentriacontane, which comprises
[101502] (a) increasing or generating the activity of a short-chain alcohol dehydrogenase family non-targeted in a non-human organism or a part thereof, preferably a microorganism, a plant cell, a plant or a part thereof, as compared to a corresponding non-transformed wild type non-human organism or a part thereof; and
[101503] (b) growing the non-human organism or a part thereof under conditions which permit the production of hentriacontane or a composition comprising hentriacontane in said non-human organism or in the culture medium surrounding said non-human organism.

[101504] A further embodiment of the present invention relates to a process for the production of hentriacontane, which comprises
[101505] (a) increasing or generating the activity of a cell division protein non-targeted in a non-human organism or a part thereof, preferably a microorganism, a plant cell, a plant or a part thereof, as compared to a corresponding non-transformed wild type non-human organism or a part thereof; and
[101506] (b) growing the non-human organism or a part thereof under conditions which permit the production of hentriacontane or a composition comprising hentriacontane in said non-human organism or in the culture medium surrounding said non-human organism.

[101507] A further embodiment of the present invention relates to a process for the production of hentriacontane, which comprises
[101508] (a) increasing or generating the activity of a metal-dependent hydrolase non-targeted in a non-human organism or a part thereof, preferably a microorganism, a plant cell, a plant or a part thereof, as compared to a corresponding non-transformed wild type non-human organism or a part thereof; and
[101509] (b) growing the non-human organism or a part thereof under conditions which permit the production of hentriacontane or a composition comprising hentriacontane in said non-human organism or in the culture medium surrounding said non-human organism.

[101510] A further embodiment of the present invention relates to a process for the production of hentriacontane, which comprises
[101511] (a) increasing or generating the activity of a 3-deoxy-7-phosphoheptulonate synthase non-targeted in a non-human organism or a part thereof, preferably a microorganism, a plant cell, a plant or a part thereof, as compared to a corresponding non-transformed wild type non-human organism or a part thereof; and
[101512] (b) growing the non-human organism or a part thereof under conditions which permit the production of hentriacontane or a composition comprising hentriacontane in said non-human organism or in the culture medium surrounding said non-human organism.

[101513] A further embodiment of the present invention relates to a process for the production of hentriacontane, which comprises
[101514] (a) increasing or generating the activity of a YFL019C-protein non-targeted in a non-human organism or a part thereof, preferably a microorganism, a plant cell, a plant or a part thereof, as compared to a corresponding non-transformed wild type non-human organism or a part thereof; and
[101515] (b) growing the non-human organism or a part thereof under conditions which permit the production of hentriacontane or a composition comprising hentriacontane in said non-human organism or in the culture medium surrounding said non-human organism.

[101516] A further embodiment of the present invention relates to a process for the production of hentriacontane, which comprises
[101517] (a) increasing or generating the activity of a dihydroxyacetone kinase non-targeted in a non-human organism or a part thereof, preferably a microorganism, a plant cell, a plant or a part thereof, as compared to a corresponding non-transformed wild type non-human organism or a part thereof; and
[101518] (b) growing the non-human organism or a part thereof under conditions which permit the production of hentriacontane or a composition comprising hentriacontane in said non-human organism or in the culture medium surrounding said non-human organism.

[101519] A further embodiment of the present invention relates to a process for the production of hentriacontane, which comprises
[101520] (a) increasing or generating the activity of a methionine permease non-targeted in a non-human organism or a part thereof, preferably a microorganism, a plant cell, a plant or a part thereof, as compared to a corresponding non-transformed wild type non-human organism or a part thereof; and
[101521] (b) growing the non-human organism or a part thereof under conditions which permit the production of hentriacontane or a composition comprising hentriacontane in said non-human organism or in the culture medium surrounding said non-human organism.

[101522] A further embodiment of the present invention relates to a process for the production of hentriacontane, which comprises
[101523] (a) increasing or generating the activity of a YHL039W-protein non-targeted in a non-human organism or a part thereof, preferably a microorganism, a plant cell, a plant or a part thereof, as compared to a corresponding non-transformed wild type non-human organism or a part thereof; and
[101524] (b) growing the non-human organism or a part thereof under conditions which permit the production of hentriacontane or a composition comprising hentriacontane in said non-human organism or in the culture medium surrounding said non-human organism.

[101525] A further embodiment of the present invention relates to a process for the production of hentriacontane, which comprises
[101526] (a) increasing or generating the activity of a YHR022C-A-protein non-targeted in a non-human organism or a part thereof, preferably a microorganism, a plant cell, a plant or a part thereof, as compared to a corresponding non-transformed wild type non-human organism or a part thereof; and
[101527] (b) growing the non-human organism or a part thereof under conditions which permit the production of hentriacontane or a composition comprising hentriacontane in said non-human organism or in the culture medium surrounding said non-human organism.

[101528] A further embodiment of the present invention relates to a process for the production of hentriacontane, which comprises
[101529] (a) increasing or generating the activity of a YJL127W-A-protein non-targeted in a non-human organism or a part thereof, preferably a microorganism, a plant cell, a plant or a part thereof, as compared to a corresponding non-transformed wild type non-human organism or a part thereof; and
[101530] (b) growing the non-human organism or a part thereof under conditions which permit the production of hentriacontane or a composition comprising hentriacontane in said non-human organism or in the culture medium surrounding said non-human organism.

[101531] A further embodiment of the present invention relates to a process for the production of hentriacontane, which comprises
[101532] (a) increasing or generating the activity of a ylr157c-protein non-targeted in a non-human organism or a part thereof, preferably a microorganism, a plant cell, a plant or a part thereof, as compared to a corresponding non-transformed wild type non-human organism or a part thereof; and
[101533] (b) growing the non-human organism or a part thereof under conditions which permit the production of hentriacontane or a composition comprising hentriacontane in said non-human organism or in the culture medium surrounding said non-human organism.

[101534] A further embodiment of the present invention relates to a process for the production of hentriacontane, which comprises
[101535] (a) increasing or generating the activity of a ynl321w-protein non-targeted in a non-human organism or a part thereof, preferably a microorganism, a plant cell, a plant or a part thereof, as compared to a corresponding non-transformed wild type non-human organism or a part thereof; and
[101536] (b) growing the non-human organism or a part thereof under conditions which permit the production of hentriacontane or a composition comprising hentriacontane in said non-human organism or in the culture medium surrounding said non-human organism.

[101537] A further embodiment of the present invention relates to a process for the production of hentriacontane, which comprises
[101538] (a) increasing or generating the activity of a ypr170c-protein non-targeted in a non-human organism or a part thereof, preferably a microorganism, a plant cell, a plant or a part thereof, as compared to a corresponding non-transformed wild type non-human organism or a part thereof; and
[101539] (b) growing the non-human organism or a part thereof under conditions which permit the production of hentriacontane or a composition comprising hentriacontane in said non-human organism or in the culture medium surrounding said non-human organism.

[101540] A further embodiment of the present invention relates to a process for the production of hentriacontane, which comprises
[101541] (a) increasing or generating the activity of a YPR172W-protein non-targeted in a non-human organism or a part thereof, preferably a microorganism, a plant cell, a plant or a part thereof, as compared to a corresponding non-transformed wild type non-human organism or a part thereof; and
[101542] (b) growing the non-human organism or a part thereof under conditions which permit the production of hentriacontane or a composition comprising hentriacontane in said non-human organism or in the culture medium surrounding said non-human organism.

[101543] A further embodiment of the present invention relates to a process for the production of hentriacontane, which comprises
[101544] (a) increasing or generating the activity of a b3644-protein non-targeted in a non-human organism or a part thereof, preferably a microorganism, a plant cell, a plant or a part thereof, as compared to a corresponding non-transformed wild type non-human organism or a part thereof; and
[101545] (b) growing the non-human organism or a part thereof under conditions which permit the production of hentriacontane or a composition comprising hentriacontane in said non-human organism or in the culture medium surrounding said non-human organism.

[101546] A further embodiment of the present invention relates to a process for the production of hentriacontane, which comprises
[101547] (a) increasing or generating the activity of a polypeptide comprising a polypeptide as depicted in the respective line in column 5 or 8 of Table II, application no. 20, or a homolog or a fragment thereof, a consensus sequence or at least one polypeptide motif as depicted in the respective line in column 8 of Table IV, application no. 20, whereby the respective line disclose in column 7 the fine chemical hentriacontane; or
[101548] increasing or generating the activity of an expression product of one or more nucleic acid molecule(s) comprising a polynucleotide as depicted in the respective line in column 5 or 8 of Table I, application no. 20, preferably the coding region thereof, or a homolog or a fragment thereof, whereby the respective line disclose in column 7 the fine chemical hentriacontane;
[101549] non-targeted in a non-human organism or a part thereof; preferably a microorganism, a plant cell, a plant or a part thereof, as compared to a corresponding non-transformed wild type non-human organism or a part thereof; and
[101550] (b) growing the non-human organism under conditions which permit the production of hentriacontane, or a composition comprising hentriacontane in said non-human organism or in the culture medium surrounding said non-human organism.

[101551] A further embodiment of the present invention relates to a process for the production of hentriacontane, which comprises
[101552] (a) increasing or generating one or more activities selected from the group consisting of pyruvate kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101553] (b) growing the non-human organism or a part thereof under conditions which permit the production of hentriacontane or a composition comprising hentriacontane in said non-human organism or in the culture medium surrounding said non-human organism.

[101554] A further embodiment of the present invention relates to a process for the production of hentriacontane, which comprises
[101555] (a) increasing or generating one or more activities selected from the group consisting of MADS box protein transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101556] (b) growing the non-human organism or a part thereof under conditions which permit the production of hentriacontane or a composition comprising hentriacontane in said non-human organism or in the culture medium surrounding said non-human organism.

[101557] A further embodiment of the present invention relates to a process for the production of hentriacontane, which comprises
[101558] (a) increasing or generating one or more activities selected from the group consisting of At2g45420-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101559] (b) growing the non-human organism or a part thereof under conditions which permit the production of hentriacontane or a composition comprising hentriacontane in said non-human organism or in the culture medium surrounding said non-human organism.

[101560] A further embodiment of the present invention relates to a process for the production of hentriacontane, which comprises
[101561] (a) increasing or generating one or more activities selected from the group consisting of transcription factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101562] (b) growing the non-human organism or a part thereof under conditions which permit the production of hentriacontane or a composition comprising hentriacontane in said non-human organism or in the culture medium surrounding said non-human organism.

[101563] A further embodiment of the present invention relates to a process for the production of hentriacontane, which comprises
[101564] (a) increasing or generating one or more activities selected from the group consisting of At5g43630-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101565] (b) growing the non-human organism or a part thereof under conditions which permit the production of hentriacontane or a composition comprising hentriacontane in said non-human organism or in the culture medium surrounding said non-human organism.

[101566] A further embodiment of the present invention relates to a process for the production of hentriacontane, which comprises
[101567] (a) increasing or generating one or more activities selected from the group consisting of RNA-binding protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101568] (b) growing the non-human organism or a part thereof under conditions which permit the production of hentriacontane or a composition comprising hentriacontane in said non-human organism or in the culture medium surrounding said non-human organism.

[101569] A further embodiment of the present invention relates to a process for the production of hentriacontane, which comprises
[101570] (a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101571] (b) growing the non-human organism or a part thereof under conditions which permit the production of hentriacontane or a composition comprising hentriacontane in said non-human organism or in the culture medium surrounding said non-human organism.

[101572] A further embodiment of the present invention relates to a process for the production of hentriacontane, which comprises
[101573] (a) increasing or generating one or more activities selected from the group consisting of fumarylacetoacetate hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101574] (b) growing the non-human organism or a part thereof under conditions which permit the production of hentriacontane or a composition comprising hentriacontane in said non-human organism or in the culture medium surrounding said non-human organism.

[101575] A further embodiment of the present invention relates to a process for the production of hentriacontane, which comprises
[101576] (a) increasing or generating one or more activities selected from the group consisting of spermidine synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101577] (b) growing the non-human organism or a part thereof under conditions which permit the production of hentriacontane or a composition comprising hentriacontane in said non-human organism or in the culture medium surrounding said non-human organism.

[101578] A further embodiment of the present invention relates to a process for the production of hentriacontane, which comprises
[101579] (a) increasing or generating one or more activities selected from the group consisting of serine protease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101580] (b) growing the non-human organism or a part thereof under conditions which permit the production of hentriacontane or a composition comprising hentriacontane in said non-human organism or in the culture medium surrounding said non-human organism.

[101581] A further embodiment of the present invention relates to a process for the production of hentriacontane, which comprises
[101582] (a) increasing or generating one or more activities selected from the group consisting of peptide chain release factor in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101583] (b) growing the non-human organism or a part thereof under conditions which permit the production of hentriacontane or a composition comprising hentriacontane in said non-human organism or in the culture medium surrounding said non-human organism.

[101584] A further embodiment of the present invention relates to a process for the production of hentriacontane, which comprises
[101585] (a) increasing or generating one or more activities selected from the group consisting of mechanosensitive ion channel in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101586] (b) growing the non-human organism or a part thereof under conditions which permit the production of hentriacontane or a composition comprising hentriacontane in said non-human organism or in the culture medium surrounding said non-human organism.

[101587] A further embodiment of the present invention relates to a process for the production of hentriacontane, which comprises
[101588] (a) increasing or generating one or more activities selected from the group consisting of pyrimidine-specific ribonucleoside hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101589] (b) growing the non-human organism or a part thereof under conditions which permit the production of hentriacontane or a composition comprising hentriacontane in said non-human organism or in the culture medium surrounding said non-human organism.

[101590] A further embodiment of the present invention relates to a process for the production of hentriacontane, which comprises
[101591] (a) increasing or generating one or more activities selected from the group consisting of inner membrane protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101592] (b) growing the non-human organism or a part thereof under conditions which permit the production of hentriacontane or a composition comprising hentriacontane in said non-human organism or in the culture medium surrounding said non-human organism.

[101593] A further embodiment of the present invention relates to a process for the production of hentriacontane, which comprises
[101594] (a) increasing or generating one or more activities selected from the group consisting of b0833-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101595] (b) growing the non-human organism or a part thereof under conditions which permit the production of hentriacontane or a composition comprising hentriacontane in said non-human organism or in the culture medium surrounding said non-human organism.

[101596] A further embodiment of the present invention relates to a process for the production of hentriacontane, which comprises
[101597] (a) increasing or generating one or more activities selected from the group consisting of S-adenosyl-L-methionine-dependent methyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101598] (b) growing the non-human organism or a part thereof under conditions which permit the production of hentriacontane or a composition comprising hentriacontane in said non-human organism or in the culture medium surrounding said non-human organism.

[101599] A further embodiment of the present invention relates to a process for the production of hentriacontane, which comprises
[101600] (a) increasing or generating one or more activities selected from the group consisting of b1259-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101601] (b) growing the non-human organism or a part thereof under conditions which permit the production of hentriacontane or a composition comprising hentriacontane in said non-human organism or in the culture medium surrounding said non-human organism.

[101602] A further embodiment of the present invention relates to a process for the production of hentriacontane, which comprises
[101603] (a) increasing or generating one or more activities selected from the group consisting of orotidine-5′-phosphate decarboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101604] (b) growing the non-human organism or a part thereof under conditions which permit the production of hentriacontane or a composition comprising hentriacontane in said non-human organism or in the culture medium surrounding said non-human organism.

[101605] A further embodiment of the present invention relates to a process for the production of hentriacontane, which comprises
[101606] (a) increasing or generating one or more activities selected from the group consisting of ATP-dependent RNA helicase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101607] (b) growing the non-human organism or a part thereof under conditions which permit the production of hentriacontane or a composition comprising hentriacontane in said non-human organism or in the culture medium surrounding said non-human organism.

[101608] A further embodiment of the present invention relates to a process for the production of hentriacontane, which comprises
[101609] (a) increasing or generating one or more activities selected from the group consisting of b1445-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101610] (b) growing the non-human organism or a part thereof under conditions which permit the production of hentriacontane or a composition comprising hentriacontane in said non-human organism or in the culture medium surrounding said non-human organism.

[101611] A further embodiment of the present invention relates to a process for the production of hentriacontane, which comprises
[101612] (a) increasing or generating one or more activities selected from the group consisting of acid shock protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101613] (b) growing the non-human organism or a part thereof under conditions which permit the production of hentriacontane or a composition comprising hentriacontane in said non-human organism or in the culture medium surrounding said non-human organism.

[101614] A further embodiment of the present invention relates to a process for the production of hentriacontane, which comprises
[101615] (a) increasing or generating one or more activities selected from the group consisting of electron transport complex protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101616] (b) growing the non-human organism or a part thereof under conditions which permit the production of hentriacontane or a composition comprising hentriacontane in said non-human organism or in the culture medium surrounding said non-human organism.

[101617] A further embodiment of the present invention relates to a process for the production of hentriacontane, which comprises
[101618] (a) increasing or generating one or more activities selected from the group consisting of transcriptional regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101619] (b) growing the non-human organism or a part thereof under conditions which permit the production of hentriacontane or a composition comprising hentriacontane in said non-human organism or in the culture medium surrounding said non-human organism.

[101620] A further embodiment of the present invention relates to a process for the production of hentriacontane, which comprises
[101621] (a) increasing or generating one or more activities selected from the group consisting of b1670-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101622] (b) growing the non-human organism or a part thereof under conditions which permit the production of hentriacontane or a composition comprising hentriacontane in said non-human organism or in the culture medium surrounding said non-human organism.

[101623] A further embodiment of the present invention relates to a process for the production of hentriacontane, which comprises
[101624] (a) increasing or generating one or more activities selected from the group consisting of phospho-2-dehydro-3-deoxyheptonate aldolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101625] (b) growing the non-human organism or a part thereof under conditions which permit the production of hentriacontane or a composition comprising hentriacontane in said non-human organism or in the culture medium surrounding said non-human organism.

[101626] A further embodiment of the present invention relates to a process for the production of hentriacontane, which comprises
[101627] (a) increasing or generating one or more activities selected from the group consisting of b1930-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101628] (b) growing the non-human organism or a part thereof under conditions which permit the production of hentriacontane or a composition comprising hentriacontane in said non-human organism or in the culture medium surrounding said non-human organism.

[101629] A further embodiment of the present invention relates to a process for the production of hentriacontane, which comprises
[101630] (a) increasing or generating one or more activities selected from the group consisting of O-antigen chain length determinant in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101631] (b) growing the non-human organism or a part thereof under conditions which permit the production of hentriacontane or a composition comprising hentriacontane in said non-human organism or in the culture medium surrounding said non-human organism.

[101632] A further embodiment of the present invention relates to a process for the production of hentriacontane, which comprises
[101633] (a) increasing or generating one or more activities selected from the group consisting of GDP-mannose 4,6-dehydratase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101634] (b) growing the non-human organism or a part thereof under conditions which permit the production of hentriacontane or a composition comprising hentriacontane in said non-human organism or in the culture medium surrounding said non-human organism.

[101635] A further embodiment of the present invention relates to a process for the production of hentriacontane, which comprises
[101636] (a) increasing or generating one or more activities selected from the group consisting of assembly protein for periplasmic nitrate reductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101637] (b) growing the non-human organism or a part thereof under conditions which permit the production of hentriacontane or a composition comprising hentriacontane in said non-human organism or in the culture medium surrounding said non-human organism.

[101638] A further embodiment of the present invention relates to a process for the production of hentriacontane, which comprises
[101639] (a) increasing or generating one or more activities selected from the group consisting of ion-transport protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101640] (b) growing the non-human organism or a part thereof under conditions which permit the production of hentriacontane or a composition comprising hentriacontane in said non-human organism or in the culture medium surrounding said non-human organism.

[101641] A further embodiment of the present invention relates to a process for the production of hentriacontane, which comprises
[101642] (a) increasing or generating one or more activities selected from the group consisting of b2399-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101643] (b) growing the non-human organism or a part thereof under conditions which permit the production of hentriacontane or a composition comprising hentriacontane in said non-human organism or in the culture medium surrounding said non-human organism.

[101644] A further embodiment of the present invention relates to a process for the production of hentriacontane, which comprises
[101645] (a) increasing or generating one or more activities selected from the group consisting of cysteine synthase A in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101646] (b) growing the non-human organism or a part thereof under conditions which permit the production of hentriacontane or a composition comprising hentriacontane in said non-human organism or in the culture medium surrounding said non-human organism.

[101647] A further embodiment of the present invention relates to a process for the production of hentriacontane, which comprises
[101648] (a) increasing or generating one or more activities selected from the group consisting of ethanolamine utilization protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101649] (b) growing the non-human organism or a part thereof under conditions which permit the production of hentriacontane or a composition comprising hentriacontane in said non-human organism or in the culture medium surrounding said non-human organism.

[101650] A further embodiment of the present invention relates to a process for the production of hentriacontane, which comprises
[101651] (a) increasing or generating one or more activities selected from the group consisting of sigma-54 dependent response regulator in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101652] (b) growing the non-human organism or a part thereof under conditions which permit the production of hentriacontane or a composition comprising hentriacontane in said non-human organism or in the culture medium surrounding said non-human organism.

[101653] A further embodiment of the present invention relates to a process for the production of hentriacontane, which comprises
[101654] (a) increasing or generating one or more activities selected from the group consisting of b2653-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101655] (b) growing the non-human organism or a part thereof under conditions which permit the production of hentriacontane or a composition comprising hentriacontane in said non-human organism or in the culture medium surrounding said non-human organism.

[101656] A further embodiment of the present invention relates to a process for the production of hentriacontane, which comprises
[101657] (a) increasing or generating one or more activities selected from the group consisting of phosphoadenosine phosphosulfate reductase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101658] (b) growing the non-human organism or a part thereof under conditions which permit the production of hentriacontane or a composition comprising hentriacontane in said non-human organism or in the culture medium surrounding said non-human organism.

[101659] A further embodiment of the present invention relates to a process for the production of hentriacontane, which comprises
[101660] (a) increasing or generating one or more activities selected from the group consisting of amino-acid acetyltransferase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101661] (b) growing the non-human organism or a part thereof under conditions which permit the production of hentriacontane or a composition comprising hentriacontane in said non-human organism or in the culture medium surrounding said non-human organism.

[101662] A further embodiment of the present invention relates to a process for the production of hentriacontane, which comprises
[101663] (a) increasing or generating one or more activities selected from the group consisting of exonuclease subunit in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101664] (b) growing the non-human organism or a part thereof under conditions which permit the production of hentriacontane or a composition comprising hentriacontane in said non-human organism or in the culture medium surrounding said non-human organism.

[101665] A further embodiment of the present invention relates to a process for the production of hentriacontane, which comprises
[101666] (a) increasing or generating one or more activities selected from the group consisting of L-asparaginase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101667] (b) growing the non-human organism or a part thereof under conditions which permit the production of hentriacontane or a composition comprising hentriacontane in said non-human organism or in the culture medium surrounding said non-human organism.

[101668] A further embodiment of the present invention relates to a process for the production of hentriacontane, which comprises
[101669] (a) increasing or generating one or more activities selected from the group consisting of ornithine decarboxylase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101670] (b) growing the non-human organism or a part thereof under conditions which permit the production of hentriacontane or a composition comprising hentriacontane in said non-human organism or in the culture medium surrounding said non-human organism.

[101671] A further embodiment of the present invention relates to a process for the production of hentriacontane, which comprises
[101672] (a) increasing or generating one or more activities selected from the group consisting of phosphoglycolate phosphatase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101673] (b) growing the non-human organism or a part thereof under conditions which permit the production of hentriacontane or a composition comprising hentriacontane in said non-human organism or in the culture medium surrounding said non-human organism.

[101674] A further embodiment of the present invention relates to a process for the production of hentriacontane, which comprises
[101675] (a) increasing or generating one or more activities selected from the group consisting of sugar transport system permease protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101676] (b) growing the non-human organism or a part thereof under conditions which permit the production of hentriacontane or a composition comprising hentriacontane in said non-human organism or in the culture medium surrounding said non-human organism.

[101677] A further embodiment of the present invention relates to a process for the production of hentriacontane, which comprises
[101678] (a) increasing or generating one or more activities selected from the group consisting of cyclin D in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101679] (b) growing the non-human organism or a part thereof under conditions which permit the production of hentriacontane or a composition comprising hentriacontane in said non-human organism or in the culture medium surrounding said non-human organism.

[101680] A further embodiment of the present invention relates to a process for the production of hentriacontane, which comprises
[101681] (a) increasing or generating one or more activities selected from the group consisting of short-chain alcohol dehydrogenase family in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101682] (b) growing the non-human organism or a part thereof under conditions which permit the production of hentriacontane or a composition comprising hentriacontane in said non-human organism or in the culture medium surrounding said non-human organism.

[101683] A further embodiment of the present invention relates to a process for the production of hentriacontane, which comprises
[101684] (a) increasing or generating one or more activities selected from the group consisting of cell division protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101685] (b) growing the non-human organism or a part thereof under conditions which permit the production of hentriacontane or a composition comprising hentriacontane in said non-human organism or in the culture medium surrounding said non-human organism.

[101686] A further embodiment of the present invention relates to a process for the production of hentriacontane, which comprises
[101687] (a) increasing or generating one or more activities selected from the group consisting of metal-dependent hydrolase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101688] (b) growing the non-human organism or a part thereof under conditions which permit the production of hentriacontane or a composition comprising hentriacontane in said non-human organism or in the culture medium surrounding said non-human organism.

[101689] A further embodiment of the present invention relates to a process for the production of hentriacontane, which comprises
[101690] (a) increasing or generating one or more activities selected from the group consisting of 3-deoxy-7-phosphoheptulonate synthase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101691] (b) growing the non-human organism or a part thereof under conditions which permit the production of hentriacontane or a composition comprising hentriacontane in said non-human organism or in the culture medium surrounding said non-human organism.

[101692] A further embodiment of the present invention relates to a process for the production of hentriacontane, which comprises
[101693] (a) increasing or generating one or more activities selected from the group consisting of YFL019C-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101694] (b) growing the non-human organism or a part thereof under conditions which permit the production of hentriacontane or a composition comprising hentriacontane in said non-human organism or in the culture medium surrounding said non-human organism.

[101695] A further embodiment of the present invention relates to a process for the production of hentriacontane, which comprises
[101696] (a) increasing or generating one or more activities selected from the group consisting of dihydroxyacetone kinase in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101697] (b) growing the non-human organism or a part thereof under conditions which permit the production of hentriacontane or a composition comprising hentriacontane in said non-human organism or in the culture medium surrounding said non-human organism.

[101698] A further embodiment of the present invention relates to a process for the production of hentriacontane, which comprises
[101699] (a) increasing or generating one or more activities selected from the group consisting of methionine permease in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101700] (b) growing the non-human organism or a part thereof under conditions which permit the production of hentriacontane or a composition comprising hentriacontane in said non-human organism or in the culture medium surrounding said non-human organism.

[101701] A further embodiment of the present invention relates to a process for the production of hentriacontane, which comprises
[101702] (a) increasing or generating one or more activities selected from the group consisting of YHL039W-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101703] (b) growing the non-human organism or a part thereof under conditions which permit the production of hentriacontane or a composition comprising hentriacontane in said non-human organism or in the culture medium surrounding said non-human organism.

[101704] A further embodiment of the present invention relates to a process for the production of hentriacontane, which comprises
[101705] (a) increasing or generating one or more activities selected from the group consisting of YHR022C-A-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101706] (b) growing the non-human organism or a part thereof under conditions which permit the production of hentriacontane or a composition comprising hentriacontane in said non-human organism or in the culture medium surrounding said non-human organism.

[101707] A further embodiment of the present invention relates to a process for the production of hentriacontane, which comprises
[101708] (a) increasing or generating one or more activities selected from the group consisting of YJL127W-A-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101709] (b) growing the non-human organism or a part thereof under conditions which permit the production of hentriacontane or a composition comprising hentriacontane in said non-human organism or in the culture medium surrounding said non-human organism.

[101710] A further embodiment of the present invention relates to a process for the production of hentriacontane, which comprises
[101711] (a) increasing or generating one or more activities selected from the group consisting of ylr157c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101712] (b) growing the non-human organism or a part thereof under conditions which permit the production of hentriacontane or a composition comprising hentriacontane in said non-human organism or in the culture medium surrounding said non-human organism.

[101713] A further embodiment of the present invention relates to a process for the production of hentriacontane, which comprises
[101714] (a) increasing or generating one or more activities selected from the group consisting of ynl321w-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101715] (b) growing the non-human organism or a part thereof under conditions which permit the production of hentriacontane or a composition comprising hentriacontane in said non-human organism or in the culture medium surrounding said non-human organism.

[101716] A further embodiment of the present invention relates to a process for the production of hentriacontane, which comprises
[101717] (a) increasing or generating one or more activities selected from the group consisting of ypr170c-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101718] (b) growing the non-human organism or a part thereof under conditions which permit the production of hentriacontane or a composition comprising hentriacontane in said non-human organism or in the culture medium surrounding said non-human organism.

[101719] A further embodiment of the present invention relates to a process for the production of hentriacontane, which comprises
[101720] (a) increasing or generating one or more activities selected from the group consisting of YPR172W-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101721] (b) growing the non-human organism or a part thereof under conditions which permit the production of hentriacontane or a composition comprising hentriacontane in said non-human organism or in the culture medium surrounding said non-human organism.

[101722] A further embodiment of the present invention relates to a process for the production of hentriacontane, which comprises
[101723] (a) increasing or generating one or more activities selected from the group consisting of b3644-protein in an organelle, preferably in plastids or mitochondria, especially in plastids, of a non-human organism or a part thereof, preferably a microorganism, a plant cell, a plant or a part thereof, as compared to a corresponding non-transformed wild type non-human organism or a part thereof; and
[101724] (b) growing the non-human organism or a part thereof under conditions which permit the production of hentriacontane or a composition comprising hentriacontane in said non-human organism or in the culture medium surrounding said non-human organism.

[101725] A further embodiment of the present invention relates to a process for the production of hentriacontane, which comprises
[101726] (a1) increasing or generating the activity of a polypeptide comprising a polypeptide as depicted in the respective line in column 5 or 8 of Table II, application no. 20, or a homolog or fragment thereof, a consensus sequence or at least one polypeptide motif as depicted in column 8 of Table IV, application no. 20, whereby the respective line disclose in column 7 the fine chemical hentriacontane; or
[101727] increasing or generating the activity of an expression product of one or more nucleic acid molecule(s) comprising a polynucleotide as depicted in the respective line in column 5 or 8 of Table I, application no. 20, preferably the coding region thereof, or a homolog or a fragment thereof, whereby the respective line disclose in column 7 the fine chemical hentriacontane;
[101728] in an organelle, preferably in plastids or mitochondria, especially in plastids, in a non-human organism or a part thereof; preferably a microorganism, a plant cell, a plant or a part thereof, as compared to a corresponding non-transformed wild type non-human organism or a part thereof; or
[101729] (a2) increasing or generating the activity of a polypeptide comprising a polypeptide as depicted in the respective line in column 5 or 8 of Table II, application no. 20, or a homolog or a fragment thereof, a consensus sequence or at least one polypeptide motif as depicted in the respective line in column 8 of Table IV, application no. 20, which is joined to a transit peptide, whereby the respective line disclose in column 7 the fine chemical hentriacontane; or
[101730] increasing or generating the activity of an expression product of one or more nucleic acid molecule(s) comprising a polynucleotide as depicted in the respective line in column 5 or 8 of Table I, application no. 20, preferably the coding region thereof, or a homolog or a fragment thereof, which is joined to a nucleic acid sequence encoding an organelle localization sequence, preferably a plastid or a mitochondrion localization sequence, especially a plastid localization sequence, whereby the respective line disclose in column 7 the fine chemical hentriacontane;
[101731] in a non-human organism or a part thereof; preferably a microorganism, a plant cell, a plant or a part thereof, as compared to a corresponding non-transformed wild type non-human organism or a part thereof; or
[101732] (a3) increasing or generating the activity of a polypeptide comprising a polypeptide as depicted
[101733] in the respective line in column 5 or 8 of Table II, application no. 20, or a homolog or a fragment thereof, a consensus sequence or at least one polypeptide motif as depicted in the respective line in column 8 of Table IV, application no. 20, whereby the respective line disclose in column 7 the fine chemical hentriacontane; or increasing or generating the activity of an expression product of one or more nucleic acid molecule(s) comprising a polynucleotide as depicted in the respective line in column 5 or 8 of Table I, application no. 20, preferably the coding region thereof, or a homolog or a fragment thereof, whereby the respective line disclose in column 7 the fine chemical hentriacontane;
[101734] in an organelle, preferably in plastids or mitochondria, especially in plastids, in a non-human organism or a part thereof; preferably a microorganism, a plant cell, a plant or a part thereof, through transformation of the organelle, as compared to a corresponding non-transformed wild type non-human organism or a part thereof;
[101735] and
[101736] (b) growing the non-human organism under conditions which permit the production of hentriacontane, or a composition comprising hentriacontane in said non-human organism or in the culture medium surrounding said non-human organism.

[101737] Accordingly, the present invention relates to a process for the production of hentriacontane, which comprises
[101738] (a) increasing or generating the activity of a pyruvate kinase in the cytosol of a cell of a non-human organism or a part thereof, preferably a microorganism, a plant cell, a plant or a part thereof, as compared to a corresponding non-transformed wild type non-human organism or a part thereof; and
[101739] (b) growing the non-human organism or a part thereof under conditions which permit the production of hentriacontane or a composition comprising hentriacontane in said non-human organism or in the culture medium surrounding said non-human organism.

[101740] Accordingly, the present invention relates to a process for the production of hentriacontane, which comprises
[101741] (a) increasing or generating the activity of a MADS box protein transcription factor in the cytosol of a cell of a non-human organism or a part thereof, preferably a microorganism, a plant cell, a plant or a part thereof, as compared to a corresponding non-transformed wild type non-human organism or a part thereof; and
[101742] (b) growing the non-human organism or a part thereof under conditions which permit the production of hentriacontane or a composition comprising hentriacontane in said non-human organism or in the culture medium surrounding said non-human organism.

[101743] Accordingly, the present invention relates to a process for the production of hentriacontane, which comprises
[101744] (a) increasing or generating the activity of a At2g45420-protein in the cytosol of a cell of a non-human organism or a part thereof, preferably a microorganism, a plant cell, a plant or a part thereof, as compared to a corresponding non-transformed wild type non-human organism or a part thereof; and
[101745] (b) growing the non-human organism or a part thereof under conditions which permit the production of hentriacontane or a composition comprising hentriacontane in said non-human organism or in the culture medium surrounding said non-human organism.

[101746] Accordingly, the present invention relates to a process for the production of hentriacontane, which comprises
[101747] (a) increasing or generating the activity of a transcription factor in the cytosol of a cell of a non-human organism or a part thereof, preferably a microorganism, a plant cell, a plant or a part thereof, as compared to a corresponding non-transformed wild type non-human organism or a part thereof; and
[101748] (b) growing the non-human organism or a part thereof under conditions which permit the production of hentriacontane or a composition comprising hentriacontane in said non-human organism or in the culture medium surrounding said non-human organism.

[101749] Accordingly, the present invention relates to a process for the production of hentriacontane, which comprises
[101750] (a) increasing or generating the activity of a At5g43630-protein in the cytosol of a cell of a non-human organism or a part thereof, preferably a microorganism, a plant cell, a plant or a part thereof, as compared to a corresponding non-transformed wild type non-human organism or a part thereof; and
[101751] (b) growing the non-human organism or a part thereof under conditions which permit the production of hentriacontane or a composition comprising hentriacontane in said non-human organism or in the culture medium surrounding said non-human organism.

[101752] Accordingly, the present invention relates to a process for the production of hentriacontane, which comprises
[101753] (a) increasing or generating the activity of a RNA-binding protein in the cytosol of a cell of a non-human organism or a part thereof, preferably a microorganism, a plant cell, a plant or a part thereof, as compared to a corresponding non-transformed wild type non-human organism or a part thereof; and
[101754] (b) growing the non-human organism or a part thereof under conditions which permit the production of hentriacontane or a composition comprising hentriacontane in said non-human organism or in the culture medium surrounding said non-human organism.

[101755] Accordingly, the present invention relates to a process for the production of hentriacontane, which comprises
[101756] (a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
[101757] (b) growing the non-human organism or a part thereof under conditions which permit the production of hentriacontane or a composition comprising hentriacontane in said non-human organism or in the culture medium surrounding said non-human organism.

[101758] Accordingly, the present invention relates to a process for the production of hentriacontane, which comprises
[101759] (a) increasing or generating the activity of a fumarylacetoacetate hydrolase in the cytosol of a cell of a non-human organism or a part thereof, preferably a microorganism, a plant cell, a plant or a part thereof, as compared to a corresponding non-transformed wild type non-human organism or a part thereof; and
[101760] (b) growing the non-human organism or a part thereof under conditions which permit the production of hentriacontane or a composition comprising hentriacontane in said non-human organism or in the culture medium surrounding said non-human organism.

[101761] Accordingly, the present invention relates to a process for the production of hentriacontane, which comprises
[101762] (a) increasing or generating the activity of a spermidine synthase in the cytosol of a cell of a non-human organism or a part thereof, preferably a microorganism, a plant cell, a plant or a part thereof, as compared to a corresponding non-transformed wild type non-human organism or a part thereof; and
[101763] (b) growing the non-human organism or a part thereof under conditions which permit the production of hentriacontane or a composition comprising hentriacontane in said non-human organism or in the culture medium surrounding said non-human organism.

[101764] Accordingly, the present invention relates to a process for the production of hentriacontane, which comprises
[101765] (a) increasing or generating the activity of a serine protease in the cytosol of a cell of a non-human organism or a part thereof, preferably a microorganism, a plant cell, a plant or a part thereof, as compared to a corresponding non-transformed wild type non-human organism or a part thereof; and
[101766] (b) growing the non-human organism or a part thereof under conditions which permit the production of hentriacontane or a composition comprising hentriacontane in said non-human organism or in the culture medium surrounding said non-human organism.

[101767] Accordingly, the present invention relates to a process for the production of hentriacontane, which comprises
[101768] (a) increasing or generating the activity of a peptide chain release factor in the cytosol of a cell of a non-human organism or a part thereof, preferably a microorganism, a plant cell, a plant or a part thereof, as compared to a corresponding non-transformed wild type non-human organism or a part thereof; and
[101769] (b) growing the non-human organism or a part thereof under conditions which permit the production of hentriacontane or a composition comprising hentriacontane in said non-human organism or in the culture medium surrounding said non-human organism.

[101770] Accordingly, the present invention relates to a process for the production of hentriacontane, which comprises
[101771] (a) increasing or generating the activity of a mechanosensitive ion channel in the cytosol of a cell of a non-human organism or a part thereof, preferably a microorganism, a plant cell, a plant or a part thereof, as compared to a corresponding non-transformed wild type non-human organism or a part thereof; and
[101772] (b) growing the non-human organism or a part thereof under conditions which permit the production of hentriacontane or a composition comprising hentriacontane in said non-human organism or in the culture medium surrounding said non-human organism.

[101773] Accordingly, the present invention relates to a process for the production of hentriacontane, which comprises
[101774] (a) increasing or generating the activity of a pyrimidine-specific ribonucleoside hydrolase in the cytosol of a cell of a non-human organism or a part thereof, preferably a microorganism, a plant cell, a plant or a part thereof, as compared to a corresponding non-transformed wild type non-human organism or a part thereof; and
[101775] (b) growing the non-human organism or a part thereof under conditions which permit the production of hentriacontane or a composition comprising hentriacontane in said non-human organism or in the culture medium surrounding said non-human organism.

[101776] Accordingly, the present invention relates to a process for the production of hentriacontane, which comprises
[101777] (a) increasing or generating the activity of a inner membrane protein in the cytosol of a cell of a non-human organism or a part thereof, preferably a microorganism, a plant cell, a plant or a part thereof, as compared to a corresponding non-transformed wild type non-human organism or a part thereof; and
[101778] (b) growing the non-human organism or a part thereof under conditions which permit the production of hentriacontane or a composition comprising hentriacontane in said non-human organism or in the culture medium surrounding said non-human organism.

[101779] Accordingly, the present invention relates to a process for the production of hentriacontane, which comprises
[101780] (a) increasing or generating the activity of a b0833-protein in the cytosol of a cell of a non-human organism or a part thereof, preferably a microorganism, a plant cell, a plant or a part thereof, as compared to a corresponding non-transformed wild type non-human organism or a part thereof; and
[101781] (b) growing the non-human organism or a part thereof under conditions which permit the production of hentriacontane or a composition comprising hentriacontane in said non-human organism or in the culture medium surrounding said non-human organism.

[101782] Accordingly, the present invention relates to a process for the production of hentriacontane, which comprises
[101783] (a) increasing or generating the activity of a S-adenosyl-L-methionine-dependent methyltransferase in the cytosol of a cell of a non-human organism or a part thereof, preferably a microorganism, a plant cell, a plant or a part thereof, as compared to a corresponding non-transformed wild type non-human organism or a part thereof; and
[101784] (b) growing the non-human organism or a part thereof under conditions which permit the production of hentriacontane or a composition comprising hentriacontane in said non-human organism or in the culture medium surrounding said non-human organism.

[101785] Accordingly, the present invention relates to a process for the production of hentriacontane, which comprises
[101786] (a) increasing or generating the activity of a b1259-protein in the cytosol of a cell of a non-human organism or a part thereof, preferably a microorganism, a plant cell, a plant or a part thereof, as compared to a corresponding non-transformed wild type non-human organism or a part thereof; and
[101787] (b) growing the non-human organism or a part thereof under conditions which permit the production of hentriacontane or a composition comprising hentriacontane in said non-human organism or in the culture medium surrounding said non-human organism.

[101788] Accordingly, the present invention relates to a process for the production of hentriacontane, which comprises
[101789] (a) increasing or generating the activity of a orotidine-5′-phosphate decarboxylase in the cytosol of a cell of a non-human organism or a part thereof, preferably a microorganism, a plant cell, a plant or a part thereof, as compared to a corresponding non-transformed wild type non-human organism or a part thereof; and
[101790] (b) growing the non-human organism or a part thereof under conditions which permit the production of hentriacontane or a composition comprising hentriacontane in said non-human organism or in the culture medium surrounding said non-human organism.

[101791] Accordingly, the present invention relates to a process for the production of hentriacontane, which comprises
[101792] (a) increasing or generating the activity of a ATP-dependent RNA helicase in the cytosol of a cell of a non-human organism or a part thereof, preferably a microorganism, a plant cell, a plant or a part thereof, as compared to a corresponding non-transformed wild type non-human organism or a part thereof; and
[101793] (b) growing the non-human organism or a part thereof under conditions which permit the production of hentriacontane or a composition comprising hentriacontane in said non-human organism or in the culture medium surrounding said non-human organism.

[101794] Accordingly, the present invention relates to a process for the production of hentriacontane, which comprises
[101795] (a) increasing or generating the activity of a b1445-protein in the cytosol of a cell of a non-human organism or a part thereof, preferably a microorganism, a plant cell, a plant or a part thereof, as compared to a corresponding non-transformed wild type non-human organism or a part thereof; and
[101796] (b) growing the non-human organism or a part thereof under conditions which permit the production of hentriacontane or a composition comprising hentriacontane in said non-human organism or in the culture medium surrounding said non-human organism.

[101797] Accordingly, the present invention relates to a process for the production of hentriacontane, which comprises
[101798] (a) increasing or generating the activity of a acid shock protein in the cytosol of a cell of a non-human organism or a part thereof, preferably a microorganism, a plant cell, a plant or a part thereof, as compared to a corresponding non-transformed wild type non-human organism or a part thereof; and
[101799] (b) growing the non-human organism or a part thereof under conditions which permit the production of hentriacontane or a composition comprising hentriacontane in said non-human organism or in the culture medium surrounding said non-human organism.

[101800] Accordingly, the present invention relates to a process for the production of hentriacontane, which comprises
[101801] (a) increasing or generating the activity of a electron transport complex protein in the cytosol of a cell of a non-human organism or a part thereof, preferably a microorganism, a plant cell, a plant or a part thereof, as compared to a corresponding non-transformed wild type non-human organism or a part thereof; and
[101802] (b) growing the non-human organism or a part thereof under conditions which permit the production of hentriacontane or a composition comprising hentriacontane in said non-human organism or in the culture medium surrounding said non-human organism.

[101803] Accordingly, the present invention relates to a process for the production of hentriacontane, which comprises
[101804] (a) increasing or generating the activity of a transcriptional regulator in the cytosol of a cell of a non-human organism or a part thereof, preferably a microorganism, a plant cell, a plant or a part thereof, as compared to a corresponding non-transformed wild type non-human organism or a part thereof; and
[101805] (b) growing the non-human organism or a part thereof under conditions which permit the production of hentriacontane or a composition comprising hentriacontane in said non-human organism or in the culture medium surrounding said non-human organism.

[101806] Accordingly, the present invention relates to a process for the production of hentriacontane, which comprises
[101807] (a) increasing or generating the activity of a b1670-protein in the cytosol of a cell of a non-human organism or a part thereof, preferably a microorganism, a plant cell, a plant or a part thereof, as compared to a corresponding non-transformed wild type non-human organism or a part thereof; and
[101808] (b) growing the non-human organism or a part thereof under conditions which permit the production of hentriacontane or a composition comprising hentriacontane in said non-human organism or in the culture medium surrounding said non-human organism.

[101809] Accordingly, the present invention relates to a process for the production of hentriacontane, which comprises
[101810] (a) increasing or generating the activity of a phospho-2-dehydro-3-deoxyheptonate aldolase in the cytosol of a cell of a non-human organism or a part thereof, preferably a microorganism, a plant cell, a plant or a part thereof, as compared to a corresponding non-transformed wild type non-human organism or a part thereof; and
[101811] (b) growing the non-human organism or a part thereof under conditions which permit the production of hentriacontane or a composition comprising hentriacontane in said non-human organism or in the culture medium surrounding said non-human organism.

[101812] Accordingly, the present invention relates to a process for the production of hentriacontane, which comprises
[101813] (a) increasing or generating the activity of a b1930-protein in the cytosol of a cell of a non-human organism or a part thereof, preferably a microorganism, a plant cell, a plant or a part thereof, as compared to a corresponding non-transformed wild type non-human organism or a part thereof; and
[101814] (b) growing the non-human organism or a part thereof under conditions which permit the production of hentriacontane or a composition comprising hentriacontane in said non-human organism or in the culture medium surrounding said non-human organism.

[101815] Accordingly, the present invention relates to a process for the production of hentriacontane, which comprises
[101816] (a) increasing or generating the activity of a O-antigen chain length determinant in the cytosol of a cell of a non-human organism or a part thereof, preferably a microorganism, a plant cell, a plant or a part thereof, as compared to a corresponding non-transformed wild type non-human organism or a part thereof; and
[101817] (b) growing the non-human organism or a part thereof under conditions which permit the production of hentriacontane or a composition comprising hentriacontane in said non-human organism or in the culture medium surrounding said non-human organism.

[101818] Accordingly, the present invention relates to a process for the production of hentriacontane, which comprises
[101819] (a) increasing or generating the activity of a GDP-mannose 4,6-dehydratase in the cytosol of a cell of a non-human organism or a part thereof, preferably a microorganism, a plant cell, a plant or a part thereof, as compared to a corresponding non-transformed wild type non-human organism or a part thereof; and
[101820] (b) growing the non-human organism or a part thereof under conditions which permit the production of hentriacontane or a composition comprising hentriacontane in said non-human organism or in the culture medium surrounding said non-human organism.

[101821] Accordingly, the present invention relates to a process for the production of hentriacontane, which comprises
[101822] (a) increasing or generating the activity of a assembly protein for periplasmic nitrate reductase in the cytosol of a cell of a non-human organism or a part thereof, preferably a microorganism, a plant cell, a plant or a part thereof, as compared to a corresponding non-transformed wild type non-human organism or a part thereof; and
[101823] (b) growing the non-human organism or a part thereof under conditions which permit the production of hentriacontane or a composition comprising hentriacontane in said non-human organism or in the culture medium surrounding said non-human organism.

[101824] Accordingly, the present invention relates to a process for the production of hentriacontane, which comprises
[101825] (a) increasing or generating the activity of a ion-transport protein in the cytosol of a cell of a non-human organism or a part thereof, preferably a microorganism, a plant cell, a plant or a part thereof, as compared to a corresponding non-transformed wild type non-human organism or a part thereof; and
[101826] (b) growing the non-human organism or a part thereof under conditions which permit the production of hentriacontane or a composition comprising hentriacontane in said non-human organism or in the culture medium surrounding said non-human organism.

[101827] Accordingly, the present invention relates to a process for the production of hentriacontane, which comprises
[101828] (a) increasing or generating the activity of a b2399-protein in the cytosol of a cell of a non-human organism or a part thereof, preferably a microorganism, a plant cell, a plant or a part thereof, as compared to a corresponding non-transformed wild type non-human organism or a part thereof; and
[101829] (b) growing the non-human organism or a part thereof under conditions which permit the production of hentriacontane or a composition comprising hentriacontane in said non-human organism or in the culture medium surrounding said non-human organism.

[101830] Accordingly, the present invention relates to a process for the production of hentriacontane, which comprises
[101831] (a) increasing or generating the activity of a cysteine synthase A in the cytosol of a cell of a non-human organism or a part thereof, preferably a microorganism, a plant cell, a plant or a part thereof, as compared to a corresponding non-transformed wild type non-human organism or a part thereof; and
[101832] (b) growing the non-human organism or a part thereof under conditions which permit the production of hentriacontane or a composition comprising hentriacontane in said non-human organism or in the culture medium surrounding said non-human organism.

[101833] Accordingly, the present invention relates to a process for the production of hentriacontane, which comprises
[101834] (a) increasing or generating the activity of a ethanolamine utilization protein in the cytosol of a cell of a non-human organism or a part thereof, preferably a microorganism, a plant cell, a plant or a part thereof, as compared to a corresponding non-transformed wild type non-human organism or a part thereof; and
[101835] (b) growing the non-human organism or a part thereof under conditions which permit the production of hentriacontane or a composition comprising hentriacontane in said non-human organism or in the culture medium surrounding said non-human organism.

[101836] Accordingly, the present invention relates to a process for the production of hentriacontane, which comprises
[101837] (a) increasing or generating the activity of a sigma-54 dependent response regulator in the cytosol of a cell of a non-human organism or a part thereof, preferably a microorganism, a plant cell, a plant or a part thereof, as compared to a corresponding non-transformed wild type non-human organism or a part thereof; and
[101838] (b) growing the non-human organism or a part thereof under conditions which permit the production of hentriacontane or a composition comprising hentriacontane in said non-human organism or in the culture medium surrounding said non-human organism.

[101839] Accordingly, the present invention relates to a process for the production of hentriacontane, which comprises
[101840] (a) increasing or generating the activity of a b2653-protein in the cytosol of a cell of a non-human organism or a part thereof, preferably a microorganism, a plant cell, a plant or a part thereof, as compared to a corresponding non-transformed wild type non-human organism or a part thereof; and
[101841] (b) growing the non-human organism or a part thereof under conditions which permit the production of hentriacontane or a composition comprising hentriacontane in said non-human organism or in the culture medium surrounding said non-human organism.

[101842] Accordingly, the present invention relates to a process for the production of hentriacontane, which comprises
[101843] (a) increasing or generating the activity of a phosphoadenosine phosphosulfate reductase in the cytosol of a cell of a non-human organism or a part thereof, preferably a microorganism, a plant cell, a plant or a part thereof, as compared to a corresponding non-transformed wild type non-human organism or a part thereof; and
[101844] (b) growing the non-human organism or a part thereof under conditions which permit the production of hentriacontane or a composition comprising hentriacontane in said non-human organism or in the culture medium surrounding said non-human organism.

[101845] Accordingly, the present invention relates to a process for the production of hentriacontane, which comprises
[101846] (a) increasing or generating the activity of a amino-acid acetyltransferase in the cytosol of a cell of a non-human organism or a part thereof, preferably a microorganism, a plant cell, a plant or a part thereof, as compared to a corresponding non-transformed wild type non-human organism or a part thereof; and
[101847] (b) growing the non-human organism or a part thereof under conditions which permit the production of hentriacontane or a composition comprising hentriacontane in said non-human organism or in the culture medium surrounding said non-human organism.

[101848] Accordingly, the present invention relates to a process for the production of hentriacontane, which comprises
[101849] (a) increasing or generating the activity of a exonuclease subunit in the cytosol of a cell of a non-human organism or a part thereof, preferably a microorganism, a plant cell, a plant or a part thereof, as compared to a corresponding non-transformed wild type non-human organism or a part thereof; and
[101850] (b) growing the non-human organism or a part thereof under conditions which permit the production of hentriacontane or a composition comprising hentriacontane in said non-human organism or in the culture medium surrounding said non-human organism.

[101851] Accordingly, the present invention relates to a process for the production of hentriacontane, which comprises
[101852] (a) increasing or generating the activity of a L-asparaginase in the cytosol of a cell of a non-human organism or a part thereof, preferably a microorganism, a plant cell, a plant or a part thereof, as compared to a corresponding non-transformed wild type non-human organism or a part thereof; and
[101853] (b) growing the non-human organism or a part thereof under conditions which permit the production of hentriacontane or a composition comprising hentriacontane in said non-human organism or in the culture medium surrounding said non-human organism.

[101854] Accordingly, the present invention relates to a process for the production of hentriacontane, which comprises
[101855] (a) increasing or generating the activity of a ornithine decarboxylase in the cytosol of a cell of a non-human organism or a part thereof, preferably a microorganism, a plant cell, a plant or a part thereof, as compared to a corresponding non-transformed wild type non-human organism or a part thereof; and
[101856] (b) growing the non-human organism or a part thereof under conditions which permit the production of hentriacontane or a composition comprising hentriacontane in said non-human organism or in the culture medium surrounding said non-human organism.

[101857] Accordingly, the present invention relates to a process for the production of hentriacontane, which comprises
[101858] (a) increasing or generating the activity of a phosphoglycolate phosphatase in the cytosol of a cell of a non-human organism or a part thereof, preferably a microorganism, a plant cell, a plant or a part thereof, as compared to a corresponding non-transformed wild type non-human organism or a part thereof; and
[101859] (b) growing the non-human organism or a part thereof under conditions which permit the production of hentriacontane or a composition comprising hentriacontane in said non-human organism or in the culture medium surrounding said non-human organism.

[101860] Accordingly, the present invention relates to a process for the production of hentriacontane, which comprises
[101861] (a) increasing or generating the activity of a sugar transport system permease protein in the cytosol of a cell of a non-human organism or a part thereof, preferably a microorganism, a plant cell, a plant or a part thereof, as compared to a corresponding non-transformed wild type non-human organism or a part thereof; and
[101862] (b) growing the non-human organism or a part thereof under conditions which permit the production of hentriacontane or a composition comprising hentriacontane in said non-human organism or in the culture medium surrounding said non-human organism.

[101863] Accordingly, the present invention relates to a process for the production of hentriacontane, which comprises
[101864] (a) increasing or generating the activity of a cyclin D in the cytosol of a cell of a non-human organism or a part thereof, preferably a microorganism, a plant cell, a plant or a part thereof, as compared to a corresponding non-transformed wild type non-human organism or a part thereof; and
[101865] (b) growing the non-human organism or a part thereof under conditions which permit the production of hentriacontane or a composition comprising hentriacontane in said non-human organism or in the culture medium surrounding said non-human organism.

[101866] Accordingly, the present invention relates to a process for the production of hentriacontane, which comprises
[101867] (a) increasing or generating the activity of a short-chain alcohol dehydrogenase family in the cytosol of a cell of a non-human organism or a part thereof, preferably a microorganism, a plant cell, a plant or a part thereof, as compared to a corresponding non-transformed wild type non-human organism or a part thereof; and
[101868] (b) growing the non-human organism or a part thereof under conditions which permit the production of hentriacontane or a composition comprising hentriacontane in said non-human organism or in the culture medium surrounding said non-human organism.

[101869] Accordingly, the present invention relates to a process for the production of hentriacontane, which comprises
[101870] (a) increasing or generating the activity of a cell division protein in the cytosol of a cell of a non-human organism or a part thereof, preferably a microorganism, a plant cell, a plant or a part thereof, as compared to a corresponding non-transformed wild type non-human organism or a part thereof; and
[101871] (b) growing the non-human organism or a part thereof under conditions which permit the production of hentriacontane or a composition comprising hentriacontane in said non-human organism or in the culture medium surrounding said non-human organism.

[101872] Accordingly, the present invention relates to a process for the production of hentriacontane, which comprises
[101873] (a) increasing or generating the activity of a metal-dependent hydrolase in the cytosol of a cell of a non-human organism or a part thereof, preferably a microorganism, a plant cell, a plant or a part thereof, as compared to a corresponding non-transformed wild type non-human organism or a part thereof; and
[101874] (b) growing the non-human organism or a part thereof under conditions which permit the production of hentriacontane or a composition comprising hentriacontane in said non-human organism or in the culture medium surrounding said non-human organism.

[101875] Accordingly, the present invention relates to a process for the production of hentriacontane, which comprises
[101876] (a) increasing or generating the activity of a 3-deoxy-7-phosphoheptulonate synthase in the cytosol of a cell of a non-human organism or a part thereof, preferably a microorganism, a plant cell, a plant or a part thereof, as compared to a corresponding non-transformed wild type non-human organism or a part thereof; and
[101877] (b) growing the non-human organism or a part thereof under conditions which permit the production of hentriacontane or a composition comprising hentriacontane in said non-human organism or in the culture medium surrounding said non-human organism.

[101878] Accordingly, the present invention relates to a process for the production of hentriacontane, which comprises
[101879] (a) increasing or generating the activity of a YFL019C-protein in the cytosol of a cell of a non-human organism or a part thereof, preferably a microorganism, a plant cell, a plant or a part thereof, as compared to a corresponding non-transformed wild type non-human organism or a part thereof; and
[101880] (b) growing the non-human organism or a part thereof under conditions which permit the production of hentriacontane or a composition comprising hentriacontane in said non-human organism or in the culture medium surrounding said non-human organism.

[101881] Accordingly, the present invention relates to a process for the production of hentriacontane, which comprises
[101882] (a) increasing or generating the activity of a dihydroxyacetone kinase in the cytosol of a cell of a non-human organism or a part thereof, preferably a microorganism, a plant cell, a plant or a part thereof, as compared to a corresponding non-transformed wild type non-human organism or a part thereof; and
[101883] (b) growing the non-human organism or a part thereof under conditions which permit the production of hentriacontane or a composition comprising hentriacontane in said non-human organism or in the culture medium surrounding said non-human organism.

[101884] Accordingly, the present invention relates to a process for the production of hentriacontane, which comprises
[101885] (a) increasing or generating the activity of a methionine permease in the cytosol of a cell of a non-human organism or a part thereof, preferably a microorganism, a plant cell, a plant or a part thereof, as compared to a corresponding non-transformed wild type non-human organism or a part thereof; and
[101886] (b) growing the non-human organism or a part thereof under conditions which permit the production of hentriacontane or a composition comprising hentriacontane in said non-human organism or in the culture medium surrounding said non-human organism.

[101887] Accordingly, the present invention relates to a process for the production of hentriacontane, which comprises
[101888] (a) increasing or generating the activity of a YHL039W-protein in the cytosol of a cell of a non-human organism or a part thereof, preferably a microorganism, a plant cell, a plant or a part thereof, as compared to a corresponding non-transformed wild type non-human organism or a part thereof; and
[101889] (b) growing the non-human organism or a part thereof under conditions which permit the production of hentriacontane or a composition comprising hentriacontane in said non-human organism or in the culture medium surrounding said non-human organism.

[101890] Accordingly, the present invention relates to a process for the production of hentriacontane, which comprises
[101891] (a) increasing or generating the activity of a YHR022C-A-protein in the cytosol of a cell of a non-human organism or a part thereof, preferably a microorganism, a plant cell, a plant or a part thereof, as compared to a corresponding non-transformed wild type non-human organism or a part thereof; and
[101892] (b) growing the non-human organism or a part thereof under conditions which permit the production of hentriacontane or a composition comprising hentriacontane in said non-human organism or in the culture medium surrounding said non-human organism.

[101893] Accordingly, the present invention relates to a process for the production of hentriacontane, which comprises
[101894] (a) increasing or generating the activity of a YJL127W-A-protein in the cytosol of a cell of a non-human organism or a part thereof, preferably a microorganism, a plant cell, a plant or a part thereof, as compared to a corresponding non-transformed wild type non-human organism or a part thereof; and
[101895] (b) growing the non-human organism or a part thereof under conditions which permit the production of hentriacontane or a composition comprising hentriacontane in said non-human organism or in the culture medium surrounding said non-human organism.

[101896] Accordingly, the present invention relates to a process for the production of hentriacontane, which comprises
[101897] (a) increasing or generating the activity of a ylr157c-protein in the cytosol of a cell of a non-human organism or a part thereof, preferably a microorganism, a plant cell, a plant or a part thereof, as compared to a corresponding non-transformed wild type non-human organism or a part thereof; and
[101898] (b) growing the non-human organism or a part thereof under conditions which permit the production of hentriacontane or a composition comprising hentriacontane in said non-human organism or in the culture medium surrounding said non-human organism.

[101899] Accordingly, the present invention relates to a process for the production of hentriacontane, which comprises
[101900] (a) increasing or generating the activity of a ynl321w-protein in the cytosol of a cell of a non-human organism or a part thereof, preferably a microorganism, a plant cell, a plant or a part thereof, as compared to a corresponding non-transformed wild type non-human organism or a part thereof; and
[101901] (b) growing the non-human organism or a part thereof under conditions which permit the production of hentriacontane or a composition comprising hentriacontane in said non-human organism or in the culture medium surrounding said non-human organism.

[101902] Accordingly, the present invention relates to a process for the production of hentriacontane, which comprises
[101903] (a) increasing or generating the activity of a ypr170c-protein in the cytosol of a cell of a non-human organism or a part thereof, preferably a microorganism, a plant cell, a plant or a part thereof, as compared to a corresponding non-transformed wild type non-human organism or a part thereof; and
[101904] (b) growing the non-human organism or a part thereof under conditions which permit the production of hentriacontane or a composition comprising hentriacontane in said non-human organism or in the culture medium surrounding said non-human organism.

[101905] Accordingly, the present invention relates to a process for the production of hentriacontane, which comprises
[101906] (a) increasing or generating the activity of a YPR172W-protein in the cytosol of a cell of a non-human organism or a part thereof, preferably a microorganism, a plant cell, a plant or a part thereof, as compared to a corresponding non-transformed wild type non-human organism or a part thereof; and
[101907] (b) growing the non-human organism or a part thereof under conditions which permit the production of hentriacontane or a composition comprising hentriacontane in said non-human organism or in the culture medium surrounding said non-human organism.

[101908] Accordingly, the present invention relates to a process for the production of hentriacontane, which comprises
[101909] (a) increasing or generating the activity of a b3644-protein in the cytosol of a cell of a non-human organism or a part thereof, preferably a microorganism, a plant cell, a plant or a part thereof, as compared to a corresponding non-transformed wild type non-human organism or a part thereof; and
[101910] (b) growing the non-human organism or a part thereof under conditions which permit the production of hentriacontane or a composition comprising hentriacontane in said non-human organism or in the culture medium surrounding said non-human organism.

[101911] Accordingly, the present invention relates to a process for the production of hentriacontane, which comprises
[101912] (a) increasing or generating the activity of a polypeptide comprising a polypeptide as depicted in the respective line in column 5 or 8 of Table II, application no. 20, or a homolog or a fragment thereof, a consensus sequence or at least one polypeptide motif as depicted in the respective line in column 8 of Table IV, application no. 20, whereby the respective line disclose in column 7 the fine chemical hentriacontane; or
[101913] increasing or generating the activity of an expression product of one or more nucleic acid molecule(s) comprising a polynucleotide as depicted in the respective line in column 5 or 8 of Table I, application no. 20, preferably the coding region thereof, or a homolog or a fragment thereof, whereby the respective line disclose in column 7 the fine chemical hentriacontane;
[101914] in the cytosol of a cell of a non-human organism or a part thereof; preferably a microorganism, a plant cell, a plant or a part thereof, as compared to a corresponding non-transformed wild type non-human organism or a part thereof; and
[101915] (b) growing the non-human organism under conditions which permit the production of hentriacontane, or a composition comprising hentriacontane in said non-human organism or in the culture medium surrounding said non-human organism.

[101916] In a further embodiment the activity of the polypeptide comprising a polypeptide as depicted in the respective line in column 5 or 8 of Table II, application no. 20, or a homolog or a fragment thereof, a consensus sequence or at least one polypeptide motif as depicted in the respective line in column 8 of Table IV, application no. 20, is increased or generated non-targeted in the above-mentioned process in a microorganism or plant or a part thereof.

[101917] In a further embodiment said polypeptide has the activity of the respective polypeptide represented by a protein comprising a polypeptide as depicted in the respective line in column 5 of Table II, application no. 20.

[101918] In a further embodiment the activity of the expression product of one or more nucleic acid molecule(s) comprising a polynucleotide as depicted in the respective line in column 5 or 8 of Table I, application no. 20, preferably the coding region thereof, or a homolog or a fragment thereof, is increased or generated non-targeted in the above-mentioned process in a microorganism or plant or a part thereof.

[101919] In a further embodiment the activity of the polypeptide comprising a polypeptide as depicted in the respective line in column 5 or 8 of Table II, application no. 20, or a homolog or a fragment thereof, a consensus sequence or at least one polypeptide motif as depicted in the respective line in column 8 of Table IV, application no. 20, is increased or generated in the above-mentioned process in an organelle preferably in plastids or mitochondria, especially in plastids, of a microorganism or plant.

[101920] In a further embodiment said polypeptide has the activity of the respective polypeptide represented by a protein comprising a polypeptide as depicted in the respective line in column 5 of Table II, application no. 20.

[101921] In a further embodiment the activity of the expression product of one or more nucleic acid molecule(s) comprising a polynucleotide as depicted in the respective line in column 5 or 8 of Table I, application no. 20, preferably the coding region thereof, or a homolog or a fragment thereof, is increased or generated in the above-mentioned process in an organelle, preferably in plasids or mitochondria, especially in plastids, of a microorganism or plant.

[101922] In a further embodiment the activity of the polypeptide comprising a polypeptide as depicted in the respective line in column 5 or 8 of Table II, application no. 20, or a homolog or a fragment thereof, a consensus sequence or at least one polypeptide motif as depicted in the respective line in column 8 of Table IV, application no. 20, is increased or generated in the above-mentioned process in the cyctosol of a cell, of a microorganism or plant.

[101923] In a further embodiment said polypeptide has the activity of the respective polypeptide represented by a protein comprising a polypeptide as depicted in the respective line in column 5 of Table II, application no. 20.

[101924] In a further embodiment the activity of the expression product of one or more nucleic acid molecule(s) comprising a polynucleotide as depicted in the respective line in column 5 or 8 of Table I, application no. 20, preferably the coding region thereof, or a homolog or a fragment thereof, is increased or generated in the above-mentioned process in the cytosol of a cell, of a microorganism or plant.

[101925] [0039.1.7.20] to [0066.1.7.20] for the disclosure of these paragraphs see [0039.1.7.7] to [0066.1.7.7] above

[101926] As mentioned above the nucleic acid sequences coding for the proteins as shown in Table II, application no. 20, column 5 or 8, or homologs or fragments thereof, are joined to a nucleic acid sequence encoding a plastidic transit peptide, e.g, if for the nucleic acid molecule in Table I, column 6 the term “plastidic” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide coding sequence is fused in frame to the nucleic acid sequence coding for proteins as shown in Table II, application no. 20, column 5 or 8, or homologs or fragments thereof, e.g., if for the nucleic acid molecule in Table I, column 6 the term “plastidic” is indicated.

[101927] [0068.1.7.20] to [0072.1.7.20] for the disclosure of these paragraphs see [0068.1.7.7] to [0072.1.7.7] above

[101928] As mentioned above the nucleic acid sequences coding for the proteins as shown in the respective line in Table II, application no. 20, column 5 or 8, or homologs or fragments thereof, are joined to a nucleic acid sequence encoding a mitochondric transit peptide, e.g., if for the respective nucleic acid molecule in Table I, column 6 the term “mitochondric” is indicated. This nucleic acid sequence encoding a transit peptide ensures transport of the protein to the organelle. The nucleic acid sequence of the gene to be expressed and the nucleic acid sequence encoding the transit peptide are operably linked. Therefore the transit peptide encoding sequence is fused in frame to the nucleic acid sequence coding for proteins as shown in the respective line in Table II, application no. 20, column 5 or 8, or homologs or fragments thereof, e.g., if for the nucleic acid molecule in the respective line in Table I, column 6 the term “mitochondric” is indicated.

[101929] for the disclosure of this paragraph see [0074.1.7.7] above

[101930] for the disclosure of this paragraph see [0075.1.7.7] above

[101931] Nucleic acid sequences coding for the transit peptides may be chemically synthesized either in part or wholly according to structure of transit peptide sequences disclosed in the prior art. Said natural or chemically synthesized sequences can be directly linked to the sequences encoding the mature protein or via a linker nucleic acid sequence, which may be typically less than 500 base pairs, preferably less than 450, 400, 350, 300, 250 or 200 base pairs, more preferably less than 150, 100, 90, 80, 70, 60, 50, 40 or 30 base pairs and most preferably less than 25, 20, 15, 12, 9, 6 or 3 base pairs in length and are in frame to the coding sequence.

[101932] Furthermore favorable nucleic acid sequences encoding transit peptides may comprise sequences derived from more than one biological and/or chemical source and may include a nucleic acid sequence derived from the amino-terminal region of the mature protein, which in its native state is linked to the transit peptide. In a preferred embodiment of the invention said amino-terminal region of the mature protein is typically less than 150 amino acids, preferably less than 140, 130, 120, 110, 100 or 90 amino acids, more preferably less than 80, 70, 60, 50, 40, 35, 30, 25 or 20 amino acids and most preferably less than 19, 18, 17, 16, 15, 14, 13, 12, 11 or 10 amino acids in length. But even shorter or longer stretches are also possible. In addition target sequences, which facilitate the transport of proteins to other cell compartments such as the vacuole, endoplasmic reticulum, golgi complex, glyoxysomes, peroxisomes, mitochondria (in case of a plastidal transit peptide) or plastids, especially chloroplasts (in case of a mitochondric transit peptide) may be also part of the inventive nucleic acid sequence. The proteins translated from said inventive nucleic acid sequences are a kind of fusion proteins that means the nucleic acid sequences encoding the plastidal transit peptide for example the ones shown in Table a, preferably the last one of the Table a are joint to the respective nucleic acid sequences shown in Table I, application no. 20, columns 5 or 8, especially the coding region thereof, or homologs or fragments thereof, in case of nucleic acid sequences being expressed in plastids, and that means the nucleic acid sequences encoding the mitochondric transit peptide for example the ones shown in Table b, preferably the last one of the Table b are joint to the respective nucleic acid sequences shown in Table I, application no. 20, columns 5 or 8, especially the coding region thereof, or homologs or fragments thereof, in case of nucleic acid sequences being expressed in mitochondria. The person skilled in the art is able to join said sequences in a functional manner. Advantageously the transit peptide part is cleaved off from the protein part shown in Table II, application no. 20, columns 5 or 8, or a homolog or fragment thereof, during or after the transport into the organelles, preferably into the plastids or mitochondria, respectively.

[101933] All products of the cleavage of the preferred transit peptide shown in the last line of Table a have preferably the N-terminal amino acid sequences QIA CSS or QIA EFQLTT in front of the start methionine of the respective protein mentioned in Table II, application no. 20, column 5 or 8. or homologs or fragments thereof. Other short amino acid sequences of an range of 1 to 20 amino acids preferable 2 to 15 amino acids, more preferable 3 to 10 amino acids most preferably 4 to 8 amino acids are also possible in front of the start methionine of the protein mentioned in Table II, application no. 20, columns 5 or 8, or homologs or fragments thereof. In case of the amino acid sequence QIA CSS the three amino acids in front of the start methionine are stemming from the LIC (=ligatation independent cloning) cassette. Said short amino acid sequence is preferred in the case of the expression of E. coli genes. In case of the amino acid sequence QIA EFQLTT the six amino acids in front of the start methionine are stemming from the LIC cassette. Said short amino acid sequence is preferred in the case of the expression of S. cerevisiae genes. The skilled worker knowns that other short sequences are also useful in the expression of the respective nucleic acid molecule comprising a polynucleotide as depicted in Table I, application no. 20, column 5 or 8, preferably the coding region thereof, or a homolog or a fragment thereof. Furthermore the skilled worker is aware of the fact that there is not a need for such short sequences in the expression of the genes.

[101934] for the disclosure of this paragraph see [0077.1.7.7] above

[101935] for the disclosure of this paragraph see [0078.1.7.7] above

[101936] Alternatively to the targeting of the respective sequences shown in Table II, application no. 20, columns 5 or 8, or homologs or fragments thereof, preferably of sequences in general encoded in the nucleus with the aid of the targeting sequences mentioned for example in Table a alone, Table b alone, or in combination with other targeting sequences preferably into plastids or mitochondria, respectively, the nucleic acids of the invention can directly be introduced into the plastidal or mitochondrial genome. Therefore in a preferred embodiment the respective nucleic acid sequences shown in the respective line in Table I, application no. 20, columns 5 or 8, preferably the coding region thereof, or homologs or fragments thereof, are directly introduced and expressed in plastids e.g., if for the nucleic acid molecule in the respective line in Table I, column 6, the term “plastidic” is indicated, or are directly introduced and expressed in mitochondria e.g., if for the nucleic acid molecule in the respective line in Table I, column 6 the term “mitochondric” is indicated.

[101937] The term “introduced” in the context of this specification shall mean the insertion of a nucleic acid sequence into the organism by means of a “transfection”, “transduction” or preferably by “transformation”.

[101938] A plastid, such as a chloroplast, or a mitochondrion, has been “transformed” by an exogenous (preferably foreign) nucleic acid sequence if a nucleic acid sequence has been introduced into the plastid or mitochondrion that means that this sequence has crossed the membrane or the membranes of the plastid or of the mitochondrion. The exogenous, preferably foreign, DNA may be integrated (covalently linked) into plastid or mitochondrial DNA making up the genome of the plastid or mitochondrion, or it may remain unintegrated (e.g., by including a chloroplast origin of replication). “Stably” integrated DNA sequences are those, which are inherited through plastid or mitochondrion replication, thereby transferring new plastids or mitochondria, with the features of the integrated DNA sequence to the progeny.

[101939] [0080.1.7.20] to [0083.1.7.20] for the disclosure of these paragraphs see [0080.1.7.7] to [0083.1.7.7] above

[101940] For the process of the present invention it may be of great advantage that by transforming the plastids the intraspecies specific transgene flow is blocked, because a lot of species such as corn, cotton and rice have a strict maternal inheritance of plastids. By placing the respective genes specified in the respective line in Table I, application no. 20, columns 5 or 8, preferably the coding region, or homologs or fragments thereof, e.g. if for the nucleic acid molecule in the respective line in column 6 of Table I the term “plastidic” is indicated, in the plastids of plants, these genes will not be present in the pollen of said plants.

[101941] A further preferred embodiment of the invention relates to the use of so called “chloroplast localization sequences”, in which a first RNA sequence or molecule is capable of transporting or “chaperoning” a second RNA sequence, such as a RNA sequence transcribed from the sequences depicted in the respective line in Table I, application no. 20, columns 5 or 8, preferably the coding region thereof, or a homolog or a fragment thereof, or a sequence encoding a protein, as depicted in the respective line in Table II, application no. 20, columns 5 and 8, or a homolog or a fragment thereof, from an external environment inside a cell or outside a plastid into a chloroplast. In one embodiment the chloroplast localization signal is substantially similar or complementary to a complete or intact viroid sequence. The chloroplast localization signal may be encoded by a DNA sequence, which is transcribed into the chloroplast localization RNA.

[101942] The term “viroid” refers to a naturally occurring single stranded RNA molecule (Flores, C. R. Acad. Sci. III. 324 (10), 943 (2001)). Viroids usually contain about 200-500 nucleotides and generally exist as circular molecules. Examples of viroids that contain chloroplast localization signals include but are not limited to ASBVd, PLMVd, CChMVd and ELVd. The viroid sequence or a functional part of it can be fused to the sequences depicted in the respective line in Table I, application no. 20, columns 5 or 8, preferably the coding region thereof, or a homolog or a fragment thereof, or a sequence encoding a protein, as depicted in the respective line in Table II, application no. 20, columns 5 or 8, or a homolog or a fragment thereof, in such a manner that the viroid sequence transports a sequence transcribed from a sequence as depicted in the respective line in Table I, application no. 20 columns 5 or 8, preferably the coding region thereof, or a homolog or a fragment thereof, or a sequence encoding a protein as depicted in the respective line in Table II, application no. 20 columns 5 or 8, or a homolog or a fragment thereof, into the chloroplasts, e.g. if for the nucleic acid molecule in column 6 of Table I the term “plastidic” is indicated. A preferred embodiment uses a modified ASBVd (Navarro et al., Virology. 268 (1), 218 (2000)).

[101943] In a further specific embodiment the protein to be expressed in the plastids such as the proteins depicted in the respective line in Table II, application no. 20, columns 5 or 8, or a homolog or a fragment thereof, e.g. if for the nucleic acid molecule in the respective line in column 6 of Table I the term “plastidic” is indicated, are encoded by different nucleic acids. Such a method is disclosed in WO 2004/040973, which shall be incorporated by reference. WO 2004/040973 teaches a method, which relates to the translocation of an RNA corresponding to a gene or gene fragment into the chloroplast by means of a chloroplast localization sequence.

[101944] The genes, which should be expressed in the plant or plant cells, are split into nucleic acid fragments, which are introduced into different compartments in the plant e.g. the nucleus, the plastids and/or mitochondria. Additionally plant cells are described in which the chloroplast contains a ribozyme fused at one end to an RNA encoding a fragment of a protein used in the inventive process such that the ribozyme can trans-splice the translocated fusion RNA to the RNA encoding the gene fragment to form and as the case may be reunite the nucleic acid fragments to an intact mRNA encoding a functional protein for example as disclosed in the respective line in Table II, application no. 20, columns 5 or 8, or a homolog or a fragment thereof.

[101945] In a preferred embodiment of the invention the nucleic acid sequences as shown in the respective line in Table I, application no. 20, columns 5 or 8, preferably the coding region thereof, or homologs or fragments thereof, e.g. if for the nucleic acid molecule in the respective line in column 6 of Table I the term “plastidic” is indicated, used in the inventive process are transformed into plastids, which are metabolically active. Those plastids should preferably maintain at a high copy number in the plant or plant tissue of interest, most preferably the chloroplasts found in green plant tissues, such as leaves or cotyledons or in seeds.

[101946] For a good expression in the plastids the nucleic acid sequences as shown in the respective line in Table I, application no. 20, columns 5 or 8, preferably the coding region thereof, or homologs or fragments thereof, e.g. if for the nucleic acid molecule in the respective line in column 6 of Table I the term “plastidic” is indicated, are introduced into an expression cassette using preferably a promoter and terminater, which are active in plastids preferably a chloroplast promoter. Examples of such promoters include the psbA promoter from the gene from spinach or pea, the rbcL promoter, and the atpB promoter from corn.

[101947] [0089.1.7.20] to [0092.1.7.20] for the disclosure of these paragraphs see [0089.1.7.7] to [0092.1.7.7] above

[101948] Advantageously the process for the production of the fine chemical hentriacontane leads to an enhanced production of the respective fine chemical. The terms “enhanced” or “increase” mean at least a 10%, 20%, 30%, 40% or 50%, preferably at least 60%, 70%, 80%, 90% or 100%, more preferably 150%, 200%, 300%, 400% or 500% higher production of the respective fine chemical hentriacontane in comparison to the wild-type as defined above, e.g. that means in comparison to a non-human organism without the aforementioned modification of the activity of a protein as shown in the respective line in Table II, application no. 20, column 5 or 8, or a fragment or a homolog thereof. The modification of the activity of a protein as shown in the respective line in Table II, application no. 20, column 5 or 8, or a homolog or a fragment thereof, or their combination can be achieved by joining the protein to a respective transit peptide, e.g. if for the respective encoding nucleic acid molecule in column 6 of Table I the term “plastidic” or “mitochondric” is indicated.

[101949] Surprisingly it was found, that the transgenic expression of a protein as shown in the respective line in Table II, application no. 20, column 5, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, conferred a production of or an increase in hentriacontane,respectively, to the transgenic non-human organism as compared to a corresponding non-transformed wild type.

[101950] In one embodiment the transgenic expression of a protein as shown in the respective line in Table II, application no. 20, column 5 or 8, or a homolog or a fragment thereof, especially from the Saccaromyces cerevisiae, E. coli, Arabidopsis thaliana, Azotobacter vinelandii, Brassica napus, Glycine max, Oryza sativa, Physcomitrella patens, Synechocystis sp., Thermus thermophilus, Zea mays in a non-human organism, like a plant or a part thereof, such as Arabidopsis thaliana for example, or a microorganism, confers a production of or an increase in hentriacontane,respectively, to the transgenic non-human organism as compared to a corresponding non-transformed wild type.

[101951] Accordingly, in one embodiment, an increase of hentriacontane in a non-human organism, as compared to a corresponding non-transformed wild type non-human organism, is conferred in the process of the invention, if the activity of a polypeptide showing the activity of a pyruvate kinase, or if the activity of the polypeptide At2g36580, preferably represented by SEQ ID NO. 113715, or a homolog or fragment thereof, or if the activity of a polypeptide encoded by a nucleic acid molecule comprising the nucleic acid SEQ ID NO. 113714, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 113714 or polypeptide SEQ ID NO. 113715, respectively, or a homolog or a fragment thereof, is increased or generated, or if the activity pyruvate kinase is increased or generated in a non-human organism, like a microorganism or a plant cell, plant or part thereof, especially with plastidic localization, whereby the respective line disclose in column 7 the fine chemical hentriacontane. For example, an increase of the hentriacontane of at least 1 percent, particularly in a range of 28 to 48-percent is conferred as compared to a corresponding non-transformed wild type non-human organism.

[101952] Accordingly, in one embodiment, an increase of hentriacontane in a non-human organism, as compared to a corresponding non-transformed wild type non-human organism, is conferred in the process of the invention, if the activity of a polypeptide showing the activity of a MADS box protein transcription factor, or if the activity of the polypeptide At2g42830, preferably represented by SEQ ID NO. 108728, or a homolog or fragment thereof, or if the activity of a polypeptide encoded by a nucleic acid molecule comprising the nucleic acid SEQ ID NO. 108727, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 108727 or polypeptide SEQ ID NO. 108728, respectively, or a homolog or a fragment thereof, is increased or generated, or if the activity MADS box protein transcription factor is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 49 to 90-percent is conferred as compared to a corresponding non-transformed wild type non-human organism.

[101953] Accordingly, in one embodiment, an increase of hentriacontane in a non-human organism, as compared to a corresponding non-transformed wild type non-human organism, is conferred in the process of the invention, if the activity of a polypeptide showing the activity of a At2g45420-protein, or if the activity of the polypeptide At2g45420, preferably represented by SEQ ID NO. 1624, or a homolog or fragment thereof, or if the activity of a polypeptide encoded by a nucleic acid molecule comprising the nucleic acid SEQ ID NO. 1623, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 1623 or polypeptide SEQ ID NO. 1624, respectively, or a homolog or a fragment thereof, is increased or generated, or if the activity At2g45420-protein is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 34 to 100-percent is conferred as compared to a corresponding non-transformed wild type non-human organism.

[101954] Accordingly, in one embodiment, an increase of hentriacontane in a non-human organism, as compared to a corresponding non-transformed wild type non-human organism, is conferred in the process of the invention, if the activity of a polypeptide showing the activity of a transcription factor, or if the activity of the polypeptide At4g09960, preferably represented by SEQ ID NO. 109718, or a homolog or fragment thereof, or if the activity of a polypeptide encoded by a nucleic acid molecule comprising the nucleic acid SEQ ID NO. 109717, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 109717 or polypeptide SEQ ID NO. 109718, respectively, or a homolog or a fragment thereof, is increased or generated, or if the activity transcription factor is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 27 to 231-percent is conferred as compared to a corresponding non-transformed wild type non-human organism.

[101955] Accordingly, in one embodiment, an increase of hentriacontane in a non-human organism, as compared to a corresponding non-transformed wild type non-human organism, is conferred in the process of the invention, if the activity of a polypeptide showing the activity of a At5g43630-protein, or if the activity of the polypeptide At5g43630, preferably represented by SEQ ID NO. 142930, or a homolog or fragment thereof, or if the activity of a polypeptide encoded by a nucleic acid molecule comprising the nucleic acid SEQ ID NO. 142929, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 142929 or polypeptide SEQ ID NO. 142930, respectively, or a homolog or a fragment thereof, is increased or generated, or if the activity At5g43630-protein is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 26 to 37-percent is conferred as compared to a corresponding non-transformed wild type non-human organism.

[101956] Accordingly, in one embodiment, an increase of hentriacontane in a non-human organism, as compared to a corresponding non-transformed wild type non-human organism, is conferred in the process of the invention, if the activity of a polypeptide showing the activity of a RNA-binding protein, or if the activity of the polypeptide At5g60110, preferably represented by SEQ ID NO. 70007, or a homolog or fragment thereof, or if the activity of a polypeptide encoded by a nucleic acid molecule comprising the nucleic acid SEQ ID NO. 70006, preferably the coding region thereof, or a homolog or fragment thereof, e.g. derived from Arabidopsis thaliana, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 70006 or polypeptide SEQ ID NO. 70007, respectively, or a homolog or a fragment thereof, is increased or generated, or if the activity RNA-binding protein is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 28 to 97-percent is conferred as compared to a corresponding non-transformed wild type non-human organism.

[101957] Accordingly, in one embodiment, an increase of hentriacontane in a non-human organism, as compared to a corresponding non-transformed wild type non-human organism, is conferred in the process of the invention, if the activity of a polypeptide showing the activity of a transcriptional regulator, or if the activity of the polypeptide AvinDRAFT_{—}3209, preferably represented by SEQ ID NO. 29287, or a homolog or fragment thereof, or if the activity of a polypeptide encoded by a nucleic acid molecule comprising the nucleic acid SEQ ID NO. 29286, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 29286 or polypeptide SEQ ID NO. 29287, respectively, or a homolog or a fragment thereof, is increased or generated, or if the activity transcriptional regulator is increased or generated in a non-human organism, like a microorganism or a plant cell, plant or part thereof, especially with non-targeted localization, whereby the respective line disclose in column 7 the fine chemical hentriacontane.

[101958] For example, an increase of the hentriacontane of at least 1 percent, particularly in a range of 55 to 116-percent is conferred as compared to a corresponding non-transformed wild type non-human organism.

[101959] Accordingly, in one embodiment, an increase of hentriacontane in a non-human organism, as compared to a corresponding non-transformed wild type non-human organism, is conferred in the process of the invention, if the activity of a polypeptide showing the activity of a fumarylacetoacetate hydrolase, or if the activity of the polypeptide AvinDRAFT_{—}5292, preferably represented by SEQ ID NO. 32309, or a homolog or fragment thereof, or if the activity of a polypeptide encoded by a nucleic acid molecule comprising the nucleic acid SEQ ID NO. 32308, preferably the coding region thereof, or a homolog or fragment thereof, e.g. derived from Azotobacter vinelandii, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 32308 or polypeptide SEQ ID NO. 32309, respectively, or a homolog or a fragment thereof, is increased or generated, or if the activity fumarylacetoacetate hydrolase is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, partitularly in a range of 26 to 53-percent is conferred as compared to a corresponding non-transformed wild type non-human organism.

[101960] Accordingly, in one embodiment, an increase of hentriacontane in a non-human organism, as compared to a corresponding non-transformed wild type non-human organism, is conferred in the process of the invention, if the activity of a polypeptide showing the activity of a spermidine synthase, or if the activity of the polypeptide B0121, preferably represented by SEQ ID NO. 154510, or a homolog or fragment thereof, or if the activity of a polypeptide encoded by a nucleic acid molecule comprising the nucleic acid SEQ ID NO. 154509, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 154509 or polypeptide SEQ ID NO. 154510, respectively, or a homolog or a fragment thereof, is increased or generated, or if the activity spermidine synthase is increased or generated in a non-human organism, like a microorganism or a plant cell, plant or part thereof, especially with plastidic localization, whereby the respective line disclose in column 7 the fine chemical hentriacontane. For example, an increase of the hentriacontane of at least 1 percent, particularly in a range of 25 to 50-percent is conferred as compared to a corresponding non-transformed wild type non-human organism.

[101961] Accordingly, in one embodiment, an increase of hentriacontane in a non-human organism, as compared to a corresponding non-transformed wild type non-human organism, is conferred in the process of the invention, if the activity of a polypeptide showing the activity of a serine protease, or if the activity of the polypeptide B0161, preferably represented by SEQ ID NO. 7082, or a homolog or fragment thereof, or if the activity of a polypeptide encoded by a nucleic acid molecule comprising the nucleic acid SEQ ID NO. 7081, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 7081 or polypeptide SEQ ID NO. 7082, respectively, or a homolog or a fragment thereof, is increased or generated, or if the activity serine protease is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 26 to 69-percent is conferred as compared to a corresponding non-transformed wild type non-human organism.

[101962] Accordingly, in one embodiment, an increase of hentriacontane in a non-human organism, as compared to a corresponding non-transformed wild type non-human organism, is conferred in the process of the invention, if the activity of a polypeptide showing the activity of a peptide chain release factor, or if the activity of the polypeptide B0236, preferably represented by SEQ ID NO. 155231, or a homolog or fragment thereof, or if the activity of a polypeptide encoded by a nucleic acid molecule comprising the nucleic acid SEQ ID NO. 155230, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 155230 or polypeptide SEQ ID NO. 155231, respectively, or a homolog or a fragment thereof, is increased or generated, or if the activity peptide chain release factor is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 28 to 59-percent is conferred as compared to a corresponding non-transformed wild type non-human organism.

[101963] Accordingly, in one embodiment, an increase of hentriacontane in a non-human organism, as compared to a corresponding non-transformed wild type non-human organism, is conferred in the process of the invention, if the activity of a polypeptide showing the activity of a mechanosensitive ion channel, or if the activity of the polypeptide B0577, preferably represented by SEQ ID NO. 155371, or a homolog or fragment thereof, or if the activity of a polypeptide encoded by a nucleic acid molecule comprising the nucleic acid SEQ ID NO. 155370, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 155370 or polypeptide SEQ ID NO. 155371, respectively, or a homolog or a fragment thereof, is increased or generated, or if the activity mechanosensitive ion channel is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 26 to 65-percent is conferred as compared to a corresponding non-transformed wild type non-human organism.

[101964] Accordingly, in one embodiment, an increase of hentriacontane in a non-human organism, as compared to a corresponding non-transformed wild type non-human organism, is conferred in the process of the invention, if the activity of a polypeptide showing the activity of a pyrimidinespecific ribonucleoside hydrolase, or if the activity of the polypeptide B0651, preferably represented by SEQ ID NO. 143955, or a homolog or fragment thereof, or if the activity of a polypeptide encoded by a nucleic acid molecule comprising the nucleic acid SEQ ID NO. 143954, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 143954 or polypeptide SEQ ID NO. 143955, respectively, or a homolog or a fragment thereof, is increased or generated, or if the activity pyrimidine-specific ribonucleoside hydrolase is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 26 to 49-percent is conferred as compared to a corresponding non-transformed wild type non-human organism.

[101965] Accordingly, in one embodiment, an increase of hentriacontane in a non-human organism, as compared to a corresponding non-transformed wild type non-human organism, is conferred in the process of the invention, if the activity of a polypeptide showing the activity of a inner membrane protein, or if the activity of the polypeptide B0787, preferably represented by SEQ ID NO. 155614, or a homolog or fragment thereof, or if the activity of a polypeptide encoded by a nucleic acid molecule comprising the nucleic acid SEQ ID NO. 155613, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 155613 or polypeptide SEQ ID NO. 155614, respectively, or a homolog or a fragment thereof, is increased or generated, or if the activity inner membrane protein is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 24 to 107-percent is conferred as compared to a corresponding non-transformed wild type non-human organism.

[101966] Accordingly, in one embodiment, an increase of hentriacontane in a non-human organism, as compared to a corresponding non-transformed wild type non-human organism, is conferred in the process of the invention, if the activity of a polypeptide showing the activity of a b0833-protein, or if the activity of the polypeptide B0833, preferably represented by SEQ ID NO. 155658, or a homolog or fragment thereof, or if the activity of a polypeptide encoded by a nucleic acid molecule comprising the nucleic acid SEQ ID NO. 155657, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 155657 or polypeptide SEQ ID NO. 155658, respectively, or a homolog or a fragment thereof, is increased or generated, or if the activity b0833-protein is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 24 to 48-percent is conferred as compared to a corresponding non-transformed wild type non-human organism.

[101967] Accordingly, in one embodiment, an increase of hentriacontane in a non-human organism, as compared to a corresponding non-transformed wild type non-human organism, is conferred in the process of the invention, if the activity of a polypeptide showing the activity of a S-adenosyl-L-methionine-dependent methyltransferase, or if the activity of the polypeptide B0921, preferably represented by SEQ ID NO. 155666, or a homolog or fragment thereof, or if the activity of a polypeptide encoded by a nucleic acid molecule comprising the nucleic acid SEQ ID NO. 155665, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 155665 or polypeptide SEQ ID NO. 155666, respectively, or a homolog or a fragment thereof, is increased or generated, or if the activity S-adenosyl-L-methionine-dependent methyltransferase is increased or generated in a non-human organism, like a microorganism or a plant cell, plant or part thereof, especially with plastidic localization, whereby the respective line disclose in column 7 the fine chemical hentriacontane. For example, an increase of the hentriacontane of at least 1 percent, particularly in a range of 27 to 30-percent is conferred as compared to a corresponding non-transformed wild type non-human organism.

[101968] Accordingly, in one embodiment, an increase of hentriacontane in a non-human organism, as compared to a corresponding non-transformed wild type non-human organism, is conferred in the process of the invention, if the activity of a polypeptide showing the activity of a b1259-protein, or if the activity of the polypeptide B1259, preferably represented by SEQ ID NO. 37504, or a homolog or fragment thereof, or if the activity of a polypeptide encoded by a nucleic acid molecule comprising the nucleic acid SEQ ID NO. 37503, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 37503 or polypeptide SEQ ID NO. 37504, respectively, or a homolog or a fragment thereof, is increased or generated, or if the activity b1259-protein is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 27 to 72-percent is conferred as compared to a corresponding non-transformed wild type non-human organism.

[101969] Accordingly, in one embodiment, an increase of hentriacontane in a non-human organism, as compared to a corresponding non-transformed wild type non-human organism, is conferred in the process of the invention, if the activity of a polypeptide showing the activity of a orotidine-5′-phosphate decarboxylase, or if the activity of the polypeptide B1281, preferably represented by SEQ ID NO. 155891, or a homolog or fragment thereof, or if the activity of a polypeptide encoded by a nucleic acid molecule comprising the nucleic acid SEQ ID NO. 155890, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 155890 or polypeptide SEQ ID NO. 155891, respectively, or a homolog or a fragment thereof, is increased or generated, or if the activity orotidine-5′-phosphate decarboxylase is increased or generated in a non-human organism, like a microorganism or a plant cell, plant or part thereof, especially with plastidic localization, whereby the respective line disclose in column 7 the fine chemical hentriacontane. For example, an increase of the hentriacontane of at least 1 percent, particularly in a range of 41 to 48-percent is conferred as compared to a corresponding non-transformed wild type non-human organism.

[101970] Accordingly, in one embodiment, an increase of hentriacontane in a non-human organism, as compared to a corresponding non-transformed wild type non-human organism, is conferred in the process of the invention, if the activity of a polypeptide showing the activity of a ATP-dependent RNA helicase, or if the activity of the polypeptide B1343, preferably represented by SEQ ID NO. 76891, or a homolog or fragment thereof, or if the activity of a polypeptide encoded by a nucleic acid molecule comprising the nucleic acid SEQ ID NO. 76890, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 76890 or polypeptide SEQ ID NO. 76891, respectively, or a homolog or a fragment thereof, is increased or generated, or if the activity ATP-dependent RNA helicase is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 24 to 52-percent is conferred as compared to a corresponding non-transformed wild type non-human organism.

[101971] Accordingly, in one embodiment, an increase of hentriacontane in a non-human organism, as compared to a corresponding non-transformed wild type non-human organism, is conferred in the process of the invention, if the activity of a polypeptide showing the activity of a b1445-protein, or if the activity of the polypeptide B1445, preferably represented by SEQ ID NO. 38290, or a homolog or fragment thereof, or if the activity of a polypeptide encoded by a nucleic acid molecule comprising the nucleic acid SEQ ID NO. 38289, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 38289 or polypeptide SEQ ID NO. 38290, respectively, or a homolog or a fragment thereof, is increased or generated, or if the activity b1445-protein is increased or generated in a non-human organism, like a microorganism or a plant cell, plant or part thereof, especially with plastidic localization, whereby the respective line disclose in column 7 the fine chemical hentriacontane. For example, an increase of the hentriacontane of at least 1 percent, particularly in a range of 27 to 47-percent is conferred as compared to a corresponding non-transformed wild type non-human organism.

[101972] Accordingly, in one embodiment, an increase of hentriacontane in a non-human organism, as compared to a corresponding non-transformed wild type non-human organism, is conferred in the process of the invention, if the activity of a polypeptide showing the activity of a acid shock protein, or if the activity of the polypeptide B1597, preferably represented by SEQ ID NO. 38301, or a homolog or fragment thereof, or if the activity of a polypeptide encoded by a nucleic acid molecule comprising the nucleic acid SEQ ID NO. 38300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 38300 or polypeptide SEQ ID NO. 38301, respectively, or a homolog or a fragment thereof, is increased or generated, or if the activity acid shock protein is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 38 to 96-percent is conferred as compared to a corresponding non-transformed wild type non-human organism.

[101973] Accordingly, in one embodiment, an increase of hentriacontane in a non-human organism, as compared to a corresponding non-transformed wild type non-human organism, is conferred in the process of the invention, if the activity of a polypeptide showing the activity of a electron transport complex protein, or if the activity of the polypeptide B1627, preferably represented by SEQ ID NO. 38346, or a homolog or fragment thereof, or if the activity of a polypeptide encoded by a nucleic acid molecule comprising the nucleic acid SEQ ID NO. 38345, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 38345 or polypeptide SEQ ID NO. 38346, respectively, or a homolog or a fragment thereof, is increased or generated, or if the activity electron transport complex protein is increased or generated in a non-human organism, like a microorganism or a plant cell, plant or part thereof, especially with plastidic localization, whereby the respective line disclose in column 7 the fine chemical hentriacontane. For example, an increase of the hentriacontane of at least 1 percent, particularly in a range of 29 to 40-percent is conferred as compared to a corresponding non-transformed wild type non-human organism.

[101974] Accordingly, in one embodiment, an increase of hentriacontane in a non-human organism, as compared to a corresponding non-transformed wild type non-human organism, is conferred in the process of the invention, if the activity of a polypeptide showing the activity of a transcriptional regulator, or if the activity of the polypeptide B1649, preferably represented by SEQ ID NO. 156393, or a homolog or fragment thereof, or if the activity of a polypeptide encoded by a nucleic acid molecule comprising the nucleic acid SEQ ID NO. 156392, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 156392 or polypeptide SEQ ID NO. 156393, respectively, or a homolog or a fragment thereof, is increased or generated, or if the activity transcriptional regulator is increased or generated in a non-human organism, like a microorganism or a plant cell, plant or part thereof, especially with plastidic localization, whereby the respective line disclose in column 7 the fine chemical hentriacontane. For example, an increase of the hentriacontane of at least 1 percent, particularly in a range of 28 to 48-percent is conferred as compared to a corresponding non-transformed wild type non-human organism.

[101975] Accordingly, in one embodiment, an increase of hentriacontane in a non-human organism, as compared to a corresponding non-transformed wild type non-human organism, is conferred in the process of the invention, if the activity of a polypeptide showing the activity of a b1670-protein, or if the activity of the polypeptide B1670, preferably represented by SEQ ID NO. 78754, or a homolog or fragment thereof, or if the activity of a polypeptide encoded by a nucleic acid molecule comprising the nucleic acid SEQ ID NO. 78753, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 78753 or polypeptide SEQ ID NO. 78754, respectively, or a homolog or a fragment thereof, is increased or generated, or if the activity b1670-protein is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 30 to 48-percent is conferred as compared to a corresponding non-transformed wild type non-human organism.

[101976] Accordingly, in one embodiment, an increase of hentriacontane in a non-human organism, as compared to a corresponding non-transformed wild type non-human organism, is conferred in the process of the invention, if the activity of a polypeptide showing the activity of a phospho-2-dehydro-3-deoxyheptonate aldolase, or if the activity of the polypeptide B1704, preferably represented by SEQ ID NO. 156543, or a homolog or fragment thereof, or if the activity of a polypeptide encoded by a nucleic acid molecule comprising the nucleic acid SEQ ID NO. 156542, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 156542 or polypeptide SEQ ID NO. 156543, respectively, or a homolog or a fragment thereof, is increased or generated, or if the activity phospho-2-dehydro-3-deoxyheptonate aldolase is increased or generated in a non-human organism, like a microorganism or a plant cell, plant or part thereof, especially with plastidic localization, whereby the respective line disclose in column 7 the fine chemical hentriacontane. For example, an increase of the hentriacontane of at least 1 percent, particularly in a range of 25 to 83-percent is conferred as compared to a corresponding non-transformed wild type non-human organism.

[101977] Accordingly, in one embodiment, an increase of hentriacontane in a non-human organism, as compared to a corresponding non-transformed wild type non-human organism, is conferred in the process of the invention, if the activity of a polypeptide showing the activity of a b1930-protein, or if the activity of the polypeptide B1930, preferably represented by SEQ ID NO. 79182, or a homolog or fragment thereof, or if the activity of a polypeptide encoded by a nucleic acid molecule comprising the nucleic acid SEQ ID NO. 79181, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 79181 or polypeptide SEQ ID NO. 79182, respectively, or a homolog or a fragment thereof, is increased or generated, or if the activity b1930-protein is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 30 to 52-percent is conferred as compared to a corresponding non-transformed wild type non-human organism.

[101978] Accordingly, in one embodiment, an increase of hentriacontane in a non-human organism, as compared to a corresponding non-transformed wild type non-human organism, is conferred in the process of the invention, if the activity of a polypeptide showing the activity of a O-antigen chain length determinant, or if the activity of the polypeptide B2027, preferably represented by SEQ ID NO. 79218, or a homolog or fragment thereof, or if the activity of a polypeptide encoded by a nucleic acid molecule comprising the nucleic acid SEQ ID NO. 79217, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 79217 or polypeptide SEQ ID NO. 79218, respectively, or a homolog or a fragment thereof, is increased or generated, or if the activity O-antigen chain length determinant is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 26 to 43-percent is conferred as compared to a corresponding non-transformed wild type non-human organism.

[101979] Accordingly, in one embodiment, an increase of hentriacontane in a non-human organism, as compared to a corresponding non-transformed wild type non-human organism, is conferred in the process of the invention, if the activity of a polypeptide showing the activity of a GDP-mannose 4,6-dehydratase, or if the activity of the polypeptide B2053, preferably represented by SEQ ID NO. 104278, or a homolog or fragment thereof, or if the activity of a polypeptide encoded by a nucleic acid molecule comprising the nucleic acid SEQ ID NO. 104277, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 104277 or polypeptide SEQ ID NO. 104278, respectively, or a homolog or a fragment thereof, is increased or generated, or if the activity GDP-mannose 4,6-dehydratase is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 24 to 41-percent is conferred as compared to a corresponding non-transformed wild type non-human organism.

[101980] Accordingly, in one embodiment, an increase of hentriacontane in a non-human organism, as compared to a corresponding non-transformed wild type non-human organism, is conferred in the process of the invention, if the activity of a polypeptide showing the activity of a assembly protein for periplasmic nitrate reductase, or if the activity of the polypeptide B2207, preferably represented by SEQ ID NO. 156811, or a homolog or fragment thereof, or if the activity of a polypeptide encoded by a nucleic acid molecule comprising the nucleic acid SEQ ID NO. 156810, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 156810 or polypeptide SEQ ID NO. 156811, respectively, or a homolog or a fragment thereof, is increased or generated, or if the activity assembly protein for periplasmic nitrate reductase is increased or generated in a non-human organism, like a microorganism or a plant cell, plant or part thereof, especially with plastidic localization, whereby the respective line disclose in column 7 the fine chemical hentriacontane. For example, an increase of the hentriacontane of at least 1 percent, particularly in a range of 32 to 43-percent is conferred as compared to a corresponding non-transformed wild type non-human organism.

[101981] Accordingly, in one embodiment, an increase of hentriacontane in a non-human organism, as compared to a corresponding non-transformed wild type non-human organism, is conferred in the process of the invention, if the activity of a polypeptide showing the activity of a ion-transport protein, or if the activity of the polypeptide B2389, preferably represented by SEQ ID NO. 146239, or a homolog or fragment thereof, or if the activity of a polypeptide encoded by a nucleic acid molecule comprising the nucleic acid SEQ ID NO. 146238, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 146238 or polypeptide SEQ ID NO. 146239, respectively, or a homolog or a fragment thereof, is increased or generated, or if the activity ion-transport protein is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 26 to 47-percent is conferred as compared to a corresponding non-transformed wild type non-human organism.

[101982] Accordingly, in one embodiment, an increase of hentriacontane in a non-human organism, as compared to a corresponding non-transformed wild type non-human organism, is conferred in the process of the invention, if the activity of a polypeptide showing the activity of a b2399-protein, or if the activity of the polypeptide B2399, preferably represented by SEQ ID NO. 39238, or a homolog or fragment thereof, or if the activity of a polypeptide encoded by a nucleic acid molecule comprising the nucleic acid SEQ ID NO. 39237, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 39237 or polypeptide SEQ ID NO. 39238, respectively, or a homolog or a fragment thereof, is increased or generated, or if the activity b2399-protein is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 97 to 168-percent is conferred as compared to a corresponding non-transformed wild type non-human organism.

[101983] Accordingly, in one embodiment, an increase of hentriacontane in a non-human organism, as compared to a corresponding non-transformed wild type non-human organism, is conferred in the process of the invention, if the activity of a polypeptide showing the activity of a cysteine synthase A, or if the activity of the polypeptide B2414, preferably represented by SEQ ID NO. 39301, or a homolog or fragment thereof, or if the activity of a polypeptide encoded by a nucleic acid molecule comprising the nucleic acid SEQ ID NO. 39300, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 39300 or polypeptide SEQ ID NO. 39301, respectively, or a homolog or a fragment thereof, is increased or generated, or if the activity cysteine synthase A is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 31 to 77-percent is conferred as compared to a corresponding non-transformed wild type non-human organism.

[101984] Accordingly, in one embodiment, an increase of hentriacontane in a non-human organism, as compared to a corresponding non-transformed wild type non-human organism, is conferred in the process of the invention, if the activity of a polypeptide showing the activity of a ethanolamine utilization protein, or if the activity of the polypeptide B2439, preferably represented by SEQ ID NO. 115629, or a homolog or fragment thereof, or if the activity of a polypeptide encoded by a nucleic acid molecule comprising the nucleic acid SEQ ID NO. 115628, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 115628 or polypeptide SEQ ID NO. 115629, respectively, or a homolog or a fragment thereof, is increased or generated, or if the activity ethanolamine utilization protein is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 28 to 49-percent is conferred as compared to a corresponding non-transformed wild type non-human organism.

[101985] Accordingly, in one embodiment, an increase of hentriacontane in a non-human organism, as compared to a corresponding non-transformed wild type non-human organism, is conferred in the process of the invention, if the activity of a polypeptide showing the activity of a sigma-54 dependent response regulator, or if the activity of the polypeptide B2554, preferably represented by SEQ ID NO. 156911, or a homolog or fragment thereof, or if the activity of a polypeptide encoded by a nucleic acid molecule comprising the nucleic acid SEQ ID NO. 156910, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 156910 or polypeptide SEQ ID NO. 156911, respectively, or a homolog or a fragment thereof, is increased or generated, or if the activity sigma-54 dependent response regulator is increased or generated in a non-human organism, like a microorganism or a plant cell, plant or part thereof, especially with plastidic localization, whereby the respective line disclose in column 7 the fine chemical hentriacontane. For example, an increase of the hentriacontane of at least 1 percent, particularly in a range of 28 to 97-percent is conferred as compared to a corresponding non-transformed wild type non-human organism.

[101986] Accordingly, in one embodiment, an increase of hentriacontane in a non-human organism, as compared to a corresponding non-transformed wild type non-human organism, is conferred in the process of the invention, if the activity of a polypeptide showing the activity of a b2653-protein, or if the activity of the polypeptide B2653, preferably represented by SEQ ID NO. 146289, or a homolog or fragment thereof, or if the activity of a polypeptide encoded by a nucleic acid molecule comprising the nucleic acid SEQ ID NO. 146288, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 146288 or polypeptide SEQ ID NO. 146289, respectively, or a homolog or a fragment thereof, is increased or generated, or if the activity b2653-protein is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 28 to 43-percent is conferred as compared to a corresponding non-transformed wild type non-human organism.

[101987] Accordingly, in one embodiment, an increase of hentriacontane in a non-human organism, as compared to a corresponding non-transformed wild type non-human organism, is conferred in the process of the invention, if the activity of a polypeptide showing the activity of a phosphoadenosine phosphosulfate reductase, or if the activity of the polypeptide B2762, preferably represented by SEQ ID NO. 81452, or a homolog or fragment thereof, or if the activity of a polypeptide encoded by a nucleic acid molecule comprising the nucleic acid SEQ ID NO. 81451, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 81451 or polypeptide SEQ ID NO. 81452, respectively, or a homolog or a fragment thereof, is increased or generated, or if the activity phosphoadenosine phosphosulfate reductase is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 31 to 39-percent is conferred as compared to a corresponding non-transformed wild type non-human organism.

[101988] Accordingly, in one embodiment, an increase of hentriacontane in a non-human organism, as compared to a corresponding non-transformed wild type non-human organism, is conferred in the process of the invention, if the activity of a polypeptide showing the activity of a amino-acid acetyltransferase, or if the activity of the polypeptide B2818, preferably represented by SEQ ID NO. 81577, or a homolog or fragment thereof, or if the activity of a polypeptide encoded by a nucleic acid molecule comprising the nucleic acid SEQ ID NO. 81576, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 81576 or polypeptide SEQ ID NO. 81577, respectively, or a homolog or a fragment thereof, is increased or generated, or if the activity amino-acid acetyltransferase is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 24 to 125-percent is conferred as compared to a corresponding non-transformed wild type non-human organism.

[101989] Accordingly, in one embodiment, an increase of hentriacontane in a non-human organism, as compared to a corresponding non-transformed wild type non-human organism, is conferred in the process of the invention, if the activity of a polypeptide showing the activity of a exonuclease subunit, or if the activity of the polypeptide B2822, preferably represented by SEQ ID NO. 157129, or a homolog or fragment thereof, or if the activity of a polypeptide encoded by a nucleic acid molecule comprising the nucleic acid SEQ ID NO. 157128, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 157128 or polypeptide SEQ ID NO. 157129, respectively, or a homolog or a fragment thereof, is increased or generated, or if the activity exonuclease subunit is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 27 to 47 -percent is conferred as compared to a corresponding non-transformed wild type non-human organism.

[101990] Accordingly, in one embodiment, an increase of hentriacontane in a non-human organism, as compared to a corresponding non-transformed wild type non-human organism, is conferred in the process of the invention, if the activity of a polypeptide showing the activity of a L-asparaginase, or if the activity of the polypeptide B2957, preferably represented by SEQ ID NO. 41500, or a homolog or fragment thereof, or if the activity of a polypeptide encoded by a nucleic acid molecule comprising the nucleic acid SEQ ID NO. 41499, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 41499 or polypeptide SEQ ID NO. 41500, respectively, or a homolog or a fragment thereof, is increased or generated, or if the activity L-asparaginase is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 38 to 65-percent is conferred as compared to a corresponding non-transformed wild type non-human organism.

[101991] Accordingly, in one embodiment, an increase of hentriacontane in a non-human organism, as compared to a corresponding non-transformed wild type non-human organism, is conferred in the process of the invention, if the activity of a polypeptide showing the activity of a ornithine decarboxylase, or if the activity of the polypeptide B2965, preferably represented by SEQ ID NO. 157322, or a homolog or fragment thereof, or if the activity of a polypeptide encoded by a nucleic acid molecule comprising the nucleic acid SEQ ID NO. 157321, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 157321 or polypeptide SEQ ID NO. 157322, respectively, or a homolog or a fragment thereof, is increased or generated, or if the activity ornithine decarboxylase is increased or generated in a non-human organism, like a microorganism or a plant cell, plant or part thereof, especially with plastidic localization, whereby the respective line disclose in column 7 the fine chemical hentriacontane. For example, an increase of the hentriacontane of at least 1 percent, particularly in a range of 37 to 114-percent is conferred as compared to a corresponding non-transformed wild type non-human organism.

[101992] Accordingly, in one embodiment, an increase of hentriacontane in a non-human organism, as compared to a corresponding non-transformed wild type non-human organism, is conferred in the process of the invention, if the activity of a polypeptide showing the activity of a phosphoglycolate phosphatase, or if the activity of the polypeptide B3385, preferably represented by SEQ ID NO. 157451, or a homolog or fragment thereof, or if the activity of a polypeptide encoded by a nucleic acid molecule comprising the nucleic acid SEQ ID NO. 157450, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 157450 or polypeptide SEQ ID NO. 157451, respectively, or a homolog or a fragment thereof, is increased or generated, or if the activity phosphoglycolate phosphatase is increased or generated in a non-human organism, like a microorganism or a plant cell, plant or part thereof, especially with plastidic localization, whereby the respective line disclose in column 7 the fine chemical hentriacontane. For example, an increase of the hentriacontane of at least 1 percent, particularly in a range of 35 to 97-percent is conferred as compared to a corresponding non-transformed wild type non-human organism.

[101993] Accordingly, in one embodiment, an increase of hentriacontane in a non-human organism, as compared to a corresponding non-transformed wild type non-human organism, is conferred in the process of the invention, if the activity of a polypeptide showing the activity of a sugar transport system permease protein, or if the activity of the polypeptide B3568, preferably represented by SEQ ID NO. 125265, or a homolog or fragment thereof, or if the activity of a polypeptide encoded by a nucleic acid molecule comprising the nucleic acid SEQ ID NO. 125264, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 125264 or polypeptide SEQ ID NO. 125265, respectively, or a homolog or a fragment thereof, is increased or generated, or if the activity sugar transport system permease protein is increased or generated in a non-human organism, like a microorganism or a plant cell, plant or part thereof, especially with plastidic localization, whereby the respective line disclose in column 7 the fine chemical hentriacontane. For example, an increase of the hentriacontane of at least 1 percent, particularly in a range of 27 to 47-percent is conferred as compared to a corresponding non-transformed wild type non-human organism.

[101994] Accordingly, in one embodiment, an increase of hentriacontane in a non-human organism, as compared to a corresponding non-transformed wild type non-human organism, is conferred in the process of the invention, if the activity of a polypeptide showing the activity of a cyclin D, or if the activity of the polypeptide GM02LC44512, preferably represented by SEQ ID NO. 46851, or a homolog or fragment thereof, or if the activity of a polypeptide encoded by a nucleic acid molecule comprising the nucleic acid SEQ ID NO. 46850, preferably the coding region thereof, or a homolog or fragment thereof, e.g. derived from Glycine max, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 46850 or polypeptide SEQ ID NO. 46851, respectively, or a homolog or a fragment thereof, is increased or generated, or if the activity cyclin D is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 31 to 89-percent is conferred as compared to a corresponding non-transformed wild type non-human organism.

[101995] Accordingly, in one embodiment, an increase of hentriacontane in a non-human organism, as compared to a corresponding non-transformed wild type non-human organism, is conferred in the process of the invention, if the activity of a polypeptide showing the activity of a short-chain alcohol dehydrogenase family, or if the activity of the polypeptide Slr2124, preferably represented by SEQ ID NO. 59371, or a homolog or fragment thereof, or if the activity of a polypeptide encoded by a nucleic acid molecule comprising the nucleic acid SEQ ID NO. 59370, preferably the coding region thereof, or a homolog or fragment thereof, e.g. derived from Synechocystis sp., is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 59370 or polypeptide SEQ ID NO. 59371, respectively, or a homolog or a fragment thereof, is increased or generated, or if the activity short-chain alcohol dehydrogenase family is increased or generated in a non-human organism, like a microorganism or a plant cell, plant or part thereof, especially with plastidic localization, whereby the respective line disclose in column 7 the fine chemical hentriacontane. For example, an increase of the hentriacontane of at least 1 percent, particularly in a range of 25 to 42-percent is conferred as compared to a corresponding non-transformed wild type non-human organism.

[101996] Accordingly, in one embodiment, an increase of hentriacontane in a non-human organism, as compared to a corresponding non-transformed wild type non-human organism, is conferred in the process of the invention, if the activity of a polypeptide showing the activity of a cell division protein, or if the activity of the polypeptide TTC0035, preferably represented by SEQ ID NO. 60302, or a homolog or fragment thereof, or if the activity of a polypeptide encoded by a nucleic acid molecule comprising the nucleic acid SEQ ID NO. 60301, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 60301 or polypeptide SEQ ID NO. 60302, respectively, or a homolog or a fragment thereof, is increased or generated, or if the activity cell division protein is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 52 to 117-percent is conferred as compared to a corresponding non-transformed wild type non-human organism.

[101997] Accordingly, in one embodiment, an increase of hentriacontane in a non-human organism, as compared to a corresponding non-transformed wild type non-human organism, is conferred in the process of the invention, if the activity of a polypeptide showing the activity of a metal-dependent hydrolase, or if the activity of the polypeptide TTC0917, preferably represented by SEQ ID NO. 61554, or a homolog or fragment thereof, or if the activity of a polypeptide encoded by a nucleic acid molecule comprising the nucleic acid SEQ ID NO. 61553, preferably the coding region thereof, or a homolog or fragment thereof, e.g. derived from Thermus thermophilus, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 61553 or polypeptide SEQ ID NO. 61554, respectively, or a homolog or a fragment thereof, is increased or generated, or if the activity metal-dependent hydrolase is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 25 to 93-percent is conferred as compared to a corresponding non-transformed wild type non-human organism.

[101998] Accordingly, in one embodiment, an increase of hentriacontane in a non-human organism, as compared to a corresponding non-transformed wild type non-human organism, is conferred in the process of the invention, if the activity of a polypeptide showing the activity of a 3-deoxy-7-phosphoheptulonate synthase, or if the activity of the polypeptide Ybr249c, preferably represented by SEQ ID NO. 88880, or a homolog or fragment thereof, or if the activity of a polypeptide encoded by a nucleic acid molecule comprising the nucleic acid SEQ ID NO. 88879, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 88879 or polypeptide SEQ ID NO. 88880, respectively, or a homolog or a fragment thereof, is increased or generated, or if the activity 3-deoxy-7-phosphoheptulonate synthase is increased or generated in a non-human organism, like a microorganism or a plant cell, plant or part thereof, especially with plastidic localization, whereby the respective line disclose in column 7 the fine chemical hentriacontane. For example, an increase of the hentriacontane of at least 1 percent, particularly in a range of 31 to 88-percent is conferred as compared to a corresponding non-transformed wild type non-human organism.

[101999] Accordingly, in one embodiment, an increase of hentriacontane in a non-human organism, as compared to a corresponding non-transformed wild type non-human organism, is conferred in the process of the invention, if the activity of a polypeptide showing the activity of a YFL019C-protein, or if the activity of the polypeptide Yfl019c, preferably represented by SEQ ID NO. 64145, or a homolog or fragment thereof, or if the activity of a polypeptide encoded by a nucleic acid molecule comprising the nucleic acid SEQ ID NO. 64144, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 64144 or polypeptide SEQ ID NO. 64145, respectively, or a homolog or a fragment thereof, is increased or generated, or if the activity YFL019C-protein is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 40 to 1100-percent is conferred as compared to a corresponding non-transformed wild type non-human organism.

[102000] Accordingly, in one embodiment, an increase of hentriacontane in a non-human organism, as compared to a corresponding non-transformed wild type non-human organism, is conferred in the process of the invention, if the activity of a polypeptide showing the activity of a dihydroxyacetone kinase, or if the activity of the polypeptide Yfl053w, preferably represented by SEQ ID NO. 120310, or a homolog or fragment thereof, or if the activity of a polypeptide encoded by a nucleic acid molecule comprising the nucleic acid SEQ ID NO. 120309, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 120309 or polypeptide SEQ ID NO. 120310, respectively, or a homolog or a fragment thereof, is increased or generated, or if the activity dihydroxyacetone kinase is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 25 to 56-percent is conferred as compared to a corresponding non-transformed wild type non-human organism.

[102001] Accordingly, in one embodiment, an increase of hentriacontane in a non-human organism, as compared to a corresponding non-transformed wild type non-human organism, is conferred in the process of the invention, if the activity of a polypeptide showing the activity of a methionine permease, or if the activity of the polypeptide YGRO55W, preferably represented by SEQ ID NO. 157718, or a homolog or fragment thereof, or if the activity of a polypeptide encoded by a nucleic acid molecule comprising the nucleic acid SEQ ID NO. 157717, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 157717 or polypeptide SEQ ID NO. 157718, respectively, or a homolog or a fragment thereof, is increased or generated, or if the activity methionine permease is increased or generated in a non-human organism, like a microorganism or a plant cell, plant or part thereof, especially with plastidic localization, whereby the respective line disclose in column 7 the fine chemical hentriacontane. For example, an increase of the hentriacontane of at least 1 percent, particularly in a range of 26 to 41-percent is conferred as compared to a corresponding non-transformed wild type non-human organism.

[102002] Accordingly, in one embodiment, an increase of hentriacontane in a non-human organism, as compared to a corresponding non-transformed wild type non-human organism, is conferred in the process of the invention, if the activity of a polypeptide showing the activity of a YHL039W-protein, or if the activity of the polypeptide YHL039W, preferably represented by SEQ ID NO. 157833, or a homolog or fragment thereof, or if the activity of a polypeptide encoded by a nucleic acid molecule comprising the nucleic acid SEQ ID NO. 157832, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 157832 or polypeptide SEQ ID NO. 157833, respectively, or a homolog or a fragment thereof, is increased or generated, or if the activity YHL039W-protein is increased or generated in a non-human organism, like a microorganism or a plant cell, plant or part thereof, especially with plastidic localization, whereby the respective line disclose in column 7 the fine chemical hentriacontane. For example, an increase of the hentriacontane of at least 1 percent, particularly in a range of 25 to 53-percent is conferred as compared to a corresponding non-transformed wild type non-human organism.

[102003] Accordingly, in one embodiment, an increase of hentriacontane in a non-human organism, as compared to a corresponding non-transformed wild type non-human organism, is conferred in the process of the invention, if the activity of a polypeptide showing the activity of a YHR022C-A-protein, or if the activity of the polypeptide Yhr022c-a, preferably represented by SEQ ID NO. 157859, or a homolog or fragment thereof, or if the activity of a polypeptide encoded by a nucleic acid molecule comprising the nucleic acid SEQ ID NO. 157858, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 157858 or polypeptide SEQ ID NO. 157859, respectively, or a homolog or a fragment thereof, is increased or generated, or if the activity YHR022C-A-protein is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 25 to 111-percent is conferred as compared to a corresponding non-transformed wild type non-human organism.

[102004] Accordingly, in one embodiment, an increase of hentriacontane in a non-human organism, as compared to a corresponding non-transformed wild type non-human organism, is conferred in the process of the invention, if the activity of a polypeptide showing the activity of a YJL127W-A-protein, or if the activity of the polypeptide Yjl127w-a, preferably represented by SEQ ID NO. 97632, or a homolog or fragment thereof, or if the activity of a polypeptide encoded by a nucleic acid molecule comprising the nucleic acid SEQ ID NO. 97631, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 97631 or polypeptide SEQ ID NO. 97632, respectively, or a homolog or a fragment thereof, is increased or generated, or if the activity YJL127W-A-protein is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 36 to 78-percent is conferred as compared to a corresponding non-transformed wild type non-human organism.

[102005] Accordingly, in one embodiment, an increase of hentriacontane in a non-human organism, as compared to a corresponding non-transformed wild type non-human organism, is conferred in the process of the invention, if the activity of a polypeptide showing the activity of a ylr157c-protein, or if the activity of the polypeptide Ylr157c, preferably represented by SEQ ID NO. 157863, or a homolog or fragment thereof, or if the activity of a polypeptide encoded by a nucleic acid molecule comprising the nucleic acid SEQ ID NO. 157862, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 157862 or polypeptide SEQ ID NO. 157863, respectively, or a homolog or a fragment thereof, is increased or generated, or if the activity ylr157c-protein is increased or generated in a non-human organism, like a microorganism or a plant cell, plant or part thereof, especially with plastidic localization, whereby the respective line disclose in column 7 the fine chemical hentriacontane. For example, an increase of the hentriacontane of at least 1 percent, particularly in a range of 26 to 45-percent is conferred as compared to a corresponding non-transformed wild type non-human organism.

[102006] Accordingly, in one embodiment, an increase of hentriacontane in a non-human organism, as compared to a corresponding non-transformed wild type non-human organism, is conferred in the process of the invention, if the activity of a polypeptide showing the activity of a ynl321w-protein, or if the activity of the polypeptide Ynl321w, preferably represented by SEQ ID NO. 129547, or a homolog or fragment thereof, or if the activity of a polypeptide encoded by a nucleic acid molecule comprising the nucleic acid SEQ ID NO. 129546, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 129546 or polypeptide SEQ ID NO. 129547, respectively, or a homolog or a fragment thereof, is increased or generated, or if the activity ynl321w-protein is increased or generated in a non-human organism, like a microorganism or a plant cell, plant or part thereof, especially with plastidic localization, whereby the respective line disclose in column 7 the fine chemical hentriacontane. For example, an increase of the hentriacontane of at least 1 percent, particularly in a range of 25 to 57-percent is conferred as compared to a corresponding non-transformed wild type non-human organism.

[102007] Accordingly, in one embodiment, an increase of hentriacontane in a non-human organism, as compared to a corresponding non-transformed wild type non-human organism, is conferred in the process of the invention, if the activity of a polypeptide showing the activity of a ypr170c-protein, or if the activity of the polypeptide Ypr170c, preferably represented by SEQ ID NO. 113648, or a homolog or fragment thereof, or if the activity of a polypeptide encoded by a nucleic acid molecule comprising the nucleic acid SEQ ID NO. 113647, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 113647 or polypeptide SEQ ID NO. 113648, respectively, or a homolog or a fragment thereof, is increased or generated, or if the activity ypr170c-protein is increased or generated in a non-human organism, like a microorganism or a plant cell, plant or part thereof, especially with plastidic localization, whereby the respective line disclose in column 7 the fine chemical hentriacontane. For example, an increase of the hentriacontane of at least 1 percent, particularly in a range of 32 to 72-percent is conferred as compared to a corresponding non-transformed wild type non-human organism.

[102008] Accordingly, in one embodiment, an increase of hentriacontane in a non-human organism, as compared to a corresponding non-transformed wild type non-human organism, is conferred in the process of the invention, if the activity of a polypeptide showing the activity of a YPR172W-protein, or if the activity of the polypeptide YPR172W, preferably represented by SEQ ID NO. 158034, or a homolog or fragment thereof, or if the activity of a polypeptide encoded by a nucleic acid molecule comprising the nucleic acid SEQ ID NO. 158033, preferably the coding region thereof, or a homolog or fragment thereof, e.g. derived from Saccharomyces cerevisiae, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 158033 or polypeptide SEQ ID NO. 158034, respectively, or a homolog or a fragment thereof, is increased or generated, or if the activity YPR172W-protein is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 26 to 72-percent is conferred as compared to a corresponding non-transformed wild type non-human organism.

[102009] Accordingly, in one embodiment, an increase of hentriacontane in a non-human organism, as compared to a corresponding non-transformed wild type non-human organism, is conferred in the process of the invention, if the activity of a polypeptide showing the activity of a b3644-protein, or if the activity of the polypeptide B3644, preferably represented by SEQ ID NO. 125349, or a homolog or fragment thereof, or if the activity of a polypeptide encoded by a nucleic acid molecule comprising the nucleic acid SEQ ID NO. 125348, preferably the coding region thereof, or a homolog or fragment thereof, e.g. derived from Escherichia coli, is increased or generated. For example the activity of a nucleic acid molecule or a polypeptide comprising the nucleic acid, preferably the coding region thereof, or polypeptide or the consensus sequence or the polypeptide motif, as depicted in Table I, II or IV, application No. 20, column 5 or 8 in the respective same line as the nucleic acid molecule SEQ ID NO. 125348 or polypeptide SEQ ID NO. 125349, respectively, or a homolog or a fragment thereof, is increased or generated, or if the activity b3644-protein is increased or generated in a non-human organism, like a microorganism or a plant cell, plant or part thereof, especially with non-targeted localization, whereby the respective line disclose in column 7 the fine chemical hentriacontane. For example, an increase of the hentriacontane of at least 1 percent, particularly in a range of 8 to 450-percent is conferred as compared to a corresponding non-transformed wild type non-human organism.

[102010] [0096.1.7.20] to [0103.1.7.20] for the disclosure of these paragraphs see [0096.1.7.7] to [0103.1.7.7] above

[102011] In this paragraph “A” means “Goffeau et al., Science 274 (5287), 546 (1996)”, “B” means “Blattner et al., Science 277 (5331), 1453 (1997)”, “C” means “Nakamura et al., Nucleic Acids Res. 28(1), 72 (2000)”, “D” means “deposition of the sequences to the EMBL/GenBank/DDBJ databases under “Sequencing of the draft genome assembly of Azotobacter vinelandii AvOP”. in June 2005 by Copeland et al.”, “E” means “Henne et al., Nat. Biotechnol. 22 (5), 547 (2004)”, “F” means “Kaul et al., Nature 408 (6814), 796 (2000)”, and “G” means “Matsumoto et al., Nature 436 (7052), 793 (2005)”.

[102012] The nucleic acid sequence of At2g36580 from Arabidopsis thaliana, e.g. as shown in the respective line in column 5 of Table I, application no. 20, has been published in F. And the activity of the gene product thereof is the activity of pyruvate kinase.

[102013] Accordingly, in one embodiment, the process of the present invention for producing hentriacontane in a non-human organism, like a microorganism or a plant or a part thereof, comprises increasing or generating the activity of a gene product with the activity of a gene product conferring the activity of “pyruvate kinase”, especially from Arabidopsis thaliana or its functional equivalent or its homolog, e.g. the increase of
[102014] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said At2g36580, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said At2g36580, and preferably the activity is increased plastidic, or
[102015] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said At2g36580, or a functional equivalent or a homolog thereof as depicted in column 8 of Table II, application no. 20, preferably a homolog or functional equivalent as depicted in column 8 of Table II B, application no. 20, and being depicted in the same respective line as said At2g36580, and preferably the activity is increased plastidic, whereby the respective line disclose in column 7 the fine chemical hentriacontane.

[102016] Accordingly, in one embodiment, the molecule which activity is to be increased in the process of the invention is the gene product with an activity as a “pyruvate kinase”, preferably it is the molecule of section (a) or (b) of this paragraph.

[102017] In particular, it was observed that in plants, especially in Arabidopsis thaliana, increasing or generating the activity of a gene product plastidic with the activity of a “pyruvate kinase”, preferably being encoded by a gene comprising the nucleic acid sequence SEQ ID NO. 113714, preferably the coding region thereof, conferred the production of or the increase in hentriacontane compared with the wild type control.

[102018] The nucleic acid sequence of At2g42830 from Arabidopsis thaliana, e.g. as shown in the respective line in column 5 of Table I, application no. 20, has been published in F. And the activity of the gene product thereof is the activity of MADS box protein transcription factor.

[102019] Accordingly, in one embodiment, the process of the present invention for producing hentriacontane in a non-human organism, like a microorganism or a plant or a part thereof, comprises increasing or generating the activity of a gene product with the activity of a gene product conferring the activity of “MADS box protein transcription factor”, especially from Arabidopsis thaliana or its functional equivalent or its homolog, e.g. the increase of
[102020] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said At2g42830, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said At2g42830, and preferably the activity is increased non-targeted, or
[102021] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said At2g42830, or a functional equivalent or a homolog thereof as depicted in column 8 of Table II, application no. 20, preferably a homolog or functional equivalent as depicted in column 8 of Table II B, application no. 20, and being depicted in the same respective line as said At2g42830, and preferably the activity is increased non-targeted, whereby the respective line disclose in column 7 the fine chemical hentriacontane.

[102022] Accordingly, in one embodiment, the molecule which activity is to be increased in the process of the invention is the gene product with an activity as a “MADS box protein transcription factor”, preferably it is the molecule of section (a) or (b) of this paragraph.

[102023] In particular, it was observed that in plants, especially in Arabidopsis thaliana, increasing or generating the activity of a gene product non-targeted with the activity of a “MADS box protein transcription factor”, preferably being encoded by a gene comprising the nucleic acid sequence SEQ ID NO. 108727, preferably the coding region thereof, conferred the production of or the increase in hentriacontane compared with the wild type control.

[102024] The nucleic acid sequence of At2g45420 from Arabidopsis thaliana, e.g. as shown in the respective line in column 5 of Table I, application no. 20, has been published in F. And the activity of the gene product thereof is the activity of At2g45420-protein.

[102025] Accordingly, in one embodiment, the process of the present invention for producing hentriacontane in a non-human organism, like a microorganism or a plant or a part thereof, comprises increasing or generating the activity of a gene product with the activity of a gene product conferring the activity of “At2g45420-protein”, especially from Arabidopsis thaliana or its functional equivalent or its homolog, e.g. the increase of
[102026] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said At2g45420, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said At2g45420, and preferably the activity is increased non-targeted, or
[102027] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said At2g45420, or a functional equivalent or a homolog thereof as depicted in column 8 of Table II, application no. 20, preferably a homolog or functional equivalent as depicted in column 8 of Table II B, application no. 20, and being depicted in the same respective line as said At2g45420, and preferably the activity is increased non-targeted, whereby the respective line disclose in column 7 the fine chemical hentriacontane.

[102028] Accordingly, in one embodiment, the molecule which activity is to be increased in the process of the invention is the gene product with an activity as a “At2g45420-protein”, preferably it is the molecule of section (a) or (b) of this paragraph.

[102029] In particular, it was observed that in plants, especially in Arabidopsis thaliana, increasing or generating the activity of a gene product non-targeted with the activity of a “At2g45420-protein”, preferably being encoded by a gene comprising the nucleic acid sequence SEQ ID NO. 1623, preferably the coding region thereof, conferred the production of or the increase in hentriacontane compared with the wild type control.

[102030] The nucleic acid sequence of At4g09960 from Arabidopsis thaliana, e.g. as shown in the respective line in column 5 of Table I, application no. 20, has been published in F. And the activity of the gene product thereof is the activity of transcription factor.

[102031] Accordingly, in one embodiment, the process of the present invention for producing hentriacontane in a non-human organism, like a microorganism or a plant or a part thereof, comprises increasing or generating the activity of a gene product with the activity of a gene product conferring the activity of “transcription factor”, especially from Arabidopsis thaliana or its functional equivalent or its homolog, e.g. the increase of
[102032] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said At4g09960, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said At4g09960, and preferably the activity is increased non-targeted, or
[102033] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said At4g09960, or a functional equivalent or a homolog thereof as depicted in column 8 of Table II, application no. 20, preferably a homolog or functional equivalent as depicted in column 8 of Table II B, application no. 20, and being depicted in the same respective line as said At4g09960, and preferably the activity is increased non-targeted, whereby the respective line disclose in column 7 the fine chemical hentriacontane.

[102034] Accordingly, in one embodiment, the molecule which activity is to be increased in the process of the invention is the gene product with an activity as a “transcription factor”, preferably it is the molecule of section (a) or (b) of this paragraph.

[102035] In particular, it was observed that in plants, especially in Arabidopsis thaliana, increasing or generating the activity of a gene product non-targeted with the activity of a “transcription factor”, preferably being encoded by a gene comprising the nucleic acid sequence SEQ ID NO. 109717, preferably the coding region thereof, conferred the production of or the increase in hentriacontane compared with the wild type control.

[102036] The nucleic acid sequence of At5g43630 from Arabidopsis thaliana, e.g. as shown in the respective line in column 5 of Table I, application no. 20, has been published in F. And the activity of the gene product thereof is the activity of At5g43630-protein.

[102037] Accordingly, in one embodiment, the process of the present invention for producing hentriacontane in a non-human organism, like a microorganism or a plant or a part thereof, comprises increasing or generating the activity of a gene product with the activity of a gene product conferring the activity of “At5g43630-protein”, especially from Arabidopsis thaliana or its functional equivalent or its homolog, e.g. the increase of
[102038] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said At5g43630, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said At5g43630, and preferably the activity is increased non-targeted, or
[102039] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said At5g43630, or a functional equivalent or a homolog thereof as depicted in column 8 of Table II, application no. 20, preferably a homolog or functional equivalent as depicted in column 8 of Table II B, application no. 20, and being depicted in the same respective line as said At5g43630, and preferably the activity is increased non-targeted, whereby the respective line disclose in column 7 the fine chemical hentriacontane.

[102040] Accordingly, in one embodiment, the molecule which activity is to be increased in the process of the invention is the gene product with an activity as a “At5g43630-protein”, preferably it is the molecule of section (a) or (b) of this paragraph.

[102041] In particular, it was observed that in plants, especially in Arabidopsis thaliana, increasing or generating the activity of a gene product non-targeted with the activity of a “At5g43630-protein”, preferably being encoded by a gene comprising the nucleic acid sequence SEQ ID NO. 142929, preferably the coding region thereof, conferred the production of or the increase in hentriacontane compared with the wild type control.

[102042] The nucleic acid sequence of At5g60110 from Arabidopsis thaliana, e.g. as shown in the respective line in column 5 of Table I, application no. 20, has been published in F. And the activity of the gene product thereof is the activity of RNA-binding protein.

[102043] Accordingly, in one embodiment, the process of the present invention for producing hentriacontane in a non-human organism, like a microorganism or a plant or a part thereof, comprises increasing or generating the activity of a gene product with the activity of a gene product conferring the activity of “RNA-binding protein”, especially from Arabidopsis thaliana or its functional equivalent or its homolog, e.g. the increase of
[102044] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said At5g60110, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said At5g60110, and preferably the activity is increased non-targeted, or
[102045] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said At5g60110, or a functional equivalent or a homolog thereof as depicted in column 8 of Table II, application no. 20, preferably a homolog or functional equivalent as depicted in column 8 of Table II B, application no. 20, and being depicted in the same respective line as said At5g60110, and preferably the activity is increased non-targeted, whereby the respective line disclose in column 7 the fine chemical hentriacontane.

[102046] Accordingly, in one embodiment, the molecule which activity is to be increased in the process of the invention is the gene product with an activity as a “RNA-binding protein”, preferably it is the molecule of section (a) or (b) of this paragraph.

[102047] In particular, it was observed that in plants, especially in Arabidopsis thaliana, increasing or generating the activity of a gene product non-targeted with the activity of a “RNA-binding protein”, preferably being encoded by a gene comprising the nucleic acid sequence SEQ ID NO. 70006, preferably the coding region thereof, conferred the production of or the increase in hentriacontane compared with the wild type control.

[102048] The nucleic acid sequence of AvinDRAFT_{—}3209 from Azotobacter vinelandii, e.g. as shown in the respective line in column 5 of Table I, application no. 20, has been published in D. And the activity of the gene product thereof is the activity of transcriptional regulator.

[102049] Accordingly, in one embodiment, the process of the present invention for producing hentriacontane in a non-human organism, like a microorganism or a plant or a part thereof, comprises increasing or generating the activity of a gene product with the activity of a gene product conferring the activity of “transcriptional regulator”, especially from Azotobacter vinelandii or its functional equivalent or its homolog, e.g. the increase of
[102050] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said AvinDRAFT_{—}3209, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said AvinDRAFT_{—}3209, and preferably the activity is increased non-targeted, or
[102051] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said AvinDRAFT_{—}3209, or a functional equivalent or a homolog thereof as depicted in column 8 of Table II, application no. 20, preferably a homolog or functional equivalent as depicted in column 8 of Table II B, application no. 20, and being depicted in the same respective line as said AvinDRAFT_{—}3209, and preferably the activity is increased non-targeted, whereby the respective line disclose in column 7 the fine chemical hentriacontane.

[102052] Accordingly, in one embodiment, the molecule which activity is to be increased in the process of the invention is the gene product with an activity as a “transcriptional regulator”, preferably it is the molecule of section (a) or (b) of this paragraph.

[102053] In particular, it was observed that in plants, especially in Arabidopsis thaliana, increasing or generating the activity of a gene product non-targeted with the activity of a “transcriptional regulator”, preferably being encoded by a gene comprising the nucleic acid sequence SEQ ID NO. 29286, preferably the coding region thereof, conferred the production of or the increase in hentriacontane compared with the wild type control.

[102054] The nucleic acid sequence of AvinDRAFT_{—}5292 from Azotobacter vinelandii, e.g. as shown in the respective line in column 5 of Table I, application no. 20, has been published in D. And the activity of the gene product thereof is the activity of fumarylacetoacetate hydrolase.

[102055] Accordingly, in one embodiment, the process of the present invention for producing hentriacontane in a non-human organism, like a microorganism or a plant or a part thereof, comprises increasing or generating the activity of a gene product with the activity of a gene product conferring the activity of “fumarylacetoacetate hydrolase”, especially from Azotobacter vinelandii or its functional equivalent or its homolog, e.g. the increase of
[102056] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said AvinDRAFT_{—}5292, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said AvinDRAFT_{—}5292, and preferably the activity is increased non-targeted, or
[102057] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said AvinDRAFT_{—}5292, or a functional equivalent or a homolog thereof as depicted in column 8 of Table II, application no. 20, preferably a homolog or functional equivalent as depicted in column 8 of Table II B, application no. 20, and being depicted in the same respective line as said AvinDRAFT_{—}5292, and preferably the activity is increased non-targeted, whereby the respective line disclose in column 7 the fine chemical hentriacontane.

[102058] Accordingly, in one embodiment, the molecule which activity is to be increased in the process of the invention is the gene product with an activity as a “fumarylacetoacetate hydrolase”, preferably it is the molecule of section (a) or (b) of this paragraph.

[102059] In particular, it was observed that in plants, especially in Arabidopsis thaliana, increasing or generating the activity of a gene product non-targeted with the activity of a “fumarylacetoacetate hydrolase”, preferably being encoded by a gene comprising the nucleic acid sequence SEQ ID NO. 32308, preferably the coding region thereof, conferred the production of or the increase in hentriacontane compared with the wild type control.

[102060] The nucleic acid sequence of B0121 from Escherichia coli, e.g. as shown in the respective line in column 5 of Table I, application no. 20, has been published in B. And the activity of the gene product thereof is the activity of spermidine synthase.

[102061] Accordingly, in one embodiment, the process of the present invention for producing hentriacontane in a non-human organism, like a microorganism or a plant or a part thereof, comprises increasing or generating the activity of a gene product with the activity of a gene product conferring the activity of “spermidine synthase”, especially from Escherichia coli or its functional equivalent or its homolog, e.g. the increase of
[102062] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B0121, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B0121, and preferably the activity is increased plastidic, or
[102063] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B0121, or a functional equivalent or a homolog thereof as depicted in column 8 of Table II, application no. 20, preferably a homolog or functional equivalent as depicted in column 8 of Table II B, application no. 20, and being depicted in the same respective line as said B0121, and preferably the activity is increased plastidic, whereby the respective line disclose in column 7 the fine chemical hentriacontane.

[102064] Accordingly, in one embodiment, the molecule which activity is to be increased in the process of the invention is the gene product with an activity as a “spermidine synthase”, preferably it is the molecule of section (a) or (b) of this paragraph.

[102065] In particular, it was observed that in plants, especially in Arabidopsis thaliana, increasing or generating the activity of a gene product plastidic with the activity of a “spermidine synthase”, preferably being encoded by a gene comprising the nucleic acid sequence SEQ ID NO. 154509, preferably the coding region thereof, conferred the production of or the increase in hentriacontane compared with the wild type control.

[102066] The nucleic acid sequence of B0161 from Escherichia coli, e.g. as shown in the respective line in column 5 of Table I, application no. 20, has been published in B. And the activity of the gene product thereof is the activity of serine protease.

[102067] Accordingly, in one embodiment, the process of the present invention for producing hentriacontane in a non-human organism, like a microorganism or a plant or a part thereof, comprises increasing or generating the activity of a gene product with the activity of a gene product conferring the activity of “serine protease”, especially from Escherichia coli or its functional equivalent or its homolog, e.g. the increase of
[102068] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B0161, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B0161, and preferably the activity is increased non-targeted, or
[102069] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B0161, or a functional equivalent or a homolog thereof as depicted in column 8 of Table II, application no. 20, preferably a homolog or functional equivalent as depicted in column 8 of Table II B, application no. 20, and being depicted in the same respective line as said B0161, and preferably the activity is increased non-targeted, whereby the respective line disclose in column 7 the fine chemical hentriacontane.

[102070] Accordingly, in one embodiment, the molecule which activity is to be increased in the process of the invention is the gene product with an activity as a “serine protease”, preferably it is the molecule of section (a) or (b) of this paragraph.

[102071] In particular, it was observed that in plants, especially in Arabidopsis thaliana, increasing or generating the activity of a gene product non-targeted with the activity of a “serine protease”, preferably being encoded by a gene comprising the nucleic acid sequence SEQ ID NO. 7081, preferably the coding region thereof, conferred the production of or the increase in hentriacontane compared with the wild type control.

[102072] The nucleic acid sequence of B0236 from Escherichia coli, e.g. as shown in the respective line in column 5 of Table I, application no. 20, has been published in B. And the activity of the gene product thereof is the activity of peptide chain release factor.

[102073] Accordingly, in one embodiment, the process of the present invention for producing hentriacontane in a non-human organism, like a microorganism or a plant or a part thereof, comprises increasing or generating the activity of a gene product with the activity of a gene product conferring the activity of “peptide chain release factor”, especially from Escherichia coli or its functional equivalent or its homolog, e.g. the increase of
[102074] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B0236, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B0236, and preferably the activity is increased non-targeted, or
[102075] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B0236, or a functional equivalent or a homolog thereof as depicted in column 8 of Table II, application no. 20, preferably a homolog or functional equivalent as depicted in column 8 of Table II B, application no. 20, and being depicted in the same respective line as said B0236, and preferably the activity is increased non-targeted, whereby the respective line disclose in column 7 the fine chemical hentriacontane.

[102076] Accordingly, in one embodiment, the molecule which activity is to be increased in the process of the invention is the gene product with an activity as a “peptide chain release factor”, preferably it is the molecule of section (a) or (b) of this paragraph.

[102077] In particular, it was observed that in plants, especially in Arabidopsis thaliana, increasing or generating the activity of a gene product non-targeted with the activity of a “peptide chain release factor”, preferably being encoded by a gene comprising the nucleic acid sequence SEQ ID NO. 155230, preferably the coding region thereof, conferred the production of or the increase in hentriacontane compared with the wild type control.

[102078] The nucleic acid sequence of B0577 from Escherichia coli, e.g. as shown in the respective line in column 5 of Table I, application no. 20, has been published in B. And the activity of the gene product thereof is the activity of mechanosensitive ion channel.

[102079] Accordingly, in one embodiment, the process of the present invention for producing hentriacontane in a non-human organism, like a microorganism or a plant or a part thereof, comprises increasing or generating the activity of a gene product with the activity of a gene product conferring the activity of “mechanosensitive ion channel”, especially from Escherichia coli or its functional equivalent or its homolog, e.g. the increase of
[102080] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B0577, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B0577, and preferably the activity is increased non-targeted, or
[102081] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B0577, or a functional equivalent or a homolog thereof as depicted in column 8 of Table II, application no. 20, preferably a homolog or functional equivalent as depicted in column 8 of Table II B, application no. 20, and being depicted in the same respective line as said B0577, and preferably the activity is increased non-targeted, whereby the respective line disclose in column 7 the fine chemical hentriacontane.

[102082] Accordingly, in one embodiment, the molecule which activity is to be increased in the process of the invention is the gene product with an activity as a “mechanosensitive ion channel”, preferably it is the molecule of section (a) or (b) of this paragraph.

[102083] In particular, it was observed that in plants, especially in Arabidopsis thaliana, increasing or generating the activity of a gene product non-targeted with the activity of a “mechanosensitive ion channel”, preferably being encoded by a gene comprising the nucleic acid sequence SEQ ID NO. 155370, preferably the coding region thereof, conferred the production of or the increase in hentriacontane compared with the wild type control.

[102084] The nucleic acid sequence of B0651 from Escherichia coli, e.g. as shown in the respective line in column 5 of Table I, application no. 20, has been published in B. And the activity of the gene product thereof is the activity of pyrimidine-specific ribonucleoside hydrolase.

[102085] Accordingly, in one embodiment, the process of the present invention for producing hentriacontane in a non-human organism, like a microorganism or a plant or a part thereof, comprises increasing or generating the activity of a gene product with the activity of a gene product conferring the activity of “pyrimidine-specific ribonucleoside hydrolase”, especially from Escherichia coli or its functional equivalent or its homolog, e.g. the increase of
[102086] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B0651, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B0651, and preferably the activity is increased non-targeted, or
[102087] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B0651, or a functional equivalent or a homolog thereof as depicted in column 8 of Table II, application no. 20, preferably a homolog or functional equivalent as depicted in column 8 of Table II B, application no. 20, and being depicted in the same respective line as said B0651, and preferably the activity is increased non-targeted, whereby the respective line disclose in column 7 the fine chemical hentriacontane.

[102088] Accordingly, in one embodiment, the molecule which activity is to be increased in the process of the invention is the gene product with an activity as a “pyrimidine-specific ribonucleoside hydrolase”, preferably it is the molecule of section (a) or (b) of this paragraph.

[102089] In particular, it was observed that in plants, especially in Arabidopsis thaliana, increasing or generating the activity of a gene product non-targeted with the activity of a “pyrimidine-specific ribonucleoside hydrolase”, preferably being encoded by a gene comprising the nucleic acid sequence SEQ ID NO. 143954, preferably the coding region thereof, conferred the production of or the increase in hentriacontane compared with the wild type control.

[102090] The nucleic acid sequence of B0787 from Escherichia coli, e.g. as shown in the respective line in column 5 of Table I, application no. 20, has been published in B. And the activity of the gene product thereof is the activity of inner membrane protein.

[102091] Accordingly, in one embodiment, the process of the present invention for producing hentriacontane in a non-human organism, like a microorganism or a plant or a part thereof, comprises increasing or generating the activity of a gene product with the activity of a gene product conferring the activity of “inner membrane protein”, especially from Escherichia coli or its functional equivalent or its homolog, e.g. the increase of
[102092] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B0787, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B0787, and preferably the activity is increased non-targeted, or
[102093] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B0787, or a functional equivalent or a homolog thereof as depicted in column 8 of Table II, application no. 20, preferably a homolog or functional equivalent as depicted in column 8 of Table II B, application no. 20, and being depicted in the same respective line as said B0787, and preferably the activity is increased non-targeted, whereby the respective line disclose in column 7 the fine chemical hentriacontane.

[102094] Accordingly, in one embodiment, the molecule which activity is to be increased in the process of the invention is the gene product with an activity as a “inner membrane protein”, preferably it is the molecule of section (a) or (b) of this paragraph.

[102095] In particular, it was observed that in plants, especially in Arabidopsis thaliana, increasing or generating the activity of a gene product non-targeted with the activity of a “inner membrane protein”, preferably being encoded by a gene comprising the nucleic acid sequence SEQ ID NO. 155613, preferably the coding region thereof, conferred the production of or the increase in hentriacontane compared with the wild type control.

[102096] The nucleic acid sequence of B0833 from Escherichia coli, e.g. as shown in the respective line in column 5 of Table I, application no. 20, has been published in B. And the activity of the gene product thereof is the activity of b0833-protein.

[102097] Accordingly, in one embodiment, the process of the present invention for producing hentriacontane in a non-human organism, like a microorganism or a plant or a part thereof, comprises increasing or generating the activity of a gene product with the activity of a gene product conferring the activity of “b0833-protein”, especially from Escherichia coli or its functional equivalent or its homolog, e.g. the increase of
[102098] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B0833, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B0833, and preferably the activity is increased non-targeted, or
[102099] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B0833, or a functional equivalent or a homolog thereof as depicted in column 8 of Table II, application no. 20, preferably a homolog or functional equivalent as depicted in column 8 of Table II B, application no. 20, and being depicted in the same respective line as said B0833, and preferably the activity is increased non-targeted, whereby the respective line disclose in column 7 the fine chemical hentriacontane.

[102100] Accordingly, in one embodiment, the molecule which activity is to be increased in the process of the invention is the gene product with an activity as a “b0833-protein”, preferably it is the molecule of section (a) or (b) of this paragraph.

[102101] In particular, it was observed that in plants, especially in Arabidopsis thaliana, increasing or generating the activity of a gene product non-targeted with the activity of a “b0833-protein”, preferably being encoded by a gene comprising the nucleic acid sequence SEQ ID NO. 155657, preferably the coding region thereof, conferred the production of or the increase in hentriacontane compared with the wild type control.

[102102] The nucleic acid sequence of B0921 from Escherichia coli, e.g. as shown in the respective line in column 5 of Table I, application no. 20, has been published in B. And the activity of the gene product thereof is the activity of S-adenosyl-L-methionine-dependent methyltransferase.

[102103] Accordingly, in one embodiment, the process of the present invention for producing hentriacontane in a non-human organism, like a microorganism or a plant or a part thereof, comprises increasing or generating the activity of a gene product with the activity of a gene product conferring the activity of “S-adenosyl-L-methionine-dependent methyltransferase”, especially from Escherichia coli or its functional equivalent or its homolog, e.g. the increase of
[102104] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B0921, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B0921, and preferably the activity is increased plastidic, or
[102105] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B0921, or a functional equivalent or a homolog thereof as depicted in column 8 of Table II, application no. 20, preferably a homolog or functional equivalent as depicted in column 8 of Table II B, application no. 20, and being depicted in the same respective line as said B0921, and preferably the activity is increased plastidic, whereby the respective line disclose in column 7 the fine chemical hentriacontane.

[102106] Accordingly, in one embodiment, the molecule which activity is to be increased in the process of the invention is the gene product with an activity as a “S-adenosyl-L-methionine-dependent methyltransferase”, preferably it is the molecule of section (a) or (b) of this paragraph.

[102107] In particular, it was observed that in plants, especially in Arabidopsis thaliana, increasing or generating the activity of a gene product plastidic with the activity of a “S-adenosyl-L-methionine-dependent methyltransferase”, preferably being encoded by a gene comprising the nucleic acid sequence SEQ ID NO. 155665, preferably the coding region thereof, conferred the production of or the increase in hentriacontane compared with the wild type control.

[102108] The nucleic acid sequence of B1259 from Escherichia coli, e.g. as shown in the respective line in column 5 of Table I, application no. 20, has been published in B. And the activity of the gene product thereof is the activity of b1259-protein.

[102109] Accordingly, in one embodiment, the process of the present invention for producing hentriacontane in a non-human organism, like a microorganism or a plant or a part thereof, comprises increasing or generating the activity of a gene product with the activity of a gene product conferring the activity of “b1259-protein”, especially from Escherichia coli or its functional equivalent or its homolog, e.g. the increase of
[102110] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B1259, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B1259, and preferably the activity is increased non-targeted, or
[102111] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B1259, or a functional equivalent or a homolog thereof as depicted in column 8 of Table II, application no. 20, preferably a homolog or functional equivalent as depicted in column 8 of Table II B, application no. 20, and being depicted in the same respective line as said B1259, and preferably the activity is increased non-targeted, whereby the respective line disclose in column 7 the fine chemical hentriacontane.

[102112] Accordingly, in one embodiment, the molecule which activity is to be increased in the process of the invention is the gene product with an activity as a “b1259-protein”, preferably it is the molecule of section (a) or (b) of this paragraph.

[102113] In particular, it was observed that in plants, especially in Arabidopsis thaliana, increasing or generating the activity of a gene product non-targeted with the activity of a “b1259-protein”, preferably being encoded by a gene comprising the nucleic acid sequence SEQ ID NO. 37503, preferably the coding region thereof, conferred the production of or the increase in hentriacontane compared with the wild type control.

[102114] The nucleic acid sequence of B1281 from Escherichia coli, e.g. as shown in the respective line in column 5 of Table I, application no. 20, has been published in B. And the activity of the gene product thereof is the activity of orotidine-5′-phosphate decarboxylase.

[102115] Accordingly, in one embodiment, the process of the present invention for producing hentriacontane in a non-human organism, like a microorganism or a plant or a part thereof, comprises increasing or generating the activity of a gene product with the activity of a gene product conferring the activity of “orotidine-5′-phosphate decarboxylase”, especially from Escherichia coli or its functional equivalent or its homolog, e.g. the increase of
[102116] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B1281, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B1281, and preferably the activity is increased plastidic, or
[102117] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B1281, or a functional equivalent or a homolog thereof as depicted in column 8 of Table II, application no. 20, preferably a homolog or functional equivalent as depicted in column 8 of Table II B, application no. 20, and being depicted in the same respective line as said B1281, and preferably the activity is increased plastidic, whereby the respective line disclose in column 7 the fine chemical hentriacontane.

[102118] Accordingly, in one embodiment, the molecule which activity is to be increased in the process of the invention is the gene product with an activity as a “orotidine-5′-phosphate decarboxylase”, preferably it is the molecule of section (a) or (b) of this paragraph.

[102119] In particular, it was observed that in plants, especially in Arabidopsis thaliana, increasing or generating the activity of a gene product plastidic with the activity of a “orotidine-5′-phosphate decarboxylase”, preferably being encoded by a gene comprising the nucleic acid sequence SEQ ID NO. 155890, preferably the coding region thereof, conferred the production of or the increase in hentriacontane compared with the wild type control.

[102120] The nucleic acid sequence of B1343 from Escherichia coli, e.g. as shown in the respective line in column 5 of Table I, application no. 20, has been published in B. And the activity of the gene product thereof is the activity of ATP-dependent RNA helicase.

[102121] Accordingly, in one embodiment, the process of the present invention for producing hentriacontane in a non-human organism, like a microorganism or a plant or a part thereof, comprises increasing or generating the activity of a gene product with the activity of a gene product conferring the activity of “ATP-dependent RNA helicase”, especially from Escherichia coli or its functional equivalent or its homolog, e.g. the increase of
[102122] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B1343, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B1343, and preferably the activity is increased non-targeted, or
[102123] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B1343, or a functional equivalent or a homolog thereof as depicted in column 8 of Table II, application no. 20, preferably a homolog or functional equivalent as depicted in column 8 of Table II B, application no. 20, and being depicted in the same respective line as said B1343, and preferably the activity is increased non-targeted, whereby the respective line disclose in column 7 the fine chemical hentriacontane.

[102124] Accordingly, in one embodiment, the molecule which activity is to be increased in the process of the invention is the gene product with an activity as a “ATP-dependent RNA helicase”, preferably it is the molecule of section (a) or (b) of this paragraph.

[102125] In particular, it was observed that in plants, especially in Arabidopsis thaliana, increasing or generating the activity of a gene product non-targeted with the activity of a “ATP-dependent RNA helicase”, preferably being encoded by a gene comprising the nucleic acid sequence SEQ ID NO. 76890, preferably the coding region thereof, conferred the production of or the increase in hentriacontane compared with the wild type control.

[102126] The nucleic acid sequence of B1445 from Escherichia coli, e.g. as shown in the respective line in column 5 of Table I, application no. 20, has been published in B. And the activity of the gene product thereof is the activity of b1445-protein.

[102127] Accordingly, in one embodiment, the process of the present invention for producing hentriacontane in a non-human organism, like a microorganism or a plant or a part thereof, comprises increasing or generating the activity of a gene product with the activity of a gene product conferring the activity of “b1445-protein”, especially from Escherichia coli or its functional equivalent or its homolog, e.g. the increase of
[102128] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B1445, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B1445, and preferably the activity is increased plastidic, or
[102129] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B1445, or a functional equivalent or a homolog thereof as depicted in column 8 of Table II, application no. 20, preferably a homolog or functional equivalent as depicted in column 8 of Table II B, application no. 20, and being depicted in the same respective line as said B1445, and preferably the activity is increased plastidic, whereby the respective line disclose in column 7 the fine chemical hentriacontane.

[102130] Accordingly, in one embodiment, the molecule which activity is to be increased in the process of the invention is the gene product with an activity as a “b1445-protein”, preferably it is the molecule of section (a) or (b) of this paragraph.

[102131] In particular, it was observed that in plants, especially in Arabidopsis thaliana, increasing or generating the activity of a gene product plastidic with the activity of a “b1445-protein”, preferably being encoded by a gene comprising the nucleic acid sequence SEQ ID NO. 38289, preferably the coding region thereof, conferred the production of or the increase in hentriacontane compared with the wild type control.

[102132] The nucleic acid sequence of B1597 from Escherichia coli, e.g. as shown in the respective line in column 5 of Table I, application no. 20, has been published in B. And the activity of the gene product thereof is the activity of acid shock protein.

[102133] Accordingly, in one embodiment, the process of the present invention for producing hentriacontane in a non-human organism, like a microorganism or a plant or a part thereof, comprises increasing or generating the activity of a gene product with the activity of a gene product conferring the activity of “acid shock protein”, especially from Escherichia coli or its functional equivalent or its homolog, e.g. the increase of
[102134] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B1597, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B1597, and preferably the activity is increased non-targeted, or
[102135] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B1597, or a functional equivalent or a homolog thereof as depicted in column 8 of Table II, application no. 20, preferably a homolog or functional equivalent as depicted in column 8 of Table II B, application no. 20, and being depicted in the same respective line as said B1597, and preferably the activity is increased non-targeted, whereby the respective line disclose in column 7 the fine chemical hentriacontane.

[102136] Accordingly, in one embodiment, the molecule which activity is to be increased in the process of the invention is the gene product with an activity as a “acid shock protein”, preferably it is the molecule of section (a) or (b) of this paragraph.

[102137] In particular, it was observed that in plants, especially in Arabidopsis thaliana, increasing or generating the activity of a gene product non-targeted with the activity of a “acid shock protein”, preferably being encoded by a gene comprising the nucleic acid sequence SEQ ID NO. 38300, preferably the coding region thereof, conferred the production of or the increase in hentriacontane compared with the wild type control.

[102138] The nucleic acid sequence of B1627 from Escherichia coli, e.g. as shown in the respective line in column 5 of Table I, application no. 20, has been published in B. And the activity of the gene product thereof is the activity of electron transport complex protein.

[102139] Accordingly, in one embodiment, the process of the present invention for producing hentriacontane in a non-human organism, like a microorganism or a plant or a part thereof, comprises increasing or generating the activity of a gene product with the activity of a gene product conferring the activity of “electron transport complex protein”, especially from Escherichia coli or its functional equivalent or its homolog, e.g. the increase of
[102140] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B1627, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B1627, and preferably the activity is increased plastidic, or
[102141] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B1627, or a functional equivalent or a homolog thereof as depicted in column 8 of Table II, application no. 20, preferably a homolog or functional equivalent as depicted in column 8 of Table II B, application no. 20, and being depicted in the same respective line as said B1627, and preferably the activity is increased plastidic, whereby the respective line disclose in column 7 the fine chemical hentriacontane.

[102142] Accordingly, in one embodiment, the molecule which activity is to be increased in the process of the invention is the gene product with an activity as a “electron transport complex protein”, preferably it is the molecule of section (a) or (b) of this paragraph.

[102143] In particular, it was observed that in plants, especially in Arabidopsis thaliana, increasing or generating the activity of a gene product plastidic with the activity of a “electron transport complex protein”, preferably being encoded by a gene comprising the nucleic acid sequence SEQ ID NO. 38345, preferably the coding region thereof, conferred the production of or the increase in hentriacontane compared with the wild type control.

[102144] The nucleic acid sequence of B1649 from Escherichia coli, e.g. as shown in the respective line in column 5 of Table I, application no. 20, has been published in B. And the activity of the gene product thereof is the activity of transcriptional regulator.

[102145] Accordingly, in one embodiment, the process of the present invention for producing hentriacontane in a non-human organism, like a microorganism or a plant or a part thereof, comprises increasing or generating the activity of a gene product with the activity of a gene product conferring the activity of “transcriptional regulator”, especially from Escherichia coli or its functional equivalent or its homolog, e.g. the increase of
[102146] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B1649, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B1649, and preferably the activity is increased plastidic, or
[102147] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B1649, or a functional equivalent or a homolog thereof as depicted in column 8 of Table II, application no. 20, preferably a homolog or functional equivalent as depicted in column 8 of Table II B, application no. 20, and being depicted in the same respective line as said B1649, and preferably the activity is increased plastidic, whereby the respective line disclose in column 7 the fine chemical hentriacontane.

[102148] Accordingly, in one embodiment, the molecule which activity is to be increased in the process of the invention is the gene product with an activity as a “transcriptional regulator”, preferably it is the molecule of section (a) or (b) of this paragraph.

[102149] In particular, it was observed that in plants, especially in Arabidopsis thaliana, increasing or generating the activity of a gene product plastidic with the activity of a “transcriptional regulator”, preferably being encoded by a gene comprising the nucleic acid sequence SEQ ID NO. 156392, preferably the coding region thereof, conferred the production of or the increase in hentriacontane compared with the wild type control.

[102150] The nucleic acid sequence of B1670 from Escherichia coli, e.g. as shown in the respective line in column 5 of Table I, application no. 20, has been published in B. And the activity of the gene product thereof is the activity of b1670-protein.

[102151] Accordingly, in one embodiment, the process of the present invention for producing hentriacontane in a non-human organism, like a microorganism or a plant or a part thereof, comprises increasing or generating the activity of a gene product with the activity of a gene product conferring the activity of “b1670-protein”, especially from Escherichia coli or its functional equivalent or its homolog, e.g. the increase of
[102152] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B1670, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B1670, and preferably the activity is increased non-targeted, or
[102153] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B1670, or a functional equivalent or a homolog thereof as depicted in column 8 of Table II, application no. 20, preferably a homolog or functional equivalent as depicted in column 8 of Table II B, application no. 20, and being depicted in the same respective line as said B1670, and preferably the activity is increased non-targeted, whereby the respective line disclose in column 7 the fine chemical hentriacontane.

[102154] Accordingly, in one embodiment, the molecule which activity is to be increased in the process of the invention is the gene product with an activity as a “b1670-protein”, preferably it is the molecule of section (a) or (b) of this paragraph.

[102155] In particular, it was observed that in plants, especially in Arabidopsis thaliana, increasing or generating the activity of a gene product non-targeted with the activity of a “b1670-protein”, preferably being encoded by a gene comprising the nucleic acid sequence SEQ ID NO. 78753, preferably the coding region thereof, conferred the production of or the increase in hentriacontane compared with the wild type control.

[102156] The nucleic acid sequence of B1704 from Escherichia coli, e.g. as shown in the respective line in column 5 of Table I, application no. 20, has been published in B. And the activity of the gene product thereof is the activity of phospho-2-dehydro-3-deoxyheptonate aldolase.

[102157] Accordingly, in one embodiment, the process of the present invention for producing hentriacontane in a non-human organism, like a microorganism or a plant or a part thereof, comprises increasing or generating the activity of a gene product with the activity of a gene product conferring the activity of “phospho-2-dehydro-3-deoxyheptonate aldolase”, especially from Escherichia coli or its functional equivalent or its homolog, e.g. the increase of
[102158] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B1704, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B1704, and preferably the activity is increased plastidic, or
[102159] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B1704, or a functional equivalent or a homolog thereof as depicted in column 8 of Table II, application no. 20, preferably a homolog or functional equivalent as depicted in column 8 of Table II B, application no. 20, and being depicted in the same respective line as said B1704, and preferably the activity is increased plastidic, whereby the respective line disclose in column 7 the fine chemical hentriacontane.

[102160] Accordingly, in one embodiment, the molecule which activity is to be increased in the process of the invention is the gene product with an activity as a “phospho-2-dehydro-3-deoxyheptonate aldolase”, preferably it is the molecule of section (a) or (b) of this paragraph.

[102161] In particular, it was observed that in plants, especially in Arabidopsis thaliana, increasing or generating the activity of a gene product plastidic with the activity of a “phospho-2-dehydro-3-deoxyheptonate aldolase”, preferably being encoded by a gene comprising the nucleic acid sequence SEQ ID NO. 156542, preferably the coding region thereof, conferred the production of or the increase in hentriacontane compared with the wild type control.

[102162] The nucleic acid sequence of B1930 from Escherichia coli, e.g. as shown in the respective line in column 5 of Table I, application no. 20, has been published in B. And the activity of the gene product thereof is the activity of b1930-protein.

[102163] Accordingly, in one embodiment, the process of the present invention for producing hentriacontane in a non-human organism, like a microorganism or a plant or a part thereof, comprises increasing or generating the activity of a gene product with the activity of a gene product conferring the activity of “b1930-protein”, especially from Escherichia coli or its functional equivalent or its homolog, e.g. the increase of
[102164] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B1930, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B1930, and preferably the activity is increased non-targeted, or
[102165] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B1930, or a functional equivalent or a homolog thereof as depicted in column 8 of Table II, application no. 20, preferably a homolog or functional equivalent as depicted in column 8 of Table II B, application no. 20, and being depicted in the same respective line as said B1930, and preferably the activity is increased non-targeted, whereby the respective line disclose in column 7 the fine chemical hentriacontane.

[102166] Accordingly, in one embodiment, the molecule which activity is to be increased in the process of the invention is the gene product with an activity as a “b1930-protein”, preferably it is the molecule of section (a) or (b) of this paragraph.

[102167] In particular, it was observed that in plants, especially in Arabidopsis thaliana, increasing or generating the activity of a gene product non-targeted with the activity of a “b1930-protein”, preferably being encoded by a gene comprising the nucleic acid sequence SEQ ID NO. 79181, preferably the coding region thereof, conferred the production of or the increase in hentriacontane compared with the wild type control.

[102168] The nucleic acid sequence of B2027 from Escherichia coli, e.g. as shown in the respective line in column 5 of Table I, application no. 20, has been published in B. And the activity of the gene product thereof is the activity of O-antigen chain length determinant.

[102169] Accordingly, in one embodiment, the process of the present invention for producing hentriacontane in a non-human organism, like a microorganism or a plant or a part thereof, comprises increasing or generating the activity of a gene product with the activity of a gene product conferring the activity of “O-antigen chain length determinant”, especially from Escherichia coli or its functional equivalent or its homolog, e.g. the increase of
[102170] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B2027, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B2027, and preferably the activity is increased non-targeted, or
[102171] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B2027, or a functional equivalent or a homolog thereof as depicted in column 8 of Table II, application no. 20, preferably a homolog or functional equivalent as depicted in column 8 of Table II B, application no. 20, and being depicted in the same respective line as said B2027, and preferably the activity is increased non-targeted, whereby the respective line disclose in column 7 the fine chemical hentriacontane.

[102172] Accordingly, in one embodiment, the molecule which activity is to be increased in the process of the invention is the gene product with an activity as a “O-antigen chain length determinant”, preferably it is the molecule of section (a) or (b) of this paragraph.

[102173] In particular, it was observed that in plants, especially in Arabidopsis thaliana, increasing or generating the activity of a gene product non-targeted with the activity of a “O-antigen chain length determinant”, preferably being encoded by a gene comprising the nucleic acid sequence SEQ ID NO. 79217, preferably the coding region thereof, conferred the production of or the increase in hentriacontane compared with the wild type control.

[102174] The nucleic acid sequence of B2053 from Escherichia coli, e.g. as shown in the respective line in column 5 of Table I, application no. 20, has been published in B. And the activity of the gene product thereof is the activity of GDP-mannose 4,6-dehydratase.

[102175] Accordingly, in one embodiment, the process of the present invention for producing hentriacontane in a non-human organism, like a microorganism or a plant or a part thereof, comprises increasing or generating the activity of a gene product with the activity of a gene product conferring the activity of “GDP-mannose 4,6-dehydratase”, especially from Escherichia coli or its functional equivalent or its homolog, e.g. the increase of
[102176] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B2053, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B2053, and preferably the activity is increased non-targeted, or
[102177] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B2053, or a functional equivalent or a homolog thereof as depicted in column 8 of Table II, application no. 20, preferably a homolog or functional equivalent as depicted in column 8 of Table II B, application no. 20, and being depicted in the same respective line as said B2053, and preferably the activity is increased non-targeted, whereby the respective line disclose in column 7 the fine chemical hentriacontane.

[102178] Accordingly, in one embodiment, the molecule which activity is to be increased in the process of the invention is the gene product with an activity as a “GDP-mannose 4,6-dehydratase”, preferably it is the molecule of section (a) or (b) of this paragraph.

[102179] In particular, it was observed that in plants, especially in Arabidopsis thaliana, increasing or generating the activity of a gene product non-targeted with the activity of a “GDP-mannose 4,6-dehydratase”, preferably being encoded by a gene comprising the nucleic acid sequence SEQ ID NO. 104277, preferably the coding region thereof, conferred the production of or the increase in hentriacontane compared with the wild type control.

[102180] The nucleic acid sequence of B2207 from Escherichia coli, e.g. as shown in the respective line in column 5 of Table I, application no. 20, has been published in B. And the activity of the gene product thereof is the activity of assembly protein for periplasmic nitrate reductase.

[102181] Accordingly, in one embodiment, the process of the present invention for producing hentriacontane in a non-human organism, like a microorganism or a plant or a part thereof, comprises increasing or generating the activity of a gene product with the activity of a gene product conferring the activity of “assembly protein for periplasmic nitrate reductase”, especially from Escherichia coli or its functional equivalent or its homolog, e.g. the increase of
[102182] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B2207, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B2207, and preferably the activity is increased plastidic, or
[102183] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B2207, or a functional equivalent or a homolog thereof as depicted in column 8 of Table II, application no. 20, preferably a homolog or functional equivalent as depicted in column 8 of Table II B, application no. 20, and being depicted in the same respective line as said B2207, and preferably the activity is increased plastidic, whereby the respective line disclose in column 7 the fine chemical hentriacontane.

[102184] Accordingly, in one embodiment, the molecule which activity is to be increased in the process of the invention is the gene product with an activity as a “assembly protein for periplasmic nitrate reductase”, preferably it is the molecule of section (a) or (b) of this paragraph.

[102185] In particular, it was observed that in plants, especially in Arabidopsis thaliana, increasing or generating the activity of a gene product plastidic with the activity of a “assembly protein for periplasmic nitrate reductase”, preferably being encoded by a gene comprising the nucleic acid sequence SEQ ID NO. 156810, preferably the coding region thereof, conferred the production of or the increase in hentriacontane compared with the wild type control.

[102186] The nucleic acid sequence of B2389 from Escherichia coli, e.g. as shown in the respective line in column 5 of Table I, application no. 20, has been published in B. And the activity of the gene product thereof is the activity of ion-transport protein.

[102187] Accordingly, in one embodiment, the process of the present invention for producing hentriacontane in a non-human organism, like a microorganism or a plant or a part thereof, comprises increasing or generating the activity of a gene product with the activity of a gene product conferring the activity of “ion-transport protein”, especially from Escherichia coli or its functional equivalent or its homolog, e.g. the increase of
[102188] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B2389, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B2389, and preferably the activity is increased non-targeted, or
[102189] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B2389, or a functional equivalent or a homolog thereof as depicted in column 8 of Table II, application no. 20, preferably a homolog or functional equivalent as depicted in column 8 of Table II B, application no. 20, and being depicted in the same respective line as said B2389, and preferably the activity is increased non-targeted, whereby the respective line disclose in column 7 the fine chemical hentriacontane.

[102190] Accordingly, in one embodiment, the molecule which activity is to be increased in the process of the invention is the gene product with an activity as a “ion-transport protein”, preferably it is the molecule of section (a) or (b) of this paragraph.

[102191] In particular, it was observed that in plants, especially in Arabidopsis thaliana, increasing or generating the activity of a gene product non-targeted with the activity of a “ion-transport protein”, preferably being encoded by a gene comprising the nucleic acid sequence SEQ ID NO. 146238, preferably the coding region thereof, conferred the production of or the increase in hentriacontane compared with the wild type control.

[102192] The nucleic acid sequence of B2399 from Escherichia coli, e.g. as shown in the respective line in column 5 of Table I, application no. 20, has been published in B. And the activity of the gene product thereof is the activity of b2399-protein.

[102193] Accordingly, in one embodiment, the process of the present invention for producing hentriacontane in a non-human organism, like a microorganism or a plant or a part thereof, comprises increasing or generating the activity of a gene product with the activity of a gene product conferring the activity of “b2399-protein”, especially from Escherichia coli or its functional equivalent or its homolog, e.g. the increase of
[102194] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B2399, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B2399, and preferably the activity is increased non-targeted, or
[102195] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B2399, or a functional equivalent or a homolog thereof as depicted in column 8 of Table II, application no. 20, preferably a homolog or functional equivalent as depicted in column 8 of Table II B, application no. 20, and being depicted in the same respective line as said B2399, and preferably the activity is increased non-targeted, whereby the respective line disclose in column 7 the fine chemical hentriacontane.

[102196] Accordingly, in one embodiment, the molecule which activity is to be increased in the process of the invention is the gene product with an activity as a “b2399-protein”, preferably it is the molecule of section (a) or (b) of this paragraph.

[102197] In particular, it was observed that in plants, especially in Arabidopsis thaliana, increasing or generating the activity of a gene product non-targeted with the activity of a “b2399-protein”, preferably being encoded by a gene comprising the nucleic acid sequence SEQ ID NO. 39237, preferably the coding region thereof, conferred the production of or the increase in hentriacontane compared with the wild type control.

[102198] The nucleic acid sequence of B2414 from Escherichia coli, e.g. as shown in the respective line in column 5 of Table I, application no. 20, has been published in B. And the activity of the gene product thereof is the activity of cysteine synthase A.

[102199] Accordingly, in one embodiment, the process of the present invention for producing hentriacontane in a non-human organism, like a microorganism or a plant or a part thereof, comprises increasing or generating the activity of a gene product with the activity of a gene product conferring the activity of “cysteine synthase A”, especially from Escherichia coli or its functional equivalent or its homolog, e.g. the increase of
[102200] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B2414, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B2414, and preferably the activity is increased non-targeted, or
[102201] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B2414, or a functional equivalent or a homolog thereof as depicted in column 8 of Table II, application no. 20, preferably a homolog or functional equivalent as depicted in column 8 of Table II B, application no. 20, and being depicted in the same respective line as said B2414, and preferably the activity is increased non-targeted, whereby the respective line disclose in column 7 the fine chemical hentriacontane.

[102202] Accordingly, in one embodiment, the molecule which activity is to be increased in the process of the invention is the gene product with an activity as a “cysteine synthase A”, preferably it is the molecule of section (a) or (b) of this paragraph.

[102203] In particular, it was observed that in plants, especially in Arabidopsis thaliana, increasing or generating the activity of a gene product non-targeted with the activity of a “cysteine synthase A”, preferably being encoded by a gene comprising the nucleic acid sequence SEQ ID NO. 39300, preferably the coding region thereof, conferred the production of or the increase in hentriacontane compared with the wild type control.

[102204] The nucleic acid sequence of B2439 from Escherichia coli, e.g. as shown in the respective line in column 5 of Table I, application no. 20, has been published in B. And the activity of the gene product thereof is the activity of ethanolamine utilization protein.

[102205] Accordingly, in one embodiment, the process of the present invention for producing hentriacontane in a non-human organism, like a microorganism or a plant or a part thereof, comprises increasing or generating the activity of a gene product with the activity of a gene product conferring the activity of “ethanolamine utilization protein”, especially from Escherichia coli or its functional equivalent or its homolog, e.g. the increase of
[102206] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B2439, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B2439, and preferably the activity is increased non-targeted, or
[102207] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B2439, or a functional equivalent or a homolog thereof as depicted in column 8 of Table II, application no. 20, preferably a homolog or functional equivalent as depicted in column 8 of Table II B, application no. 20, and being depicted in the same respective line as said B2439, and preferably the activity is increased non-targeted, whereby the respective line disclose in column 7 the fine chemical hentriacontane.

[102208] Accordingly, in one embodiment, the molecule which activity is to be increased in the process of the invention is the gene product with an activity as a “ethanolamine utilization protein”, preferably it is the molecule of section (a) or (b) of this paragraph.

[102209] In particular, it was observed that in plants, especially in Arabidopsis thaliana, increasing or generating the activity of a gene product non-targeted with the activity of a “ethanolamine utilization protein”, preferably being encoded by a gene comprising the nucleic acid sequence SEQ ID NO. 115628, preferably the coding region thereof, conferred the production of or the increase in hentriacontane compared with the wild type control.

[102210] The nucleic acid sequence of B2554 from Escherichia coli, e.g. as shown in the respective line in column 5 of Table I, application no. 20, has been published in B. And the activity of the gene product thereof is the activity of sigma-54 dependent response regulator.

[102211] Accordingly, in one embodiment, the process of the present invention for producing hentriacontane in a non-human organism, like a microorganism or a plant or a part thereof, comprises increasing or generating the activity of a gene product with the activity of a gene product conferring the activity of “sigma-54 dependent response regulator”, especially from Escherichia coli or its functional equivalent or its homolog, e.g. the increase of
[102212] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B2554, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B2554, and preferably the activity is increased plastidic, or
[102213] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B2554, or a functional equivalent or a homolog thereof as depicted in column 8 of Table II, application no. 20, preferably a homolog or functional equivalent as depicted in column 8 of Table II B, application no. 20, and being depicted in the same respective line as said B2554, and preferably the activity is increased plastidic, whereby the respective line disclose in column 7 the fine chemical hentriacontane.

[102214] Accordingly, in one embodiment, the molecule which activity is to be increased in the process of the invention is the gene product with an activity as a “sigma-54 dependent response regulator”, preferably it is the molecule of section (a) or (b) of this paragraph.

[102215] In particular, it was observed that in plants, especially in Arabidopsis thaliana, increasing or generating the activity of a gene product plastidic with the activity of a “sigma-54 dependent response regulator”, preferably being encoded by a gene comprising the nucleic acid sequence SEQ ID NO. 156910, preferably the coding region thereof, conferred the production of or the increase in hentriacontane compared with the wild type control.

[102216] The nucleic acid sequence of B2653 from Escherichia coli, e.g. as shown in the respective line in column 5 of Table I, application no. 20, has been published in B. And the activity of the gene product thereof is the activity of b2653-protein.

[102217] Accordingly, in one embodiment, the process of the present invention for producing hentriacontane in a non-human organism, like a microorganism or a plant or a part thereof, comprises increasing or generating the activity of a gene product with the activity of a gene product conferring the activity of “b2653-protein”, especially from Escherichia coli or its functional equivalent or its homolog, e.g. the increase of
[102218] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B2653, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B2653, and preferably the activity is increased non-targeted, or
[102219] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B2653, or a functional equivalent or a homolog thereof as depicted in column 8 of Table II, application no. 20, preferably a homolog or functional equivalent as depicted in column 8 of Table II B, application no. 20, and being depicted in the same respective line as said B2653, and preferably the activity is increased non-targeted, whereby the respective line disclose in column 7 the fine chemical hentriacontane.

[102220] Accordingly, in one embodiment, the molecule which activity is to be increased in the process of the invention is the gene product with an activity as a “b2653-protein”, preferably it is the molecule of section (a) or (b) of this paragraph.

[102221] In particular, it was observed that in plants, especially in Arabidopsis thaliana, increasing or generating the activity of a gene product non-targeted with the activity of a “b2653-protein”, preferably being encoded by a gene comprising the nucleic acid sequence SEQ ID NO. 146288, preferably the coding region thereof, conferred the production of or the increase in hentriacontane compared with the wild type control.

[102222] The nucleic acid sequence of B2762 from Escherichia coli, e.g. as shown in the respective line in column 5 of Table I, application no. 20, has been published in B. And the activity of the gene product thereof is the activity of phosphoadenosine phosphosulfate reductase.

[102223] Accordingly, in one embodiment, the process of the present invention for producing hentriacontane in a non-human organism, like a microorganism or a plant or a part thereof, comprises increasing or generating the activity of a gene product with the activity of a gene product conferring the activity of “phosphoadenosine phosphosulfate reductase”, especially from Escherichia coli or its functional equivalent or its homolog, e.g. the increase of
[102224] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B2762, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B2762, and preferably the activity is increased non-targeted, or
[102225] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B2762, or a functional equivalent or a homolog thereof as depicted in column 8 of Table II, application no. 20, preferably a homolog or functional equivalent as depicted in column 8 of Table II B, application no. 20, and being depicted in the same respective line as said B2762, and preferably the activity is increased non-targeted, whereby the respective line disclose in column 7 the fine chemical hentriacontane.

[102226] Accordingly, in one embodiment, the molecule which activity is to be increased in the process of the invention is the gene product with an activity as a “phosphoadenosine phosphosulfate reductase”, preferably it is the molecule of section (a) or (b) of this paragraph.

[102227] In particular, it was observed that in plants, especially in Arabidopsis thaliana, increasing or generating the activity of a gene product non-targeted with the activity of a “phosphoadenosine phosphosulfate reductase”, preferably being encoded by a gene comprising the nucleic acid sequence SEQ ID NO. 81451, preferably the coding region thereof, conferred the production of or the increase in hentriacontane compared with the wild type control.

[102228] The nucleic acid sequence of B2818 from Escherichia coli, e.g. as shown in the respective line in column 5 of Table I, application no. 20, has been published in B. And the activity of the gene product thereof is the activity of amino-acid acetyltransferase.

[102229] Accordingly, in one embodiment, the process of the present invention for producing hentriacontane in a non-human organism, like a microorganism or a plant or a part thereof, comprises increasing or generating the activity of a gene product with the activity of a gene product conferring the activity of “amino-acid acetyltransferase”, especially from Escherichia coli or its functional equivalent or its homolog, e.g. the increase of
[102230] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B2818, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B2818, and preferably the activity is increased non-targeted, or
[102231] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B2818, or a functional equivalent or a homolog thereof as depicted in column 8 of Table II, application no. 20, preferably a homolog or functional equivalent as depicted in column 8 of Table II B, application no. 20, and being depicted in the same respective line as said B2818, and preferably the activity is increased non-targeted, whereby the respective line disclose in column 7 the fine chemical hentriacontane.

[102232] Accordingly, in one embodiment, the molecule which activity is to be increased in the process of the invention is the gene product with an activity as a “amino-acid acetyltransferase”, preferably it is the molecule of section (a) or (b) of this paragraph.

[102233] In particular, it was observed that in plants, especially in Arabidopsis thaliana, increasing or generating the activity of a gene product non-targeted with the activity of a “amino-acid acetyltransferase”, preferably being encoded by a gene comprising the nucleic acid sequence SEQ ID NO. 81576, preferably the coding region thereof, conferred the production of or the increase in hentriacontane compared with the wild type control.

[102234] The nucleic acid sequence of B2822 from Escherichia coli, e.g. as shown in the respective line in column 5 of Table I, application no. 20, has been published in B. And the activity of the gene product thereof is the activity of exonuclease subunit.

[102235] Accordingly, in one embodiment, the process of the present invention for producing hentriacontane in a non-human organism, like a microorganism or a plant or a part thereof, comprises increasing or generating the activity of a gene product with the activity of a gene product conferring the activity of “exonuclease subunit”, especially from Escherichia coli or its functional equivalent or its homolog, e.g. the increase of
[102236] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B2822, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B2822, and preferably the activity is increased non-targeted, or
[102237] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B2822, or a functional equivalent or a homolog thereof as depicted in column 8 of Table II, application no. 20, preferably a homolog or functional equivalent as depicted in column 8 of Table II B, application no. 20, and being depicted in the same respective line as said B2822, and preferably the activity is increased non-targeted, whereby the respective line disclose in column 7 the fine chemical hentriacontane.

[102238] Accordingly, in one embodiment, the molecule which activity is to be increased in the process of the invention is the gene product with an activity as a “exonuclease subunit”, preferably it is the molecule of section (a) or (b) of this paragraph.

[102239] In particular, it was observed that in plants, especially in Arabidopsis thaliana, increasing or generating the activity of a gene product non-targeted with the activity of a “exonuclease subunit”, preferably being encoded by a gene comprising the nucleic acid sequence SEQ ID NO. 157128, preferably the coding region thereof, conferred the production of or the increase in hentriacontane compared with the wild type control.

[102240] The nucleic acid sequence of B2957 from Escherichia coli, e.g. as shown in the respective line in column 5 of Table I, application no. 20, has been published in B. And the activity of the gene product thereof is the activity of L-asparaginase.

[102241] Accordingly, in one embodiment, the process of the present invention for producing hentriacontane in a non-human organism, like a microorganism or a plant or a part thereof, comprises increasing or generating the activity of a gene product with the activity of a gene product conferring the activity of “L-asparaginase”, especially from Escherichia coli or its functional equivalent or its homolog, e.g. the increase of
[102242] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B2957, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B2957, and preferably the activity is increased non-targeted, or
[102243] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B2957, or a functional equivalent or a homolog thereof as depicted in column 8 of Table II, application no. 20, preferably a homolog or functional equivalent as depicted in column 8 of Table II B, application no. 20, and being depicted in the same respective line as said B2957, and preferably the activity is increased non-targeted, whereby the respective line disclose in column 7 the fine chemical hentriacontane.

[102244] Accordingly, in one embodiment, the molecule which activity is to be increased in the process of the invention is the gene product with an activity as a “L-asparaginase”, preferably it is the molecule of section (a) or (b) of this paragraph.

[102245] In particular, it was observed that in plants, especially in Arabidopsis thaliana, increasing or generating the activity of a gene product non-targeted with the activity of a “L-asparaginase”, preferably being encoded by a gene comprising the nucleic acid sequence SEQ ID NO. 41499, preferably the coding region thereof, conferred the production of or the increase in hentriacontane compared with the wild type control.

[102246] The nucleic acid sequence of B2965 from Escherichia coli, e.g. as shown in the respective line in column 5 of Table I, application no. 20, has been published in B. And the activity of the gene product thereof is the activity of ornithine decarboxylase.

[102247] Accordingly, in one embodiment, the process of the present invention for producing hentriacontane in a non-human organism, like a microorganism or a plant or a part thereof, comprises increasing or generating the activity of a gene product with the activity of a gene product conferring the activity of “ornithine decarboxylase”, especially from Escherichia coli or its functional equivalent or its homolog, e.g. the increase of
[102248] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B2965, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B2965, and preferably the activity is increased plastidic, or
[102249] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B2965, or a functional equivalent or a homolog thereof as depicted in column 8 of Table II, application no. 20, preferably a homolog or functional equivalent as depicted in column 8 of Table II B, application no. 20, and being depicted in the same respective line as said B2965, and preferably the activity is increased plastidic, whereby the respective line disclose in column 7 the fine chemical hentriacontane.

[102250] Accordingly, in one embodiment, the molecule which activity is to be increased in the process of the invention is the gene product with an activity as a “ornithine decarboxylase”, preferably it is the molecule of section (a) or (b) of this paragraph.

[102251] In particular, it was observed that in plants, especially in Arabidopsis thaliana, increasing or generating the activity of a gene product plastidic with the activity of a “ornithine decarboxylase”, preferably being encoded by a gene comprising the nucleic acid sequence SEQ ID NO. 157321, preferably the coding region thereof, conferred the production of or the increase in hentriacontane compared with the wild type control.

[102252] The nucleic acid sequence of B3385 from Escherichia coli, e.g. as shown in the respective line in column 5 of Table I, application no. 20, has been published in B. And the activity of the gene product thereof is the activity of phosphoglycolate phosphatase.

[102253] Accordingly, in one embodiment, the process of the present invention for producing hentriacontane in a non-human organism, like a microorganism or a plant or a part thereof, comprises increasing or generating the activity of a gene product with the activity of a gene product conferring the activity of “phosphoglycolate phosphatase”, especially from Escherichia coli or its functional equivalent or its homolog, e.g. the increase of
[102254] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B3385, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B3385, and preferably the activity is increased plastidic, or
[102255] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B3385, or a functional equivalent or a homolog thereof as depicted in column 8 of Table II, application no. 20, preferably a homolog or functional equivalent as depicted in column 8 of Table II B, application no. 20, and being depicted in the same respective line as said B3385, and preferably the activity is increased plastidic, whereby the respective line disclose in column 7 the fine chemical hentriacontane.

[102256] Accordingly, in one embodiment, the molecule which activity is to be increased in the process of the invention is the gene product with an activity as a “phosphoglycolate phosphatase”, preferably it is the molecule of section (a) or (b) of this paragraph.

[102257] In particular, it was observed that in plants, especially in Arabidopsis thaliana, increasing or generating the activity of a gene product plastidic with the activity of a “phosphoglycolate phosphatase”, preferably being encoded by a gene comprising the nucleic acid sequence SEQ ID NO. 157450, preferably the coding region thereof, conferred the production of or the increase in hentriacontane compared with the wild type control.

[102258] The nucleic acid sequence of B3568 from Escherichia coli, e.g. as shown in the respective line in column 5 of Table I, application no. 20, has been published in B. And the activity of the gene product thereof is the activity of sugar transport system permease protein.

[102259] Accordingly, in one embodiment, the process of the present invention for producing hentriacontane in a non-human organism, like a microorganism or a plant or a part thereof, comprises increasing or generating the activity of a gene product with the activity of a gene product conferring the activity of “sugar transport system permease protein”, especially from Escherichia coli or its functional equivalent or its homolog, e.g. the increase of
[102260] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B3568, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B3568, and preferably the activity is increased plastidic, or
[102261] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B3568, or a functional equivalent or a homolog thereof as depicted in column 8 of Table II, application no. 20, preferably a homolog or functional equivalent as depicted in column 8 of Table II B, application no. 20, and being depicted in the same respective line as said B3568, and preferably the activity is increased plastidic, whereby the respective line disclose in column 7 the fine chemical hentriacontane.

[102262] Accordingly, in one embodiment, the molecule which activity is to be increased in the process of the invention is the gene product with an activity as a “sugar transport system permease protein”, preferably it is the molecule of section (a) or (b) of this paragraph.

[102263] In particular, it was observed that in plants, especially in Arabidopsis thaliana, increasing or generating the activity of a gene product plastidic with the activity of a “sugar transport system permease protein”, preferably being encoded by a gene comprising the nucleic acid sequence SEQ ID NO. 125264, preferably the coding region thereof, conferred the production of or the increase in hentriacontane compared with the wild type control.

[102264] The nucleic acid sequence of GM02LC44512 from Glycine max, e.g. as shown in the respective line in column 5 of Table I, application no. 20, is unpublished. And the activity of the gene product thereof is the activity of cyclin D.

[102265] Accordingly, in one embodiment, the process of the present invention for producing hentriacontane in a non-human organism, like a microorganism or a plant or a part thereof, comprises increasing or generating the activity of a gene product with the activity of a gene product conferring the activity of “cyclin D”, especially from Glycine max or its functional equivalent or its homolog, e.g. the increase of
[102266] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said GM02LC44512, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said GM02LC44512, and preferably the activity is increased non-targeted, or
[102267] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said GM02LC44512, or a functional equivalent or a homolog thereof as depicted in column 8 of Table II, application no. 20, preferably a homolog or functional equivalent as depicted in column 8 of Table II B, application no. 20, and being depicted in the same respective line as said GM02LC44512, and preferably the activity is increased non-targeted, whereby the respective line disclose in column 7 the fine chemical hentriacontane.

[102268] Accordingly, in one embodiment, the molecule which activity is to be increased in the process of the invention is the gene product with an activity as a “cyclin D”, preferably it is the molecule of section (a) or (b) of this paragraph.

[102269] In particular, it was observed that in plants, especially in Arabidopsis thaliana, increasing or generating the activity of a gene product non-targeted with the activity of a “cyclin D”, preferably being encoded by a gene comprising the nucleic acid sequence SEQ ID NO. 46850, preferably the coding region thereof, conferred the production of or the increase in hentriacontane compared with the wild type control.

[102270] The nucleic acid sequence of Slr2124 from Synechocystis sp., e.g. as shown in the respective line in column 5 of Table I, application no. 20, has been published in C. And the activity of the gene product thereof is the activity of short-chain alcohol dehydrogenase family.

[102271] Accordingly, in one embodiment, the process of the present invention for producing hentriacontane in a non-human organism, like a microorganism or a plant or a part thereof, comprises increasing or generating the activity of a gene product with the activity of a gene product conferring the activity of “short-chain alcohol dehydrogenase family”, especially from Synechocystis sp. or its functional equivalent or its homolog, e.g. the increase of
[102272] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said Slr2124, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said Slr2124, and preferably the activity is increased plastidic, or
[102273] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said Slr2124, or a functional equivalent or a homolog thereof as depicted in column 8 of Table II, application no. 20, preferably a homolog or functional equivalent as depicted in column 8 of Table II B, application no. 20, and being depicted in the same respective line as said Slr2124, and preferably the activity is increased plastidic, whereby the respective line disclose in column 7 the fine chemical hentriacontane.

[102274] Accordingly, in one embodiment, the molecule which activity is to be increased in the process of the invention is the gene product with an activity as a “short-chain alcohol dehydrogenase family”, preferably it is the molecule of section (a) or (b) of this paragraph.

[102275] In particular, it was observed that in plants, especially in Arabidopsis thaliana, increasing or generating the activity of a gene product plastidic with the activity of a “short-chain alcohol dehydrogenase family”, preferably being encoded by a gene comprising the nucleic acid sequence SEQ ID NO. 59370, preferably the coding region thereof, conferred the production of or the increase in hentriacontane compared with the wild type control.

[102276] The nucleic acid sequence of TTC0035 from Thermus thermophilus, e.g. as shown in the respective line in column 5 of Table I, application no. 20, has been published in E. And the activity of the gene product thereof is the activity of cell division protein.

[102277] Accordingly, in one embodiment, the process of the present invention for producing hentriacontane in a non-human organism, like a microorganism or a plant or a part thereof, comprises increasing or generating the activity of a gene product with the activity of a gene product conferring the activity of “cell division protein”, especially from Thermus thermophilus or its functional equivalent or its homolog, e.g. the increase of
[102278] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said TTC0035, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said TTC0035, and preferably the activity is increased non-targeted, or
[102279] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said TTC0035, or a functional equivalent or a homolog thereof as depicted in column 8 of Table II, application no. 20, preferably a homolog or functional equivalent as depicted in column 8 of Table II B, application no. 20, and being depicted in the same respective line as said TTC0035, and preferably the activity is increased non-targeted, whereby the respective line disclose in column 7 the fine chemical hentriacontane.

[102280] Accordingly, in one embodiment, the molecule which activity is to be increased in the process of the invention is the gene product with an activity as a “cell division protein”, preferably it is the molecule of section (a) or (b) of this paragraph.

[102281] In particular, it was observed that in plants, especially in Arabidopsis thaliana, increasing or generating the activity of a gene product non-targeted with the activity of a “cell division protein”, preferably being encoded by a gene comprising the nucleic acid sequence SEQ ID NO. 60301, preferably the coding region thereof, conferred the production of or the increase in hentriacontane compared with the wild type control.

[102282] The nucleic acid sequence of TTC0917 from Thermus thermophilus, e.g. as shown in the respective line in column 5 of Table I, application no. 20, has been published in E. And the activity of the gene product thereof is the activity of metal-dependent hydrolase.

[102283] Accordingly, in one embodiment, the process of the present invention for producing hentriacontane in a non-human organism, like a microorganism or a plant or a part thereof, comprises increasing or generating the activity of a gene product with the activity of a gene product conferring the activity of “metal-dependent hydrolase”, especially from Thermus thermophilus or its functional equivalent or its homolog, e.g. the increase of
[102284] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said TTC0917, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said TTC0917, and preferably the activity is increased non-targeted, or
[102285] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said TTC0917, or a functional equivalent or a homolog thereof as depicted in column 8 of Table II, application no. 20, preferably a homolog or functional equivalent as depicted in column 8 of Table II B, application no. 20, and being depicted in the same respective line as said TTC0917, and preferably the activity is increased non-targeted, whereby the respective line disclose in column 7 the fine chemical hentriacontane.

[102286] Accordingly, in one embodiment, the molecule which activity is to be increased in the process of the invention is the gene product with an activity as a “metal-dependent hydrolase”, preferably it is the molecule of section (a) or (b) of this paragraph.

[102287] In particular, it was observed that in plants, especially in Arabidopsis thaliana, increasing or generating the activity of a gene product non-targeted with the activity of a “metal-dependent hydrolase”, preferably being encoded by a gene comprising the nucleic acid sequence SEQ ID NO. 61553, preferably the coding region thereof, conferred the production of or the increase in hentriacontane compared with the wild type control.

[102288] The nucleic acid sequence of Ybr249c from Saccharomyces cerevisiae, e.g. as shown in the respective line in column 5 of Table I, application no. 20, has been published in A. And the activity of the gene product thereof is the activity of 3-deoxy-7-phosphoheptulonate synthase.

[102289] Accordingly, in one embodiment, the process of the present invention for producing hentriacontane in a non-human organism, like a microorganism or a plant or a part thereof, comprises increasing or generating the activity of a gene product with the activity of a gene product conferring the activity of “3-deoxy-7-phosphoheptulonate synthase”, especially from Saccharomyces cerevisiae or its functional equivalent or its homolog, e.g. the increase of
[102290] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said Ybr249c, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said Ybr249c, and preferably the activity is increased plastidic, or
[102291] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said Ybr249c, or a functional equivalent or a homolog thereof as depicted in column 8 of Table II, application no. 20, preferably a homolog or functional equivalent as depicted in column 8 of Table II B, application no. 20, and being depicted in the same respective line as said Ybr249c, and preferably the activity is increased plastidic, whereby the respective line disclose in column 7 the fine chemical hentriacontane.

[102292] Accordingly, in one embodiment, the molecule which activity is to be increased in the process of the invention is the gene product with an activity as a “3-deoxy-7-phosphoheptulonate synthase”, preferably it is the molecule of section (a) or (b) of this paragraph.

[102293] In particular, it was observed that in plants, especially in Arabidopsis thaliana, increasing or generating the activity of a gene product plastidic with the activity of a “3-deoxy-7-phosphoheptulonate synthase”, preferably being encoded by a gene comprising the nucleic acid sequence SEQ ID NO. 88879, preferably the coding region thereof, conferred the production of or the increase in hentriacontane compared with the wild type control.

[102294] The nucleic acid sequence of Yfl019c from Saccharomyces cerevisiae, e.g. as shown in the respective line in column 5 of Table I, application no. 20, has been published in A. And the activity of the gene product thereof is the activity of YFL019C-protein.

[102295] Accordingly, in one embodiment, the process of the present invention for producing hentriacontane in a non-human organism, like a microorganism or a plant or a part thereof, comprises increasing or generating the activity of a gene product with the activity of a gene product conferring the activity of “YFL019C-protein”, especially from Saccharomyces cerevisiae or its functional equivalent or its homolog, e.g. the increase of
[102296] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said Yfl019c, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said Yfl019c, and preferably the activity is increased non-targeted, or
[102297] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said Yfl019c, or a functional equivalent or a homolog thereof as depicted in column 8 of Table II, application no. 20, preferably a homolog or functional equivalent as depicted in column 8 of Table II B, application no. 20, and being depicted in the same respective line as said Yfl019c, and preferably the activity is increased non-targeted, whereby the respective line disclose in column 7 the fine chemical hentriacontane.

[102298] Accordingly, in one embodiment, the molecule which activity is to be increased in the process of the invention is the gene product with an activity as a “YFL0190-protein”, preferably it is the molecule of section (a) or (b) of this paragraph.

[102299] In particular, it was observed that in plants, especially in Arabidopsis thaliana, increasing or generating the activity of a gene product non-targeted with the activity of a “YFL019C-protein”, preferably being encoded by a gene comprising the nucleic acid sequence SEQ ID NO. 64144, preferably the coding region thereof, conferred the production of or the increase in hentriacontane compared with the wild type control.

[102300] The nucleic acid sequence of Yfl053w from Saccharomyces cerevisiae, e.g. as shown in the respective line in column 5 of Table I, application no. 20, has been published in A. And the activity of the gene product thereof is the activity of dihydroxyacetone kinase.

[102301] Accordingly, in one embodiment, the process of the present invention for producing hentriacontane in a non-human organism, like a microorganism or a plant or a part thereof, comprises increasing or generating the activity of a gene product with the activity of a gene product conferring the activity of “dihydroxyacetone kinase”, especially from Saccharomyces cerevisiae or its functional equivalent or its homolog, e.g. the increase of
[102302] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said Yfl053w, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said Yfl053w, and preferably the activity is increased non-targeted, or
[102303] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said Yfl053w, or a functional equivalent or a homolog thereof as depicted in column 8 of Table II, application no. 20, preferably a homolog or functional equivalent as depicted in column 8 of Table II B, application no. 20, and being depicted in the same respective line as said Yfl053w, and preferably the activity is increased non-targeted, whereby the respective line disclose in column 7 the fine chemical hentriacontane.

[102304] Accordingly, in one embodiment, the molecule which activity is to be increased in the process of the invention is the gene product with an activity as a “dihydroxyacetone kinase”, preferably it is the molecule of section (a) or (b) of this paragraph.

[102305] In particular, it was observed that in plants, especially in Arabidopsis thaliana, increasing or generating the activity of a gene product non-targeted with the activity of a “dihydroxyacetone kinase”, preferably being encoded by a gene comprising the nucleic acid sequence SEQ ID NO. 120309, preferably the coding region thereof, conferred the production of or the increase in hentriacontane compared with the wild type control.

[102306] The nucleic acid sequence of YGRO55W from Saccharomyces cerevisiae, e.g. as shown in the respective line in column 5 of Table I, application no. 20, has been published in A. And the activity of the gene product thereof is the activity of methionine permease.

[102307] Accordingly, in one embodiment, the process of the present invention for producing hentriacontane in a non-human organism, like a microorganism or a plant or a part thereof, comprises increasing or generating the activity of a gene product with the activity of a gene product conferring the activity of “methionine permease”, especially from Saccharomyces cerevisiae or its functional equivalent or its homolog, e.g. the increase of
[102308] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said YGRO55W, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said YGRO55W, and preferably the activity is increased plastidic, or
[102309] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said YGRO55W, or a functional equivalent or a homolog thereof as depicted in column 8 of Table II, application no. 20, preferably a homolog or functional equivalent as depicted in column 8 of Table II B, application no. 20, and being depicted in the same respective line as said YGRO55W, and preferably the activity is increased plastidic, whereby the respective line disclose in column 7 the fine chemical hentriacontane.

[102310] Accordingly, in one embodiment, the molecule which activity is to be increased in the process of the invention is the gene product with an activity as a “methionine permease”, preferably it is the molecule of section (a) or (b) of this paragraph.

[102311] In particular, it was observed that in plants, especially in Arabidopsis thaliana, increasing or generating the activity of a gene product plastidic with the activity of a “methionine permease”, preferably being encoded by a gene comprising the nucleic acid sequence SEQ ID NO. 157717, preferably the coding region thereof, conferred the production of or the increase in hentriacontane compared with the wild type control.

[102312] The nucleic acid sequence of YHL039W from Saccharomyces cerevisiae, e.g. as shown in the respective line in column 5 of Table I, application no. 20, has been published in A. And the activity of the gene product thereof is the activity of YHL039W-protein.

[102313] Accordingly, in one embodiment, the process of the present invention for producing hentriacontane in a non-human organism, like a microorganism or a plant or a part thereof, comprises increasing or generating the activity of a gene product with the activity of a gene product conferring the activity of “YHL039W-protein”, especially from Saccharomyces cerevisiae or its functional equivalent or its homolog, e.g. the increase of
[102314] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said YHL039W, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said YHL039W, and preferably the activity is increased plastidic, or
[102315] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said YHL039W, or a functional equivalent or a homolog thereof as depicted in column 8 of Table II, application no. 20, preferably a homolog or functional equivalent as depicted in column 8 of Table II B, application no. 20, and being depicted in the same respective line as said YHL039W, and preferably the activity is increased plastidic, whereby the respective line disclose in column 7 the fine chemical hentriacontane.

[102316] Accordingly, in one embodiment, the molecule which activity is to be increased in the process of the invention is the gene product with an activity as a “YHL039W-protein”, preferably it is the molecule of section (a) or (b) of this paragraph.

[102317] In particular, it was observed that in plants, especially in Arabidopsis thaliana, increasing or generating the activity of a gene product plastidic with the activity of a “YHL039W-protein”, preferably being encoded by a gene comprising the nucleic acid sequence SEQ ID NO. 157832, preferably the coding region thereof, conferred the production of or the increase in hentriacontane compared with the wild type control.

[102318] The nucleic acid sequence of Yhr022c-a from Saccharomyces cerevisiae, e.g. as shown in the respective line in column 5 of Table I, application no. 20, has been published in A. And the activity of the gene product thereof is the activity of YHR022C-A-protein.

[102319] Accordingly, in one embodiment, the process of the present invention for producing hentriacontane in a non-human organism, like a microorganism or a plant or a part thereof, comprises increasing or generating the activity of a gene product with the activity of a gene product conferring the activity of “YHR022C-A-protein”, especially from Saccharomyces cerevisiae or its functional equivalent or its homolog, e.g. the increase of
[102320] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said Yhr022c-a, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said Yhr022c-a, and preferably the activity is increased non-targeted, or
[102321] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said Yhr022c-a, or a functional equivalent or a homolog thereof as depicted in column 8 of Table II, application no. 20, preferably a homolog or functional equivalent as depicted in column 8 of Table II B, application no. 20, and being depicted in the same respective line as said Yhr022c-a, and preferably the activity is increased non-targeted, whereby the respective line disclose in column 7 the fine chemical hentriacontane.

[102322] Accordingly, in one embodiment, the molecule which activity is to be increased in the process of the invention is the gene product with an activity as a “YHR022C-A-protein”, preferably it is the molecule of section (a) or (b) of this paragraph.

[102323] In particular, it was observed that in plants, especially in Arabidopsis thaliana, increasing or generating the activity of a gene product non-targeted with the activity of a “YHR022C-A-protein”, preferably being encoded by a gene comprising the nucleic acid sequence SEQ ID NO. 157858, preferably the coding region thereof, conferred the production of or the increase in hentriacontane compared with the wild type control.

[102324] The nucleic acid sequence of Yjl127w-a from Saccharomyces cerevisiae, e.g. as shown in the respective line in column 5 of Table I, application no. 20, has been published in A. And the activity of the gene product thereof is the activity of YJL127W-A-protein.

[102325] Accordingly, in one embodiment, the process of the present invention for producing hentriacontane in a non-human organism, like a microorganism or a plant or a part thereof, comprises increasing or generating the activity of a gene product with the activity of a gene product conferring the activity of “YJL127W-A-protein”, especially from Saccharomyces cerevisiae or its functional equivalent or its homolog, e.g. the increase of
[102326] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said Yjl127w-a, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said Yjl127w-a, and preferably the activity is increased non-targeted, or
[102327] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said Yjl127w-a, or a functional equivalent or a homolog thereof as depicted in column 8 of Table II, application no. 20, preferably a homolog or functional equivalent as depicted in column 8 of Table II B, application no. 20, and being depicted in the same respective line as said Yjl127w-a, and preferably the activity is increased non-targeted, whereby the respective line disclose in column 7 the fine chemical hentriacontane.

[102328] Accordingly, in one embodiment, the molecule which activity is to be increased in the process of the invention is the gene product with an activity as a “YJL127W-A-protein”, preferably it is the molecule of section (a) or (b) of this paragraph.

[102329] In particular, it was observed that in plants, especially in Arabidopsis thaliana, increasing or generating the activity of a gene product non-targeted with the activity of a “YJL127W-A-protein”, preferably being encoded by a gene comprising the nucleic acid sequence SEQ ID NO. 97631, preferably the coding region thereof, conferred the production of or the increase in hentriacontane compared with the wild type control.

[102330] The nucleic acid sequence of Ylr157c from Saccharomyces cerevisiae, e.g. as shown in the respective line in column 5 of Table I, application no. 20, has been published in A. And the activity of the gene product thereof is the activity of ylr157c-protein.

[102331] Accordingly, in one embodiment, the process of the present invention for producing hentriacontane in a non-human organism, like a microorganism or a plant or a part thereof, comprises increasing or generating the activity of a gene product with the activity of a gene product conferring the activity of “yid 57c-protein”, especially from Saccharomyces cerevisiae or its functional equivalent or its homolog, e.g. the increase of
[102332] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said Ylr157c, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said Ylr157c, and preferably the activity is increased plastidic, or
[102333] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said Ylr157c, or a functional equivalent or a homolog thereof as depicted in column 8 of Table II, application no. 20, preferably a homolog or functional equivalent as depicted in column 8 of Table II B, application no. 20, and being depicted in the same respective line as said Ylr157c, and preferably the activity is increased plastidic, whereby the respective line disclose in column 7 the fine chemical hentriacontane.

[102334] Accordingly, in one embodiment, the molecule which activity is to be increased in the process of the invention is the gene product with an activity as a “ylr157c-protein”, preferably it is the molecule of section (a) or (b) of this paragraph.

[102335] In particular, it was observed that in plants, especially in Arabidopsis thaliana, increasing or generating the activity of a gene product plastidic with the activity of a “yid 57c-protein”, preferably being encoded by a gene comprising the nucleic acid sequence SEQ ID NO. 157862, preferably the coding region thereof, conferred the production of or the increase in hentriacontane compared with the wild type control.

[102336] The nucleic acid sequence of Ynl321w from Saccharomyces cerevisiae, e.g. as shown in the respective line in column 5 of Table I, application no. 20, has been published in A. And the activity of the gene product thereof is the activity of ynl321w-protein.

[102337] Accordingly, in one embodiment, the process of the present invention for producing hentriacontane in a non-human organism, like a microorganism or a plant or a part thereof, comprises increasing or generating the activity of a gene product with the activity of a gene product conferring the activity of “ynl321w-protein”, especially from Saccharomyces cerevisiae or its functional equivalent or its homolog, e.g. the increase of
[102338] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said Ynl321w, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said Ynl321w, and preferably the activity is increased plastidic, or
[102339] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said Ynl321w, or a functional equivalent or a homolog thereof as depicted in column 8 of Table II, application no. 20, preferably a homolog or functional equivalent as depicted in column 8 of Table II B, application no. 20, and being depicted in the same respective line as said Ynl321w, and preferably the activity is increased plastidic, whereby the respective line disclose in column 7 the fine chemical hentriacontane.

[102340] Accordingly, in one embodiment, the molecule which activity is to be increased in the process of the invention is the gene product with an activity as a “ynl321w-protein”, preferably it is the molecule of section (a) or (b) of this paragraph.

[102341] In particular, it was observed that in plants, especially in Arabidopsis thaliana, increasing or generating the activity of a gene product plastidic with the activity of a “ynl321w-protein”, preferably being encoded by a gene comprising the nucleic acid sequence SEQ ID NO. 129546, preferably the coding region thereof, conferred the production of or the increase in hentriacontane compared with the wild type control.

[102342] The nucleic acid sequence of Ypr170c from Saccharomyces cerevisiae, e.g. as shown in the respective line in column 5 of Table I, application no. 20, has been published in A. And the activity of the gene product thereof is the activity of ypr170c-protein.

[102343] Accordingly, in one embodiment, the process of the present invention for producing hentriacontane in a non-human organism, like a microorganism or a plant or a part thereof, comprises increasing or generating the activity of a gene product with the activity of a gene product conferring the activity of “ypr170c-protein”, especially from Saccharomyces cerevisiae or its functional equivalent or its homolog, e.g. the increase of
[102344] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said Ypr170c, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said Ypr170c, and preferably the activity is increased plastidic, or
[102345] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said Ypr170c, or a functional equivalent or a homolog thereof as depicted in column 8 of Table II, application no. 20, preferably a homolog or functional equivalent as depicted in column 8 of Table II B, application no. 20, and being depicted in the same respective line as said Ypr170c, and preferably the activity is increased plastidic, whereby the respective line disclose in column 7 the fine chemical hentriacontane.

[102346] Accordingly, in one embodiment, the molecule which activity is to be increased in the process of the invention is the gene product with an activity as a “ypr170c-protein”, preferably it is the molecule of section (a) or (b) of this paragraph.

[102347] In particular, it was observed that in plants, especially in Arabidopsis thaliana, increasing or generating the activity of a gene product plastidic with the activity of a “ypr170c-protein”, preferably being encoded by a gene comprising the nucleic acid sequence SEQ ID NO. 113647, preferably the coding region thereof, conferred the production of or the increase in hentriacontane compared with the wild type control.

[102348] The nucleic acid sequence of YPR172W from Saccharomyces cerevisiae, e.g. as shown in the respective line in column 5 of Table I, application no. 20, has been published in A. And the activity of the gene product thereof is the activity of YPR172W-protein.

[102349] Accordingly, in one embodiment, the process of the present invention for producing hentriacontane in a non-human organism, like a microorganism or a plant or a part thereof, comprises increasing or generating the activity of a gene product with the activity of a gene product conferring the activity of “YPR172W-protein”, especially from Saccharomyces cerevisiae or its functional equivalent or its homolog, e.g. the increase of
[102350] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said YPR172W, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said YPR172W, and preferably the activity is increased non-targeted, or
[102351] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said YPR172W, or a functional equivalent or a homolog thereof as depicted in column 8 of Table II, application no. 20, preferably a homolog or functional equivalent as depicted in column 8 of Table II B, application no. 20, and being depicted in the same respective line as said YPR172W, and preferably the activity is increased non-targeted, whereby the respective line disclose in column 7 the fine chemical hentriacontane.

[102352] Accordingly, in one embodiment, the molecule which activity is to be increased in the process of the invention is the gene product with an activity as a “YPR172W-protein”, preferably it is the molecule of section (a) or (b) of this paragraph.

[102353] In particular, it was observed that in plants, especially in Arabidopsis thaliana, increasing or generating the activity of a gene product non-targeted with the activity of a “YPR172W-protein”, preferably being encoded by a gene comprising the nucleic acid sequence SEQ ID NO. 158033, preferably the coding region thereof, conferred the production of or the increase in hentriacontane compared with the wild type control.

[102354] The nucleic acid sequence of B3644 from Escherichia coli, e.g. as shown in the respective line in column 5 of Table I, application no. 20, has been published in B. And the activity of the gene product thereof is the activity of b3644-protein.

[102355] Accordingly, in one embodiment, the process of the present invention for producing hentriacontane in a non-human organism, like a microorganism or a plant or a part thereof, comprises increasing or generating the activity of a gene product with the activity of a gene product conferring the activity of “b3644-protein”, especially from Escherichia coli or its functional equivalent or its homolog, e.g. the increase of
[102356] (a) a gene product of a gene comprising the nucleic acid molecule as shown in the respective line in column 5 of Table I (whereby the respective line disclose in column 7 the fine chemical hentriacontane), application no. 20, preferably the coding region thereof, or a homolog or a fragment thereof, and being depicted in the same respective line as said B3644, or a functional equivalent or a homolog thereof as shown in column 8 of Table I, application no. 20, preferably the coding region thereof, particularly a homolog or functional equivalent as shown in column 8 of Table I B, application no. 20, and being depicted in the same respective line as said B3644, and preferably the activity is increased non-targeted, or
[102357] (b) a polypeptide comprising a polypeptide, a consensus sequence or at least a polypeptide motif as shown in the respective line in column 5 of Table II or in column 8 of Table IV, application no. 20, respectively, and being depicted in the same respective line as said B3644, or a functional equivalent or a homolog thereof as depicted in column 8 of Table II, application no. 20, preferably a homolog or functional equivalent as depicted in column 8 of Table II B, application no. 20, and being depicted in the same respective line as said B3644, and preferably the activity is increased non-targeted, whereby the respective line disclose in column 7 the fine chemical hentriacontane.

[102358] Accordingly, in one embodiment, the molecule which activity is to be increased in the process of the invention is the gene product with an activity as a “b3644-protein”, preferably it is the molecule of section (a) or (b) of this paragraph.

[102359] In particular, it was observed that in plants, especially in Arabidopsis thaliana, increasing or generating the activity of a gene product non-targeted with the activity of a “b3644-protein”, preferably being encoded by a gene comprising the nucleic acid sequence SEQ ID NO. 125348, preferably the coding region thereof, conferred the production of or the increase in hentriacontane compared with the wild type control.

[102360] for the disclosure of this paragraph see [0105.1.7.7] above

[102361] for the disclosure of this paragraph see [0106.1.7.7] above

[102362] for the disclosure of this paragraph see [0107.1.7.7] above

[102363] A protein having an activity conferring an increase in the amount or level of the fine chemical hentriacontane, upon targeting to the plastids or mitochondria or upon non-targeting, preferably has the structure of the respective polypeptide described herein, in particular of the polypeptides comprising the consensus sequence or at least one polypeptide motifs as shown in the respective line in Table IV, application no. 20, column 8 or of the polypeptide comprising an amino acid sequence as disclosed in the respective line in Table II, application no. 20, columns 5 or 8, or homologs or fragments thereof as described herein, or is encoded by the nucleic acid molecule characterized herein or the nucleic acid molecule according to the invention, for example by the nucleic acid molecule as shown in the respective line in Table I, application no. 20, columns 5 or 8, preferably the coding region thereof, or homologs or fragments thereof and has the herein mentioned activity.

[102364] for the disclosure of this paragraph see [0109.1.7.7] above

[102365] for the disclosure of this paragraph see [0110.1.7.7] above

[102366] In one embodiment, the process of the present invention comprises one or more of the following steps
[102367] (a) stabilizing a protein conferring the generation or increased expression of a protein encoded by the nucleic acid molecule of the invention or of the polypeptide of the invention, e.g. of a polypeptide having the activity selected from the group consisting of 3-deoxy-7-phosphoheptulonate synthase, acid shock protein, amino-acid acetyltransferase, assembly protein for periplasmic nitrate reductase, At2g45420-protein, At5g43630-protein, ATP-dependent RNA helicase, b0833-protein, b1259-protein, b1445-protein, b1670-protein, b1930-protein, b2399-protein, b2653-protein, b3644-protein, cell division protein, cyclin D, cysteine synthase A, dihydroxyacetone kinase, electron transport complex protein, ethanolamine utilization protein, exonuclease subunit, fumarylacetoacetate hydrolase, GDP-mannose 4,6-dehydratase, inner membrane protein, ion-transport protein, L-asparaginase, MADS box protein transcription factor, mechanosensitive ion channel, metal-dependent hydrolase, methionine permease, O-antigen chain length determinant, ornithine decarboxylase, orotidine-5′-phosphate decarboxylase, peptide chain release factor, phospho-2-dehydro-3-deoxyheptonate aldolase, phosphoadenosine phosphosulfate reductase, phosphoglycolate phosphatase, pyrimidine-specific ribonucleoside hydrolase, pyruvate kinase, RNA-binding protein, S-adenosyl-L-methionine-dependent methyltransferase, serine protease, short-chain alcohol dehydrogenase family, sigma-54 dependent response regulator, spermidine synthase, sugar transport system permease protein, transcription factor, transcriptional regulator, YFL019C-protein, YHL039W-protein, YHR022C-A-protein, YJL127W-A-protein, ylr157c-protein, ynl321w-protein, ypr170c-protein, and YPR172W-protein, or of a polypeptide as indicated in the respective line in Table II, application no. 20, columns 5 or 8, or its homologs or fragments, and conferring the production of or an increase in hentriacontane, respectively; and/or
[102368] (b) stabilizing a mRNA conferring the generation or increased expression of a FCRP, e.g. protein encoded by the nucleic acid molecule of the invention or its homologs or fragments, or of a mRNA encoding the polypeptide of the present invention having the hereinmentioned activity selected from the group consisting of said activities mentioned in (a) and conferring the production of or an increase in hentriacontane, respectively; and/or
[102369] (c) increasing the specific activity of a protein conferring the increased expression of a FCRP, e.g. a protein encoded by the nucleic acid molecule of the invention or of the polypeptide of the present invention having herein-mentioned hentriacontane generating or increasing activity, respectively, e.g. of a polypeptide having the activity of a protein as indicated in the respective line in Table II, application no. 20, columns 5 or 8, or its homologs or fragments, or decreasing the inhibitory regulation of the polypeptide of the invention; and/or
[102370] (d) generating or increasing the expression of an endogenous or artificial transcription factor mediating the expression of a protein conferring the generation or increased expression of a FCRP, e.g. a protein encoded by the nucleic acid molecule of the invention or of the polypeptide of the invention having herein-mentioned activity selected from the group consisting of said activities mentioned in (a) and conferring a hentriacontane increasing activity, respectively, e.g. of a polypeptide having the activity of a protein as indicated in the respective line in Table II, application no. 20, columns 5 or 8 or its homologs; and/or
[102371] (e) stimulating activity of a protein conferring the increased expression of a FCRP, e.g. a protein encoded by the nucleic acid molecule of the present invention or a polypeptide of the present invention having herein-mentioned activity selected from the group consisting of said activities mentioned in (a) and conferring a hentriacontane increasing activity, respectively, e.g. of a polypeptide having the activity of a protein as indicated in the respective line in Table II, application no. 20, columns 5 or 8, or its homologs or fragments, by adding one or more exogenous inducing factors to the non-human organism or parts thereof; and/or
[102372] (f) expressing a transgenic gene encoding a protein conferring the increased expression of a FCRP, e.g. a polypeptide encoded by the nucleic acid molecule of the present invention or a polypeptide of the present invention, having herein-mentioned activity selected from the group consisting of said activities mentioned in (a) and conferring a hentriacontane increasing activity, respectively, e.g. of a polypeptide having the activity of a protein as indicated in the respective line in Table II, application no. 20, columns 5 or 8, or its homologs or fragments, and/or
[102373] (g) increasing the copy number of a gene conferring the increased expression of a nucleic acid molecule encoding a FCRP, e.g. a polypeptide encoded by the nucleic acid molecule of the invention or the polypeptide of the invention having herein-mentioned activity selected from the group consisting of said activities mentioned in (a) and conferring a hentriacontane increasing activity, respectively, e.g. of a polypeptide having the activity of a protein as indicated in the respective line in Table II, application no. 20, columns 5 or 8, or its homologs or fragments; and/or
[102374] (h) increasing the expression of the endogenous gene encoding the FCRP, polypeptide of the invention, e.g. a polypeptide encoded by the nucleic acid molecule of the invention or the polypeptide of the invention having herein-mentioned activity selected from the group consisting of said activities mentioned in (a) and conferring a hentriacontane; increasing activity, respectively, e.g. of a polypeptide having the activity of a protein as indicated in the respective line in Table II, application no. 20, columns 5 or 8, or its homologs or fragments, by adding positive expression or removing negative expression elements, e.g. homologous recombination can be used to either introduce positive regulatory elements like for plants the 35S enhancer into the promoter or to remove repressor elements form regulatory regions. Further gene conversion methods can be used to disrupt repressor elements or to enhance to activity of positive elements. Positive elements can be randomly introduced in plants by T-DNA or transposon mutagenesis and lines can be identifled in which the positive elements have be integrated near to a gene of the invention, the expression of which is thereby enhanced; and/or
[102375] (i) modulating growth conditions of the non-human organism in such a manner, that the expression or activity of the gene encoding the FCRP, e.g. a protein of the invention or the protein itself is enhanced, for example microorganisms or plants can be grown for example under a higher temperature regime leading to an enhanced expression of heat shock proteins, which can lead to an enhanced hentriacontane production, respectively; and/or
[102376] (j) selecting of non-human organisms with especially high activity of the proteins of the invention from natural or from mutagenized resources and breeding them into the target organisms, e.g. the elite crops; and/or
[102377] (k) directing a protein encoded by the nucleic acid molecule of the invention or of the polypeptide of the present invention having herein-mentioned activity selected from the group consisting of said activities mentioned in (a) and conferring a hentriacontane increasing activity, respectively, e.g. of a polypeptide having the activity of a protein as indicated in the respective line in Table II, application no. 20, columns 5 or 8, or its homologs or fragments, if for the corresponding nucleic acid molecule in the respective line in column 6 of Table I the term “plastidic” is indicated, to the plastids by the addition of a plastidial targeting sequence or if for the corresponding nucleic acid molecule in the respective line in column 6 of Table I the term “mitochondric” is indicated, to the mitochondria by the addition of a mitochondrial targeting sequence; and/or
[102378] (l) generating the expression of a protein encoded by the nucleic acid molecule of the invention or of the polypeptide of the present invention having herein-mentioned activity selected from the group consisting of said activities mentioned in (a) and conferring a hentriacontane increasing activity, respectively, e.g. of a polypeptide having the activity of a protein as indicated in the respective line in Table II, application no. 20, columns 5 or 8, or its homologs or fragments, if for the corresponding nucleic acid molecule in the respective line in column 6 of Table I the term “plastidic” or “mitochondric” is indicated, in these organelles by the stable or transient transformation, advantageously stable transformation, of organelles, preferably plastids or mitochondria, with an inventive nucleic acid sequence preferably in form of an expression cassette containing said sequence leading to the expression of the nucleic acids or polypeptides of the invention in the respective organelle; and/or
[102379] (m) generating the expression of a protein encoded by the nucleic acid molecule of the invention or of the polypeptide of the present invention having herein-mentioned activity selected from the group consisting of said activities mentioned in (a) and conferring a hentriacontane increasing activity, respectively, e.g. of a polypeptide having the activity of a protein as indicated in the respective line in Table II, application no. 20, columns 5 or 8, or its homologs or fragments, if for the corresponding nucleic acid molecule in the respective line in column 6 of Table I the term “plastidic” or “mitochondric” is indicated, in these organelles by integration of a nucleic acid of the invention into the genome of the respective organelle under control of preferable a promoter selective for the respective organelle.

[102380] Preferably, said mRNA is the coding region of the nucleic acid molecule of the present invention and/or the protein conferring the increased expression of a protein encoded by the coding region of the nucleic acid molecule of the present invention alone or linked to a transit nucleic acid sequence or transit peptide encoding nucleic acid sequence or the polypeptide having the herein mentioned activity, e.g. conferring the generation of or increase of hentriacontane, respectively, after increasing the expression or activity of the encoded polypeptide, non-targeted or in organelles such as plastids and/or mitochondria, preferably plastids, or having the activity of a polypeptide having an activity as the protein as shown in the respective line in Table II, application no. 20, column 3, or its homologs. Preferably the increase of hentriacontane, respectively, takes place non-targeted or in plastids and/or mitochondria, preferably non-targeted or in plastids.

[102381] [0113.1.7.20] to [0122.1.7.20] for the disclosure of these paragraphs see [0113.1.7.7] to [0122.1.7.7] above

[102382] The activation of an endogenous polypeptide having above-mentioned activity, e.g. having the activity of a protein as shown in the respective line in Table II, application no. 20, column 3 or of the polypeptide of the invention, e.g. conferring the increase of the fine chemical hentriacontane, respectively, by increase of expression or activity in the cytoplasm, and/or in the cytosol, and/or in an organelle, such as plastids or mitochondria, can also be increased by introducing a synthetic transcription factor, which binds close to the coding region of the gene encoding the protein as shown in the respective line in Table II, application no. 20, column 5 or 8, or homologs or fragments thereof, and activates its transcription. A chimeric zinc finger protein can be constructed, which comprises a specific DNA-binding domain and an activation domain as e.g. the VP16 domain of Herpes Simplex virus. The specific binding domain can bind to the regulatory region of the gene encoding the protein as shown in the respective line in Table II, application no. 20, column 5 or 8, or homologs or fragments thereof. The expression of the chimeric transcription factor in a non-human organism, in particular in a plant, leads to a specific expression of the protein as shown in the respective line in Table II, application no. 20, column 5 or 8, or homologs or fragments thereof, see e.g. in WO01/52620, Oriz, Proc. Natl. Acad. Sci. USA 99, 13290 (2002), or Guan, Proc. Natl. Acad. Sci. USA 99, 13296 (2002).

[102383] [0124.1.7.20] to [0127.1.7.20] for the disclosure of these paragraphs see [0124.1.7.7] to [0127.1.7.7] above

[102384] Owing to the introduction of a gene or a plurality of genes conferring the expression of the nucleic acid molecule of the invention or the polypeptide of the invention, for example the nucleic acid construct mentioned below, or encoding the protein as shown in the respective line in Table II, application no. 20, column 5 or 8, or homologs or fragments thereof, into a non-human organism alone or in combination with other genes, it is possible not only to increase the biosynthetic flux towards the end product, but also to increase, modify or create de novo an advantageous, preferably novel metabolites composition in the non-human organism, e.g. an advantageous composition comprising a higher content of (from a viewpoint of nutritional physiology limited) hentriacontane and if desired other long chain n-alkanes (C20 to C40) or derivates like alcohols and acid of alkanes, optionally unsaturated as well as break down and/or cracking products of long chain alkanes, or waxes or components thereof, and/or other metabolites, in free or bound form.

[102385] for the disclosure of this paragraph see [0129.1.7.7] above

[102386] Accordingly, in one embodiment, the process according to the invention relates to a process, which comprises:
[102387] (a) providing a non-human organism, preferably a microorganism, a plant cell, a plant tissue, a plant or a part thereof, more preferably a microorganism, a plant tissue, a plant or a part thereof;
[102388] (b) increasing the activity of a protein as shown in the respective line in Table II, application no. 20, column 3 or of a polypeptide being encoded by the respective nucleic acid molecule of the present invention and described below, e.g. conferring an increase of the fine chemical hentriacontane, respectively, in the non-human organism, preferably in the microorganism, the plant cell, the plant tissue, the plant or a part thereof, more preferably a microorganism, a plant tissue, a plant or a part thereof, especially cytoplasmic or in an organelle, like plastids or mitochondria,
[102389] (c) growing the non-human organism, preferably the microorganism, the plant cell, the plant tissue, the plant or a part thereof, under conditions which permit the production of said chemical in the non-human organism, preferably the microorganism, the plant cell, the plant tissue, the plant or a part thereof; and
[102390] (d) if desired, recovering, optionally isolating, said fine chemical, in free and/or bound form, and, optionally further free and/or bound long chain n-alkanes (C20 to C40) or derivates like alcohols and acid of alkanes, optionally unsaturated as well as break down and/or cracking products of long chain alkanes or waxes or components thereof, and/or other metabolites synthesized by the non-human organism, the microorganism, the plant cell, the plant tissue, the plant or a part thereof.

[102391] [0131.1.7.20] to [0133.1.7.20] for the disclosure of these paragraphs see [0131.1.7.7] to [0133.1.7.7] above

[102392] The non-human organism, in particular the microorganism, plant or part thereof, is advantageously grown in such a way that it is not only possible to recover, if desired to isolate the free or bound fine chemical or the free and bound fine chemical but as option it is also possible to produce, recover and, if desired to isolate, other free or/and bound long chain n-alkanes (C20 to C40) or derivates like alcohols and acid of alkanes, optionally unsaturated as well as break down and/or cracking products of long chain alkanes or waxes or components thereof and/or other metabolites.

[102393] The organisms are harvested in a manner known per se and appropriate for the particular organism. Microorganisms such as bacteria, mosses, yeasts and fungi or plant cells which are cultured in liquid media by fermentation may be removed, for example, by centrifugation, decanting or filtration. Plants are grown on solid media in a manner known per se and harvested accordingly.

[102394] [0135.1.7.20] to [0139.1.7.20] for the disclosure of these paragraphs see [0135.1.7.7] to [0139.1.7.7] above

[102395] In another embodiment, the invention relates to a (isolated) nucleic acid molecule, which comprises a nucleic acid molecule selected from the group consisting of:
[102396] (a) a nucleic acid molecule encoding a polypeptide shown in Table II, application no. 20, column 5 or 8, preferably shown in Table II B, application no. 20, column 8, or a homolog or a fragment thereof;
[102397] (b) a nucleic acid molecule shown in Table I, application no. 20, column 5 or 8, preferably shown in Table I B, application no. 20, column 8, preferably the coding region thereof, or a homolog or a fragment thereof;
[102398] (c) a nucleic acid molecule, which, as a result of the degeneracy of the genetic code, can be derived from a polypeptide sequence depicted in column 5 or 8 of Table II, application no. 20, preferably in column 8 of Table II B, application no. 20;
[102399] (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 20, preferably shown in column 8 of Table I B, application no. 20, preferably the coding region thereof;
[102400] (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
[102401] (f) a nucleic acid molecule which hybridizes with a nucleic acid molecule of (a), (b), (c), (d) or (e) under stringent hybridization conditions;
[102402] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
[102403] (h) a nucleic acid molecule encoding a polypeptide comprising the consensus sequence or one or more polypeptide motifs as shown in column 8 of Table IV, application no. 20;
[102404] (i) a nucleic acid molecule encoding a polypeptide having the activity represented by a respective protein as depicted in column 5 of Table II, application no. 20;
[102405] (j) a nucleic acid molecule comprising a polynucleotide which is obtained by amplifying a cDNA library or a genomic library using the primers in Table III, application no. 20, column 8;
[102406] and
[102407] (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
[102408] or a nucleic acid molecule comprising a sequence which is complementary thereto.
   (All the references to Tables I to IV are references to the corresponding lines of Tables I to IV.)

[102409] In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 20.

[102410] In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[102411] In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II application no. 20 and confers a generation or an increase of the respective fine chemical in a non-human organism or a part thereof as compared to a corresponding wild-type non-human organism or part thereof.

[102412] In another embodiment, the invention relates to a (isolated) nucleic acid molecule, which comprises a nucleic acid molecule selected from the group consisting of:
[102413] (a) a nucleic acid molecule encoding a polypeptide shown in Table II, column 5 or 8, application no. 20, preferably shown in Table II A, application no. 20, in column 5 or in Table II A, application no. 20, column 8 or in Table II B, application no. 20, column 8, or a homolog or a fragment thereof;
[102414] (b) a nucleic acid molecule shown in Table I, application no. 20, column 5 or 8, preferably shown in Table I A, application no. 20, in column 5 or in Table I A, application no. 20, column 8 or in Table I B, application no. 20, column 8, preferably the coding region thereof, or a homolog or a fragment thereof;
[102415] (c) a nucleic acid molecule, which, as a result of the degeneracy of the genetic code, can be derived from a polypeptide sequence depicted in column 5 or 8 of Table II, application no. 20, preferably shown in Table II A, application no. 20, in column 5 or in Table II A, application no. 20, column 8 or in Table II B, application no. 20, column 8;
[102416] (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 20, preferably shown in Table I A, application no. 20, in column 5 or in Table I A, application no. 20, column 8 or in Table I B column 8, preferably the coding region thereof;
[102417] (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
[102418] (f) a nucleic acid molecule which hybridizes with a nucleic acid molecule of (a), (b), (c), (d) or (e) under stringent hybridization conditions;
[102419] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
[102420] (h) a nucleic acid molecule encoding a polypeptide comprising the consensus sequence or one or more polypeptide motifs as shown in column 8 of Table IV, application no. 20;
[102421] (i) a nucleic acid molecule encoding a polypeptide having the activity represented by a respective protein as depicted in column 5 of Table II, application no. 20;
[102422] (j) a nucleic acid molecule comprising a polynucleotide which is obtained by amplifying a cDNA library or a genomic library using the primers in Table III, application no. 20, column 8;
[102423] and
[102424] (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt,500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
[102425] or a nucleic acid molecule comprising a sequence which is complementary thereto;
   whereby, preferably, the nucleic acid molecule according to (a), (b), (c), (d), (e), (f), (g), (h), (i), (j) or (k) distinguishes over the sequence depicted in Table I A and/or I B, application no. 20, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 20, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 20, column 5 or 8, or the coding region thereof, respectively. In a further embodiment the nucleic acid molecule does not encode the polypeptide sequence shown in Table II A and/or II B, application no. 20, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 20, column 5 or 8 and does not encode a protein of the sequence shown in Table II A and/or II B, application no. 20, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 20, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 20, column 5 or 8 but less than 100%, preferably less than 99.999%, 99.99%, 99.9%, 99%, 98%, 97%, 96% or 95% identical to the sequence shown in Table II A and/or II B, application no. 20, column 5 or 8.
   (All the references to Tables I to IV are references to the corresponding lines of Tables I to IV.)

[102426] In a preferred embodiment thereof said nucleic acid molecule according to (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 20.

[102427] In another preferred embodiment thereof said nucleic acid molecule according to (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[102428] In another preferred embodiment thereof said nucleic acid molecule according to (a), (b), (c), (d), (e), (f), (g), (h), (i), (j) or (k) encodes a polypeptide which has the activity of the respective polypeptide represented by a protein as depicted in the respective line in column 5 of Table II, application no. 20, and confers a generation or an increase of the respective fine chemical in a non-human organism or a part thereof as compared to a corresponding wild-type non-human organism or part thereof.

[102429] [0144.1.7.20] to [0155.1.7.20] for the disclosure of these paragraphs see [0144.1.7.7] to [0155.1.7.7] above

[102430] In a preferred embodiment a nucleic acid construct, for example an expression cassette, comprises upstream, i.e. at the 5′ end of the encoding sequence, a promoter and downstream, i.e. at the 3′ end, a polyadenylation signal and optionally other regulatory elements which are operably linked to the intervening encoding sequence with one of the nucleic acids as depicted in the respective line in Table I, application no. 20, column 5 or 8, preferably the coding region thereof, or a homolog or a fragment thereof. By an operable linkage is meant the sequential arrangement of promoter, encoding sequence, terminator, optionally other regulatory elements and optionally targeting sequences in such a way that each of the regulatory elements/targeting sequence can fulfill its function in the expression of the encoding sequence in due manner. In one embodiment the sequences preferred for operable linkage are targeting sequences for ensuring subcellular localization in organelles, like plastids or mitochondria. However, targeting sequences for ensuring subcellular localization in the endoplasmic reticulum (=ER), in the nucleus, in oil corpuscles or other compartments may also be employed as well as translation promoters such as the 5′ lead sequence in tobacco mosaic virus (Gallie et al., Nucl. Acids Res. 15, 8693 (1987)).

[102431] for the disclosure of this paragraph see [0157.1.7.7] above

[102432] In an embodiment, the invention relates to an expression cassette, comprising
[102433] 1) a promoter, preferably a promoter selected from the group consisting of Big35S, PCUbi, Super and USP;
[102434] 2) a nucleic acid molecule selected from the group consisting of
   [102435] (a) a nucleic acid molecule encoding a polypeptide shown in Table II, application no. 20, column 5 or 8, preferably shown in Table II A, application no. 20, column 5, or in Table II A, application no. 20, column 8, or in Table II B, application no. 20, column 8, or a homolog or a fragment thereof;
   [102436] (b) a nucleic acid molecule shown in Table I, application no. 20, column 5 or 8, preferably shown in Table I A, application no. 20, column 5, or in Table I A, application no. 20, column 8, or in Table I B, application no. 20, column 8, preferably the coding region thereof, or a homolog or a fragment thereof;
   [102437] (c) a nucleic acid molecule, which, as a result of the degeneracy of the genetic code, can be derived from a polypeptide sequence depicted in column 5 or 8 of Table II, application no. 20, preferably shown in Table II A, application no. 20, column 5, or in Table II A, application no. 20, column 8, or in Table II B, application no. 20, column 8;
   [102438] (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 20, preferably shown in Table I A, application no. 20, column 5, or in Table I A, application no. 20, column 8, or in Table I B, application no. 20, column 8, preferably the coding region thereof;
   [102439] (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   [102440] (f) a nucleic acid molecule which hybridizes with a nucleic acid molecule of (a), (b), (c), (d) or (e) under stringent hybridization conditions;
   [102441] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   [102442] (h) a nucleic acid molecule encoding a polypeptide comprising the consensus sequence or one or more polypeptide motifs as shown in column 8 of Table IV, application no. 20;
   [102443] (i) a nucleic acid molecule encoding a polypeptide having the activity represented by a respective protein as depicted in column 5 of Table II, application no. 20;
   [102444] (j) a nucleic acid molecule comprising a polynucleotide which is obtained by amplifying a cDNA library or a genomic library using the primers in Table III, application no. 20, column 8;
   [102445] and
   [102446] (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   [102447] or a nucleic acid molecule comprising a sequence which is complementary thereto.
      and, optionally
[102448] 3) nucleic acid molecule encoding a transit peptide, preferably encoding a plastidal transit peptide or a mitochondrial transit peptide;
   which are operable linked.
   (All the references to Tables I to IV are references to the corresponding lines of Tables I to IV.)

[102449] In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 20.

[102450] In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[102451] In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 20 and confers a generation or an increase of the respective fine chemical in a non-human organism or a part thereof as compared to a corresponding wild-type non-human organism or part thereof.

[102452] In another embodiment thereof the expression cassette comprises 3) a nucleic acid molecule encoding a transit peptide, preferably encoding a plastidal transit peptide, especially in case in column 6 of Table I, application no. 20, in the same line as of the respective nucleic acid molecule according to 2) “plastidic” is depicted, or encoding a mitochondrial transit peptide, especially in case in column 6 of Table I, application no. 20, in the same line as of the respective nucleic acid molecule according to 2) “mitochondric” is depicted.

[102453] In another embodiment thereof the expression cassette does not comprise 3) a nucleic acid molecule encoding a transit peptide, especially in case in column 6 of Table I, application no. 20, in the same line as of the respective nucleic acid molecule according to 2) “non-targeted” is depicted.

[102454] In a further embodiment, the invention relates to an expression cassette, comprising
[102455] 1) a promoter, preferably a promoter selected from the group consisting of Big35S, PCUbi, Super and USP;
[102456] 2) a nucleic acid molecule selected from the group consisting of
   [102457] (a) a nucleic acid molecule encoding a polypeptide shown in Table II, application no. 20, column 5 or 8, preferably shown in Table II A, application no. 20, column 5, or in Table II A, application no. 20, column 8, or in Table II B, application no. 20, column 8, or a homolog or a fragment thereof;
   [102458] (b) a nucleic acid molecule shown in Table I, application no. 20, column 5 or 8, preferably shown in Table I A, application no. 20, column 5, or in Table I A, application no. 20, column 8, or in Table I B, application no. 20, column 8, preferably the coding region thereof, or a homolog or a fragment thereof;
   [102459] (c) a nucleic acid molecule, which, as a result of the degeneracy of the genetic code, can be derived from a polypeptide sequence depicted in column 5 or 8 of Table II, application no. 20, preferably shown in Table II A, application no. 20, column 5, or in Table II A, application no. 20, column 8, or in Table II B, application no. 20, column 8;
   [102460] (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 20, preferably shown in Table I A, application no. 20, column 5, or in Table I A, application no. 20, column 8,or in Table I B, application no. 20, column 8, preferably the coding region thereof;
   [102461] (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   [102462] (f) a nucleic acid molecule which hybridizes with a nucleic acid molecule of (a), (b), (c), (d) or (e) under stringent hybridization conditions;
   [102463] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   [102464] (h) a nucleic acid molecule encoding a polypeptide comprising the consensus sequence or one or more polypeptide motifs as shown in column 8 of Table IV, application no. 20;
   [102465] (i) a nucleic acid molecule encoding a polypeptide having the activity represented by a respective protein as depicted in column 5 of Table II, application no. 20;
   [102466] (j) a nucleic acid molecule comprising a polynucleotide which is obtained by amplifying a cDNA library or a genomic library using the primers in Table III, application no. 20, column 8;
   [102467] and
   [102468] (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   [102469] or a nucleic acid molecule comprising a sequence which is complementary thereto;
      and, optionally
[102470] 3) nucleic acid molecule encoding a transit peptide, preferably encoding a plastidal transit peptide or a mitochondrial transit peptide;
   which are operable linked and
   whereby, preferably, the nucleic acid molecule according to 2 (a), (b), (c), (d), (e), (f), (g), (h), (i), (j) or (k) distinguishes over the sequence depicted in Table I A and/or I B, application no. 20, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention according to 2) does not consist of the sequence shown in Table I A and/or I B, application no. 20, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 20, column 5 or 8, or the coding region thereof, respectively. In a further embodiment the nucleic acid molecule does not encode the polypeptide sequence shown in Table II A and/or II B, application no. 20, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 20, column 5 or 8 and does not encode a protein of the sequence shown in Table II A and/or II B, application no. 20, column 5 or 8. Accordingly, in one embodiment, the protein encoded by a sequence of a nucleic acid according to 2(a), (b), (c), (d), (e), (f), (g), (h), (i), (j) or (k) does not consist of the sequence shown in Table I A and/or I B, application no. 20, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 20, column 5 or 8 but less than 100%, 99.999%, 99.99%, 99.9%, 99%, 98%, 97%, 96% or 95% identical to the sequence shown in Table II A and/or II B, application no. 20, column 5 or 8.
   (All the references to Tables I to IV are references to the corresponding lines of Tables I to IV.)

[102471] In a preferred embodiment thereof said nucleic acid molecule according to 2 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 20.

[102472] In another preferred embodiment thereof said nucleic acid molecule according to 2 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[102473] In another preferred embodiment thereof said nucleic acid molecule according to 2 (a), (b), (c), (d), (e), (f), (g), (h), (i), (j) or (k) encodes a polypeptide which has the activity of the respective polypeptide represented by a protein as depicted in the respective line in column 5 of Table II application no. 20 and confers a generation or an increase of the respective fine chemical in a non-human organism or a part thereof as compared to a corresponding wild-type non-human organism or part thereof.

[102474] In another embodiment thereof the expression cassette comprises 3) a nucleic acid molecule encoding a transit peptide, preferably encoding a plastidal transit peptide, especially in case in column 6 of Table I, application no. 20, in the same line as of the respective nucleic acid molecule according to 2) “plastidic” is depicted, or encoding a mitochondrial transit peptide, especially in case in column 6 of Table I, application no. 20, in the same line as of the respective nucleic acid molecule according to 2) “mitochondric” is depicted.

[102475] In another embodiment thereof the expression cassette does not comprise 3) a nucleic acid molecule encoding a transit peptide, especially in case in column 6 of Table I, application no. 20, in the same line as of the respective nucleic acid molecule according to 2) “non-targeted” is depicted.

[102476] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 113714, or a homolog or fragment thereof, or a homolog thereof being depicted in Table I, application no. 20, column 8, in the same line as SEQ ID NO. 113714, preferably the coding region thereof, a homolog or a fragment thereof; which are operable linked.

[102477] In an embodiment thereof the expression cassette comprises in addition c) a nucleic acid molecule encoding a transit peptide, especially a plastidal transit petide in case in Table I, application no. 20, in column 6 in the same line as SEQ ID NO. 113714 is depicted, plastidic is mentioned, or a mitochondrial transit peptide in case in Table I, application no. 20, in column 6 in the same line as SEQ ID NO. 113714 is depicted, mitochondric is mentioned; which is operable linked, too.

[102478] In another embodiment thereof the expression cassette does not comprise an additional transit peptide in case in Table I, application no. 20, in column 6 in the same line as SEQ ID NO. 113714 is depicted, non-targeted is mentioned.

[102479] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 108727, or a homolog or fragment thereof, or a homolog thereof being depicted in Table I, application no. 20, column 8, in the same line as SEQ ID NO. 108727, preferably the coding region thereof, a homolog or a fragment thereof; which are operable linked.

[102480] In an embodiment thereof the expression cassette comprises in addition c) a nucleic acid molecule encoding a transit peptide, especially a plastidal transit petide in case in Table I, application no. 20, in column 6 in the same line as SEQ ID NO. 108727 is depicted, plastidic is mentioned, or a mitochondrial transit peptide in case in Table I, application no. 20, in column 6 in the same line as SEQ ID NO. 108727 is depicted, mitochondric is mentioned; which is operable linked, too.

[102481] In another embodiment thereof the expression cassette does not comprise an additional transit peptide in case in Table I, application no. 20, in column 6 in the same line as SEQ ID NO. 108727 is depicted, non-targeted is mentioned.

[102482] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 1623, or a homolog or fragment thereof, or a homolog thereof being depicted in Table I, application no. 20, column 8, in the same line as SEQ ID NO. 1623, preferably the coding region thereof, a homolog or a fragment thereof; which are operable linked.

[102483] In an embodiment thereof the expression cassette comprises in addition c) a nucleic acid molecule encoding a transit peptide, especially a plastidal transit petide in case in Table I, application no. 20, in column 6 in the same line as SEQ ID NO. 1623 is depicted, plastidic is mentioned, or a mitochondrial transit peptide in case in Table I, application no. 20, in column 6 in the same line as SEQ ID NO. 1623 is depicted, mitochondric is mentioned; which is operable linked, too.

[102484] In another embodiment thereof the expression cassette does not comprise an additional transit peptide in case in Table I, application no. 20, in column 6 in the same line as SEQ ID NO. 1623 is depicted, non-targeted is mentioned.

[102485] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 109717, or a homolog or fragment thereof, or a homolog thereof being depicted in Table I, application no. 20, column 8, in the same line as SEQ ID NO. 109717, preferably the coding region thereof, a homolog or a fragment thereof; which are operable linked.

[102486] In an embodiment thereof the expression cassette comprises in addition c) a nucleic acid molecule encoding a transit peptide, especially a plastidal transit petide in case in Table I, application no. 20, in column 6 in the same line as SEQ ID NO. 109717 is depicted, plastidic is mentioned, or a mitochondrial transit peptide in case in Table I, application no. 20, in column 6 in the same line as SEQ ID NO. 109717 is depicted, mitochondric is mentioned; which is operable linked, too.

[102487] In another embodiment thereof the expression cassette does not comprise an additional transit peptide in case in Table I, application no. 20, in column 6 in the same line as SEQ ID NO. 109717 is depicted, non-targeted is mentioned.

[102488] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 142929, or a homolog or fragment thereof, or a homolog thereof being depicted in Table I, application no. 20, column 8, in the same line as SEQ ID NO. 142929, preferably the coding region thereof, a homolog or a fragment thereof; which are operable linked.

[102489] In an embodiment thereof the expression cassette comprises in addition c) a nucleic acid molecule encoding a transit peptide, especially a plastidal transit petide in case in Table I, application no. 20, in column 6 in the same line as SEQ ID NO. 142929 is depicted, plastidic is mentioned, or a mitochondrial transit peptide in case in Table I, application no. 20, in column 6 in the same line as SEQ ID NO. 142929 is depicted, mitochondric is mentioned; which is operable linked, too.

[102490] In another embodiment thereof the expression cassette does not comprise an additional transit peptide in case in Table I, application no. 20, in column 6 in the same line as SEQ ID NO. 142929 is depicted, non-targeted is mentioned.

[102491] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 70006, or a homolog or fragment thereof, or a homolog thereof being depicted in Table I, application no. 20, column 8, in the same line as SEQ ID NO. 70006, preferably the coding region thereof, a homolog or a fragment thereof; which are operable linked.

[102492] In an embodiment thereof the expression cassette comprises in addition c) a nucleic acid molecule encoding a transit peptide, especially a plastidal transit petide in case in Table I, application no. 20, in column 6 in the same line as SEQ ID NO. 70006 is depicted, plastidic is mentioned, or a mitochondrial transit peptide in case in Table I, application no. 20, in column 6 in the same line as SEQ ID NO. 70006 is depicted, mitochondric is mentioned; which is operable linked, too.

[102493] In another embodiment thereof the expression cassette does not comprise an additional transit peptide in case in Table I, application no. 20, in column 6 in the same line as SEQ ID NO. 70006 is depicted, non-targeted is mentioned.

[102494] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 29286, or a homolog or fragment thereof, or a homolog thereof being depicted in Table I, application no. 20, column 8, in the same line as SEQ ID NO. 29286, preferably the coding region thereof, a homolog or a fragment thereof; which are operable linked.

[102495] In an embodiment thereof the expression cassette comprises in addition c) a nucleic acid molecule encoding a transit peptide, especially a plastidal transit petide in case in Table I, application no. 20, in column 6 in the same line as SEQ ID NO. 29286 is depicted, plastidic is mentioned, or a mitochondrial transit peptide in case in Table I, application no. 20, in column 6 in the same line as SEQ ID NO. 29286 is depicted, mitochondric is mentioned; which is operable linked, too.

[102496] In another embodiment thereof the expression cassette does not comprise an additional transit peptide in case in Table I, application no. 20, in column 6 in the same line as SEQ ID NO. 29286 is depicted, non-targeted is mentioned.

[102497] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 32308, or a homolog or fragment thereof, or a homolog thereof being depicted in Table I, application no. 20, column 8, in the same line as SEQ ID NO. 32308, preferably the coding region thereof, a homolog or a fragment thereof; which are operable linked.

[102498] In an embodiment thereof the expression cassette comprises in addition c) a nucleic acid molecule encoding a transit peptide, especially a plastidal transit petide in case in Table I, application no. 20, in column 6 in the same line as SEQ ID NO. 32308 is depicted, plastidic is mentioned, or a mitochondrial transit peptide in case in Table I, application no. 20, in column 6 in the same line as SEQ ID NO. 32308 is depicted, mitochondric is mentioned; which is operable linked, too.

[102499] In another embodiment thereof the expression cassette does not comprise an additional transit peptide in case in Table I, application no. 20, in column 6 in the same line as SEQ ID NO. 32308 is depicted, non-targeted is mentioned.

[102500] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 154509, or a homolog or fragment thereof, or a homolog thereof being depicted in Table I, application no. 20, column 8, in the same line as SEQ ID NO. 154509, preferably the coding region thereof, a homolog or a fragment thereof; which are operable linked.

[102501] In an embodiment thereof the expression cassette comprises in addition c) a nucleic acid molecule encoding a transit peptide, especially a plastidal transit petide in case in Table I, application no. 20, in column 6 in the same line as SEQ ID NO. 154509 is depicted, plastidic is mentioned, or a mitochondrial transit peptide in case in Table I, application no. 20, in column 6 in the same line as SEQ ID NO. 154509 is depicted, mitochondric is mentioned; which is operable linked, too.

[102502] In another embodiment thereof the expression cassette does not comprise an additional transit peptide in case in Table I, application no. 20, in column 6 in the same line as SEQ ID NO. 154509 is depicted, non-targeted is mentioned.

[102503] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 7081, or a homolog or fragment thereof, or a homolog thereof being depicted in Table I, application no. 20, column 8, in the same line as SEQ ID NO. 7081, preferably the coding region thereof, a homolog or a fragment thereof; which are operable linked.

[102504] In an embodiment thereof the expression cassette comprises in addition c) a nucleic acid molecule encoding a transit peptide, especially a plastidal transit petide in case in Table I, application no. 20, in column 6 in the same line as SEQ ID NO. 7081 is depicted, plastidic is mentioned, or a mitochondrial transit peptide in case in Table I, application no. 20, in column 6 in the same line as SEQ ID NO. 7081 is depicted, mitochondric is mentioned; which is operable linked, too.

[102505] In another embodiment thereof the expression cassette does not comprise an additional transit peptide in case in Table I, application no. 20, in column 6 in the same line as SEQ ID NO. 7081 is depicted, non-targeted is mentioned.

[102506] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 155230, or a homolog or fragment thereof, or a homolog thereof being depicted in Table I, application no. 20, column 8, in the same line as SEQ ID NO. 155230, preferably the coding region thereof, a homolog or a fragment thereof; which are operable linked.

[102507] In an embodiment thereof the expression cassette comprises in addition c) a nucleic acid molecule encoding a transit peptide, especially a plastidal transit petide in case in Table I, application no. 20, in column 6 in the same line as SEQ ID NO. 155230 is depicted, plastidic is mentioned, or a mitochondrial transit peptide in case in Table I, application no. 20, in column 6 in the same line as SEQ ID NO. 155230 is depicted, mitochondric is mentioned; which is operable linked, too.

[102508] In another embodiment thereof the expression cassette does not comprise an additional transit peptide in case in Table I, application no. 20, in column 6 in the same line as SEQ ID NO. 155230 is depicted, non-targeted is mentioned.

[102509] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 155370, or a homolog or fragment thereof, or a homolog thereof being depicted in Table I, application no. 20, column 8, in the same line as SEQ ID NO. 155370, preferably the coding region thereof, a homolog or a fragment thereof; which are operable linked.

[102510] In an embodiment thereof the expression cassette comprises in addition c) a nucleic acid molecule encoding a transit peptide, especially a plastidal transit petide in case in Table I, application no. 20, in column 6 in the same line as SEQ ID NO. 155370 is depicted, plastidic is mentioned, or a mitochondrial transit peptide in case in Table I, application no. 20, in column 6 in the same line as SEQ ID NO. 155370 is depicted, mitochondric is mentioned; which is operable linked, too.

[102511] In another embodiment thereof the expression cassette does not comprise an additional transit peptide in case in Table I, application no. 20, in column 6 in the same line as SEQ ID NO. 155370 is depicted, non-targeted is mentioned.

[102512] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 143954, or a homolog or fragment thereof, or a homolog thereof being depicted in Table I, application no. 20, column 8, in the same line as SEQ ID NO. 143954, preferably the coding region thereof, a homolog or a fragment thereof; which are operable linked.

[102513] In an embodiment thereof the expression cassette comprises in addition c) a nucleic acid molecule encoding a transit peptide, especially a plastidal transit petide in case in Table I, application no. 20, in column 6 in the same line as SEQ ID NO. 143954 is depicted, plastidic is mentioned, or a mitochondrial transit peptide in case in Table I, application no. 20, in column 6 in the same line as SEQ ID NO. 143954 is depicted, mitochondric is mentioned; which is operable linked, too.

[102514] In another embodiment thereof the expression cassette does not comprise an additional transit peptide in case in Table I, application no. 20, in column 6 in the same line as SEQ ID NO. 143954 is depicted, non-targeted is mentioned.

[102515] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 155613, or a homolog or fragment thereof, or a homolog thereof being depicted in Table I, application no. 20, column 8, in the same line as SEQ ID NO. 155613, preferably the coding region thereof, a homolog or a fragment thereof; which are operable linked.

[102516] In an embodiment thereof the expression cassette comprises in addition c) a nucleic acid molecule encoding a transit peptide, especially a plastidal transit petide in case in Table I, application no. 20, in column 6 in the same line as SEQ ID NO. 155613 is depicted, plastidic is mentioned, or a mitochondrial transit peptide in case in Table I, application no. 20, in column 6 in the same line as SEQ ID NO. 155613 is depicted, mitochondric is mentioned; which is operable linked, too.

[102517] In another embodiment thereof the expression cassette does not comprise an additional transit peptide in case in Table I, application no. 20, in column 6 in the same line as SEQ ID NO. 155613 is depicted, non-targeted is mentioned.

[102518] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 155657, or a homolog or fragment thereof, or a homolog thereof being depicted in Table I, application no. 20, column 8, in the same line as SEQ ID NO. 155657, preferably the coding region thereof, a homolog or a fragment thereof; which are operable linked.

[102519] In an embodiment thereof the expression cassette comprises in addition c) a nucleic acid molecule encoding a transit peptide, especially a plastidal transit petide in case in Table I, application no. 20, in column 6 in the same line as SEQ ID NO. 155657 is depicted, plastidic is mentioned, or a mitochondrial transit peptide in case in Table I, application no. 20, in column 6 in the same line as SEQ ID NO. 155657 is depicted, mitochondric is mentioned; which is operable linked, too.

[102520] In another embodiment thereof the expression cassette does not comprise an additional transit peptide in case in Table I, application no. 20, in column 6 in the same line as SEQ ID NO. 155657 is depicted, non-targeted is mentioned.

[102521] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 155665, or a homolog or fragment thereof, or a homolog thereof being depicted in Table I, application no. 20, column 8, in the same line as SEQ ID NO. 155665, preferably the coding region thereof, a homolog or a fragment thereof; which are operable linked.

[102522] In an embodiment thereof the expression cassette comprises in addition c) a nucleic acid molecule encoding a transit peptide, especially a plastidal transit petide in case in Table I, application no. 20, in column 6 in the same line as SEQ ID NO. 155665 is depicted, plastidic is mentioned, or a mitochondrial transit peptide in case in Table I, application no. 20, in column 6 in the same line as SEQ ID NO. 155665 is depicted, mitochondric is mentioned; which is operable linked, too.

[102523] In another embodiment thereof the expression cassette does not comprise an additional transit peptide in case in Table I, application no. 20, in column 6 in the same line as SEQ ID NO. 155665 is depicted, non-targeted is mentioned.

[102524] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 37503, or a homolog or fragment thereof, or a homolog thereof being depicted in Table I, application no. 20, column 8, in the same line as SEQ ID NO. 37503, preferably the coding region thereof, a homolog or a fragment thereof; which are operable linked.

[102525] In an embodiment thereof the expression cassette comprises in addition c) a nucleic acid molecule encoding a transit peptide, especially a plastidal transit petide in case in Table I, application no. 20, in column 6 in the same line as SEQ ID NO. 37503 is depicted, plastidic is mentioned, or a mitochondrial transit peptide in case in Table I, application no. 20, in column 6 in the same line as SEQ ID NO. 37503 is depicted, mitochondric is mentioned; which is operable linked, too.

[102526] In another embodiment thereof the expression cassette does not comprise an additional transit peptide in case in Table I, application no. 20, in column 6 in the same line as SEQ ID NO. 37503 is depicted, non-targeted is mentioned.

[102527] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 155890, or a homolog or fragment thereof, or a homolog thereof being depicted in Table I, application no. 20, column 8, in the same line as SEQ ID NO. 155890, preferably the coding region thereof, a homolog or a fragment thereof; which are operable linked.

[102528] In an embodiment thereof the expression cassette comprises in addition c) a nucleic acid molecule encoding a transit peptide, especially a plastidal transit petide in case in Table I, application no. 20, in column 6 in the same line as SEQ ID NO. 155890 is depicted, plastidic is mentioned, or a mitochondrial transit peptide in case in Table I, application no. 20, in column 6 in the same line as SEQ ID NO. 155890 is depicted, mitochondric is mentioned; which is operable linked, too.

[102529] In another embodiment thereof the expression cassette does not comprise an additional transit peptide in case in Table I, application no. 20, in column 6 in the same line as SEQ ID NO. 155890 is depicted, non-targeted is mentioned.

[102530] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 76890, or a homolog or fragment thereof, or a homolog thereof being depicted in Table I, application no. 20, column 8, in the same line as SEQ ID NO. 76890, preferably the coding region thereof, a homolog or a fragment thereof; which are operable linked.

[102531] In an embodiment thereof the expression cassette comprises in addition c) a nucleic acid molecule encoding a transit peptide, especially a plastidal transit petide in case in Table I, application no. 20, in column 6 in the same line as SEQ ID NO. 76890 is depicted, plastidic is mentioned, or a mitochondrial transit peptide in case in Table I, application no. 20, in column 6 in the same line as SEQ ID NO. 76890 is depicted, mitochondric is mentioned; which is operable linked, too.

[102532] In another embodiment thereof the expression cassette does not comprise an additional transit peptide in case in Table I, application no. 20, in column 6 in the same line as SEQ ID NO. 76890 is depicted, non-targeted is mentioned.

[102533] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38289, or a homolog or fragment thereof, or a homolog thereof being depicted in Table I, application no. 20, column 8, in the same line as SEQ ID NO. 38289, preferably the coding region thereof, a homolog or a fragment thereof; which are operable linked.

[102534] In an embodiment thereof the expression cassette comprises in addition c) a nucleic acid molecule encoding a transit peptide, especially a plastidal transit petide in case in Table I, application no. 20, in column 6 in the same line as SEQ ID NO. 38289 is depicted, plastidic is mentioned, or a mitochondrial transit peptide in case in Table I, application no. 20, in column 6 in the same line as SEQ ID NO. 38289 is depicted, mitochondric is mentioned; which is operable linked, too.

[102535] In another embodiment thereof the expression cassette does not comprise an additional transit peptide in case in Table I, application no. 20, in column 6 in the same line as SEQ ID NO. 38289 is depicted, non-targeted is mentioned.

[102536] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38300, or a homolog or fragment thereof, or a homolog thereof being depicted in Table I, application no. 20, column 8, in the same line as SEQ ID NO. 38300, preferably the coding region thereof, a homolog or a fragment thereof; which are operable linked.

[102537] In an embodiment thereof the expression cassette comprises in addition c) a nucleic acid molecule encoding a transit peptide, especially a plastidal transit petide in case in Table I, application no. 20, in column 6 in the same line as SEQ ID NO. 38300 is depicted, plastidic is mentioned, or a mitochondrial transit peptide in case in Table I, application no. 20, in column 6 in the same line as SEQ ID NO. 38300 is depicted, mitochondric is mentioned; which is operable linked, too.

[102538] In another embodiment thereof the expression cassette does not comprise an additional transit peptide in case in Table I, application no. 20, in column 6 in the same line as SEQ ID NO. 38300 is depicted, non-targeted is mentioned.

[102539] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 38345, or a homolog or fragment thereof, or a homolog thereof being depicted in Table I, application no. 20, column 8, in the same line as SEQ ID NO. 38345, preferably the coding region thereof, a homolog or a fragment thereof; which are operable linked.

[102540] In an embodiment thereof the expression cassette comprises in addition c) a nucleic acid molecule encoding a transit peptide, especially a plastidal transit petide in case in Table I, application no. 20, in column 6 in the same line as SEQ ID NO. 38345 is depicted, plastidic is mentioned, or a mitochondrial transit peptide in case in Table I, application no. 20, in column 6 in the same line as SEQ ID NO. 38345 is depicted, mitochondric is mentioned; which is operable linked, too.

[102541] In another embodiment thereof the expression cassette does not comprise an additional transit peptide in case in Table I, application no. 20, in column 6 in the same line as SEQ ID NO. 38345 is depicted, non-targeted is mentioned.

[102542] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 156392, or a homolog or fragment thereof, or a homolog thereof being depicted in Table I, application no. 20, column 8, in the same line as SEQ ID NO. 156392, preferably the coding region thereof, a homolog or a fragment thereof; which are operable linked.

[102543] In an embodiment thereof the expression cassette comprises in addition c) a nucleic acid molecule encoding a transit peptide, especially a plastidal transit petide in case in Table I, application no. 20, in column 6 in the same line as SEQ ID NO. 156392 is depicted, plastidic is mentioned, or a mitochondrial transit peptide in case in Table I, application no. 20, in column 6 in the same line as SEQ ID NO. 156392 is depicted, mitochondric is mentioned; which is operable linked, too.

[102544] In another embodiment thereof the expression cassette does not comprise an additional transit peptide in case in Table I, application no. 20, in column 6 in the same line as SEQ ID NO. 156392 is depicted, non-targeted is mentioned.

[102545] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 78753, or a homolog or fragment thereof, or a homolog thereof being depicted in Table I, application no. 20, column 8, in the same line as SEQ ID NO. 78753, preferably the coding region thereof, a homolog or a fragment thereof; which are operable linked.

[102546] In an embodiment thereof the expression cassette comprises in addition c) a nucleic acid molecule encoding a transit peptide, especially a plastidal transit petide in case in Table I, application no. 20, in column 6 in the same line as SEQ ID NO. 78753 is depicted, plastidic is mentioned, or a mitochondrial transit peptide in case in Table I, application no. 20, in column 6 in the same line as SEQ ID NO. 78753 is depicted, mitochondric is mentioned; which is operable linked, too.

[102547] In another embodiment thereof the expression cassette does not comprise an additional transit peptide in case in Table I, application no. 20, in column 6 in the same line as SEQ ID NO. 78753 is depicted, non-targeted is mentioned.

[102548] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 156542, or a homolog or fragment thereof, or a homolog thereof being depicted in Table I, application no. 20, column 8, in the same line as SEQ ID NO. 156542, preferably the coding region thereof, a homolog or a fragment thereof; which are operable linked.

[102549] In an embodiment thereof the expression cassette comprises in addition c) a nucleic acid molecule encoding a transit peptide, especially a plastidal transit petide in case in Table I, application no. 20, in column 6 in the same line as SEQ ID NO. 156542 is depicted, plastidic is mentioned, or a mitochondrial transit peptide in case in Table I, application no. 20, in column 6 in the same line as SEQ ID NO. 156542 is depicted, mitochondric is mentioned; which is operable linked, too.

[102550] In another embodiment thereof the expression cassette does not comprise an additional transit peptide in case in Table I, application no. 20, in column 6 in the same line as SEQ ID NO. 156542 is depicted, non-targeted is mentioned.

[102551] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 79181, or a homolog or fragment thereof, or a homolog thereof being depicted in Table I, application no. 20, column 8, in the same line as SEQ ID NO. 79181, preferably the coding region thereof, a homolog or a fragment thereof; which are operable linked.

[102552] In an embodiment thereof the expression cassette comprises in addition c) a nucleic acid molecule encoding a transit peptide, especially a plastidal transit petide in case in Table I, application no. 20, in column 6 in the same line as SEQ ID NO. 79181 is depicted, plastidic is mentioned, or a mitochondrial transit peptide in case in Table I, application no. 20, in column 6 in the same line as SEQ ID NO. 79181 is depicted, mitochondric is mentioned; which is operable linked, too.

[102553] In another embodiment thereof the expression cassette does not comprise an additional transit peptide in case in Table I, application no. 20, in column 6 in the same line as SEQ ID NO. 79181 is depicted, non-targeted is mentioned.

[102554] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 79217, or a homolog or fragment thereof, or a homolog thereof being depicted in Table I, application no. 20, column 8, in the same line as SEQ ID NO. 79217, preferably the coding region thereof, a homolog or a fragment thereof; which are operable linked.

[102555] In an embodiment thereof the expression cassette comprises in addition c) a nucleic acid molecule encoding a transit peptide, especially a plastidal transit petide in case in Table I, application no. 20, in column 6 in the same line as SEQ ID NO. 79217 is depicted, plastidic is mentioned, or a mitochondrial transit peptide in case in Table I, application no. 20, in column 6 in the same line as SEQ ID NO. 79217 is depicted, mitochondric is mentioned; which is operable linked, too.

[102556] In another embodiment thereof the expression cassette does not comprise an additional transit peptide in case in Table I, application no. 20, in column 6 in the same line as SEQ ID NO. 79217 is depicted, non-targeted is mentioned.

[102557] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 104277, or a homolog or fragment thereof, or a homolog thereof being depicted in Table I, application no. 20, column 8, in the same line as SEQ ID NO. 104277, preferably the coding region thereof, a homolog or a fragment thereof; which are operable linked.

[102558] In an embodiment thereof the expression cassette comprises in addition c) a nucleic acid molecule encoding a transit peptide, especially a plastidal transit petide in case in Table I, application no. 20, in column 6 in the same line as SEQ ID NO. 104277 is depicted, plastidic is mentioned, or a mitochondrial transit peptide in case in Table I, application no. 20, in column 6 in the same line as SEQ ID NO. 104277 is depicted, mitochondric is mentioned; which is operable linked, too.

[102559] In another embodiment thereof the expression cassette does not comprise an additional transit peptide in case in Table I, application no. 20, in column 6 in the same line as SEQ ID NO. 104277 is depicted, non-targeted is mentioned.

[102560] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 156810, or a homolog or fragment thereof, or a homolog thereof being depicted in Table I, application no. 20, column 8, in the same line as SEQ ID NO. 156810, preferably the coding region thereof, a homolog or a fragment thereof; which are operable linked.

[102561] In an embodiment thereof the expression cassette comprises in addition c) a nucleic acid molecule encoding a transit peptide, especially a plastidal transit petide in case in Table I, application no. 20, in column 6 in the same line as SEQ ID NO. 156810 is depicted, plastidic is mentioned, or a mitochondrial transit peptide in case in Table I, application no. 20, in column 6 in the same line as SEQ ID NO. 156810 is depicted, mitochondric is mentioned; which is operable linked, too.

[102562] In another embodiment thereof the expression cassette does not comprise an additional transit peptide in case in Table I, application no. 20, in column 6 in the same line as SEQ ID NO. 156810 is depicted, non-targeted is mentioned.

[102563] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 146238, or a homolog or fragment thereof, or a homolog thereof being depicted in Table I, application no. 20, column 8, in the same line as SEQ ID NO. 146238, preferably the coding region thereof, a homolog or a fragment thereof; which are operable linked.

[102564] In an embodiment thereof the expression cassette comprises in addition c) a nucleic acid molecule encoding a transit peptide, especially a plastidal transit petide in case in Table I, application no. 20, in column 6 in the same line as SEQ ID NO. 146238 is depicted, plastidic is mentioned, or a mitochondrial transit peptide in case in Table I, application no. 20, in column 6 in the same line as SEQ ID NO. 146238 is depicted, mitochondric is mentioned; which is operable linked, too.

[102565] In another embodiment thereof the expression cassette does not comprise an additional transit peptide in case in Table I, application no. 20, in column 6 in the same line as SEQ ID NO. 146238 is depicted, non-targeted is mentioned.

[102566] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39237, or a homolog or fragment thereof, or a homolog thereof being depicted in Table I, application no. 20, column 8, in the same line as SEQ ID NO. 39237, preferably the coding region thereof, a homolog or a fragment thereof; which are operable linked.

[102567] In an embodiment thereof the expression cassette comprises in addition c) a nucleic acid molecule encoding a transit peptide, especially a plastidal transit petide in case in Table I, application no. 20, in column 6 in the same line as SEQ ID NO. 39237 is depicted, plastidic is mentioned, or a mitochondrial transit peptide in case in Table I, application no. 20, in column 6 in the same line as SEQ ID NO. 39237 is depicted, mitochondric is mentioned; which is operable linked, too.

[102568] In another embodiment thereof the expression cassette does not comprise an additional transit peptide in case in Table I, application no. 20, in column 6 in the same line as SEQ ID NO. 39237 is depicted, non-targeted is mentioned.

[102569] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 39300, or a homolog or fragment thereof, or a homolog thereof being depicted in Table I, application no. 20, column 8, in the same line as SEQ ID NO. 39300, preferably the coding region thereof, a homolog or a fragment thereof; which are operable linked.

[102570] In an embodiment thereof the expression cassette comprises in addition c) a nucleic acid molecule encoding a transit peptide, especially a plastidal transit petide in case in Table I, application no. 20, in column 6 in the same line as SEQ ID NO. 39300 is depicted, plastidic is mentioned, or a mitochondrial transit peptide in case in Table I, application no. 20, in column 6 in the same line as SEQ ID NO. 39300 is depicted, mitochondric is mentioned; which is operable linked, too.

[102571] In another embodiment thereof the expression cassette does not comprise an additional transit peptide in case in Table I, application no. 20, in column 6 in the same line as SEQ ID NO. 39300 is depicted, non-targeted is mentioned.

[102572] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 115628, or a homolog or fragment thereof, or a homolog thereof being depicted in Table I, application no. 20, column 8, in the same line as SEQ ID NO. 115628, preferably the coding region thereof, a homolog or a fragment thereof; which are operable linked.

[102573] In an embodiment thereof the expression cassette comprises in addition c) a nucleic acid molecule encoding a transit peptide, especially a plastidal transit petide in case in Table I, application no. 20, in column 6 in the same line as SEQ ID NO. 115628 is depicted, plastidic is mentioned, or a mitochondrial transit peptide in case in Table I, application no. 20, in column 6 in the same line as SEQ ID NO. 115628 is depicted, mitochondric is mentioned; which is operable linked, too.

[102574] In another embodiment thereof the expression cassette does not comprise an additional transit peptide in case in Table I, application no. 20, in column 6 in the same line as SEQ ID NO. 115628 is depicted, non-targeted is mentioned.

[102575] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 156910, or a homolog or fragment thereof, or a homolog thereof being depicted in Table I, application no. 20, column 8, in the same line as SEQ ID NO. 156910, preferably the coding region thereof, a homolog or a fragment thereof; which are operable linked.

[102576] In an embodiment thereof the expression cassette comprises in addition c) a nucleic acid molecule encoding a transit peptide, especially a plastidal transit petide in case in Table I, application no. 20, in column 6 in the same line as SEQ ID NO. 156910 is depicted, plastidic is mentioned, or a mitochondrial transit peptide in case in Table I, application no. 20, in column 6 in the same line as SEQ ID NO. 156910 is depicted, mitochondric is mentioned; which is operable linked, too.

[102577] In another embodiment thereof the expression cassette does not comprise an additional transit peptide in case in Table I, application no. 20, in column 6 in the same line as SEQ ID NO. 156910 is depicted, non-targeted is mentioned.

[102578] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 146288, or a homolog or fragment thereof, or a homolog thereof being depicted in Table I, application no. 20, column 8, in the same line as SEQ ID NO. 146288, preferably the coding region thereof, a homolog or a fragment thereof; which are operable linked.

[102579] In an embodiment thereof the expression cassette comprises in addition c) a nucleic acid molecule encoding a transit peptide, especially a plastidal transit petide in case in Table I, application no. 20, in column 6 in the same line as SEQ ID NO. 146288 is depicted, plastidic is mentioned, or a mitochondrial transit peptide in case in Table I, application no. 20, in column 6 in the same line as SEQ ID NO. 146288 is depicted, mitochondric is mentioned; which is operable linked, too.

[102580] In another embodiment thereof the expression cassette does not comprise an additional transit peptide in case in Table I, application no. 20, in column 6 in the same line as SEQ ID NO. 146288 is depicted, non-targeted is mentioned.

[102581] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 81451, or a homolog or fragment thereof, or a homolog thereof being depicted in Table I, application no. 20, column 8, in the same line as SEQ ID NO. 81451, preferably the coding region thereof, a homolog or a fragment thereof; which are operable linked.

[102582] In an embodiment thereof the expression cassette comprises in addition c) a nucleic acid molecule encoding a transit peptide, especially a plastidal transit petide in case in Table I, application no. 20, in column 6 in the same line as SEQ ID NO. 81451 is depicted, plastidic is mentioned, or a mitochondrial transit peptide in case in Table I, application no. 20, in column 6 in the same line as SEQ ID NO. 81451 is depicted, mitochondric is mentioned; which is operable linked, too.

[102583] In another embodiment thereof the expression cassette does not comprise an additional transit peptide in case in Table I, application no. 20, in column 6 in the same line as SEQ ID NO. 81451 is depicted, non-targeted is mentioned.

[102584] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 81576, or a homolog or fragment thereof, or a homolog thereof being depicted in Table I, application no. 20, column 8, in the same line as SEQ ID NO. 81576, preferably the coding region thereof, a homolog or a fragment thereof; which are operable linked.

[102585] In an embodiment thereof the expression cassette comprises in addition c) a nucleic acid molecule encoding a transit peptide, especially a plastidal transit petide in case in Table I, application no. 20, in column 6 in the same line as SEQ ID NO. 81576 is depicted, plastidic is mentioned, or a mitochondrial transit peptide in case in Table I, application no. 20, in column 6 in the same line as SEQ ID NO. 81576 is depicted, mitochondric is mentioned; which is operable linked, too.

[102586] In another embodiment thereof the expression cassette does not comprise an additional transit peptide in case in Table I, application no. 20, in column 6 in the same line as SEQ ID NO. 81576 is depicted, non-targeted is mentioned.

[102587] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 157128, or a homolog or fragment thereof, or a homolog thereof being depicted in Table I, application no. 20, column 8, in the same line as SEQ ID NO. 157128, preferably the coding region thereof, a homolog or a fragment thereof; which are operable linked.

[102588] In an embodiment thereof the expression cassette comprises in addition c) a nucleic acid molecule encoding a transit peptide, especially a plastidal transit petide in case in Table I, application no. 20, in column 6 in the same line as SEQ ID NO. 157128 is depicted, plastidic is mentioned, or a mitochondrial transit peptide in case in Table I, application no. 20, in column 6 in the same line as SEQ ID NO. 157128 is depicted, mitochondric is mentioned; which is operable linked, too.

[102589] In another embodiment thereof the expression cassette does not comprise an additional transit peptide in case in Table I, application no. 20, in column 6 in the same line as SEQ ID NO. 157128 is depicted, non-targeted is mentioned.

[102590] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 41499, or a homolog or fragment thereof, or a homolog thereof being depicted in Table I, application no. 20, column 8, in the same line as SEQ ID NO. 41499, preferably the coding region thereof, a homolog or a fragment thereof; which are operable linked.

[102591] In an embodiment thereof the expression cassette comprises in addition c) a nucleic acid molecule encoding a transit peptide, especially a plastidal transit petide in case in Table I, application no. 20, in column 6 in the same line as SEQ ID NO. 41499 is depicted, plastidic is mentioned, or a mitochondrial transit peptide in case in Table I, application no. 20, in column 6 in the same line as SEQ ID NO. 41499 is depicted, mitochondric is mentioned; which is operable linked, too.

[102592] In another embodiment thereof the expression cassette does not comprise an additional transit peptide in case in Table I, application no. 20, in column 6 in the same line as SEQ ID NO. 41499 is depicted, non-targeted is mentioned.

[102593] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 157321, or a homolog or fragment thereof, or a homolog thereof being depicted in Table I, application no. 20, column 8, in the same line as SEQ ID NO. 157321, preferably the coding region thereof, a homolog or a fragment thereof; which are operable linked.

[102594] In an embodiment thereof the expression cassette comprises in addition c) a nucleic acid molecule encoding a transit peptide, especially a plastidal transit petide in case in Table I, application no. 20, in column 6 in the same line as SEQ ID NO. 157321 is depicted, plastidic is mentioned, or a mitochondrial transit peptide in case in Table I, application no. 20, in column 6 in the same line as SEQ ID NO. 157321 is depicted, mitochondric is mentioned; which is operable linked, too.

[102595] In another embodiment thereof the expression cassette does not comprise an additional transit peptide in case in Table I, application no. 20, in column 6 in the same line as SEQ ID NO. 157321 is depicted, non-targeted is mentioned.

[102596] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 157450, or a homolog or fragment thereof, or a homolog thereof being depicted in Table I, application no. 20, column 8, in the same line as SEQ ID NO. 157450, preferably the coding region thereof, a homolog or a fragment thereof; which are operable linked.

[102597] In an embodiment thereof the expression cassette comprises in addition c) a nucleic acid molecule encoding a transit peptide, especially a plastidal transit petide in case in Table I, application no. 20, in column 6 in the same line as SEQ ID NO. 157450 is depicted, plastidic is mentioned, or a mitochondrial transit peptide in case in Table I, application no. 20, in column 6 in the same line as SEQ ID NO. 157450 is depicted, mitochondric is mentioned; which is operable linked, too.

[102598] In another embodiment thereof the expression cassette does not comprise an additional transit peptide in case in Table I, application no. 20, in column 6 in the same line as SEQ ID NO. 157450 is depicted, non-targeted is mentioned.

[102599] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 125264, or a homolog or fragment thereof, or a homolog thereof being depicted in Table I, application no. 20, column 8, in the same line as SEQ ID NO. 125264, preferably the coding region thereof, a homolog or a fragment thereof; which are operable linked.

[102600] In an embodiment thereof the expression cassette comprises in addition c) a nucleic acid molecule encoding a transit peptide, especially a plastidal transit petide in case in Table I, application no. 20, in column 6 in the same line as SEQ ID NO. 125264 is depicted, plastidic is mentioned, or a mitochondrial transit peptide in case in Table I, application no. 20, in column 6 in the same line as SEQ ID NO. 125264 is depicted, mitochondric is mentioned; which is operable linked, too.

[102601] In another embodiment thereof the expression cassette does not comprise an additional transit peptide in case in Table I, application no. 20, in column 6 in the same line as SEQ ID NO. 125264 is depicted, non-targeted is mentioned.

[102602] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 46850, or a homolog or fragment thereof, or a homolog thereof being depicted in Table I, application no. 20, column 8, in the same line as SEQ ID NO. 46850, preferably the coding region thereof, a homolog or a fragment thereof; which are operable linked.

[102603] In an embodiment thereof the expression cassette comprises in addition c) a nucleic acid molecule encoding a transit peptide, especially a plastidal transit petide in case in Table I, application no. 20, in column 6 in the same line as SEQ ID NO. 46850 is depicted, plastidic is mentioned, or a mitochondrial transit peptide in case in Table I, application no. 20, in column 6 in the same line as SEQ ID NO. 46850 is depicted, mitochondric is mentioned; which is operable linked, too.

[102604] In another embodiment thereof the expression cassette does not comprise an additional transit peptide in case in Table I, application no. 20, in column 6 in the same line as SEQ ID NO. 46850 is depicted, non-targeted is mentioned.

[102605] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 59370, or a homolog or fragment thereof, or a homolog thereof being depicted in Table I, application no. 20, column 8, in the same line as SEQ ID NO. 59370, preferably the coding region thereof, a homolog or a fragment thereof; which are operable linked.

[102606] In an embodiment thereof the expression cassette comprises in addition c) a nucleic acid molecule encoding a transit peptide, especially a plastidal transit petide in case in Table I, application no. 20, in column 6 in the same line as SEQ ID NO. 59370 is depicted, plastidic is mentioned, or a mitochondrial transit peptide in case in Table I, application no. 20, in column 6 in the same line as SEQ ID NO. 59370 is depicted, mitochondric is mentioned; which is operable linked, too.

[102607] In another embodiment thereof the expression cassette does not comprise an additional transit peptide in case in Table I, application no. 20, in column 6 in the same line as SEQ ID NO. 59370 is depicted, non-targeted is mentioned.

[102608] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 60301, or a homolog or fragment thereof, or a homolog thereof being depicted in Table I, application no. 20, column 8, in the same line as SEQ ID NO. 60301, preferably the coding region thereof, a homolog or a fragment thereof; which are operable linked.

[102609] In an embodiment thereof the expression cassette comprises in addition c) a nucleic acid molecule encoding a transit peptide, especially a plastidal transit petide in case in Table I, application no. 20, in column 6 in the same line as SEQ ID NO. 60301 is depicted, plastidic is mentioned, or a mitochondrial transit peptide in case in Table I, application no. 20, in column 6 in the same line as SEQ ID NO. 60301 is depicted, mitochondric is mentioned; which is operable linked, too.

[102610] In another embodiment thereof the expression cassette does not comprise an additional transit peptide in case in Table I, application no. 20, in column 6 in the same line as SEQ ID NO. 60301 is depicted, non-targeted is mentioned.

[102611] In an embodiment the present invention relates to an expresssion cassette comprising a) a promoter, preferably a promoter selected from the group consisting of Big35S, PCUbi, Super and USP, in particular p-PcUBI; and b) the nucleic acid SEQ ID NO. 61553, or a homolog or fragment thereof, or a homolog thereof being depicted in Table I, application no. 20, column 8, in the same line as SEQ ID NO. 61553, preferably the coding region thereof, a homolog or a fragment thereof; which are operable linked.

[102612] In an embodiment thereof the expression cassette comprises in addition c) a nucleic acid molecule encoding a transit peptide, especially a plastidal transit petide in case in Table I, application no. 20, in column 6 in the same line as SEQ ID NO. 61553 is depicted, plastidic is mentioned, or a mitochondrial transit peptide in case in Table I, application no. 20, in column 6 in the same line as SEQ ID NO. 61553 is depicted, mitochondric is mentioned; which is operable linked, too.

[102613] In another embodiment thereof the expression cassette does not comprise an additional transit peptide in case in Table I, application no. 20, in column 6 in the same line as SEQ ID NO. 61553 is depicted, non-targeted is mentioned.

[102614] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 88879, or a homolog or fragment thereof, or a homolog thereof being depicted in Table I, application no. 20, column 8, in the same line as SEQ ID NO. 88879, preferably the coding region thereof, a homolog or a fragment thereof; which are operable linked.

[102615] In an embodiment thereof the expression cassette comprises in addition c) a nucleic acid molecule encoding a transit peptide, especially a plastidal transit petide in case in Table I, application no. 20, in column 6 in the same line as SEQ ID NO. 88879 is depicted, plastidic is mentioned, or a mitochondrial transit peptide in case in Table I, application no. 20, in column 6 in the same line as SEQ ID NO. 88879 is depicted, mitochondric is mentioned; which is operable linked, too.

[102616] In another embodiment thereof the expression cassette does not comprise an additional transit peptide in case in Table I, application no. 20, in column 6 in the same line as SEQ ID NO. 88879 is depicted, non-targeted is mentioned.

[102617] In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 64144, or a homolog or fragment thereof, or a homolog thereof being depicted in Table I, application no. 20, column 8, in the same line as SEQ ID NO. 64144, preferably the coding region thereof, a homolog or a fragment thereof; which are operable linked.

[102618] In an embodiment thereof the expression cassette comprises in addition c) a nucleic acid molecule encoding a transit peptide, especially a plastidal transit petide in case in Table I, application no. 20, in column 6 in the same line as SEQ ID NO. 64144 is depicted, plastidic is mentioned, or a mitochondrial transit peptide in case in Table I, application no. 20, in column 6 in the same line as SEQ ID NO. 64144 is depicted, mitochondric is mentioned; which is operable linked, too.

[102619] In another embodiment thereof the expression cassette does not comprise an additional transit peptide in case in Table I, application no. 20, in column 6 in the same line as SEQ ID NO. 64144 is depicted, non-targeted is mentioned.

[102620] In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 120309, or a homolog or fragment thereof, or a homolog thereof being depicted in Table I, application no. 20, column 8, in the same line as SEQ ID NO. 120309, preferably the coding region thereof, a homolog or a fragment thereof; which are operable linked.

[102621] In an embodiment thereof the expression cassette comprises in addition c) a nucleic acid molecule encoding a transit peptide, especially a plastidal transit petide in case in Table I, application no. 20, in column 6 in the same line as SEQ ID NO. 120309 is depicted, plastidic is mentioned, or a mitochondrial transit peptide in case in Table I, application no. 20, in column 6 in the same line as SEQ ID NO. 120309 is depicted, mitochondric is mentioned; which is operable linked, too.

[102622] In another embodiment thereof the expression cassette does not comprise an additional transit peptide in case in Table I, application no. 20, in column 6 in the same line as SEQ ID NO. 120309 is depicted, non-targeted is mentioned.

[102623] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 157717, or a homolog or fragment thereof, or a homolog thereof being depicted in Table I, application no. 20, column 8, in the same line as SEQ ID NO. 157717, preferably the coding region thereof, a homolog or a fragment thereof; which are operable linked.

[102624] In an embodiment thereof the expression cassette comprises in addition c) a nucleic acid molecule encoding a transit peptide, especially a plastidal transit petide in case in Table I, application no. 20, in column 6 in the same line as SEQ ID NO. 157717 is depicted, plastidic is mentioned, or a mitochondrial transit peptide in case in Table I, application no. 20, in column 6 in the same line as SEQ ID NO. 157717 is depicted, mitochondric is mentioned; which is operable linked, too.

[102625] In another embodiment thereof the expression cassette does not comprise an additional transit peptide in case in Table I, application no. 20, in column 6 in the same line as SEQ ID NO. 157717 is depicted, non-targeted is mentioned.

[102626] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 157832, or a homolog or fragment thereof, or a homolog thereof being depicted in Table I, application no. 20, column 8, in the same line as SEQ ID NO. 157832, preferably the coding region thereof, a homolog or a fragment thereof; which are operable linked.

[102627] In an embodiment thereof the expression cassette comprises in addition c) a nucleic acid molecule encoding a transit peptide, especially a plastidal transit petide in case in Table I, application no. 20, in column 6 in the same line as SEQ ID NO. 157832 is depicted, plastidic is mentioned, or a mitochondrial transit peptide in case in Table I, application no. 20, in column 6 in the same line as SEQ ID NO. 157832 is depicted, mitochondric is mentioned; which is operable linked, too.

[102628] In another embodiment thereof the expression cassette does not comprise an additional transit peptide in case in Table I, application no. 20, in column 6 in the same line as SEQ ID NO. 157832 is depicted, non-targeted is mentioned.

[102629] In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 157858, or a homolog or fragment thereof, or a homolog thereof being depicted in Table I, application no. 20, column 8, in the same line as SEQ ID NO. 157858, preferably the coding region thereof, a homolog or a fragment thereof; which are operable linked.

[102630] In an embodiment thereof the expression cassette comprises in addition c) a nucleic acid molecule encoding a transit peptide, especially a plastidal transit petide in case in Table I, application no. 20, in column 6 in the same line as SEQ ID NO. 157858 is depicted, plastidic is mentioned, or a mitochondrial transit peptide in case in Table I, application no. 20, in column 6 in the same line as SEQ ID NO. 157858 is depicted, mitochondric is mentioned; which is operable linked, too.

[102631] In another embodiment thereof the expression cassette does not comprise an additional transit peptide in case in Table I, application no. 20, in column 6 in the same line as SEQ ID NO. 157858 is depicted, non-targeted is mentioned.

[102632] In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 97631, or a homolog or fragment thereof, or a homolog thereof being depicted in Table I, application no. 20, column 8, in the same line as SEQ ID NO. 97631, preferably the coding region thereof, a homolog or a fragment thereof; which are operable linked.

[102633] In an embodiment thereof the expression cassette comprises in addition c) a nucleic acid molecule encoding a transit peptide, especially a plastidal transit petide in case in Table I, application no. 20, in column 6 in the same line as SEQ ID NO. 97631 is depicted, plastidic is mentioned, or a mitochondrial transit peptide in case in Table I, application no. 20, in column 6 in the same line as SEQ ID NO. 97631 is depicted, mitochondric is mentioned; which is operable linked, too.

[102634] In another embodiment thereof the expression cassette does not comprise an additional transit peptide in case in Table I, application no. 20, in column 6 in the same line as SEQ ID NO. 97631 is depicted, non-targeted is mentioned.

[102635] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 157862, or a homolog or fragment thereof, or a homolog thereof being depicted in Table I, application no. 20, column 8, in the same line as SEQ ID NO. 157862, preferably the coding region thereof, a homolog or a fragment thereof; which are operable linked.

[102636] In an embodiment thereof the expression cassette comprises in addition c) a nucleic acid molecule encoding a transit peptide, especially a plastidal transit petide in case in Table I, application no. 20, in column 6 in the same line as SEQ ID NO. 157862 is depicted, plastidic is mentioned, or a mitochondrial transit peptide in case in Table I, application no. 20, in column 6 in the same line as SEQ ID NO. 157862 is depicted, mitochondric is mentioned; which is operable linked, too.

[102637] In another embodiment thereof the expression cassette does not comprise an additional transit peptide in case in Table I, application no. 20, in column 6 in the same line as SEQ ID NO. 157862 is depicted, non-targeted is mentioned.

[102638] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 129546, or a homolog or fragment thereof, or a homolog thereof being depicted in Table I, application no. 20, column 8, in the same line as SEQ ID NO. 129546, preferably the coding region thereof, a homolog or a fragment thereof; which are operable linked.

[102639] In an embodiment thereof the expression cassette comprises in addition c) a nucleic acid molecule encoding a transit peptide, especially a plastidal transit petide in case in Table I, application no. 20, in column 6 in the same line as SEQ ID NO. 129546 is depicted, plastidic is mentioned, or a mitochondrial transit peptide in case in Table I, application no. 20, in column 6 in the same line as SEQ ID NO. 129546 is depicted, mitochondric is mentioned; which is operable linked, too.

[102640] In another embodiment thereof the expression cassette does not comprise an additional transit peptide in case in Table I, application no. 20, in column 6 in the same line as SEQ ID NO. 129546 is depicted, non-targeted is mentioned.

[102641] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 113647, or a homolog or fragment thereof, or a homolog thereof being depicted in Table I, application no. 20, column 8, in the same line as SEQ ID NO. 113647, preferably the coding region thereof, a homolog or a fragment thereof; which are operable linked.

[102642] In an embodiment thereof the expression cassette comprises in addition c) a nucleic acid molecule encoding a transit peptide, especially a plastidal transit petide in case in Table I, application no. 20, in column 6 in the same line as SEQ ID NO. 113647 is depicted, plastidic is mentioned, or a mitochondrial transit peptide in case in Table I, application no. 20, in column 6 in the same line as SEQ ID NO. 113647 is depicted, mitochondric is mentioned; which is operable linked, too.

[102643] In another embodiment thereof the expression cassette does not comprise an additional transit peptide in case in Table I, application no. 20, in column 6 in the same line as SEQ ID NO. 113647 is depicted, non-targeted is mentioned.

[102644] In an embodiment the present invention relates to an expresssion cassette comprising a) a promoter, preferably a promoter selected from the group consisting of Big35S, PCUbi, Super and USP, in particular Big35S; and b) the nucleic acid SEQ ID NO. 158033, or a homolog or fragment thereof, or a homolog thereof being depicted in Table I, application no. 20, column 8, in the same line as SEQ ID NO. 158033, preferably the coding region thereof, a homolog or a fragment thereof; which are operable linked.

[102645] In an embodiment thereof the expression cassette comprises in addition c) a nucleic acid molecule encoding a transit peptide, especially a plastidal transit petide in case in Table I, application no. 20, in column 6 in the same line as SEQ ID NO. 158033 is depicted, plastidic is mentioned, or a mitochondrial transit peptide in case in Table I, application no. 20, in column 6 in the same line as SEQ ID NO. 158033 is depicted, mitochondric is mentioned; which is operable linked, too.

[102646] In another embodiment thereof the expression cassette does not comprise an additional transit peptide in case in Table I, application no. 20, in column 6 in the same line as SEQ ID NO. 158033 is depicted, non-targeted is mentioned.

[102647] In an embodiment the present invention relates to an expresssion cassette comprising a) a promoter, preferably a promoter selected from the group consisting of Big35S, PCUbi, Super and USP, in particular p-Super; and b) the nucleic acid SEQ ID NO. 125348, or a homolog or fragment thereof, or a homolog thereof being depicted in Table I, application no. 20, column 8, in the same line as SEQ ID NO. 125348, preferably the coding region thereof, a homolog or a fragment thereof; which are operable linked.

[102648] In an embodiment thereof the expression cassette comprises in addition c) a nucleic acid molecule encoding a transit peptide, especially a plastidal transit petide in case in Table I, application no. 20, in column 6 in the same line as SEQ ID NO. 125348 is depicted, plastidic is mentioned, or a mitochondrial transit peptide in case in Table I, application no. 20, in column 6 in the same line as SEQ ID NO. 125348 is depicted, mitochondric is mentioned; which is operable linked, too.

[102649] In another embodiment thereof the expression cassette does not comprise an additional transit peptide in case in Table I, application no. 20, in column 6 in the same line as SEQ ID NO. 125348 is depicted, non-targeted is mentioned.

[102650] [0165.1.7.20] to [0170.1.7.20] for the disclosure of these paragraphs see [0165.1.7.7] to [0170.1.7.7] above

[102651] The invention further provides a vector, comprising a nucleic acid molecule comprising 1) a nucleic acid molecule selected from the group consisting of:
[102652] (a) a nucleic acid molecule encoding a polypeptide shown in Table II, column 5 or 8, application no. 20, preferably shown in Table II A, application no. 20, in column 5, or in Table II A, application no. 20, column 8, or in Table II B, application no. 20, column 8, or a homolog or a fragment thereof;
[102653] (b) a nucleic acid molecule shown in Table I, application no. 20, column 5 or 8, preferably shown in Table I A, application no. 20, in column 5, or in Table I A, application no. 20, column 8, or in Table I B, application no. 20, column 8, preferably the coding region thereof, or a homolog or a fragment thereof;
[102654] (c) a nucleic acid molecule, which, as a result of the degeneracy of the genetic code, can be derived from a polypeptide sequence depicted in column 5 or 8 of Table II, application no. 20, preferably shown in Table II A, application no. 20, in column 5, or in Table II A, application no. 20, column 8, or in Table II B, application no. 20, column 8;
[102655] (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 20, preferably shown in Table I A, application no. 20, in column 5, or in Table I A, application no. 20, column 8, or in Table I B, application no. 20, column 8, preferably the coding region thereof;
[102656] (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
[102657] (f) a nucleic acid molecule which hybridizes with a nucleic acid molecule of (a), (b), (c), (d), or (e) under stringent hybridization conditions;
[102658] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
[102659] (h) a nucleic acid molecule encoding a polypeptide comprising the consensus sequence or one or more polypeptide motifs as shown in column 8 of Table IV, application no. 20;
[102660] (i) a nucleic acid molecule encoding a polypeptide having the activity represented by a protein as depicted in column 5 of Table II, application no. 20;
[102661] (j) a nucleic acid molecule comprising a polynucleotide which is obtained by amplifying a cDNA library or a genomic library using the primers in Table III, application no. 20, column 8;
[102662] and
[102663] (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
[102664] or a nucleic acid molecule comprising a sequence which is complementary thereto.
   (All the references to Tables I to IV are references to the corresponding lines of Tables I to IV.)

[102665] In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 20.

[102666] In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wildtype non-human organism or part thereof.

[102667] In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 20, and confers a generation or an increase of the respective fine chemical in a non-human organism or a part thereof as compared to a corresponding wild-type non-human organism or part thereof.

[102668] In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

[102669] In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 20.

[102670] In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[102671] In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 20, and confers a generation or an increase of the respective fine chemical in a non-human organism or a part thereof as compared to a corresponding wild-type non-human organism or part thereof.

[102672] In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 20, in the same line as of the respective nucleic acid molecule according to 1) “plastidic” is depicted, or encoding a mitochondrial transit peptide, especially in case in column 6 of Table I, application no. 20, in the same line as of the respective nucleic acid molecule according to 1) “mitochondric” is depicted, which are operable linked.

[102673] In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 20, in the same line as of the respective nucleic acid molecule according to 1) “non-targeted” is depicted.

[102674] In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 20, in the same line as of the respective nucleic acid molecule according to 1) “plastidic” is depicted, or encoding a mitochondrial transit peptide, especially in case in column 6 of Table I, application no. 20, in the same line as of the respective nucleic acid molecule according to 1) “mitochondric” is depicted, which are operable linked.

[102675] In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operable linked; a nucleic acid molecule encoding a transit peptide, especially in case in column 6 of Table I, application no. 20, in the same line as of the respective nucleic acid molecule according to 1) “non-targeted” is depicted.

[102676] In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 20, in the same line as of the respective nucleic acid molecule according to 1) “plastidic” is depicted, or encoding a mitochondrial transit peptide, especially in case in column 6 of Table I, application no. 20, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 20 (in the same line).

[102677] In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 20, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 20.

[102678] In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 20, in the same line as of the respective nucleic acid molecule according to 1) “plastidic” is depicted, or encoding a mitochondrial transit peptide, especially in case in column 6 of Table I, application no. 20, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[102679] In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 20, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[102680] In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 20, in the same line as of the respective nucleic acid molecule according to 1) “plastidic” is depicted, or encoding a mitochondrial transit peptide, especially in case in column 6 of Table I, application no. 20, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 20 and confers a generation or an increase of the respective fine chemical in a non-human organism or a part thereof as compared to a corresponding wild-type non-human organism or part thereof.

[102681] In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 20, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 20 (in the same line) and confers a generation or an increase of the respective fine chemical in a non-human organism or a part thereof as compared to a corresponding wild-type non-human organism or part thereof.

[102682] In a preferred embodiment of said vector said nucleic acid molecule according to 1 (a), (b), (c), (d), (e), (f), (g), (h), (i), (j) or (k), said promoter and optionally said nucleic acid molecule encoding a transit peptide are encompassed in an expression cassette. Accordingly in an embodiment the invention provides a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

[102683] The invention further provides a vector comprising a nucleic acid molecule comprising 1) a nucleic acid molecule selected from the group consisting of:
[102684] (a) a nucleic acid molecule encoding a polypeptide shown in Table II, column 5 or 8, application no. 20, preferably shown in Table II A, application no. 20, in column 5, or in Table II A, application no. 20, column 8, or in Table II B, application no. 20, column 8, or a homolog or a fragment thereof;
[102685] (b) a nucleic acid molecule shown in Table I, application no. 20, column 5 or 8, preferably shown in Table I A, application no. 20, in column 5, or in Table I A, application no. 20, column 8, or in Table I B, application no. 20, column 8, preferably the coding region thereof, or a homolog or a fragment thereof;
[102686] (c) a nucleic acid molecule, which, as a result of the degeneracy of the genetic code, can be derived from a polypeptide sequence depicted in column 5 or 8 of Table II, application no. 20, preferably shown in Table II A, application no. 20, in column 5, or in Table II A, application no. 20, column 8, or in Table II B, application no. 20, column 8;
[102687] (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 20, preferably shown in Table I A, application no. 20, in column 5, or in Table I A, application no. 20, column 8, or in Table I B, application no. 20, column 8, preferably the coding region thereof;
[102688] (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c), or (d);
[102689] (f) a nucleic acid molecule which hybridizes with a nucleic acid molecule of (a), (b), (c), (d), or (e) under stringent hybridization conditions;
[102690] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
[102691] (h) a nucleic acid molecule encoding a polypeptide comprising the consensus sequence or one or more polypeptide motifs as shown in column 8 of Table IV, application no. 20;
[102692] (i) a nucleic acid molecule encoding a polypeptide having the activity represented by a protein as depicted in column 5 of Table II, application no. 20;
[102693] (j) a nucleic acid molecule comprising a polynucleotide which is obtained by amplifying a cDNA library or a genomic library using the primers in Table III, application no. 20, column 8;
[102694] and
[102695] (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment thereof having at least 15 nt, preferably 20 nt, 30 nt, 50 nt, 100 nt, 200 nt, 500 nt, 1000 nt, 1500 nt, 2000 nt or 3000 nt of the nucleic acid molecule complementary to a nucleic acid sequence characterized in (a), (b), (c), (d), (e), (f), (g), (h), (i) or (j);
[102696] or a nucleic acid molecule comprising a sequence which is complementary thereto;
   whereby, preferably, the nucleic acid molecule according to 1 (a), (b), (c), (d), (e), (f), (g), (h), (i), (j) or (k) distinguishes over the sequence depicted in Table I A and/or I B, application no. 20, column 5 or 8, or the coding region thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 20, column 5 or 8, or the coding region thereof, respectively. In another embodiment, the nucleic acid molecule of the present invention, preferably the coding region thereof, is 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 20, column 5 or 8, or the coding region thereof, respectively. In a further embodiment the nucleic acid molecule does not encode the polypeptide sequence shown in Table II A and/or II B, application no. 20, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 20, column 5 or 8 and does not encode a protein of the sequence shown in Table II A and/or II B, application no. 20, column 5 or 8. Accordingly, in one embodiment, the protein encoded by a sequence of a nucleic acid according to (a), (b), (c), (d), (e), (f), (g), (h), (i), (j) or (k) does not consist of the sequence shown in Table I A and/or I B, application no. 20, column 5 and 8, or the coding region thereof. In a further embodiment, the protein of the present invention is at least 30%,40%, 50%, 60%, 70%, 80%, 90%, 95% identical to protein sequence depicted in Table II A and/or II B, application no. 20, column 5 or 8 but less than 100%, preferably less than 99.999%, 99.99% or 99.9%, more preferably less than 99%, 985, 97%, 96% or 95% identical to the sequence shown in Table II A and/or II B, application no. 20, column 5 or 8.
   (All the references to Tables I to IV are references to the corresponding lines of Tables I to IV.)

[102697] In a preferred embodiment of said vector said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 20.

[102698] In another preferred embodiment of said vector said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wildtype non-human organism or part thereof.

[102699] In another preferred embodiment of said vector said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 20, and confers a generation or an increase of the respective fine chemical in a non-human organism or a part thereof as compared to a corresponding wild-type non-human organism or part thereof.

[102700] In another embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

[102701] In a preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 20.

[102702] In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[102703] In another preferred embodiment thereof said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 20, and confers a generation or an increase of the respective fine chemical in a non-human organism or a part thereof as compared to a corresponding wild-type non-human organism or part thereof.

[102704] In another embodiment thereof said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 20, in the same line as of the respective nucleic acid molecule according to 1) “plastidic” is depicted, or encoding a mitochondrial transit peptide, especially in case in column 6 of Table I, application no. 20, in the same line as of the respective nucleic acid molecule according to 1) “mitochondric” is depicted, which are operable linked.

[102705] In another embodiment thereof said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 20, in the same line as of the respective nucleic acid molecule according to 1) “non-targeted” is depicted.

[102706] In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 20, in the same line as of the respective nucleic acid molecule according to 1) “plastidic” is depicted, or encoding a mitochondrial transit peptide, especially in case in column 6 of Table I, application no. 20, in the same line as of the respective nucleic acid molecule according to 1) “mitochondric” is depicted, which are operable linked.

[102707] In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 20, in the same line as of the respective nucleic acid molecule according to 1) “non-targeted” is depicted.

[102708] In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 20, in the same line as of the respective nucleic acid molecule according to 1) “plastidic” is depicted, or encoding a mitochondrial transit peptide, especially in case in column 6 of Table I, application no. 20, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 20.

[102709] In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 20, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in column 5 of Table II, application no. 20.

[102710] In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 20, in the same line as of the respective nucleic acid molecule according to 1) “plastidic” is depicted, or encoding a mitochondrial transit peptide, especially in case in column 6 of Table I, application no. 20, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[102711] In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 20, in the same line as of the respective nucleic acid molecule according to 1) “non-targeted” is depicted; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[102712] In another embodiment thereof said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 20, in the same line as of the respective nucleic acid molecule according to 1) “plastidic” is depicted, or encoding a mitochondrial transit peptide, especially in case in column 6 of Table I, application no. 20, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 20 and confers a generation or an increase of the respective fine chemical in a non-human organism or a part thereof as compared to a corresponding wild-type non-human organism or part thereof.

[102713] In another embodiment thereof said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP, which are operatively linked; a nucleic acid molecule encoding a transit peptide, especially in case in column 6 of Table I, application no. 20, in the same line as of the respective nucleic acid molecule according to 1) “non-targeted” is depicted; and wherein said nucleic acid molecule according to 1 (a), (b), (c), (d), (e), (f), (g), (h), (i), (j) or (k) encodes a polypeptide which has the activity of the respective polypeptide represented by a protein as depicted in column 5 of Table II application no. 20 and confers a generation or an increase of the respective fine chemical in a non-human organism or a part thereof as compared to a corresponding wild-type non-human organism or part thereof.

[102714] [0181.1.7.20] to [0209.1.7.20] for the disclosure of these paragraphs see [0181.1.7.7] to [0209.1.7.7] above

[102715] The genes of the invention, coding for an activity selected from the group consisting of 3-deoxy-7-phosphoheptulonate synthase, acid shock protein, amino-acid acetyltransferase, assembly protein for periplasmic nitrate reductase, At2g45420-protein, At5g43630-protein, ATP-dependent RNA helicase, b0833-protein, b1259-protein, b1445-protein, b1670-protein, b1930-protein, b2399-protein, b2653-protein, b3644-protein, cell division protein, cyclin D, cysteine synthase A, dihydroxyacetone kinase, electron transport complex protein, ethanolamine utilization protein, exonuclease subunit, fumarylacetoacetate hydrolase, GDP-mannose 4,6-dehydratase, inner membrane protein, ion-transport protein, L-asparaginase, MADS box protein transcription factor, mechanosensitive ion channel, metal-dependent hydrolase, methionine permease, O-antigen chain length determinant, ornithine decarboxylase, orotidine-5′-phosphate decarboxylase, peptide chain release factor, phospho-2-dehydro-3-deoxyheptonate aldolase, phosphoadenosine phosphosulfate reductase, phosphoglycolate phosphatase, pyrimidine-specific ribonucleoside hydrolase, pyruvate kinase, RNA-binding protein, S-adenosyl-L-methionine-dependent methyltransferase, serine protease, short-chain alcohol dehydrogenase family, sigma-54 dependent response regulator, spermidine synthase, sugar transport system permease protein, transcription factor, transcriptional regulator, YFL019C-protein, YHL039W-protein, YHR022C-A-protein, YJL127W-A-protein, ylr157c-protein, ynl321w-protein, ypr170c-protein, and YPR172W-protein are also called “FCRP genes”.

[102716] [0211.1.7.20] to [0225.1.7.20] for the disclosure of these paragraphs see [0211.1.7.7] [0225.1.7.7] above

[102717] In addition to the sequence mentioned in Table I, application no. 20, column 5 or 8, preferably the coding region thereof, or its homologs or fragments, it may be advantageous additionally to express and/or mutate further genes in the non-human organisms. Especially advantageously, additionally at least one further gene of the biosynthetic pathway of the fine chemical is expressed in the non-human organisms such as plants or microorganisms. It is also possible that the regulation of the natural genes has been modified advantageously so that the gene and/or its gene product is no longer subject to the regulatory mechanisms which exist in the non-human organisms. This leads to an increased synthesis of the respective fine chemical since, for example, feedback regulations no longer exist to the same extent or not at all. In addition it might be advantageously to combine the nucleic acids sequences of the invention containing the sequences shown in the respective line in Table I, application no. 20, column 5 or 8, preferably the coding region thereof, or homologs or fragments thereof, with genes which generally support or enhance the growth or yield of the target non-human organisms, for example genes which lead to faster growth rate of non human organism like microorganisms or plants or genes which produce stress-, pathogen-, or herbicide-resistant plants.

[102718] In a further embodiment of the process of the invention, non-human organisms are grown, in which there is in addition to the expression of the nucleic acid molecule according to the invention simultaneous expression of at least one nucleic acid molecule or one of the genes which code for proteins involved in the waxes metabolism, in particular in long chain alkanes and/or VLCFAs synthesis, especially genes selected from the group coding for fatty acid elongasen, or a polypeptide having a very long chain fatty acid acyl (VLCFA) CoA synthase activity and/or genes selected from the group coding for Ketoacetyl-synthase (=VLCFA-synthase), e.g. FAE1 (AT4G34520) from A. thaliana, beta-Ketoacyl-CoA-reductase, beta-Hydroxyacyl-CoA-dehydratase, 2-trans-Enoyl-CoA-reductase and/or 3-ketoacyl-CoA synthases involved in VLCFA-biosynthesis and/or having the activity of a annotated gene selected from the group comprising AT1G04220 (KCS2), AT1G07720 (KCS3), AT1G19440 (KCS4), AT1G71160 (KCS7), AT2G15090 (KCS8), AT2G16280 (KCS9), AT2G26640 (KCSll), AT2G28630 (KCS12), AT2G46720 (KSC13), AT3G10280 (KCS14), AT3G52160 (KCS15), AT4G34250 (KCS16), AT4G34510 (KCS17), AT4G34520 (KCS18), AT5G04530 (KCS19), AT5G43760 (KCS20) and AT5G49070 (KCS21).

[102719] [0228.1.7.20] to [0239.1.7.20] for the disclosure of these paragraphs see [0228.1.7.7] to [0239.1.7.7] above

[102720] In an embodiment of the present invention the nucleic acid construct of the present invention comprises a nucleic acid molecule as depicted in the respective line in Table I, application no. 20, column 5 or 8, preferably the coding region thereof, a fragment or a homolog thereof, and a plant promoter, such as the Big 35S promoter, the DC3 promoter, the PC Ubi promoter, the LegB4-promoter, the Super promoter, the USP promoter or the like. In another embodiment the nucleic acid construct of the present invention comprises a nucleic acid molecule as depicted in the respective line in Table I, application no. 20, column 5 or 8, preferably the coding region thereof, a fragment or a homolog thereof, and a plant promoter such as, for example, the USP, the LegB4-, the DC3 promoter or the ubiquitin promoter from parsley or other herein mentioned promoters. In a further embodiment the nucleic acid construct of the present invention comprises a nucleic acid molecule as depicted in the respective line in Table I, application no. 20, column 5 or 8, preferably the coding region thereof, a fragment or a homolog thereof, and a plant promoter, such as the Big 35S promoter, the PC Ubi promoter, the Super promoter, the USP promoter or the like. In addition different terminators may advantageously be used in these nucleic acid constructs; examples for transcriptional termination are polyadenylation signals.

[102721] [0241.1.7.20] to [0245.1.7.20] for the disclosure of these paragraphs see [0241.1.7.7] to [0245.1.7.7] above

[102722] Other preferred sequences for use in operable linkage in gene expression constructs are targeting sequences, which are required for targeting the gene product into specific cell compartments (for a review, see Kermode, Crit. Rev. Plant Sci. 15 (4), 285 (1996) and references cited therein), for example into the vacuole, the nucleus, all types of plastids, such as amyloplasts, chloroplasts, chromoplasts, the extracellular space, the mitochondria, the endoplasmic reticulum, elaioplasts, peroxisomes, glycosomes, and other compartments of cells or extracellular; preferred are sequences, which are involved in targeting to plastids as mentioned above. Sequences, which must be mentioned in this context are, in particular, the signal-peptide- or transit-peptide-encoding sequences which are known per se. For example, plastid transit-peptide-encoding sequences enable the targeting of the expression product into the plastids of a plant cell. Targeting sequences are also known for eukaryotic and to a lower extent for prokaryotic organisms and can advantageously be operable linked with the nucleic acid molecule of the present invention as shown in Table I, application no. 20, columns 5 and 8, preferably the coding region thereof, or homologs or fragments thereof, and described herein to achieve an expression in one of said compartments or extracellular.

[102723] [0247.1.7.20] to [0249.1.7.20] for the disclosure of these paragraphs see [0247.1.7.7] to [0249.1.7.7] above

[102724] Preferred promoters are in particular those which bring about gene expression in tissues and organs, such as leaves, roots, seeds, pollen or xylem. Examples of tissue preferred or organ preferred promoters include, but are not limited to fruit-preferred, ovule-preferred, male tissue-preferred, seed-preferred, pollen-preferred integument-preferred, tuber-preferred, stalk-preferred, pericarp-preferred, leaf-preferred, stigma-preferred, pollen-preferred, anther-preferred, petal-preferred, sepal-preferred, pedicel-preferred, silique-preferred, stem-preferred, root-preferred promoters, and the like. Especially those promoters are preferred which bring about gene expression in tissues and organs in which the biosynthesis of waxes and/or long chain alkanes takes place, like in seed cells, such as endosperm cells and cells of the developing embryo. Seed promoters are preferentially expressed during seed development and/or germination. For example, seed preferred promoters can be embryo-preferred, endosperm preferred and seed coat-preferred (see Thompson et al., BioEssays 10, 108 (1989)).

[102725] Examples of seed preferred promoters include, but are not limited to, cellulose synthase (celA), Cim1, gamma-zein, globulin-1, maize 19 kD zein (cZ19B1), and the like. Other suitable promoters are the oilseed rape napin gene promoter (U.S. Pat. No. 5,608,152), the Vicia faba USP promoter (Baeumlein et al., Mol Gen Genet, 225 (3), 459 (1991)), the Arabidopsis oleosin promoter (WO 98/45461), the Phaseolus vulgaris phaseolin promoter (U.S. Pat. No. 5,504,200), the Brassica Bce4 promoter (WO 91/13980), the bean arcs promoter, the carrot DcG3 promoter, or the Legumin B4 promoter (LeB4) (Baeumlein et al., Plant Journal, 2 (2), 233 (1992)), and promoters which bring about the seed-specific expression in monocotyledonous plants such as maize, barley, wheat, rye, rice and the like. Advantageous seed-specific promoters are the sucrose binding protein promoter (WO 00/26388), the phaseolin promoter and the napin promoter. Suitable promoters which must be considered are the barley Ipt2 or Ipt1 gene promoter (WO 95/15389 and WO 95/23230), and the promoters described in WO 99/16890 (promoters from the barley hordein gene, the rice glutelin gene, the rice oryzin gene, the rice prolamin gene, the wheat gliadin gene, the wheat glutelin gene, the maize zein gene, the oat glutelin gene, the sorghum kasirin gene and the rye secalin gene). Further suitable promoters are Amy32b, Amy 6-6 and Aleurain (U.S. Pat. No. 5,677,474), Bce4 (oilseed rape) (U.S. Pat. No. 5,530,149), glycinin (soya) (EP 571 741], phosphoenolpyruvate carboxylase (soya) (JP 06/62870), ADR12-2 (soya) (WO 98/08962), isocitrate lyase (oilseed rape) (U.S. Pat. No. 5,689,040]) or α-amylase (barley) (EP 781 8499. Other promoters which are available for the expression of genes in plants are leaf-specific promoters such as those described in DE-A 19 644 478 or light-regulated promoters such as, for example, the pea petE promoter.

[102726] [0251.1.7.20] to [0266.1.7.20] for the disclosure of this paragraph see [0251.1.7.7] to [0266.1.7.7] above

[102727] An embodiment of the invention relates to a vector where the nucleic acid molecule according to the invention is linked operably to regulatory sequences which permit the expression in a prokaryotic or eukaryotic host. A further embodiment of the invention relates to a vector in which a nucleic acid sequence encoding one of the polypeptides shown in the respective line in Table II, application no. 20, columns 5 or 8, or homologs or fragments thereof, is functionally linked to a targeting sequence such as a plastidial or mitochondrial targeting sequence. A further embodiment of the invention relates to a vector in which a nucleic acid sequence encoding one of the polypeptides shown in the respective line in Table II, application no. 20, columns 5 or 8, or homologs or fragments thereof, is functionally linked to (a) regulatory sequence which permit the expression in an organelle, such as plastids or mitochondria, preferably plastids.

[102728] A further embodiment of the invention relates to a vector which comprises a nucleic acid sequence encoding one of the polypeptides shown in the respective line in Table II, application no. 20, columns 5 or 8, or homologs or fragments thereof, but no additional targeting sequence.

[102729] [0268.1.7.20] to [0273.1.7.20] for the disclosure of these paragraphs see [0268.1.7.7] to [0273.1.7.20] above

[102730] A further object of the invention relates to the use of a nucleic acid construct, e.g. an expression cassette, comprising one or more nucleic acid molecules encoding one or more polypeptides as depicted in the respective line(s) in Table II, application no. 20, columns 5 or 8, or homologs or fragments thereof, or comprising one or more nucleic acid molecules as depicted in the respective line(s) in Table I, application no. 20, columns 5 or 8, preferably the coding region thereof, or homologs or fragments thereof, or comprising a nucleic acid molecule hybridizing therewith for the transformation of a microorganism or a plant cell, plant tissues or a part of a plant

[102731] In doing so, depending on the choice of promoter, the nucleic acid molecules shown in the respective line in Table I, application no. 20, column 5 or 8, preferably the coding region thereof, or homologs or fragments thereof, or the nucleic acid molecules encoding a polypeptide as depicted in the respective line in Table II, application no. 20, column 5 or 8, or homologs or fragments thereof, can be expressed in the whole plant or specifically e.g. in the leaves, in the seeds, the nodules, in roots, in the stem or other parts of the plant. Those transgenic plants overproducing sequences, e.g. as depicted in the respective line in Table I, application no. 20, column 5 or 8, preferably the coding region thereof, or homologs or fragments thereof, the plant cell, plant tissues, plants, the reproductive material thereof, or parts thereof are a further object of the present invention.

[102732] Within the framework of the present invention, the production of the hentriacontane is due to the generation or over-expression of one or more polypeptides as depicted in the respective line(s) in Table II, application no. 20, column 5 or 8, or homologs or fragments thereof, or encoded by the corresponding nucleic acid molecules as depicted in the respective line(s) in Table I, application no. 20, column 5 or 8, preferably the coding region thereof, or homologs or fragments thereof, in the non-human organism according to the invention, advantageously in the transgenic microorganism or plant according to the invention, at least for the duration of at least one plant generation.

[102733] A constitutive expression of the polypeptide as depicted in the respective line in Table II, application no. 20, column 5 or 8, or homologs or fragments thereof, or encoded by the respective nucleic acid molecule as depicted in the respective line in Table I, application no. 20, column 5 or 8, preferably the coding region thereof, or homologs or fragments thereof, may be advantageous. On the other hand, an inducible expression may also appear desirable. Expression of the polypeptide sequences of the invention can be either directed to the cytosol or to the organelles, such as plastids or mitochondria, preferably the plastids of the host cells, preferably the plant cells, or non-targeted.

[102734] for the disclosure of this paragraph see [0278.1.7.7] above

[102735] In a further embodiment the invention relates to a transgenic non-human organisms such as transgenic microorganism or transgenic plant comprising a nucleic acid molecule comprising
[102736] [1) a nucleic acid molecule selected from the group consisting of
   [102737] (a) a nucleic acid molecule encoding a polypeptide shown in Table II, application no. 20, column 5 or 8, preferably shown in Table II A, application no. 20, column 5, or in Table II A, application no. 20, column 8, or in Table II B, application no. 20, column 8, or a homolog or a fragment thereof;
   [102738] (b) a nucleic acid molecule shown in Table I, application no. 20, column 5 or 8, preferably shown in Table I A, application no. 20, column 5, or in Table I A, application no. 20, column 8, or in Table I B, application no. 20, column 8, preferably the coding region thereof, or a homolog or a fragment thereof;
   [102739] (c) a nucleic acid molecule, which, as a result of the degeneracy of the genetic code, can be derived from a polypeptide sequence depicted in column 5 or 8 of Table II, application no. 20, preferably shown in Table II A, application no. 20, column 5, or in Table II A, application no. 20, column 8, or in Table II B, application no. 20, column 8;
   [102740] (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 20, preferably shown in Table I A, application no. 20, column 5, or in Table I A, application no. 20, column 8, or in Table I B, application no. 20, column 8, preferably the coding region thereof;
   [102741] (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   [102742] (f) a nucleic acid molecule which hybridizes with a nucleic acid molecule of (a), (b), (c), (d) or (e) under stringent hybridization conditions;
   [102743] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   [102744] (h) a nucleic acid molecule encoding a polypeptide comprising the consensus sequence or one or more polypeptide motifs as shown in column 8 of Table IV, application no. 20;
   [102745] (i) a nucleic acid molecule encoding a polypeptide having the activity represented by a respective protein as depicted in column 5 of Table II, application no. 20;
   [102746] (j) a nucleic acid molecule comprising a polynucleotide which is obtained by amplifying a cDNA library or a genomic library using the primers in Table III, application no. 20, column 8;
   [102747] and
   [102748] (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   [102749] or a nucleic acid molecule comprising a sequence which is complementary thereto;
      and, if desired
[102750] 2) nucleic acid molecule encoding a transit peptide, preferably encoding a plastidal transit peptide or a mitochondrial transit peptide;
   which are operable linked;]
   or comprising an expression cassette comprising
[102751] [0) a promoter, preferably a promoter selected from the group consisting of Big35S, PCUbi, Super and USP; and
[102752] 1) a nucleic acid molecule selected from the group consisting of
   [102753] (a) a nucleic acid molecule encoding a polypeptide shown in Table II, application no. 20, column 5 or 8, preferably shown in Table II A, application no. 20, column 5, or in Table II A, application no. 20, column 8, or in Table II B, application no. 20, column 8, or a homolog or a fragment thereof;
   [102754] (b) a nucleic acid molecule shown in Table I, application no. 20, column 5 or 8, preferably shown in Table I A, application no. 20, column 5, or in Table I A, application no. 20, column 8, or in Table I B, application no. 20, column 8, preferably the coding region thereof, or a homolog or a fragment thereof;
   [102755] (c) a nucleic acid molecule, which, as a result of the degeneracy of the genetic code, can be derived from a polypeptide sequence depicted in column 5 or 8 of Table II, application no. 20, preferably shown in Table II A, application no. 20, column 5, or in Table II A, application no. 20, column 8, or in Table II B, application no. 20, column 8;
   [102756] (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 20, preferably shown in Table I A, application no. 20, column 5, or in Table I A, application no. 20, column 8, or in Table I B, application no. 20, column 8, preferably the coding region thereof;
   [102757] (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   [102758] (f) a nucleic acid molecule which hybridizes with a nucleic acid molecule of (a), (b), (c), (d) or (e) under stringent hybridization conditions;
   [102759] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   [102760] (h) a nucleic acid molecule encoding a polypeptide comprising the consensus sequence or one or more polypeptide motifs as shown in column 8 of Table IV, application no. 20;
   [102761] (i) a nucleic acid molecule encoding a polypeptide having the activity represented by a respective protein as depicted in column 5 of Table II, application no. 20;
   [102762] (j) a nucleic acid molecule comprising a polynucleotide which is obtained by amplifying a cDNA library or a genomic library using the primers in Table III, application no. 20, column 8;
   [102763] and
   [102764] (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   [102765] or a nucleic acid molecule comprising a sequence which is complementary thereto;
      and, if desired
[102766] 2) nucleic acid molecule encoding a transit peptide, preferably encoding a plastidal transit peptide or a mitochondrial transit peptide;
   which are operable linked;]
   or comprising or being transformed by a vector comprising
[102767] [1) a nucleic acid molecule selected from the group consisting of
   [102768] (a) a nucleic acid molecule encoding a polypeptide shown in Table II, application no. 20, column 5 or 8, preferably shown in Table II A, application no. 20, column 5, or in Table II A, application no. 20, column 8, or in Table II B, application no. 20, column 8, or a homolog or a fragment thereof;
   [102769] (b) a nucleic acid molecule shown in Table I, application no. 20, column 5 or 8, preferably shown in Table I A, application no. 20, column 5, or in Table I A, application no. 20, column 8, or in Table I B, application no. 20, column 8, preferably the coding region thereof, or a homolog or a fragment thereof;
   [102770] (c) a nucleic acid molecule, which, as a result of the degeneracy of the genetic code, can be derived from a polypeptide sequence depicted in column 5 or 8 of Table II, application no. 20, preferably shown in Table II A, application no. 20, column 5, or in Table II A, application no. 20, column 8, or in Table II B, application no. 20, column 8;
   [102771] (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 20, preferably shown in Table I A, application no. 20, column 5, or in Table I A, application no. 20, column 8, or in Table I B, application no. 20, column 8, preferably the coding region thereof;
   [102772] (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   [102773] (f) a nucleic acid molecule which hybridizes with a nucleic acid molecule of (a), (b), (c), (d) or (e) under stringent hybridization conditions;
   [102774] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   [102775] (h) a nucleic acid molecule encoding a polypeptide comprising the consensus sequence or one or more polypeptide motifs as shown in column 8 of Table IV, application no. 20;
   [102776] (i) a nucleic acid molecule encoding a polypeptide having the activity represented by a respective protein as depicted in column 5 of Table II, application no. 20;
   [102777] (j) a nucleic acid molecule comprising a polynucleotide which is obtained by amplifying a cDNA library or a genomic library using the primers in Table III, application no. 20, column 8;
   [102778] and
   [102779] (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   [102780] or a nucleic acid molecule comprising a sequence which is complementary thereto;]
      as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

[102781] In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 20.

[102782] In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[102783] In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 20 and confers a generation or an increase of the respective fine chemical in a non-human organism or a part thereof as compared to a corresponding wild-type non-human organism or part thereof.

[102784] In another embodiment of said transgenic non-human organism the expression cassette comprises 2) a nucleic acid molecule encoding a transit peptide, preferably encoding a plastidal transit peptide, especially in case in column 6 of Table I, application no. 20, in the same line as of the respective nucleic acid molecule according to 1) “plastidic” is depicted, or encoding a mitochondrial transit peptide, especially in case in column 6 of Table I, application no. 20, in the same line as of the respective nucleic acid molecule according to 1) “mitochondric” is depicted.

[102785] In another embodiment of said transgenic non-human organism the expression cassette does not comprise 2) a nucleic acid molecule encoding a transit peptide, especially in case in column 6 of Table I, application no. 20, in the same line as of the respective nucleic acid molecule according to 1) “non-targeted” is depicted.

[102786] In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

[102787] In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 20.

[102788] In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[102789] In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II, application no. 20, and confers a generation or an increase of the respective fine chemical in a non-human organism or a part thereof as compared to a corresponding wild-type non-human organism or part thereof.

[102790] In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 20, in the same line as of the respective nucleic acid molecule according to 1) “plastidic” is depicted, or encoding a mitochondrial transit peptide, especially in case in column 6 of Table I, application no. 20, in the same line as of the respective nucleic acid molecule according to 1) “mitochondric” is depicted, which are operable linked.

[102791] In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 20, in the same line as of the respective nucleic acid molecule according to 1) “non-targeted” is depicted, which are operable linked.

[102792] In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 20, in the same line as of the respective nucleic acid molecule according to 1) “plastidic” is depicted, or encoding a mitochondrial transit peptide, especially in case in column 6 of Table I, application no. 20, in the same line as of the respective nucleic acid molecule according to 1) “mitochondric” is depicted, which are operable linked.

[102793] In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 20, in the same line as of the respective nucleic acid molecule according to 1) “non-targeted” is depicted, which are operable linked.

[102794] In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 20, in the same line as of the respective nucleic acid molecule according to 1) “plastidic” is depicted, or encoding a mitochondrial transit peptide, especially in case in column 6 of Table I, application no. 20, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 20.

[102795] In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 20, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 20.

[102796] In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 20, in the same line as of the respective nucleic acid molecule according to 1) “plastidic” is depicted, or encoding a mitochondrial transit peptide, especially in case in column 6 of Table I, application no. 20, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[102797] In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 20, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[102798] In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 20, in the same line as of the respective nucleic acid molecule according to 1) “plastidic” is depicted, or encoding a mitochondrial transit peptide, especially in case in column 6 of Table I, application no. 20, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 20 and confers a generation or an increase of the respective fine chemical in a non-human organism or a part thereof as compared to a corresponding wild-type non-human organism or part thereof.

[102799] In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 20, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 20 (in the same line) and confers a generation or an increase of the respective fine chemical in a non-human organism or a part thereof as compared to a corresponding wild-type non-human organism or part thereof.

[102800] In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

[102801] In a further embodiment the invention relates to a transgenic non-human organisms such as transgenic microorganism or transgenic plant comprising a nucleic acid molecule comprising
[102802] [1) a nucleic acid molecule selected from the group consisting of
   [102803] (a) a nucleic acid molecule encoding a polypeptide shown in Table II, application no. 20, column 5 or 8, preferably shown in Table II A, application no. 20, column 5, or in Table II A, application no. 20, column 8, or in Table II B, application no. 20, column 8, or a homolog or a fragment thereof;
   [102804] (b) a nucleic acid molecule shown in Table I, application no. 20, column 5 or 8, preferably shown in Table I A, application no. 20, column 5, or in Table I A, application no. 20, column 8, or in Table I B, application no. 20, column 8, preferably the coding region thereof, or a homolog or a fragment thereof;
   [102805] (c) a nucleic acid molecule, which, as a result of the degeneracy of the genetic code, can be derived from a polypeptide sequence depicted in column 5 or 8 of Table II, application no. 20, preferably shown in Table II A, application no. 20, column 5, or in Table II A, application no. 20, column 8, or in Table II B, application no. 20, column 8;
   [102806] (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 20, preferably shown in Table I A, application no. 20, column 5, or in Table I A, application no. 20, column 8, or in Table I B, application no. 20, column 8, preferably the coding region thereof;
   [102807] (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   [102808] (f) a nucleic acid molecule which hybridizes with a nucleic acid molecule of (a), (b), (c), (d) or (e) under stringent hybridization conditions;
   [102809] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   [102810] (h) a nucleic acid molecule encoding a polypeptide comprising the consensus sequence or one or more polypeptide motifs as shown in column 8 of Table IV, application no. 20;
   [102811] (i) a nucleic acid molecule encoding a polypeptide having the activity represented by a respective protein as depicted in column 5 of Table II, application no. 20;
   [102812] (j) a nucleic acid molecule comprising a polynucleotide which is obtained by amplifying a cDNA library or a genomic library using the primers in Table III, application no. 20, column 8;
   [102813] and
   [102814] (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   [102815] or a nucleic acid molecule comprising a sequence which is complementary thereto;
      and, if desired
[102816] 2) nucleic acid molecule encoding a transit peptide, preferably encoding a plastidal transit peptide or a mitochondrial transit peptide;
   which are operable linked;]
   or comprising an expression cassette comprising
[102817] [0) a promoter, preferably a promoter selected from the group consisting of Big35S, PCUbi, Super and USP; and
[102818] 1) a nucleic acid molecule selected from the group consisting of
   [102819] (a) a nucleic acid molecule encoding a polypeptide shown in Table II, application no. 20, column 5 or 8, preferably shown in Table II A, application no. 20, column 5, or in Table II A, application no. 20, column 8, or in Table II B, application no. 20, column 8, or a homolog or a fragment thereof;
   [102820] (b) a nucleic acid molecule shown in Table I, application no. 20, column 5 or 8, preferably shown in Table I A, application no. 20, column 5, or in Table I A, application no. 20, column 8, or in Table I B, application no. 20, column 8, preferably the coding region thereof, or a homolog or a fragment thereof;
   [102821] (c) a nucleic acid molecule, which, as a result of the degeneracy of the genetic code, can be derived from a polypeptide sequence depicted in column 5 or 8 of Table II, application no. 20, preferably shown in Table II A, application no. 20, column 5, or in Table II A, application no. 20, column 8, or in Table II B, application no. 20, column 8;
   [102822] (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 20, preferably shown in Table I A, application no. 20, column 5, or in Table I A, application no. 20, column 8, or in Table I B, application no. 20, column 8, preferably the coding region thereof;
   [102823] (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   [102824] (f) a nucleic acid molecule which hybridizes with a nucleic acid molecule of (a), (b), (c), (d) or (e) under stringent hybridization conditions;
   [102825] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   [102826] (h) a nucleic acid molecule encoding a polypeptide comprising the consensus sequence or one or more polypeptide motifs as shown in column 8 of Table IV, application no. 20;
   [102827] (i) a nucleic acid molecule encoding a polypeptide having the activity represented by a respective protein as depicted in column 5 of Table II, application no. 20;
   [102828] (j) a nucleic acid molecule comprising a polynucleotide which is obtained by amplifying a cDNA library or a genomic library using the primers in Table III, application no. 20, column 8;
   [102829] and
   [102830] (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   [102831] or a nucleic acid molecule comprising a sequence which is complementary thereto;]
      and, if desired
[102832] 2) nucleic acid molecule encoding a transit peptide, preferably encoding a plastidal transit peptide or a mitochondrial transit peptide;
   which are operable linked;]
   or comprising or being transformed by a vector comprising
[102833] [1) a nucleic acid molecule selected from the group consisting of
   [102834] (a) a nucleic acid molecule encoding a polypeptide shown in Table II, application no. 20, column 5 or 8, preferably shown in Table II A, application no. 20, column 5, or in Table II A, application no. 20, column 8, or in Table II B, application no. 20, column 8, or a homolog or a fragment thereof;
   [102835] (b) a nucleic acid molecule shown in Table I, application no. 20, column 5 or 8, preferably shown in Table I A, application no. 20, column 5, or in Table I A, application no. 20, column 8, or in Table I B, application no. 20, column 8, preferably the coding region thereof, or a homolog or a fragment thereof;
   [102836] (c) a nucleic acid molecule, which, as a result of the degeneracy of the genetic code, can be derived from a polypeptide sequence depicted in column 5 or 8 of Table II, application no. 20, preferably shown in Table II A, application no. 20, column 5, or in Table II A, application no. 20, column 8, or in Table II B, application no. 20, column 8;
   [102837] (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 20, preferably shown in Table I A, application no. 20, column 5, or in Table I A, application no. 20, column 8, or in Table I B, application no. 20, column 8, preferably the coding region thereof;
   [102838] (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
   [102839] (f) a nucleic acid molecule which hybridizes with a nucleic acid molecule of (a), (b), (c), (d) or (e) under stringent hybridization conditions;
   [102840] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   [102841] (h) a nucleic acid molecule encoding a polypeptide comprising the consensus sequence or one or more polypeptide motifs as shown in column 8 of Table IV, application no. 20;
   [102842] (i) a nucleic acid molecule encoding a polypeptide having the activity represented by a respective protein as depicted in column 5 of Table II, application no. 20;
   [102843] (j) a nucleic acid molecule comprising a polynucleotide which is obtained by amplifying a cDNA library or a genomic library using the primers in Table III, application no. 20, column 8;
   [102844] and
   [102845] (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
   [102846] or a nucleic acid molecule comprising a sequence which is complementary thereto;]
      whereby, preferably, the nucleic acid molecule according to 1 (a), (b), (c), (d), (e), (f), (g), (h), (i), (j) or (k) distinguishes over the sequence depicted in Table I A and/or I B, application no. 20, column 5 or 8, or the coding regions thereof, by one or more nucleotides (especially but not exceeding 5%, preferably 4%, 3%, 2%, 1%, 0.5%, 0.2% or 0.1%). In an embodiment, the nucleic acid molecule of the invention does not consist of the sequence shown in Table I A and/or I B, application no. 20, column 5 or 8, or the coding region thereof. In another embodiment, the nucleic acid molecule of the present invention, preferably the coding region thereof, is at least 30%, 40%, 50%, 60%, 70%, 80%, 90%, 95% identical but less than 100%, 99.999%, 99.99%, 99.9%, 99%, 98%, 97%, 96% or 95% identical to the sequence shown in Table I A and/or I B, application no. 20, column 5 or 8, or the coding region thereof, respectively. In a further embodiment the nucleic acid molecule does not encode the polypeptide sequence shown in Table II A and/or II B, application no. 20, column 5 or 8 but homologs thereof. Accordingly, in one embodiment, the nucleic acid molecule of the present invention encodes in one embodiment a polypeptide which differs at least in one or more amino acids (especially but not exceeding 5%, preferably 4%, 3%, 2%, 1%, 0.5%, 0.2% or 0.1%) from the polypeptide shown in Table II, application no. 20, column 5 or 8 and does not encode a protein of the sequence shown in Table II A and/or II B, application no. 20, column 5 or 8. Accordingly, in one embodiment, the protein encoded by a sequence of a nucleic acid according to 1 (a), (b), (c), (d), (e), (f), (g), (h), (i), (j) or (k) does not consist of the sequence shown in Table I A and/or I B, application no. 20, column 5 or 8, or the coding region thereof. In a further embodiment, the protein of the present invention is at least 30%, 40%, 50%, 60%, 70%, 80%, 90%, 95% identical to protein sequence depicted in Table II A and/or II B, application no. 20, column 5 or 8 but less than 100%, preferably less than 99.999%, 99.99%, 99.9%, 99%, 98%, 97%, 96% or 95% identical to the sequence shown in Table II A and/or II B, application no. 20, column 5 or 8; as well as respective transgenic cells, tissue, parts of such non-human organism, e.g. plant cells, plant tissue, part of plants, like leaves, roots, stems, blossoms, seeds, fruits, pollen and the like.
      (All the references to Tables I to IV are references to the corresponding lines of Tables I to IV.)

[102847] In a preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 20.

[102848] In another preferred embodiment of said transgenic non-human organism said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[102849] In another preferred embodiment of said transgenic non-human organism said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 20 and confers a generation or an increase of the respective fine chemical in a non-human organism or a part thereof as compared to a corresponding wild-type non-human organism or part thereof.

[102850] In another embodiment of said transgenic non-human organism the expression cassette comprises 2) a nucleic acid molecule encoding a transit peptide, preferably encoding a plastidal transit peptide, especially in case in column 6 of Table I, application no. 20, in the same line as of the respective nucleic acid molecule according to 1) “plastidic” is depicted, or encoding a mitochondrial transit peptide, especially in case in column 6 of Table I, application no. 20, in the same line as of the respective nucleic acid molecule according to 1) “mitochondric” is depicted.

[102851] In another embodiment thereof the expression cassette does not comprise 2) a nucleic acid molecule encoding a transit peptide, especially in case in column 6 of Table I, application no. 20, in the same line as of the respective nucleic acid molecule according to 1) “non-targeted” is depicted.

[102852] In another embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked.

[102853] In a preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 20.

[102854] In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[102855] In another preferred embodiment of said transgenic non-human organism said vector comprises beneath said nucleic acid molecule according to 1 (a), (b), (c), (d), (e), (f), (g), (h), (i), (j) or (k), a promoter, preferably a promoter selected from the group consisting of Big35S, PCUbi, Super and USP, which are operable linked; and said nucleic acid molecule according to 1 (a), (b), (c), (d), (e), (f), (g), (h), (i), (j) or (k) encodes a polypeptide which has the activity of the respective polypeptide represented by a protein as depicted in the same line in column 5 of Table II, application no. 20, and confers a generation or an increase of the respective fine chemical in a non-human organism or a part thereof as compared to a corresponding wild-type non-human organism or part thereof.

[102856] In another embodiment of said transgenic non-human organism said vector comprises beneath said nucleic acid molecule according to 1 (a), (b), (c), (d), (e), (f), (g), (h), (i), (j) or (k), a nucleic acid molecule encoding a transit peptide, preferably encoding a plastidal transit peptide, especially in case in column 6 of Table I, application no. 20, in the same line as of the respective nucleic acid molecule according to 1) “plastidic” is depicted, or encoding a mitochondrial transit peptide, especially in case in column 6 of Table I, application no. 20, in the same line as of the respective nucleic acid molecule according to 1) “mitochondric” is depicted, which are operable linked.

[102857] In another embodiment of said transgenic non-human organism said vector does not comprise beneath said nucleic acid molecule according to 1 (a), (b), (c), (d), (e), (f), (g), (h), (i), (j) or (k), a nucleic acid molecule encoding a transit peptide in case in column 6 of Table I, application no. 20, in the same line as of the respective nucleic acid molecule according to 1) “non-targeted” is depicted, which are operable linked.

[102858] In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 20, in the same line as of the respective nucleic acid molecule according to 1) “plastidic” is depicted, or encoding a mitochondrial transit peptide, especially in case in column 6 of Table I, application no. 20, in the same line as of the respective nucleic acid molecule according to 1) “mitochondric” is depicted, which are operable linked.

[102859] In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 20, in the same line as of the respective nucleic acid molecule according to 1) “non-targeted” is depicted, which are operable linked.

[102860] In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 20, in the same line as of the respective nucleic acid molecule according to 1) “plastidic” is depicted, or encoding a mitochondrial transit peptide, especially in case in column 6 of Table I, application no. 20, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 20.

[102861] In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 20, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein comprising a polypeptide as depicted in the respective line in column 5 of Table II, application no. 20.

[102862] In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 20, in the same line as of the respective nucleic acid molecule according to 1) “plastidic” is depicted, or encoding a mitochondrial transit peptide, especially in case in column 6 of Table I, application no. 20, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[102863] In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 20, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) confers a generation or an increase of the respective fine chemical in a non-human organism or a part thereof as compared to a corresponding wild-type non-human organism or part thereof.

[102864] In another embodiment of said transgenic non-human organism said vector comprises beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, preferably encoding a plastidal transit peptide, especially in case in column 6 of Table I, application no. 20, in the same line as of the respective nucleic acid molecule according to 1) “plastidic” is depicted, or encoding a mitochondrial transit peptide, especially in case in column 6 of Table I, application no. 20, in the same line as of the respective nucleic acid molecule according to 1) “mitochondric” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 20 and confers a generation or an increase of the respective fine chemical in a non-human organism or a part thereof as compared to a corresponding wild-type non-human organism or part thereof.

[102865] In another embodiment of said transgenic non-human organism said vector does not comprise beneath said nucleic acid molecule according to 1 (a), (b), (c), (d), (e), (f), (g), (h), (i), (j) or (k), and a promoter, preferably a promoter selected from the group consisting of Big35S, PCUbi, Super and USP; a nucleic acid molecule encoding a transit peptide, especially in case in column 6 of Table I, application no. 20, in the same line as of the respective nucleic acid molecule according to 1) “non-targeted” is depicted, which are operable linked; and wherein said nucleic acid molecule according to 1 (a), (b), (c), (d), (e), (f), (g), (h), (i), (j) or (k) encodes a polypeptide which has the activity of the respective polypeptide represented by a protein as depicted in the respective line in column 5 of Table II application no. 20 and confers a generation or an increase of the respective fine chemical in a non-human organism or a part thereof as compared to a corresponding wild-type non-human organism or part thereof.

[102866] In a preferred embodiment of said transgenic non-human organism said nucleic acid molecule according to 1 (a), (b), (c), (d), (e), (f), (g), (h), (i), (j) or (k), said promoter and optionally said nucleic acid molecule encoding a transit peptide are encompassed in an expression cassette in said vector. Accordingly an embodiment the invention provides a non-human organism comprising or being transformed by a vector comprising an expression cassette comprising said nucleic acid molecule according to 1 (a), (b), (c), (d), (e), (f), (g), (h), (i), (j) or (k), said promoter and optionally said a nucleic acid molecule encoding a transit peptide, operable linked. Said expression cassette may encompass a terminator, too.

[102867] [0291.1.7.20] to [0299.1.7.20] for the disclosure of these paragraphs see [0291.1.7.7] to [0299.1.7.7] above

[102868] The term “biological active portion” or “biological activity” means a polypeptide as depicted in the respective line in Table II, application no. 20, column 3 or a portion of said polypeptide which still has at least 10% or 20%, preferably 30%, 40%, 50% or 60%, especially preferably 70%, 75%, 80%, 90% or 95% of the enzymatic or biological activity of the natural or starting enzyme or protein.

[102869] [0301.1.7.20] to [0304.1.7.20] for the disclosure of these paragraphs see [0301.1.7.7] to [0304.1.7.7] above

[102870] Synthetic oligonucleotide primers for the amplification, e.g. as shown in the respective line in Table III, application no. 20, column 8, by means of polymerase chain reaction can be generated on the basis of a sequence shown herein, for example the sequence shown in the respective line in Table I, application no. 20, columns 5 or 8, or the sequences derived from Table II, application no. 20, columns 5 or 8, respectively.

[102871] Moreover, it is possible to identify a conserved region of a protein from various organism by carrying out protein sequence alignments with the polypeptide encoded by the nucleic acid molecules of the present invention, in particular with the sequences encoded by the nucleic acid molecule shown in the respective line in column 5 or 8 of Table I, application no. 20, from which conserved regions, and in turn, degenerate primers can be derived. Conserved regions are those, which show a very little variation in the amino acid in one particular position of several homologs from different origin. The consensus sequence and polypeptide motifs shown in the respective line in column 8 of Table IV, application no. 20, are derived from such alignments of homologous proteins.

[102872] In an embodiment of the present invention, in the method of the present invention the activity of a polypeptide comprising or consisting of a consensus sequence or at least one polypeptide motif shown in the respective line in Table IV, application no. 20, column 8, is increased. In another embodiment thereof, the method of the present invention relates to a polypeptide comprising or consisting of a consensus sequence shown in the respective line in Table IV, application no. 20, column 8 or comprising at least one polypeptide motif shown in the respective line in Table IV, application no. 20, column 8, whereby not more than 20, 15, 10, 9, 8, 7, 6, 5 4, 3, 2 or 1, or 0 of the amino acid positions indicated can be replaced by any amino acid. In another embodiment thereof, the method of the present invention relates to a polypeptide comprising or consisting of a consensus sequence shown in the respective line in Table IV, application no. 20, column 8 or comprising at least one polypeptide motif shown in the respective line in Table IV, application no. 20, column 8, whereby not more than 15%, 10%, 5%, 4%, 3%, 2% or 1%, or 0% of the amino acid positions defined as distinct specific amino acids are/is replaced by another amino acid. In another embodiment thereof, the method of the present invention relates to a polypeptide comprising or consisting of a consensus sequence shown in the respective line in Table IV, application no. 20, column 8 or comprising at least one polypeptide motif shown in the respective line in Table IV, application no. 20, column 8, whereby not more than 20, 15, 10, 9, 8, 7, 6, 5, 4, 3, 2 or 1, or 0 amino acids are inserted into a consensus sequence or polypeptide motif.

[102873] In an embodiment of the present invention, in the method of the present invention the activity of a polypeptide comprising or consisting of a consensus sequence or at least one polypeptide motif shown in the respective line in Table IV, application no. 20, column 8, is increased, whereby said polypeptide distinguishes over the sequence depicted in Table II, application no. 20, columns 5 or 8 by one or more amino acids.

[102874] In one embodiment, said polypeptide of the invention distinguishes over the sequence shown in the respective line in Table II, application no. 20, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II B, application no. 20, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in Table II, application no. 20, columns 5 or 8.

[102875] [0309.1.7.20] to [0321.1.7.20] for the disclosure of these paragraphs see [0309.1.7.7] to [0321.1.7.7] above

[102876] Polypeptides having above-mentioned activity, i.e. conferring the production or the increased production of the fine chemical hentriacontane as compared to a corresponding, e.g. non-transformed, wild type non-human organism, like a microorganism or a plant cell, plant or part thereof, can be encoded by other DNA sequences which hybridize to the sequences shown in the respective line in Table I, application no. 20, columns 5 and 8, preferably the coding region thereof, at least under relaxed hybridization conditions and which encode the expression of polypeptides conferring the production or the increased production of the respective fine chemical hentriacontane as compared to a corresponding, e.g. non-transformed, wild type non-human organism, like a microorganism or a plant cell, plant or part thereof.

[102877] [0323.1.7.20] to [0329.1.7.20] for the disclosure of these paragraphs see [0323.1.7.7] to [0329.1.7.7] above

[102878] Further, the nucleic acid molecule of the invention comprises a nucleic acid molecule, which is a complement of one of the nucleotide sequences of above-mentioned nucleic acid molecules or a portion thereof. A nucleic acid molecule or its sequence which is complementary to one of the nucleotide molecules or sequences shown in the respective line in Table I, application no. 20, columns 5 or 8, preferably the coding region thereof, homologs or fragments thereof, is one which is sufficiently complementary to one of the nucleotide molecules or sequences shown in the respective line in Table I, application no. 20, columns 5 or 8, preferably the coding region thereof, homologs or fragments thereof, such that it can hybridize to one of the nucleotide sequences shown in Table I, application no. 20, columns 5 or 8, preferably the coding region thereof, homologs or fragments thereof, thereby forming a stable duplex. Preferably, the hybridization is performed under stringent hybridization conditions. However, a complement of one of the herein disclosed sequences is preferably a sequence complement thereto according to the base pairing of nucleic acid molecules well known to the skilled person. For example, the bases A and G undergo base pairing with the bases T and U or C, resp. and visa versa. Modifications of the bases can influence the base-pairing partner.

[102879] The nucleic acid molecule of the invention comprises a nucleotide sequence which is at least about 30%, 35%, 40%, 45%, 50%, 55%, 60%, 65%, 70%, 75%, 80%, 90%, 95%, 96%, 97%, 98%, 99%, 99.5% or more homologous to a nucleotide sequence shown in Table I, application no. 20, columns 5 or 8, preferably the coding region thereof, homologs or fragments thereof, and preferably has above-mentioned activity, in particular conferring the production or the increased production of the fine chemical hentriacontane, respectively, after increasing the activity or an activity of a gene as shown in the respective line in Table I or of a gene product, e.g. as shown in the respective line in Table II, application no. 20, column 5 or 8, by for example in one embodiment expression either in the cytosol or in an organelle such as a plastid or mitochondria or both, preferably in a plastid, or in another embodiment non-targeted or targeted.

[102880] The nucleic acid molecule of the invention comprises a nucleotide sequence or molecule which hybridizes, preferably hybridizes under stringent conditions as defined herein, to one of the nucleic acid molecule shown in the respective line in Table I, preferably Table IB, application no. 20, columns 5 or 8, preferably the coding region thereof, fragments or homologs thereof, and encodes a protein having above-mentioned activity, e.g. conferring the production or the increased production of the respective fine chemical hentriacontane as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted, and optionally, the activity thereof is selected from the group consisting of 3-deoxy-7-phosphoheptulonate synthase, acid shock protein, amino-acid acetyltransferase, assembly protein for periplasmic nitrate reductase, At2g45420-protein, At5g43630-protein, ATP-dependent RNA helicase, b0833-protein, b1259-protein, b1445-protein, b1670-protein, b1930-protein, b2399-protein, b2653-protein, b3644-protein, cell division protein, cyclin D, cysteine synthase A, dihydroxyacetone kinase, electron transport complex protein, ethanolamine utilization protein, exonuclease subunit, fumarylacetoacetate hydrolase, GDP-mannose 4,6-dehydratase, inner membrane protein, ion-transport protein, L-asparaginase, MADS box protein transcription factor, mechanosensitive ion channel, metal-dependent hydrolase, methionine permease, O-antigen chain length determinant, ornithine decarboxylase, orotidine-5′-phosphate decarboxylase, peptide chain release factor, phospho-2-dehydro-3-deoxyheptonate aldolase, phosphoadenosine phosphosulfate reductase, phosphoglycolate phosphatase, pyrimidine-specific ribonucleoside hydrolase, pyruvate kinase, RNA-binding protein, S-adenosyl-L-methionine-dependent methyltransferase, serine protease, short-chain alcohol dehydrogenase family, sigma-54 dependent response regulator, spermidine synthase, sugar transport system permease protein, transcription factor, transcriptional regulator, YFL019C-protein, YHL039W-protein, YHR022C-A-protein, YJL127W-A-protein, ylr157c-protein, ynl321w-protein, ypr170c-protein, and YPR172W-protein, respectively.

[102881] Moreover, the nucleic acid molecule of the invention can comprise only a portion of the coding region of one of the nucleic acid molecules depicted in Table I, application no. 20, columns 5 or 8, for example a fragment which can be used as a probe or primer or a fragment encoding a biologically active portion of the polypeptide of the present invention or of a polypeptide used in the process of the present invention, i.e. having above-mentioned activity, e.g. conferring the production or the increased production of the respective fine chemical hentriacontane as compared to a corresponding, e.g. non-transformed, wild type non-human organism, like a microorganism or a plant cell, plant or part thereof, by for example in one embodiment expression either in the cytosol or in an organelle such as a plastid or mitochondria or both, preferably in a plastid, or in another embodiment non-targeted or targeted. The nucleotide sequences determined from the cloning of the present protein-according-to-the-invention-encoding gene allows for the generation of probes and primers designed for use in identifying and/or cloning its homologues from other cell types and organisms. The probe/primer typically comprises a substantially purified oligonucleotide. The oligonucleotide typically comprises a region of nucleotide sequence that hybridizes under stringent conditions to at least about 12, 15 preferably about 20 or 25, more preferably about 40, 50 or 75 consecutive nucleotides of a sense strand of one of the sequences set forth, e.g., in the respective line in Table I, application no. 20, column 5 or 8, preferably the coding region thereof, or a homolog or a fragment thereof, an anti-sense sequence of one of the sequences, e.g., set forth in the respective line in Table I, application no. 20, columns 5 or 8, preferably the coding region thereof, or a homolog or a fragment thereof or naturally occurring mutants thereof. Primers based on a nucleic acid molecule of invention can be used in PCR reactions to clone homologs of the polypeptide of the invention or of the polypeptide used in the process of the invention, e.g. as the primers described in the examples of the present invention, e.g. as shown in the examples. A PCR with the primers shown in the respective line in Table III, column 8 will result in a fragment of the gene product as shown in Table II, application no. 20, column 5.

[102882] Primer sets are interchangeable. The person skilled in the art knows how to design and combine said primers to result in the desired product, e.g. in a full length clone or a partial sequence. Probes based on the sequences of the nucleic acid molecule of the invention or used in the process of the present invention can be used to detect transcripts or genomic sequences encoding the same or homologous proteins. The probe can further comprise a label group attached thereto, e.g. the label group can be a radioisotope, a fluorescent compound, an enzyme, or an enzyme co-factor. Such probes can be used as a part of a genomic marker test kit for identifying cells which express a polypeptide of the invention or used in the process of the present invention, such as by measuring a level of an encoding nucleic acid molecule in a sample of cells, e.g. detecting mRNA levels or determining, whether a genomic gene comprising the sequence of the polynucleotide of the invention or used in the processes of the present invention has been mutated or deleted.

[102883] As the expression of a respective nucleic acids of the invention is related to the synthesis of the respective fine chemical hentriacontane its function as a probe extends to the detection of microorganisms, plant tissues, plants, plant variets, plant ecotypes or plant genera with varying capability or potential for synthesis of the respective fine chemical hentriacontane. Therefore in one embodiment the present invention relates to a method for analyzing the capability or potential of a plant tissue, a plant, a plant variety or plant ecotype to produce the fine chemical hentriacontane by using the nucleic acid of the invention or parts thereof as a probe to detect the amount of the nucleic acid of the invention in the non-human organism or a part thereof in comparison to another non-human organism.

[102884] The nucleic acid molecule of the invention encodes a polypeptide or portion thereof which includes an amino acid sequence which is sufficiently homologous to the amino acid sequence shown in respective line in Table II, application no. 20, columns 5 or 8 such that the protein or portion thereof maintains the ability to confer the production or the increased production of the respective fine chemical hentriacontane as compared to a corresponding, e.g. non-transformed, wild type non-human organism, like a microorganism or a plant cell, plant or part thereof, in particular increasing the activity as mentioned above or as described in the examples in microorganisms or plants.

[102885] As used herein, the language “sufficiently homologous” refers to proteins or portions thereof which have amino acid sequences which include a minimum number of identical or equivalent amino acids (e.g. an amino acid which has a similar side chain as an amino acid in one of the sequences of the polypeptide of the present invention) to an amino acid sequence shown in the respective line in Table II, application no. 20, columns 5 or 8 such that the protein or portion thereof is able to confer the production or the increased production of the respective fine chemical hentriacontane as compared to a corresponding, e.g. non-transformed, wild type non-human organism, like a microorganism or a plant cell, plant or part thereof. For example having the activity of a protein as shown in the respective line in Table II, application no. 20, column 3 and as described herein.

[102886] In an embodiment of the present invention, the nucleic acid molecule of the present invention comprises a nucleic acid molecule that encodes a portion of the protein of the present invention. The protein is at least about 30%, 35%, 40%, 45% 50%, 55%, 60%, 65%, 70%, 75%, 80%, 85%, 90%, 91%, 92%, 93%, 94%, 95%, 96%, 97%, 98%, 99%, 99.5% or more homologous to an entire amino acid sequence shown in the respective line of Table II, application no. 20, column 5 or 8 and has the above-mentioned activity, e.g. conferring the production or the increased production of the respective fine chemical hentriacontane as compared to a corresponding, e.g. non-transformed, wild type non-human organism, like a microorganism or a plant cell, plant or part thereof by, for example in a embodiment expression either in the cytosol or in an organelle such as a plastid or mitochondria or both, preferably in a plastid, or in another embodiment by targeted or non-targeted expression.

[102887] for the disclosure of this paragraph see [0338.1.7.7] above

[102888] for the disclosure of this paragraph see [0339.1.7.7] above

[102889] The invention further relates to nucleic acid molecules or methods using said nucleic acid molecules that differ from one of the nucleotide sequences shown in the respective line in Table I, application no. 20, columns 5 or 8, preferably the coding region thereof, or fragments or homologs thereof, due to degeneracy of the genetic code but encode a polypeptide of the present invention, in particular a polypeptide having the above-mentioned activity, e.g. as those polypeptides depicted in the respective line in Table II, application no. 20, columns 5 or 8, fragments or homologs thereof. Advantageously, the nucleic acid molecule of the invention comprises, or in an other embodiment has, a nucleotide sequence encoding a protein comprising, or in an other embodiment having, an amino acid sequence shown in the respective line in Table II, application no. 20, columns 5 or 8, fragments or homologs thereof. In a further embodiment, the nucleic acid molecule of the invention encodes a full length protein which is substantially homologous to an amino acid sequence shown in the respective line in Table II, application no. 20, columns 5 or 8, or homologs thereof. However, in an embodiment, the nucleic acid molecule of the present invention does not consist of the sequence shown in the respective line in Table I, preferably Table IA, application no. 20, columns 5 or 8, preferably the coding region thereof, but the functional homologs thereof.

[102890] [0341.1.7.20] to [0343.1.7.20] for the disclosure of these paragraphs see [0341.1.7.7] to [0343.1.7.7] above

[102891] Accordingly, in another embodiment, a nucleic acid molecule of the invention is at least 15, 20, 25 or 30 nucleotides in length. Preferably, it hybridizes under stringent conditions to a nucleic acid molecule comprising a nucleotide sequence of the nucleic acid molecule of the present invention or used in the process of the present invention, e.g. comprising the sequence shown in the respective line in Table I, application no. 20, columns 5 or 8, preferably the coding region thereof. In another embodiment the nucleic acid molecule is preferably at least 20, 30, 50, 100, 250 or more nucleotides in length.

[102892] for the disclosure of this paragraph see [0345.1.7.7] above

[102893] Preferably, a nucleic acid molecule of the invention that hybridizes under stringent conditions to a sequence shown in the respective line in Table I, application no. 20, columns 5 or 8, preferably the coding region thereof, corresponds to a naturally-occurring nucleic acid molecule of the invention. As used herein, a “naturally-occurring” nucleic acid molecule refers to a RNA or DNA molecule having a nucleotide sequence that occurs in nature (e.g. encodes a natural protein). Preferably, the nucleic acid molecule encodes a natural protein having above-mentioned activity, e.g. conferring the production or the increased production of the respective fine chemical hentriacontane as compared to a corresponding, e.g. non-transformed, wild type non-human organism, like a microorganism or a plant cell, plant or part thereof after increasing the expression or activity thereof or the activity of a protein of the invention or used in the process of the invention, in an embodiment for example expression either in the cytosol or in an organelle such as a plastid or mitochondria, preferably in plastids, or, in another embodiment by targeted or non-targeted expression.

[102894] for the disclosure of this paragraph see [0347.1.7.7] above

[102895] For example, nucleotide substitutions leading to amino acid substitutions at “non-essential” amino acid residues can be made in a sequence of the nucleic acid molecule of the invention or used in the process of the invention, e.g. shown in the respective line in Table I, application no. 20, columns 5 or 8, preferably the coding region thereof, or fragments or homologs thereof.

[102896] for the disclosure of this paragraph see [0349.1.7.7] above

[102897] for the disclosure of this paragraph see [0350.1.7.7] above

[102898] Accordingly, the invention relates to nucleic acid molecules encoding a polypeptide having above-mentioned activity, in a non-human organisms or parts thereof by for example expression either in the cytosol or in an organelle such as a plastid or mitochondria or both, preferably in plastids, that contain changes in amino acid residues that are not essential for said activity. Such polypeptides differ in amino acid sequence from a sequence depicted in the respective line in Table II, especially Table IIA, application no. 20, columns 5 or 8, but retain said activity described herein. The nucleic acid molecule can comprise a nucleotide sequence encoding a polypeptide, wherein the polypeptide comprises an amino acid sequence at least about 50% identical to an amino acid sequence shown in the respective line in Table II, application no. 20, columns 5 or 8, and is capable of conferring the production or the increased production of the respective fine chemical hentriacontane as compared to a corresponding, e.g. non-transformed, wild type non-human organism, like a microorganism or a plant cell, plant or part thereof, after increasing its activity for example in an embodiment by expression either in the cytosol or in an organelle such as a plastid or mitochondria or both, preferably in plastids, or, in another embodiment by targeted or non-targeted expression. Preferably, the protein encoded by the nucleic acid molecule is at least about 60%, 70%, 80%, 90%, 95%, 96%, 97%, 98%, 99% or 99.5% identical to the sequence shown in the respective line in Table II, application no. 20, columns 5 or 8.

[102899] [0352.1.7.20] to [0357.1.7.20] for the disclosure of these paragraphs see [0352.1.7.7] to [0357.1.7.7] above

[102900] Functional equivalents of the nucleic acid molecules according to the present invention and/or used in the process according to the present invention represent an embodiment of homologs. Functional equivalents derived from the nucleic acid molecules as shown in the respective line in Table I, application no. 20, columns 5 or 8, preferably the coding region thereof, according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the nucleic acid molecules as shown in the respective line in Table I, application no. 20, columns 5 or 8, preferably the coding region thereof, according to the invention and encode polypeptides having essentially the same properties as the polypeptide as shown in the respective line in Table II, application no. 20, columns 5 or 8.

[102901] Functional equivalents derived from one of the polypeptides as shown in the respective line in Table II, application no. 20, columns 5 or 8 according to the invention by substitution, insertion or deletion have at least 30%, 35%, 40%, 45%, 50%, 55%, 60%, 65%, 70%, 75%, 80%, 85%, 90%, 91%, 92%, 93%, 94%, 95%, 96%, 97%, 98%, 99% or 99.5% homology with one of the polypeptides as shown in the respective line in Table II, application no. 20, columns 5 or 8 according to the invention and having essentially the same properties as the polypeptide as shown in the respective line in Table II, application no. 20, columns 5 or 8.

[102902] for the disclosure of this paragraph see [0359.1.7.7] above

[102903] A nucleic acid molecule encoding a homolog to a polypeptide depicted in the respective line in Table II, application no. 20, columns 5 or 8 can be created by introducing one or more nucleotide substitutions, additions or deletions into the nucleic acid molecule of the present invention, in particular of Table I, application no. 20, columns 5 or 8, in the respective line, such that one or more amino acid substitutions, additions or deletions are introduced into the encoded protein. Mutations can be introduced into the nucleic acid molecules as depicted in the respective line in Table I, application no. 20, columns 5 or 8, preferably the coding region thereof, by standard techniques, such as site-directed mutagenesis, PCR-mediated mutagenesis or other methods known to the person skilled in the art.

[102904] [0361.1.7.20] to [0363.1.7.20] for the disclosure of these paragraphs see [0361.1.7.7] to [0363.1.7.7] above

[102905] Homologues of the nucleic acid sequences used, with the sequences shown in the respective line in Table I, application no. 20, columns 5 or 8, preferably the coding region thereof, comprise also allelic variants with at least approximately 30%, 35%, 40%, 45%, 50%, 55%, 60%, 65%, 70%, 75%, 80%, 85%, 90%, 91%, 92%, 93%, 94% 95%. 96%, 97%, 98%, 99%, 99.5% or more % homology with one of the nucleotide sequences shown or the above-mentioned derived nucleic acid sequences or their homologues or derivatives or parts of these. Allelic variants encompass in particular functional variants which can be obtained by deletion, insertion or substitution of nucleotides from the sequences shown in the respective line, preferably in Table I, columns 5 or 8, preferably the coding region thereof, or from the derived nucleic acid sequences, the intention being, however, that the enzymatic activity or the biological activity of the resulting proteins synthesized is advantageously retained or increased.

[102906] In one embodiment of the present invention, the nucleic acid molecule of the invention or used in the process of the invention comprises the sequences shown in the respective line in any of the Table I, application no. 20, columns 5 or 8, preferably the coding region thereof, fragments or homologs thereof. It is preferred that the nucleic acid molecule comprises as little as possible other nucleotides not shown in the respective line in any one of Table I, application no. 20, columns 5 or 8, preferably the coding region thereof, fragments or homologs thereof. In one embodiment, the nucleic acid molecule comprises less than 500, 400, 300, 200, 100, 90, 80, 70, 60, 50 or 40 further nucleotides. In a further embodiment, the nucleic acid molecule comprises less than 30, 20 or 10 further nucleotides. In one embodiment, the nucleic acid molecule used in the process of the invention is identical to the sequences shown in the respective line in Table I, application no. 20, columns 5 or 8, preferably the coding region thereof.

[102907] Also preferred is that the nucleic acid molecule used in the process of the invention encodes a polypeptide comprising the sequence shown in the respective line in Table II, application no. 20, columns 5 or 8, fragments or homologs thereof. In one embodiment, the nucleic acid molecule encodes less than 150, 130, 100, 80, 60, 50, 40 or 30 further amino acids. In a further embodiment, the encoded polypeptide comprises less than 20, 15, 10, 9, 8, 7, 6 or 5 further amino acids. In one embodiment used in the inventive process, the encoded polypeptide is identical to the sequences shown in the respective line in Table II, application no. 20, columns 5 or 8.

[102908] In one embodiment, the nucleic acid molecule of the invention or used in the process encodes a polypeptide comprising the sequence shown in the respective line in Table II, application no. 20, columns 5 or 8, or fragments or homologs thereof, and comprises less than 100 further nucleotides. In a further embodiment, said nucleic acid molecule comprises less than 30 further nucleotides. In one embodiment, the nucleic acid molecule used in the process is identical to a coding sequence of the sequences shown in the respective line in Table I, application no. 20, columns 5 or 8.

[102909] Polypeptides (=proteins), which still have the essential biological or enzymatic activity of the polypeptide of the present invention conferring the production or the increased production of the respective fine chemical hentriacontane as compared to a corresponding, e.g. non-transformed, wild type non-human organism, like a microorganism or a plant cell, plant or part thereof, i.e. whose activity is essentially not reduced, are polypeptides with at least 10%, 20%, 30%, 40%, 50%, 60%, 70%, 80%, 90%, 95% or more of the wild type biological activity or enzymatic activity, advantageously, the activity is essentially not reduced in comparison with the activity of a polypeptide shown in the respective line in Table II, application no. 20, columns 5 or 8 expressed under identical conditions.

[102910] Homologues of nucleic acid molecules shown in the respective line in Table I, application no. 20, columns 5 or 8, preferably the coding region thereof, or fragments thereof, or of the derived sequences shown in the respective line in Table II, application no. 20, columns 5 or 8, or fragments thereof, also mean truncated sequences, cDNA, single-stranded DNA or RNA of the coding and noncoding DNA sequence. Homologues of said sequences are also understood as meaning derivatives, which comprise noncoding regions such as, for example, UTRs, introns, terminators, enhancers or promoter variants. The promoters upstream of the nucleotide sequences stated can be modified by one or more nucleotide substitution(s), insertion(s) and/or deletion(s) without, however, interfering with the functionality or activity either of the promoters, the open reading frame (=ORF) or with the 3′-regulatory region such as terminators or other 3′-regulatory regions, which are far away from the ORF. It is furthermore possible that the activity of the promoters is increased by modification of their sequence, or that they are replaced completely by more active promoters, even promoters from heterologous organisms.

[102911] Appropriate promoters are known to the person skilled in the art and are mentioned herein below.

[102912] [0370.1.7.20] to [0379.1.7.20] for the disclosure of these paragraphs see [0370.1.7.7] to [0379.1.7.7] above

[102913] Moreover, a native polypeptide conferring the increase of the respective fine chemical hentriacontane in a non-human organism or a part thereof can be isolated from cells (e.g., endothelial cells), for example using the antibody of the present invention as described below, in particular, an antibody against proteins having 3-deoxy-7-phosphoheptulonate synthase, acid shock protein, amino-acid acetyltransferase, assembly protein for periplasmic nitrate reductase, At2g45420-protein, At5g43630-protein, ATP-dependent RNA helicase, b0833-protein, b1259-protein, b1445-protein, b1670-protein, b1930-protein, b2399-protein, b2653-protein, b3644-protein, cell division protein, cyclin D, cysteine synthase A, dihydroxyacetone kinase, electron transport complex protein, ethanolamine utilization protein, exonuclease subunit, fumarylacetoacetate hydrolase, GDP-mannose 4,6-dehydratase, inner membrane protein, ion-transport protein, L-asparaginase, MADS box protein transcription factor, mechanosensitive ion channel, metal-dependent hydrolase, methionine permease, O-antigen chain length determinant, ornithine decarboxylase, orotidine-5′-phosphate decarboxylase, peptide chain release factor, phospho-2-dehydro-3-deoxyheptonate aldolase, phosphoadenosine phosphosulfate reductase, phosphoglycolate phosphatase, pyrimidine-specific ribonucleoside hydrolase, pyruvate kinase, RNA-binding protein, S-adenosyl-L-methionine-dependent methyltransferase, serine protease, short-chain alcohol dehydrogenase family, sigma-54 dependent response regulator, spermidine synthase, sugar transport system permease protein, transcription factor, transcriptional regulator, YFL0190-protein, YHL039W-protein, YHR022C-A-protein, YJL127W-A-protein, ylr157c-protein, ynl321w-protein, ypr170c-protein, or YPR172W-protein activity, respectively, or an antibody against polypeptides as shown in the respective line in Table II, application no. 20, columns 5 or 8, or fragments or homologs thereof which can be produced by standard techniques utilizing the polypeptid of the present invention or fragment thereof, i.e., the polypeptide of this invention (FCRP). Preferred are monoclonal antibodies.

[102914] for the disclosure of this paragraph see [0381.1.7.7] above

[102915] In one embodiment, the present invention relates to a polypeptide as depicted in the respective line in Table II, especially in Table IIA or especially in Table IIB, application no. 20, columns 5 or 8, or fragment or homolog thereof or as coded by the nucleic acid molecule shown in the respective line in Table I, especially Table IA or especially Table IB, application no. 20, columns 5 or 8, preferably the coding region thereof, or fragment or homolog thereof.

[102916] In an embodiment, the present invention relates to a polypeptide comprising or consisting of a consensus sequence shown in the respective line in Table IV, application no. 20, column 8 or at least one polypeptide motif shown in the respective line in Table IV, application no. 20, column 8.

[102917] In an embodiment thereof, the present invention relates to a polypeptide comprising or consisting of a consensus sequence shown in the respective line in Table IV, application no. 20, column 8 or comprising at least one polypeptide motif shown in the respective line in Table IV, application no. 20, column 8, whereby not more than 20, 15, 10, 9, 8, 7, 6, 5, 4, 3, 2, or 1, or 0 of the amino acids can be replaced by any amino acid.

[102918] In another embodiment thereof, the present invention relates to a polypeptide comprising or consisting of a consensus sequence shown in the respective line in Table IV, application no. 20, column 8 or comprising at least one polypeptide motif shown in the respective line in Table IV, application no. 20, column 8, whereby not more than 15%, 10%, 5%, 4%, 3%, 2% or1% or 0% of the amino acids defined as distinct specific amino acids are/is replaced by another amino acid.

[102919] In another embodiment thereof, the present invention relates to a polypeptide comprising or consisting of a consensus sequence shown in the respective line in Table IV, application no. 20, column 8 or comprising at least one polypeptide motif shown in the respective line in Table IV, application no. 20, column 8, whereby not more than 20, 15, 10, 9, 8, 7, 6, 5, 4, 3, 2, or 1, or 0 amino acids are inserted into the consensus sequence or polypeptide motif.

[102920] In an embodiment of the present invention, the polypeptide of the present invention comprises or consists of a consensus sequence or at least one polypeptide motif shown in the respective line in Table IV, application no. 20, column 8, whereby said polypeptide distinguishes over the sequence depicted in the respective line in Table II, application no. 20, columns 5 or 8 by one or more amino acids. In one embodiment, said polypeptide of the invention distinguishes over the sequence shown in the respective line in Table II, application no. 20, columns 5 and 8 by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 20, columns 5 and 8 by not more than 80%, 70%, 60%, 50%, 40%, 30%, 20%, 10%, 5%, 4%, 3%, 2%, 1% or 0.5%. In another embodiment, said polypeptide of the invention does not consist of the sequence shown in the respective line in Table II, application no. 20, columns 5 and 8.

[102921] In an embodiment, the invention relates to a polypeptide conferring an increase in the respective fine chemical hentriacontane in a non-human organism, especially a microorganism or a plant, or a part thereof, being encoded by the nucleic acid molecule of the invention or used in the process of the invention and having a sequence which distinguishes over the sequence as shown in the respective line in Table II, application no. 20, columns 5 or 8 by one or more amino acids (but not exceeding 5%, preferably 4%, 3%, 2%, 1%, 0.5%, 0.2% or 0.1%). In an embodiment, said polypeptide of the invention does not comprise or consist of the sequence shown in the respective line in Table II, application no. 20, columns 5 or 8. In an embodiment, said polypeptide of the present invention is less than 100%, 99.999%, 99.99%, 99.9% or 99% identical. In one embodiment, said polypeptide which differs at least in one or more amino acids (but not exceeding 5%, preferably 4%, 3%, 2%, 1%, 0.5%, 0.2% or 0.1%) from the polypeptide shown in the respective line in Table II, application no. 20, columns 5 and 8 does not comprise a protein of the sequence shown in the respective line in Table II A and/or II B, application no. 20, columns 5 or 8.

[102922] In an embodiment, the present invention relates to a polypeptide having the activity of the protein as shown in the respective line in Table II, application no. 20, column 3, which distinguishes over the sequence depicted in the respective line in Table II, application no. 20, columns 5 or 8 by one or more amino acids, preferably by more than 5, 6, 7, 8 or 9 amino acids, preferably by more than 10, 15, 20, 25 or 30 amino acids, even more preferred are more than 40, 50, or 60 amino acids and, preferably, the sequence of the polypeptide of the invention distinguishes from the sequence shown in the respective line in Table II, application no. 20, columns 5 and 8 by not more than 80%, 70%, 60%, 50%, 40%, 30%, 20%, 10%, 5%, 4%, 3%, 2%, 1% or 0.5%.

[102923] In another embodiment the polypeptide of the invention takes the form of a preprotein consisting of a plastidial or mitochondrial transit peptide joint to a polypeptide having the activity of the protein as shown in Table II, column 3, from which the transit peptide is preferably cleaved off upon transport of the preprotein into the organelle for example into the plastid or mitochondrion. In another embodiment the polypeptide of the invention takes not the form of a preprotein.

[102924] for the disclosure of this paragraph see [0390.1.7.7] above

[102925] for the disclosure of this paragraph see [0391.1.7.7] above

[102926] A polypeptide of the invention can participate in the process of the present invention. The polypeptide or a portion thereof comprises preferably an amino acid sequence, which is sufficiently homologous to an amino acid sequence shown in the respective line in Table II A and/or II B, application no. 20, columns 5 or 8.

[102927] Further, the polypeptide can have an amino acid sequence which is encoded by a nucleotide sequence which hybridizes, preferably hybridizes under stringent conditions as described above, to a nucleotide sequence of the nucleic acid molecule of the present invention. Accordingly, the polypeptide has an amino acid sequence which is encoded by a nucleotide sequence that is at least about 35%, 40%, 45%, 50%, 55%, 60%, 65%, 70%, 75%, 80%, 85%, 90%, 91%, 92%, 93%, 94%, 95%, 96%, 97%, 98%, 99%, 99.5% or more homologous to one of the amino acid sequences as shown in the respective line in Table II A and/or II B, application no. 20, columns 5 or 8, or fragments thereof. The preferred polypeptide of the present invention preferably possesses at least one of the activities according to the invention and described herein. A preferred polypeptide of the present invention includes an amino acid sequence encoded by a nucleotide sequence which hybridizes, preferably hybridizes under stringent conditions, to a nucleotide sequence shown in the respective line in Table I A and/or I B, application no. 20, columns 5 or 8, preferably the coding region thereof, or fragments thereto or which is homologous thereto, as defined above

[102928] The polypeptide of the present invention can vary from the sequences shown in the respective line in Table II A and/or II B, application no. 20, columns 5 or 8 in amino acid sequence due to natural variation or mutagenesis, as described in detail herein. Accordingly, the polypeptide comprise an amino acid sequence which is at least about 35%, 40%, 45%, 50%, 55%, 60%, 65%, 70%, 75%, 80%, 85% or 90, 91%, 92%, 93%, 94%, 95%, 96%, 97%, 98%, 99%, 99.5% or more homologous to an entire amino acid sequence shown in the respective line in Table II A and/or II B, application no. 20, columns 5 or 8.

[102929] Biologically active portions of a polypeptide of the present invention include peptides comprising amino acid sequences derived from the amino acid sequence of the polypeptide of the present invention or used in the process of the present invention, e.g., the amino acid sequence shown in the respective line in Table II, application no. 20, columns 5 or 8 or the amino acid sequence of a protein homologous thereto, which include fewer amino acids than a full length polypeptide of the present invention or used in the process of the present invention or the full length protein which is homologous to a polypeptide of the present invention or used in the process of the present invention depicted herein, and exhibit at least one activity of a polypeptide of the present invention or used in the process of the present invention.

[102930] for the disclosure of this paragraph see [0396.1.7.7] above

[102931] Manipulation of the nucleic acid molecule of the invention may result in the production of a protein having differences from the protein as shown in the respective line in Table II, application no. 20, column 5 or 8. Differences shall mean at least one amino acid different from the sequences as shown in the respective line in Table II, application no. 20, column 5 or 8, preferably at least 2, 3, 4, 5, 6, 7, 8, 9 or 10 amino acids (especially but not exceeding 0.1%, preferably 0.2%, 0.5%, 1%, 2%, 3% 4% or 5%), more preferably at least 15, 20, 25, 30, 35, 40, 45 or 50 amino acids (especially but not exceeding 0.5%, preferably 1%, 2%, 3% 4%, 5%, 10% or 25%) different from the sequences as shown in the respective line in Table II, application no. 20, column 5 or 8. These proteins may be improved in efficiency or activity, or may be more stable and therefore present in greater numbers in the cell as compared to the wild-type cell.

[102932] Any mutagenesis strategies for the polypeptide of the present invention or the polypeptide used in the process of the present invention to result in increasing said activity are not meant to be limiting; variations on these strategies will be readily apparent to one skilled in the art. Using such strategies, and incorporating the mechanisms disclosed herein, the nucleic acid molecule and polypeptide of the invention may be utilized to generate algae, ciliates, fungi, other microorganisms like C. glutamicum or plants or parts thereof, expressing proteins as shown in the respective line in Table II, application no. 20, column 5 or 8, or mutated proteins thereof. The nucleic acid molecules and polypeptide molecules of the invention are expressed such that the yield, production, and/or efficiency of production of a desired compound is improved.

[102933] [0399.1.7.20] to [0401.1.7.20] for the disclosure of these paragraphs see [0399.1.7.7] to [0401.1.7.7] above

[102934] In one embodiment, a protein (=polypeptide) as shown in the respective line in Table II, application no. 20, column 5 or 8 refers to a polypeptide having an amino acid sequence corresponding to the polypeptide of the invention, especially to a polypeptide as shown in Table II, application no. 20, columns 5 or 8 or homologs thereof, or used in the process of the invention, whereas a “non- inventive protein or polypeptide” or “other polypeptide” refers to a polypeptide having an amino acid sequence corresponding to a protein which is not substantially homologous to a polypeptide of the invention, preferably which is not substantially homologous to a polypeptide or protein as shown in the respective line in Table II, application no. 20, column 5 or 8 or homologs thereof, e.g., a protein which does not confer the activity described herein and which is derived from the same or a different organism.

[102935] [0403.1.7.20] to [0409.1.7.20] for the disclosure of these paragraphs see [0403.1.7.7] to [0409.1.7.7] above

[102936] As the nucleic acid molecules of the invention and/or the expression thereof is related to the synthesis of the respective fine chemical hentriacontane its function as a probe extends to the detection of microorganisms, plant tissues, plants, plant variets, plant ecotypes or plant genera with varying, advantageously increased, capability or potential for synthesis of the respective fine chemical hentriacontane. Therefore in one embodiment the present invention relates to a method for analyzing the capability or potential of a plant tissue, a plant, a plant variety or ecotype to produce the respective fine chemical hentriacontane by using the respective antibody of the invention as a probe to detect the amount of the polypeptide encoded by said nucleic acid molecule of the invention in a non-human organism in comparison to another organism.

[102937] Therefore in one embodiment the invention relates to a method to probe a non-human organism for its capability for the production of the respective fine chemical comprising the steps of
[102938] a) analyzing the sequence and/or the expression of the respective nucleic acid molecule of the invention in least two non-human organisms which differ in the capability to produce the respective fine chemical;
[102939] b) associate the different sequence and/or expression level of the nucleic acid molecules of the invention with their different capability to produce the respective fine chemical;
[102940] c) analyze the sequence and/or the expression of the nucleic acid molecule of the invention in at least a third non-human organism;
[102941] d) predict the capability of the at least third organism to produce the respective fine chemical based by comparing the sequence and/or expression of the nucleic acid of the invention to the sequences and/or expression levels of the nucleic acid molecule of the invention of the at least two organisms analyzed in step a.

[102942] [0411.1.7.20] to [0430.1.7.20] for the disclosure of these paragraphs see [0411.1.7.7] to [0430.1.7.7] above

[102943] Accordingly the present invention relates to any cell transgenic for any nucleic acid characterized as part of the invention, e.g. conferring the increase of the respective fine chemical hentriacontane in a cell or a non-human organism or a part thereof, e.g. the nucleic acid molecule of the invention, the nucleic acid construct of the invention, the vector of the invention, the expression cassette according to the invention, or a nucleic acid molecule encoding the polypeptide of the invention, e.g. encoding a polypeptide having an activity as the protein as shown in the respective line in Table II, application no. 20, column 3. Due to the above-mentioned activity the respective fine chemical hentriacontane content in a cell or a non-human organism is increased. For example, due to modulation or manipulation, the cellular activity is increased, in a preferred embodiment in organelles such as plastids or mitochondria, e.g. due to an increased expression or specific activity or specific targeting of the subject matters of the invention in a cell or a non-human organism or a part thereof especially in organelles such as plastids or mitochondria, or in another embodiment in the cytosol. Transgenic for a polypeptide having a protein or a protein activity means herein that due to modulation or manipulation of the genome, the activity of protein as shown in the respective line in Table II, application no. 20, column 3 or a protein as shown in the respective line in Table II, application no. 20, column alike activity is increased in the cell or non-human organism or part thereof, especially in organelles such as plastids or mitochondria, or especially in the cytosol. Examples are described above in context with the process of the invention.

[102944] for the disclosure of this paragraph see [0432.1.7.7] above

[102945] A naturally occurring expression cassette—for example the naturally occurring combination of the promoter of the gene encoding a protein as shown in the respective line in Table II, application no. 20, column 5 or 8, preferably the coding region thereof, or fragments or homologs thereof with the corresponding encoding gene—becomes a transgenic expression cassette when it is modified by non-natural, synthetic “artificial” methods such as, for example, mutagenesis. Such methods have been described (U.S. Pat. No. 5,565,350; WO 00/15815; also see above).

[102946] for the disclosure of this paragraph see [0434.1.7.7] above

[102947] for the disclosure of this paragraph see [0435.1.7.7] above

[102948] Transgenic plants comprising the respective fine chemical(s) synthesized in the process according to the invention can be marketed directly without isolation of the fine chemical synthesized. In the process according to the invention, plants are understood as meaning all plant parts, plant organs such as leaf, stalk, root, tubers or seeds or propagation material or harvested material or the intact plant. In this context, the seed encompasses all parts of the seed such as the seed coats, epidermal cells, seed cells, endosperm or embryonic tissue. The fine chemical produced in the process according to the invention may, however, also be isolated from the plant. In case of hentriacontane this can be in free form or bound to proteins. Fine chemical(s) produced by this process can be harvested by harvesting the non-human organisms either from the culture in which they grow or from the field. For example, this can be done via squeezing, grinding and/or extraction, salt precipitation and/or ion-exchange chromatography of the plant parts, preferably the plant seeds, plant fruits, plant tubers and the like.

[102949] [0437.1.7.20] to [0447.1.7.20] for the disclosure of these paragraphs see [0437.1.7.7] to [0447.1.7.7] above

[102950] The identity and purity of the isolated compound(s) can be determined by prior art techniques. These include high performance liquid chromatography (HPLC), spectroscopic methods, mass spectrometry (MS), staining methods, thin-layer chromatography, NIRS, enzyme assay or microbiological assays. These analytical methods are summarized for example in: Patek et al., Appl. Environ. Microbiol. 60, 133 (1994), Malakhova et al., Biotekhnologiya 11, 27 (1996); Schmidt et al., Bioprocess Engineer. 19, 67 (1998), Ullmann's Encyclopedia of Industrial Chemistry (1996) Vol. A27, VCH, Weinheim, pp. 89-90, pp. 521-540, pp. 540-547, pp. 559566, 575-581 and pp. 581-587, Michal G., “Biochemical Pathways: An Atlas of Biochemistry and Molecular Biology”, John Wiley and Sons (1999); Fallon A. et al. “Applications of HPLC in Biochemistry” in “Laboratory Techniques in Biochemistry and Molecular Biology”, Vol. 17 (1987).

[102951] The fine chemical obtained in the process are suitable as starting material for the synthesis of further products of value.

[102952] Accordingly, the present invention relates a method for the production of a pharmaceuticals, food stuff, animal feeds, nutrients or cosmetics comprising the steps of the process according to the invention, including the isolation of the fine chemical produced or a composition produced comprising the fine chemical, and, if desired, formulating the product with a pharmaceutical acceptable carrier or formulating the product in a form acceptable for an application in agriculture or formulating the product in a form acceptable for an application as food stuff, animal feed, nutrient or cosmetic. A further embodiment according to the invention is the use of the fine chemical produced in the process or of the transgenic non-human organisms in animal feeds, foodstuffs, medicines, food supplements, cosmetics or pharmaceuticals.

[102953] A further embodiment according to the invention is the use of the fine chemical long chain nalkanes (C20 to C40) or derivates like alcohols and acid of alkanes, optionally unsaturated as well as break down and/or cracking products of long chain alkanes, preferably long chain alkanes of C25 to C35, more preferably hentriacontane or waxes or components thereof produced in the process or of the transgenic non-human organisms as replacement for hydrocarbons from fossil sources.

[102954] [0450.1.7.20] to [0452.1.7.20] for the disclosure of these paragraphs see [0450.1.7.7] to [0452.1.7.7] above

[102955] Further in another embodiment, the present invention relates to the use of the nucleic acid molecule of the invention or used in the method of the invention alone or in combination with other genes of the respective fine chemical synthesis for example of the waxes and/or long chain alkanes biosynthesis, the polypeptide of the invention or used in the method of the invention, the nucleic acid construct of the invention, the expression cassette of the invention, the vector of the invention, the plant or plant tissue or the host cell of the invention, for the production of plant resistant to a herbicide inhibiting eventually the production of the fine chemical.

[102956] for the disclosure of this paragraph see [0454.1.7.7] above

[102957] In one embodiment, the present invention relates to a method for the identification of a gene product conferring the production of or an increase in the fine chemical production in a non-human cell, comprising the following steps:
[102958] (a) contacting, e.g. hybridizing, a, some or all nucleic acid molecules of a sample, e.g. cells, tissues, plants or microorganisms or a nucleic acid library, which can contain a candidate gene encoding a gene product conferring the production of or an increase in the fine chemical after expression, with the nucleic acid molecule of the present invention as shown in the respective line in column 5 or 8 of Table I A or B of application no. 20, preferably the coding region thereof, or a fragment or homolog thereof;
[102959] (b) identifying the nucleic acid molecules, which hybridize under relaxed stringent conditions with the nucleic acid molecule of the present invention in particular to the nucleic acid molecule sequence shown in the respective line in Table I, application no. 20, columns 5 or 8, preferably the coding region thereof, of a fragment or a homolog thereof, preferably in Table I B, application no. 20, columns 5 or 8, preferably the coding region thereof, of a fragment or a homolog thereof, and, optionally, isolating the full length cDNA clone or complete genomic clone;
[102960] (c) introducing the candidate nucleic acid molecules or a fragment thereof in host cells, preferably in a plant cell or a microorganism, appropriate for producing the fine chemical;
[102961] (d) expressing the identified nucleic acid molecules in the host cells for which the production of or the increased production of the fine chemical is desired;
[102962] (e) assaying the fine chemical level in the non-human host cells; and
[102963] (f) identifying the nucleic acid molecule and/or its gene product which expression confers the production of or an increase in the fine chemical level in the non-human host cell after expression compared to the wild type.

[102964] for the disclosure of this paragraph see [0456.1.7.7] above

[102965] In another embodiment, the present invention relates to a method for the identification of a gene product conferring the production of or an increase in the fine chemical production in a cell, comprising the following steps:
[102966] (a) identifying a nucleic acid molecule of a non-human organism, which is at least 20%, 25%, 30%, 35%, 40%, 50%, 60%, 70%, 80%, 90%, 95%, 96%, 97%, 98%, 99%, 99.5% or more homolog to the nucleic acid molecule encoding a polypeptide comprising the polypeptide molecule as shown in the respective line in column 5 or 8 of Table II, application no. 20, or comprising a consensus sequence or a polypeptide motif as shown in the respective line in column 8 of Table IV, application no. 20, or being encoded by a nucleic acid molecule comprising a polynucleotide as shown in the respective line in column 5 or 8 of Table I, application no. 20, preferably the coding region thereof, or a homologue thereof as described herein, for example via homology search in a data bank;
[102967] (b) introducing the candidate nucleic acid molecule in non-human host cells, preferably in a plant cell or a microorganism, appropriate for producing the fine chemical;
[102968] (c) expressing the identified nucleic acid molecule in the non-human host cells;
[102969] (d) assaying the fine chemical level in the non-human host cells; and
[102970] (e) identifying the nucleic acid molecule and its gene product which expression confers the production of or an increase in the fine chemical level in the non-human host cell after expression compared to the wild type.

[102971] for the disclosure of this paragraph see [0458.1.7.7] above

[102972] Further, the nucleic acid molecules disclosed herein, in particular the nucleic acid molecules shown in the respective line in column 5 or 8 of Table I A or B of application no. 20, may be sufficiently homologous to the sequences of related species such that these nucleic acid molecules may serve as markers for the construction of a genomic map in related organism or for association mapping. Furthermore natural variation in the genomic regions corresponding to nucleic acids disclosed herein, in particular the nucleic acid molecule shown in the respective line in column 5 or 8 of Table I A or B, of application no. 20 or homologous thereof, may lead to variation in the activity of the proteins disclosed herein, in particular the proteins comprising polypeptides as shown in the respective lines in column 5 or 8 of Table II A or B, of application no. 20 or their homologous, or comprising the consensus sequence or the polypeptide motif as shown in the respective line in column 8 of Table IV, and in consequence in a natural variation of an increased fine chemical production.

[102973] In consequence natural variation eventually also exists in form of more active allelic variants leading already to a relative increase in the fine chemical. Different variants of the nucleic acid molecules disclosed herein, in particular the nucleic acid molecules comprising the nucleic acid molecules as shown in the respective line in column 5 or 8 of Table I A or B of application no. 20, preferably the coding region thereof, which corresponds to different levels of increase in fine chemical, e.g. different levels of increase in fine chemical, can be indentified and used for marker assisted breeding for an enhanced production of the fine chemical.

[102974] Natural variation may also exist in the regulatory regions, e.g. the promoters of the natural genes comprising the nucleic acid molecules as shown in the respective line column 5 or 8 of Table I A or B of application no. 20, preferably the coding region thereof, which can similarly correspond to different levels of increase in the fine chemical, e.g. different levels of increase in the fine chemical can be identified and used for marker assisted breeding for an enhanced production of the fine chemical.

[102975] Accordingly, the present invention relates to a method for breeding plants for the production of the fine chemical, comprising
[102976] (a) selecting a first plant variety with an increased production of the fine chemical, e.g. based on an increased expression of a nucleic acid molecule of the invention as disclosed herein, in particular of a nucleic acid molecule comprising a nucleic acid molecule as shown in the respective line in column 5 or 8 of Table I A or B of application no. 20, preferably the coding region thereof, or fragments or homologs thereof, or a polypeptide comprising a polypeptide as shown in the respective line in column 5 or 8 of Table II A or B of application no. 20, or fragments or homologs thereof, or comprising a consensus sequence or a polypeptide motif as shown in the respective line in column 8 of Table IV, or a homolog thereof, as described herein;
[102977] (b) associating the level of increased production of the fine chemical, with the expression level or the genomic structure of a gene encoding said polypeptide or said nucleic acid molecule;
[102978] (c) crossing the first plant variety with a second plant variety, which significantly differs in its level of fine chemical; and
[102979] (d) identifying, which of the offspring varieties has got increased production of the fine chemical by the expression level of said polypeptide or nucleic acid molecule or the genomic structure of the gene(s) encoding said polypeptide or nucleic acid molecule of the invention.

[102980] [0462.1.7.20] to [0479.1.7.20] for the disclosure of these paragraphs see [0462.1.7.7] to [0479.1.7.7] above

[102981] In a further embodiment the nucleic acid molecule of the invention, the polypeptide of the invention, the nucleic acid construct of the invention, the expression cassette of the invention, the non-human organisms, the non-human host cell, the microorganisms, the plant, plant cell, or the part thereof, of the invention, the vector of the invention, the agonist identified with the method of the invention, the nucleic acid molecule identified with the method of the present invention, can be used for the production of the fine chemical or of the fine chemical and one or more other waxes and/or long chain alkanes.

[102982] Accordingly, the nucleic acid molecule of the invention, or the nucleic acid molecule identified with the method of the present invention or the complement sequences thereof, the polypeptide of the invention, the nucleic acid construct of the invention, the expression cassette of the invention, the non-human organism, the non-human host cell, the microorganism, the plant, plant cell, or the part thereof of the invention, the vector of the invention, the agonist identified with the method of the invention, the antibody of the present invention, can be used for the production of or the increase in the fine chemical in a non-human organism or part thereof, e.g. in a cell.

[102983] for the disclosure of this paragraph see [0481.1.7.7] above

[102984] for the disclosure of this paragraph see [0482.1.7.7] above

[102985] A special embodiment of this invention is related to genes which increase or generate the production of the fine chemical hentriacontane in plant cells, plants or part thereof. Phenotypes thereto are associated with yield of plants (=yield related phenotypes). In accordance with the invention, therefore, the respective genes identified in Table I, wherein in column 7 hentriacontane are mentioned, especially the coding region thereof, or homologs or fragments thereof,may be employed to enhance any yield-related phenotype. Increased yield may be determined in field trials of transgenic plants and suitable control plants. Alternatively, a transgene's ability to increase yield may be determined in a model plant. An increased yield phenotype may be determined in the field test or in a model plant by measuring any one or any combination of the following phenotypes, in comparison to a control plant: yield of dry harvestable parts of the plant, yield of dry aerial harvestable parts of the plant, yield of underground dry harvestable parts of the plant, yield of fresh weight harvestable parts of the plant, yield of aerial fresh weight harvestable parts of the plant yield of underground fresh weight harvestable parts of the plant, yield of the plant's fruit (both fresh and dried), grain dry weight, yield of seeds (both fresh and dry), and the like.

[102986] The most basic yield-related phenotype is increased yield associated with the presence of the gene or a homolog or a fragment thereof as a transgene in the plant, i.e., the intrinsic yield of the plant. Intrinsic yield capacity of a plant can be, for example, manifested in a field test or in a model system by demonstrating an improvement of seed yield (e.g. in terms of increased seed/grain size, increased ear number, increased seed number per ear, improvement of seed filling, improvement of seed composition, embryo and/or endosperm improvements, and the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under non-stressed conditions), improvement in plant architecture. Increased yield-related phenotypes may also be measured to determine tolerance to abiotic environmental stress. Abiotic stresses include drought, low temperature, salinity, osmotic stress, shade, high plant density, mechanical stresses, and oxidative stress, and yield-related phenotypes are encompassed by tolerance to such abiotic stresses. Additional phenotypes that can be monitored to determine enhanced tolerance to abiotic environmental stress include, without limitation, wilting; leaf browning; loss of turgor, which results in drooping of leaves or needles stems, and flowers; drooping and/or shedding of leaves or needles; the leaves are green but leaf angled slightly toward the ground compared with controls; leaf blades begun to fold (curl) inward; premature senescence of leaves or needles; loss of chlorophyll in leaves or needles and/or yellowing. Any of the yield-related phenotypes described above may be monitored in field tests or in model plants to demonstrate that a transgenic plant has increased tolerance to abiotic environmental stress. In accordance with the invention, the respective genes identified in Table 1, in case in column 7 hentriacontane is indicated, especially the coding region thereof, or homologs or fragments thereof, may be employed to enhance tolerance to abiotic environmental stress in a plant means that the plant, when confronted with abiotic environmental stress.

[102987] A polypeptide conferring an yield-increasing activity can be encoded by a respective nucleic acid sequence as shown in Table I, column 5 or 8, and/or comprises or consists of a respective polypeptide as depicted in Table II, column 5 and 8, and/or can be amplified with the respective primer set shown in Table III, column 8, in case in column 7 hentriacontane is indicated.

[102988] “Improved adaptation” to environmental stress like e.g. freezing and/or chilling temperatures refers to an improved plant performance under environmental stress conditions.

[102989] A modification, i.e. an increase, can be caused by endogenous or exogenous factors. For example, an increase in activity in an organism or a part thereof can be caused by adding a gene product or a precursor or an activator or an agonist to the media or nutrition or can be caused by introducing said subjects into an organism, transient or stable. Furthermore such an increase can be reached by the introduction of the respective inventive nucleic acid sequence or the encoded protein in the correct cell compartment for example into the nucleus or cytoplasmic respectively or into plastids either by transformation and/or targeting.

[102990] The term “yield” as used herein generally refers to a measurable produce from a plant, particularly a crop. Yield and yield increase (in comparison to a non-transformed starting or wild-type plant) can be measured in a number of ways, and it is understood that a skilled person will be able to apply the correct meaning in view of the particular embodiments, the particular crop concerned and the specific purpose or application concerned. The terms “improved yield” or “increased yield” can be used interchangeable.

[102991] As used herein, the term “improved yield” or the term “increased yield” means any improvement in the yield of any measured plant product, such as grain, fruit or fiber. In accordance with the invention, changes in different phenotypic traits may improve yield. For example, and without limitation, parameters such as floral organ development, root initiation, root biomass, seed number, seed weight, harvest index, tolerance to abiotic environmental stress, leaf formation, phototropism, apical dominance, and fruit development, are suitable measurements of improved yield. Any increase in yield is an improved yield in accordance with the invention. For example, the improvement in yield can comprise a 0.1%, 0.5%, 1%, 3%, 5%, 10%, 15%, 20%, 30%, 40%, 50%, 60%, 70%, 80%, 90% or greater increase in any measured parameter. For example, an increase in the bushel/acre yield of soybeans or corn derived from a crop comprising plants which are transgenic for the nucleotides and polypeptides of Table I, in case in column 7 hentriacontane is mentioned, as compared with the bushel/acre yield from untreated soybeans or corn cultivated under the same conditions, is an improved yield in accordance with the invention. The increased or improved yield can be achieved in the absence or presence of stress conditions.

[102992] For example, enhanced or increased “yield” refers to one or more yield parameters selected from the group consisting of biomass yield, dry biomass yield, aerial dry biomass yield, underground dry biomass yield, fresh-weight biomass yield, aerial fresh-weight biomass yield, underground fresh-weight biomass yield; enhanced yield of harvestable parts, either dry or fresh-weight or both, either aerial or underground or both; enhanced yield of crop fruit, either dry or fresh-weight or both, either aerial or underground or both; and preferably enhanced yield of seeds, either dry or fresh-weight or both, either aerial or underground or both.

[102993] Crop yield is defined herein as the number of bushels of relevant agricultural product (such as grain, forage, or seed) harvested per acre. Crop yield is impacted by abiotic stresses, such as drought, heat, salinity, and cold stress, and by the size (biomass) of the plant. Traditional plant breeding strategies are relatively slow and have in general not been successful in conferring increased tolerance to abiotic stresses. Grain yield improvements by conventional breeding have nearly reached a plateau in maize.

[102994] Accordingly, the yield of a plant can depend on the specific plant/crop of interest as well as its intended application (such as food production, feed production, processed food production, biofuel, biogas or alcohol production, or the like) of interest in each particular case. Thus, in one embodiment, yield is calculated as harvest index (expressed as a ratio of the weight of the respective harvestable parts divided by the total biomass), harvestable parts weight per area (acre, square meter, or the like); and the like. The harvest index, i.e., the ratio of yield biomass to the total cumulative biomass at harvest, in maize has remained essentially unchanged during selective breeding for grain yield over the last hundred years. Accordingly, recent yield improvements that have occurred in maize are the result of the increased total biomass production per unit land area. This increased total biomass has been achieved by increasing planting density, which has led to adaptive phenotypic alterations, such as a reduction in leaf angle, which may reduce shading of lower leaves, and tassel size, which may increase harvest index. Harvest index is relatively stable under many environmental conditions, and so a robust correlation between plant size and grain yield is possible. Plant size and grain yield are intrinsically linked, because the majority of grain biomass is dependent on current or stored photosynthetic productivity by the leaves and stem of the plant. As with abiotic stress tolerance, measurements of plant size in early development, under standardized conditions in a growth chamber or greenhouse, are standard practices to measure potential yield advantages conferred by the presence of a transgene.

[102995] Acccordingly, the yield of a plant can be increased by improving one or more of the yield-related phenotypes.

[102996] Such yield-related phenotypes or traits of a plant the improvement of which results in increased yield comprise, without limitation, the increase of the intrinsic yield capacity of a plant, improved nutrient use efficiency, and/or increased stress tolerance.

[102997] For example, yield refers to biomass yield, e.g. to dry weight biomass yield and/or fresh-weight biomass yield. Biomass yield refers to the aerial or underground parts of a plant, depending on the specific circumstances (test conditions, specific crop of interest, application of interest, and the like). In one embodiment, biomass yield refers to the aerial and underground parts. Biomass yield may be calculated as fresh-weight, dry weight or a moisture adjusted basis. Biomass yield may be calculated on a per plant basis or in relation to a specific area (e.g. biomass yield per acre/ square meter/ or the like).

[102998] In other embodiment, “yield” refers to seed yield which can be measured by one or more of the following parameters: number of seeds or number of filled seeds (per plant or per area (acre/square meter/ or the like)); seed filling rate (ratio between number of filled seeds and total number of seeds); number of flowers per plant; seed biomass or total seeds weight (per plant or per area (acre/square meter/ or the like); thousand kernel weight (TKW; extrapolated from the number of filled seeds counted and their total weight; an increase in TKW may be caused by an increased seed size, an increased seed weight, an increased embryo size, and/or an increased endosperm). Other parameters allowing to measure seed yield are also known in the art. Seed yield may be determined on a dry weight or on a fresh weight basis, or typically on a moisture adjusted basis, e.g. at 15.5 percent moisture.

[102999] For example, the term “increased yield” means that the plant, exhibits an increased growth rate, under conditions of abiotic environmental stress, compared to the corresponding wild-type plant.

[103000] An increased growth rate may be reflected inter alia by or confers an increased biomass production of the whole plant, or an increased biomass production of the aerial parts of a plant, or by an increased biomass production of the underground parts of a plant, or by an increased biomass production of parts of a plant, like stems, leaves, blossoms, fruits, and/or seeds.

[103001] Increased yield includes higher fruit yields, higher seed yields, higher fresh matter production, and/or higher dry matter production.

[103002] The term “increased yield” means that the plant, exhibits a prolonged growth under conditions of abiotic environmental stress, as compared to the corresponding, e.g. non-transformed, wild type organism. A prolonged growth comprises survival and/or continued growth of the plant, at the moment when the non-transformed wild type organism shows visual symptoms of deficiency and/or death.

[103003] When the plant of the invention is a corn plant increased yield for corn plants means, for example, increased seed yield, in particular for corn varieties used for feed or food. Increased seed yield of corn refers to an increased kernel size or weight, an increased kernel per ear, or increased ears per plant. Alternatively or in addition the cob yield may be increased, or the length or size of the cob is increased, or the kernel per cob ratio is improved.

[103004] When the plant of the invention is a soy plant, increased yield for soy plants means increased seed yield, in particular for soy varieties used for feed or food. Increased seed yield of soy refers for example to an increased kernel size or weight, an increased kernel per pod, or increased pods per plant.

[103005] When the plant of the invention is an oil seed rape (OSR) plant, increased yield for OSR plants means increased seed yield, in particular for OSR varieties used for feed or food. Increased seed yield of OSR refers to an increased seed size or weight, an increased seed number per silique, or increased siliques per plant.

[103006] When the plant of the invention is a cotton plant, increased yield for cotton plants means increased lint yield. Increased lint yield of cotton refers in one embodiment to an increased length of lint.

[103007] When the plant of the invention is a wheat plant increased yield for wheat plants means, for example, increased seed yield, in particular for wheat varieties used for feed or food. Increased seed yield of wheat refers to an increased kernel size or weight, an increased kernel per ear, or increased ears per plant. Increased seed yield of wheat refers also to an increased grain size or weight, an increased number of grains per spike, or increased number of spikes per plant. Alternatively or in addition increased seed yield of wheat refers also to an increased grain yield, increased length of spikes, increased number of spikelets per spike , increased 1000 grain weight or increased flag leaf area (flag leaf is the leaf directely below the spike).

[103008] Said increased yield can typically be achieved by enhancing or improving, one or more yield-related traits of the plant. Such yield-related traits of a plant comprise, without limitation, the increase of the intrinsic yield capacity of a plant, improved nutrient use efficiency, and/or increased stress tolerance, in particular increased abiotic stress tolerance.

[103009] Intrinsic yield capacity of a plant can be, for example, manifested by improving the specific (intrinsic) seed yield (e.g. in terms of increased seed/ grain size, increased ear number, increased seed number per ear, improvement of seed filling, improvement of seed composition, embryo and/or endosperm improvements, or the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under stressed or non-stressed conditions), improvement in plant architecture, cell cycle modifications, photosynthesis modifications, various signaling pathway modifications, modification of transcriptional regulation, modification of translational regulation, modification of enzyme activities, and the like); and/or the like.

[103010] The improvement or increase of stress tolerance of a plant can for example be manifested by improving or increasing a plant's tolerance against stress, particularly abiotic stress. In the present application, abiotic stress refers generally to abiotic environmental conditions a plant is typically confronted with, including, but not limited to, drought (tolerance to drought may be achieved as a result of improved water use efficiency), heat, low temperatures and cold conditions (such as freezing and chilling conditions), salinity, osmotic stress , shade, high plant density, mechanical stress, oxidative stress, and the like.

[103011] The increased plant yield can also be mediated by increasing the “nutrient use efficiency of a plant”, e.g. by improving the use efficiency of nutrients including, but not limited to, phosphorus, potassium, and nitrogen. Further, higher yields may be obtained with current or standard levels of nitrogen use.

[103012] Generally, the term “increased tolerance to stress” can be defined as survival of plants, and/or higher yield production, under stress conditions as compared to a non-transformed wild type or starting plant: For example, the plant of the invention or produced according to the method of the invention is better adapted to the stress conditions. “Improved adaptation” to environmental stress like e.g. drought, heat, nutrient depletion, freezing and/or chilling temperatures refers herein to an improved plant performance resulting in an increased yield, particularly with regard to one or more of the yield related traits as defined in more detail above.

[103013] During its life-cycle, a plant is generally confronted with a diversity of environmental conditions. Any such conditions, which may, under certain circumstances, have an impact on plant yield, are herein referred to as “stress” condition. Environmental stresses may generally be divided into biotic and abiotic (environmental) stresses. Unfavorable nutrient conditions are sometimes also referred to as “environmental stress”. The present invention does also contemplate solutions for this kind of environmental stress, e.g. referring to increased nutrient use efficiency.

[103014] For the purposes of the description of the present invention, the terms “enhanced tolerance to abiotic stress”, “enhanced resistance to abiotic environmental stress”, “enhanced tolerance to environmental stress”, “improved adaptation to environmental stress” and other variations and expressions similar in its meaning are used interchangeably and refer, without limitation, to an improvement in tolerance to one or more abiotic environmental stress(es) as described herein and as compared to a corresponding origin or wild type plant or a part thereof.

[103015] The term abiotic stress tolerance(s) refers for example low temperature tolerance, drought tolerance or improved water use efficiency (WUE), heat tolerance, salt stress tolerance and others. Studies of a plant's response to desiccation, osmotic shock, and temperature extremes are also employed to determine the plant's tolerance or resistance to abiotic stresses. Water use efficiency (WUE) is a parameter often correlated with drought tolerance. In selecting traits for improving crops, a decrease in water use, without a change in growth would have particular merit in an irrigated agricultural system where the water input costs were high. An increase in growth without a corresponding jump in water use would have applicability to all agricultural systems. In many agricultural systems where water supply is not limiting, an increase in growth, even if it came at the expense of an increase in water use also increases yield.

[103016] Drought stress means any environmental stress which leads to a lack of water in plants or reduction of water supply to plants, including a secondary stress by low temperature and/or salt, and/or a primary stress during drought or heat, e.g. desiccation etc.

### Specific Embodiments

[103017] Accordingly, this invention provides respective measures and methods to produce plants with increased yield, e.g. genes conferring an increased yield-related trait, for example enhanced tolerance to abiotic environmental stress, for example an increased drought tolerance and/or low temperature tolerance and/or an increased nutrient use efficiency, intrinsic yield and/or another increased yield-related trait, upon expression or over-expression. Accordingly, the present invention provides such genes in case in column 7 of the respective Table or hentriacontane is indicated. In particular, such genes are described in column 5 as well as in column 8 of Tables I, especially the coding region thereof, or homologs or fragments thereof, in case hentriacontane are indicated in column 7 or the respective polypeptides are described in column 5 as well as in column 8 of Table II, or homologs or fragments thereof, in case hentriacontane are indicated in column 7.

[103018] Accordingly, the present invention provides respective transgenic plants showing one or more improved yield-related traits as compared to the corresponding origin or the wild type plant and methods for producing such transgenic plants with increased yield in case in column 7 of the respective Table “hentriacontane” is indicated.

[103019] In one embodiment, one or more of said yield-increasing activities are increased by increasing the amount and/or the specific activity of one or more proteins listed in Table II, column 5 or 8 in a compartment of a cell indicated in Table II, column 6, in case in column 7 of the respective Table “hentriacontane” is indicated.

[103020] Accordingly to present invention, the yield of the plant of the invention is increased by improving one or more of the yield-related traits as defined herein. Said increased yield in accordance with the present invention can typically be achieved by enhancing or improving, in comparison to an origin or wild-type plant, one or more yield-related traits of said plant. Such yield-related traits of a plant the improvement of which results in increased yield comprise, without limitation, the increase of the intrinsic yield capacity of a plant, improved nutrient use efficiency, like nitrogen use efficiency, and/or increased stress tolerance.

[103021] In one embodiment, the present invention provides respective transgenic plants showing one or more improved yield-related traits, preferably increased drought tolerance as compared to the corresponding origin or the wild type plant and methods for producing such transgenic plants with increased yield in case in column 7 of the respective Table “hentriacontane” is indicated.

[103022] In a further embodiment the present invention is characterized by the features as disclosed in the following items:

[103023] Item 1. A process for the production of a fine chemical selected from the group consisting of:

[103024] hentriacontane
[103025] (a) increasing or generating one or more activities selected from the group consisting of 3-deoxy-7-phosphoheptulonate synthase, acid shock protein, amino-acid acetyltransferase, assembly protein for periplasmic nitrate reductase, At2g45420-protein, At5g43630-protein, ATP-dependent RNA helicase, b0833-protein, b1259-protein, b1445-protein, b1670-protein, b1930-protein, b2399-protein, b2653-protein, b3644-protein, cell division protein, cyclin D, cysteine synthase A, dihydroxyacetone kinase, electron transport complex protein, ethanolamine utilization protein, exonuclease subunit, fumarylacetoacetate hydrolase, GDP-mannose 4,6-dehydratase, inner membrane protein, ion-transport protein, L-asparaginase, MADS box protein transcription factor, mechanosensitive ion channel, metal-dependent hydrolase, methionine permease, O-antigen chain length determinant, ornithine decarboxylase, orotidine-5′-phosphate decarboxylase, peptide chain release factor, phospho-2-dehydro-3-deoxyheptonate aldolase, phosphoadenosine phosphosulfate reductase, phosphoglycolate phosphatase, pyrimidine-specific ribonucleoside hydrolase, pyruvate kinase, RNA-binding protein, S-adenosyl-L-methionine-dependent methyltransferase, serine protease, short-chain alcohol dehydrogenase family, sigma-54 dependent response regulator, spermidine synthase, sugar transport system permease protein, transcription factor, transcriptional regulator, YFL019C-protein, YHL039W-protein, YHR022C-A-protein, YJL127W-A-protein, ylr157c-protein, ynl321w-protein, ypr170c-protein, and YPR172W-protein, in a non-human organism or a part thereof, preferably a microorganism, a plant cell, a plant or a part thereof, as compared to a corresponding non-transformed wild type non-human organism or a part thereof; and
[103026] (b) growing the non-human organism or a part thereof under conditions which permit the production of hentriacontane or a composition comprising hentriacontane in said non-human organism or in the culture medium surrounding said non-human organism.

[103027] Item 2. A process for the production of a fine chemical selected from the group consisting of: hentriacontane, which comprises
[103028] (A)
   [103029] (i) increasing or generating of the expression of; and/or
   [103030] (ii) increasing or generating the expression of an expression product of; and/or
   [103031] (iii) increasing or generating one or more activities of an expression product encoded by;
      at least one respective nucleic acid molecule comprising a nucleic acid molecule selected from the group consisting of:
   [103032] (a) a nucleic acid molecule encoding the polypeptide shown in column 5 or 8 of Table II, preferably Table II B, application no.20, or a homolog or a fragment thereof;
   [103033] (b) a nucleic acid molecule shown in column 5 or 8 of Table I, preferably Table I B, application no.20, or a homolog or a fragment thereof (preferably the coding region thereof);
   [103034] (c) a nucleic acid molecule, which, as a result of the degeneracy of the genetic code, can be derived from a polypeptide sequence depicted in column 5 or 8 of Table II, preferably Table II B, application no.20;
   [103035] (d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 20, or the coding region thereof;
   [103036] (e) a nucleic acid molecule encoding a polypeptide having at least 30%, in particular 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
   [103037] (f) a nucleic acid molecule which hybridizes with a nucleic acid molecule of (a), (b), (c), (d) or (e) under stringent hybridization conditions;
   [103038] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
   [103039] (h) a nucleic acid molecule encoding a polypeptide comprising the consensus sequence or one or more polypeptide motifs as shown in column 8 of Table IV, application no.20;
   [103040] (i) a nucleic acid molecule encoding a polypeptide having the activity represented by a respective protein as depicted in column 5 of Table II, application no. 20;
   [103041] (j) a nucleic acid molecule which comprises a polynucleotide, which is obtained by amplifying a cDNA library or a genomic library using the primers as depicted in column 8 of Table III, application no. 20; and
   [103042] (k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
   [103043] or a nucleic acid molecule comprising a sequence which is complementary thereto;
      in a non-human organism or a part thereof, preferably a microorganism, a plant cell, a plant or a part thereof, as compared to a corresponding non-transformed wild type non-human organism or a part thereof; and
[103044] (B) growing the non-human organism or a part thereof under conditions which permit the production of hentriacontane or a composition comprising hentriacontane in said non-human organism or in the culture medium surrounding said non-human organism.

[103045] Item 3. A process of items 1 or 2, comprising of recovering hentriacontane in its free or bound form.

[103046] Item 4. The process of any one of items 1 to 3, comprising the following steps:
[103047] (a) selecting a non-human organism or a part thereof expressing a polypeptide encoded by the nucleic acid molecule characterized in item 2;
[103048] (b) mutagenizing the selected non-human organism or the part thereof;
[103049] (c) comparing the activity or the expression level of said polypeptide in the mutagenized non-human organism or the part thereof with the activity or the expression of said polypeptide of the selected non-human organisms or the part thereof;
[103050] (d) selecting the mutated non-human organisms or parts thereof, which comprise an increased activity or expression level of said polypeptide compared to the selected non-human organism or the part thereof;
[103051] (e) optionally, growing and cultivating the non-human organisms or the parts thereof; and
[103052] (f) recovering, and optionally isolating, the free or bound hentriacontane produced by the selected mutated non-human organisms or parts thereof.

[103053] Item 5. The process of any one of items 1 to 4 wherein the activity of said protein or the expression of said nucleic acid molecule is increased or generated transiently or stably.

[103054] Item 6. An isolated nucleic acid molecule comprising a nucleic acid molecule selected from the group consisting of:
[103055] (a) a nucleic acid molecule encoding a polypeptide shown in Table II, application no. 20, column 5 or 8, preferably shown in Table II B, application no. 20, column 8, or a homolog or a fragment thereof;
[103056] (b) a nucleic acid molecule shown in Table I, application no. 20, column 5 or 8, preferably shown in Table I B, application no. 20, column 8, preferably the coding region thereof, or a homolog or a fragment thereof;
[103057] (c) a nucleic acid molecule, which, as a result of the degeneracy of the genetic code, can be derived from a polypeptide sequence depicted in column 5 or 8 of Table II, application no. 20, preferably in column 8 of Table II B, application no. application no.;
[103058] (d) a nucleic acid molecule having at least 30% identity, preferably at least 40%, 50%, 60%, 70%, 75%, 80%, 85%, 90%, 95%, 96%, 97%, 98%, 99%, 99.5%, with the nucleic acid molecule sequence of a polynucleotide comprising the nucleic acid molecule shown in column 5 or 8 of Table I, application no. 3, preferably shown in column 8 of Table I B, application no. 20, preferably the coding region thereof;
[103059] (e) a nucleic acid molecule encoding a polypeptide having at least 30% identity, preferably at least 40%, 50% 60%, 70%, 75%, 80%, 85%, 90%, 95%, 96%, 97%, 98%, 99%, 99.5%, identity with the amino acid sequence of the polypeptide encoded by the nucleic acid molecule of (a), (b), (c) or (d);
[103060] (f) a nucleic acid molecule which hybridizes with a nucleic acid molecule of (a), (b), (c), (d) or (e) under stringent hybridisation conditions;
[103061] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
[103062] (h) a nucleic acid molecule encoding a polypeptide comprising the consensus sequence or one or more polypeptide motifs as shown in column 8 of Table IV, application no. 20;
[103063] (i) a nucleic acid molecule encoding a polypeptide having the activity represented by a respective protein as depicted in column 5 of Table II, application no. 20;
[103064] (j) a nucleic acid molecule comprising a polynucleotide which is obtained by amplifying a cDNA library or a genomic library using the primers in Table III, application no. 20, column 8;
[103065] and
[103066] (k) nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, preferably 20 nt, 30 nt, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
[103067] or a nucleic acid molecule comprising a sequence which is complementary thereto.

[103068] Item 7. A nucleic acid construct which confers the expression of the nucleic acid molecule as defined in item 6, comprising one or more regulatory elements.

[103069] Item 8. An expression cassette comprising
[103070] (a) a promoter, preferably selected from the group consisting of Big35S, PCUbi, Super and USP;
[103071] (b) a nucleic acid molecule as defined in item 6.

[103072] Item 9. A vector comprising the nucleic acid molecule as defined in item 6 or the nucleic acid construct as claimed in item 7 or the expression cassette as claimed in item 8.

[103073] Item 10. A host cell or a plant organelle, which has been transformed with the vector as claimed in item 9 or the nucleic acid molecule as defined in item 6 or the nucleic acid construct as claimed in item 7 or the expression cassette as claimed in item 8.

[103074] Item 11. The host cell of item 10, which is a plant cell or a microorganism.

[103075] Item 12. A process for producing a polypeptide, wherein the polypeptide is expressed in a host cell as claimed in item 10 or 11.

[103076] Item 13. A polypeptide produced by the process as claimed in item 12 or encoded by the nucleic acid molecule as defined in item 6.

[103077] Item 14. An antibody, which binds specifically to the polypeptide as claimed in item 13.

[103078] Item 15. A plant tissue, propagation material, harvested material or a plant comprising the host cell as claimed in item 10 or 11.

[103079] Item 16. A process for the identification of a compound conferring an increase in hentriacontane production in a non-human organism, comprising the steps:
[103080] (a) culturing a plant cell or tissue or microorganism or maintaining a plant expressing the polypeptide encoded by the nucleic acid molecule as defined in item 6 conferring an increase in the amount of hentriacontane in a non-human organism or a part thereof and a readout system capable of interacting with the polypeptide under suitable conditions which permit the interaction of the polypeptide with said readout system in the presence of a compound or a sample comprising a plurality of compounds and capable of providing a detectable signal in response to the binding of a compound to said polypeptide under conditions which permit the expression of said readout system and of the polypeptide encoded by the nucleic acid molecule of item 6 conferring an increase in the amount of hentriacontane in a non-human organism or a part thereof;
[103081] (b) identifying if the compound is an effective agonist by detecting the presence or absence or increase of a signal produced by said readout system.

[103082] Item 17. A method for the production of an agricultural composition comprising the steps of the method of item 16 and formulating the compound identified in item 16 in a form acceptable for an application in agriculture.

[103083] Item 18. A composition comprising the nucleic acid molecule as defined in item 6, the polypeptide as claimed in item 13, the nucleic acid construct as claimed in item 7, the expression cassette as claimed in item 8, the vector as claimed in item 9, the compound as claimed in item 16, the antibody as claimed in item 14, and optionally an agricultural acceptable carrier.

[103084] Item 19. Use of the nucleic acid molecule as defined in item 6 for the identification of a nucleic acid molecule conferring an increase in hentriacontane after expression.

[103085] Item 20. Cosmetic, pharmaceutical, food or feed composition comprising the nucleic acid moleculeas defined in item 6, the polypeptide as claimed in item 13, the nucleic acid construct as claimed in item 7, the expression cassette as claimed in item 8, the vector as claimed in item 9, the antibody as claimed in item 14 , the plant or plant tissue as claimed in item 15, the harvested material as claimed in item 15 or the host cell of item as claimed in items 10 or 11.

[103086] Item 21. Use of the nuclei acid molecule as defined in item 6, the polypeptide as claimed in item 13, the nucleic acid construct as claimed in item 7, the expression cassette as claimed in item 8, the vector as claimed in item 9, the plant or plant tissue as claimed in item 15, or the host cell of item 10 to 11 for the production of plant resistant to a herbicide inhibiting the production of hentriacontane.

[103087] Item 22. A method for producing a plant with increased yield as compared to a corresponding non-transformed wild type plant, whereby the method comprises
[103088] (i) increasing or generating of the expression of; and/or
[103089] (ii) increasing or generating the expression of an expression product of; and/or
[103090] (iii) increasing or generating one or more activities of an expression product encoded by;
   at least one respective nucleic acid molecule comprising a nucleic acid molecule selected from the group consisting of:
[103091] (a) a nucleic acid molecule encoding the polypeptide shown in column 5 or 8 of Table II, preferably Table II B, application no. 20, or a homolog or a fragment thereof, in case in column 7 hentriacontane is indicated;
[103092] (b) a nucleic acid molecule shown in column 5 or 8 of Table I, preferably Table I B, application no. 20, or a homolog or a fragment thereof (preferably the coding region thereof) , in case in column 7 hentriacontane is indicated;
[103093] (c) a nucleic acid molecule, which, as a result of the degeneracy of the genetic code, can be derived from a polypeptide sequence depicted in column 5 or 8 of Table II, preferably Table II B, application no. 20, in case in column 7 hentriacontane is indicated;
[103094] (d) a nucleic acid molecule having at least 30%, in particular at least 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, application no. 20, or the coding region thereof, in case in column 7 hentriacontane is indicated;
[103095] (e) a nucleic acid molecule encoding a polypeptide having at least 30%, in particular 50% , 60%, 70%, 80%, 85%, 90%, 95%, 97%, 98%, 99% or 99.5% identity with the amino acid sequence of the polypeptide encoded by the nucleic acid molecule of (a), (b), (c) or (d);
[103096] (f) a nucleic acid molecule which hybridizes with a nucleic acid molecule of (a), (b), (c), (d) or (e) under stringent hybridization conditions;
[103097] (g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
[103098] (h) a nucleic acid molecule encoding a polypeptide comprising the consensus sequence or one or more polypeptide motifs as shown in column 8 of Table IV, application no. 20, in case in column 7 hentriacontane is indicated;
[103099] (i) a nucleic acid molecule encoding a polypeptide having the activity represented by a respective protein as depicted in column 5 of Table II, application no. 20, in case in column 7 hentriacontane is indicated;
[103100] (j) a nucleic acid molecule which comprises a polynucleotide, which is obtained by amplifying a cDNA library or a genomic library using the primers as depicted in column 8 of Table III, application no. 20, in case in column 7 hentriacontane is indicated; and
[103101] (k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, preferably 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
[103102] or a nucleic acid molecule comprising a sequence which is complementary thereto;
   in a plant cell, plant or a part thereof.

[103103] Item 23. A method for producing a plant with increased yield according to item 22, which comprises in addition the regeneration of a plant from such transformed plant cell, plant cell compartment, plant cell nucleus, plant cell or plant tissue with increased yield.

[103104] [0483.1.7.20] to [0492.1.7.20] for the disclosure of these paragraphs see [0483.1.7.7] to [0492.1.7.7] above

### Example 10

### Purification of a Fine Chemical e.g. Waxes and/or Long Chain Alkanes, e.g. Hentriacontane

[103105] The material, such as microorganism s or plants, can be disrupted by sonication, grinding in a glass mill, liquid nitrogen and grinding, cooking, or via other applicable methods. After disruption centrifugation may follow. The waxes and/or long chain alkanes are extracted with petroleum ether and the solvent is evaporated lateron.

[103106] Identity and purity of the waxes and/or long chain alkanes isolated can be determined by standard techniques of the art. They encompass high-performance liquid chromatography (HPLC), spectroscopic methods, mass spectrometry (MS), staining methods, thin-layer chromatography, NIRS, enzyme assay or microbiological assays. These analytical methods are compiled in: Patek et al. (1994) Appl. Environ. Microbiol. 60: 133-140; Malakhova et al. (1996) Biotekhnologiya 11: 27-32; and Schmidt et al. (1998) Bioprocess Engineer. 19: 67-70. Ulmann's Encyclopedia of Industrial Chemistry (1996) Vol. A27, VCH: Weinheim, pp. 89-90, pp. 521-540, pp. 540547, pp. 559-566, 575-581 and pp. 581-587; Michal, G (1999) Biochemical Pathways: An Atlas of Biochemistry and Molecular Biology, John Wiley and Sons; Fallon, A. et al. (1987) Applications of HPLC in Biochemistry in: Laboratory Techniques in Biochemistry and Molecular Biology, Vol. 17.

[103107] for the disclosure of this paragraph see [0494.1.7.7] above

### Example 11a

### PCR Amplification of the Sequences

[103108] Unless otherwise specified, standard methods as described in Sambrook et al., Molecular Cloning: A laboratory manual, Cold Spring Harbor 1989, Cold Spring Harbor Laboratory Press are used.

[103109] The inventive sequences as shown in the respective line in Table I, column 5, preferably the coding region thereof, were amplified by PCR as described in the protocol of the Pfu Ultra, Pfu Turbo or Herculase DNA polymerase (Stratagene). The composition for the protocol of the Pfu Ultra, Pfu Turbo or Herculase DNA polymerase was as follows: 1×PCR buffer (Stratagene), 0.2 mM of each dNTP, 100 ng genomic DNA of Saccharomyces cerevisiae (strain S288C; Research Genetics, Inc., now Invitrogen), Escherichia coli (strain MG1655; E. coli Genetic Stock Center), Synechocystis sp. (strain PCC6803), Azotobacter vinelandii (strain N.R. Smith,16), Thermus thermophilus (HB8) or 50 ng cDNA from various tissues and development stages of Arabidopsis thaliana (ecotype Columbia), Physcomitrella patens, Glycine max (variety Resnick), Brassica napus, Oryza sativa or Zea mays (variety B73, Mo17, A188), 50 pmol forward primer, 50 pmol reverse primer, with or without 1 M Betaine, 2.5 u Pfu Ultra, Pfu Turbo or Herculase DNA polymerase.

[103110] The amplification cycles were as follows:

[103111] 1 cycle of 2-3 minutes at 94-95° C., then 25-36 cycles with 30-60 seconds at 94-95° C., 30-45 seconds at 50-60° C. and 210-480 seconds at 72° C., followed by 1 cycle of 5-10 minutes at 72° C., then 4-16° C.—preferably for Saccharomyces cerevisiae, Escherichia coli, Synechocystis sp., Azotobacter vinelandii, Thermus thermophilus.

[103112] In case of Arabidopsis thaliana, Brassica napus, Glycine max, Oryza sativa, Physcomitrella patens, Zea mays the amplification cycles were as follows: 1 cycle with 30 seconds at 94° C., 30 seconds at 61° C., 15 minutes at 72° C., then 2 cycles with 30 seconds at 94° C., 30 seconds at 60° C., 15 minutes at 72° C., then 3 cycles with 30 seconds at 94° C., 30 seconds at 59° C., 15 minutes at 72° C., then 4 cycles with 30 seconds at 94° C., 30 seconds at 58° C., 15 minutes at 72° C., then 25 cycles with 30 seconds at 94° C., 30 seconds at 57° C., 15 minutes at 72° C., then 1 cycle with 10 minutes at 72° C., then finally 4-16° C.

[103113] RNAs were generated with the RNeasy Plant Kit according to the standard protocol (Qiagen) and Superscript II Reverse Transkriptase was used to produce double stranded cDNA according to the standard protocol (Invitrogen).

[103114] ORF specific primer pairs for the genes to be expressed are shown in the respective line in Table III, column 8. The following adapter sequences were added to Saccharomyces cerevisiae ORF specific primers (see Table III) for cloning purposes:

SEQ ID NO: 20

| | | |
|---|---|---|
| i) | foward primer: | 5′-GGAATTCCAGCTGACCACC-3′ |

SEQ ID NO: 21

| | | |
|---|---|---|
| ii) | reverse primer: | 5′-GATCCCCGGGAATTGCCATG-3′ |

[103115] These adaptor sequences allow cloning of the ORF into the various vectors containing the Resgen adaptors, see table column E of Table c.

[103116] The following adapter sequences were added to Saccharomyces cerevisiae, Escherichia coli, Synechocystis sp., Azotobacter vinelandii, Thermus thermophilus, Arabidopsis thaliana, Brassica napus, Glycine max, Oryza sativa , Physcomitrella patens, or Zea mays ORF specific primers for cloning purposes:

| SEQ ID NO: 22 | | |
|---|---|---|
| iii) | forward primer: | 5′-TTGCTCTTCC-3′ |
| SEQ ID NO: 23 | | |
| iiii) | reverse primer: | 5′-TTGCTCTTCG-3′ |

[103117] The adaptor sequences allow cloning of the ORF into the various vectors containing the Colic adaptors, see table column E of Table c.

[103118] Therefore for amplification and cloning of Saccharomyces cerevisiae SEQ ID NO: 64144, a primer consisting of the adaptor sequence i) and the ORF specific sequence SEQ ID NO: 64146 and a second primer consisting of the adaptor sequence ii) and the ORF specific sequence SEQ ID NO: 64147 were used.

[103119] For amplification and cloning of Escherichia coli SEQ ID NO: 7081, a primer consisting of the adaptor sequence iii) and the ORF specific sequence SEQ ID NO: 7261 and a second primer consisting of the adaptor sequence iiii) and the ORF specific sequence SEQ ID NO: 7262 were used.

[103120] For amplification and cloning of Synechocystis sp. SEQ ID NO: 59370, a primer consisting of the adaptor sequence iii) and the ORF specific sequence SEQ ID NO: 59846 and a second primer consisting of the adaptor sequence iiii) and the ORF specific sequence SEQ ID NO: 59847 were used.

[103121] For amplification and cloning of Arabidopsis thaliana SEQ ID NO: 1623, a primer consisting of the adaptor sequence iii) and the ORF specific sequence SEQ ID NO: 1691 and a second primer consisting of the adaptor sequence iiii) and the ORF specific sequence SEQ ID NO: 1692 were used.

[103122] For amplification and cloning of Azotobacter vinelandii SEQ ID NO: 29286, a primer consisting of the adaptor sequence iii) and the ORF specific sequence SEQ ID NO: 29394 and a second primer consisting of the adaptor sequence iiii) and the ORF specific sequence SEQ ID NO: 29395 were used.

[103123] For amplification and cloning of Glycine max SEQ ID NO: 46850, a primer consisting of the adaptor sequence iii) and the ORF specific sequence SEQ ID NO: 47020 and a second primer consisting of the adaptor sequence iiii) and the ORF specific sequence SEQ ID NO: 47021 were used.

[103124] For amplification and cloning of Thermus thermophilus SEQ ID NO: 60301, a primer consisting of the adaptor sequence iii) and the ORF specific sequence SEQ ID NO: 60849 and a second primer consisting of the adaptor sequence iiii) and the ORF specific sequence SEQ ID NO: 60850 were used.

[103125] Following these examples every sequence disclosed in Table I, preferably column 5, especially the coding region thereof can be cloned by fusing the adaptor sequences to the respective specific primers sequences as disclosed in Table III, column 8 using the respective vectors shown in Table c.

[103126] [0496.1.7.20] to [0499.1.7.20] for the disclosure of these paragraphs see [0496.1.7.7] to [0499.1.7.7] above

### Example 11e

### Cloning of Inventive Sequences as Shown in Table I, Column 5 in the Different Expression Vectors

[103127] For cloning of for example SEQ ID NO: 7081 from Escherichia coli or any other ORF from Escherichia coli, Synechocystis sp., Azotobacter vinelandii, Thermus thermophilus, Arabidopsis thaliana, or Glycine max the vector DNA was treated with the restriction enzymes Pacl and Ncol following the standard protocol (MBI Fermentas).

[103128] For cloning for example the ORFs of SEQ ID NO: 64144 from Saccharomyces cerevisiae or any other ORF from Saccharomyces cerevisiae into vectors containing the Resgen adaptor sequence the respective vector DNA was treated with the restriction enzyme Ncol.

[103129] In all cases the reaction was stopped by inactivation at 70° C. for 20 minutes and purified over QlAquick or NucleoSpin Extract II columns following the standard protocol (Qiagen or Macherey-Nagel).

[103130] Then the PCR-product representing the amplified ORF with the respective adapter sequences and the vector DNA were treated with T4 DNA polymerase according to the standard protocol (MBI Fermentas) to produce single stranded overhangs with the parameters 1 unit T4 DNA polymerase at 37° C. for 2-10 minutes for the vector and 1-2 u T4 DNA polymerase at 15-17° C. for 10-60 minutes for the PCR product representing SEQ ID NO: 7081.

[103131] The reaction was stopped by addition of high-salt buffer and purified over QlAquick or NucleoSpin Extract II columns following the standard protocol (Qiagen or Macherey-Nagel).

[103132] According to this example the skilled person is able to clone all sequences disclosed in Table I, preferably column 5 or column 8, especially the coding region thereof.

[103133] Approximately 30-60 ng of prepared vector and a defined amount of prepared amplificate were mixed and hybridized at 65° C. for 15 minutes followed by 37° C. 0.1° C/1 seconds, followed by 37° C. 10 minutes, followed by 0.1° C/1 seconds, then 4-10 ° C.

[103134] The ligated constructs were transformed in the same reaction vessel by addition of competent E. coli cells (strain DH5alpha) and incubation for 20 minutes at 1° C. followed by a heat shock for 90 seconds at 42° C. and cooling to 1-4° C. Then, complete medium (SOC) was added and the mixture was incubated for 45 minutes at 37° C. The entire mixture was subsequently plated onto an agar plate with 0.05 mg/ml kanamycin and incubated overnight at 37° C.

[103135] The outcome of the cloning step was verified by amplification with the aid of primers which bind upstream and downstream of the integration site, thus allowing the amplification of the insertion.

[103136] The amplifications were carried out as described in the protocol of Taq DNA polymerase (Gibco-BRL).

[103137] The amplification cycles were as follows:

[103138] 1 cycle of 1-5 minutes at 94° C., followed by 35 cycles of in each case 15-60 seconds at 94° C., 15-60 seconds at 50-66° C. and 5-15 minutes at 72° C., followed by 1 cycle of 10 minutes at 72° C., then 4-16° C.

[103139] Several colonies were checked, but only one colony for which a PCR product of the expected size was detected was used in the following steps.

[103140] A portion of this positive colony was transferred into a reaction vessel filled with complete medium (LB) supplemented with kanamycin and incubated overnight at 37° C.

[103141] The plasmid preparation was carried out as specified in the Qiaprep or NucleoSpin Multi-96 Plus standard protocol (Qiagen or Macherey-Nagel).

[103142] [0501.1.7.20] to [0503.1.7.20] for the disclosure of these paragraphs see [0501.1.7.7] to [0503.1.7.7] above

[103143] Table d showing results of plant analyses

TABLE d

| SeqID | Target | Locus | Metabolite | Source | Promotor | Method | Min | Max |
|---|---|---|---|---|---|---|---|---|
| 113714 | plastidic | At2g36580 | hentriacontane | ARA_LEAF | p-PcUBI | GC | 28 | 48 |
| 108727 | non-targ | At2g42830 | hentriacontane | ARA_LEAF | p-PcUBI | GC | 49 | 90 |
| 1623 | non-targ | At2g45420 | hentriacontane | ARA_LEAF | p-PcUBI | GC | 34 | 100 |
| 109717 | non-targ | At4g09960 | hentriacontane | ARA_LEAF | p-PcUBI | GC | 27 | 231 |
| 142929 | non-targ | At5g43630 | hentriacontane | ARA_LEAF | p-PcUBI | GC | 26 | 37 |
| 70006 | non-targ | At5g60110 | hentriacontane | ARA_LEAF | p-PcUBI | GC | 28 | 97 |
| 29286 | non-targ | Avin- | hentriacontane | ARA_LEAF | p-PcUBI | GC | 55 | 116 |
| | | DRAFT_3209 | | | | | | |
| 32308 | non-targ | Avin- | hentriacontane | ARA_LEAF | p-PcUBI | GC | 26 | 53 |
| | | DRAFT_5292 | | | | | | |
| 154509 | plastidic | B0121 | hentriacontane | ARA_LEAF | p-Super | GC | 25 | 50 |
| 7081 | non-targ | B0161 | hentriacontane | ARA_LEAF | p-Super | GC | 26 | 69 |
| 155230 | non-targ | B0236 | hentriacontane | ARA_LEAF | p-Super | GC | 28 | 59 |
| 155370 | non-targ | B0577 | hentriacontane | ARA_LEAF | p-Super | GC | 26 | 65 |
| 143954 | non-targ | B0651 | hentriacontane | ARA_LEAF | p-Super | GC | 26 | 49 |
| 155613 | non-targ | B0787 | hentriacontane | ARA_LEAF | p-Super | GC | 24 | 107 |
| 155657 | non-targ | B0833 | hentriacontane | ARA_LEAF | p-Super | GC | 24 | 48 |
| 155665 | plastidic | B0921 | hentriacontane | ARA_LEAF | p-Super | GC | 27 | 30 |
| 37503 | non-targ | B1259 | hentriacontane | ARA_LEAF | p-Super | GC | 27 | 72 |
| 155890 | plastidic | B1281 | hentriacontane | ARA_LEAF | p-Super | GC | 41 | 48 |
| 76890 | non-targ | B1343 | hentriacontane | ARA_LEAF | p-Super | GC | 24 | 52 |
| 38289 | plastidic | B1445 | hentriacontane | ARA_LEAF | p-Super | GC | 27 | 47 |
| 38300 | non-targ | B1597 | hentriacontane | ARA_LEAF | p-Super | GC | 38 | 96 |
| 38345 | plastidic | B1627 | hentriacontane | ARA_LEAF | p-Super | GC | 29 | 40 |
| 156392 | plastidic | B1649 | hentriacontane | ARA_LEAF | p-Super | GC | 28 | 48 |
| 78753 | non-targ | B1670 | hentriacontane | ARA_LEAF | p-Super | GC | 30 | 48 |
| 156542 | plastidic | B1704 | hentriacontane | ARA_LEAF | p-Super | GC | 25 | 83 |
| 79181 | non-targ | B1930 | hentriacontane | ARA_LEAF | p-Super | GC | 30 | 52 |
| 79217 | non-targ | B2027 | hentriacontane | ARA_LEAF | p-Super | GC | 26 | 43 |
| 104277 | non-targ | B2053 | hentriacontane | ARA_LEAF | p-Super | GC | 24 | 41 |
| 156810 | plastidic | B2207 | hentriacontane | ARA_LEAF | p-Super | GC | 32 | 43 |
| 146238 | non-targ | B2389 | hentriacontane | ARA_LEAF | p-Super | GC | 26 | 47 |
| 39237 | non-targ | B2399 | hentriacontane | ARA_LEAF | p-Super | GC | 97 | 168 |
| 39300 | non-targ | B2414 | hentriacontane | ARA_LEAF | p-Super | GC | 31 | 77 |
| 115628 | non-targ | B2439 | hentriacontane | ARA_LEAF | p-Super | GC | 28 | 49 |
| 156910 | plastidic | B2554 | hentriacontane | ARA_LEAF | p-Super | GC | 28 | 97 |
| 146288 | non-targ | B2653 | hentriacontane | ARA_LEAF | p-Super | GC | 28 | 43 |
| 81451 | non-targ | B2762 | hentriacontane | ARA_LEAF | p-Super | GC | 31 | 39 |
| 81576 | non-targ | B2818 | hentriacontane | ARA_LEAF | p-Super | GC | 24 | 125 |
| 157128 | non-targ | B2822 | hentriacontane | ARA_LEAF | p-Super | GC | 27 | 47 |
| 41499 | non-targ | B2957 | hentriacontane | ARA_LEAF | p-Super | GC | 38 | 65 |
| 157321 | plastidic | B2965 | hentriacontane | ARA_LEAF | p-Super | GC | 37 | 114 |
| 157450 | plastidic | B3385 | hentriacontane | ARA_LEAF | p-Super | GC | 35 | 97 |
| 125264 | plastidic | B3568 | hentriacontane | ARA_LEAF | p-Super | GC | 27 | 47 |
| 46850 | non-targ | GM02LC44512 | hentriacontane | ARA_LEAF | p-PcUBI | GC | 31 | 89 |
| 59370 | plastidic | Slr2124 | hentriacontane | ARA_LEAF | p-PcUBI | GC | 25 | 42 |
| 60301 | non-targ | TTC0035 | hentriacontane | ARA_LEAF | p-PcUBI | GC | 52 | 117 |
| 61553 | non-targ | TTC0917 | hentriacontane | ARA_LEAF | p-PcUBI | GC | 25 | 93 |
| 88879 | plastidic | Ybr249c | hentriacontane | ARA_LEAF | p-Super | GC | 31 | 88 |
| 64144 | non-targ | Yfl019c | hentriacontane | ARA_LEAF | Big35S | GC | 40 | 1100 |
| 120309 | non-targ | Yfl053w | hentriacontane | ARA_LEAF | Big35S | GC | 25 | 56 |
| 157717 | plastidic | YGRO55W | hentriacontane | ARA_LEAF | p-Super | GC | 26 | 41 |
| 157832 | plastidic | YHL039W | hentriacontane | ARA_LEAF | p-Super | GC | 25 | 53 |
| 157858 | non-targ | Yhr022c-a | hentriacontane | ARA_LEAF | Big35S | GC | 25 | 111 |
| 97631 | non-targ | Yjl127w-a | hentriacontane | ARA_LEAF | Big35S | GC | 36 | 78 |
| 157862 | plastidic | Ylr157c | hentriacontane | ARA_LEAF | p-Super | GC | 26 | 45 |
| 129546 | plastidic | Ynl321w | hentriacontane | ARA_LEAF | p-Super | GC | 25 | 57 |
| 113647 | plastidic | Ypr170c | hentriacontane | ARA_LEAF | p-Super | GC | 32 | 72 |
| 158033 | non-targ | YPR172W | hentriacontane | ARA_LEAF | Big35S | GC | 26 | 72 |
| 125348 | non-targ | B3644 | hentriacontane | ARA_LEAF | p-Super | GC | 8 | 450 |

[103144] Column 1 shows the SEQ ID NO, Column 2 shows the expression type (targeted or non-targeted), Column 3 shows the “gene name” (locus), Column 4 shows the metabolite analyzed, Column 5 indicates the A. thaliana source tissue analyzed, Column 6 indicates the used promoter for expression, Column 7 indicates the analytical method. Columns 8 and 9 show the minimum and the maximum increase of the analyzed metabolite (in percent) in comparison to the wild type (ratio_by_WT, given as percent increase).

[103145] The term “non-targ” in Table d, Column 2 which shows the expression type means “non-targeted”.

[103146] When the analyses were repeated independently, all results proved to be significant.

[103147] [0505.1.7.20] to [0515.1.7.20] for the disclosure of these paragraphs see [0505.1.7.7] to [0515.1.7.7] above

Lengthy table referenced here
US20140259212A1-20140911-T00001
Please refer to the end of the specification for access instructions.

Lengthy table referenced here
US20140259212A1-20140911-T00002
Please refer to the end of the specification for access instructions.

Lengthy table referenced here
US20140259212A1-20140911-T00003
Please refer to the end of the specification for access instructions.

Lengthy table referenced here
US20140259212A1-20140911-T00004
Please refer to the end of the specification for access instructions.

Lengthy table referenced here
US20140259212A1-20140911-T00005
Please refer to the end of the specification for access instructions.

Lengthy table referenced here
US20140259212A1-20140911-T00006
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20140259212A1). An electronic copy of the table will also be available from the USPTO upon request and payment of the fee set forth in 37 CFR 1.19(b)(3).

1-23. (canceled)

24. A process for the production of a fine chemical selected from the group consisting of the metabolites indicated in column 7 of Tables I, II and IV, comprising:
(a) increasing or generating one or more activities selected from the group consisting of activities as disclosed in paragraphs [0482.3.n.n], item 2, in a non-human organism or a part thereof, preferably a microorganism, a plant cell, a plant or a part thereof, as compared to a corresponding non-transformed wild type non-human organism or a part thereof; whereby n is a integer number from 1 to 20 and is found in the first column of Tables I, II and IV named “application number” and selected according to the respective metabolite indicated in column 7 of Tables I, II and IV; and
(b) growing the non-human organism or a part thereof under conditions which permit the production of the metabolite indicated in column 7 of Tables I, II and IV or a composition comprising the metabolite indicated in column 7 of Tables I, II and IV in said non-human organism or in the culture medium surrounding said non-human organism.

25. A process for the production of a fine chemical selected from the group consisting of the metabolites indicated in column 7 of Tables I, II and IV, which comprises:
(A)
(i) increasing or generating of the expression of;
(ii) increasing or generating the expression of an expression product of and/or
(iii) increasing or generating one or more activities of an expression product encoded by;
at least one respective nucleic acid molecule comprising a nucleic acid molecule selected from the group consisting of:
(a) a nucleic acid molecule encoding the polypeptide shown in column 5 or 8 of Table II, preferably Table II B, or a homolog or a fragment thereof;
(b) a nucleic acid molecule shown in column 5 or 8 of Table I, preferably Table I B, or a homolog or a fragment thereof (preferably the coding region thereof);
(c) a nucleic acid molecule, which, as a result of the degeneracy of the genetic code, can be derived from a polypeptide sequence depicted in column 5 or 8 of Table II, preferably Table II B;
(d) a nucleic acid molecule having at least 30%, 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, or the coding region thereof;
(e) a nucleic acid molecule encoding a polypeptide having at least 30%, 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV;
(i) a nucleic acid molecule encoding a polypeptide having the activity represented by a respective protein as depicted in column 5 of Table II;
(j) a nucleic acid molecule which comprises a polynucleotide, which is obtained by amplifying a cDNA library or a genomic library using the primers as depicted in column 8 of Table III; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j); or a nucleic acid molecule comprising a sequence which is complementary thereto;
in a non-human organism or a part thereof, preferably a microorganism, a plant cell, a plant or a part thereof, as compared to a corresponding non-transformed wild type non-human organism or a part thereof; and
(B) growing the non-human organism or a part thereof under conditions which permit the production of the respective metabolite indicated in column 7 of Tables I, II and IV or a composition comprising the respective metabolite indicated in column 7 of Tables I, II and IV in said non-human organism or in the culture medium surrounding said non-human organism.

26. A process of claim 24, comprising of recovering the respective metabolite indicated in column 7 of Tables I, II and IV in its free or bound form.

27. A process for the production of a fine chemical selected from the group consisting of the metabolites indicated in column 7 of Tables I, II and IV, comprising:
(a) selecting a non-human organism or a part thereof expressing a polypeptide encoded by the nucleic acid molecule characterized in claim 25;
(b) mutagenizing the selected non-human organism or the part thereof;
(c) comparing the activity or the expression level of said polypeptide in the mutagenized non-human organism or the part thereof with the activity or the expression of said polypeptide of the selected non-human organisms or the part thereof;
(d) selecting the mutated non-human organisms or parts thereof, which comprise an increased activity or expression level of said polypeptide compared to the selected non-human organism or the part thereof;
(e) optionally, growing and cultivating the non-human organisms or the parts thereof; and
(f) recovering, and optionally isolating, the free or bound the respective metabolite indicated in column 7 of Tables I, II and IV produced by the selected mutated non-human organisms or parts thereof.

28. The process of claim 27, wherein the activity of said protein or the expression of said nucleic acid molecule is increased or generated transiently or stably.

29. An isolated nucleic acid molecule comprising a nucleic acid molecule selected from the group consisting of:
(a) a nucleic acid molecule encoding a polypeptide shown in Table II, column 5 or 8, preferably shown in Table II B, column 8, or a homolog or a fragment thereof;
(b) a nucleic acid molecule shown in Table I, column 5 or 8, preferably shown in Table I B, column 8, preferably the coding region thereof, or a homolog or a fragment thereof;
(c) a nucleic acid molecule, which, as a result of the degeneracy of the genetic code, can be derived from a polypeptide sequence depicted in column 5 or 8 of Table II, preferably in column 8 of Table II B;
(d) a nucleic acid molecule having at least 30%, 40%, 50%, 60%, 70%, 75%, 80%, 85%, 90%, 95%, 96%, 97%, 98%, 99% or 99.5% identity with the nucleic acid molecule sequence of a polynucleotide comprising the nucleic acid molecule shown in column 5 or 8 of Table I, preferably shown in column 8 of Table I B, preferably the coding region thereof;
(e) a nucleic acid molecule encoding a polypeptide having at least 30%, 40%, 50%, 60%, 70%, 75%, 80%, 85%, 90%, 95%, 96%, 97%, 98%, 99% or 99.5% identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV;
(i) a nucleic acid molecule encoding a polypeptide having the activity represented by a respective protein as depicted in column 5 of Table II;
(j) a nucleic acid molecule comprising a polynucleotide which is obtained by amplifying a cDNA library or a genomic library using the primers in Table III, column 8;
and
(k) a nucleic acid molecule which is obtainable by screening a suitable nucleic acid library, especially a cDNA library and/or a genomic library, under stringent hybridization conditions with a probe comprising one of the sequences of the nucleic acid molecule of (a), (b), (c), (d), (e), (f), (g), (h), (i) or (j) or with a fragment of at least 15 nt, 20 nt, 3Ont, 50 nt, 100 nt, 200 nt,500 nt, 1000 nt, 1500 nt, 2000 nt or 3000 nt of the nucleic acid molecule characterized in (a), (b), (c), (d), (e), (f), (g), (h), (i) or (j);
or a nucleic acid molecule comprising a sequence which is complementary thereto.

30. A nucleic acid construct which confers expression of the nucleic acid molecule of claim 29, comprising one or more regulatory elements.

31. An expression cassette comprising:
(a) a promoter, preferably selected from the group consisting of Big35S, PCUbi, Super and USP; and
(b) the nucleic acid molecule of claim 29.

32. A vector comprising the nucleic acid molecule of claim 29, a nucleic acid construct conferring expression of said nucleic acid molecule and comprising one or more regulatory elements, or an expression cassette comprising a promoter and said nucleic acid molecule.

33. A host cell or a plant organelle transformed with the nucleic acid molecule of claim 29, a nucleic acid construct conferring expression of said nucleic acid molecule and comprising one or more regulatory elements, an expression cassette comprising a promoter and said nucleic acid molecule, or a vector comprising said nucleic acid molecule, said nucleic acid construct, or said expression cassette.

34. The host cell of claim 33, wherein the host cell is a plant cell or a microorganism.

35. A process for producing a polypeptide, comprising expressing said polypeptide in the host cell of claim 33.

36. A polypeptide encoded by the nucleic acid molecule of claim 29, or produced by a process comprising expressing said polypeptide in a host cell transformed with said nucleic acid molecule.

37. An antibody, which binds specifically to the polypeptide of claim 36.

38. A plant tissue, propagation material, harvested material, or a plant or part thereof, comprising the host cell of claim 33.

39. A process for the identification of a compound conferring an increase in a respective metabolite indicated in column 7 of Tables I, II and IV production in a non-human organism, comprising:
(a) culturing a plant cell or tissue or microorganism or maintaining a plant expressing the polypeptide encoded by the nucleic acid molecule of claim 29, conferring an increase in the amount of the respective metabolite indicated in column 7 of Tables I, II and IV in a non-human organism or a part thereof and a readout system capable of interacting with the polypeptide under suitable conditions which permit the interaction of the polypeptide with said readout system in the presence of a compound or a sample comprising a plurality of compounds and capable of providing a detectable signal in response to the binding of a compound to said polypeptide under conditions which permit the expression of said readout system and of the polypeptide encoded by said nucleic acid molecule conferring an increase in the amount of the respective metabolite indicated in column 7 of Tables I, II and IV a non-human organism or a part thereof;
(b) identifying if the compound is an effective agonist by detecting the presence or absence or increase of a signal produced by said readout system.

40. A method for the production of an agricultural composition, comprising obtaining a compound according to the method of claim 39, and formulating said compound in a form acceptable for an application in agriculture.

41. A composition comprising the nucleic acid molecule of claim 29, a polypeptide encoded by said nucleic acid molecule, a nucleic acid construct conferring expression of said nucleic acid molecule and comprising one or more regulatory elements, an expression cassette comprising a promoter and said nucleic acid molecule, a vector comprising said nucleic acid molecule, said nucleic acid construct, or said expression cassette, a compound identified by said polypeptide, or an antibody which binds specifically to said polypeptide, and optionally an agricultural acceptable carrier.

42. A method for the identification of a nucleic acid molecule conferring an increase in the respective metabolite(s) indicated in column 7 of Tables I, II and IV after expression, comprising utilizing the nucleic acid molecule of claim 29.

43. A cosmetic, pharmaceutical, food or feed composition comprising the nucleic acid molecule of claim 29, a polypeptide encoded by said nucleic acid molecule, a nucleic acid construct conferring expression of said nucleic acid molecule and comprising one or more regulatory elements, an expression cassette comprising a promoter and said nucleic acid molecule, a vector comprising said nucleic acid molecule, said nucleic acid construct, or said expression cassette, an antibody which binds specifically to said polypeptide, a host cell transformed with said nucleic acid molecule, said nucleic acid construct, said expression cassette, or said vector, a plant or part thereof comprising said host cell, or a plant tissue, harvested material or propagation material of said plant.

44. A method for the production of a plant resistant to a herbicide inhibiting the production of the respective metabolite(s) indicated in column 7 of Tables I, II and IV, comprising utilizing the nucleic acid molecule of claim 29, a polypeptide encoded by said nucleic acid molecule, a nucleic acid construct conferring expression of said nucleic acid molecule and comprising one or more regulatory elements, an expression cassette comprising a promoter and said nucleic acid molecule, a vector comprising said nucleic acid molecule, said nucleic acid construct, or said expression cassette, a host cell transformed with said nucleic acid molecule, said nucleic acid construct, said expression cassette, or said vector, or a plant or plant tissue comprising said host cell.

45. A method for producing a plant with increased yield as compared to a corresponding non-transformed wild type plant, comprising:
(i) increasing or generating expression of;
(ii) increasing or generating expression of an expression product of; and/or
(iii) increasing or generating one or more activities of an expression product encoded by; at least one respective nucleic acid molecule comprising a nucleic acid molecule selected from the group consisting of:
(a) a nucleic acid molecule encoding the polypeptide shown in column 5 or 8 of Table II, preferably Table II B, or a homolog or a fragment thereof, in case in column 7 the metabolite from paragraph [0482.3.n.n], item 22; is indicated; whereby n is a integer number from 1 to 20 and is found in the first column of Tables I, II and IV named “application number” and selected according to respective the metabolites indicated in paragraph [0482.3.n.n], item 22;
(b) a nucleic acid molecule shown in column 5 or 8 of Table I, preferably Table I B, or a homolog or a fragment thereof (preferably the coding region thereof), in case in column 7 the metabolite from paragraph [0482.3.n.n], item 22; is indicated; whereby n is a integer number from 1 to 20 and is found in the first column of Tables I, II and IV named “application number” and selected according to respective the metabolites indicated in paragraph [0482.3.n.n], item 22;
(c) a nucleic acid molecule, which, as a result of the degeneracy of the genetic code, can be derived from a polypeptide sequence depicted in column 5 or 8 of Table II, preferably Table II B, in case in column 7 the metabolite from paragraph [0482.3.n.n], item 22; is indicated; whereby n is a integer number from 1 to 20 and is found in the first column of Tables I, II and IV named “application number” and selected according to respective the metabolites indicated in paragraph [0482.3.n.n], item 22;
(d) a nucleic acid molecule having at least 30%, 50%, 60%, 70%, 80%, 85%, 90%, 95%, 97%, 98%, 99% or 99.5% identity with the nucleic acid molecule sequence of a polynucleotide comprising the nucleic acid molecule shown in column 5 or 8 of Table I, preferably Table I B, or the coding region thereof, in case in column 7 the metabolite from paragraph [0482.3.n.n], item 22; is indicated; whereby n is a integer number from 1 to 20 and is found in the first column of Tables I, II and IV named “application number” and selected according to respective the metabolites indicated in paragraph [0482.3.n.n], item 22;
(e) a nucleic acid molecule encoding a polypeptide having at least 30%, 50%, 60%, 70%, 80%, 85%, 90%, 95%, 97%, 98%, 99% or 99.5% identity with the amino acid sequence of the polypeptide encoded by the nucleic acid molecule of (a), (b), (c) or (d);
(f) a nucleic acid molecule which hybridizes with a nucleic acid molecule of (a), (b), (c), (d) or (e) under stringent hybridization conditions;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f);
(h) a nucleic acid molecule encoding a polypeptide comprising the consensus sequence or one or more polypeptide motifs as shown in column 8 of Table IV, in case in column 7 the metabolite from paragraph [0482.3.n.n], item 22; is indicated; whereby n is a integer number from 1 to 20 and is found in the first column of Tables I, II and IV named “application number” and selected according to respective the metabolites indicated in paragraph [0482.3.n.n], item 22;
(i) a nucleic acid molecule encoding a polypeptide having the activity represented by a respective protein as depicted in column 5 of Table II, in case in column 7 the metabolite from paragraph [0482.3.n.n], item 22; is indicated; whereby n is a integer number from 1 to 20 and is found in the first column of Tables I, II and IV named
(j) a nucleic acid molecule which comprises a polynucleotide, which is obtained by amplifying a cDNA library or a genomic library using the primers as depicted in column 8 of Table III, in case in column 7 the metabolite from paragraph [0482.3.n.n], item 22; is indicated; whereby n is a integer number from 1 to 20 and is found in the first column of Tables I, II and IV named “application number” and selected according to respective the metabolites indicated in paragraph [0482.3.n.n], item 22 is indicated; and
(k) a nucleic acid molecule which is obtainable by screening a suitable nucleic acid library, especially a cDNA library and/or a genomic library, under stringent hybridization conditions with a probe comprising a complementary sequence of a nucleic acid molecule of (a), (b), (c), (d), (e), (f), (g), (h), (i) or (j) or with a fragment thereof, having at least 15 nt, 20 nt, 30 nt, 50 nt, 100 nt, 200 nt, 500 nt, 1000 nt, 1500 nt, 2000 nt or 3000 nt of a nucleic acid molecule complementary to a nucleic acid molecule sequence characterized in (a), (b), (c), (d), (e), (f), (g), (h), (i) or (j);
or a nucleic acid molecule comprising a sequence which is complementary thereto; in a plant cell, plant or a part thereof.

46. A method for producing a plant with increased yield according to claim 45, further comprising regenerating a plant having increased yield from the transformed plant cell, plant cell compartment, plant cell nucleus, plant cell or plant tissue.
